(19) United States
(12) Patent Application Publication
(10) Pub. No.: US 2023/0107430 A1
(43) Pub. Date: Apr. 6, 2023

(54) CONDENSED CYCLIC COMPOUND, ORGANIC LIGHT-EMITTING DEVICE INCLUDING THE SAME, AND ELECTRONIC APPARATUS INCLUDING THE ORGANIC LIGHT-EMITTING DEVICE

(71) Applicant: Samsung Electronics Co., Ltd., Suwon-si (KR)

(72) Inventors: Yongsik JUNG, Seoul (KR); Sangho PARK, Anyang-si (KR); Eunsuk KWON, Suwon-si (KR); Sangmo KIM, Hwaseong-si (KR); Hwang Suk KIM, Suwon-si (KR); Jiwhan KIM, Seoul (KR); Sungho NAM, Daegu (KR); Sooghang IHN, Hanam-si (KR); Soonok JEON, Suwon-si (KR); Kyeongsik JU, Suwon-si (KR)

(21) Appl. No.: 17/725,942

(22) Filed: Apr. 21, 2022

(30) Foreign Application Priority Data
Apr. 23, 2021 (KR) 10-2021-0053283
Jan. 27, 2022 (KR) 10-2022-0012592

(51) Int. Cl. C07F 5/02; H01L 51/00; C07F 9/50; C07F 9/53; C07D 487/14; C07D 471/14; C07D 519/00

(52) U.S. Cl. CPC C07F 5/027; H01L 51/0061; C07F 9/5018; C07F 9/532; C07D 487/14; C07D 471/14; H01L 51/008; H01L 51/0072; C07D 519/00; H01L 51/0067; H01L 51/5028

(57) ABSTRACT

Provided are a condensed cyclic compound represented by Formula 1, an organic light-emitting device including the same, and an electronic apparatus including the organic light-emitting device.

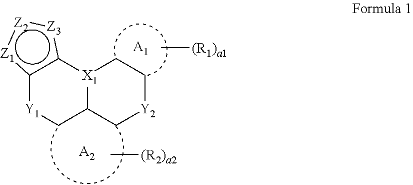

The detailed description of Formula 1 is the same as described in the present specification.

### CROSS-REFERENCE TO RELATED APPLICATION

This application claims priority under 35 U.S.C. § 119 to Korean Patent Application Nos. 10-2021-0053283, filed on Apr. 23, 2021, and 10-2022-0012592, filed on Jan. 27, 2022, in the Korean Intellectual Property Office, the contents of which are incorporated by reference herein in their entirety.

### BACKGROUND

### 1. Field

The present disclosure relates to condensed cyclic compounds, organic light-emitting devices including the same, and electronic apparatuses including the organic light-emitting devices.

### 2. Description of the Related Art

Organic light-emitting devices are self-emissive devices, which have improved characteristics in terms of viewing angles, response time, brightness, driving voltage, and response speed, and produce full-color images.

In an example, an organic light-emitting device includes an anode, a cathode, and an organic layer located between the anode and the cathode, wherein the organic layer includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transition from an excited state to a ground state, thus generating light.

### SUMMARY

Provided are novel condensed cyclic compounds, organic light-emitting devices including the same, and electronic apparatuses including the organic light-emitting devices.

Additional aspects will be set forth in part in the description which follows and, in part, will be apparent from the description, or may be learned by practice of the presented embodiments of the disclosure.

According to one aspect, provided is a condensed cyclic compound represented by Formula 1:

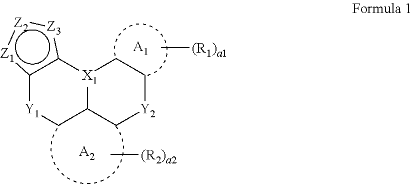

wherein, in Formula 1,

X1 is B, N, P, or P(═O),

Z₁ to Z₃ are each independently be O, S, N, N(R₃), or C(R₄), wherein at least two of Z₁ to Z₃ are each independently O, S, N, or N(R₃),

a ring including Z₁ to Z₃ is a 5-membered heteroaromatic ring,

Y₁ and Y₂ are each independently N[(L₁₁)_{b11}-(R₅)ₐ₅], O, S, Se, B(R₆), or C(═O), wherein Y₁ and Y₂ may be identical to or different from each other,

A₁ and A₂ are each independently a C₅-C₃₀ carbocyclic group or a C₁-C₃₀ heterocyclic group,

L₁₁ is a substituted or unsubstituted C₅-C₃₀ carbocyclic group or a substituted or unsubstituted C₁-C₃₀ heterocyclic group,

b11 is 0, 1, or 2,

R₁ to R₆ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

a1, a2, and a5 are each independently an integer from 1 to 10,

a substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or any combination thereof;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or any combination thereof;

—N(Q₃₁)(Q₃₂), —Si(Q₃₃)(Q₃₄)(Q₃₅), —B(Q₃₆)(Q₃₇), or —P(═O)(Q₃₈)(Q₃₉); or

any combination thereof,

wherein Q₁ to Q₉, Q₁₁ to Q₁₉, Q₂₁ to Q₂₉, and Q₃₁ to Q₃₉ may each independently be: hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₁-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₁-C₆₀ heteroaryl group; a monovalent non-aromatic condensed polycyclic group; or a monovalent non-aromatic condensed heteropolycyclic group.

According to another aspect, provided is an organic light-emitting device including a first electrode, a second electrode, and an organic layer including an emission layer and located between the first electrode and the second electrode, wherein the organic layer includes at least one of the condensed cyclic compound.

Another aspect of the present disclosure provides an electronic apparatus including the organic light-emitting device.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a schematic cross-sectional view of an organic light-emitting device according to an exemplary embodiment; and

FIGS. 2A to 2E show diagrams schematically illustrating an energy transfer process according to an exemplary embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween In contrast, when an element is referred to as being “directly on” another element, there are no intervening elements present

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded Thus, the regions illustrated in the figures are schematic in nature and their shapes are not intended to illustrate the precise shape of a region and are not intended to limit the scope of the present claims.

An aspect of the present disclosure provides a condensed cyclic compound represented by Formula 1:

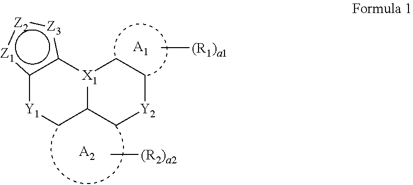

X₁ in Formula 1 may be B, N, P, or P(═O).

Z₁ to Z₃ in Formula 1 may each independently be O, S, N, N(R₃), or C(R₄), and at least two of Z₁ to Z₃ may be O, S, N, or N(R₃).

A ring including Z₁ to Z₃ may be a 5-membered heteroaromatic ring.

In an embodiment, the ring including Z₁ to Z₃ may be an imidazole group, an oxazole group, a thiazole group, a pyrazole group, an isoxazole group, an isothiazole group, a triazole group, an oxadiazole group, or a thiadiazole group.

In an embodiment, the ring including Z₁ to Z₃ may be one of the groups represented by Formulae A3-1 to A3-27:

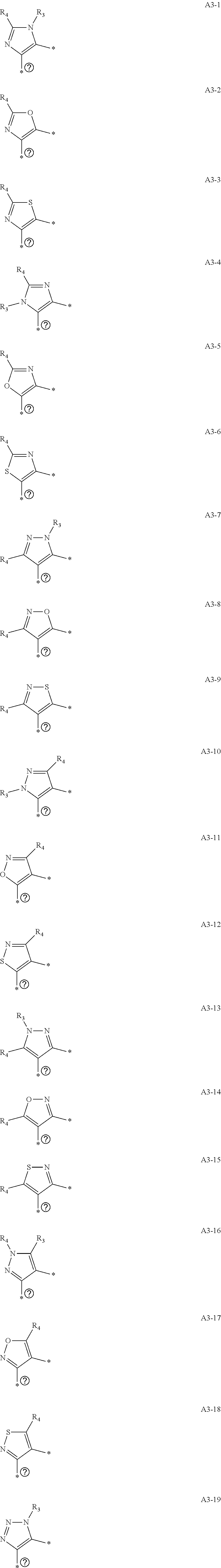

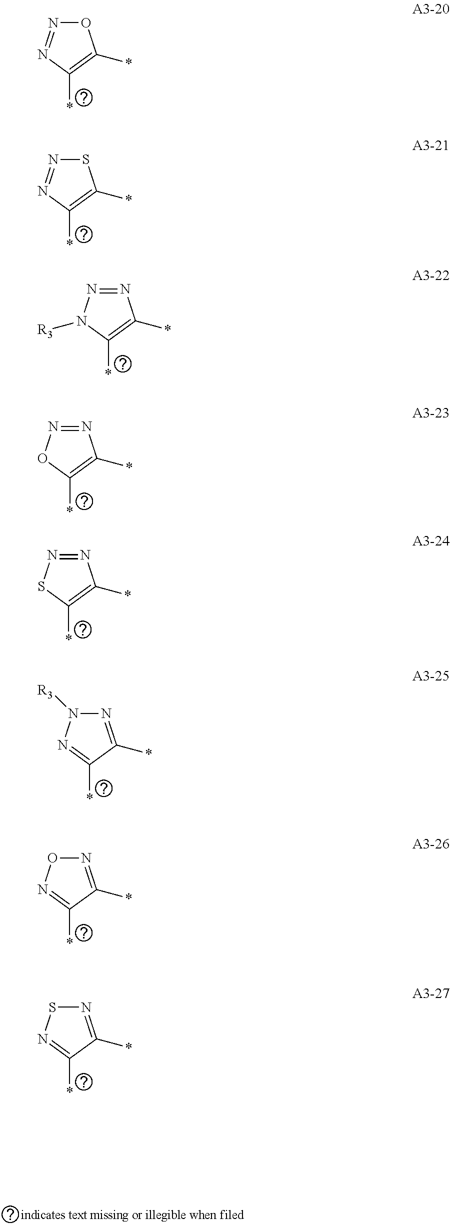

wherein, in Formulae A3-1 to A3-27,

R₃ and R₄ are each the same as described in the present specification,

* indicates a binding site to X₁,

*′ indicates a binding site to Y₁.

For example, R₃ in Formulae A3-1 to A3-17 may be a cyclopentyl, a cyclohexyl group, a cyclopentenyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, or a dibenzothiophenyl group, each unsubstituted or substituted with deuterium, —F, a cyano group, a C₁-C₂₀ alkyl group, a C₁-C₂₀ alkoxy group, a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof,

R₄ may be hydrogen, deuterium, —F, or a cyano group;

a C₁-C₂₀ alkyl group or a C₁-C₂₀ alkoxy group, each unsubstituted or substituted with deuterium, —F, a cyano group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof; or

a cyclopentyl group, a cyclohexyl group, a cyclopentyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, or a dibenzothiophenyl group, each unsubstituted or substituted with deuterium, —F, a cyano group, a C₁-C₂₀ alkyl group, a C₁-C₂₀ alkoxy group, a cyclopentyl group, a cyclohexyl group, a cyclopentyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof.

Y₁ and Y₂ in Formula 1 may each independently be N[(L₁₁)_{b11}-(R₅)ₐ₅], O, S, Se, B(R₆), or C(═O), and Y₁ and Y₂ may be identical to or different from each other.

In an embodiment, X₁ may be B, and Y₁ and Y₂ may each independently be N[(L₁₁)_{b11}-(R₅)ₐ₅], O, S, Se, or C(═O). In an embodiment, X₁ may be P or P(═O), and Y₁ and Y₂ may each independently be N[(L₁₁)_{b11}-(R₅)ₐ₅]. In an embodiment, X₁ may be N, and Y₁ and Y₂ may each independently be N[(L₁₁)_{b11}-(R₅)ₐ₅], B(R₆) or C(═O).

A₁ and A₂ in Formula 1 may each independently be a C₅-C₃₀ carbocyclic group or a C₁-C₃₀ heterocyclic group.

In an embodiment, A₁ and A₂ in Formula 1 may each independently be a benzene group, a naphthalene group, a 1,2,3,4-tetrahydronaphthalene group, a fluorene group, a carbazole group, a benzofuran group, a dibenzofuran group, a benzothiophene group, a dibenzothiophene group, a benzosilole group, a dibenzosilole group, a pyridine group, a pyrimidine group, a pyrazine group, a pyridazine group, a quinoline group, an isoquinoline group, a quinoxaline group, a quinazoline group, an imidazole group, an oxazole group, a thiazole group, a pyrazole group, an isoxazole group, an isothiazole group, a triazole group, an oxadiazole, or a thiadiazole group.

For example, A₁ in Formula 1 may be a benzene group, a pyridine group, a pyrimidine group, a pyrazine group, a pyridazine group, an imidazole group, an oxazole group, a thiazole group, a pyrazole group, an isoxazole group, an isothiazole group, a triazole group, an oxadiazole, or a thiadiazole group, and A₂ may be a benzene group.

L₁₁ in Formula 1 may be a substituted or unsubstituted C₅-C₃₀ carbocyclic group or a substituted or unsubstituted C₁-C₃₀ heterocyclic group.

In an embodiment, L₁₁ in Formula 1 may be a phenylene group, an indenylene group, a naphthylene group, an azulenylene group, a heptalenylene group, a acenaphthylene group, a fluorenylene group, a phenalenylene group, a phenanthrenylene group, an anthracenylene group, a fluoranthenylene group, a triphenylenylene group, a pyrenylene group, or a chrysenylene group, each unsubstituted or substituted with deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, C₁-C₂₀ alkyl group, a C₂-C₂₀ alkenyl group, a C₂-C₂₀ alkynyl group, a C₁-C₂₀ alkoxy group, a phenyl group, an indenyl group, a naphthyl group, an azulenyl group, a heptalenyl group, an acenaphthyl group, a fluorenyl group, a phenalenyl group, a phenanthrenyl group, an anthracenyl group, a fluoranthenyl group, a triphenylenyl group, a pyrenyl group, a chrysenylenyl group, or any combination thereof.

For example, L₁₁ may be of Formulae 3-1 or 3-32, and embodiments of the present disclosure are not limited thereto:

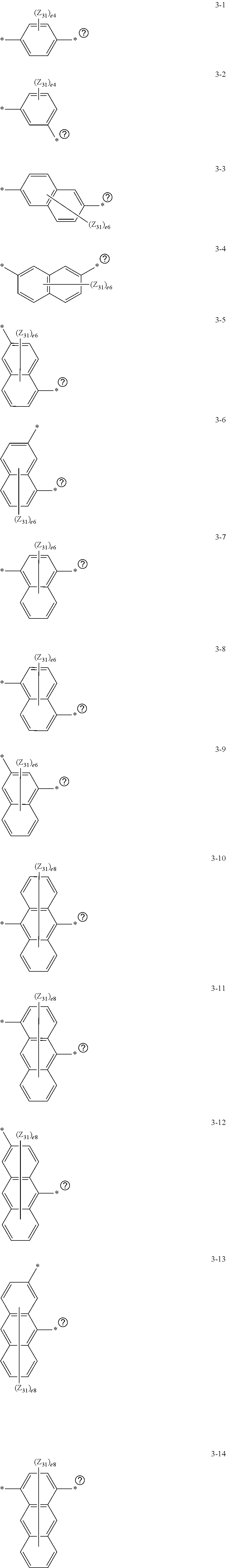

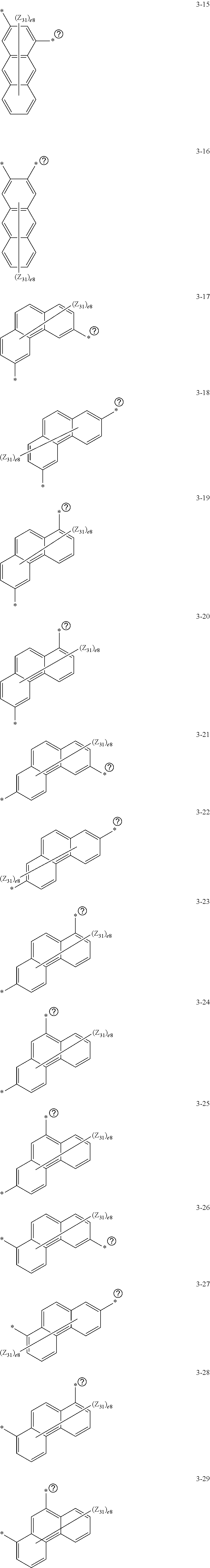

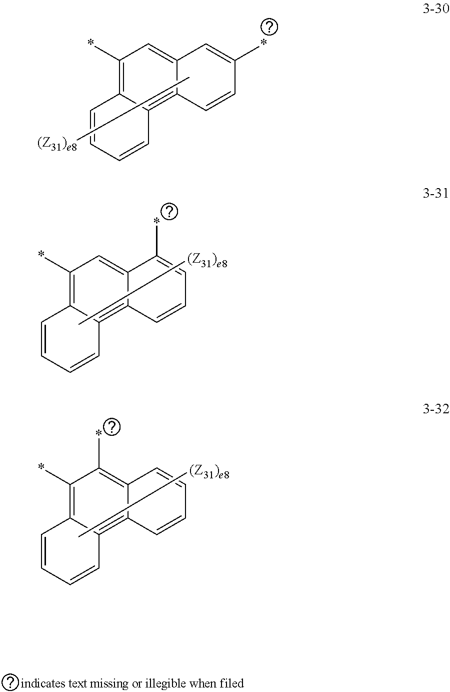

wherein, in Formulae 3-1 to 3-32,

Z₃₁ may be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₆-C₆₀ aryl alkyl group, or a C₁-C₆₀ heteroaryl group,

e4 may be an integer from 1 to 4,

e6 may be an integer from 1 to 6,

e8 may be an integer from 1 to 8, and

* and *′ each indicate a binding site to an adjacent atom.

b11 in Formula 1 may be 0, 1, or 2.

In an embodiment, the condensed cyclic compound may be represented by Formula 1A or 1B:

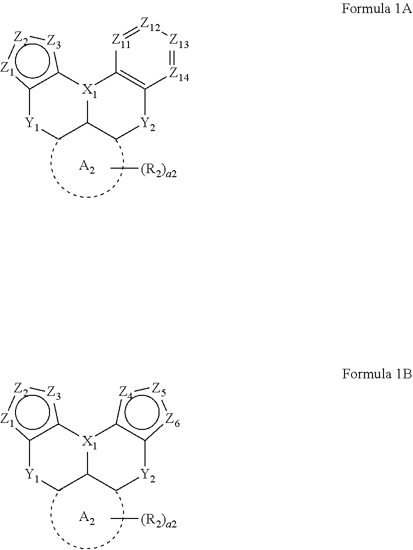

wherein, in Formulae 1A and 1B,

Z₁₁ may be C(R₁₁) or N, Z₁₂ may be C(R₁₂) or N, Z₁₃ may be C(R₁₃) or N, Z₁₄ may be C(R₁₄) or N,

Z₄ to Z₆ may each independently be O, S, N, N(R₁₁), or C(R₁₂), wherein at least two of Z₄ to Z₆ may each independently be O, S, N, or N(R₁₁),

a ring including Z₄ to Z₆ may be a 5-membered heteroaromatic ring,

R₁₁ to R₁₄ are each the same as described in connection with R₁, and

X₁, Z₁ to Z₃, Y₁, Y₂, A₂, R₂, and a2 are each the same as described in the present specification.

In an embodiment, the ring including Z₄ to Z₆ in Formula 1B may be one of the groups represented by Formulae A1-1 to A1-27:

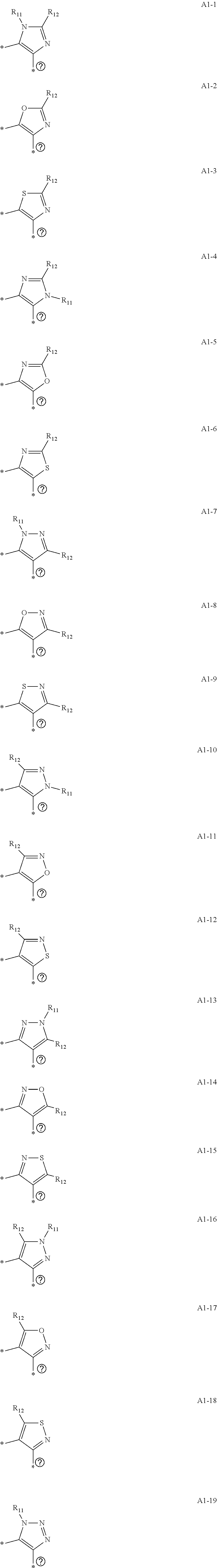

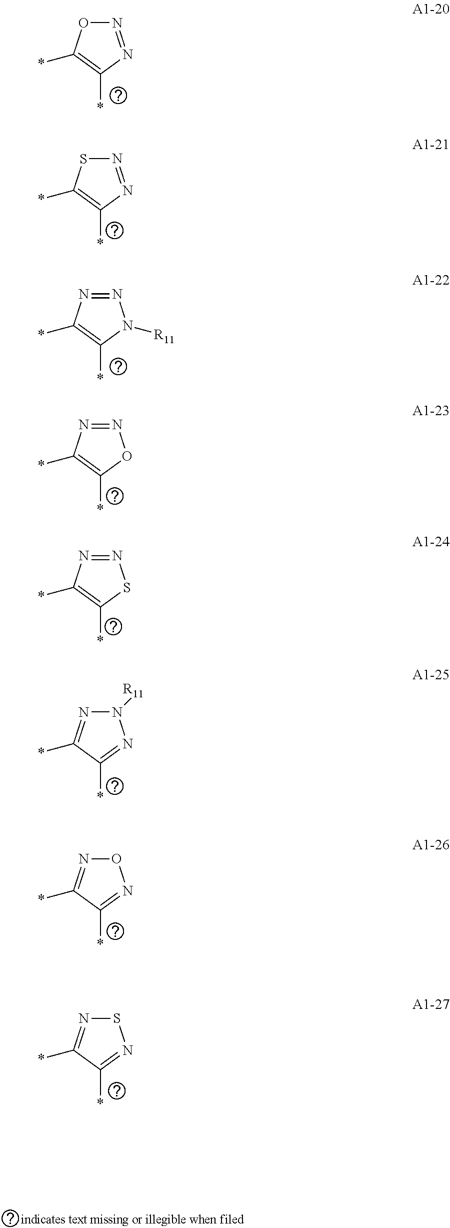

wherein, in Formulae A1-1 to A1-27,

R₁₁ and R₁₂ are the same as described in connection with R₁,

* indicates a binding site to X₁, and

*′ indicates a binding site to Y₂.

R₁ to R₆ in Formula 1 may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉).

In an embodiment, R₅ and R₆ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group.

In an embodiment, R₁ to R₆ may each independently be:

hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof;

a C₁-C₂₀ alkyl group or a C₁-C₂₀ alkoxy group, each unsubstituted or substituted with deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, —N(Q₃₄)(Q₃₅), or any combination thereof.

a cyclopentyl group, a cyclohexyl group, a cyclopentenyl group, a cyclohexenyl group, a cycloheptenyl group, a phenyl group, a naphthyl group, a pentalenyl group, an indenyl group, an azulenyl group, a heptalenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a pyrrolyl group, an imidazolyl group, a pyrazolyl group, a pyridinyl group, a pyrazinyl group, a pyrimidinyl group, a pyridazinyl group, a triazinyl group, an indolyl group, an isoindolyl group, a benzimidazolyl group, an indazolyl group, a carbazolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a furanyl group, a thiophenyl group, an oxazolyl group, an isoxazolyl group, a thiazolyl group, an isothiazolyl group, a triazolyl group, a tetrazolyl group, an oxadiazolyl group, a thiadiazolyl group, a silolyl group, a benzofuranyl group, a benzothiophenyl group, a benzoxazolyl group, a benzothiazolyl group, a benzosilolyl group, a dibenzofuranyl group, a dibenzothiophenyl group, or a dibenzosilolyl group, each unsubstituted or substituted with deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cyclopentenyl group, a cyclohexenyl group, a cycloheptenyl group, a phenyl group, a naphthyl group, a pentalenyl group, an indenyl group, an azulenyl group, a heptalenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a pyrrolyl group, an imidazolyl group, a pyrazolyl group, a pyridinyl group, a pyrazinyl group, a pyrimidinyl group, a pyridazinyl group, a triazinyl group, an indolyl group, an isoindolyl group, a benzimidazolyl group, an indazolyl group, a carbazolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a furanyl group, a thiophenyl group, an oxazolyl group, an isoxazolyl group, a thiazolyl group, an isothiazolyl group, a triazolyl group, a tetrazolyl group, an oxadiazolyl group, a thiadiazolyl group, a silolyl group, a benzofuranyl group, a benzothiophenyl group, a benzoxazolyl group, a benzothiazolyl group, a benzosilolyl group, a dibenzofuranyl group, a dibenzothiophenyl group, a dibenzosilolyl group, —N(Q₃₄)(Q₃₅), or any combination thereof; or

—N(Q₁)(Q₂),

wherein Q₁, Q₂, Q₃₄, and Q₃₅ may each independently be: a C₁-C₁₀ alkyl group; a C₁-C₁₀ alkoxy group; a phenyl group, a biphenyl group, a terphenyl group, or a naphthyl group, each unsubstituted or substituted with deuterium, a C₁-C₁₀ alkyl group, a phenyl group, or any combination thereof.

In an embodiment, R₁ to R₆ may each independently be:

hydrogen, deuterium, —F, or a cyano group;

a C₁-C₂₀ alkyl group or a C₁-C₂₀ alkoxy group, each unsubstituted or substituted with deuterium, —F, a cyano group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof;

a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, or a dibenzothiophenyl group, each unsubstituted or substituted with deuterium, —F, a cyano group, a C₁-C₂₀ alkyl group, a C₁-C₂₀ alkoxy group, a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof; or

—N(Q₁)(Q₂),

wherein Q₁ and Q₂ may each independently be: a C₁-C₁₀ alkyl group; a C₁-C₁₀ alkoxy group; a phenyl group, a biphenyl group, a terphenyl group, or a naphthyl group, each unsubstituted or substituted with deuterium, a C₁-C₁₀ alkyl group, a phenyl group, or any combination thereof.

In an embodiment, R₁ to R₆ in Formula 1 may each independently be hydrogen, deuterium, —F, a cyano group, a group represented by one of Formulae 9-1 to 9-19, or a group represented by one of Formulae 10-1 to 10-215:

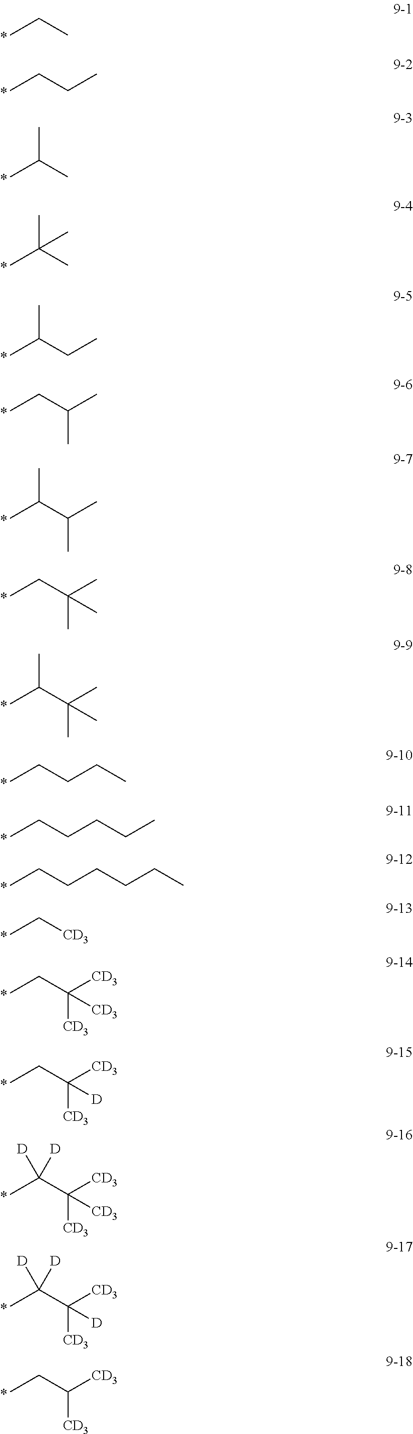

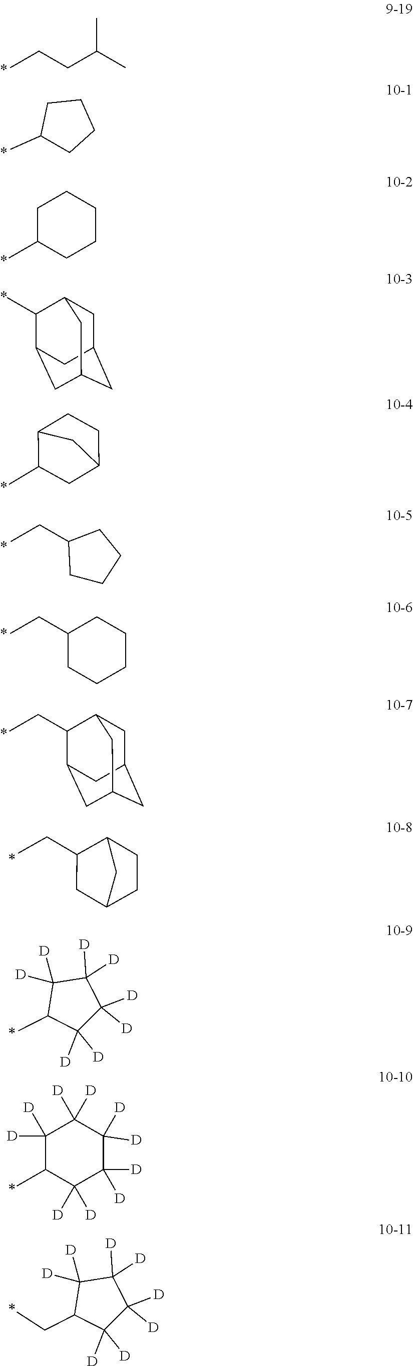

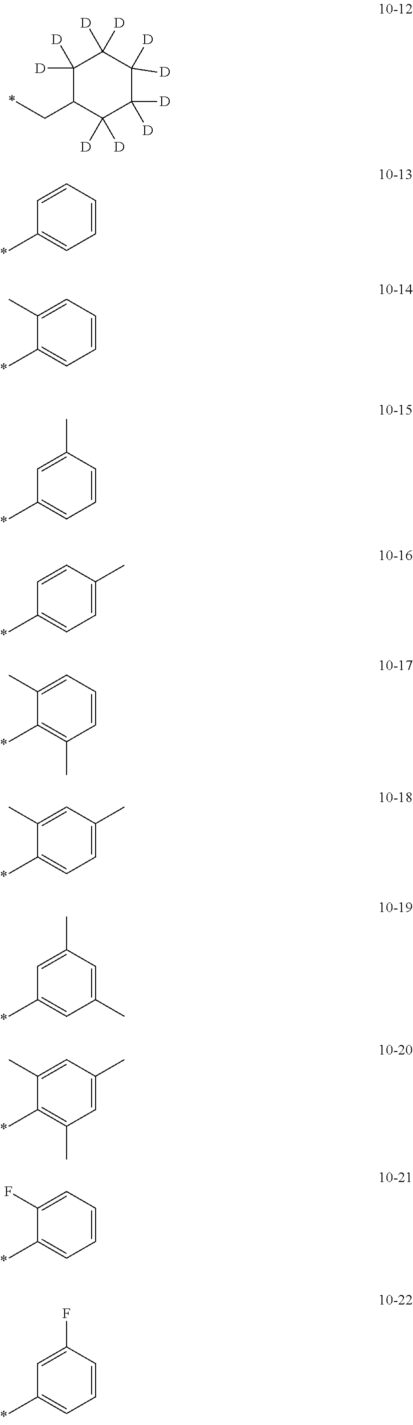

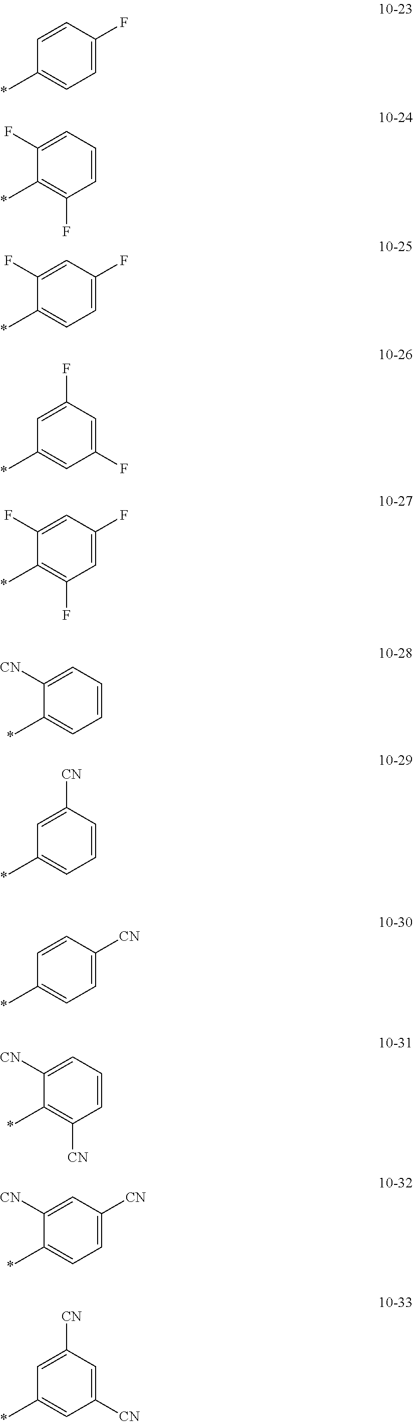

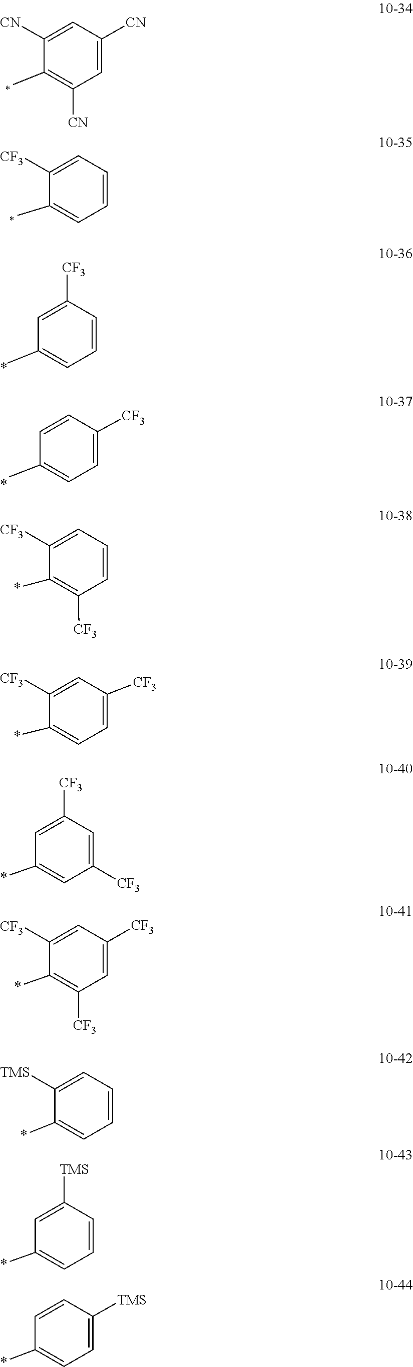

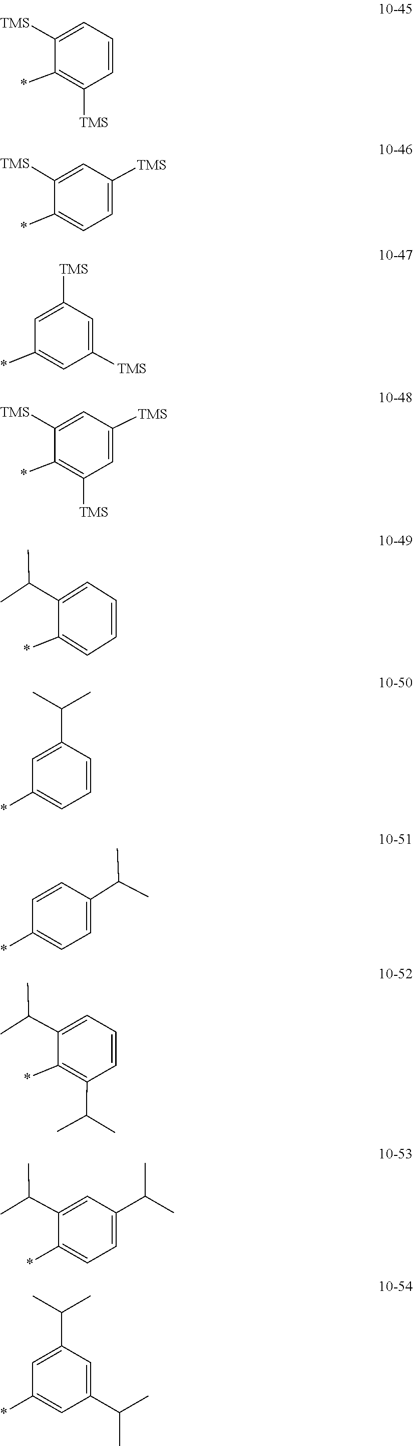

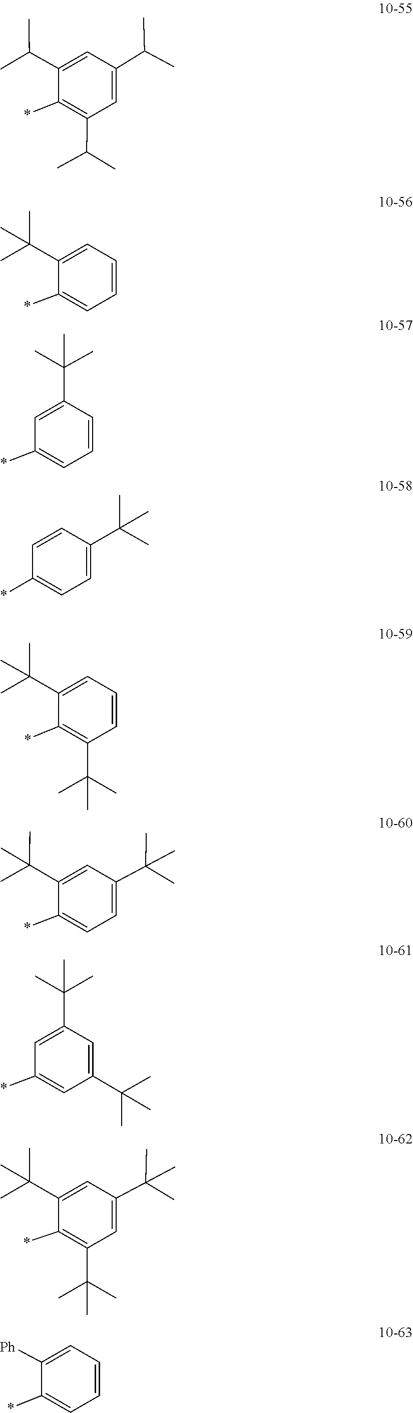

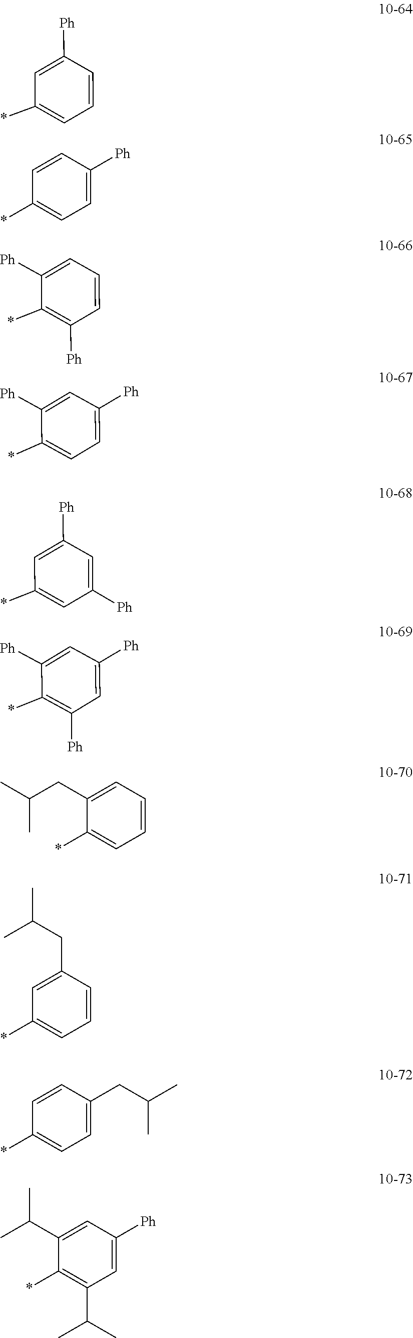

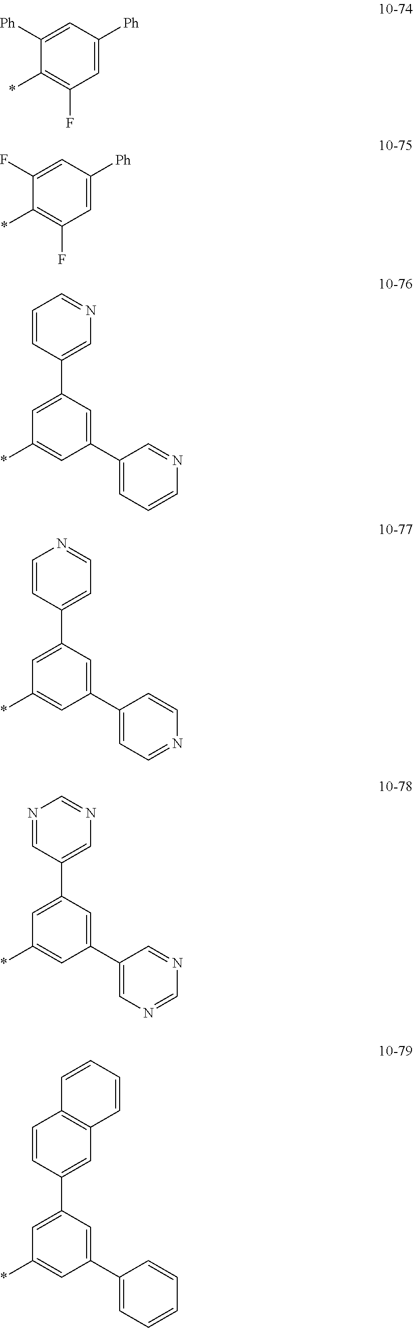

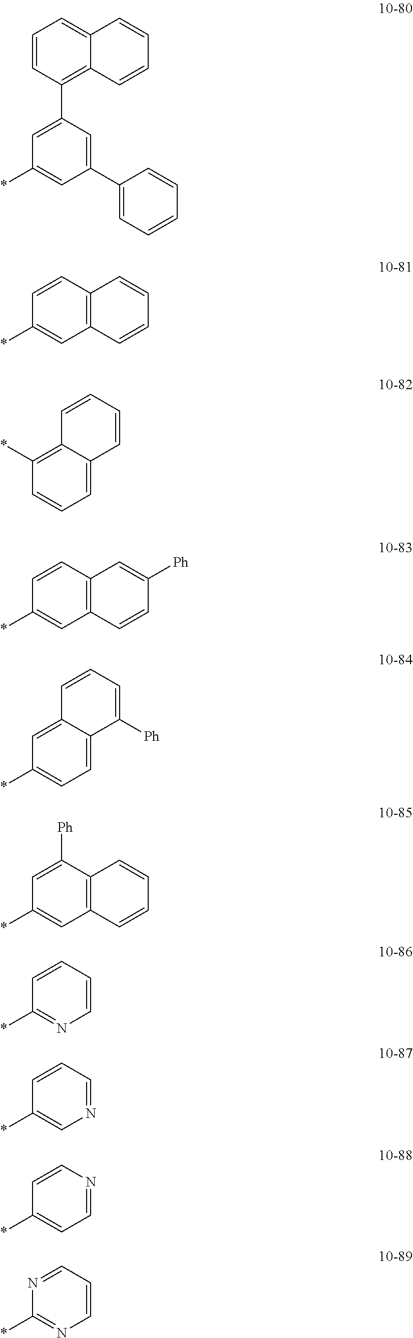

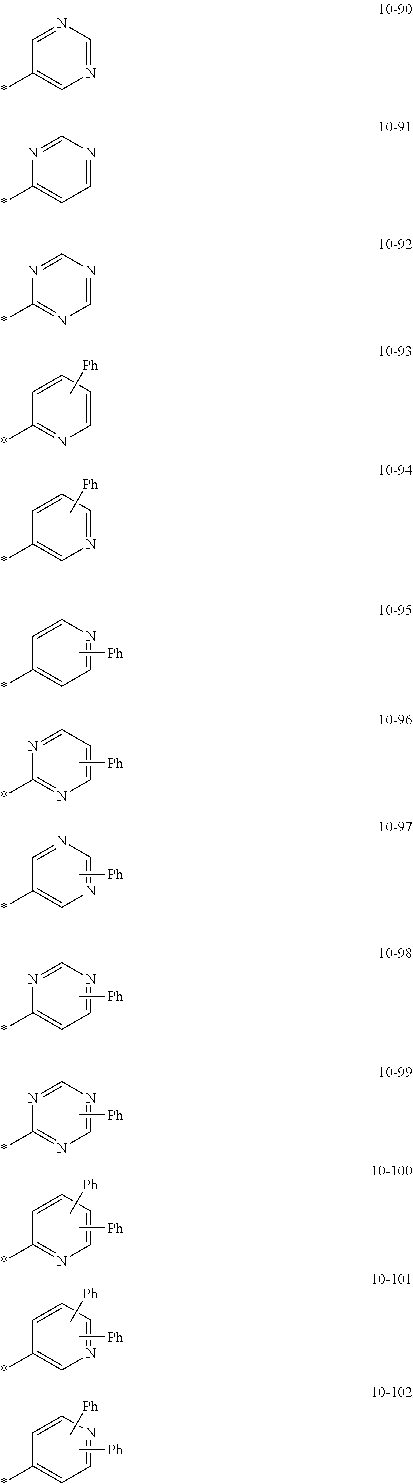

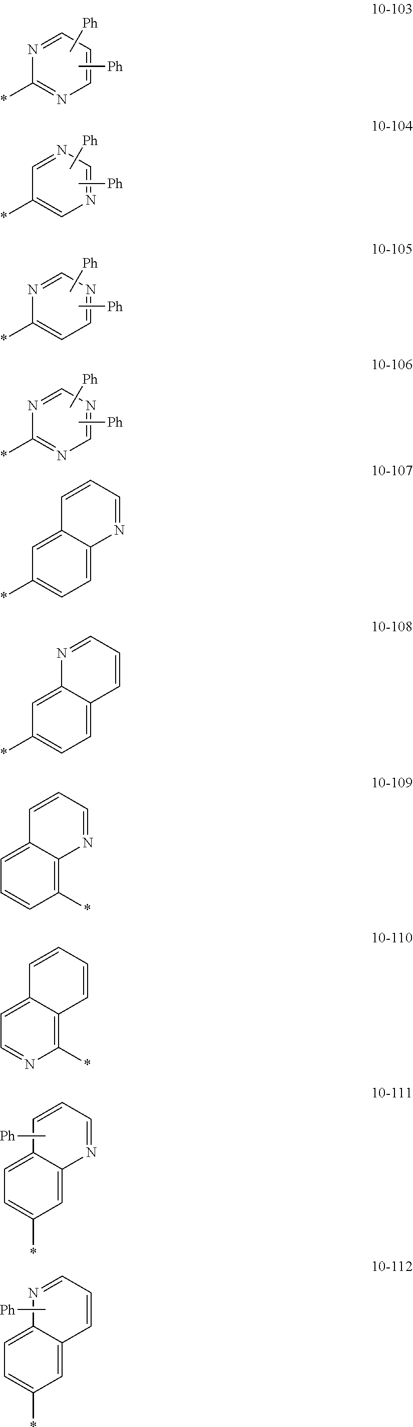

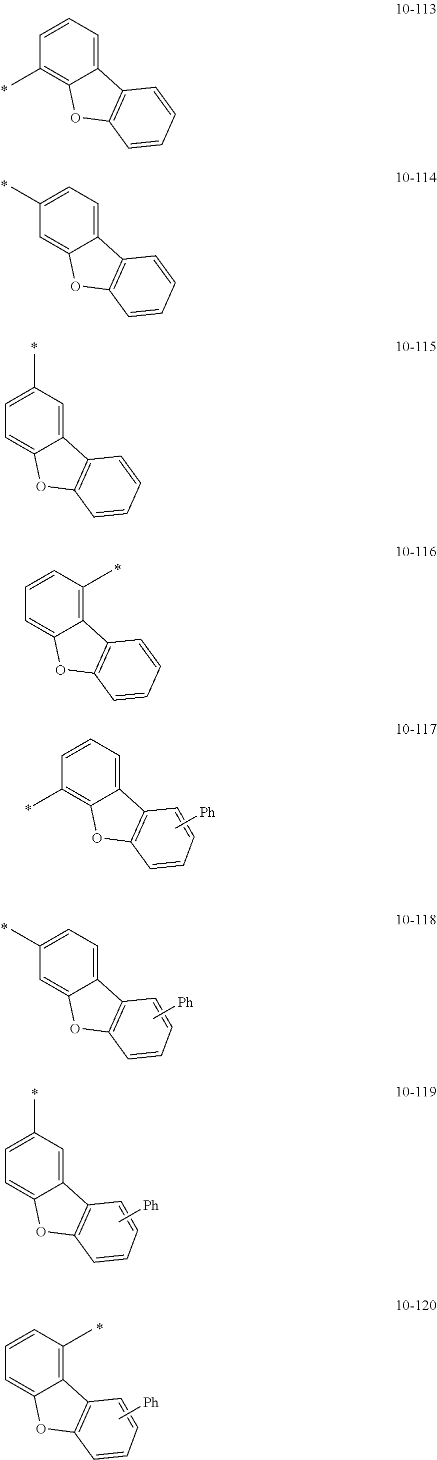

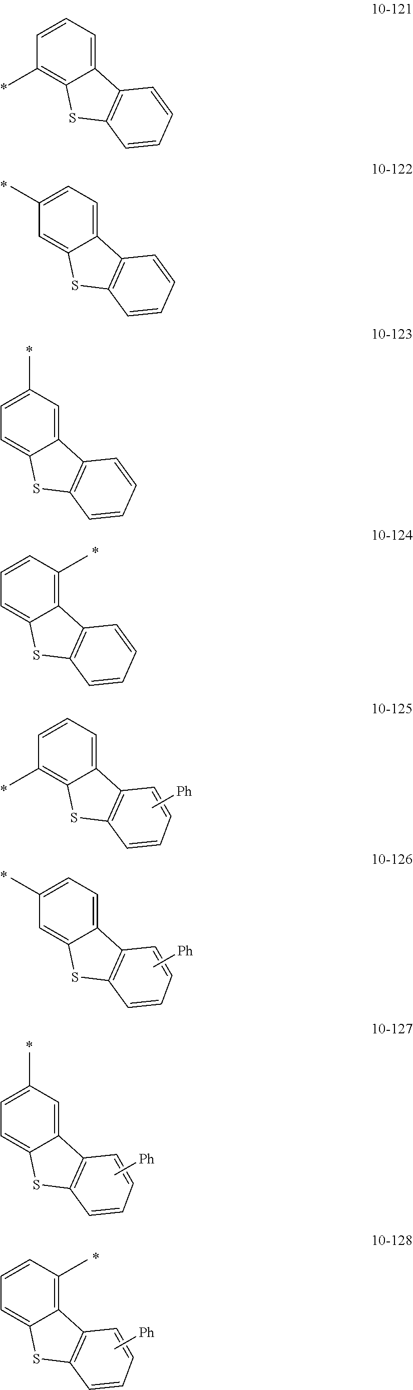

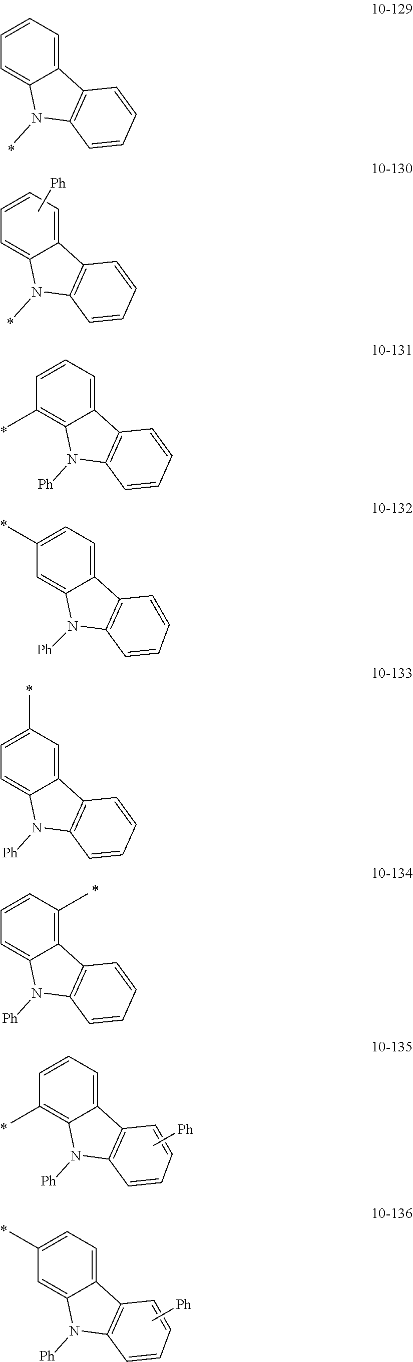

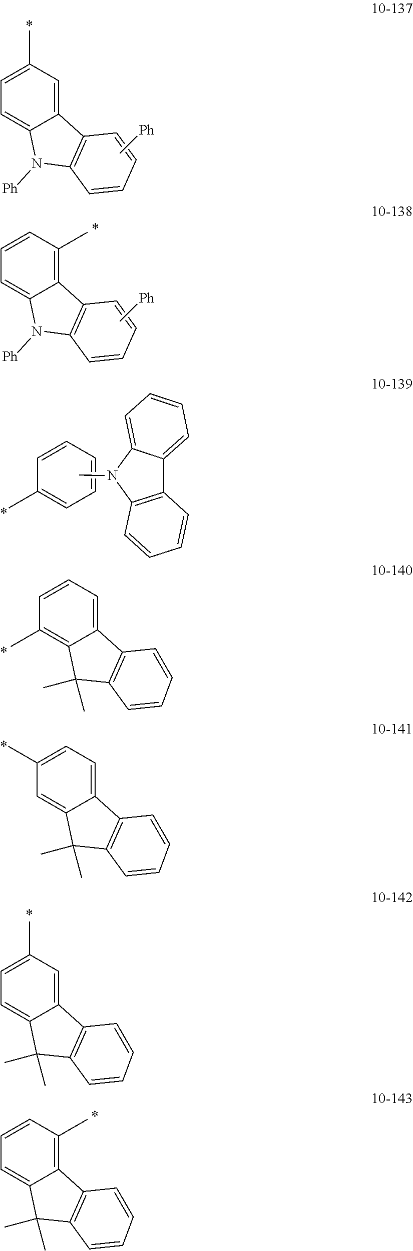

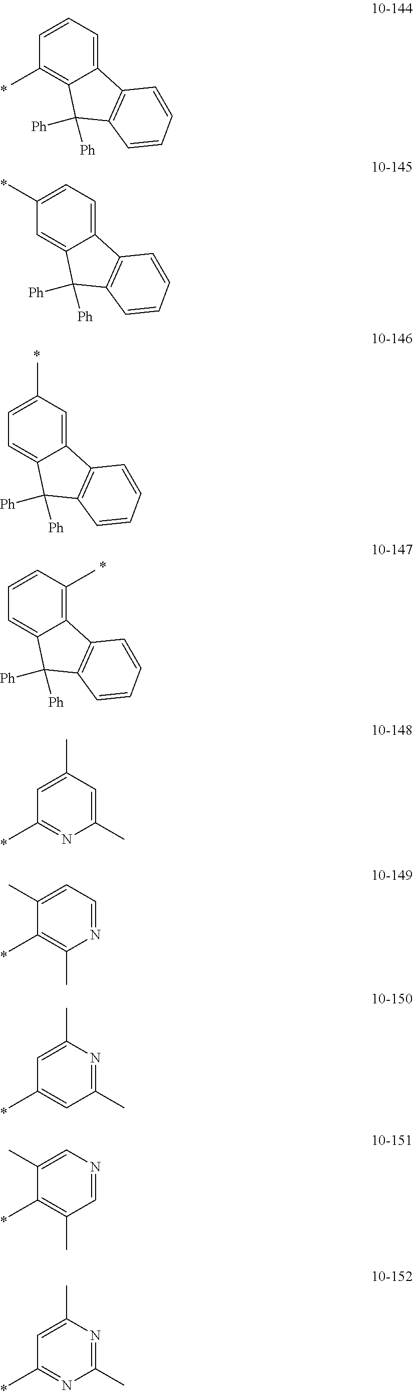

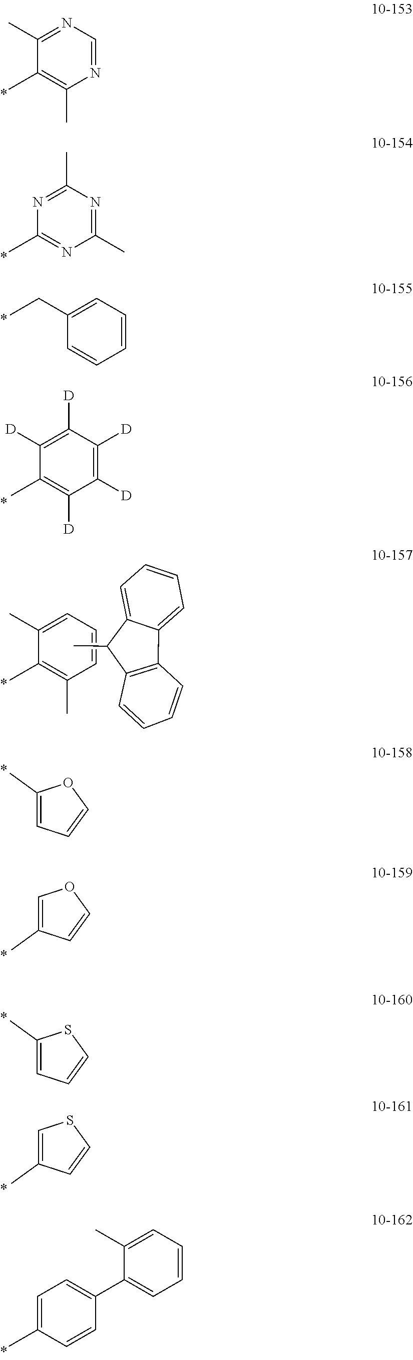

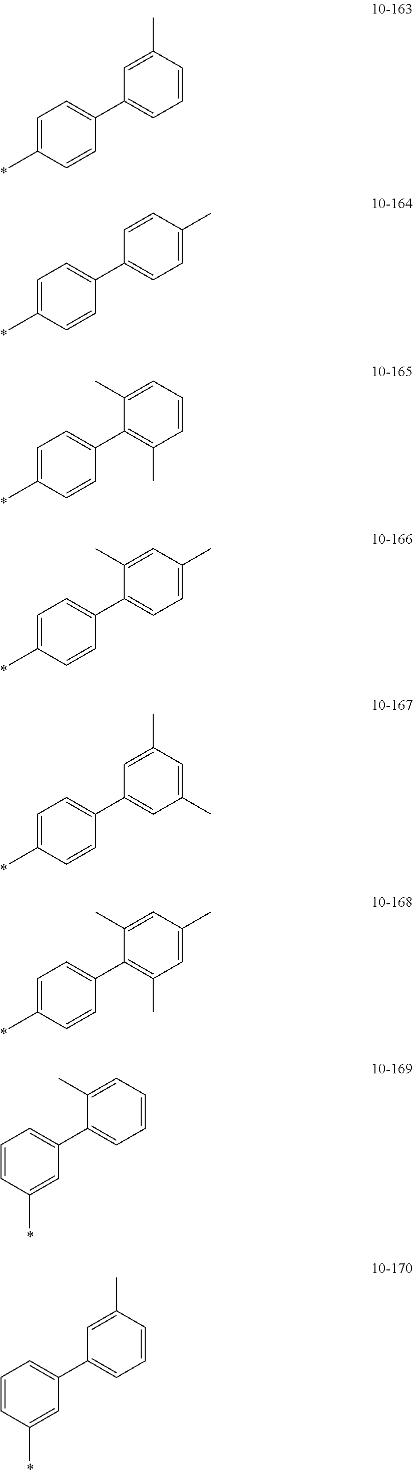

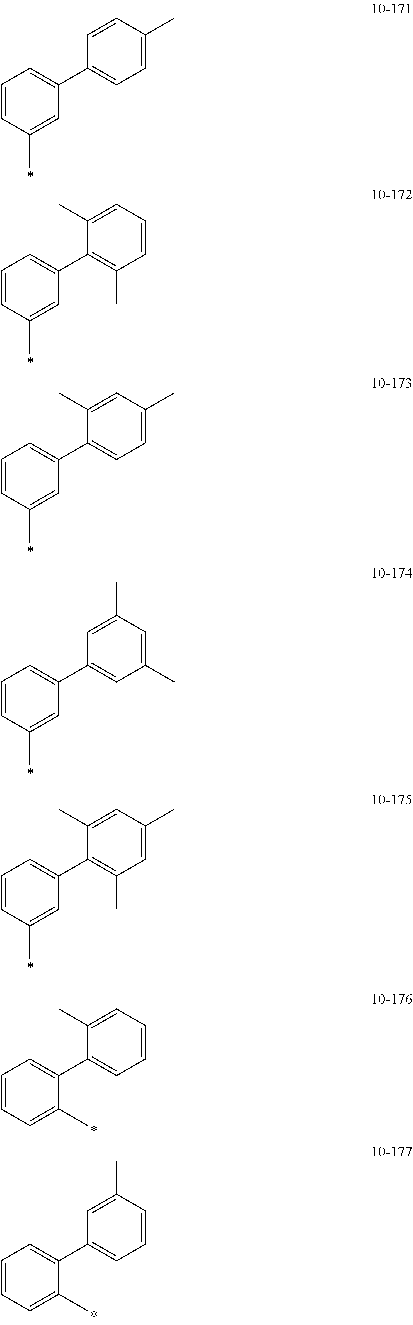

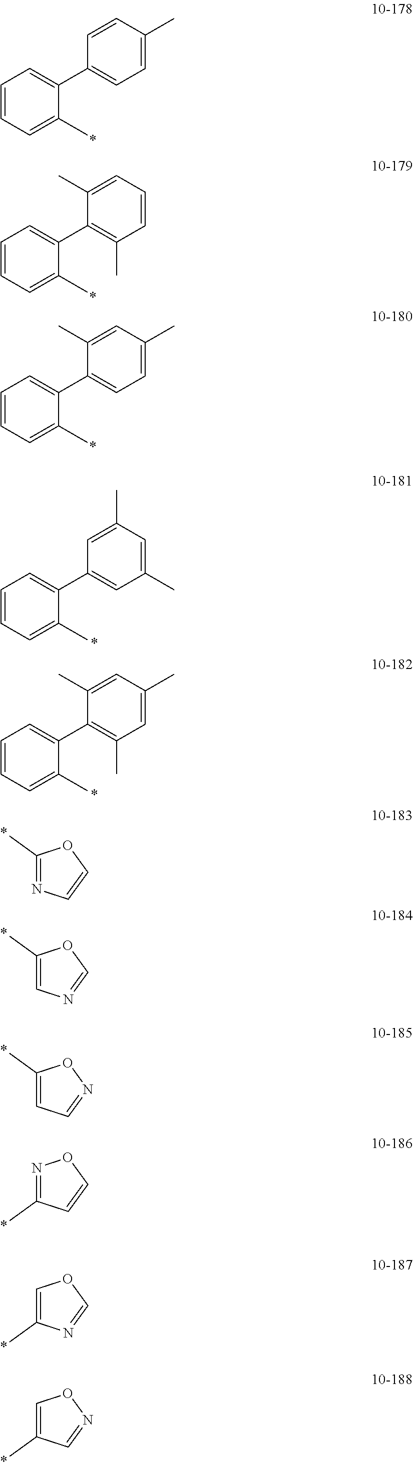

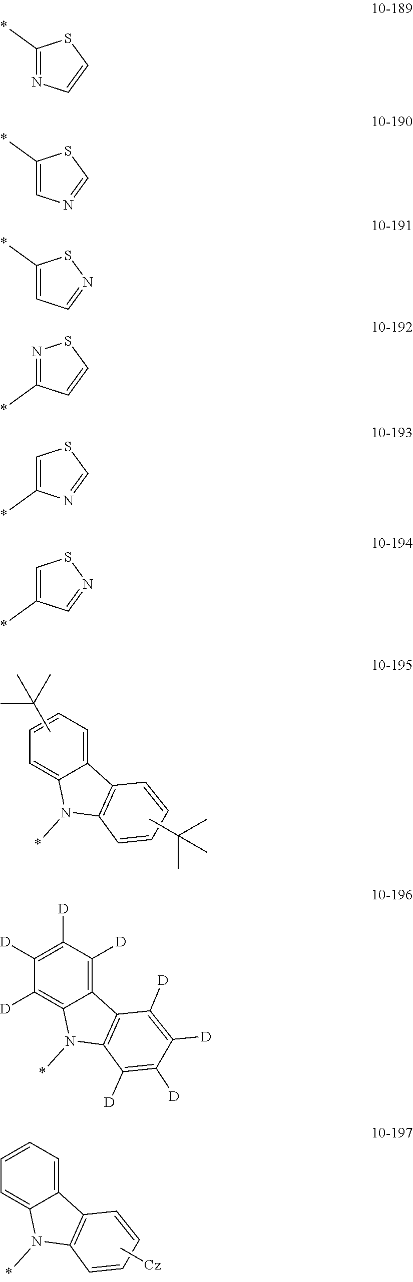

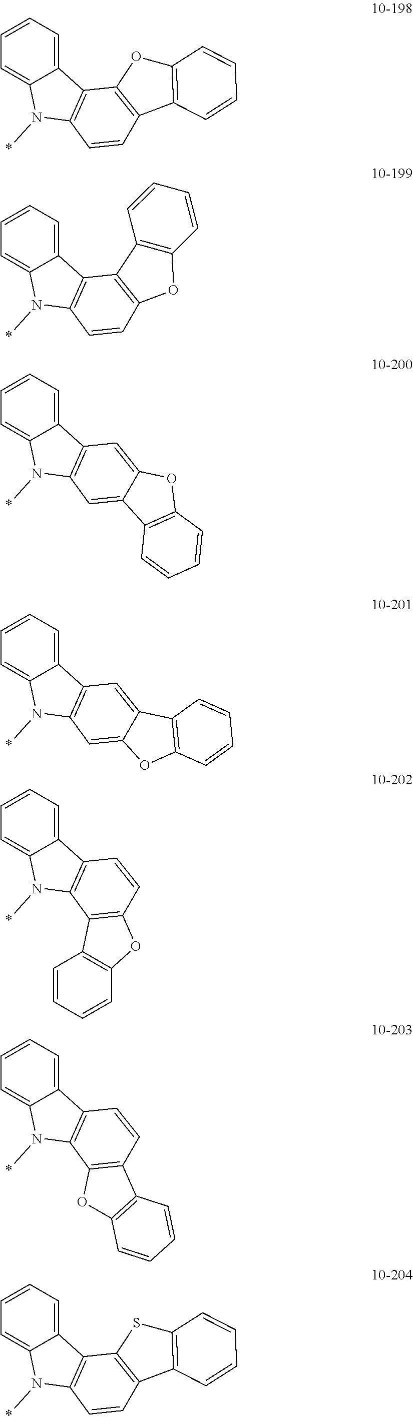

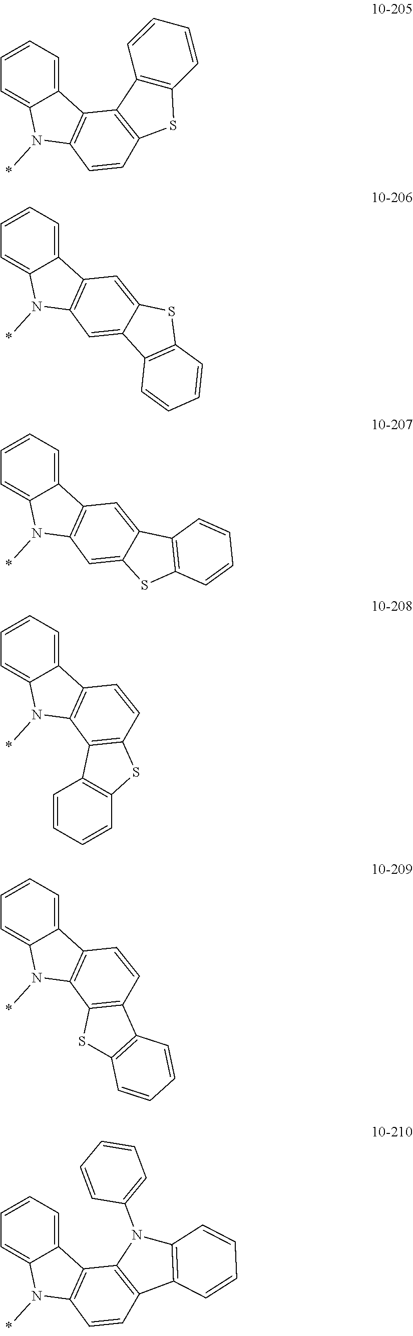

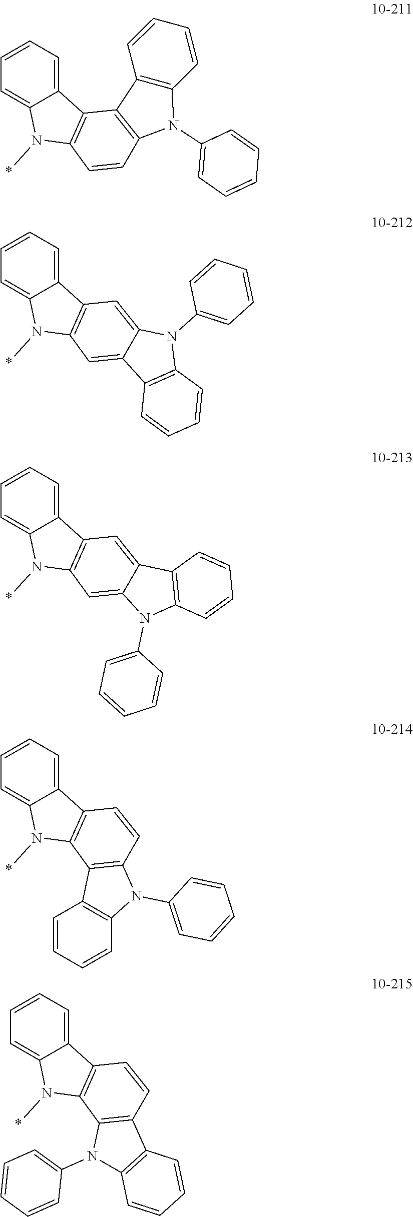

wherein, in Formulae 9-1 to 9-19 and 10-1 to 10-215, * indicates a binding site to an adjacent atom, “Ph” represents a phenyl group “TMS” represents a trimethylsilyl group, and Cz represents a carbazolyl group.

a1, a2, and a5 in Formula 1 may each independently be an integer of 1 to 10. When a1 is an integer of 2 or more, two or more of R₁(s) may be identical to or different from each other; when a2 is an integer of 2 or more, two or more of R₂(s) may be identical to or different from each other; and when a5 is an integer of 2 or more, two or more of R₅(s) may be identical to or different from each other.

In an embodiment, in Formula 1, at least one of R₁(s) in the number of a1 and R₂(s) in the number of a2 may be an electron donating group connected via (L₁)_{b1}, L₁ may be a substituted or unsubstituted C₅-C₆₀ carbocyclic group or a substituted or unsubstituted C₁-C₆₀ heterocyclic group, and b1 may be 0, 1, or 2.

For example, at least one of R₁(s) in the number of a1 and R₂(s) in the number of a2 in Formula 1 may be a group represented by Formula 2A or 2B:

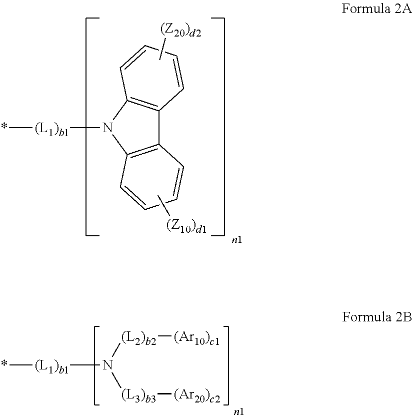

wherein, in Formulae 2A and 2B,

L₁ to L₃, Ar₁₀ and Ar₂₀ may each independently be a substituted or unsubstituted C₅-C₆₀ carbocyclic group or a substituted or unsubstituted C₁-C₆₀ heterocyclic group,

b1 to b3 may each independently be 0, 1, or 2,

c1 and c2 may each independently be an integer from 1 to 5,

Z₁₀ and Z₂₀ may each independently be a deuterium, —F, —Cl, —Br, —I, —SF₅, a hydroxyl group, a cyano group, a nitro group, a C₁-C₆₀ alkyl group, a C₁-C₆₀ alkoxy group, a cyclopentyl group, a cyclohexyl group, a cyclopentenyl group, a cyclohexenyl group, a cycloheptenyl group, a phenyl group, a naphthyl group, a pentalenyl group, an indenyl group, an azulenyl group, a heptalenyl group, an acenaphthyl group, a fluorenyl group, a phenalenyl group, a phenanthrenyl group, an anthracenyl group, a fluoranthenyl group, a triphenylenyl group, a pyrenyl group, a chrysenylenyl group, a naphthacenyl group, a picenyl group, a pyridinyl group, a pyrazinyl group, a pyrimidinyl group, a pyridazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, or a carbazolyl group,

d1 and d2 may each independently be an integer from 0 to 4,

n1 may be an integer from 1 to 3, and

* indicates a binding site to an adjacent atom.

In an embodiment, L₁ to L₃ in Formulae 2A and 2B may each independently be a phenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, or a chrysenylene group, each unsubstituted or substituted with deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a phenyl group, an indenyl group, a naphthyl group, an azulenyl group, a heptalenyl group, an acenaphthyl group, a fluorenyl group, a phenalenyl group, a phenanthrenyl group, an anthracenyl group, a fluoranthenyl group, a triphenylenyl group, a pyrenyl group, a chrysenylenyl, or any combination thereof.

For example, L₁ to L₃ may each independently be Formulae 3-1 to 3-32, and embodiments of the present disclosure are not limited thereto:

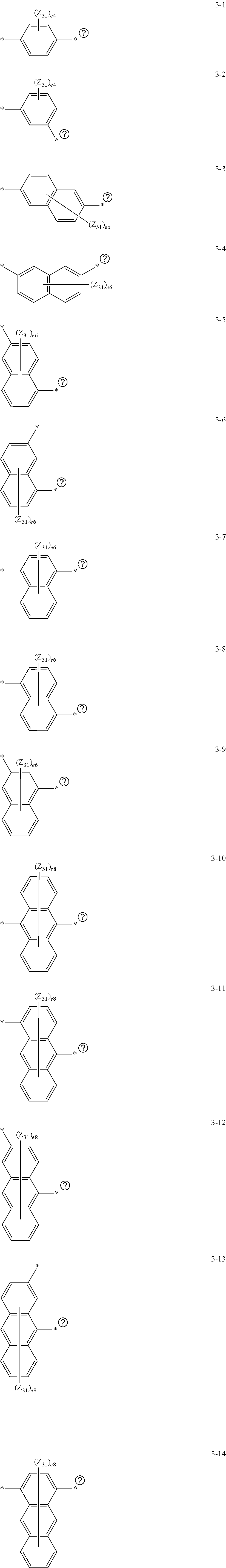

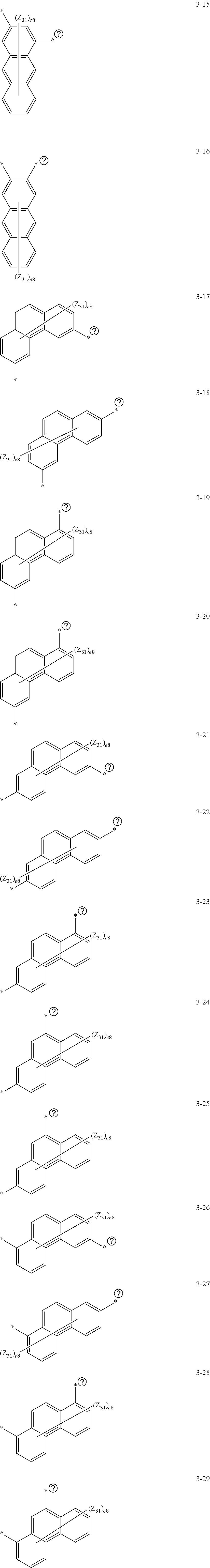

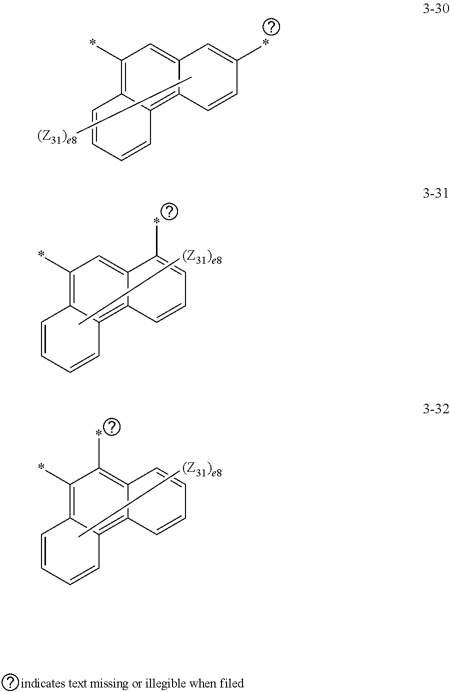

wherein, in Formulae 3-1 to 3-32,

Z₃₁ may be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₆-C₆₀ aryl alkyl group, or a C₁-C₀ heteroaryl group,

e4 may be an integer from 1 to 4,

e6 may be an integer from 1 to 6,

e8 may be an integer from 1 to 8, and

* and *′ each indicate a binding site to an adjacent atom.

In an embodiment, b1 to b3 in Formulae 2A and 2B may each independently be 0 or 1.

In an embodiment, Ar₁₀ and Ar₂₀ may each independently be a cyclopentyl, a cyclohexyl group, a cyclopentenyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, or a dibenzothiophenyl group, each unsubstituted or substituted with deuterium, —F, a cyano group, a C₁-C₂₀ alkyl group, a C₁-C₂₀ alkoxy group, a cyclopentyl group, a cyclohexyl group, a cyclopentenyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof.

In an embodiment, one or two of R₁(s) in the number of a1 and R₂(s) in the number of a2 may be a group represented by Formula 2A or 2B, and embodiments of the present disclosure are not limited thereto.

A substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, substituted monovalent non-aromatic condensed polycyclic group, or substituted monovalent non-aromatic condensed heteropolycyclic group may be:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or any combination thereof;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or any combination thereof;

—N(Q₃₁)(Q₃₂), —Si(Q₃₃)(Q₃₄)(Q₃₅), —B(Q₃₆)(Q₃₇), or —P(═O)(Q₃₈)(Q₃₉); or

any combination thereof,

wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₁-C₆₀ alkyl group which is unsubstituted or substituted with deuterium, a C₁-C₆₀ alkyl group, a C₆-C₆₀ aryl group, or any combination thereof, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group which is unsubstituted or substituted with deuterium, a C₁-C₆₀ alkyl group, a C₆-C₆₀ aryl group, or any combination thereof, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group.

In an embodiment, the condensed cyclic compound may be represented by Formula 1A-1 or 1B-1:

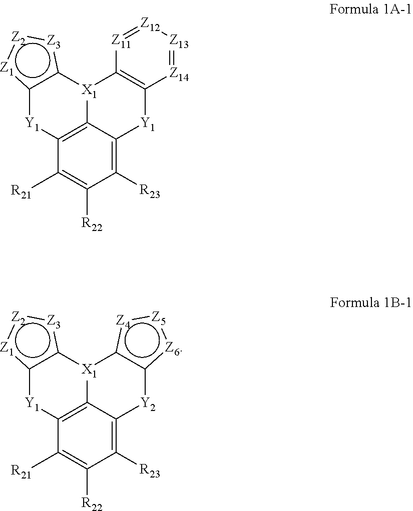

In regard to Formulae 1A-1 and 1B-1, X₁, Y₁, Y₂, Z₁₁ to Z₁₄, and Z₁ to Z₆ are each the same as described in the present specification, and R₂₁ to R₂₃ are each the same as described in connection with R₂.

In an embodiment, at least one of R₁₃ and R₂₂ in Formula 1A-1 or R₂₂ in Formula 1B-1 may be a group represented by Formula 2A or 2B. For example, R₁₃ or R₂₂ in Formula 1A-1 may be a group represented by Formula 2A or 2B. In an embodiment, R₁₃ and R₂₂ in Formula 1A-1 may each be a group represented by Formula 2A or 2B.

For example, the condensed cyclic compound represented by Formula 1 may be one of Compounds 1 to 2363, and embodiments of the present disclosure are not limited thereto:

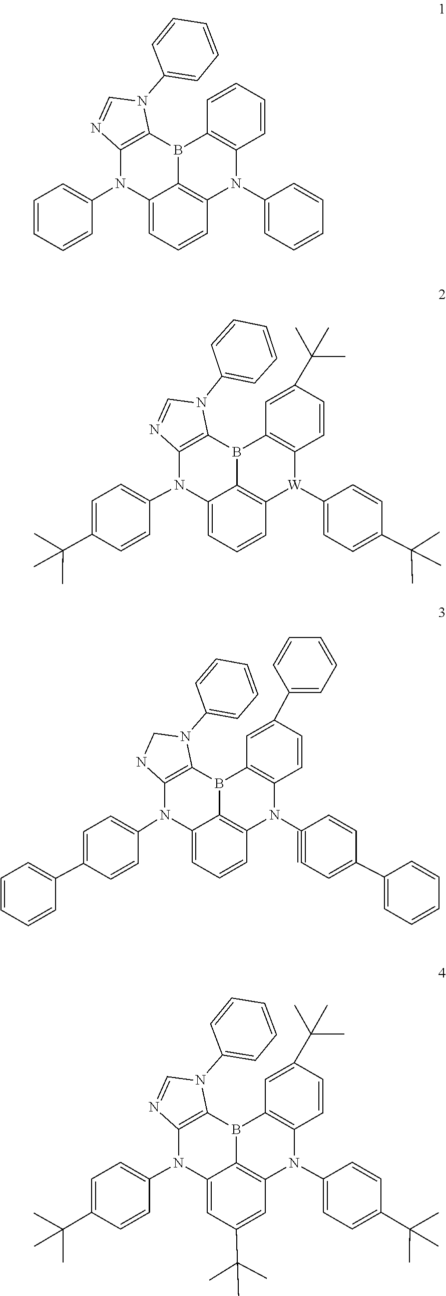

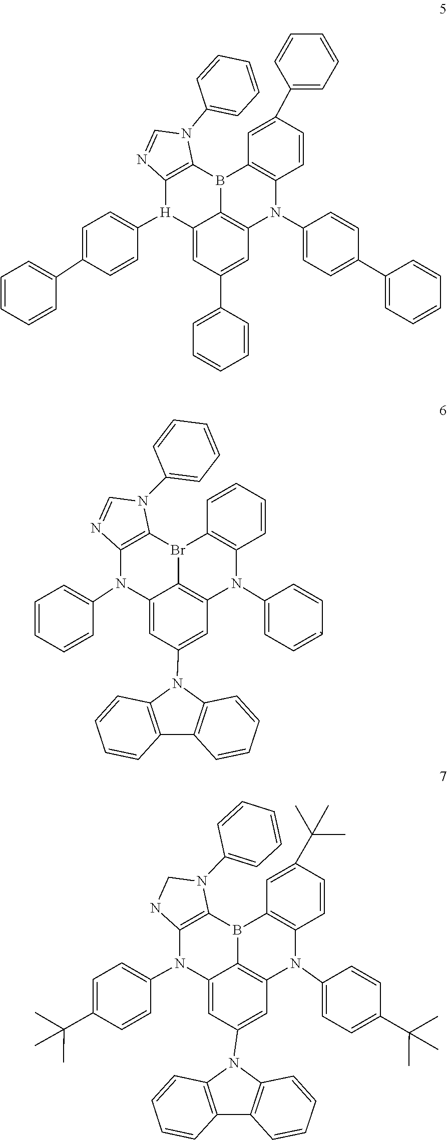

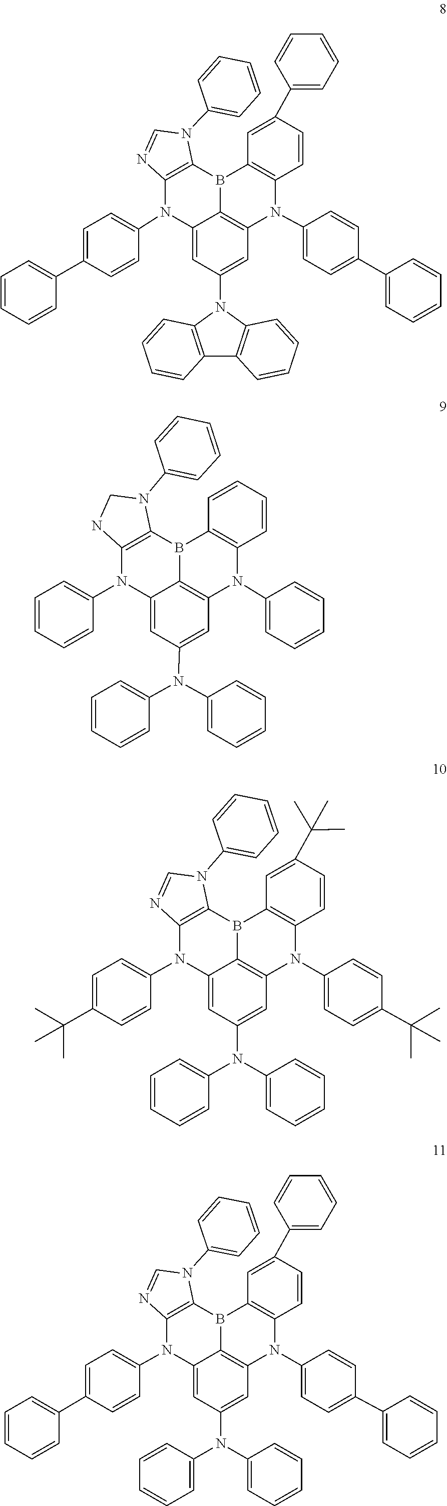

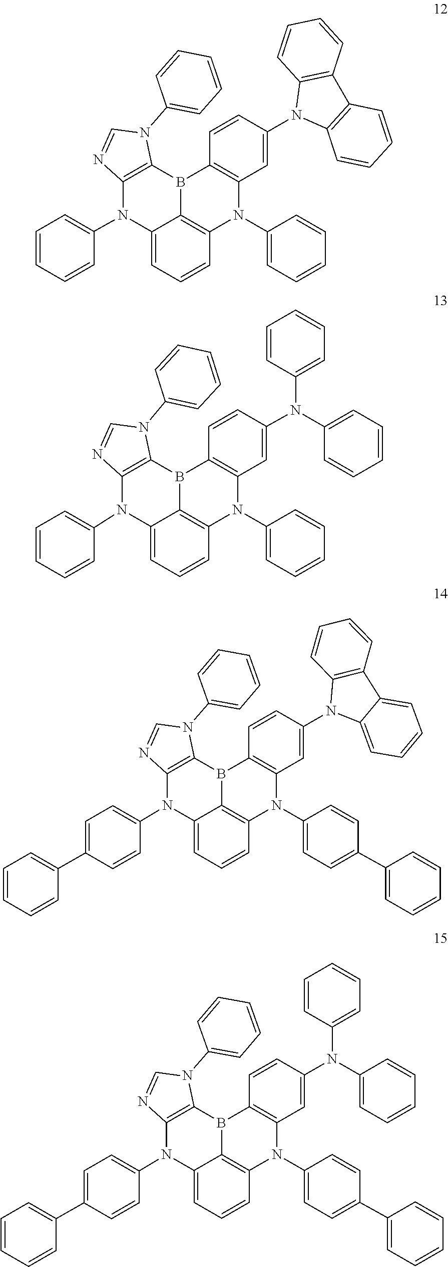

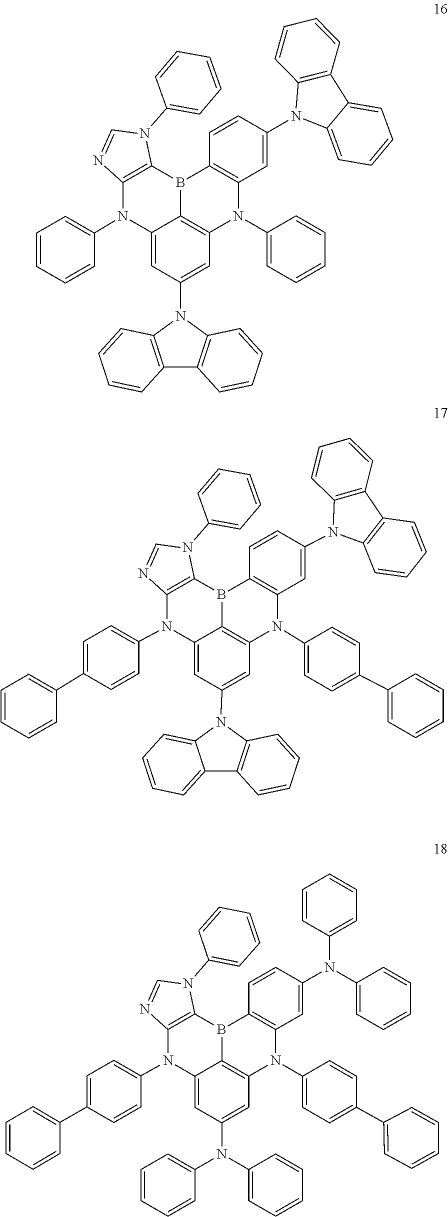 [SMILES: c1ccc(N2c3cc(-n4c5ccccc5c5ccccc54)ccc3B3c4c2cc(-n2c5ccccc5c5ccccc52)cc4N(c2ccccc2)c2ncn(-c4ccccc4)c23)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3ncn(-c5ccccc5)c34)cc2)cc1 ; c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3ncn(-c5ccccc5)c34)cc2)cc1]

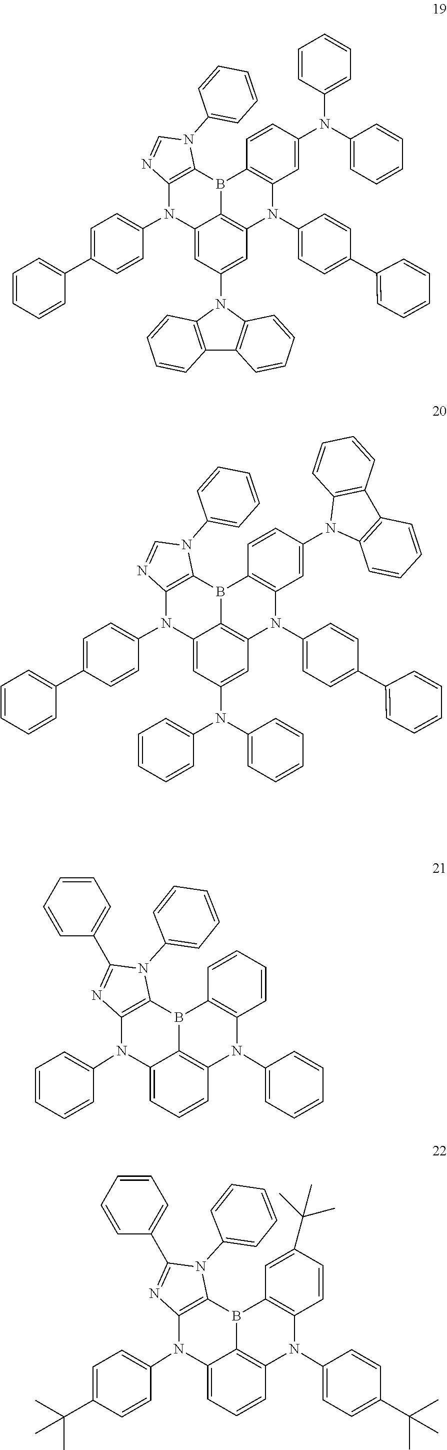 [SMILES: c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3ncn(-c5ccccc5)c34)cc2)cc1 ; c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3ncn(-c5ccccc5)c34)cc2)cc1 ; c1ccc(-c2nc3c(n2-c2ccccc2)B2c4ccccc4N(c4ccccc4)c4cccc(c42)N3c2ccccc2)cc1 ; CC(C)(C)c1ccc(N2c3ccc(C(C)(C)C)cc3B3c4c2cccc4N(c2ccc(C(C)(C)C)cc2)c2nc(-c4ccccc4)n(-c4ccccc4)c23)cc1]

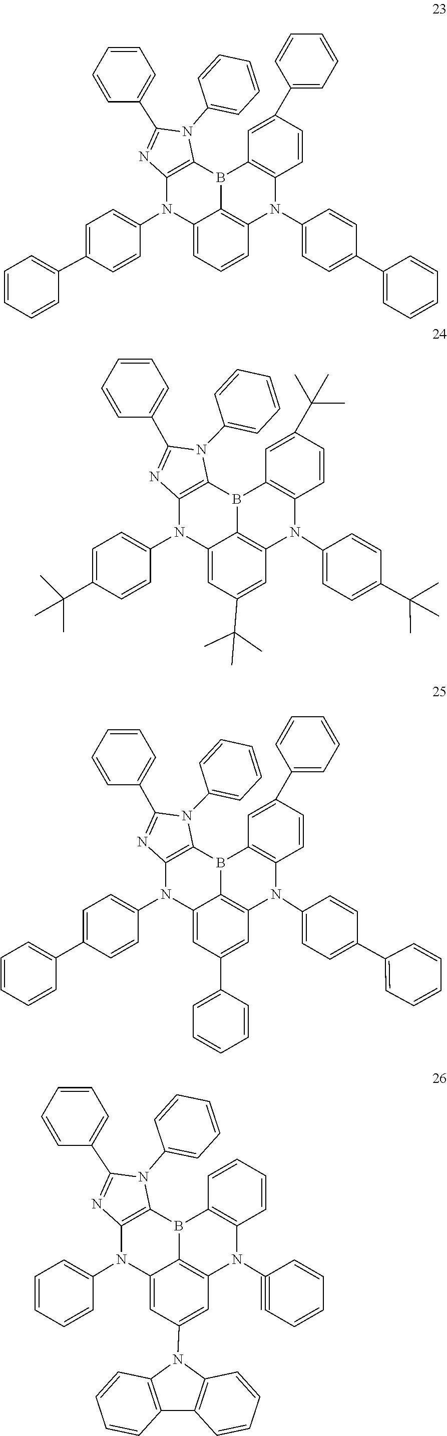

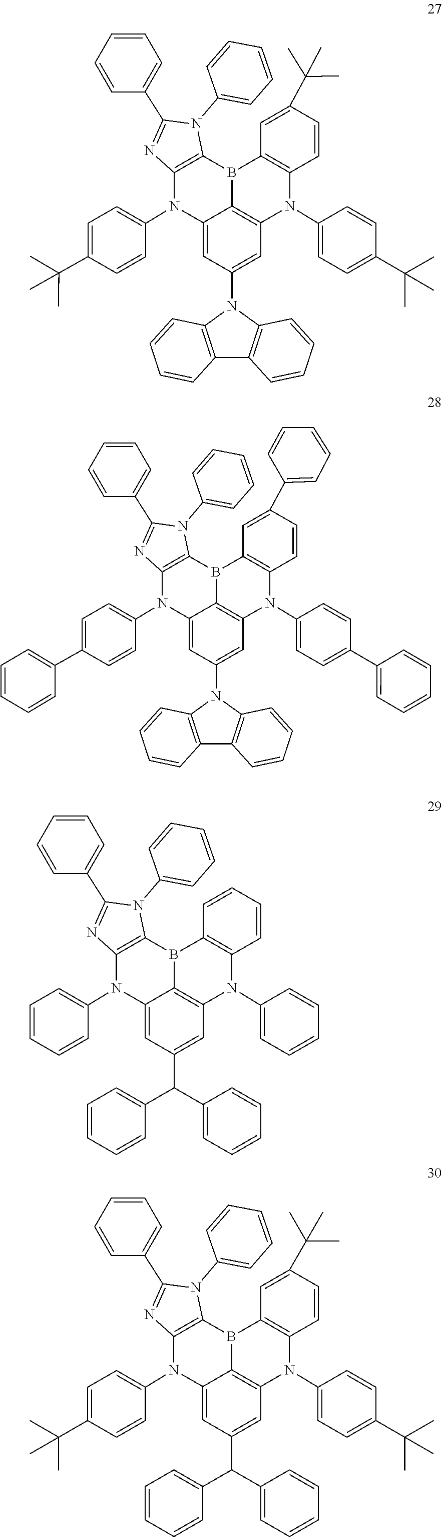

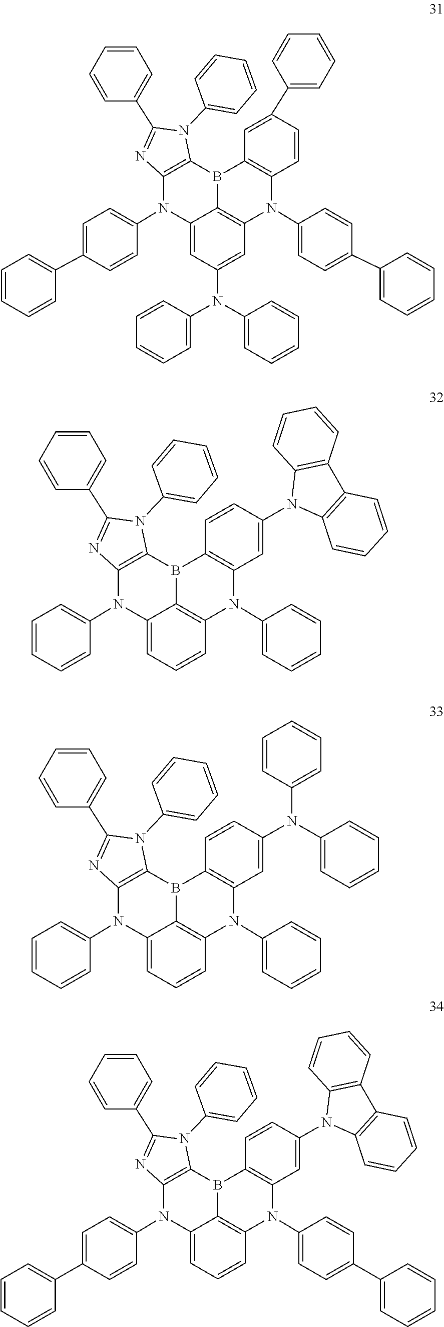 [SMILES: c1ccc(-c2ccc(N3c4ccc(-c5ccccc5)cc4B4c5c3cc(N(c3ccccc3)c3ccccc3)cc5N(c3ccc(-c5ccccc5)cc3)c3nc(-c5ccccc5)n(-c5ccccc5)c34)cc2)cc1 ; c1ccc(-c2nc3c(n2-c2ccccc2)B2c4ccc(-n5c6ccccc6c6ccccc65)cc4N(c4ccccc4)c4cccc(c42)N3c2ccccc2)cc1 ; c1ccc(-c2nc3c(n2-c2ccccc2)B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5c3cccc5N(c3ccc(-c5ccccc5)cc3)c3nc(-c5ccccc5)n(-c5ccccc5)c34)cc2)cc1]

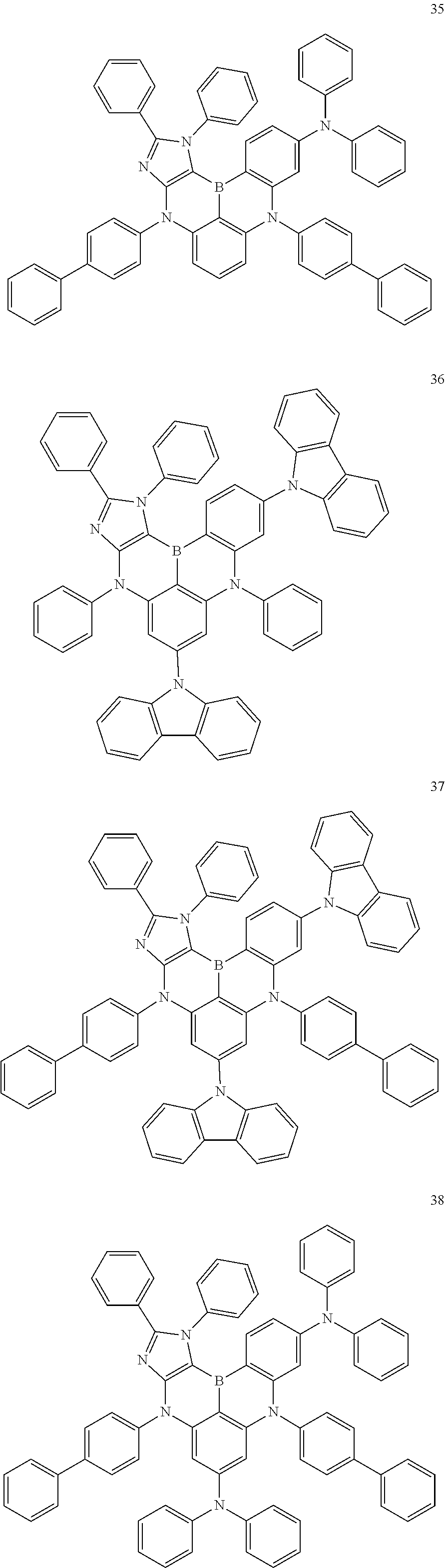

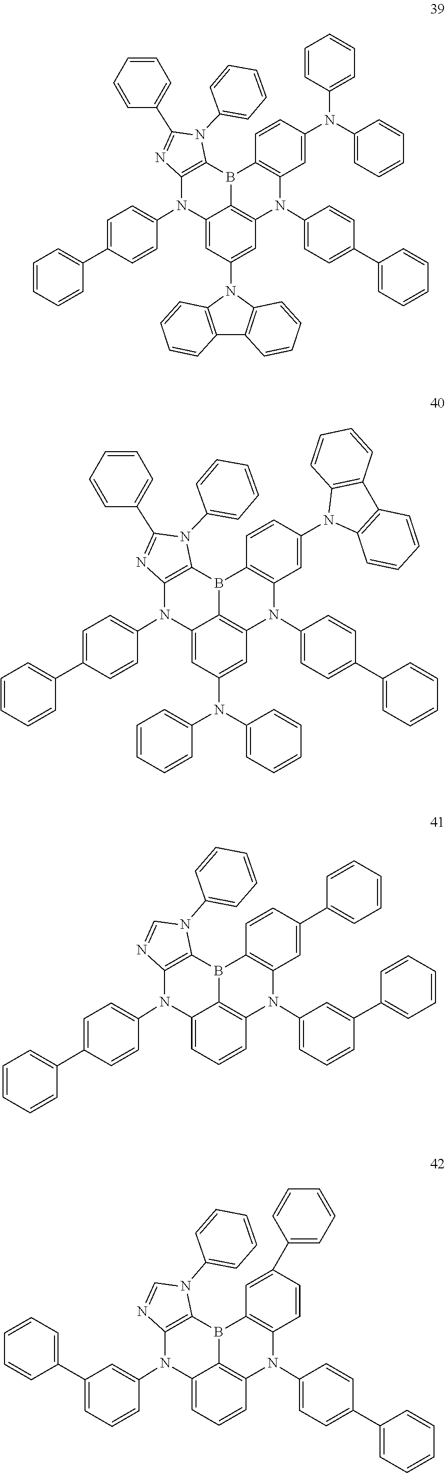 [SMILES: c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3nc(-c5ccccc5)n(-c5ccccc5)c34)cc2)cc1 ; c1ccc(-c2ccc(N3c4cccc5c4B(c4ccc(-c6ccccc6)cc4N5c4cccc(-c5ccccc5)c4)c4c3ncn4-c3ccccc3)cc2)cc1 ; c1ccc(-c2ccc(N3c4ccc(-c5ccccc5)cc4B4c5c3cccc5N(c3cccc(-c5ccccc5)c3)c3ncn(-c5ccccc5)c34)cc2)cc1]

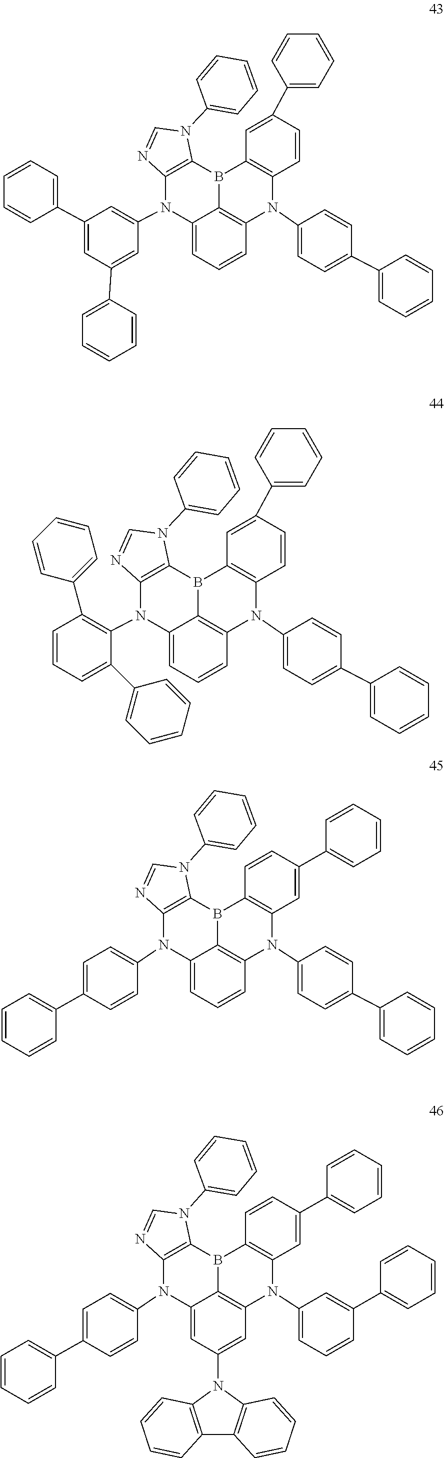 [SMILES: c1ccc(-c2ccc(N3c4ccc(-c5ccccc5)cc4B4c5c3cccc5N(c3cc(-c5ccccc5)cc(-c5ccccc5)c3)c3ncn(-c5ccccc5)c34)cc2)cc1 ; c1ccc(-c2ccc(N3c4ccc(-c5ccccc5)cc4B4c5c3cccc5N(c3c(-c5ccccc5)cccc3-c3ccccc3)c3ncn(-c5ccccc5)c34)cc2)cc1 ; c1ccc(-c2ccc(N3c4cc(-c5ccccc5)ccc4B4c5c3cccc5N(c3ccc(-c5ccccc5)cc3)c3ncn(-c5ccccc5)c34)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c3ncn4-c3ccccc3)cc2)cc1]

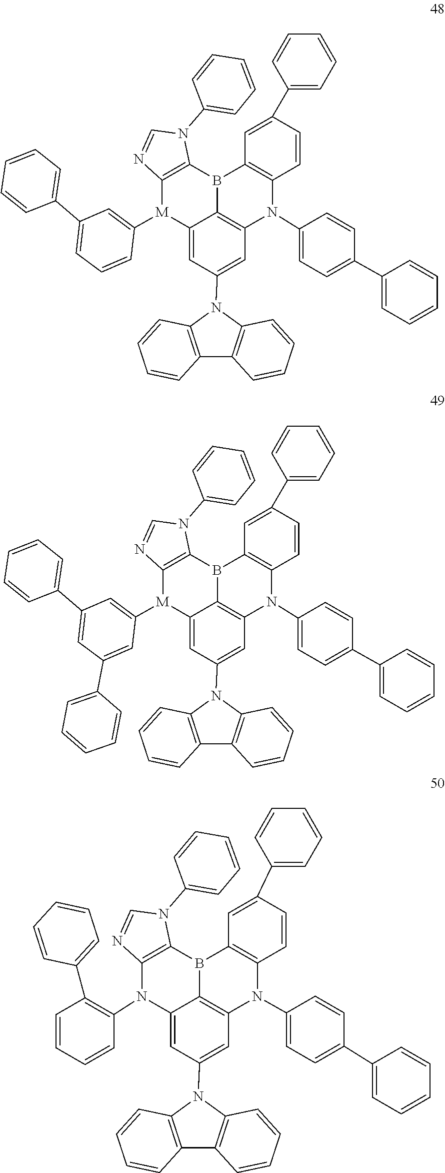

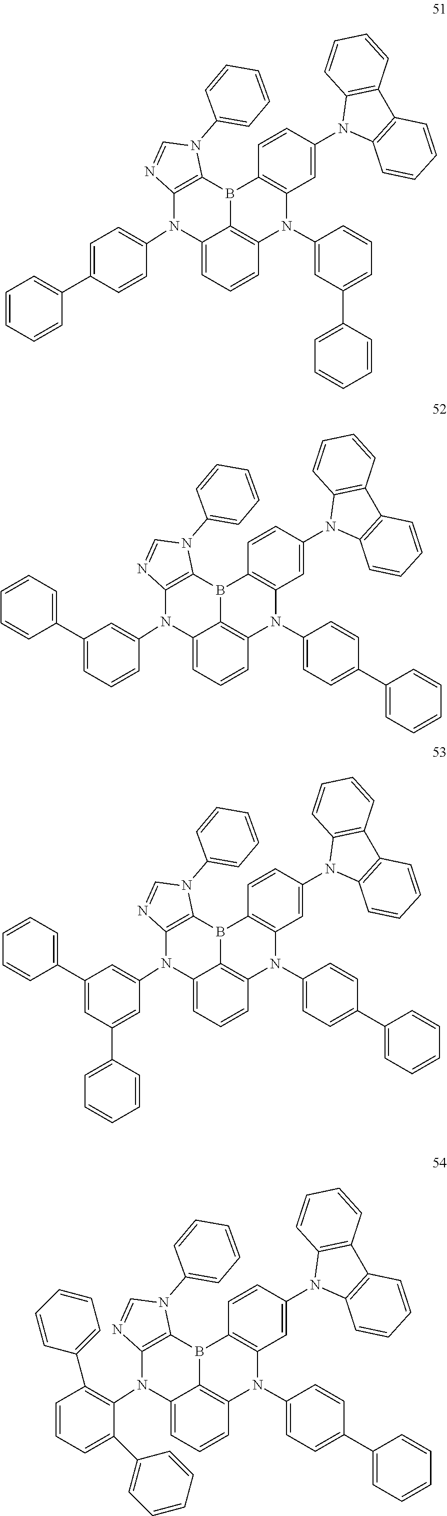 [SMILES: c1ccc(-c2ccc(N3c4cccc5c4B(c4ccc(-n6c7ccccc7c7ccccc76)cc4N5c4cccc(-c5ccccc5)c4)c4c3ncn4-c3ccccc3)cc2)cc1 ; c1ccc(-c2ccc(N3c4cc(-n5c6ccccc6c6ccccc65)ccc4B4c5c3cccc5N(c3cccc(-c5ccccc5)c3)c3ncn(-c5ccccc5)c34)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3ncn(-c5ccccc5)c34)cc2)cc1 ; c1ccc(-c2ccc(N3c4cc(-n5c6ccccc6c6ccccc65)ccc4B4c5c3cccc5N(c3c(-c5ccccc5)cccc3-c3ccccc3)c3ncn(-c5ccccc5)c34)cc2)cc1]

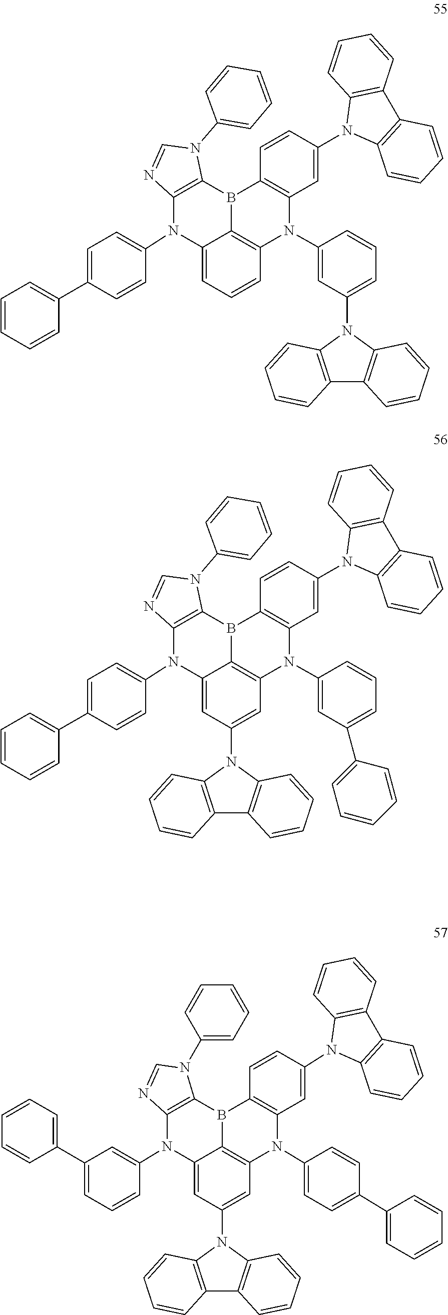 [SMILES: c1ccc(-c2ccc(N3c4cccc5c4B(c4ccc(-n6c7ccccc7c7ccccc76)cc4N5c4cccc(-n5c6ccccc6c6ccccc65)c4)c4c3ncn4-c3ccccc3)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3ncn4-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3ncn(-c5ccccc5)c34)cc2)cc1]

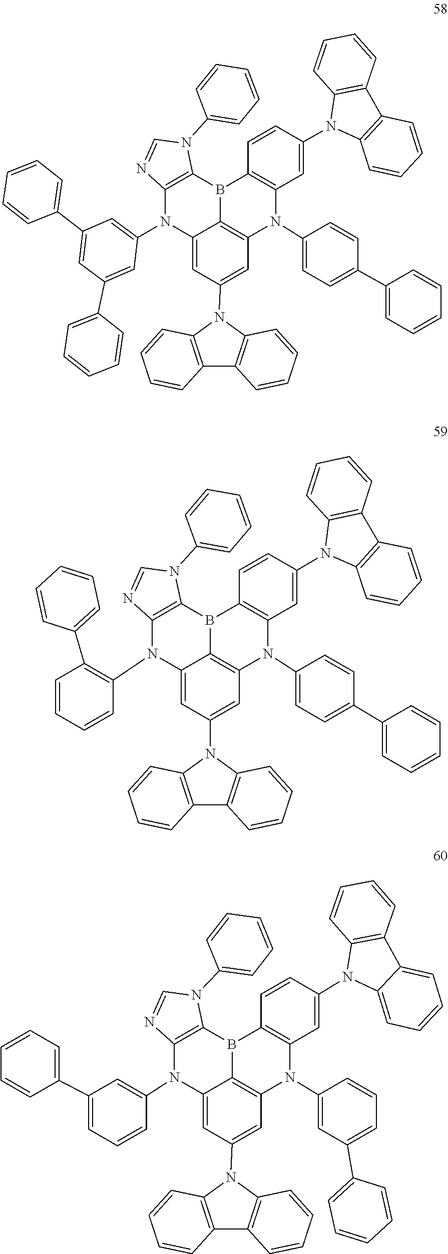 [SMILES: c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3ncn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3ncn(-c5ccccc5)c34)cc2)cc1 ; c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3ncn(-c5ccccc5)c34)c2)cc1]

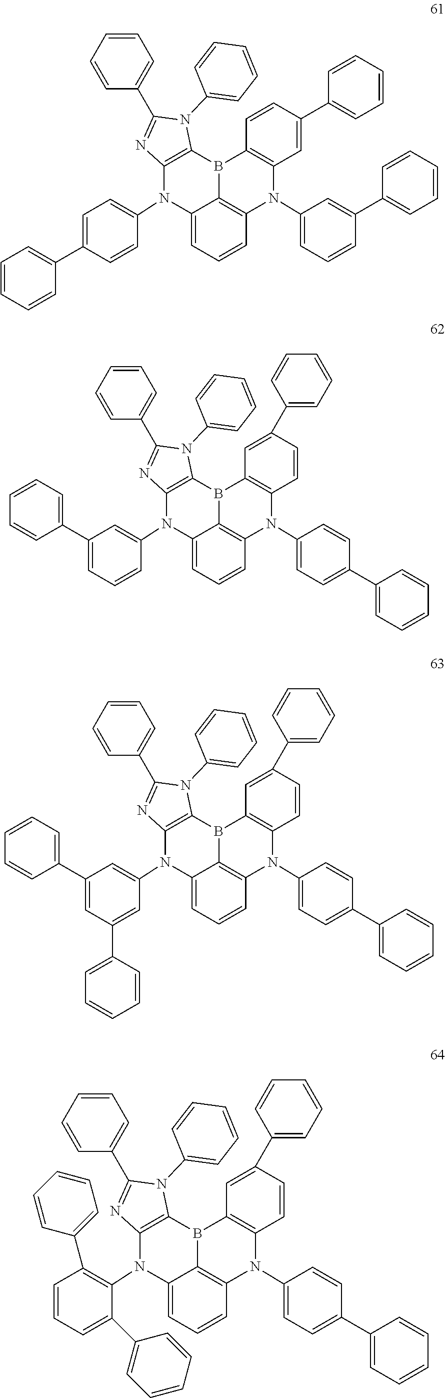 [SMILES: c1ccc(-c2ccc(N3c4cccc5c4B(c4ccc(-c6ccccc6)cc4N5c4cccc(-c5ccccc5)c4)c4c3nc(-c3ccccc3)n4-c3ccccc3)cc2)cc1 ; c1ccc(-c2ccc(N3c4ccc(-c5ccccc5)cc4B4c5c3cccc5N(c3cccc(-c5ccccc5)c3)c3nc(-c5ccccc5)n(-c5ccccc5)c34)cc2)cc1 ; c1ccc(-c2ccc(N3c4ccc(-c5ccccc5)cc4B4c5c3cccc5N(c3cc(-c5ccccc5)cc(-c5ccccc5)c3)c3nc(-c5ccccc5)n(-c5ccccc5)c34)cc2)cc1 ; c1ccc(-c2ccc(N3c4ccc(-c5ccccc5)cc4B4c5c3cccc5N(c3c(-c5ccccc5)cccc3-c3ccccc3)c3nc(-c5ccccc5)n(-c5ccccc5)c34)cc2)cc1]

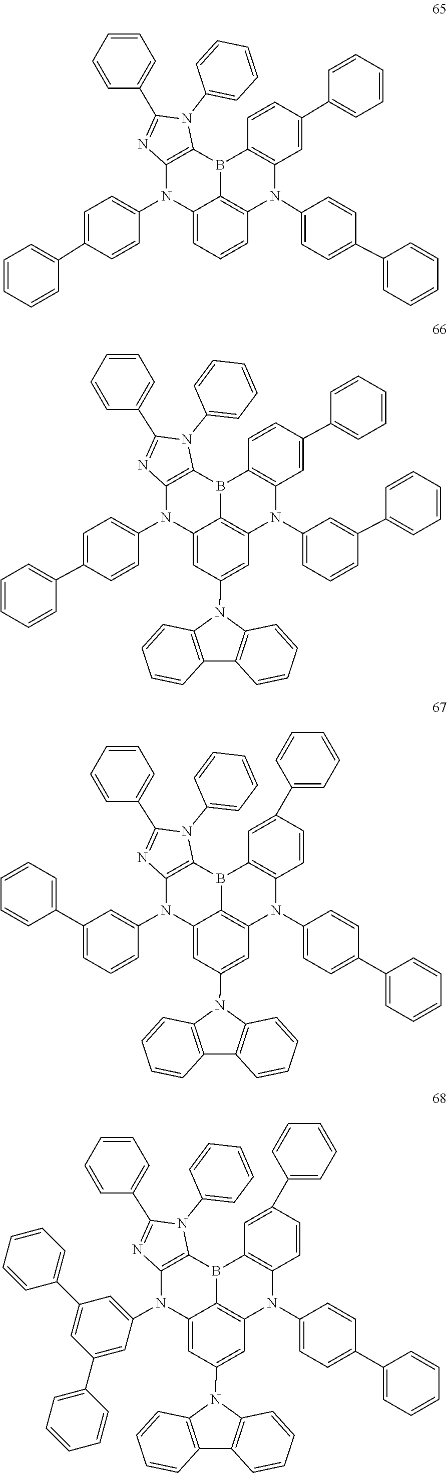

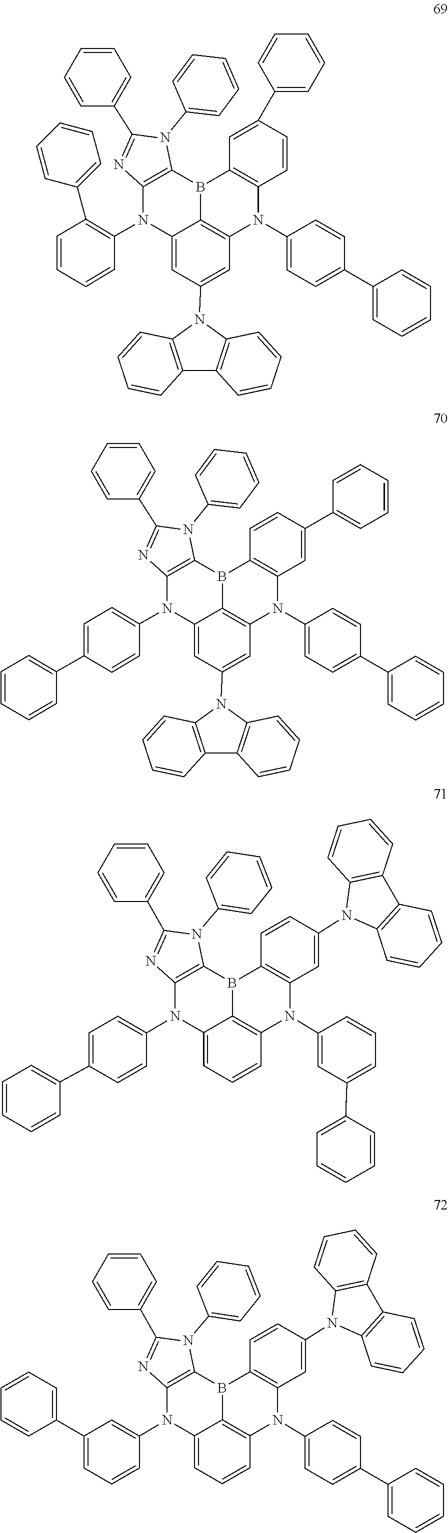 [SMILES: c1ccc(-c2ccc(N3c4ccc(-c5ccccc5)cc4B4c5c3cc(-n3c6ccccc6c6ccccc63)cc5N(c3ccccc3-c3ccccc3)c3nc(-c5ccccc5)n(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; c1ccc(-c2ccc(N3c4cccc5c4B(c4ccc(-n6c7ccccc7c7ccccc76)cc4N5c4cccc(-c5ccccc5)c4)c4c3nc(-c3ccccc3)n4-c3ccccc3)cc2)cc1 ; c1ccc(-c2ccc(N3c4cc(-n5c6ccccc6c6ccccc65)ccc4B4c5c3cccc5N(c3cccc(-c5ccccc5)c3)c3nc(-c5ccccc5)n(-c5ccccc5)c34)cc2)cc1]

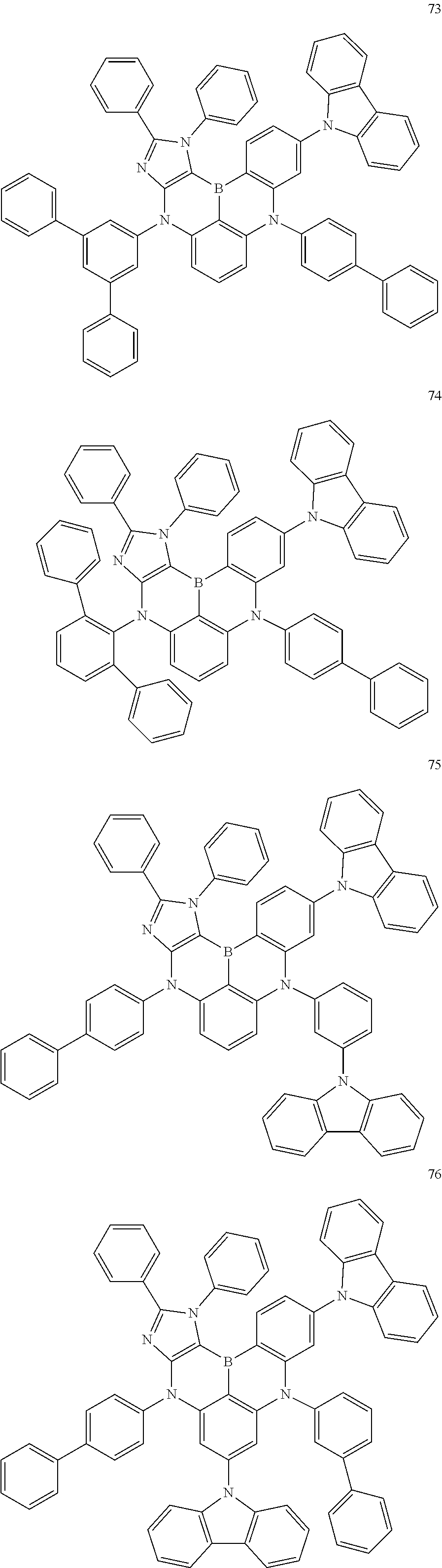

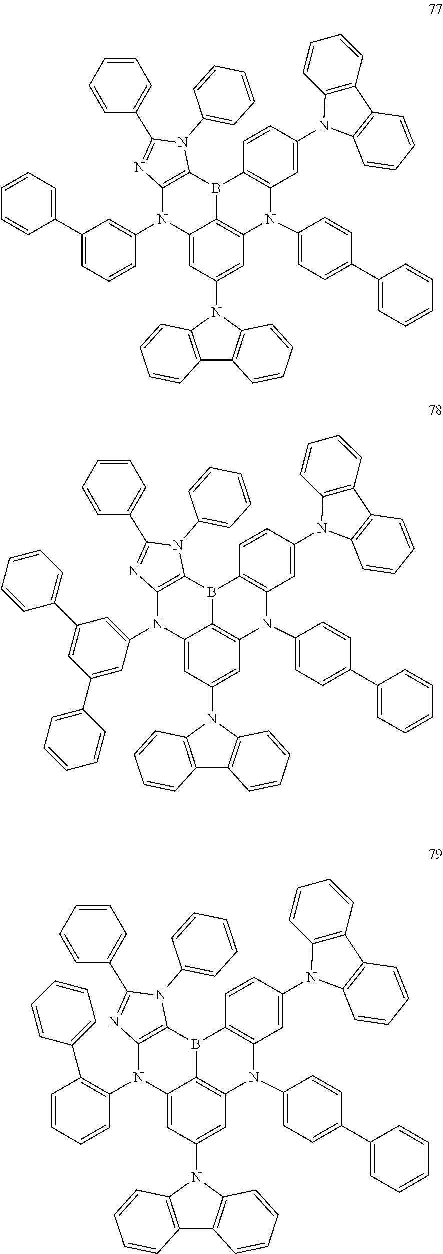 [SMILES: c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3nc(-c5ccccc5)n(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3nc(-c5ccccc5)n(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3nc(-c5ccccc5)n(-c5ccccc5)c34)cc2)cc1]

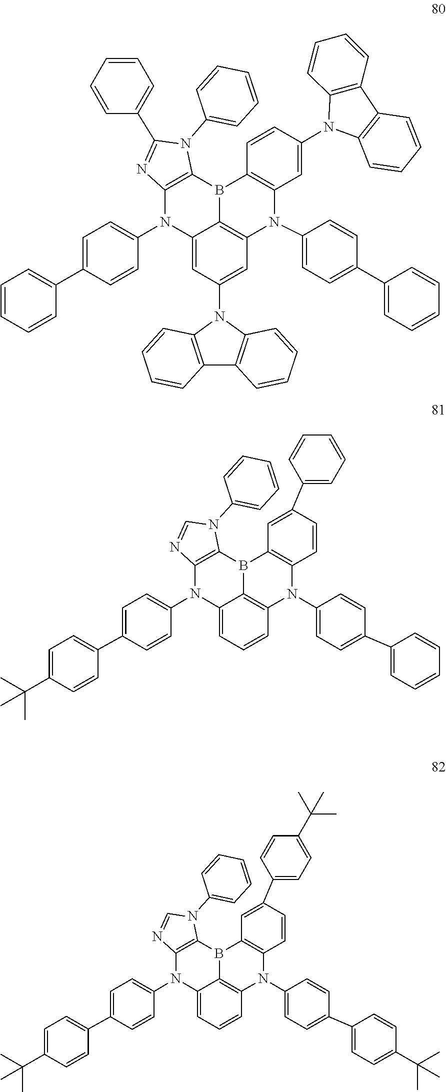 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; CC(C)(C)c1ccc(-c2ccc(N3c4cccc5c4B(c4cc(-c6ccccc6)ccc4N5c4ccc(-c5ccccc5)cc4)c4c3ncn4-c3ccccc3)cc2)cc1 ; CC(C)(C)c1ccc(-c2ccc(N3c4ccc(-c5ccc(C(C)(C)C)cc5)cc4B4c5c3cccc5N(c3ccc(-c5ccc(C(C)(C)C)cc5)cc3)c3ncn(-c5ccccc5)c34)cc2)cc1]

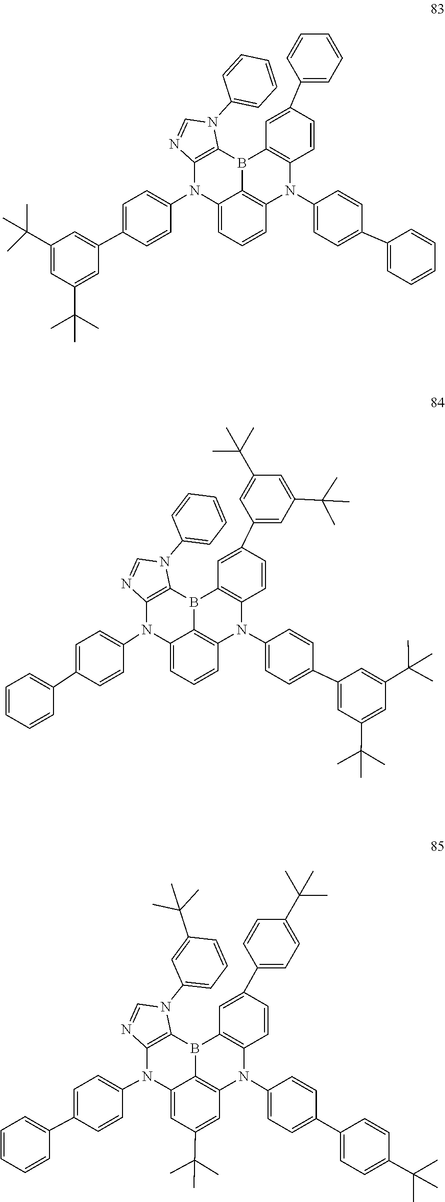 [SMILES: CC(C)(C)c1cc(-c2ccc(N3c4cccc5c4B(c4cc(-c6ccccc6)ccc4N5c4ccc(-c5ccccc5)cc4)c4c3ncn4-c3ccccc3)cc2)cc(C(C)(C)C)c1 ; CC(C)(C)c1cc(-c2ccc(N3c4ccc(-c5cc(C(C)(C)C)cc(C(C)(C)C)c5)cc4B4c5c3cccc5N(c3ccc(-c5ccccc5)cc3)c3ncn(-c5ccccc5)c34)cc2)cc(C(C)(C)C)c1 ; CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3ncn(-c5cccc(C(C)(C)C)c5)c34)cc2)cc1]

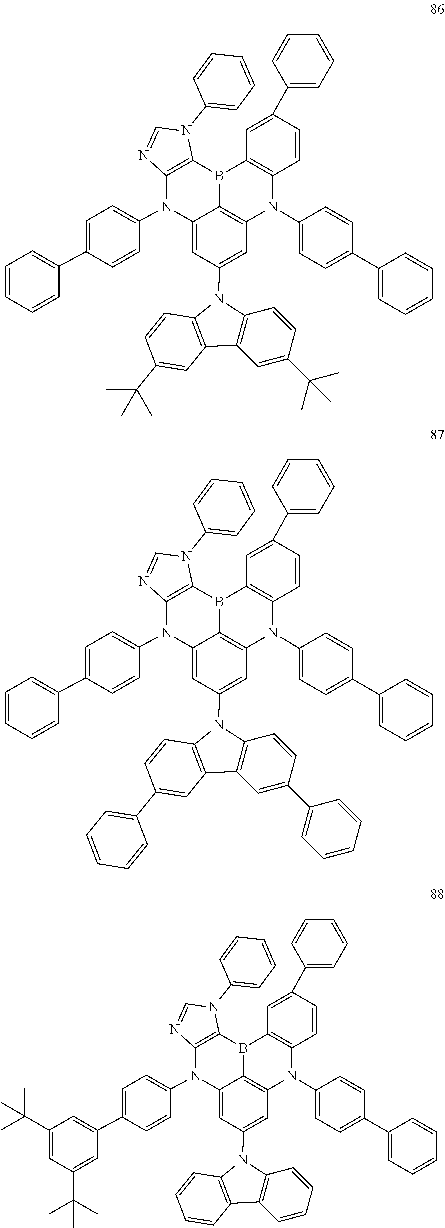 [SMILES: CC(C)(C)c1ccc2c(c1)c1cc(C(C)(C)C)ccc1n2-c1cc2c3c(c1)N(c1ccc(-c4ccccc4)cc1)c1ncn(-c4ccccc4)c1B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3ncn(-c5ccccc5)c34)cc2)cc1 ; CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4c3ncn4-c3ccccc3)cc2)cc(C(C)(C)C)c1]

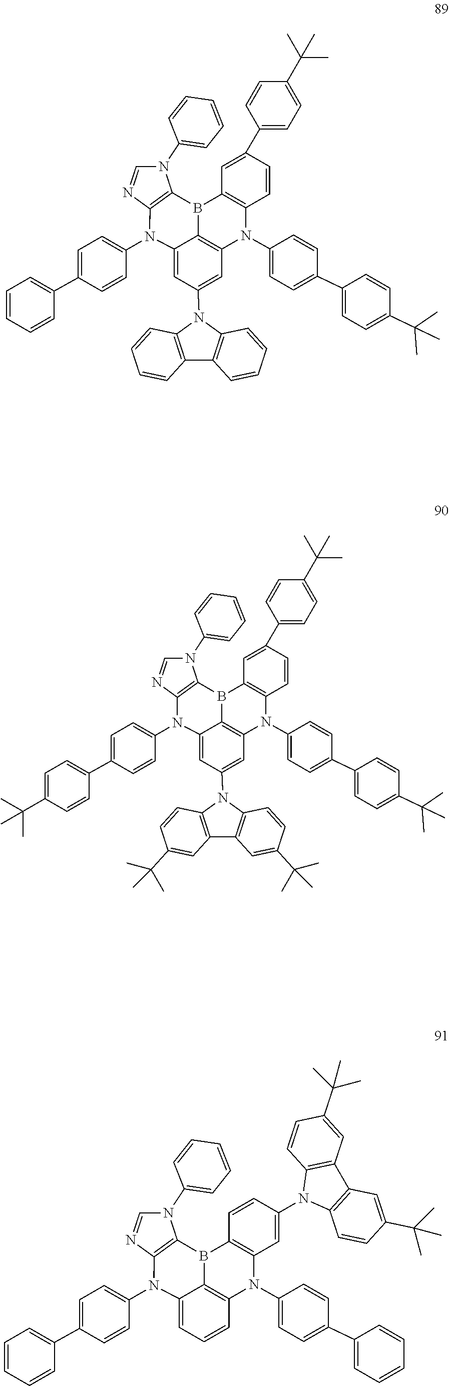

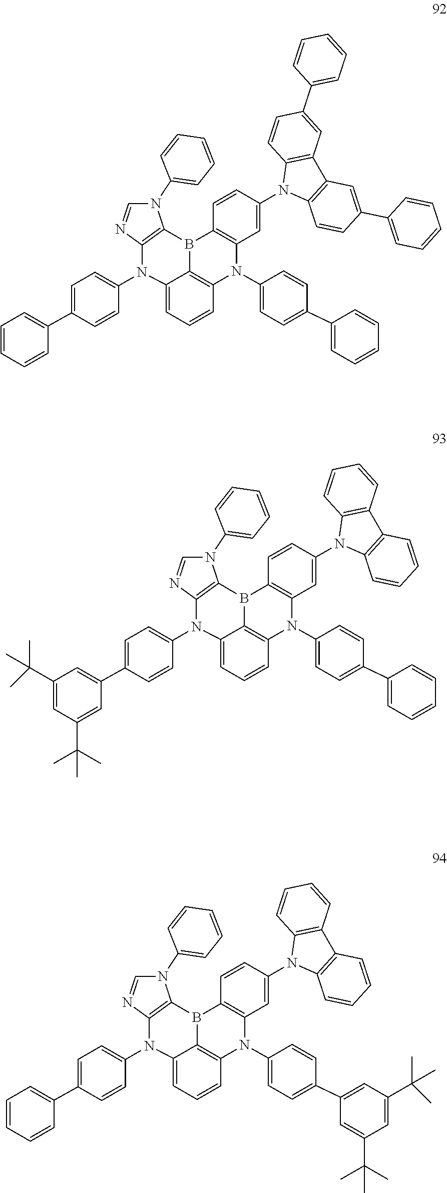 [SMILES: c1ccc(-c2ccc(N3c4cc(-n5c6ccc(-c7ccccc7)cc6c6cc(-c7ccccc7)ccc65)ccc4B4c5c3cccc5N(c3ccc(-c5ccccc5)cc3)c3ncn(-c5ccccc5)c34)cc2)cc1 ; CC(C)(C)c1cc(-c2ccc(N3c4cccc5c4B(c4ccc(-n6c7ccccc7c7ccccc76)cc4N5c4ccc(-c5ccccc5)cc4)c4c3ncn4-c3ccccc3)cc2)cc(C(C)(C)C)c1 ; CC(C)(C)c1cc(-c2ccc(N3c4cc(-n5c6ccccc6c6ccccc65)ccc4B4c5c3cccc5N(c3ccc(-c5ccccc5)cc3)c3ncn(-c5ccccc5)c34)cc2)cc(C(C)(C)C)c1]

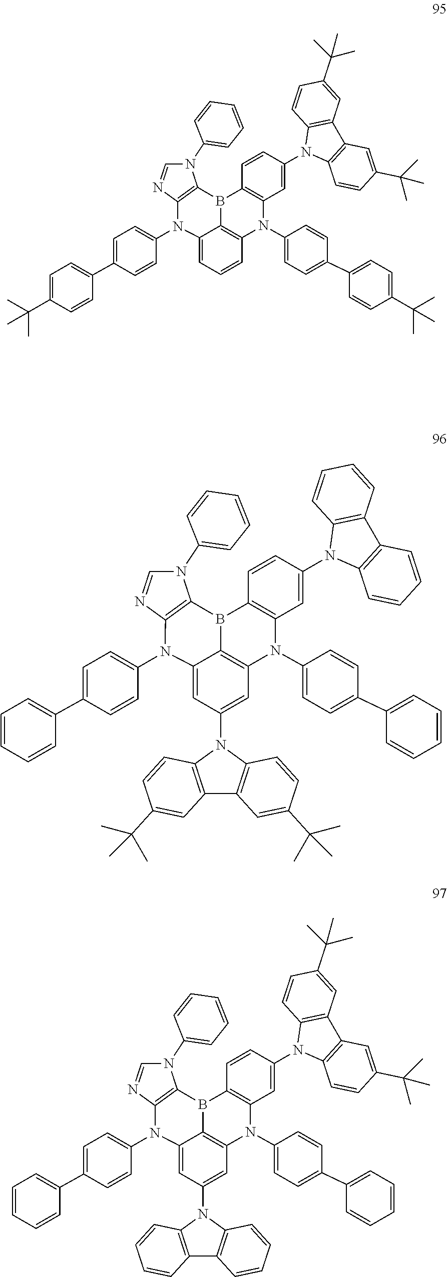 [SMILES: CC(C)(C)c1ccc(-c2ccc(N3c4cc(-n5c6ccc(C(C)(C)C)cc6c6cc(C(C)(C)C)ccc65)ccc4B4c5c3cccc5N(c3ccc(-c5ccc(C(C)(C)C)cc5)cc3)c3ncn(-c5ccccc5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1ncn(-c4ccccc4)c1B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(ncn1-c1ccccc1)N3c1ccc(-c2ccccc2)cc1]

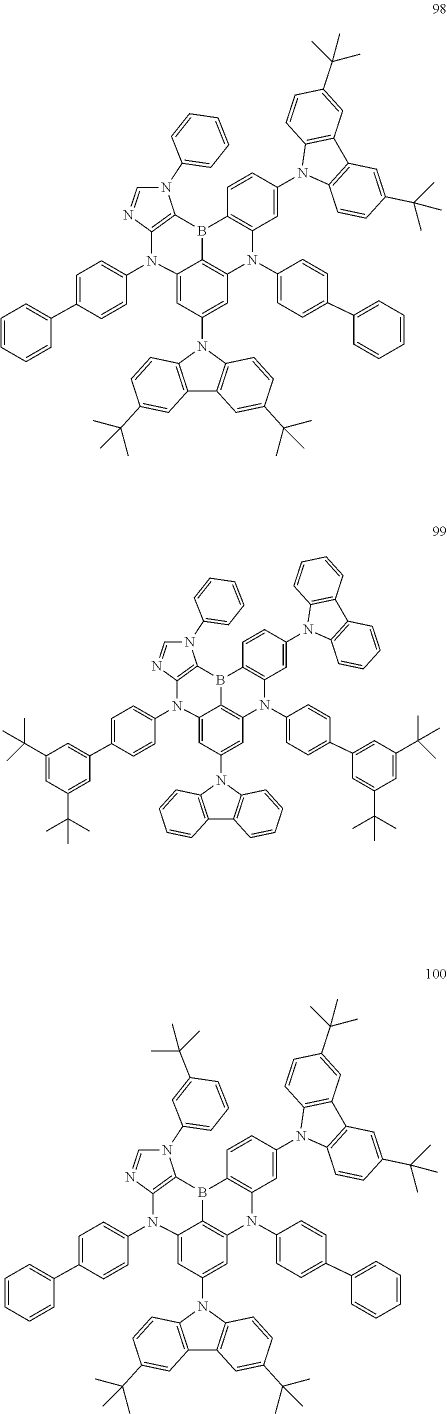

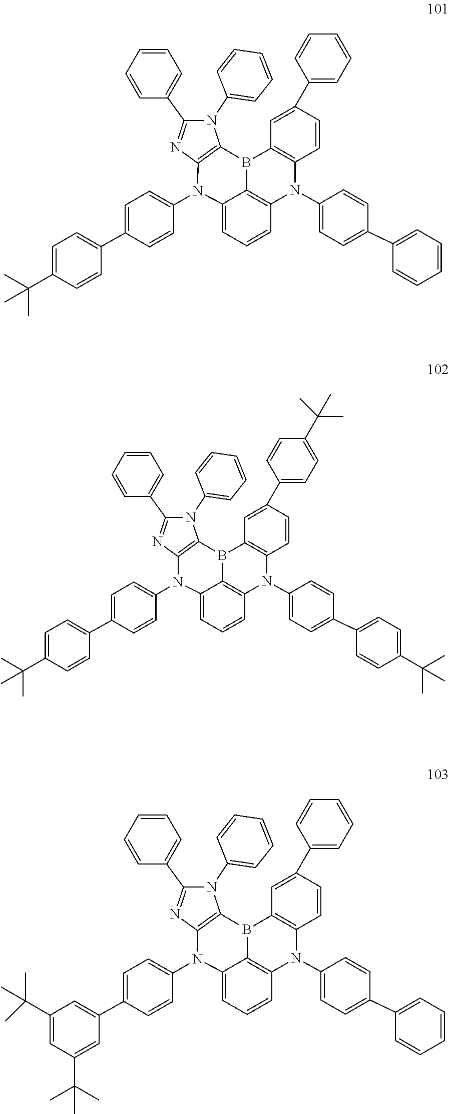

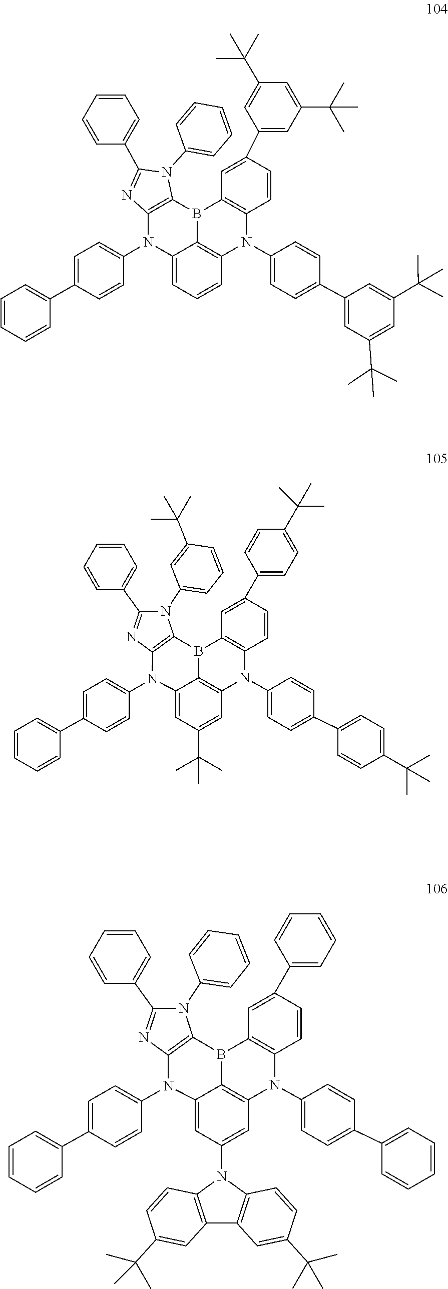 [SMILES: CC(C)(C)c1cc(-c2ccc(N3c4ccc(-c5cc(C(C)(C)C)cc(C(C)(C)C)c5)cc4B4c5c3cccc5N(c3ccc(-c5ccccc5)cc3)c3nc(-c5ccccc5)n(-c5ccccc5)c34)cc2)cc(C(C)(C)C)c1 ; CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3nc(-c5ccccc5)n(-c5cccc(C(C)(C)C)c5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1nc(-c4ccccc4)n(-c4ccccc4)c1B3c1cc(-c3ccccc3)ccc1N2c1ccc(-c2ccccc2)cc1]

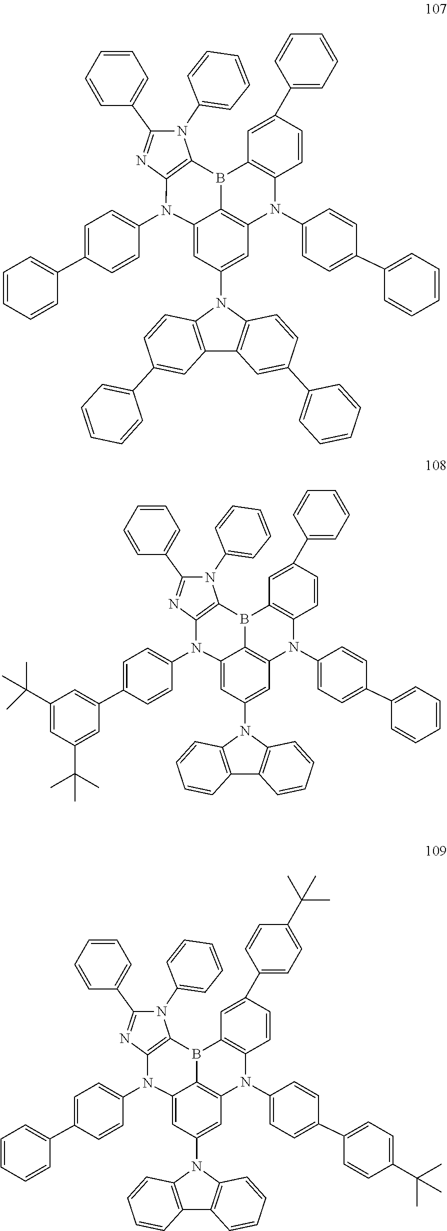 [SMILES: c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3nc(-c5ccccc5)n(-c5ccccc5)c34)cc2)cc1 ; CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4c3nc(-c3ccccc3)n4-c3ccccc3)cc2)cc(C(C)(C)C)c1 ; CC(C)(C)c1ccc(-c2ccc(N3c4ccc(-c5ccc(C(C)(C)C)cc5)cc4B4c5c3cc(-n3c6ccccc6c6ccccc63)cc5N(c3ccc(-c5ccccc5)cc3)c3nc(-c5ccccc5)n(-c5ccccc5)c34)cc2)cc1]

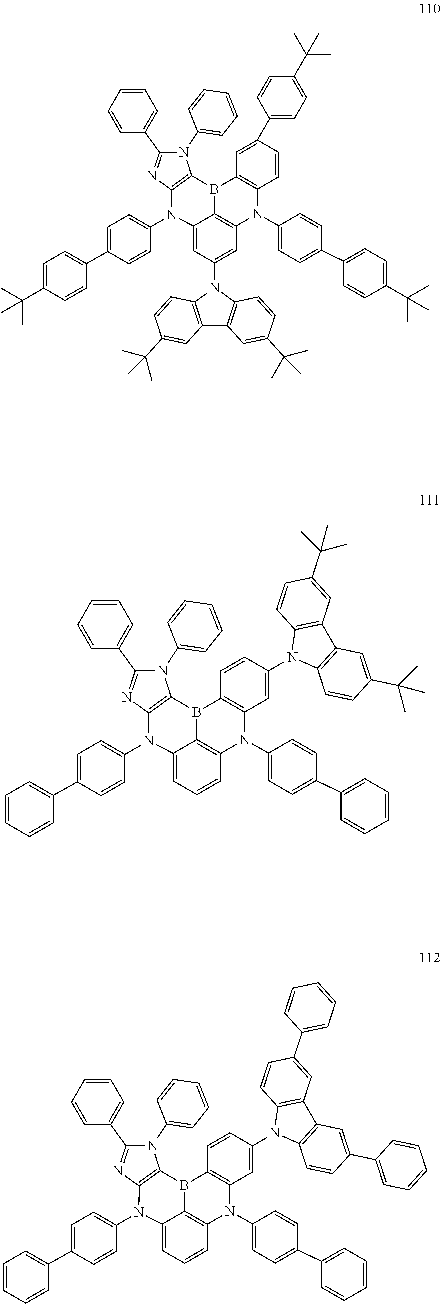 [SMILES: CC(C)(C)c1ccc(-c2ccc(N3c4ccc(-c5ccc(C(C)(C)C)cc5)cc4B4c5c3cc(-n3c6ccc(C(C)(C)C)cc6c6cc(C(C)(C)C)ccc63)cc5N(c3ccc(-c5ccc(C(C)(C)C)cc5)cc3)c3nc(-c5ccccc5)n(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(nc(-c2ccccc2)n1-c1ccccc1)N3c1ccc(-c2ccccc2)cc1 ; c1ccc(-c2ccc(N3c4cc(-n5c6ccc(-c7ccccc7)cc6c6cc(-c7ccccc7)ccc65)ccc4B4c5c3cccc5N(c3ccc(-c5ccccc5)cc3)c3nc(-c5ccccc5)n(-c5ccccc5)c34)cc2)cc1]

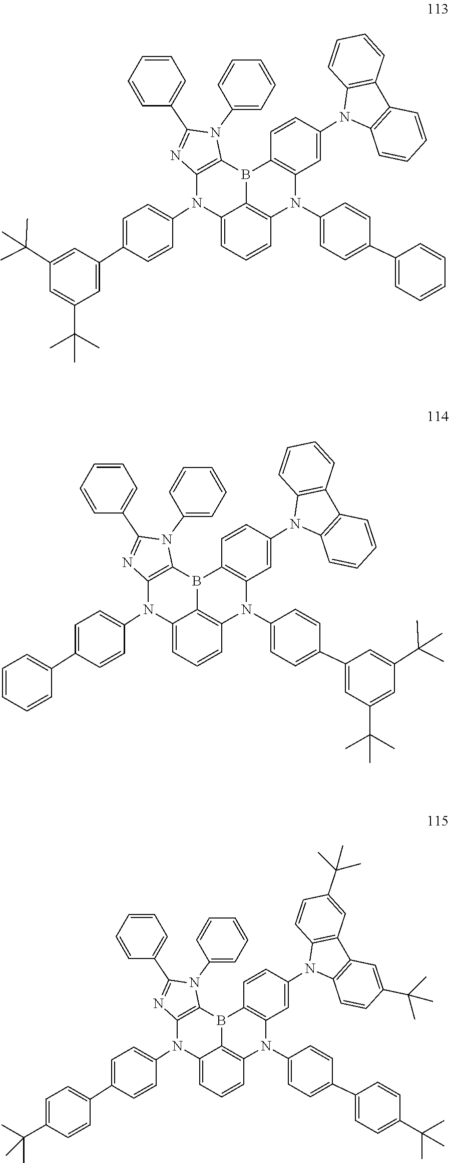

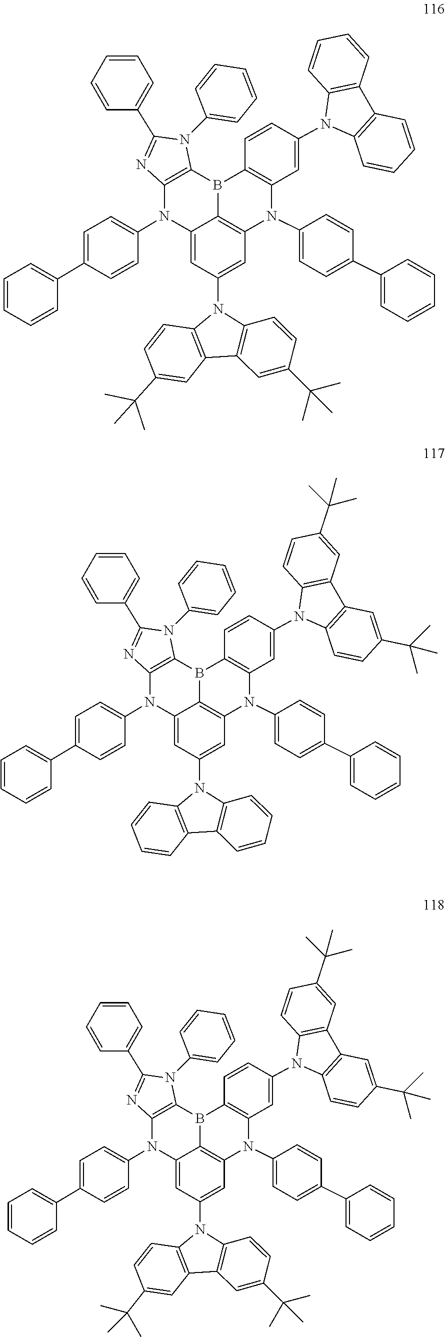 [SMILES: CC(C)(C)c1ccc2c(c1)c1cc(C(C)(C)C)ccc1n2-c1cc2c3c(c1)N(c1ccc(-c4ccccc4)cc1)c1nc(-c4ccccc4)n(-c4ccccc4)c1B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(nc(-c2ccccc2)n1-c1ccccc1)N3c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccc(C(C)(C)C)cc4c4cc(C(C)(C)C)ccc43)cc3c1B2c1c(nc(-c2ccccc2)n1-c1ccccc1)N3c1ccc(-c2ccccc2)cc1]

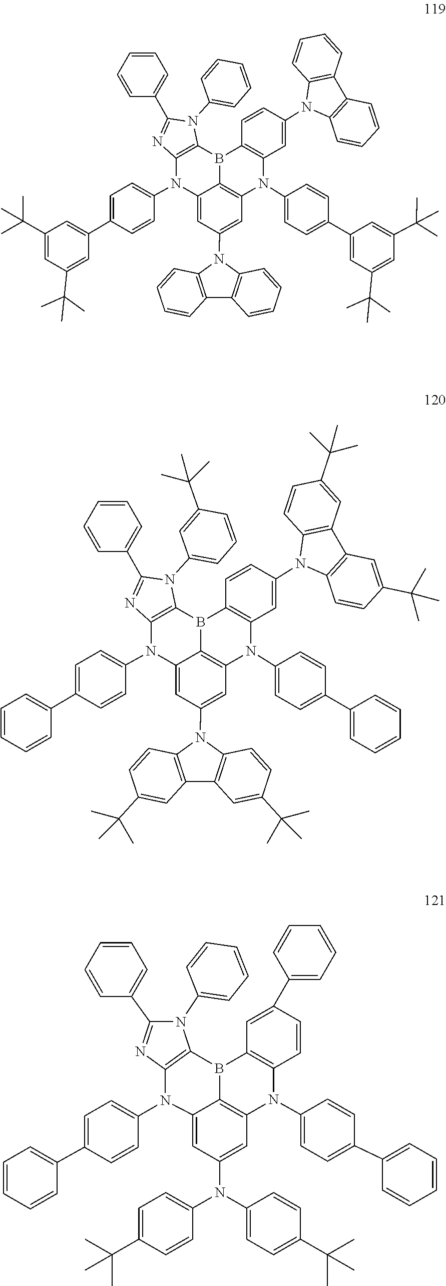

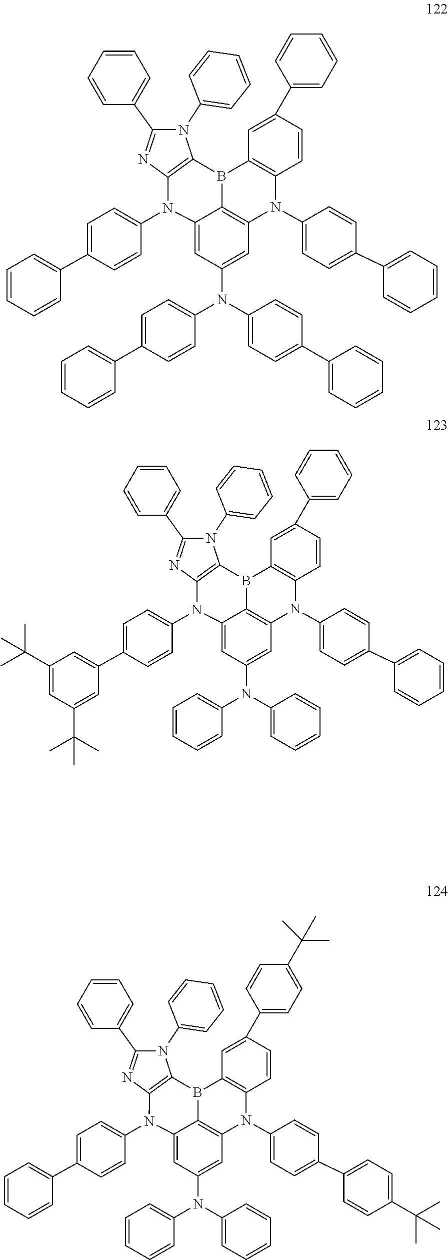 [SMILES: c1ccc(-c2ccc(N(c3ccc(-c4ccccc4)cc3)c3cc4c5c(c3)N(c3ccc(-c6ccccc6)cc3)c3nc(-c6ccccc6)n(-c6ccccc6)c3B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c3nc(-c3ccccc3)n4-c3ccccc3)cc2)cc(C(C)(C)C)c1 ; CC(C)(C)c1ccc(-c2ccc(N3c4ccc(-c5ccc(C(C)(C)C)cc5)cc4B4c5c3cc(N(c3ccccc3)c3ccccc3)cc5N(c3ccc(-c5ccccc5)cc3)c3nc(-c5ccccc5)n(-c5ccccc5)c34)cc2)cc1]

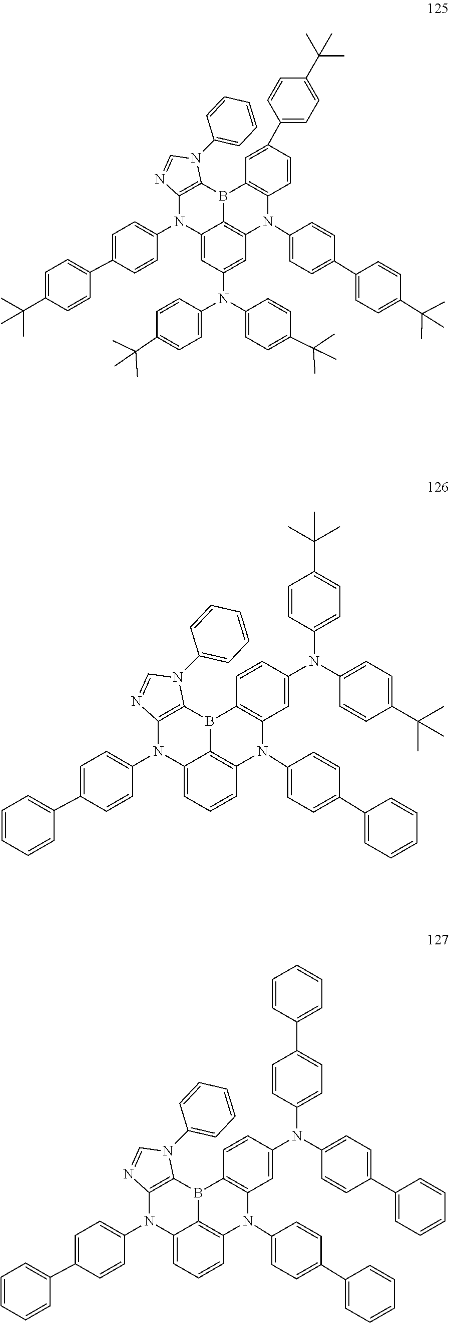 [SMILES: CC(C)(C)c1ccc(-c2ccc(N3c4ccc(-c5ccc(C(C)(C)C)cc5)cc4B4c5c3cc(N(c3ccc(C(C)(C)C)cc3)c3ccc(C(C)(C)C)cc3)cc5N(c3ccc(-c5ccc(C(C)(C)C)cc5)cc3)c3ncn(-c5ccccc5)c34)cc2)cc1 ; CC(C)(C)c1ccc(N(c2ccc(C(C)(C)C)cc2)c2ccc3c(c2)N(c2ccc(-c4ccccc4)cc2)c2cccc4c2B3c2c(ncn2-c2ccccc2)N4c2ccc(-c3ccccc3)cc2)cc1 ; c1ccc(-c2ccc(N(c3ccc(-c4ccccc4)cc3)c3ccc4c(c3)N(c3ccc(-c5ccccc5)cc3)c3cccc5c3B4c3c(ncn3-c3ccccc3)N5c3ccc(-c4ccccc4)cc3)cc2)cc1]

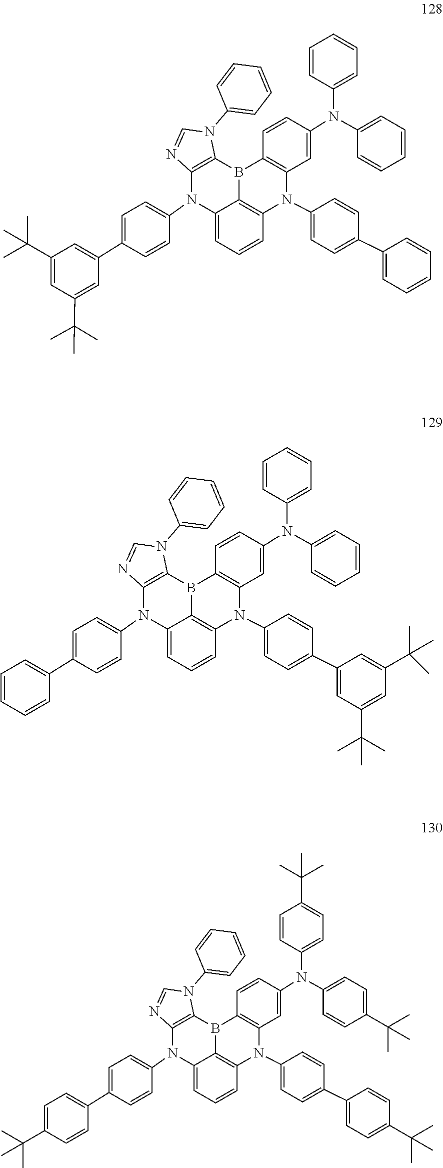

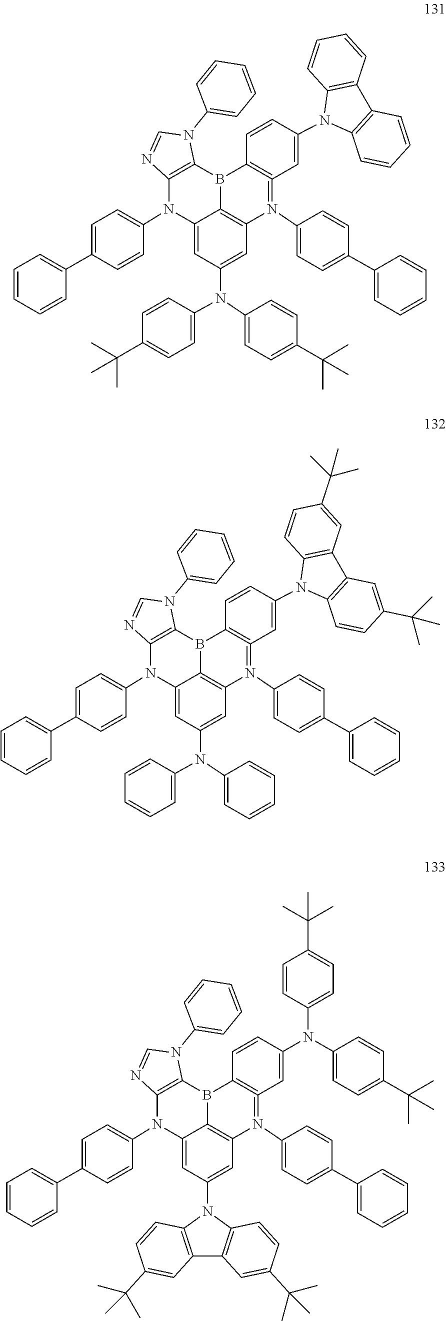

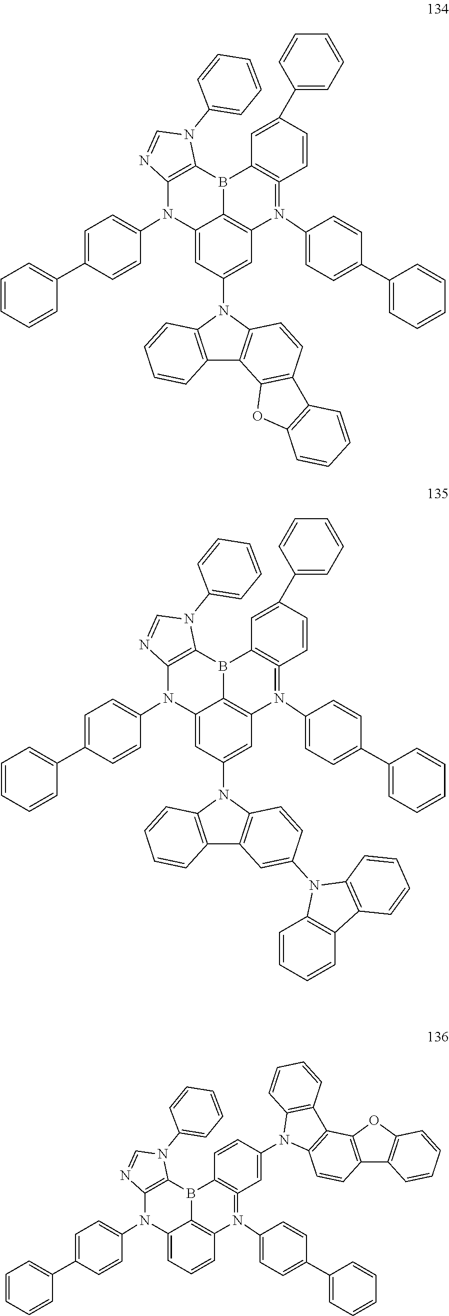

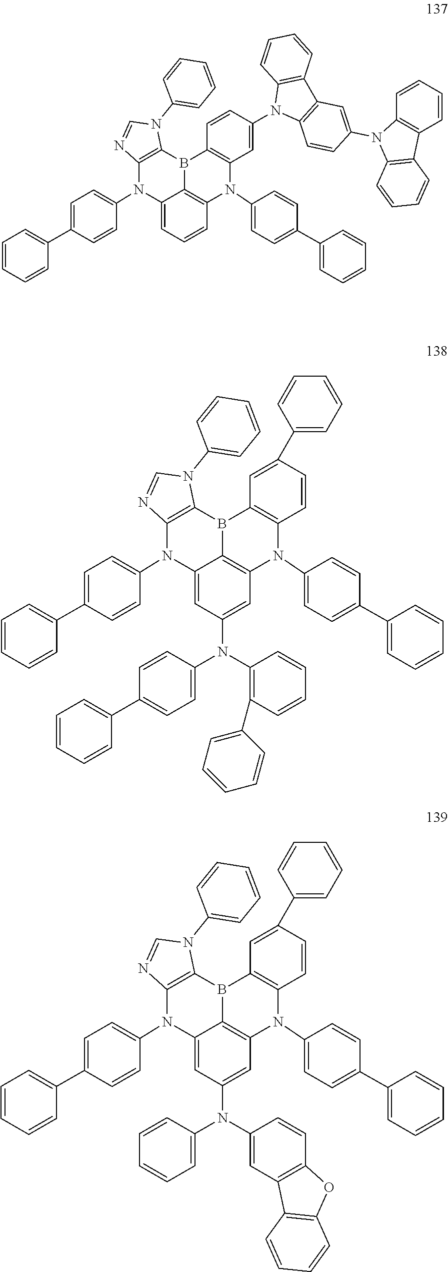 [SMILES: c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3ncn(-c5ccccc5)c34)cc2)cc1 ; c1ccc(-c2ccc(N(c3cc4c5c(c3)N(c3ccc(-c6ccccc6)cc3)c3ncn(-c6ccccc6)c3B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3ncn(-c5ccccc5)c34)cc2)cc1]

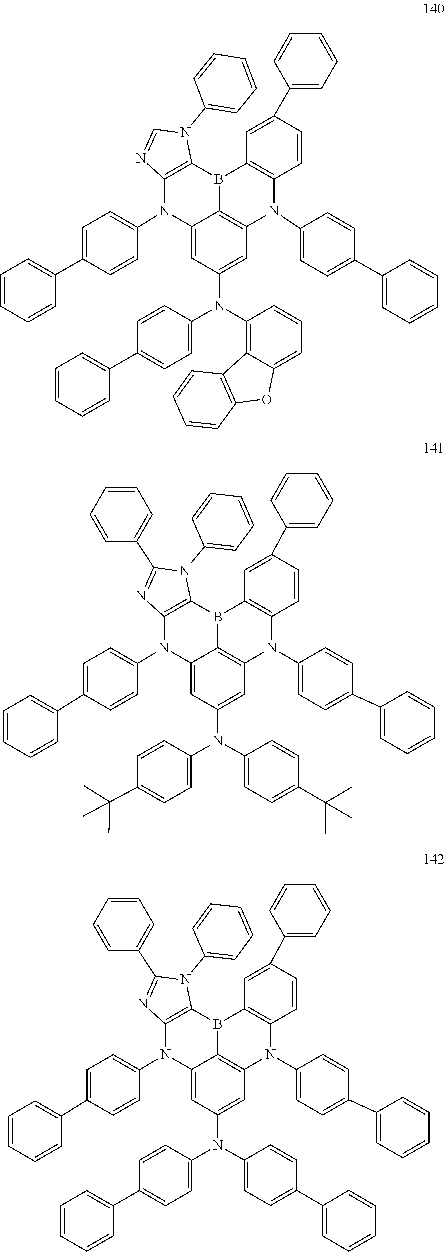 [SMILES: c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3ncn(-c5ccccc5)c34)cc2)cc1 ; CC(C)(C)c1ccc(N(c2ccc(C(C)(C)C)cc2)c2cc3c4c(c2)N(c2ccc(-c5ccccc5)cc2)c2nc(-c5ccccc5)n(-c5ccccc5)c2B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3nc(-c6ccccc6)n(-c6ccccc6)c3B5c3cc(-c5ccccc5)ccc3N4c3ccc(-c4ccccc4)cc3)cc2)cc1]

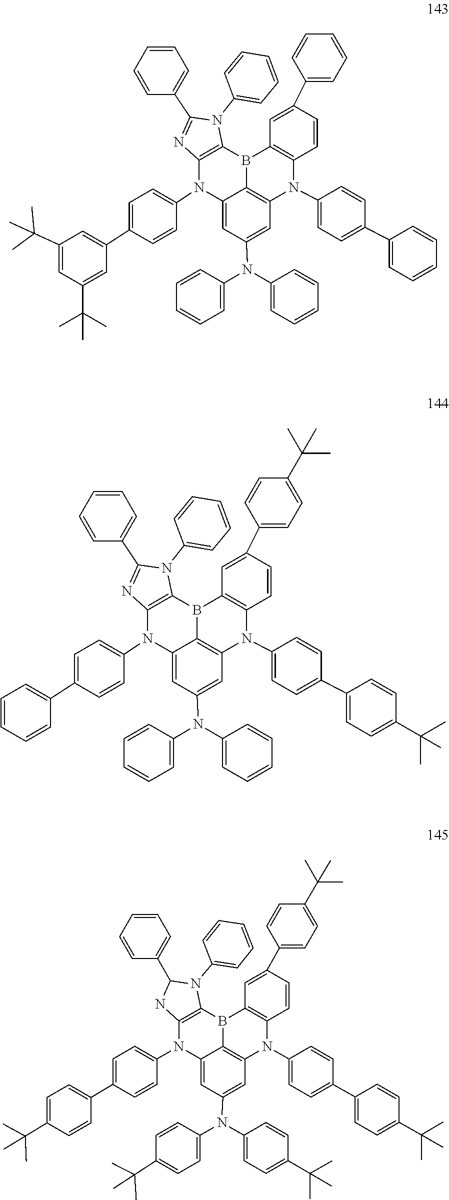

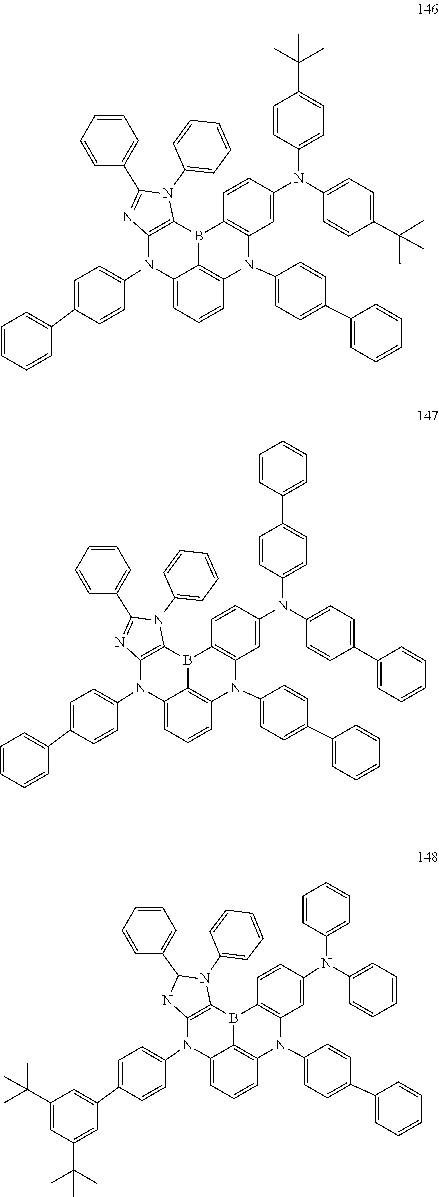

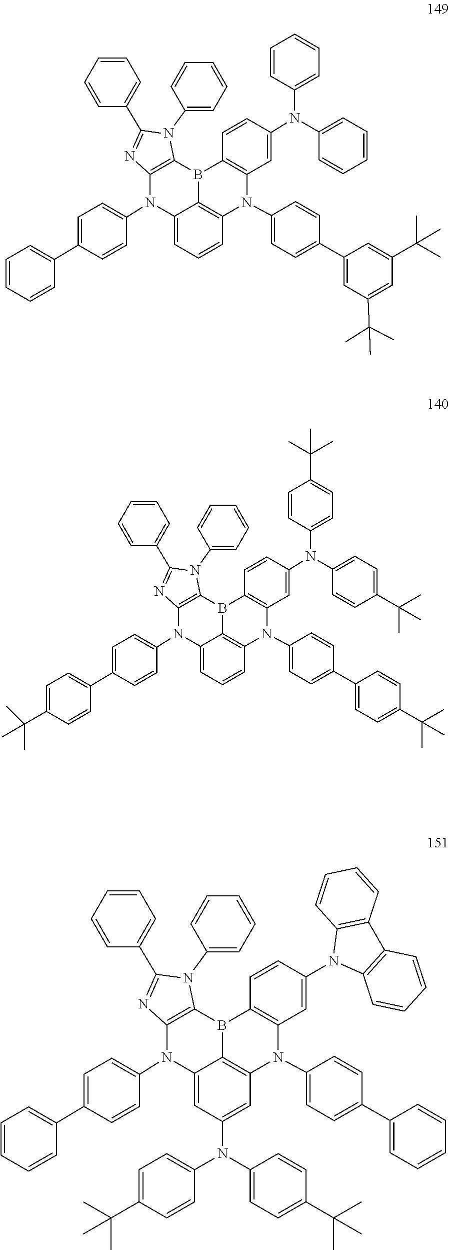

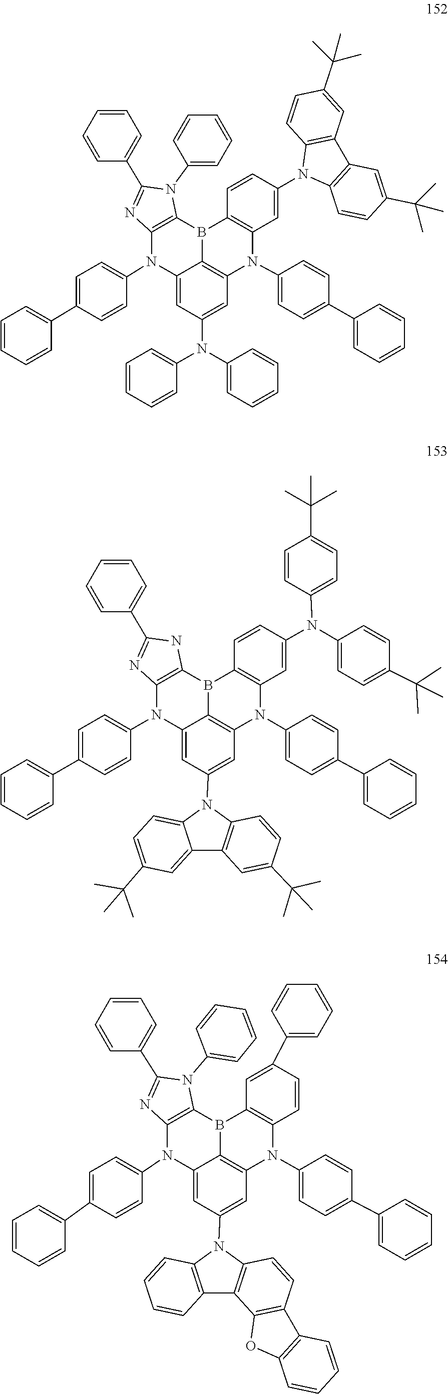 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(nc(-c2ccccc2)n1-c1ccccc1)N3c1ccc(-c2ccccc2)cc1 ; CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2[nH]c(-c3ccccc3)nc2N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3nc(-c5ccccc5)n(-c5ccccc5)c34)cc2)cc1]

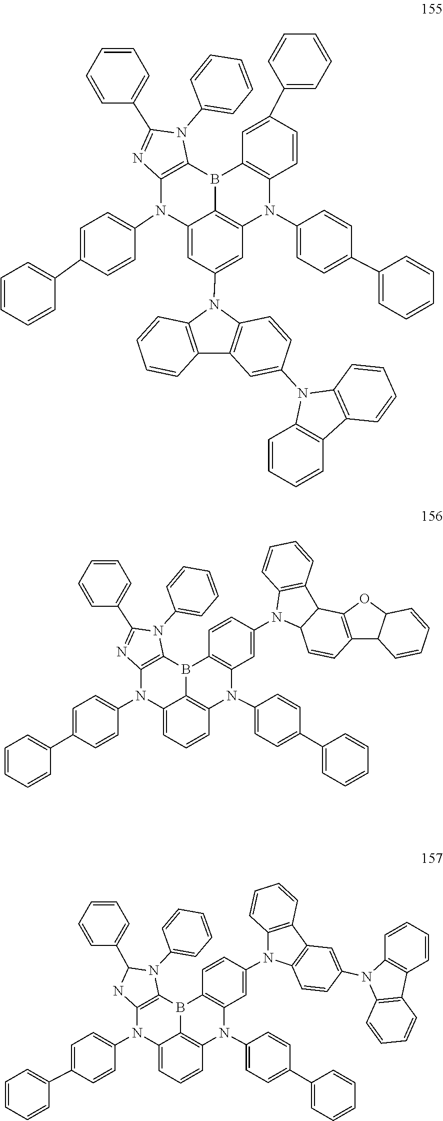

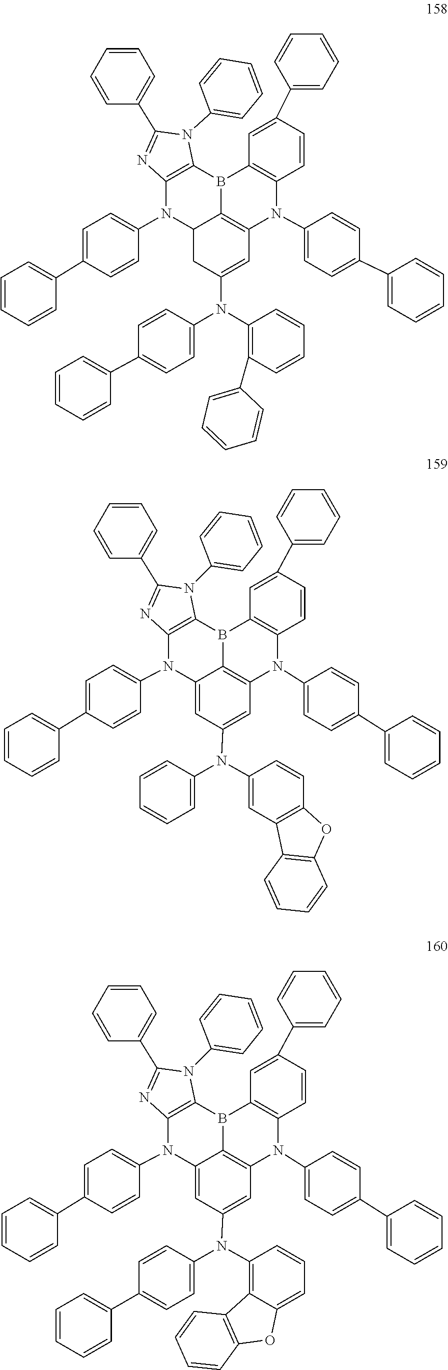 [SMILES: C1=C(N(c2ccc(-c3ccccc3)cc2)c2ccccc2-c2ccccc2)CC2C3=C1N(c1ccc(-c4ccccc4)cc1)c1ccc(-c4ccccc4)cc1B3c1c(nc(-c3ccccc3)n1-c1ccccc1)N2c1ccc(-c2ccccc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3nc(-c5ccccc5)n(-c5ccccc5)c34)cc2)cc1 ; c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3nc(-c5ccccc5)n(-c5ccccc5)c34)cc2)cc1]

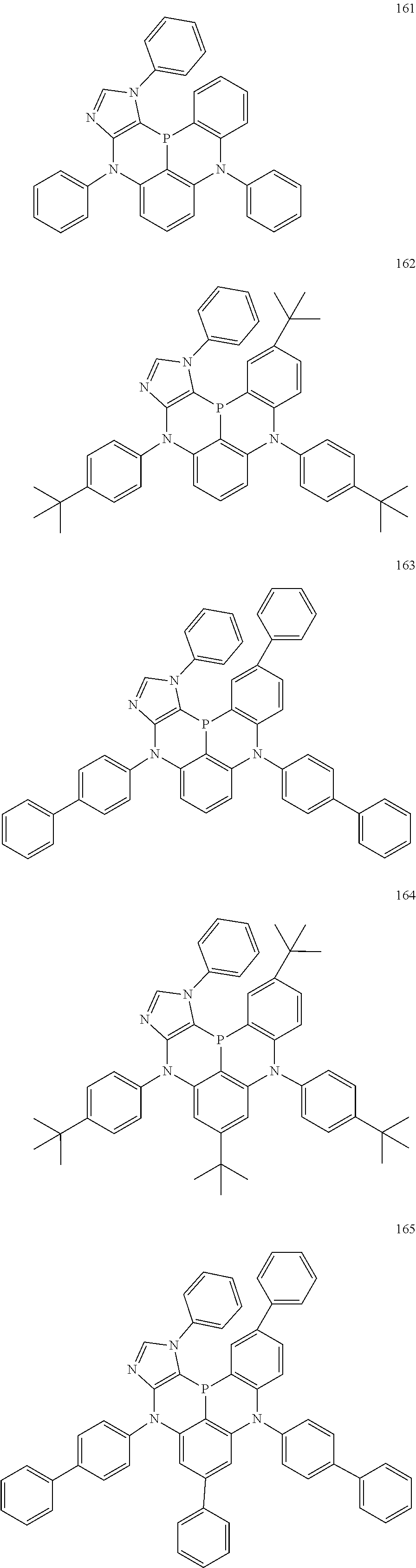

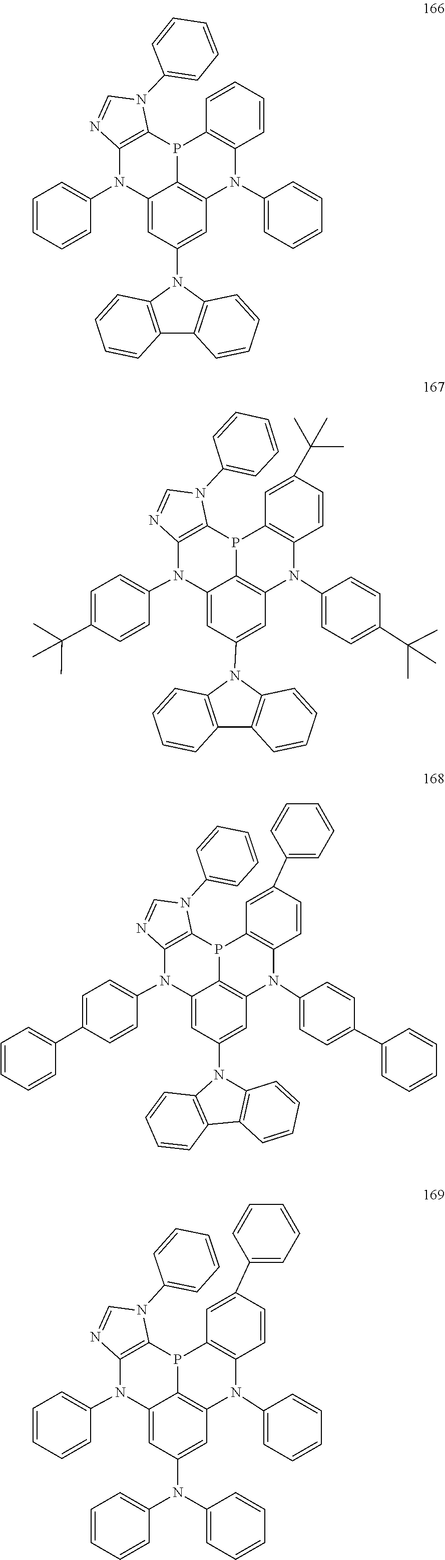 [SMILES: c1ccc(N2c3ccccc3P3c4c2cc(-n2c5ccccc5c5ccccc52)cc4N(c2ccccc2)c2ncn(-c4ccccc4)c23)cc1 ; CC(C)(C)c1ccc(N2c3ccc(C(C)(C)C)cc3P3c4c2cc(-n2c5ccccc5c5ccccc52)cc4N(c2ccc(C(C)(C)C)cc2)c2ncn(-c4ccccc4)c23)cc1 ; c1ccc(-c2ccc(N3c4ccc(-c5ccccc5)cc4P4c5c3cc(-n3c6ccccc6c6ccccc63)cc5N(c3ccc(-c5ccccc5)cc3)c3ncn(-c5ccccc5)c34)cc2)cc1 ; c1ccc(-c2ccc3c(c2)P2c4c(cc(N(c5ccccc5)c5ccccc5)cc4N(c4ccccc4)c4ncn(-c5ccccc5)c42)N3c2ccccc2)cc1]

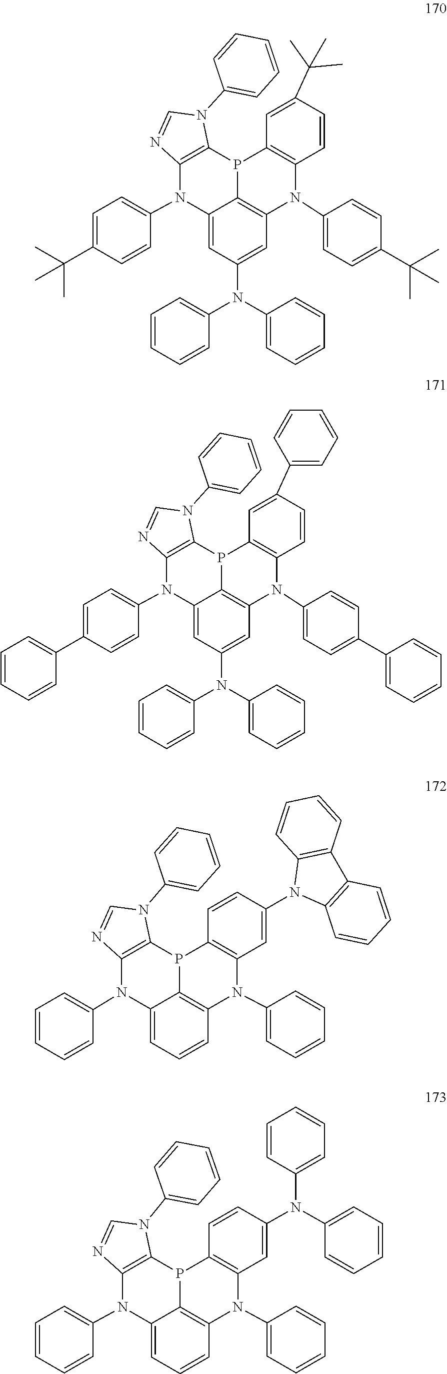

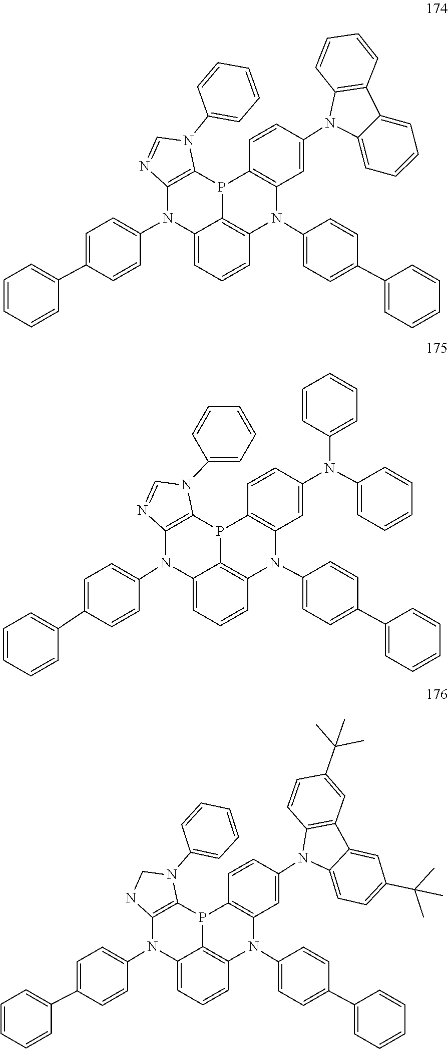

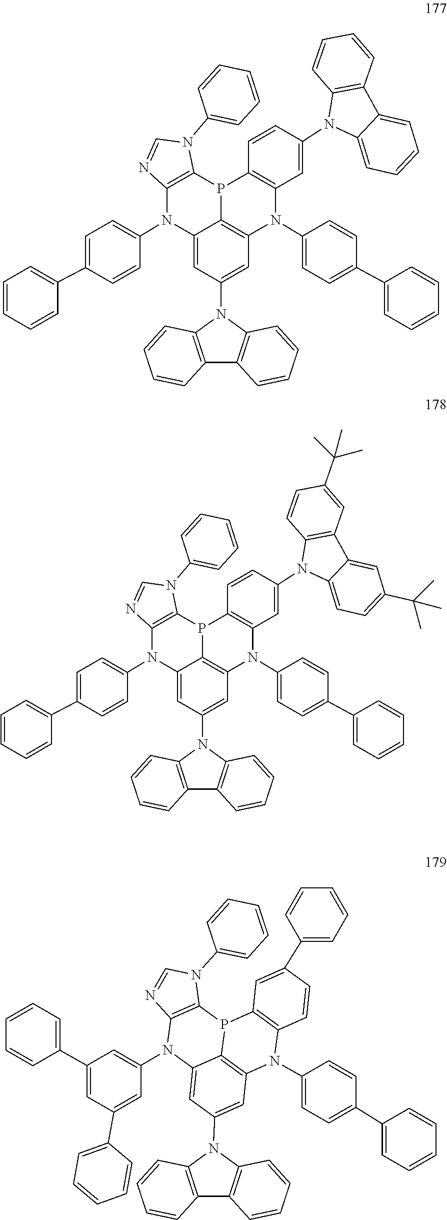 [SMILES: c1ccc(-c2ccc(N3c4cc(-n5c6ccccc6c6ccccc65)ccc4P4c5c3cc(-n3c6ccccc6c6ccccc63)cc5N(c3ccc(-c5ccccc5)cc3)c3ncn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1c(ncn1-c1ccccc1)N3c1ccc(-c2ccccc2)cc1 ; c1ccc(-c2ccc(N3c4ccc(-c5ccccc5)cc4P4c5c3cc(-n3c6ccccc6c6ccccc63)cc5N(c3cc(-c5ccccc5)cc(-c5ccccc5)c3)c3ncn(-c5ccccc5)c34)cc2)cc1]

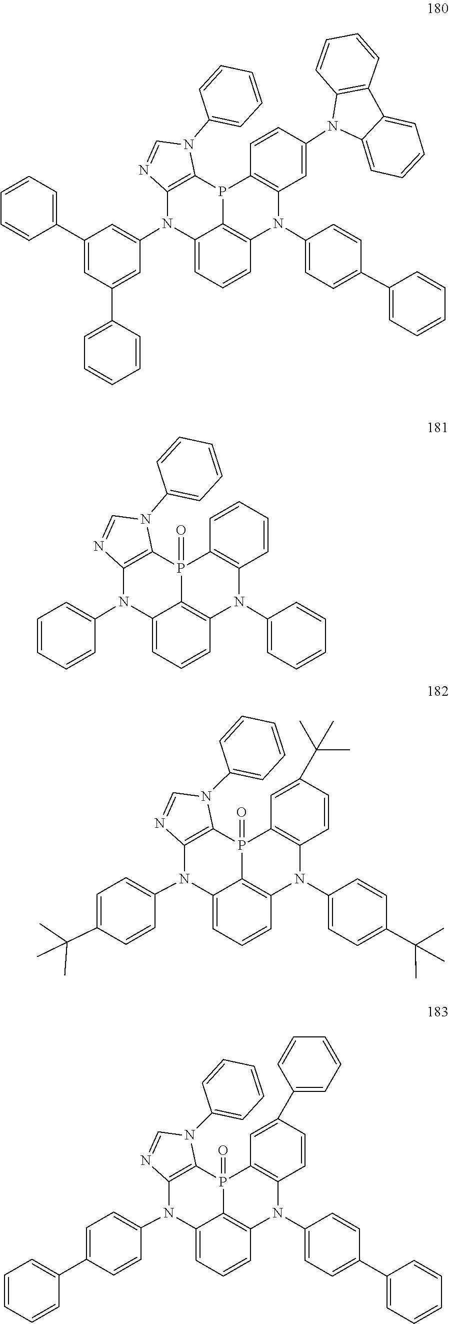

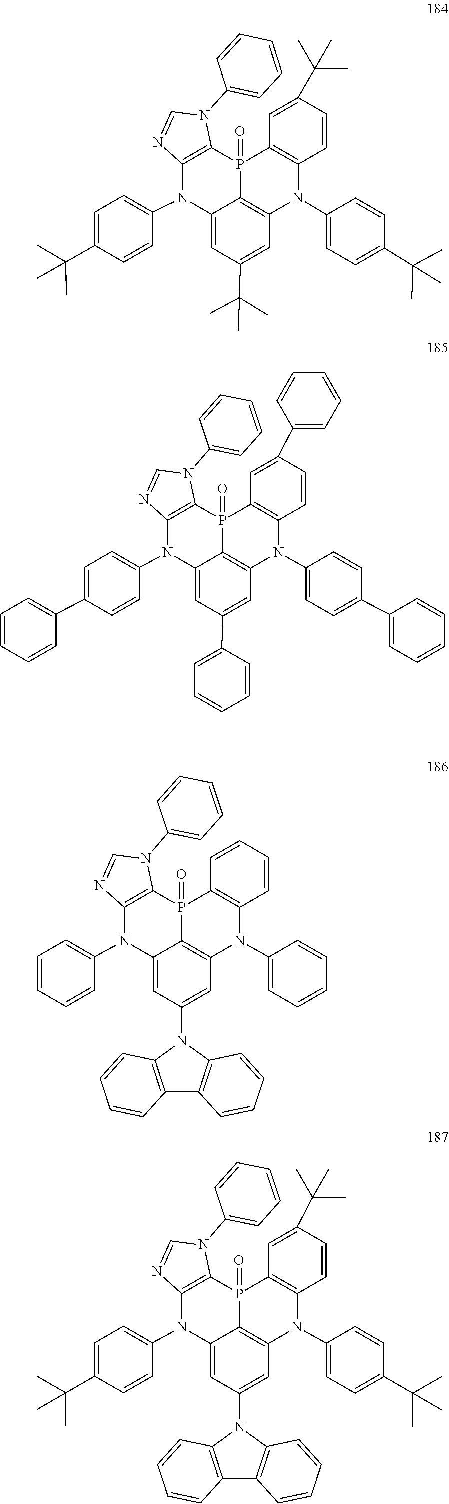

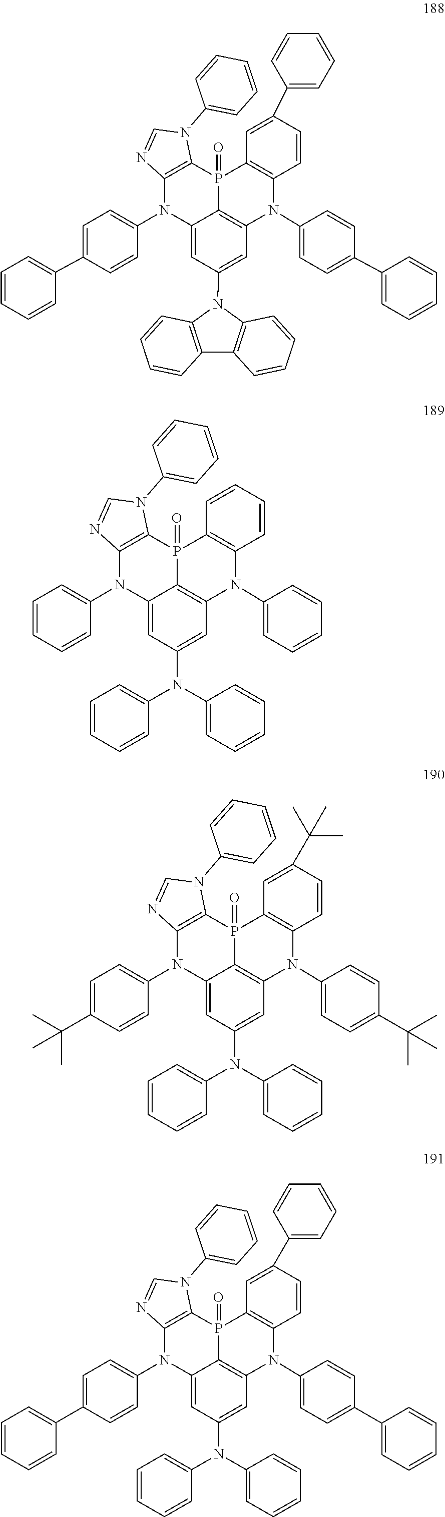 [SMILES: O=P12c3cc(-c4ccccc4)ccc3N(c3ccc(-c4ccccc4)cc3)c3cc(-n4c5ccccc5c5ccccc54)cc(c31)N(c1ccc(-c3ccccc3)cc1)c1ncn(-c3ccccc3)c12 ; O=P12c3ccccc3N(c3ccccc3)c3cc(N(c4ccccc4)c4ccccc4)cc(c31)N(c1ccccc1)c1ncn(-c3ccccc3)c12 ; CC(C)(C)c1ccc(N2c3ccc(C(C)(C)C)cc3P3(=O)c4c2cc(N(c2ccccc2)c2ccccc2)cc4N(c2ccc(C(C)(C)C)cc2)c2ncn(-c4ccccc4)c23)cc1 ; O=P12c3cc(-c4ccccc4)ccc3N(c3ccc(-c4ccccc4)cc3)c3cc(N(c4ccccc4)c4ccccc4)cc(c31)N(c1ccc(-c3ccccc3)cc1)c1ncn(-c3ccccc3)c12]

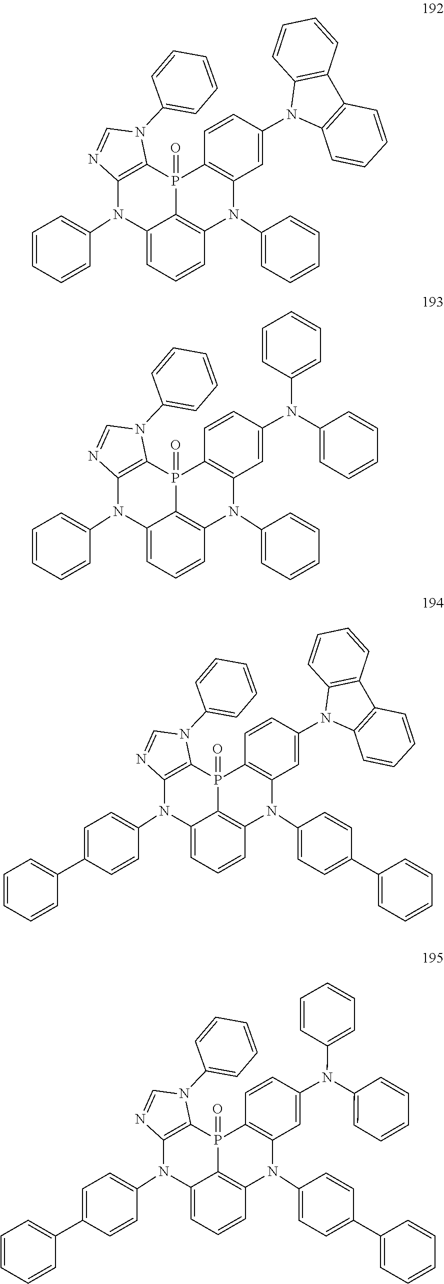

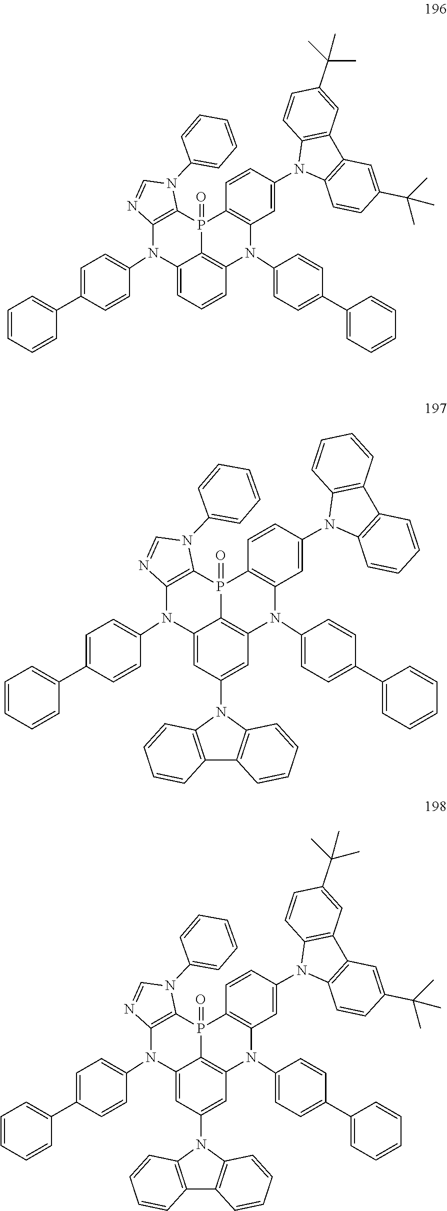 [SMILES: CC(C)(C)c1ccc2c(c1)c1cc(C(C)(C)C)ccc1n2-c1ccc2c(c1)N(c1ccc(-c3ccccc3)cc1)c1cccc3c1P2(=O)c1c(ncn1-c1ccccc1)N3c1ccc(-c2ccccc2)cc1 ; O=P12c3ccc(-n4c5ccccc5c5ccccc54)cc3N(c3ccc(-c4ccccc4)cc3)c3cc(-n4c5ccccc5c5ccccc54)cc(c31)N(c1ccc(-c3ccccc3)cc1)c1ncn(-c3ccccc3)c12 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2(=O)c1c(ncn1-c1ccccc1)N3c1ccc(-c2ccccc2)cc1]

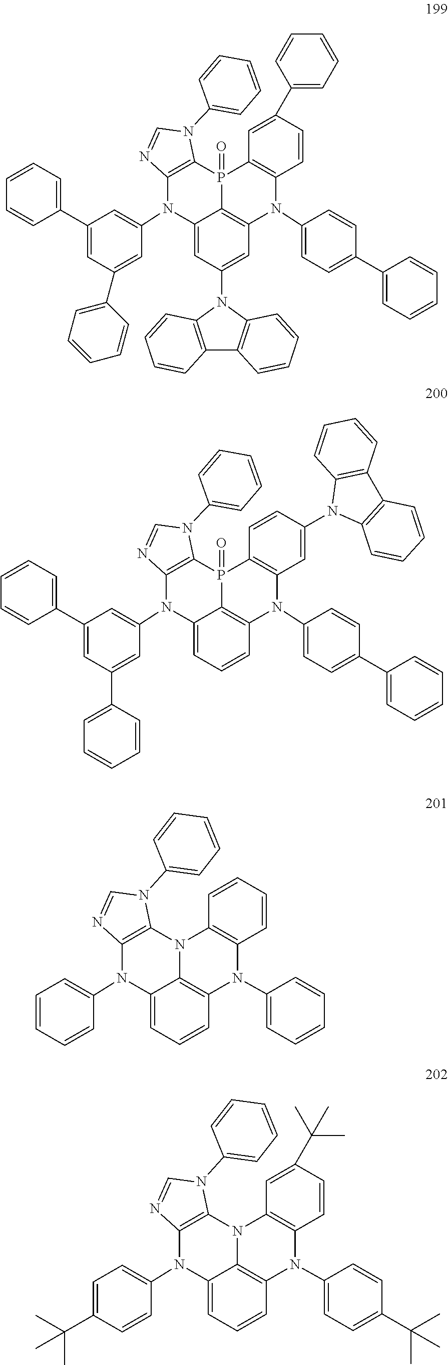 [SMILES: O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1ncn(-c3ccccc3)c12 ; O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1ncn(-c3ccccc3)c12 ; c1ccc(N2c3ccccc3N3c4c2cccc4N(c2ccccc2)c2ncn(-c4ccccc4)c23)cc1 ; CC(C)(C)c1ccc(N2c3ccc(C(C)(C)C)cc3N3c4c2cccc4N(c2ccc(C(C)(C)C)cc2)c2ncn(-c4ccccc4)c23)cc1]

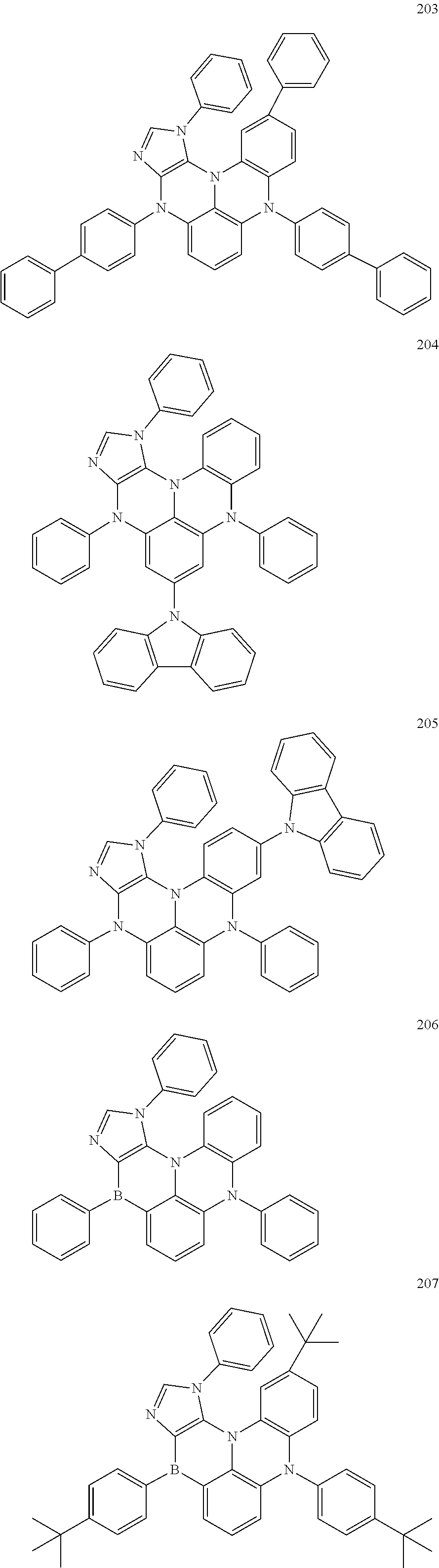

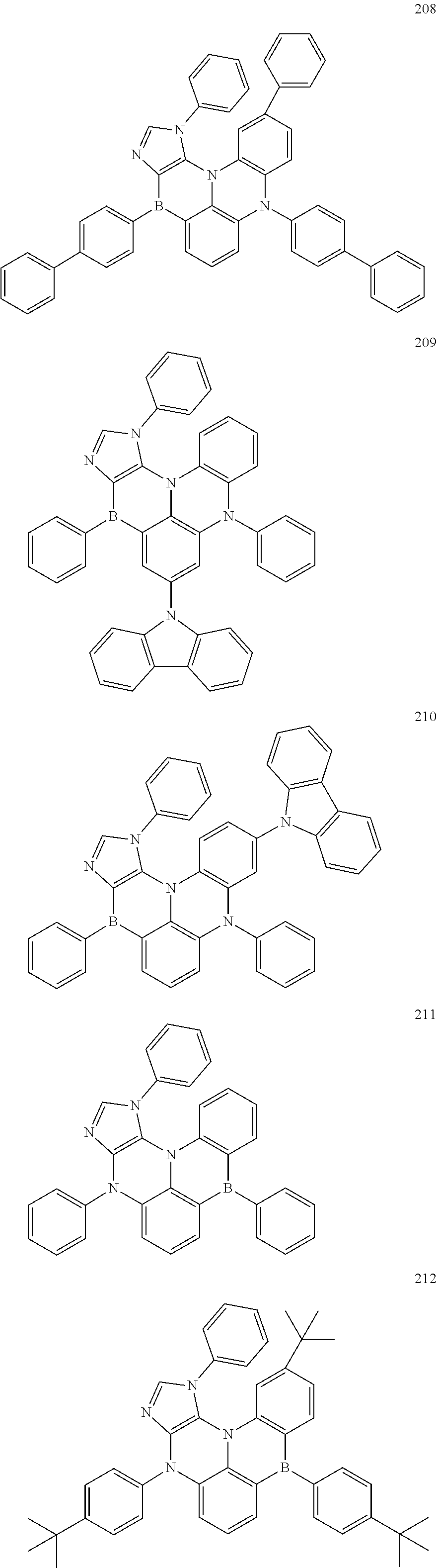

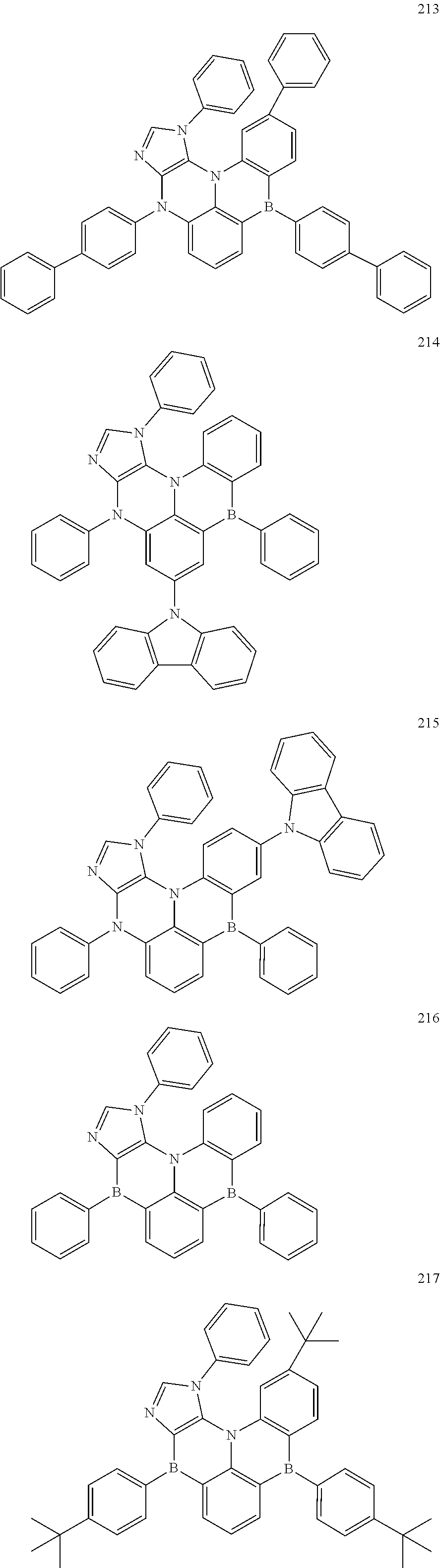

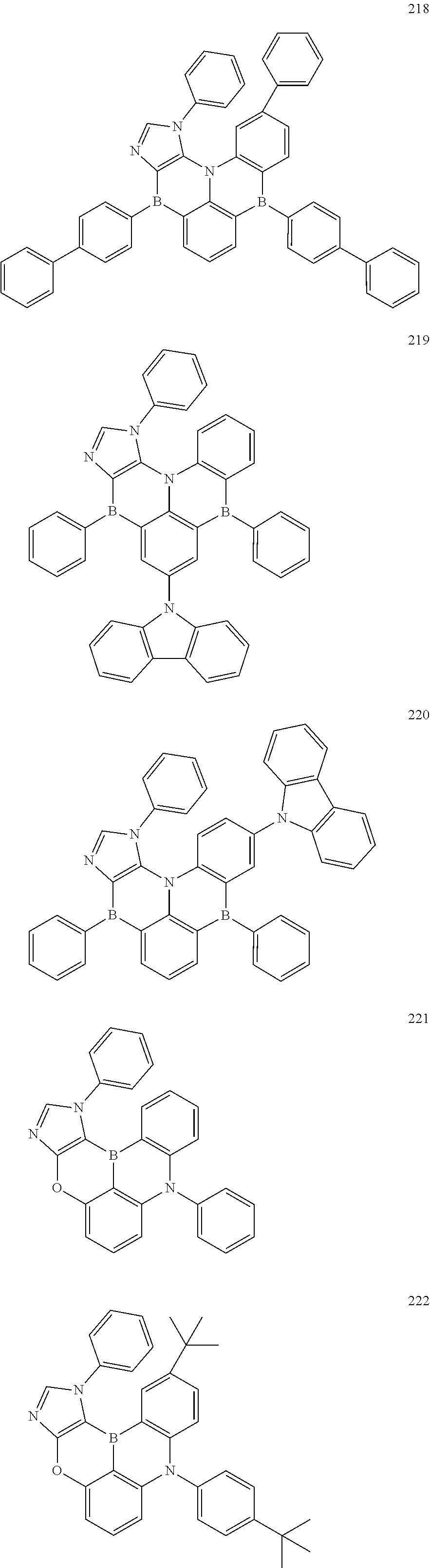 [SMILES: c1ccc(-c2ccc(B3c4ccc(-c5ccccc5)cc4N4c5c3cccc5B(c3ccc(-c5ccccc5)cc3)c3ncn(-c5ccccc5)c34)cc2)cc1 ; c1ccc(B2c3ccccc3N3c4c2cc(-n2c5ccccc5c5ccccc52)cc4B(c2ccccc2)c2ncn(-c4ccccc4)c23)cc1 ; c1ccc(B2c3cc(-n4c5ccccc5c5ccccc54)ccc3N3c4c2cccc4B(c2ccccc2)c2ncn(-c4ccccc4)c23)cc1 ; c1ccc(N2c3ccccc3B3c4c(cccc42)Oc2ncn(-c4ccccc4)c23)cc1 ; CC(C)(C)c1ccc(N2c3ccc(C(C)(C)C)cc3B3c4c(cccc42)Oc2ncn(-c4ccccc4)c23)cc1]

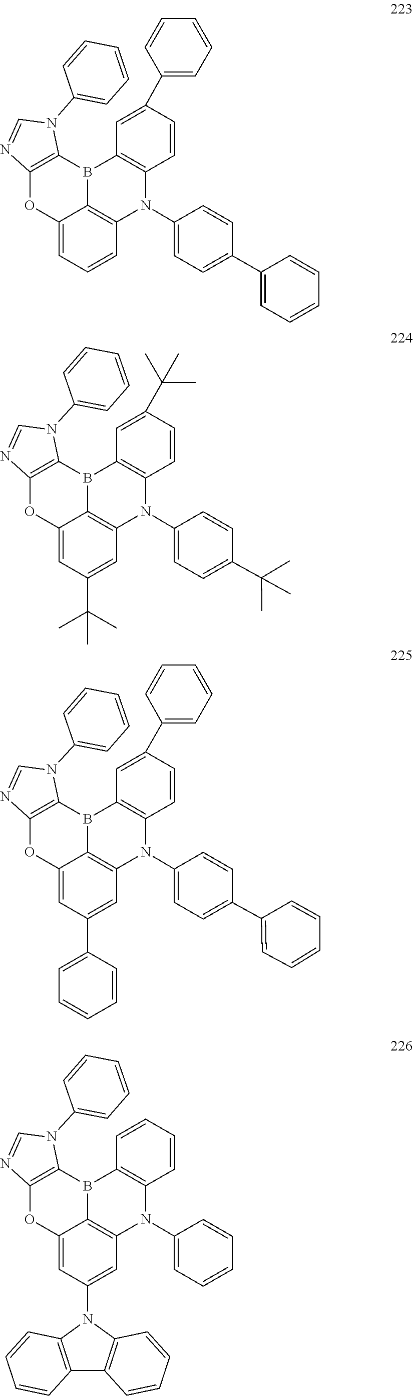

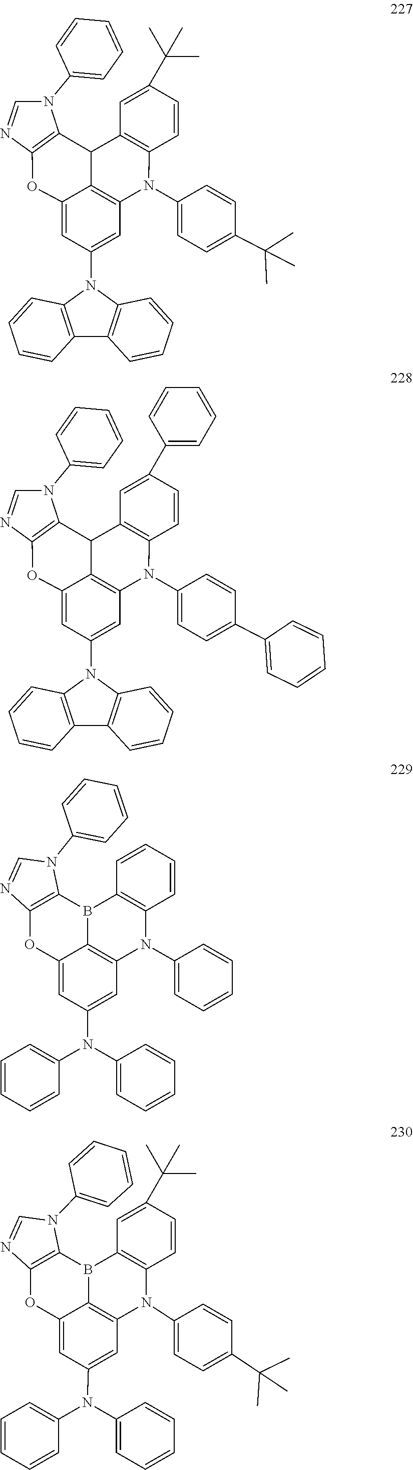

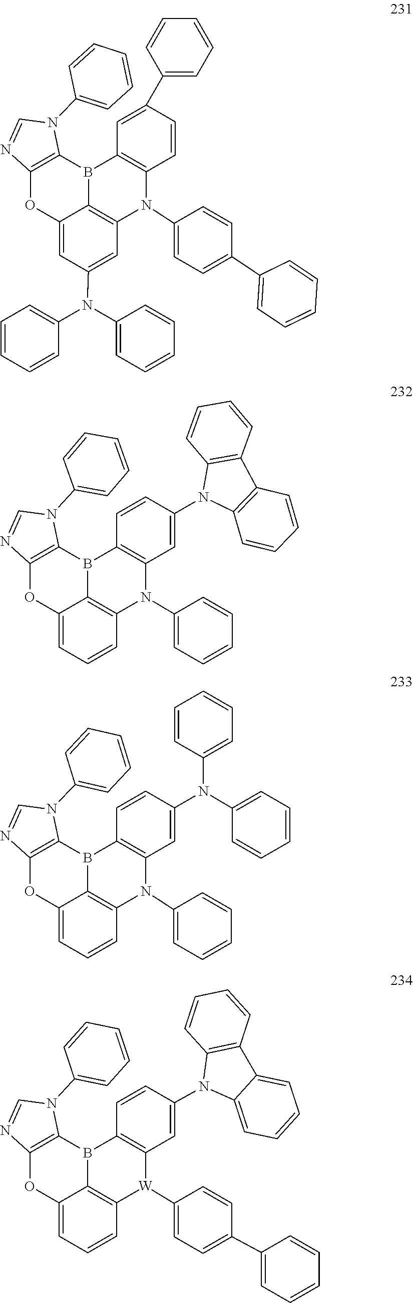

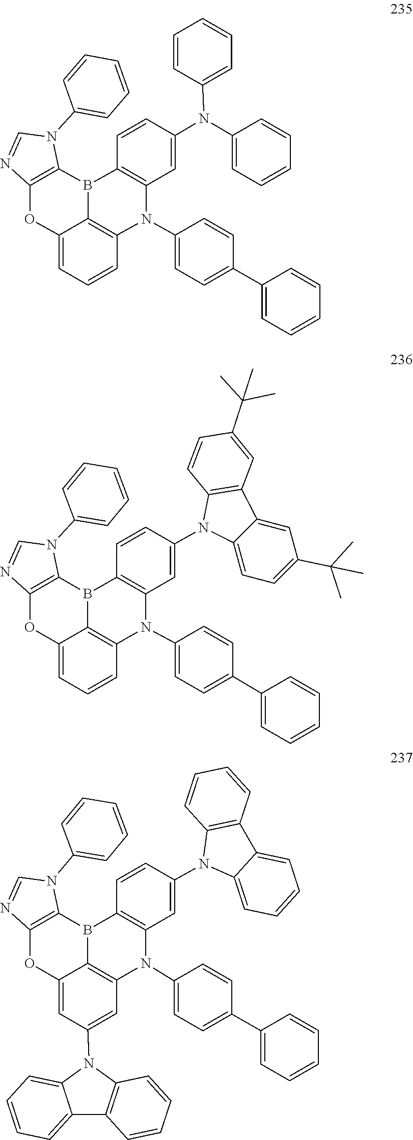 [SMILES: c1ccc(-c2ccc(N3c4cc(N(c5ccccc5)c5ccccc5)ccc4B4c5c(cccc53)Oc3ncn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(ncn1-c1ccccc1)O3 ; c1ccc(-c2ccc(N3c4cc(-n5c6ccccc6c6ccccc65)ccc4B4c5c(cc(-n6c7ccccc7c7ccccc76)cc53)Oc3ncn(-c5ccccc5)c34)cc2)cc1]

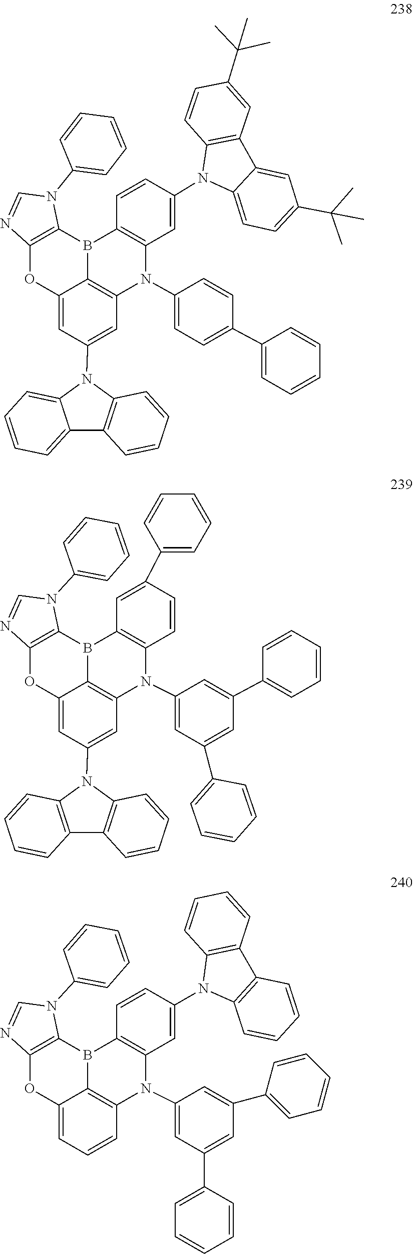

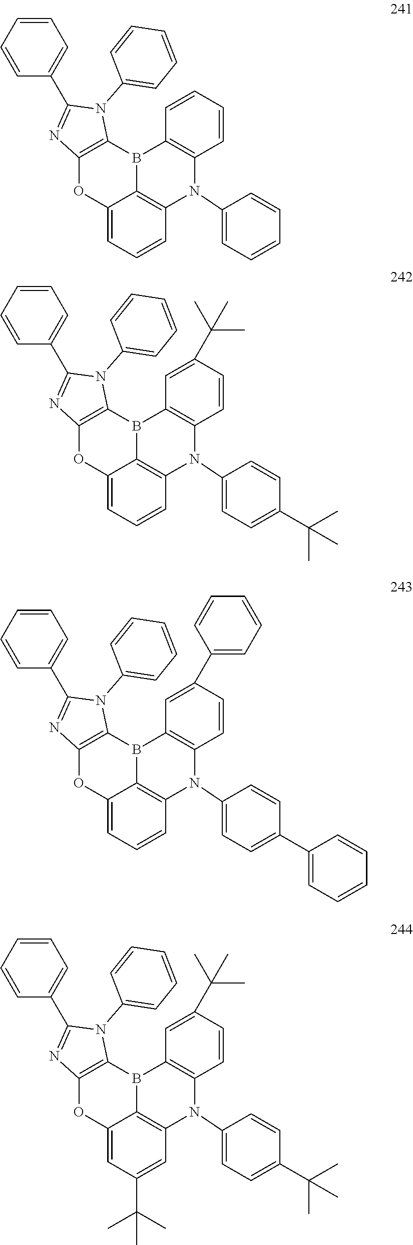

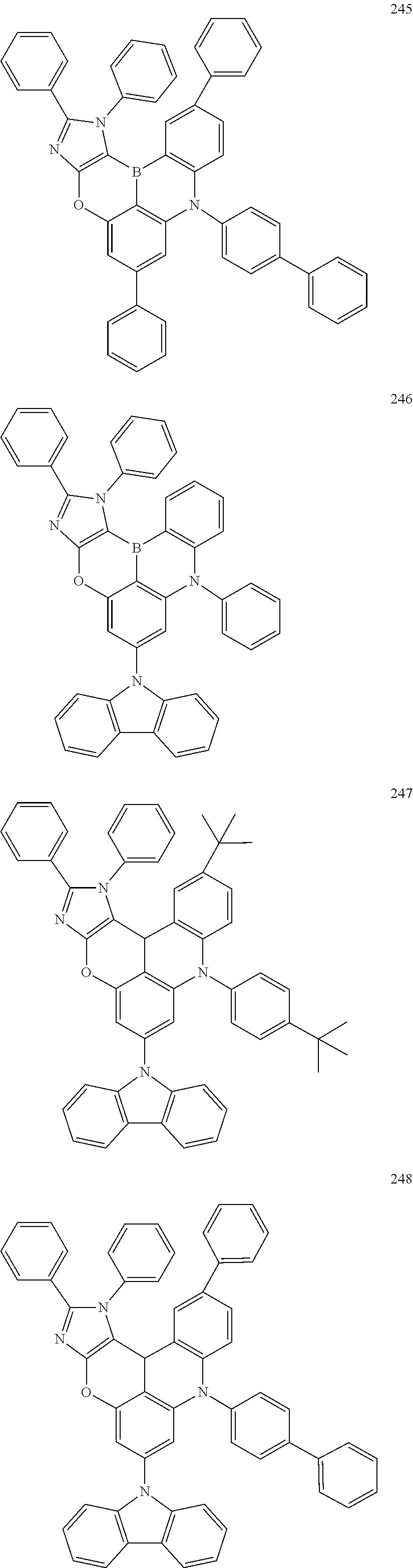

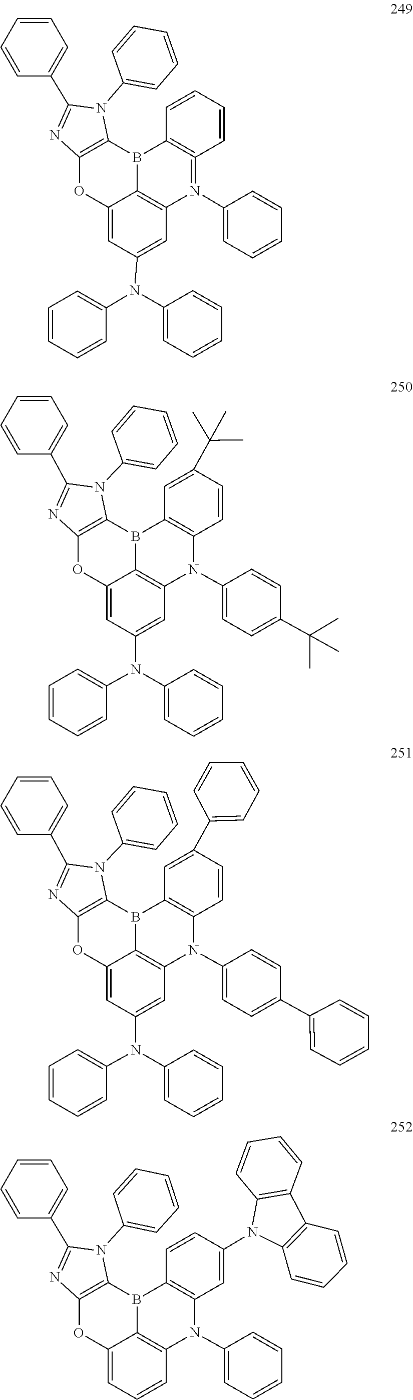

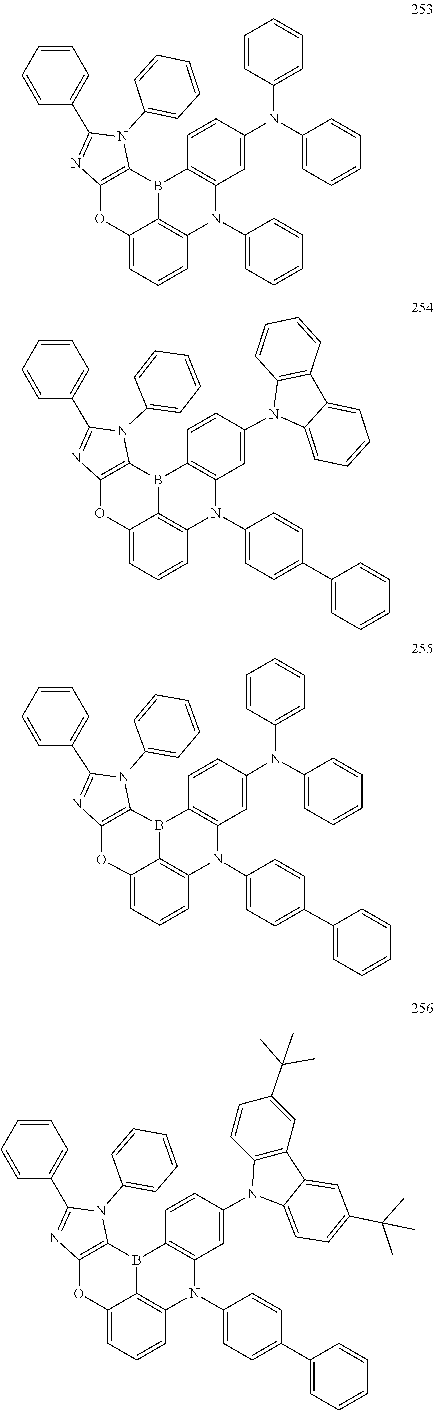

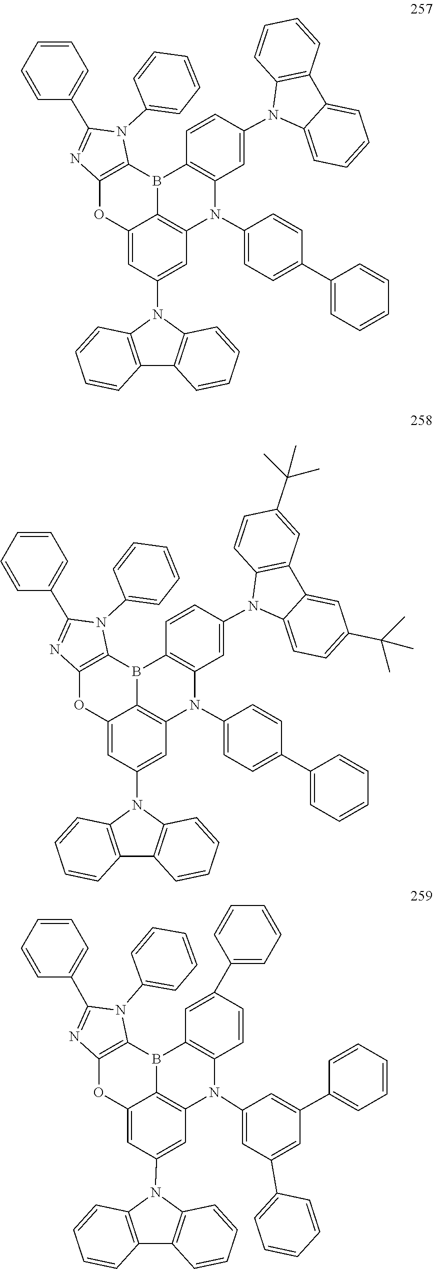

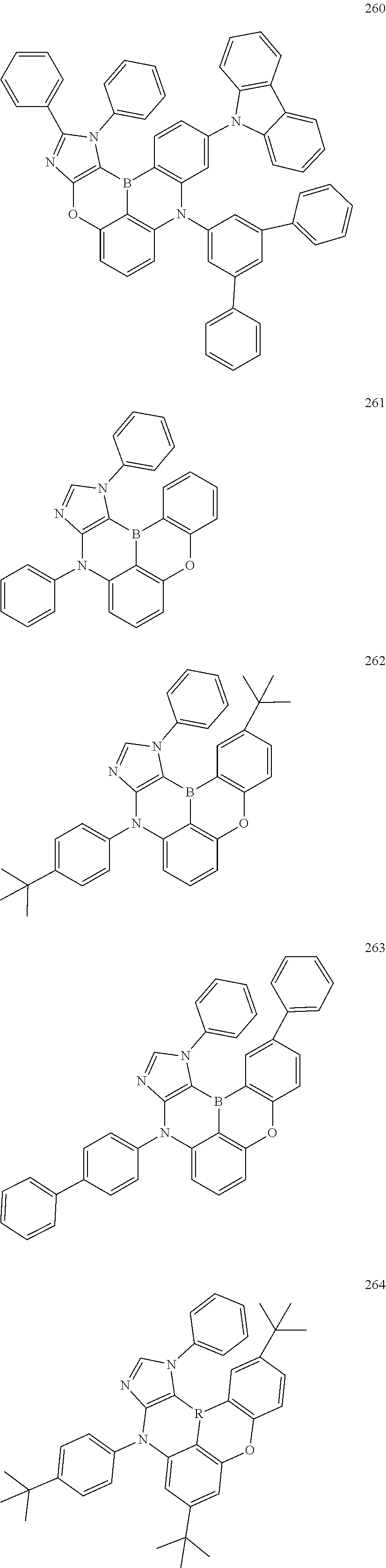

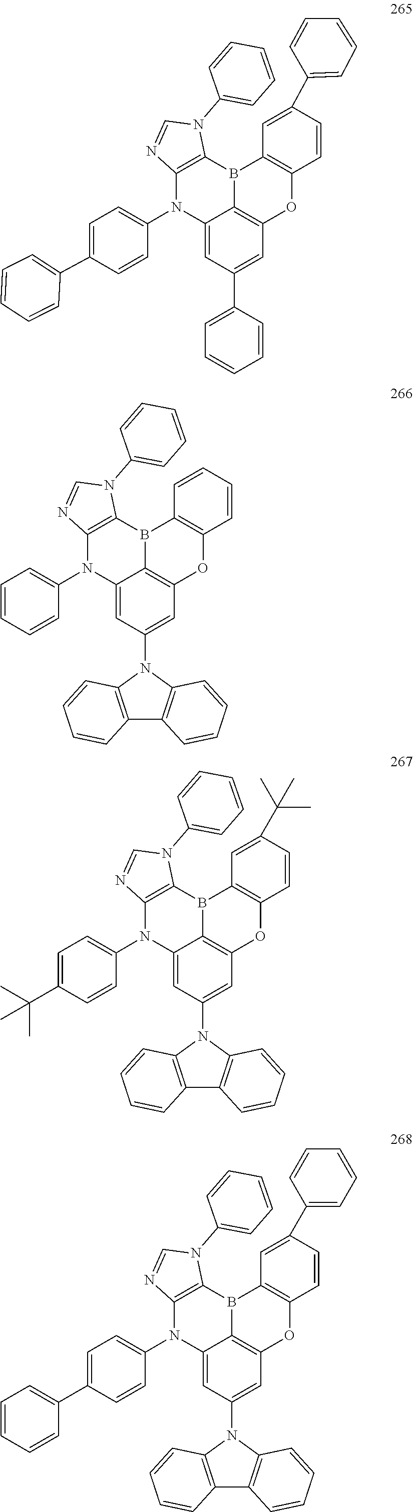

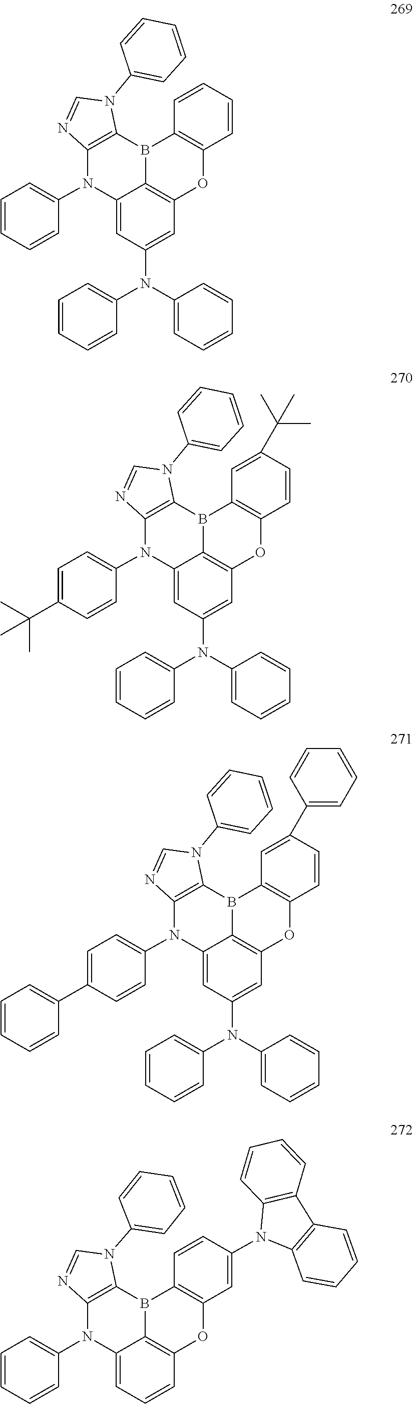

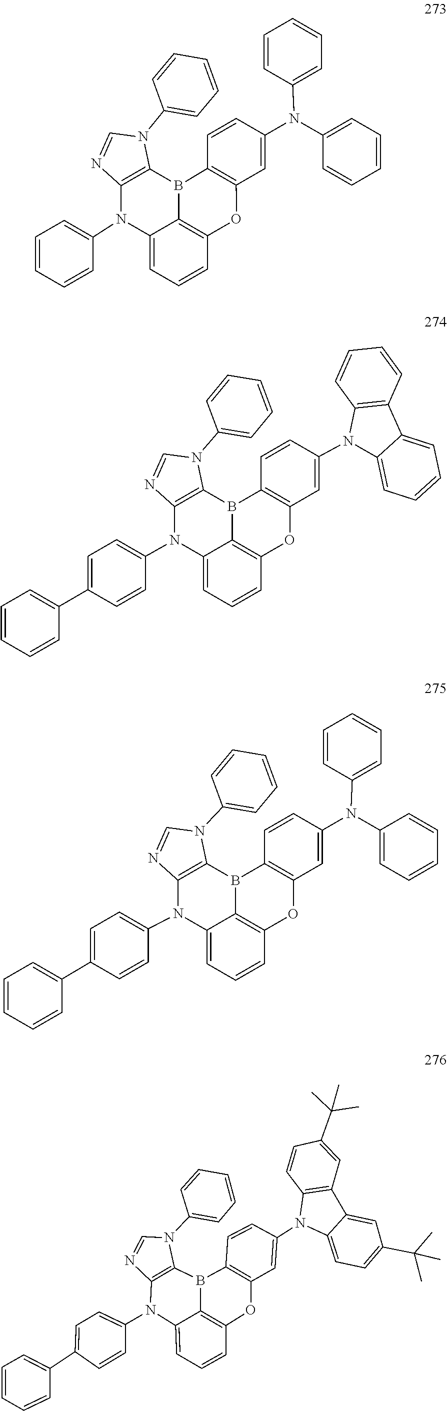 [SMILES: c1ccc(N(c2ccccc2)c2ccc3c(c2)Oc2cccc4c2B3c2c(ncn2-c2ccccc2)N4c2ccccc2)cc1 ; c1ccc(-c2ccc(N3c4cccc5c4B(c4ccc(-n6c7ccccc7c7ccccc76)cc4O5)c4c3ncn4-c3ccccc3)cc2)cc1 ; c1ccc(-c2ccc(N3c4cccc5c4B(c4ccc(N(c6ccccc6)c6ccccc6)cc4O5)c4c3ncn4-c3ccccc3)cc2)cc1 ; CC(C)(C)c1ccc2c(c1)c1cc(C(C)(C)C)ccc1n2-c1ccc2c(c1)Oc1cccc3c1B2c1c(ncn1-c1ccccc1)N3c1ccc(-c2ccccc2)cc1]

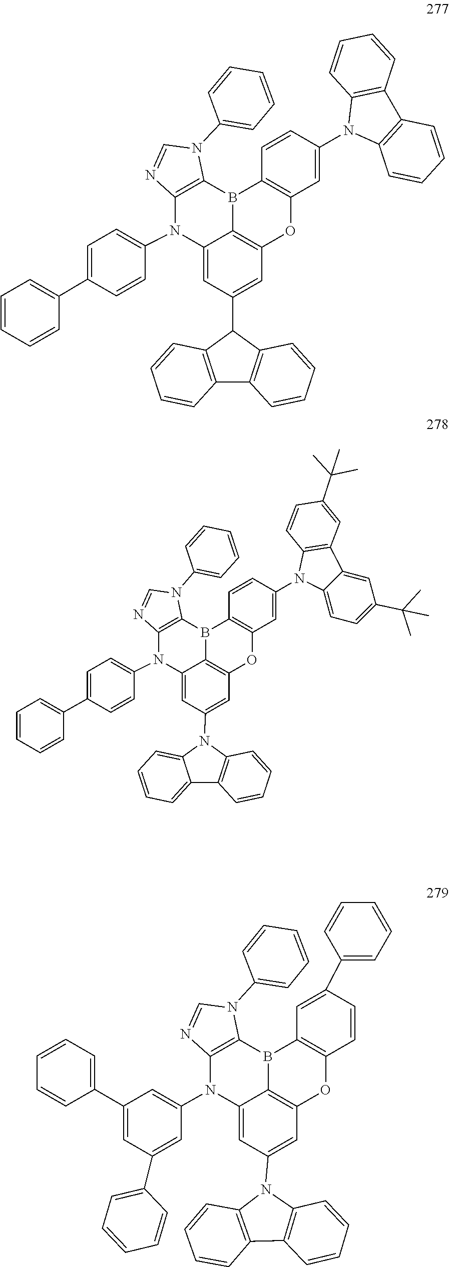 [SMILES: c1ccc(-c2ccc(N3c4cc(C5c6ccccc6-c6ccccc65)cc5c4B(c4ccc(-n6c7ccccc7c7ccccc76)cc4O5)c4c3ncn4-c3ccccc3)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1c(ncn1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3ncn4-c3ccccc3)c2)cc1]

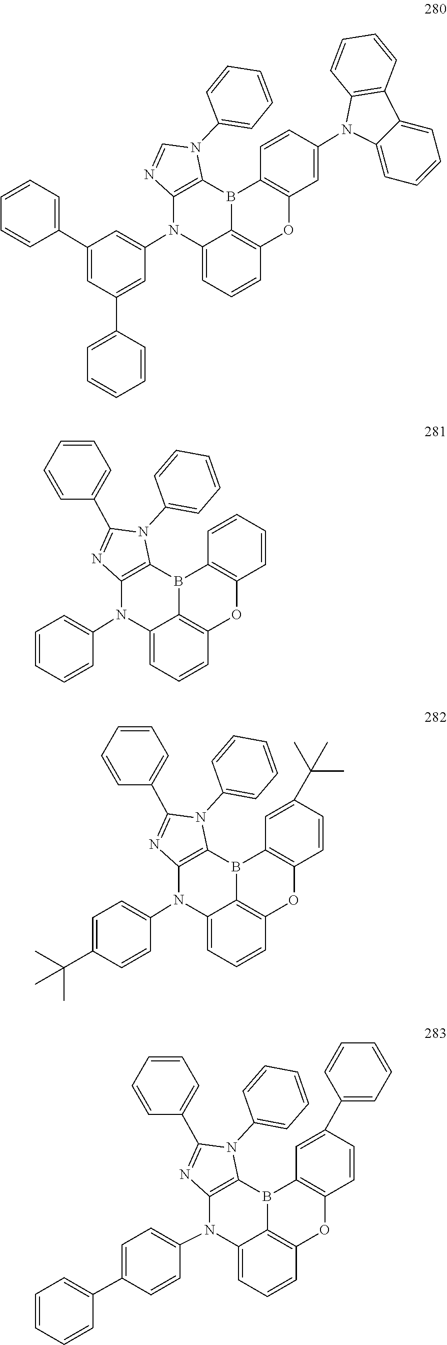 [SMILES: c1ccc(-c2cc(-c3ccccc3)cc(N3c4cccc5c4B(c4ccc(-n6c7ccccc7c7ccccc76)cc4O5)c4c3ncn4-c3ccccc3)c2)cc1 ; c1ccc(-c2nc3c(n2-c2ccccc2)B2c4ccccc4Oc4cccc(c42)N3c2ccccc2)cc1 ; CC(C)(C)c1ccc(N2c3cccc4c3B(c3cc(C(C)(C)C)ccc3O4)c3c2nc(-c2ccccc2)n3-c2ccccc2)cc1 ; c1ccc(-c2ccc(N3c4cccc5c4B(c4cc(-c6ccccc6)ccc4O5)c4c3nc(-c3ccccc3)n4-c3ccccc3)cc2)cc1]

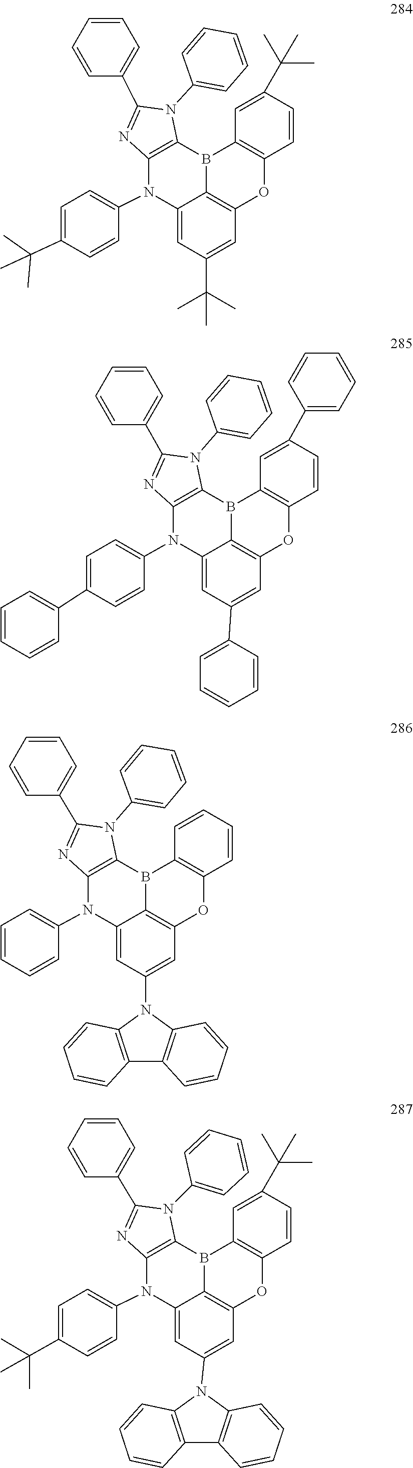

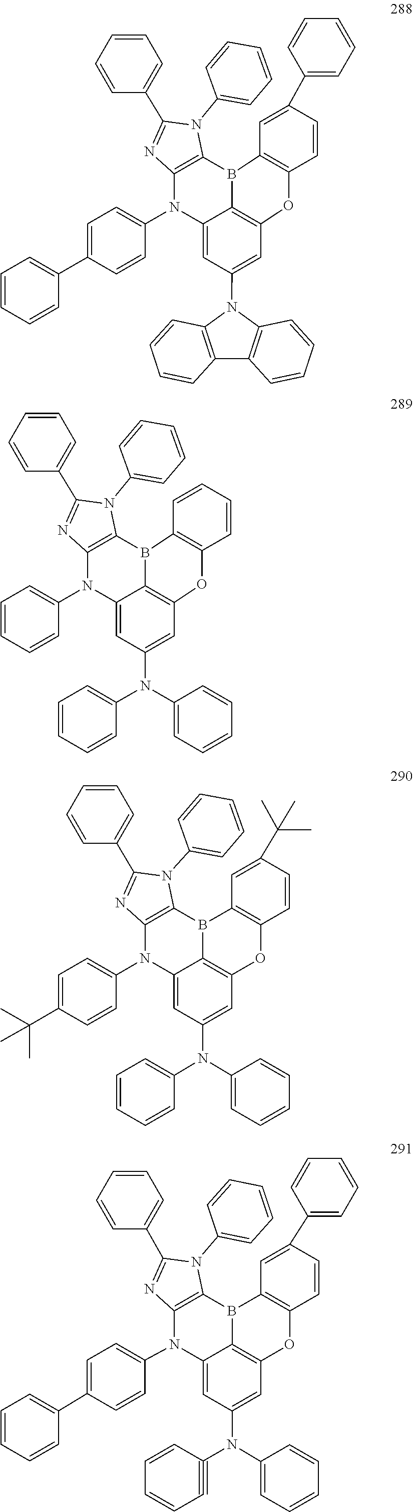

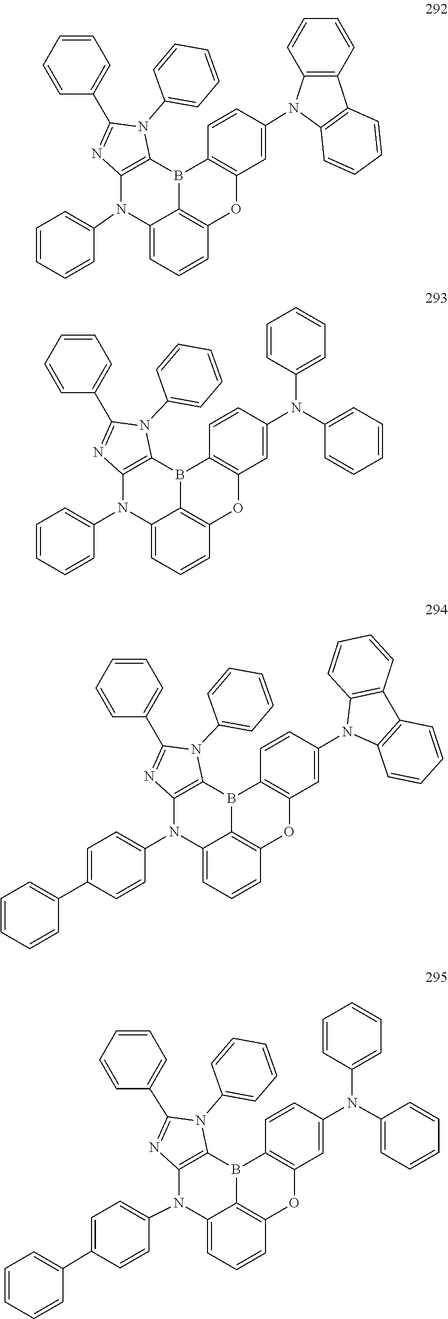 [SMILES: c1ccc(-c2nc3c(n2-c2ccccc2)B2c4ccc(-n5c6ccccc6c6ccccc65)cc4Oc4cccc(c42)N3c2ccccc2)cc1 ; c1ccc(-c2nc3c(n2-c2ccccc2)B2c4ccc(N(c5ccccc5)c5ccccc5)cc4Oc4cccc(c42)N3c2ccccc2)cc1 ; c1ccc(-c2ccc(N3c4cccc5c4B(c4ccc(-n6c7ccccc7c7ccccc76)cc4O5)c4c3nc(-c3ccccc3)n4-c3ccccc3)cc2)cc1 ; c1ccc(-c2ccc(N3c4cccc5c4B(c4ccc(N(c6ccccc6)c6ccccc6)cc4O5)c4c3nc(-c3ccccc3)n4-c3ccccc3)cc2)cc1]

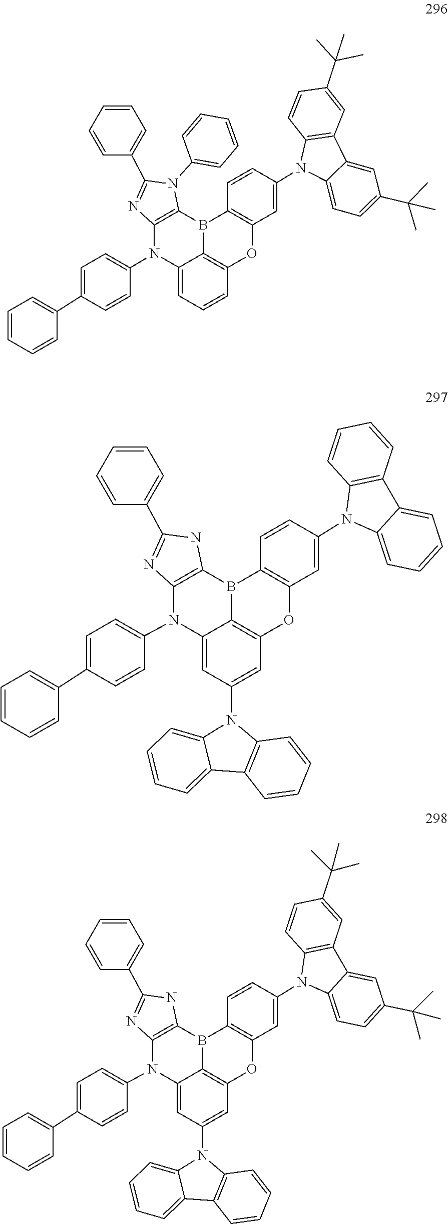 [SMILES: CC(C)(C)c1ccc2c(c1)c1cc(C(C)(C)C)ccc1n2-c1ccc2c(c1)Oc1cccc3c1B2c1c(nc(-c2ccccc2)n1-c1ccccc1)N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4[nH]c(-c5ccccc5)nc43)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1[nH]c(-c2ccccc2)nc1N3c1ccc(-c2ccccc2)cc1]

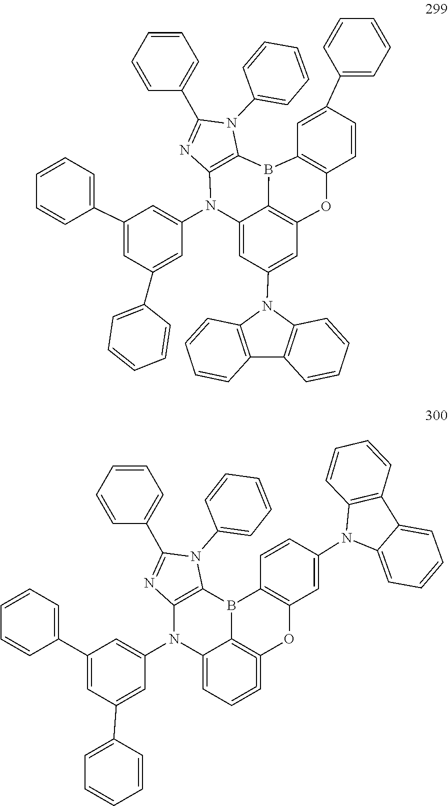

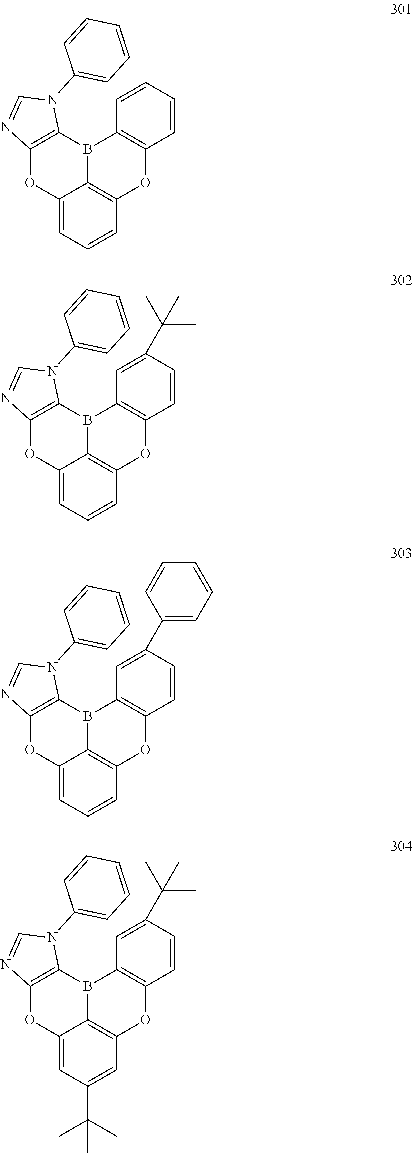

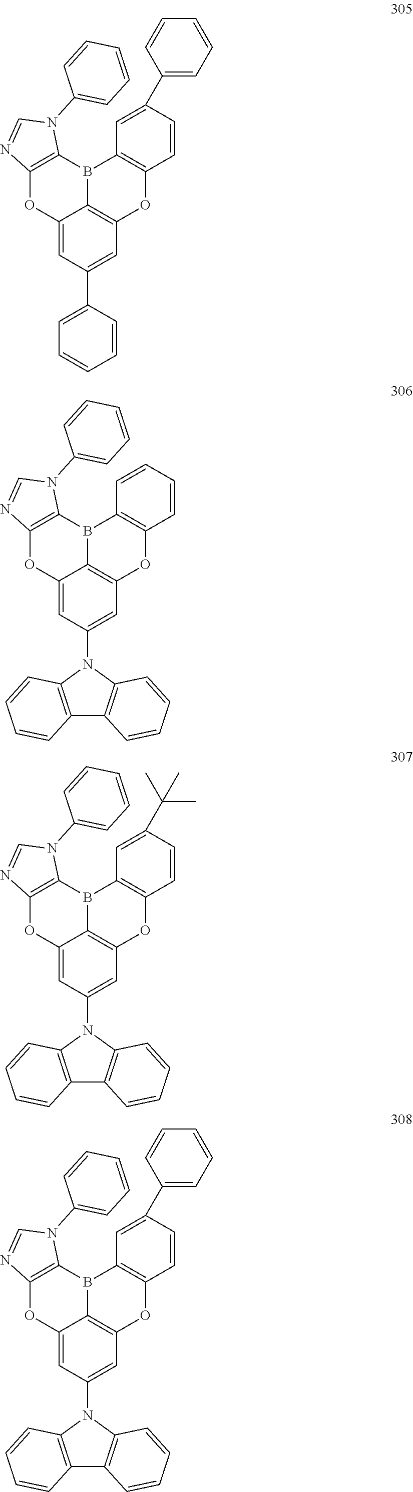

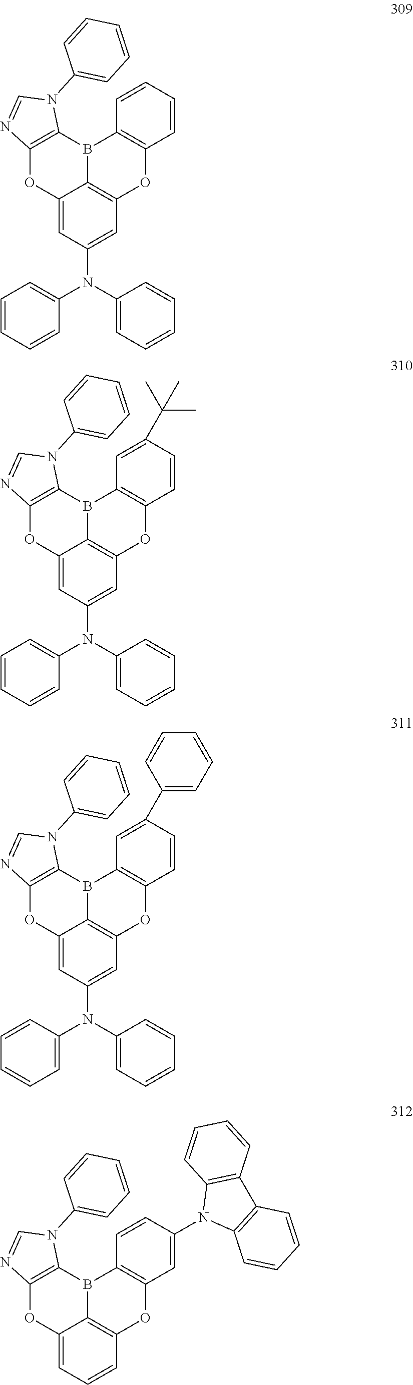

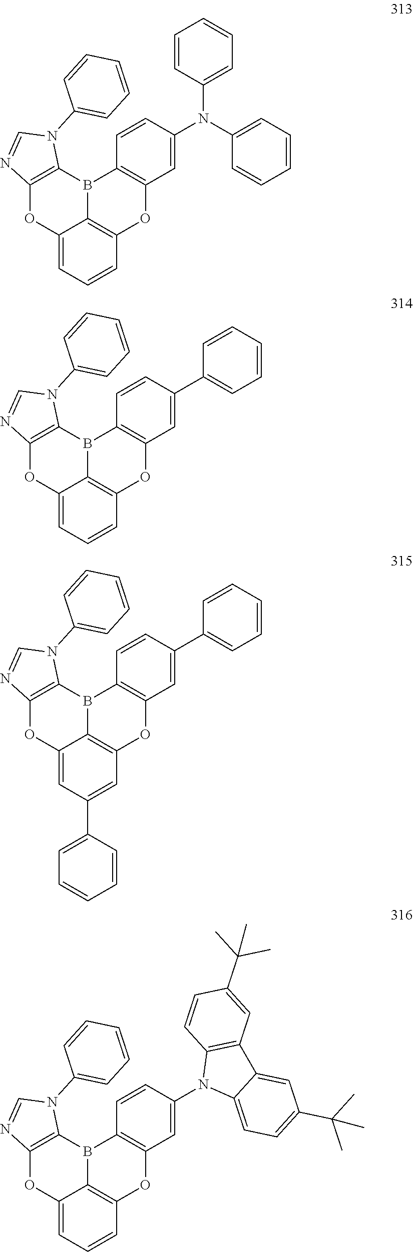

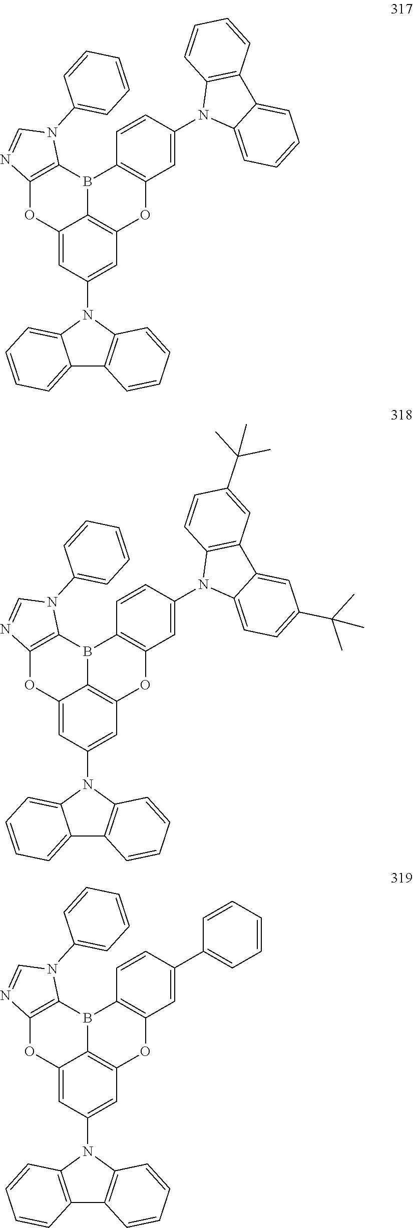

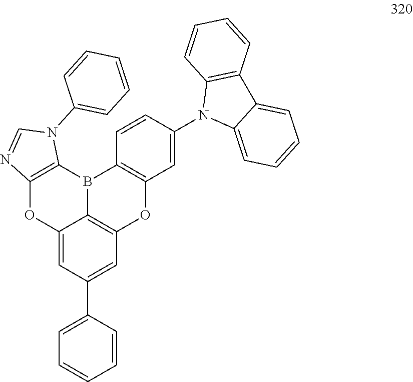 [SMILES: c1ccc(-c2cc3c4c(c2)Oc2ncn(-c5ccccc5)c2B4c2ccc(-n4c5ccccc5c5ccccc54)cc2O3)cc1]

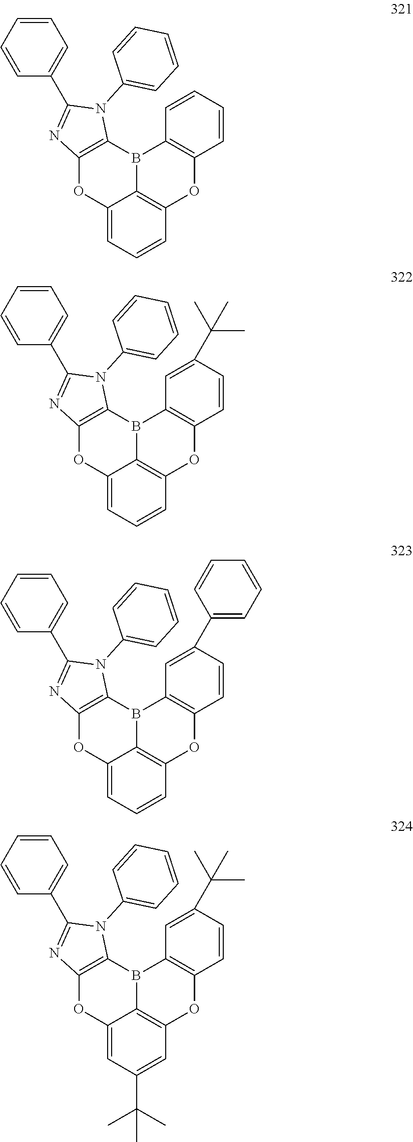

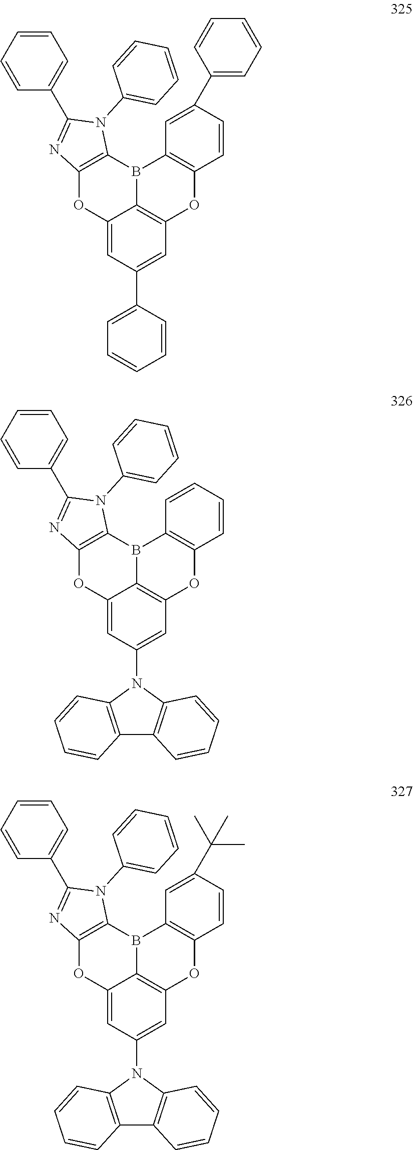 [SMILES: c1ccc(-c2cc3c4c(c2)Oc2nc(-c5ccccc5)n(-c5ccccc5)c2B4c2cc(-c4ccccc4)ccc2O3)cc1 ; c1ccc(-c2nc3c(n2-c2ccccc2)B2c4ccccc4Oc4cc(-n5c6ccccc6c6ccccc65)cc(c42)O3)cc1 ; CC(C)(C)c1ccc2c(c1)B1c3c(cc(-n4c5ccccc5c5ccccc54)cc3Oc3nc(-c4ccccc4)n(-c4ccccc4)c31)O2]

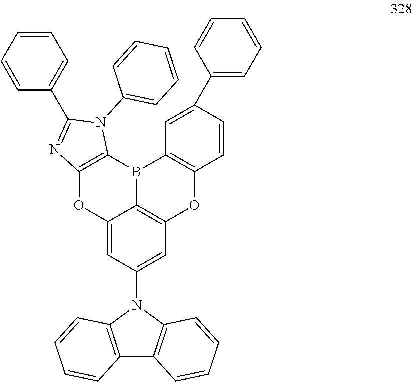 [SMILES: c1ccc(-c2ccc3c(c2)B2c4c(cc(-n5c6ccccc6c6ccccc65)cc4Oc4nc(-c5ccccc5)n(-c5ccccc5)c42)O3)cc1]

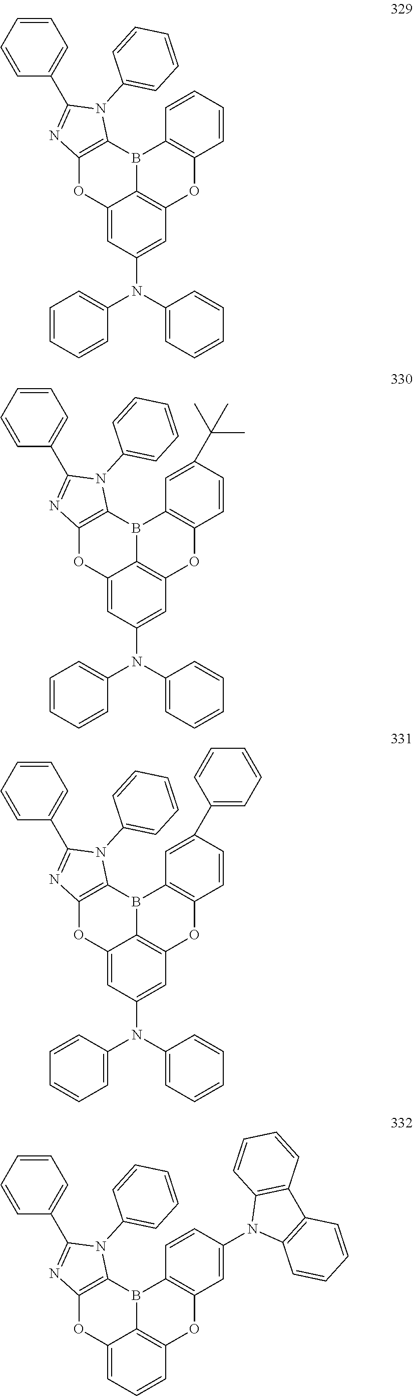

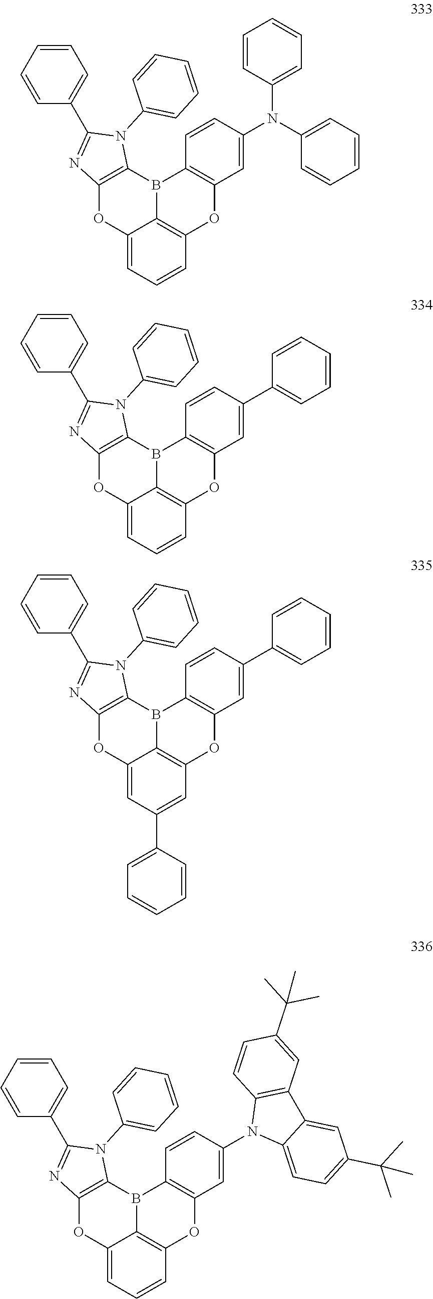

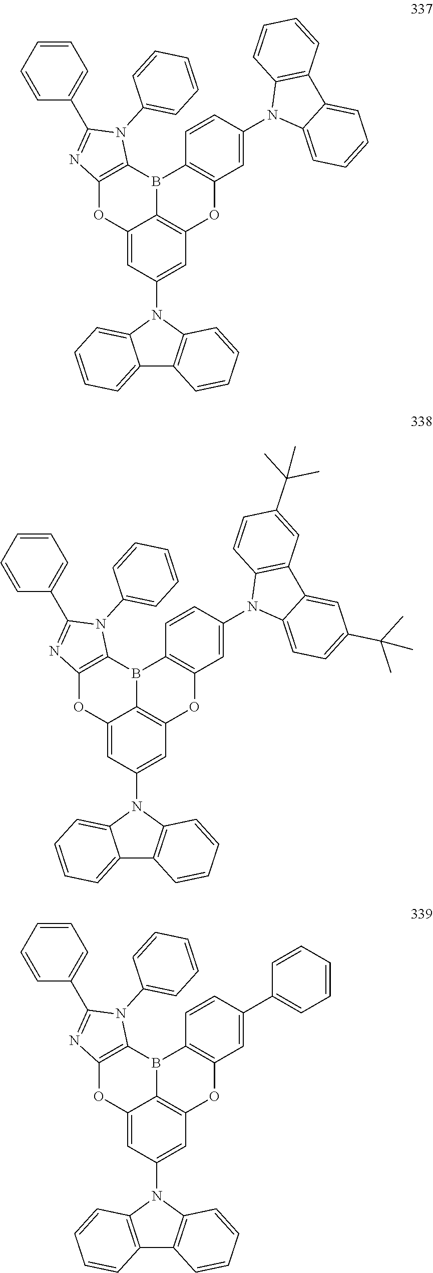 [SMILES: c1ccc(-c2nc3c(n2-c2ccccc2)B2c4ccc(-n5c6ccccc6c6ccccc65)cc4Oc4cc(-n5c6ccccc6c6ccccc65)cc(c42)O3)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1c(nc(-c2ccccc2)n1-c1ccccc1)O3 ; c1ccc(-c2ccc3c(c2)Oc2cc(-n4c5ccccc5c5ccccc54)cc4c2B3c2c(nc(-c3ccccc3)n2-c2ccccc2)O4)cc1]

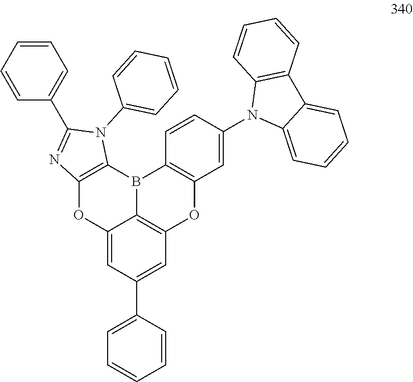 [SMILES: c1ccc(-c2cc3c4c(c2)Oc2nc(-c5ccccc5)n(-c5ccccc5)c2B4c2ccc(-n4c5ccccc5c5ccccc54)cc2O3)cc1]

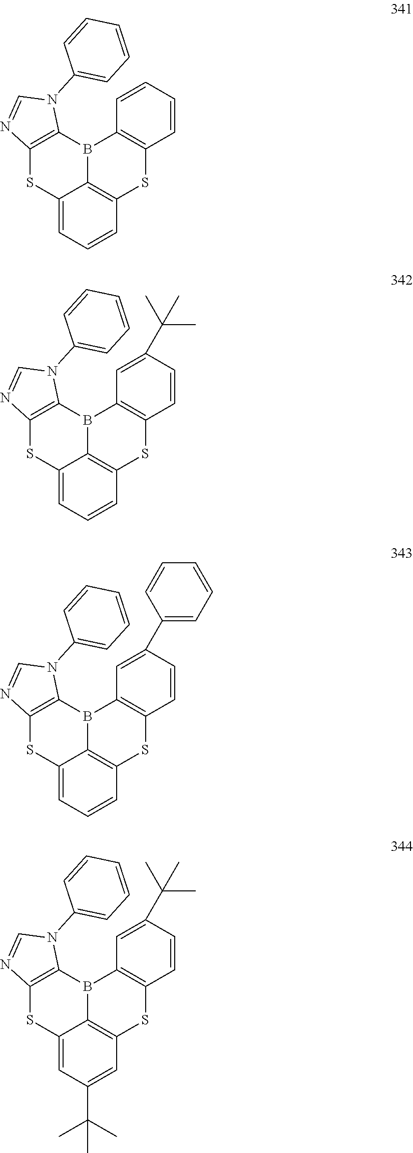

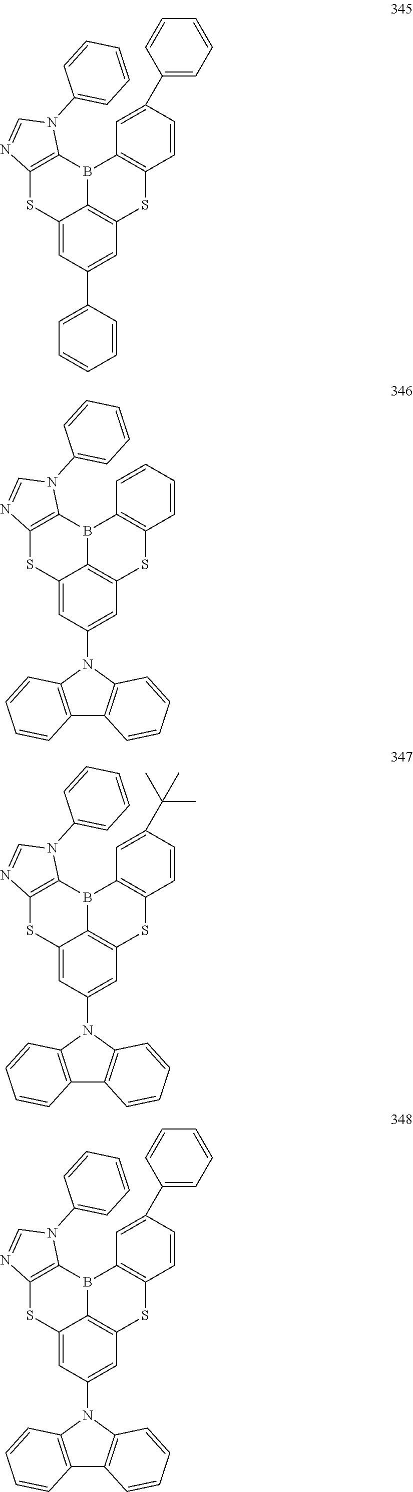

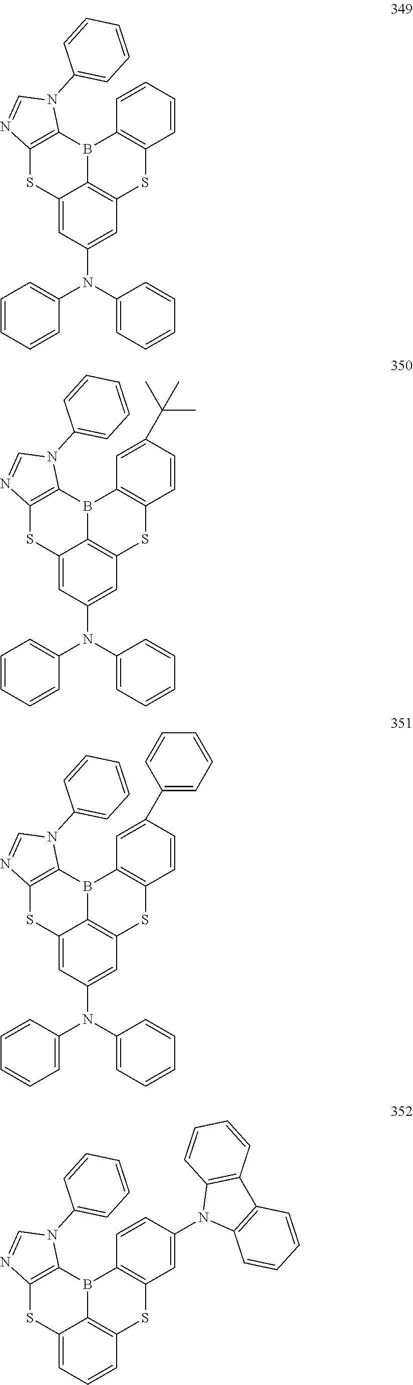

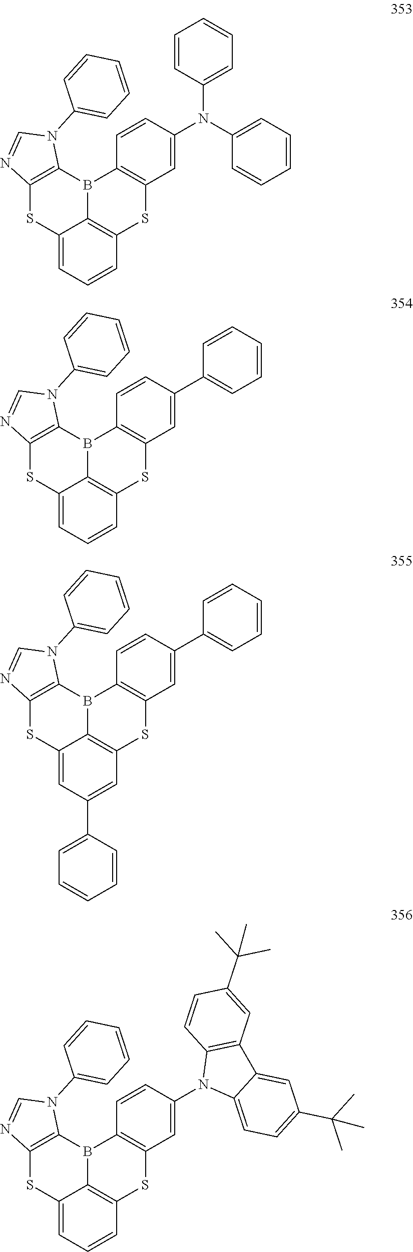

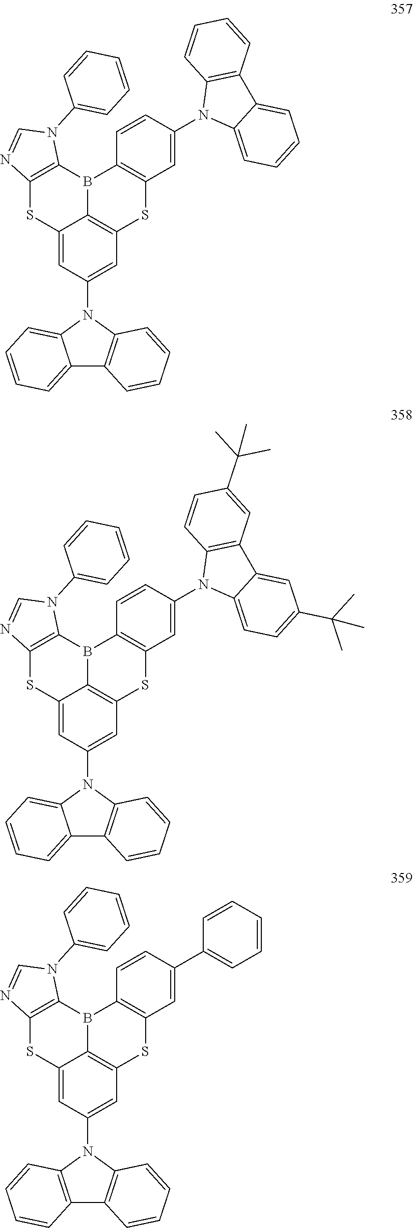

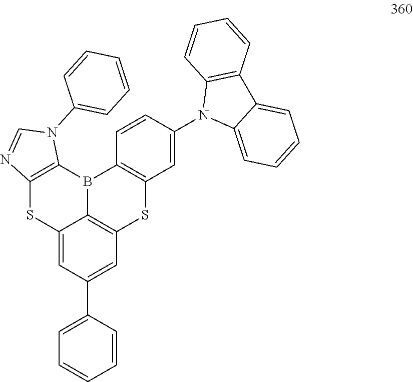 [SMILES: c1ccc(-c2cc3c4c(c2)Sc2ncn(-c5ccccc5)c2B4c2ccc(-n4c5ccccc5c5ccccc54)cc2S3)cc1]

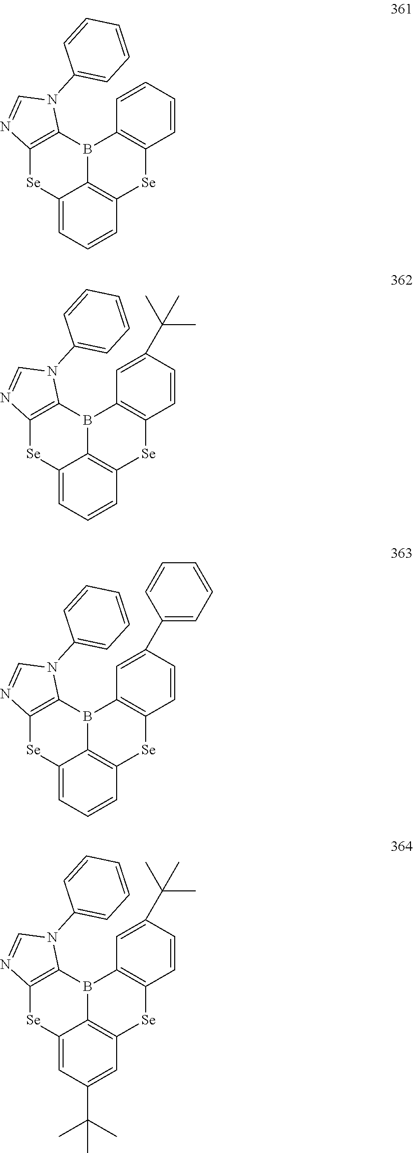

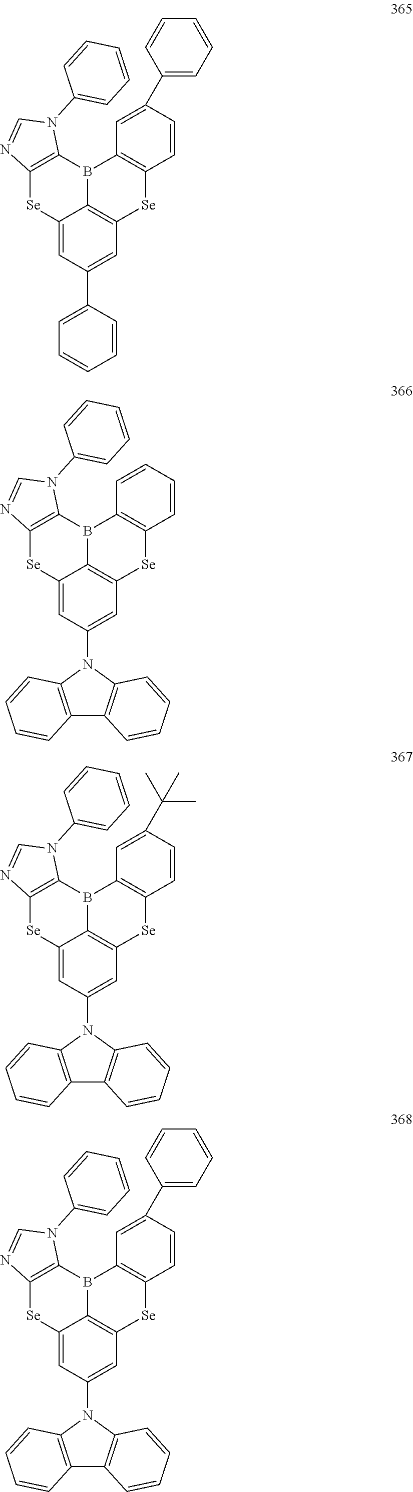

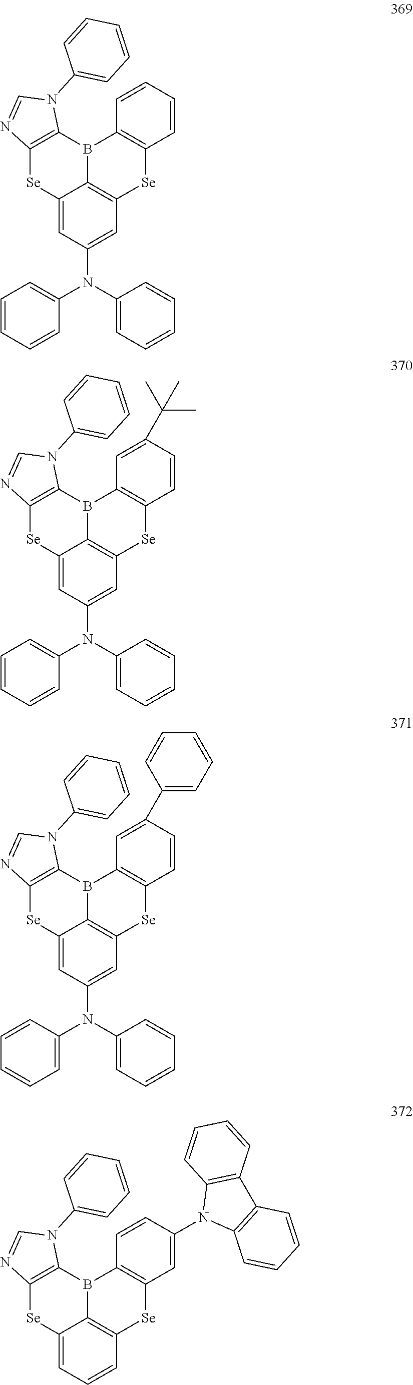

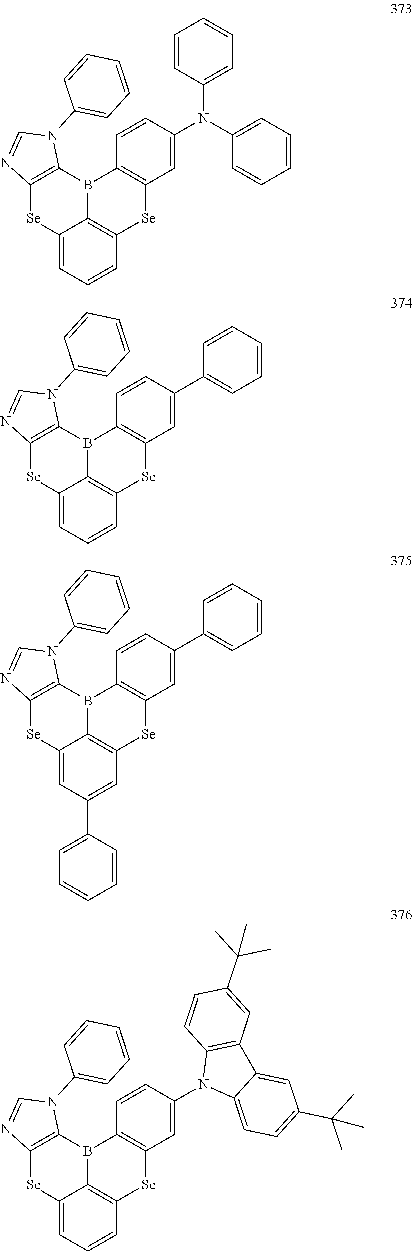

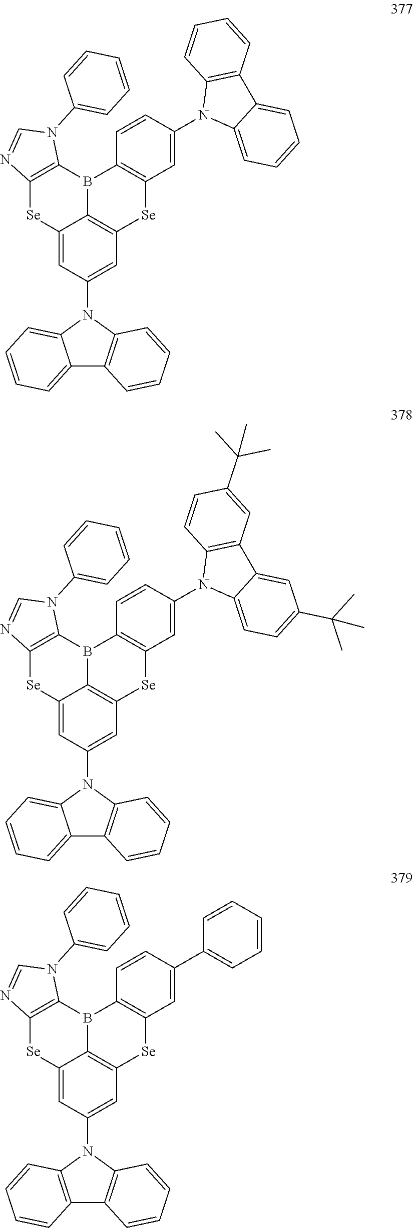

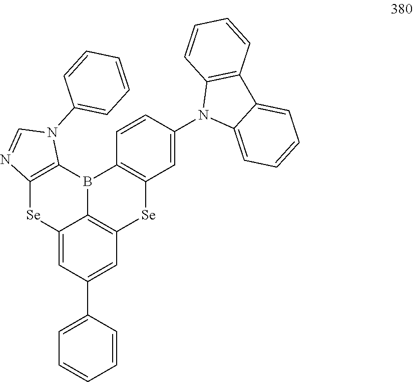 [SMILES: c1ccc(-c2cc3c4c(c2)[Se]c2ncn(-c5ccccc5)c2B4c2ccc(-n4c5ccccc5c5ccccc54)cc2[Se]3)cc1]

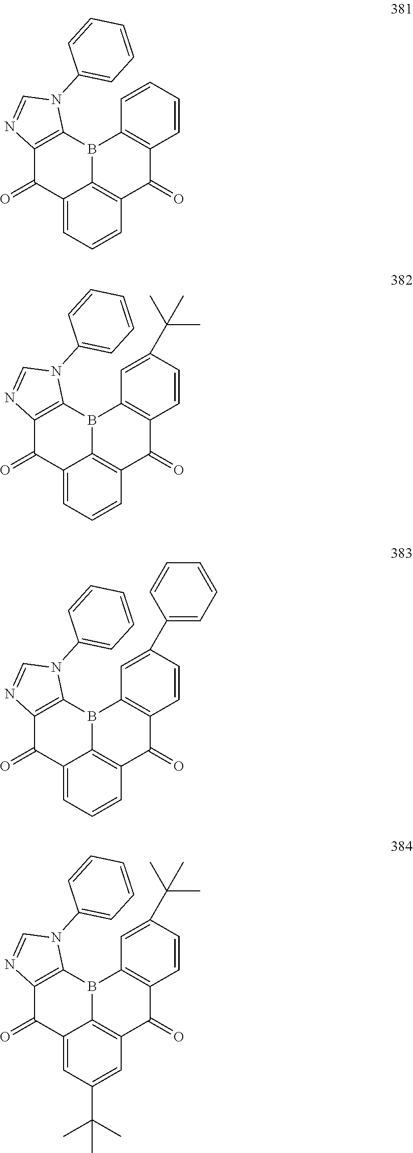

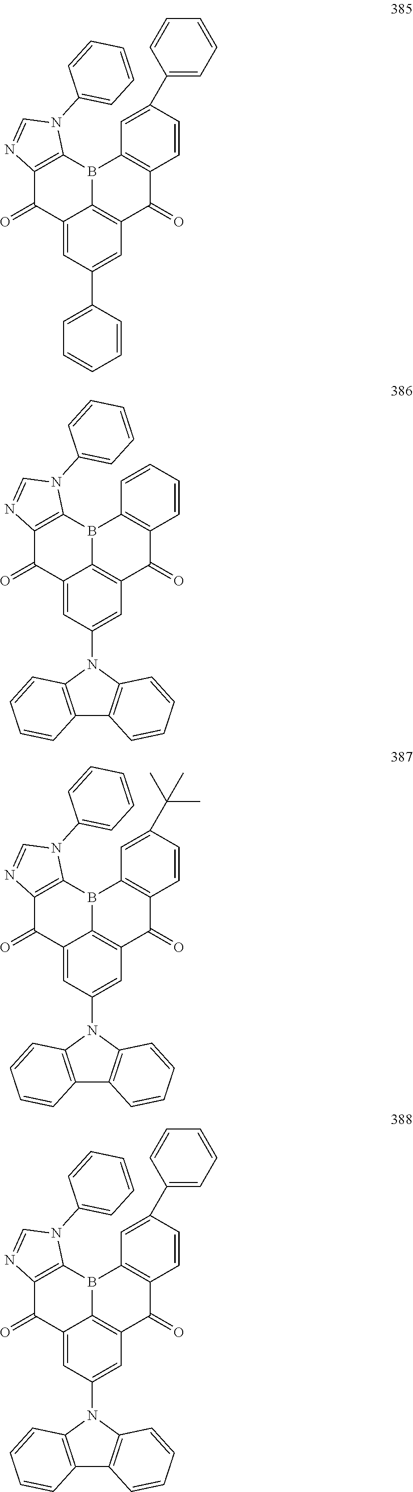

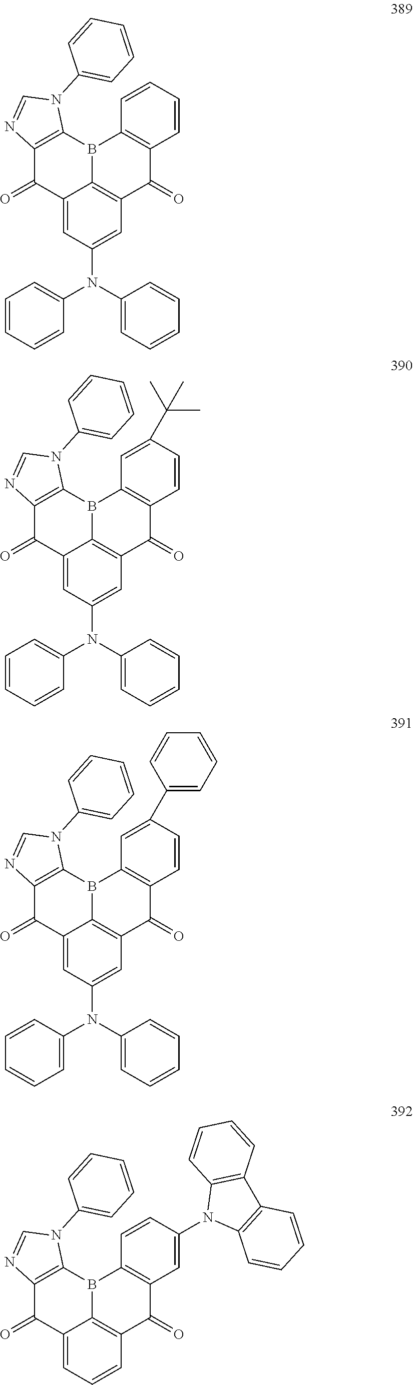

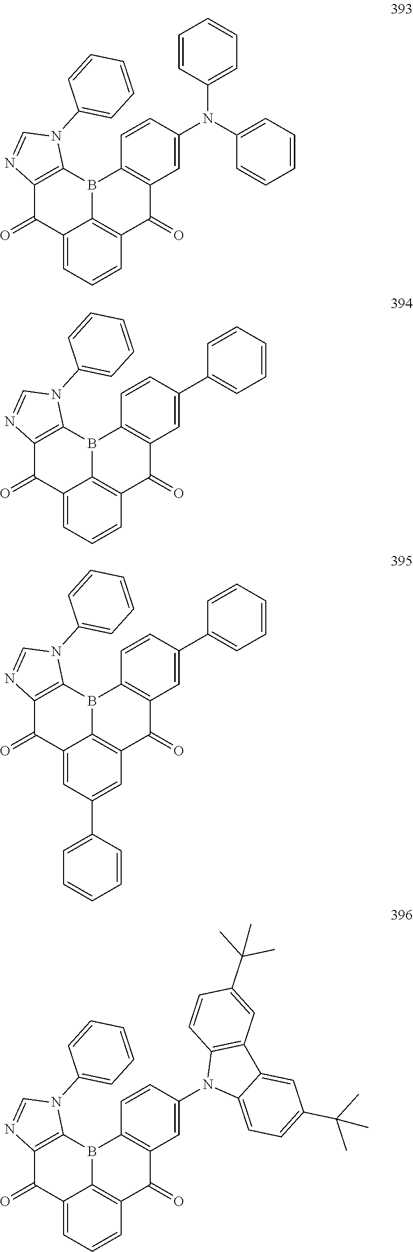

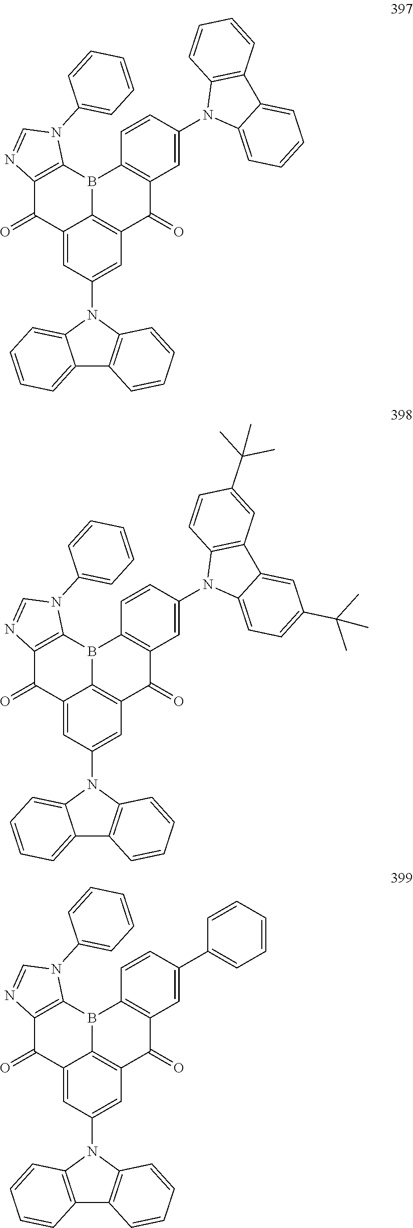

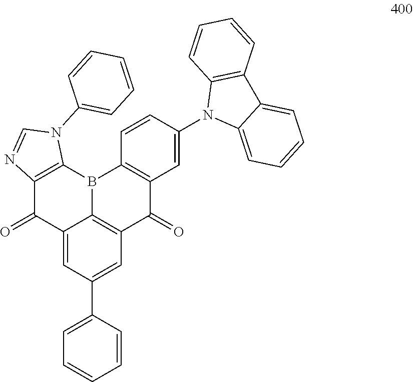 [SMILES: O=C1c2cc(-n3c4ccccc4c4ccccc43)ccc2B2c3c1cc(-c1ccccc1)cc3C(=O)c1ncn(-c3ccccc3)c12]

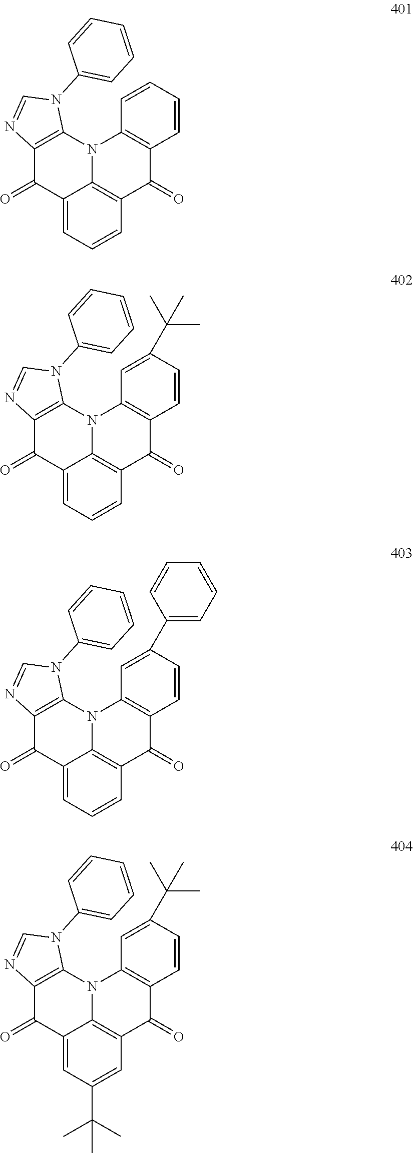

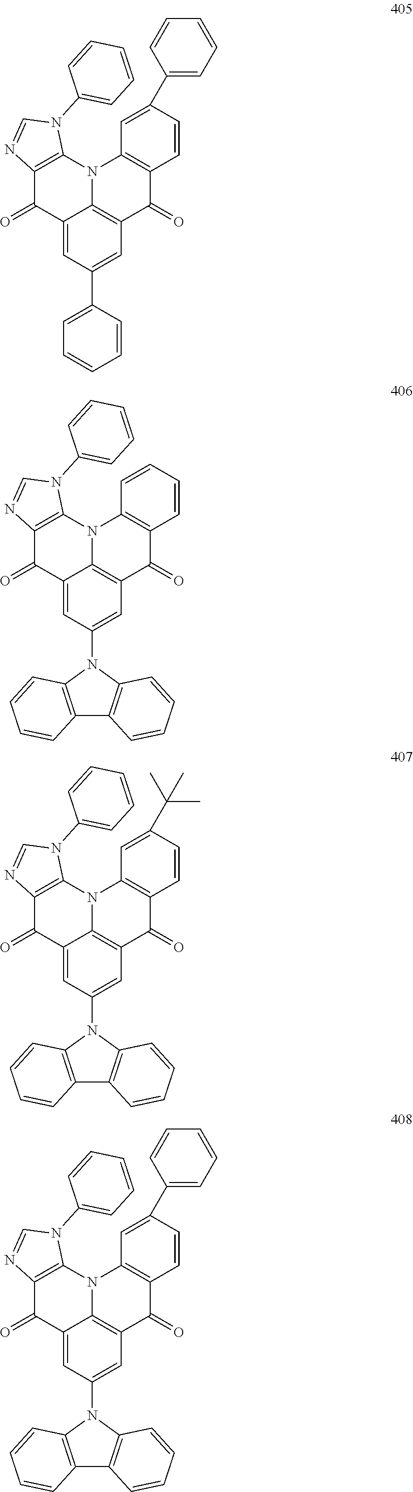

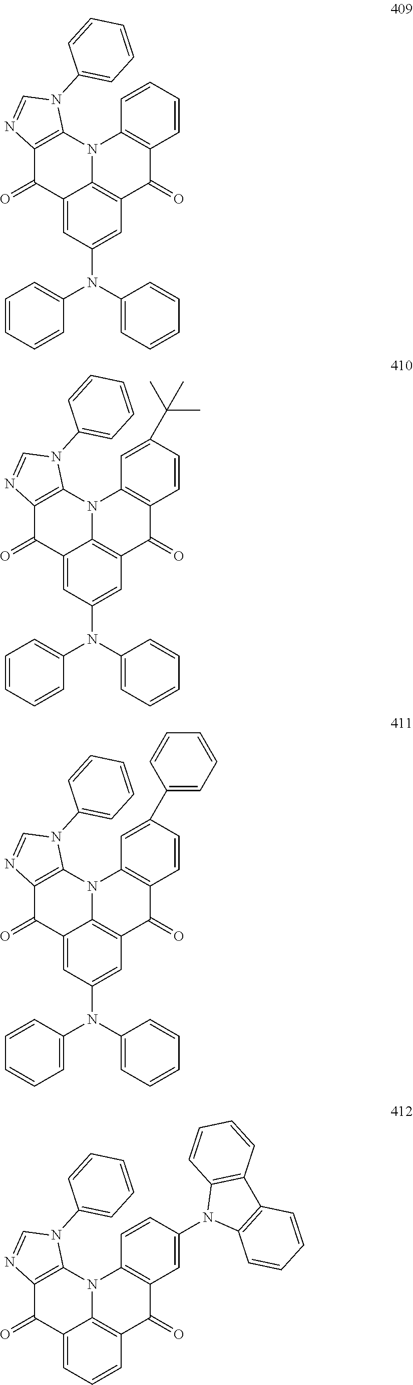

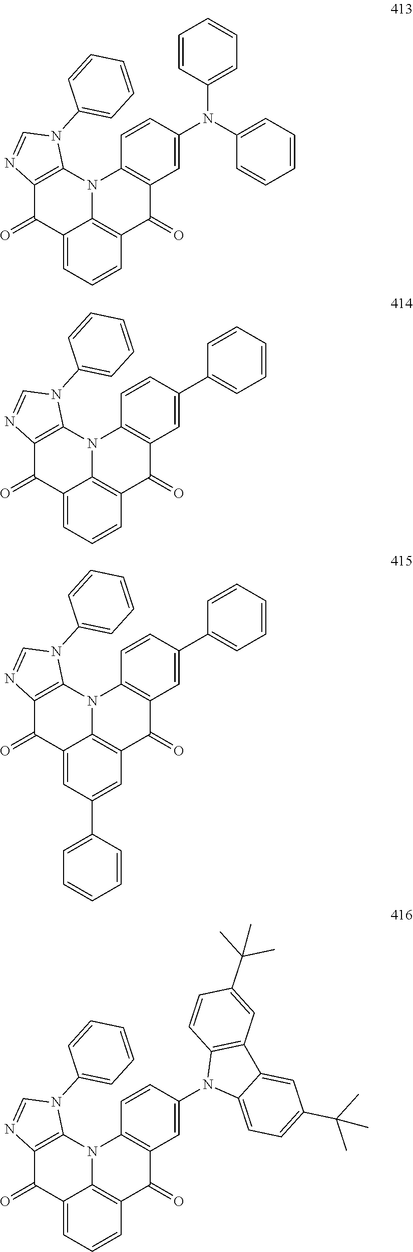

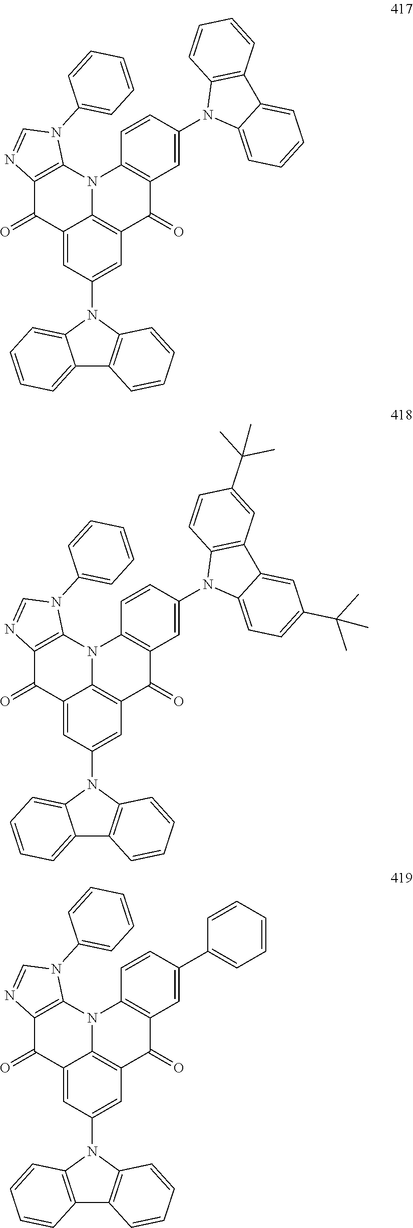

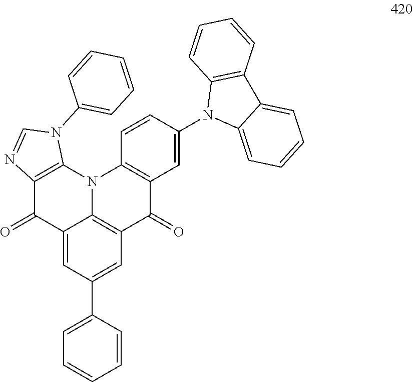 [SMILES: O=c1c2cc(-n3c4ccccc4c4ccccc43)ccc2n2c3c1cc(-c1ccccc1)cc3c(=O)c1ncn(-c3ccccc3)c12]

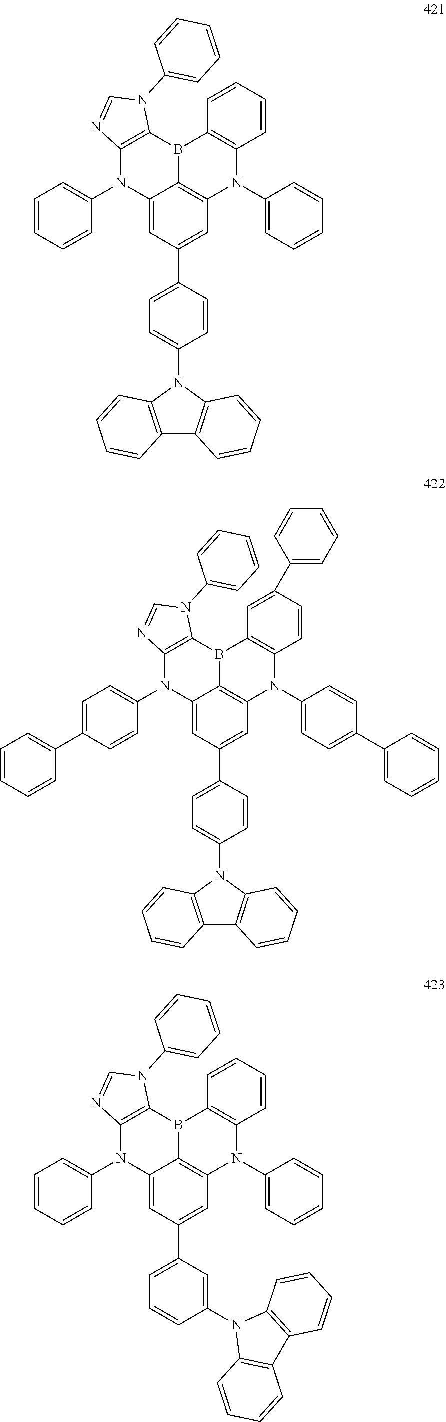 [SMILES: c1ccc(N2c3ccccc3B3c4c2cc(-c2ccc(-n5c6ccccc6c6ccccc65)cc2)cc4N(c2ccccc2)c2ncn(-c4ccccc4)c23)cc1 ; c1ccc(-c2ccc(N3c4ccc(-c5ccccc5)cc4B4c5c3cc(-c3ccc(-n6c7ccccc7c7ccccc76)cc3)cc5N(c3ccc(-c5ccccc5)cc3)c3ncn(-c5ccccc5)c34)cc2)cc1 ; c1ccc(N2c3ccccc3B3c4c2cc(-c2cccc(-n5c6ccccc6c6ccccc65)c2)cc4N(c2ccccc2)c2ncn(-c4ccccc4)c23)cc1]

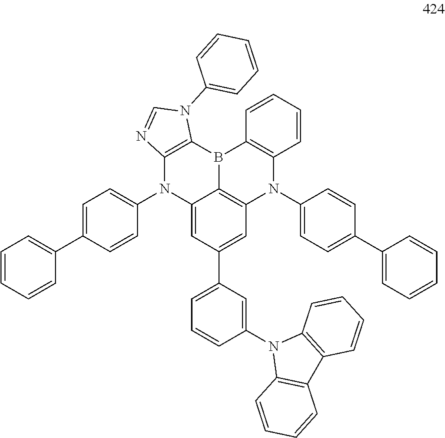 [SMILES: c1ccc(-c2ccc(N3c4ccccc4B4c5c3cc(-c3cccc(-n6c7ccccc7c7ccccc76)c3)cc5N(c3ccc(-c5ccccc5)cc3)c3ncn(-c5ccccc5)c34)cc2)cc1]

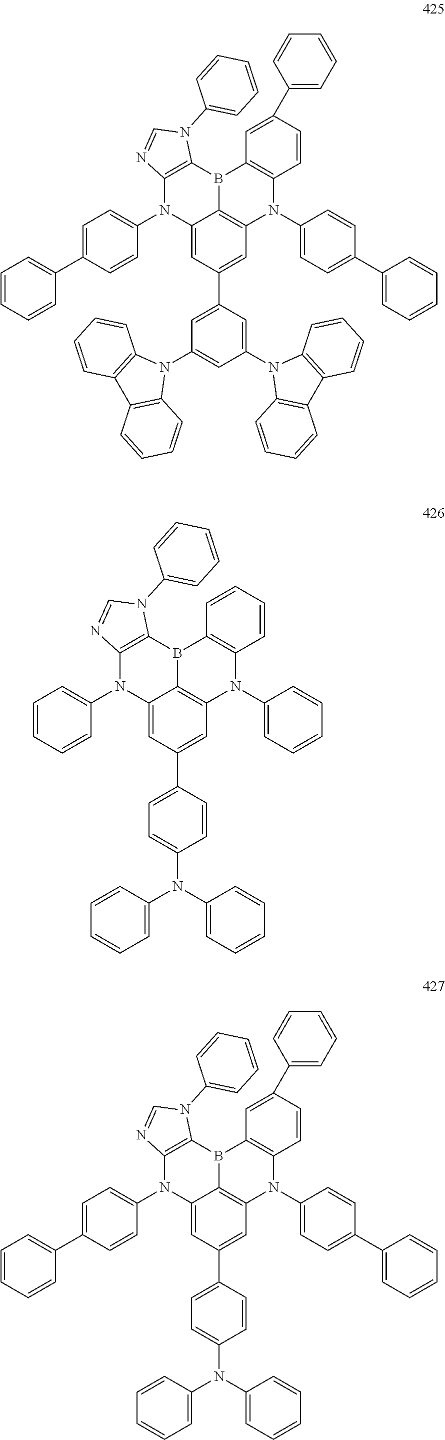 [SMILES: c1ccc(-c2ccc(N3c4ccc(-c5ccccc5)cc4B4c5c3cc(-c3cc(-n6c7ccccc7c7ccccc76)cc(-n6c7ccccc7c7ccccc76)c3)cc5N(c3ccc(-c5ccccc5)cc3)c3ncn(-c5ccccc5)c34)cc2)cc1 ; c1ccc(N(c2ccccc2)c2ccc(-c3cc4c5c(c3)N(c3ccccc3)c3ncn(-c6ccccc6)c3B5c3ccccc3N4c3ccccc3)cc2)cc1 ; c1ccc(-c2ccc(N3c4ccc(-c5ccccc5)cc4B4c5c3cc(-c3ccc(N(c6ccccc6)c6ccccc6)cc3)cc5N(c3ccc(-c5ccccc5)cc3)c3ncn(-c5ccccc5)c34)cc2)cc1]

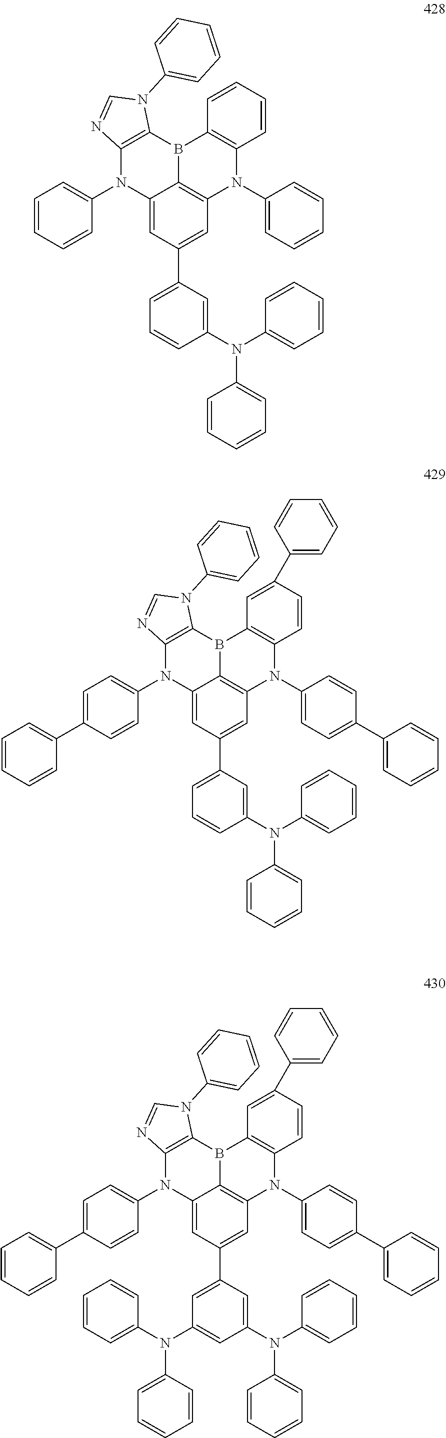 [SMILES: c1ccc(N(c2ccccc2)c2cccc(-c3cc4c5c(c3)N(c3ccccc3)c3ncn(-c6ccccc6)c3B5c3ccccc3N4c3ccccc3)c2)cc1 ; c1ccc(-c2ccc(N3c4ccc(-c5ccccc5)cc4B4c5c3cc(-c3cccc(N(c6ccccc6)c6ccccc6)c3)cc5N(c3ccc(-c5ccccc5)cc3)c3ncn(-c5ccccc5)c34)cc2)cc1 ; c1ccc(-c2ccc(N3c4ccc(-c5ccccc5)cc4B4c5c3cc(-c3cc(N(c6ccccc6)c6ccccc6)cc(N(c6ccccc6)c6ccccc6)c3)cc5N(c3ccc(-c5ccccc5)cc3)c3ncn(-c5ccccc5)c34)cc2)cc1]

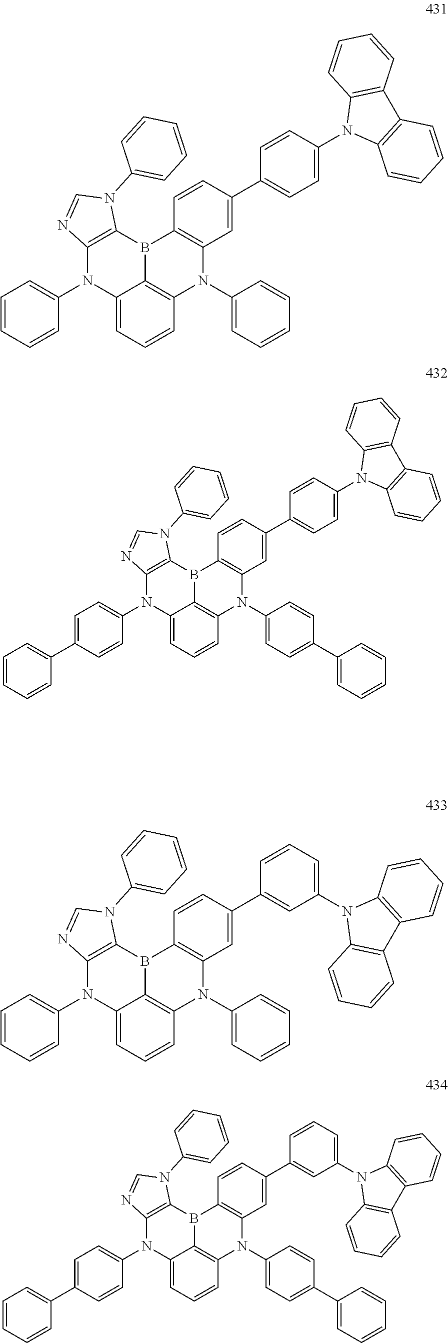 [SMILES: c1ccc(N2c3cc(-c4ccc(-n5c6ccccc6c6ccccc65)cc4)ccc3B3c4c2cccc4N(c2ccccc2)c2ncn(-c4ccccc4)c23)cc1 ; c1ccc(-c2ccc(N3c4cc(-c5ccc(-n6c7ccccc7c7ccccc76)cc5)ccc4B4c5c3cccc5N(c3ccc(-c5ccccc5)cc3)c3ncn(-c5ccccc5)c34)cc2)cc1 ; c1ccc(N2c3cc(-c4cccc(-n5c6ccccc6c6ccccc65)c4)ccc3B3c4c2cccc4N(c2ccccc2)c2ncn(-c4ccccc4)c23)cc1 ; c1ccc(-c2ccc(N3c4cc(-c5cccc(-n6c7ccccc7c7ccccc76)c5)ccc4B4c5c3cccc5N(c3ccc(-c5ccccc5)cc3)c3ncn(-c5ccccc5)c34)cc2)cc1]

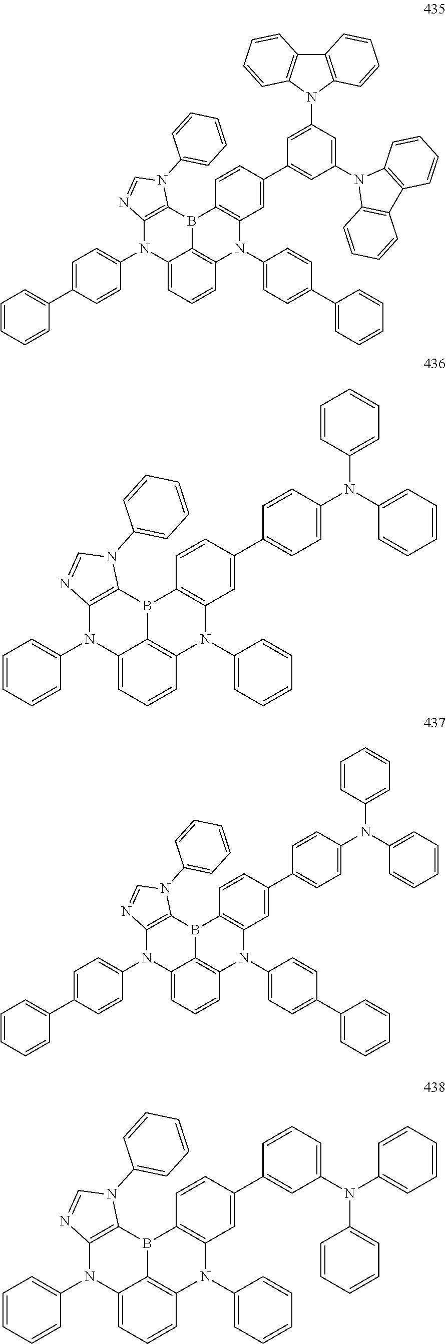 [SMILES: c1ccc(-c2ccc(N3c4cc(-c5cc(-n6c7ccccc7c7ccccc76)cc(-n6c7ccccc7c7ccccc76)c5)ccc4B4c5c3cccc5N(c3ccc(-c5ccccc5)cc3)c3ncn(-c5ccccc5)c34)cc2)cc1 ; c1ccc(N(c2ccccc2)c2ccc(-c3ccc4c(c3)N(c3ccccc3)c3cccc5c3B4c3c(ncn3-c3ccccc3)N5c3ccccc3)cc2)cc1 ; c1ccc(-c2ccc(N3c4cc(-c5ccc(N(c6ccccc6)c6ccccc6)cc5)ccc4B4c5c3cccc5N(c3ccc(-c5ccccc5)cc3)c3ncn(-c5ccccc5)c34)cc2)cc1 ; c1ccc(N(c2ccccc2)c2cccc(-c3ccc4c(c3)N(c3ccccc3)c3cccc5c3B4c3c(ncn3-c3ccccc3)N5c3ccccc3)c2)cc1]

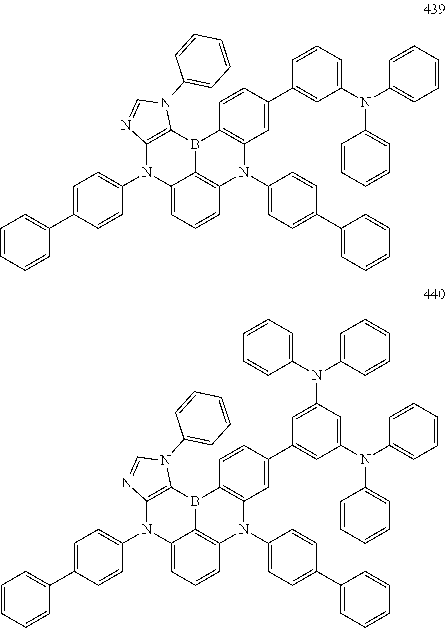 [SMILES: c1ccc(-c2ccc(N3c4cc(-c5cccc(N(c6ccccc6)c6ccccc6)c5)ccc4B4c5c3cccc5N(c3ccc(-c5ccccc5)cc3)c3ncn(-c5ccccc5)c34)cc2)cc1 ; c1ccc(-c2ccc(N3c4cc(-c5cc(N(c6ccccc6)c6ccccc6)cc(N(c6ccccc6)c6ccccc6)c5)ccc4B4c5c3cccc5N(c3ccc(-c5ccccc5)cc3)c3ncn(-c5ccccc5)c34)cc2)cc1]

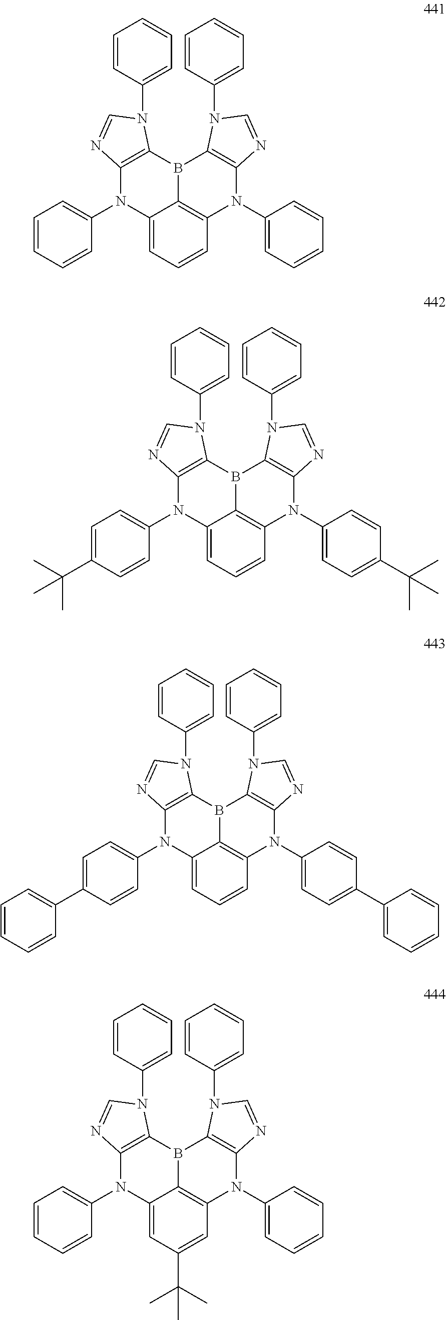 [SMILES: c1ccc(N2c3cccc4c3B(c3c2ncn3-c2ccccc2)c2c(ncn2-c2ccccc2)N4c2ccccc2)cc1 ; CC(C)(C)c1ccc(N2c3cccc4c3B(c3c2ncn3-c2ccccc2)c2c(ncn2-c2ccccc2)N4c2ccc(C(C)(C)C)cc2)cc1 ; c1ccc(-c2ccc(N3c4cccc5c4B(c4c3ncn4-c3ccccc3)c3c(ncn3-c3ccccc3)N5c3ccc(-c4ccccc4)cc3)cc2)cc1 ; CC(C)(C)c1cc2c3c(c1)N(c1ccccc1)c1ncn(-c4ccccc4)c1B3c1c(ncn1-c1ccccc1)N2c1ccccc1]

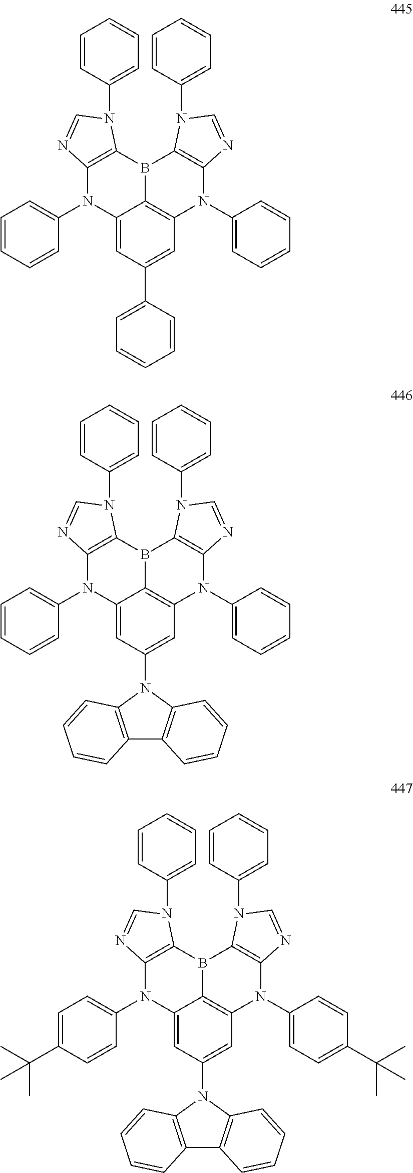 [SMILES: c1ccc(-c2cc3c4c(c2)N(c2ccccc2)c2ncn(-c5ccccc5)c2B4c2c(ncn2-c2ccccc2)N3c2ccccc2)cc1 ; c1ccc(N2c3cc(-n4c5ccccc5c5ccccc54)cc4c3B(c3c2ncn3-c2ccccc2)c2c(ncn2-c2ccccc2)N4c2ccccc2)cc1 ; CC(C)(C)c1ccc(N2c3cc(-n4c5ccccc5c5ccccc54)cc4c3B(c3c2ncn3-c2ccccc2)c2c(ncn2-c2ccccc2)N4c2ccc(C(C)(C)C)cc2)cc1]

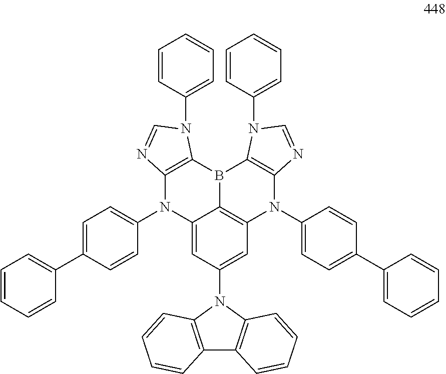 [SMILES: c1ccc(-c2ccc(N3c4cc(-n5c6ccccc6c6ccccc65)cc5c4B(c4c3ncn4-c3ccccc3)c3c(ncn3-c3ccccc3)N5c3ccc(-c4ccccc4)cc3)cc2)cc1]

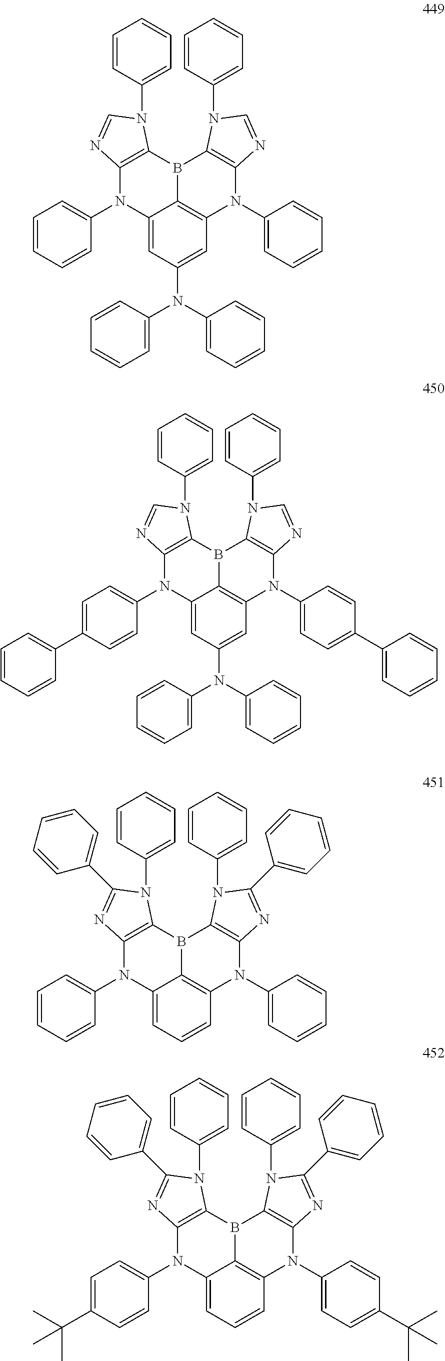

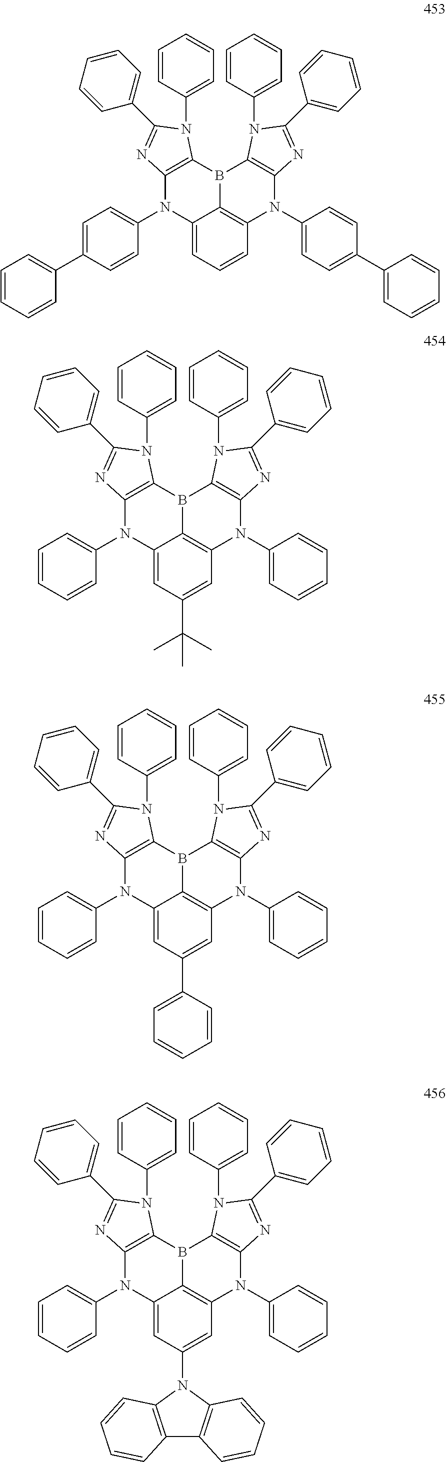 [SMILES: c1ccc(-c2ccc(N3c4cccc5c4B(c4c3nc(-c3ccccc3)n4-c3ccccc3)c3c(nc(-c4ccccc4)n3-c3ccccc3)N5c3ccc(-c4ccccc4)cc3)cc2)cc1 ; CC(C)(C)c1cc2c3c(c1)N(c1ccccc1)c1nc(-c4ccccc4)n(-c4ccccc4)c1B3c1c(nc(-c3ccccc3)n1-c1ccccc1)N2c1ccccc1 ; c1ccc(-c2cc3c4c(c2)N(c2ccccc2)c2nc(-c5ccccc5)n(-c5ccccc5)c2B4c2c(nc(-c4ccccc4)n2-c2ccccc2)N3c2ccccc2)cc1 ; c1ccc(-c2nc3c(n2-c2ccccc2)B2c4c(cc(-n5c6ccccc6c6ccccc65)cc4N(c4ccccc4)c4nc(-c5ccccc5)n(-c5ccccc5)c42)N3c2ccccc2)cc1]

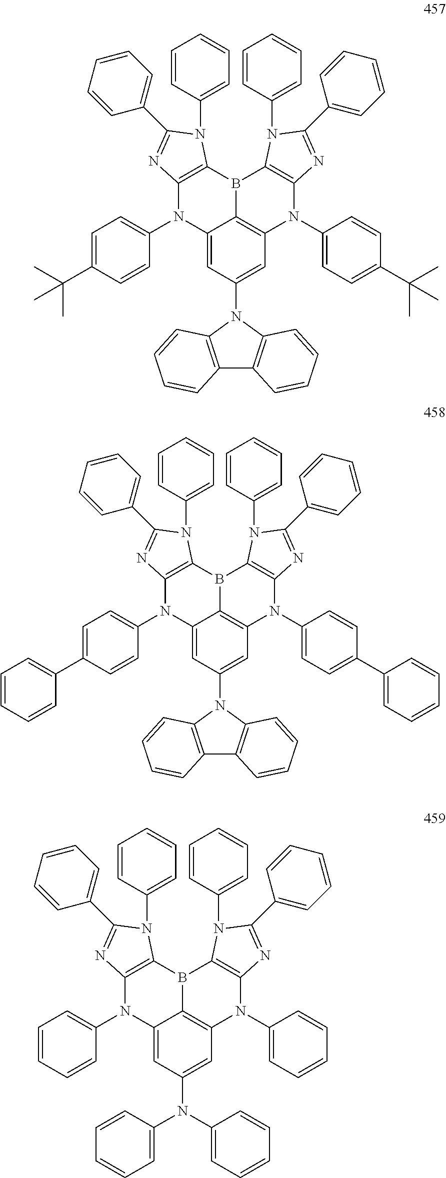

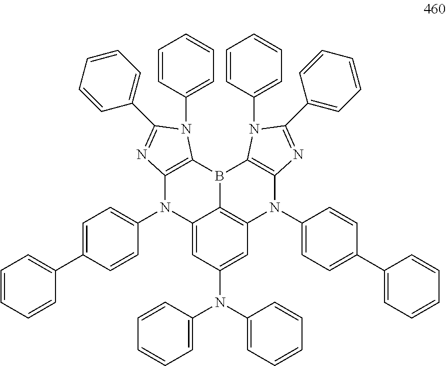 [SMILES: c1ccc(-c2ccc(N3c4cc(N(c5ccccc5)c5ccccc5)cc5c4B(c4c3nc(-c3ccccc3)n4-c3ccccc3)c3c(nc(-c4ccccc4)n3-c3ccccc3)N5c3ccc(-c4ccccc4)cc3)cc2)cc1]

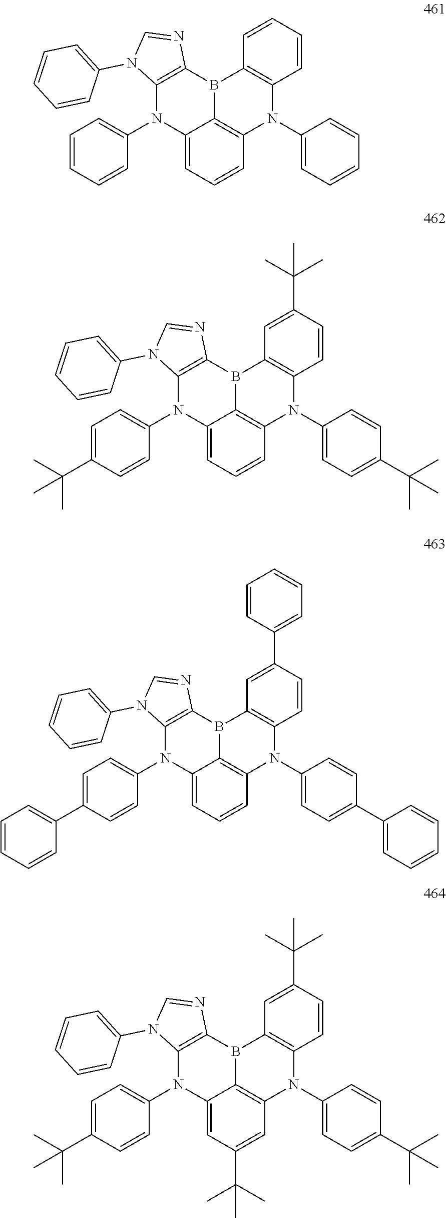

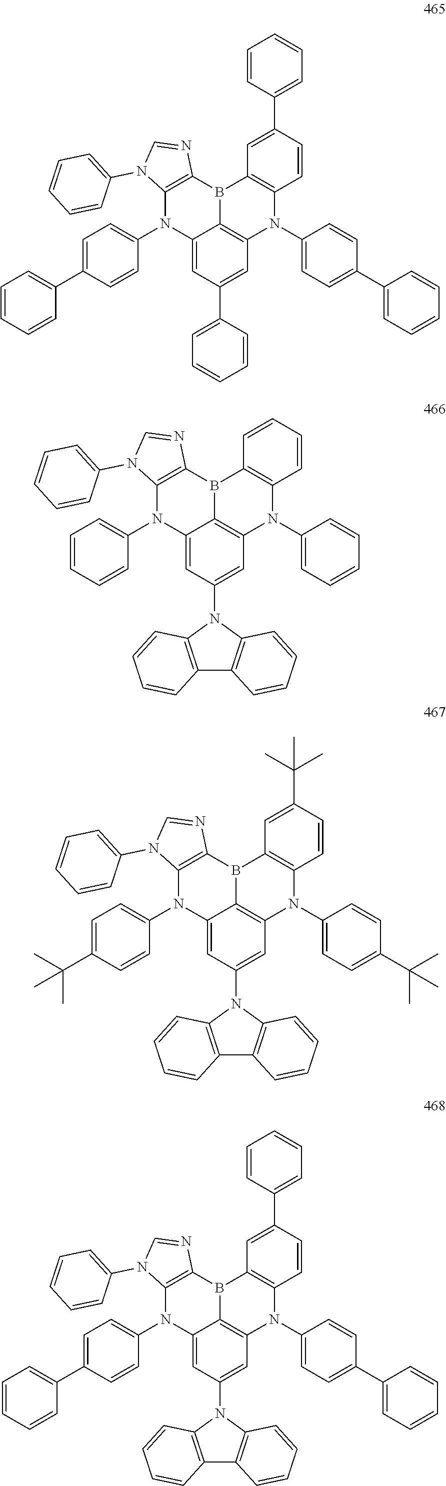 [SMILES: c1ccc(-c2ccc(N3c4ccc(-c5ccccc5)cc4B4c5ncn(-c6ccccc6)c5N(c5ccc(-c6ccccc6)cc5)c5cc(-c6ccccc6)cc3c54)cc2)cc1 ; c1ccc(N2c3ccccc3B3c4ncn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3B3c4ncn(-c5ccccc5)c4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4B4c5ncn(-c6ccccc6)c5N(c5ccc(-c6ccccc6)cc5)c5cc(-n6c7ccccc7c7ccccc76)cc3c54)cc2)cc1]

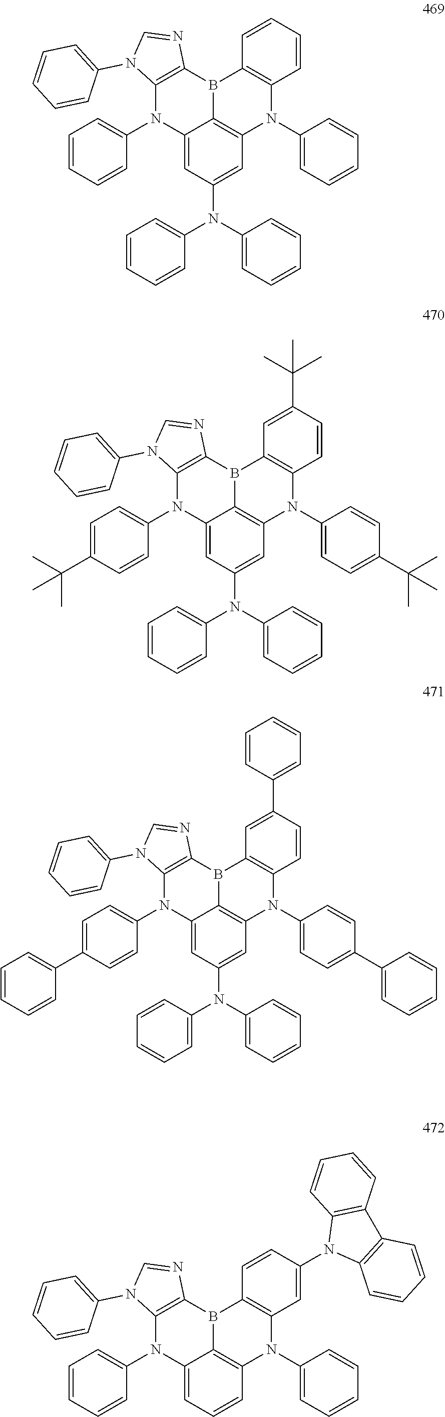 [SMILES: c1ccc(N(c2ccccc2)c2cc3c4c(c2)N(c2ccccc2)c2c(ncn2-c2ccccc2)B4c2ccccc2N3c2ccccc2)cc1 ; CC(C)(C)c1ccc(N2c3ccc(C(C)(C)C)cc3B3c4ncn(-c5ccccc5)c4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5ncn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4ncn(-c5ccccc5)c4N(c4ccccc4)c4cccc2c43)cc1]

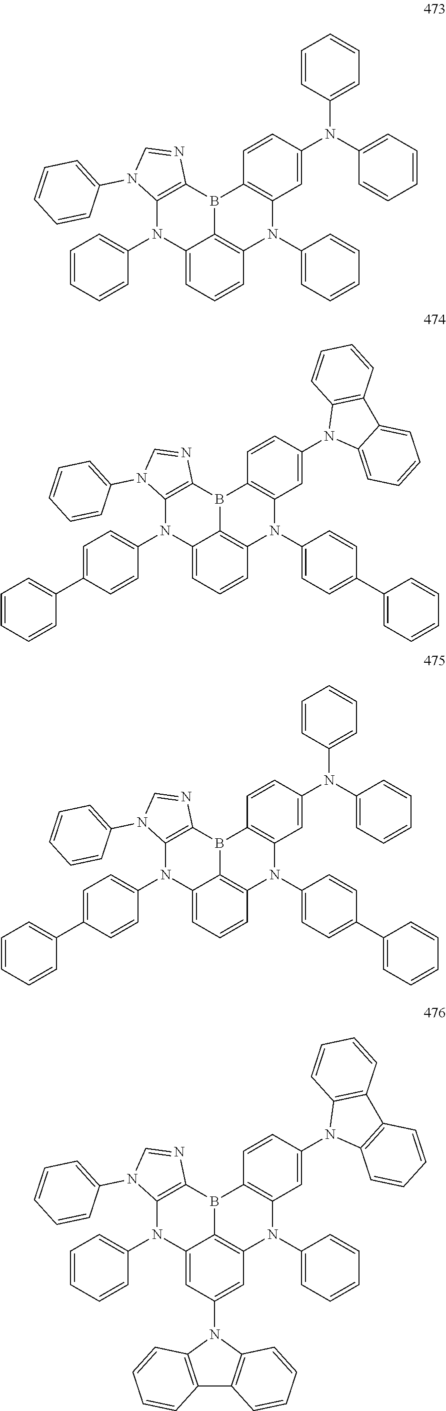 [SMILES: c1ccc(N(c2ccccc2)c2ccc3c(c2)N(c2ccccc2)c2cccc4c2B3c2ncn(-c3ccccc3)c2N4c2ccccc2)cc1 ; c1ccc(-c2ccc(N3c4cc(-n5c6ccccc6c6ccccc65)ccc4B4c5ncn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5ncn(-c6ccccc6)c5N(c5ccc(-c6ccccc6)cc5)c5cccc3c54)cc2)cc1 ; c1ccc(N2c3cc(-n4c5ccccc5c5ccccc54)ccc3B3c4ncn(-c5ccccc5)c4N(c4ccccc4)c4cc(-n5c6ccccc6c6ccccc65)cc2c43)cc1]

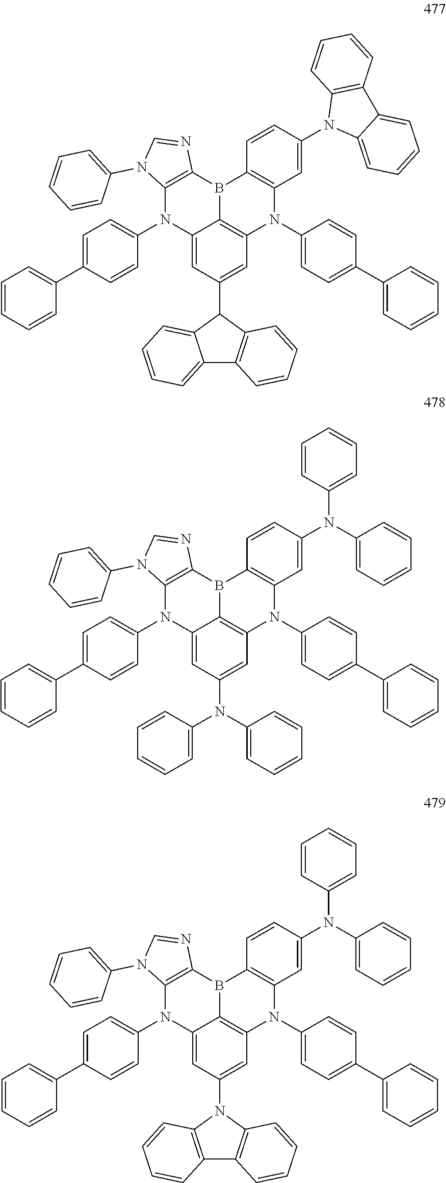 [SMILES: c1ccc(-c2ccc(N3c4cc(-n5c6ccccc6c6ccccc65)ccc4B4c5ncn(-c6ccccc6)c5N(c5ccc(-c6ccccc6)cc5)c5cc(C6c7ccccc7-c7ccccc76)cc3c54)cc2)cc1 ; c1ccc(-c2ccc(N3c4cc(N(c5ccccc5)c5ccccc5)ccc4B4c5ncn(-c6ccccc6)c5N(c5ccc(-c6ccccc6)cc5)c5cc(N(c6ccccc6)c6ccccc6)cc3c54)cc2)cc1 ; c1ccc(-c2ccc(N3c4cc(N(c5ccccc5)c5ccccc5)ccc4B4c5ncn(-c6ccccc6)c5N(c5ccc(-c6ccccc6)cc5)c5cc(-n6c7ccccc7c7ccccc76)cc3c54)cc2)cc1]

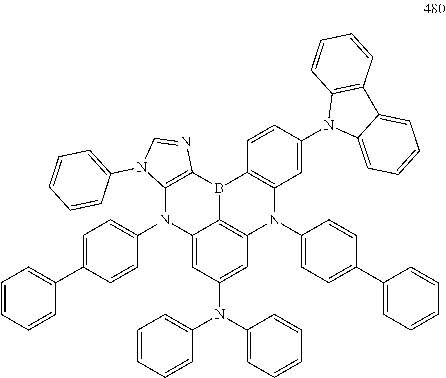 [SMILES: c1ccc(-c2ccc(N3c4cc(-n5c6ccccc6c6ccccc65)ccc4B4c5ncn(-c6ccccc6)c5N(c5ccc(-c6ccccc6)cc5)c5cc(N(c6ccccc6)c6ccccc6)cc3c54)cc2)cc1]

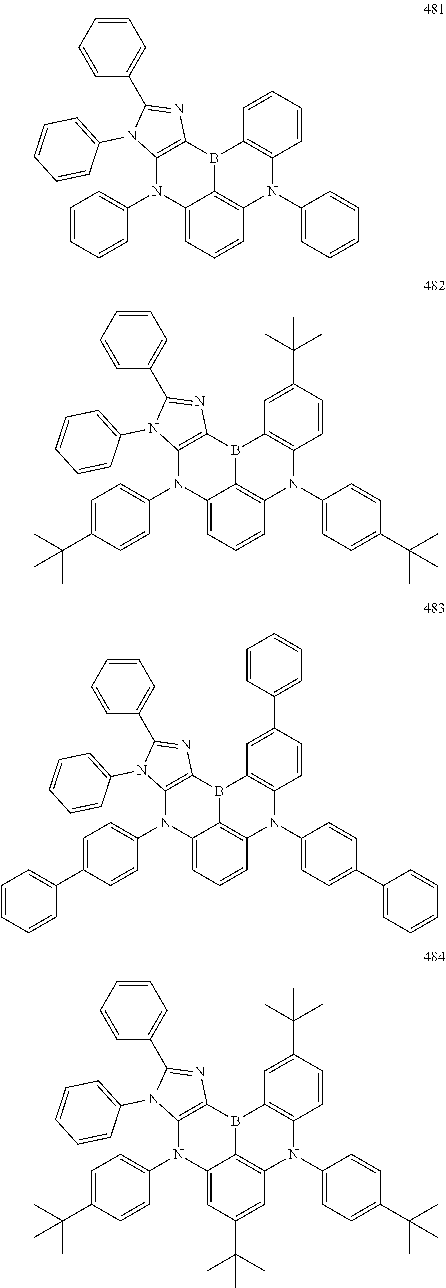

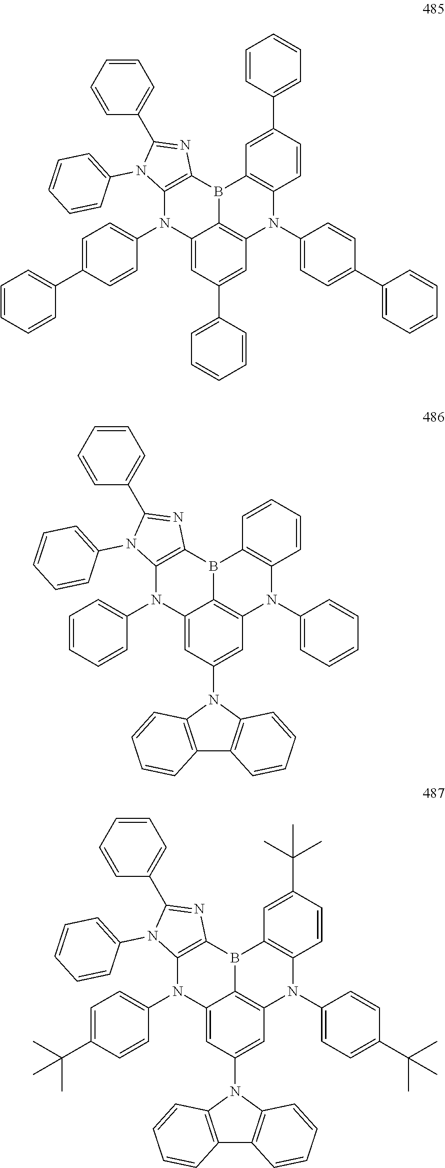 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(-c6ccccc6)cc3c54)cc2)cc1 ; c1ccc(-c2nc3c(n2-c2ccccc2)N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4N(c4ccc(C(C)(C)C)cc4)c4cc(-n5c6ccccc6c6ccccc65)cc2c43)cc1]

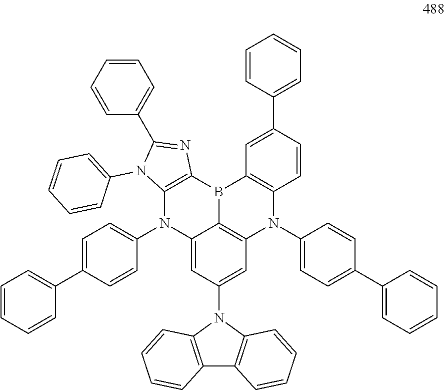 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7ccccc76)cc3c54)cc2)cc1]

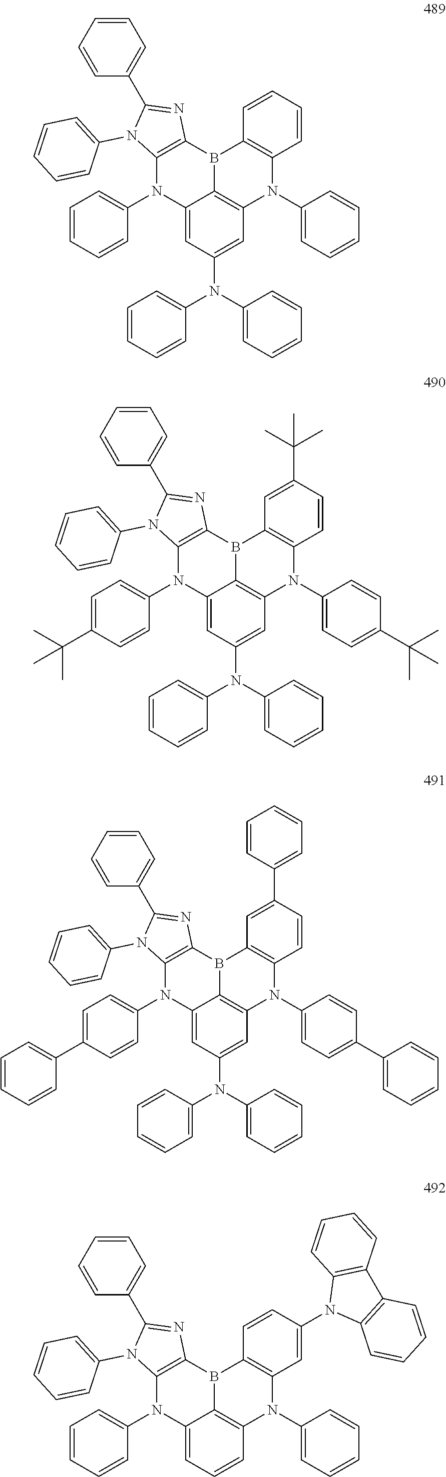 [SMILES: c1ccc(-c2nc3c(n2-c2ccccc2)N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(N(c6ccccc6)c6ccccc6)cc3c54)cc2)cc1 ; c1ccc(-c2nc3c(n2-c2ccccc2)N(c2ccccc2)c2cccc4c2B3c2ccc(-n3c5ccccc5c5ccccc53)cc2N4c2ccccc2)cc1]

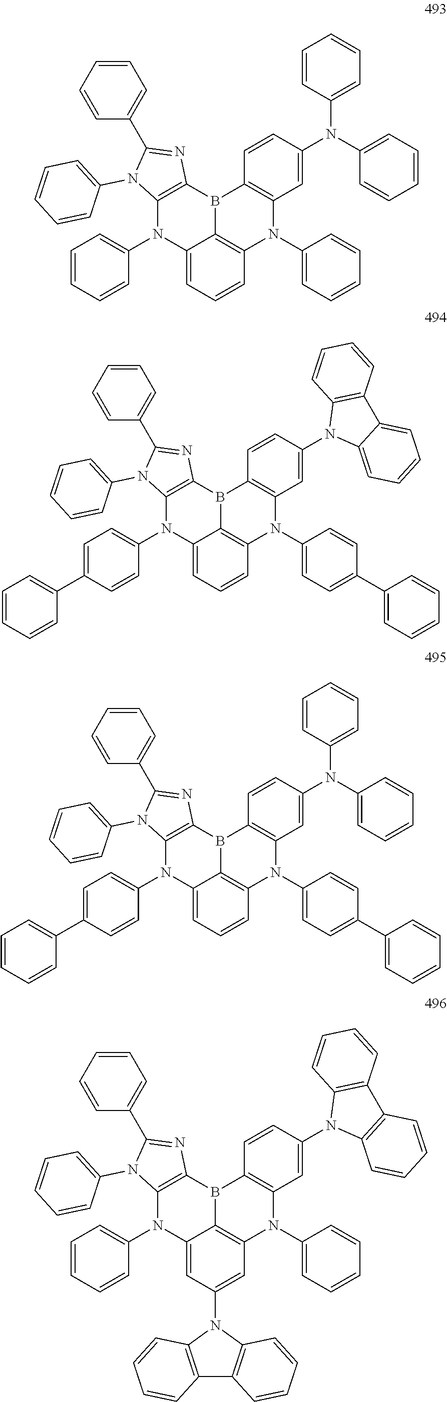 [SMILES: c1ccc(-c2nc3c(n2-c2ccccc2)N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5nc(-c6ccccc6)n(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cccc3c54)cc2)cc1 ; c1ccc(-c2nc3c(n2-c2ccccc2)N(c2ccccc2)c2cc(-n4c5ccccc5c5ccccc54)cc4c2B3c2ccc(-n3c5ccccc5c5ccccc53)cc2N4c2ccccc2)cc1]

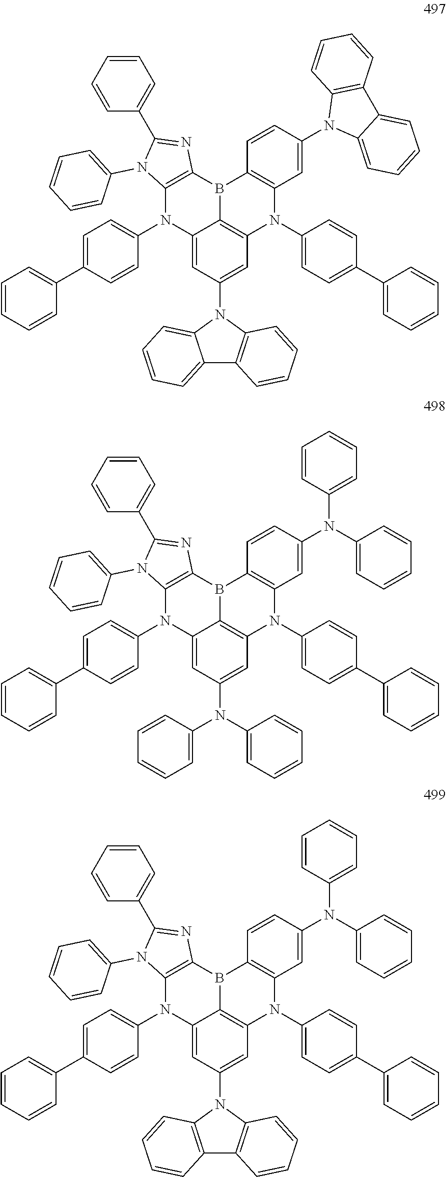 [SMILES: c1ccc(-c2ccc(N3c4cc(-n5c6ccccc6c6ccccc65)ccc4B4c5nc(-c6ccccc6)n(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cc(N(c6ccccc6)c6ccccc6)cc3c54)cc2)cc1 ; c1ccc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cc(-n6c7ccccc7c7ccccc76)cc3c54)cc2)cc1]

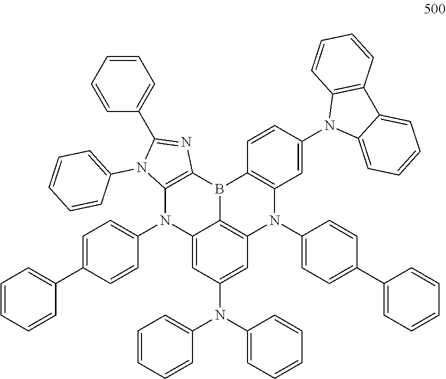 [SMILES: c1ccc(-c2ccc(N3c4cc(-n5c6ccccc6c6ccccc65)ccc4B4c5nc(-c6ccccc6)n(-c6ccccc6)c5N(c5ccc(-c6ccccc6)cc5)c5cc(N(c6ccccc6)c6ccccc6)cc3c54)cc2)cc1]

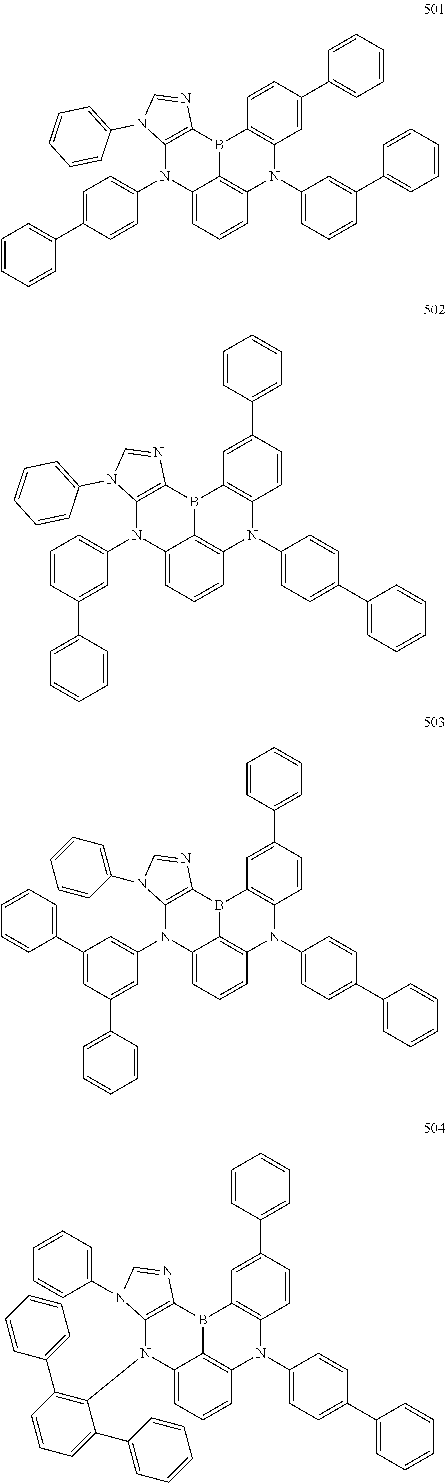 [SMILES: c1ccc(-c2ccc(N3c4cccc5c4B(c4ccc(-c6ccccc6)cc4N5c4cccc(-c5ccccc5)c4)c4ncn(-c5ccccc5)c43)cc2)cc1 ; c1ccc(-c2ccc(N3c4ccc(-c5ccccc5)cc4B4c5ncn(-c6ccccc6)c5N(c5cccc(-c6ccccc6)c5)c5cccc3c54)cc2)cc1 ; c1ccc(-c2ccc(N3c4ccc(-c5ccccc5)cc4B4c5ncn(-c6ccccc6)c5N(c5cc(-c6ccccc6)cc(-c6ccccc6)c5)c5cccc3c54)cc2)cc1 ; c1ccc(-c2ccc(N3c4ccc(-c5ccccc5)cc4B4c5ncn(-c6ccccc6)c5N(c5c(-c6ccccc6)cccc5-c5ccccc5)c5cccc3c54)cc2)cc1]

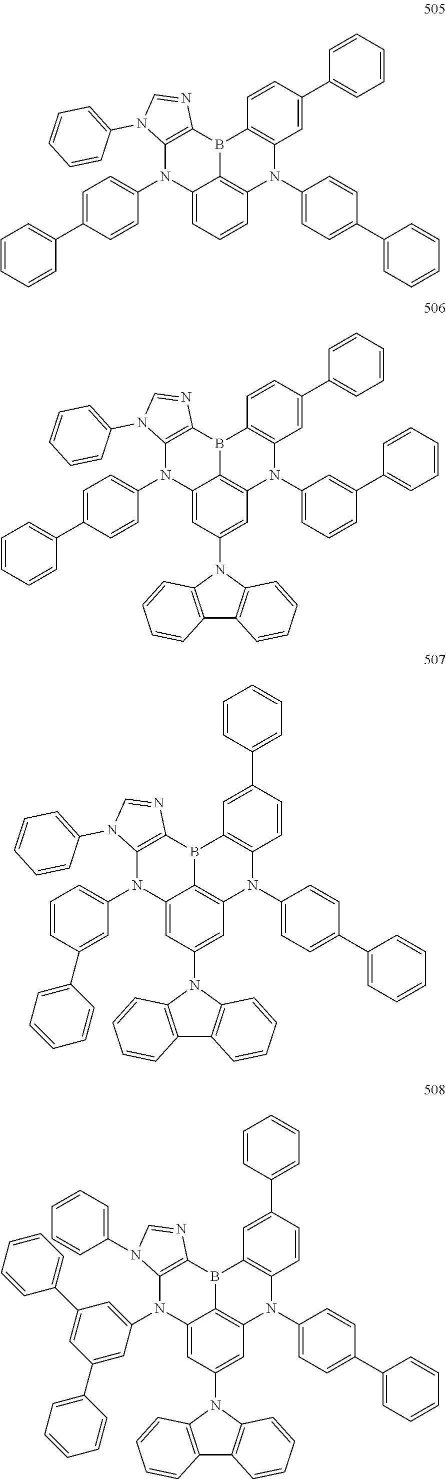

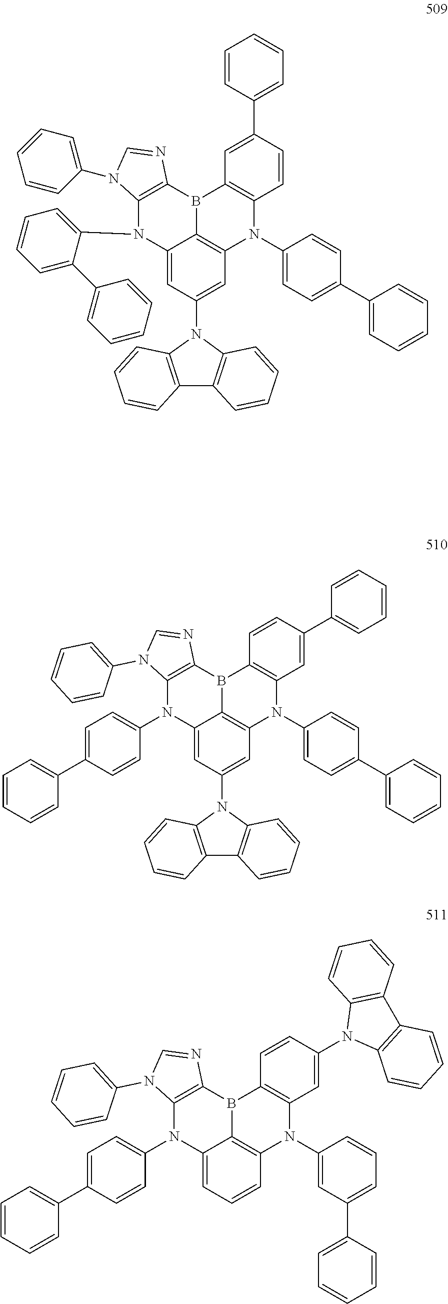 [SMILES: c1ccc(-c2ccc(N3c4ccc(-c5ccccc5)cc4B4c5ncn(-c6ccccc6)c5N(c5ccccc5-c5ccccc5)c5cc(-n6c7ccccc7c7ccccc76)cc3c54)cc2)cc1 ; c1ccc(-c2ccc(N3c4cc(-c5ccccc5)ccc4B4c5ncn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cccc5c4B(c4ccc(-n6c7ccccc7c7ccccc76)cc4N5c4cccc(-c5ccccc5)c4)c4ncn(-c5ccccc5)c43)cc2)cc1]

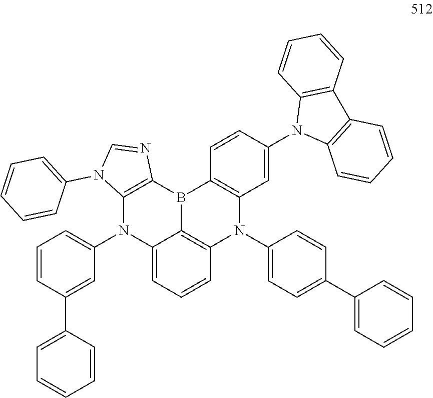 [SMILES: c1ccc(-c2ccc(N3c4cc(-n5c6ccccc6c6ccccc65)ccc4B4c5ncn(-c6ccccc6)c5N(c5cccc(-c6ccccc6)c5)c5cccc3c54)cc2)cc1]

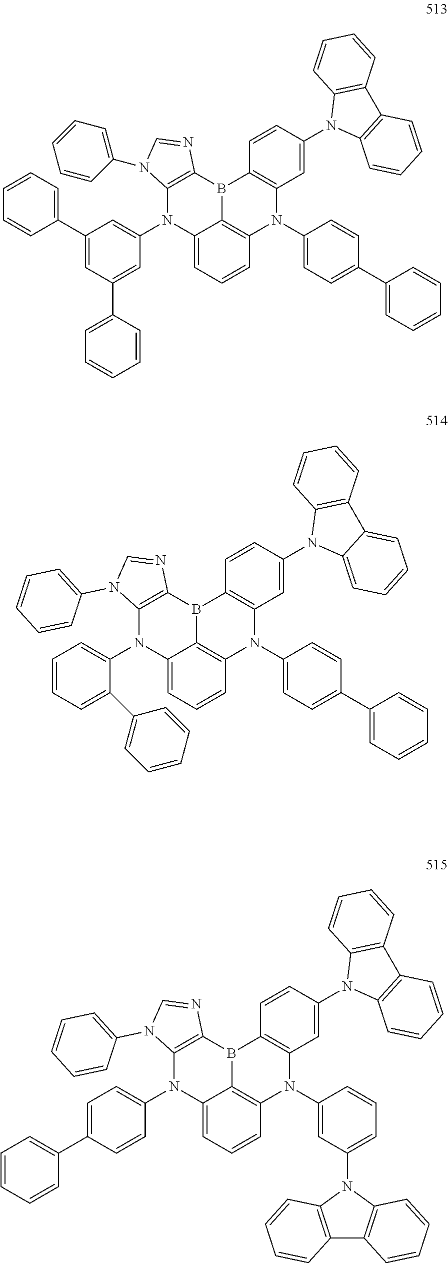 [SMILES: c1ccc(-c2ccc(N3c4cc(-n5c6ccccc6c6ccccc65)ccc4B4c5ncn(-c6ccccc6)c5N(c5cc(-c6ccccc6)cc(-c6ccccc6)c5)c5cccc3c54)cc2)cc1 ; c1ccc(-c2ccc(N3c4cc(-n5c6ccccc6c6ccccc65)ccc4B4c5ncn(-c6ccccc6)c5N(c5ccccc5-c5ccccc5)c5cccc3c54)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4ncn(-c5ccccc5)c43)cc2)cc1]

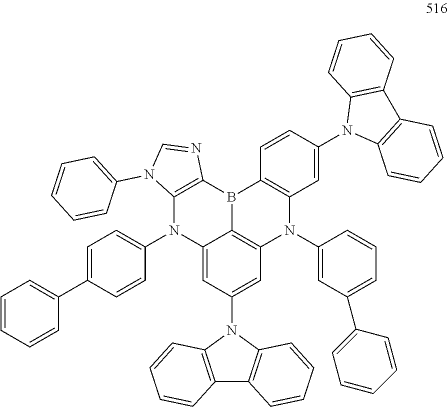 [SMILES: c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4ncn(-c5ccccc5)c43)cc2)cc1]

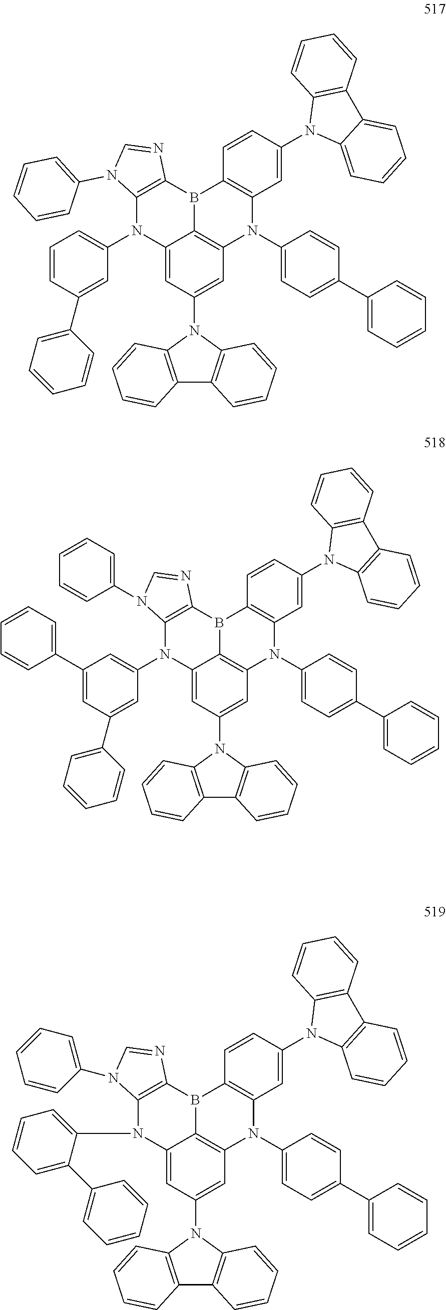 [SMILES: c1ccc(-c2ccc(N3c4cc(-n5c6ccccc6c6ccccc65)ccc4B4c5ncn(-c6ccccc6)c5N(c5cccc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ncn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ncn(-c6ccccc6)c5N(c5ccccc5-c5ccccc5)c5cc(-n6c7ccccc7c7ccccc76)cc3c54)cc2)cc1]

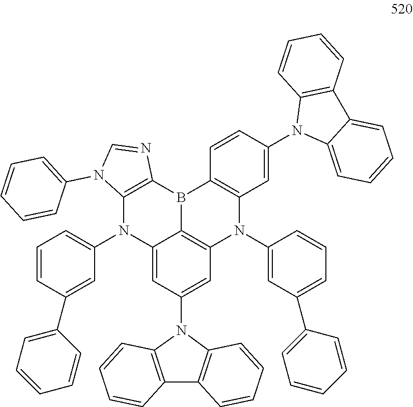 [SMILES: c1ccc(-c2cccc(N3c4cc(-n5c6ccccc6c6ccccc65)ccc4B4c5ncn(-c6ccccc6)c5N(c5cccc(-c6ccccc6)c5)c5cc(-n6c7ccccc7c7ccccc76)cc3c54)c2)cc1]

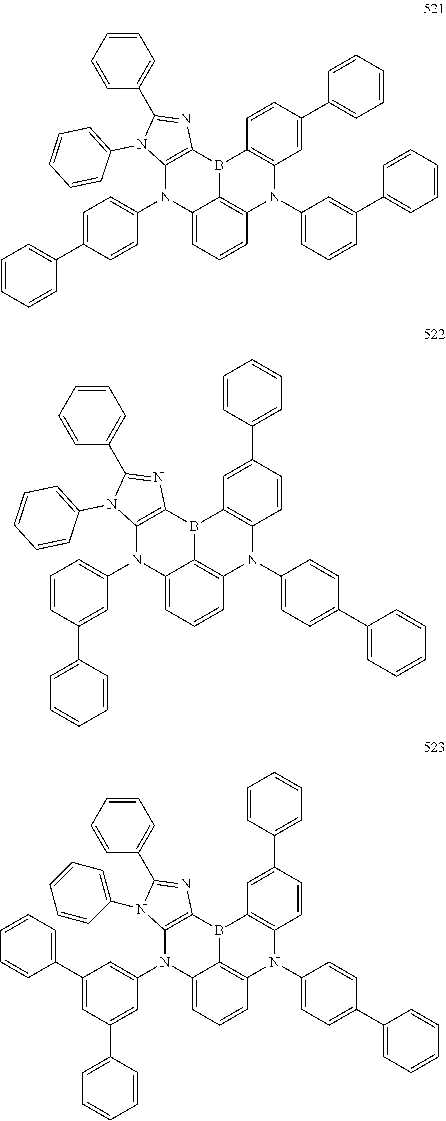 [SMILES: c1ccc(-c2ccc(N3c4cccc5c4B(c4ccc(-c6ccccc6)cc4N5c4cccc(-c5ccccc5)c4)c4nc(-c5ccccc5)n(-c5ccccc5)c43)cc2)cc1 ; c1ccc(-c2ccc(N3c4ccc(-c5ccccc5)cc4B4c5nc(-c6ccccc6)n(-c6ccccc6)c5N(c5cccc(-c6ccccc6)c5)c5cccc3c54)cc2)cc1 ; c1ccc(-c2ccc(N3c4ccc(-c5ccccc5)cc4B4c5nc(-c6ccccc6)n(-c6ccccc6)c5N(c5cc(-c6ccccc6)cc(-c6ccccc6)c5)c5cccc3c54)cc2)cc1]

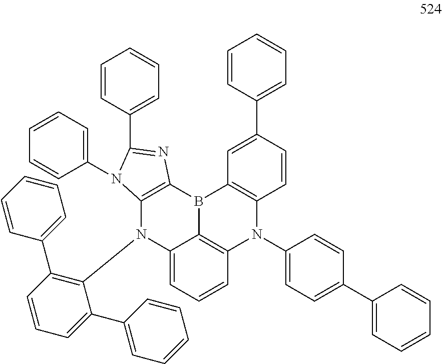 [SMILES: c1ccc(-c2ccc(N3c4ccc(-c5ccccc5)cc4B4c5nc(-c6ccccc6)n(-c6ccccc6)c5N(c5c(-c6ccccc6)cccc5-c5ccccc5)c5cccc3c54)cc2)cc1]

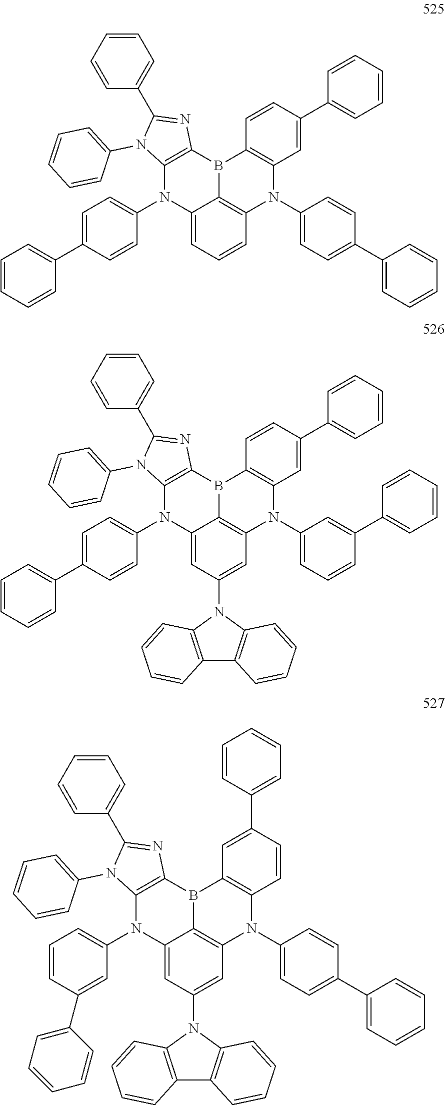 [SMILES: c1ccc(-c2ccc(N3c4cc(-c5ccccc5)ccc4B4c5nc(-c6ccccc6)n(-c6ccccc6)c5N(c5ccc(-c6ccccc6)cc5)c5cccc3c54)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4nc(-c5ccccc5)n(-c5ccccc5)c43)cc2)cc1 ; c1ccc(-c2ccc(N3c4ccc(-c5ccccc5)cc4B4c5nc(-c6ccccc6)n(-c6ccccc6)c5N(c5cccc(-c6ccccc6)c5)c5cc(-n6c7ccccc7c7ccccc76)cc3c54)cc2)cc1]

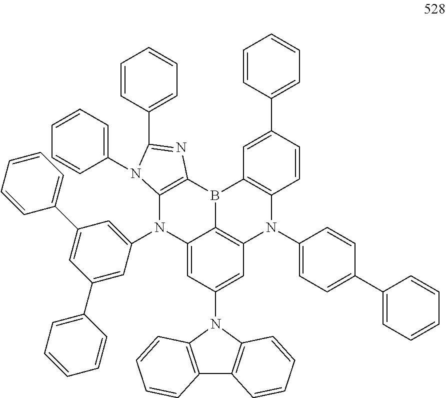 [SMILES: c1ccc(-c2ccc(N3c4ccc(-c5ccccc5)cc4B4c5nc(-c6ccccc6)n(-c6ccccc6)c5N(c5cc(-c6ccccc6)cc(-c6ccccc6)c5)c5cc(-n6c7ccccc7c7ccccc76)cc3c54)cc2)cc1]

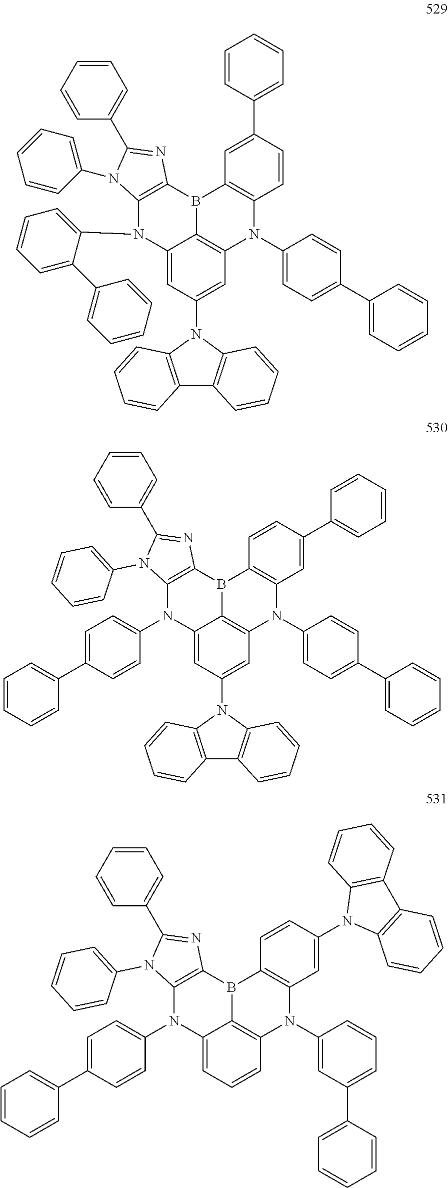

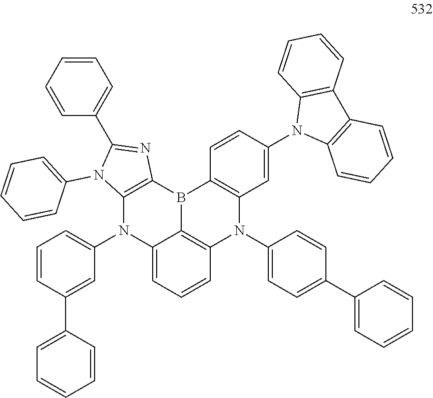 [SMILES: c1ccc(-c2ccc(N3c4cc(-n5c6ccccc6c6ccccc65)ccc4B4c5nc(-c6ccccc6)n(-c6ccccc6)c5N(c5cccc(-c6ccccc6)c5)c5cccc3c54)cc2)cc1]

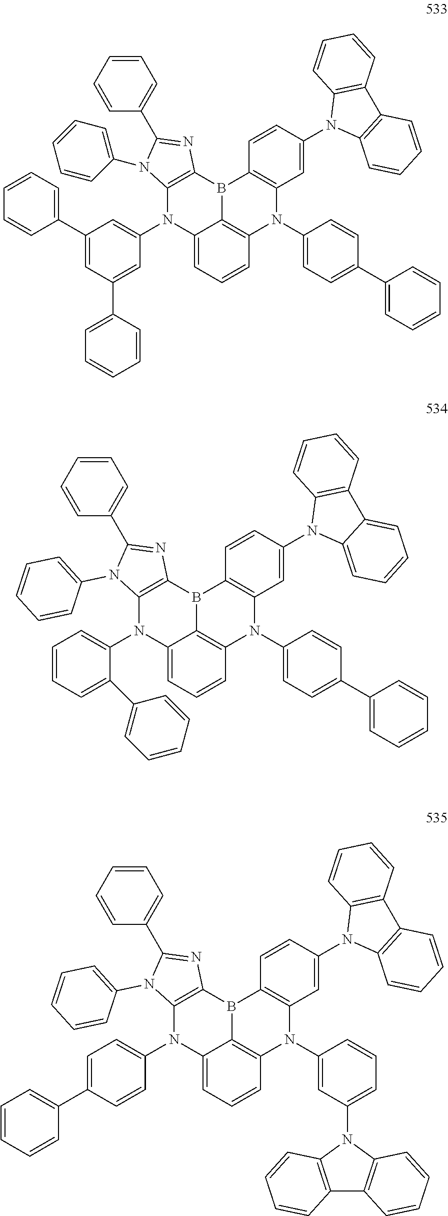 [SMILES: c1ccc(-c2ccc(N3c4cc(-n5c6ccccc6c6ccccc65)ccc4B4c5nc(-c6ccccc6)n(-c6ccccc6)c5N(c5cc(-c6ccccc6)cc(-c6ccccc6)c5)c5cccc3c54)cc2)cc1 ; c1ccc(-c2ccc(N3c4cc(-n5c6ccccc6c6ccccc65)ccc4B4c5nc(-c6ccccc6)n(-c6ccccc6)c5N(c5ccccc5-c5ccccc5)c5cccc3c54)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4nc(-c5ccccc5)n(-c5ccccc5)c43)cc2)cc1]

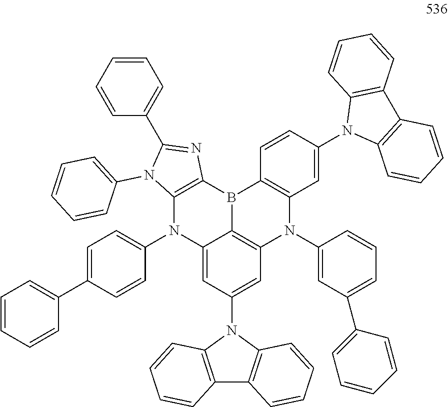 [SMILES: c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4nc(-c5ccccc5)n(-c5ccccc5)c43)cc2)cc1]

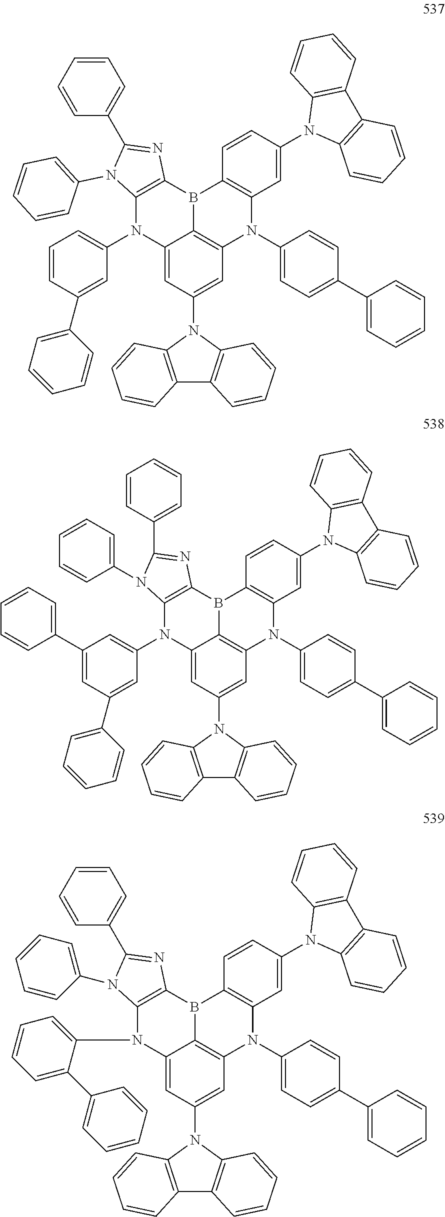 [SMILES: c1ccc(-c2ccc(N3c4cc(-n5c6ccccc6c6ccccc65)ccc4B4c5nc(-c6ccccc6)n(-c6ccccc6)c5N(c5cccc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nc(-c6ccccc6)n(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nc(-c6ccccc6)n(-c6ccccc6)c5N(c5ccccc5-c5ccccc5)c5cc(-n6c7ccccc7c7ccccc76)cc3c54)cc2)cc1]

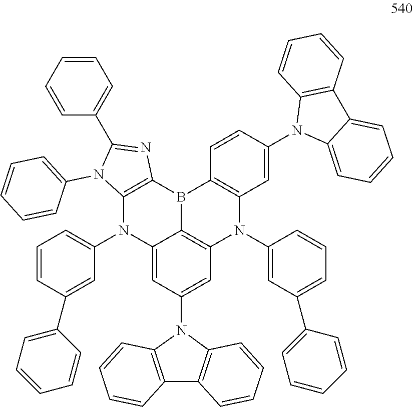 [SMILES: c1ccc(-c2cccc(N3c4cc(-n5c6ccccc6c6ccccc65)ccc4B4c5nc(-c6ccccc6)n(-c6ccccc6)c5N(c5cccc(-c6ccccc6)c5)c5cc(-n6c7ccccc7c7ccccc76)cc3c54)c2)cc1]

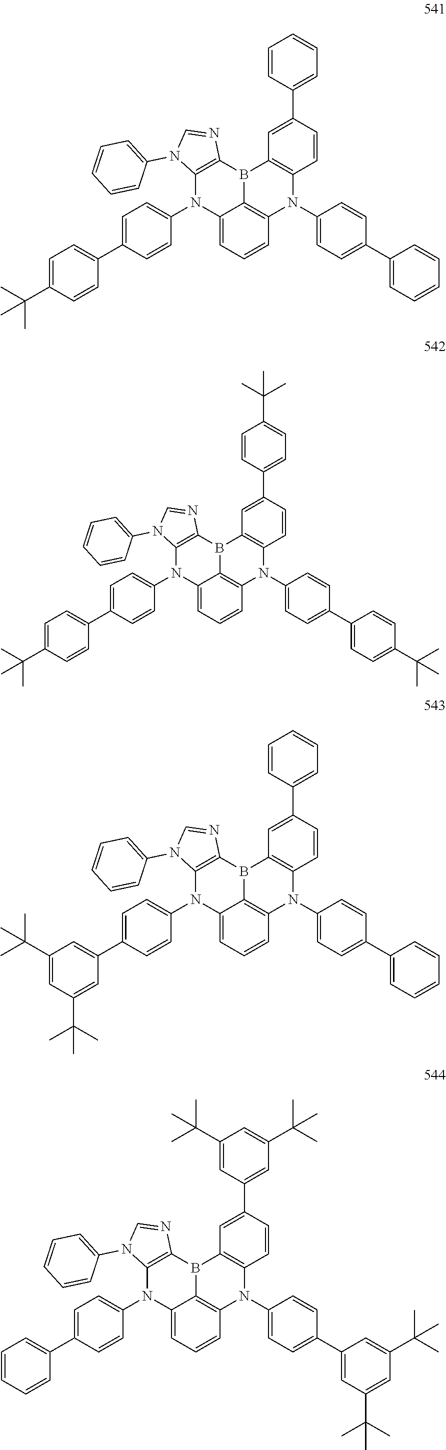

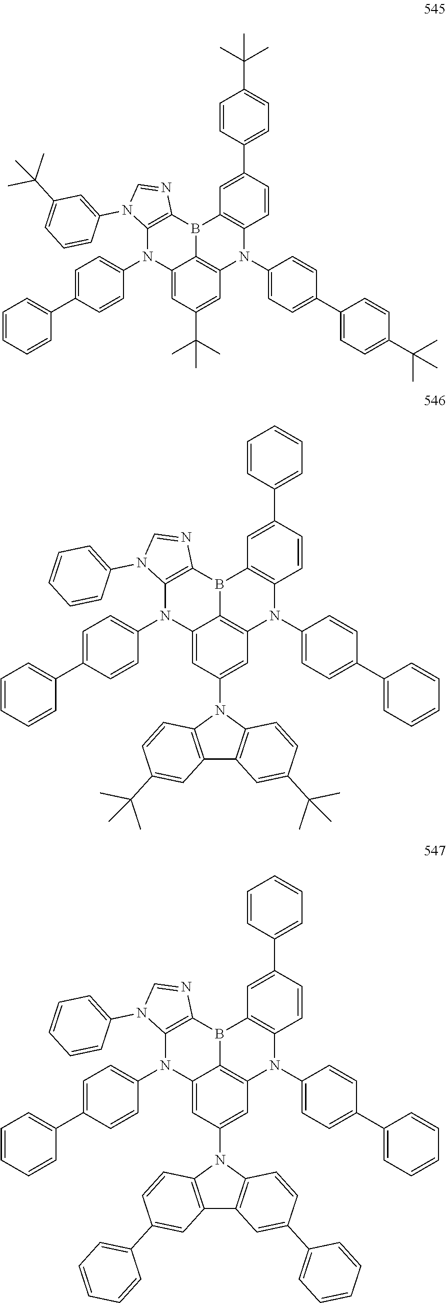 [SMILES: CC(C)(C)c1ccc(-c2ccc(N3c4ccc(-c5ccc(C(C)(C)C)cc5)cc4B4c5ncn(-c6cccc(C(C)(C)C)c6)c5N(c5ccc(-c6ccccc6)cc5)c5cc(C(C)(C)C)cc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(ncn1-c1ccccc1)B3c1cc(-c3ccccc3)ccc1N2c1ccc(-c2ccccc2)cc1 ; c1ccc(-c2ccc(N3c4ccc(-c5ccccc5)cc4B4c5ncn(-c6ccccc6)c5N(c5ccc(-c6ccccc6)cc5)c5cc(-n6c7ccc(-c8ccccc8)cc7c7cc(-c8ccccc8)ccc76)cc3c54)cc2)cc1]

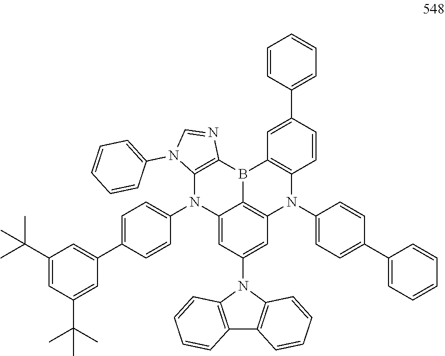 [SMILES: CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4ncn(-c5ccccc5)c43)cc2)cc(C(C)(C)C)c1]

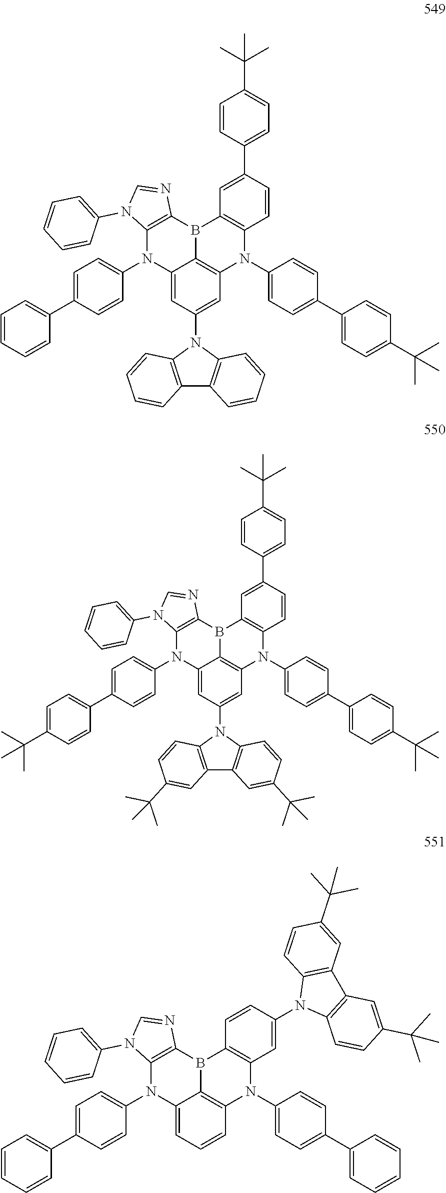

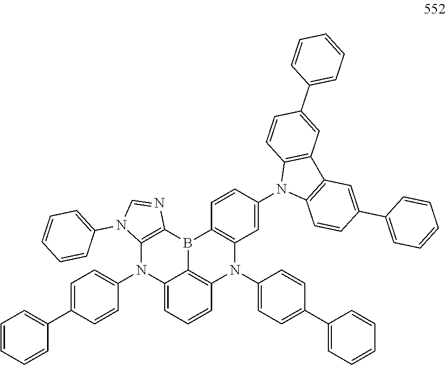 [SMILES: c1ccc(-c2ccc(N3c4cc(-n5c6ccc(-c7ccccc7)cc6c6cc(-c7ccccc7)ccc65)ccc4B4c5ncn(-c6ccccc6)c5N(c5ccc(-c6ccccc6)cc5)c5cccc3c54)cc2)cc1]

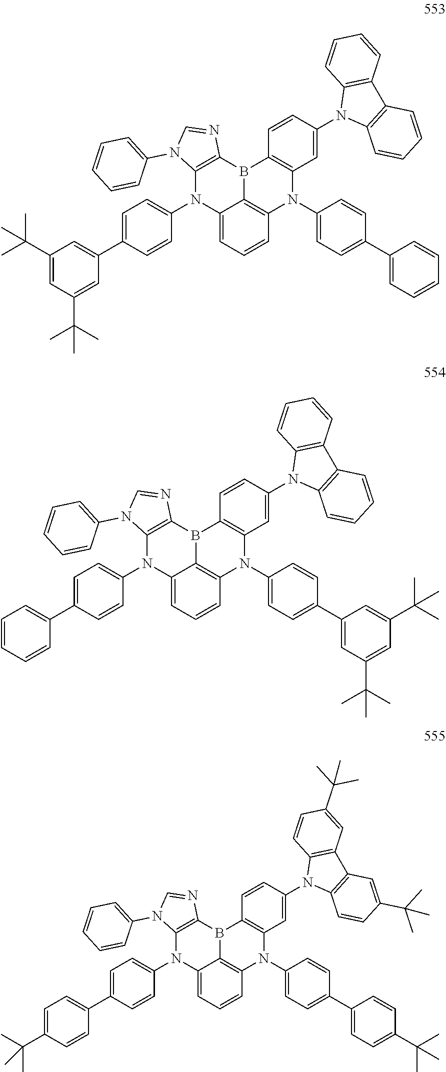

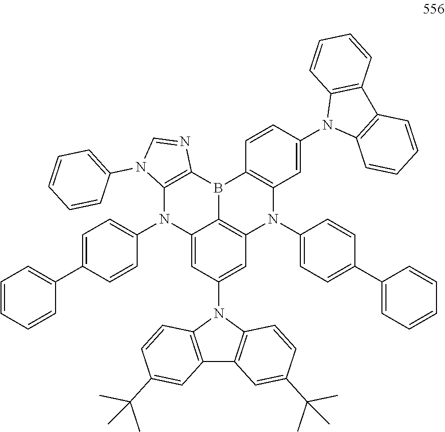 [SMILES: CC(C)(C)c1ccc2c(c1)c1cc(C(C)(C)C)ccc1n2-c1cc2c3c(c1)N(c1ccc(-c4ccccc4)cc1)c1c(ncn1-c1ccccc1)B3c1ccc(-n3c4ccccc4c4ccccc43)cc1N2c1ccc(-c2ccccc2)cc1]

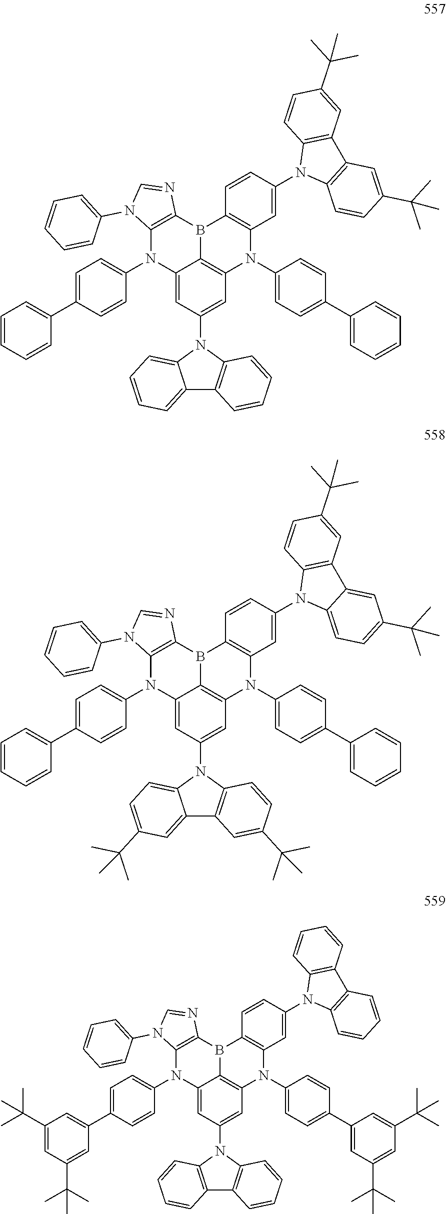

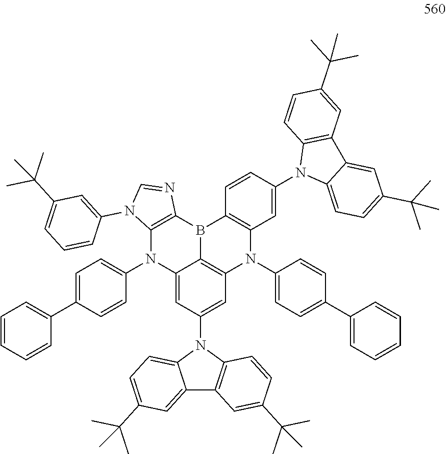 [SMILES: CC(C)(C)c1cccc(-n2cnc3c2N(c2ccc(-c4ccccc4)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccccc3)cc2)c1]

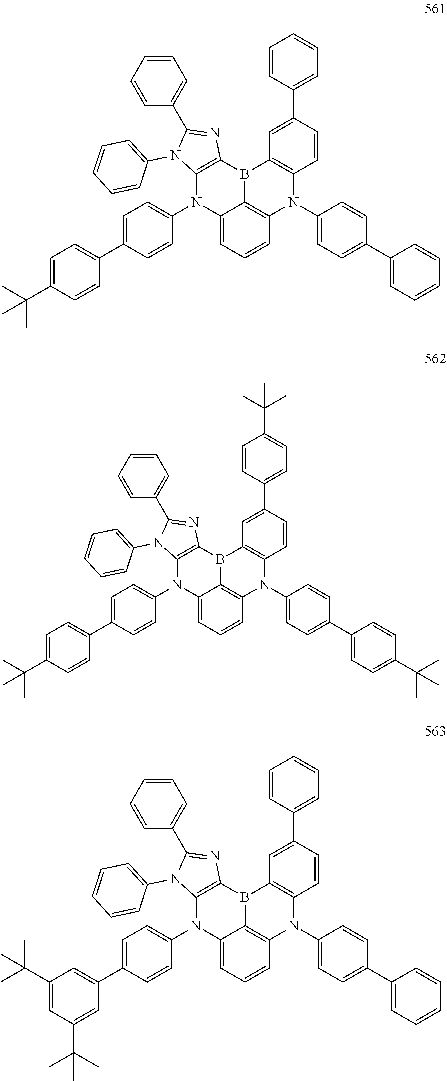

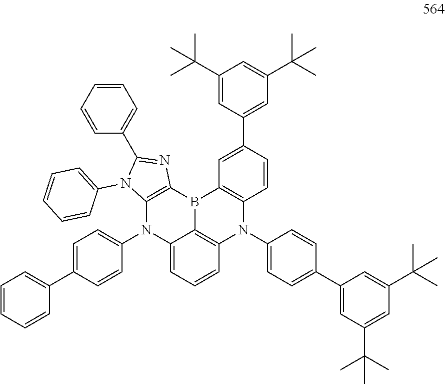 [SMILES: CC(C)(C)c1cc(-c2ccc(N3c4ccc(-c5cc(C(C)(C)C)cc(C(C)(C)C)c5)cc4B4c5nc(-c6ccccc6)n(-c6ccccc6)c5N(c5ccc(-c6ccccc6)cc5)c5cccc3c54)cc2)cc(C(C)(C)C)c1]

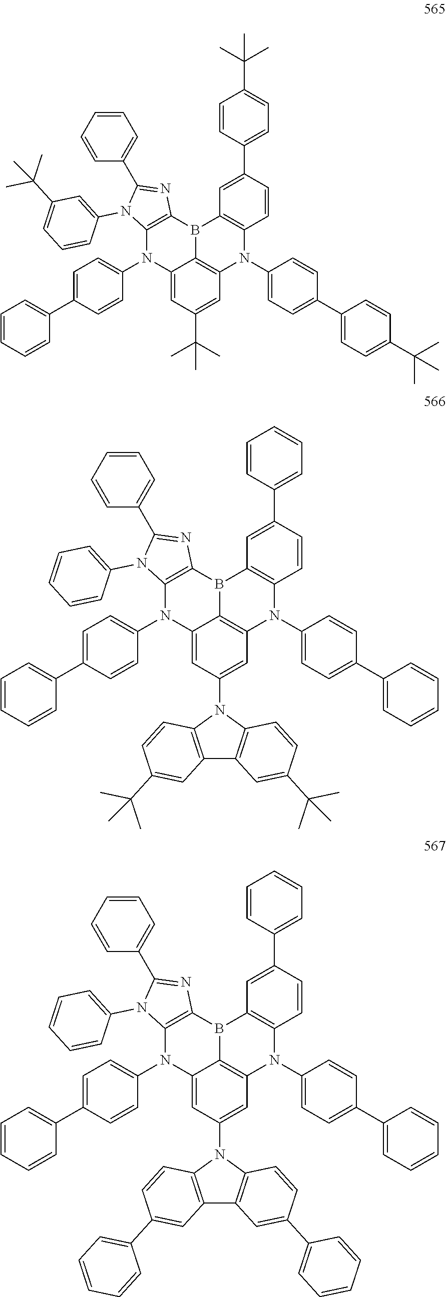 [SMILES: CC(C)(C)c1ccc(-c2ccc(N3c4ccc(-c5ccc(C(C)(C)C)cc5)cc4B4c5nc(-c6ccccc6)n(-c6cccc(C(C)(C)C)c6)c5N(c5ccc(-c6ccccc6)cc5)c5cc(C(C)(C)C)cc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(nc(-c4ccccc4)n1-c1ccccc1)B3c1cc(-c3ccccc3)ccc1N2c1ccc(-c2ccccc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccc(-c8ccccc8)cc7c7cc(-c8ccccc8)ccc76)cc3c54)cc2)cc1]

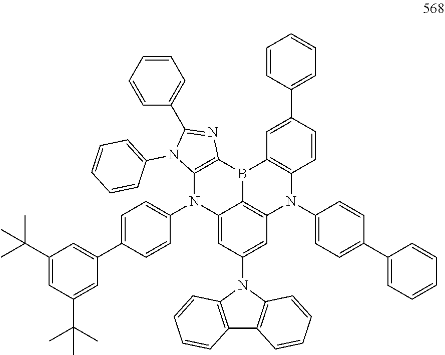 [SMILES: CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4nc(-c5ccccc5)n(-c5ccccc5)c43)cc2)cc(C(C)(C)C)c1]

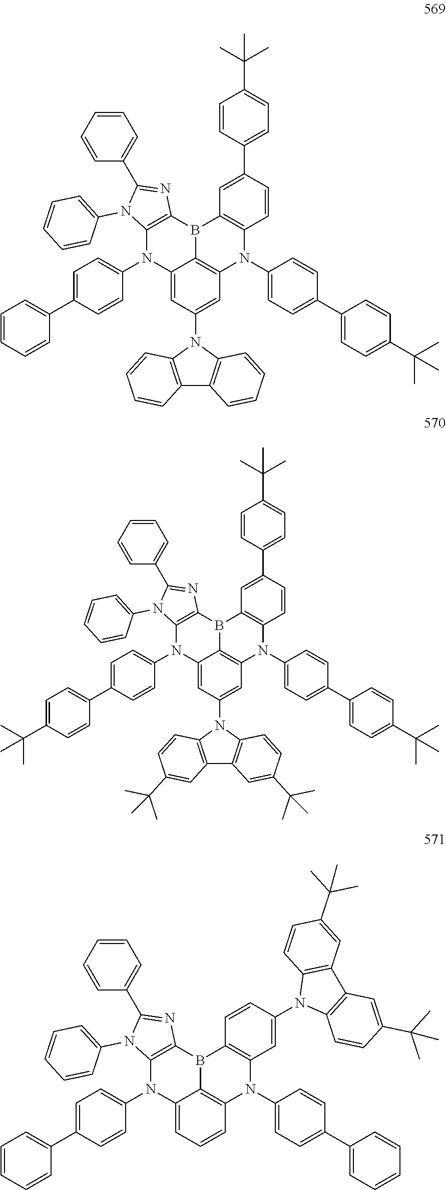

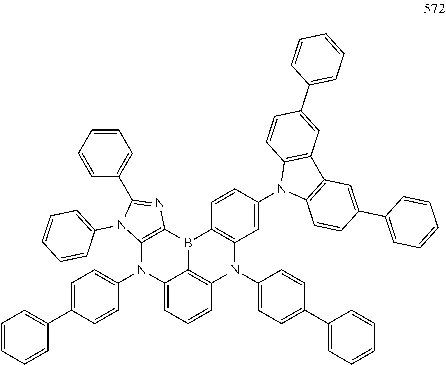 [SMILES: c1ccc(-c2ccc(N3c4cc(-n5c6ccc(-c7ccccc7)cc6c6cc(-c7ccccc7)ccc65)ccc4B4c5nc(-c6ccccc6)n(-c6ccccc6)c5N(c5ccc(-c6ccccc6)cc5)c5cccc3c54)cc2)cc1]

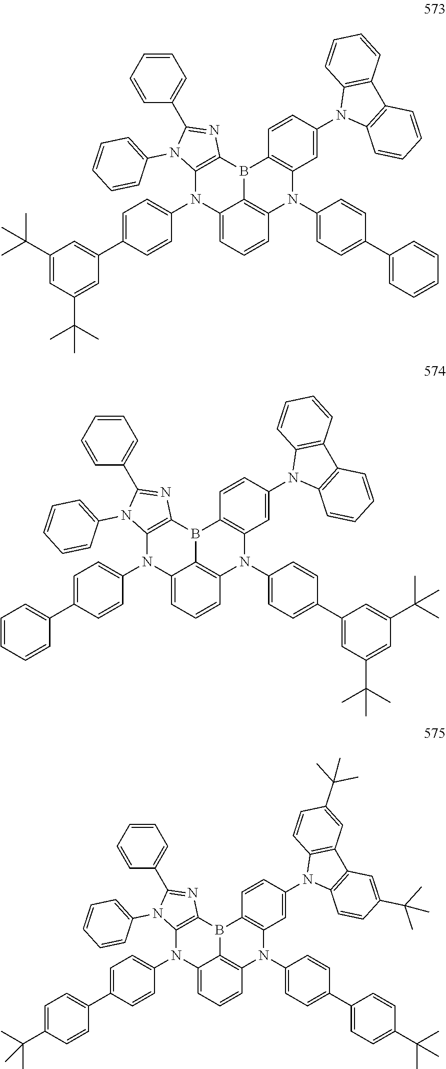

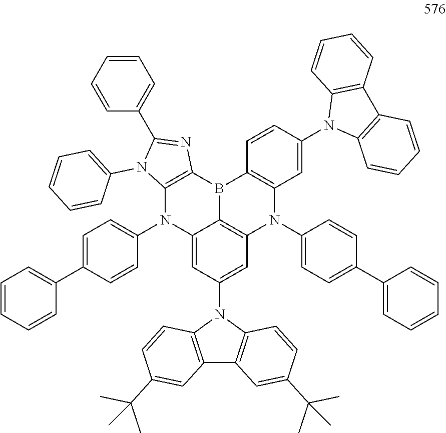 [SMILES: CC(C)(C)c1ccc2c(c1)c1cc(C(C)(C)C)ccc1n2-c1cc2c3c(c1)N(c1ccc(-c4ccccc4)cc1)c1c(nc(-c4ccccc4)n1-c1ccccc1)B3c1ccc(-n3c4ccccc4c4ccccc43)cc1N2c1ccc(-c2ccccc2)cc1]

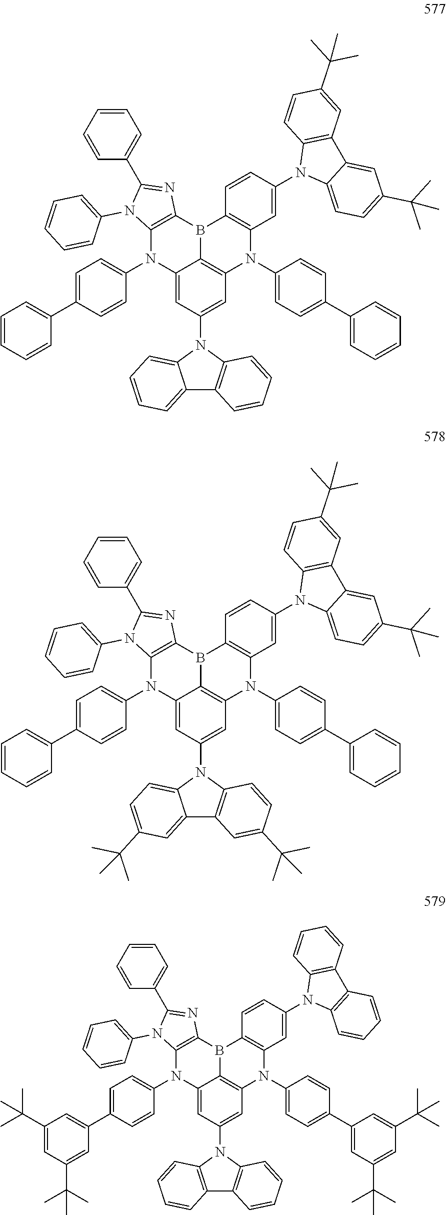

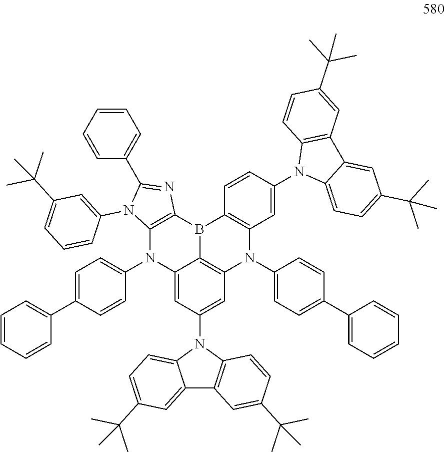 [SMILES: CC(C)(C)c1cccc(-n2c(-c3ccccc3)nc3c2N(c2ccc(-c4ccccc4)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccccc3)cc2)c1]

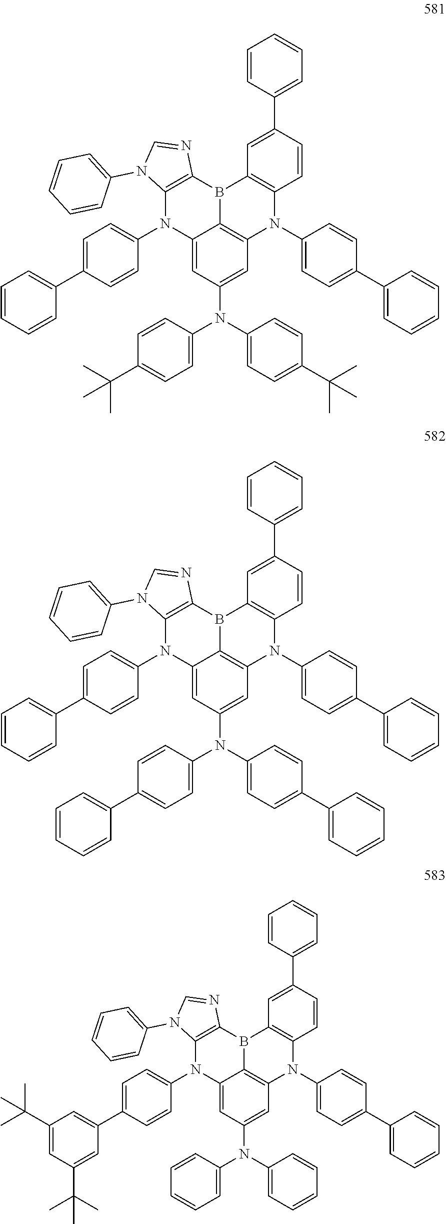 [SMILES: CC(C)(C)c1ccc(N(c2ccc(C(C)(C)C)cc2)c2cc3c4c(c2)N(c2ccc(-c5ccccc5)cc2)c2c(ncn2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(ncn3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4ncn(-c5ccccc5)c43)cc2)cc(C(C)(C)C)c1]

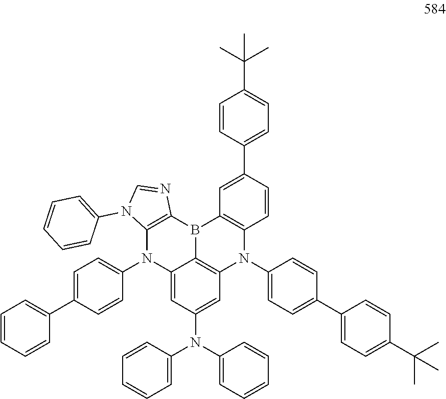 [SMILES: CC(C)(C)c1ccc(-c2ccc(N3c4ccc(-c5ccc(C(C)(C)C)cc5)cc4B4c5ncn(-c6ccccc6)c5N(c5ccc(-c6ccccc6)cc5)c5cc(N(c6ccccc6)c6ccccc6)cc3c54)cc2)cc1]

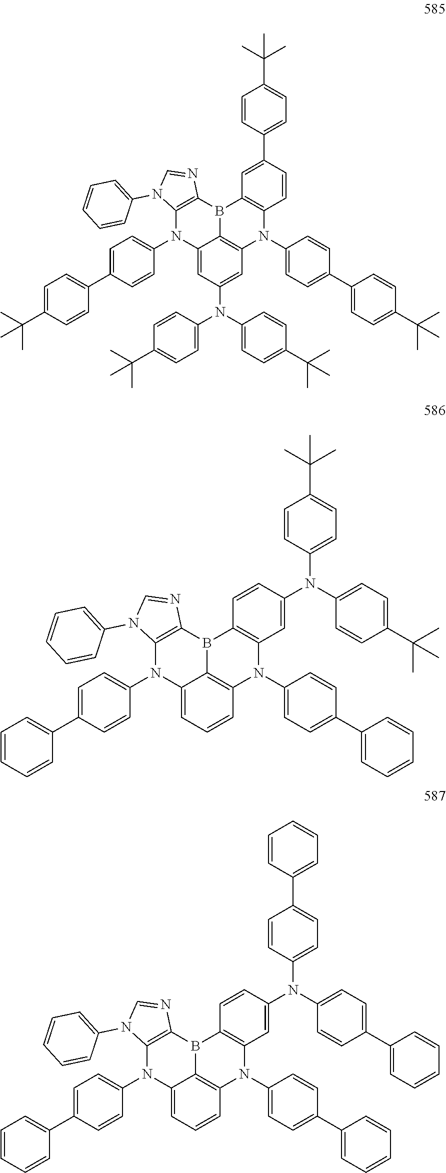 [SMILES: CC(C)(C)c1ccc(-c2ccc(N3c4ccc(-c5ccc(C(C)(C)C)cc5)cc4B4c5ncn(-c6ccccc6)c5N(c5ccc(-c6ccc(C(C)(C)C)cc6)cc5)c5cc(N(c6ccc(C(C)(C)C)cc6)c6ccc(C(C)(C)C)cc6)cc3c54)cc2)cc1 ; CC(C)(C)c1ccc(N(c2ccc(C(C)(C)C)cc2)c2ccc3c(c2)N(c2ccc(-c4ccccc4)cc2)c2cccc4c2B3c2ncn(-c3ccccc3)c2N4c2ccc(-c3ccccc3)cc2)cc1 ; c1ccc(-c2ccc(N(c3ccc(-c4ccccc4)cc3)c3ccc4c(c3)N(c3ccc(-c5ccccc5)cc3)c3cccc5c3B4c3ncn(-c4ccccc4)c3N5c3ccc(-c4ccccc4)cc3)cc2)cc1]

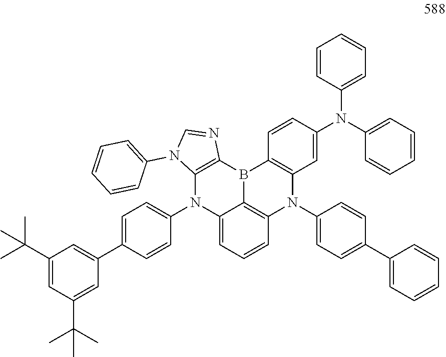 [SMILES: CC(C)(C)c1cc(-c2ccc(N3c4cccc5c4B(c4ccc(N(c6ccccc6)c6ccccc6)cc4N5c4ccc(-c5ccccc5)cc4)c4ncn(-c5ccccc5)c43)cc2)cc(C(C)(C)C)c1]

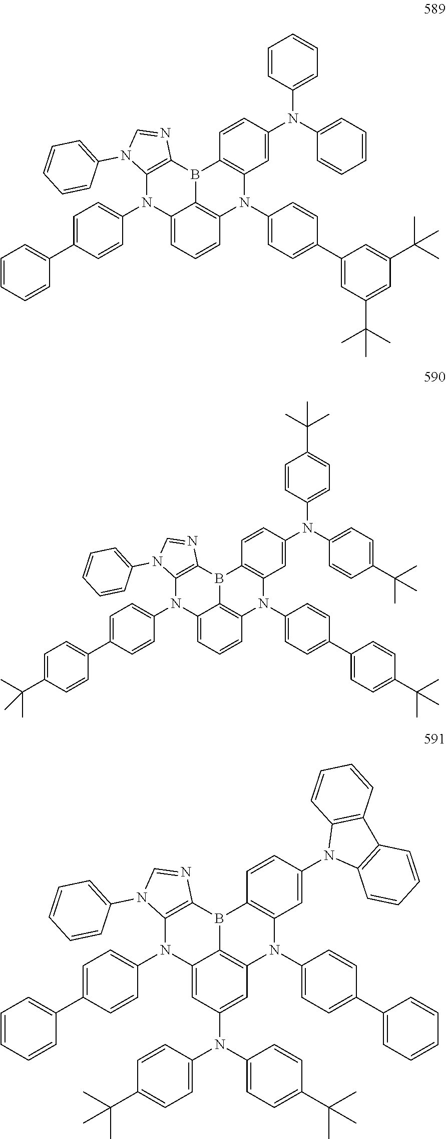

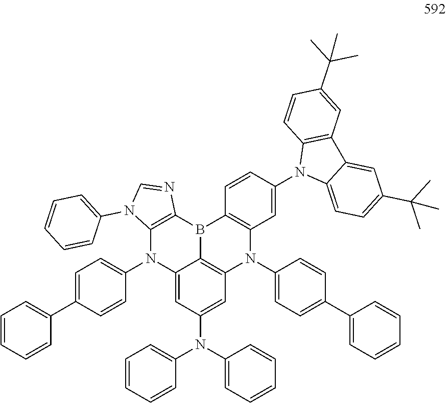 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ncn(-c2ccccc2)c1N3c1ccc(-c2ccccc2)cc1]

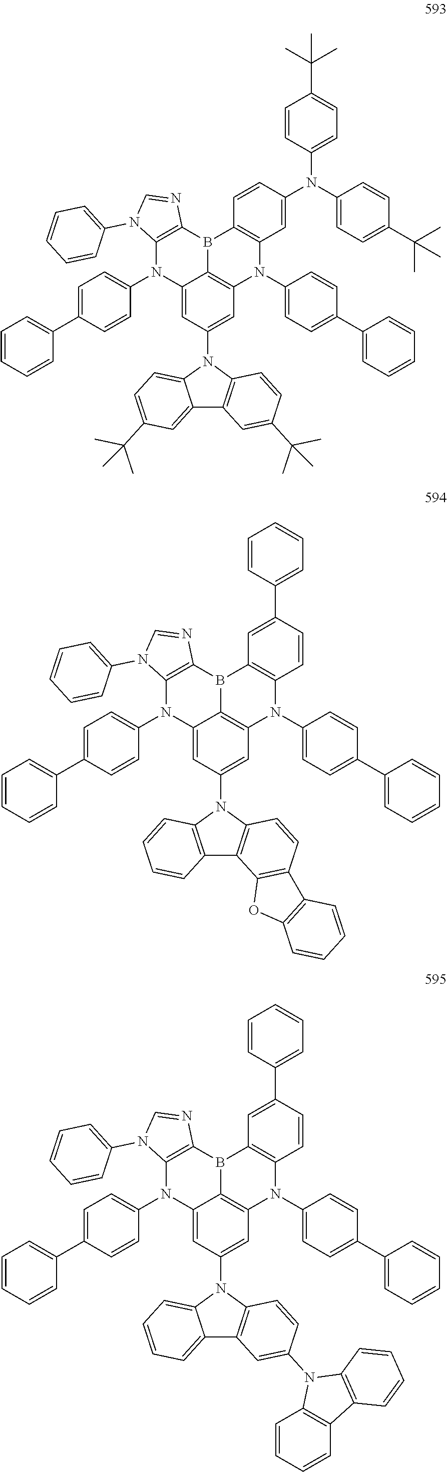 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2ncn(-c3ccccc3)c2N4c2ccc(-c3ccccc3)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-n6c7ccccc7c7c8oc9ccccc9c8ccc76)cc3c54)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-n6c7ccccc7c7cc(-n8c9ccccc9c9ccccc98)ccc76)cc3c54)cc2)cc1]

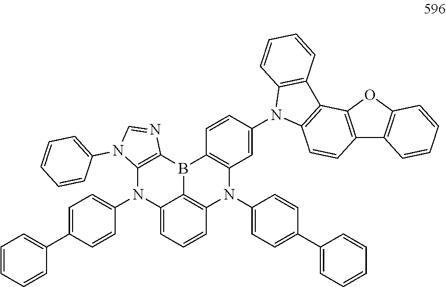 [SMILES: c1ccc(-c2ccc(N3c4cc(-n5c6ccccc6c6c7oc8ccccc8c7ccc65)ccc4B4c5ncn(-c6ccccc6)c5N(c5ccc(-c6ccccc6)cc5)c5cccc3c54)cc2)cc1]

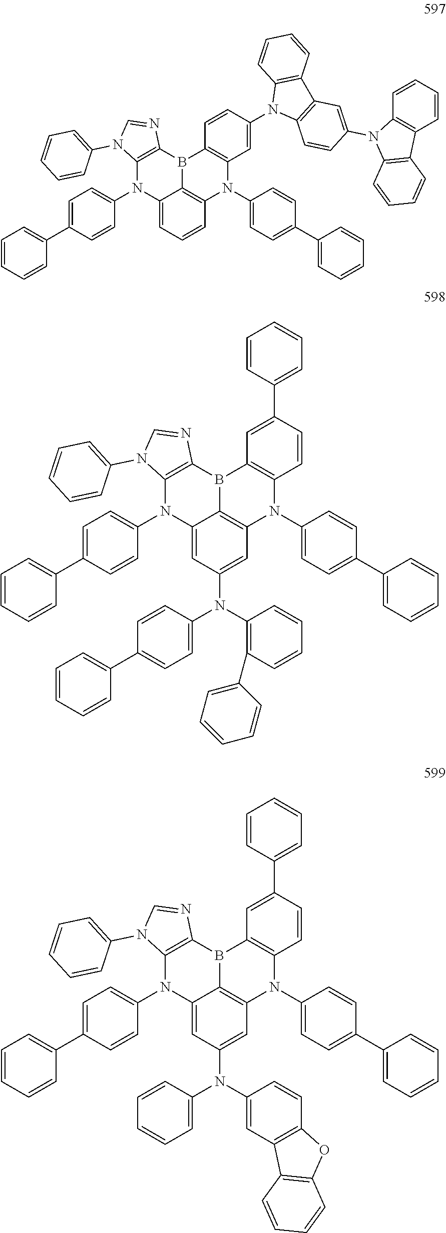 [SMILES: c1ccc(-c2ccc(N3c4cc(-n5c6ccccc6c6cc(-n7c8ccccc8c8ccccc87)ccc65)ccc4B4c5ncn(-c6ccccc6)c5N(c5ccc(-c6ccccc6)cc5)c5cccc3c54)cc2)cc1 ; c1ccc(-c2ccc(N(c3cc4c5c(c3)N(c3ccc(-c6ccccc6)cc3)c3c(ncn3-c3ccccc3)B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5ncn(-c6ccccc6)c5N(c5ccc(-c6ccccc6)cc5)c5cc(N(c6ccccc6)c6ccc7oc8ccccc8c7c6)cc3c54)cc2)cc1]

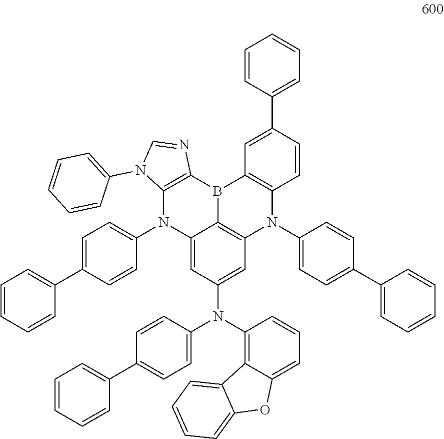 [SMILES: c1ccc(-c2ccc(N3c4ccc(-c5ccccc5)cc4B4c5ncn(-c6ccccc6)c5N(c5ccc(-c6ccccc6)cc5)c5cc(N(c6ccc(-c7ccccc7)cc6)c6cccc7oc8ccccc8c67)cc3c54)cc2)cc1]

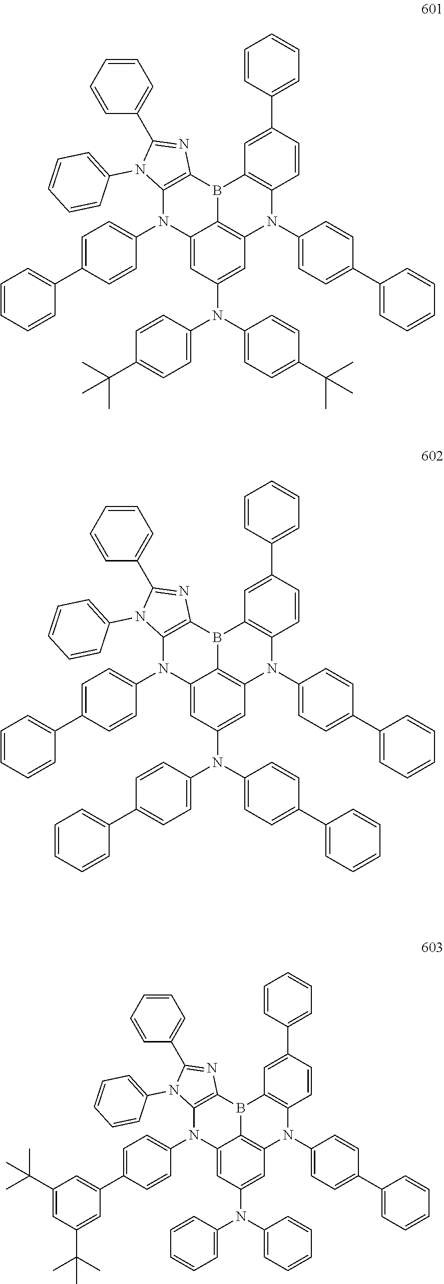 [SMILES: CC(C)(C)c1ccc(N(c2ccc(C(C)(C)C)cc2)c2cc3c4c(c2)N(c2ccc(-c5ccccc5)cc2)c2c(nc(-c5ccccc5)n2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(nc(-c6ccccc6)n3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4nc(-c5ccccc5)n(-c5ccccc5)c43)cc2)cc(C(C)(C)C)c1]

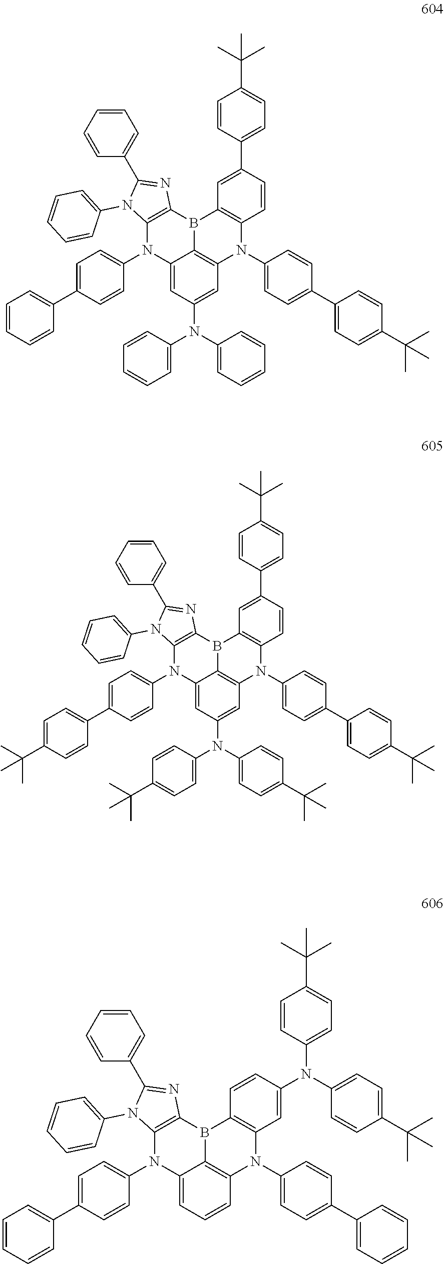

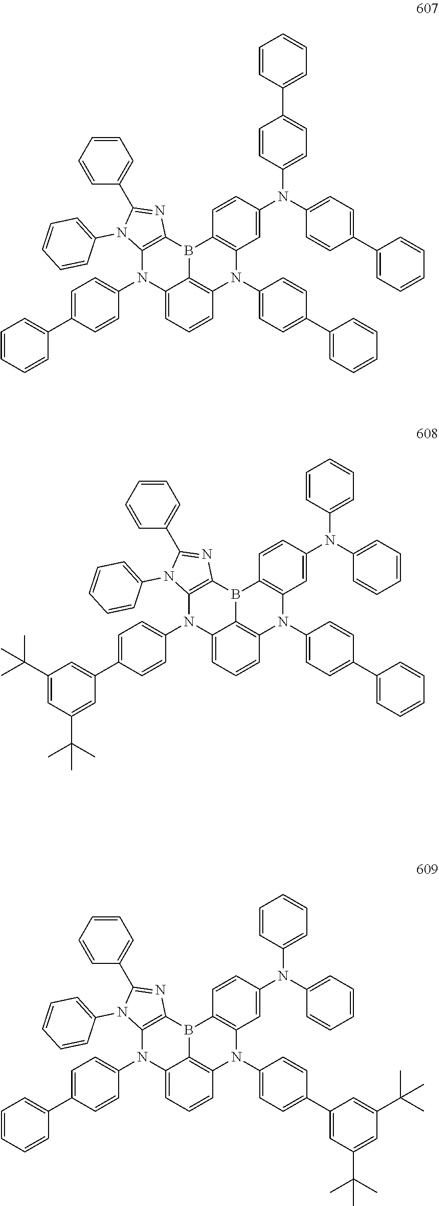 [SMILES: c1ccc(-c2ccc(N(c3ccc(-c4ccccc4)cc3)c3ccc4c(c3)N(c3ccc(-c5ccccc5)cc3)c3cccc5c3B4c3nc(-c4ccccc4)n(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4nc(-c5ccccc5)n(-c5ccccc5)c43)cc2)cc(C(C)(C)C)c1 ; CC(C)(C)c1cc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cccc3c54)cc2)cc(C(C)(C)C)c1]

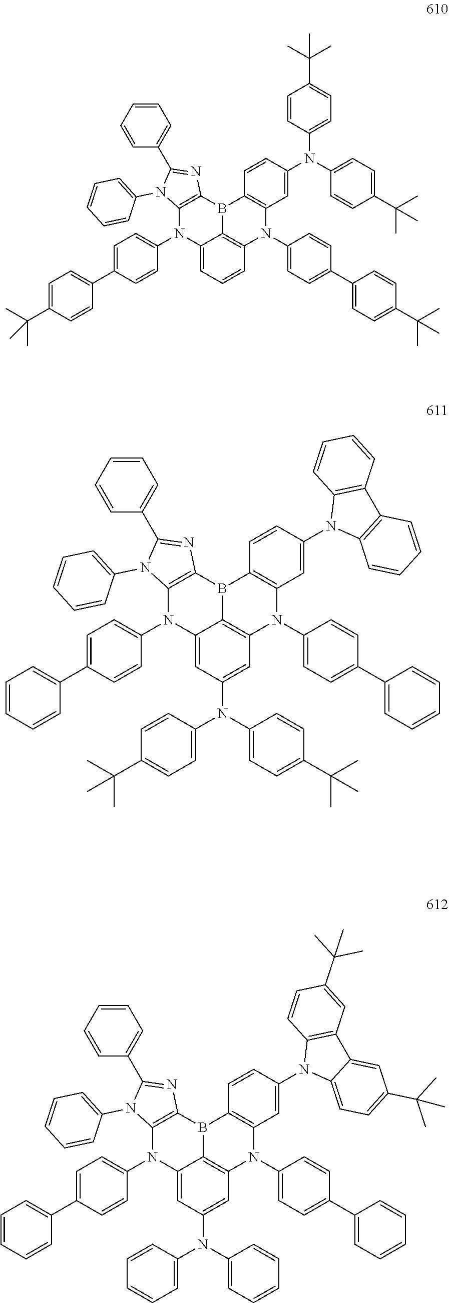 [SMILES: CC(C)(C)c1ccc(-c2ccc(N3c4cc(N(c5ccc(C(C)(C)C)cc5)c5ccc(C(C)(C)C)cc5)ccc4B4c5nc(-c6ccccc6)n(-c6ccccc6)c5N(c5ccc(-c6ccc(C(C)(C)C)cc6)cc5)c5cccc3c54)cc2)cc1 ; CC(C)(C)c1ccc(N(c2ccc(C(C)(C)C)cc2)c2cc3c4c(c2)N(c2ccc(-c5ccccc5)cc2)c2c(nc(-c5ccccc5)n2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1nc(-c2ccccc2)n(-c2ccccc2)c1N3c1ccc(-c2ccccc2)cc1]

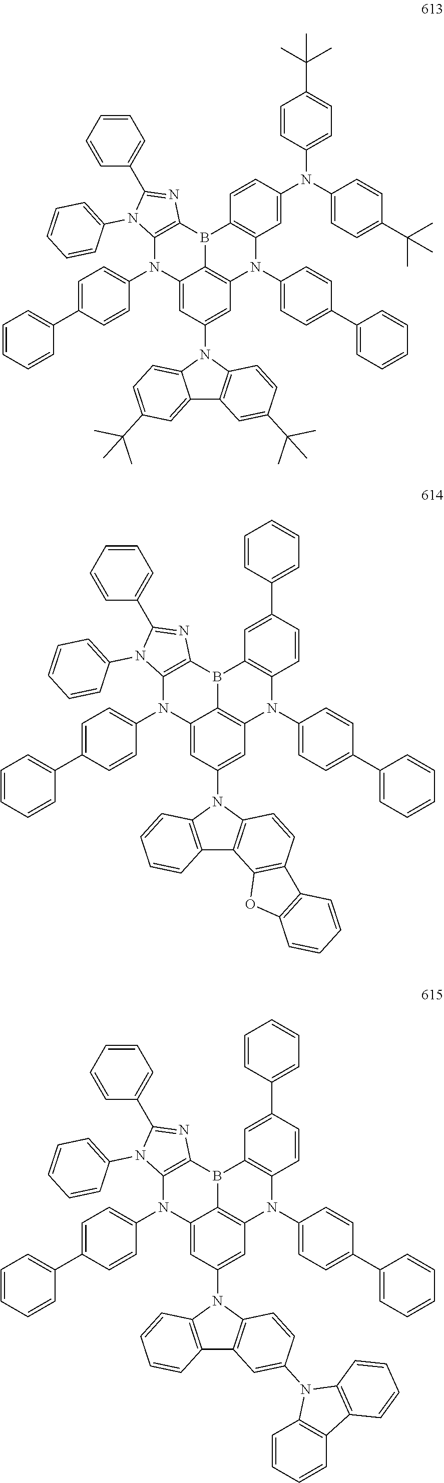 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2nc(-c3ccccc3)n(-c3ccccc3)c2N4c2ccc(-c3ccccc3)cc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7c8oc9ccccc9c8ccc76)cc3c54)cc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7cc(-n8c9ccccc9c9ccccc98)ccc76)cc3c54)cc2)cc1]

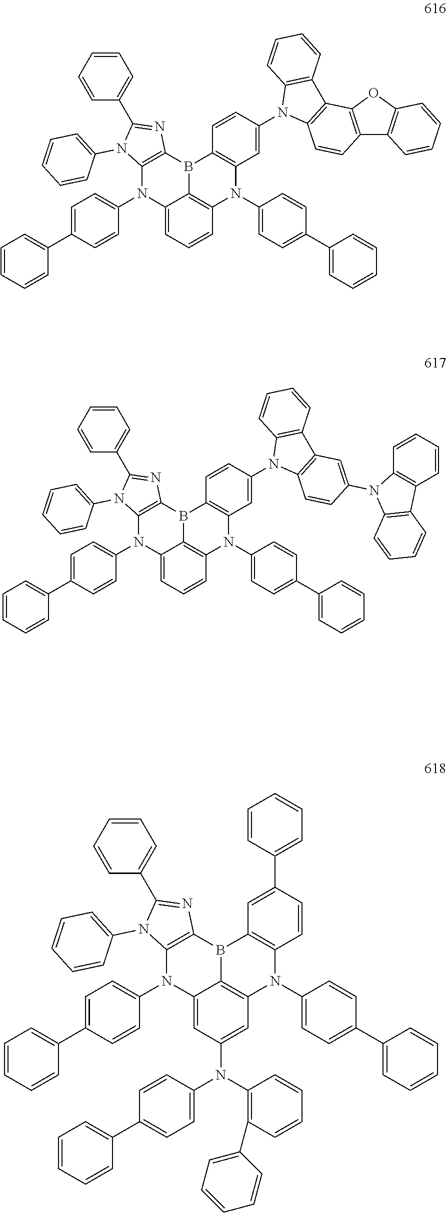 [SMILES: c1ccc(-c2ccc(N3c4cc(-n5c6ccccc6c6c7oc8ccccc8c7ccc65)ccc4B4c5nc(-c6ccccc6)n(-c6ccccc6)c5N(c5ccc(-c6ccccc6)cc5)c5cccc3c54)cc2)cc1 ; c1ccc(-c2ccc(N3c4cc(-n5c6ccccc6c6cc(-n7c8ccccc8c8ccccc87)ccc65)ccc4B4c5nc(-c6ccccc6)n(-c6ccccc6)c5N(c5ccc(-c6ccccc6)cc5)c5cccc3c54)cc2)cc1 ; c1ccc(-c2ccc(N(c3cc4c5c(c3)N(c3ccc(-c6ccccc6)cc3)c3c(nc(-c6ccccc6)n3-c3ccccc3)B5c3cc(-c5ccccc5)ccc3N4c3ccc(-c4ccccc4)cc3)c3ccccc3-c3ccccc3)cc2)cc1]

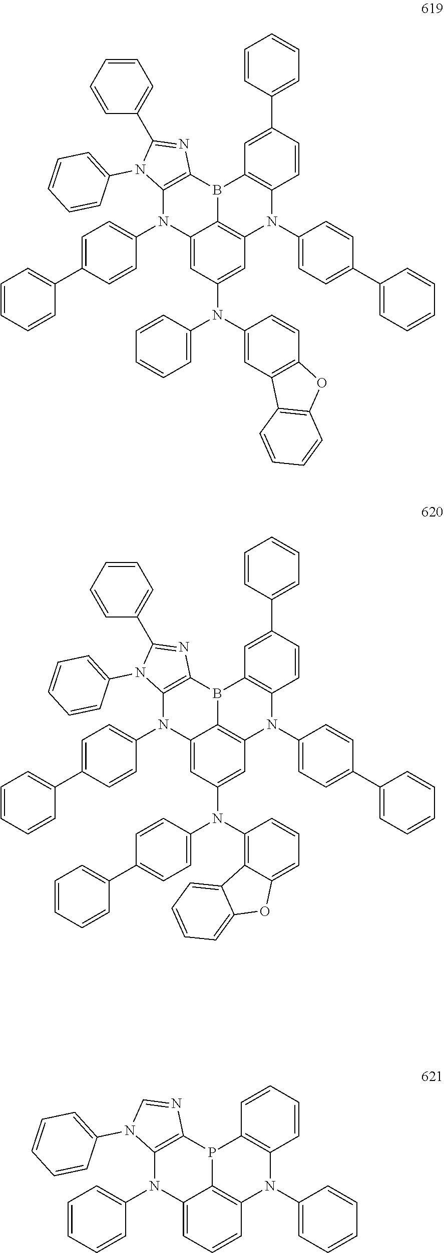 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(N(c6ccccc6)c6ccc7oc8ccccc8c7c6)cc3c54)cc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(N(c6ccc(-c7ccccc7)cc6)c6cccc7oc8ccccc8c67)cc3c54)cc2)cc1 ; c1ccc(N2c3ccccc3P3c4ncn(-c5ccccc5)c4N(c4ccccc4)c4cccc2c43)cc1]

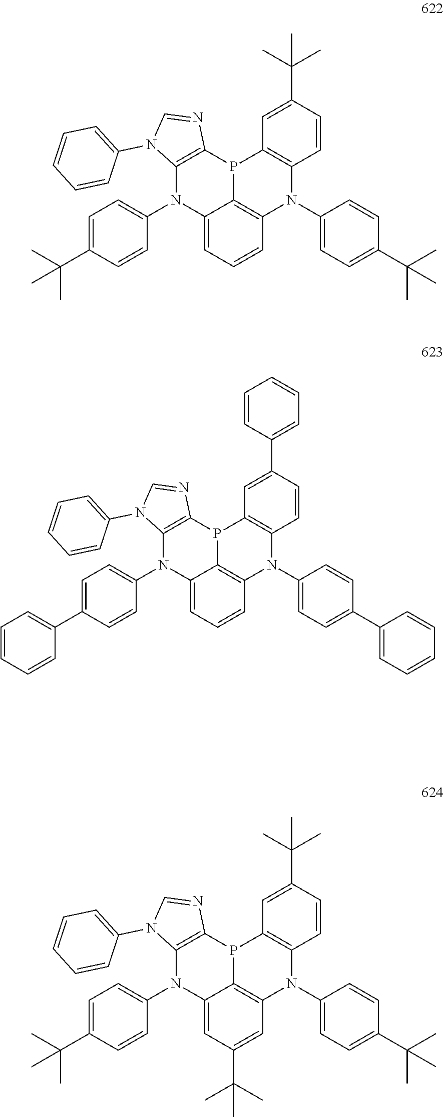

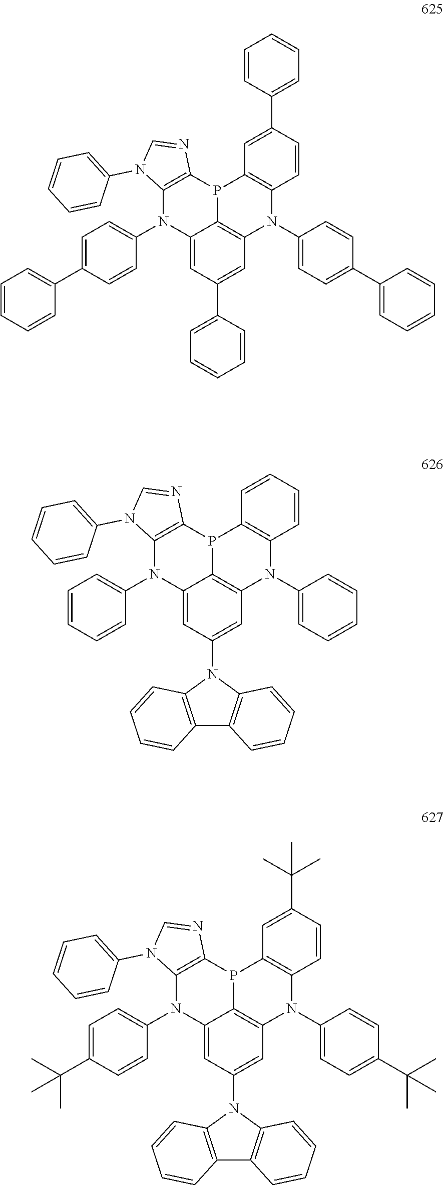 [SMILES: c1ccc(-c2ccc(N3c4ccc(-c5ccccc5)cc4P4c5ncn(-c6ccccc6)c5N(c5ccc(-c6ccccc6)cc5)c5cc(-c6ccccc6)cc3c54)cc2)cc1 ; c1ccc(N2c3ccccc3P3c4ncn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3P3c4ncn(-c5ccccc5)c4N(c4ccc(C(C)(C)C)cc4)c4cc(-n5c6ccccc6c6ccccc65)cc2c43)cc1]

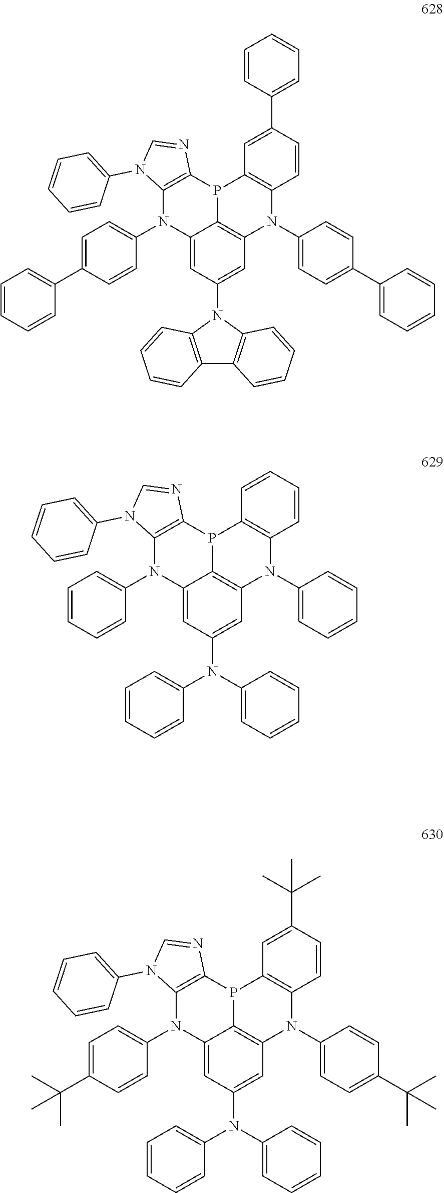 [SMILES: c1ccc(-c2ccc(N3c4ccc(-c5ccccc5)cc4P4c5ncn(-c6ccccc6)c5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2c(ncn2-c2ccccc2)P4c2ccccc2N3c2ccccc2)cc1 ; CC(C)(C)c1ccc(N2c3ccc(C(C)(C)C)cc3P3c4ncn(-c5ccccc5)c4N(c4ccc(C(C)(C)C)cc4)c4cc(N(c5ccccc5)c5ccccc5)cc2c43)cc1]

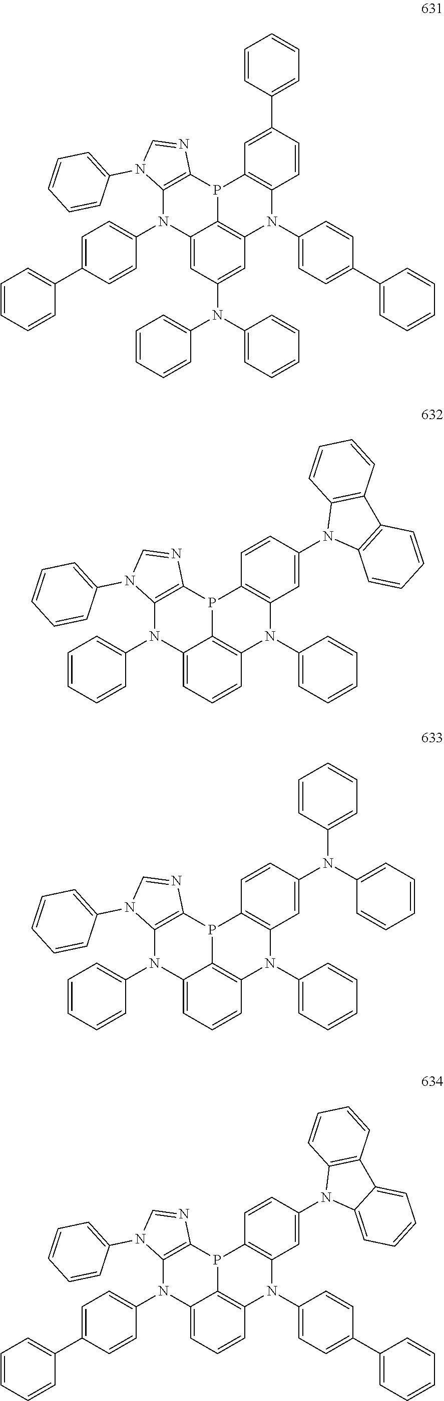 [SMILES: c1ccc(-c2ccc(N3c4ccc(-c5ccccc5)cc4P4c5ncn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3P3c4ncn(-c5ccccc5)c4N(c4ccccc4)c4cccc2c43)cc1 ; c1ccc(N(c2ccccc2)c2ccc3c(c2)N(c2ccccc2)c2cccc4c2P3c2ncn(-c3ccccc3)c2N4c2ccccc2)cc1 ; c1ccc(-c2ccc(N3c4cc(-n5c6ccccc6c6ccccc65)ccc4P4c5ncn(-c6ccccc6)c5N(c5ccc(-c6ccccc6)cc5)c5cccc3c54)cc2)cc1]

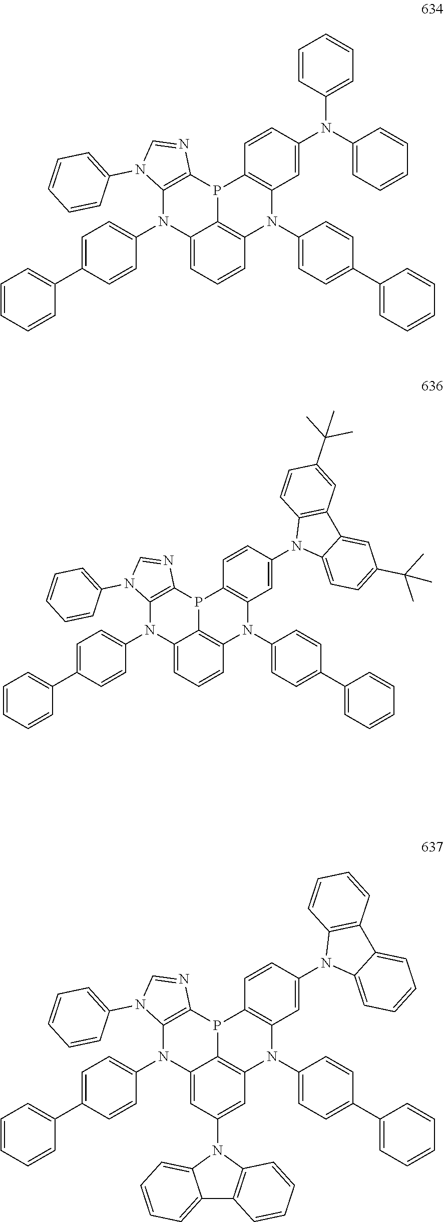 [SMILES: c1ccc(-c2ccc(N3c4cc(N(c5ccccc5)c5ccccc5)ccc4P4c5ncn(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1ncn(-c2ccccc2)c1N3c1ccc(-c2ccccc2)cc1 ; c1ccc(-c2ccc(N3c4cc(-n5c6ccccc6c6ccccc65)ccc4P4c5ncn(-c6ccccc6)c5N(c5ccc(-c6ccccc6)cc5)c5cc(-n6c7ccccc7c7ccccc76)cc3c54)cc2)cc1]

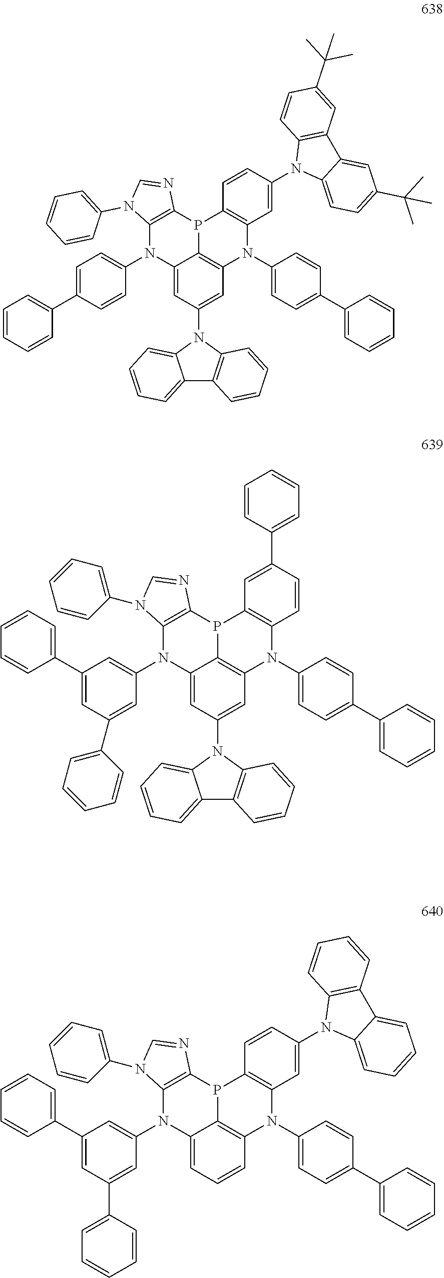 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1ncn(-c2ccccc2)c1N3c1ccc(-c2ccccc2)cc1 ; c1ccc(-c2ccc(N3c4ccc(-c5ccccc5)cc4P4c5ncn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4P4c5ncn(-c6ccccc6)c5N(c5cc(-c6ccccc6)cc(-c6ccccc6)c5)c5cccc3c54)cc2)cc1]

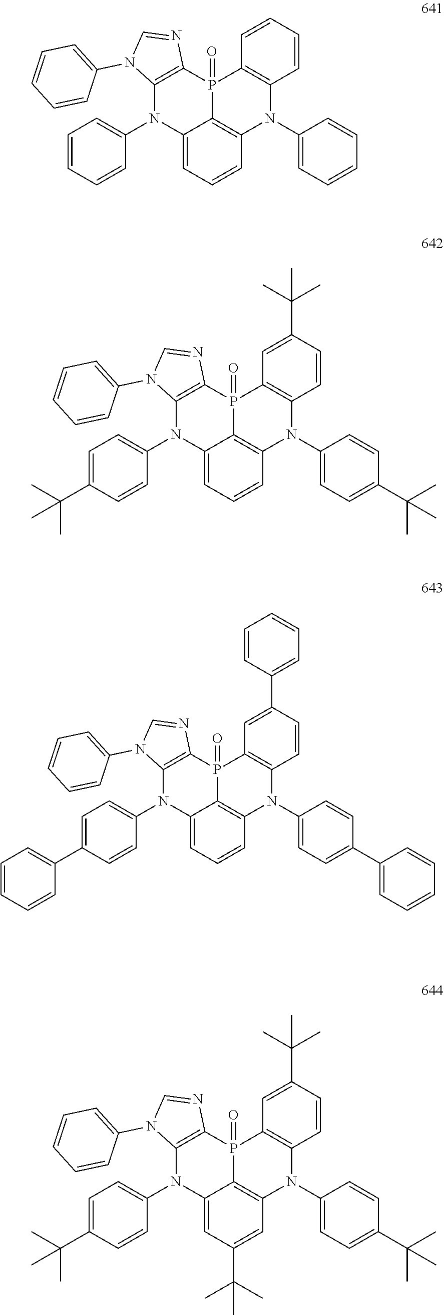

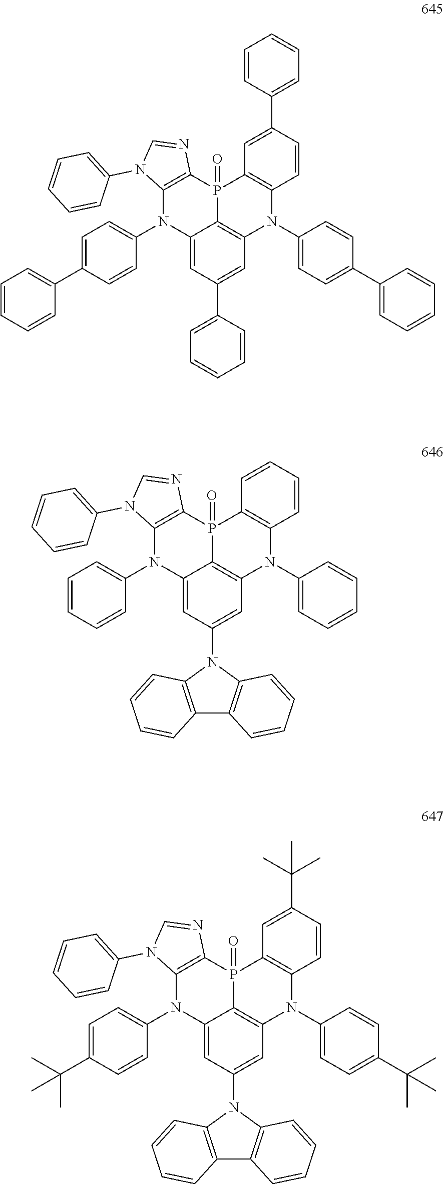 [SMILES: O=P12c3cc(-c4ccccc4)ccc3N(c3ccc(-c4ccccc4)cc3)c3cc(-c4ccccc4)cc(c31)N(c1ccc(-c3ccccc3)cc1)c1c2ncn1-c1ccccc1 ; O=P12c3ccccc3N(c3ccccc3)c3cc(-n4c5ccccc5c5ccccc54)cc(c31)N(c1ccccc1)c1c2ncn1-c1ccccc1 ; CC(C)(C)c1ccc(N2c3ccc(C(C)(C)C)cc3P3(=O)c4ncn(-c5ccccc5)c4N(c4ccc(C(C)(C)C)cc4)c4cc(-n5c6ccccc6c6ccccc65)cc2c43)cc1]

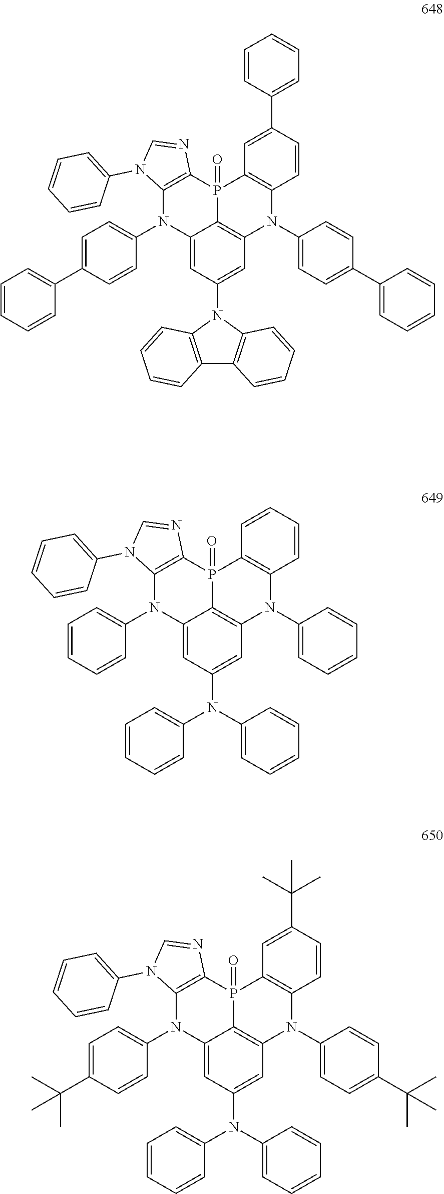 [SMILES: O=P12c3cc(-c4ccccc4)ccc3N(c3ccc(-c4ccccc4)cc3)c3cc(-n4c5ccccc5c5ccccc54)cc(c31)N(c1ccc(-c3ccccc3)cc1)c1c2ncn1-c1ccccc1 ; O=P12c3ccccc3N(c3ccccc3)c3cc(N(c4ccccc4)c4ccccc4)cc(c31)N(c1ccccc1)c1c2ncn1-c1ccccc1 ; CC(C)(C)c1ccc(N2c3ccc(C(C)(C)C)cc3P3(=O)c4ncn(-c5ccccc5)c4N(c4ccc(C(C)(C)C)cc4)c4cc(N(c5ccccc5)c5ccccc5)cc2c43)cc1]

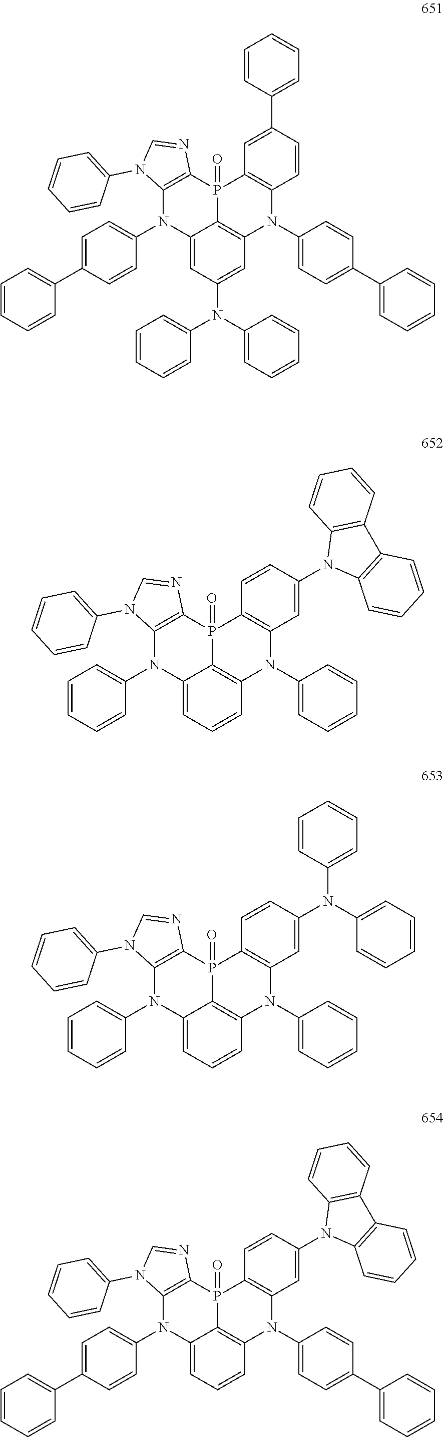 [SMILES: O=P12c3cc(-c4ccccc4)ccc3N(c3ccc(-c4ccccc4)cc3)c3cc(N(c4ccccc4)c4ccccc4)cc(c31)N(c1ccc(-c3ccccc3)cc1)c1c2ncn1-c1ccccc1 ; O=P12c3ccc(-n4c5ccccc5c5ccccc54)cc3N(c3ccccc3)c3cccc(c31)N(c1ccccc1)c1c2ncn1-c1ccccc1 ; O=P12c3ccc(N(c4ccccc4)c4ccccc4)cc3N(c3ccccc3)c3cccc(c31)N(c1ccccc1)c1c2ncn1-c1ccccc1 ; O=P12c3ccc(-n4c5ccccc5c5ccccc54)cc3N(c3ccc(-c4ccccc4)cc3)c3cccc(c31)N(c1ccc(-c3ccccc3)cc1)c1c2ncn1-c1ccccc1]

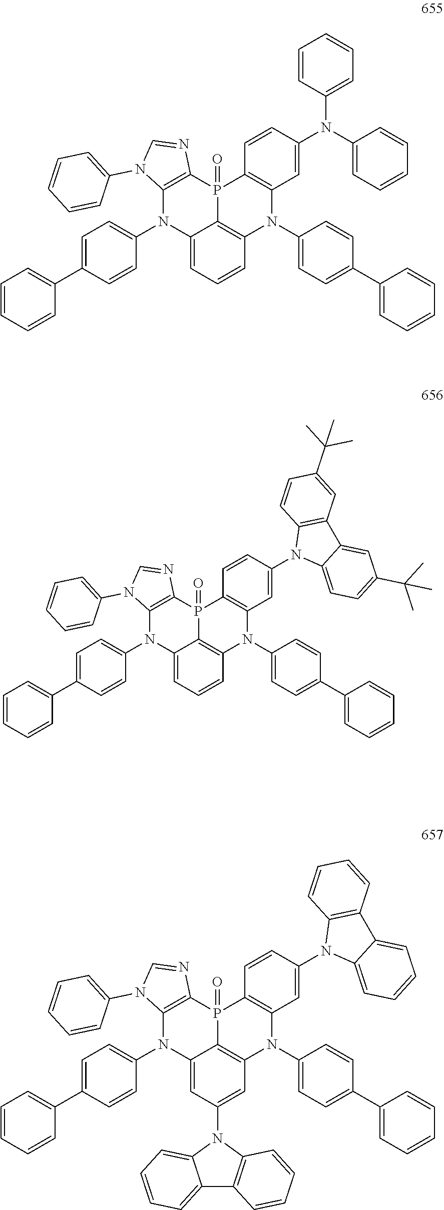 [SMILES: O=P12c3ccc(N(c4ccccc4)c4ccccc4)cc3N(c3ccc(-c4ccccc4)cc3)c3cccc(c31)N(c1ccc(-c3ccccc3)cc1)c1c2ncn1-c1ccccc1 ; CC(C)(C)c1ccc2c(c1)c1cc(C(C)(C)C)ccc1n2-c1ccc2c(c1)N(c1ccc(-c3ccccc3)cc1)c1cccc3c1P2(=O)c1ncn(-c2ccccc2)c1N3c1ccc(-c2ccccc2)cc1 ; O=P12c3ccc(-n4c5ccccc5c5ccccc54)cc3N(c3ccc(-c4ccccc4)cc3)c3cc(-n4c5ccccc5c5ccccc54)cc(c31)N(c1ccc(-c3ccccc3)cc1)c1c2ncn1-c1ccccc1]

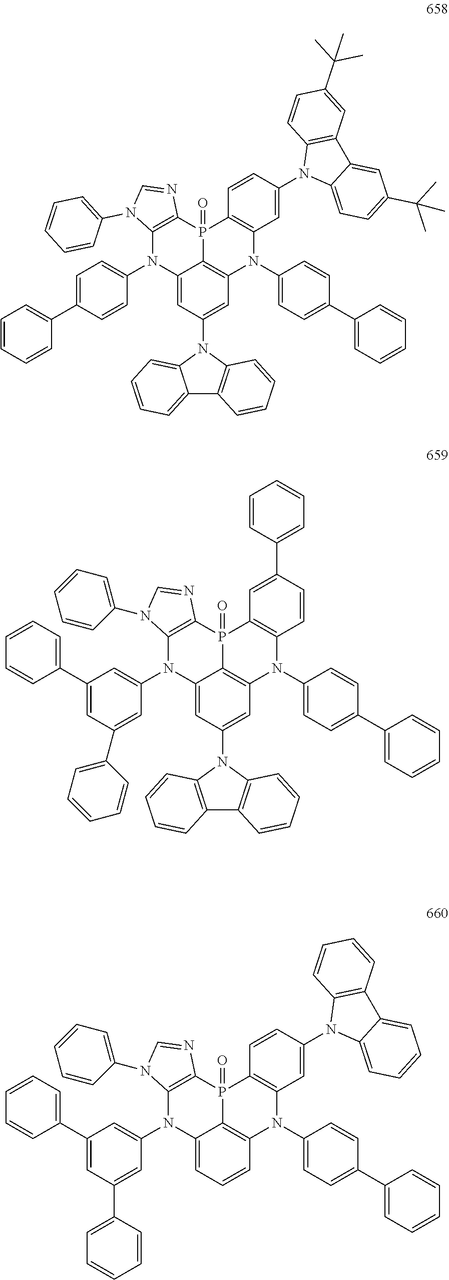 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2(=O)c1ncn(-c2ccccc2)c1N3c1ccc(-c2ccccc2)cc1 ; O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1c2ncn1-c1ccccc1 ; O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1c2ncn1-c1ccccc1]

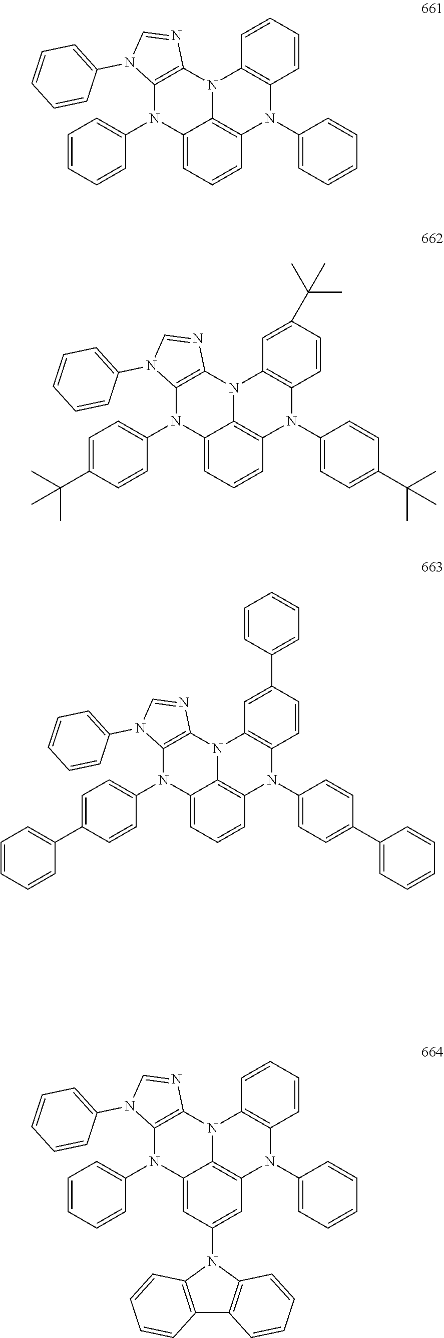 [SMILES: c1ccc(N2c3ccccc3N3c4ncn(-c5ccccc5)c4N(c4ccccc4)c4cccc2c43)cc1 ; CC(C)(C)c1ccc(N2c3ccc(C(C)(C)C)cc3N3c4ncn(-c5ccccc5)c4N(c4ccc(C(C)(C)C)cc4)c4cccc2c43)cc1 ; c1ccc(-c2ccc(N3c4ccc(-c5ccccc5)cc4N4c5ncn(-c6ccccc6)c5N(c5ccc(-c6ccccc6)cc5)c5cccc3c54)cc2)cc1 ; c1ccc(N2c3ccccc3N3c4ncn(-c5ccccc5)c4N(c4ccccc4)c4cc(-n5c6ccccc6c6ccccc65)cc2c43)cc1]

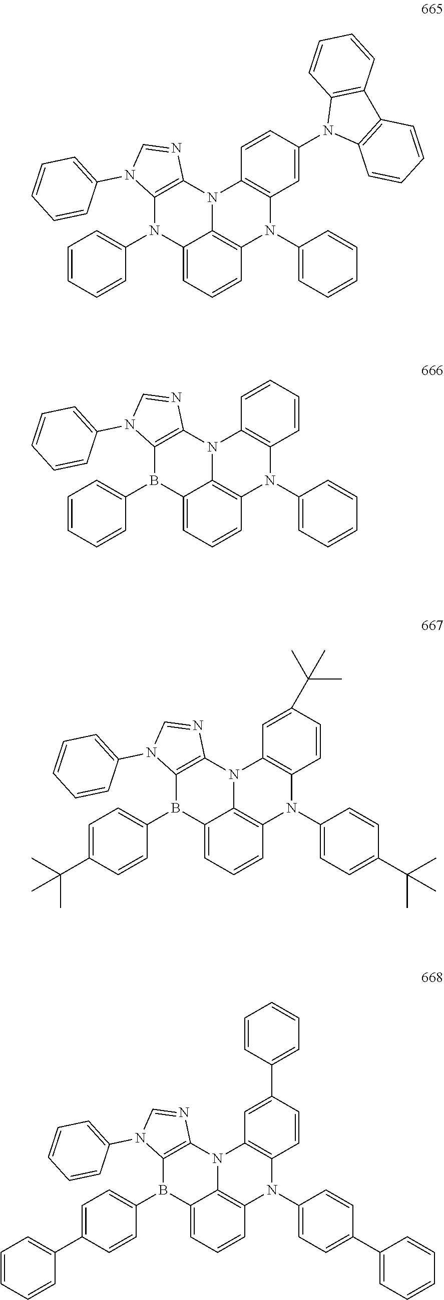 [SMILES: c1ccc(N2c3cc(-n4c5ccccc5c5ccccc54)ccc3N3c4ncn(-c5ccccc5)c4N(c4ccccc4)c4cccc2c43)cc1 ; c1ccc(B2c3cccc4c3N(c3ccccc3N4c3ccccc3)c3ncn(-c4ccccc4)c32)cc1 ; CC(C)(C)c1ccc(B2c3cccc4c3N(c3cc(C(C)(C)C)ccc3N4c3ccc(C(C)(C)C)cc3)c3ncn(-c4ccccc4)c32)cc1 ; c1ccc(-c2ccc(B3c4cccc5c4N(c4cc(-c6ccccc6)ccc4N5c4ccc(-c5ccccc5)cc4)c4ncn(-c5ccccc5)c43)cc2)cc1]

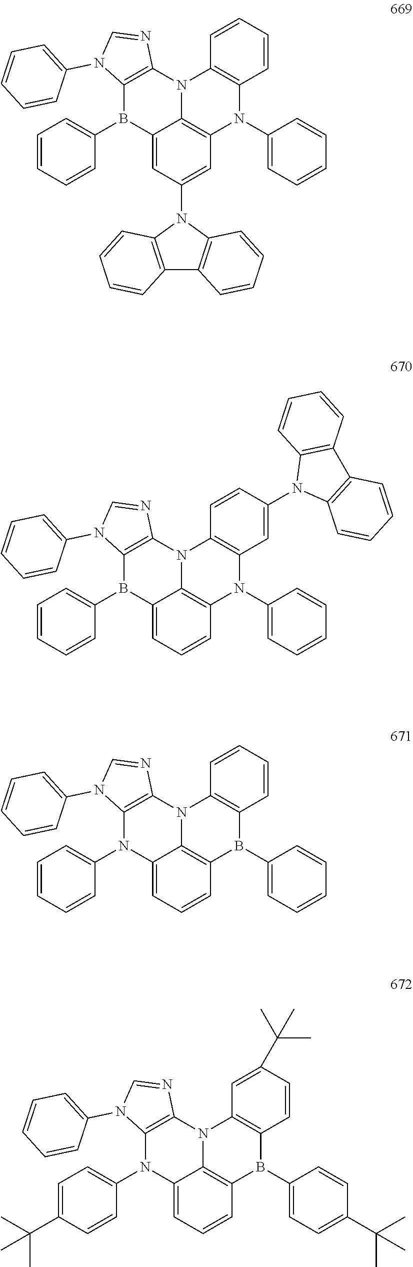 [SMILES: c1ccc(B2c3cc(-n4c5ccccc5c5ccccc54)cc4c3N(c3ccccc3N4c3ccccc3)c3ncn(-c4ccccc4)c32)cc1 ; c1ccc(B2c3cccc4c3N(c3ccc(-n5c6ccccc6c6ccccc65)cc3N4c3ccccc3)c3ncn(-c4ccccc4)c32)cc1 ; c1ccc(B2c3ccccc3N3c4ncn(-c5ccccc5)c4N(c4ccccc4)c4cccc2c43)cc1 ; CC(C)(C)c1ccc(B2c3ccc(C(C)(C)C)cc3N3c4ncn(-c5ccccc5)c4N(c4ccc(C(C)(C)C)cc4)c4cccc2c43)cc1]

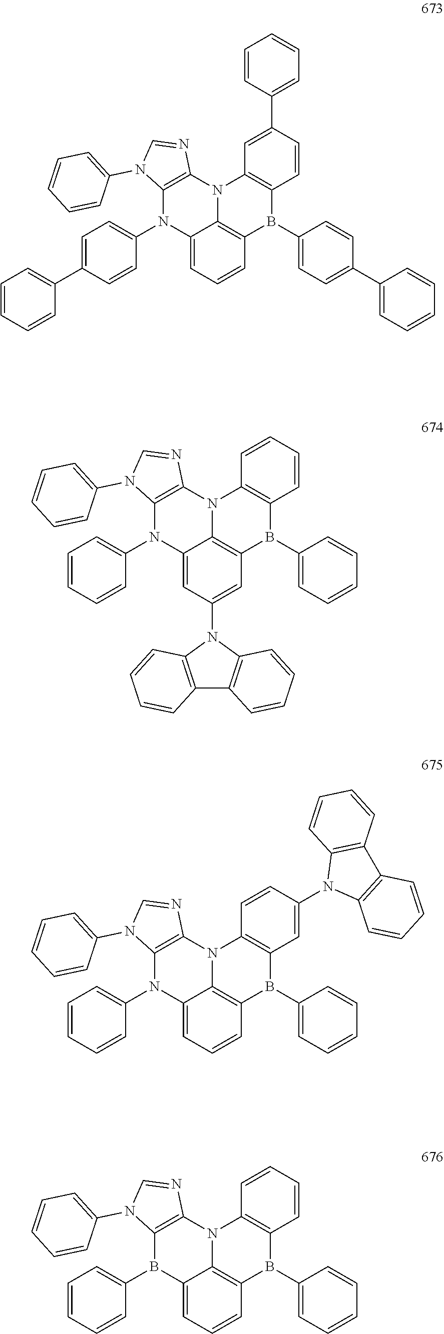 [SMILES: c1ccc(-c2ccc(B3c4ccc(-c5ccccc5)cc4N4c5ncn(-c6ccccc6)c5N(c5ccc(-c6ccccc6)cc5)c5cccc3c54)cc2)cc1 ; c1ccc(B2c3ccccc3N3c4ncn(-c5ccccc5)c4N(c4ccccc4)c4cc(-n5c6ccccc6c6ccccc65)cc2c43)cc1 ; c1ccc(B2c3cc(-n4c5ccccc5c5ccccc54)ccc3N3c4ncn(-c5ccccc5)c4N(c4ccccc4)c4cccc2c43)cc1 ; c1ccc(B2c3ccccc3N3c4ncn(-c5ccccc5)c4B(c4ccccc4)c4cccc2c43)cc1]

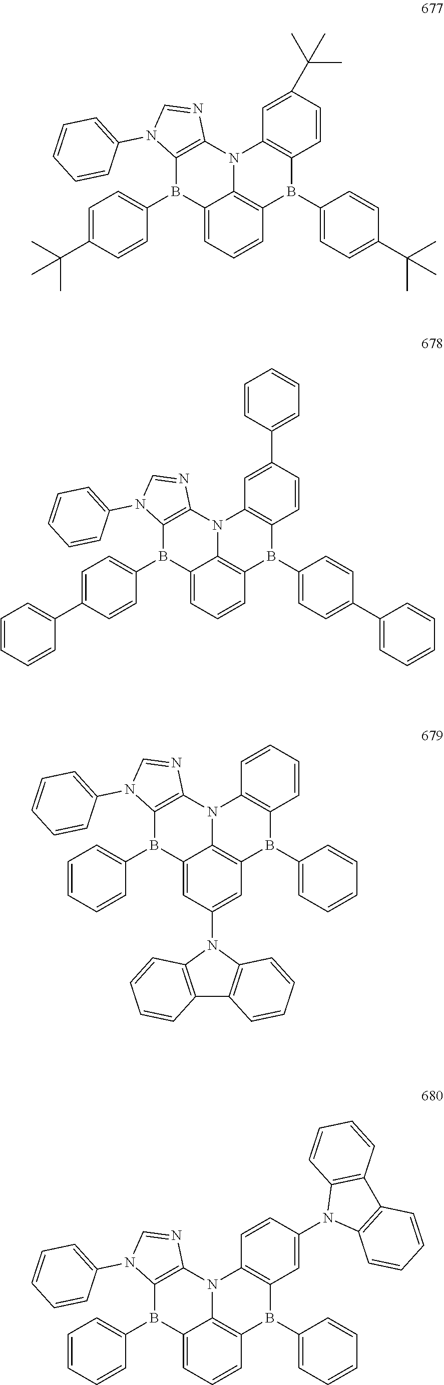 [SMILES: CC(C)(C)c1ccc(B2c3ccc(C(C)(C)C)cc3N3c4ncn(-c5ccccc5)c4B(c4ccc(C(C)(C)C)cc4)c4cccc2c43)cc1 ; c1ccc(-c2ccc(B3c4ccc(-c5ccccc5)cc4N4c5ncn(-c6ccccc6)c5B(c5ccc(-c6ccccc6)cc5)c5cccc3c54)cc2)cc1 ; c1ccc(B2c3ccccc3N3c4ncn(-c5ccccc5)c4B(c4ccccc4)c4cc(-n5c6ccccc6c6ccccc65)cc2c43)cc1 ; c1ccc(B2c3cc(-n4c5ccccc5c5ccccc54)ccc3N3c4ncn(-c5ccccc5)c4B(c4ccccc4)c4cccc2c43)cc1]

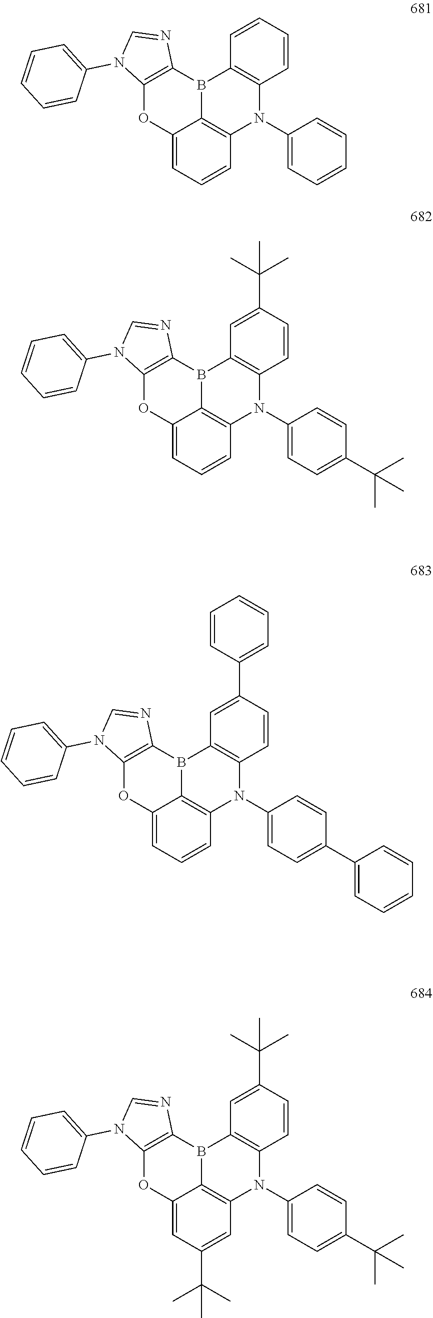 [SMILES: c1ccc(N2c3ccccc3B3c4ncn(-c5ccccc5)c4Oc4cccc2c43)cc1 ; CC(C)(C)c1ccc(N2c3ccc(C(C)(C)C)cc3B3c4ncn(-c5ccccc5)c4Oc4cccc2c43)cc1 ; c1ccc(-c2ccc(N3c4ccc(-c5ccccc5)cc4B4c5ncn(-c6ccccc6)c5Oc5cccc3c54)cc2)cc1 ; CC(C)(C)c1ccc(N2c3ccc(C(C)(C)C)cc3B3c4ncn(-c5ccccc5)c4Oc4cc(C(C)(C)C)cc2c43)cc1]

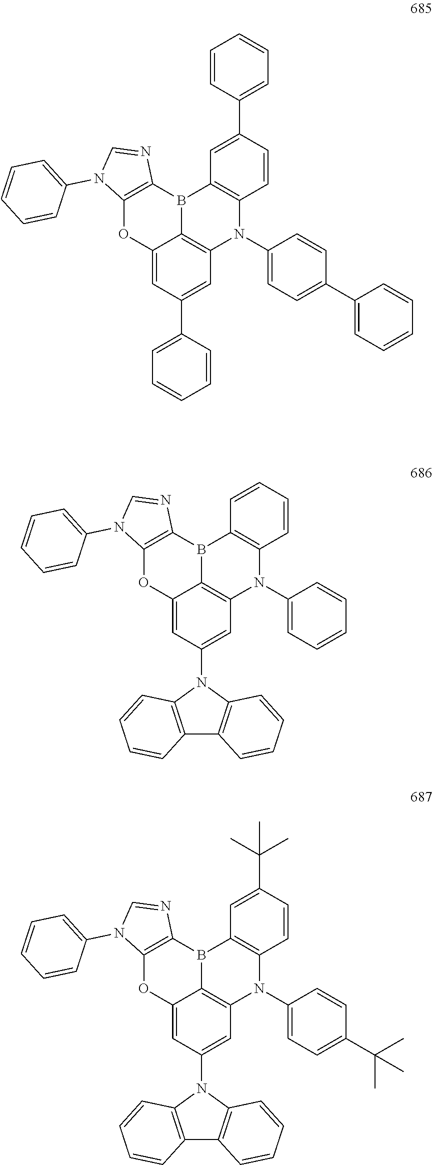 [SMILES: c1ccc(-c2ccc(N3c4ccc(-c5ccccc5)cc4B4c5ncn(-c6ccccc6)c5Oc5cc(-c6ccccc6)cc3c54)cc2)cc1 ; c1ccc(N2c3ccccc3B3c4ncn(-c5ccccc5)c4Oc4cc(-n5c6ccccc6c6ccccc65)cc2c43)cc1 ; CC(C)(C)c1ccc(N2c3ccc(C(C)(C)C)cc3B3c4ncn(-c5ccccc5)c4Oc4cc(-n5c6ccccc6c6ccccc65)cc2c43)cc1]

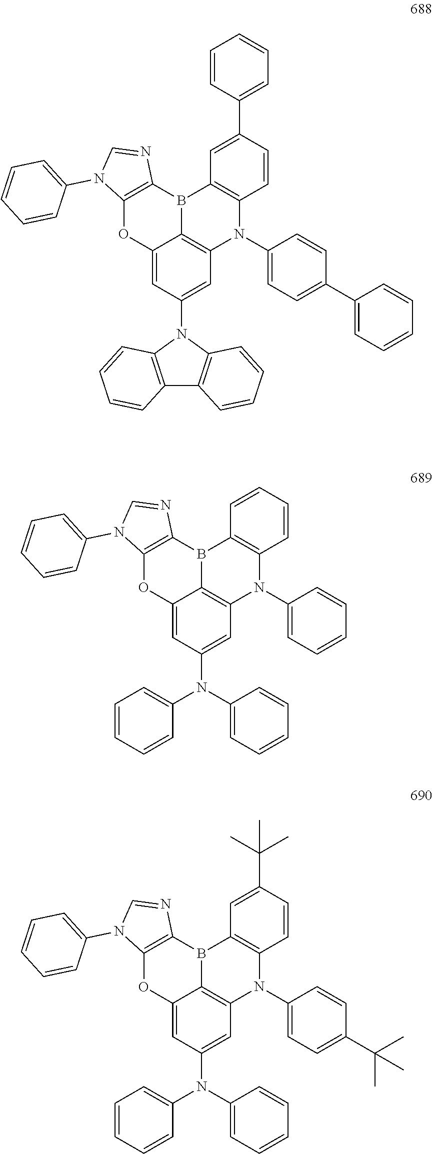 [SMILES: c1ccc(-c2ccc(N3c4ccc(-c5ccccc5)cc4B4c5ncn(-c6ccccc6)c5Oc5cc(-n6c7ccccc7c7ccccc76)cc3c54)cc2)cc1 ; c1ccc(N(c2ccccc2)c2cc3c4c(c2)N(c2ccccc2)c2ccccc2B4c2ncn(-c4ccccc4)c2O3)cc1 ; CC(C)(C)c1ccc(N2c3ccc(C(C)(C)C)cc3B3c4ncn(-c5ccccc5)c4Oc4cc(N(c5ccccc5)c5ccccc5)cc2c43)cc1]

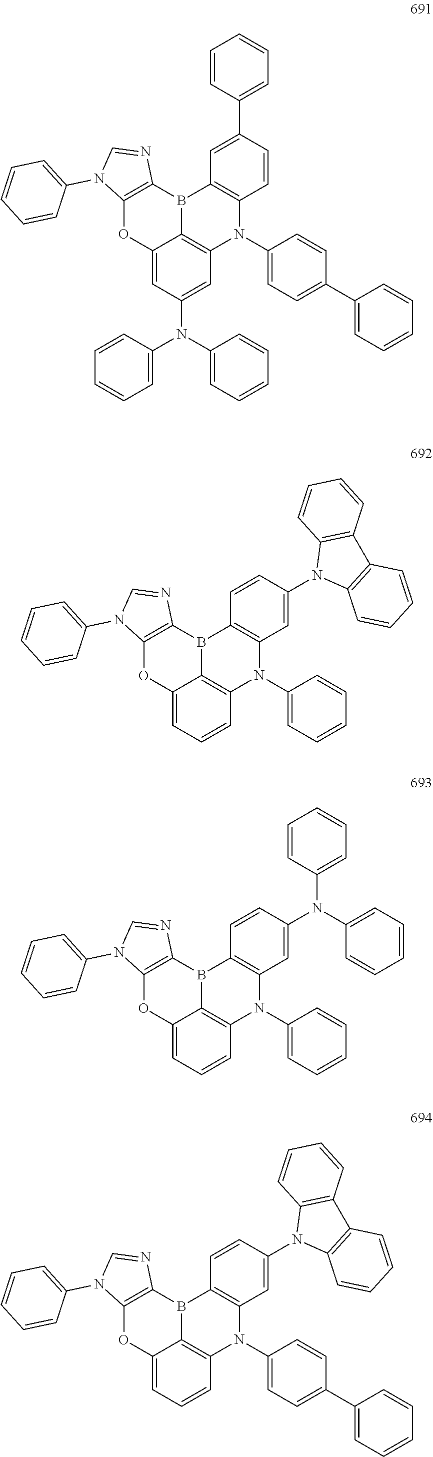 [SMILES: c1ccc(-c2ccc(N3c4ccc(-c5ccccc5)cc4B4c5ncn(-c6ccccc6)c5Oc5cc(N(c6ccccc6)c6ccccc6)cc3c54)cc2)cc1 ; c1ccc(N2c3cc(-n4c5ccccc5c5ccccc54)ccc3B3c4ncn(-c5ccccc5)c4Oc4cccc2c43)cc1 ; c1ccc(N(c2ccccc2)c2ccc3c(c2)N(c2ccccc2)c2cccc4c2B3c2ncn(-c3ccccc3)c2O4)cc1 ; c1ccc(-c2ccc(N3c4cc(-n5c6ccccc6c6ccccc65)ccc4B4c5ncn(-c6ccccc6)c5Oc5cccc3c54)cc2)cc1]

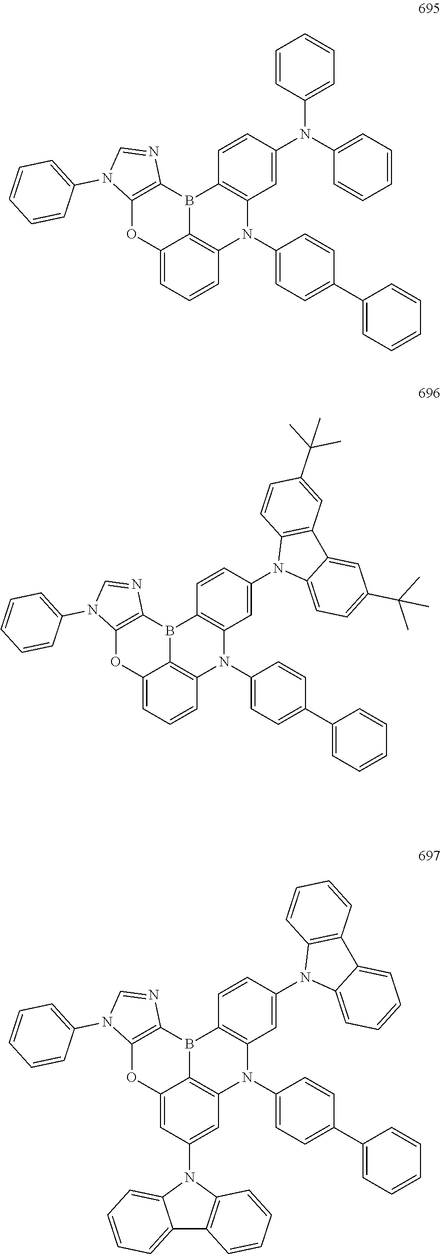 [SMILES: c1ccc(-c2ccc(N3c4cc(N(c5ccccc5)c5ccccc5)ccc4B4c5ncn(-c6ccccc6)c5Oc5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ncn(-c2ccccc2)c1O3 ; c1ccc(-c2ccc(N3c4cc(-n5c6ccccc6c6ccccc65)ccc4B4c5ncn(-c6ccccc6)c5Oc5cc(-n6c7ccccc7c7ccccc76)cc3c54)cc2)cc1]

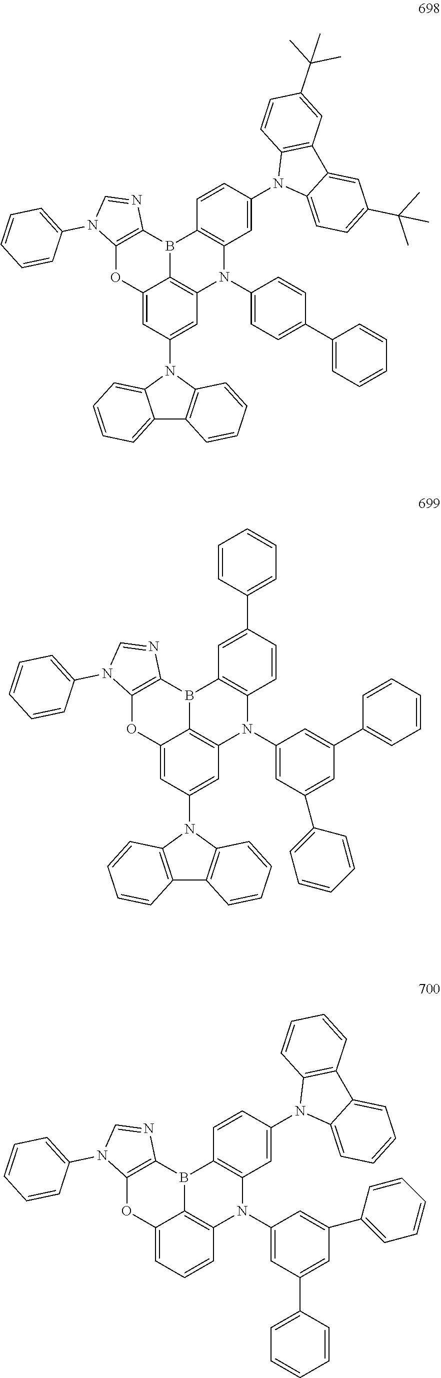 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ncn(-c2ccccc2)c1O3 ; c1ccc(-c2cc(-c3ccccc3)cc(N3c4ccc(-c5ccccc5)cc4B4c5ncn(-c6ccccc6)c5Oc5cc(-n6c7ccccc7c7ccccc76)cc3c54)c2)cc1 ; c1ccc(-c2cc(-c3ccccc3)cc(N3c4cc(-n5c6ccccc6c6ccccc65)ccc4B4c5ncn(-c6ccccc6)c5Oc5cccc3c54)c2)cc1]

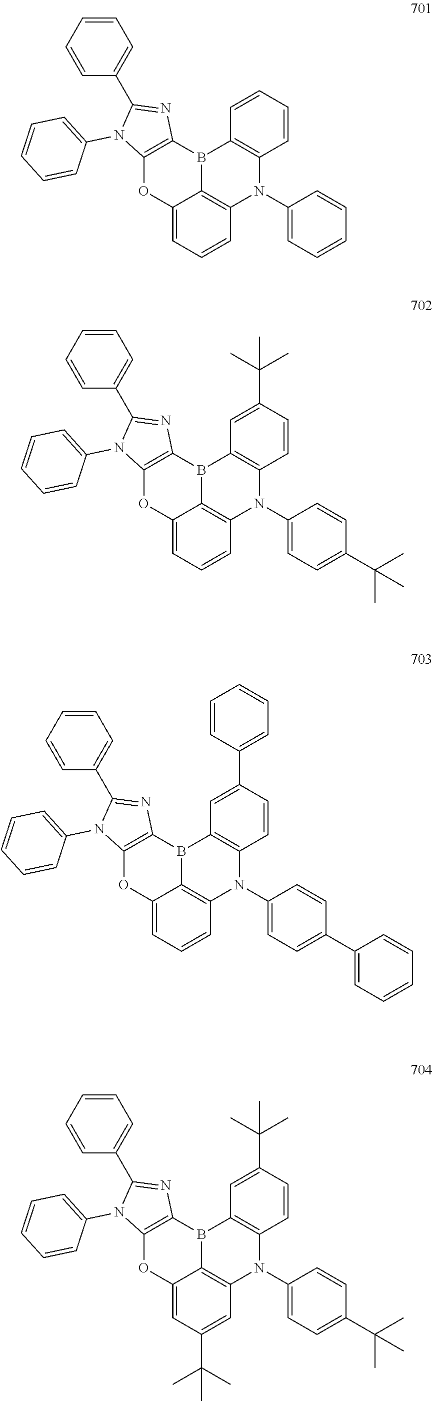 [SMILES: c1ccc(-c2nc3c(n2-c2ccccc2)Oc2cccc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4Oc4cccc2c43)cc1 ; c1ccc(-c2ccc(N3c4ccc(-c5ccccc5)cc4B4c5nc(-c6ccccc6)n(-c6ccccc6)c5Oc5cccc3c54)cc2)cc1 ; CC(C)(C)c1ccc(N2c3ccc(C(C)(C)C)cc3B3c4nc(-c5ccccc5)n(-c5ccccc5)c4Oc4cc(C(C)(C)C)cc2c43)cc1]

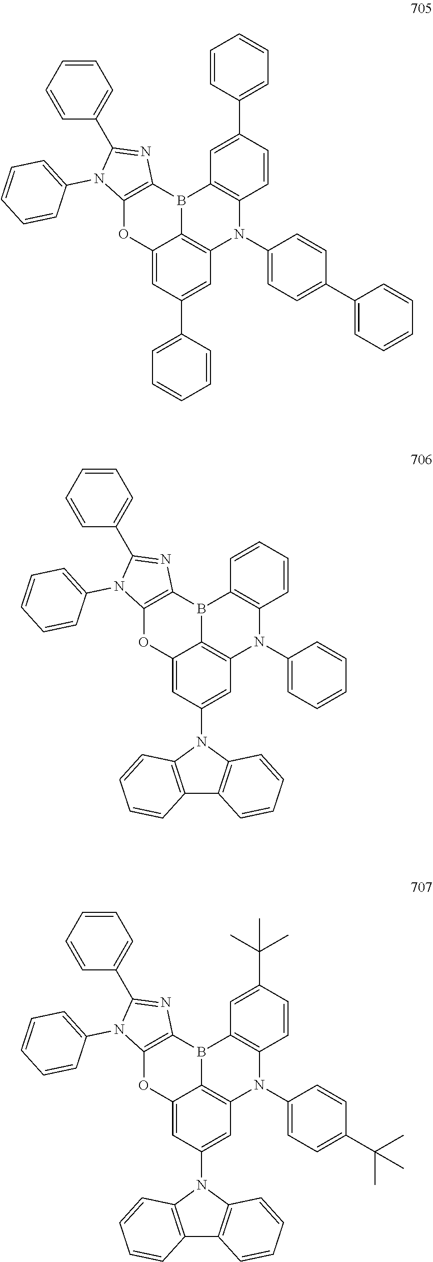 [SMILES: c1ccc(-c2ccc(N3c4ccc(-c5ccccc5)cc4B4c5nc(-c6ccccc6)n(-c6ccccc6)c5Oc5cc(-c6ccccc6)cc3c54)cc2)cc1 ; c1ccc(-c2nc3c(n2-c2ccccc2)Oc2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4Oc4cc(-n5c6ccccc6c6ccccc65)cc2c43)cc1]

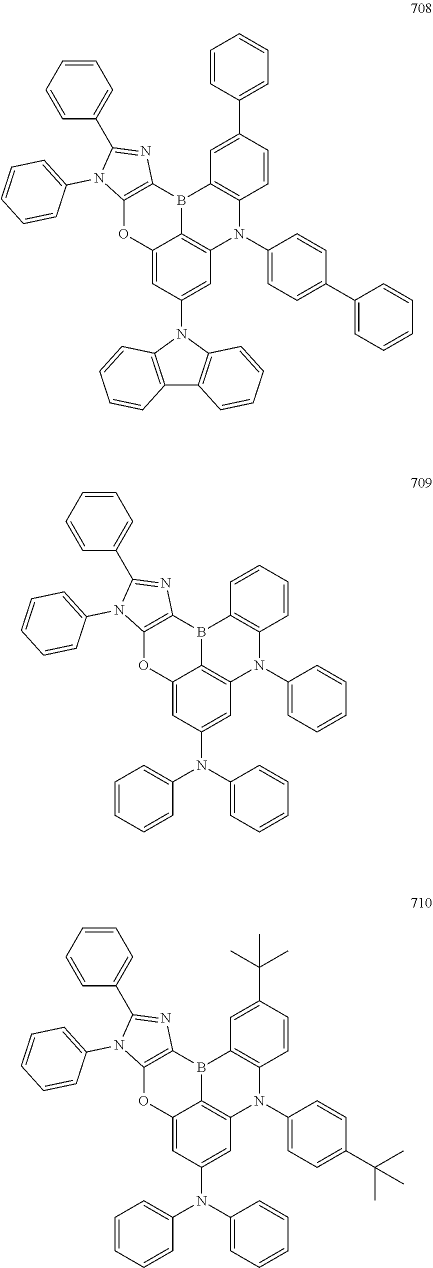 [SMILES: c1ccc(-c2ccc(N3c4ccc(-c5ccccc5)cc4B4c5nc(-c6ccccc6)n(-c6ccccc6)c5Oc5cc(-n6c7ccccc7c7ccccc76)cc3c54)cc2)cc1 ; c1ccc(-c2nc3c(n2-c2ccccc2)Oc2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4Oc4cc(N(c5ccccc5)c5ccccc5)cc2c43)cc1]

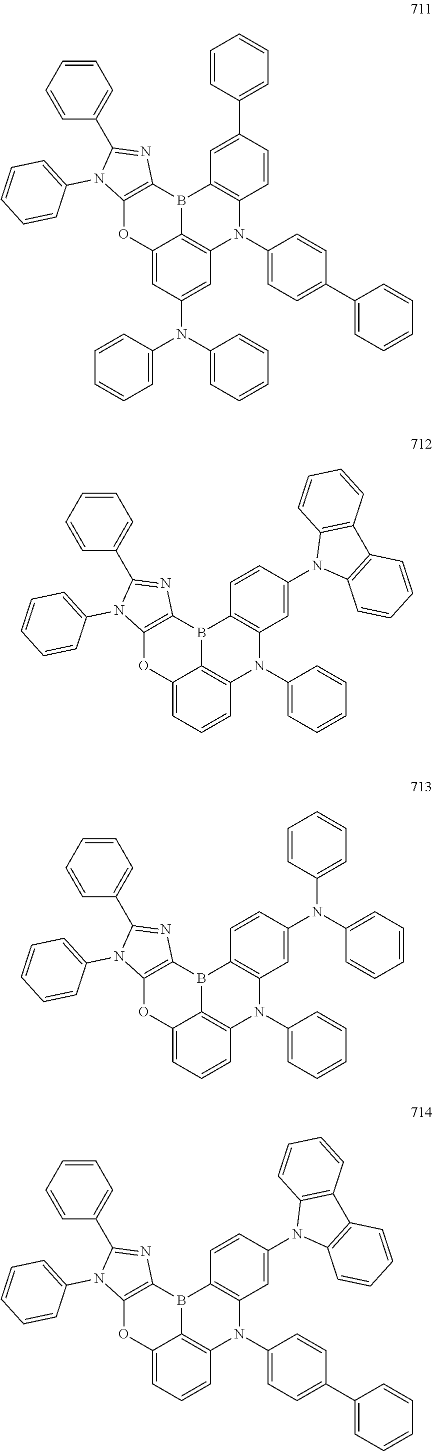 [SMILES: c1ccc(-c2ccc(N3c4ccc(-c5ccccc5)cc4B4c5nc(-c6ccccc6)n(-c6ccccc6)c5Oc5cc(N(c6ccccc6)c6ccccc6)cc3c54)cc2)cc1 ; c1ccc(-c2nc3c(n2-c2ccccc2)Oc2cccc4c2B3c2ccc(-n3c5ccccc5c5ccccc53)cc2N4c2ccccc2)cc1 ; c1ccc(-c2nc3c(n2-c2ccccc2)Oc2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5nc(-c6ccccc6)n(-c6ccccc6)c5Oc5cccc3c54)cc2)cc1]

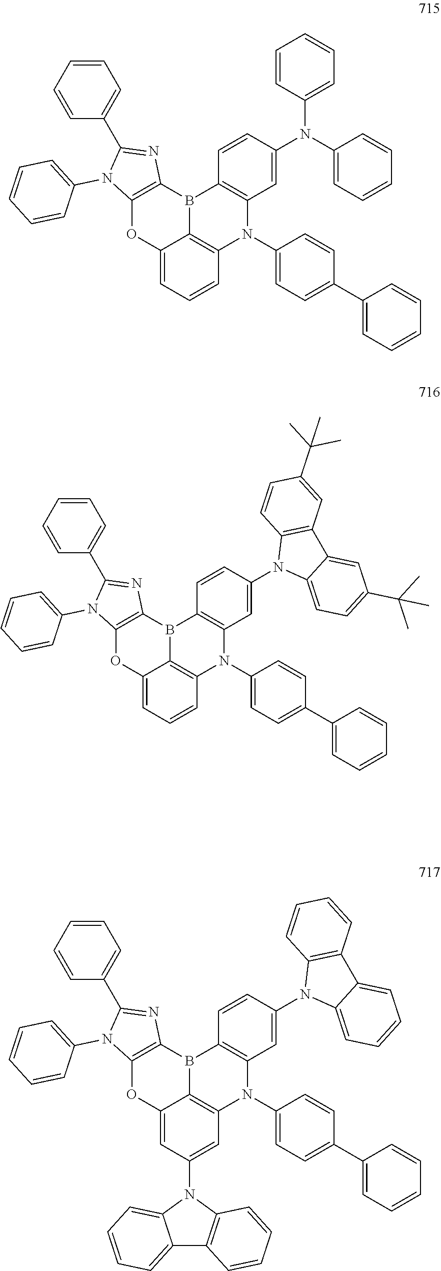 [SMILES: c1ccc(-c2ccc(N3c4cc(N(c5ccccc5)c5ccccc5)ccc4B4c5nc(-c6ccccc6)n(-c6ccccc6)c5Oc5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nc(-c2ccccc2)n(-c2ccccc2)c1O3 ; c1ccc(-c2ccc(N3c4cc(-n5c6ccccc6c6ccccc65)ccc4B4c5nc(-c6ccccc6)n(-c6ccccc6)c5Oc5cc(-n6c7ccccc7c7ccccc76)cc3c54)cc2)cc1]

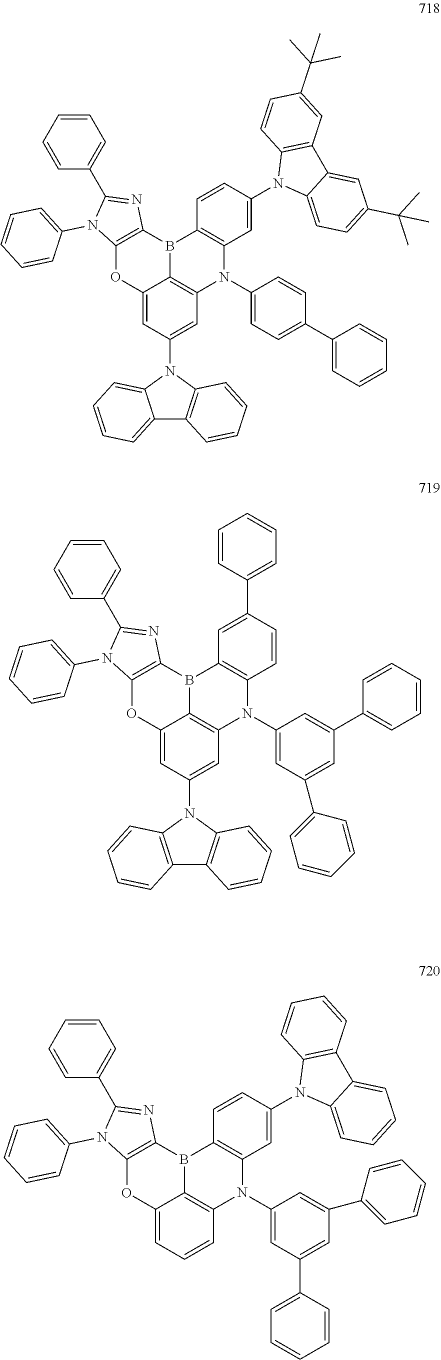 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nc(-c2ccccc2)n(-c2ccccc2)c1O3 ; c1ccc(-c2cc(-c3ccccc3)cc(N3c4ccc(-c5ccccc5)cc4B4c5nc(-c6ccccc6)n(-c6ccccc6)c5Oc5cc(-n6c7ccccc7c7ccccc76)cc3c54)c2)cc1 ; c1ccc(-c2cc(-c3ccccc3)cc(N3c4cc(-n5c6ccccc6c6ccccc65)ccc4B4c5nc(-c6ccccc6)n(-c6ccccc6)c5Oc5cccc3c54)c2)cc1]

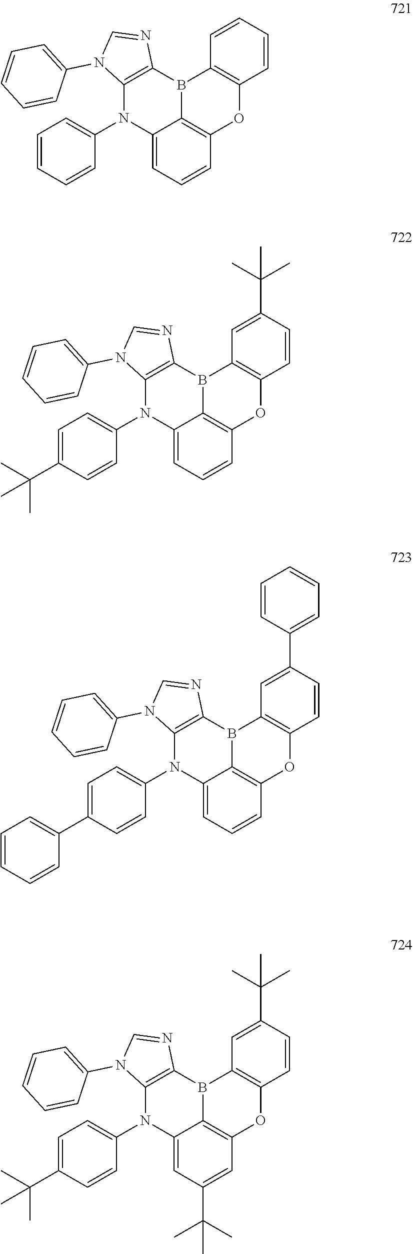

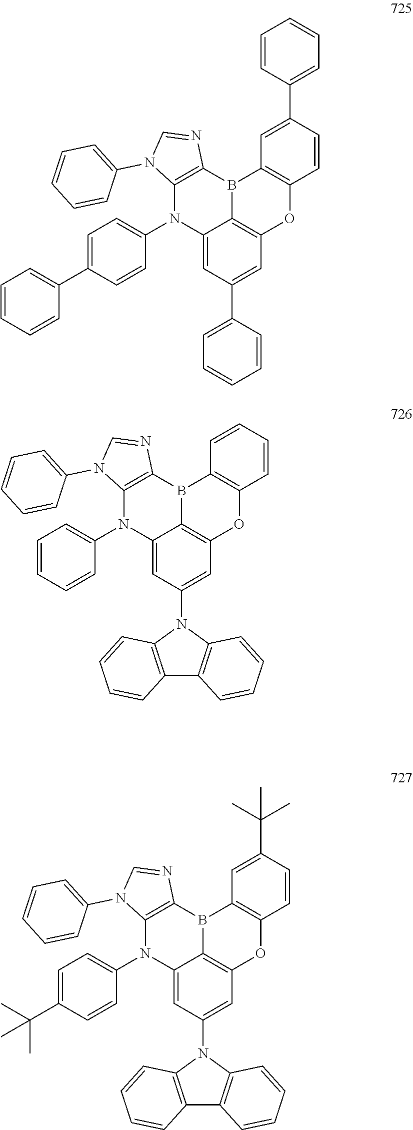

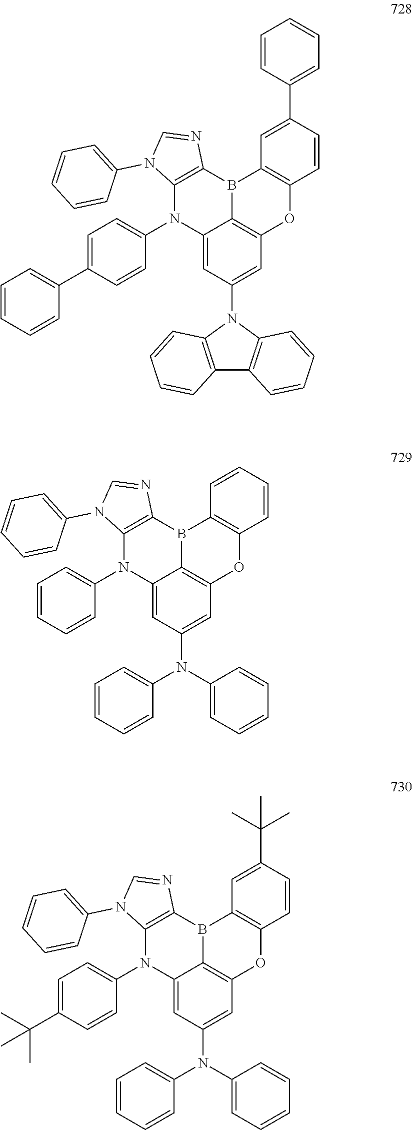 [SMILES: c1ccc(-c2ccc(N3c4cc(-n5c6ccccc6c6ccccc65)cc5c4B(c4cc(-c6ccccc6)ccc4O5)c4ncn(-c5ccccc5)c43)cc2)cc1 ; c1ccc(N(c2ccccc2)c2cc3c4c(c2)N(c2ccccc2)c2c(ncn2-c2ccccc2)B4c2ccccc2O3)cc1 ; CC(C)(C)c1ccc(N2c3cc(N(c4ccccc4)c4ccccc4)cc4c3B(c3cc(C(C)(C)C)ccc3O4)c3ncn(-c4ccccc4)c32)cc1]

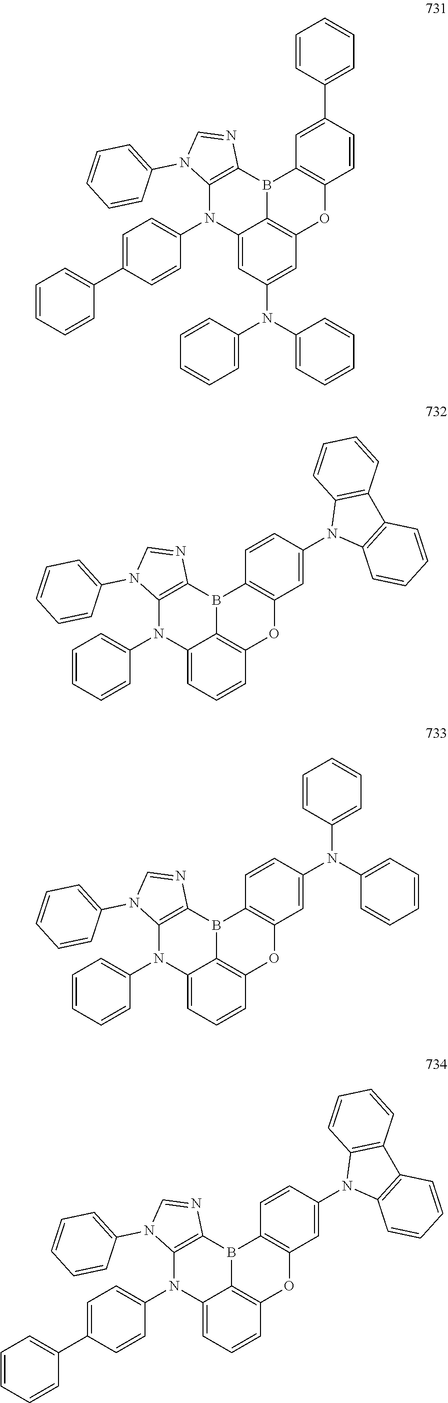 [SMILES: c1ccc(-c2ccc(N3c4cc(N(c5ccccc5)c5ccccc5)cc5c4B(c4cc(-c6ccccc6)ccc4O5)c4ncn(-c5ccccc5)c43)cc2)cc1 ; c1ccc(N2c3cccc4c3B(c3ccc(-n5c6ccccc6c6ccccc65)cc3O4)c3ncn(-c4ccccc4)c32)cc1 ; c1ccc(N(c2ccccc2)c2ccc3c(c2)Oc2cccc4c2B3c2ncn(-c3ccccc3)c2N4c2ccccc2)cc1 ; c1ccc(-c2ccc(N3c4cccc5c4B(c4ccc(-n6c7ccccc7c7ccccc76)cc4O5)c4ncn(-c5ccccc5)c43)cc2)cc1]

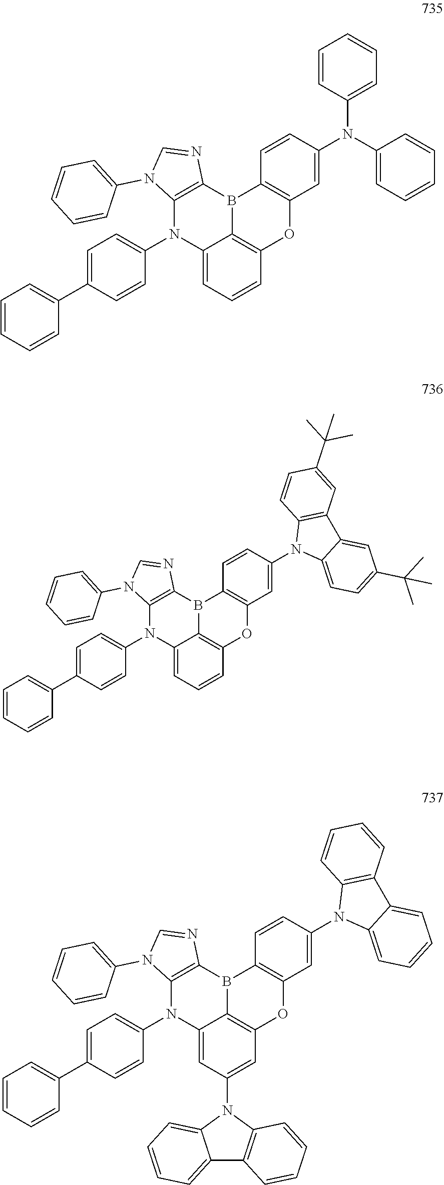 [SMILES: c1ccc(-c2ccc(N3c4cccc5c4B(c4ccc(N(c6ccccc6)c6ccccc6)cc4O5)c4ncn(-c5ccccc5)c43)cc2)cc1 ; CC(C)(C)c1ccc2c(c1)c1cc(C(C)(C)C)ccc1n2-c1ccc2c(c1)Oc1cccc3c1B2c1ncn(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4ncn(-c5ccccc5)c43)cc2)cc1]

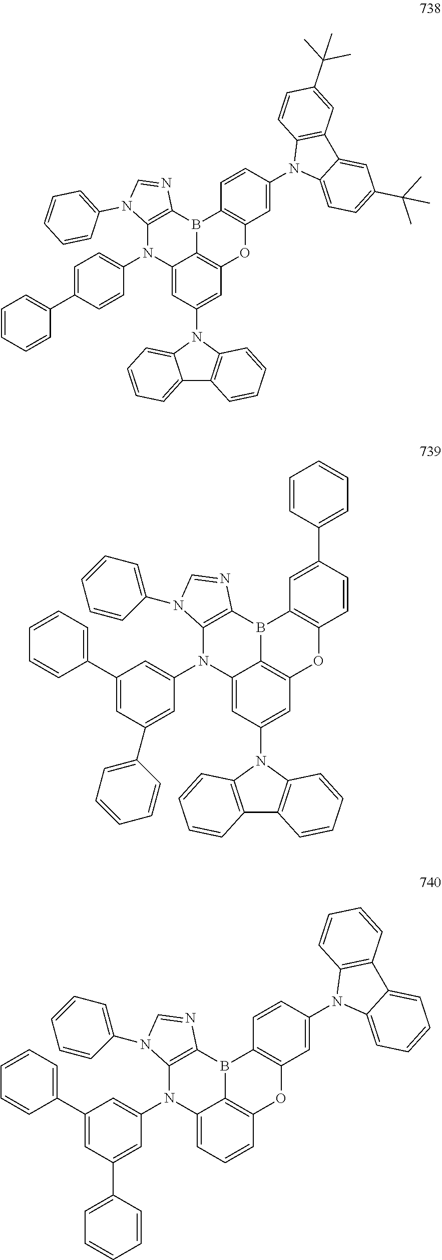 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1ncn(-c2ccccc2)c1N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4ncn(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4ncn(-c5ccccc5)c43)c2)cc1]

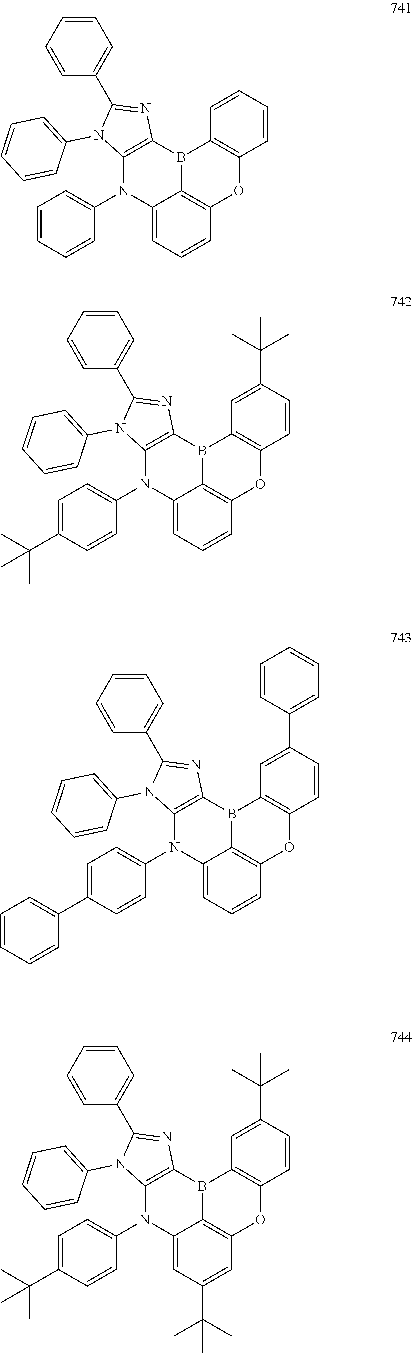

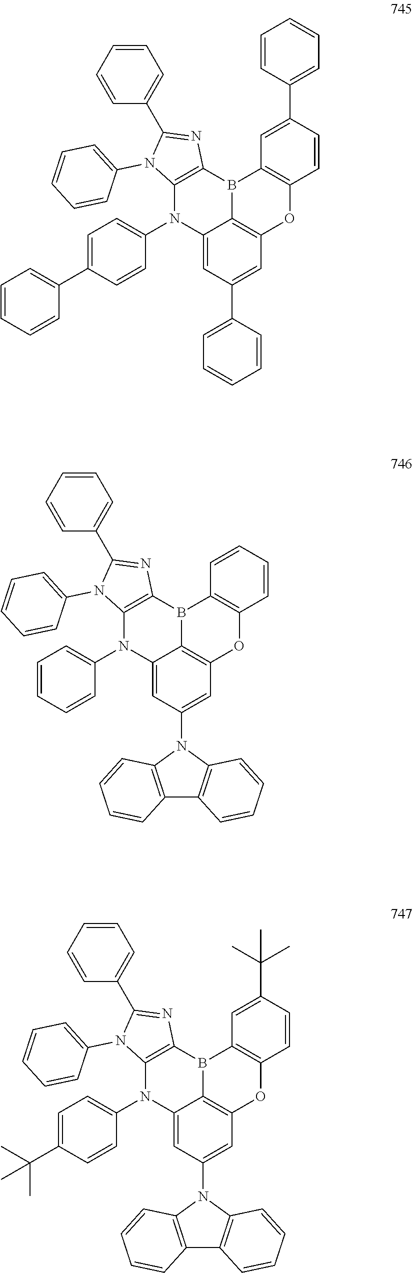 [SMILES: c1ccc(-c2ccc(N3c4cc(-c5ccccc5)cc5c4B(c4cc(-c6ccccc6)ccc4O5)c4nc(-c5ccccc5)n(-c5ccccc5)c43)cc2)cc1 ; c1ccc(-c2nc3c(n2-c2ccccc2)N(c2ccccc2)c2cc(-n4c5ccccc5c5ccccc54)cc4c2B3c2ccccc2O4)cc1 ; CC(C)(C)c1ccc(N2c3cc(-n4c5ccccc5c5ccccc54)cc4c3B(c3cc(C(C)(C)C)ccc3O4)c3nc(-c4ccccc4)n(-c4ccccc4)c32)cc1]

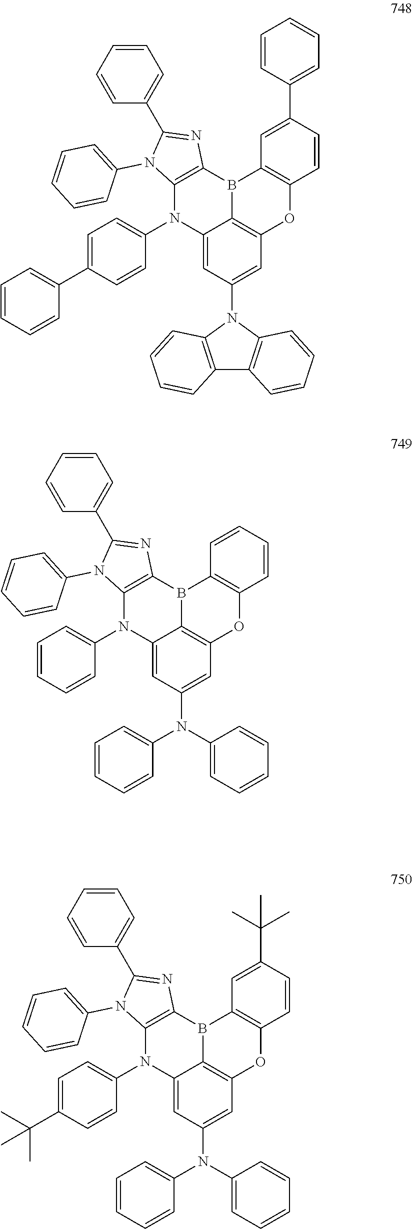 [SMILES: c1ccc(-c2ccc(N3c4cc(-n5c6ccccc6c6ccccc65)cc5c4B(c4cc(-c6ccccc6)ccc4O5)c4nc(-c5ccccc5)n(-c5ccccc5)c43)cc2)cc1 ; c1ccc(-c2nc3c(n2-c2ccccc2)N(c2ccccc2)c2cc(N(c4ccccc4)c4ccccc4)cc4c2B3c2ccccc2O4)cc1 ; CC(C)(C)c1ccc(N2c3cc(N(c4ccccc4)c4ccccc4)cc4c3B(c3cc(C(C)(C)C)ccc3O4)c3nc(-c4ccccc4)n(-c4ccccc4)c32)cc1]

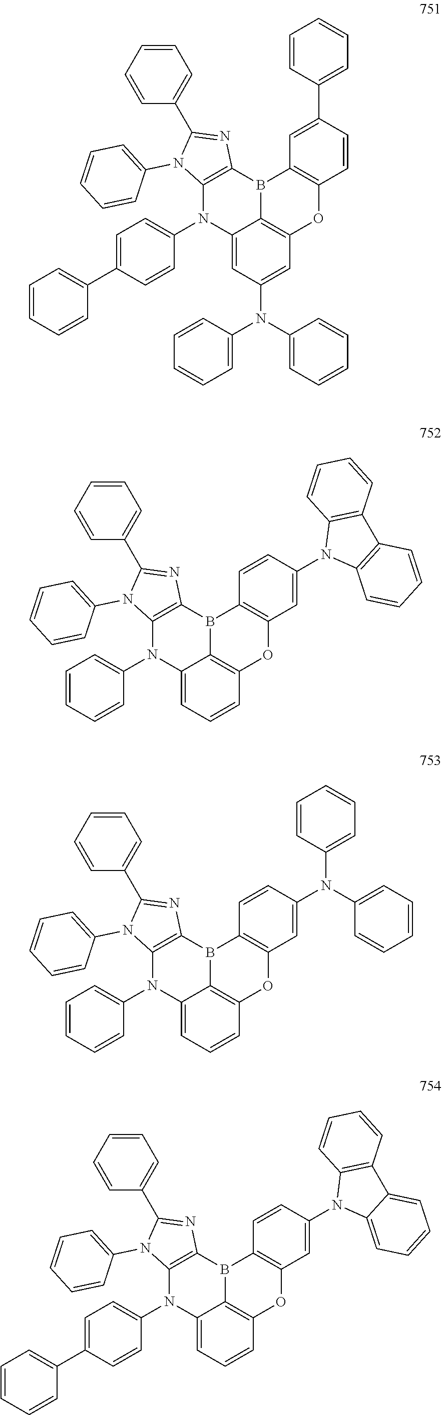 [SMILES: c1ccc(-c2ccc(N3c4cc(N(c5ccccc5)c5ccccc5)cc5c4B(c4cc(-c6ccccc6)ccc4O5)c4nc(-c5ccccc5)n(-c5ccccc5)c43)cc2)cc1 ; c1ccc(-c2nc3c(n2-c2ccccc2)N(c2ccccc2)c2cccc4c2B3c2ccc(-n3c5ccccc5c5ccccc53)cc2O4)cc1 ; c1ccc(-c2nc3c(n2-c2ccccc2)N(c2ccccc2)c2cccc4c2B3c2ccc(N(c3ccccc3)c3ccccc3)cc2O4)cc1 ; c1ccc(-c2ccc(N3c4cccc5c4B(c4ccc(-n6c7ccccc7c7ccccc76)cc4O5)c4nc(-c5ccccc5)n(-c5ccccc5)c43)cc2)cc1]

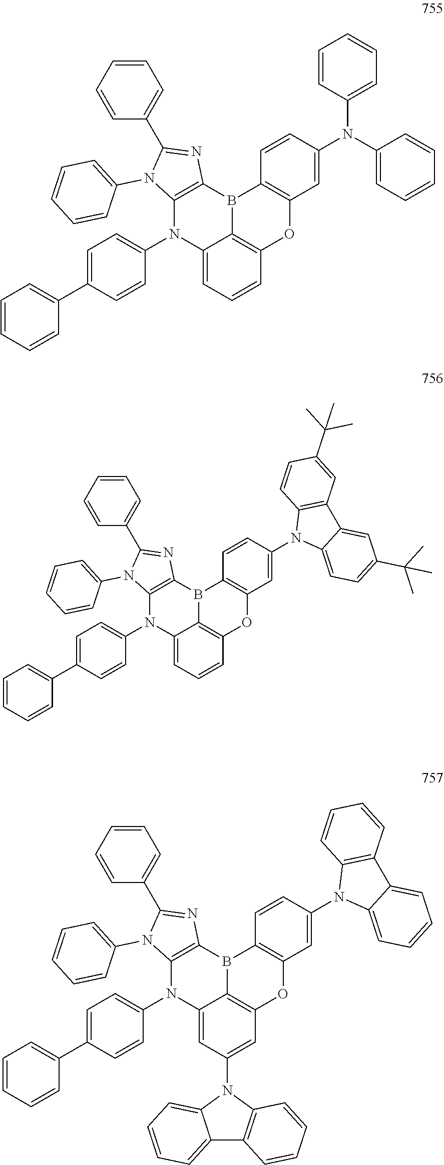 [SMILES: c1ccc(-c2ccc(N3c4cccc5c4B(c4ccc(N(c6ccccc6)c6ccccc6)cc4O5)c4nc(-c5ccccc5)n(-c5ccccc5)c43)cc2)cc1 ; CC(C)(C)c1ccc2c(c1)c1cc(C(C)(C)C)ccc1n2-c1ccc2c(c1)Oc1cccc3c1B2c1nc(-c2ccccc2)n(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4nc(-c5ccccc5)n(-c5ccccc5)c43)cc2)cc1]

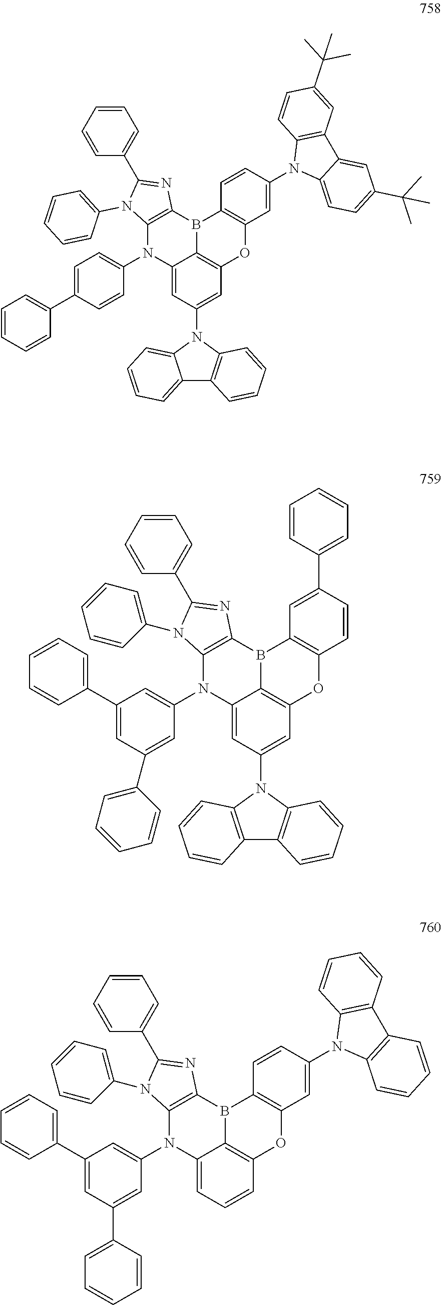 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1nc(-c2ccccc2)n(-c2ccccc2)c1N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4nc(-c5ccccc5)n(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4nc(-c5ccccc5)n(-c5ccccc5)c43)c2)cc1]

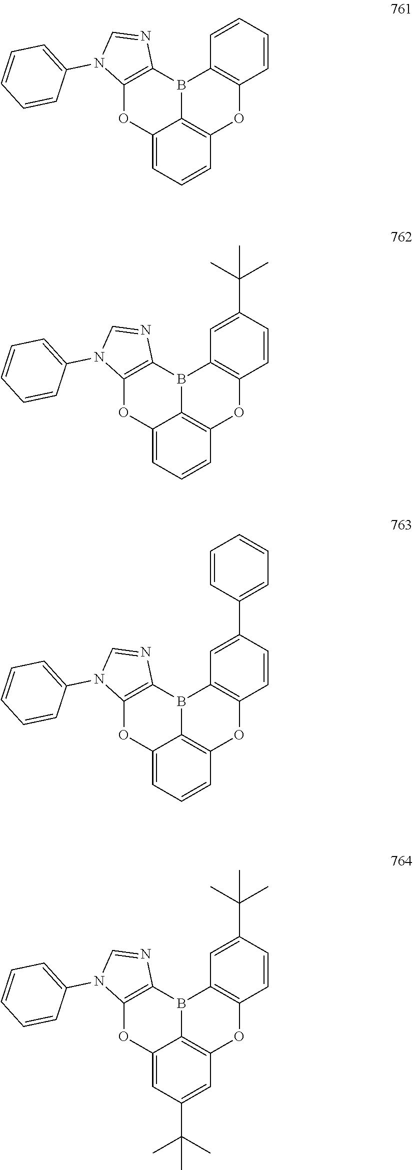 [SMILES: c1ccc(-n2cnc3c2Oc2cccc4c2B3c2ccccc2O4)cc1 ; CC(C)(C)c1ccc2c(c1)B1c3ncn(-c4ccccc4)c3Oc3cccc(c31)O2 ; c1ccc(-c2ccc3c(c2)B2c4ncn(-c5ccccc5)c4Oc4cccc(c42)O3)cc1 ; CC(C)(C)c1cc2c3c(c1)Oc1c(ncn1-c1ccccc1)B3c1cc(C(C)(C)C)ccc1O2]

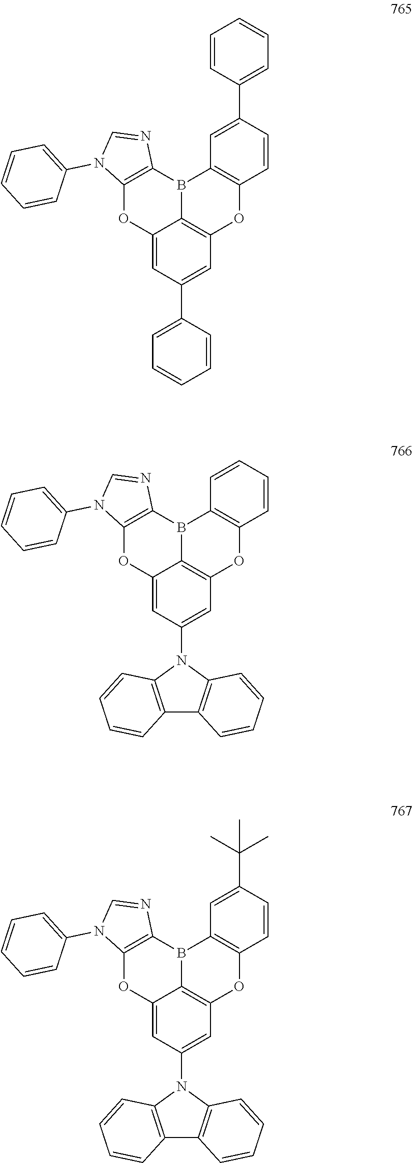 [SMILES: c1ccc(-c2cc3c4c(c2)Oc2c(ncn2-c2ccccc2)B4c2cc(-c4ccccc4)ccc2O3)cc1 ; c1ccc(-n2cnc3c2Oc2cc(-n4c5ccccc5c5ccccc54)cc4c2B3c2ccccc2O4)cc1 ; CC(C)(C)c1ccc2c(c1)B1c3ncn(-c4ccccc4)c3Oc3cc(-n4c5ccccc5c5ccccc54)cc(c31)O2]

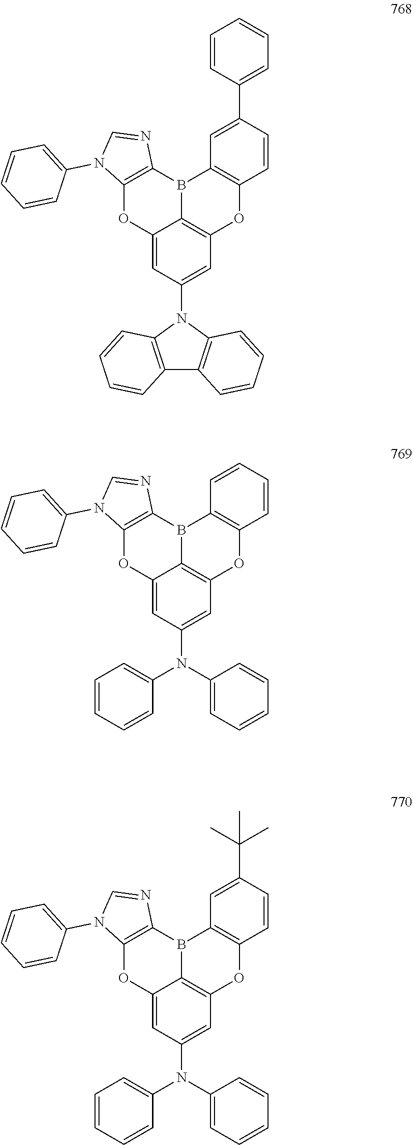 [SMILES: c1ccc(-c2ccc3c(c2)B2c4ncn(-c5ccccc5)c4Oc4cc(-n5c6ccccc6c6ccccc65)cc(c42)O3)cc1 ; c1ccc(N(c2ccccc2)c2cc3c4c(c2)Oc2c(ncn2-c2ccccc2)B4c2ccccc2O3)cc1 ; CC(C)(C)c1ccc2c(c1)B1c3ncn(-c4ccccc4)c3Oc3cc(N(c4ccccc4)c4ccccc4)cc(c31)O2]

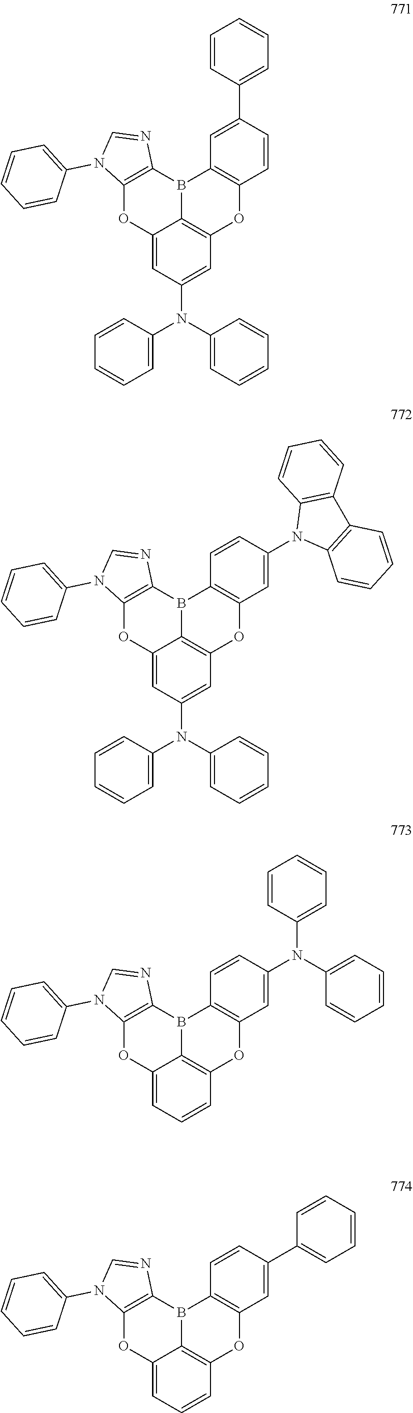 [SMILES: c1ccc(-c2ccc3c(c2)B2c4ncn(-c5ccccc5)c4Oc4cc(N(c5ccccc5)c5ccccc5)cc(c42)O3)cc1 ; c1ccc(N(c2ccccc2)c2cc3c4c(c2)Oc2c(ncn2-c2ccccc2)B4c2ccc(-n4c5ccccc5c5ccccc54)cc2O3)cc1 ; c1ccc(N(c2ccccc2)c2ccc3c(c2)Oc2cccc4c2B3c2ncn(-c3ccccc3)c2O4)cc1 ; c1ccc(-c2ccc3c(c2)Oc2cccc4c2B3c2ncn(-c3ccccc3)c2O4)cc1]

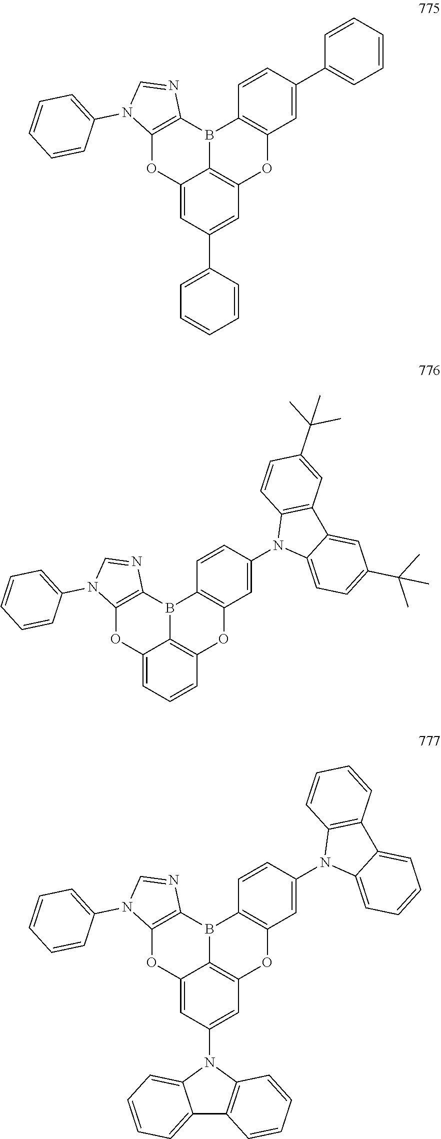 [SMILES: c1ccc(-c2ccc3c(c2)Oc2cc(-c4ccccc4)cc4c2B3c2ncn(-c3ccccc3)c2O4)cc1 ; CC(C)(C)c1ccc2c(c1)c1cc(C(C)(C)C)ccc1n2-c1ccc2c(c1)Oc1cccc3c1B2c1ncn(-c2ccccc2)c1O3 ; c1ccc(-n2cnc3c2Oc2cc(-n4c5ccccc5c5ccccc54)cc4c2B3c2ccc(-n3c5ccccc5c5ccccc53)cc2O4)cc1]

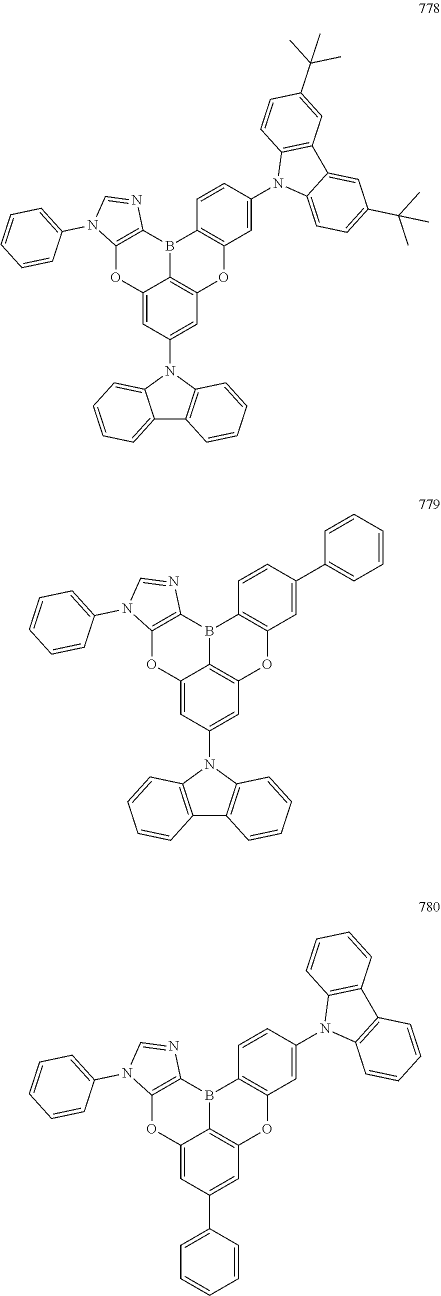 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1ncn(-c2ccccc2)c1O3 ; c1ccc(-c2ccc3c(c2)Oc2cc(-n4c5ccccc5c5ccccc54)cc4c2B3c2ncn(-c3ccccc3)c2O4)cc1 ; c1ccc(-c2cc3c4c(c2)Oc2c(ncn2-c2ccccc2)B4c2ccc(-n4c5ccccc5c5ccccc54)cc2O3)cc1]

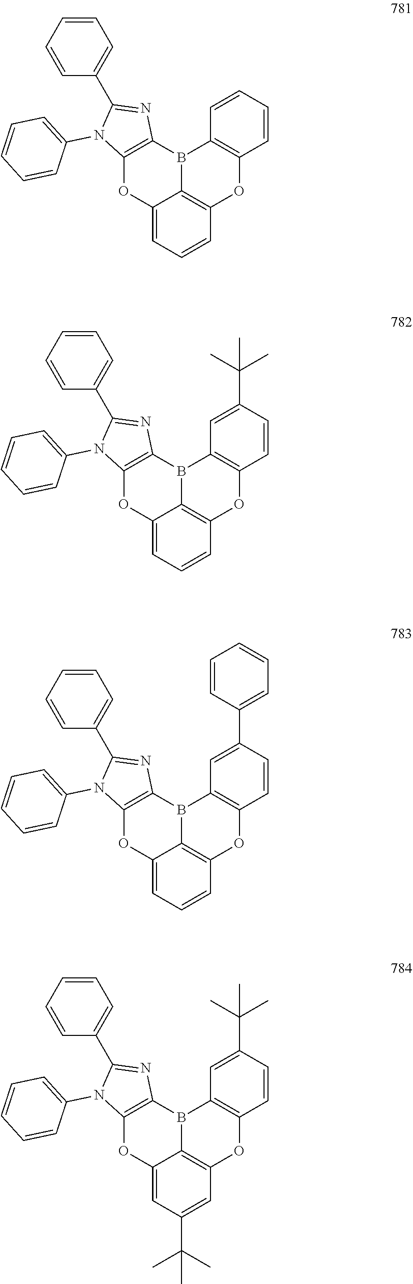 [SMILES: c1ccc(-c2nc3c(n2-c2ccccc2)Oc2cccc4c2B3c2ccccc2O4)cc1 ; CC(C)(C)c1ccc2c(c1)B1c3nc(-c4ccccc4)n(-c4ccccc4)c3Oc3cccc(c31)O2 ; c1ccc(-c2ccc3c(c2)B2c4nc(-c5ccccc5)n(-c5ccccc5)c4Oc4cccc(c42)O3)cc1 ; CC(C)(C)c1cc2c3c(c1)Oc1c(nc(-c4ccccc4)n1-c1ccccc1)B3c1cc(C(C)(C)C)ccc1O2]

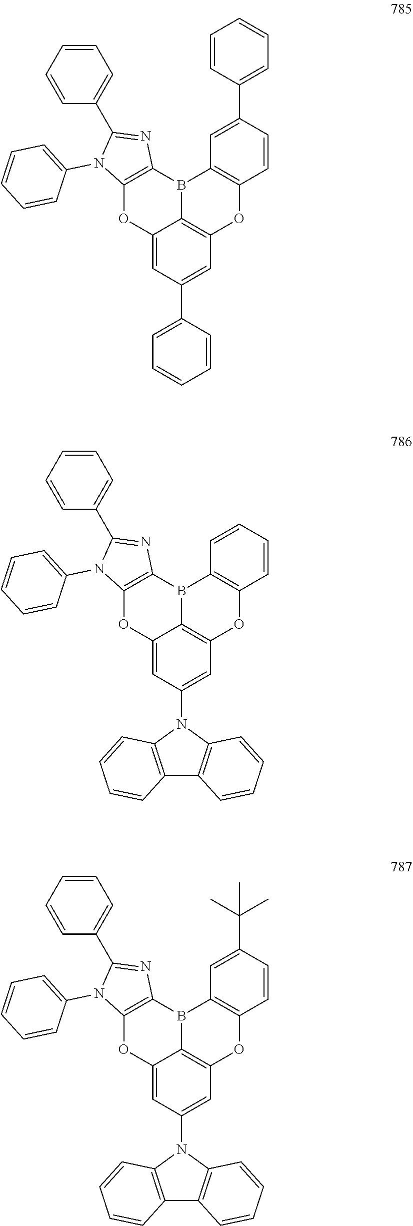 [SMILES: c1ccc(-c2cc3c4c(c2)Oc2c(nc(-c5ccccc5)n2-c2ccccc2)B4c2cc(-c4ccccc4)ccc2O3)cc1 ; c1ccc(-c2nc3c(n2-c2ccccc2)Oc2cc(-n4c5ccccc5c5ccccc54)cc4c2B3c2ccccc2O4)cc1 ; CC(C)(C)c1ccc2c(c1)B1c3nc(-c4ccccc4)n(-c4ccccc4)c3Oc3cc(-n4c5ccccc5c5ccccc54)cc(c31)O2]

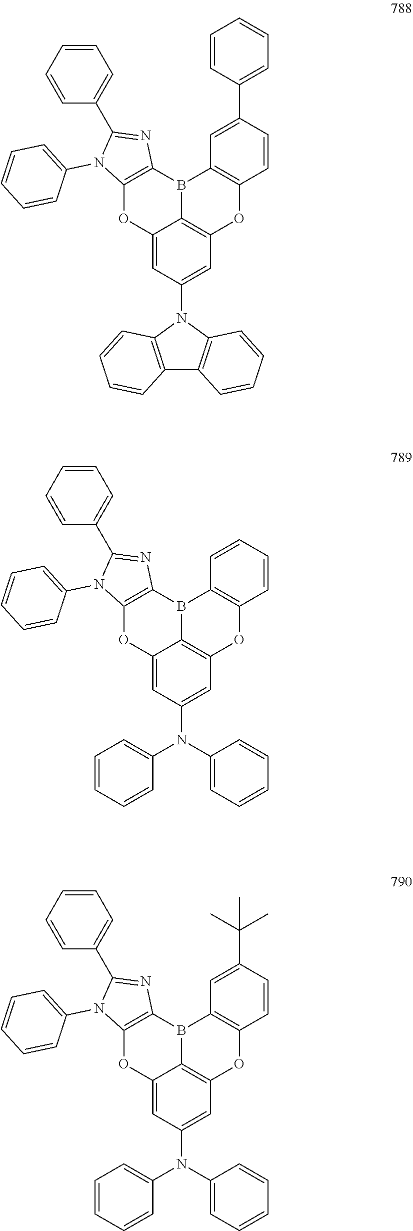 [SMILES: c1ccc(-c2ccc3c(c2)B2c4nc(-c5ccccc5)n(-c5ccccc5)c4Oc4cc(-n5c6ccccc6c6ccccc65)cc(c42)O3)cc1 ; c1ccc(-c2nc3c(n2-c2ccccc2)Oc2cc(N(c4ccccc4)c4ccccc4)cc4c2B3c2ccccc2O4)cc1 ; CC(C)(C)c1ccc2c(c1)B1c3nc(-c4ccccc4)n(-c4ccccc4)c3Oc3cc(N(c4ccccc4)c4ccccc4)cc(c31)O2]

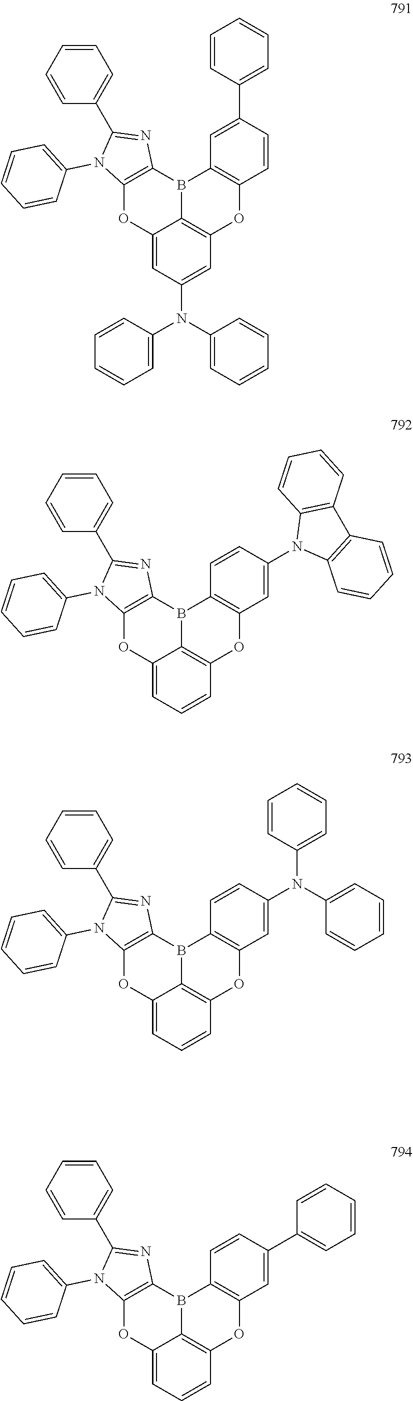 [SMILES: c1ccc(-c2ccc3c(c2)B2c4nc(-c5ccccc5)n(-c5ccccc5)c4Oc4cc(N(c5ccccc5)c5ccccc5)cc(c42)O3)cc1 ; c1ccc(-c2nc3c(n2-c2ccccc2)Oc2cccc4c2B3c2ccc(-n3c5ccccc5c5ccccc53)cc2O4)cc1 ; c1ccc(-c2nc3c(n2-c2ccccc2)Oc2cccc4c2B3c2ccc(N(c3ccccc3)c3ccccc3)cc2O4)cc1 ; c1ccc(-c2ccc3c(c2)Oc2cccc4c2B3c2nc(-c3ccccc3)n(-c3ccccc3)c2O4)cc1]

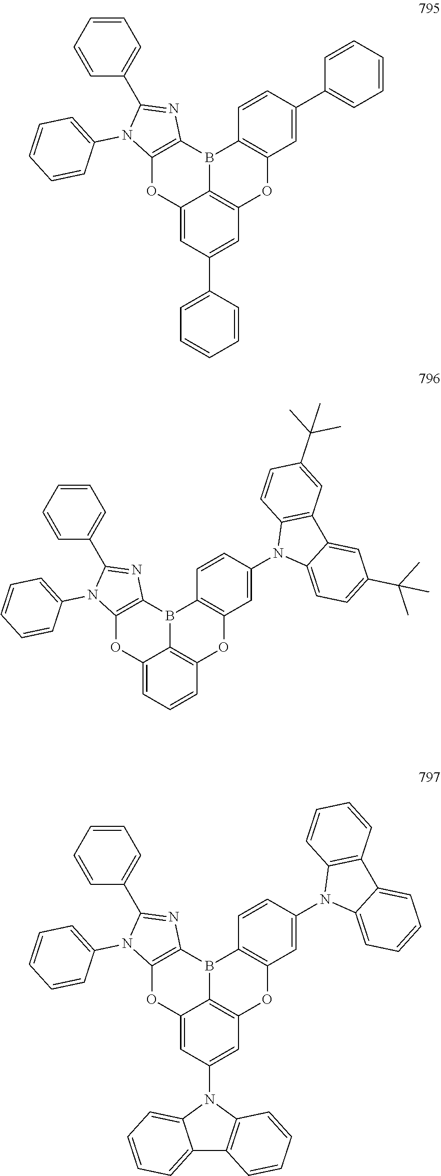 [SMILES: c1ccc(-c2ccc3c(c2)Oc2cc(-c4ccccc4)cc4c2B3c2nc(-c3ccccc3)n(-c3ccccc3)c2O4)cc1 ; CC(C)(C)c1ccc2c(c1)c1cc(C(C)(C)C)ccc1n2-c1ccc2c(c1)Oc1cccc3c1B2c1nc(-c2ccccc2)n(-c2ccccc2)c1O3 ; c1ccc(-c2nc3c(n2-c2ccccc2)Oc2cc(-n4c5ccccc5c5ccccc54)cc4c2B3c2ccc(-n3c5ccccc5c5ccccc53)cc2O4)cc1]

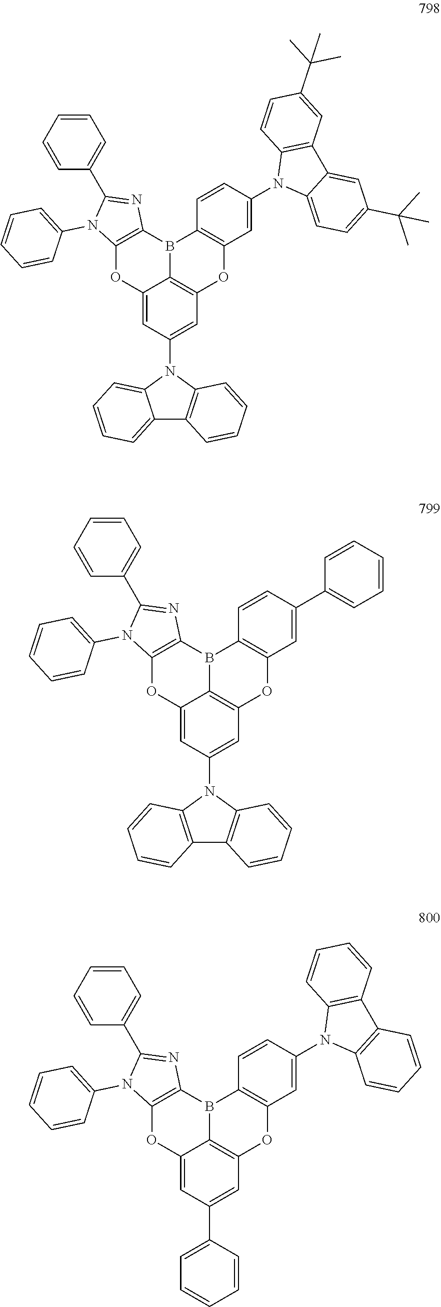 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1nc(-c2ccccc2)n(-c2ccccc2)c1O3 ; c1ccc(-c2ccc3c(c2)Oc2cc(-n4c5ccccc5c5ccccc54)cc4c2B3c2nc(-c3ccccc3)n(-c3ccccc3)c2O4)cc1 ; c1ccc(-c2cc3c4c(c2)Oc2c(nc(-c5ccccc5)n2-c2ccccc2)B4c2ccc(-n4c5ccccc5c5ccccc54)cc2O3)cc1]

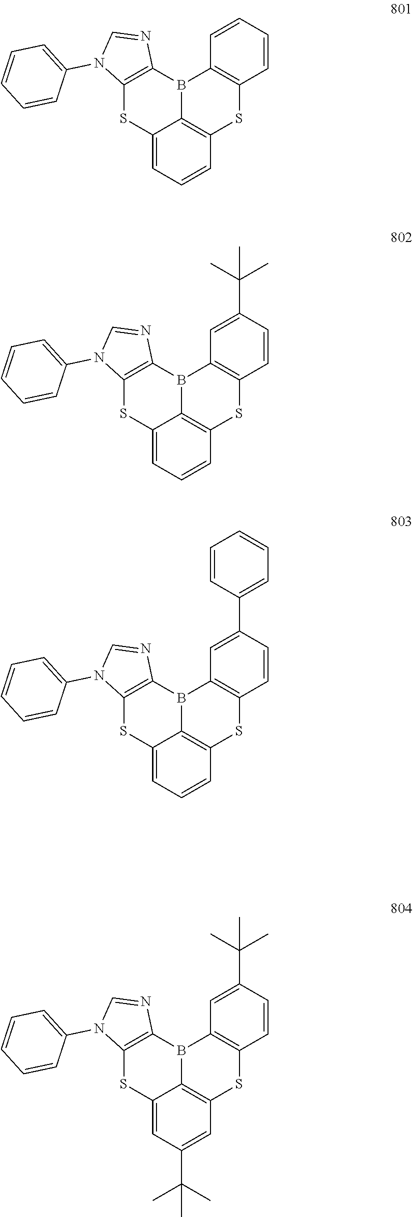 [SMILES: c1ccc(-n2cnc3c2Sc2cccc4c2B3c2ccccc2S4)cc1 ; CC(C)(C)c1ccc2c(c1)B1c3ncn(-c4ccccc4)c3Sc3cccc(c31)S2 ; c1ccc(-c2ccc3c(c2)B2c4ncn(-c5ccccc5)c4Sc4cccc(c42)S3)cc1 ; CC(C)(C)c1cc2c3c(c1)Sc1c(ncn1-c1ccccc1)B3c1cc(C(C)(C)C)ccc1S2]

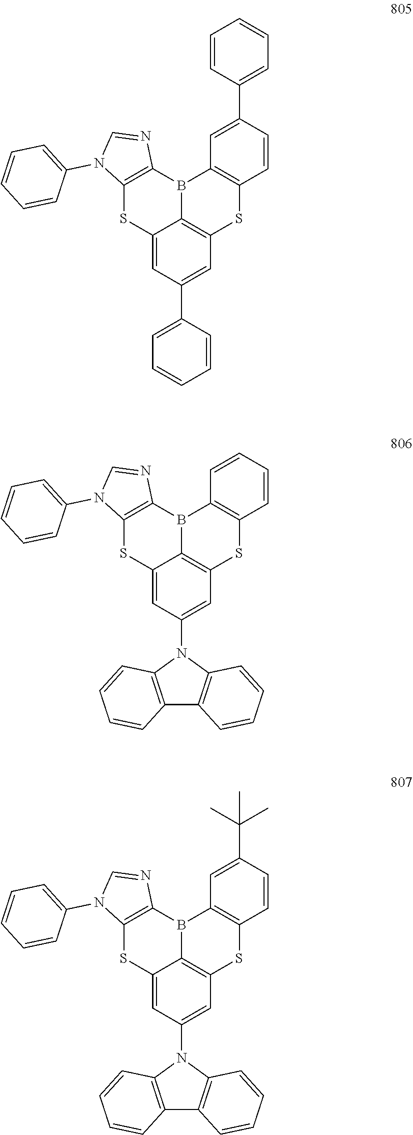 [SMILES: c1ccc(-c2cc3c4c(c2)Sc2c(ncn2-c2ccccc2)B4c2cc(-c4ccccc4)ccc2S3)cc1 ; c1ccc(-n2cnc3c2Sc2cc(-n4c5ccccc5c5ccccc54)cc4c2B3c2ccccc2S4)cc1 ; CC(C)(C)c1ccc2c(c1)B1c3ncn(-c4ccccc4)c3Sc3cc(-n4c5ccccc5c5ccccc54)cc(c31)S2]

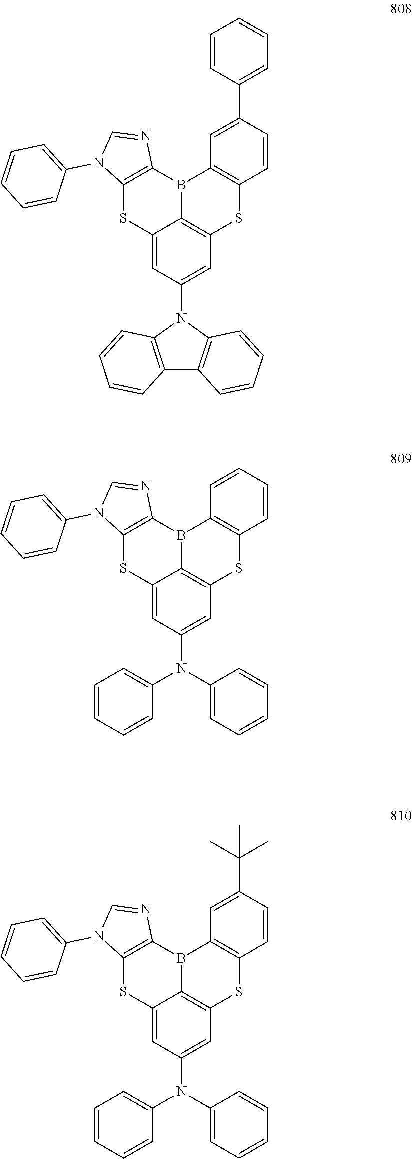 [SMILES: c1ccc(-c2ccc3c(c2)B2c4ncn(-c5ccccc5)c4Sc4cc(-n5c6ccccc6c6ccccc65)cc(c42)S3)cc1 ; c1ccc(N(c2ccccc2)c2cc3c4c(c2)Sc2c(ncn2-c2ccccc2)B4c2ccccc2S3)cc1 ; CC(C)(C)c1ccc2c(c1)B1c3ncn(-c4ccccc4)c3Sc3cc(N(c4ccccc4)c4ccccc4)cc(c31)S2]

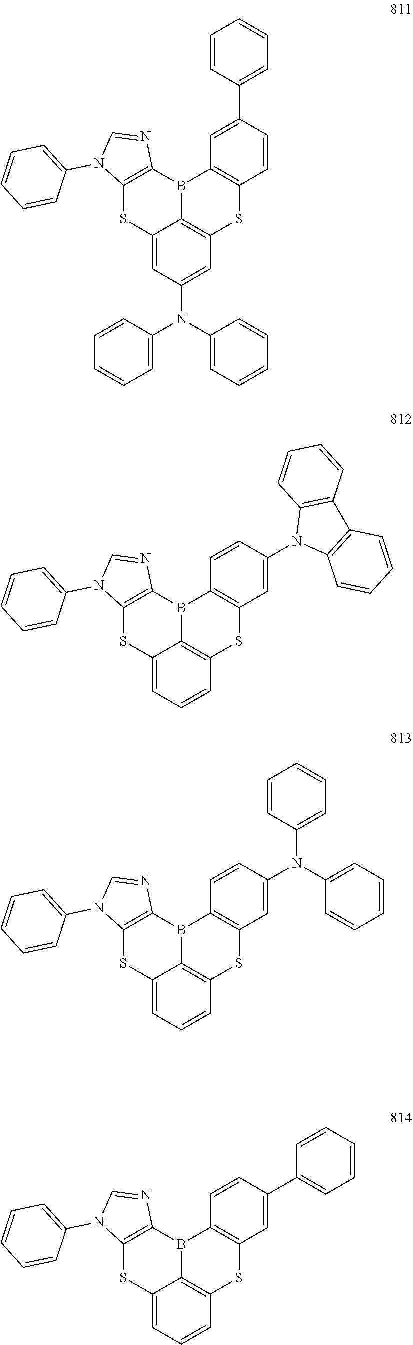 [SMILES: c1ccc(-c2ccc3c(c2)B2c4ncn(-c5ccccc5)c4Sc4cc(N(c5ccccc5)c5ccccc5)cc(c42)S3)cc1 ; c1ccc(-n2cnc3c2Sc2cccc4c2B3c2ccc(-n3c5ccccc5c5ccccc53)cc2S4)cc1 ; c1ccc(N(c2ccccc2)c2ccc3c(c2)Sc2cccc4c2B3c2ncn(-c3ccccc3)c2S4)cc1 ; c1ccc(-c2ccc3c(c2)Sc2cccc4c2B3c2ncn(-c3ccccc3)c2S4)cc1]

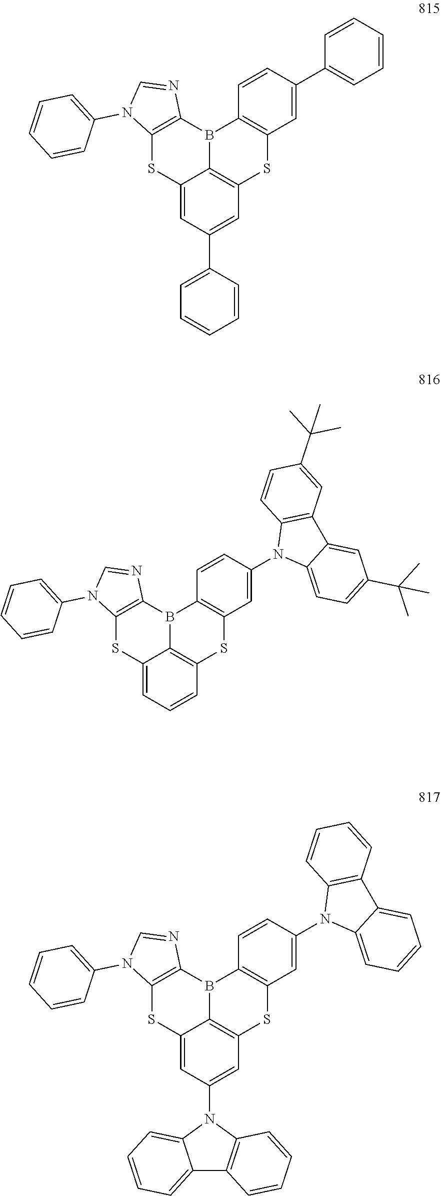 [SMILES: c1ccc(-c2ccc3c(c2)Sc2cc(-c4ccccc4)cc4c2B3c2ncn(-c3ccccc3)c2S4)cc1 ; CC(C)(C)c1ccc2c(c1)c1cc(C(C)(C)C)ccc1n2-c1ccc2c(c1)Sc1cccc3c1B2c1ncn(-c2ccccc2)c1S3 ; c1ccc(-n2cnc3c2Sc2cc(-n4c5ccccc5c5ccccc54)cc4c2B3c2ccc(-n3c5ccccc5c5ccccc53)cc2S4)cc1]

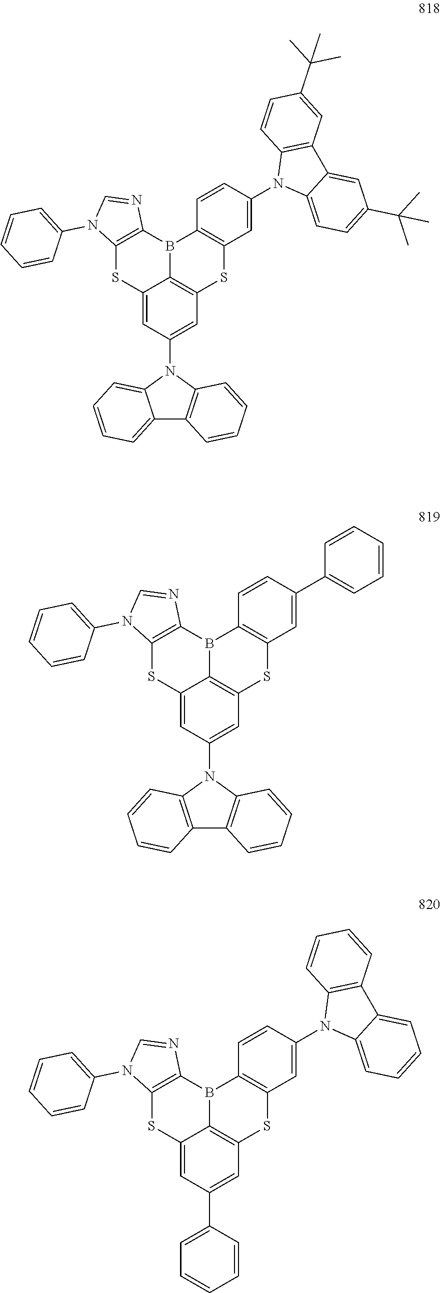 [SMILES: CC(C)(C)c1ccc2c(c1)c1cc(C(C)(C)C)ccc1n2-c1ccc2c(c1)Sc1cc(-n3c4ccccc4c4ccccc43)cc3c1B2c1ncn(-c2ccccc2)c1S3 ; c1ccc(-c2ccc3c(c2)Sc2cc(-n4c5ccccc5c5ccccc54)cc4c2B3c2ncn(-c3ccccc3)c2S4)cc1 ; c1ccc(-c2cc3c4c(c2)Sc2c(ncn2-c2ccccc2)B4c2ccc(-n4c5ccccc5c5ccccc54)cc2S3)cc1]

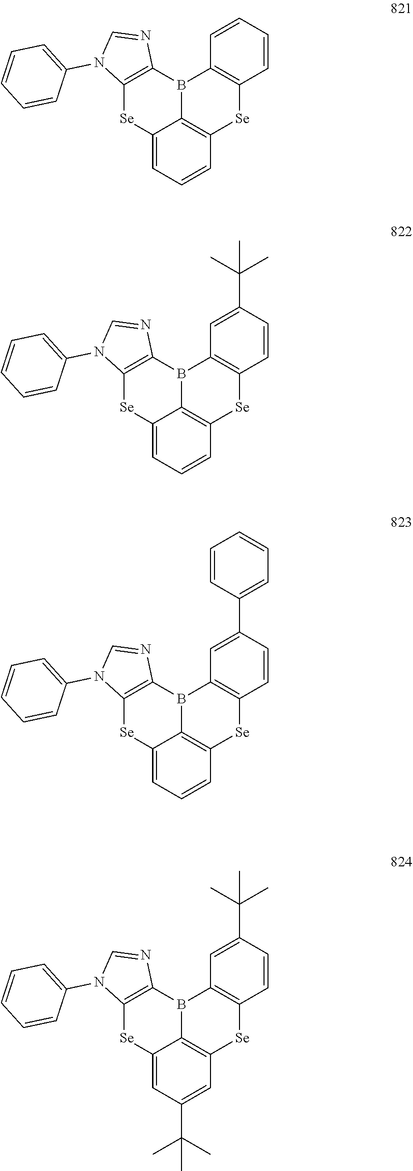 [SMILES: c1ccc(-n2cnc3c2[Se]c2cccc4c2B3c2ccccc2[Se]4)cc1 ; CC(C)(C)c1ccc2c(c1)B1c3ncn(-c4ccccc4)c3[Se]c3cccc(c31)[Se]2 ; c1ccc(-c2ccc3c(c2)B2c4ncn(-c5ccccc5)c4[Se]c4cccc(c42)[Se]3)cc1 ; CC(C)(C)c1cc2c3c(c1)[Se]c1c(ncn1-c1ccccc1)B3c1cc(C(C)(C)C)ccc1[Se]2]

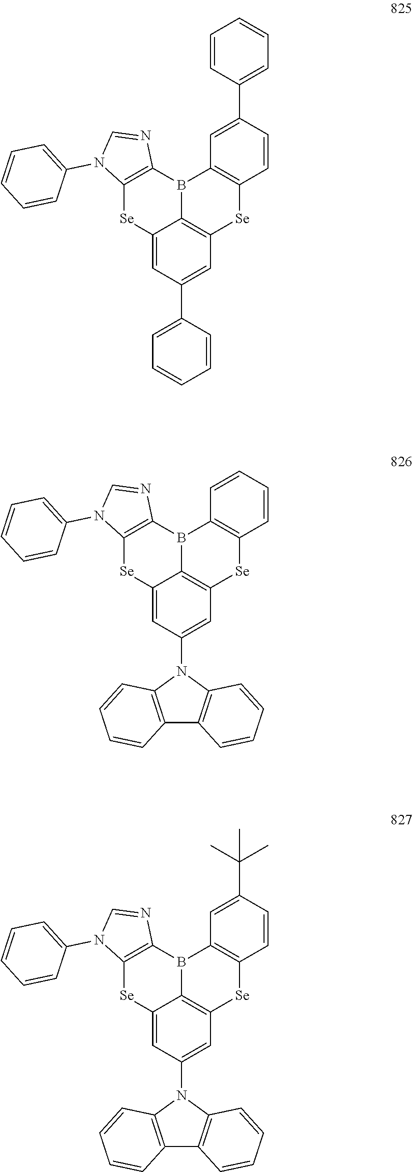 [SMILES: c1ccc(-c2cc3c4c(c2)[Se]c2c(ncn2-c2ccccc2)B4c2cc(-c4ccccc4)ccc2[Se]3)cc1 ; c1ccc(-n2cnc3c2[Se]c2cc(-n4c5ccccc5c5ccccc54)cc4c2B3c2ccccc2[Se]4)cc1 ; CC(C)(C)c1ccc2c(c1)B1c3ncn(-c4ccccc4)c3[Se]c3cc(-n4c5ccccc5c5ccccc54)cc(c31)[Se]2]

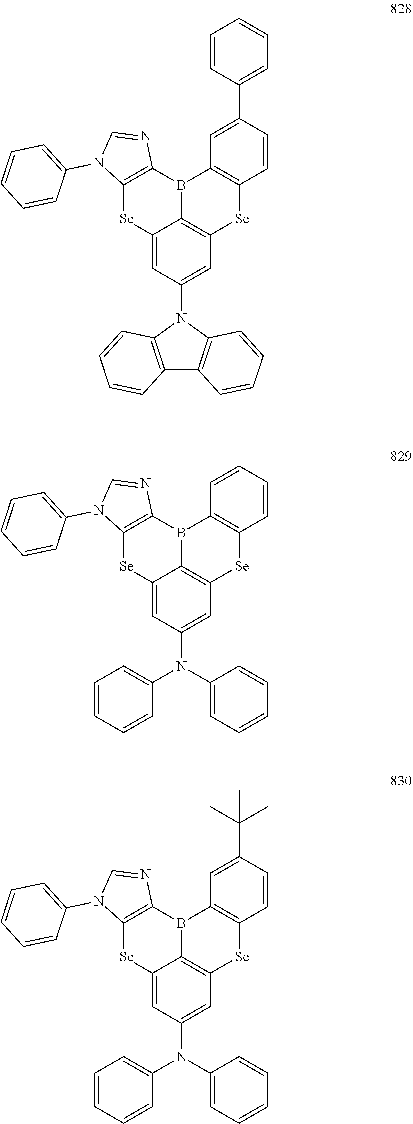 [SMILES: c1ccc(-c2ccc3c(c2)B2c4ncn(-c5ccccc5)c4[Se]c4cc(-n5c6ccccc6c6ccccc65)cc(c42)[Se]3)cc1 ; c1ccc(N(c2ccccc2)c2cc3c4c(c2)[Se]c2c(ncn2-c2ccccc2)B4c2ccccc2[Se]3)cc1 ; CC(C)(C)c1ccc2c(c1)B1c3ncn(-c4ccccc4)c3[Se]c3cc(N(c4ccccc4)c4ccccc4)cc(c31)[Se]2]

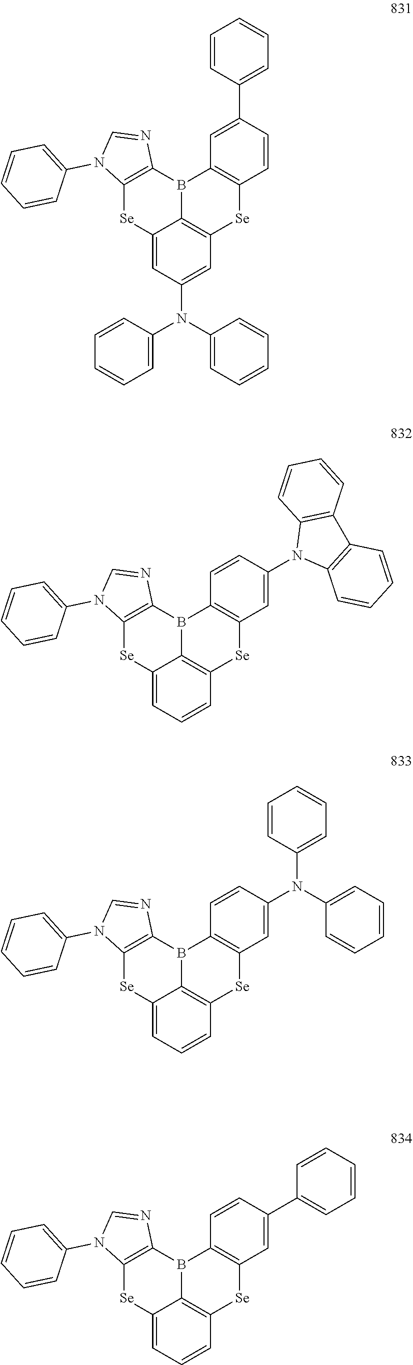 [SMILES: c1ccc(-c2ccc3c(c2)B2c4ncn(-c5ccccc5)c4[Se]c4cc(N(c5ccccc5)c5ccccc5)cc(c42)[Se]3)cc1 ; c1ccc(-n2cnc3c2[Se]c2cccc4c2B3c2ccc(-n3c5ccccc5c5ccccc53)cc2[Se]4)cc1 ; c1ccc(N(c2ccccc2)c2ccc3c(c2)[Se]c2cccc4c2B3c2ncn(-c3ccccc3)c2[Se]4)cc1 ; c1ccc(-c2ccc3c(c2)[Se]c2cccc4c2B3c2ncn(-c3ccccc3)c2[Se]4)cc1]

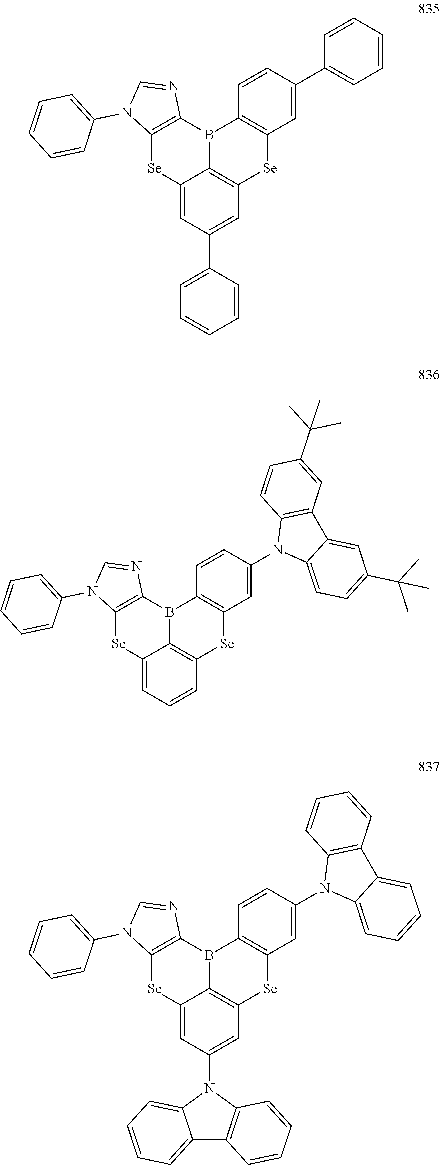 [SMILES: c1ccc(-c2ccc3c(c2)[Se]c2cc(-c4ccccc4)cc4c2B3c2ncn(-c3ccccc3)c2[Se]4)cc1 ; CC(C)(C)c1ccc2c(c1)c1cc(C(C)(C)C)ccc1n2-c1ccc2c(c1)[Se]c1cccc3c1B2c1ncn(-c2ccccc2)c1[Se]3 ; c1ccc(-n2cnc3c2[Se]c2cc(-n4c5ccccc5c5ccccc54)cc4c2B3c2ccc(-n3c5ccccc5c5ccccc53)cc2[Se]4)cc1]

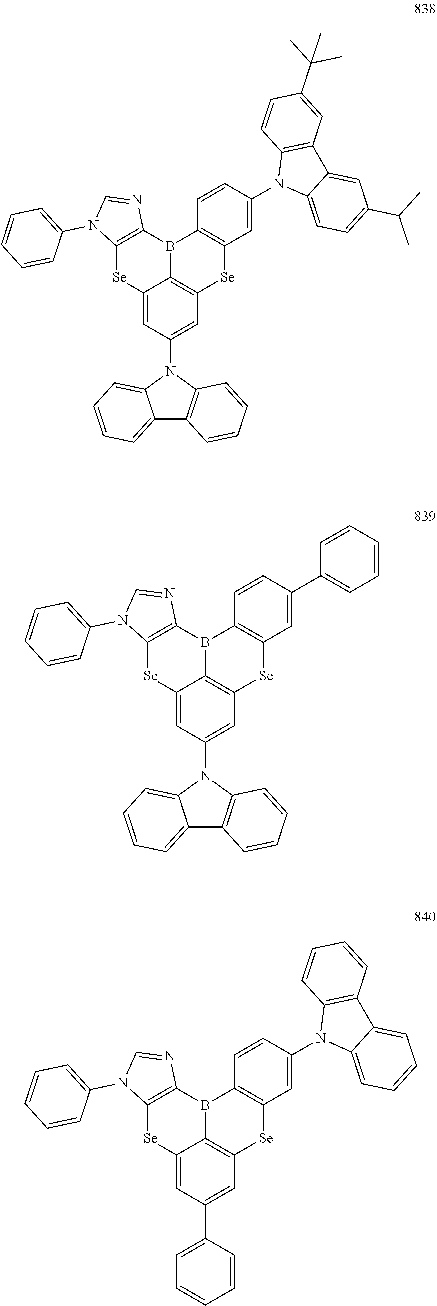 [SMILES: CC(C)c1ccc2c(c1)c1cc(C(C)(C)C)ccc1n2-c1ccc2c(c1)[Se]c1cc(-n3c4ccccc4c4ccccc43)cc3c1B2c1ncn(-c2ccccc2)c1[Se]3 ; c1ccc(-c2ccc3c(c2)[Se]c2cc(-n4c5ccccc5c5ccccc54)cc4c2B3c2ncn(-c3ccccc3)c2[Se]4)cc1 ; c1ccc(-c2cc3c4c(c2)[Se]c2c(ncn2-c2ccccc2)B4c2ccc(-n4c5ccccc5c5ccccc54)cc2[Se]3)cc1]

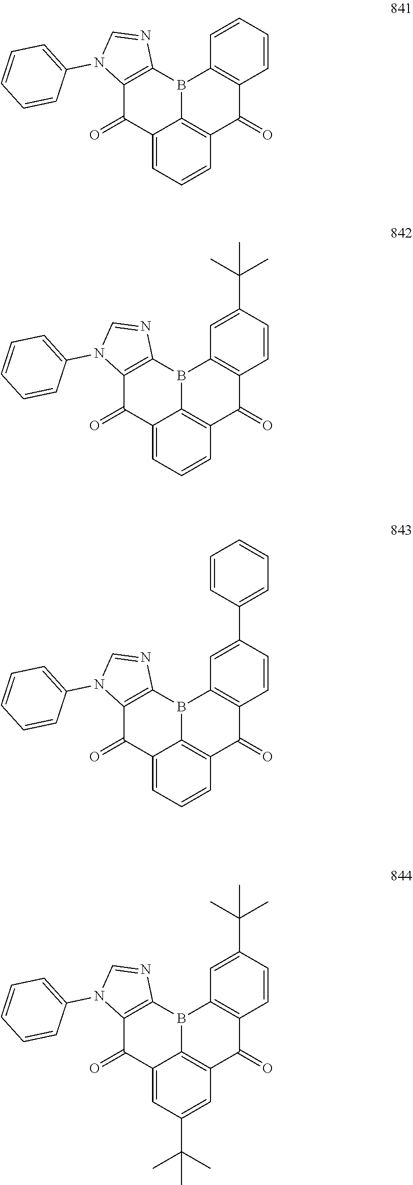 [SMILES: O=C1c2ccccc2B2c3ncn(-c4ccccc4)c3C(=O)c3cccc1c32 ; CC(C)(C)c1ccc2c(c1)B1c3ncn(-c4ccccc4)c3C(=O)c3cccc(c31)C2=O ; O=C1c2ccc(-c3ccccc3)cc2B2c3ncn(-c4ccccc4)c3C(=O)c3cccc1c32 ; CC(C)(C)c1ccc2c(c1)B1c3ncn(-c4ccccc4)c3C(=O)c3cc(C(C)(C)C)cc(c31)C2=O]

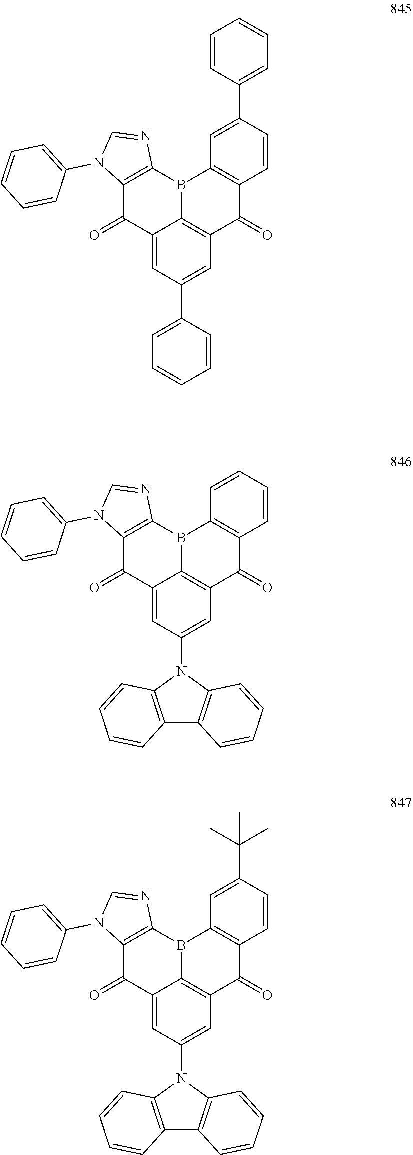 [SMILES: O=C1c2ccc(-c3ccccc3)cc2B2c3ncn(-c4ccccc4)c3C(=O)c3cc(-c4ccccc4)cc1c32 ; O=C1c2ccccc2B2c3ncn(-c4ccccc4)c3C(=O)c3cc(-n4c5ccccc5c5ccccc54)cc1c32 ; CC(C)(C)c1ccc2c(c1)B1c3ncn(-c4ccccc4)c3C(=O)c3cc(-n4c5ccccc5c5ccccc54)cc(c31)C2=O]

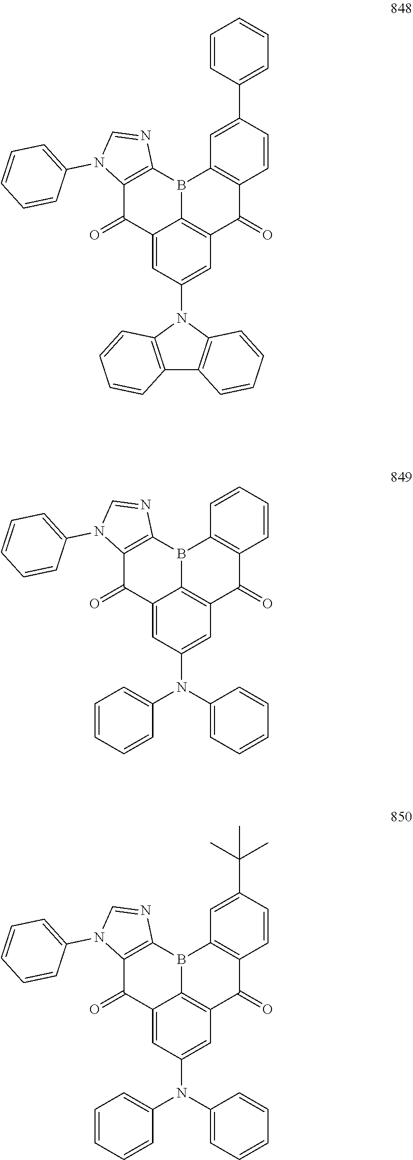 [SMILES: O=C1c2ccc(-c3ccccc3)cc2B2c3ncn(-c4ccccc4)c3C(=O)c3cc(-n4c5ccccc5c5ccccc54)cc1c32 ; O=C1c2ccccc2B2c3ncn(-c4ccccc4)c3C(=O)c3cc(N(c4ccccc4)c4ccccc4)cc1c32 ; CC(C)(C)c1ccc2c(c1)B1c3ncn(-c4ccccc4)c3C(=O)c3cc(N(c4ccccc4)c4ccccc4)cc(c31)C2=O]

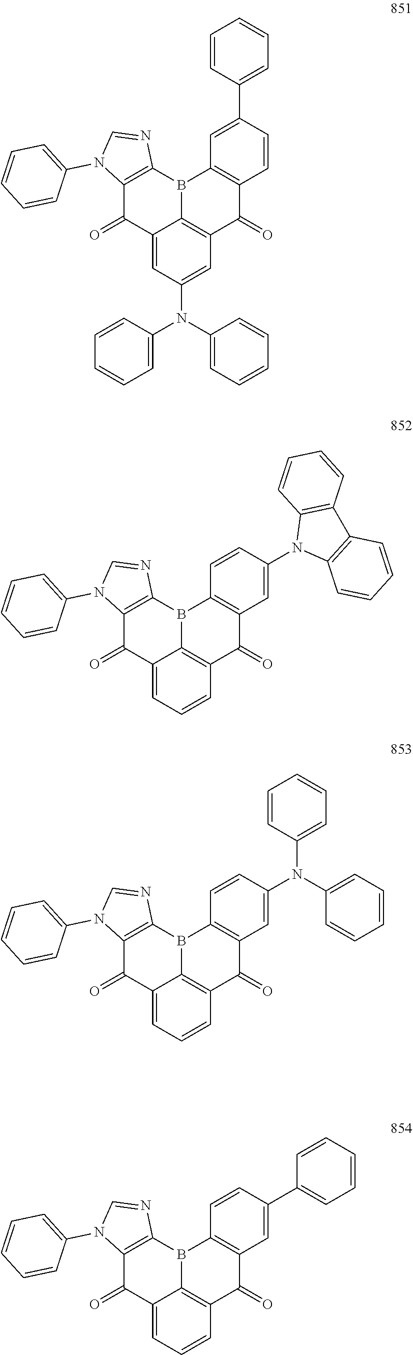 [SMILES: O=C1c2ccc(-c3ccccc3)cc2B2c3ncn(-c4ccccc4)c3C(=O)c3cc(N(c4ccccc4)c4ccccc4)cc1c32 ; O=C1c2cc(-n3c4ccccc4c4ccccc43)ccc2B2c3ncn(-c4ccccc4)c3C(=O)c3cccc1c32 ; O=C1c2cc(N(c3ccccc3)c3ccccc3)ccc2B2c3ncn(-c4ccccc4)c3C(=O)c3cccc1c32 ; O=C1c2cc(-c3ccccc3)ccc2B2c3ncn(-c4ccccc4)c3C(=O)c3cccc1c32]

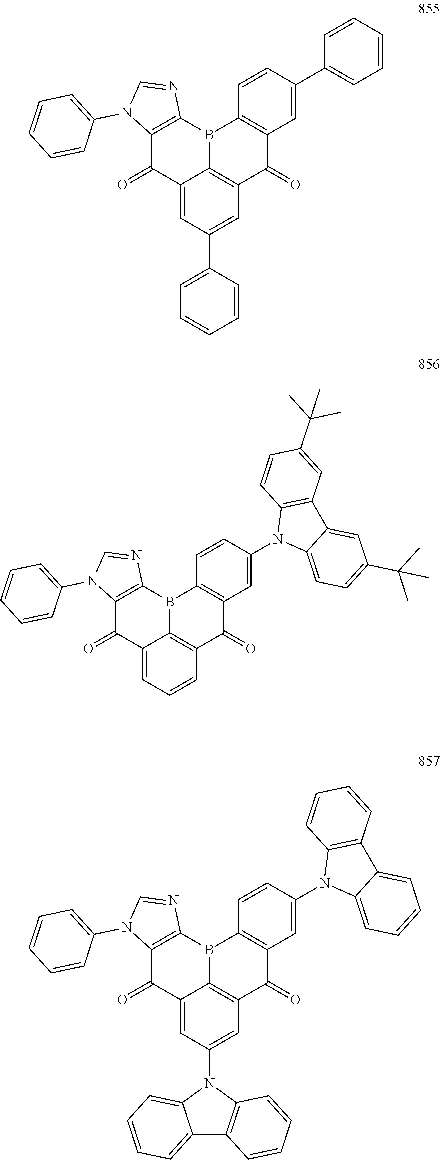 [SMILES: O=C1c2cc(-c3ccccc3)ccc2B2c3ncn(-c4ccccc4)c3C(=O)c3cc(-c4ccccc4)cc1c32 ; CC(C)(C)c1ccc2c(c1)c1cc(C(C)(C)C)ccc1n2-c1ccc2c(c1)C(=O)c1cccc3c1B2c1ncn(-c2ccccc2)c1C3=O ; O=C1c2cc(-n3c4ccccc4c4ccccc43)ccc2B2c3ncn(-c4ccccc4)c3C(=O)c3cc(-n4c5ccccc5c5ccccc54)cc1c32]

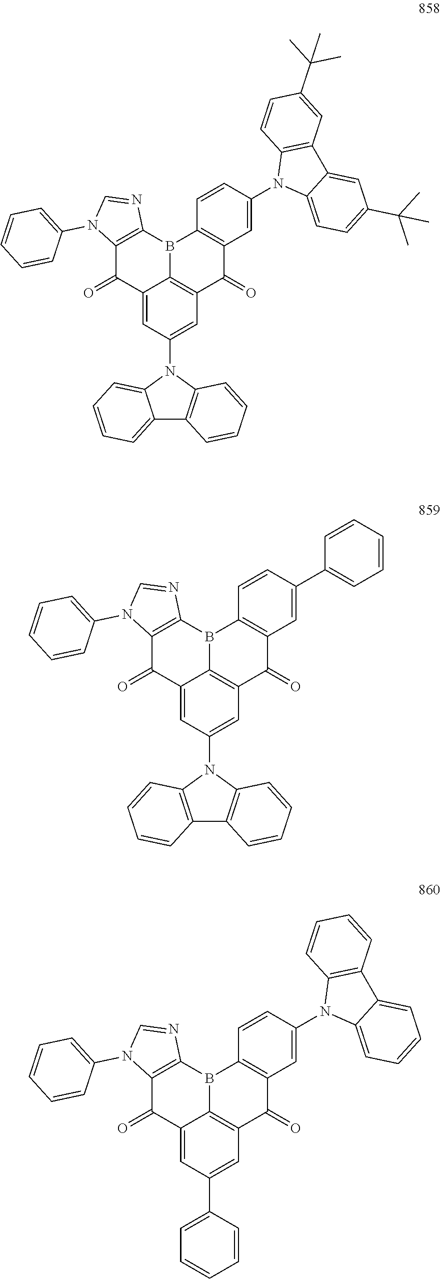 [SMILES: CC(C)(C)c1ccc2c(c1)c1cc(C(C)(C)C)ccc1n2-c1ccc2c(c1)C(=O)c1cc(-n3c4ccccc4c4ccccc43)cc3c1B2c1ncn(-c2ccccc2)c1C3=O ; O=C1c2cc(-c3ccccc3)ccc2B2c3ncn(-c4ccccc4)c3C(=O)c3cc(-n4c5ccccc5c5ccccc54)cc1c32 ; O=C1c2cc(-n3c4ccccc4c4ccccc43)ccc2B2c3ncn(-c4ccccc4)c3C(=O)c3cc(-c4ccccc4)cc1c32]

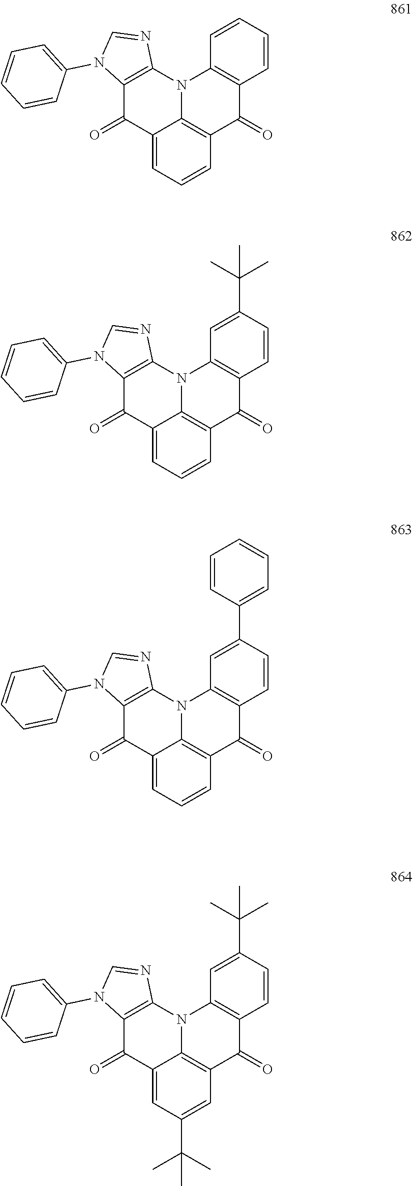 [SMILES: O=c1c2ccccc2n2c3ncn(-c4ccccc4)c3c(=O)c3cccc1c32 ; CC(C)(C)c1ccc2c(=O)c3cccc4c(=O)c5c(ncn5-c5ccccc5)n(c2c1)c34 ; O=c1c2ccc(-c3ccccc3)cc2n2c3ncn(-c4ccccc4)c3c(=O)c3cccc1c32 ; CC(C)(C)c1cc2c(=O)c3ccc(C(C)(C)C)cc3n3c4ncn(-c5ccccc5)c4c(=O)c(c1)c23]

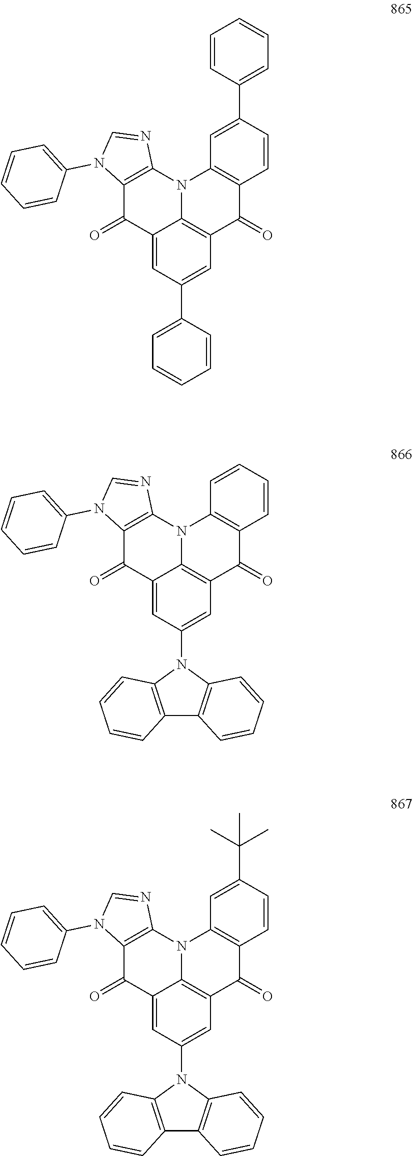 [SMILES: O=c1c2ccc(-c3ccccc3)cc2n2c3ncn(-c4ccccc4)c3c(=O)c3cc(-c4ccccc4)cc1c32 ; O=c1c2ccccc2n2c3ncn(-c4ccccc4)c3c(=O)c3cc(-n4c5ccccc5c5ccccc54)cc1c32 ; CC(C)(C)c1ccc2c(=O)c3cc(-n4c5ccccc5c5ccccc54)cc4c(=O)c5c(ncn5-c5ccccc5)n(c2c1)c34]

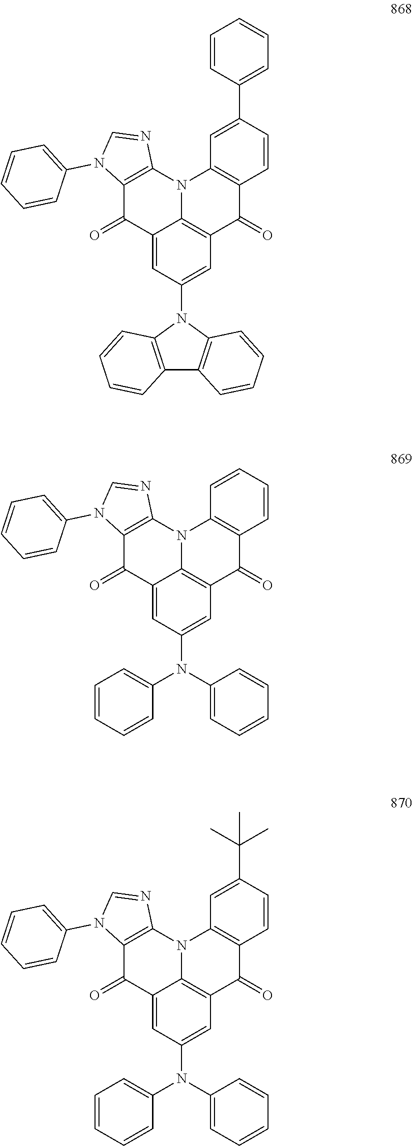 [SMILES: O=c1c2ccc(-c3ccccc3)cc2n2c3ncn(-c4ccccc4)c3c(=O)c3cc(-n4c5ccccc5c5ccccc54)cc1c32 ; O=c1c2ccccc2n2c3ncn(-c4ccccc4)c3c(=O)c3cc(N(c4ccccc4)c4ccccc4)cc1c32 ; CC(C)(C)c1ccc2c(=O)c3cc(N(c4ccccc4)c4ccccc4)cc4c(=O)c5c(ncn5-c5ccccc5)n(c2c1)c34]

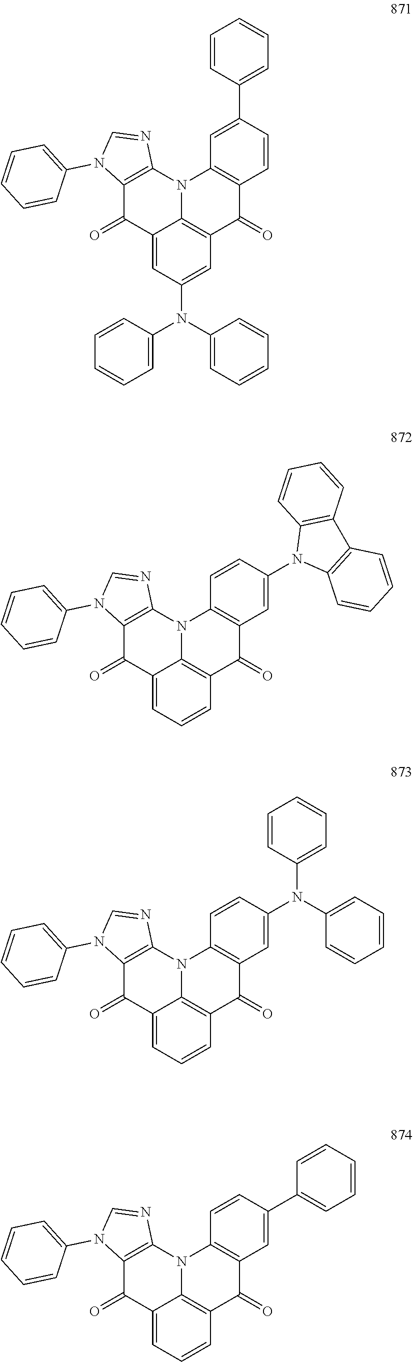 [SMILES: O=c1c2ccc(-c3ccccc3)cc2n2c3ncn(-c4ccccc4)c3c(=O)c3cc(N(c4ccccc4)c4ccccc4)cc1c32 ; O=c1c2cc(-n3c4ccccc4c4ccccc43)ccc2n2c3ncn(-c4ccccc4)c3c(=O)c3cccc1c32 ; O=c1c2cc(N(c3ccccc3)c3ccccc3)ccc2n2c3ncn(-c4ccccc4)c3c(=O)c3cccc1c32 ; O=c1c2cc(-c3ccccc3)ccc2n2c3ncn(-c4ccccc4)c3c(=O)c3cccc1c32]

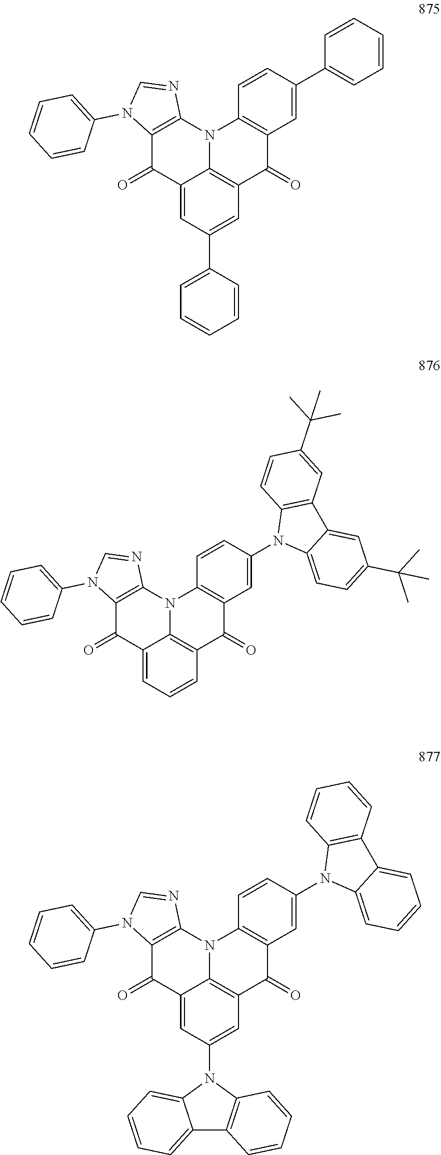 [SMILES: O=c1c2cc(-c3ccccc3)ccc2n2c3ncn(-c4ccccc4)c3c(=O)c3cc(-c4ccccc4)cc1c32 ; CC(C)(C)c1ccc2c(c1)c1cc(C(C)(C)C)ccc1n2-c1ccc2c(c1)c(=O)c1cccc3c(=O)c4c(ncn4-c4ccccc4)n2c13 ; O=c1c2cc(-n3c4ccccc4c4ccccc43)ccc2n2c3ncn(-c4ccccc4)c3c(=O)c3cc(-n4c5ccccc5c5ccccc54)cc1c32]

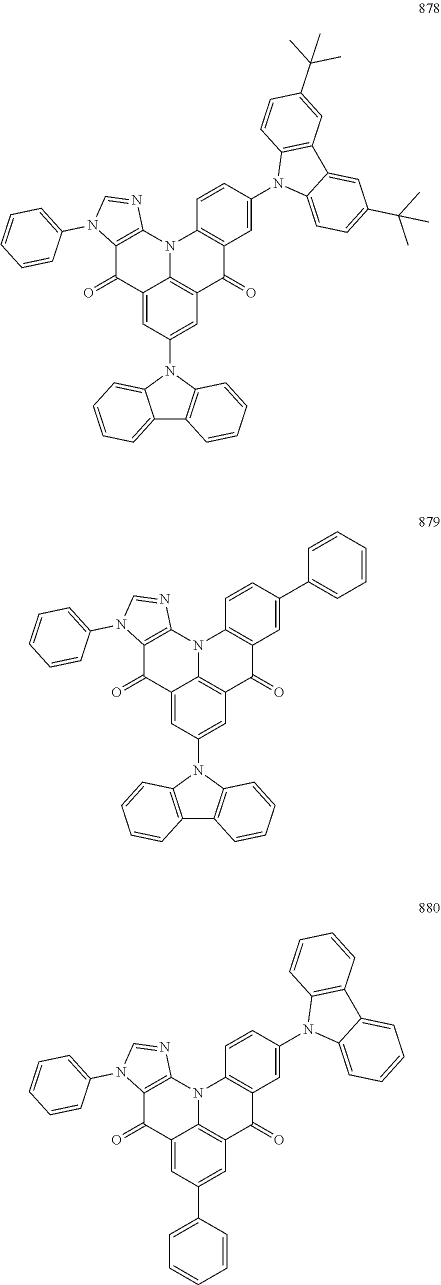 [SMILES: CC(C)(C)c1ccc2c(c1)c1cc(C(C)(C)C)ccc1n2-c1ccc2c(c1)c(=O)c1cc(-n3c4ccccc4c4ccccc43)cc3c(=O)c4c(ncn4-c4ccccc4)n2c13 ; O=c1c2cc(-c3ccccc3)ccc2n2c3ncn(-c4ccccc4)c3c(=O)c3cc(-n4c5ccccc5c5ccccc54)cc1c32 ; O=c1c2cc(-n3c4ccccc4c4ccccc43)ccc2n2c3ncn(-c4ccccc4)c3c(=O)c3cc(-c4ccccc4)cc1c32]

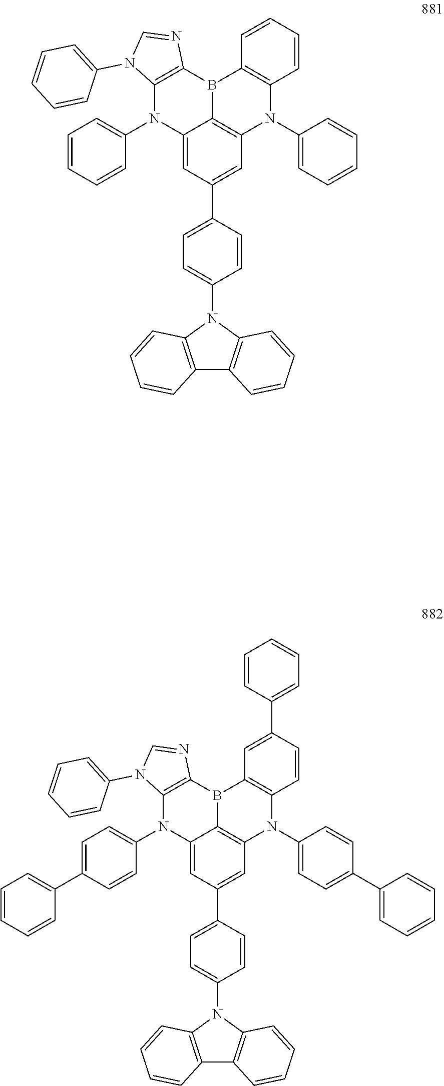 [SMILES: c1ccc(N2c3ccccc3B3c4ncn(-c5ccccc5)c4N(c4ccccc4)c4cc(-c5ccc(-n6c7ccccc7c7ccccc76)cc5)cc2c43)cc1 ; c1ccc(-c2ccc(N3c4ccc(-c5ccccc5)cc4B4c5ncn(-c6ccccc6)c5N(c5ccc(-c6ccccc6)cc5)c5cc(-c6ccc(-n7c8ccccc8c8ccccc87)cc6)cc3c54)cc2)cc1]

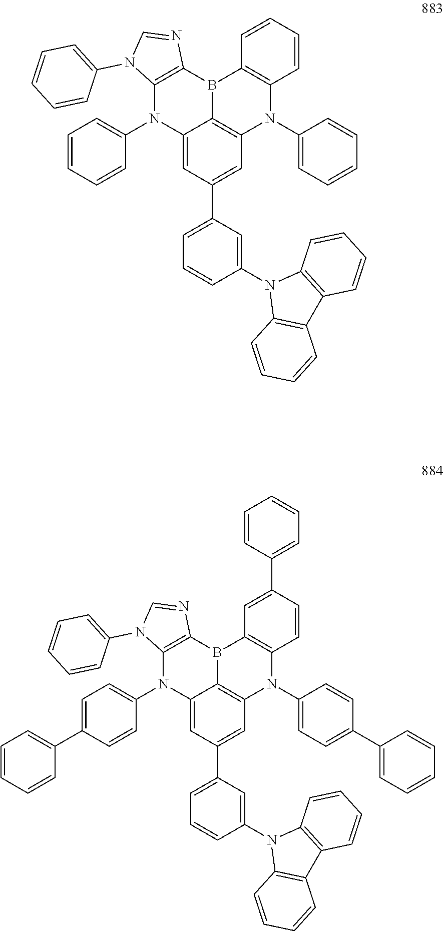 [SMILES: c1ccc(N2c3ccccc3B3c4ncn(-c5ccccc5)c4N(c4ccccc4)c4cc(-c5cccc(-n6c7ccccc7c7ccccc76)c5)cc2c43)cc1 ; c1ccc(-c2ccc(N3c4ccc(-c5ccccc5)cc4B4c5ncn(-c6ccccc6)c5N(c5ccc(-c6ccccc6)cc5)c5cc(-c6cccc(-n7c8ccccc8c8ccccc87)c6)cc3c54)cc2)cc1]

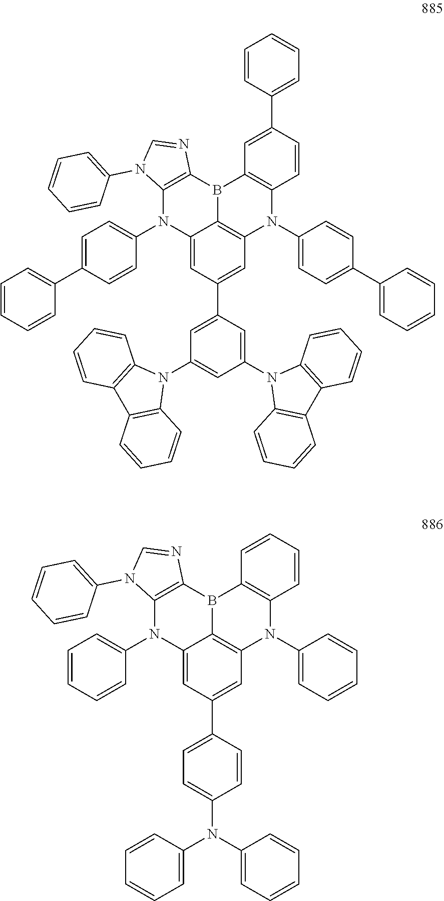 [SMILES: c1ccc(-c2ccc(N3c4ccc(-c5ccccc5)cc4B4c5ncn(-c6ccccc6)c5N(c5ccc(-c6ccccc6)cc5)c5cc(-c6cc(-n7c8ccccc8c8ccccc87)cc(-n7c8ccccc8c8ccccc87)c6)cc3c54)cc2)cc1 ; c1ccc(N(c2ccccc2)c2ccc(-c3cc4c5c(c3)N(c3ccccc3)c3c(ncn3-c3ccccc3)B5c3ccccc3N4c3ccccc3)cc2)cc1]

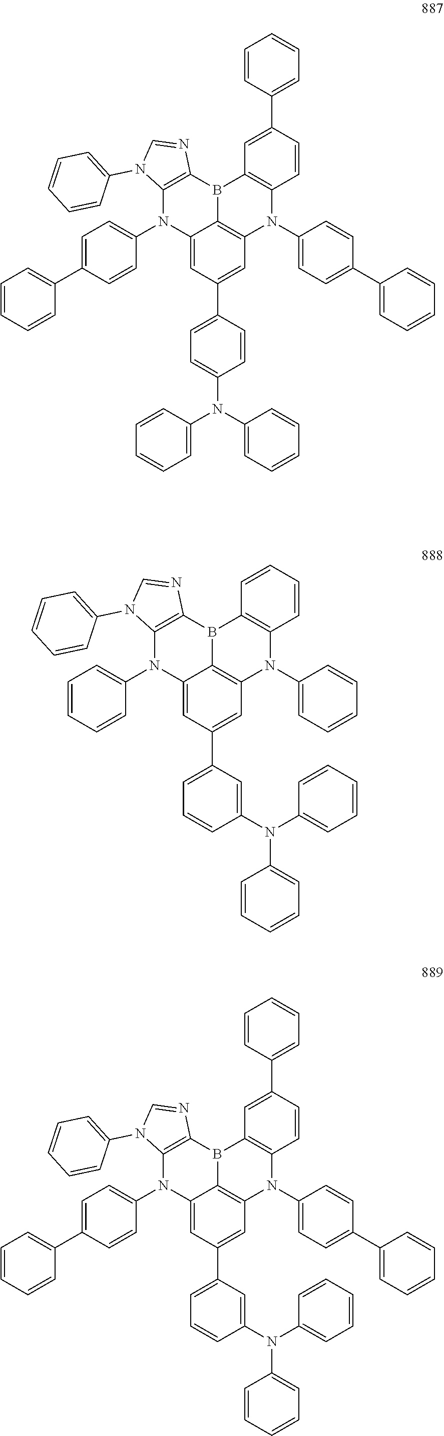 [SMILES: c1ccc(-c2ccc(N3c4ccc(-c5ccccc5)cc4B4c5ncn(-c6ccccc6)c5N(c5ccc(-c6ccccc6)cc5)c5cc(-c6ccc(N(c7ccccc7)c7ccccc7)cc6)cc3c54)cc2)cc1 ; c1ccc(N(c2ccccc2)c2cccc(-c3cc4c5c(c3)N(c3ccccc3)c3c(ncn3-c3ccccc3)B5c3ccccc3N4c3ccccc3)c2)cc1 ; c1ccc(-c2ccc(N3c4ccc(-c5ccccc5)cc4B4c5ncn(-c6ccccc6)c5N(c5ccc(-c6ccccc6)cc5)c5cc(-c6cccc(N(c7ccccc7)c7ccccc7)c6)cc3c54)cc2)cc1]

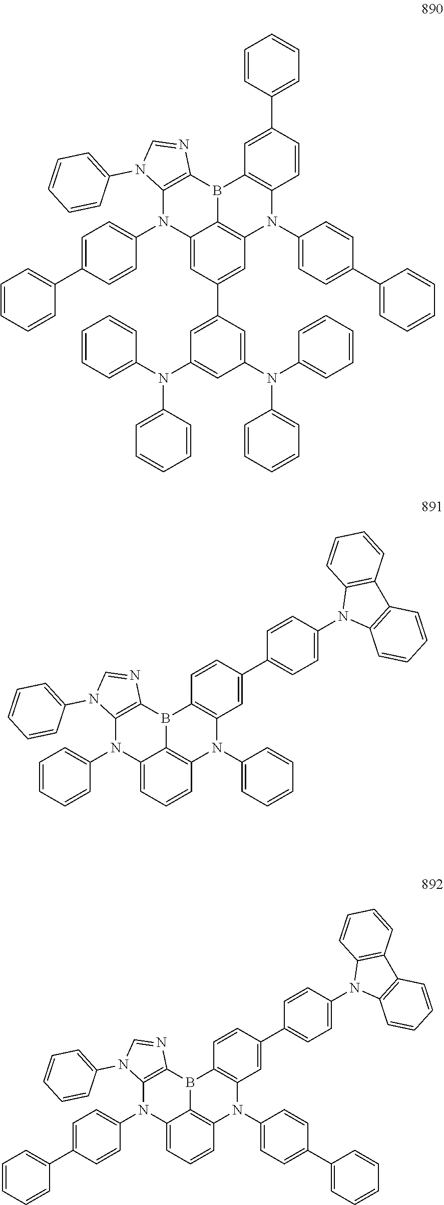 [SMILES: c1ccc(-c2ccc(N3c4ccc(-c5ccccc5)cc4B4c5ncn(-c6ccccc6)c5N(c5ccc(-c6ccccc6)cc5)c5cc(-c6cc(N(c7ccccc7)c7ccccc7)cc(N(c7ccccc7)c7ccccc7)c6)cc3c54)cc2)cc1 ; c1ccc(N2c3cc(-c4ccc(-n5c6ccccc6c6ccccc65)cc4)ccc3B3c4ncn(-c5ccccc5)c4N(c4ccccc4)c4cccc2c43)cc1 ; c1ccc(-c2ccc(N3c4cc(-c5ccc(-n6c7ccccc7c7ccccc76)cc5)ccc4B4c5ncn(-c6ccccc6)c5N(c5ccc(-c6ccccc6)cc5)c5cccc3c54)cc2)cc1]

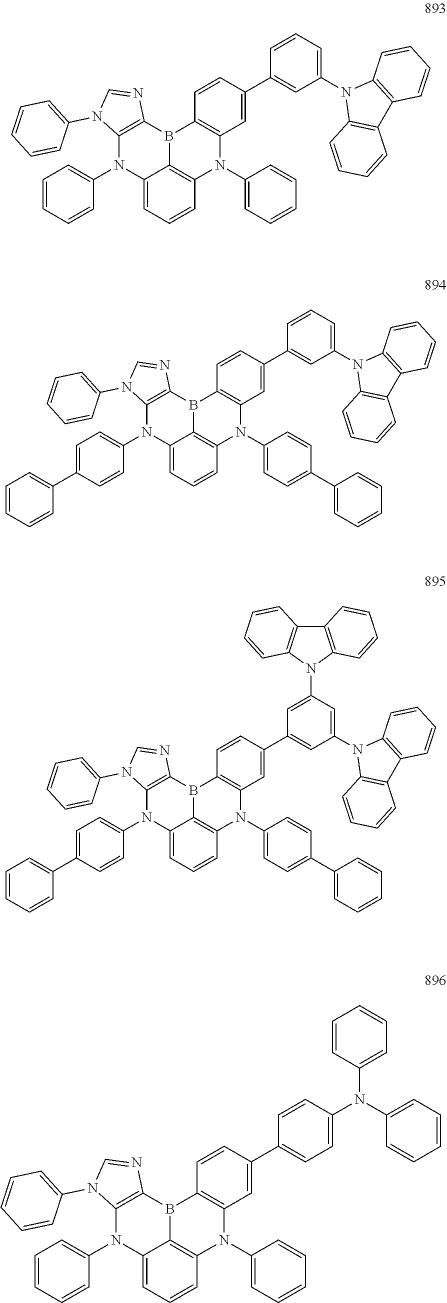 [SMILES: c1ccc(N2c3cc(-c4cccc(-n5c6ccccc6c6ccccc65)c4)ccc3B3c4ncn(-c5ccccc5)c4N(c4ccccc4)c4cccc2c43)cc1 ; c1ccc(-c2ccc(N3c4cc(-c5cccc(-n6c7ccccc7c7ccccc76)c5)ccc4B4c5ncn(-c6ccccc6)c5N(c5ccc(-c6ccccc6)cc5)c5cccc3c54)cc2)cc1 ; c1ccc(-c2ccc(N3c4cc(-c5cc(-n6c7ccccc7c7ccccc76)cc(-n6c7ccccc7c7ccccc76)c5)ccc4B4c5ncn(-c6ccccc6)c5N(c5ccc(-c6ccccc6)cc5)c5cccc3c54)cc2)cc1 ; c1ccc(N(c2ccccc2)c2ccc(-c3ccc4c(c3)N(c3ccccc3)c3cccc5c3B4c3ncn(-c4ccccc4)c3N5c3ccccc3)cc2)cc1]

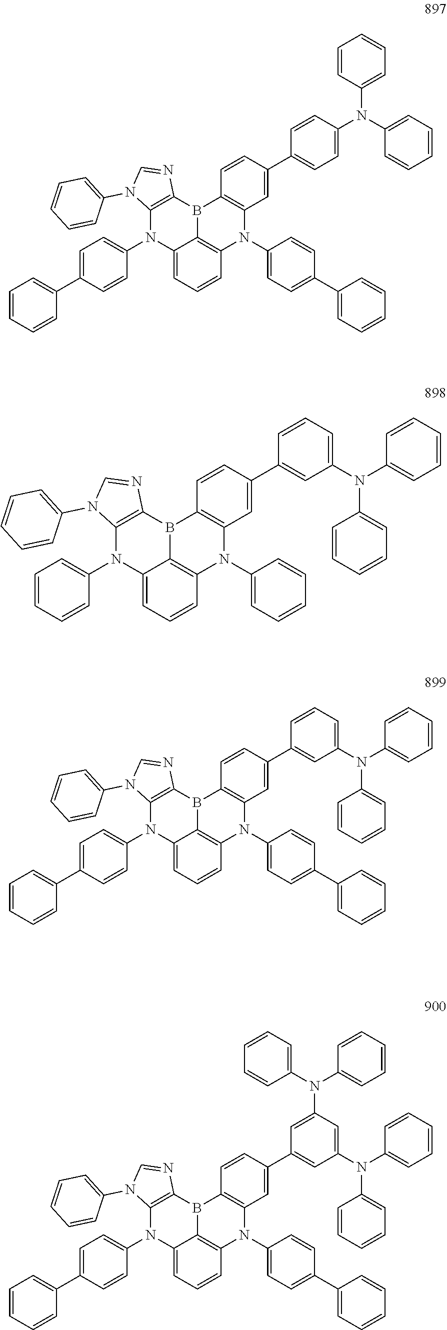 [SMILES: c1ccc(-c2ccc(N3c4cc(-c5ccc(N(c6ccccc6)c6ccccc6)cc5)ccc4B4c5ncn(-c6ccccc6)c5N(c5ccc(-c6ccccc6)cc5)c5cccc3c54)cc2)cc1 ; c1ccc(N(c2ccccc2)c2cccc(-c3ccc4c(c3)N(c3ccccc3)c3cccc5c3B4c3ncn(-c4ccccc4)c3N5c3ccccc3)c2)cc1 ; c1ccc(-c2ccc(N3c4cc(-c5cccc(N(c6ccccc6)c6ccccc6)c5)ccc4B4c5ncn(-c6ccccc6)c5N(c5ccc(-c6ccccc6)cc5)c5cccc3c54)cc2)cc1 ; c1ccc(-c2ccc(N3c4cc(-c5cc(N(c6ccccc6)c6ccccc6)cc(N(c6ccccc6)c6ccccc6)c5)ccc4B4c5ncn(-c6ccccc6)c5N(c5ccc(-c6ccccc6)cc5)c5cccc3c54)cc2)cc1]

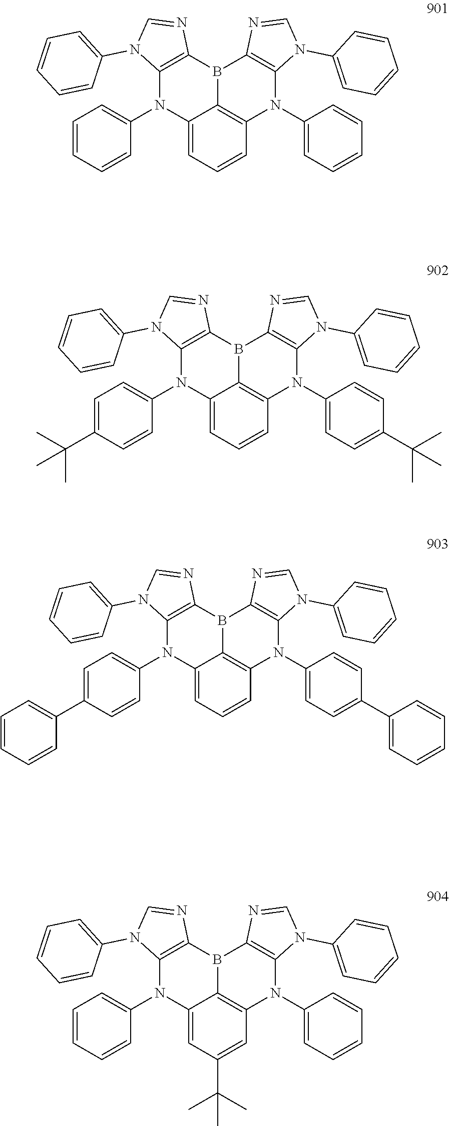 [SMILES: c1ccc(N2c3cccc4c3B(c3ncn(-c5ccccc5)c32)c2ncn(-c3ccccc3)c2N4c2ccccc2)cc1 ; CC(C)(C)c1ccc(N2c3cccc4c3B(c3ncn(-c5ccccc5)c32)c2ncn(-c3ccccc3)c2N4c2ccc(C(C)(C)C)cc2)cc1 ; c1ccc(-c2ccc(N3c4cccc5c4B(c4ncn(-c6ccccc6)c43)c3ncn(-c4ccccc4)c3N5c3ccc(-c4ccccc4)cc3)cc2)cc1 ; CC(C)(C)c1cc2c3c(c1)N(c1ccccc1)c1c(ncn1-c1ccccc1)B3c1ncn(-c3ccccc3)c1N2c1ccccc1]

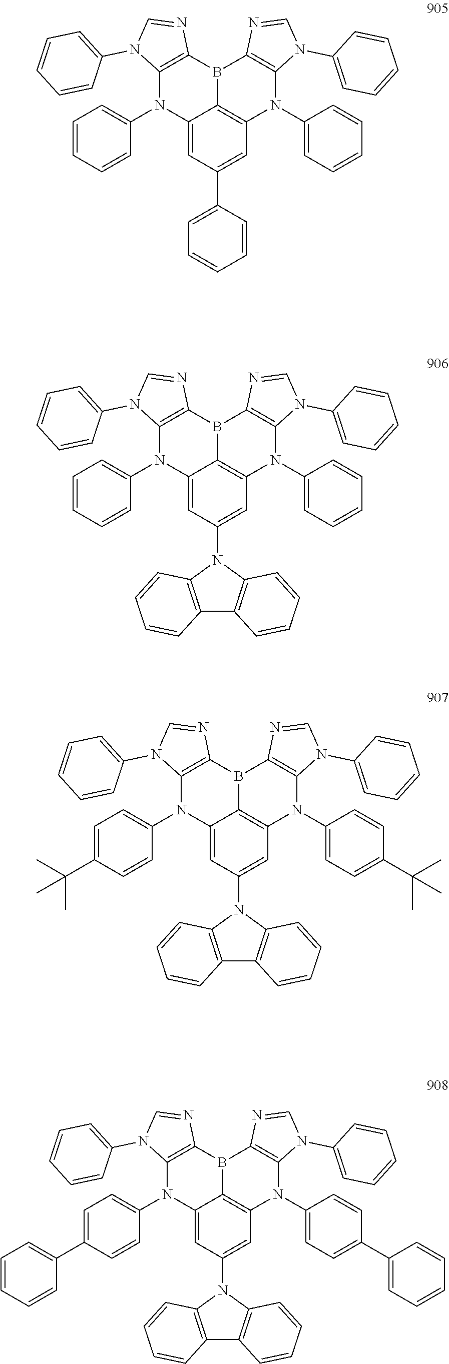 [SMILES: c1ccc(-c2cc3c4c(c2)N(c2ccccc2)c2c(ncn2-c2ccccc2)B4c2ncn(-c4ccccc4)c2N3c2ccccc2)cc1 ; c1ccc(N2c3cc(-n4c5ccccc5c5ccccc54)cc4c3B(c3ncn(-c5ccccc5)c32)c2ncn(-c3ccccc3)c2N4c2ccccc2)cc1 ; CC(C)(C)c1ccc(N2c3cc(-n4c5ccccc5c5ccccc54)cc4c3B(c3ncn(-c5ccccc5)c32)c2ncn(-c3ccccc3)c2N4c2ccc(C(C)(C)C)cc2)cc1 ; c1ccc(-c2ccc(N3c4cc(-n5c6ccccc6c6ccccc65)cc5c4B(c4ncn(-c6ccccc6)c43)c3ncn(-c4ccccc4)c3N5c3ccc(-c4ccccc4)cc3)cc2)cc1]

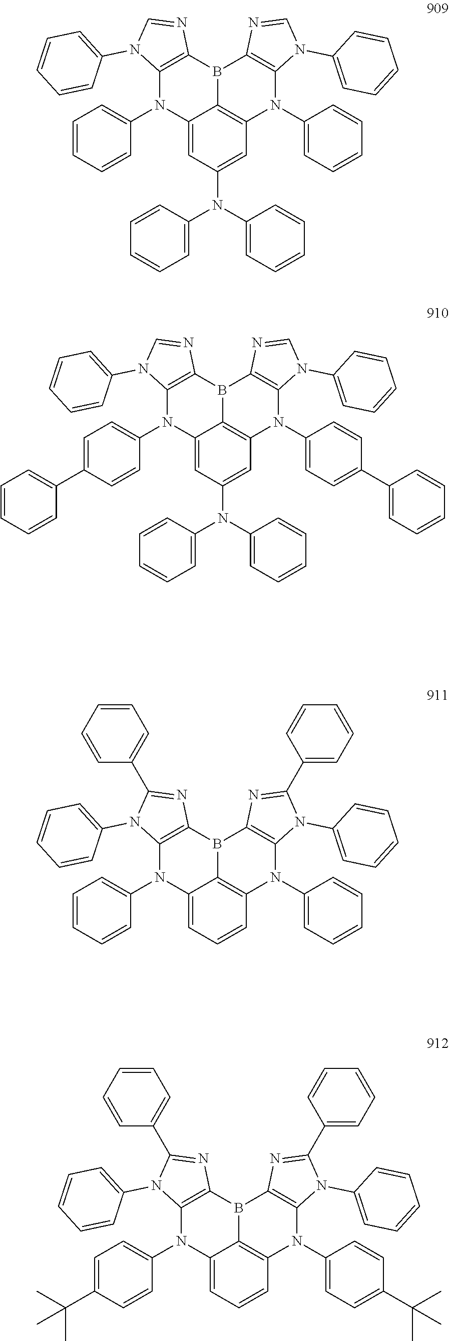 [SMILES: c1ccc(N(c2ccccc2)c2cc3c4c(c2)N(c2ccccc2)c2c(ncn2-c2ccccc2)B4c2ncn(-c4ccccc4)c2N3c2ccccc2)cc1 ; c1ccc(-c2ccc(N3c4cc(N(c5ccccc5)c5ccccc5)cc5c4B(c4ncn(-c6ccccc6)c43)c3ncn(-c4ccccc4)c3N5c3ccc(-c4ccccc4)cc3)cc2)cc1 ; c1ccc(-c2nc3c(n2-c2ccccc2)N(c2ccccc2)c2cccc4c2B3c2nc(-c3ccccc3)n(-c3ccccc3)c2N4c2ccccc2)cc1 ; CC(C)(C)c1ccc(N2c3cccc4c3B(c3nc(-c5ccccc5)n(-c5ccccc5)c32)c2nc(-c3ccccc3)n(-c3ccccc3)c2N4c2ccc(C(C)(C)C)cc2)cc1]

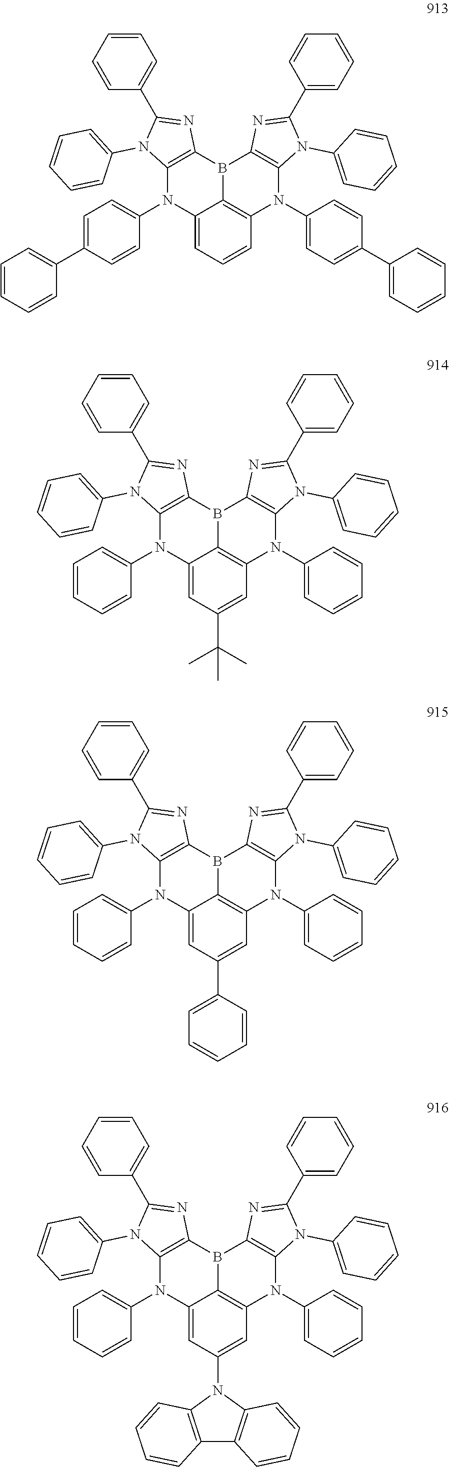 [SMILES: c1ccc(-c2ccc(N3c4cccc5c4B(c4nc(-c6ccccc6)n(-c6ccccc6)c43)c3nc(-c4ccccc4)n(-c4ccccc4)c3N5c3ccc(-c4ccccc4)cc3)cc2)cc1 ; CC(C)(C)c1cc2c3c(c1)N(c1ccccc1)c1c(nc(-c4ccccc4)n1-c1ccccc1)B3c1nc(-c3ccccc3)n(-c3ccccc3)c1N2c1ccccc1 ; c1ccc(-c2cc3c4c(c2)N(c2ccccc2)c2c(nc(-c5ccccc5)n2-c2ccccc2)B4c2nc(-c4ccccc4)n(-c4ccccc4)c2N3c2ccccc2)cc1 ; c1ccc(-c2nc3c(n2-c2ccccc2)N(c2ccccc2)c2cc(-n4c5ccccc5c5ccccc54)cc4c2B3c2nc(-c3ccccc3)n(-c3ccccc3)c2N4c2ccccc2)cc1]

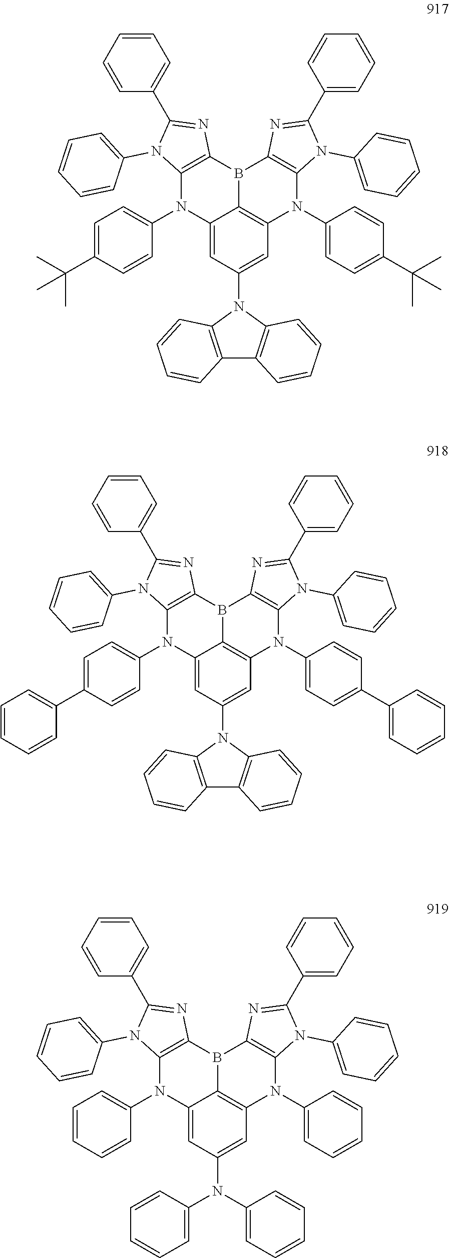 [SMILES: CC(C)(C)c1ccc(N2c3cc(-n4c5ccccc5c5ccccc54)cc4c3B(c3nc(-c5ccccc5)n(-c5ccccc5)c32)c2nc(-c3ccccc3)n(-c3ccccc3)c2N4c2ccc(C(C)(C)C)cc2)cc1 ; c1ccc(-c2ccc(N3c4cc(-n5c6ccccc6c6ccccc65)cc5c4B(c4nc(-c6ccccc6)n(-c6ccccc6)c43)c3nc(-c4ccccc4)n(-c4ccccc4)c3N5c3ccc(-c4ccccc4)cc3)cc2)cc1 ; c1ccc(-c2nc3c(n2-c2ccccc2)N(c2ccccc2)c2cc(N(c4ccccc4)c4ccccc4)cc4c2B3c2nc(-c3ccccc3)n(-c3ccccc3)c2N4c2ccccc2)cc1]

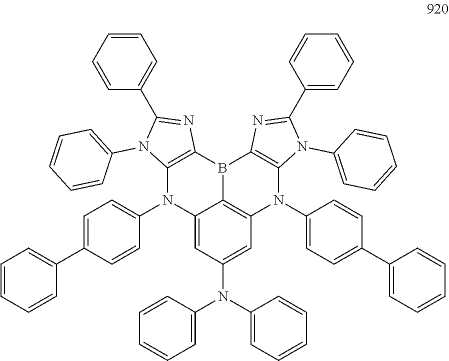 [SMILES: c1ccc(-c2ccc(N3c4cc(N(c5ccccc5)c5ccccc5)cc5c4B(c4nc(-c6ccccc6)n(-c6ccccc6)c43)c3nc(-c4ccccc4)n(-c4ccccc4)c3N5c3ccc(-c4ccccc4)cc3)cc2)cc1]

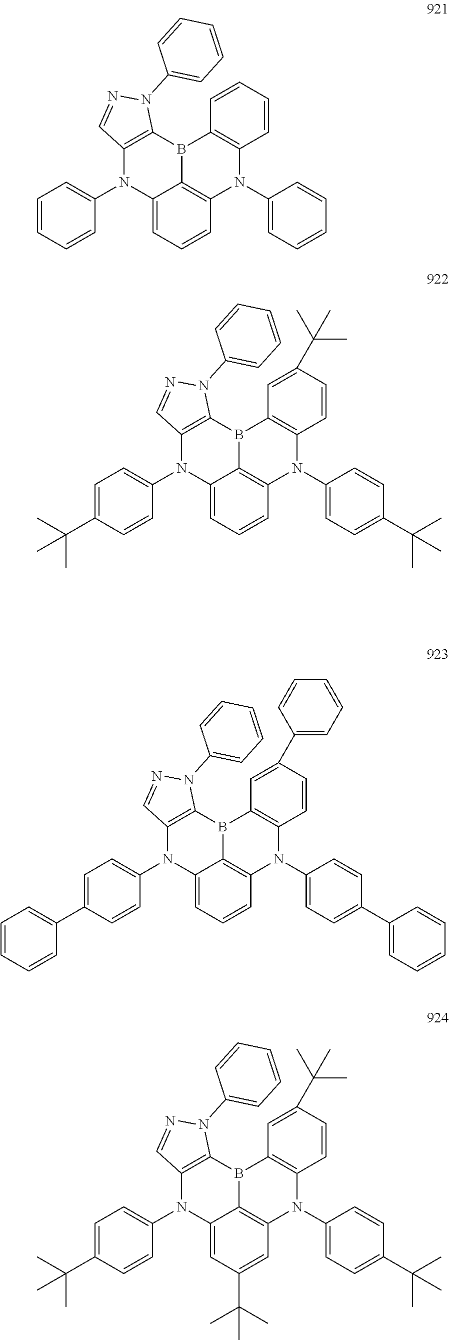

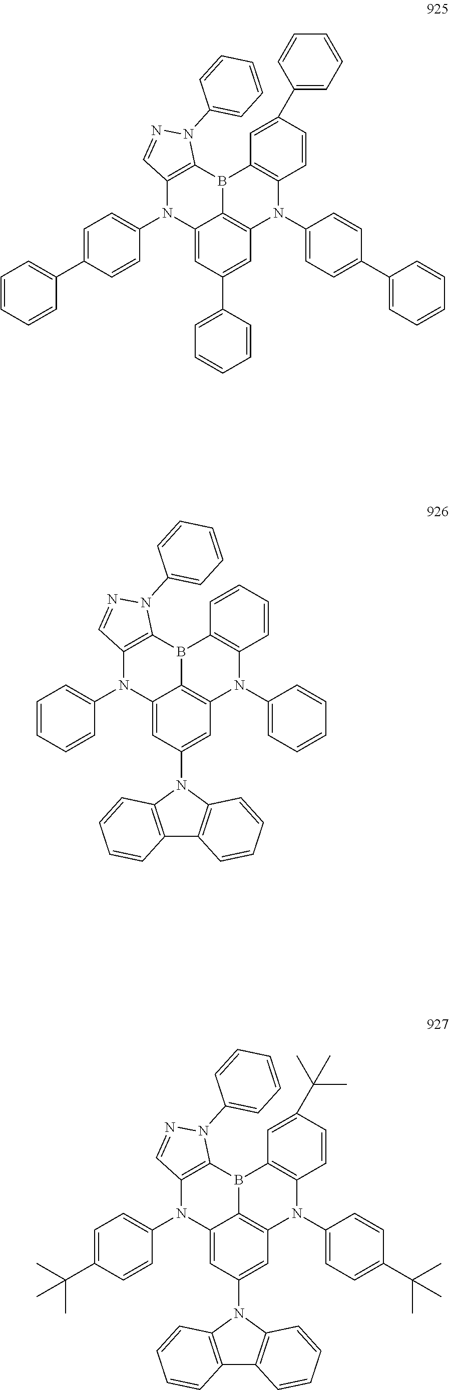 [SMILES: c1ccc(-c2ccc(N3c4ccc(-c5ccccc5)cc4B4c5c3cc(-c3ccccc3)cc5N(c3ccc(-c5ccccc5)cc3)c3cnn(-c5ccccc5)c34)cc2)cc1 ; c1ccc(N2c3ccccc3B3c4c2cc(-n2c5ccccc5c5ccccc52)cc4N(c2ccccc2)c2cnn(-c4ccccc4)c23)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2cnn(-c4ccccc4)c23)cc1]

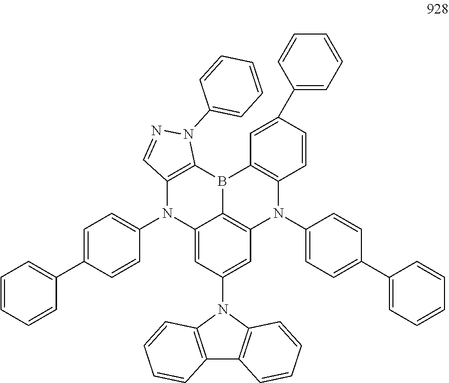 [SMILES: c1ccc(-c2ccc(N3c4ccc(-c5ccccc5)cc4B4c5c3cc(-n3c6ccccc6c6ccccc63)cc5N(c3ccc(-c5ccccc5)cc3)c3cnn(-c5ccccc5)c34)cc2)cc1]

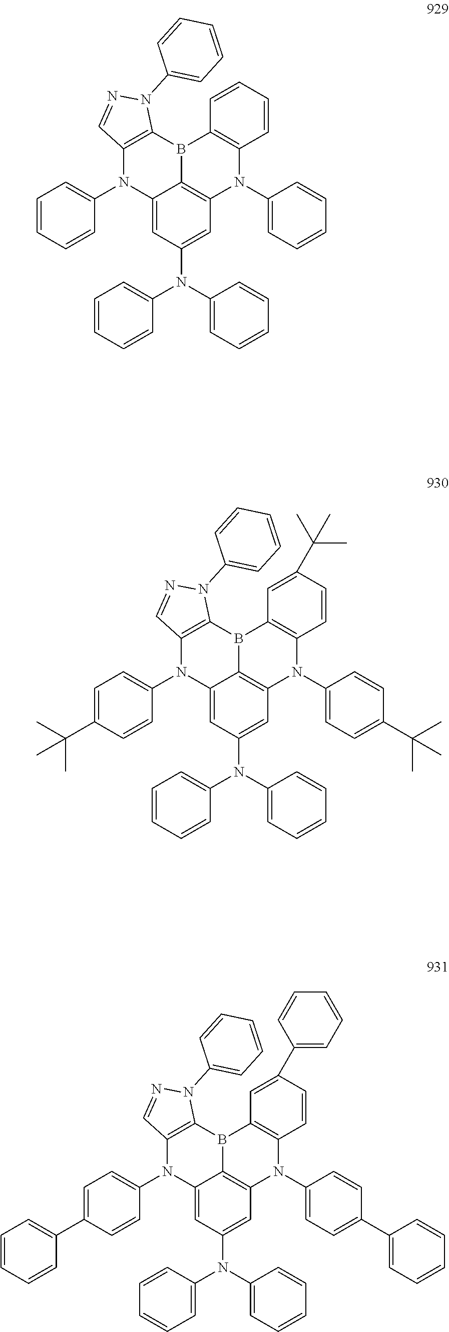 [SMILES: c1ccc(N(c2ccccc2)c2cc3c4c(c2)N(c2ccccc2)c2cnn(-c5ccccc5)c2B4c2ccccc2N3c2ccccc2)cc1 ; CC(C)(C)c1ccc(N2c3ccc(C(C)(C)C)cc3B3c4c2cc(N(c2ccccc2)c2ccccc2)cc4N(c2ccc(C(C)(C)C)cc2)c2cnn(-c4ccccc4)c23)cc1 ; c1ccc(-c2ccc(N3c4ccc(-c5ccccc5)cc4B4c5c3cc(N(c3ccccc3)c3ccccc3)cc5N(c3ccc(-c5ccccc5)cc3)c3cnn(-c5ccccc5)c34)cc2)cc1]

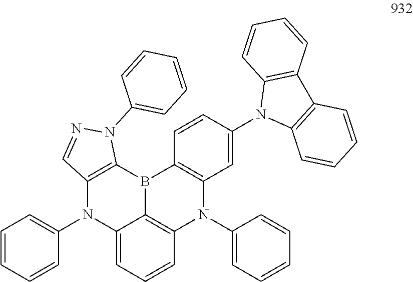 [SMILES: c1ccc(N2c3cc(-n4c5ccccc5c5ccccc54)ccc3B3c4c2cccc4N(c2ccccc2)c2cnn(-c4ccccc4)c23)cc1]

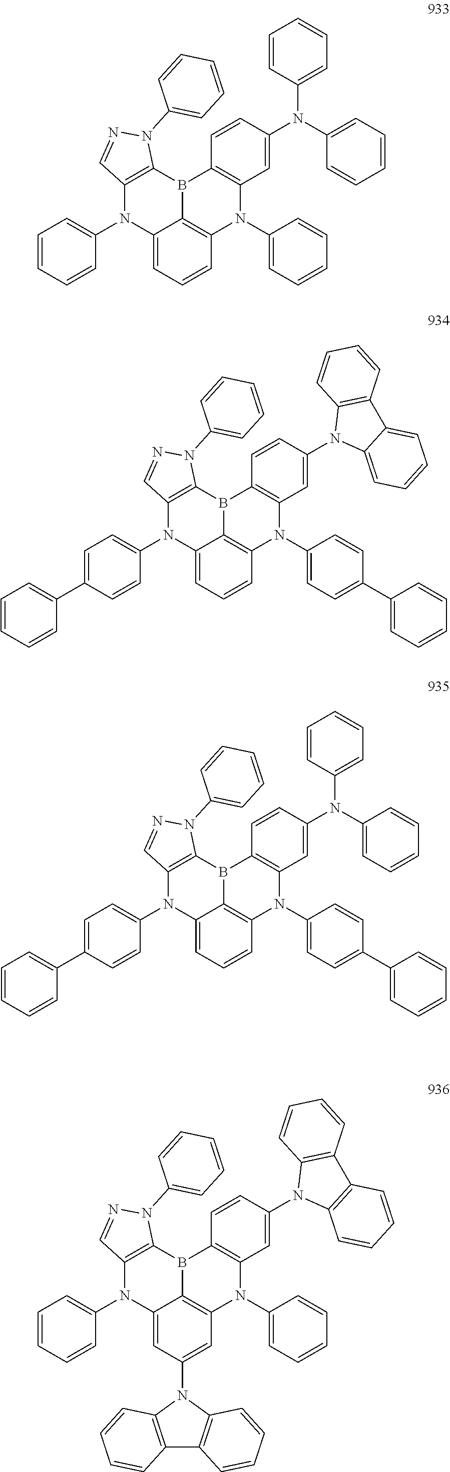 [SMILES: c1ccc(N(c2ccccc2)c2ccc3c(c2)N(c2ccccc2)c2cccc4c2B3c2c(cnn2-c2ccccc2)N4c2ccccc2)cc1 ; c1ccc(-c2ccc(N3c4cc(-n5c6ccccc6c6ccccc65)ccc4B4c5c3cccc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3cnn(-c5ccccc5)c34)cc2)cc1 ; c1ccc(N2c3cc(-n4c5ccccc5c5ccccc54)ccc3B3c4c2cc(-n2c5ccccc5c5ccccc52)cc4N(c2ccccc2)c2cnn(-c4ccccc4)c23)cc1]

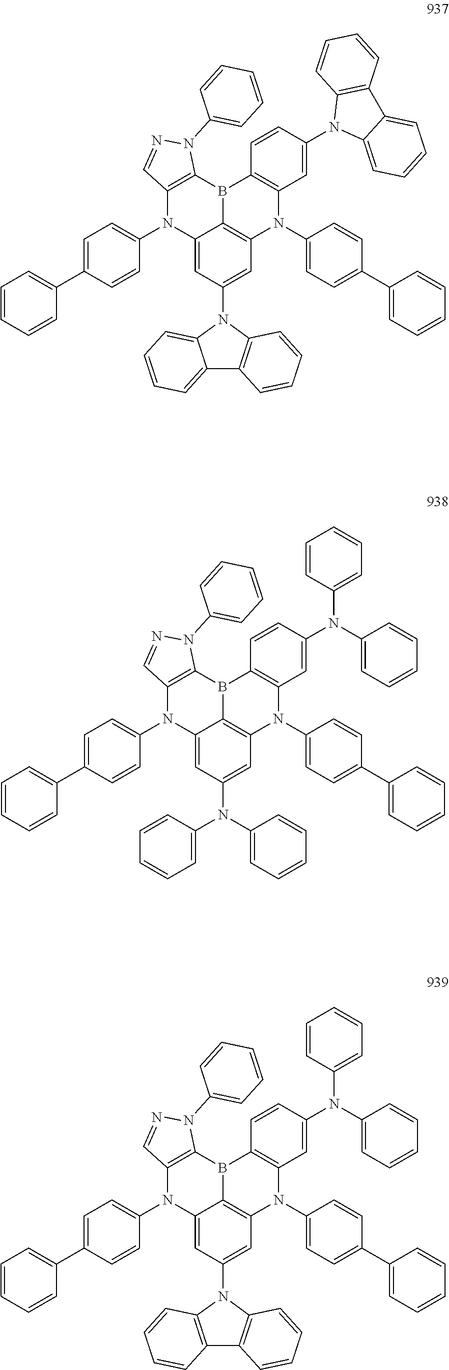 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3cnn(-c5ccccc5)c34)cc2)cc1]

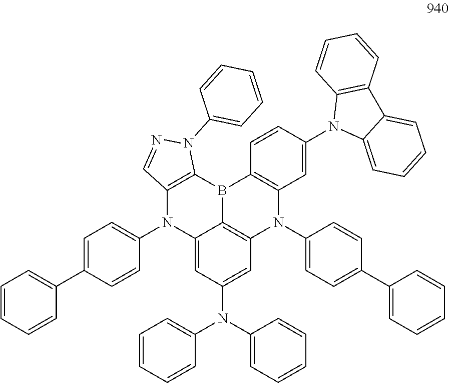 [SMILES: c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3cnn(-c5ccccc5)c34)cc2)cc1]

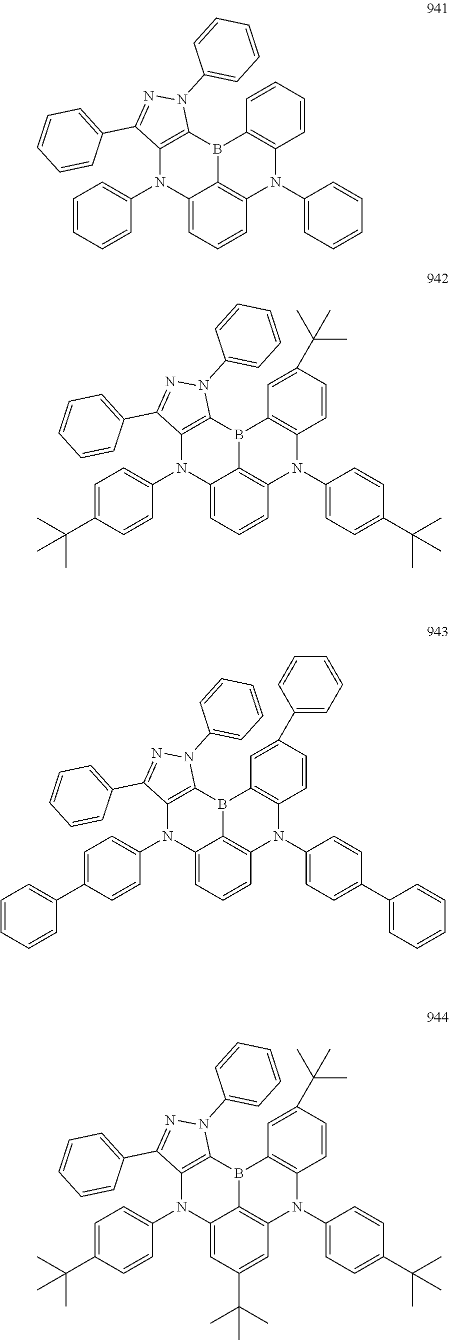

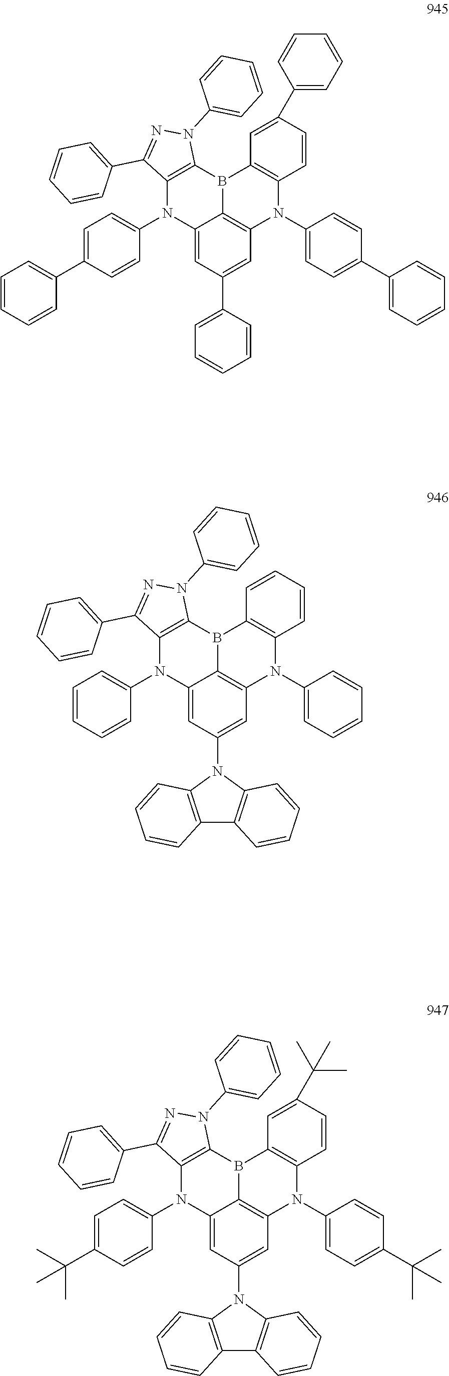 [SMILES: c1ccc(-c2ccc(N3c4ccc(-c5ccccc5)cc4B4c5c3cc(-c3ccccc3)cc5N(c3ccc(-c5ccccc5)cc3)c3c(-c5ccccc5)nn(-c5ccccc5)c34)cc2)cc1 ; c1ccc(-c2nn(-c3ccccc3)c3c2N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2c(-c4ccccc4)nn(-c4ccccc4)c23)cc1]

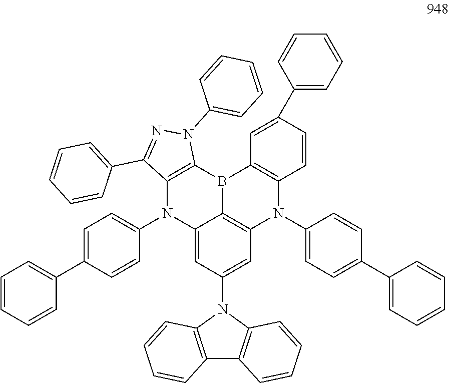 [SMILES: c1ccc(-c2ccc(N3c4ccc(-c5ccccc5)cc4B4c5c3cc(-n3c6ccccc6c6ccccc63)cc5N(c3ccc(-c5ccccc5)cc3)c3c(-c5ccccc5)nn(-c5ccccc5)c34)cc2)cc1]

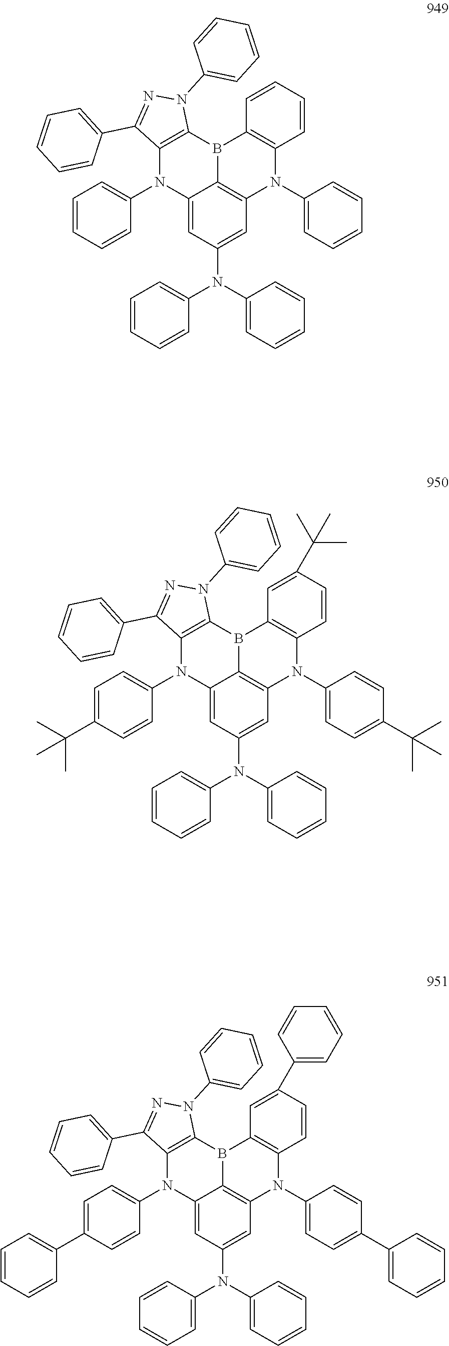 [SMILES: c1ccc(-c2nn(-c3ccccc3)c3c2N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4c2cc(N(c2ccccc2)c2ccccc2)cc4N(c2ccc(C(C)(C)C)cc2)c2c(-c4ccccc4)nn(-c4ccccc4)c23)cc1 ; c1ccc(-c2ccc(N3c4ccc(-c5ccccc5)cc4B4c5c3cc(N(c3ccccc3)c3ccccc3)cc5N(c3ccc(-c5ccccc5)cc3)c3c(-c5ccccc5)nn(-c5ccccc5)c34)cc2)cc1]

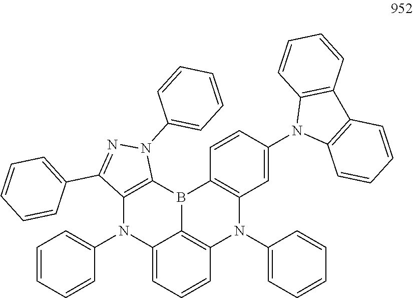 [SMILES: c1ccc(-c2nn(-c3ccccc3)c3c2N(c2ccccc2)c2cccc4c2B3c2ccc(-n3c5ccccc5c5ccccc53)cc2N4c2ccccc2)cc1]

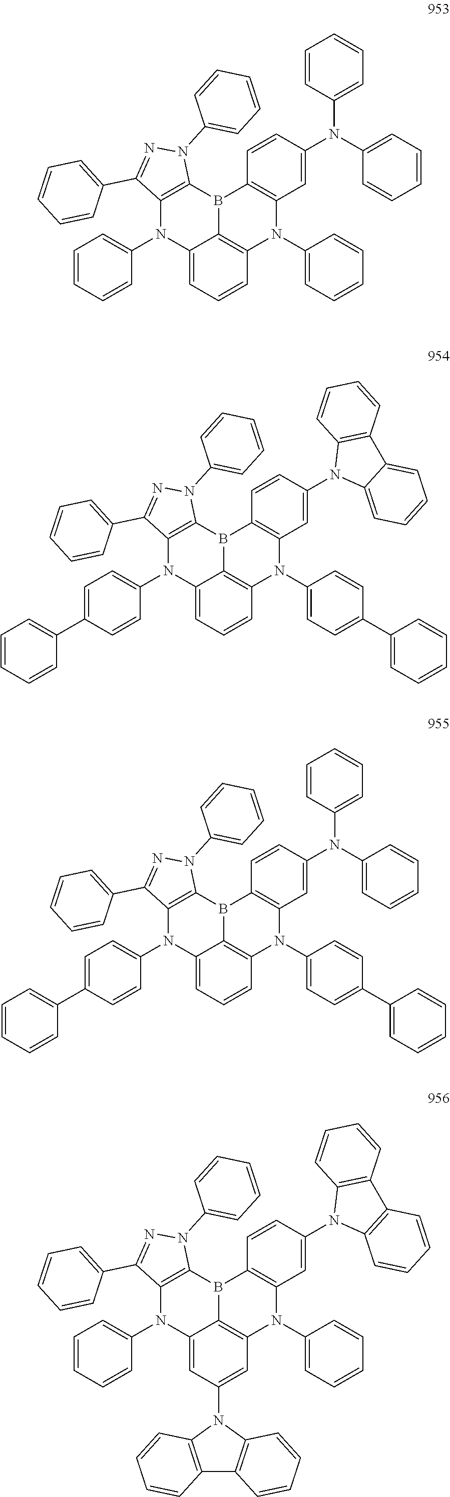 [SMILES: c1ccc(-c2nn(-c3ccccc3)c3c2N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5c3cccc5N(c3ccc(-c5ccccc5)cc3)c3c(-c5ccccc5)nn(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3c(-c5ccccc5)nn(-c5ccccc5)c34)cc2)cc1 ; c1ccc(-c2nn(-c3ccccc3)c3c2N(c2ccccc2)c2cc(-n4c5ccccc5c5ccccc54)cc4c2B3c2ccc(-n3c5ccccc5c5ccccc53)cc2N4c2ccccc2)cc1]

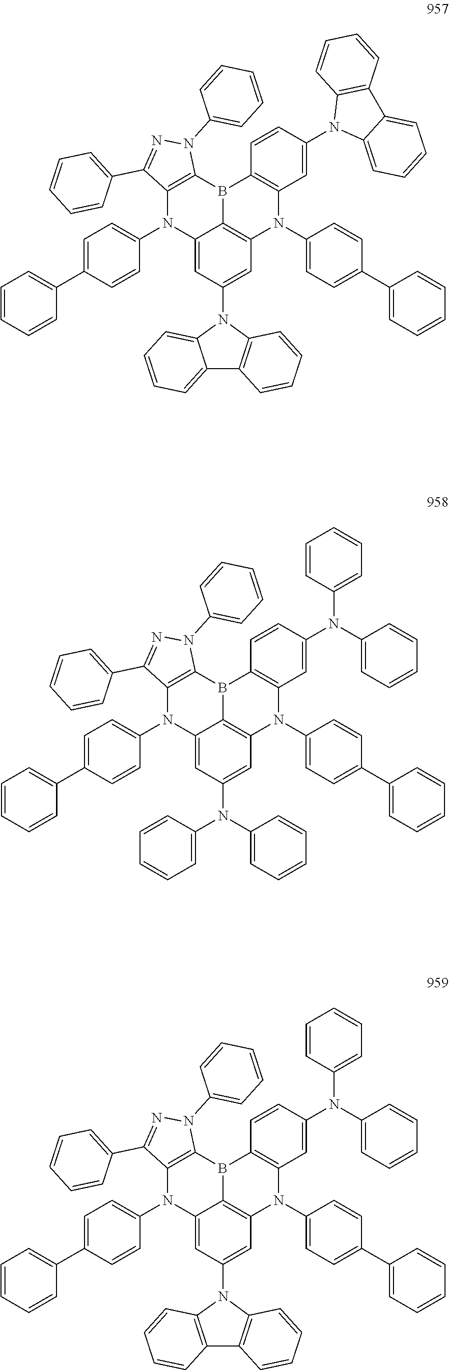 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3c(-c5ccccc5)nn(-c5ccccc5)c34)cc2)cc1 ; c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3c(-c5ccccc5)nn(-c5ccccc5)c34)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3c(-c5ccccc5)nn(-c5ccccc5)c34)cc2)cc1]

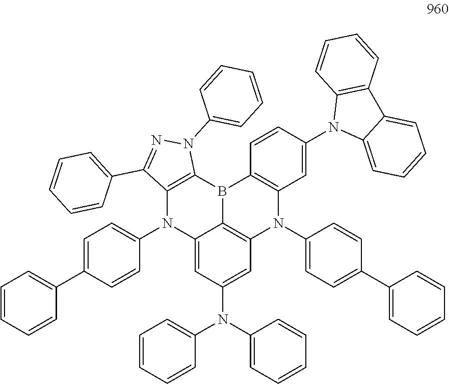 [SMILES: c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3c(-c5ccccc5)nn(-c5ccccc5)c34)cc2)cc1]

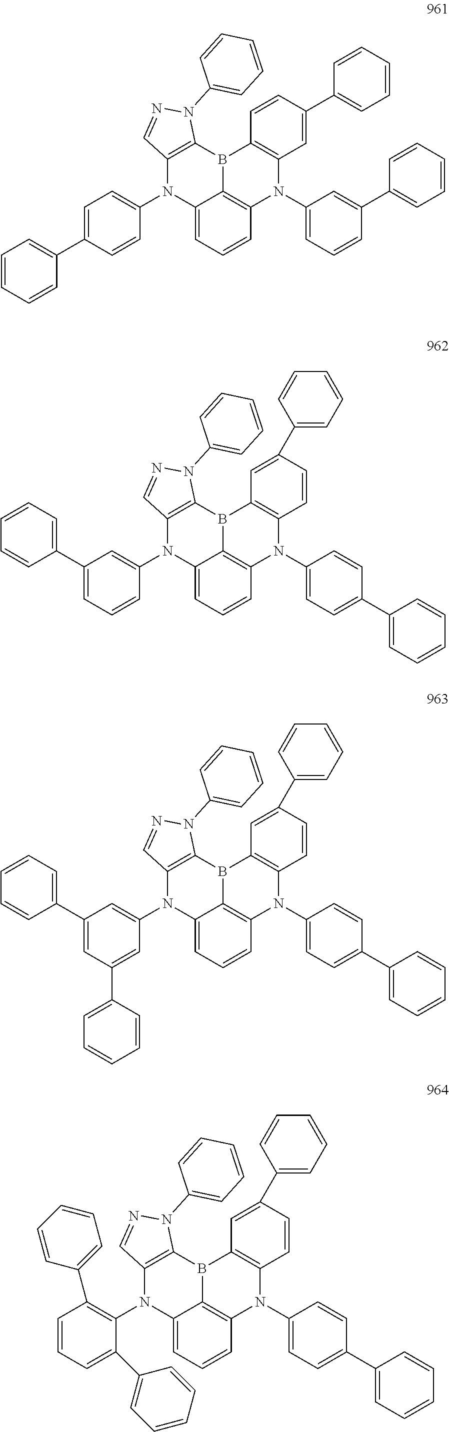 [SMILES: c1ccc(-c2ccc(N3c4cccc5c4B(c4ccc(-c6ccccc6)cc4N5c4cccc(-c5ccccc5)c4)c4c3cnn4-c3ccccc3)cc2)cc1 ; c1ccc(-c2ccc(N3c4ccc(-c5ccccc5)cc4B4c5c3cccc5N(c3cccc(-c5ccccc5)c3)c3cnn(-c5ccccc5)c34)cc2)cc1 ; c1ccc(-c2ccc(N3c4ccc(-c5ccccc5)cc4B4c5c3cccc5N(c3cc(-c5ccccc5)cc(-c5ccccc5)c3)c3cnn(-c5ccccc5)c34)cc2)cc1 ; c1ccc(-c2ccc(N3c4ccc(-c5ccccc5)cc4B4c5c3cccc5N(c3c(-c5ccccc5)cccc3-c3ccccc3)c3cnn(-c5ccccc5)c34)cc2)cc1]

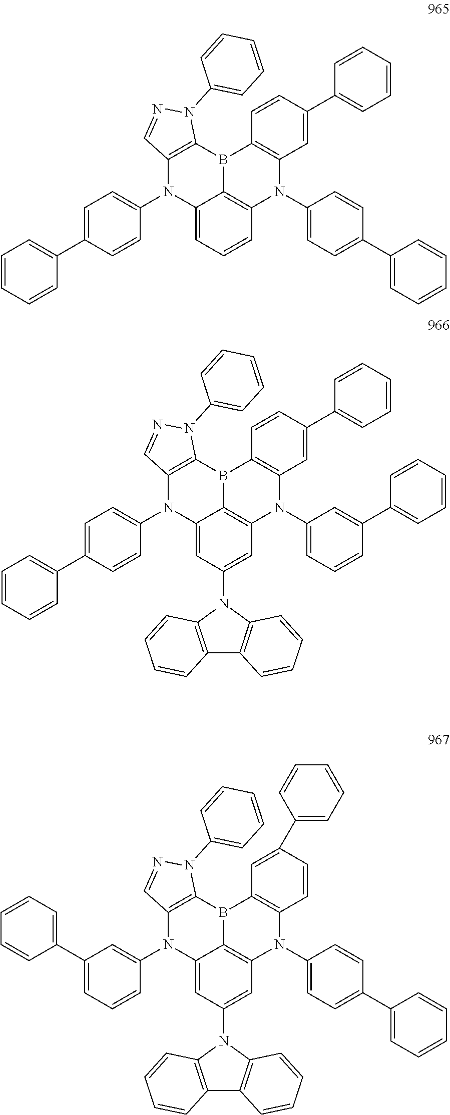 [SMILES: c1ccc(-c2ccc(N3c4cc(-c5ccccc5)ccc4B4c5c3cccc5N(c3ccc(-c5ccccc5)cc3)c3cnn(-c5ccccc5)c34)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c3cnn4-c3ccccc3)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3cnn(-c5ccccc5)c34)cc2)cc1]

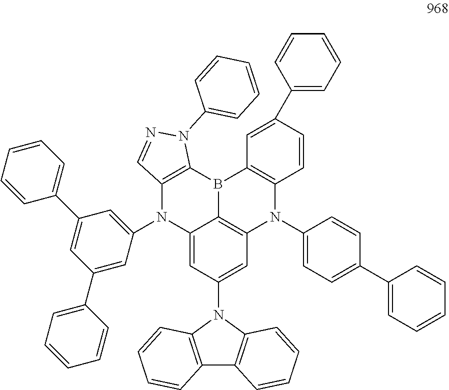 [SMILES: c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3cnn(-c5ccccc5)c34)cc2)cc1]

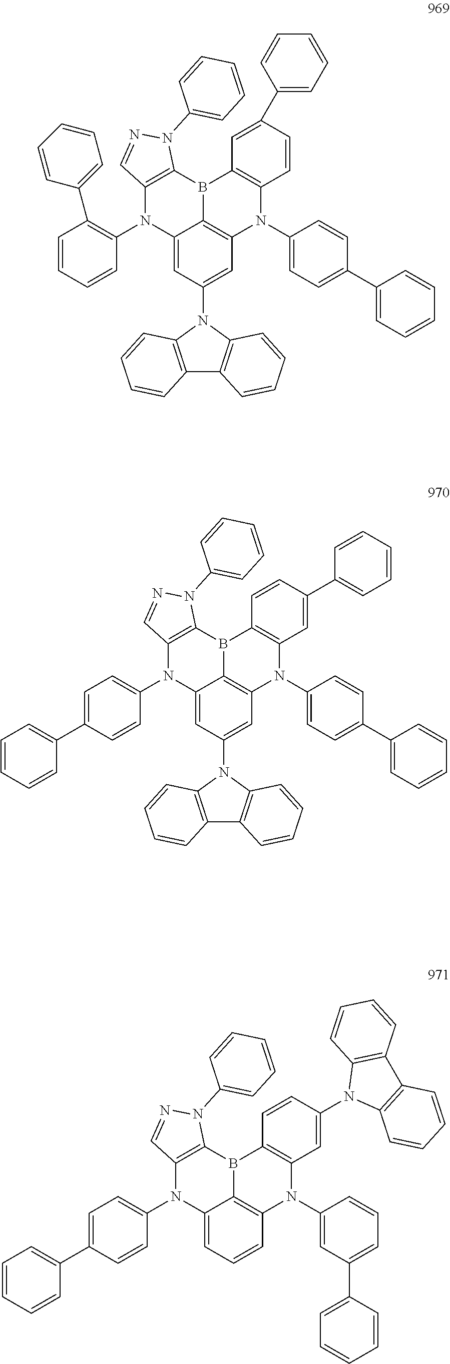 [SMILES: c1ccc(-c2ccc(N3c4ccc(-c5ccccc5)cc4B4c5c3cc(-n3c6ccccc6c6ccccc63)cc5N(c3ccccc3-c3ccccc3)c3cnn(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3cnn(-c5ccccc5)c34)cc2)cc1 ; c1ccc(-c2ccc(N3c4cccc5c4B(c4ccc(-n6c7ccccc7c7ccccc76)cc4N5c4cccc(-c5ccccc5)c4)c4c3cnn4-c3ccccc3)cc2)cc1]

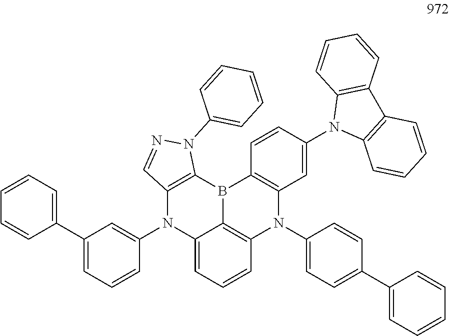 [SMILES: c1ccc(-c2ccc(N3c4cc(-n5c6ccccc6c6ccccc65)ccc4B4c5c3cccc5N(c3cccc(-c5ccccc5)c3)c3cnn(-c5ccccc5)c34)cc2)cc1]

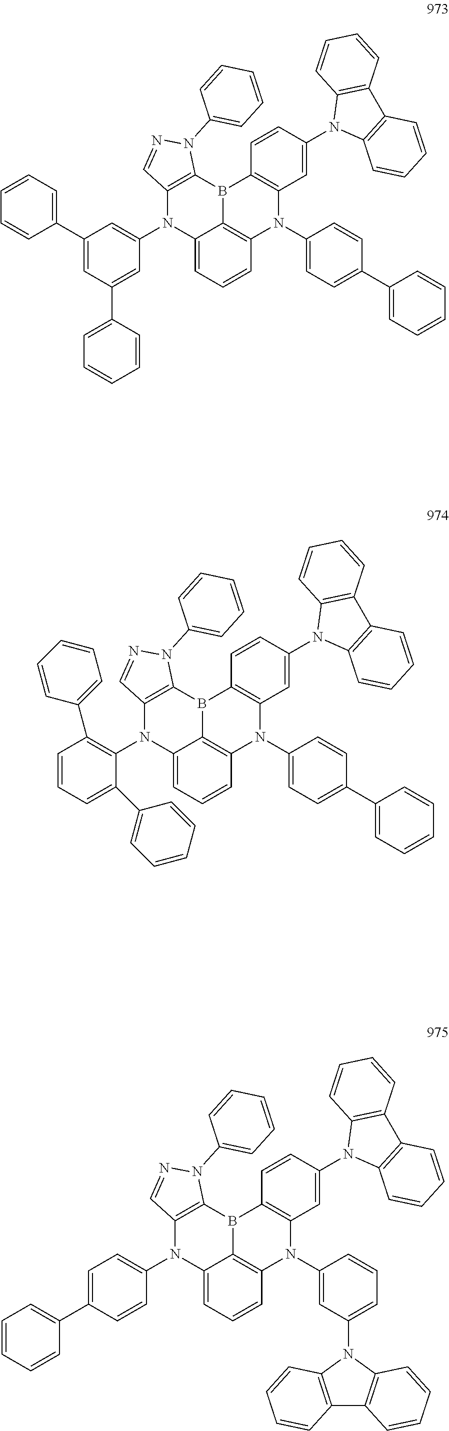 [SMILES: c1ccc(-c2ccc(N3c4cc(-n5c6ccccc6c6ccccc65)ccc4B4c5c3cccc5N(c3cc(-c5ccccc5)cc(-c5ccccc5)c3)c3cnn(-c5ccccc5)c34)cc2)cc1 ; c1ccc(-c2ccc(N3c4cc(-n5c6ccccc6c6ccccc65)ccc4B4c5c3cccc5N(c3c(-c5ccccc5)cccc3-c3ccccc3)c3cnn(-c5ccccc5)c34)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4c3cnn4-c3ccccc3)cc2)cc1]

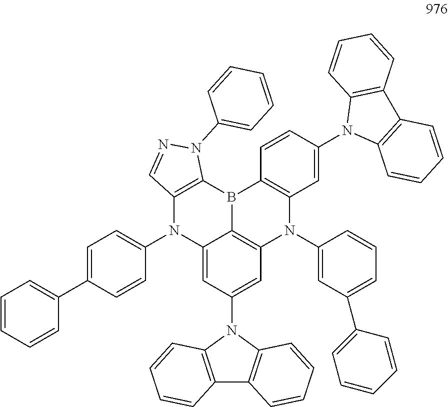 [SMILES: c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3cnn4-c3ccccc3)cc2)cc1]

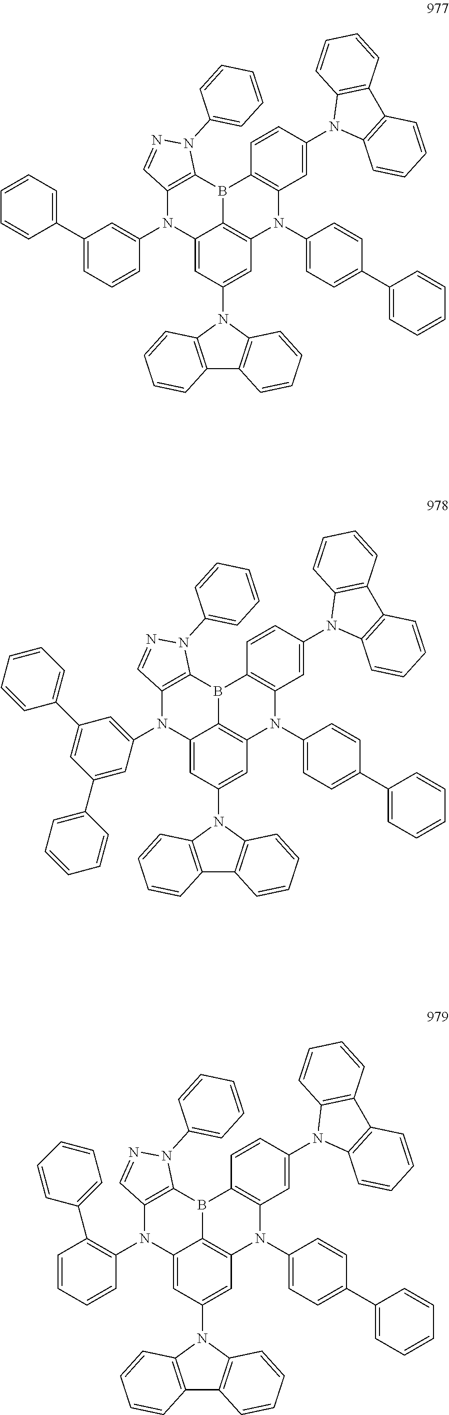 [SMILES: c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3cnn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3cnn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3cnn(-c5ccccc5)c34)cc2)cc1]

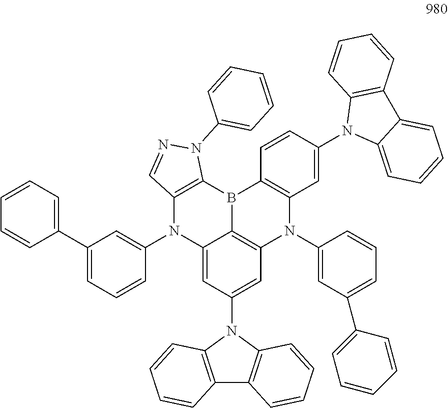 [SMILES: c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3cnn(-c5ccccc5)c34)c2)cc1]

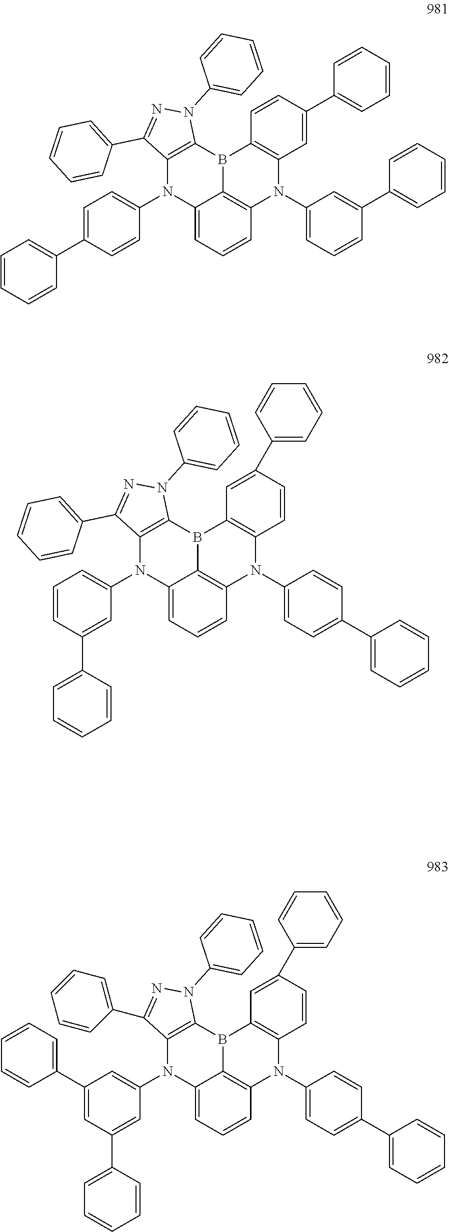 [SMILES: c1ccc(-c2ccc(N3c4cccc5c4B(c4ccc(-c6ccccc6)cc4N5c4cccc(-c5ccccc5)c4)c4c3c(-c3ccccc3)nn4-c3ccccc3)cc2)cc1 ; c1ccc(-c2ccc(N3c4ccc(-c5ccccc5)cc4B4c5c3cccc5N(c3cccc(-c5ccccc5)c3)c3c(-c5ccccc5)nn(-c5ccccc5)c34)cc2)cc1 ; c1ccc(-c2ccc(N3c4ccc(-c5ccccc5)cc4B4c5c3cccc5N(c3cc(-c5ccccc5)cc(-c5ccccc5)c3)c3c(-c5ccccc5)nn(-c5ccccc5)c34)cc2)cc1]

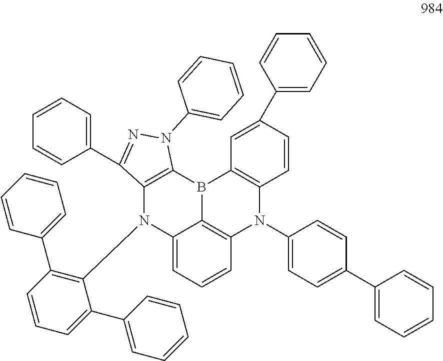 [SMILES: c1ccc(-c2ccc(N3c4ccc(-c5ccccc5)cc4B4c5c3cccc5N(c3c(-c5ccccc5)cccc3-c3ccccc3)c3c(-c5ccccc5)nn(-c5ccccc5)c34)cc2)cc1]

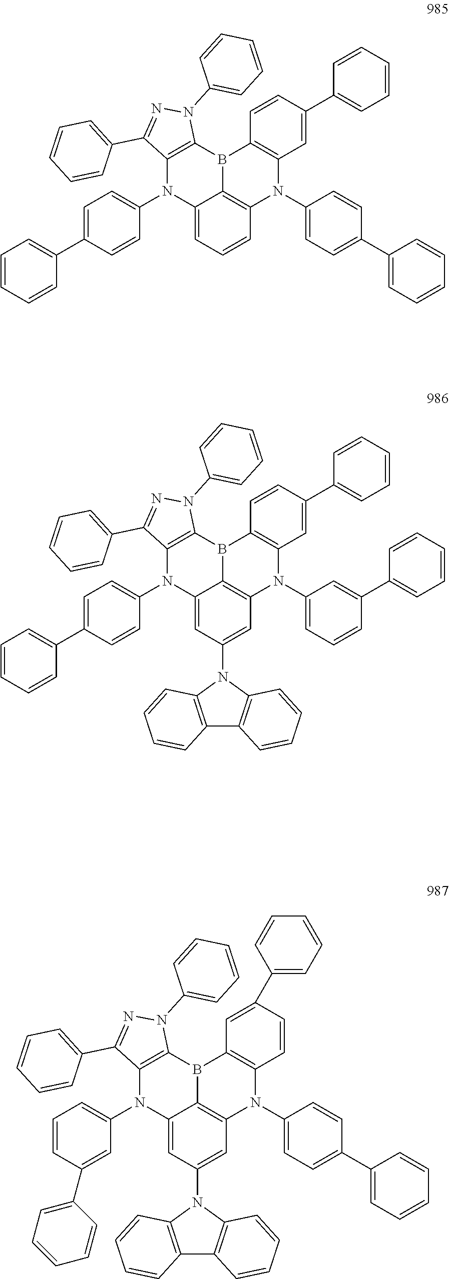 [SMILES: c1ccc(-c2ccc(N3c4cc(-c5ccccc5)ccc4B4c5c3cccc5N(c3ccc(-c5ccccc5)cc3)c3c(-c5ccccc5)nn(-c5ccccc5)c34)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c3c(-c3ccccc3)nn4-c3ccccc3)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3c(-c5ccccc5)nn(-c5ccccc5)c34)cc2)cc1]

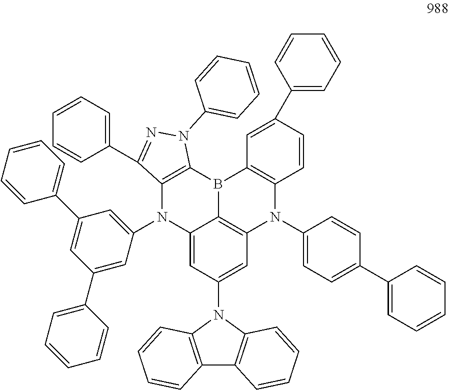 [SMILES: c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3c(-c5ccccc5)nn(-c5ccccc5)c34)cc2)cc1]

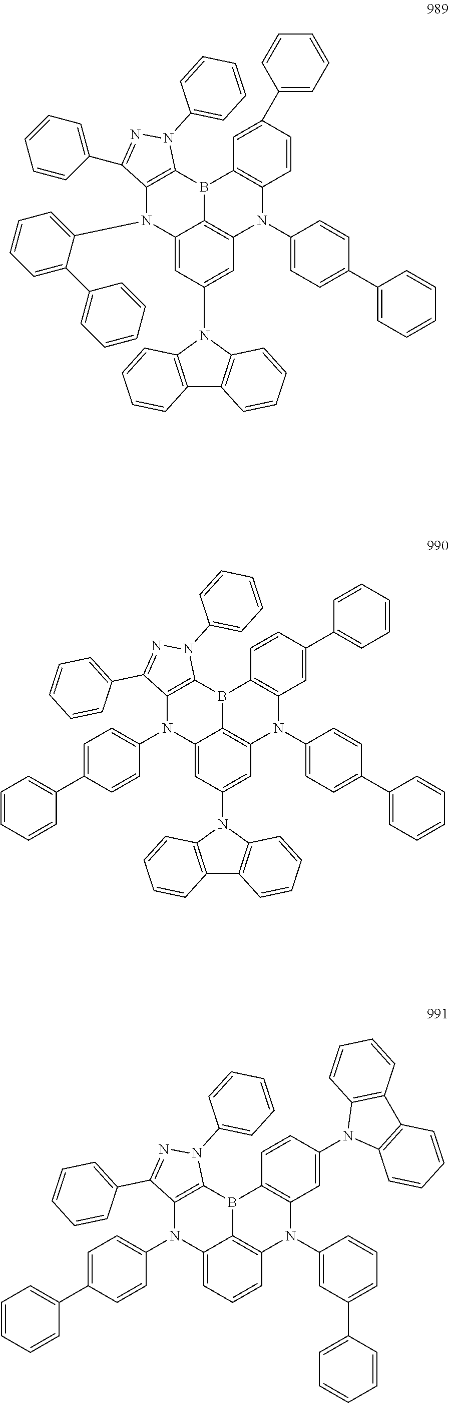 [SMILES: c1ccc(-c2ccc(N3c4ccc(-c5ccccc5)cc4B4c5c3cc(-n3c6ccccc6c6ccccc63)cc5N(c3ccccc3-c3ccccc3)c3c(-c5ccccc5)nn(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3c(-c5ccccc5)nn(-c5ccccc5)c34)cc2)cc1 ; c1ccc(-c2ccc(N3c4cccc5c4B(c4ccc(-n6c7ccccc7c7ccccc76)cc4N5c4cccc(-c5ccccc5)c4)c4c3c(-c3ccccc3)nn4-c3ccccc3)cc2)cc1]

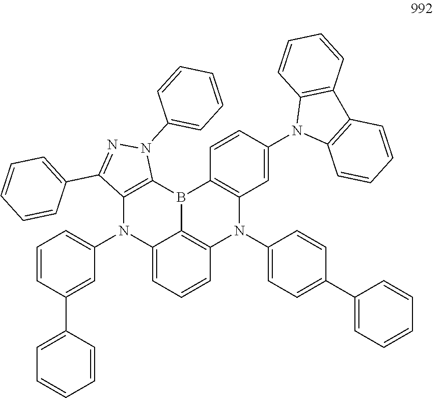 [SMILES: c1ccc(-c2ccc(N3c4cc(-n5c6ccccc6c6ccccc65)ccc4B4c5c3cccc5N(c3cccc(-c5ccccc5)c3)c3c(-c5ccccc5)nn(-c5ccccc5)c34)cc2)cc1]

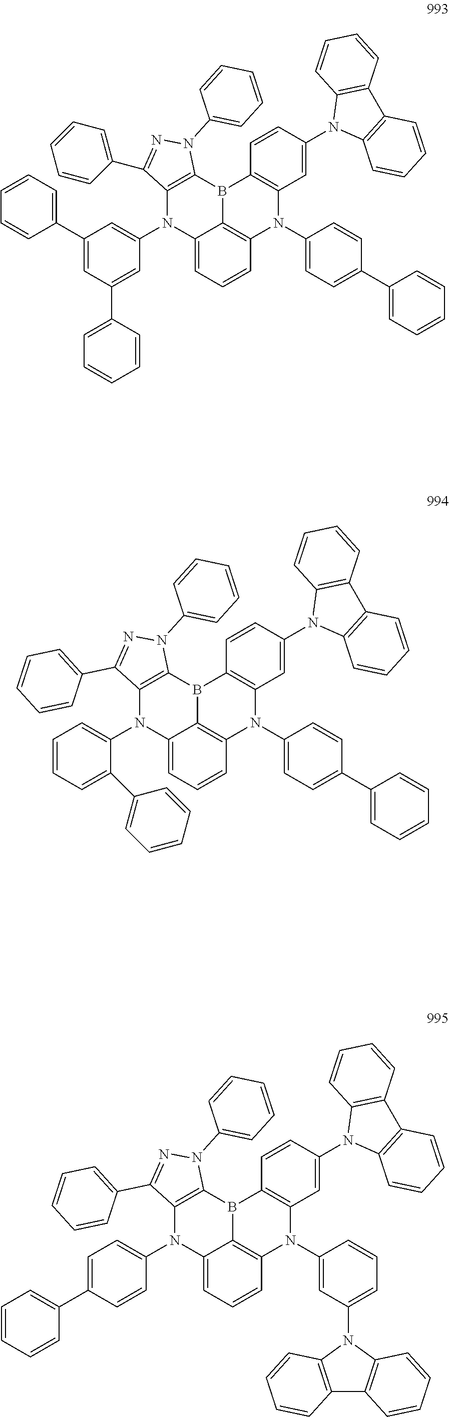 [SMILES: c1ccc(-c2ccc(N3c4cc(-n5c6ccccc6c6ccccc65)ccc4B4c5c3cccc5N(c3cc(-c5ccccc5)cc(-c5ccccc5)c3)c3c(-c5ccccc5)nn(-c5ccccc5)c34)cc2)cc1 ; c1ccc(-c2ccc(N3c4cc(-n5c6ccccc6c6ccccc65)ccc4B4c5c3cccc5N(c3ccccc3-c3ccccc3)c3c(-c5ccccc5)nn(-c5ccccc5)c34)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4c3c(-c3ccccc3)nn4-c3ccccc3)cc2)cc1]

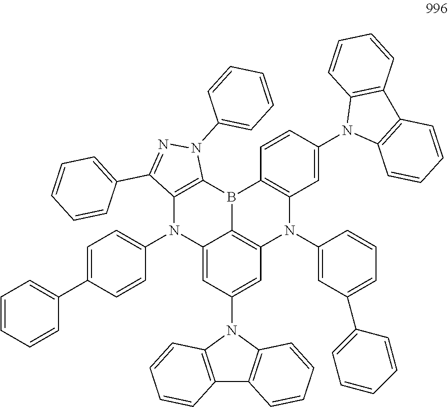 [SMILES: c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3c(-c3ccccc3)nn4-c3ccccc3)cc2)cc1]

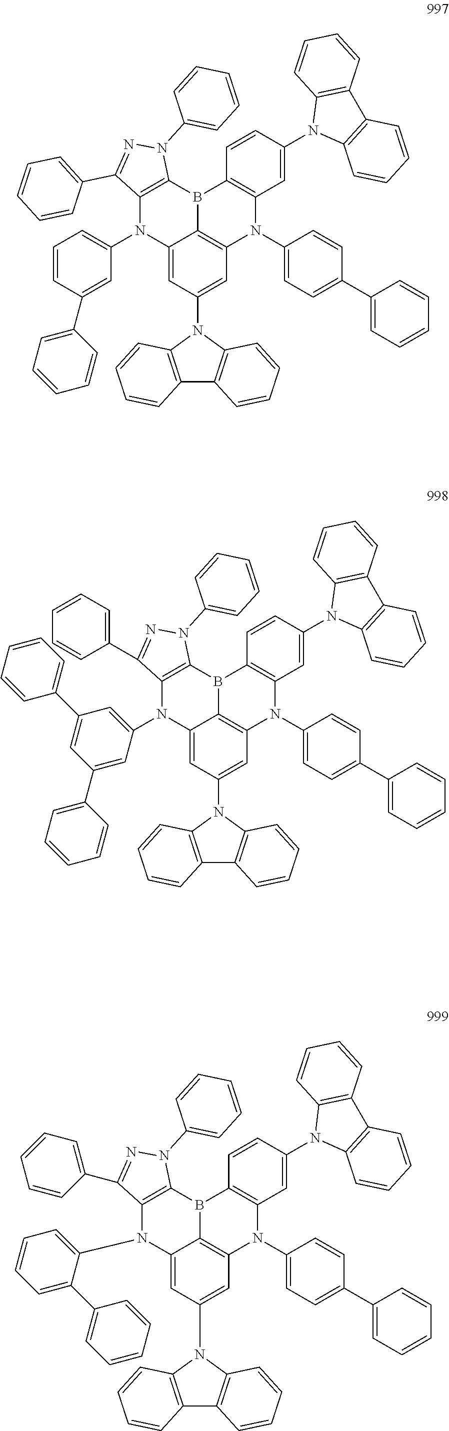 [SMILES: c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3c(-c5ccccc5)nn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3c(-c5ccccc5)nn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3c(-c5ccccc5)nn(-c5ccccc5)c34)cc2)cc1]

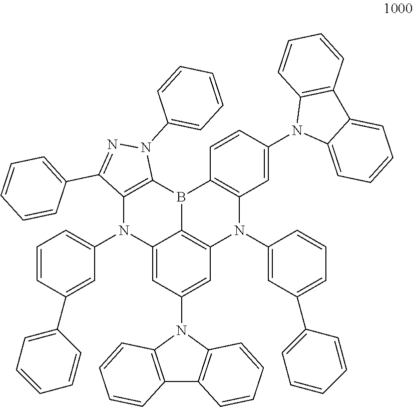 [SMILES: c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3c(-c5ccccc5)nn(-c5ccccc5)c34)c2)cc1]

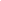

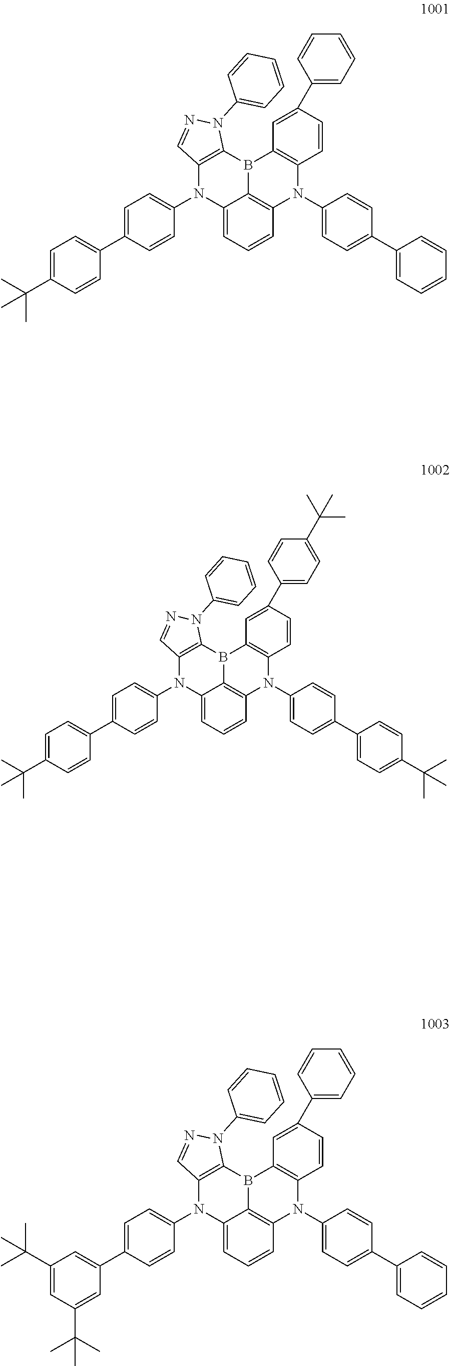

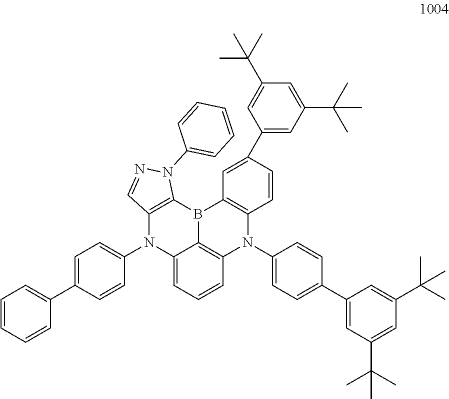 [SMILES: CC(C)(C)c1cc(-c2ccc(N3c4ccc(-c5cc(C(C)(C)C)cc(C(C)(C)C)c5)cc4B4c5c3cccc5N(c3ccc(-c5ccccc5)cc3)c3cnn(-c5ccccc5)c34)cc2)cc(C(C)(C)C)c1]

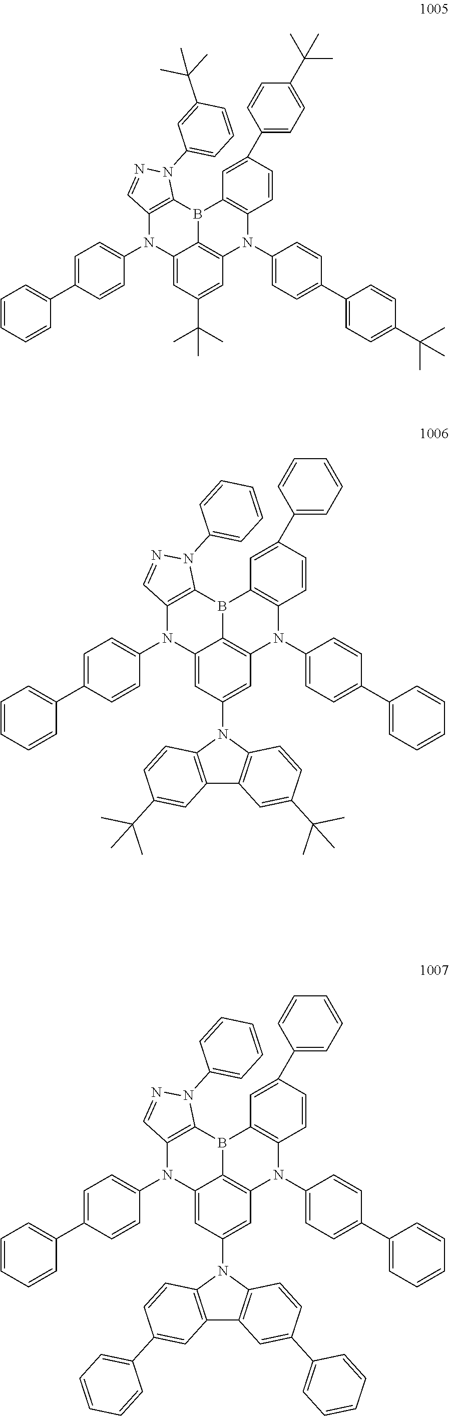 [SMILES: CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3cnn(-c5cccc(C(C)(C)C)c5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1cnn(-c4ccccc4)c1B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3cnn(-c5ccccc5)c34)cc2)cc1]

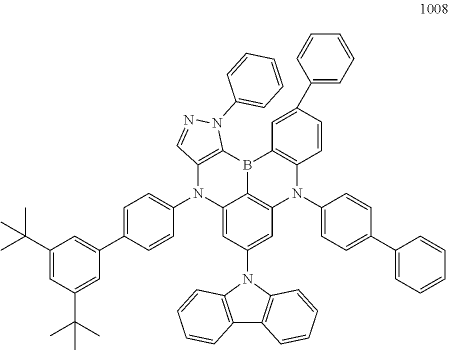 [SMILES: CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4c3cnn4-c3ccccc3)cc2)cc(C(C)(C)C)c1]

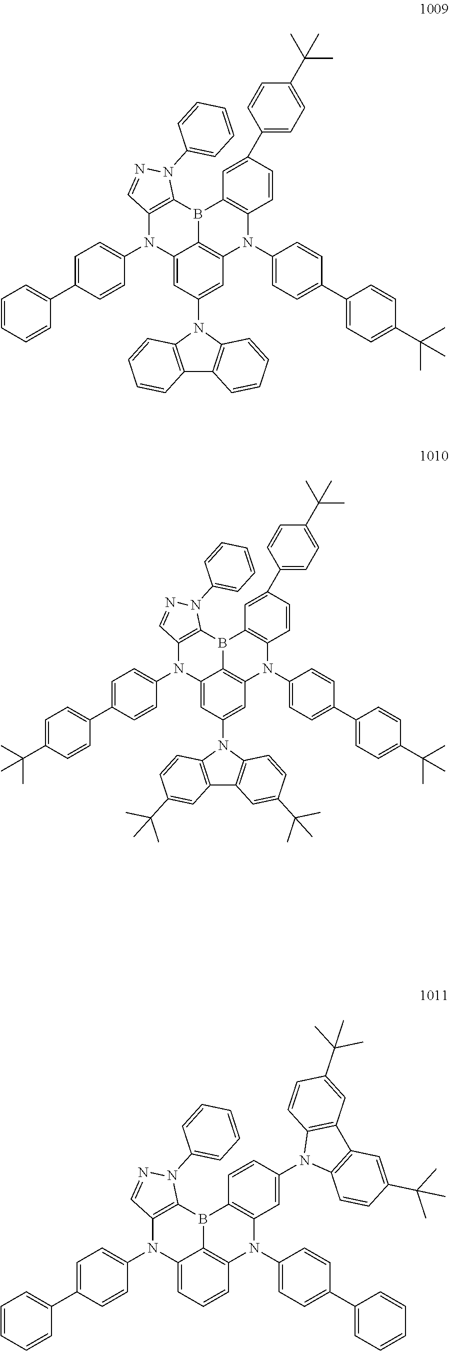

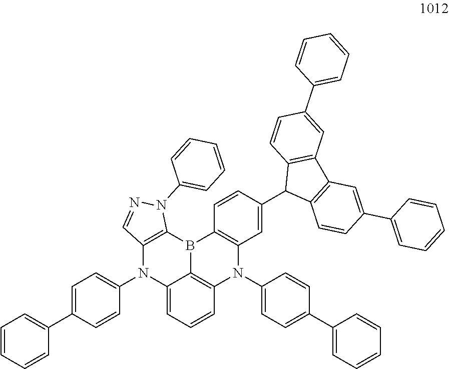 [SMILES: c1ccc(-c2ccc(N3c4cc(C5c6ccc(-c7ccccc7)cc6-c6cc(-c7ccccc7)ccc65)ccc4B4c5c3cccc5N(c3ccc(-c5ccccc5)cc3)c3cnn(-c5ccccc5)c34)cc2)cc1]

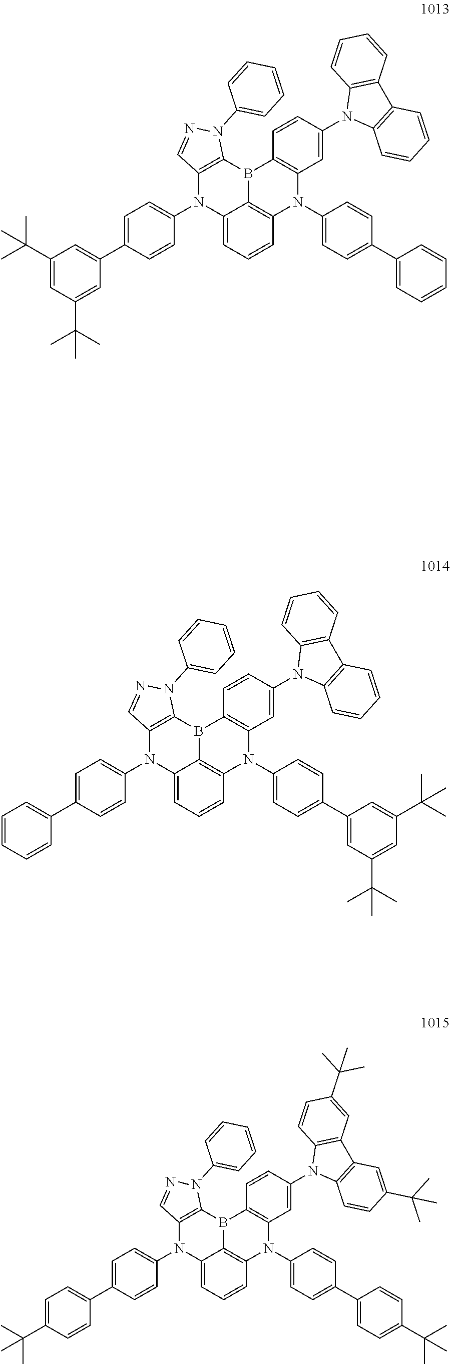

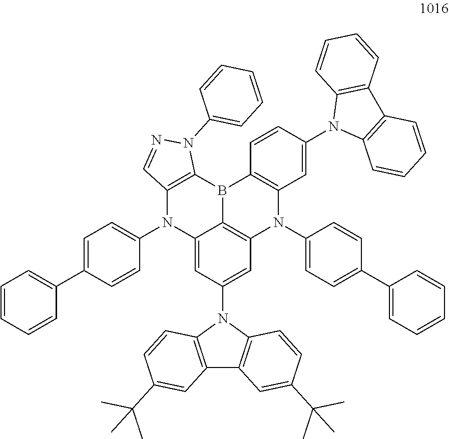 [SMILES: CC(C)(C)c1ccc2c(c1)c1cc(C(C)(C)C)ccc1n2-c1cc2c3c(c1)N(c1ccc(-c4ccccc4)cc1)c1cnn(-c4ccccc4)c1B3c1ccc(-n3c4ccccc4c4ccccc43)cc1N2c1ccc(-c2ccccc2)cc1]

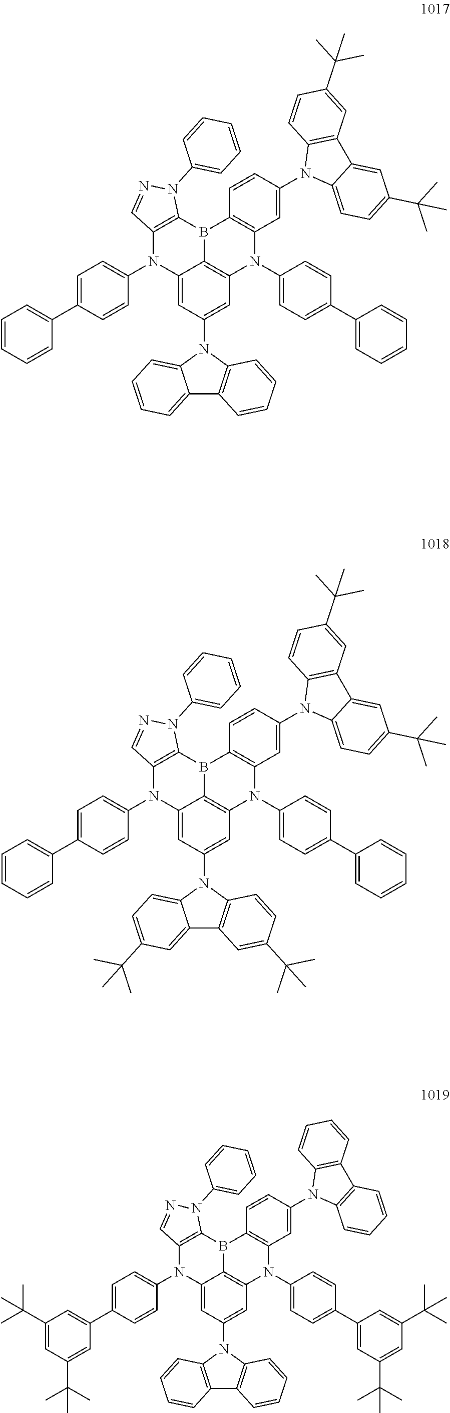

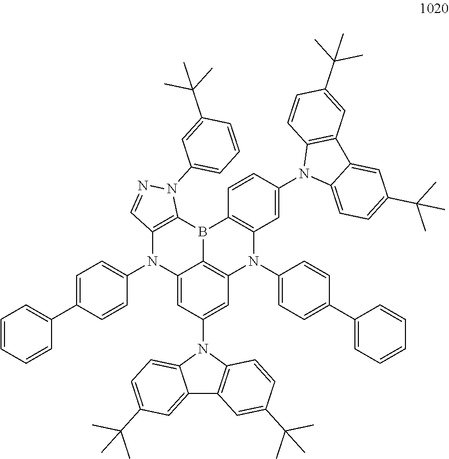 [SMILES: CC(C)(C)c1cccc(-n2ncc3c2B2c4ccc(-n5c6ccc(C(C)(C)C)cc6c6cc(C(C)(C)C)ccc65)cc4N(c4ccc(-c5ccccc5)cc4)c4cc(-n5c6ccc(C(C)(C)C)cc6c6cc(C(C)(C)C)ccc65)cc(c42)N3c2ccc(-c3ccccc3)cc2)c1]

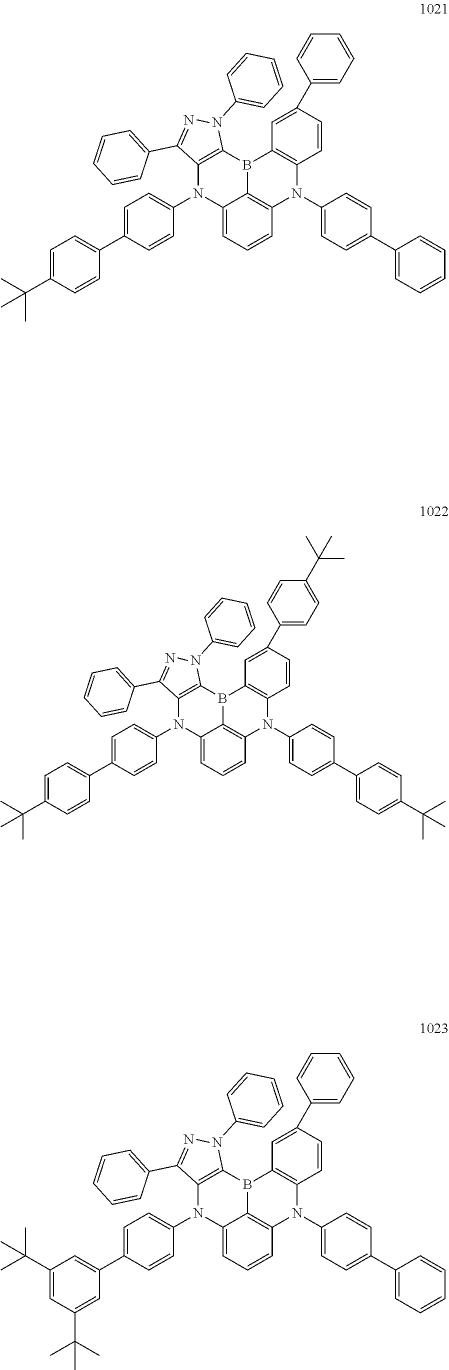

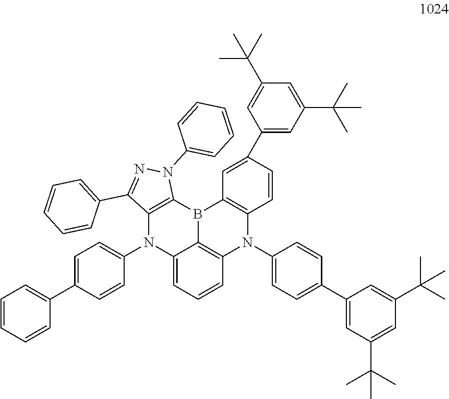 [SMILES: CC(C)(C)c1cc(-c2ccc(N3c4ccc(-c5cc(C(C)(C)C)cc(C(C)(C)C)c5)cc4B4c5c3cccc5N(c3ccc(-c5ccccc5)cc3)c3c(-c5ccccc5)nn(-c5ccccc5)c34)cc2)cc(C(C)(C)C)c1]

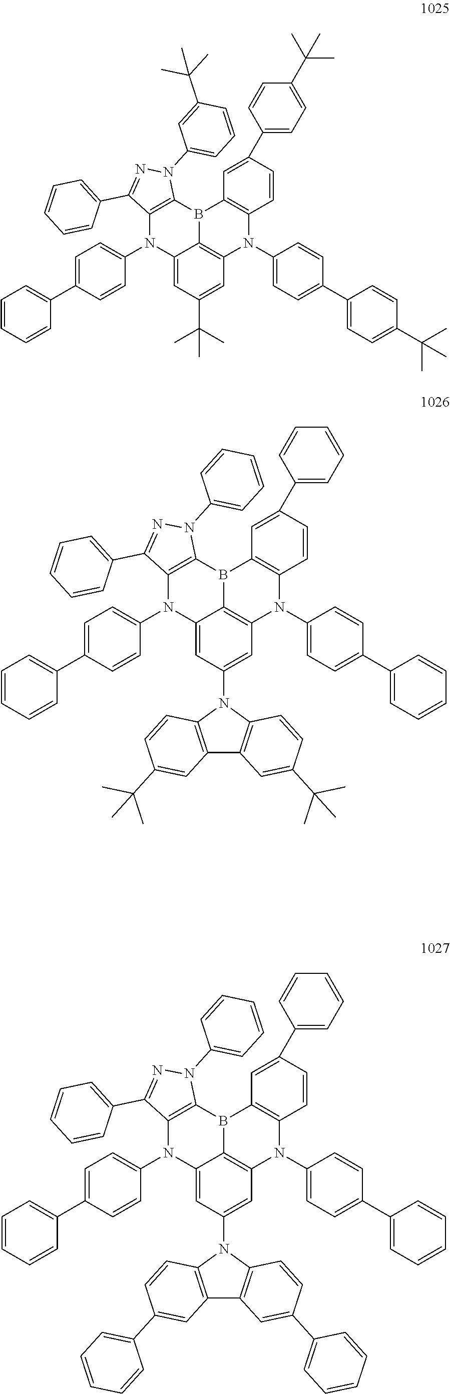 [SMILES: CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3c(-c5ccccc5)nn(-c5cccc(C(C)(C)C)c5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1c(-c4ccccc4)nn(-c4ccccc4)c1B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3c(-c5ccccc5)nn(-c5ccccc5)c34)cc2)cc1]

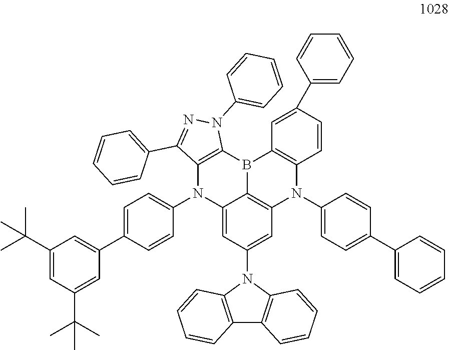 [SMILES: CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4c3c(-c3ccccc3)nn4-c3ccccc3)cc2)cc(C(C)(C)C)c1]

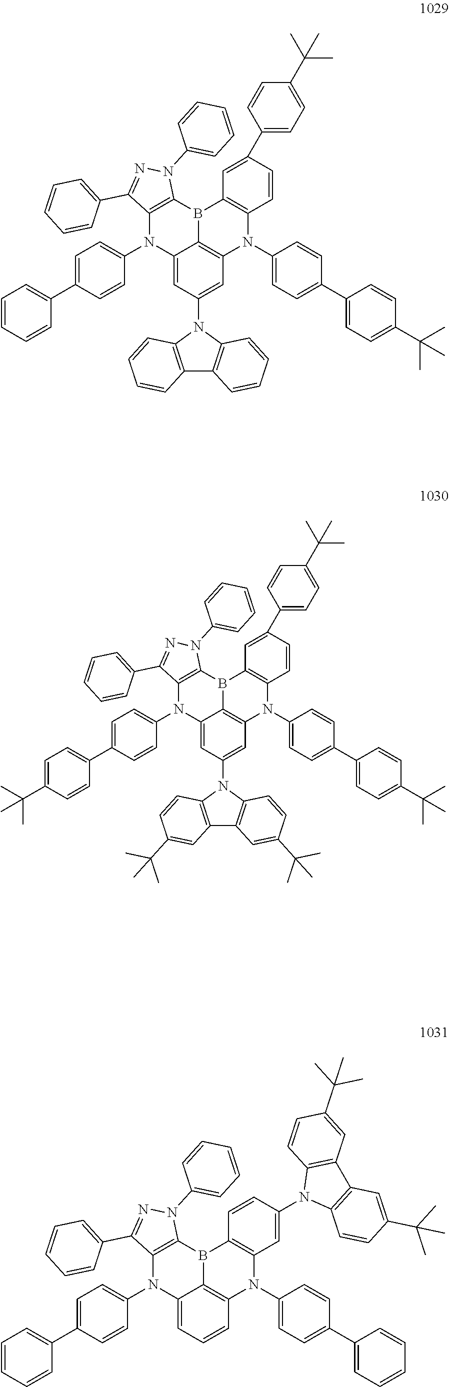

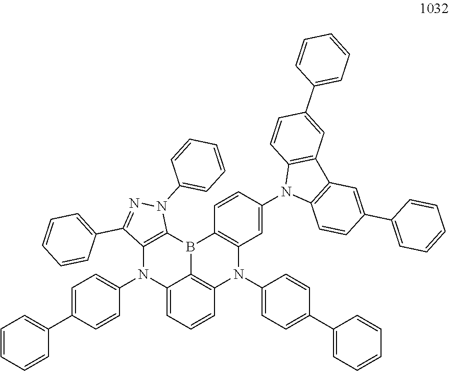 [SMILES: c1ccc(-c2ccc(N3c4cc(-n5c6ccc(-c7ccccc7)cc6c6cc(-c7ccccc7)ccc65)ccc4B4c5c3cccc5N(c3ccc(-c5ccccc5)cc3)c3c(-c5ccccc5)nn(-c5ccccc5)c34)cc2)cc1]

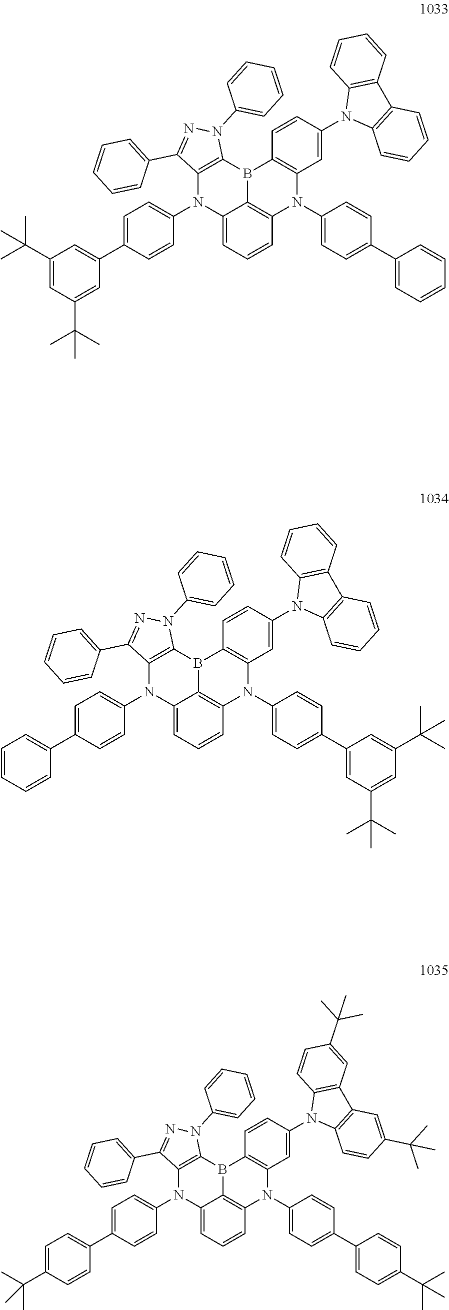

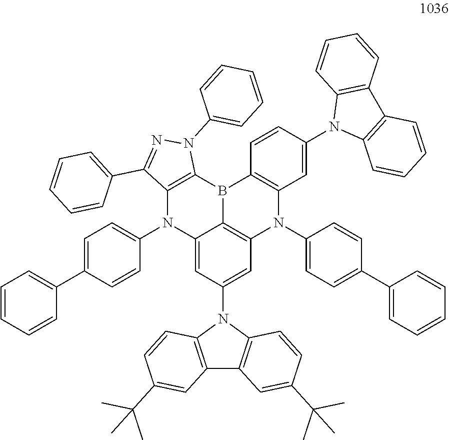 [SMILES: CC(C)(C)c1ccc2c(c1)c1cc(C(C)(C)C)ccc1n2-c1cc2c3c(c1)N(c1ccc(-c4ccccc4)cc1)c1c(-c4ccccc4)nn(-c4ccccc4)c1B3c1ccc(-n3c4ccccc4c4ccccc43)cc1N2c1ccc(-c2ccccc2)cc1]

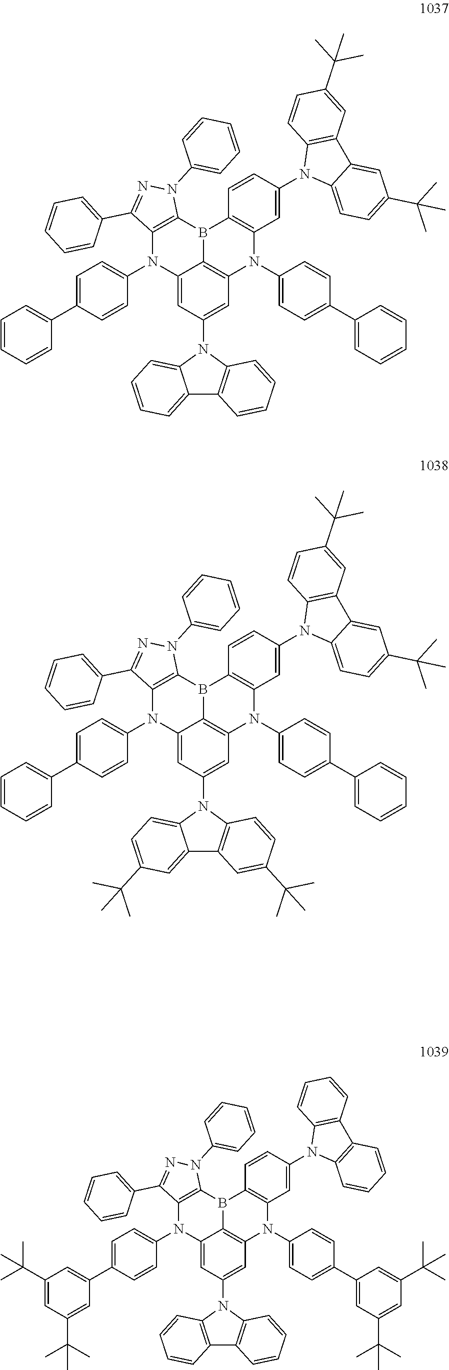

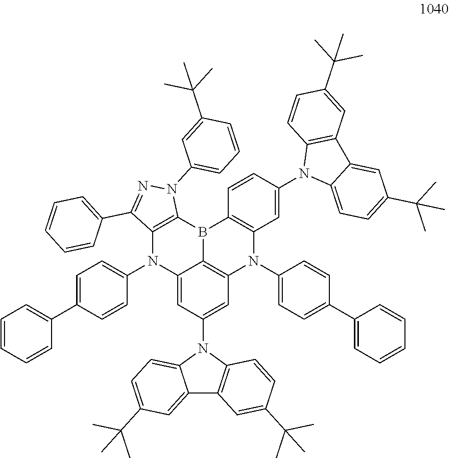 [SMILES: CC(C)(C)c1cccc(-n2nc(-c3ccccc3)c3c2B2c4ccc(-n5c6ccc(C(C)(C)C)cc6c6cc(C(C)(C)C)ccc65)cc4N(c4ccc(-c5ccccc5)cc4)c4cc(-n5c6ccc(C(C)(C)C)cc6c6cc(C(C)(C)C)ccc65)cc(c42)N3c2ccc(-c3ccccc3)cc2)c1]

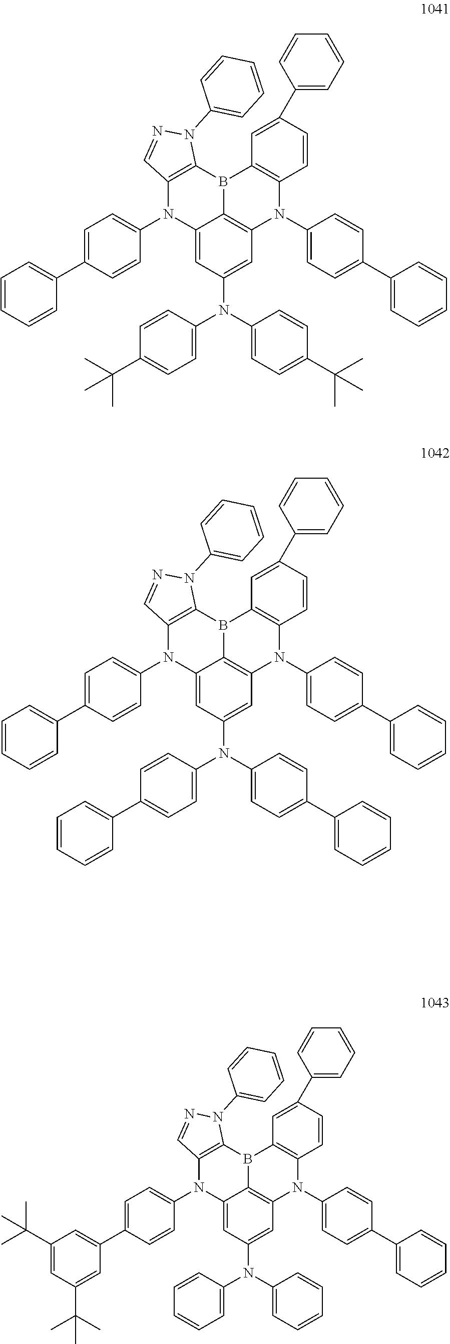 [SMILES: CC(C)(C)c1ccc(N(c2ccc(C(C)(C)C)cc2)c2cc3c4c(c2)N(c2ccc(-c5ccccc5)cc2)c2cnn(-c5ccccc5)c2B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3cnn(-c6ccccc6)c3B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c3cnn4-c3ccccc3)cc2)cc(C(C)(C)C)c1]

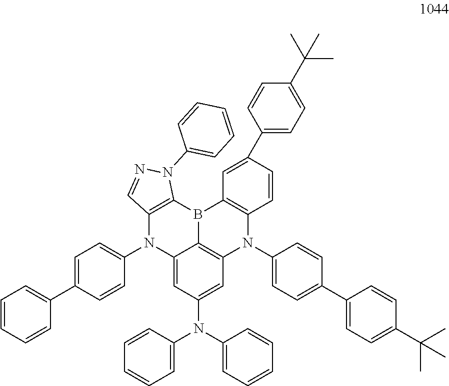 [SMILES: CC(C)(C)c1ccc(-c2ccc(N3c4ccc(-c5ccc(C(C)(C)C)cc5)cc4B4c5c3cc(N(c3ccccc3)c3ccccc3)cc5N(c3ccc(-c5ccccc5)cc3)c3cnn(-c5ccccc5)c34)cc2)cc1]

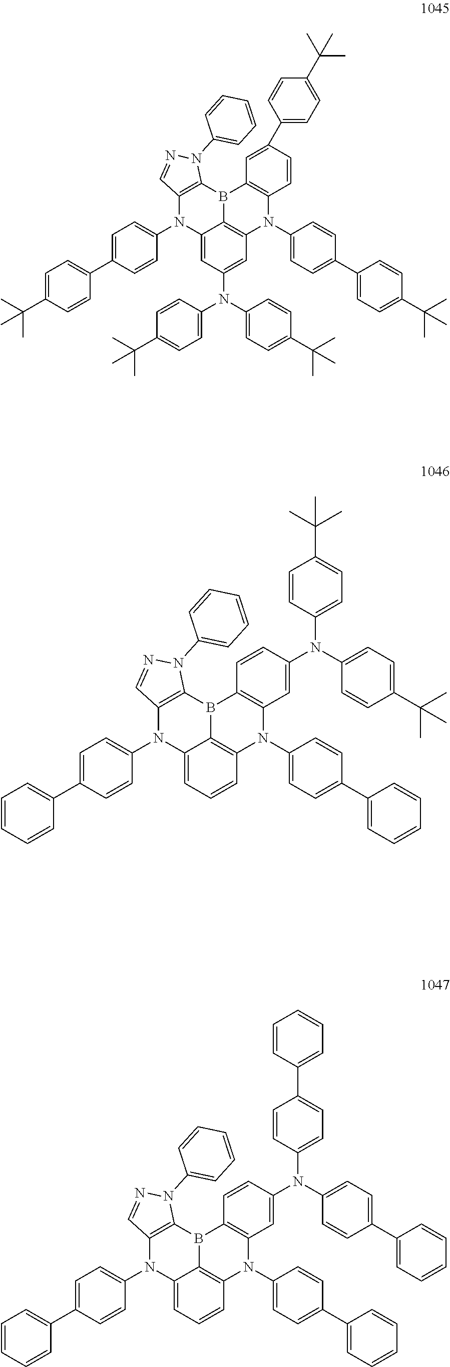 [SMILES: CC(C)(C)c1ccc(-c2ccc(N3c4ccc(-c5ccc(C(C)(C)C)cc5)cc4B4c5c3cc(N(c3ccc(C(C)(C)C)cc3)c3ccc(C(C)(C)C)cc3)cc5N(c3ccc(-c5ccc(C(C)(C)C)cc5)cc3)c3cnn(-c5ccccc5)c34)cc2)cc1 ; CC(C)(C)c1ccc(N(c2ccc(C(C)(C)C)cc2)c2ccc3c(c2)N(c2ccc(-c4ccccc4)cc2)c2cccc4c2B3c2c(cnn2-c2ccccc2)N4c2ccc(-c3ccccc3)cc2)cc1 ; c1ccc(-c2ccc(N(c3ccc(-c4ccccc4)cc3)c3ccc4c(c3)N(c3ccc(-c5ccccc5)cc3)c3cccc5c3B4c3c(cnn3-c3ccccc3)N5c3ccc(-c4ccccc4)cc3)cc2)cc1]

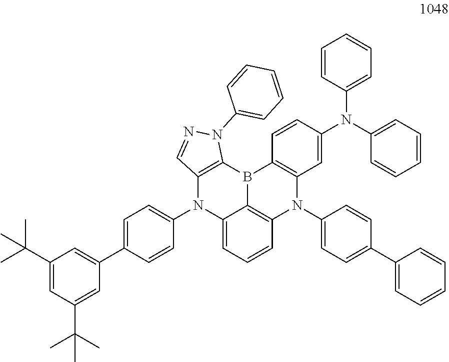 [SMILES: CC(C)(C)c1cc(-c2ccc(N3c4cccc5c4B(c4ccc(N(c6ccccc6)c6ccccc6)cc4N5c4ccc(-c5ccccc5)cc4)c4c3cnn4-c3ccccc3)cc2)cc(C(C)(C)C)c1]

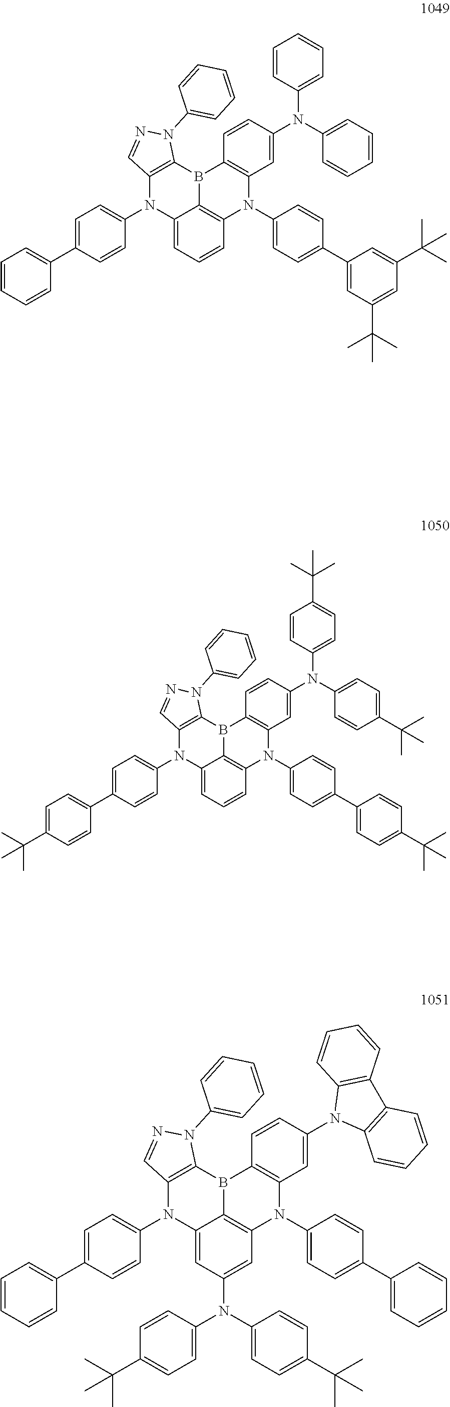

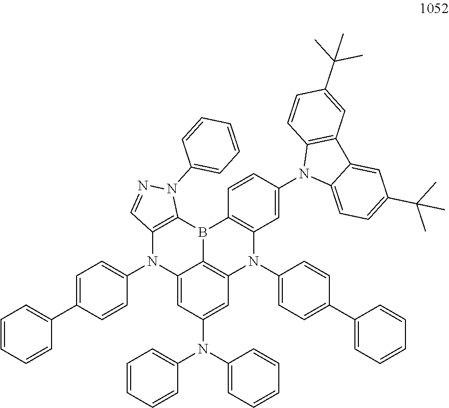 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(cnn1-c1ccccc1)N3c1ccc(-c2ccccc2)cc1]

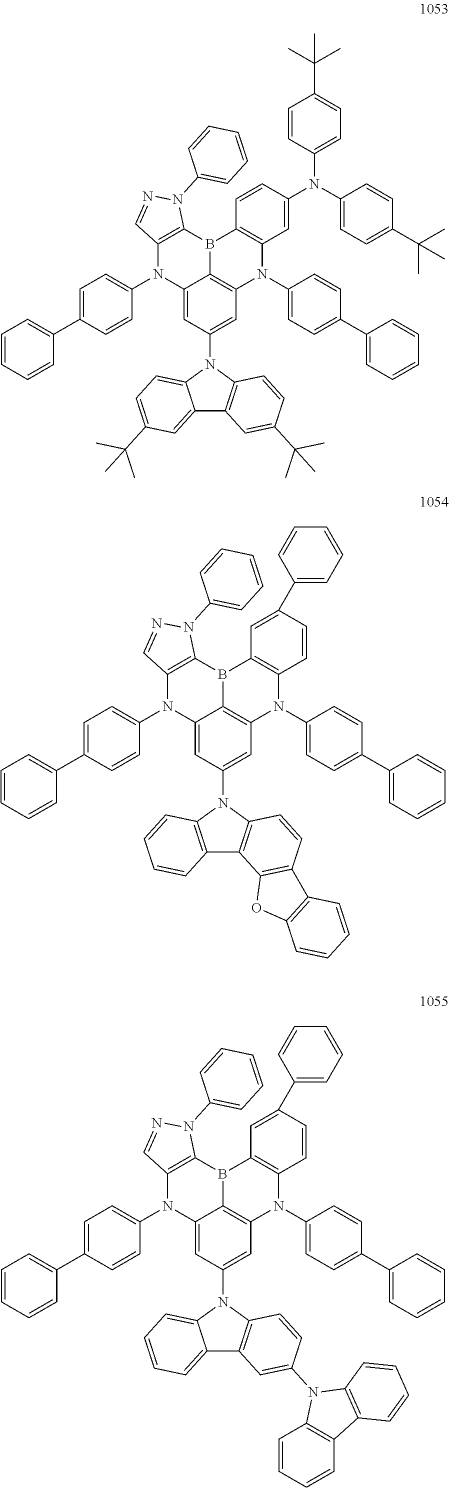 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2c(cnn2-c2ccccc2)N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3cnn(-c5ccccc5)c34)cc2)cc1 ; c1ccc(-c2ccc(N3c4ccc(-c5ccccc5)cc4B4c5c3cc(-n3c6ccccc6c6cc(-n7c8ccccc8c8ccccc87)ccc63)cc5N(c3ccc(-c5ccccc5)cc3)c3cnn(-c5ccccc5)c34)cc2)cc1]

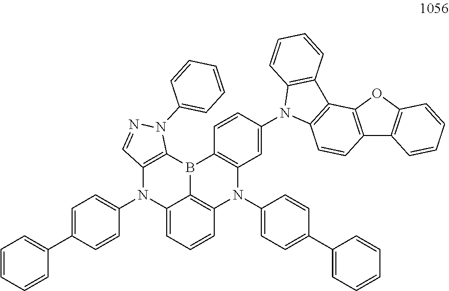 [SMILES: c1ccc(-c2ccc(N3c4cc(-n5c6ccccc6c6c7oc8ccccc8c7ccc65)ccc4B4c5c3cccc5N(c3ccc(-c5ccccc5)cc3)c3cnn(-c5ccccc5)c34)cc2)cc1]

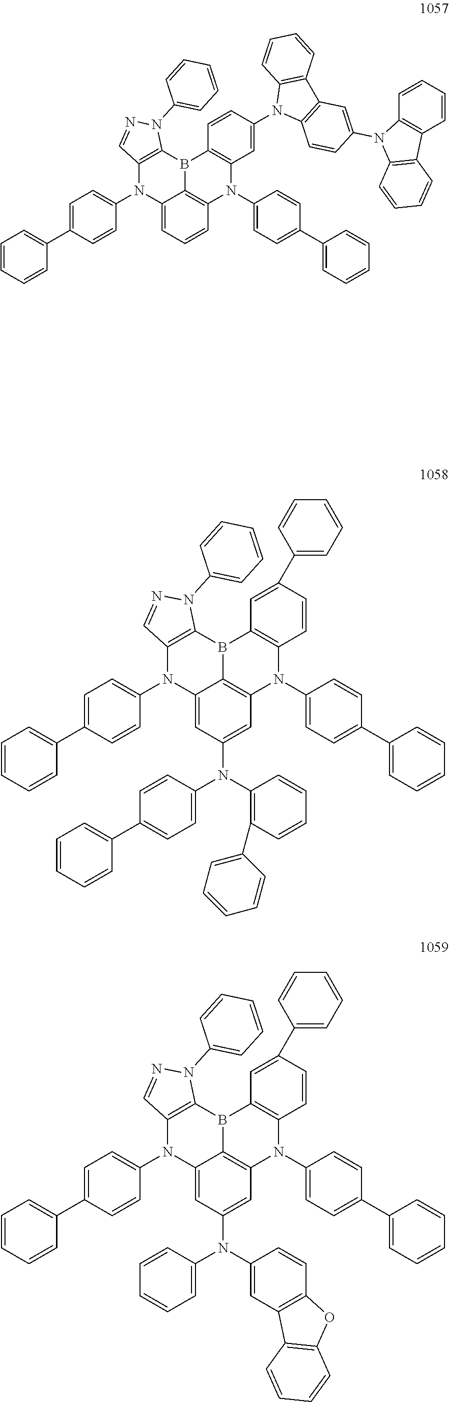 [SMILES: c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3cnn(-c5ccccc5)c34)cc2)cc1 ; c1ccc(-c2ccc(N(c3cc4c5c(c3)N(c3ccc(-c6ccccc6)cc3)c3cnn(-c6ccccc6)c3B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3cnn(-c5ccccc5)c34)cc2)cc1]

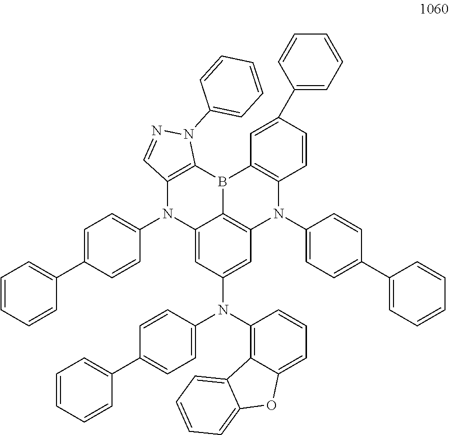 [SMILES: c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3cnn(-c5ccccc5)c34)cc2)cc1]

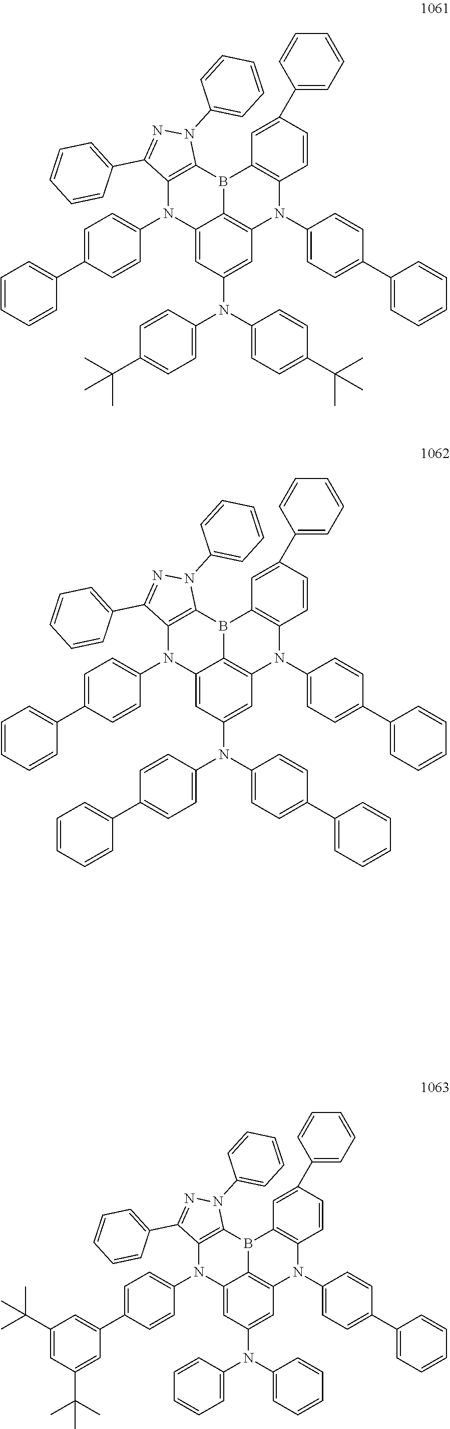 [SMILES: CC(C)(C)c1ccc(N(c2ccc(C(C)(C)C)cc2)c2cc3c4c(c2)N(c2ccc(-c5ccccc5)cc2)c2c(-c5ccccc5)nn(-c5ccccc5)c2B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(-c6ccccc6)nn(-c6ccccc6)c3B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c3c(-c3ccccc3)nn4-c3ccccc3)cc2)cc(C(C)(C)C)c1]

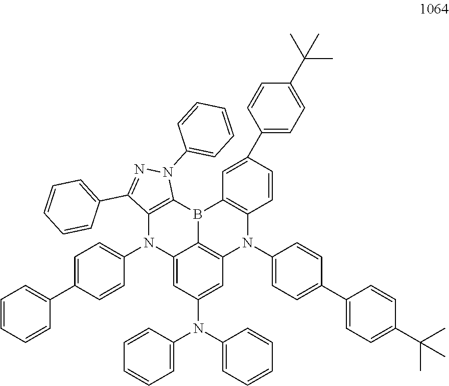 [SMILES: CC(C)(C)c1ccc(-c2ccc(N3c4ccc(-c5ccc(C(C)(C)C)cc5)cc4B4c5c3cc(N(c3ccccc3)c3ccccc3)cc5N(c3ccc(-c5ccccc5)cc3)c3c(-c5ccccc5)nn(-c5ccccc5)c34)cc2)cc1]

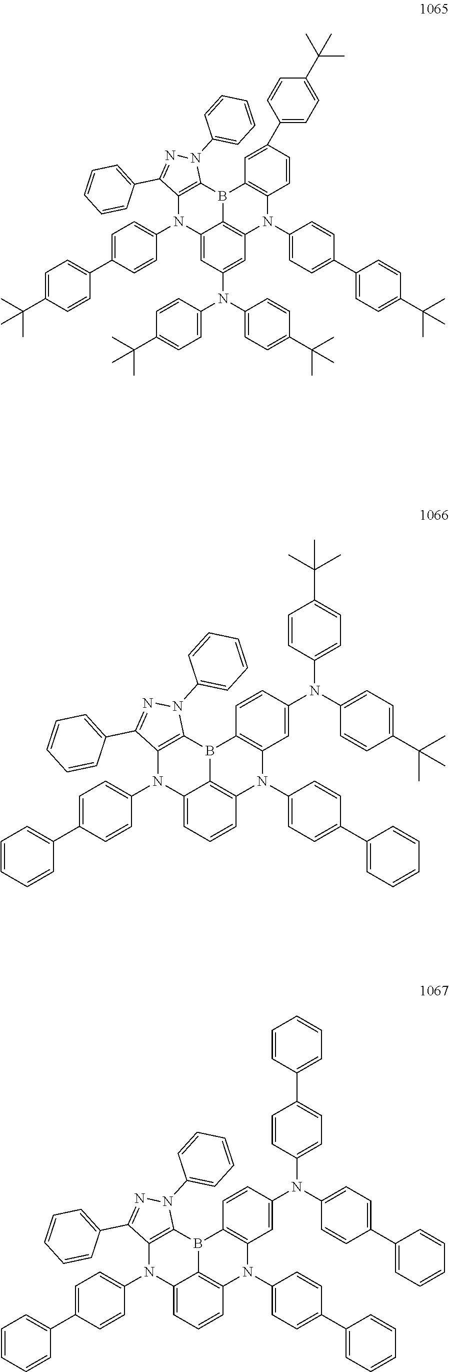 [SMILES: CC(C)(C)c1ccc(-c2ccc(N3c4ccc(-c5ccc(C(C)(C)C)cc5)cc4B4c5c3cc(N(c3ccc(C(C)(C)C)cc3)c3ccc(C(C)(C)C)cc3)cc5N(c3ccc(-c5ccc(C(C)(C)C)cc5)cc3)c3c(-c5ccccc5)nn(-c5ccccc5)c34)cc2)cc1 ; CC(C)(C)c1ccc(N(c2ccc(C(C)(C)C)cc2)c2ccc3c(c2)N(c2ccc(-c4ccccc4)cc2)c2cccc4c2B3c2c(c(-c3ccccc3)nn2-c2ccccc2)N4c2ccc(-c3ccccc3)cc2)cc1 ; c1ccc(-c2ccc(N(c3ccc(-c4ccccc4)cc3)c3ccc4c(c3)N(c3ccc(-c5ccccc5)cc3)c3cccc5c3B4c3c(c(-c4ccccc4)nn3-c3ccccc3)N5c3ccc(-c4ccccc4)cc3)cc2)cc1]

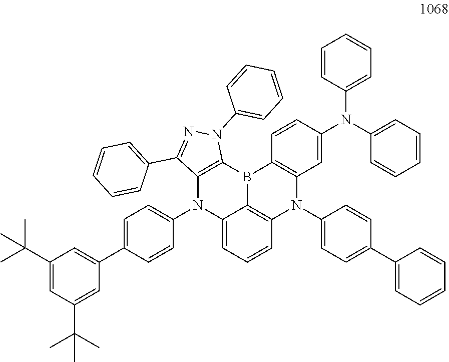 [SMILES: CC(C)(C)c1cc(-c2ccc(N3c4cccc5c4B(c4ccc(N(c6ccccc6)c6ccccc6)cc4N5c4ccc(-c5ccccc5)cc4)c4c3c(-c3ccccc3)nn4-c3ccccc3)cc2)cc(C(C)(C)C)c1]

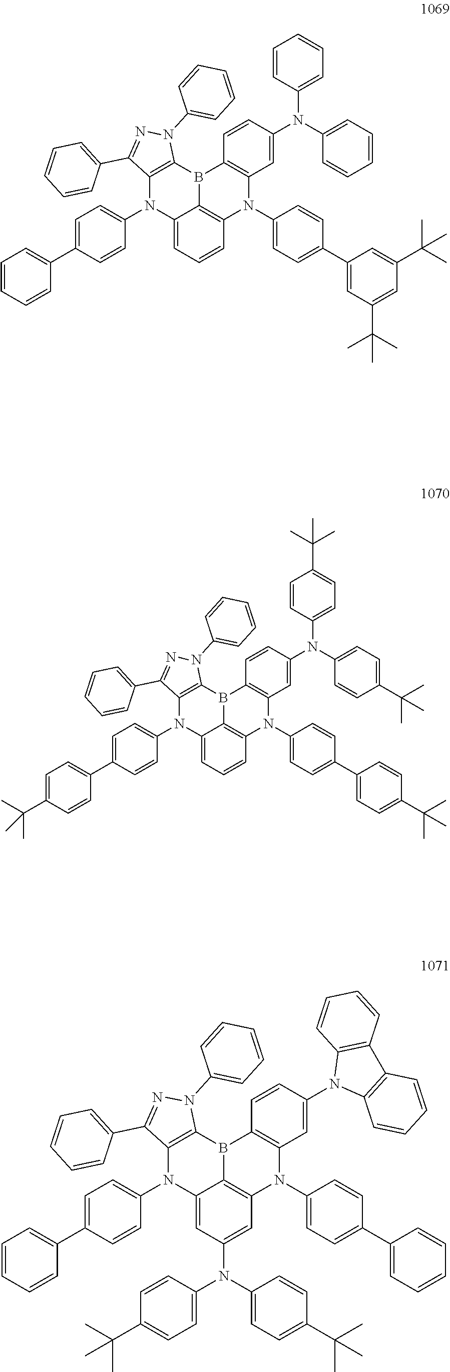

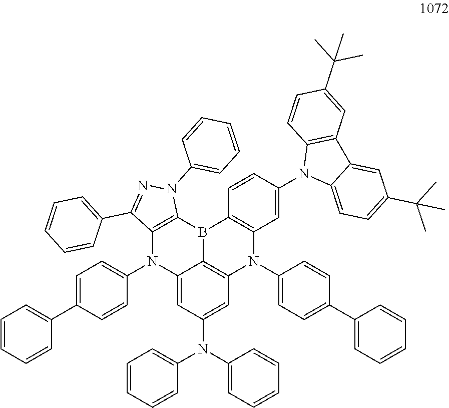 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(c(-c2ccccc2)nn1-c1ccccc1)N3c1ccc(-c2ccccc2)cc1]

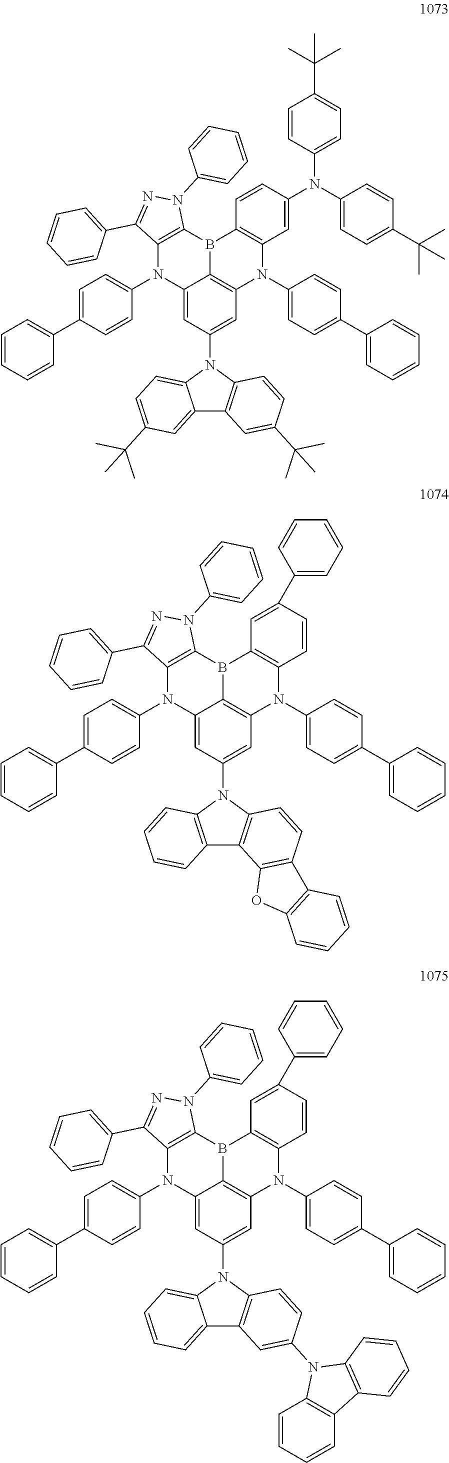

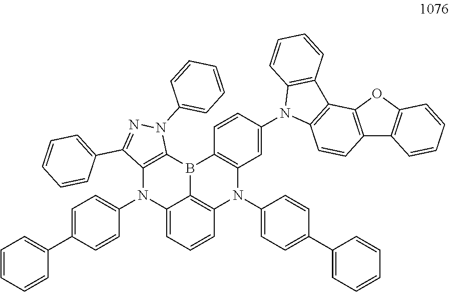 [SMILES: c1ccc(-c2ccc(N3c4cc(-n5c6ccccc6c6c7oc8ccccc8c7ccc65)ccc4B4c5c3cccc5N(c3ccc(-c5ccccc5)cc3)c3c(-c5ccccc5)nn(-c5ccccc5)c34)cc2)cc1]

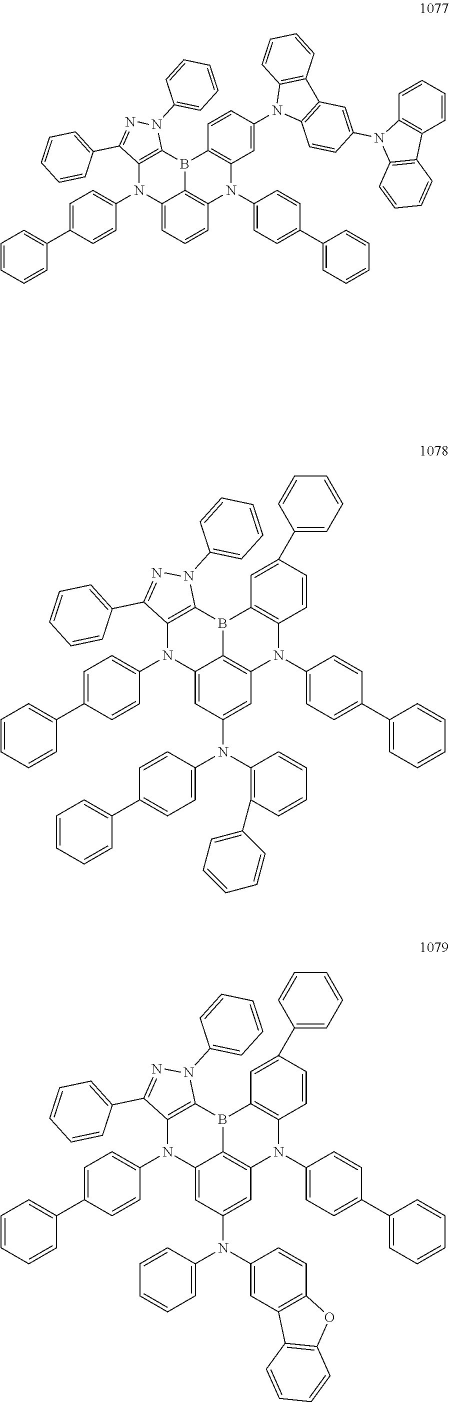 [SMILES: c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3c(-c5ccccc5)nn(-c5ccccc5)c34)cc2)cc1 ; c1ccc(-c2ccc(N(c3cc4c5c(c3)N(c3ccc(-c6ccccc6)cc3)c3c(-c6ccccc6)nn(-c6ccccc6)c3B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3c(-c5ccccc5)nn(-c5ccccc5)c34)cc2)cc1]

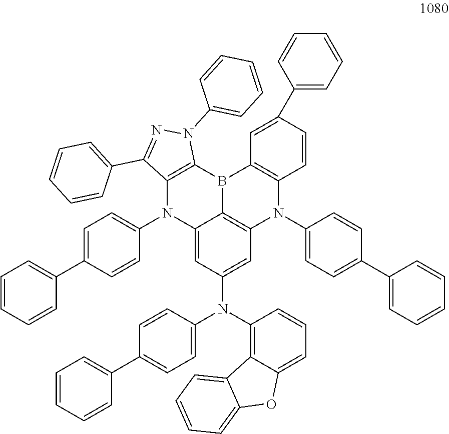 [SMILES: c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3c(-c5ccccc5)nn(-c5ccccc5)c34)cc2)cc1]

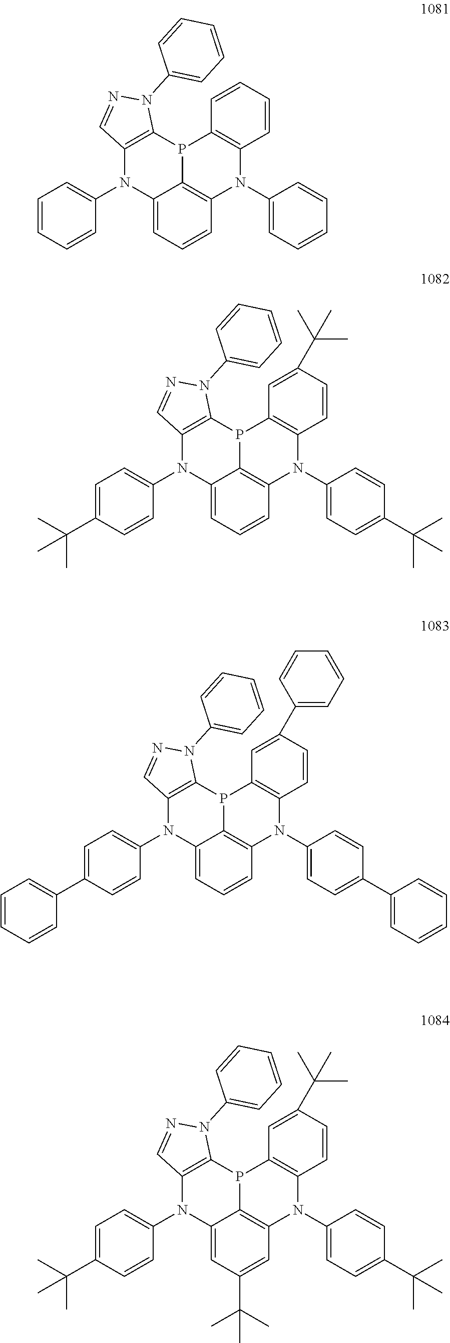

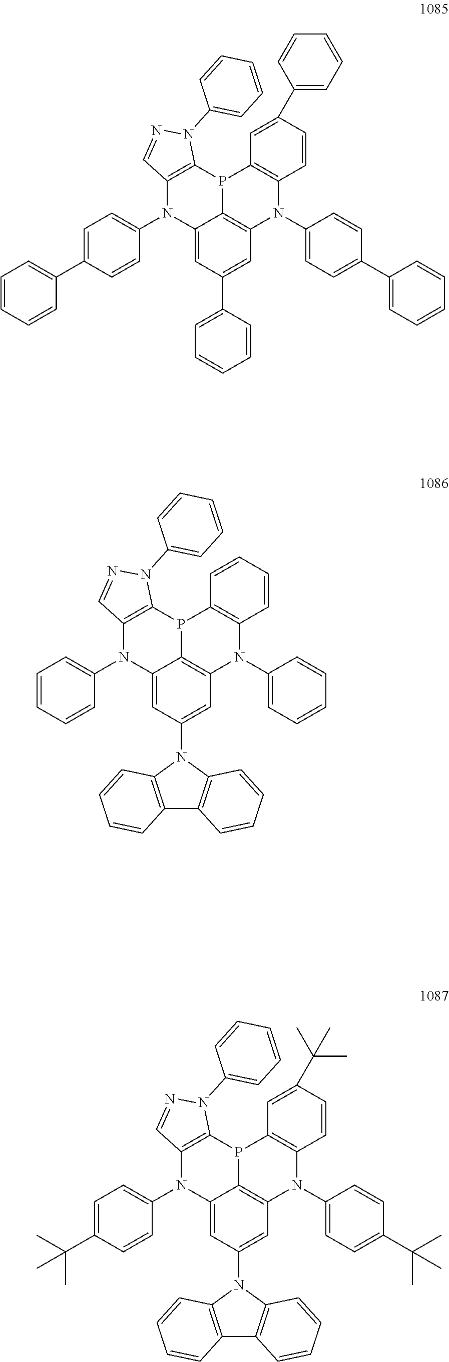 [SMILES: c1ccc(-c2ccc(N3c4ccc(-c5ccccc5)cc4P4c5c3cc(-c3ccccc3)cc5N(c3ccc(-c5ccccc5)cc3)c3cnn(-c5ccccc5)c34)cc2)cc1 ; c1ccc(N2c3ccccc3P3c4c2cc(-n2c5ccccc5c5ccccc52)cc4N(c2ccccc2)c2cnn(-c4ccccc4)c23)cc1 ; CC(C)(C)c1ccc(N2c3ccc(C(C)(C)C)cc3P3c4c2cc(-n2c5ccccc5c5ccccc52)cc4N(c2ccc(C(C)(C)C)cc2)c2cnn(-c4ccccc4)c23)cc1]

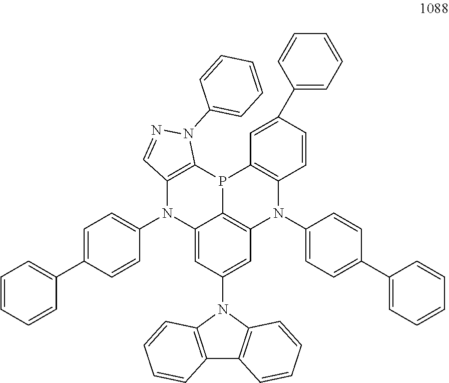 [SMILES: c1ccc(-c2ccc(N3c4ccc(-c5ccccc5)cc4P4c5c3cc(-n3c6ccccc6c6ccccc63)cc5N(c3ccc(-c5ccccc5)cc3)c3cnn(-c5ccccc5)c34)cc2)cc1]

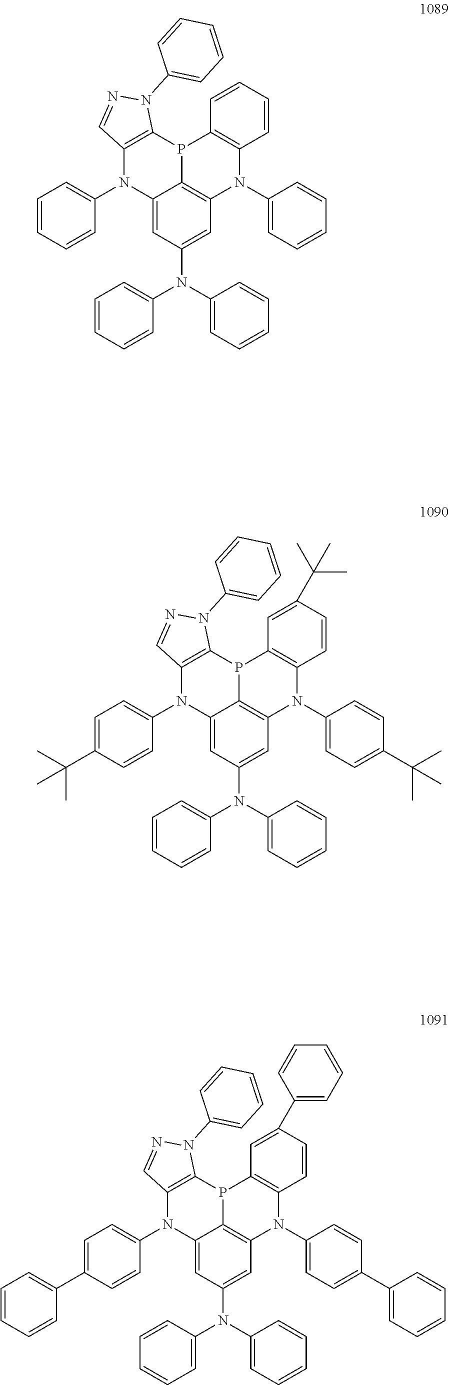 [SMILES: c1ccc(N(c2ccccc2)c2cc3c4c(c2)N(c2ccccc2)c2cnn(-c5ccccc5)c2P4c2ccccc2N3c2ccccc2)cc1 ; CC(C)(C)c1ccc(N2c3ccc(C(C)(C)C)cc3P3c4c2cc(N(c2ccccc2)c2ccccc2)cc4N(c2ccc(C(C)(C)C)cc2)c2cnn(-c4ccccc4)c23)cc1 ; c1ccc(-c2ccc(N3c4ccc(-c5ccccc5)cc4P4c5c3cc(N(c3ccccc3)c3ccccc3)cc5N(c3ccc(-c5ccccc5)cc3)c3cnn(-c5ccccc5)c34)cc2)cc1]

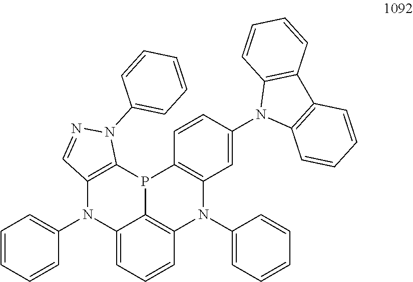 [SMILES: c1ccc(N2c3cc(-n4c5ccccc5c5ccccc54)ccc3P3c4c2cccc4N(c2ccccc2)c2cnn(-c4ccccc4)c23)cc1]

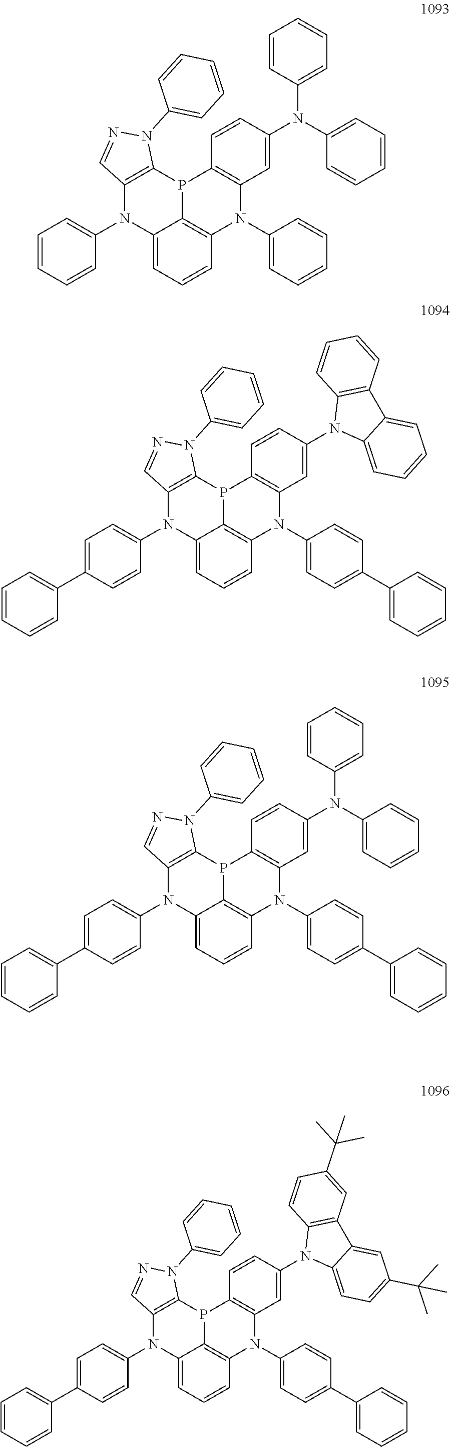 [SMILES: c1ccc(N(c2ccccc2)c2ccc3c(c2)N(c2ccccc2)c2cccc4c2P3c2c(cnn2-c2ccccc2)N4c2ccccc2)cc1 ; c1ccc(-c2ccc(N3c4cc(-n5c6ccccc6c6ccccc65)ccc4P4c5c3cccc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4P4c5c3cccc5N(c3ccc(-c5ccccc5)cc3)c3cnn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1c(cnn1-c1ccccc1)N3c1ccc(-c2ccccc2)cc1]

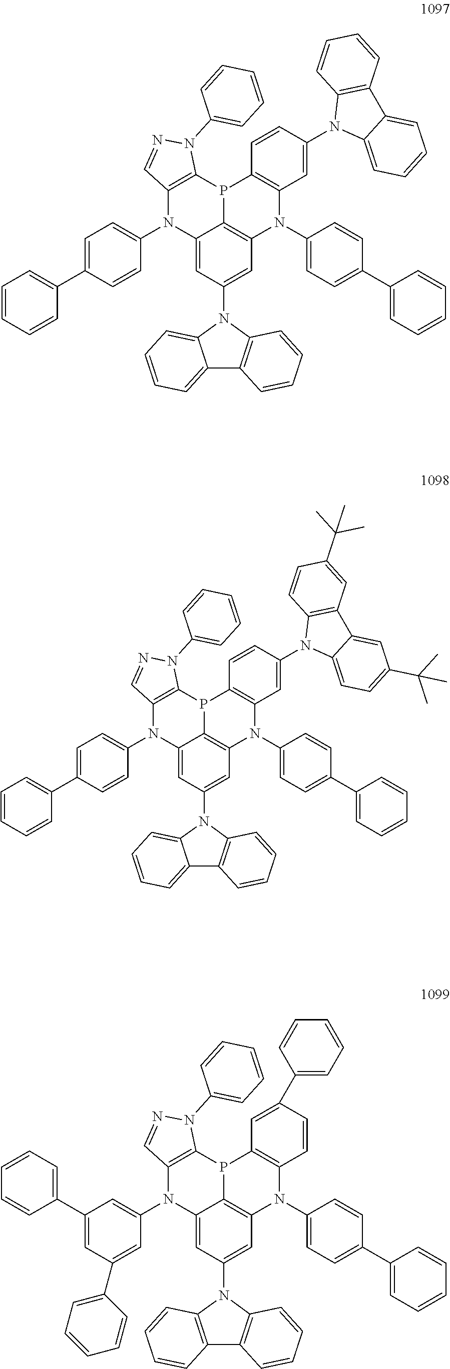 [SMILES: c1ccc(-c2ccc(N3c4cc(-n5c6ccccc6c6ccccc65)ccc4P4c5c3cc(-n3c6ccccc6c6ccccc63)cc5N(c3ccc(-c5ccccc5)cc3)c3cnn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1c(cnn1-c1ccccc1)N3c1ccc(-c2ccccc2)cc1 ; c1ccc(-c2ccc(N3c4ccc(-c5ccccc5)cc4P4c5c3cc(-n3c6ccccc6c6ccccc63)cc5N(c3cc(-c5ccccc5)cc(-c5ccccc5)c3)c3cnn(-c5ccccc5)c34)cc2)cc1]

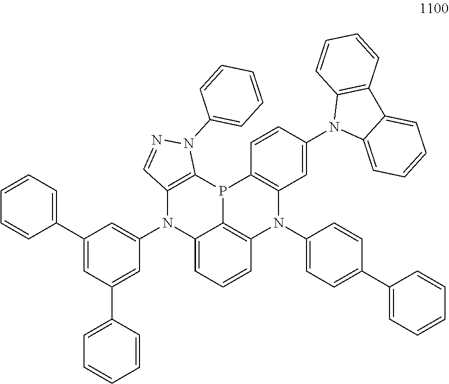 [SMILES: c1ccc(-c2ccc(N3c4cc(-n5c6ccccc6c6ccccc65)ccc4P4c5c3cccc5N(c3cc(-c5ccccc5)cc(-c5ccccc5)c3)c3cnn(-c5ccccc5)c34)cc2)cc1]

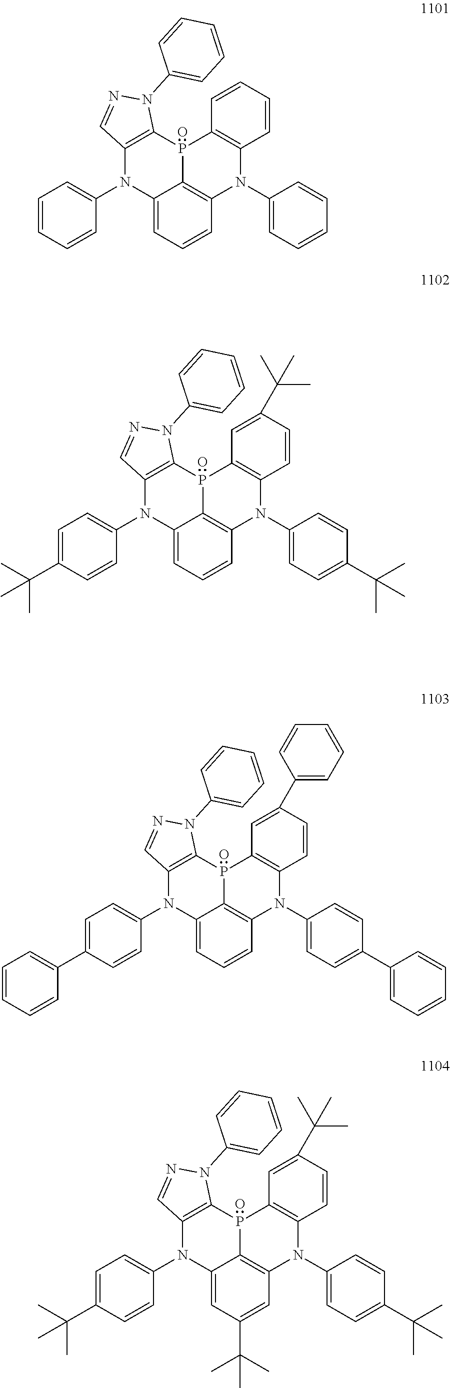

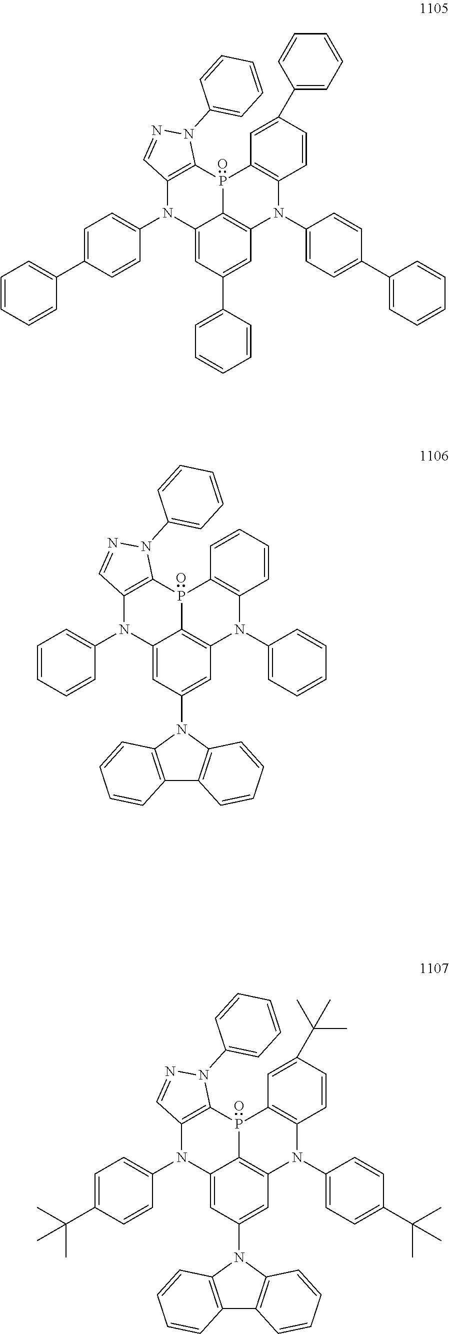

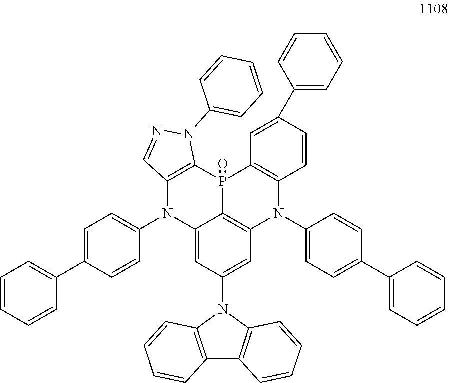

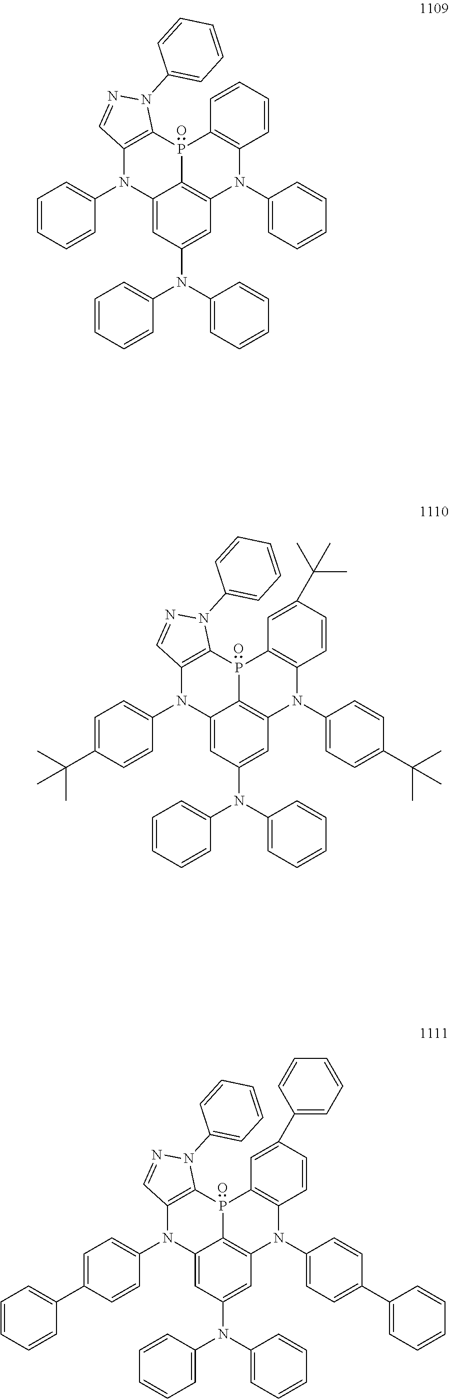

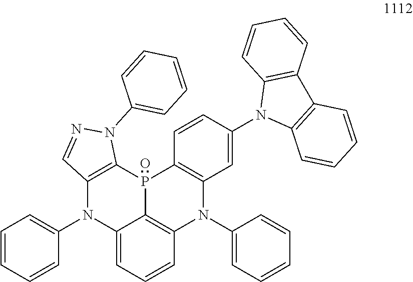

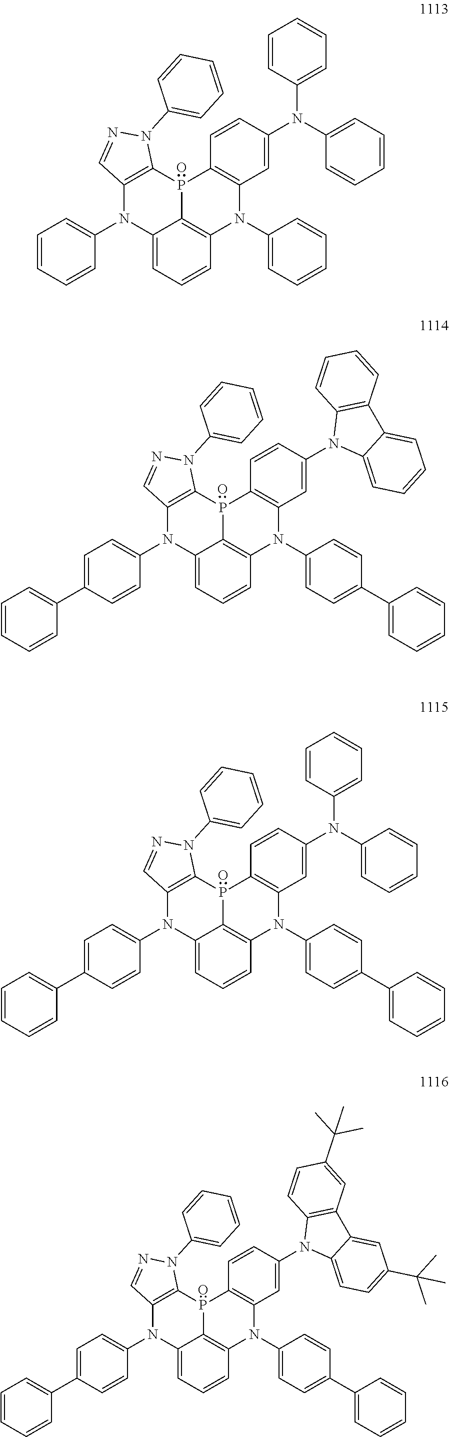

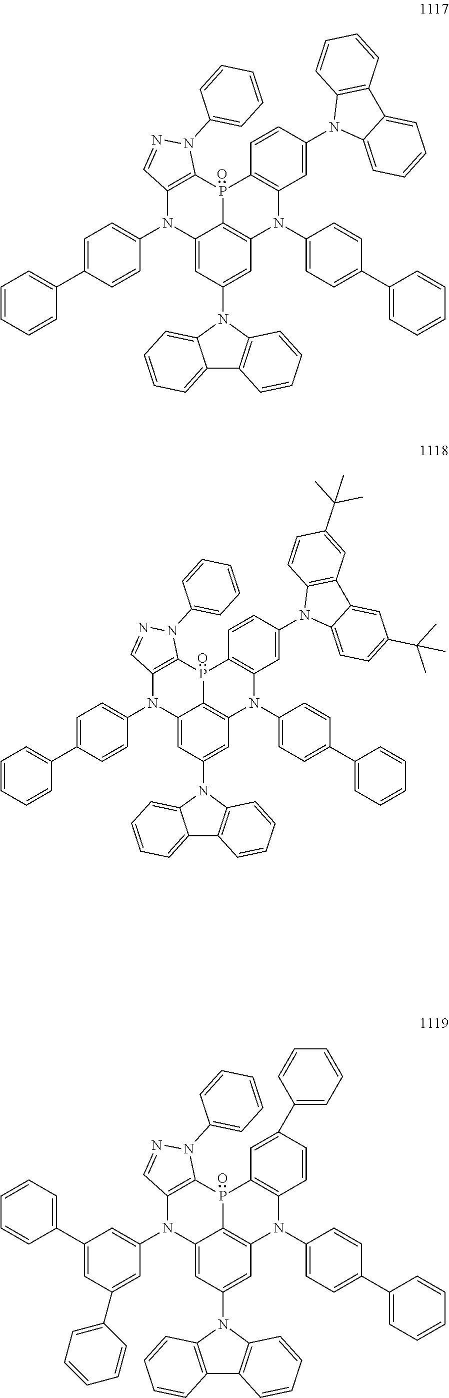

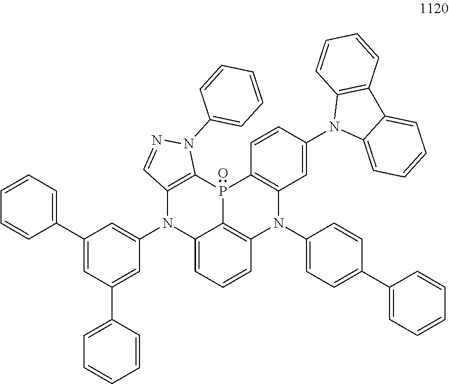

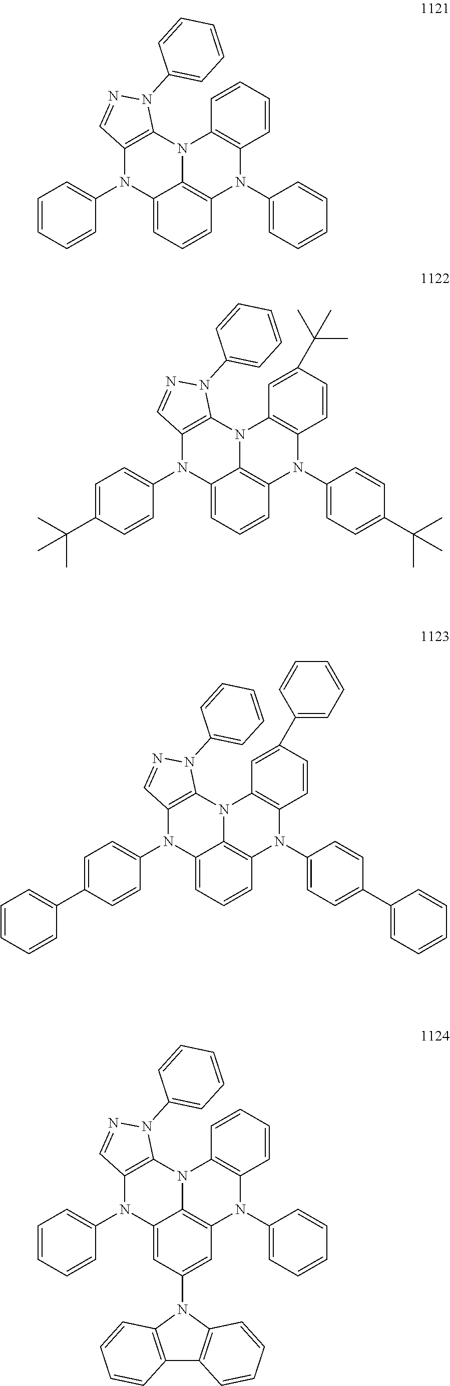 [SMILES: c1ccc(N2c3ccccc3N3c4c2cccc4N(c2ccccc2)c2cnn(-c4ccccc4)c23)cc1 ; CC(C)(C)c1ccc(N2c3ccc(C(C)(C)C)cc3N3c4c2cccc4N(c2ccc(C(C)(C)C)cc2)c2cnn(-c4ccccc4)c23)cc1 ; c1ccc(-c2ccc(N3c4ccc(-c5ccccc5)cc4N4c5c3cccc5N(c3ccc(-c5ccccc5)cc3)c3cnn(-c5ccccc5)c34)cc2)cc1 ; c1ccc(N2c3ccccc3N3c4c2cc(-n2c5ccccc5c5ccccc52)cc4N(c2ccccc2)c2cnn(-c4ccccc4)c23)cc1]

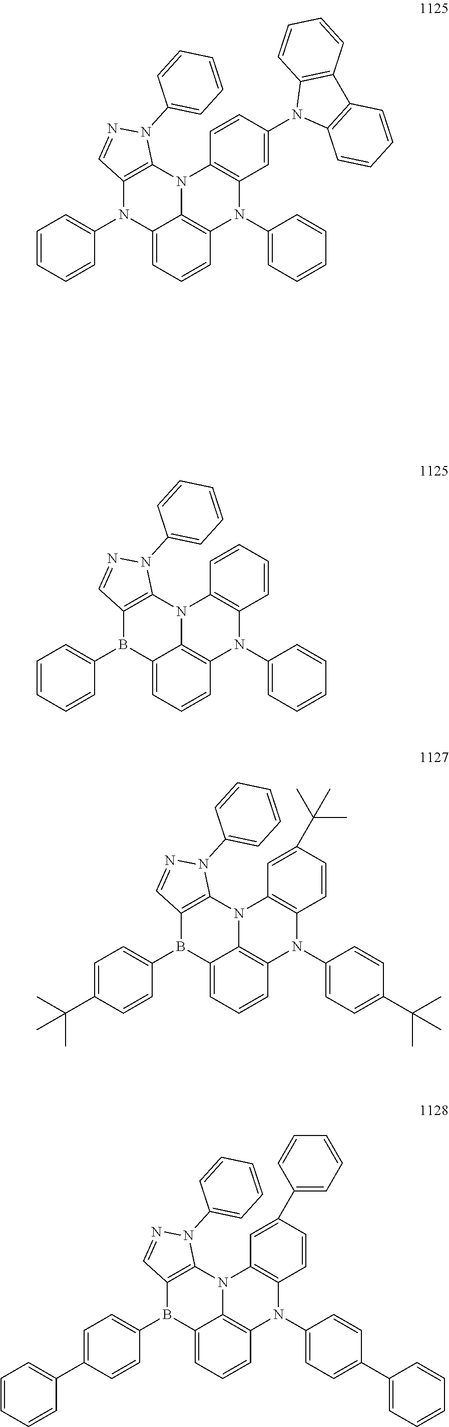 [SMILES: c1ccc(N2c3cc(-n4c5ccccc5c5ccccc54)ccc3N3c4c2cccc4N(c2ccccc2)c2cnn(-c4ccccc4)c23)cc1 ; c1ccc(B2c3cccc4c3N(c3ccccc3N4c3ccccc3)c3c2cnn3-c2ccccc2)cc1 ; CC(C)(C)c1ccc(B2c3cccc4c3N(c3cc(C(C)(C)C)ccc3N4c3ccc(C(C)(C)C)cc3)c3c2cnn3-c2ccccc2)cc1 ; c1ccc(-c2ccc(B3c4cccc5c4N(c4cc(-c6ccccc6)ccc4N5c4ccc(-c5ccccc5)cc4)c4c3cnn4-c3ccccc3)cc2)cc1]

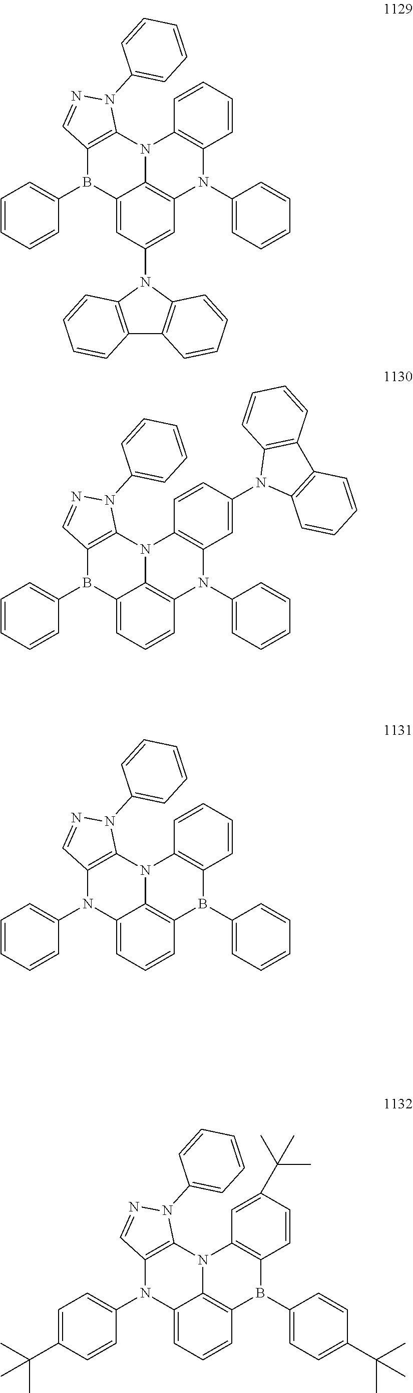 [SMILES: c1ccc(B2c3cc(-n4c5ccccc5c5ccccc54)cc4c3N(c3ccccc3N4c3ccccc3)c3c2cnn3-c2ccccc2)cc1 ; c1ccc(B2c3cccc4c3N(c3ccc(-n5c6ccccc6c6ccccc65)cc3N4c3ccccc3)c3c2cnn3-c2ccccc2)cc1 ; c1ccc(B2c3ccccc3N3c4c2cccc4N(c2ccccc2)c2cnn(-c4ccccc4)c23)cc1 ; CC(C)(C)c1ccc(B2c3ccc(C(C)(C)C)cc3N3c4c2cccc4N(c2ccc(C(C)(C)C)cc2)c2cnn(-c4ccccc4)c23)cc1]

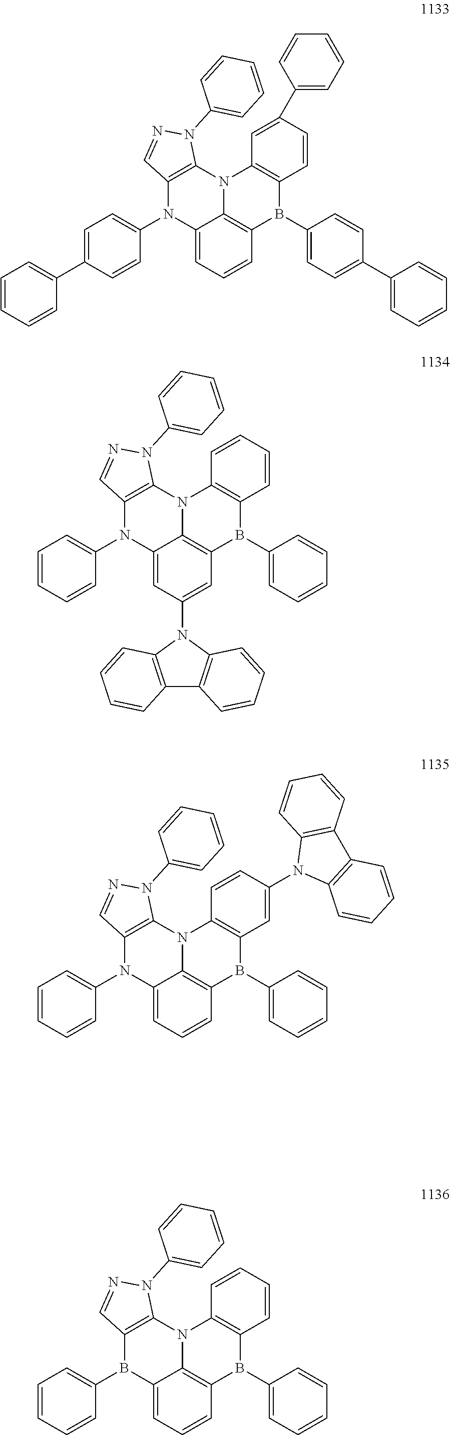 [SMILES: c1ccc(-c2ccc(B3c4ccc(-c5ccccc5)cc4N4c5c3cccc5N(c3ccc(-c5ccccc5)cc3)c3cnn(-c5ccccc5)c34)cc2)cc1 ; c1ccc(B2c3ccccc3N3c4c2cc(-n2c5ccccc5c5ccccc52)cc4N(c2ccccc2)c2cnn(-c4ccccc4)c23)cc1 ; c1ccc(B2c3cc(-n4c5ccccc5c5ccccc54)ccc3N3c4c2cccc4N(c2ccccc2)c2cnn(-c4ccccc4)c23)cc1 ; c1ccc(B2c3ccccc3N3c4c2cccc4B(c2ccccc2)c2cnn(-c4ccccc4)c23)cc1]

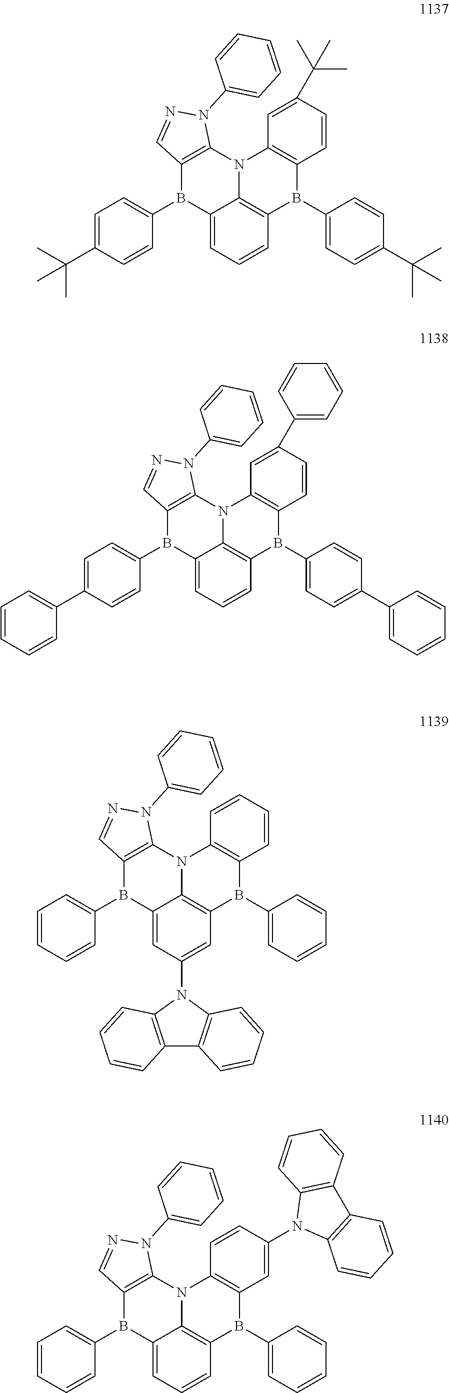 [SMILES: CC(C)(C)c1ccc(B2c3ccc(C(C)(C)C)cc3N3c4c2cccc4B(c2ccc(C(C)(C)C)cc2)c2cnn(-c4ccccc4)c23)cc1 ; c1ccc(-c2ccc(B3c4ccc(-c5ccccc5)cc4N4c5c3cccc5B(c3ccc(-c5ccccc5)cc3)c3cnn(-c5ccccc5)c34)cc2)cc1 ; c1ccc(B2c3ccccc3N3c4c2cc(-n2c5ccccc5c5ccccc52)cc4B(c2ccccc2)c2cnn(-c4ccccc4)c23)cc1 ; c1ccc(B2c3cc(-n4c5ccccc5c5ccccc54)ccc3N3c4c2cccc4B(c2ccccc2)c2cnn(-c4ccccc4)c23)cc1]

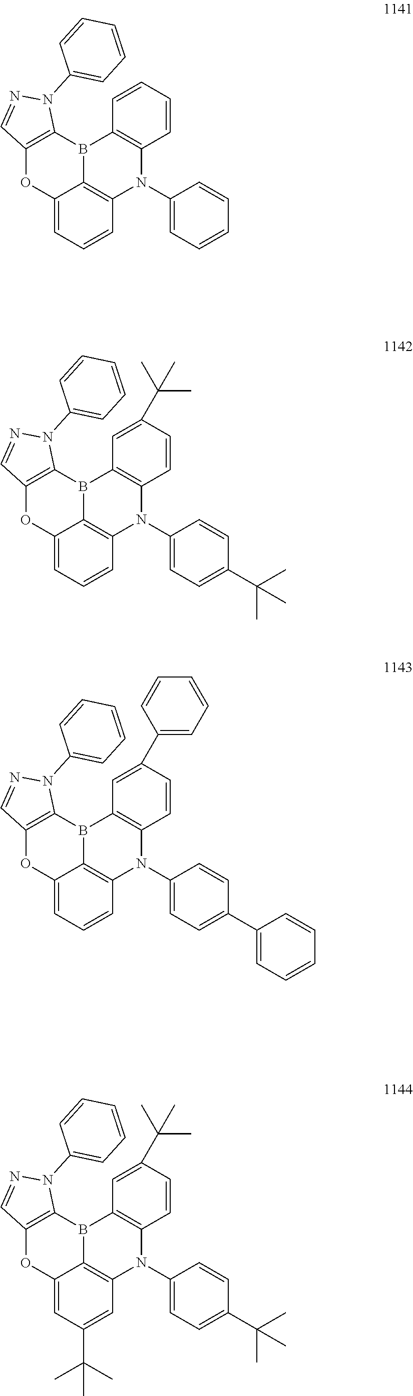 [SMILES: c1ccc(N2c3ccccc3B3c4c(cccc42)Oc2cnn(-c4ccccc4)c23)cc1 ; CC(C)(C)c1ccc(N2c3ccc(C(C)(C)C)cc3B3c4c(cccc42)Oc2cnn(-c4ccccc4)c23)cc1 ; c1ccc(-c2ccc(N3c4ccc(-c5ccccc5)cc4B4c5c(cccc53)Oc3cnn(-c5ccccc5)c34)cc2)cc1 ; CC(C)(C)c1ccc(N2c3ccc(C(C)(C)C)cc3B3c4c(cc(C(C)(C)C)cc42)Oc2cnn(-c4ccccc4)c23)cc1]

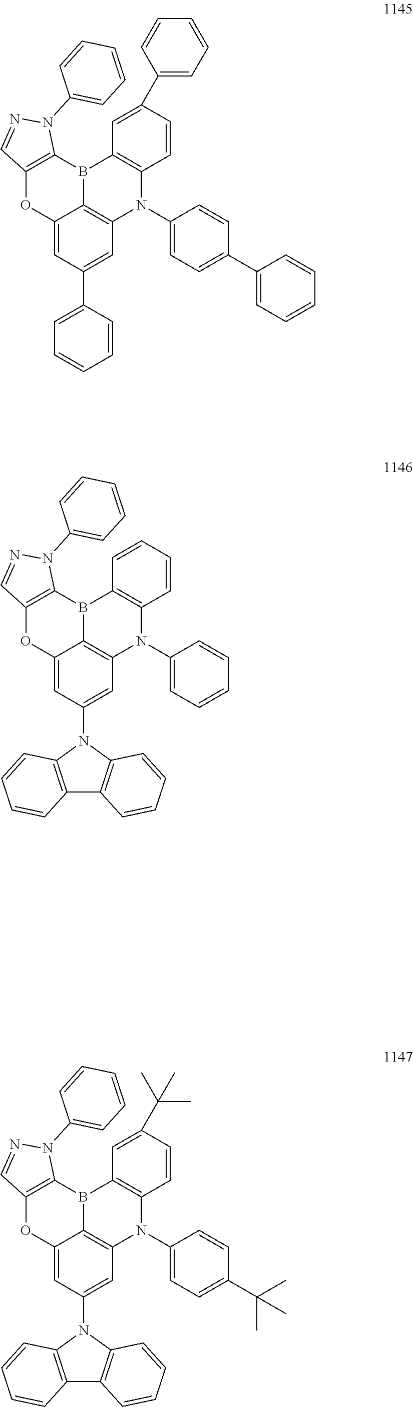 [SMILES: c1ccc(-c2ccc(N3c4ccc(-c5ccccc5)cc4B4c5c(cc(-c6ccccc6)cc53)Oc3cnn(-c5ccccc5)c34)cc2)cc1 ; c1ccc(N2c3ccccc3B3c4c(cc(-n5c6ccccc6c6ccccc65)cc42)Oc2cnn(-c4ccccc4)c23)cc1 ; CC(C)(C)c1ccc(N2c3ccc(C(C)(C)C)cc3B3c4c(cc(-n5c6ccccc6c6ccccc65)cc42)Oc2cnn(-c4ccccc4)c23)cc1]

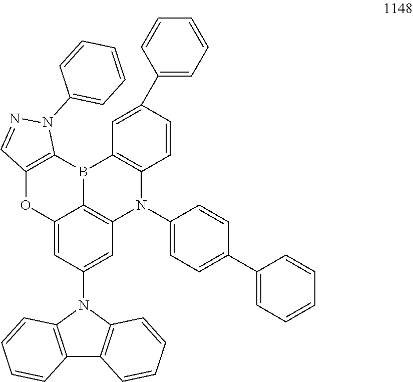 [SMILES: c1ccc(-c2ccc(N3c4ccc(-c5ccccc5)cc4B4c5c(cc(-n6c7ccccc7c7ccccc76)cc53)Oc3cnn(-c5ccccc5)c34)cc2)cc1]

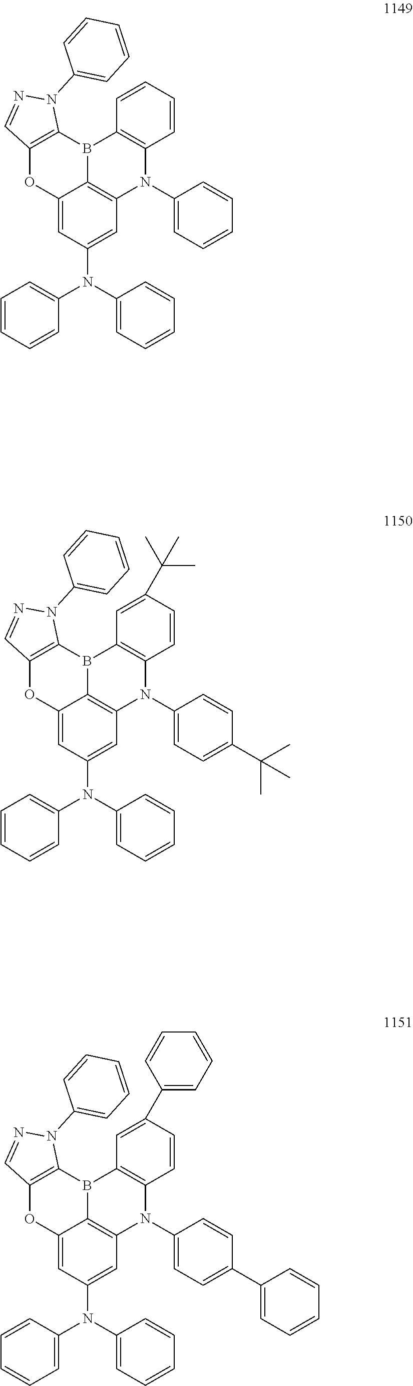 [SMILES: c1ccc(N(c2ccccc2)c2cc3c4c(c2)N(c2ccccc2)c2ccccc2B4c2c(cnn2-c2ccccc2)O3)cc1 ; CC(C)(C)c1ccc(N2c3ccc(C(C)(C)C)cc3B3c4c(cc(N(c5ccccc5)c5ccccc5)cc42)Oc2cnn(-c4ccccc4)c23)cc1 ; c1ccc(-c2ccc(N3c4ccc(-c5ccccc5)cc4B4c5c(cc(N(c6ccccc6)c6ccccc6)cc53)Oc3cnn(-c5ccccc5)c34)cc2)cc1]

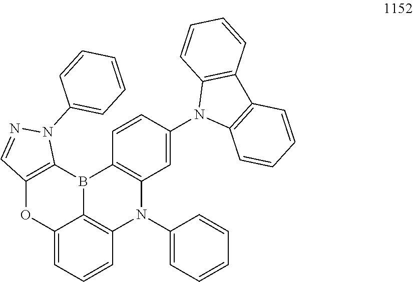 [SMILES: c1ccc(N2c3cc(-n4c5ccccc5c5ccccc54)ccc3B3c4c(cccc42)Oc2cnn(-c4ccccc4)c23)cc1]

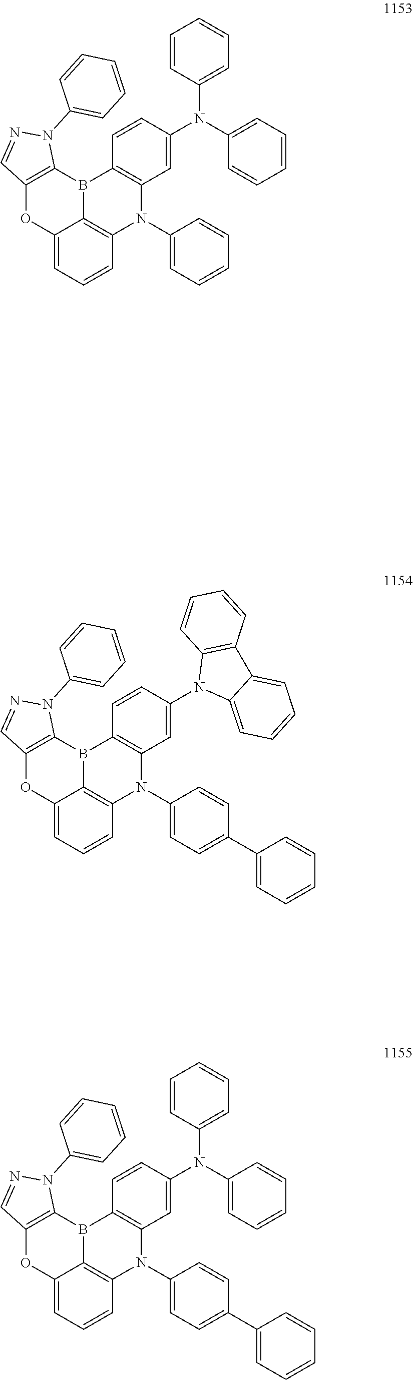 [SMILES: c1ccc(N(c2ccccc2)c2ccc3c(c2)N(c2ccccc2)c2cccc4c2B3c2c(cnn2-c2ccccc2)O4)cc1 ; c1ccc(-c2ccc(N3c4cc(-n5c6ccccc6c6ccccc65)ccc4B4c5c(cccc53)Oc3cnn(-c5ccccc5)c34)cc2)cc1 ; c1ccc(-c2ccc(N3c4cc(N(c5ccccc5)c5ccccc5)ccc4B4c5c(cccc53)Oc3cnn(-c5ccccc5)c34)cc2)cc1]

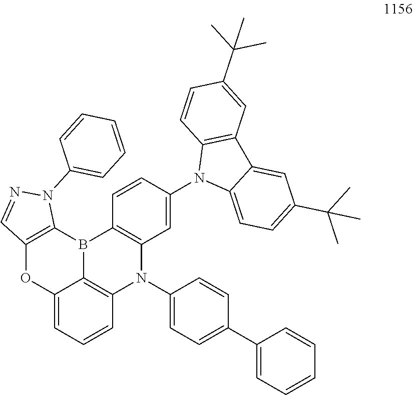 [SMILES: CC(C)(C)c1ccc2c(c1)c1cc(C(C)(C)C)ccc1n2-c1ccc2c(c1)N(c1ccc(-c3ccccc3)cc1)c1cccc3c1B2c1c(cnn1-c1ccccc1)O3]

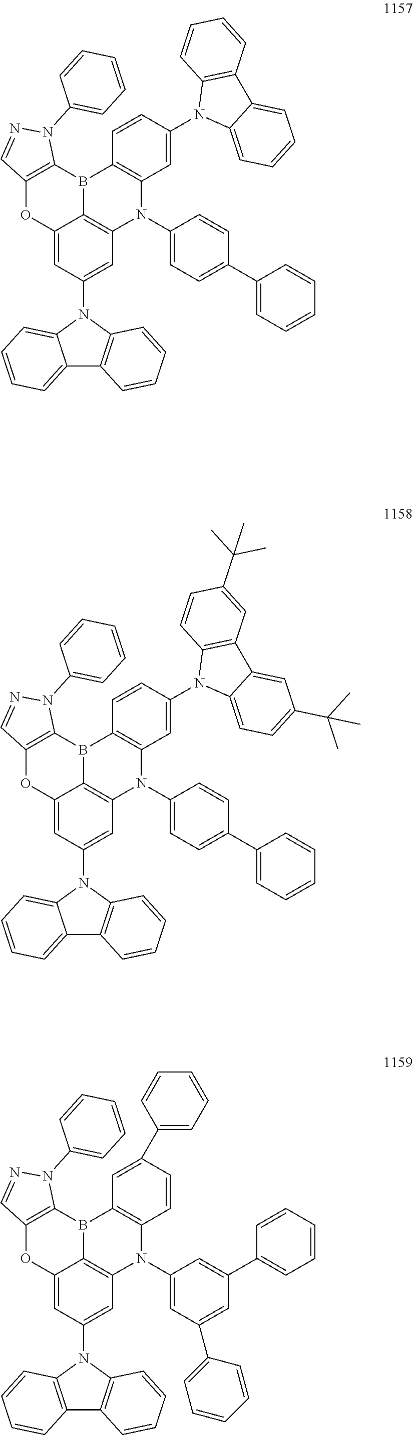 [SMILES: c1ccc(-c2ccc(N3c4cc(-n5c6ccccc6c6ccccc65)ccc4B4c5c(cc(-n6c7ccccc7c7ccccc76)cc53)Oc3cnn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(cnn1-c1ccccc1)O3 ; c1ccc(-c2cc(-c3ccccc3)cc(N3c4ccc(-c5ccccc5)cc4B4c5c(cc(-n6c7ccccc7c7ccccc76)cc53)Oc3cnn(-c5ccccc5)c34)c2)cc1]

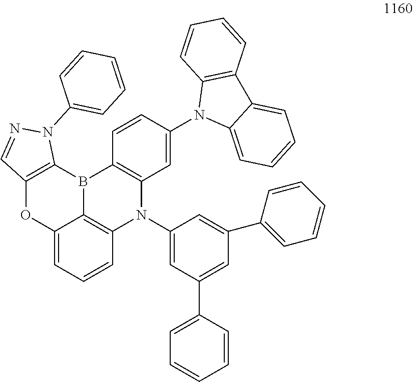 [SMILES: c1ccc(-c2cc(-c3ccccc3)cc(N3c4cc(-n5c6ccccc6c6ccccc65)ccc4B4c5c(cccc53)Oc3cnn(-c5ccccc5)c34)c2)cc1]

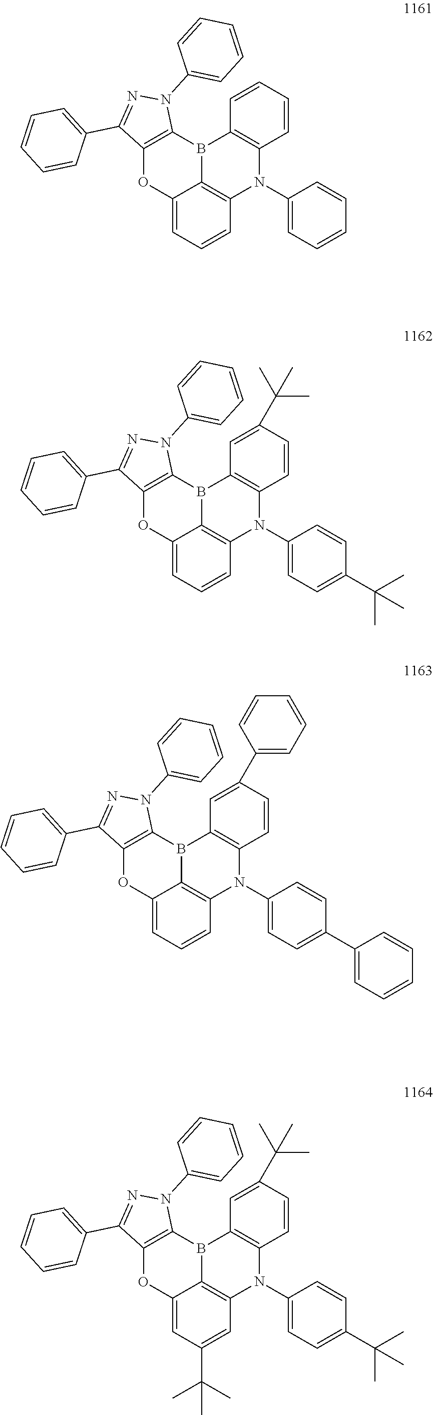 [SMILES: c1ccc(-c2nn(-c3ccccc3)c3c2Oc2cccc4c2B3c2ccccc2N4c2ccccc2)cc1 ; CC(C)(C)c1ccc(N2c3ccc(C(C)(C)C)cc3B3c4c(cccc42)Oc2c(-c4ccccc4)nn(-c4ccccc4)c23)cc1 ; c1ccc(-c2ccc(N3c4ccc(-c5ccccc5)cc4B4c5c(cccc53)Oc3c(-c5ccccc5)nn(-c5ccccc5)c34)cc2)cc1 ; CC(C)(C)c1ccc(N2c3ccc(C(C)(C)C)cc3B3c4c(cc(C(C)(C)C)cc42)Oc2c(-c4ccccc4)nn(-c4ccccc4)c23)cc1]

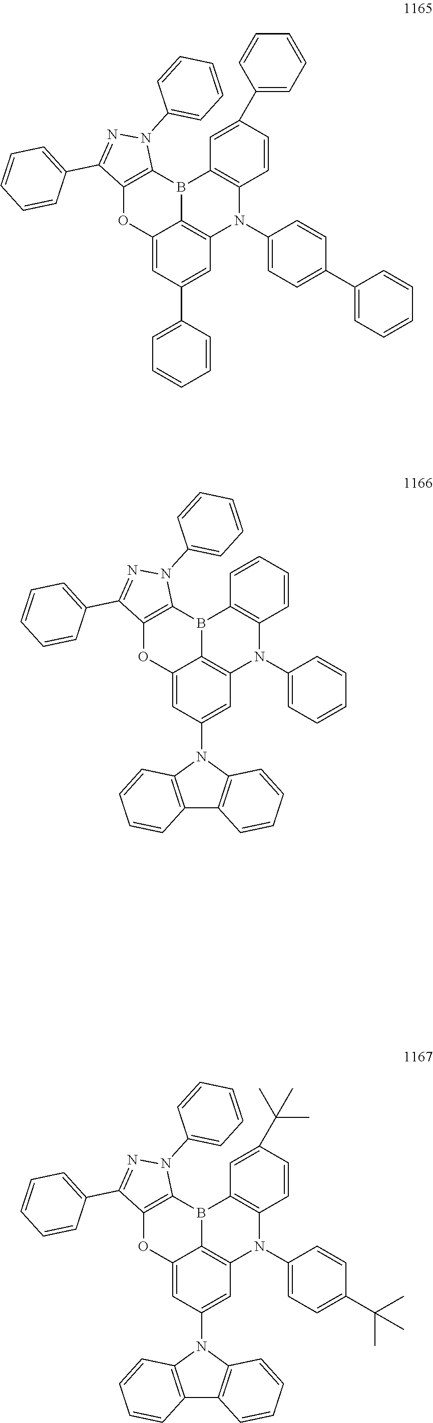 [SMILES: c1ccc(-c2ccc(N3c4ccc(-c5ccccc5)cc4B4c5c(cc(-c6ccccc6)cc53)Oc3c(-c5ccccc5)nn(-c5ccccc5)c34)cc2)cc1 ; c1ccc(-c2nn(-c3ccccc3)c3c2Oc2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4c(cc(-n5c6ccccc6c6ccccc65)cc42)Oc2c(-c4ccccc4)nn(-c4ccccc4)c23)cc1]

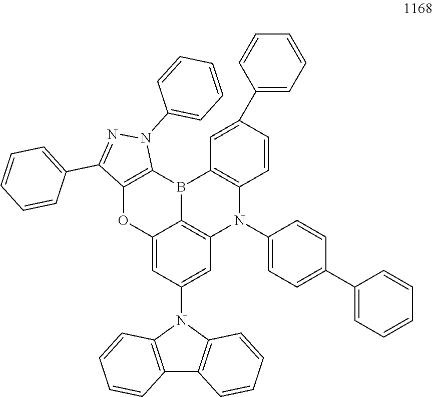 [SMILES: c1ccc(-c2ccc(N3c4ccc(-c5ccccc5)cc4B4c5c(cc(-n6c7ccccc7c7ccccc76)cc53)Oc3c(-c5ccccc5)nn(-c5ccccc5)c34)cc2)cc1]

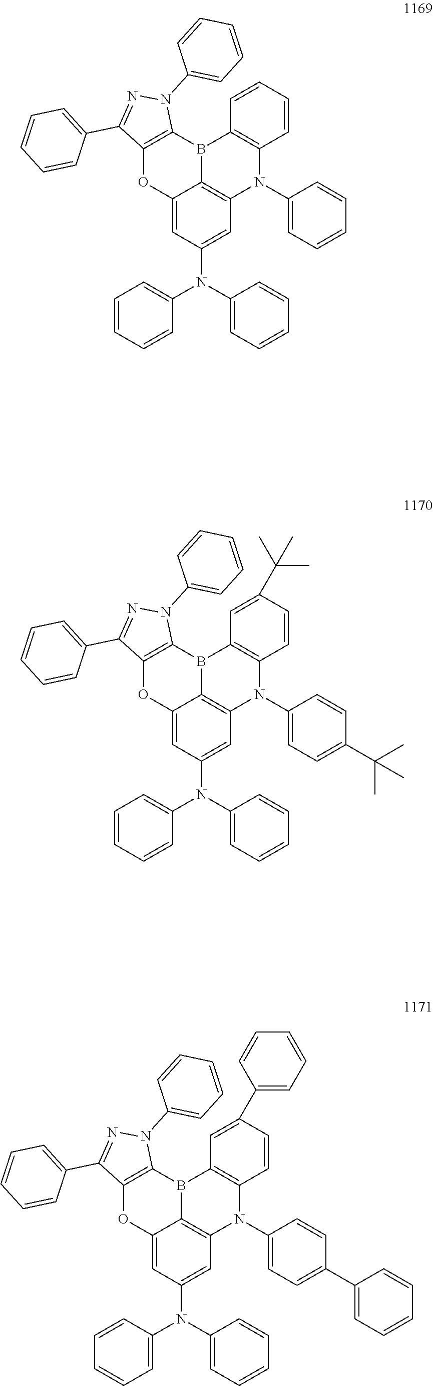 [SMILES: c1ccc(-c2nn(-c3ccccc3)c3c2Oc2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4c(cc(N(c5ccccc5)c5ccccc5)cc42)Oc2c(-c4ccccc4)nn(-c4ccccc4)c23)cc1 ; c1ccc(-c2ccc(N3c4ccc(-c5ccccc5)cc4B4c5c(cc(N(c6ccccc6)c6ccccc6)cc53)Oc3c(-c5ccccc5)nn(-c5ccccc5)c34)cc2)cc1]

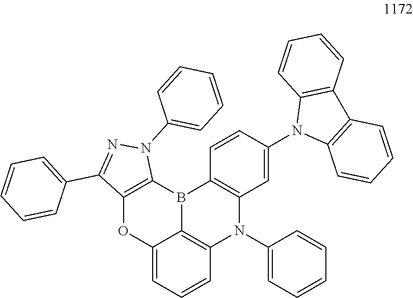 [SMILES: c1ccc(-c2nn(-c3ccccc3)c3c2Oc2cccc4c2B3c2ccc(-n3c5ccccc5c5ccccc53)cc2N4c2ccccc2)cc1]

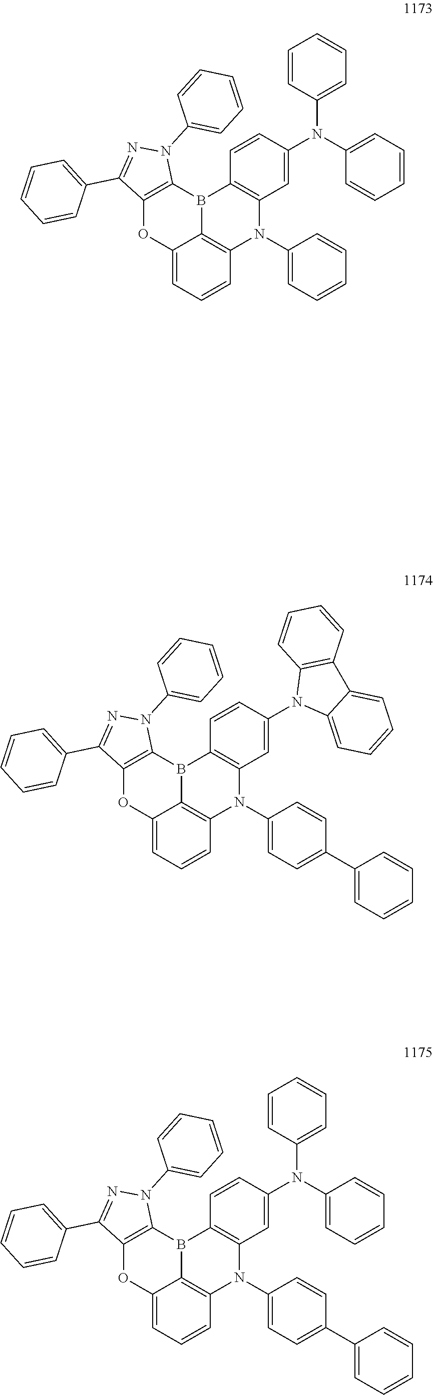 [SMILES: c1ccc(-c2nn(-c3ccccc3)c3c2Oc2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5c(cccc53)Oc3c(-c5ccccc5)nn(-c5ccccc5)c34)cc2)cc1 ; c1ccc(-c2ccc(N3c4cc(N(c5ccccc5)c5ccccc5)ccc4B4c5c(cccc53)Oc3c(-c5ccccc5)nn(-c5ccccc5)c34)cc2)cc1]

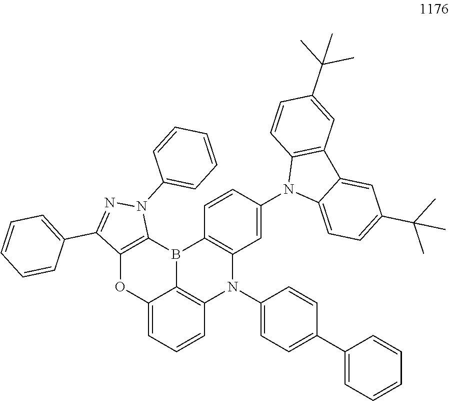 [SMILES: CC(C)(C)c1ccc2c(c1)c1cc(C(C)(C)C)ccc1n2-c1ccc2c(c1)N(c1ccc(-c3ccccc3)cc1)c1cccc3c1B2c1c(c(-c2ccccc2)nn1-c1ccccc1)O3]

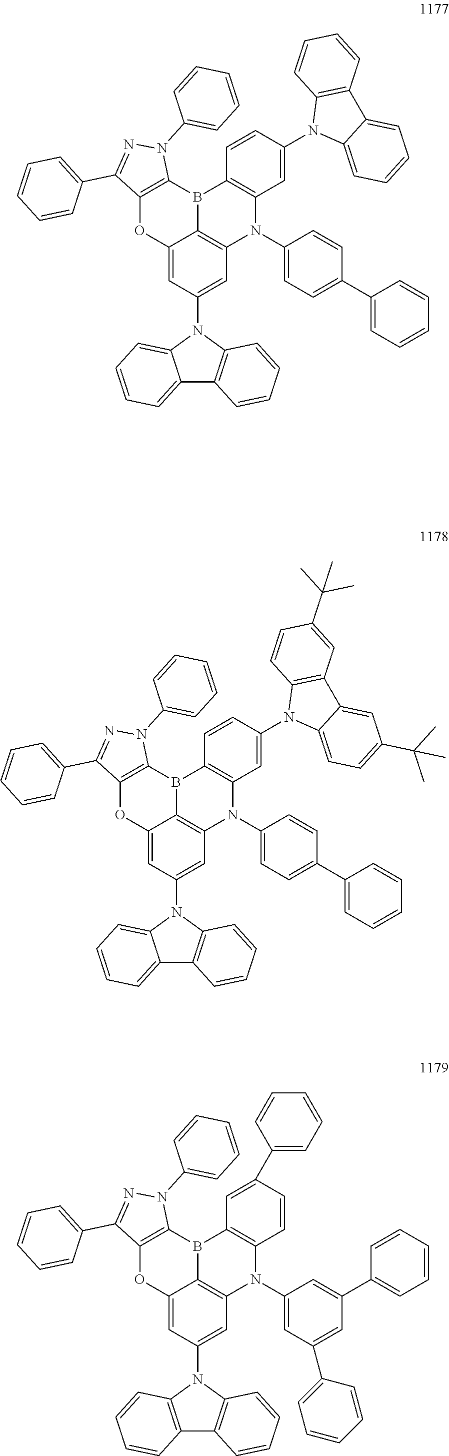 [SMILES: c1ccc(-c2ccc(N3c4cc(-n5c6ccccc6c6ccccc65)ccc4B4c5c(cc(-n6c7ccccc7c7ccccc76)cc53)Oc3c(-c5ccccc5)nn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(c(-c2ccccc2)nn1-c1ccccc1)O3 ; c1ccc(-c2cc(-c3ccccc3)cc(N3c4ccc(-c5ccccc5)cc4B4c5c(cc(-n6c7ccccc7c7ccccc76)cc53)Oc3c(-c5ccccc5)nn(-c5ccccc5)c34)c2)cc1]

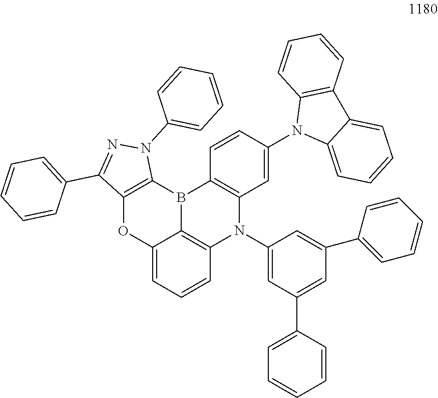 [SMILES: c1ccc(-c2cc(-c3ccccc3)cc(N3c4cc(-n5c6ccccc6c6ccccc65)ccc4B4c5c(cccc53)Oc3c(-c5ccccc5)nn(-c5ccccc5)c34)c2)cc1]

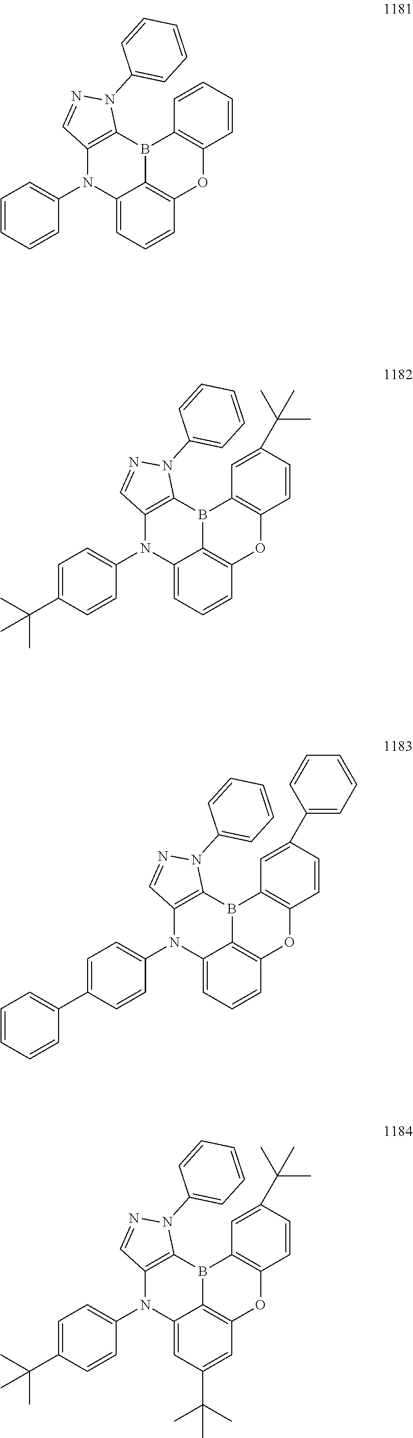

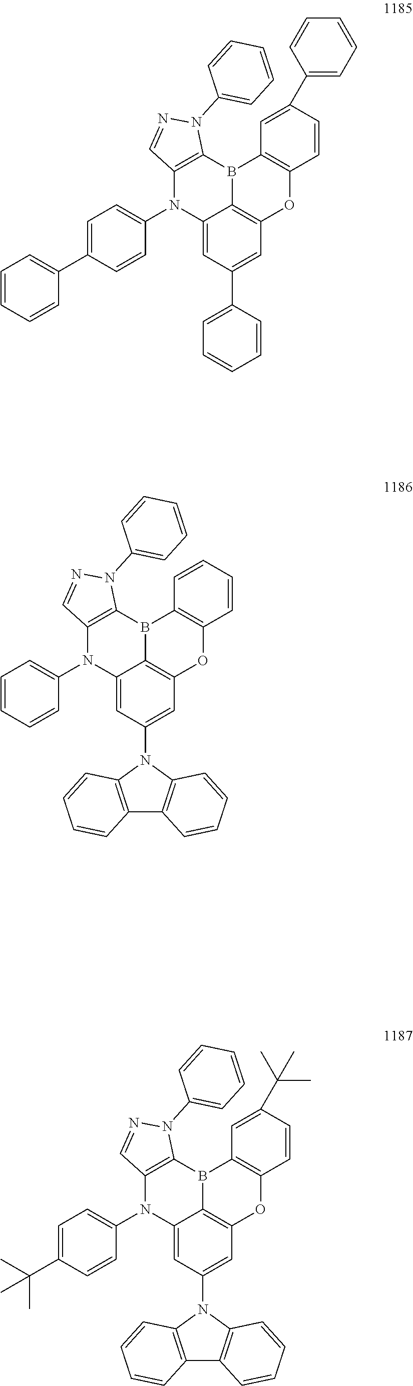 [SMILES: c1ccc(-c2ccc(N3c4cc(-c5ccccc5)cc5c4B(c4cc(-c6ccccc6)ccc4O5)c4c3cnn4-c3ccccc3)cc2)cc1 ; c1ccc(N2c3cc(-n4c5ccccc5c5ccccc54)cc4c3B(c3ccccc3O4)c3c2cnn3-c2ccccc2)cc1 ; CC(C)(C)c1ccc(N2c3cc(-n4c5ccccc5c5ccccc54)cc4c3B(c3cc(C(C)(C)C)ccc3O4)c3c2cnn3-c2ccccc2)cc1]

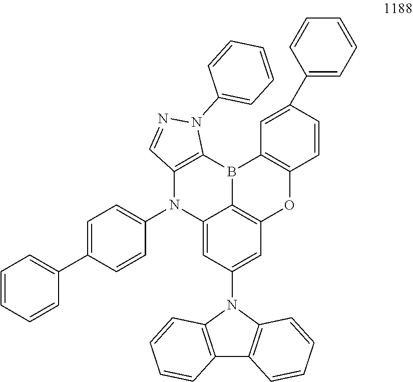 [SMILES: c1ccc(-c2ccc(N3c4cc(-n5c6ccccc6c6ccccc65)cc5c4B(c4cc(-c6ccccc6)ccc4O5)c4c3cnn4-c3ccccc3)cc2)cc1]

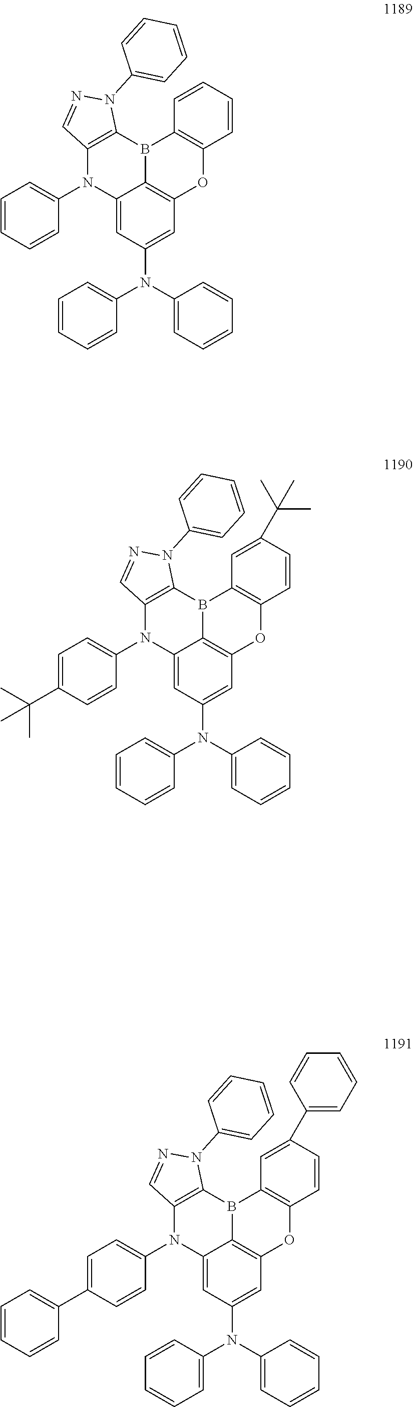 [SMILES: c1ccc(N(c2ccccc2)c2cc3c4c(c2)N(c2ccccc2)c2cnn(-c5ccccc5)c2B4c2ccccc2O3)cc1 ; CC(C)(C)c1ccc(N2c3cc(N(c4ccccc4)c4ccccc4)cc4c3B(c3cc(C(C)(C)C)ccc3O4)c3c2cnn3-c2ccccc2)cc1 ; c1ccc(-c2ccc(N3c4cc(N(c5ccccc5)c5ccccc5)cc5c4B(c4cc(-c6ccccc6)ccc4O5)c4c3cnn4-c3ccccc3)cc2)cc1]

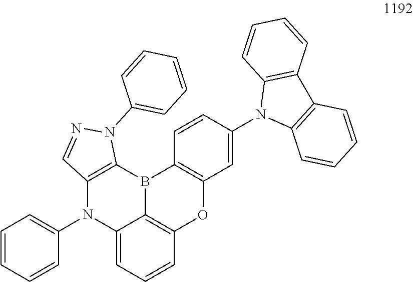 [SMILES: c1ccc(N2c3cccc4c3B(c3ccc(-n5c6ccccc6c6ccccc65)cc3O4)c3c2cnn3-c2ccccc2)cc1]

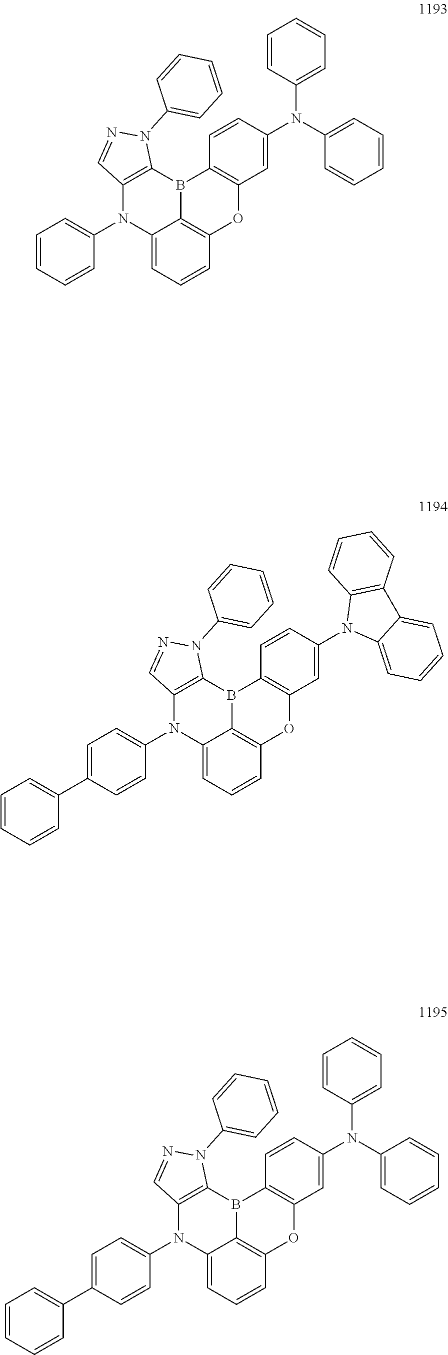 [SMILES: c1ccc(N(c2ccccc2)c2ccc3c(c2)Oc2cccc4c2B3c2c(cnn2-c2ccccc2)N4c2ccccc2)cc1 ; c1ccc(-c2ccc(N3c4cccc5c4B(c4ccc(-n6c7ccccc7c7ccccc76)cc4O5)c4c3cnn4-c3ccccc3)cc2)cc1 ; c1ccc(-c2ccc(N3c4cccc5c4B(c4ccc(N(c6ccccc6)c6ccccc6)cc4O5)c4c3cnn4-c3ccccc3)cc2)cc1]

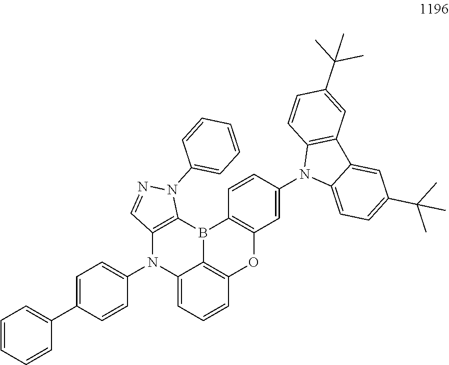 [SMILES: CC(C)(C)c1ccc2c(c1)c1cc(C(C)(C)C)ccc1n2-c1ccc2c(c1)Oc1cccc3c1B2c1c(cnn1-c1ccccc1)N3c1ccc(-c2ccccc2)cc1]

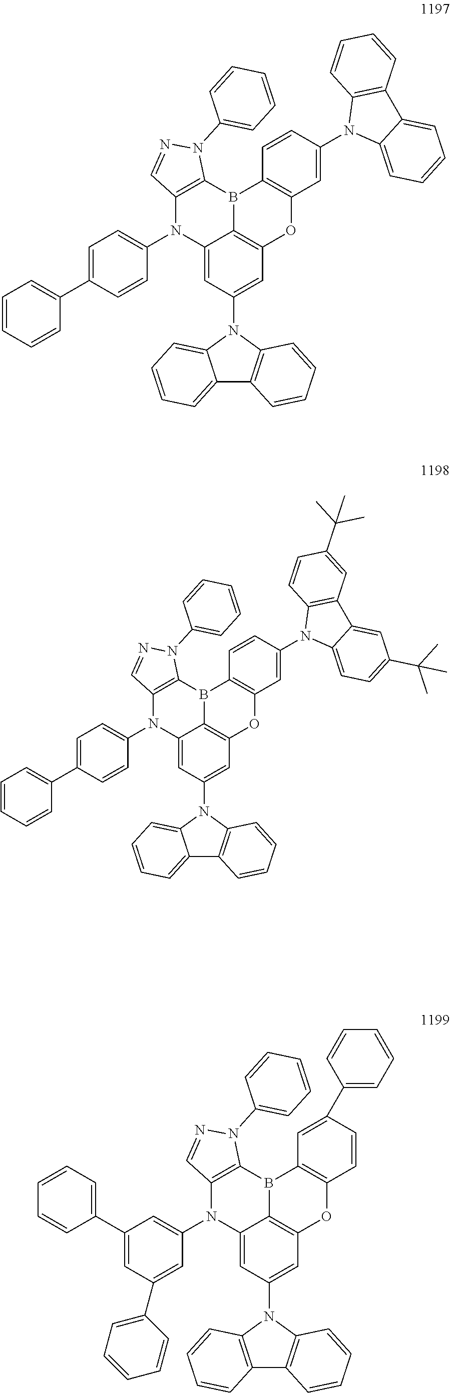 [SMILES: c1ccc(-c2ccc(N3c4cc(-n5c6ccccc6c6ccccc65)cc5c4B(c4ccc(-n6c7ccccc7c7ccccc76)cc4O5)c4c3cnn4-c3ccccc3)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1c(cnn1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3cnn4-c3ccccc3)c2)cc1]

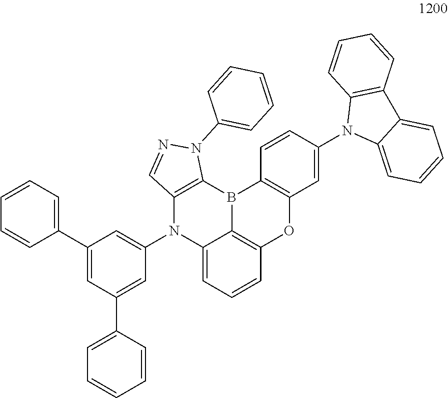 [SMILES: c1ccc(-c2cc(-c3ccccc3)cc(N3c4cccc5c4B(c4ccc(-n6c7ccccc7c7ccccc76)cc4O5)c4c3cnn4-c3ccccc3)c2)cc1]

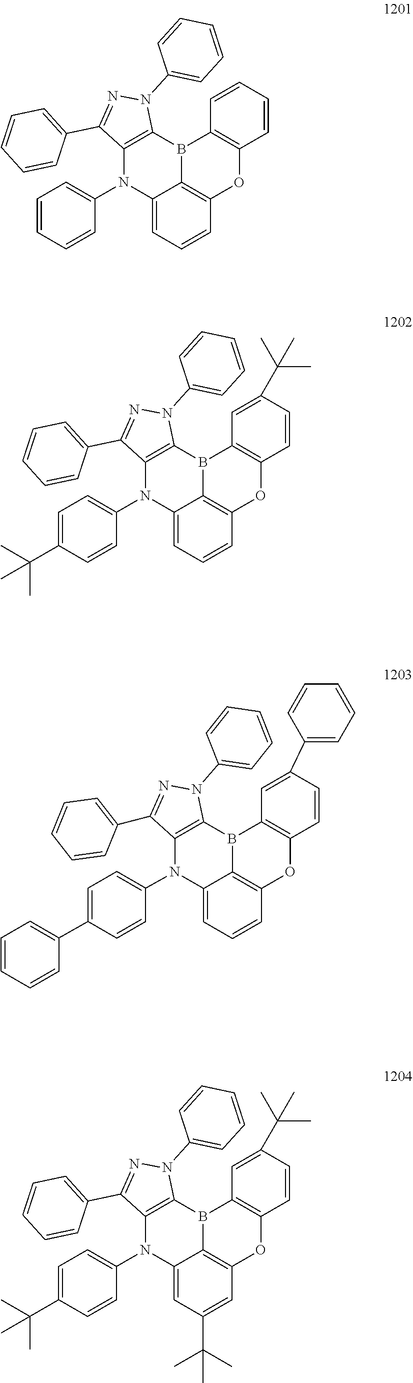

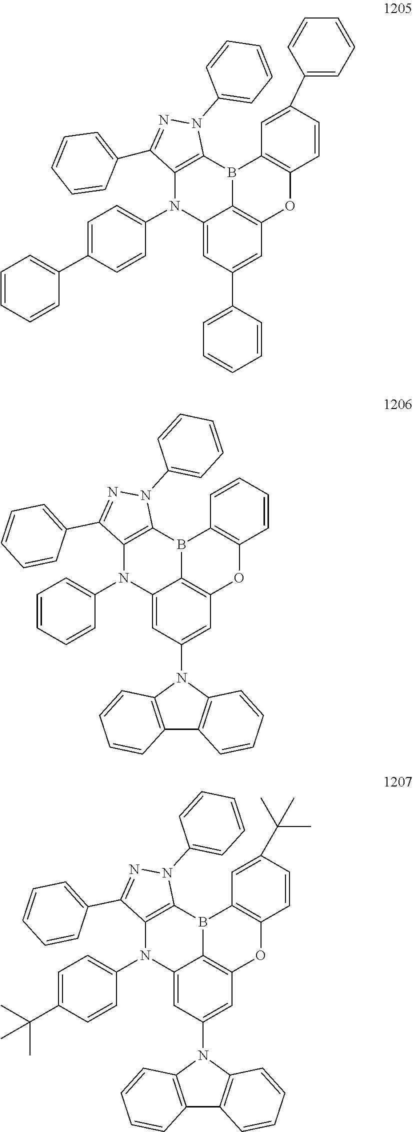

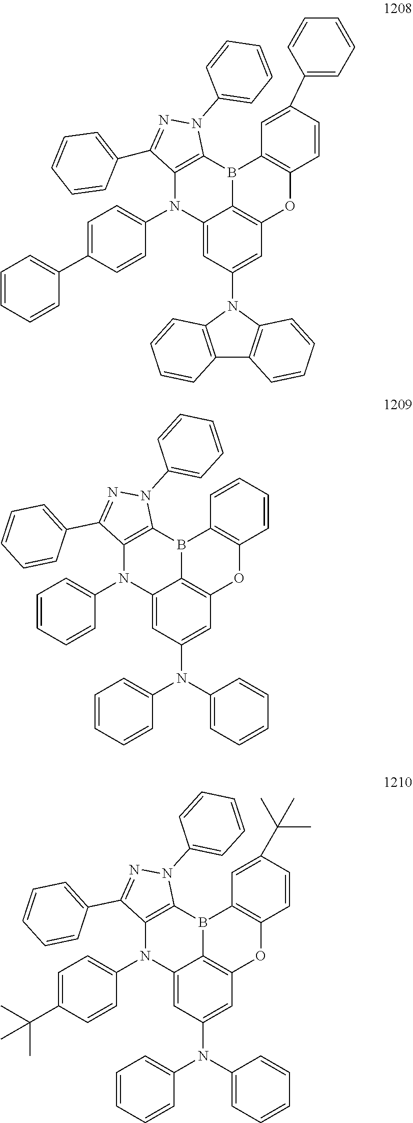

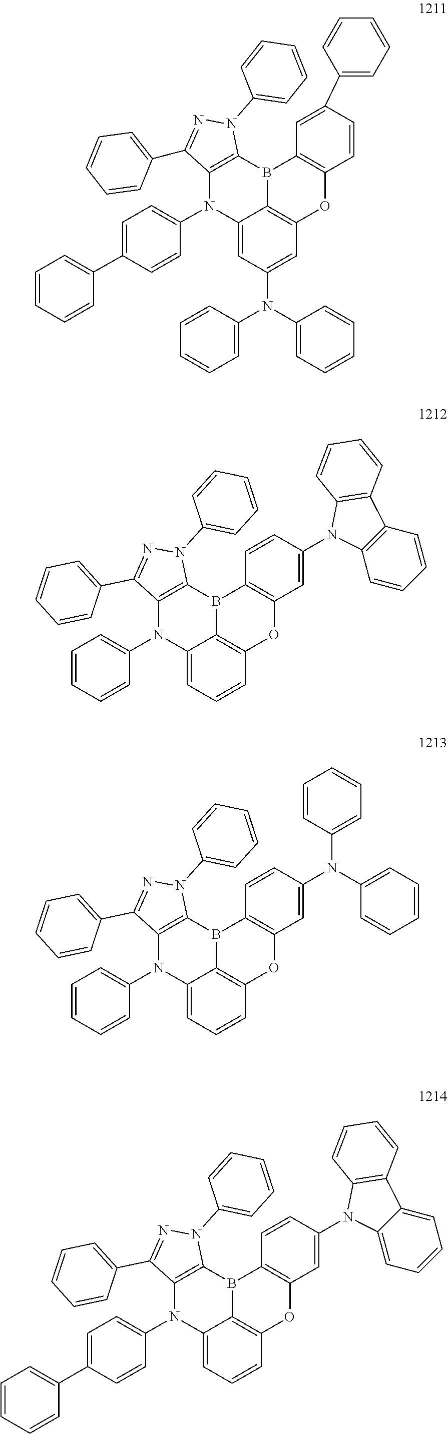 [SMILES: c1ccc(-c2ccc(N3c4cc(N(c5ccccc5)c5ccccc5)cc5c4B(c4cc(-c6ccccc6)ccc4O5)c4c3c(-c3ccccc3)nn4-c3ccccc3)cc2)cc1 ; c1ccc(-c2nn(-c3ccccc3)c3c2N(c2ccccc2)c2cccc4c2B3c2ccc(-n3c5ccccc5c5ccccc53)cc2O4)cc1 ; c1ccc(-c2nn(-c3ccccc3)c3c2N(c2ccccc2)c2cccc4c2B3c2ccc(N(c3ccccc3)c3ccccc3)cc2O4)cc1 ; c1ccc(-c2ccc(N3c4cccc5c4B(c4ccc(-n6c7ccccc7c7ccccc76)cc4O5)c4c3c(-c3ccccc3)nn4-c3ccccc3)cc2)cc1]

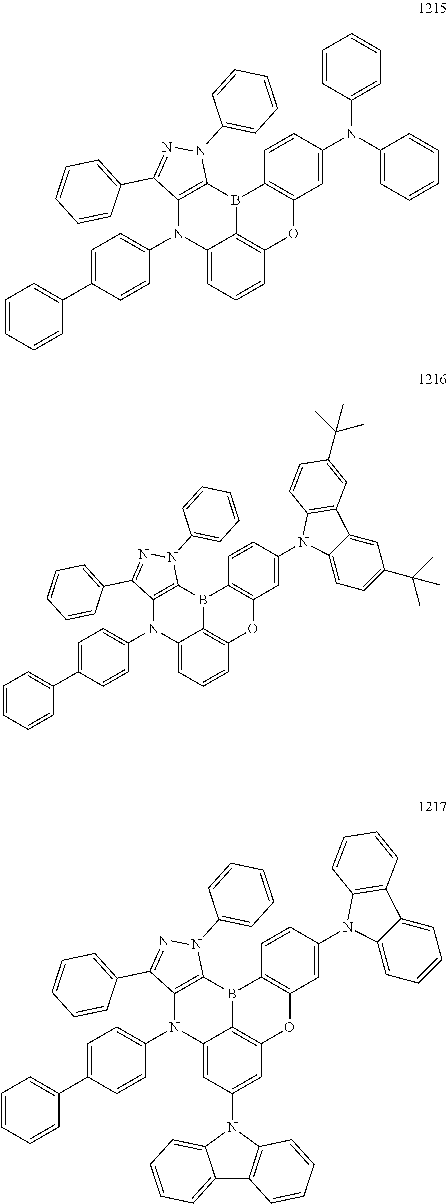 [SMILES: c1ccc(-c2ccc(N3c4cccc5c4B(c4ccc(N(c6ccccc6)c6ccccc6)cc4O5)c4c3c(-c3ccccc3)nn4-c3ccccc3)cc2)cc1 ; CC(C)(C)c1ccc2c(c1)c1cc(C(C)(C)C)ccc1n2-c1ccc2c(c1)Oc1cccc3c1B2c1c(c(-c2ccccc2)nn1-c1ccccc1)N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4c3c(-c3ccccc3)nn4-c3ccccc3)cc2)cc1]

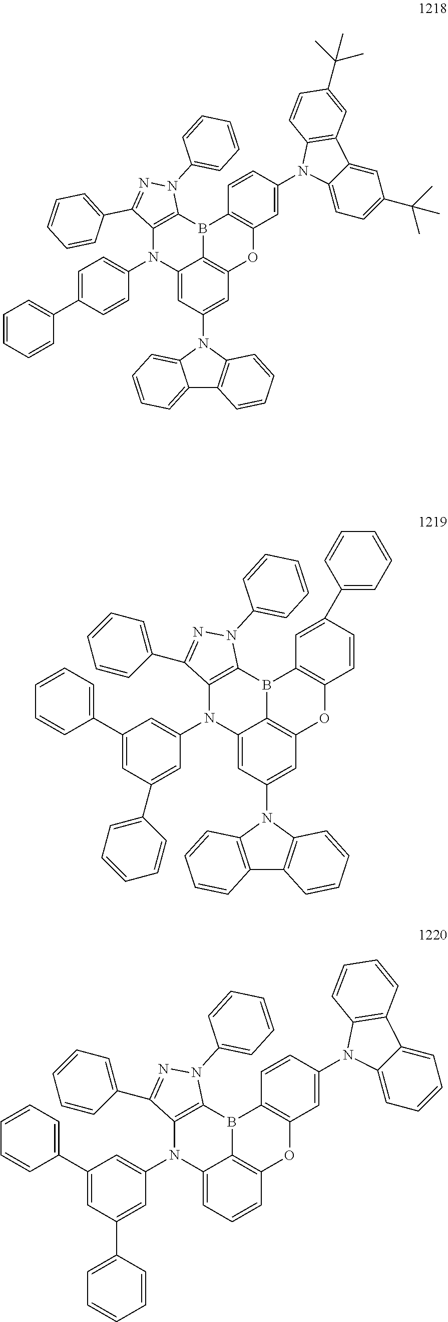 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(c(-c2ccccc2)nn1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3c(-c3ccccc3)nn4-c3ccccc3)c2)cc1 ; c1ccc(-c2cc(-c3ccccc3)cc(N3c4cccc5c4B(c4ccc(-n6c7ccccc7c7ccccc76)cc4O5)c4c3c(-c3ccccc3)nn4-c3ccccc3)c2)cc1]

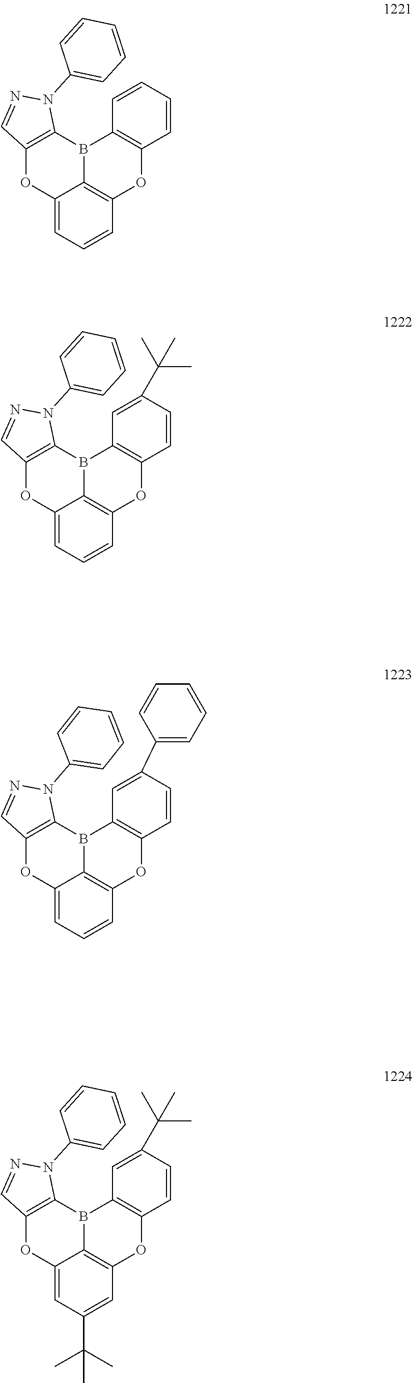 [SMILES: c1ccc(-n2ncc3c2B2c4ccccc4Oc4cccc(c42)O3)cc1 ; CC(C)(C)c1ccc2c(c1)B1c3c(cccc3Oc3cnn(-c4ccccc4)c31)O2 ; c1ccc(-c2ccc3c(c2)B2c4c(cccc4Oc4cnn(-c5ccccc5)c42)O3)cc1 ; CC(C)(C)c1cc2c3c(c1)Oc1cnn(-c4ccccc4)c1B3c1cc(C(C)(C)C)ccc1O2]

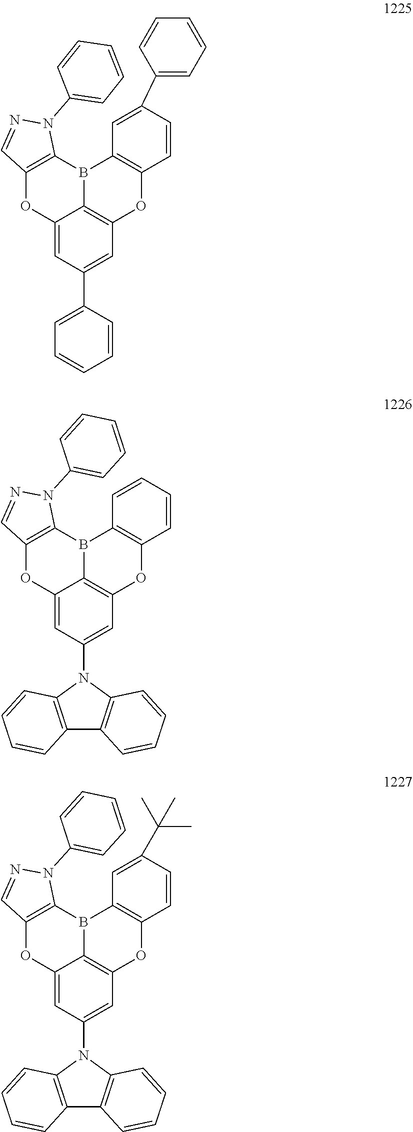

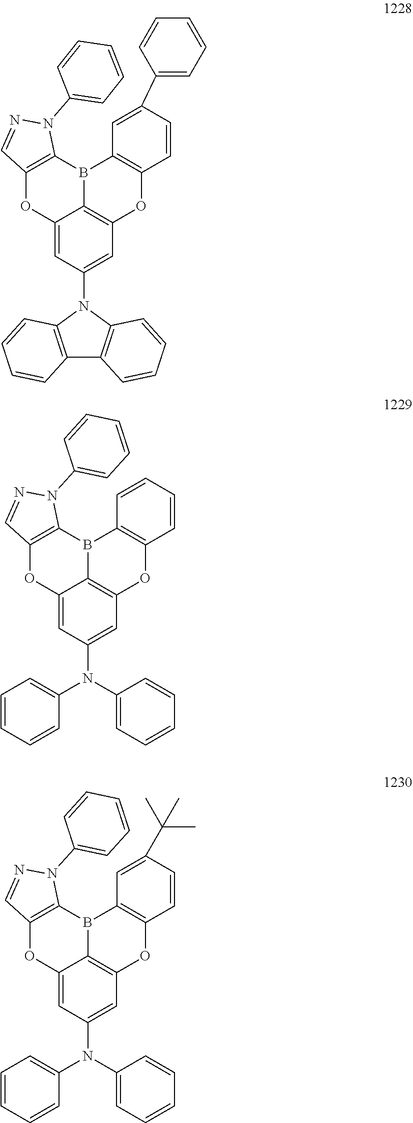

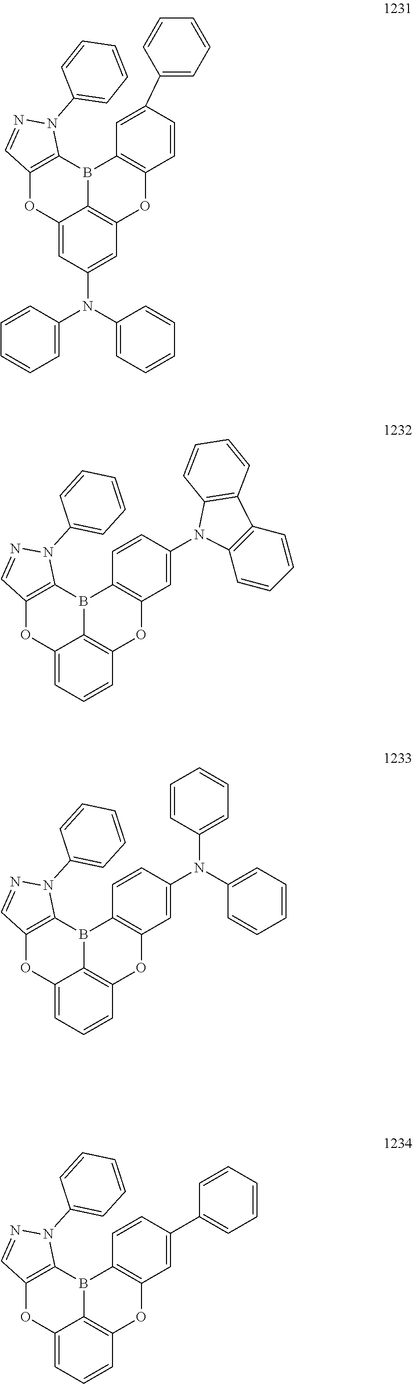 [SMILES: c1ccc(-c2ccc3c(c2)B2c4c(cc(N(c5ccccc5)c5ccccc5)cc4Oc4cnn(-c5ccccc5)c42)O3)cc1 ; c1ccc(-n2ncc3c2B2c4ccc(-n5c6ccccc6c6ccccc65)cc4Oc4cccc(c42)O3)cc1 ; c1ccc(N(c2ccccc2)c2ccc3c(c2)Oc2cccc4c2B3c2c(cnn2-c2ccccc2)O4)cc1 ; c1ccc(-c2ccc3c(c2)Oc2cccc4c2B3c2c(cnn2-c2ccccc2)O4)cc1]

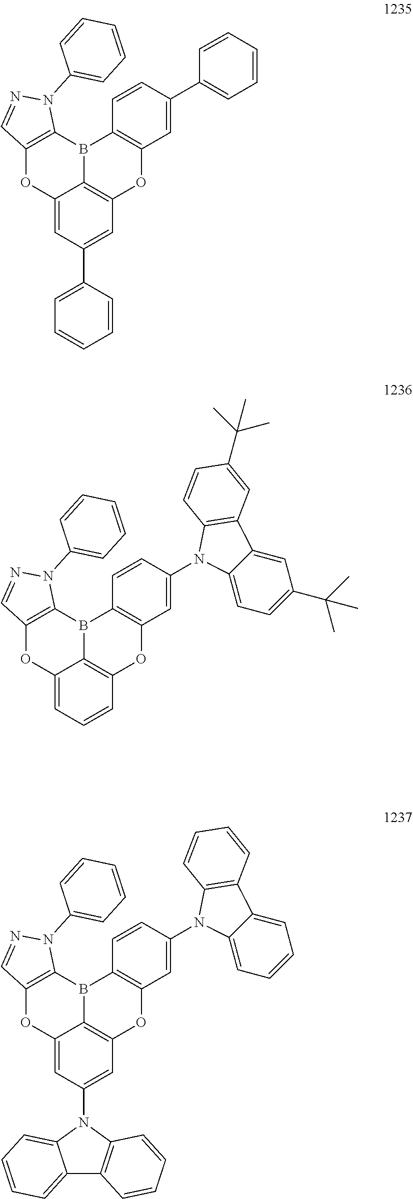 [SMILES: c1ccc(-c2ccc3c(c2)Oc2cc(-c4ccccc4)cc4c2B3c2c(cnn2-c2ccccc2)O4)cc1 ; CC(C)(C)c1ccc2c(c1)c1cc(C(C)(C)C)ccc1n2-c1ccc2c(c1)Oc1cccc3c1B2c1c(cnn1-c1ccccc1)O3 ; c1ccc(-n2ncc3c2B2c4ccc(-n5c6ccccc6c6ccccc65)cc4Oc4cc(-n5c6ccccc6c6ccccc65)cc(c42)O3)cc1]

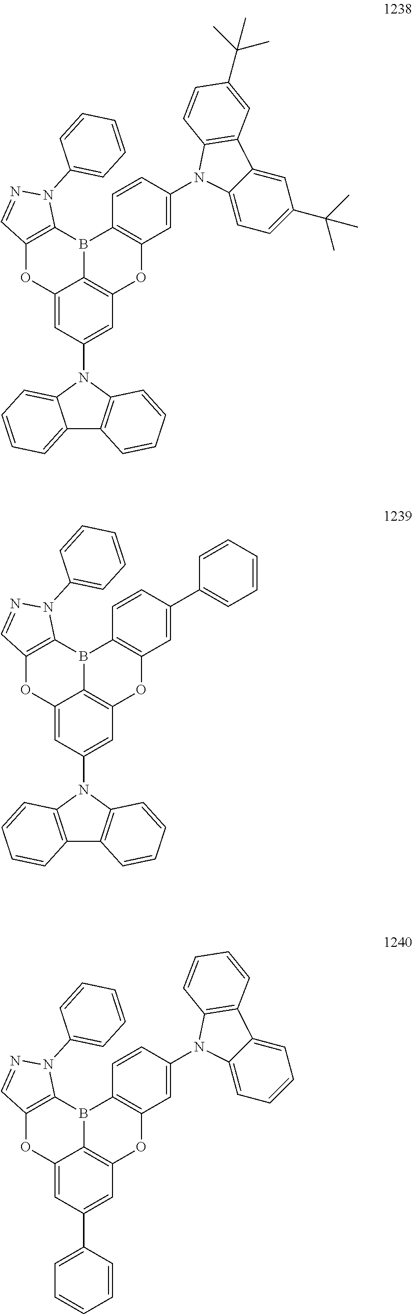 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(cnn1-c1ccccc1)O3 ; c1ccc(-c2ccc3c(c2)Oc2cc(-n4c5ccccc5c5ccccc54)cc4c2B3c2c(cnn2-c2ccccc2)O4)cc1 ; c1ccc(-c2cc3c4c(c2)Oc2cnn(-c5ccccc5)c2B4c2ccc(-n4c5ccccc5c5ccccc54)cc2O3)cc1]

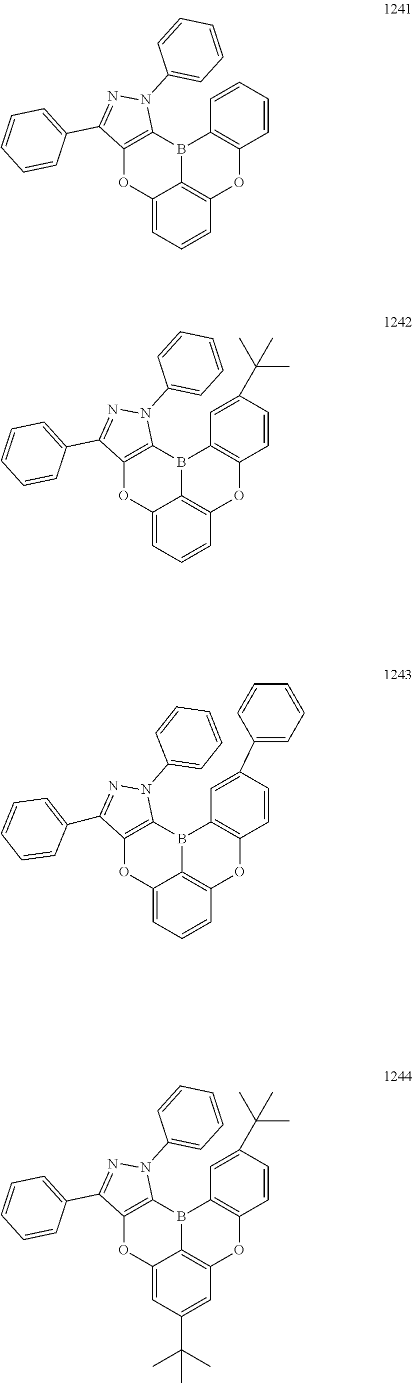 [SMILES: c1ccc(-c2nn(-c3ccccc3)c3c2Oc2cccc4c2B3c2ccccc2O4)cc1 ; CC(C)(C)c1ccc2c(c1)B1c3c(cccc3Oc3c(-c4ccccc4)nn(-c4ccccc4)c31)O2 ; c1ccc(-c2ccc3c(c2)B2c4c(cccc4Oc4c(-c5ccccc5)nn(-c5ccccc5)c42)O3)cc1 ; CC(C)(C)c1cc2c3c(c1)Oc1c(-c4ccccc4)nn(-c4ccccc4)c1B3c1cc(C(C)(C)C)ccc1O2]

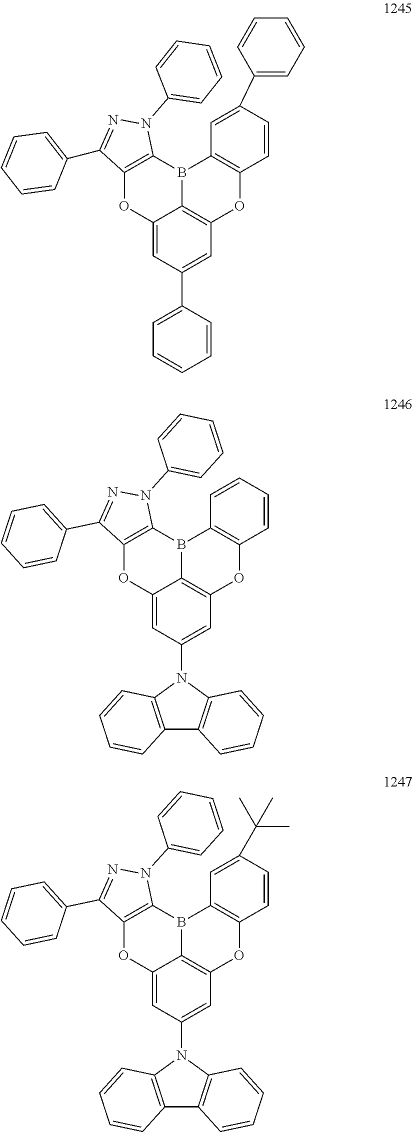

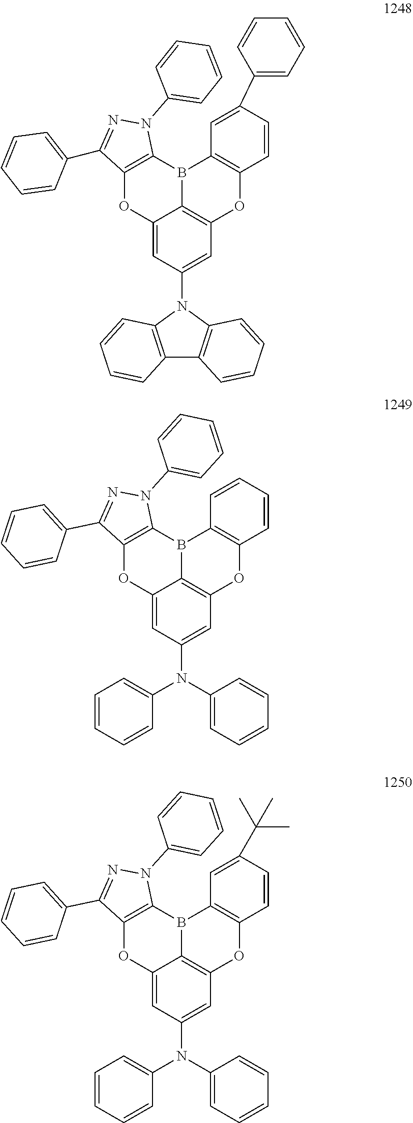

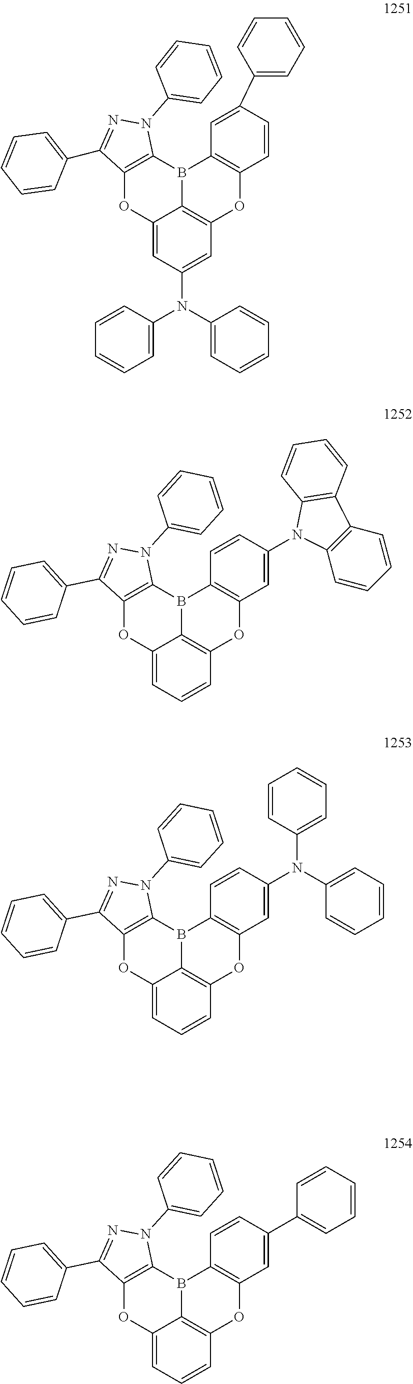 [SMILES: c1ccc(-c2ccc3c(c2)B2c4c(cc(N(c5ccccc5)c5ccccc5)cc4Oc4c(-c5ccccc5)nn(-c5ccccc5)c42)O3)cc1 ; c1ccc(-c2nn(-c3ccccc3)c3c2Oc2cccc4c2B3c2ccc(-n3c5ccccc5c5ccccc53)cc2O4)cc1 ; c1ccc(-c2nn(-c3ccccc3)c3c2Oc2cccc4c2B3c2ccc(N(c3ccccc3)c3ccccc3)cc2O4)cc1 ; c1ccc(-c2ccc3c(c2)Oc2cccc4c2B3c2c(c(-c3ccccc3)nn2-c2ccccc2)O4)cc1]

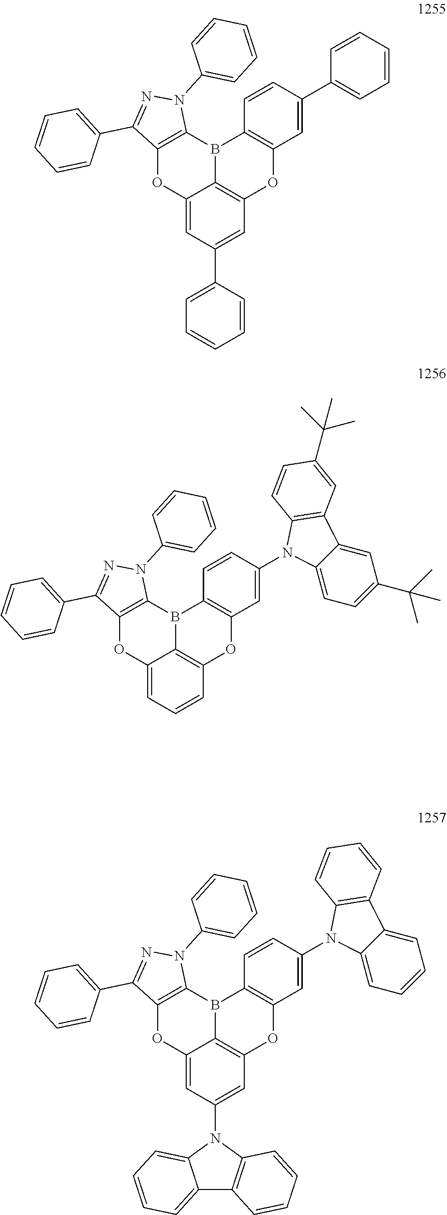 [SMILES: c1ccc(-c2ccc3c(c2)Oc2cc(-c4ccccc4)cc4c2B3c2c(c(-c3ccccc3)nn2-c2ccccc2)O4)cc1 ; CC(C)(C)c1ccc2c(c1)c1cc(C(C)(C)C)ccc1n2-c1ccc2c(c1)Oc1cccc3c1B2c1c(c(-c2ccccc2)nn1-c1ccccc1)O3 ; c1ccc(-c2nn(-c3ccccc3)c3c2Oc2cc(-n4c5ccccc5c5ccccc54)cc4c2B3c2ccc(-n3c5ccccc5c5ccccc53)cc2O4)cc1]

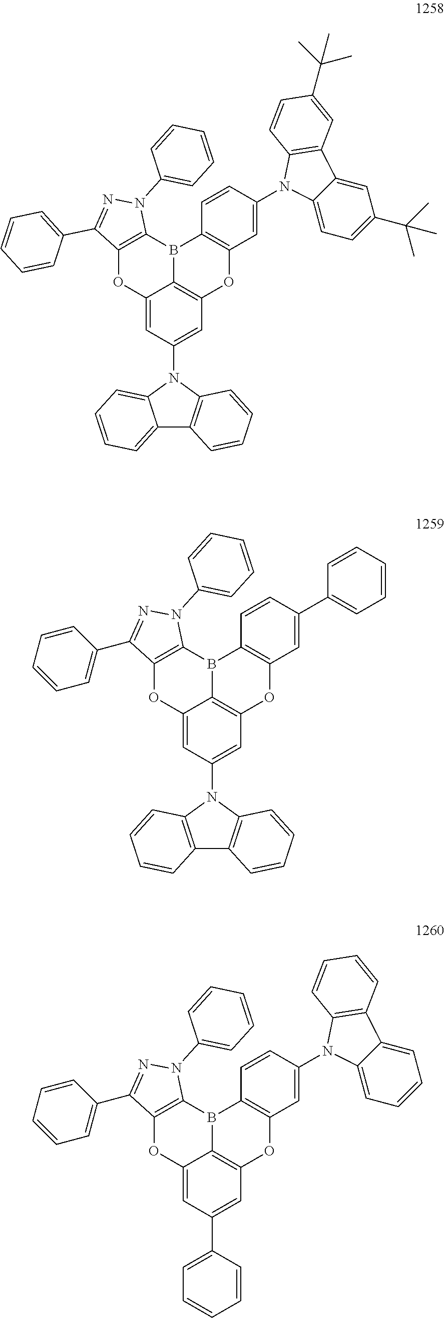 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(c(-c2ccccc2)nn1-c1ccccc1)O3 ; c1ccc(-c2ccc3c(c2)Oc2cc(-n4c5ccccc5c5ccccc54)cc4c2B3c2c(c(-c3ccccc3)nn2-c2ccccc2)O4)cc1 ; c1ccc(-c2cc3c4c(c2)Oc2c(-c5ccccc5)nn(-c5ccccc5)c2B4c2ccc(-n4c5ccccc5c5ccccc54)cc2O3)cc1]

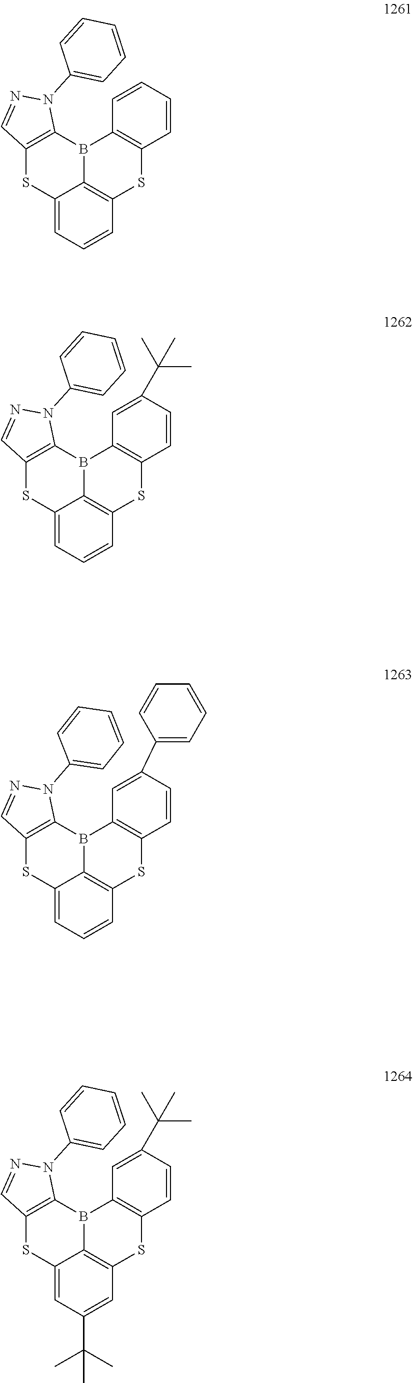 [SMILES: c1ccc(-n2ncc3c2B2c4ccccc4Sc4cccc(c42)S3)cc1 ; CC(C)(C)c1ccc2c(c1)B1c3c(cccc3Sc3cnn(-c4ccccc4)c31)S2 ; c1ccc(-c2ccc3c(c2)B2c4c(cccc4Sc4cnn(-c5ccccc5)c42)S3)cc1 ; CC(C)(C)c1cc2c3c(c1)Sc1cnn(-c4ccccc4)c1B3c1cc(C(C)(C)C)ccc1S2]

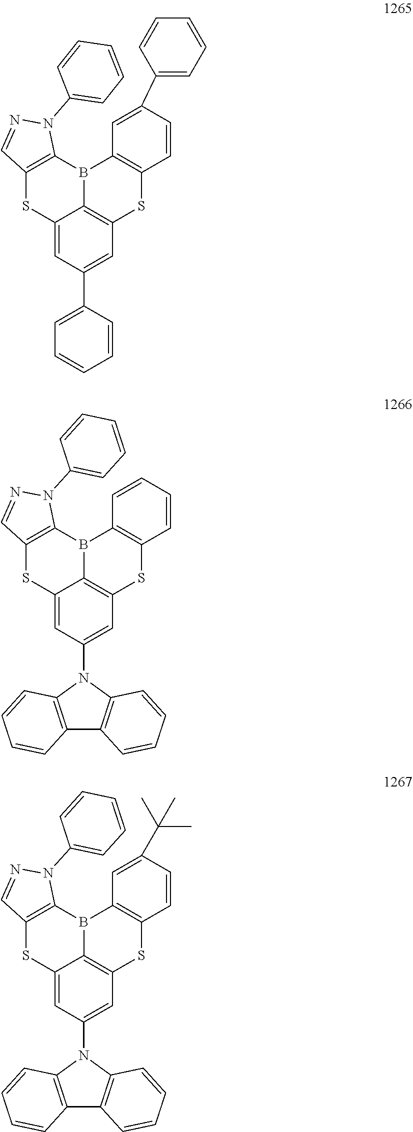

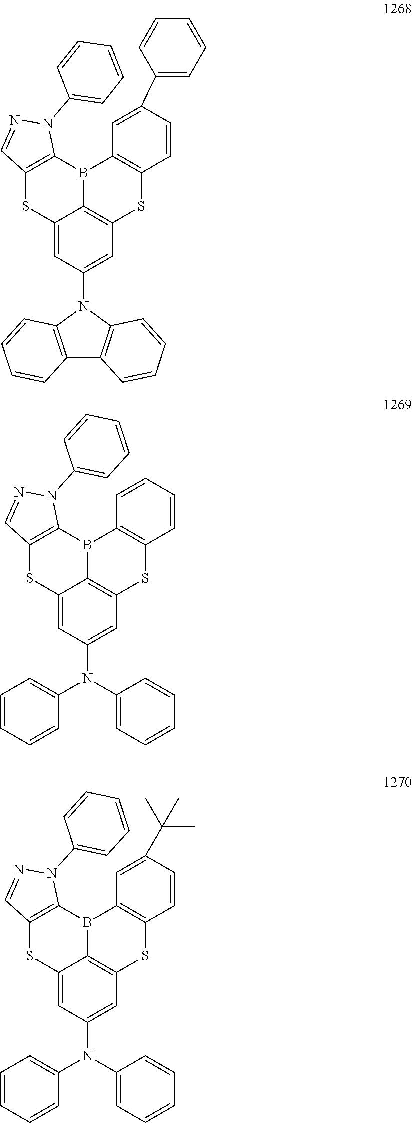

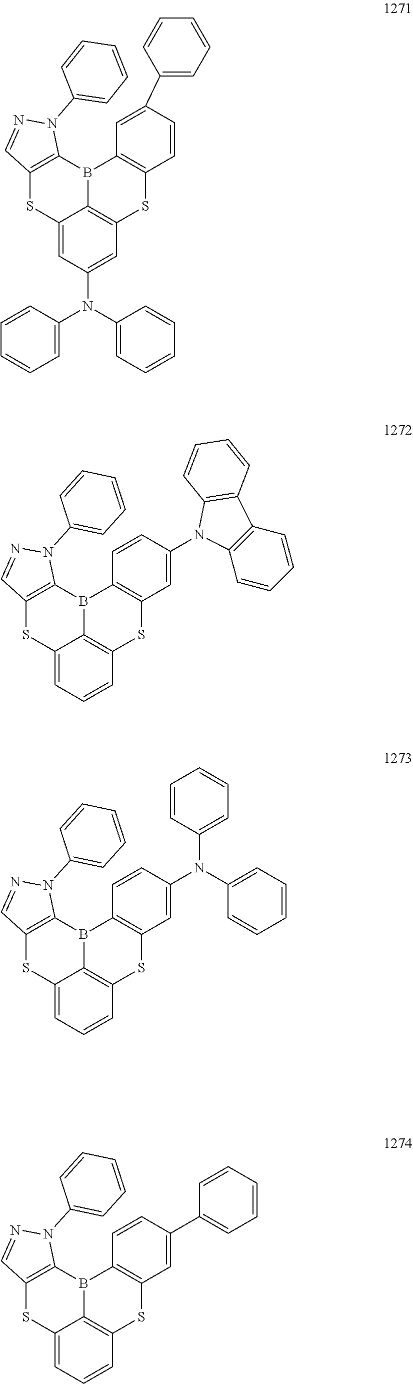 [SMILES: c1ccc(-c2ccc3c(c2)B2c4c(cc(N(c5ccccc5)c5ccccc5)cc4Sc4cnn(-c5ccccc5)c42)S3)cc1 ; c1ccc(-n2ncc3c2B2c4ccc(-n5c6ccccc6c6ccccc65)cc4Sc4cccc(c42)S3)cc1 ; c1ccc(N(c2ccccc2)c2ccc3c(c2)Sc2cccc4c2B3c2c(cnn2-c2ccccc2)S4)cc1 ; c1ccc(-c2ccc3c(c2)Sc2cccc4c2B3c2c(cnn2-c2ccccc2)S4)cc1]

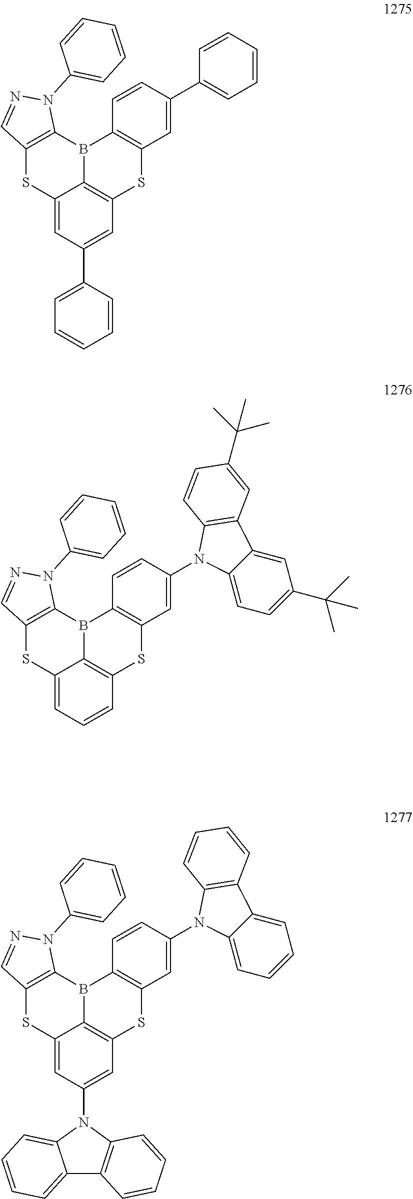 [SMILES: c1ccc(-c2ccc3c(c2)Sc2cc(-c4ccccc4)cc4c2B3c2c(cnn2-c2ccccc2)S4)cc1 ; CC(C)(C)c1ccc2c(c1)c1cc(C(C)(C)C)ccc1n2-c1ccc2c(c1)Sc1cccc3c1B2c1c(cnn1-c1ccccc1)S3 ; c1ccc(-n2ncc3c2B2c4ccc(-n5c6ccccc6c6ccccc65)cc4Sc4cc(-n5c6ccccc6c6ccccc65)cc(c42)S3)cc1]

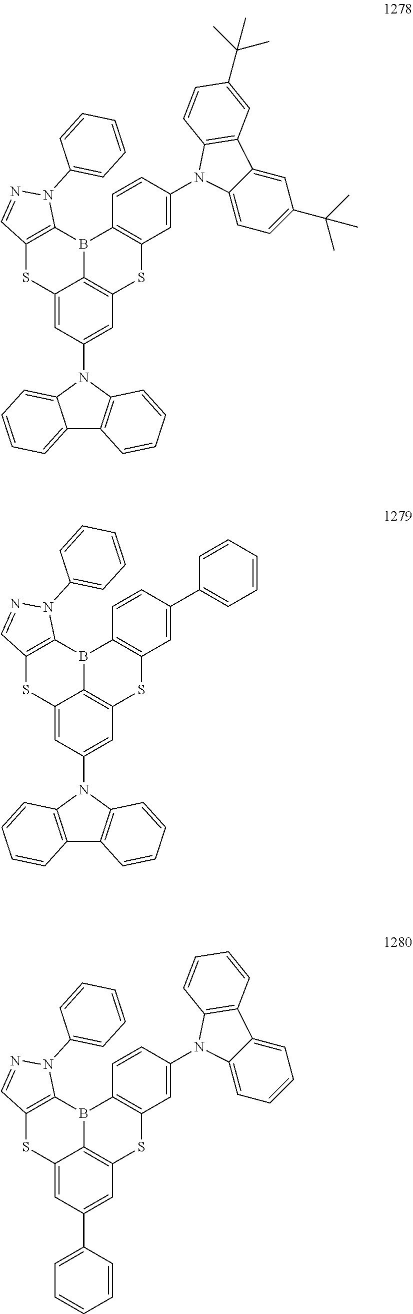 [SMILES: CC(C)(C)c1ccc2c(c1)c1cc(C(C)(C)C)ccc1n2-c1ccc2c(c1)Sc1cc(-n3c4ccccc4c4ccccc43)cc3c1B2c1c(cnn1-c1ccccc1)S3 ; c1ccc(-c2ccc3c(c2)Sc2cc(-n4c5ccccc5c5ccccc54)cc4c2B3c2c(cnn2-c2ccccc2)S4)cc1 ; c1ccc(-c2cc3c4c(c2)Sc2cnn(-c5ccccc5)c2B4c2ccc(-n4c5ccccc5c5ccccc54)cc2S3)cc1]

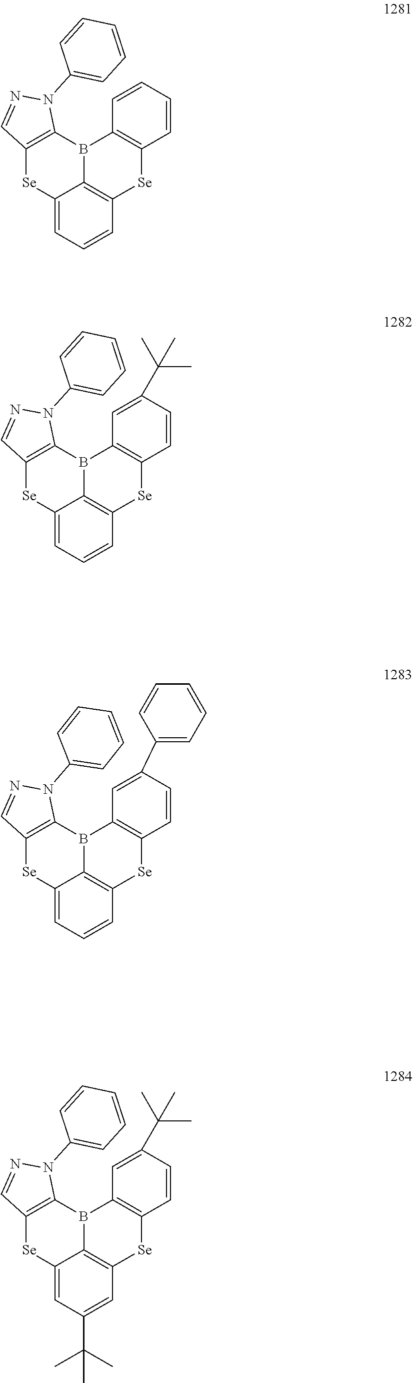 [SMILES: c1ccc(-n2ncc3c2B2c4ccccc4[Se]c4cccc(c42)[Se]3)cc1 ; CC(C)(C)c1ccc2c(c1)B1c3c(cccc3[Se]c3cnn(-c4ccccc4)c31)[Se]2 ; c1ccc(-c2ccc3c(c2)B2c4c(cccc4[Se]c4cnn(-c5ccccc5)c42)[Se]3)cc1 ; CC(C)(C)c1cc2c3c(c1)[Se]c1cnn(-c4ccccc4)c1B3c1cc(C(C)(C)C)ccc1[Se]2]

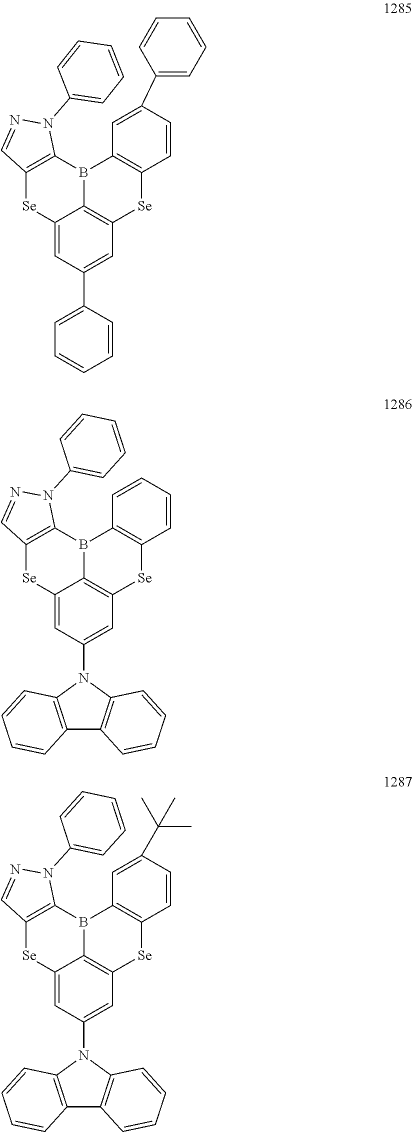

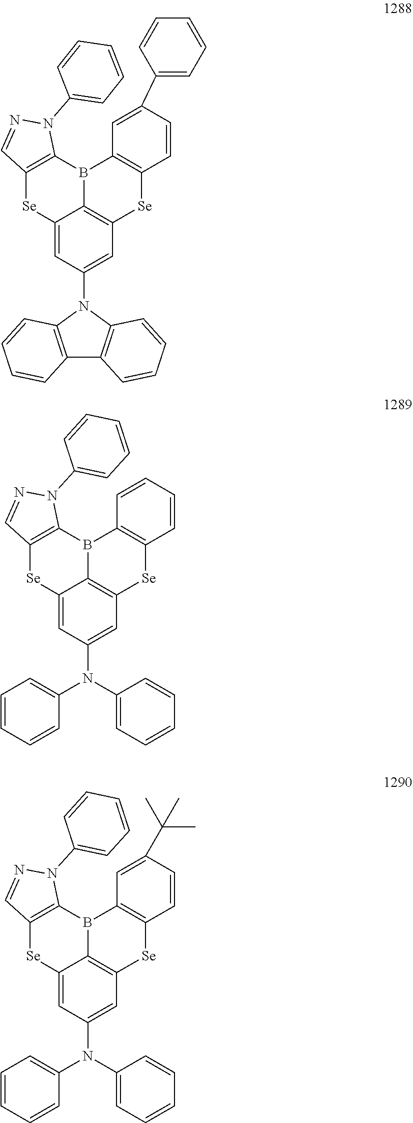

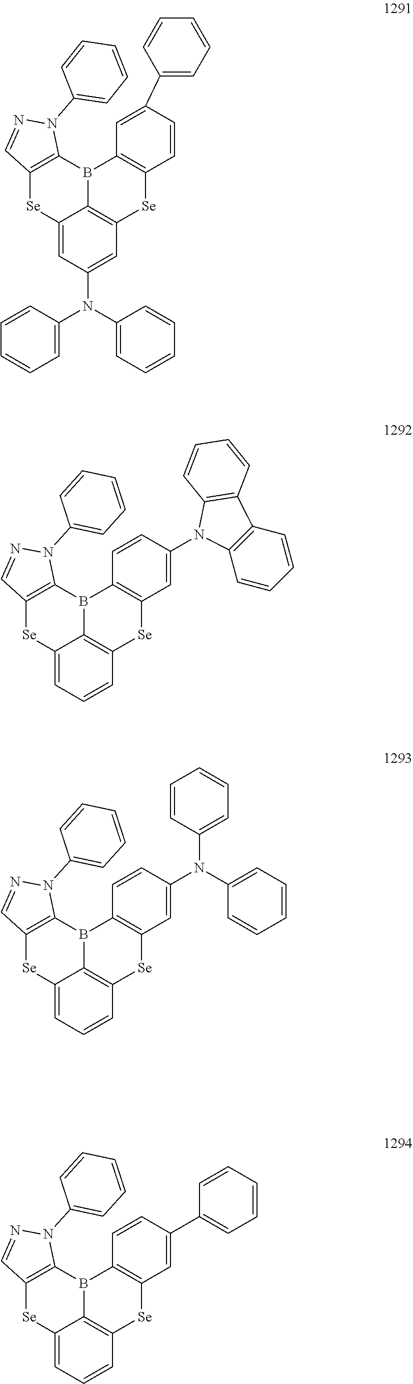 [SMILES: c1ccc(-c2ccc3c(c2)B2c4c(cc(N(c5ccccc5)c5ccccc5)cc4[Se]c4cnn(-c5ccccc5)c42)[Se]3)cc1 ; c1ccc(-n2ncc3c2B2c4ccc(-n5c6ccccc6c6ccccc65)cc4[Se]c4cccc(c42)[Se]3)cc1 ; c1ccc(N(c2ccccc2)c2ccc3c(c2)[Se]c2cccc4c2B3c2c(cnn2-c2ccccc2)[Se]4)cc1 ; c1ccc(-c2ccc3c(c2)[Se]c2cccc4c2B3c2c(cnn2-c2ccccc2)[Se]4)cc1]

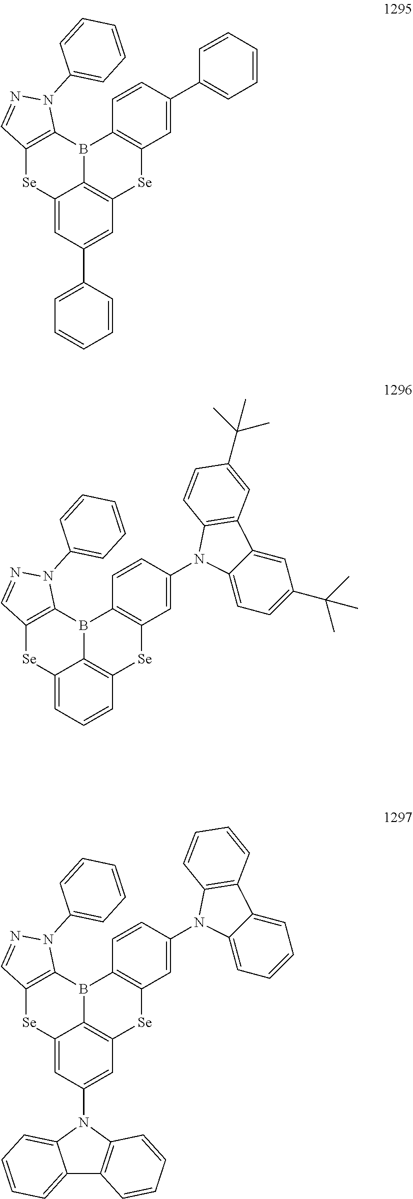 [SMILES: c1ccc(-c2ccc3c(c2)[Se]c2cc(-c4ccccc4)cc4c2B3c2c(cnn2-c2ccccc2)[Se]4)cc1 ; CC(C)(C)c1ccc2c(c1)c1cc(C(C)(C)C)ccc1n2-c1ccc2c(c1)[Se]c1cccc3c1B2c1c(cnn1-c1ccccc1)[Se]3 ; c1ccc(-n2ncc3c2B2c4ccc(-n5c6ccccc6c6ccccc65)cc4[Se]c4cc(-n5c6ccccc6c6ccccc65)cc(c42)[Se]3)cc1]

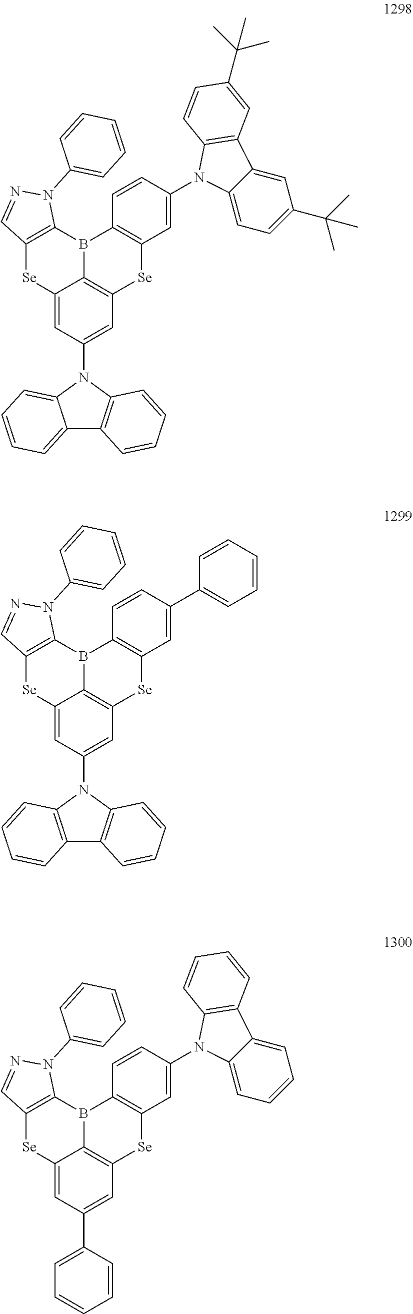 [SMILES: CC(C)(C)c1ccc2c(c1)c1cc(C(C)(C)C)ccc1n2-c1ccc2c(c1)[Se]c1cc(-n3c4ccccc4c4ccccc43)cc3c1B2c1c(cnn1-c1ccccc1)[Se]3 ; c1ccc(-c2ccc3c(c2)[Se]c2cc(-n4c5ccccc5c5ccccc54)cc4c2B3c2c(cnn2-c2ccccc2)[Se]4)cc1 ; c1ccc(-c2cc3c4c(c2)[Se]c2cnn(-c5ccccc5)c2B4c2ccc(-n4c5ccccc5c5ccccc54)cc2[Se]3)cc1]

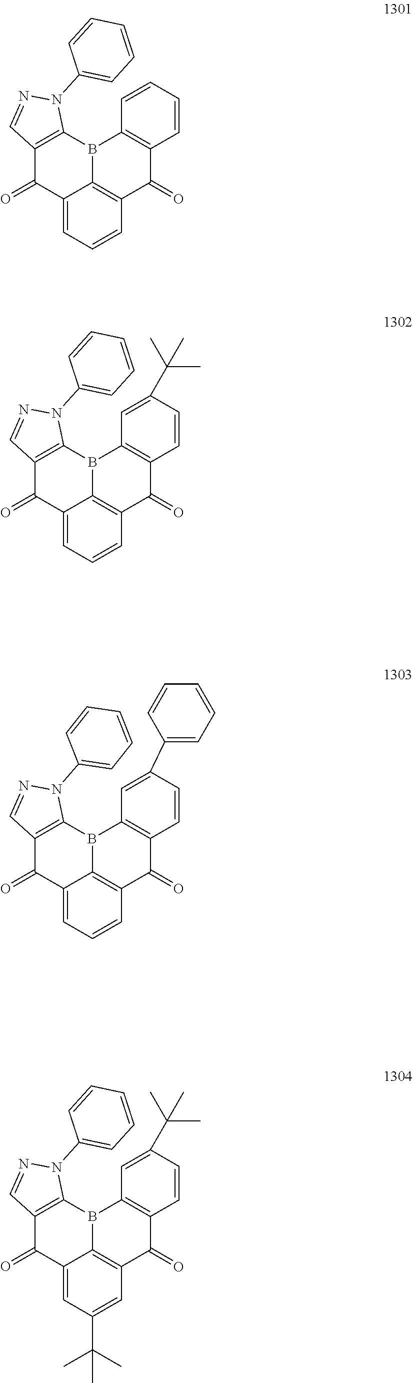 [SMILES: O=C1c2ccccc2B2c3c1cccc3C(=O)c1cnn(-c3ccccc3)c12 ; CC(C)(C)c1ccc2c(c1)B1c3c(cccc3C(=O)c3cnn(-c4ccccc4)c31)C2=O ; O=C1c2ccc(-c3ccccc3)cc2B2c3c1cccc3C(=O)c1cnn(-c3ccccc3)c12 ; CC(C)(C)c1ccc2c(c1)B1c3c(cc(C(C)(C)C)cc3C(=O)c3cnn(-c4ccccc4)c31)C2=O]

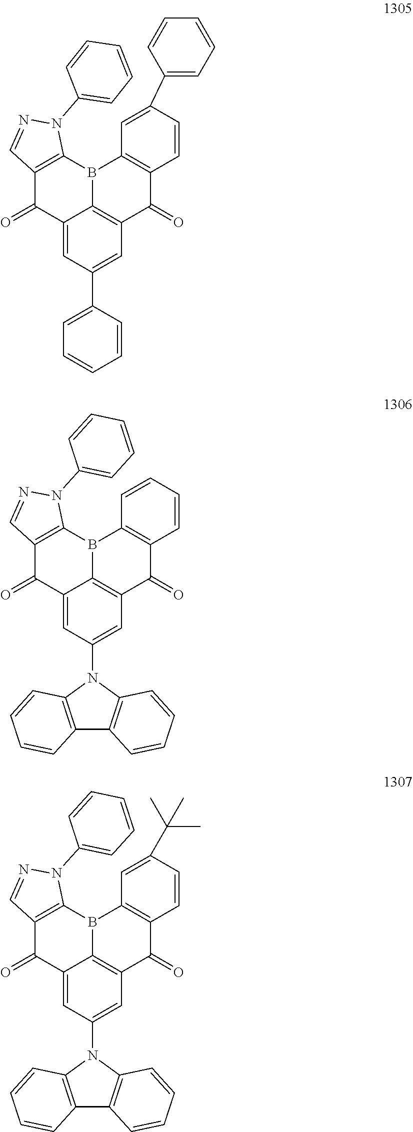

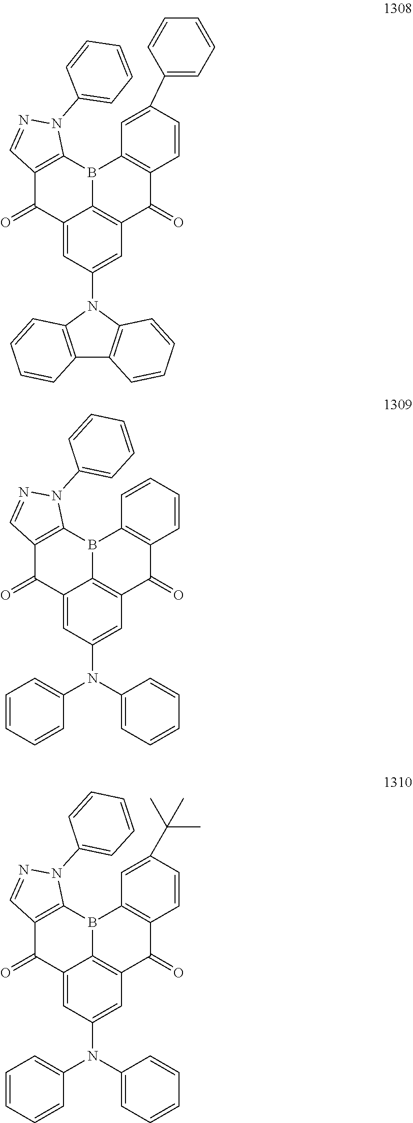

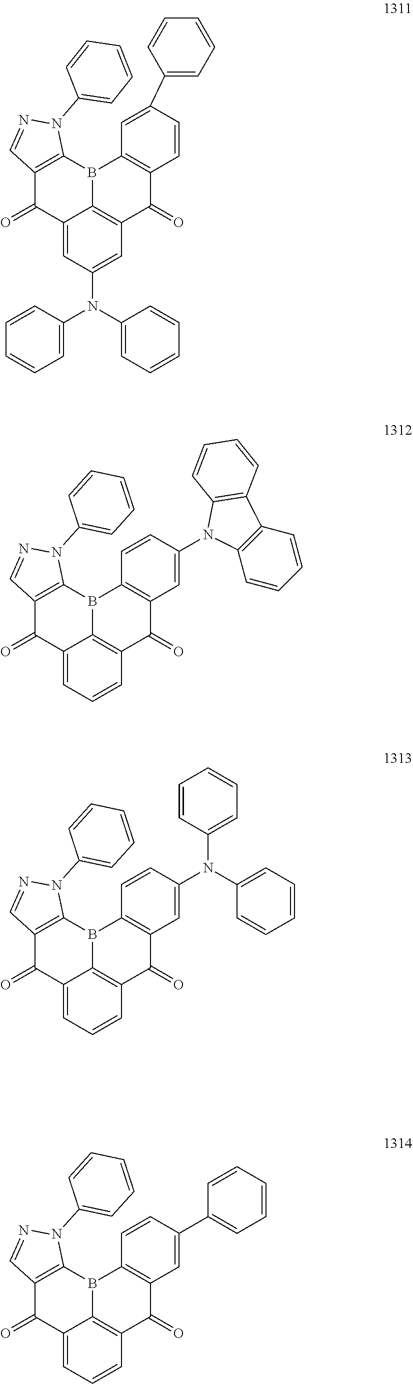 [SMILES: O=C1c2ccc(-c3ccccc3)cc2B2c3c1cc(N(c1ccccc1)c1ccccc1)cc3C(=O)c1cnn(-c3ccccc3)c12 ; O=C1c2cc(-n3c4ccccc4c4ccccc43)ccc2B2c3c1cccc3C(=O)c1cnn(-c3ccccc3)c12 ; O=C1c2cc(N(c3ccccc3)c3ccccc3)ccc2B2c3c1cccc3C(=O)c1cnn(-c3ccccc3)c12 ; O=C1c2cc(-c3ccccc3)ccc2B2c3c1cccc3C(=O)c1cnn(-c3ccccc3)c12]

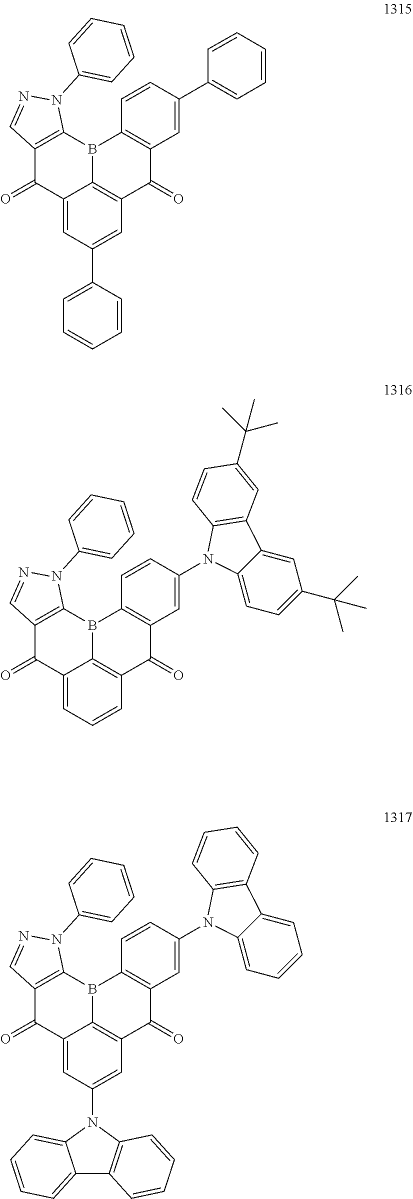 [SMILES: O=C1c2cc(-c3ccccc3)ccc2B2c3c1cc(-c1ccccc1)cc3C(=O)c1cnn(-c3ccccc3)c12 ; CC(C)(C)c1ccc2c(c1)c1cc(C(C)(C)C)ccc1n2-c1ccc2c(c1)C(=O)c1cccc3c1B2c1c(cnn1-c1ccccc1)C3=O ; O=C1c2cc(-n3c4ccccc4c4ccccc43)ccc2B2c3c1cc(-n1c4ccccc4c4ccccc41)cc3C(=O)c1cnn(-c3ccccc3)c12]

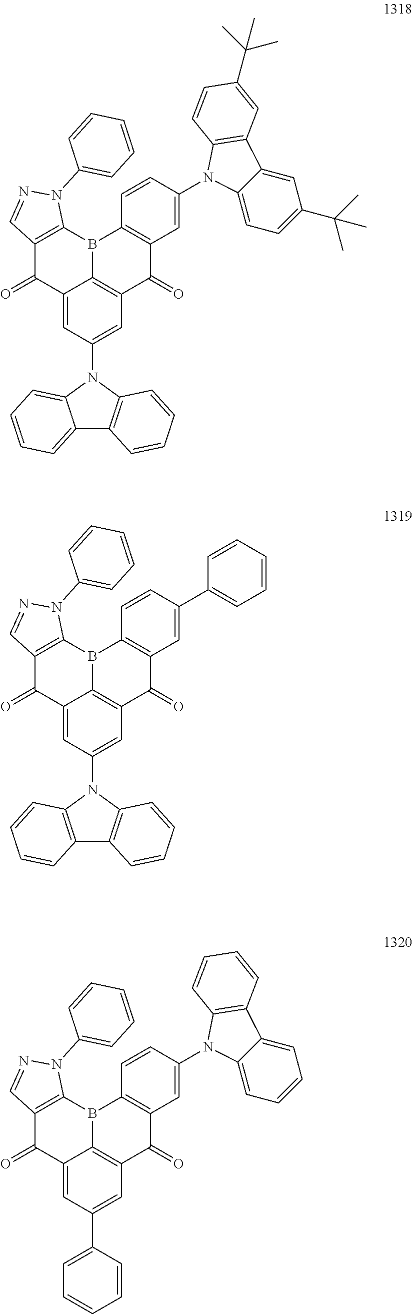 [SMILES: CC(C)(C)c1ccc2c(c1)c1cc(C(C)(C)C)ccc1n2-c1ccc2c(c1)C(=O)c1cc(-n3c4ccccc4c4ccccc43)cc3c1B2c1c(cnn1-c1ccccc1)C3=O ; O=C1c2cc(-c3ccccc3)ccc2B2c3c1cc(-n1c4ccccc4c4ccccc41)cc3C(=O)c1cnn(-c3ccccc3)c12 ; O=C1c2cc(-n3c4ccccc4c4ccccc43)ccc2B2c3c1cc(-c1ccccc1)cc3C(=O)c1cnn(-c3ccccc3)c12]

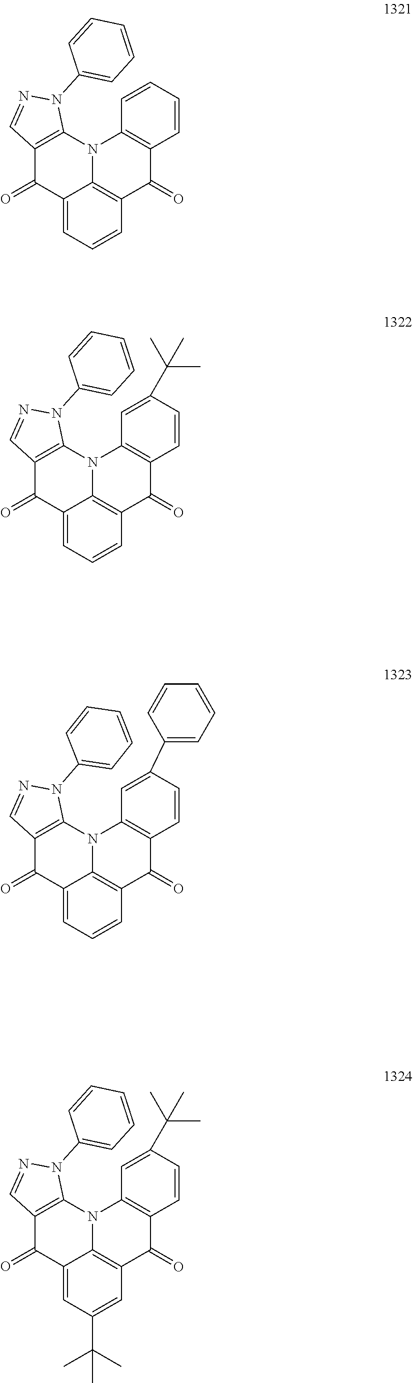 [SMILES: O=c1c2ccccc2n2c3c1cccc3c(=O)c1cnn(-c3ccccc3)c12 ; CC(C)(C)c1ccc2c(=O)c3cccc4c(=O)c5cnn(-c6ccccc6)c5n(c2c1)c34 ; O=c1c2ccc(-c3ccccc3)cc2n2c3c1cccc3c(=O)c1cnn(-c3ccccc3)c12 ; CC(C)(C)c1cc2c(=O)c3ccc(C(C)(C)C)cc3n3c2c(c1)c(=O)c1cnn(-c2ccccc2)c13]

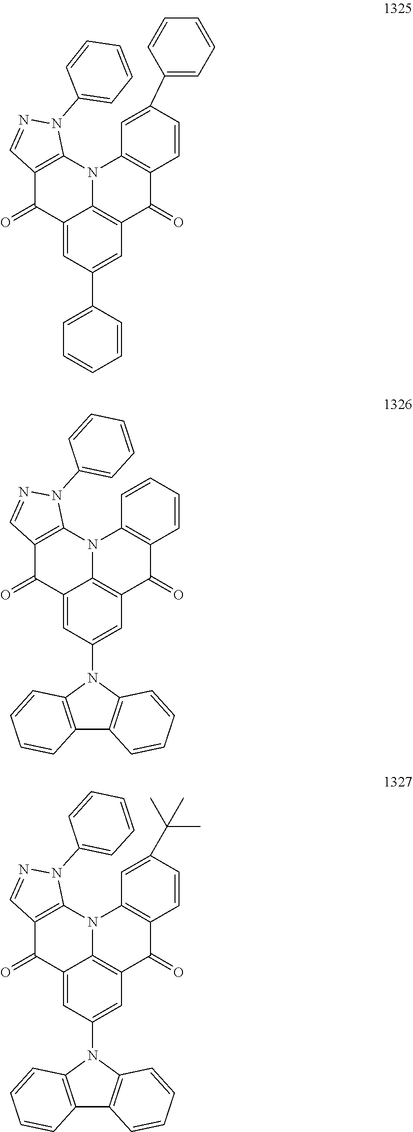

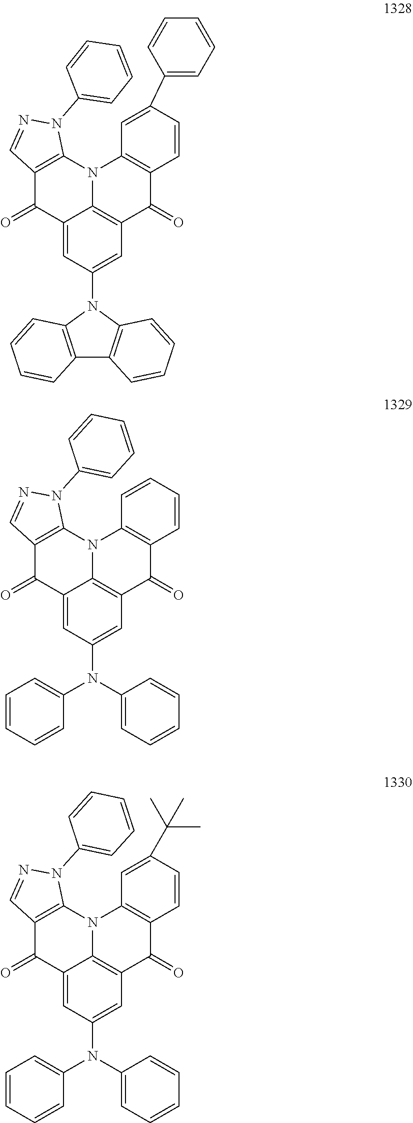

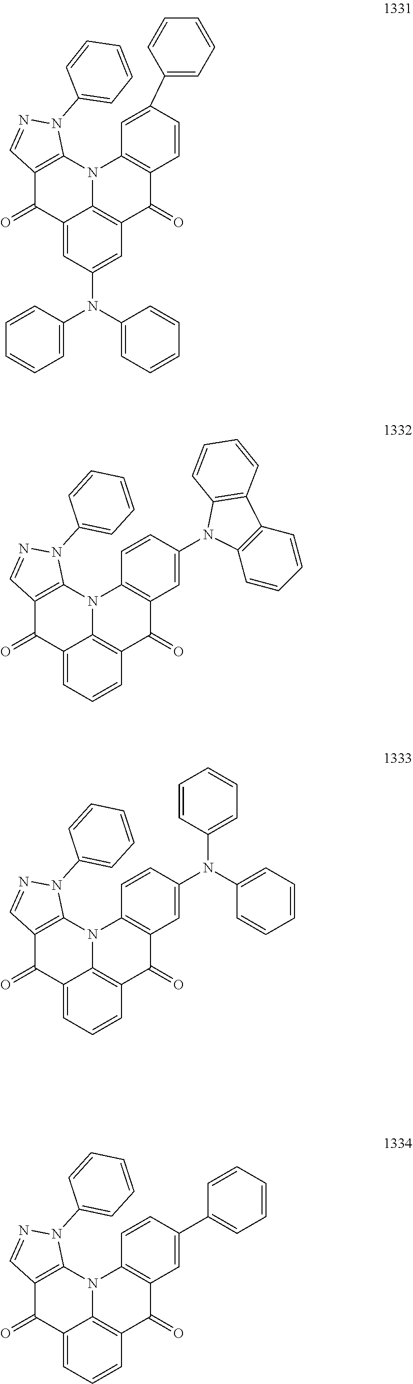 [SMILES: O=c1c2ccc(-c3ccccc3)cc2n2c3c1cc(N(c1ccccc1)c1ccccc1)cc3c(=O)c1cnn(-c3ccccc3)c12 ; O=c1c2cc(-n3c4ccccc4c4ccccc43)ccc2n2c3c1cccc3c(=O)c1cnn(-c3ccccc3)c12 ; O=c1c2cc(N(c3ccccc3)c3ccccc3)ccc2n2c3c1cccc3c(=O)c1cnn(-c3ccccc3)c12 ; O=c1c2cc(-c3ccccc3)ccc2n2c3c1cccc3c(=O)c1cnn(-c3ccccc3)c12]

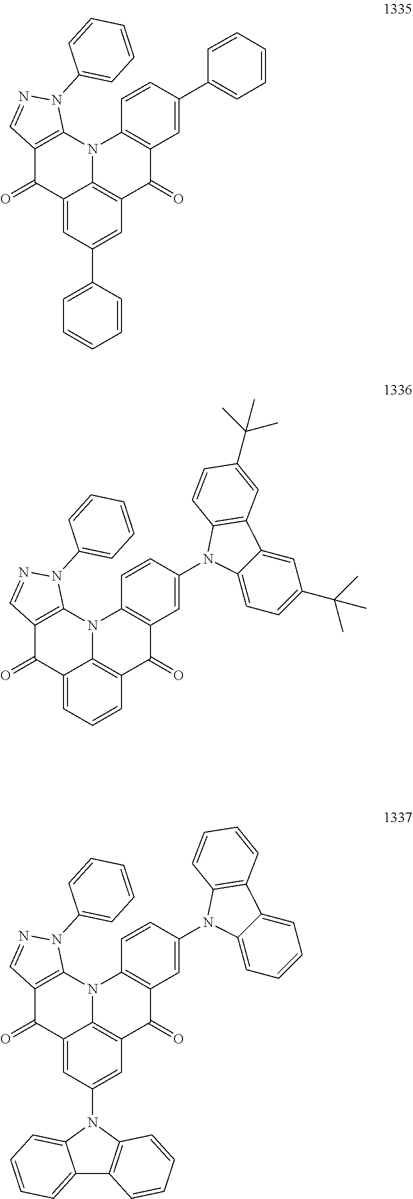 [SMILES: O=c1c2cc(-c3ccccc3)ccc2n2c3c1cc(-c1ccccc1)cc3c(=O)c1cnn(-c3ccccc3)c12 ; CC(C)(C)c1ccc2c(c1)c1cc(C(C)(C)C)ccc1n2-c1ccc2c(c1)c(=O)c1cccc3c(=O)c4cnn(-c5ccccc5)c4n2c13 ; O=c1c2cc(-n3c4ccccc4c4ccccc43)ccc2n2c3c1cc(-n1c4ccccc4c4ccccc41)cc3c(=O)c1cnn(-c3ccccc3)c12]

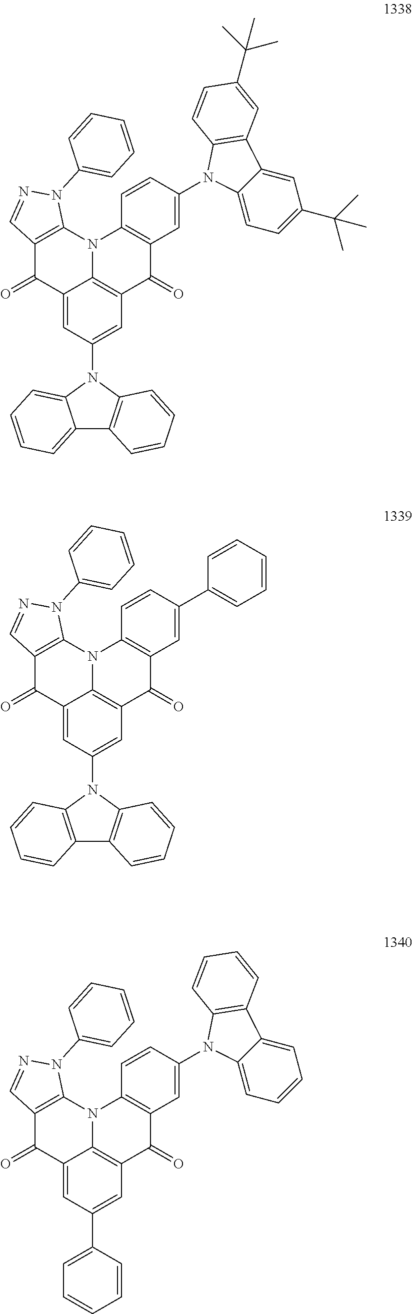 [SMILES: CC(C)(C)c1ccc2c(c1)c1cc(C(C)(C)C)ccc1n2-c1ccc2c(c1)c(=O)c1cc(-n3c4ccccc4c4ccccc43)cc3c(=O)c4cnn(-c5ccccc5)c4n2c13 ; O=c1c2cc(-c3ccccc3)ccc2n2c3c1cc(-n1c4ccccc4c4ccccc41)cc3c(=O)c1cnn(-c3ccccc3)c12 ; O=c1c2cc(-n3c4ccccc4c4ccccc43)ccc2n2c3c1cc(-c1ccccc1)cc3c(=O)c1cnn(-c3ccccc3)c12]

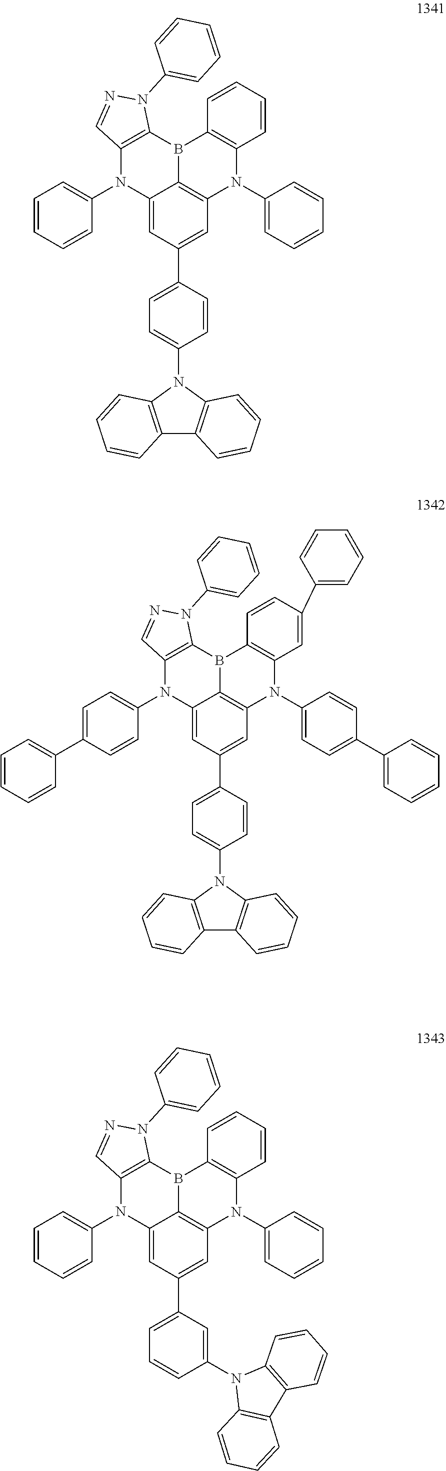 [SMILES: c1ccc(N2c3ccccc3B3c4c2cc(-c2ccc(-n5c6ccccc6c6ccccc65)cc2)cc4N(c2ccccc2)c2cnn(-c4ccccc4)c23)cc1 ; c1ccc(-c2ccc(N3c4cc(-c5ccccc5)ccc4B4c5c3cc(-c3ccc(-n6c7ccccc7c7ccccc76)cc3)cc5N(c3ccc(-c5ccccc5)cc3)c3cnn(-c5ccccc5)c34)cc2)cc1 ; c1ccc(N2c3ccccc3B3c4c2cc(-c2cccc(-n5c6ccccc6c6ccccc65)c2)cc4N(c2ccccc2)c2cnn(-c4ccccc4)c23)cc1]

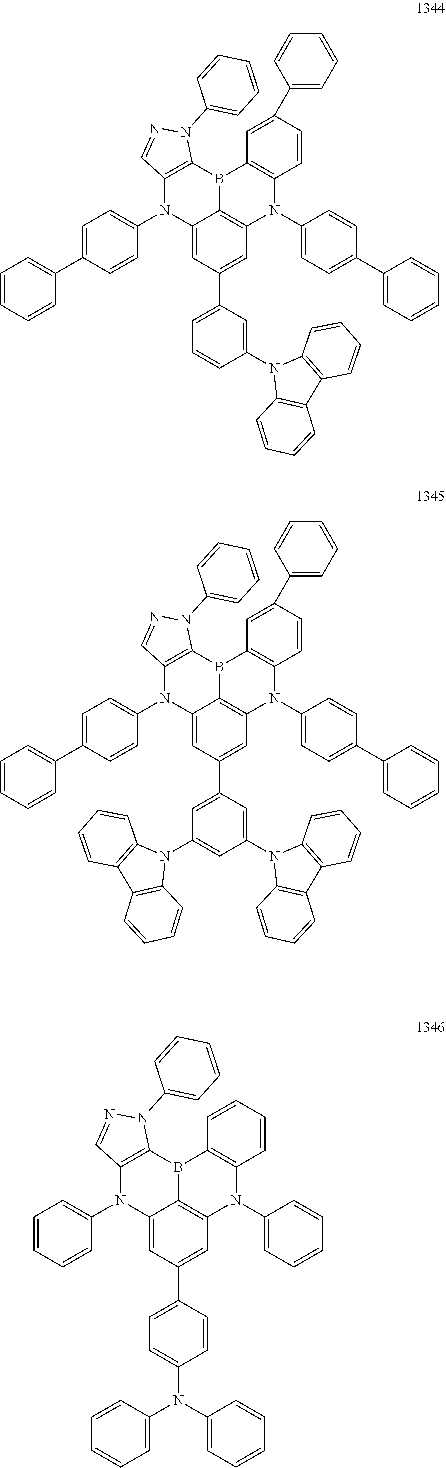 [SMILES: c1ccc(-c2ccc(N3c4ccc(-c5ccccc5)cc4B4c5c3cc(-c3cccc(-n6c7ccccc7c7ccccc76)c3)cc5N(c3ccc(-c5ccccc5)cc3)c3cnn(-c5ccccc5)c34)cc2)cc1 ; c1ccc(-c2ccc(N3c4ccc(-c5ccccc5)cc4B4c5c3cc(-c3cc(-n6c7ccccc7c7ccccc76)cc(-n6c7ccccc7c7ccccc76)c3)cc5N(c3ccc(-c5ccccc5)cc3)c3cnn(-c5ccccc5)c34)cc2)cc1 ; c1ccc(N(c2ccccc2)c2ccc(-c3cc4c5c(c3)N(c3ccccc3)c3cnn(-c6ccccc6)c3B5c3ccccc3N4c3ccccc3)cc2)cc1]

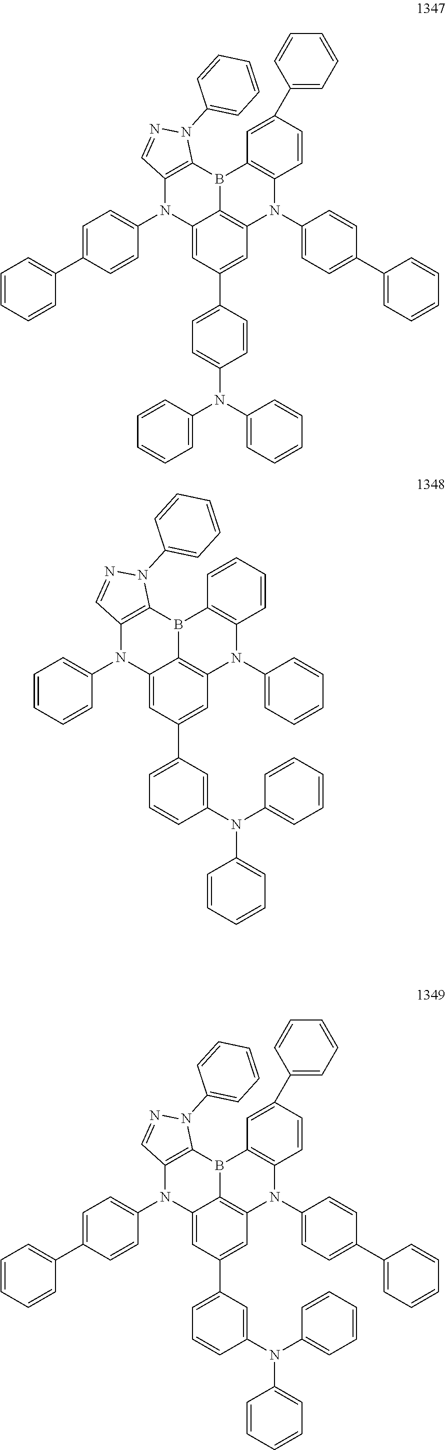

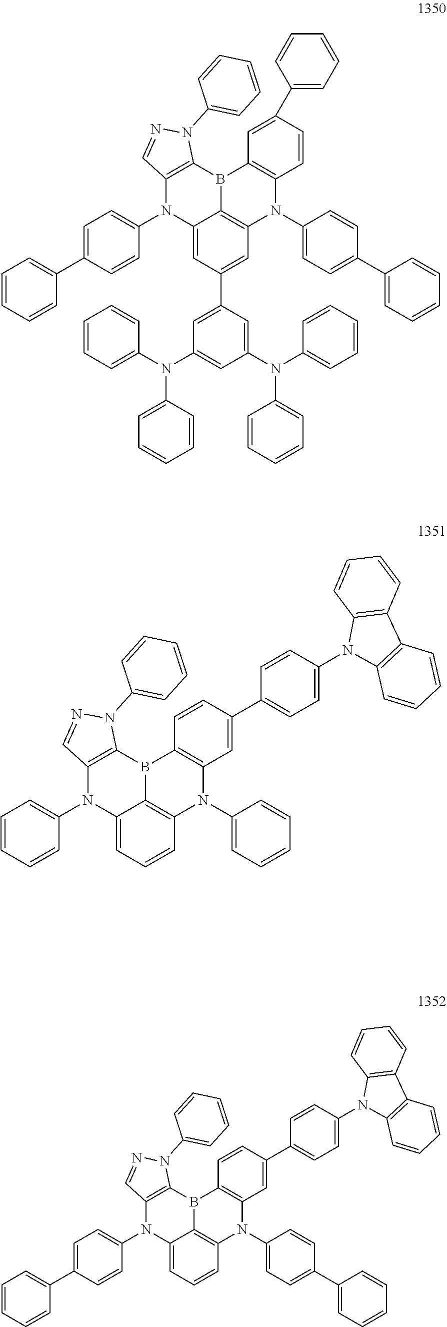 [SMILES: c1ccc(-c2ccc(N3c4ccc(-c5ccccc5)cc4B4c5c3cc(-c3cc(N(c6ccccc6)c6ccccc6)cc(N(c6ccccc6)c6ccccc6)c3)cc5N(c3ccc(-c5ccccc5)cc3)c3cnn(-c5ccccc5)c34)cc2)cc1 ; c1ccc(N2c3cc(-c4ccc(-n5c6ccccc6c6ccccc65)cc4)ccc3B3c4c2cccc4N(c2ccccc2)c2cnn(-c4ccccc4)c23)cc1 ; c1ccc(-c2ccc(N3c4cc(-c5ccc(-n6c7ccccc7c7ccccc76)cc5)ccc4B4c5c3cccc5N(c3ccc(-c5ccccc5)cc3)c3cnn(-c5ccccc5)c34)cc2)cc1]

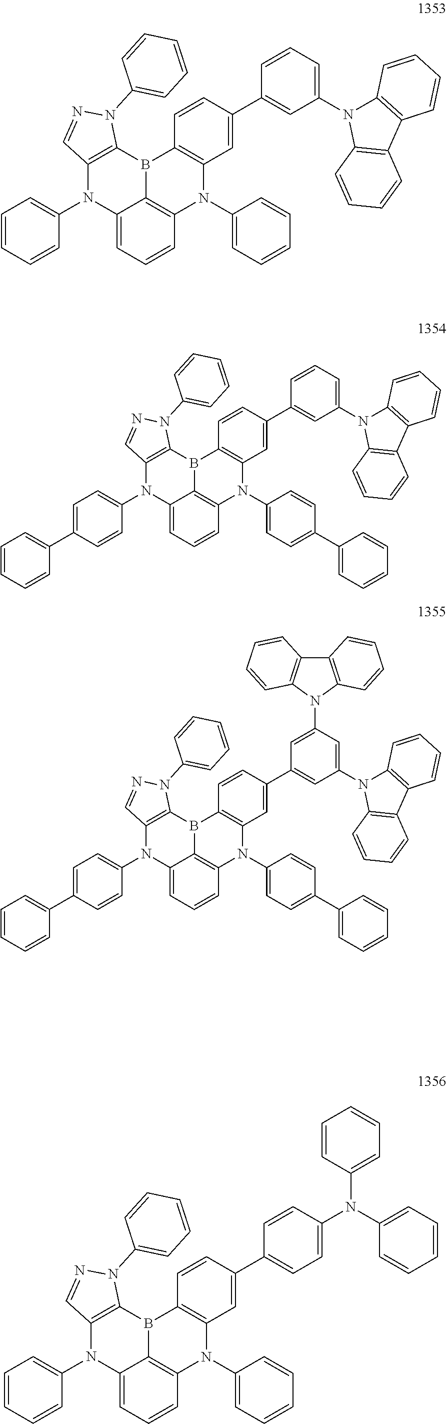 [SMILES: c1ccc(N2c3cc(-c4cccc(-n5c6ccccc6c6ccccc65)c4)ccc3B3c4c2cccc4N(c2ccccc2)c2cnn(-c4ccccc4)c23)cc1 ; c1ccc(-c2ccc(N3c4cc(-c5cccc(-n6c7ccccc7c7ccccc76)c5)ccc4B4c5c3cccc5N(c3ccc(-c5ccccc5)cc3)c3cnn(-c5ccccc5)c34)cc2)cc1 ; c1ccc(-c2ccc(N3c4cc(-c5cc(-n6c7ccccc7c7ccccc76)cc(-n6c7ccccc7c7ccccc76)c5)ccc4B4c5c3cccc5N(c3ccc(-c5ccccc5)cc3)c3cnn(-c5ccccc5)c34)cc2)cc1 ; c1ccc(N(c2ccccc2)c2ccc(-c3ccc4c(c3)N(c3ccccc3)c3cccc5c3B4c3c(cnn3-c3ccccc3)N5c3ccccc3)cc2)cc1]

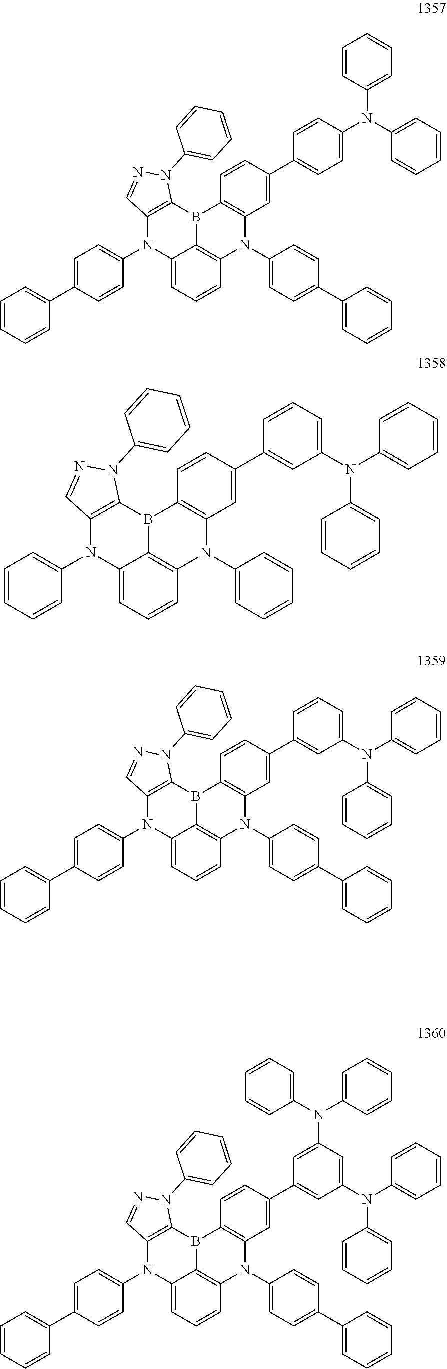 [SMILES: c1ccc(-c2ccc(N3c4cc(-c5ccc(N(c6ccccc6)c6ccccc6)cc5)ccc4B4c5c3cccc5N(c3ccc(-c5ccccc5)cc3)c3cnn(-c5ccccc5)c34)cc2)cc1 ; c1ccc(N(c2ccccc2)c2cccc(-c3ccc4c(c3)N(c3ccccc3)c3cccc5c3B4c3c(cnn3-c3ccccc3)N5c3ccccc3)c2)cc1 ; c1ccc(-c2ccc(N3c4cc(-c5cccc(N(c6ccccc6)c6ccccc6)c5)ccc4B4c5c3cccc5N(c3ccc(-c5ccccc5)cc3)c3cnn(-c5ccccc5)c34)cc2)cc1 ; c1ccc(-c2ccc(N3c4cc(-c5cc(N(c6ccccc6)c6ccccc6)cc(N(c6ccccc6)c6ccccc6)c5)ccc4B4c5c3cccc5N(c3ccc(-c5ccccc5)cc3)c3cnn(-c5ccccc5)c34)cc2)cc1]

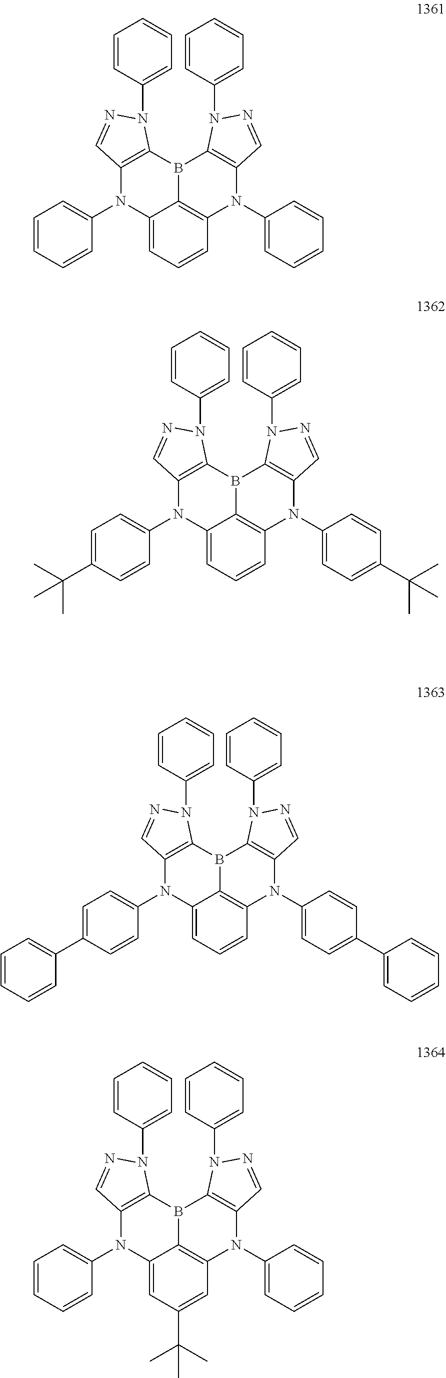 [SMILES: c1ccc(N2c3cccc4c3B(c3c2cnn3-c2ccccc2)c2c(cnn2-c2ccccc2)N4c2ccccc2)cc1 ; CC(C)(C)c1ccc(N2c3cccc4c3B(c3c2cnn3-c2ccccc2)c2c(cnn2-c2ccccc2)N4c2ccc(C(C)(C)C)cc2)cc1 ; c1ccc(-c2ccc(N3c4cccc5c4B(c4c3cnn4-c3ccccc3)c3c(cnn3-c3ccccc3)N5c3ccc(-c4ccccc4)cc3)cc2)cc1 ; CC(C)(C)c1cc2c3c(c1)N(c1ccccc1)c1cnn(-c4ccccc4)c1B3c1c(cnn1-c1ccccc1)N2c1ccccc1]

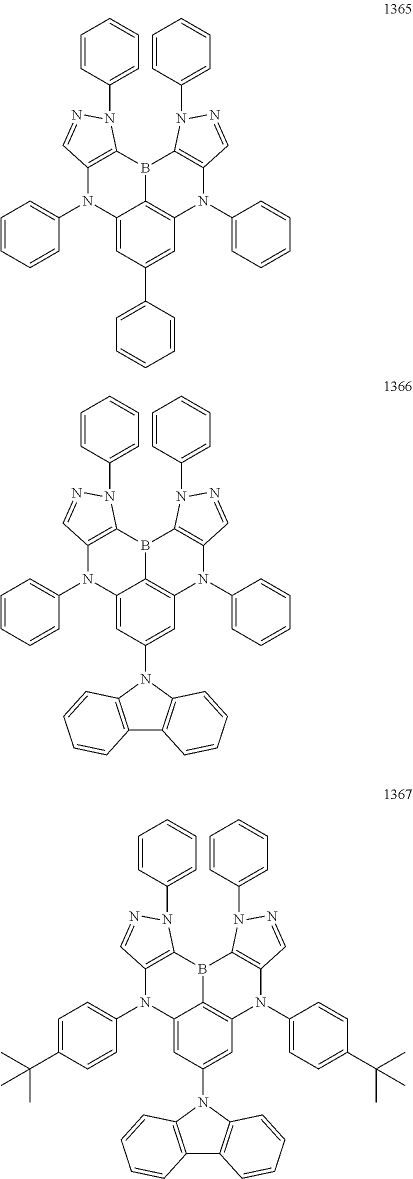

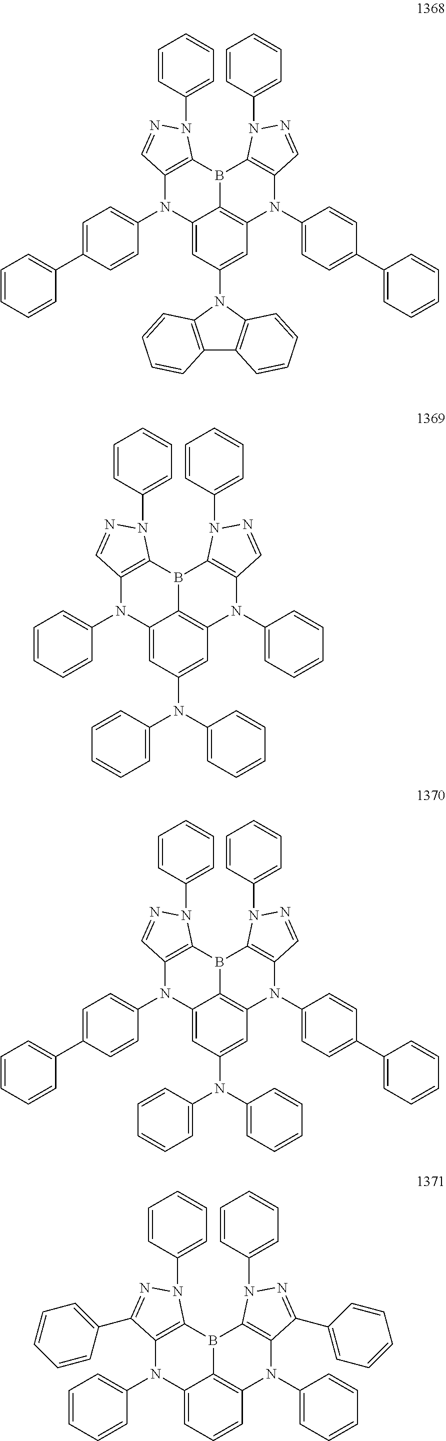

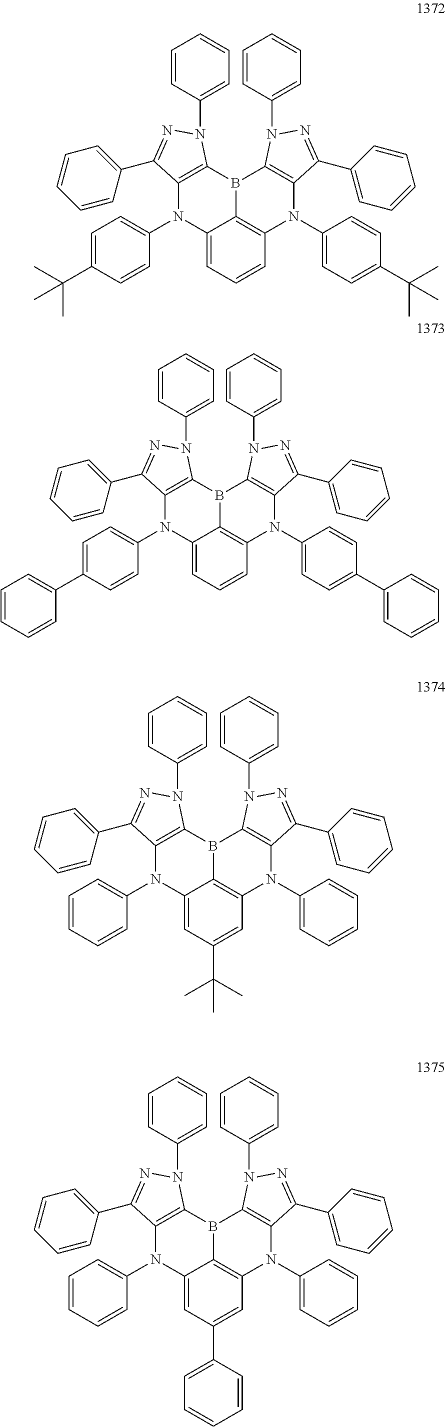 [SMILES: CC(C)(C)c1ccc(N2c3cccc4c3B(c3c2c(-c2ccccc2)nn3-c2ccccc2)c2c(c(-c3ccccc3)nn2-c2ccccc2)N4c2ccc(C(C)(C)C)cc2)cc1 ; c1ccc(-c2ccc(N3c4cccc5c4B(c4c3c(-c3ccccc3)nn4-c3ccccc3)c3c(c(-c4ccccc4)nn3-c3ccccc3)N5c3ccc(-c4ccccc4)cc3)cc2)cc1 ; CC(C)(C)c1cc2c3c(c1)N(c1ccccc1)c1c(-c4ccccc4)nn(-c4ccccc4)c1B3c1c(c(-c3ccccc3)nn1-c1ccccc1)N2c1ccccc1 ; c1ccc(-c2cc3c4c(c2)N(c2ccccc2)c2c(-c5ccccc5)nn(-c5ccccc5)c2B4c2c(c(-c4ccccc4)nn2-c2ccccc2)N3c2ccccc2)cc1]

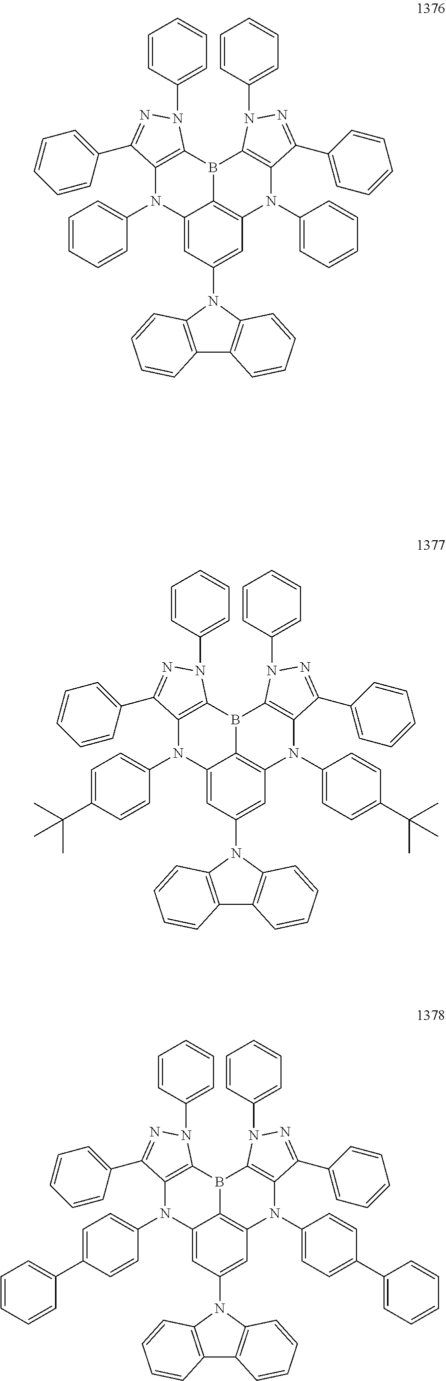 [SMILES: c1ccc(-c2nn(-c3ccccc3)c3c2N(c2ccccc2)c2cc(-n4c5ccccc5c5ccccc54)cc4c2B3c2c(c(-c3ccccc3)nn2-c2ccccc2)N4c2ccccc2)cc1 ; CC(C)(C)c1ccc(N2c3cc(-n4c5ccccc5c5ccccc54)cc4c3B(c3c2c(-c2ccccc2)nn3-c2ccccc2)c2c(c(-c3ccccc3)nn2-c2ccccc2)N4c2ccc(C(C)(C)C)cc2)cc1 ; c1ccc(-c2ccc(N3c4cc(-n5c6ccccc6c6ccccc65)cc5c4B(c4c3c(-c3ccccc3)nn4-c3ccccc3)c3c(c(-c4ccccc4)nn3-c3ccccc3)N5c3ccc(-c4ccccc4)cc3)cc2)cc1]

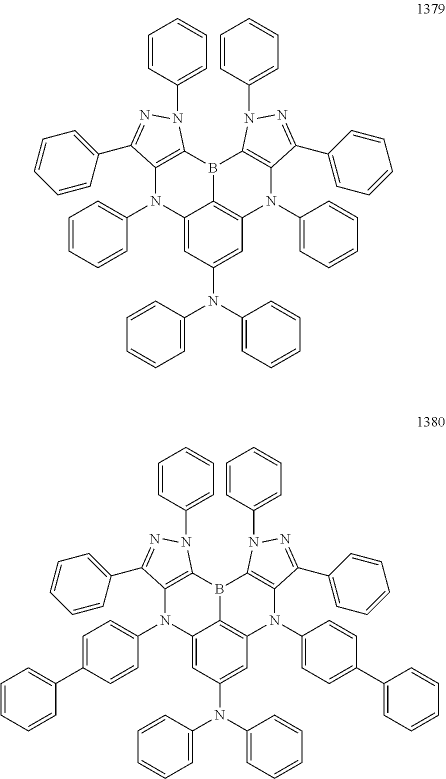 [SMILES: c1ccc(-c2nn(-c3ccccc3)c3c2N(c2ccccc2)c2cc(N(c4ccccc4)c4ccccc4)cc4c2B3c2c(c(-c3ccccc3)nn2-c2ccccc2)N4c2ccccc2)cc1 ; c1ccc(-c2ccc(N3c4cc(N(c5ccccc5)c5ccccc5)cc5c4B(c4c3c(-c3ccccc3)nn4-c3ccccc3)c3c(c(-c4ccccc4)nn3-c3ccccc3)N5c3ccc(-c4ccccc4)cc3)cc2)cc1]

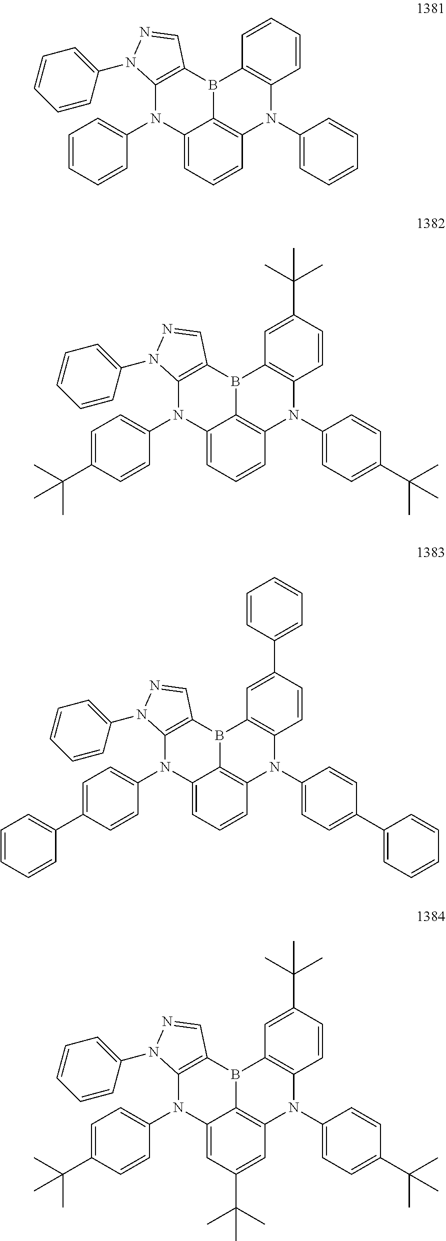

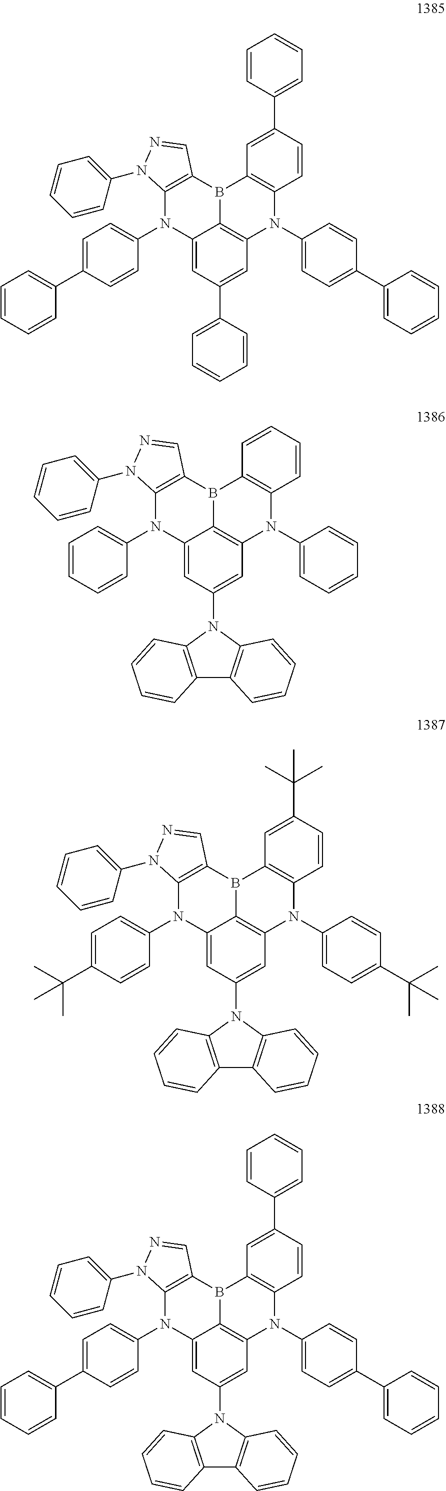

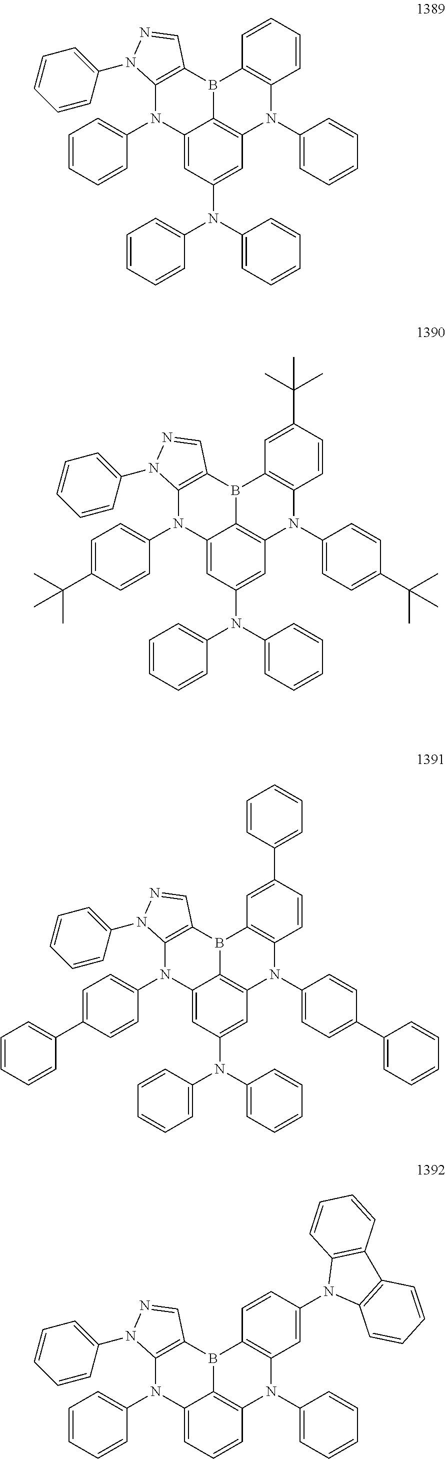 [SMILES: c1ccc(N(c2ccccc2)c2cc3c4c(c2)N(c2ccccc2)c2c(cnn2-c2ccccc2)B4c2ccccc2N3c2ccccc2)cc1 ; CC(C)(C)c1ccc(N2c3ccc(C(C)(C)C)cc3B3c4cnn(-c5ccccc5)c4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5cnn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4cnn(-c5ccccc5)c4N(c4ccccc4)c4cccc2c43)cc1]

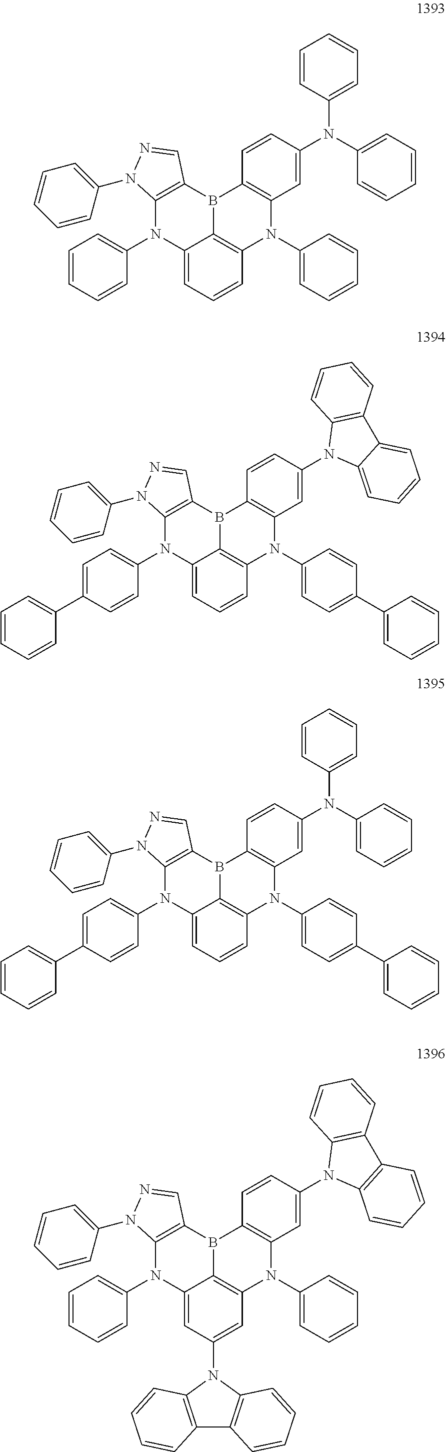 [SMILES: c1ccc(N(c2ccccc2)c2ccc3c(c2)N(c2ccccc2)c2cccc4c2B3c2cnn(-c3ccccc3)c2N4c2ccccc2)cc1 ; c1ccc(-c2ccc(N3c4cc(-n5c6ccccc6c6ccccc65)ccc4B4c5cnn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5cnn(-c6ccccc6)c5N(c5ccc(-c6ccccc6)cc5)c5cccc3c54)cc2)cc1 ; c1ccc(N2c3cc(-n4c5ccccc5c5ccccc54)ccc3B3c4cnn(-c5ccccc5)c4N(c4ccccc4)c4cc(-n5c6ccccc6c6ccccc65)cc2c43)cc1]

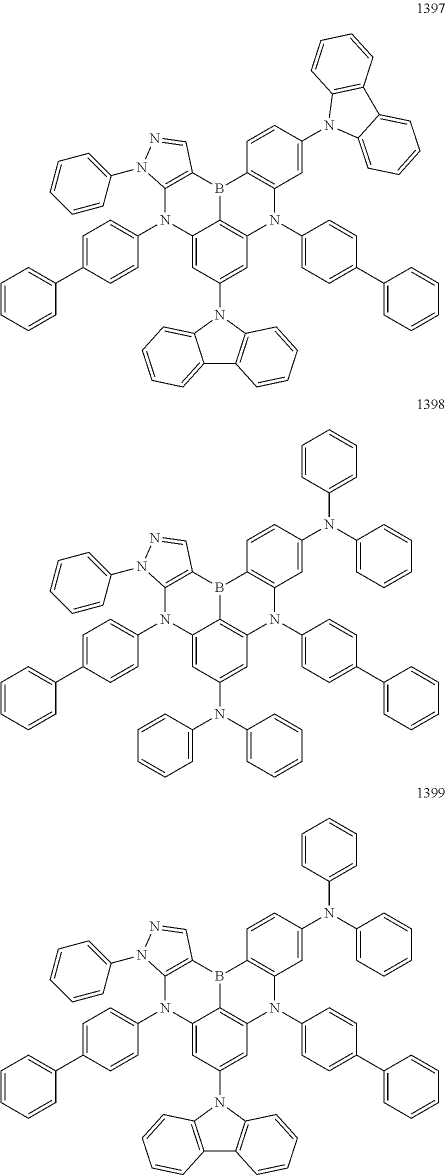 [SMILES: c1ccc(-c2ccc(N3c4cc(-n5c6ccccc6c6ccccc65)ccc4B4c5cnn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cc(N(c5ccccc5)c5ccccc5)ccc4B4c5cnn(-c6ccccc6)c5N(c5ccc(-c6ccccc6)cc5)c5cc(N(c6ccccc6)c6ccccc6)cc3c54)cc2)cc1 ; c1ccc(-c2ccc(N3c4cc(N(c5ccccc5)c5ccccc5)ccc4B4c5cnn(-c6ccccc6)c5N(c5ccc(-c6ccccc6)cc5)c5cc(-n6c7ccccc7c7ccccc76)cc3c54)cc2)cc1]

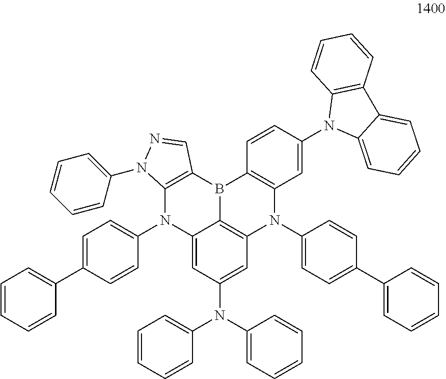 [SMILES: c1ccc(-c2ccc(N3c4cc(-n5c6ccccc6c6ccccc65)ccc4B4c5cnn(-c6ccccc6)c5N(c5ccc(-c6ccccc6)cc5)c5cc(N(c6ccccc6)c6ccccc6)cc3c54)cc2)cc1]

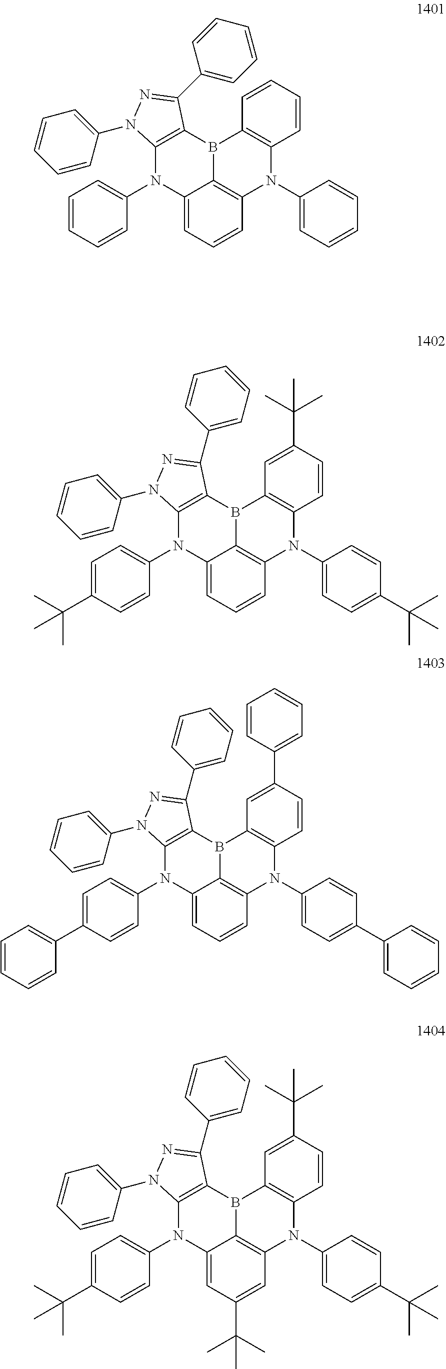

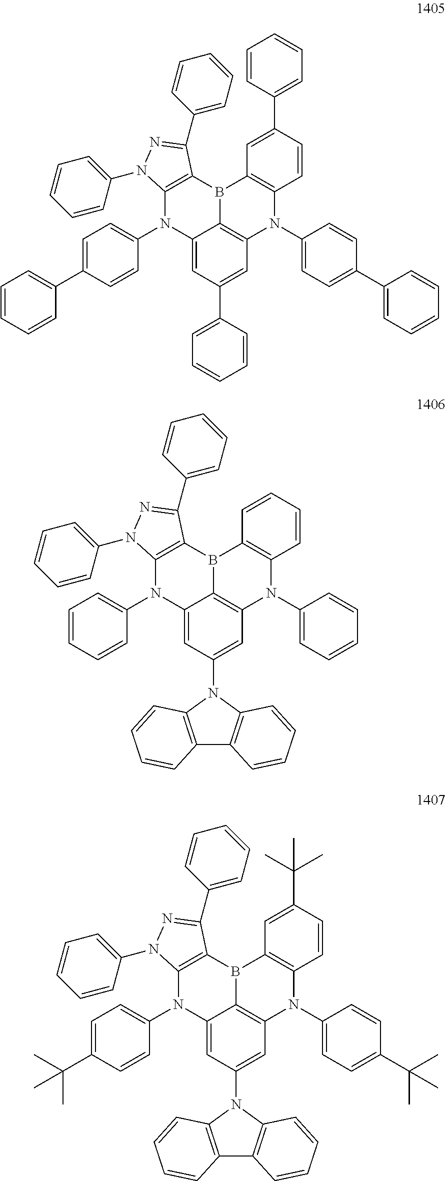

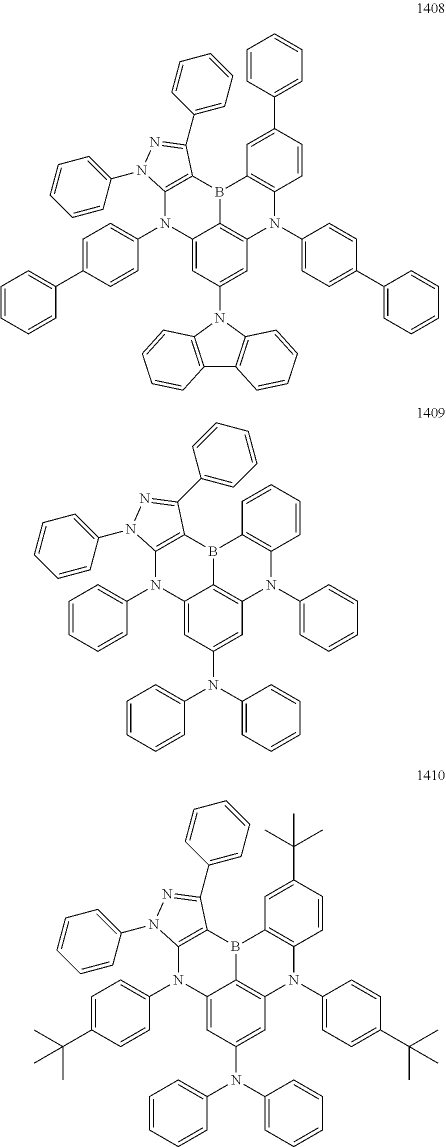

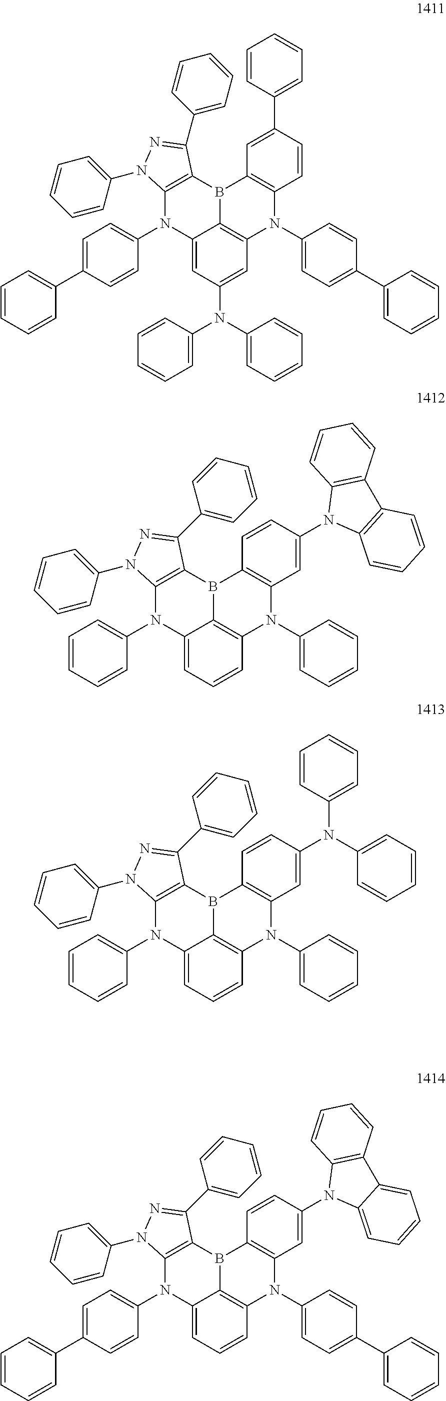 [SMILES: c1ccc(-c2ccc(N3c4ccc(-c5ccccc5)cc4B4c5c3cc(N(c3ccccc3)c3ccccc3)cc5N(c3ccc(-c5ccccc5)cc3)c3c4c(-c4ccccc4)nn3-c3ccccc3)cc2)cc1 ; c1ccc(-c2nn(-c3ccccc3)c3c2B2c4ccc(-n5c6ccccc6c6ccccc65)cc4N(c4ccccc4)c4cccc(c42)N3c2ccccc2)cc1 ; c1ccc(-c2nn(-c3ccccc3)c3c2B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5c3cccc5N(c3ccc(-c5ccccc5)cc3)c3c4c(-c4ccccc4)nn3-c3ccccc3)cc2)cc1]

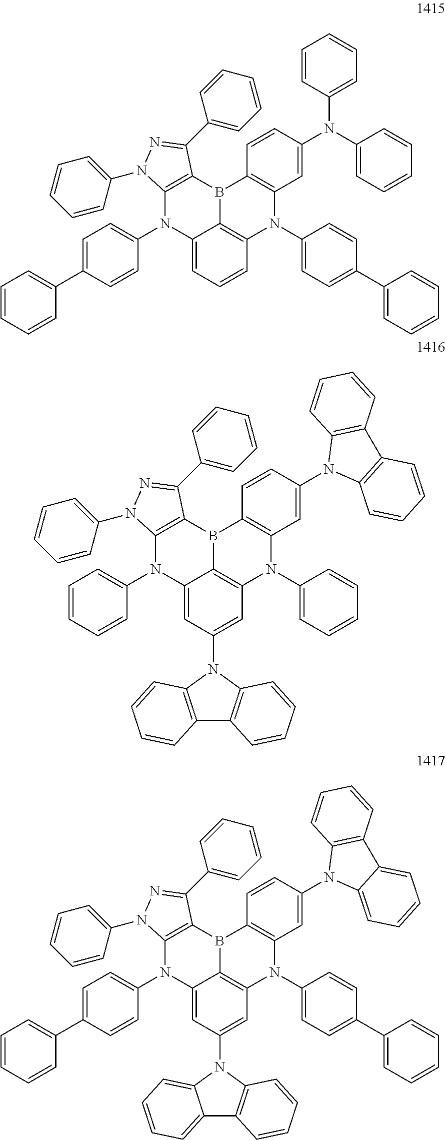 [SMILES: c1ccc(-c2ccc(N3c4cc(N(c5ccccc5)c5ccccc5)ccc4B4c5c3cccc5N(c3ccc(-c5ccccc5)cc3)c3c4c(-c4ccccc4)nn3-c3ccccc3)cc2)cc1 ; c1ccc(-c2nn(-c3ccccc3)c3c2B2c4ccc(-n5c6ccccc6c6ccccc65)cc4N(c4ccccc4)c4cc(-n5c6ccccc6c6ccccc65)cc(c42)N3c2ccccc2)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3c4c(-c4ccccc4)nn3-c3ccccc3)cc2)cc1]

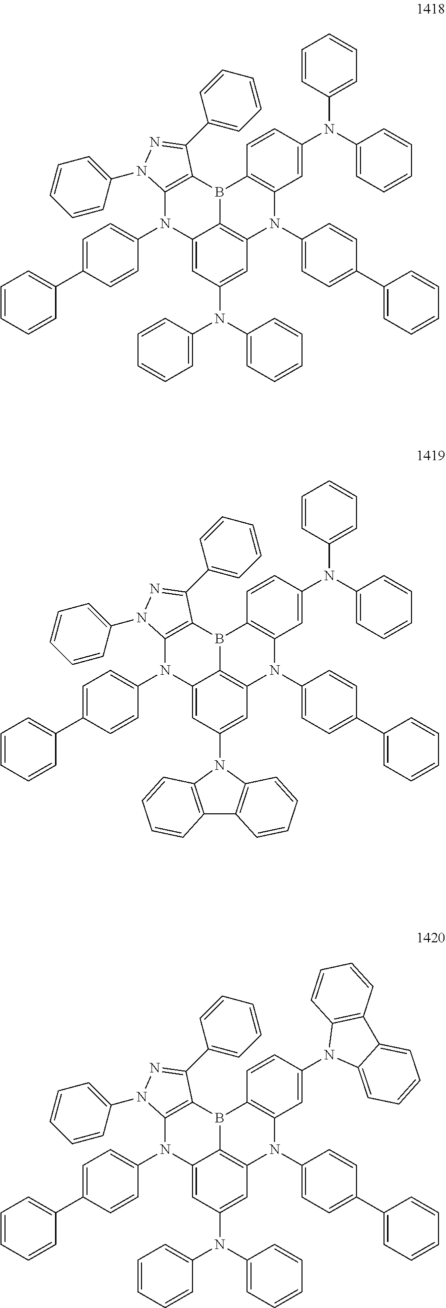 [SMILES: c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3c4c(-c4ccccc4)nn3-c3ccccc3)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3c4c(-c4ccccc4)nn3-c3ccccc3)cc2)cc1]

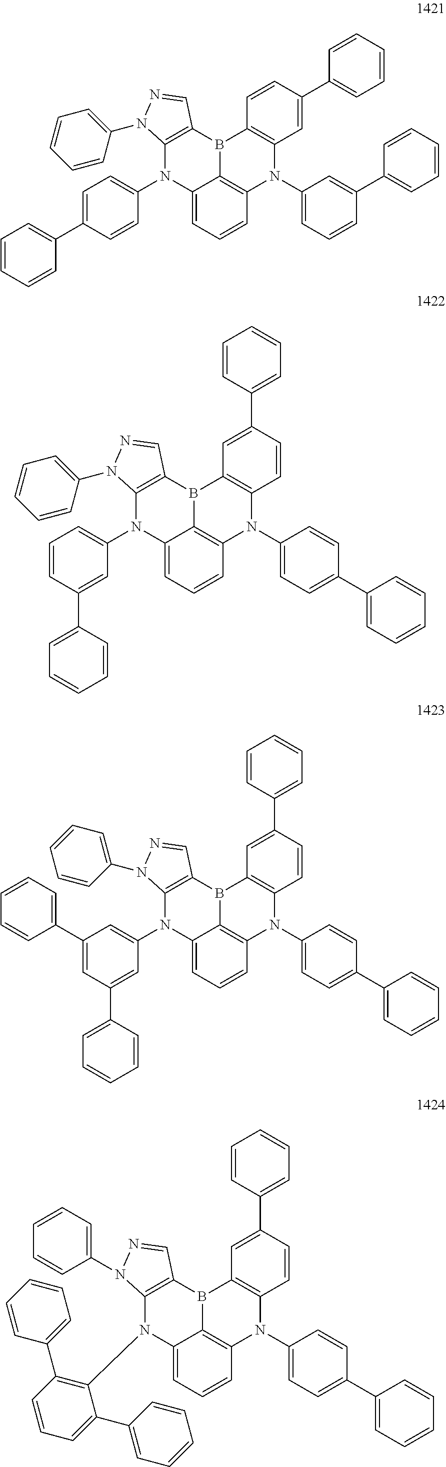 [SMILES: c1ccc(-c2ccc(N3c4cccc5c4B(c4ccc(-c6ccccc6)cc4N5c4cccc(-c5ccccc5)c4)c4cnn(-c5ccccc5)c43)cc2)cc1 ; c1ccc(-c2ccc(N3c4ccc(-c5ccccc5)cc4B4c5cnn(-c6ccccc6)c5N(c5cccc(-c6ccccc6)c5)c5cccc3c54)cc2)cc1 ; c1ccc(-c2ccc(N3c4ccc(-c5ccccc5)cc4B4c5cnn(-c6ccccc6)c5N(c5cc(-c6ccccc6)cc(-c6ccccc6)c5)c5cccc3c54)cc2)cc1 ; c1ccc(-c2ccc(N3c4ccc(-c5ccccc5)cc4B4c5cnn(-c6ccccc6)c5N(c5c(-c6ccccc6)cccc5-c5ccccc5)c5cccc3c54)cc2)cc1]

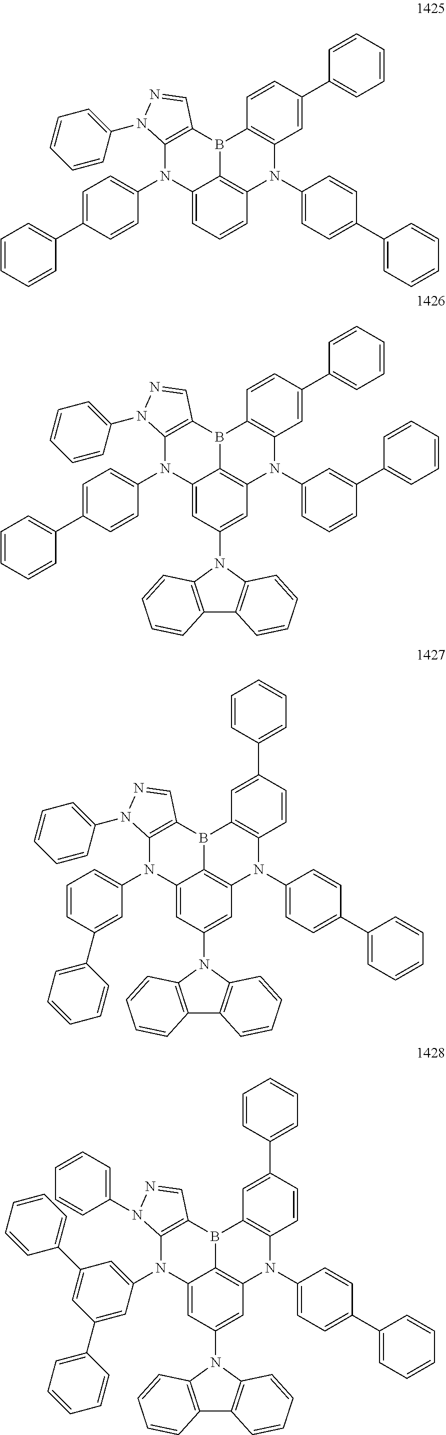 [SMILES: c1ccc(-c2ccc(N3c4cc(-c5ccccc5)ccc4B4c5cnn(-c6ccccc6)c5N(c5ccc(-c6ccccc6)cc5)c5cccc3c54)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4cnn(-c5ccccc5)c43)cc2)cc1 ; c1ccc(-c2ccc(N3c4ccc(-c5ccccc5)cc4B4c5cnn(-c6ccccc6)c5N(c5cccc(-c6ccccc6)c5)c5cc(-n6c7ccccc7c7ccccc76)cc3c54)cc2)cc1 ; c1ccc(-c2ccc(N3c4ccc(-c5ccccc5)cc4B4c5cnn(-c6ccccc6)c5N(c5cc(-c6ccccc6)cc(-c6ccccc6)c5)c5cc(-n6c7ccccc7c7ccccc76)cc3c54)cc2)cc1]

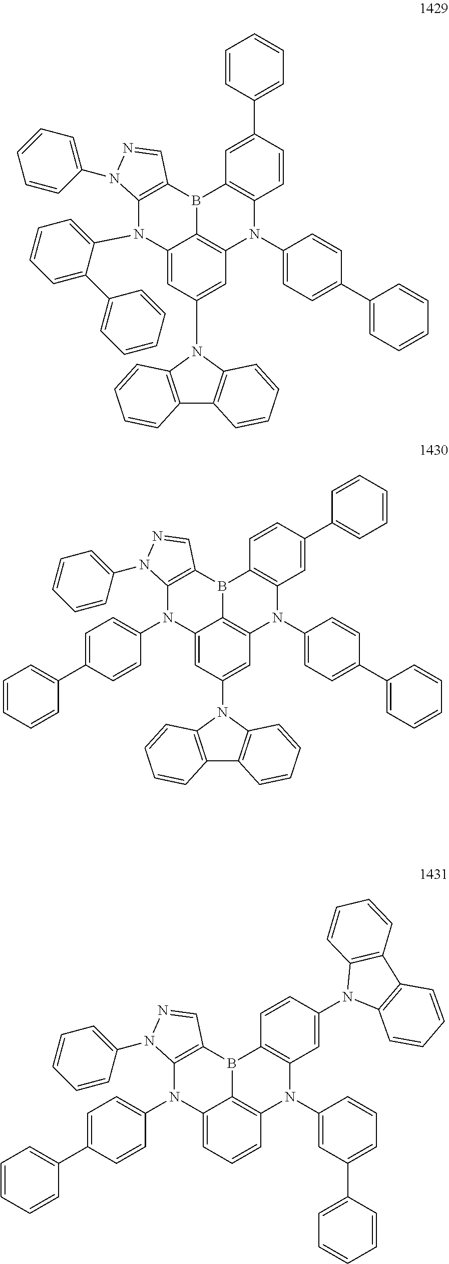 [SMILES: c1ccc(-c2ccc(N3c4ccc(-c5ccccc5)cc4B4c5cnn(-c6ccccc6)c5N(c5ccccc5-c5ccccc5)c5cc(-n6c7ccccc7c7ccccc76)cc3c54)cc2)cc1 ; c1ccc(-c2ccc(N3c4cc(-c5ccccc5)ccc4B4c5cnn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cccc5c4B(c4ccc(-n6c7ccccc7c7ccccc76)cc4N5c4cccc(-c5ccccc5)c4)c4cnn(-c5ccccc5)c43)cc2)cc1]

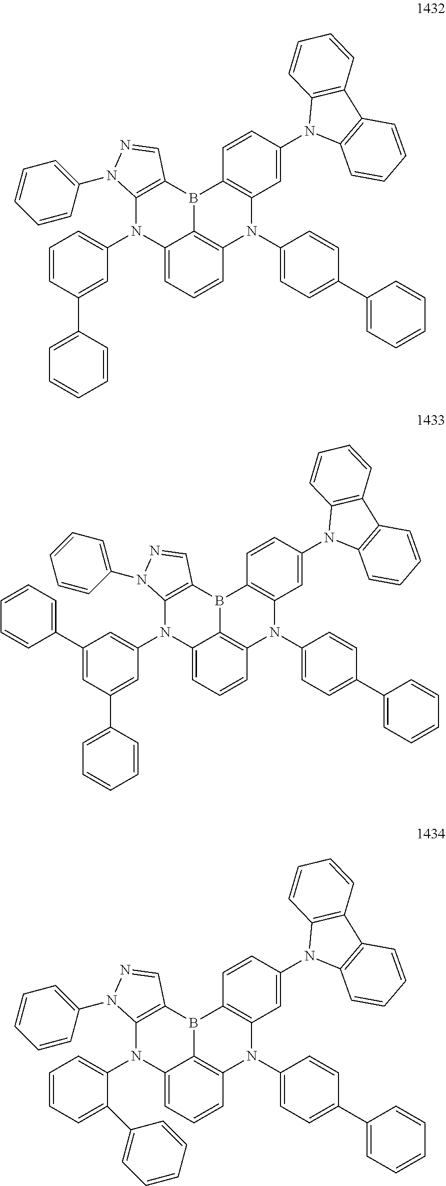 [SMILES: c1ccc(-c2ccc(N3c4cc(-n5c6ccccc6c6ccccc65)ccc4B4c5cnn(-c6ccccc6)c5N(c5cccc(-c6ccccc6)c5)c5cccc3c54)cc2)cc1 ; c1ccc(-c2ccc(N3c4cc(-n5c6ccccc6c6ccccc65)ccc4B4c5cnn(-c6ccccc6)c5N(c5cc(-c6ccccc6)cc(-c6ccccc6)c5)c5cccc3c54)cc2)cc1 ; c1ccc(-c2ccc(N3c4cc(-n5c6ccccc6c6ccccc65)ccc4B4c5cnn(-c6ccccc6)c5N(c5ccccc5-c5ccccc5)c5cccc3c54)cc2)cc1]

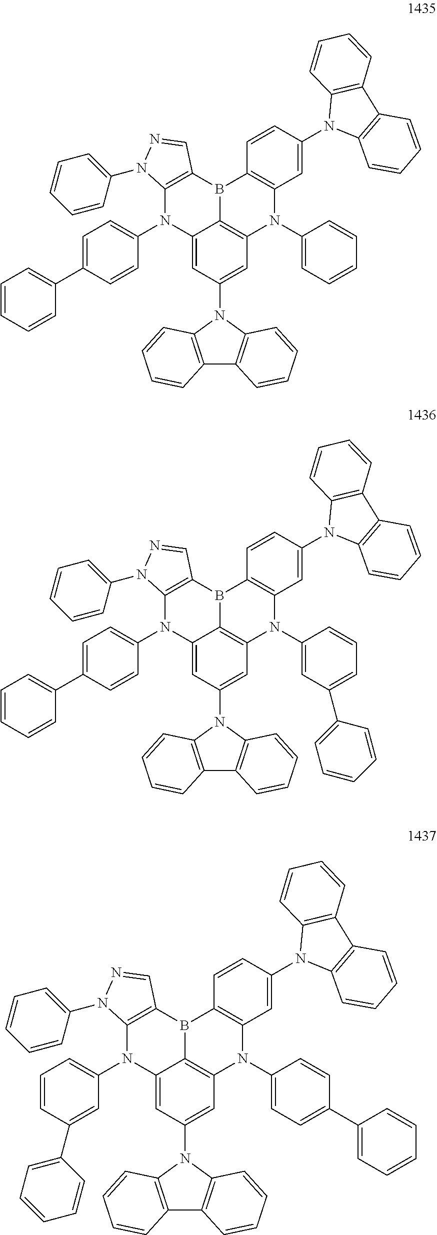 [SMILES: c1ccc(-c2ccc(N3c4cc(-n5c6ccccc6c6ccccc65)cc5c4B(c4ccc(-n6c7ccccc7c7ccccc76)cc4N5c4ccccc4)c4cnn(-c5ccccc5)c43)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4cnn(-c5ccccc5)c43)cc2)cc1 ; c1ccc(-c2ccc(N3c4cc(-n5c6ccccc6c6ccccc65)ccc4B4c5cnn(-c6ccccc6)c5N(c5cccc(-c6ccccc6)c5)c5cc(-n6c7ccccc7c7ccccc76)cc3c54)cc2)cc1]

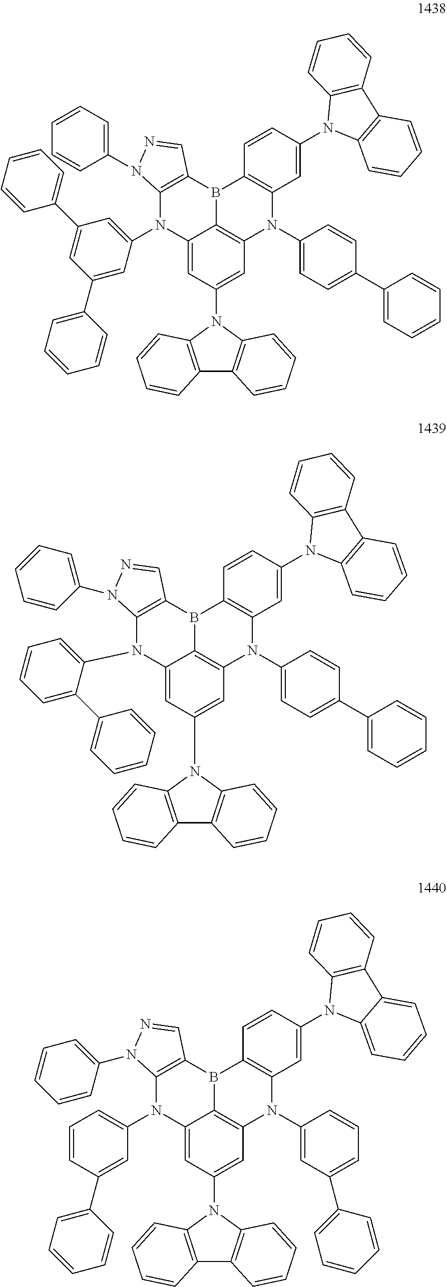 [SMILES: c1ccc(-c2ccc(N3c4cc(-n5c6ccccc6c6ccccc65)ccc4B4c5cnn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5cnn(-c6ccccc6)c5N(c5ccccc5-c5ccccc5)c5cc(-n6c7ccccc7c7ccccc76)cc3c54)cc2)cc1 ; c1ccc(-c2cccc(N3c4cc(-n5c6ccccc6c6ccccc65)ccc4B4c5cnn(-c6ccccc6)c5N(c5cccc(-c6ccccc6)c5)c5cc(-n6c7ccccc7c7ccccc76)cc3c54)c2)cc1]

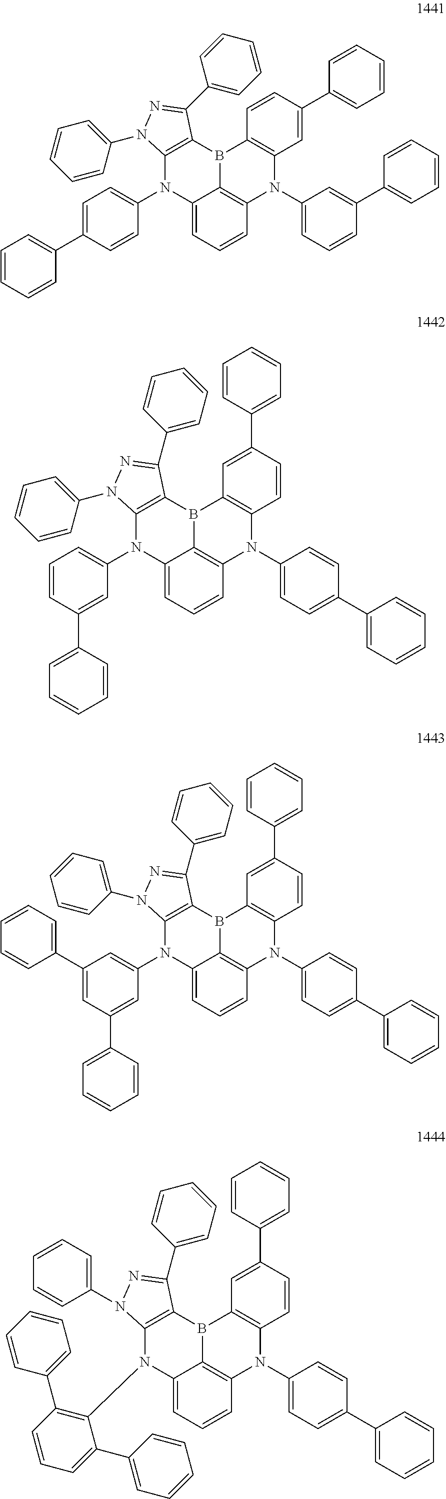 [SMILES: c1ccc(-c2ccc(N3c4cccc5c4B(c4ccc(-c6ccccc6)cc4N5c4cccc(-c5ccccc5)c4)c4c(-c5ccccc5)nn(-c5ccccc5)c43)cc2)cc1 ; c1ccc(-c2ccc(N3c4ccc(-c5ccccc5)cc4B4c5c3cccc5N(c3cccc(-c5ccccc5)c3)c3c4c(-c4ccccc4)nn3-c3ccccc3)cc2)cc1 ; c1ccc(-c2ccc(N3c4ccc(-c5ccccc5)cc4B4c5c3cccc5N(c3cc(-c5ccccc5)cc(-c5ccccc5)c3)c3c4c(-c4ccccc4)nn3-c3ccccc3)cc2)cc1 ; c1ccc(-c2ccc(N3c4ccc(-c5ccccc5)cc4B4c5c3cccc5N(c3c(-c5ccccc5)cccc3-c3ccccc3)c3c4c(-c4ccccc4)nn3-c3ccccc3)cc2)cc1]

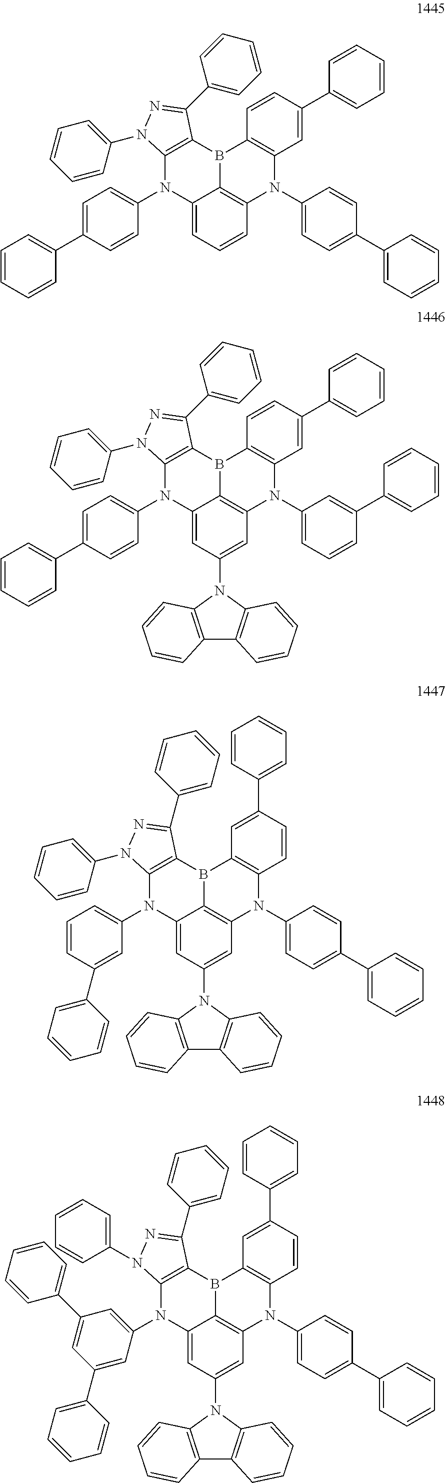 [SMILES: c1ccc(-c2ccc(N3c4cc(-c5ccccc5)ccc4B4c5c3cccc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c(-c5ccccc5)nn(-c5ccccc5)c43)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3c4c(-c4ccccc4)nn3-c3ccccc3)cc2)cc1 ; c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3c4c(-c4ccccc4)nn3-c3ccccc3)cc2)cc1]

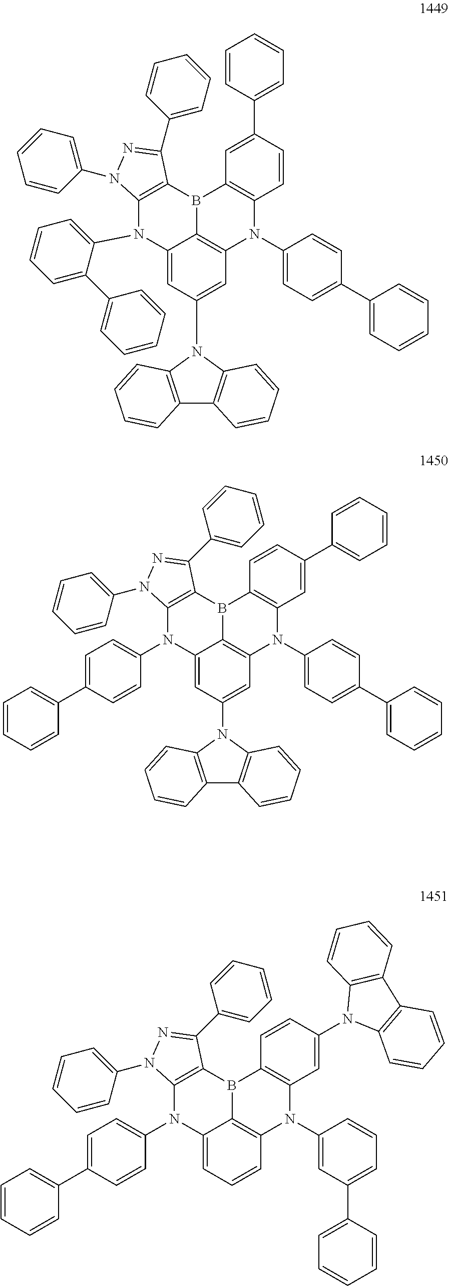 [SMILES: c1ccc(-c2ccc(N3c4ccc(-c5ccccc5)cc4B4c5c3cc(-n3c6ccccc6c6ccccc63)cc5N(c3ccccc3-c3ccccc3)c3c4c(-c4ccccc4)nn3-c3ccccc3)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3c4c(-c4ccccc4)nn3-c3ccccc3)cc2)cc1 ; c1ccc(-c2ccc(N3c4cccc5c4B(c4ccc(-n6c7ccccc7c7ccccc76)cc4N5c4cccc(-c5ccccc5)c4)c4c(-c5ccccc5)nn(-c5ccccc5)c43)cc2)cc1]

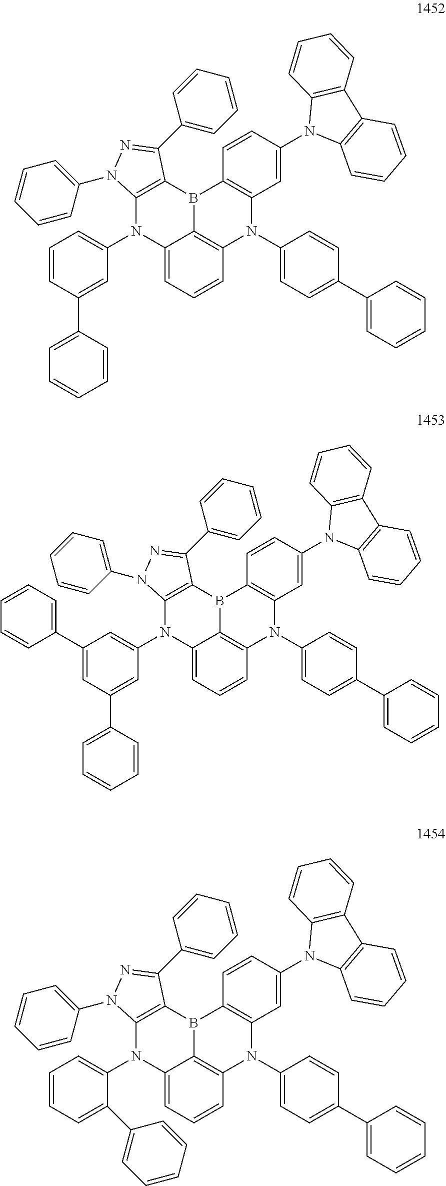 [SMILES: c1ccc(-c2ccc(N3c4cc(-n5c6ccccc6c6ccccc65)ccc4B4c5c3cccc5N(c3cccc(-c5ccccc5)c3)c3c4c(-c4ccccc4)nn3-c3ccccc3)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3c4c(-c4ccccc4)nn3-c3ccccc3)cc2)cc1 ; c1ccc(-c2ccc(N3c4cc(-n5c6ccccc6c6ccccc65)ccc4B4c5c3cccc5N(c3ccccc3-c3ccccc3)c3c4c(-c4ccccc4)nn3-c3ccccc3)cc2)cc1]

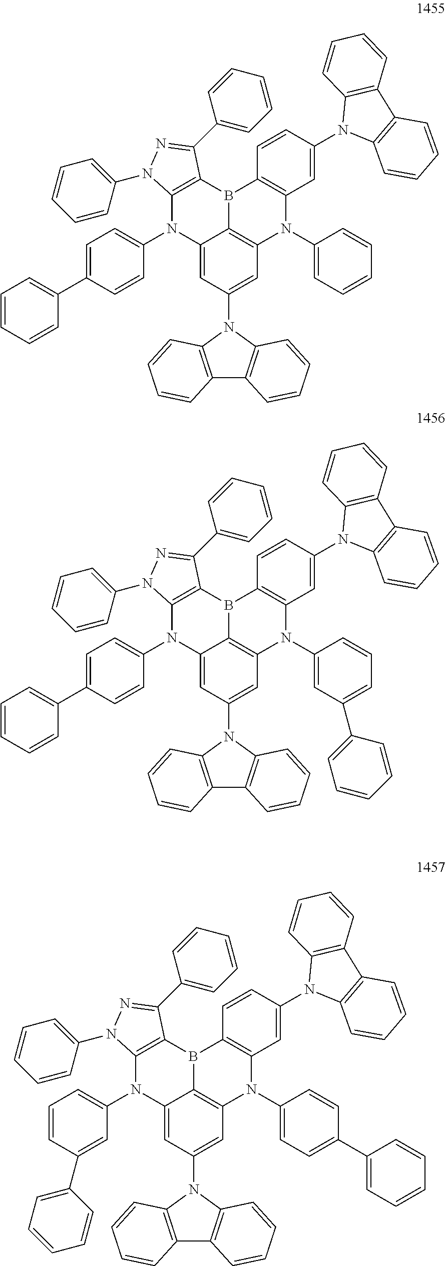 [SMILES: c1ccc(-c2ccc(N3c4cc(-n5c6ccccc6c6ccccc65)cc5c4B(c4ccc(-n6c7ccccc7c7ccccc76)cc4N5c4ccccc4)c4c(-c5ccccc5)nn(-c5ccccc5)c43)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c(-c5ccccc5)nn(-c5ccccc5)c43)cc2)cc1 ; c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3c4c(-c4ccccc4)nn3-c3ccccc3)cc2)cc1]

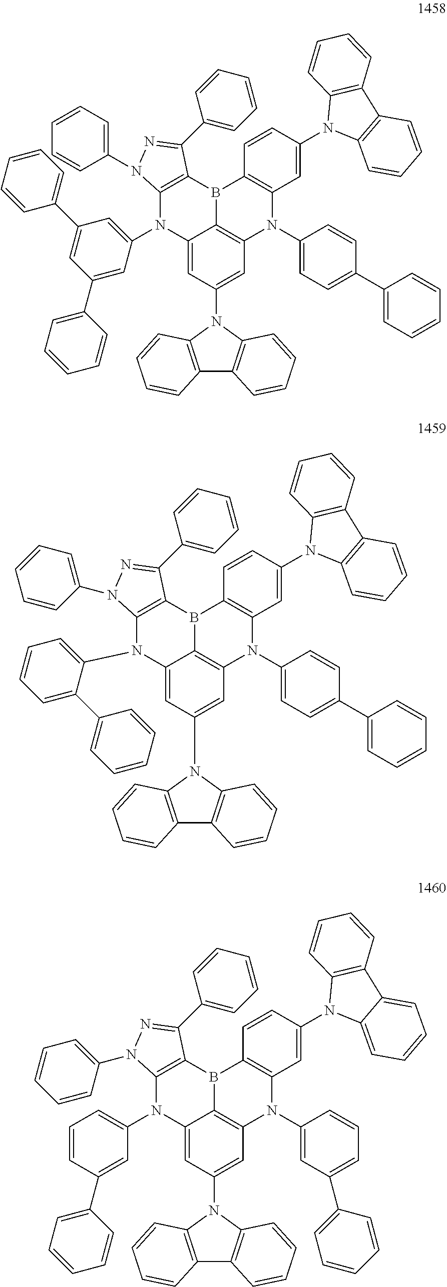 [SMILES: c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3c4c(-c4ccccc4)nn3-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3c4c(-c4ccccc4)nn3-c3ccccc3)cc2)cc1 ; c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3c4c(-c4ccccc4)nn3-c3ccccc3)c2)cc1]

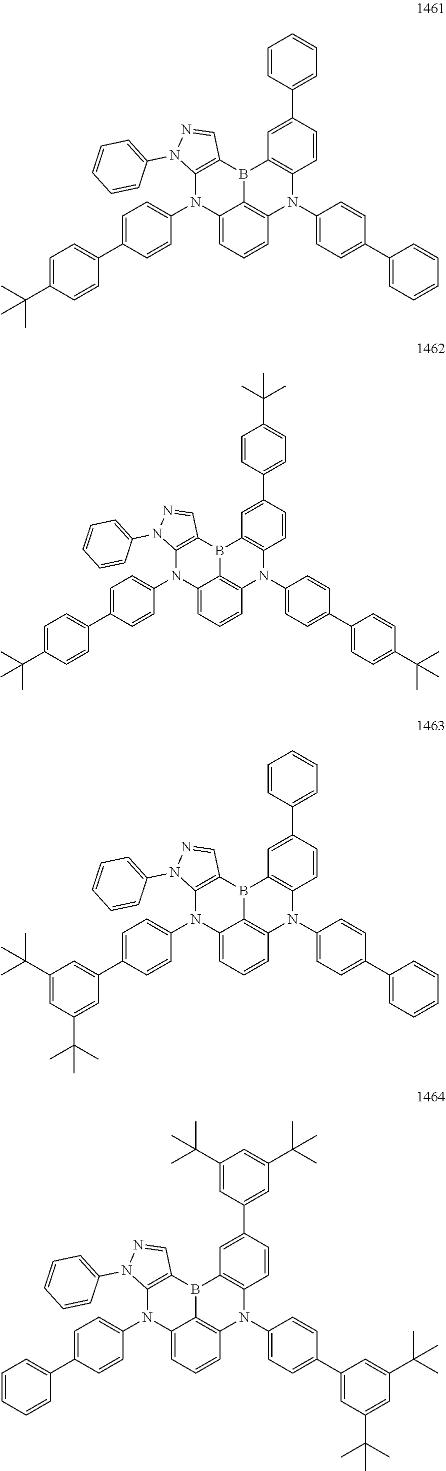

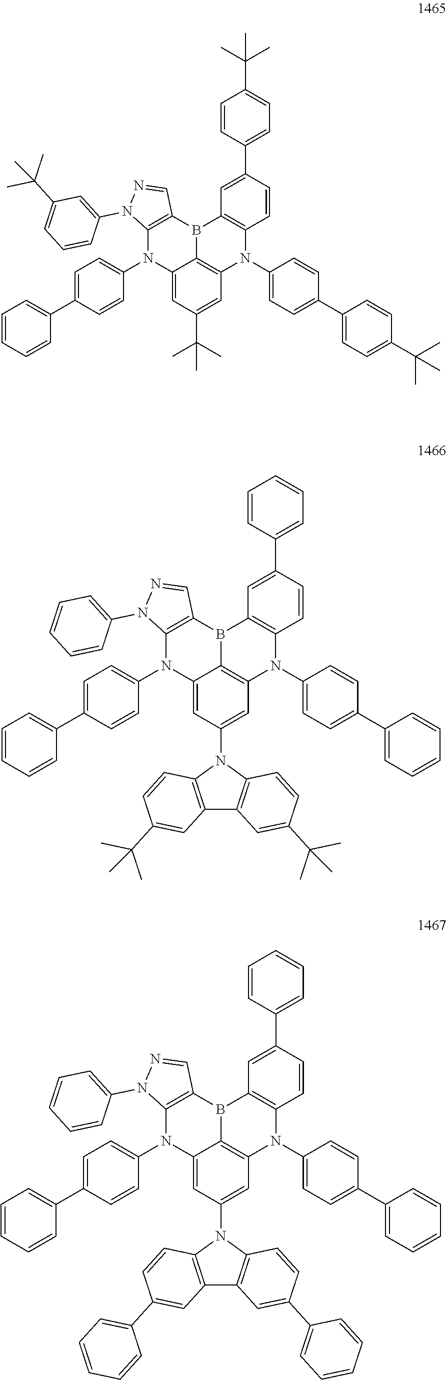 [SMILES: CC(C)(C)c1ccc(-c2ccc(N3c4ccc(-c5ccc(C(C)(C)C)cc5)cc4B4c5cnn(-c6cccc(C(C)(C)C)c6)c5N(c5ccc(-c6ccccc6)cc5)c5cc(C(C)(C)C)cc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(cnn1-c1ccccc1)B3c1cc(-c3ccccc3)ccc1N2c1ccc(-c2ccccc2)cc1 ; c1ccc(-c2ccc(N3c4ccc(-c5ccccc5)cc4B4c5cnn(-c6ccccc6)c5N(c5ccc(-c6ccccc6)cc5)c5cc(-n6c7ccc(-c8ccccc8)cc7c7cc(-c8ccccc8)ccc76)cc3c54)cc2)cc1]

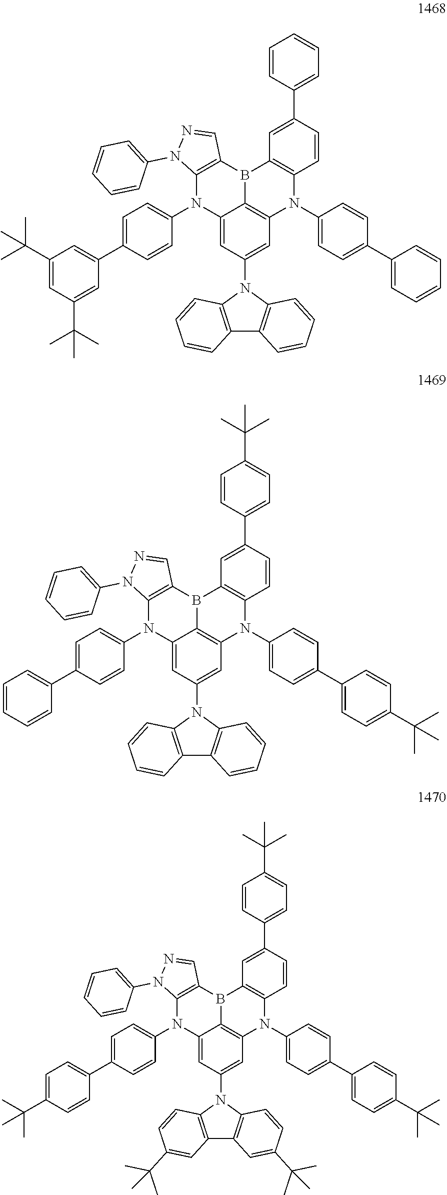

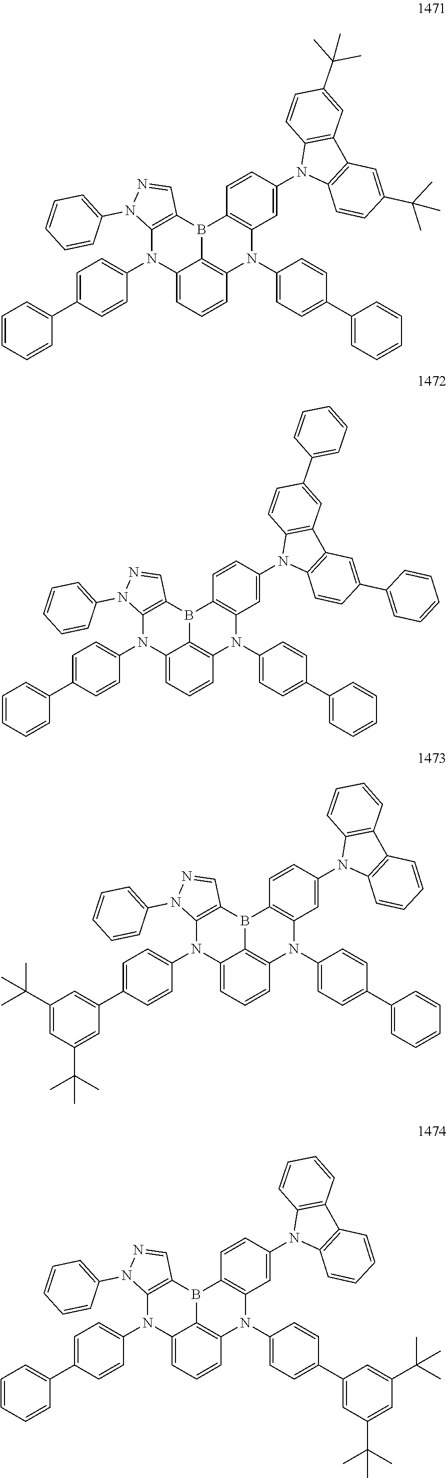

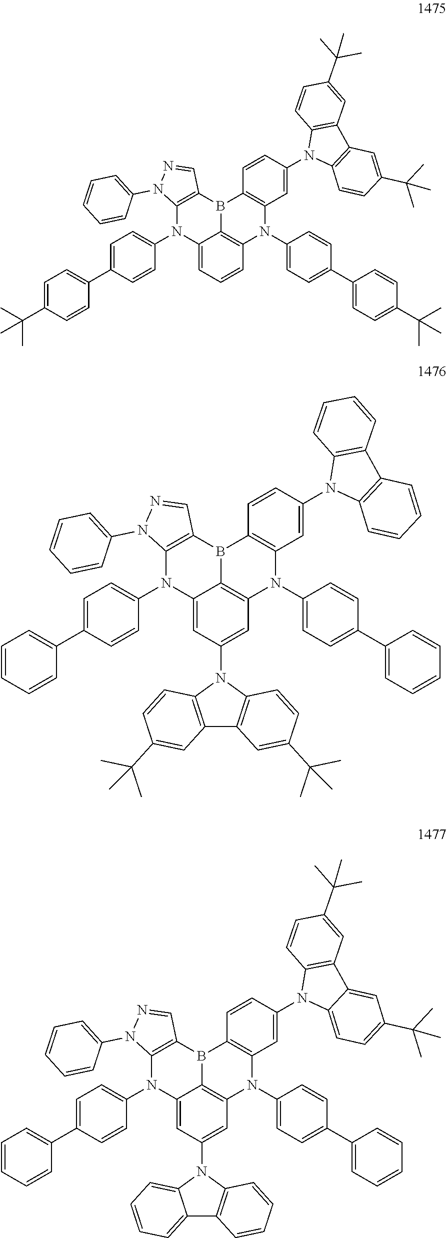 [SMILES: CC(C)(C)c1ccc(-c2ccc(N3c4cc(-n5c6ccc(C(C)(C)C)cc6c6cc(C(C)(C)C)ccc65)ccc4B4c5cnn(-c6ccccc6)c5N(c5ccc(-c6ccc(C(C)(C)C)cc6)cc5)c5cccc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(cnn1-c1ccccc1)B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1cnn(-c2ccccc2)c1N3c1ccc(-c2ccccc2)cc1]

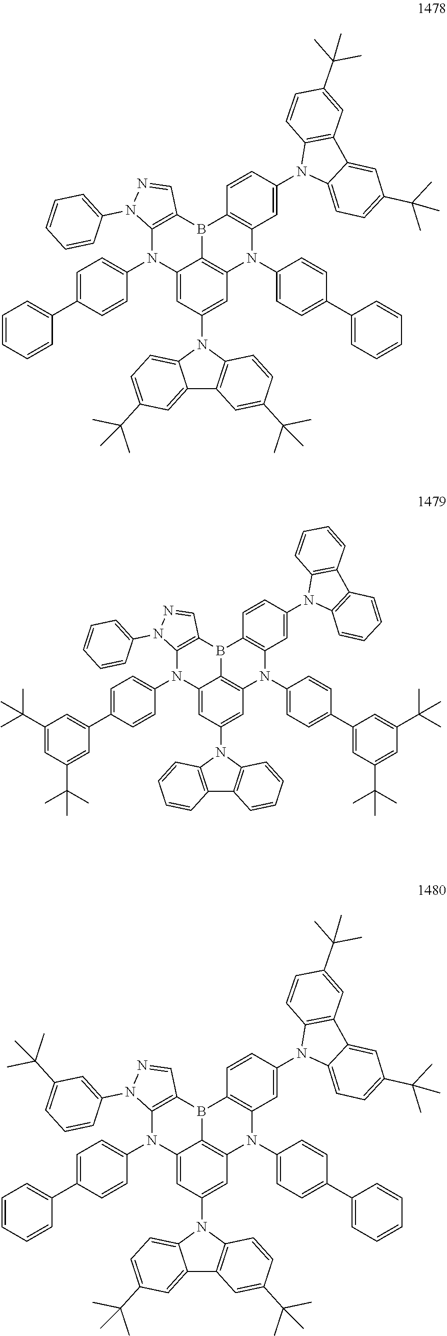

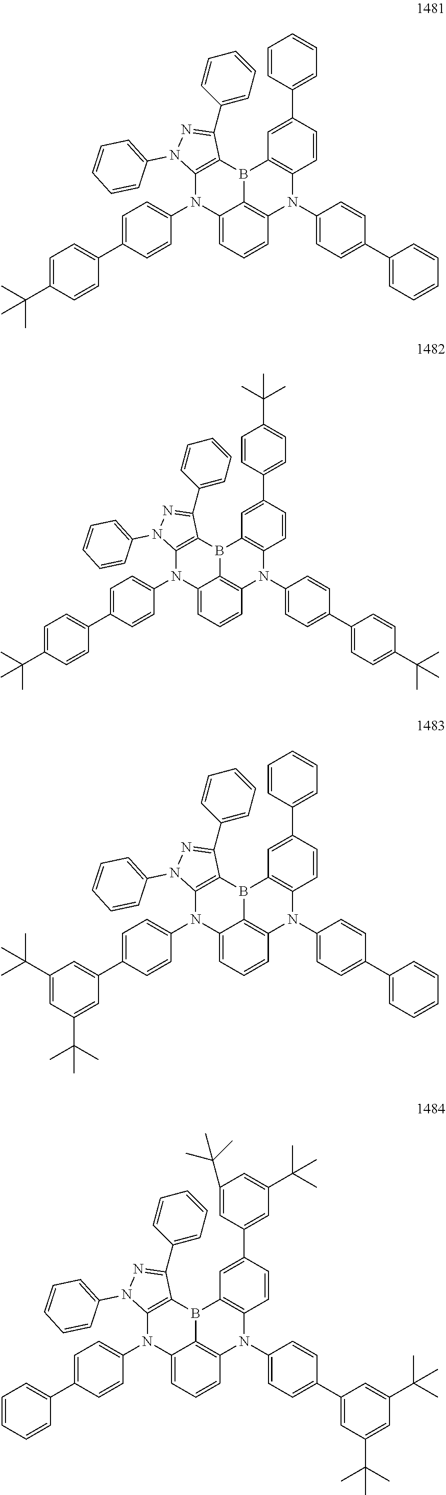

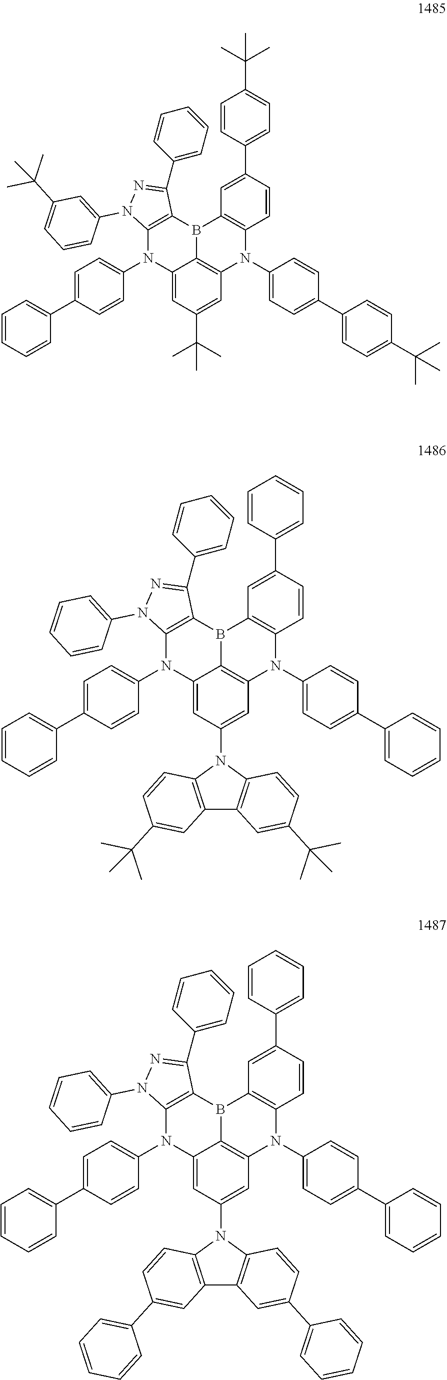 [SMILES: CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3c4c(-c4ccccc4)nn3-c3cccc(C(C)(C)C)c3)cc2)cc1 ; CC(C)(C)c1ccc2c(c1)c1cc(C(C)(C)C)ccc1n2-c1cc2c3c(c1)N(c1ccc(-c4ccccc4)cc1)c1c(c(-c4ccccc4)nn1-c1ccccc1)B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3c4c(-c4ccccc4)nn3-c3ccccc3)cc2)cc1]

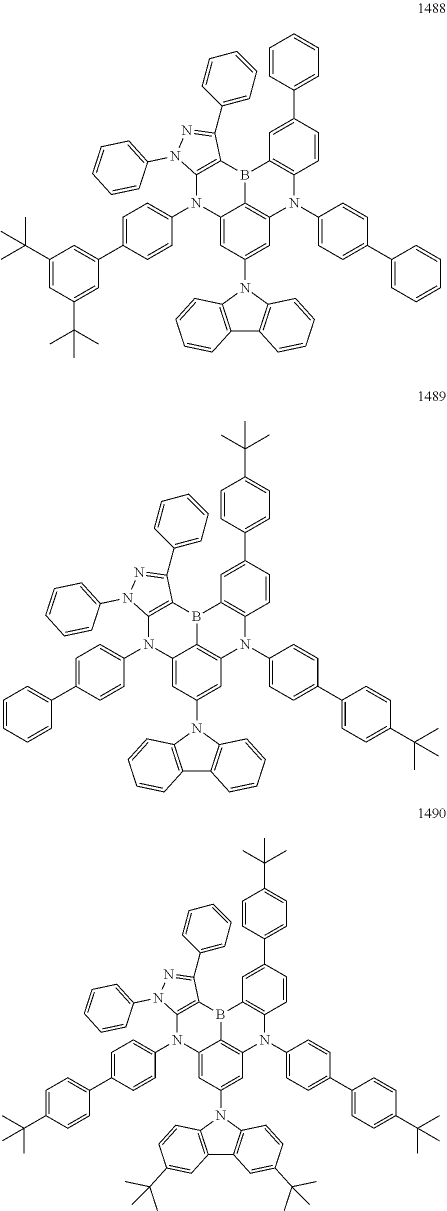

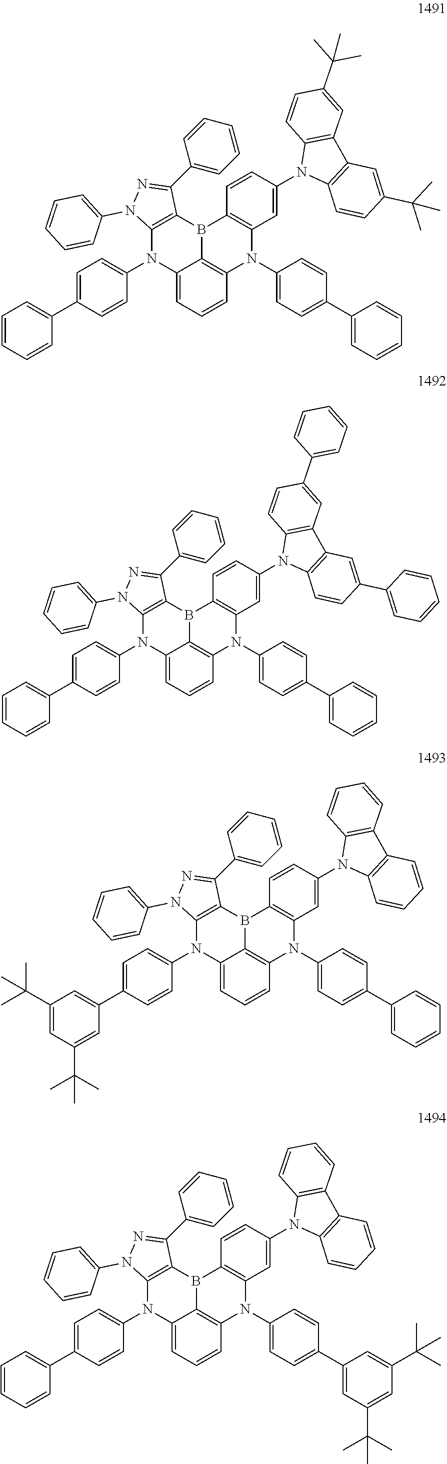

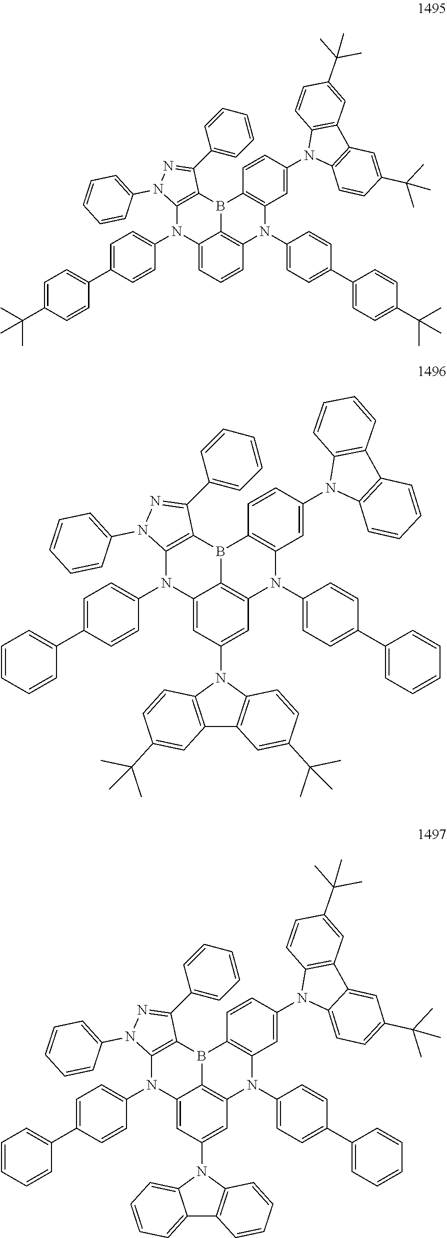 [SMILES: CC(C)(C)c1ccc(-c2ccc(N3c4cc(-n5c6ccc(C(C)(C)C)cc6c6cc(C(C)(C)C)ccc65)ccc4B4c5c3cccc5N(c3ccc(-c5ccc(C(C)(C)C)cc5)cc3)c3c4c(-c4ccccc4)nn3-c3ccccc3)cc2)cc1 ; CC(C)(C)c1ccc2c(c1)c1cc(C(C)(C)C)ccc1n2-c1cc2c3c(c1)N(c1ccc(-c4ccccc4)cc1)c1c(c(-c4ccccc4)nn1-c1ccccc1)B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(-c2ccccc2)nn(-c2ccccc2)c1N3c1ccc(-c2ccccc2)cc1]

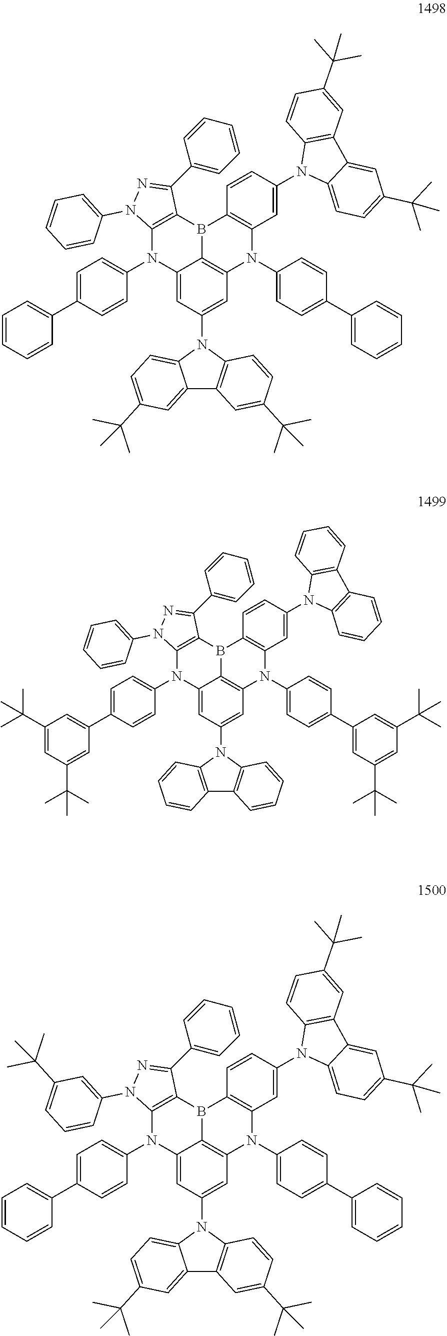

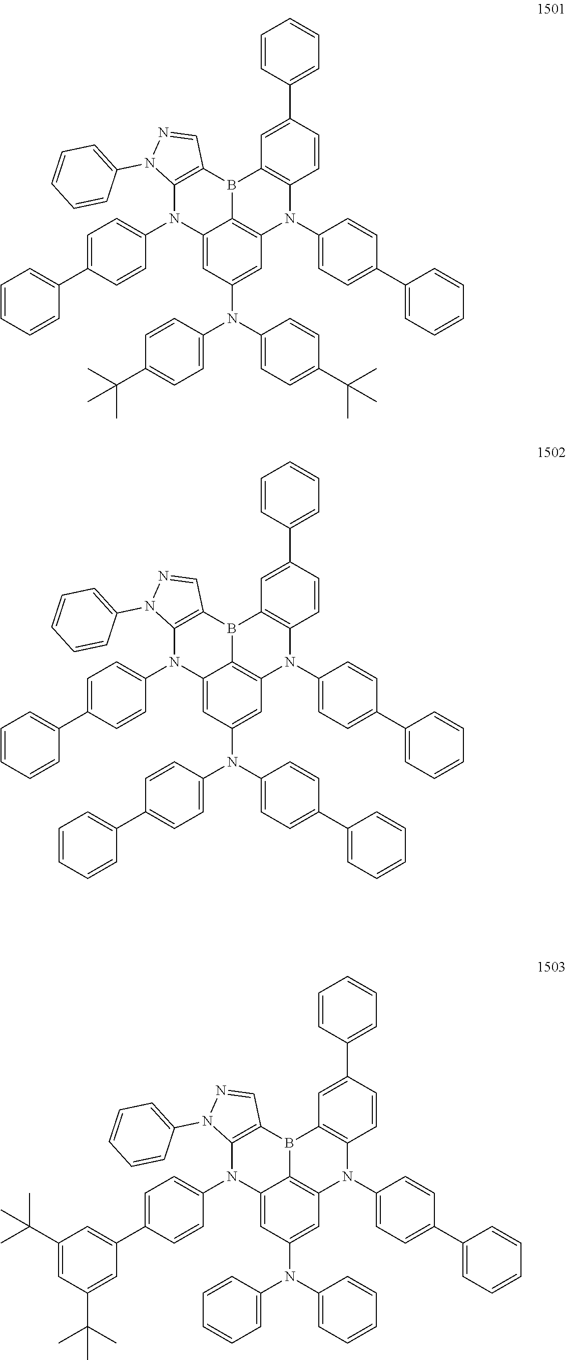 [SMILES: CC(C)(C)c1ccc(N(c2ccc(C(C)(C)C)cc2)c2cc3c4c(c2)N(c2ccc(-c5ccccc5)cc2)c2c(cnn2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(cnn3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4cnn(-c5ccccc5)c43)cc2)cc(C(C)(C)C)c1]

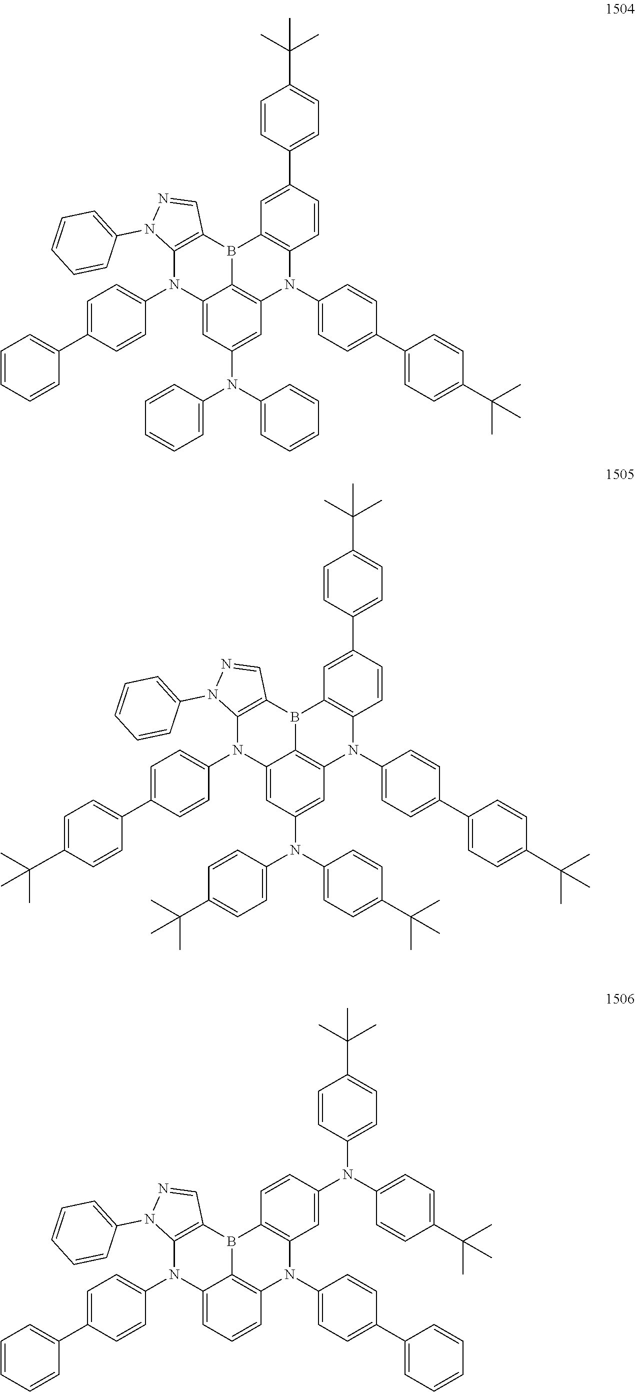

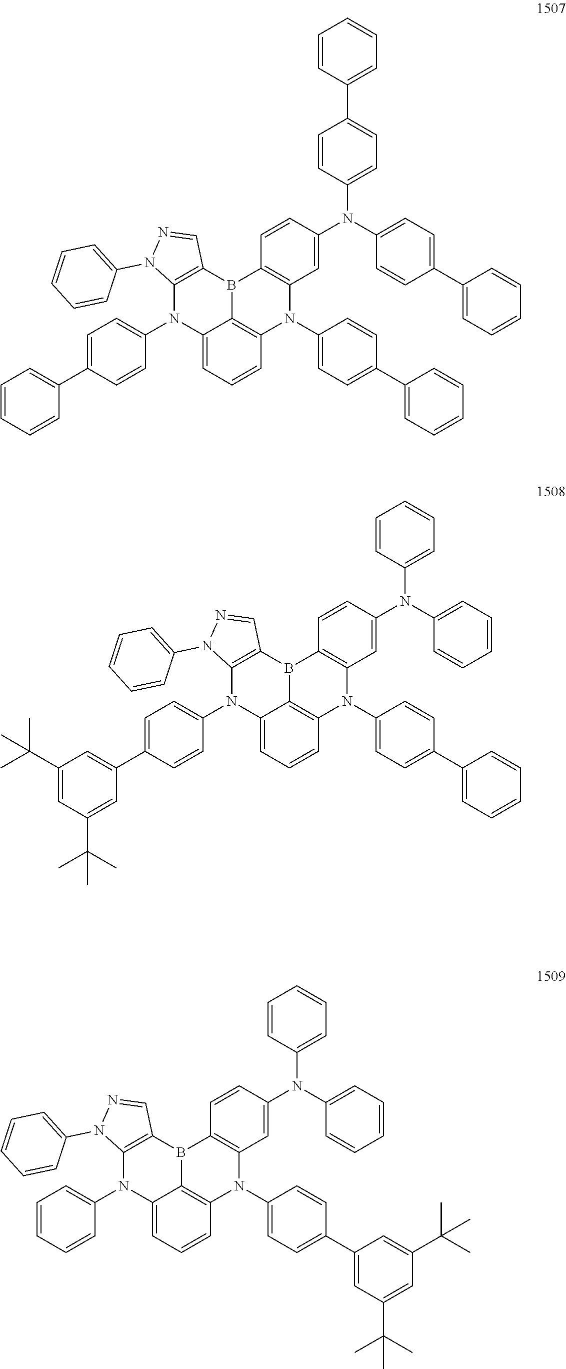 [SMILES: c1ccc(-c2ccc(N(c3ccc(-c4ccccc4)cc3)c3ccc4c(c3)N(c3ccc(-c5ccccc5)cc3)c3cccc5c3B4c3cnn(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4cnn(-c5ccccc5)c43)cc2)cc(C(C)(C)C)c1 ; CC(C)(C)c1cc(-c2ccc(N3c4cc(N(c5ccccc5)c5ccccc5)ccc4B4c5cnn(-c6ccccc6)c5N(c5ccccc5)c5cccc3c54)cc2)cc(C(C)(C)C)c1]

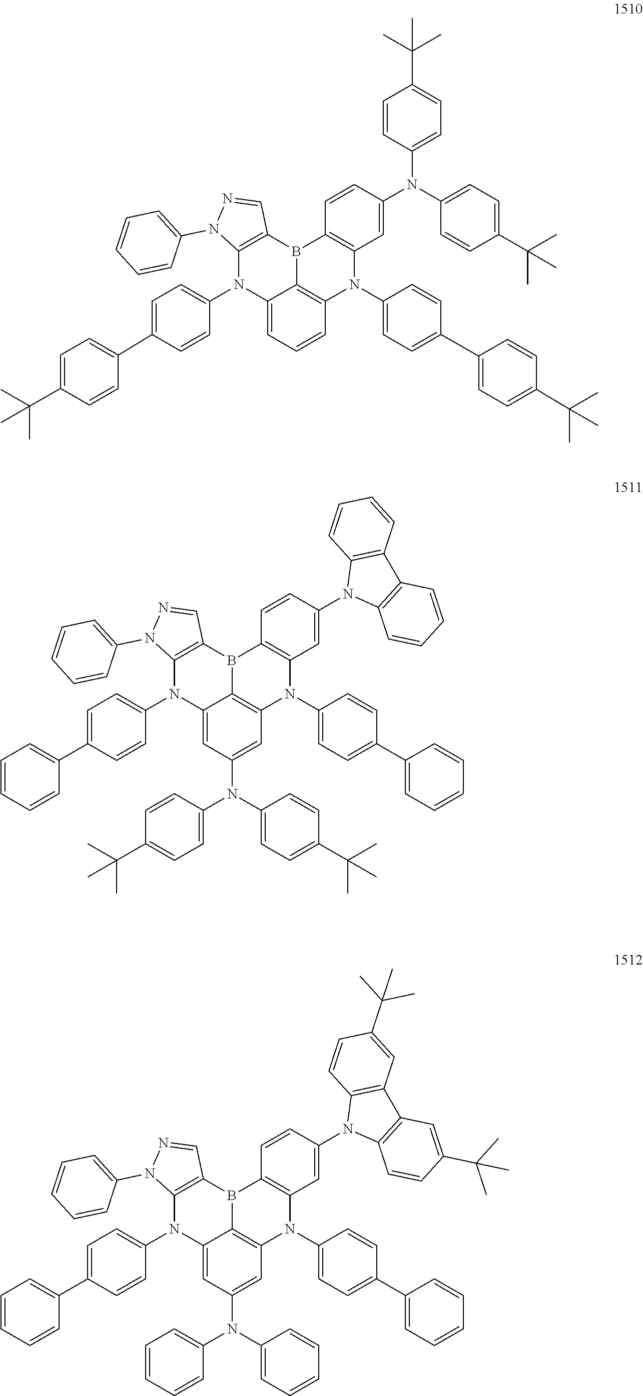 [SMILES: CC(C)(C)c1ccc(-c2ccc(N3c4cc(N(c5ccc(C(C)(C)C)cc5)c5ccc(C(C)(C)C)cc5)ccc4B4c5cnn(-c6ccccc6)c5N(c5ccc(-c6ccc(C(C)(C)C)cc6)cc5)c5cccc3c54)cc2)cc1 ; CC(C)(C)c1ccc(N(c2ccc(C(C)(C)C)cc2)c2cc3c4c(c2)N(c2ccc(-c5ccccc5)cc2)c2c(cnn2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1cnn(-c2ccccc2)c1N3c1ccc(-c2ccccc2)cc1]

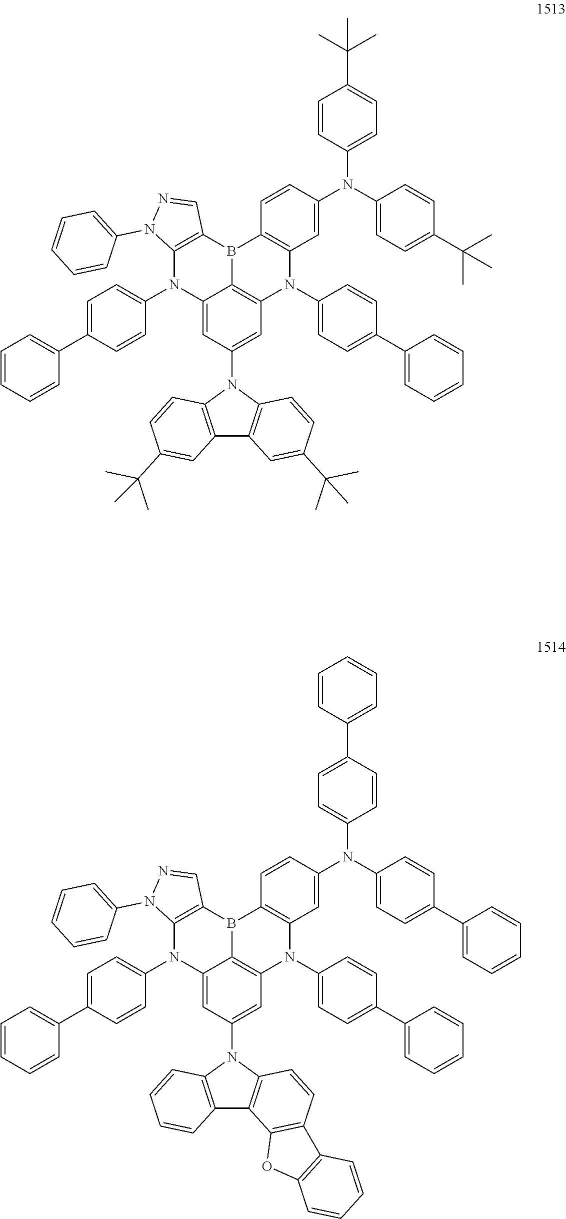 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2cnn(-c3ccccc3)c2N4c2ccc(-c3ccccc3)cc2)cc1 ; c1ccc(-c2ccc(N(c3ccc(-c4ccccc4)cc3)c3ccc4c(c3)N(c3ccc(-c5ccccc5)cc3)c3cc(-n5c6ccccc6c6c7oc8ccccc8c7ccc65)cc5c3B4c3cnn(-c4ccccc4)c3N5c3ccc(-c4ccccc4)cc3)cc2)cc1]

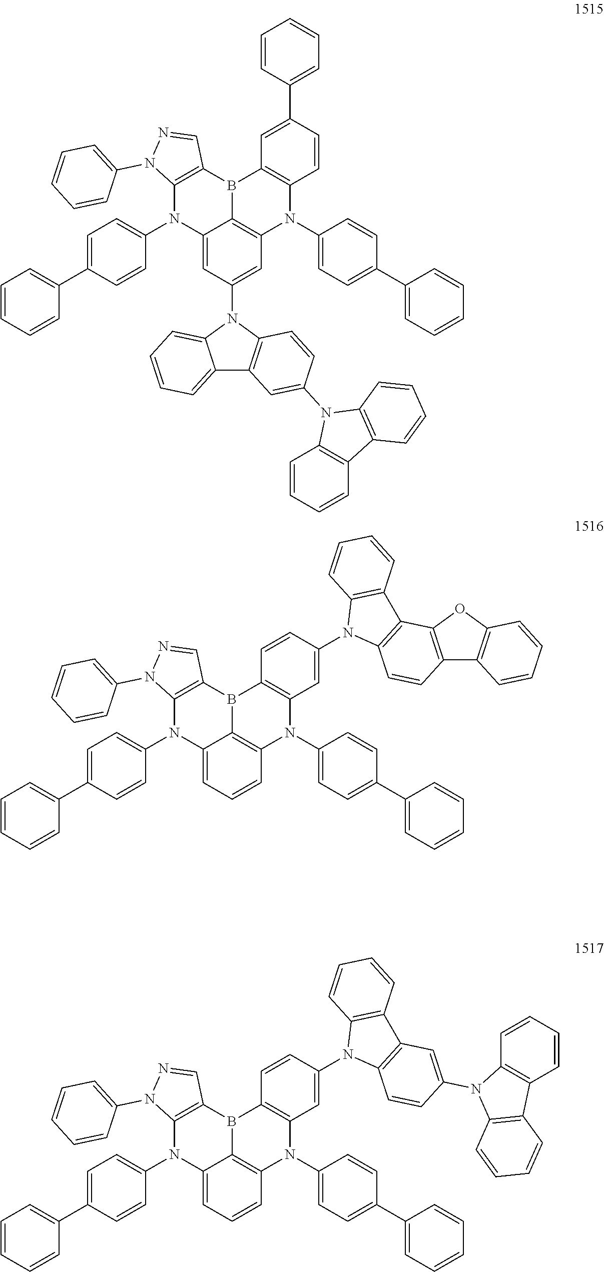

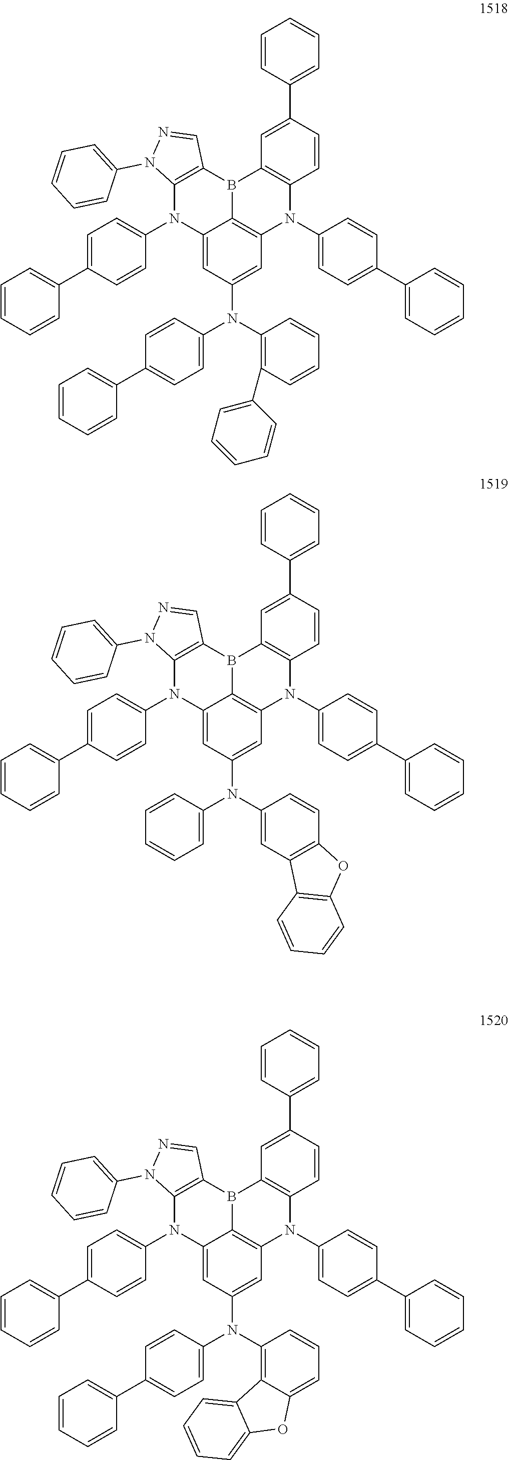

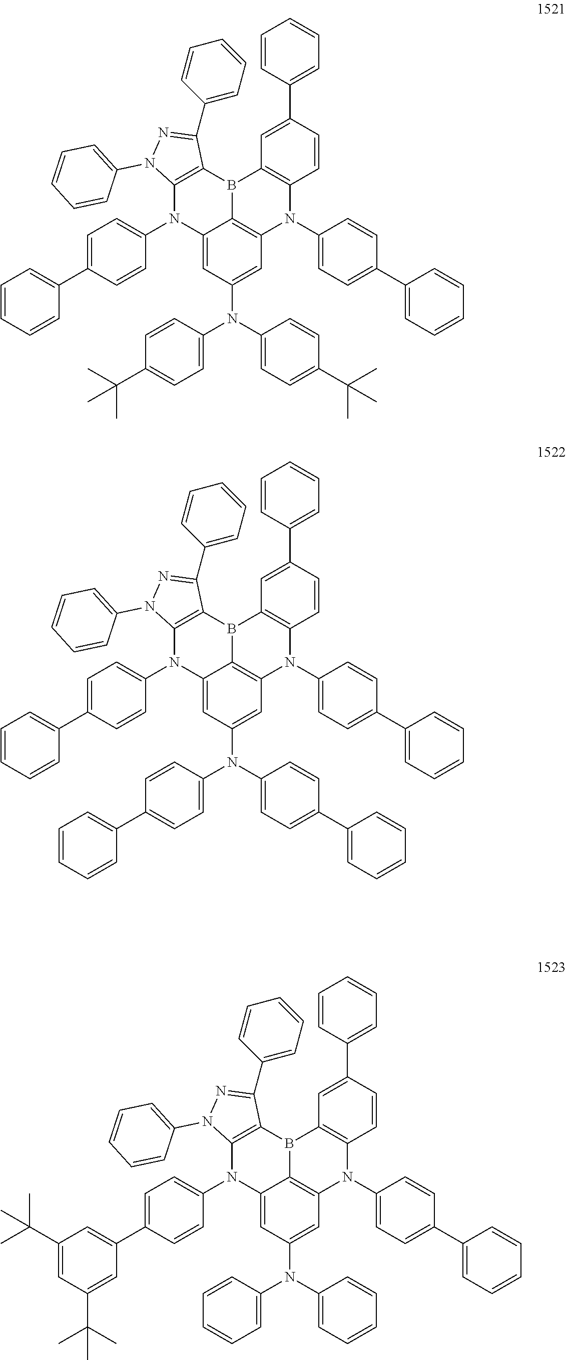 [SMILES: CC(C)(C)c1ccc(N(c2ccc(C(C)(C)C)cc2)c2cc3c4c(c2)N(c2ccc(-c5ccccc5)cc2)c2c(c(-c5ccccc5)nn2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(c(-c6ccccc6)nn3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c(-c5ccccc5)nn(-c5ccccc5)c43)cc2)cc(C(C)(C)C)c1]

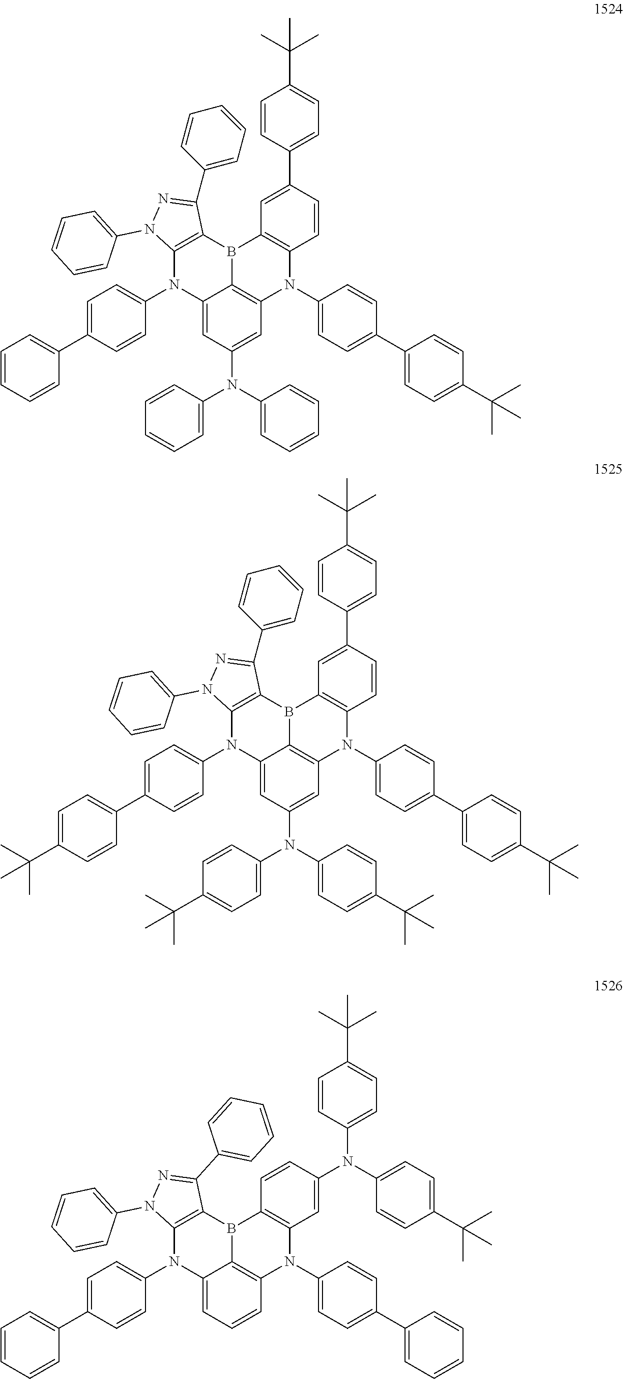

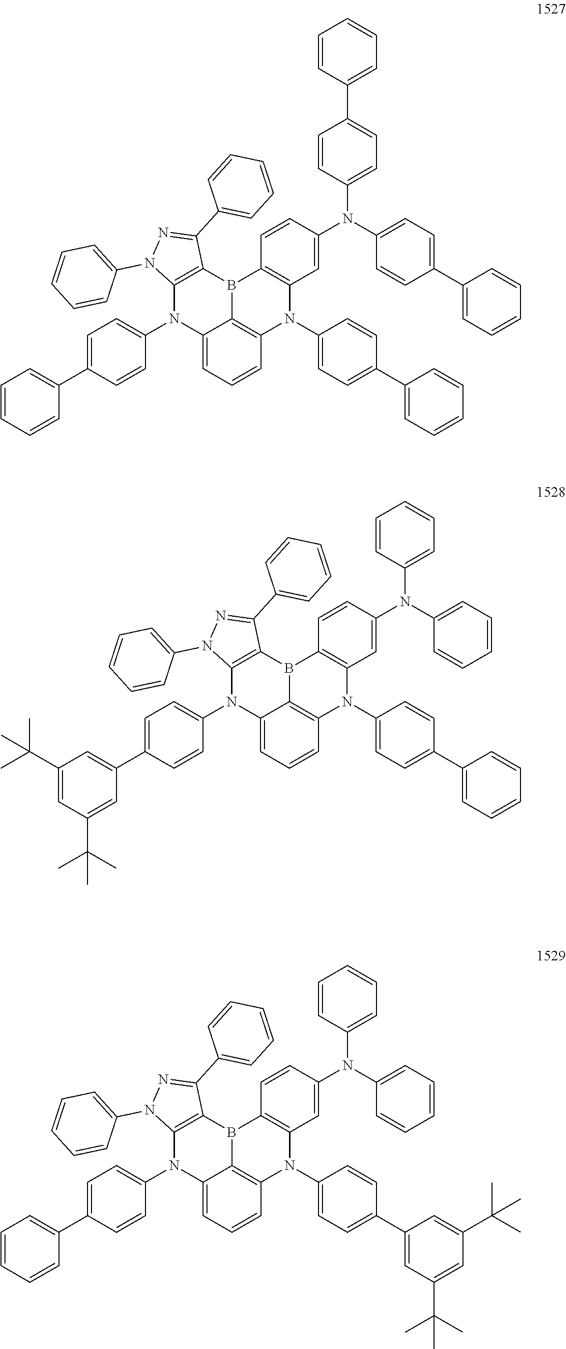 [SMILES: c1ccc(-c2ccc(N(c3ccc(-c4ccccc4)cc3)c3ccc4c(c3)N(c3ccc(-c5ccccc5)cc3)c3cccc5c3B4c3c(-c4ccccc4)nn(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4c(-c5ccccc5)nn(-c5ccccc5)c43)cc2)cc(C(C)(C)C)c1 ; CC(C)(C)c1cc(-c2ccc(N3c4cc(N(c5ccccc5)c5ccccc5)ccc4B4c5c3cccc5N(c3ccc(-c5ccccc5)cc3)c3c4c(-c4ccccc4)nn3-c3ccccc3)cc2)cc(C(C)(C)C)c1]

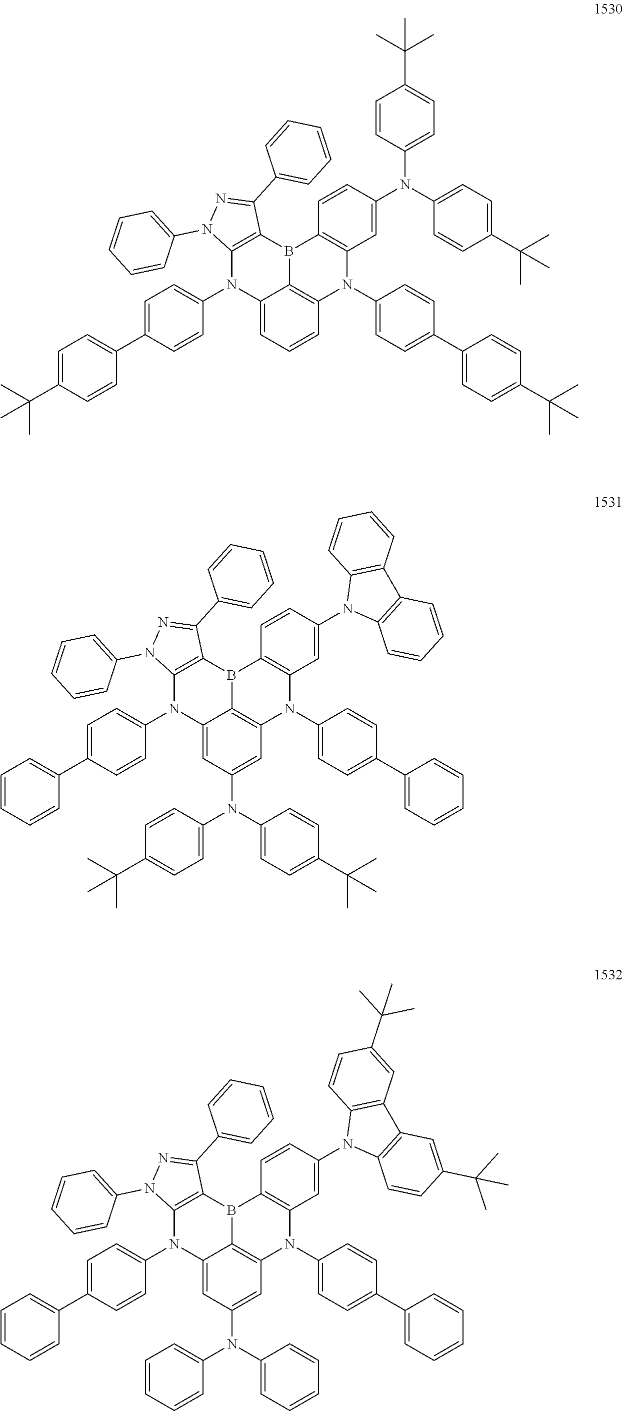 [SMILES: CC(C)(C)c1ccc(-c2ccc(N3c4cc(N(c5ccc(C(C)(C)C)cc5)c5ccc(C(C)(C)C)cc5)ccc4B4c5c3cccc5N(c3ccc(-c5ccc(C(C)(C)C)cc5)cc3)c3c4c(-c4ccccc4)nn3-c3ccccc3)cc2)cc1 ; CC(C)(C)c1ccc(N(c2ccc(C(C)(C)C)cc2)c2cc3c4c(c2)N(c2ccc(-c5ccccc5)cc2)c2c(c(-c5ccccc5)nn2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(-c2ccccc2)nn(-c2ccccc2)c1N3c1ccc(-c2ccccc2)cc1]

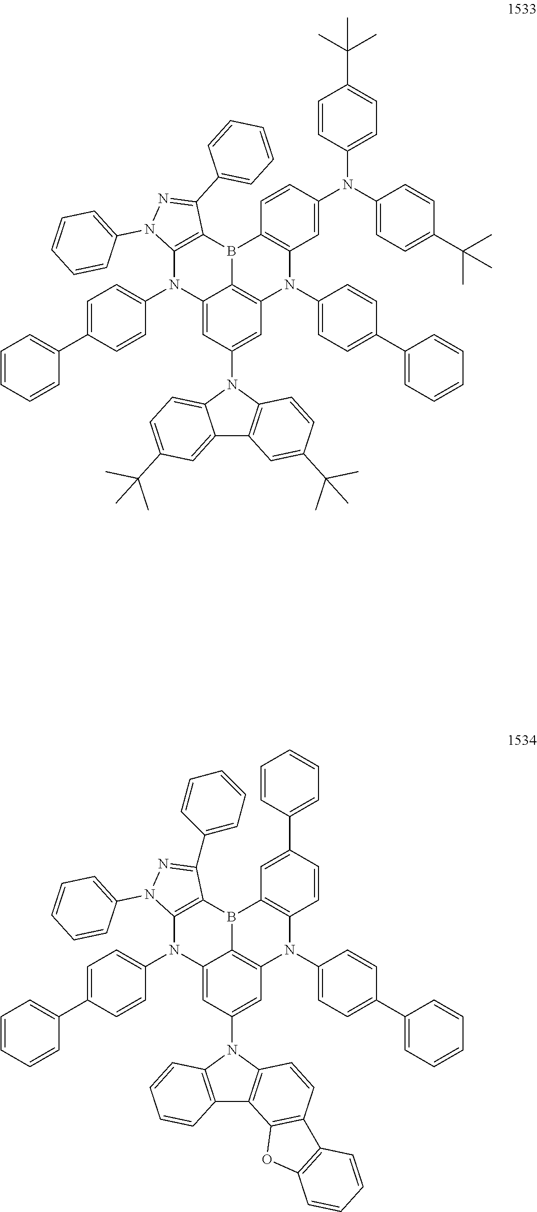 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2c(-c3ccccc3)nn(-c3ccccc3)c2N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3c4c(-c4ccccc4)nn3-c3ccccc3)cc2)cc1]

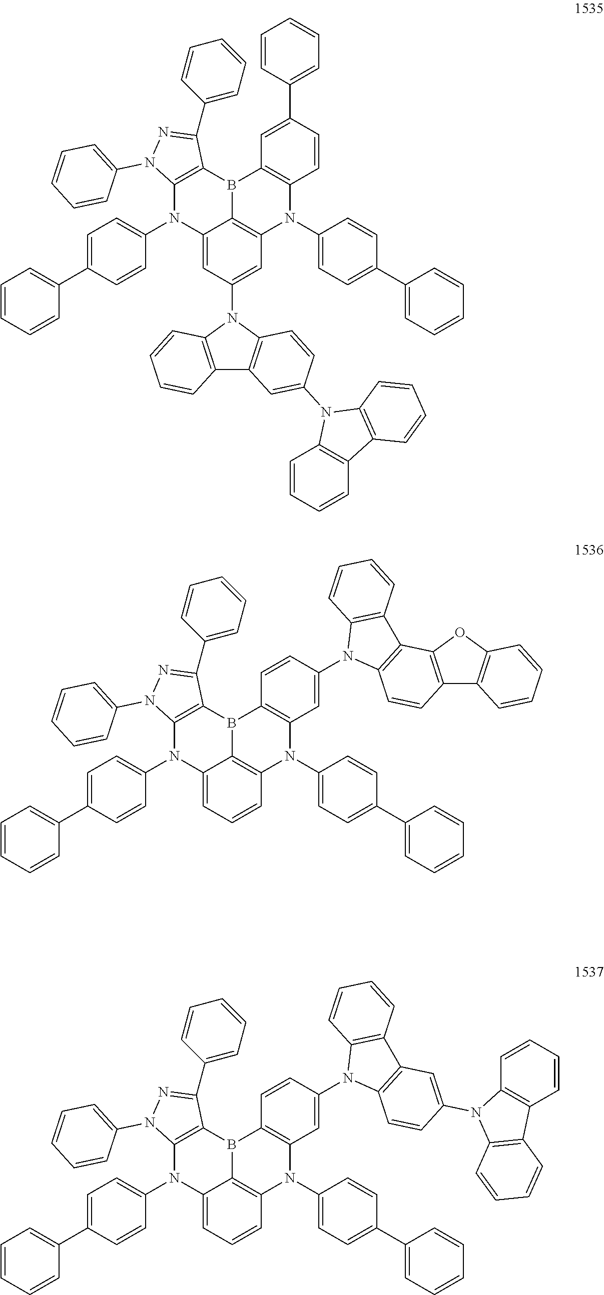 [SMILES: c1ccc(-c2ccc(N3c4ccc(-c5ccccc5)cc4B4c5c3cc(-n3c6ccccc6c6cc(-n7c8ccccc8c8ccccc87)ccc63)cc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6c7oc8ccccc8c7ccc65)ccc4B4c5c3cccc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3c4c(-c4ccccc4)nn3-c3ccccc3)cc2)cc1]

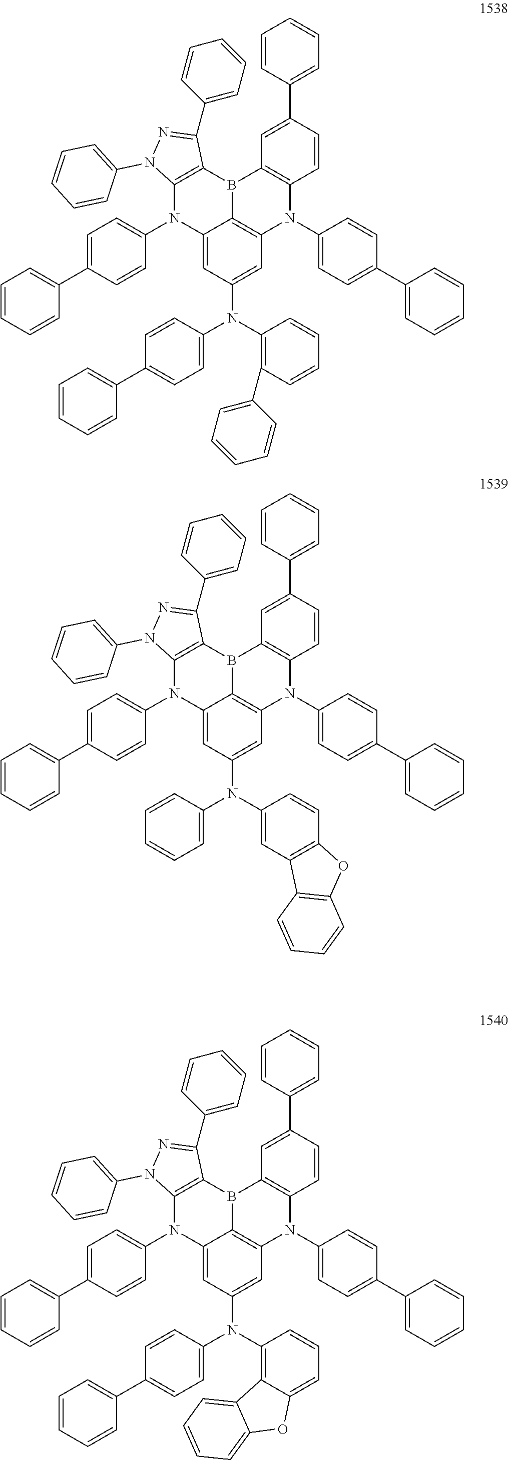

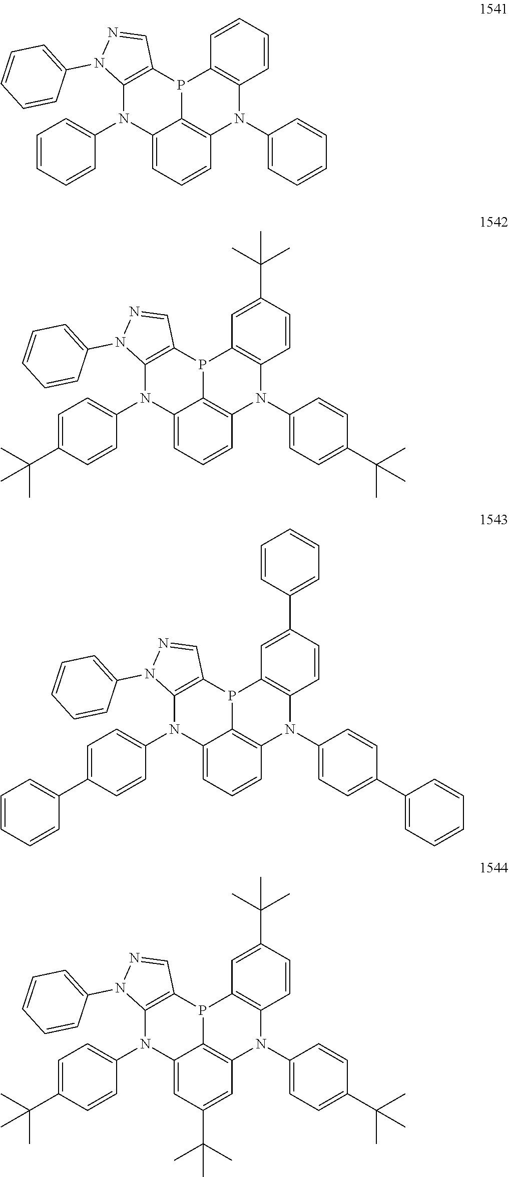

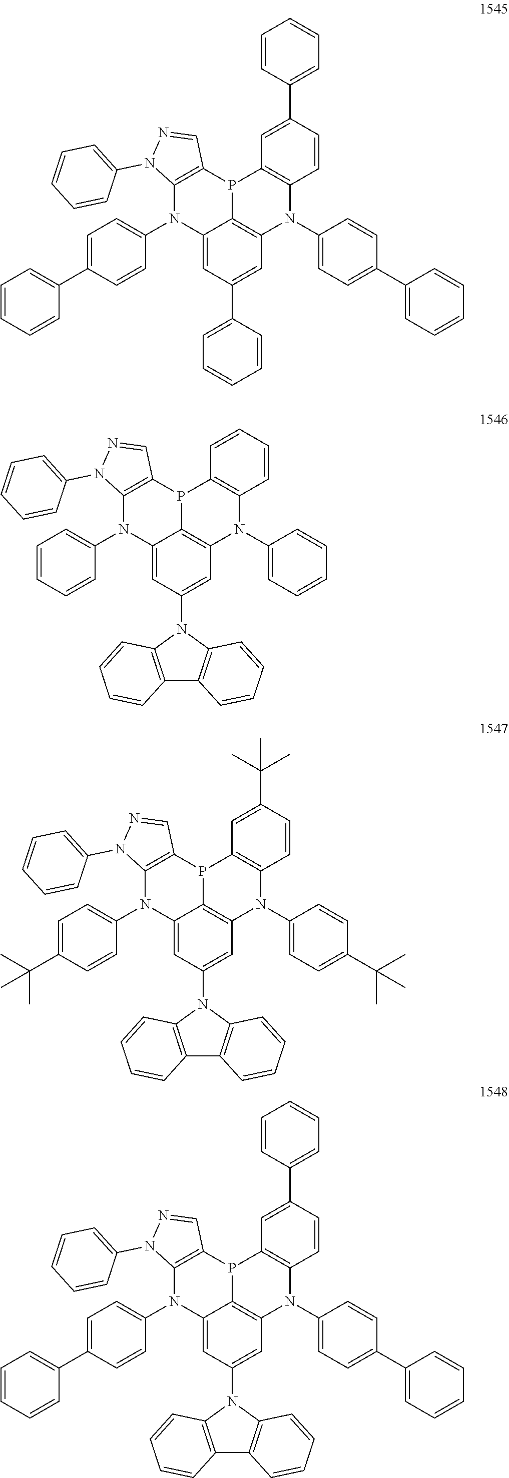

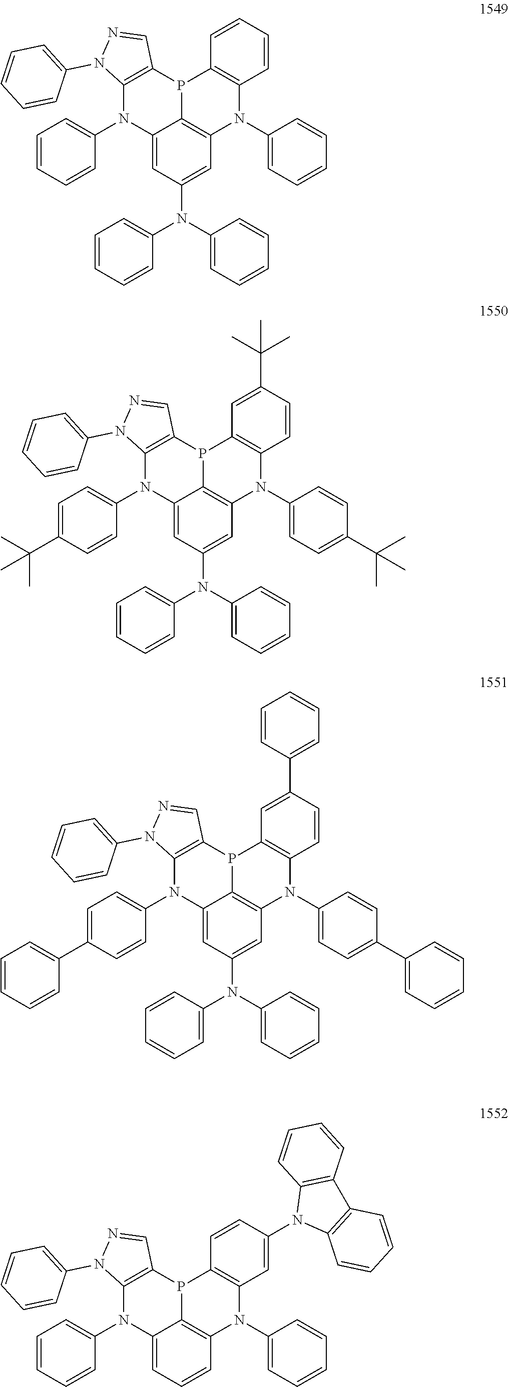

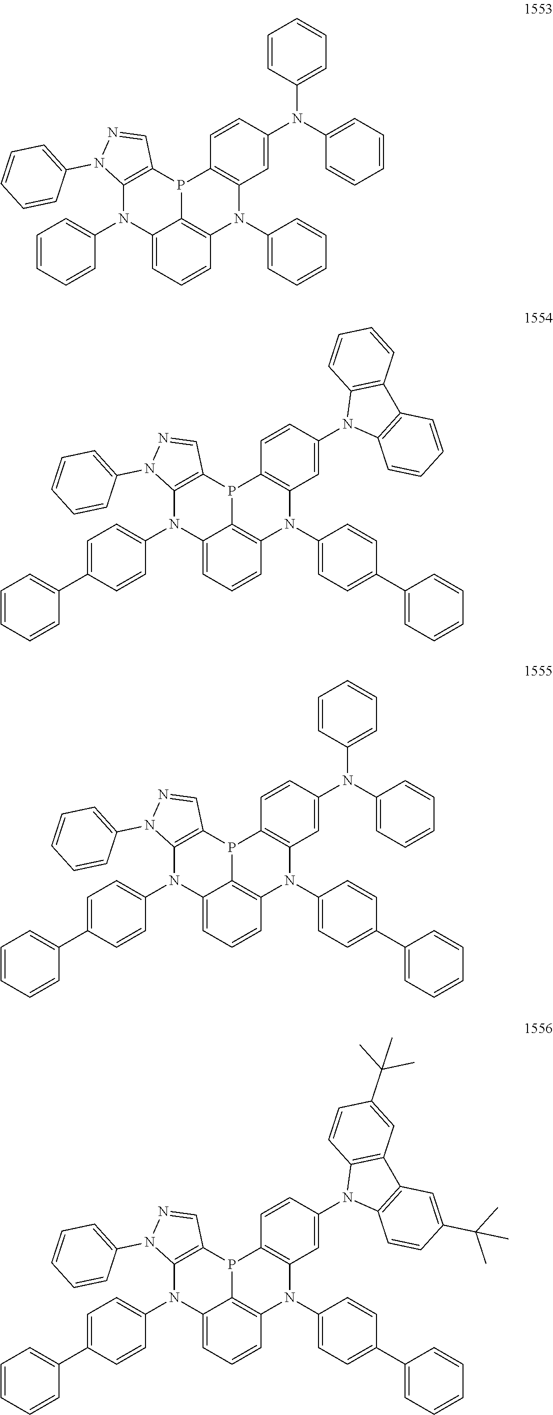 [SMILES: c1ccc(N(c2ccccc2)c2ccc3c(c2)N(c2ccccc2)c2cccc4c2P3c2cnn(-c3ccccc3)c2N4c2ccccc2)cc1 ; c1ccc(-c2ccc(N3c4cc(-n5c6ccccc6c6ccccc65)ccc4P4c5cnn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4P4c5cnn(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1cnn(-c2ccccc2)c1N3c1ccc(-c2ccccc2)cc1]

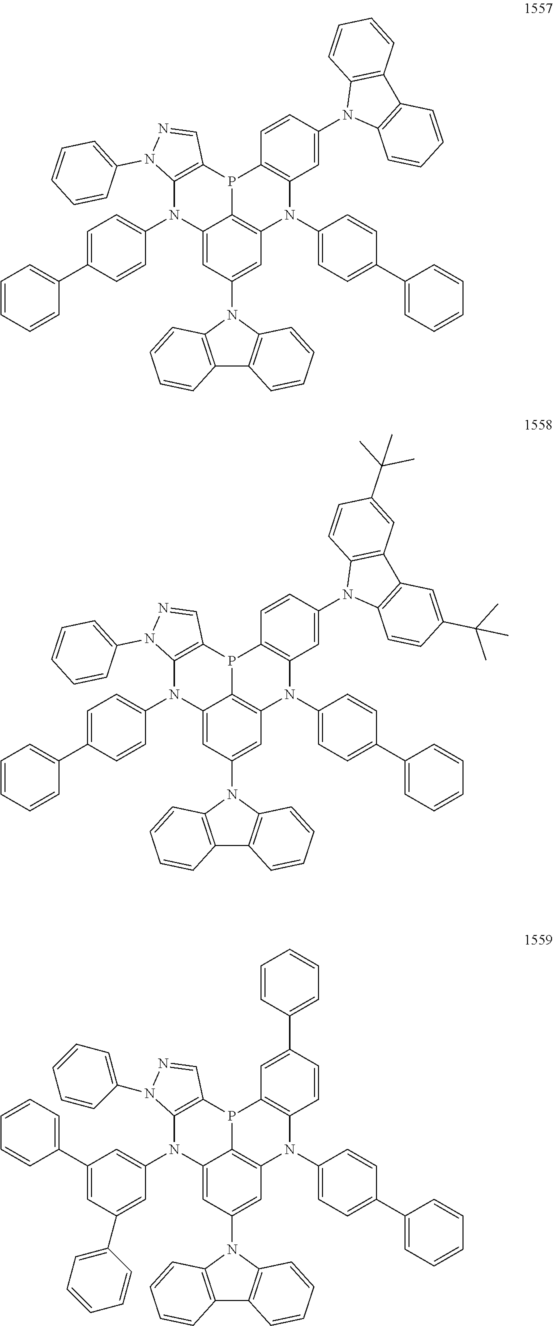 [SMILES: c1ccc(-c2ccc(N3c4cc(-n5c6ccccc6c6ccccc65)ccc4P4c5cnn(-c6ccccc6)c5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1cnn(-c2ccccc2)c1N3c1ccc(-c2ccccc2)cc1 ; c1ccc(-c2ccc(N3c4ccc(-c5ccccc5)cc4P4c5cnn(-c6ccccc6)c5N(c5cc(-c6ccccc6)cc(-c6ccccc6)c5)c5cc(-n6c7ccccc7c7ccccc76)cc3c54)cc2)cc1]

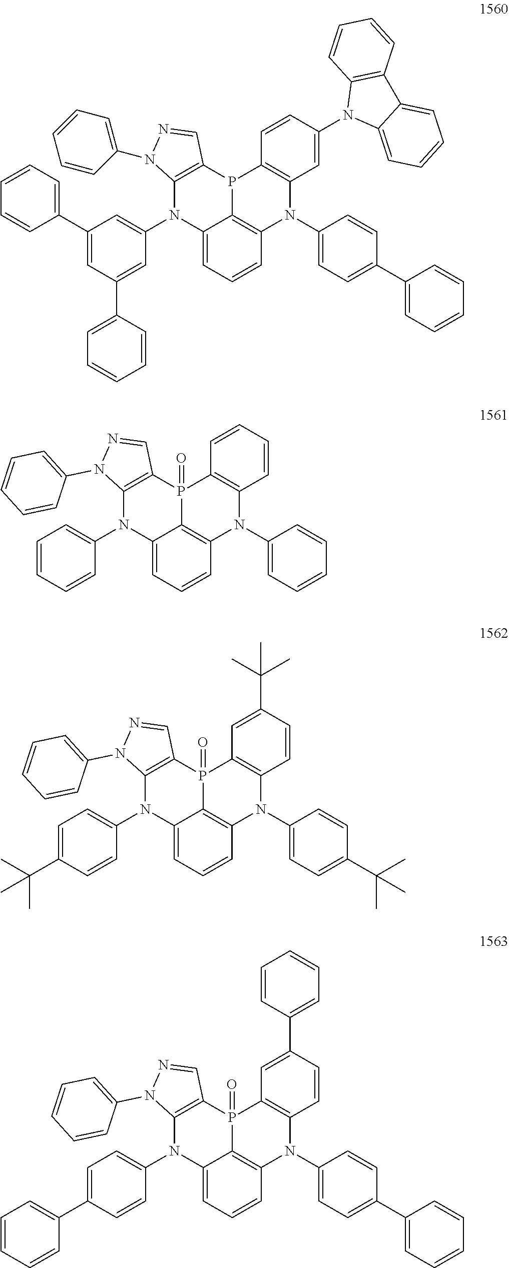 [SMILES: c1ccc(-c2ccc(N3c4cc(-n5c6ccccc6c6ccccc65)ccc4P4c5cnn(-c6ccccc6)c5N(c5cc(-c6ccccc6)cc(-c6ccccc6)c5)c5cccc3c54)cc2)cc1 ; O=P12c3ccccc3N(c3ccccc3)c3cccc(c31)N(c1ccccc1)c1c2cnn1-c1ccccc1 ; CC(C)(C)c1ccc(N2c3ccc(C(C)(C)C)cc3P3(=O)c4cnn(-c5ccccc5)c4N(c4ccc(C(C)(C)C)cc4)c4cccc2c43)cc1 ; O=P12c3cc(-c4ccccc4)ccc3N(c3ccc(-c4ccccc4)cc3)c3cccc(c31)N(c1ccc(-c3ccccc3)cc1)c1c2cnn1-c1ccccc1]

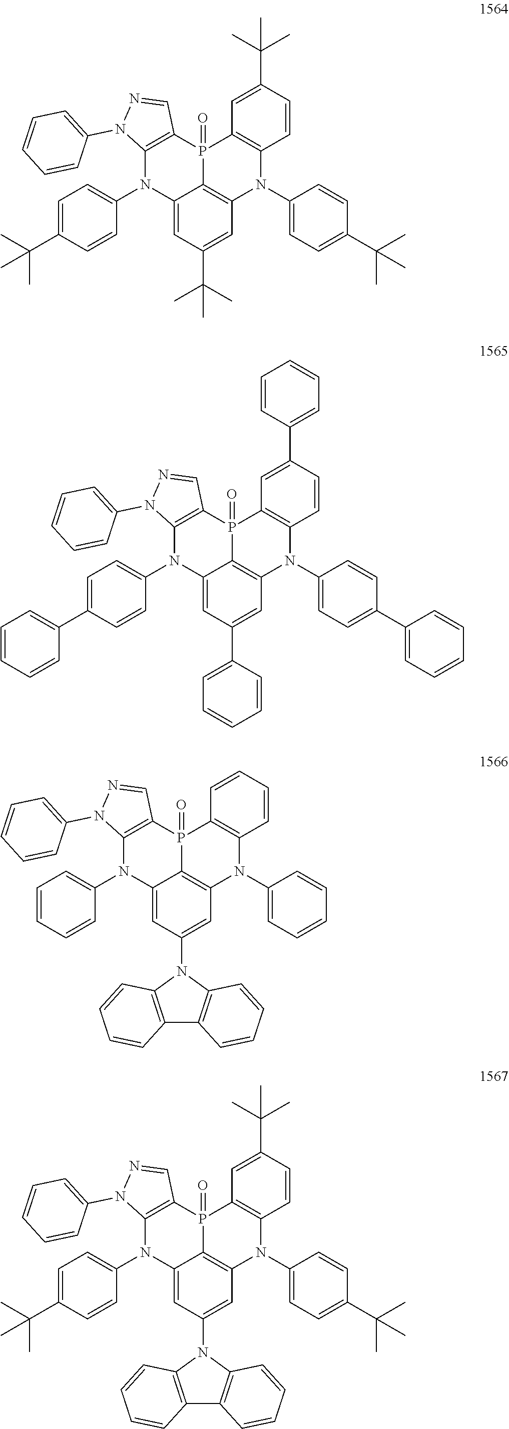

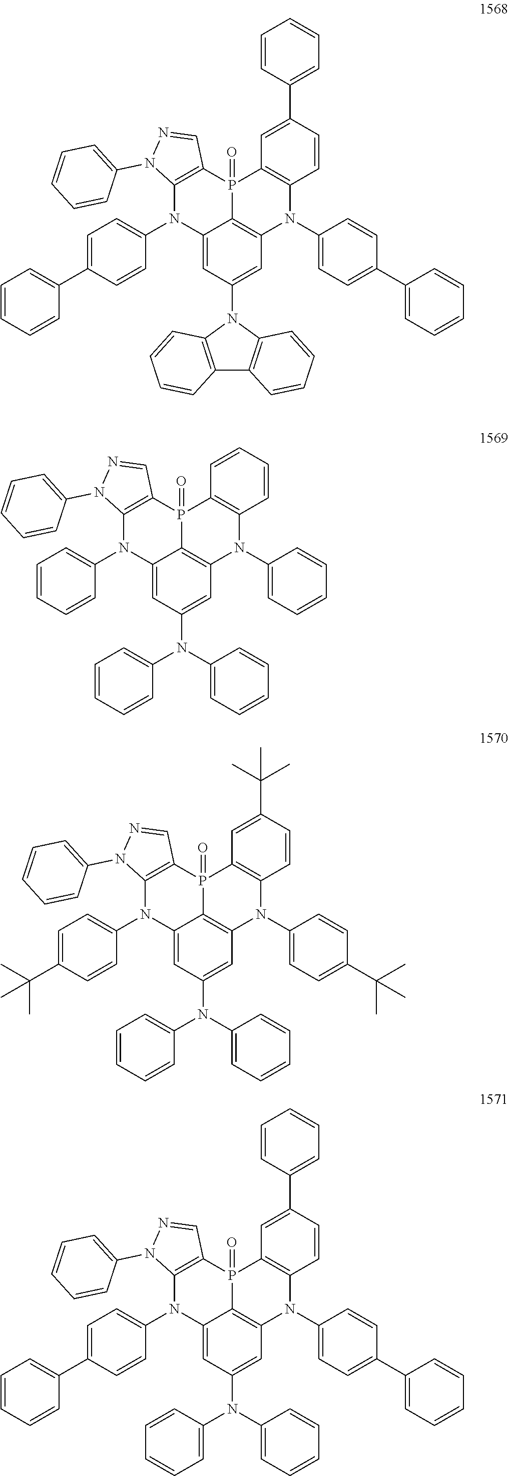

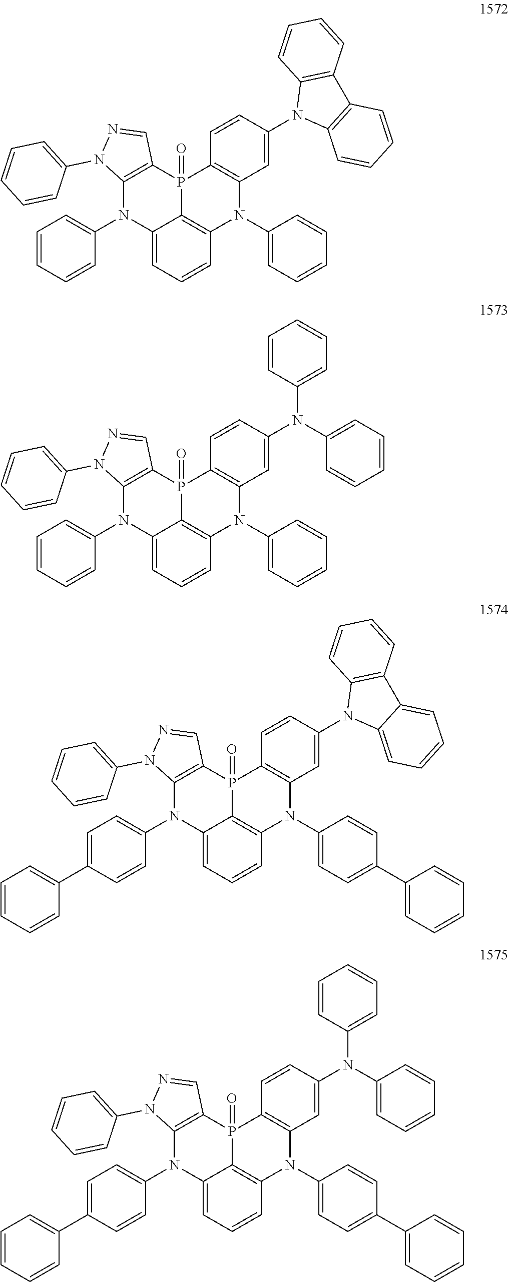

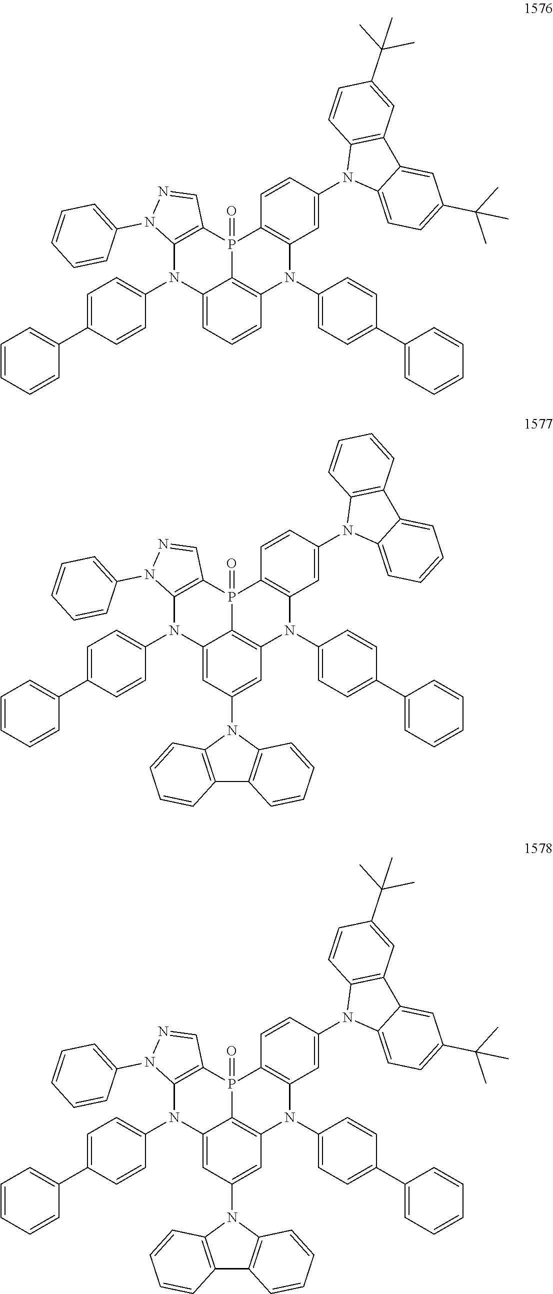 [SMILES: CC(C)(C)c1ccc2c(c1)c1cc(C(C)(C)C)ccc1n2-c1ccc2c(c1)N(c1ccc(-c3ccccc3)cc1)c1cccc3c1P2(=O)c1cnn(-c2ccccc2)c1N3c1ccc(-c2ccccc2)cc1 ; O=P12c3ccc(-n4c5ccccc5c5ccccc54)cc3N(c3ccc(-c4ccccc4)cc3)c3cc(-n4c5ccccc5c5ccccc54)cc(c31)N(c1ccc(-c3ccccc3)cc1)c1c2cnn1-c1ccccc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2(=O)c1cnn(-c2ccccc2)c1N3c1ccc(-c2ccccc2)cc1]

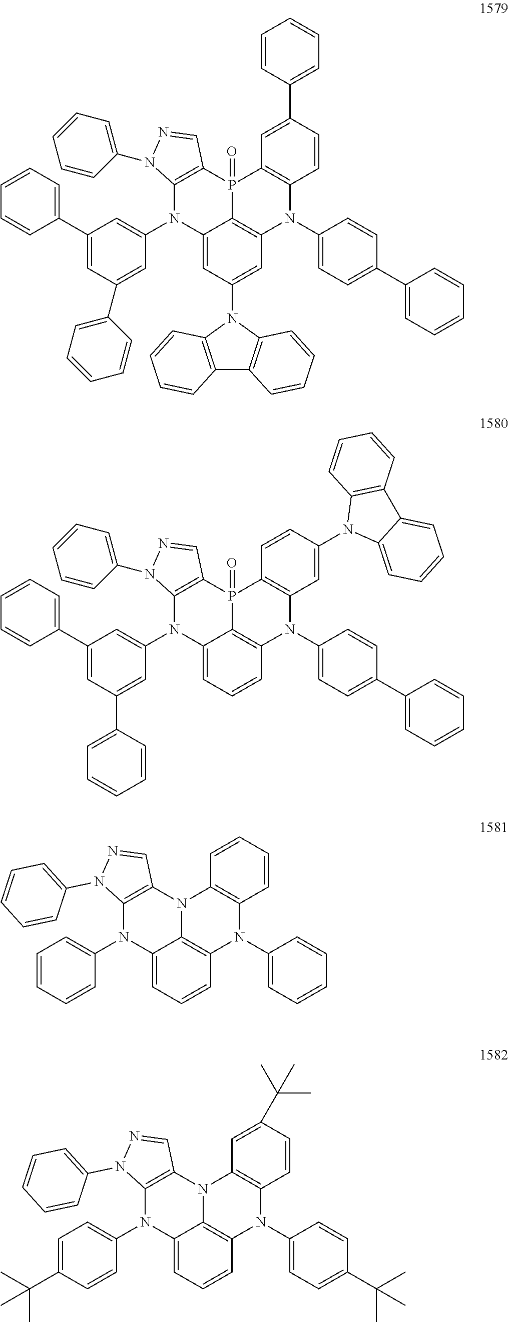 [SMILES: O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1c2cnn1-c1ccccc1 ; O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1c2cnn1-c1ccccc1 ; c1ccc(N2c3ccccc3N3c4cnn(-c5ccccc5)c4N(c4ccccc4)c4cccc2c43)cc1 ; CC(C)(C)c1ccc(N2c3ccc(C(C)(C)C)cc3N3c4cnn(-c5ccccc5)c4N(c4ccc(C(C)(C)C)cc4)c4cccc2c43)cc1]

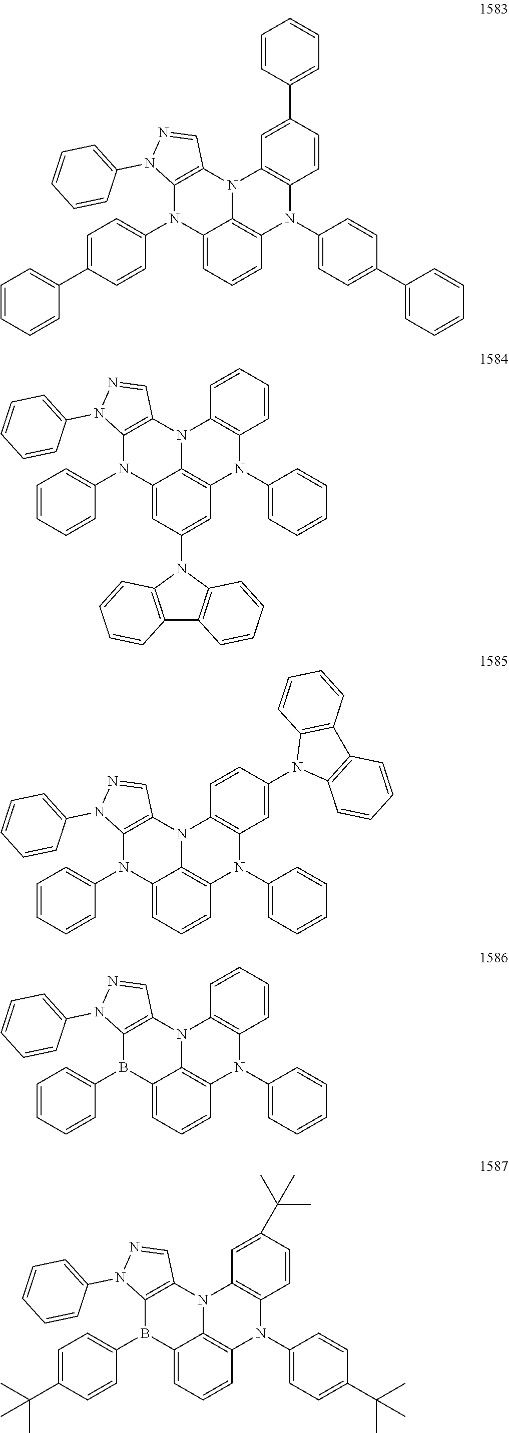

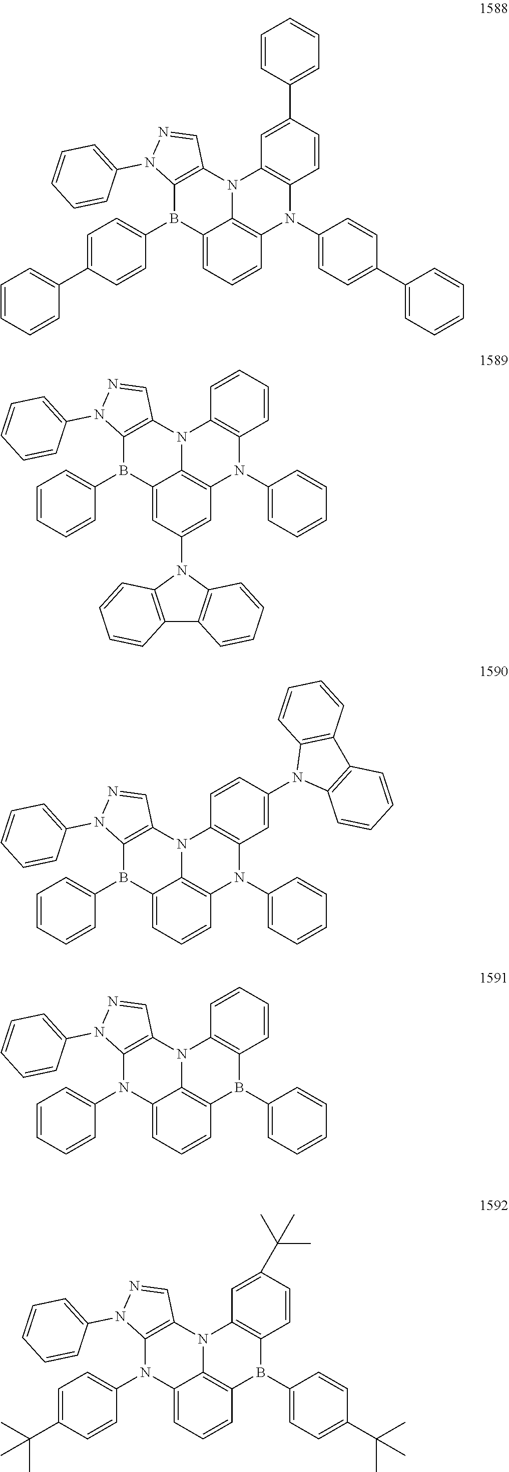 [SMILES: c1ccc(-c2ccc(B3c4cccc5c4N(c4cc(-c6ccccc6)ccc4N5c4ccc(-c5ccccc5)cc4)c4cnn(-c5ccccc5)c43)cc2)cc1 ; c1ccc(B2c3cc(-n4c5ccccc5c5ccccc54)cc4c3N(c3ccccc3N4c3ccccc3)c3cnn(-c4ccccc4)c32)cc1 ; c1ccc(B2c3cccc4c3N(c3ccc(-n5c6ccccc6c6ccccc65)cc3N4c3ccccc3)c3cnn(-c4ccccc4)c32)cc1 ; c1ccc(B2c3ccccc3N3c4cnn(-c5ccccc5)c4N(c4ccccc4)c4cccc2c43)cc1 ; CC(C)(C)c1ccc(B2c3ccc(C(C)(C)C)cc3N3c4cnn(-c5ccccc5)c4N(c4ccc(C(C)(C)C)cc4)c4cccc2c43)cc1]

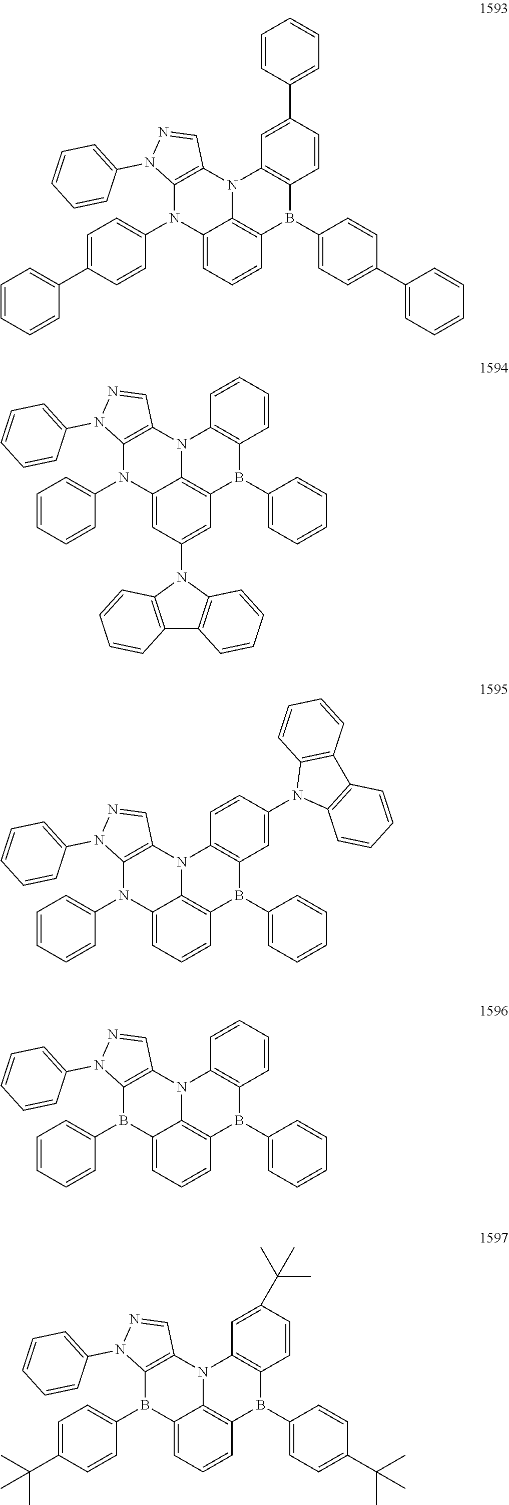 [SMILES: c1ccc(-c2ccc(B3c4ccc(-c5ccccc5)cc4N4c5cnn(-c6ccccc6)c5N(c5ccc(-c6ccccc6)cc5)c5cccc3c54)cc2)cc1 ; c1ccc(B2c3ccccc3N3c4cnn(-c5ccccc5)c4N(c4ccccc4)c4cc(-n5c6ccccc6c6ccccc65)cc2c43)cc1 ; c1ccc(B2c3cc(-n4c5ccccc5c5ccccc54)ccc3N3c4cnn(-c5ccccc5)c4N(c4ccccc4)c4cccc2c43)cc1 ; c1ccc(B2c3ccccc3N3c4cnn(-c5ccccc5)c4B(c4ccccc4)c4cccc2c43)cc1 ; CC(C)(C)c1ccc(B2c3ccc(C(C)(C)C)cc3N3c4cnn(-c5ccccc5)c4B(c4ccc(C(C)(C)C)cc4)c4cccc2c43)cc1]

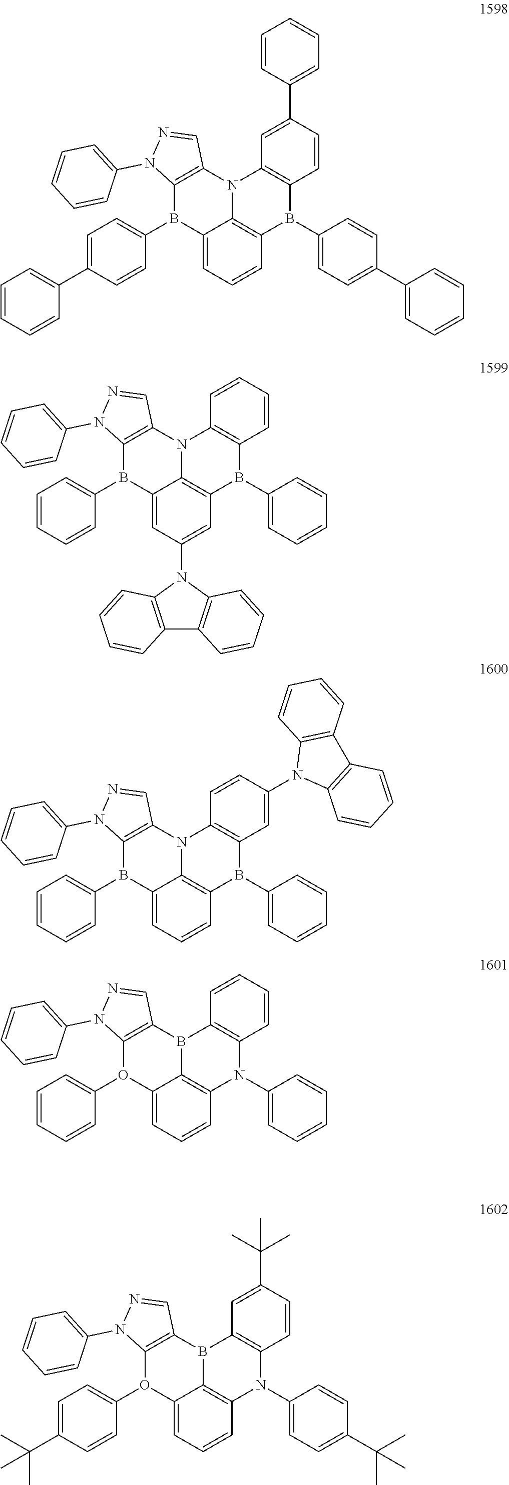

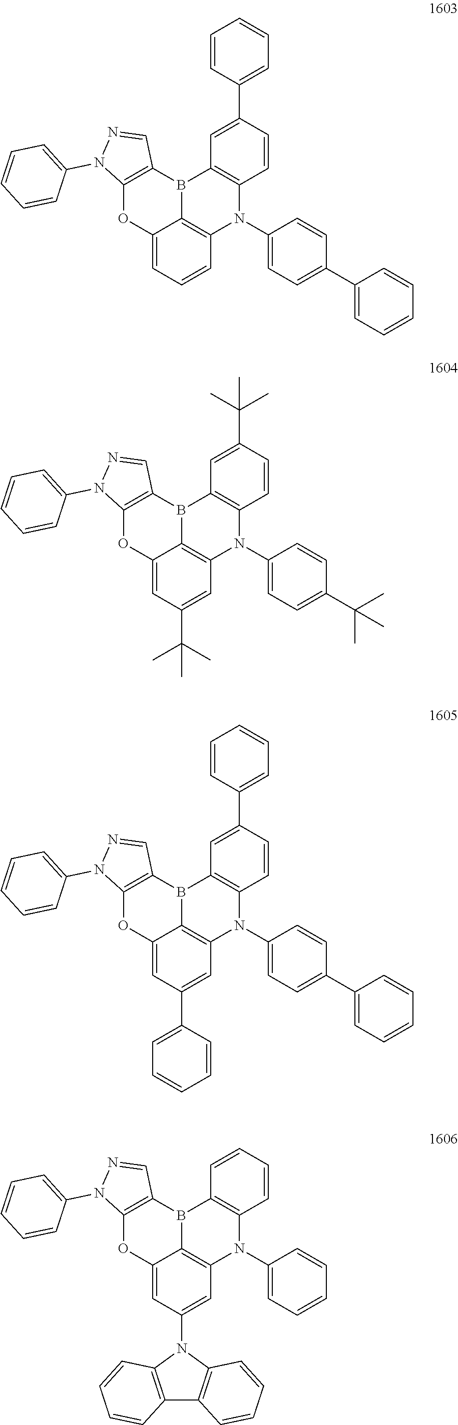 [SMILES: c1ccc(-c2ccc(N3c4ccc(-c5ccccc5)cc4B4c5cnn(-c6ccccc6)c5Oc5cccc3c54)cc2)cc1 ; CC(C)(C)c1ccc(N2c3ccc(C(C)(C)C)cc3B3c4cnn(-c5ccccc5)c4Oc4cc(C(C)(C)C)cc2c43)cc1 ; c1ccc(-c2ccc(N3c4ccc(-c5ccccc5)cc4B4c5cnn(-c6ccccc6)c5Oc5cc(-c6ccccc6)cc3c54)cc2)cc1 ; c1ccc(N2c3ccccc3B3c4cnn(-c5ccccc5)c4Oc4cc(-n5c6ccccc6c6ccccc65)cc2c43)cc1]

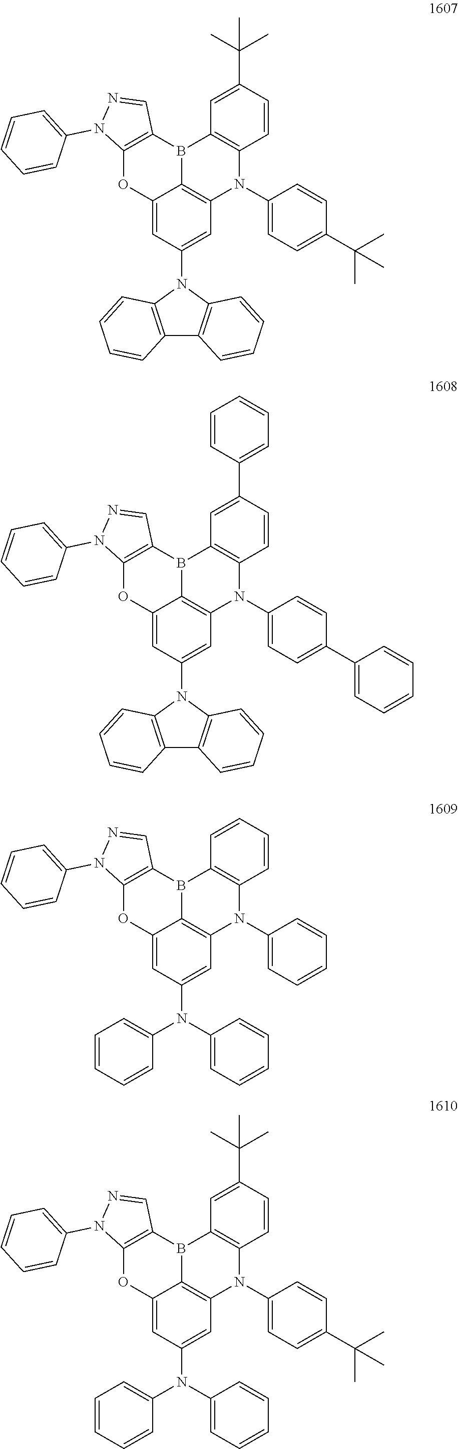

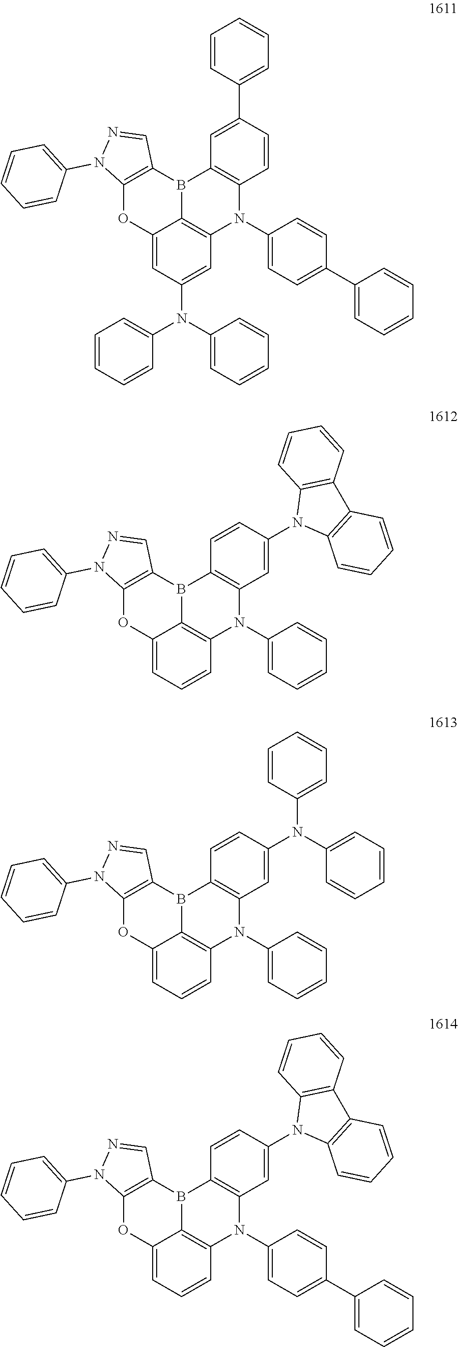

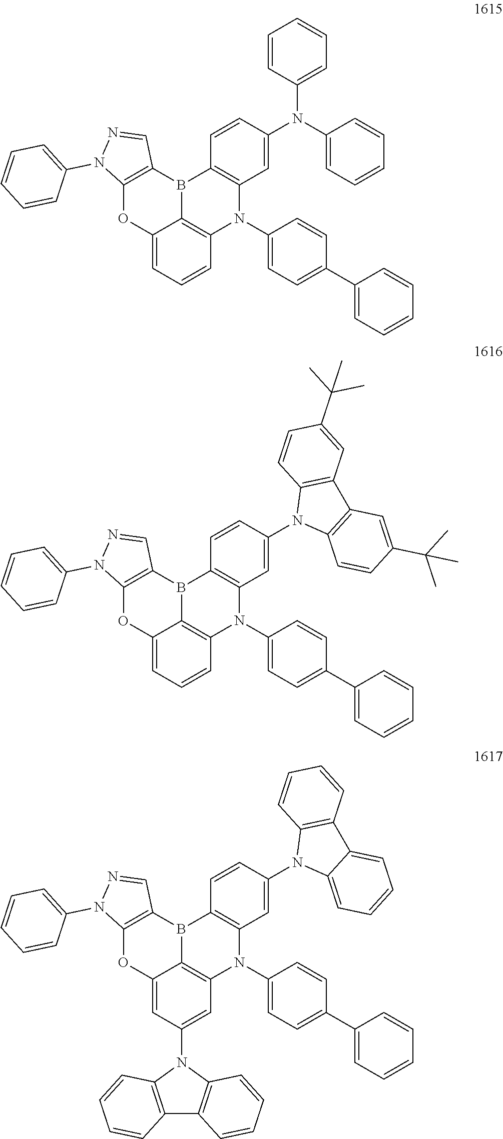

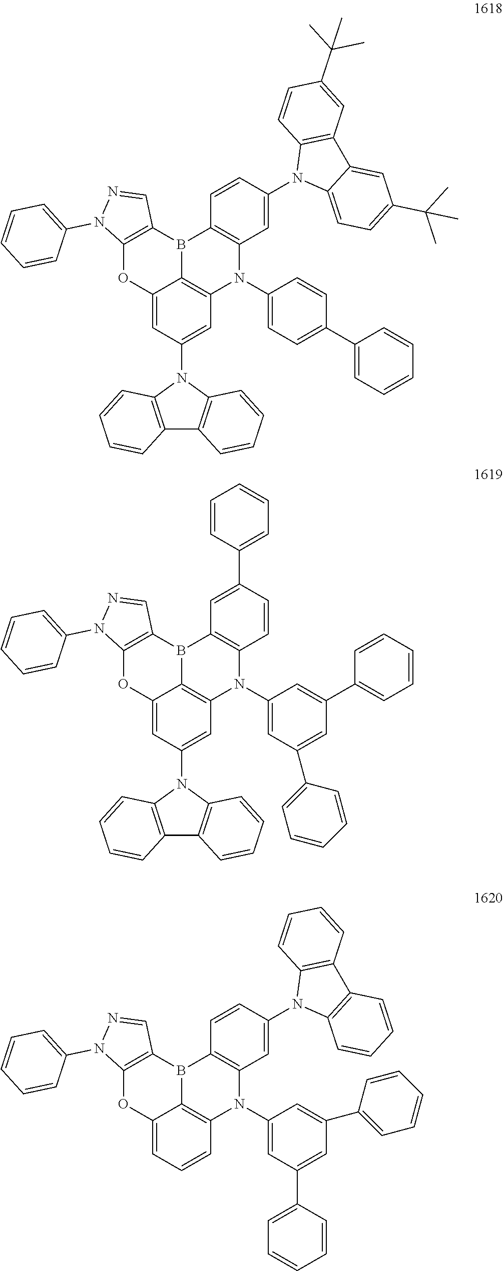

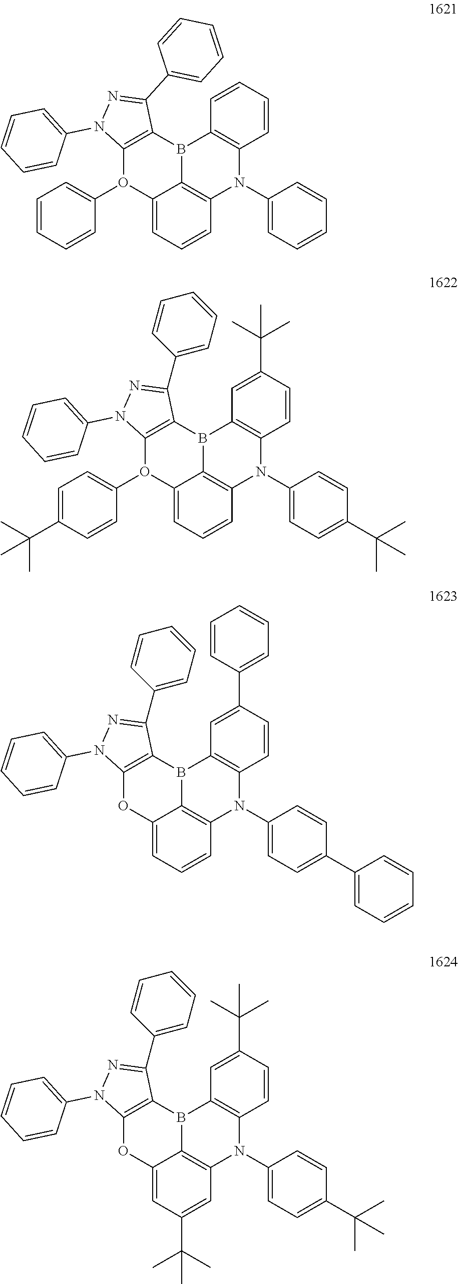

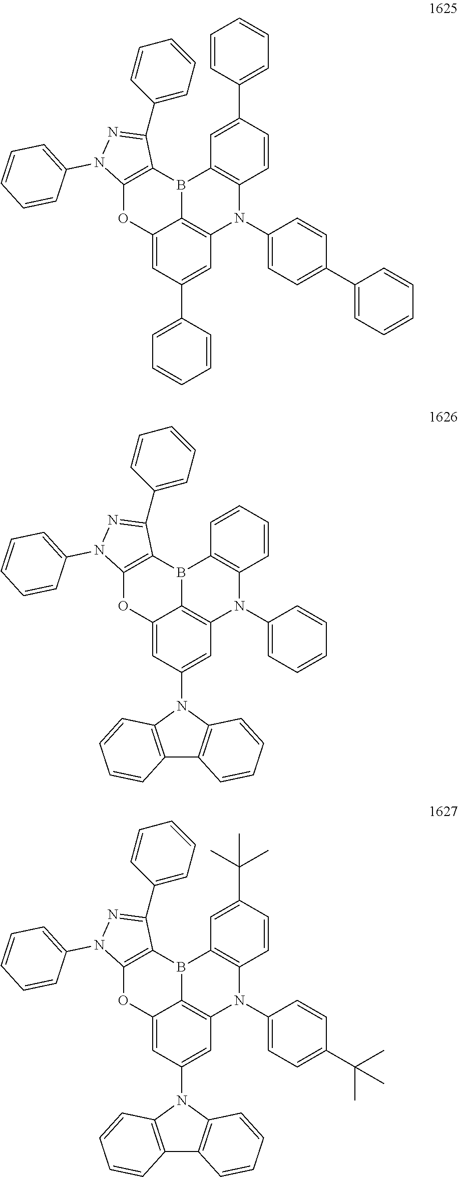

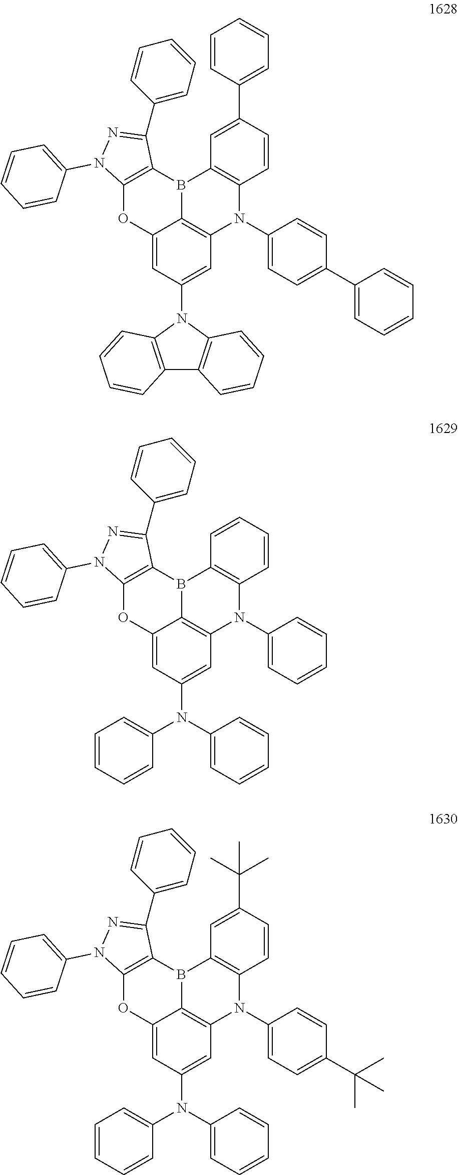 [SMILES: c1ccc(-c2ccc(N3c4ccc(-c5ccccc5)cc4B4c5c(cc(-n6c7ccccc7c7ccccc76)cc53)Oc3c4c(-c4ccccc4)nn3-c3ccccc3)cc2)cc1 ; c1ccc(-c2nn(-c3ccccc3)c3c2B2c4ccccc4N(c4ccccc4)c4cc(N(c5ccccc5)c5ccccc5)cc(c42)O3)cc1 ; CC(C)(C)c1ccc(N2c3ccc(C(C)(C)C)cc3B3c4c(cc(N(c5ccccc5)c5ccccc5)cc42)Oc2c3c(-c3ccccc3)nn2-c2ccccc2)cc1]

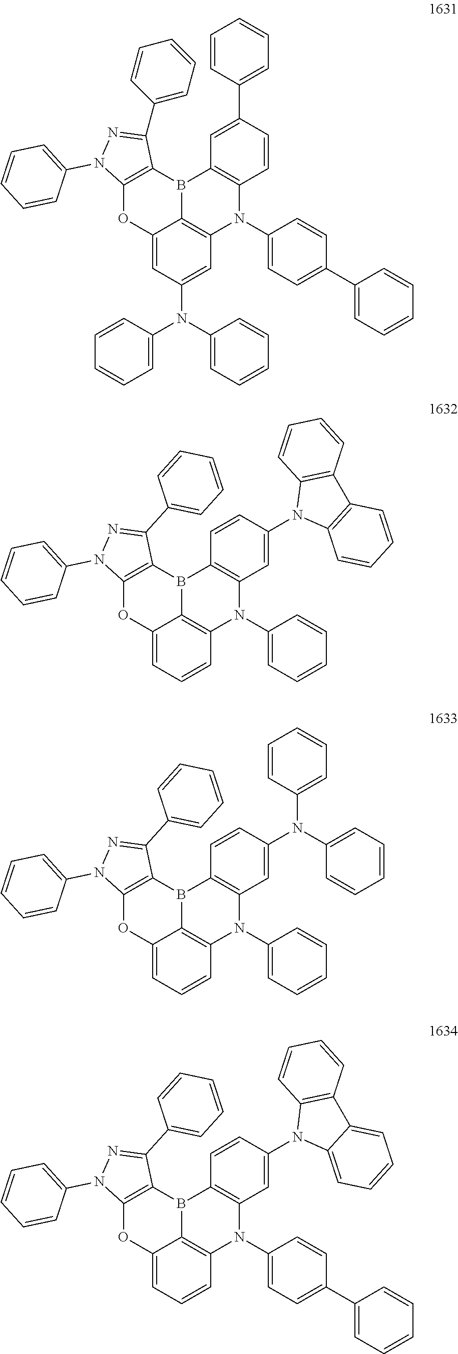

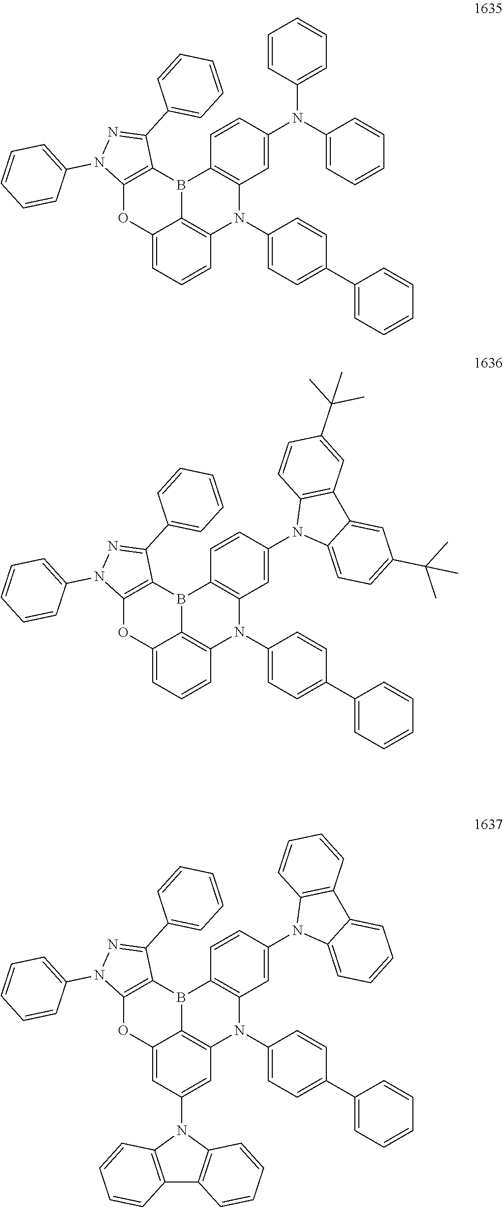 [SMILES: c1ccc(-c2ccc(N3c4cc(N(c5ccccc5)c5ccccc5)ccc4B4c5c(cccc53)Oc3c4c(-c4ccccc4)nn3-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(-c2ccccc2)nn(-c2ccccc2)c1O3 ; c1ccc(-c2ccc(N3c4cc(-n5c6ccccc6c6ccccc65)ccc4B4c5c(cc(-n6c7ccccc7c7ccccc76)cc53)Oc3c4c(-c4ccccc4)nn3-c3ccccc3)cc2)cc1]

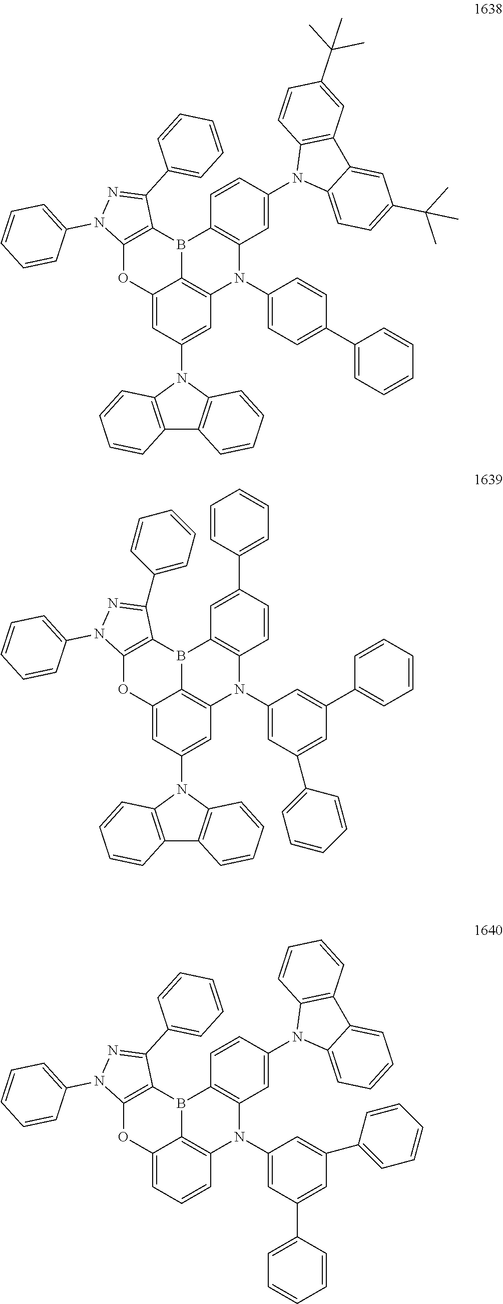

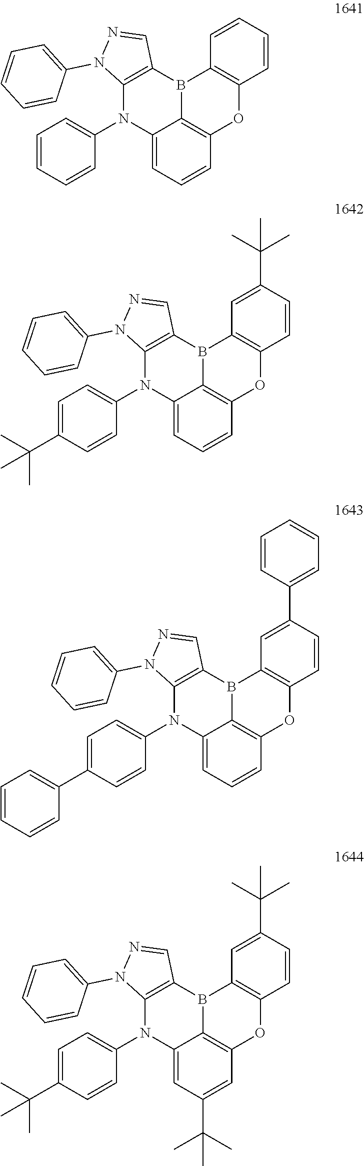

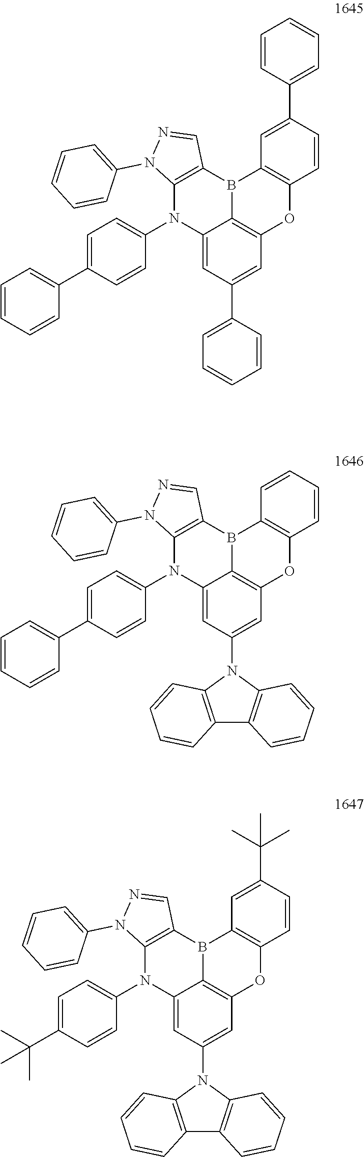 [SMILES: c1ccc(-c2ccc(N3c4cc(-c5ccccc5)cc5c4B(c4cc(-c6ccccc6)ccc4O5)c4cnn(-c5ccccc5)c43)cc2)cc1 ; c1ccc(-c2ccc(N3c4cc(-n5c6ccccc6c6ccccc65)cc5c4B(c4ccccc4O5)c4cnn(-c5ccccc5)c43)cc2)cc1 ; CC(C)(C)c1ccc(N2c3cc(-n4c5ccccc5c5ccccc54)cc4c3B(c3cc(C(C)(C)C)ccc3O4)c3cnn(-c4ccccc4)c32)cc1]

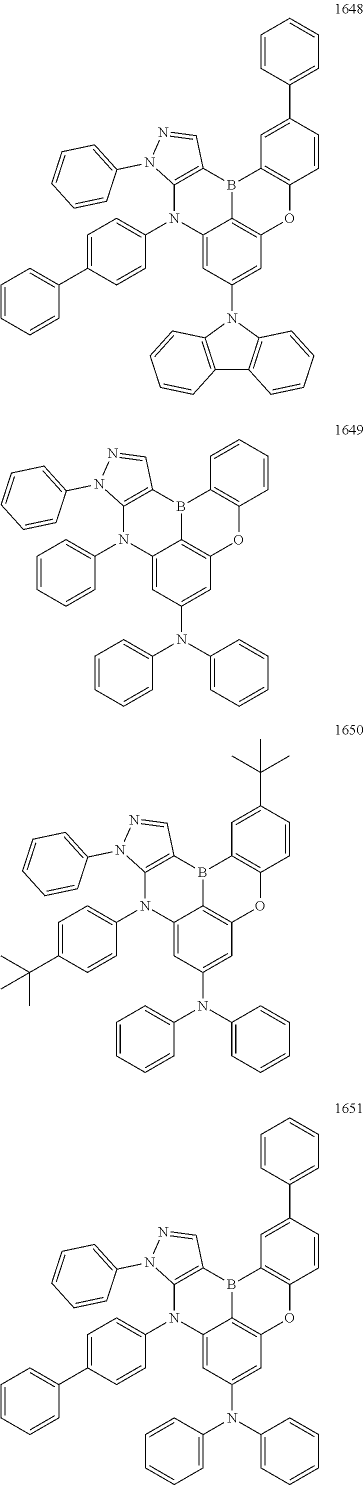

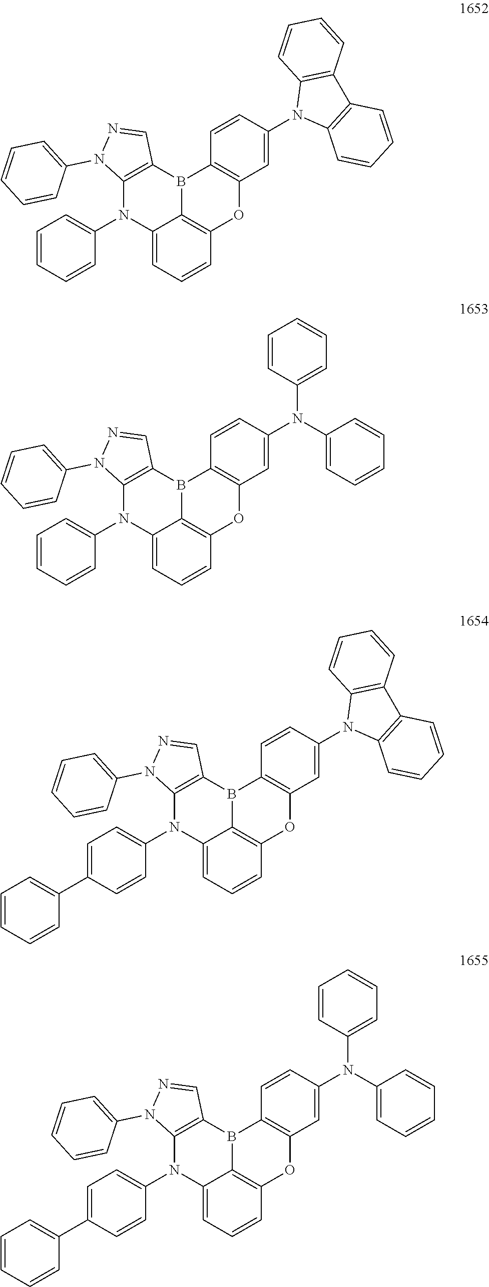 [SMILES: c1ccc(N2c3cccc4c3B(c3ccc(-n5c6ccccc6c6ccccc65)cc3O4)c3cnn(-c4ccccc4)c32)cc1 ; c1ccc(N(c2ccccc2)c2ccc3c(c2)Oc2cccc4c2B3c2cnn(-c3ccccc3)c2N4c2ccccc2)cc1 ; c1ccc(-c2ccc(N3c4cccc5c4B(c4ccc(-n6c7ccccc7c7ccccc76)cc4O5)c4cnn(-c5ccccc5)c43)cc2)cc1 ; c1ccc(-c2ccc(N3c4cccc5c4B(c4ccc(N(c6ccccc6)c6ccccc6)cc4O5)c4cnn(-c5ccccc5)c43)cc2)cc1]

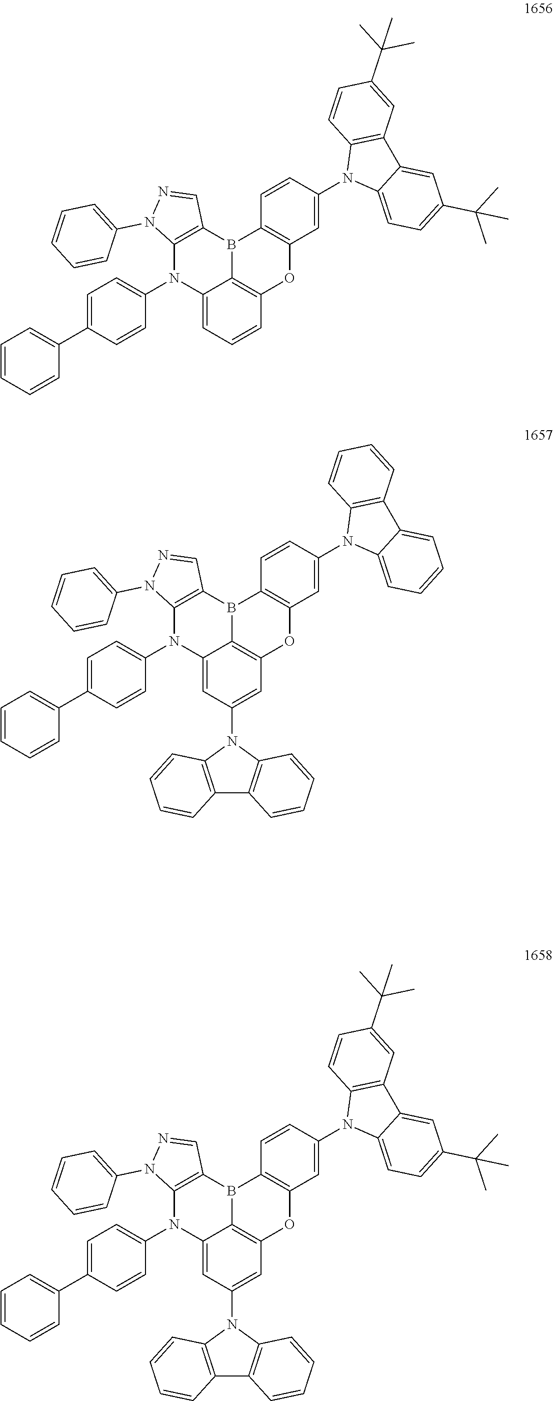 [SMILES: CC(C)(C)c1ccc2c(c1)c1cc(C(C)(C)C)ccc1n2-c1ccc2c(c1)Oc1cccc3c1B2c1cnn(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4cnn(-c5ccccc5)c43)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1cnn(-c2ccccc2)c1N3c1ccc(-c2ccccc2)cc1]

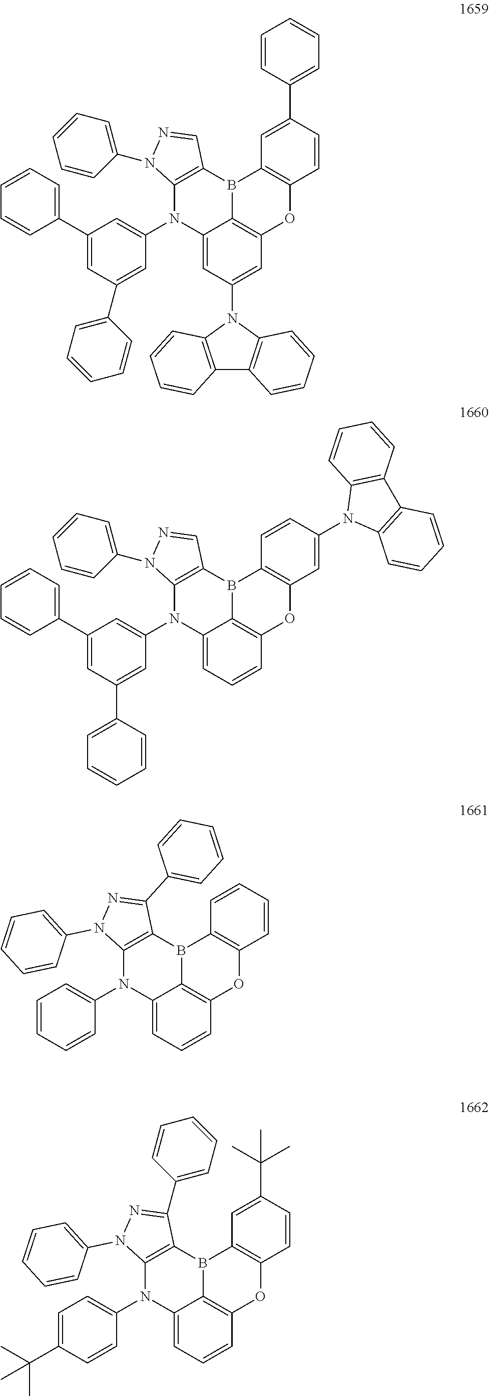 [SMILES: c1ccc(-c2cc(-c3ccccc3)cc(N3c4cc(-n5c6ccccc6c6ccccc65)cc5c4B(c4cc(-c6ccccc6)ccc4O5)c4cnn(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4cnn(-c5ccccc5)c43)c2)cc1 ; c1ccc(-c2nn(-c3ccccc3)c3c2B2c4ccccc4Oc4cccc(c42)N3c2ccccc2)cc1 ; CC(C)(C)c1ccc(N2c3cccc4c3B(c3cc(C(C)(C)C)ccc3O4)c3c(-c4ccccc4)nn(-c4ccccc4)c32)cc1]

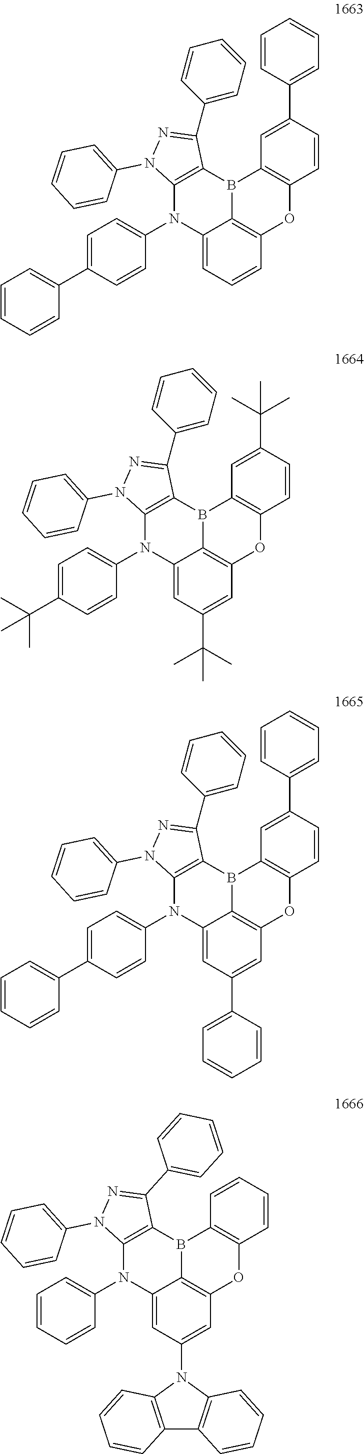 [SMILES: c1ccc(-c2ccc(N3c4cccc5c4B(c4cc(-c6ccccc6)ccc4O5)c4c(-c5ccccc5)nn(-c5ccccc5)c43)cc2)cc1 ; CC(C)(C)c1ccc(N2c3cc(C(C)(C)C)cc4c3B(c3cc(C(C)(C)C)ccc3O4)c3c(-c4ccccc4)nn(-c4ccccc4)c32)cc1 ; c1ccc(-c2ccc(N3c4cc(-c5ccccc5)cc5c4B(c4cc(-c6ccccc6)ccc4O5)c4c(-c5ccccc5)nn(-c5ccccc5)c43)cc2)cc1 ; c1ccc(-c2nn(-c3ccccc3)c3c2B2c4ccccc4Oc4cc(-n5c6ccccc6c6ccccc65)cc(c42)N3c2ccccc2)cc1]

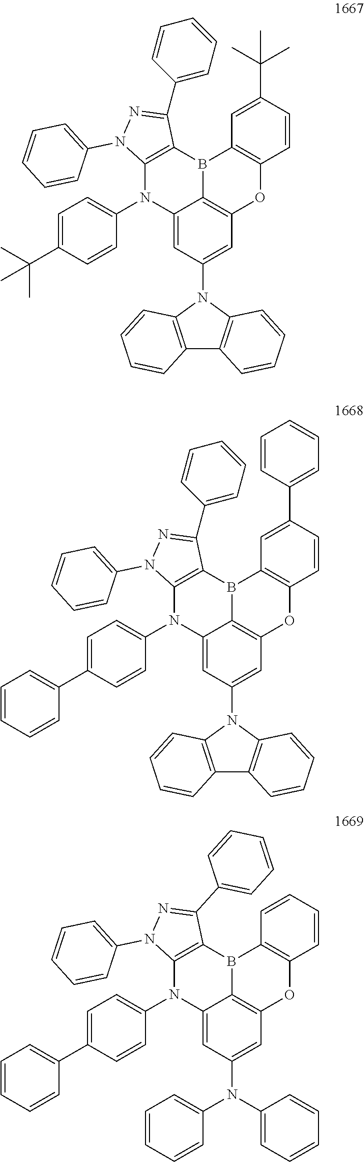

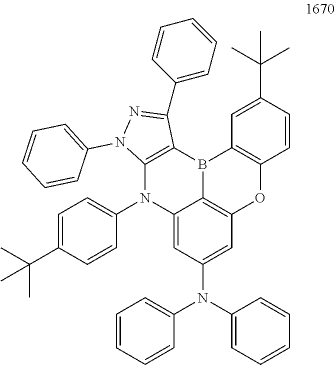 [SMILES: CC(C)(C)c1ccc(N2c3cc(N(c4ccccc4)c4ccccc4)cc4c3B(c3cc(C(C)(C)C)ccc3O4)c3c(-c4ccccc4)nn(-c4ccccc4)c32)cc1]

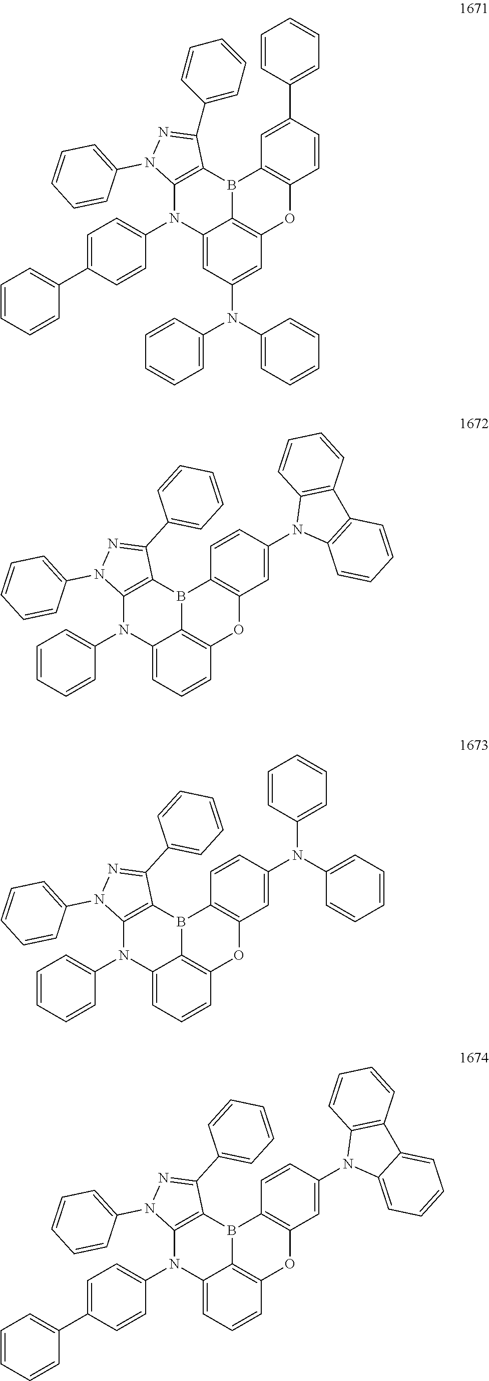 [SMILES: c1ccc(-c2ccc(N3c4cc(N(c5ccccc5)c5ccccc5)cc5c4B(c4cc(-c6ccccc6)ccc4O5)c4c(-c5ccccc5)nn(-c5ccccc5)c43)cc2)cc1 ; c1ccc(-c2nn(-c3ccccc3)c3c2B2c4ccc(-n5c6ccccc6c6ccccc65)cc4Oc4cccc(c42)N3c2ccccc2)cc1 ; c1ccc(-c2nn(-c3ccccc3)c3c2B2c4ccc(N(c5ccccc5)c5ccccc5)cc4Oc4cccc(c42)N3c2ccccc2)cc1 ; c1ccc(-c2ccc(N3c4cccc5c4B(c4ccc(-n6c7ccccc7c7ccccc76)cc4O5)c4c(-c5ccccc5)nn(-c5ccccc5)c43)cc2)cc1]

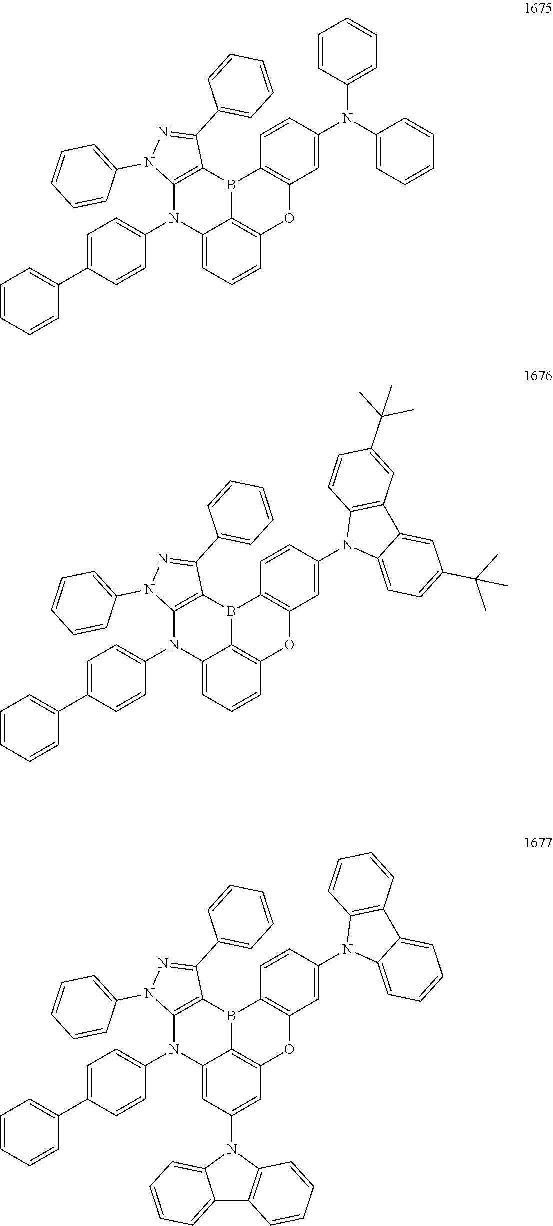 [SMILES: c1ccc(-c2ccc(N3c4cccc5c4B(c4ccc(N(c6ccccc6)c6ccccc6)cc4O5)c4c(-c5ccccc5)nn(-c5ccccc5)c43)cc2)cc1 ; CC(C)(C)c1ccc2c(c1)c1cc(C(C)(C)C)ccc1n2-c1ccc2c(c1)Oc1cccc3c1B2c1c(-c2ccccc2)nn(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4c(-c5ccccc5)nn(-c5ccccc5)c43)cc2)cc1]

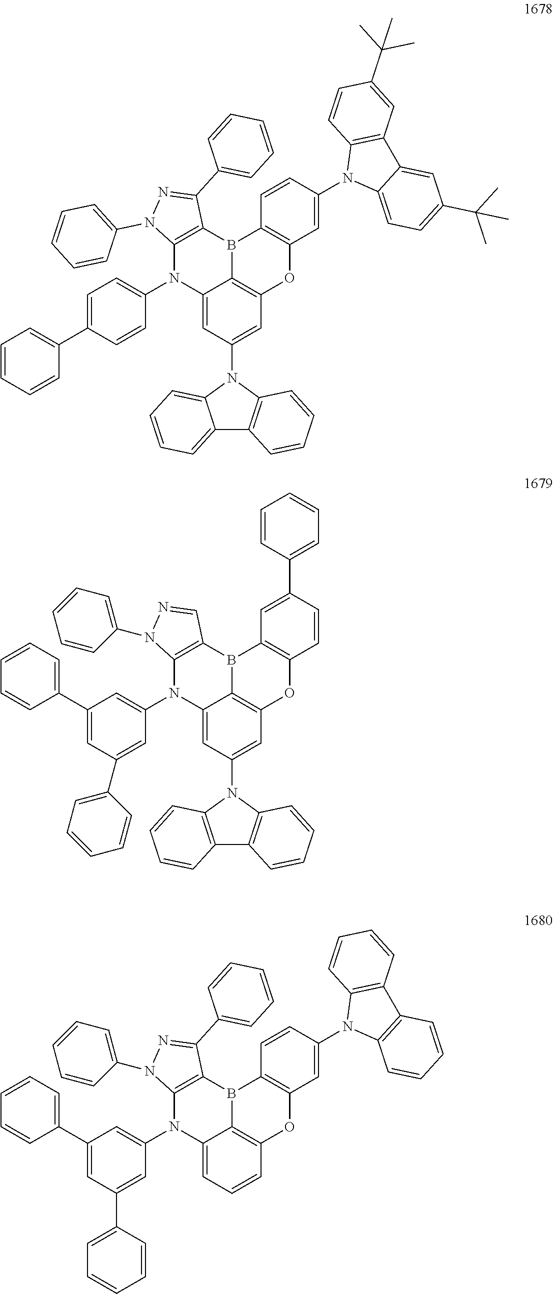

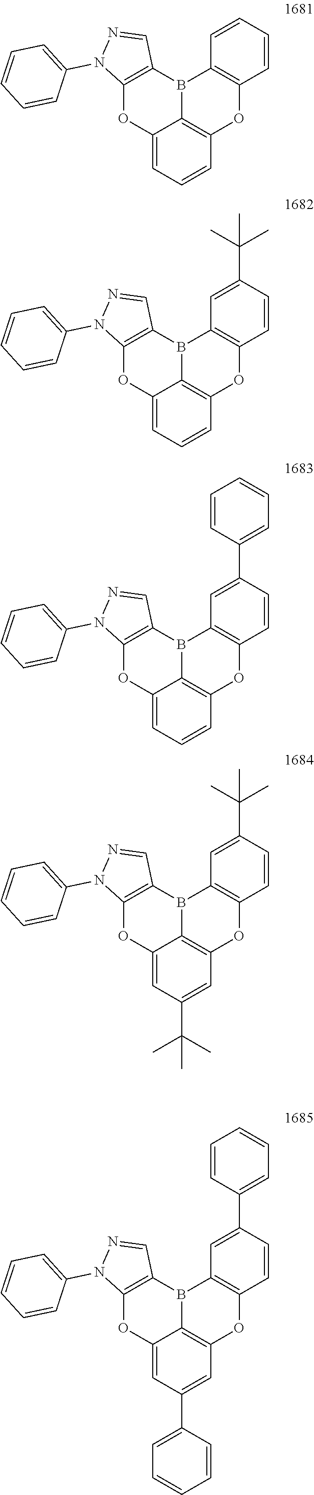 [SMILES: c1ccc(-n2ncc3c2Oc2cccc4c2B3c2ccccc2O4)cc1 ; CC(C)(C)c1ccc2c(c1)B1c3cnn(-c4ccccc4)c3Oc3cccc(c31)O2 ; c1ccc(-c2ccc3c(c2)B2c4cnn(-c5ccccc5)c4Oc4cccc(c42)O3)cc1 ; CC(C)(C)c1cc2c3c(c1)Oc1c(cnn1-c1ccccc1)B3c1cc(C(C)(C)C)ccc1O2 ; c1ccc(-c2cc3c4c(c2)Oc2c(cnn2-c2ccccc2)B4c2cc(-c4ccccc4)ccc2O3)cc1]

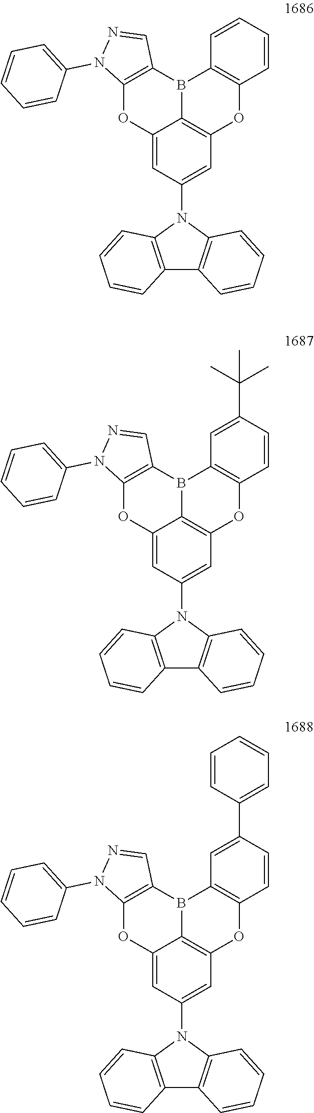 [SMILES: c1ccc(-n2ncc3c2Oc2cc(-n4c5ccccc5c5ccccc54)cc4c2B3c2ccccc2O4)cc1 ; CC(C)(C)c1ccc2c(c1)B1c3cnn(-c4ccccc4)c3Oc3cc(-n4c5ccccc5c5ccccc54)cc(c31)O2 ; c1ccc(-c2ccc3c(c2)B2c4cnn(-c5ccccc5)c4Oc4cc(-n5c6ccccc6c6ccccc65)cc(c42)O3)cc1]

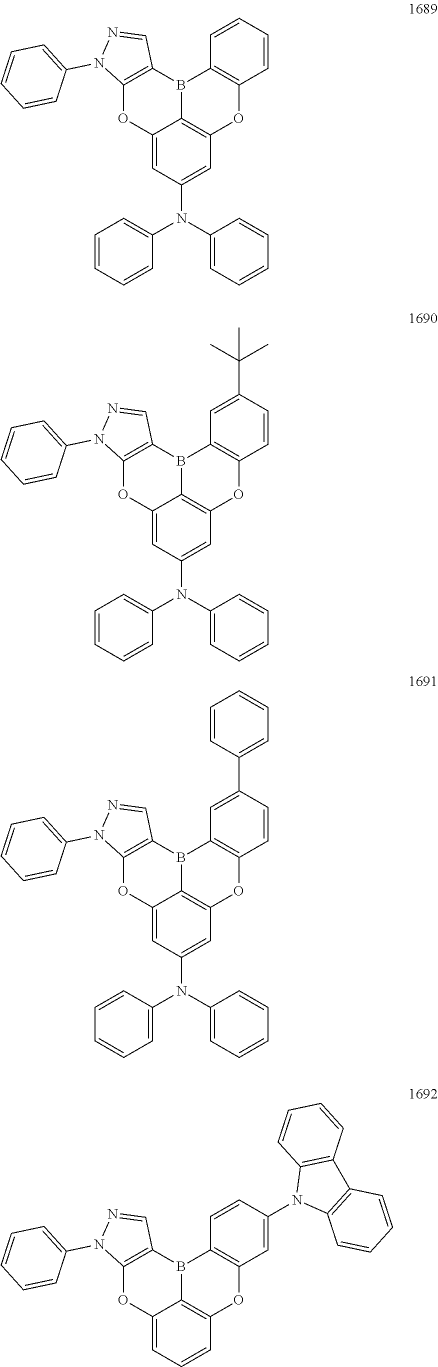

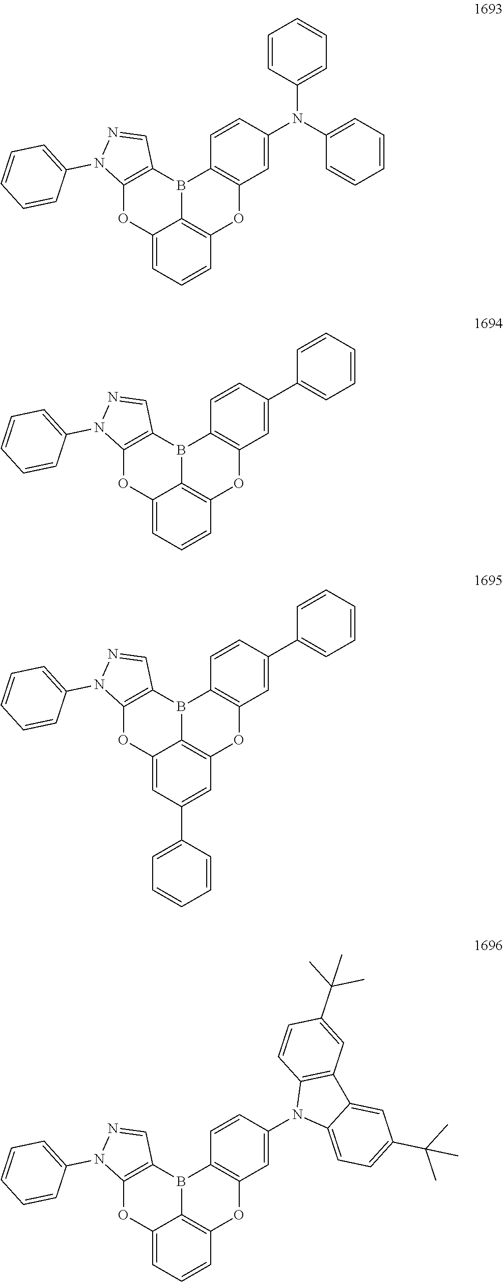 [SMILES: c1ccc(N(c2ccccc2)c2ccc3c(c2)Oc2cccc4c2B3c2cnn(-c3ccccc3)c2O4)cc1 ; c1ccc(-c2ccc3c(c2)Oc2cccc4c2B3c2cnn(-c3ccccc3)c2O4)cc1 ; c1ccc(-c2ccc3c(c2)Oc2cc(-c4ccccc4)cc4c2B3c2cnn(-c3ccccc3)c2O4)cc1 ; CC(C)(C)c1ccc2c(c1)c1cc(C(C)(C)C)ccc1n2-c1ccc2c(c1)Oc1cccc3c1B2c1cnn(-c2ccccc2)c1O3]

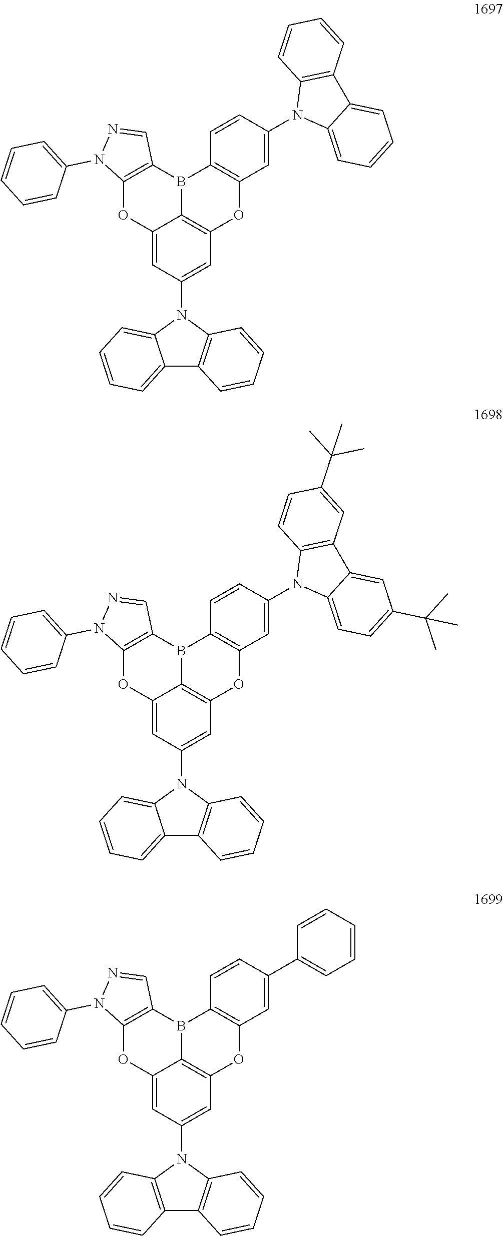 [SMILES: c1ccc(-n2ncc3c2Oc2cc(-n4c5ccccc5c5ccccc54)cc4c2B3c2ccc(-n3c5ccccc5c5ccccc53)cc2O4)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1cnn(-c2ccccc2)c1O3 ; c1ccc(-c2ccc3c(c2)Oc2cc(-n4c5ccccc5c5ccccc54)cc4c2B3c2cnn(-c3ccccc3)c2O4)cc1]

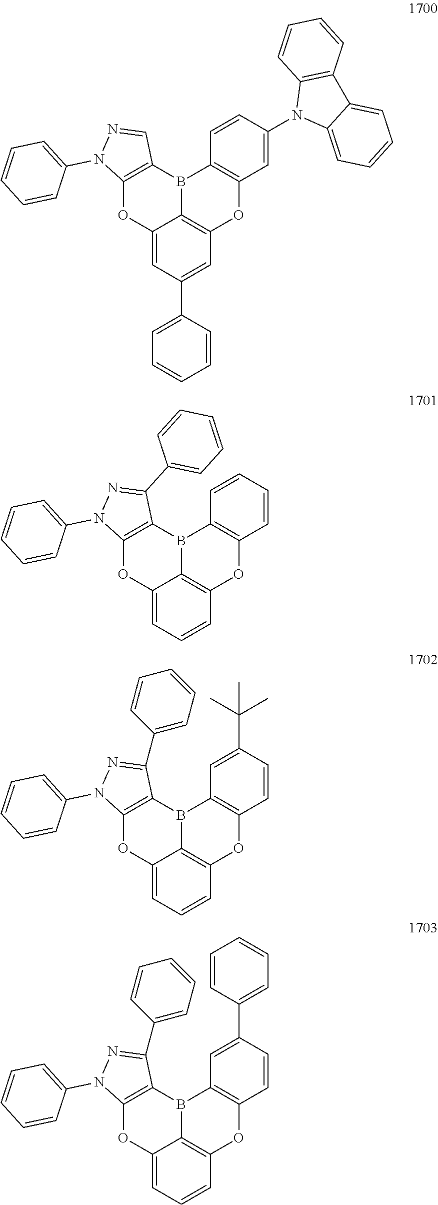

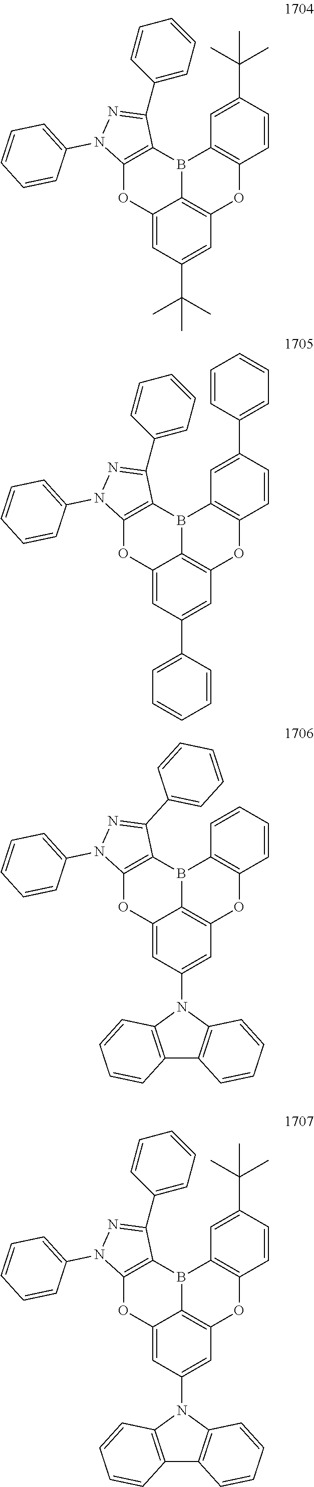

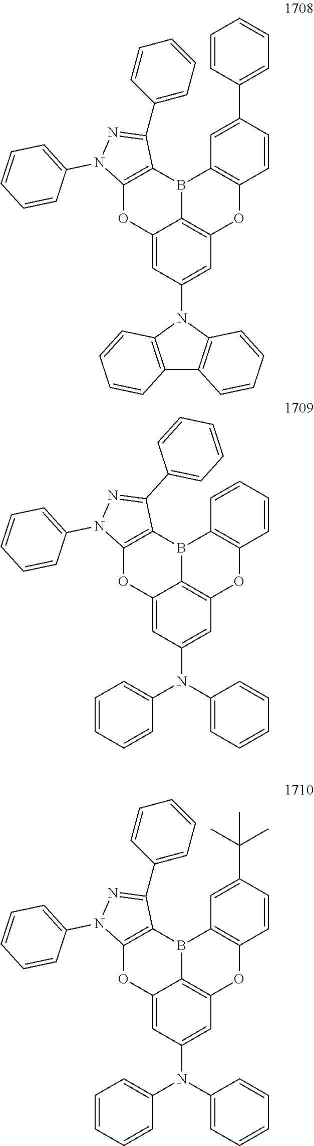

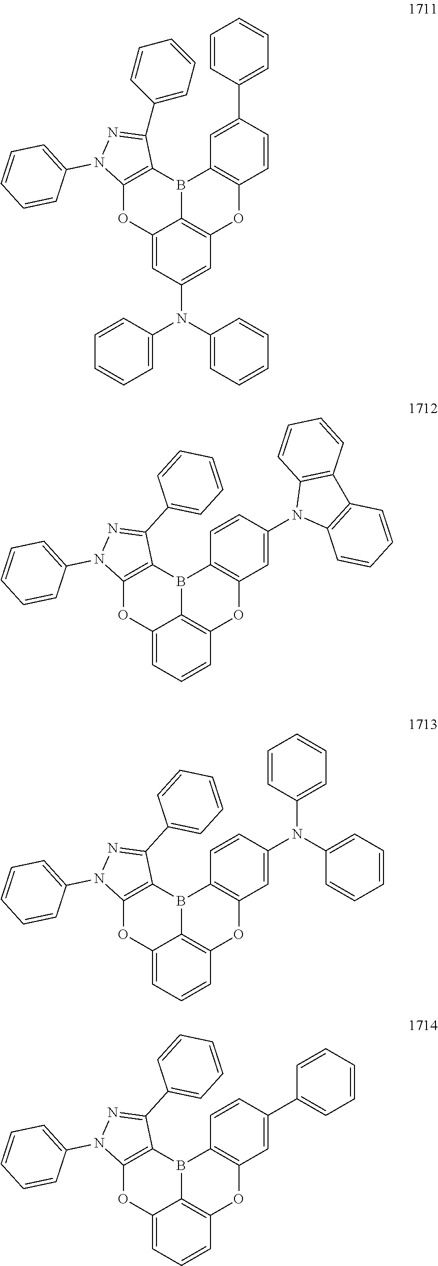

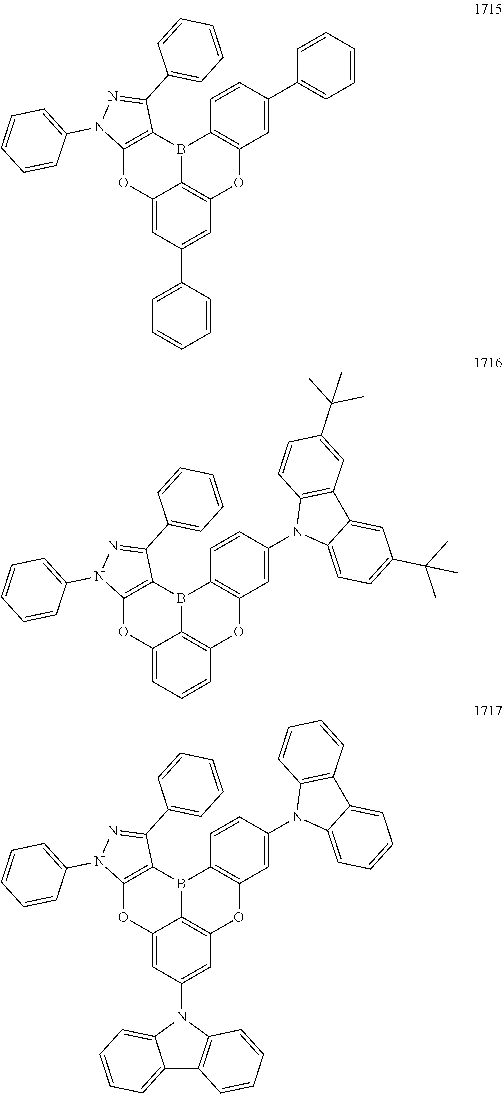 [SMILES: c1ccc(-c2ccc3c(c2)Oc2cc(-c4ccccc4)cc4c2B3c2c(-c3ccccc3)nn(-c3ccccc3)c2O4)cc1 ; CC(C)(C)c1ccc2c(c1)c1cc(C(C)(C)C)ccc1n2-c1ccc2c(c1)Oc1cccc3c1B2c1c(-c2ccccc2)nn(-c2ccccc2)c1O3 ; c1ccc(-c2nn(-c3ccccc3)c3c2B2c4ccc(-n5c6ccccc6c6ccccc65)cc4Oc4cc(-n5c6ccccc6c6ccccc65)cc(c42)O3)cc1]

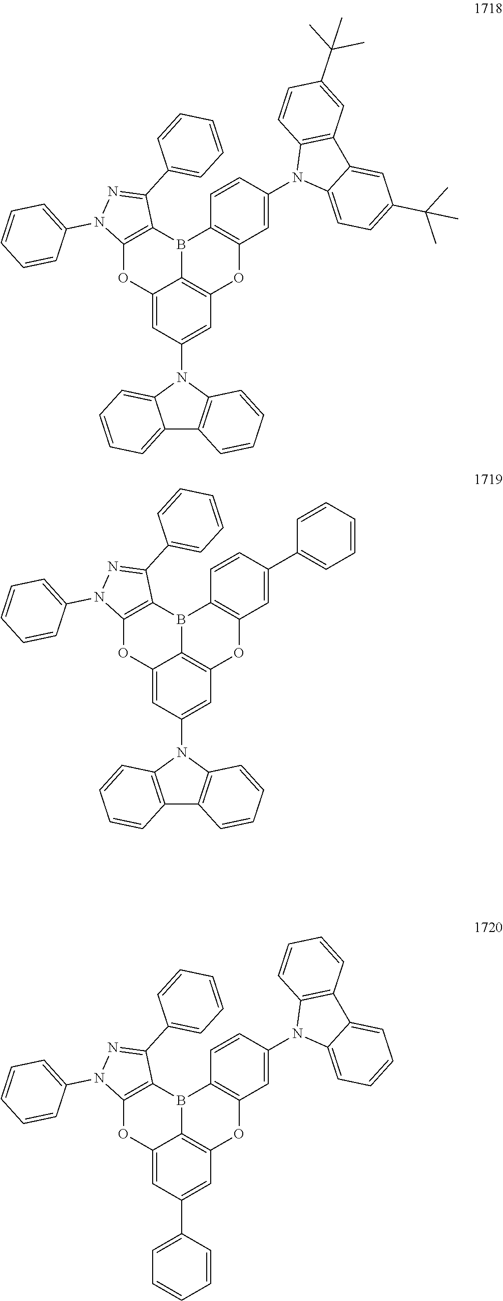

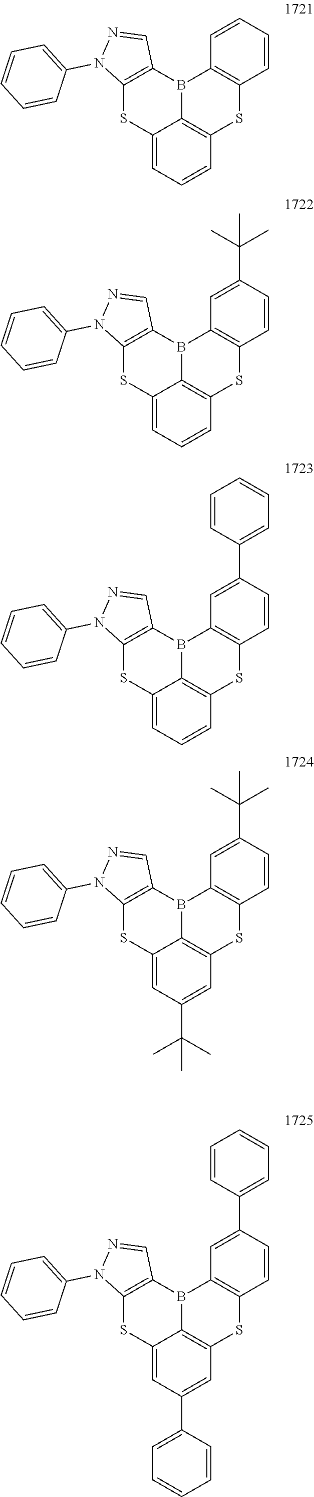 [SMILES: c1ccc(-n2ncc3c2Sc2cccc4c2B3c2ccccc2S4)cc1 ; CC(C)(C)c1ccc2c(c1)B1c3cnn(-c4ccccc4)c3Sc3cccc(c31)S2 ; c1ccc(-c2ccc3c(c2)B2c4cnn(-c5ccccc5)c4Sc4cccc(c42)S3)cc1 ; CC(C)(C)c1cc2c3c(c1)Sc1c(cnn1-c1ccccc1)B3c1cc(C(C)(C)C)ccc1S2 ; c1ccc(-c2cc3c4c(c2)Sc2c(cnn2-c2ccccc2)B4c2cc(-c4ccccc4)ccc2S3)cc1]

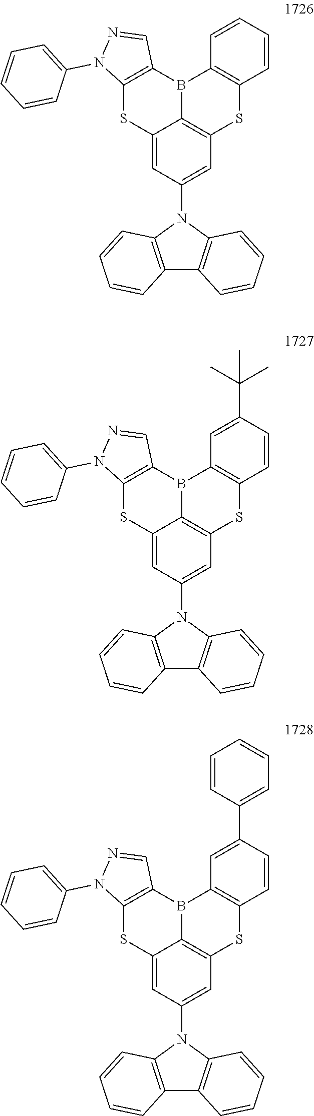 [SMILES: c1ccc(-n2ncc3c2Sc2cc(-n4c5ccccc5c5ccccc54)cc4c2B3c2ccccc2S4)cc1 ; CC(C)(C)c1ccc2c(c1)B1c3cnn(-c4ccccc4)c3Sc3cc(-n4c5ccccc5c5ccccc54)cc(c31)S2 ; c1ccc(-c2ccc3c(c2)B2c4cnn(-c5ccccc5)c4Sc4cc(-n5c6ccccc6c6ccccc65)cc(c42)S3)cc1]

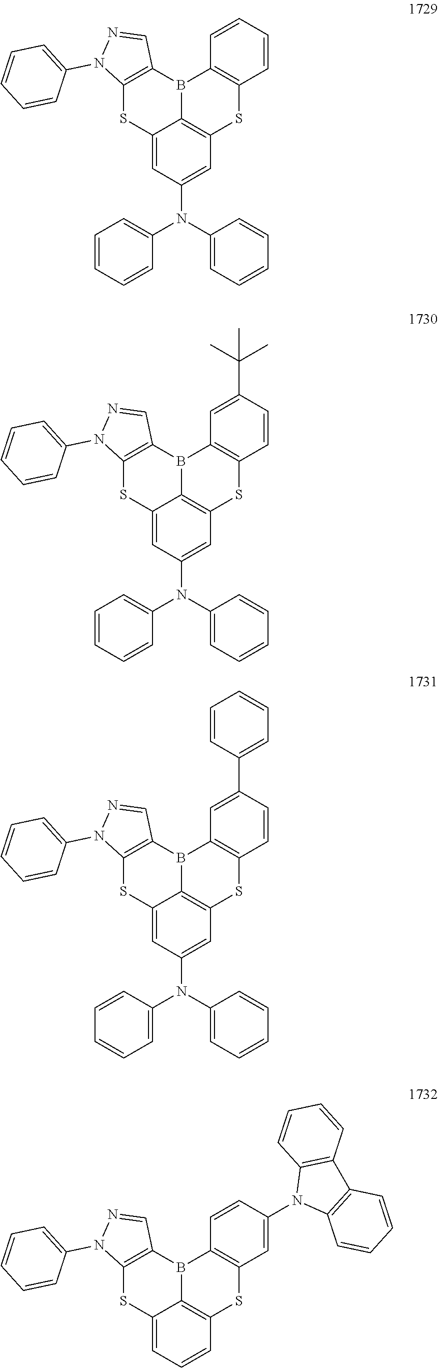

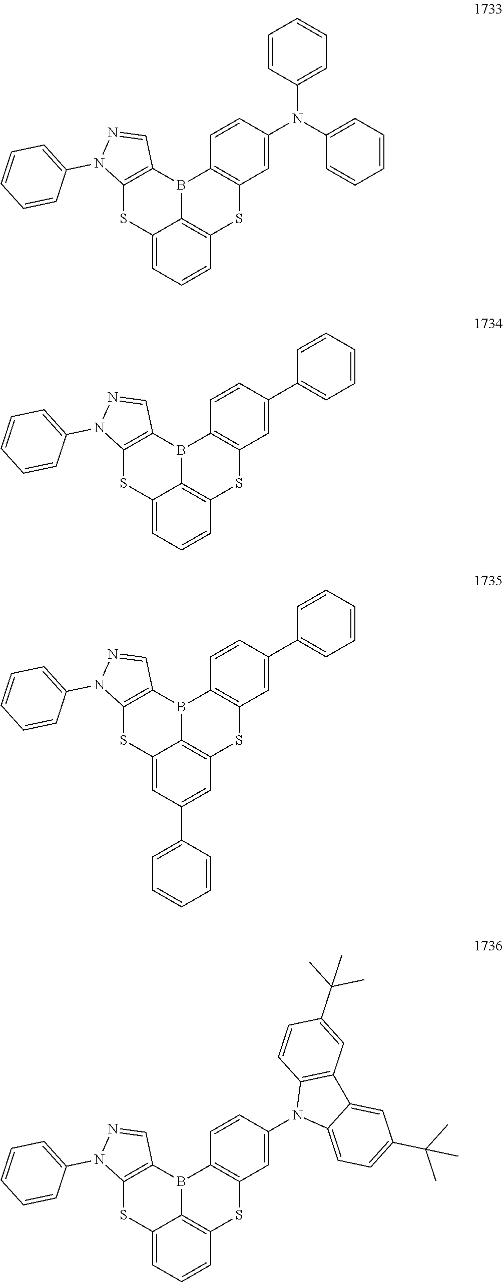 [SMILES: c1ccc(N(c2ccccc2)c2ccc3c(c2)Sc2cccc4c2B3c2cnn(-c3ccccc3)c2S4)cc1 ; c1ccc(-c2ccc3c(c2)Sc2cccc4c2B3c2cnn(-c3ccccc3)c2S4)cc1 ; c1ccc(-c2ccc3c(c2)Sc2cc(-c4ccccc4)cc4c2B3c2cnn(-c3ccccc3)c2S4)cc1 ; CC(C)(C)c1ccc2c(c1)c1cc(C(C)(C)C)ccc1n2-c1ccc2c(c1)Sc1cccc3c1B2c1cnn(-c2ccccc2)c1S3]

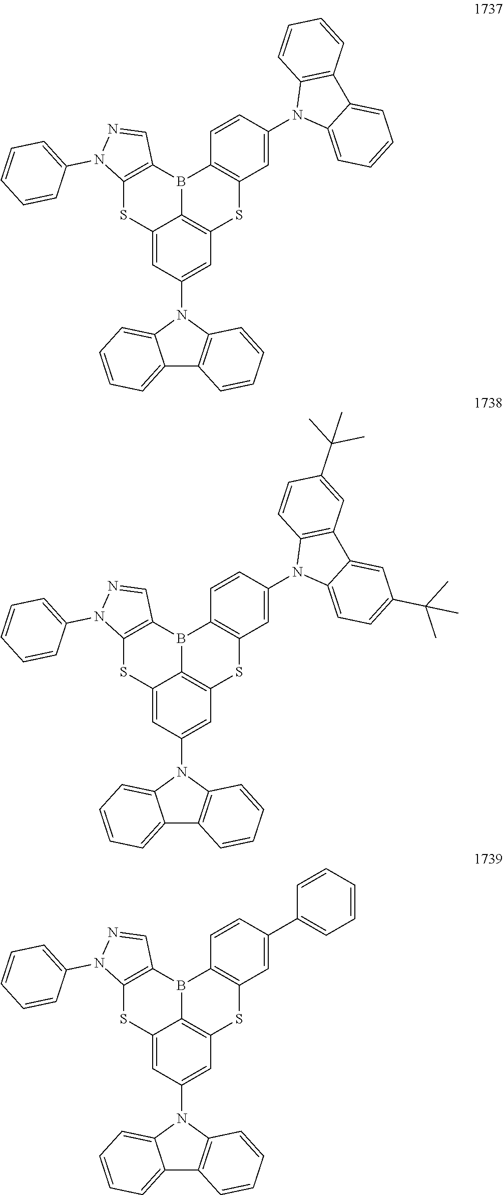 [SMILES: c1ccc(-n2ncc3c2Sc2cc(-n4c5ccccc5c5ccccc54)cc4c2B3c2ccc(-n3c5ccccc5c5ccccc53)cc2S4)cc1 ; CC(C)(C)c1ccc2c(c1)c1cc(C(C)(C)C)ccc1n2-c1ccc2c(c1)Sc1cc(-n3c4ccccc4c4ccccc43)cc3c1B2c1cnn(-c2ccccc2)c1S3 ; c1ccc(-c2ccc3c(c2)Sc2cc(-n4c5ccccc5c5ccccc54)cc4c2B3c2cnn(-c3ccccc3)c2S4)cc1]

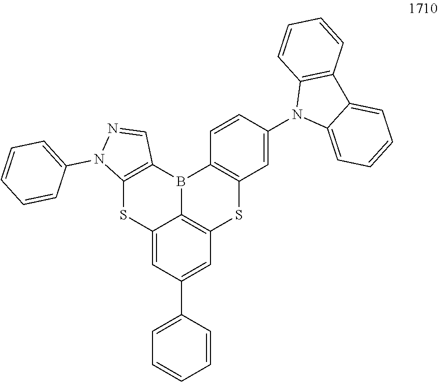 [SMILES: c1ccc(-c2cc3c4c(c2)Sc2c(cnn2-c2ccccc2)B4c2ccc(-n4c5ccccc5c5ccccc54)cc2S3)cc1]

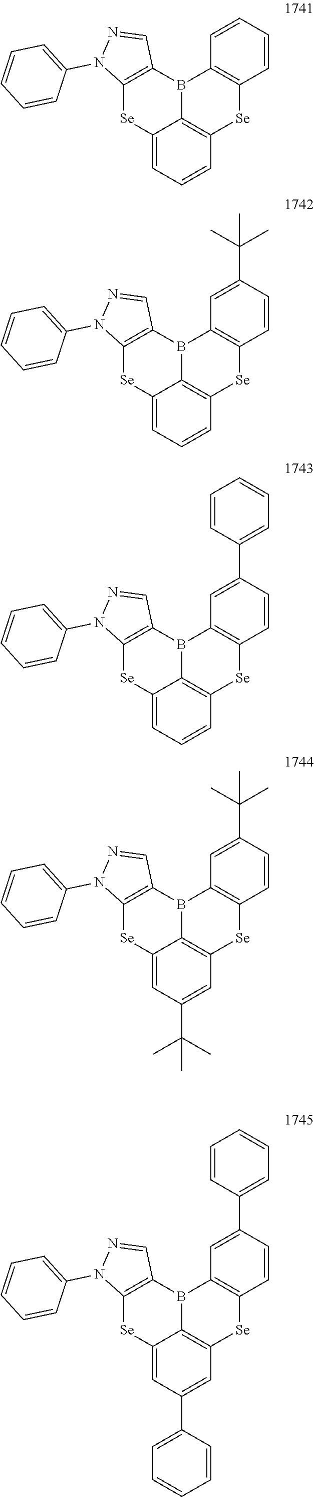 [SMILES: c1ccc(-n2ncc3c2[Se]c2cccc4c2B3c2ccccc2[Se]4)cc1 ; CC(C)(C)c1ccc2c(c1)B1c3cnn(-c4ccccc4)c3[Se]c3cccc(c31)[Se]2 ; c1ccc(-c2ccc3c(c2)B2c4cnn(-c5ccccc5)c4[Se]c4cccc(c42)[Se]3)cc1 ; CC(C)(C)c1cc2c3c(c1)[Se]c1c(cnn1-c1ccccc1)B3c1cc(C(C)(C)C)ccc1[Se]2 ; c1ccc(-c2cc3c4c(c2)[Se]c2c(cnn2-c2ccccc2)B4c2cc(-c4ccccc4)ccc2[Se]3)cc1]

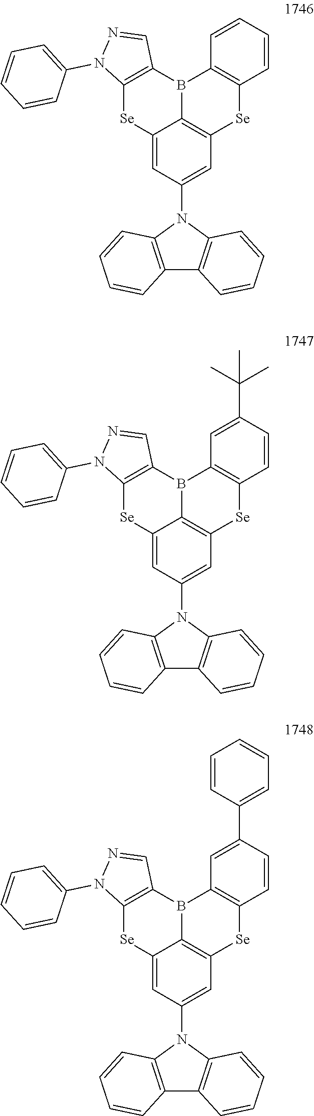 [SMILES: c1ccc(-n2ncc3c2[Se]c2cc(-n4c5ccccc5c5ccccc54)cc4c2B3c2ccccc2[Se]4)cc1 ; CC(C)(C)c1ccc2c(c1)B1c3cnn(-c4ccccc4)c3[Se]c3cc(-n4c5ccccc5c5ccccc54)cc(c31)[Se]2 ; c1ccc(-c2ccc3c(c2)B2c4cnn(-c5ccccc5)c4[Se]c4cc(-n5c6ccccc6c6ccccc65)cc(c42)[Se]3)cc1]

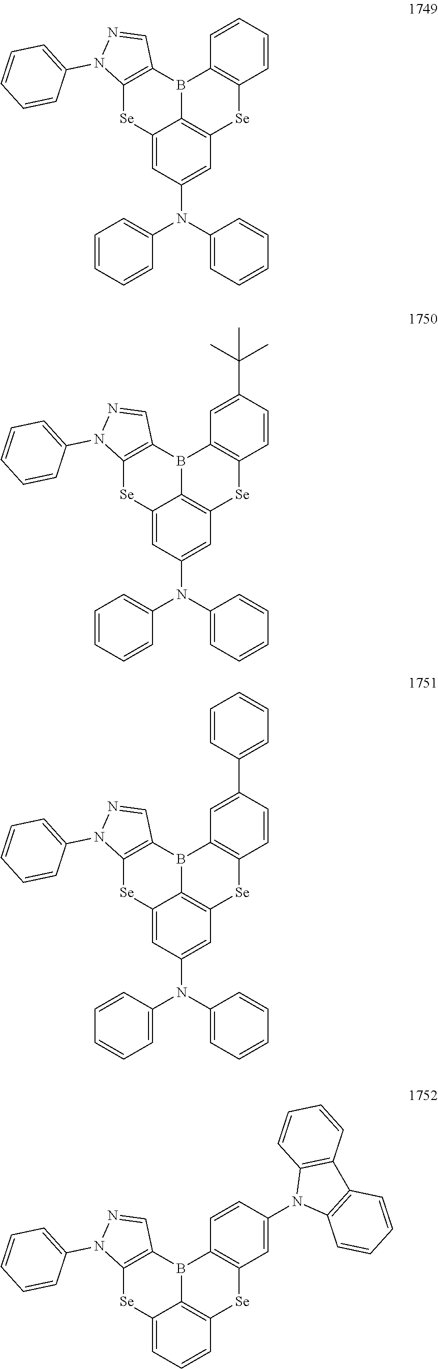

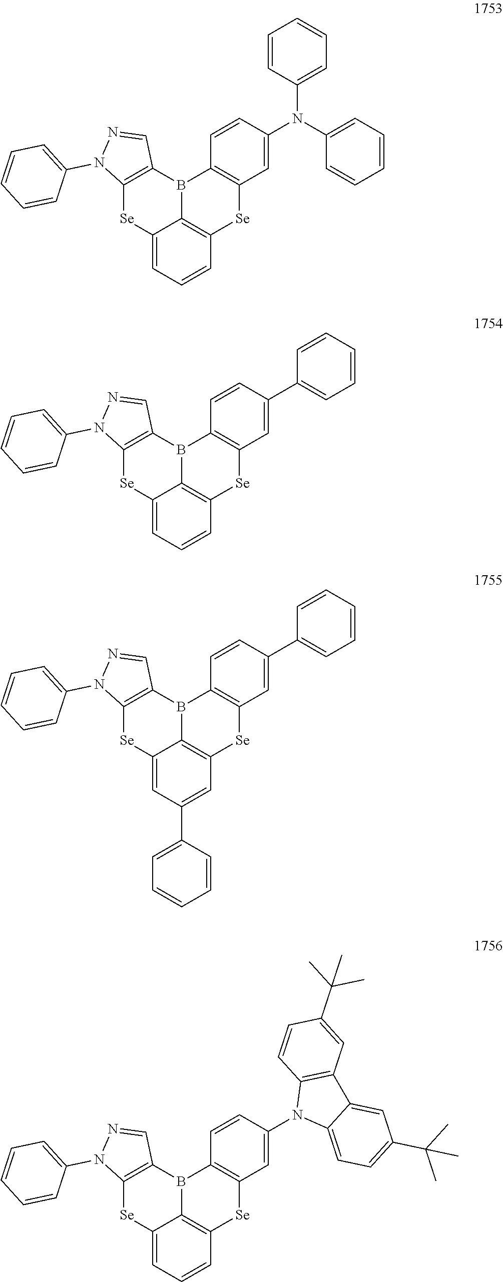 [SMILES: c1ccc(N(c2ccccc2)c2ccc3c(c2)[Se]c2cccc4c2B3c2cnn(-c3ccccc3)c2[Se]4)cc1 ; c1ccc(-c2ccc3c(c2)[Se]c2cccc4c2B3c2cnn(-c3ccccc3)c2[Se]4)cc1 ; c1ccc(-c2ccc3c(c2)[Se]c2cc(-c4ccccc4)cc4c2B3c2cnn(-c3ccccc3)c2[Se]4)cc1 ; CC(C)(C)c1ccc2c(c1)c1cc(C(C)(C)C)ccc1n2-c1ccc2c(c1)[Se]c1cccc3c1B2c1cnn(-c2ccccc2)c1[Se]3]

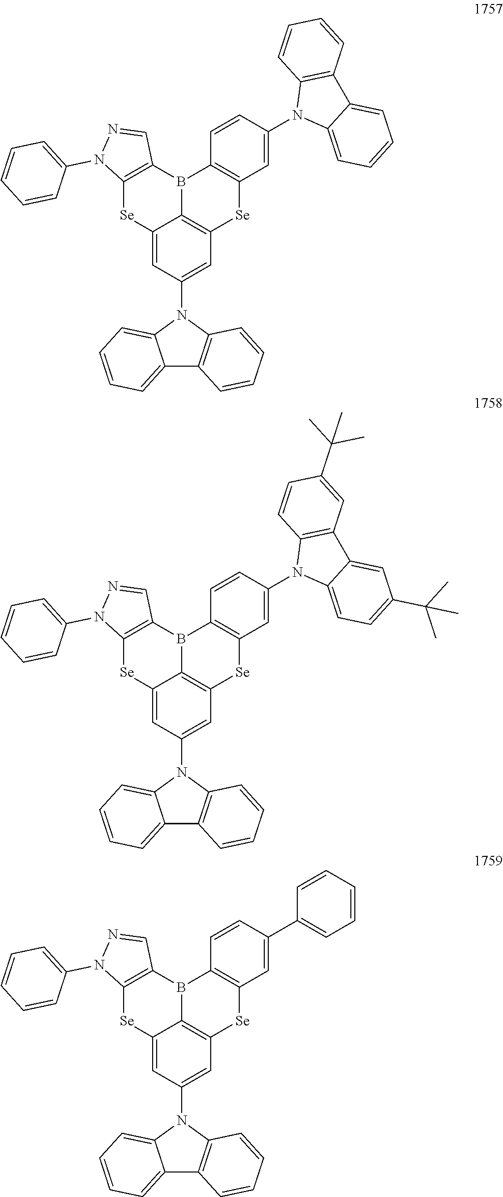 [SMILES: c1ccc(-n2ncc3c2[Se]c2cc(-n4c5ccccc5c5ccccc54)cc4c2B3c2ccc(-n3c5ccccc5c5ccccc53)cc2[Se]4)cc1 ; CC(C)(C)c1ccc2c(c1)c1cc(C(C)(C)C)ccc1n2-c1ccc2c(c1)[Se]c1cc(-n3c4ccccc4c4ccccc43)cc3c1B2c1cnn(-c2ccccc2)c1[Se]3 ; c1ccc(-c2ccc3c(c2)[Se]c2cc(-n4c5ccccc5c5ccccc54)cc4c2B3c2cnn(-c3ccccc3)c2[Se]4)cc1]

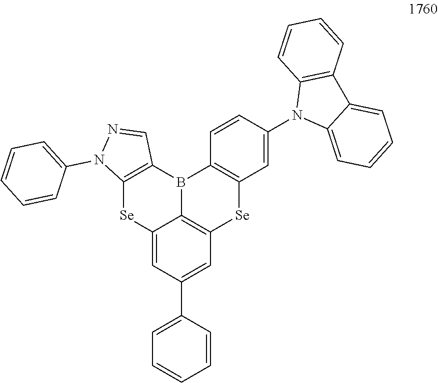 [SMILES: c1ccc(-c2cc3c4c(c2)[Se]c2c(cnn2-c2ccccc2)B4c2ccc(-n4c5ccccc5c5ccccc54)cc2[Se]3)cc1]

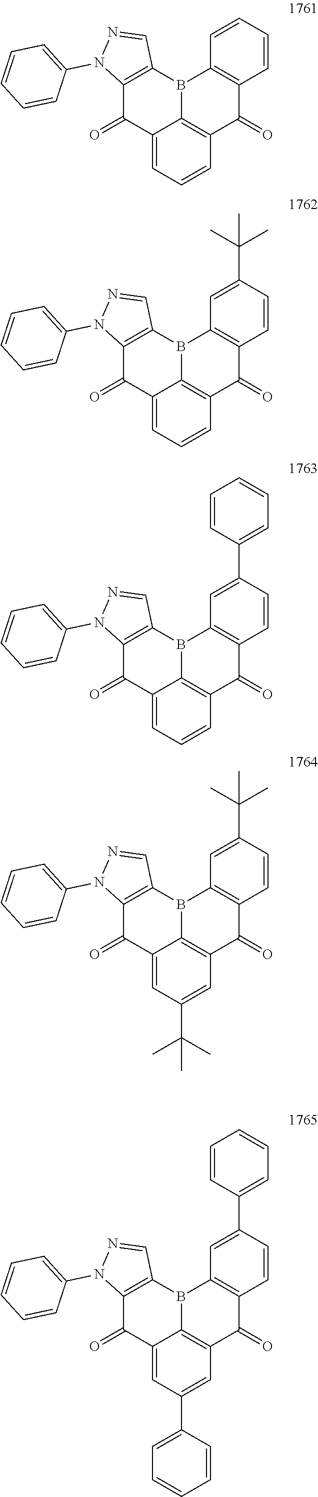 [SMILES: O=C1c2ccccc2B2c3cnn(-c4ccccc4)c3C(=O)c3cccc1c32 ; CC(C)(C)c1ccc2c(c1)B1c3cnn(-c4ccccc4)c3C(=O)c3cccc(c31)C2=O ; O=C1c2ccc(-c3ccccc3)cc2B2c3cnn(-c4ccccc4)c3C(=O)c3cccc1c32 ; CC(C)(C)c1ccc2c(c1)B1c3cnn(-c4ccccc4)c3C(=O)c3cc(C(C)(C)C)cc(c31)C2=O ; O=C1c2ccc(-c3ccccc3)cc2B2c3cnn(-c4ccccc4)c3C(=O)c3cc(-c4ccccc4)cc1c32]

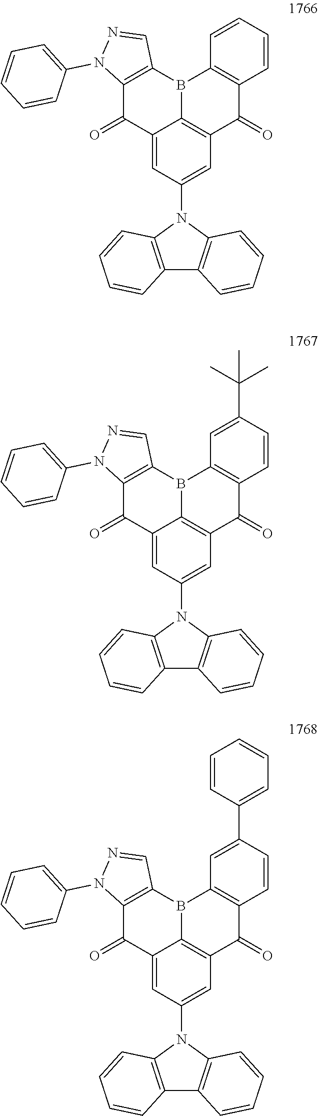 [SMILES: O=C1c2ccccc2B2c3cnn(-c4ccccc4)c3C(=O)c3cc(-n4c5ccccc5c5ccccc54)cc1c32 ; CC(C)(C)c1ccc2c(c1)B1c3cnn(-c4ccccc4)c3C(=O)c3cc(-n4c5ccccc5c5ccccc54)cc(c31)C2=O ; O=C1c2ccc(-c3ccccc3)cc2B2c3cnn(-c4ccccc4)c3C(=O)c3cc(-n4c5ccccc5c5ccccc54)cc1c32]

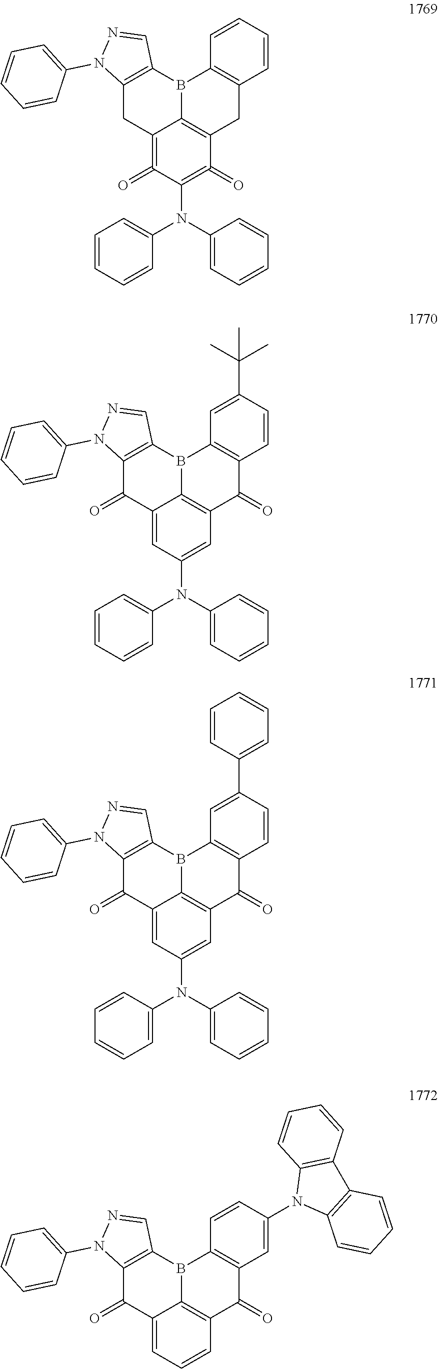

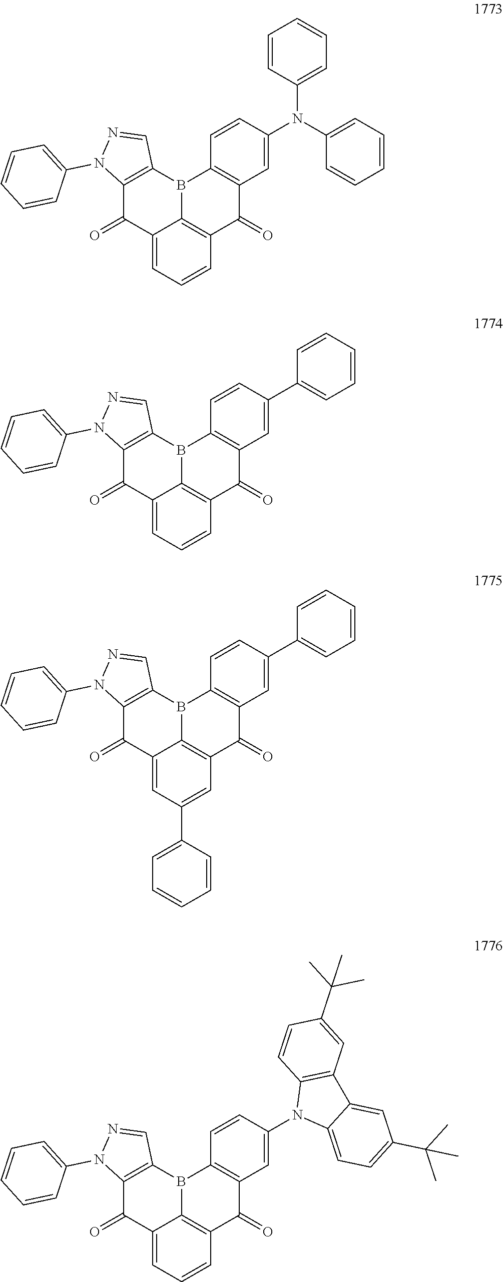 [SMILES: O=C1c2cc(N(c3ccccc3)c3ccccc3)ccc2B2c3cnn(-c4ccccc4)c3C(=O)c3cccc1c32 ; O=C1c2cc(-c3ccccc3)ccc2B2c3cnn(-c4ccccc4)c3C(=O)c3cccc1c32 ; O=C1c2cc(-c3ccccc3)ccc2B2c3cnn(-c4ccccc4)c3C(=O)c3cc(-c4ccccc4)cc1c32 ; CC(C)(C)c1ccc2c(c1)c1cc(C(C)(C)C)ccc1n2-c1ccc2c(c1)C(=O)c1cccc3c1B2c1cnn(-c2ccccc2)c1C3=O]

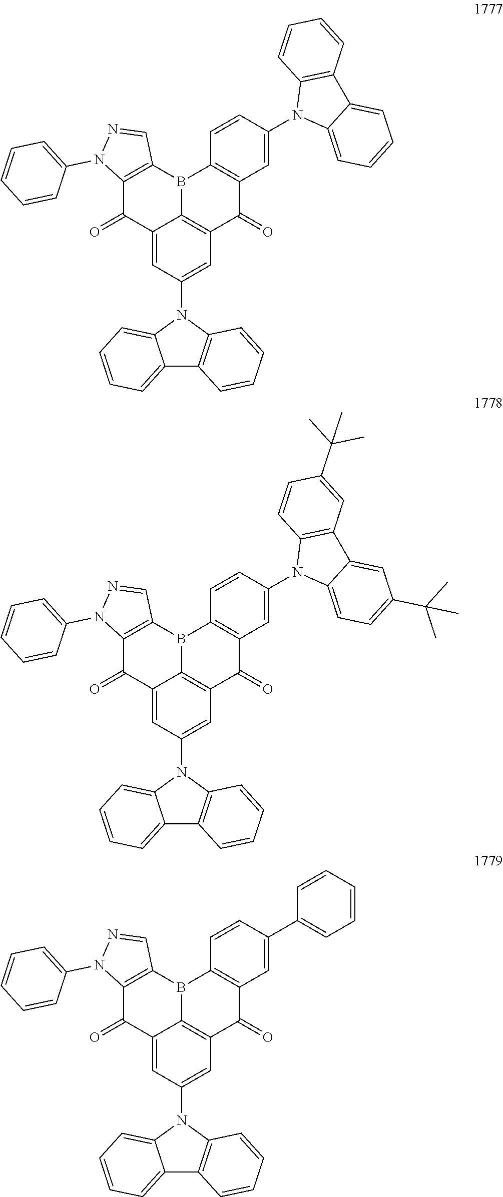 [SMILES: O=C1c2cc(-n3c4ccccc4c4ccccc43)ccc2B2c3cnn(-c4ccccc4)c3C(=O)c3cc(-n4c5ccccc5c5ccccc54)cc1c32 ; CC(C)(C)c1ccc2c(c1)c1cc(C(C)(C)C)ccc1n2-c1ccc2c(c1)C(=O)c1cc(-n3c4ccccc4c4ccccc43)cc3c1B2c1cnn(-c2ccccc2)c1C3=O ; O=C1c2cc(-c3ccccc3)ccc2B2c3cnn(-c4ccccc4)c3C(=O)c3cc(-n4c5ccccc5c5ccccc54)cc1c32]

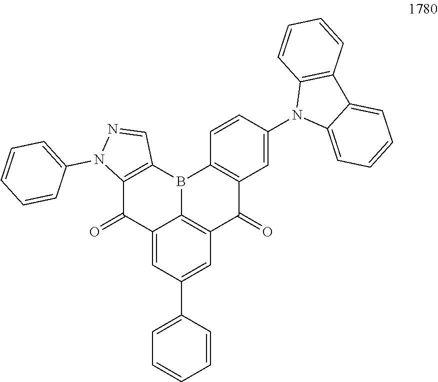 [SMILES: O=C1c2cc(-n3c4ccccc4c4ccccc43)ccc2B2c3cnn(-c4ccccc4)c3C(=O)c3cc(-c4ccccc4)cc1c32]

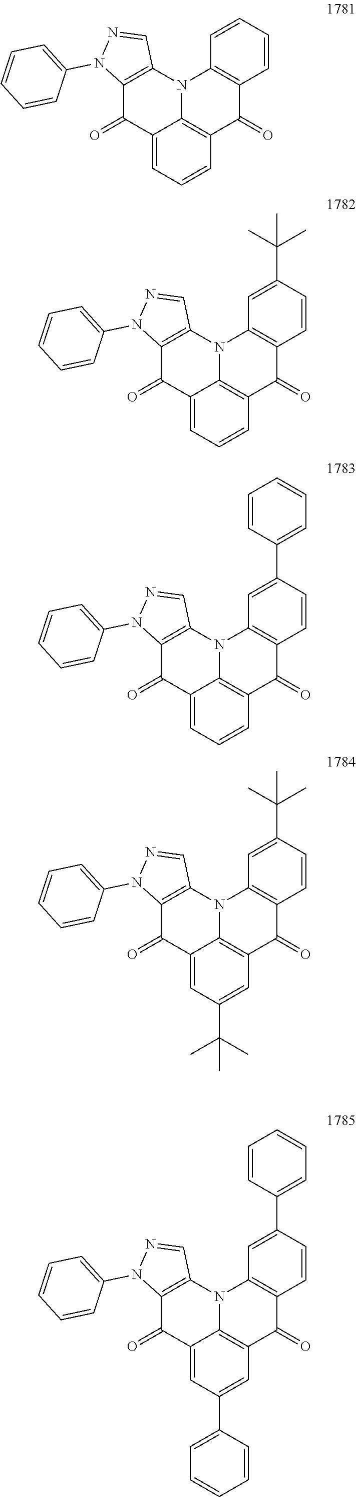 [SMILES: O=c1c2ccccc2n2c3cnn(-c4ccccc4)c3c(=O)c3cccc1c32 ; CC(C)(C)c1ccc2c(=O)c3cccc4c(=O)c5c(cnn5-c5ccccc5)n(c2c1)c34 ; O=c1c2ccc(-c3ccccc3)cc2n2c3cnn(-c4ccccc4)c3c(=O)c3cccc1c32 ; CC(C)(C)c1cc2c(=O)c3ccc(C(C)(C)C)cc3n3c4cnn(-c5ccccc5)c4c(=O)c(c1)c23 ; O=c1c2ccc(-c3ccccc3)cc2n2c3cnn(-c4ccccc4)c3c(=O)c3cc(-c4ccccc4)cc1c32]

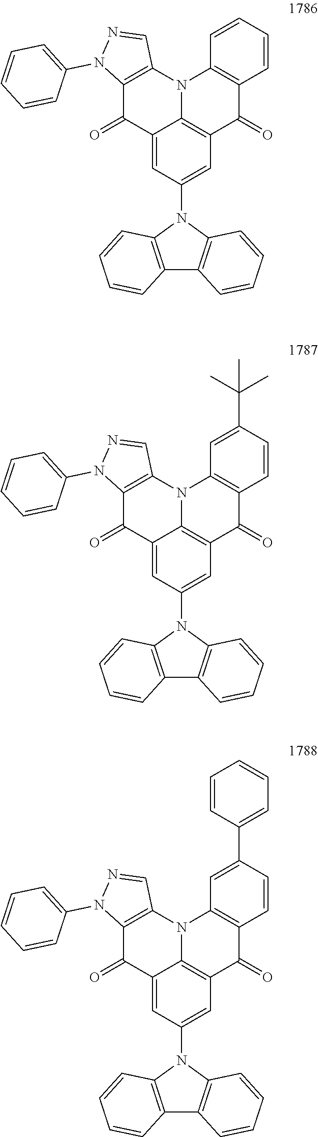 [SMILES: O=c1c2ccccc2n2c3cnn(-c4ccccc4)c3c(=O)c3cc(-n4c5ccccc5c5ccccc54)cc1c32 ; CC(C)(C)c1ccc2c(=O)c3cc(-n4c5ccccc5c5ccccc54)cc4c(=O)c5c(cnn5-c5ccccc5)n(c2c1)c34 ; O=c1c2ccc(-c3ccccc3)cc2n2c3cnn(-c4ccccc4)c3c(=O)c3cc(-n4c5ccccc5c5ccccc54)cc1c32]

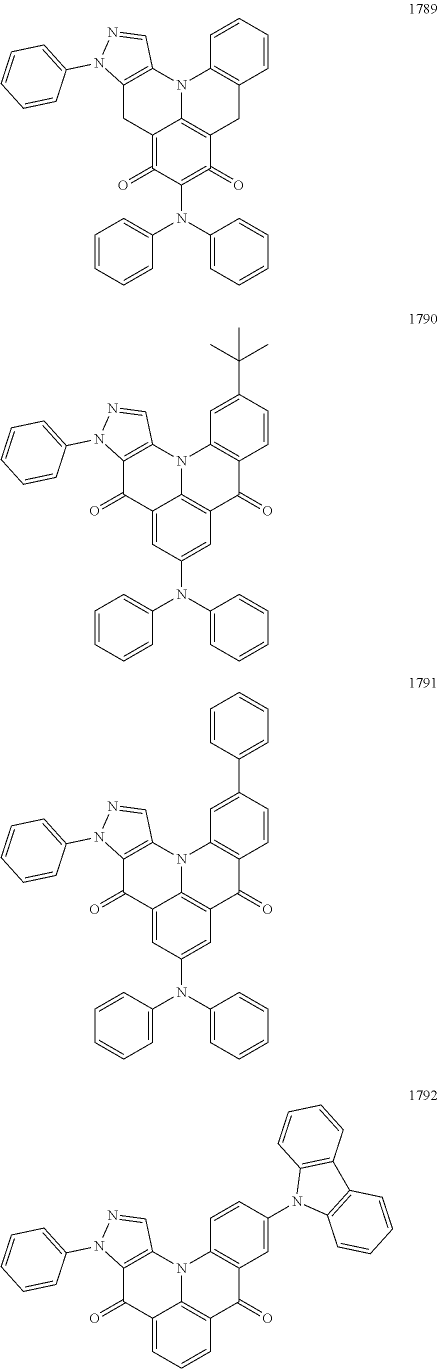

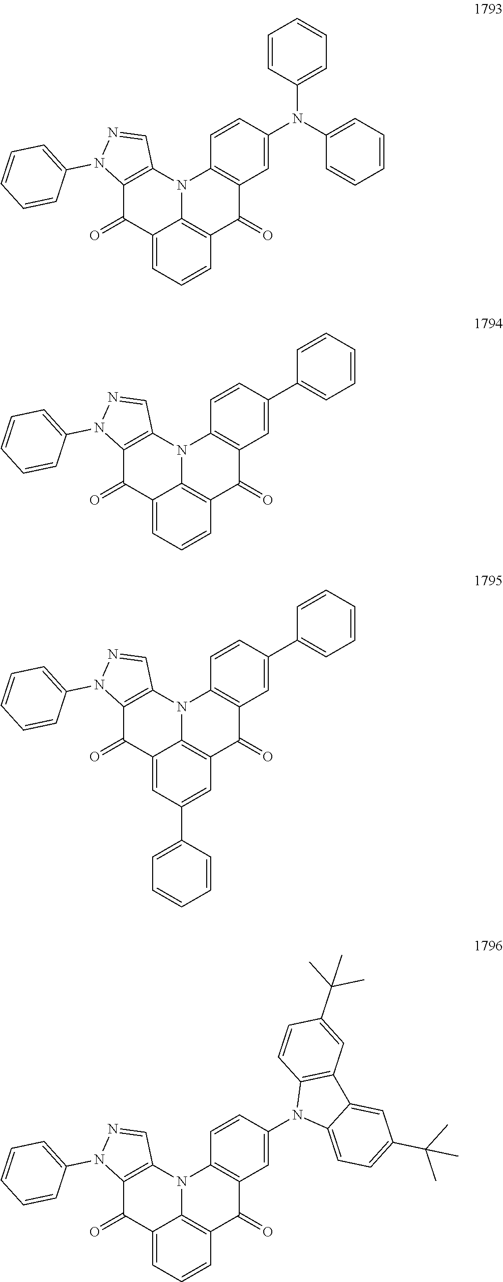 [SMILES: O=c1c2cc(N(c3ccccc3)c3ccccc3)ccc2n2c3cnn(-c4ccccc4)c3c(=O)c3cccc1c32 ; O=c1c2cc(-c3ccccc3)ccc2n2c3cnn(-c4ccccc4)c3c(=O)c3cccc1c32 ; O=c1c2cc(-c3ccccc3)ccc2n2c3cnn(-c4ccccc4)c3c(=O)c3cc(-c4ccccc4)cc1c32 ; CC(C)(C)c1ccc2c(c1)c1cc(C(C)(C)C)ccc1n2-c1ccc2c(c1)c(=O)c1cccc3c(=O)c4c(cnn4-c4ccccc4)n2c13]

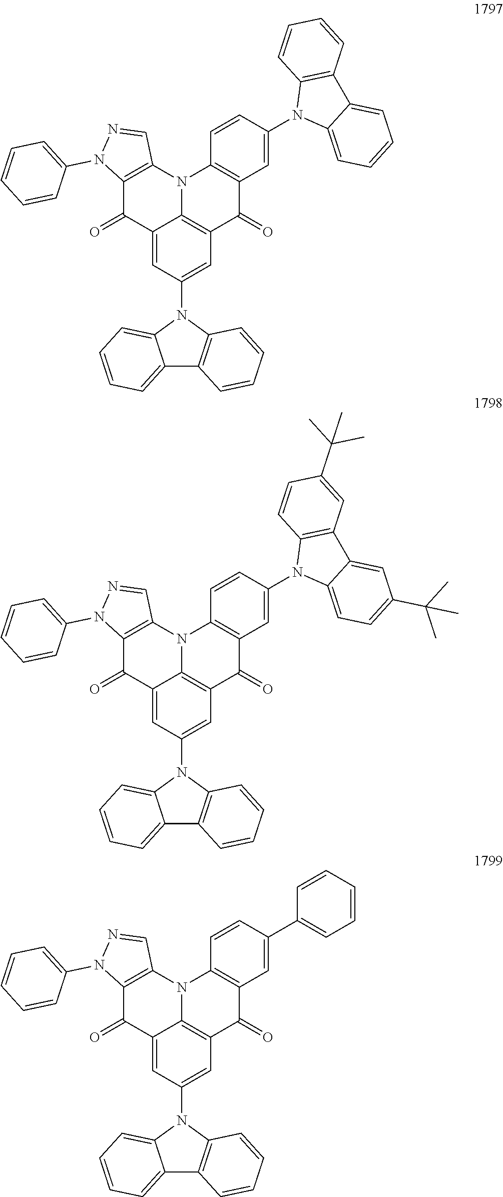 [SMILES: O=c1c2cc(-n3c4ccccc4c4ccccc43)ccc2n2c3cnn(-c4ccccc4)c3c(=O)c3cc(-n4c5ccccc5c5ccccc54)cc1c32 ; CC(C)(C)c1ccc2c(c1)c1cc(C(C)(C)C)ccc1n2-c1ccc2c(c1)c(=O)c1cc(-n3c4ccccc4c4ccccc43)cc3c(=O)c4c(cnn4-c4ccccc4)n2c13 ; O=c1c2cc(-c3ccccc3)ccc2n2c3cnn(-c4ccccc4)c3c(=O)c3cc(-n4c5ccccc5c5ccccc54)cc1c32]

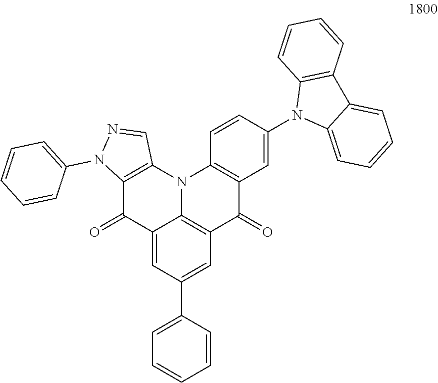 [SMILES: O=c1c2cc(-n3c4ccccc4c4ccccc43)ccc2n2c3cnn(-c4ccccc4)c3c(=O)c3cc(-c4ccccc4)cc1c32]

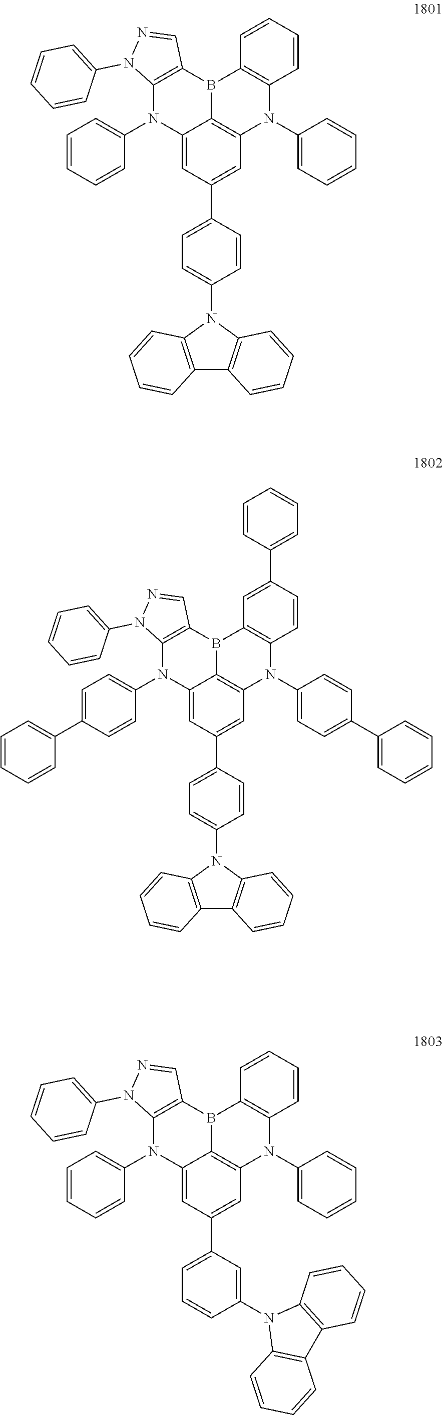 [SMILES: c1ccc(N2c3ccccc3B3c4cnn(-c5ccccc5)c4N(c4ccccc4)c4cc(-c5ccc(-n6c7ccccc7c7ccccc76)cc5)cc2c43)cc1 ; c1ccc(-c2ccc(N3c4ccc(-c5ccccc5)cc4B4c5cnn(-c6ccccc6)c5N(c5ccc(-c6ccccc6)cc5)c5cc(-c6ccc(-n7c8ccccc8c8ccccc87)cc6)cc3c54)cc2)cc1 ; c1ccc(N2c3ccccc3B3c4cnn(-c5ccccc5)c4N(c4ccccc4)c4cc(-c5cccc(-n6c7ccccc7c7ccccc76)c5)cc2c43)cc1]

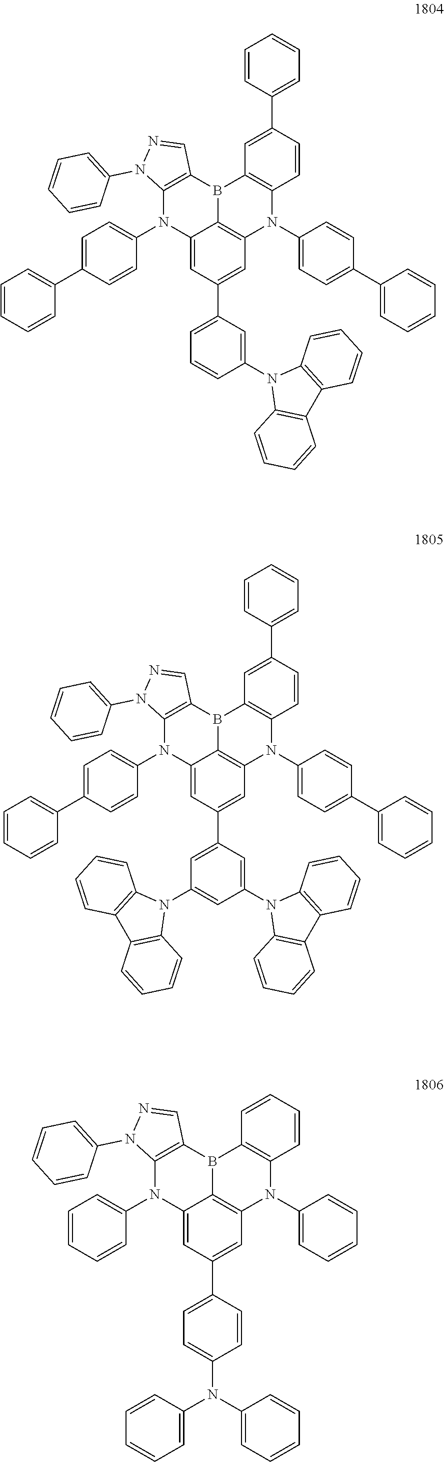 [SMILES: c1ccc(-c2ccc(N3c4ccc(-c5ccccc5)cc4B4c5cnn(-c6ccccc6)c5N(c5ccc(-c6ccccc6)cc5)c5cc(-c6cccc(-n7c8ccccc8c8ccccc87)c6)cc3c54)cc2)cc1 ; c1ccc(-c2ccc(N3c4ccc(-c5ccccc5)cc4B4c5cnn(-c6ccccc6)c5N(c5ccc(-c6ccccc6)cc5)c5cc(-c6cc(-n7c8ccccc8c8ccccc87)cc(-n7c8ccccc8c8ccccc87)c6)cc3c54)cc2)cc1 ; c1ccc(N(c2ccccc2)c2ccc(-c3cc4c5c(c3)N(c3ccccc3)c3c(cnn3-c3ccccc3)B5c3ccccc3N4c3ccccc3)cc2)cc1]

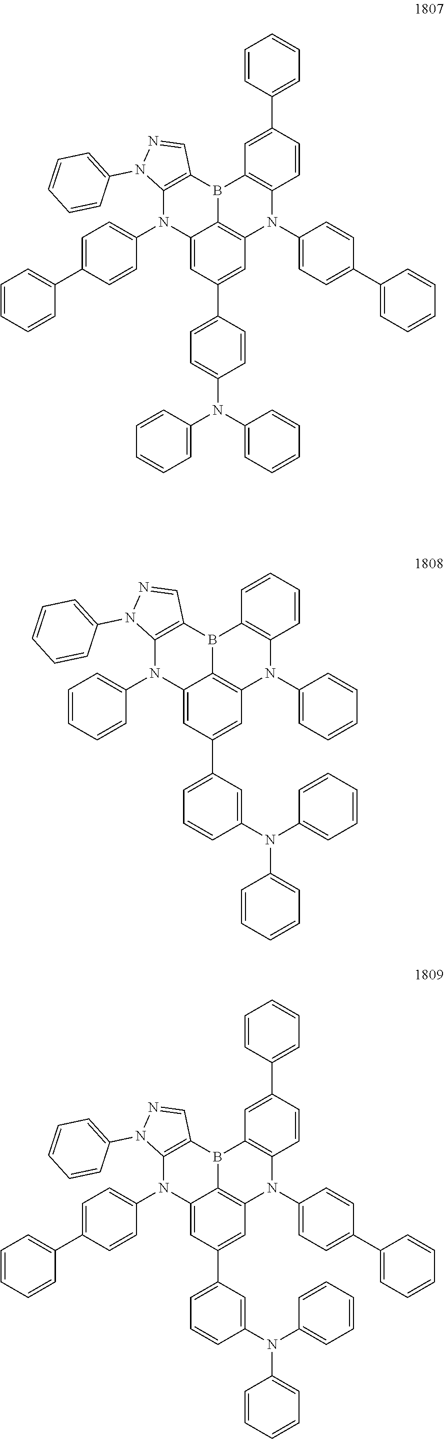 [SMILES: c1ccc(-c2ccc(N3c4ccc(-c5ccccc5)cc4B4c5cnn(-c6ccccc6)c5N(c5ccc(-c6ccccc6)cc5)c5cc(-c6ccc(N(c7ccccc7)c7ccccc7)cc6)cc3c54)cc2)cc1 ; c1ccc(N(c2ccccc2)c2cccc(-c3cc4c5c(c3)N(c3ccccc3)c3c(cnn3-c3ccccc3)B5c3ccccc3N4c3ccccc3)c2)cc1 ; c1ccc(-c2ccc(N3c4ccc(-c5ccccc5)cc4B4c5cnn(-c6ccccc6)c5N(c5ccc(-c6ccccc6)cc5)c5cc(-c6cccc(N(c7ccccc7)c7ccccc7)c6)cc3c54)cc2)cc1]

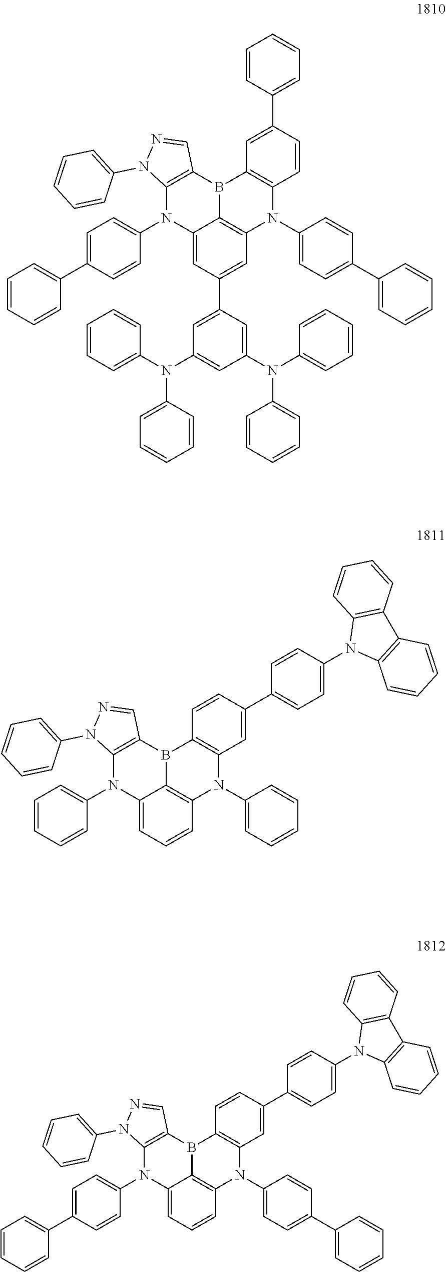 [SMILES: c1ccc(-c2ccc(N3c4ccc(-c5ccccc5)cc4B4c5cnn(-c6ccccc6)c5N(c5ccc(-c6ccccc6)cc5)c5cc(-c6cc(N(c7ccccc7)c7ccccc7)cc(N(c7ccccc7)c7ccccc7)c6)cc3c54)cc2)cc1 ; c1ccc(N2c3cc(-c4ccc(-n5c6ccccc6c6ccccc65)cc4)ccc3B3c4cnn(-c5ccccc5)c4N(c4ccccc4)c4cccc2c43)cc1 ; c1ccc(-c2ccc(N3c4cc(-c5ccc(-n6c7ccccc7c7ccccc76)cc5)ccc4B4c5cnn(-c6ccccc6)c5N(c5ccc(-c6ccccc6)cc5)c5cccc3c54)cc2)cc1]

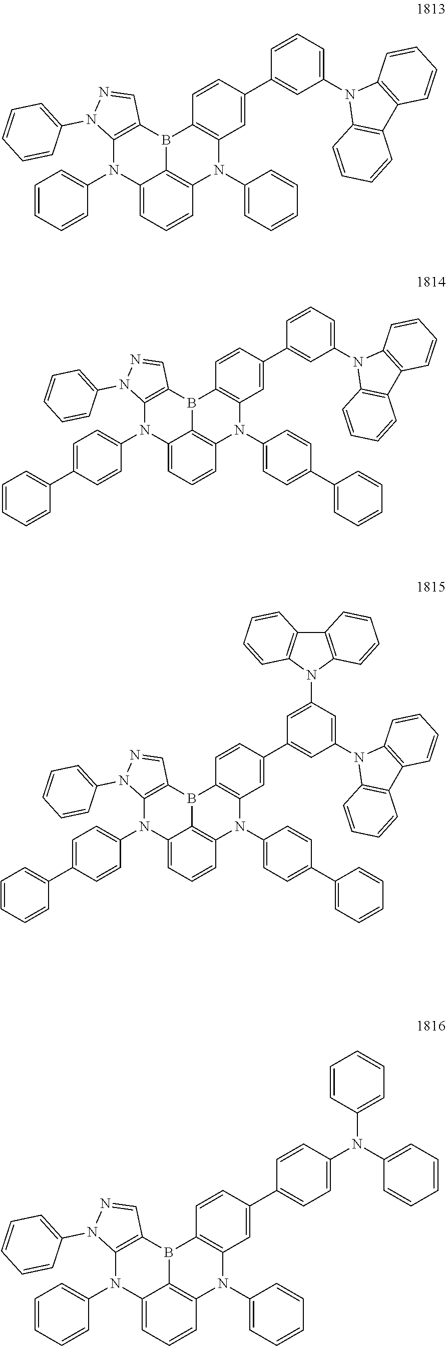 [SMILES: c1ccc(N2c3cc(-c4cccc(-n5c6ccccc6c6ccccc65)c4)ccc3B3c4cnn(-c5ccccc5)c4N(c4ccccc4)c4cccc2c43)cc1 ; c1ccc(-c2ccc(N3c4cc(-c5cccc(-n6c7ccccc7c7ccccc76)c5)ccc4B4c5cnn(-c6ccccc6)c5N(c5ccc(-c6ccccc6)cc5)c5cccc3c54)cc2)cc1 ; c1ccc(-c2ccc(N3c4cc(-c5cc(-n6c7ccccc7c7ccccc76)cc(-n6c7ccccc7c7ccccc76)c5)ccc4B4c5cnn(-c6ccccc6)c5N(c5ccc(-c6ccccc6)cc5)c5cccc3c54)cc2)cc1 ; c1ccc(N(c2ccccc2)c2ccc(-c3ccc4c(c3)N(c3ccccc3)c3cccc5c3B4c3cnn(-c4ccccc4)c3N5c3ccccc3)cc2)cc1]

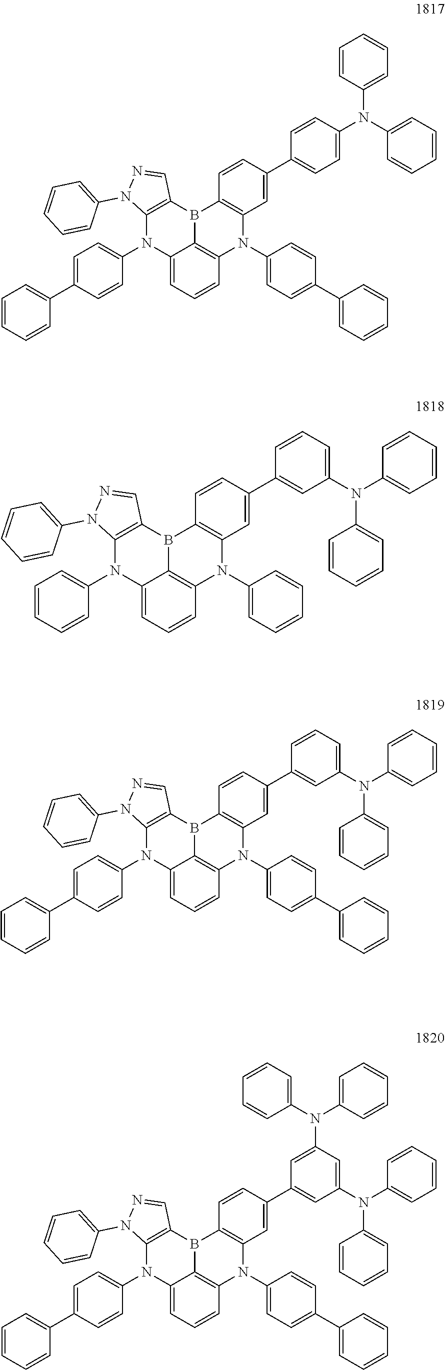 [SMILES: c1ccc(-c2ccc(N3c4cc(-c5ccc(N(c6ccccc6)c6ccccc6)cc5)ccc4B4c5cnn(-c6ccccc6)c5N(c5ccc(-c6ccccc6)cc5)c5cccc3c54)cc2)cc1 ; c1ccc(N(c2ccccc2)c2cccc(-c3ccc4c(c3)N(c3ccccc3)c3cccc5c3B4c3cnn(-c4ccccc4)c3N5c3ccccc3)c2)cc1 ; c1ccc(-c2ccc(N3c4cc(-c5cccc(N(c6ccccc6)c6ccccc6)c5)ccc4B4c5cnn(-c6ccccc6)c5N(c5ccc(-c6ccccc6)cc5)c5cccc3c54)cc2)cc1 ; c1ccc(-c2ccc(N3c4cc(-c5cc(N(c6ccccc6)c6ccccc6)cc(N(c6ccccc6)c6ccccc6)c5)ccc4B4c5cnn(-c6ccccc6)c5N(c5ccc(-c6ccccc6)cc5)c5cccc3c54)cc2)cc1]

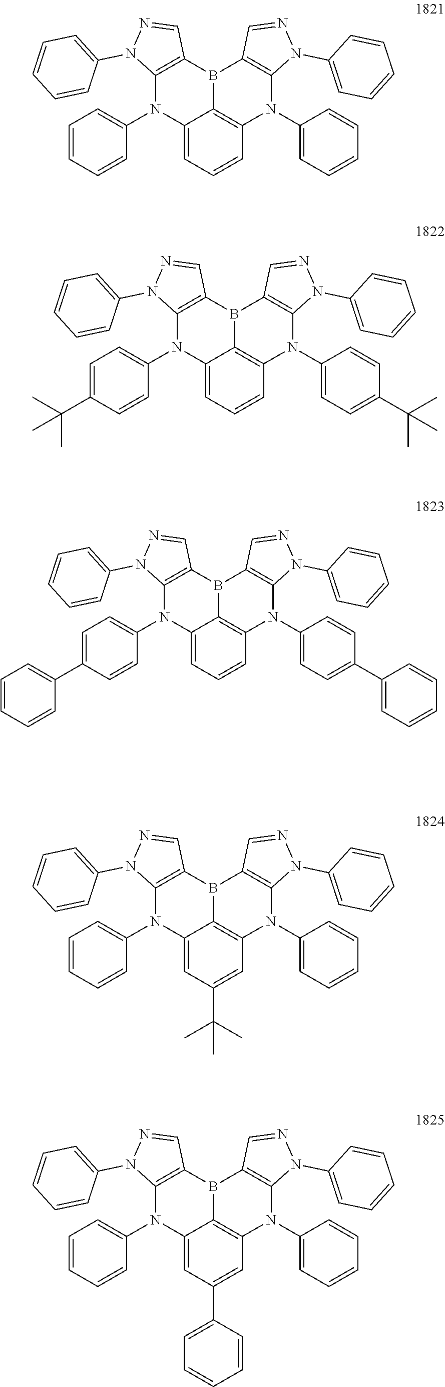 [SMILES: c1ccc(N2c3cccc4c3B(c3cnn(-c5ccccc5)c32)c2cnn(-c3ccccc3)c2N4c2ccccc2)cc1 ; CC(C)(C)c1ccc(N2c3cccc4c3B(c3cnn(-c5ccccc5)c32)c2cnn(-c3ccccc3)c2N4c2ccc(C(C)(C)C)cc2)cc1 ; c1ccc(-c2ccc(N3c4cccc5c4B(c4cnn(-c6ccccc6)c43)c3cnn(-c4ccccc4)c3N5c3ccc(-c4ccccc4)cc3)cc2)cc1 ; CC(C)(C)c1cc2c3c(c1)N(c1ccccc1)c1c(cnn1-c1ccccc1)B3c1cnn(-c3ccccc3)c1N2c1ccccc1 ; c1ccc(-c2cc3c4c(c2)N(c2ccccc2)c2c(cnn2-c2ccccc2)B4c2cnn(-c4ccccc4)c2N3c2ccccc2)cc1]

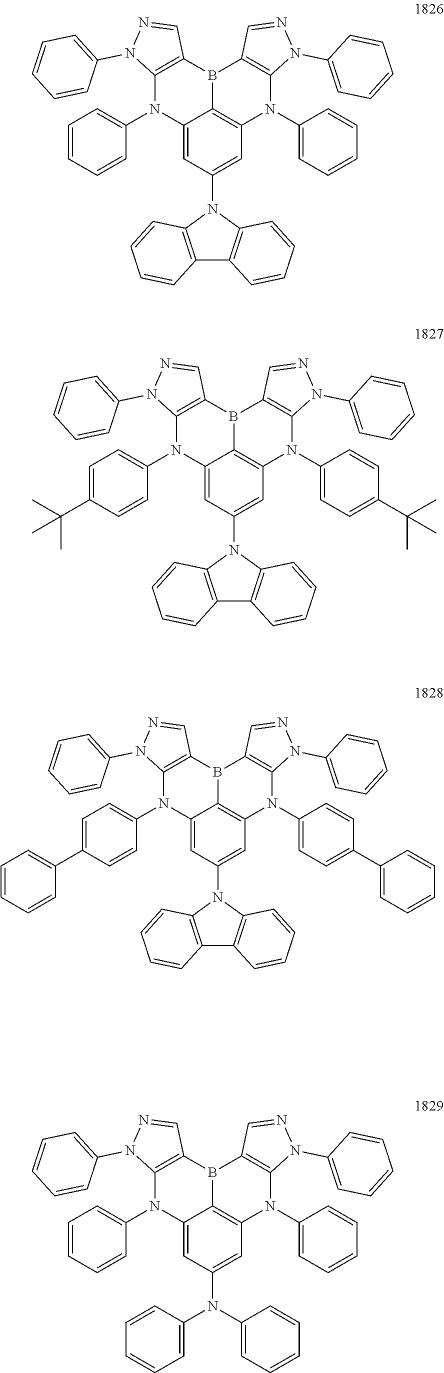 [SMILES: c1ccc(N2c3cc(-n4c5ccccc5c5ccccc54)cc4c3B(c3cnn(-c5ccccc5)c32)c2cnn(-c3ccccc3)c2N4c2ccccc2)cc1 ; CC(C)(C)c1ccc(N2c3cc(-n4c5ccccc5c5ccccc54)cc4c3B(c3cnn(-c5ccccc5)c32)c2cnn(-c3ccccc3)c2N4c2ccc(C(C)(C)C)cc2)cc1 ; c1ccc(-c2ccc(N3c4cc(-n5c6ccccc6c6ccccc65)cc5c4B(c4cnn(-c6ccccc6)c43)c3cnn(-c4ccccc4)c3N5c3ccc(-c4ccccc4)cc3)cc2)cc1 ; c1ccc(N(c2ccccc2)c2cc3c4c(c2)N(c2ccccc2)c2c(cnn2-c2ccccc2)B4c2cnn(-c4ccccc4)c2N3c2ccccc2)cc1]

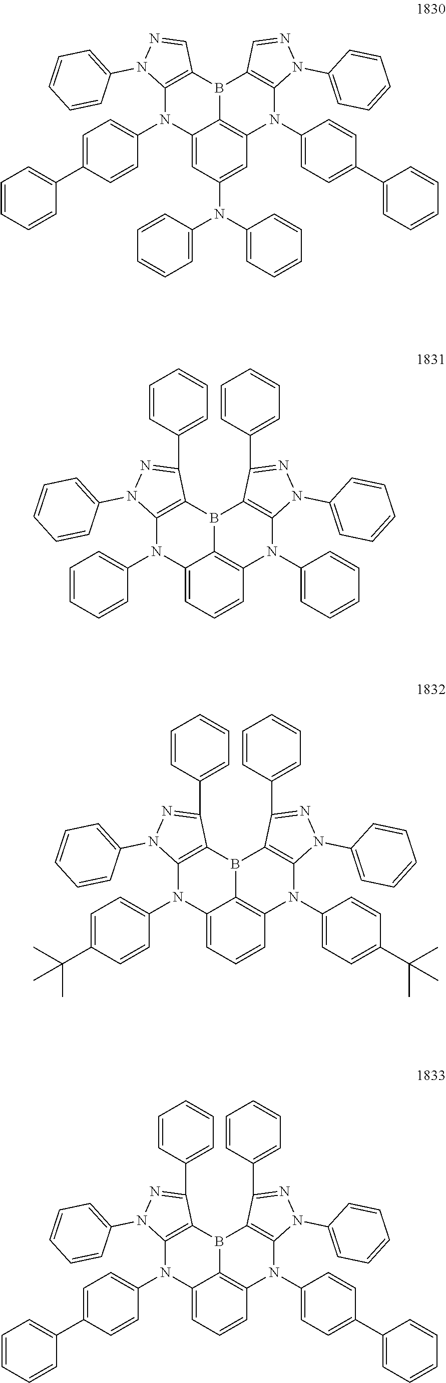 [SMILES: c1ccc(-c2ccc(N3c4cc(N(c5ccccc5)c5ccccc5)cc5c4B(c4cnn(-c6ccccc6)c43)c3cnn(-c4ccccc4)c3N5c3ccc(-c4ccccc4)cc3)cc2)cc1 ; c1ccc(-c2nn(-c3ccccc3)c3c2B2c4c(cccc4N(c4ccccc4)c4c2c(-c2ccccc2)nn4-c2ccccc2)N3c2ccccc2)cc1 ; CC(C)(C)c1ccc(N2c3cccc4c3B(c3c(-c5ccccc5)nn(-c5ccccc5)c32)c2c(-c3ccccc3)nn(-c3ccccc3)c2N4c2ccc(C(C)(C)C)cc2)cc1 ; c1ccc(-c2ccc(N3c4cccc5c4B(c4c(-c6ccccc6)nn(-c6ccccc6)c43)c3c(-c4ccccc4)nn(-c4ccccc4)c3N5c3ccc(-c4ccccc4)cc3)cc2)cc1]

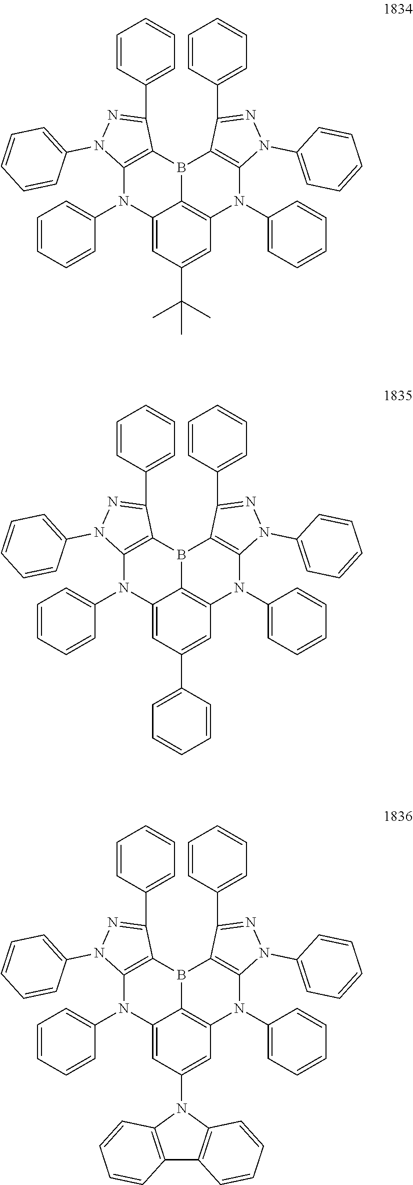 [SMILES: CC(C)(C)c1cc2c3c(c1)N(c1ccccc1)c1c(c(-c4ccccc4)nn1-c1ccccc1)B3c1c(-c3ccccc3)nn(-c3ccccc3)c1N2c1ccccc1 ; c1ccc(-c2cc3c4c(c2)N(c2ccccc2)c2c(c(-c5ccccc5)nn2-c2ccccc2)B4c2c(-c4ccccc4)nn(-c4ccccc4)c2N3c2ccccc2)cc1 ; c1ccc(-c2nn(-c3ccccc3)c3c2B2c4c(cc(-n5c6ccccc6c6ccccc65)cc4N(c4ccccc4)c4c2c(-c2ccccc2)nn4-c2ccccc2)N3c2ccccc2)cc1]

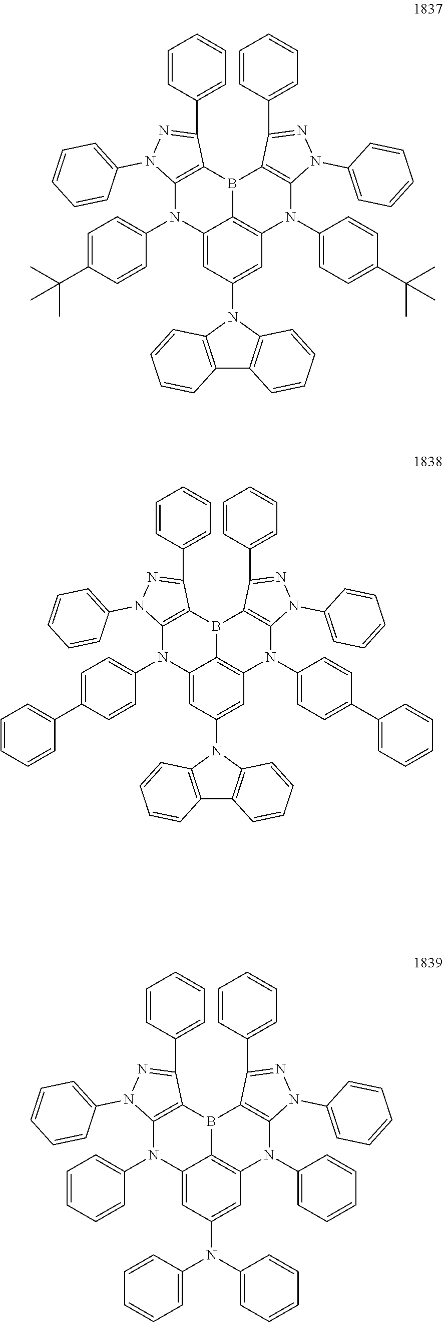 [SMILES: CC(C)(C)c1ccc(N2c3cc(-n4c5ccccc5c5ccccc54)cc4c3B(c3c(-c5ccccc5)nn(-c5ccccc5)c32)c2c(-c3ccccc3)nn(-c3ccccc3)c2N4c2ccc(C(C)(C)C)cc2)cc1 ; c1ccc(-c2ccc(N3c4cc(-n5c6ccccc6c6ccccc65)cc5c4B(c4c(-c6ccccc6)nn(-c6ccccc6)c43)c3c(-c4ccccc4)nn(-c4ccccc4)c3N5c3ccc(-c4ccccc4)cc3)cc2)cc1 ; c1ccc(-c2nn(-c3ccccc3)c3c2B2c4c(cc(N(c5ccccc5)c5ccccc5)cc4N(c4ccccc4)c4c2c(-c2ccccc2)nn4-c2ccccc2)N3c2ccccc2)cc1]

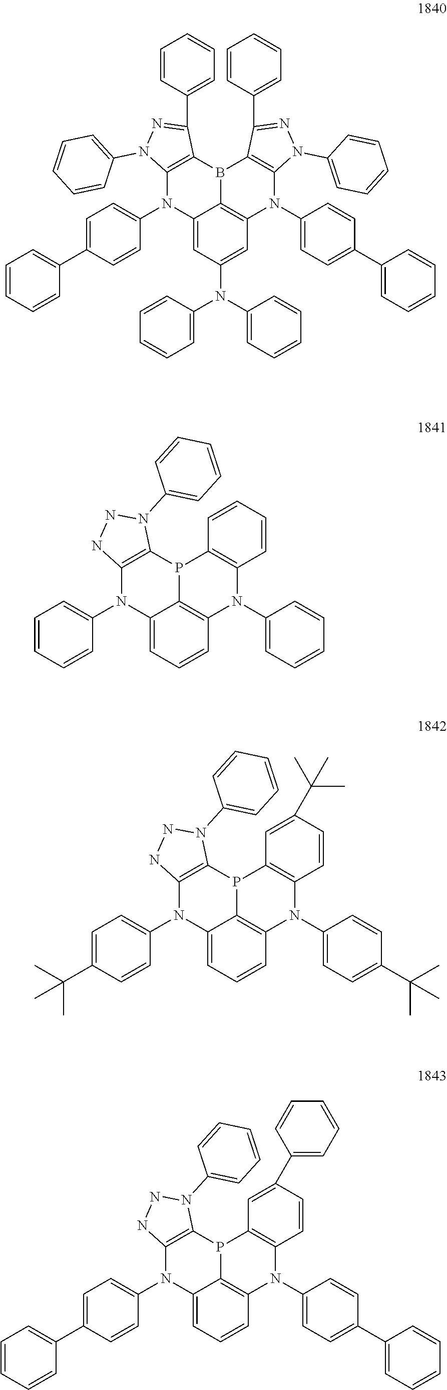 [SMILES: c1ccc(-c2ccc(N3c4cc(N(c5ccccc5)c5ccccc5)cc5c4B(c4c(-c6ccccc6)nn(-c6ccccc6)c43)c3c(-c4ccccc4)nn(-c4ccccc4)c3N5c3ccc(-c4ccccc4)cc3)cc2)cc1 ; c1ccc(N2c3ccccc3-p3c4n(-c5ccccc5)[nH][nH]c=4n(-c4ccccc4)c4cccc2c43)cc1 ; CC(C)(C)c1ccc(N2c3ccc(C(C)(C)C)cc3-p3c4n(-c5ccccc5)[nH][nH]c=4n(-c4ccc(C(C)(C)C)cc4)c4cccc2c43)cc1 ; c1ccc(-c2ccc(N3c4ccc(-c5ccccc5)cc4-p4c5n(-c6ccccc6)[nH][nH]c=5n(-c5ccc(-c6ccccc6)cc5)c5cccc3c54)cc2)cc1]

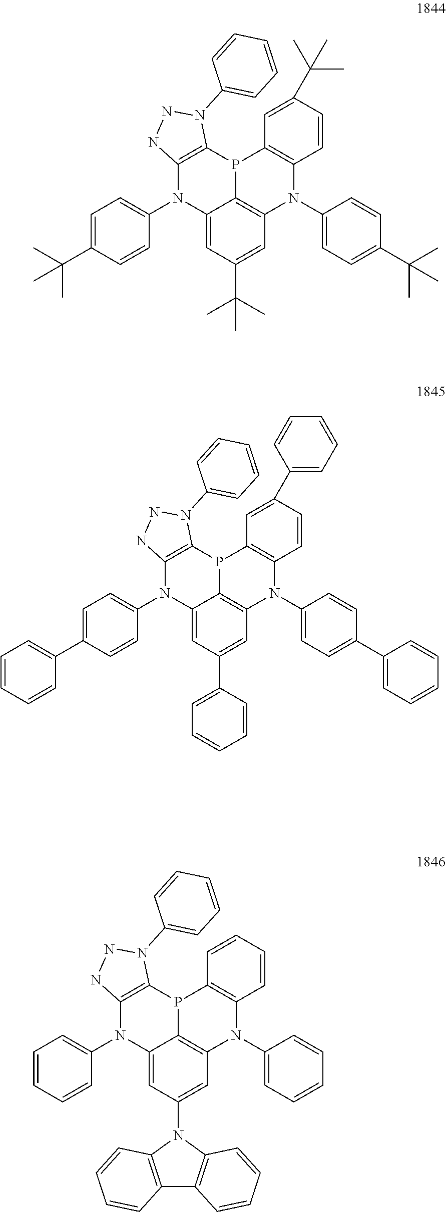 [SMILES: CC(C)(C)c1ccc(N2c3ccc(C(C)(C)C)cc3-p3c4n(-c5ccccc5)[nH][nH]c=4n(-c4ccc(C(C)(C)C)cc4)c4cc(C(C)(C)C)cc2c43)cc1 ; c1ccc(-c2ccc(N3c4ccc(-c5ccccc5)cc4-p4c5n(-c6ccccc6)[nH][nH]c=5n(-c5ccc(-c6ccccc6)cc5)c5cc(-c6ccccc6)cc3c54)cc2)cc1 ; c1ccc(N2c3ccccc3-p3c4n(-c5ccccc5)[nH][nH]c=4n(-c4ccccc4)c4cc(-n5c6ccccc6c6ccccc65)cc2c43)cc1]

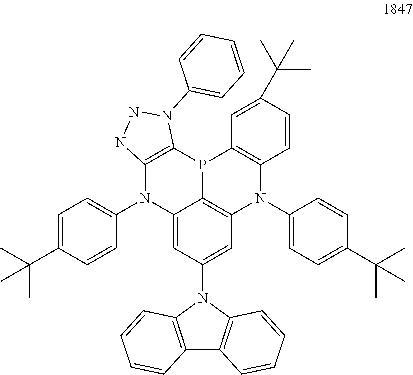 [SMILES: CC(C)(C)c1ccc(N2c3ccc(C(C)(C)C)cc3-p3c4n(-c5ccccc5)[nH][nH]c=4n(-c4ccc(C(C)(C)C)cc4)c4cc(-n5c6ccccc6c6ccccc65)cc2c43)cc1]

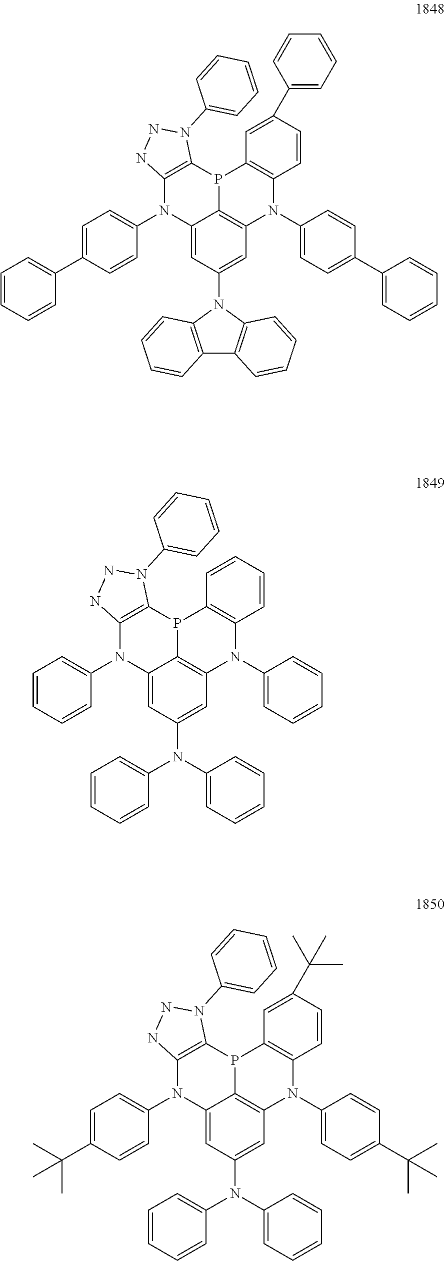 [SMILES: c1ccc(-c2ccc(N3c4ccc(-c5ccccc5)cc4-p4c5n(-c6ccccc6)[nH][nH]c=5n(-c5ccc(-c6ccccc6)cc5)c5cc(-n6c7ccccc7c7ccccc76)cc3c54)cc2)cc1 ; c1ccc(N(c2ccccc2)c2cc3c4c(c2)n(-c2ccccc2)c2[nH][nH]n(-c5ccccc5)c=2p4-c2ccccc2N3c2ccccc2)cc1 ; CC(C)(C)c1ccc(N2c3ccc(C(C)(C)C)cc3-p3c4n(-c5ccccc5)[nH][nH]c=4n(-c4ccc(C(C)(C)C)cc4)c4cc(N(c5ccccc5)c5ccccc5)cc2c43)cc1]

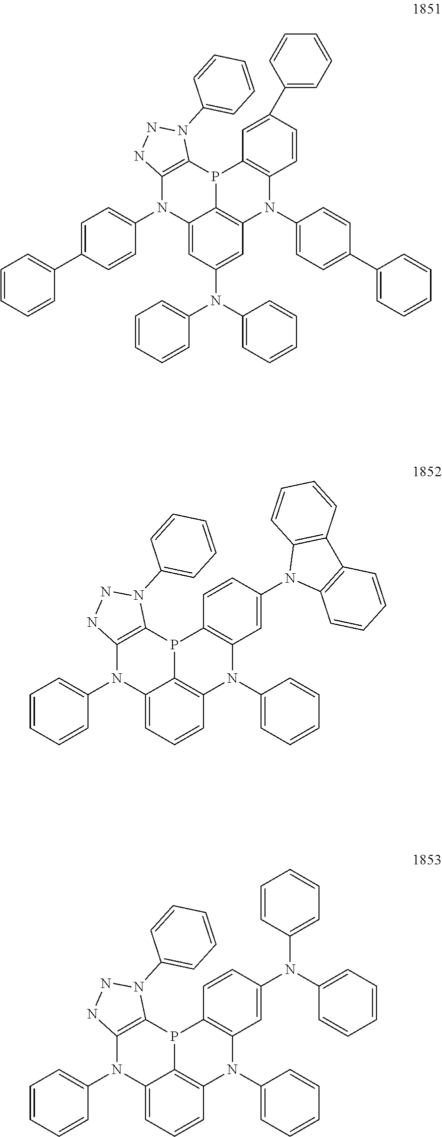 [SMILES: c1ccc(-c2ccc(N3c4ccc(-c5ccccc5)cc4-p4c5n(-c6ccccc6)[nH][nH]c=5n(-c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3-p3c4n(-c5ccccc5)[nH][nH]c=4n(-c4ccccc4)c4cccc2c43)cc1 ; c1ccc(N(c2ccccc2)c2ccc3c(c2)N(c2ccccc2)c2cccc4c2p-3c2n(-c3ccccc3)[nH][nH]c=2n4-c2ccccc2)cc1]

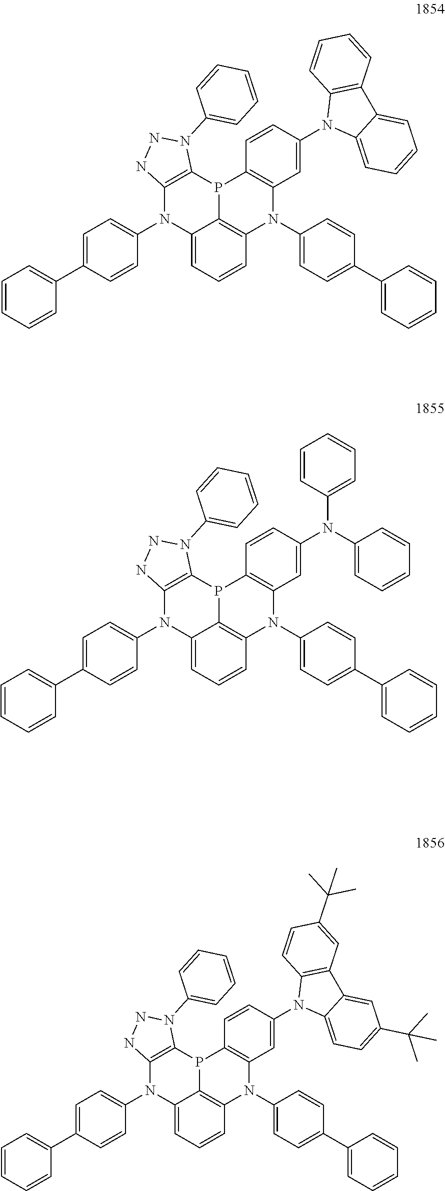 [SMILES: c1ccc(-c2ccc(N3c4cc(-n5c6ccccc6c6ccccc65)ccc4-p4c5n(-c6ccccc6)[nH][nH]c=5n(-c5ccc(-c6ccccc6)cc5)c5cccc3c54)cc2)cc1 ; c1ccc(-c2ccc(N3c4cc(N(c5ccccc5)c5ccccc5)ccc4-p4c5n(-c6ccccc6)[nH][nH]c=5n(-c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1p-2c1n(-c2ccccc2)[nH][nH]c=1n3-c1ccc(-c2ccccc2)cc1]

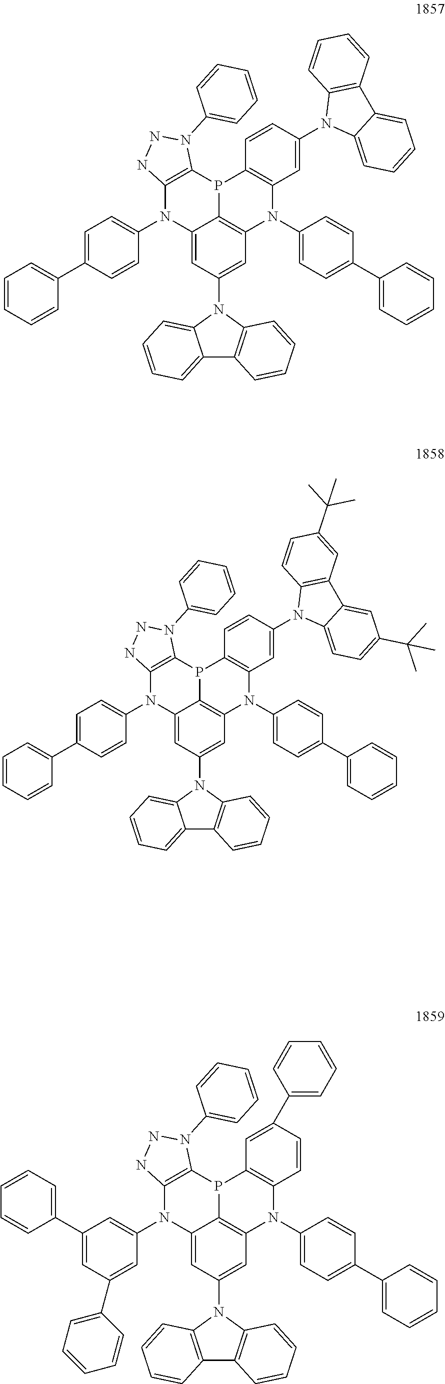 [SMILES: c1ccc(-c2ccc(N3c4cc(-n5c6ccccc6c6ccccc65)ccc4-p4c5n(-c6ccccc6)[nH][nH]c=5n(-c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1p-2c1n(-c2ccccc2)[nH][nH]c=1n3-c1ccc(-c2ccccc2)cc1 ; c1ccc(-c2ccc(N3c4ccc(-c5ccccc5)cc4-p4c5n(-c6ccccc6)[nH][nH]c=5n(-c5cc(-c6ccccc6)cc(-c6ccccc6)c5)c5cc(-n6c7ccccc7c7ccccc76)cc3c54)cc2)cc1]

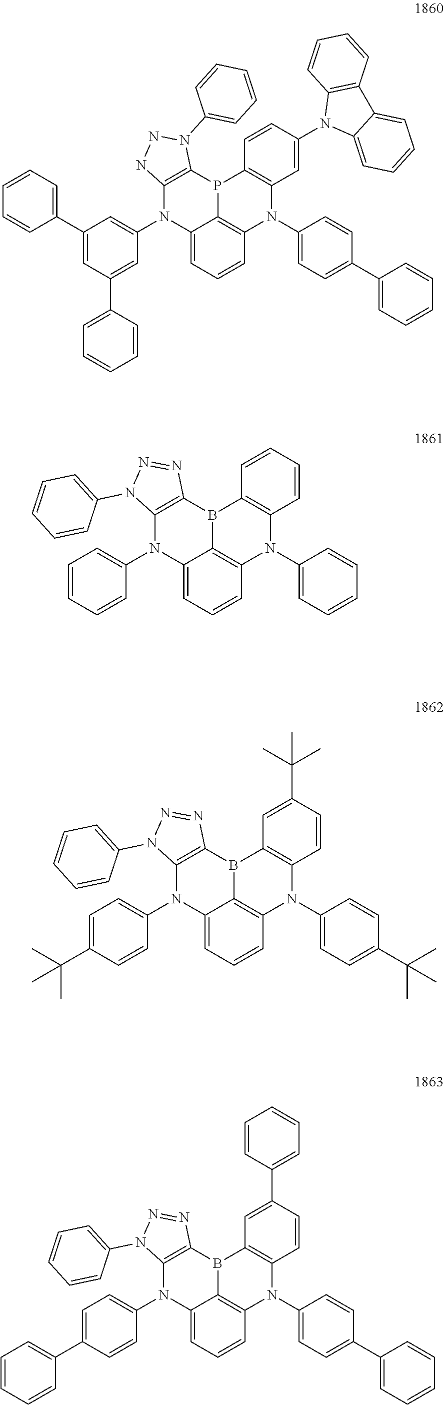 [SMILES: c1ccc(-c2ccc(N3c4cc(-n5c6ccccc6c6ccccc65)ccc4-p4c5n(-c6ccccc6)[nH][nH]c=5n(-c5cc(-c6ccccc6)cc(-c6ccccc6)c5)c5cccc3c54)cc2)cc1 ; c1ccc(N2c3ccccc3B3c4nnn(-c5ccccc5)c4N(c4ccccc4)c4cccc2c43)cc1 ; CC(C)(C)c1ccc(N2c3ccc(C(C)(C)C)cc3B3c4nnn(-c5ccccc5)c4N(c4ccc(C(C)(C)C)cc4)c4cccc2c43)cc1 ; c1ccc(-c2ccc(N3c4ccc(-c5ccccc5)cc4B4c5nnn(-c6ccccc6)c5N(c5ccc(-c6ccccc6)cc5)c5cccc3c54)cc2)cc1]

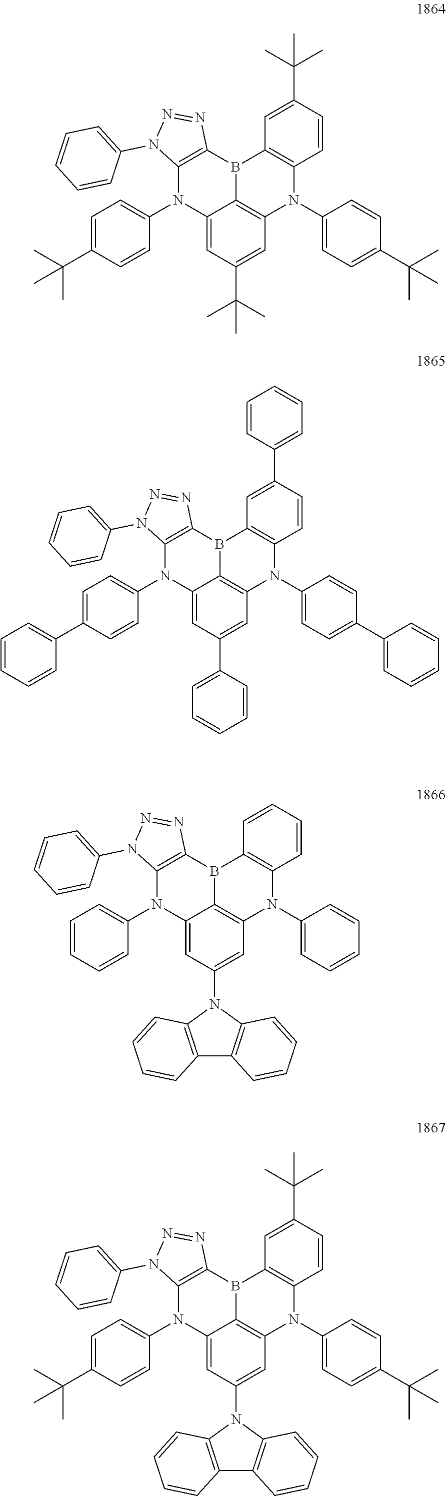

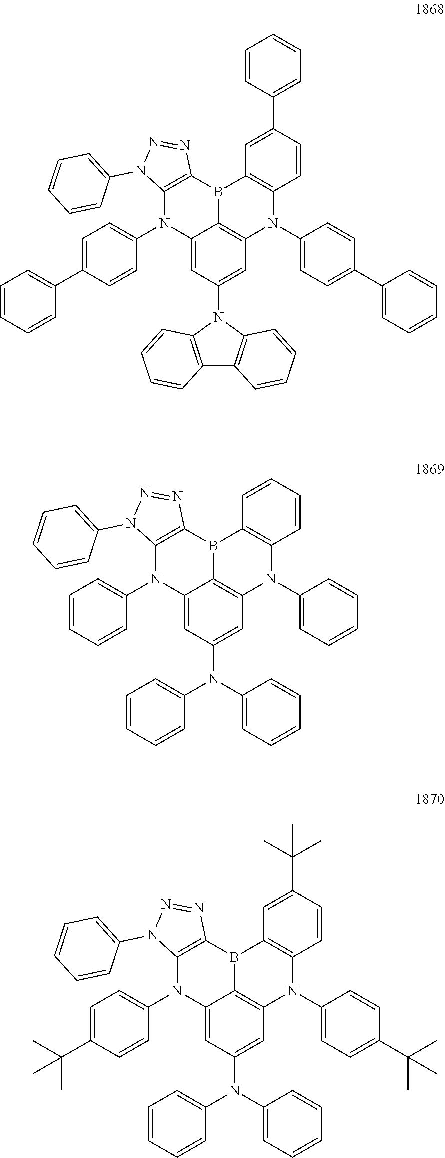 [SMILES: c1ccc(-c2ccc(N3c4ccc(-c5ccccc5)cc4B4c5nnn(-c6ccccc6)c5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2c(nnn2-c2ccccc2)B4c2ccccc2N3c2ccccc2)cc1 ; CC(C)(C)c1ccc(N2c3ccc(C(C)(C)C)cc3B3c4nnn(-c5ccccc5)c4N(c4ccc(C(C)(C)C)cc4)c4cc(N(c5ccccc5)c5ccccc5)cc2c43)cc1]

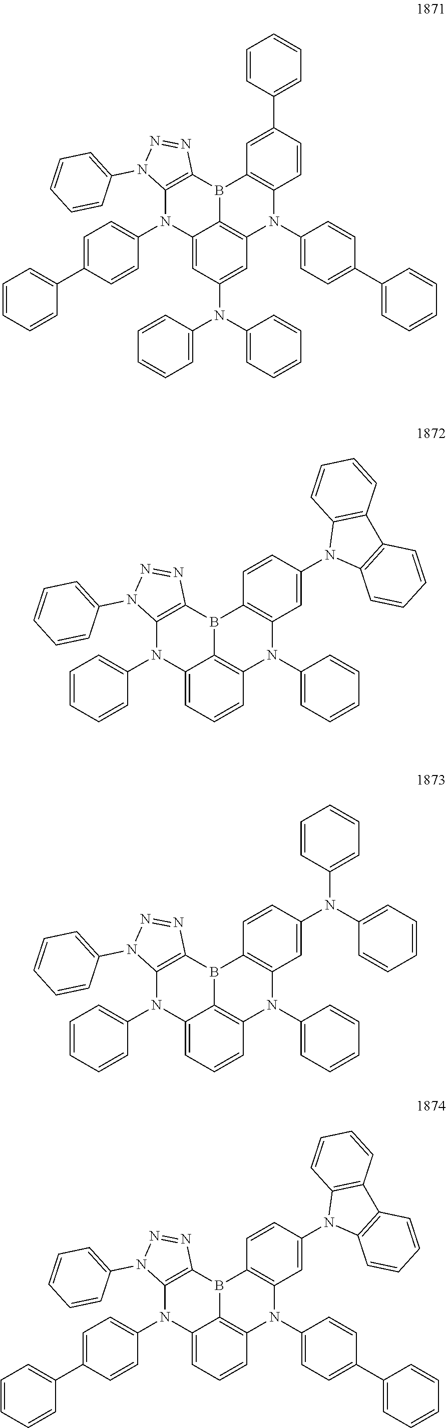 [SMILES: c1ccc(-c2ccc(N3c4ccc(-c5ccccc5)cc4B4c5nnn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nnn(-c5ccccc5)c4N(c4ccccc4)c4cccc2c43)cc1 ; c1ccc(N(c2ccccc2)c2ccc3c(c2)N(c2ccccc2)c2cccc4c2B3c2nnn(-c3ccccc3)c2N4c2ccccc2)cc1 ; c1ccc(-c2ccc(N3c4cc(-n5c6ccccc6c6ccccc65)ccc4B4c5nnn(-c6ccccc6)c5N(c5ccc(-c6ccccc6)cc5)c5cccc3c54)cc2)cc1]

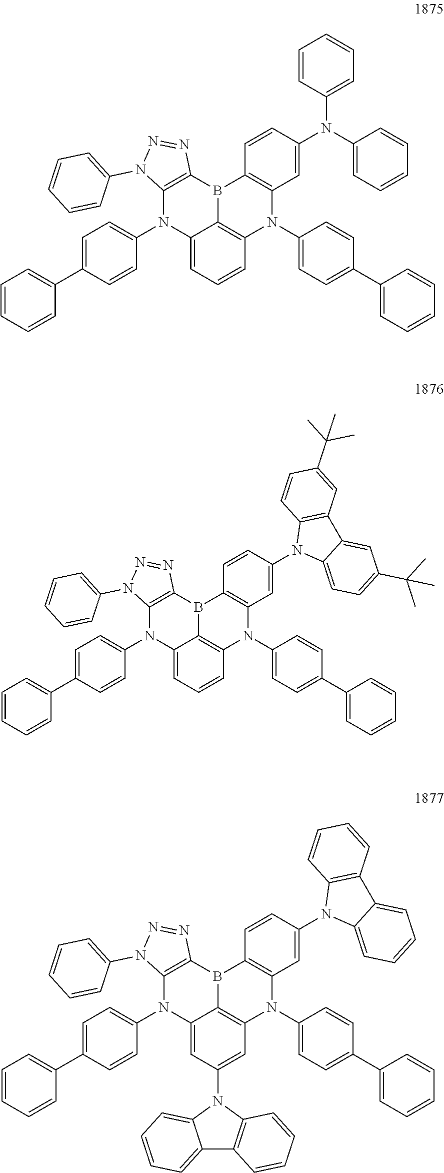 [SMILES: c1ccc(-c2ccc(N3c4cc(N(c5ccccc5)c5ccccc5)ccc4B4c5nnn(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nnn(-c2ccccc2)c1N3c1ccc(-c2ccccc2)cc1 ; c1ccc(-c2ccc(N3c4cc(-n5c6ccccc6c6ccccc65)ccc4B4c5nnn(-c6ccccc6)c5N(c5ccc(-c6ccccc6)cc5)c5cc(-n6c7ccccc7c7ccccc76)cc3c54)cc2)cc1]

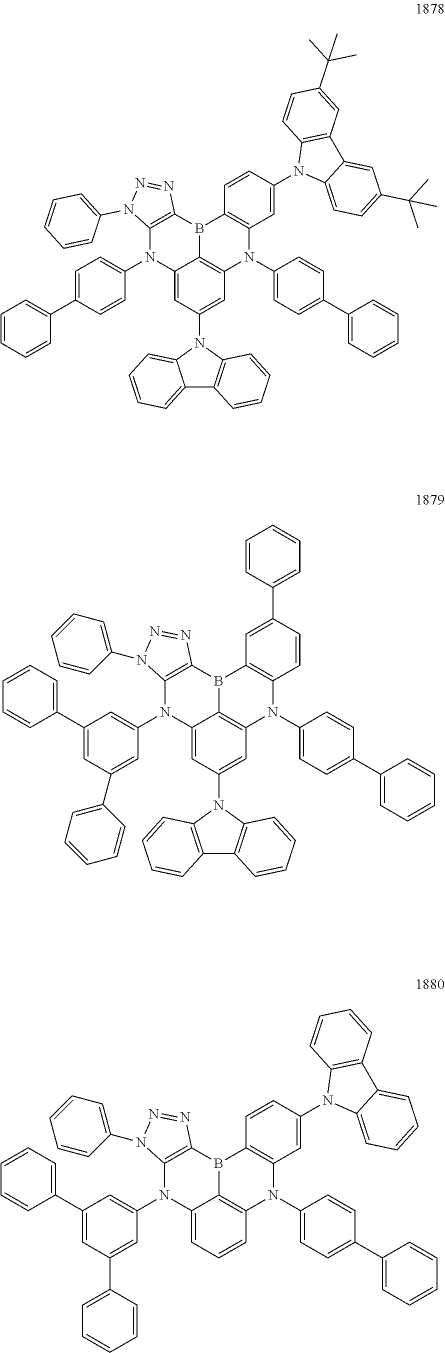 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nnn(-c2ccccc2)c1N3c1ccc(-c2ccccc2)cc1 ; c1ccc(-c2ccc(N3c4ccc(-c5ccccc5)cc4B4c5nnn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nnn(-c6ccccc6)c5N(c5cc(-c6ccccc6)cc(-c6ccccc6)c5)c5cccc3c54)cc2)cc1]

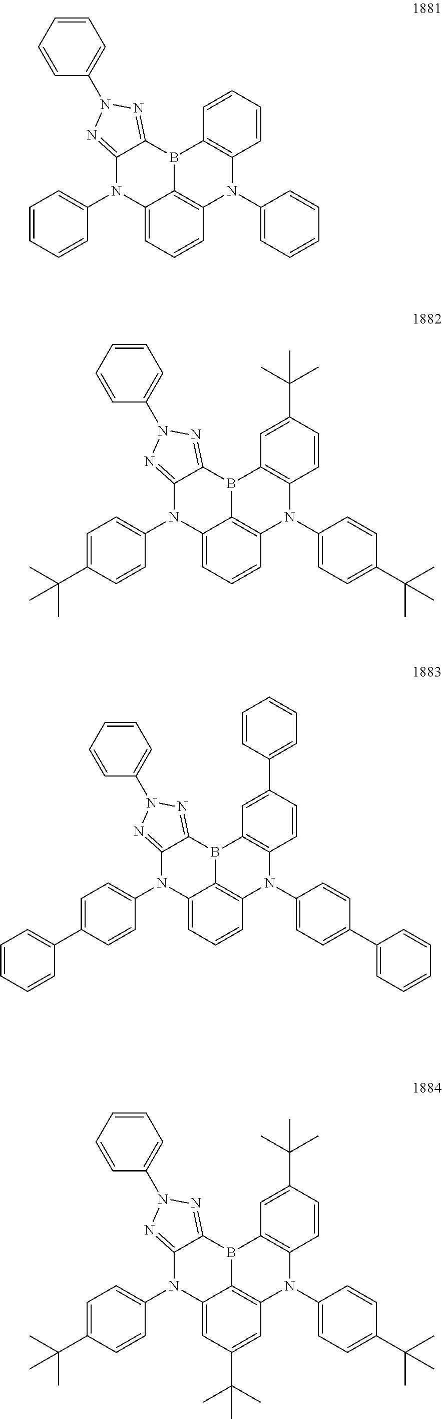

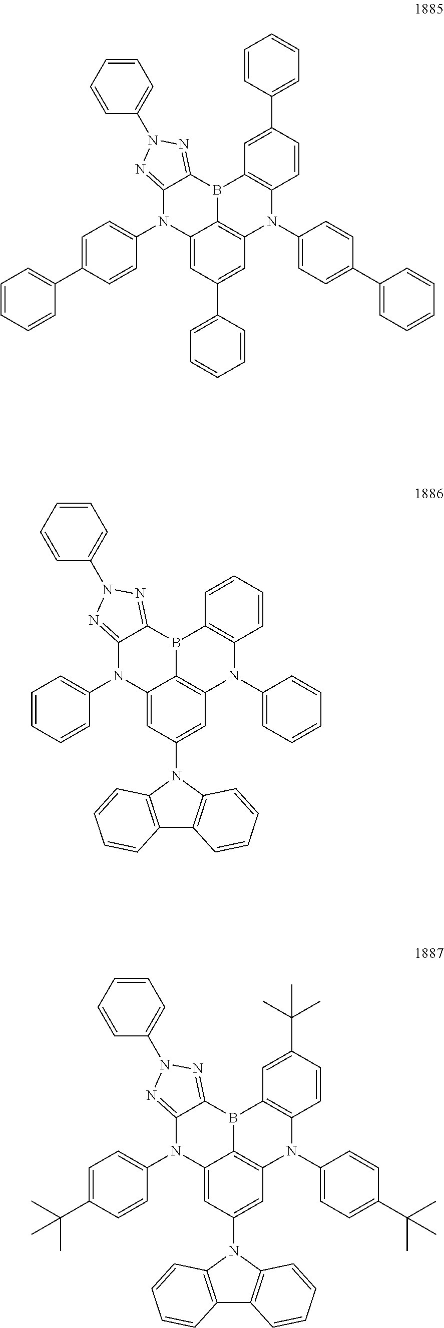 [SMILES: c1ccc(-c2ccc(N3c4ccc(-c5ccccc5)cc4B4c5nn(-c6ccccc6)nc5N(c5ccc(-c6ccccc6)cc5)c5cc(-c6ccccc6)cc3c54)cc2)cc1 ; c1ccc(N2c3ccccc3B3c4nn(-c5ccccc5)nc4N(c4ccccc4)c4cc(-n5c6ccccc6c6ccccc65)cc2c43)cc1 ; CC(C)(C)c1ccc(N2c3ccc(C(C)(C)C)cc3B3c4nn(-c5ccccc5)nc4N(c4ccc(C(C)(C)C)cc4)c4cc(-n5c6ccccc6c6ccccc65)cc2c43)cc1]

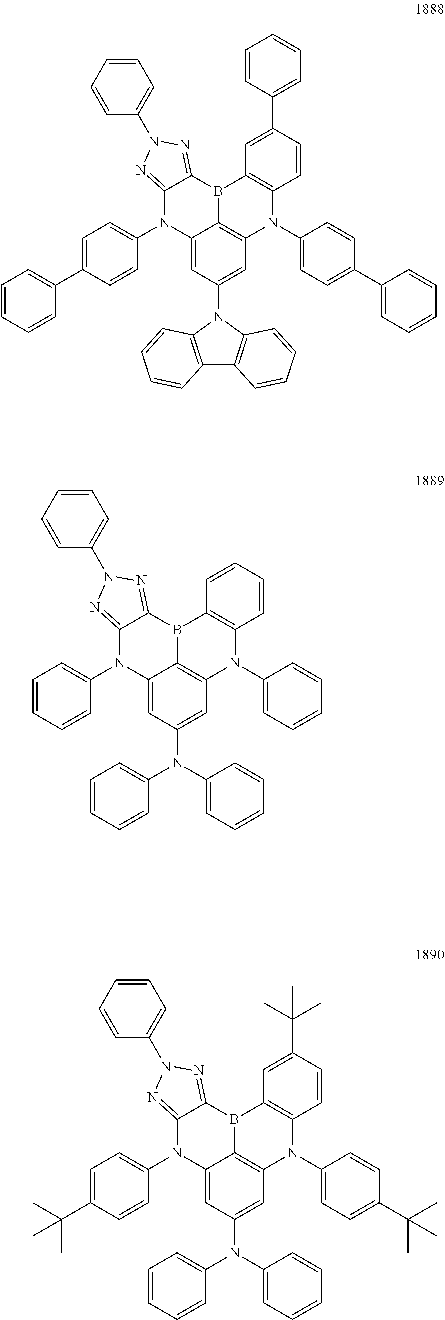 [SMILES: c1ccc(-c2ccc(N3c4ccc(-c5ccccc5)cc4B4c5nn(-c6ccccc6)nc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2nn(-c5ccccc5)nc2B4c2ccccc2N3c2ccccc2)cc1 ; CC(C)(C)c1ccc(N2c3ccc(C(C)(C)C)cc3B3c4nn(-c5ccccc5)nc4N(c4ccc(C(C)(C)C)cc4)c4cc(N(c5ccccc5)c5ccccc5)cc2c43)cc1]

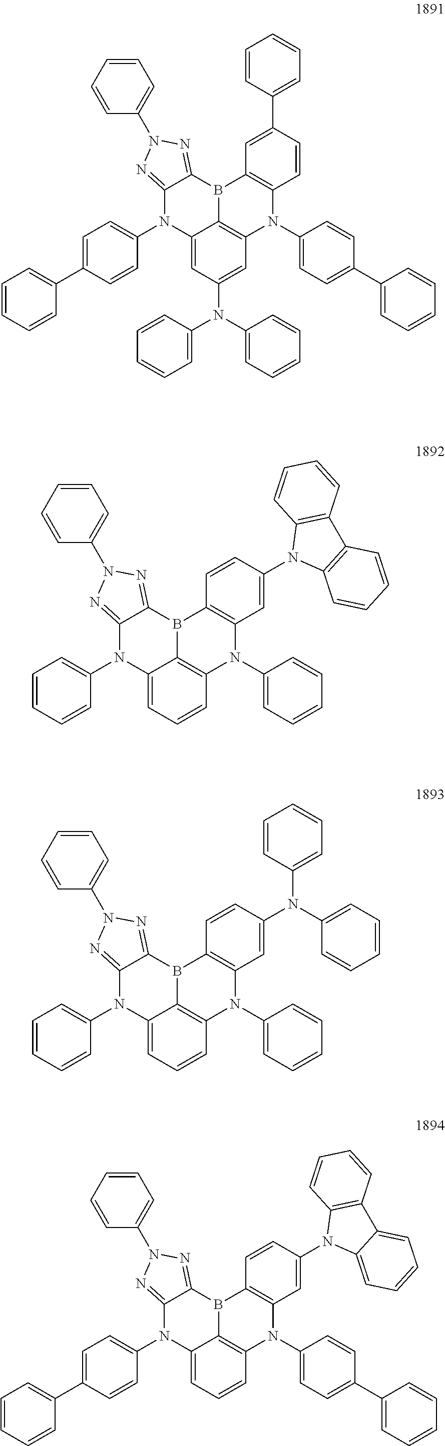 [SMILES: c1ccc(-c2ccc(N3c4ccc(-c5ccccc5)cc4B4c5nn(-c6ccccc6)nc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nn(-c5ccccc5)nc4N(c4ccccc4)c4cccc2c43)cc1 ; c1ccc(N(c2ccccc2)c2ccc3c(c2)N(c2ccccc2)c2cccc4c2B3c2nn(-c3ccccc3)nc2N4c2ccccc2)cc1 ; c1ccc(-c2ccc(N3c4cc(-n5c6ccccc6c6ccccc65)ccc4B4c5nn(-c6ccccc6)nc5N(c5ccc(-c6ccccc6)cc5)c5cccc3c54)cc2)cc1]

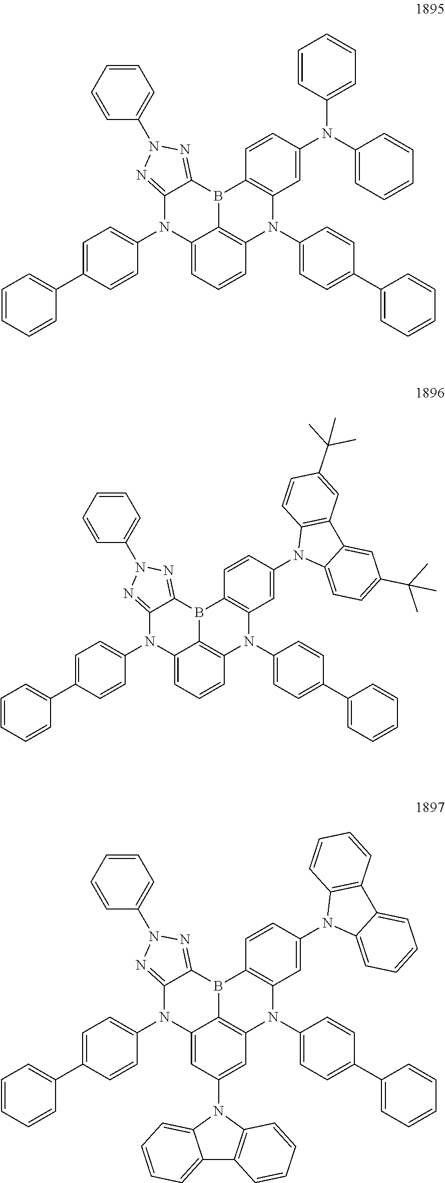 [SMILES: c1ccc(-c2ccc(N3c4cc(N(c5ccccc5)c5ccccc5)ccc4B4c5nn(-c6ccccc6)nc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nn(-c2ccccc2)nc1N3c1ccc(-c2ccccc2)cc1 ; c1ccc(-c2ccc(N3c4cc(-n5c6ccccc6c6ccccc65)ccc4B4c5nn(-c6ccccc6)nc5N(c5ccc(-c6ccccc6)cc5)c5cc(-n6c7ccccc7c7ccccc76)cc3c54)cc2)cc1]

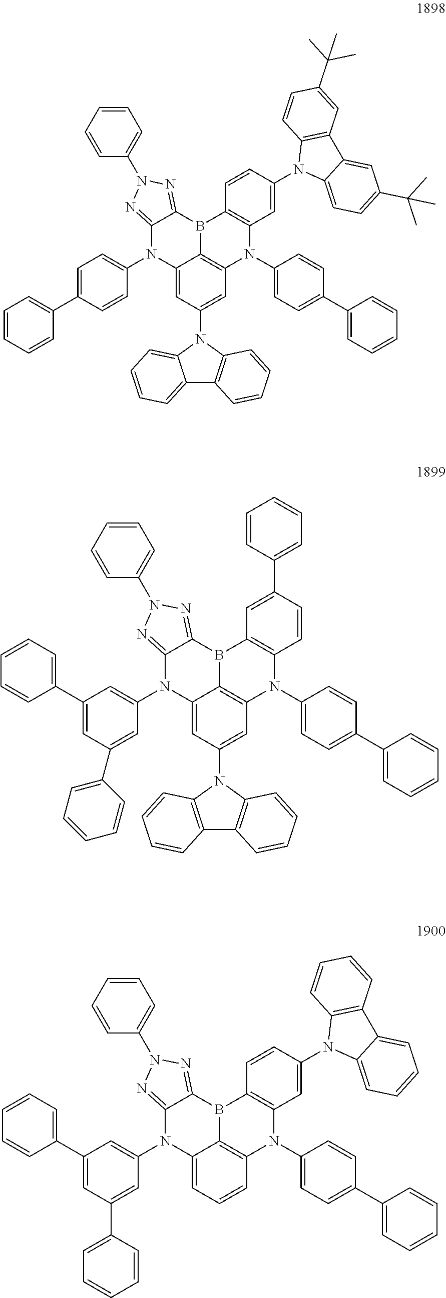 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nn(-c2ccccc2)nc1N3c1ccc(-c2ccccc2)cc1 ; c1ccc(-c2ccc(N3c4ccc(-c5ccccc5)cc4B4c5nn(-c6ccccc6)nc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nn(-c6ccccc6)nc5N(c5cc(-c6ccccc6)cc(-c6ccccc6)c5)c5cccc3c54)cc2)cc1]

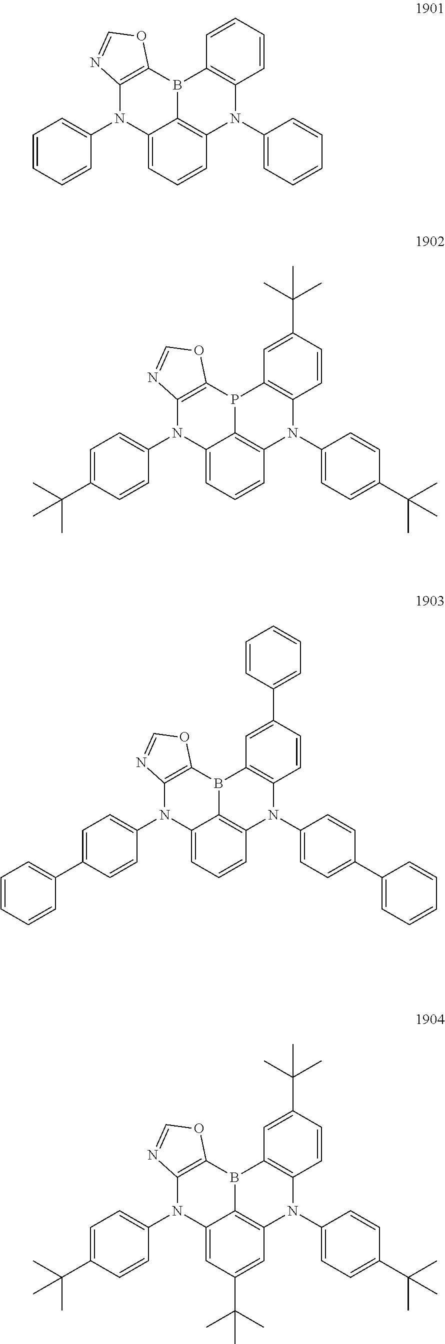

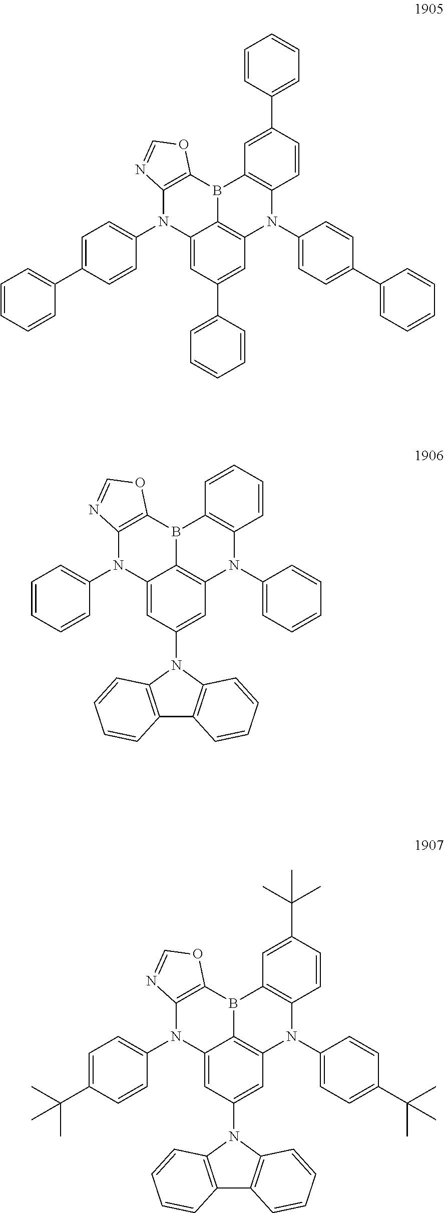 [SMILES: c1ccc(-c2ccc(N3c4ccc(-c5ccccc5)cc4B4c5ocnc5N(c5ccc(-c6ccccc6)cc5)c5cc(-c6ccccc6)cc3c54)cc2)cc1 ; c1ccc(N2c3ccccc3B3c4ocnc4N(c4ccccc4)c4cc(-n5c6ccccc6c6ccccc65)cc2c43)cc1 ; CC(C)(C)c1ccc(N2c3ccc(C(C)(C)C)cc3B3c4ocnc4N(c4ccc(C(C)(C)C)cc4)c4cc(-n5c6ccccc6c6ccccc65)cc2c43)cc1]

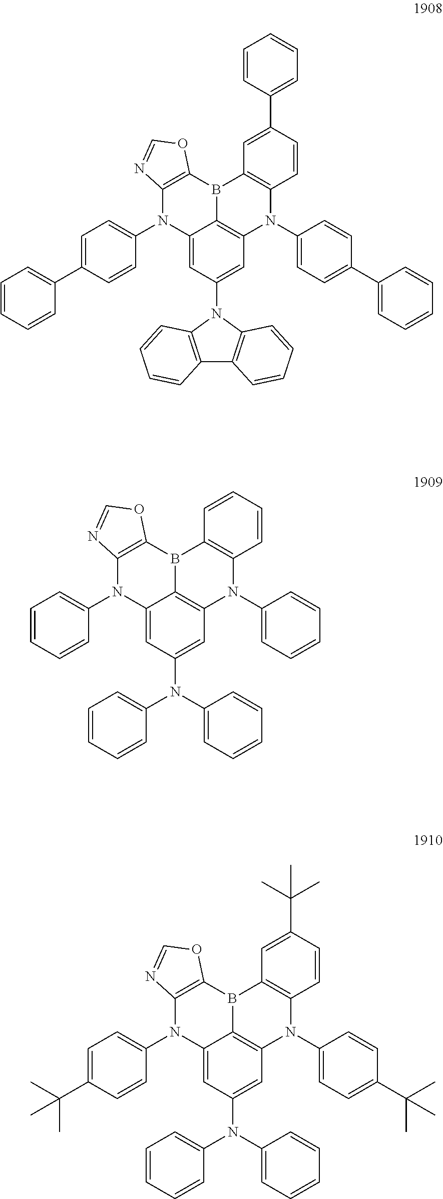 [SMILES: c1ccc(-c2ccc(N3c4ccc(-c5ccccc5)cc4B4c5ocnc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2ncoc2B4c2ccccc2N3c2ccccc2)cc1 ; CC(C)(C)c1ccc(N2c3ccc(C(C)(C)C)cc3B3c4ocnc4N(c4ccc(C(C)(C)C)cc4)c4cc(N(c5ccccc5)c5ccccc5)cc2c43)cc1]

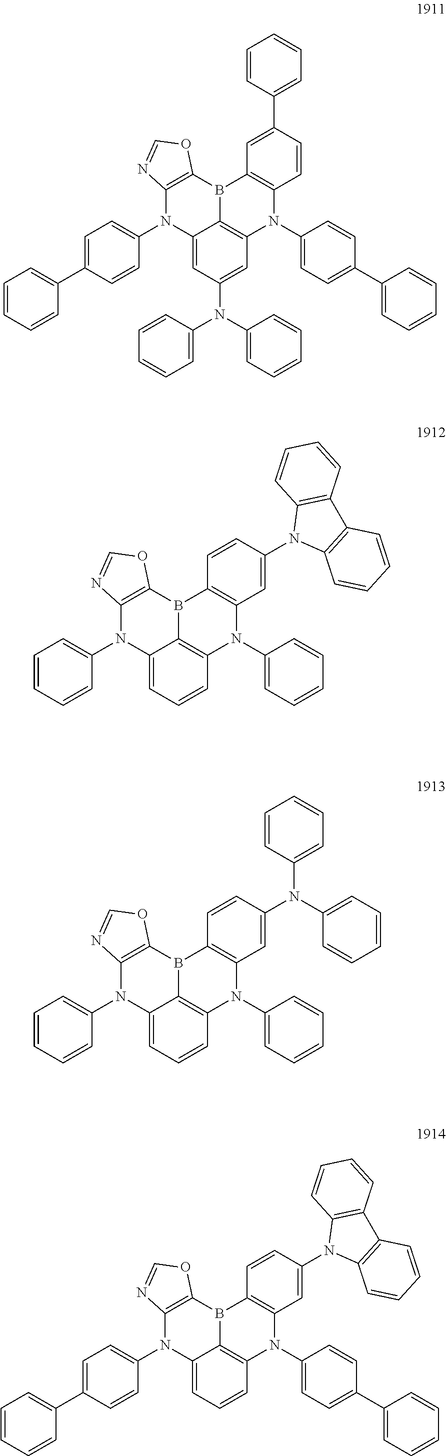 [SMILES: c1ccc(-c2ccc(N3c4ccc(-c5ccccc5)cc4B4c5ocnc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4ocnc4N(c4ccccc4)c4cccc2c43)cc1 ; c1ccc(N(c2ccccc2)c2ccc3c(c2)N(c2ccccc2)c2cccc4c2B3c2ocnc2N4c2ccccc2)cc1 ; c1ccc(-c2ccc(N3c4cc(-n5c6ccccc6c6ccccc65)ccc4B4c5ocnc5N(c5ccc(-c6ccccc6)cc5)c5cccc3c54)cc2)cc1]

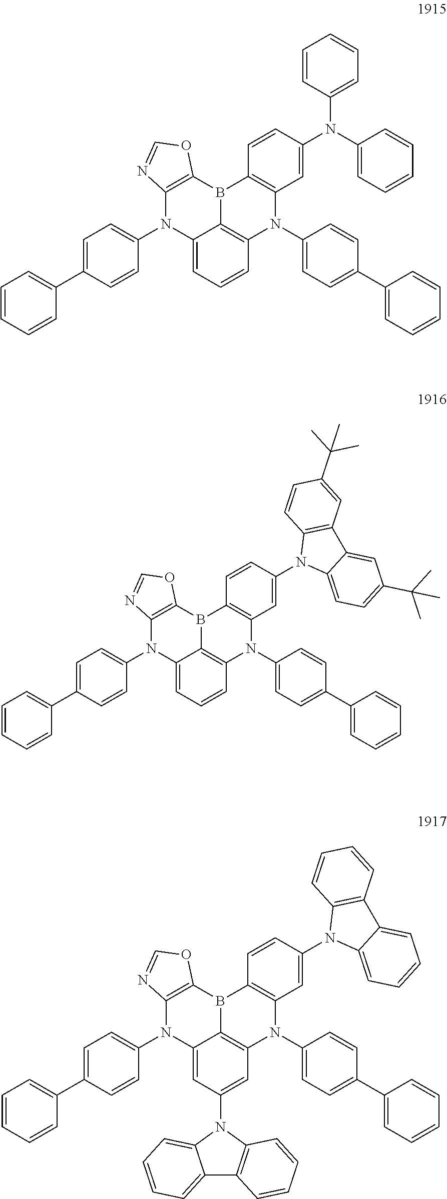 [SMILES: c1ccc(-c2ccc(N3c4cc(N(c5ccccc5)c5ccccc5)ccc4B4c5ocnc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ocnc1N3c1ccc(-c2ccccc2)cc1 ; c1ccc(-c2ccc(N3c4cc(-n5c6ccccc6c6ccccc65)ccc4B4c5ocnc5N(c5ccc(-c6ccccc6)cc5)c5cc(-n6c7ccccc7c7ccccc76)cc3c54)cc2)cc1]

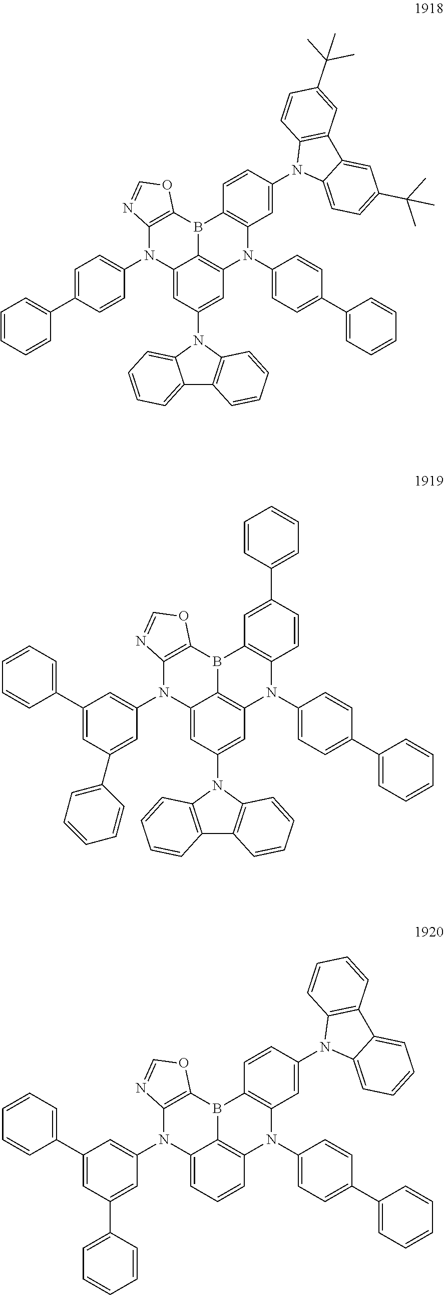 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ocnc1N3c1ccc(-c2ccccc2)cc1 ; c1ccc(-c2ccc(N3c4ccc(-c5ccccc5)cc4B4c5ocnc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ocnc5N(c5cc(-c6ccccc6)cc(-c6ccccc6)c5)c5cccc3c54)cc2)cc1]

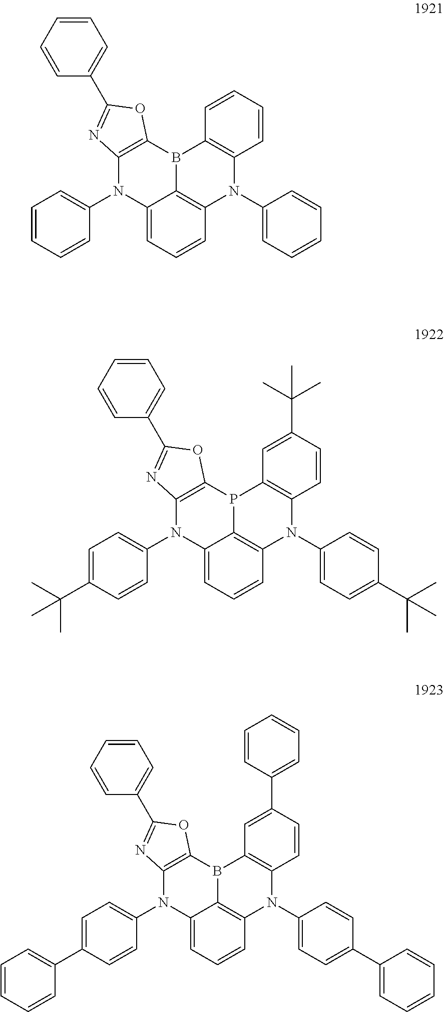 [SMILES: c1ccc(-c2nc3c(o2)B2c4ccccc4N(c4ccccc4)c4cccc(c42)N3c2ccccc2)cc1 ; CC(C)(C)c1ccc(N2c3ccc(C(C)(C)C)cc3P3c4oc(-c5ccccc5)nc4N(c4ccc(C(C)(C)C)cc4)c4cccc2c43)cc1 ; c1ccc(-c2ccc(N3c4ccc(-c5ccccc5)cc4B4c5oc(-c6ccccc6)nc5N(c5ccc(-c6ccccc6)cc5)c5cccc3c54)cc2)cc1]

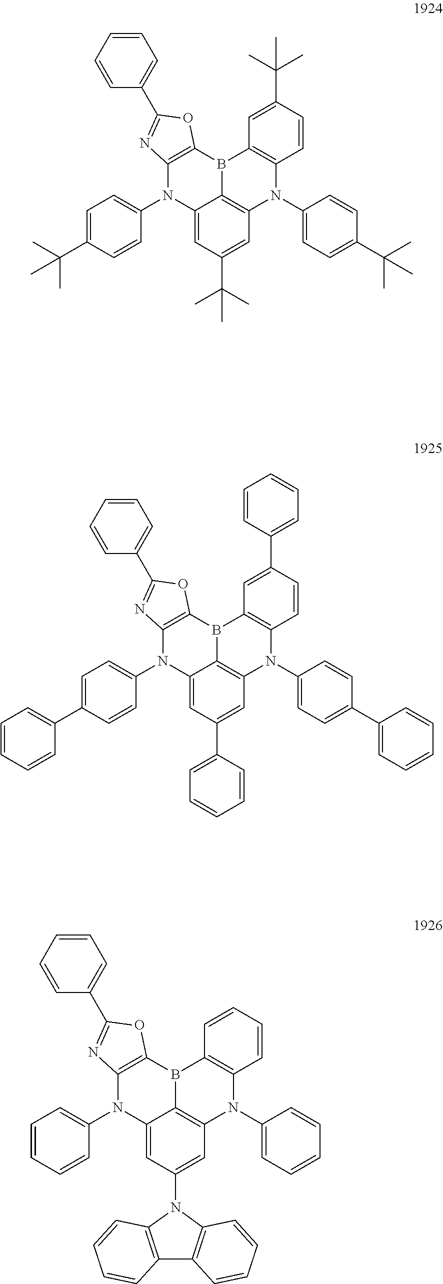 [SMILES: CC(C)(C)c1ccc(N2c3ccc(C(C)(C)C)cc3B3c4oc(-c5ccccc5)nc4N(c4ccc(C(C)(C)C)cc4)c4cc(C(C)(C)C)cc2c43)cc1 ; c1ccc(-c2ccc(N3c4ccc(-c5ccccc5)cc4B4c5oc(-c6ccccc6)nc5N(c5ccc(-c6ccccc6)cc5)c5cc(-c6ccccc6)cc3c54)cc2)cc1 ; c1ccc(-c2nc3c(o2)B2c4ccccc4N(c4ccccc4)c4cc(-n5c6ccccc6c6ccccc65)cc(c42)N3c2ccccc2)cc1]

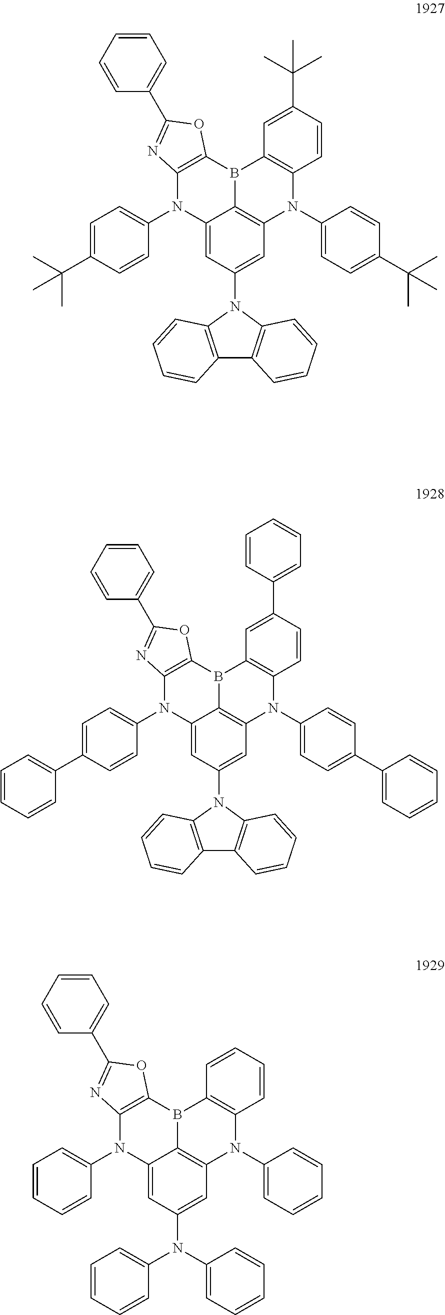 [SMILES: CC(C)(C)c1ccc(N2c3ccc(C(C)(C)C)cc3B3c4oc(-c5ccccc5)nc4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4B4c5oc(-c6ccccc6)nc5N(c5ccc(-c6ccccc6)cc5)c5cc(-n6c7ccccc7c7ccccc76)cc3c54)cc2)cc1 ; c1ccc(-c2nc3c(o2)B2c4ccccc4N(c4ccccc4)c4cc(N(c5ccccc5)c5ccccc5)cc(c42)N3c2ccccc2)cc1]

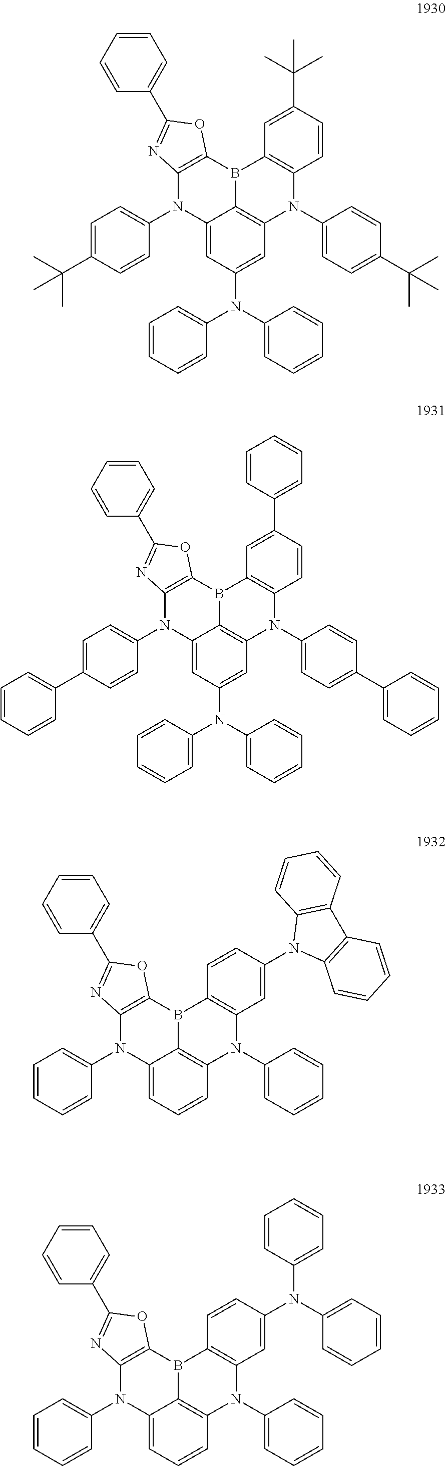 [SMILES: CC(C)(C)c1ccc(N2c3ccc(C(C)(C)C)cc3B3c4oc(-c5ccccc5)nc4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5oc(-c6ccccc6)nc5N(c5ccc(-c6ccccc6)cc5)c5cc(N(c6ccccc6)c6ccccc6)cc3c54)cc2)cc1 ; c1ccc(-c2nc3c(o2)B2c4ccc(-n5c6ccccc6c6ccccc65)cc4N(c4ccccc4)c4cccc(c42)N3c2ccccc2)cc1 ; c1ccc(-c2nc3c(o2)B2c4ccc(N(c5ccccc5)c5ccccc5)cc4N(c4ccccc4)c4cccc(c42)N3c2ccccc2)cc1]

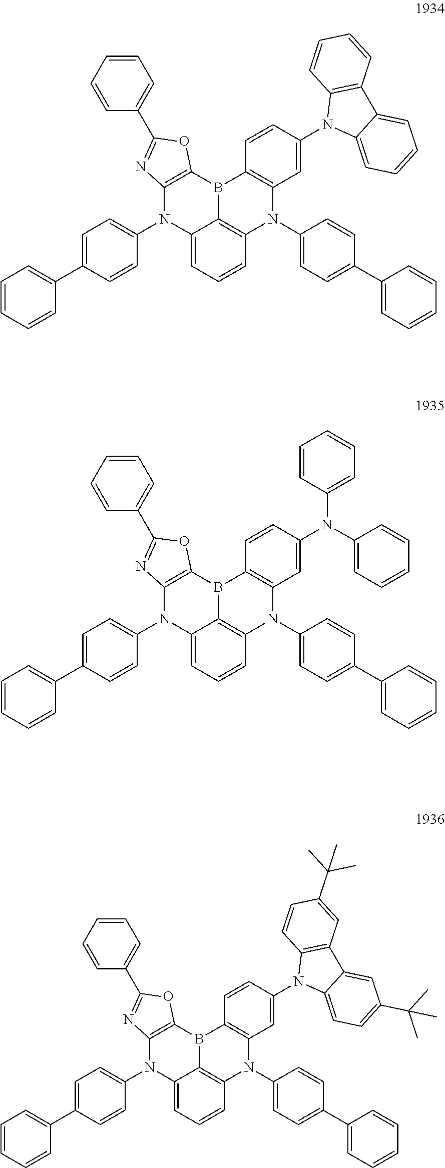 [SMILES: c1ccc(-c2ccc(N3c4cc(-n5c6ccccc6c6ccccc65)ccc4B4c5oc(-c6ccccc6)nc5N(c5ccc(-c6ccccc6)cc5)c5cccc3c54)cc2)cc1 ; c1ccc(-c2ccc(N3c4cc(N(c5ccccc5)c5ccccc5)ccc4B4c5oc(-c6ccccc6)nc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1oc(-c2ccccc2)nc1N3c1ccc(-c2ccccc2)cc1]

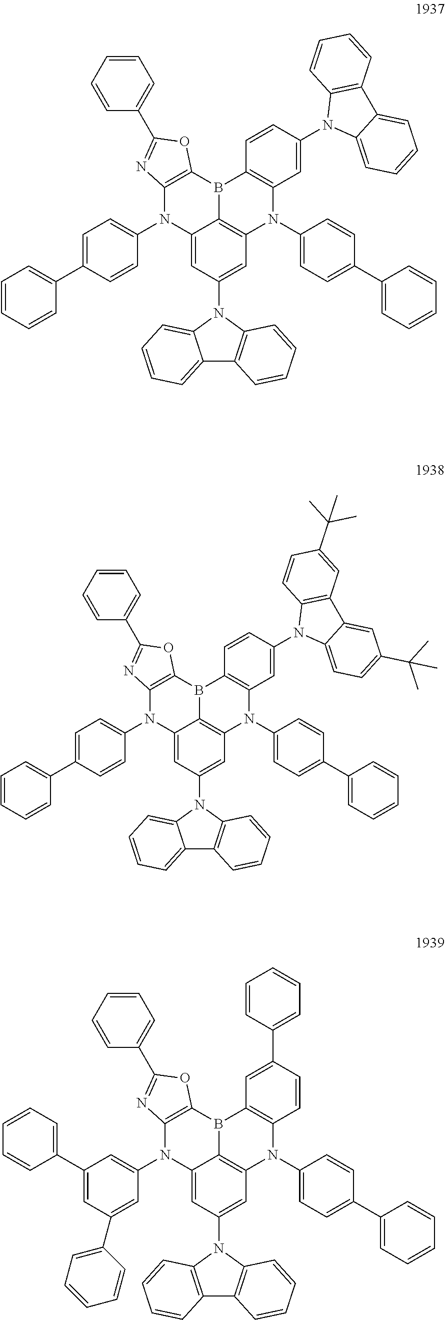 [SMILES: c1ccc(-c2ccc(N3c4cc(-n5c6ccccc6c6ccccc65)ccc4B4c5oc(-c6ccccc6)nc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1oc(-c2ccccc2)nc1N3c1ccc(-c2ccccc2)cc1 ; c1ccc(-c2ccc(N3c4ccc(-c5ccccc5)cc4B4c5oc(-c6ccccc6)nc5N(c5cc(-c6ccccc6)cc(-c6ccccc6)c5)c5cc(-n6c7ccccc7c7ccccc76)cc3c54)cc2)cc1]

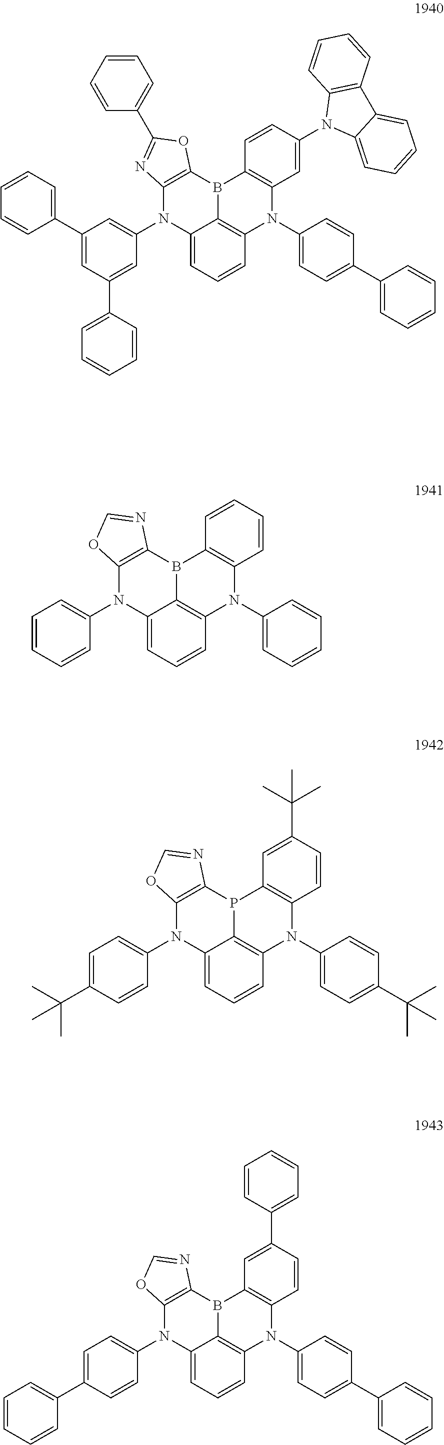 [SMILES: c1ccc(-c2ccc(N3c4cc(-n5c6ccccc6c6ccccc65)ccc4B4c5oc(-c6ccccc6)nc5N(c5cc(-c6ccccc6)cc(-c6ccccc6)c5)c5cccc3c54)cc2)cc1 ; c1ccc(N2c3ccccc3B3c4ncoc4N(c4ccccc4)c4cccc2c43)cc1 ; CC(C)(C)c1ccc(N2c3ccc(C(C)(C)C)cc3P3c4ncoc4N(c4ccc(C(C)(C)C)cc4)c4cccc2c43)cc1 ; c1ccc(-c2ccc(N3c4ccc(-c5ccccc5)cc4B4c5ncoc5N(c5ccc(-c6ccccc6)cc5)c5cccc3c54)cc2)cc1]

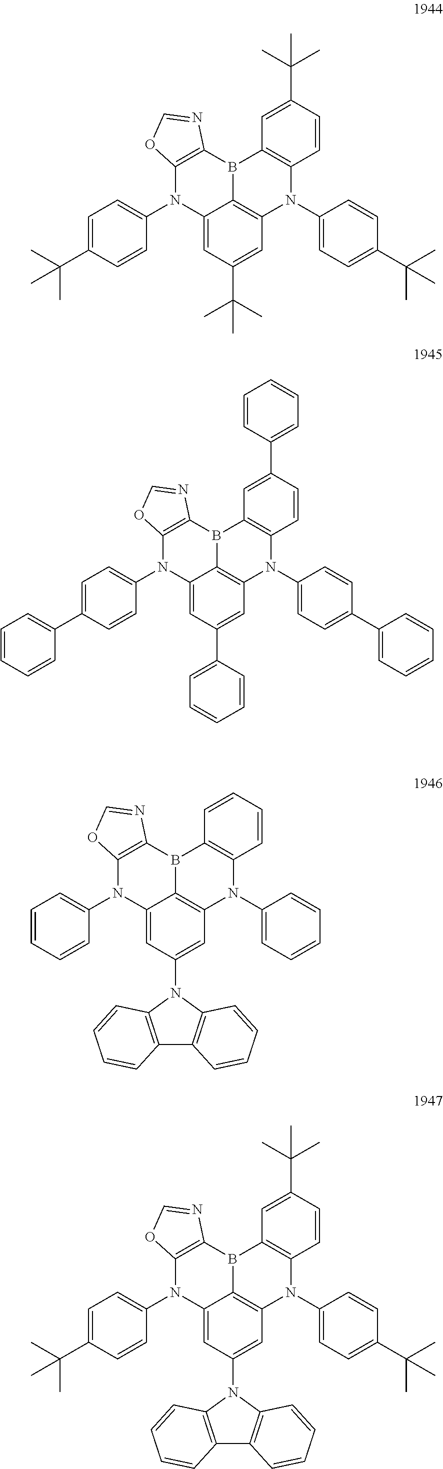

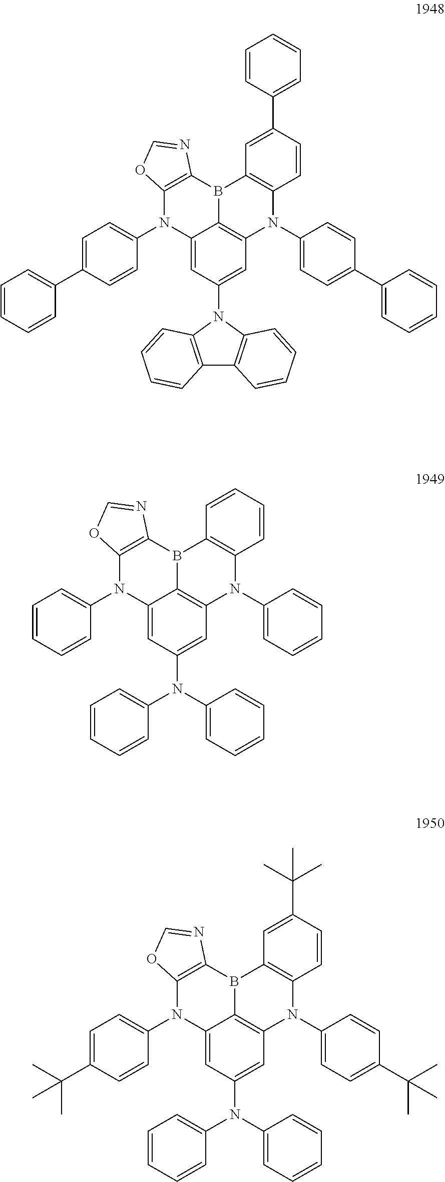 [SMILES: c1ccc(-c2ccc(N3c4ccc(-c5ccccc5)cc4B4c5ncoc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2ocnc2B4c2ccccc2N3c2ccccc2)cc1 ; CC(C)(C)c1ccc(N2c3ccc(C(C)(C)C)cc3B3c4ncoc4N(c4ccc(C(C)(C)C)cc4)c4cc(N(c5ccccc5)c5ccccc5)cc2c43)cc1]

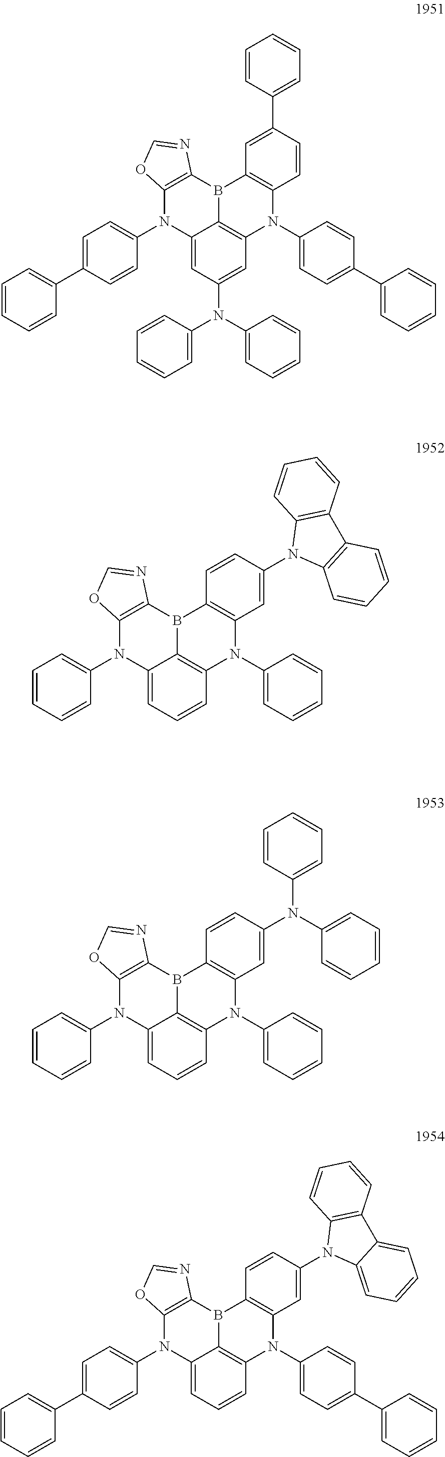 [SMILES: c1ccc(-c2ccc(N3c4ccc(-c5ccccc5)cc4B4c5ncoc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4ncoc4N(c4ccccc4)c4cccc2c43)cc1 ; c1ccc(N(c2ccccc2)c2ccc3c(c2)N(c2ccccc2)c2cccc4c2B3c2ncoc2N4c2ccccc2)cc1 ; c1ccc(-c2ccc(N3c4cc(-n5c6ccccc6c6ccccc65)ccc4B4c5ncoc5N(c5ccc(-c6ccccc6)cc5)c5cccc3c54)cc2)cc1]

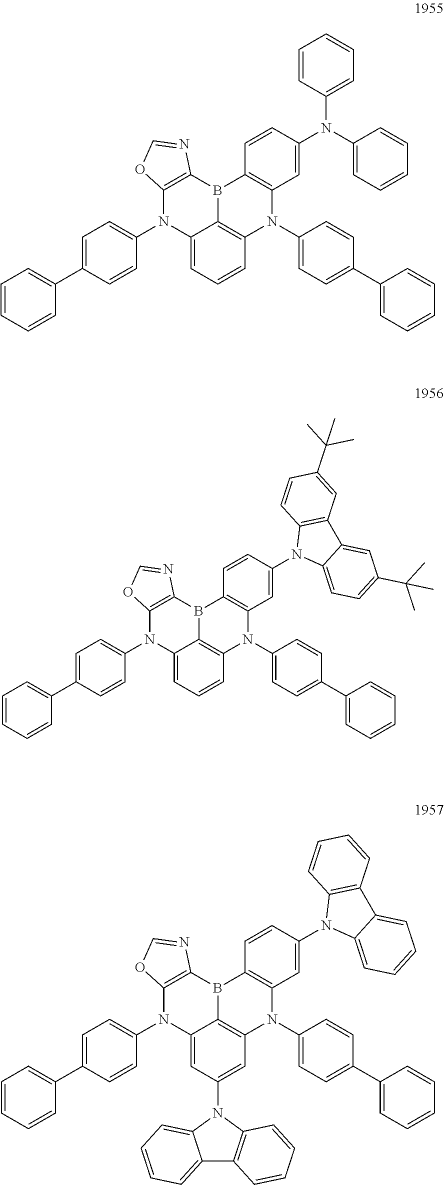 [SMILES: c1ccc(-c2ccc(N3c4cc(N(c5ccccc5)c5ccccc5)ccc4B4c5ncoc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ncoc1N3c1ccc(-c2ccccc2)cc1 ; c1ccc(-c2ccc(N3c4cc(-n5c6ccccc6c6ccccc65)ccc4B4c5ncoc5N(c5ccc(-c6ccccc6)cc5)c5cc(-n6c7ccccc7c7ccccc76)cc3c54)cc2)cc1]

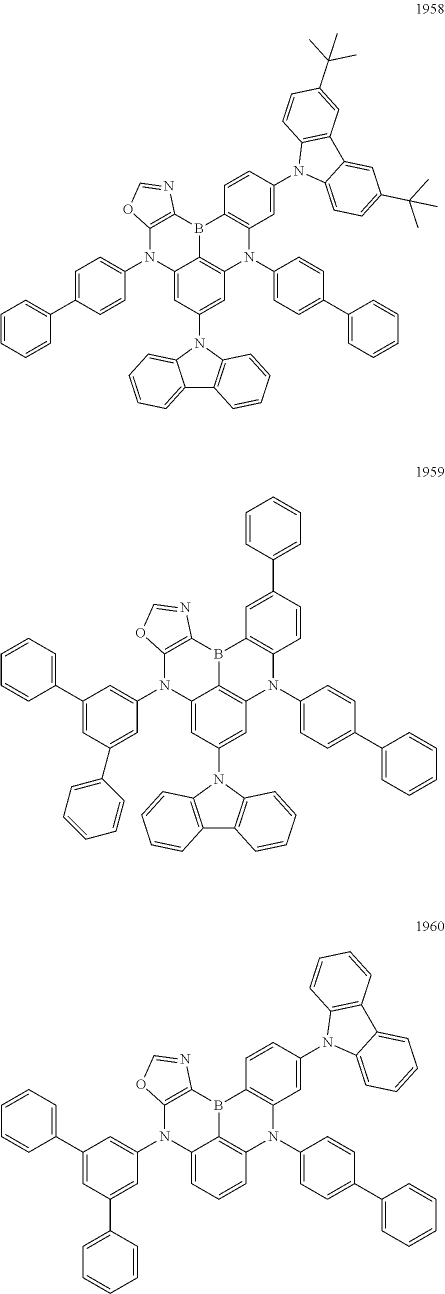 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ncoc1N3c1ccc(-c2ccccc2)cc1 ; c1ccc(-c2ccc(N3c4ccc(-c5ccccc5)cc4B4c5ncoc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ncoc5N(c5cc(-c6ccccc6)cc(-c6ccccc6)c5)c5cccc3c54)cc2)cc1]

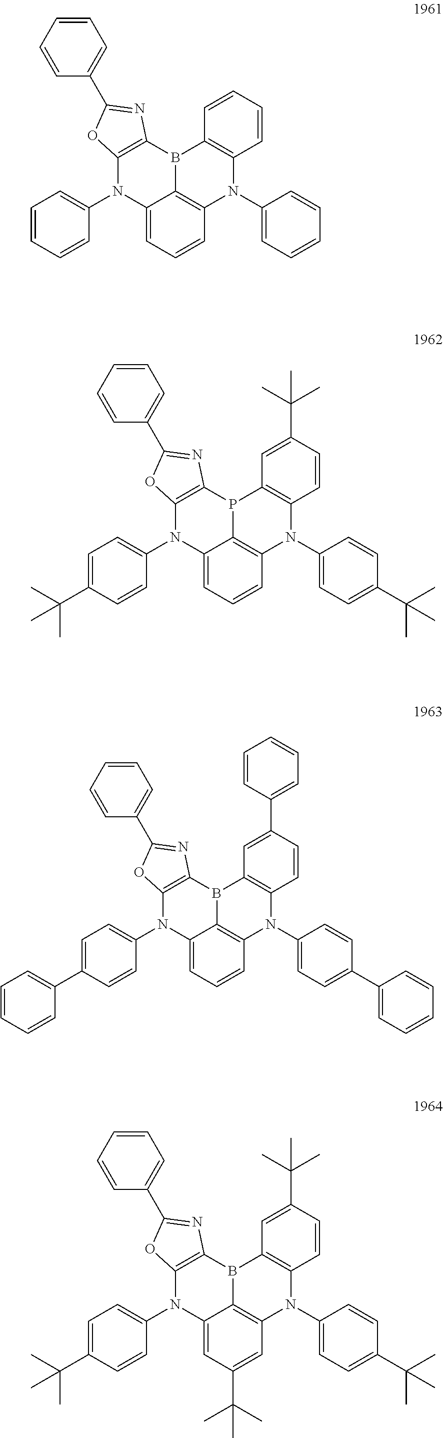

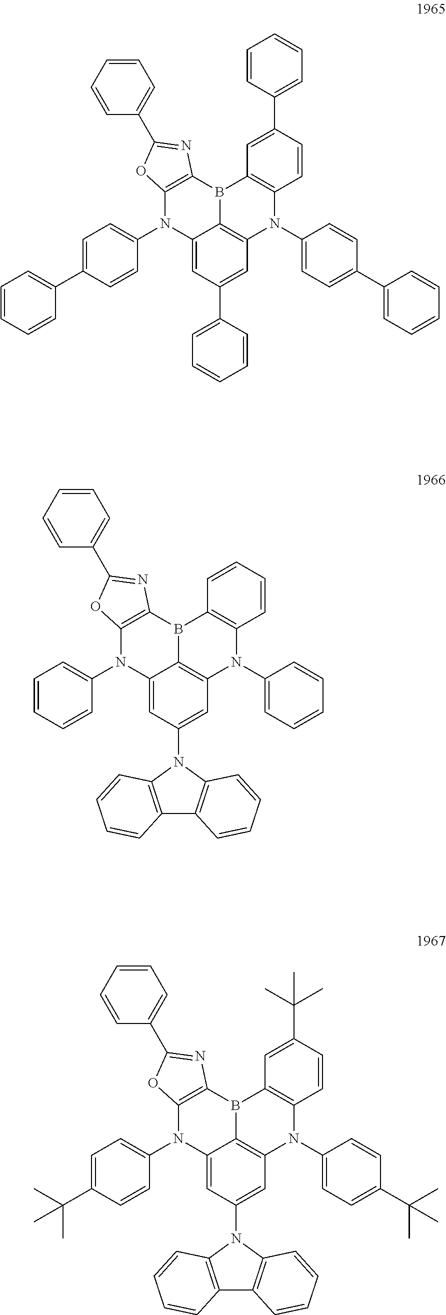 [SMILES: c1ccc(-c2ccc(N3c4ccc(-c5ccccc5)cc4B4c5nc(-c6ccccc6)oc5N(c5ccc(-c6ccccc6)cc5)c5cc(-c6ccccc6)cc3c54)cc2)cc1 ; c1ccc(-c2nc3c(o2)N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4nc(-c5ccccc5)oc4N(c4ccc(C(C)(C)C)cc4)c4cc(-n5c6ccccc6c6ccccc65)cc2c43)cc1]

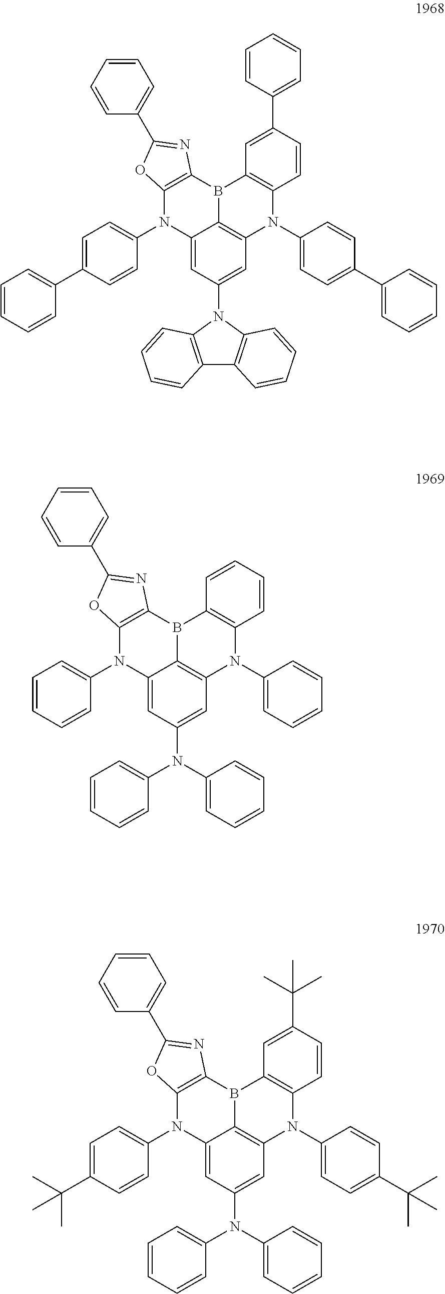 [SMILES: c1ccc(-c2ccc(N3c4ccc(-c5ccccc5)cc4B4c5nc(-c6ccccc6)oc5N(c5ccc(-c6ccccc6)cc5)c5cc(-n6c7ccccc7c7ccccc76)cc3c54)cc2)cc1 ; c1ccc(-c2nc3c(o2)N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4nc(-c5ccccc5)oc4N(c4ccc(C(C)(C)C)cc4)c4cc(N(c5ccccc5)c5ccccc5)cc2c43)cc1]

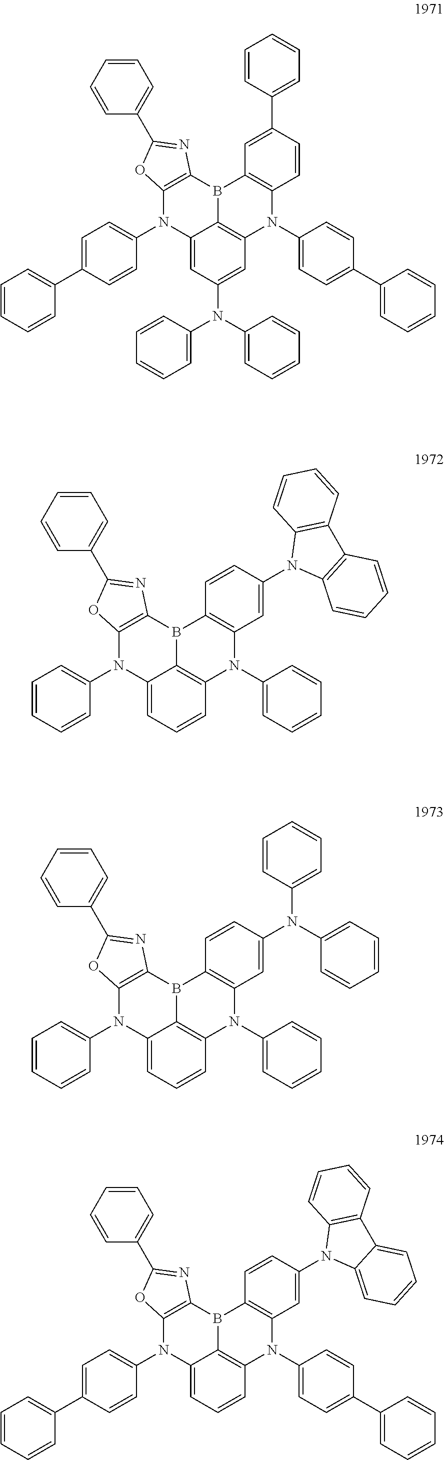 [SMILES: c1ccc(-c2ccc(N3c4ccc(-c5ccccc5)cc4B4c5nc(-c6ccccc6)oc5N(c5ccc(-c6ccccc6)cc5)c5cc(N(c6ccccc6)c6ccccc6)cc3c54)cc2)cc1 ; c1ccc(-c2nc3c(o2)N(c2ccccc2)c2cccc4c2B3c2ccc(-n3c5ccccc5c5ccccc53)cc2N4c2ccccc2)cc1 ; c1ccc(-c2nc3c(o2)N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5nc(-c6ccccc6)oc5N(c5ccc(-c6ccccc6)cc5)c5cccc3c54)cc2)cc1]

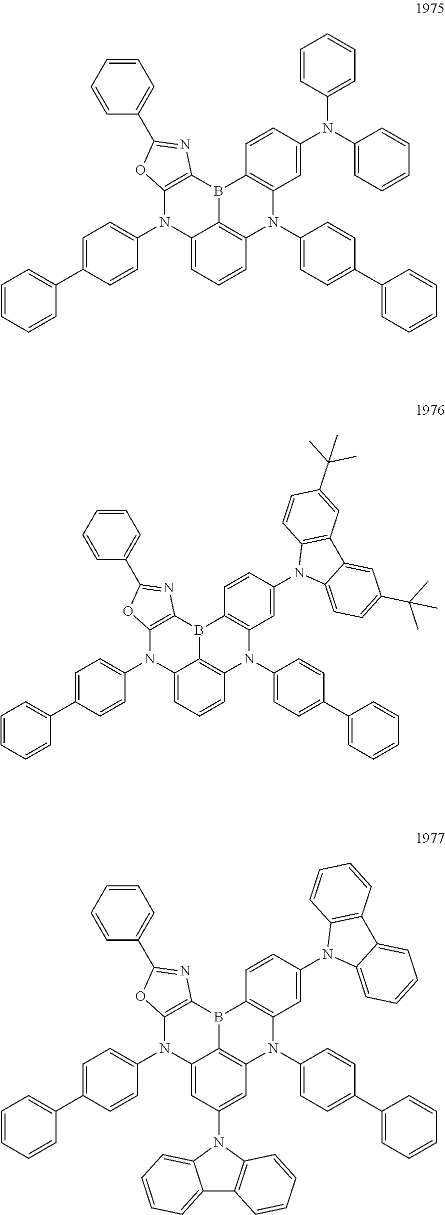 [SMILES: c1ccc(-c2ccc(N3c4cc(N(c5ccccc5)c5ccccc5)ccc4B4c5nc(-c6ccccc6)oc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nc(-c2ccccc2)oc1N3c1ccc(-c2ccccc2)cc1 ; c1ccc(-c2ccc(N3c4cc(-n5c6ccccc6c6ccccc65)ccc4B4c5nc(-c6ccccc6)oc5N(c5ccc(-c6ccccc6)cc5)c5cc(-n6c7ccccc7c7ccccc76)cc3c54)cc2)cc1]

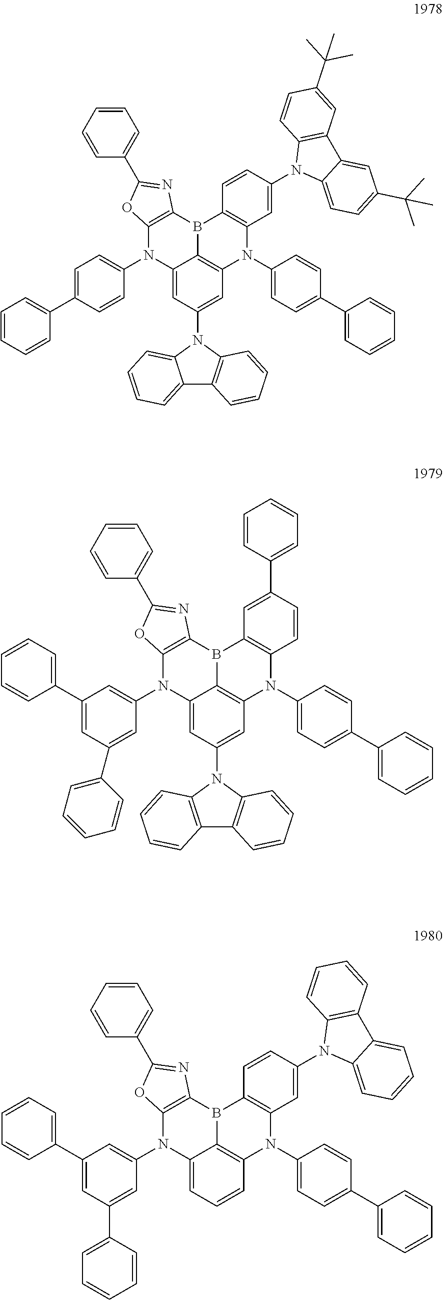 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nc(-c2ccccc2)oc1N3c1ccc(-c2ccccc2)cc1 ; c1ccc(-c2ccc(N3c4ccc(-c5ccccc5)cc4B4c5nc(-c6ccccc6)oc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nc(-c6ccccc6)oc5N(c5cc(-c6ccccc6)cc(-c6ccccc6)c5)c5cccc3c54)cc2)cc1]

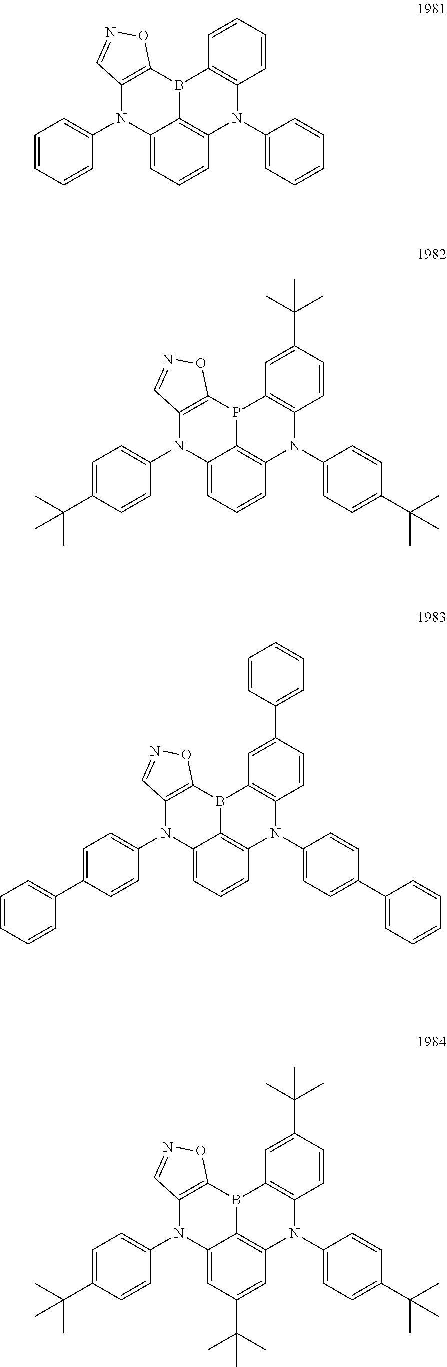

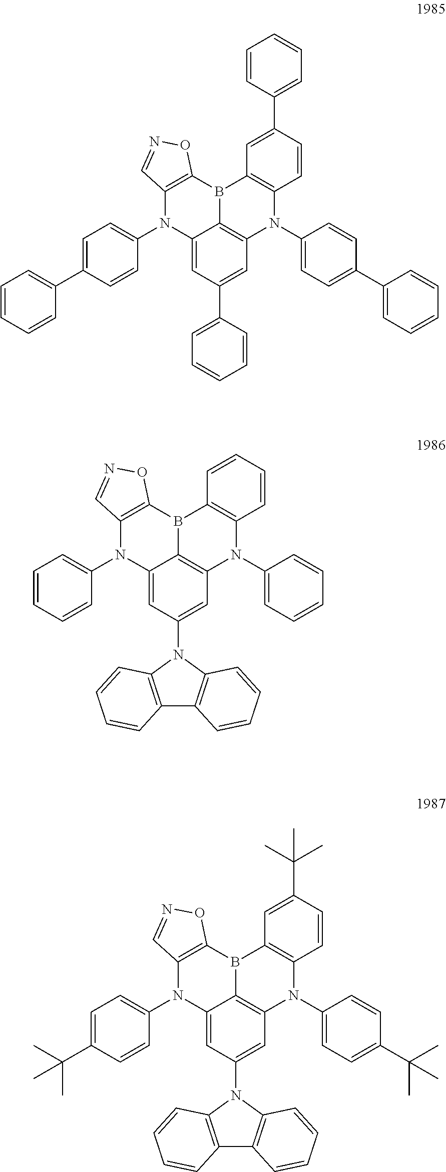 [SMILES: c1ccc(-c2ccc(N3c4ccc(-c5ccccc5)cc4B4c5oncc5N(c5ccc(-c6ccccc6)cc5)c5cc(-c6ccccc6)cc3c54)cc2)cc1 ; c1ccc(N2c3ccccc3B3c4oncc4N(c4ccccc4)c4cc(-n5c6ccccc6c6ccccc65)cc2c43)cc1 ; CC(C)(C)c1ccc(N2c3ccc(C(C)(C)C)cc3B3c4oncc4N(c4ccc(C(C)(C)C)cc4)c4cc(-n5c6ccccc6c6ccccc65)cc2c43)cc1]

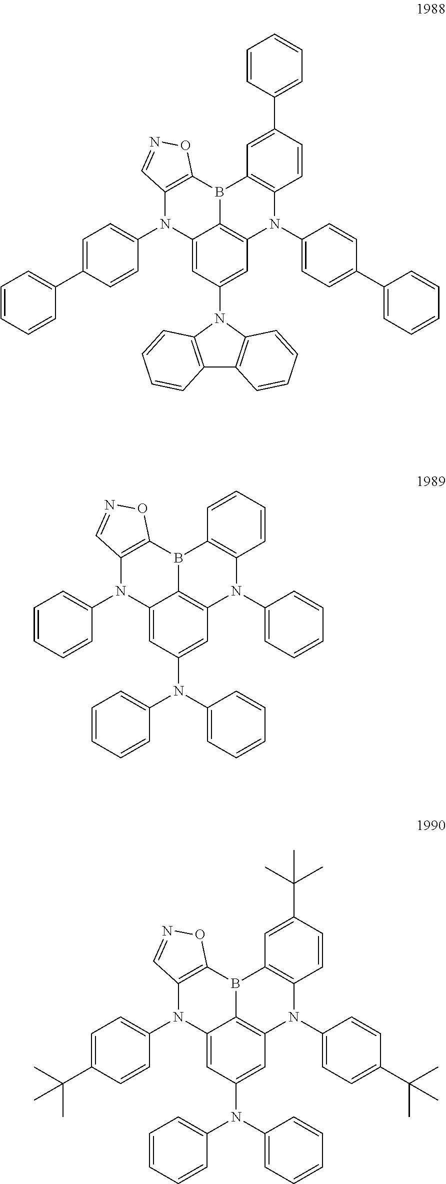 [SMILES: c1ccc(-c2ccc(N3c4ccc(-c5ccccc5)cc4B4c5oncc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2cnoc2B4c2ccccc2N3c2ccccc2)cc1 ; CC(C)(C)c1ccc(N2c3ccc(C(C)(C)C)cc3B3c4oncc4N(c4ccc(C(C)(C)C)cc4)c4cc(N(c5ccccc5)c5ccccc5)cc2c43)cc1]

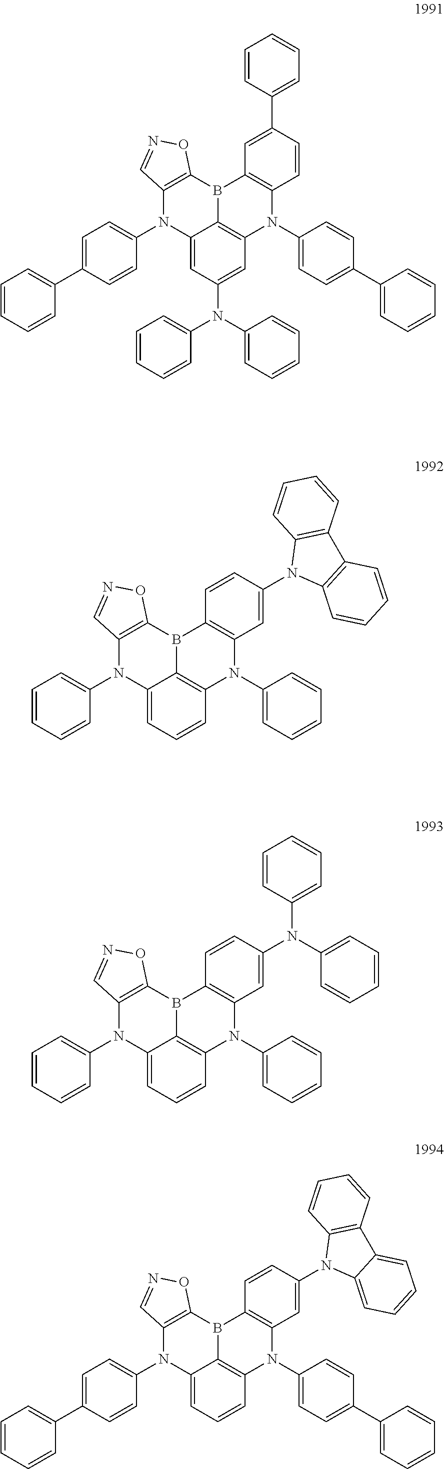 [SMILES: c1ccc(-c2ccc(N3c4ccc(-c5ccccc5)cc4B4c5oncc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4oncc4N(c4ccccc4)c4cccc2c43)cc1 ; c1ccc(N(c2ccccc2)c2ccc3c(c2)N(c2ccccc2)c2cccc4c2B3c2oncc2N4c2ccccc2)cc1 ; c1ccc(-c2ccc(N3c4cc(-n5c6ccccc6c6ccccc65)ccc4B4c5oncc5N(c5ccc(-c6ccccc6)cc5)c5cccc3c54)cc2)cc1]

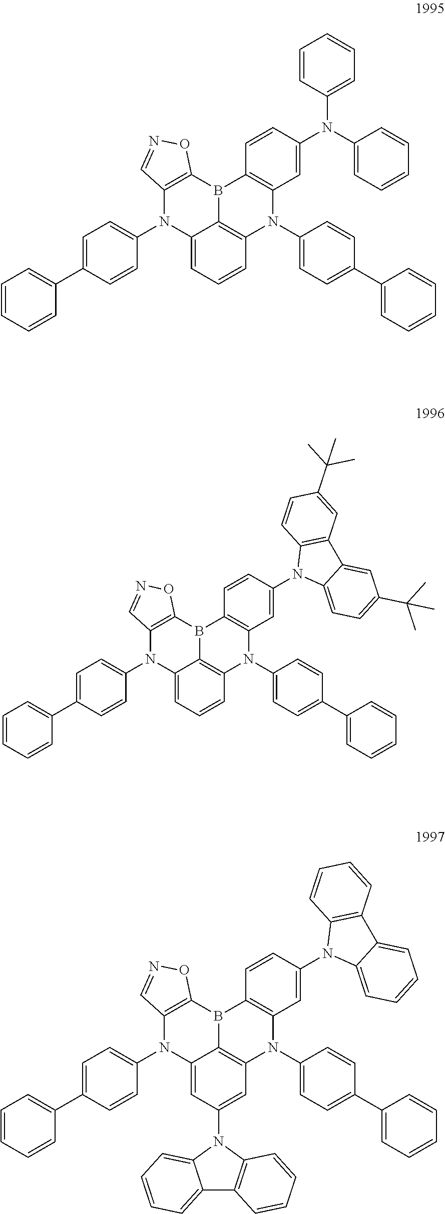 [SMILES: c1ccc(-c2ccc(N3c4cc(N(c5ccccc5)c5ccccc5)ccc4B4c5oncc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1oncc1N3c1ccc(-c2ccccc2)cc1 ; c1ccc(-c2ccc(N3c4cc(-n5c6ccccc6c6ccccc65)ccc4B4c5oncc5N(c5ccc(-c6ccccc6)cc5)c5cc(-n6c7ccccc7c7ccccc76)cc3c54)cc2)cc1]

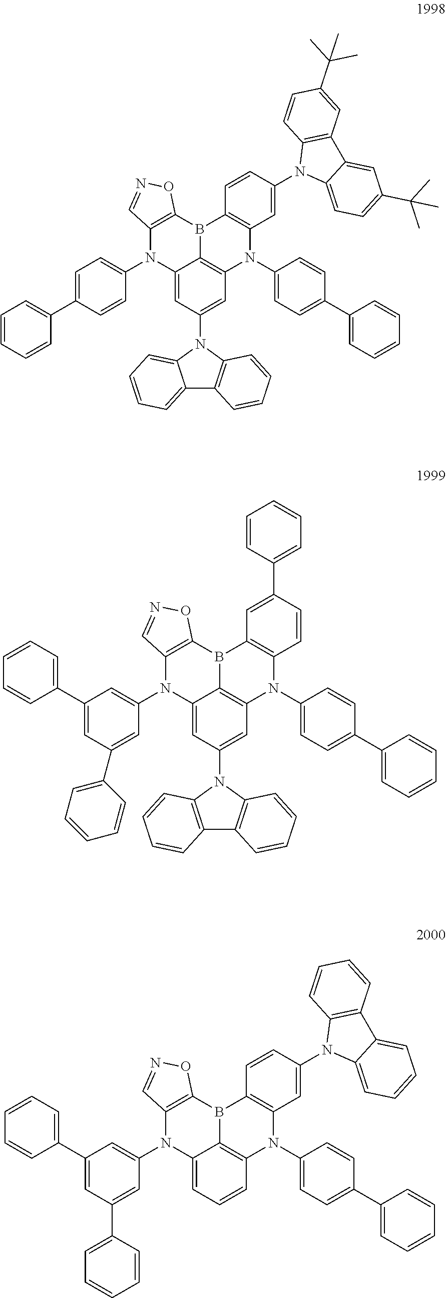 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1oncc1N3c1ccc(-c2ccccc2)cc1 ; c1ccc(-c2ccc(N3c4ccc(-c5ccccc5)cc4B4c5oncc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5oncc5N(c5cc(-c6ccccc6)cc(-c6ccccc6)c5)c5cccc3c54)cc2)cc1]

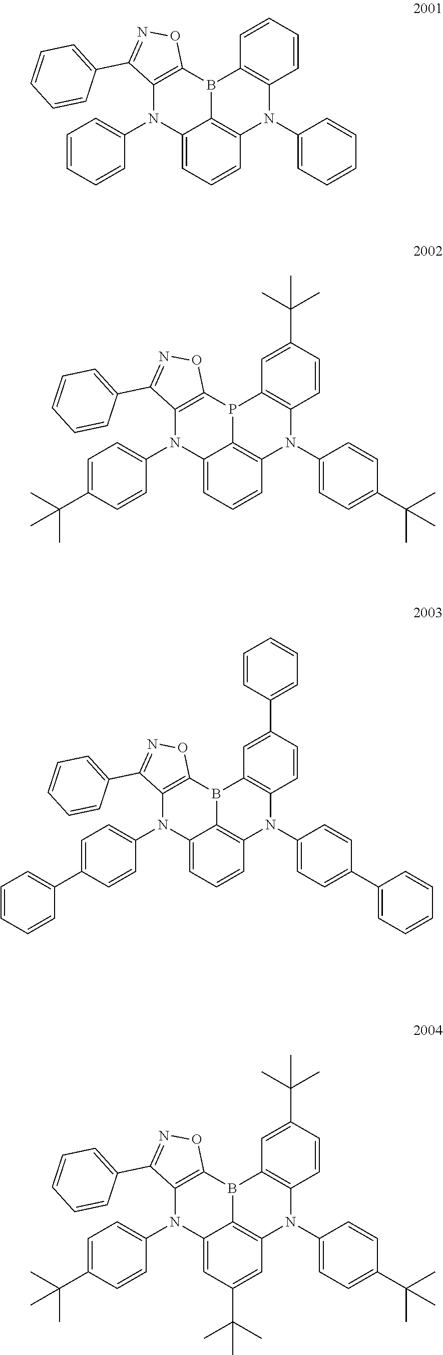

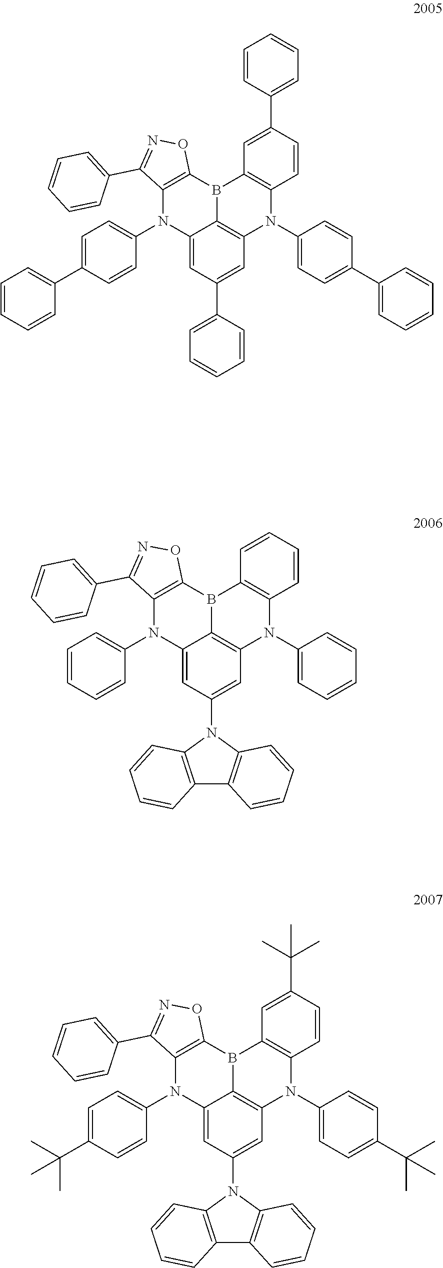 [SMILES: c1ccc(-c2ccc(N3c4ccc(-c5ccccc5)cc4B4c5onc(-c6ccccc6)c5N(c5ccc(-c6ccccc6)cc5)c5cc(-c6ccccc6)cc3c54)cc2)cc1 ; c1ccc(-c2noc3c2N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4onc(-c5ccccc5)c4N(c4ccc(C(C)(C)C)cc4)c4cc(-n5c6ccccc6c6ccccc65)cc2c43)cc1]

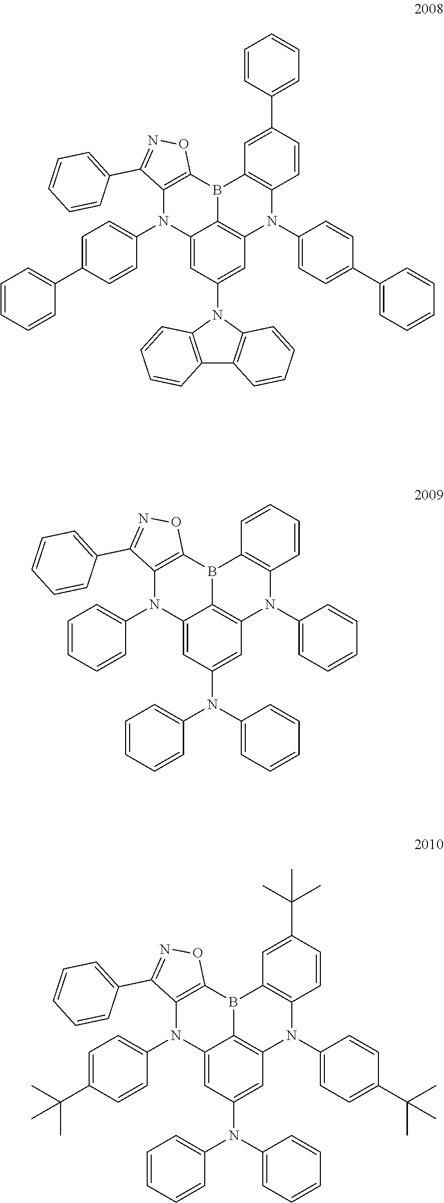 [SMILES: c1ccc(-c2ccc(N3c4ccc(-c5ccccc5)cc4B4c5onc(-c6ccccc6)c5N(c5ccc(-c6ccccc6)cc5)c5cc(-n6c7ccccc7c7ccccc76)cc3c54)cc2)cc1 ; c1ccc(-c2noc3c2N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4onc(-c5ccccc5)c4N(c4ccc(C(C)(C)C)cc4)c4cc(N(c5ccccc5)c5ccccc5)cc2c43)cc1]

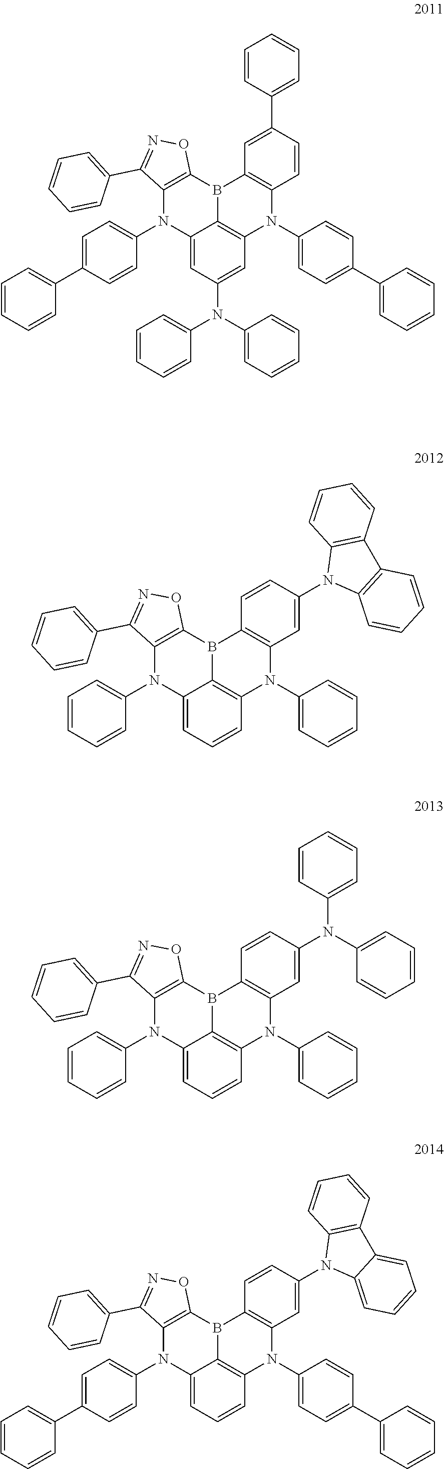 [SMILES: c1ccc(-c2ccc(N3c4ccc(-c5ccccc5)cc4B4c5onc(-c6ccccc6)c5N(c5ccc(-c6ccccc6)cc5)c5cc(N(c6ccccc6)c6ccccc6)cc3c54)cc2)cc1 ; c1ccc(-c2noc3c2N(c2ccccc2)c2cccc4c2B3c2ccc(-n3c5ccccc5c5ccccc53)cc2N4c2ccccc2)cc1 ; c1ccc(-c2noc3c2N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5onc(-c6ccccc6)c5N(c5ccc(-c6ccccc6)cc5)c5cccc3c54)cc2)cc1]

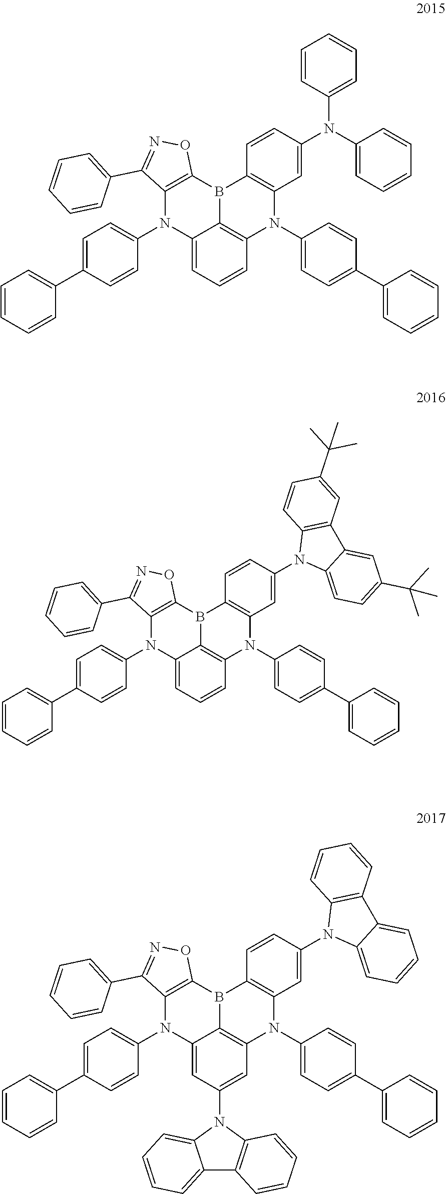 [SMILES: c1ccc(-c2ccc(N3c4cc(N(c5ccccc5)c5ccccc5)ccc4B4c5onc(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1onc(-c2ccccc2)c1N3c1ccc(-c2ccccc2)cc1 ; c1ccc(-c2ccc(N3c4cc(-n5c6ccccc6c6ccccc65)ccc4B4c5onc(-c6ccccc6)c5N(c5ccc(-c6ccccc6)cc5)c5cc(-n6c7ccccc7c7ccccc76)cc3c54)cc2)cc1]

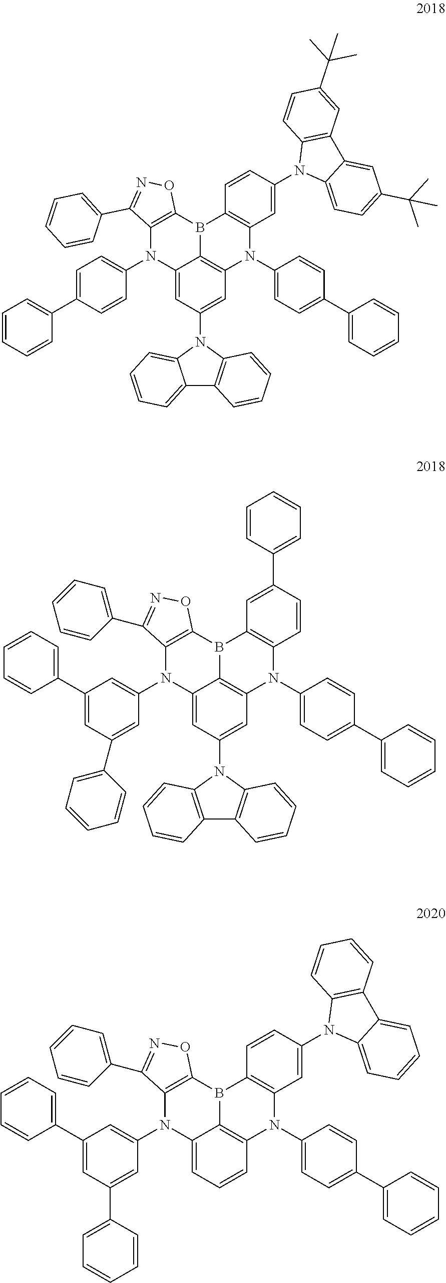 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1onc(-c2ccccc2)c1N3c1ccc(-c2ccccc2)cc1 ; c1ccc(-c2ccc(N3c4ccc(-c5ccccc5)cc4B4c5onc(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5onc(-c6ccccc6)c5N(c5cc(-c6ccccc6)cc(-c6ccccc6)c5)c5cccc3c54)cc2)cc1]

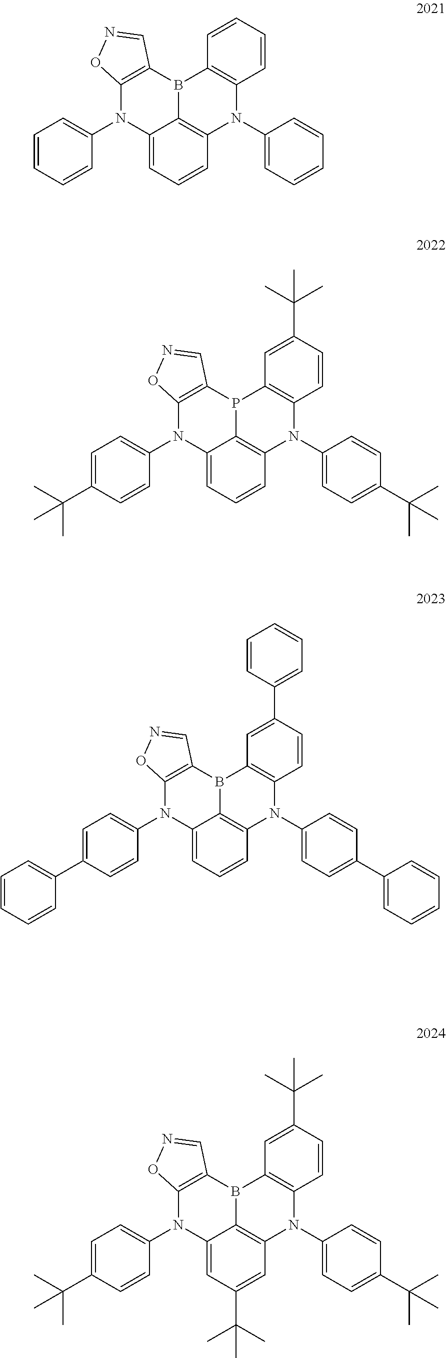

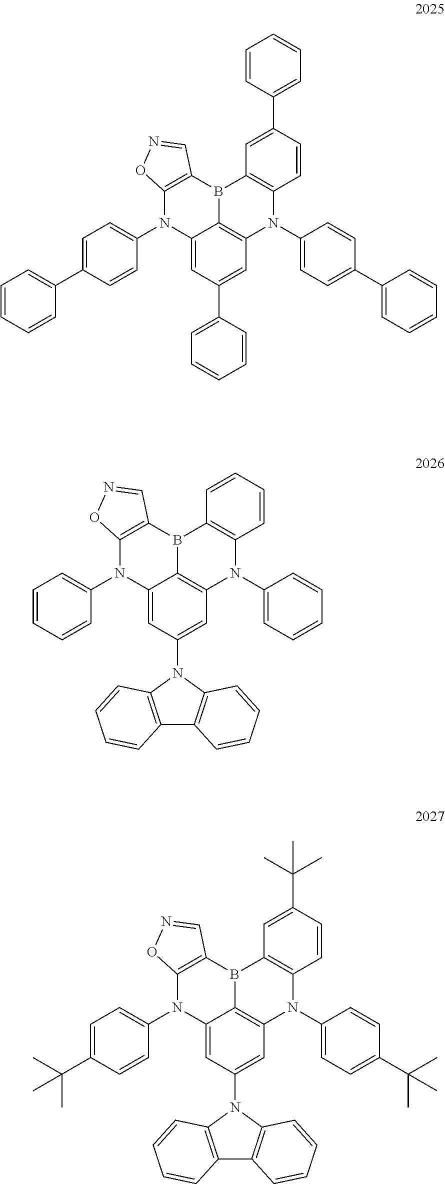 [SMILES: c1ccc(-c2ccc(N3c4ccc(-c5ccccc5)cc4B4c5cnoc5N(c5ccc(-c6ccccc6)cc5)c5cc(-c6ccccc6)cc3c54)cc2)cc1 ; c1ccc(N2c3ccccc3B3c4cnoc4N(c4ccccc4)c4cc(-n5c6ccccc6c6ccccc65)cc2c43)cc1 ; CC(C)(C)c1ccc(N2c3ccc(C(C)(C)C)cc3B3c4cnoc4N(c4ccc(C(C)(C)C)cc4)c4cc(-n5c6ccccc6c6ccccc65)cc2c43)cc1]

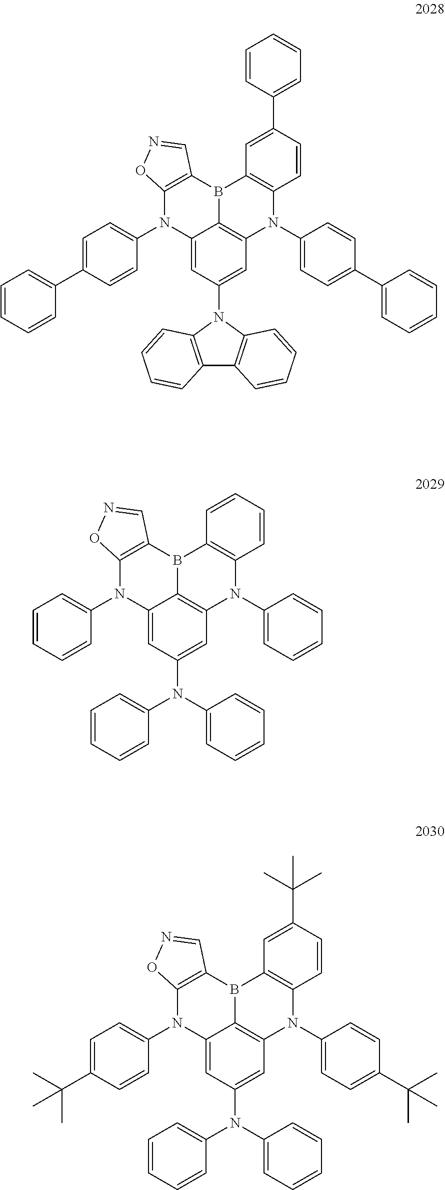 [SMILES: c1ccc(-c2ccc(N3c4ccc(-c5ccccc5)cc4B4c5cnoc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2oncc2B4c2ccccc2N3c2ccccc2)cc1 ; CC(C)(C)c1ccc(N2c3ccc(C(C)(C)C)cc3B3c4cnoc4N(c4ccc(C(C)(C)C)cc4)c4cc(N(c5ccccc5)c5ccccc5)cc2c43)cc1]

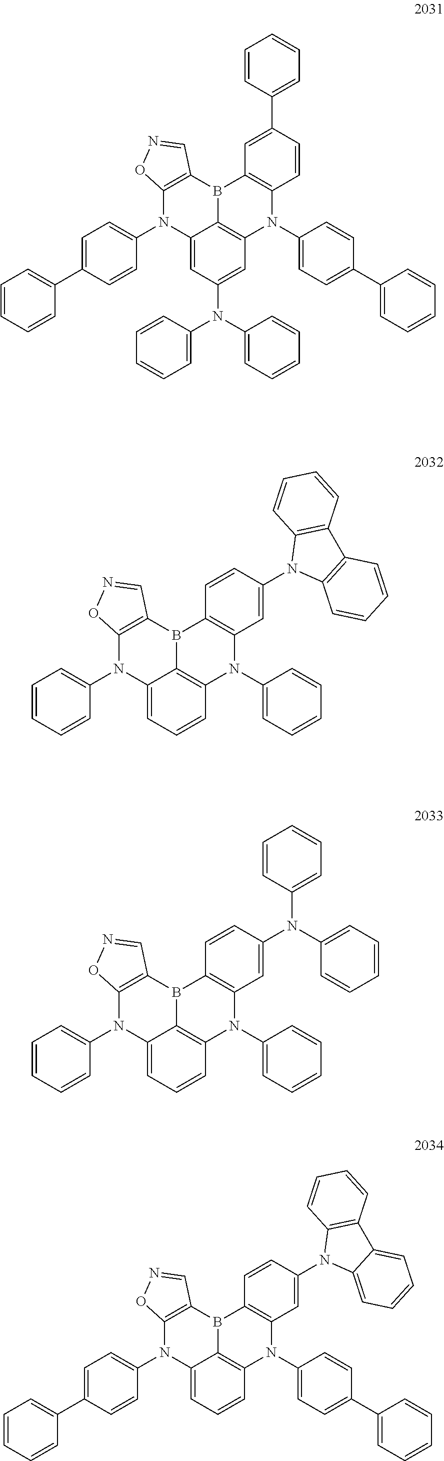 [SMILES: c1ccc(-c2ccc(N3c4ccc(-c5ccccc5)cc4B4c5cnoc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4cnoc4N(c4ccccc4)c4cccc2c43)cc1 ; c1ccc(N(c2ccccc2)c2ccc3c(c2)N(c2ccccc2)c2cccc4c2B3c2cnoc2N4c2ccccc2)cc1 ; c1ccc(-c2ccc(N3c4cc(-n5c6ccccc6c6ccccc65)ccc4B4c5cnoc5N(c5ccc(-c6ccccc6)cc5)c5cccc3c54)cc2)cc1]

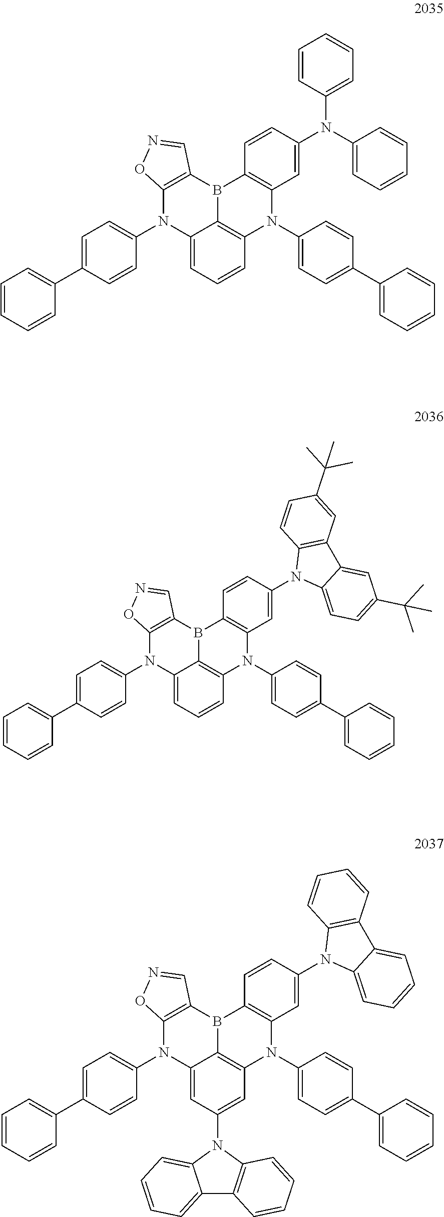 [SMILES: c1ccc(-c2ccc(N3c4cc(N(c5ccccc5)c5ccccc5)ccc4B4c5cnoc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1cnoc1N3c1ccc(-c2ccccc2)cc1 ; c1ccc(-c2ccc(N3c4cc(-n5c6ccccc6c6ccccc65)ccc4B4c5cnoc5N(c5ccc(-c6ccccc6)cc5)c5cc(-n6c7ccccc7c7ccccc76)cc3c54)cc2)cc1]

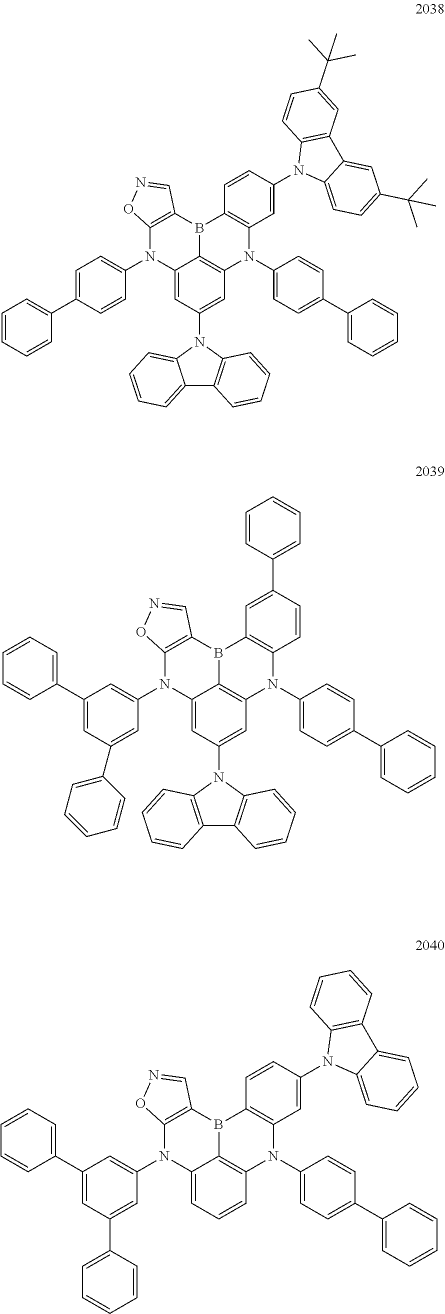 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1cnoc1N3c1ccc(-c2ccccc2)cc1 ; c1ccc(-c2ccc(N3c4ccc(-c5ccccc5)cc4B4c5cnoc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5cnoc5N(c5cc(-c6ccccc6)cc(-c6ccccc6)c5)c5cccc3c54)cc2)cc1]

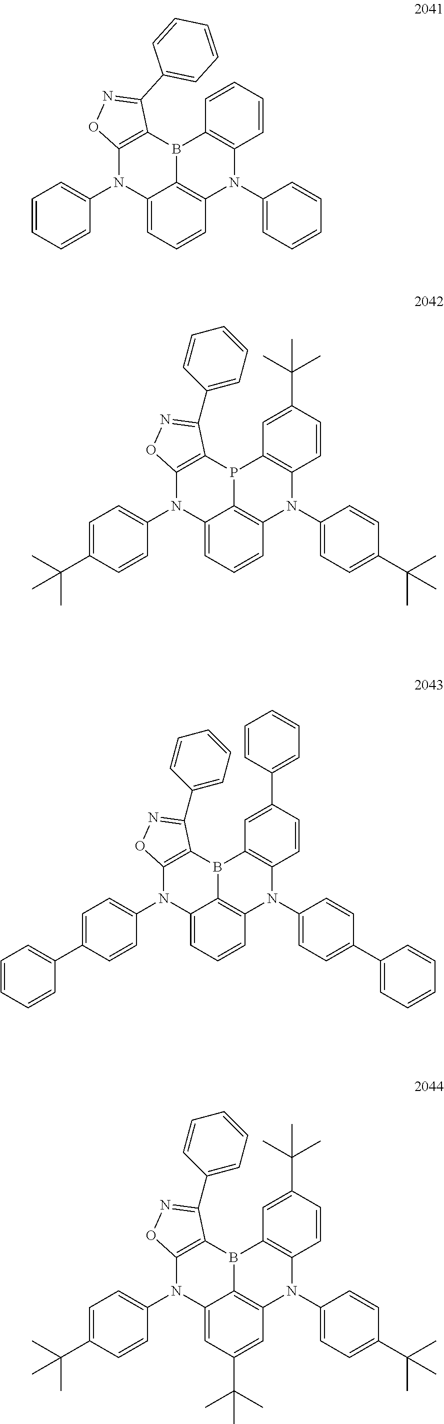

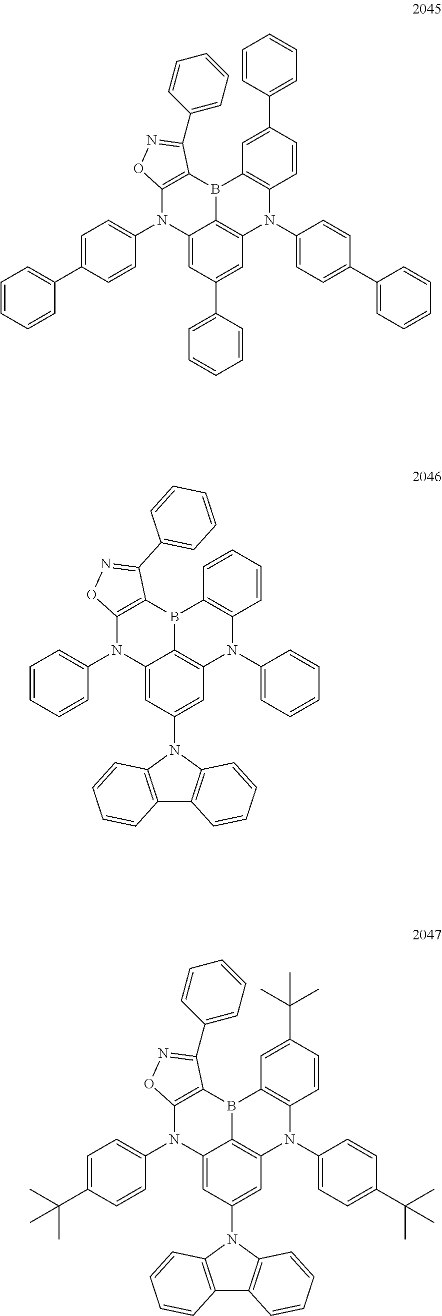 [SMILES: c1ccc(-c2ccc(N3c4ccc(-c5ccccc5)cc4B4c5c3cc(-c3ccccc3)cc5N(c3ccc(-c5ccccc5)cc3)c3onc(-c5ccccc5)c34)cc2)cc1 ; c1ccc(-c2noc3c2B2c4ccccc4N(c4ccccc4)c4cc(-n5c6ccccc6c6ccccc65)cc(c42)N3c2ccccc2)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2onc(-c4ccccc4)c23)cc1]

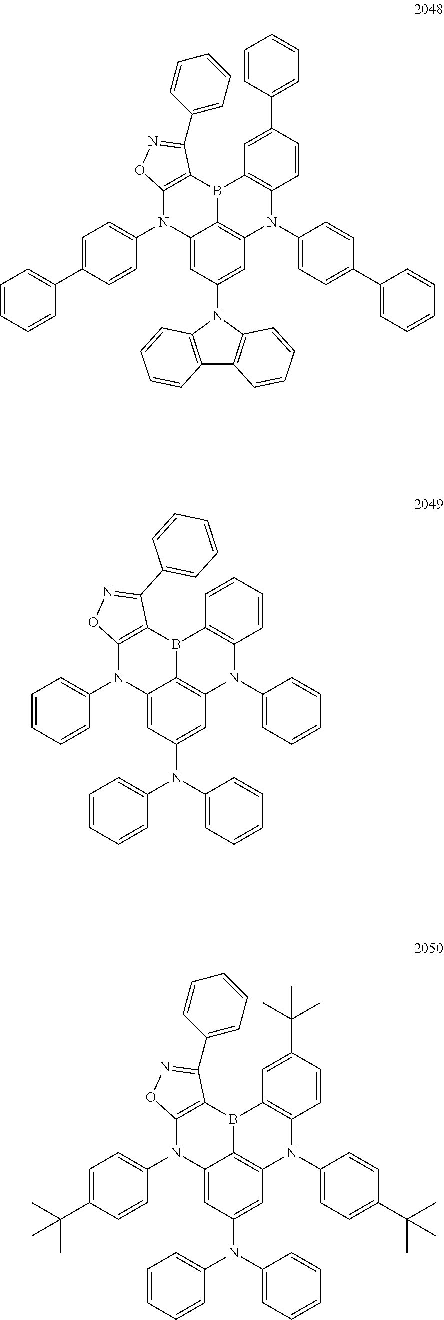 [SMILES: c1ccc(-c2ccc(N3c4ccc(-c5ccccc5)cc4B4c5c3cc(-n3c6ccccc6c6ccccc63)cc5N(c3ccc(-c5ccccc5)cc3)c3onc(-c5ccccc5)c34)cc2)cc1 ; c1ccc(-c2noc3c2B2c4ccccc4N(c4ccccc4)c4cc(N(c5ccccc5)c5ccccc5)cc(c42)N3c2ccccc2)cc1 ; CC(C)(C)c1ccc(N2c3ccc(C(C)(C)C)cc3B3c4c2cc(N(c2ccccc2)c2ccccc2)cc4N(c2ccc(C(C)(C)C)cc2)c2onc(-c4ccccc4)c23)cc1]

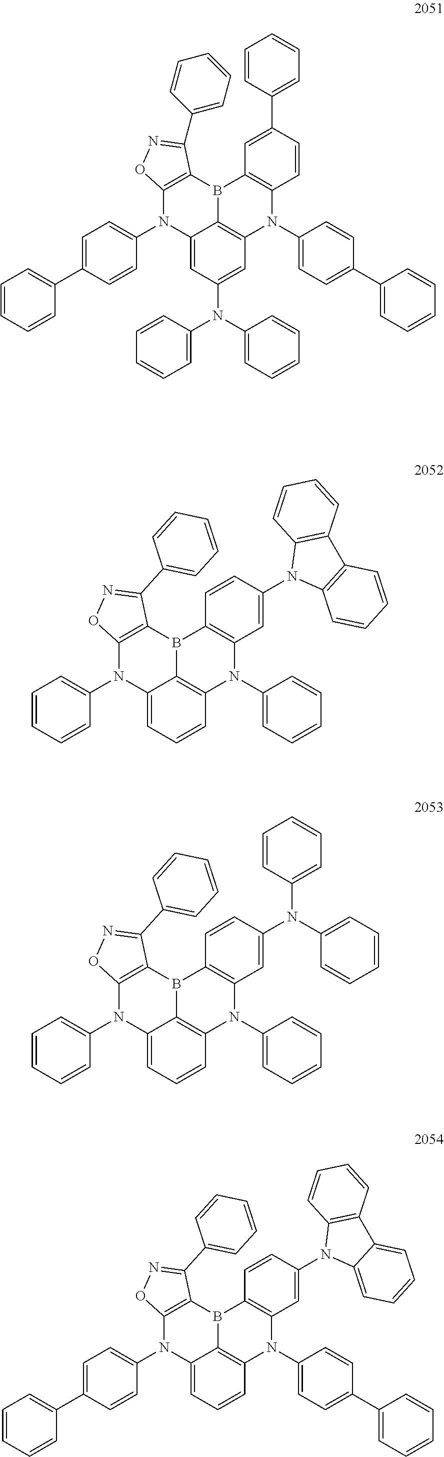 [SMILES: c1ccc(-c2ccc(N3c4ccc(-c5ccccc5)cc4B4c5c3cc(N(c3ccccc3)c3ccccc3)cc5N(c3ccc(-c5ccccc5)cc3)c3onc(-c5ccccc5)c34)cc2)cc1 ; c1ccc(-c2noc3c2B2c4ccc(-n5c6ccccc6c6ccccc65)cc4N(c4ccccc4)c4cccc(c42)N3c2ccccc2)cc1 ; c1ccc(-c2noc3c2B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5c3cccc5N(c3ccc(-c5ccccc5)cc3)c3onc(-c5ccccc5)c34)cc2)cc1]

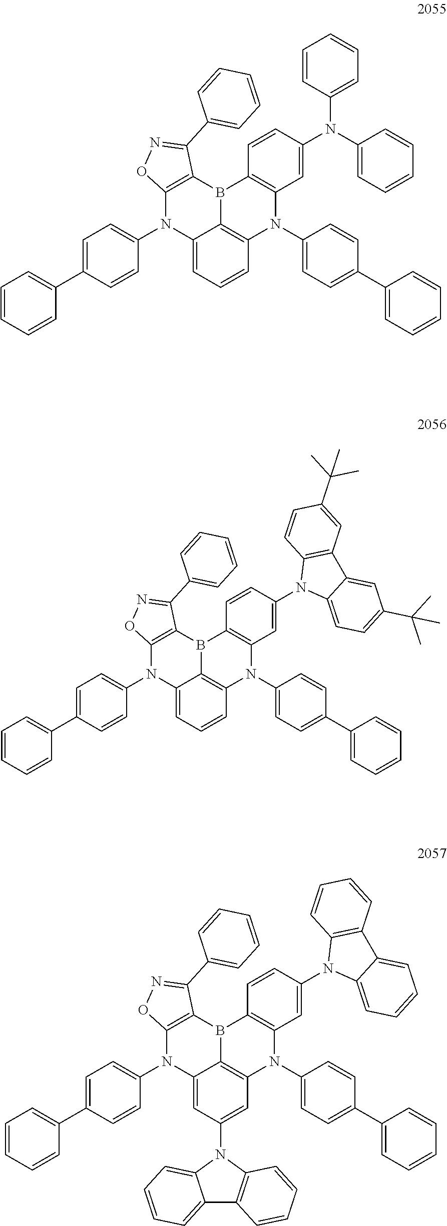 [SMILES: c1ccc(-c2ccc(N3c4cc(N(c5ccccc5)c5ccccc5)ccc4B4c5c3cccc5N(c3ccc(-c5ccccc5)cc3)c3onc(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(-c2ccccc2)noc1N3c1ccc(-c2ccccc2)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3onc(-c5ccccc5)c34)cc2)cc1]

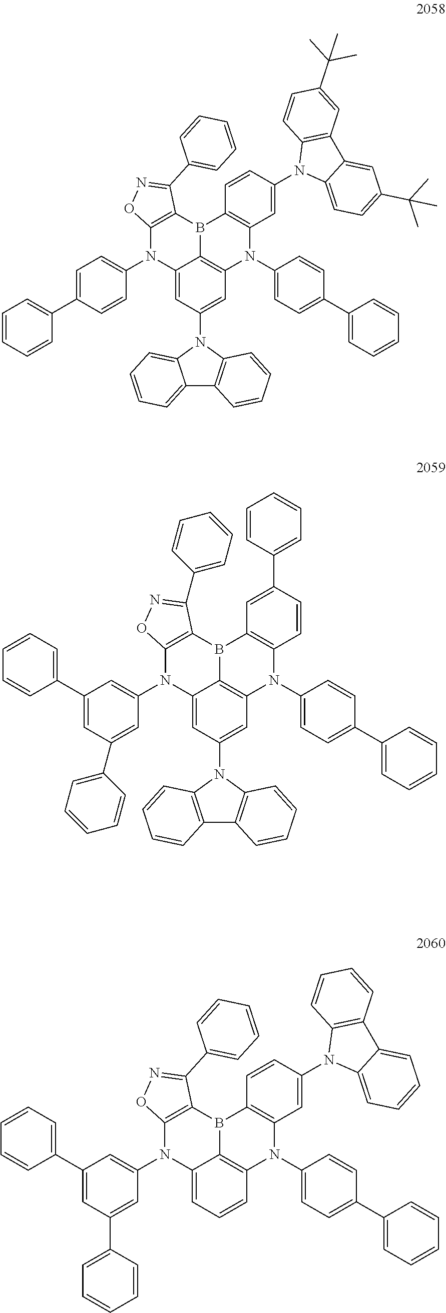 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(-c2ccccc2)noc1N3c1ccc(-c2ccccc2)cc1 ; c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3onc(-c5ccccc5)c34)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3onc(-c5ccccc5)c34)cc2)cc1]

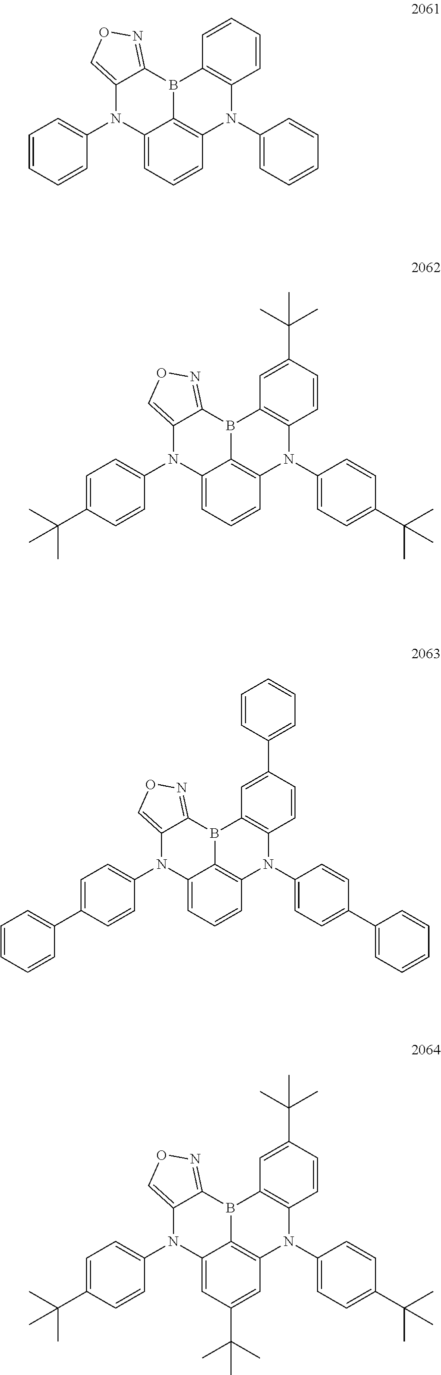

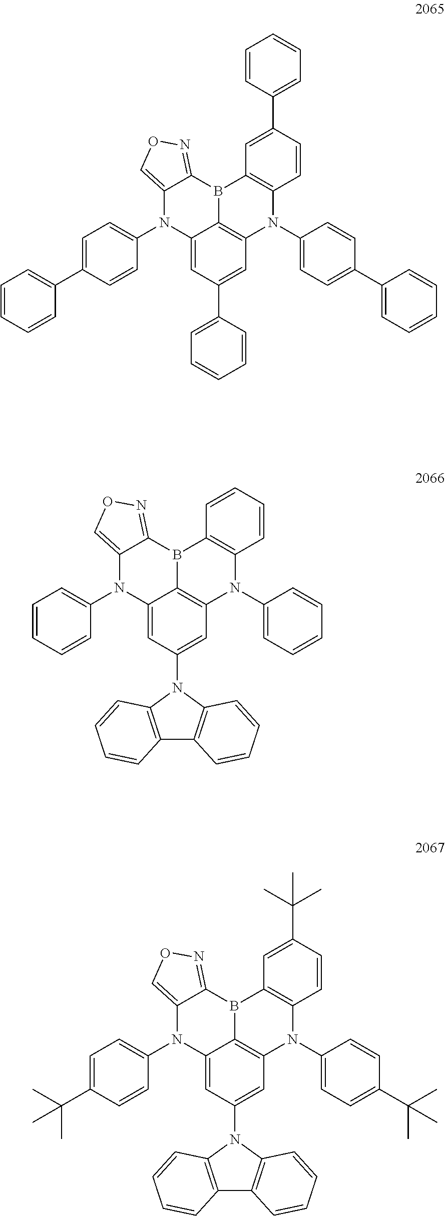 [SMILES: c1ccc(-c2ccc(N3c4ccc(-c5ccccc5)cc4B4c5nocc5N(c5ccc(-c6ccccc6)cc5)c5cc(-c6ccccc6)cc3c54)cc2)cc1 ; c1ccc(N2c3ccccc3B3c4nocc4N(c4ccccc4)c4cc(-n5c6ccccc6c6ccccc65)cc2c43)cc1 ; CC(C)(C)c1ccc(N2c3ccc(C(C)(C)C)cc3B3c4nocc4N(c4ccc(C(C)(C)C)cc4)c4cc(-n5c6ccccc6c6ccccc65)cc2c43)cc1]

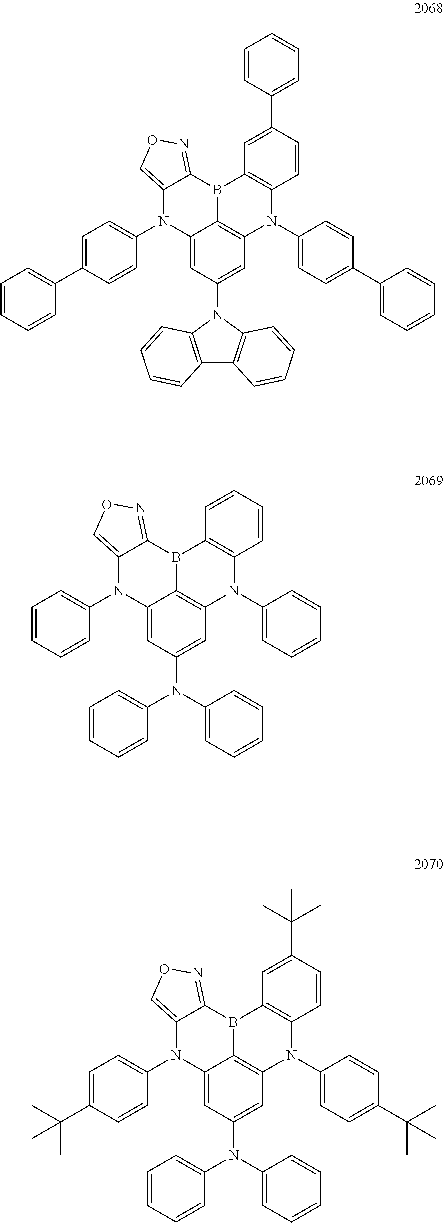 [SMILES: c1ccc(-c2ccc(N3c4ccc(-c5ccccc5)cc4B4c5nocc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2conc2B4c2ccccc2N3c2ccccc2)cc1 ; CC(C)(C)c1ccc(N2c3ccc(C(C)(C)C)cc3B3c4nocc4N(c4ccc(C(C)(C)C)cc4)c4cc(N(c5ccccc5)c5ccccc5)cc2c43)cc1]

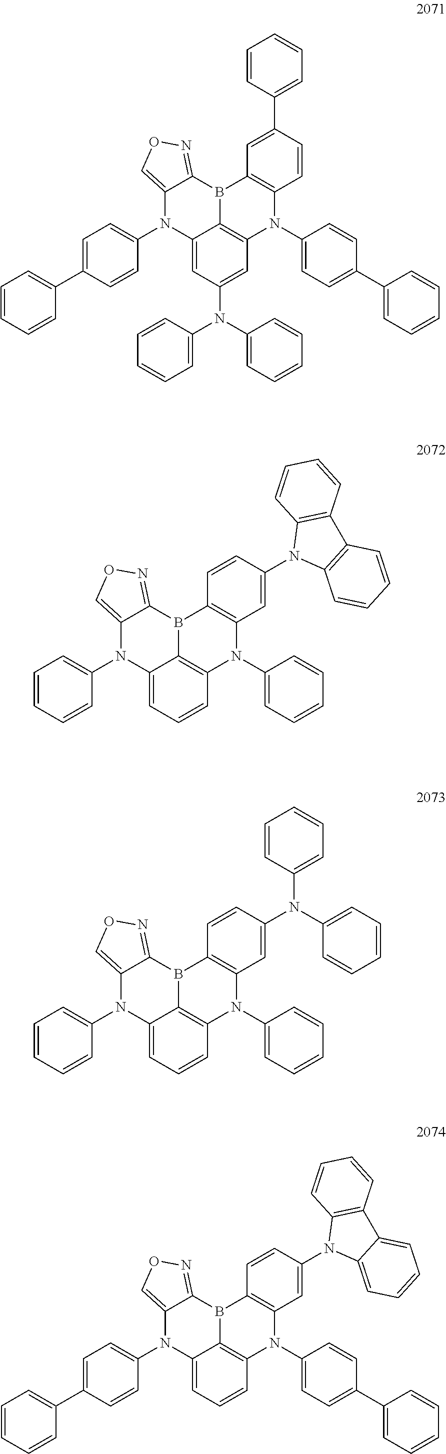 [SMILES: c1ccc(-c2ccc(N3c4ccc(-c5ccccc5)cc4B4c5nocc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nocc4N(c4ccccc4)c4cccc2c43)cc1 ; c1ccc(N(c2ccccc2)c2ccc3c(c2)N(c2ccccc2)c2cccc4c2B3c2nocc2N4c2ccccc2)cc1 ; c1ccc(-c2ccc(N3c4cc(-n5c6ccccc6c6ccccc65)ccc4B4c5nocc5N(c5ccc(-c6ccccc6)cc5)c5cccc3c54)cc2)cc1]

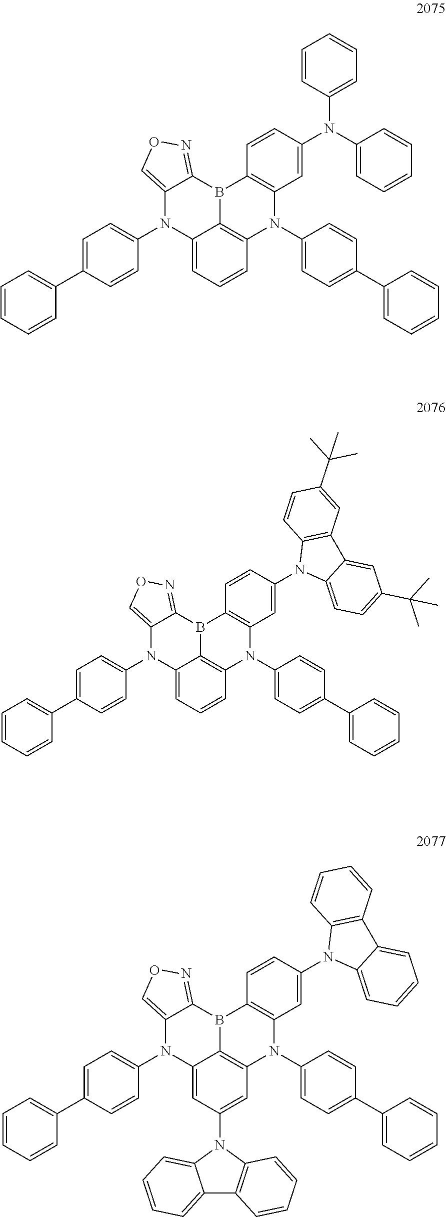 [SMILES: c1ccc(-c2ccc(N3c4cc(N(c5ccccc5)c5ccccc5)ccc4B4c5nocc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nocc1N3c1ccc(-c2ccccc2)cc1 ; c1ccc(-c2ccc(N3c4cc(-n5c6ccccc6c6ccccc65)ccc4B4c5nocc5N(c5ccc(-c6ccccc6)cc5)c5cc(-n6c7ccccc7c7ccccc76)cc3c54)cc2)cc1]

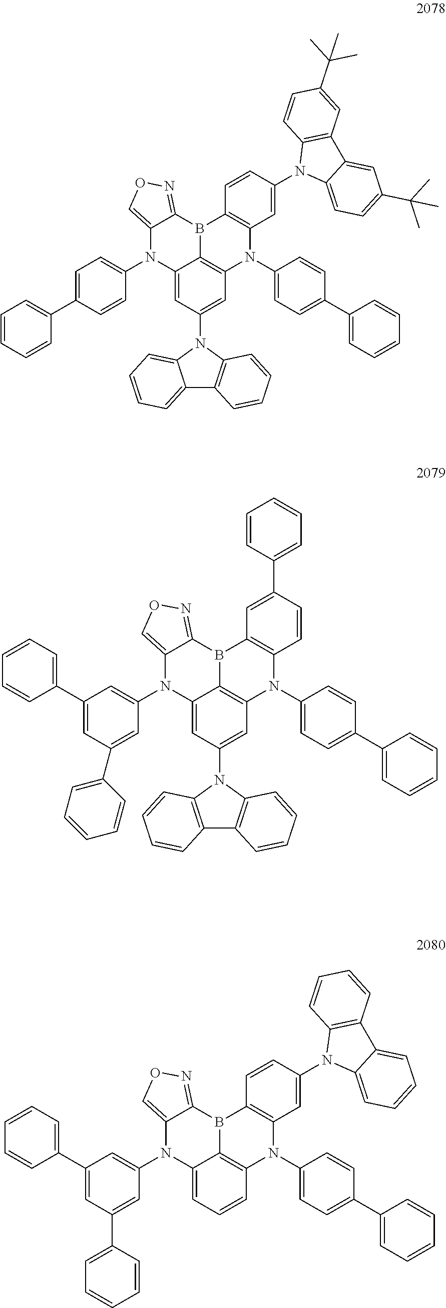 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nocc1N3c1ccc(-c2ccccc2)cc1 ; c1ccc(-c2ccc(N3c4ccc(-c5ccccc5)cc4B4c5nocc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nocc5N(c5cc(-c6ccccc6)cc(-c6ccccc6)c5)c5cccc3c54)cc2)cc1]

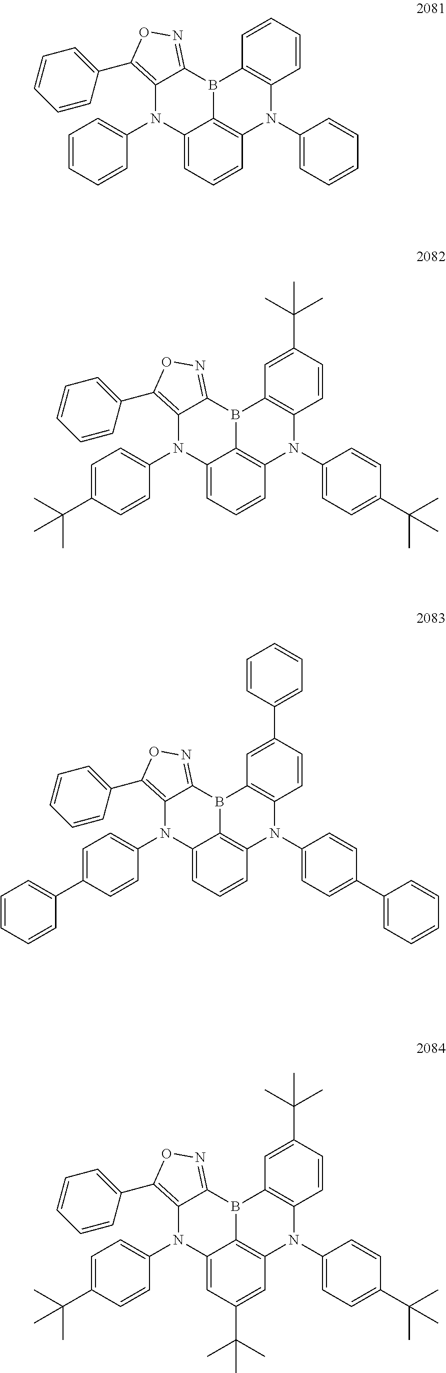

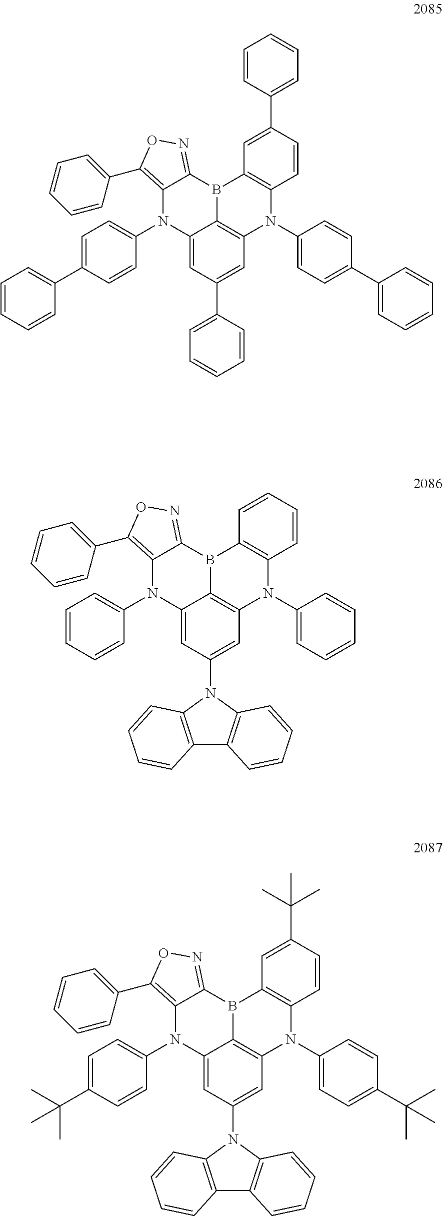 [SMILES: c1ccc(-c2ccc(N3c4ccc(-c5ccccc5)cc4B4c5noc(-c6ccccc6)c5N(c5ccc(-c6ccccc6)cc5)c5cc(-c6ccccc6)cc3c54)cc2)cc1 ; c1ccc(-c2onc3c2N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4noc(-c5ccccc5)c4N(c4ccc(C(C)(C)C)cc4)c4cc(-n5c6ccccc6c6ccccc65)cc2c43)cc1]

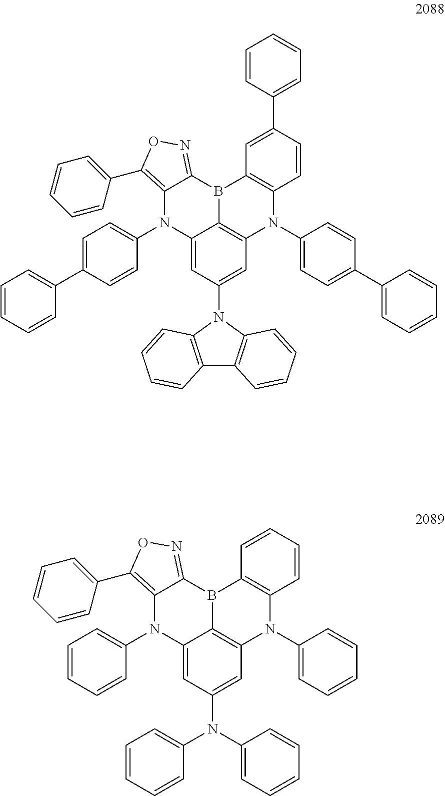 [SMILES: c1ccc(-c2ccc(N3c4ccc(-c5ccccc5)cc4B4c5noc(-c6ccccc6)c5N(c5ccc(-c6ccccc6)cc5)c5cc(-n6c7ccccc7c7ccccc76)cc3c54)cc2)cc1 ; c1ccc(-c2onc3c2N(c2ccccc2)c2cc(N(c4ccccc4)c4ccccc4)cc4c2B3c2ccccc2N4c2ccccc2)cc1]

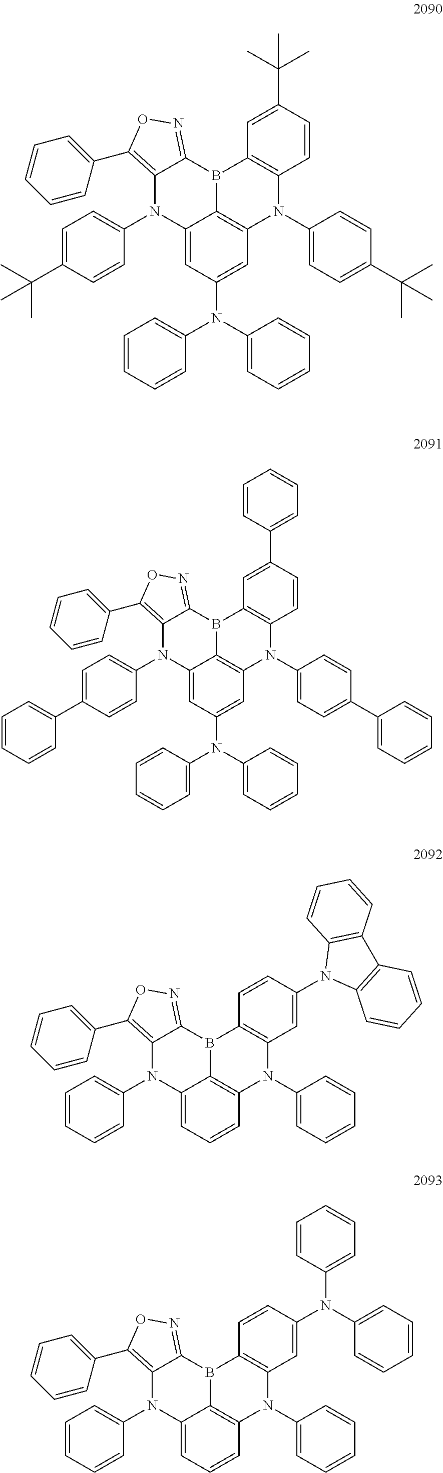 [SMILES: CC(C)(C)c1ccc(N2c3ccc(C(C)(C)C)cc3B3c4noc(-c5ccccc5)c4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5noc(-c6ccccc6)c5N(c5ccc(-c6ccccc6)cc5)c5cc(N(c6ccccc6)c6ccccc6)cc3c54)cc2)cc1 ; c1ccc(-c2onc3c2N(c2ccccc2)c2cccc4c2B3c2ccc(-n3c5ccccc5c5ccccc53)cc2N4c2ccccc2)cc1 ; c1ccc(-c2onc3c2N(c2ccccc2)c2cccc4c2B3c2ccc(N(c3ccccc3)c3ccccc3)cc2N4c2ccccc2)cc1]

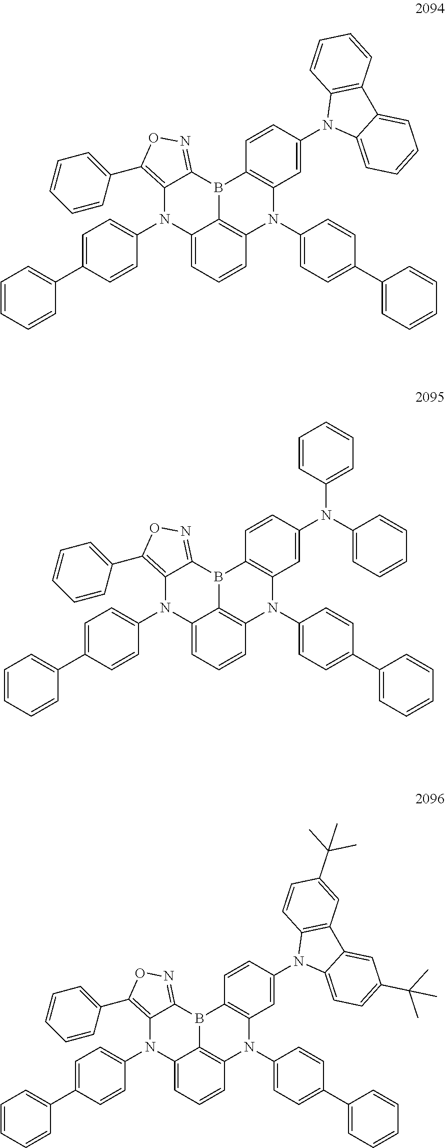 [SMILES: c1ccc(-c2ccc(N3c4cc(-n5c6ccccc6c6ccccc65)ccc4B4c5noc(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5noc(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1noc(-c2ccccc2)c1N3c1ccc(-c2ccccc2)cc1]

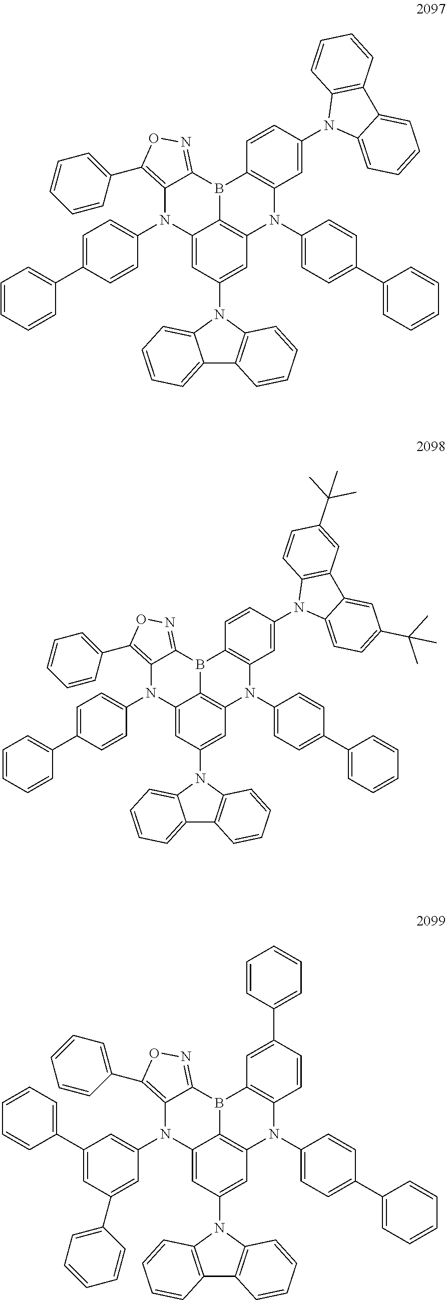 [SMILES: c1ccc(-c2ccc(N3c4cc(-n5c6ccccc6c6ccccc65)ccc4B4c5noc(-c6ccccc6)c5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1noc(-c2ccccc2)c1N3c1ccc(-c2ccccc2)cc1 ; c1ccc(-c2ccc(N3c4ccc(-c5ccccc5)cc4B4c5noc(-c6ccccc6)c5N(c5cc(-c6ccccc6)cc(-c6ccccc6)c5)c5cc(-n6c7ccccc7c7ccccc76)cc3c54)cc2)cc1]

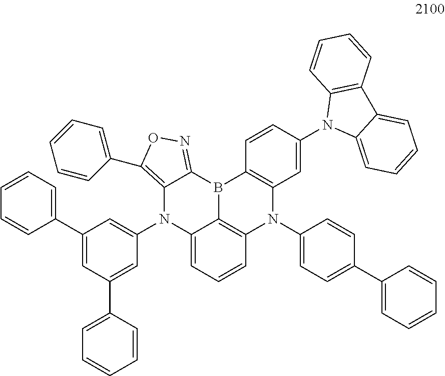 [SMILES: c1ccc(-c2ccc(N3c4cc(-n5c6ccccc6c6ccccc65)ccc4B4c5noc(-c6ccccc6)c5N(c5cc(-c6ccccc6)cc(-c6ccccc6)c5)c5cccc3c54)cc2)cc1]

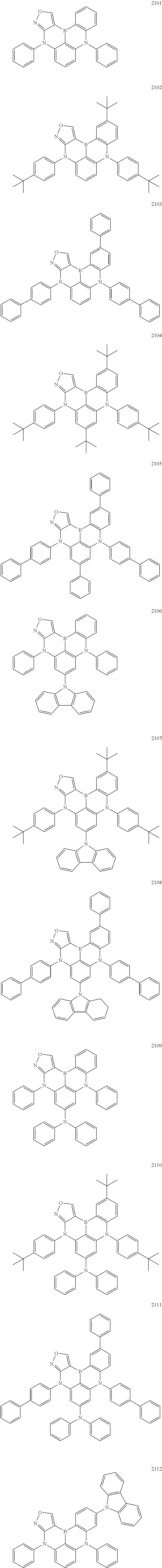

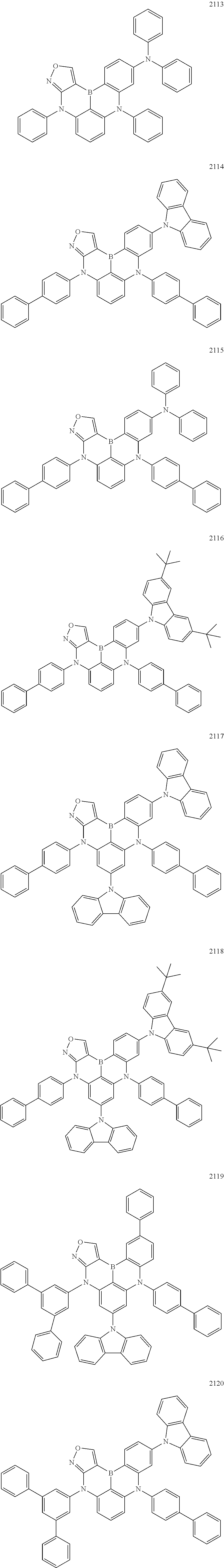

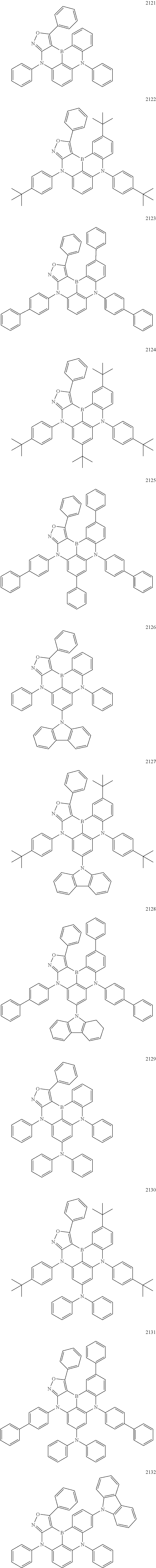

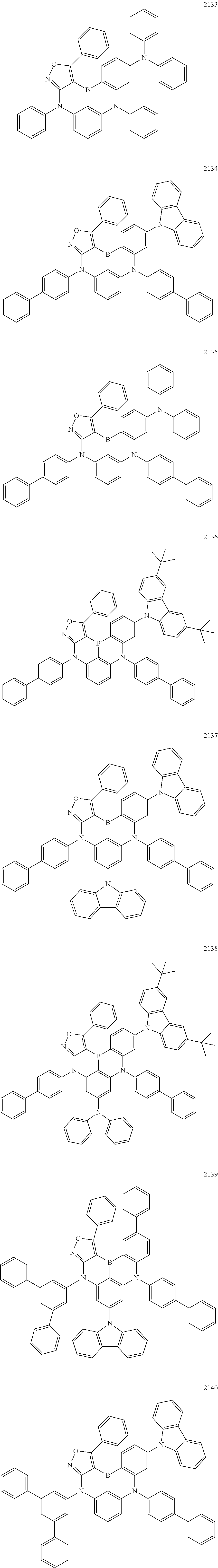

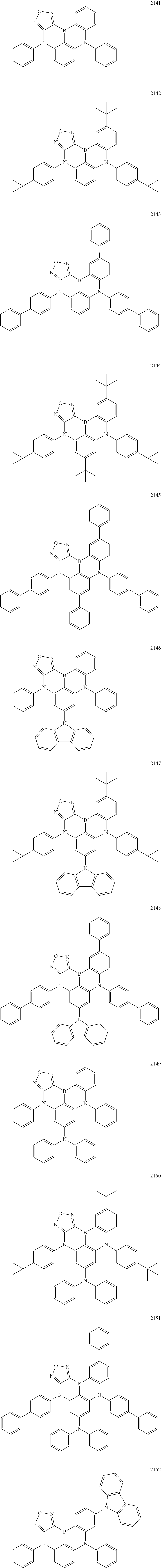

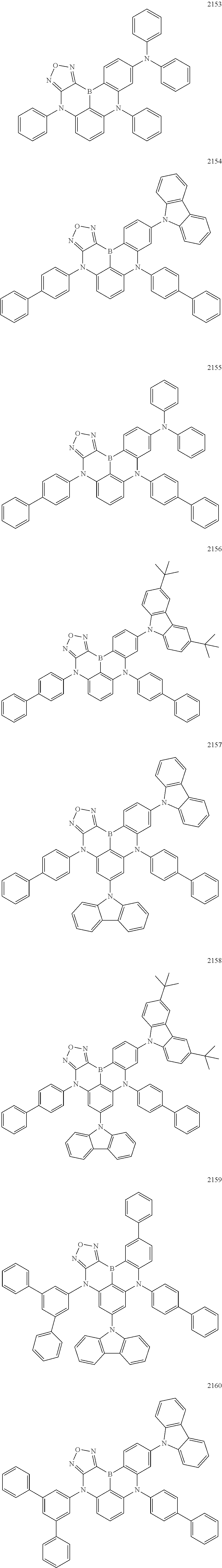

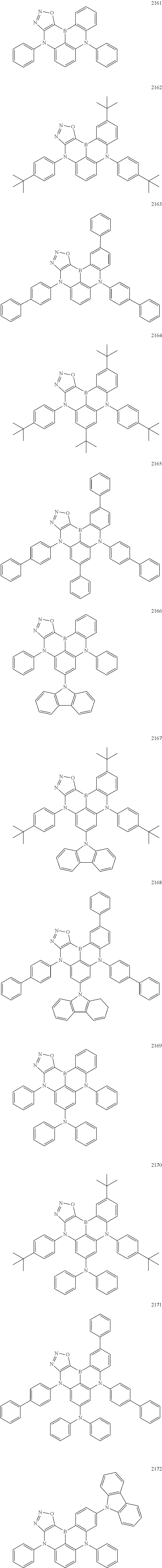

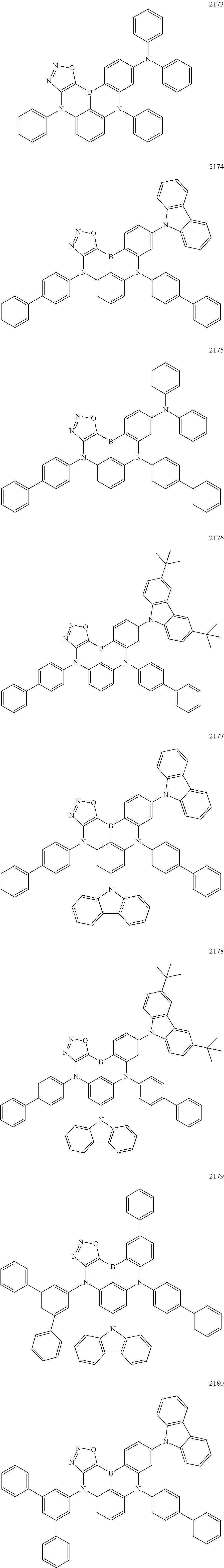

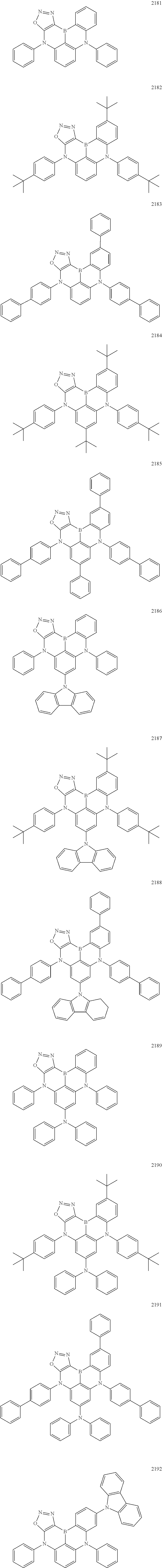

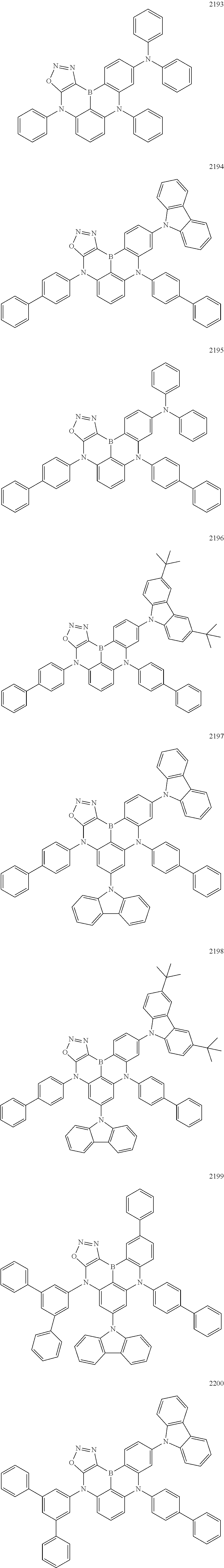

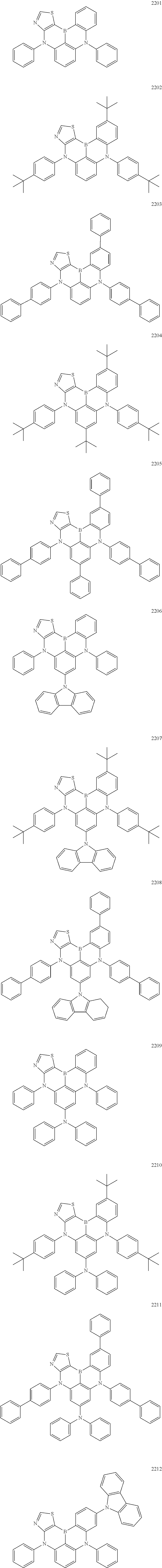

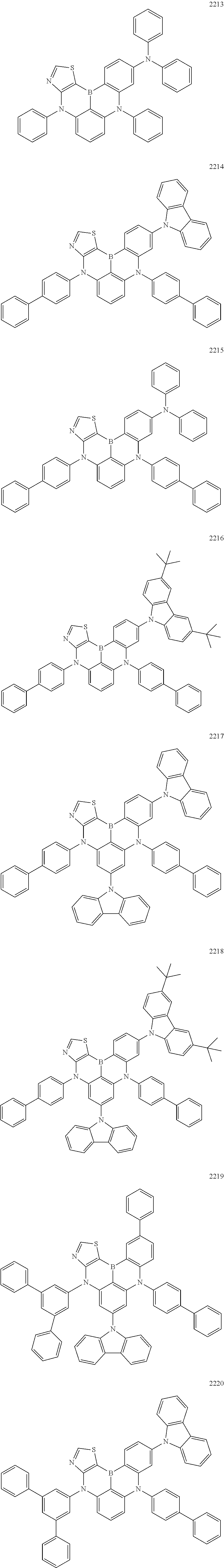

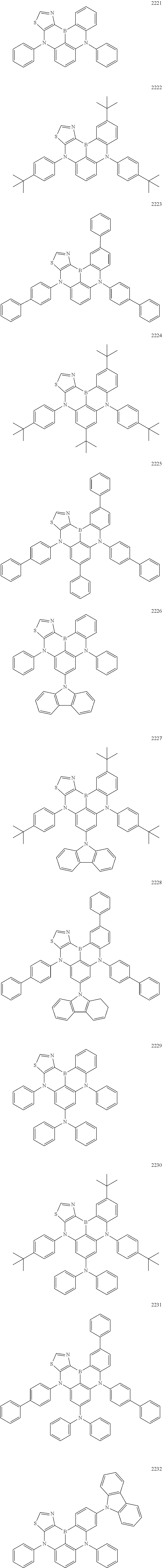

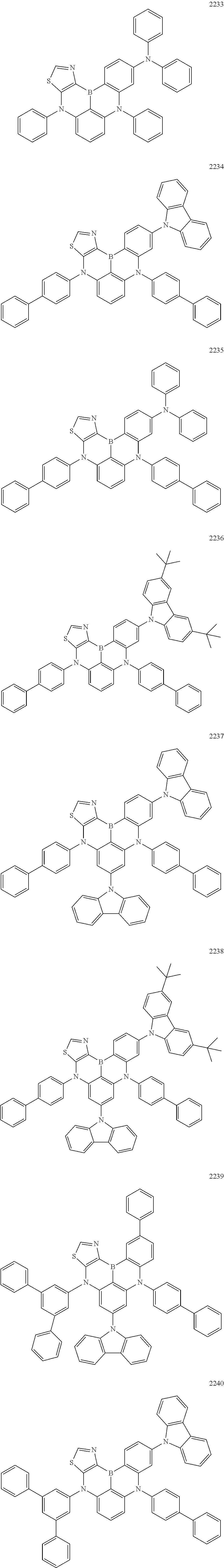

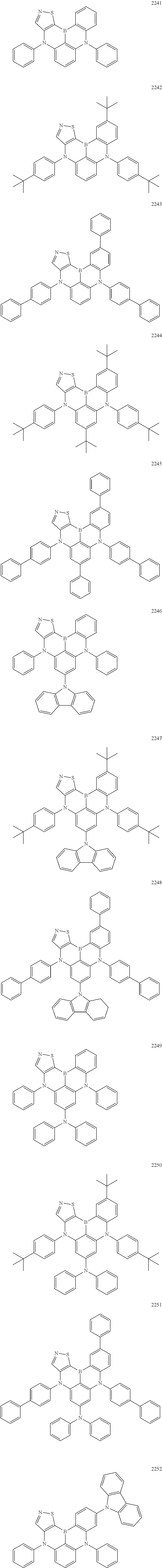

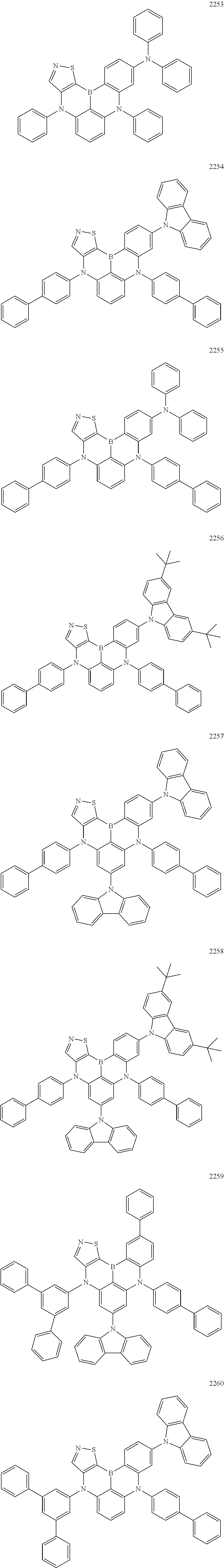

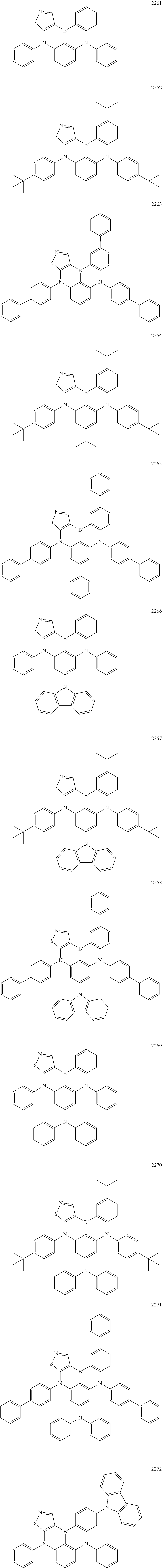

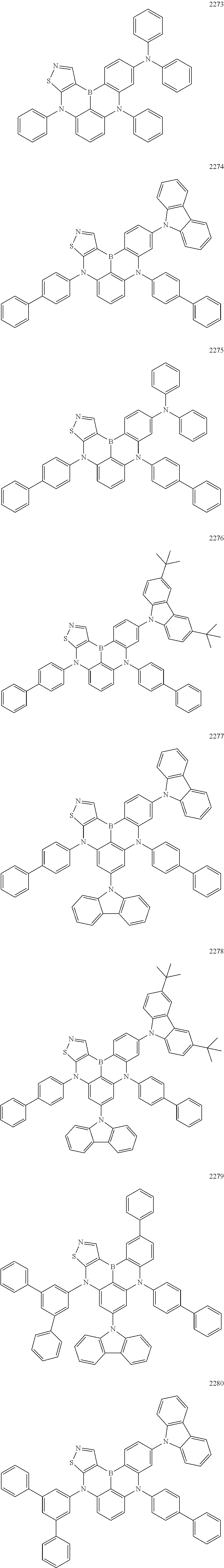

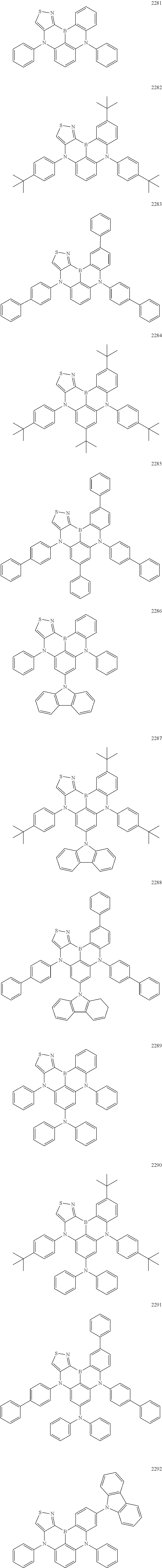

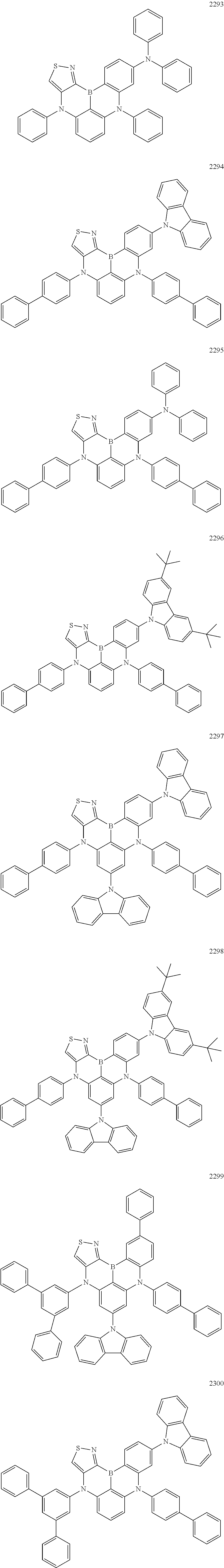

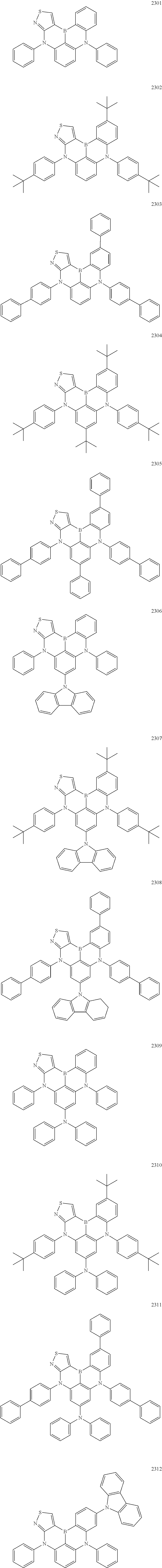

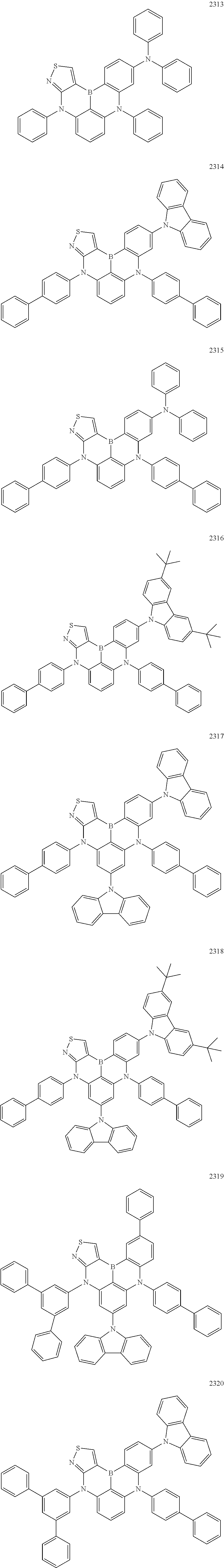

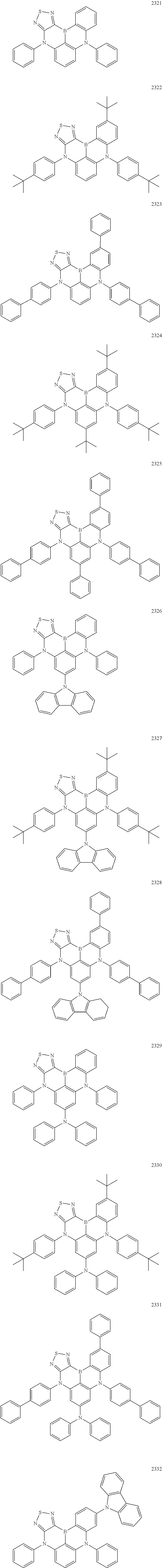

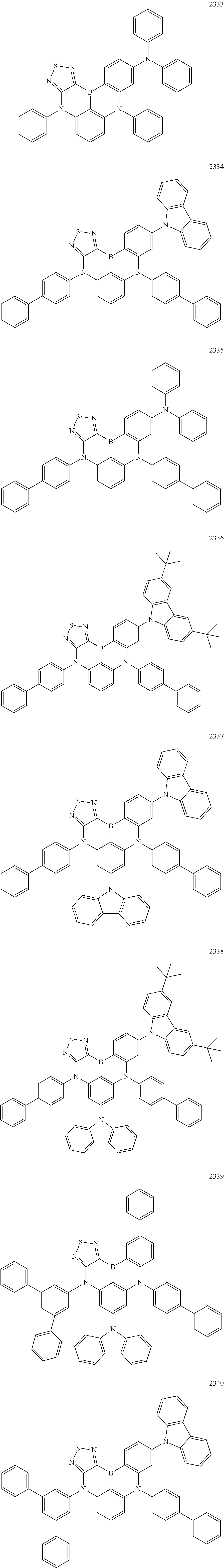

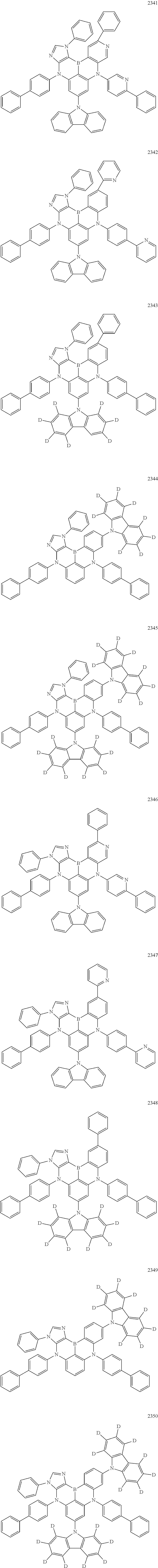

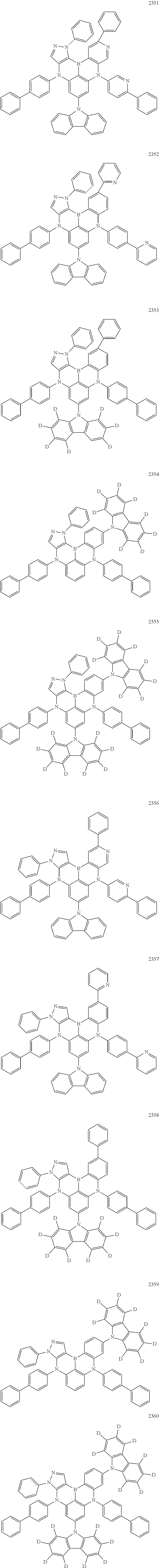

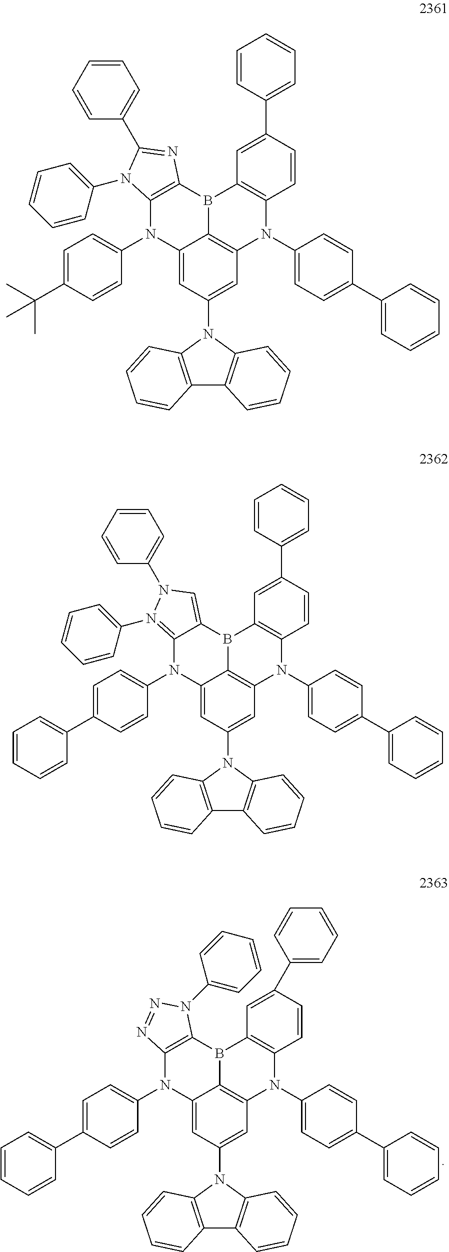

The condensed cyclic compound represented by Formula 1 includes a condensed polycyclic ring structure in which a 5-membered heteroaromatic ring including Z₁ to Z₃ is condensed. The 5-membered heteroaromatic ring includes two or more hetero atoms, so that the singlet (S₁) energy level is increased and thus short-wavelength light can be emitted. In addition, the oscillator strength of the condensed cyclic compound is increased, so that energy transfer is effectively performed in the emission layer, and thus, luminescence efficiency can be increased.

In an embodiment, the condensed cyclic compound represented by Formula 1 may include at least one of the groups represented by Formula 2A or 2B as a substituent. As a result, an increase in the singlet energy level of the condensed cyclic compound is induced, and thus, the emission wavelength may be blue-shifted to a shorter wavelength.

The condensed cyclic compound may emit delayed fluorescence with high efficiency. For example, the condensed cyclic compound may emit thermally activated delayed fluorescence (TADF).

In the condensed cyclic compound, up-conversion of energy from a triplet state to a singlet state may be effectively performed, and thus, high efficiency delayed fluorescence can be emitted.

The triplet energy level and the singlet energy level of the condensed cyclic compound may be evaluated using the structure-optimized DFT method (for example, the DFT method of the Gaussian program) at the B3LYP/6-31 G(d,p) level.

The highest occupied molecular orbital (HOMO), lowest unoccupied molecular orbital (LUMO), and T₁ and S₁ energy levels of some of the condensed cyclic compounds represented by Formula 1 and comparative compounds were measured using the DFT method of the Gaussian program (structure optimization at the B3LYP, 6-31 G(d,p) level). Results thereof are shown in Table 1. S₁ in Table 1 is the energy value calculated when a compound molecule is excited from the ground state to the singlet (S₁) state. T₁ in Table 1 is the energy value calculated when a compound molecule is excited from the ground state to the triplet (T₁) state.

TABLE 1

| Compound | HOMO | LUMO | S₁ | T₁ | Oscillator |
|---|---|---|---|---|---|
| No. | (eV) | (eV) | (eV) | (eV) | strength |
| 37 | −4.864 | −1.368 | 3.078 | 2.582 | 0.718 |
| 948 | −4.866 | −1.339 | 3.075 | 2.564 | 0.484 |
| 956 | −4.995 | −1.457 | 3.088 | 2.572 | 0.614 |
| 1411 | −4.905 | −1.184 | 3.299 | 2.825 | 0.439 |
| 1417 | −5.068 | −1.270 | 3.325 | 2.861 | 0.612 |
| 1868 | −4.979 | −1.258 | 3.328 | 2.818 | 0.483 |
| 1888 | −5.014 | −1.659 | 2.949 | 2.480 | 0.543 |
| 1968 | −4.900 | −1.556 | 2.999 | 2.520 | 0.728 |
| 2008 | −5.011 | −1.484 | 3.091 | 2.562 | 0.471 |
| 2028 | −5.130 | −1.280 | 3.371 | 2.861 | 0.424 |
| 2068 | −5.065 | −1.634 | 2.980 | 2.463 | 0.393 |
| 2088 | −5.024 | −1.639 | 2.932 | 2.430 | 0.503 |
| 2168 | −5.175 | −1.814 | 2.954 | 2.403 | 0.58 |
| 2361 | −4.657 | −1.023 | 3.095 | 2.445 | 0.675 |
| 2362 | −4.923 | −1.421 | 3.072 | 2.623 | 0.561 |
| 2363 | −5.018 | −1.508 | 3.053 | 2.533 | 0.397 |
| CE1 | −5.263 | −2.749 | 2.144 | 1.607 | 0.257 |
| CE2 | −5.436 | −2.602 | 2.537 | 1.788 | 0.216 |

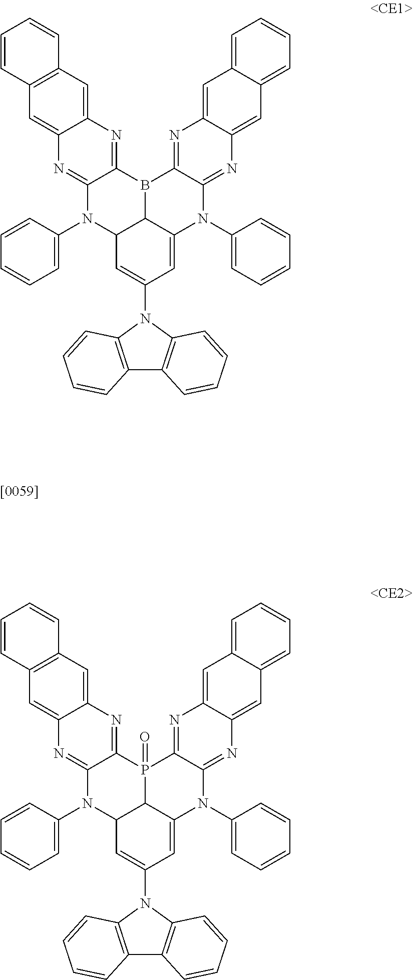 [SMILES: C1=C2C3B(c4nc5cc6ccccc6cc5nc4N2c2ccccc2)c2nc4cc5ccccc5cc4nc2N(c2ccccc2)C3C=C1n1c2ccccc2c2ccccc21 ; O=P12c3nc4cc5ccccc5cc4nc3N(c3ccccc3)C3=CC(n4c5ccccc5c5ccccc54)=CC(C31)N(c1ccccc1)c1nc3cc4ccccc4cc3nc12]

From Table 1, it can be confirmed that the compounds according to the present disclosure have a high S₁ energy level and oscillator strength and exhibit excellent electric characteristics.

The condensed cyclic compound may be suitable for use as an organic layer of an organic light-emitting device, for example, as a material for an emission layer, a hole transport region, and/or an electron transport region in the organic layer.

Accordingly, according to an aspect of another embodiment, an organic light-emitting device may include: a first electrode; a second electrode; and an organic layer between the first electrode and the second electrode, the organic layer including an emission layer and at least one of the condensed cyclic compounds described above.

Since the organic light-emitting device has an organic layer including the condensed cyclic compound, the organic light-emitting device may have a low driving voltage, high efficiency, high luminance, high quantum efficiency, and a long lifespan.

In an embodiment, in the organic light-emitting device,

the first electrode may be an anode and the second electrode may be a cathode,

the organic layer includes a hole transport region between the first electrode and the emission layer and an electron transport region between the emission layer and the second electrode,

the hole transport region includes a hole injection layer, a hole transport layer, an electron-blocking layer, or any combination thereof, and

the electron transport region may include a hole-blocking layer, an electron transport layer, an electron injection layer, or any combination thereof, and embodiments of the present disclosure are not limited thereto.

For example, the condensed cyclic compound may be included in an emission layer of the organic light-emitting device.

In an embodiment, an emission layer of the organic light-emitting device may include a host and a dopant, and the dopant may include at least one of the condensed cyclic compounds.

For example, the emission layer may emit delayed fluorescence. The delayed fluorescence may be fluorescence emitted from the condensed cyclic compound as a dopant.

The emission layer may emit red light, green light, or blue light. For example, the emission layer may emit blue light. The maximum emission wavelength of the blue light may be about 420 nm to about 500 nm, for example, about 430 nm to about 490 nm, about 440 nm to about 480 nm, or about 450 nm to about 470 nm.

In an embodiment, the condensed cyclic compound may be included in at least one of the hole transport region, the electron transport region, or a combination thereof.

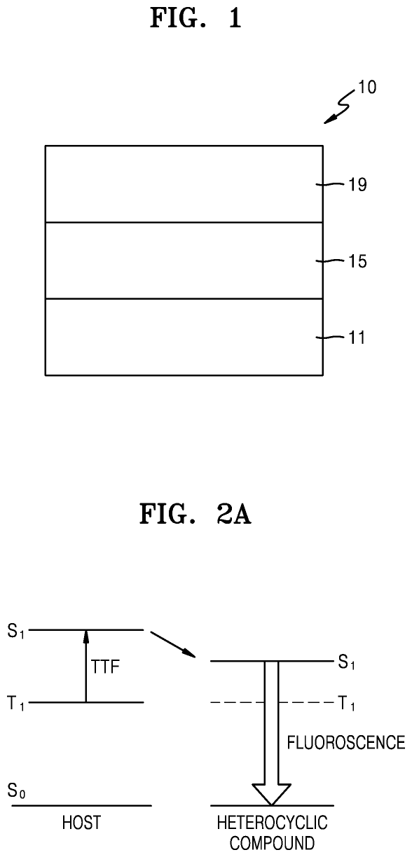

### Description of FIG. 1

FIG. 1 is a schematic cross-sectional view of an organic light-emitting device 10 according to an embodiment. Hereinafter, the structure and manufacturing method of the organic light-emitting device 10 according to an exemplary embodiment of the present disclosure will be described in connection with FIG. 1.

The organic light-emitting device 10 of FIG. 1 may include a first electrode 11, an organic layer 15, and a second electrode 19, which may be sequentially layered in this stated order.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

The first electrode 11 may be produced by depositing or sputtering, onto the substrate, a material for forming the first electrode 11. The first electrode 11 may be an anode. The material for forming the first electrode 11 may be materials with a high work function for easy hole injection.

### First Electrode 11

The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). In one or more embodiments, the material for forming the first electrode 11 may be metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including a plurality of layers. For example, the first electrode 11 may have a three-layered structure of ITO/Ag/ITO, but the structure of the first electrode 11 is not limited thereto.

### Organic Layer 15

The organic layer 15 is located on the first electrode 11.

The organic layer 15 may include a hole transport region, an emission layer, and an electron transport region.

### Hole Transport Region in Organic Layer 15

The hole transport region may be between the first electrode 11 and the emission layer.

The hole transport region may include at least one a hole injection layer, a hole transport layer, an electron-blocking layer, a buffer layer, or a combination thereof.

The hole transport region may include only either a hole injection layer or a hole transport layer. In one or more embodiments, the hole transport region may have a hole injection layer/hole transport layer structure or a hole injection layer/hole transport layer/electron-blocking layer structure, wherein, for each structure, respective layers are sequentially stacked in this stated order from the first electrode 11.

When the hole transport region includes a hole injection layer, the hole injection layer may be formed on the first electrode 11 by using one or more suitable methods, such as vacuum deposition, spin coating, casting, and Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10-3 torr, and a deposition rate of about 0.01 Å/sec to about 100 Å/sec. However, the deposition conditions are not limited thereto.

When a hole injection layer is formed by spin coating, the spin coating may be performed at a rate in a range of about 2,000 revolutions per minute (rpm) to about 5,000 rpm and at a temperature in a range of about 80° C. to 200° C. to facilitate removal of a solvent after the spin coating, though the conditions may vary depending on a compound used as a hole injection material and a structure and thermal properties of a desired hole injection layer, but embodiments are not limited thereto.

The conditions for forming the hole transport layer and the electron-blocking layer may be the same as the conditions for forming the hole injection layer.

The hole transport region may include at least one of m-MTDATA, TDATA, 2-TNATA, NPB, β-NPB, TPD, spiro-TPD, spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), (polyaniline)/poly(4-styrenesulfonate) (PANI/PSS), a compound represented by Formula 201, a compound represented by Formula 202, or a combination thereof:

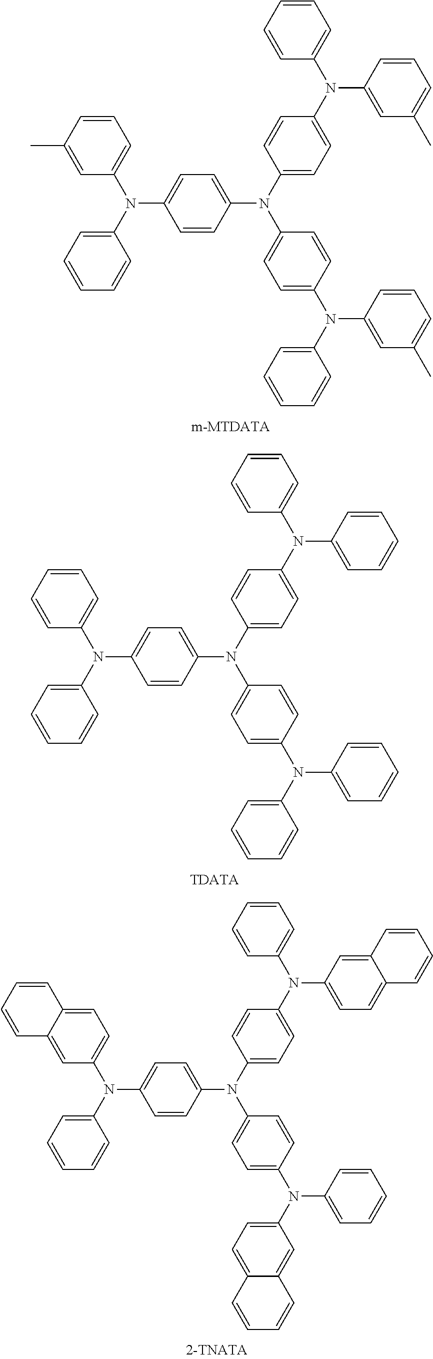

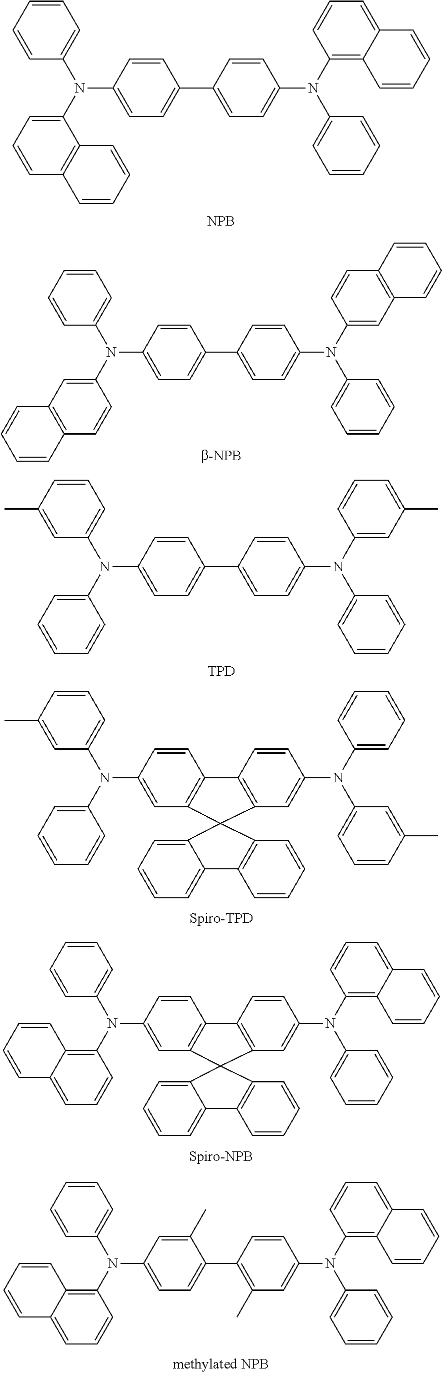

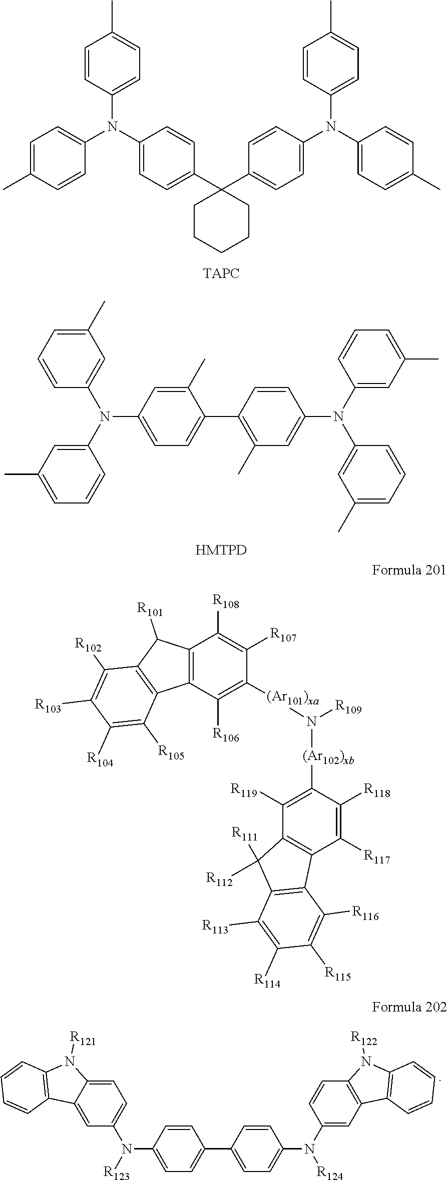

Ar₁₀₁ and Ar₁₀₂ in Formula 201 may each independently be:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, or a pentacenylene group; or

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, or a pentacenylene group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or a combination thereof.

xa and xb in Formula 201 may each independently be an integer from 0 to 5, or 0, 1, or 2. For example, xa may be 1 and xb may be 0, but xa and xb are not limited thereto.

R₁₀₁ to R₁₀₈, R₁₁₁ to R₁₁₉ and R₁₂₁ to R₁₂₄ in Formulae 201 and 202 may each independently be:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, etc.), or a C₁-C₁₀ alkoxy group (for example, a methoxy group, an ethoxy group, a propoxy group, a butoxy group, a pentoxy group, etc.);

a C₁-C₁₀ alkyl group or a C₁-C₁₀ alkoxy group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, or any combination thereof;

a phenyl group, a naphthyl group, an anthracenyl group, a fluorenyl group, or a pyrenyl group; or

a phenyl group, a naphthyl group, an anthracenyl group, a fluorenyl group, or a pyrenyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, or any combination thereof,

but embodiments of the present disclosure are not limited thereto.

R₁₀₉ in Formula 201 may be:

a phenyl group, a naphthyl group, an anthracenyl group, or a pyridinyl group; or

a phenyl group, a naphthyl group, an anthracenyl group, or a pyridinyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, a pyridinyl group, or any combination thereof.

According to an embodiment, the compound represented by Formula 201 may be represented by Formula 201A below, but embodiments of the present disclosure are not limited thereto:

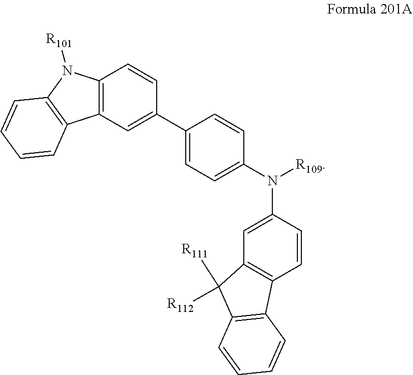

R₁₀₁, R₁₁₁, R₁₁₂, and R₁₀₉ in Formula 201A are each the same as described in the present specification.

For example, the compound represented by Formula 201, and the compound represented by Formula 202 may each include compounds HT1 to HT20 illustrated below, but are not limited thereto:

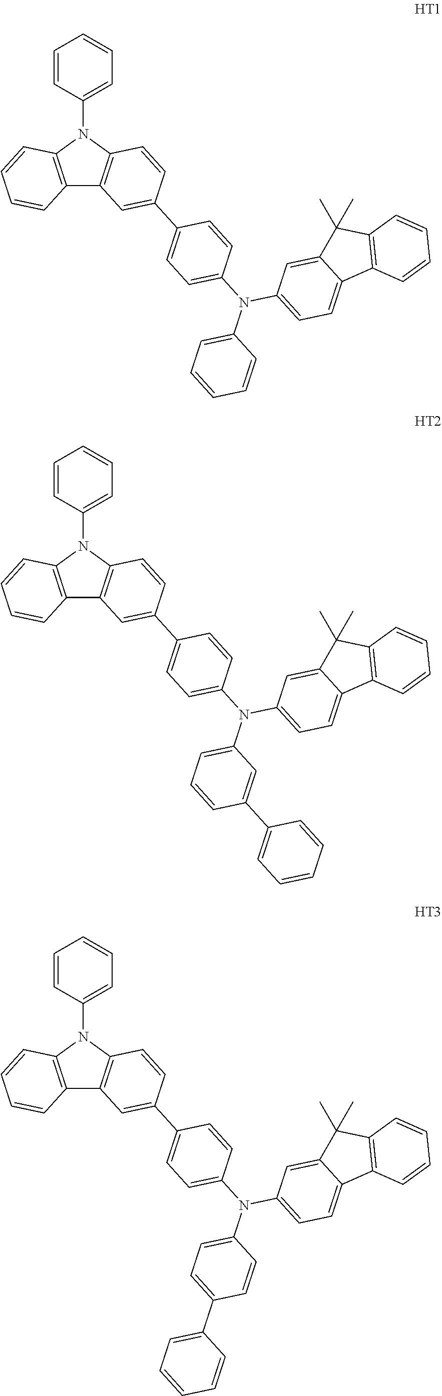 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

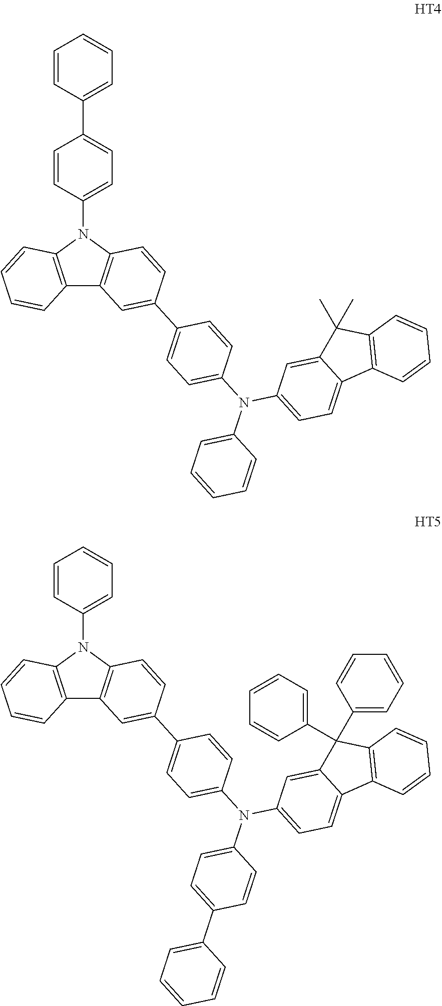 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

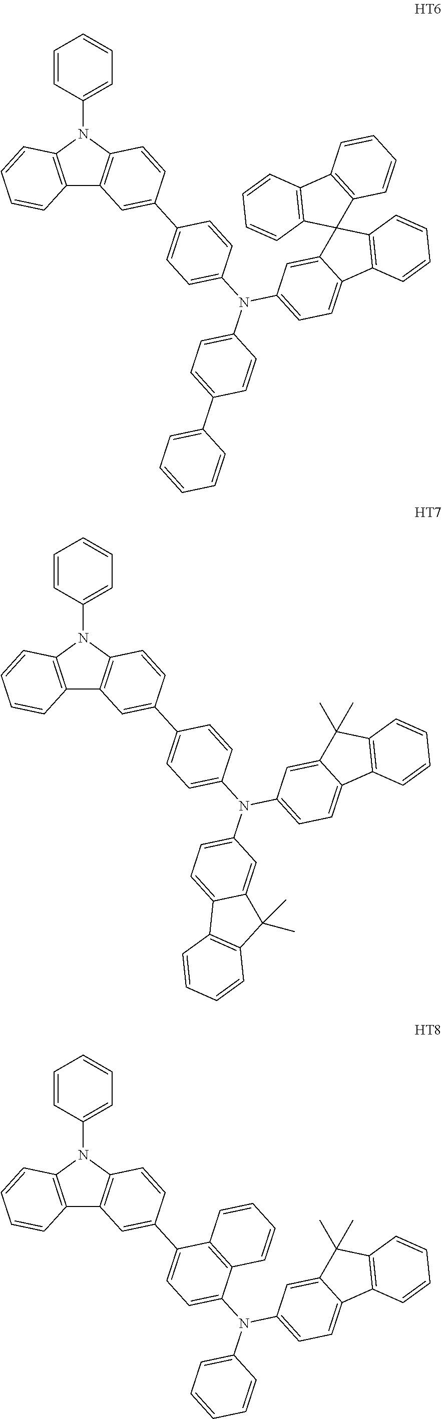 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

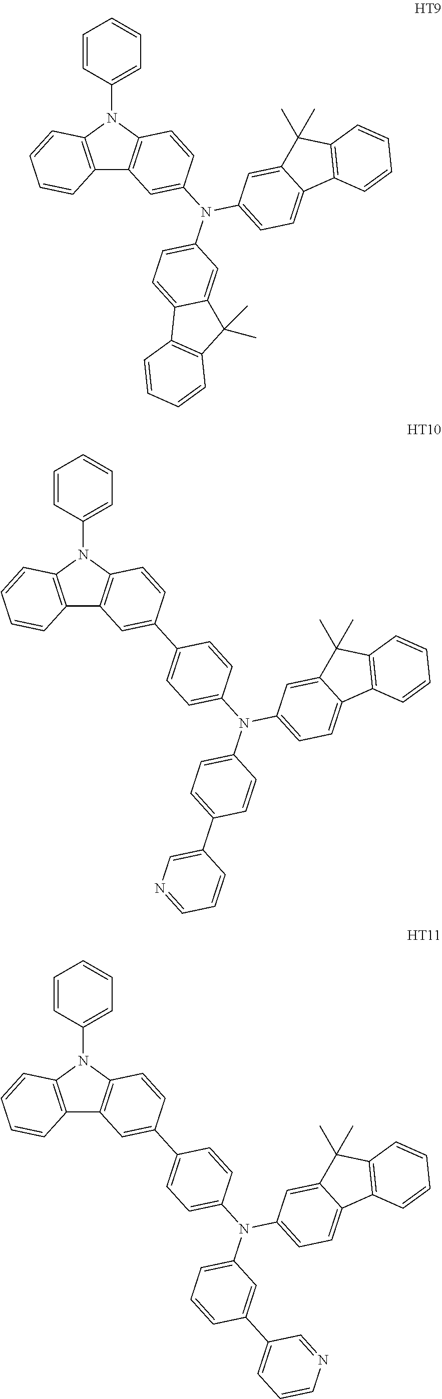 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

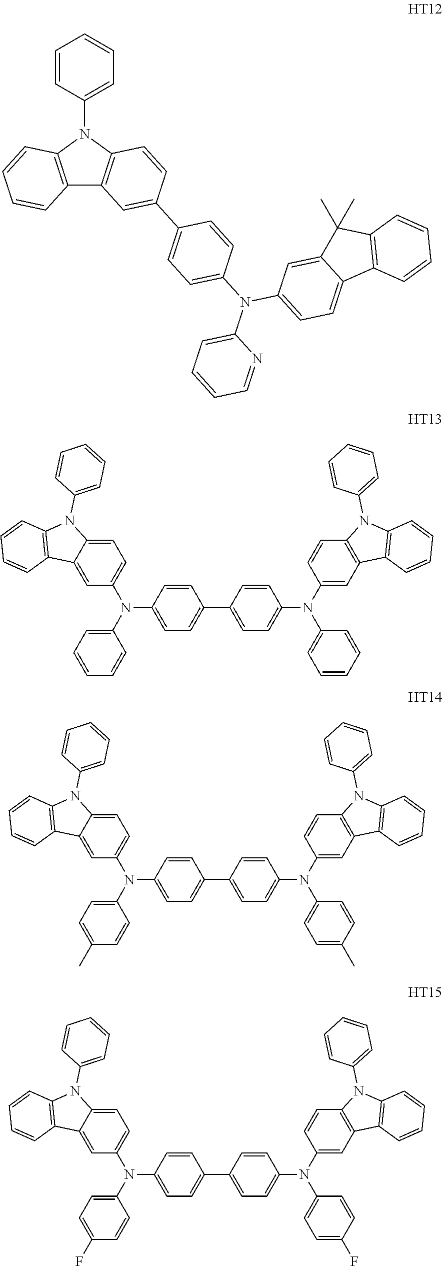 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

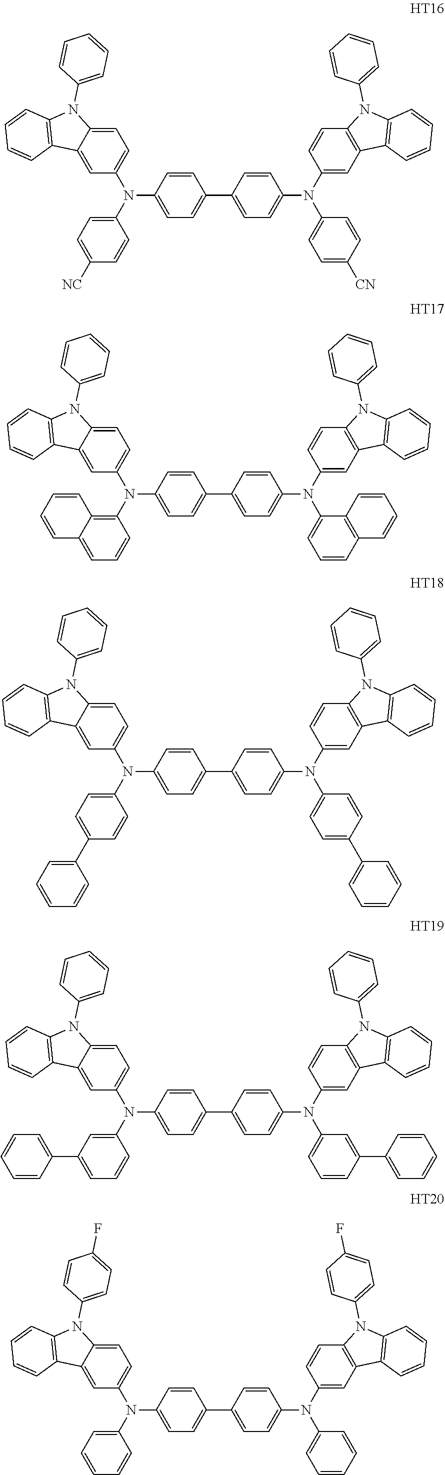

A thickness of the hole transport region may be in the range of about 100 Å to about 10,000 Å, for example, about 100 Å to about 1,000 Å. When the hole transport region includes at least one of a hole injection layer and a hole transport layer, a thickness of the hole injection layer may be in a range of about 100 Å to about 10,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one of a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; and a cyano group-containing compound, such as Compound HT-D1 or Compound HT-D2 below, but are not limited thereto.

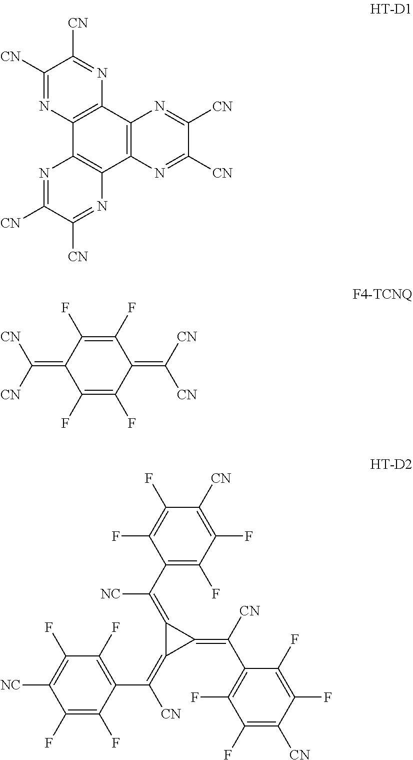

The hole transport region may include a buffer layer.

Also, the buffer layer may compensate for an optical resonance distance according to a wavelength of light emitted from the emission layer, and thus, efficiency of a formed organic light-emitting device may be improved.

The emission layer may be formed on the hole transport region by using one or more suitable methods, such as vacuum deposition, spin coating, casting, or LB deposition. When the emission layer is formed by vacuum deposition or spin coating, the deposition or coating conditions may be similar to those applied in forming the hole injection layer although the deposition or coating conditions may vary according to a material that is used to form the hole transport layer.

The hole transport region may further include an electron-blocking layer. The electron-blocking layer may include a material available in the art, for example, mCP, but embodiments of the present disclosure are not limited thereto.

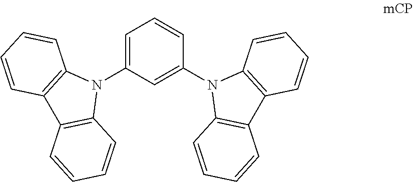 [SMILES: c1cc(-n2c3ccccc3c3ccccc32)cc(-n2c3ccccc3c3ccccc32)c1]

A thickness of the electron-blocking layer may be about 50 Å to about 1,000 Å, for example about 70 Å to about 500 Å. When the thickness of the electron-blocking layer is within the range described above, the electron-blocking layer may have satisfactory electron blocking characteristics without a substantial increase in driving voltage.

### Emission Layer in Organic Layer 15

When the organic light-emitting device 10 is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The emission layer may include the condensed cyclic compound represented by Formula 1.

In an embodiment, the emission layer may include the condensed cyclic compound represented by Formula 1 alone.

In an embodiment, the emission layer includes a host and a dopant, and the dopant may include the condensed cyclic compound represented by Formula 1.

When the emission layer includes a host and a dopant, the amount of the dopant may be in the range of about 0.01 part by weight to about 20 parts by weight based on 100 parts by weight of the emission layer. However, the amount of the dopant included in the emission layer is not limited thereto. When the amount of the dopant satisfies the above range, it may be possible to realize emission without extinction phenomenon.

A thickness of the emission layer may be in a range of about 100 Å to about 1,000 Å, for example, about 200 Å to about 600 Å. When the thickness of the emission layer is within these ranges, excellent light-emission characteristics may be obtained without a substantial increase in driving voltage.

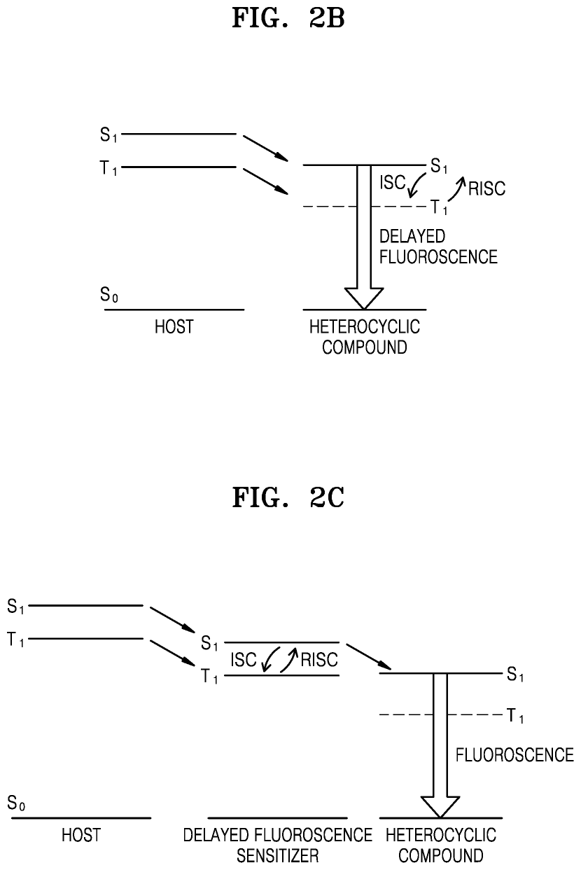

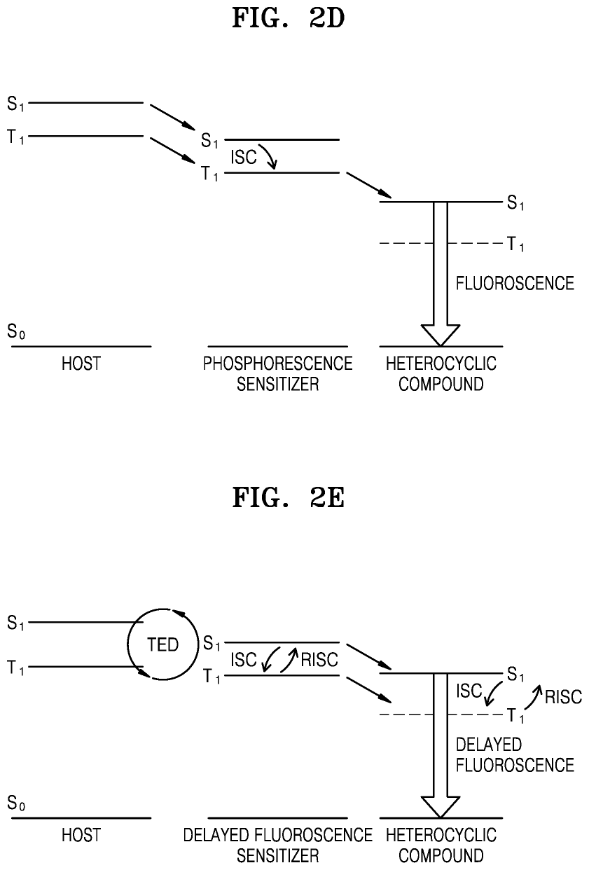

### FIRST EMBODIMENT—DESCRIPTION OF FIG. 2A

In the First Embodiment, the condensed cyclic compound may be a fluorescence emitter. According to the First Embodiment, the emission layer may further include a host (hereinafter, referred to as ‘Host A’, and Host A may not be identical to the condensed cyclic compound). Host A may be understood by referring to the description of the host material provided herein, but embodiments are not limited thereto. Host A may be a fluorescent host.

The general energy transfer of the First Embodiment will be described according to FIG. 2A.

Singlet excitons may be produced from Host A in the emission layer, and singlet excitons produced from Host A may be transferred to a fluorescence emitter through Förster energy transfer (FRET).

A ratio of singlet excitons produced from Host A may be 25%, and thus, 75% of triplet excitons produced from Host A may be fused to one another to be converted into singlet excitons. Thus, the efficiency of the organic light-emitting device may be further improved. That is, the efficiency of an organic light-emitting device may be further improved by using a triplet-triplet fusion mechanism.

According to the First Embodiment, a ratio of emission components emitted from the condensed cyclic compound to the total emission components emitted from the emission layer may be 80% or greater, for example, 90% or greater. In some embodiments, a ratio of emission components emitted from the condensed cyclic compound may be 95% or greater to the total emission components emitted from the emission layer.

The condensed cyclic compound may emit fluorescence, and the host may not emit light.

In the First Embodiment, when the emission layer further includes Host A, in addition to the condensed cyclic compound, an amount of the condensed cyclic compound may be about 50 parts by weight or less, e.g., about 30 parts by weight or less, based on 100 parts by weight of the emission layer, and an amount of Host A in the emission layer may be about 50 parts by weight or greater, e.g., about 70 parts by weight or greater, based on 100 parts by weight of the emission layer, but embodiments are not limited thereto.

In the First Embodiment, when the emission layer further includes Host A, in addition to the condensed cyclic compound, Host A and the condensed cyclic compound may satisfy Condition A:

E(H_{A})_{S1}>E_{S1} Condition A

wherein, in Condition A,

E(H_{A})_{S1} indicates a lowest excited singlet energy level of Host A, and

E_{S1} indicates a lowest excited singlet energy level of the condensed cyclic compound.

Here, E(H_{A})_{S1} and E_{S1} may be evaluated using the density functional theory (DFT) method of the Gaussian program, which is structure-optimized at the B3LYP/6-31 G(d,p) level.

### SECOND EMBODIMENT—DESCRIPTIONS OF FIG. 2B

In the Second Embodiment, the condensed cyclic compound may be a delayed fluorescence emitter. According to the Second Embodiment, the emission layer may further include a host (hereinafter, referred to as ‘Host B’, and Host B may not be identical to the condensed cyclic compound). Host B may be understood by referring to the description of the host material provided herein, but embodiments are not limited thereto.

The general energy transfer of the Second Embodiment will be described according to FIG. 2B.

25% of singlet excitons produced from Host B in the emission layer may be transferred to a delayed fluorescence emitter through FRET. In addition, 75% of triplet excitons produced from Host B in the emission layer may be transferred to a delayed fluorescence emitter through Dexter energy transfer. Energy transferred to a triplet state of a delayed fluorescence emitter may undergo reverse intersystem crossing to a singlet state. Accordingly, by transferring all the singlet excitons and triplet excitons generated in the emission layer to the condensed cyclic compound, an organic light-emitting device having improved efficiency can be obtained.

According to the Second Embodiment, a ratio of emission components emitted from the condensed cyclic compound to the total emission components emitted from the emission layer may be 80% or greater, for example, 90% or greater. In some embodiments, a ratio of emission components emitted from the condensed cyclic compound may be 95% or greater to the total emission components emitted from the emission layer.

Here, the condensed cyclic compound may emit fluorescence and/or delayed fluorescence, and the emission components of the condensed cyclic compound may be a total of prompt emission components of the condensed cyclic compound and delayed fluorescence components by reverse intersystem crossing of the condensed cyclic compound. In addition, Host B may not emit light.

In the Second Embodiment, when the emission layer further includes Host B, in addition to the condensed cyclic compound, an amount of the condensed cyclic compound may be about 50 parts by weight or less, e.g., about 30 parts by weight or less, based on 100 parts by weight of the emission layer, and an amount of Host B in the emission layer may be about 50 parts by weight or greater, e.g., about 70 parts by weight or greater, based on 100 parts by weight of the emission layer, but embodiments are not limited thereto.

In the Second Embodiment, when the emission layer further includes Host B, in addition to the condensed cyclic compound, Host B and the condensed cyclic compound may satisfy Condition B:

E(H_{B})_{S1}>E_{S1} Condition B

wherein, in Condition B,

E(H_{B})_{S1} indicates a lowest excited singlet energy level of Host B, and

E_{S1} indicates a lowest excited singlet energy level of the condensed cyclic compound.

Here, E(H_{B})_{S1} and E_{S1} may be evaluated using the DFT method of the Gaussian program, which is structure-optimized at the B3LYP/6-31 G(d,p) level.

### THIRD EMBODIMENT—DESCRIPTIONS OF FIG. 2C

In the Third Embodiment, the condensed cyclic compound may be used as a fluorescence emitter, and the emission layer may include a sensitizer, e.g., a delayed fluorescence sensitizer. In the Third Embodiment, the emission layer may further include a host (hereinafter, the host may be referred to as ‘Host C’, and Host C may not be identical to the condensed cyclic compound and the sensitizer) and a sensitizer (hereinafter, the sensitizer may be referred to as ‘Sensitizer A’, and Sensitizer A may not be identical to Host C and the condensed cyclic compound). Host C and Sensitizer A may respectively be understood by referring to the description of the host material and the sensitizer material provided herein, but embodiments are not limited thereto.

In the Third Embodiment, a ratio of emission components of the condensed cyclic compound may be about 80% or greater, for example, 90% or greater (or for example, 95% or greater) to the total emission components emitted from the emission layer. For example, the condensed cyclic compound may emit fluorescence. In addition, each of Host C and Sensitizer A may not emit light.

The general energy transfer of the Third Embodiment will be described according to FIG. 2C.

Singlet and triplet excitons may be produced from Host C in the emission layer, and singlet and triplet excitons produced from Host C may be transferred to Sensitizer A and then to the condensed cyclic compound through FRET. 25% of singlet excitons produced from Host C may be transferred to Sensitizer A through FRET, and energy of 75% of triplet excitons produced from Host C may be transferred to singlet and triplet states of Sensitizer A. Energy transferred to a triplet state of Sensitizer A may undergo reverse intersystem crossing to a singlet state, and then, singlet energy of Sensitizer A may be transferred to the condensed cyclic compound through FRET.

Accordingly, by transferring all the singlet excitons and triplet excitons generated in the emission layer to the dopant, an organic light-emitting device having improved efficiency can be obtained. In addition, since an organic light-emitting device can be obtained with significantly reduced energy loss, the lifespan characteristics of the organic light-emitting device can be improved.

In the Third Embodiment, when the emission layer further includes Host C and Sensitizer A, in addition to the condensed cyclic compound, Host C and Sensitizer A may satisfy Condition C-1 and/or C-2:

S₁(H_{C})≥S₁(S_{A}) Condition C-1

S₁(S_{A})≥S₁(H_{C}) Condition C-2

wherein, in Conditions C-1 and C-2,
S₁(H_{C}) indicates a lowest excited singlet energy level of Host C,
S₁(S_{A}) indicates a lowest excited singlet energy level of Sensitizer A, and

S₁(HC) indicates a lowest excited singlet energy level of the condensed cyclic compound.

S₁(H_{C}), S₁(S_{A}), and S₁(HC) may be evaluated using the DFT method of the Gaussian program, which is structure-optimized at the B3LYP/6-31 G(d,p) level.

When Host C, Sensitizer A, and the condensed cyclic compound satisfy Condition C-1 and/or C-2, FRET from Sensitizer A to the condensed cyclic compound may be facilitated, and accordingly, the organic light-emitting device may have improved luminescence efficiency.

### FOURTH EMBODIMENT—DESCRIPTION OF FIG. 2D

In the Fourth Embodiment, the condensed cyclic compound may be used as a fluorescence emitter, and the emission layer may include a sensitizer, e.g., a phosphorescence sensitizer. In the Fourth Embodiment, the emission layer may further include a host (hereinafter, the host may be referred to as ‘Host D’, and Host D may not be identical to the condensed cyclic compound and the sensitizer) and a sensitizer (hereinafter, the sensitizer may be referred to as ‘Sensitizer B’, and Sensitizer B may not be identical to Host D and the condensed cyclic compound). Host D and Sensitizer B may respectively be understood by referring to the description of the host material and the sensitizer material provided herein, but embodiments are not limited thereto.

In the Fourth Embodiment, a ratio of emission components of the condensed cyclic compound may be about 80% or greater, for example, 90% or greater (or for example, 95% or greater) to the total emission components emitted from the emission layer. For example, the condensed cyclic compound may emit fluorescence. In addition, Host D and Sensitizer B may not each emit light.

The general energy transfer of the Fourth Embodiment will be described according to FIG. 2D.

75% of triplet excitons produced from Host D in the emission layer may be transferred to Sensitizer B through Dexter energy transfer, and energy of 25% of singlet excitons produced from Host D may be transferred to singlet and triplet states of Sensitizer B. Energy transferred to a singlet state of Sensitizer B may undergo ISC to a triplet state, and then, triplet energy of Sensitizer B may be transferred to the condensed cyclic compound through FRET.

Accordingly, by transferring all the singlet excitons and triplet excitons generated in the emission layer to the dopant, an organic light-emitting device having improved efficiency can be obtained. In addition, since an organic light-emitting device can be obtained with significantly reduced energy loss, the lifespan characteristics of the organic light-emitting device can be improved.

In the Fourth Embodiment, when the emission layer further includes Host D and Sensitizer B, in addition to the condensed cyclic compound, Host D and Sensitizer B may satisfy Condition D-1 and/or D-2:

T₁(H_{D})≥T₁(S_{B}) Condition D-1

T₁(S_{B})≥S₁(H_{C}) Condition D-2

wherein, in Conditions D-1 and D-2,

T₁(H_{D}) indicates a lowest excited triplet energy level of Host D,

T₁(S_{B}) indicates a lowest excited triplet energy level of Sensitizer B, and

S₁(HC) indicates a lowest excited singlet energy level of the condensed cyclic compound.

T₁(H_{D}), T₁(S_{B}), and S₁(HC) may be evaluated using the DFT method of the Gaussian program, which is structure-optimized at the B3LYP/6-31 G(d,p) level.

When Host D, Sensitizer B, and the condensed cyclic compound satisfy Condition D-1 and/or D-2, FRET from Sensitizer B to the condensed cyclic compound may be facilitated, and accordingly, the organic light-emitting device may have improved luminescence efficiency.

In the Third Embodiment and the Fourth Embodiment, an amount of the sensitizer in the emission layer may be in a range of about 5 percent by weight (wt %) to about 50 wt %, or for example, about 10 wt % to about 30 wt %. When the amount is within this range, energy transfer in the emission layer may effectively occur. Thus, the organic light-emitting device may have high efficiency and long lifespan.

In the Third Embodiment and the Fourth Embodiment, an amount of the condensed cyclic compound in the emission layer may be in a range of about 0.01 wt % to about 15 wt %, or for example, about 0.05 wt % to about 3 wt %, but embodiments are not limited thereto.

In the Third Embodiment and the Fourth Embodiment, the sensitizer and the condensed cyclic compound may further satisfy Condition 5:

0 μs<T_{decay}(HC)<5 μs Condition 5

wherein, in Condition 5,

T_{decay}(HC) indicates a decay time of the condensed cyclic compound.

The decay time of the condensed cyclic compound was measured from a time-resolved photoluminescence (TRPL) spectrum at room temperature of a film (hereinafter, referred to as “Film (HC)”) having a thickness of 40 nm formed by vacuum-depositing the host and the condensed cyclic compound included in the emission layer on a quartz substrate at a weight ratio of 90:10 at a vacuum degree of 10-7 torr.

### FIFTH EMBODIMENT—DESCRIPTION OF FIG. 2E

In the Fifth Embodiment, the condensed cyclic compound may be used as a delayed fluorescence emitter, and the emission layer may include a sensitizer, e.g., a delayed fluorescence sensitizer. In the Fifth Embodiment, the emission layer may further include a host (hereinafter, the host may be referred to as ‘Host E’, and Host E may not be identical to the condensed cyclic compound and the sensitizer) and a sensitizer (hereinafter, the sensitizer may be referred to as ‘Sensitizer C’, and Sensitizer C may not be identical to Host E and the condensed cyclic compound). Host E and Sensitizer C may respectively be understood by referring to the description of the host material and the sensitizer material provided herein, but embodiments are not limited thereto.

In the Fifth Embodiment, a ratio of emission components of the condensed cyclic compound may be about 80% or greater, for example, about 90% or greater (or for example, 95% or greater) to the total emission components emitted from the emission layer. In some embodiments, the condensed cyclic compound may emit fluorescence and/or delayed fluorescence. In addition, Host E and Sensitizer C may not each emit light.

Here, the condensed cyclic compound may emit fluorescence and/or delayed fluorescence, and the emission components of the condensed cyclic compound may be a total of prompt emission components of the condensed cyclic compound and delayed fluorescence components by reverse intersystem crossing of the condensed cyclic compound.

The general energy transfer of the Fifth Embodiment will be described according to FIG. 2E.

25% of singlet excitons produced from Host E in the emission layer may be transferred to a singlet state of Sensitizer C through FRET, and energy of 75% of triplet excitons produced from Host E may be transferred to a triplet state of Sensitizer C, and then singlet energy of Sensitizer C may be transferred to the condensed cyclic compound through FRET. Subsequently, the triplet energy of Sensitizer C may be transferred to the condensed cyclic compound through Dexter energy transfer. Energy transferred to a triplet state of Sensitizer C may undergo reverse intersystem crossing to a singlet state. Further, in a case of Sensitizer C, energy of triplet excitons produced from Sensitizer C may undergo reverse transfer to Host E and then to the condensed cyclic compound, thus emitting by reverse intersystem transfer.

Accordingly, by transferring all the singlet excitons and triplet excitons generated in the emission layer to the dopant, an organic light-emitting device having improved efficiency can be obtained. In addition, since an organic light-emitting device can be obtained with significantly reduced energy loss, the lifespan characteristics of the organic light-emitting device can be improved.

In the Fifth Embodiment, when the emission layer further includes Host E and Sensitizer C, in addition to the condensed cyclic compound, Host E and Sensitizer C may satisfy Condition E-1, E-2, E-3, or a combination thereof:

S₁(H_{E})≥S₁(S_{C}) Condition E-1

S₁(S_{C})≥S₁(HC) Condition E-2

T₁(S_{C})≥T₁(HC) Condition E-3

wherein, in Conditions E-1, E-2, and E-3,

S₁(H_{E}) indicates a lowest excited singlet energy level of Host E,

S₁(S_{C}) indicates a lowest excited singlet energy level of Sensitizer C,

S₁(HC) indicates a lowest excited singlet energy level of the condensed cyclic compound,

T₁(S_{C}) indicates a lowest excited triplet energy level of Sensitizer C, and

T₁(HC) indicates a lowest excited triplet energy level of the condensed cyclic compound.

S₁(H_{E}), S₁(S_{C}), S₁(HC), T₁(S_{C}), and T₁(HC) may be evaluated using the DFT method of the Gaussian program, which is structure-optimized at the B3LYP/6-31 G(d,p) level.

When Host E, Sensitizer C, and the condensed cyclic compound satisfy Condition E-1, E-2, and/or E-3, Dexter transfer FRET from Sensitizer C to the condensed cyclic compound may be facilitated, and accordingly, the organic light-emitting device may have improved luminescence efficiency.

In the Fifth Embodiment, an amount of Sensitizer C in the emission layer may be in a range of about 5 wt % to about 50 wt %, or for example, about 10 wt % to about 30 wt %. When the amount is within this range, energy transfer in the emission layer may be effectively occurred. Thus, the organic light-emitting device may have high efficiency and long lifespan.

In the Fifth Embodiment, an amount of the condensed cyclic compound in the emission layer may be in a range of about 0.01 wt % to about 15 wt %, or for example, about 0.05 wt % to about 3 wt %, but embodiments are not limited thereto.

### Host

The host may not include a metal atom.

In one or more embodiments, the host may include one kind of host. When the host includes one host, the one host may be a bipolar host, an electron-transporting host, a hole-transporting host, or a combination thereof, which will be described later.

In one or more embodiments, the host may include a mixture of two or more different hosts. For example, the host may be a mixture of an electron-transporting host and a hole-transporting host, a mixture of two types of electron-transporting hosts different from each other, or a mixture of two types of hole-transporting hosts different from each other. The electron-transporting host and the hole-transporting host may be understood by referring to the related description to be presented later.

In one or more embodiments, the host may include an electron-transporting host including at least one electron-transporting moiety and a hole-transporting host that is free of an electron-transporting moiety.

The electron-transporting moiety used herein may be a cyano group, a π electron-deficient nitrogen-containing cyclic group, a group represented by one of the following chemical structures, or a combination thereof:

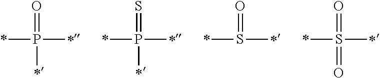

wherein, in Formulae ET-moiety, *, *′, and *″ may each indicate a binding site to an adjacent atom.

In one or more embodiments, the electron-transporting host of the emission layer 15 may include at least one of a cyano group, a π electron-deficient nitrogen-containing cyclic group, or a combination thereof.

In one or more embodiments, the electron-transporting host in the emission layer 15 may include at least one cyano group.

In one or more embodiments, the electron-transporting host in the emission layer 15 may include at least one cyano group, at least one π electron deficient nitrogen-containing cyclic group, or a combination thereof.

In one or more embodiments, the host may include an electron-transporting host and a hole-transporting host, wherein the electron-transporting host may include at least one π electron-deficient nitrogen-free cyclic group and at least one electron-transporting moiety, and the hole-transporting host may include at least one π electron-deficient nitrogen-free cyclic group and may not include an electron-transporting moiety.

The term “π electron-deficient nitrogen-containing cyclic group” used herein refers to a cyclic group having at least one *—N═*′ moiety, and for example, may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group; or a condensed cyclic group in of two or more π electron-efficient nitrogen-containing cyclic groups.

Meanwhile, the π electron-deficient nitrogen-free cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a corogen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group, or a condensed cyclic group of two or more π electron-deficient nitrogen-free cyclic groups, but embodiments of the present disclosure are not limited thereto.

In one or more embodiments, the electron-transporting host may be one of the compounds represented by Formula E-1, and

the hole-transporting host may be compounds represented by Formula H-1, but embodiments of the present disclosure are not limited thereto:

[Ar₃₀₁]_{xb11}-[(L₃₀₁)_{xb1}-R₃₀₁]_{xb21} Formula E-1

wherein, in Formula E-1,

Ar₃₀₁ may be a substituted or unsubstituted C₅-C₆₀ carbocyclic group or a substituted or unsubstituted C₁-C₆₀ heterocyclic group,

xb11 may be 1, 2, or 3,

L₃₀₁ may each independently be a single bond, groups represented by one of following chemical structures, a substituted or unsubstituted C₅-C₆₀ carbocyclic group, or a substituted or unsubstituted C₁-C₆₀ heterocyclic group, wherein in the following chemical structures, *, *′, and *″ may each indicate a binding site to an adjacent atom,

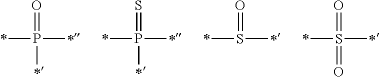

xb1 may be an integer from 1 to 5,

R₃₀₁ may be hydrogen, deuterium, —F, —Cl, —Br, —I, a hydroxyl group, a cyano group, a nitro group, an amidino group, a hydrazino group, a hydrazono group, substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, substituted or unsubstituted C₃-C₁₀ cycloalkyl group, substituted or unsubstituted C₁-C₁₀ heterocycloalkyl group, substituted or unsubstituted C₃-C₁₀ cycloalkenyl group, substituted or unsubstituted C₁-C₁₀ heterocycloalkenyl group, substituted or unsubstituted C₆-C₆₀ aryl group, substituted or unsubstituted C₆-C₆₀ aryloxy group, substituted or unsubstituted C₆-C₆₀ arylthio group, substituted or unsubstituted C₁-C₆₀ heteroaryl group, substituted or unsubstituted monovalent aromatic condensed polycyclic group, substituted or unsubstituted monovalent aromatic heterocondensed polycyclic group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), —S(═O)(Q₃₀₁), —P(═O)(Q₃₀₁)(Q₃₀₂), —P(═S)(Q₃₀₁)(Q₃₀₂), or a combination thereof,

xb21 may be an integer from 1 to 5,

Q₃₀₁ to Q₃₀₃ may each independently be a C₁-C₁₀ alkyl group, a C₁-C₁₀ alkoxy group, a phenyl group, a biphenyl group, a terphenyl group, or a naphthyl group, and

at least one of Condition 1 to Condition 3 is satisfied:

Condition H-1: At least one of Ar₃₀₁, L₃₀₁, and R₃₀₁ in Formula E-1 each independently includes a π electron-deficient nitrogen-containing cyclic group.

Condition H-2: L₃₀₁ in Formula E-1 is a group represented by one of the following chemical structures.

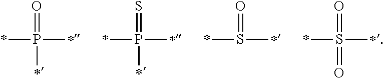

Condition H-3: R₃₀₁ in Formula E-1 is a cyano group, —S(═O)₂(Q₃₀₁), —S(═O)(Q₃₀₁), —P(═O)(Q₃₀₁)(Q₃₀₂), or —P(═S)(Q₃₀₁)(Q₃₀₂).

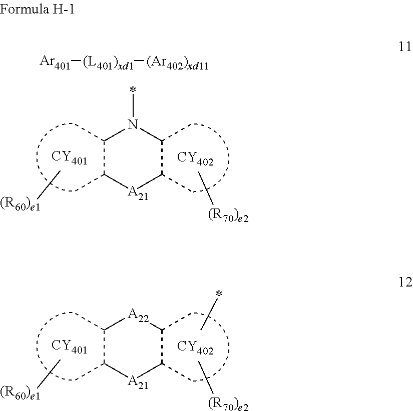

wherein, in Formulae H-1, 11, and 12,

L₄₀₁ may be:

a single bond; or

a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a corogen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with at least one deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₄₀₁)(Q₄₀₂)(Q₄₀₃), or a combination thereof,

xd1 may be an integer from 1 to 10; and when xd1 is 2 or greater, at least two L₄₀₁ groups may be identical to or different from each other,

Ar₄₀₁ may be a group represented by Formulae 11 or 12,

Ar₄₀₂ may be:

a group represented by Formula 11 or 12, a phenyl group, a naphthyl group, a fluorenyl group, a carbazolyl group, a dibenzofuranyl group, a dibenzothiophenyl group, a biphenyl group, a terphenyl group, or a triphenylenyl group; or

a phenyl group, a naphthyl group, a fluorenyl group, a carbazolyl group, a dibenzofuranyl group, a dibenzothiophenyl group, a biphenyl group, a terphenyl group, or a triphenylenyl group, each substituted with at least one deuterium, a hydroxyl group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, a terphenyl group, a triphenylenyl group, or any combination thereof,

CY₄₀₁ and CY₄₀₂ may each independently be a benzene group, a naphthalene group, a fluorene group, a carbazole group, a benzocarbazole group, an indolocarbazole group, a dibenzofuran group, a dibenzothiophene group, a dibenzosilole group, a benzonaphthofuran group, a benzonapthothiophene group, or a benzonaphthosilole group,

A₂₁ may be a single bond, O, S, N(R₅₁), C(R₅₁)(R₅₂), or Si(R₅₁)(R₅₂),

A₂₂ may be a single bond, O, S, N(R₅₃), C(R₅₃)(R₅₄), or Si(R₅₃)(R₅₄),

at least one of A₂₁, A₂₂, or a combination thereof in Formula 12 may not be a single bond,

R₅₁ to R₅₄, R₆₀, and R₇₀ may each independently be:

hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;

a C₁-C₂₀ alkyl group or a C₁-C₂₀ alkoxy group, each substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, or any combination thereof;

a π electron-deficient nitrogen-free cyclic group (e.g., a phenyl group, a naphthyl group, a fluorenyl group, a carbazolyl group, a dibenzofuranyl group, a dibenzothiophenyl group, a biphenyl group, a terphenyl group, and a triphenylenyl group);

a π electron-deficient nitrogen-free cyclic group (e.g., a phenyl group, a naphthyl group, a fluorenyl group, a carbazolyl group, a dibenzofuranyl group, a dibenzothiophenyl group, a biphenyl group, a terphenyl group, and a triphenylenyl group) substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, or any combination thereof,

—Si(Q₄₀₄)(Q₄₀₅)(Q₄₀₆),

e1 and e2 may each independently be an integer from 0 to 10,

wherein Q₄₀₁ to Q₄₀₆ may each independently be hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, a biphenyl group, a terphenyl group, or a triphenylenyl group, and

* indicates a binding site to an adjacent atom.

In some embodiments, in Formula E-1, Ar₃₀₁ and L₃₀₁ may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

at least one of L₃₀₁(s) in the number of xb1 may each independently be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or any combination thereof, and

R₃₀₁ may be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a tetraphenyl group, a naphthyl group, a cyano group-containing phenyl group, a cyano group-containing biphenyl group, a cyano group-containing terphenyl group, a cyano group-containing tetraphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

wherein Q₃₁ to Q₃₃ may each independently be a C₁-C₁₀ alkyl group, a C₁-C₁₀ alkoxy group, a phenyl group, a biphenyl group, a terphenyl group, or a naphthyl group, but embodiments are not limited thereto.

In one or more embodiments, Ar₃₀₁ may be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, and a dibenzothiophene group, each unsubstituted or substituted with at least one deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or any combination thereof; or

a group represented by one of Formulae 5-1 to 5-3 and 6-1 to 6-33, and

L₃₀₁ may be a group represented by one of Formulae 5-1 to 5-3 and 6-1 to 6-33:

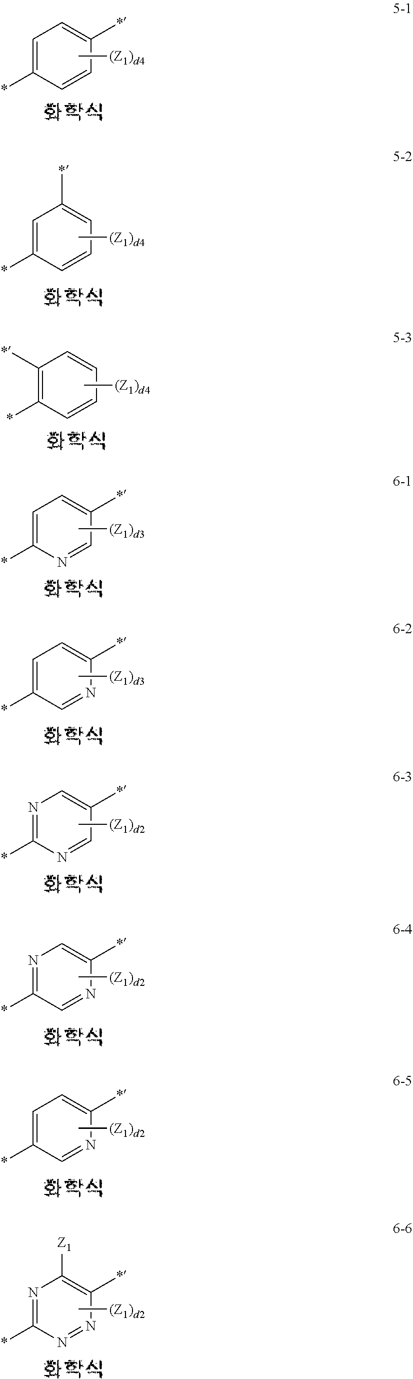

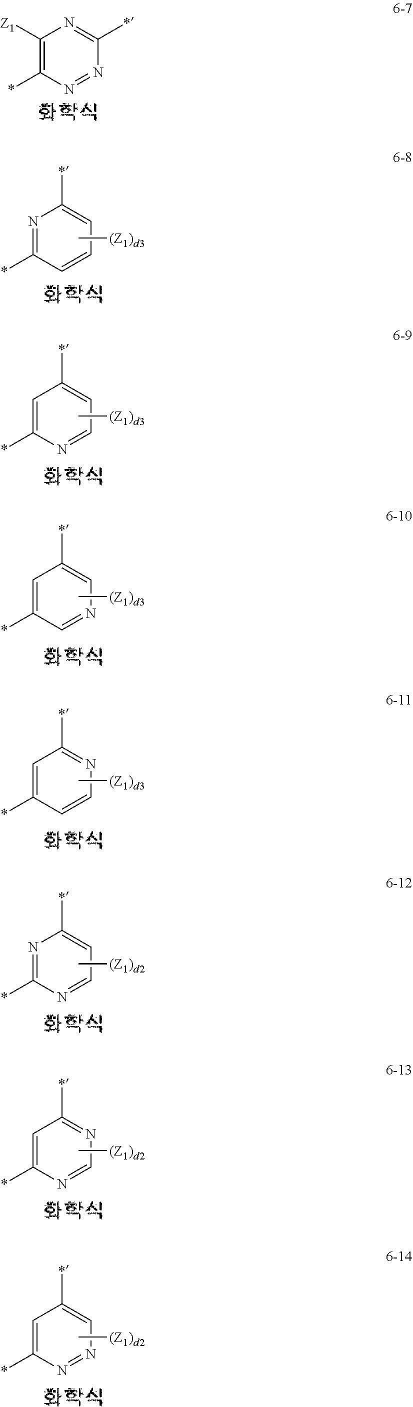

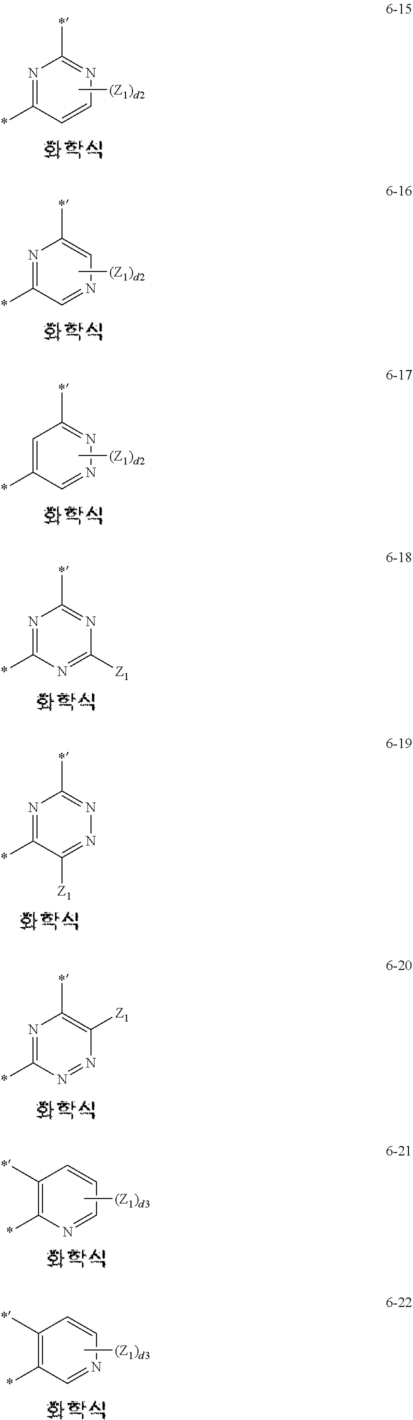

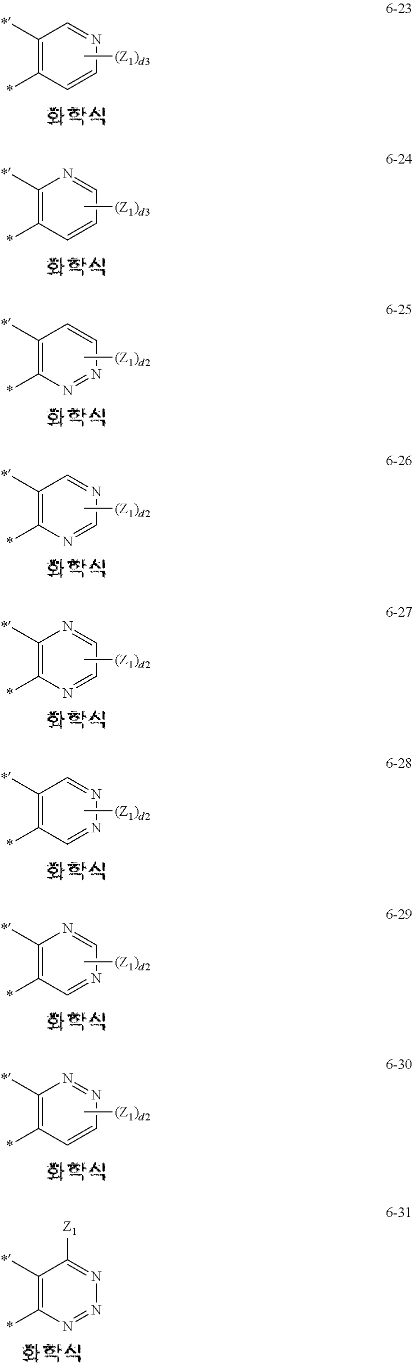

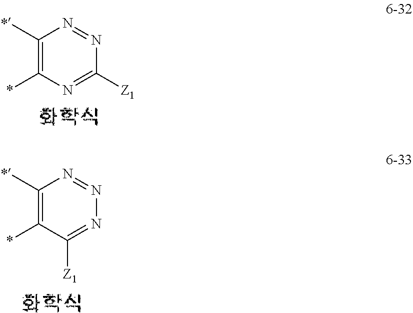

wherein, in Formulae 5-1 to 5-3 and 6-1 to 6-33,

Z₁ may be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

d4 may be 0, 1, 2, 3, or 4,

d3 may be 0, 1, 2, or 3,

d2 may be 0, 1, or 2, and

* and *′ each indicate a binding site to an adjacent atom.

Q₃₁ to Q₃₃ may respectively be understood by referring to the descriptions of Q₃₁ to Q₃₃ provided herein.

In one or more embodiments, L₃₀₁ may be a group represented by one of Formulae 5-2, 5-3 and 6-8 to 6-33.

In one or more embodiments, R₃₀₁ may be a cyano group or a group represented by one of Formula 7-1 to 7-18, and at least one of Ar₄₀₂(s) in the number of xd11 may be a group represented by one of Formulae 7-1 to 7-18, but embodiments of the present disclosure are not limited thereto:

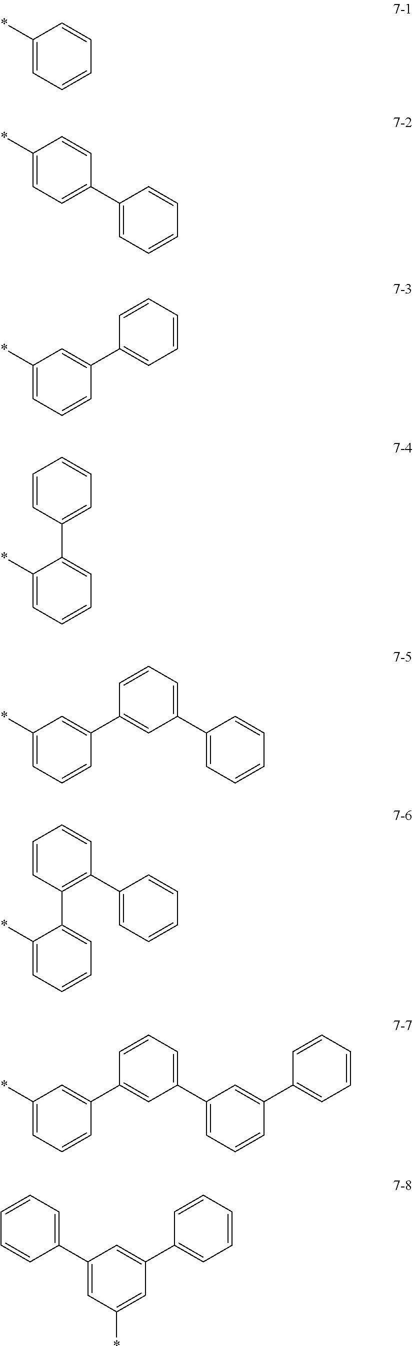

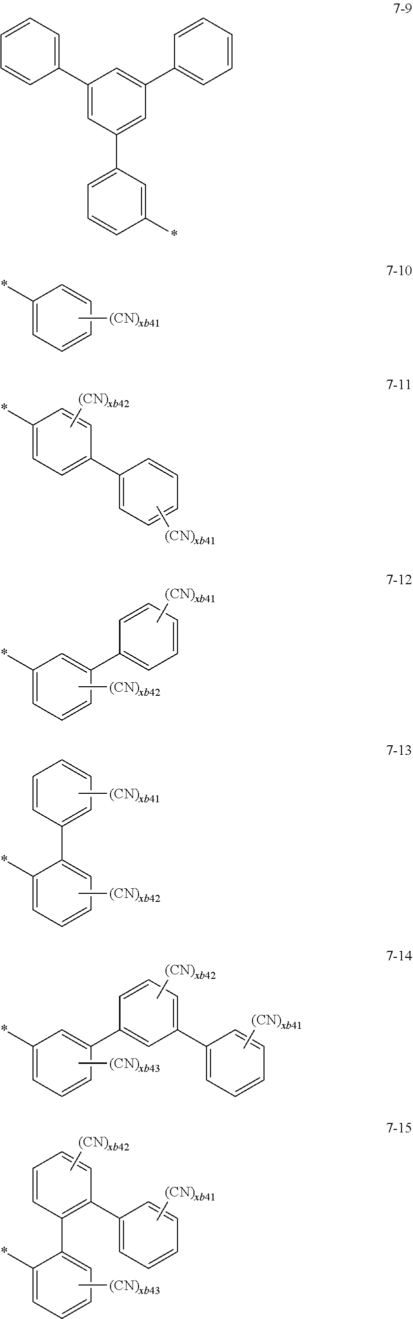

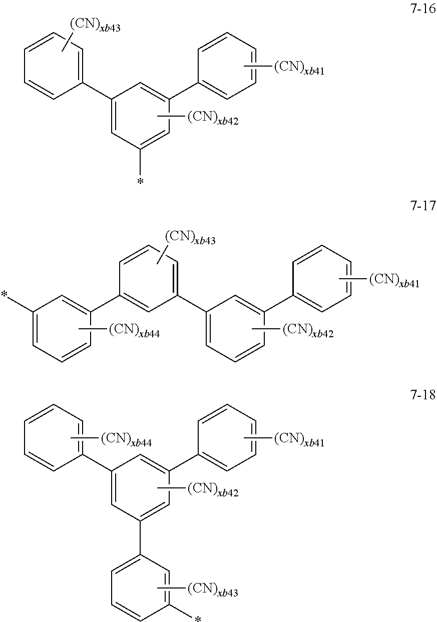

wherein, in Formulae 7-1 to 7-18,

xb41 to xb44 may each be 0, 1, or 2, provided that xb41 in Formula 7-10 may not be 0, xb41+xb42 in Formulae 7-11 to 7-13 may not be 0, xb41+xb42+xb43 in Formulae 7-14 to 7-16 may not be 0, xb41+xb42+xb43+xb44 in Formulae 7-17 and 7-18 may not be 0, and * indicates a binding site to an adjacent atom.

In Formula E-1, at least two Ar₃₀₁(S) may be identical to or different from each other, and at least two L₃₀₁(s) may be identical to or different from each other. In Formula H-1, at least two L₄₀₁(s) may be identical to or different from each other, and at least two Ar₄₀₂(S) may be identical to or different from each other.

In an embodiment, the electron-transporting host includes i) at least one of a cyano group, a pyrimidine group, a pyrazine group, a triazine group, or any combination thereof, and ii) a triphenylene group, and the hole-transporting host may include a carbazole group.

In one or more embodiments, the electron-transporting host may include at least one cyano group.

The electron transporting host may be, for example, a compound of Groups HE1 to HE7, but embodiments are not limited thereto:

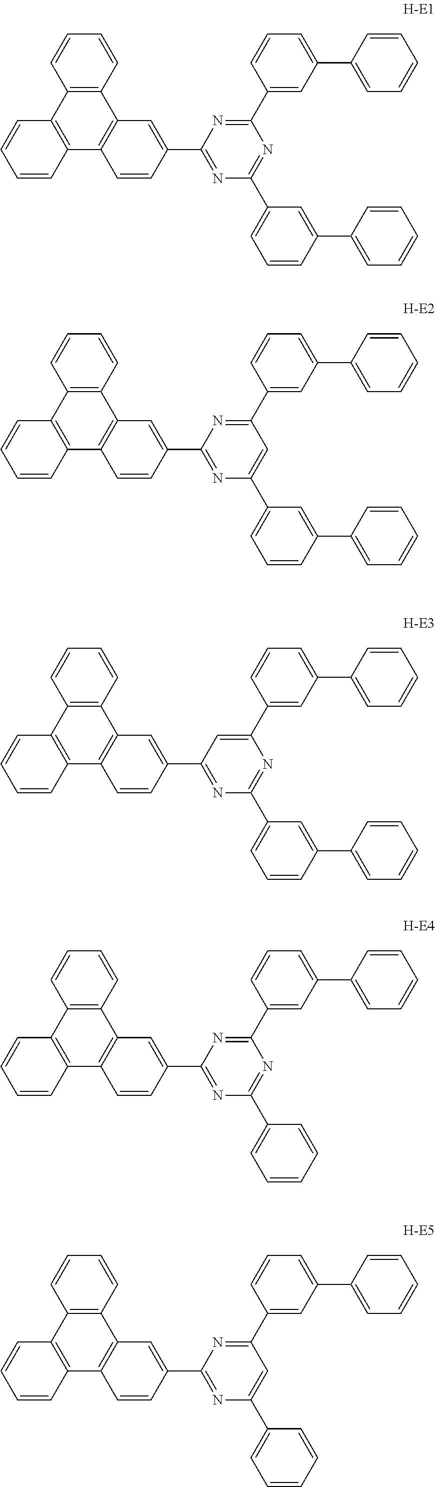 [SMILES: c1ccc(-c2cccc(-c3nc(-c4cccc(-c5ccccc5)c4)nc(-c4ccc5c6ccccc6c6ccccc6c5c4)n3)c2)cc1 ; c1ccc(-c2cccc(-c3cc(-c4cccc(-c5ccccc5)c4)nc(-c4ccc5c6ccccc6c6ccccc6c5c4)n3)c2)cc1 ; c1ccc(-c2cccc(-c3cc(-c4ccc5c6ccccc6c6ccccc6c5c4)nc(-c4cccc(-c5ccccc5)c4)n3)c2)cc1 ; c1ccc(-c2cccc(-c3nc(-c4ccccc4)nc(-c4ccc5c6ccccc6c6ccccc6c5c4)n3)c2)cc1 ; c1ccc(-c2cccc(-c3cc(-c4ccccc4)nc(-c4ccc5c6ccccc6c6ccccc6c5c4)n3)c2)cc1]

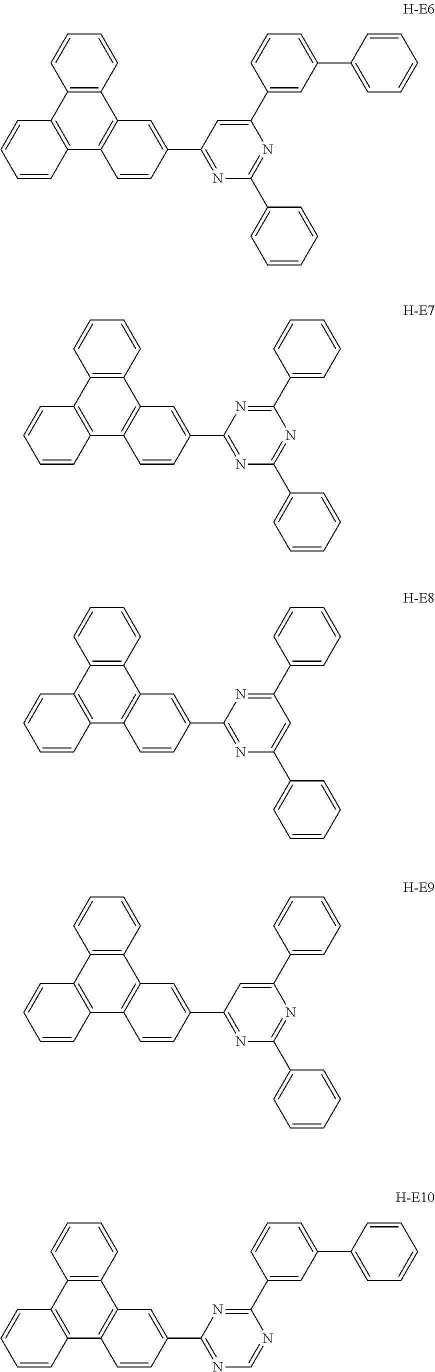 [SMILES: c1ccc(-c2cccc(-c3cc(-c4ccc5c6ccccc6c6ccccc6c5c4)nc(-c4ccccc4)n3)c2)cc1 ; c1ccc(-c2nc(-c3ccccc3)nc(-c3ccc4c5ccccc5c5ccccc5c4c3)n2)cc1 ; c1ccc(-c2cc(-c3ccccc3)nc(-c3ccc4c5ccccc5c5ccccc5c4c3)n2)cc1 ; c1ccc(-c2cc(-c3ccc4c5ccccc5c5ccccc5c4c3)nc(-c3ccccc3)n2)cc1 ; c1ccc(-c2cccc(-c3ncnc(-c4ccc5c6ccccc6c6ccccc6c5c4)n3)c2)cc1]

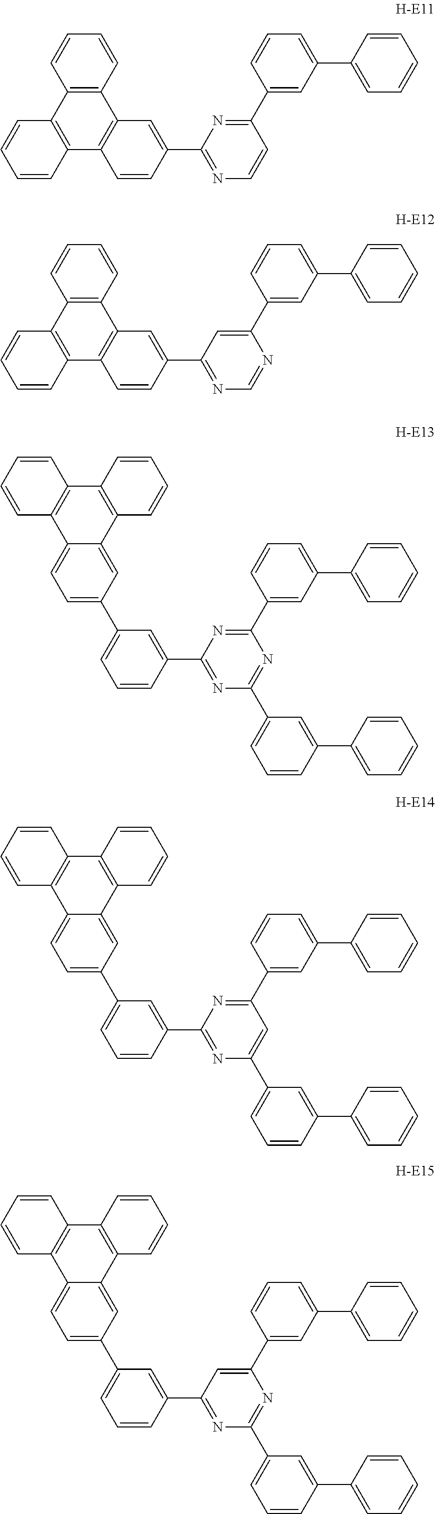 [SMILES: c1ccc(-c2cccc(-c3ccnc(-c4ccc5c6ccccc6c6ccccc6c5c4)n3)c2)cc1 ; c1ccc(-c2cccc(-c3cc(-c4ccc5c6ccccc6c6ccccc6c5c4)ncn3)c2)cc1 ; c1ccc(-c2cccc(-c3nc(-c4cccc(-c5ccccc5)c4)nc(-c4cccc(-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)nc(-c4cccc(-c5ccccc5)c4)n3)c2)cc1]

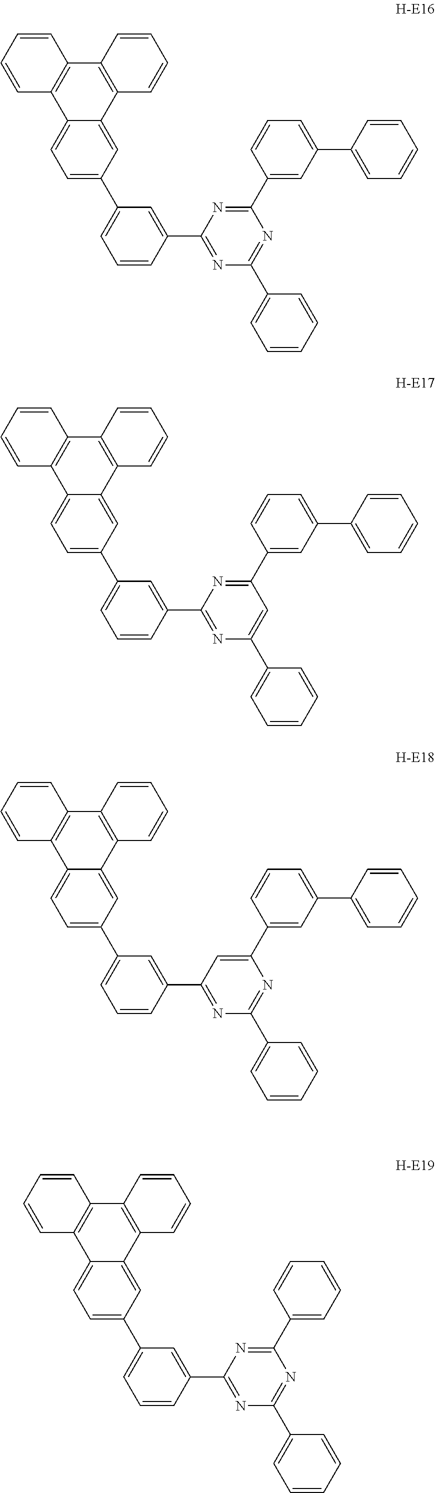 [SMILES: c1ccc(-c2cccc(-c3nc(-c4ccccc4)nc(-c4cccc(-c5ccc6c7ccccc7c7ccccc7c6c5)c4)n3)c2)cc1 ; c1ccc(-c2cccc(-c3cc(-c4ccccc4)nc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)nc(-c4ccccc4)n3)c2)cc1 ; c1ccc(-c2nc(-c3ccccc3)nc(-c3cccc(-c4ccc5c6ccccc6c6ccccc6c5c4)c3)n2)cc1]

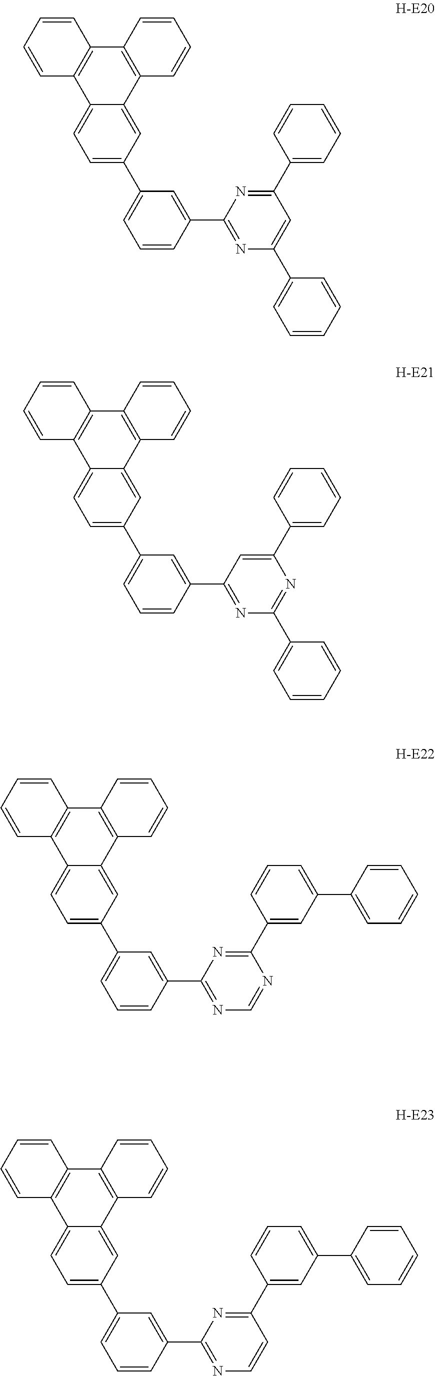 [SMILES: c1ccc(-c2cc(-c3ccccc3)nc(-c3cccc(-c4ccc5c6ccccc6c6ccccc6c5c4)c3)n2)cc1 ; c1ccc(-c2cc(-c3cccc(-c4ccc5c6ccccc6c6ccccc6c5c4)c3)nc(-c3ccccc3)n2)cc1 ; c1ccc(-c2cccc(-c3ncnc(-c4cccc(-c5ccc6c7ccccc7c7ccccc7c6c5)c4)n3)c2)cc1 ; c1ccc(-c2cccc(-c3ccnc(-c4cccc(-c5ccc6c7ccccc7c7ccccc7c6c5)c4)n3)c2)cc1]

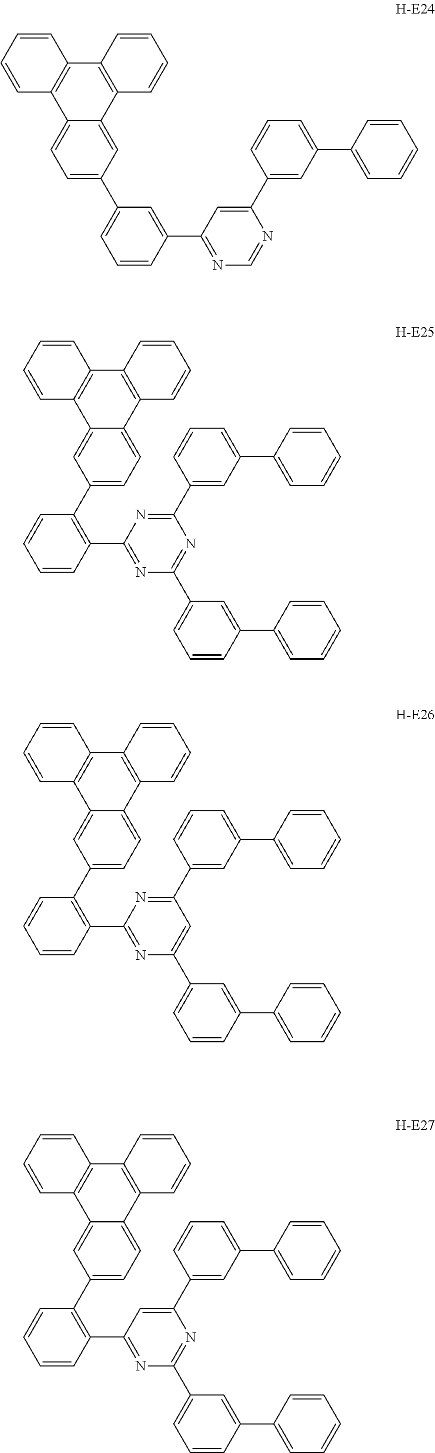 [SMILES: c1ccc(-c2cccc(-c3cc(-c4cccc(-c5ccc6c7ccccc7c7ccccc7c6c5)c4)ncn3)c2)cc1 ; c1ccc(-c2cccc(-c3nc(-c4cccc(-c5ccccc5)c4)nc(-c4ccccc4-c4ccc5c6ccccc6c6ccccc6c5c4)n3)c2)cc1 ; c1ccc(-c2cccc(-c3cc(-c4cccc(-c5ccccc5)c4)nc(-c4ccccc4-c4ccc5c6ccccc6c6ccccc6c5c4)n3)c2)cc1 ; c1ccc(-c2cccc(-c3cc(-c4ccccc4-c4ccc5c6ccccc6c6ccccc6c5c4)nc(-c4cccc(-c5ccccc5)c4)n3)c2)cc1]

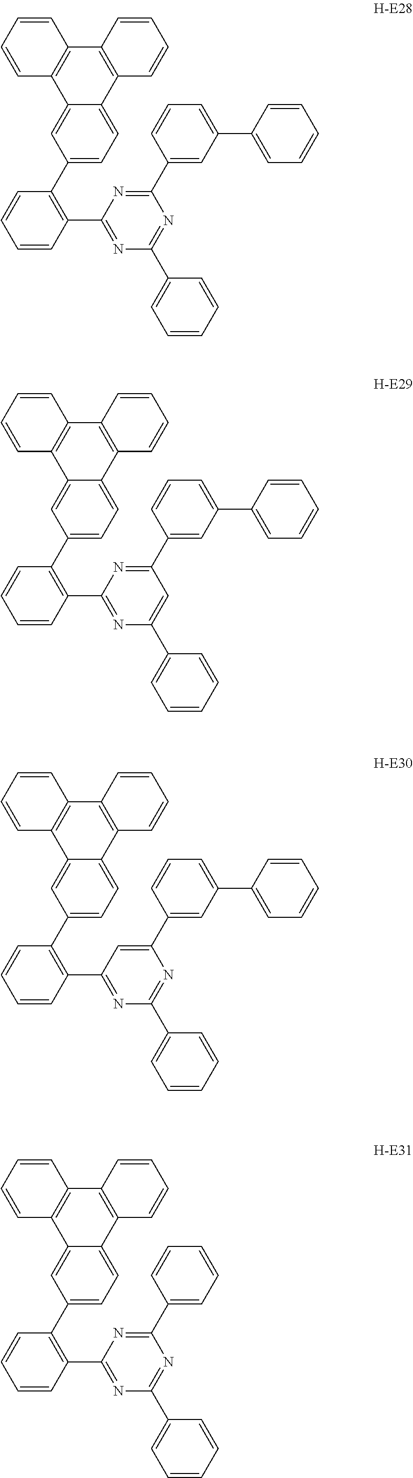 [SMILES: c1ccc(-c2cccc(-c3nc(-c4ccccc4)nc(-c4ccccc4-c4ccc5c6ccccc6c6ccccc6c5c4)n3)c2)cc1 ; c1ccc(-c2cccc(-c3cc(-c4ccccc4)nc(-c4ccccc4-c4ccc5c6ccccc6c6ccccc6c5c4)n3)c2)cc1 ; c1ccc(-c2cccc(-c3cc(-c4ccccc4-c4ccc5c6ccccc6c6ccccc6c5c4)nc(-c4ccccc4)n3)c2)cc1 ; c1ccc(-c2nc(-c3ccccc3)nc(-c3ccccc3-c3ccc4c5ccccc5c5ccccc5c4c3)n2)cc1]

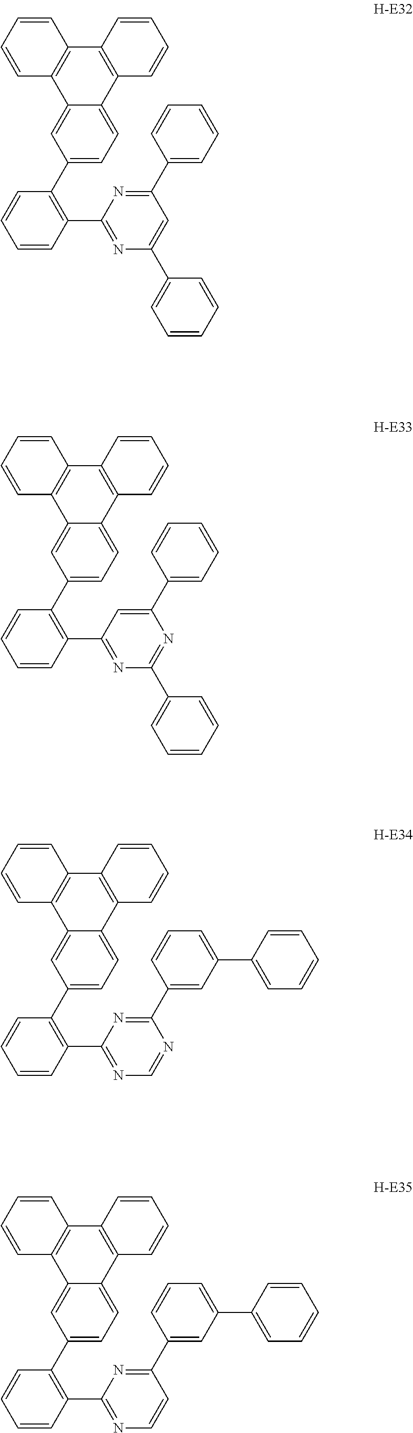 [SMILES: c1ccc(-c2cc(-c3ccccc3)nc(-c3ccccc3-c3ccc4c5ccccc5c5ccccc5c4c3)n2)cc1 ; c1ccc(-c2cc(-c3ccccc3-c3ccc4c5ccccc5c5ccccc5c4c3)nc(-c3ccccc3)n2)cc1 ; c1ccc(-c2cccc(-c3ncnc(-c4ccccc4-c4ccc5c6ccccc6c6ccccc6c5c4)n3)c2)cc1 ; c1ccc(-c2cccc(-c3ccnc(-c4ccccc4-c4ccc5c6ccccc6c6ccccc6c5c4)n3)c2)cc1]

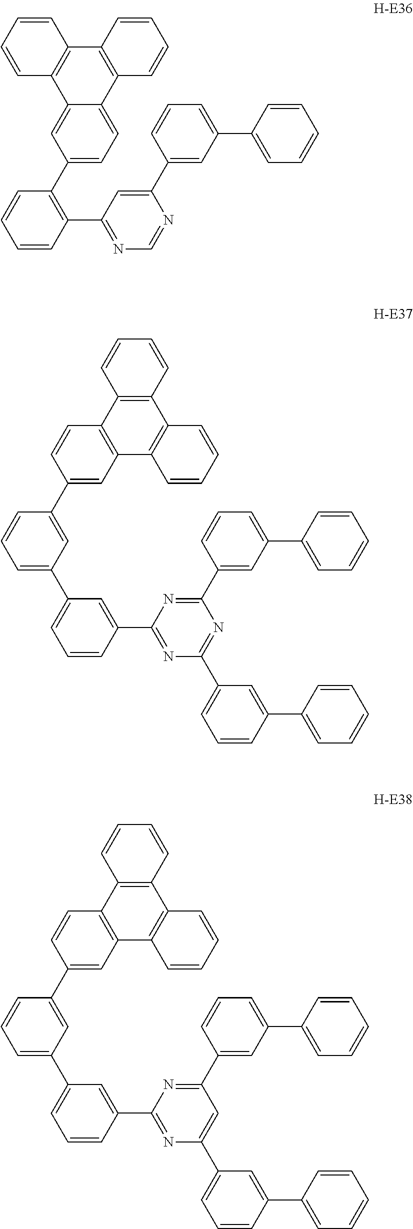 [SMILES: c1ccc(-c2cccc(-c3cc(-c4ccccc4-c4ccc5c6ccccc6c6ccccc6c5c4)ncn3)c2)cc1 ; c1ccc(-c2cccc(-c3nc(-c4cccc(-c5ccccc5)c4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5ccccc5)c4)nc(-c4cccc(-c5cccc(-c6ccc7c8ccccc8c8ccccc8c7c6)c5)c4)n3)c2)cc1]

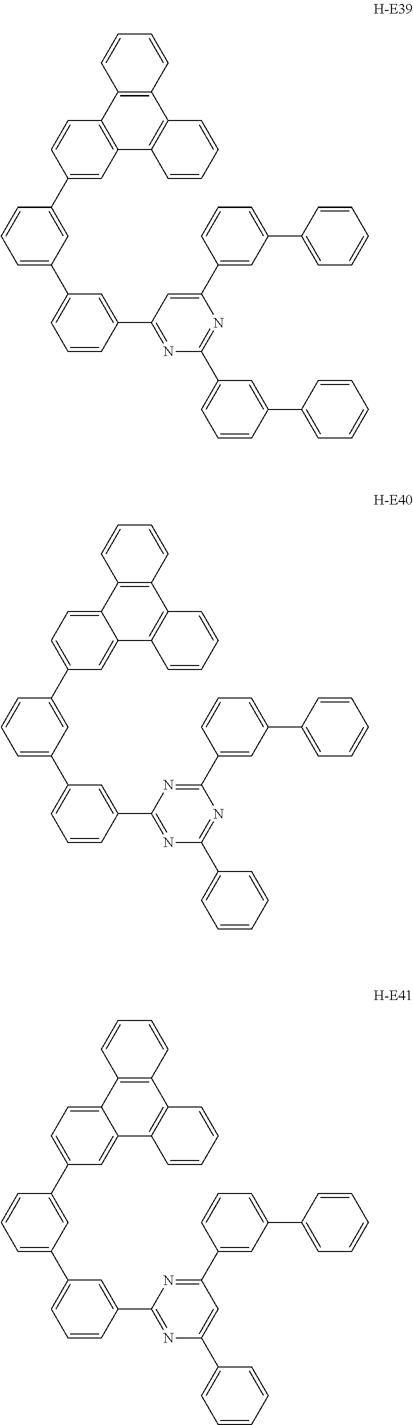 [SMILES: c1ccc(-c2cccc(-c3cc(-c4cccc(-c5cccc(-c6ccc7c8ccccc8c8ccccc8c7c6)c5)c4)nc(-c4cccc(-c5ccccc5)c4)n3)c2)cc1 ; c1ccc(-c2cccc(-c3nc(-c4ccccc4)nc(-c4cccc(-c5cccc(-c6ccc7c8ccccc8c8ccccc8c7c6)c5)c4)n3)c2)cc1 ; c1ccc(-c2cccc(-c3cc(-c4ccccc4)nc(-c4cccc(-c5cccc(-c6ccc7c8ccccc8c8ccccc8c7c6)c5)c4)n3)c2)cc1]

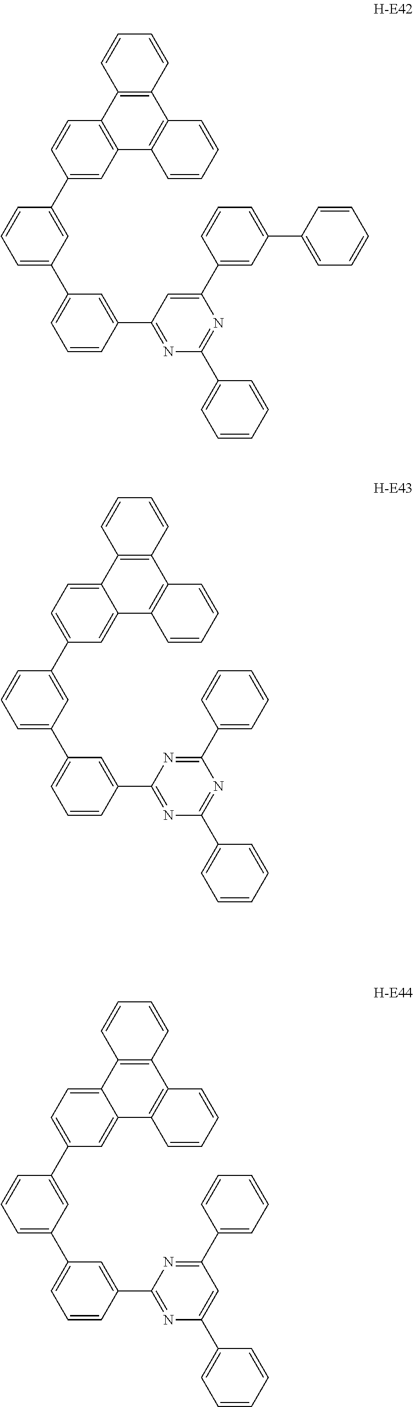 [SMILES: c1ccc(-c2cccc(-c3cc(-c4cccc(-c5cccc(-c6ccc7c8ccccc8c8ccccc8c7c6)c5)c4)nc(-c4ccccc4)n3)c2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2cc(-c3ccccc3)nc(-c3cccc(-c4cccc(-c5ccc6c7ccccc7c7ccccc7c6c5)c4)c3)n2)cc1]

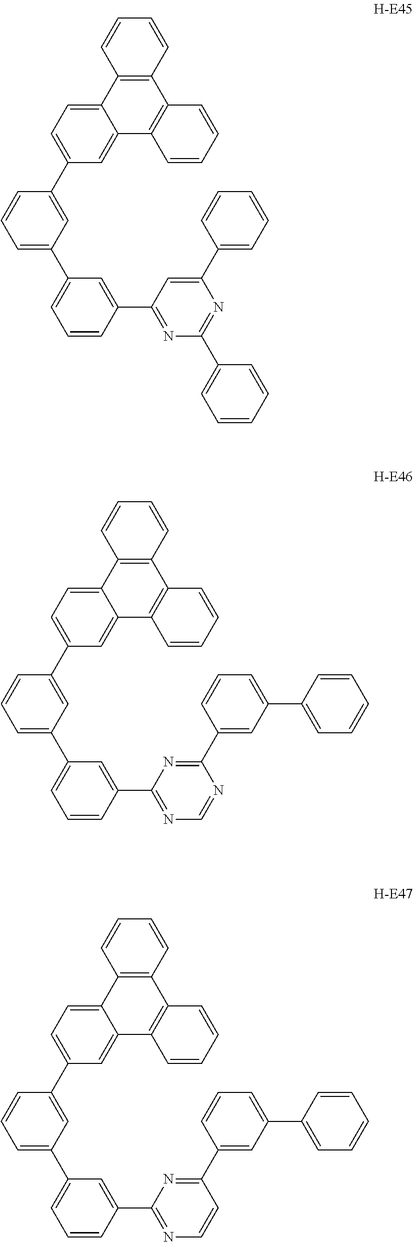 [SMILES: c1ccc(-c2cc(-c3cccc(-c4cccc(-c5ccc6c7ccccc7c7ccccc7c6c5)c4)c3)nc(-c3ccccc3)n2)cc1 ; c1ccc(-c2cccc(-c3ncnc(-c4cccc(-c5cccc(-c6ccc7c8ccccc8c8ccccc8c7c6)c5)c4)n3)c2)cc1 ; c1ccc(-c2cccc(-c3ccnc(-c4cccc(-c5cccc(-c6ccc7c8ccccc8c8ccccc8c7c6)c5)c4)n3)c2)cc1]

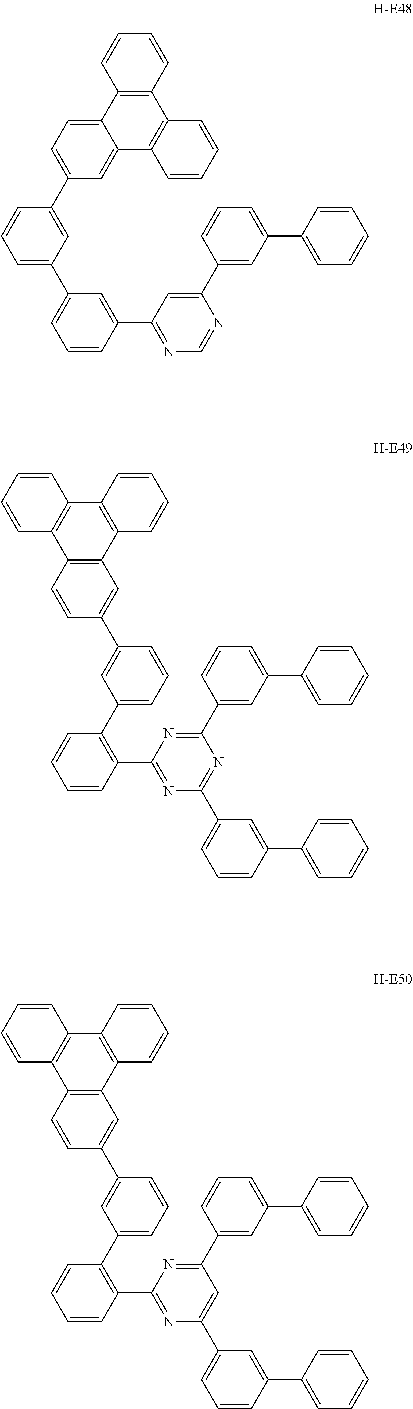 [SMILES: c1ccc(-c2cccc(-c3cc(-c4cccc(-c5cccc(-c6ccc7c8ccccc8c8ccccc8c7c6)c5)c4)ncn3)c2)cc1 ; c1ccc(-c2cccc(-c3nc(-c4cccc(-c5ccccc5)c4)nc(-c4ccccc4-c4cccc(-c5ccc6c7ccccc7c7ccccc7c6c5)c4)n3)c2)cc1 ; c1ccc(-c2cccc(-c3cc(-c4cccc(-c5ccccc5)c4)nc(-c4ccccc4-c4cccc(-c5ccc6c7ccccc7c7ccccc7c6c5)c4)n3)c2)cc1]

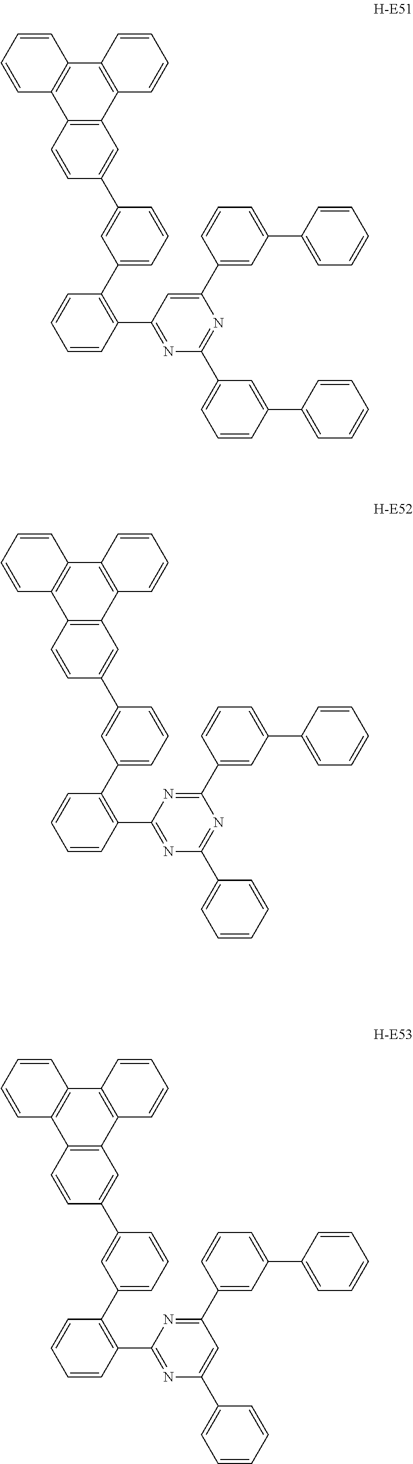 [SMILES: c1ccc(-c2cccc(-c3cc(-c4ccccc4-c4cccc(-c5ccc6c7ccccc7c7ccccc7c6c5)c4)nc(-c4cccc(-c5ccccc5)c4)n3)c2)cc1 ; c1ccc(-c2cccc(-c3nc(-c4ccccc4)nc(-c4ccccc4-c4cccc(-c5ccc6c7ccccc7c7ccccc7c6c5)c4)n3)c2)cc1 ; c1ccc(-c2cccc(-c3cc(-c4ccccc4)nc(-c4ccccc4-c4cccc(-c5ccc6c7ccccc7c7ccccc7c6c5)c4)n3)c2)cc1]

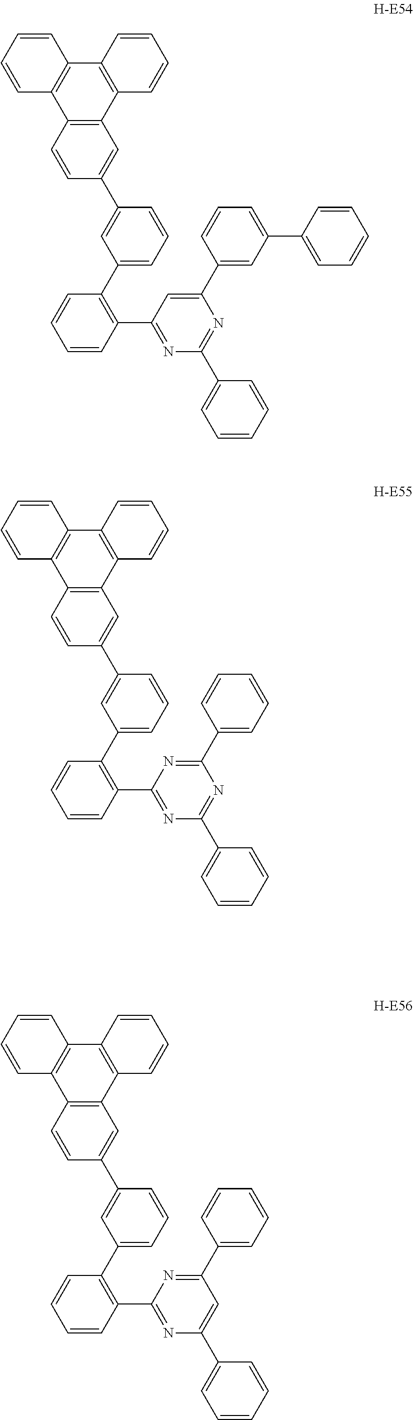 [SMILES: c1ccc(-c2cccc(-c3cc(-c4ccccc4-c4cccc(-c5ccc6c7ccccc7c7ccccc7c6c5)c4)nc(-c4ccccc4)n3)c2)cc1 ; c1ccc(-c2nc(-c3ccccc3)nc(-c3ccccc3-c3cccc(-c4ccc5c6ccccc6c6ccccc6c5c4)c3)n2)cc1 ; c1ccc(-c2cc(-c3ccccc3)nc(-c3ccccc3-c3cccc(-c4ccc5c6ccccc6c6ccccc6c5c4)c3)n2)cc1]

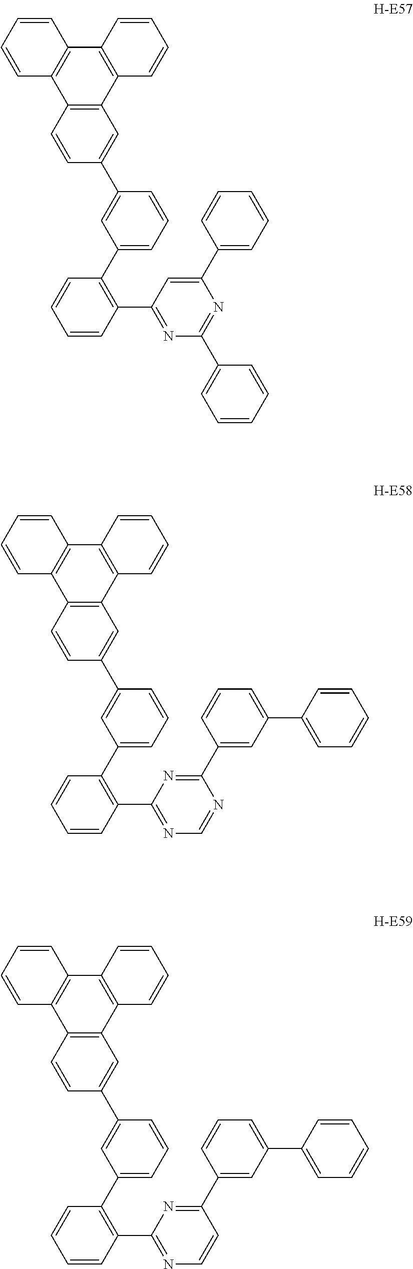 [SMILES: c1ccc(-c2cc(-c3ccccc3-c3cccc(-c4ccc5c6ccccc6c6ccccc6c5c4)c3)nc(-c3ccccc3)n2)cc1 ; c1ccc(-c2cccc(-c3ncnc(-c4ccccc4-c4cccc(-c5ccc6c7ccccc7c7ccccc7c6c5)c4)n3)c2)cc1 ; c1ccc(-c2cccc(-c3ccnc(-c4ccccc4-c4cccc(-c5ccc6c7ccccc7c7ccccc7c6c5)c4)n3)c2)cc1]

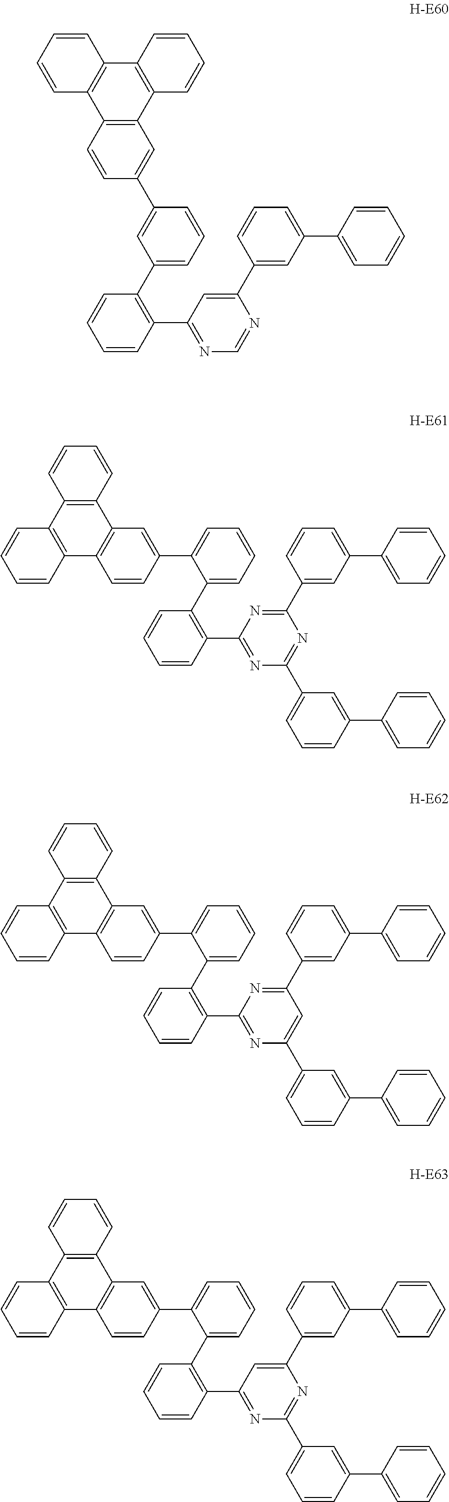 [SMILES: c1ccc(-c2cccc(-c3cc(-c4ccccc4-c4cccc(-c5ccc6c7ccccc7c7ccccc7c6c5)c4)ncn3)c2)cc1 ; c1ccc(-c2cccc(-c3nc(-c4cccc(-c5ccccc5)c4)nc(-c4ccccc4-c4ccccc4-c4ccc5c6ccccc6c6ccccc6c5c4)n3)c2)cc1 ; c1ccc(-c2cccc(-c3cc(-c4cccc(-c5ccccc5)c4)nc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)nc(-c4cccc(-c5ccccc5)c4)n3)c2)cc1]

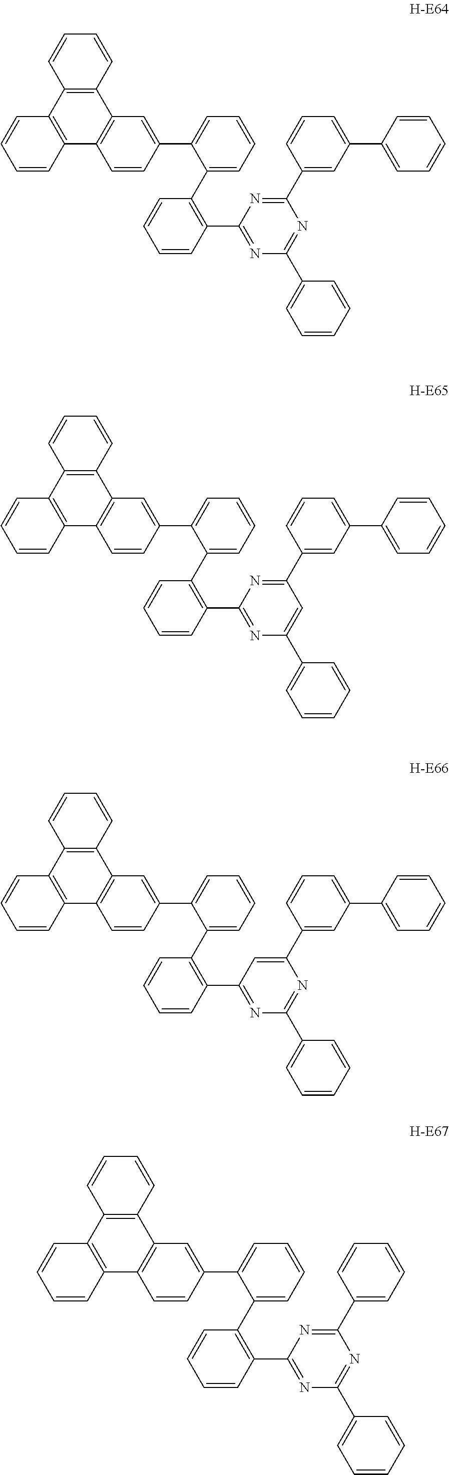 [SMILES: c1ccc(-c2cccc(-c3nc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)nc(-c4ccccc4)n3)c2)cc1 ; c1ccc(-c2nc(-c3ccccc3)nc(-c3ccccc3-c3ccccc3-c3ccc4c5ccccc5c5ccccc5c4c3)n2)cc1]

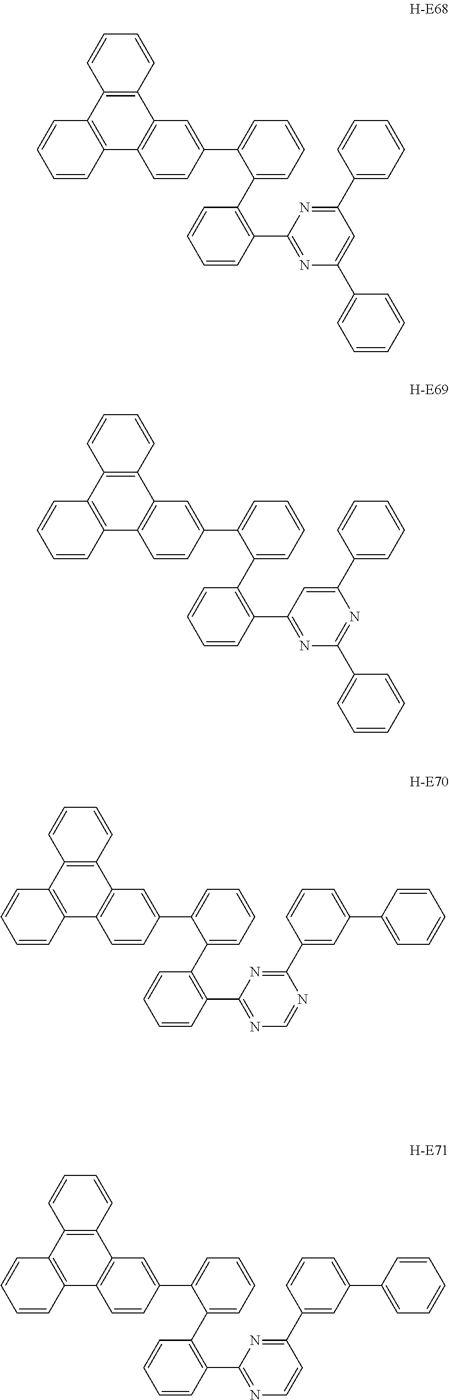 [SMILES: c1ccc(-c2cc(-c3ccccc3)nc(-c3ccccc3-c3ccccc3-c3ccc4c5ccccc5c5ccccc5c4c3)n2)cc1 ; c1ccc(-c2cc(-c3ccccc3-c3ccccc3-c3ccc4c5ccccc5c5ccccc5c4c3)nc(-c3ccccc3)n2)cc1 ; c1ccc(-c2cccc(-c3ncnc(-c4ccccc4-c4ccccc4-c4ccc5c6ccccc6c6ccccc6c5c4)n3)c2)cc1 ; c1ccc(-c2cccc(-c3ccnc(-c4ccccc4-c4ccccc4-c4ccc5c6ccccc6c6ccccc6c5c4)n3)c2)cc1]

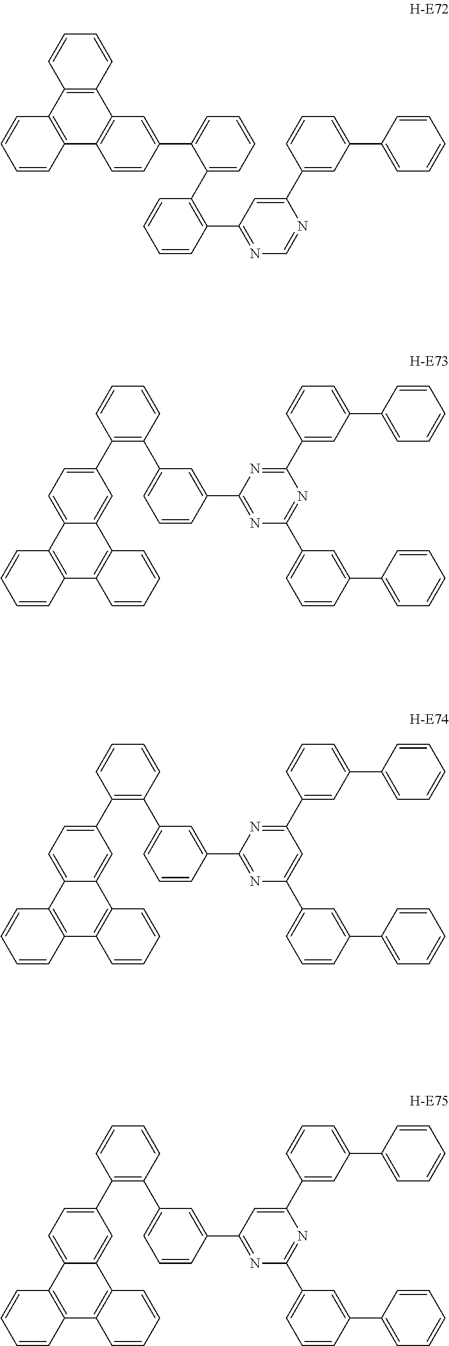 [SMILES: c1ccc(-c2cccc(-c3cc(-c4ccccc4-c4ccccc4-c4ccc5c6ccccc6c6ccccc6c5c4)ncn3)c2)cc1 ; c1ccc(-c2cccc(-c3nc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)nc(-c4cccc(-c5ccccc5)c4)n3)c2)cc1]

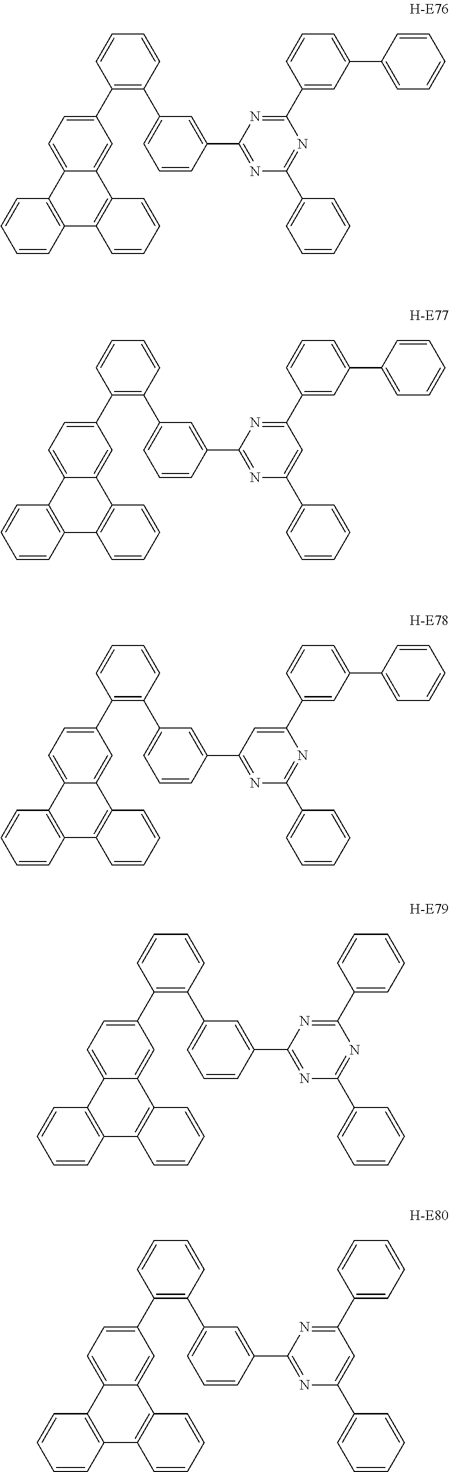 [SMILES: c1ccc(-c2cccc(-c3nc(-c4ccccc4)nc(-c4cccc(-c5ccccc5-c5ccc6c7ccccc7c7ccccc7c6c5)c4)n3)c2)cc1 ; c1ccc(-c2cccc(-c3cc(-c4ccccc4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)nc(-c4ccccc4)n3)c2)cc1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3ccccc3)nc(-c3cccc(-c4ccccc4-c4ccc5c6ccccc6c6ccccc6c5c4)c3)n2)cc1]

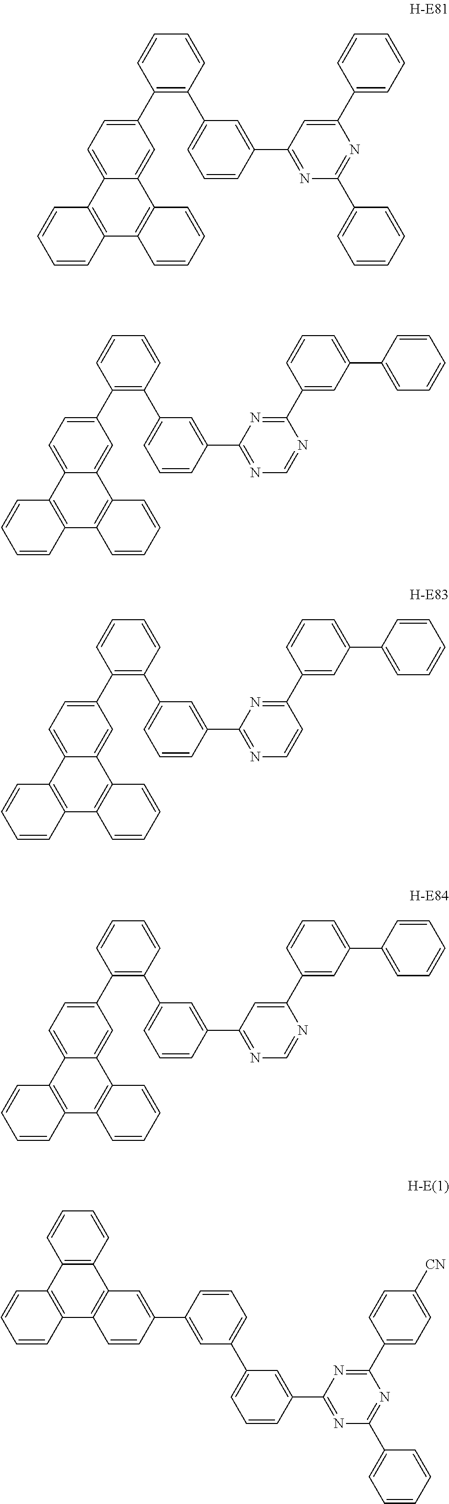 [SMILES: c1ccc(-c2cc(-c3cccc(-c4ccccc4-c4ccc5c6ccccc6c6ccccc6c5c4)c3)nc(-c3ccccc3)n2)cc1 ; c1ccc(-c2cccc(-c3ncnc(-c4cccc(-c5ccccc5-c5ccc6c7ccccc7c7ccccc7c6c5)c4)n3)c2)cc1 ; c1ccc(-c2cccc(-c3ccnc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)ncn3)c2)cc1 ; N#Cc1ccc(-c2nc(-c3ccccc3)nc(-c3cccc(-c4cccc(-c5ccc6c7ccccc7c7ccccc7c6c5)c4)c3)n2)cc1]

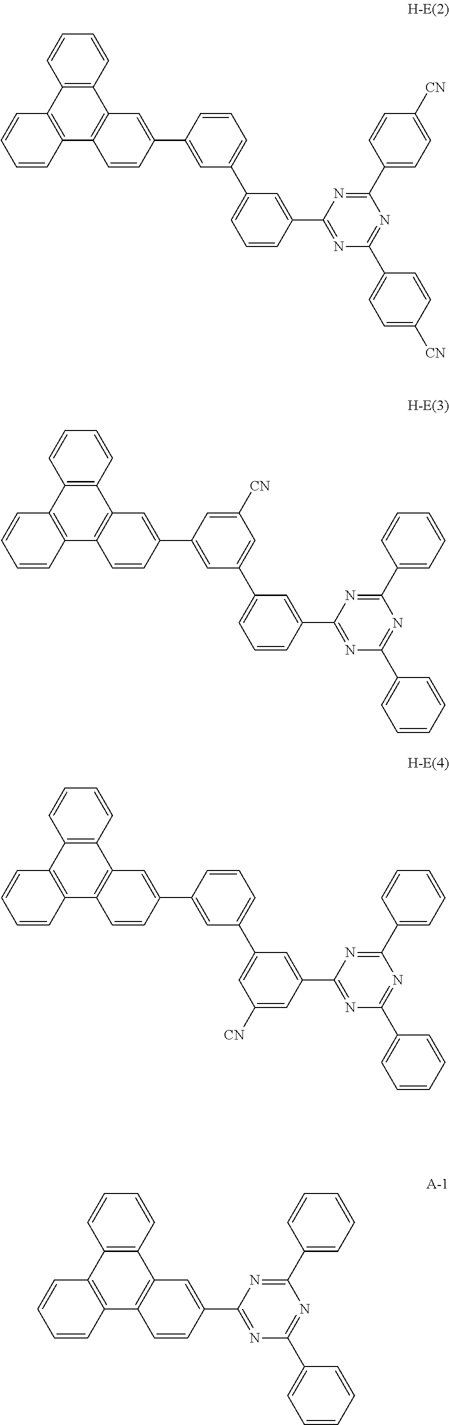

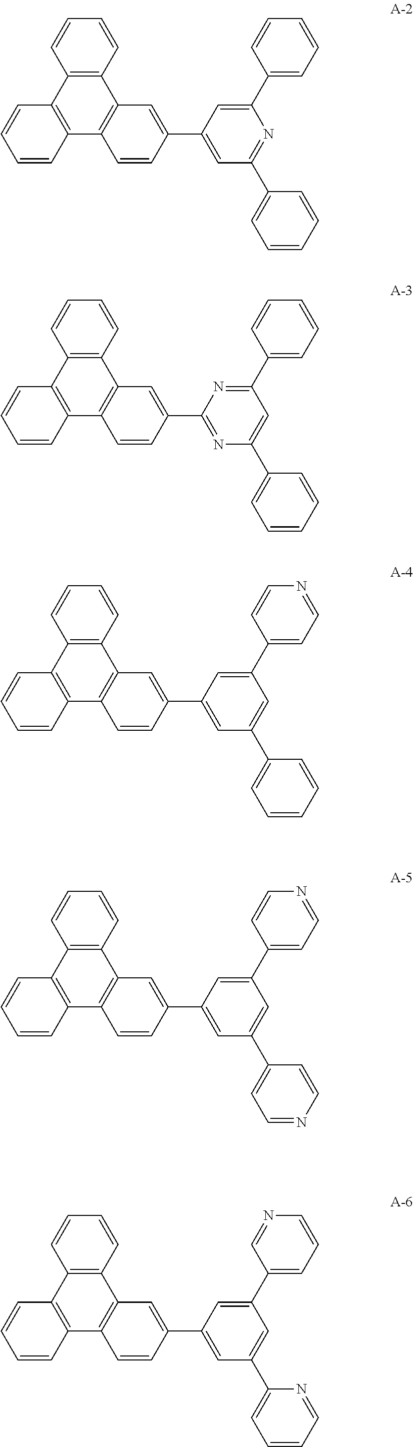 [SMILES: c1ccc(-c2cc(-c3ccc4c5ccccc5c5ccccc5c4c3)cc(-c3ccccc3)n2)cc1 ; c1ccc(-c2cc(-c3ccccc3)nc(-c3ccc4c5ccccc5c5ccccc5c4c3)n2)cc1 ; c1ccc(-c2cc(-c3ccncc3)cc(-c3ccc4c5ccccc5c5ccccc5c4c3)c2)cc1 ; c1ccc2c(c1)c1ccccc1c1cc(-c3cc(-c4ccncc4)cc(-c4ccncc4)c3)ccc21 ; c1ccc(-c2cc(-c3cccnc3)cc(-c3ccc4c5ccccc5c5ccccc5c4c3)c2)nc1]

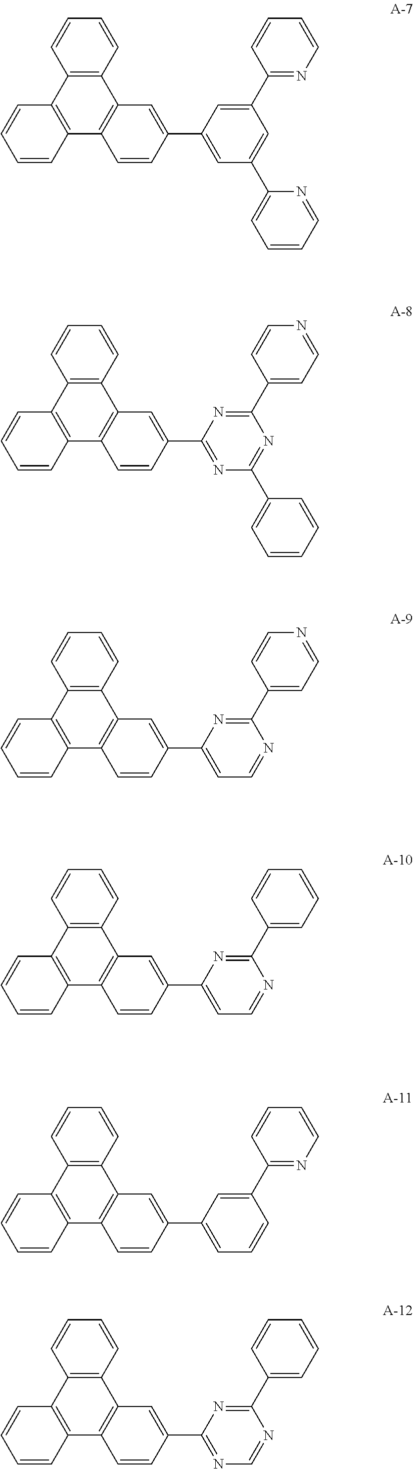 [SMILES: c1ccc(-c2cc(-c3ccc4c5ccccc5c5ccccc5c4c3)cc(-c3ccccn3)c2)nc1 ; c1ccc(-c2nc(-c3ccncc3)nc(-c3ccc4c5ccccc5c5ccccc5c4c3)n2)cc1 ; c1ccc2c(c1)c1ccccc1c1cc(-c3ccnc(-c4ccncc4)n3)ccc21 ; c1ccc(-c2nccc(-c3ccc4c5ccccc5c5ccccc5c4c3)n2)cc1 ; c1ccc(-c2cccc(-c3ccc4c5ccccc5c5ccccc5c4c3)c2)nc1 ; c1ccc(-c2ncnc(-c3ccc4c5ccccc5c5ccccc5c4c3)n2)cc1]

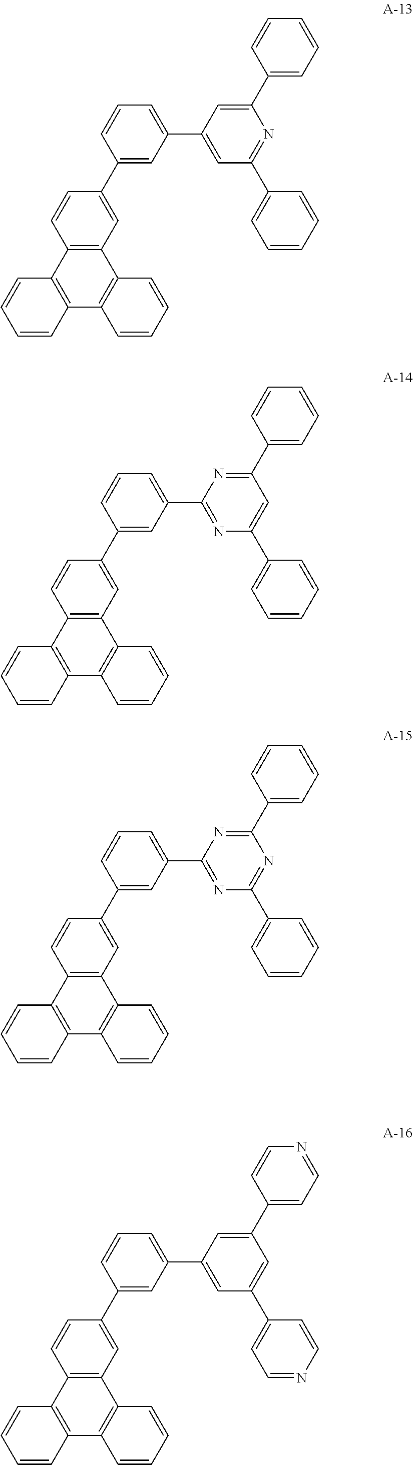 [SMILES: c1ccc(-c2cc(-c3cccc(-c4ccc5c6ccccc6c6ccccc6c5c4)c3)cc(-c3ccccc3)n2)cc1 ; c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1cc(-c2cc(-c3ccncc3)cc(-c3ccncc3)c2)cc(-c2ccc3c4ccccc4c4ccccc4c3c2)c1]

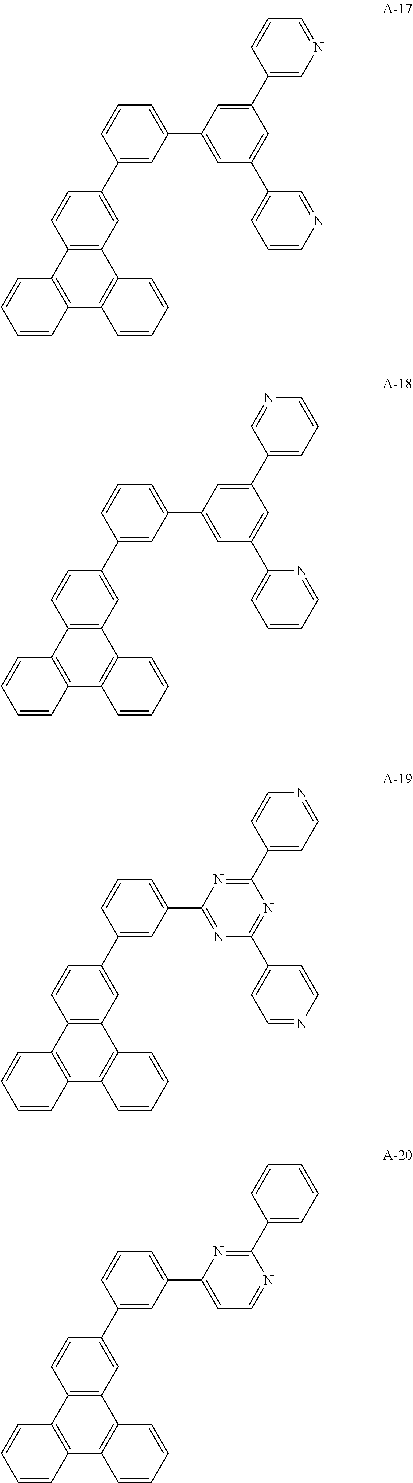 [SMILES: c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1ccc(-c2nccc(-c3cccc(-c4ccc5c6ccccc6c6ccccc6c5c4)c3)n2)cc1]

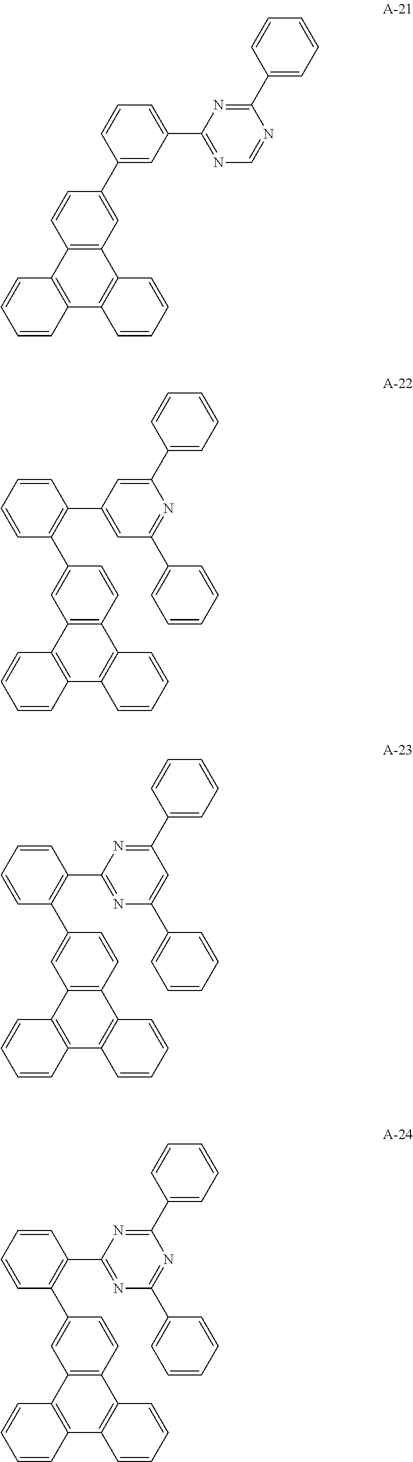 [SMILES: c1ccc(-c2ncnc(-c3cccc(-c4ccc5c6ccccc6c6ccccc6c5c4)c3)n2)cc1 ; c1ccc(-c2cc(-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1]

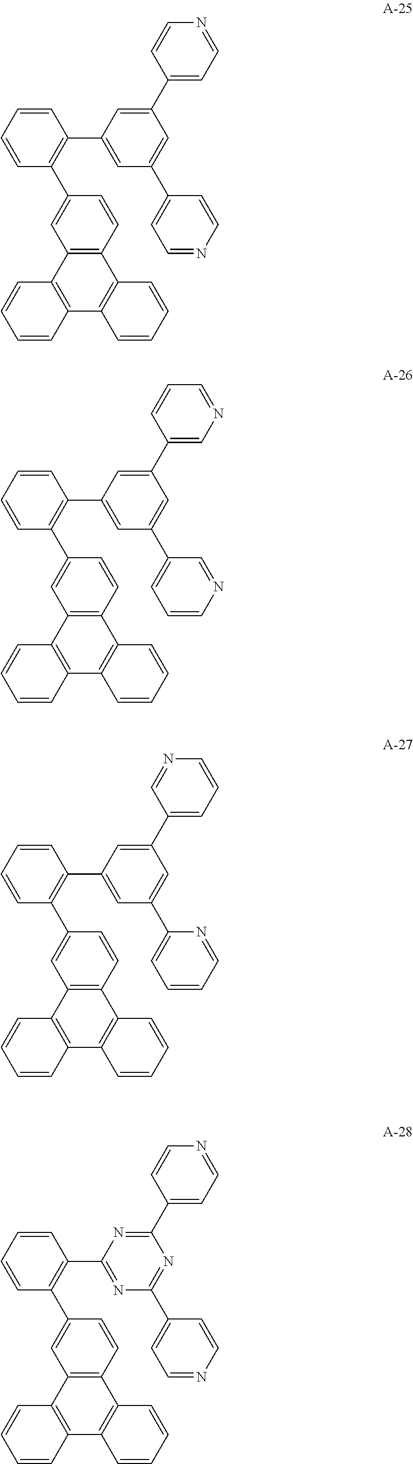 [SMILES: c1ccc(-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccc4c5ccccc5c5ccccc5c4c3)c2)c1 ; c1ccc(-c2cc(-c3cccnc3)cc(-c3ccccc3-c3ccc4c5ccccc5c5ccccc5c4c3)c2)nc1 ; c1ccc(-c2nc(-c3ccncc3)nc(-c3ccncc3)n2)c(-c2ccc3c4ccccc4c4ccccc4c3c2)c1]

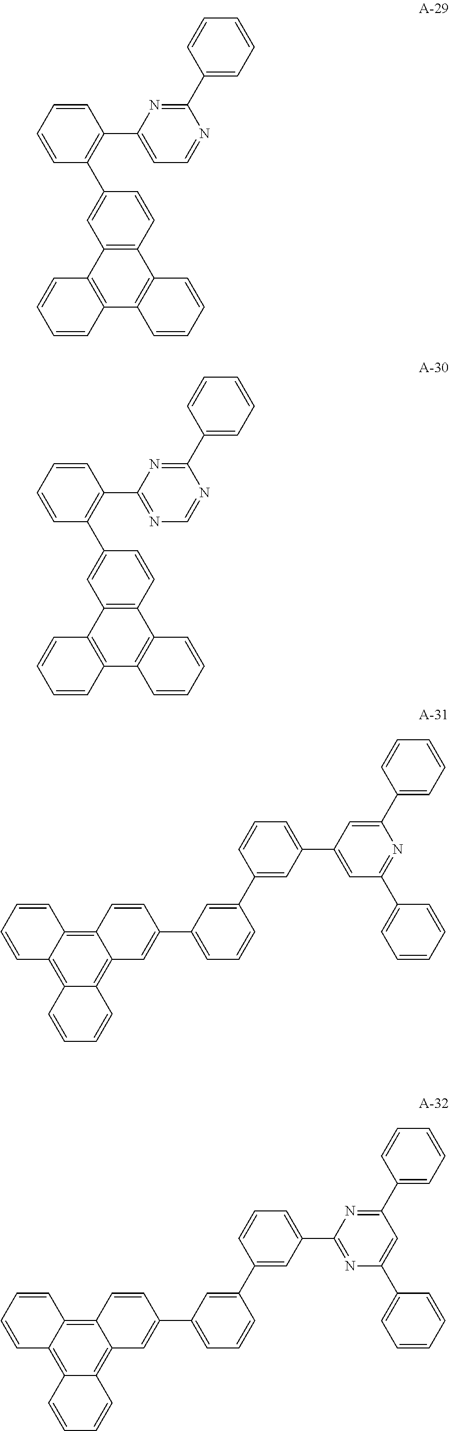 [SMILES: c1ccc(-c2nccc(-c3ccccc3-c3ccc4c5ccccc5c5ccccc5c4c3)n2)cc1 ; c1ccc(-c2ncnc(-c3ccccc3-c3ccc4c5ccccc5c5ccccc5c4c3)n2)cc1 ; c1ccc(-c2cc(-c3cccc(-c4cccc(-c5ccc6c7ccccc7c7ccccc7c6c5)c4)c3)cc(-c3ccccc3)n2)cc1 ; c1ccc(-c2cc(-c3ccccc3)nc(-c3cccc(-c4cccc(-c5ccc6c7ccccc7c7ccccc7c6c5)c4)c3)n2)cc1]

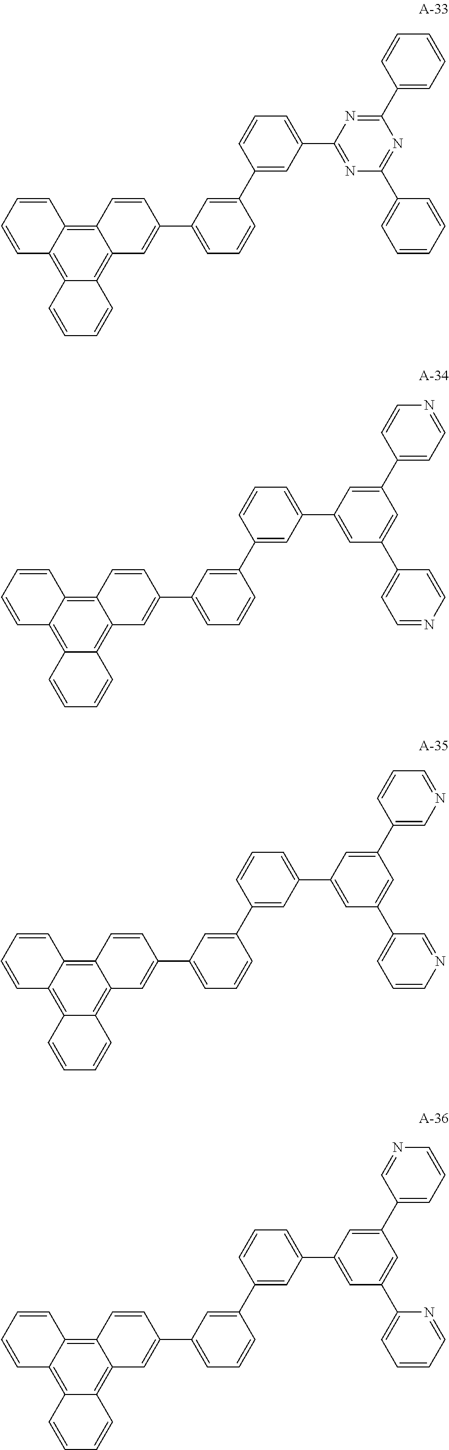 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3ccc4c5ccccc5c5ccccc5c4c3)c2)cc(-c2cc(-c3ccncc3)cc(-c3ccncc3)c2)c1 ; c1cncc(-c2cc(-c3cccnc3)cc(-c3cccc(-c4cccc(-c5ccc6c7ccccc7c7ccccc7c6c5)c4)c3)c2)c1 ; c1ccc(-c2cc(-c3cccnc3)cc(-c3cccc(-c4cccc(-c5ccc6c7ccccc7c7ccccc7c6c5)c4)c3)c2)nc1]

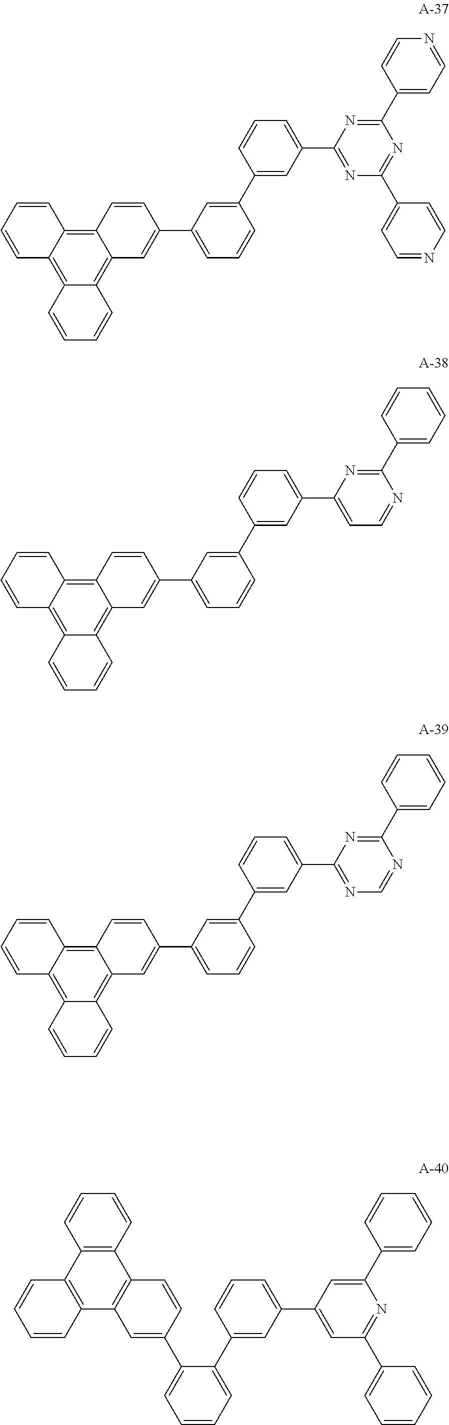 [SMILES: c1cc(-c2cccc(-c3nc(-c4ccncc4)nc(-c4ccncc4)n3)c2)cc(-c2ccc3c4ccccc4c4ccccc4c3c2)c1 ; c1ccc(-c2nccc(-c3cccc(-c4cccc(-c5ccc6c7ccccc7c7ccccc7c6c5)c4)c3)n2)cc1 ; c1ccc(-c2ncnc(-c3cccc(-c4cccc(-c5ccc6c7ccccc7c7ccccc7c6c5)c4)c3)n2)cc1 ; c1ccc(-c2cc(-c3cccc(-c4ccccc4-c4ccc5c6ccccc6c6ccccc6c5c4)c3)cc(-c3ccccc3)n2)cc1]

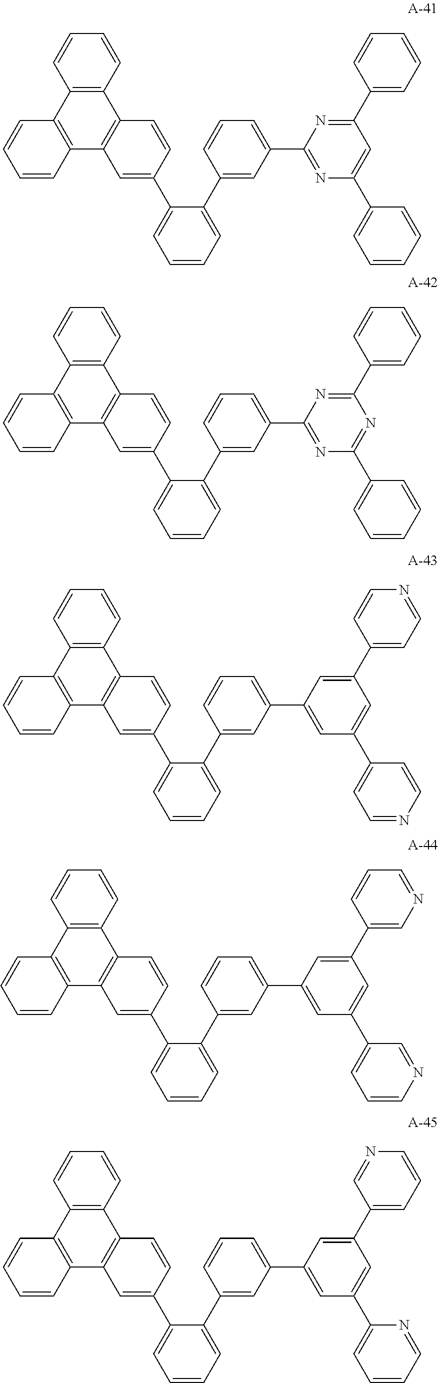

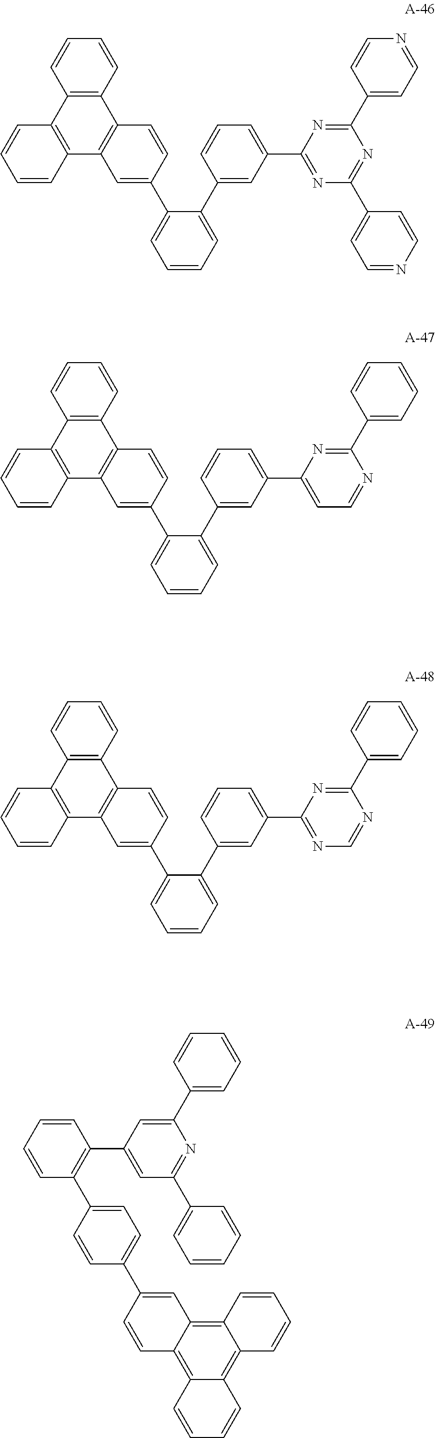 [SMILES: c1cc(-c2nc(-c3ccncc3)nc(-c3ccncc3)n2)cc(-c2ccccc2-c2ccc3c4ccccc4c4ccccc4c3c2)c1 ; c1ccc(-c2nccc(-c3cccc(-c4ccccc4-c4ccc5c6ccccc6c6ccccc6c5c4)c3)n2)cc1 ; c1ccc(-c2ncnc(-c3cccc(-c4ccccc4-c4ccc5c6ccccc6c6ccccc6c5c4)c3)n2)cc1 ; c1ccc(-c2cc(-c3ccccc3-c3ccc(-c4ccc5c6ccccc6c6ccccc6c5c4)cc3)cc(-c3ccccc3)n2)cc1]

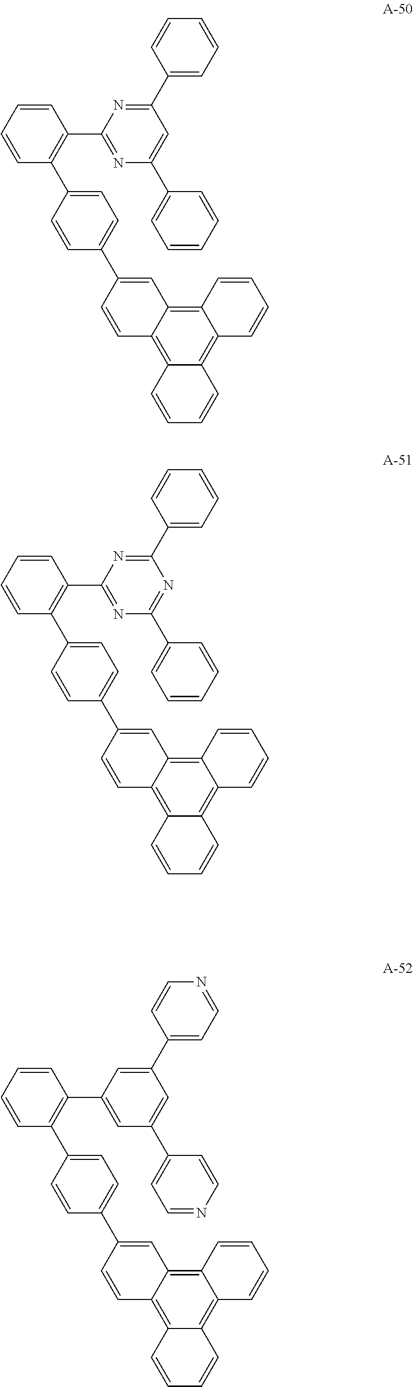 [SMILES: c1ccc(-c2cc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccncc3)cc(-c3ccncc3)c2)c(-c2ccc(-c3ccc4c5ccccc5c5ccccc5c4c3)cc2)c1]

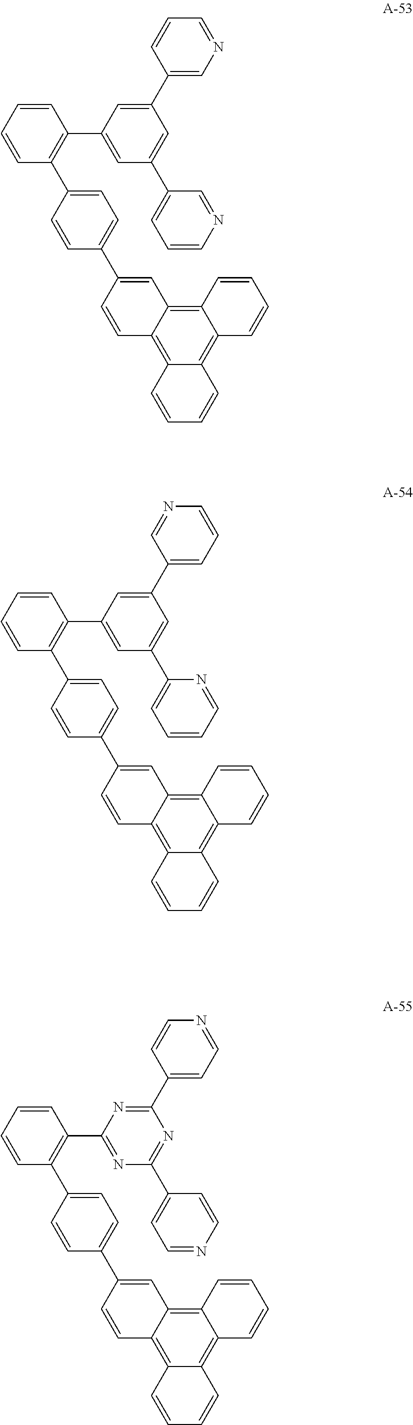 [SMILES: c1cncc(-c2cc(-c3cccnc3)cc(-c3ccccc3-c3ccc(-c4ccc5c6ccccc6c6ccccc6c5c4)cc3)c2)c1 ; c1ccc(-c2cc(-c3cccnc3)cc(-c3ccccc3-c3ccc(-c4ccc5c6ccccc6c6ccccc6c5c4)cc3)c2)nc1 ; c1ccc(-c2nc(-c3ccncc3)nc(-c3ccncc3)n2)c(-c2ccc(-c3ccc4c5ccccc5c5ccccc5c4c3)cc2)c1]

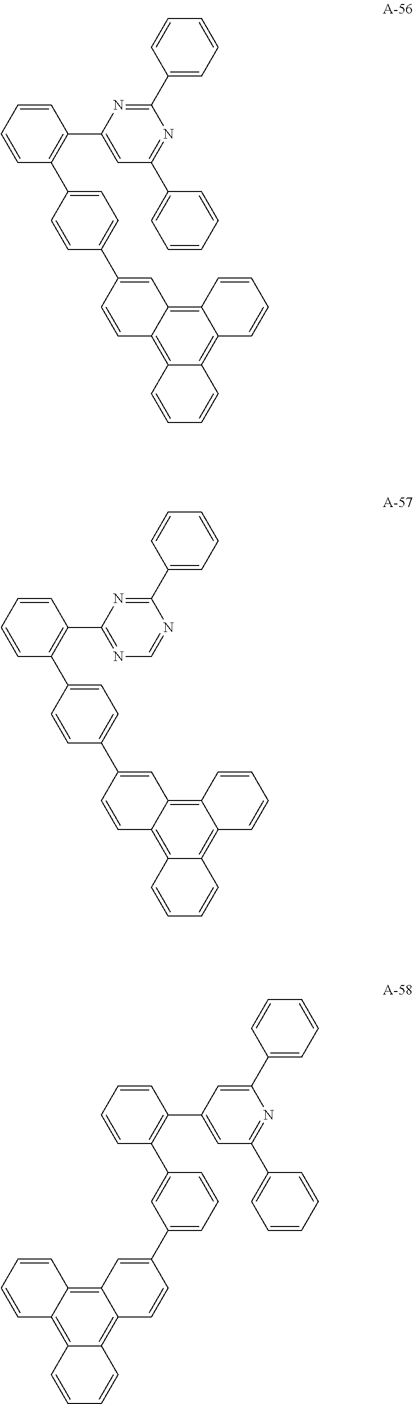 [SMILES: c1ccc(-c2cc(-c3ccccc3-c3ccc(-c4ccc5c6ccccc6c6ccccc6c5c4)cc3)nc(-c3ccccc3)n2)cc1 ; c1ccc(-c2ncnc(-c3ccccc3-c3ccc(-c4ccc5c6ccccc6c6ccccc6c5c4)cc3)n2)cc1 ; c1ccc(-c2cc(-c3ccccc3-c3cccc(-c4ccc5c6ccccc6c6ccccc6c5c4)c3)cc(-c3ccccc3)n2)cc1]

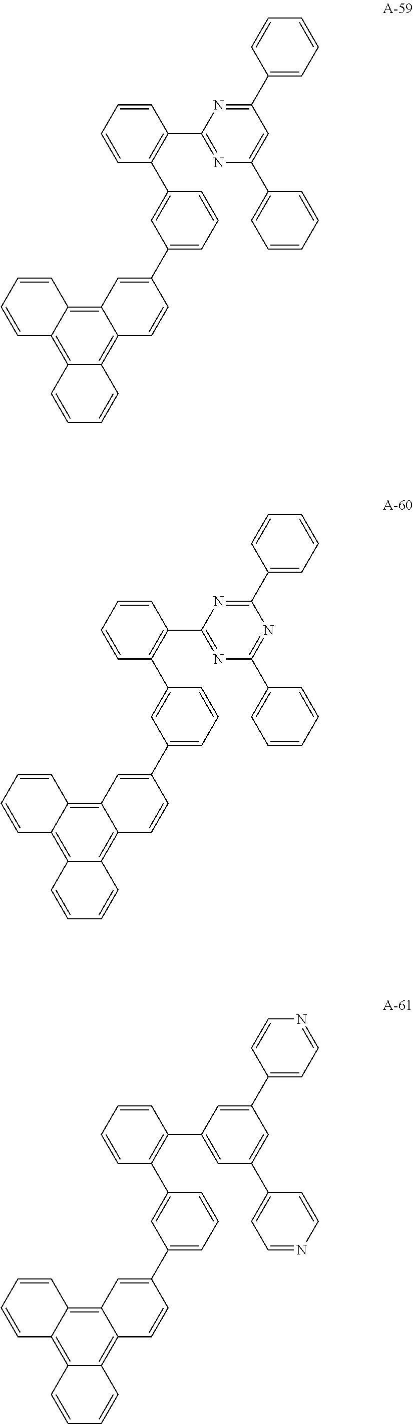 [SMILES: c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1]

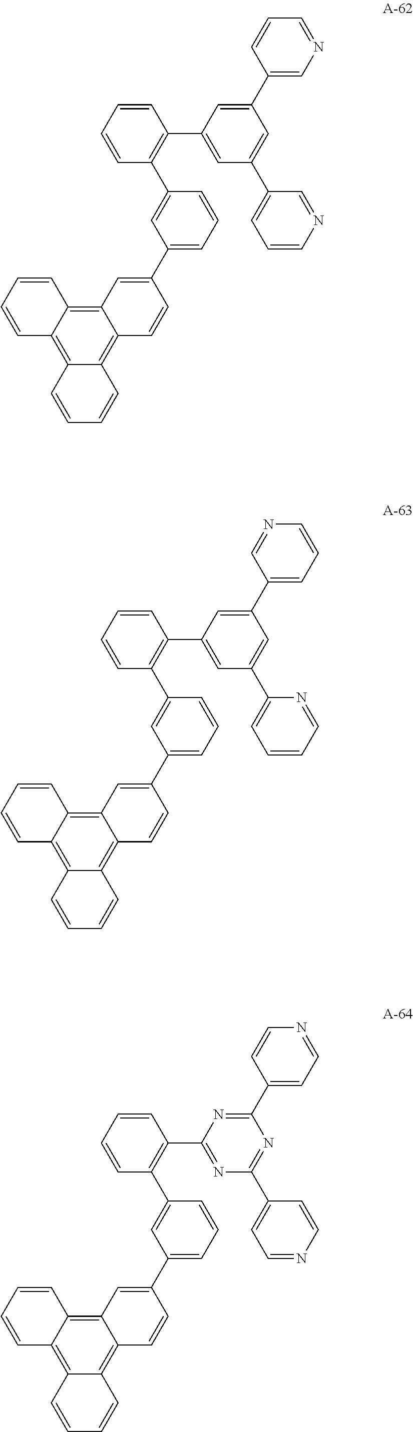 [SMILES: c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1 ; c1cc(-c2ccc3c4ccccc4c4ccccc4c3c2)cc(-c2ccccc2-c2nc(-c3ccncc3)nc(-c3ccncc3)n2)c1]

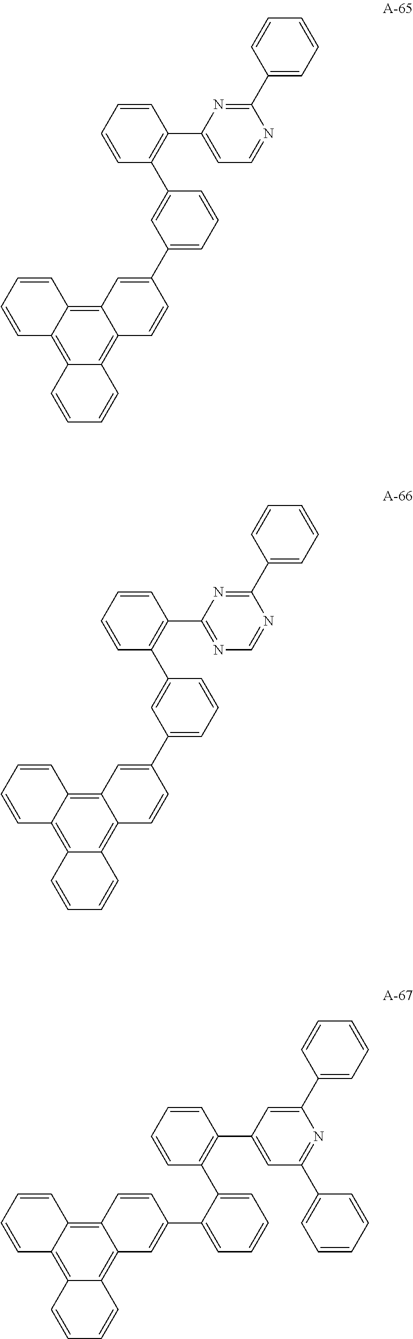 [SMILES: c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1]

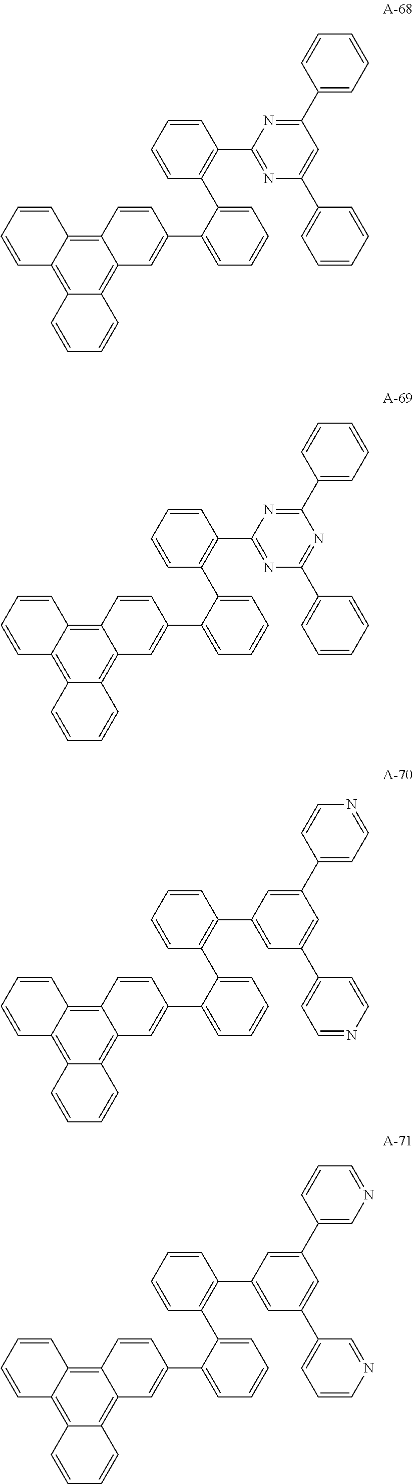 [SMILES: c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccccc3-c3ccc4c5ccccc5c5ccccc5c4c3)c2)c1]

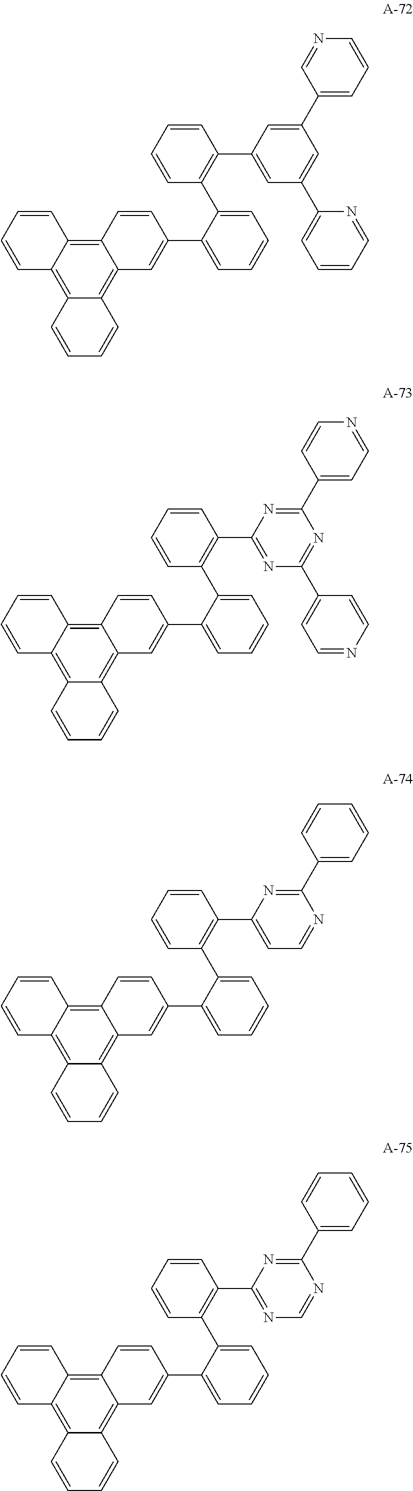 [SMILES: c1ccc(-c2cc(-c3cccnc3)cc(-c3ccccc3-c3ccccc3-c3ccc4c5ccccc5c5ccccc5c4c3)c2)nc1 ; c1ccc(-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccc4c5ccccc5c5ccccc5c4c3)n2)cc1 ; c1ccc(-c2ncnc(-c3ccccc3-c3ccccc3-c3ccc4c5ccccc5c5ccccc5c4c3)n2)cc1]

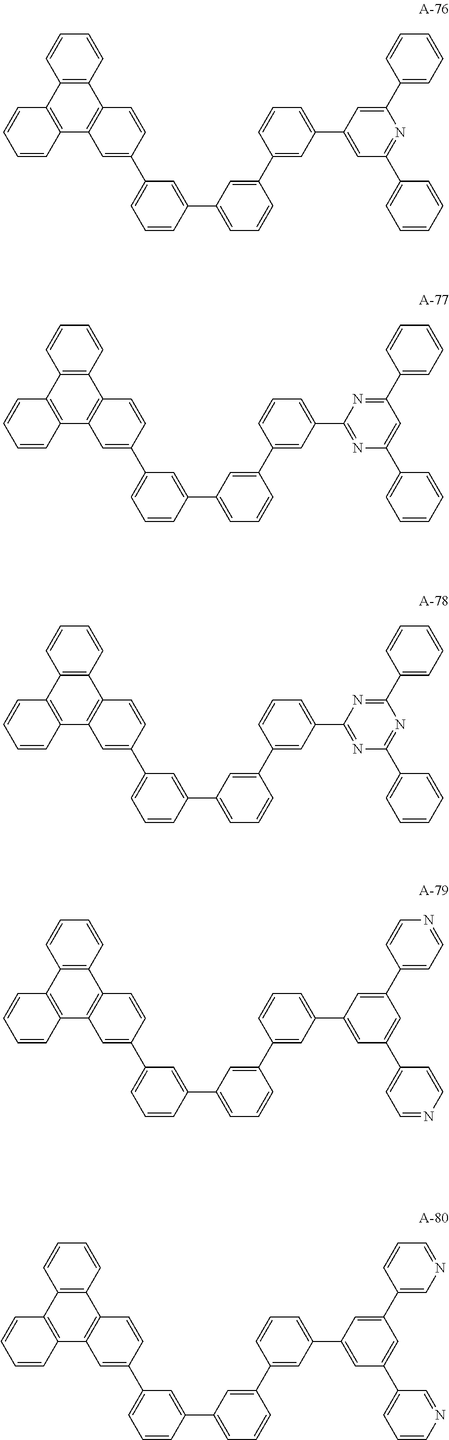 [SMILES: c1ccc(-c2cc(-c3cccc(-c4cccc(-c5cccc(-c6ccc7c8ccccc8c8ccccc8c7c6)c5)c4)c3)cc(-c3ccccc3)n2)cc1 ; c1ccc(-c2cc(-c3ccccc3)nc(-c3cccc(-c4cccc(-c5cccc(-c6ccc7c8ccccc8c8ccccc8c7c6)c5)c4)c3)n2)cc1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1cc(-c2cccc(-c3cc(-c4ccncc4)cc(-c4ccncc4)c3)c2)cc(-c2cccc(-c3ccc4c5ccccc5c5ccccc5c4c3)c2)c1 ; c1cncc(-c2cc(-c3cccnc3)cc(-c3cccc(-c4cccc(-c5cccc(-c6ccc7c8ccccc8c8ccccc8c7c6)c5)c4)c3)c2)c1]

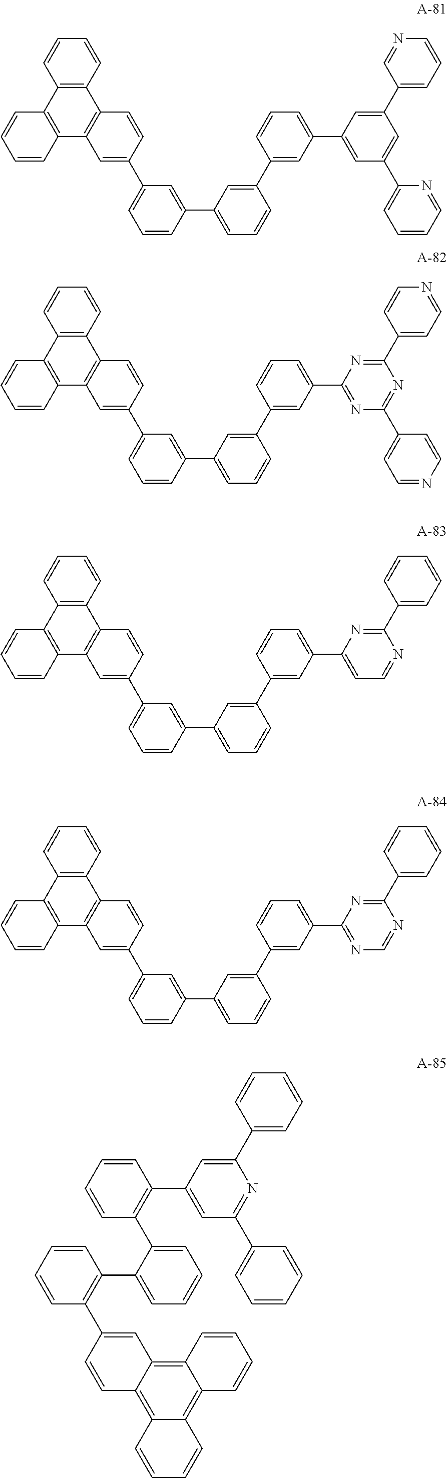 [SMILES: c1ccc(-c2cc(-c3cccnc3)cc(-c3cccc(-c4cccc(-c5cccc(-c6ccc7c8ccccc8c8ccccc8c7c6)c5)c4)c3)c2)nc1 ; c1cc(-c2cccc(-c3ccc4c5ccccc5c5ccccc5c4c3)c2)cc(-c2cccc(-c3nc(-c4ccncc4)nc(-c4ccncc4)n3)c2)c1 ; c1ccc(-c2nccc(-c3cccc(-c4cccc(-c5cccc(-c6ccc7c8ccccc8c8ccccc8c7c6)c5)c4)c3)n2)cc1 ; c1ccc(-c2ncnc(-c3cccc(-c4cccc(-c5cccc(-c6ccc7c8ccccc8c8ccccc8c7c6)c5)c4)c3)n2)cc1 ; c1ccc(-c2cc(-c3ccccc3-c3ccccc3-c3ccccc3-c3ccc4c5ccccc5c5ccccc5c4c3)cc(-c3ccccc3)n2)cc1]

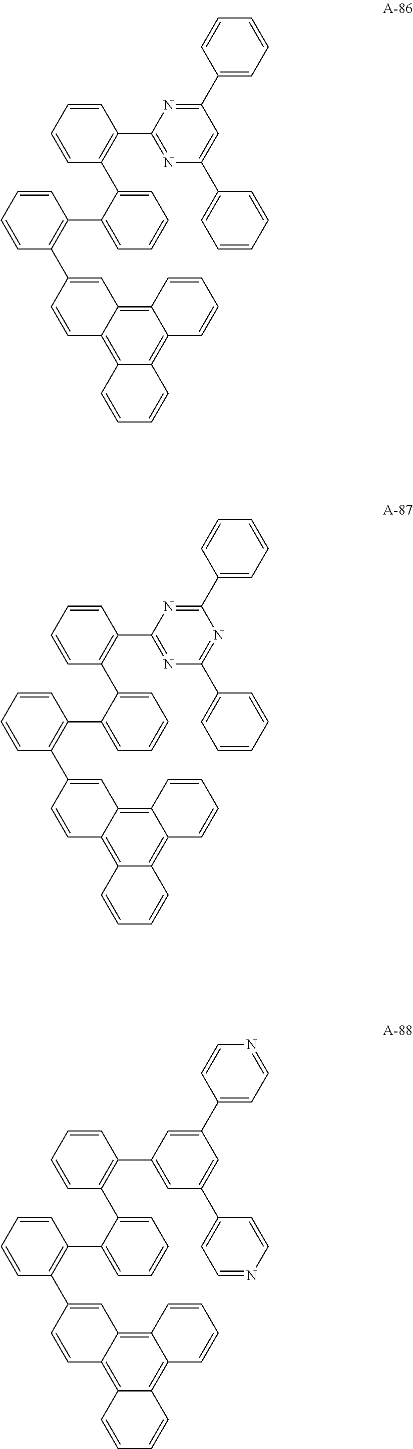 [SMILES: c1ccc(-c2cc(-c3ccccc3)nc(-c3ccccc3-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2ccc3c4ccccc4c4ccccc4c3c2)c(-c2cc(-c3ccncc3)cc(-c3ccncc3)c2)c1]

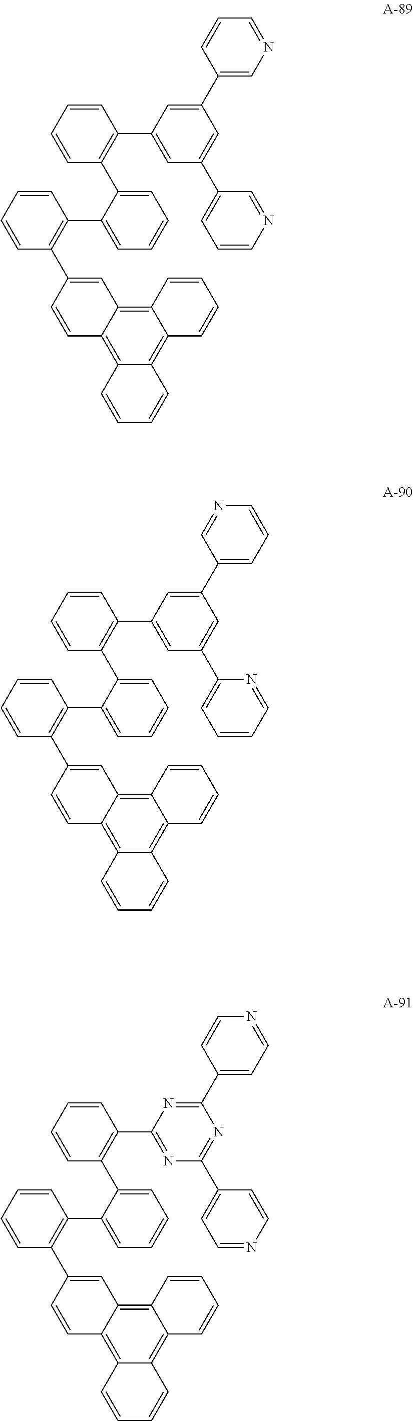 [SMILES: c1cncc(-c2cc(-c3cccnc3)cc(-c3ccccc3-c3ccccc3-c3ccccc3-c3ccc4c5ccccc5c5ccccc5c4c3)c2)c1 ; c1ccc(-c2cc(-c3cccnc3)cc(-c3ccccc3-c3ccccc3-c3ccccc3-c3ccc4c5ccccc5c5ccccc5c4c3)c2)nc1 ; c1ccc(-c2ccccc2-c2ccccc2-c2nc(-c3ccncc3)nc(-c3ccncc3)n2)c(-c2ccc3c4ccccc4c4ccccc4c3c2)c1]

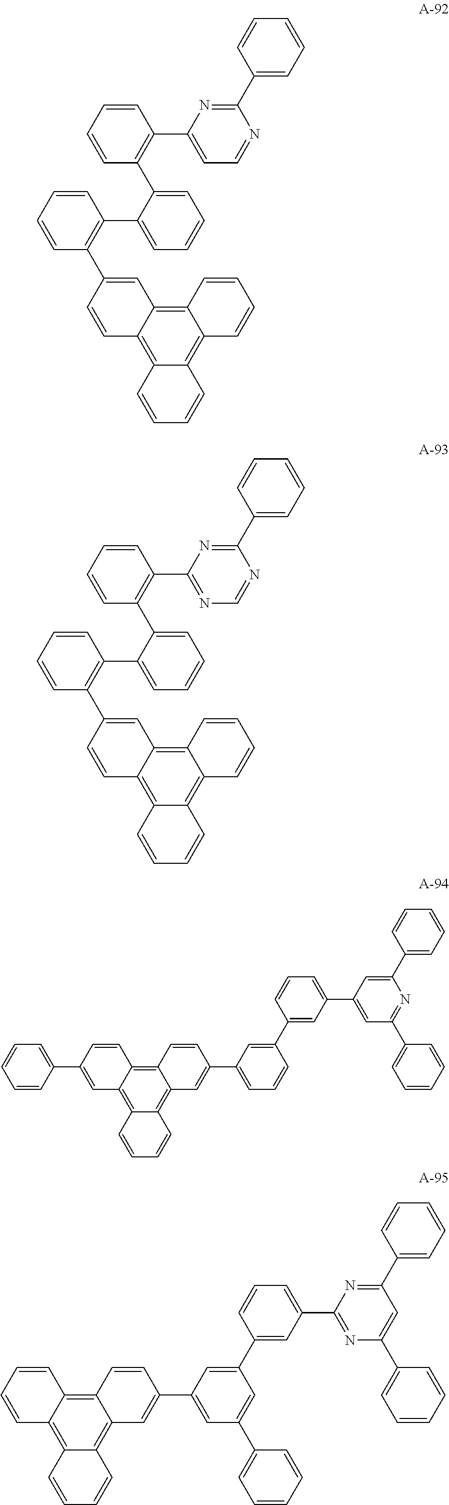 [SMILES: c1ccc(-c2nccc(-c3ccccc3-c3ccccc3-c3ccccc3-c3ccc4c5ccccc5c5ccccc5c4c3)n2)cc1 ; c1ccc(-c2ncnc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1]

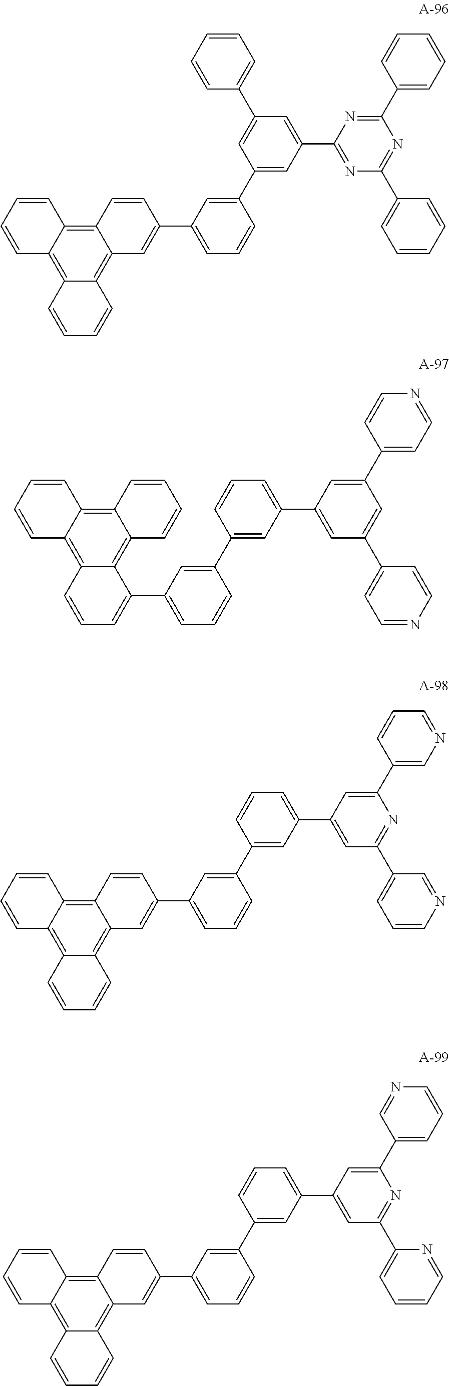 [SMILES: c1ccc(-c2cc(-c3cccc(-c4ccc5c6ccccc6c6ccccc6c5c4)c3)cc(-c3nc(-c4ccccc4)nc(-c4ccccc4)n3)c2)cc1 ; c1cc(-c2cc(-c3ccncc3)cc(-c3ccncc3)c2)cc(-c2cccc(-c3cccc4c5ccccc5c5ccccc5c34)c2)c1 ; c1cc(-c2cccc(-c3ccc4c5ccccc5c5ccccc5c4c3)c2)cc(-c2cc(-c3cccnc3)nc(-c3cccnc3)c2)c1 ; c1ccc(-c2cc(-c3cccc(-c4cccc(-c5ccc6c7ccccc7c7ccccc7c6c5)c4)c3)cc(-c3cccnc3)n2)nc1]

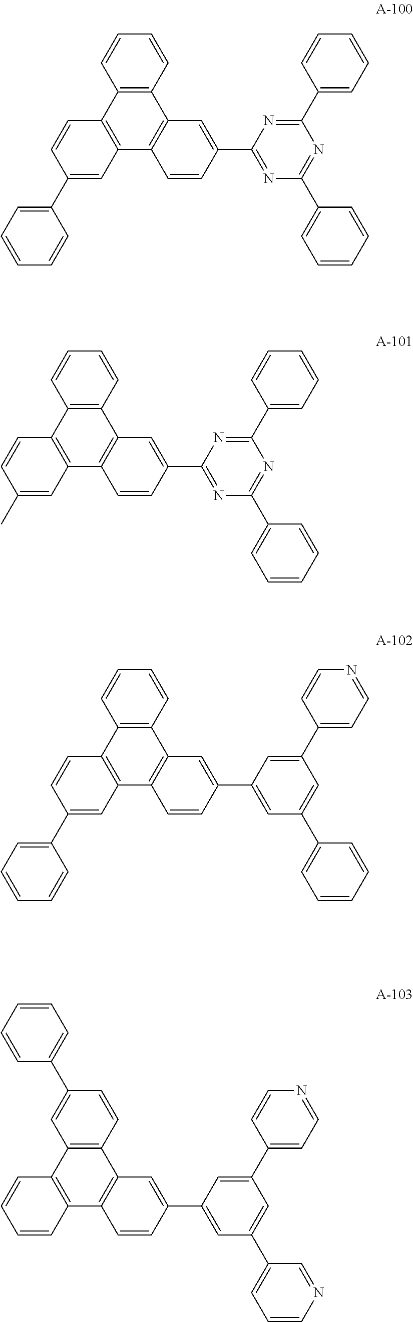 [SMILES: c1ccc(-c2ccc3c4ccccc4c4cc(-c5nc(-c6ccccc6)nc(-c6ccccc6)n5)ccc4c3c2)cc1 ; Cc1ccc2c3ccccc3c3cc(-c4nc(-c5ccccc5)nc(-c5ccccc5)n4)ccc3c2c1 ; c1ccc(-c2cc(-c3ccncc3)cc(-c3ccc4c5cc(-c6ccccc6)ccc5c5ccccc5c4c3)c2)cc1 ; c1ccc(-c2ccc3c(c2)c2ccccc2c2ccc(-c4cc(-c5ccncc5)cc(-c5cccnc5)c4)cc23)cc1]

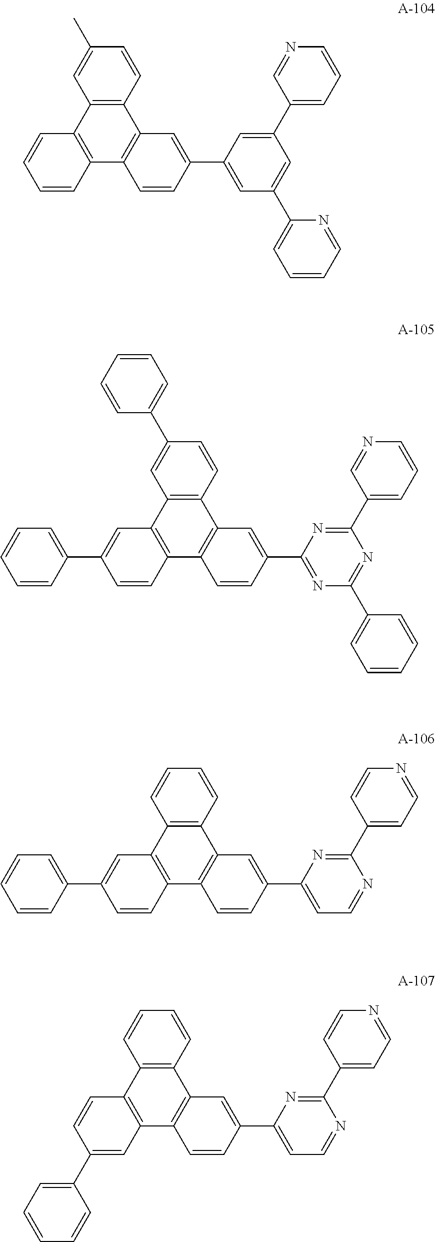 [SMILES: Cc1ccc2c(c1)c1ccccc1c1ccc(-c3cc(-c4cccnc4)cc(-c4ccccn4)c3)cc12 ; c1ccc(-c2ccc3c4ccc(-c5nc(-c6ccccc6)nc(-c6cccnc6)n5)cc4c4ccc(-c5ccccc5)cc4c3c2)cc1 ; c1ccc(-c2ccc3c4ccc(-c5ccnc(-c6ccncc6)n5)cc4c4ccccc4c3c2)cc1 ; c1ccc(-c2ccc3c4ccccc4c4cc(-c5ccnc(-c6ccncc6)n5)ccc4c3c2)cc1]

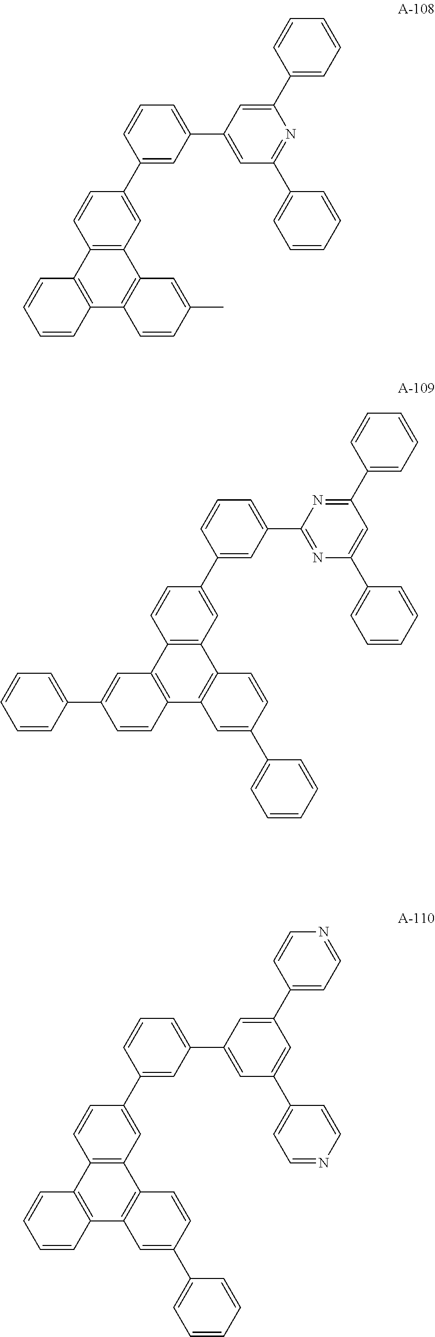 [SMILES: Cc1ccc2c3ccccc3c3ccc(-c4cccc(-c5cc(-c6ccccc6)nc(-c6ccccc6)c5)c4)cc3c2c1 ; c1ccc(-c2ccc3c(c2)c2ccc(-c4cccc(-c5nc(-c6ccccc6)cc(-c6ccccc6)n5)c4)cc2c2ccc(-c4ccccc4)cc32)cc1 ; c1ccc(-c2ccc3c(c2)c2ccccc2c2ccc(-c4cccc(-c5cc(-c6ccncc6)cc(-c6ccncc6)c5)c4)cc23)cc1]

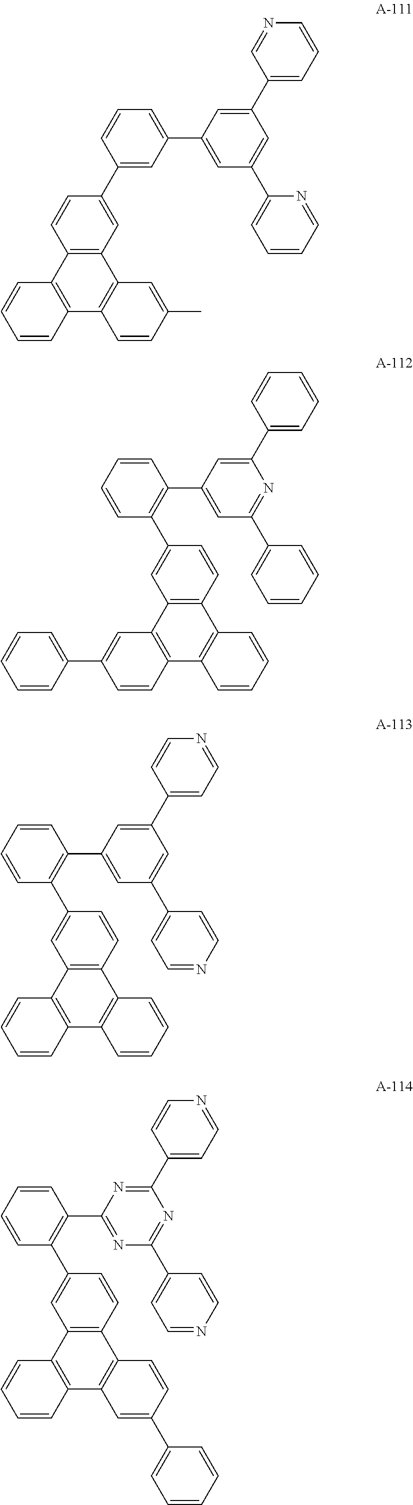 [SMILES: Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1 ; c1ccc(-c2ccc3c4ccccc4c4ccccc4c3c2)c(-c2cc(-c3ccncc3)cc(-c3ccncc3)c2)c1 ; c1ccc(-c2ccc3c4ccc(-c5ccccc5-c5nc(-c6ccncc6)nc(-c6ccncc6)n5)cc4c4ccccc4c3c2)cc1]

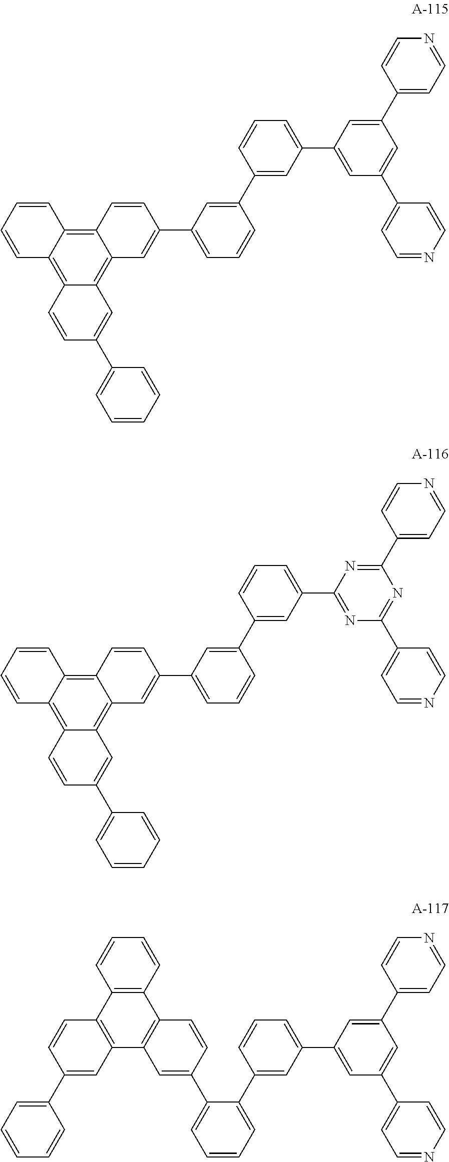 [SMILES: c1ccc(-c2ccc3c4ccccc4c4ccc(-c5cccc(-c6cccc(-c7cc(-c8ccncc8)cc(-c8ccncc8)c7)c6)c5)cc4c3c2)cc1 ; c1ccc(-c2ccc3c4ccccc4c4ccc(-c5cccc(-c6cccc(-c7nc(-c8ccncc8)nc(-c8ccncc8)n7)c6)c5)cc4c3c2)cc1 ; c1ccc(-c2ccc3c4ccccc4c4ccc(-c5ccccc5-c5cccc(-c6cc(-c7ccncc7)cc(-c7ccncc7)c6)c5)cc4c3c2)cc1]

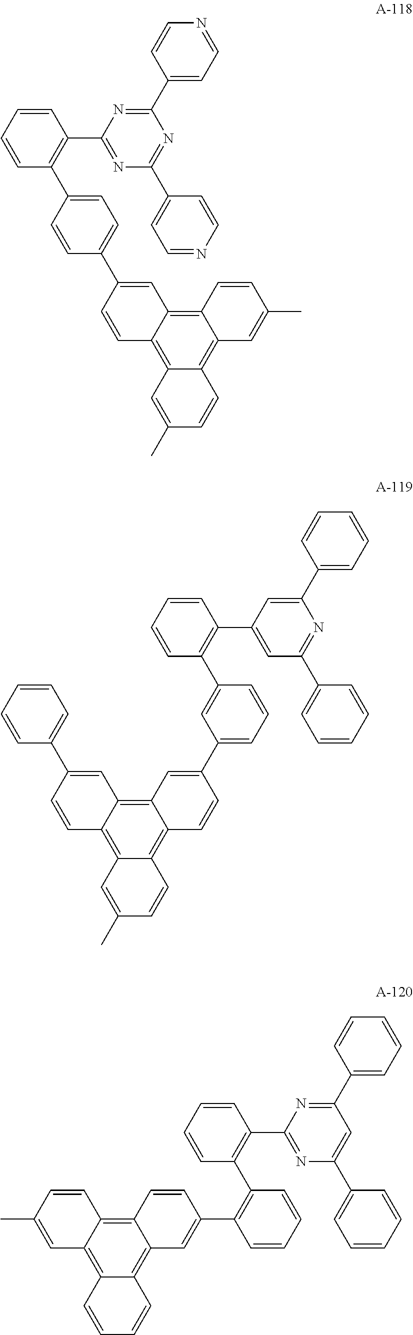

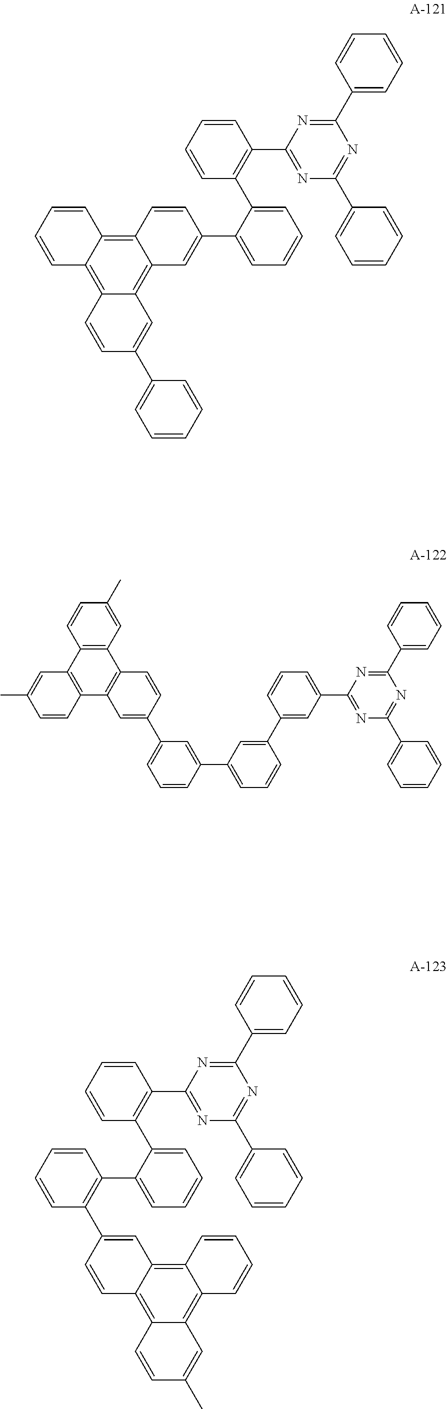 [SMILES: c1ccc(-c2ccc3c4ccccc4c4ccc(-c5ccccc5-c5ccccc5-c5nc(-c6ccccc6)nc(-c6ccccc6)n5)cc4c3c2)cc1 ; Cc1ccc2c(c1)c1ccc(C)cc1c1ccc(-c3cccc(-c4cccc(-c5cccc(-c6nc(-c7ccccc7)nc(-c7ccccc7)n6)c5)c4)c3)cc21 ; Cc1ccc2c3ccc(-c4ccccc4-c4ccccc4-c4ccccc4-c4nc(-c5ccccc5)nc(-c5ccccc5)n4)cc3c3ccccc3c2c1]

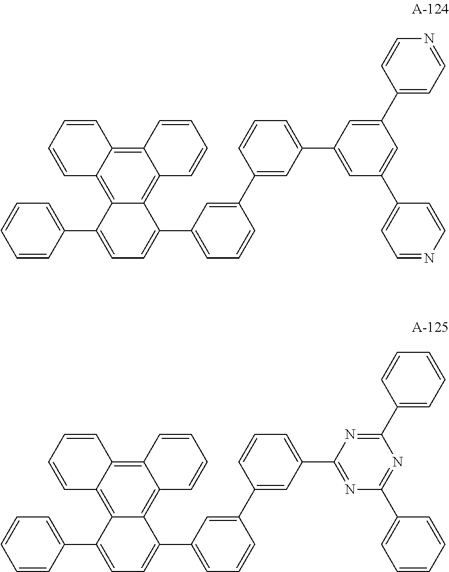 [SMILES: c1ccc(-c2ccc(-c3cccc(-c4cccc(-c5cc(-c6ccncc6)cc(-c6ccncc6)c5)c4)c3)c3c4ccccc4c4ccccc4c23)cc1 ; c1ccc(-c2nc(-c3ccccc3)nc(-c3cccc(-c4cccc(-c5ccc(-c6ccccc6)c6c7ccccc7c7ccccc7c56)c4)c3)n2)cc1]

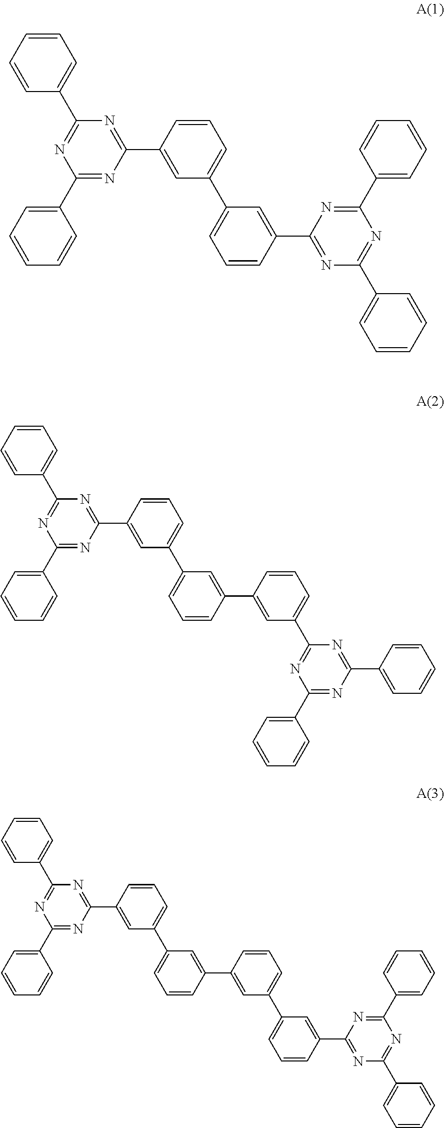 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8)nc(-c8ccccc8)n7)c6)c5)c4)c3)n2)cc1]

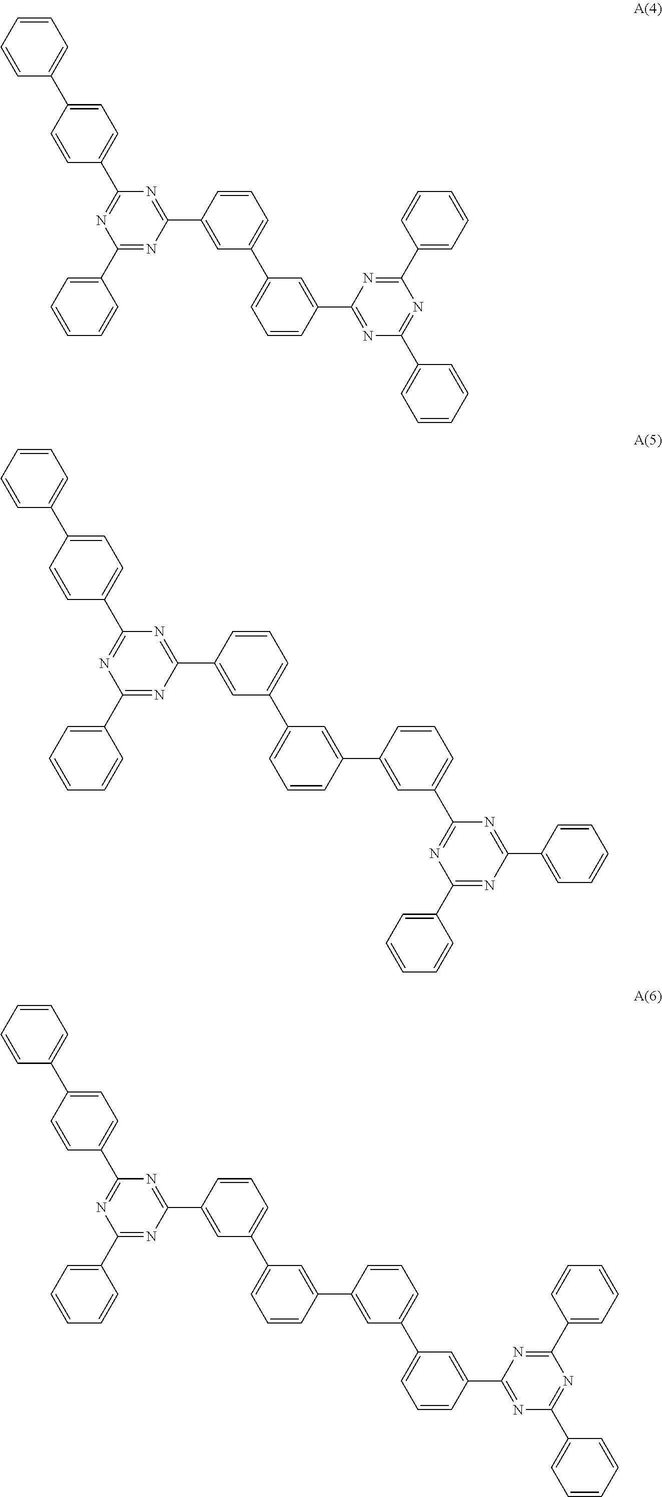 [SMILES: c1ccc(-c2ccc(-c3nc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

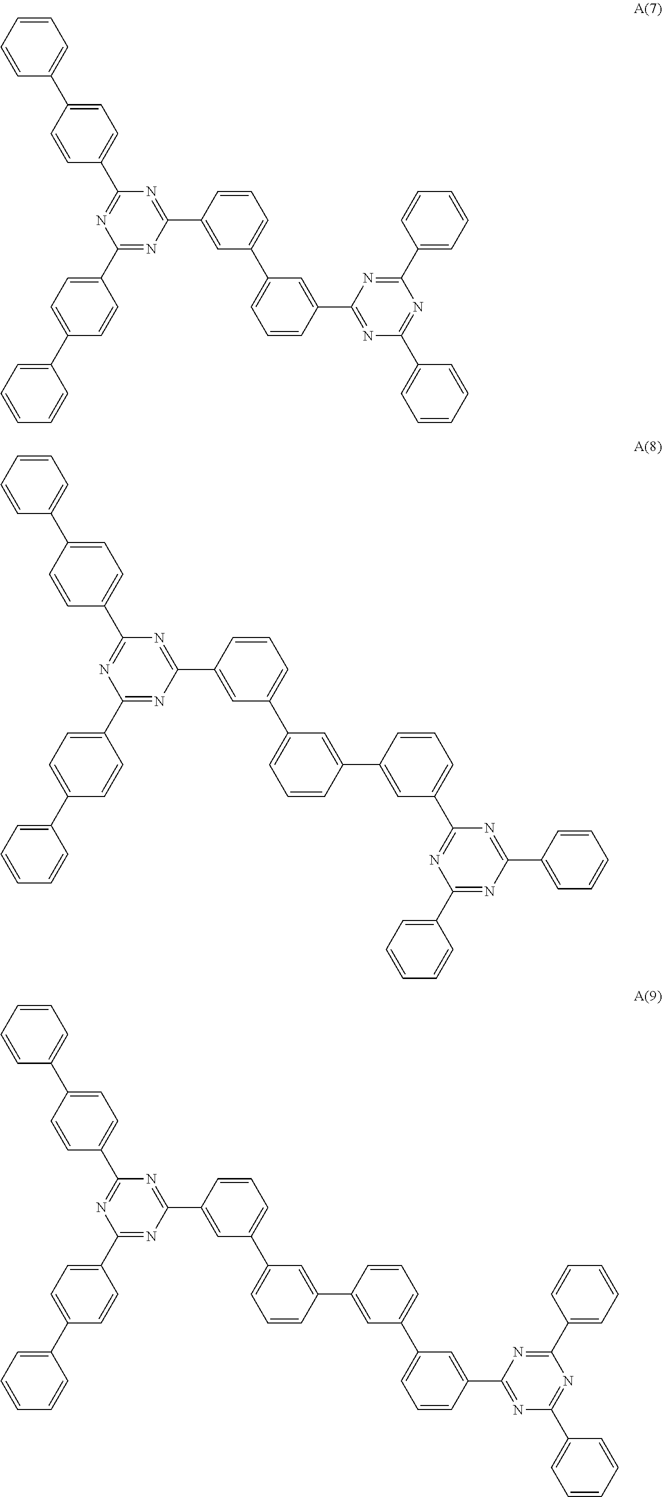

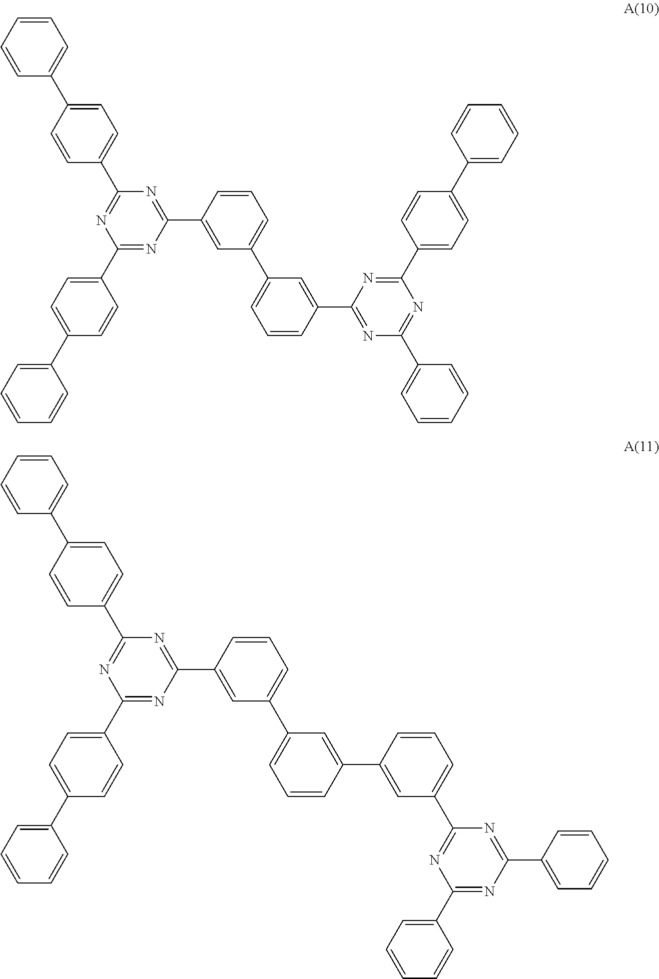

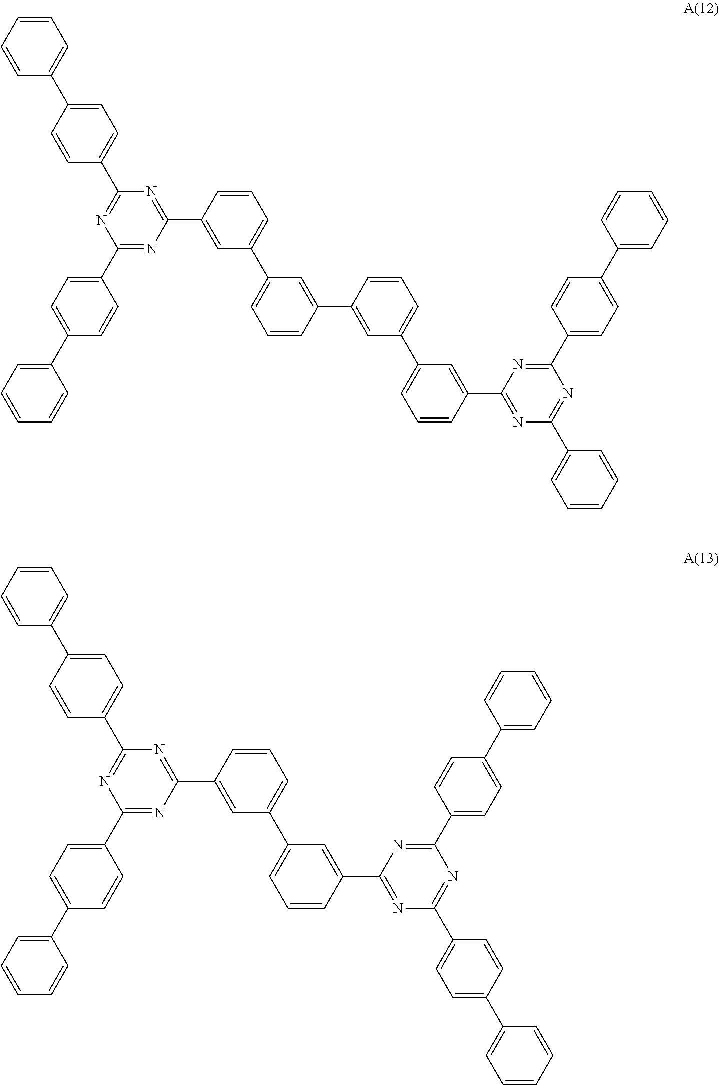 [SMILES: c1ccc(-c2ccc(-c3nc(-c4ccccc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1 ; c1ccc(-c2ccc(-c3nc(-c4ccc(-c5ccccc5)cc4)nc(-c4cccc(-c5cccc(-c6nc(-c7ccc(-c8ccccc8)cc7)nc(-c7ccc(-c8ccccc8)cc7)n6)c5)c4)n3)cc2)cc1]

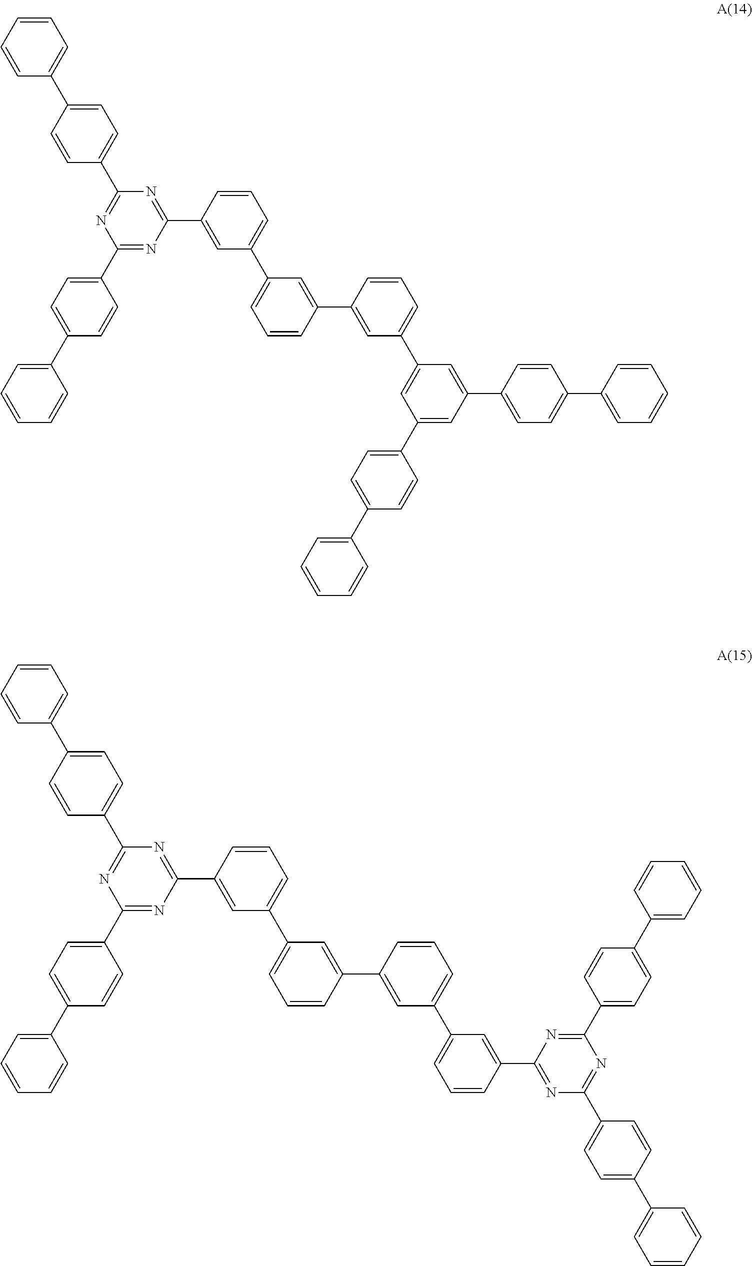 [SMILES: c1ccc(-c2ccc(-c3cc(-c4ccc(-c5ccccc5)cc4)cc(-c4cccc(-c5cccc(-c6cccc(-c7nc(-c8ccc(-c9ccccc9)cc8)nc(-c8ccc(-c9ccccc9)cc8)n7)c6)c5)c4)c3)cc2)cc1 ; c1ccc(-c2ccc(-c3nc(-c4ccc(-c5ccccc5)cc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

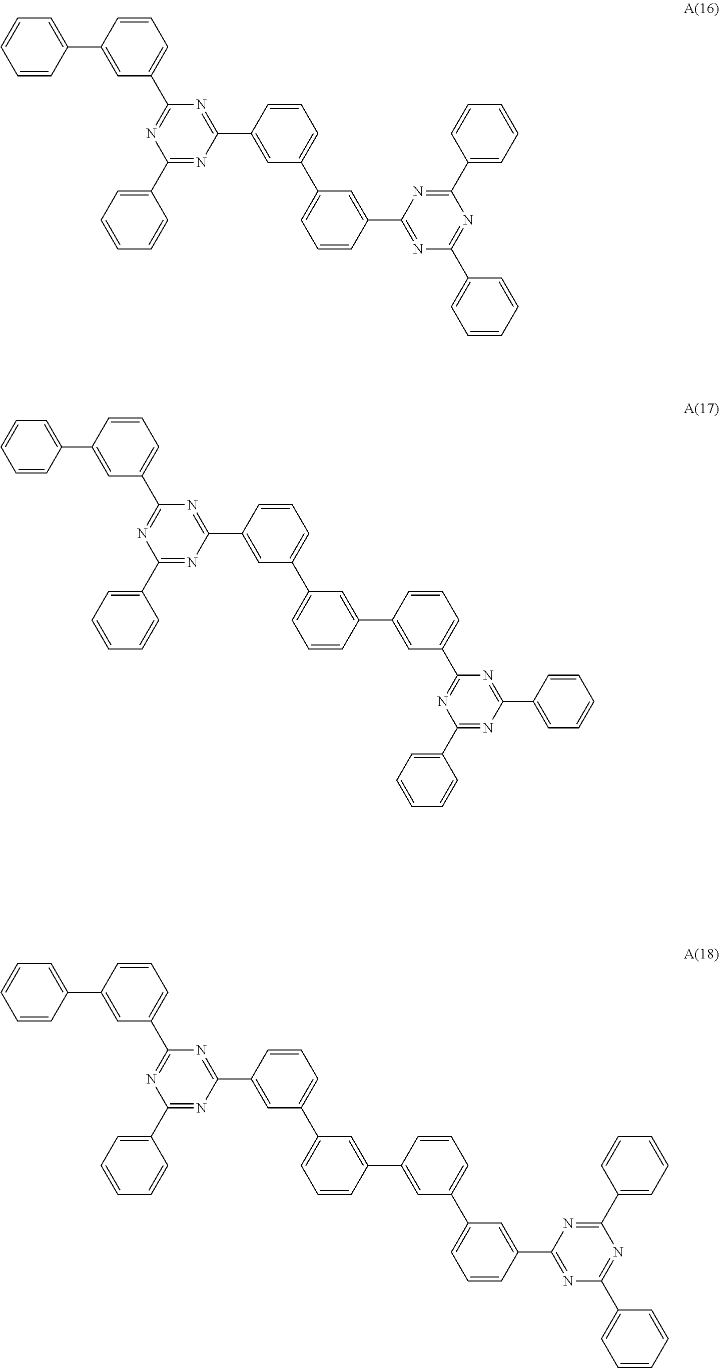 [SMILES: c1ccc(-c2cccc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

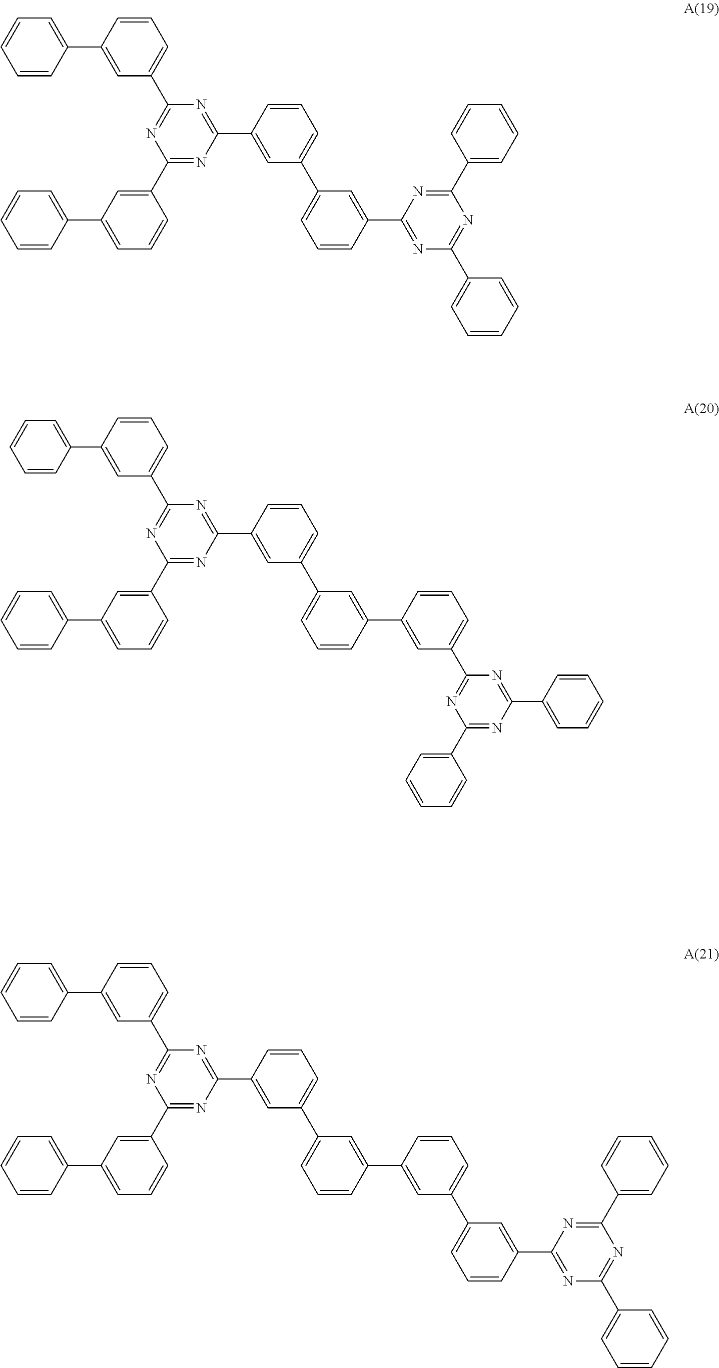 [SMILES: c1ccc(-c2cccc(-c3nc(-c4cccc(-c5ccccc5)c4)nc(-c4cccc(-c5cccc(-c6nc(-c7ccccc7)nc(-c7ccccc7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)c2)cc1]

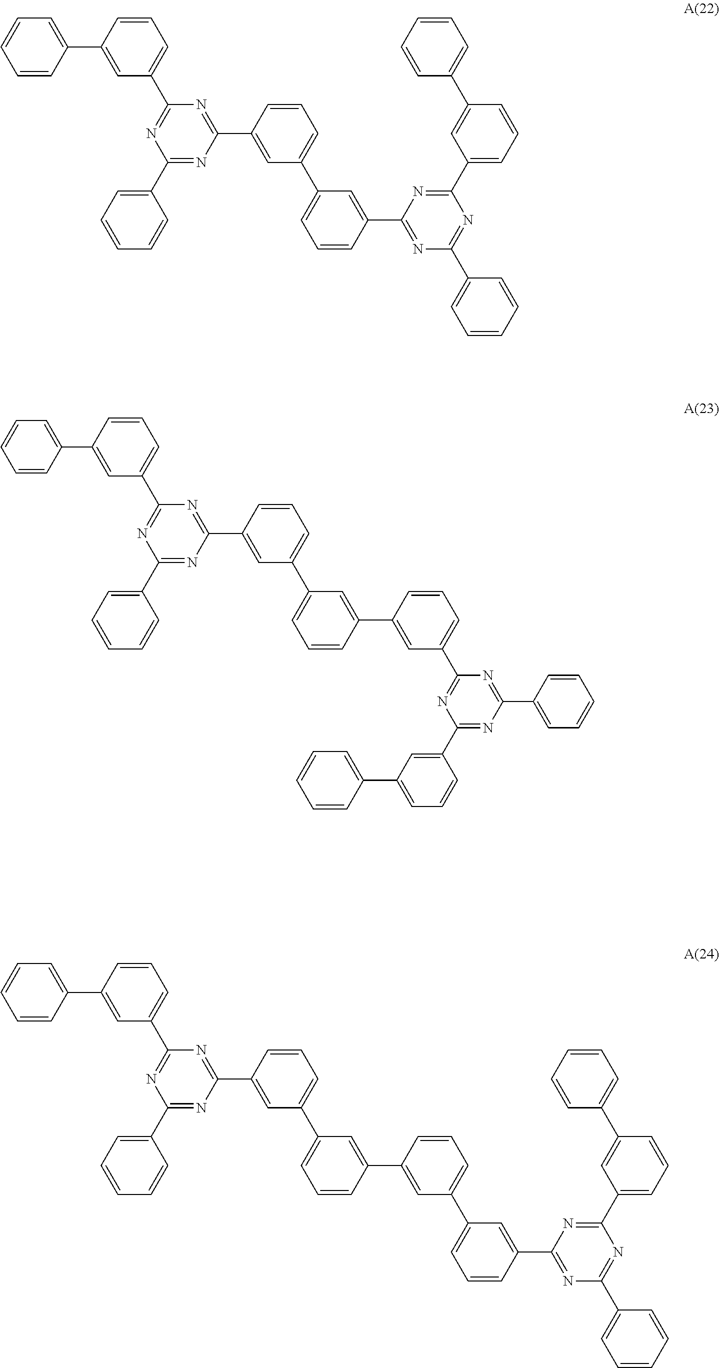 [SMILES: c1ccc(-c2cccc(-c3nc(-c4ccccc4)nc(-c4cccc(-c5cccc(-c6nc(-c7ccccc7)nc(-c7cccc(-c8ccccc8)c7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)c2)cc1]

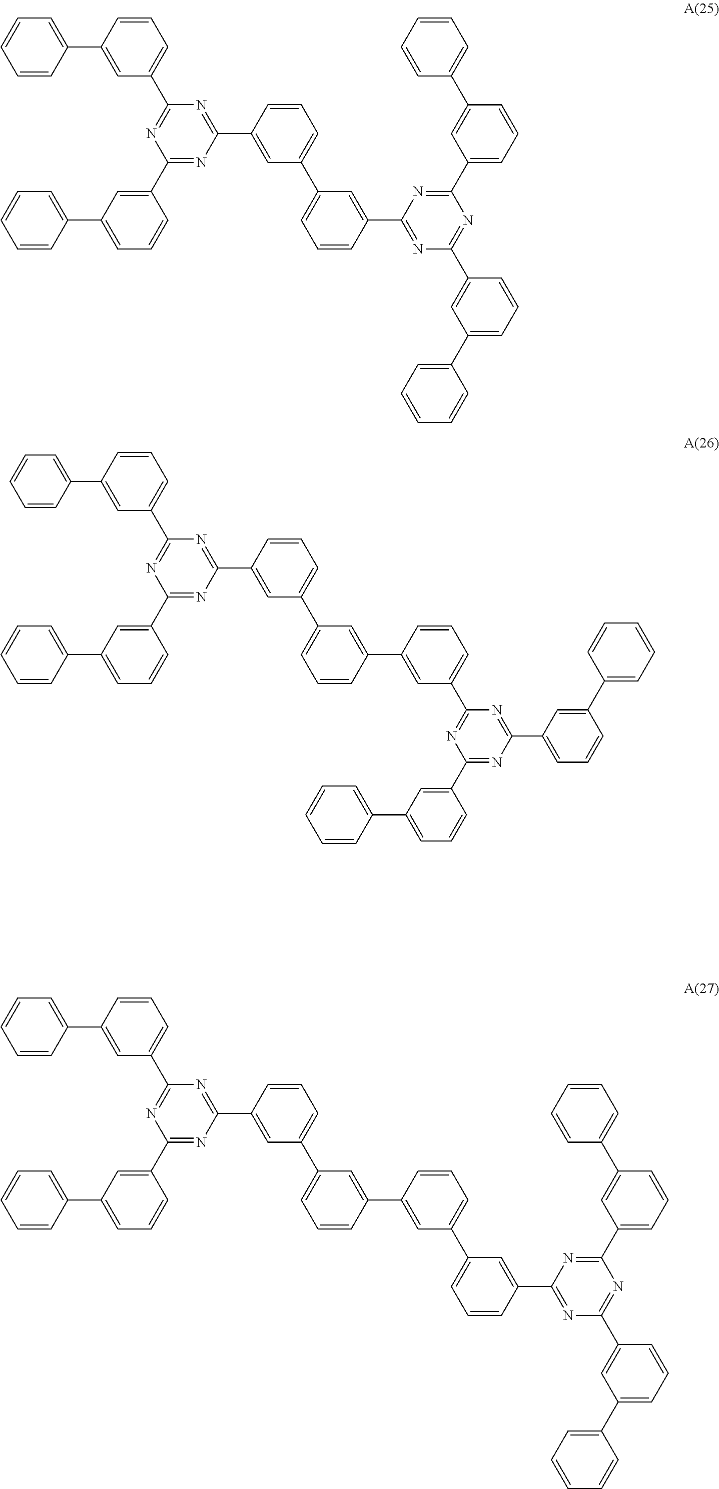 [SMILES: c1ccc(-c2cccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)c2)cc1]

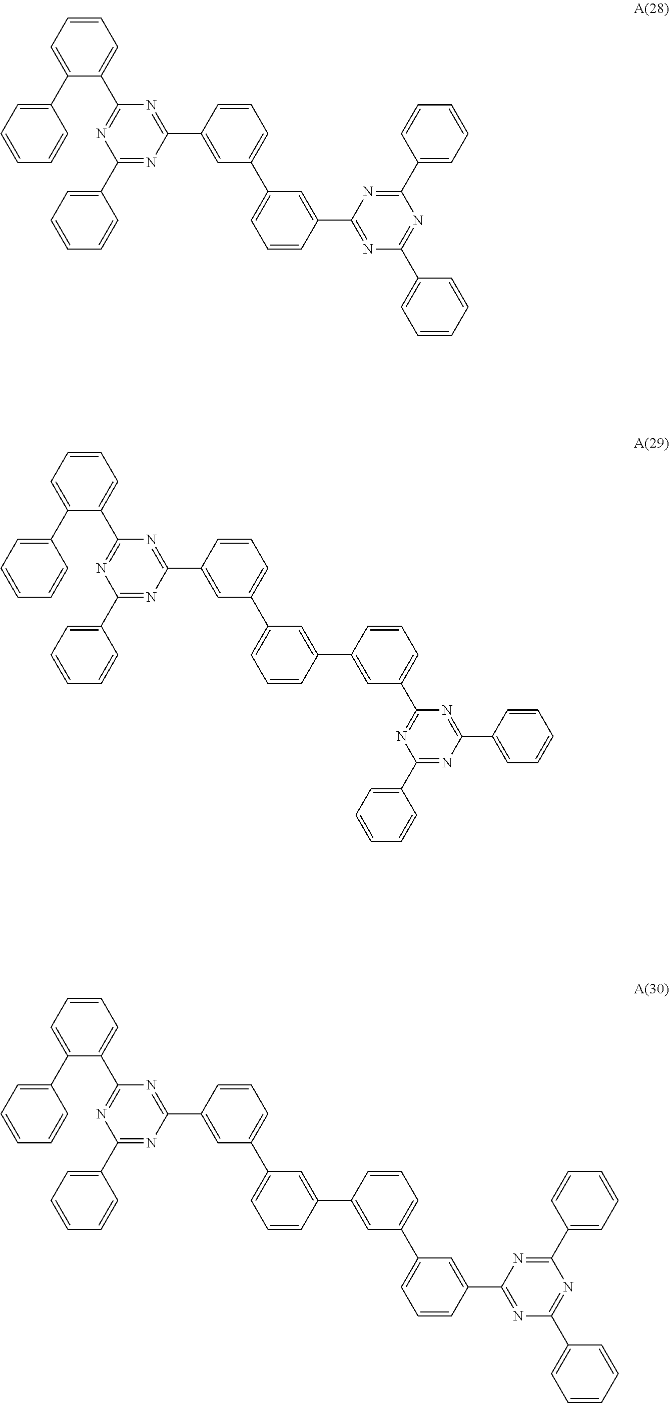 [SMILES: c1ccc(-c2nc(-c3ccccc3)nc(-c3cccc(-c4cccc(-c5nc(-c6ccccc6)nc(-c6ccccc6-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8)nc(-c8ccccc8-c8ccccc8)n7)c6)c5)c4)c3)n2)cc1]

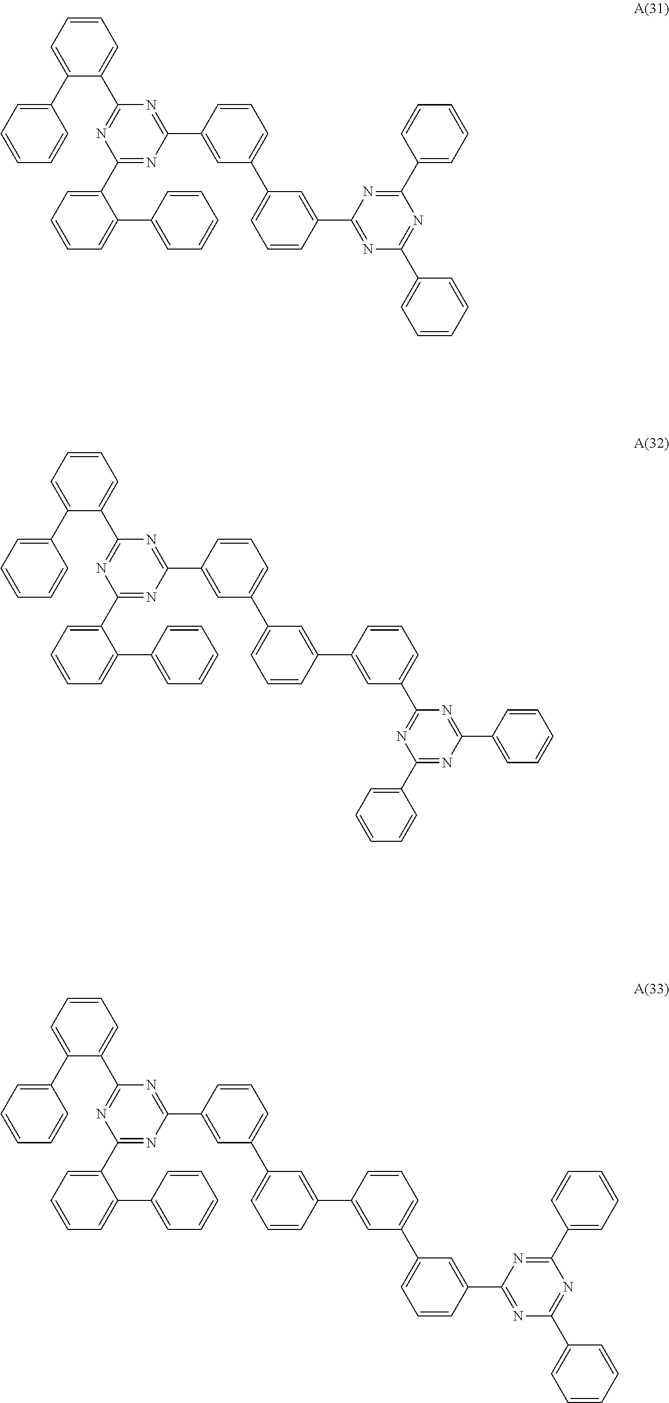 [SMILES: c1ccc(-c2nc(-c3ccccc3)nc(-c3cccc(-c4cccc(-c5nc(-c6ccccc6-c6ccccc6)nc(-c6ccccc6-c6ccccc6)n5)c4)c3)n2)cc1 ; c1ccc(-c2nc(-c3ccccc3)nc(-c3cccc(-c4cccc(-c5cccc(-c6nc(-c7ccccc7-c7ccccc7)nc(-c7ccccc7-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8-c8ccccc8)nc(-c8ccccc8-c8ccccc8)n7)c6)c5)c4)c3)n2)cc1]

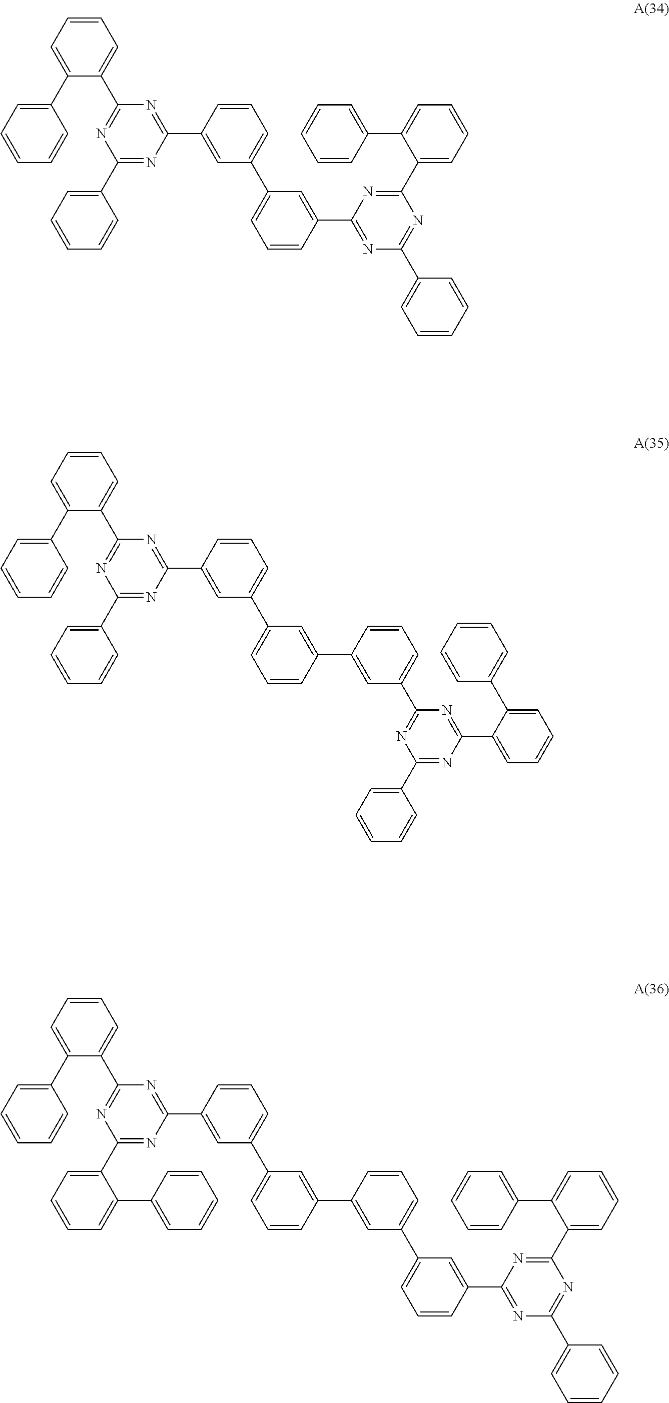 [SMILES: c1ccc(-c2nc(-c3cccc(-c4cccc(-c5nc(-c6ccccc6)nc(-c6ccccc6-c6ccccc6)n5)c4)c3)nc(-c3ccccc3-c3ccccc3)n2)cc1 ; c1ccc(-c2nc(-c3cccc(-c4cccc(-c5cccc(-c6nc(-c7ccccc7)nc(-c7ccccc7-c7ccccc7)n6)c5)c4)c3)nc(-c3ccccc3-c3ccccc3)n2)cc1 ; c1ccc(-c2nc(-c3cccc(-c4cccc(-c5cccc(-c6cccc(-c7nc(-c8ccccc8-c8ccccc8)nc(-c8ccccc8-c8ccccc8)n7)c6)c5)c4)c3)nc(-c3ccccc3-c3ccccc3)n2)cc1]

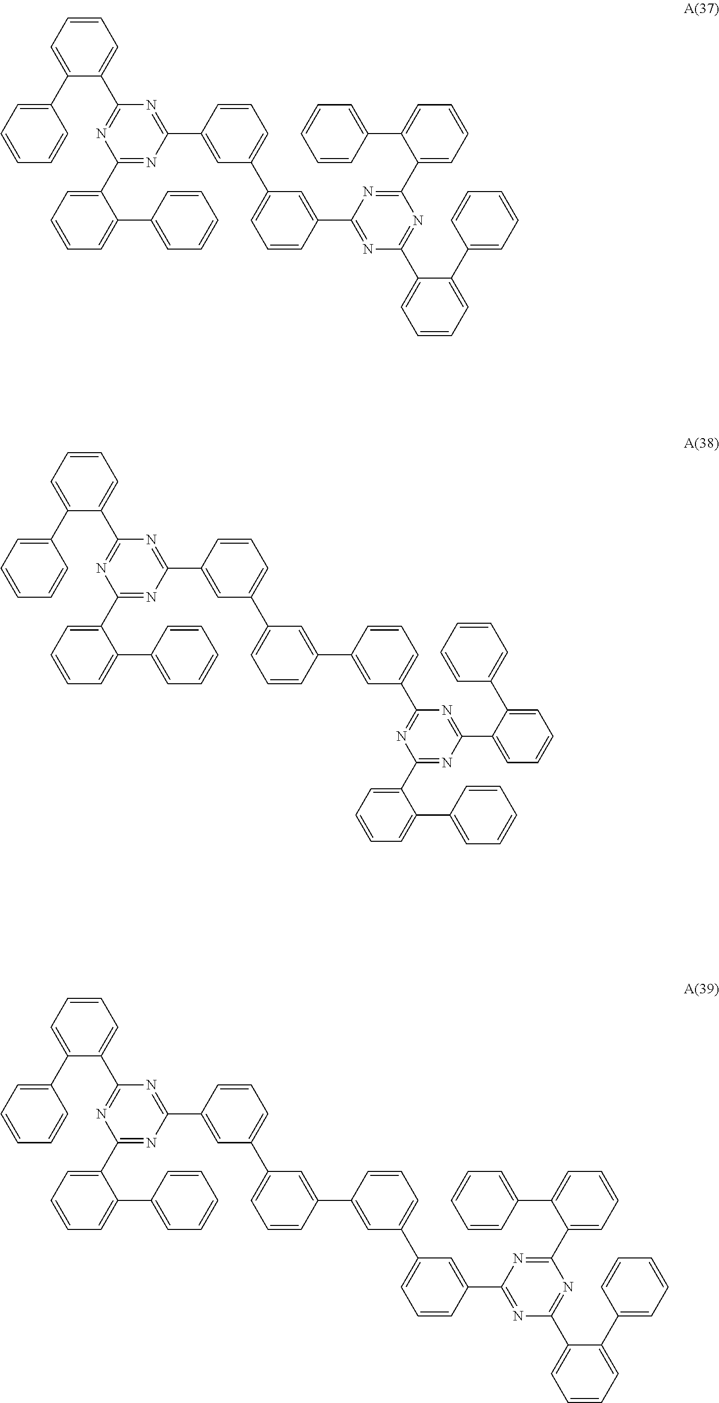 [SMILES: c1ccc(-c2ccccc2-c2nc(-c3cccc(-c4cccc(-c5nc(-c6ccccc6-c6ccccc6)nc(-c6ccccc6-c6ccccc6)n5)c4)c3)nc(-c3ccccc3-c3ccccc3)n2)cc1 ; c1ccc(-c2ccccc2-c2nc(-c3cccc(-c4cccc(-c5cccc(-c6nc(-c7ccccc7-c7ccccc7)nc(-c7ccccc7-c7ccccc7)n6)c5)c4)c3)nc(-c3ccccc3-c3ccccc3)n2)cc1 ; c1ccc(-c2ccccc2-c2nc(-c3cccc(-c4cccc(-c5cccc(-c6cccc(-c7nc(-c8ccccc8-c8ccccc8)nc(-c8ccccc8-c8ccccc8)n7)c6)c5)c4)c3)nc(-c3ccccc3-c3ccccc3)n2)cc1]

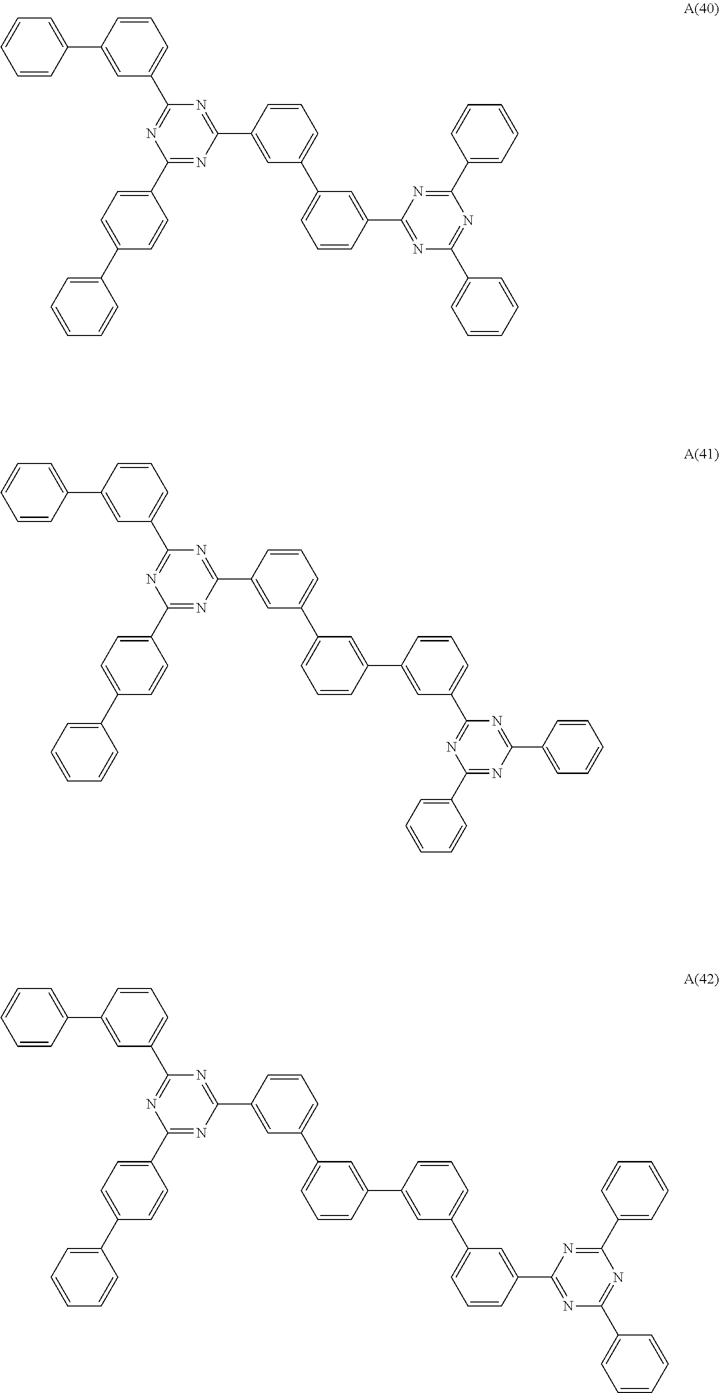 [SMILES: c1ccc(-c2ccc(-c3nc(-c4cccc(-c5ccccc5)c4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)cc2)cc1]

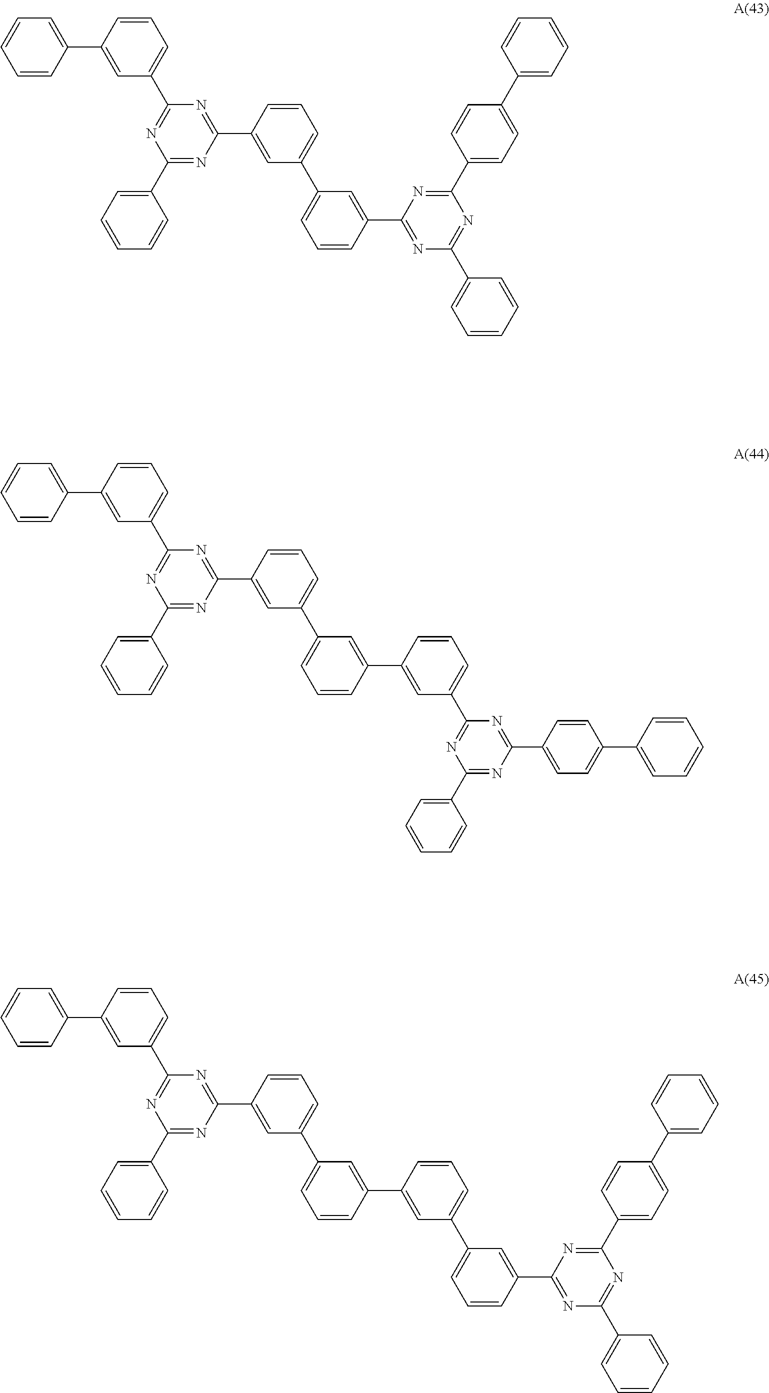 [SMILES: c1ccc(-c2ccc(-c3nc(-c4ccccc4)nc(-c4cccc(-c5cccc(-c6nc(-c7ccccc7)nc(-c7cccc(-c8ccccc8)c7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1]

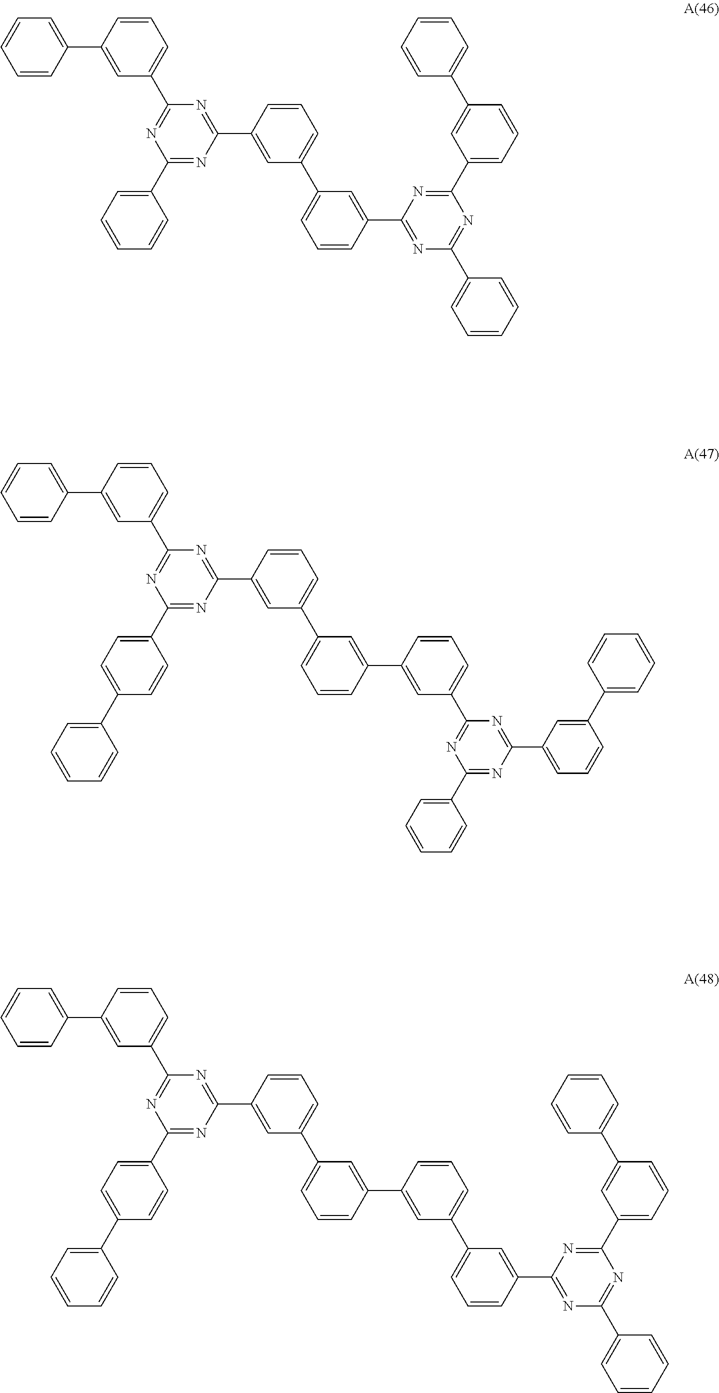 [SMILES: c1ccc(-c2cccc(-c3nc(-c4ccccc4)nc(-c4cccc(-c5cccc(-c6nc(-c7ccccc7)nc(-c7cccc(-c8ccccc8)c7)n6)c5)c4)n3)c2)cc1 ; c1ccc(-c2ccc(-c3nc(-c4cccc(-c5ccccc5)c4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1]

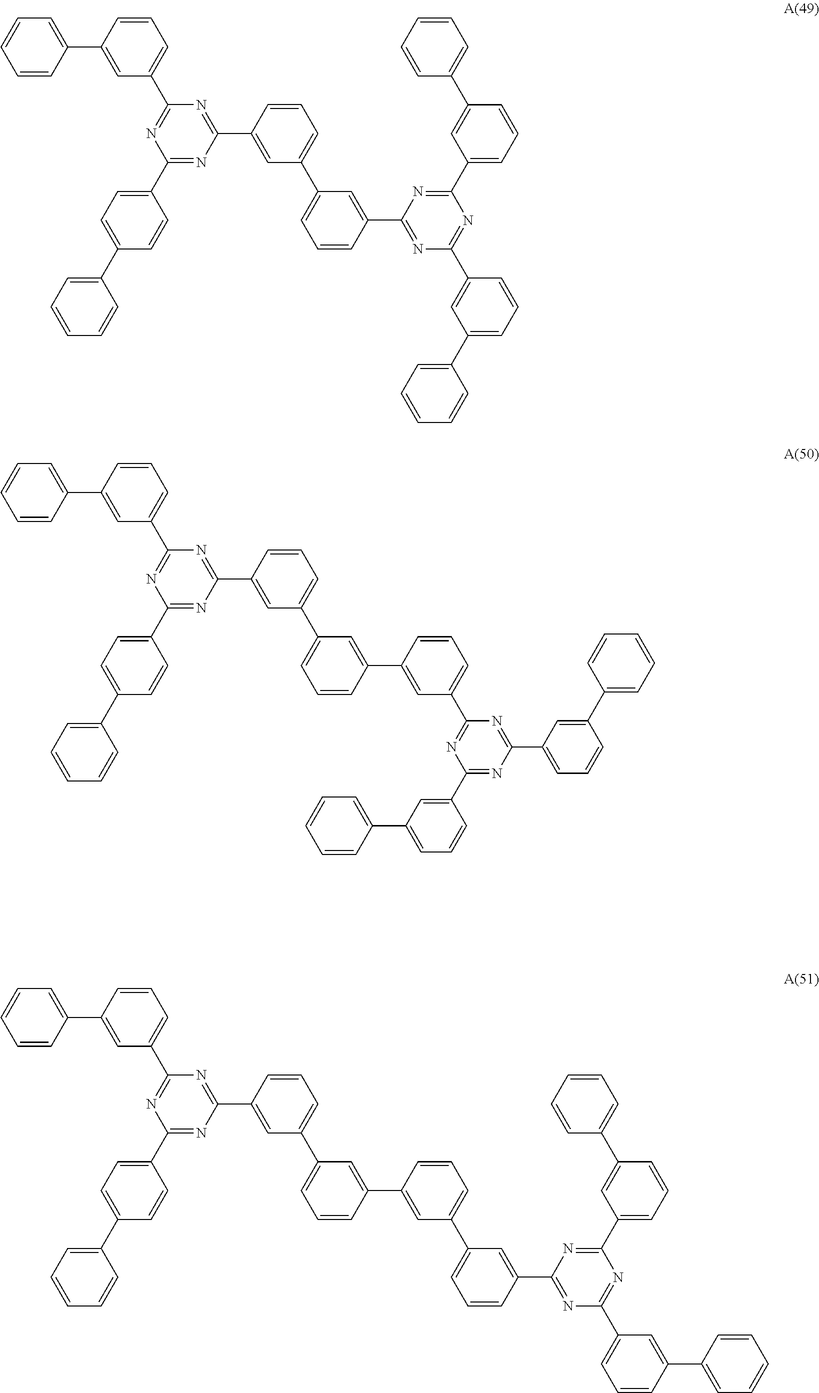 [SMILES: c1ccc(-c2ccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)cc2)cc1]

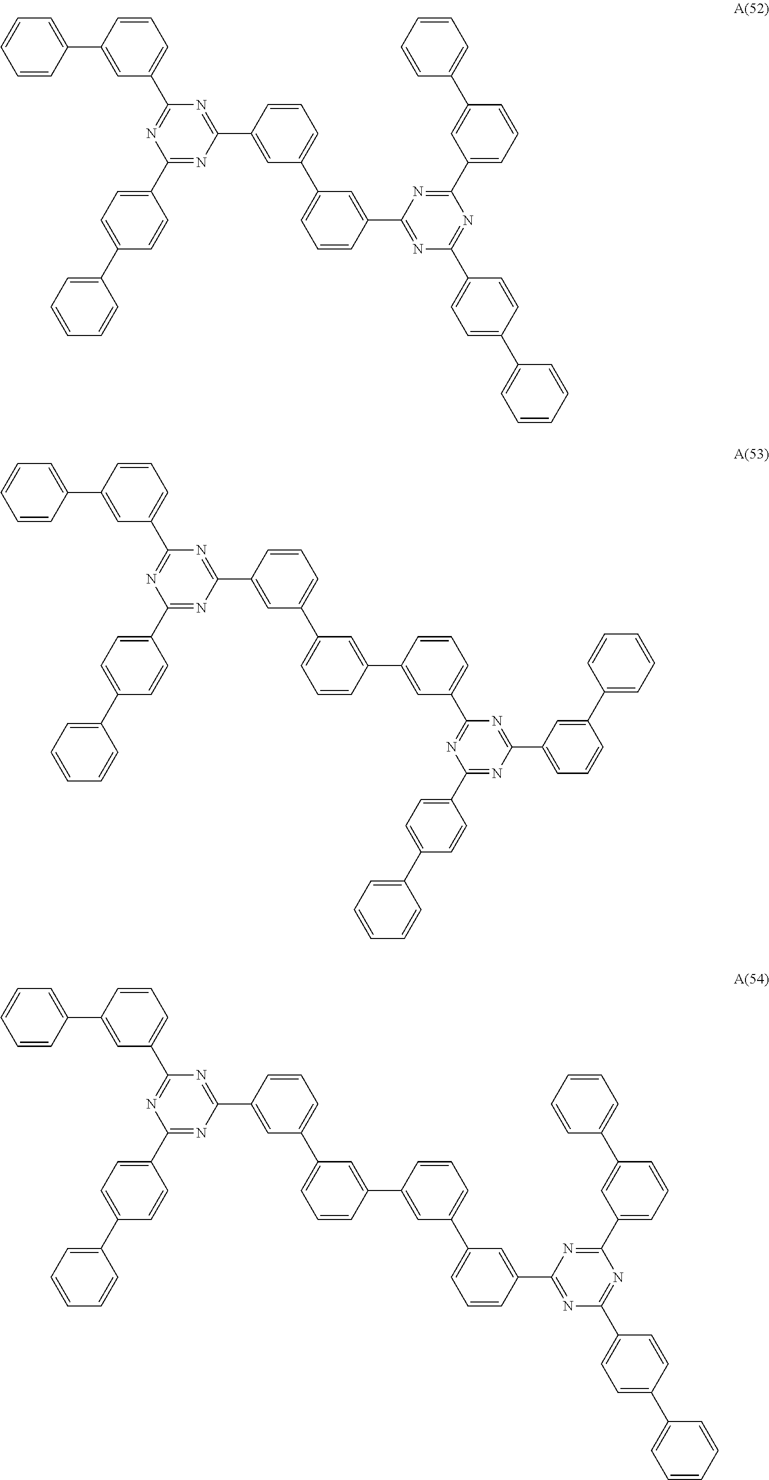 [SMILES: c1ccc(-c2ccc(-c3nc(-c4cccc(-c5ccccc5)c4)nc(-c4cccc(-c5cccc(-c6nc(-c7ccc(-c8ccccc8)cc7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccc(-c9ccccc9)cc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccc(-c%10ccccc%10)cc9)nc(-c9cccc(-c%10ccccc%10)c9)n8)c7)c6)c5)c4)n3)cc2)cc1]

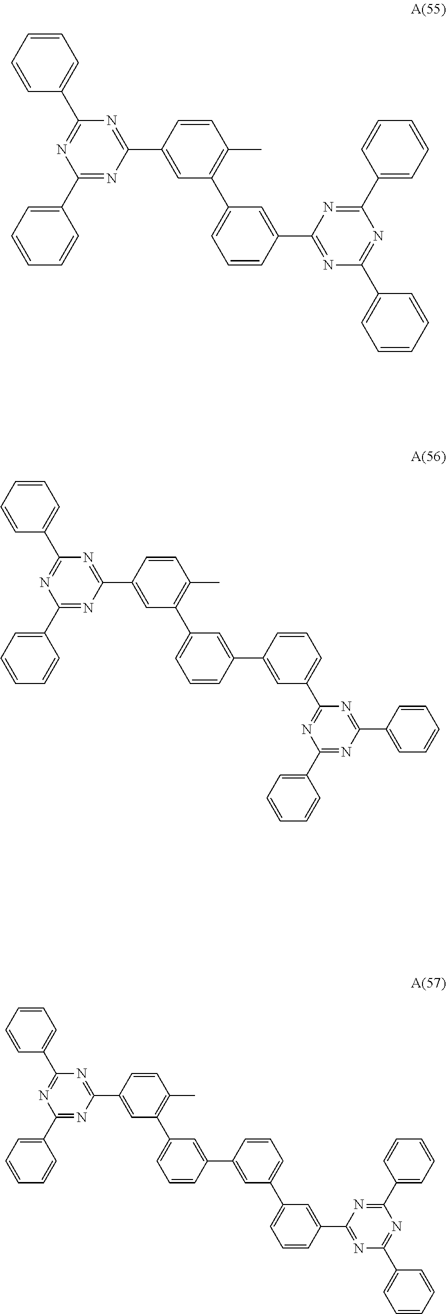 [SMILES: Cc1ccc(-c2nc(-c3ccccc3)nc(-c3ccccc3)n2)cc1-c1cccc(-c2nc(-c3ccccc3)nc(-c3ccccc3)n2)c1 ; Cc1ccc(-c2nc(-c3ccccc3)nc(-c3ccccc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccccc3)n2)cc1-c1cccc(-c2cccc(-c3cccc(-c4nc(-c5ccccc5)nc(-c5ccccc5)n4)c3)c2)c1]

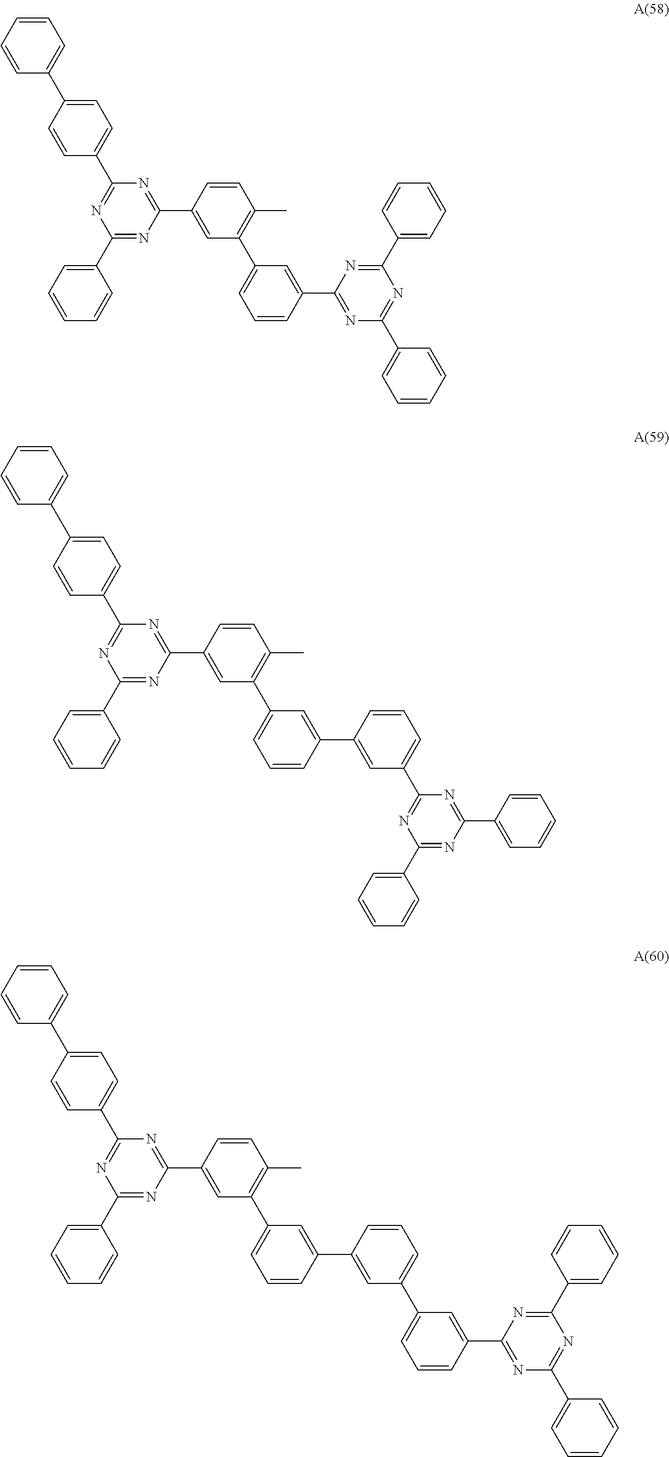 [SMILES: Cc1ccc(-c2nc(-c3ccccc3)nc(-c3ccc(-c4ccccc4)cc3)n2)cc1-c1cccc(-c2nc(-c3ccccc3)nc(-c3ccccc3)n2)c1 ; Cc1ccc(-c2nc(-c3ccccc3)nc(-c3ccc(-c4ccccc4)cc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccc(-c4ccccc4)cc3)n2)cc1-c1cccc(-c2cccc(-c3cccc(-c4nc(-c5ccccc5)nc(-c5ccccc5)n4)c3)c2)c1]

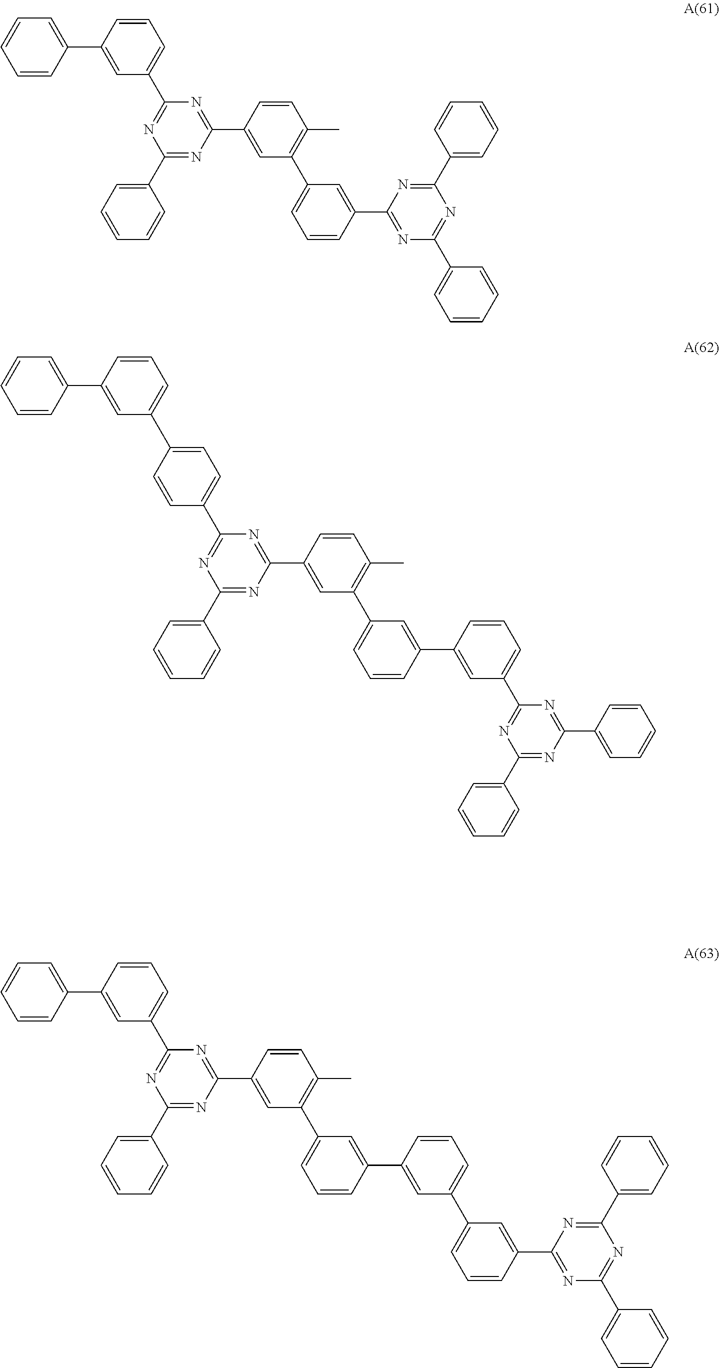 [SMILES: Cc1ccc(-c2nc(-c3ccccc3)nc(-c3cccc(-c4ccccc4)c3)n2)cc1-c1cccc(-c2nc(-c3ccccc3)nc(-c3ccccc3)n2)c1 ; Cc1ccc(-c2nc(-c3ccccc3)nc(-c3ccc(-c4cccc(-c5ccccc5)c4)cc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3cccc(-c4ccccc4)c3)n2)cc1-c1cccc(-c2cccc(-c3cccc(-c4nc(-c5ccccc5)nc(-c5ccccc5)n4)c3)c2)c1]

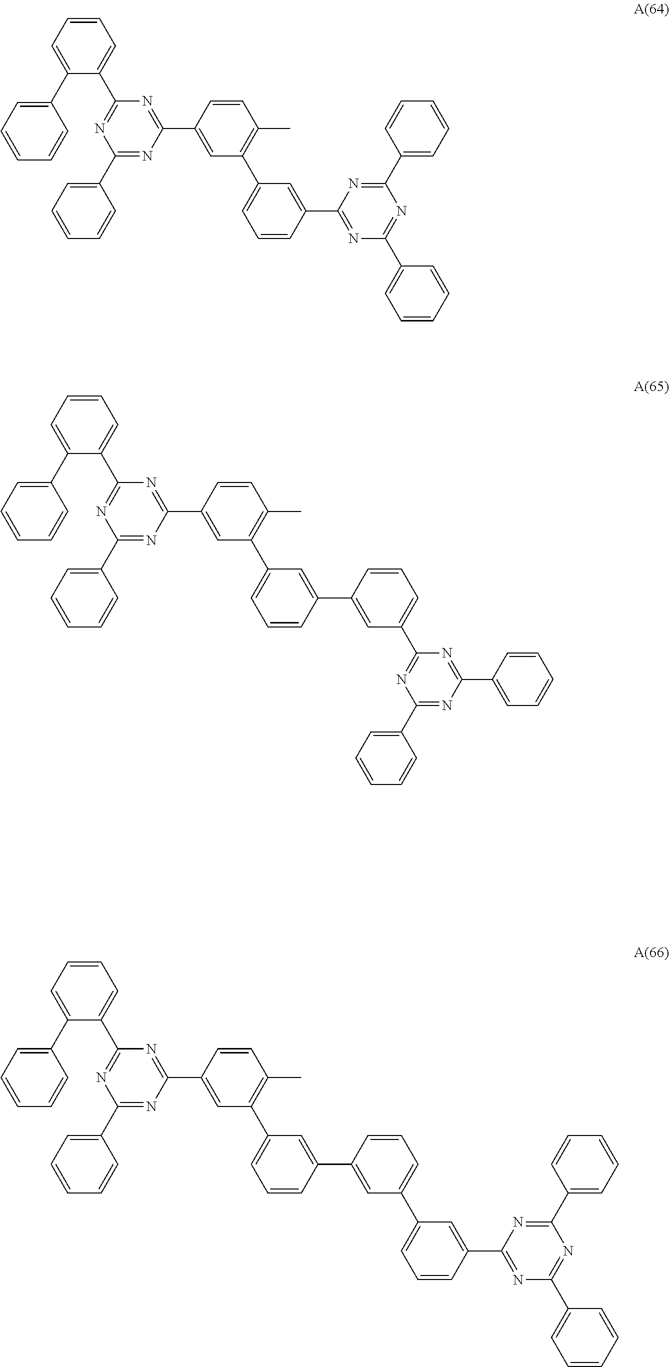 [SMILES: Cc1ccc(-c2nc(-c3ccccc3)nc(-c3ccccc3-c3ccccc3)n2)cc1-c1cccc(-c2nc(-c3ccccc3)nc(-c3ccccc3)n2)c1 ; Cc1ccc(-c2nc(-c3ccccc3)nc(-c3ccccc3-c3ccccc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccccc3-c3ccccc3)n2)cc1-c1cccc(-c2cccc(-c3cccc(-c4nc(-c5ccccc5)nc(-c5ccccc5)n4)c3)c2)c1]

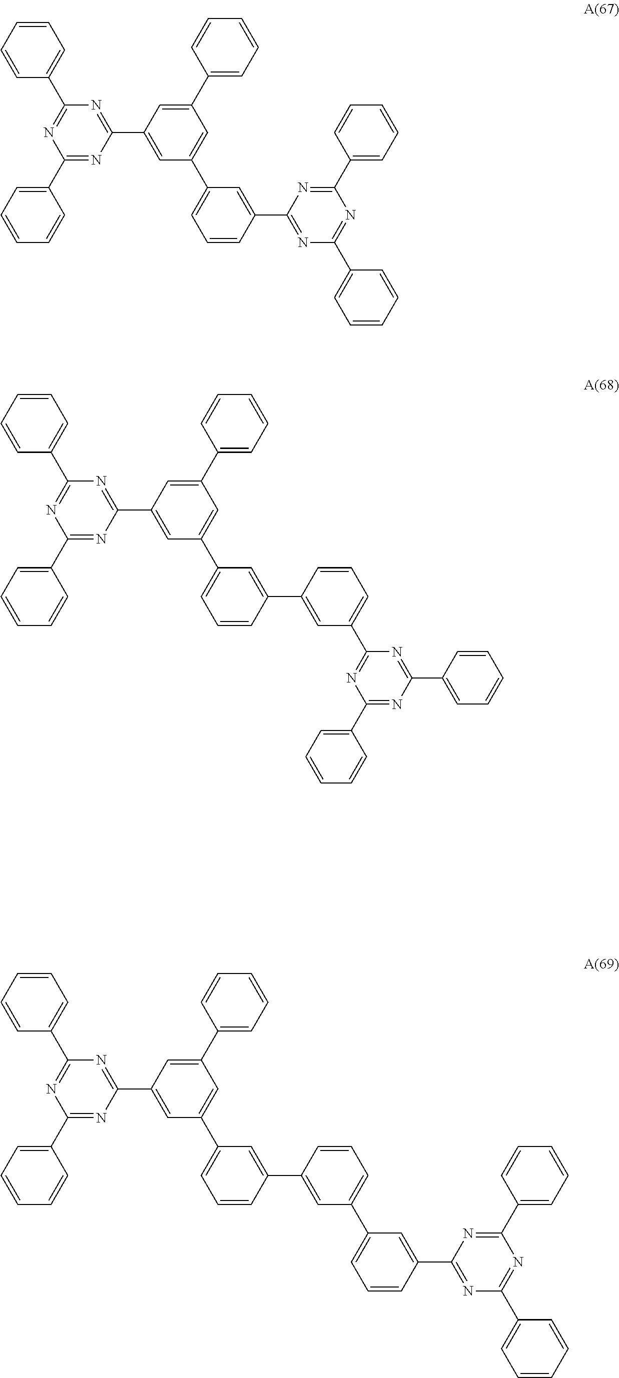 [SMILES: c1ccc(-c2cc(-c3cccc(-c4nc(-c5ccccc5)nc(-c5ccccc5)n4)c3)cc(-c3nc(-c4ccccc4)nc(-c4ccccc4)n3)c2)cc1 ; c1ccc(-c2cc(-c3cccc(-c4cccc(-c5nc(-c6ccccc6)nc(-c6ccccc6)n5)c4)c3)cc(-c3nc(-c4ccccc4)nc(-c4ccccc4)n3)c2)cc1 ; c1ccc(-c2cc(-c3cccc(-c4cccc(-c5cccc(-c6nc(-c7ccccc7)nc(-c7ccccc7)n6)c5)c4)c3)cc(-c3nc(-c4ccccc4)nc(-c4ccccc4)n3)c2)cc1]

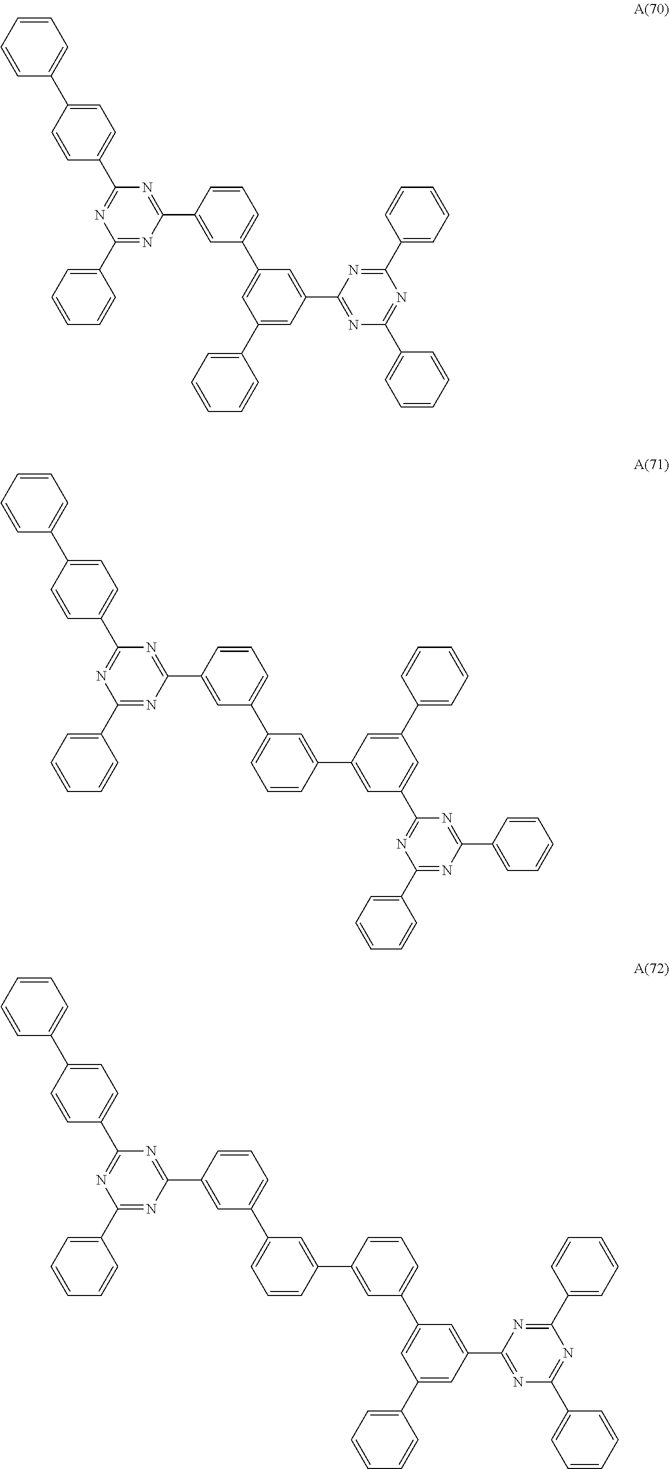 [SMILES: c1ccc(-c2ccc(-c3nc(-c4ccccc4)nc(-c4cccc(-c5cc(-c6ccccc6)cc(-c6nc(-c7ccccc7)nc(-c7ccccc7)n6)c5)c4)n3)cc2)cc1 ; c1ccc(-c2ccc(-c3nc(-c4ccccc4)nc(-c4cccc(-c5cccc(-c6cc(-c7ccccc7)cc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cc(-c8ccccc8)cc(-c8nc(-c9ccccc9)nc(-c9ccccc9)n8)c7)c6)c5)c4)n3)cc2)cc1]

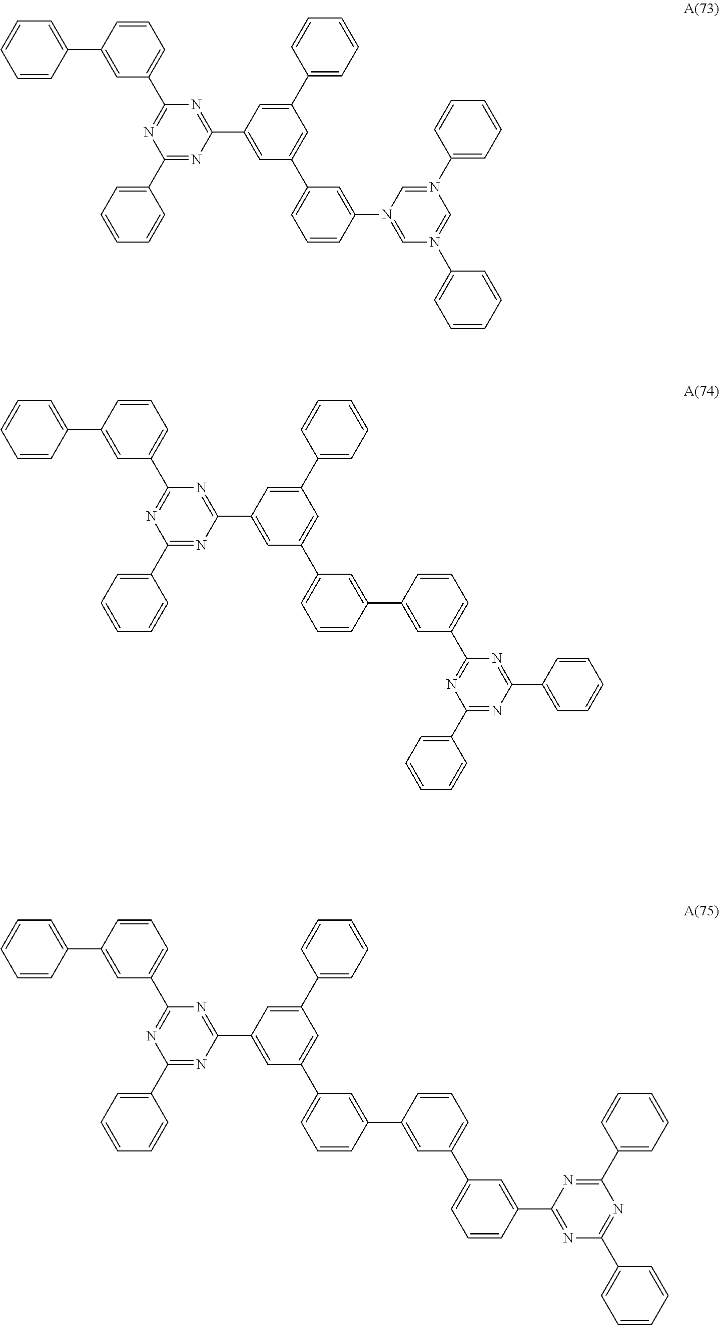

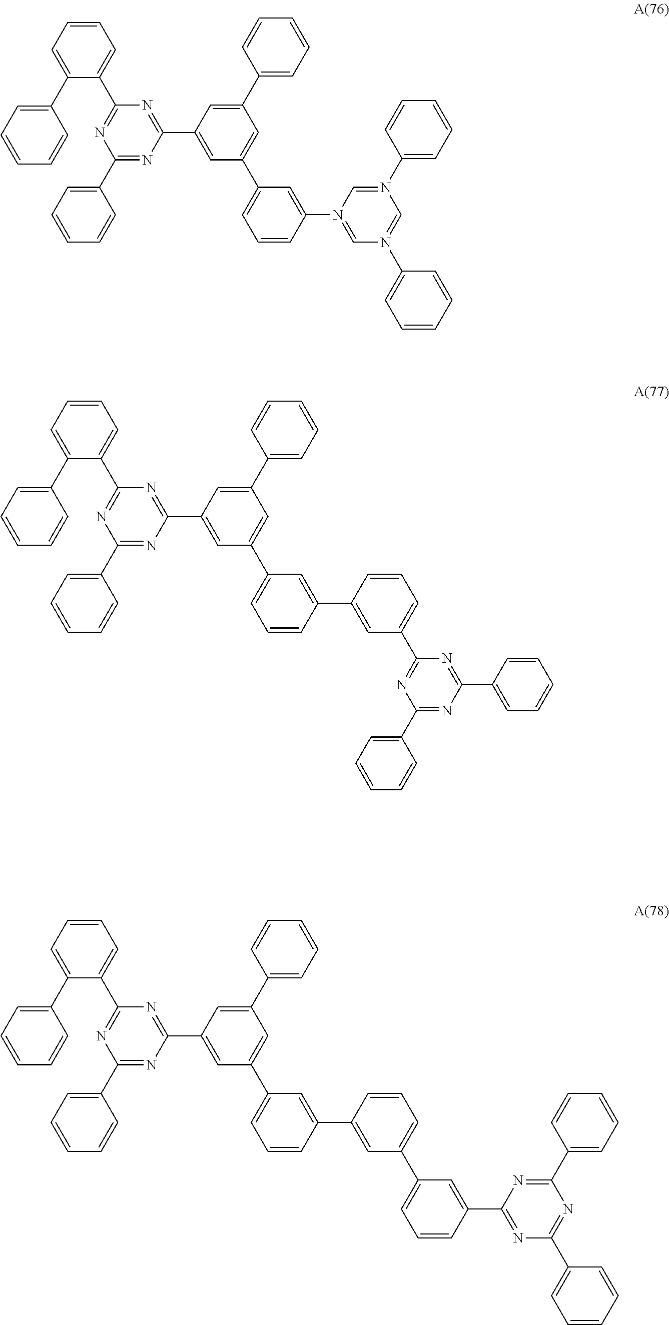

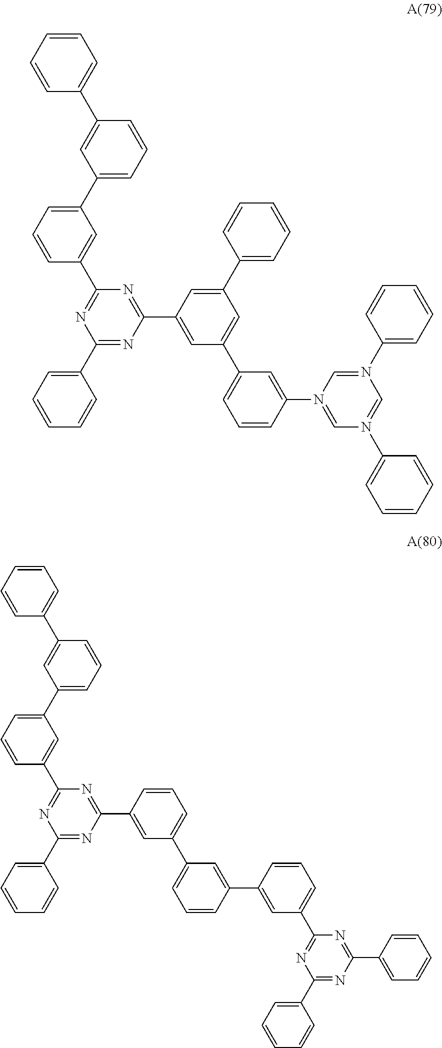

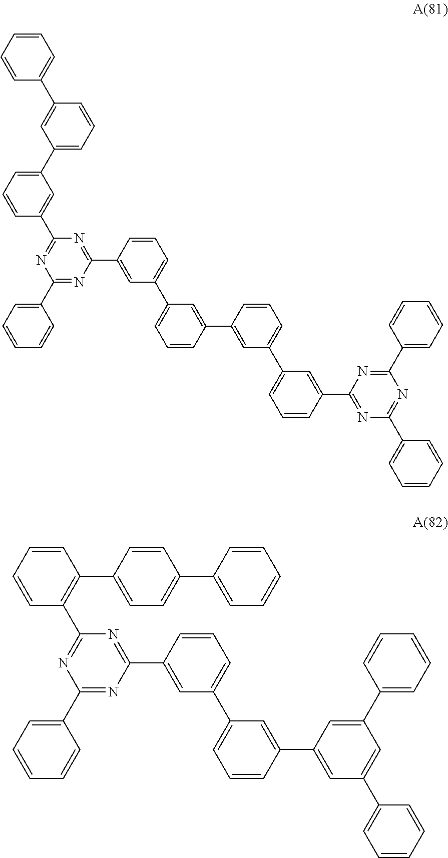 [SMILES: c1ccc(-c2cccc(-c3cccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)c3)c2)cc1 ; c1ccc(-c2ccc(-c3ccccc3-c3nc(-c4ccccc4)nc(-c4cccc(-c5cccc(-c6cc(-c7ccccc7)cc(-c7ccccc7)c6)c5)c4)n3)cc2)cc1]

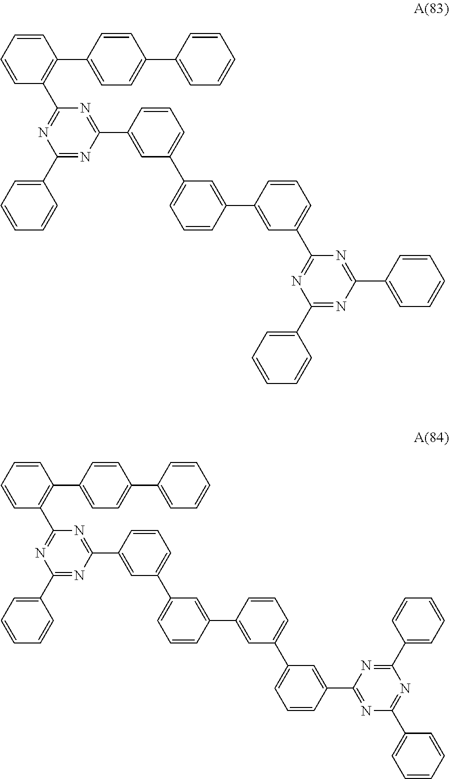 [SMILES: c1ccc(-c2ccc(-c3ccccc3-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3ccccc3-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

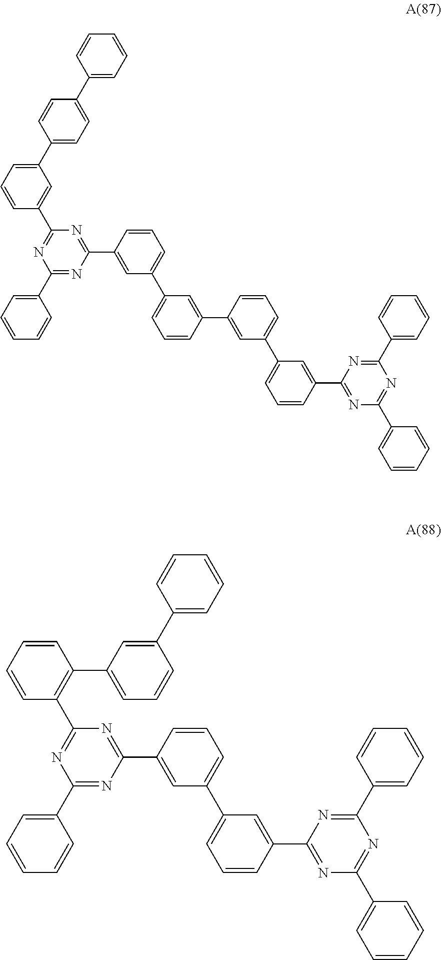 [SMILES: c1ccc(-c2ccc(-c3cccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)c3)cc2)cc1 ; c1ccc(-c2cccc(-c3ccccc3-c3nc(-c4ccccc4)nc(-c4cccc(-c5cccc(-c6nc(-c7ccccc7)nc(-c7ccccc7)n6)c5)c4)n3)c2)cc1]

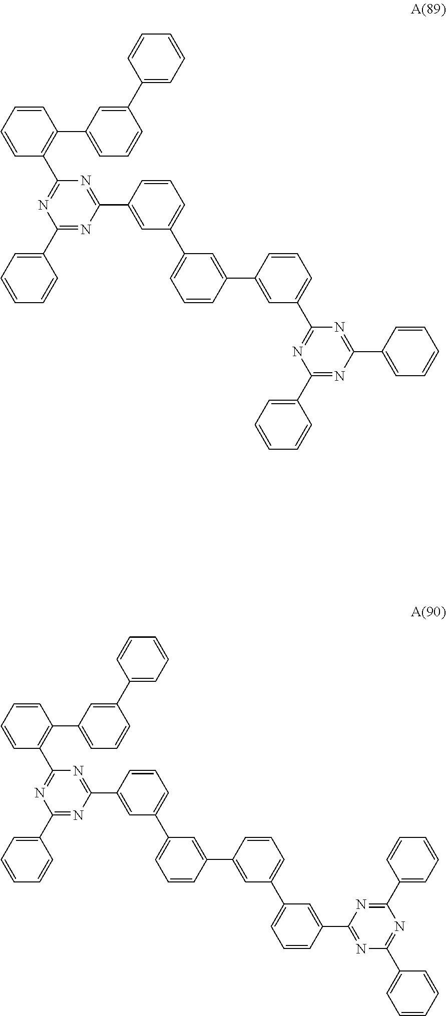 [SMILES: c1ccc(-c2cccc(-c3ccccc3-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3ccccc3-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

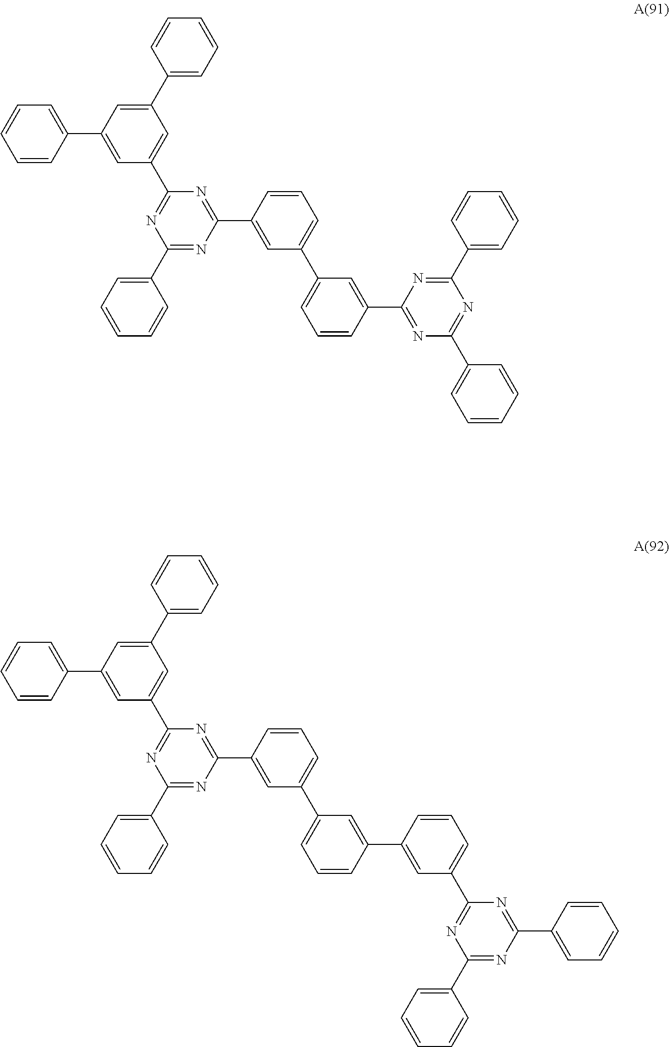 [SMILES: c1ccc(-c2cc(-c3ccccc3)cc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cc(-c3ccccc3)cc(-c3nc(-c4ccccc4)nc(-c4cccc(-c5cccc(-c6cccc(-c7nc(-c8ccccc8)nc(-c8ccccc8)n7)c6)c5)c4)n3)c2)cc1]

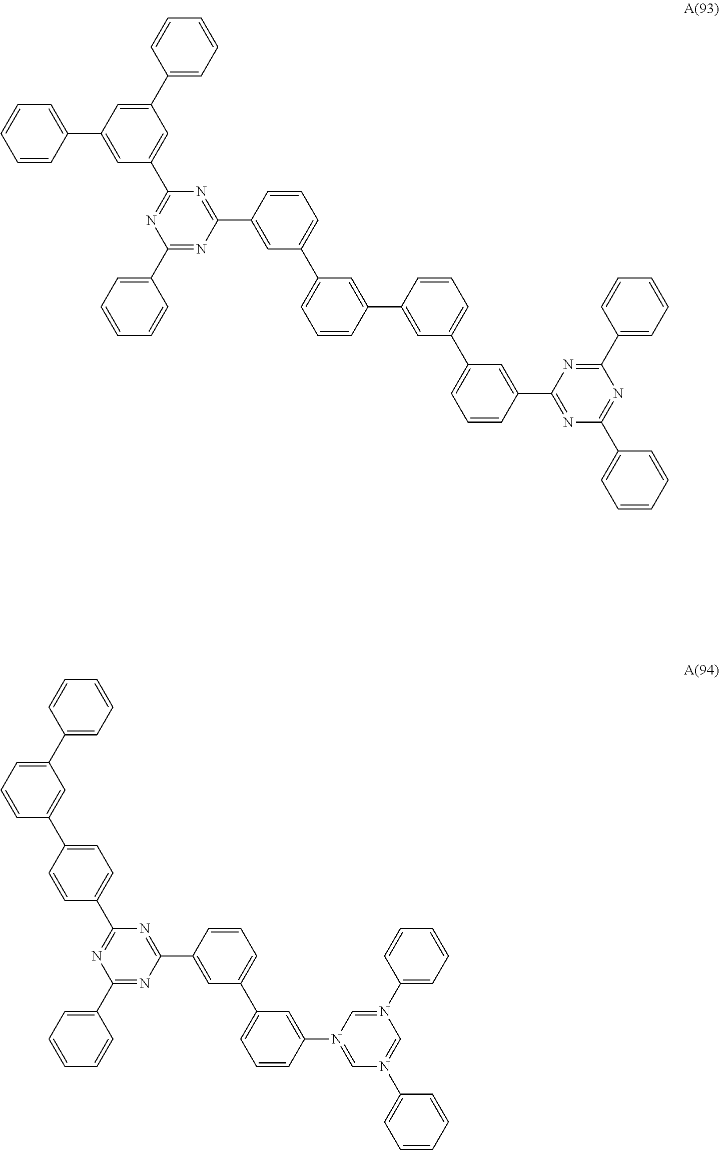

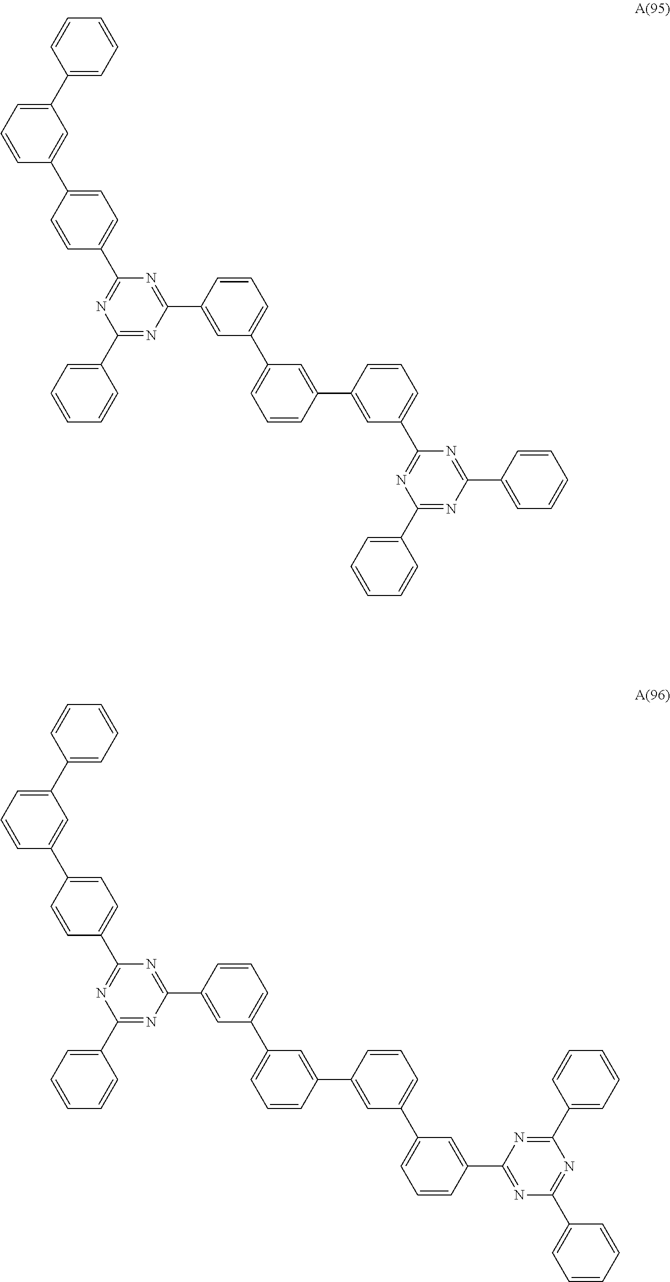 [SMILES: c1ccc(-c2cccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)c2)cc1 ; c1ccc(-c2cccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)c2)cc1]

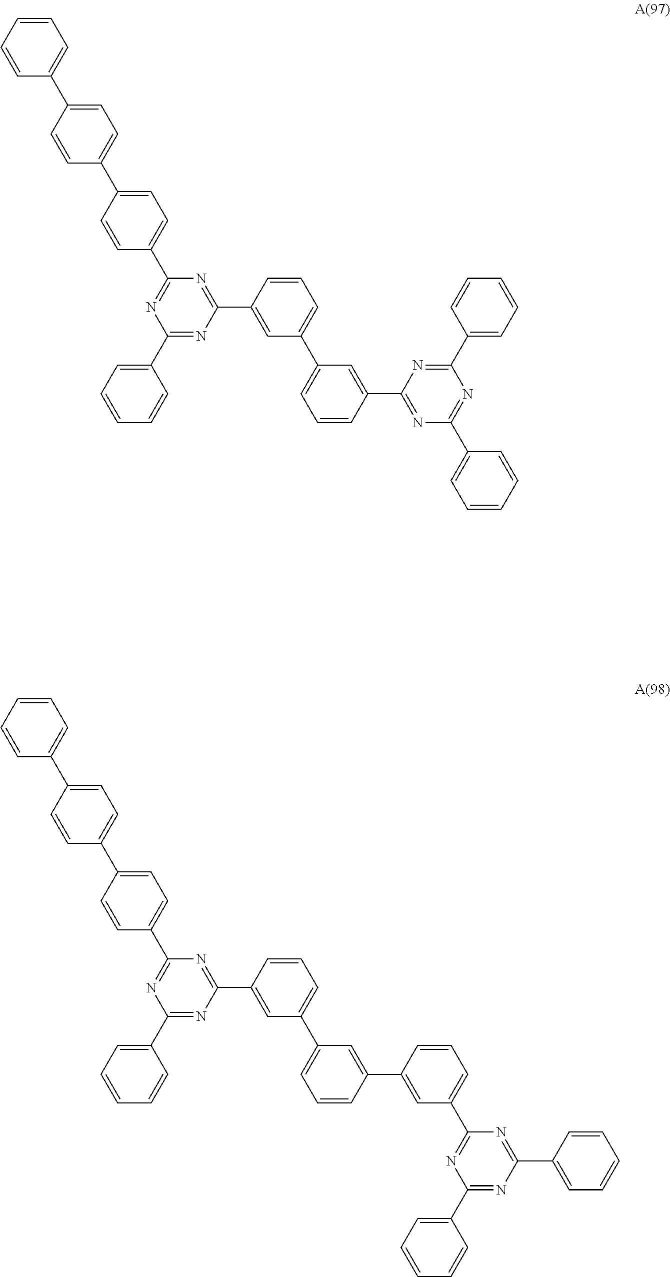 [SMILES: c1ccc(-c2ccc(-c3ccc(-c4nc(-c5ccccc5)nc(-c5cccc(-c6cccc(-c7nc(-c8ccccc8)nc(-c8ccccc8)n7)c6)c5)n4)cc3)cc2)cc1 ; c1ccc(-c2ccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)cc2)cc1]

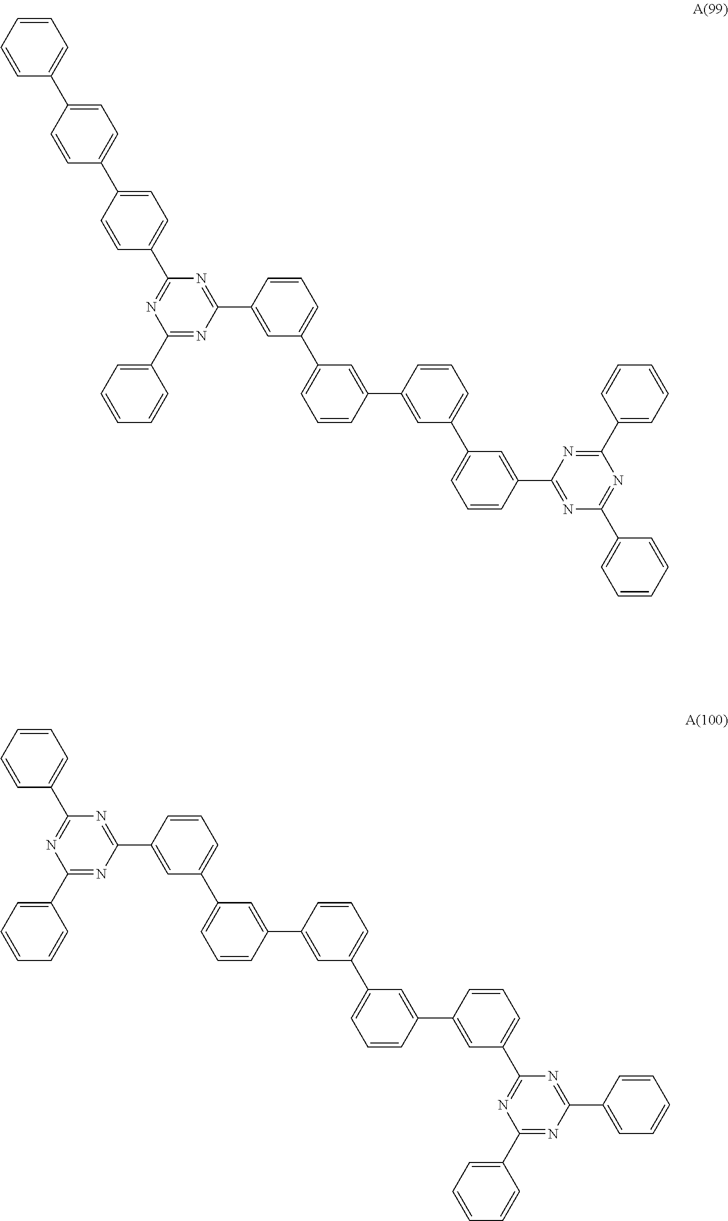 [SMILES: c1ccc(-c2ccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)cc2)cc1 ; c1ccc(-c2nc(-c3ccccc3)nc(-c3cccc(-c4cccc(-c5cccc(-c6cccc(-c7cccc(-c8nc(-c9ccccc9)nc(-c9ccccc9)n8)c7)c6)c5)c4)c3)n2)cc1]

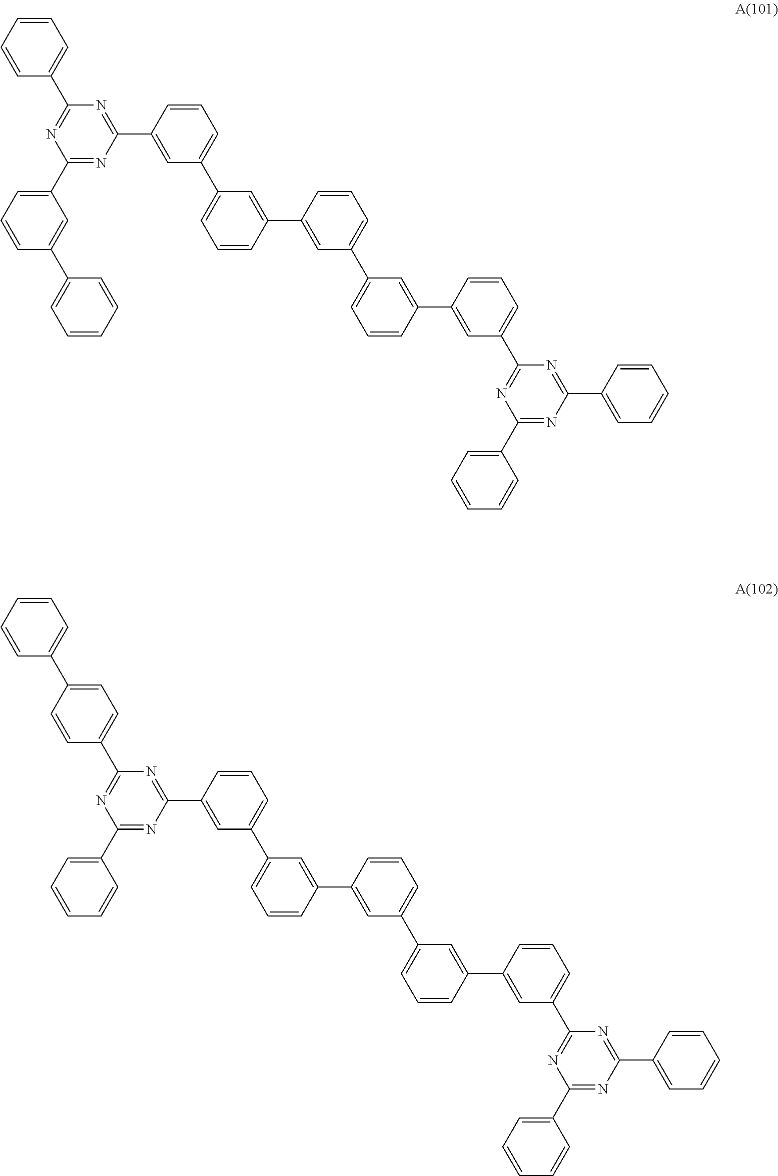 [SMILES: c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1 ; c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1]

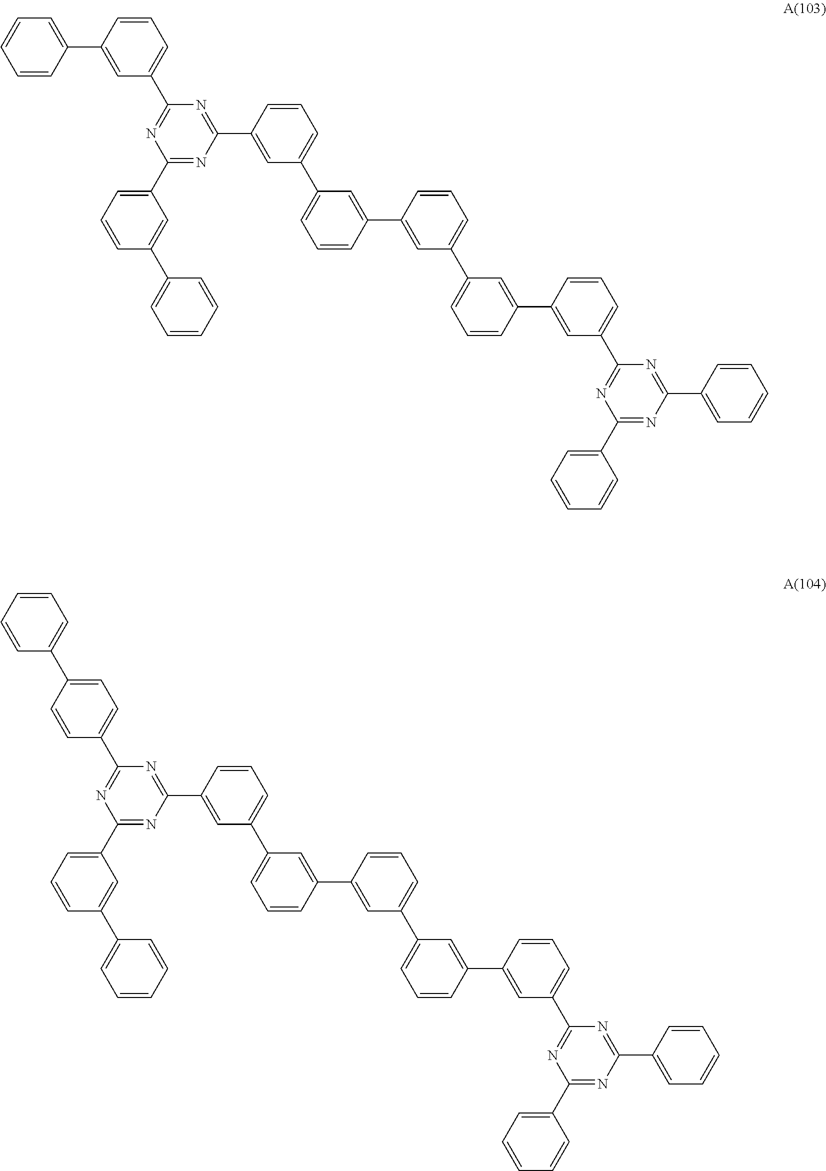 [SMILES: c1ccc(-c2cccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1 ; c1ccc(-c2ccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1]

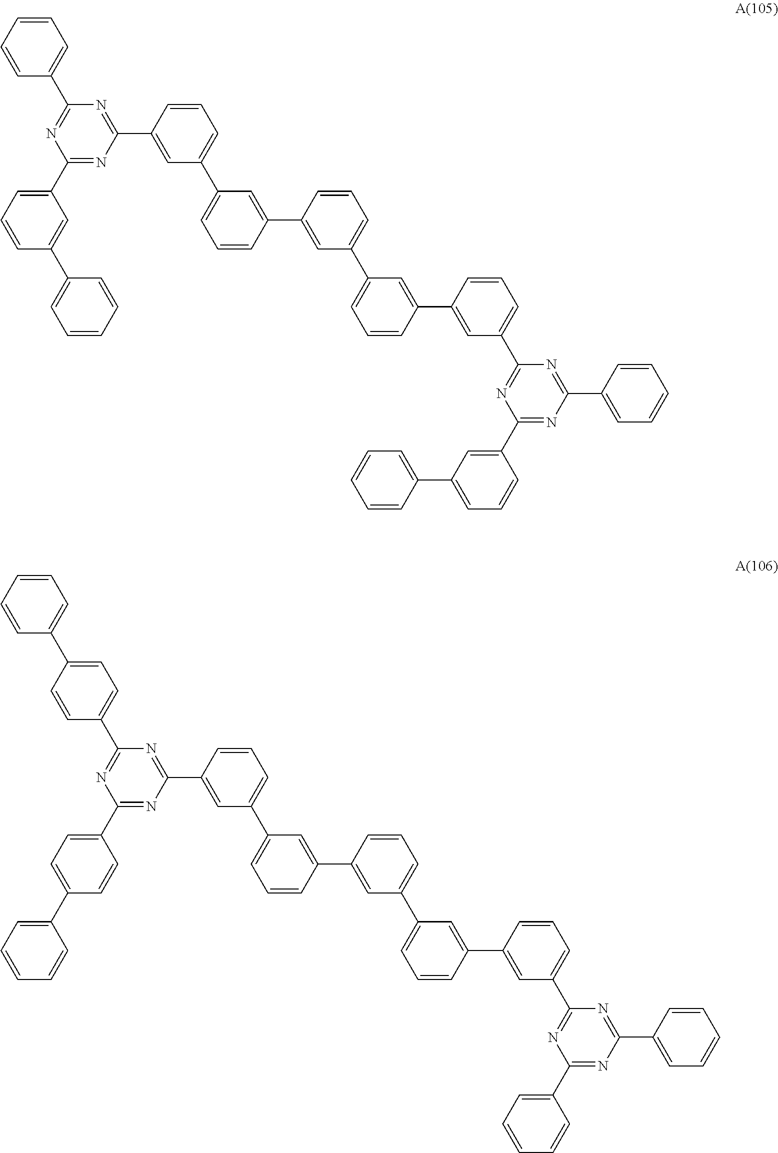 [SMILES: c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)c2)cc1 ; c1ccc(-c2ccc(-c3nc(-c4ccc(-c5ccccc5)cc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1]

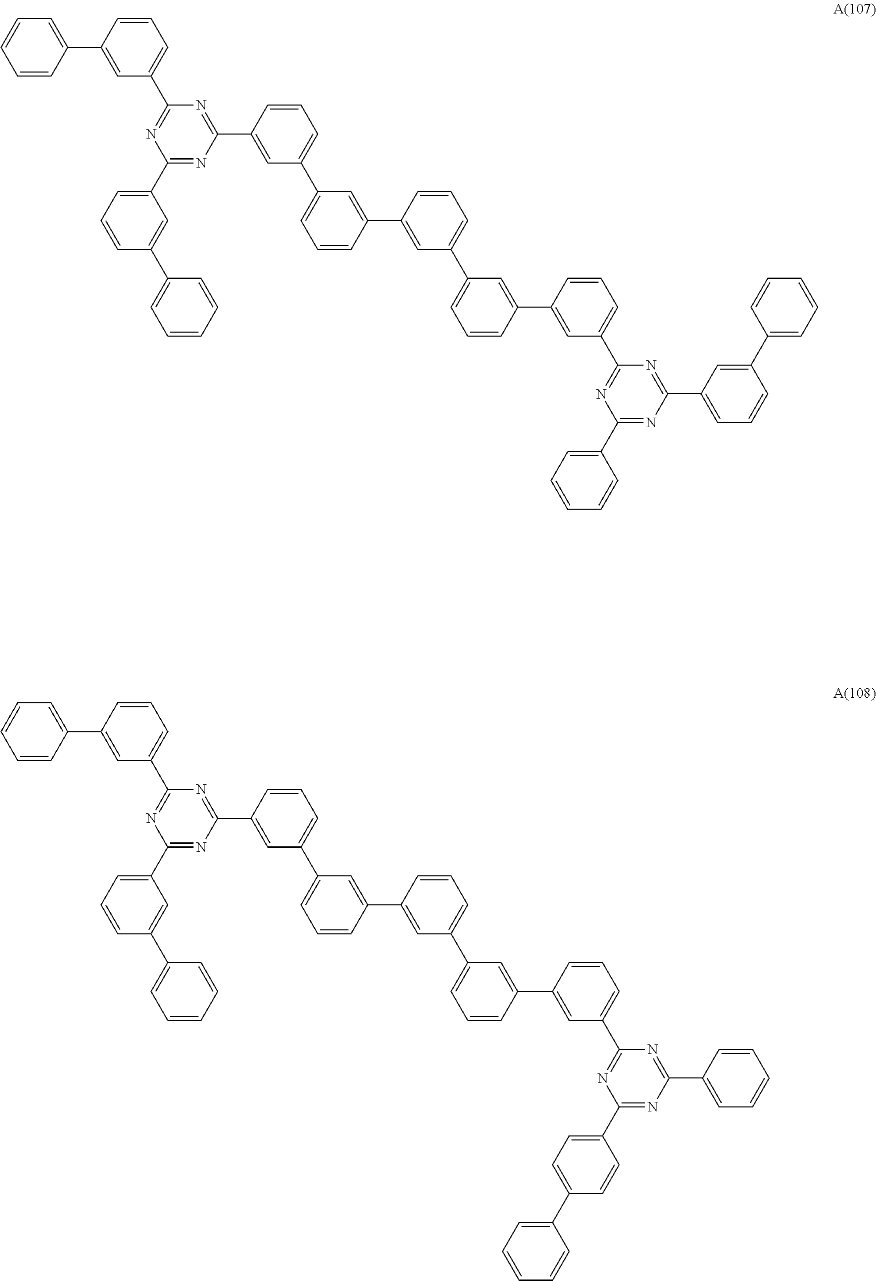 [SMILES: c1ccc(-c2cccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1 ; c1ccc(-c2ccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)cc2)cc1]

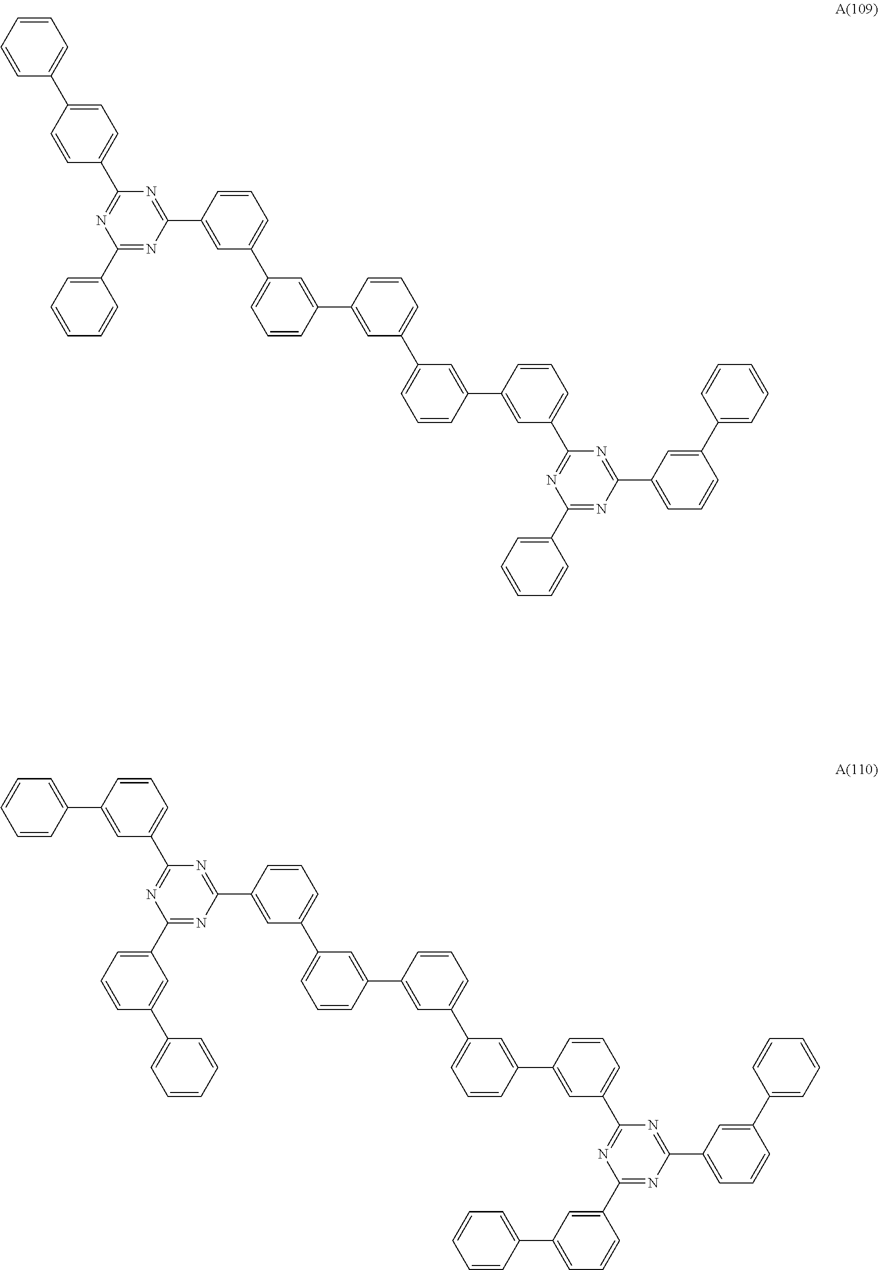 [SMILES: c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)cc2)cc1 ; c1ccc(-c2cccc(-c3nc(-c4cccc(-c5ccccc5)c4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1]

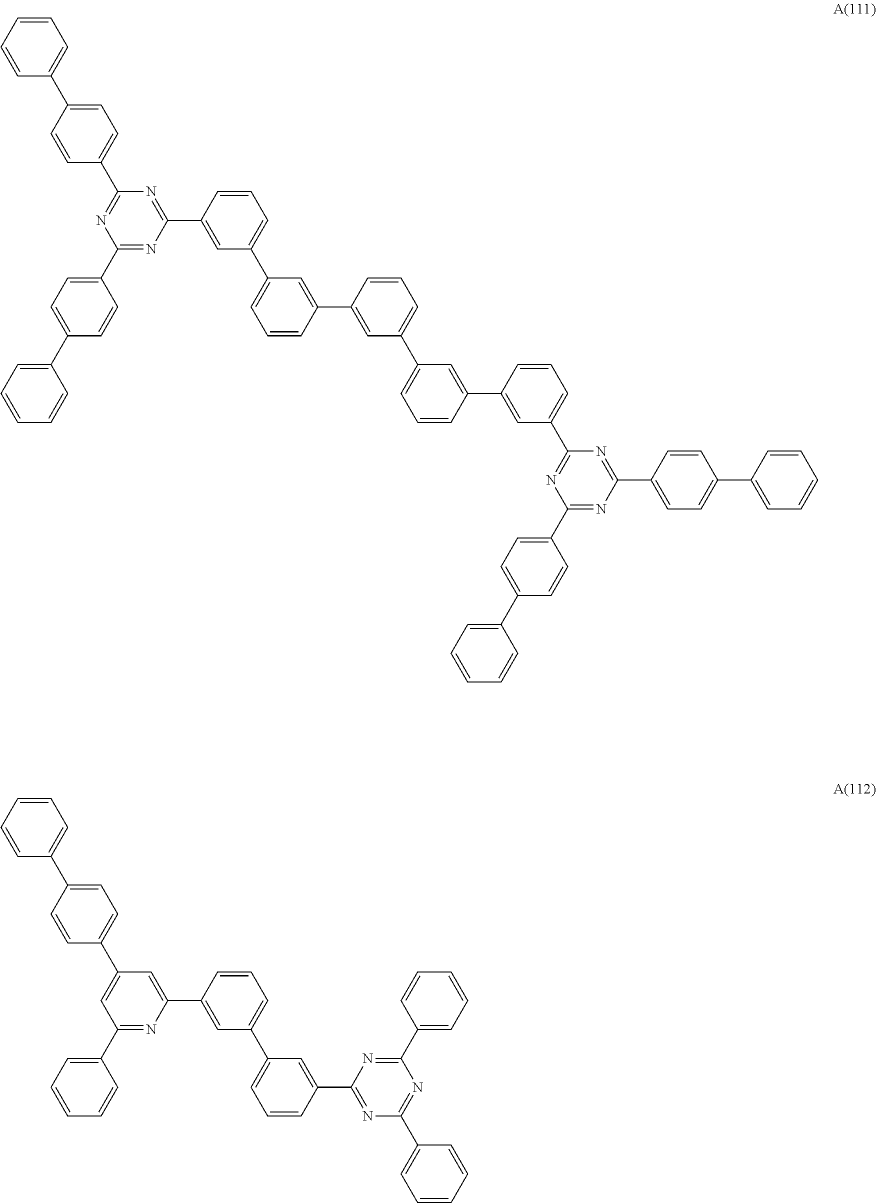 [SMILES: c1ccc(-c2ccc(-c3nc(-c4ccc(-c5ccccc5)cc4)nc(-c4cccc(-c5cccc(-c6cccc(-c7cccc(-c8cccc(-c9nc(-c%10ccc(-c%11ccccc%11)cc%10)nc(-c%10ccc(-c%11ccccc%11)cc%10)n9)c8)c7)c6)c5)c4)n3)cc2)cc1 ; c1ccc(-c2ccc(-c3cc(-c4ccccc4)nc(-c4cccc(-c5cccc(-c6nc(-c7ccccc7)nc(-c7ccccc7)n6)c5)c4)c3)cc2)cc1]

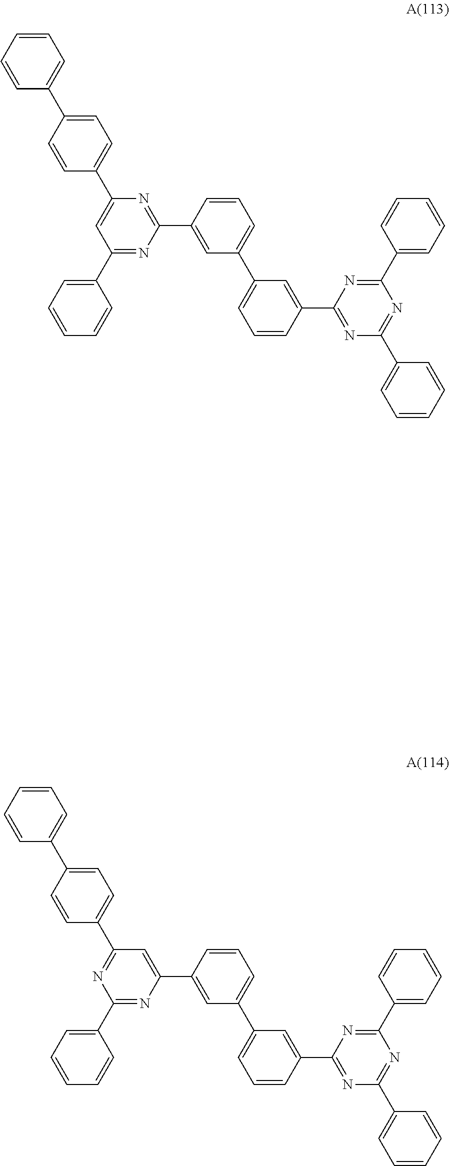 [SMILES: c1ccc(-c2ccc(-c3cc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3cc(-c4cccc(-c5cccc(-c6nc(-c7ccccc7)nc(-c7ccccc7)n6)c5)c4)nc(-c4ccccc4)n3)cc2)cc1]

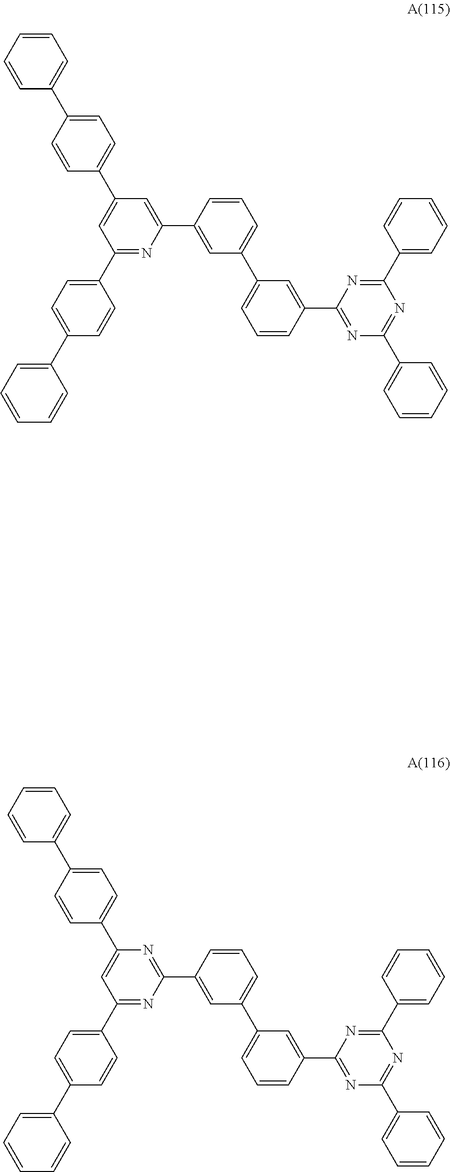 [SMILES: c1ccc(-c2ccc(-c3cc(-c4ccc(-c5ccccc5)cc4)nc(-c4cccc(-c5cccc(-c6nc(-c7ccccc7)nc(-c7ccccc7)n6)c5)c4)c3)cc2)cc1 ; c1ccc(-c2ccc(-c3cc(-c4ccc(-c5ccccc5)cc4)nc(-c4cccc(-c5cccc(-c6nc(-c7ccccc7)nc(-c7ccccc7)n6)c5)c4)n3)cc2)cc1]

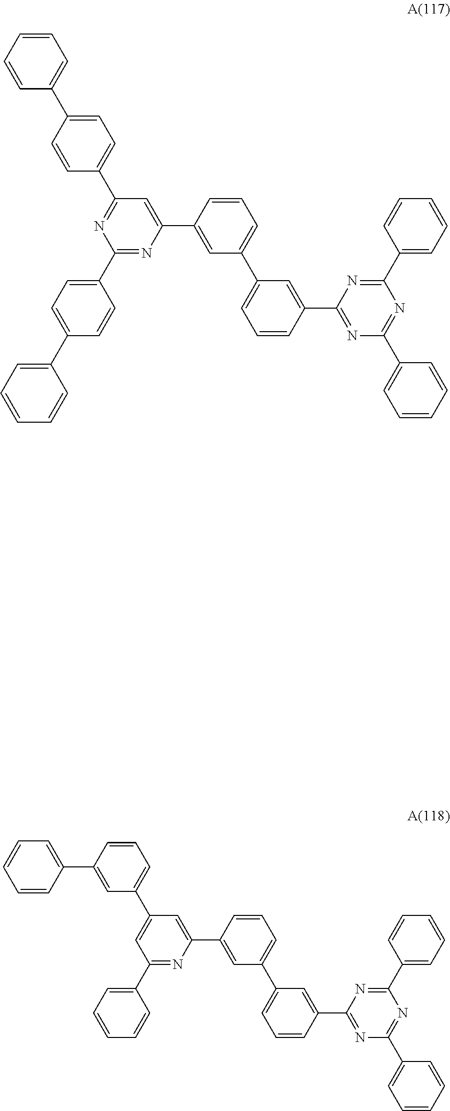 [SMILES: c1ccc(-c2ccc(-c3cc(-c4cccc(-c5cccc(-c6nc(-c7ccccc7)nc(-c7ccccc7)n6)c5)c4)nc(-c4ccc(-c5ccccc5)cc4)n3)cc2)cc1 ; c1ccc(-c2cccc(-c3cc(-c4ccccc4)nc(-c4cccc(-c5cccc(-c6nc(-c7ccccc7)nc(-c7ccccc7)n6)c5)c4)c3)c2)cc1]

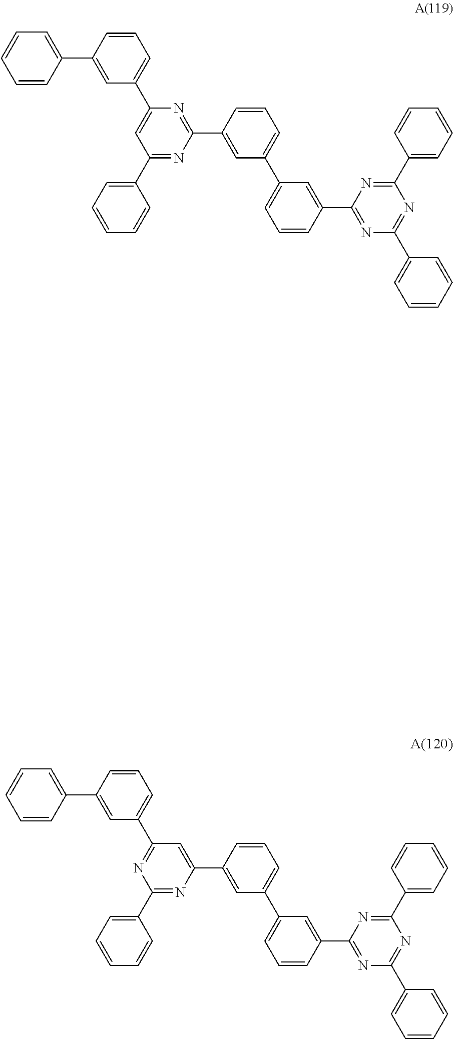 [SMILES: c1ccc(-c2cccc(-c3cc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3cc(-c4cccc(-c5cccc(-c6nc(-c7ccccc7)nc(-c7ccccc7)n6)c5)c4)nc(-c4ccccc4)n3)c2)cc1]

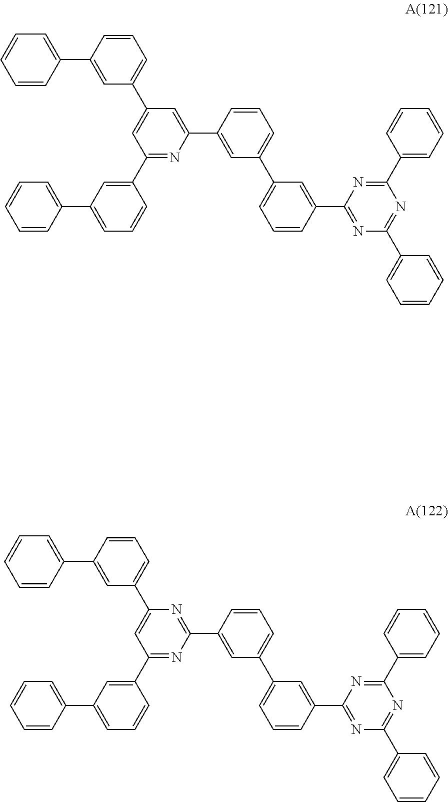 [SMILES: c1ccc(-c2cccc(-c3cc(-c4cccc(-c5ccccc5)c4)nc(-c4cccc(-c5cccc(-c6nc(-c7ccccc7)nc(-c7ccccc7)n6)c5)c4)c3)c2)cc1 ; c1ccc(-c2cccc(-c3cc(-c4cccc(-c5ccccc5)c4)nc(-c4cccc(-c5cccc(-c6nc(-c7ccccc7)nc(-c7ccccc7)n6)c5)c4)n3)c2)cc1]

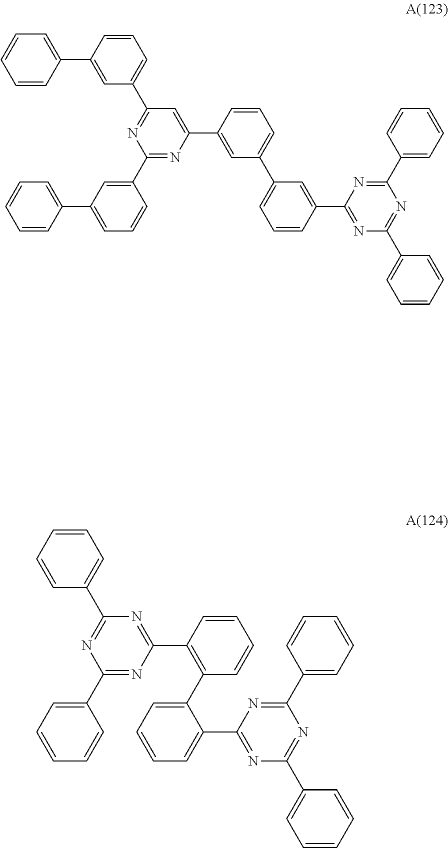 [SMILES: c1ccc(-c2cccc(-c3cc(-c4cccc(-c5cccc(-c6nc(-c7ccccc7)nc(-c7ccccc7)n6)c5)c4)nc(-c4cccc(-c5ccccc5)c4)n3)c2)cc1 ; c1ccc(-c2nc(-c3ccccc3)nc(-c3ccccc3-c3ccccc3-c3nc(-c4ccccc4)nc(-c4ccccc4)n3)n2)cc1]

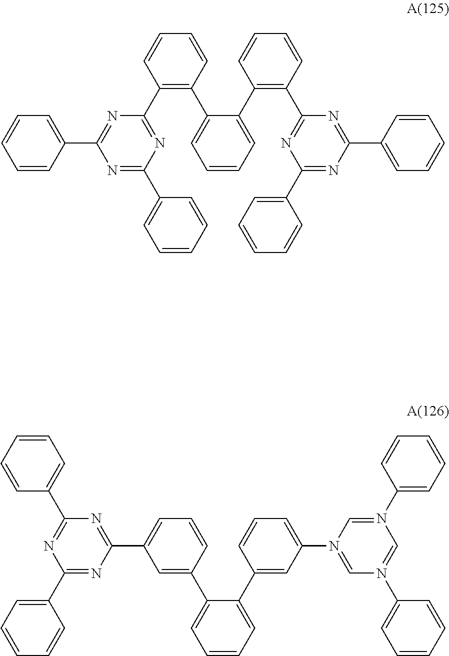

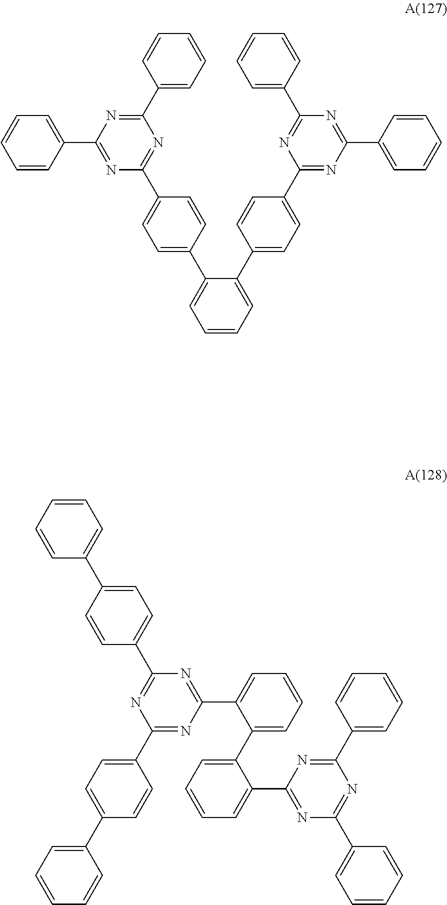 [SMILES: c1ccc(-c2nc(-c3ccccc3)nc(-c3ccc(-c4ccccc4-c4ccc(-c5nc(-c6ccccc6)nc(-c6ccccc6)n5)cc4)cc3)n2)cc1 ; c1ccc(-c2ccc(-c3nc(-c4ccc(-c5ccccc5)cc4)nc(-c4ccccc4-c4ccccc4-c4nc(-c5ccccc5)nc(-c5ccccc5)n4)n3)cc2)cc1]

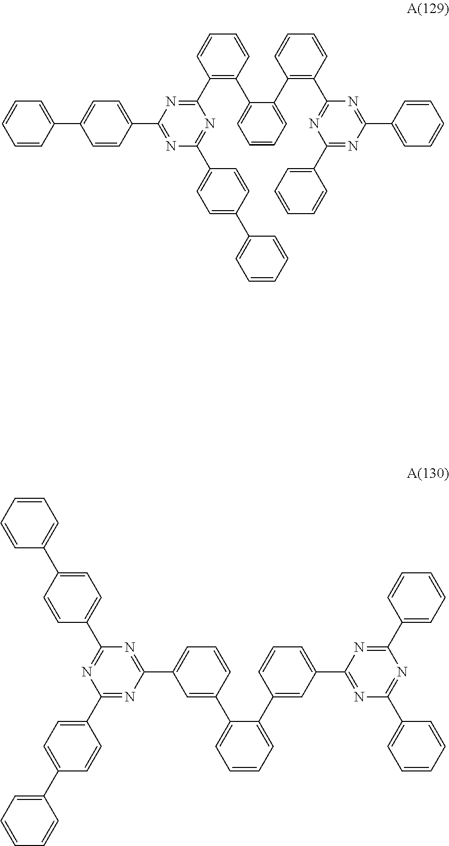 [SMILES: c1ccc(-c2ccc(-c3nc(-c4ccc(-c5ccccc5)cc4)nc(-c4ccccc4-c4ccccc4-c4ccccc4-c4nc(-c5ccccc5)nc(-c5ccccc5)n4)n3)cc2)cc1 ; c1ccc(-c2ccc(-c3nc(-c4ccc(-c5ccccc5)cc4)nc(-c4cccc(-c5ccccc5-c5cccc(-c6nc(-c7ccccc7)nc(-c7ccccc7)n6)c5)c4)n3)cc2)cc1]

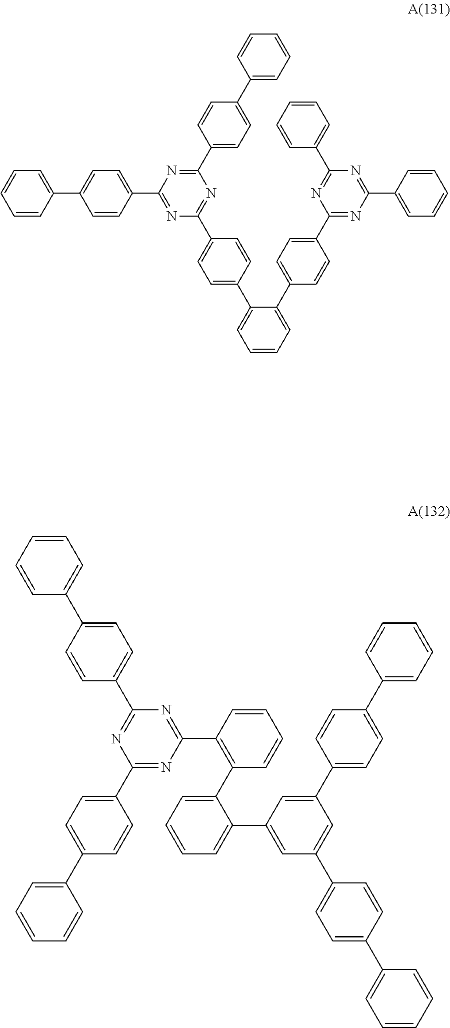 [SMILES: c1ccc(-c2ccc(-c3nc(-c4ccc(-c5ccccc5)cc4)nc(-c4ccc(-c5ccccc5-c5ccc(-c6nc(-c7ccccc7)nc(-c7ccccc7)n6)cc5)cc4)n3)cc2)cc1 ; c1ccc(-c2ccc(-c3cc(-c4ccc(-c5ccccc5)cc4)cc(-c4ccccc4-c4ccccc4-c4nc(-c5ccc(-c6ccccc6)cc5)nc(-c5ccc(-c6ccccc6)cc5)n4)c3)cc2)cc1]

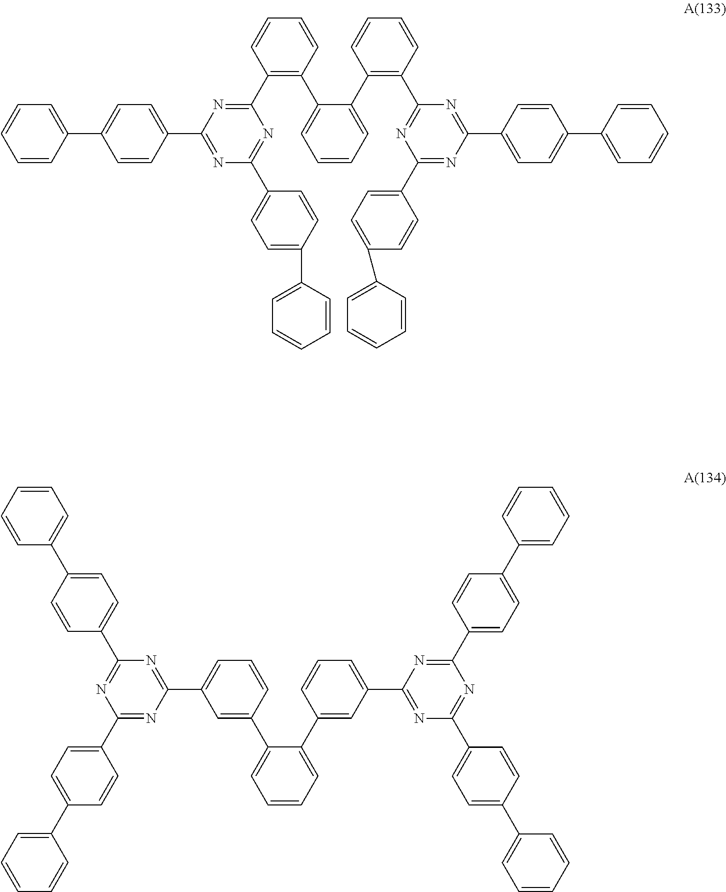 [SMILES: c1ccc(-c2ccc(-c3nc(-c4ccc(-c5ccccc5)cc4)nc(-c4ccccc4-c4ccccc4-c4ccccc4-c4nc(-c5ccc(-c6ccccc6)cc5)nc(-c5ccc(-c6ccccc6)cc5)n4)n3)cc2)cc1 ; c1ccc(-c2ccc(-c3nc(-c4ccc(-c5ccccc5)cc4)nc(-c4cccc(-c5ccccc5-c5cccc(-c6nc(-c7ccc(-c8ccccc8)cc7)nc(-c7ccc(-c8ccccc8)cc7)n6)c5)c4)n3)cc2)cc1]

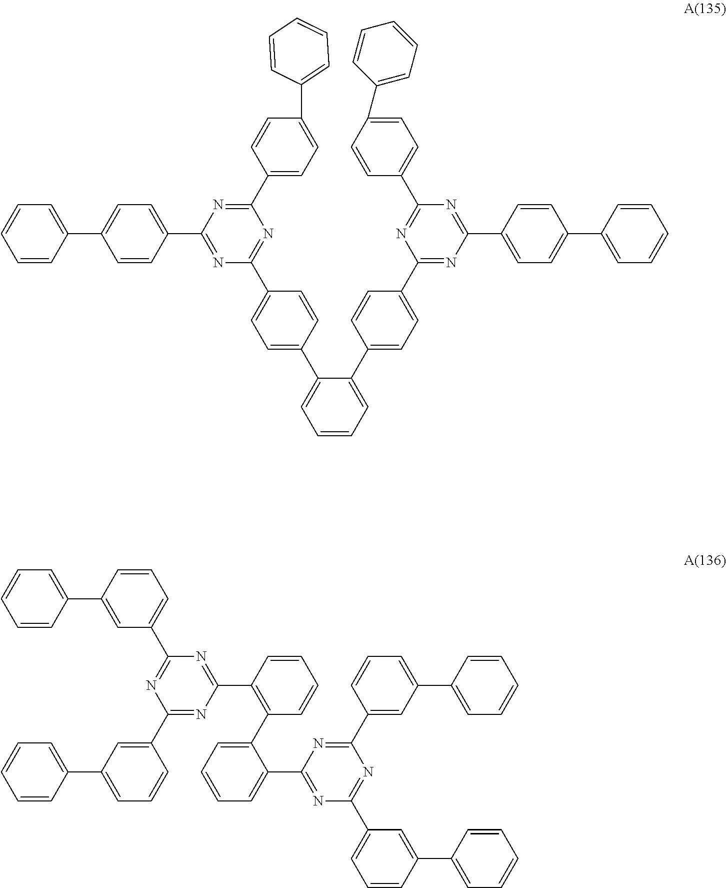 [SMILES: c1ccc(-c2ccc(-c3nc(-c4ccc(-c5ccccc5)cc4)nc(-c4ccc(-c5ccccc5-c5ccc(-c6nc(-c7ccc(-c8ccccc8)cc7)nc(-c7ccc(-c8ccccc8)cc7)n6)cc5)cc4)n3)cc2)cc1 ; c1ccc(-c2cccc(-c3nc(-c4cccc(-c5ccccc5)c4)nc(-c4ccccc4-c4ccccc4-c4nc(-c5cccc(-c6ccccc6)c5)nc(-c5cccc(-c6ccccc6)c5)n4)n3)c2)cc1]

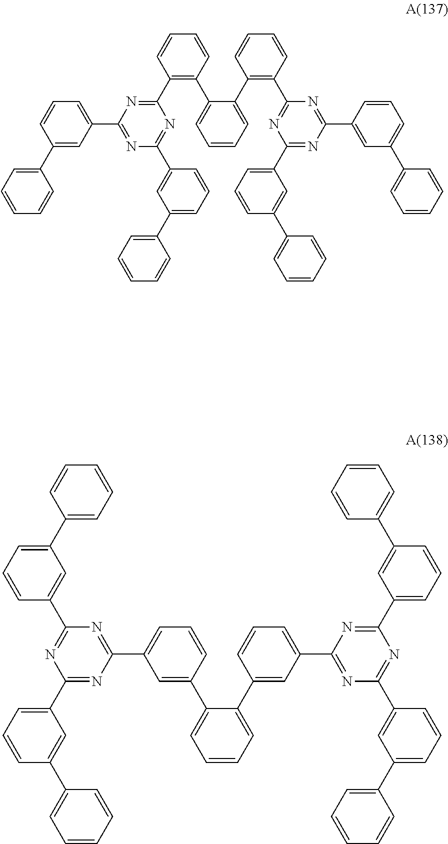 [SMILES: c1ccc(-c2cccc(-c3nc(-c4cccc(-c5ccccc5)c4)nc(-c4ccccc4-c4ccccc4-c4ccccc4-c4nc(-c5cccc(-c6ccccc6)c5)nc(-c5cccc(-c6ccccc6)c5)n4)n3)c2)cc1 ; c1ccc(-c2cccc(-c3nc(-c4cccc(-c5ccccc5)c4)nc(-c4cccc(-c5ccccc5-c5cccc(-c6nc(-c7cccc(-c8ccccc8)c7)nc(-c7cccc(-c8ccccc8)c7)n6)c5)c4)n3)c2)cc1]

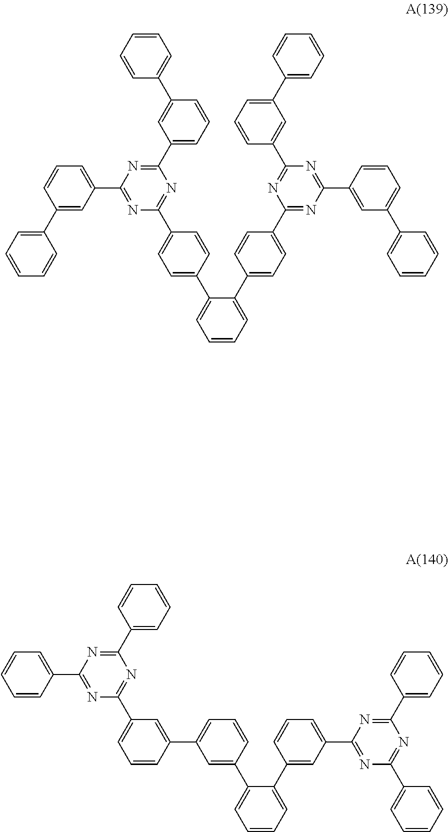 [SMILES: c1ccc(-c2cccc(-c3nc(-c4ccc(-c5ccccc5-c5ccc(-c6nc(-c7cccc(-c8ccccc8)c7)nc(-c7cccc(-c8ccccc8)c7)n6)cc5)cc4)nc(-c4cccc(-c5ccccc5)c4)n3)c2)cc1 ; c1ccc(-c2nc(-c3ccccc3)nc(-c3cccc(-c4cccc(-c5ccccc5-c5cccc(-c6nc(-c7ccccc7)nc(-c7ccccc7)n6)c5)c4)c3)n2)cc1]

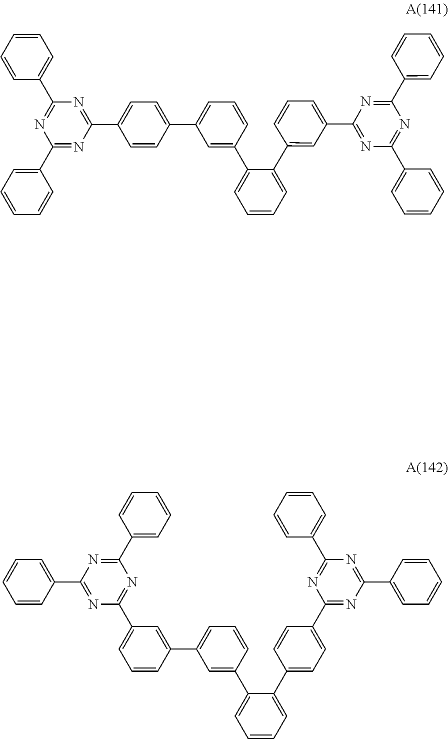 [SMILES: c1ccc(-c2nc(-c3ccccc3)nc(-c3ccc(-c4cccc(-c5ccccc5-c5cccc(-c6nc(-c7ccccc7)nc(-c7ccccc7)n6)c5)c4)cc3)n2)cc1 ; c1ccc(-c2nc(-c3ccccc3)nc(-c3ccc(-c4ccccc4-c4cccc(-c5cccc(-c6nc(-c7ccccc7)nc(-c7ccccc7)n6)c5)c4)cc3)n2)cc1]

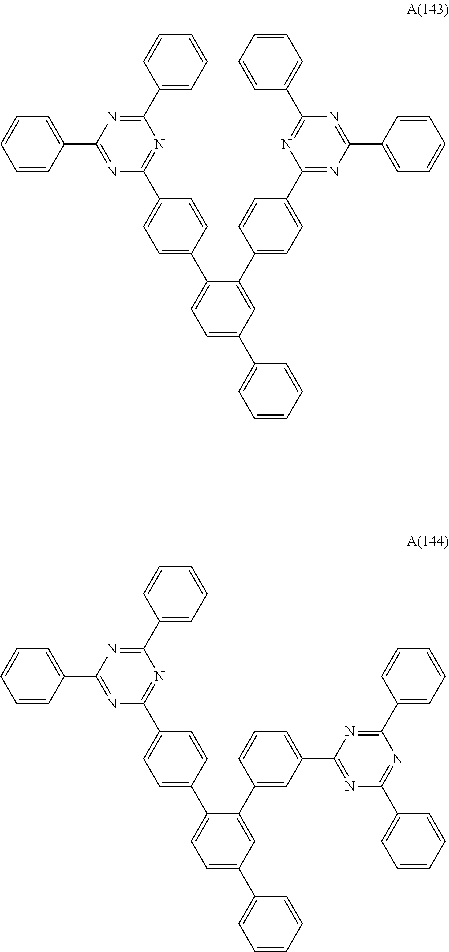 [SMILES: c1ccc(-c2ccc(-c3ccc(-c4nc(-c5ccccc5)nc(-c5ccccc5)n4)cc3)c(-c3ccc(-c4nc(-c5ccccc5)nc(-c5ccccc5)n4)cc3)c2)cc1 ; c1ccc(-c2ccc(-c3ccc(-c4nc(-c5ccccc5)nc(-c5ccccc5)n4)cc3)c(-c3cccc(-c4nc(-c5ccccc5)nc(-c5ccccc5)n4)c3)c2)cc1]

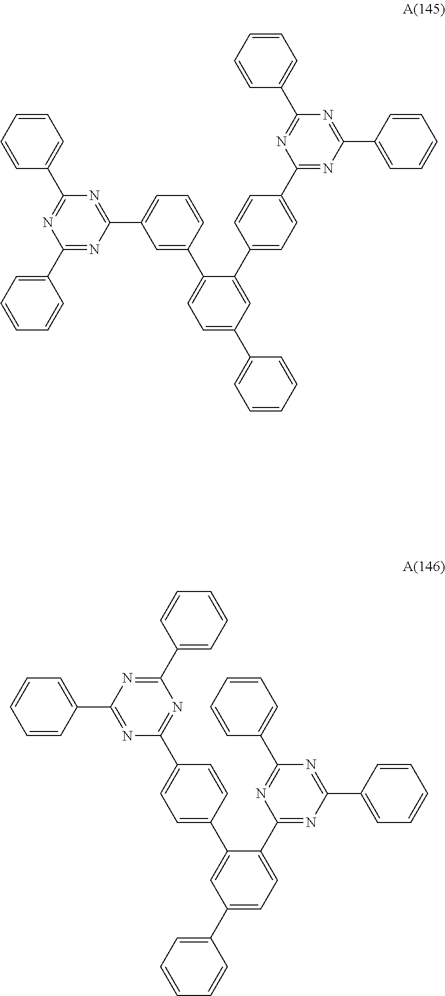 [SMILES: c1ccc(-c2ccc(-c3cccc(-c4nc(-c5ccccc5)nc(-c5ccccc5)n4)c3)c(-c3ccc(-c4nc(-c5ccccc5)nc(-c5ccccc5)n4)cc3)c2)cc1 ; c1ccc(-c2ccc(-c3nc(-c4ccccc4)nc(-c4ccccc4)n3)c(-c3ccc(-c4nc(-c5ccccc5)nc(-c5ccccc5)n4)cc3)c2)cc1]

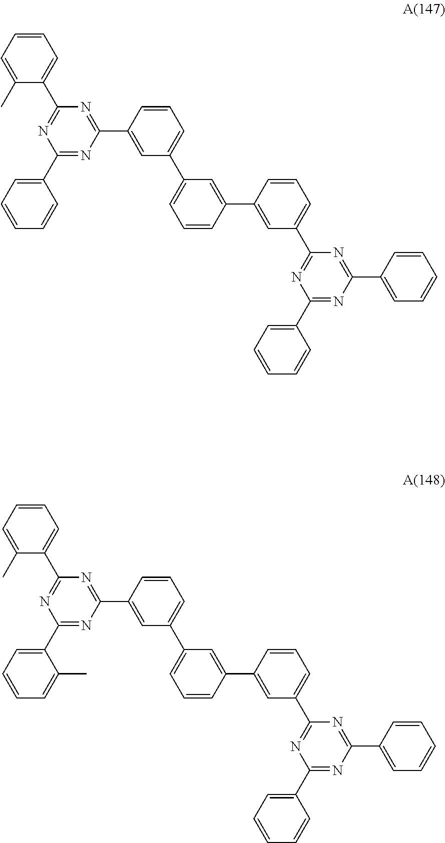 [SMILES: Cc1ccccc1-c1nc(-c2ccccc2)nc(-c2cccc(-c3cccc(-c4cccc(-c5nc(-c6ccccc6)nc(-c6ccccc6)n5)c4)c3)c2)n1 ; Cc1ccccc1-c1nc(-c2cccc(-c3cccc(-c4cccc(-c5nc(-c6ccccc6)nc(-c6ccccc6)n5)c4)c3)c2)nc(-c2ccccc2C)n1]

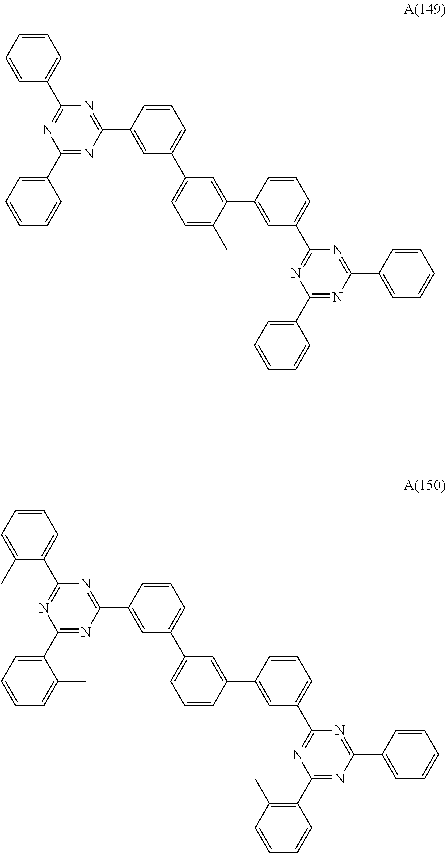 [SMILES: Cc1ccc(-c2cccc(-c3nc(-c4ccccc4)nc(-c4ccccc4)n3)c2)cc1-c1cccc(-c2nc(-c3ccccc3)nc(-c3ccccc3)n2)c1 ; Cc1ccccc1-c1nc(-c2ccccc2)nc(-c2cccc(-c3cccc(-c4cccc(-c5nc(-c6ccccc6C)nc(-c6ccccc6C)n5)c4)c3)c2)n1]

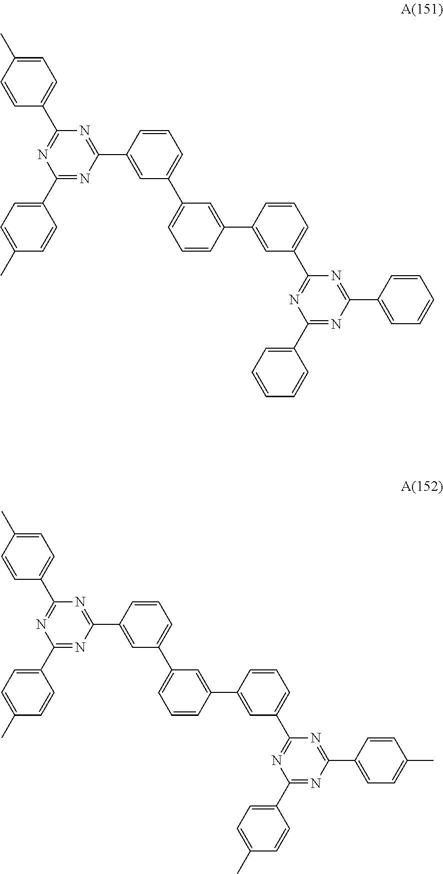 [SMILES: Cc1ccc(-c2nc(-c3ccc(C)cc3)nc(-c3cccc(-c4cccc(-c5cccc(-c6nc(-c7ccccc7)nc(-c7ccccc7)n6)c5)c4)c3)n2)cc1 ; Cc1ccc(-c2nc(-c3ccc(C)cc3)nc(-c3cccc(-c4cccc(-c5cccc(-c6nc(-c7ccc(C)cc7)nc(-c7ccc(C)cc7)n6)c5)c4)c3)n2)cc1]

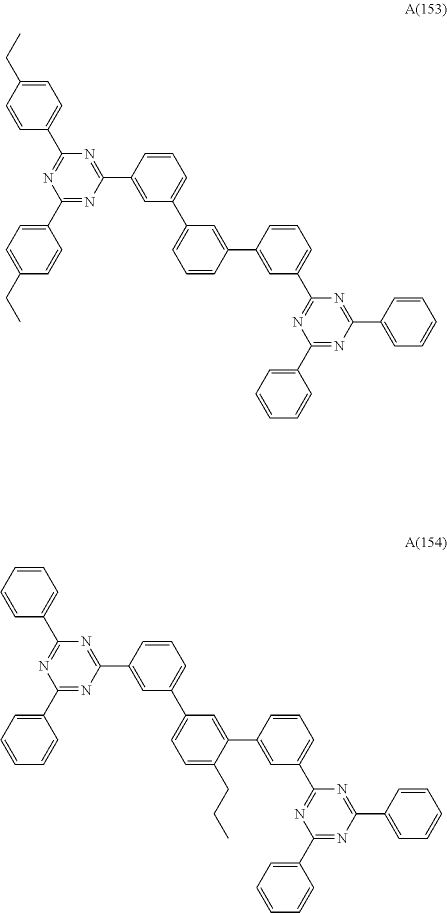 [SMILES: CCc1ccc(-c2nc(-c3ccc(CC)cc3)nc(-c3cccc(-c4cccc(-c5cccc(-c6nc(-c7ccccc7)nc(-c7ccccc7)n6)c5)c4)c3)n2)cc1 ; CCCc1ccc(-c2cccc(-c3nc(-c4ccccc4)nc(-c4ccccc4)n3)c2)cc1-c1cccc(-c2nc(-c3ccccc3)nc(-c3ccccc3)n2)c1]

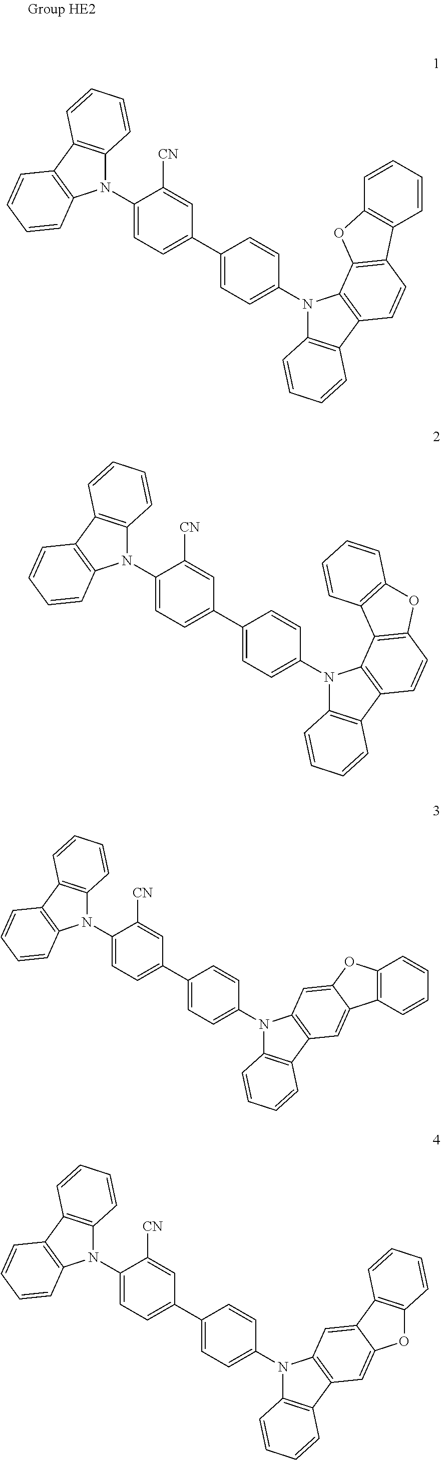 [SMILES: N#Cc1cc(-c2ccc(-n3c4ccccc4c4ccc5c6ccccc6oc5c43)cc2)ccc1-n1c2ccccc2c2ccccc21 ; N#Cc1cc(-c2ccc(-n3c4ccccc4c4ccc5oc6ccccc6c5c43)cc2)ccc1-n1c2ccccc2c2ccccc21 ; N#Cc1cc(-c2ccc(-n3c4ccccc4c4cc5c(cc43)oc3ccccc35)cc2)ccc1-n1c2ccccc2c2ccccc21 ; N#Cc1cc(-c2ccc(-n3c4ccccc4c4cc5oc6ccccc6c5cc43)cc2)ccc1-n1c2ccccc2c2ccccc21]

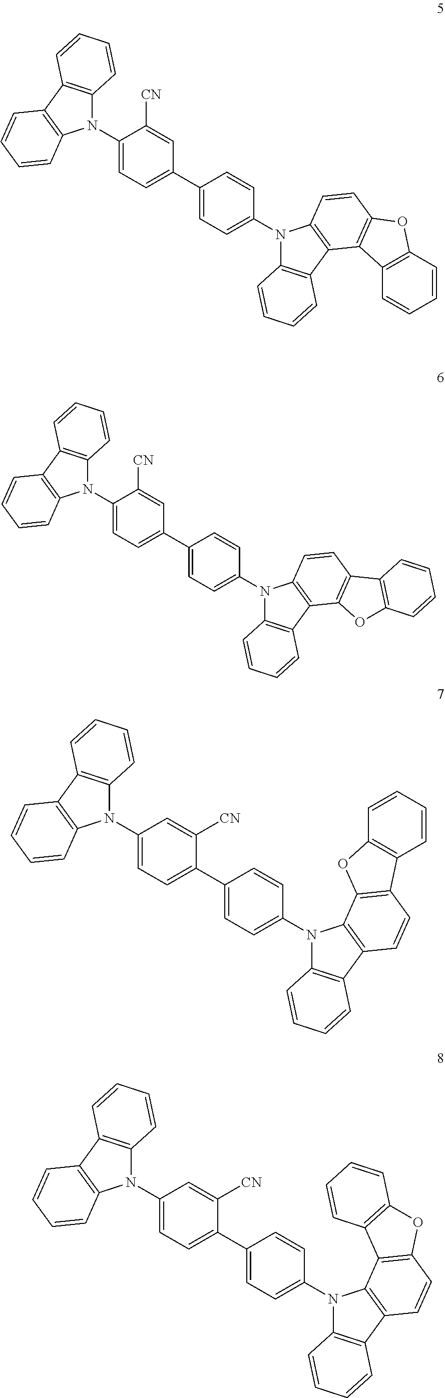 [SMILES: N#Cc1cc(-c2ccc(-n3c4ccccc4c4c5c(ccc43)oc3ccccc35)cc2)ccc1-n1c2ccccc2c2ccccc21 ; N#Cc1cc(-c2ccc(-n3c4ccccc4c4c5oc6ccccc6c5ccc43)cc2)ccc1-n1c2ccccc2c2ccccc21 ; N#Cc1cc(-n2c3ccccc3c3ccccc32)ccc1-c1ccc(-n2c3ccccc3c3ccc4c5ccccc5oc4c32)cc1 ; N#Cc1cc(-n2c3ccccc3c3ccccc32)ccc1-c1ccc(-n2c3ccccc3c3ccc4oc5ccccc5c4c32)cc1]

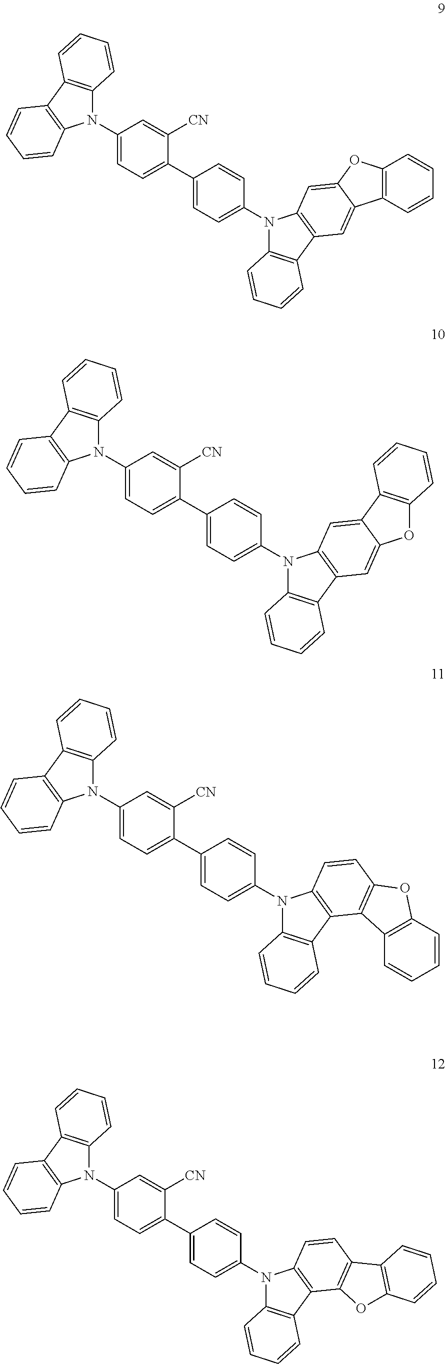 [SMILES: N#Cc1cc(-n2c3ccccc3c3ccccc32)ccc1-c1ccc(-n2c3ccccc3c3cc4c(cc32)oc2ccccc24)cc1 ; N#Cc1cc(-n2c3ccccc3c3ccccc32)ccc1-c1ccc(-n2c3ccccc3c3cc4oc5ccccc5c4cc32)cc1 ; N#Cc1cc(-n2c3ccccc3c3ccccc32)ccc1-c1ccc(-n2c3ccccc3c3c4c(ccc32)oc2ccccc24)cc1 ; N#Cc1cc(-n2c3ccccc3c3ccccc32)ccc1-c1ccc(-n2c3ccccc3c3c4oc5ccccc5c4ccc32)cc1]

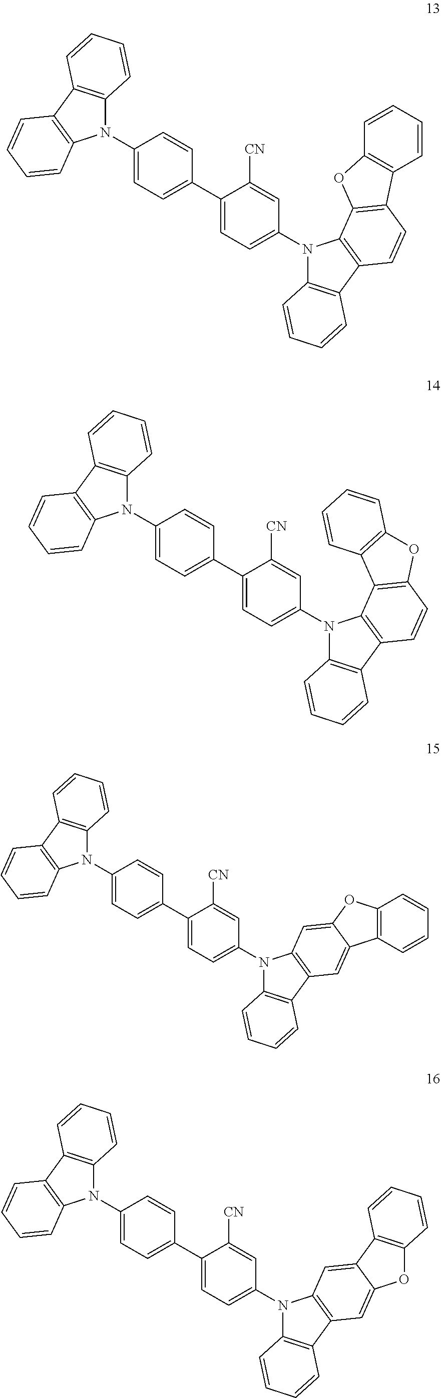 [SMILES: N#Cc1cc(-n2c3ccccc3c3ccc4c5ccccc5oc4c32)ccc1-c1ccc(-n2c3ccccc3c3ccccc32)cc1 ; N#Cc1cc(-n2c3ccccc3c3ccc4oc5ccccc5c4c32)ccc1-c1ccc(-n2c3ccccc3c3ccccc32)cc1 ; N#Cc1cc(-n2c3ccccc3c3cc4c(cc32)oc2ccccc24)ccc1-c1ccc(-n2c3ccccc3c3ccccc32)cc1 ; N#Cc1cc(-n2c3ccccc3c3cc4oc5ccccc5c4cc32)ccc1-c1ccc(-n2c3ccccc3c3ccccc32)cc1]

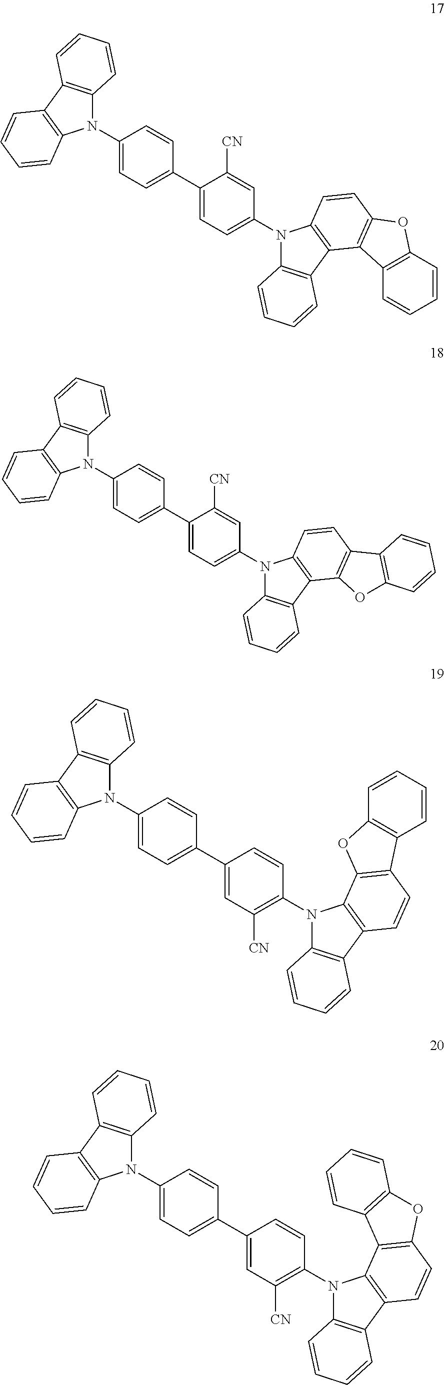 [SMILES: N#Cc1cc(-n2c3ccccc3c3c4c(ccc32)oc2ccccc24)ccc1-c1ccc(-n2c3ccccc3c3ccccc32)cc1 ; N#Cc1cc(-n2c3ccccc3c3c4oc5ccccc5c4ccc32)ccc1-c1ccc(-n2c3ccccc3c3ccccc32)cc1 ; N#Cc1cc(-c2ccc(-n3c4ccccc4c4ccccc43)cc2)ccc1-n1c2ccccc2c2ccc3c4ccccc4oc3c21 ; N#Cc1cc(-c2ccc(-n3c4ccccc4c4ccccc43)cc2)ccc1-n1c2ccccc2c2ccc3oc4ccccc4c3c21]

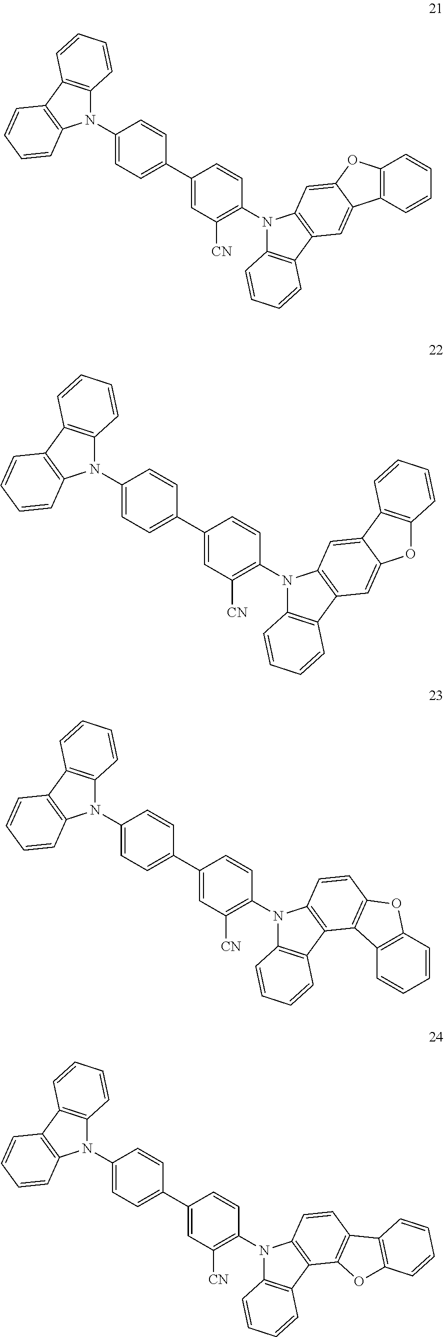 [SMILES: N#Cc1cc(-c2ccc(-n3c4ccccc4c4ccccc43)cc2)ccc1-n1c2ccccc2c2cc3c(cc21)oc1ccccc13 ; N#Cc1cc(-c2ccc(-n3c4ccccc4c4ccccc43)cc2)ccc1-n1c2ccccc2c2cc3oc4ccccc4c3cc21 ; N#Cc1cc(-c2ccc(-n3c4ccccc4c4ccccc43)cc2)ccc1-n1c2ccccc2c2c3c(ccc21)oc1ccccc13 ; N#Cc1cc(-c2ccc(-n3c4ccccc4c4ccccc43)cc2)ccc1-n1c2ccccc2c2c3oc4ccccc4c3ccc21]

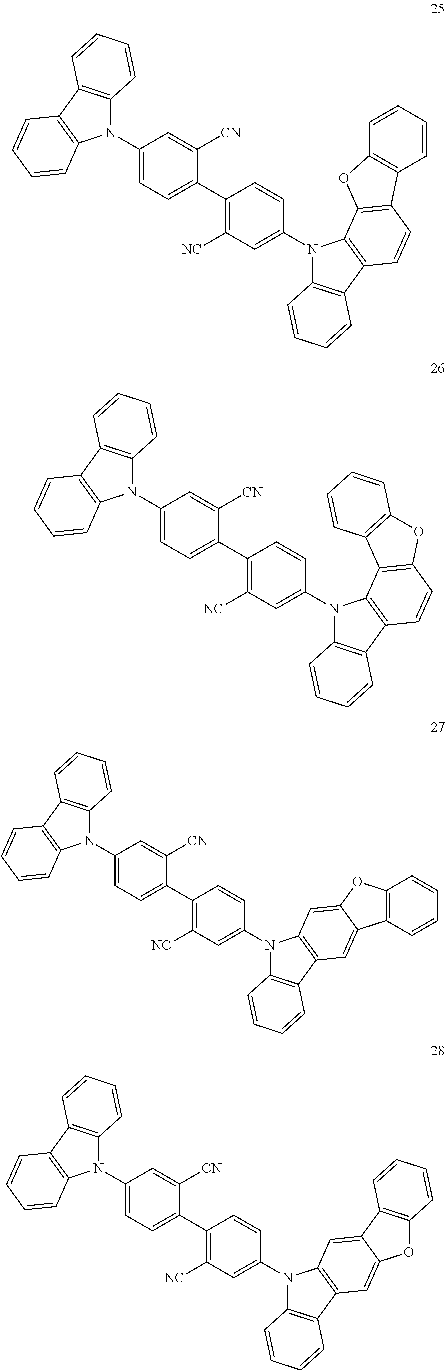 [SMILES: N#Cc1cc(-n2c3ccccc3c3ccccc32)ccc1-c1ccc(-n2c3ccccc3c3ccc4c5ccccc5oc4c32)cc1C#N ; N#Cc1cc(-n2c3ccccc3c3ccccc32)ccc1-c1ccc(-n2c3ccccc3c3ccc4oc5ccccc5c4c32)cc1C#N ; N#Cc1cc(-n2c3ccccc3c3ccccc32)ccc1-c1ccc(-n2c3ccccc3c3cc4c(cc32)oc2ccccc24)cc1C#N ; N#Cc1cc(-n2c3ccccc3c3ccccc32)ccc1-c1ccc(-n2c3ccccc3c3cc4oc5ccccc5c4cc32)cc1C#N]

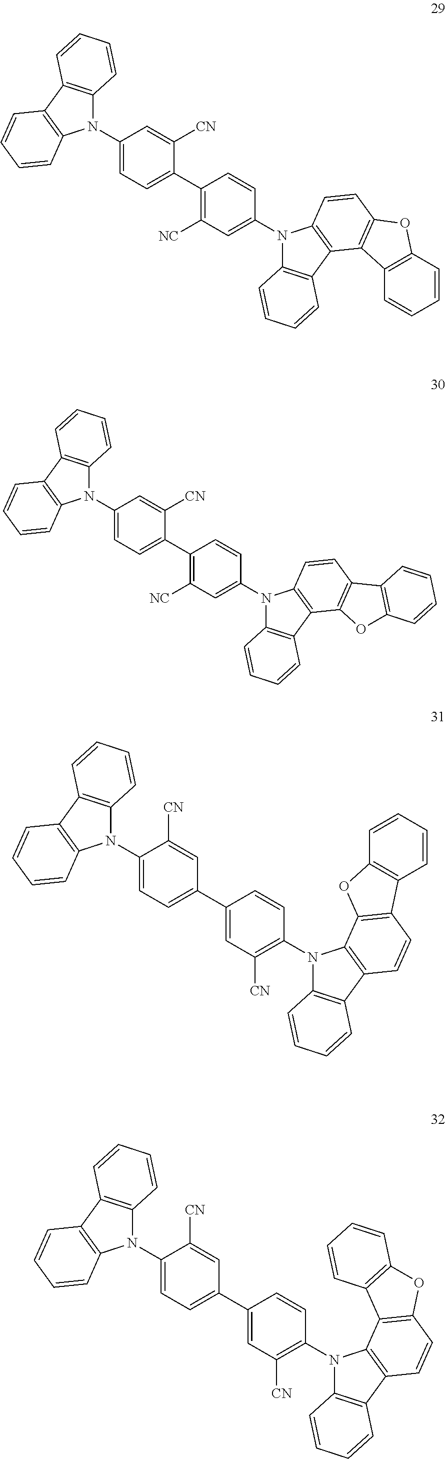 [SMILES: N#Cc1cc(-n2c3ccccc3c3ccccc32)ccc1-c1ccc(-n2c3ccccc3c3c4c(ccc32)oc2ccccc24)cc1C#N ; N#Cc1cc(-n2c3ccccc3c3ccccc32)ccc1-c1ccc(-n2c3ccccc3c3c4oc5ccccc5c4ccc32)cc1C#N ; N#Cc1cc(-c2ccc(-n3c4ccccc4c4ccc5c6ccccc6oc5c43)c(C#N)c2)ccc1-n1c2ccccc2c2ccccc21 ; N#Cc1cc(-c2ccc(-n3c4ccccc4c4ccc5oc6ccccc6c5c43)c(C#N)c2)ccc1-n1c2ccccc2c2ccccc21]

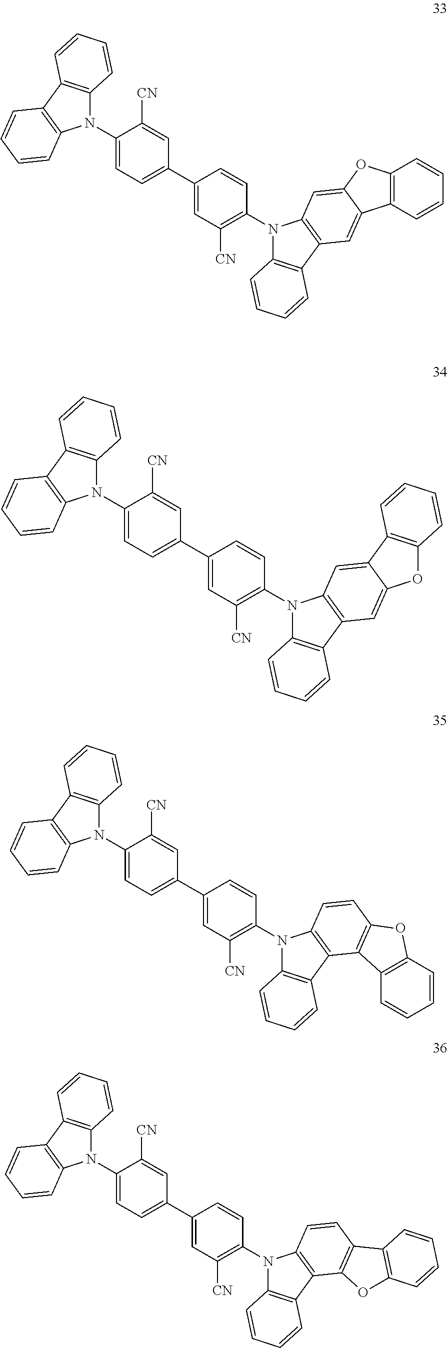 [SMILES: N#Cc1cc(-c2ccc(-n3c4ccccc4c4cc5c(cc43)oc3ccccc35)c(C#N)c2)ccc1-n1c2ccccc2c2ccccc21 ; N#Cc1cc(-c2ccc(-n3c4ccccc4c4cc5oc6ccccc6c5cc43)c(C#N)c2)ccc1-n1c2ccccc2c2ccccc21 ; N#Cc1cc(-c2ccc(-n3c4ccccc4c4c5c(ccc43)oc3ccccc35)c(C#N)c2)ccc1-n1c2ccccc2c2ccccc21 ; N#Cc1cc(-c2ccc(-n3c4ccccc4c4c5oc6ccccc6c5ccc43)c(C#N)c2)ccc1-n1c2ccccc2c2ccccc21]

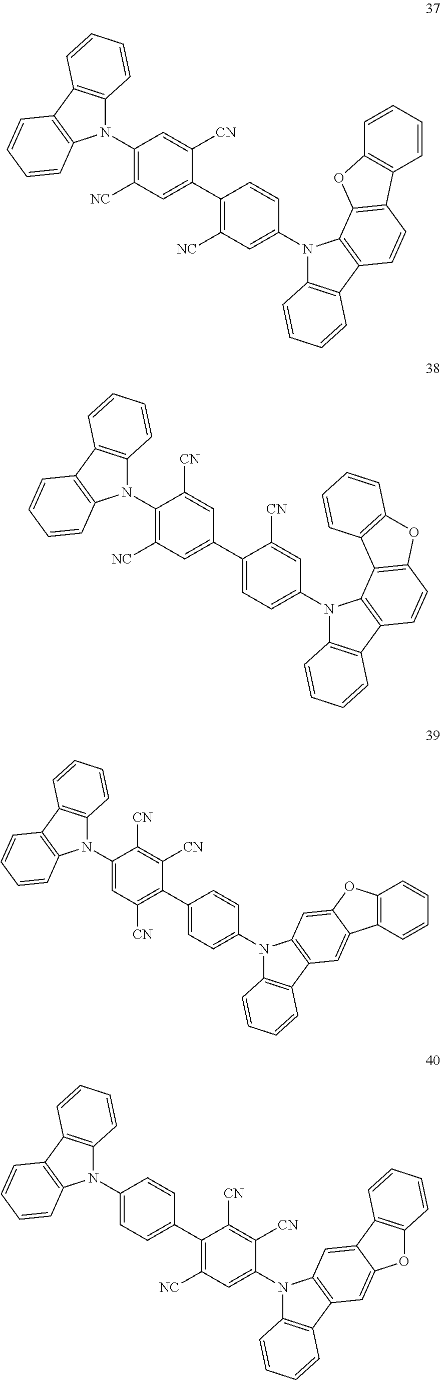 [SMILES: N#Cc1cc(-n2c3ccccc3c3ccc4c5ccccc5oc4c32)ccc1-c1cc(C#N)c(-n2c3ccccc3c3ccccc32)cc1C#N ; N#Cc1cc(-n2c3ccccc3c3ccc4oc5ccccc5c4c32)ccc1-c1cc(C#N)c(-n2c3ccccc3c3ccccc32)c(C#N)c1 ; N#Cc1cc(-n2c3ccccc3c3ccccc32)c(C#N)c(C#N)c1-c1ccc(-n2c3ccccc3c3cc4c(cc32)oc2ccccc24)cc1 ; N#Cc1cc(-n2c3ccccc3c3cc4oc5ccccc5c4cc32)c(C#N)c(C#N)c1-c1ccc(-n2c3ccccc3c3ccccc32)cc1]

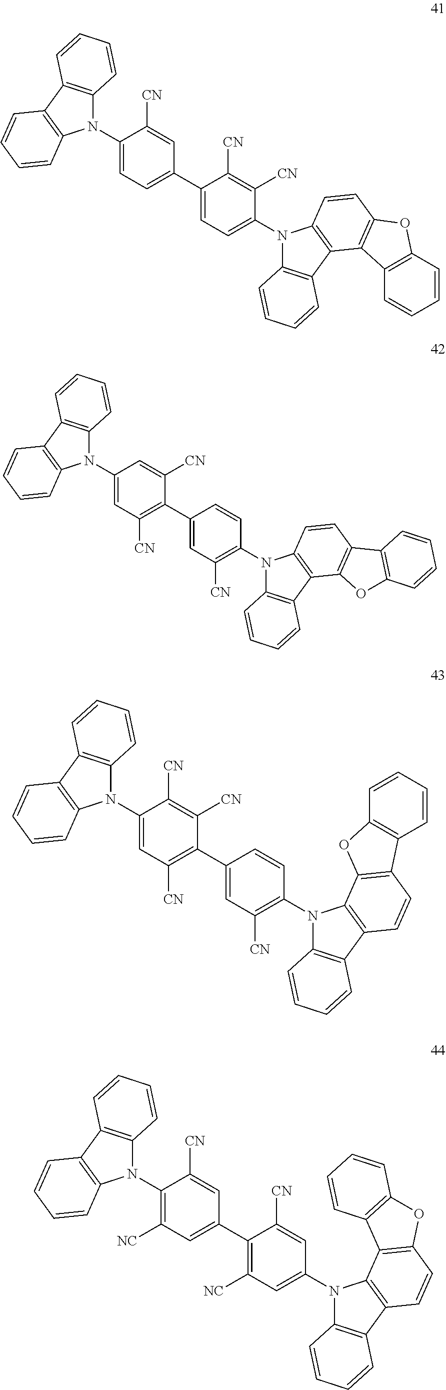 [SMILES: N#Cc1cc(-c2ccc(-n3c4ccccc4c4c5c(ccc43)oc3ccccc35)c(C#N)c2C#N)ccc1-n1c2ccccc2c2ccccc21 ; N#Cc1cc(-c2c(C#N)cc(-n3c4ccccc4c4ccccc43)cc2C#N)ccc1-n1c2ccccc2c2c3oc4ccccc4c3ccc21 ; N#Cc1cc(-c2c(C#N)cc(-n3c4ccccc4c4ccccc43)c(C#N)c2C#N)ccc1-n1c2ccccc2c2ccc3c4ccccc4oc3c21 ; N#Cc1cc(-n2c3ccccc3c3ccc4oc5ccccc5c4c32)cc(C#N)c1-c1cc(C#N)c(-n2c3ccccc3c3ccccc32)c(C#N)c1]

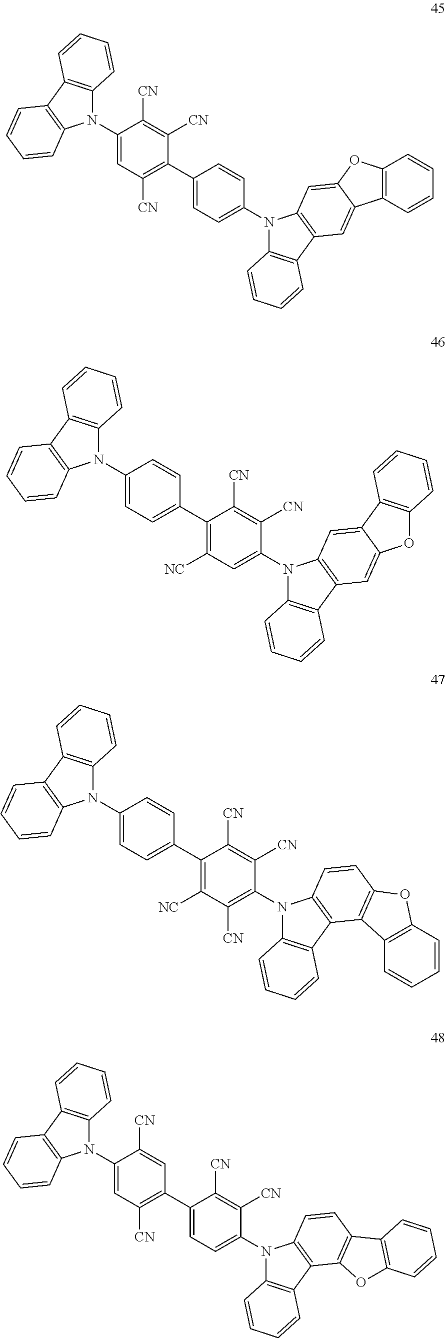 [SMILES: N#Cc1cc(-n2c3ccccc3c3ccccc32)c(C#N)c(C#N)c1-c1ccc(-n2c3ccccc3c3cc4c(cc32)oc2ccccc24)cc1 ; N#Cc1cc(-n2c3ccccc3c3cc4oc5ccccc5c4cc32)c(C#N)c(C#N)c1-c1ccc(-n2c3ccccc3c3ccccc32)cc1 ; N#Cc1c(C#N)c(-n2c3ccccc3c3c4c(ccc32)oc2ccccc24)c(C#N)c(C#N)c1-c1ccc(-n2c3ccccc3c3ccccc32)cc1 ; N#Cc1cc(-n2c3ccccc3c3ccccc32)c(C#N)cc1-c1ccc(-n2c3ccccc3c3c4oc5ccccc5c4ccc32)c(C#N)c1C#N]

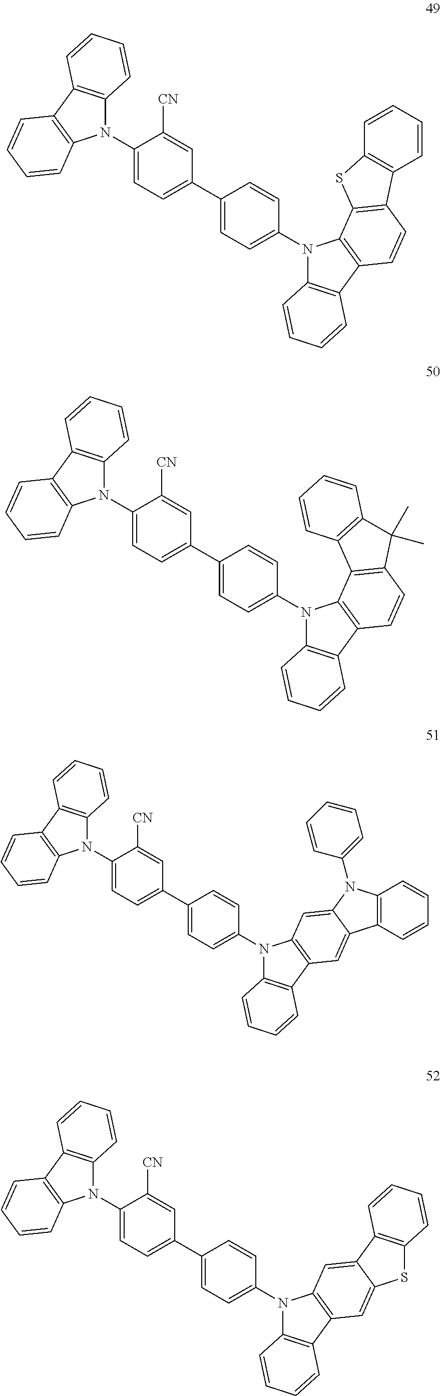 [SMILES: N#Cc1cc(-c2ccc(-n3c4ccccc4c4ccc5c6ccccc6sc5c43)cc2)ccc1-n1c2ccccc2c2ccccc21 ; CC1(C)c2ccccc2-c2c1ccc1c3ccccc3n(-c3ccc(-c4ccc(-n5c6ccccc6c6ccccc65)c(C#N)c4)cc3)c21 ; N#Cc1cc(-c2ccc(-n3c4ccccc4c4cc5c6ccccc6n(-c6ccccc6)c5cc43)cc2)ccc1-n1c2ccccc2c2ccccc21 ; N#Cc1cc(-c2ccc(-n3c4ccccc4c4cc5sc6ccccc6c5cc43)cc2)ccc1-n1c2ccccc2c2ccccc21]

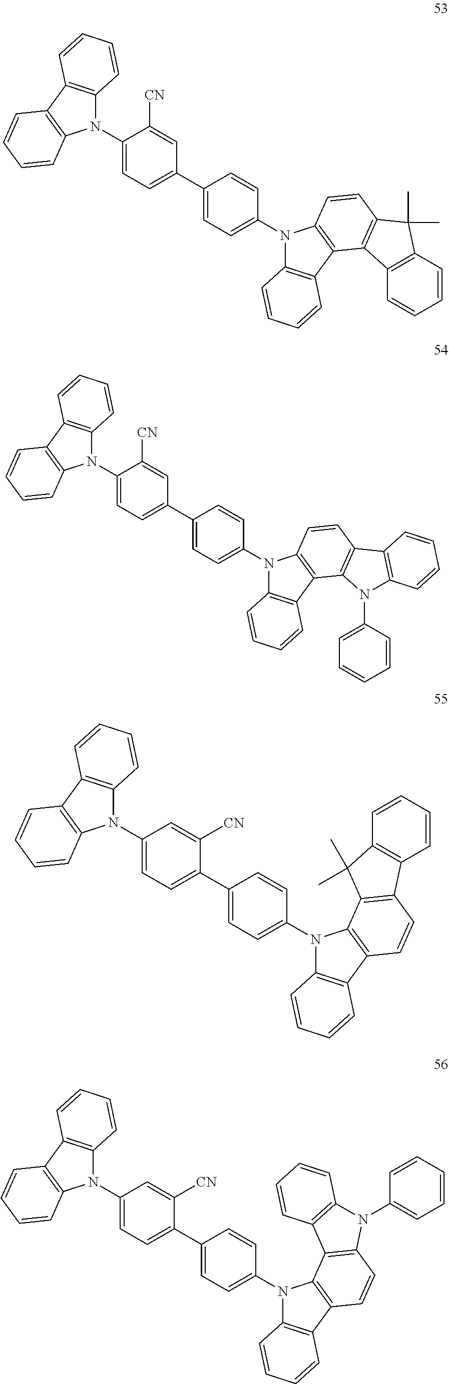 [SMILES: CC1(C)c2ccccc2-c2c1ccc1c2c2ccccc2n1-c1ccc(-c2ccc(-n3c4ccccc4c4ccccc43)c(C#N)c2)cc1 ; N#Cc1cc(-c2ccc(-n3c4ccccc4c4c3ccc3c5ccccc5n(-c5ccccc5)c34)cc2)ccc1-n1c2ccccc2c2ccccc21 ; CC1(C)c2ccccc2-c2ccc3c4ccccc4n(-c4ccc(-c5ccc(-n6c7ccccc7c7ccccc76)cc5C#N)cc4)c3c21 ; N#Cc1cc(-n2c3ccccc3c3ccccc32)ccc1-c1ccc(-n2c3ccccc3c3ccc4c(c5ccccc5n4-c4ccccc4)c32)cc1]

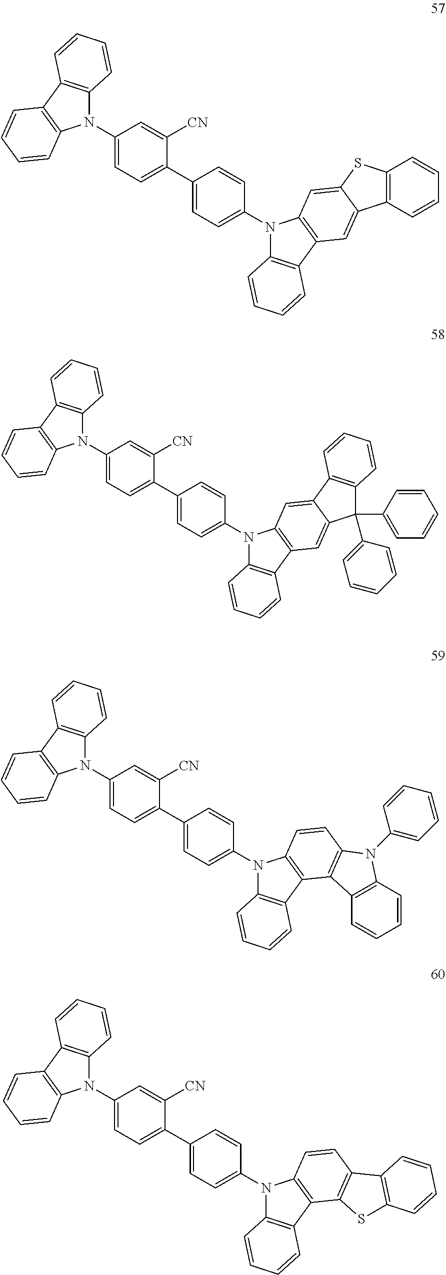 [SMILES: N#Cc1cc(-n2c3ccccc3c3ccccc32)ccc1-c1ccc(-n2c3ccccc3c3cc4c(cc32)sc2ccccc24)cc1 ; N#Cc1cc(-n2c3ccccc3c3ccccc32)ccc1-c1ccc(-n2c3ccccc3c3cc4c(cc32)-c2ccccc2C4(c2ccccc2)c2ccccc2)cc1 ; N#Cc1cc(-n2c3ccccc3c3ccccc32)ccc1-c1ccc(-n2c3ccccc3c3c4c5ccccc5n(-c5ccccc5)c4ccc32)cc1 ; N#Cc1cc(-n2c3ccccc3c3ccccc32)ccc1-c1ccc(-n2c3ccccc3c3c4sc5ccccc5c4ccc32)cc1]

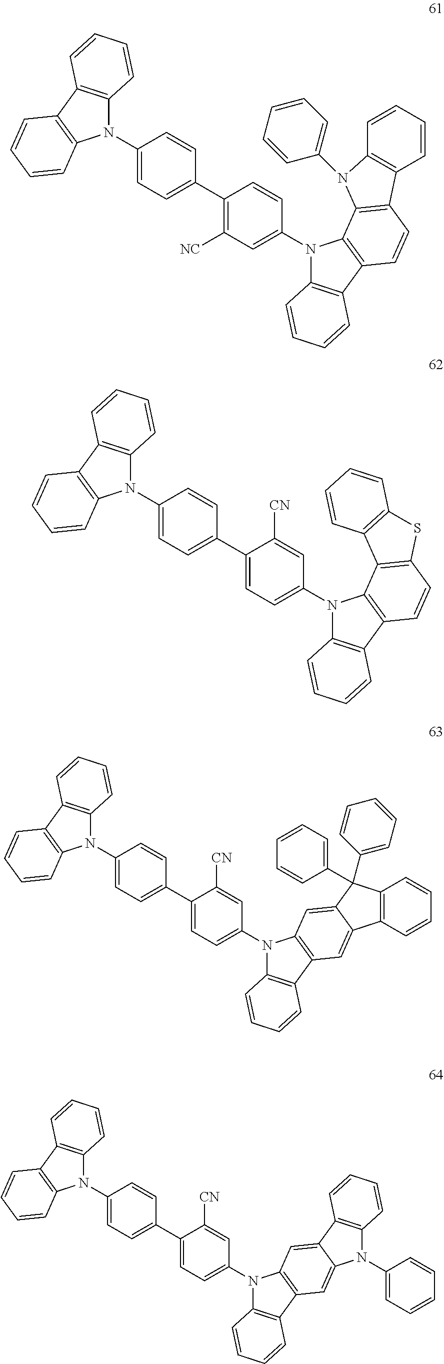 [SMILES: N#Cc1cc(-n2c3ccccc3c3ccc4c5ccccc5n(-c5ccccc5)c4c32)ccc1-c1ccc(-n2c3ccccc3c3ccccc32)cc1 ; N#Cc1cc(-n2c3ccccc3c3ccc4sc5ccccc5c4c32)ccc1-c1ccc(-n2c3ccccc3c3ccccc32)cc1 ; N#Cc1cc(-n2c3ccccc3c3cc4c(cc32)C(c2ccccc2)(c2ccccc2)c2ccccc2-4)ccc1-c1ccc(-n2c3ccccc3c3ccccc32)cc1 ; N#Cc1cc(-n2c3ccccc3c3cc4c(cc32)c2ccccc2n4-c2ccccc2)ccc1-c1ccc(-n2c3ccccc3c3ccccc32)cc1]

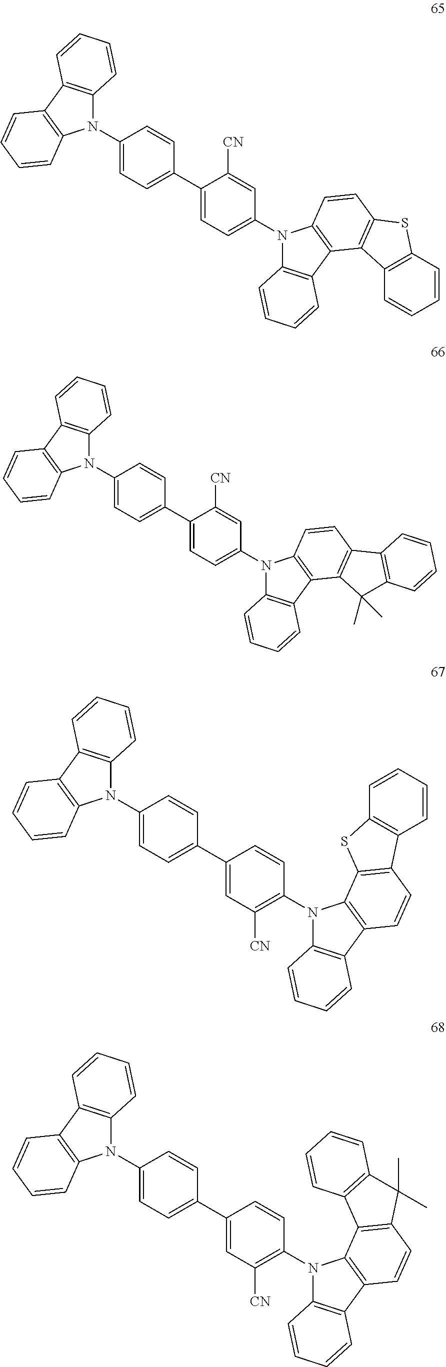 [SMILES: N#Cc1cc(-n2c3ccccc3c3c4c(ccc32)sc2ccccc24)ccc1-c1ccc(-n2c3ccccc3c3ccccc32)cc1 ; CC1(C)c2ccccc2-c2ccc3c(c21)c1ccccc1n3-c1ccc(-c2ccc(-n3c4ccccc4c4ccccc43)cc2)c(C#N)c1 ; N#Cc1cc(-c2ccc(-n3c4ccccc4c4ccccc43)cc2)ccc1-n1c2ccccc2c2ccc3c4ccccc4sc3c21 ; CC1(C)c2ccccc2-c2c1ccc1c3ccccc3n(-c3ccc(-c4ccc(-n5c6ccccc6c6ccccc65)cc4)cc3C#N)c21]

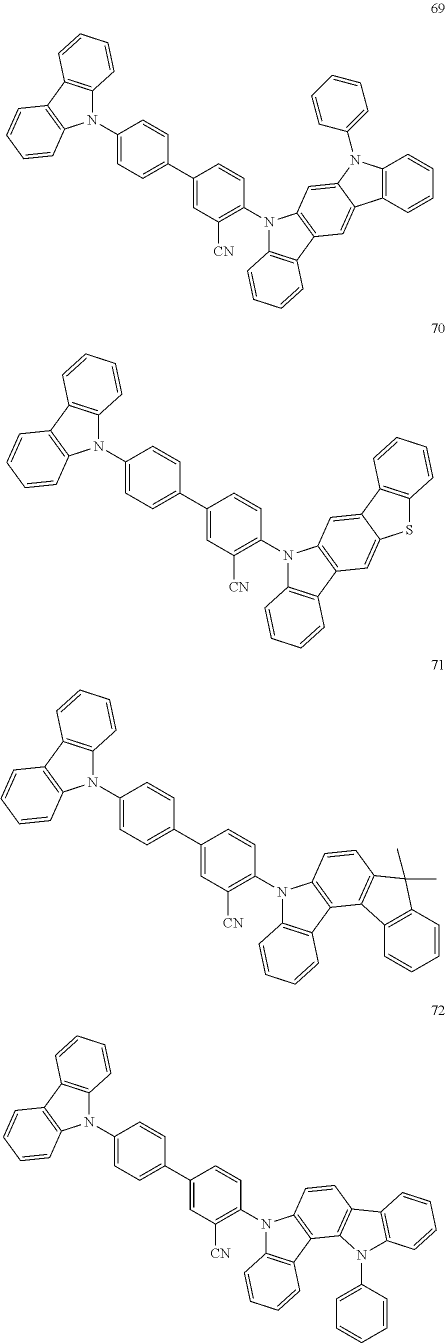 [SMILES: N#Cc1cc(-c2ccc(-n3c4ccccc4c4ccccc43)cc2)ccc1-n1c2ccccc2c2cc3c4ccccc4n(-c4ccccc4)c3cc21 ; N#Cc1cc(-c2ccc(-n3c4ccccc4c4ccccc43)cc2)ccc1-n1c2ccccc2c2cc3sc4ccccc4c3cc21 ; CC1(C)c2ccccc2-c2c1ccc1c2c2ccccc2n1-c1ccc(-c2ccc(-n3c4ccccc4c4ccccc43)cc2)cc1C#N ; N#Cc1cc(-c2ccc(-n3c4ccccc4c4ccccc43)cc2)ccc1-n1c2ccccc2c2c1ccc1c3ccccc3n(-c3ccccc3)c12]

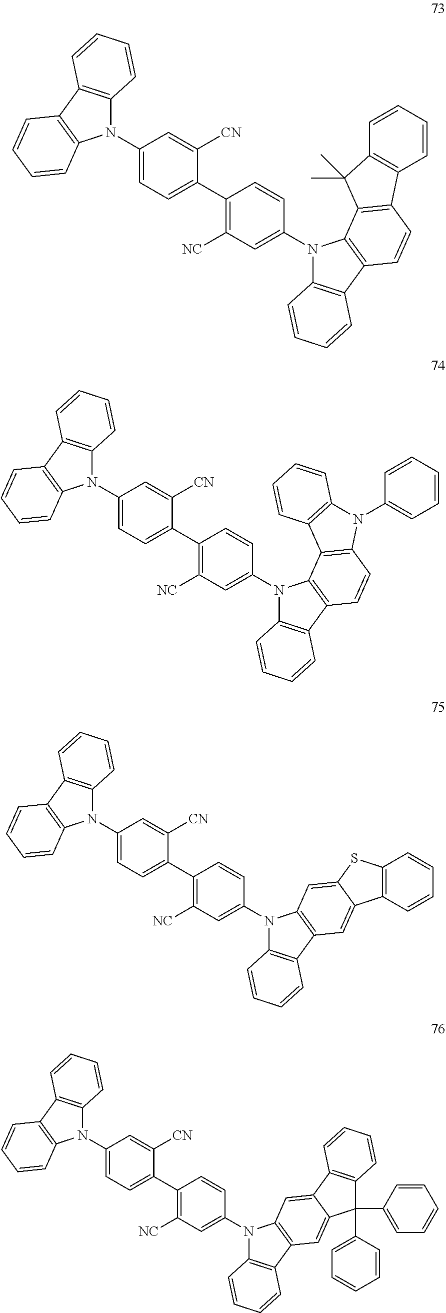 [SMILES: CC1(C)c2ccccc2-c2ccc3c4ccccc4n(-c4ccc(-c5ccc(-n6c7ccccc7c7ccccc76)cc5C#N)c(C#N)c4)c3c21 ; N#Cc1cc(-n2c3ccccc3c3ccccc32)ccc1-c1ccc(-n2c3ccccc3c3ccc4c(c5ccccc5n4-c4ccccc4)c32)cc1C#N ; N#Cc1cc(-n2c3ccccc3c3ccccc32)ccc1-c1ccc(-n2c3ccccc3c3cc4c(cc32)sc2ccccc24)cc1C#N ; N#Cc1cc(-n2c3ccccc3c3ccccc32)ccc1-c1ccc(-n2c3ccccc3c3cc4c(cc32)-c2ccccc2C4(c2ccccc2)c2ccccc2)cc1C#N]

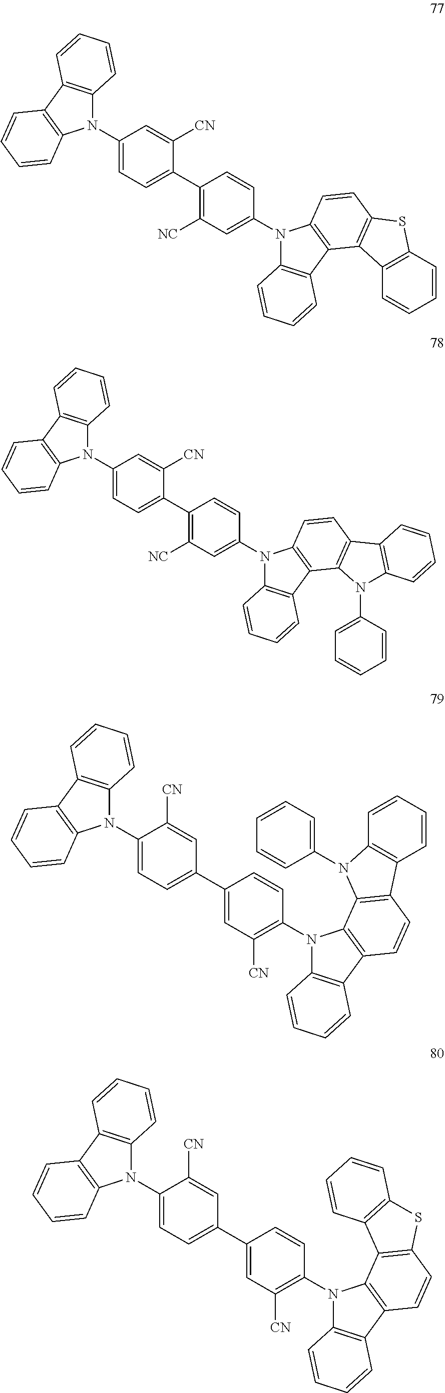 [SMILES: N#Cc1cc(-n2c3ccccc3c3ccccc32)ccc1-c1ccc(-n2c3ccccc3c3c4c(ccc32)sc2ccccc24)cc1C#N ; N#Cc1cc(-n2c3ccccc3c3ccccc32)ccc1-c1ccc(-n2c3ccccc3c3c2ccc2c4ccccc4n(-c4ccccc4)c23)cc1C#N ; N#Cc1cc(-c2ccc(-n3c4ccccc4c4ccc5c6ccccc6n(-c6ccccc6)c5c43)c(C#N)c2)ccc1-n1c2ccccc2c2ccccc21 ; N#Cc1cc(-c2ccc(-n3c4ccccc4c4ccc5sc6ccccc6c5c43)c(C#N)c2)ccc1-n1c2ccccc2c2ccccc21]

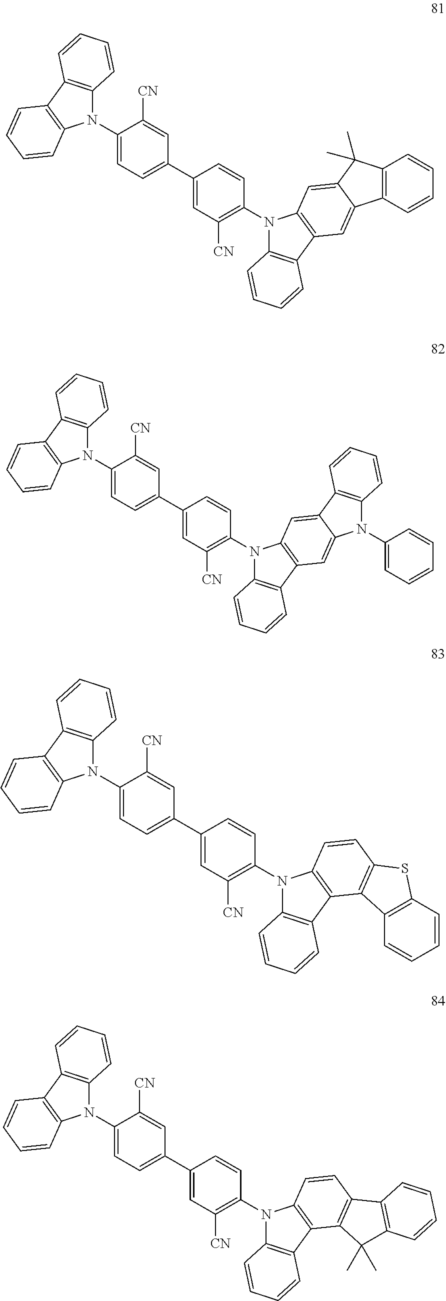 [SMILES: CC1(C)c2ccccc2-c2cc3c4ccccc4n(-c4ccc(-c5ccc(-n6c7ccccc7c7ccccc76)c(C#N)c5)cc4C#N)c3cc21 ; N#Cc1cc(-c2ccc(-n3c4ccccc4c4cc5c(cc43)c3ccccc3n5-c3ccccc3)c(C#N)c2)ccc1-n1c2ccccc2c2ccccc21 ; N#Cc1cc(-c2ccc(-n3c4ccccc4c4c5c(ccc43)sc3ccccc35)c(C#N)c2)ccc1-n1c2ccccc2c2ccccc21 ; CC1(C)c2ccccc2-c2ccc3c(c21)c1ccccc1n3-c1ccc(-c2ccc(-n3c4ccccc4c4ccccc43)c(C#N)c2)cc1C#N]

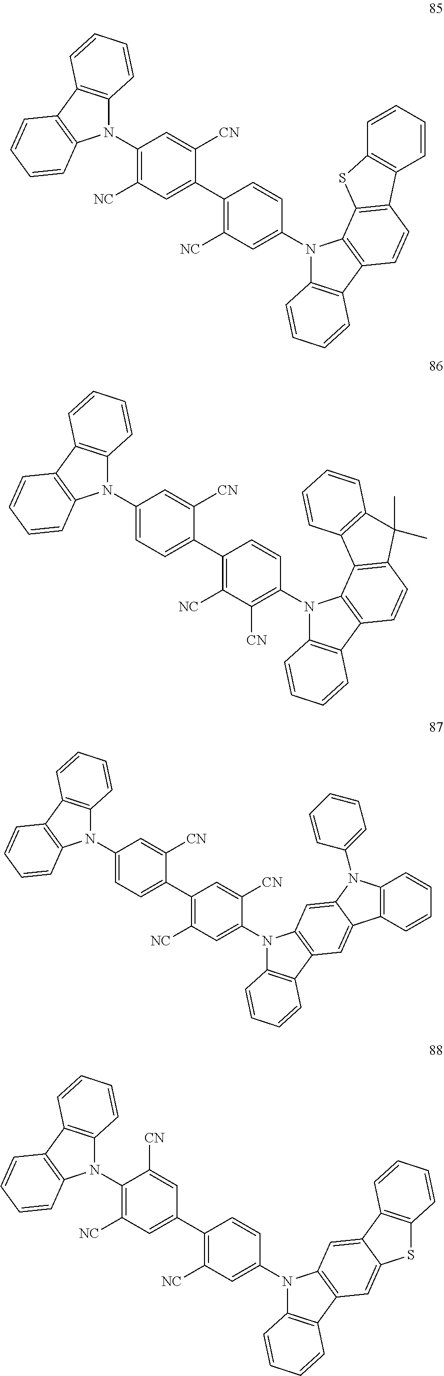 [SMILES: N#Cc1cc(-n2c3ccccc3c3ccc4c5ccccc5sc4c32)ccc1-c1cc(C#N)c(-n2c3ccccc3c3ccccc32)cc1C#N ; CC1(C)c2ccccc2-c2c1ccc1c3ccccc3n(-c3ccc(-c4ccc(-n5c6ccccc6c6ccccc65)cc4C#N)c(C#N)c3C#N)c21 ; N#Cc1cc(-n2c3ccccc3c3ccccc32)ccc1-c1cc(C#N)c(-n2c3ccccc3c3cc4c5ccccc5n(-c5ccccc5)c4cc32)cc1C#N ; N#Cc1cc(-n2c3ccccc3c3cc4sc5ccccc5c4cc32)ccc1-c1cc(C#N)c(-n2c3ccccc3c3ccccc32)c(C#N)c1]

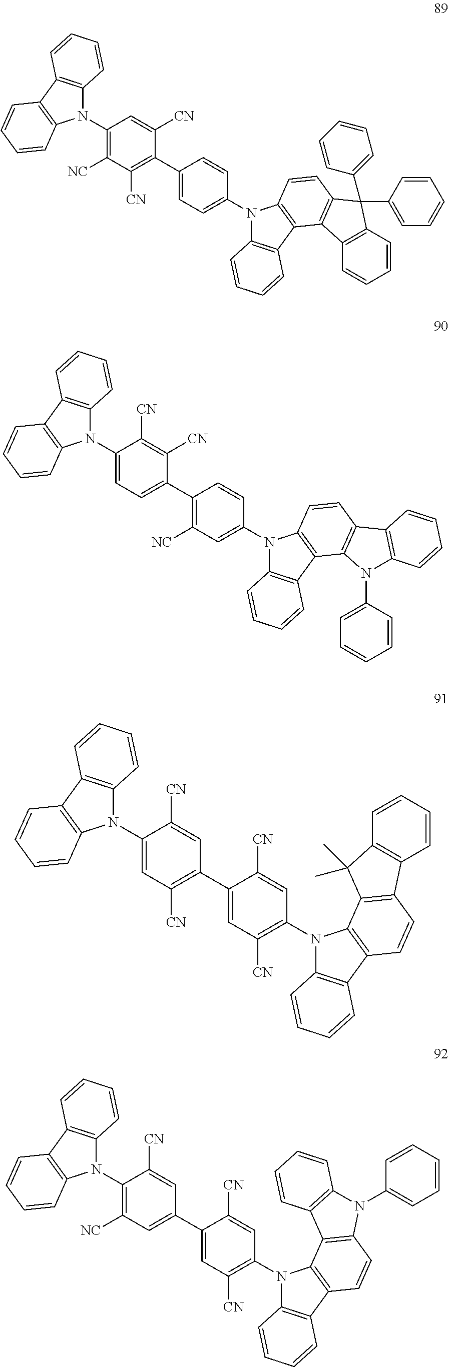 [SMILES: N#Cc1cc(-n2c3ccccc3c3ccccc32)c(C#N)c(C#N)c1-c1ccc(-n2c3ccccc3c3c4c(ccc32)C(c2ccccc2)(c2ccccc2)c2ccccc2-4)cc1 ; N#Cc1cc(-n2c3ccccc3c3c2ccc2c4ccccc4n(-c4ccccc4)c23)ccc1-c1ccc(-n2c3ccccc3c3ccccc32)c(C#N)c1C#N ; CC1(C)c2ccccc2-c2ccc3c4ccccc4n(-c4cc(C#N)c(-c5cc(C#N)c(-n6c7ccccc7c7ccccc76)cc5C#N)cc4C#N)c3c21 ; N#Cc1cc(-n2c3ccccc3c3ccc4c(c5ccccc5n4-c4ccccc4)c32)c(C#N)cc1-c1cc(C#N)c(-n2c3ccccc3c3ccccc32)c(C#N)c1]

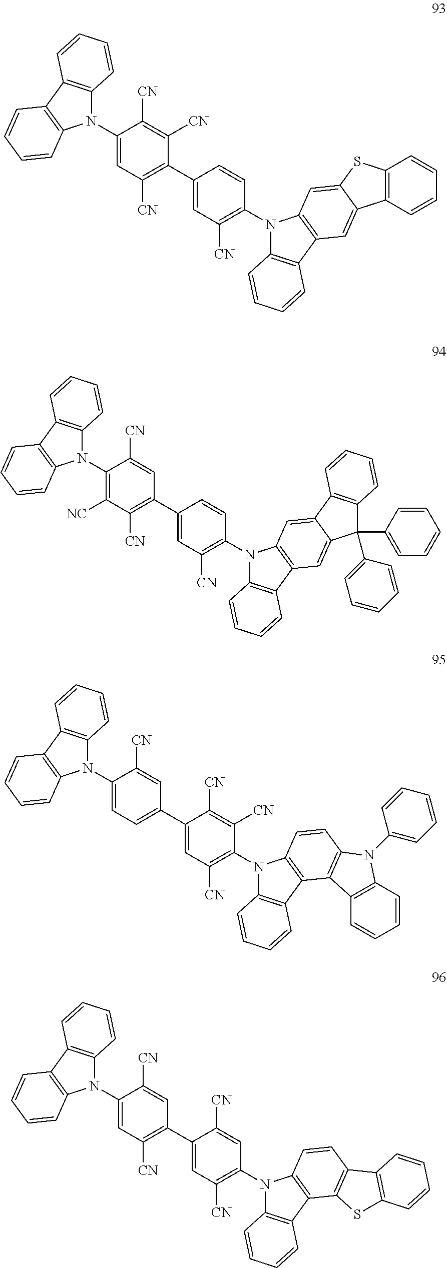 [SMILES: N#Cc1cc(-c2c(C#N)cc(-n3c4ccccc4c4ccccc43)c(C#N)c2C#N)ccc1-n1c2ccccc2c2cc3c(cc21)sc1ccccc13 ; N#Cc1cc(-c2cc(C#N)c(-n3c4ccccc4c4ccccc43)c(C#N)c2C#N)ccc1-n1c2ccccc2c2cc3c(cc21)-c1ccccc1C3(c1ccccc1)c1ccccc1 ; N#Cc1cc(-c2cc(C#N)c(-n3c4ccccc4c4c5c6ccccc6n(-c6ccccc6)c5ccc43)c(C#N)c2C#N)ccc1-n1c2ccccc2c2ccccc21 ; N#Cc1cc(-n2c3ccccc3c3ccccc32)c(C#N)cc1-c1cc(C#N)c(-n2c3ccccc3c3c4sc5ccccc5c4ccc32)cc1C#N]

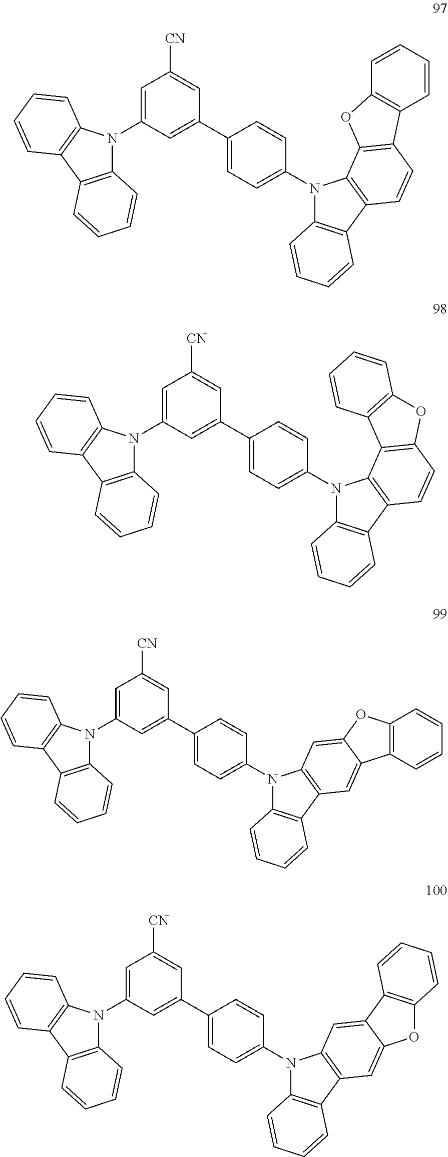 [SMILES: N#Cc1cc(-c2ccc(-n3c4ccccc4c4ccc5c6ccccc6oc5c43)cc2)cc(-n2c3ccccc3c3ccccc32)c1 ; N#Cc1cc(-c2ccc(-n3c4ccccc4c4ccc5oc6ccccc6c5c43)cc2)cc(-n2c3ccccc3c3ccccc32)c1 ; N#Cc1cc(-c2ccc(-n3c4ccccc4c4cc5c(cc43)oc3ccccc35)cc2)cc(-n2c3ccccc3c3ccccc32)c1 ; N#Cc1cc(-c2ccc(-n3c4ccccc4c4cc5oc6ccccc6c5cc43)cc2)cc(-n2c3ccccc3c3ccccc32)c1]

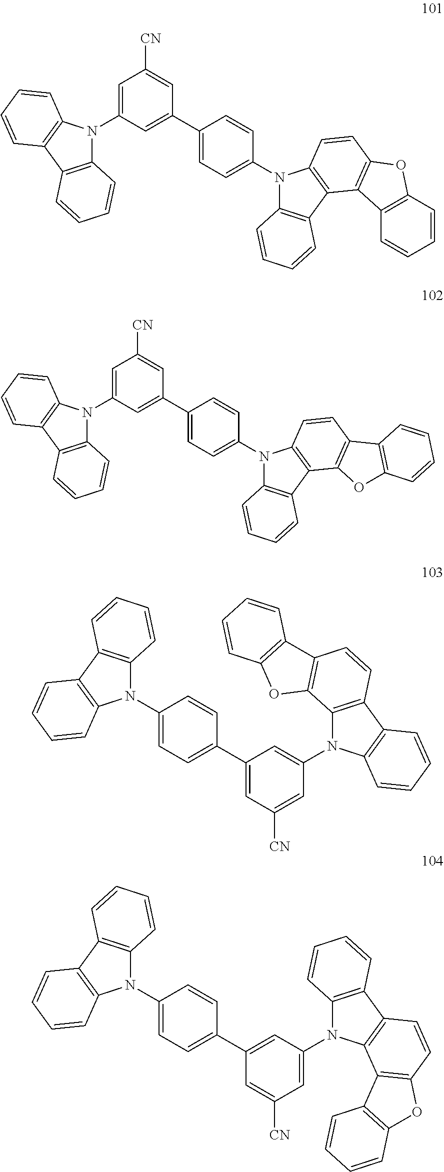 [SMILES: N#Cc1cc(-c2ccc(-n3c4ccccc4c4c5c(ccc43)oc3ccccc35)cc2)cc(-n2c3ccccc3c3ccccc32)c1 ; N#Cc1cc(-c2ccc(-n3c4ccccc4c4c5oc6ccccc6c5ccc43)cc2)cc(-n2c3ccccc3c3ccccc32)c1 ; N#Cc1cc(-c2ccc(-n3c4ccccc4c4ccccc43)cc2)cc(-n2c3ccccc3c3ccc4c5ccccc5oc4c32)c1 ; N#Cc1cc(-c2ccc(-n3c4ccccc4c4ccccc43)cc2)cc(-n2c3ccccc3c3ccc4oc5ccccc5c4c32)c1]

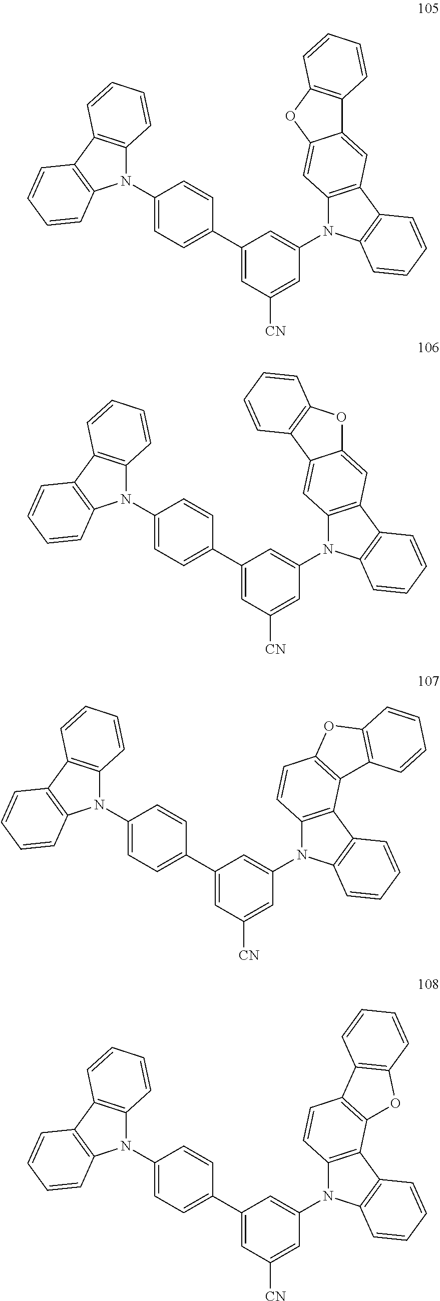

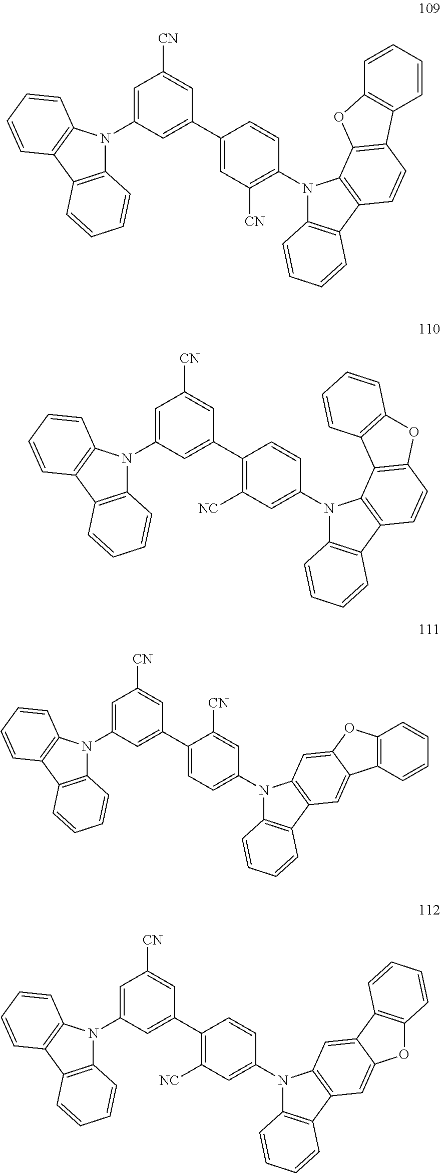 [SMILES: N#Cc1cc(-c2ccc(-n3c4ccccc4c4ccc5c6ccccc6oc5c43)c(C#N)c2)cc(-n2c3ccccc3c3ccccc32)c1 ; N#Cc1cc(-c2ccc(-n3c4ccccc4c4ccc5oc6ccccc6c5c43)cc2C#N)cc(-n2c3ccccc3c3ccccc32)c1 ; N#Cc1cc(-c2ccc(-n3c4ccccc4c4cc5c(cc43)oc3ccccc35)cc2C#N)cc(-n2c3ccccc3c3ccccc32)c1 ; N#Cc1cc(-c2ccc(-n3c4ccccc4c4cc5oc6ccccc6c5cc43)cc2C#N)cc(-n2c3ccccc3c3ccccc32)c1]

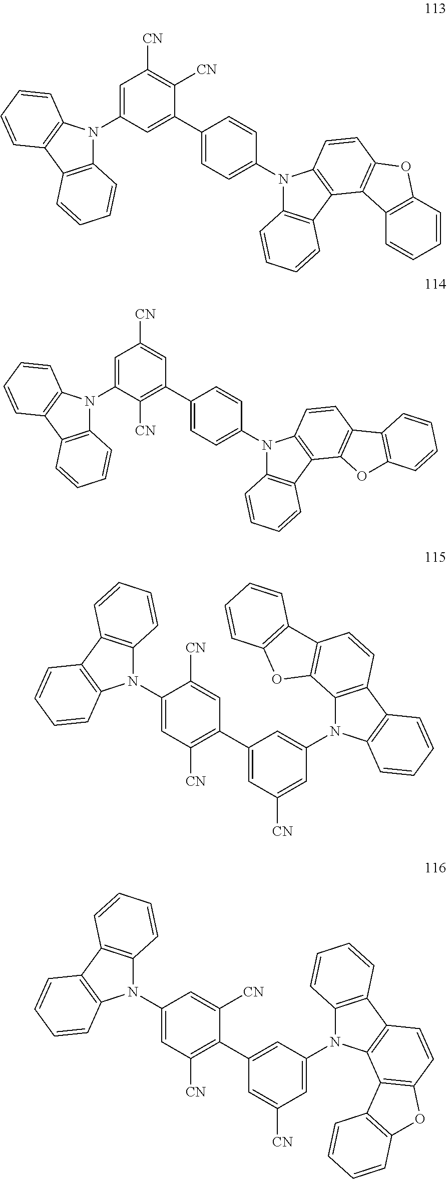 [SMILES: N#Cc1cc(-n2c3ccccc3c3ccccc32)cc(-c2ccc(-n3c4ccccc4c4c5c(ccc43)oc3ccccc35)cc2)c1C#N ; N#Cc1cc(-c2ccc(-n3c4ccccc4c4c5oc6ccccc6c5ccc43)cc2)c(C#N)c(-n2c3ccccc3c3ccccc32)c1 ; N#Cc1cc(-c2cc(C#N)c(-n3c4ccccc4c4ccccc43)cc2C#N)cc(-n2c3ccccc3c3ccc4c5ccccc5oc4c32)c1 ; N#Cc1cc(-c2c(C#N)cc(-n3c4ccccc4c4ccccc43)cc2C#N)cc(-n2c3ccccc3c3ccc4oc5ccccc5c4c32)c1]

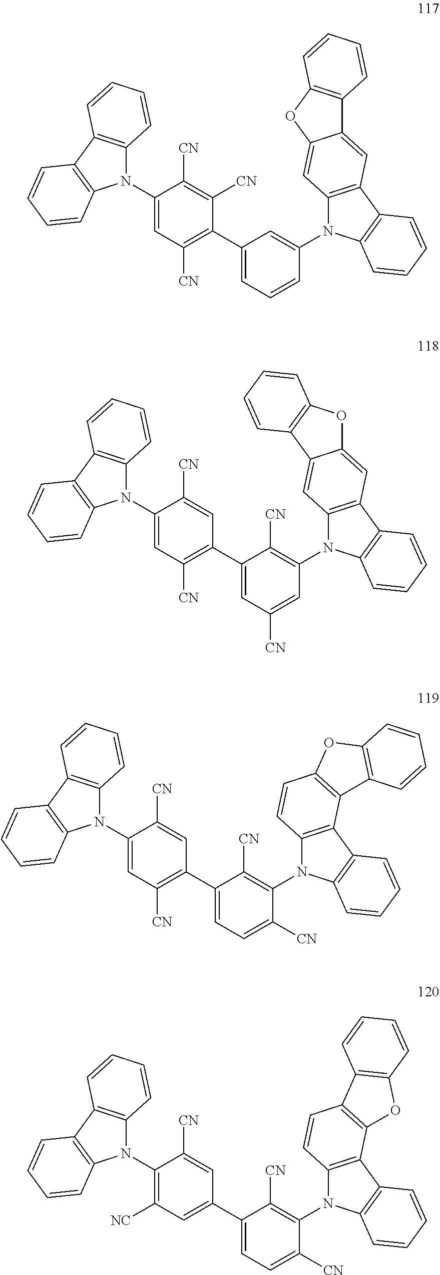 [SMILES: N#Cc1cc(-n2c3ccccc3c3ccccc32)c(C#N)c(C#N)c1-c1cccc(-n2c3ccccc3c3cc4c(cc32)oc2ccccc24)c1 ; N#Cc1cc(-c2cc(C#N)c(-n3c4ccccc4c4ccccc43)cc2C#N)c(C#N)c(-n2c3ccccc3c3cc4oc5ccccc5c4cc32)c1 ; N#Cc1cc(-n2c3ccccc3c3ccccc32)c(C#N)cc1-c1ccc(C#N)c(-n2c3ccccc3c3c4c(ccc32)oc2ccccc24)c1C#N ; N#Cc1cc(-c2ccc(C#N)c(-n3c4ccccc4c4c5oc6ccccc6c5ccc43)c2C#N)cc(C#N)c1-n1c2ccccc2c2ccccc21]

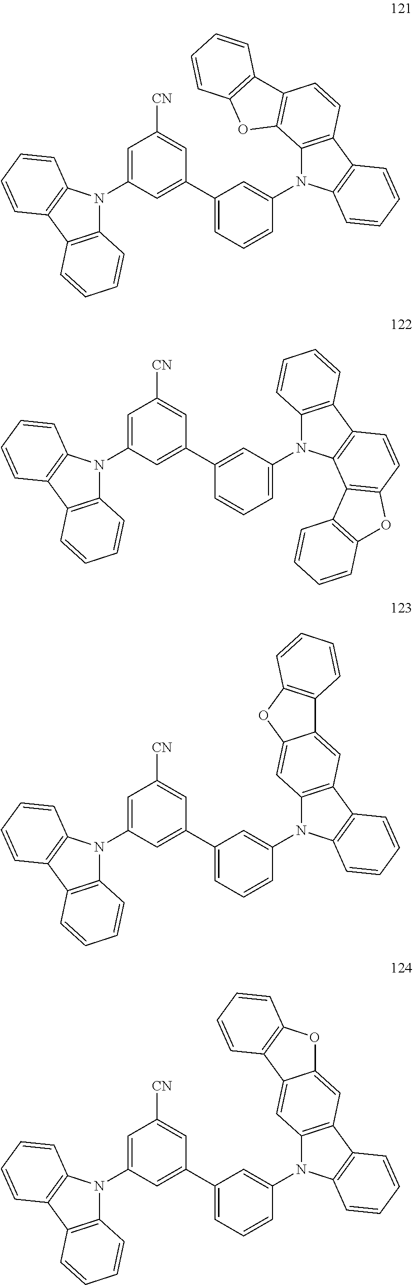 [SMILES: N#Cc1cc(-c2cccc(-n3c4ccccc4c4ccc5c6ccccc6oc5c43)c2)cc(-n2c3ccccc3c3ccccc32)c1 ; N#Cc1cc(-c2cccc(-n3c4ccccc4c4ccc5oc6ccccc6c5c43)c2)cc(-n2c3ccccc3c3ccccc32)c1 ; N#Cc1cc(-c2cccc(-n3c4ccccc4c4cc5c(cc43)oc3ccccc35)c2)cc(-n2c3ccccc3c3ccccc32)c1 ; N#Cc1cc(-c2cccc(-n3c4ccccc4c4cc5oc6ccccc6c5cc43)c2)cc(-n2c3ccccc3c3ccccc32)c1]

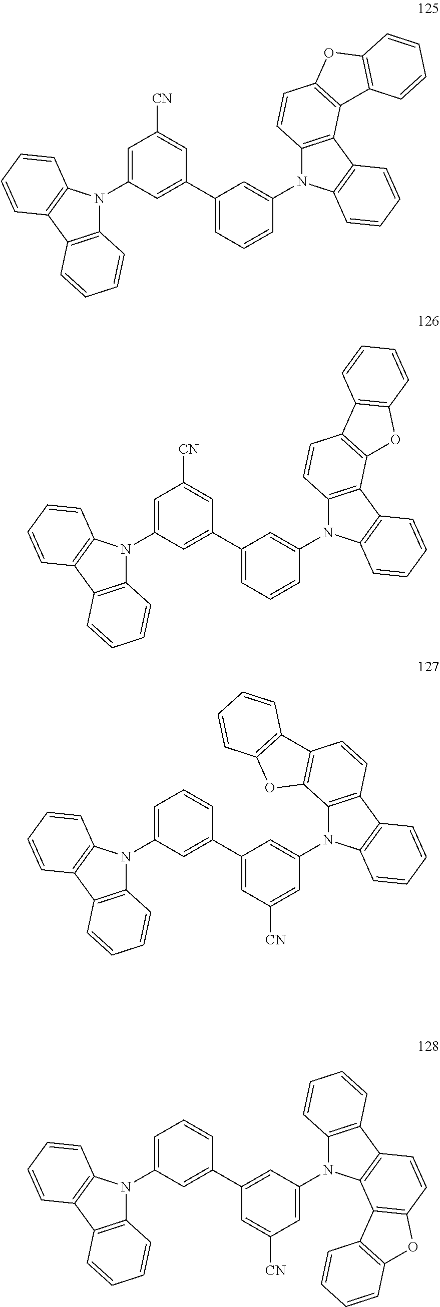 [SMILES: N#Cc1cc(-c2cccc(-n3c4ccccc4c4c5c(ccc43)oc3ccccc35)c2)cc(-n2c3ccccc3c3ccccc32)c1 ; N#Cc1cc(-c2cccc(-n3c4ccccc4c4c5oc6ccccc6c5ccc43)c2)cc(-n2c3ccccc3c3ccccc32)c1 ; N#Cc1cc(-c2cccc(-n3c4ccccc4c4ccccc43)c2)cc(-n2c3ccccc3c3ccc4c5ccccc5oc4c32)c1 ; N#Cc1cc(-c2cccc(-n3c4ccccc4c4ccccc43)c2)cc(-n2c3ccccc3c3ccc4oc5ccccc5c4c32)c1]

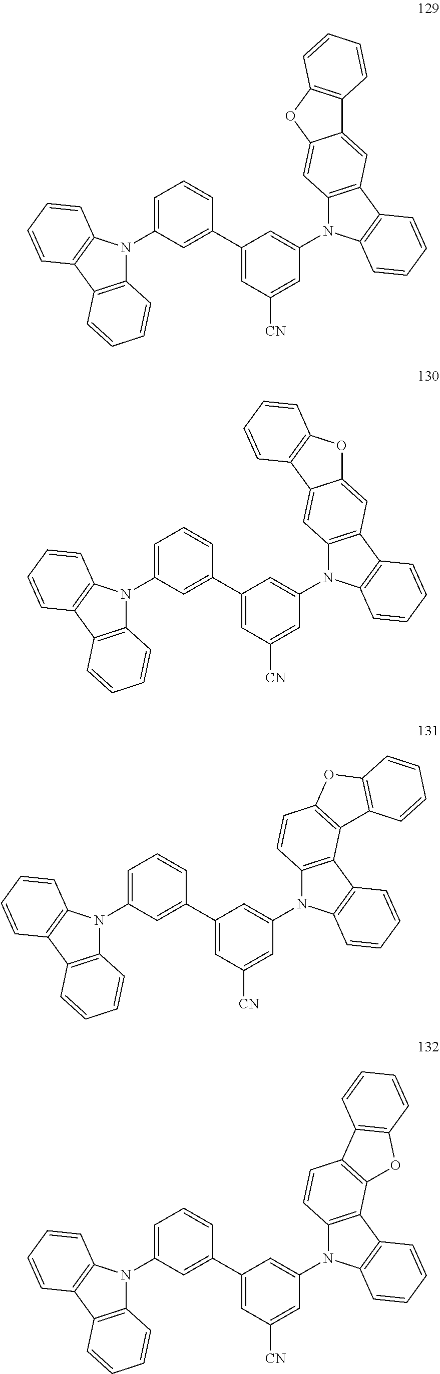 [SMILES: N#Cc1cc(-c2cccc(-n3c4ccccc4c4ccccc43)c2)cc(-n2c3ccccc3c3cc4c(cc32)oc2ccccc24)c1 ; N#Cc1cc(-c2cccc(-n3c4ccccc4c4ccccc43)c2)cc(-n2c3ccccc3c3cc4oc5ccccc5c4cc32)c1 ; N#Cc1cc(-c2cccc(-n3c4ccccc4c4ccccc43)c2)cc(-n2c3ccccc3c3c4c(ccc32)oc2ccccc24)c1 ; N#Cc1cc(-c2cccc(-n3c4ccccc4c4ccccc43)c2)cc(-n2c3ccccc3c3c4oc5ccccc5c4ccc32)c1]

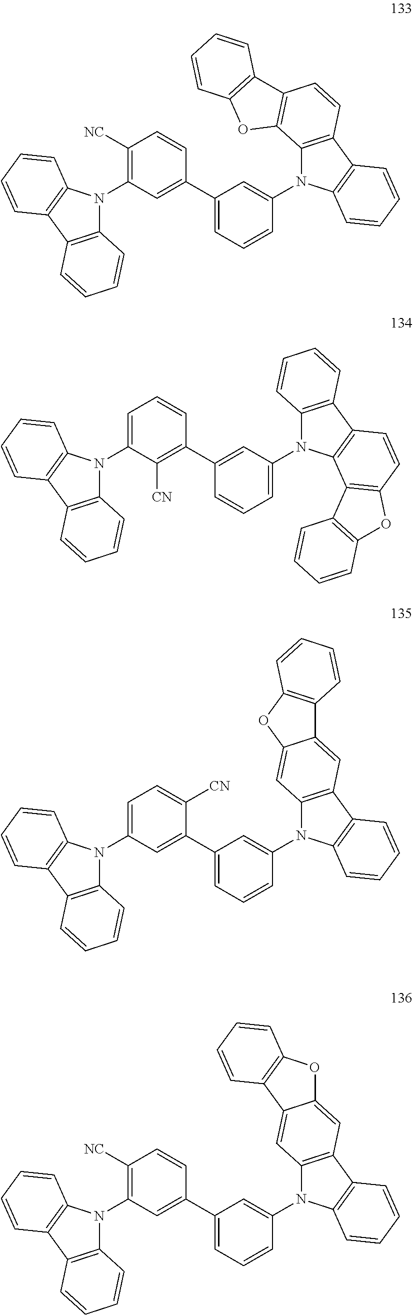 [SMILES: N#Cc1ccc(-c2cccc(-n3c4ccccc4c4ccc5c6ccccc6oc5c43)c2)cc1-n1c2ccccc2c2ccccc21 ; N#Cc1c(-c2cccc(-n3c4ccccc4c4ccc5oc6ccccc6c5c43)c2)cccc1-n1c2ccccc2c2ccccc21 ; N#Cc1ccc(-n2c3ccccc3c3ccccc32)cc1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)cc1-n1c2ccccc2c2ccccc21]

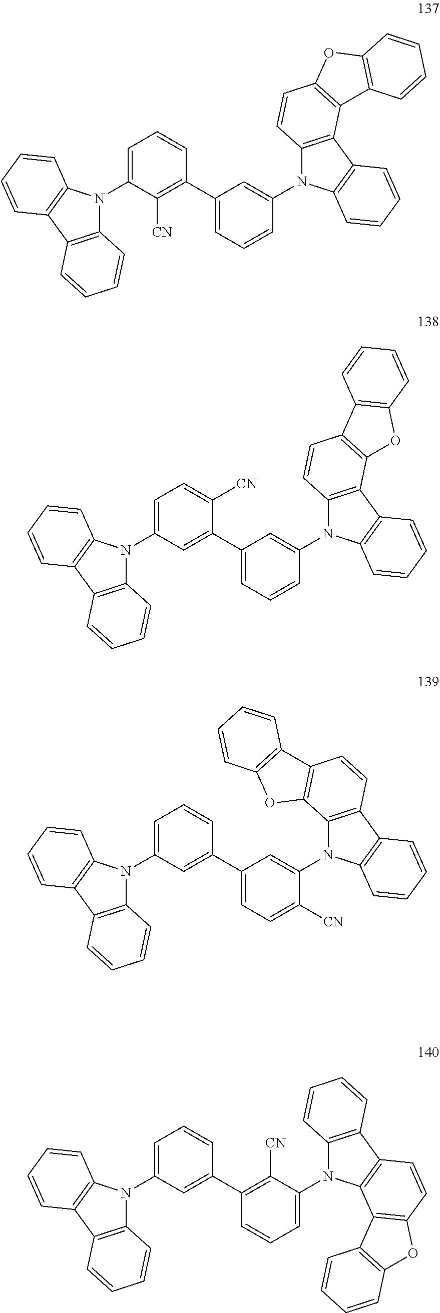 [SMILES: N#Cc1c(-c2cccc(-n3c4ccccc4c4c5c(ccc43)oc3ccccc35)c2)cccc1-n1c2ccccc2c2ccccc21 ; N#Cc1ccc(-n2c3ccccc3c3ccccc32)cc1-c1cccc(-n2c3ccccc3c3c4oc5ccccc5c4ccc32)c1 ; N#Cc1ccc(-c2cccc(-n3c4ccccc4c4ccccc43)c2)cc1-n1c2ccccc2c2ccc3c4ccccc4oc3c21 ; N#Cc1c(-c2cccc(-n3c4ccccc4c4ccccc43)c2)cccc1-n1c2ccccc2c2ccc3oc4ccccc4c3c21]

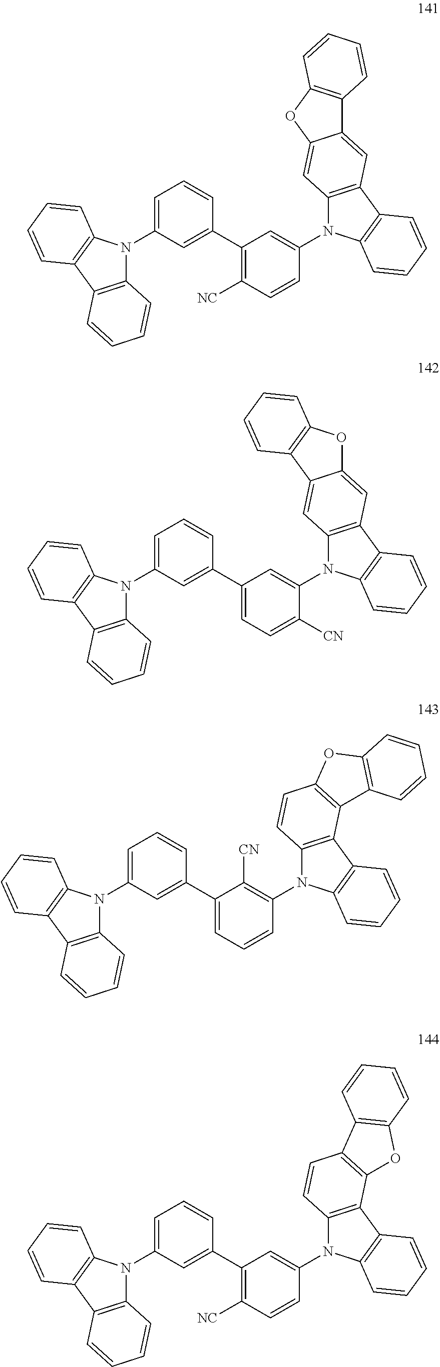 [SMILES: N#Cc1ccc(-n2c3ccccc3c3cc4c(cc32)oc2ccccc24)cc1-c1cccc(-n2c3ccccc3c3ccccc32)c1 ; N#Cc1ccc(-c2cccc(-n3c4ccccc4c4ccccc43)c2)cc1-n1c2ccccc2c2cc3oc4ccccc4c3cc21 ; N#Cc1c(-c2cccc(-n3c4ccccc4c4ccccc43)c2)cccc1-n1c2ccccc2c2c3c(ccc21)oc1ccccc13 ; N#Cc1ccc(-n2c3ccccc3c3c4oc5ccccc5c4ccc32)cc1-c1cccc(-n2c3ccccc3c3ccccc32)c1]

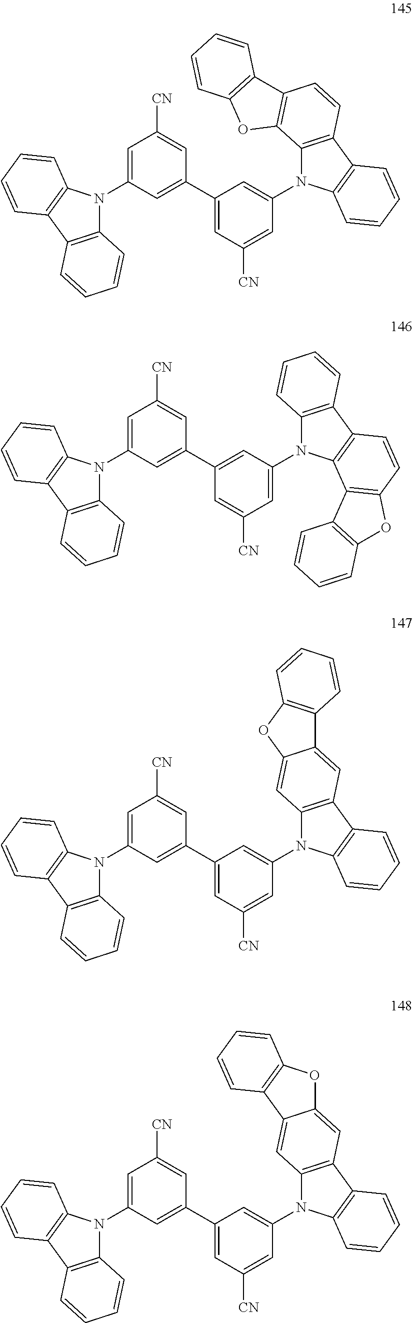 [SMILES: N#Cc1cc(-c2cc(C#N)cc(-n3c4ccccc4c4ccc5c6ccccc6oc5c43)c2)cc(-n2c3ccccc3c3ccccc32)c1 ; N#Cc1cc(-c2cc(C#N)cc(-n3c4ccccc4c4ccc5oc6ccccc6c5c43)c2)cc(-n2c3ccccc3c3ccccc32)c1 ; N#Cc1cc(-c2cc(C#N)cc(-n3c4ccccc4c4cc5c(cc43)oc3ccccc35)c2)cc(-n2c3ccccc3c3ccccc32)c1 ; N#Cc1cc(-c2cc(C#N)cc(-n3c4ccccc4c4cc5oc6ccccc6c5cc43)c2)cc(-n2c3ccccc3c3ccccc32)c1]

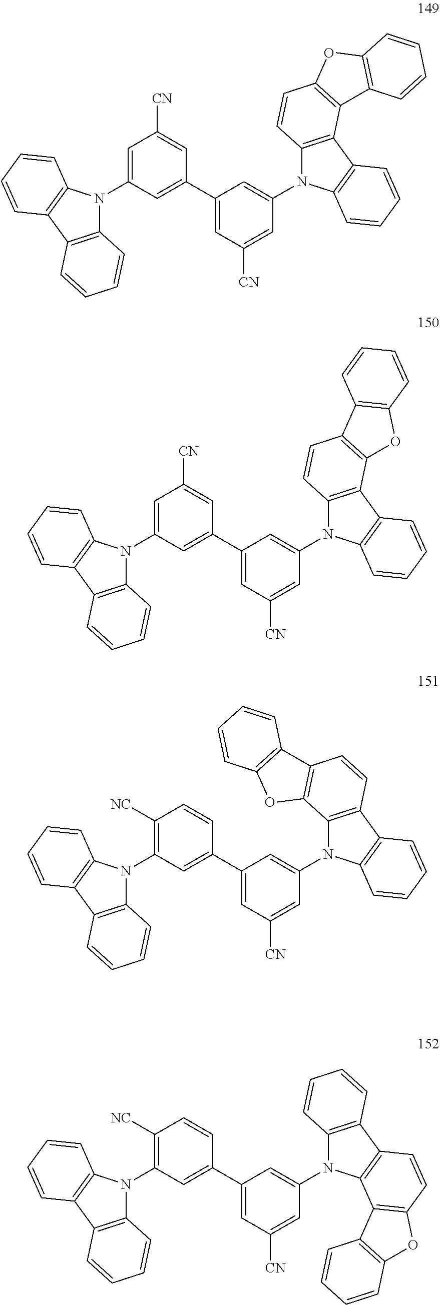 [SMILES: N#Cc1cc(-c2cc(C#N)cc(-n3c4ccccc4c4c5c(ccc43)oc3ccccc35)c2)cc(-n2c3ccccc3c3ccccc32)c1 ; N#Cc1cc(-c2cc(C#N)cc(-n3c4ccccc4c4c5oc6ccccc6c5ccc43)c2)cc(-n2c3ccccc3c3ccccc32)c1 ; N#Cc1cc(-c2ccc(C#N)c(-n3c4ccccc4c4ccccc43)c2)cc(-n2c3ccccc3c3ccc4c5ccccc5oc4c32)c1 ; N#Cc1cc(-c2ccc(C#N)c(-n3c4ccccc4c4ccccc43)c2)cc(-n2c3ccccc3c3ccc4oc5ccccc5c4c32)c1]

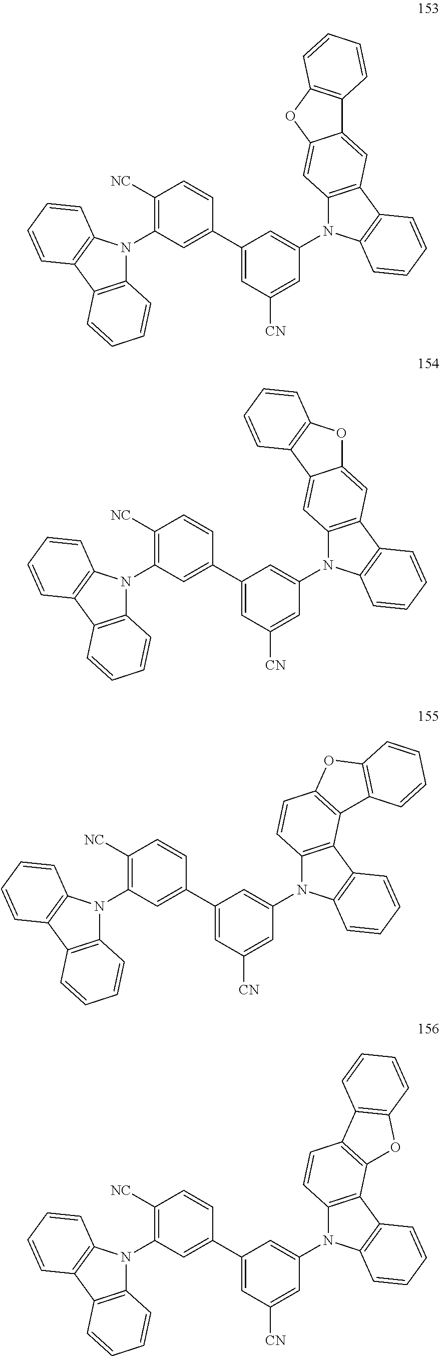 [SMILES: N#Cc1cc(-c2ccc(C#N)c(-n3c4ccccc4c4ccccc43)c2)cc(-n2c3ccccc3c3cc4c(cc32)oc2ccccc24)c1 ; N#Cc1cc(-c2ccc(C#N)c(-n3c4ccccc4c4ccccc43)c2)cc(-n2c3ccccc3c3cc4oc5ccccc5c4cc32)c1 ; N#Cc1cc(-c2ccc(C#N)c(-n3c4ccccc4c4ccccc43)c2)cc(-n2c3ccccc3c3c4c(ccc32)oc2ccccc24)c1 ; N#Cc1cc(-c2ccc(C#N)c(-n3c4ccccc4c4ccccc43)c2)cc(-n2c3ccccc3c3c4oc5ccccc5c4ccc32)c1]

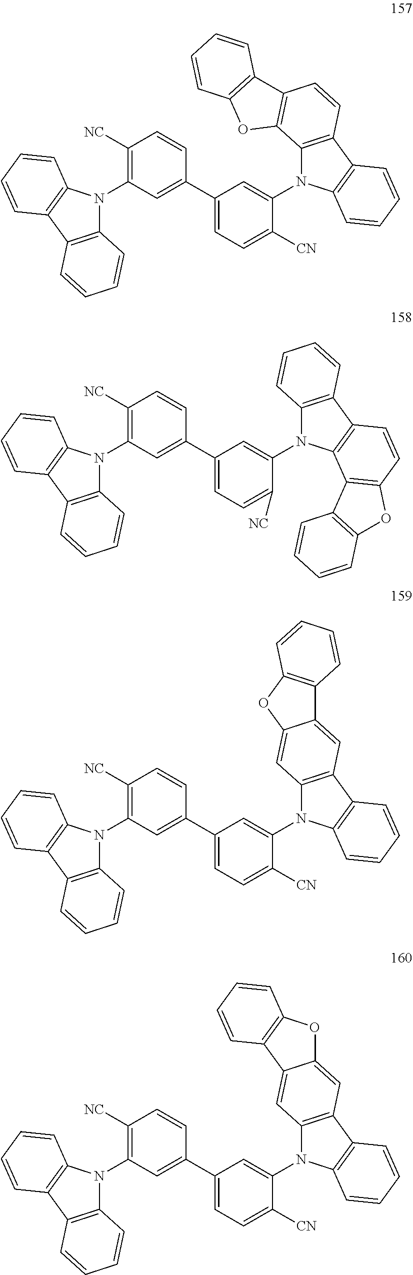 [SMILES: N#Cc1ccc(-c2ccc(C#N)c(-n3c4ccccc4c4ccc5c6ccccc6oc5c43)c2)cc1-n1c2ccccc2c2ccccc21 ; N#Cc1ccc(-c2ccc(C#N)c(-n3c4ccccc4c4ccc5oc6ccccc6c5c43)c2)cc1-n1c2ccccc2c2ccccc21 ; N#Cc1ccc(-c2ccc(C#N)c(-n3c4ccccc4c4cc5c(cc43)oc3ccccc35)c2)cc1-n1c2ccccc2c2ccccc21 ; N#Cc1ccc(-c2ccc(C#N)c(-n3c4ccccc4c4cc5oc6ccccc6c5cc43)c2)cc1-n1c2ccccc2c2ccccc21]

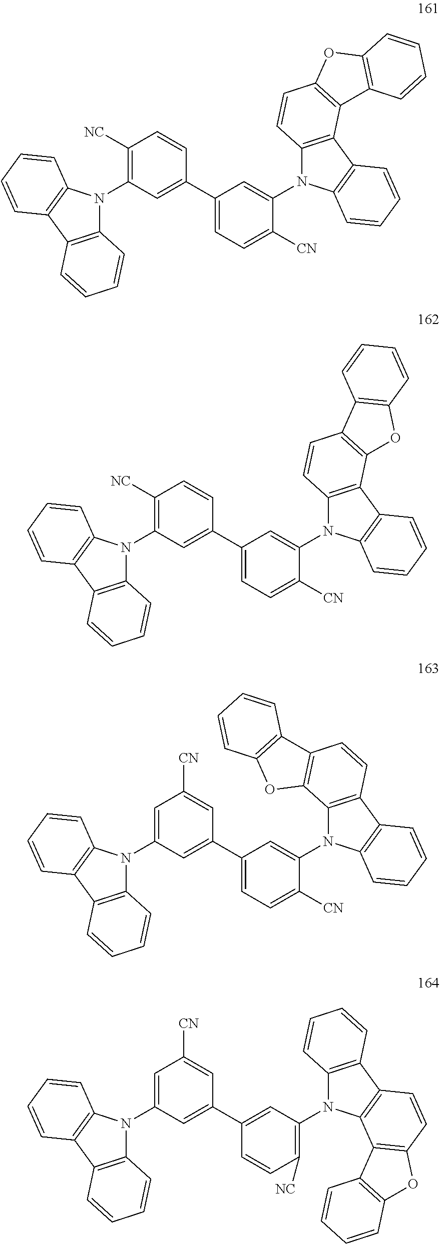 [SMILES: N#Cc1ccc(-c2ccc(C#N)c(-n3c4ccccc4c4c5c(ccc43)oc3ccccc35)c2)cc1-n1c2ccccc2c2ccccc21 ; N#Cc1ccc(-c2ccc(C#N)c(-n3c4ccccc4c4c5oc6ccccc6c5ccc43)c2)cc1-n1c2ccccc2c2ccccc21 ; N#Cc1cc(-c2ccc(C#N)c(-n3c4ccccc4c4ccc5c6ccccc6oc5c43)c2)cc(-n2c3ccccc3c3ccccc32)c1 ; N#Cc1cc(-c2ccc(C#N)c(-n3c4ccccc4c4ccc5oc6ccccc6c5c43)c2)cc(-n2c3ccccc3c3ccccc32)c1]

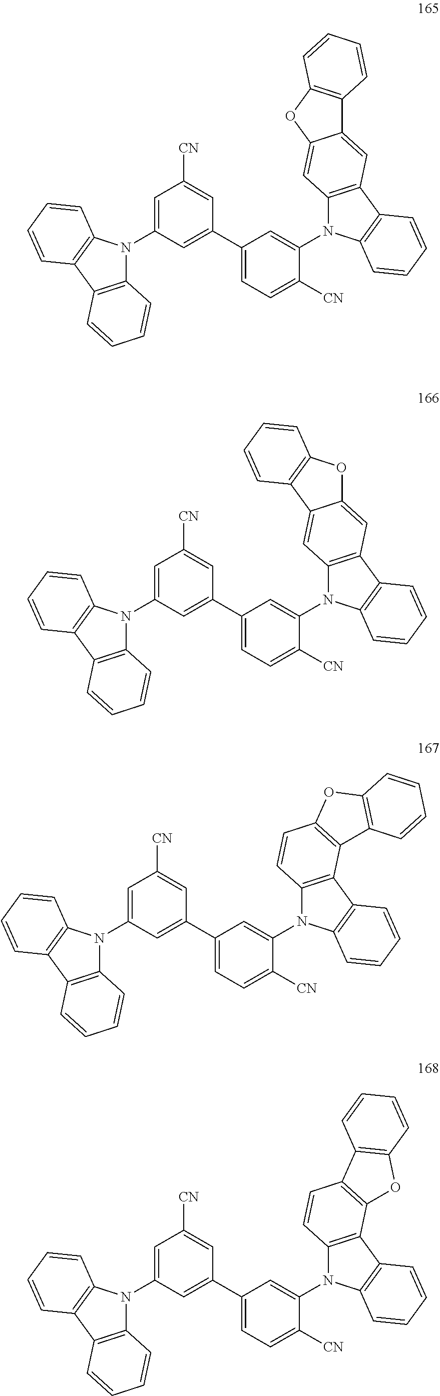 [SMILES: N#Cc1cc(-c2ccc(C#N)c(-n3c4ccccc4c4cc5c(cc43)oc3ccccc35)c2)cc(-n2c3ccccc3c3ccccc32)c1 ; N#Cc1cc(-c2ccc(C#N)c(-n3c4ccccc4c4cc5oc6ccccc6c5cc43)c2)cc(-n2c3ccccc3c3ccccc32)c1 ; N#Cc1cc(-c2ccc(C#N)c(-n3c4ccccc4c4c5c(ccc43)oc3ccccc35)c2)cc(-n2c3ccccc3c3ccccc32)c1 ; N#Cc1cc(-c2ccc(C#N)c(-n3c4ccccc4c4c5oc6ccccc6c5ccc43)c2)cc(-n2c3ccccc3c3ccccc32)c1]

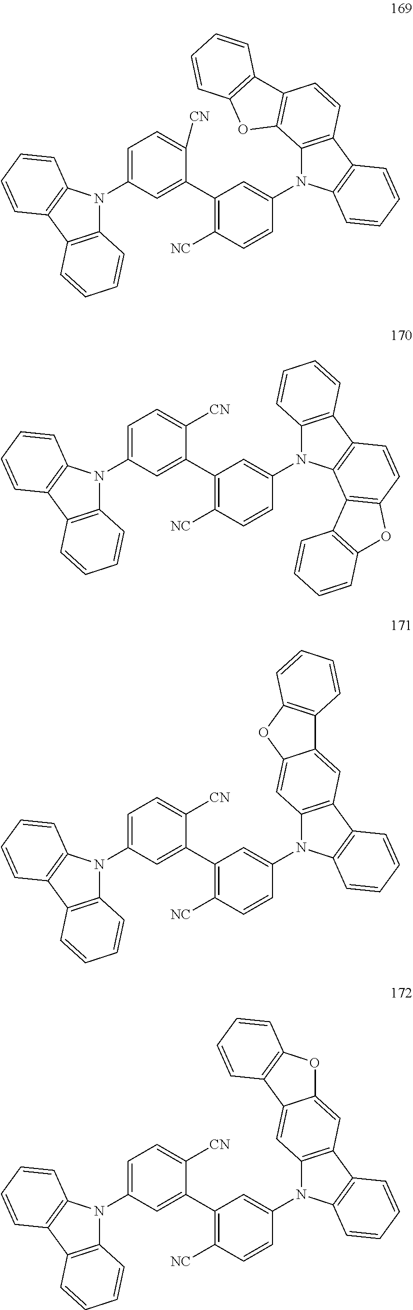 [SMILES: N#Cc1ccc(-n2c3ccccc3c3ccccc32)cc1-c1cc(-n2c3ccccc3c3ccc4c5ccccc5oc4c32)ccc1C#N ; N#Cc1ccc(-n2c3ccccc3c3ccccc32)cc1-c1cc(-n2c3ccccc3c3ccc4oc5ccccc5c4c32)ccc1C#N ; N#Cc1ccc(-n2c3ccccc3c3ccccc32)cc1-c1cc(-n2c3ccccc3c3cc4c(cc32)oc2ccccc24)ccc1C#N ; N#Cc1ccc(-n2c3ccccc3c3ccccc32)cc1-c1cc(-n2c3ccccc3c3cc4oc5ccccc5c4cc32)ccc1C#N]

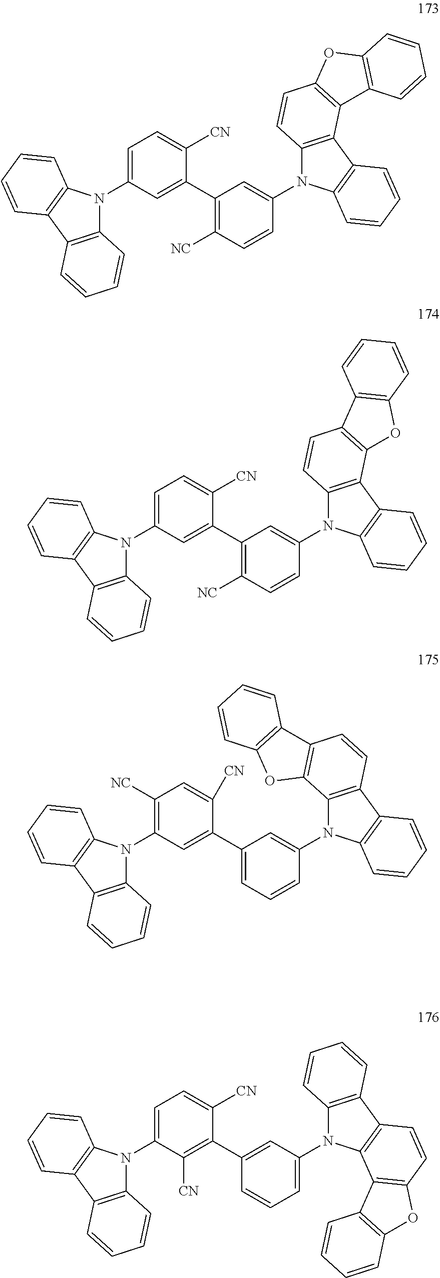 [SMILES: N#Cc1ccc(-n2c3ccccc3c3ccccc32)cc1-c1cc(-n2c3ccccc3c3c4c(ccc32)oc2ccccc24)ccc1C#N ; N#Cc1ccc(-n2c3ccccc3c3ccccc32)cc1-c1cc(-n2c3ccccc3c3c4oc5ccccc5c4ccc32)ccc1C#N ; N#Cc1cc(C#N)c(-n2c3ccccc3c3ccccc32)cc1-c1cccc(-n2c3ccccc3c3ccc4c5ccccc5oc4c32)c1 ; N#Cc1ccc(-n2c3ccccc3c3ccccc32)c(C#N)c1-c1cccc(-n2c3ccccc3c3ccc4oc5ccccc5c4c32)c1]

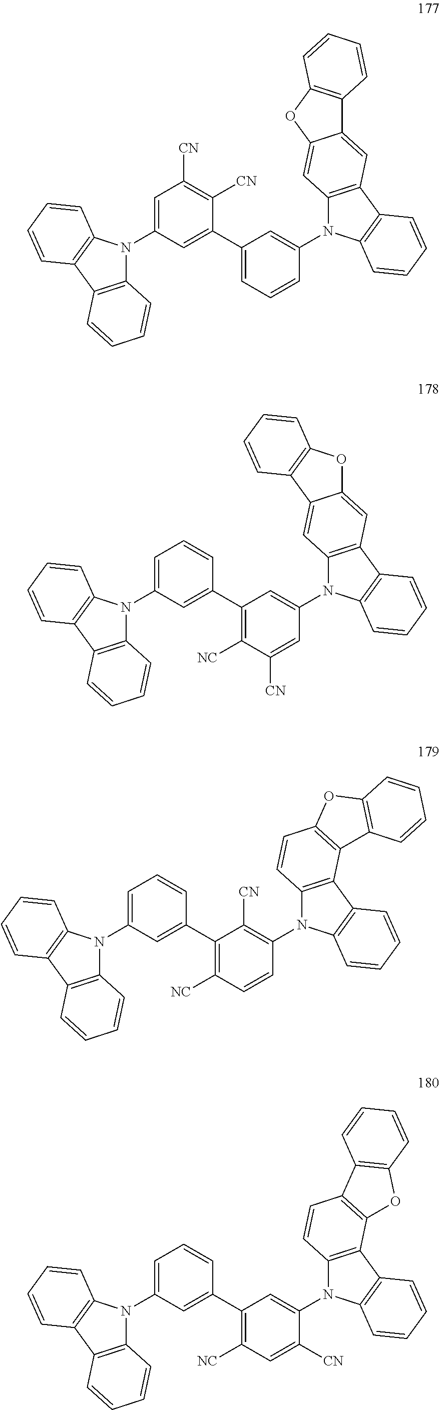 [SMILES: N#Cc1cc(-n2c3ccccc3c3ccccc32)cc(-c2cccc(-n3c4ccccc4c4cc5c(cc43)oc3ccccc35)c2)c1C#N ; N#Cc1cc(-n2c3ccccc3c3cc4oc5ccccc5c4cc32)cc(-c2cccc(-n3c4ccccc4c4ccccc43)c2)c1C#N ; N#Cc1ccc(-n2c3ccccc3c3c4c(ccc32)oc2ccccc24)c(C#N)c1-c1cccc(-n2c3ccccc3c3ccccc32)c1 ; N#Cc1cc(C#N)c(-n2c3ccccc3c3c4oc5ccccc5c4ccc32)cc1-c1cccc(-n2c3ccccc3c3ccccc32)c1]

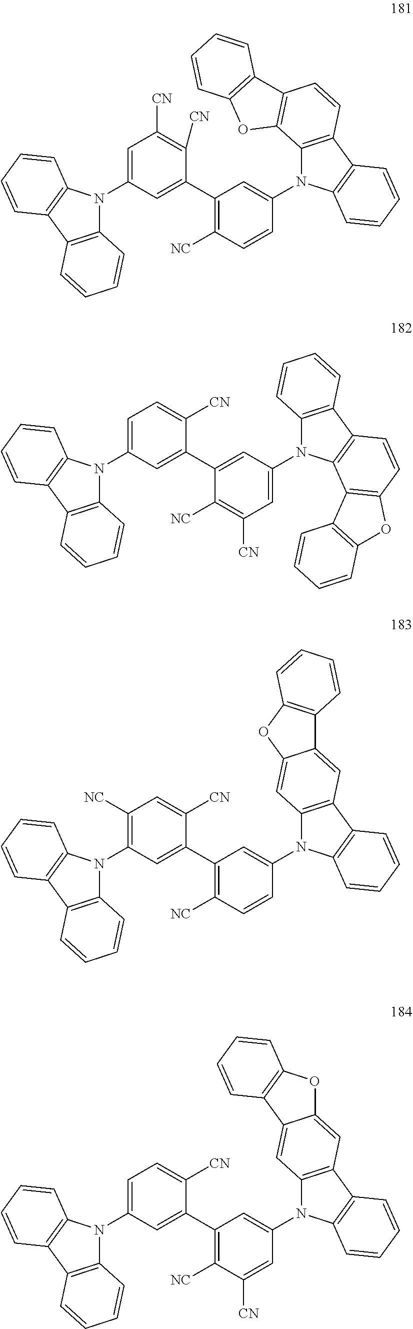 [SMILES: N#Cc1ccc(-n2c3ccccc3c3ccc4c5ccccc5oc4c32)cc1-c1cc(-n2c3ccccc3c3ccccc32)cc(C#N)c1C#N ; N#Cc1ccc(-n2c3ccccc3c3ccccc32)cc1-c1cc(-n2c3ccccc3c3ccc4oc5ccccc5c4c32)cc(C#N)c1C#N ; N#Cc1ccc(-n2c3ccccc3c3cc4c(cc32)oc2ccccc24)cc1-c1cc(-n2c3ccccc3c3ccccc32)c(C#N)cc1C#N ; N#Cc1ccc(-n2c3ccccc3c3ccccc32)cc1-c1cc(-n2c3ccccc3c3cc4oc5ccccc5c4cc32)cc(C#N)c1C#N]

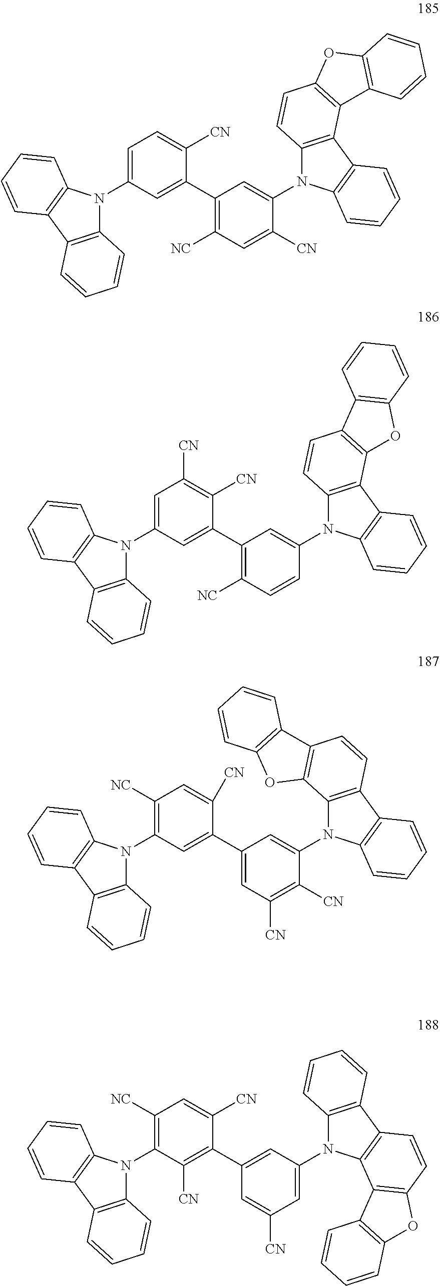 [SMILES: N#Cc1ccc(-n2c3ccccc3c3ccccc32)cc1-c1cc(-n2c3ccccc3c3c4c(ccc32)oc2ccccc24)c(C#N)cc1C#N ; N#Cc1ccc(-n2c3ccccc3c3c4oc5ccccc5c4ccc32)cc1-c1cc(-n2c3ccccc3c3ccccc32)cc(C#N)c1C#N ; N#Cc1cc(C#N)c(-n2c3ccccc3c3ccccc32)cc1-c1cc(C#N)c(C#N)c(-n2c3ccccc3c3ccc4c5ccccc5oc4c32)c1 ; N#Cc1cc(-c2c(C#N)cc(C#N)c(-n3c4ccccc4c4ccccc43)c2C#N)cc(-n2c3ccccc3c3ccc4oc5ccccc5c4c32)c1]

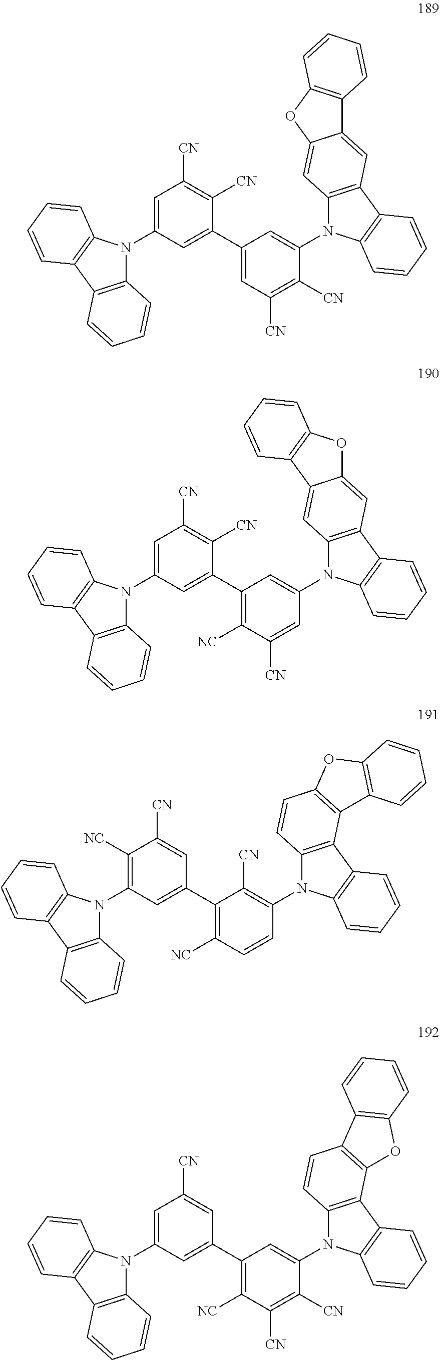 [SMILES: N#Cc1cc(-n2c3ccccc3c3ccccc32)cc(-c2cc(C#N)c(C#N)c(-n3c4ccccc4c4cc5c(cc43)oc3ccccc35)c2)c1C#N ; N#Cc1cc(-n2c3ccccc3c3ccccc32)cc(-c2cc(-n3c4ccccc4c4cc5oc6ccccc6c5cc43)cc(C#N)c2C#N)c1C#N ; N#Cc1cc(-c2c(C#N)ccc(-n3c4ccccc4c4c5c(ccc43)oc3ccccc35)c2C#N)cc(-n2c3ccccc3c3ccccc32)c1C#N ; N#Cc1cc(-c2cc(-n3c4ccccc4c4c5oc6ccccc6c5ccc43)c(C#N)c(C#N)c2C#N)cc(-n2c3ccccc3c3ccccc32)c1]

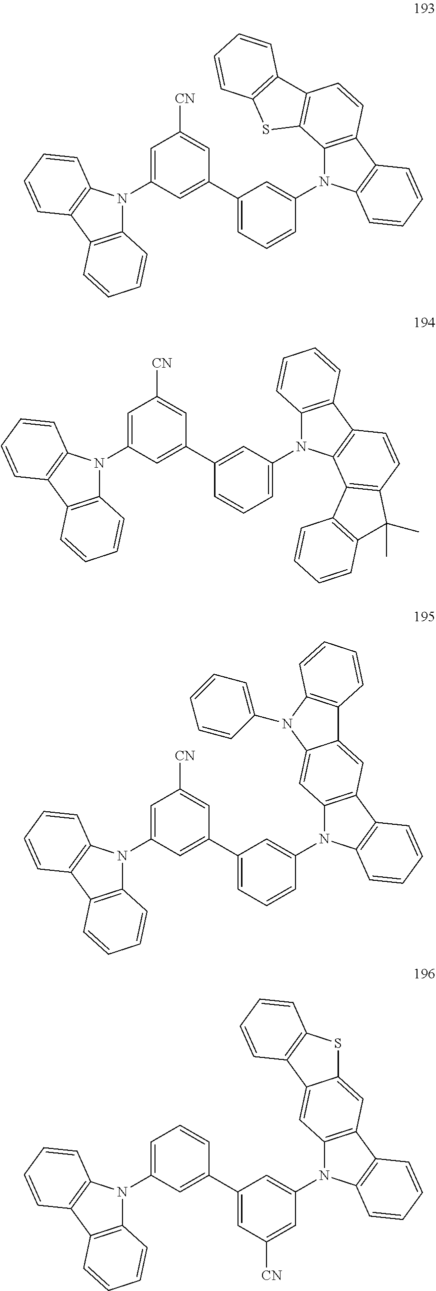 [SMILES: N#Cc1cc(-c2cccc(-n3c4ccccc4c4ccc5c6ccccc6sc5c43)c2)cc(-n2c3ccccc3c3ccccc32)c1 ; CC1(C)c2ccccc2-c2c1ccc1c3ccccc3n(-c3cccc(-c4cc(C#N)cc(-n5c6ccccc6c6ccccc65)c4)c3)c21 ; N#Cc1cc(-c2cccc(-n3c4ccccc4c4cc5c6ccccc6n(-c6ccccc6)c5cc43)c2)cc(-n2c3ccccc3c3ccccc32)c1 ; N#Cc1cc(-c2cccc(-n3c4ccccc4c4ccccc43)c2)cc(-n2c3ccccc3c3cc4sc5ccccc5c4cc32)c1]

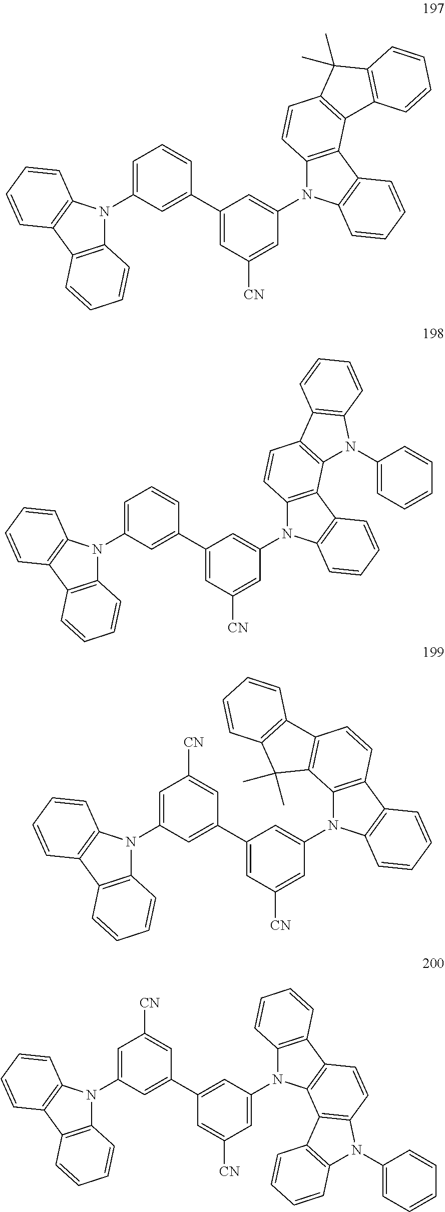 [SMILES: CC1(C)c2ccccc2-c2c1ccc1c2c2ccccc2n1-c1cc(C#N)cc(-c2cccc(-n3c4ccccc4c4ccccc43)c2)c1 ; N#Cc1cc(-c2cccc(-n3c4ccccc4c4ccccc43)c2)cc(-n2c3ccccc3c3c2ccc2c4ccccc4n(-c4ccccc4)c23)c1 ; CC1(C)c2ccccc2-c2ccc3c4ccccc4n(-c4cc(C#N)cc(-c5cc(C#N)cc(-n6c7ccccc7c7ccccc76)c5)c4)c3c21 ; N#Cc1cc(-c2cc(C#N)cc(-n3c4ccccc4c4ccc5c(c6ccccc6n5-c5ccccc5)c43)c2)cc(-n2c3ccccc3c3ccccc32)c1]

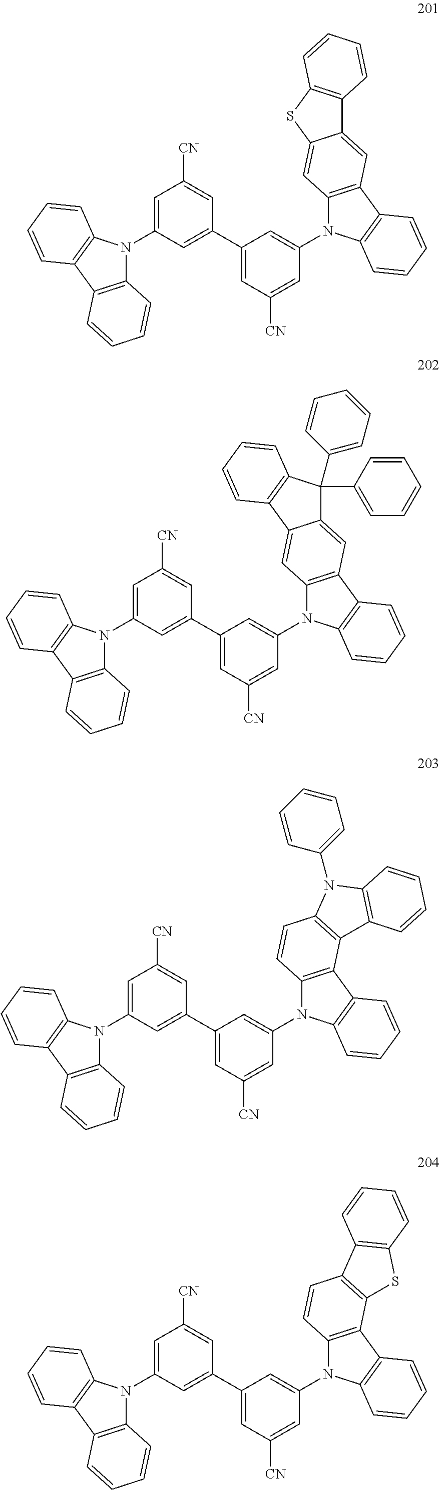 [SMILES: N#Cc1cc(-c2cc(C#N)cc(-n3c4ccccc4c4cc5c(cc43)sc3ccccc35)c2)cc(-n2c3ccccc3c3ccccc32)c1 ; N#Cc1cc(-c2cc(C#N)cc(-n3c4ccccc4c4cc5c(cc43)-c3ccccc3C5(c3ccccc3)c3ccccc3)c2)cc(-n2c3ccccc3c3ccccc32)c1 ; N#Cc1cc(-c2cc(C#N)cc(-n3c4ccccc4c4c5c6ccccc6n(-c6ccccc6)c5ccc43)c2)cc(-n2c3ccccc3c3ccccc32)c1 ; N#Cc1cc(-c2cc(C#N)cc(-n3c4ccccc4c4c5sc6ccccc6c5ccc43)c2)cc(-n2c3ccccc3c3ccccc32)c1]

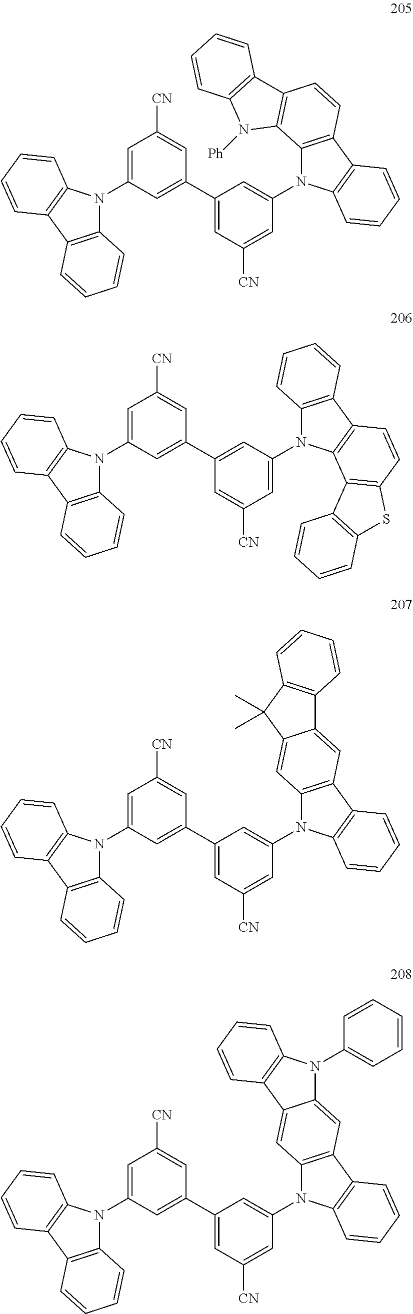 [SMILES: N#Cc1cc(-c2cc(C#N)cc(-n3c4ccccc4c4ccc5c6ccccc6n(-c6ccccc6)c5c43)c2)cc(-n2c3ccccc3c3ccccc32)c1 ; N#Cc1cc(-c2cc(C#N)cc(-n3c4ccccc4c4ccc5sc6ccccc6c5c43)c2)cc(-n2c3ccccc3c3ccccc32)c1 ; CC1(C)c2ccccc2-c2cc3c4ccccc4n(-c4cc(C#N)cc(-c5cc(C#N)cc(-n6c7ccccc7c7ccccc76)c5)c4)c3cc21 ; N#Cc1cc(-c2cc(C#N)cc(-n3c4ccccc4c4cc5c(cc43)c3ccccc3n5-c3ccccc3)c2)cc(-n2c3ccccc3c3ccccc32)c1]

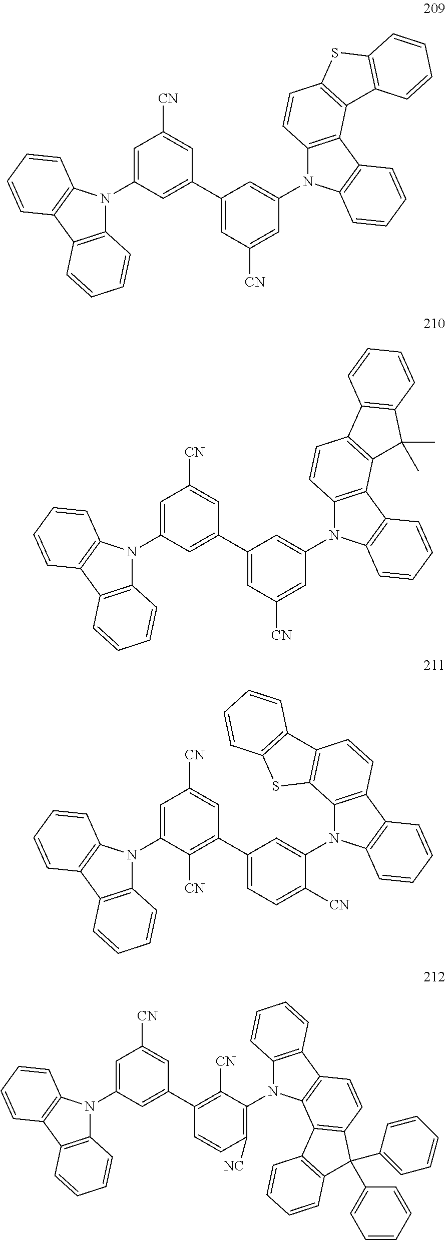 [SMILES: N#Cc1cc(-c2cc(C#N)cc(-n3c4ccccc4c4c5c(ccc43)sc3ccccc35)c2)cc(-n2c3ccccc3c3ccccc32)c1 ; CC1(C)c2ccccc2-c2ccc3c(c21)c1ccccc1n3-c1cc(C#N)cc(-c2cc(C#N)cc(-n3c4ccccc4c4ccccc43)c2)c1 ; N#Cc1cc(-c2ccc(C#N)c(-n3c4ccccc4c4ccc5c6ccccc6sc5c43)c2)c(C#N)c(-n2c3ccccc3c3ccccc32)c1 ; N#Cc1cc(-c2ccc(C#N)c(-n3c4ccccc4c4ccc5c(c43)-c3ccccc3C5(c3ccccc3)c3ccccc3)c2C#N)cc(-n2c3ccccc3c3ccccc32)c1]

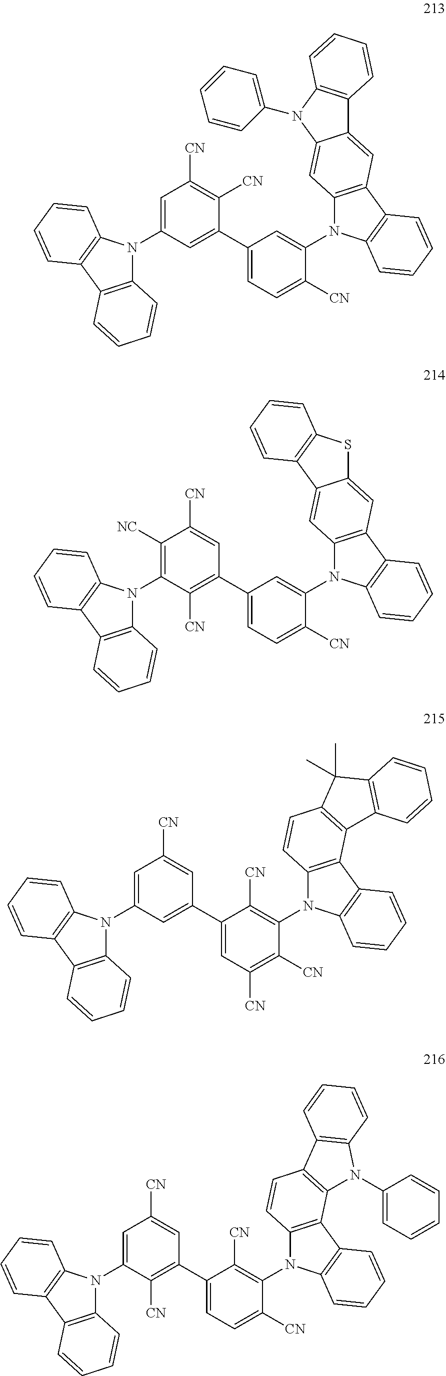 [SMILES: N#Cc1ccc(-c2cc(-n3c4ccccc4c4ccccc43)cc(C#N)c2C#N)cc1-n1c2ccccc2c2cc3c4ccccc4n(-c4ccccc4)c3cc21 ; N#Cc1ccc(-c2cc(C#N)c(C#N)c(-n3c4ccccc4c4ccccc43)c2C#N)cc1-n1c2ccccc2c2cc3sc4ccccc4c3cc21 ; CC1(C)c2ccccc2-c2c1ccc1c2c2ccccc2n1-c1c(C#N)c(C#N)cc(-c2cc(C#N)cc(-n3c4ccccc4c4ccccc43)c2)c1C#N ; N#Cc1cc(-c2ccc(C#N)c(-n3c4ccccc4c4c3ccc3c5ccccc5n(-c5ccccc5)c34)c2C#N)c(C#N)c(-n2c3ccccc3c3ccccc32)c1]

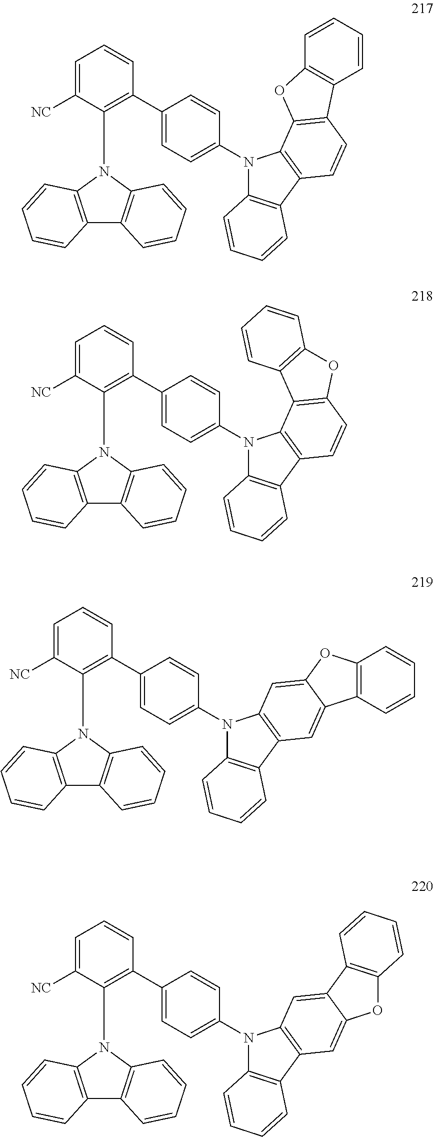

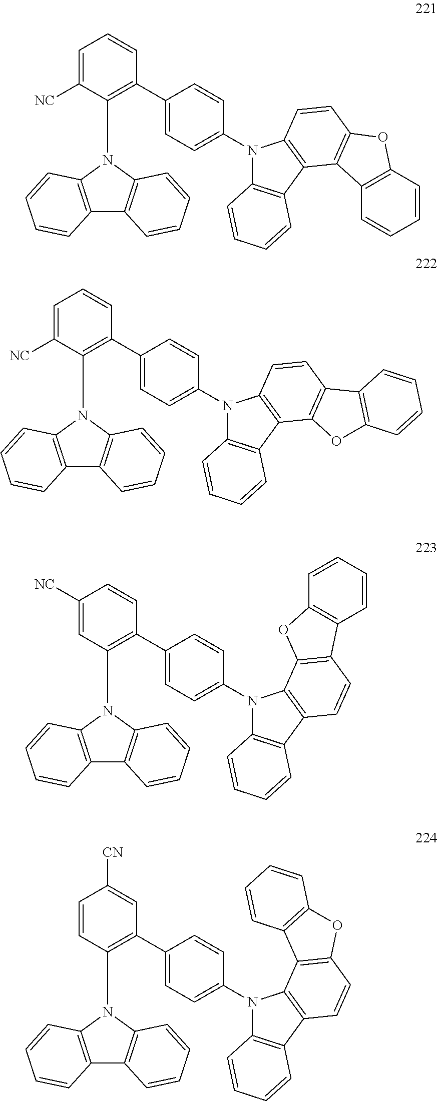 [SMILES: N#Cc1cccc(-c2ccc(-n3c4ccccc4c4c5c(ccc43)oc3ccccc35)cc2)c1-n1c2ccccc2c2ccccc21 ; N#Cc1cccc(-c2ccc(-n3c4ccccc4c4c5oc6ccccc6c5ccc43)cc2)c1-n1c2ccccc2c2ccccc21 ; N#Cc1ccc(-c2ccc(-n3c4ccccc4c4ccc5c6ccccc6oc5c43)cc2)c(-n2c3ccccc3c3ccccc32)c1 ; N#Cc1ccc(-n2c3ccccc3c3ccccc32)c(-c2ccc(-n3c4ccccc4c4ccc5oc6ccccc6c5c43)cc2)c1]

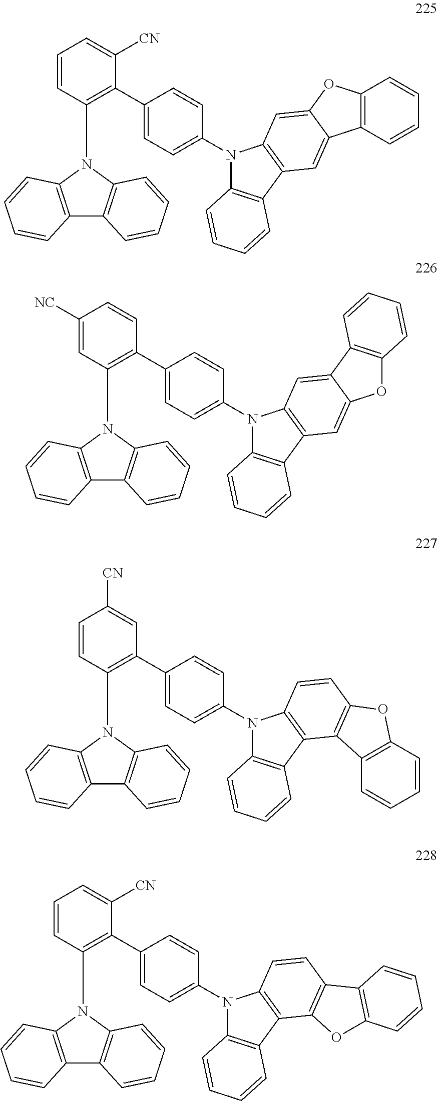 [SMILES: N#Cc1cccc(-n2c3ccccc3c3ccccc32)c1-c1ccc(-n2c3ccccc3c3cc4c(cc32)oc2ccccc24)cc1 ; N#Cc1ccc(-c2ccc(-n3c4ccccc4c4cc5oc6ccccc6c5cc43)cc2)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)cc2)c1 ; N#Cc1cccc(-n2c3ccccc3c3ccccc32)c1-c1ccc(-n2c3ccccc3c3c4oc5ccccc5c4ccc32)cc1]

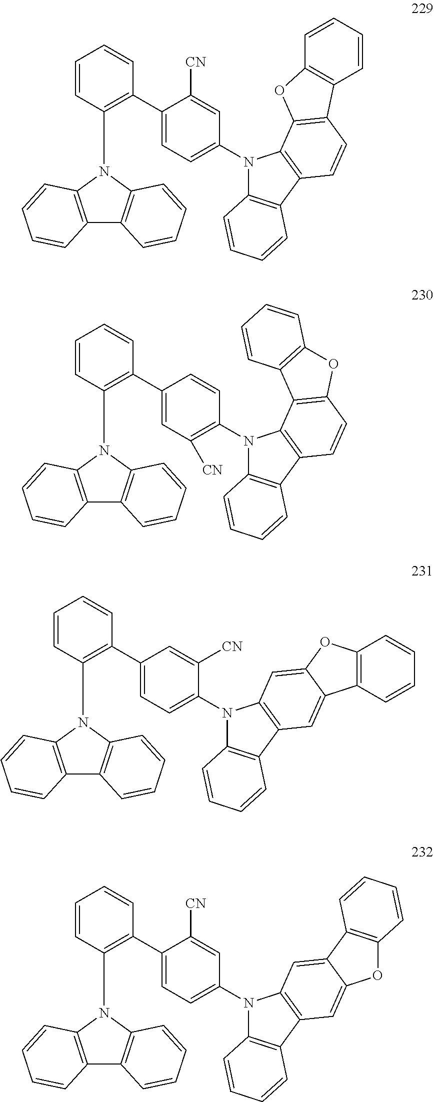

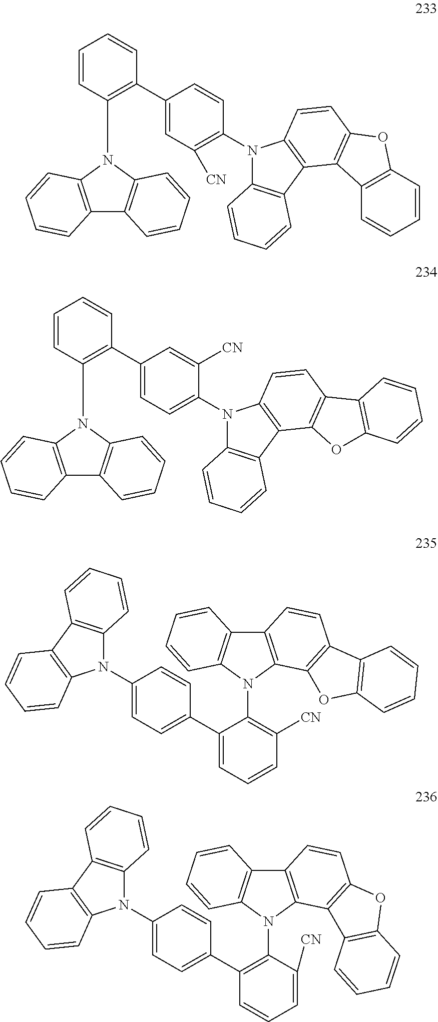 [SMILES: N#Cc1cc(-c2ccccc2-n2c3ccccc3c3ccccc32)ccc1-n1c2ccccc2c2c3c(ccc21)oc1ccccc13 ; N#Cc1cc(-c2ccccc2-n2c3ccccc3c3ccccc32)ccc1-n1c2ccccc2c2c3oc4ccccc4c3ccc21 ; N#Cc1cccc(-c2ccc(-n3c4ccccc4c4ccccc43)cc2)c1-n1c2ccccc2c2ccc3c4ccccc4oc3c21 ; N#Cc1cccc(-c2ccc(-n3c4ccccc4c4ccccc43)cc2)c1-n1c2ccccc2c2ccc3oc4ccccc4c3c21]

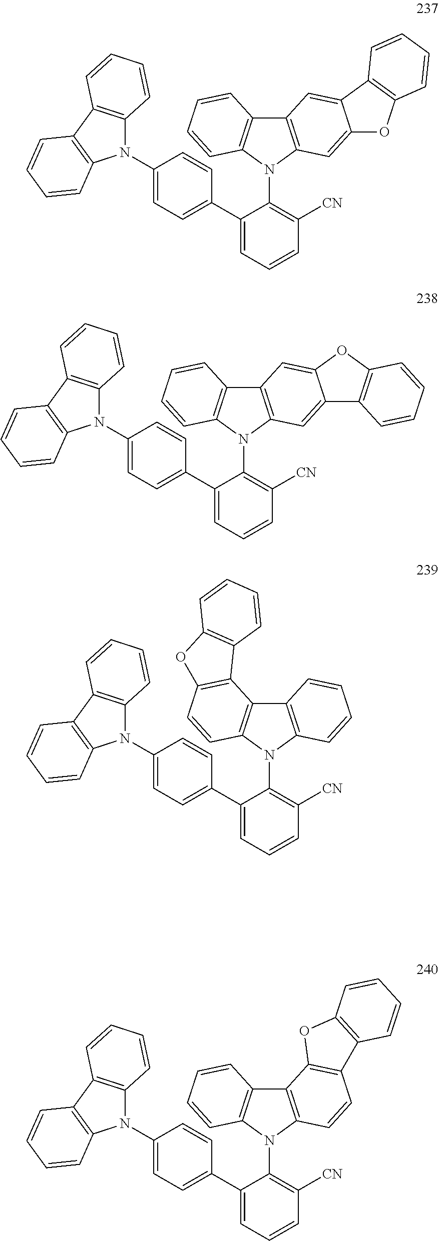

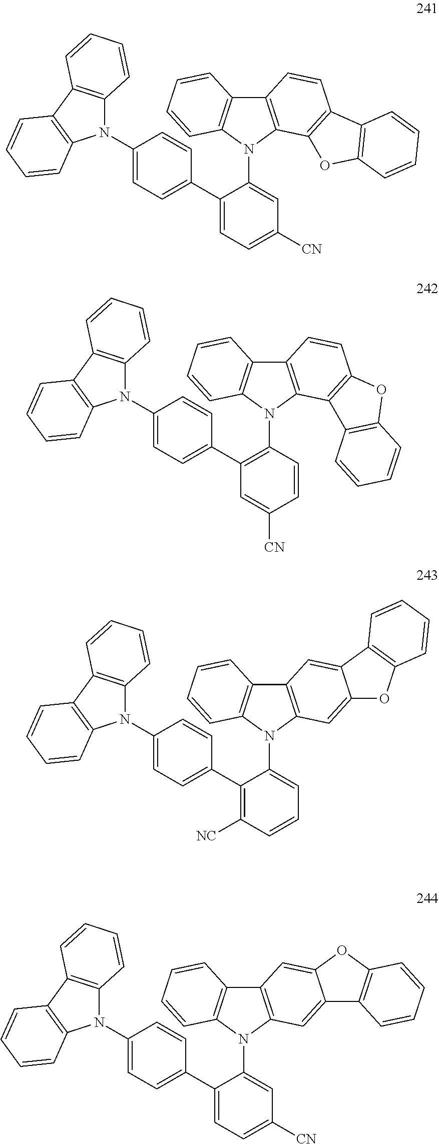 [SMILES: N#Cc1ccc(-c2ccc(-n3c4ccccc4c4ccccc43)cc2)c(-n2c3ccccc3c3ccc4c5ccccc5oc4c32)c1 ; N#Cc1ccc(-n2c3ccccc3c3ccc4oc5ccccc5c4c32)c(-c2ccc(-n3c4ccccc4c4ccccc43)cc2)c1 ; N#Cc1cccc(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1]

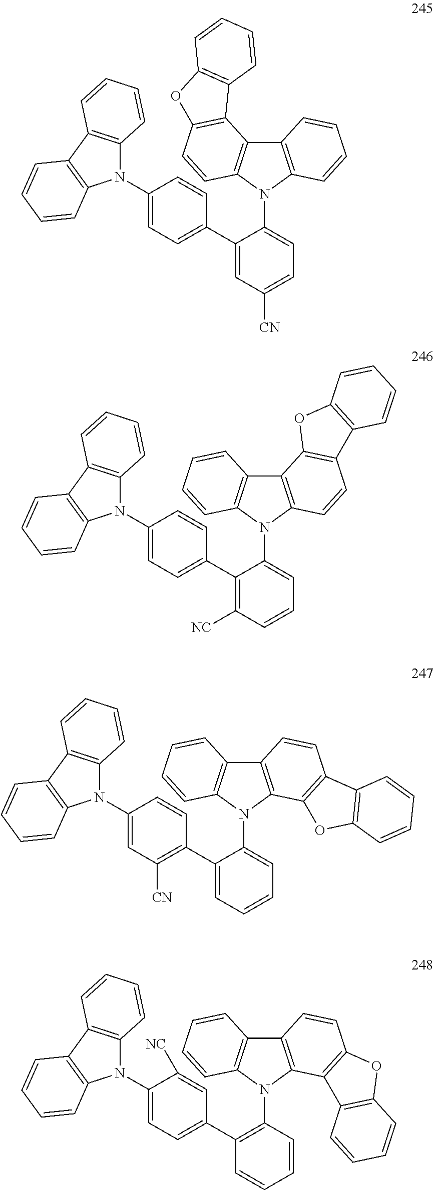 [SMILES: N#Cc1ccc(-n2c3ccccc3c3c4c(ccc32)oc2ccccc24)c(-c2ccc(-n3c4ccccc4c4ccccc43)cc2)c1 ; N#Cc1cccc(-n2c3ccccc3c3c4oc5ccccc5c4ccc32)c1-c1ccc(-n2c3ccccc3c3ccccc32)cc1 ; N#Cc1cc(-n2c3ccccc3c3ccccc32)ccc1-c1ccccc1-n1c2ccccc2c2ccc3c4ccccc4oc3c21 ; N#Cc1cc(-c2ccccc2-n2c3ccccc3c3ccc4oc5ccccc5c4c32)ccc1-n1c2ccccc2c2ccccc21]

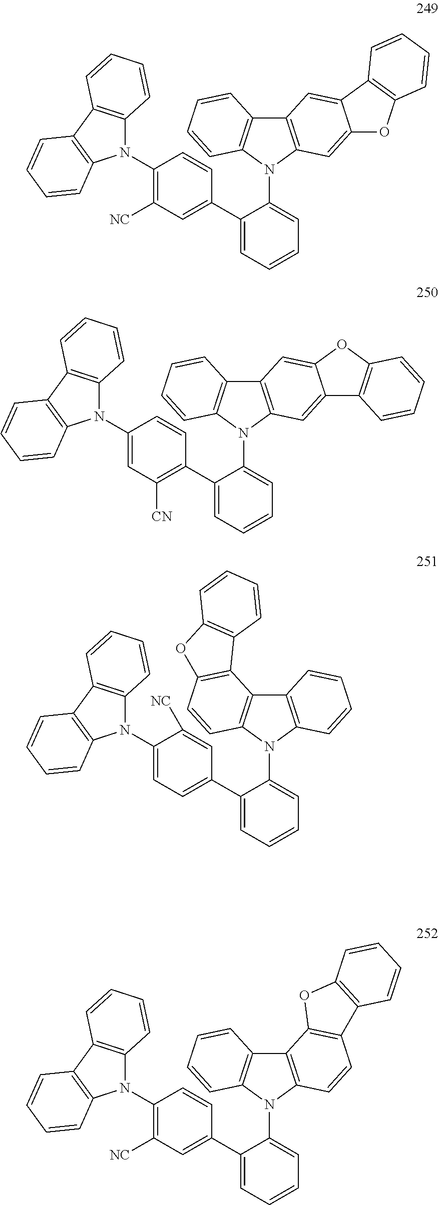

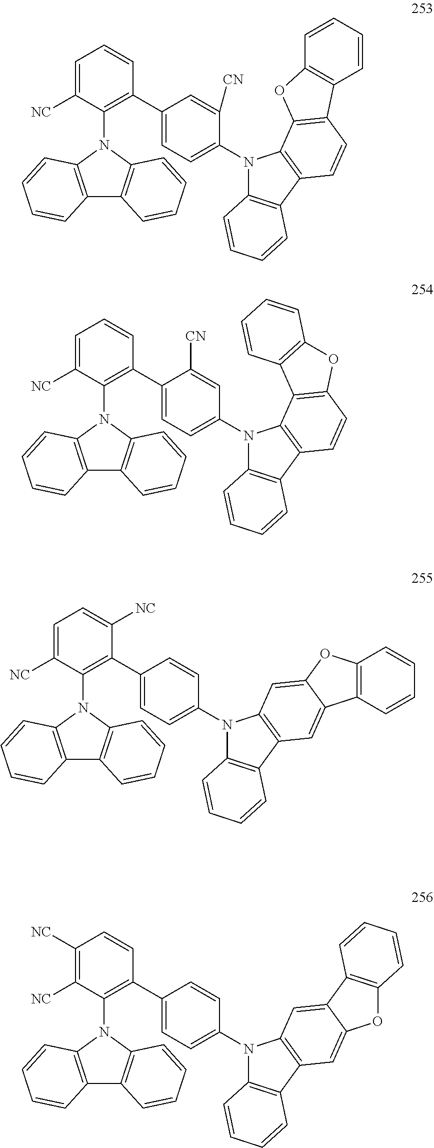

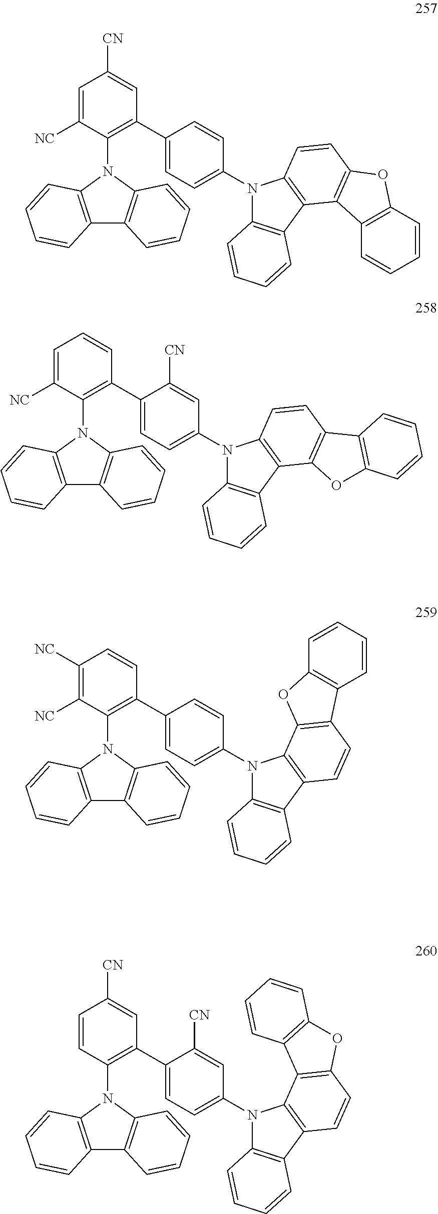 [SMILES: N#Cc1cc(C#N)c(-n2c3ccccc3c3ccccc32)c(-c2ccc(-n3c4ccccc4c4c5c(ccc43)oc3ccccc35)cc2)c1 ; N#Cc1cc(-n2c3ccccc3c3c4oc5ccccc5c4ccc32)ccc1-c1cccc(C#N)c1-n1c2ccccc2c2ccccc21 ; N#Cc1ccc(-c2ccc(-n3c4ccccc4c4ccc5c6ccccc6oc5c43)cc2)c(-n2c3ccccc3c3ccccc32)c1C#N ; N#Cc1ccc(-n2c3ccccc3c3ccccc32)c(-c2ccc(-n3c4ccccc4c4ccc5oc6ccccc6c5c43)cc2C#N)c1]

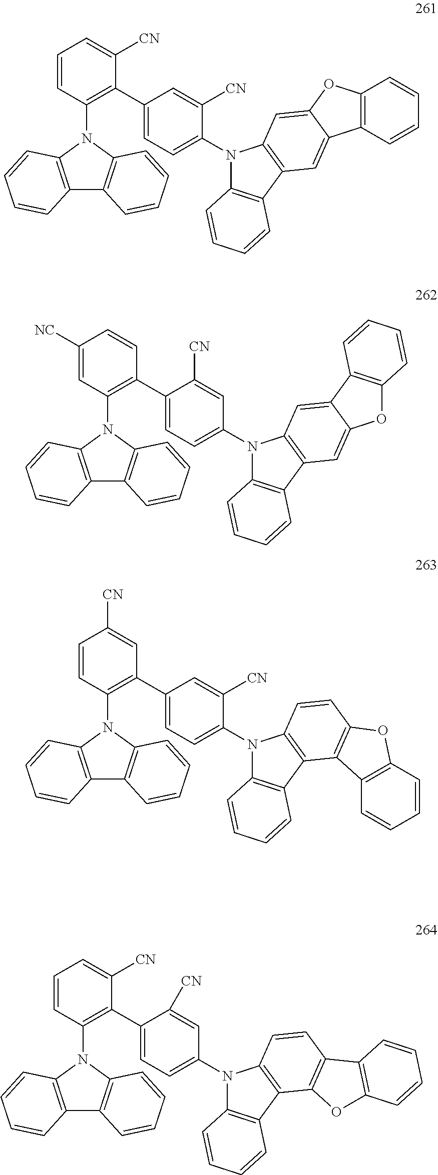 [SMILES: N#Cc1cc(-c2c(C#N)cccc2-n2c3ccccc3c3ccccc32)ccc1-n1c2ccccc2c2cc3c(cc21)oc1ccccc13 ; N#Cc1ccc(-c2ccc(-n3c4ccccc4c4cc5oc6ccccc6c5cc43)cc2C#N)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)c(C#N)c2)c1 ; N#Cc1cc(-n2c3ccccc3c3c4oc5ccccc5c4ccc32)ccc1-c1c(C#N)cccc1-n1c2ccccc2c2ccccc21]

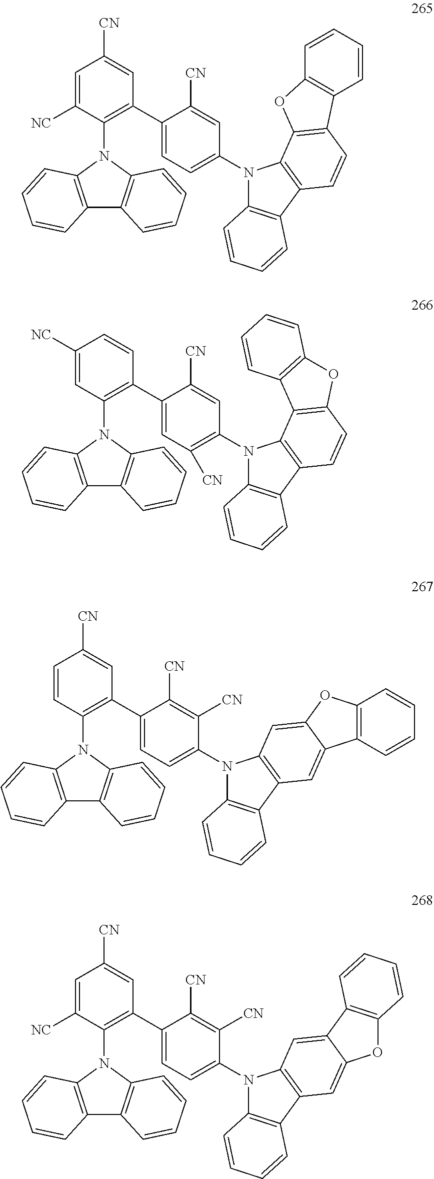

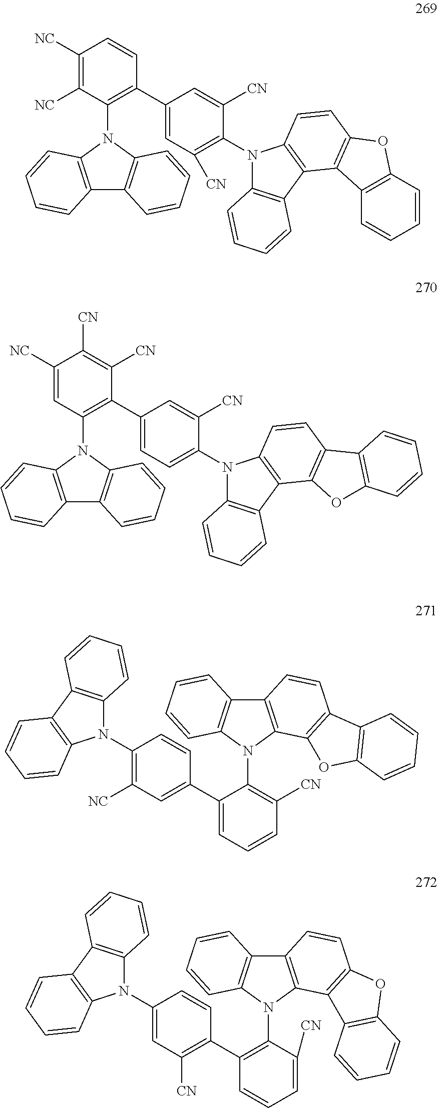 [SMILES: N#Cc1ccc(-c2cc(C#N)c(-n3c4ccccc4c4c5c(ccc43)oc3ccccc35)c(C#N)c2)c(-n2c3ccccc3c3ccccc32)c1C#N ; N#Cc1cc(-c2c(-n3c4ccccc4c4ccccc43)cc(C#N)c(C#N)c2C#N)ccc1-n1c2ccccc2c2c3oc4ccccc4c3ccc21 ; N#Cc1cc(-c2cccc(C#N)c2-n2c3ccccc3c3ccc4c5ccccc5oc4c32)ccc1-n1c2ccccc2c2ccccc21 ; N#Cc1cc(-n2c3ccccc3c3ccccc32)ccc1-c1cccc(C#N)c1-n1c2ccccc2c2ccc3oc4ccccc4c3c21]

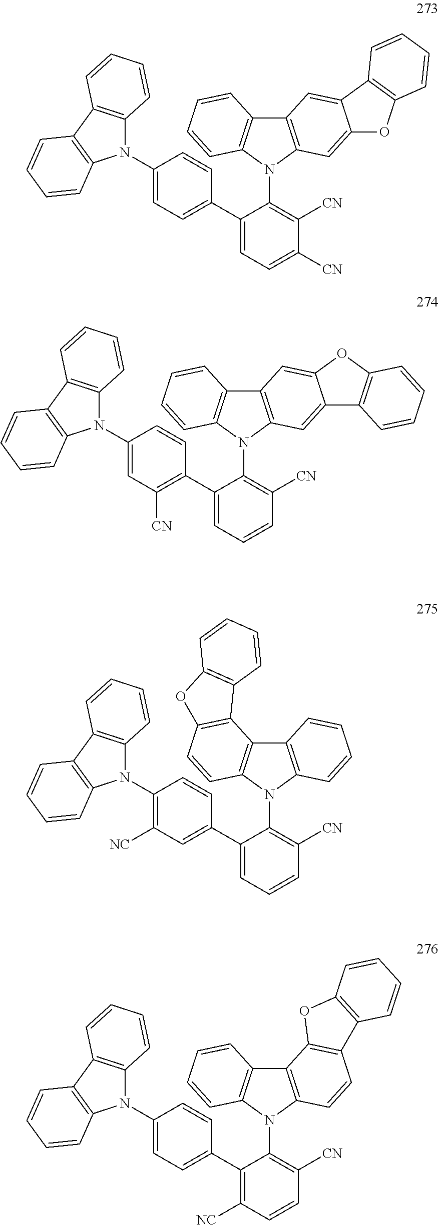 [SMILES: N#Cc1ccc(-c2ccc(-n3c4ccccc4c4ccccc43)cc2)c(-n2c3ccccc3c3cc4c(cc32)oc2ccccc24)c1C#N ; N#Cc1cc(-n2c3ccccc3c3ccccc32)ccc1-c1cccc(C#N)c1-n1c2ccccc2c2cc3oc4ccccc4c3cc21 ; N#Cc1cc(-c2cccc(C#N)c2-n2c3ccccc3c3c4c(ccc32)oc2ccccc24)ccc1-n1c2ccccc2c2ccccc21 ; N#Cc1ccc(C#N)c(-n2c3ccccc3c3c4oc5ccccc5c4ccc32)c1-c1ccc(-n2c3ccccc3c3ccccc32)cc1]

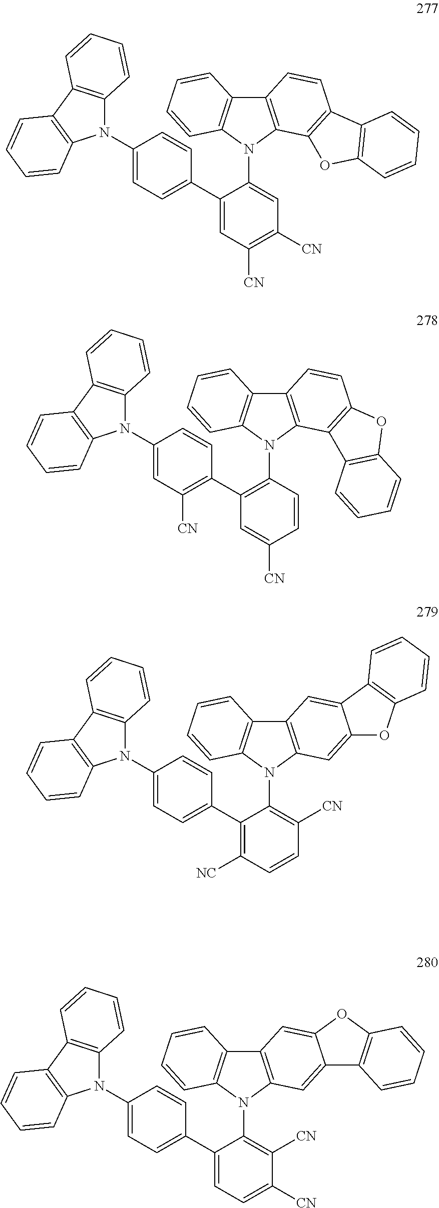 [SMILES: N#Cc1cc(-c2ccc(-n3c4ccccc4c4ccccc43)cc2)c(-n2c3ccccc3c3ccc4c5ccccc5oc4c32)cc1C#N ; N#Cc1ccc(-n2c3ccccc3c3ccc4oc5ccccc5c4c32)c(-c2ccc(-n3c4ccccc4c4ccccc43)cc2C#N)c1 ; N#Cc1ccc(C#N)c(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1C#N]

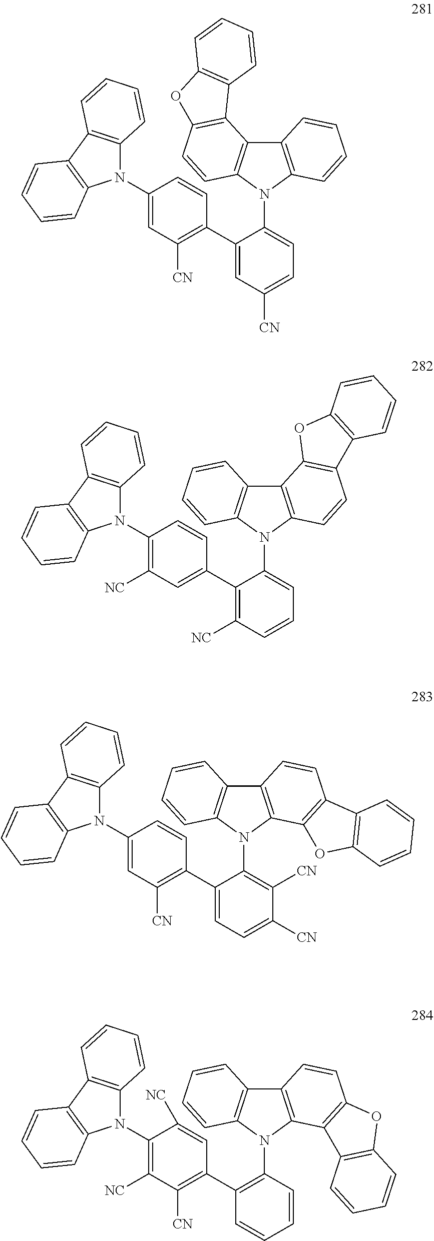 [SMILES: N#Cc1ccc(-n2c3ccccc3c3c4c(ccc32)oc2ccccc24)c(-c2ccc(-n3c4ccccc4c4ccccc43)cc2C#N)c1 ; N#Cc1cc(-c2c(C#N)cccc2-n2c3ccccc3c3c4oc5ccccc5c4ccc32)ccc1-n1c2ccccc2c2ccccc21 ; N#Cc1cc(-n2c3ccccc3c3ccccc32)ccc1-c1ccc(C#N)c(C#N)c1-n1c2ccccc2c2ccc3c4ccccc4oc3c21 ; N#Cc1cc(-c2ccccc2-n2c3ccccc3c3ccc4oc5ccccc5c4c32)c(C#N)c(C#N)c1-n1c2ccccc2c2ccccc21]

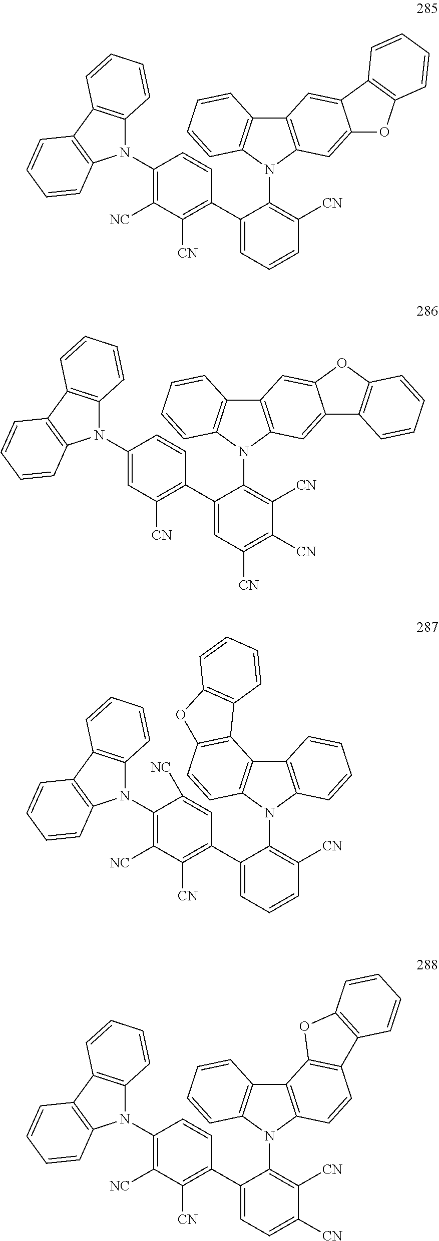 [SMILES: N#Cc1cccc(-c2ccc(-n3c4ccccc4c4ccccc43)c(C#N)c2C#N)c1-n1c2ccccc2c2cc3c(cc21)oc1ccccc13 ; N#Cc1cc(-n2c3ccccc3c3ccccc32)ccc1-c1cc(C#N)c(C#N)c(C#N)c1-n1c2ccccc2c2cc3oc4ccccc4c3cc21 ; N#Cc1cc(-c2cccc(C#N)c2-n2c3ccccc3c3c4c(ccc32)oc2ccccc24)c(C#N)c(C#N)c1-n1c2ccccc2c2ccccc21 ; N#Cc1ccc(-c2ccc(-n3c4ccccc4c4ccccc43)c(C#N)c2C#N)c(-n2c3ccccc3c3c4oc5ccccc5c4ccc32)c1C#N]

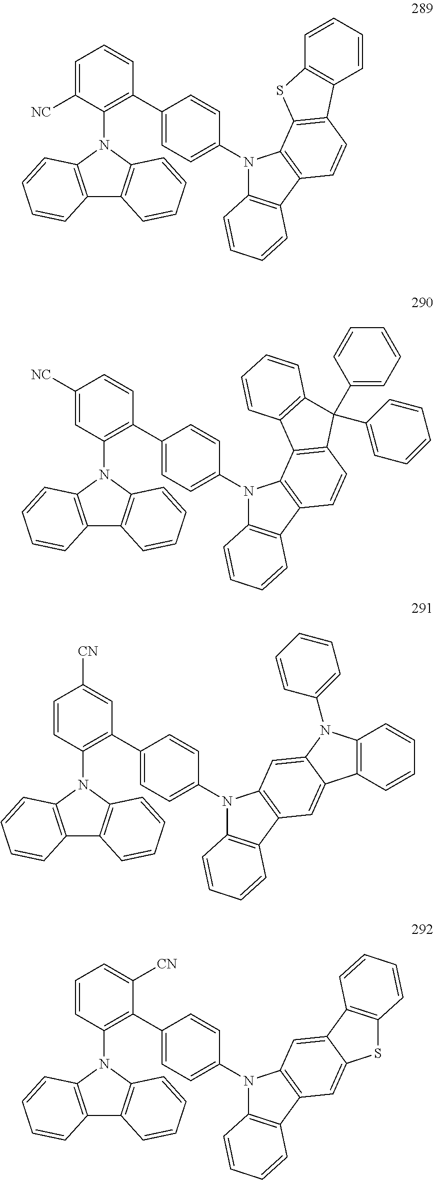 [SMILES: N#Cc1cccc(-c2ccc(-n3c4ccccc4c4ccc5c6ccccc6sc5c43)cc2)c1-n1c2ccccc2c2ccccc21 ; N#Cc1ccc(-c2ccc(-n3c4ccccc4c4ccc5c(c43)-c3ccccc3C5(c3ccccc3)c3ccccc3)cc2)c(-n2c3ccccc3c3ccccc32)c1 ; N#Cc1ccc(-n2c3ccccc3c3ccccc32)c(-c2ccc(-n3c4ccccc4c4cc5c6ccccc6n(-c6ccccc6)c5cc43)cc2)c1 ; N#Cc1cccc(-n2c3ccccc3c3ccccc32)c1-c1ccc(-n2c3ccccc3c3cc4sc5ccccc5c4cc32)cc1]

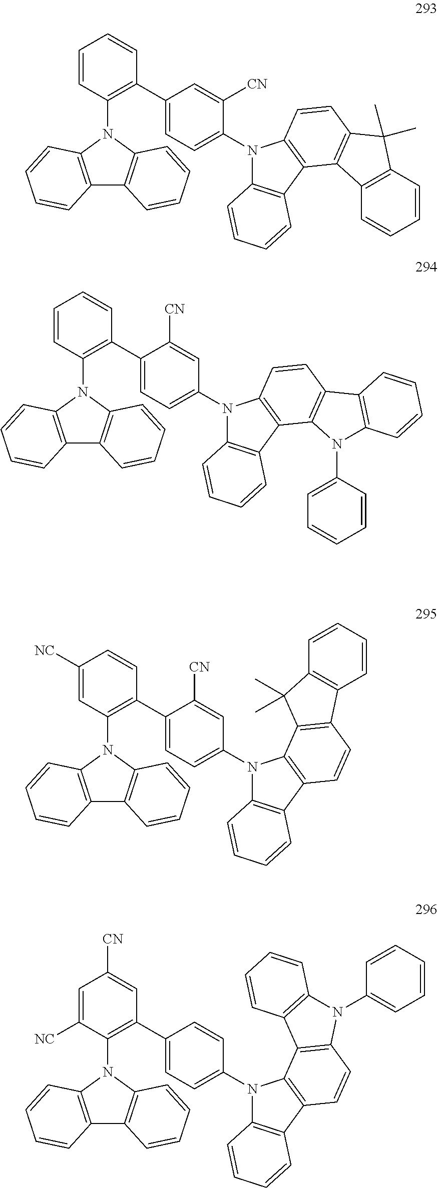 [SMILES: CC1(C)c2ccccc2-c2c1ccc1c2c2ccccc2n1-c1ccc(-c2ccccc2-n2c3ccccc3c3ccccc32)cc1C#N ; N#Cc1cc(-n2c3ccccc3c3c2ccc2c4ccccc4n(-c4ccccc4)c23)ccc1-c1ccccc1-n1c2ccccc2c2ccccc21 ; CC1(C)c2ccccc2-c2ccc3c4ccccc4n(-c4ccc(-c5ccc(C#N)cc5-n5c6ccccc6c6ccccc65)c(C#N)c4)c3c21 ; N#Cc1cc(C#N)c(-n2c3ccccc3c3ccccc32)c(-c2ccc(-n3c4ccccc4c4ccc5c(c6ccccc6n5-c5ccccc5)c43)cc2)c1]

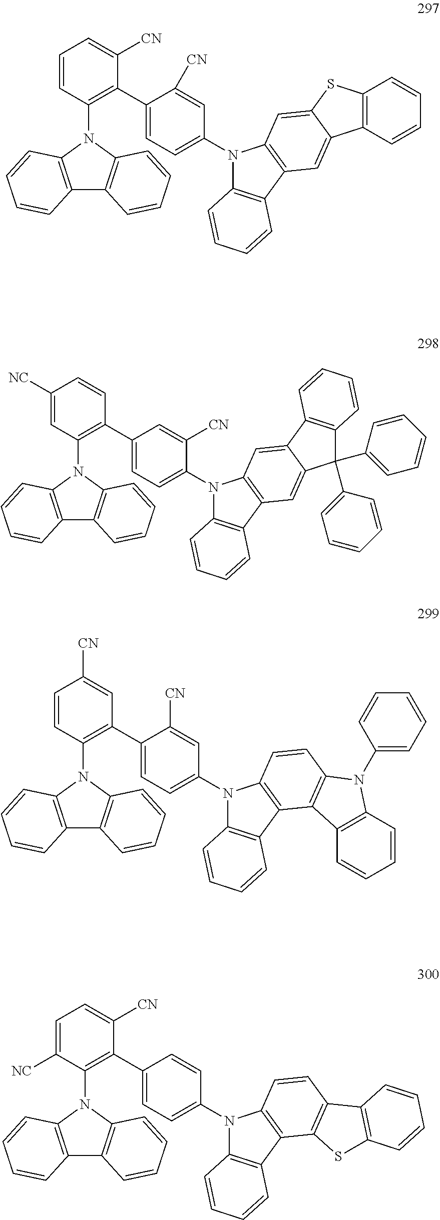 [SMILES: N#Cc1cc(-n2c3ccccc3c3cc4c(cc32)sc2ccccc24)ccc1-c1c(C#N)cccc1-n1c2ccccc2c2ccccc21 ; N#Cc1ccc(-c2ccc(-n3c4ccccc4c4cc5c(cc43)-c3ccccc3C5(c3ccccc3)c3ccccc3)c(C#N)c2)c(-n2c3ccccc3c3ccccc32)c1 ; N#Cc1ccc(-n2c3ccccc3c3ccccc32)c(-c2ccc(-n3c4ccccc4c4c5c6ccccc6n(-c6ccccc6)c5ccc43)cc2C#N)c1 ; N#Cc1ccc(C#N)c(-n2c3ccccc3c3ccccc32)c1-c1ccc(-n2c3ccccc3c3c4sc5ccccc5c4ccc32)cc1]

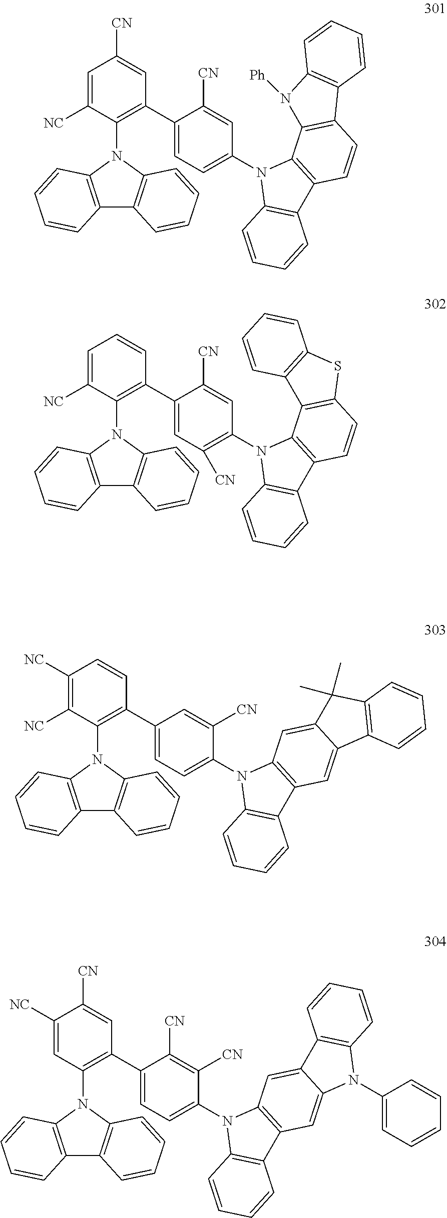 [SMILES: N#Cc1cc(C#N)c(-n2c3ccccc3c3ccccc32)c(-c2ccc(-n3c4ccccc4c4ccc5c6ccccc6n(-c6ccccc6)c5c43)cc2C#N)c1 ; N#Cc1cc(-n2c3ccccc3c3ccc4sc5ccccc5c4c32)c(C#N)cc1-c1cccc(C#N)c1-n1c2ccccc2c2ccccc21 ; CC1(C)c2ccccc2-c2cc3c4ccccc4n(-c4ccc(-c5ccc(C#N)c(C#N)c5-n5c6ccccc6c6ccccc65)cc4C#N)c3cc21 ; N#Cc1cc(-c2ccc(-n3c4ccccc4c4cc5c(cc43)c3ccccc3n5-c3ccccc3)c(C#N)c2C#N)c(-n2c3ccccc3c3ccccc32)cc1C#N]

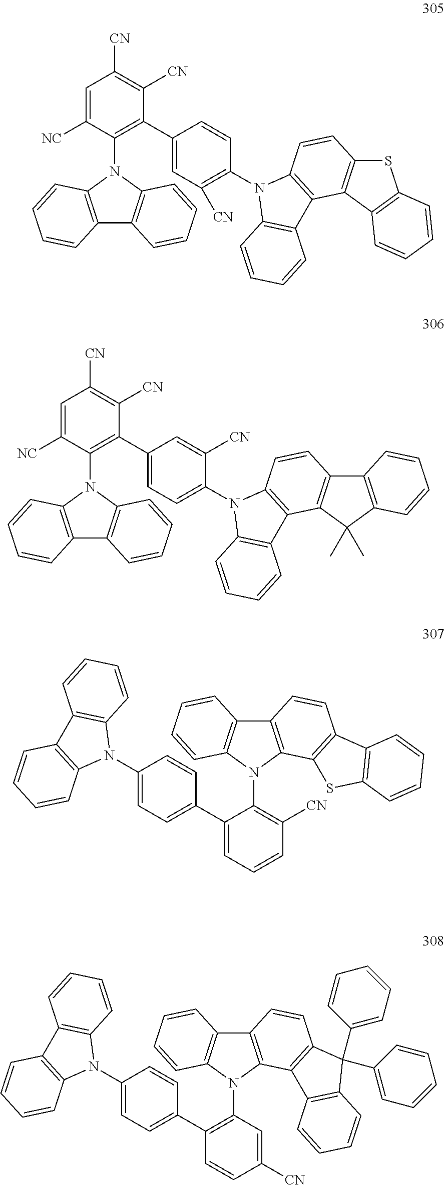 [SMILES: N#Cc1cc(-c2c(C#N)c(C#N)cc(C#N)c2-n2c3ccccc3c3ccccc32)ccc1-n1c2ccccc2c2c3c(ccc21)sc1ccccc13 ; CC1(C)c2ccccc2-c2ccc3c(c21)c1ccccc1n3-c1ccc(-c2c(C#N)c(C#N)cc(C#N)c2-n2c3ccccc3c3ccccc32)cc1C#N ; N#Cc1cccc(-c2ccc(-n3c4ccccc4c4ccccc43)cc2)c1-n1c2ccccc2c2ccc3c4ccccc4sc3c21 ; N#Cc1ccc(-c2ccc(-n3c4ccccc4c4ccccc43)cc2)c(-n2c3ccccc3c3ccc4c(c32)-c2ccccc2C4(c2ccccc2)c2ccccc2)c1]

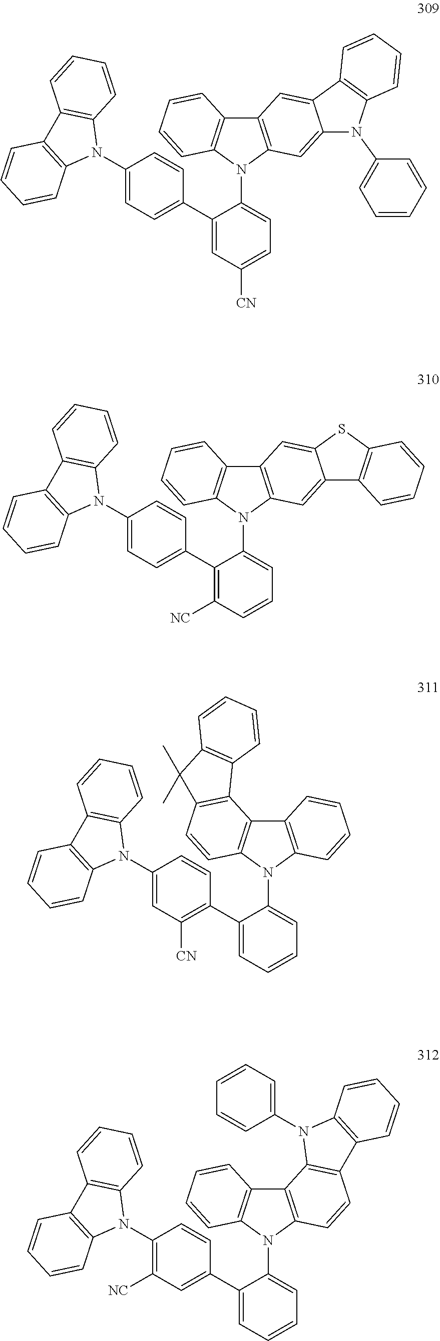 [SMILES: N#Cc1ccc(-n2c3ccccc3c3cc4c5ccccc5n(-c5ccccc5)c4cc32)c(-c2ccc(-n3c4ccccc4c4ccccc43)cc2)c1 ; N#Cc1cccc(-n2c3ccccc3c3cc4sc5ccccc5c4cc32)c1-c1ccc(-n2c3ccccc3c3ccccc32)cc1 ; CC1(C)c2ccccc2-c2c1ccc1c2c2ccccc2n1-c1ccccc1-c1ccc(-n2c3ccccc3c3ccccc32)cc1C#N ; N#Cc1cc(-c2ccccc2-n2c3ccccc3c3c2ccc2c4ccccc4n(-c4ccccc4)c23)ccc1-n1c2ccccc2c2ccccc21]

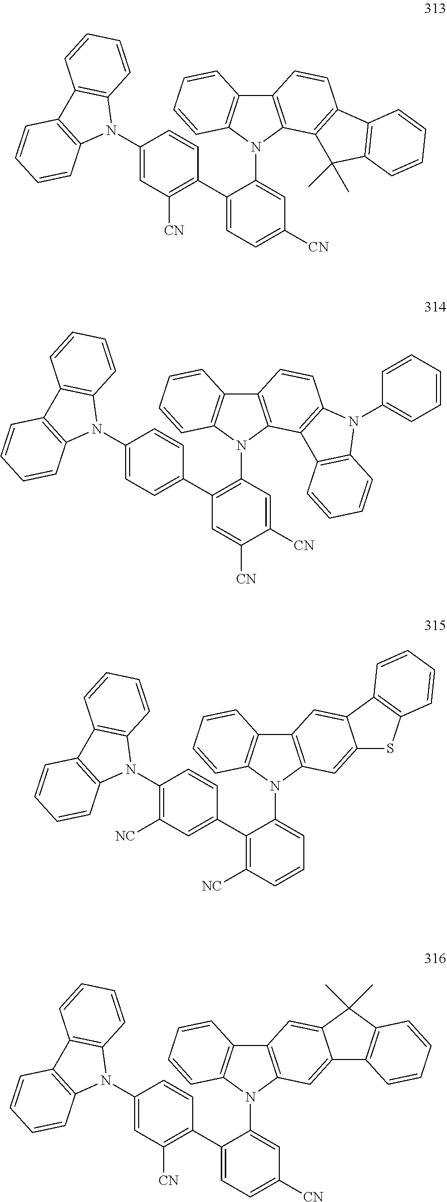 [SMILES: CC1(C)c2ccccc2-c2ccc3c4ccccc4n(-c4cc(C#N)ccc4-c4ccc(-n5c6ccccc6c6ccccc65)cc4C#N)c3c21 ; N#Cc1cc(-c2ccc(-n3c4ccccc4c4ccccc43)cc2)c(-n2c3ccccc3c3ccc4c(c5ccccc5n4-c4ccccc4)c32)cc1C#N ; N#Cc1cc(-c2c(C#N)cccc2-n2c3ccccc3c3cc4c(cc32)sc2ccccc24)ccc1-n1c2ccccc2c2ccccc21 ; CC1(C)c2ccccc2-c2cc3c(cc21)c1ccccc1n3-c1cc(C#N)ccc1-c1ccc(-n2c3ccccc3c3ccccc32)cc1C#N]

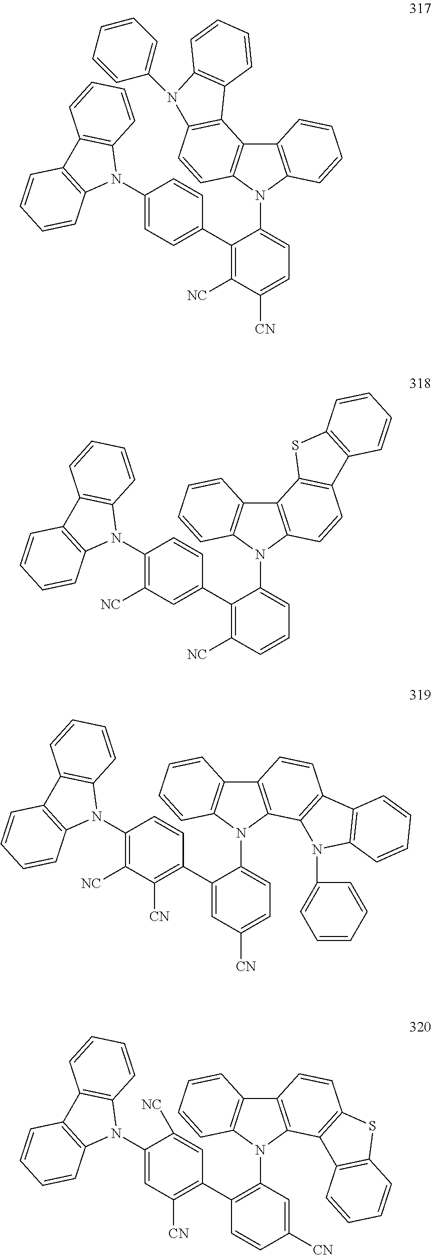 [SMILES: N#Cc1ccc(-n2c3ccccc3c3c4c5ccccc5n(-c5ccccc5)c4ccc32)c(-c2ccc(-n3c4ccccc4c4ccccc43)cc2)c1C#N ; N#Cc1cc(-c2c(C#N)cccc2-n2c3ccccc3c3c4sc5ccccc5c4ccc32)ccc1-n1c2ccccc2c2ccccc21 ; N#Cc1ccc(-n2c3ccccc3c3ccc4c5ccccc5n(-c5ccccc5)c4c32)c(-c2ccc(-n3c4ccccc4c4ccccc43)c(C#N)c2C#N)c1 ; N#Cc1ccc(-c2cc(C#N)c(-n3c4ccccc4c4ccccc43)cc2C#N)c(-n2c3ccccc3c3ccc4sc5ccccc5c4c32)c1]

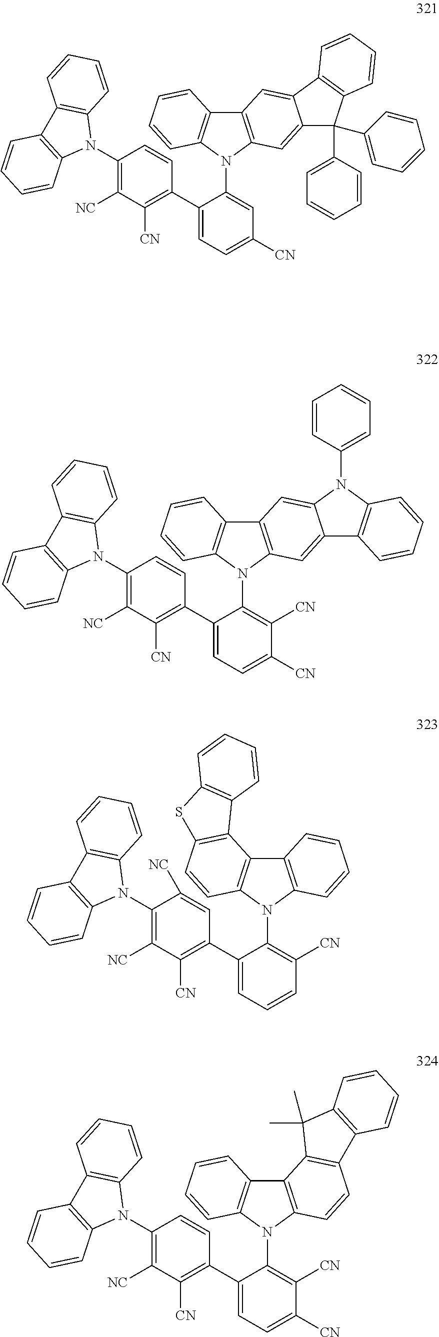 [SMILES: N#Cc1ccc(-c2ccc(-n3c4ccccc4c4ccccc43)c(C#N)c2C#N)c(-n2c3ccccc3c3cc4c(cc32)C(c2ccccc2)(c2ccccc2)c2ccccc2-4)c1 ; N#Cc1ccc(-c2ccc(-n3c4ccccc4c4ccccc43)c(C#N)c2C#N)c(-n2c3ccccc3c3cc4c(cc32)c2ccccc2n4-c2ccccc2)c1C#N ; N#Cc1cc(-c2cccc(C#N)c2-n2c3ccccc3c3c4c(ccc32)sc2ccccc24)c(C#N)c(C#N)c1-n1c2ccccc2c2ccccc21 ; CC1(C)c2ccccc2-c2ccc3c(c21)c1ccccc1n3-c1c(-c2ccc(-n3c4ccccc4c4ccccc43)c(C#N)c2C#N)ccc(C#N)c1C#N]

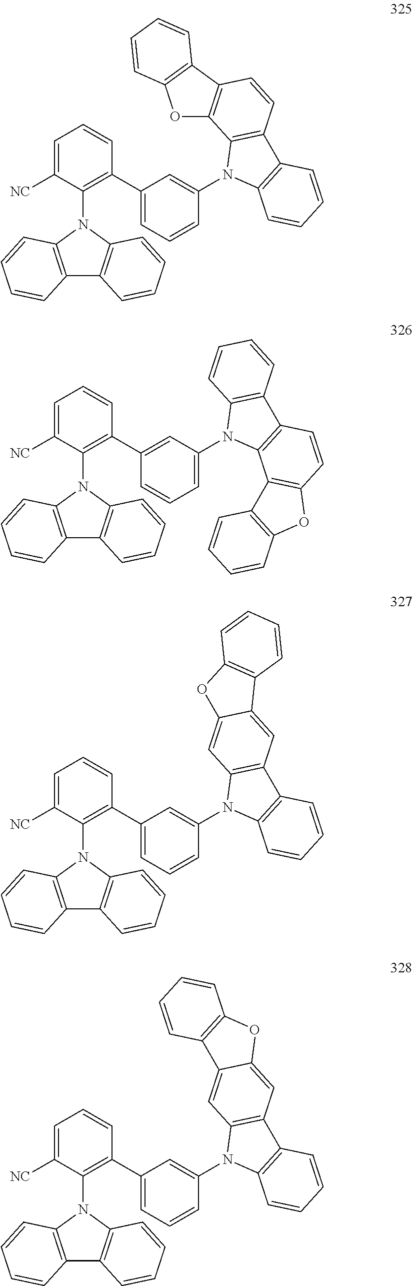

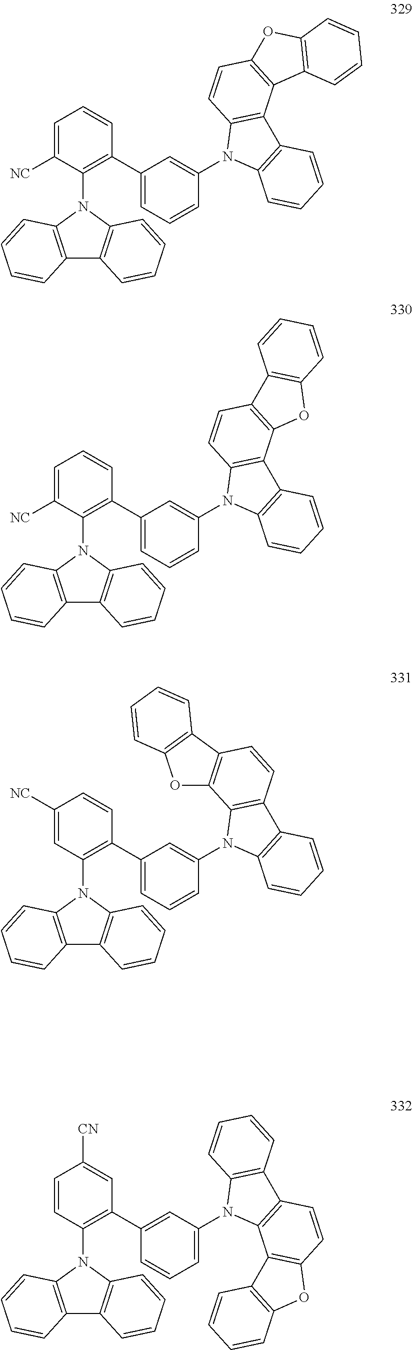 [SMILES: N#Cc1cccc(-c2cccc(-n3c4ccccc4c4c5c(ccc43)oc3ccccc35)c2)c1-n1c2ccccc2c2ccccc21 ; N#Cc1cccc(-c2cccc(-n3c4ccccc4c4c5oc6ccccc6c5ccc43)c2)c1-n1c2ccccc2c2ccccc21 ; N#Cc1ccc(-c2cccc(-n3c4ccccc4c4ccc5c6ccccc6oc5c43)c2)c(-n2c3ccccc3c3ccccc32)c1 ; N#Cc1ccc(-n2c3ccccc3c3ccccc32)c(-c2cccc(-n3c4ccccc4c4ccc5oc6ccccc6c5c43)c2)c1]

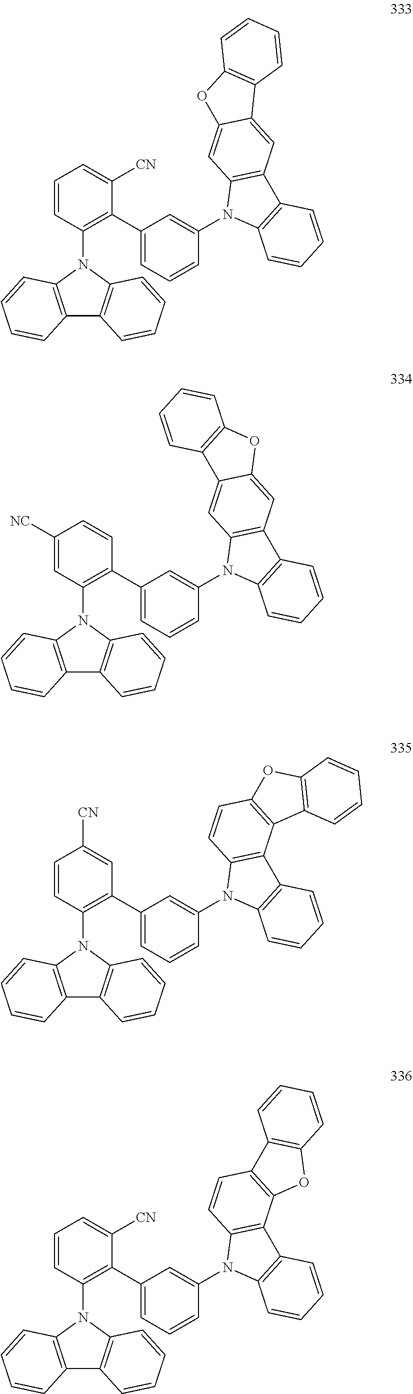 [SMILES: N#Cc1cccc(-n2c3ccccc3c3ccccc32)c1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)c(-n2c3ccccc3c3ccccc32)c1 ; N#Cc1ccc(-n2c3ccccc3c3ccccc32)c(-c2cccc(-n3c4ccccc4c4c5c(ccc43)oc3ccccc35)c2)c1 ; N#Cc1cccc(-n2c3ccccc3c3ccccc32)c1-c1cccc(-n2c3ccccc3c3c4oc5ccccc5c4ccc32)c1]

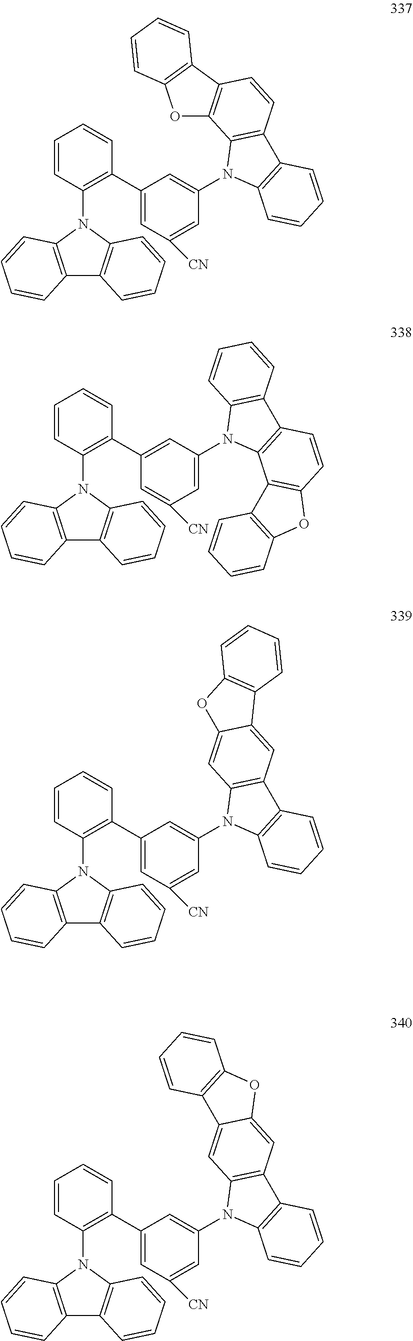 [SMILES: N#Cc1cc(-c2ccccc2-n2c3ccccc3c3ccccc32)cc(-n2c3ccccc3c3ccc4c5ccccc5oc4c32)c1 ; N#Cc1cc(-c2ccccc2-n2c3ccccc3c3ccccc32)cc(-n2c3ccccc3c3ccc4oc5ccccc5c4c32)c1 ; N#Cc1cc(-c2ccccc2-n2c3ccccc3c3ccccc32)cc(-n2c3ccccc3c3cc4c(cc32)oc2ccccc24)c1 ; N#Cc1cc(-c2ccccc2-n2c3ccccc3c3ccccc32)cc(-n2c3ccccc3c3cc4oc5ccccc5c4cc32)c1]

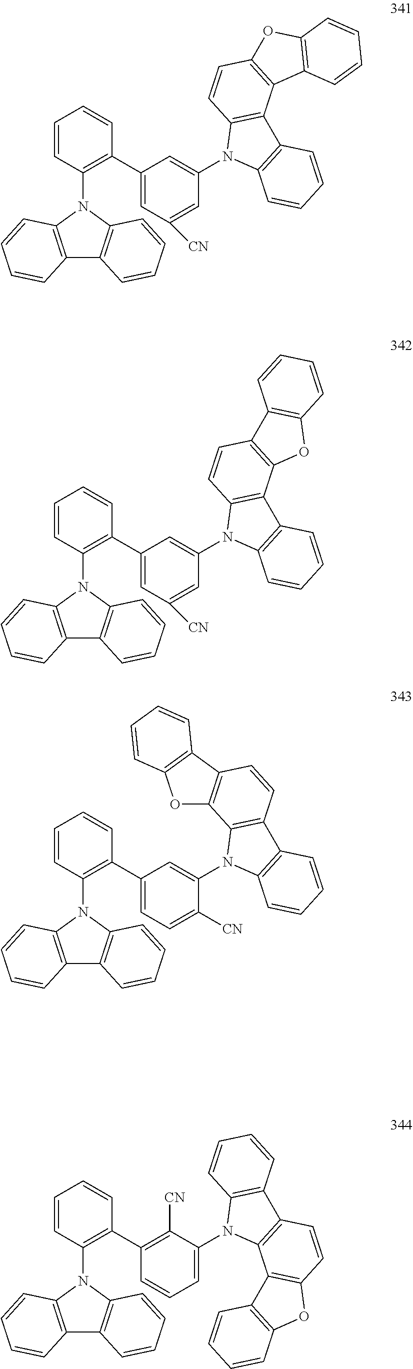 [SMILES: N#Cc1cc(-c2ccccc2-n2c3ccccc3c3ccccc32)cc(-n2c3ccccc3c3c4c(ccc32)oc2ccccc24)c1 ; N#Cc1cc(-c2ccccc2-n2c3ccccc3c3ccccc32)cc(-n2c3ccccc3c3c4oc5ccccc5c4ccc32)c1 ; N#Cc1ccc(-c2ccccc2-n2c3ccccc3c3ccccc32)cc1-n1c2ccccc2c2ccc3c4ccccc4oc3c21 ; N#Cc1c(-c2ccccc2-n2c3ccccc3c3ccccc32)cccc1-n1c2ccccc2c2ccc3oc4ccccc4c3c21]

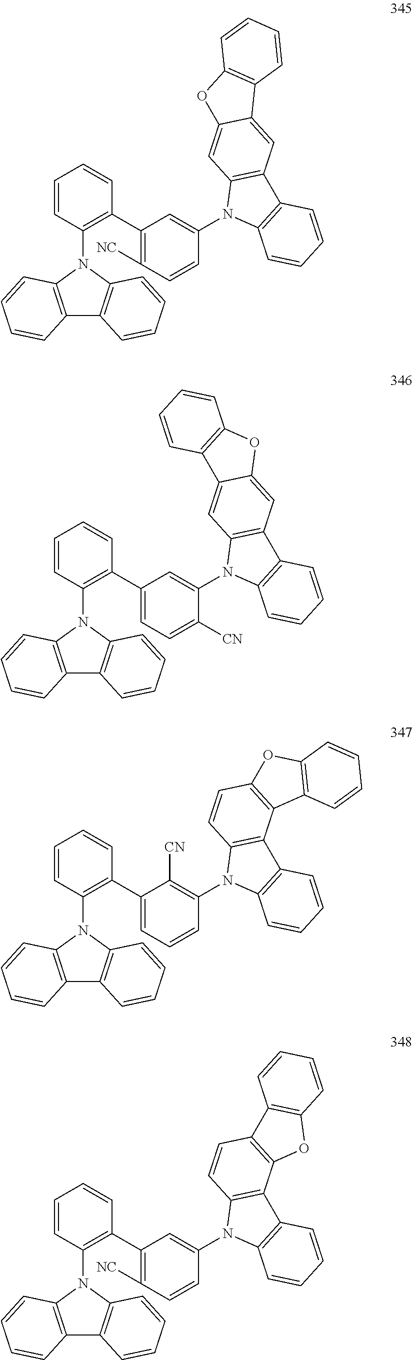 [SMILES: N#Cc1ccc(-n2c3ccccc3c3cc4c(cc32)oc2ccccc24)cc1-c1ccccc1-n1c2ccccc2c2ccccc21 ; N#Cc1ccc(-c2ccccc2-n2c3ccccc3c3ccccc32)cc1-n1c2ccccc2c2cc3oc4ccccc4c3cc21 ; N#Cc1c(-c2ccccc2-n2c3ccccc3c3ccccc32)cccc1-n1c2ccccc2c2c3c(ccc21)oc1ccccc13 ; N#Cc1ccc(-n2c3ccccc3c3c4oc5ccccc5c4ccc32)cc1-c1ccccc1-n1c2ccccc2c2ccccc21]

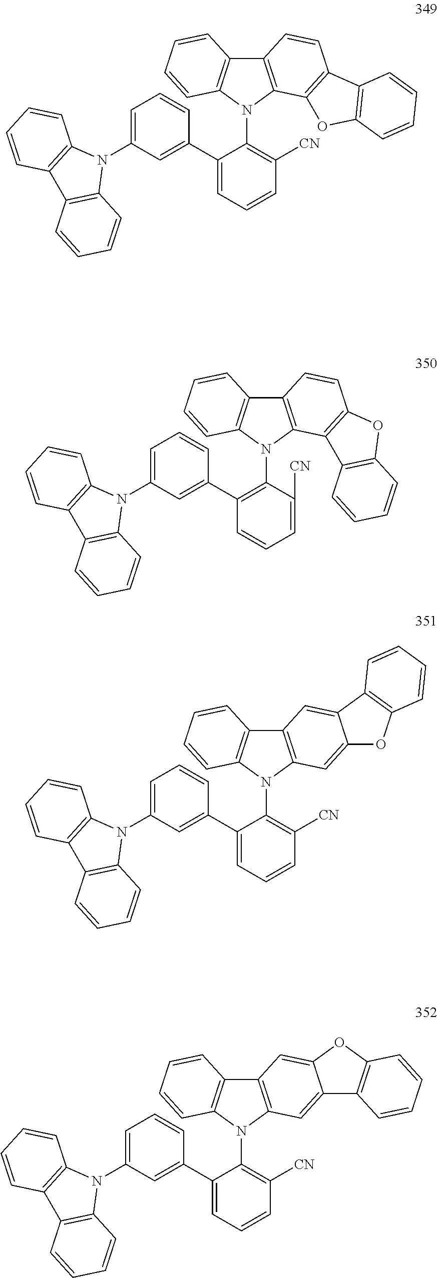 [SMILES: N#Cc1cccc(-c2cccc(-n3c4ccccc4c4ccccc43)c2)c1-n1c2ccccc2c2ccc3c4ccccc4oc3c21 ; N#Cc1cccc(-c2cccc(-n3c4ccccc4c4ccccc43)c2)c1-n1c2ccccc2c2ccc3oc4ccccc4c3c21 ; N#Cc1cccc(-c2cccc(-n3c4ccccc4c4ccccc43)c2)c1-n1c2ccccc2c2cc3c(cc21)oc1ccccc13 ; N#Cc1cccc(-c2cccc(-n3c4ccccc4c4ccccc43)c2)c1-n1c2ccccc2c2cc3oc4ccccc4c3cc21]

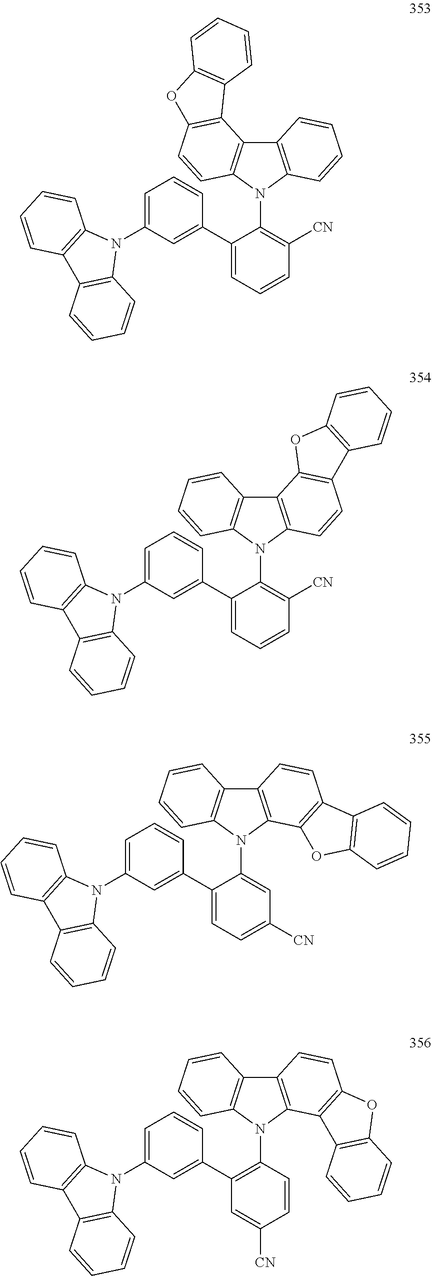 [SMILES: N#Cc1cccc(-c2cccc(-n3c4ccccc4c4ccccc43)c2)c1-n1c2ccccc2c2c3c(ccc21)oc1ccccc13 ; N#Cc1cccc(-c2cccc(-n3c4ccccc4c4ccccc43)c2)c1-n1c2ccccc2c2c3oc4ccccc4c3ccc21 ; N#Cc1ccc(-c2cccc(-n3c4ccccc4c4ccccc43)c2)c(-n2c3ccccc3c3ccc4c5ccccc5oc4c32)c1 ; N#Cc1ccc(-n2c3ccccc3c3ccc4oc5ccccc5c4c32)c(-c2cccc(-n3c4ccccc4c4ccccc43)c2)c1]

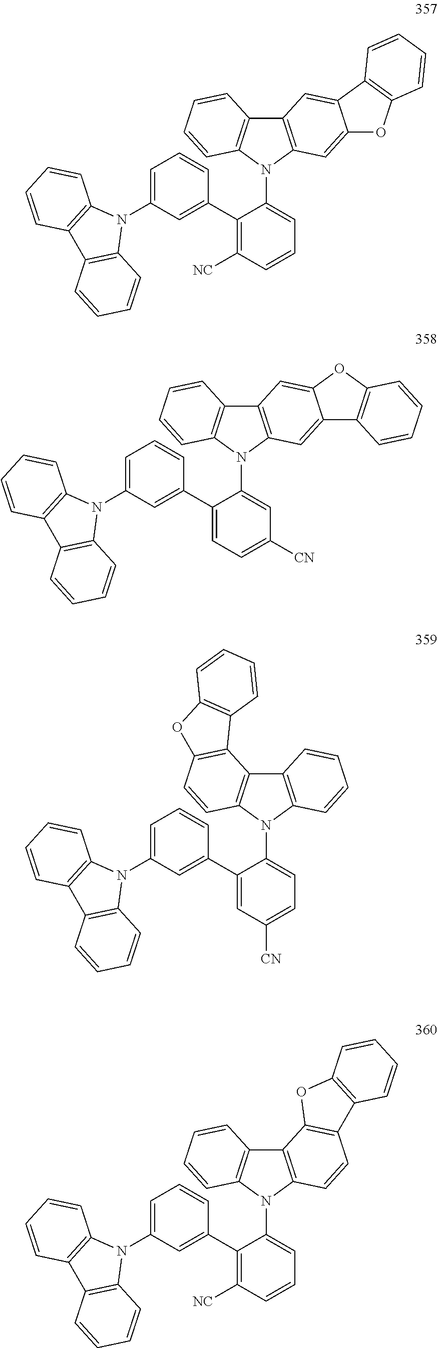 [SMILES: N#Cc1cccc(-n2c3ccccc3c3cc4c(cc32)oc2ccccc24)c1-c1cccc(-n2c3ccccc3c3ccccc32)c1 ; N#Cc1ccc(-c2cccc(-n3c4ccccc4c4ccccc43)c2)c(-n2c3ccccc3c3cc4oc5ccccc5c4cc32)c1 ; N#Cc1ccc(-n2c3ccccc3c3c4c(ccc32)oc2ccccc24)c(-c2cccc(-n3c4ccccc4c4ccccc43)c2)c1 ; N#Cc1cccc(-n2c3ccccc3c3c4oc5ccccc5c4ccc32)c1-c1cccc(-n2c3ccccc3c3ccccc32)c1]

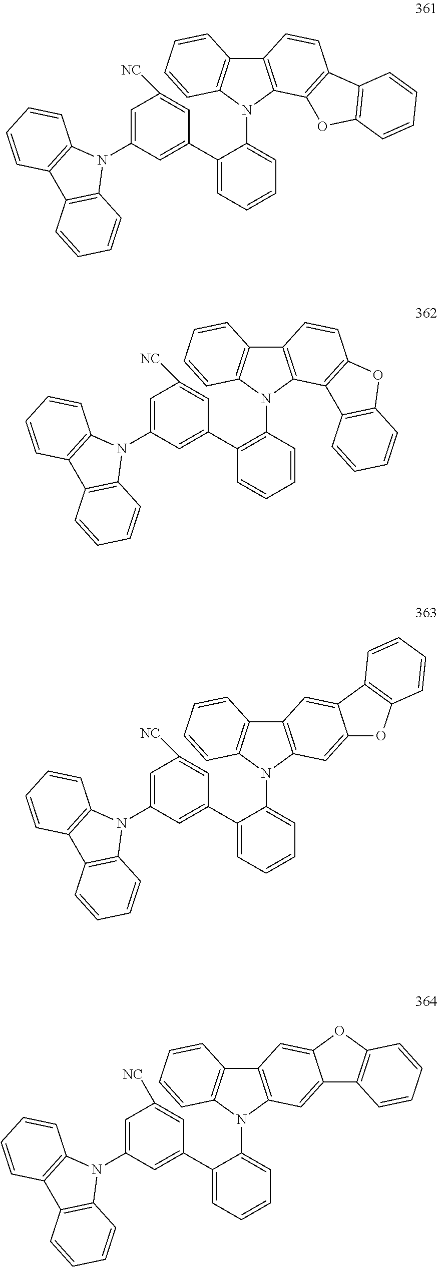 [SMILES: N#Cc1cc(-c2ccccc2-n2c3ccccc3c3ccc4c5ccccc5oc4c32)cc(-n2c3ccccc3c3ccccc32)c1 ; N#Cc1cc(-c2ccccc2-n2c3ccccc3c3ccc4oc5ccccc5c4c32)cc(-n2c3ccccc3c3ccccc32)c1 ; N#Cc1cc(-c2ccccc2-n2c3ccccc3c3cc4c(cc32)oc2ccccc24)cc(-n2c3ccccc3c3ccccc32)c1 ; N#Cc1cc(-c2ccccc2-n2c3ccccc3c3cc4oc5ccccc5c4cc32)cc(-n2c3ccccc3c3ccccc32)c1]

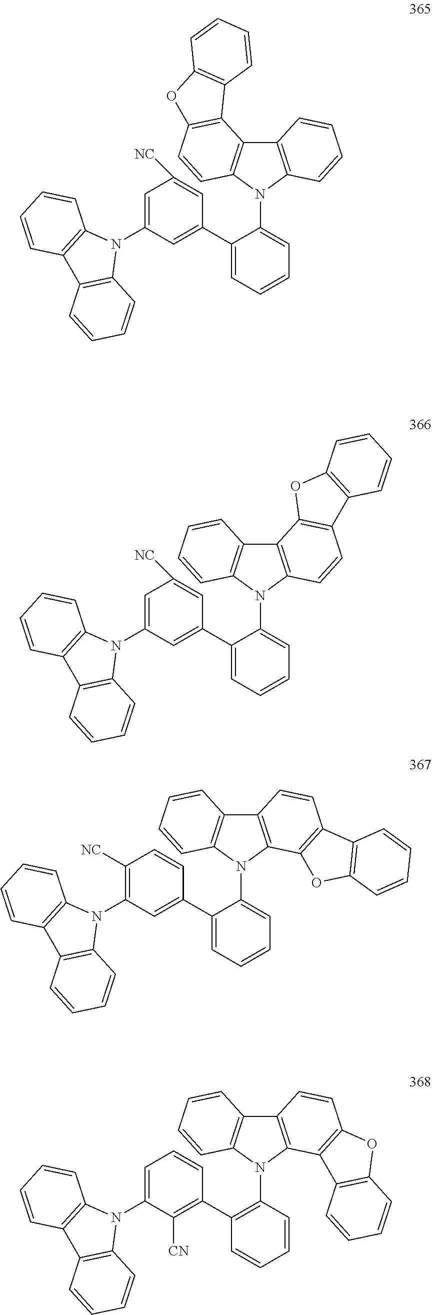 [SMILES: N#Cc1cc(-c2ccccc2-n2c3ccccc3c3c4c(ccc32)oc2ccccc24)cc(-n2c3ccccc3c3ccccc32)c1 ; N#Cc1cc(-c2ccccc2-n2c3ccccc3c3c4oc5ccccc5c4ccc32)cc(-n2c3ccccc3c3ccccc32)c1 ; N#Cc1ccc(-c2ccccc2-n2c3ccccc3c3ccc4c5ccccc5oc4c32)cc1-n1c2ccccc2c2ccccc21 ; N#Cc1c(-c2ccccc2-n2c3ccccc3c3ccc4oc5ccccc5c4c32)cccc1-n1c2ccccc2c2ccccc21]

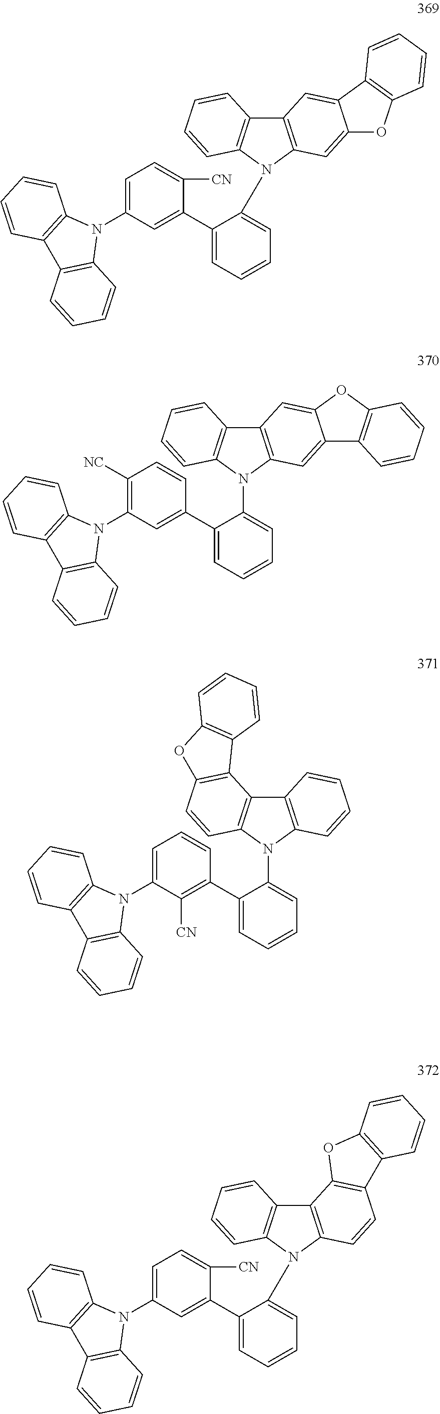 [SMILES: N#Cc1ccc(-n2c3ccccc3c3ccccc32)cc1-c1ccccc1-n1c2ccccc2c2cc3c(cc21)oc1ccccc13 ; N#Cc1ccc(-c2ccccc2-n2c3ccccc3c3cc4oc5ccccc5c4cc32)cc1-n1c2ccccc2c2ccccc21 ; N#Cc1c(-c2ccccc2-n2c3ccccc3c3c4c(ccc32)oc2ccccc24)cccc1-n1c2ccccc2c2ccccc21 ; N#Cc1ccc(-n2c3ccccc3c3ccccc32)cc1-c1ccccc1-n1c2ccccc2c2c3oc4ccccc4c3ccc21]

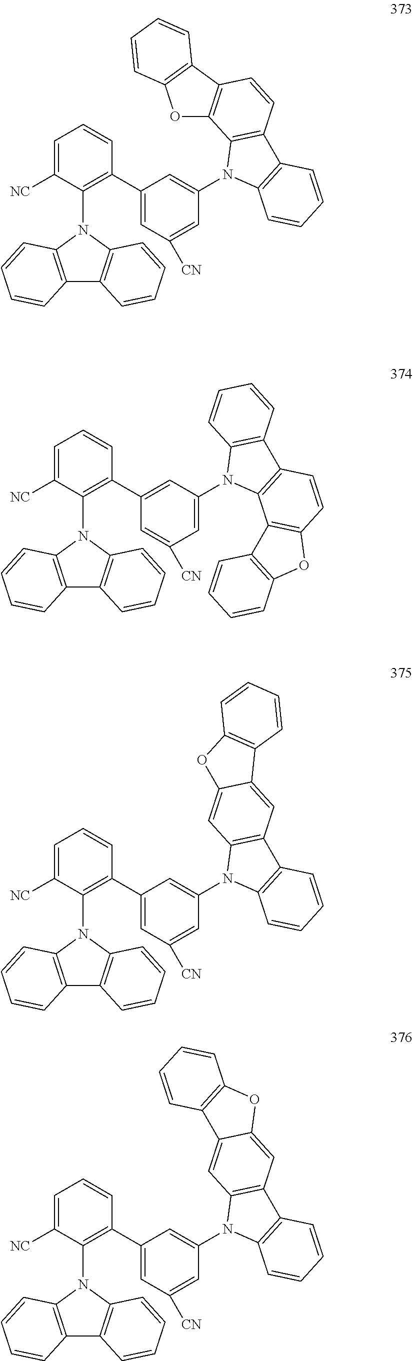 [SMILES: N#Cc1cc(-c2cccc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)cc(-n2c3ccccc3c3ccc4oc5ccccc5c4c32)c1 ; N#Cc1cc(-c2cccc(C#N)c2-n2c3ccccc3c3ccccc32)cc(-n2c3ccccc3c3cc4c(cc32)oc2ccccc24)c1 ; N#Cc1cc(-c2cccc(C#N)c2-n2c3ccccc3c3ccccc32)cc(-n2c3ccccc3c3cc4oc5ccccc5c4cc32)c1]

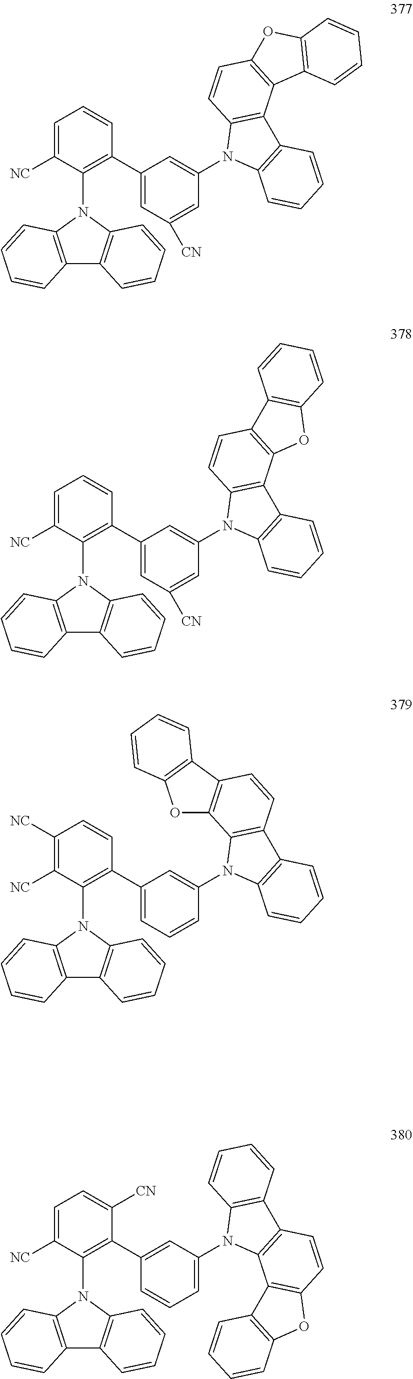 [SMILES: N#Cc1cc(-c2cccc(C#N)c2-n2c3ccccc3c3ccccc32)cc(-n2c3ccccc3c3c4c(ccc32)oc2ccccc24)c1 ; N#Cc1cc(-c2cccc(C#N)c2-n2c3ccccc3c3ccccc32)cc(-n2c3ccccc3c3c4oc5ccccc5c4ccc32)c1 ; N#Cc1ccc(-c2cccc(-n3c4ccccc4c4ccc5c6ccccc6oc5c43)c2)c(-n2c3ccccc3c3ccccc32)c1C#N ; N#Cc1ccc(C#N)c(-n2c3ccccc3c3ccccc32)c1-c1cccc(-n2c3ccccc3c3ccc4oc5ccccc5c4c32)c1]

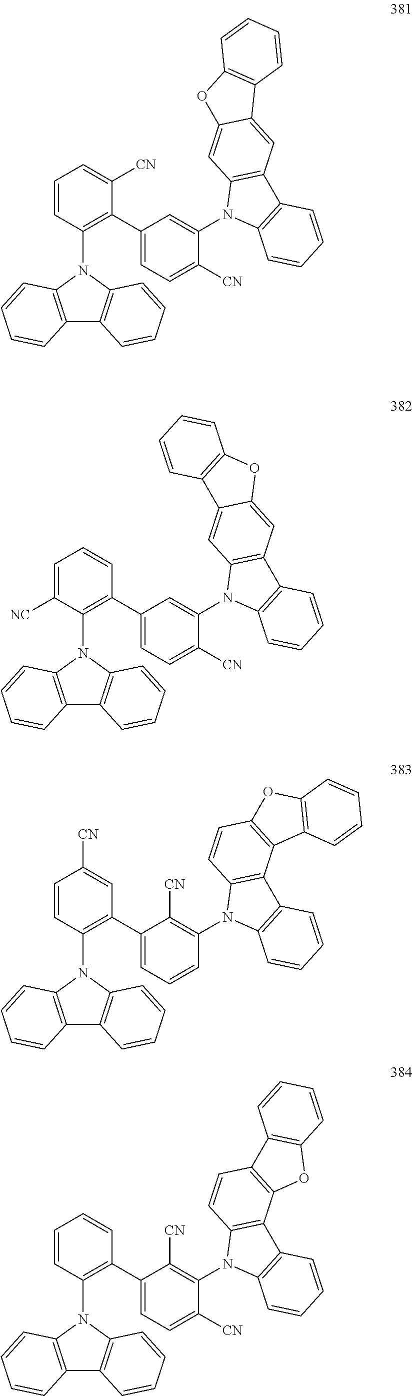 [SMILES: N#Cc1ccc(-c2c(C#N)cccc2-n2c3ccccc3c3ccccc32)cc1-n1c2ccccc2c2cc3c(cc21)oc1ccccc13 ; N#Cc1ccc(-c2cccc(C#N)c2-n2c3ccccc3c3ccccc32)cc1-n1c2ccccc2c2cc3oc4ccccc4c3cc21 ; N#Cc1ccc(-n2c3ccccc3c3ccccc32)c(-c2cccc(-n3c4ccccc4c4c5c(ccc43)oc3ccccc35)c2C#N)c1 ; N#Cc1ccc(-c2ccccc2-n2c3ccccc3c3ccccc32)c(C#N)c1-n1c2ccccc2c2c3oc4ccccc4c3ccc21]

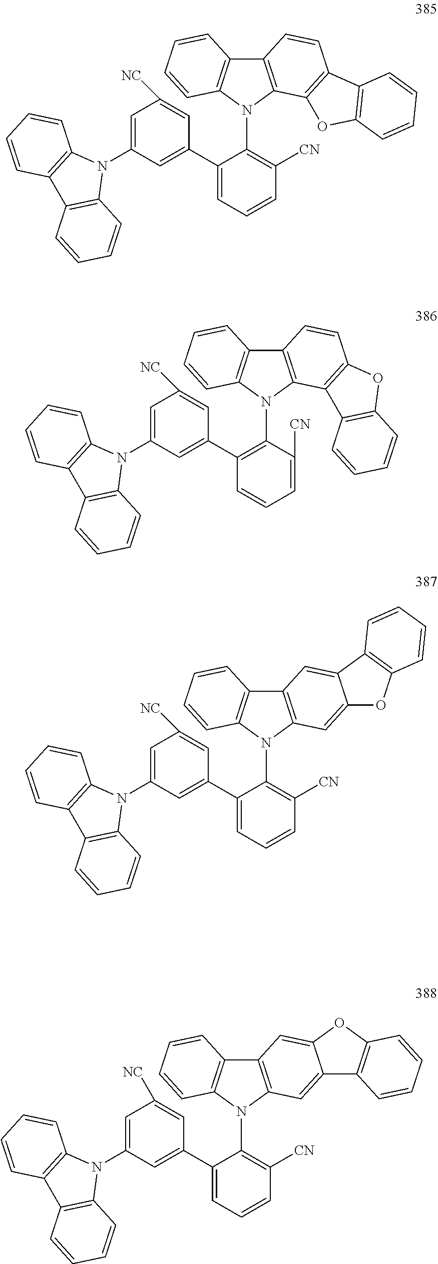 [SMILES: N#Cc1cc(-c2cccc(C#N)c2-n2c3ccccc3c3ccc4c5ccccc5oc4c32)cc(-n2c3ccccc3c3ccccc32)c1 ; N#Cc1cc(-c2cccc(C#N)c2-n2c3ccccc3c3ccc4oc5ccccc5c4c32)cc(-n2c3ccccc3c3ccccc32)c1 ; N#Cc1cc(-c2cccc(C#N)c2-n2c3ccccc3c3cc4c(cc32)oc2ccccc24)cc(-n2c3ccccc3c3ccccc32)c1 ; N#Cc1cc(-c2cccc(C#N)c2-n2c3ccccc3c3cc4oc5ccccc5c4cc32)cc(-n2c3ccccc3c3ccccc32)c1]

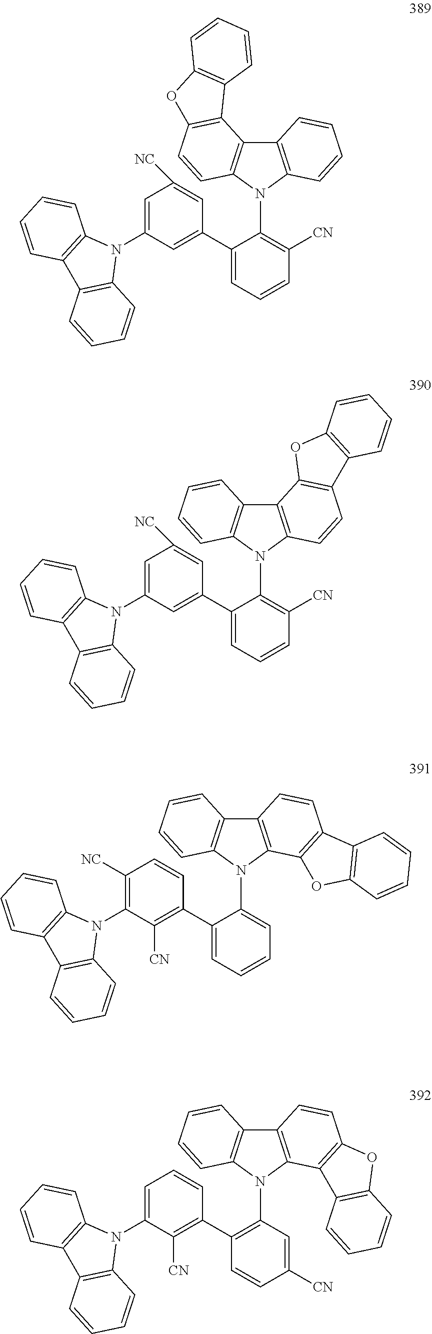 [SMILES: N#Cc1cc(-c2cccc(C#N)c2-n2c3ccccc3c3c4c(ccc32)oc2ccccc24)cc(-n2c3ccccc3c3ccccc32)c1 ; N#Cc1cc(-c2cccc(C#N)c2-n2c3ccccc3c3c4oc5ccccc5c4ccc32)cc(-n2c3ccccc3c3ccccc32)c1 ; N#Cc1ccc(-c2ccccc2-n2c3ccccc3c3ccc4c5ccccc5oc4c32)c(C#N)c1-n1c2ccccc2c2ccccc21 ; N#Cc1ccc(-c2cccc(-n3c4ccccc4c4ccccc43)c2C#N)c(-n2c3ccccc3c3ccc4oc5ccccc5c4c32)c1]

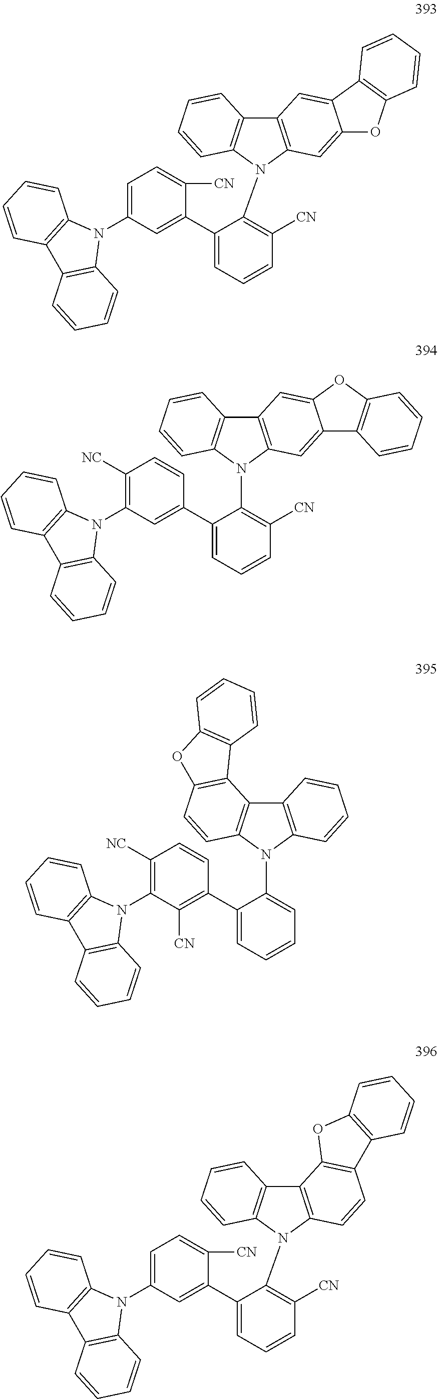 [SMILES: N#Cc1ccc(-n2c3ccccc3c3ccccc32)cc1-c1cccc(C#N)c1-n1c2ccccc2c2cc3c(cc21)oc1ccccc13 ; N#Cc1ccc(-c2cccc(C#N)c2-n2c3ccccc3c3cc4oc5ccccc5c4cc32)cc1-n1c2ccccc2c2ccccc21 ; N#Cc1ccc(-c2ccccc2-n2c3ccccc3c3c4c(ccc32)oc2ccccc24)c(C#N)c1-n1c2ccccc2c2ccccc21 ; N#Cc1ccc(-n2c3ccccc3c3ccccc32)cc1-c1cccc(C#N)c1-n1c2ccccc2c2c3oc4ccccc4c3ccc21]

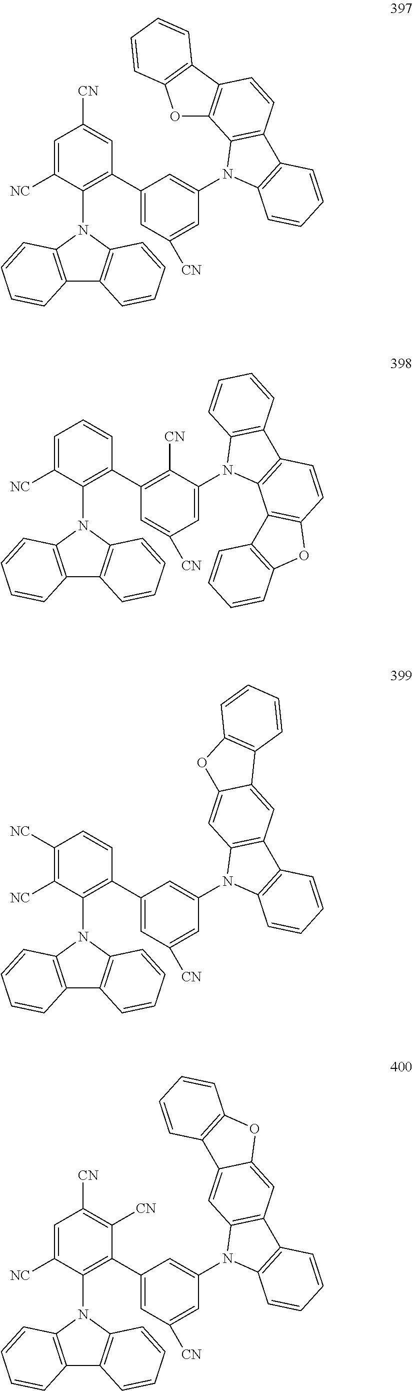 [SMILES: N#Cc1cc(-c2cc(C#N)cc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)c(C#N)c(-n2c3ccccc3c3ccc4oc5ccccc5c4c32)c1 ; N#Cc1cc(-c2ccc(C#N)c(C#N)c2-n2c3ccccc3c3ccccc32)cc(-n2c3ccccc3c3cc4c(cc32)oc2ccccc24)c1 ; N#Cc1cc(-c2c(C#N)c(C#N)cc(C#N)c2-n2c3ccccc3c3ccccc32)cc(-n2c3ccccc3c3cc4oc5ccccc5c4cc32)c1]

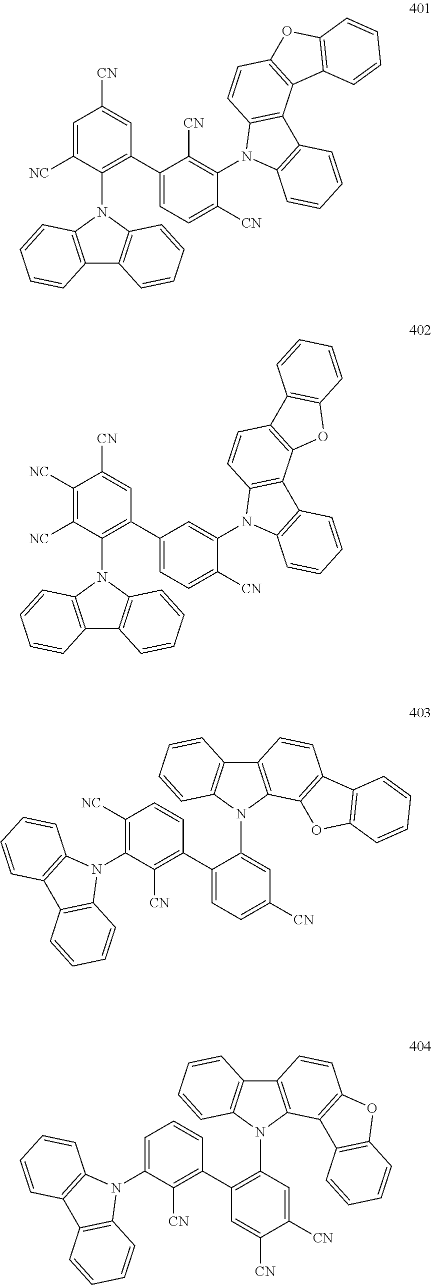 [SMILES: N#Cc1cc(C#N)c(-n2c3ccccc3c3ccccc32)c(-c2ccc(C#N)c(-n3c4ccccc4c4c5c(ccc43)oc3ccccc35)c2C#N)c1 ; N#Cc1ccc(-c2cc(C#N)c(C#N)c(C#N)c2-n2c3ccccc3c3ccccc32)cc1-n1c2ccccc2c2c3oc4ccccc4c3ccc21 ; N#Cc1ccc(-c2ccc(C#N)c(-n3c4ccccc4c4ccccc43)c2C#N)c(-n2c3ccccc3c3ccc4c5ccccc5oc4c32)c1 ; N#Cc1cc(-c2cccc(-n3c4ccccc4c4ccccc43)c2C#N)c(-n2c3ccccc3c3ccc4oc5ccccc5c4c32)cc1C#N]

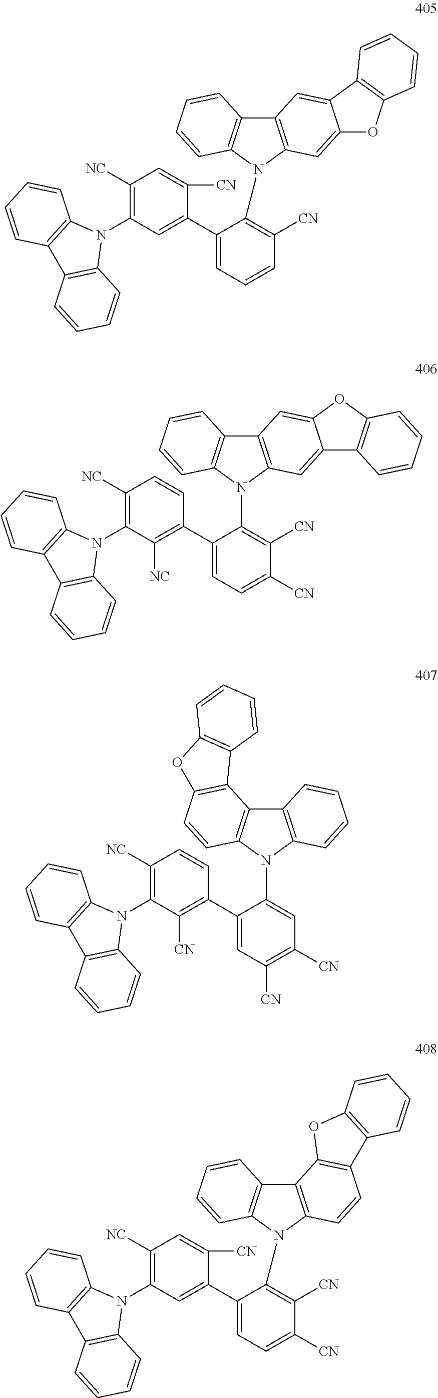

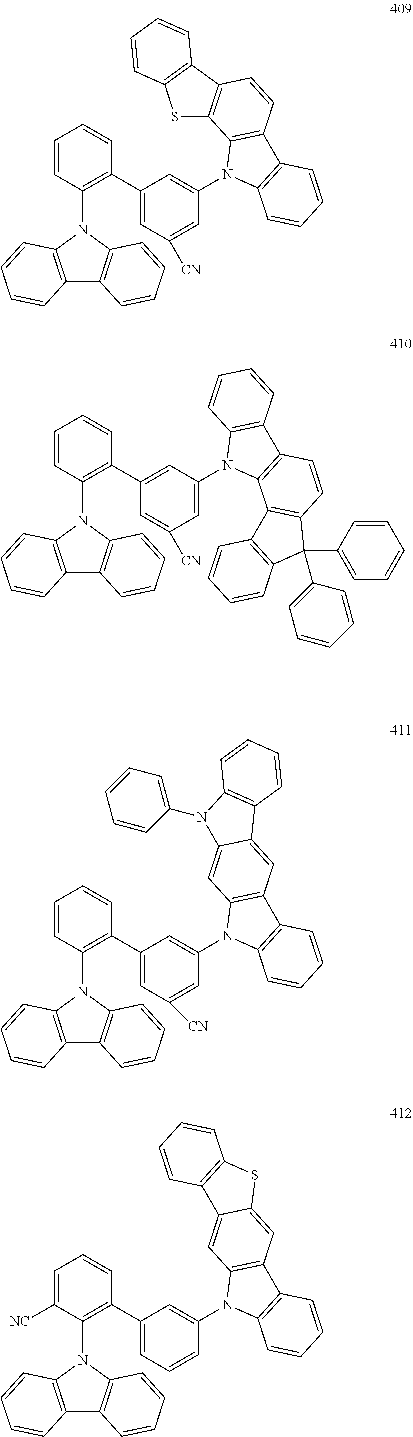 [SMILES: N#Cc1cc(-c2ccccc2-n2c3ccccc3c3ccccc32)cc(-n2c3ccccc3c3ccc4c5ccccc5sc4c32)c1 ; N#Cc1cc(-c2ccccc2-n2c3ccccc3c3ccccc32)cc(-n2c3ccccc3c3ccc4c(c32)-c2ccccc2C4(c2ccccc2)c2ccccc2)c1 ; N#Cc1cc(-c2ccccc2-n2c3ccccc3c3ccccc32)cc(-n2c3ccccc3c3cc4c5ccccc5n(-c5ccccc5)c4cc32)c1 ; N#Cc1cccc(-c2cccc(-n3c4ccccc4c4cc5sc6ccccc6c5cc43)c2)c1-n1c2ccccc2c2ccccc21]

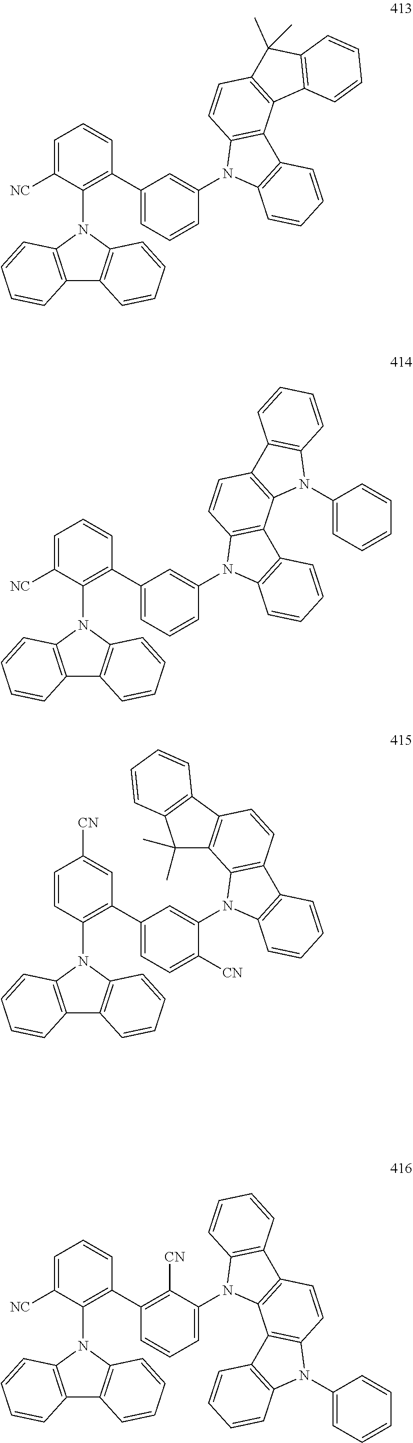 [SMILES: CC1(C)c2ccccc2-c2c1ccc1c2c2ccccc2n1-c1cccc(-c2cccc(C#N)c2-n2c3ccccc3c3ccccc32)c1 ; N#Cc1cccc(-c2cccc(-n3c4ccccc4c4c3ccc3c5ccccc5n(-c5ccccc5)c34)c2)c1-n1c2ccccc2c2ccccc21 ; CC1(C)c2ccccc2-c2ccc3c4ccccc4n(-c4cc(-c5cc(C#N)ccc5-n5c6ccccc6c6ccccc65)ccc4C#N)c3c21 ; N#Cc1cccc(-c2cccc(-n3c4ccccc4c4ccc5c(c6ccccc6n5-c5ccccc5)c43)c2C#N)c1-n1c2ccccc2c2ccccc21]

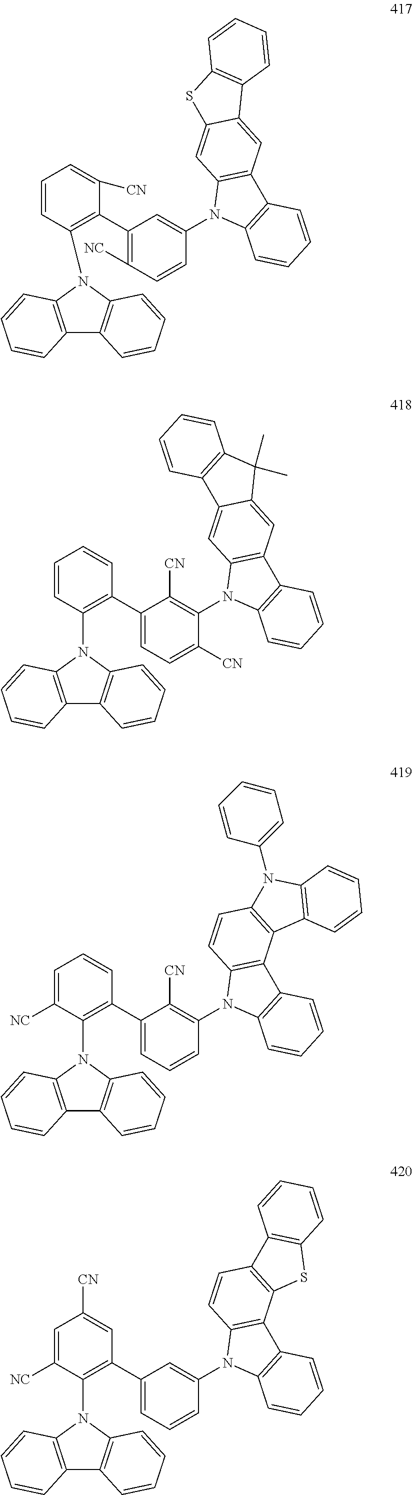 [SMILES: N#Cc1ccc(-n2c3ccccc3c3cc4c(cc32)sc2ccccc24)cc1-c1c(C#N)cccc1-n1c2ccccc2c2ccccc21 ; CC1(C)c2ccccc2-c2cc3c(cc21)c1ccccc1n3-c1c(C#N)ccc(-c2ccccc2-n2c3ccccc3c3ccccc32)c1C#N ; N#Cc1cccc(-c2cccc(-n3c4ccccc4c4c5c6ccccc6n(-c6ccccc6)c5ccc43)c2C#N)c1-n1c2ccccc2c2ccccc21 ; N#Cc1cc(C#N)c(-n2c3ccccc3c3ccccc32)c(-c2cccc(-n3c4ccccc4c4c5sc6ccccc6c5ccc43)c2)c1]

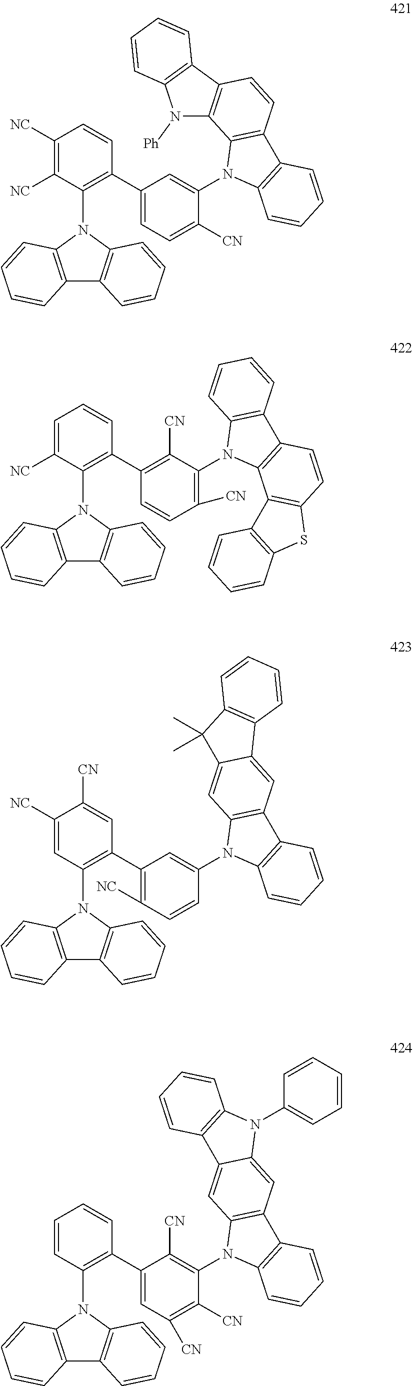 [SMILES: N#Cc1ccc(-c2ccc(C#N)c(C#N)c2-n2c3ccccc3c3ccccc32)cc1-n1c2ccccc2c2ccc3c4ccccc4n(-c4ccccc4)c3c21 ; N#Cc1cccc(-c2ccc(C#N)c(-n3c4ccccc4c4ccc5sc6ccccc6c5c43)c2C#N)c1-n1c2ccccc2c2ccccc21 ; CC1(C)c2ccccc2-c2cc3c4ccccc4n(-c4ccc(C#N)c(-c5cc(C#N)c(C#N)cc5-n5c6ccccc6c6ccccc65)c4)c3cc21 ; N#Cc1cc(-c2ccccc2-n2c3ccccc3c3ccccc32)c(C#N)c(-n2c3ccccc3c3cc4c(cc32)c2ccccc2n4-c2ccccc2)c1C#N]

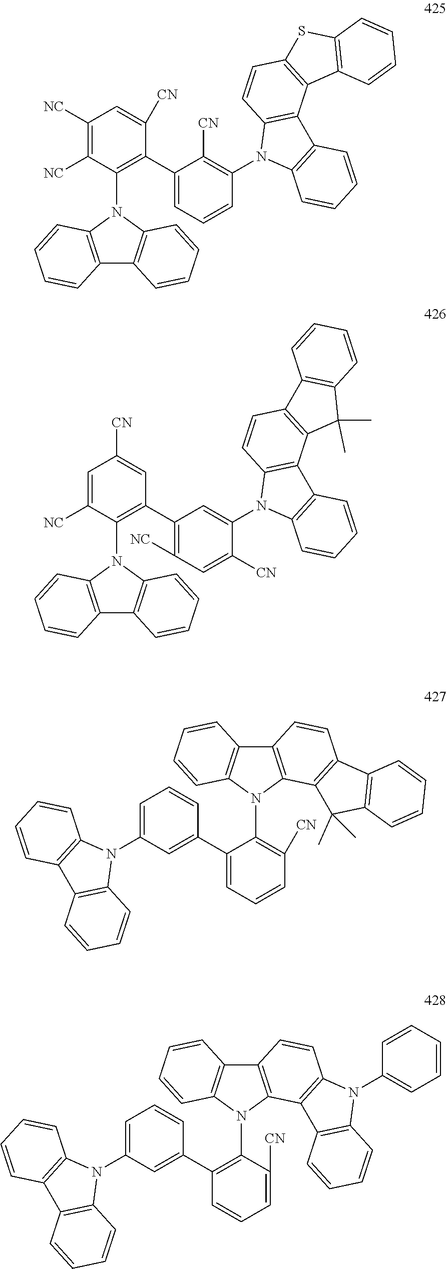 [SMILES: N#Cc1cc(C#N)c(-c2cccc(-n3c4ccccc4c4c5c(ccc43)sc3ccccc35)c2C#N)c(-n2c3ccccc3c3ccccc32)c1C#N ; CC1(C)c2ccccc2-c2ccc3c(c21)c1ccccc1n3-c1cc(-c2cc(C#N)cc(C#N)c2-n2c3ccccc3c3ccccc32)c(C#N)cc1C#N ; CC1(C)c2ccccc2-c2ccc3c4ccccc4n(-c4c(C#N)cccc4-c4cccc(-n5c6ccccc6c6ccccc65)c4)c3c21 ; N#Cc1cccc(-c2cccc(-n3c4ccccc4c4ccccc43)c2)c1-n1c2ccccc2c2ccc3c(c4ccccc4n3-c3ccccc3)c21]

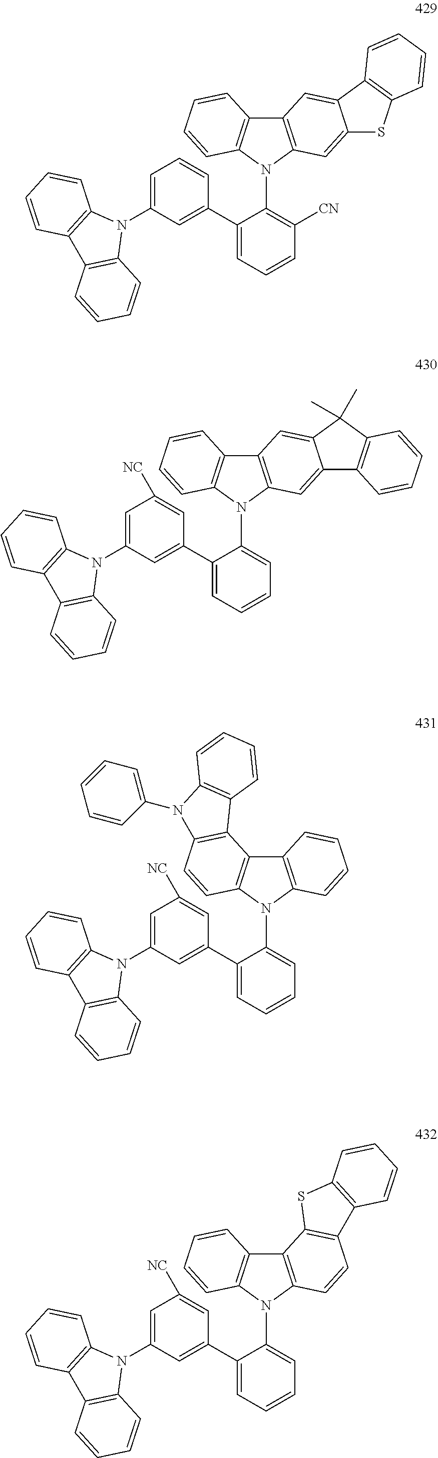 [SMILES: N#Cc1cccc(-c2cccc(-n3c4ccccc4c4ccccc43)c2)c1-n1c2ccccc2c2cc3c(cc21)sc1ccccc13 ; CC1(C)c2ccccc2-c2cc3c(cc21)c1ccccc1n3-c1ccccc1-c1cc(C#N)cc(-n2c3ccccc3c3ccccc32)c1 ; N#Cc1cc(-c2ccccc2-n2c3ccccc3c3c4c5ccccc5n(-c5ccccc5)c4ccc32)cc(-n2c3ccccc3c3ccccc32)c1 ; N#Cc1cc(-c2ccccc2-n2c3ccccc3c3c4sc5ccccc5c4ccc32)cc(-n2c3ccccc3c3ccccc32)c1]

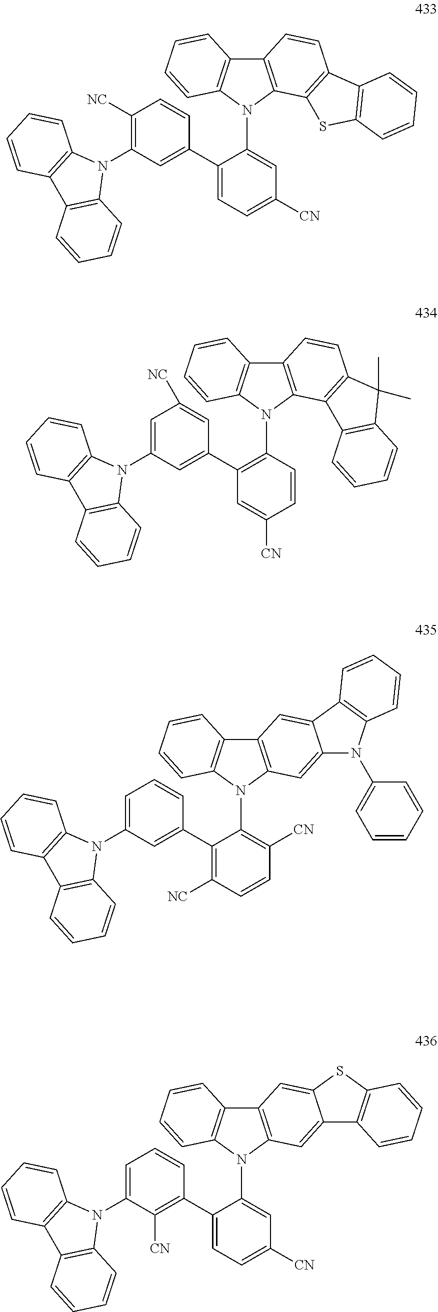 [SMILES: N#Cc1ccc(-c2ccc(C#N)c(-n3c4ccccc4c4ccccc43)c2)c(-n2c3ccccc3c3ccc4c5ccccc5sc4c32)c1 ; CC1(C)c2ccccc2-c2c1ccc1c3ccccc3n(-c3ccc(C#N)cc3-c3cc(C#N)cc(-n4c5ccccc5c5ccccc54)c3)c21 ; N#Cc1ccc(C#N)c(-n2c3ccccc3c3cc4c5ccccc5n(-c5ccccc5)c4cc32)c1-c1cccc(-n2c3ccccc3c3ccccc32)c1 ; N#Cc1ccc(-c2cccc(-n3c4ccccc4c4ccccc43)c2C#N)c(-n2c3ccccc3c3cc4sc5ccccc5c4cc32)c1]

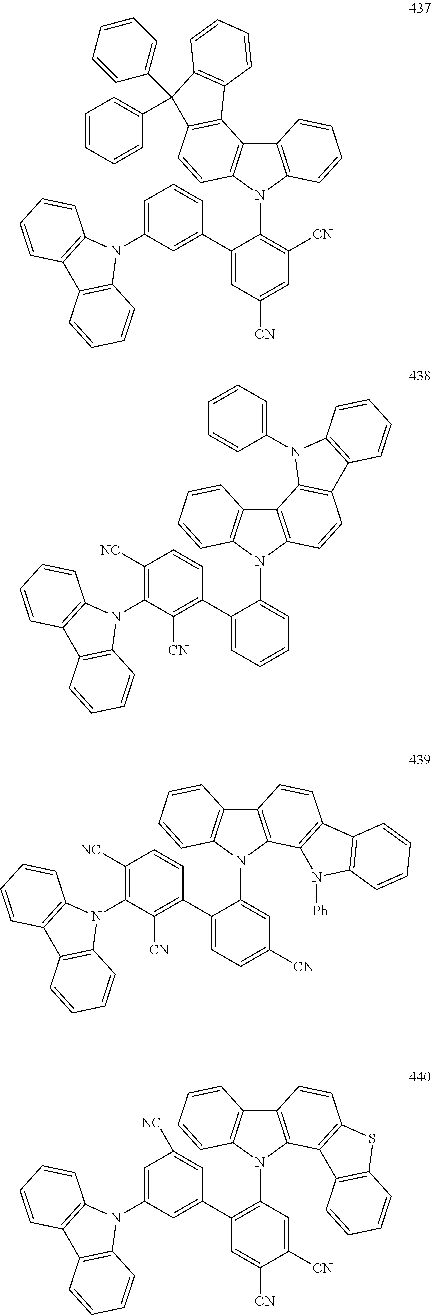 [SMILES: N#Cc1cc(C#N)c(-n2c3ccccc3c3c4c(ccc32)C(c2ccccc2)(c2ccccc2)c2ccccc2-4)c(-c2cccc(-n3c4ccccc4c4ccccc43)c2)c1 ; N#Cc1ccc(-c2ccccc2-n2c3ccccc3c3c2ccc2c4ccccc4n(-c4ccccc4)c23)c(C#N)c1-n1c2ccccc2c2ccccc21 ; N#Cc1ccc(-c2ccc(C#N)c(-n3c4ccccc4c4ccccc43)c2C#N)c(-n2c3ccccc3c3ccc4c5ccccc5n(-c5ccccc5)c4c32)c1 ; N#Cc1cc(-c2cc(C#N)c(C#N)cc2-n2c3ccccc3c3ccc4sc5ccccc5c4c32)cc(-n2c3ccccc3c3ccccc32)c1]

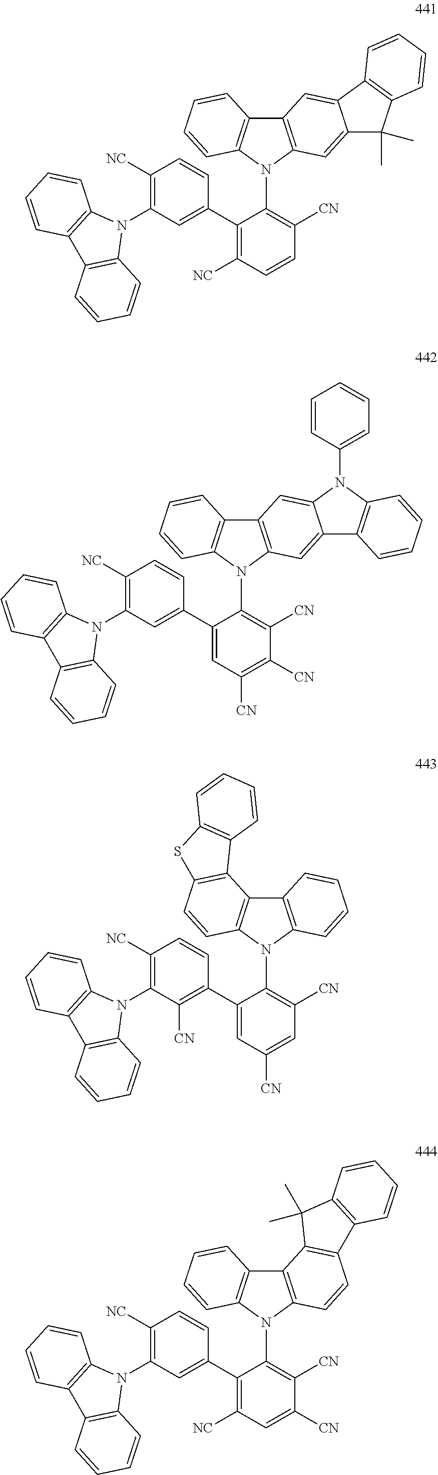 [SMILES: CC1(C)c2ccccc2-c2cc3c4ccccc4n(-c4c(C#N)ccc(C#N)c4-c4ccc(C#N)c(-n5c6ccccc6c6ccccc65)c4)c3cc21 ; N#Cc1ccc(-c2cc(C#N)c(C#N)c(C#N)c2-n2c3ccccc3c3cc4c(cc32)c2ccccc2n4-c2ccccc2)cc1-n1c2ccccc2c2ccccc21 ; N#Cc1cc(C#N)c(-n2c3ccccc3c3c4c(ccc32)sc2ccccc24)c(-c2ccc(C#N)c(-n3c4ccccc4c4ccccc43)c2C#N)c1 ; CC1(C)c2ccccc2-c2ccc3c(c21)c1ccccc1n3-c1c(C#N)c(C#N)cc(C#N)c1-c1ccc(C#N)c(-n2c3ccccc3c3ccccc32)c1]

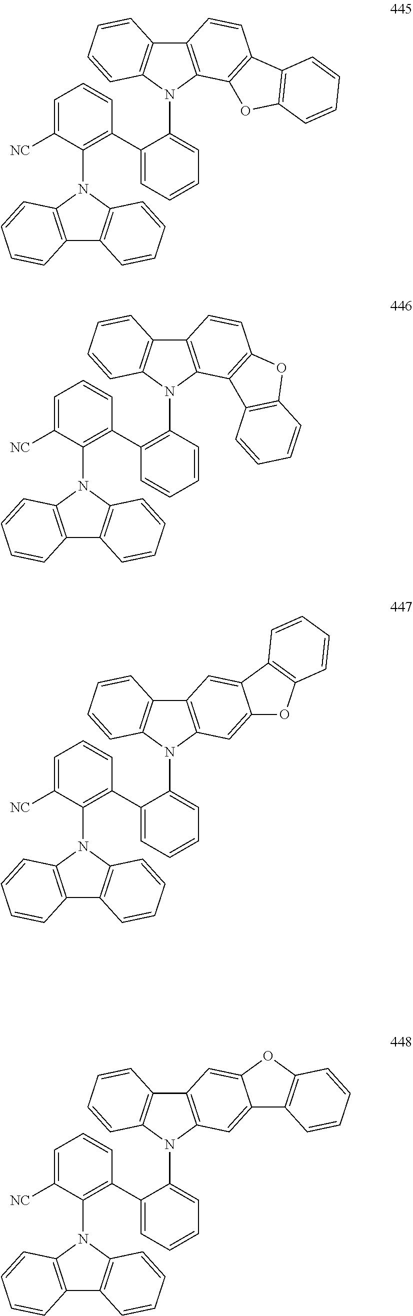 [SMILES: N#Cc1cccc(-c2ccccc2-n2c3ccccc3c3ccc4c5ccccc5oc4c32)c1-n1c2ccccc2c2ccccc21 ; N#Cc1cccc(-c2ccccc2-n2c3ccccc3c3ccc4oc5ccccc5c4c32)c1-n1c2ccccc2c2ccccc21 ; N#Cc1cccc(-c2ccccc2-n2c3ccccc3c3cc4c(cc32)oc2ccccc24)c1-n1c2ccccc2c2ccccc21 ; N#Cc1cccc(-c2ccccc2-n2c3ccccc3c3cc4oc5ccccc5c4cc32)c1-n1c2ccccc2c2ccccc21]

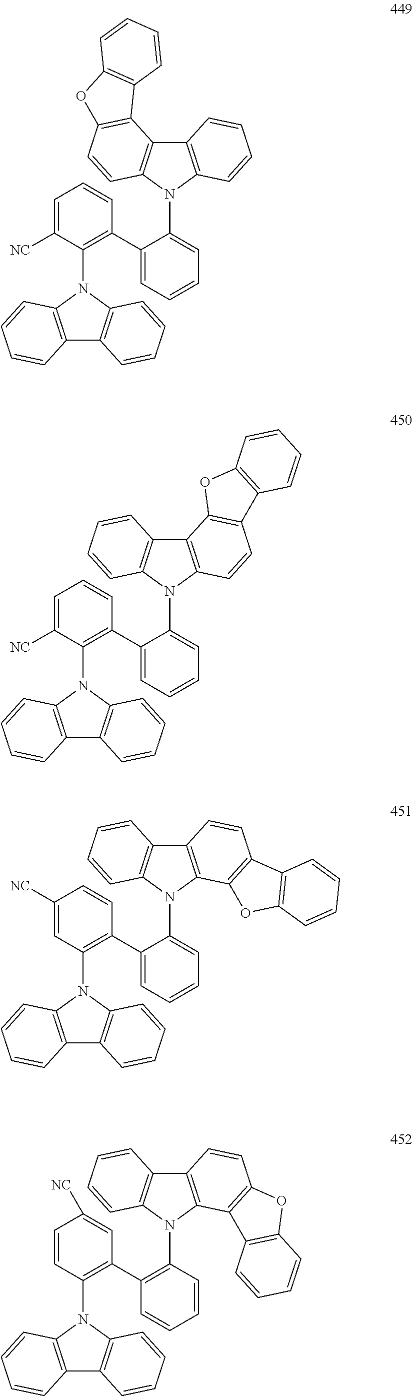 [SMILES: N#Cc1cccc(-c2ccccc2-n2c3ccccc3c3c4c(ccc32)oc2ccccc24)c1-n1c2ccccc2c2ccccc21 ; N#Cc1cccc(-c2ccccc2-n2c3ccccc3c3c4oc5ccccc5c4ccc32)c1-n1c2ccccc2c2ccccc21 ; N#Cc1ccc(-c2ccccc2-n2c3ccccc3c3ccc4c5ccccc5oc4c32)c(-n2c3ccccc3c3ccccc32)c1 ; N#Cc1ccc(-n2c3ccccc3c3ccccc32)c(-c2ccccc2-n2c3ccccc3c3ccc4oc5ccccc5c4c32)c1]

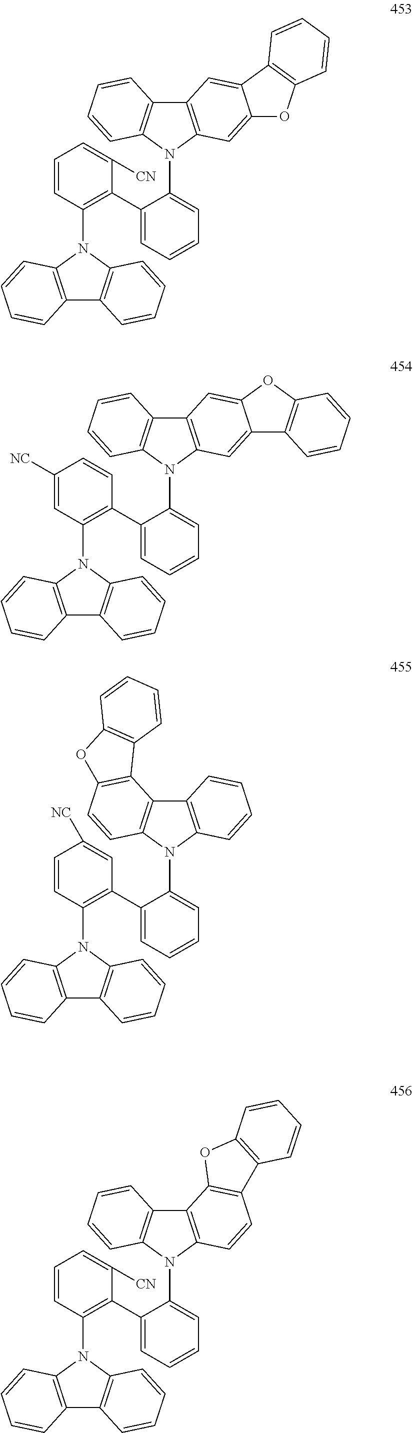

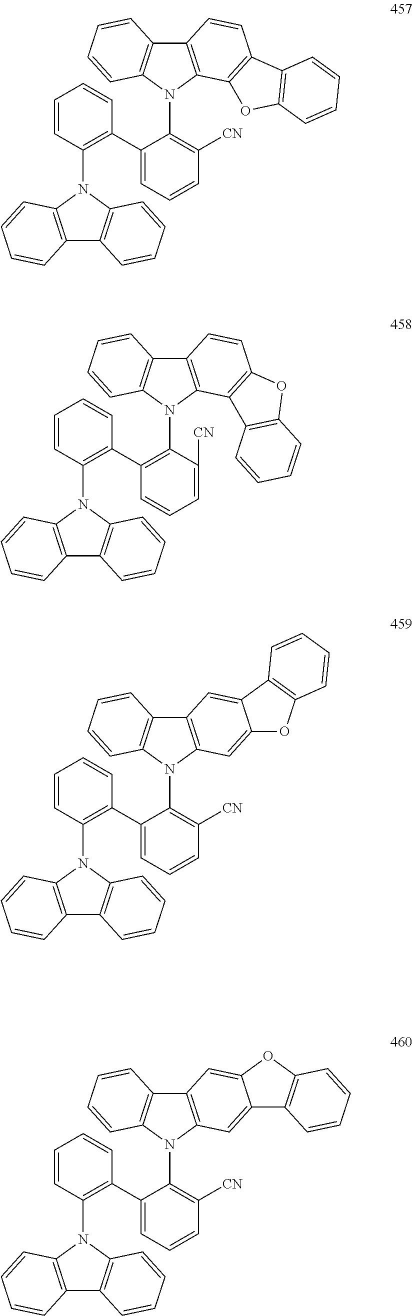 [SMILES: N#Cc1cccc(-c2ccccc2-n2c3ccccc3c3ccccc32)c1-n1c2ccccc2c2ccc3c4ccccc4oc3c21 ; N#Cc1cccc(-c2ccccc2-n2c3ccccc3c3ccccc32)c1-n1c2ccccc2c2ccc3oc4ccccc4c3c21 ; N#Cc1cccc(-c2ccccc2-n2c3ccccc3c3ccccc32)c1-n1c2ccccc2c2cc3c(cc21)oc1ccccc13 ; N#Cc1cccc(-c2ccccc2-n2c3ccccc3c3ccccc32)c1-n1c2ccccc2c2cc3oc4ccccc4c3cc21]

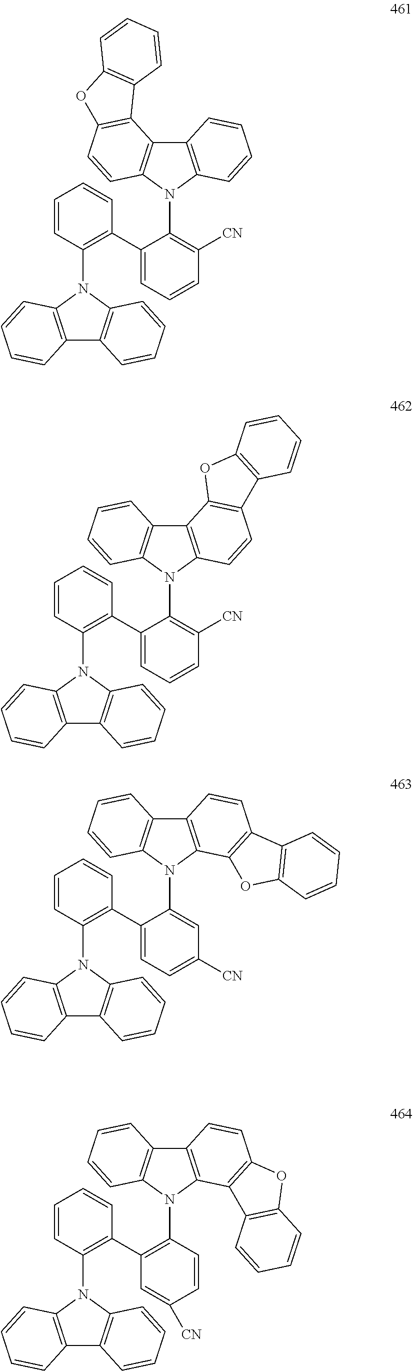 [SMILES: N#Cc1cccc(-c2ccccc2-n2c3ccccc3c3ccccc32)c1-n1c2ccccc2c2c3c(ccc21)oc1ccccc13 ; N#Cc1cccc(-c2ccccc2-n2c3ccccc3c3ccccc32)c1-n1c2ccccc2c2c3oc4ccccc4c3ccc21 ; N#Cc1ccc(-c2ccccc2-n2c3ccccc3c3ccccc32)c(-n2c3ccccc3c3ccc4c5ccccc5oc4c32)c1 ; N#Cc1ccc(-n2c3ccccc3c3ccc4oc5ccccc5c4c32)c(-c2ccccc2-n2c3ccccc3c3ccccc32)c1]

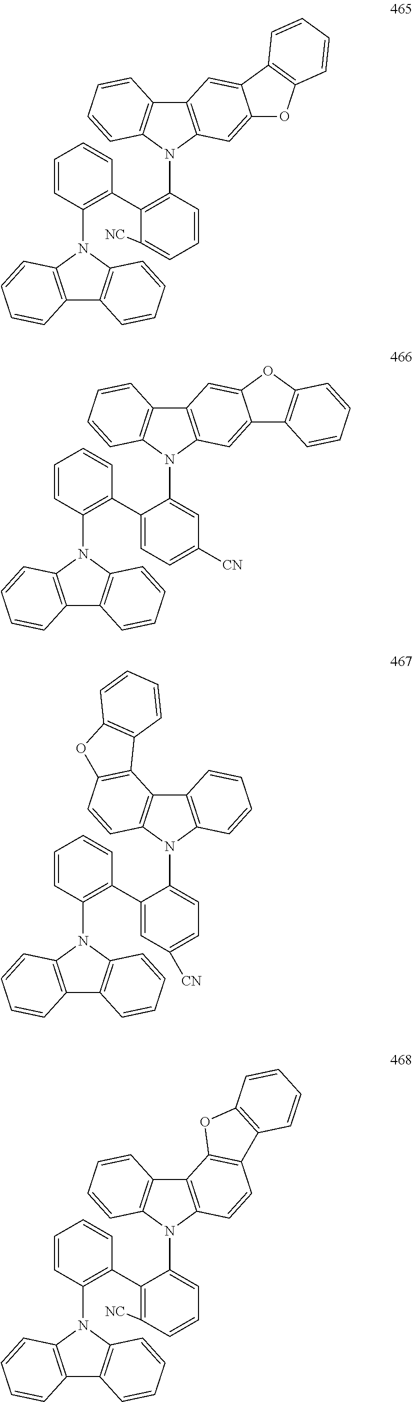

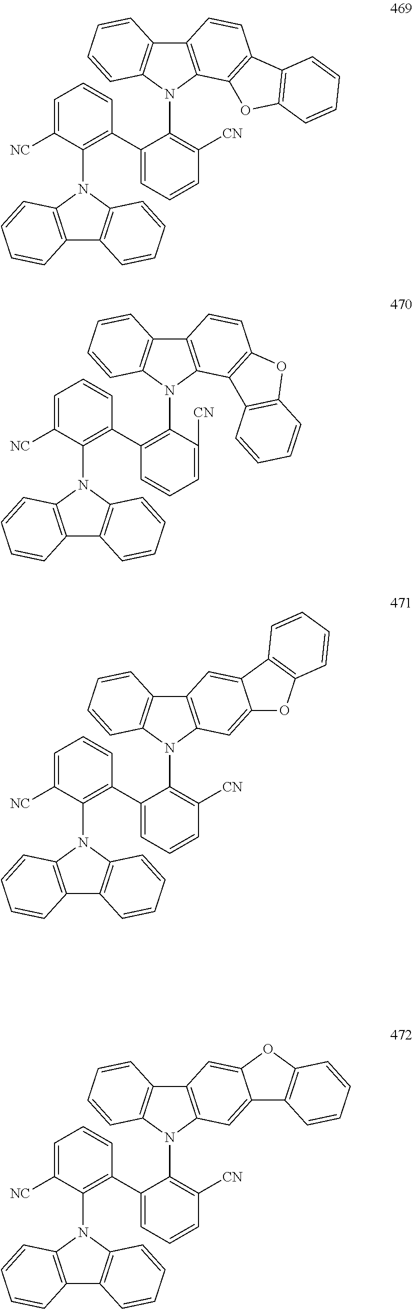 [SMILES: N#Cc1cccc(-c2cccc(C#N)c2-n2c3ccccc3c3ccc4c5ccccc5oc4c32)c1-n1c2ccccc2c2ccccc21 ; N#Cc1cccc(-c2cccc(C#N)c2-n2c3ccccc3c3ccc4oc5ccccc5c4c32)c1-n1c2ccccc2c2ccccc21 ; N#Cc1cccc(-c2cccc(C#N)c2-n2c3ccccc3c3cc4c(cc32)oc2ccccc24)c1-n1c2ccccc2c2ccccc21 ; N#Cc1cccc(-c2cccc(C#N)c2-n2c3ccccc3c3cc4oc5ccccc5c4cc32)c1-n1c2ccccc2c2ccccc21]

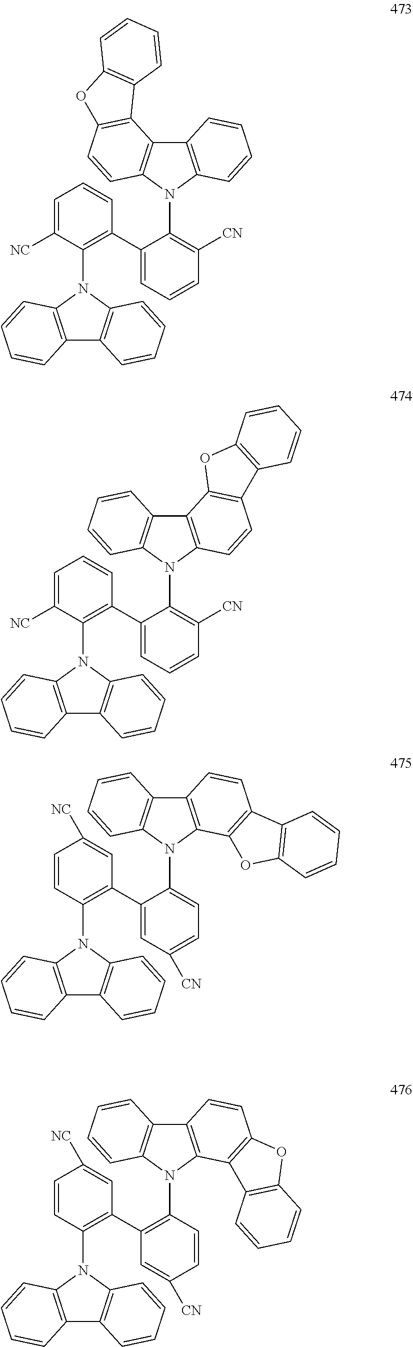

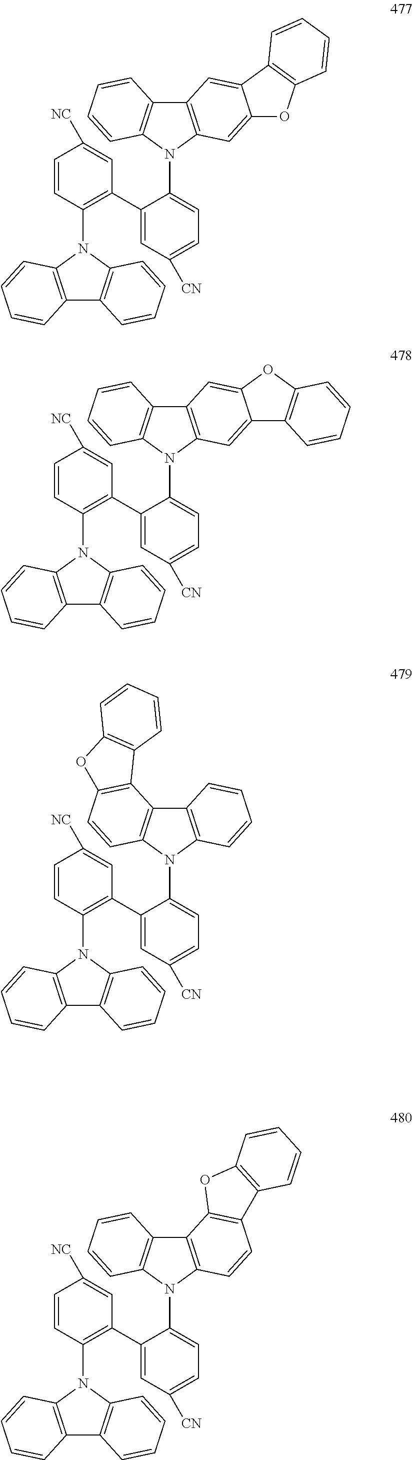 [SMILES: N#Cc1ccc(-n2c3ccccc3c3ccccc32)c(-c2cc(C#N)ccc2-n2c3ccccc3c3cc4c(cc32)oc2ccccc24)c1 ; N#Cc1ccc(-n2c3ccccc3c3ccccc32)c(-c2cc(C#N)ccc2-n2c3ccccc3c3cc4oc5ccccc5c4cc32)c1 ; N#Cc1ccc(-n2c3ccccc3c3ccccc32)c(-c2cc(C#N)ccc2-n2c3ccccc3c3c4c(ccc32)oc2ccccc24)c1 ; N#Cc1ccc(-n2c3ccccc3c3ccccc32)c(-c2cc(C#N)ccc2-n2c3ccccc3c3c4oc5ccccc5c4ccc32)c1]

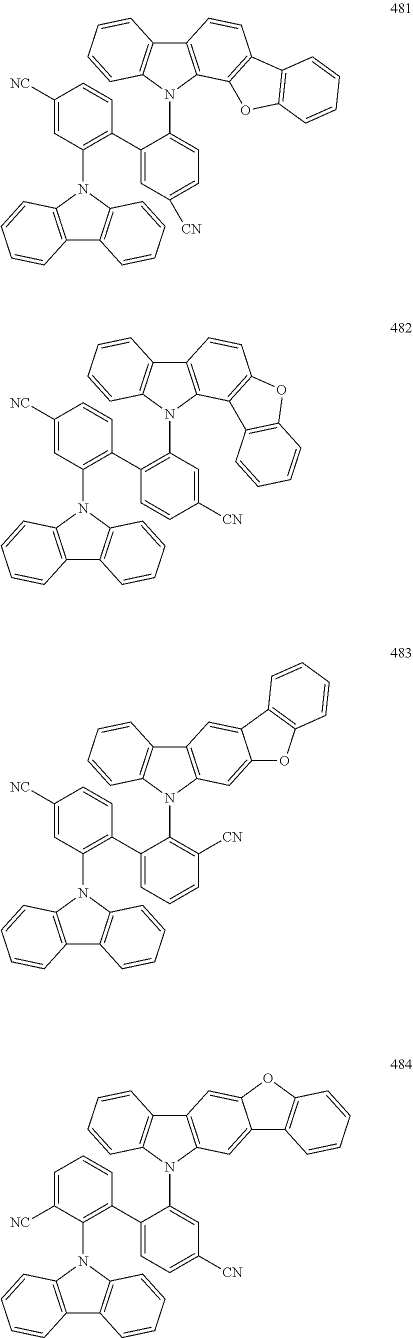 [SMILES: N#Cc1ccc(-n2c3ccccc3c3ccc4c5ccccc5oc4c32)c(-c2ccc(C#N)cc2-n2c3ccccc3c3ccccc32)c1 ; N#Cc1ccc(-c2ccc(C#N)cc2-n2c3ccccc3c3ccc4oc5ccccc5c4c32)c(-n2c3ccccc3c3ccccc32)c1 ; N#Cc1ccc(-c2cccc(C#N)c2-n2c3ccccc3c3cc4c(cc32)oc2ccccc24)c(-n2c3ccccc3c3ccccc32)c1 ; N#Cc1ccc(-c2cccc(C#N)c2-n2c3ccccc3c3ccccc32)c(-n2c3ccccc3c3cc4oc5ccccc5c4cc32)c1]

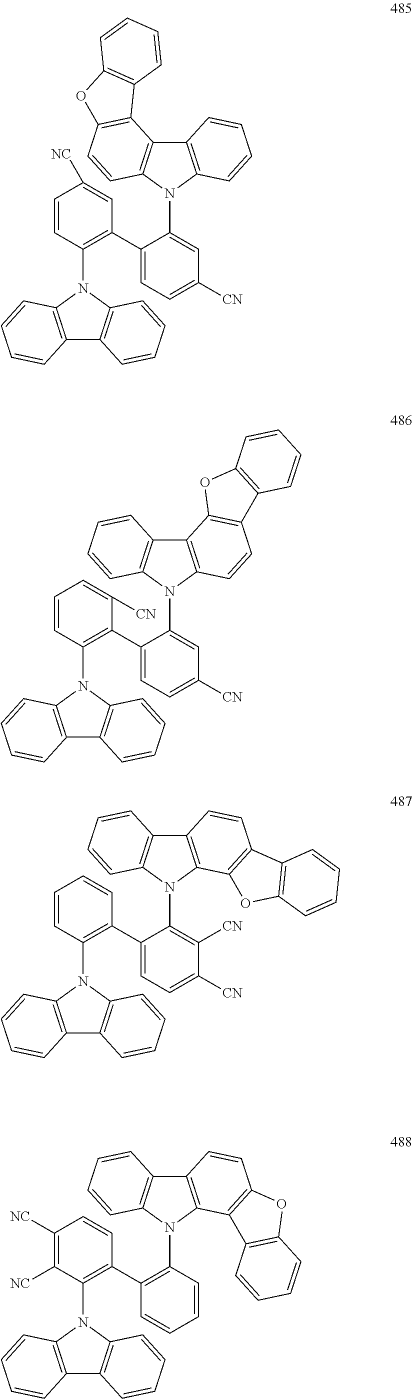 [SMILES: N#Cc1ccc(-n2c3ccccc3c3ccccc32)c(-c2ccc(C#N)cc2-n2c3ccccc3c3c4c(ccc32)oc2ccccc24)c1 ; N#Cc1ccc(-c2c(C#N)cccc2-n2c3ccccc3c3ccccc32)c(-n2c3ccccc3c3c4oc5ccccc5c4ccc32)c1 ; N#Cc1ccc(-c2ccccc2-n2c3ccccc3c3ccccc32)c(-n2c3ccccc3c3ccc4c5ccccc5oc4c32)c1C#N ; N#Cc1ccc(-c2ccccc2-n2c3ccccc3c3ccc4oc5ccccc5c4c32)c(-n2c3ccccc3c3ccccc32)c1C#N]

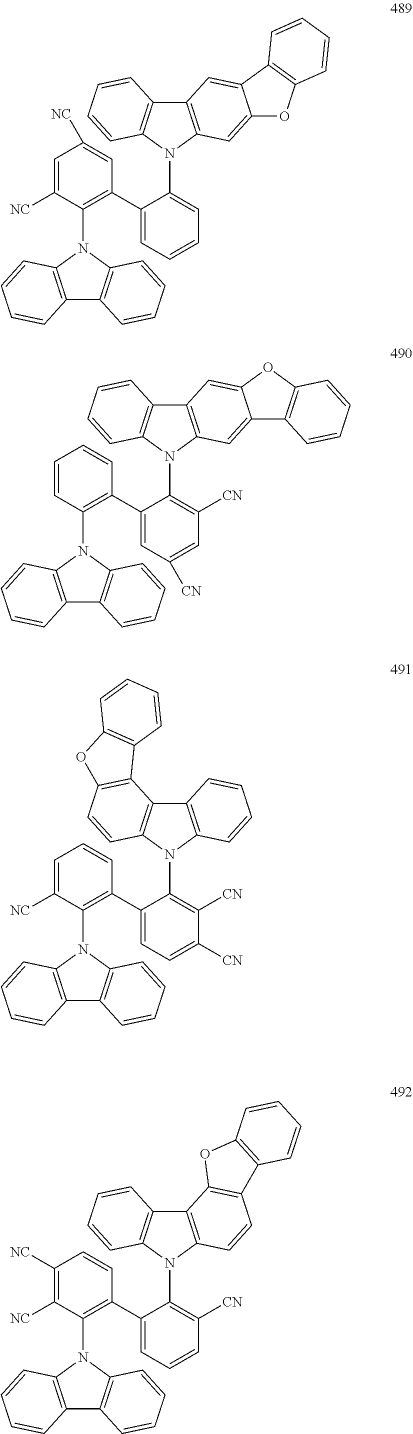 [SMILES: N#Cc1cc(C#N)c(-n2c3ccccc3c3ccccc32)c(-c2ccccc2-n2c3ccccc3c3cc4c(cc32)oc2ccccc24)c1 ; N#Cc1cc(C#N)c(-n2c3ccccc3c3cc4oc5ccccc5c4cc32)c(-c2ccccc2-n2c3ccccc3c3ccccc32)c1 ; N#Cc1ccc(-c2cccc(C#N)c2-n2c3ccccc3c3ccccc32)c(-n2c3ccccc3c3c4c(ccc32)oc2ccccc24)c1C#N ; N#Cc1ccc(-c2cccc(C#N)c2-n2c3ccccc3c3c4oc5ccccc5c4ccc32)c(-n2c3ccccc3c3ccccc32)c1C#N]

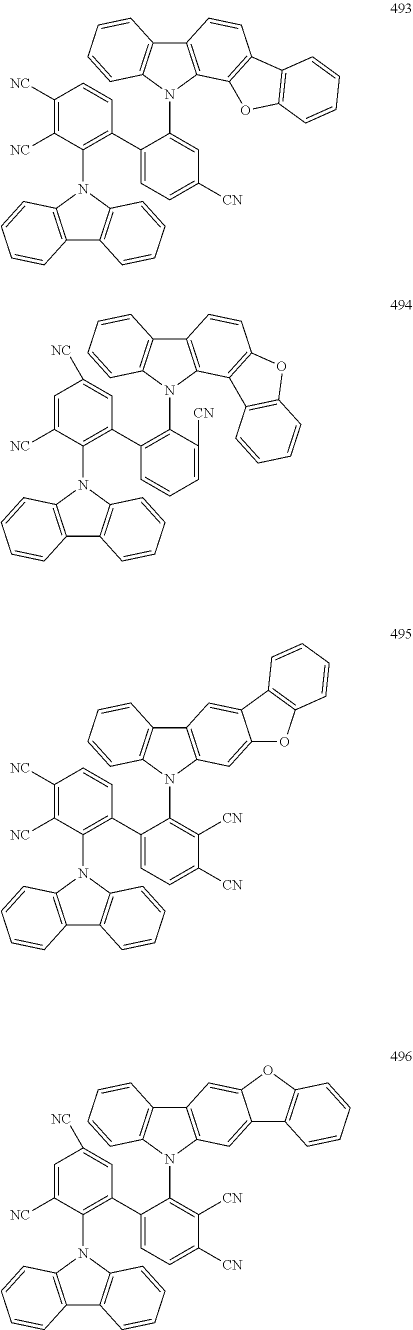 [SMILES: N#Cc1ccc(-c2ccc(C#N)c(C#N)c2-n2c3ccccc3c3ccccc32)c(-n2c3ccccc3c3ccc4c5ccccc5oc4c32)c1 ; N#Cc1cc(C#N)c(-n2c3ccccc3c3ccccc32)c(-c2cccc(C#N)c2-n2c3ccccc3c3ccc4oc5ccccc5c4c32)c1 ; N#Cc1ccc(-c2ccc(C#N)c(C#N)c2-n2c3ccccc3c3cc4c(cc32)oc2ccccc24)c(-n2c3ccccc3c3ccccc32)c1C#N ; N#Cc1cc(C#N)c(-n2c3ccccc3c3ccccc32)c(-c2ccc(C#N)c(C#N)c2-n2c3ccccc3c3cc4oc5ccccc5c4cc32)c1]

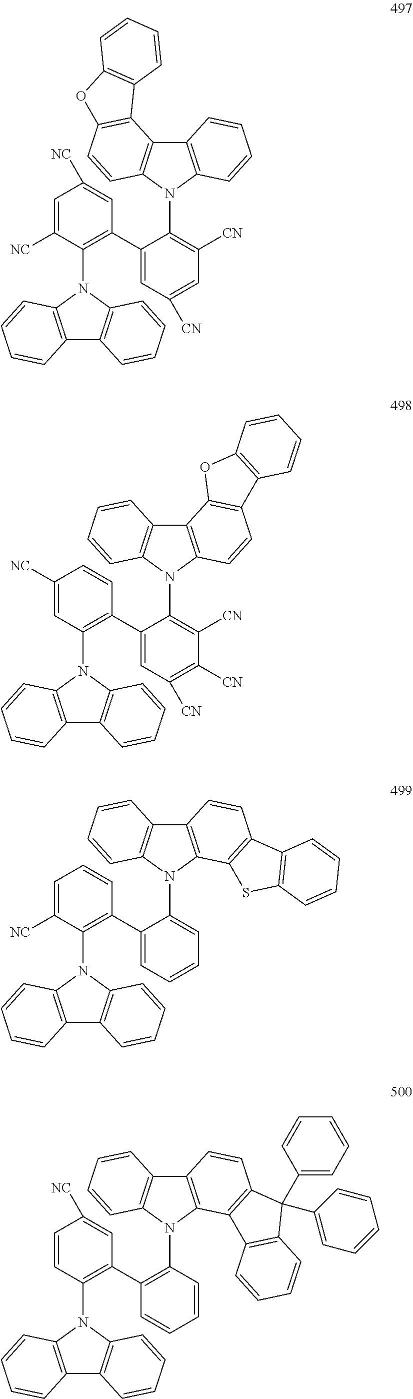 [SMILES: N#Cc1cc(C#N)c(-n2c3ccccc3c3ccccc32)c(-c2cc(C#N)cc(C#N)c2-n2c3ccccc3c3c4c(ccc32)oc2ccccc24)c1 ; N#Cc1ccc(-c2cc(C#N)c(C#N)c(C#N)c2-n2c3ccccc3c3c4oc5ccccc5c4ccc32)c(-n2c3ccccc3c3ccccc32)c1 ; N#Cc1cccc(-c2ccccc2-n2c3ccccc3c3ccc4c5ccccc5sc4c32)c1-n1c2ccccc2c2ccccc21 ; N#Cc1ccc(-n2c3ccccc3c3ccccc32)c(-c2ccccc2-n2c3ccccc3c3ccc4c(c32)-c2ccccc2C4(c2ccccc2)c2ccccc2)c1]

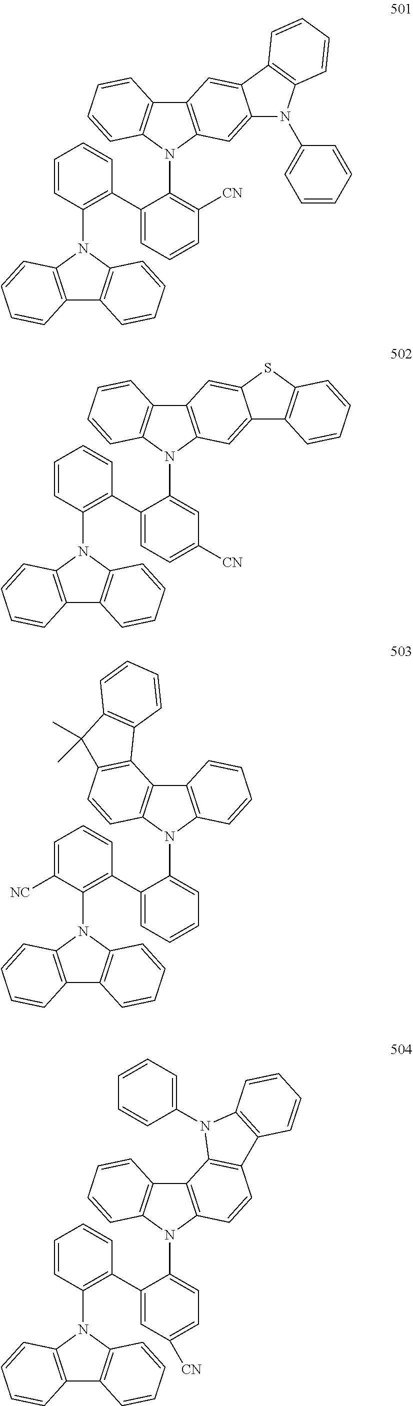

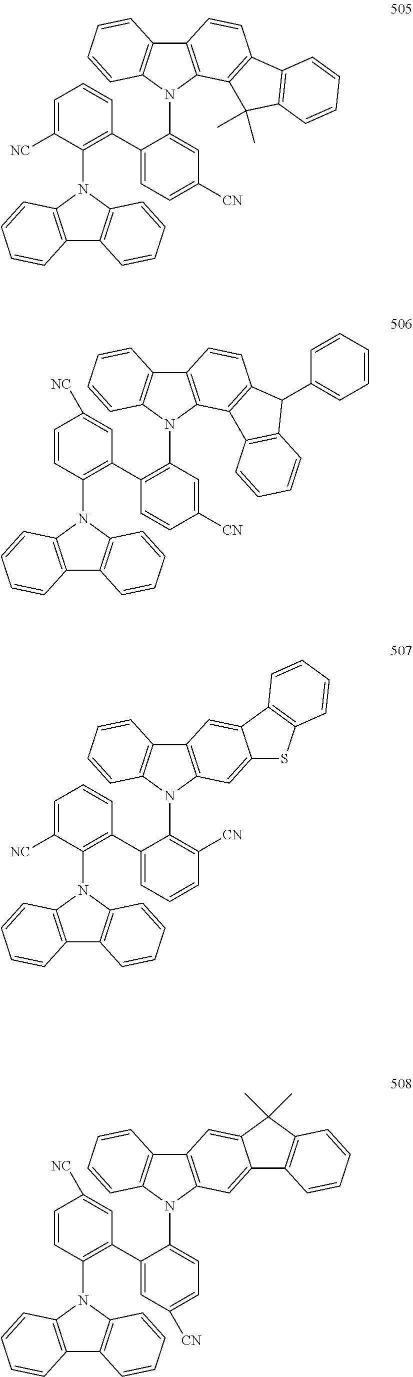 [SMILES: CC1(C)c2ccccc2-c2ccc3c4ccccc4n(-c4cc(C#N)ccc4-c4cccc(C#N)c4-n4c5ccccc5c5ccccc54)c3c21 ; N#Cc1ccc(-n2c3ccccc3c3ccccc32)c(-c2ccc(C#N)cc2-n2c3ccccc3c3ccc4c(c32)-c2ccccc2C4c2ccccc2)c1 ; N#Cc1cccc(-c2cccc(C#N)c2-n2c3ccccc3c3cc4c(cc32)sc2ccccc24)c1-n1c2ccccc2c2ccccc21 ; CC1(C)c2ccccc2-c2cc3c(cc21)c1ccccc1n3-c1ccc(C#N)cc1-c1cc(C#N)ccc1-n1c2ccccc2c2ccccc21]

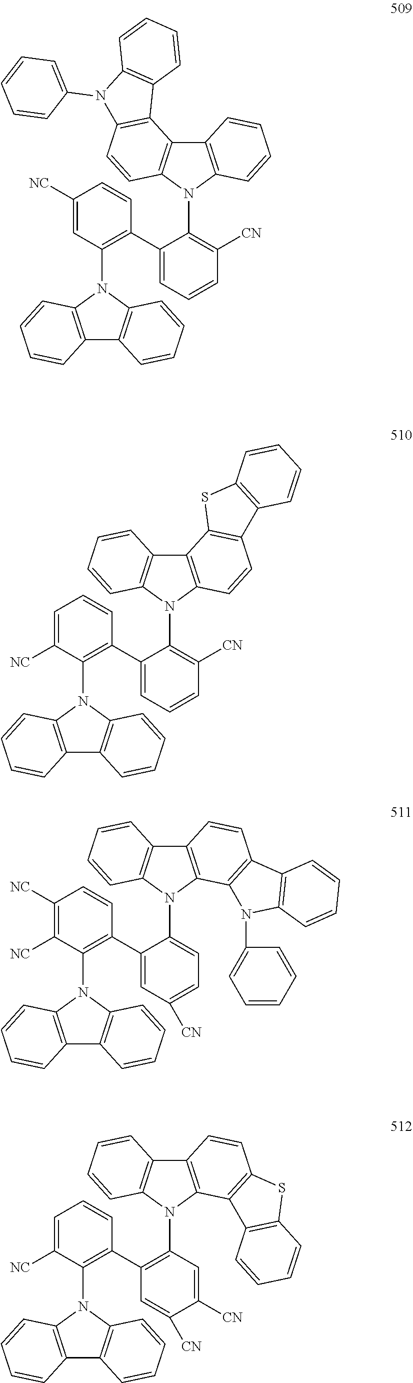

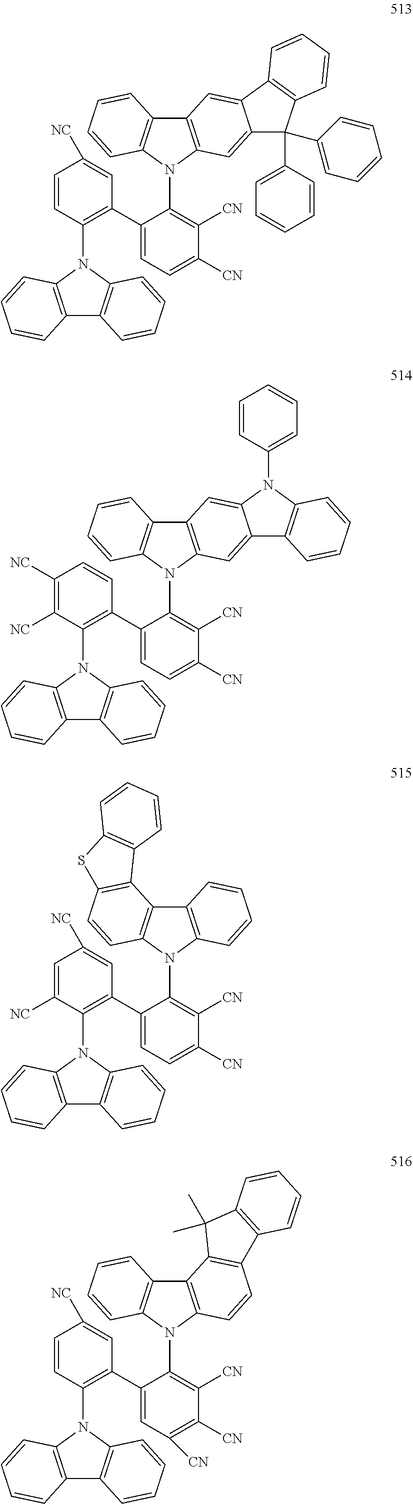

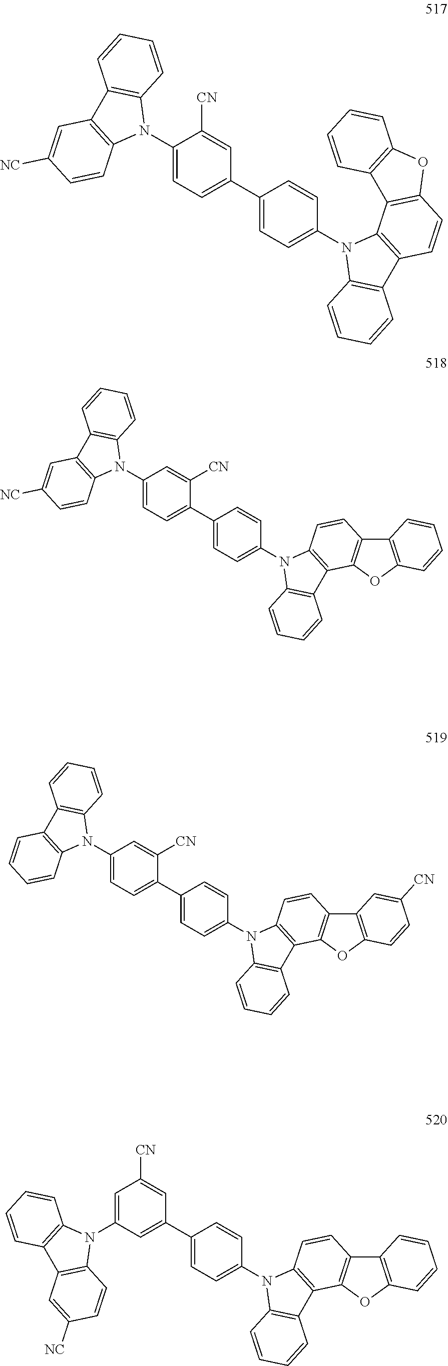 [SMILES: N#Cc1ccc2c(c1)c1ccccc1n2-c1ccc(-c2ccc(-n3c4ccccc4c4ccc5oc6ccccc6c5c43)cc2)cc1C#N ; N#Cc1ccc2c(c1)c1ccccc1n2-c1ccc(-c2ccc(-n3c4ccccc4c4c5oc6ccccc6c5ccc43)cc2)c(C#N)c1 ; N#Cc1ccc2oc3c(ccc4c3c3ccccc3n4-c3ccc(-c4ccc(-n5c6ccccc6c6ccccc65)cc4C#N)cc3)c2c1 ; N#Cc1cc(-c2ccc(-n3c4ccccc4c4c5oc6ccccc6c5ccc43)cc2)cc(-n2c3ccccc3c3cc(C#N)ccc32)c1]

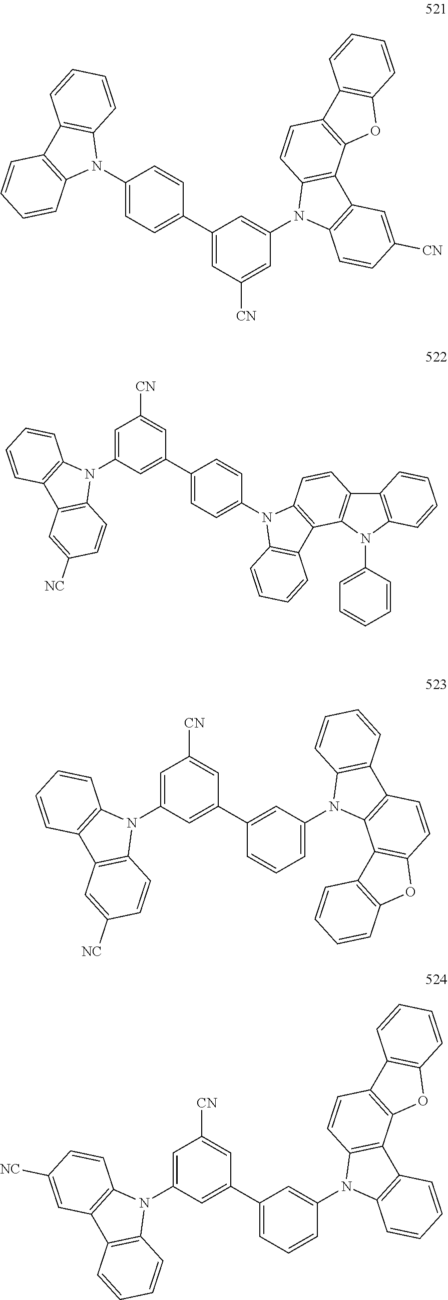 [SMILES: N#Cc1cc(-c2ccc(-n3c4ccccc4c4ccccc43)cc2)cc(-n2c3ccc(C#N)cc3c3c4oc5ccccc5c4ccc32)c1 ; N#Cc1cc(-c2ccc(-n3c4ccccc4c4c3ccc3c5ccccc5n(-c5ccccc5)c34)cc2)cc(-n2c3ccccc3c3cc(C#N)ccc32)c1 ; N#Cc1cc(-c2cccc(-n3c4ccccc4c4ccc5oc6ccccc6c5c43)c2)cc(-n2c3ccccc3c3cc(C#N)ccc32)c1 ; N#Cc1cc(-c2cccc(-n3c4ccccc4c4c5oc6ccccc6c5ccc43)c2)cc(-n2c3ccccc3c3cc(C#N)ccc32)c1]

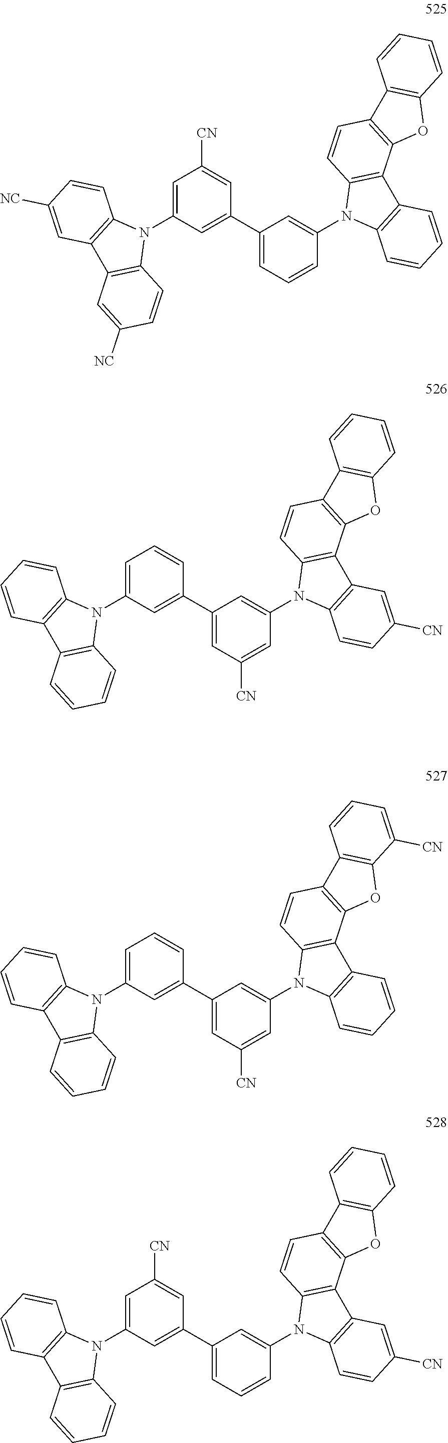 [SMILES: N#Cc1cc(-c2cccc(-n3c4ccccc4c4c5oc6ccccc6c5ccc43)c2)cc(-n2c3ccc(C#N)cc3c3cc(C#N)ccc32)c1 ; N#Cc1cc(-c2cccc(-n3c4ccccc4c4ccccc43)c2)cc(-n2c3ccc(C#N)cc3c3c4oc5ccccc5c4ccc32)c1 ; N#Cc1cc(-c2cccc(-n3c4ccccc4c4ccccc43)c2)cc(-n2c3ccccc3c3c4oc5c(C#N)cccc5c4ccc32)c1 ; N#Cc1cc(-c2cccc(-n3c4ccc(C#N)cc4c4c5oc6ccccc6c5ccc43)c2)cc(-n2c3ccccc3c3ccccc32)c1]

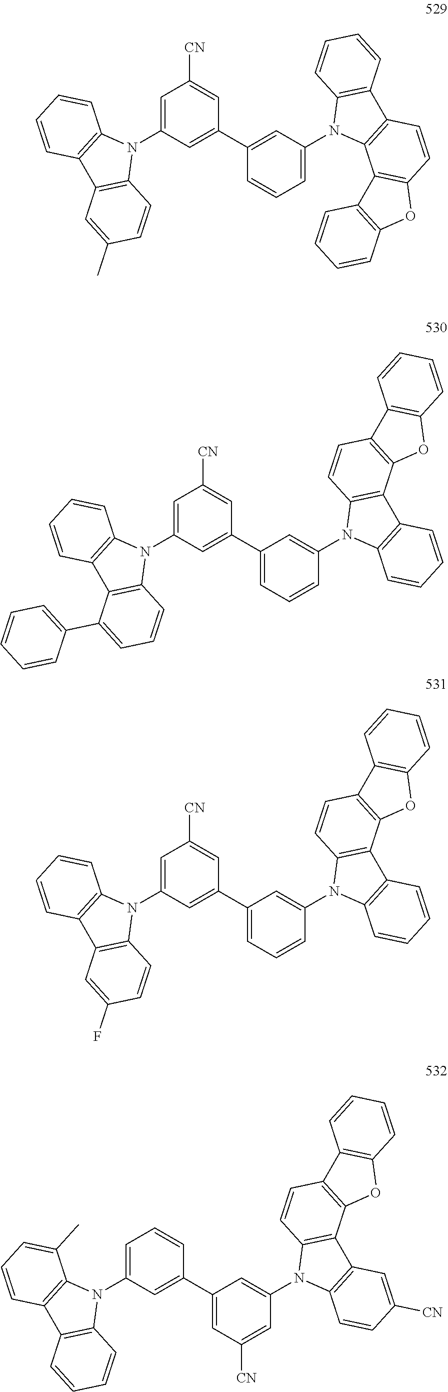 [SMILES: Cc1ccc2c(c1)c1ccccc1n2-c1cc(C#N)cc(-c2cccc(-n3c4ccccc4c4ccc5oc6ccccc6c5c43)c2)c1 ; N#Cc1cc(-c2cccc(-n3c4ccccc4c4c5oc6ccccc6c5ccc43)c2)cc(-n2c3ccccc3c3c(-c4ccccc4)cccc32)c1 ; N#Cc1cc(-c2cccc(-n3c4ccccc4c4c5oc6ccccc6c5ccc43)c2)cc(-n2c3ccccc3c3cc(F)ccc32)c1 ; Cc1cccc2c3ccccc3n(-c3cccc(-c4cc(C#N)cc(-n5c6ccc(C#N)cc6c6c7oc8ccccc8c7ccc65)c4)c3)c12]

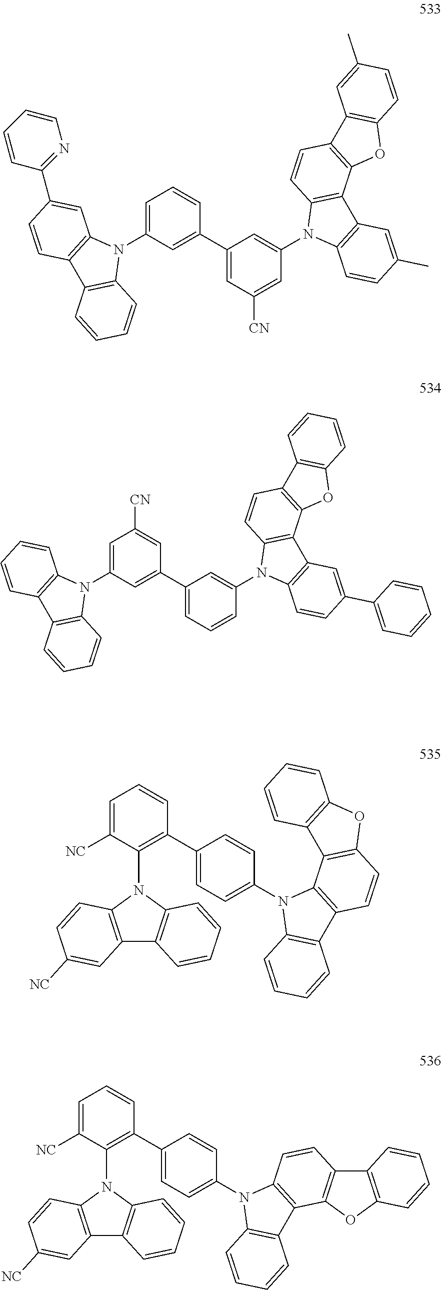 [SMILES: Cc1ccc2oc3c(ccc4c3c3cc(C)ccc3n4-c3cc(C#N)cc(-c4cccc(-n5c6ccccc6c6ccc(-c7ccccn7)cc65)c4)c3)c2c1 ; N#Cc1cc(-c2cccc(-n3c4ccc(-c5ccccc5)cc4c4c5oc6ccccc6c5ccc43)c2)cc(-n2c3ccccc3c3ccccc32)c1 ; N#Cc1ccc2c(c1)c1ccccc1n2-c1c(C#N)cccc1-c1ccc(-n2c3ccccc3c3ccc4oc5ccccc5c4c32)cc1 ; N#Cc1ccc2c(c1)c1ccccc1n2-c1c(C#N)cccc1-c1ccc(-n2c3ccccc3c3c4oc5ccccc5c4ccc32)cc1]

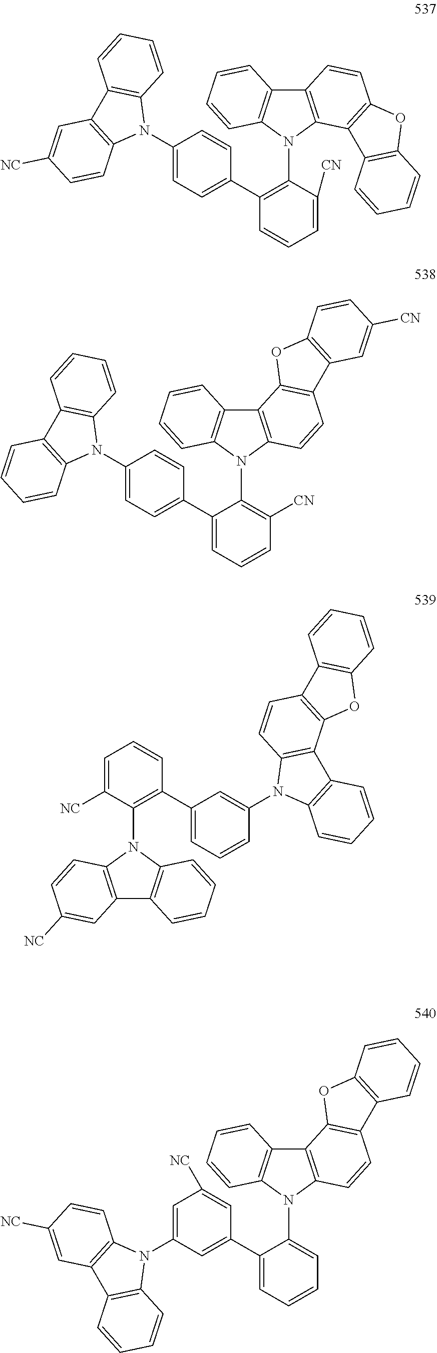 [SMILES: N#Cc1ccc2c(c1)c1ccccc1n2-c1ccc(-c2cccc(C#N)c2-n2c3ccccc3c3ccc4oc5ccccc5c4c32)cc1 ; N#Cc1ccc2oc3c(ccc4c3c3ccccc3n4-c3c(C#N)cccc3-c3ccc(-n4c5ccccc5c5ccccc54)cc3)c2c1 ; N#Cc1ccc2c(c1)c1ccccc1n2-c1c(C#N)cccc1-c1cccc(-n2c3ccccc3c3c4oc5ccccc5c4ccc32)c1 ; N#Cc1cc(-c2ccccc2-n2c3ccccc3c3c4oc5ccccc5c4ccc32)cc(-n2c3ccccc3c3cc(C#N)ccc32)c1]

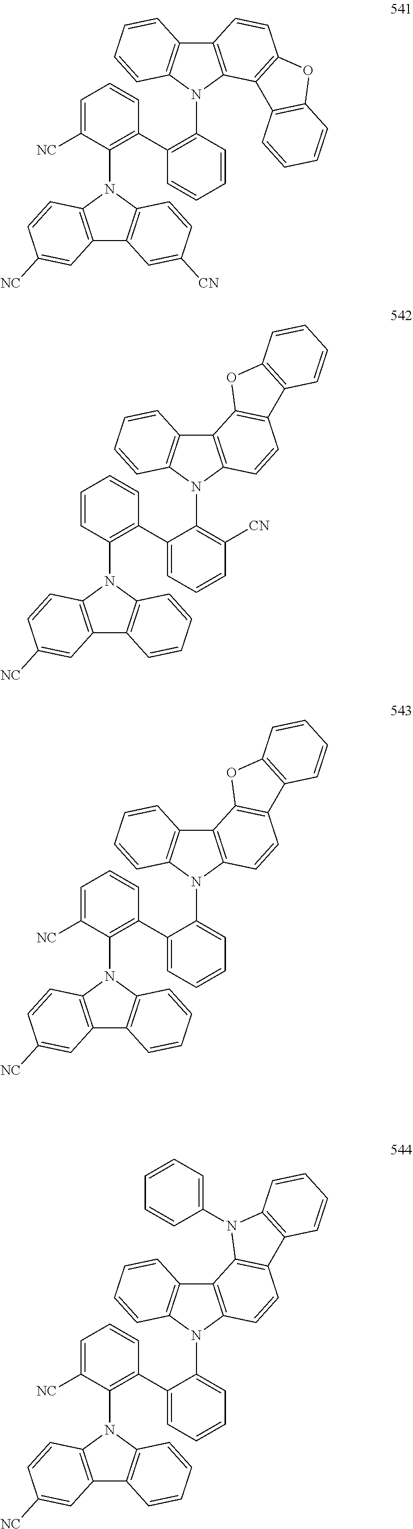 [SMILES: N#Cc1ccc2c(c1)c1cc(C#N)ccc1n2-c1c(C#N)cccc1-c1ccccc1-n1c2ccccc2c2ccc3oc4ccccc4c3c21 ; N#Cc1ccc2c(c1)c1ccccc1n2-c1ccccc1-c1cccc(C#N)c1-n1c2ccccc2c2c3oc4ccccc4c3ccc21 ; N#Cc1ccc2c(c1)c1ccccc1n2-c1c(C#N)cccc1-c1ccccc1-n1c2ccccc2c2c3oc4ccccc4c3ccc21 ; N#Cc1ccc2c(c1)c1ccccc1n2-c1c(C#N)cccc1-c1ccccc1-n1c2ccccc2c2c1ccc1c3ccccc3n(-c3ccccc3)c12]

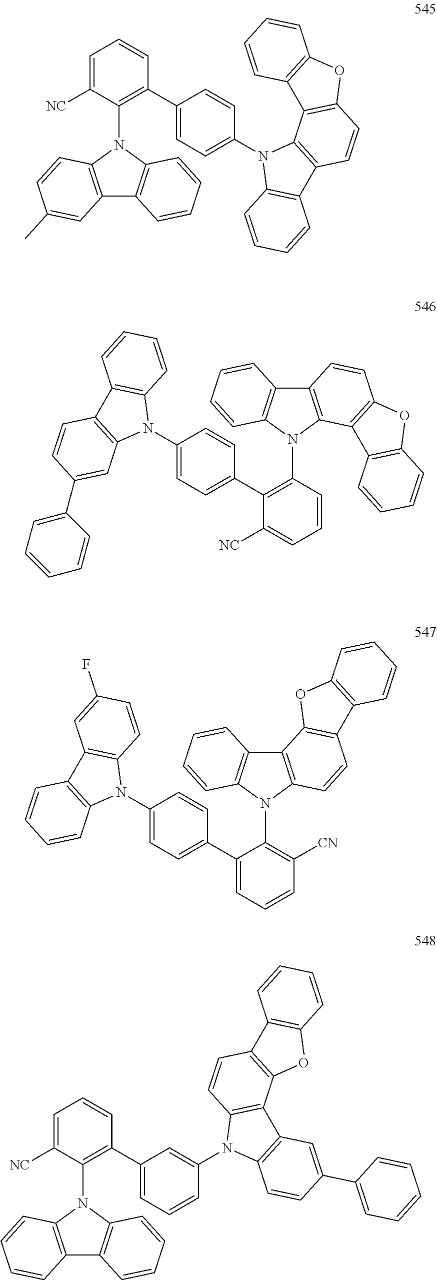 [SMILES: Cc1ccc2c(c1)c1ccccc1n2-c1c(C#N)cccc1-c1ccc(-n2c3ccccc3c3ccc4oc5ccccc5c4c32)cc1 ; N#Cc1cccc(-n2c3ccccc3c3ccc4oc5ccccc5c4c32)c1-c1ccc(-n2c3ccccc3c3ccc(-c4ccccc4)cc32)cc1 ; N#Cc1cccc(-c2ccc(-n3c4ccccc4c4cc(F)ccc43)cc2)c1-n1c2ccccc2c2c3oc4ccccc4c3ccc21 ; N#Cc1cccc(-c2cccc(-n3c4ccc(-c5ccccc5)cc4c4c5oc6ccccc6c5ccc43)c2)c1-n1c2ccccc2c2ccccc21]

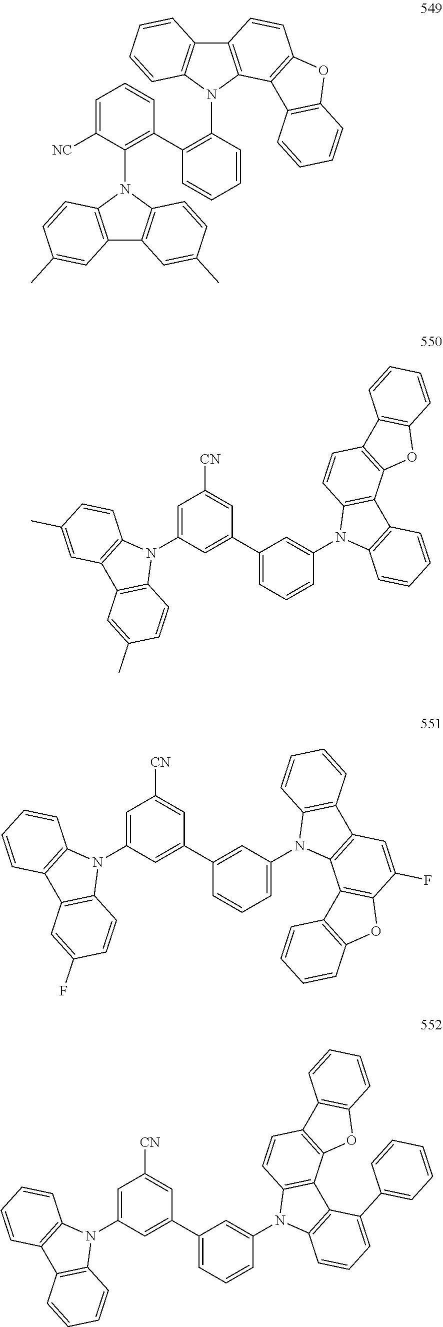 [SMILES: Cc1ccc2c(c1)c1cc(C)ccc1n2-c1c(C#N)cccc1-c1ccccc1-n1c2ccccc2c2ccc3oc4ccccc4c3c21 ; Cc1ccc2c(c1)c1cc(C)ccc1n2-c1cc(C#N)cc(-c2cccc(-n3c4ccccc4c4c5oc6ccccc6c5ccc43)c2)c1 ; N#Cc1cc(-c2cccc(-n3c4ccccc4c4cc(F)c5oc6ccccc6c5c43)c2)cc(-n2c3ccccc3c3cc(F)ccc32)c1 ; N#Cc1cc(-c2cccc(-n3c4cccc(-c5ccccc5)c4c4c5oc6ccccc6c5ccc43)c2)cc(-n2c3ccccc3c3ccccc32)c1]

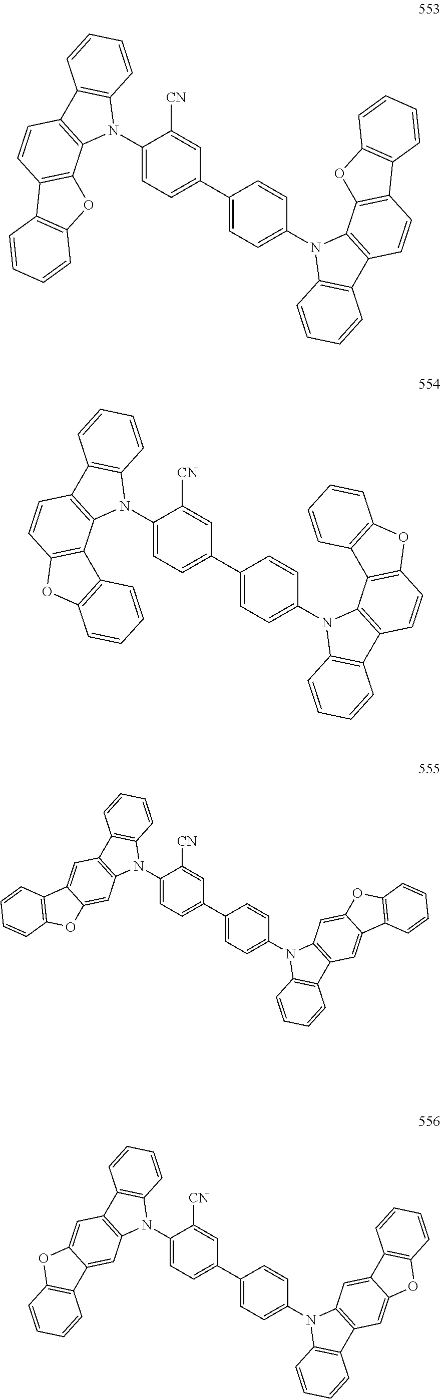 [SMILES: N#Cc1cc(-c2ccc(-n3c4ccccc4c4ccc5c6ccccc6oc5c43)cc2)ccc1-n1c2ccccc2c2ccc3c4ccccc4oc3c21 ; N#Cc1cc(-c2ccc(-n3c4ccccc4c4ccc5oc6ccccc6c5c43)cc2)ccc1-n1c2ccccc2c2ccc3oc4ccccc4c3c21 ; N#Cc1cc(-c2ccc(-n3c4ccccc4c4cc5c(cc43)oc3ccccc35)cc2)ccc1-n1c2ccccc2c2cc3c(cc21)oc1ccccc13 ; N#Cc1cc(-c2ccc(-n3c4ccccc4c4cc5oc6ccccc6c5cc43)cc2)ccc1-n1c2ccccc2c2cc3oc4ccccc4c3cc21]

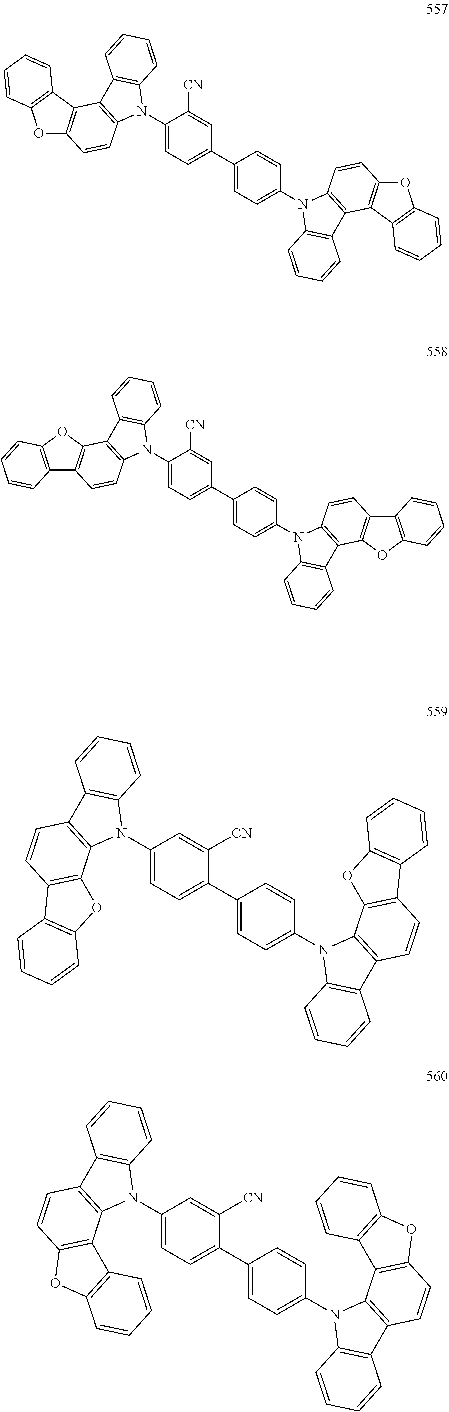 [SMILES: N#Cc1cc(-c2ccc(-n3c4ccccc4c4c5c(ccc43)oc3ccccc35)cc2)ccc1-n1c2ccccc2c2c3c(ccc21)oc1ccccc13 ; N#Cc1cc(-c2ccc(-n3c4ccccc4c4c5oc6ccccc6c5ccc43)cc2)ccc1-n1c2ccccc2c2c3oc4ccccc4c3ccc21 ; N#Cc1cc(-n2c3ccccc3c3ccc4c5ccccc5oc4c32)ccc1-c1ccc(-n2c3ccccc3c3ccc4c5ccccc5oc4c32)cc1 ; N#Cc1cc(-n2c3ccccc3c3ccc4oc5ccccc5c4c32)ccc1-c1ccc(-n2c3ccccc3c3ccc4oc5ccccc5c4c32)cc1]

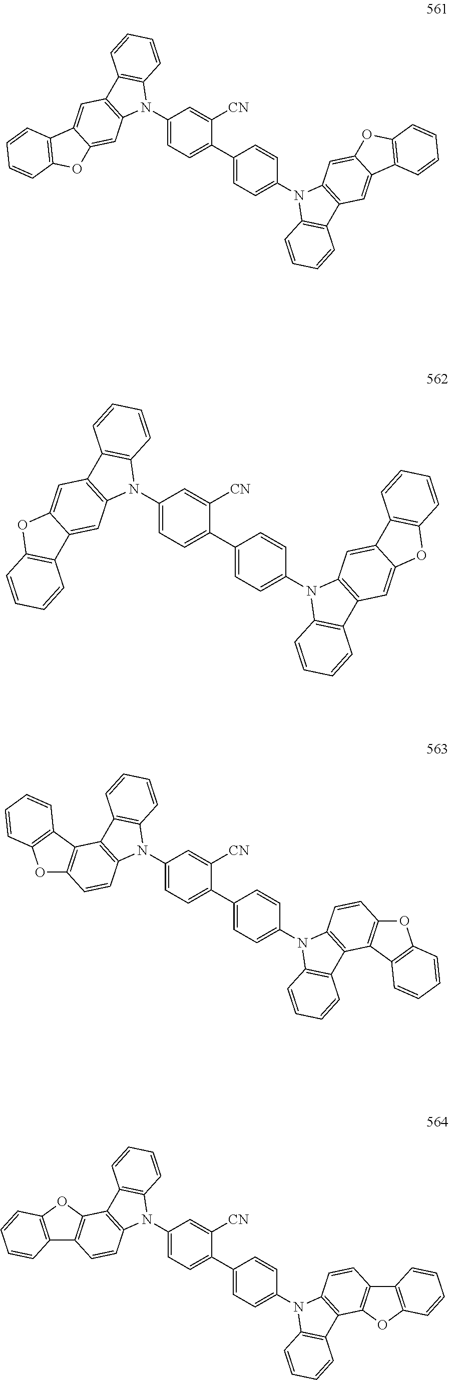 [SMILES: N#Cc1cc(-n2c3ccccc3c3cc4c(cc32)oc2ccccc24)ccc1-c1ccc(-n2c3ccccc3c3cc4c(cc32)oc2ccccc24)cc1 ; N#Cc1cc(-n2c3ccccc3c3cc4oc5ccccc5c4cc32)ccc1-c1ccc(-n2c3ccccc3c3cc4oc5ccccc5c4cc32)cc1 ; N#Cc1cc(-n2c3ccccc3c3c4c(ccc32)oc2ccccc24)ccc1-c1ccc(-n2c3ccccc3c3c4c(ccc32)oc2ccccc24)cc1 ; N#Cc1cc(-n2c3ccccc3c3c4oc5ccccc5c4ccc32)ccc1-c1ccc(-n2c3ccccc3c3c4oc5ccccc5c4ccc32)cc1]

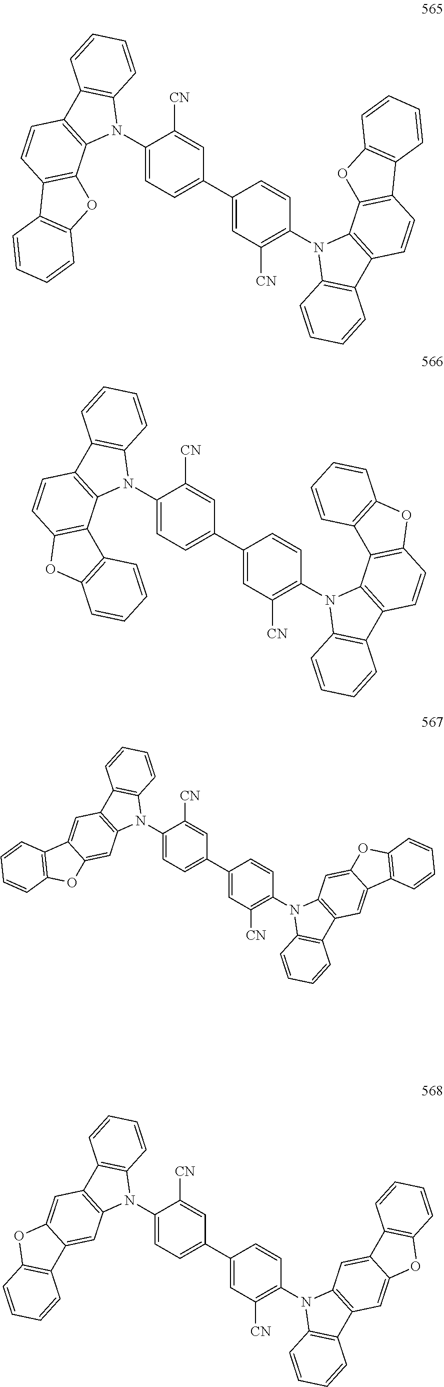 [SMILES: N#Cc1cc(-c2ccc(-n3c4ccccc4c4ccc5c6ccccc6oc5c43)c(C#N)c2)ccc1-n1c2ccccc2c2ccc3c4ccccc4oc3c21 ; N#Cc1cc(-c2ccc(-n3c4ccccc4c4ccc5oc6ccccc6c5c43)c(C#N)c2)ccc1-n1c2ccccc2c2ccc3oc4ccccc4c3c21 ; N#Cc1cc(-c2ccc(-n3c4ccccc4c4cc5c(cc43)oc3ccccc35)c(C#N)c2)ccc1-n1c2ccccc2c2cc3c(cc21)oc1ccccc13 ; N#Cc1cc(-c2ccc(-n3c4ccccc4c4cc5oc6ccccc6c5cc43)c(C#N)c2)ccc1-n1c2ccccc2c2cc3oc4ccccc4c3cc21]

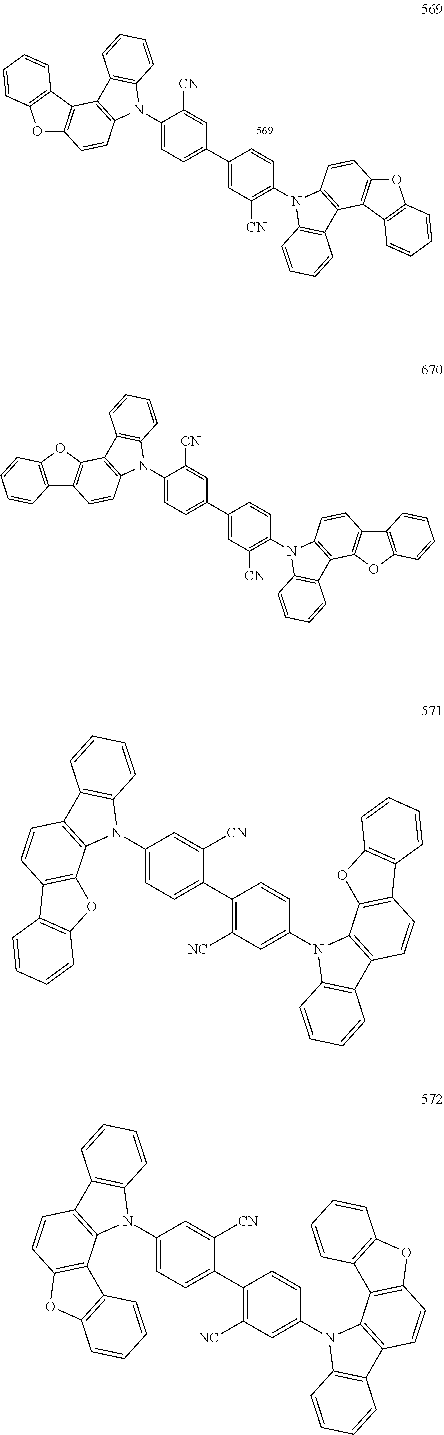 [SMILES: N#Cc1cc(-c2ccc(-n3c4ccccc4c4c5c(ccc43)oc3ccccc35)c(C#N)c2)ccc1-n1c2ccccc2c2c3c(ccc21)oc1ccccc13 ; N#Cc1cc(-c2ccc(-n3c4ccccc4c4c5oc6ccccc6c5ccc43)c(C#N)c2)ccc1-n1c2ccccc2c2c3oc4ccccc4c3ccc21 ; N#Cc1cc(-n2c3ccccc3c3ccc4c5ccccc5oc4c32)ccc1-c1ccc(-n2c3ccccc3c3ccc4c5ccccc5oc4c32)cc1C#N ; N#Cc1cc(-n2c3ccccc3c3ccc4oc5ccccc5c4c32)ccc1-c1ccc(-n2c3ccccc3c3ccc4oc5ccccc5c4c32)cc1C#N]

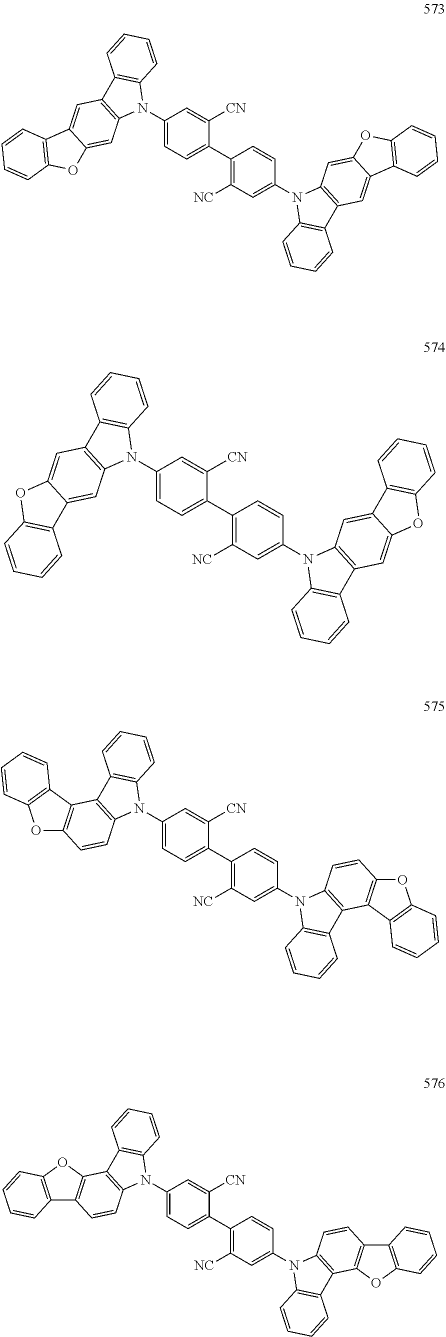 [SMILES: N#Cc1cc(-n2c3ccccc3c3cc4c(cc32)oc2ccccc24)ccc1-c1ccc(-n2c3ccccc3c3cc4c(cc32)oc2ccccc24)cc1C#N ; N#Cc1cc(-n2c3ccccc3c3cc4oc5ccccc5c4cc32)ccc1-c1ccc(-n2c3ccccc3c3cc4oc5ccccc5c4cc32)cc1C#N ; N#Cc1cc(-n2c3ccccc3c3c4c(ccc32)oc2ccccc24)ccc1-c1ccc(-n2c3ccccc3c3c4c(ccc32)oc2ccccc24)cc1C#N ; N#Cc1cc(-n2c3ccccc3c3c4oc5ccccc5c4ccc32)ccc1-c1ccc(-n2c3ccccc3c3c4oc5ccccc5c4ccc32)cc1C#N]

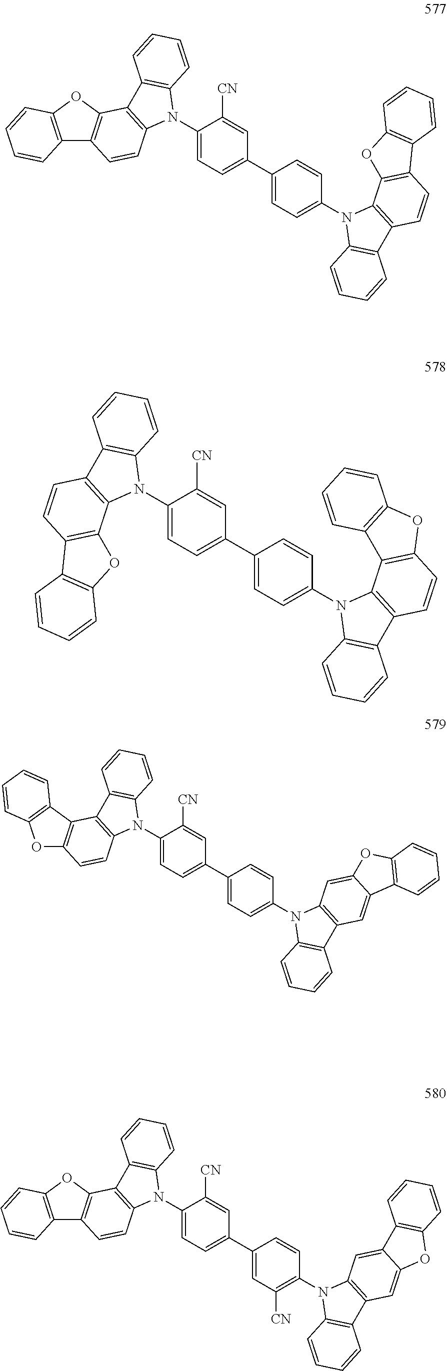 [SMILES: N#Cc1cc(-c2ccc(-n3c4ccccc4c4ccc5c6ccccc6oc5c43)cc2)ccc1-n1c2ccccc2c2c3oc4ccccc4c3ccc21 ; N#Cc1cc(-c2ccc(-n3c4ccccc4c4ccc5oc6ccccc6c5c43)cc2)ccc1-n1c2ccccc2c2ccc3c4ccccc4oc3c21 ; N#Cc1cc(-c2ccc(-n3c4ccccc4c4cc5c(cc43)oc3ccccc35)cc2)ccc1-n1c2ccccc2c2c3c(ccc21)oc1ccccc13 ; N#Cc1cc(-c2ccc(-n3c4ccccc4c4c5oc6ccccc6c5ccc43)c(C#N)c2)ccc1-n1c2ccccc2c2cc3oc4ccccc4c3cc21]

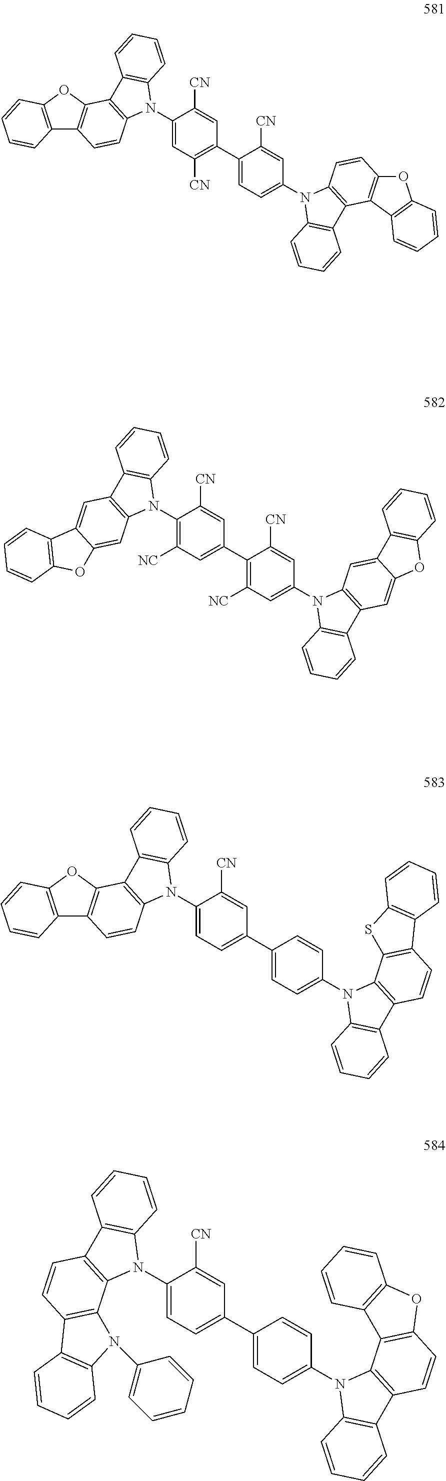 [SMILES: N#Cc1cc(-n2c3ccccc3c3c4c(ccc32)oc2ccccc24)ccc1-c1cc(C#N)c(-n2c3ccccc3c3c4oc5ccccc5c4ccc32)cc1C#N ; N#Cc1cc(-n2c3ccccc3c3cc4oc5ccccc5c4cc32)cc(C#N)c1-c1cc(C#N)c(-n2c3ccccc3c3cc4c(cc32)oc2ccccc24)c(C#N)c1 ; N#Cc1cc(-c2ccc(-n3c4ccccc4c4ccc5c6ccccc6sc5c43)cc2)ccc1-n1c2ccccc2c2c3oc4ccccc4c3ccc21 ; N#Cc1cc(-c2ccc(-n3c4ccccc4c4ccc5oc6ccccc6c5c43)cc2)ccc1-n1c2ccccc2c2ccc3c4ccccc4n(-c4ccccc4)c3c21]

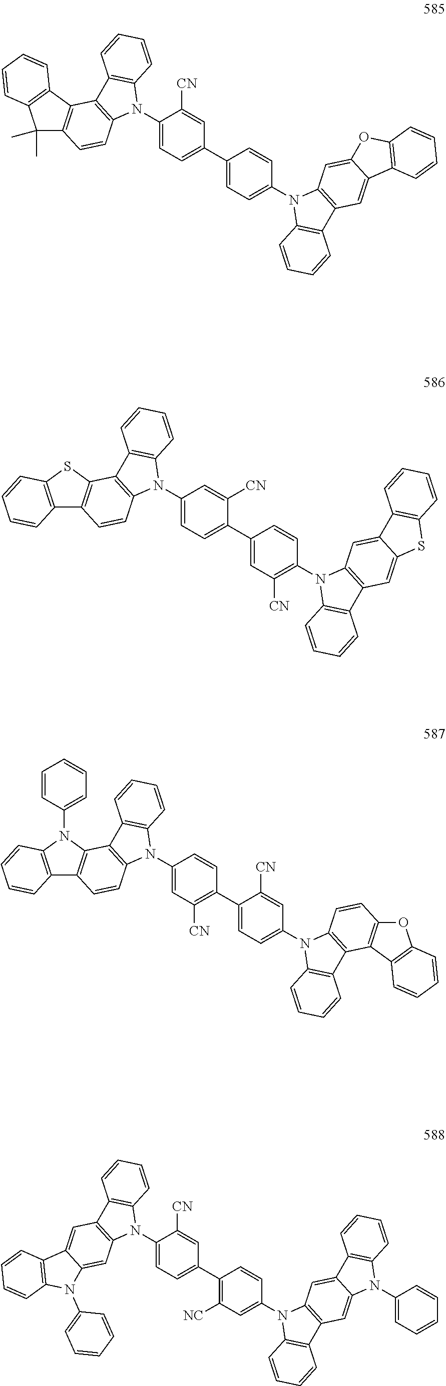 [SMILES: CC1(C)c2ccccc2-c2c1ccc1c2c2ccccc2n1-c1ccc(-c2ccc(-n3c4ccccc4c4cc5c(cc43)oc3ccccc35)cc2)cc1C#N ; N#Cc1cc(-n2c3ccccc3c3c4sc5ccccc5c4ccc32)ccc1-c1ccc(-n2c3ccccc3c3cc4sc5ccccc5c4cc32)c(C#N)c1 ; N#Cc1cc(-n2c3ccccc3c3c4c(ccc32)oc2ccccc24)ccc1-c1ccc(-n2c3ccccc3c3c2ccc2c4ccccc4n(-c4ccccc4)c23)cc1C#N ; N#Cc1cc(-n2c3ccccc3c3cc4c(cc32)c2ccccc2n4-c2ccccc2)ccc1-c1ccc(-n2c3ccccc3c3cc4c5ccccc5n(-c5ccccc5)c4cc32)c(C#N)c1]

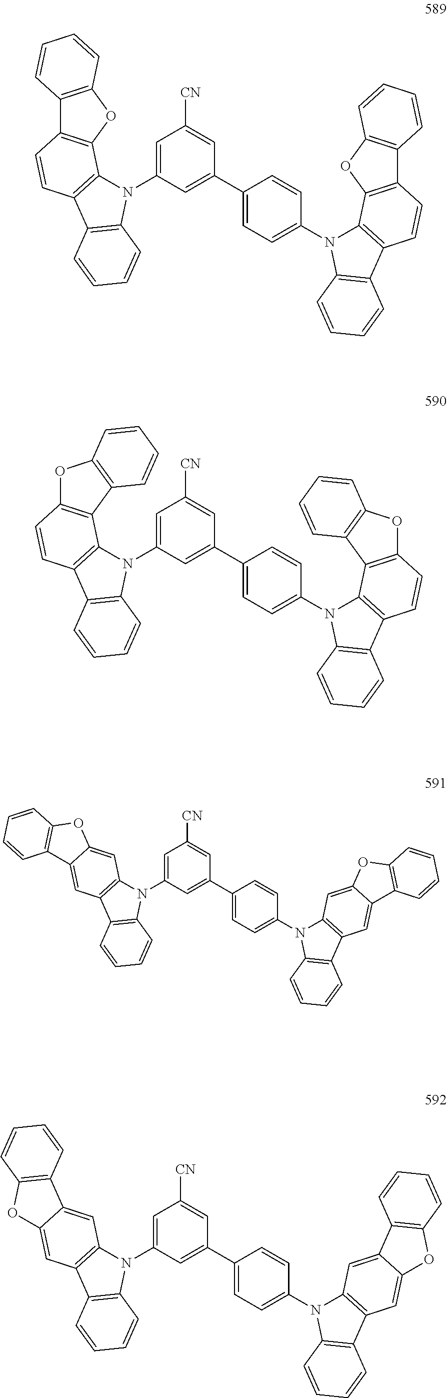 [SMILES: N#Cc1cc(-c2ccc(-n3c4ccccc4c4ccc5c6ccccc6oc5c43)cc2)cc(-n2c3ccccc3c3ccc4c5ccccc5oc4c32)c1 ; N#Cc1cc(-c2ccc(-n3c4ccccc4c4ccc5oc6ccccc6c5c43)cc2)cc(-n2c3ccccc3c3ccc4oc5ccccc5c4c32)c1 ; N#Cc1cc(-c2ccc(-n3c4ccccc4c4cc5c(cc43)oc3ccccc35)cc2)cc(-n2c3ccccc3c3cc4c(cc32)oc2ccccc24)c1 ; N#Cc1cc(-c2ccc(-n3c4ccccc4c4cc5oc6ccccc6c5cc43)cc2)cc(-n2c3ccccc3c3cc4oc5ccccc5c4cc32)c1]

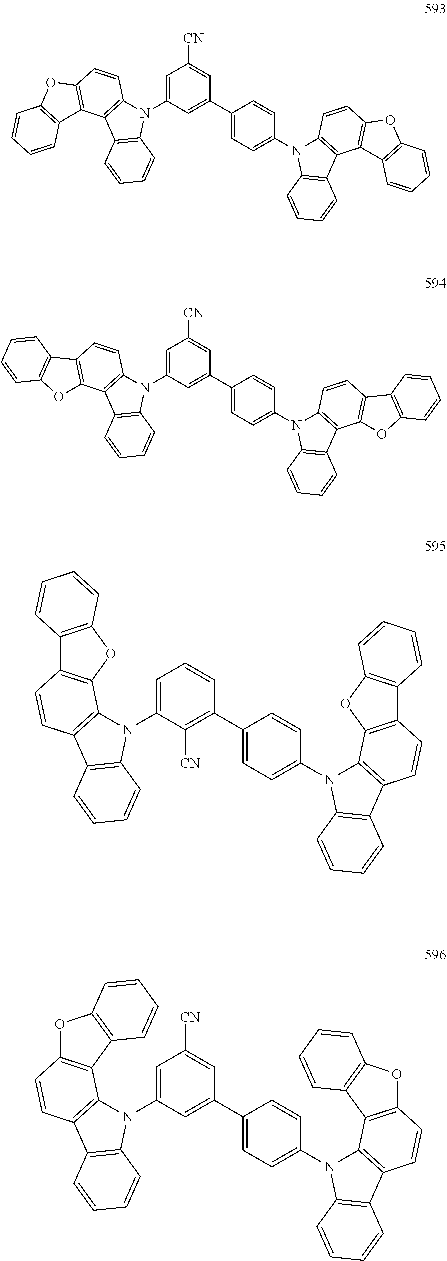 [SMILES: N#Cc1cc(-c2ccc(-n3c4ccccc4c4c5c(ccc43)oc3ccccc35)cc2)cc(-n2c3ccccc3c3c4c(ccc32)oc2ccccc24)c1 ; N#Cc1cc(-c2ccc(-n3c4ccccc4c4c5oc6ccccc6c5ccc43)cc2)cc(-n2c3ccccc3c3c4oc5ccccc5c4ccc32)c1 ; N#Cc1c(-c2ccc(-n3c4ccccc4c4ccc5c6ccccc6oc5c43)cc2)cccc1-n1c2ccccc2c2ccc3c4ccccc4oc3c21 ; N#Cc1cc(-c2ccc(-n3c4ccccc4c4ccc5oc6ccccc6c5c43)cc2)cc(-n2c3ccccc3c3ccc4oc5ccccc5c4c32)c1]

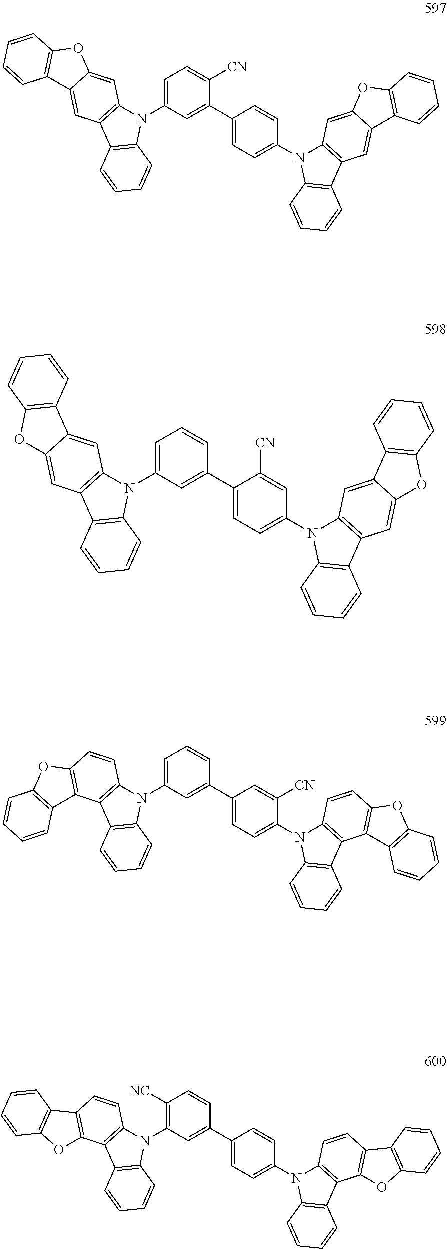 [SMILES: N#Cc1ccc(-n2c3ccccc3c3cc4c(cc32)oc2ccccc24)cc1-c1ccc(-n2c3ccccc3c3cc4c(cc32)oc2ccccc24)cc1 ; N#Cc1cc(-n2c3ccccc3c3cc4oc5ccccc5c4cc32)ccc1-c1cccc(-n2c3ccccc3c3cc4oc5ccccc5c4cc32)c1 ; N#Cc1cc(-c2cccc(-n3c4ccccc4c4c5c(ccc43)oc3ccccc35)c2)ccc1-n1c2ccccc2c2c3c(ccc21)oc1ccccc13 ; N#Cc1ccc(-c2ccc(-n3c4ccccc4c4c5oc6ccccc6c5ccc43)cc2)cc1-n1c2ccccc2c2c3oc4ccccc4c3ccc21]

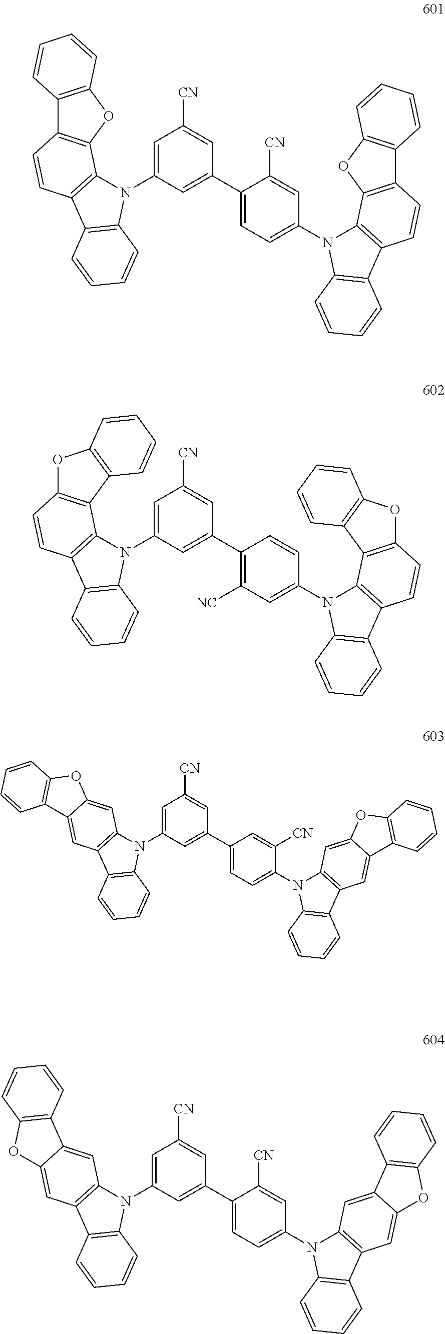 [SMILES: N#Cc1cc(-c2ccc(-n3c4ccccc4c4ccc5c6ccccc6oc5c43)cc2C#N)cc(-n2c3ccccc3c3ccc4c5ccccc5oc4c32)c1 ; N#Cc1cc(-c2ccc(-n3c4ccccc4c4ccc5oc6ccccc6c5c43)cc2C#N)cc(-n2c3ccccc3c3ccc4oc5ccccc5c4c32)c1 ; N#Cc1cc(-c2ccc(-n3c4ccccc4c4cc5c(cc43)oc3ccccc35)c(C#N)c2)cc(-n2c3ccccc3c3cc4c(cc32)oc2ccccc24)c1 ; N#Cc1cc(-c2ccc(-n3c4ccccc4c4cc5oc6ccccc6c5cc43)cc2C#N)cc(-n2c3ccccc3c3cc4oc5ccccc5c4cc32)c1]

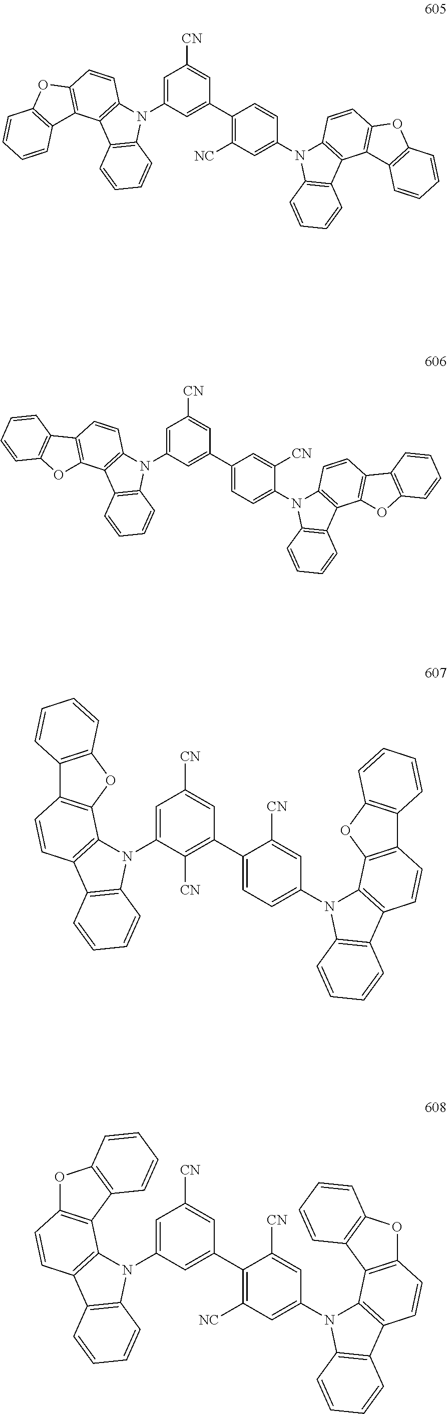 [SMILES: N#Cc1cc(-c2ccc(-n3c4ccccc4c4c5c(ccc43)oc3ccccc35)cc2C#N)cc(-n2c3ccccc3c3c4c(ccc32)oc2ccccc24)c1 ; N#Cc1cc(-c2ccc(-n3c4ccccc4c4c5oc6ccccc6c5ccc43)c(C#N)c2)cc(-n2c3ccccc3c3c4oc5ccccc5c4ccc32)c1 ; N#Cc1cc(-c2ccc(-n3c4ccccc4c4ccc5c6ccccc6oc5c43)cc2C#N)c(C#N)c(-n2c3ccccc3c3ccc4c5ccccc5oc4c32)c1 ; N#Cc1cc(-c2c(C#N)cc(-n3c4ccccc4c4ccc5oc6ccccc6c5c43)cc2C#N)cc(-n2c3ccccc3c3ccc4oc5ccccc5c4c32)c1]

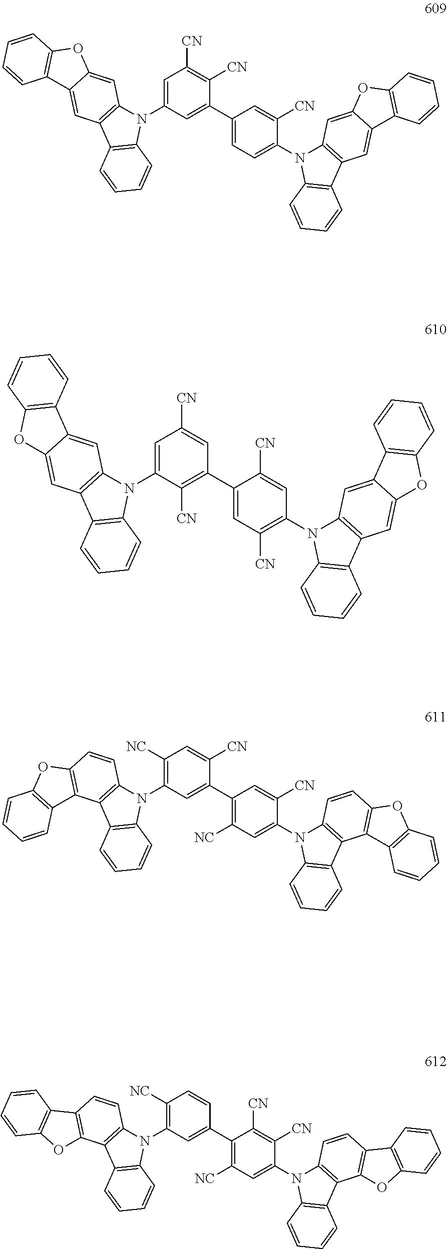 [SMILES: N#Cc1cc(-c2cc(-n3c4ccccc4c4cc5c(cc43)oc3ccccc35)cc(C#N)c2C#N)ccc1-n1c2ccccc2c2cc3c(cc21)oc1ccccc13 ; N#Cc1cc(-c2cc(C#N)c(-n3c4ccccc4c4cc5oc6ccccc6c5cc43)cc2C#N)c(C#N)c(-n2c3ccccc3c3cc4oc5ccccc5c4cc32)c1 ; N#Cc1cc(-n2c3ccccc3c3c4c(ccc32)oc2ccccc24)c(C#N)cc1-c1cc(-n2c3ccccc3c3c4c(ccc32)oc2ccccc24)c(C#N)cc1C#N ; N#Cc1ccc(-c2c(C#N)cc(-n3c4ccccc4c4c5oc6ccccc6c5ccc43)c(C#N)c2C#N)cc1-n1c2ccccc2c2c3oc4ccccc4c3ccc21]

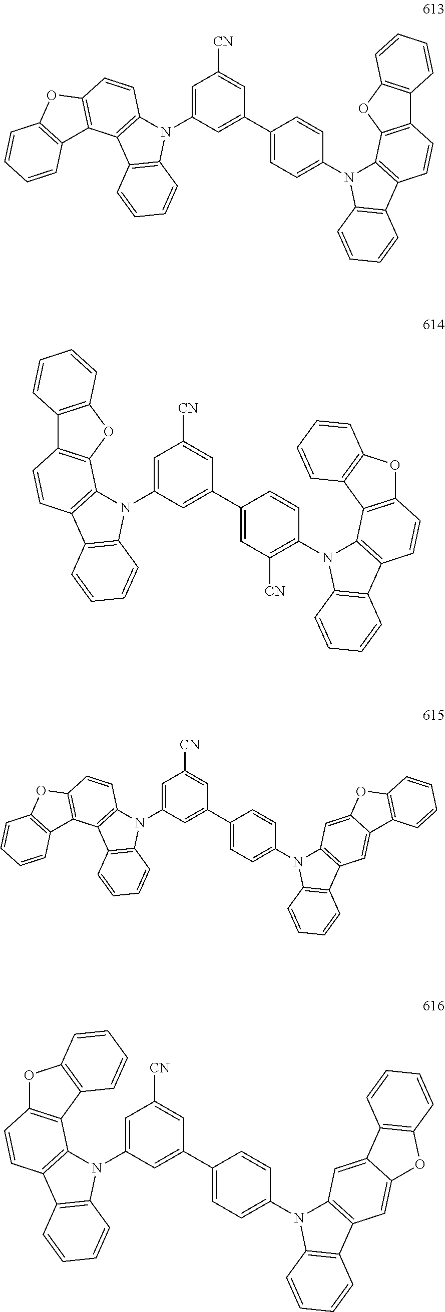 [SMILES: N#Cc1cc(-c2ccc(-n3c4ccccc4c4ccc5c6ccccc6oc5c43)cc2)cc(-n2c3ccccc3c3c4c(ccc32)oc2ccccc24)c1 ; N#Cc1cc(-c2ccc(-n3c4ccccc4c4ccc5oc6ccccc6c5c43)c(C#N)c2)cc(-n2c3ccccc3c3ccc4c5ccccc5oc4c32)c1 ; N#Cc1cc(-c2ccc(-n3c4ccccc4c4cc5c(cc43)oc3ccccc35)cc2)cc(-n2c3ccccc3c3c4c(ccc32)oc2ccccc24)c1 ; N#Cc1cc(-c2ccc(-n3c4ccccc4c4cc5oc6ccccc6c5cc43)cc2)cc(-n2c3ccccc3c3ccc4oc5ccccc5c4c32)c1]

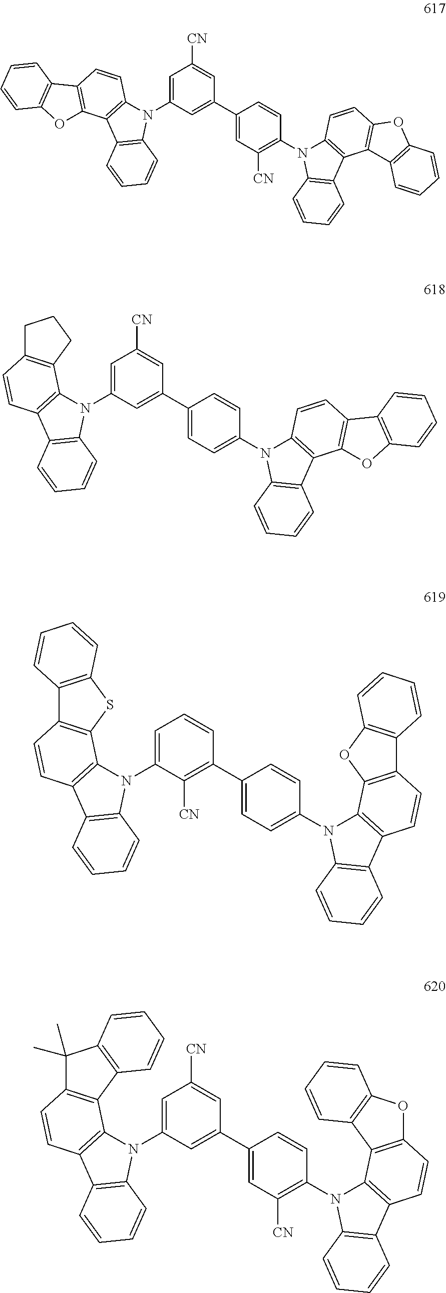 [SMILES: N#Cc1cc(-c2ccc(-n3c4ccccc4c4c5c(ccc43)oc3ccccc35)c(C#N)c2)cc(-n2c3ccccc3c3c4oc5ccccc5c4ccc32)c1 ; N#Cc1cc(-c2ccc(-n3c4ccccc4c4c5oc6ccccc6c5ccc43)cc2)cc(-n2c3ccccc3c3ccc4c(c32)CCC4)c1 ; N#Cc1c(-c2ccc(-n3c4ccccc4c4ccc5c6ccccc6oc5c43)cc2)cccc1-n1c2ccccc2c2ccc3c4ccccc4sc3c21 ; CC1(C)c2ccccc2-c2c1ccc1c3ccccc3n(-c3cc(C#N)cc(-c4ccc(-n5c6ccccc6c6ccc7oc8ccccc8c7c65)c(C#N)c4)c3)c21]

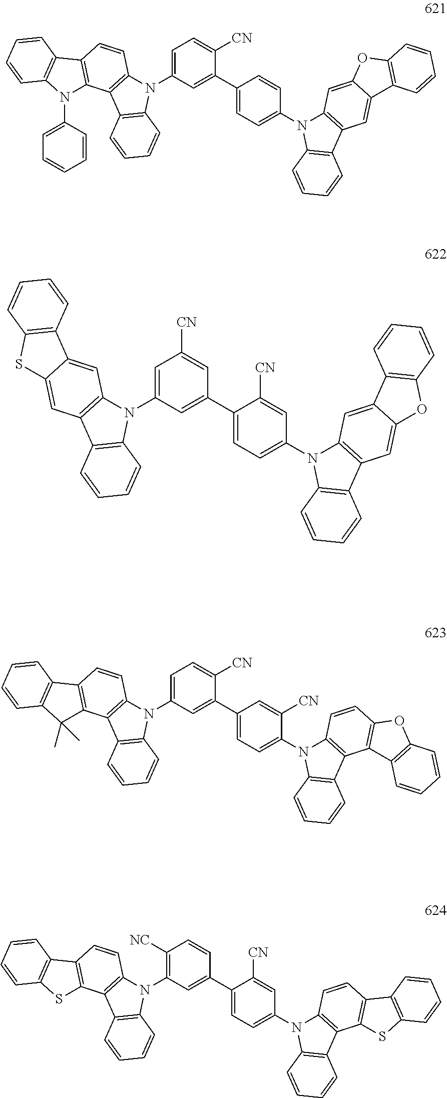 [SMILES: N#Cc1ccc(-n2c3ccccc3c3c2ccc2c4ccccc4n(-c4ccccc4)c23)cc1-c1ccc(-n2c3ccccc3c3cc4c(cc32)oc2ccccc24)cc1 ; N#Cc1cc(-c2ccc(-n3c4ccccc4c4cc5oc6ccccc6c5cc43)cc2C#N)cc(-n2c3ccccc3c3cc4sc5ccccc5c4cc32)c1 ; CC1(C)c2ccccc2-c2ccc3c(c21)c1ccccc1n3-c1ccc(C#N)c(-c2ccc(-n3c4ccccc4c4c5c(ccc43)oc3ccccc35)c(C#N)c2)c1 ; N#Cc1cc(-n2c3ccccc3c3c4sc5ccccc5c4ccc32)ccc1-c1ccc(C#N)c(-n2c3ccccc3c3c4sc5ccccc5c4ccc32)c1]

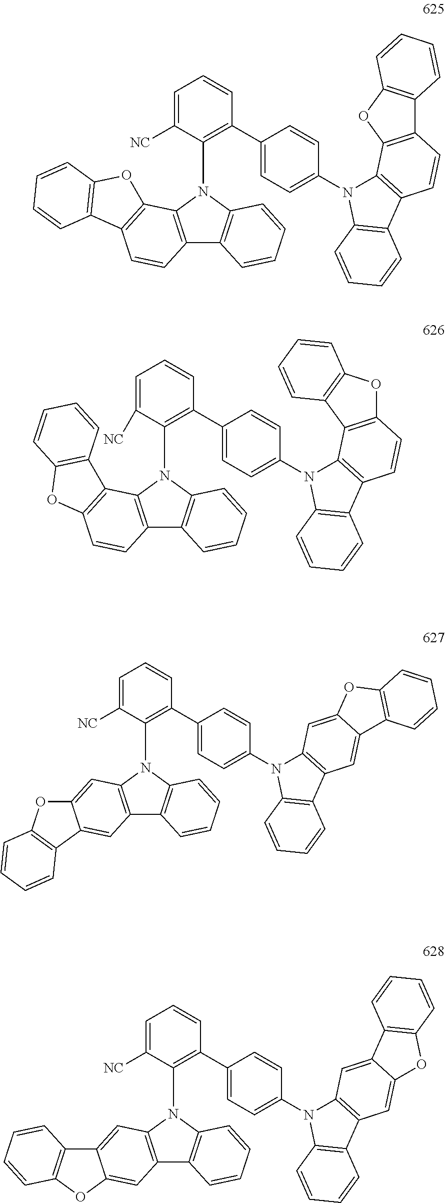 [SMILES: N#Cc1cccc(-c2ccc(-n3c4ccccc4c4ccc5c6ccccc6oc5c43)cc2)c1-n1c2ccccc2c2ccc3c4ccccc4oc3c21 ; N#Cc1cccc(-c2ccc(-n3c4ccccc4c4ccc5oc6ccccc6c5c43)cc2)c1-n1c2ccccc2c2ccc3oc4ccccc4c3c21 ; N#Cc1cccc(-c2ccc(-n3c4ccccc4c4cc5c(cc43)oc3ccccc35)cc2)c1-n1c2ccccc2c2cc3c(cc21)oc1ccccc13 ; N#Cc1cccc(-c2ccc(-n3c4ccccc4c4cc5oc6ccccc6c5cc43)cc2)c1-n1c2ccccc2c2cc3oc4ccccc4c3cc21]

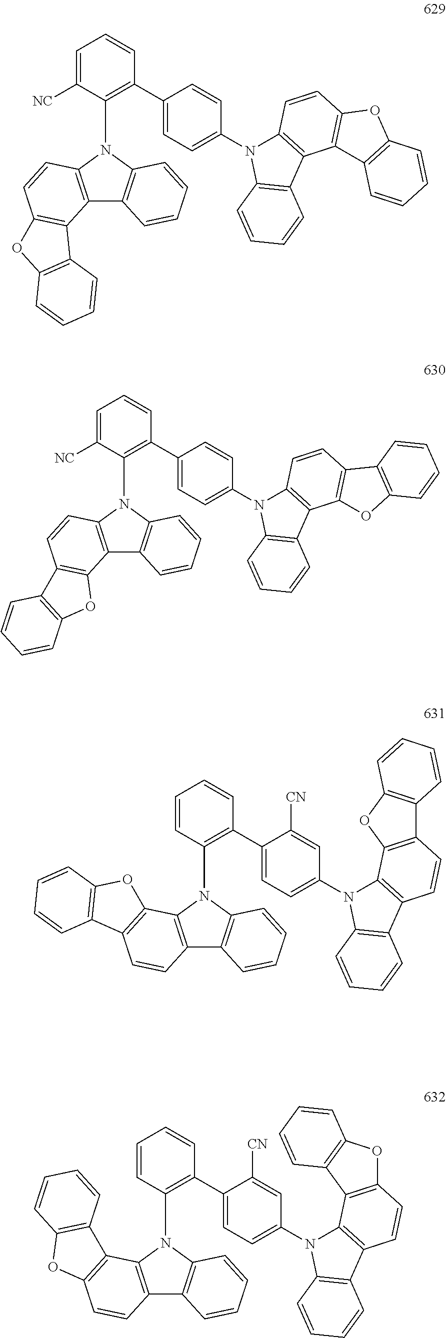 [SMILES: N#Cc1cccc(-c2ccc(-n3c4ccccc4c4c5c(ccc43)oc3ccccc35)cc2)c1-n1c2ccccc2c2c3c(ccc21)oc1ccccc13 ; N#Cc1cccc(-c2ccc(-n3c4ccccc4c4c5oc6ccccc6c5ccc43)cc2)c1-n1c2ccccc2c2c3oc4ccccc4c3ccc21 ; N#Cc1cc(-n2c3ccccc3c3ccc4c5ccccc5oc4c32)ccc1-c1ccccc1-n1c2ccccc2c2ccc3c4ccccc4oc3c21 ; N#Cc1cc(-n2c3ccccc3c3ccc4oc5ccccc5c4c32)ccc1-c1ccccc1-n1c2ccccc2c2ccc3oc4ccccc4c3c21]

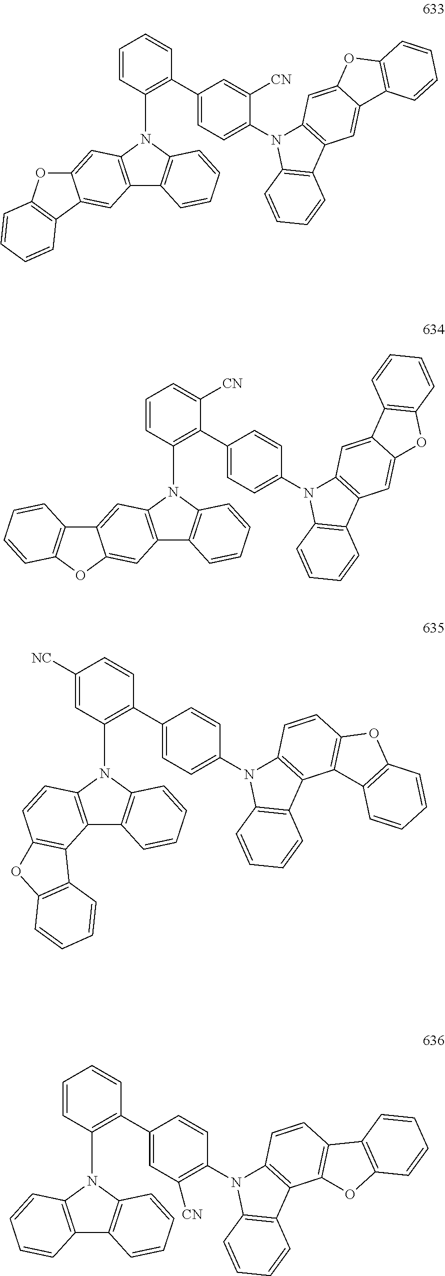 [SMILES: N#Cc1cc(-c2ccccc2-n2c3ccccc3c3cc4c(cc32)oc2ccccc24)ccc1-n1c2ccccc2c2cc3c(cc21)oc1ccccc13 ; N#Cc1cccc(-n2c3ccccc3c3cc4oc5ccccc5c4cc32)c1-c1ccc(-n2c3ccccc3c3cc4oc5ccccc5c4cc32)cc1 ; N#Cc1ccc(-c2ccc(-n3c4ccccc4c4c5c(ccc43)oc3ccccc35)cc2)c(-n2c3ccccc3c3c4c(ccc32)oc2ccccc24)c1 ; N#Cc1cc(-c2ccccc2-n2c3ccccc3c3ccccc32)ccc1-n1c2ccccc2c2c3oc4ccccc4c3ccc21]

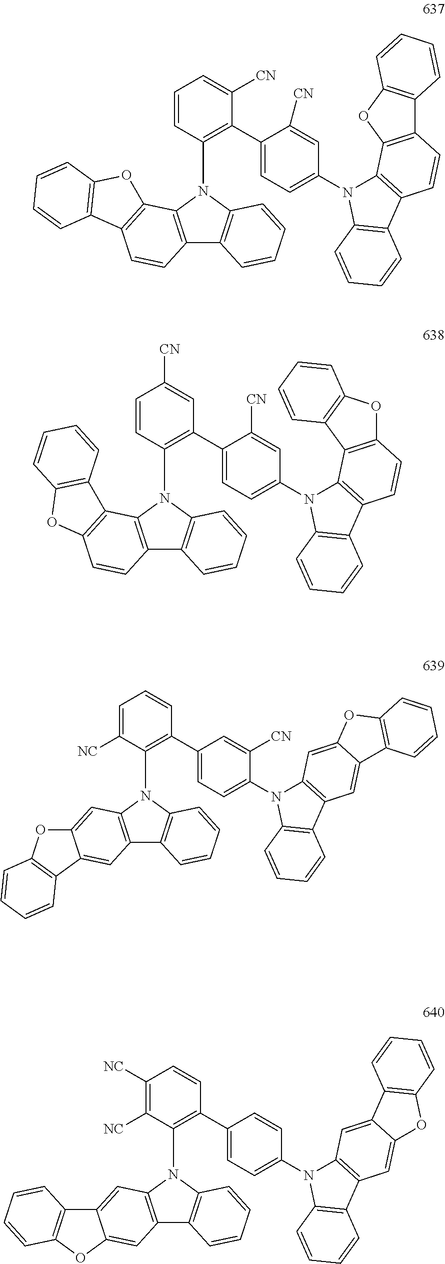 [SMILES: N#Cc1cc(-n2c3ccccc3c3ccc4c5ccccc5oc4c32)ccc1-c1c(C#N)cccc1-n1c2ccccc2c2ccc3c4ccccc4oc3c21 ; N#Cc1ccc(-n2c3ccccc3c3ccc4oc5ccccc5c4c32)c(-c2ccc(-n3c4ccccc4c4ccc5oc6ccccc6c5c43)cc2C#N)c1 ; N#Cc1cc(-c2cccc(C#N)c2-n2c3ccccc3c3cc4c(cc32)oc2ccccc24)ccc1-n1c2ccccc2c2cc3c(cc21)oc1ccccc13 ; N#Cc1ccc(-c2ccc(-n3c4ccccc4c4cc5oc6ccccc6c5cc43)cc2)c(-n2c3ccccc3c3cc4oc5ccccc5c4cc32)c1C#N]

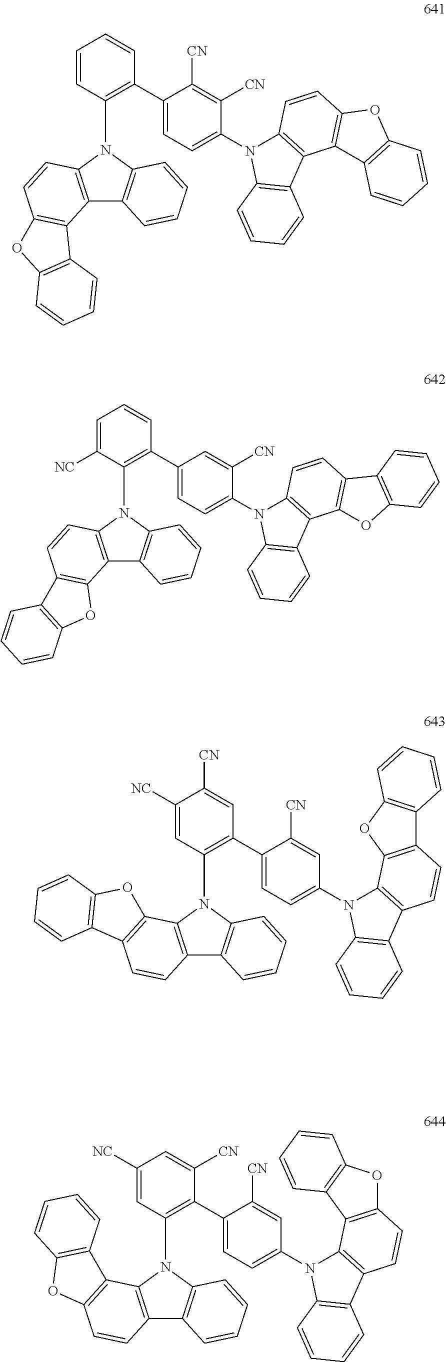 [SMILES: N#Cc1c(-c2ccccc2-n2c3ccccc3c3c4c(ccc32)oc2ccccc24)ccc(-n2c3ccccc3c3c4c(ccc32)oc2ccccc24)c1C#N ; N#Cc1cc(-c2cccc(C#N)c2-n2c3ccccc3c3c4oc5ccccc5c4ccc32)ccc1-n1c2ccccc2c2c3oc4ccccc4c3ccc21 ; N#Cc1cc(-c2ccc(-n3c4ccccc4c4ccc5c6ccccc6oc5c43)cc2C#N)c(-n2c3ccccc3c3ccc4c5ccccc5oc4c32)cc1C#N ; N#Cc1cc(C#N)c(-c2ccc(-n3c4ccccc4c4ccc5oc6ccccc6c5c43)cc2C#N)c(-n2c3ccccc3c3ccc4oc5ccccc5c4c32)c1]

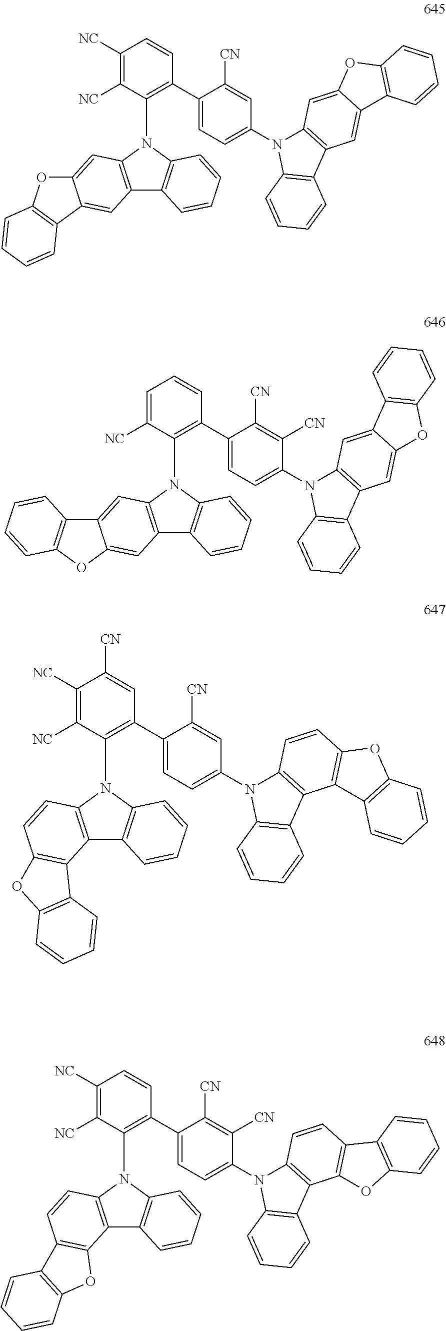 [SMILES: N#Cc1cc(-n2c3ccccc3c3cc4c(cc32)oc2ccccc24)ccc1-c1ccc(C#N)c(C#N)c1-n1c2ccccc2c2cc3c(cc21)oc1ccccc13 ; N#Cc1cccc(-c2ccc(-n3c4ccccc4c4cc5oc6ccccc6c5cc43)c(C#N)c2C#N)c1-n1c2ccccc2c2cc3oc4ccccc4c3cc21 ; N#Cc1cc(-n2c3ccccc3c3c4c(ccc32)oc2ccccc24)ccc1-c1cc(C#N)c(C#N)c(C#N)c1-n1c2ccccc2c2c3c(ccc21)oc1ccccc13 ; N#Cc1ccc(-c2ccc(-n3c4ccccc4c4c5oc6ccccc6c5ccc43)c(C#N)c2C#N)c(-n2c3ccccc3c3c4oc5ccccc5c4ccc32)c1C#N]

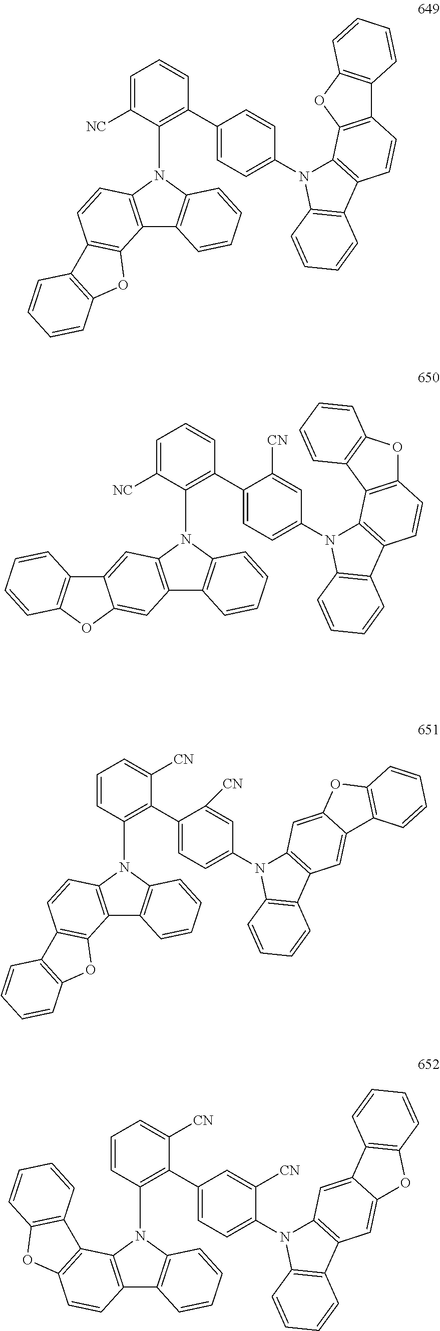 [SMILES: N#Cc1cccc(-c2ccc(-n3c4ccccc4c4ccc5c6ccccc6oc5c43)cc2)c1-n1c2ccccc2c2c3oc4ccccc4c3ccc21 ; N#Cc1cc(-n2c3ccccc3c3ccc4oc5ccccc5c4c32)ccc1-c1cccc(C#N)c1-n1c2ccccc2c2cc3oc4ccccc4c3cc21 ; N#Cc1cc(-n2c3ccccc3c3cc4c(cc32)oc2ccccc24)ccc1-c1c(C#N)cccc1-n1c2ccccc2c2c3oc4ccccc4c3ccc21 ; N#Cc1cc(-c2c(C#N)cccc2-n2c3ccccc3c3ccc4oc5ccccc5c4c32)ccc1-n1c2ccccc2c2cc3oc4ccccc4c3cc21]

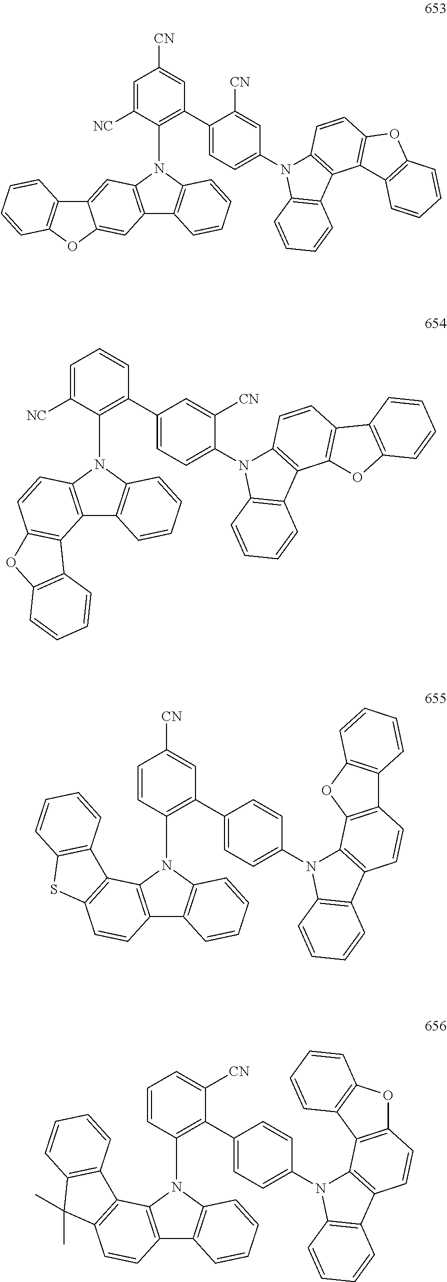 [SMILES: N#Cc1cc(C#N)c(-n2c3ccccc3c3cc4oc5ccccc5c4cc32)c(-c2ccc(-n3c4ccccc4c4c5c(ccc43)oc3ccccc35)cc2C#N)c1 ; N#Cc1cc(-c2cccc(C#N)c2-n2c3ccccc3c3c4c(ccc32)oc2ccccc24)ccc1-n1c2ccccc2c2c3oc4ccccc4c3ccc21 ; N#Cc1ccc(-n2c3ccccc3c3ccc4sc5ccccc5c4c32)c(-c2ccc(-n3c4ccccc4c4ccc5c6ccccc6oc5c43)cc2)c1 ; CC1(C)c2ccccc2-c2c1ccc1c3ccccc3n(-c3cccc(C#N)c3-c3ccc(-n4c5ccccc5c5ccc6oc7ccccc7c6c54)cc3)c21]

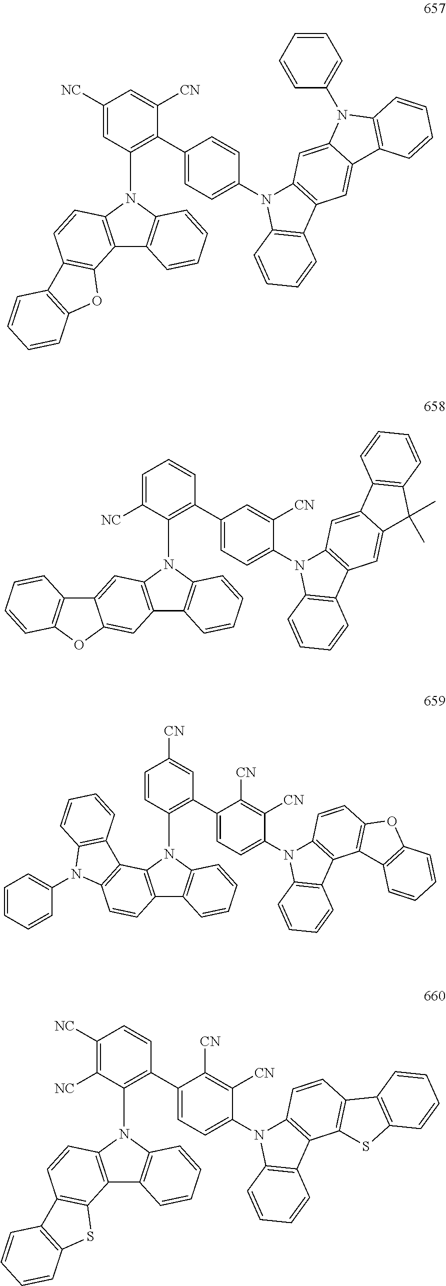 [SMILES: N#Cc1cc(C#N)c(-c2ccc(-n3c4ccccc4c4cc5c6ccccc6n(-c6ccccc6)c5cc43)cc2)c(-n2c3ccccc3c3c4oc5ccccc5c4ccc32)c1 ; CC1(C)c2ccccc2-c2cc3c(cc21)c1ccccc1n3-c1ccc(-c2cccc(C#N)c2-n2c3ccccc3c3cc4oc5ccccc5c4cc32)cc1C#N ; N#Cc1ccc(-n2c3ccccc3c3ccc4c(c5ccccc5n4-c4ccccc4)c32)c(-c2ccc(-n3c4ccccc4c4c5c(ccc43)oc3ccccc35)c(C#N)c2C#N)c1 ; N#Cc1ccc(-c2ccc(-n3c4ccccc4c4c5sc6ccccc6c5ccc43)c(C#N)c2C#N)c(-n2c3ccccc3c3c4sc5ccccc5c4ccc32)c1C#N]

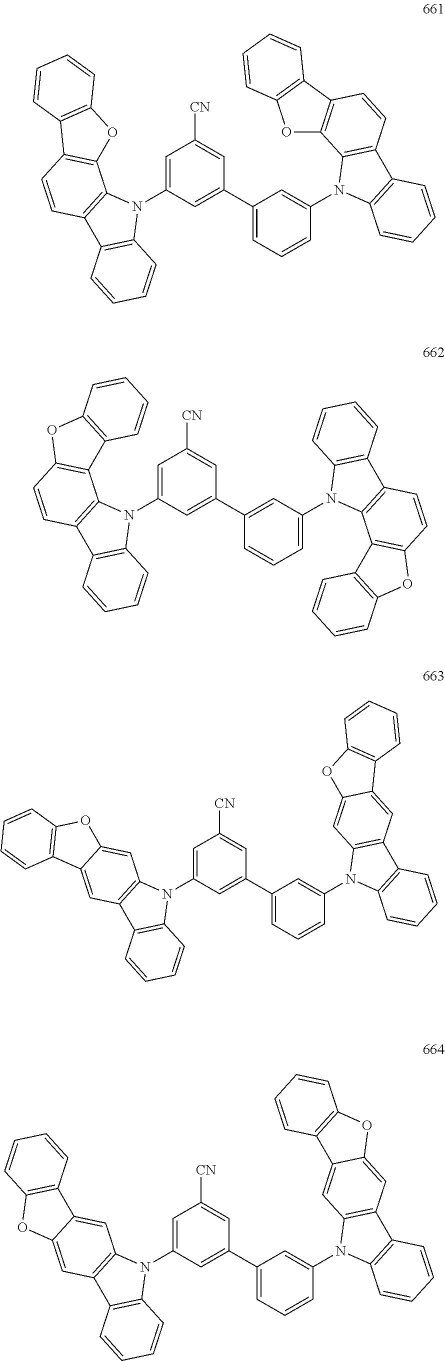 [SMILES: N#Cc1cc(-c2cccc(-n3c4ccccc4c4ccc5c6ccccc6oc5c43)c2)cc(-n2c3ccccc3c3ccc4c5ccccc5oc4c32)c1 ; N#Cc1cc(-c2cccc(-n3c4ccccc4c4ccc5oc6ccccc6c5c43)c2)cc(-n2c3ccccc3c3ccc4oc5ccccc5c4c32)c1 ; N#Cc1cc(-c2cccc(-n3c4ccccc4c4cc5c(cc43)oc3ccccc35)c2)cc(-n2c3ccccc3c3cc4c(cc32)oc2ccccc24)c1 ; N#Cc1cc(-c2cccc(-n3c4ccccc4c4cc5oc6ccccc6c5cc43)c2)cc(-n2c3ccccc3c3cc4oc5ccccc5c4cc32)c1]

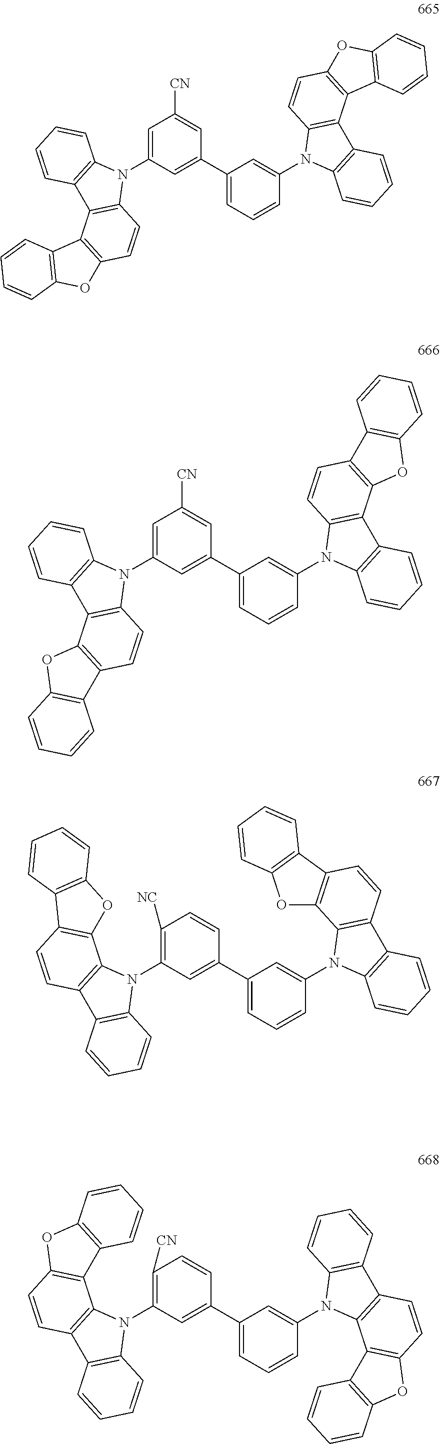 [SMILES: N#Cc1cc(-c2cccc(-n3c4ccccc4c4c5c(ccc43)oc3ccccc35)c2)cc(-n2c3ccccc3c3c4c(ccc32)oc2ccccc24)c1 ; N#Cc1cc(-c2cccc(-n3c4ccccc4c4c5oc6ccccc6c5ccc43)c2)cc(-n2c3ccccc3c3c4oc5ccccc5c4ccc32)c1 ; N#Cc1ccc(-c2cccc(-n3c4ccccc4c4ccc5c6ccccc6oc5c43)c2)cc1-n1c2ccccc2c2ccc3c4ccccc4oc3c21 ; N#Cc1ccc(-c2cccc(-n3c4ccccc4c4ccc5oc6ccccc6c5c43)c2)cc1-n1c2ccccc2c2ccc3oc4ccccc4c3c21]

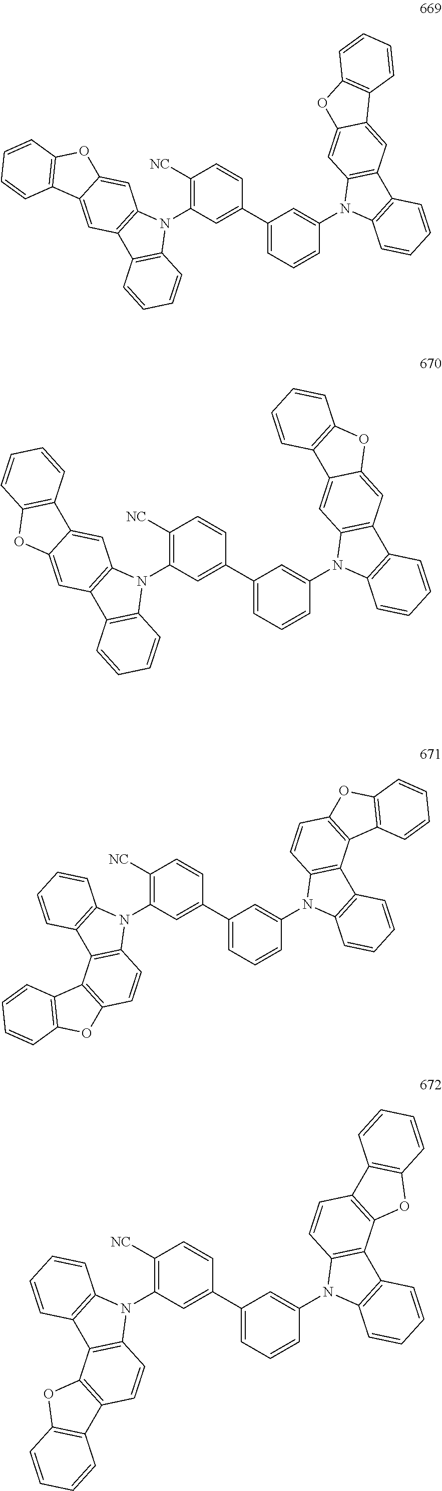 [SMILES: N#Cc1ccc(-c2cccc(-n3c4ccccc4c4cc5c(cc43)oc3ccccc35)c2)cc1-n1c2ccccc2c2cc3c(cc21)oc1ccccc13 ; N#Cc1ccc(-c2cccc(-n3c4ccccc4c4cc5oc6ccccc6c5cc43)c2)cc1-n1c2ccccc2c2cc3oc4ccccc4c3cc21 ; N#Cc1ccc(-c2cccc(-n3c4ccccc4c4c5c(ccc43)oc3ccccc35)c2)cc1-n1c2ccccc2c2c3c(ccc21)oc1ccccc13 ; N#Cc1ccc(-c2cccc(-n3c4ccccc4c4c5oc6ccccc6c5ccc43)c2)cc1-n1c2ccccc2c2c3oc4ccccc4c3ccc21]

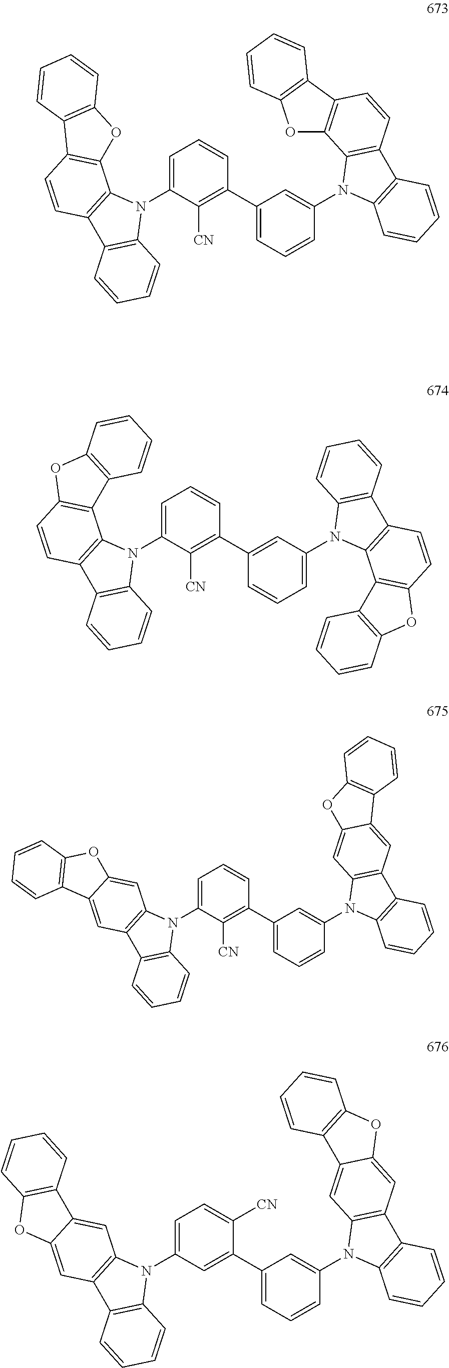 [SMILES: N#Cc1c(-c2cccc(-n3c4ccccc4c4ccc5c6ccccc6oc5c43)c2)cccc1-n1c2ccccc2c2ccc3c4ccccc4oc3c21 ; N#Cc1c(-c2cccc(-n3c4ccccc4c4ccc5oc6ccccc6c5c43)c2)cccc1-n1c2ccccc2c2ccc3oc4ccccc4c3c21 ; N#Cc1c(-c2cccc(-n3c4ccccc4c4cc5c(cc43)oc3ccccc35)c2)cccc1-n1c2ccccc2c2cc3c(cc21)oc1ccccc13 ; N#Cc1ccc(-n2c3ccccc3c3cc4oc5ccccc5c4cc32)cc1-c1cccc(-n2c3ccccc3c3cc4oc5ccccc5c4cc32)c1]

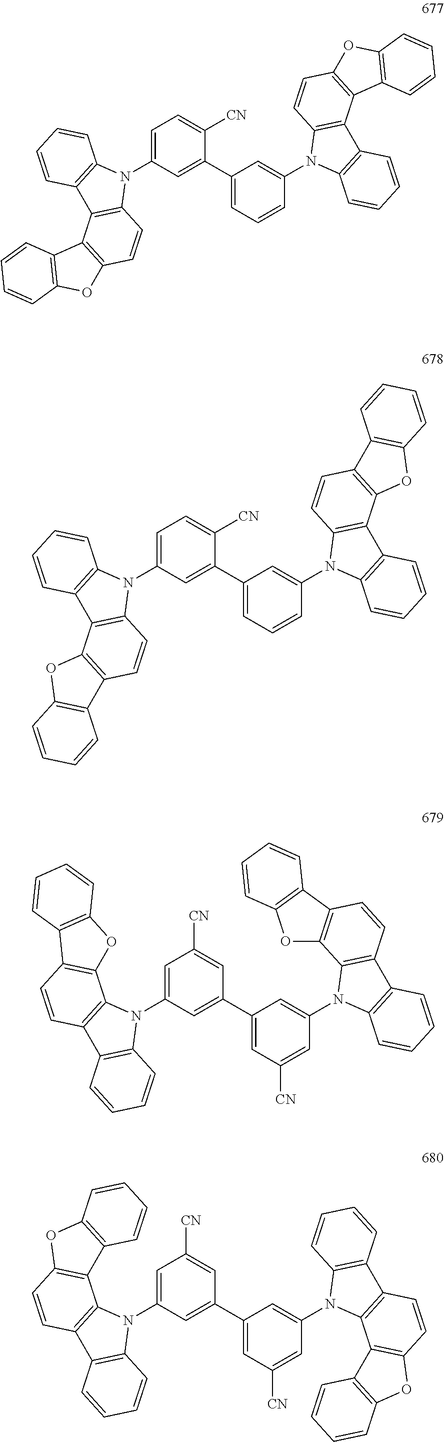 [SMILES: N#Cc1ccc(-n2c3ccccc3c3c4c(ccc32)oc2ccccc24)cc1-c1cccc(-n2c3ccccc3c3c4c(ccc32)oc2ccccc24)c1 ; N#Cc1ccc(-n2c3ccccc3c3c4oc5ccccc5c4ccc32)cc1-c1cccc(-n2c3ccccc3c3c4oc5ccccc5c4ccc32)c1 ; N#Cc1cc(-c2cc(C#N)cc(-n3c4ccccc4c4ccc5c6ccccc6oc5c43)c2)cc(-n2c3ccccc3c3ccc4c5ccccc5oc4c32)c1 ; N#Cc1cc(-c2cc(C#N)cc(-n3c4ccccc4c4ccc5oc6ccccc6c5c43)c2)cc(-n2c3ccccc3c3ccc4oc5ccccc5c4c32)c1]

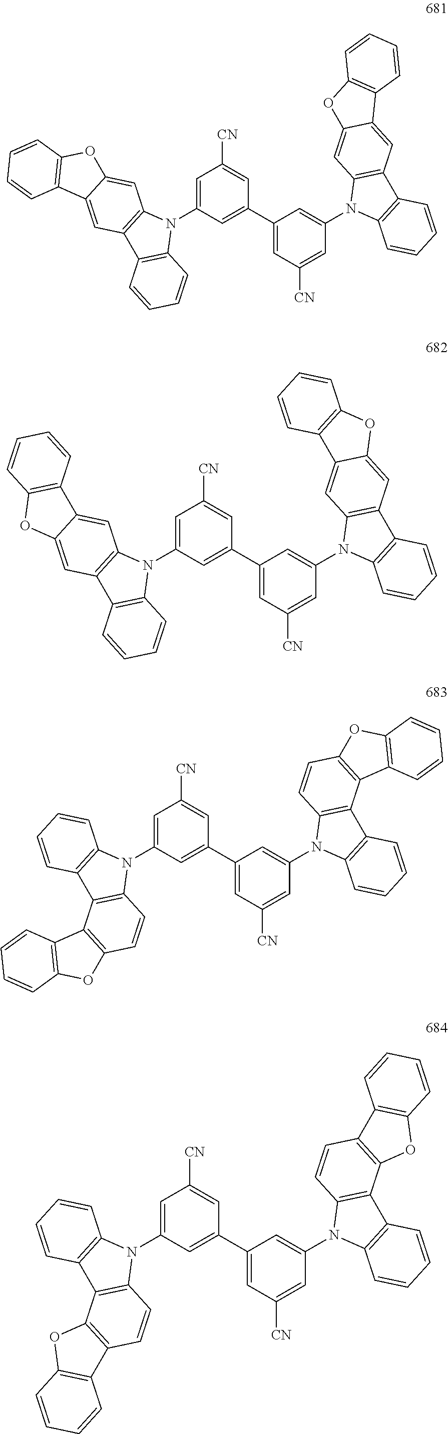 [SMILES: N#Cc1cc(-c2cc(C#N)cc(-n3c4ccccc4c4cc5c(cc43)oc3ccccc35)c2)cc(-n2c3ccccc3c3cc4c(cc32)oc2ccccc24)c1 ; N#Cc1cc(-c2cc(C#N)cc(-n3c4ccccc4c4cc5oc6ccccc6c5cc43)c2)cc(-n2c3ccccc3c3cc4oc5ccccc5c4cc32)c1 ; N#Cc1cc(-c2cc(C#N)cc(-n3c4ccccc4c4c5c(ccc43)oc3ccccc35)c2)cc(-n2c3ccccc3c3c4c(ccc32)oc2ccccc24)c1 ; N#Cc1cc(-c2cc(C#N)cc(-n3c4ccccc4c4c5oc6ccccc6c5ccc43)c2)cc(-n2c3ccccc3c3c4oc5ccccc5c4ccc32)c1]

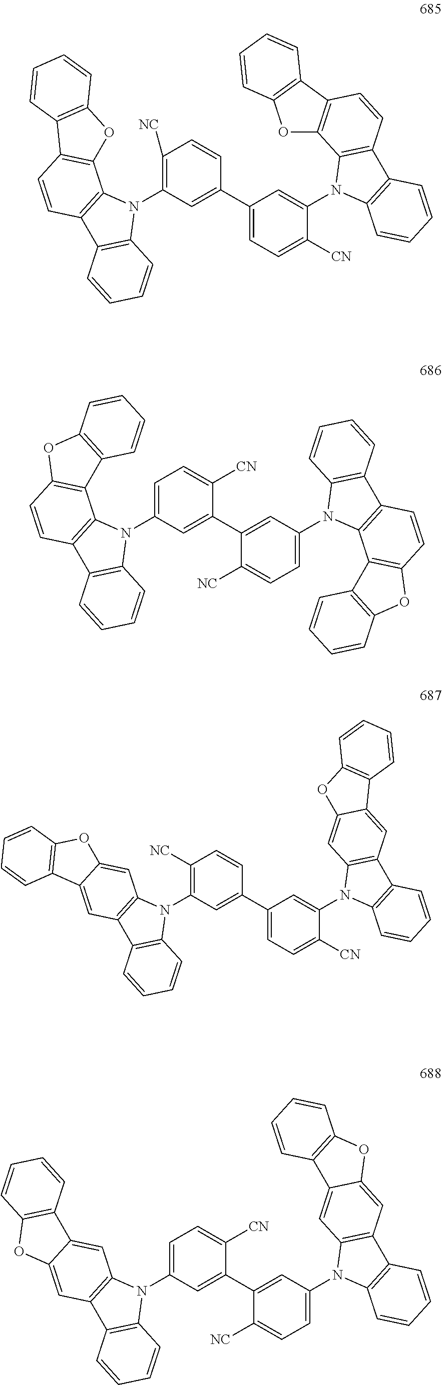 [SMILES: N#Cc1ccc(-c2ccc(C#N)c(-n3c4ccccc4c4ccc5c6ccccc6oc5c43)c2)cc1-n1c2ccccc2c2ccc3c4ccccc4oc3c21 ; N#Cc1ccc(-n2c3ccccc3c3ccc4oc5ccccc5c4c32)cc1-c1cc(-n2c3ccccc3c3ccc4oc5ccccc5c4c32)ccc1C#N ; N#Cc1ccc(-c2ccc(C#N)c(-n3c4ccccc4c4cc5c(cc43)oc3ccccc35)c2)cc1-n1c2ccccc2c2cc3c(cc21)oc1ccccc13 ; N#Cc1ccc(-n2c3ccccc3c3cc4oc5ccccc5c4cc32)cc1-c1cc(-n2c3ccccc3c3cc4oc5ccccc5c4cc32)ccc1C#N]

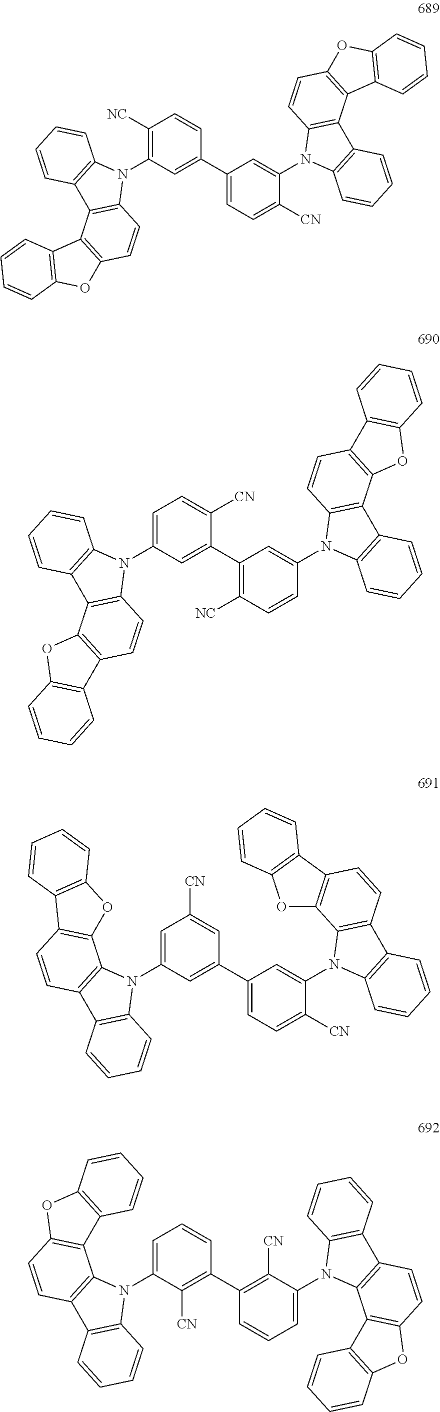 [SMILES: N#Cc1ccc(-c2ccc(C#N)c(-n3c4ccccc4c4c5c(ccc43)oc3ccccc35)c2)cc1-n1c2ccccc2c2c3c(ccc21)oc1ccccc13 ; N#Cc1ccc(-n2c3ccccc3c3c4oc5ccccc5c4ccc32)cc1-c1cc(-n2c3ccccc3c3c4oc5ccccc5c4ccc32)ccc1C#N ; N#Cc1cc(-c2ccc(C#N)c(-n3c4ccccc4c4ccc5c6ccccc6oc5c43)c2)cc(-n2c3ccccc3c3ccc4c5ccccc5oc4c32)c1 ; N#Cc1c(-c2cccc(-n3c4ccccc4c4ccc5oc6ccccc6c5c43)c2C#N)cccc1-n1c2ccccc2c2ccc3oc4ccccc4c3c21]

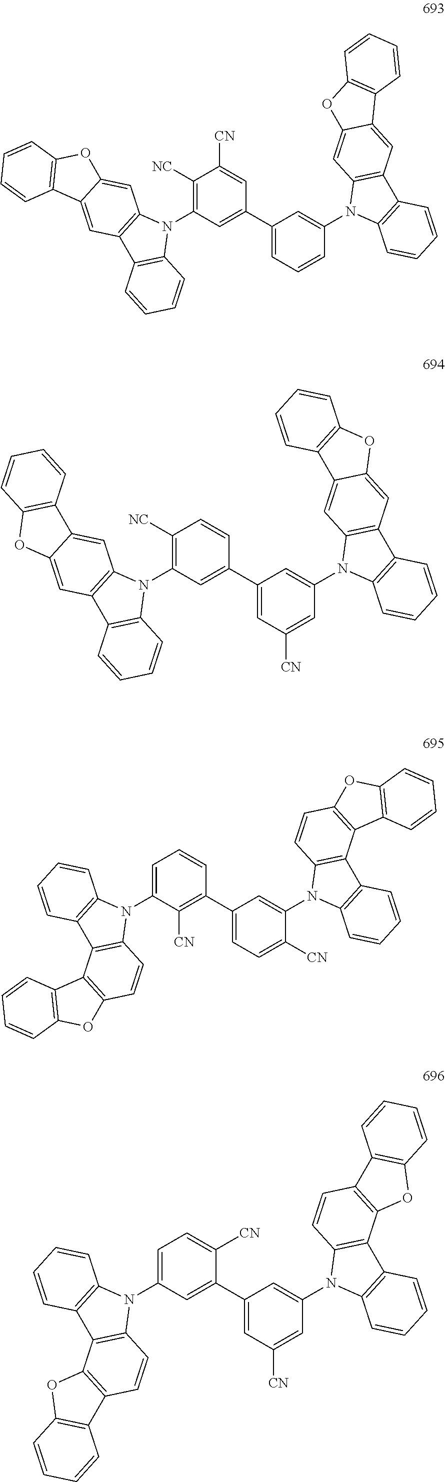 [SMILES: N#Cc1cc(-c2cccc(-n3c4ccccc4c4cc5c(cc43)oc3ccccc35)c2)cc(-n2c3ccccc3c3cc4c(cc32)oc2ccccc24)c1C#N ; N#Cc1cc(-c2ccc(C#N)c(-n3c4ccccc4c4cc5oc6ccccc6c5cc43)c2)cc(-n2c3ccccc3c3cc4oc5ccccc5c4cc32)c1 ; N#Cc1ccc(-c2cccc(-n3c4ccccc4c4c5c(ccc43)oc3ccccc35)c2C#N)cc1-n1c2ccccc2c2c3c(ccc21)oc1ccccc13 ; N#Cc1cc(-c2cc(-n3c4ccccc4c4c5oc6ccccc6c5ccc43)ccc2C#N)cc(-n2c3ccccc3c3c4oc5ccccc5c4ccc32)c1]

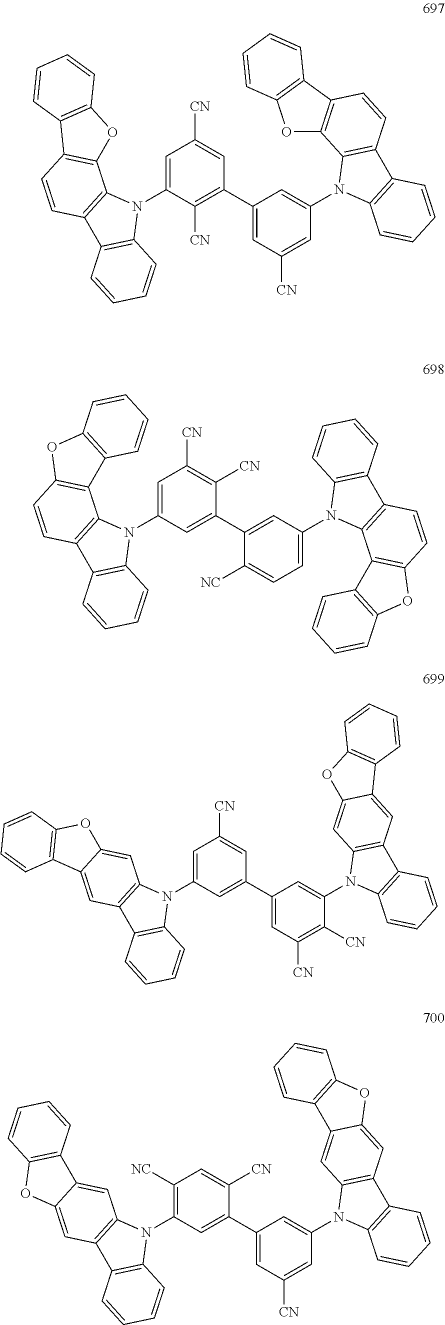 [SMILES: N#Cc1cc(-c2cc(C#N)cc(-n3c4ccccc4c4ccc5c6ccccc6oc5c43)c2C#N)cc(-n2c3ccccc3c3ccc4c5ccccc5oc4c32)c1 ; N#Cc1ccc(-n2c3ccccc3c3ccc4oc5ccccc5c4c32)cc1-c1cc(-n2c3ccccc3c3ccc4oc5ccccc5c4c32)cc(C#N)c1C#N ; N#Cc1cc(-c2cc(C#N)c(C#N)c(-n3c4ccccc4c4cc5c(cc43)oc3ccccc35)c2)cc(-n2c3ccccc3c3cc4c(cc32)oc2ccccc24)c1 ; N#Cc1cc(-c2cc(-n3c4ccccc4c4cc5oc6ccccc6c5cc43)c(C#N)cc2C#N)cc(-n2c3ccccc3c3cc4oc5ccccc5c4cc32)c1]

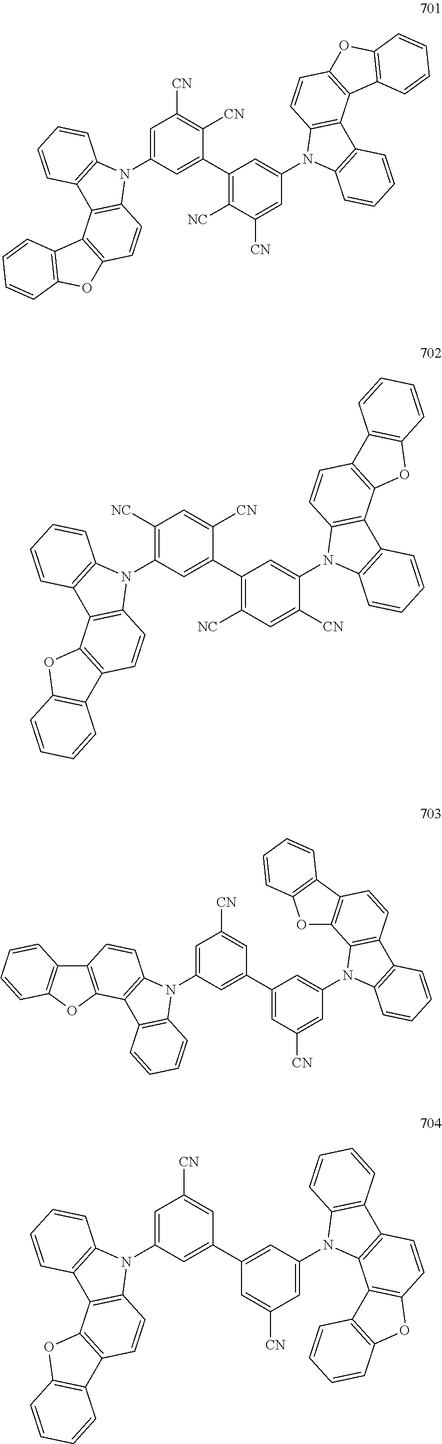 [SMILES: N#Cc1cc(-n2c3ccccc3c3c4c(ccc32)oc2ccccc24)cc(-c2cc(-n3c4ccccc4c4c5c(ccc43)oc3ccccc35)cc(C#N)c2C#N)c1C#N ; N#Cc1cc(C#N)c(-n2c3ccccc3c3c4oc5ccccc5c4ccc32)cc1-c1cc(-n2c3ccccc3c3c4oc5ccccc5c4ccc32)c(C#N)cc1C#N ; N#Cc1cc(-c2cc(C#N)cc(-n3c4ccccc4c4ccc5c6ccccc6oc5c43)c2)cc(-n2c3ccccc3c3c4oc5ccccc5c4ccc32)c1 ; N#Cc1cc(-c2cc(C#N)cc(-n3c4ccccc4c4ccc5oc6ccccc6c5c43)c2)cc(-n2c3ccccc3c3c4oc5ccccc5c4ccc32)c1]

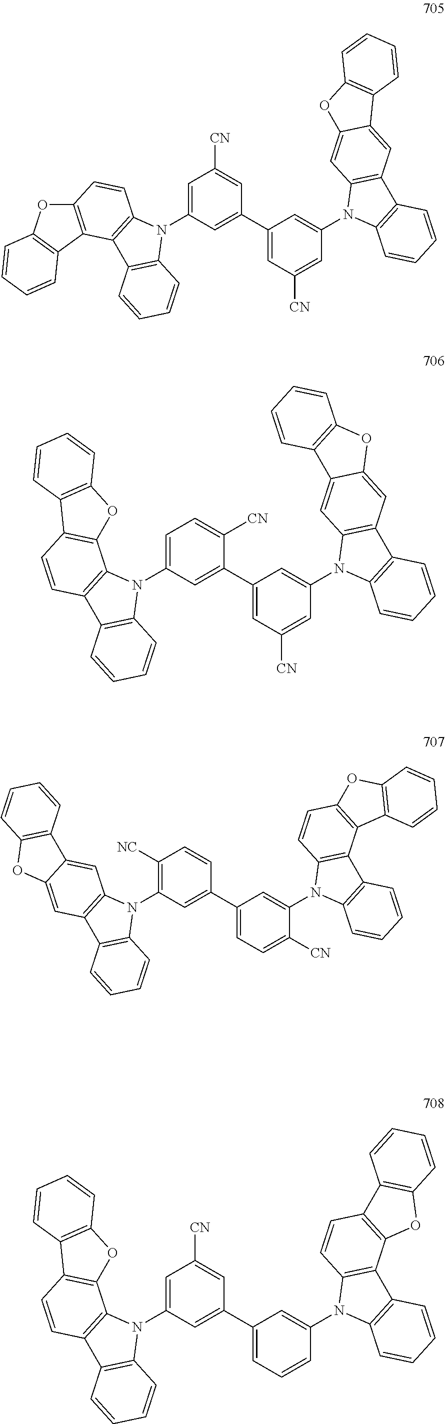 [SMILES: N#Cc1cc(-c2cc(C#N)cc(-n3c4ccccc4c4c5c(ccc43)oc3ccccc35)c2)cc(-n2c3ccccc3c3cc4c(cc32)oc2ccccc24)c1 ; N#Cc1cc(-c2cc(-n3c4ccccc4c4ccc5c6ccccc6oc5c43)ccc2C#N)cc(-n2c3ccccc3c3cc4oc5ccccc5c4cc32)c1 ; N#Cc1ccc(-c2ccc(C#N)c(-n3c4ccccc4c4c5c(ccc43)oc3ccccc35)c2)cc1-n1c2ccccc2c2cc3oc4ccccc4c3cc21 ; N#Cc1cc(-c2cccc(-n3c4ccccc4c4c5oc6ccccc6c5ccc43)c2)cc(-n2c3ccccc3c3ccc4c5ccccc5oc4c32)c1]

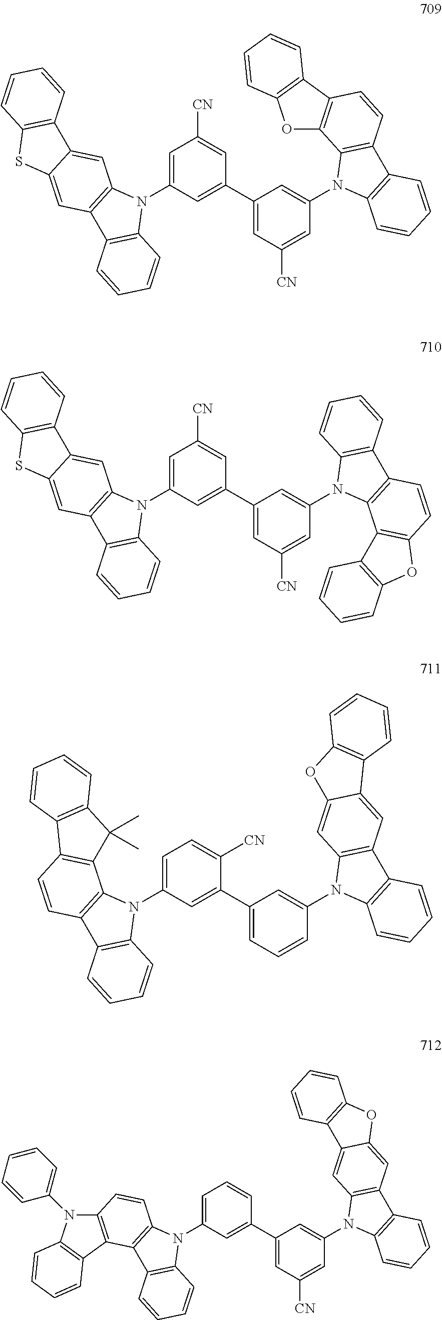 [SMILES: N#Cc1cc(-c2cc(C#N)cc(-n3c4ccccc4c4ccc5c6ccccc6oc5c43)c2)cc(-n2c3ccccc3c3cc4sc5ccccc5c4cc32)c1 ; N#Cc1cc(-c2cc(C#N)cc(-n3c4ccccc4c4ccc5oc6ccccc6c5c43)c2)cc(-n2c3ccccc3c3cc4sc5ccccc5c4cc32)c1 ; CC1(C)c2ccccc2-c2ccc3c4ccccc4n(-c4ccc(C#N)c(-c5cccc(-n6c7ccccc7c7cc8c(cc76)oc6ccccc68)c5)c4)c3c21 ; N#Cc1cc(-c2cccc(-n3c4ccccc4c4c5c6ccccc6n(-c6ccccc6)c5ccc43)c2)cc(-n2c3ccccc3c3cc4oc5ccccc5c4cc32)c1]

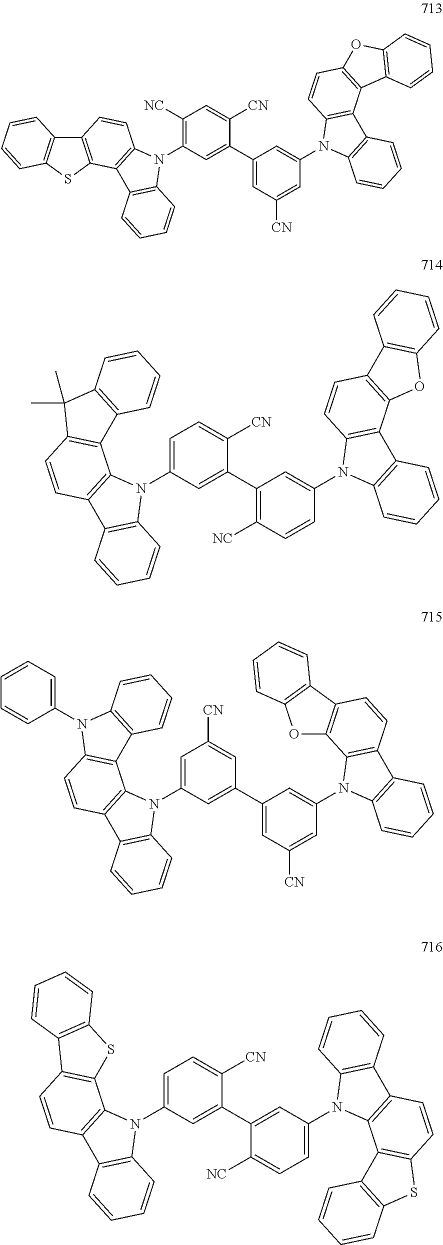 [SMILES: N#Cc1cc(-c2cc(-n3c4ccccc4c4c5sc6ccccc6c5ccc43)c(C#N)cc2C#N)cc(-n2c3ccccc3c3c4c(ccc32)oc2ccccc24)c1 ; CC1(C)c2ccccc2-c2c1ccc1c3ccccc3n(-c3ccc(C#N)c(-c4cc(-n5c6ccccc6c6c7oc8ccccc8c7ccc65)ccc4C#N)c3)c21 ; N#Cc1cc(-c2cc(C#N)cc(-n3c4ccccc4c4ccc5c(c6ccccc6n5-c5ccccc5)c43)c2)cc(-n2c3ccccc3c3ccc4c5ccccc5oc4c32)c1 ; N#Cc1ccc(-n2c3ccccc3c3ccc4c5ccccc5sc4c32)cc1-c1cc(-n2c3ccccc3c3ccc4sc5ccccc5c4c32)ccc1C#N]

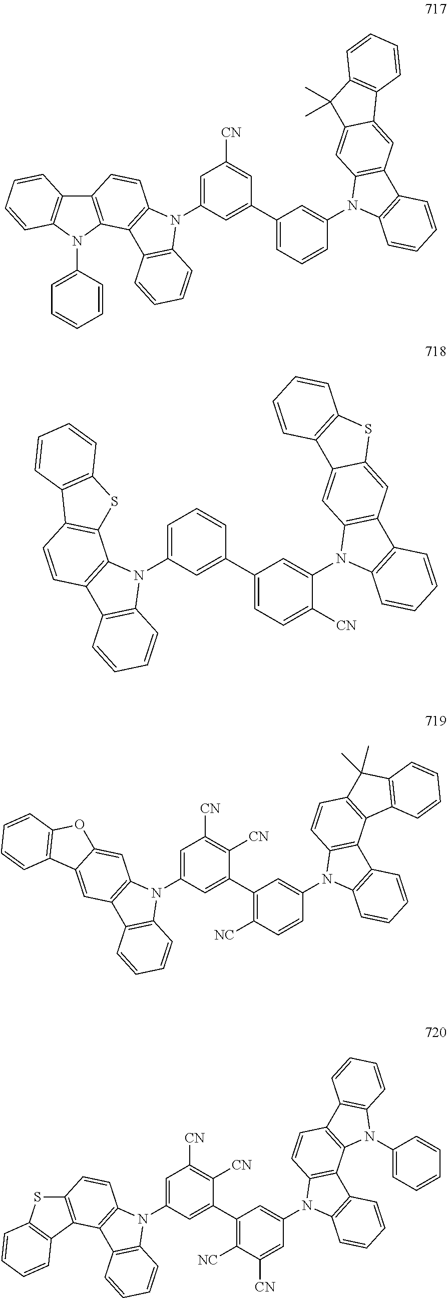 [SMILES: CC1(C)c2ccccc2-c2cc3c4ccccc4n(-c4cccc(-c5cc(C#N)cc(-n6c7ccccc7c7c6ccc6c8ccccc8n(-c8ccccc8)c67)c5)c4)c3cc21 ; N#Cc1ccc(-c2cccc(-n3c4ccccc4c4ccc5c6ccccc6sc5c43)c2)cc1-n1c2ccccc2c2cc3sc4ccccc4c3cc21 ; CC1(C)c2ccccc2-c2c1ccc1c2c2ccccc2n1-c1ccc(C#N)c(-c2cc(-n3c4ccccc4c4cc5c(cc43)oc3ccccc35)cc(C#N)c2C#N)c1 ; N#Cc1cc(-n2c3ccccc3c3c4c(ccc32)sc2ccccc24)cc(-c2cc(-n3c4ccccc4c4c3ccc3c5ccccc5n(-c5ccccc5)c34)cc(C#N)c2C#N)c1C#N]

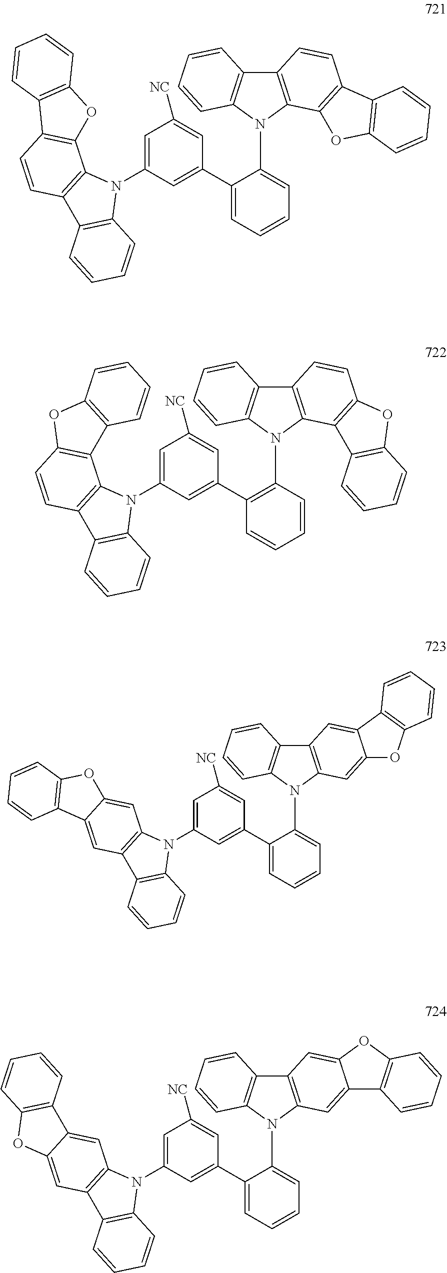 [SMILES: N#Cc1cc(-c2ccccc2-n2c3ccccc3c3ccc4c5ccccc5oc4c32)cc(-n2c3ccccc3c3ccc4c5ccccc5oc4c32)c1 ; N#Cc1cc(-c2ccccc2-n2c3ccccc3c3ccc4oc5ccccc5c4c32)cc(-n2c3ccccc3c3ccc4oc5ccccc5c4c32)c1 ; N#Cc1cc(-c2ccccc2-n2c3ccccc3c3cc4c(cc32)oc2ccccc24)cc(-n2c3ccccc3c3cc4c(cc32)oc2ccccc24)c1 ; N#Cc1cc(-c2ccccc2-n2c3ccccc3c3cc4oc5ccccc5c4cc32)cc(-n2c3ccccc3c3cc4oc5ccccc5c4cc32)c1]

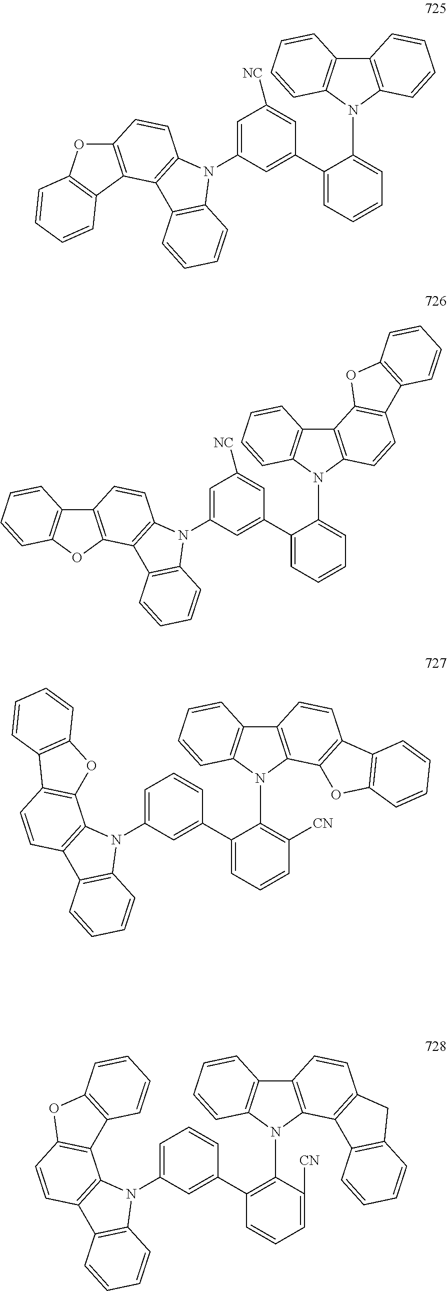 [SMILES: N#Cc1cc(-c2ccccc2-n2c3ccccc3c3ccccc32)cc(-n2c3ccccc3c3c4c(ccc32)oc2ccccc24)c1 ; N#Cc1cc(-c2ccccc2-n2c3ccccc3c3c4oc5ccccc5c4ccc32)cc(-n2c3ccccc3c3c4oc5ccccc5c4ccc32)c1 ; N#Cc1cccc(-c2cccc(-n3c4ccccc4c4ccc5c6ccccc6oc5c43)c2)c1-n1c2ccccc2c2ccc3c4ccccc4oc3c21 ; N#Cc1cccc(-c2cccc(-n3c4ccccc4c4ccc5oc6ccccc6c5c43)c2)c1-n1c2ccccc2c2ccc3c(c21)-c1ccccc1C3]

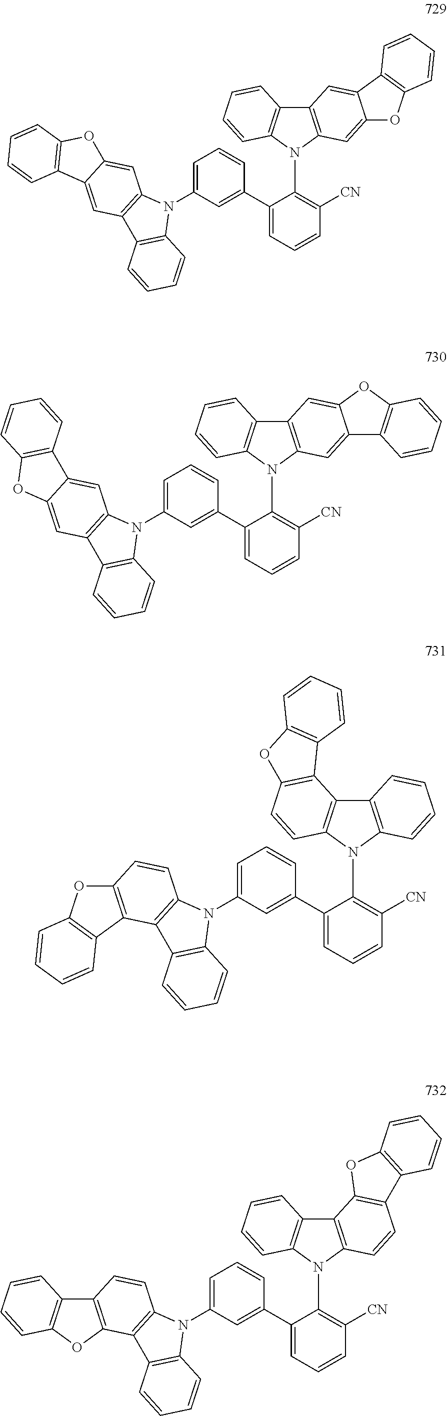 [SMILES: N#Cc1cccc(-c2cccc(-n3c4ccccc4c4cc5c(cc43)oc3ccccc35)c2)c1-n1c2ccccc2c2cc3c(cc21)oc1ccccc13 ; N#Cc1cccc(-c2cccc(-n3c4ccccc4c4cc5oc6ccccc6c5cc43)c2)c1-n1c2ccccc2c2cc3oc4ccccc4c3cc21 ; N#Cc1cccc(-c2cccc(-n3c4ccccc4c4c5c(ccc43)oc3ccccc35)c2)c1-n1c2ccccc2c2c3c(ccc21)oc1ccccc13 ; N#Cc1cccc(-c2cccc(-n3c4ccccc4c4c5oc6ccccc6c5ccc43)c2)c1-n1c2ccccc2c2c3oc4ccccc4c3ccc21]

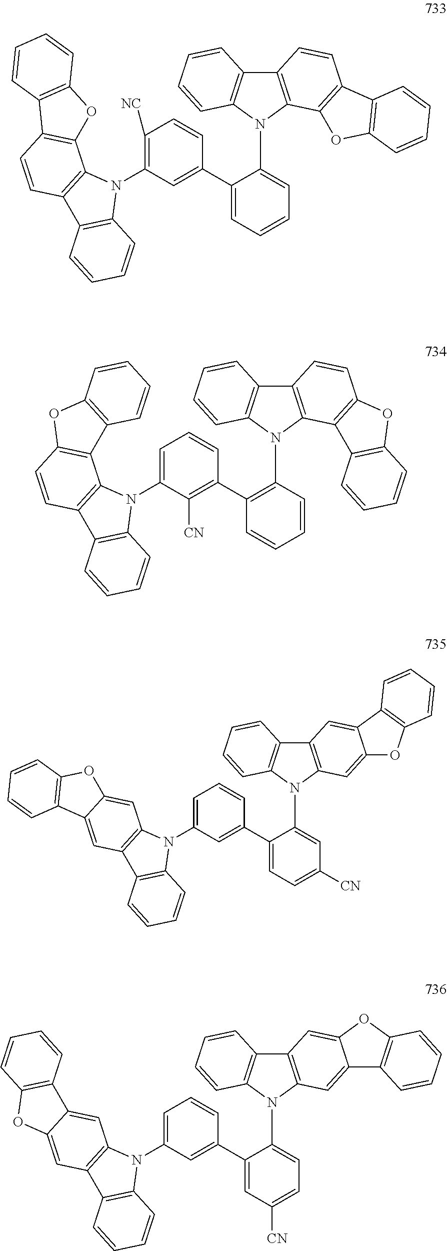 [SMILES: N#Cc1ccc(-c2ccccc2-n2c3ccccc3c3ccc4c5ccccc5oc4c32)cc1-n1c2ccccc2c2ccc3c4ccccc4oc3c21 ; N#Cc1c(-c2ccccc2-n2c3ccccc3c3ccc4oc5ccccc5c4c32)cccc1-n1c2ccccc2c2ccc3oc4ccccc4c3c21 ; N#Cc1ccc(-c2cccc(-n3c4ccccc4c4cc5c(cc43)oc3ccccc35)c2)c(-n2c3ccccc3c3cc4c(cc32)oc2ccccc24)c1 ; N#Cc1ccc(-n2c3ccccc3c3cc4oc5ccccc5c4cc32)c(-c2cccc(-n3c4ccccc4c4cc5oc6ccccc6c5cc43)c2)c1]

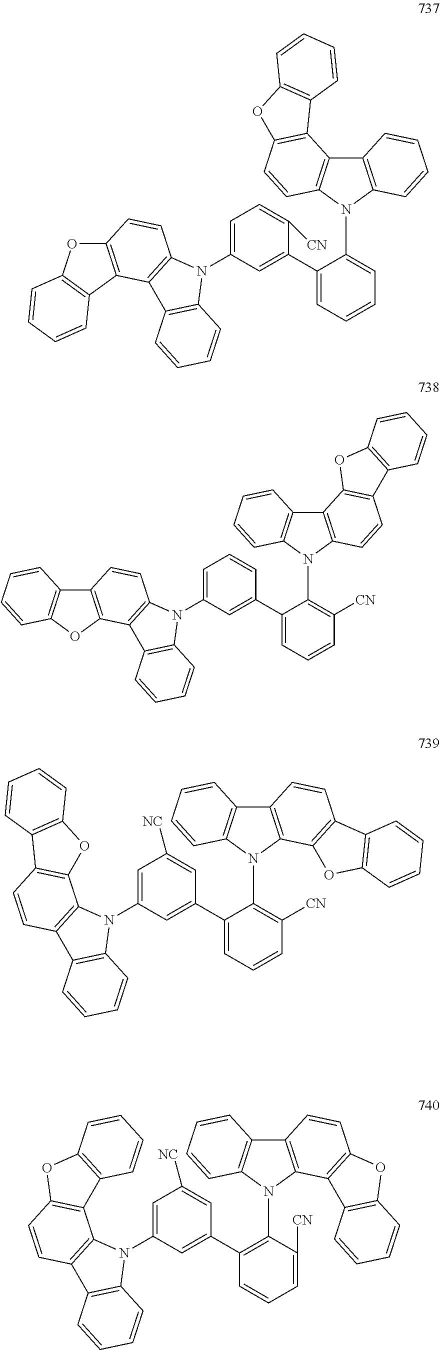 [SMILES: N#Cc1ccc(-n2c3ccccc3c3c4c(ccc32)oc2ccccc24)cc1-c1ccccc1-n1c2ccccc2c2c3c(ccc21)oc1ccccc13 ; N#Cc1cccc(-c2cccc(-n3c4ccccc4c4c5oc6ccccc6c5ccc43)c2)c1-n1c2ccccc2c2c3oc4ccccc4c3ccc21 ; N#Cc1cc(-c2cccc(C#N)c2-n2c3ccccc3c3ccc4c5ccccc5oc4c32)cc(-n2c3ccccc3c3ccc4c5ccccc5oc4c32)c1 ; N#Cc1cc(-c2cccc(C#N)c2-n2c3ccccc3c3ccc4oc5ccccc5c4c32)cc(-n2c3ccccc3c3ccc4oc5ccccc5c4c32)c1]

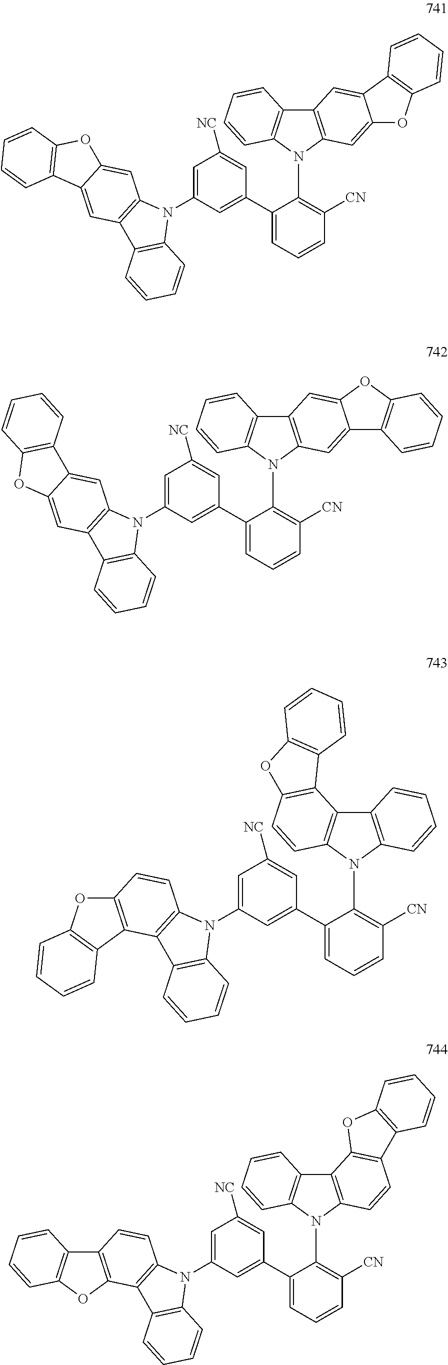 [SMILES: N#Cc1cc(-c2cccc(C#N)c2-n2c3ccccc3c3cc4c(cc32)oc2ccccc24)cc(-n2c3ccccc3c3cc4c(cc32)oc2ccccc24)c1 ; N#Cc1cc(-c2cccc(C#N)c2-n2c3ccccc3c3cc4oc5ccccc5c4cc32)cc(-n2c3ccccc3c3cc4oc5ccccc5c4cc32)c1 ; N#Cc1cc(-c2cccc(C#N)c2-n2c3ccccc3c3c4c(ccc32)oc2ccccc24)cc(-n2c3ccccc3c3c4c(ccc32)oc2ccccc24)c1 ; N#Cc1cc(-c2cccc(C#N)c2-n2c3ccccc3c3c4oc5ccccc5c4ccc32)cc(-n2c3ccccc3c3c4oc5ccccc5c4ccc32)c1]

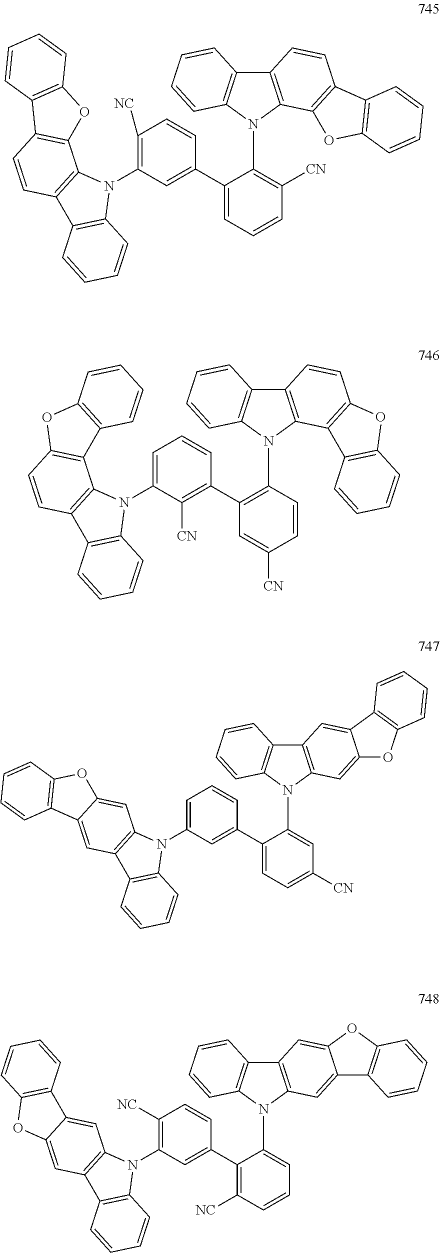 [SMILES: N#Cc1ccc(-c2cccc(C#N)c2-n2c3ccccc3c3ccc4c5ccccc5oc4c32)cc1-n1c2ccccc2c2ccc3c4ccccc4oc3c21 ; N#Cc1ccc(-n2c3ccccc3c3ccc4oc5ccccc5c4c32)c(-c2cccc(-n3c4ccccc4c4ccc5oc6ccccc6c5c43)c2C#N)c1 ; N#Cc1ccc(-c2cccc(-n3c4ccccc4c4cc5c(cc43)oc3ccccc35)c2)c(-n2c3ccccc3c3cc4c(cc32)oc2ccccc24)c1 ; N#Cc1ccc(-c2c(C#N)cccc2-n2c3ccccc3c3cc4oc5ccccc5c4cc32)cc1-n1c2ccccc2c2cc3oc4ccccc4c3cc21]

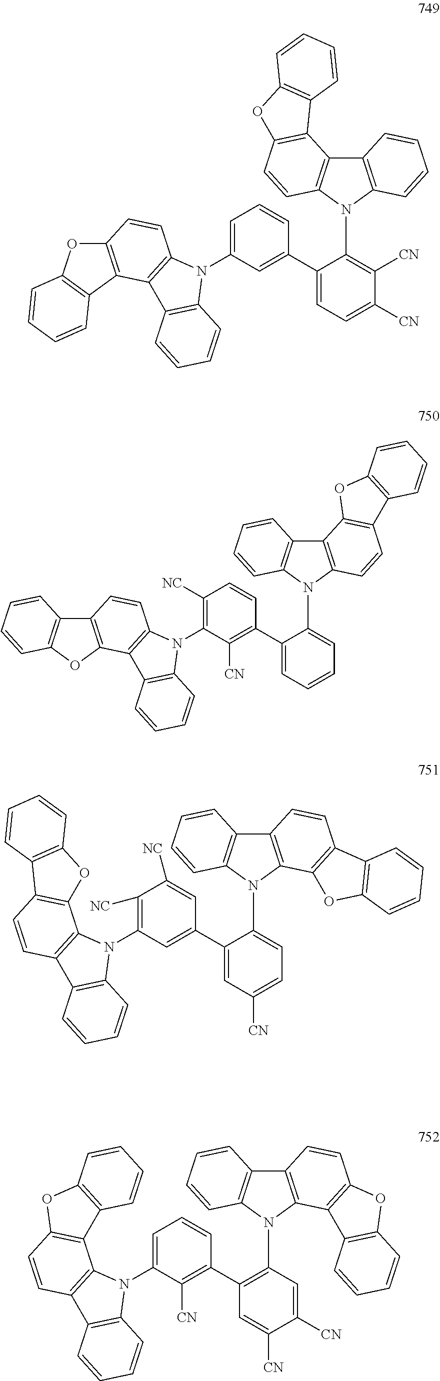 [SMILES: N#Cc1ccc(-c2cccc(-n3c4ccccc4c4c5c(ccc43)oc3ccccc35)c2)c(-n2c3ccccc3c3c4c(ccc32)oc2ccccc24)c1C#N ; N#Cc1ccc(-c2ccccc2-n2c3ccccc3c3c4oc5ccccc5c4ccc32)c(C#N)c1-n1c2ccccc2c2c3oc4ccccc4c3ccc21 ; N#Cc1ccc(-n2c3ccccc3c3ccc4c5ccccc5oc4c32)c(-c2cc(C#N)c(C#N)c(-n3c4ccccc4c4ccc5c6ccccc6oc5c43)c2)c1 ; N#Cc1cc(-c2cccc(-n3c4ccccc4c4ccc5oc6ccccc6c5c43)c2C#N)c(-n2c3ccccc3c3ccc4oc5ccccc5c4c32)cc1C#N]

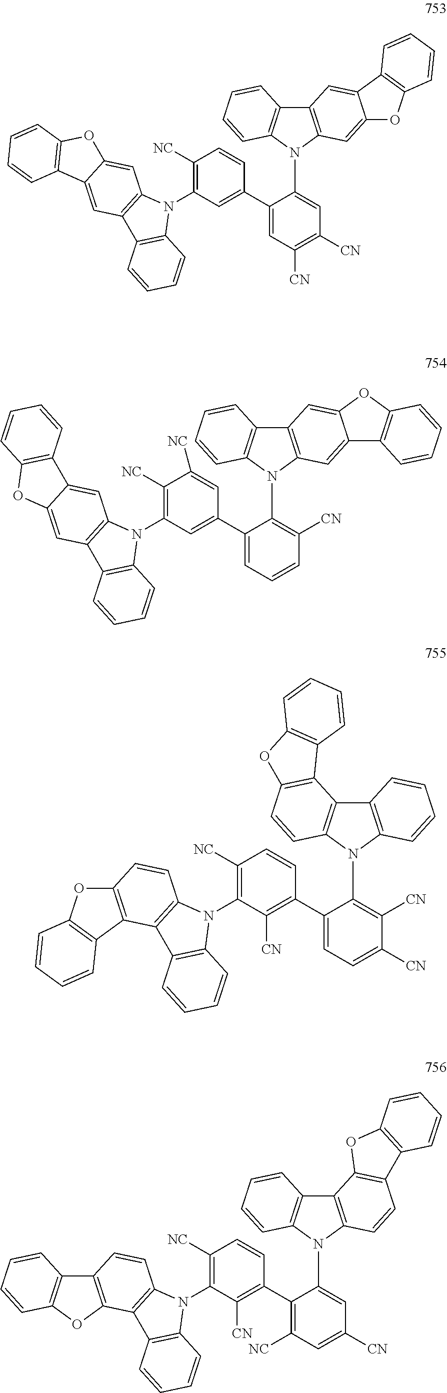 [SMILES: N#Cc1cc(-c2ccc(C#N)c(-n3c4ccccc4c4cc5c(cc43)oc3ccccc35)c2)c(-n2c3ccccc3c3cc4c(cc32)oc2ccccc24)cc1C#N ; N#Cc1cc(-c2cccc(C#N)c2-n2c3ccccc3c3cc4oc5ccccc5c4cc32)cc(-n2c3ccccc3c3cc4oc5ccccc5c4cc32)c1C#N ; N#Cc1ccc(-c2ccc(C#N)c(-n3c4ccccc4c4c5c(ccc43)oc3ccccc35)c2C#N)c(-n2c3ccccc3c3c4c(ccc32)oc2ccccc24)c1C#N ; N#Cc1cc(C#N)c(-c2ccc(C#N)c(-n3c4ccccc4c4c5oc6ccccc6c5ccc43)c2C#N)c(-n2c3ccccc3c3c4oc5ccccc5c4ccc32)c1]

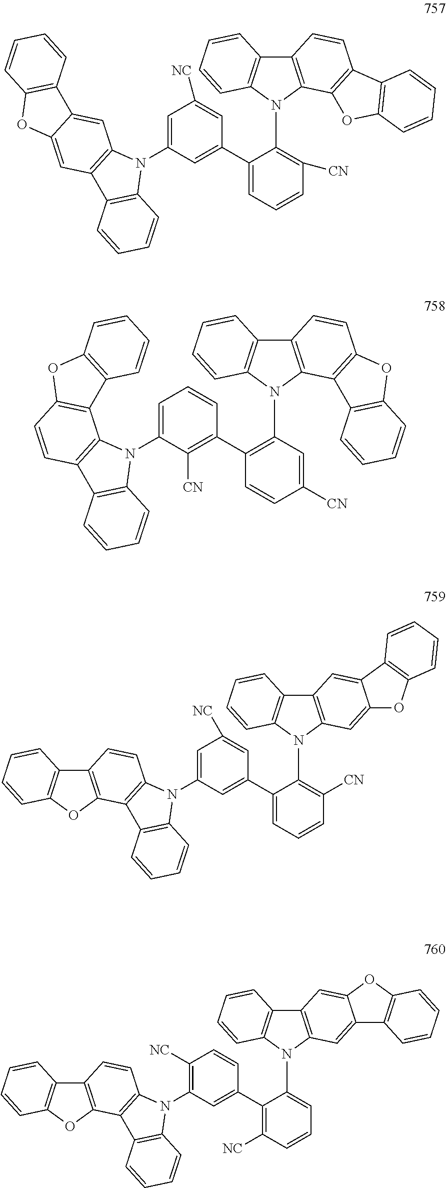 [SMILES: N#Cc1cc(-c2cccc(C#N)c2-n2c3ccccc3c3ccc4c5ccccc5oc4c32)cc(-n2c3ccccc3c3cc4oc5ccccc5c4cc32)c1 ; N#Cc1ccc(-c2cccc(-n3c4ccccc4c4ccc5oc6ccccc6c5c43)c2C#N)c(-n2c3ccccc3c3ccc4oc5ccccc5c4c32)c1 ; N#Cc1cc(-c2cccc(C#N)c2-n2c3ccccc3c3cc4c(cc32)oc2ccccc24)cc(-n2c3ccccc3c3c4oc5ccccc5c4ccc32)c1 ; N#Cc1ccc(-c2c(C#N)cccc2-n2c3ccccc3c3cc4oc5ccccc5c4cc32)cc1-n1c2ccccc2c2c3oc4ccccc4c3ccc21]

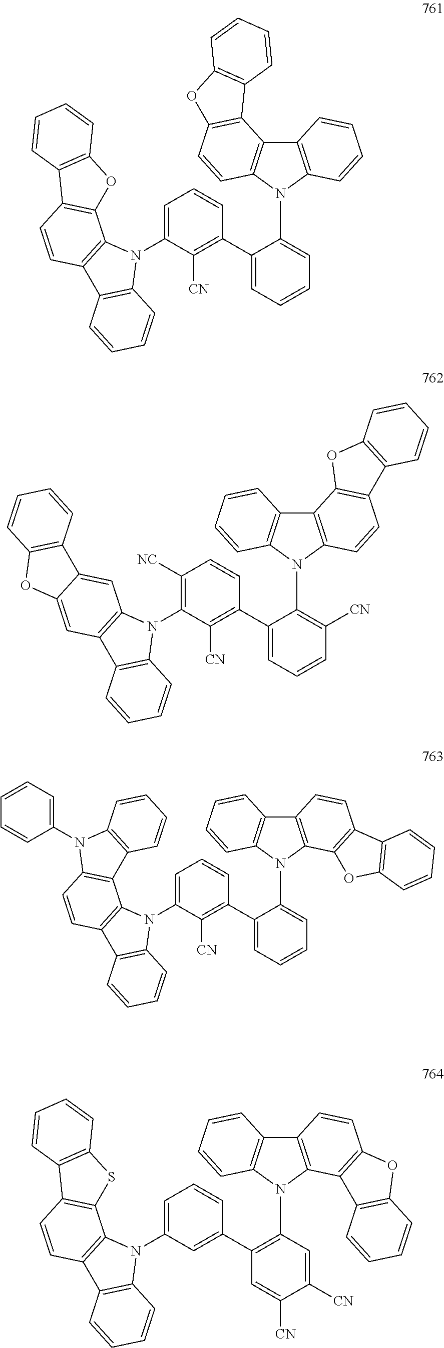 [SMILES: N#Cc1c(-c2ccccc2-n2c3ccccc3c3c4c(ccc32)oc2ccccc24)cccc1-n1c2ccccc2c2ccc3c4ccccc4oc3c21 ; N#Cc1ccc(-c2cccc(C#N)c2-n2c3ccccc3c3c4oc5ccccc5c4ccc32)c(C#N)c1-n1c2ccccc2c2cc3oc4ccccc4c3cc21 ; N#Cc1c(-c2ccccc2-n2c3ccccc3c3ccc4c5ccccc5oc4c32)cccc1-n1c2ccccc2c2ccc3c(c4ccccc4n3-c3ccccc3)c21 ; N#Cc1cc(-c2cccc(-n3c4ccccc4c4ccc5c6ccccc6sc5c43)c2)c(-n2c3ccccc3c3ccc4oc5ccccc5c4c32)cc1C#N]

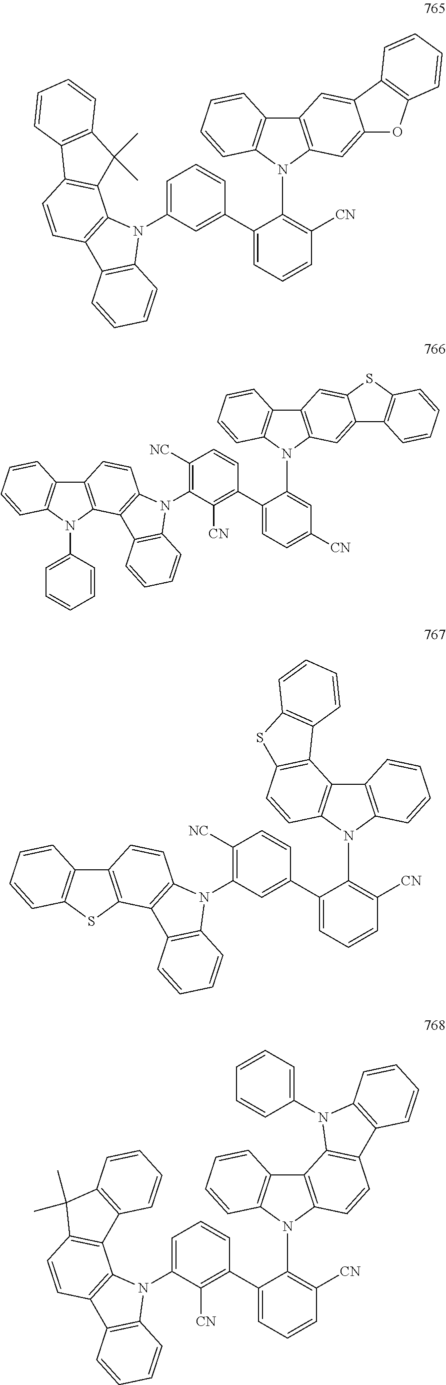 [SMILES: CC1(C)c2ccccc2-c2ccc3c4ccccc4n(-c4cccc(-c5cccc(C#N)c5-n5c6ccccc6c6cc7c(cc65)oc5ccccc57)c4)c3c21 ; N#Cc1ccc(-c2ccc(C#N)c(-n3c4ccccc4c4c3ccc3c5ccccc5n(-c5ccccc5)c34)c2C#N)c(-n2c3ccccc3c3cc4sc5ccccc5c4cc32)c1 ; N#Cc1ccc(-c2cccc(C#N)c2-n2c3ccccc3c3c4c(ccc32)sc2ccccc24)cc1-n1c2ccccc2c2c3sc4ccccc4c3ccc21 ; CC1(C)c2ccccc2-c2c1ccc1c3ccccc3n(-c3cccc(-c4cccc(C#N)c4-n4c5ccccc5c5c4ccc4c6ccccc6n(-c6ccccc6)c45)c3C#N)c21]

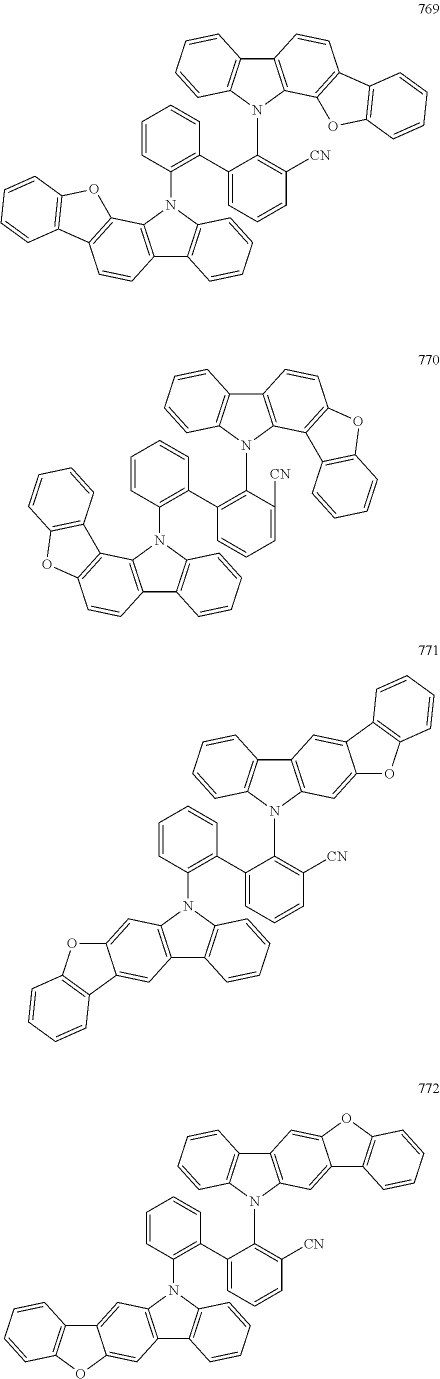 [SMILES: N#Cc1cccc(-c2ccccc2-n2c3ccccc3c3ccc4c5ccccc5oc4c32)c1-n1c2ccccc2c2ccc3c4ccccc4oc3c21 ; N#Cc1cccc(-c2ccccc2-n2c3ccccc3c3ccc4oc5ccccc5c4c32)c1-n1c2ccccc2c2ccc3oc4ccccc4c3c21 ; N#Cc1cccc(-c2ccccc2-n2c3ccccc3c3cc4c(cc32)oc2ccccc24)c1-n1c2ccccc2c2cc3c(cc21)oc1ccccc13 ; N#Cc1cccc(-c2ccccc2-n2c3ccccc3c3cc4oc5ccccc5c4cc32)c1-n1c2ccccc2c2cc3oc4ccccc4c3cc21]

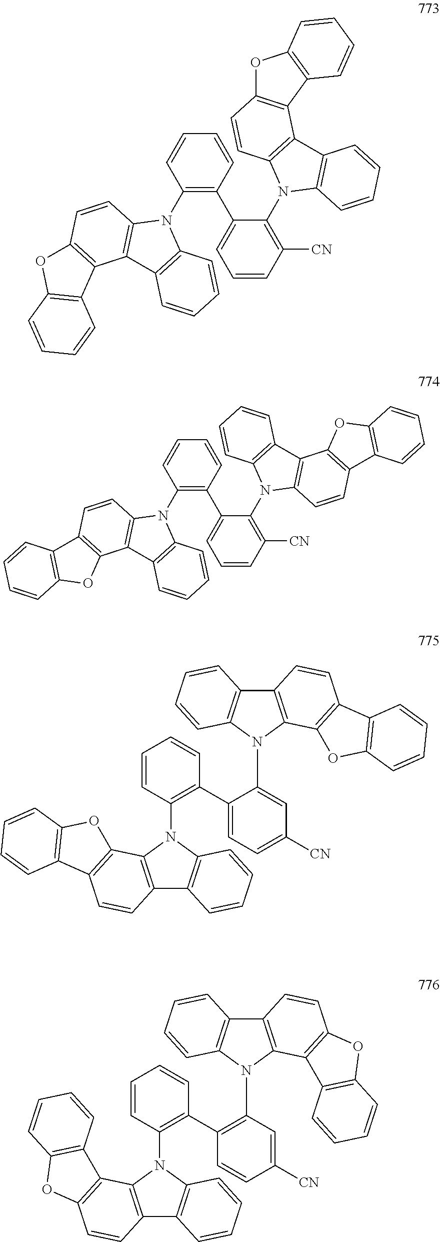 [SMILES: N#Cc1cccc(-c2ccccc2-n2c3ccccc3c3c4c(ccc32)oc2ccccc24)c1-n1c2ccccc2c2c3c(ccc21)oc1ccccc13 ; N#Cc1cccc(-c2ccccc2-n2c3ccccc3c3c4oc5ccccc5c4ccc32)c1-n1c2ccccc2c2c3oc4ccccc4c3ccc21 ; N#Cc1ccc(-c2ccccc2-n2c3ccccc3c3ccc4c5ccccc5oc4c32)c(-n2c3ccccc3c3ccc4c5ccccc5oc4c32)c1 ; N#Cc1ccc(-c2ccccc2-n2c3ccccc3c3ccc4oc5ccccc5c4c32)c(-n2c3ccccc3c3ccc4oc5ccccc5c4c32)c1]

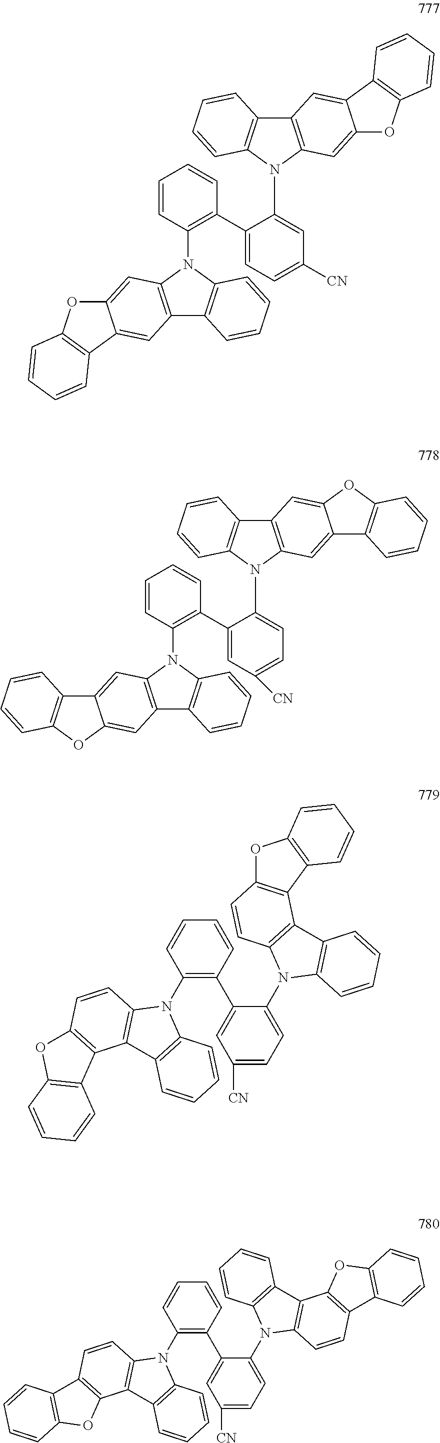 [SMILES: N#Cc1ccc(-c2ccccc2-n2c3ccccc3c3cc4c(cc32)oc2ccccc24)c(-n2c3ccccc3c3cc4c(cc32)oc2ccccc24)c1 ; N#Cc1ccc(-n2c3ccccc3c3cc4oc5ccccc5c4cc32)c(-c2ccccc2-n2c3ccccc3c3cc4oc5ccccc5c4cc32)c1 ; N#Cc1ccc(-n2c3ccccc3c3c4c(ccc32)oc2ccccc24)c(-c2ccccc2-n2c3ccccc3c3c4c(ccc32)oc2ccccc24)c1 ; N#Cc1ccc(-n2c3ccccc3c3c4oc5ccccc5c4ccc32)c(-c2ccccc2-n2c3ccccc3c3c4oc5ccccc5c4ccc32)c1]

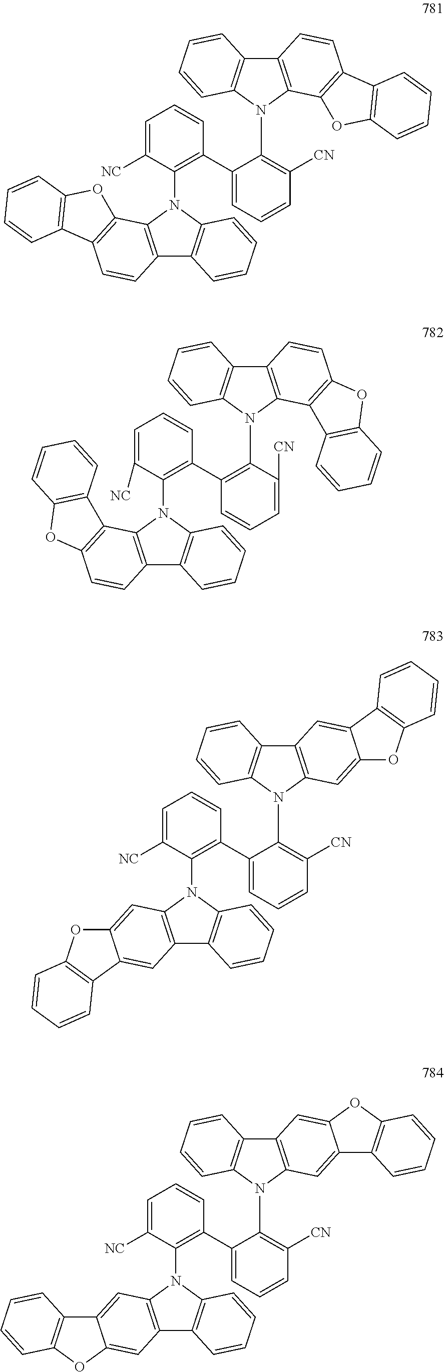 [SMILES: N#Cc1cccc(-c2cccc(C#N)c2-n2c3ccccc3c3ccc4c5ccccc5oc4c32)c1-n1c2ccccc2c2ccc3c4ccccc4oc3c21 ; N#Cc1cccc(-c2cccc(C#N)c2-n2c3ccccc3c3ccc4oc5ccccc5c4c32)c1-n1c2ccccc2c2ccc3oc4ccccc4c3c21 ; N#Cc1cccc(-c2cccc(C#N)c2-n2c3ccccc3c3cc4c(cc32)oc2ccccc24)c1-n1c2ccccc2c2cc3c(cc21)oc1ccccc13 ; N#Cc1cccc(-c2cccc(C#N)c2-n2c3ccccc3c3cc4oc5ccccc5c4cc32)c1-n1c2ccccc2c2cc3oc4ccccc4c3cc21]

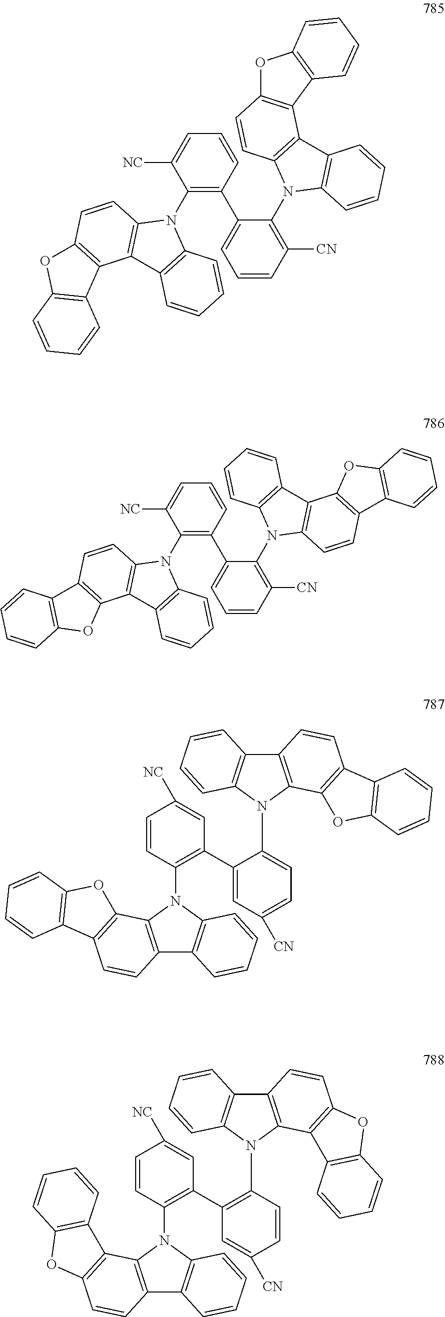 [SMILES: N#Cc1cccc(-c2cccc(C#N)c2-n2c3ccccc3c3c4c(ccc32)oc2ccccc24)c1-n1c2ccccc2c2c3c(ccc21)oc1ccccc13 ; N#Cc1cccc(-c2cccc(C#N)c2-n2c3ccccc3c3c4oc5ccccc5c4ccc32)c1-n1c2ccccc2c2c3oc4ccccc4c3ccc21 ; N#Cc1ccc(-n2c3ccccc3c3ccc4c5ccccc5oc4c32)c(-c2cc(C#N)ccc2-n2c3ccccc3c3ccc4c5ccccc5oc4c32)c1 ; N#Cc1ccc(-n2c3ccccc3c3ccc4oc5ccccc5c4c32)c(-c2cc(C#N)ccc2-n2c3ccccc3c3ccc4oc5ccccc5c4c32)c1]

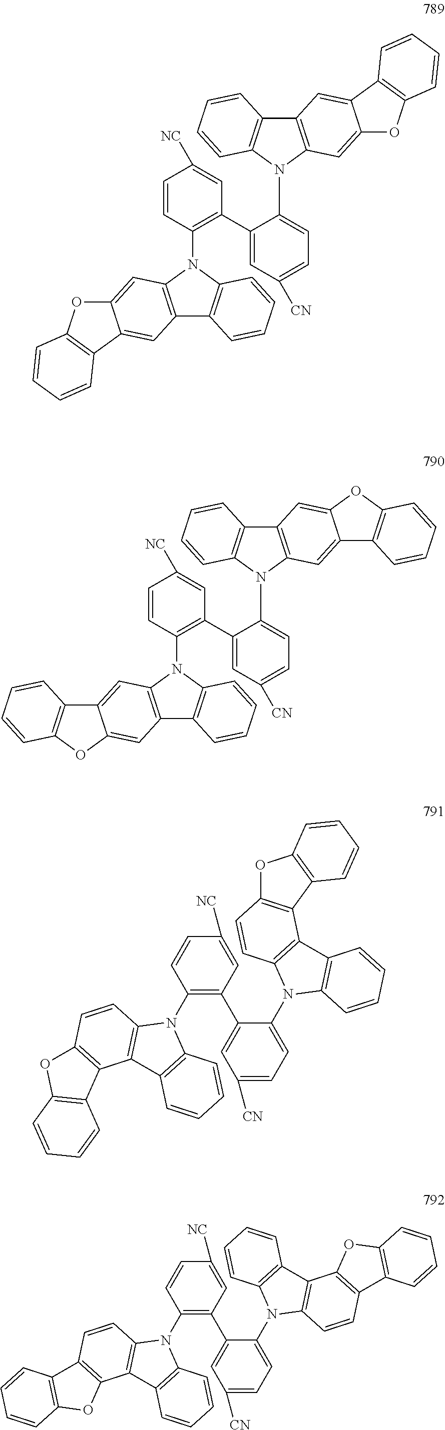 [SMILES: N#Cc1ccc(-n2c3ccccc3c3cc4c(cc32)oc2ccccc24)c(-c2cc(C#N)ccc2-n2c3ccccc3c3cc4c(cc32)oc2ccccc24)c1 ; N#Cc1ccc(-n2c3ccccc3c3cc4oc5ccccc5c4cc32)c(-c2cc(C#N)ccc2-n2c3ccccc3c3cc4oc5ccccc5c4cc32)c1 ; N#Cc1ccc(-n2c3ccccc3c3c4c(ccc32)oc2ccccc24)c(-c2cc(C#N)ccc2-n2c3ccccc3c3c4c(ccc32)oc2ccccc24)c1 ; N#Cc1ccc(-n2c3ccccc3c3c4oc5ccccc5c4ccc32)c(-c2cc(C#N)ccc2-n2c3ccccc3c3c4oc5ccccc5c4ccc32)c1]

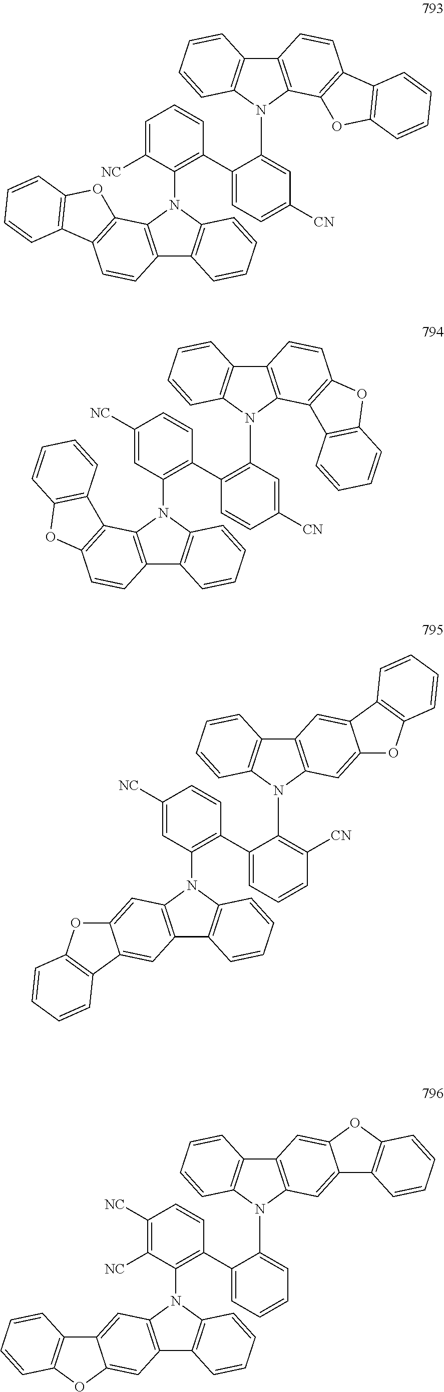 [SMILES: N#Cc1ccc(-c2cccc(C#N)c2-n2c3ccccc3c3ccc4c5ccccc5oc4c32)c(-n2c3ccccc3c3ccc4c5ccccc5oc4c32)c1 ; N#Cc1ccc(-c2ccc(C#N)cc2-n2c3ccccc3c3ccc4oc5ccccc5c4c32)c(-n2c3ccccc3c3ccc4oc5ccccc5c4c32)c1 ; N#Cc1ccc(-c2cccc(C#N)c2-n2c3ccccc3c3cc4c(cc32)oc2ccccc24)c(-n2c3ccccc3c3cc4c(cc32)oc2ccccc24)c1 ; N#Cc1ccc(-c2ccccc2-n2c3ccccc3c3cc4oc5ccccc5c4cc32)c(-n2c3ccccc3c3cc4oc5ccccc5c4cc32)c1C#N]

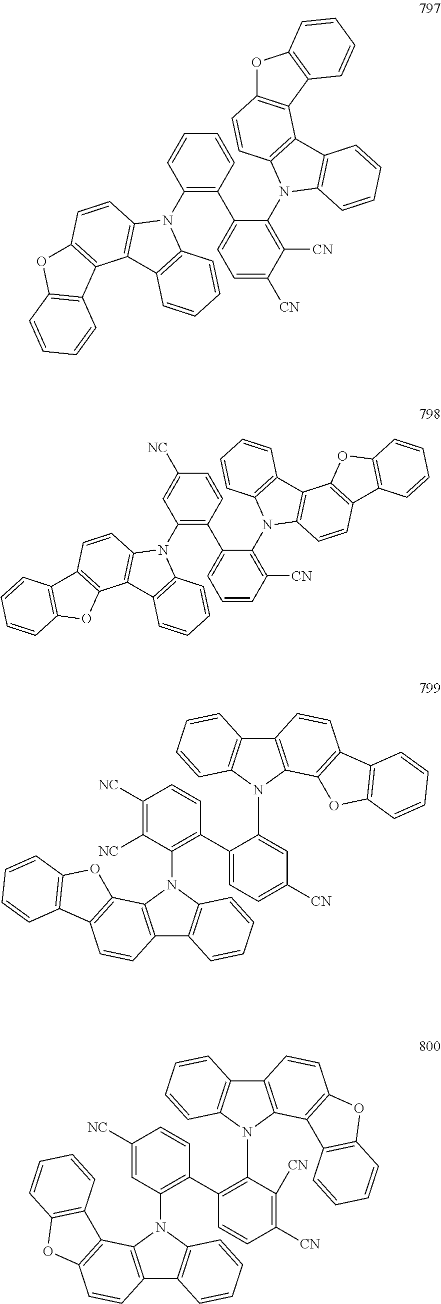 [SMILES: N#Cc1ccc(-c2ccccc2-n2c3ccccc3c3c4c(ccc32)oc2ccccc24)c(-n2c3ccccc3c3c4c(ccc32)oc2ccccc24)c1C#N ; N#Cc1ccc(-c2cccc(C#N)c2-n2c3ccccc3c3c4oc5ccccc5c4ccc32)c(-n2c3ccccc3c3c4oc5ccccc5c4ccc32)c1 ; N#Cc1ccc(-c2ccc(C#N)c(C#N)c2-n2c3ccccc3c3ccc4c5ccccc5oc4c32)c(-n2c3ccccc3c3ccc4c5ccccc5oc4c32)c1 ; N#Cc1ccc(-c2ccc(C#N)c(C#N)c2-n2c3ccccc3c3ccc4oc5ccccc5c4c32)c(-n2c3ccccc3c3ccc4oc5ccccc5c4c32)c1]

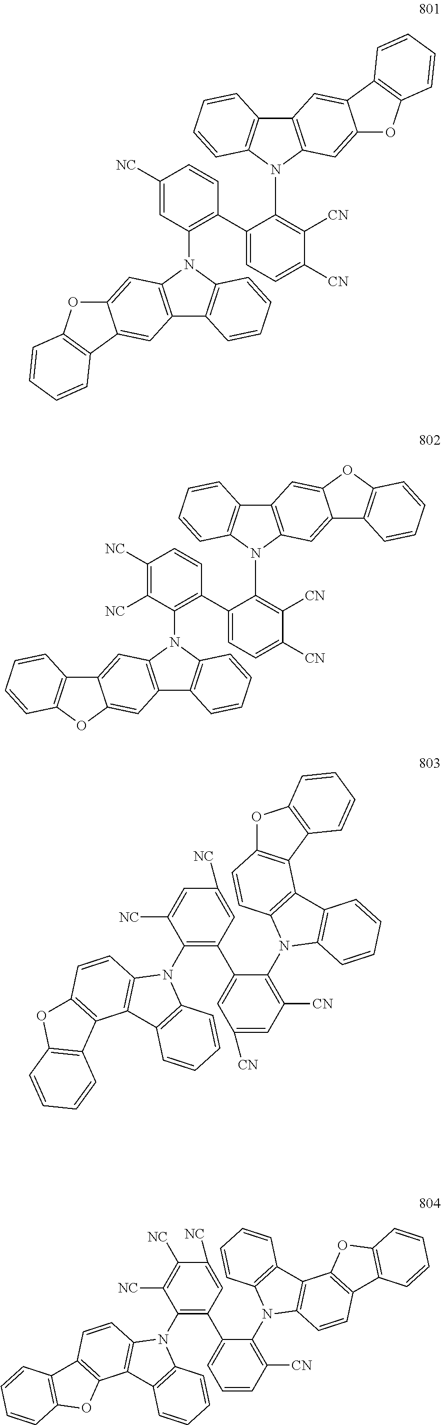 [SMILES: N#Cc1ccc(-c2ccc(C#N)c(C#N)c2-n2c3ccccc3c3cc4c(cc32)oc2ccccc24)c(-n2c3ccccc3c3cc4c(cc32)oc2ccccc24)c1 ; N#Cc1ccc(-c2ccc(C#N)c(C#N)c2-n2c3ccccc3c3cc4oc5ccccc5c4cc32)c(-n2c3ccccc3c3cc4oc5ccccc5c4cc32)c1C#N ; N#Cc1cc(C#N)c(-n2c3ccccc3c3c4c(ccc32)oc2ccccc24)c(-c2cc(C#N)cc(C#N)c2-n2c3ccccc3c3c4c(ccc32)oc2ccccc24)c1 ; N#Cc1cc(-c2cccc(C#N)c2-n2c3ccccc3c3c4oc5ccccc5c4ccc32)c(-n2c3ccccc3c3c4oc5ccccc5c4ccc32)c(C#N)c1C#N]

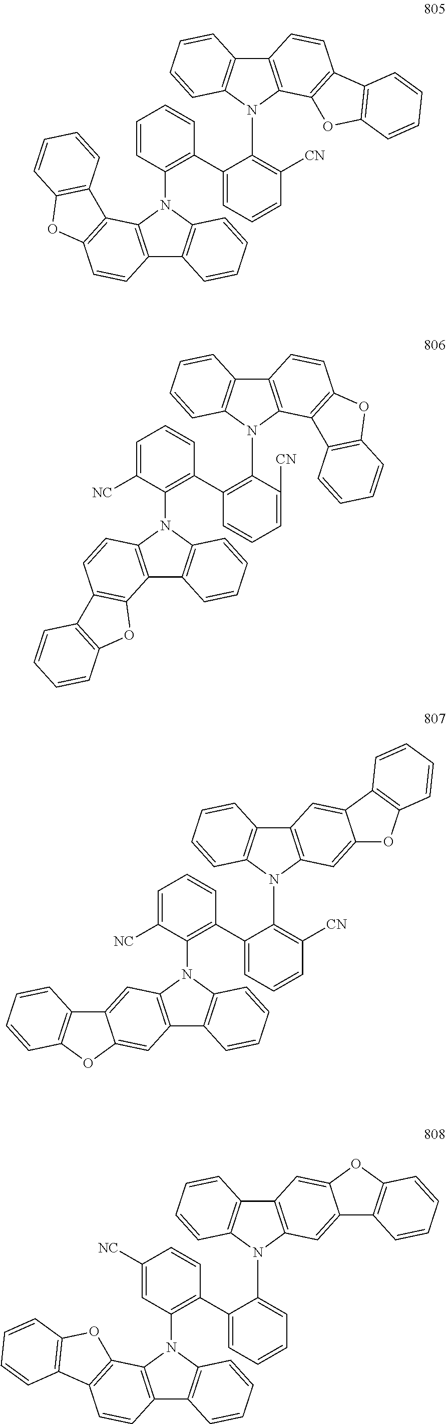 [SMILES: N#Cc1cccc(-c2ccccc2-n2c3ccccc3c3ccc4oc5ccccc5c4c32)c1-n1c2ccccc2c2ccc3c4ccccc4oc3c21 ; N#Cc1cccc(-c2cccc(C#N)c2-n2c3ccccc3c3ccc4oc5ccccc5c4c32)c1-n1c2ccccc2c2c3oc4ccccc4c3ccc21 ; N#Cc1cccc(-c2cccc(C#N)c2-n2c3ccccc3c3cc4oc5ccccc5c4cc32)c1-n1c2ccccc2c2cc3c(cc21)oc1ccccc13 ; N#Cc1ccc(-c2ccccc2-n2c3ccccc3c3cc4oc5ccccc5c4cc32)c(-n2c3ccccc3c3ccc4c5ccccc5oc4c32)c1]

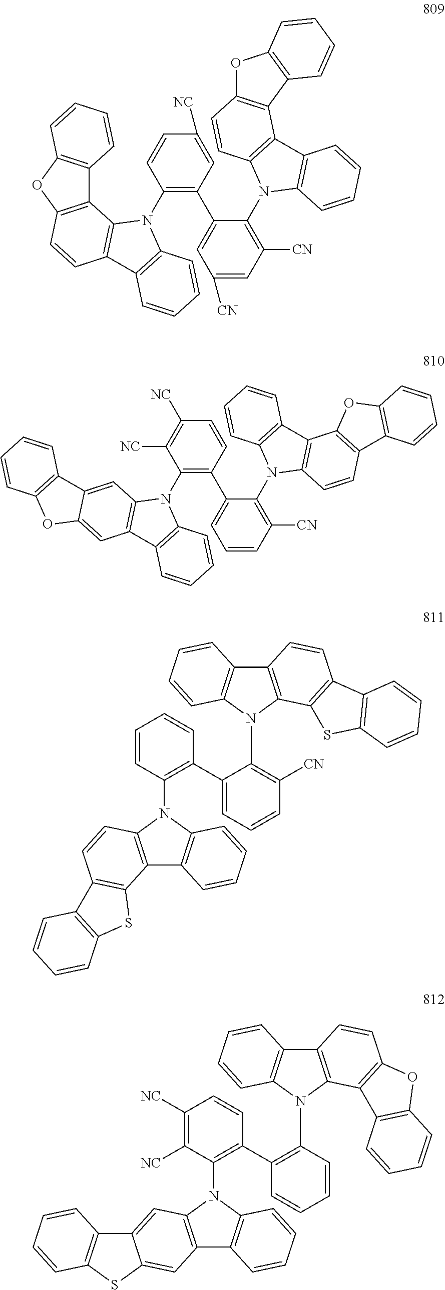 [SMILES: N#Cc1ccc(-n2c3ccccc3c3ccc4oc5ccccc5c4c32)c(-c2cc(C#N)cc(C#N)c2-n2c3ccccc3c3c4c(ccc32)oc2ccccc24)c1 ; N#Cc1ccc(-c2cccc(C#N)c2-n2c3ccccc3c3c4oc5ccccc5c4ccc32)c(-n2c3ccccc3c3cc4oc5ccccc5c4cc32)c1C#N ; N#Cc1cccc(-c2ccccc2-n2c3ccccc3c3c4sc5ccccc5c4ccc32)c1-n1c2ccccc2c2ccc3c4ccccc4sc3c21 ; N#Cc1ccc(-c2ccccc2-n2c3ccccc3c3ccc4oc5ccccc5c4c32)c(-n2c3ccccc3c3cc4sc5ccccc5c4cc32)c1C#N]

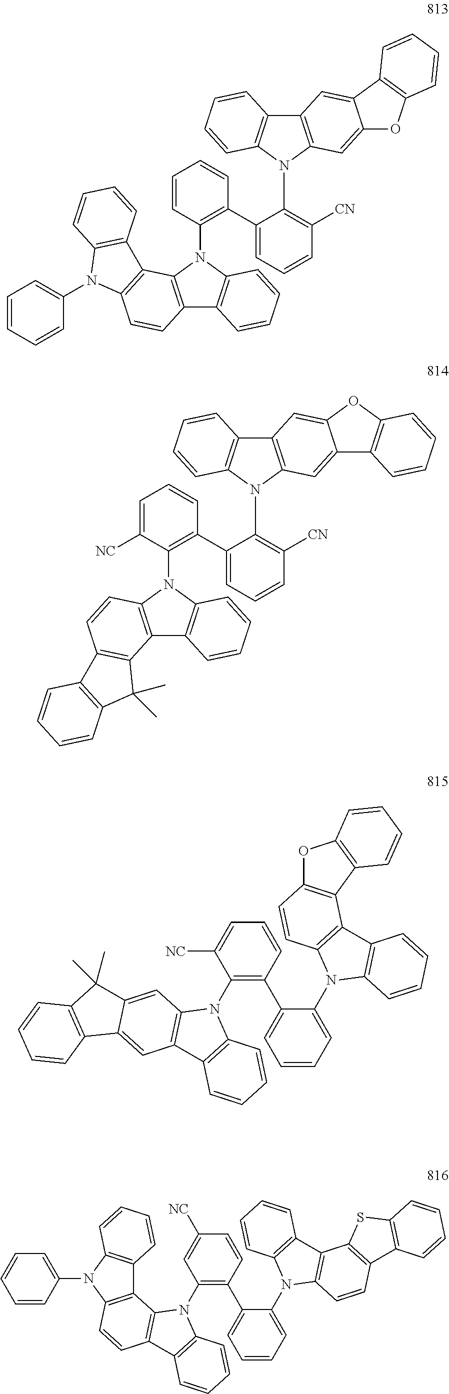 [SMILES: N#Cc1cccc(-c2ccccc2-n2c3ccccc3c3ccc4c(c5ccccc5n4-c4ccccc4)c32)c1-n1c2ccccc2c2cc3c(cc21)oc1ccccc13 ; CC1(C)c2ccccc2-c2ccc3c(c21)c1ccccc1n3-c1c(C#N)cccc1-c1cccc(C#N)c1-n1c2ccccc2c2cc3oc4ccccc4c3cc21 ; CC1(C)c2ccccc2-c2cc3c4ccccc4n(-c4c(C#N)cccc4-c4ccccc4-n4c5ccccc5c5c6c(ccc54)oc4ccccc46)c3cc21 ; N#Cc1ccc(-c2ccccc2-n2c3ccccc3c3c4sc5ccccc5c4ccc32)c(-n2c3ccccc3c3ccc4c(c5ccccc5n4-c4ccccc4)c32)c1]

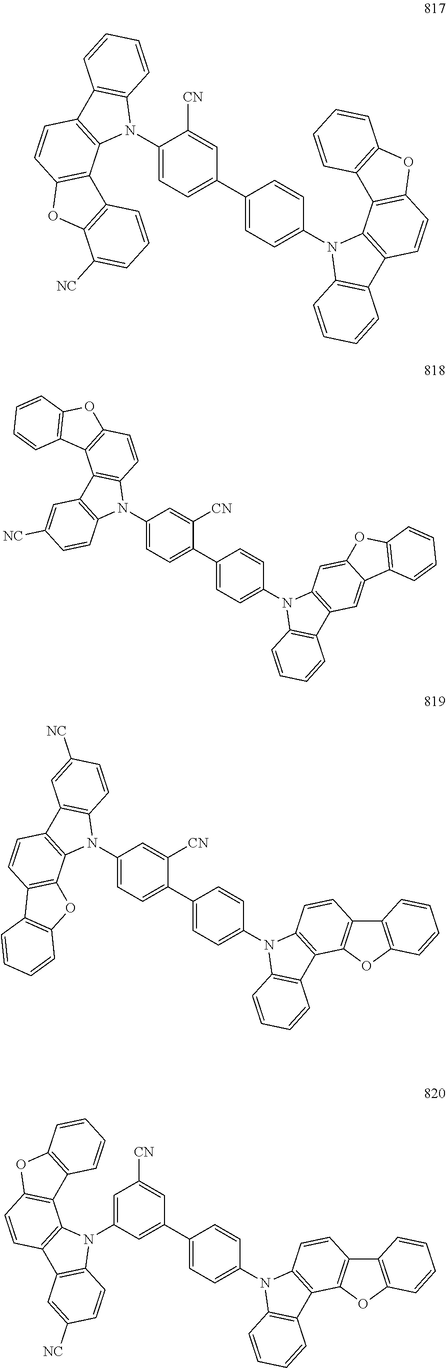 [SMILES: N#Cc1cc(-c2ccc(-n3c4ccccc4c4ccc5oc6ccccc6c5c43)cc2)ccc1-n1c2ccccc2c2ccc3oc4c(C#N)cccc4c3c21 ; N#Cc1ccc2c(c1)c1c3c(ccc1n2-c1ccc(-c2ccc(-n4c5ccccc5c5cc6c(cc54)oc4ccccc46)cc2)c(C#N)c1)oc1ccccc13 ; N#Cc1ccc2c(c1)c1ccc3c4ccccc4oc3c1n2-c1ccc(-c2ccc(-n3c4ccccc4c4c5oc6ccccc6c5ccc43)cc2)c(C#N)c1 ; N#Cc1cc(-c2ccc(-n3c4ccccc4c4c5oc6ccccc6c5ccc43)cc2)cc(-n2c3ccc(C#N)cc3c3ccc4oc5ccccc5c4c32)c1]

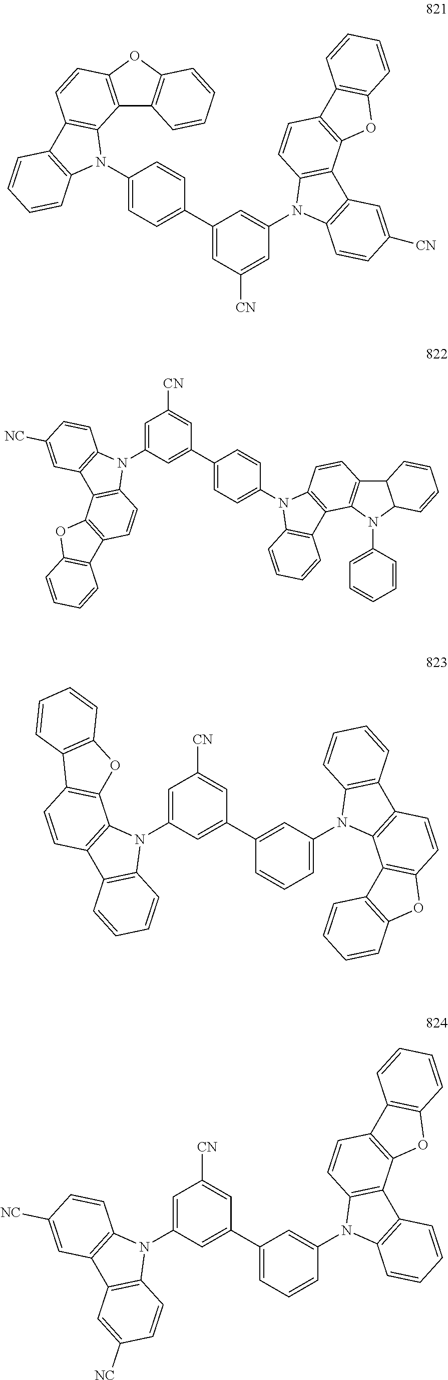 [SMILES: N#Cc1cc(-c2ccc(-n3c4ccccc4c4ccc5oc6ccccc6c5c43)cc2)cc(-n2c3ccc(C#N)cc3c3c4oc5ccccc5c4ccc32)c1 ; N#Cc1cc(-c2ccc(-n3c4ccccc4c4c5c(ccc43)C3C=CC=CC3N5c3ccccc3)cc2)cc(-n2c3ccc(C#N)cc3c3c4oc5ccccc5c4ccc32)c1 ; N#Cc1cc(-c2cccc(-n3c4ccccc4c4ccc5oc6ccccc6c5c43)c2)cc(-n2c3ccccc3c3ccc4c5ccccc5oc4c32)c1 ; N#Cc1cc(-c2cccc(-n3c4ccccc4c4c5oc6ccccc6c5ccc43)c2)cc(-n2c3ccc(C#N)cc3c3cc(C#N)ccc32)c1]

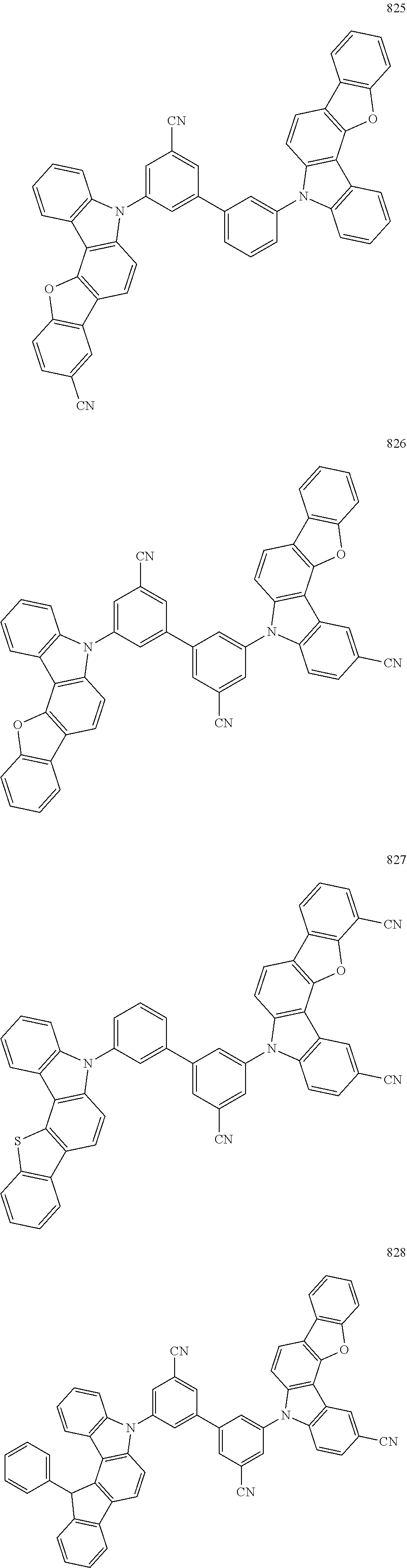 [SMILES: N#Cc1cc(-c2cccc(-n3c4ccccc4c4c5oc6ccccc6c5ccc43)c2)cc(-n2c3ccccc3c3c4oc5ccc(C#N)cc5c4ccc32)c1 ; N#Cc1cc(-c2cc(C#N)cc(-n3c4ccc(C#N)cc4c4c5oc6ccccc6c5ccc43)c2)cc(-n2c3ccccc3c3c4oc5ccccc5c4ccc32)c1 ; N#Cc1cc(-c2cccc(-n3c4ccccc4c4c5sc6ccccc6c5ccc43)c2)cc(-n2c3ccc(C#N)cc3c3c4oc5c(C#N)cccc5c4ccc32)c1 ; N#Cc1cc(-c2cc(C#N)cc(-n3c4ccc(C#N)cc4c4c5oc6ccccc6c5ccc43)c2)cc(-n2c3ccccc3c3c4c(ccc32)-c2ccccc2C4c2ccccc2)c1]

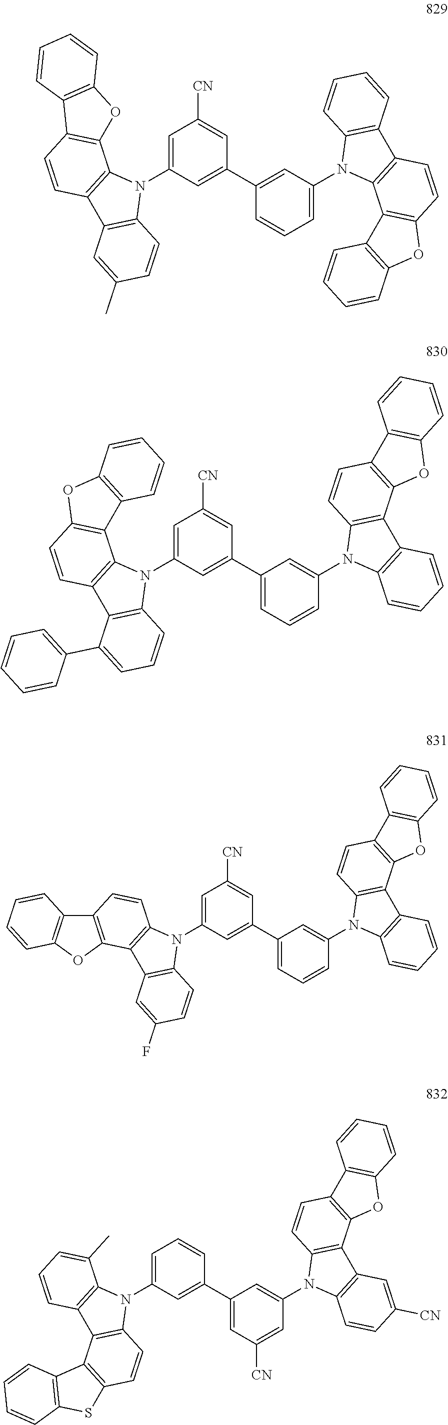 [SMILES: Cc1ccc2c(c1)c1ccc3c4ccccc4oc3c1n2-c1cc(C#N)cc(-c2cccc(-n3c4ccccc4c4ccc5oc6ccccc6c5c43)c2)c1 ; N#Cc1cc(-c2cccc(-n3c4ccccc4c4c5oc6ccccc6c5ccc43)c2)cc(-n2c3cccc(-c4ccccc4)c3c3ccc4oc5ccccc5c4c32)c1 ; N#Cc1cc(-c2cccc(-n3c4ccccc4c4c5oc6ccccc6c5ccc43)c2)cc(-n2c3ccc(F)cc3c3c4oc5ccccc5c4ccc32)c1 ; Cc1cccc2c3c4c(ccc3n(-c3cccc(-c5cc(C#N)cc(-n6c7ccc(C#N)cc7c7c8oc9ccccc9c8ccc76)c5)c3)c12)sc1ccccc14]

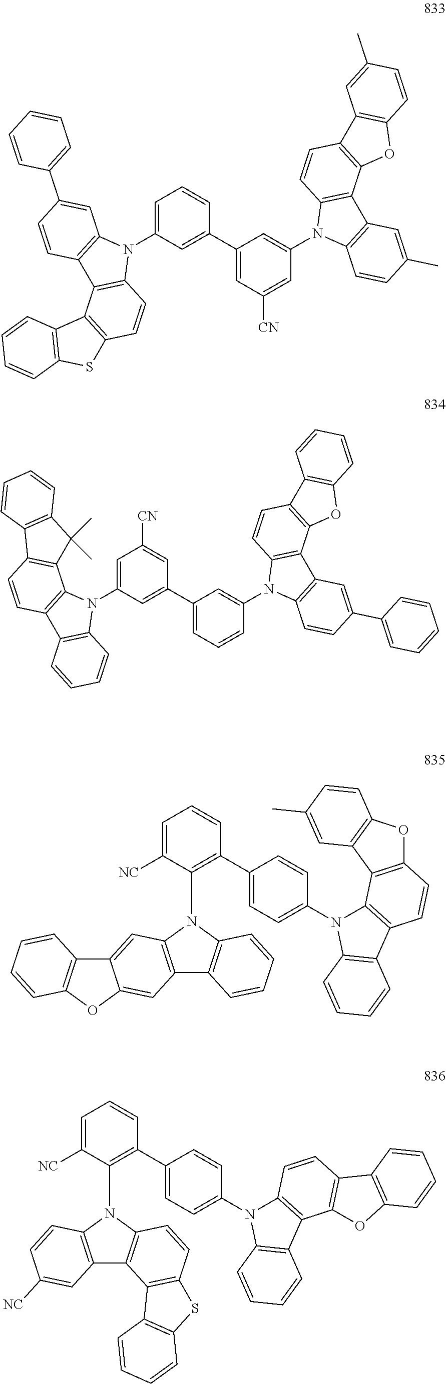 [SMILES: Cc1ccc2oc3c(ccc4c3c3cc(C)ccc3n4-c3cc(C#N)cc(-c4cccc(-n5c6cc(-c7ccccc7)ccc6c6c7c(ccc65)sc5ccccc57)c4)c3)c2c1 ; CC1(C)c2ccccc2-c2ccc3c4ccccc4n(-c4cc(C#N)cc(-c5cccc(-n6c7ccc(-c8ccccc8)cc7c7c8oc9ccccc9c8ccc76)c5)c4)c3c21 ; Cc1ccc2oc3ccc4c5ccccc5n(-c5ccc(-c6cccc(C#N)c6-n6c7ccccc7c7cc8oc9ccccc9c8cc76)cc5)c4c3c2c1 ; N#Cc1ccc2c(c1)c1c3c(ccc1n2-c1c(C#N)cccc1-c1ccc(-n2c4ccccc4c4c5oc6ccccc6c5ccc42)cc1)sc1ccccc13]

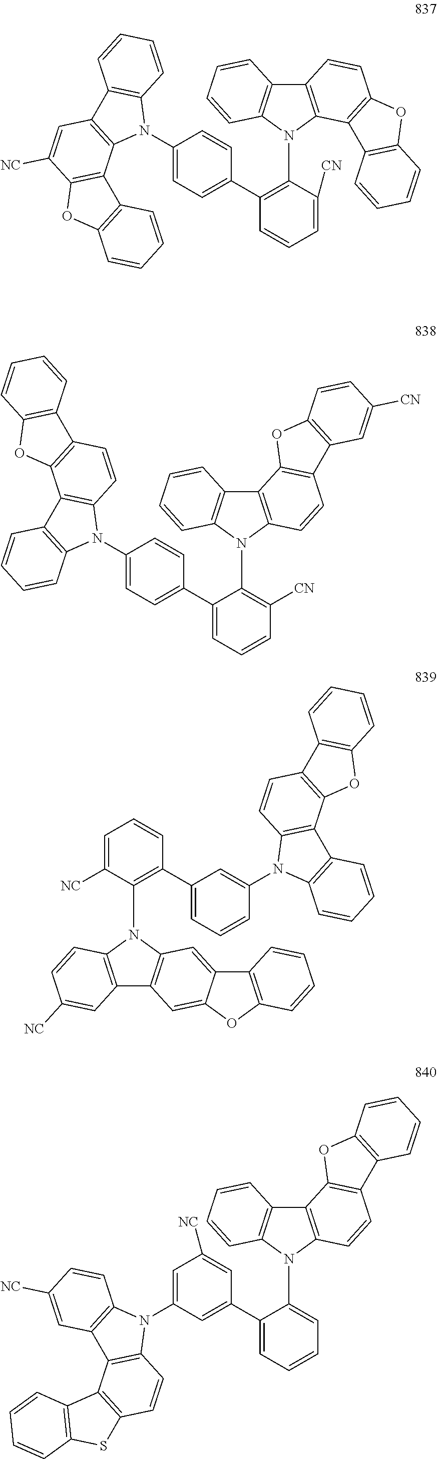 [SMILES: N#Cc1cccc(-c2ccc(-n3c4ccccc4c4cc(C#N)c5oc6ccccc6c5c43)cc2)c1-n1c2ccccc2c2ccc3oc4ccccc4c3c21 ; N#Cc1ccc2oc3c(ccc4c3c3ccccc3n4-c3c(C#N)cccc3-c3ccc(-n4c5ccccc5c5c6oc7ccccc7c6ccc54)cc3)c2c1 ; N#Cc1ccc2c(c1)c1cc3oc4ccccc4c3cc1n2-c1c(C#N)cccc1-c1cccc(-n2c3ccccc3c3c4oc5ccccc5c4ccc32)c1 ; N#Cc1cc(-c2ccccc2-n2c3ccccc3c3c4oc5ccccc5c4ccc32)cc(-n2c3ccc(C#N)cc3c3c4c(ccc32)sc2ccccc24)c1]

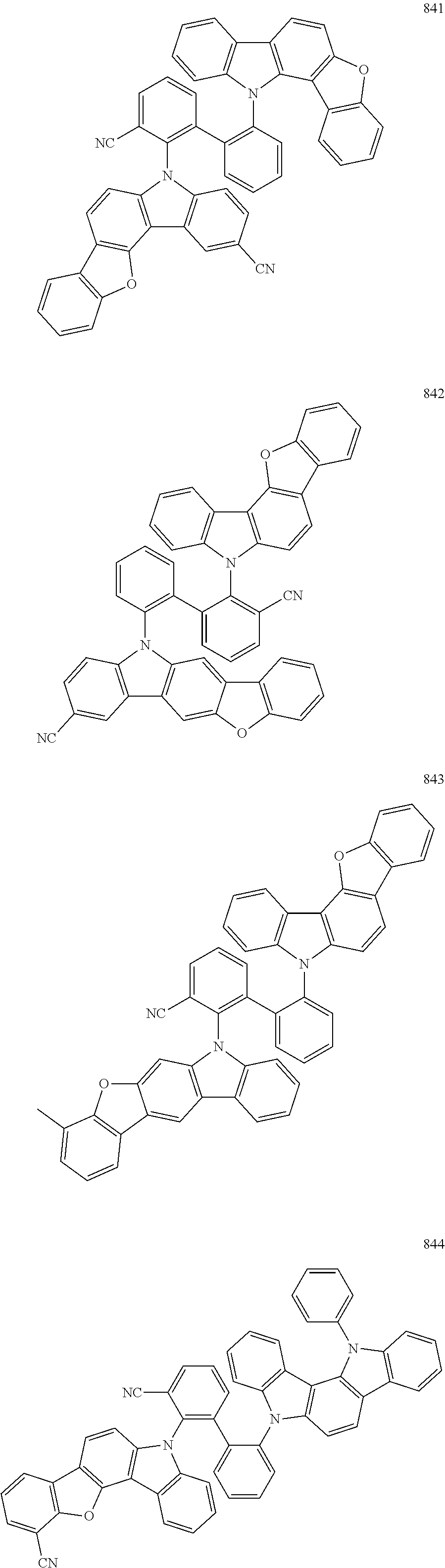 [SMILES: N#Cc1ccc2c(c1)c1c3oc4ccccc4c3ccc1n2-c1c(C#N)cccc1-c1ccccc1-n1c2ccccc2c2ccc3oc4ccccc4c3c21 ; N#Cc1ccc2c(c1)c1cc3oc4ccccc4c3cc1n2-c1ccccc1-c1cccc(C#N)c1-n1c2ccccc2c2c3oc4ccccc4c3ccc21 ; Cc1cccc2c1oc1cc3c(cc12)c1ccccc1n3-c1c(C#N)cccc1-c1ccccc1-n1c2ccccc2c2c3oc4ccccc4c3ccc21 ; N#Cc1cccc(-c2ccccc2-n2c3ccccc3c3c2ccc2c4ccccc4n(-c4ccccc4)c23)c1-n1c2ccccc2c2c3oc4c(C#N)cccc4c3ccc21]

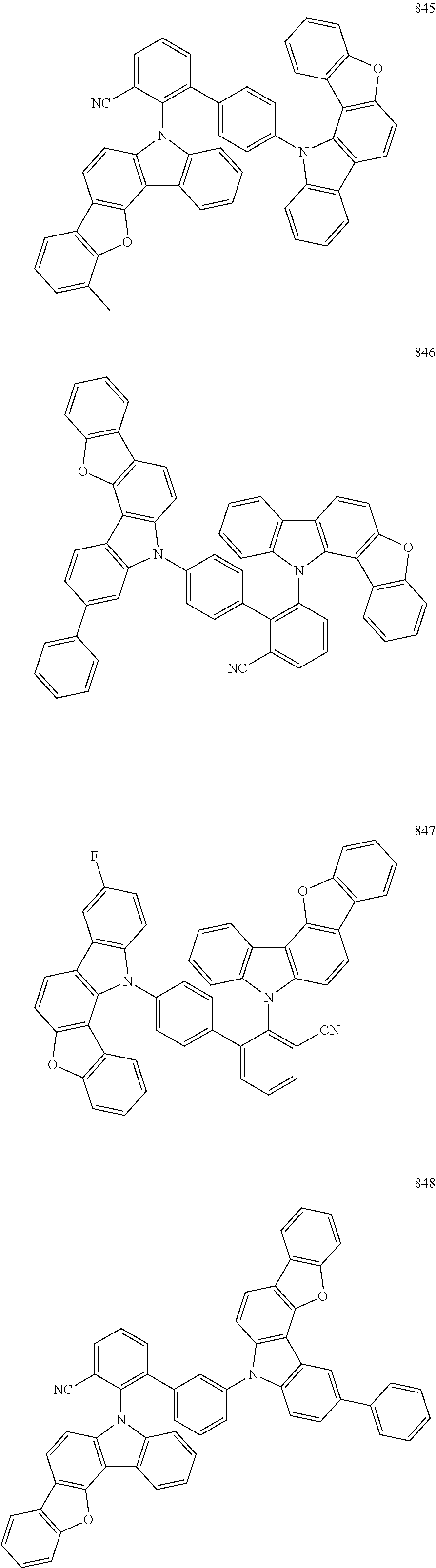 [SMILES: Cc1cccc2c1oc1c2ccc2c1c1ccccc1n2-c1c(C#N)cccc1-c1ccc(-n2c3ccccc3c3ccc4oc5ccccc5c4c32)cc1 ; N#Cc1cccc(-n2c3ccccc3c3ccc4oc5ccccc5c4c32)c1-c1ccc(-n2c3cc(-c4ccccc4)ccc3c3c4oc5ccccc5c4ccc32)cc1 ; N#Cc1cccc(-c2ccc(-n3c4ccc(F)cc4c4ccc5oc6ccccc6c5c43)cc2)c1-n1c2ccccc2c2c3oc4ccccc4c3ccc21 ; N#Cc1cccc(-c2cccc(-n3c4ccc(-c5ccccc5)cc4c4c5oc6ccccc6c5ccc43)c2)c1-n1c2ccccc2c2c3oc4ccccc4c3ccc21]

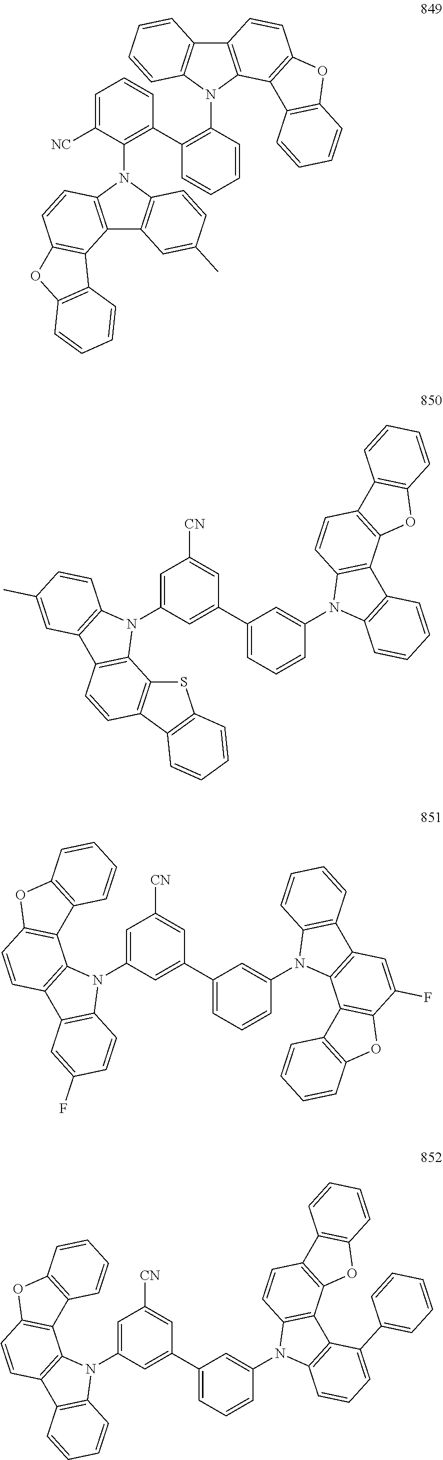 [SMILES: Cc1ccc2c(c1)c1c3c(ccc1n2-c1c(C#N)cccc1-c1ccccc1-n1c2ccccc2c2ccc4oc5ccccc5c4c21)oc1ccccc13 ; Cc1ccc2c(c1)c1ccc3c4ccccc4sc3c1n2-c1cc(C#N)cc(-c2cccc(-n3c4ccccc4c4c5oc6ccccc6c5ccc43)c2)c1 ; N#Cc1cc(-c2cccc(-n3c4ccccc4c4cc(F)c5oc6ccccc6c5c43)c2)cc(-n2c3ccc(F)cc3c3ccc4oc5ccccc5c4c32)c1 ; N#Cc1cc(-c2cccc(-n3c4cccc(-c5ccccc5)c4c4c5oc6ccccc6c5ccc43)c2)cc(-n2c3ccccc3c3ccc4oc5ccccc5c4c32)c1]

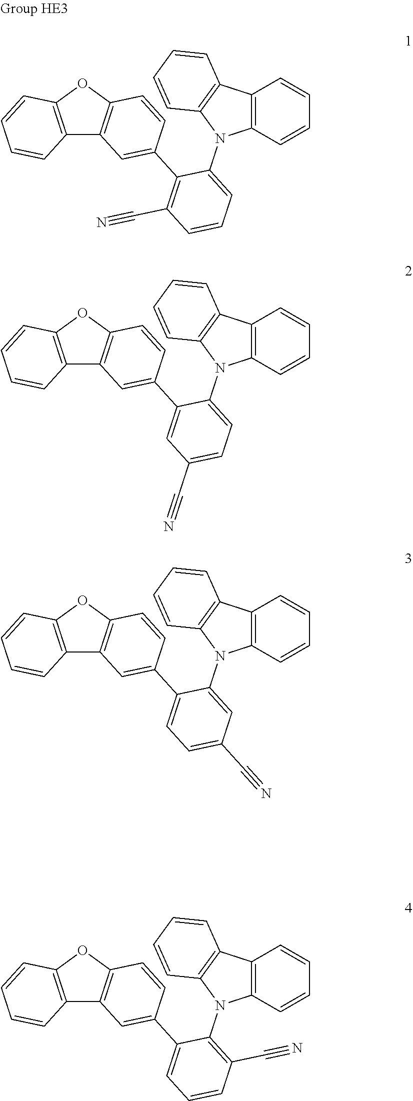

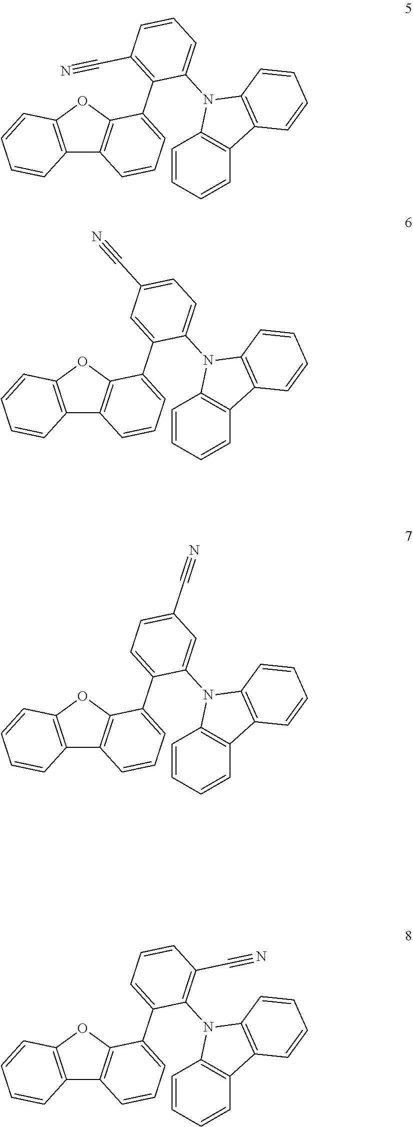 [SMILES: N#Cc1cccc(-n2c3ccccc3c3ccccc32)c1-c1cccc2c1oc1ccccc12 ; N#Cc1ccc(-n2c3ccccc3c3ccccc32)c(-c2cccc3c2oc2ccccc23)c1 ; N#Cc1ccc(-c2cccc3c2oc2ccccc23)c(-n2c3ccccc3c3ccccc32)c1 ; N#Cc1cccc(-c2cccc3c2oc2ccccc23)c1-n1c2ccccc2c2ccccc21]

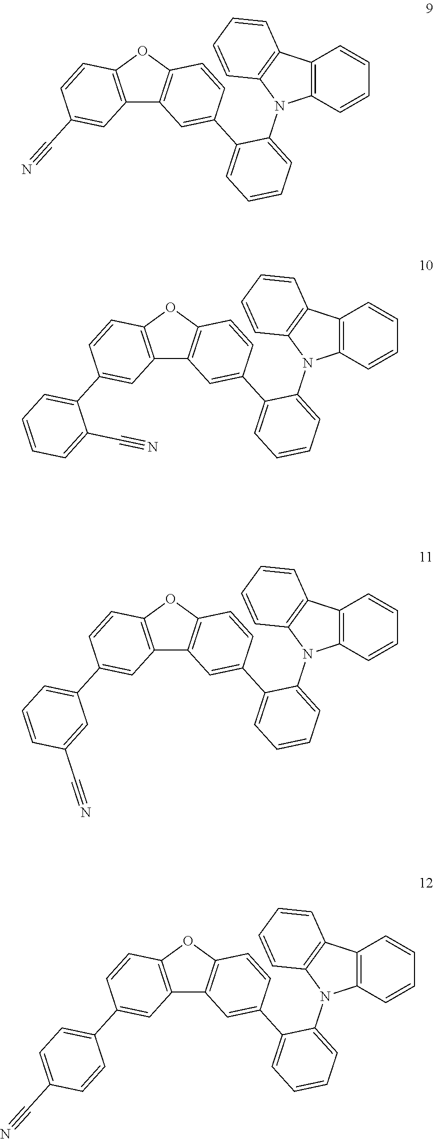 [SMILES: N#Cc1ccc2oc3ccc(-c4ccccc4-n4c5ccccc5c5ccccc54)cc3c2c1 ; N#Cc1ccccc1-c1ccc2oc3ccc(-c4ccccc4-n4c5ccccc5c5ccccc54)cc3c2c1 ; N#Cc1cccc(-c2ccc3oc4ccc(-c5ccccc5-n5c6ccccc6c6ccccc65)cc4c3c2)c1 ; N#Cc1ccc(-c2ccc3oc4ccc(-c5ccccc5-n5c6ccccc6c6ccccc65)cc4c3c2)cc1]

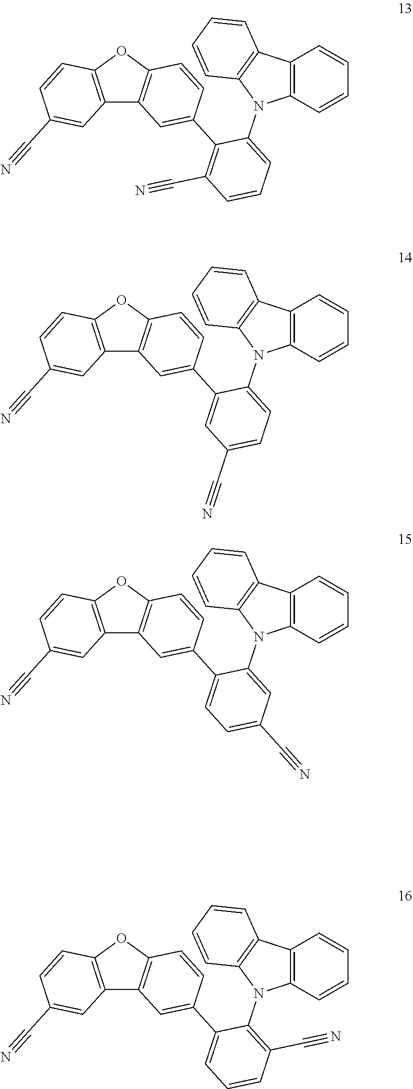

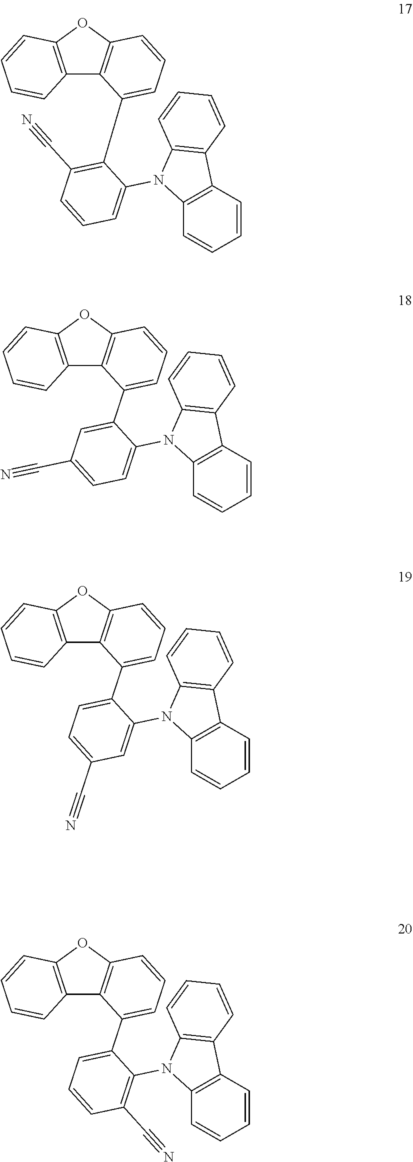 [SMILES: N#Cc1cccc(-n2c3ccccc3c3ccccc32)c1-c1cccc2oc3ccccc3c12 ; N#Cc1ccc(-n2c3ccccc3c3ccccc32)c(-c2cccc3oc4ccccc4c23)c1 ; N#Cc1ccc(-c2cccc3oc4ccccc4c23)c(-n2c3ccccc3c3ccccc32)c1 ; N#Cc1cccc(-c2cccc3oc4ccccc4c23)c1-n1c2ccccc2c2ccccc21]

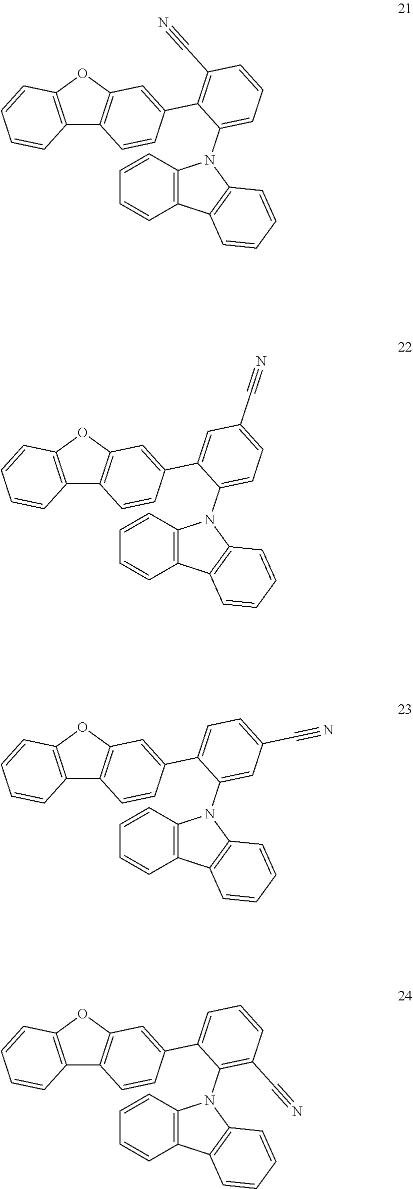 [SMILES: N#Cc1cccc(-n2c3ccccc3c3ccccc32)c1-c1ccc2c(c1)oc1ccccc12 ; N#Cc1ccc(-n2c3ccccc3c3ccccc32)c(-c2ccc3c(c2)oc2ccccc23)c1 ; N#Cc1ccc(-c2ccc3c(c2)oc2ccccc23)c(-n2c3ccccc3c3ccccc32)c1 ; N#Cc1cccc(-c2ccc3c(c2)oc2ccccc23)c1-n1c2ccccc2c2ccccc21]

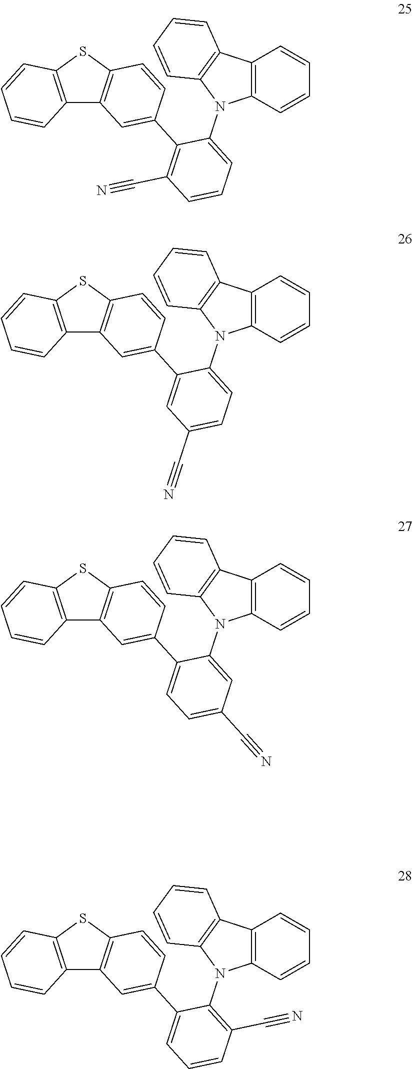

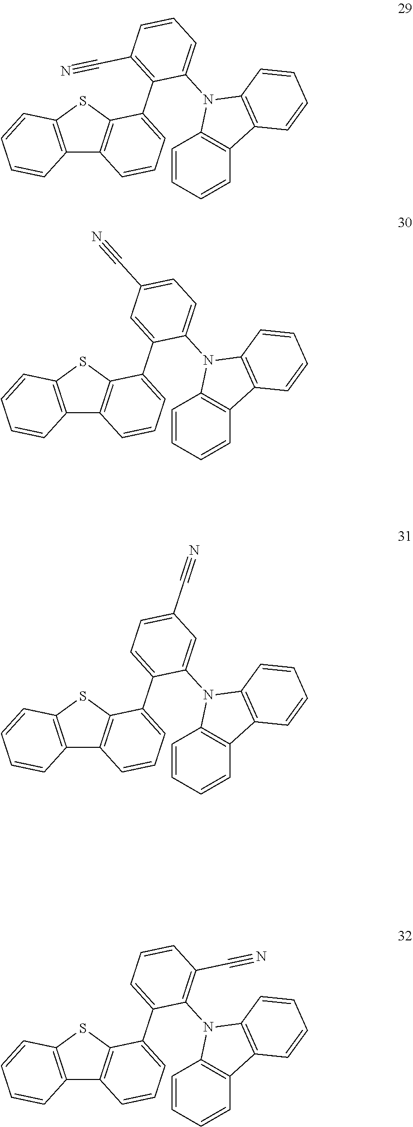 [SMILES: N#Cc1cccc(-n2c3ccccc3c3ccccc32)c1-c1cccc2c1sc1ccccc12 ; N#Cc1ccc(-n2c3ccccc3c3ccccc32)c(-c2cccc3c2sc2ccccc23)c1 ; N#Cc1ccc(-c2cccc3c2sc2ccccc23)c(-n2c3ccccc3c3ccccc32)c1 ; N#Cc1cccc(-c2cccc3c2sc2ccccc23)c1-n1c2ccccc2c2ccccc21]

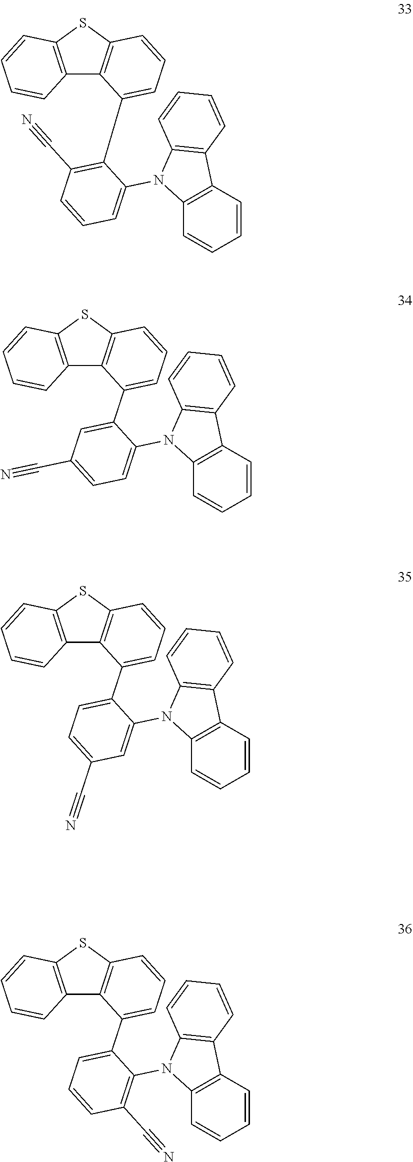 [SMILES: N#Cc1cccc(-n2c3ccccc3c3ccccc32)c1-c1cccc2sc3ccccc3c12 ; N#Cc1ccc(-n2c3ccccc3c3ccccc32)c(-c2cccc3sc4ccccc4c23)c1 ; N#Cc1ccc(-c2cccc3sc4ccccc4c23)c(-n2c3ccccc3c3ccccc32)c1 ; N#Cc1cccc(-c2cccc3sc4ccccc4c23)c1-n1c2ccccc2c2ccccc21]

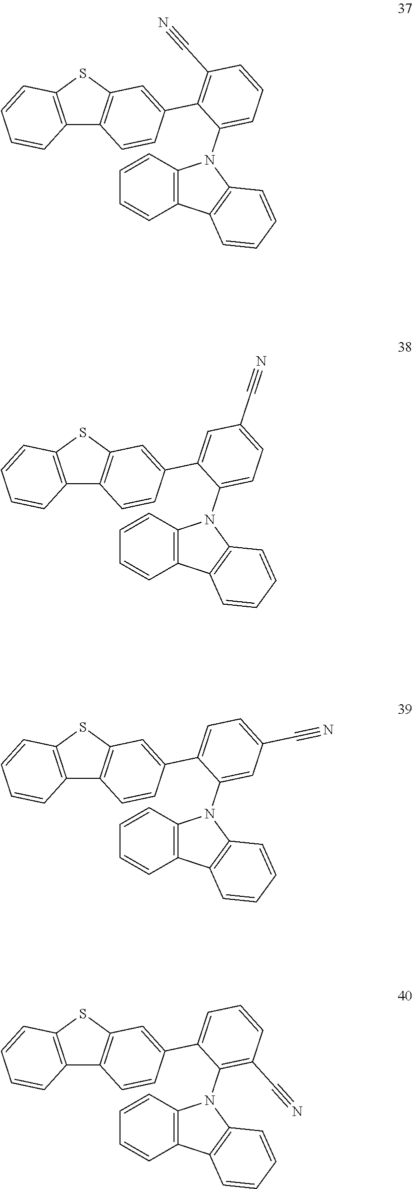 [SMILES: N#Cc1cccc(-n2c3ccccc3c3ccccc32)c1-c1ccc2c(c1)sc1ccccc12 ; N#Cc1ccc(-n2c3ccccc3c3ccccc32)c(-c2ccc3c(c2)sc2ccccc23)c1 ; N#Cc1ccc(-c2ccc3c(c2)sc2ccccc23)c(-n2c3ccccc3c3ccccc32)c1 ; N#Cc1cccc(-c2ccc3c(c2)sc2ccccc23)c1-n1c2ccccc2c2ccccc21]

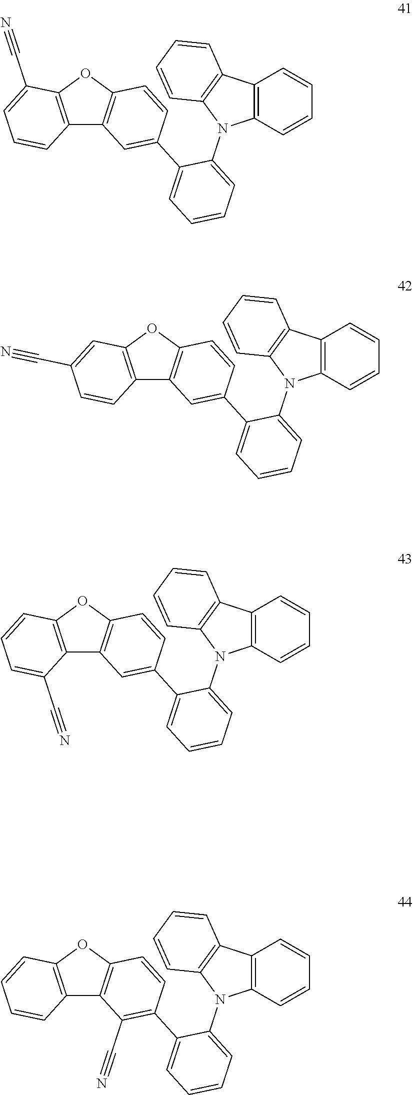 [SMILES: N#Cc1cccc2c1oc1ccc(-c3ccccc3-n3c4ccccc4c4ccccc43)cc12 ; N#Cc1ccc2c(c1)oc1ccc(-c3ccccc3-n3c4ccccc4c4ccccc43)cc12 ; N#Cc1cccc2oc3ccc(-c4ccccc4-n4c5ccccc5c5ccccc54)cc3c12 ; N#Cc1c(-c2ccccc2-n2c3ccccc3c3ccccc32)ccc2oc3ccccc3c12]

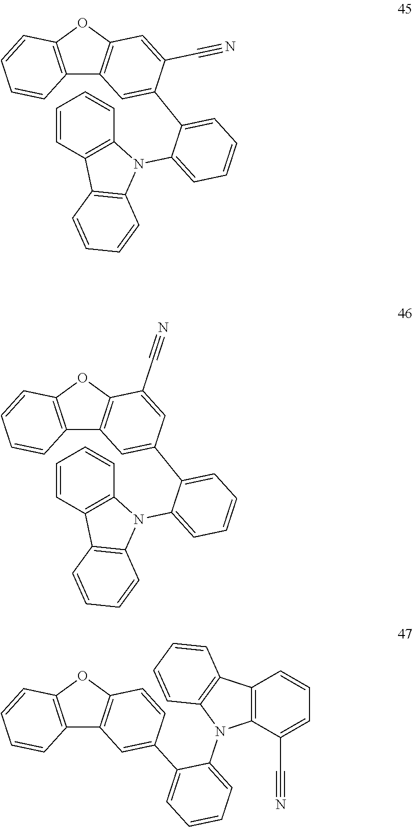 [SMILES: N#Cc1cc2oc3ccccc3c2cc1-c1ccccc1-n1c2ccccc2c2ccccc21 ; N#Cc1cc(-c2ccccc2-n2c3ccccc3c3ccccc32)cc2c1oc1ccccc12 ; N#Cc1cccc2c3ccccc3n(-c3ccccc3-c3ccc4oc5ccccc5c4c3)c12]

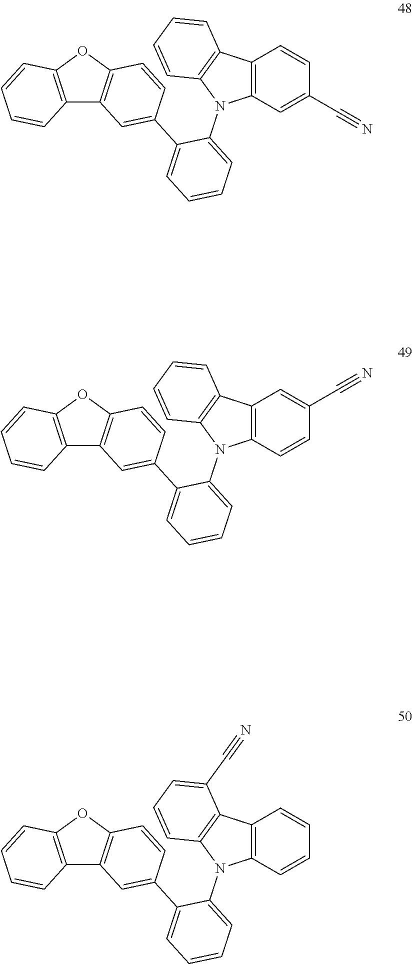 [SMILES: N#Cc1ccc2c3ccccc3n(-c3ccccc3-c3ccc4oc5ccccc5c4c3)c2c1 ; N#Cc1ccc2c(c1)c1ccccc1n2-c1ccccc1-c1ccc2oc3ccccc3c2c1 ; N#Cc1cccc2c1c1ccccc1n2-c1ccccc1-c1ccc2oc3ccccc3c2c1]

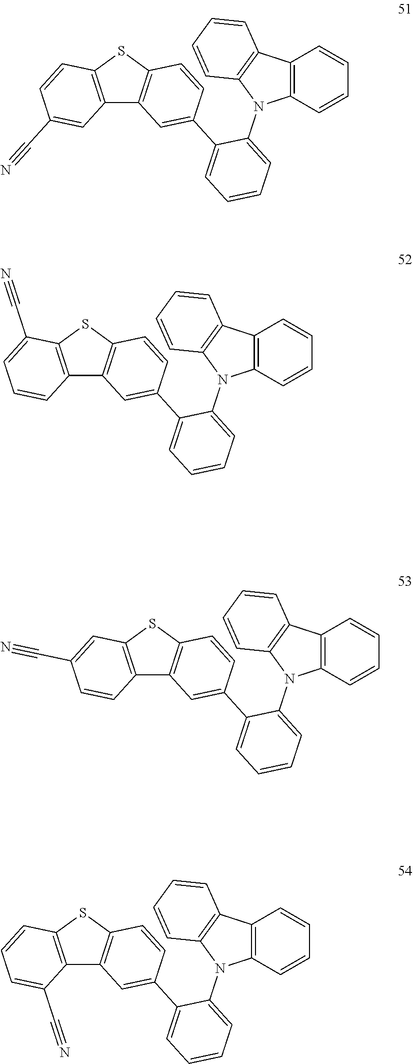 [SMILES: N#Cc1ccc2sc3ccc(-c4ccccc4-n4c5ccccc5c5ccccc54)cc3c2c1 ; N#Cc1cccc2c1sc1ccc(-c3ccccc3-n3c4ccccc4c4ccccc43)cc12 ; N#Cc1ccc2c(c1)sc1ccc(-c3ccccc3-n3c4ccccc4c4ccccc43)cc12 ; N#Cc1cccc2sc3ccc(-c4ccccc4-n4c5ccccc5c5ccccc54)cc3c12]

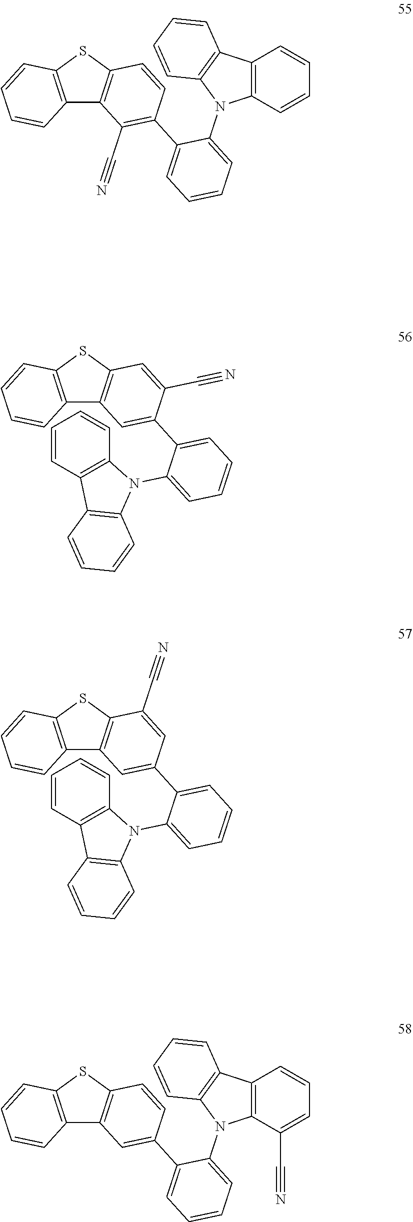 [SMILES: N#Cc1c(-c2ccccc2-n2c3ccccc3c3ccccc32)ccc2sc3ccccc3c12 ; N#Cc1cc2sc3ccccc3c2cc1-c1ccccc1-n1c2ccccc2c2ccccc21 ; N#Cc1cc(-c2ccccc2-n2c3ccccc3c3ccccc32)cc2c1sc1ccccc12 ; N#Cc1cccc2c3ccccc3n(-c3ccccc3-c3ccc4sc5ccccc5c4c3)c12]

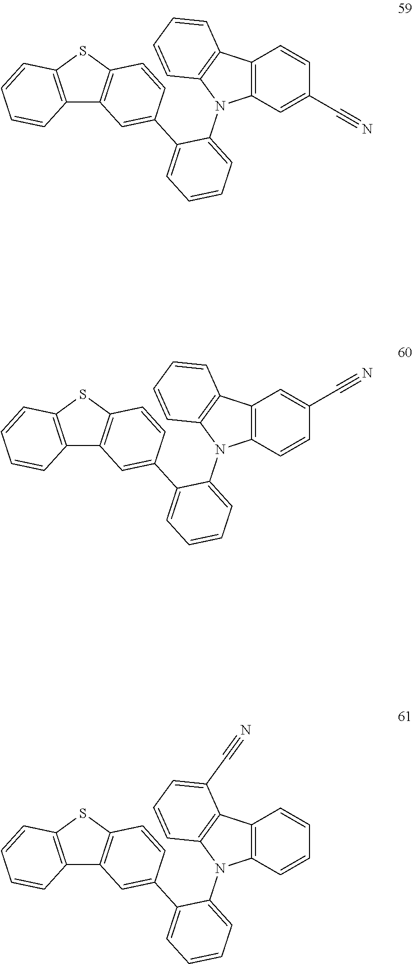 [SMILES: N#Cc1ccc2c3ccccc3n(-c3ccccc3-c3ccc4sc5ccccc5c4c3)c2c1 ; N#Cc1ccc2c(c1)c1ccccc1n2-c1ccccc1-c1ccc2sc3ccccc3c2c1 ; N#Cc1cccc2c1c1ccccc1n2-c1ccccc1-c1ccc2sc3ccccc3c2c1]

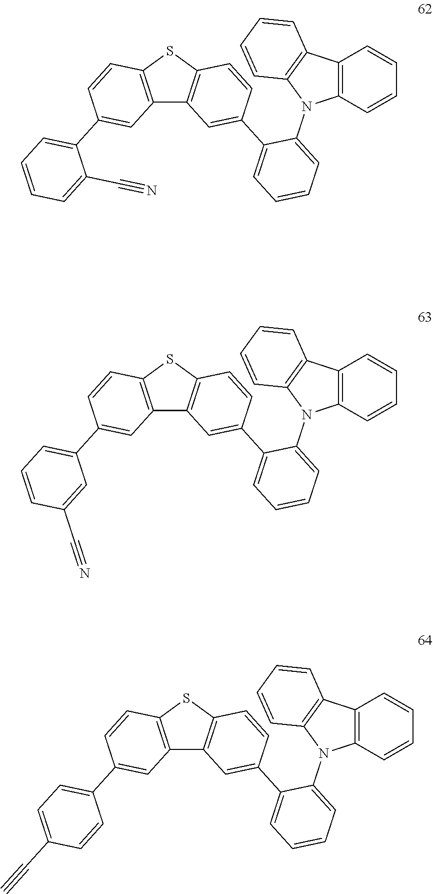 [SMILES: N#Cc1ccccc1-c1ccc2sc3ccc(-c4ccccc4-n4c5ccccc5c5ccccc54)cc3c2c1 ; N#Cc1cccc(-c2ccc3sc4ccc(-c5ccccc5-n5c6ccccc6c6ccccc65)cc4c3c2)c1 ; C#Cc1ccc(-c2ccc3sc4ccc(-c5ccccc5-n5c6ccccc6c6ccccc65)cc4c3c2)cc1]

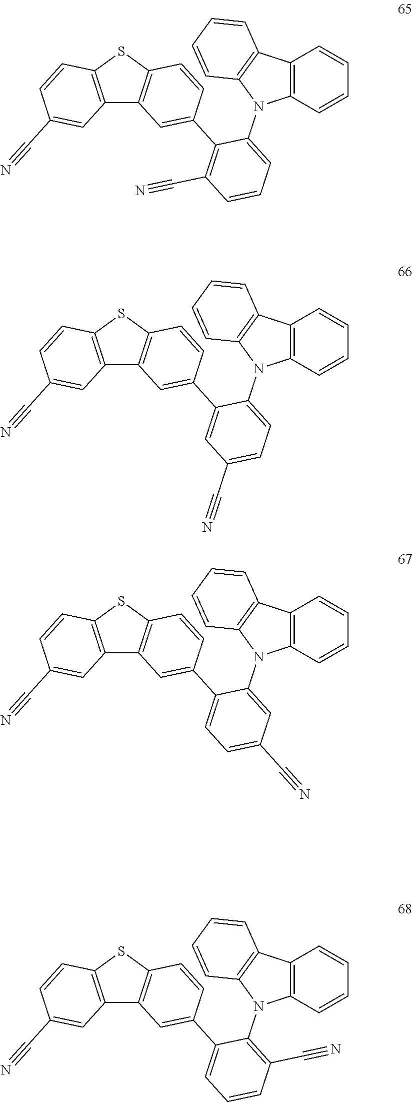

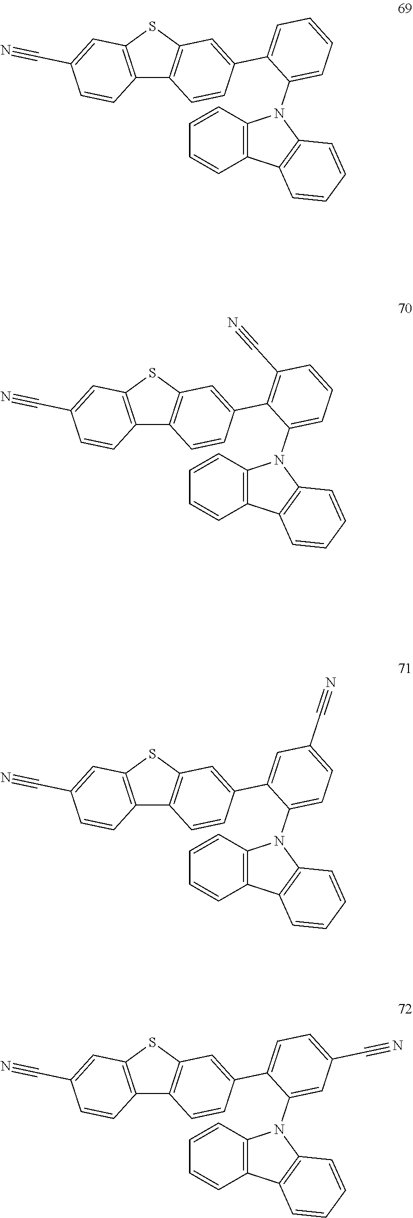 [SMILES: N#Cc1ccc2c(c1)sc1cc(-c3ccccc3-n3c4ccccc4c4ccccc43)ccc12 ; N#Cc1ccc2c(c1)sc1cc(-c3c(C#N)cccc3-n3c4ccccc4c4ccccc43)ccc12 ; N#Cc1ccc(-n2c3ccccc3c3ccccc32)c(-c2ccc3c(c2)sc2cc(C#N)ccc23)c1 ; N#Cc1ccc(-c2ccc3c(c2)sc2cc(C#N)ccc23)c(-n2c3ccccc3c3ccccc32)c1]

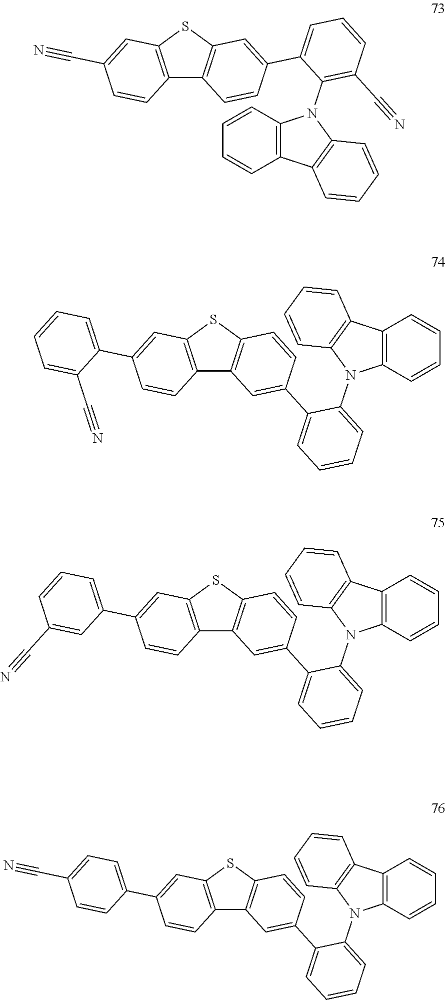 [SMILES: N#Cc1ccc2c(c1)sc1cc(-c3cccc(C#N)c3-n3c4ccccc4c4ccccc43)ccc12 ; N#Cc1ccccc1-c1ccc2c(c1)sc1ccc(-c3ccccc3-n3c4ccccc4c4ccccc43)cc12 ; N#Cc1cccc(-c2ccc3c(c2)sc2ccc(-c4ccccc4-n4c5ccccc5c5ccccc54)cc23)c1 ; N#Cc1ccc(-c2ccc3c(c2)sc2ccc(-c4ccccc4-n4c5ccccc5c5ccccc54)cc23)cc1]

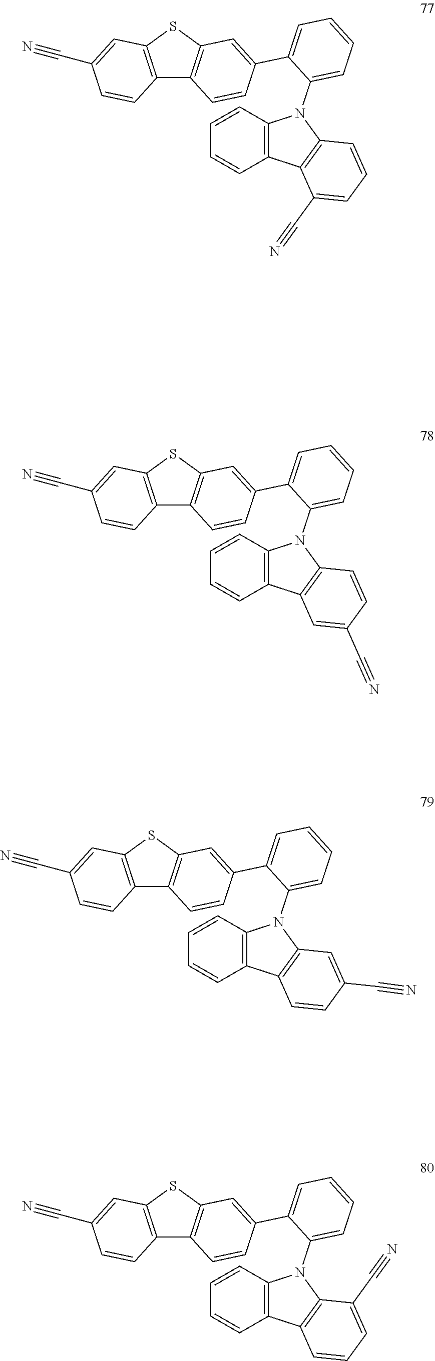 [SMILES: N#Cc1ccc2c(c1)sc1cc(-c3ccccc3-n3c4ccccc4c4c(C#N)cccc43)ccc12 ; N#Cc1ccc2c(c1)sc1cc(-c3ccccc3-n3c4ccccc4c4cc(C#N)ccc43)ccc12 ; N#Cc1ccc2c(c1)sc1cc(-c3ccccc3-n3c4ccccc4c4ccc(C#N)cc43)ccc12 ; N#Cc1ccc2c(c1)sc1cc(-c3ccccc3-n3c4ccccc4c4cccc(C#N)c43)ccc12]

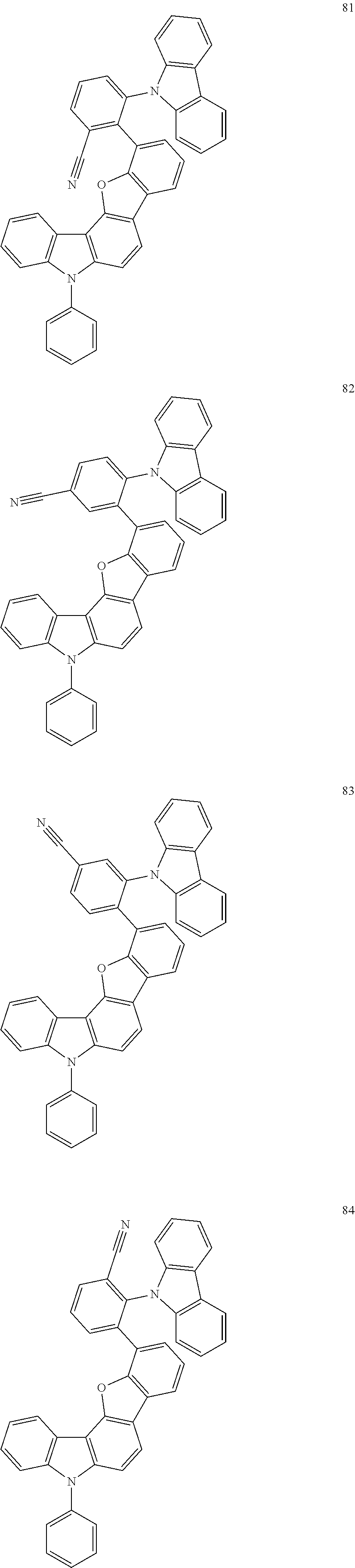 [SMILES: N#Cc1cccc(-n2c3ccccc3c3ccccc32)c1-c1cccc2c1oc1c2ccc2c1c1ccccc1n2-c1ccccc1 ; N#Cc1ccc(-n2c3ccccc3c3ccccc32)c(-c2cccc3c2oc2c3ccc3c2c2ccccc2n3-c2ccccc2)c1 ; N#Cc1ccc(-c2cccc3c2oc2c3ccc3c2c2ccccc2n3-c2ccccc2)c(-n2c3ccccc3c3ccccc32)c1 ; N#Cc1cccc(-c2cccc3c2oc2c3ccc3c2c2ccccc2n3-c2ccccc2)c1-n1c2ccccc2c2ccccc21]

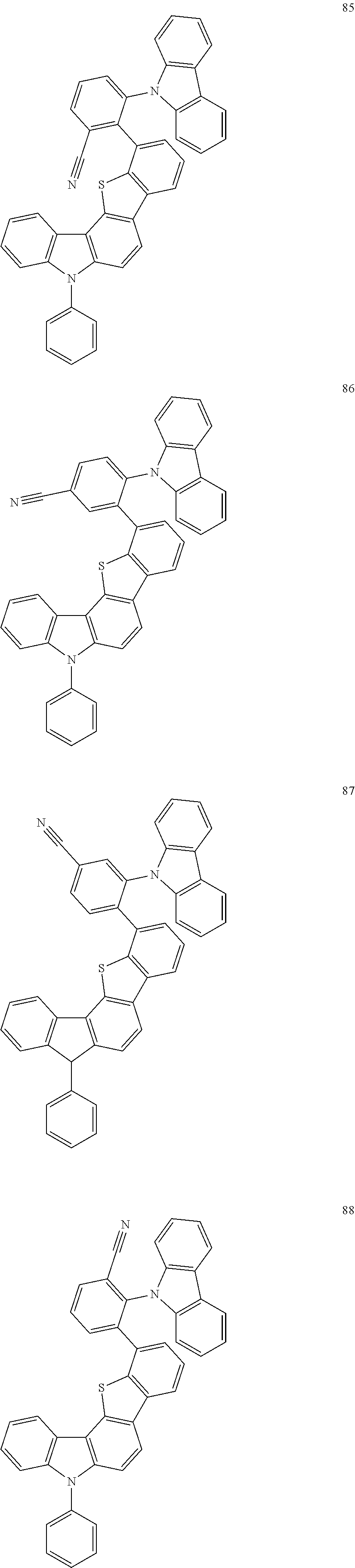 [SMILES: N#Cc1cccc(-n2c3ccccc3c3ccccc32)c1-c1cccc2c1sc1c2ccc2c1c1ccccc1n2-c1ccccc1 ; N#Cc1ccc(-n2c3ccccc3c3ccccc32)c(-c2cccc3c2sc2c3ccc3c2c2ccccc2n3-c2ccccc2)c1 ; N#Cc1ccc(-c2cccc3c2sc2c4c(ccc23)C(c2ccccc2)c2ccccc2-4)c(-n2c3ccccc3c3ccccc32)c1 ; N#Cc1cccc(-c2cccc3c2sc2c3ccc3c2c2ccccc2n3-c2ccccc2)c1-n1c2ccccc2c2ccccc21]

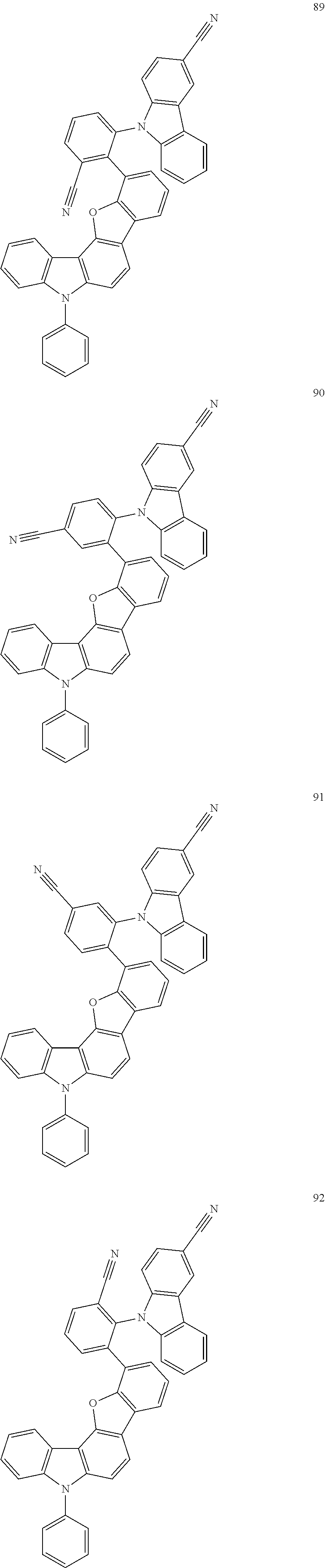 [SMILES: N#Cc1ccc2c(c1)c1ccccc1n2-c1cccc(C#N)c1-c1cccc2c1oc1c2ccc2c1c1ccccc1n2-c1ccccc1 ; N#Cc1ccc(-n2c3ccccc3c3cc(C#N)ccc32)c(-c2cccc3c2oc2c3ccc3c2c2ccccc2n3-c2ccccc2)c1 ; N#Cc1ccc(-c2cccc3c2oc2c3ccc3c2c2ccccc2n3-c2ccccc2)c(-n2c3ccccc3c3cc(C#N)ccc32)c1 ; N#Cc1ccc2c(c1)c1ccccc1n2-c1c(C#N)cccc1-c1cccc2c1oc1c2ccc2c1c1ccccc1n2-c1ccccc1]

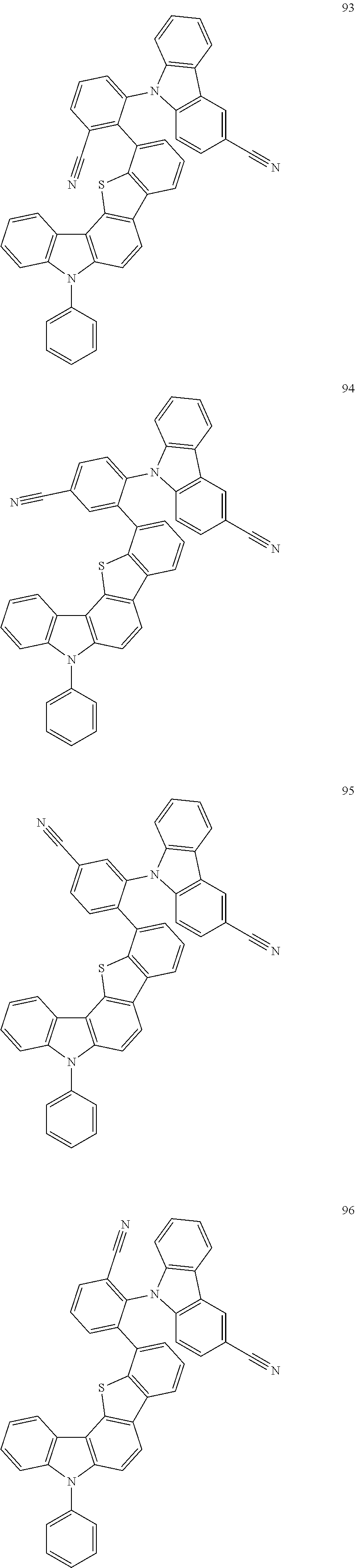 [SMILES: N#Cc1ccc2c(c1)c1ccccc1n2-c1cccc(C#N)c1-c1cccc2c1sc1c2ccc2c1c1ccccc1n2-c1ccccc1 ; N#Cc1ccc(-n2c3ccccc3c3cc(C#N)ccc32)c(-c2cccc3c2sc2c3ccc3c2c2ccccc2n3-c2ccccc2)c1 ; N#Cc1ccc(-c2cccc3c2sc2c3ccc3c2c2ccccc2n3-c2ccccc2)c(-n2c3ccccc3c3cc(C#N)ccc32)c1 ; N#Cc1ccc2c(c1)c1ccccc1n2-c1c(C#N)cccc1-c1cccc2c1sc1c2ccc2c1c1ccccc1n2-c1ccccc1]

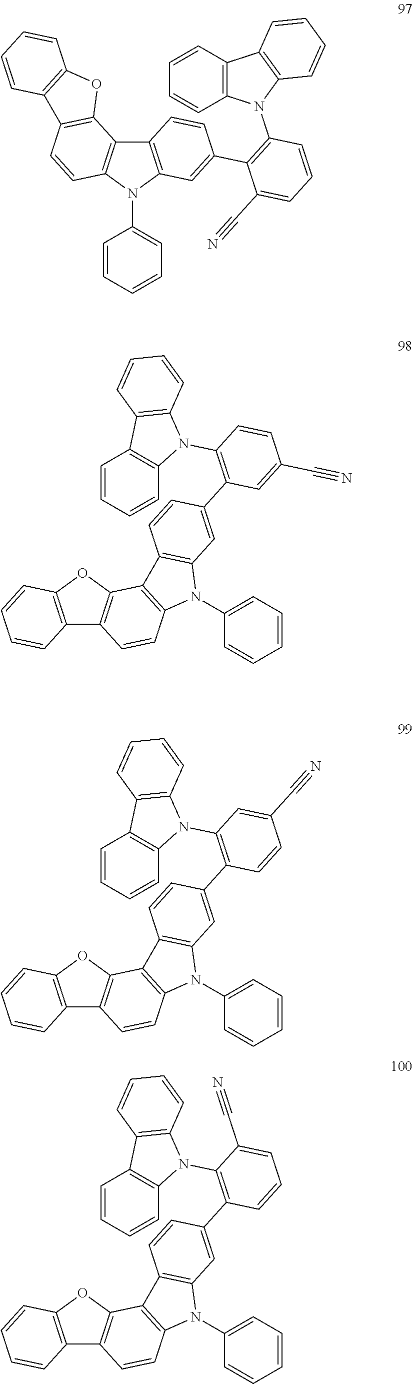 [SMILES: N#Cc1cccc(-n2c3ccccc3c3ccccc32)c1-c1ccc2c3c4oc5ccccc5c4ccc3n(-c3ccccc3)c2c1 ; N#Cc1ccc(-n2c3ccccc3c3ccccc32)c(-c2ccc3c4c5oc6ccccc6c5ccc4n(-c4ccccc4)c3c2)c1 ; N#Cc1ccc(-c2ccc3c4c5oc6ccccc6c5ccc4n(-c4ccccc4)c3c2)c(-n2c3ccccc3c3ccccc32)c1 ; N#Cc1cccc(-c2ccc3c4c5oc6ccccc6c5ccc4n(-c4ccccc4)c3c2)c1-n1c2ccccc2c2ccccc21]

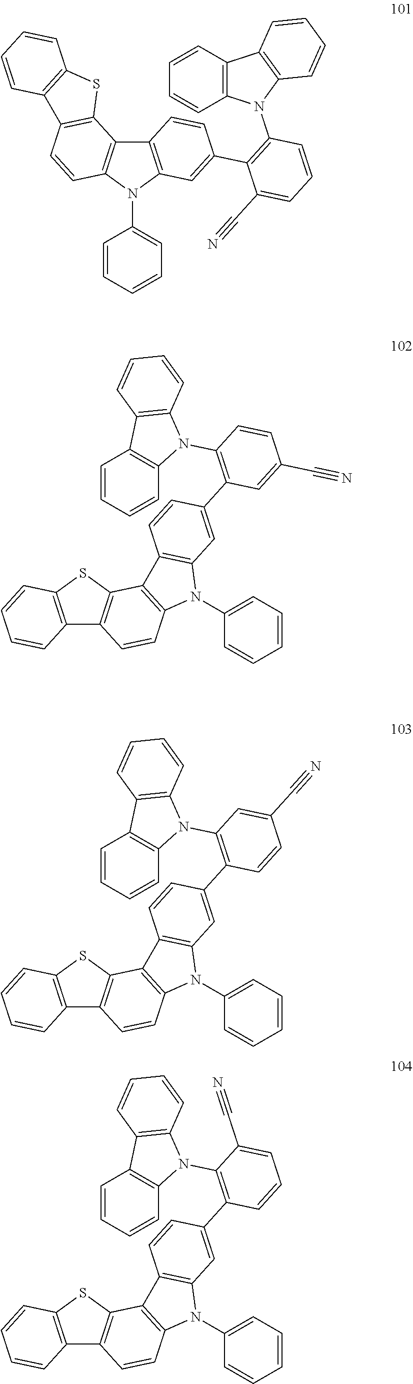 [SMILES: N#Cc1cccc(-n2c3ccccc3c3ccccc32)c1-c1ccc2c3c4sc5ccccc5c4ccc3n(-c3ccccc3)c2c1 ; N#Cc1ccc(-n2c3ccccc3c3ccccc32)c(-c2ccc3c4c5sc6ccccc6c5ccc4n(-c4ccccc4)c3c2)c1 ; N#Cc1ccc(-c2ccc3c4c5sc6ccccc6c5ccc4n(-c4ccccc4)c3c2)c(-n2c3ccccc3c3ccccc32)c1 ; N#Cc1cccc(-c2ccc3c4c5sc6ccccc6c5ccc4n(-c4ccccc4)c3c2)c1-n1c2ccccc2c2ccccc21]

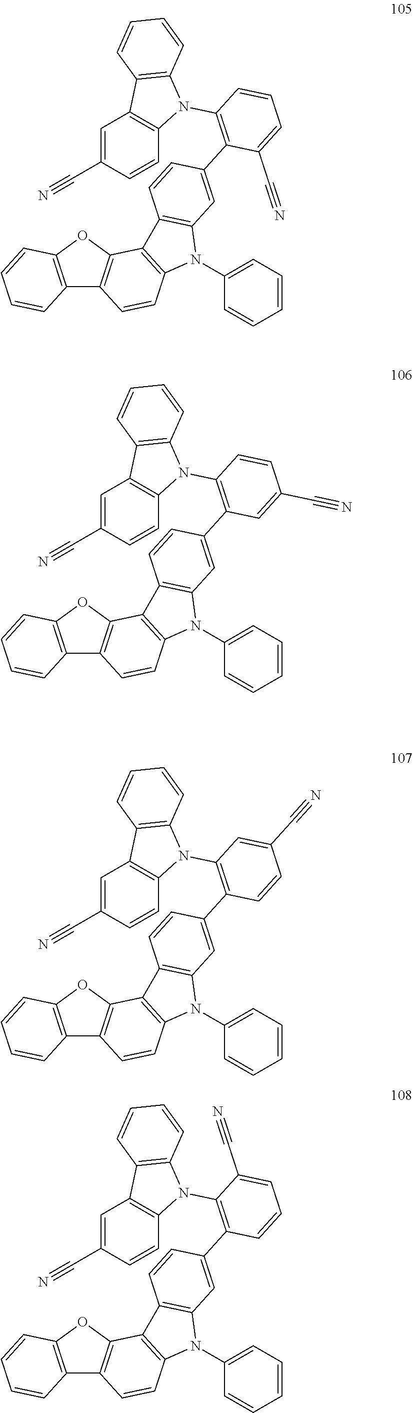 [SMILES: N#Cc1ccc2c(c1)c1ccccc1n2-c1cccc(C#N)c1-c1ccc2c3c4oc5ccccc5c4ccc3n(-c3ccccc3)c2c1 ; N#Cc1ccc(-n2c3ccccc3c3cc(C#N)ccc32)c(-c2ccc3c4c5oc6ccccc6c5ccc4n(-c4ccccc4)c3c2)c1 ; N#Cc1ccc(-c2ccc3c4c5oc6ccccc6c5ccc4n(-c4ccccc4)c3c2)c(-n2c3ccccc3c3cc(C#N)ccc32)c1 ; N#Cc1ccc2c(c1)c1ccccc1n2-c1c(C#N)cccc1-c1ccc2c3c4oc5ccccc5c4ccc3n(-c3ccccc3)c2c1]

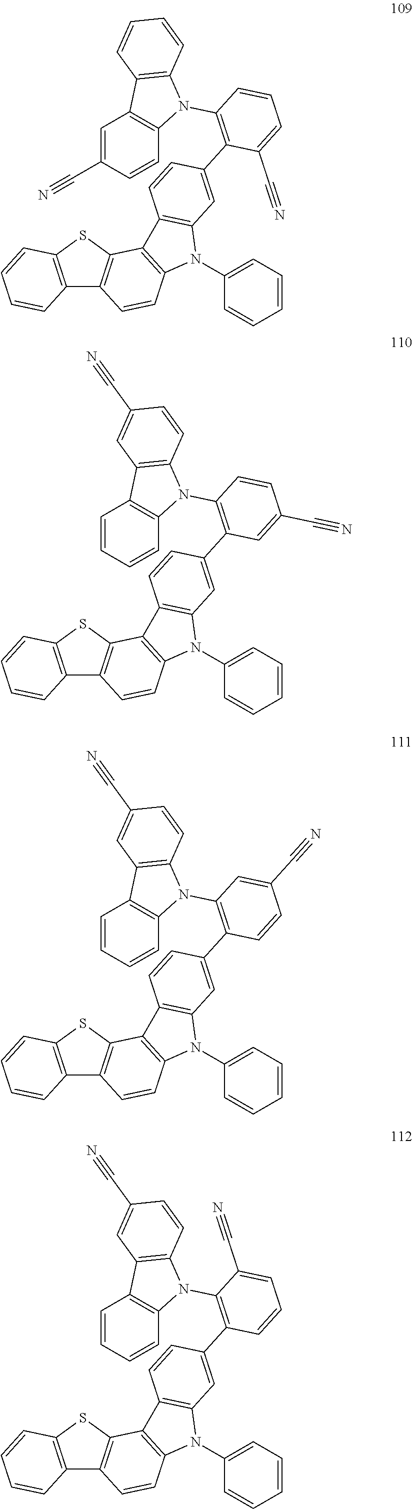 [SMILES: N#Cc1ccc2c(c1)c1ccccc1n2-c1cccc(C#N)c1-c1ccc2c3c4sc5ccccc5c4ccc3n(-c3ccccc3)c2c1 ; N#Cc1ccc(-n2c3ccccc3c3cc(C#N)ccc32)c(-c2ccc3c4c5sc6ccccc6c5ccc4n(-c4ccccc4)c3c2)c1 ; N#Cc1ccc(-c2ccc3c4c5sc6ccccc6c5ccc4n(-c4ccccc4)c3c2)c(-n2c3ccccc3c3cc(C#N)ccc32)c1 ; N#Cc1ccc2c(c1)c1ccccc1n2-c1c(C#N)cccc1-c1ccc2c3c4sc5ccccc5c4ccc3n(-c3ccccc3)c2c1]

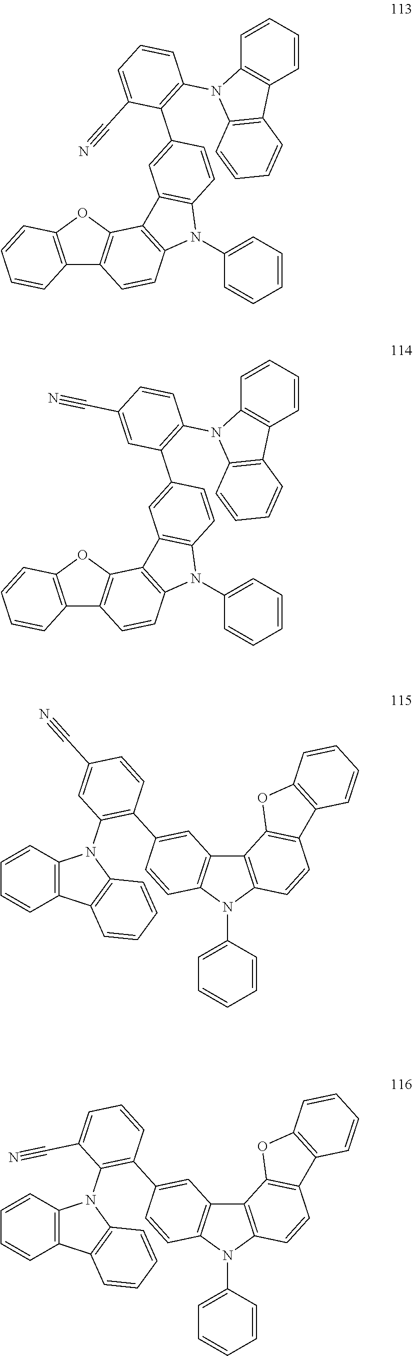 [SMILES: N#Cc1cccc(-n2c3ccccc3c3ccccc32)c1-c1ccc2c(c1)c1c3oc4ccccc4c3ccc1n2-c1ccccc1 ; N#Cc1ccc(-n2c3ccccc3c3ccccc32)c(-c2ccc3c(c2)c2c4oc5ccccc5c4ccc2n3-c2ccccc2)c1 ; N#Cc1ccc(-c2ccc3c(c2)c2c4oc5ccccc5c4ccc2n3-c2ccccc2)c(-n2c3ccccc3c3ccccc32)c1 ; N#Cc1cccc(-c2ccc3c(c2)c2c4oc5ccccc5c4ccc2n3-c2ccccc2)c1-n1c2ccccc2c2ccccc21]

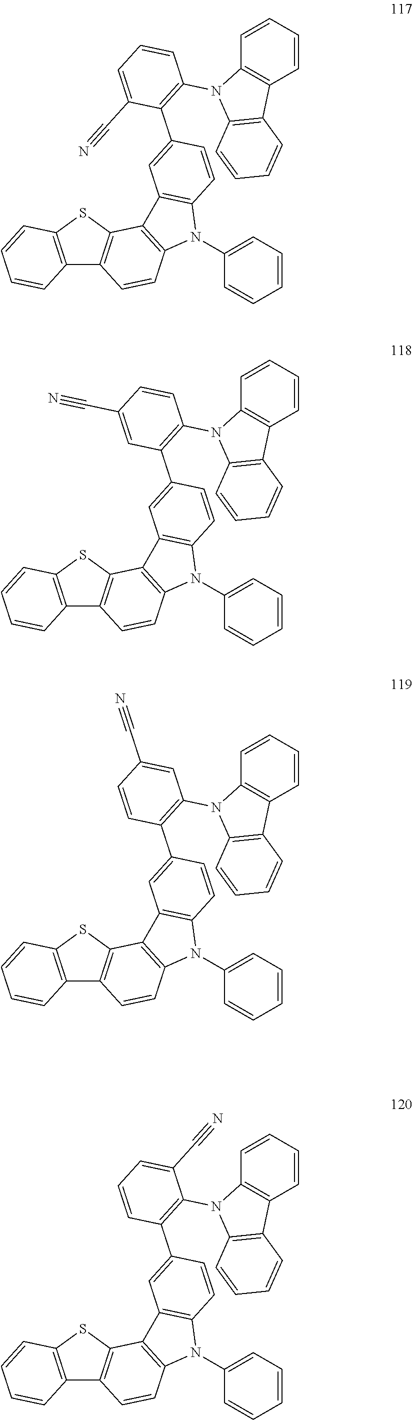 [SMILES: N#Cc1cccc(-n2c3ccccc3c3ccccc32)c1-c1ccc2c(c1)c1c3sc4ccccc4c3ccc1n2-c1ccccc1 ; N#Cc1ccc(-n2c3ccccc3c3ccccc32)c(-c2ccc3c(c2)c2c4sc5ccccc5c4ccc2n3-c2ccccc2)c1 ; N#Cc1ccc(-c2ccc3c(c2)c2c4sc5ccccc5c4ccc2n3-c2ccccc2)c(-n2c3ccccc3c3ccccc32)c1 ; N#Cc1cccc(-c2ccc3c(c2)c2c4sc5ccccc5c4ccc2n3-c2ccccc2)c1-n1c2ccccc2c2ccccc21]

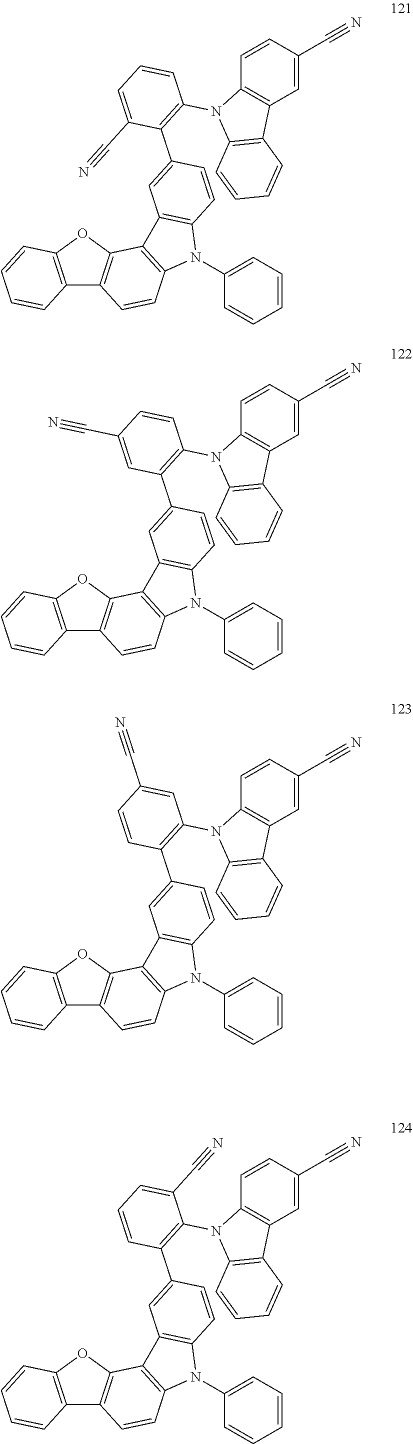 [SMILES: N#Cc1ccc2c(c1)c1ccccc1n2-c1cccc(C#N)c1-c1ccc2c(c1)c1c3oc4ccccc4c3ccc1n2-c1ccccc1 ; N#Cc1ccc(-n2c3ccccc3c3cc(C#N)ccc32)c(-c2ccc3c(c2)c2c4oc5ccccc5c4ccc2n3-c2ccccc2)c1 ; N#Cc1ccc(-c2ccc3c(c2)c2c4oc5ccccc5c4ccc2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1c3oc4ccccc4c3ccc1n2-c1ccccc1]

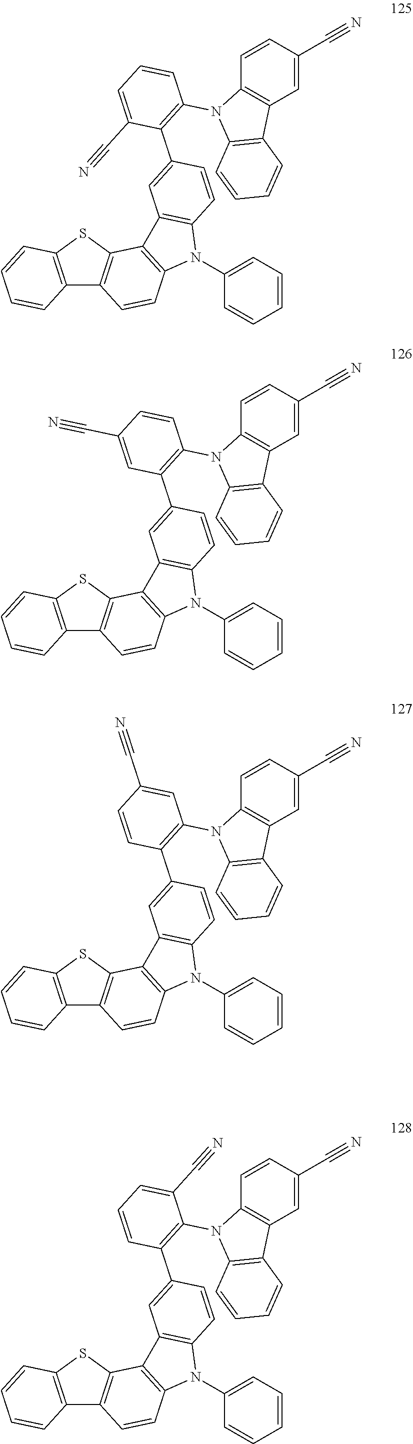 [SMILES: N#Cc1ccc2c(c1)c1ccccc1n2-c1cccc(C#N)c1-c1ccc2c(c1)c1c3sc4ccccc4c3ccc1n2-c1ccccc1 ; N#Cc1ccc(-n2c3ccccc3c3cc(C#N)ccc32)c(-c2ccc3c(c2)c2c4sc5ccccc5c4ccc2n3-c2ccccc2)c1 ; N#Cc1ccc(-c2ccc3c(c2)c2c4sc5ccccc5c4ccc2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1c3sc4ccccc4c3ccc1n2-c1ccccc1]

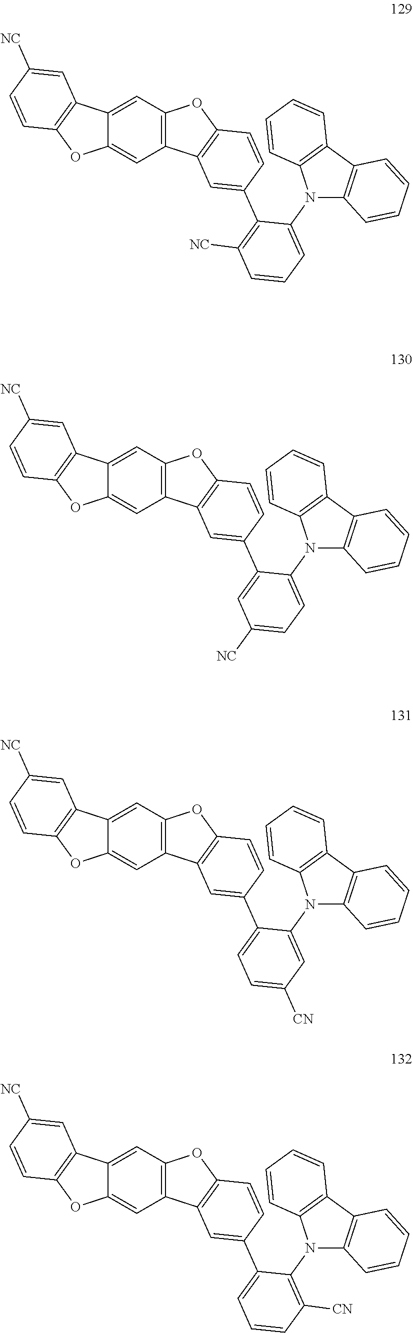 [SMILES: N#Cc1ccc2oc3cc4c(cc3c2c1)oc1ccc(-c2c(C#N)cccc2-n2c3ccccc3c3ccccc32)cc14 ; N#Cc1ccc(-n2c3ccccc3c3ccccc32)c(-c2ccc3oc4cc5c(cc4c3c2)oc2ccc(C#N)cc25)c1 ; N#Cc1ccc(-c2ccc3oc4cc5c(cc4c3c2)oc2ccc(C#N)cc25)c(-n2c3ccccc3c3ccccc32)c1 ; N#Cc1ccc2oc3cc4c(cc3c2c1)oc1ccc(-c2cccc(C#N)c2-n2c3ccccc3c3ccccc32)cc14]

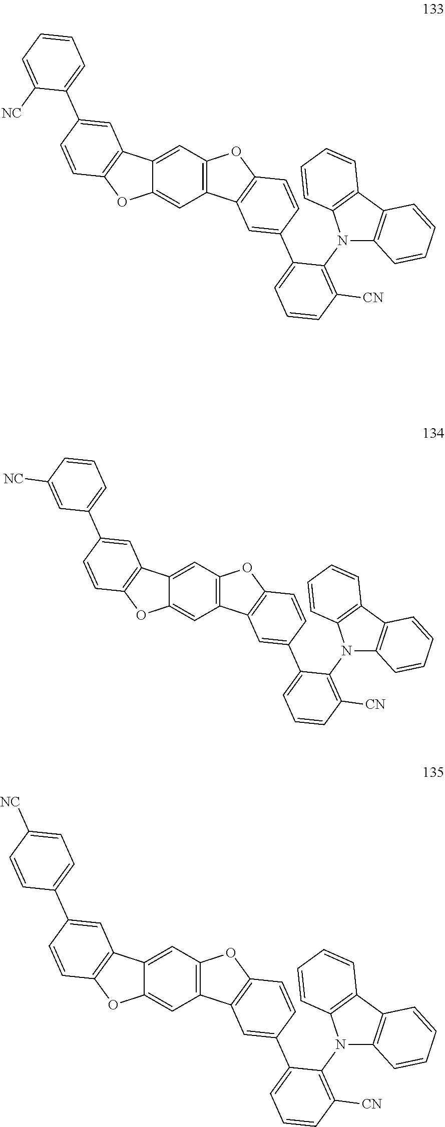 [SMILES: N#Cc1ccccc1-c1ccc2oc3cc4c(cc3c2c1)oc1ccc(-c2cccc(C#N)c2-n2c3ccccc3c3ccccc32)cc14 ; N#Cc1cccc(-c2ccc3oc4cc5c(cc4c3c2)oc2ccc(-c3cccc(C#N)c3-n3c4ccccc4c4ccccc43)cc25)c1 ; N#Cc1ccc(-c2ccc3oc4cc5c(cc4c3c2)oc2ccc(-c3cccc(C#N)c3-n3c4ccccc4c4ccccc43)cc25)cc1]

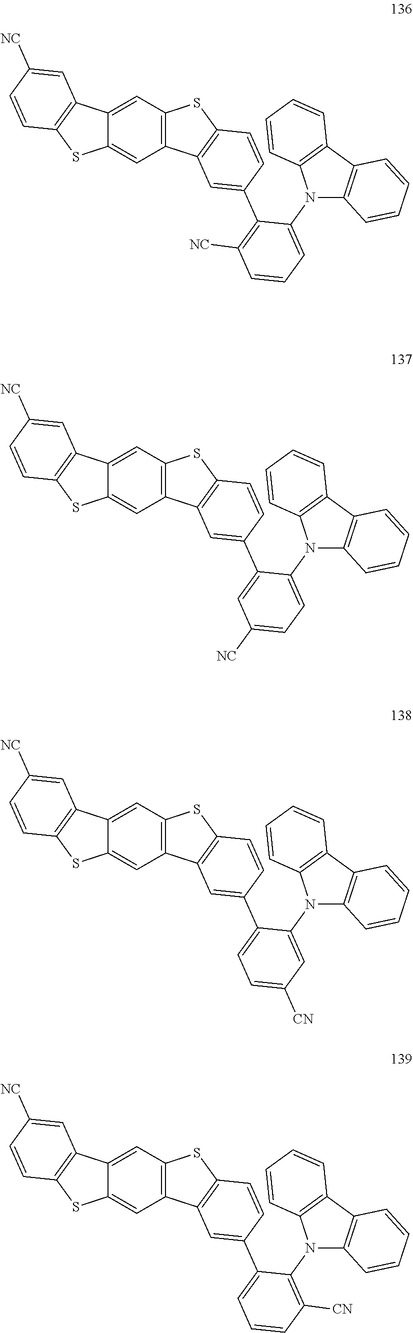 [SMILES: N#Cc1ccc2sc3cc4c(cc3c2c1)sc1ccc(-c2c(C#N)cccc2-n2c3ccccc3c3ccccc32)cc14 ; N#Cc1ccc(-n2c3ccccc3c3ccccc32)c(-c2ccc3sc4cc5c(cc4c3c2)sc2ccc(C#N)cc25)c1 ; N#Cc1ccc(-c2ccc3sc4cc5c(cc4c3c2)sc2ccc(C#N)cc25)c(-n2c3ccccc3c3ccccc32)c1 ; N#Cc1ccc2sc3cc4c(cc3c2c1)sc1ccc(-c2cccc(C#N)c2-n2c3ccccc3c3ccccc32)cc14]

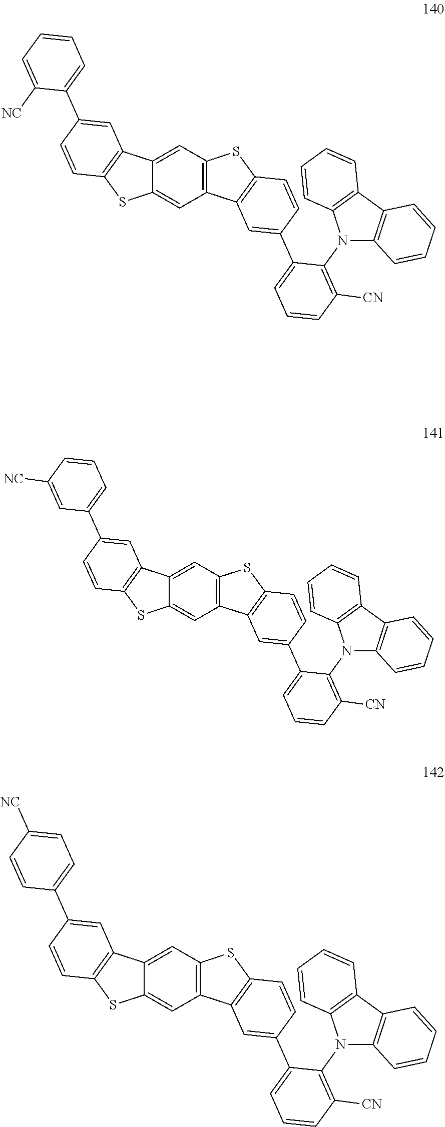 [SMILES: N#Cc1ccccc1-c1ccc2sc3cc4c(cc3c2c1)sc1ccc(-c2cccc(C#N)c2-n2c3ccccc3c3ccccc32)cc14 ; N#Cc1cccc(-c2ccc3sc4cc5c(cc4c3c2)sc2ccc(-c3cccc(C#N)c3-n3c4ccccc4c4ccccc43)cc25)c1 ; N#Cc1ccc(-c2ccc3sc4cc5c(cc4c3c2)sc2ccc(-c3cccc(C#N)c3-n3c4ccccc4c4ccccc43)cc25)cc1]

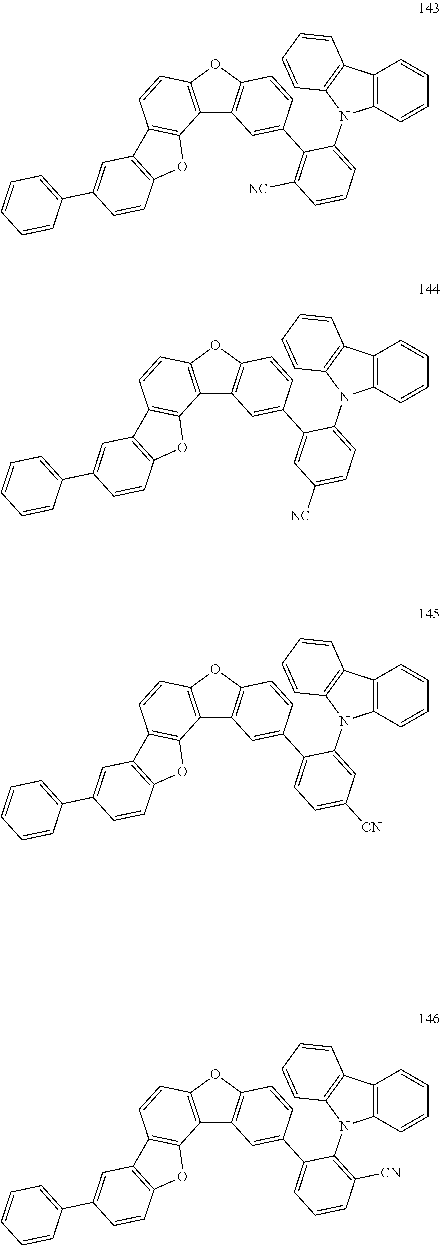 [SMILES: N#Cc1cccc(-n2c3ccccc3c3ccccc32)c1-c1ccc2oc3ccc4c5cc(-c6ccccc6)ccc5oc4c3c2c1 ; N#Cc1ccc(-n2c3ccccc3c3ccccc32)c(-c2ccc3oc4ccc5c6cc(-c7ccccc7)ccc6oc5c4c3c2)c1 ; N#Cc1ccc(-c2ccc3oc4ccc5c6cc(-c7ccccc7)ccc6oc5c4c3c2)c(-n2c3ccccc3c3ccccc32)c1 ; N#Cc1cccc(-c2ccc3oc4ccc5c6cc(-c7ccccc7)ccc6oc5c4c3c2)c1-n1c2ccccc2c2ccccc21]

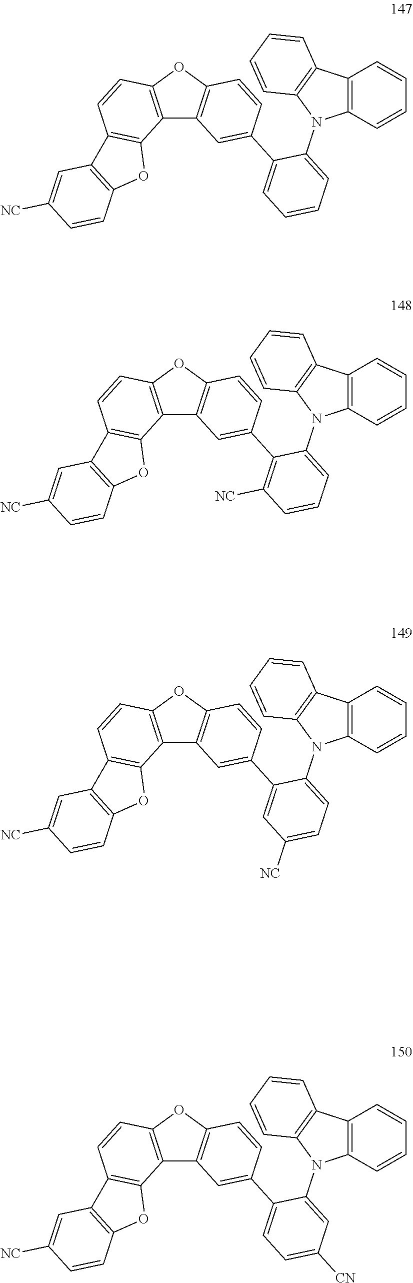 [SMILES: N#Cc1ccc2oc3c(ccc4oc5ccc(-c6ccccc6-n6c7ccccc7c7ccccc76)cc5c43)c2c1 ; N#Cc1ccc2oc3c(ccc4oc5ccc(-c6c(C#N)cccc6-n6c7ccccc7c7ccccc76)cc5c43)c2c1 ; N#Cc1ccc(-n2c3ccccc3c3ccccc32)c(-c2ccc3oc4ccc5c6cc(C#N)ccc6oc5c4c3c2)c1 ; N#Cc1ccc(-c2ccc3oc4ccc5c6cc(C#N)ccc6oc5c4c3c2)c(-n2c3ccccc3c3ccccc32)c1]

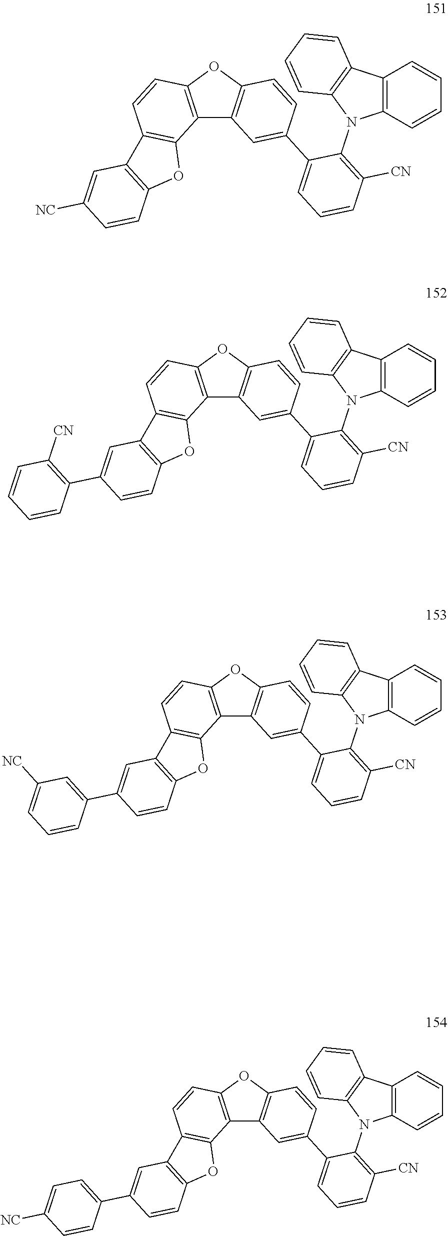 [SMILES: N#Cc1ccc2oc3c(ccc4oc5ccc(-c6cccc(C#N)c6-n6c7ccccc7c7ccccc76)cc5c43)c2c1 ; N#Cc1ccccc1-c1ccc2oc3c(ccc4oc5ccc(-c6cccc(C#N)c6-n6c7ccccc7c7ccccc76)cc5c43)c2c1 ; N#Cc1cccc(-c2ccc3oc4c(ccc5oc6ccc(-c7cccc(C#N)c7-n7c8ccccc8c8ccccc87)cc6c54)c3c2)c1 ; N#Cc1ccc(-c2ccc3oc4c(ccc5oc6ccc(-c7cccc(C#N)c7-n7c8ccccc8c8ccccc87)cc6c54)c3c2)cc1]

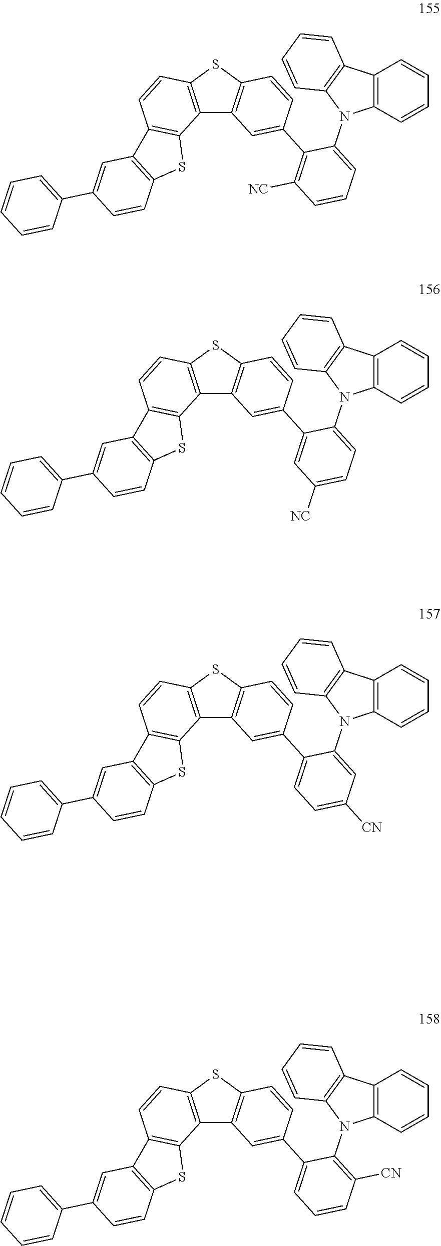 [SMILES: N#Cc1cccc(-n2c3ccccc3c3ccccc32)c1-c1ccc2sc3ccc4c5cc(-c6ccccc6)ccc5sc4c3c2c1 ; N#Cc1ccc(-n2c3ccccc3c3ccccc32)c(-c2ccc3sc4ccc5c6cc(-c7ccccc7)ccc6sc5c4c3c2)c1 ; N#Cc1ccc(-c2ccc3sc4ccc5c6cc(-c7ccccc7)ccc6sc5c4c3c2)c(-n2c3ccccc3c3ccccc32)c1 ; N#Cc1cccc(-c2ccc3sc4ccc5c6cc(-c7ccccc7)ccc6sc5c4c3c2)c1-n1c2ccccc2c2ccccc21]

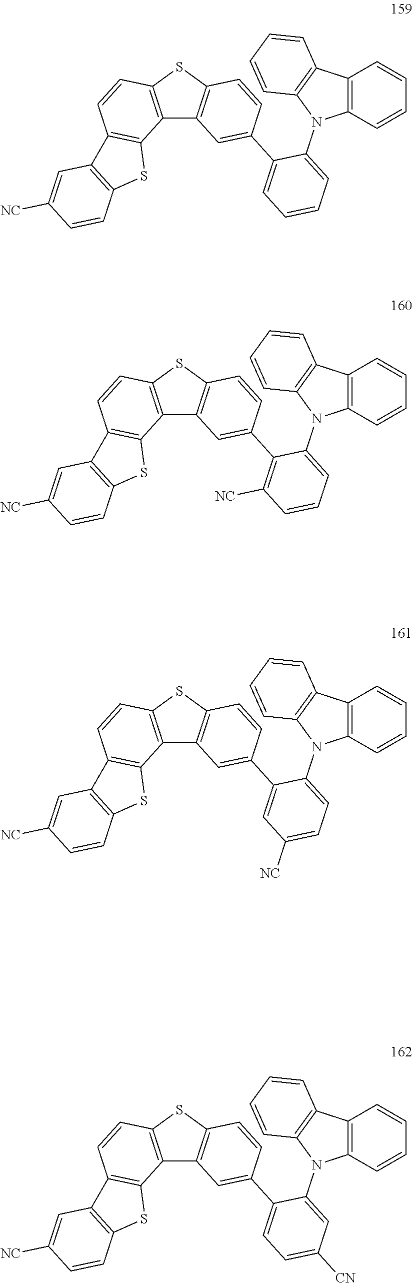 [SMILES: N#Cc1ccc2sc3c(ccc4sc5ccc(-c6ccccc6-n6c7ccccc7c7ccccc76)cc5c43)c2c1 ; N#Cc1ccc2sc3c(ccc4sc5ccc(-c6c(C#N)cccc6-n6c7ccccc7c7ccccc76)cc5c43)c2c1 ; N#Cc1ccc(-n2c3ccccc3c3ccccc32)c(-c2ccc3sc4ccc5c6cc(C#N)ccc6sc5c4c3c2)c1 ; N#Cc1ccc(-c2ccc3sc4ccc5c6cc(C#N)ccc6sc5c4c3c2)c(-n2c3ccccc3c3ccccc32)c1]

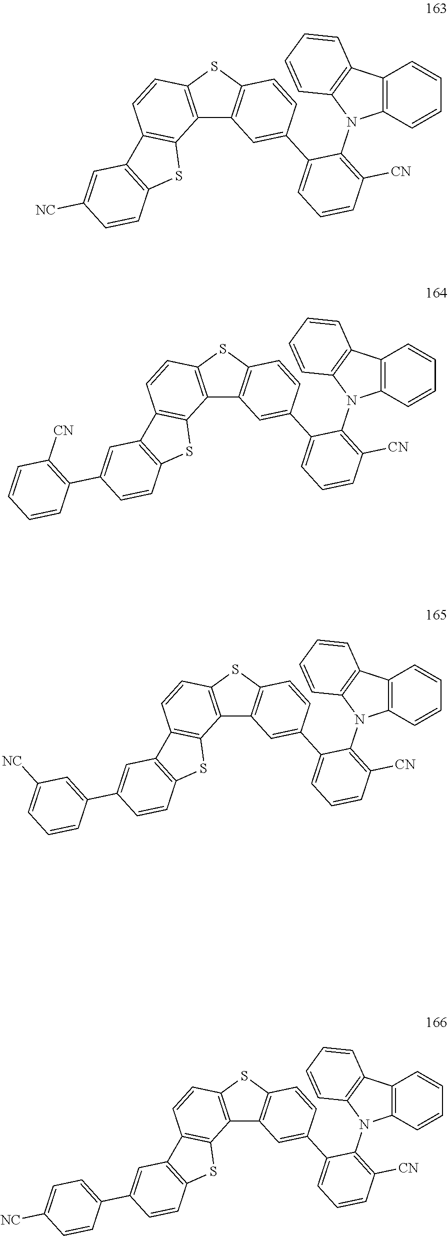 [SMILES: N#Cc1ccc2sc3c(ccc4sc5ccc(-c6cccc(C#N)c6-n6c7ccccc7c7ccccc76)cc5c43)c2c1 ; N#Cc1ccccc1-c1ccc2sc3c(ccc4sc5ccc(-c6cccc(C#N)c6-n6c7ccccc7c7ccccc76)cc5c43)c2c1 ; N#Cc1cccc(-c2ccc3sc4c(ccc5sc6ccc(-c7cccc(C#N)c7-n7c8ccccc8c8ccccc87)cc6c54)c3c2)c1 ; N#Cc1ccc(-c2ccc3sc4c(ccc5sc6ccc(-c7cccc(C#N)c7-n7c8ccccc8c8ccccc87)cc6c54)c3c2)cc1]

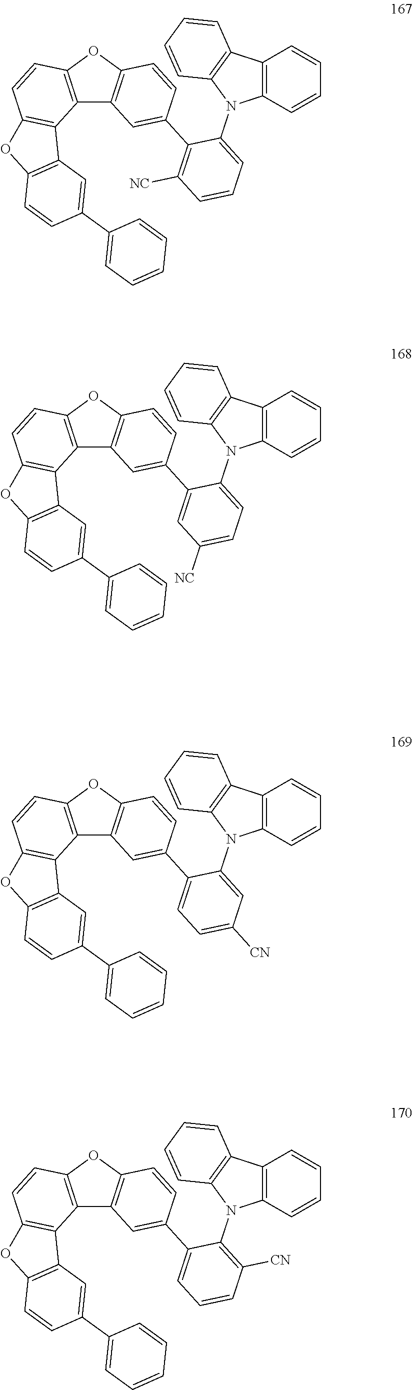 [SMILES: N#Cc1cccc(-n2c3ccccc3c3ccccc32)c1-c1ccc2oc3ccc4oc5ccc(-c6ccccc6)cc5c4c3c2c1 ; N#Cc1ccc(-n2c3ccccc3c3ccccc32)c(-c2ccc3oc4ccc5oc6ccc(-c7ccccc7)cc6c5c4c3c2)c1 ; N#Cc1ccc(-c2ccc3oc4ccc5oc6ccc(-c7ccccc7)cc6c5c4c3c2)c(-n2c3ccccc3c3ccccc32)c1 ; N#Cc1cccc(-c2ccc3oc4ccc5oc6ccc(-c7ccccc7)cc6c5c4c3c2)c1-n1c2ccccc2c2ccccc21]

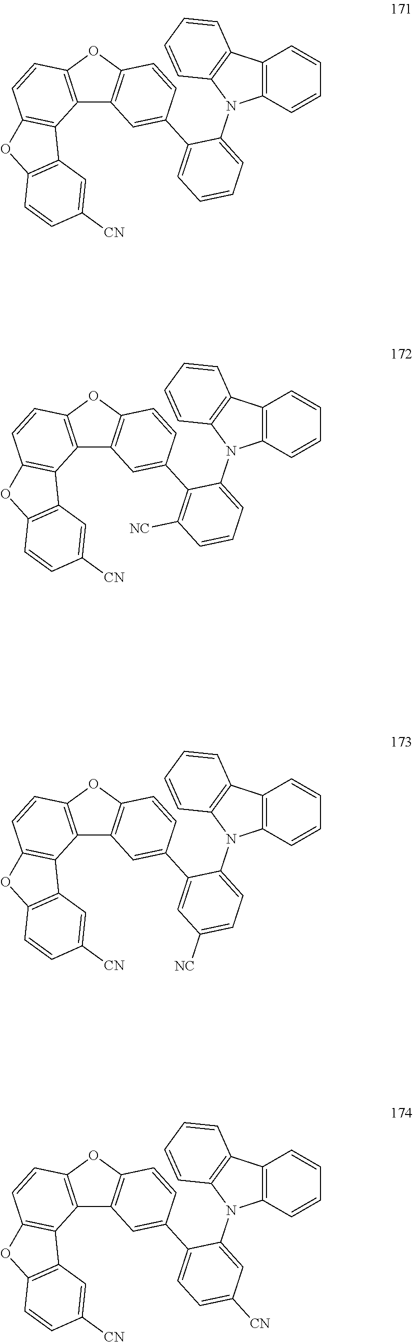 [SMILES: N#Cc1ccc2oc3ccc4oc5ccc(-c6ccccc6-n6c7ccccc7c7ccccc76)cc5c4c3c2c1 ; N#Cc1ccc2oc3ccc4oc5ccc(-c6c(C#N)cccc6-n6c7ccccc7c7ccccc76)cc5c4c3c2c1 ; N#Cc1ccc(-n2c3ccccc3c3ccccc32)c(-c2ccc3oc4ccc5oc6ccc(C#N)cc6c5c4c3c2)c1 ; N#Cc1ccc(-c2ccc3oc4ccc5oc6ccc(C#N)cc6c5c4c3c2)c(-n2c3ccccc3c3ccccc32)c1]

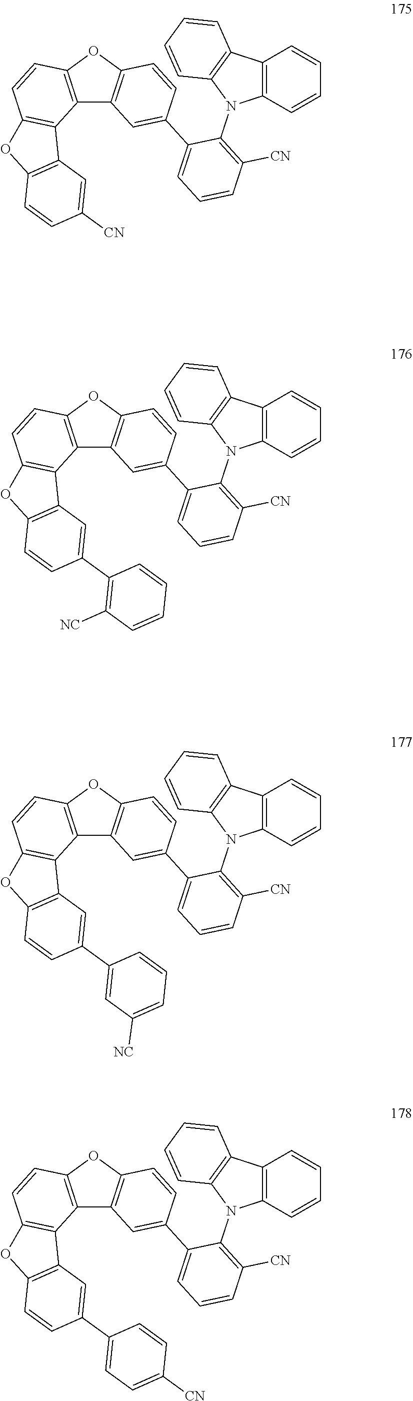 [SMILES: N#Cc1ccc2oc3ccc4oc5ccc(-c6cccc(C#N)c6-n6c7ccccc7c7ccccc76)cc5c4c3c2c1 ; N#Cc1ccccc1-c1ccc2oc3ccc4oc5ccc(-c6cccc(C#N)c6-n6c7ccccc7c7ccccc76)cc5c4c3c2c1 ; N#Cc1cccc(-c2ccc3oc4ccc5oc6ccc(-c7cccc(C#N)c7-n7c8ccccc8c8ccccc87)cc6c5c4c3c2)c1 ; N#Cc1ccc(-c2ccc3oc4ccc5oc6ccc(-c7cccc(C#N)c7-n7c8ccccc8c8ccccc87)cc6c5c4c3c2)cc1]

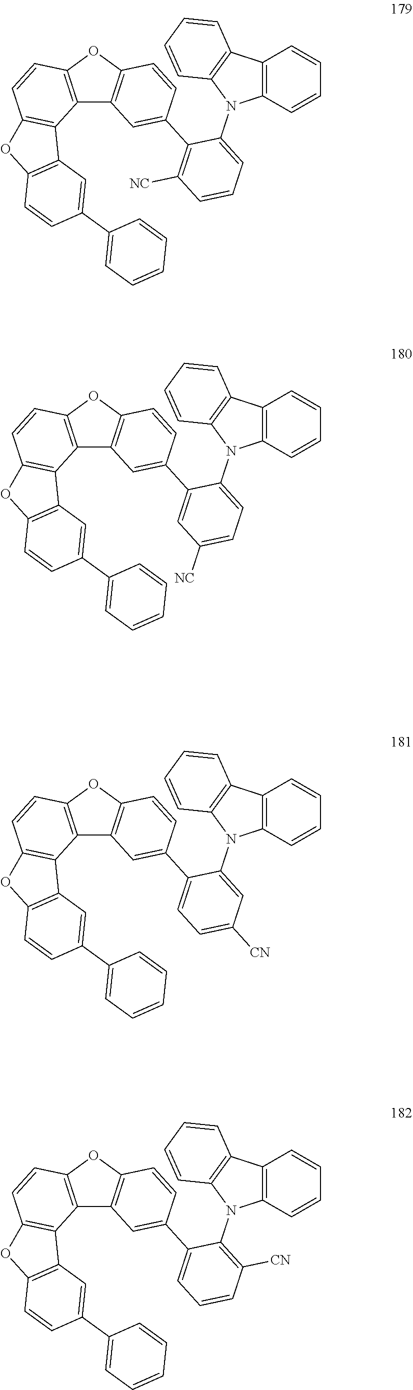 [SMILES: N#Cc1cccc(-n2c3ccccc3c3ccccc32)c1-c1ccc2oc3ccc4oc5ccc(-c6ccccc6)cc5c4c3c2c1 ; N#Cc1ccc(-n2c3ccccc3c3ccccc32)c(-c2ccc3oc4ccc5oc6ccc(-c7ccccc7)cc6c5c4c3c2)c1 ; N#Cc1ccc(-c2ccc3oc4ccc5oc6ccc(-c7ccccc7)cc6c5c4c3c2)c(-n2c3ccccc3c3ccccc32)c1 ; N#Cc1cccc(-c2ccc3oc4ccc5oc6ccc(-c7ccccc7)cc6c5c4c3c2)c1-n1c2ccccc2c2ccccc21]

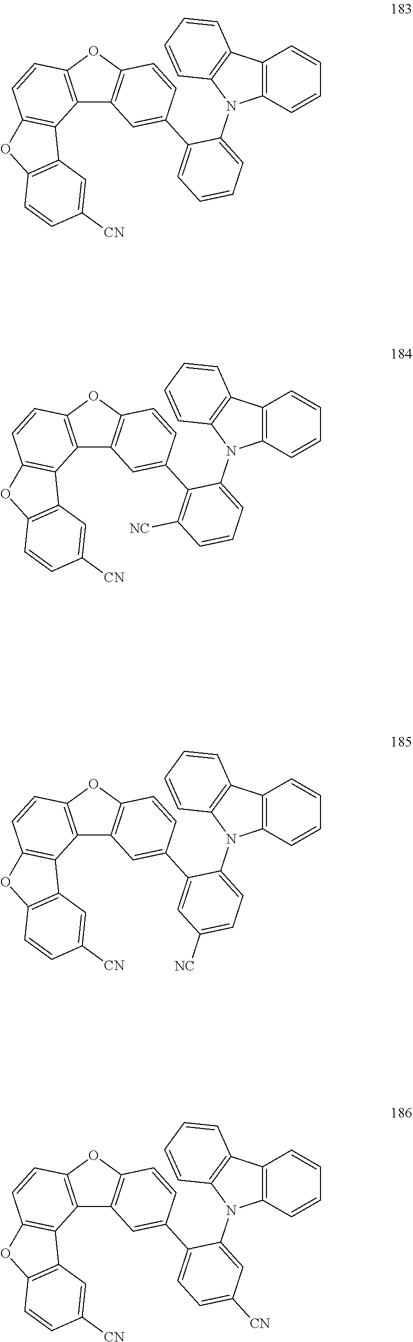 [SMILES: N#Cc1ccc2oc3ccc4oc5ccc(-c6ccccc6-n6c7ccccc7c7ccccc76)cc5c4c3c2c1 ; N#Cc1ccc2oc3ccc4oc5ccc(-c6c(C#N)cccc6-n6c7ccccc7c7ccccc76)cc5c4c3c2c1 ; N#Cc1ccc(-n2c3ccccc3c3ccccc32)c(-c2ccc3oc4ccc5oc6ccc(C#N)cc6c5c4c3c2)c1 ; N#Cc1ccc(-c2ccc3oc4ccc5oc6ccc(C#N)cc6c5c4c3c2)c(-n2c3ccccc3c3ccccc32)c1]

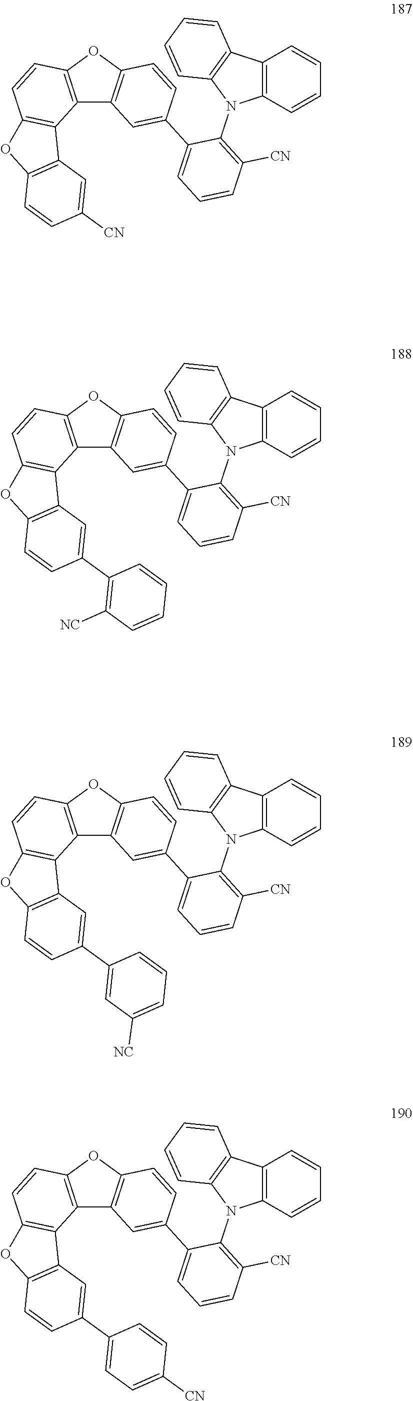 [SMILES: N#Cc1ccc2oc3ccc4oc5ccc(-c6cccc(C#N)c6-n6c7ccccc7c7ccccc76)cc5c4c3c2c1 ; N#Cc1ccccc1-c1ccc2oc3ccc4oc5ccc(-c6cccc(C#N)c6-n6c7ccccc7c7ccccc76)cc5c4c3c2c1 ; N#Cc1cccc(-c2ccc3oc4ccc5oc6ccc(-c7cccc(C#N)c7-n7c8ccccc8c8ccccc87)cc6c5c4c3c2)c1 ; N#Cc1ccc(-c2ccc3oc4ccc5oc6ccc(-c7cccc(C#N)c7-n7c8ccccc8c8ccccc87)cc6c5c4c3c2)cc1]

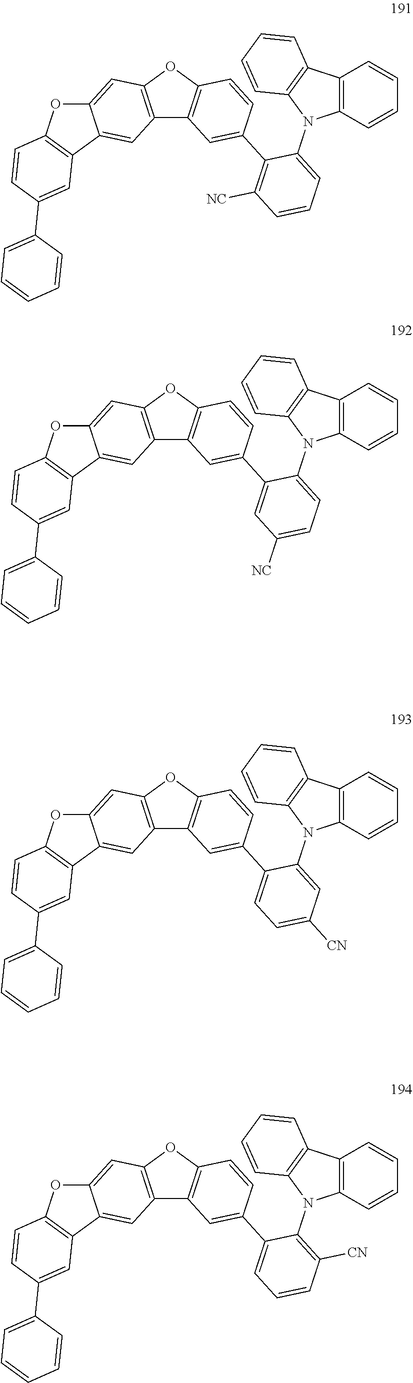 [SMILES: N#Cc1cccc(-n2c3ccccc3c3ccccc32)c1-c1ccc2oc3cc4oc5ccc(-c6ccccc6)cc5c4cc3c2c1 ; N#Cc1ccc(-n2c3ccccc3c3ccccc32)c(-c2ccc3oc4cc5oc6ccc(-c7ccccc7)cc6c5cc4c3c2)c1 ; N#Cc1ccc(-c2ccc3oc4cc5oc6ccc(-c7ccccc7)cc6c5cc4c3c2)c(-n2c3ccccc3c3ccccc32)c1 ; N#Cc1cccc(-c2ccc3oc4cc5oc6ccc(-c7ccccc7)cc6c5cc4c3c2)c1-n1c2ccccc2c2ccccc21]

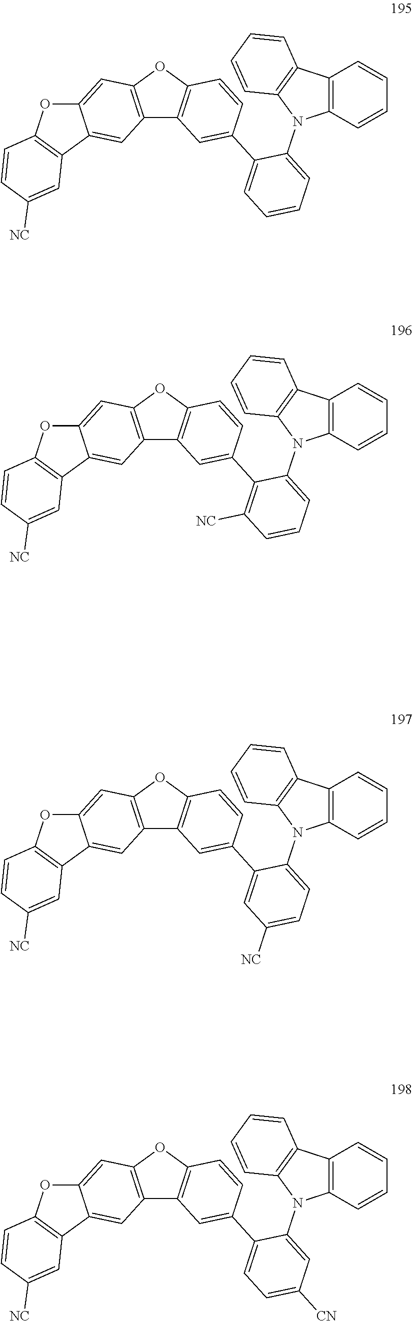 [SMILES: N#Cc1ccc2oc3cc4oc5ccc(-c6ccccc6-n6c7ccccc7c7ccccc76)cc5c4cc3c2c1 ; N#Cc1ccc2oc3cc4oc5ccc(-c6c(C#N)cccc6-n6c7ccccc7c7ccccc76)cc5c4cc3c2c1 ; N#Cc1ccc(-n2c3ccccc3c3ccccc32)c(-c2ccc3oc4cc5oc6ccc(C#N)cc6c5cc4c3c2)c1 ; N#Cc1ccc(-c2ccc3oc4cc5oc6ccc(C#N)cc6c5cc4c3c2)c(-n2c3ccccc3c3ccccc32)c1]

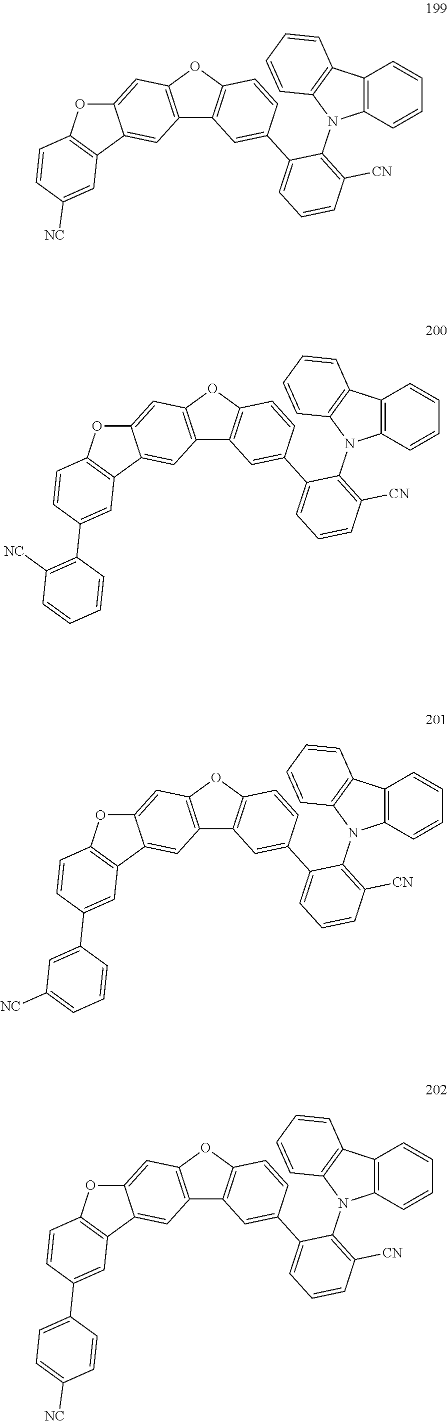 [SMILES: N#Cc1ccc2oc3cc4oc5ccc(-c6cccc(C#N)c6-n6c7ccccc7c7ccccc76)cc5c4cc3c2c1 ; N#Cc1ccccc1-c1ccc2oc3cc4oc5ccc(-c6cccc(C#N)c6-n6c7ccccc7c7ccccc76)cc5c4cc3c2c1 ; N#Cc1cccc(-c2ccc3oc4cc5oc6ccc(-c7cccc(C#N)c7-n7c8ccccc8c8ccccc87)cc6c5cc4c3c2)c1 ; N#Cc1ccc(-c2ccc3oc4cc5oc6ccc(-c7cccc(C#N)c7-n7c8ccccc8c8ccccc87)cc6c5cc4c3c2)cc1]

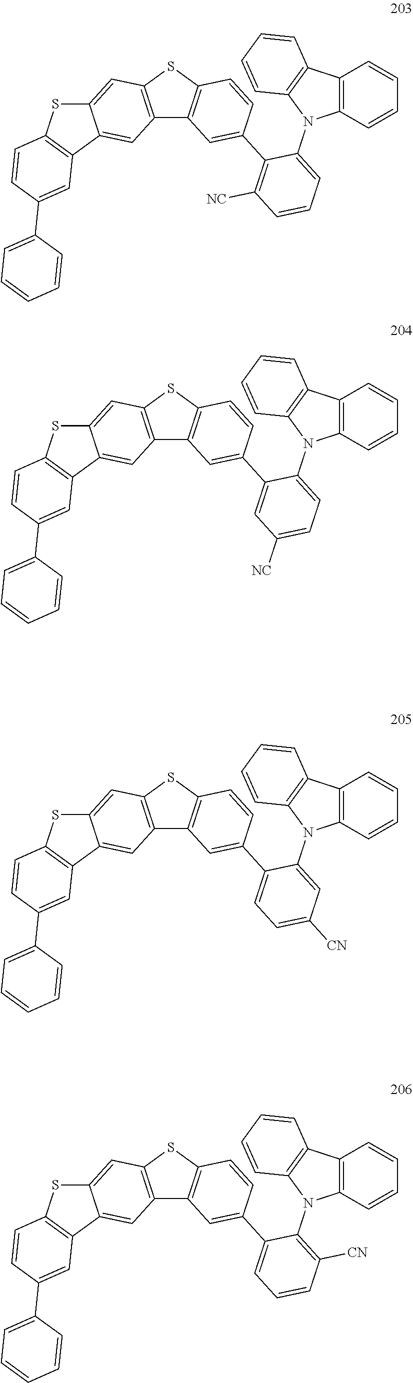 [SMILES: N#Cc1cccc(-n2c3ccccc3c3ccccc32)c1-c1ccc2sc3cc4sc5ccc(-c6ccccc6)cc5c4cc3c2c1 ; N#Cc1ccc(-n2c3ccccc3c3ccccc32)c(-c2ccc3sc4cc5sc6ccc(-c7ccccc7)cc6c5cc4c3c2)c1 ; N#Cc1ccc(-c2ccc3sc4cc5sc6ccc(-c7ccccc7)cc6c5cc4c3c2)c(-n2c3ccccc3c3ccccc32)c1 ; N#Cc1cccc(-c2ccc3sc4cc5sc6ccc(-c7ccccc7)cc6c5cc4c3c2)c1-n1c2ccccc2c2ccccc21]

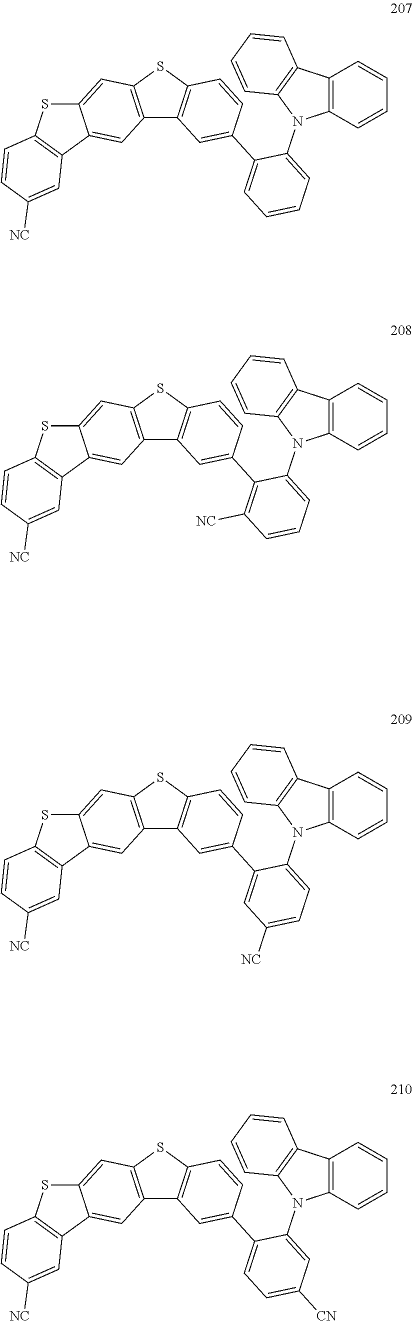 [SMILES: N#Cc1ccc2sc3cc4sc5ccc(-c6ccccc6-n6c7ccccc7c7ccccc76)cc5c4cc3c2c1 ; N#Cc1ccc2sc3cc4sc5ccc(-c6c(C#N)cccc6-n6c7ccccc7c7ccccc76)cc5c4cc3c2c1 ; N#Cc1ccc(-n2c3ccccc3c3ccccc32)c(-c2ccc3sc4cc5sc6ccc(C#N)cc6c5cc4c3c2)c1 ; N#Cc1ccc(-c2ccc3sc4cc5sc6ccc(C#N)cc6c5cc4c3c2)c(-n2c3ccccc3c3ccccc32)c1]

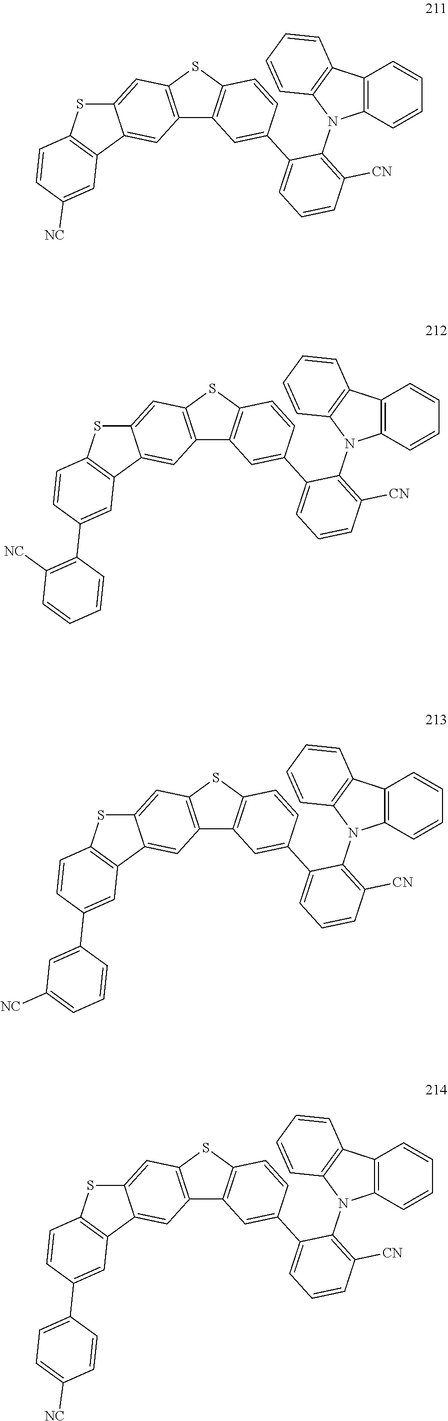 [SMILES: N#Cc1ccc2sc3cc4sc5ccc(-c6cccc(C#N)c6-n6c7ccccc7c7ccccc76)cc5c4cc3c2c1 ; N#Cc1ccccc1-c1ccc2sc3cc4sc5ccc(-c6cccc(C#N)c6-n6c7ccccc7c7ccccc76)cc5c4cc3c2c1 ; N#Cc1cccc(-c2ccc3sc4cc5sc6ccc(-c7cccc(C#N)c7-n7c8ccccc8c8ccccc87)cc6c5cc4c3c2)c1 ; N#Cc1ccc(-c2ccc3sc4cc5sc6ccc(-c7cccc(C#N)c7-n7c8ccccc8c8ccccc87)cc6c5cc4c3c2)cc1]

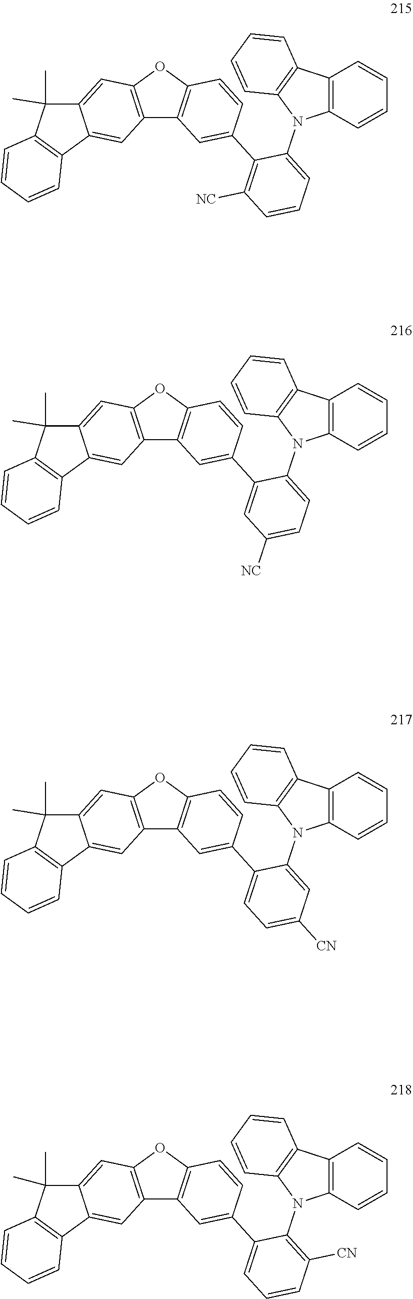 [SMILES: CC1(C)c2ccccc2-c2cc3c(cc21)oc1ccc(-c2c(C#N)cccc2-n2c4ccccc4c4ccccc42)cc13 ; CC1(C)c2ccccc2-c2cc3c(cc21)oc1ccc(-c2cc(C#N)ccc2-n2c4ccccc4c4ccccc42)cc13 ; CC1(C)c2ccccc2-c2cc3c(cc21)oc1ccc(-c2ccc(C#N)cc2-n2c4ccccc4c4ccccc42)cc13 ; CC1(C)c2ccccc2-c2cc3c(cc21)oc1ccc(-c2cccc(C#N)c2-n2c4ccccc4c4ccccc42)cc13]

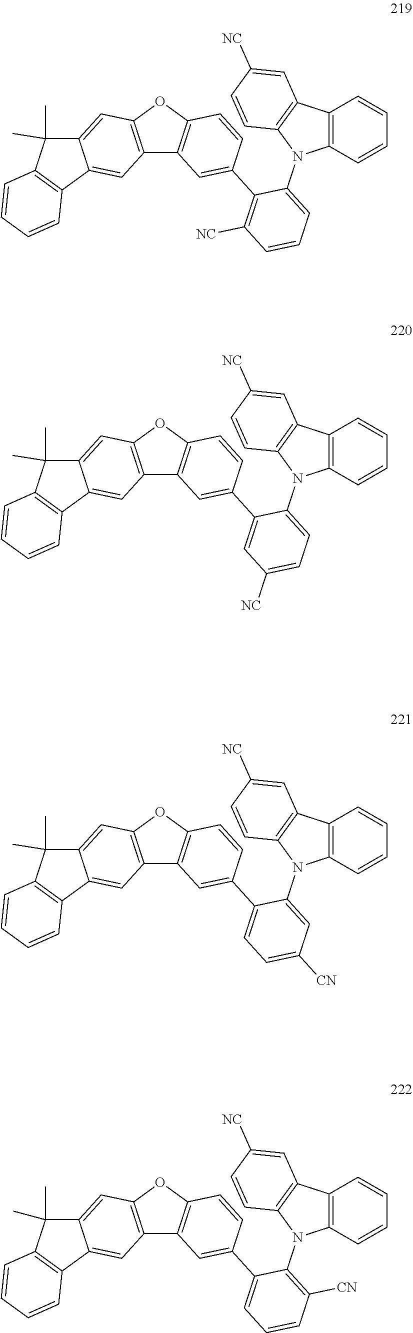 [SMILES: CC1(C)c2ccccc2-c2cc3c(cc21)oc1ccc(-c2c(C#N)cccc2-n2c4ccccc4c4cc(C#N)ccc42)cc13 ; CC1(C)c2ccccc2-c2cc3c(cc21)oc1ccc(-c2cc(C#N)ccc2-n2c4ccccc4c4cc(C#N)ccc42)cc13 ; CC1(C)c2ccccc2-c2cc3c(cc21)oc1ccc(-c2ccc(C#N)cc2-n2c4ccccc4c4cc(C#N)ccc42)cc13 ; CC1(C)c2ccccc2-c2cc3c(cc21)oc1ccc(-c2cccc(C#N)c2-n2c4ccccc4c4cc(C#N)ccc42)cc13]

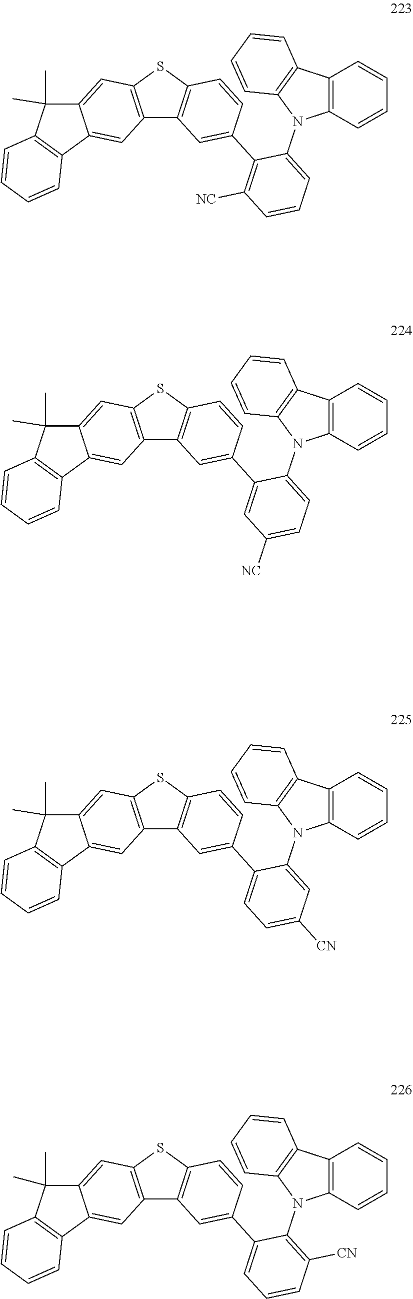 [SMILES: CC1(C)c2ccccc2-c2cc3c(cc21)sc1ccc(-c2c(C#N)cccc2-n2c4ccccc4c4ccccc42)cc13 ; CC1(C)c2ccccc2-c2cc3c(cc21)sc1ccc(-c2cc(C#N)ccc2-n2c4ccccc4c4ccccc42)cc13 ; CC1(C)c2ccccc2-c2cc3c(cc21)sc1ccc(-c2ccc(C#N)cc2-n2c4ccccc4c4ccccc42)cc13 ; CC1(C)c2ccccc2-c2cc3c(cc21)sc1ccc(-c2cccc(C#N)c2-n2c4ccccc4c4ccccc42)cc13]

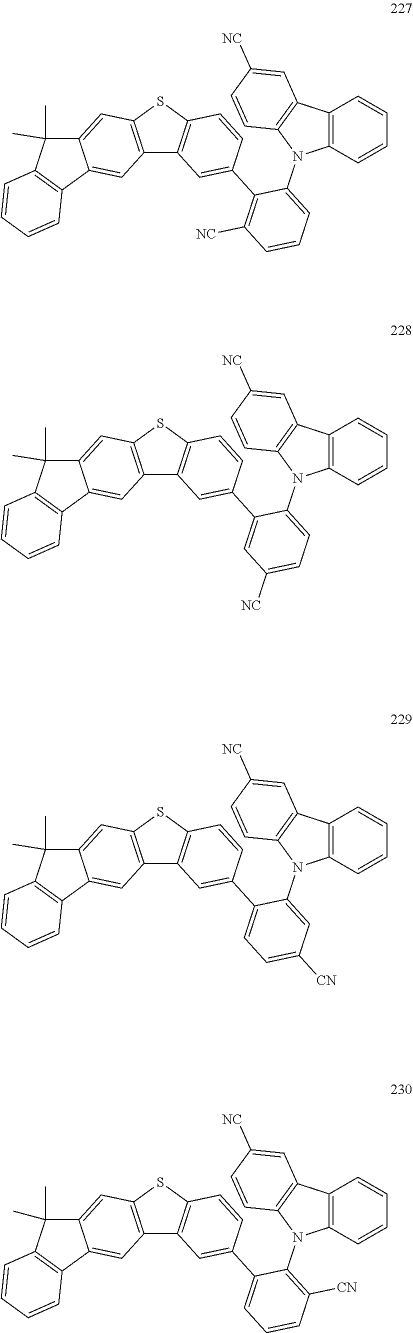 [SMILES: CC1(C)c2ccccc2-c2cc3c(cc21)sc1ccc(-c2c(C#N)cccc2-n2c4ccccc4c4cc(C#N)ccc42)cc13 ; CC1(C)c2ccccc2-c2cc3c(cc21)sc1ccc(-c2cc(C#N)ccc2-n2c4ccccc4c4cc(C#N)ccc42)cc13 ; CC1(C)c2ccccc2-c2cc3c(cc21)sc1ccc(-c2ccc(C#N)cc2-n2c4ccccc4c4cc(C#N)ccc42)cc13 ; CC1(C)c2ccccc2-c2cc3c(cc21)sc1ccc(-c2cccc(C#N)c2-n2c4ccccc4c4cc(C#N)ccc42)cc13]

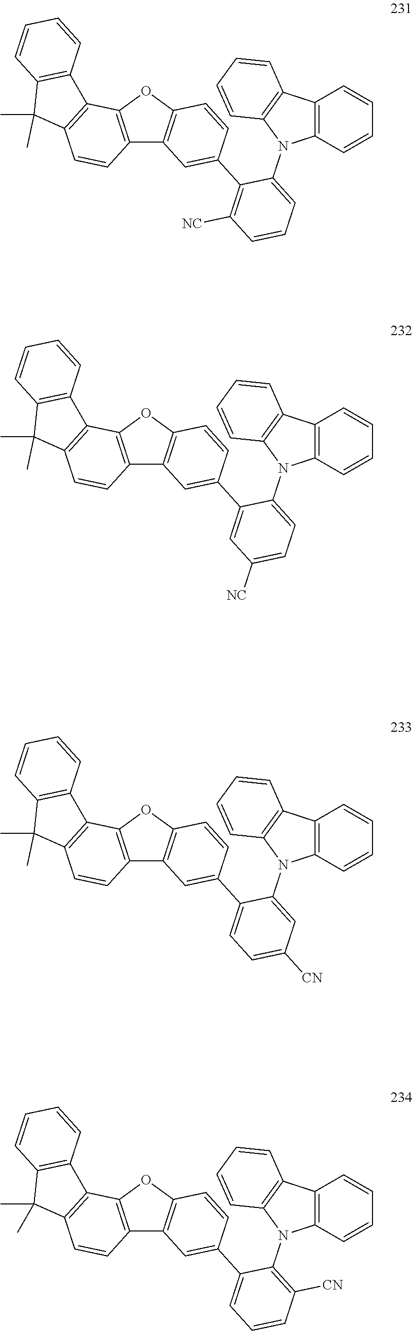 [SMILES: CC1(C)c2ccccc2-c2c1ccc1c2oc2ccc(-c3c(C#N)cccc3-n3c4ccccc4c4ccccc43)cc21 ; CC1(C)c2ccccc2-c2c1ccc1c2oc2ccc(-c3cc(C#N)ccc3-n3c4ccccc4c4ccccc43)cc21 ; CC1(C)c2ccccc2-c2c1ccc1c2oc2ccc(-c3ccc(C#N)cc3-n3c4ccccc4c4ccccc43)cc21 ; CC1(C)c2ccccc2-c2c1ccc1c2oc2ccc(-c3cccc(C#N)c3-n3c4ccccc4c4ccccc43)cc21]

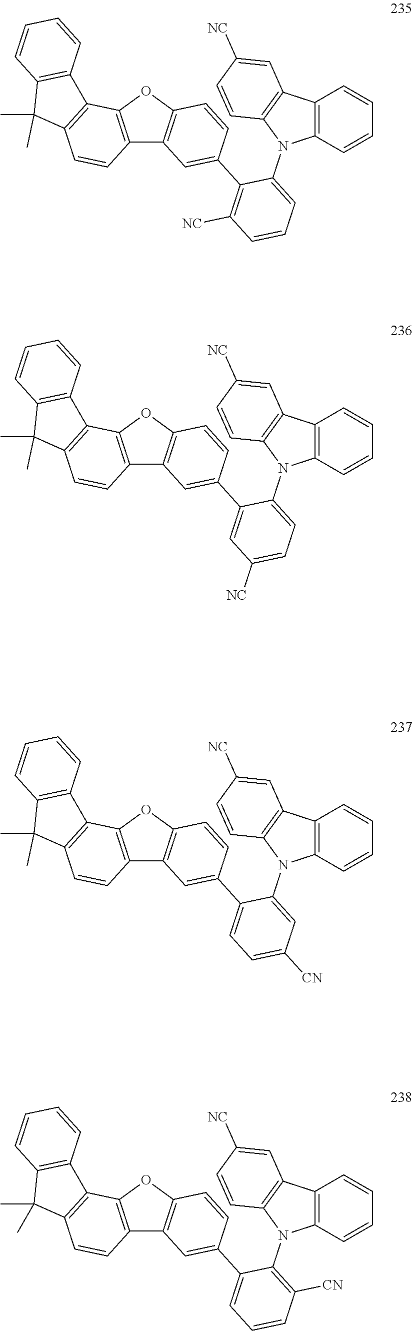 [SMILES: CC1(C)c2ccccc2-c2c1ccc1c2oc2ccc(-c3c(C#N)cccc3-n3c4ccccc4c4cc(C#N)ccc43)cc21 ; CC1(C)c2ccccc2-c2c1ccc1c2oc2ccc(-c3cc(C#N)ccc3-n3c4ccccc4c4cc(C#N)ccc43)cc21 ; CC1(C)c2ccccc2-c2c1ccc1c2oc2ccc(-c3ccc(C#N)cc3-n3c4ccccc4c4cc(C#N)ccc43)cc21 ; CC1(C)c2ccccc2-c2c1ccc1c2oc2ccc(-c3cccc(C#N)c3-n3c4ccccc4c4cc(C#N)ccc43)cc21]

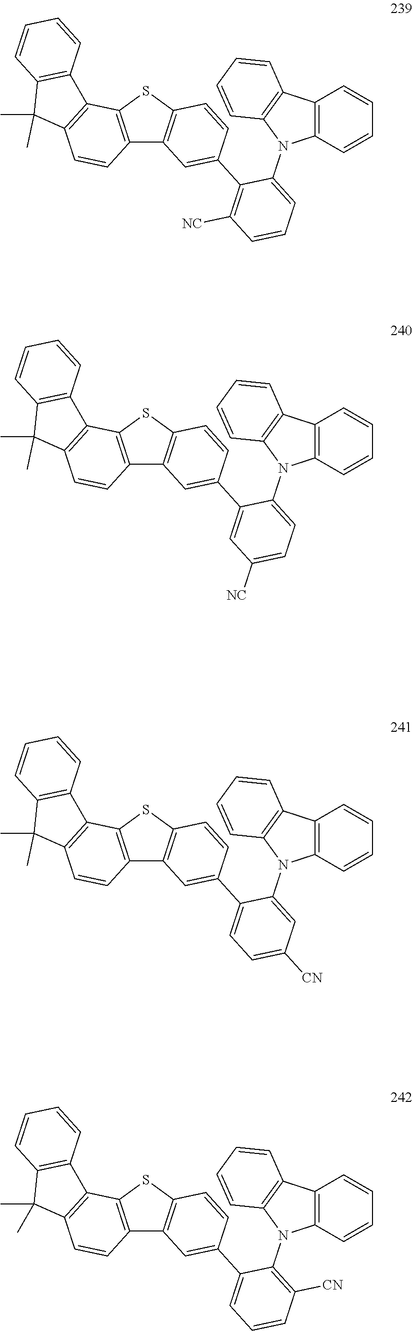 [SMILES: CC1(C)c2ccccc2-c2c1ccc1c2sc2ccc(-c3c(C#N)cccc3-n3c4ccccc4c4ccccc43)cc21 ; CC1(C)c2ccccc2-c2c1ccc1c2sc2ccc(-c3cc(C#N)ccc3-n3c4ccccc4c4ccccc43)cc21 ; CC1(C)c2ccccc2-c2c1ccc1c2sc2ccc(-c3ccc(C#N)cc3-n3c4ccccc4c4ccccc43)cc21 ; CC1(C)c2ccccc2-c2c1ccc1c2sc2ccc(-c3cccc(C#N)c3-n3c4ccccc4c4ccccc43)cc21]

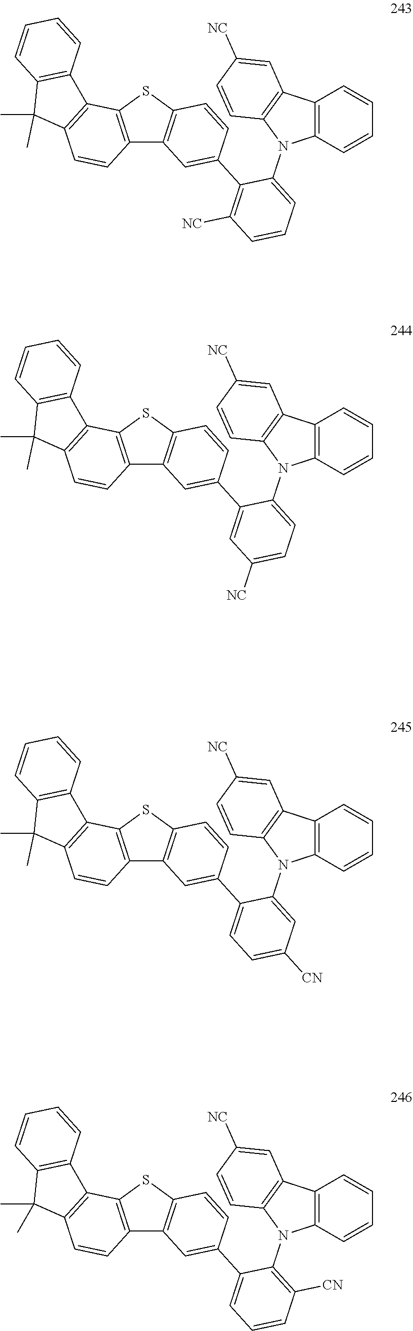 [SMILES: CC1(C)c2ccccc2-c2c1ccc1c2sc2ccc(-c3c(C#N)cccc3-n3c4ccccc4c4cc(C#N)ccc43)cc21 ; CC1(C)c2ccccc2-c2c1ccc1c2sc2ccc(-c3cc(C#N)ccc3-n3c4ccccc4c4cc(C#N)ccc43)cc21 ; CC1(C)c2ccccc2-c2c1ccc1c2sc2ccc(-c3ccc(C#N)cc3-n3c4ccccc4c4cc(C#N)ccc43)cc21 ; CC1(C)c2ccccc2-c2c1ccc1c2sc2ccc(-c3cccc(C#N)c3-n3c4ccccc4c4cc(C#N)ccc43)cc21]

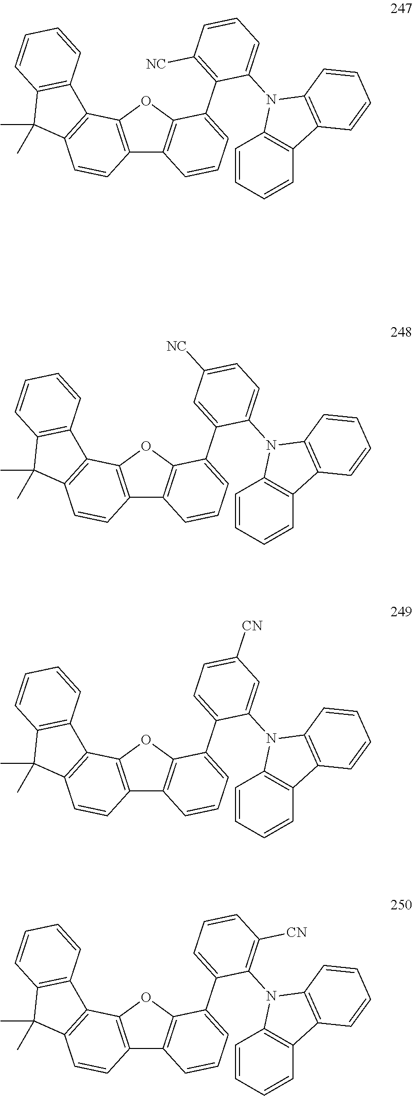 [SMILES: CC1(C)c2ccccc2-c2c1ccc1c2oc2c(-c3c(C#N)cccc3-n3c4ccccc4c4ccccc43)cccc21 ; CC1(C)c2ccccc2-c2c1ccc1c2oc2c(-c3cc(C#N)ccc3-n3c4ccccc4c4ccccc43)cccc21 ; CC1(C)c2ccccc2-c2c1ccc1c2oc2c(-c3ccc(C#N)cc3-n3c4ccccc4c4ccccc43)cccc21 ; CC1(C)c2ccccc2-c2c1ccc1c2oc2c(-c3cccc(C#N)c3-n3c4ccccc4c4ccccc43)cccc21]

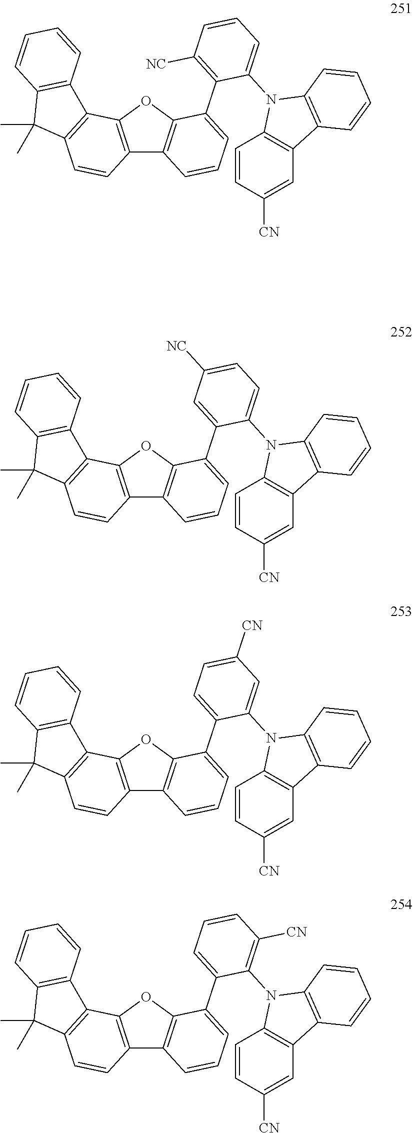 [SMILES: CC1(C)c2ccccc2-c2c1ccc1c2oc2c(-c3c(C#N)cccc3-n3c4ccccc4c4cc(C#N)ccc43)cccc21 ; CC1(C)c2ccccc2-c2c1ccc1c2oc2c(-c3cc(C#N)ccc3-n3c4ccccc4c4cc(C#N)ccc43)cccc21 ; CC1(C)c2ccccc2-c2c1ccc1c2oc2c(-c3ccc(C#N)cc3-n3c4ccccc4c4cc(C#N)ccc43)cccc21 ; CC1(C)c2ccccc2-c2c1ccc1c2oc2c(-c3cccc(C#N)c3-n3c4ccccc4c4cc(C#N)ccc43)cccc21]

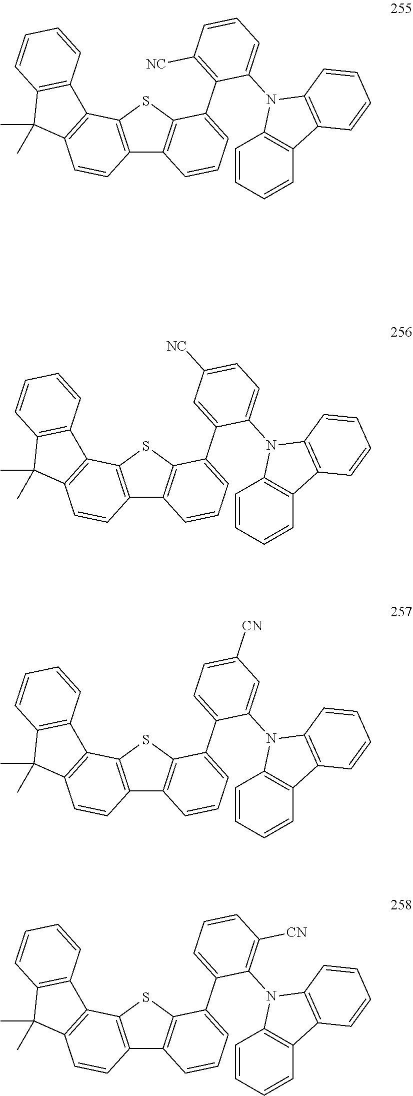 [SMILES: CC1(C)c2ccccc2-c2c1ccc1c2sc2c(-c3c(C#N)cccc3-n3c4ccccc4c4ccccc43)cccc21 ; CC1(C)c2ccccc2-c2c1ccc1c2sc2c(-c3cc(C#N)ccc3-n3c4ccccc4c4ccccc43)cccc21 ; CC1(C)c2ccccc2-c2c1ccc1c2sc2c(-c3ccc(C#N)cc3-n3c4ccccc4c4ccccc43)cccc21 ; CC1(C)c2ccccc2-c2c1ccc1c2sc2c(-c3cccc(C#N)c3-n3c4ccccc4c4ccccc43)cccc21]

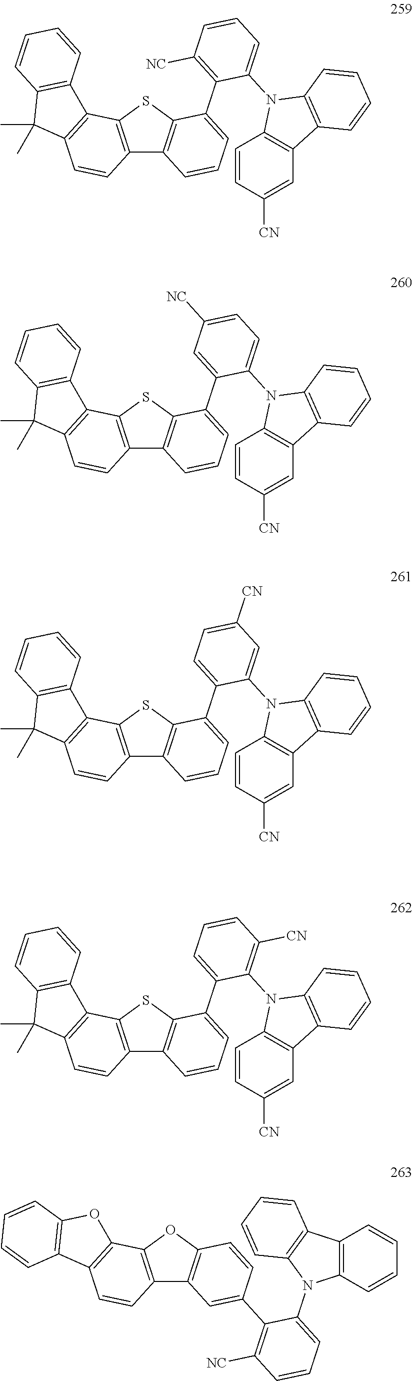

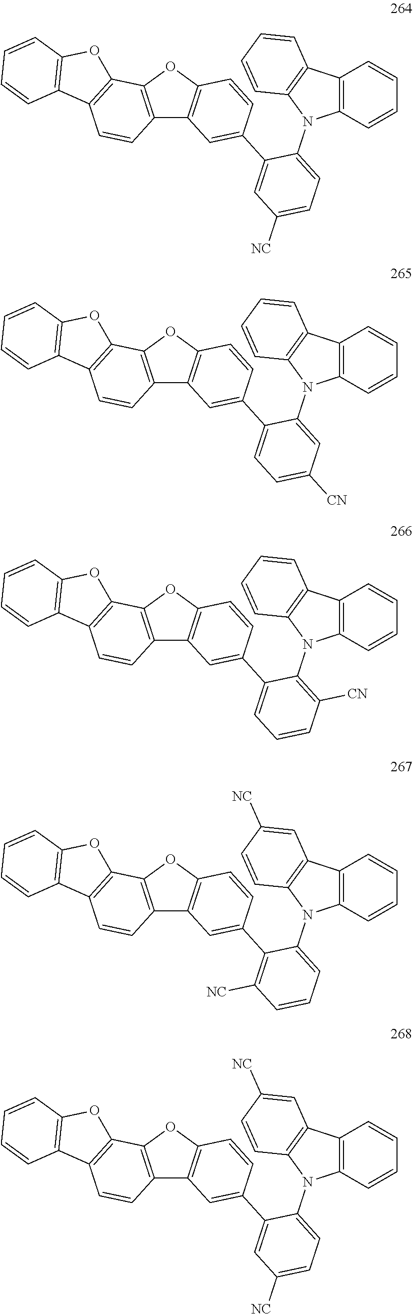

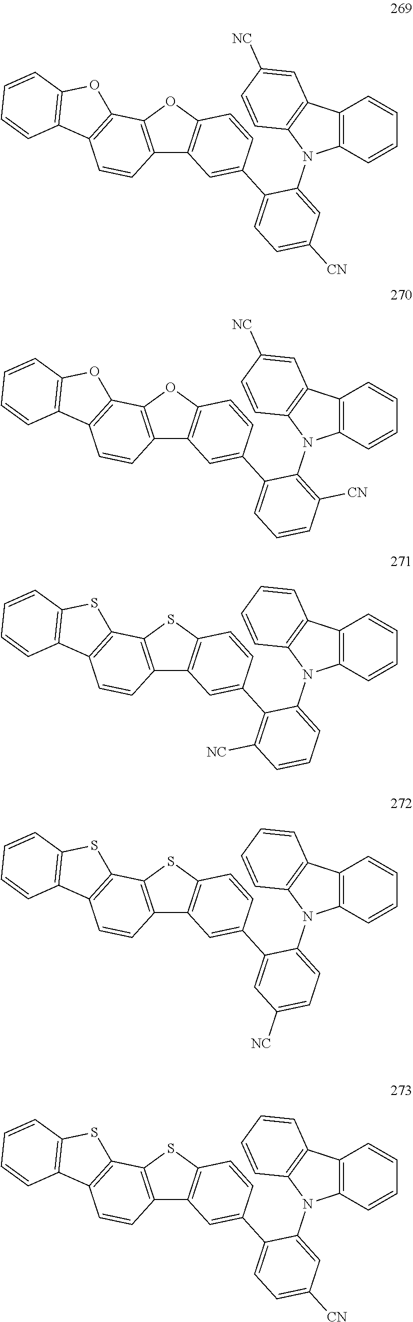

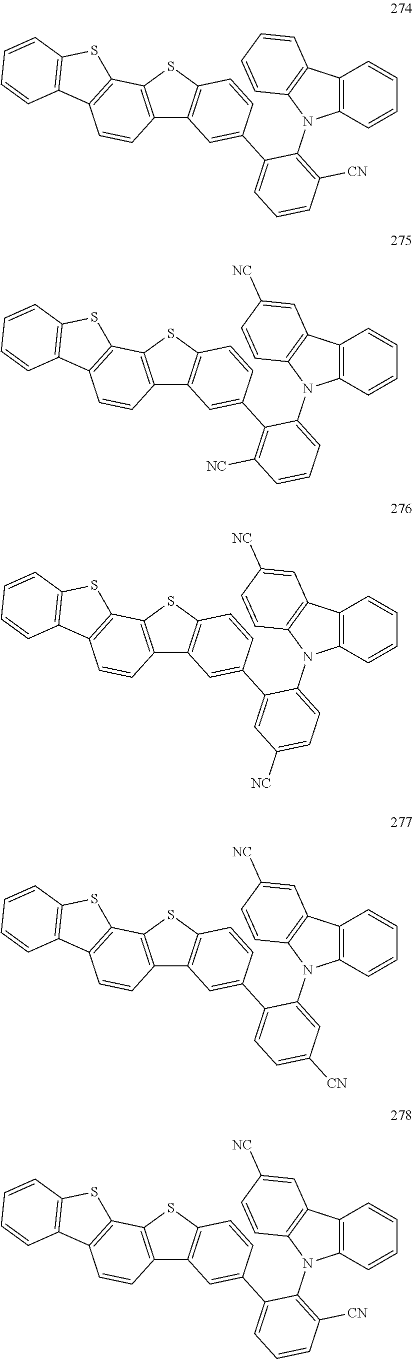

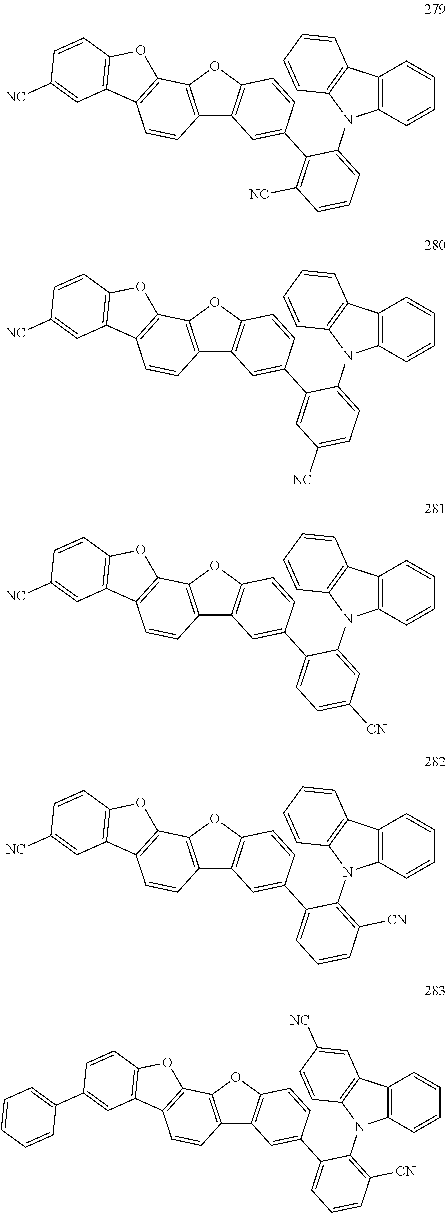

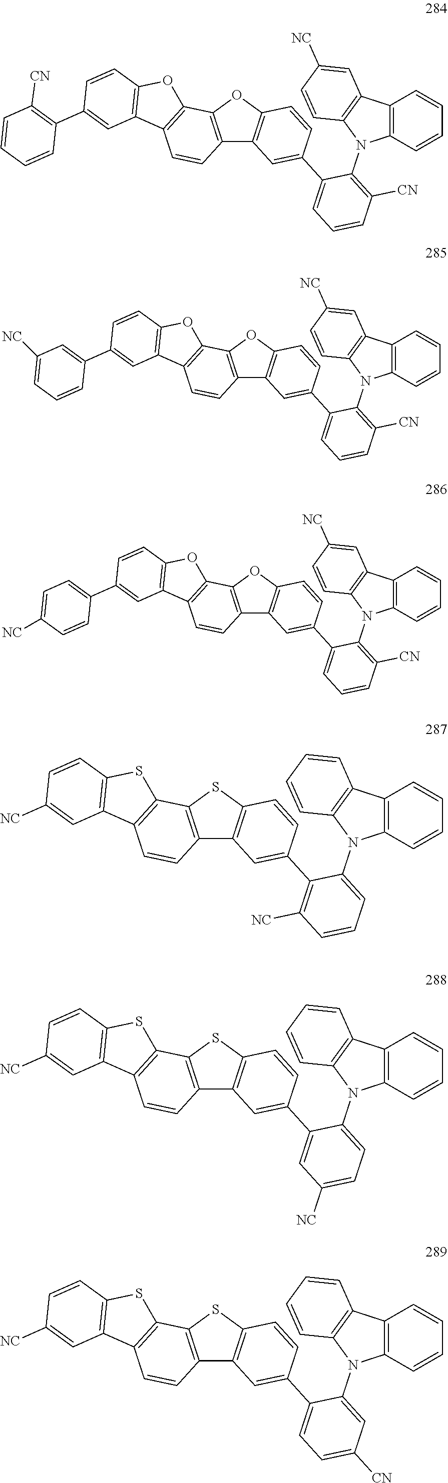

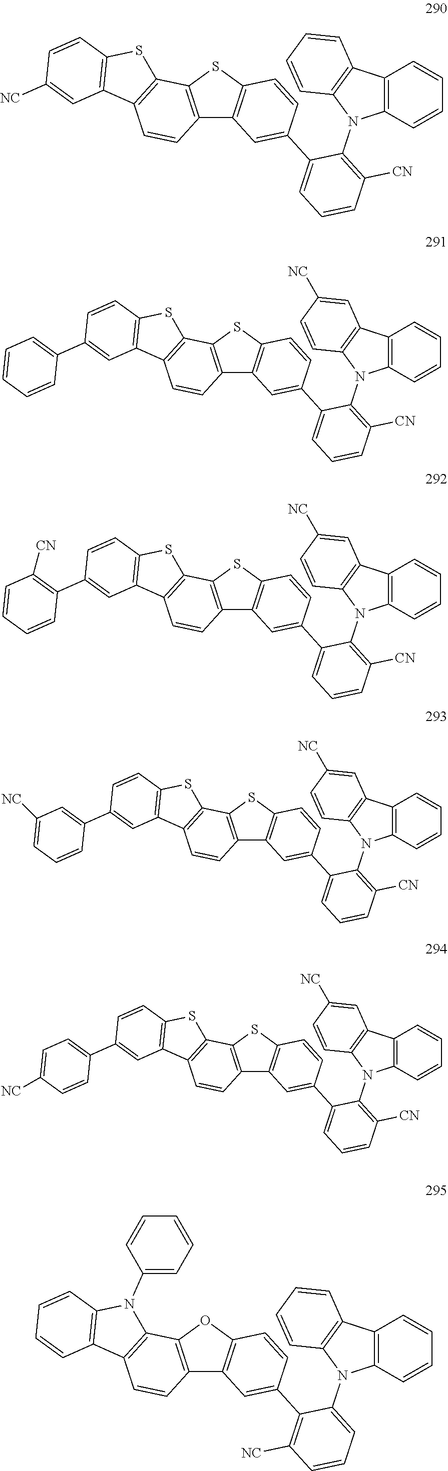

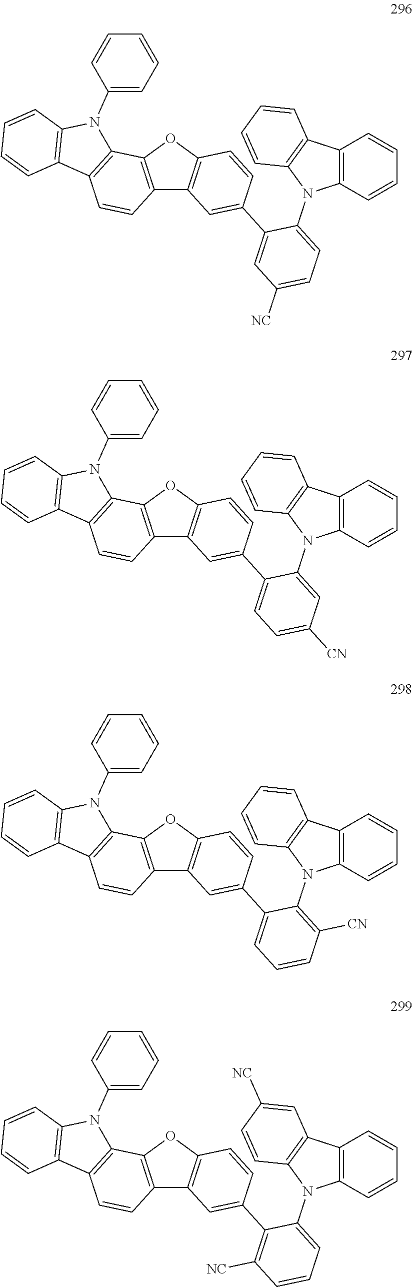

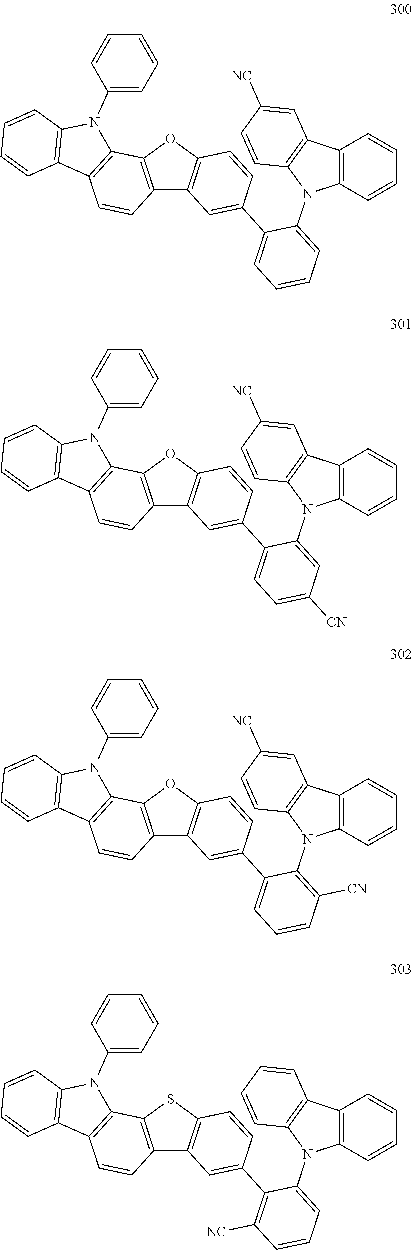

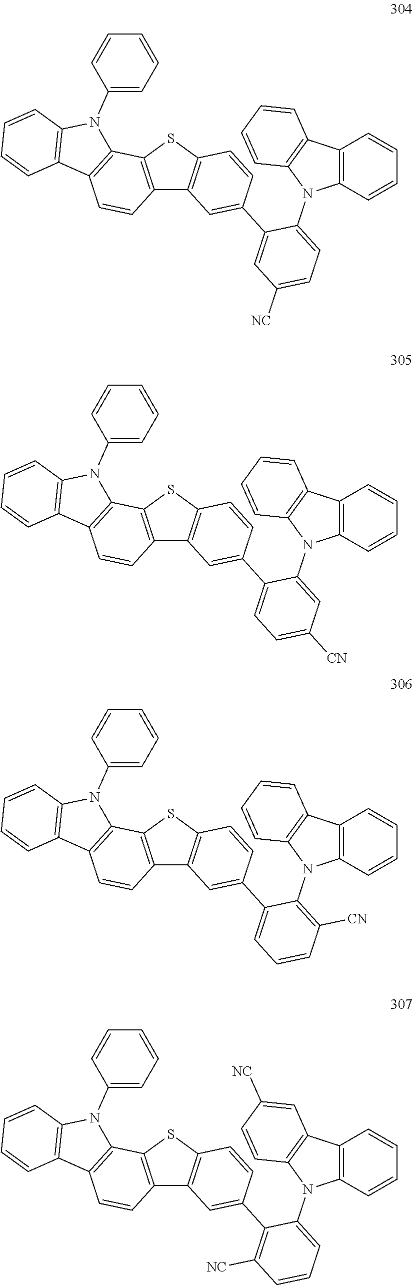

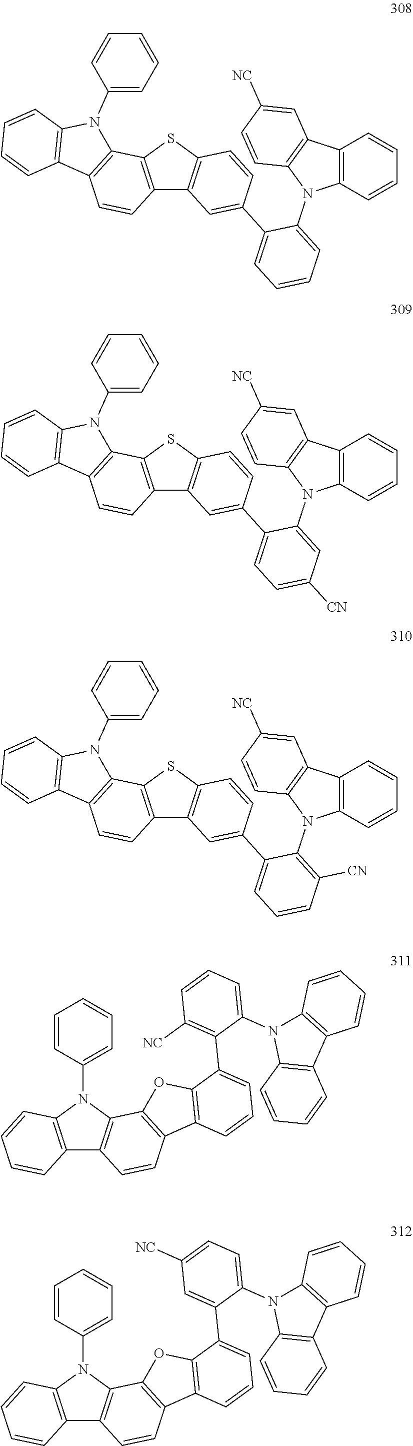

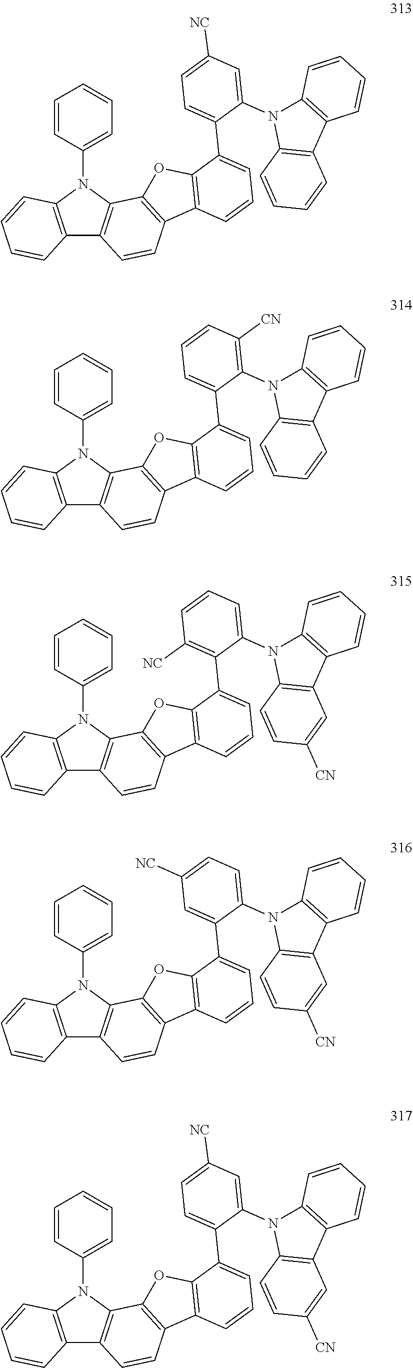 [SMILES: N#Cc1ccc(-c2cccc3c2oc2c3ccc3c4ccccc4n(-c4ccccc4)c32)c(-n2c3ccccc3c3ccccc32)c1 ; N#Cc1cccc(-c2cccc3c2oc2c3ccc3c4ccccc4n(-c4ccccc4)c32)c1-n1c2ccccc2c2ccccc21 ; N#Cc1ccc2c(c1)c1ccccc1n2-c1cccc(C#N)c1-c1cccc2c1oc1c2ccc2c3ccccc3n(-c3ccccc3)c21 ; N#Cc1ccc(-n2c3ccccc3c3cc(C#N)ccc32)c(-c2cccc3c2oc2c3ccc3c4ccccc4n(-c4ccccc4)c32)c1 ; N#Cc1ccc(-c2cccc3c2oc2c3ccc3c4ccccc4n(-c4ccccc4)c32)c(-n2c3ccccc3c3cc(C#N)ccc32)c1]

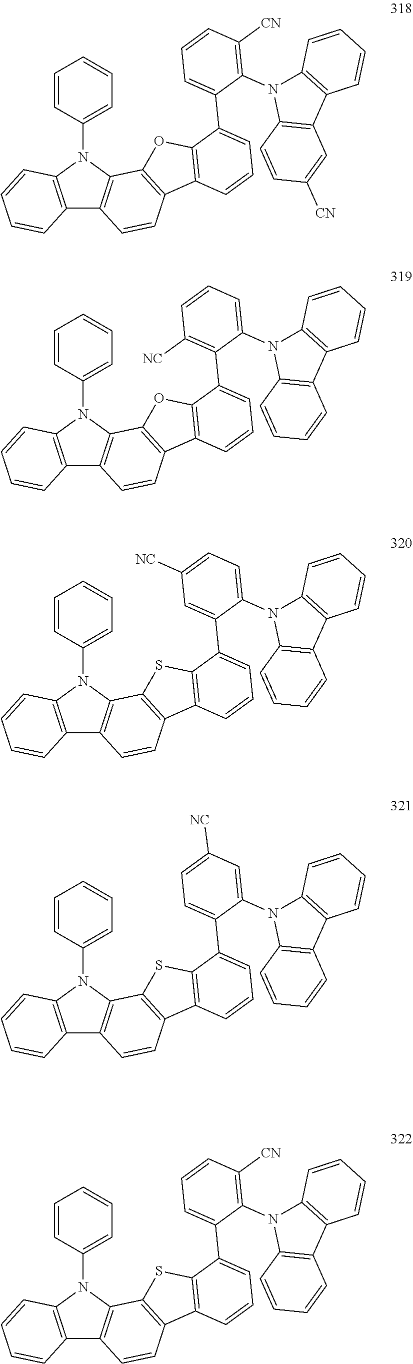 [SMILES: N#Cc1ccc2c(c1)c1ccccc1n2-c1c(C#N)cccc1-c1cccc2c1oc1c2ccc2c3ccccc3n(-c3ccccc3)c21 ; N#Cc1cccc(-n2c3ccccc3c3ccccc32)c1-c1cccc2c1oc1c2ccc2c3ccccc3n(-c3ccccc3)c21 ; N#Cc1ccc(-n2c3ccccc3c3ccccc32)c(-c2cccc3c2sc2c3ccc3c4ccccc4n(-c4ccccc4)c32)c1 ; N#Cc1ccc(-c2cccc3c2sc2c3ccc3c4ccccc4n(-c4ccccc4)c32)c(-n2c3ccccc3c3ccccc32)c1 ; N#Cc1cccc(-c2cccc3c2sc2c3ccc3c4ccccc4n(-c4ccccc4)c32)c1-n1c2ccccc2c2ccccc21]

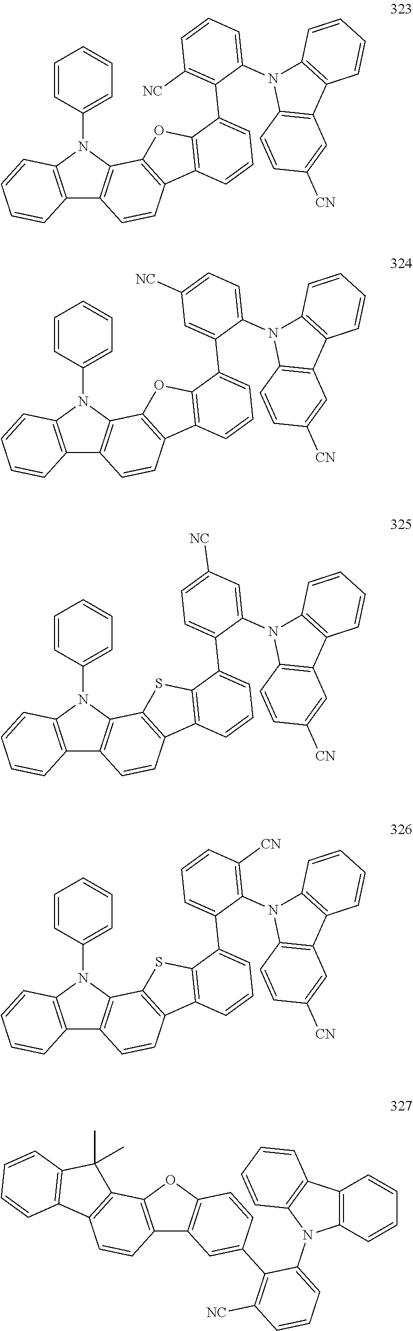

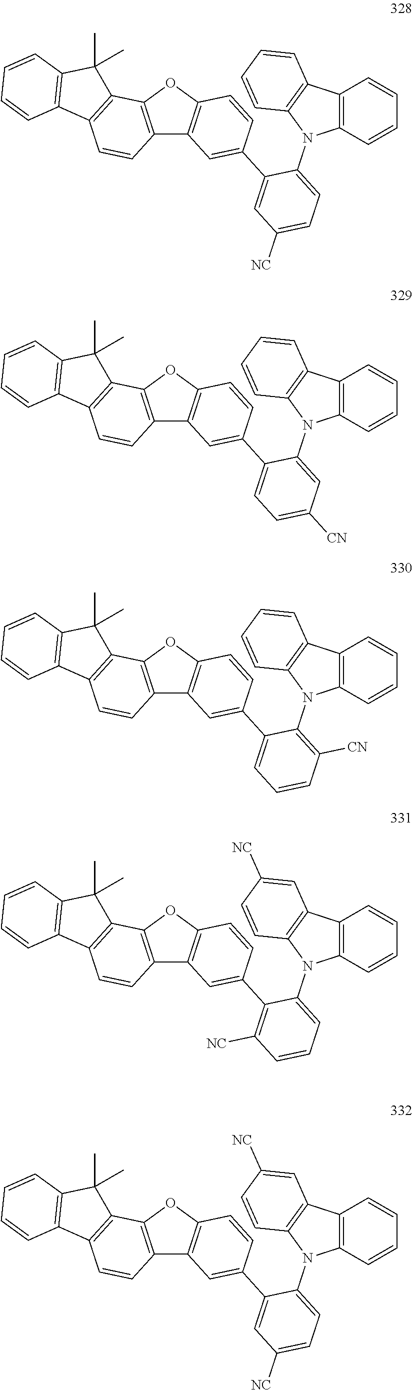

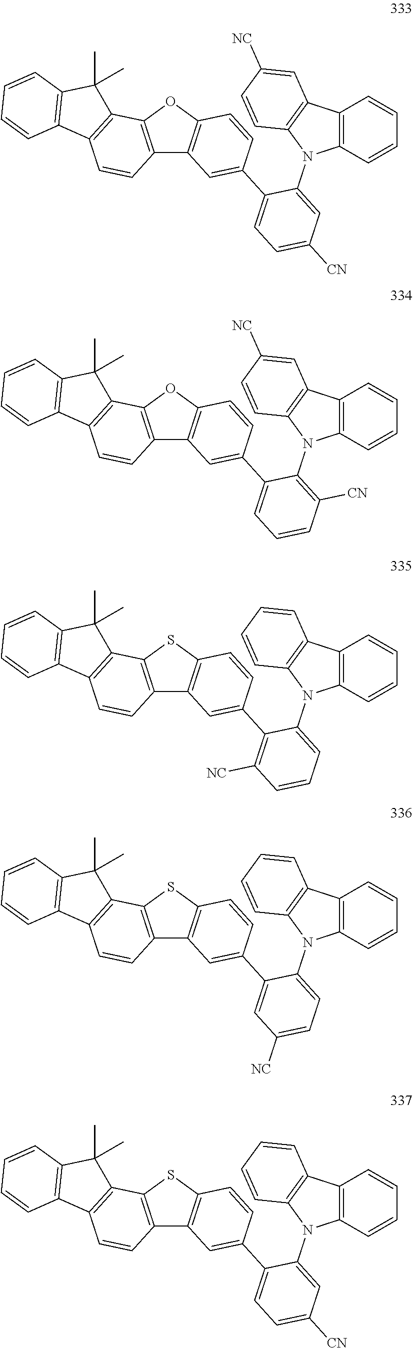

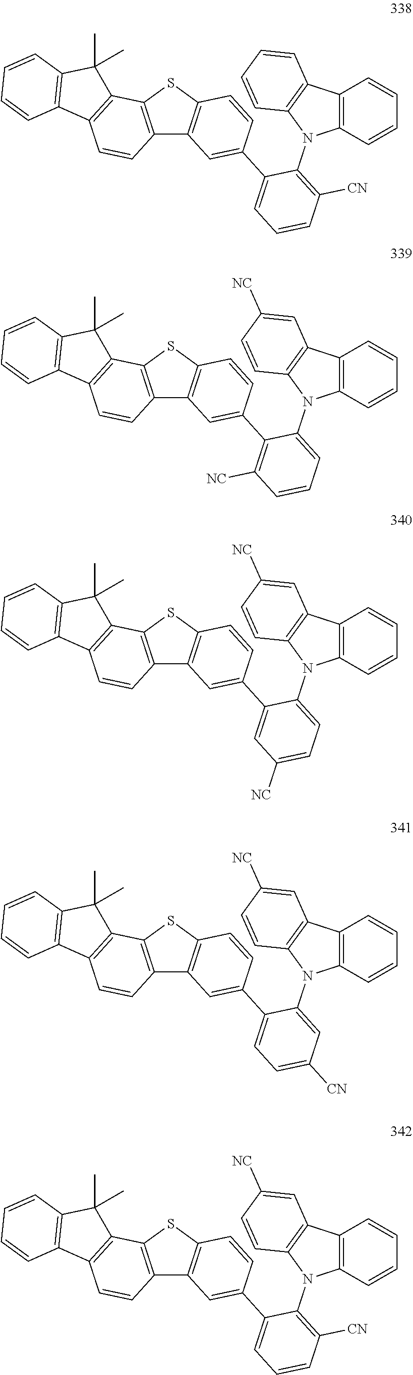

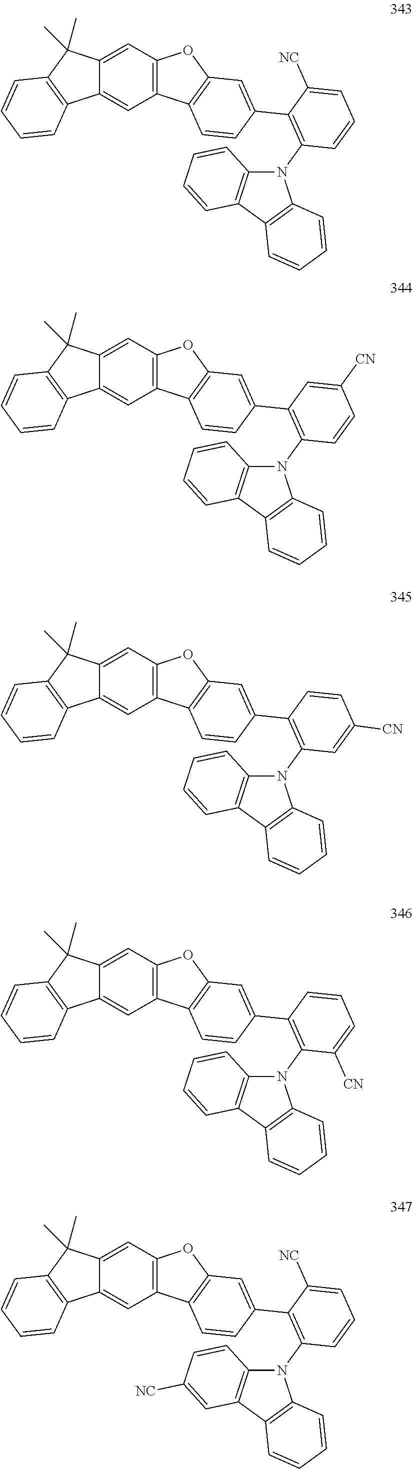 [SMILES: CC1(C)c2ccccc2-c2cc3c(cc21)oc1cc(-c2c(C#N)cccc2-n2c4ccccc4c4ccccc42)ccc13 ; CC1(C)c2ccccc2-c2cc3c(cc21)oc1cc(-c2cc(C#N)ccc2-n2c4ccccc4c4ccccc42)ccc13 ; CC1(C)c2ccccc2-c2cc3c(cc21)oc1cc(-c2ccc(C#N)cc2-n2c4ccccc4c4ccccc42)ccc13 ; CC1(C)c2ccccc2-c2cc3c(cc21)oc1cc(-c2cccc(C#N)c2-n2c4ccccc4c4ccccc42)ccc13 ; CC1(C)c2ccccc2-c2cc3c(cc21)oc1cc(-c2c(C#N)cccc2-n2c4ccccc4c4cc(C#N)ccc42)ccc13]

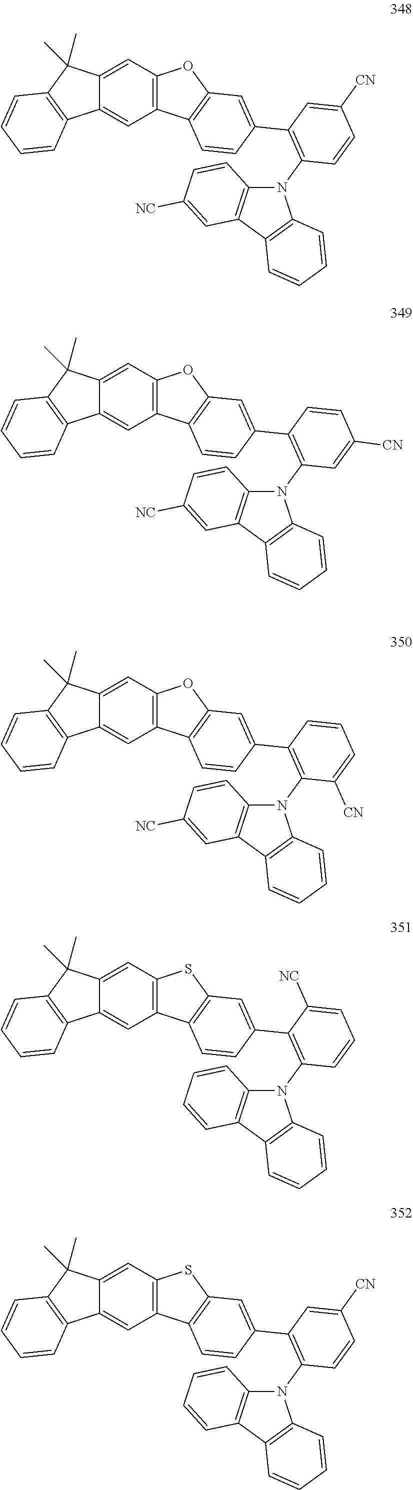 [SMILES: CC1(C)c2ccccc2-c2cc3c(cc21)oc1cc(-c2cc(C#N)ccc2-n2c4ccccc4c4cc(C#N)ccc42)ccc13 ; CC1(C)c2ccccc2-c2cc3c(cc21)oc1cc(-c2ccc(C#N)cc2-n2c4ccccc4c4cc(C#N)ccc42)ccc13 ; CC1(C)c2ccccc2-c2cc3c(cc21)oc1cc(-c2cccc(C#N)c2-n2c4ccccc4c4cc(C#N)ccc42)ccc13 ; CC1(C)c2ccccc2-c2cc3c(cc21)sc1cc(-c2c(C#N)cccc2-n2c4ccccc4c4ccccc42)ccc13 ; CC1(C)c2ccccc2-c2cc3c(cc21)sc1cc(-c2cc(C#N)ccc2-n2c4ccccc4c4ccccc42)ccc13]

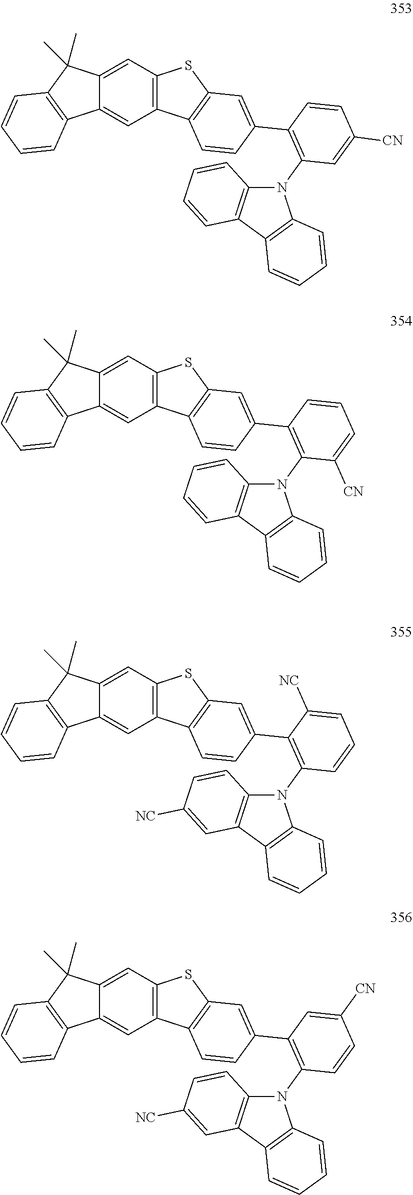 [SMILES: CC1(C)c2ccccc2-c2cc3c(cc21)sc1cc(-c2ccc(C#N)cc2-n2c4ccccc4c4ccccc42)ccc13 ; CC1(C)c2ccccc2-c2cc3c(cc21)sc1cc(-c2cccc(C#N)c2-n2c4ccccc4c4ccccc42)ccc13 ; CC1(C)c2ccccc2-c2cc3c(cc21)sc1cc(-c2c(C#N)cccc2-n2c4ccccc4c4cc(C#N)ccc42)ccc13 ; CC1(C)c2ccccc2-c2cc3c(cc21)sc1cc(-c2cc(C#N)ccc2-n2c4ccccc4c4cc(C#N)ccc42)ccc13]

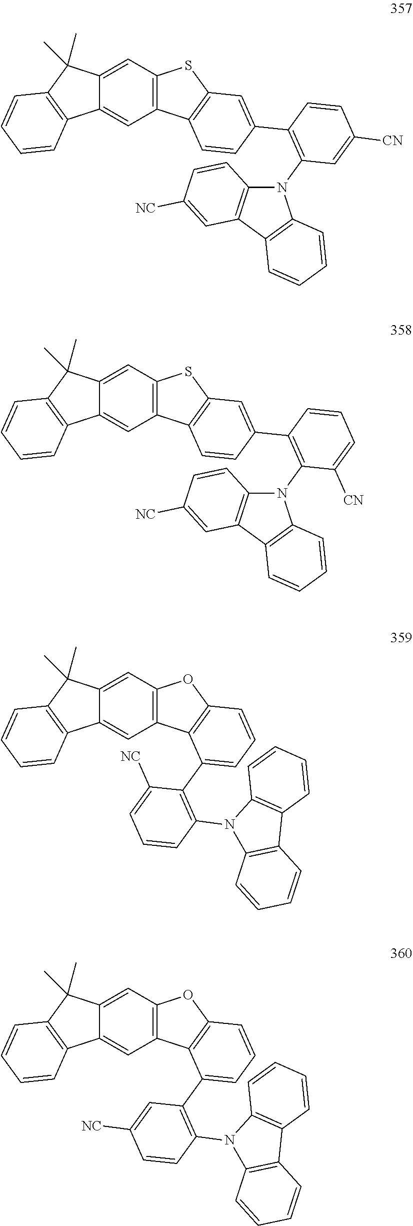 [SMILES: CC1(C)c2ccccc2-c2cc3c(cc21)sc1cc(-c2ccc(C#N)cc2-n2c4ccccc4c4cc(C#N)ccc42)ccc13 ; CC1(C)c2ccccc2-c2cc3c(cc21)sc1cc(-c2cccc(C#N)c2-n2c4ccccc4c4cc(C#N)ccc42)ccc13 ; CC1(C)c2ccccc2-c2cc3c(cc21)oc1cccc(-c2c(C#N)cccc2-n2c4ccccc4c4ccccc42)c13 ; CC1(C)c2ccccc2-c2cc3c(cc21)oc1cccc(-c2cc(C#N)ccc2-n2c4ccccc4c4ccccc42)c13]

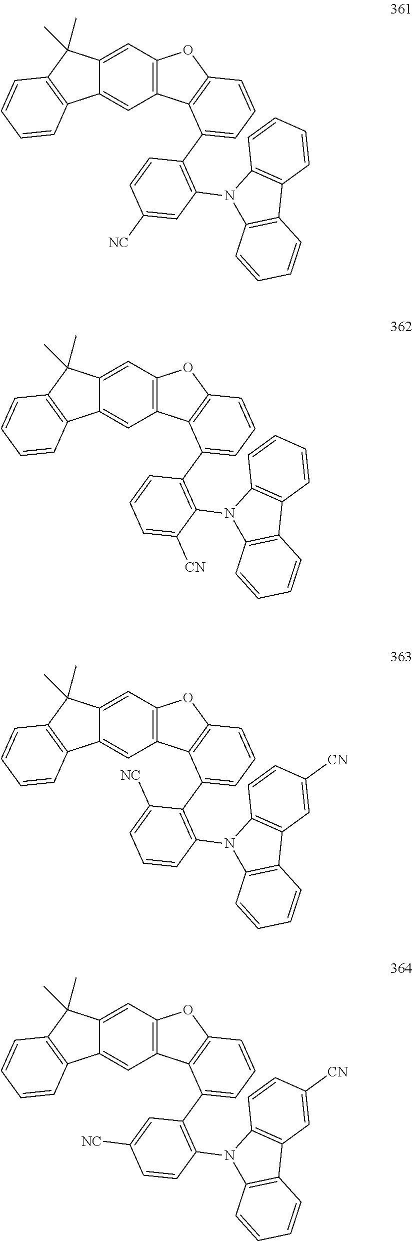 [SMILES: CC1(C)c2ccccc2-c2cc3c(cc21)oc1cccc(-c2ccc(C#N)cc2-n2c4ccccc4c4ccccc42)c13 ; CC1(C)c2ccccc2-c2cc3c(cc21)oc1cccc(-c2cccc(C#N)c2-n2c4ccccc4c4ccccc42)c13 ; CC1(C)c2ccccc2-c2cc3c(cc21)oc1cccc(-c2c(C#N)cccc2-n2c4ccccc4c4cc(C#N)ccc42)c13 ; CC1(C)c2ccccc2-c2cc3c(cc21)oc1cccc(-c2cc(C#N)ccc2-n2c4ccccc4c4cc(C#N)ccc42)c13]

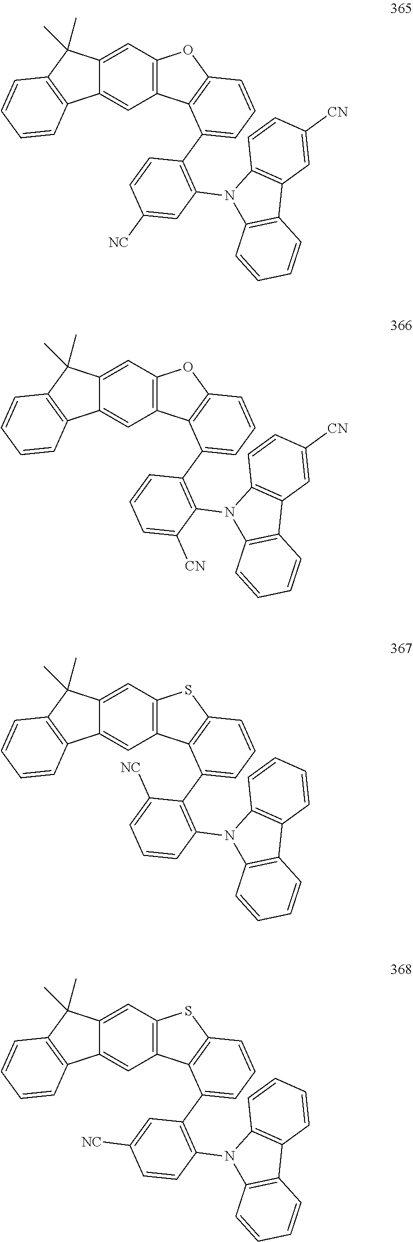 [SMILES: CC1(C)c2ccccc2-c2cc3c(cc21)oc1cccc(-c2ccc(C#N)cc2-n2c4ccccc4c4cc(C#N)ccc42)c13 ; CC1(C)c2ccccc2-c2cc3c(cc21)oc1cccc(-c2cccc(C#N)c2-n2c4ccccc4c4cc(C#N)ccc42)c13 ; CC1(C)c2ccccc2-c2cc3c(cc21)sc1cccc(-c2c(C#N)cccc2-n2c4ccccc4c4ccccc42)c13 ; CC1(C)c2ccccc2-c2cc3c(cc21)sc1cccc(-c2cc(C#N)ccc2-n2c4ccccc4c4ccccc42)c13]

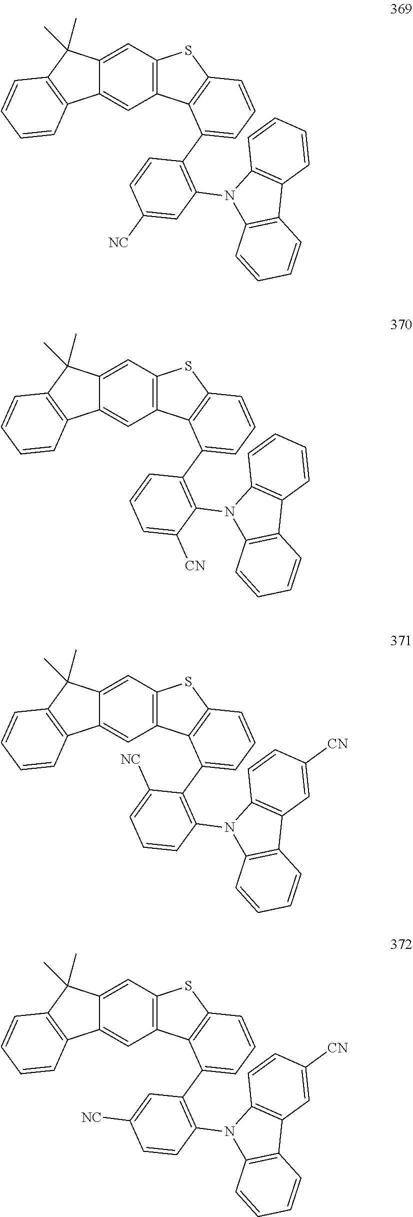 [SMILES: CC1(C)c2ccccc2-c2cc3c(cc21)sc1cccc(-c2ccc(C#N)cc2-n2c4ccccc4c4ccccc42)c13 ; CC1(C)c2ccccc2-c2cc3c(cc21)sc1cccc(-c2cccc(C#N)c2-n2c4ccccc4c4ccccc42)c13 ; CC1(C)c2ccccc2-c2cc3c(cc21)sc1cccc(-c2c(C#N)cccc2-n2c4ccccc4c4cc(C#N)ccc42)c13 ; CC1(C)c2ccccc2-c2cc3c(cc21)sc1cccc(-c2cc(C#N)ccc2-n2c4ccccc4c4cc(C#N)ccc42)c13]

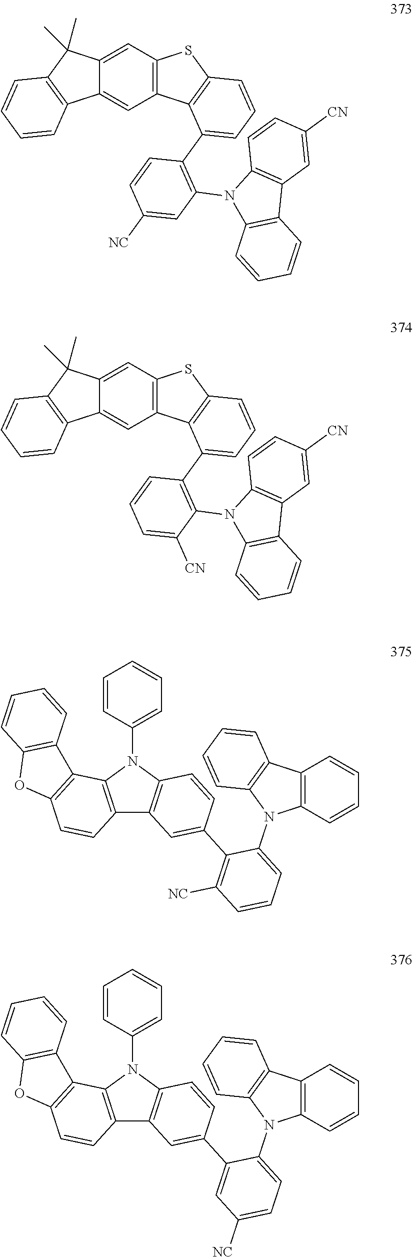 [SMILES: CC1(C)c2ccccc2-c2cc3c(cc21)sc1cccc(-c2ccc(C#N)cc2-n2c4ccccc4c4cc(C#N)ccc42)c13 ; CC1(C)c2ccccc2-c2cc3c(cc21)sc1cccc(-c2cccc(C#N)c2-n2c4ccccc4c4cc(C#N)ccc42)c13 ; N#Cc1cccc(-n2c3ccccc3c3ccccc32)c1-c1ccc2c(c1)c1ccc3oc4ccccc4c3c1n2-c1ccccc1 ; N#Cc1ccc(-n2c3ccccc3c3ccccc32)c(-c2ccc3c(c2)c2ccc4oc5ccccc5c4c2n3-c2ccccc2)c1]

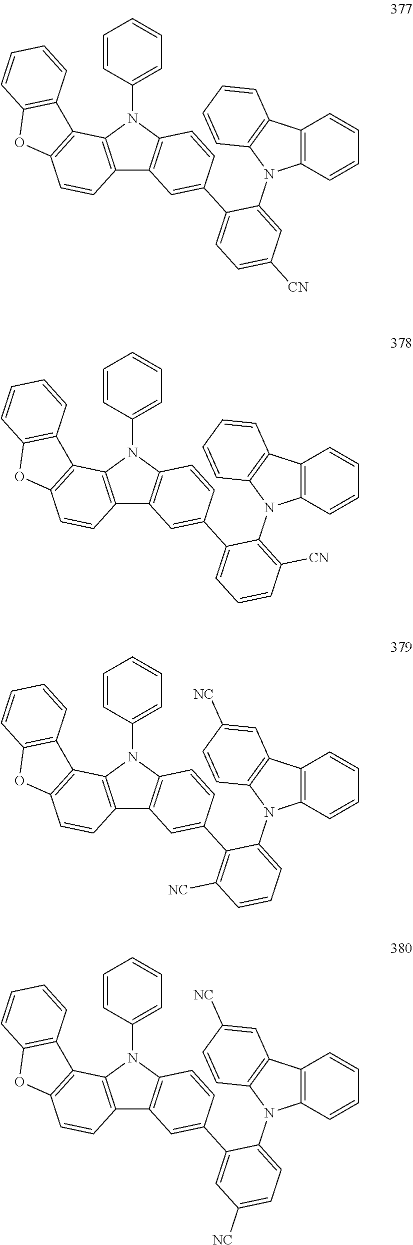 [SMILES: N#Cc1ccc(-c2ccc3c(c2)c2ccc4oc5ccccc5c4c2n3-c2ccccc2)c(-n2c3ccccc3c3ccccc32)c1 ; N#Cc1cccc(-c2ccc3c(c2)c2ccc4oc5ccccc5c4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3oc4ccccc4c3c1n2-c1ccccc1 ; N#Cc1ccc(-n2c3ccccc3c3cc(C#N)ccc32)c(-c2ccc3c(c2)c2ccc4oc5ccccc5c4c2n3-c2ccccc2)c1]

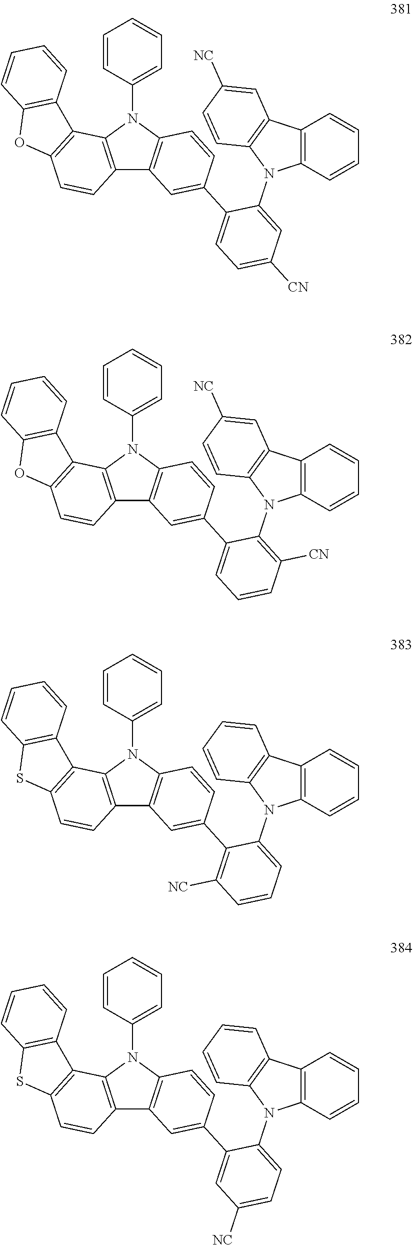 [SMILES: N#Cc1ccc(-c2ccc3c(c2)c2ccc4oc5ccccc5c4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3oc4ccccc4c3c1n2-c1ccccc1 ; N#Cc1cccc(-n2c3ccccc3c3ccccc32)c1-c1ccc2c(c1)c1ccc3sc4ccccc4c3c1n2-c1ccccc1 ; N#Cc1ccc(-n2c3ccccc3c3ccccc32)c(-c2ccc3c(c2)c2ccc4sc5ccccc5c4c2n3-c2ccccc2)c1]

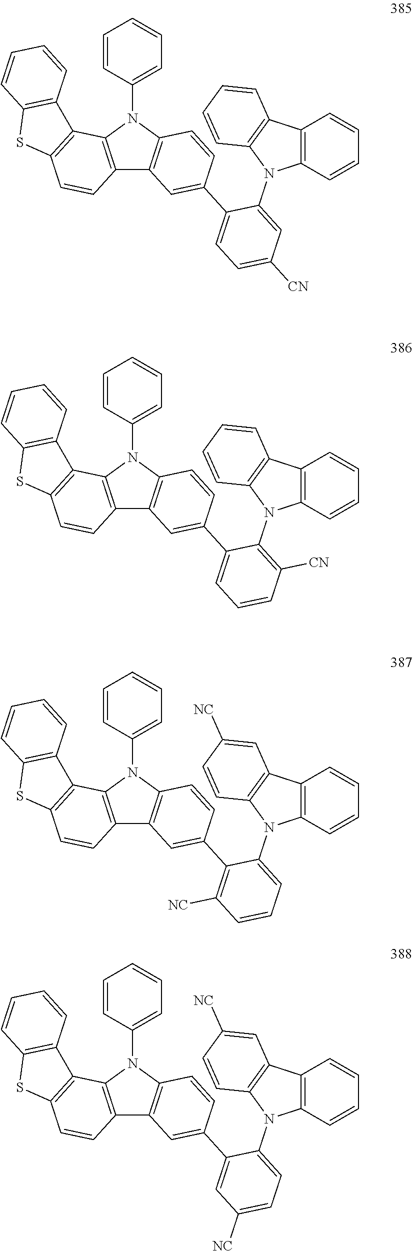 [SMILES: N#Cc1ccc(-c2ccc3c(c2)c2ccc4sc5ccccc5c4c2n3-c2ccccc2)c(-n2c3ccccc3c3ccccc32)c1 ; N#Cc1cccc(-c2ccc3c(c2)c2ccc4sc5ccccc5c4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3sc4ccccc4c3c1n2-c1ccccc1 ; N#Cc1ccc(-n2c3ccccc3c3cc(C#N)ccc32)c(-c2ccc3c(c2)c2ccc4sc5ccccc5c4c2n3-c2ccccc2)c1]

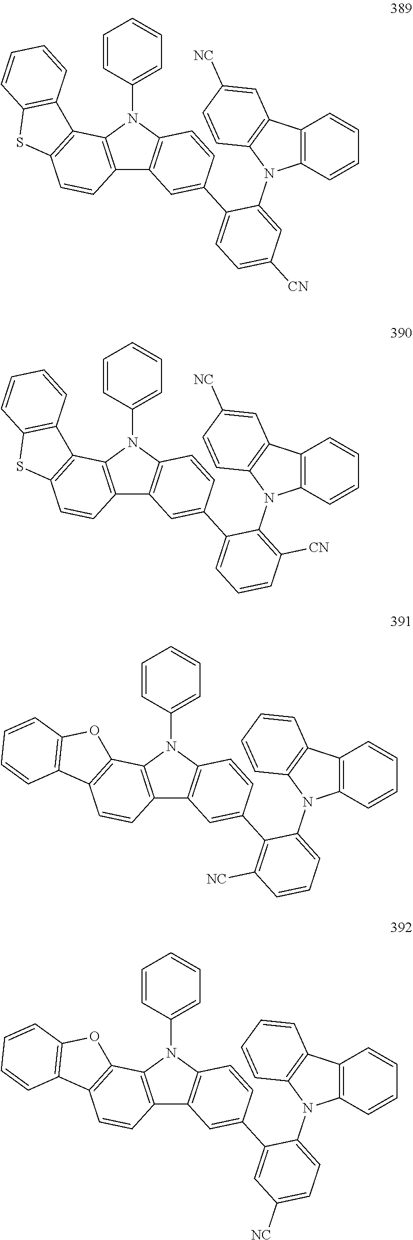 [SMILES: N#Cc1ccc(-c2ccc3c(c2)c2ccc4sc5ccccc5c4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3sc4ccccc4c3c1n2-c1ccccc1 ; N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1]

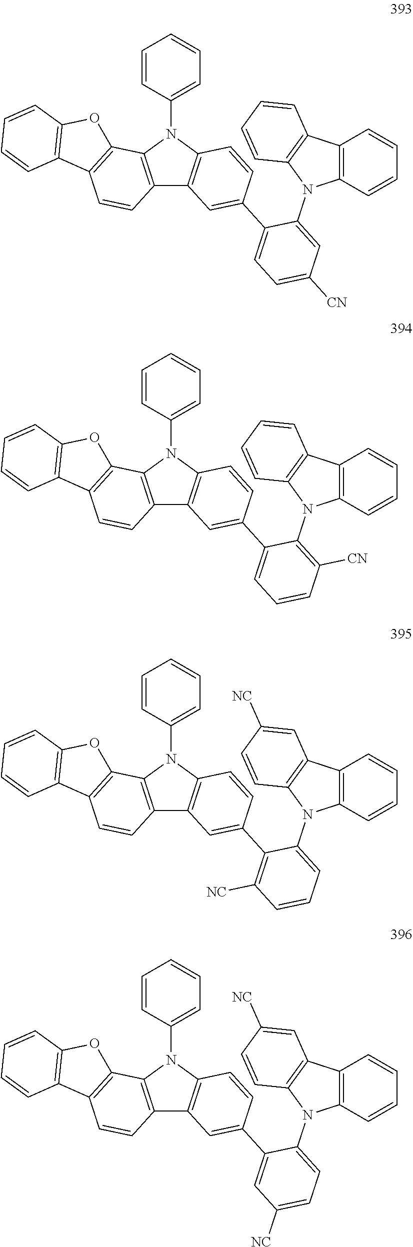 [SMILES: N#Cc1ccc(-c2ccc3c(c2)c2ccc4c5ccccc5oc4c2n3-c2ccccc2)c(-n2c3ccccc3c3ccccc32)c1 ; N#Cc1cccc(-c2ccc3c(c2)c2ccc4c5ccccc5oc4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1]

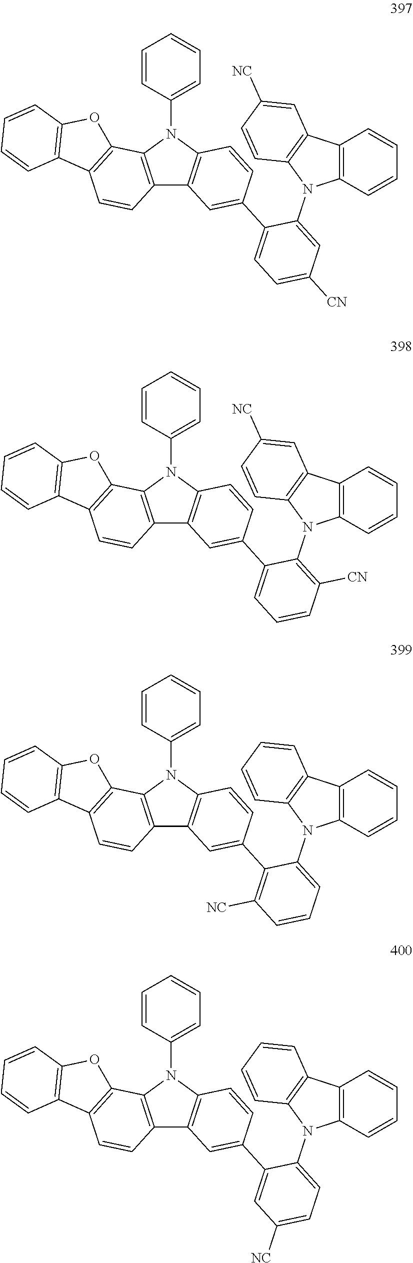 [SMILES: N#Cc1ccc(-c2ccc3c(c2)c2ccc4c5ccccc5oc4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3c4ccccc4oc3c1n2-c1ccccc1 ; N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1]

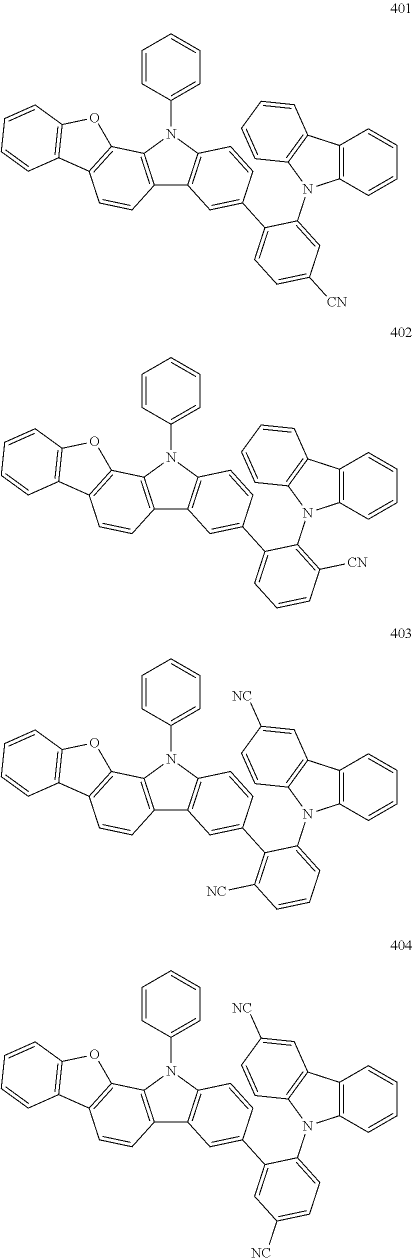 [SMILES: N#Cc1ccc(-c2ccc3c(c2)c2ccc4c5ccccc5oc4c2n3-c2ccccc2)c(-n2c3ccccc3c3ccccc32)c1 ; N#Cc1cccc(-c2ccc3c(c2)c2ccc4c5ccccc5oc4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1]

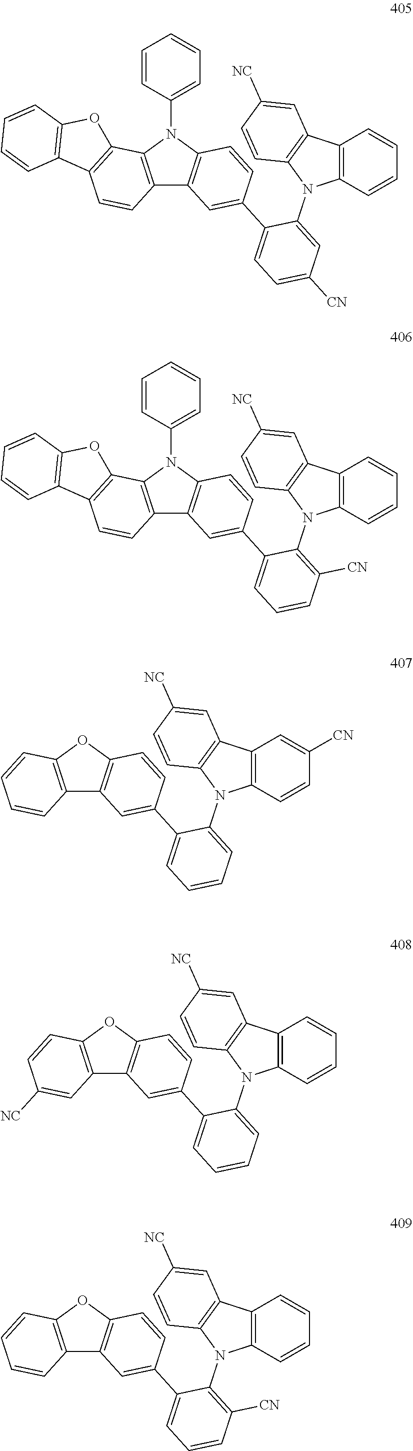

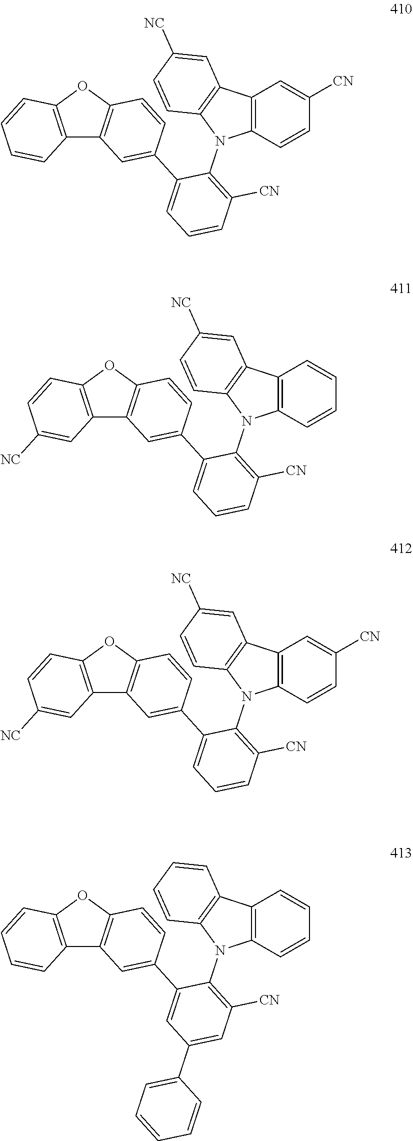

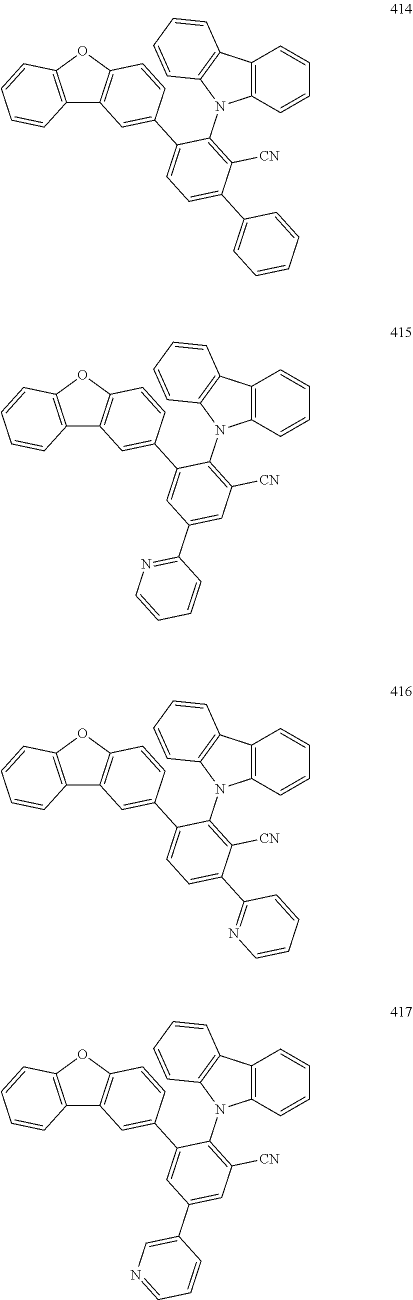

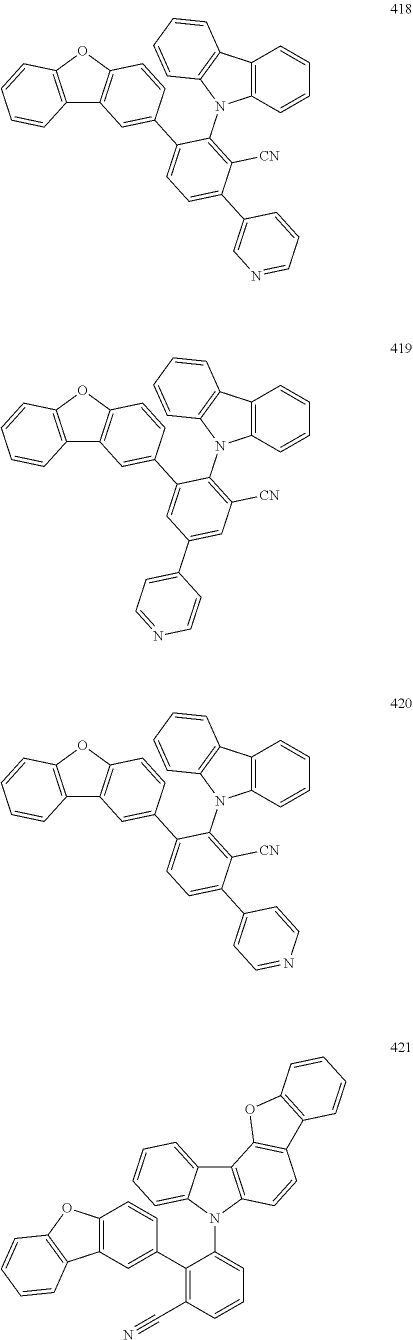

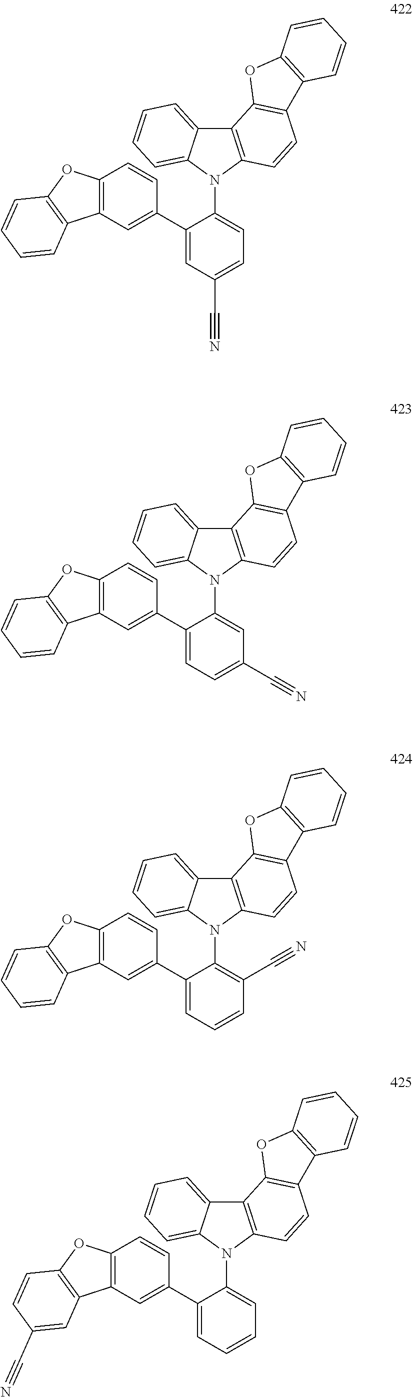 [SMILES: N#Cc1ccc(-n2c3ccccc3c3c4oc5ccccc5c4ccc32)c(-c2ccc3oc4ccccc4c3c2)c1 ; N#Cc1ccc(-c2ccc3oc4ccccc4c3c2)c(-n2c3ccccc3c3c4oc5ccccc5c4ccc32)c1 ; N#Cc1cccc(-c2ccc3oc4ccccc4c3c2)c1-n1c2ccccc2c2c3oc4ccccc4c3ccc21 ; N#Cc1ccc2oc3ccc(-c4ccccc4-n4c5ccccc5c5c6oc7ccccc7c6ccc54)cc3c2c1]

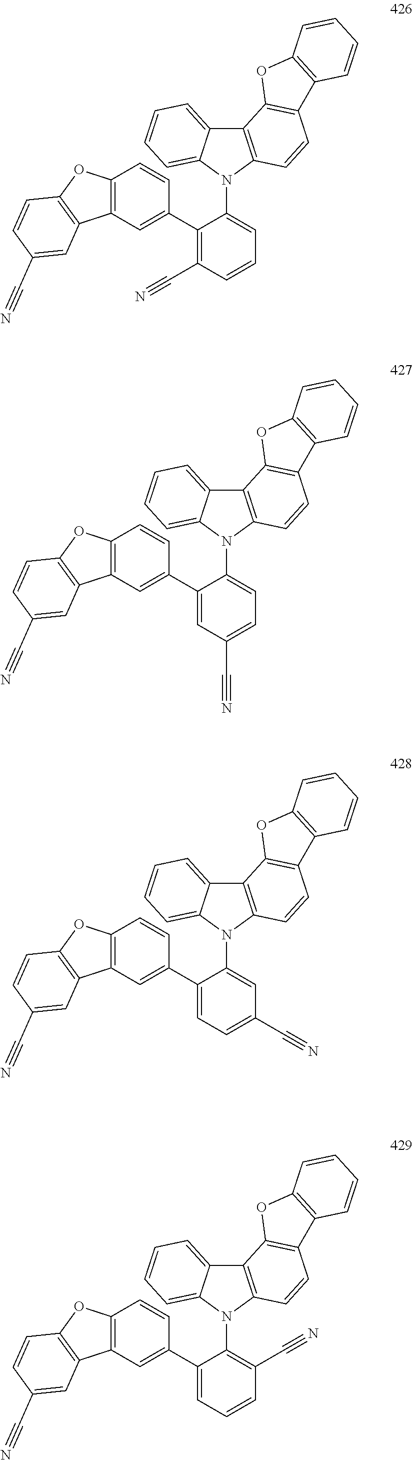 [SMILES: N#Cc1ccc2oc3ccc(-c4c(C#N)cccc4-n4c5ccccc5c5c6oc7ccccc7c6ccc54)cc3c2c1 ; N#Cc1ccc(-n2c3ccccc3c3c4oc5ccccc5c4ccc32)c(-c2ccc3oc4ccc(C#N)cc4c3c2)c1 ; N#Cc1ccc(-c2ccc3oc4ccc(C#N)cc4c3c2)c(-n2c3ccccc3c3c4oc5ccccc5c4ccc32)c1 ; N#Cc1ccc2oc3ccc(-c4cccc(C#N)c4-n4c5ccccc5c5c6oc7ccccc7c6ccc54)cc3c2c1]

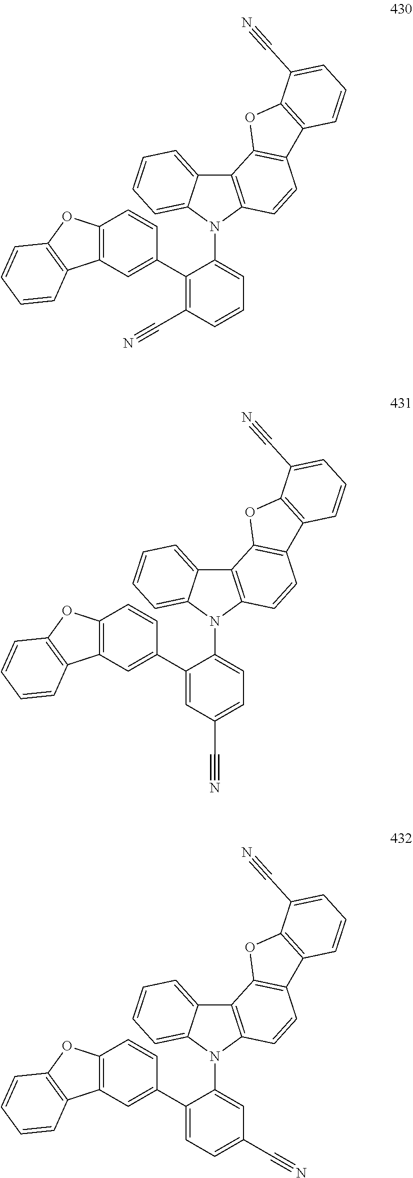 [SMILES: N#Cc1cccc(-n2c3ccccc3c3c4oc5c(C#N)cccc5c4ccc32)c1-c1ccc2oc3ccccc3c2c1 ; N#Cc1ccc(-n2c3ccccc3c3c4oc5c(C#N)cccc5c4ccc32)c(-c2ccc3oc4ccccc4c3c2)c1 ; N#Cc1ccc(-c2ccc3oc4ccccc4c3c2)c(-n2c3ccccc3c3c4oc5c(C#N)cccc5c4ccc32)c1]

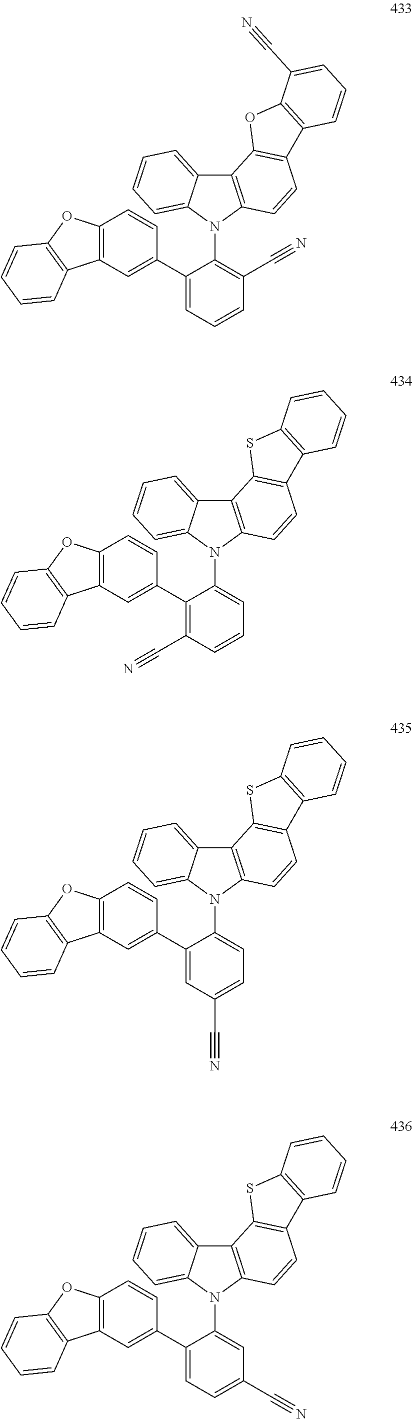 [SMILES: N#Cc1cccc(-c2ccc3oc4ccccc4c3c2)c1-n1c2ccccc2c2c3oc4c(C#N)cccc4c3ccc21 ; N#Cc1cccc(-n2c3ccccc3c3c4sc5ccccc5c4ccc32)c1-c1ccc2oc3ccccc3c2c1 ; N#Cc1ccc(-n2c3ccccc3c3c4sc5ccccc5c4ccc32)c(-c2ccc3oc4ccccc4c3c2)c1 ; N#Cc1ccc(-c2ccc3oc4ccccc4c3c2)c(-n2c3ccccc3c3c4sc5ccccc5c4ccc32)c1]

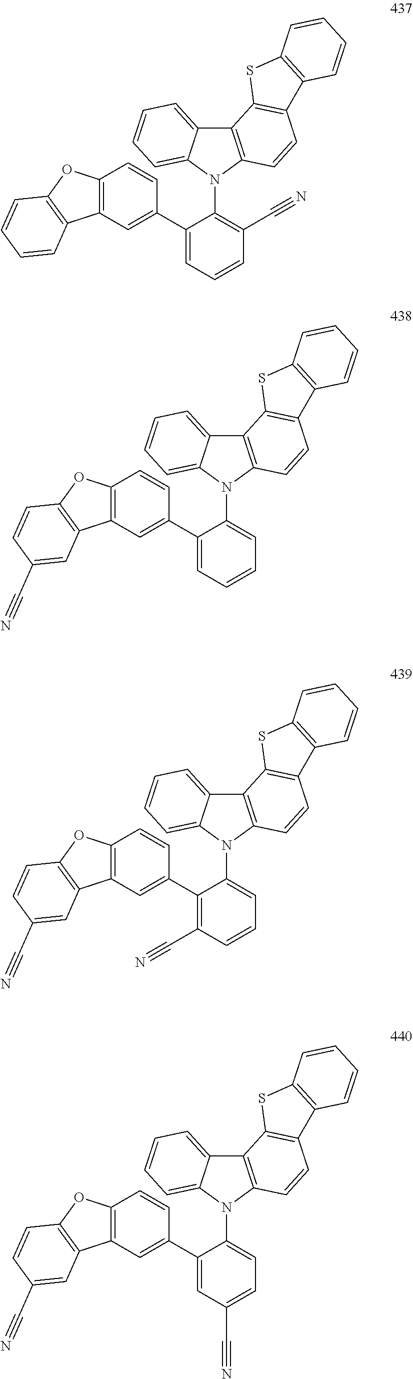 [SMILES: N#Cc1cccc(-c2ccc3oc4ccccc4c3c2)c1-n1c2ccccc2c2c3sc4ccccc4c3ccc21 ; N#Cc1ccc2oc3ccc(-c4ccccc4-n4c5ccccc5c5c6sc7ccccc7c6ccc54)cc3c2c1 ; N#Cc1ccc2oc3ccc(-c4c(C#N)cccc4-n4c5ccccc5c5c6sc7ccccc7c6ccc54)cc3c2c1 ; N#Cc1ccc(-n2c3ccccc3c3c4sc5ccccc5c4ccc32)c(-c2ccc3oc4ccc(C#N)cc4c3c2)c1]

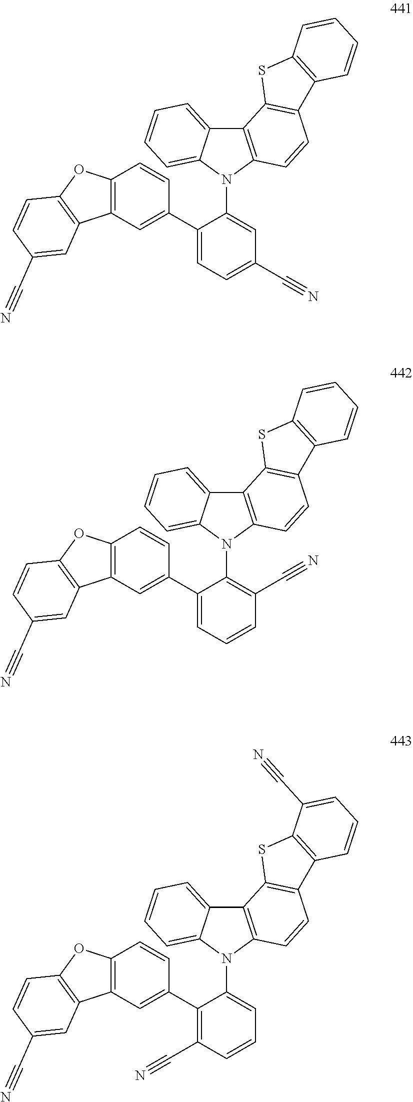 [SMILES: N#Cc1ccc(-c2ccc3oc4ccc(C#N)cc4c3c2)c(-n2c3ccccc3c3c4sc5ccccc5c4ccc32)c1 ; N#Cc1ccc2oc3ccc(-c4cccc(C#N)c4-n4c5ccccc5c5c6sc7ccccc7c6ccc54)cc3c2c1 ; N#Cc1ccc2oc3ccc(-c4c(C#N)cccc4-n4c5ccccc5c5c6sc7c(C#N)cccc7c6ccc54)cc3c2c1]

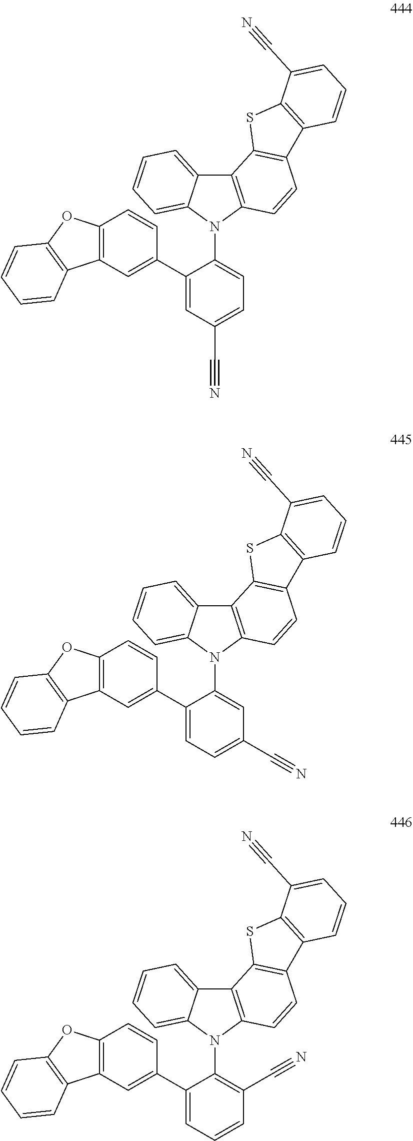 [SMILES: N#Cc1ccc(-n2c3ccccc3c3c4sc5c(C#N)cccc5c4ccc32)c(-c2ccc3oc4ccccc4c3c2)c1 ; N#Cc1ccc(-c2ccc3oc4ccccc4c3c2)c(-n2c3ccccc3c3c4sc5c(C#N)cccc5c4ccc32)c1 ; N#Cc1cccc(-c2ccc3oc4ccccc4c3c2)c1-n1c2ccccc2c2c3sc4c(C#N)cccc4c3ccc21]

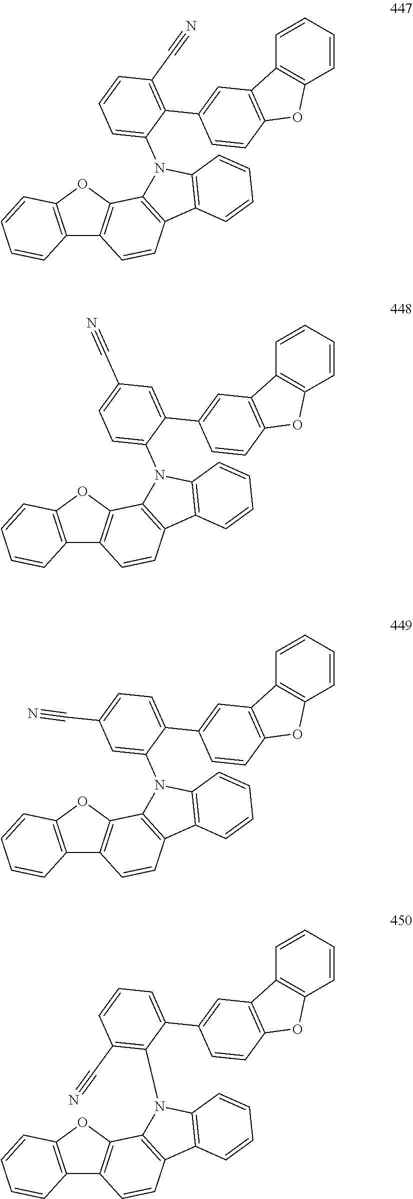 [SMILES: N#Cc1cccc(-n2c3ccccc3c3ccc4c5ccccc5oc4c32)c1-c1ccc2oc3ccccc3c2c1 ; N#Cc1ccc(-n2c3ccccc3c3ccc4c5ccccc5oc4c32)c(-c2ccc3oc4ccccc4c3c2)c1 ; N#Cc1ccc(-c2ccc3oc4ccccc4c3c2)c(-n2c3ccccc3c3ccc4c5ccccc5oc4c32)c1 ; N#Cc1cccc(-c2ccc3oc4ccccc4c3c2)c1-n1c2ccccc2c2ccc3c4ccccc4oc3c21]

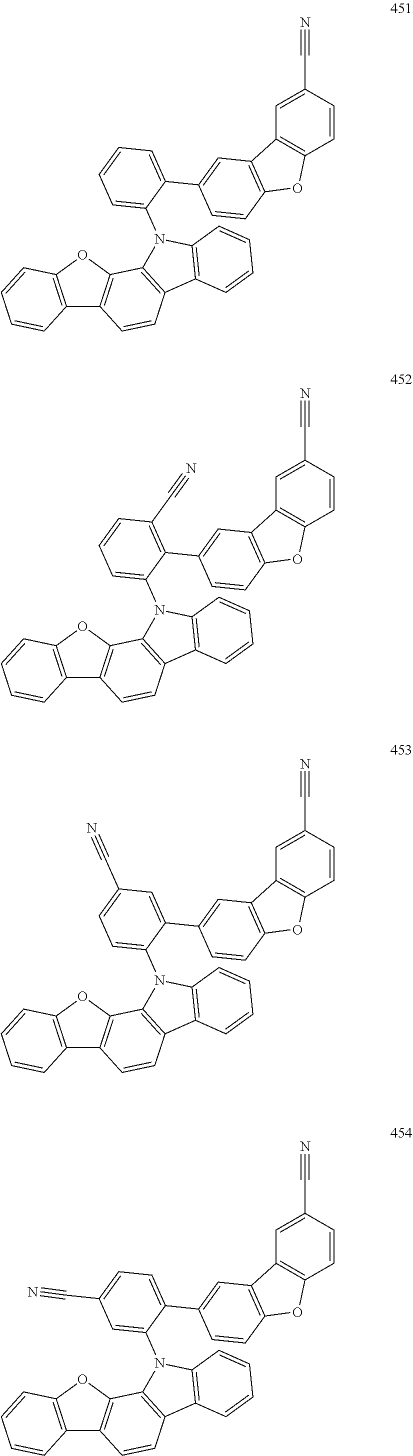 [SMILES: N#Cc1ccc2oc3ccc(-c4ccccc4-n4c5ccccc5c5ccc6c7ccccc7oc6c54)cc3c2c1 ; N#Cc1ccc2oc3ccc(-c4c(C#N)cccc4-n4c5ccccc5c5ccc6c7ccccc7oc6c54)cc3c2c1 ; N#Cc1ccc(-n2c3ccccc3c3ccc4c5ccccc5oc4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4c5ccccc5oc4c32)c1]

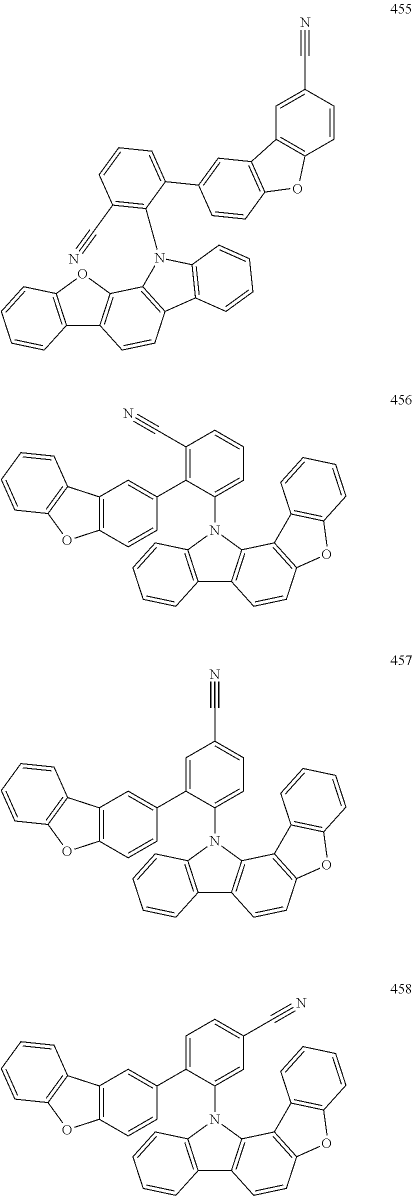 [SMILES: N#Cc1ccc2oc3ccc(-c4cccc(C#N)c4-n4c5ccccc5c5ccc6c7ccccc7oc6c54)cc3c2c1 ; N#Cc1cccc(-n2c3ccccc3c3ccc4oc5ccccc5c4c32)c1-c1ccc2oc3ccccc3c2c1 ; N#Cc1ccc(-n2c3ccccc3c3ccc4oc5ccccc5c4c32)c(-c2ccc3oc4ccccc4c3c2)c1 ; N#Cc1ccc(-c2ccc3oc4ccccc4c3c2)c(-n2c3ccccc3c3ccc4oc5ccccc5c4c32)c1]

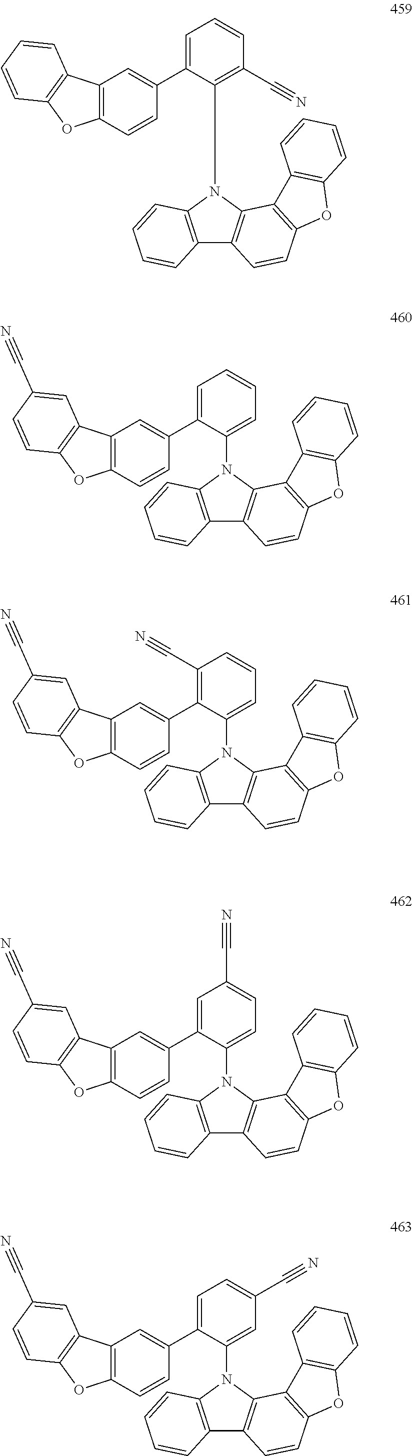 [SMILES: N#Cc1cccc(-c2ccc3oc4ccccc4c3c2)c1-n1c2ccccc2c2ccc3oc4ccccc4c3c21 ; N#Cc1ccc2oc3ccc(-c4ccccc4-n4c5ccccc5c5ccc6oc7ccccc7c6c54)cc3c2c1 ; N#Cc1ccc2oc3ccc(-c4c(C#N)cccc4-n4c5ccccc5c5ccc6oc7ccccc7c6c54)cc3c2c1 ; N#Cc1ccc(-n2c3ccccc3c3ccc4oc5ccccc5c4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4oc5ccccc5c4c32)c1]

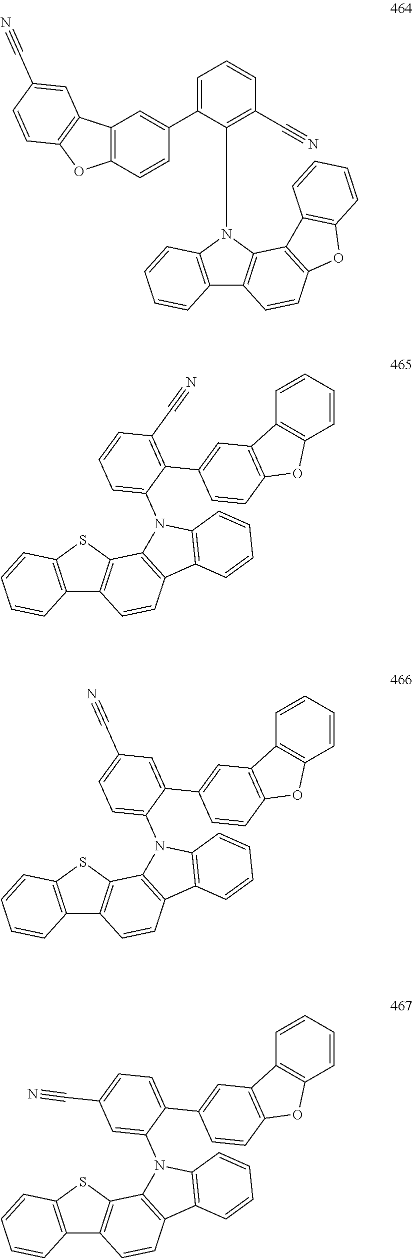 [SMILES: N#Cc1ccc2oc3ccc(-c4cccc(C#N)c4-n4c5ccccc5c5ccc6oc7ccccc7c6c54)cc3c2c1 ; N#Cc1cccc(-n2c3ccccc3c3ccc4c5ccccc5sc4c32)c1-c1ccc2oc3ccccc3c2c1 ; N#Cc1ccc(-n2c3ccccc3c3ccc4c5ccccc5sc4c32)c(-c2ccc3oc4ccccc4c3c2)c1 ; N#Cc1ccc(-c2ccc3oc4ccccc4c3c2)c(-n2c3ccccc3c3ccc4c5ccccc5sc4c32)c1]

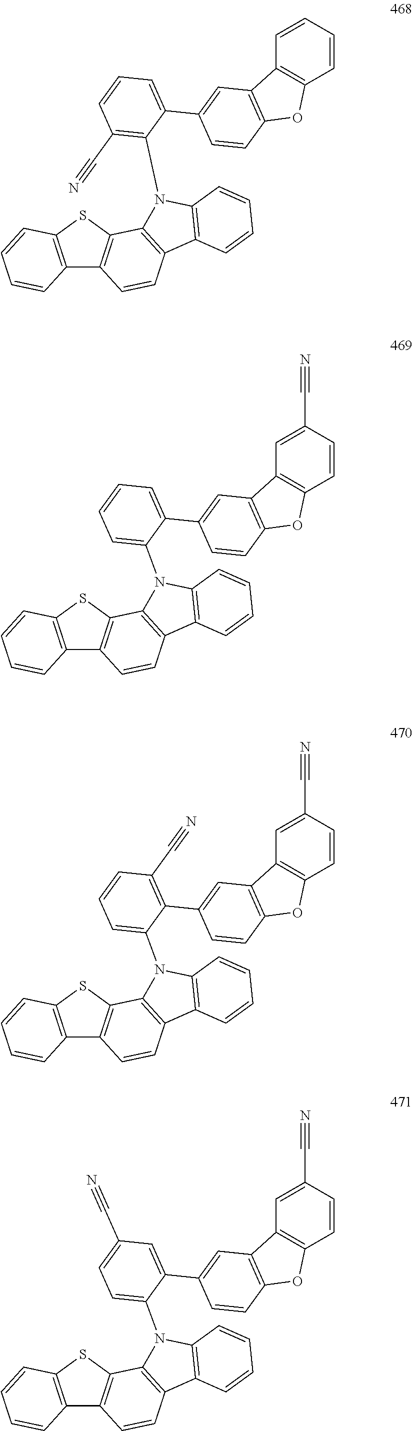 [SMILES: N#Cc1cccc(-c2ccc3oc4ccccc4c3c2)c1-n1c2ccccc2c2ccc3c4ccccc4sc3c21 ; N#Cc1ccc2oc3ccc(-c4ccccc4-n4c5ccccc5c5ccc6c7ccccc7sc6c54)cc3c2c1 ; N#Cc1ccc2oc3ccc(-c4c(C#N)cccc4-n4c5ccccc5c5ccc6c7ccccc7sc6c54)cc3c2c1 ; N#Cc1ccc(-n2c3ccccc3c3ccc4c5ccccc5sc4c32)c(-c2ccc3oc4ccc(C#N)cc4c3c2)c1]

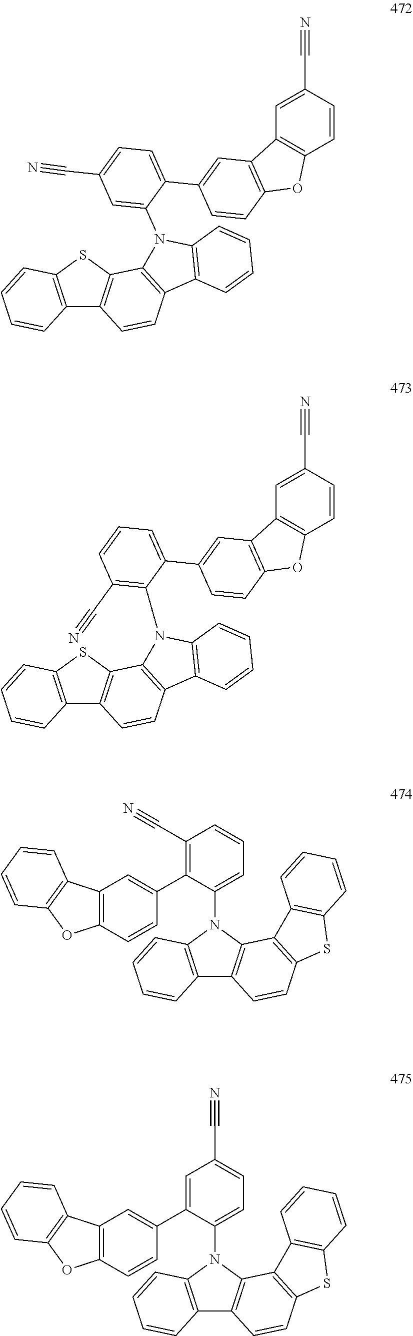 [SMILES: N#Cc1ccc(-c2ccc3oc4ccc(C#N)cc4c3c2)c(-n2c3ccccc3c3ccc4c5ccccc5sc4c32)c1 ; N#Cc1ccc2oc3ccc(-c4cccc(C#N)c4-n4c5ccccc5c5ccc6c7ccccc7sc6c54)cc3c2c1 ; N#Cc1cccc(-n2c3ccccc3c3ccc4sc5ccccc5c4c32)c1-c1ccc2oc3ccccc3c2c1 ; N#Cc1ccc(-n2c3ccccc3c3ccc4sc5ccccc5c4c32)c(-c2ccc3oc4ccccc4c3c2)c1]

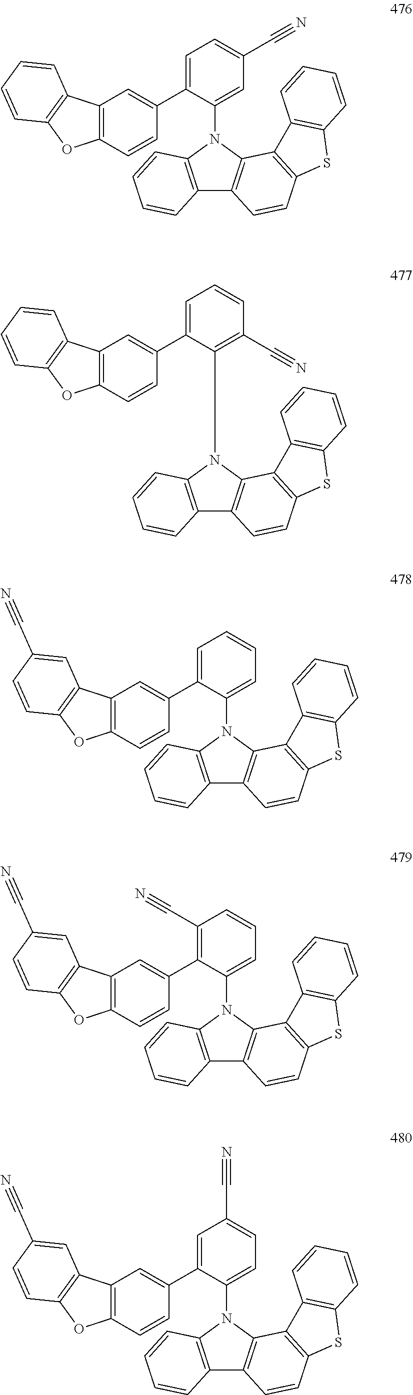 [SMILES: N#Cc1ccc(-c2ccc3oc4ccccc4c3c2)c(-n2c3ccccc3c3ccc4sc5ccccc5c4c32)c1 ; N#Cc1cccc(-c2ccc3oc4ccccc4c3c2)c1-n1c2ccccc2c2ccc3sc4ccccc4c3c21 ; N#Cc1ccc2oc3ccc(-c4ccccc4-n4c5ccccc5c5ccc6sc7ccccc7c6c54)cc3c2c1 ; N#Cc1ccc2oc3ccc(-c4c(C#N)cccc4-n4c5ccccc5c5ccc6sc7ccccc7c6c54)cc3c2c1 ; N#Cc1ccc(-n2c3ccccc3c3ccc4sc5ccccc5c4c32)c(-c2ccc3oc4ccc(C#N)cc4c3c2)c1]

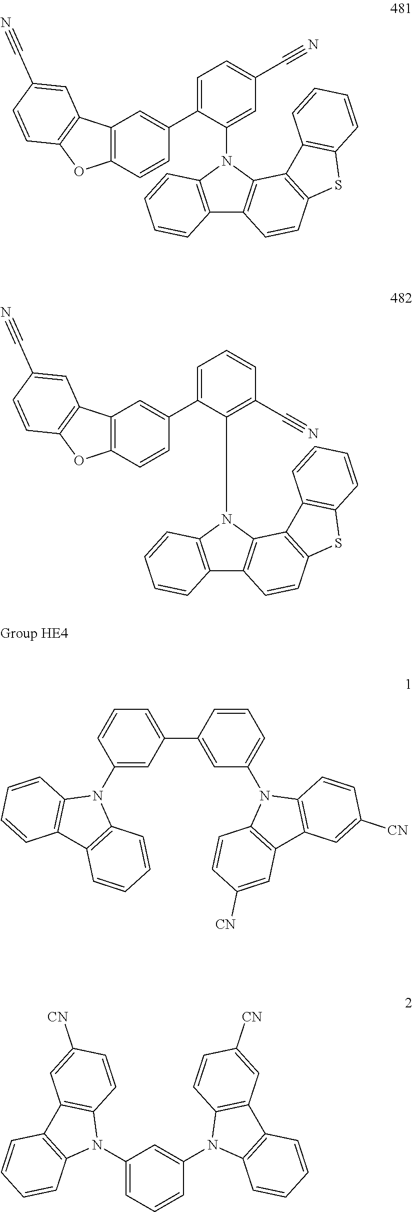

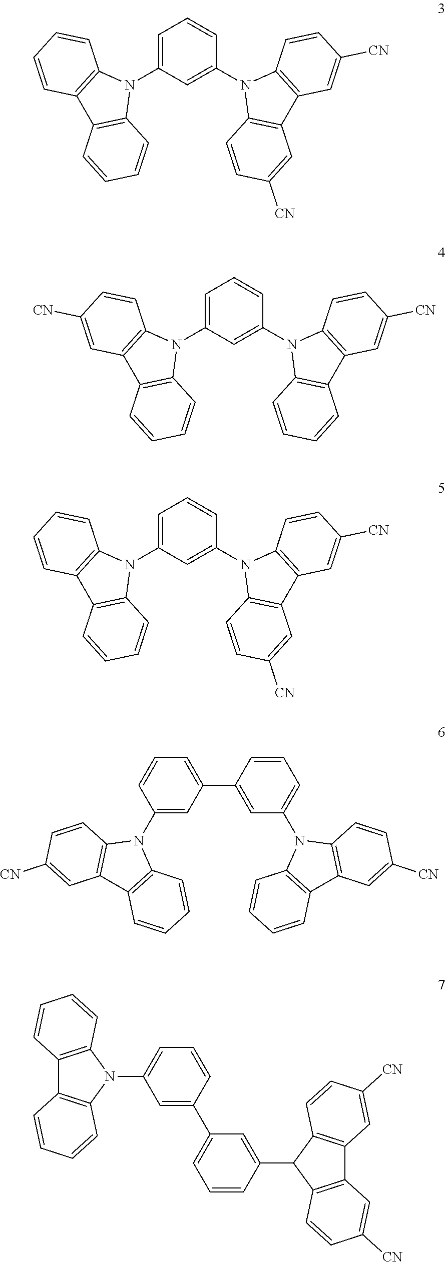

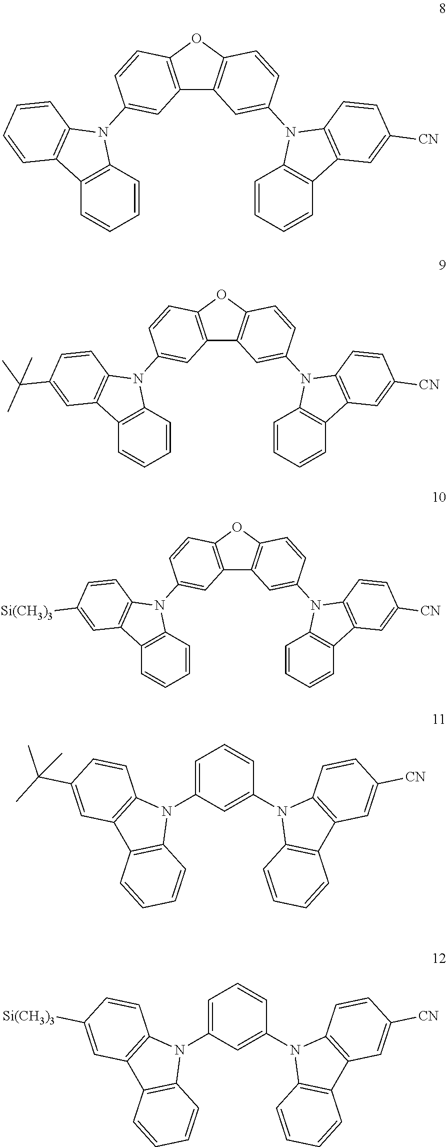

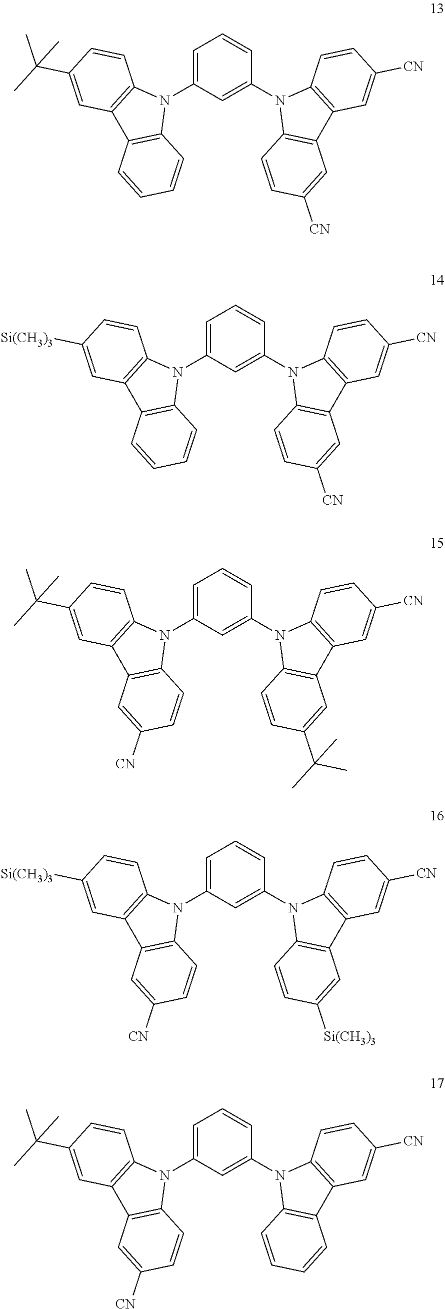

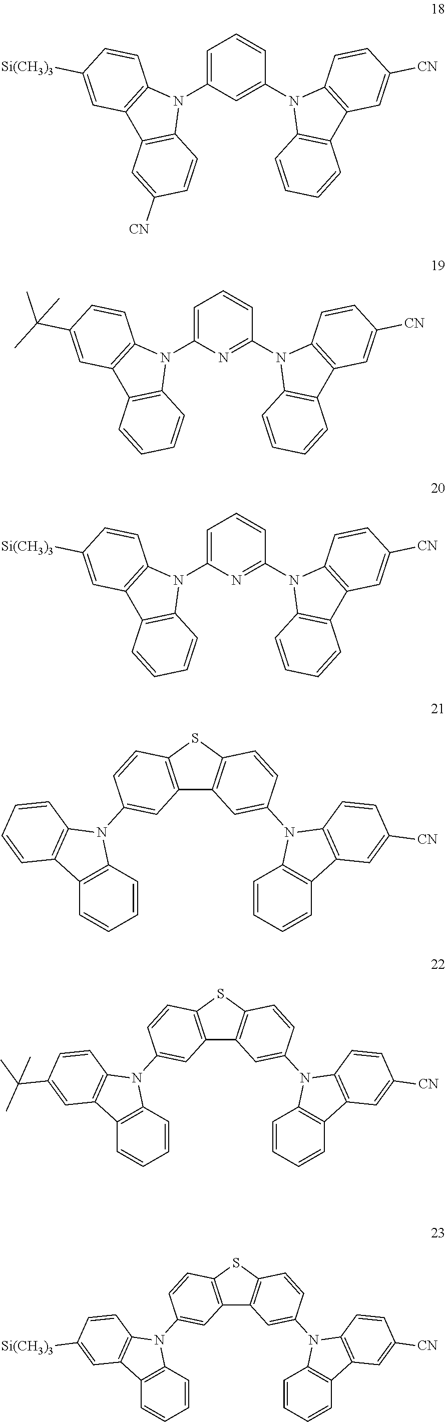

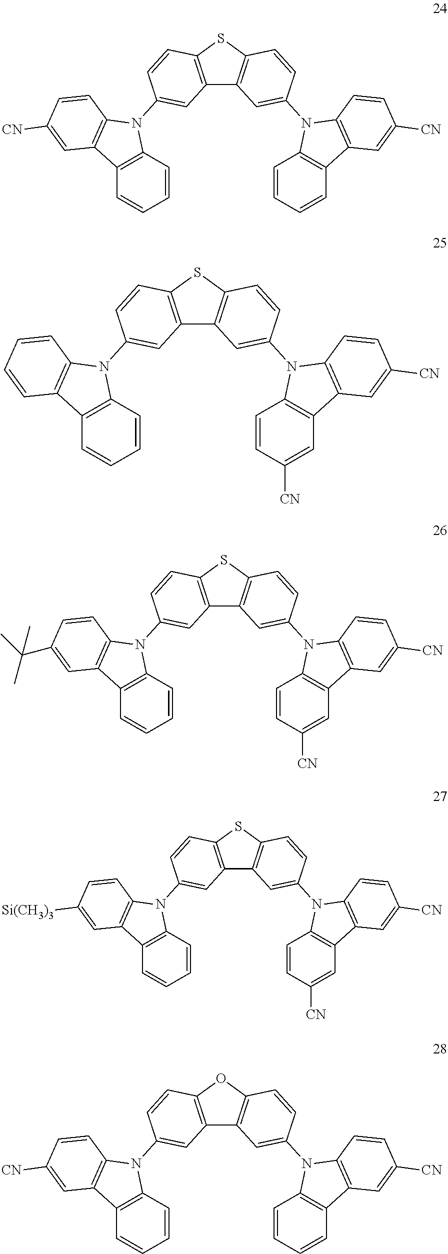

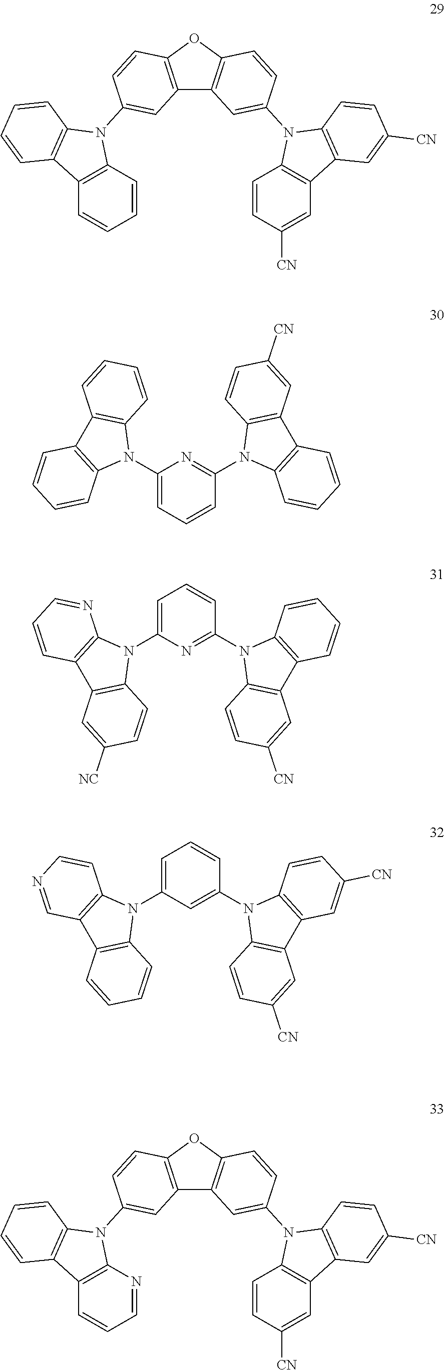 [SMILES: N#Cc1ccc2c(c1)c1cc(C#N)ccc1n2-c1ccc2oc3ccc(-n4c5ccccc5c5ccccc54)cc3c2c1 ; N#Cc1ccc2c(c1)c1ccccc1n2-c1cccc(-n2c3ccccc3c3ccccc32)n1 ; N#Cc1ccc2c(c1)c1ccccc1n2-c1cccc(-n2c3ccc(C#N)cc3c3cccnc32)n1 ; N#Cc1ccc2c(c1)c1cc(C#N)ccc1n2-c1cccc(-n2c3ccccc3c3cnccc32)c1 ; N#Cc1ccc2c(c1)c1cc(C#N)ccc1n2-c1ccc2oc3ccc(-n4c5ccccc5c5cccnc54)cc3c2c1]

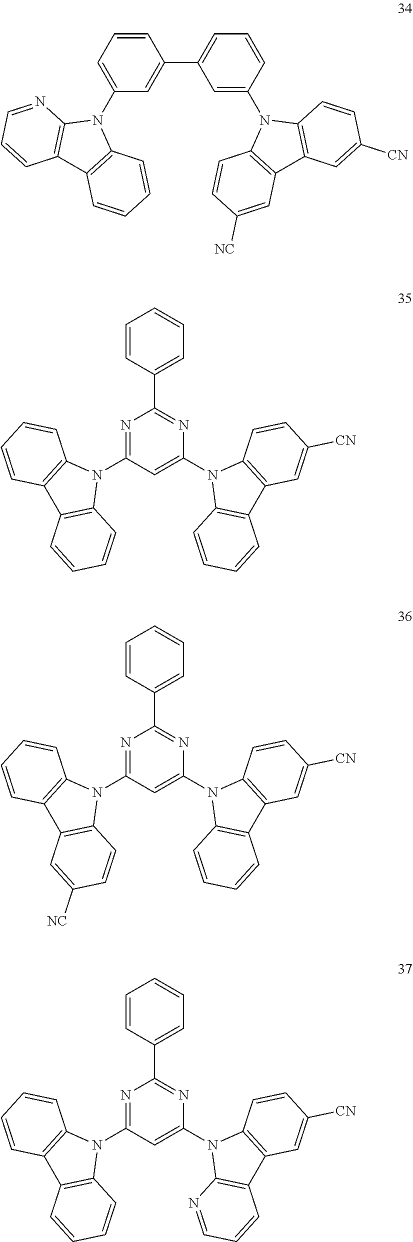 [SMILES: N#Cc1ccc2c(c1)c1cc(C#N)ccc1n2-c1cccc(-c2cccc(-n3c4ccccc4c4cccnc43)c2)c1 ; N#Cc1ccc2c(c1)c1ccccc1n2-c1cc(-n2c3ccccc3c3ccccc32)nc(-c2ccccc2)n1 ; N#Cc1ccc2c(c1)c1ccccc1n2-c1cc(-n2c3ccccc3c3cc(C#N)ccc32)nc(-c2ccccc2)n1 ; N#Cc1ccc2c(c1)c1cccnc1n2-c1cc(-n2c3ccccc3c3ccccc32)nc(-c2ccccc2)n1]

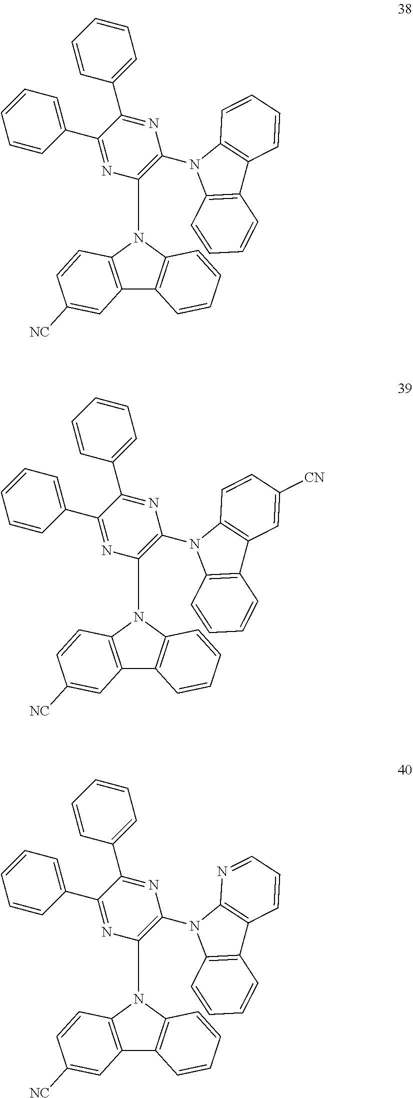 [SMILES: N#Cc1ccc2c(c1)c1ccccc1n2-c1nc(-c2ccccc2)c(-c2ccccc2)nc1-n1c2ccccc2c2ccccc21 ; N#Cc1ccc2c(c1)c1ccccc1n2-c1nc(-c2ccccc2)c(-c2ccccc2)nc1-n1c2ccccc2c2cc(C#N)ccc21 ; N#Cc1ccc2c(c1)c1ccccc1n2-c1nc(-c2ccccc2)c(-c2ccccc2)nc1-n1c2ccccc2c2cccnc21]

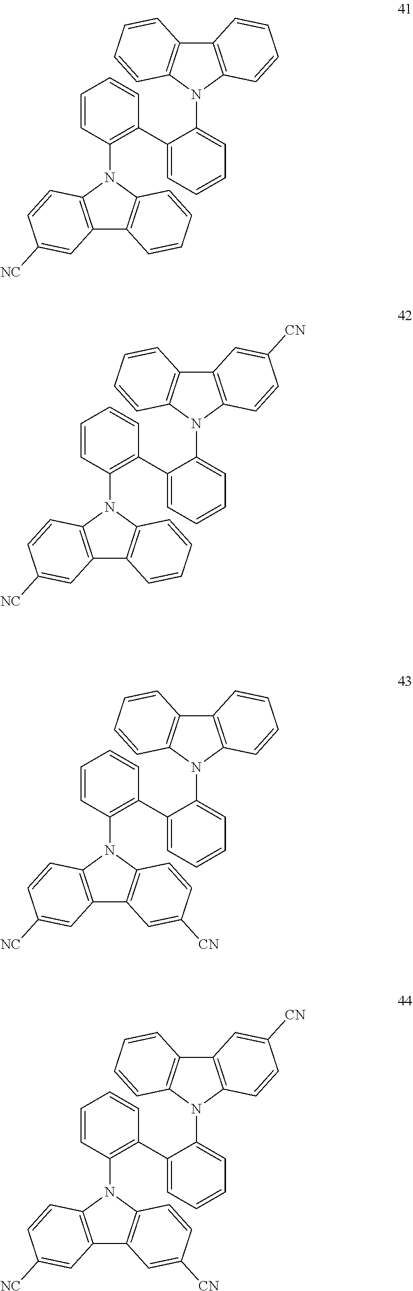 [SMILES: N#Cc1ccc2c(c1)c1ccccc1n2-c1ccccc1-c1ccccc1-n1c2ccccc2c2ccccc21 ; N#Cc1ccc2c(c1)c1ccccc1n2-c1ccccc1-c1ccccc1-n1c2ccccc2c2cc(C#N)ccc21 ; N#Cc1ccc2c(c1)c1cc(C#N)ccc1n2-c1ccccc1-c1ccccc1-n1c2ccccc2c2ccccc21 ; N#Cc1ccc2c(c1)c1ccccc1n2-c1ccccc1-c1ccccc1-n1c2ccc(C#N)cc2c2cc(C#N)ccc21]

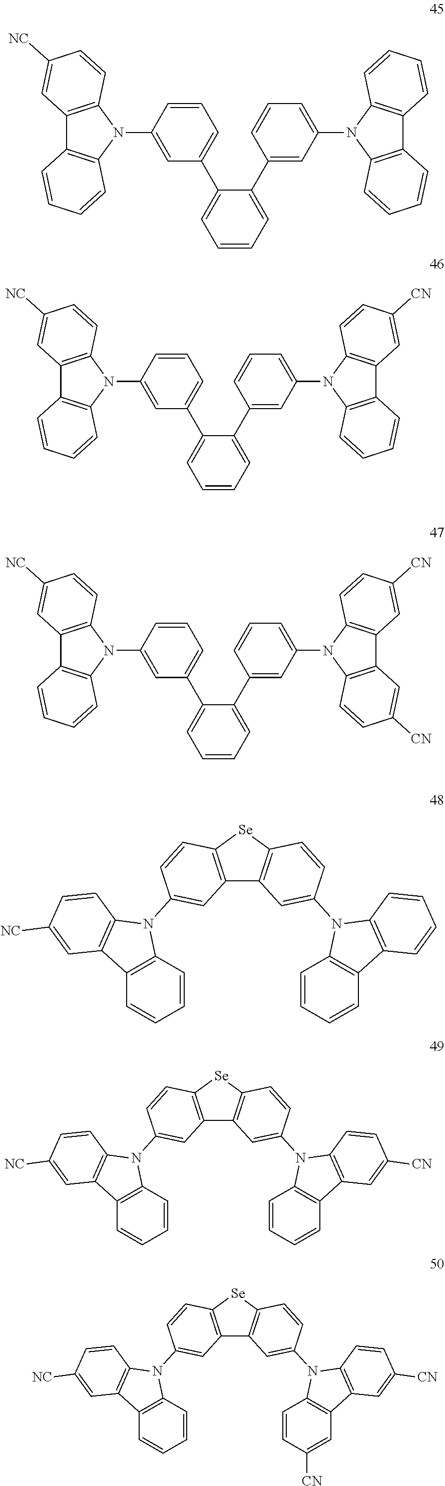 [SMILES: N#Cc1ccc2c(c1)c1ccccc1n2-c1cccc(-c2ccccc2-c2cccc(-n3c4ccccc4c4ccccc43)c2)c1 ; N#Cc1ccc2c(c1)c1ccccc1n2-c1cccc(-c2ccccc2-c2cccc(-n3c4ccccc4c4cc(C#N)ccc43)c2)c1 ; N#Cc1ccc2c(c1)c1ccccc1n2-c1cccc(-c2ccccc2-c2cccc(-n3c4ccc(C#N)cc4c4cc(C#N)ccc43)c2)c1 ; N#Cc1ccc2c(c1)c1ccccc1n2-c1ccc2[se]c3ccc(-n4c5ccccc5c5ccccc54)cc3c2c1 ; N#Cc1ccc2c(c1)c1ccccc1n2-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccc(C#N)cc5c5cc(C#N)ccc54)cc3c2c1]

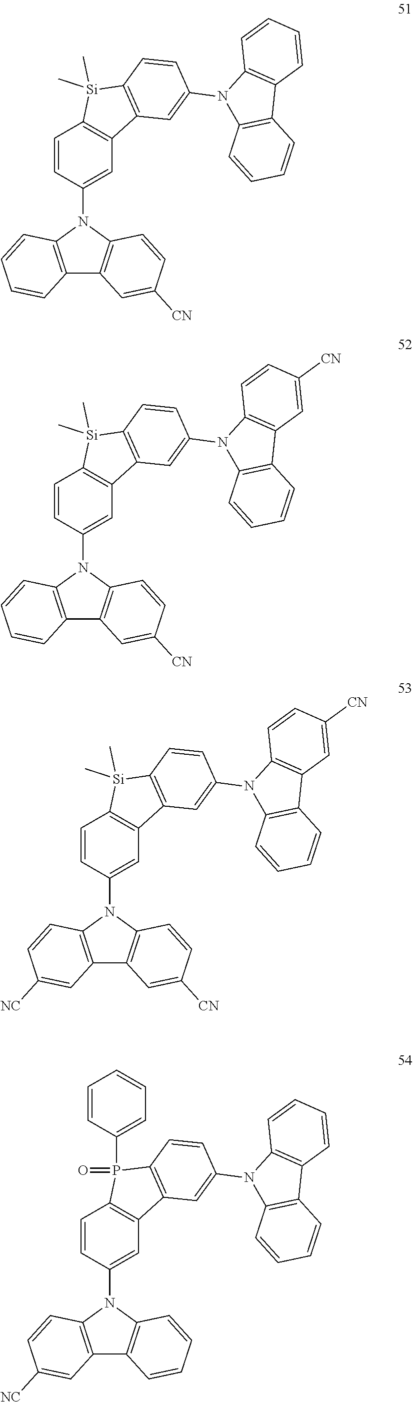 [SMILES: C[Si]1(C)c2ccc(-n3c4ccccc4c4ccccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccc(C#N)cc4c4cc(C#N)ccc43)ccc21 ; N#Cc1ccc2c(c1)c1ccccc1n2-c1ccc2c(c1)-c1cc(-n3c4ccccc4c4ccccc43)ccc1P2(=O)c1ccccc1]

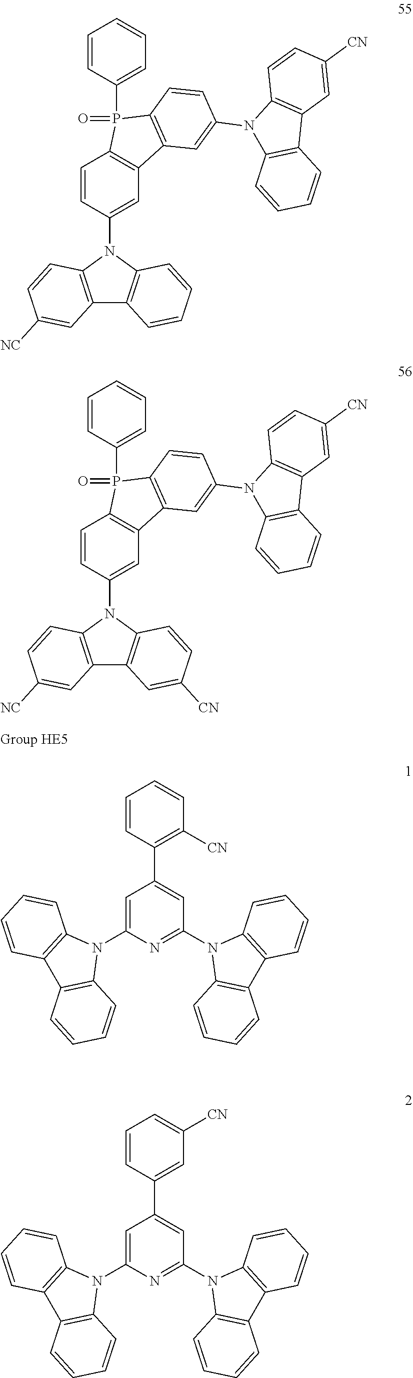 [SMILES: N#Cc1ccc2c(c1)c1ccccc1n2-c1ccc2c(c1)-c1cc(-n3c4ccccc4c4cc(C#N)ccc43)ccc1P2(=O)c1ccccc1 ; N#Cc1ccc2c(c1)c1ccccc1n2-c1ccc2c(c1)-c1cc(-n3c4ccc(C#N)cc4c4cc(C#N)ccc43)ccc1P2(=O)c1ccccc1 ; N#Cc1ccccc1-c1cc(-n2c3ccccc3c3ccccc32)nc(-n2c3ccccc3c3ccccc32)c1 ; N#Cc1cccc(-c2cc(-n3c4ccccc4c4ccccc43)nc(-n3c4ccccc4c4ccccc43)c2)c1]

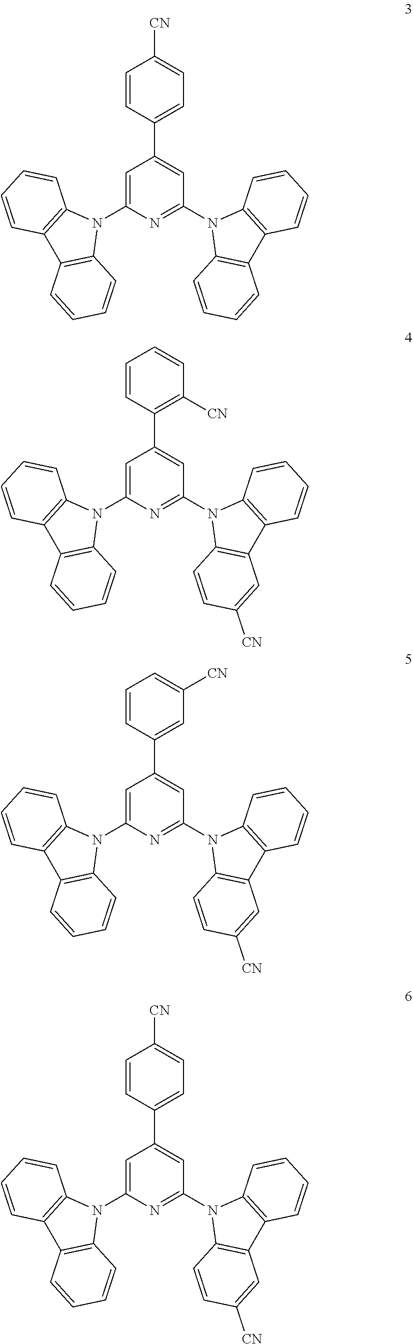

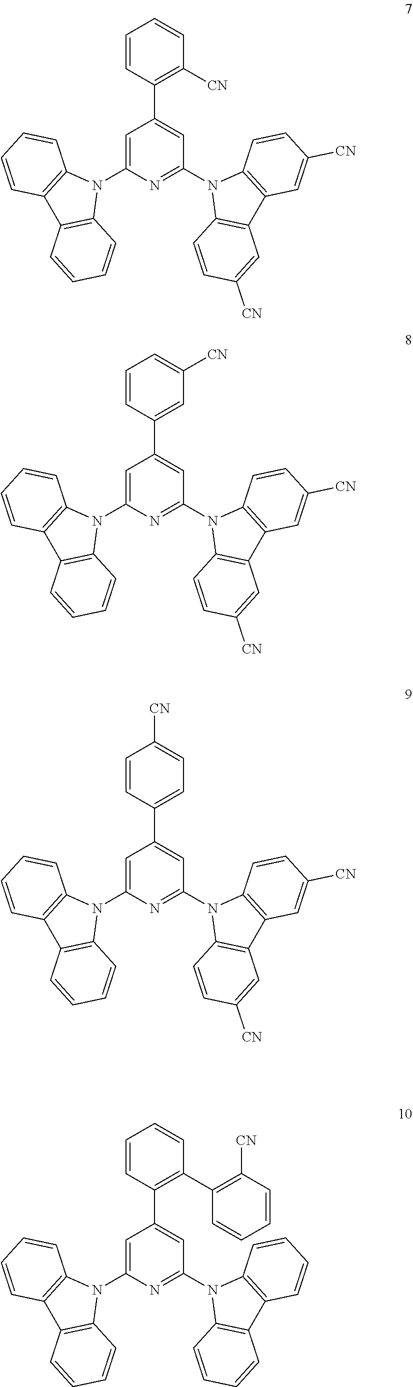 [SMILES: N#Cc1ccc2c(c1)c1cc(C#N)ccc1n2-c1cc(-c2ccccc2C#N)cc(-n2c3ccccc3c3ccccc32)n1 ; N#Cc1cccc(-c2cc(-n3c4ccccc4c4ccccc43)nc(-n3c4ccc(C#N)cc4c4cc(C#N)ccc43)c2)c1 ; N#Cc1ccc(-c2cc(-n3c4ccccc4c4ccccc43)nc(-n3c4ccc(C#N)cc4c4cc(C#N)ccc43)c2)cc1 ; N#Cc1ccccc1-c1ccccc1-c1cc(-n2c3ccccc3c3ccccc32)nc(-n2c3ccccc3c3ccccc32)c1]

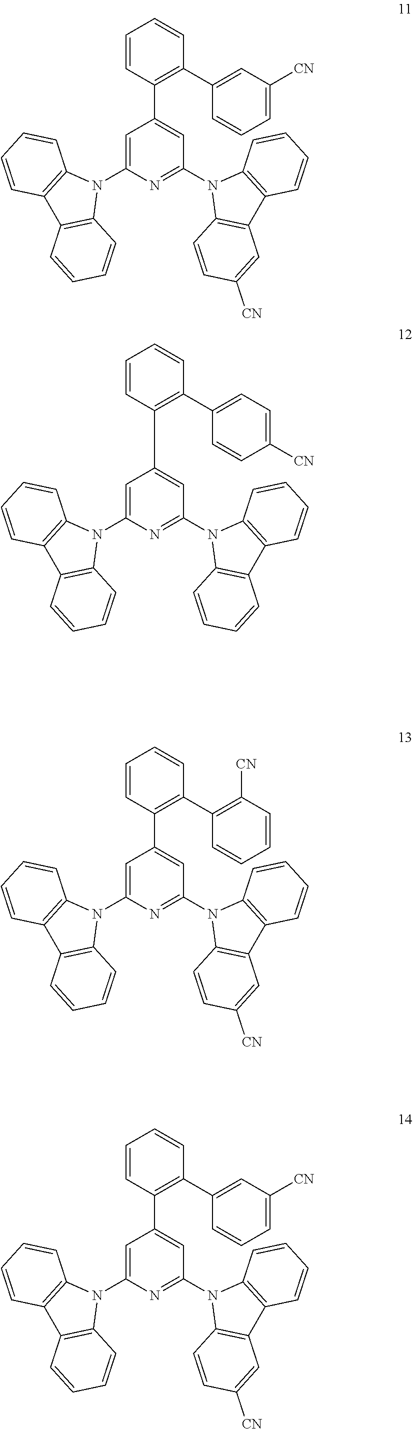 [SMILES: N#Cc1cccc(-c2ccccc2-c2cc(-n3c4ccccc4c4ccccc43)nc(-n3c4ccccc4c4cc(C#N)ccc43)c2)c1 ; N#Cc1ccc(-c2ccccc2-c2cc(-n3c4ccccc4c4ccccc43)nc(-n3c4ccccc4c4ccccc43)c2)cc1 ; N#Cc1ccc2c(c1)c1ccccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccccc4c4cc(C#N)ccc43)c2)c1]

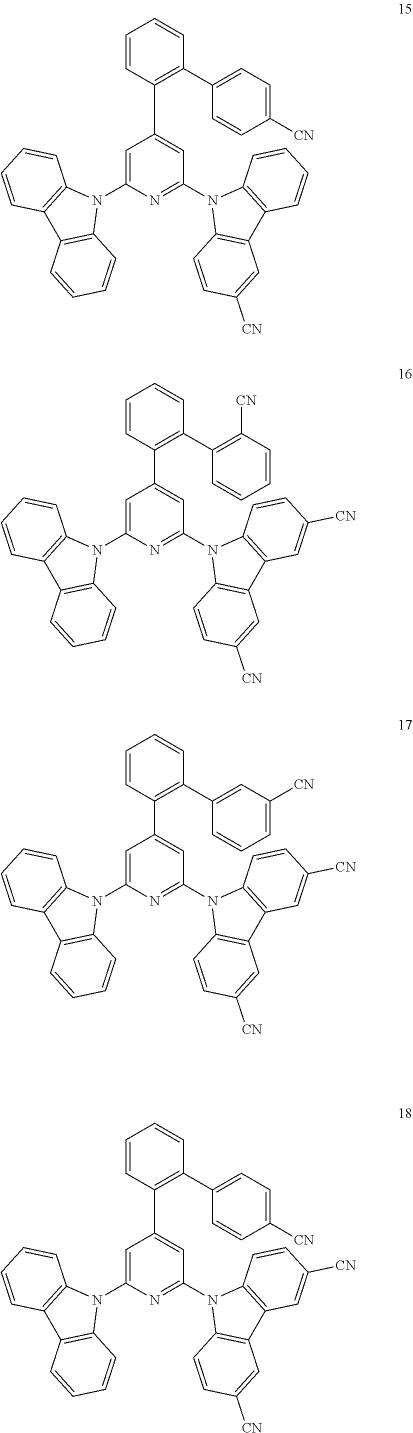 [SMILES: N#Cc1ccc(-c2ccccc2-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2cc(-n3c4ccccc4c4ccccc43)nc(-n3c4ccc(C#N)cc4c4cc(C#N)ccc43)c2)cc1]

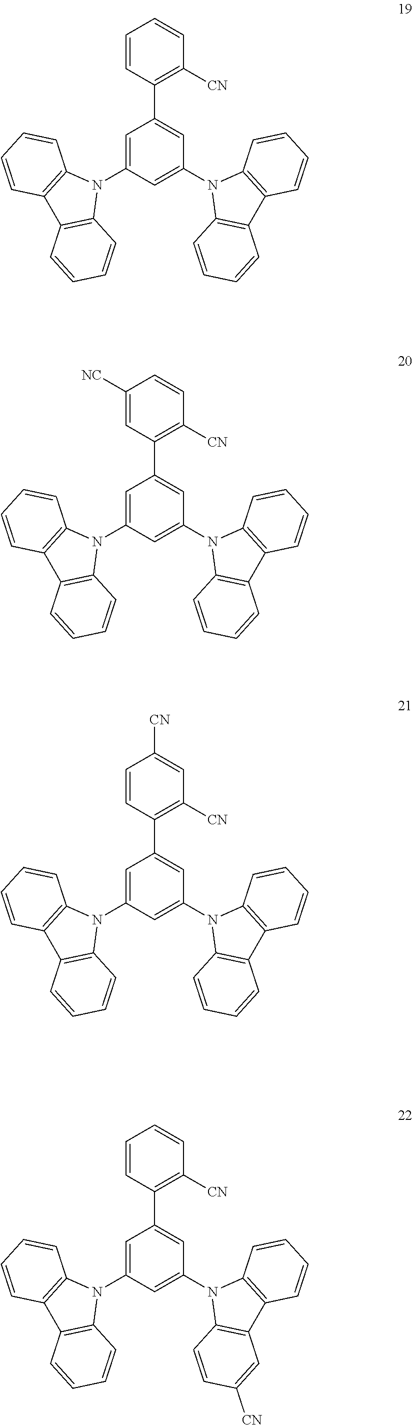 [SMILES: N#Cc1ccccc1-c1cc(-n2c3ccccc3c3ccccc32)cc(-n2c3ccccc3c3ccccc32)c1 ; N#Cc1ccc(C#N)c(-c2cc(-n3c4ccccc4c4ccccc43)cc(-n3c4ccccc4c4ccccc43)c2)c1 ; N#Cc1ccc(-c2cc(-n3c4ccccc4c4ccccc43)cc(-n3c4ccccc4c4ccccc43)c2)c(C#N)c1 ; N#Cc1ccc2c(c1)c1ccccc1n2-c1cc(-c2ccccc2C#N)cc(-n2c3ccccc3c3ccccc32)c1]

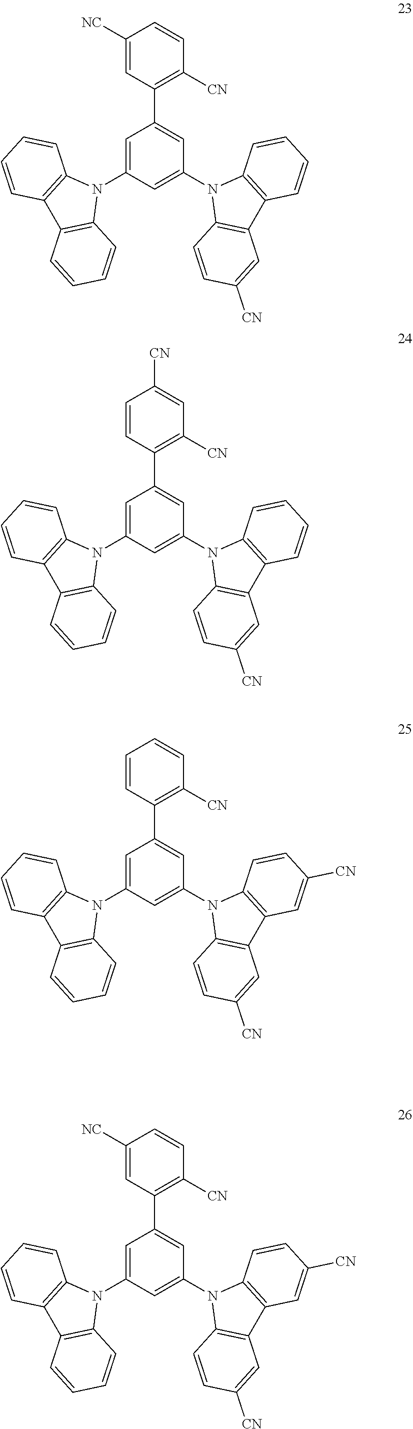 [SMILES: N#Cc1ccc(C#N)c(-c2cc(-n3c4ccccc4c4ccccc43)cc(-n3c4ccccc4c4cc(C#N)ccc43)c2)c1 ; N#Cc1ccc(-c2cc(-n3c4ccccc4c4ccccc43)cc(-n3c4ccccc4c4cc(C#N)ccc43)c2)c(C#N)c1 ; N#Cc1ccc2c(c1)c1cc(C#N)ccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccc(C#N)cc4c4cc(C#N)ccc43)c2)c1]

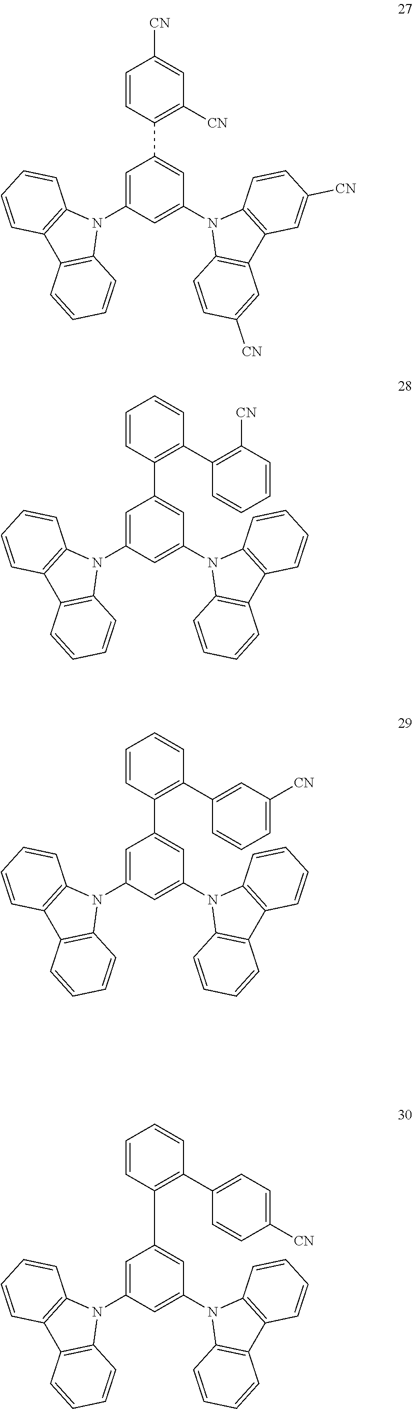

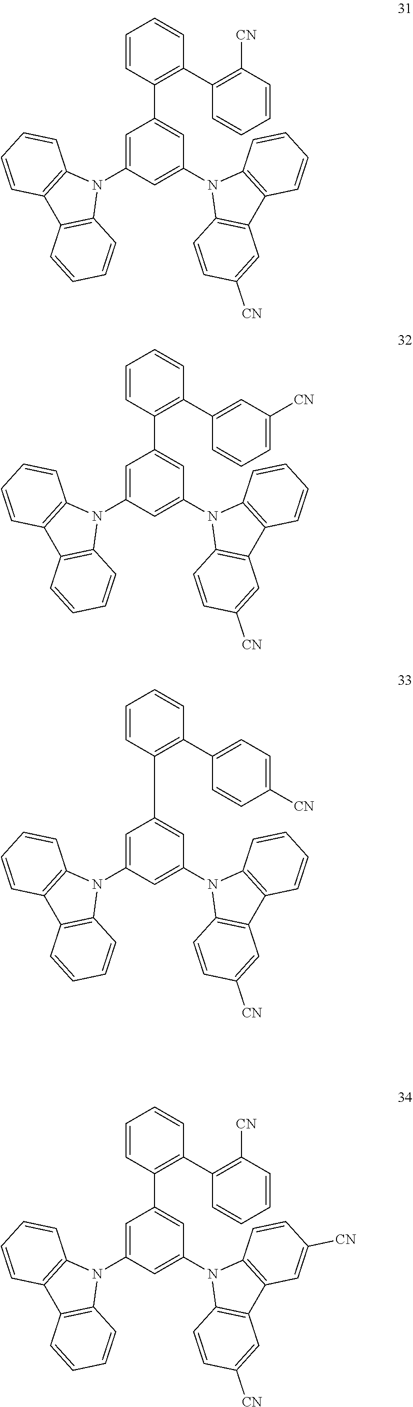 [SMILES: N#Cc1ccc2c(c1)c1ccccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccccc4c4cc(C#N)ccc43)c2)c1 ; N#Cc1ccc(-c2ccccc2-c2cc(-n3c4ccccc4c4ccccc43)cc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)c1]

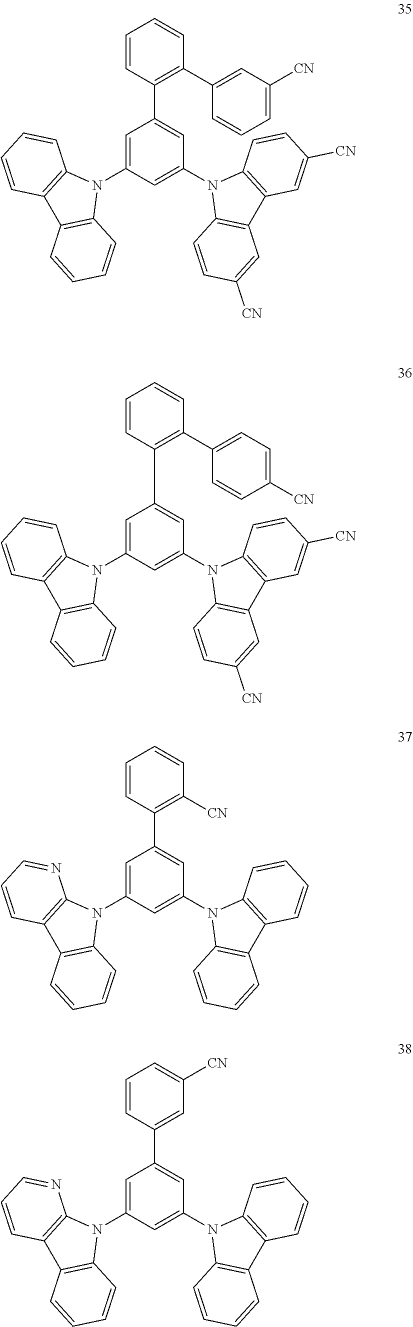 [SMILES: N#Cc1cccc(-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2cc(-n3c4ccccc4c4ccccc43)cc(-n3c4ccc(C#N)cc4c4cc(C#N)ccc43)c2)cc1 ; N#Cc1ccccc1-c1cc(-n2c3ccccc3c3ccccc32)cc(-n2c3ccccc3c3cccnc32)c1 ; N#Cc1cccc(-c2cc(-n3c4ccccc4c4ccccc43)cc(-n3c4ccccc4c4cccnc43)c2)c1]

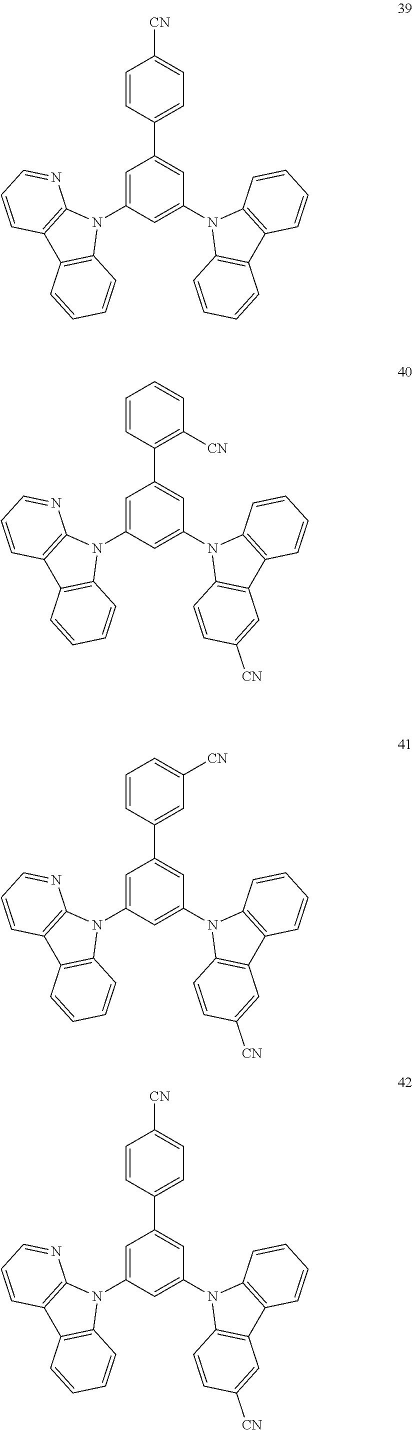 [SMILES: N#Cc1ccc(-c2cc(-n3c4ccccc4c4ccccc43)cc(-n3c4ccccc4c4cccnc43)c2)cc1 ; N#Cc1ccc2c(c1)c1ccccc1n2-c1cc(-c2ccccc2C#N)cc(-n2c3ccccc3c3cccnc32)c1 ; N#Cc1cccc(-c2cc(-n3c4ccccc4c4cc(C#N)ccc43)cc(-n3c4ccccc4c4cccnc43)c2)c1 ; N#Cc1ccc(-c2cc(-n3c4ccccc4c4cc(C#N)ccc43)cc(-n3c4ccccc4c4cccnc43)c2)cc1]

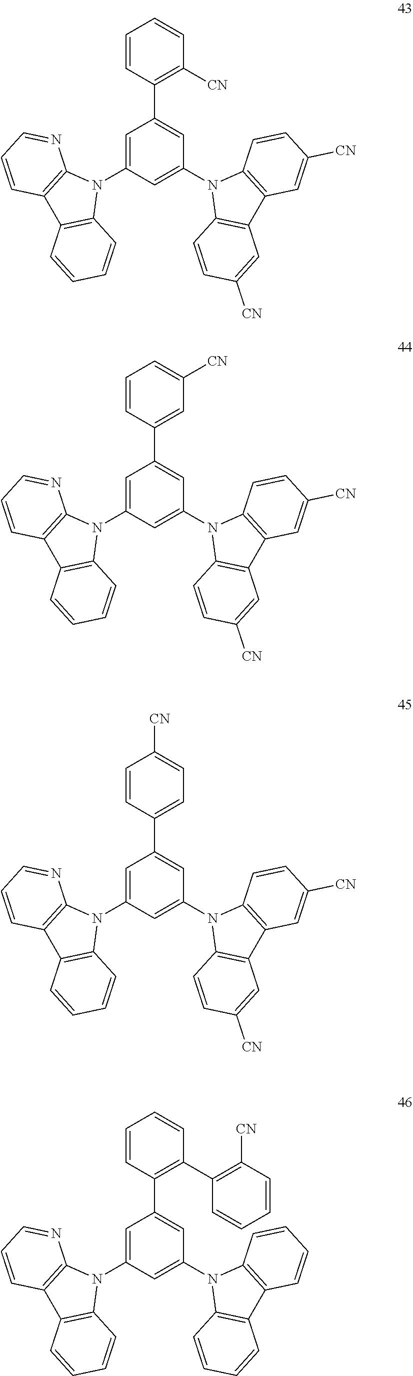 [SMILES: N#Cc1ccc2c(c1)c1cc(C#N)ccc1n2-c1cc(-c2ccccc2C#N)cc(-n2c3ccccc3c3cccnc32)c1 ; N#Cc1cccc(-c2cc(-n3c4ccc(C#N)cc4c4cc(C#N)ccc43)cc(-n3c4ccccc4c4cccnc43)c2)c1 ; N#Cc1ccc(-c2cc(-n3c4ccc(C#N)cc4c4cc(C#N)ccc43)cc(-n3c4ccccc4c4cccnc43)c2)cc1 ; N#Cc1ccccc1-c1ccccc1-c1cc(-n2c3ccccc3c3ccccc32)cc(-n2c3ccccc3c3cccnc32)c1]

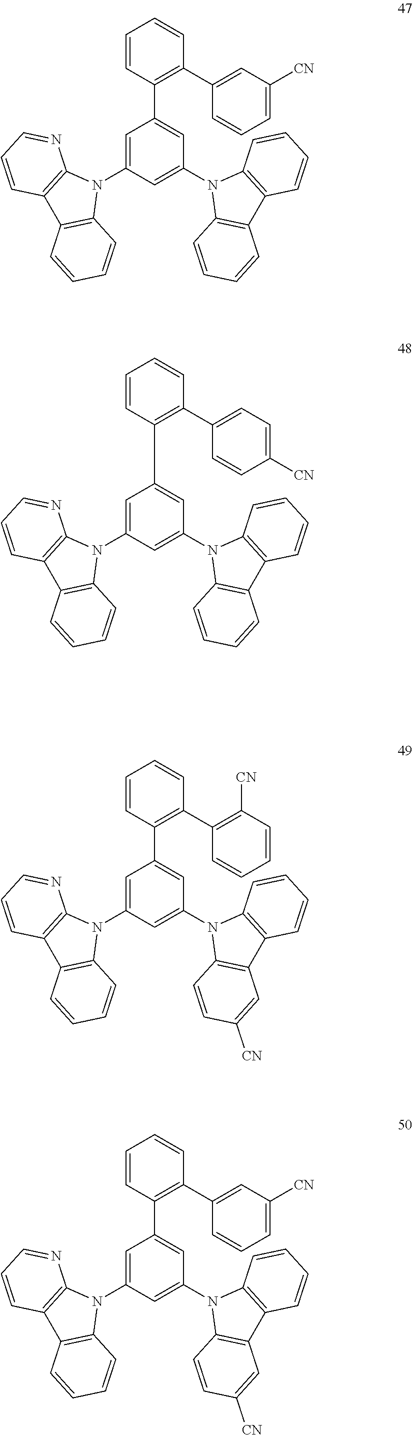 [SMILES: N#Cc1cccc(-c2ccccc2-c2cc(-n3c4ccccc4c4ccccc43)cc(-n3c4ccccc4c4cccnc43)c2)c1 ; N#Cc1ccc(-c2ccccc2-c2cc(-n3c4ccccc4c4ccccc43)cc(-n3c4ccccc4c4cccnc43)c2)cc1 ; N#Cc1ccc2c(c1)c1ccccc1n2-c1cc(-c2ccccc2-c2ccccc2C#N)cc(-n2c3ccccc3c3cccnc32)c1 ; N#Cc1cccc(-c2ccccc2-c2cc(-n3c4ccccc4c4cc(C#N)ccc43)cc(-n3c4ccccc4c4cccnc43)c2)c1]

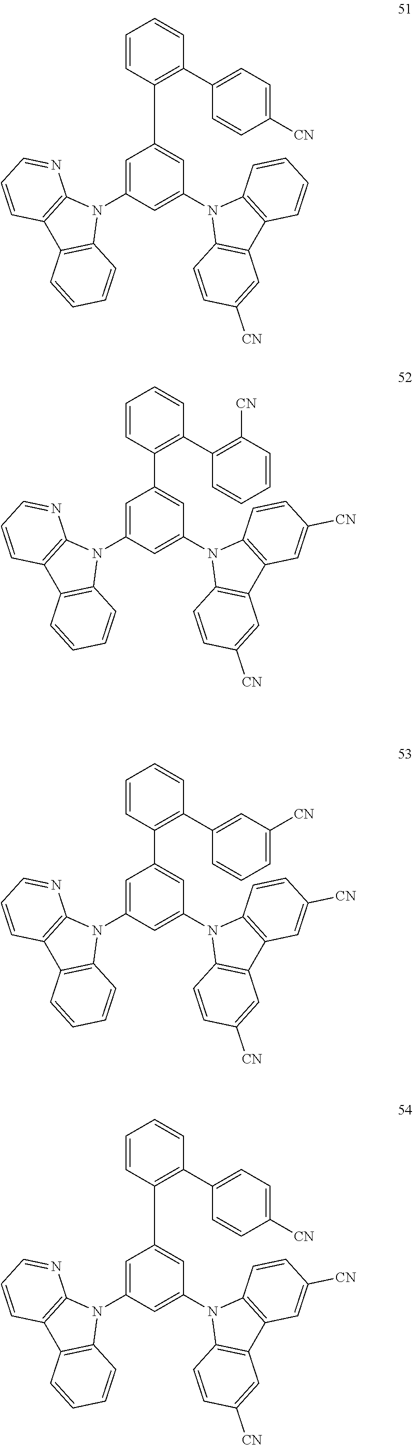 [SMILES: N#Cc1ccc(-c2ccccc2-c2cc(-n3c4ccccc4c4cc(C#N)ccc43)cc(-n3c4ccccc4c4cccnc43)c2)cc1 ; N#Cc1ccc2c(c1)c1cc(C#N)ccc1n2-c1cc(-c2ccccc2-c2ccccc2C#N)cc(-n2c3ccccc3c3cccnc32)c1 ; N#Cc1cccc(-c2ccccc2-c2cc(-n3c4ccc(C#N)cc4c4cc(C#N)ccc43)cc(-n3c4ccccc4c4cccnc43)c2)c1 ; N#Cc1ccc(-c2ccccc2-c2cc(-n3c4ccc(C#N)cc4c4cc(C#N)ccc43)cc(-n3c4ccccc4c4cccnc43)c2)cc1]

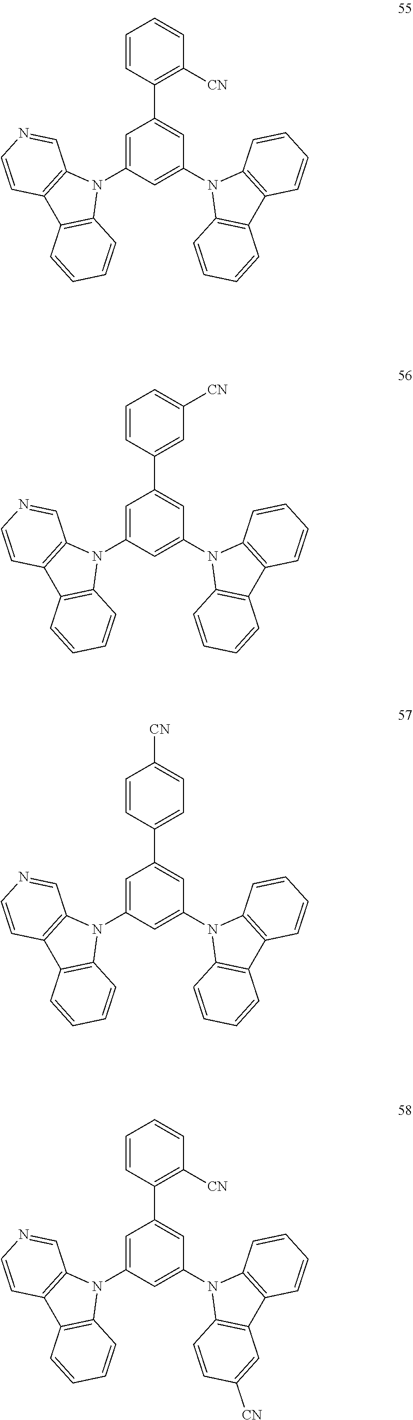 [SMILES: N#Cc1ccccc1-c1cc(-n2c3ccccc3c3ccccc32)cc(-n2c3ccccc3c3ccncc32)c1 ; N#Cc1cccc(-c2cc(-n3c4ccccc4c4ccccc43)cc(-n3c4ccccc4c4ccncc43)c2)c1 ; N#Cc1ccc(-c2cc(-n3c4ccccc4c4ccccc43)cc(-n3c4ccccc4c4ccncc43)c2)cc1 ; N#Cc1ccc2c(c1)c1ccccc1n2-c1cc(-c2ccccc2C#N)cc(-n2c3ccccc3c3ccncc32)c1]

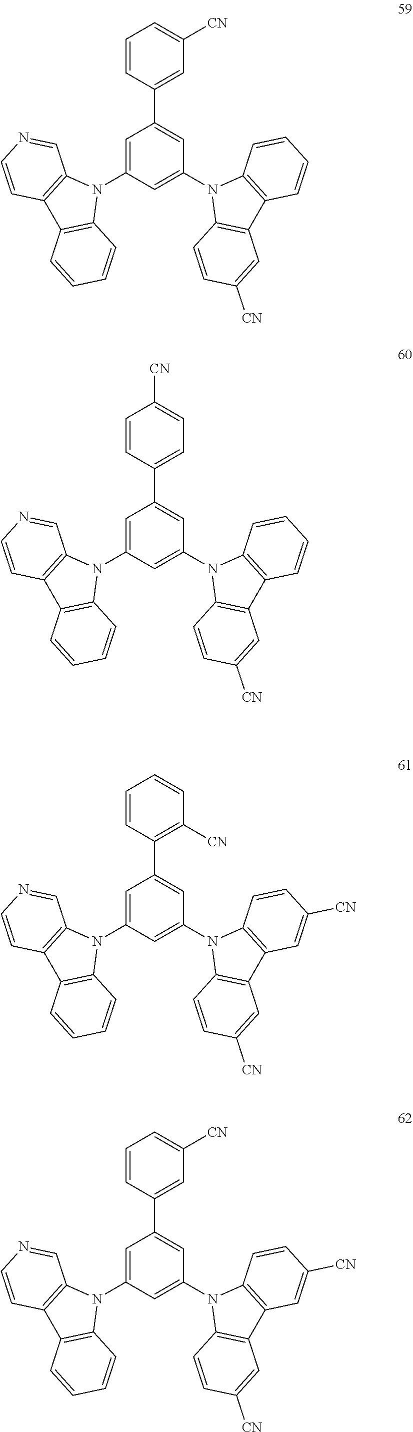 [SMILES: N#Cc1cccc(-c2cc(-n3c4ccccc4c4cc(C#N)ccc43)cc(-n3c4ccccc4c4ccncc43)c2)c1 ; N#Cc1ccc(-c2cc(-n3c4ccccc4c4cc(C#N)ccc43)cc(-n3c4ccccc4c4ccncc43)c2)cc1 ; N#Cc1ccc2c(c1)c1cc(C#N)ccc1n2-c1cc(-c2ccccc2C#N)cc(-n2c3ccccc3c3ccncc32)c1 ; N#Cc1cccc(-c2cc(-n3c4ccc(C#N)cc4c4cc(C#N)ccc43)cc(-n3c4ccccc4c4ccncc43)c2)c1]

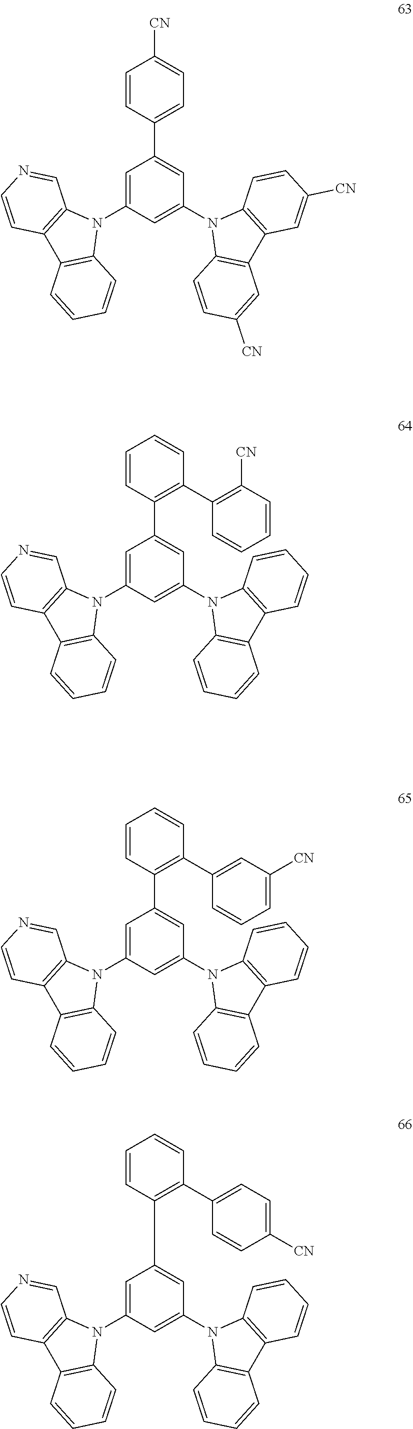 [SMILES: N#Cc1ccc(-c2cc(-n3c4ccc(C#N)cc4c4cc(C#N)ccc43)cc(-n3c4ccccc4c4ccncc43)c2)cc1 ; N#Cc1ccccc1-c1ccccc1-c1cc(-n2c3ccccc3c3ccccc32)cc(-n2c3ccccc3c3ccncc32)c1 ; N#Cc1cccc(-c2ccccc2-c2cc(-n3c4ccccc4c4ccccc43)cc(-n3c4ccccc4c4ccncc43)c2)c1 ; N#Cc1ccc(-c2ccccc2-c2cc(-n3c4ccccc4c4ccccc43)cc(-n3c4ccccc4c4ccncc43)c2)cc1]

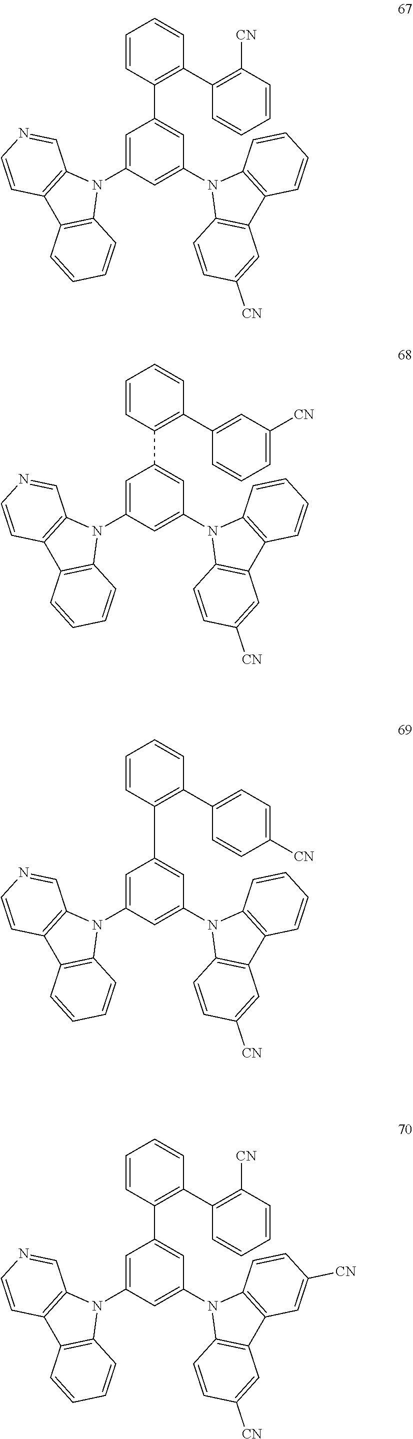

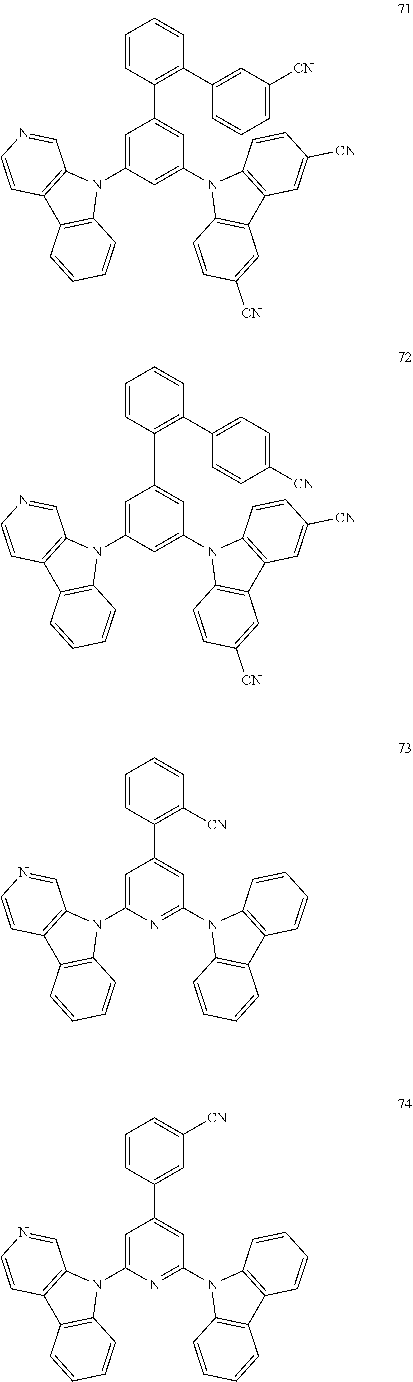 [SMILES: N#Cc1cccc(-c2ccccc2-c2cc(-n3c4ccc(C#N)cc4c4cc(C#N)ccc43)cc(-n3c4ccccc4c4ccncc43)c2)c1 ; N#Cc1ccc(-c2ccccc2-c2cc(-n3c4ccc(C#N)cc4c4cc(C#N)ccc43)cc(-n3c4ccccc4c4ccncc43)c2)cc1 ; N#Cc1ccccc1-c1cc(-n2c3ccccc3c3ccccc32)nc(-n2c3ccccc3c3ccncc32)c1 ; N#Cc1cccc(-c2cc(-n3c4ccccc4c4ccccc43)nc(-n3c4ccccc4c4ccncc43)c2)c1]

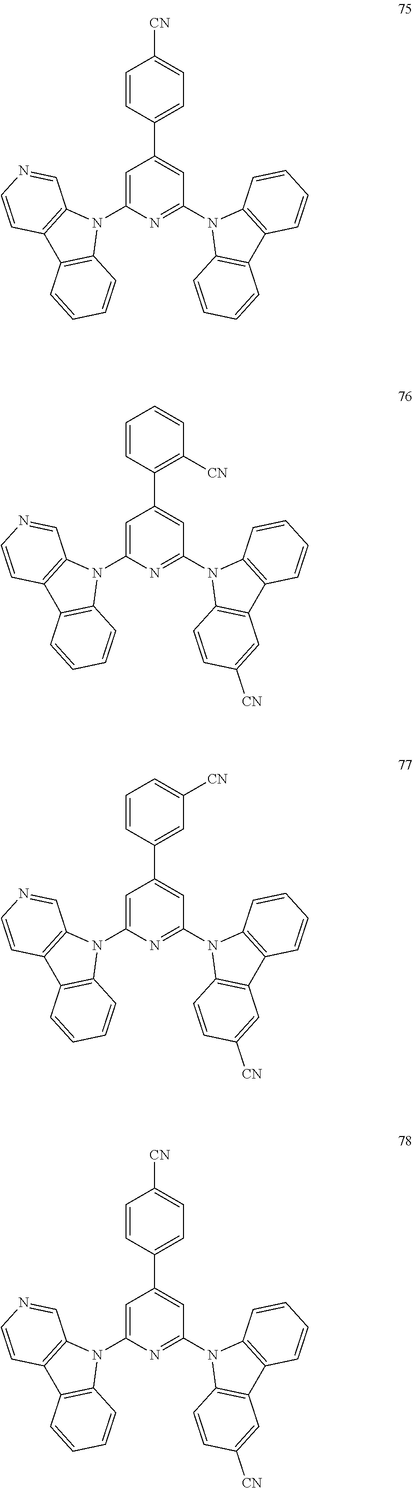 [SMILES: N#Cc1ccc(-c2cc(-n3c4ccccc4c4ccccc43)nc(-n3c4ccccc4c4ccncc43)c2)cc1 ; N#Cc1ccc2c(c1)c1ccccc1n2-c1cc(-c2ccccc2C#N)cc(-n2c3ccccc3c3ccncc32)n1 ; N#Cc1cccc(-c2cc(-n3c4ccccc4c4cc(C#N)ccc43)nc(-n3c4ccccc4c4ccncc43)c2)c1 ; N#Cc1ccc(-c2cc(-n3c4ccccc4c4cc(C#N)ccc43)nc(-n3c4ccccc4c4ccncc43)c2)cc1]

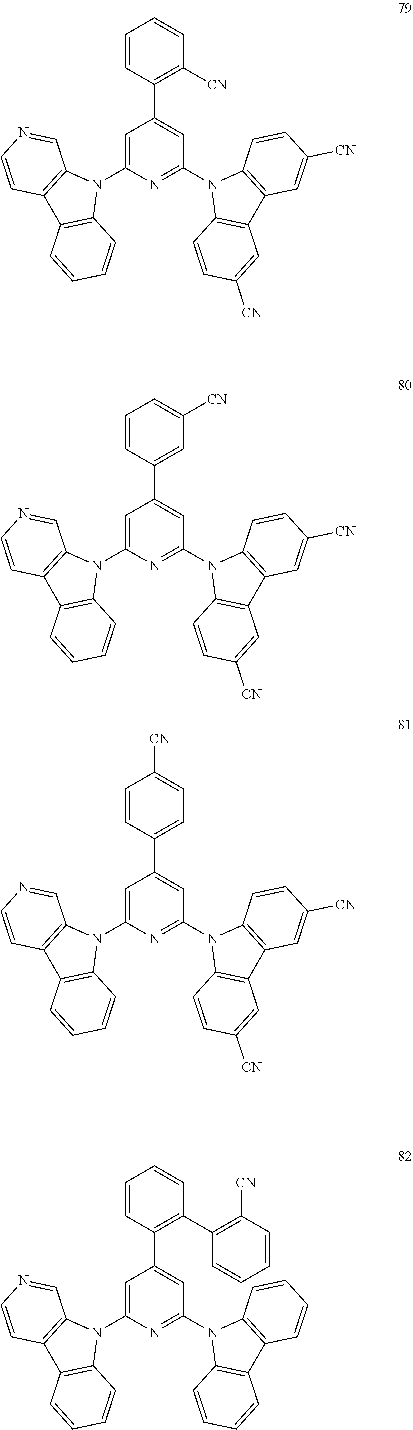 [SMILES: N#Cc1ccc2c(c1)c1cc(C#N)ccc1n2-c1cc(-c2ccccc2C#N)cc(-n2c3ccccc3c3ccncc32)n1 ; N#Cc1cccc(-c2cc(-n3c4ccc(C#N)cc4c4cc(C#N)ccc43)nc(-n3c4ccccc4c4ccncc43)c2)c1 ; N#Cc1ccc(-c2cc(-n3c4ccc(C#N)cc4c4cc(C#N)ccc43)nc(-n3c4ccccc4c4ccncc43)c2)cc1 ; N#Cc1ccccc1-c1ccccc1-c1cc(-n2c3ccccc3c3ccccc32)nc(-n2c3ccccc3c3ccncc32)c1]

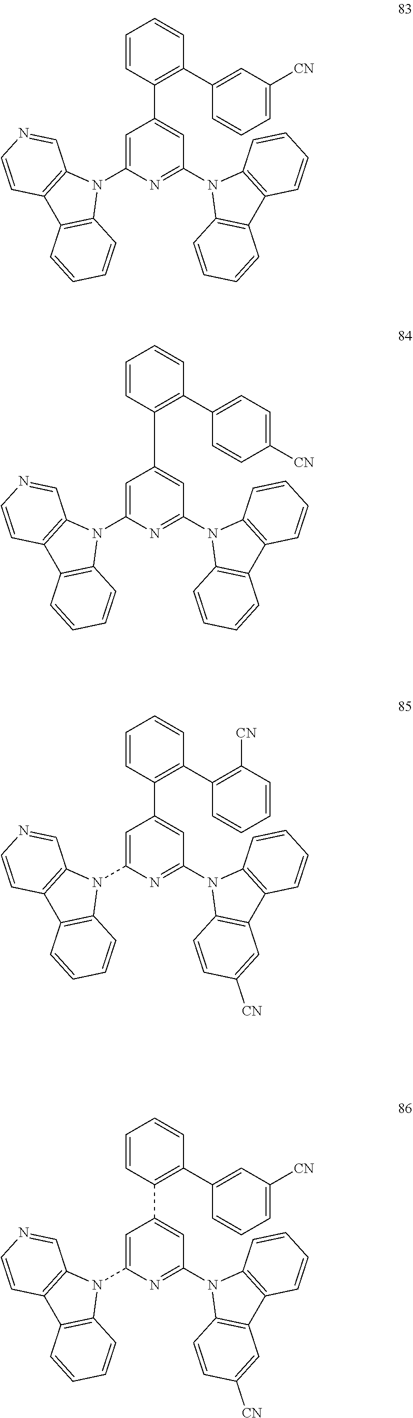

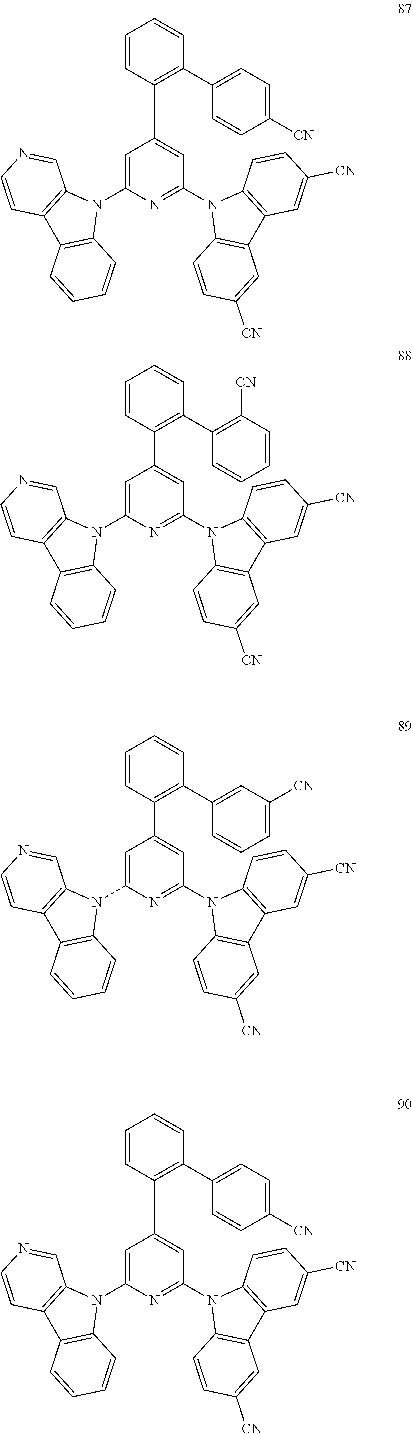

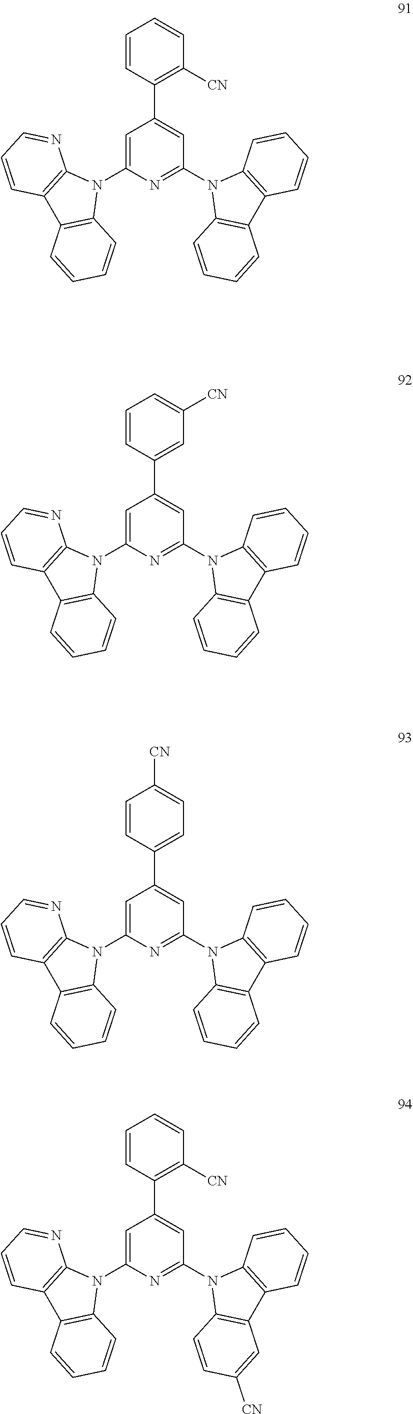 [SMILES: N#Cc1ccccc1-c1cc(-n2c3ccccc3c3ccccc32)nc(-n2c3ccccc3c3cccnc32)c1 ; N#Cc1cccc(-c2cc(-n3c4ccccc4c4ccccc43)nc(-n3c4ccccc4c4cccnc43)c2)c1 ; N#Cc1ccc(-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2C#N)cc(-n2c3ccccc3c3cccnc32)n1]

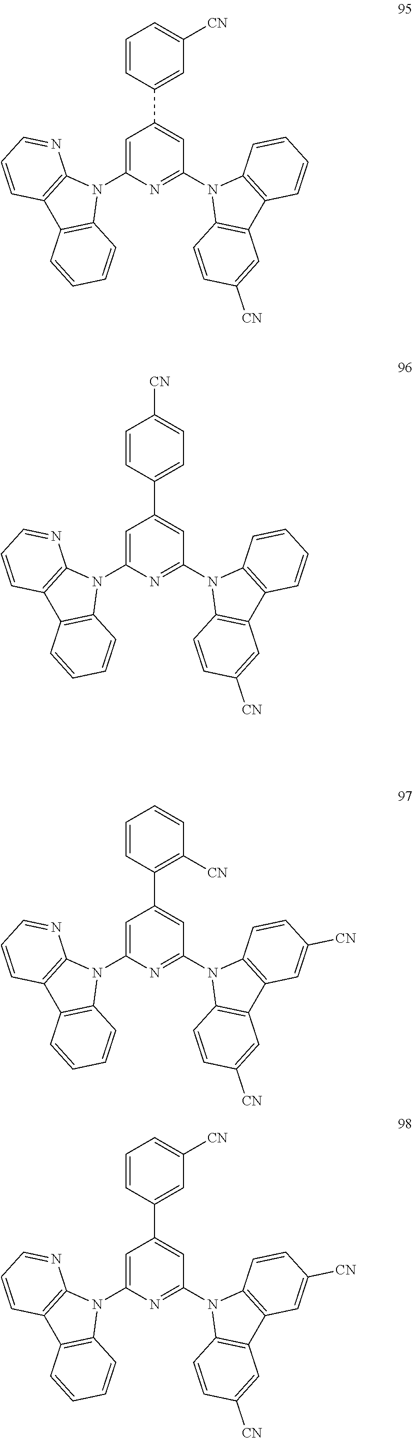

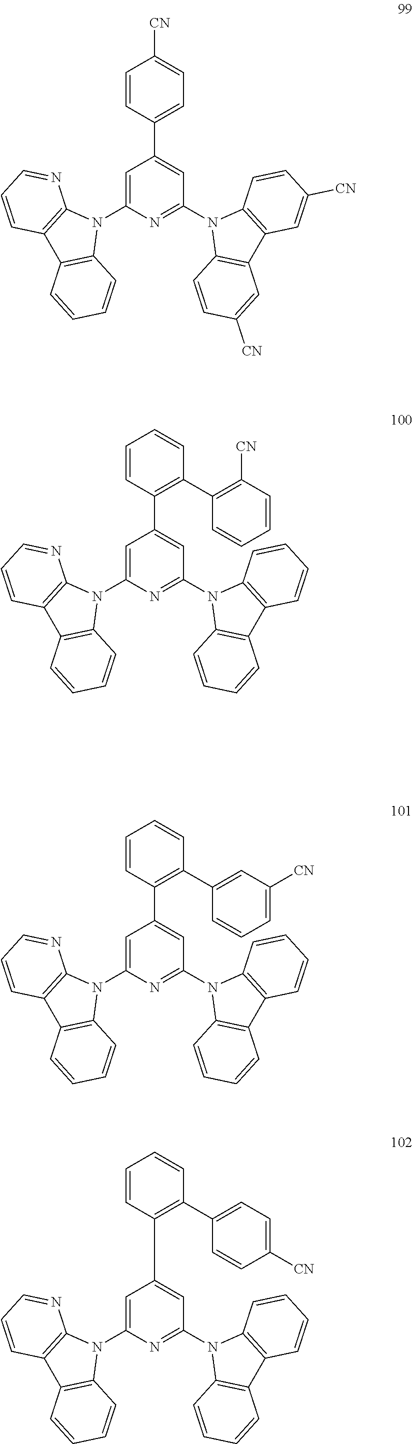 [SMILES: N#Cc1ccc(-c2cc(-n3c4ccc(C#N)cc4c4cc(C#N)ccc43)nc(-n3c4ccccc4c4cccnc43)c2)cc1 ; N#Cc1ccccc1-c1ccccc1-c1cc(-n2c3ccccc3c3ccccc32)nc(-n2c3ccccc3c3cccnc32)c1 ; N#Cc1cccc(-c2ccccc2-c2cc(-n3c4ccccc4c4ccccc43)nc(-n3c4ccccc4c4cccnc43)c2)c1 ; N#Cc1ccc(-c2ccccc2-c2cc(-n3c4ccccc4c4ccccc43)nc(-n3c4ccccc4c4cccnc43)c2)cc1]

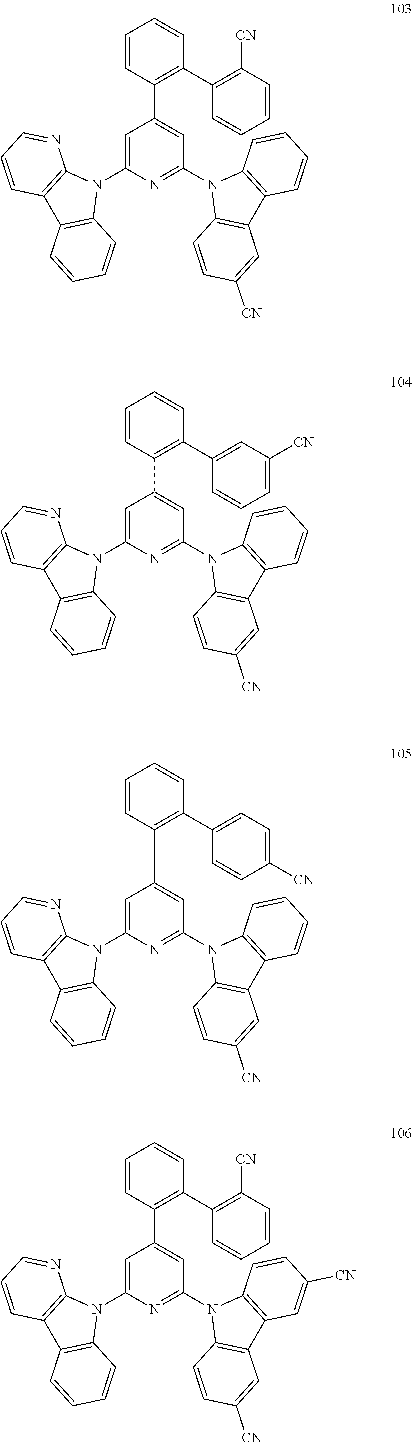

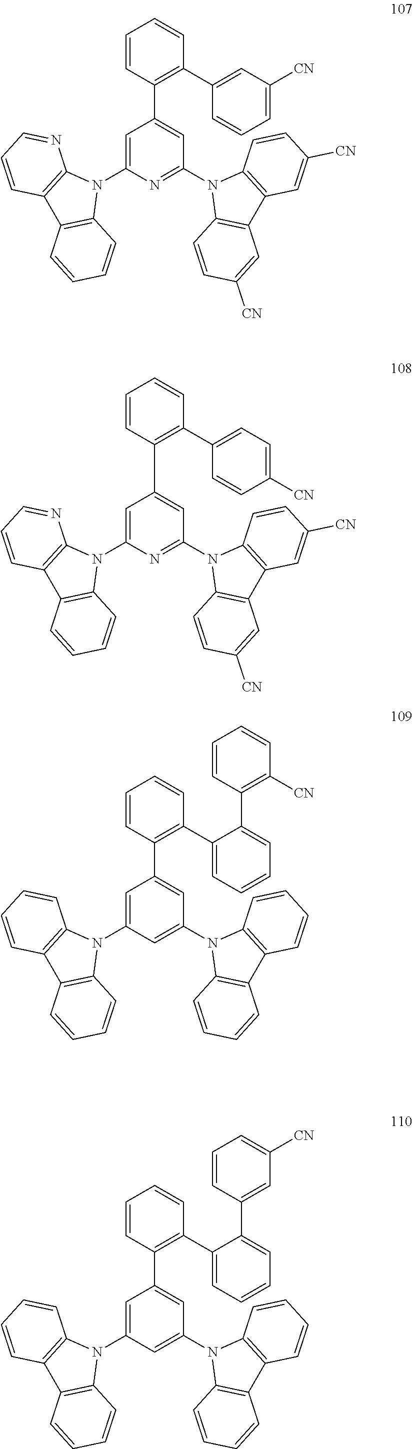

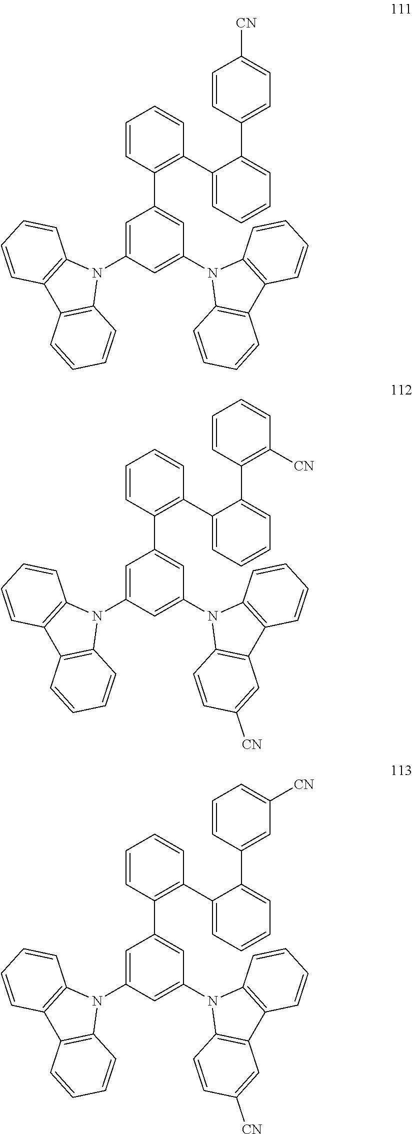

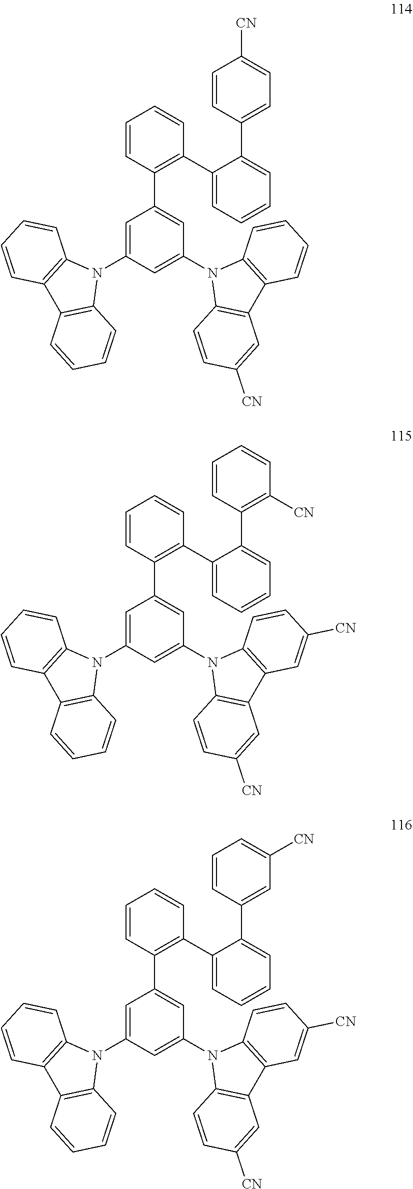

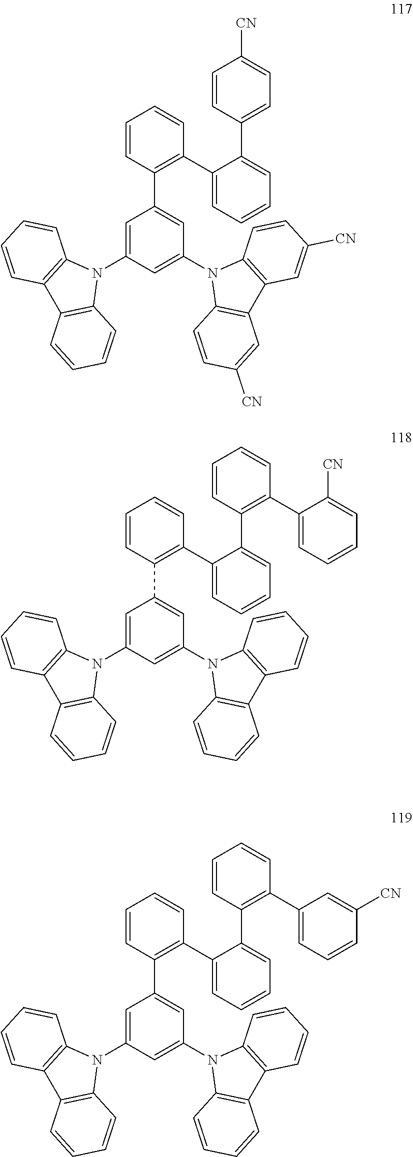

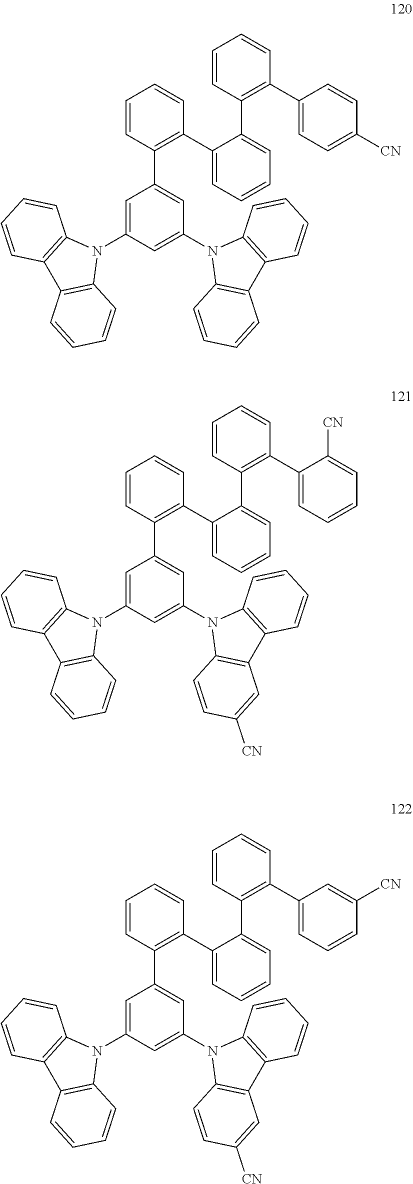 [SMILES: N#Cc1ccc(-c2ccccc2-c2ccccc2-c2ccccc2-c2cc(-n3c4ccccc4c4ccccc43)cc(-n3c4ccccc4c4ccccc43)c2)cc1 ; N#Cc1ccc2c(c1)c1ccccc1n2-c1cc(-c2ccccc2-c2ccccc2-c2ccccc2-c2ccccc2C#N)cc(-n2c3ccccc3c3ccccc32)c1 ; N#Cc1cccc(-c2ccccc2-c2ccccc2-c2ccccc2-c2cc(-n3c4ccccc4c4ccccc43)cc(-n3c4ccccc4c4cc(C#N)ccc43)c2)c1]

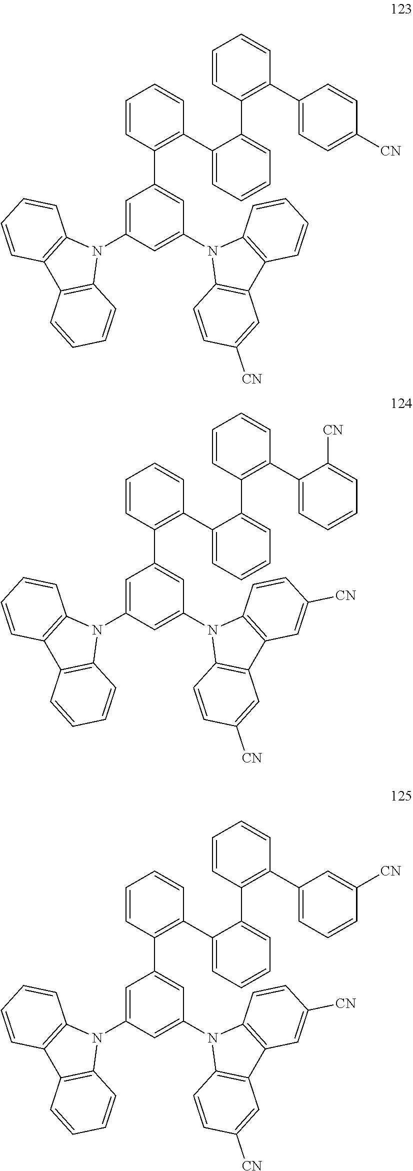

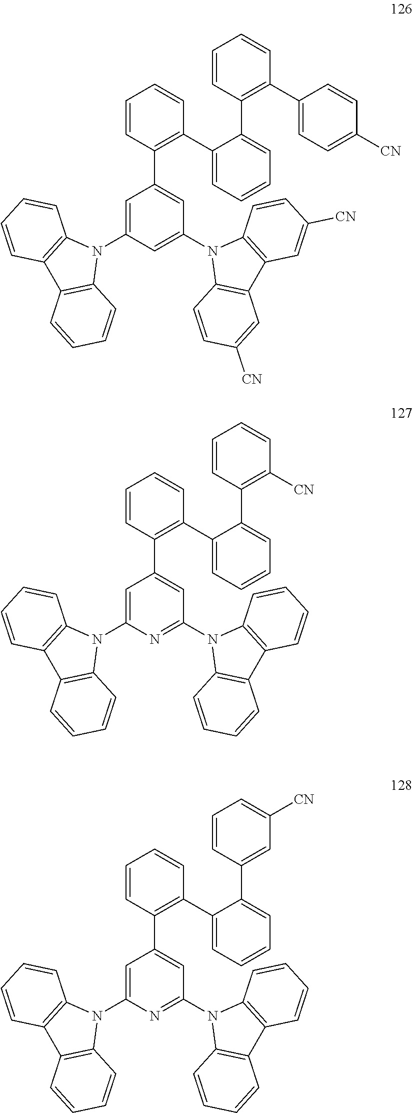

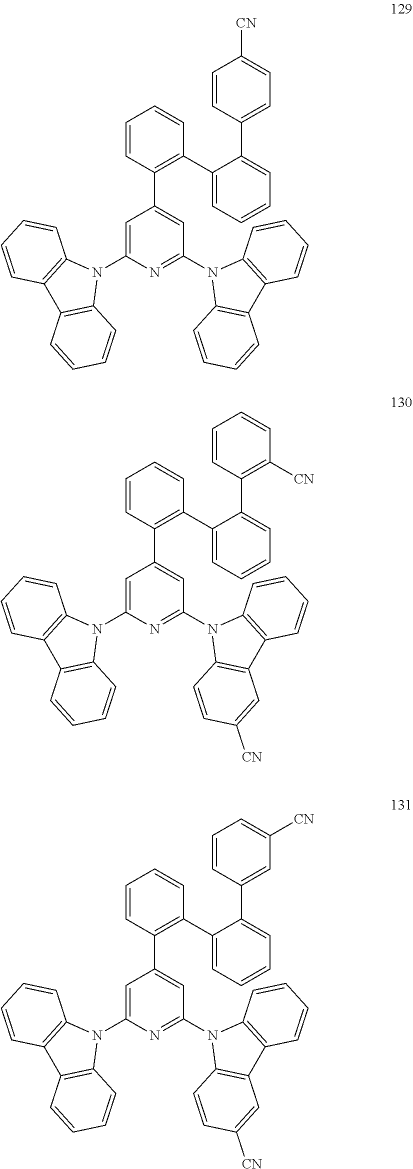

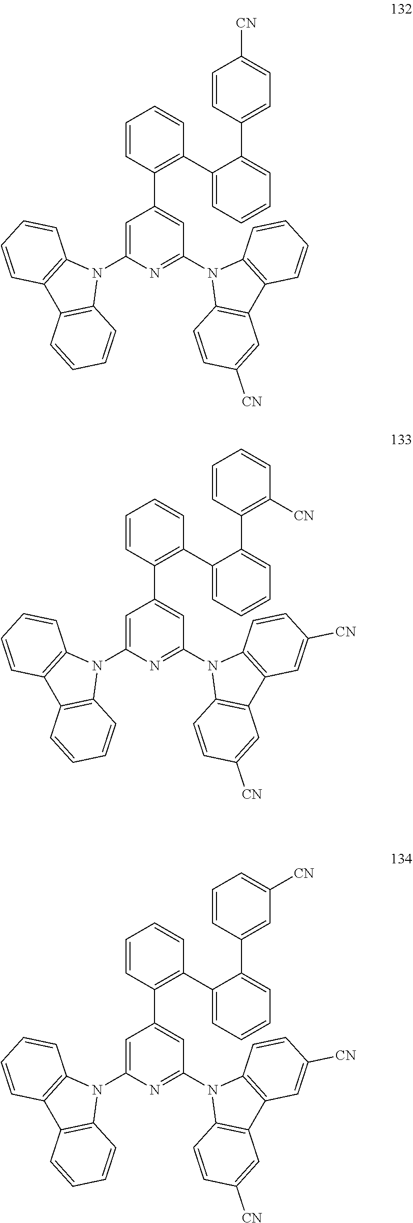 [SMILES: N#Cc1ccc(-c2ccccc2-c2ccccc2-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2-c2ccccc2-c2ccccc2C#N)cc(-n2c3ccccc3c3ccccc32)n1 ; N#Cc1cccc(-c2ccccc2-c2ccccc2-c2cc(-n3c4ccccc4c4ccccc43)nc(-n3c4ccc(C#N)cc4c4cc(C#N)ccc43)c2)c1]

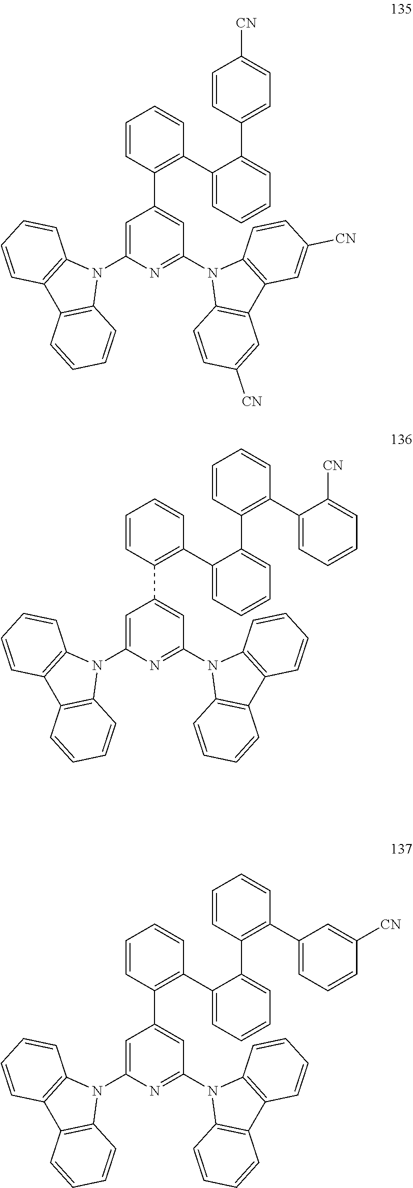

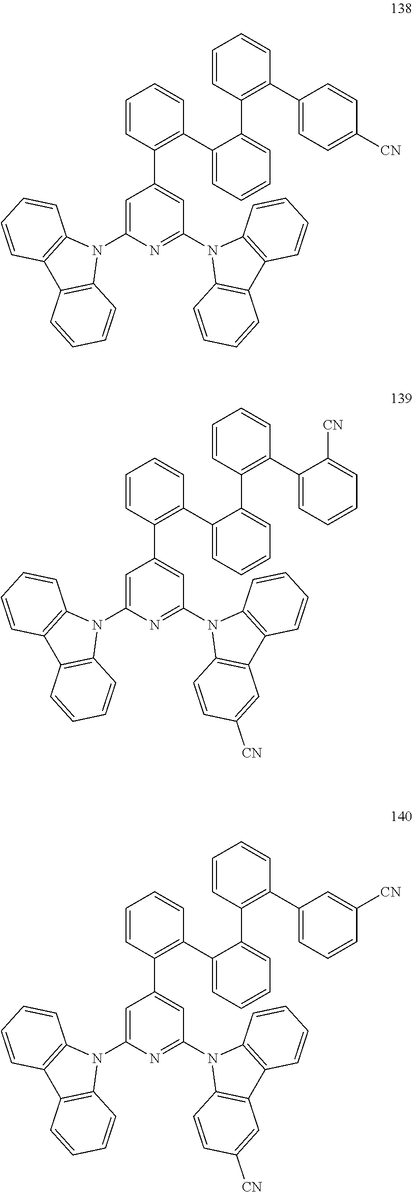 [SMILES: N#Cc1ccc(-c2ccccc2-c2ccccc2-c2ccccc2-c2cc(-n3c4ccccc4c4ccccc43)nc(-n3c4ccccc4c4ccccc43)c2)cc1 ; N#Cc1ccc2c(c1)c1ccccc1n2-c1cc(-c2ccccc2-c2ccccc2-c2ccccc2-c2ccccc2C#N)cc(-n2c3ccccc3c3ccccc32)n1 ; N#Cc1cccc(-c2ccccc2-c2ccccc2-c2ccccc2-c2cc(-n3c4ccccc4c4ccccc43)nc(-n3c4ccccc4c4cc(C#N)ccc43)c2)c1]

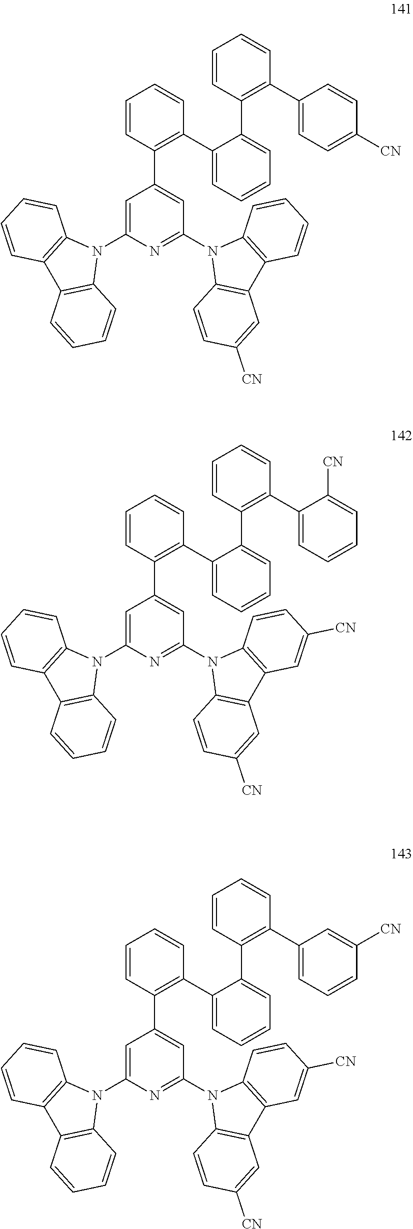 [SMILES: N#Cc1ccc(-c2ccccc2-c2ccccc2-c2ccccc2-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2-c2ccccc2-c2ccccc2-c2ccccc2C#N)cc(-n2c3ccccc3c3ccccc32)n1 ; N#Cc1cccc(-c2ccccc2-c2ccccc2-c2ccccc2-c2cc(-n3c4ccccc4c4ccccc43)nc(-n3c4ccc(C#N)cc4c4cc(C#N)ccc43)c2)c1]

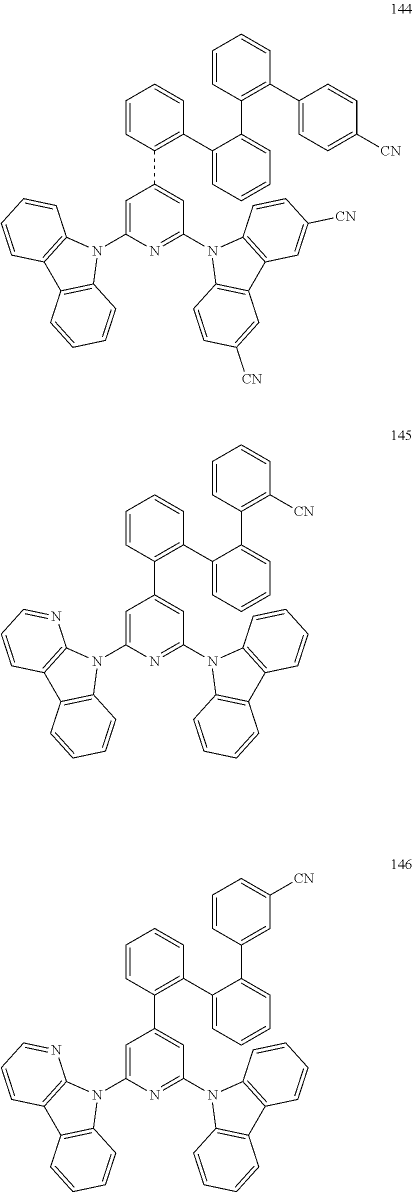

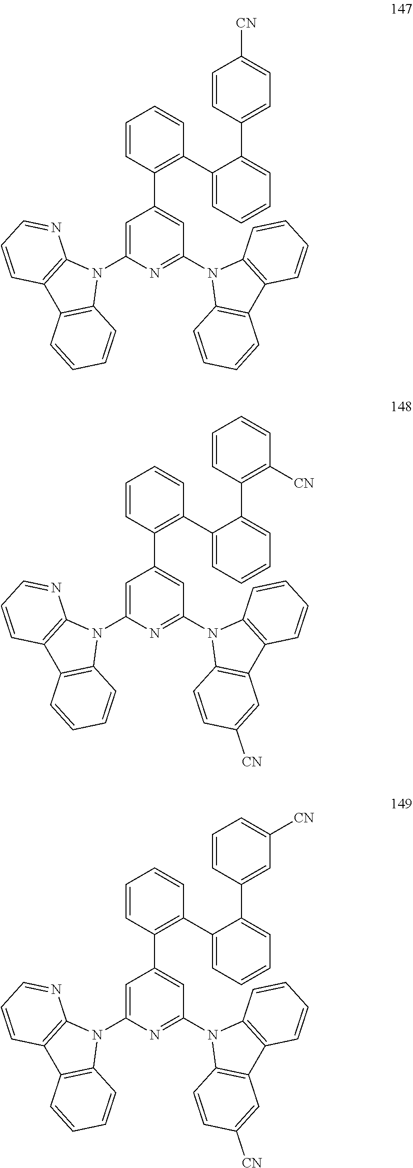 [SMILES: N#Cc1ccc(-c2ccccc2-c2ccccc2-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2-c2ccccc2-c2ccccc2C#N)cc(-n2c3ccccc3c3cccnc32)n1 ; N#Cc1cccc(-c2ccccc2-c2ccccc2-c2cc(-n3c4ccccc4c4cc(C#N)ccc43)nc(-n3c4ccccc4c4cccnc43)c2)c1]

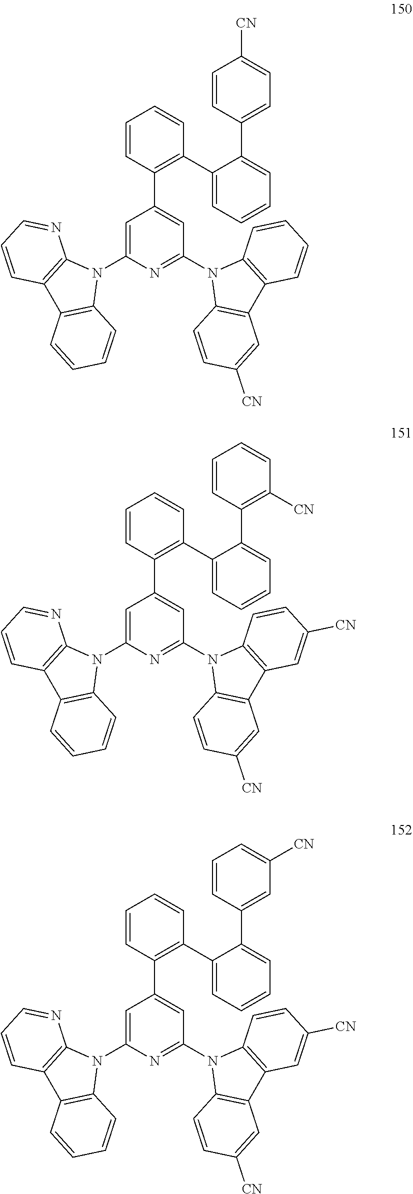

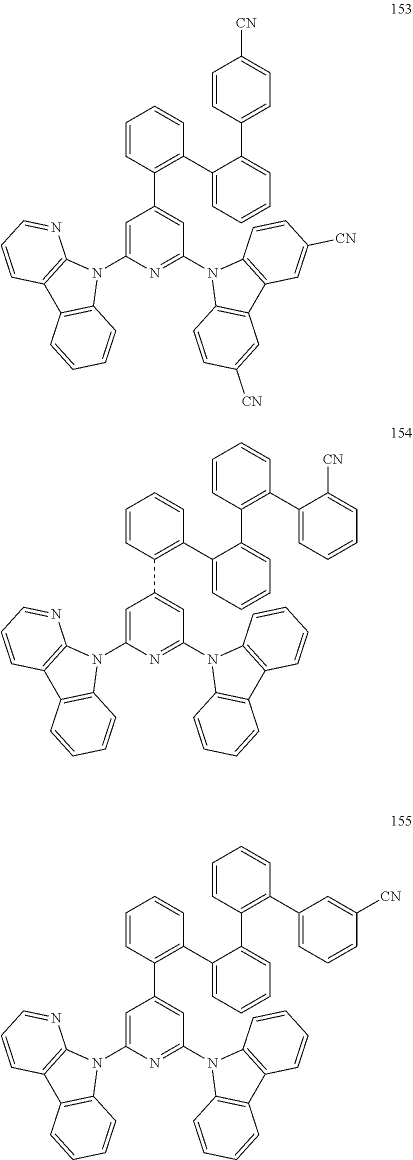

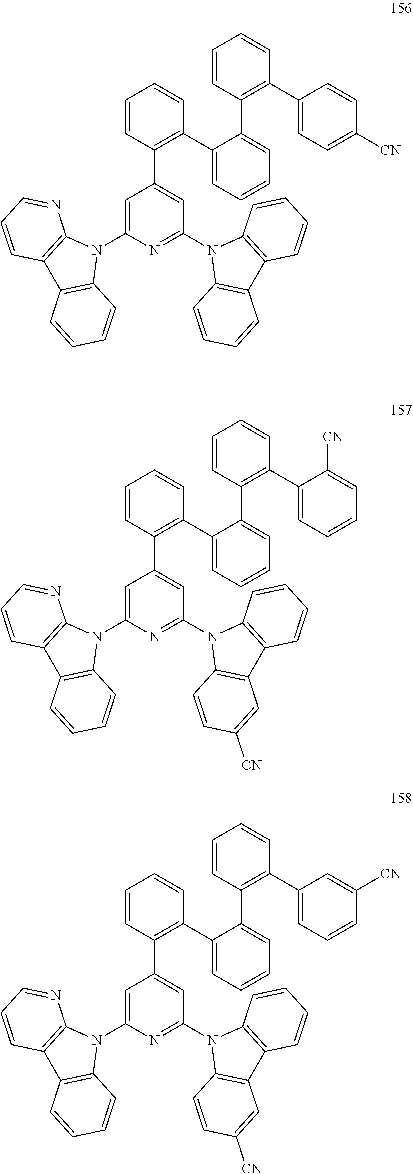 [SMILES: N#Cc1ccc(-c2ccccc2-c2ccccc2-c2ccccc2-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2-c2ccccc2-c2ccccc2-c2ccccc2C#N)cc(-n2c3ccccc3c3cccnc32)n1 ; N#Cc1cccc(-c2ccccc2-c2ccccc2-c2ccccc2-c2cc(-n3c4ccccc4c4cc(C#N)ccc43)nc(-n3c4ccccc4c4cccnc43)c2)c1]

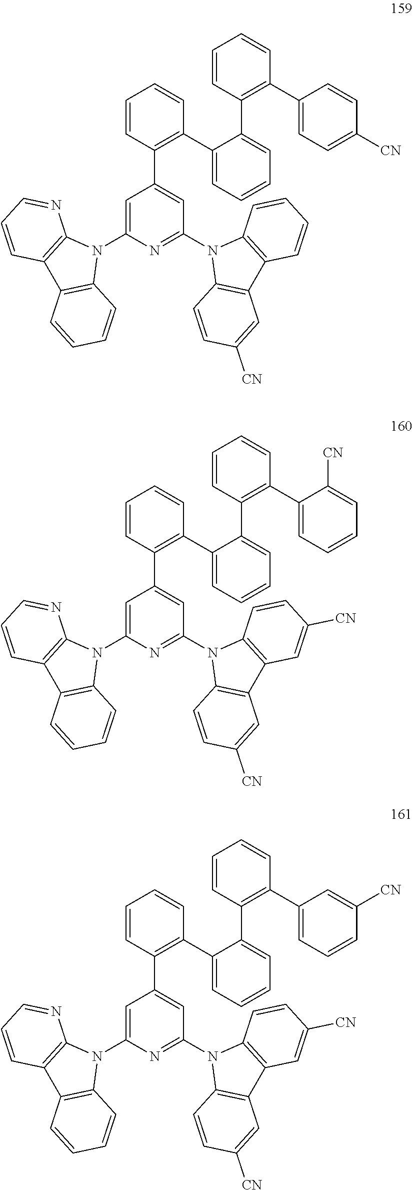 [SMILES: N#Cc1ccc(-c2ccccc2-c2ccccc2-c2ccccc2-c2cc(-n3c4ccccc4c4cc(C#N)ccc43)nc(-n3c4ccccc4c4cccnc43)c2)cc1 ; N#Cc1ccc2c(c1)c1cc(C#N)ccc1n2-c1cc(-c2ccccc2-c2ccccc2-c2ccccc2-c2ccccc2C#N)cc(-n2c3ccccc3c3cccnc32)n1 ; N#Cc1cccc(-c2ccccc2-c2ccccc2-c2ccccc2-c2cc(-n3c4ccc(C#N)cc4c4cc(C#N)ccc43)nc(-n3c4ccccc4c4cccnc43)c2)c1]

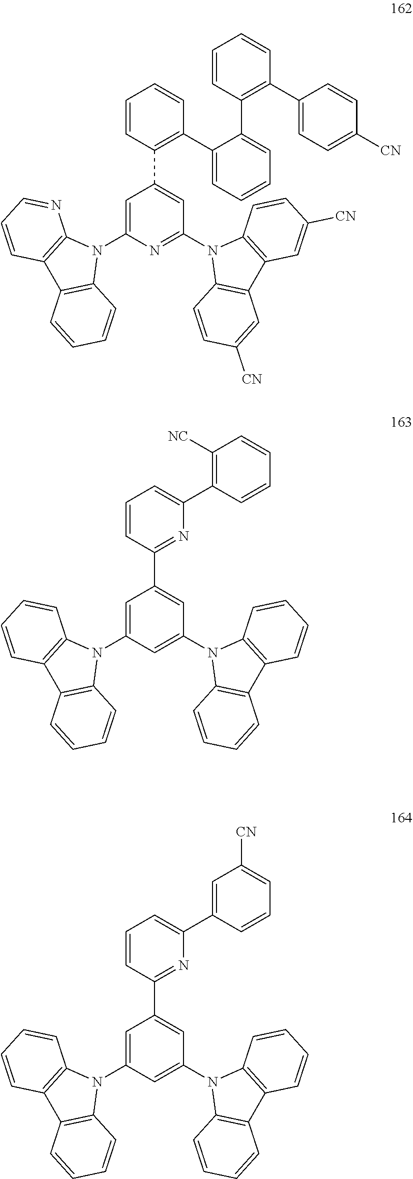

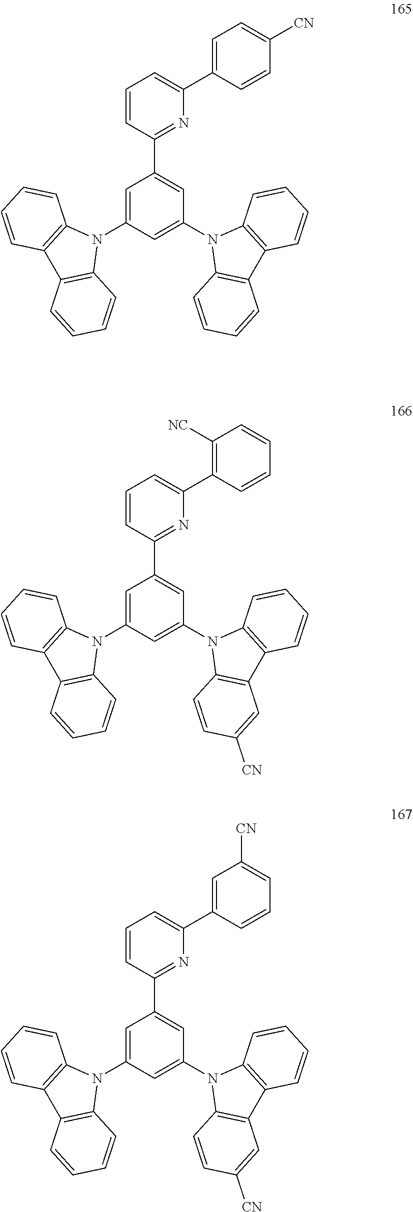 [SMILES: N#Cc1ccc(-c2cccc(-c3cc(-n4c5ccccc5c5ccccc54)cc(-n4c5ccccc5c5ccccc54)c3)n2)cc1 ; N#Cc1ccc2c(c1)c1ccccc1n2-c1cc(-c2cccc(-c3ccccc3C#N)n2)cc(-n2c3ccccc3c3ccccc32)c1 ; N#Cc1cccc(-c2cccc(-c3cc(-n4c5ccccc5c5ccccc54)cc(-n4c5ccccc5c5cc(C#N)ccc54)c3)n2)c1]

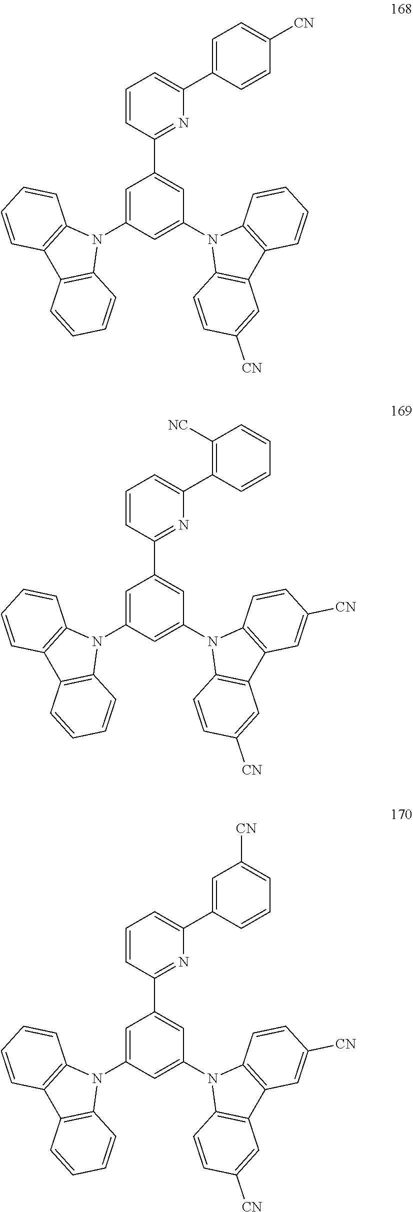 [SMILES: N#Cc1ccc(-c2cccc(-c3cc(-n4c5ccccc5c5ccccc54)cc(-n4c5ccccc5c5cc(C#N)ccc54)c3)n2)cc1 ; N#Cc1ccc2c(c1)c1cc(C#N)ccc1n2-c1cc(-c2cccc(-c3ccccc3C#N)n2)cc(-n2c3ccccc3c3ccccc32)c1 ; N#Cc1cccc(-c2cccc(-c3cc(-n4c5ccccc5c5ccccc54)cc(-n4c5ccc(C#N)cc5c5cc(C#N)ccc54)c3)n2)c1]

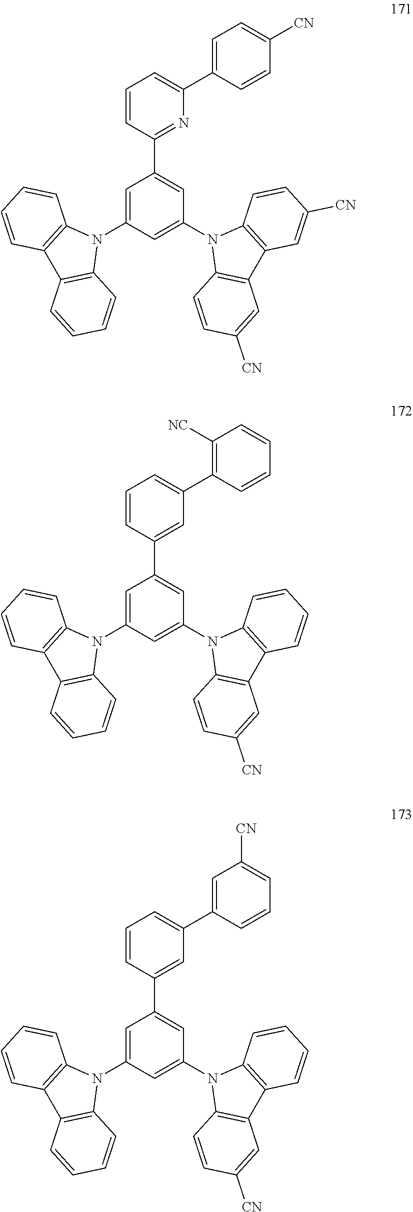 [SMILES: N#Cc1ccc(-c2cccc(-c3cc(-n4c5ccccc5c5ccccc54)cc(-n4c5ccc(C#N)cc5c5cc(C#N)ccc54)c3)n2)cc1 ; N#Cc1ccc2c(c1)c1ccccc1n2-c1cc(-c2cccc(-c3ccccc3C#N)c2)cc(-n2c3ccccc3c3ccccc32)c1 ; N#Cc1cccc(-c2cccc(-c3cc(-n4c5ccccc5c5ccccc54)cc(-n4c5ccccc5c5cc(C#N)ccc54)c3)c2)c1]

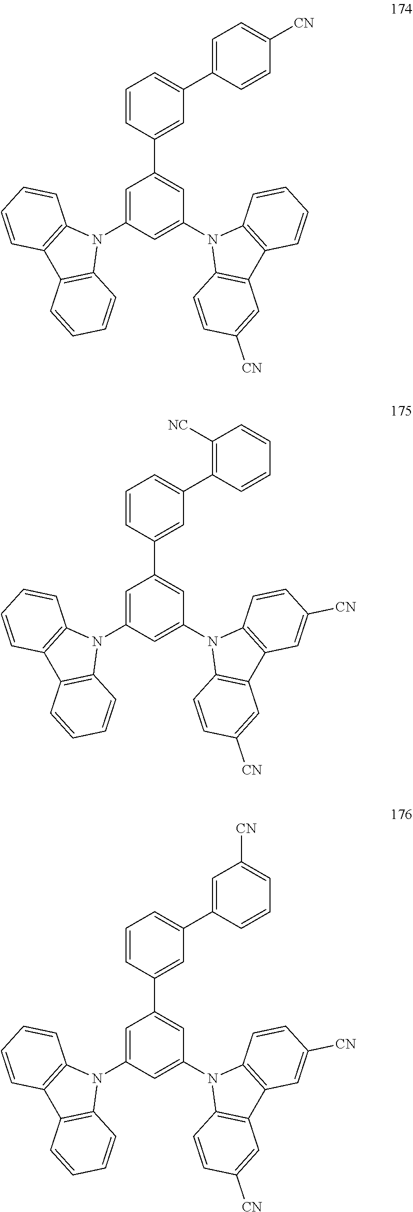 [SMILES: N#Cc1ccc(-c2cccc(-c3cc(-n4c5ccccc5c5ccccc54)cc(-n4c5ccccc5c5cc(C#N)ccc54)c3)c2)cc1 ; N#Cc1ccc2c(c1)c1cc(C#N)ccc1n2-c1cc(-c2cccc(-c3ccccc3C#N)c2)cc(-n2c3ccccc3c3ccccc32)c1 ; N#Cc1cccc(-c2cccc(-c3cc(-n4c5ccccc5c5ccccc54)cc(-n4c5ccc(C#N)cc5c5cc(C#N)ccc54)c3)c2)c1]

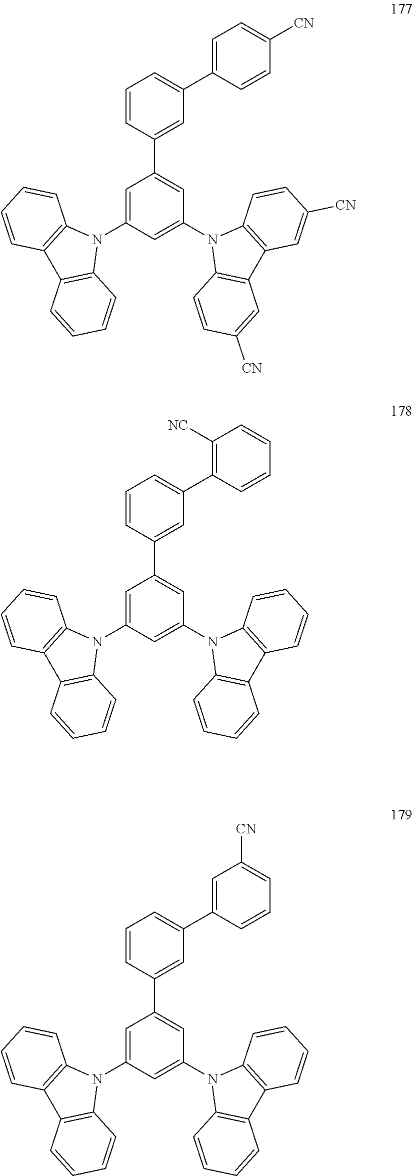 [SMILES: N#Cc1ccc(-c2cccc(-c3cc(-n4c5ccccc5c5ccccc54)cc(-n4c5ccc(C#N)cc5c5cc(C#N)ccc54)c3)c2)cc1 ; N#Cc1ccccc1-c1cccc(-c2cc(-n3c4ccccc4c4ccccc43)cc(-n3c4ccccc4c4ccccc43)c2)c1 ; N#Cc1cccc(-c2cccc(-c3cc(-n4c5ccccc5c5ccccc54)cc(-n4c5ccccc5c5ccccc54)c3)c2)c1]

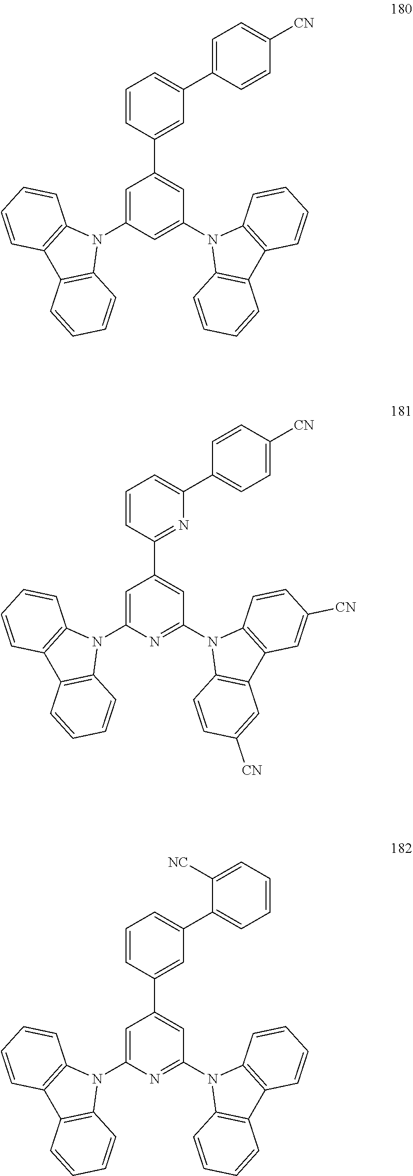 [SMILES: N#Cc1ccc(-c2cccc(-c3cc(-n4c5ccccc5c5ccccc54)cc(-n4c5ccccc5c5ccccc54)c3)c2)cc1 ; N#Cc1ccc(-c2cccc(-c3cc(-n4c5ccccc5c5ccccc54)nc(-n4c5ccc(C#N)cc5c5cc(C#N)ccc54)c3)n2)cc1 ; N#Cc1ccccc1-c1cccc(-c2cc(-n3c4ccccc4c4ccccc43)nc(-n3c4ccccc4c4ccccc43)c2)c1]

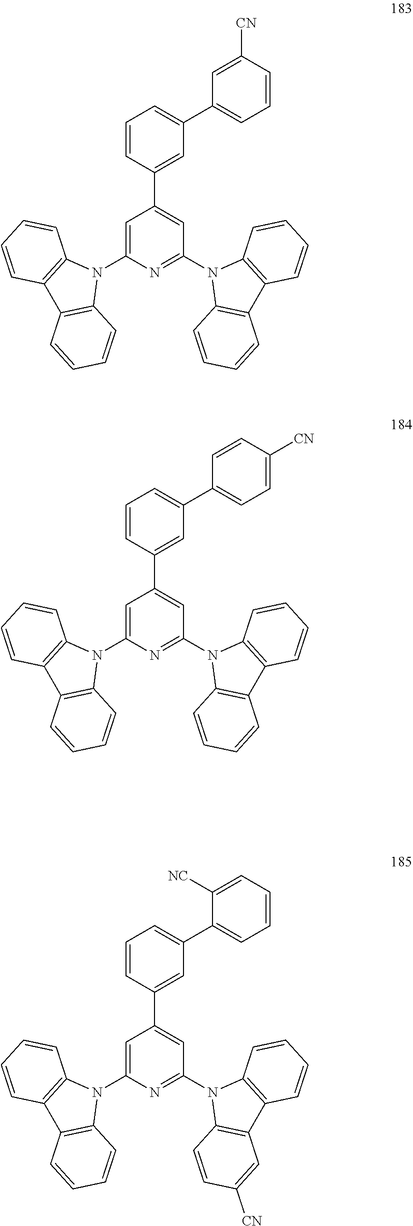 [SMILES: N#Cc1cccc(-c2cccc(-c3cc(-n4c5ccccc5c5ccccc54)nc(-n4c5ccccc5c5ccccc54)c3)c2)c1 ; N#Cc1ccc(-c2cccc(-c3cc(-n4c5ccccc5c5ccccc54)nc(-n4c5ccccc5c5ccccc54)c3)c2)cc1 ; N#Cc1ccc2c(c1)c1ccccc1n2-c1cc(-c2cccc(-c3ccccc3C#N)c2)cc(-n2c3ccccc3c3ccccc32)n1]

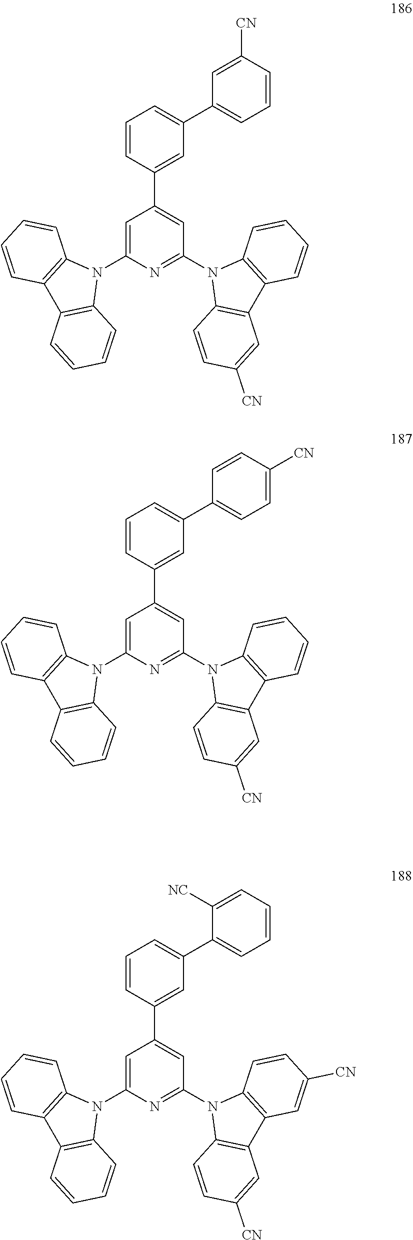 [SMILES: N#Cc1cccc(-c2cccc(-c3cc(-n4c5ccccc5c5ccccc54)nc(-n4c5ccccc5c5cc(C#N)ccc54)c3)c2)c1 ; N#Cc1ccc(-c2cccc(-c3cc(-n4c5ccccc5c5ccccc54)nc(-n4c5ccccc5c5cc(C#N)ccc54)c3)c2)cc1 ; N#Cc1ccc2c(c1)c1cc(C#N)ccc1n2-c1cc(-c2cccc(-c3ccccc3C#N)c2)cc(-n2c3ccccc3c3ccccc32)n1]

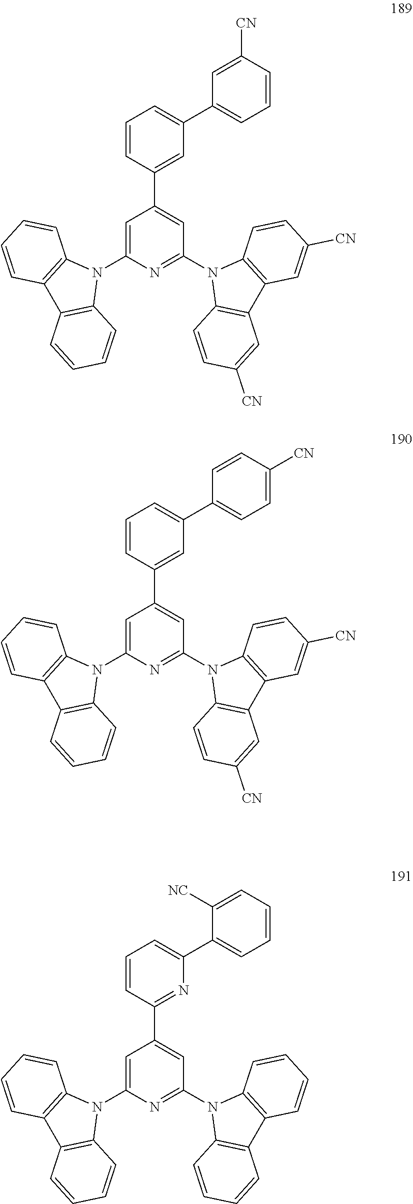 [SMILES: N#Cc1cccc(-c2cccc(-c3cc(-n4c5ccccc5c5ccccc54)nc(-n4c5ccc(C#N)cc5c5cc(C#N)ccc54)c3)c2)c1 ; N#Cc1ccc(-c2cccc(-c3cc(-n4c5ccccc5c5ccccc54)nc(-n4c5ccc(C#N)cc5c5cc(C#N)ccc54)c3)c2)cc1 ; N#Cc1ccccc1-c1cccc(-c2cc(-n3c4ccccc4c4ccccc43)nc(-n3c4ccccc4c4ccccc43)c2)n1]

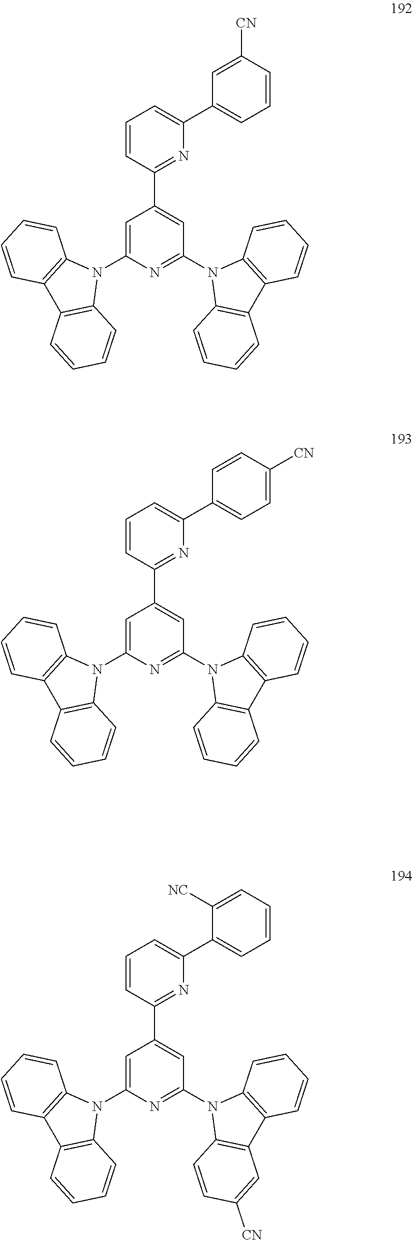 [SMILES: N#Cc1cccc(-c2cccc(-c3cc(-n4c5ccccc5c5ccccc54)nc(-n4c5ccccc5c5ccccc54)c3)n2)c1 ; N#Cc1ccc(-c2cccc(-c3cc(-n4c5ccccc5c5ccccc54)nc(-n4c5ccccc5c5ccccc54)c3)n2)cc1 ; N#Cc1ccc2c(c1)c1ccccc1n2-c1cc(-c2cccc(-c3ccccc3C#N)n2)cc(-n2c3ccccc3c3ccccc32)n1]

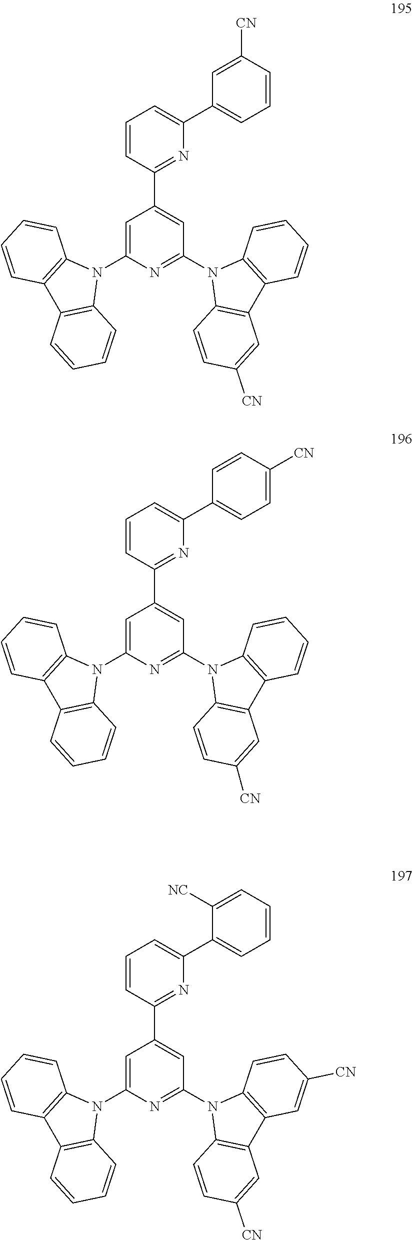 [SMILES: N#Cc1cccc(-c2cccc(-c3cc(-n4c5ccccc5c5ccccc54)nc(-n4c5ccccc5c5cc(C#N)ccc54)c3)n2)c1 ; N#Cc1ccc(-c2cccc(-c3cc(-n4c5ccccc5c5ccccc54)nc(-n4c5ccccc5c5cc(C#N)ccc54)c3)n2)cc1 ; N#Cc1ccc2c(c1)c1cc(C#N)ccc1n2-c1cc(-c2cccc(-c3ccccc3C#N)n2)cc(-n2c3ccccc3c3ccccc32)n1]

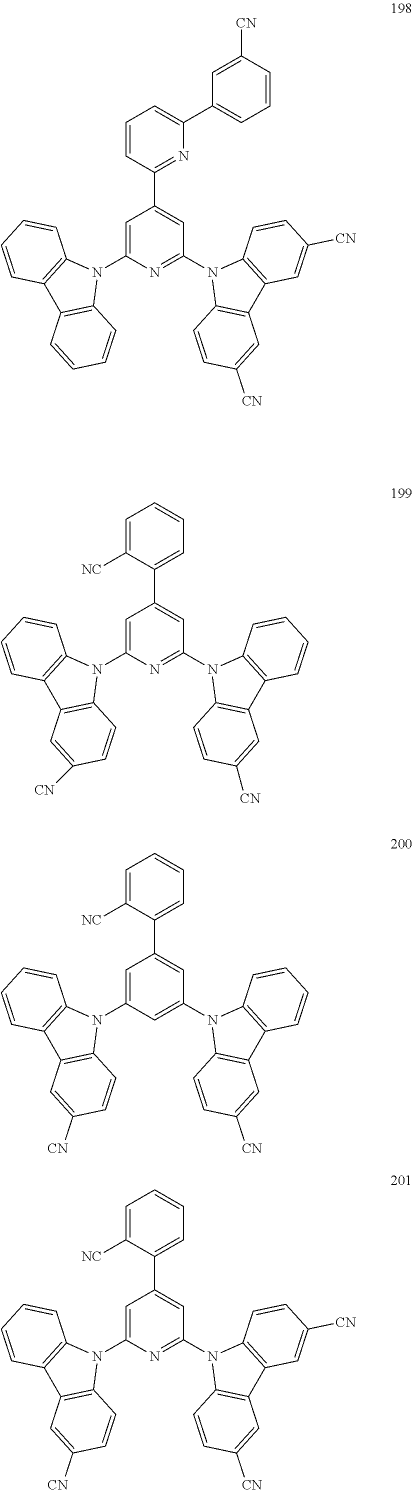

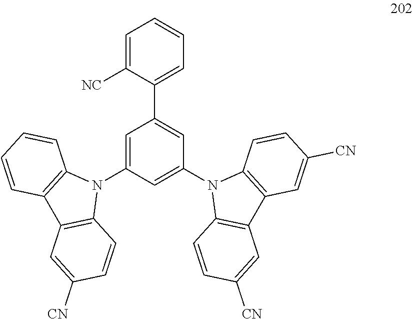

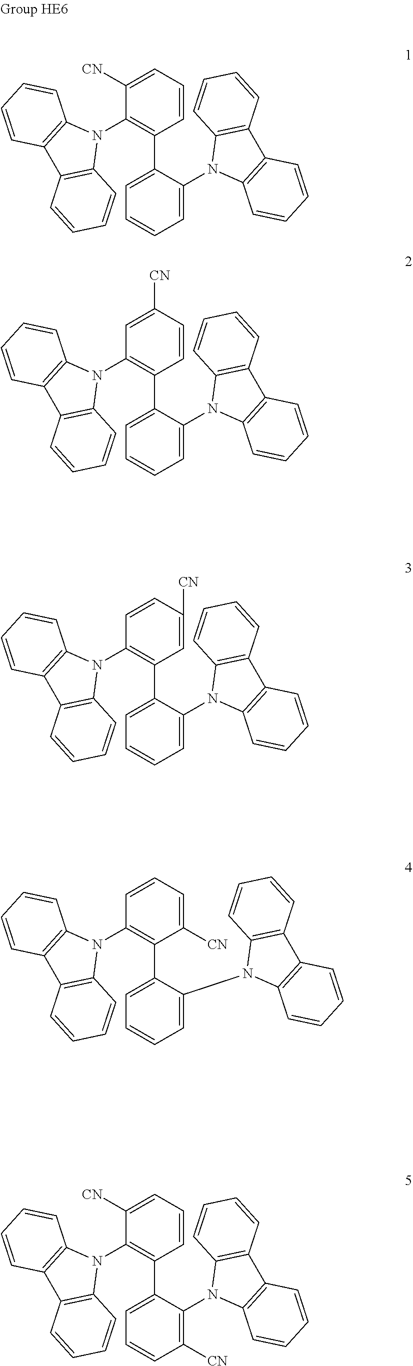

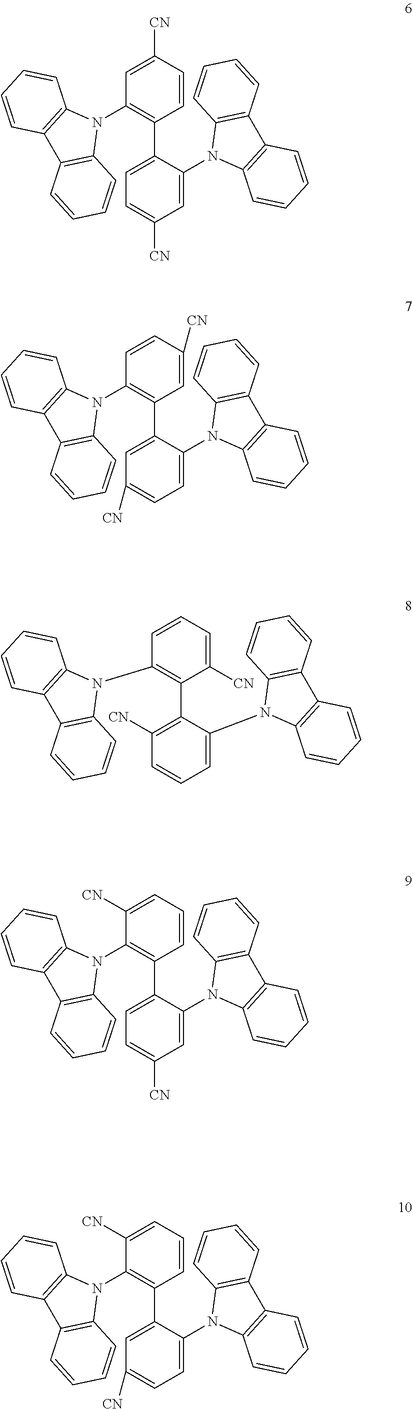

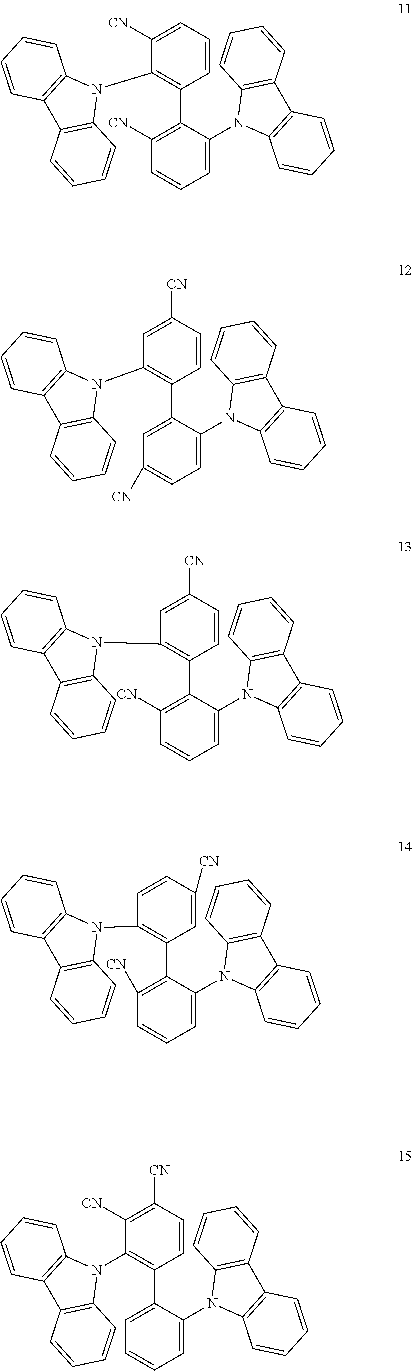

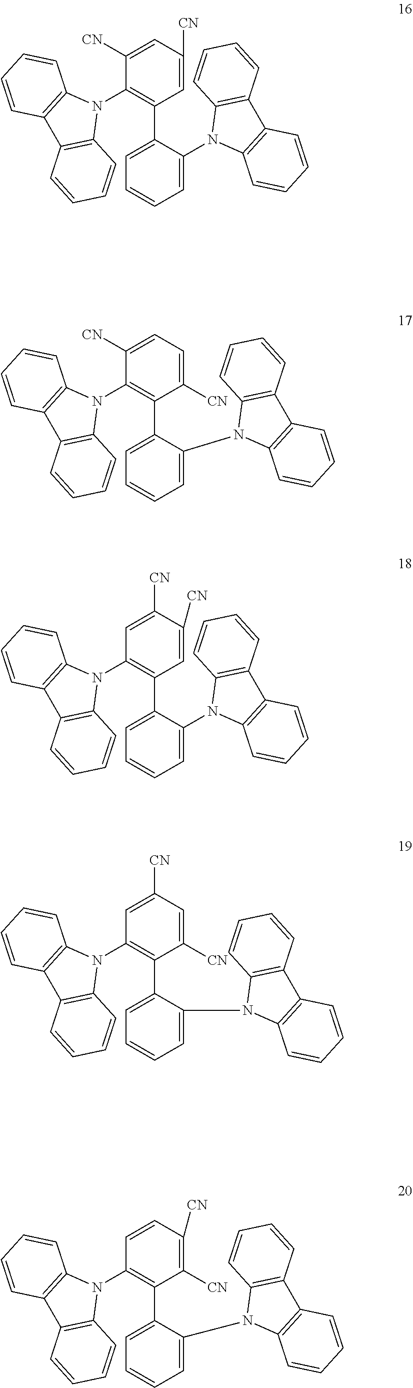

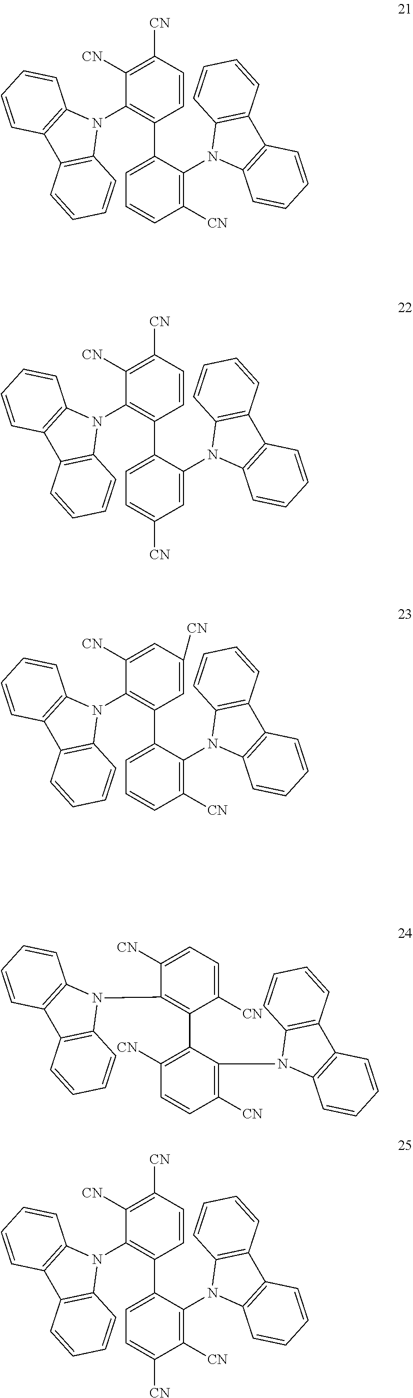

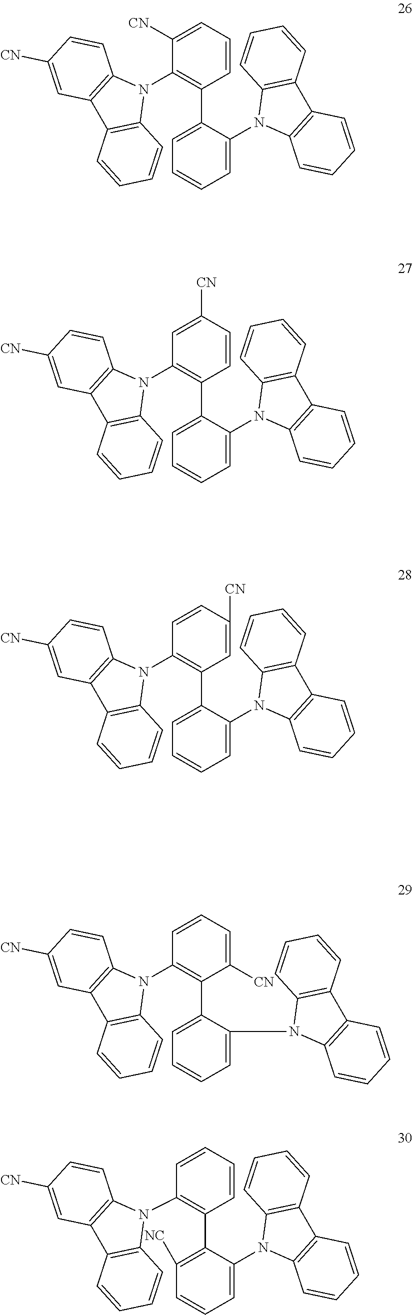

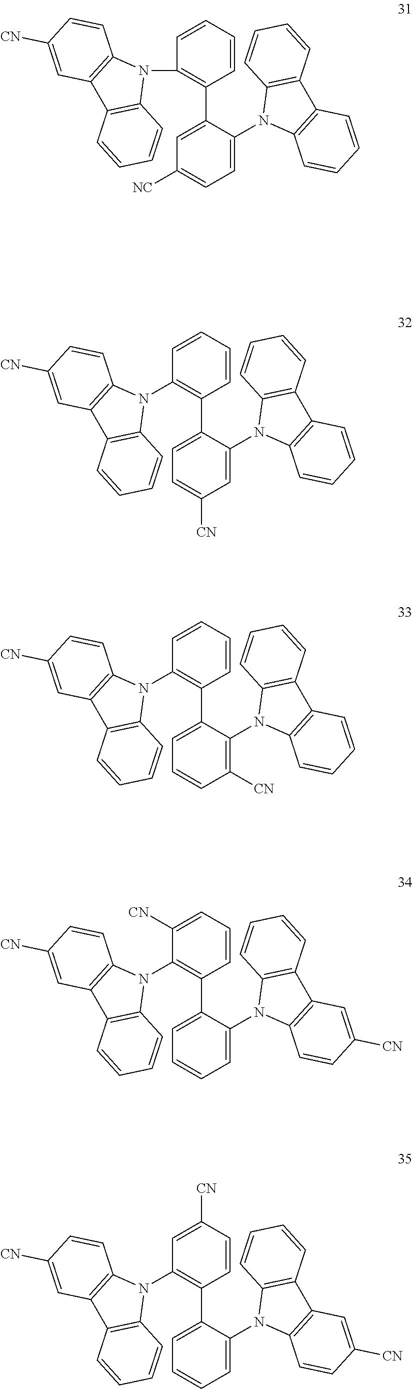

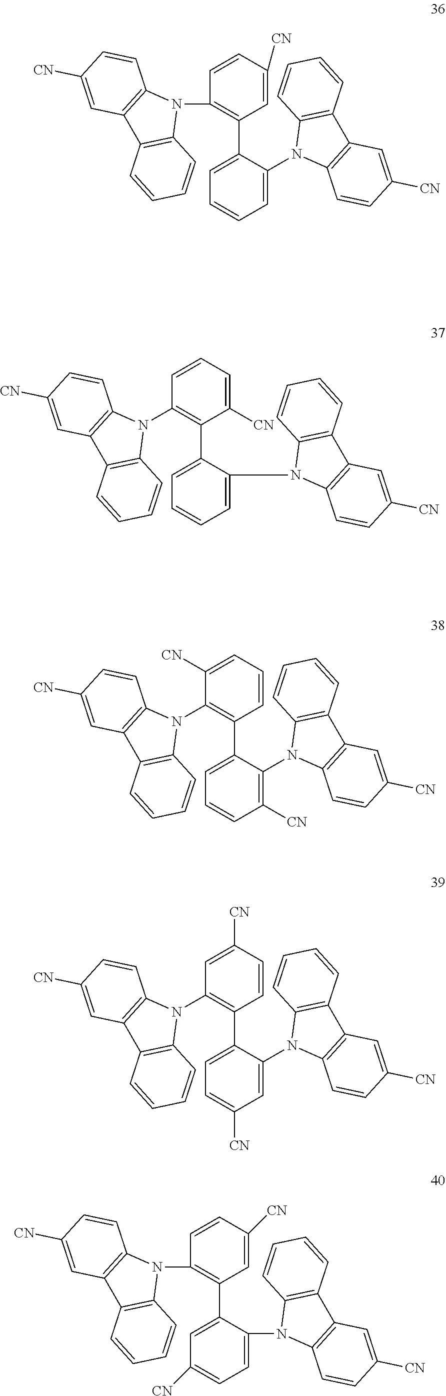

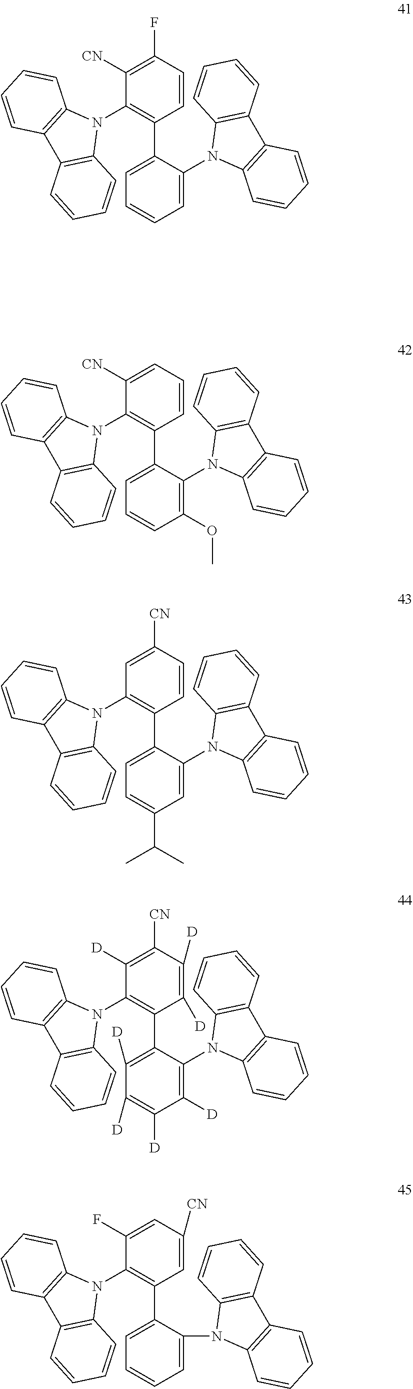

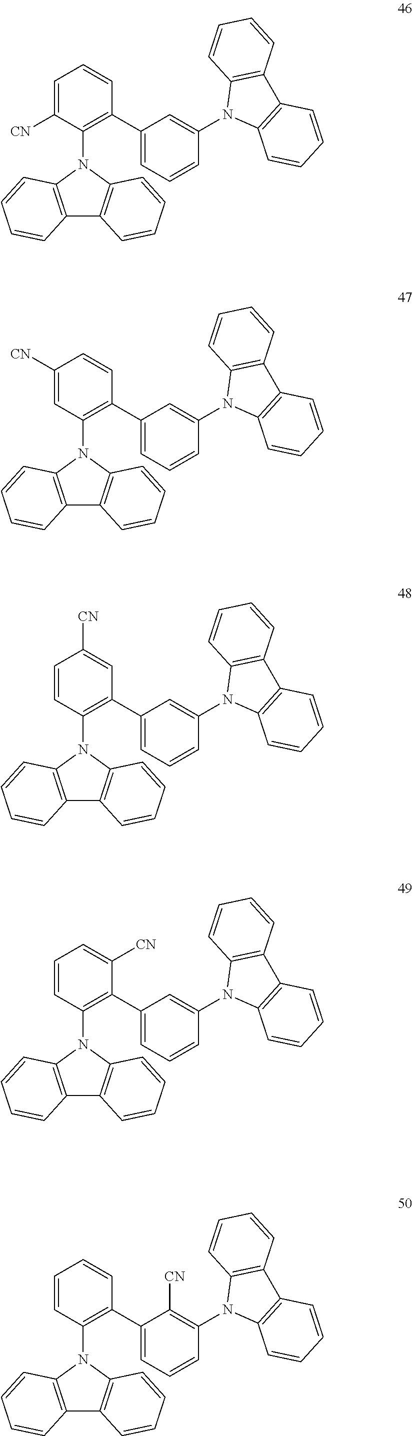

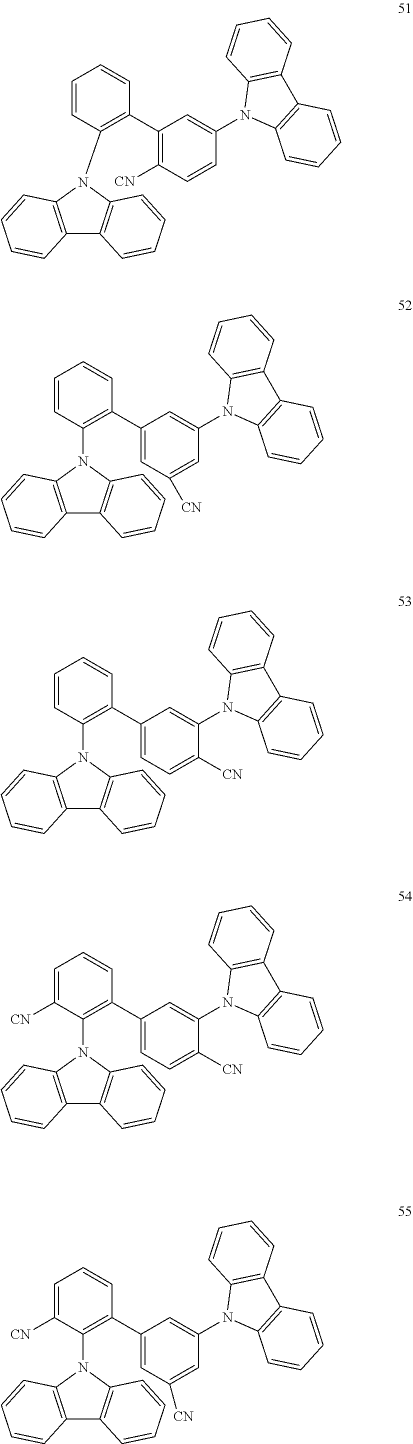

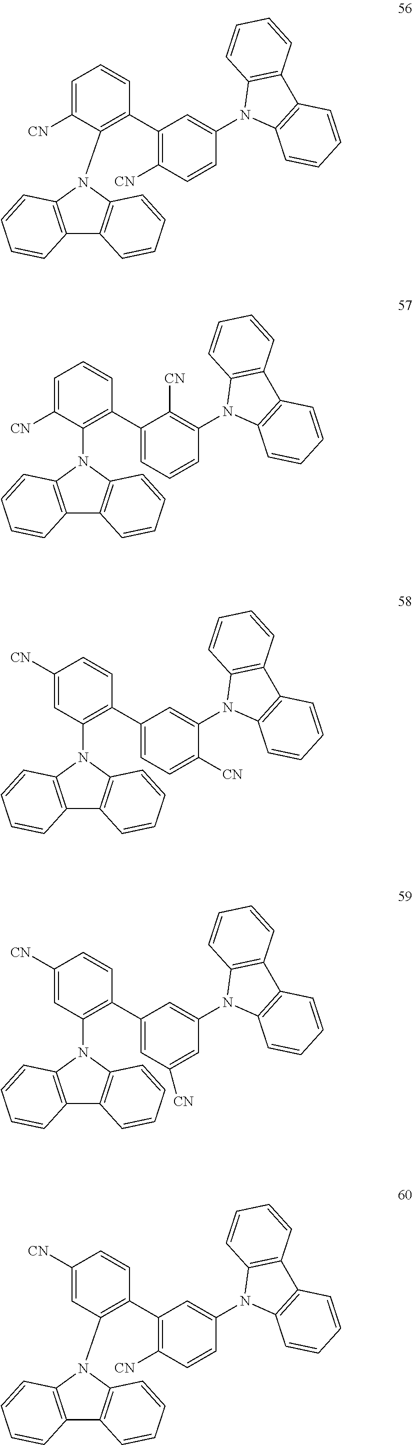

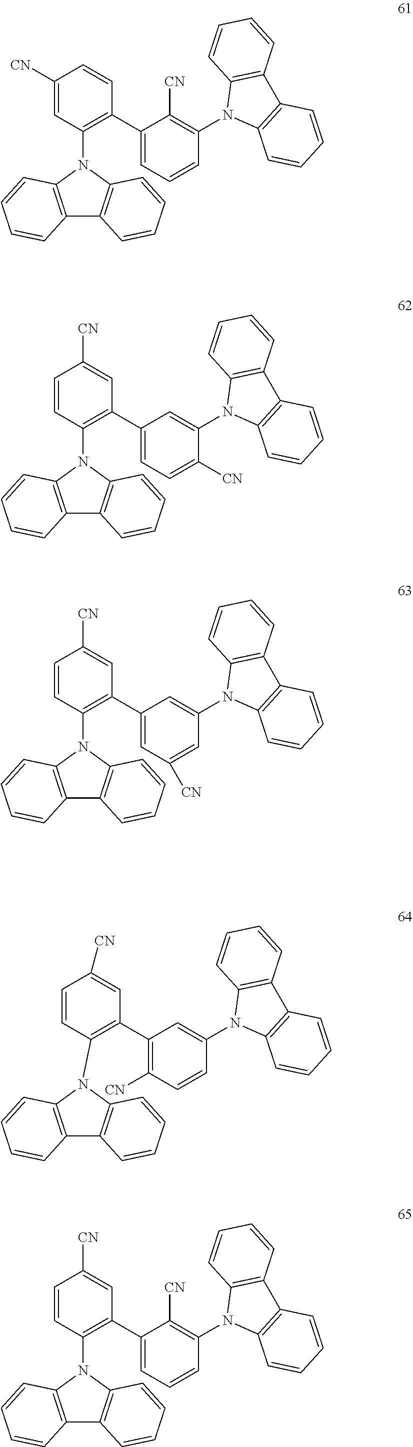

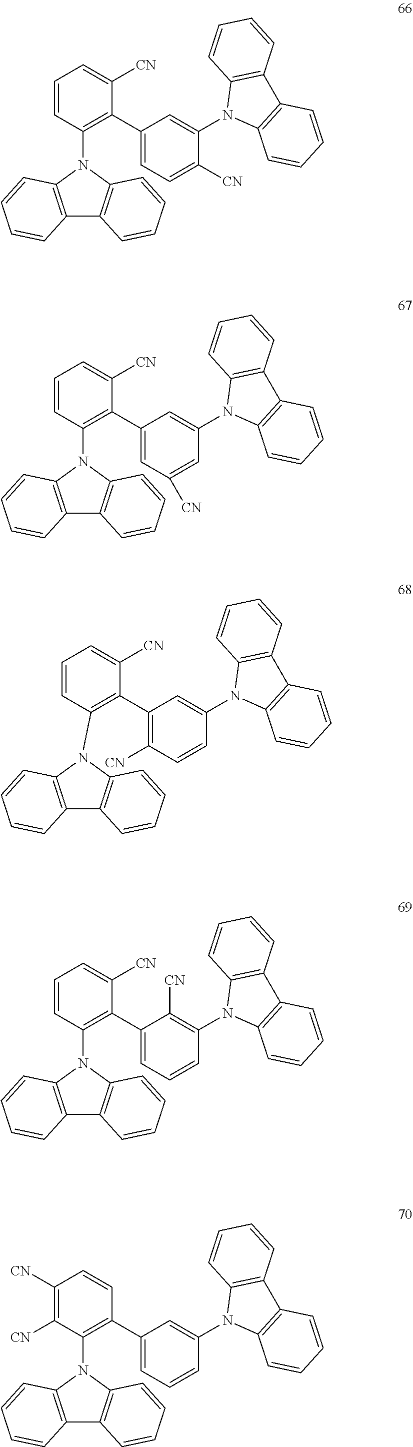

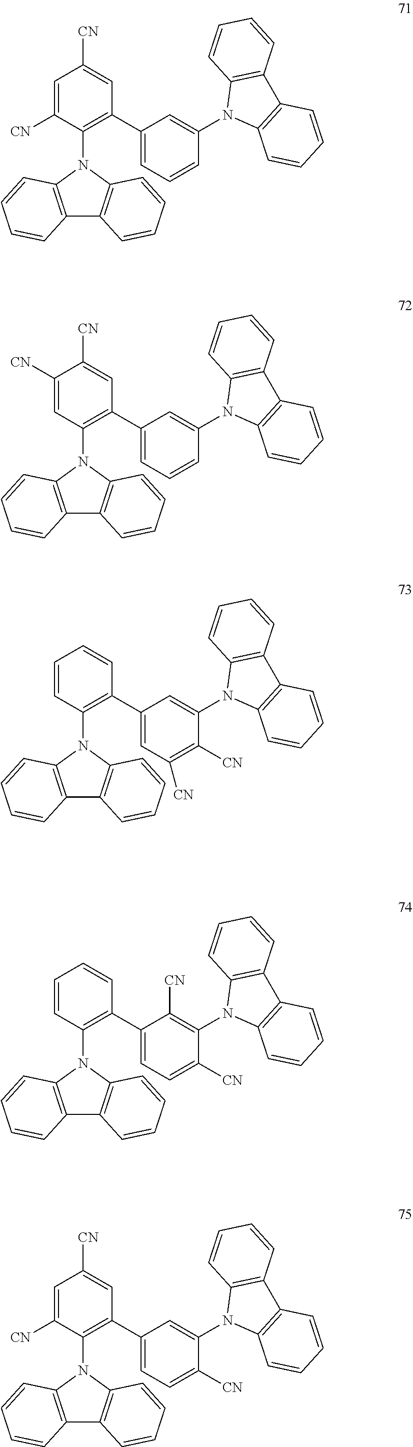

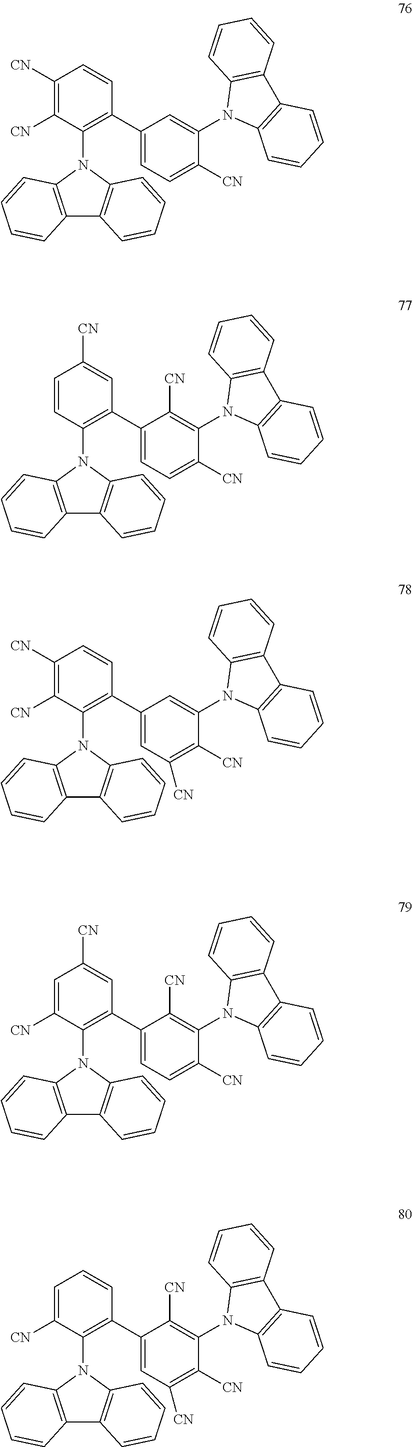

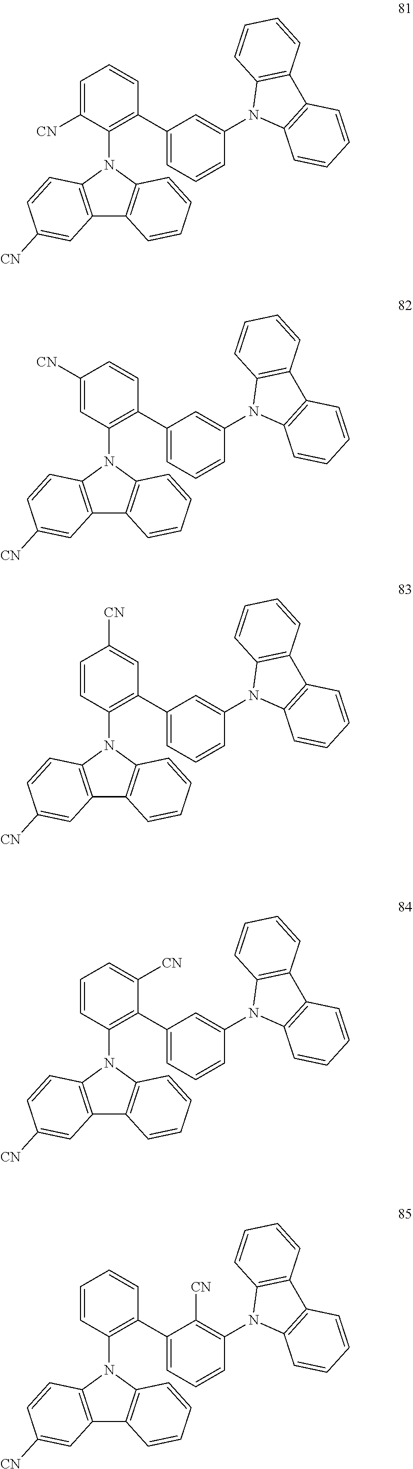

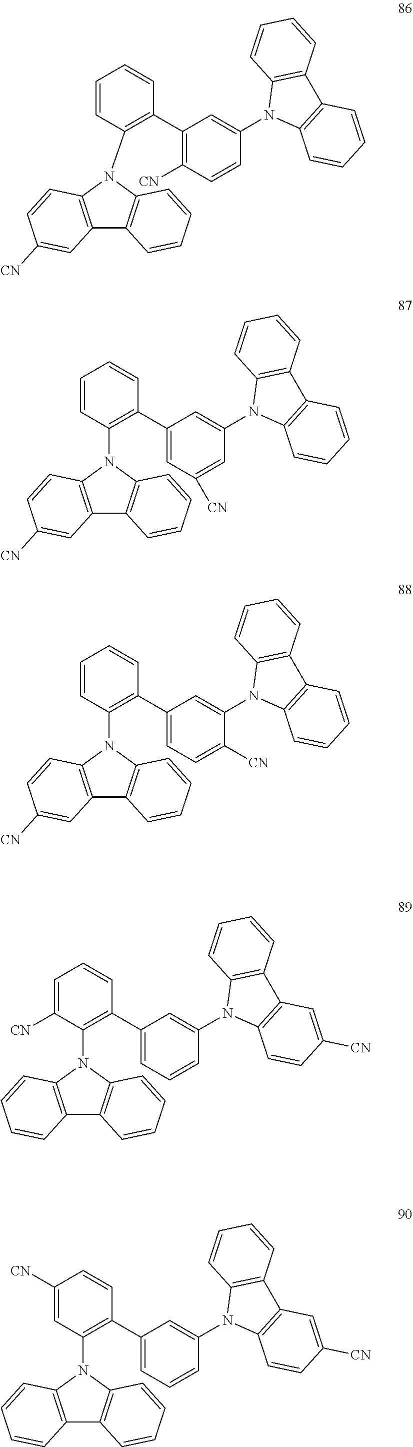

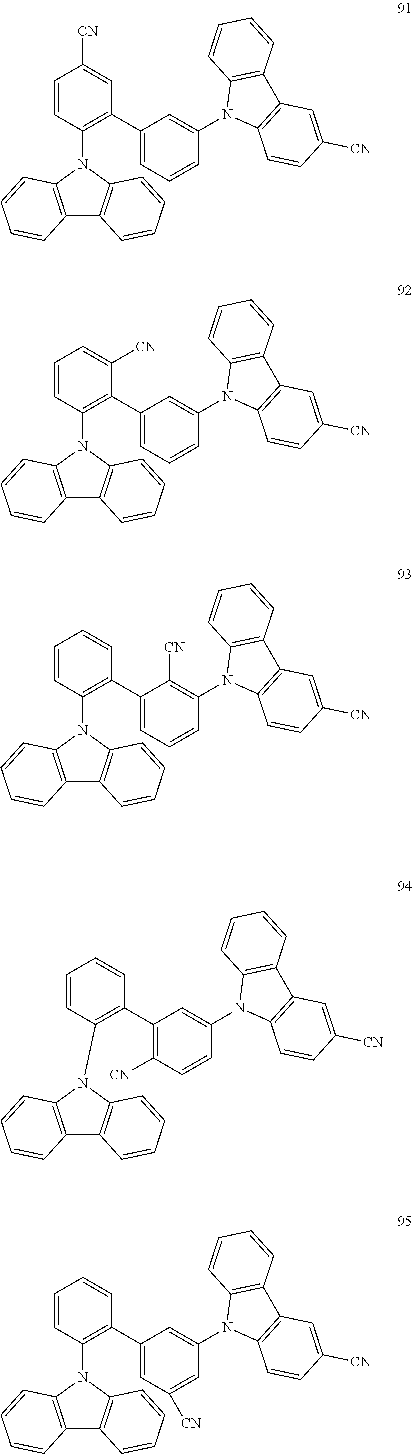

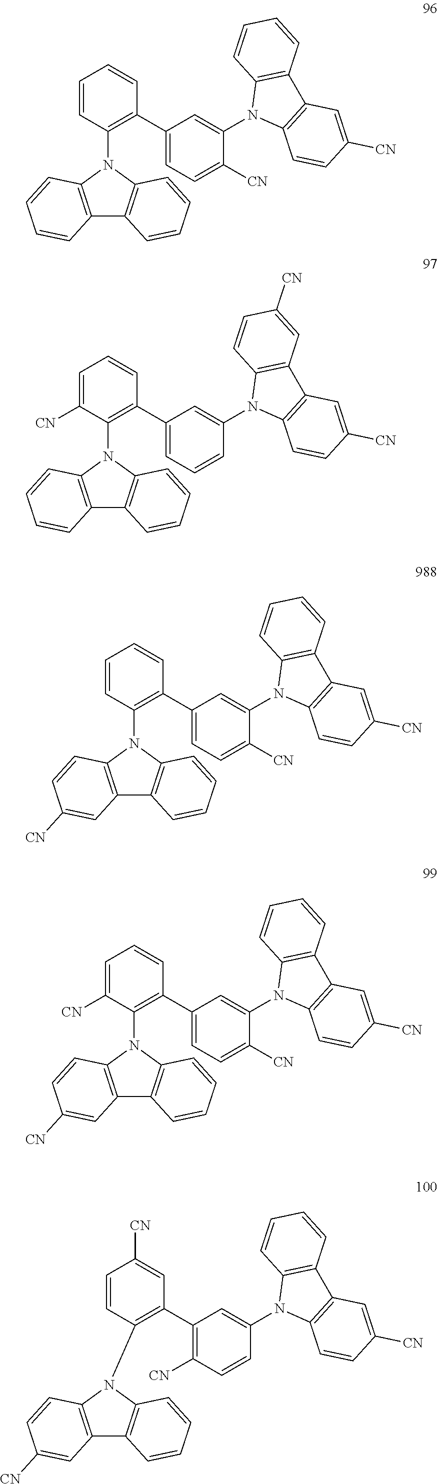

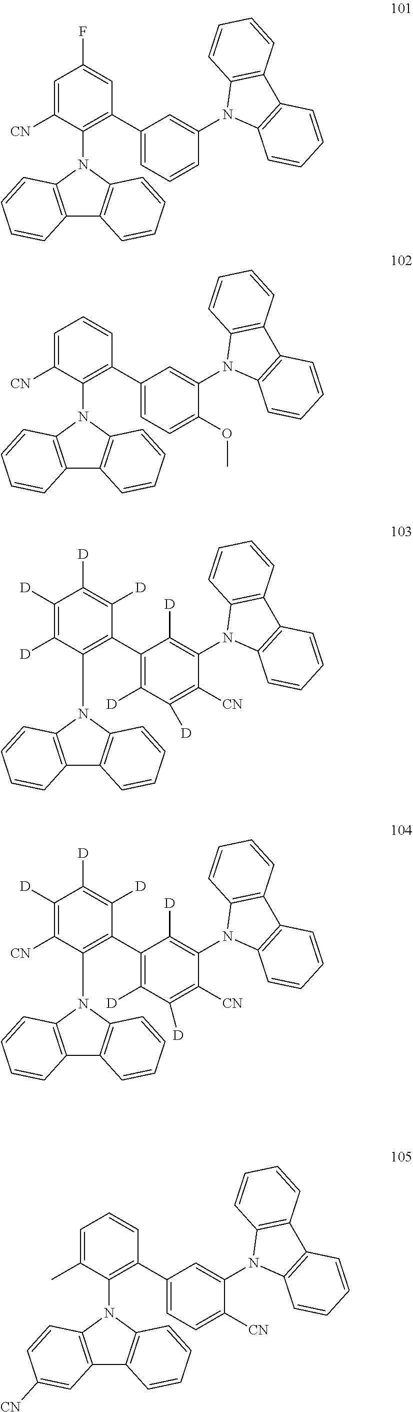

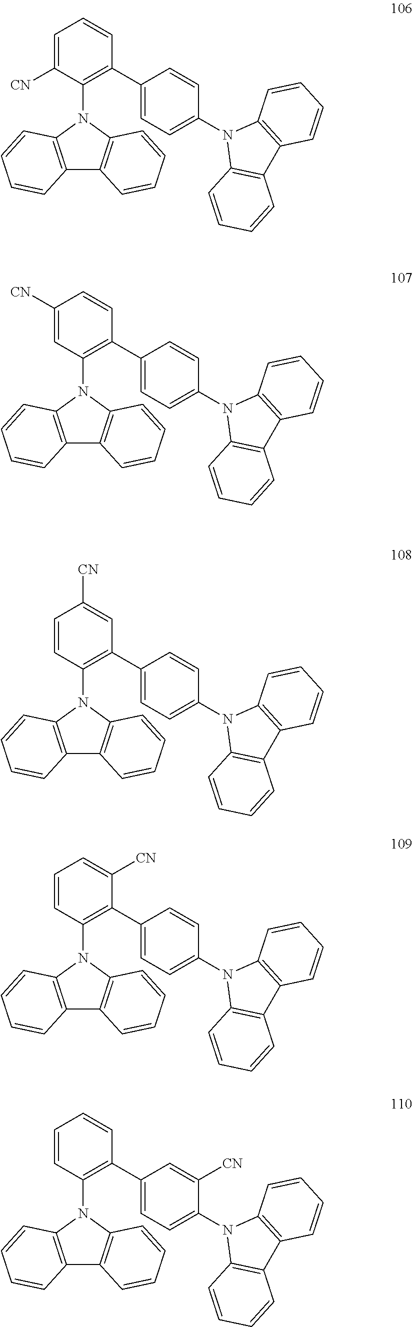

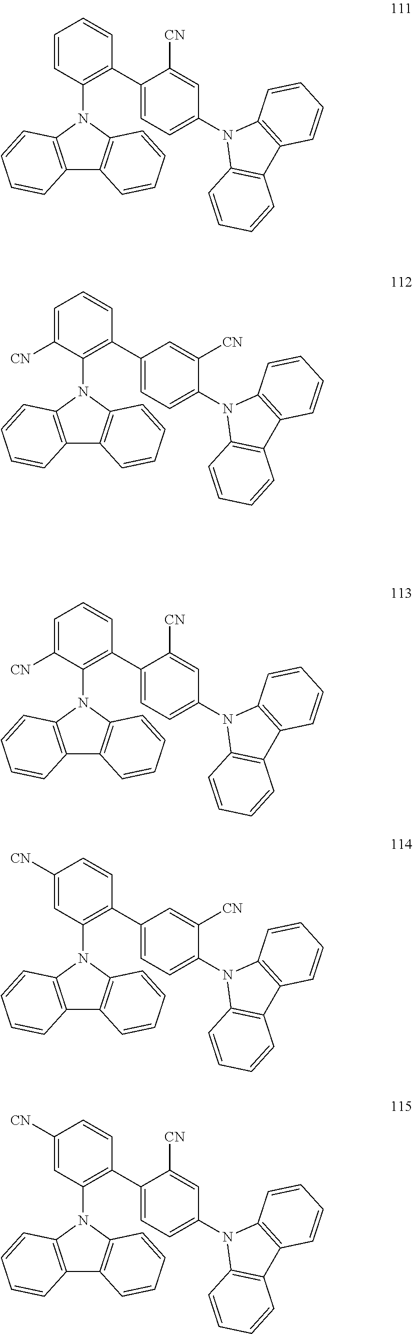

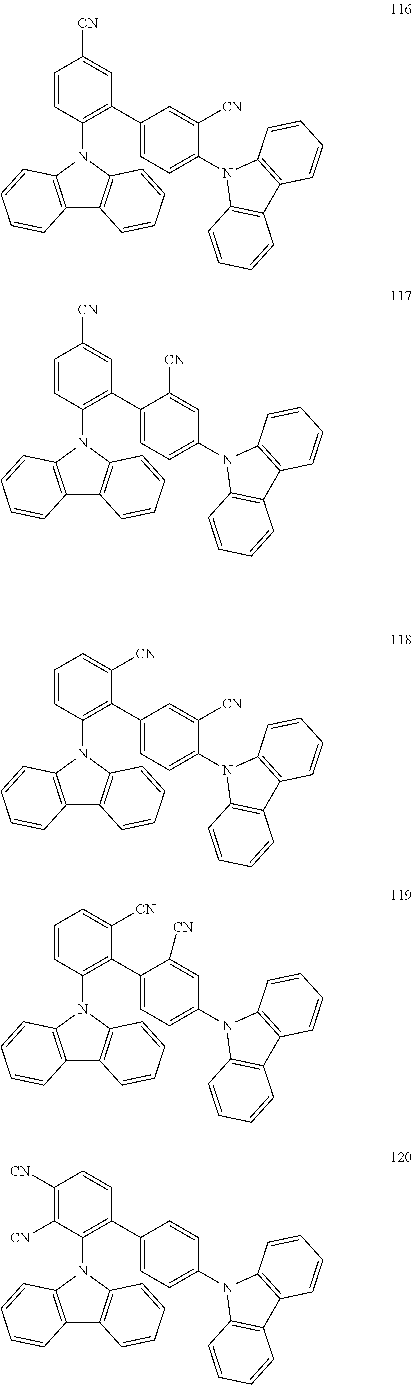

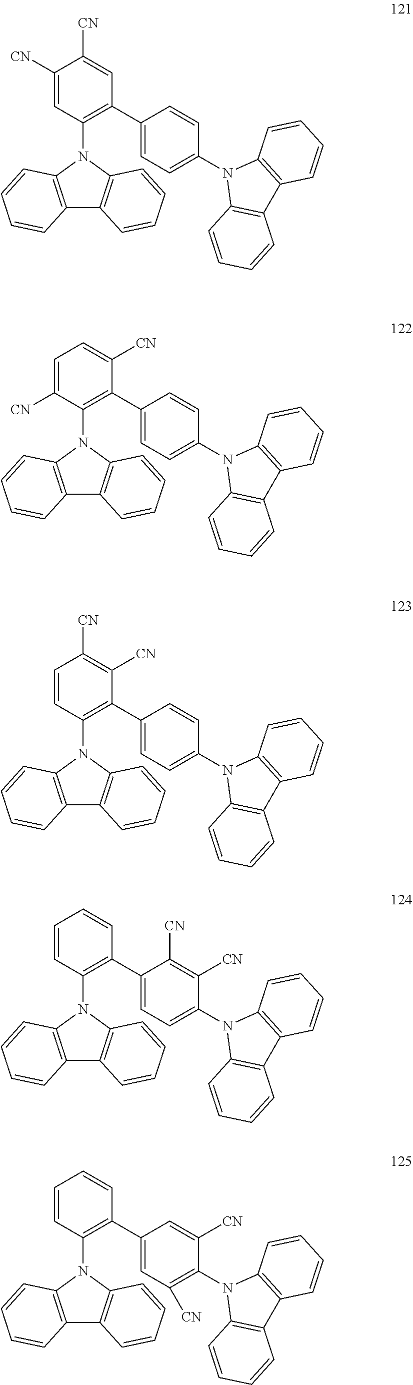

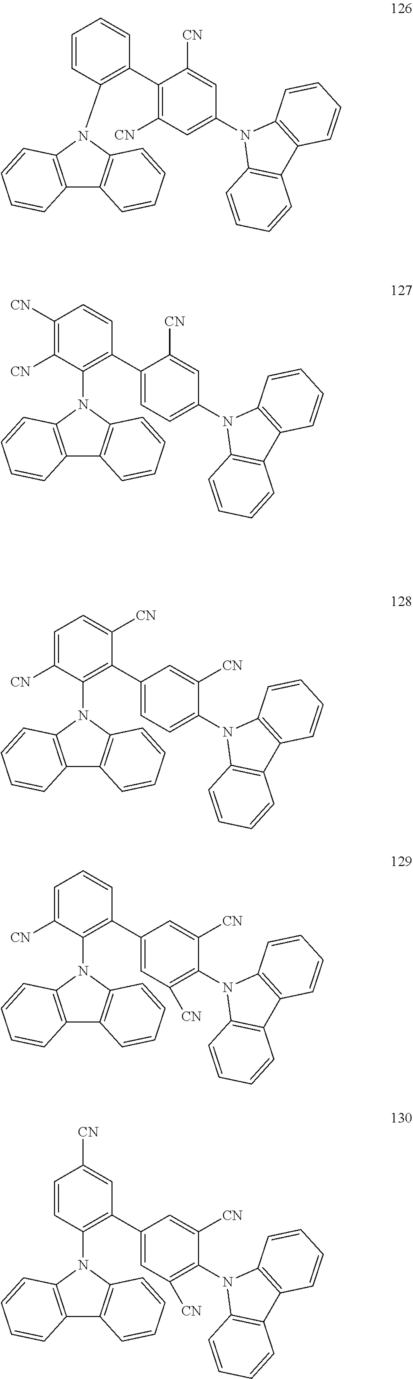

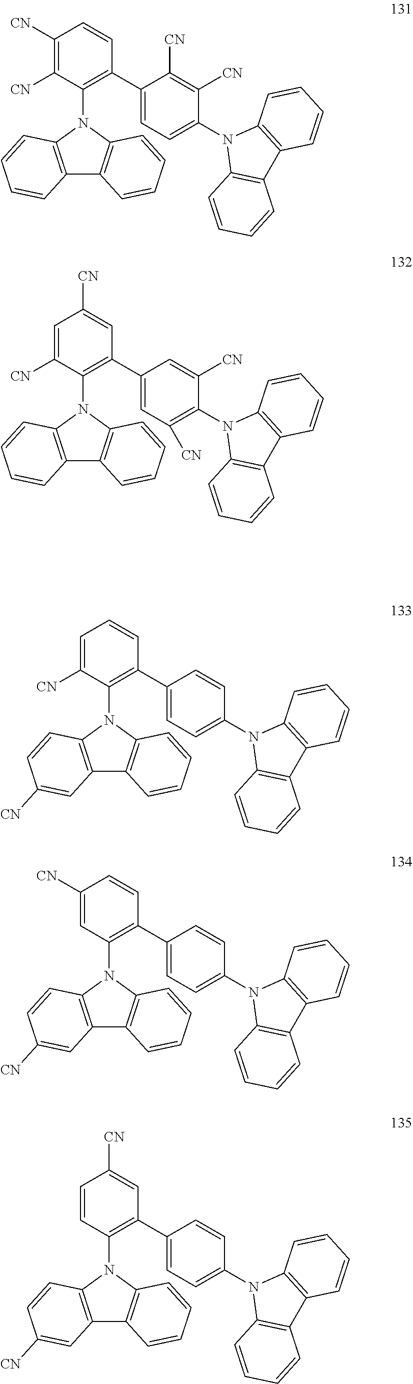

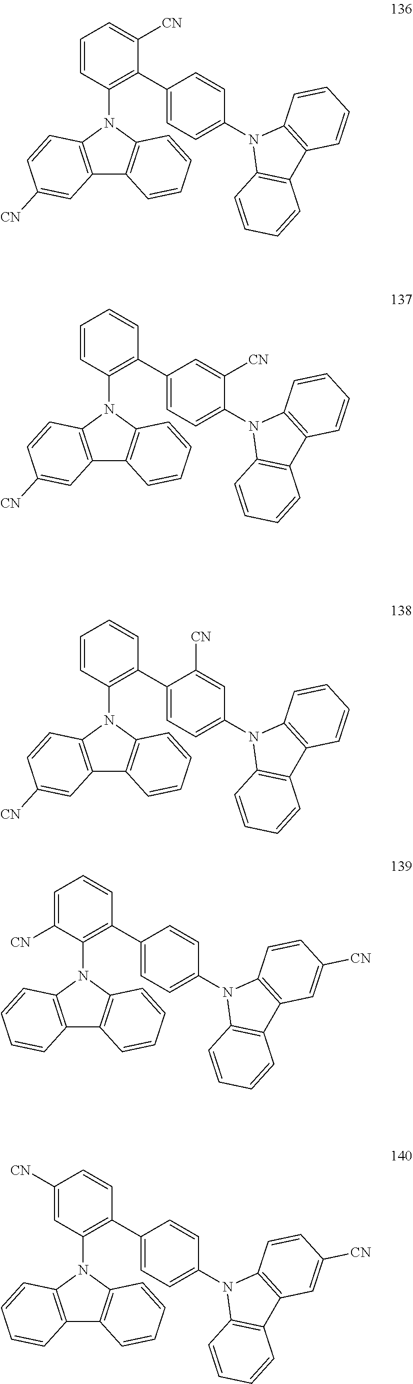

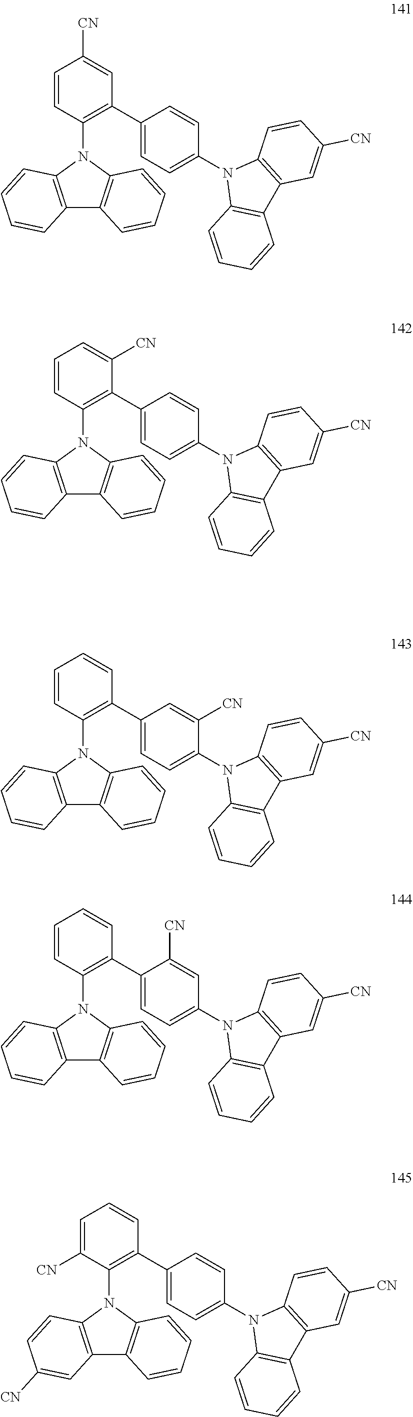

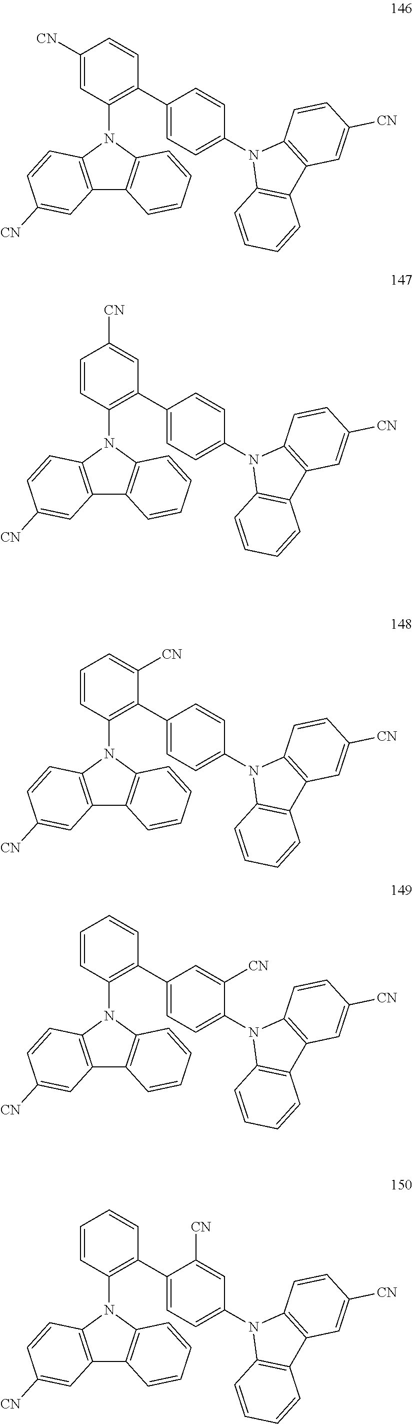

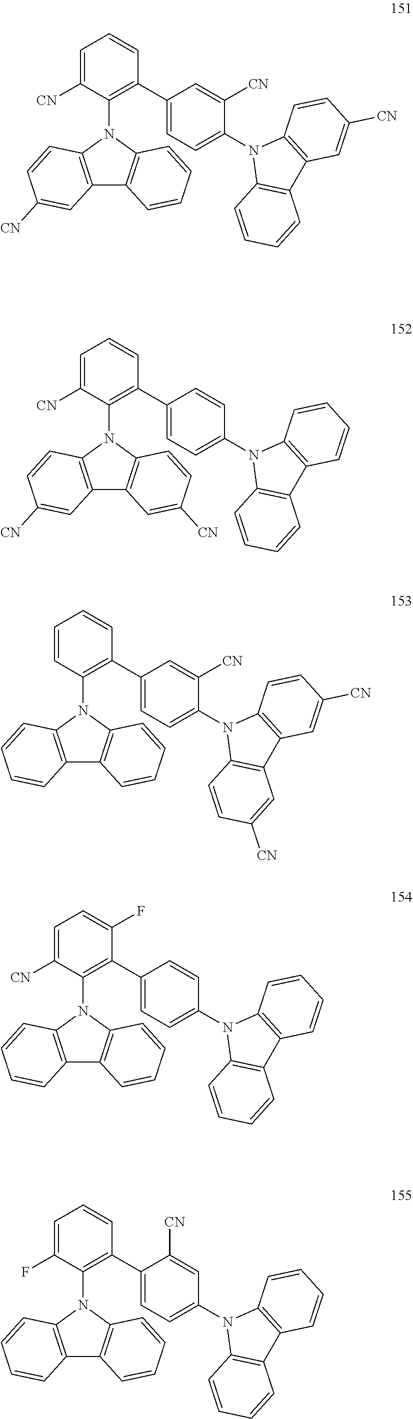

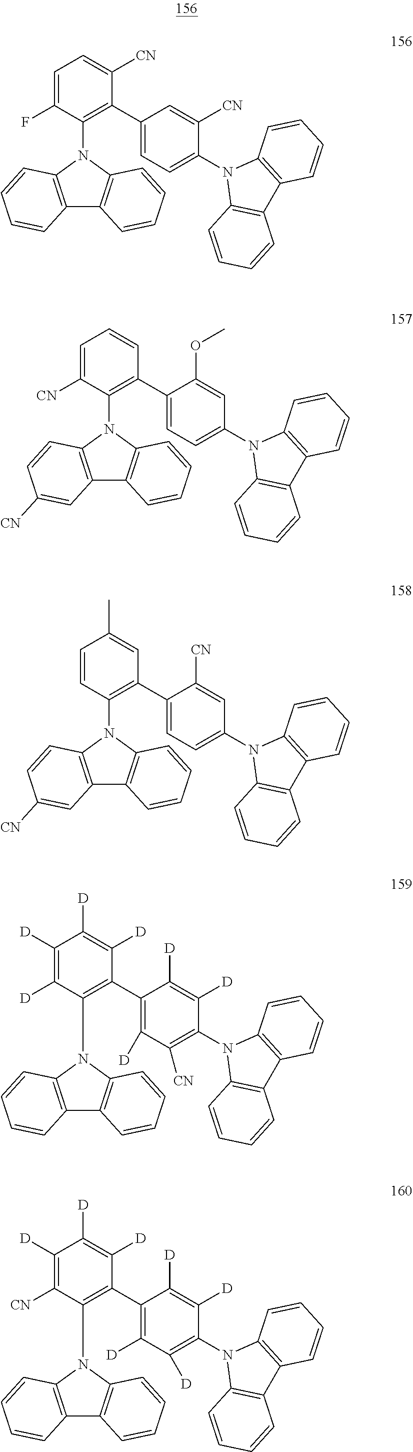

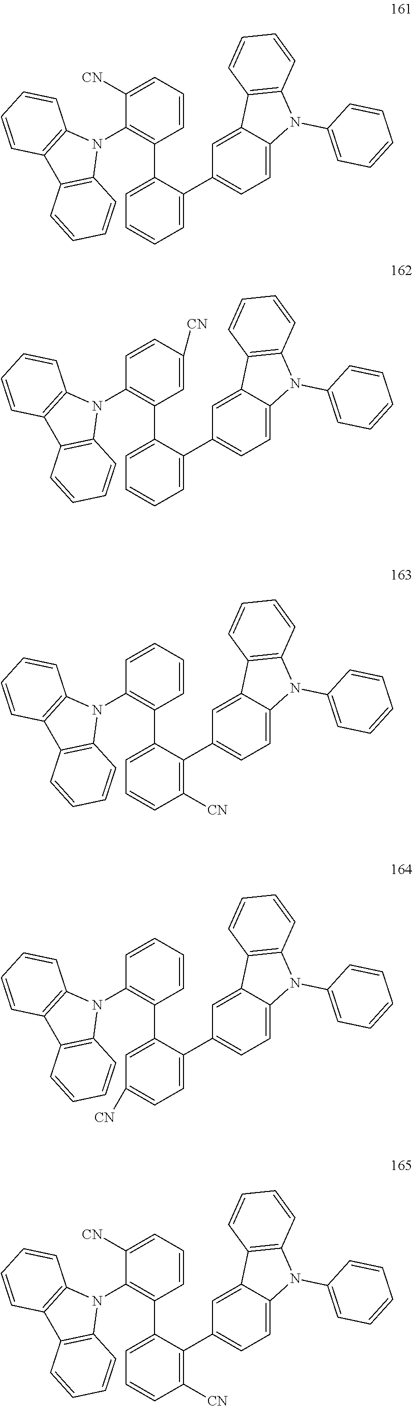

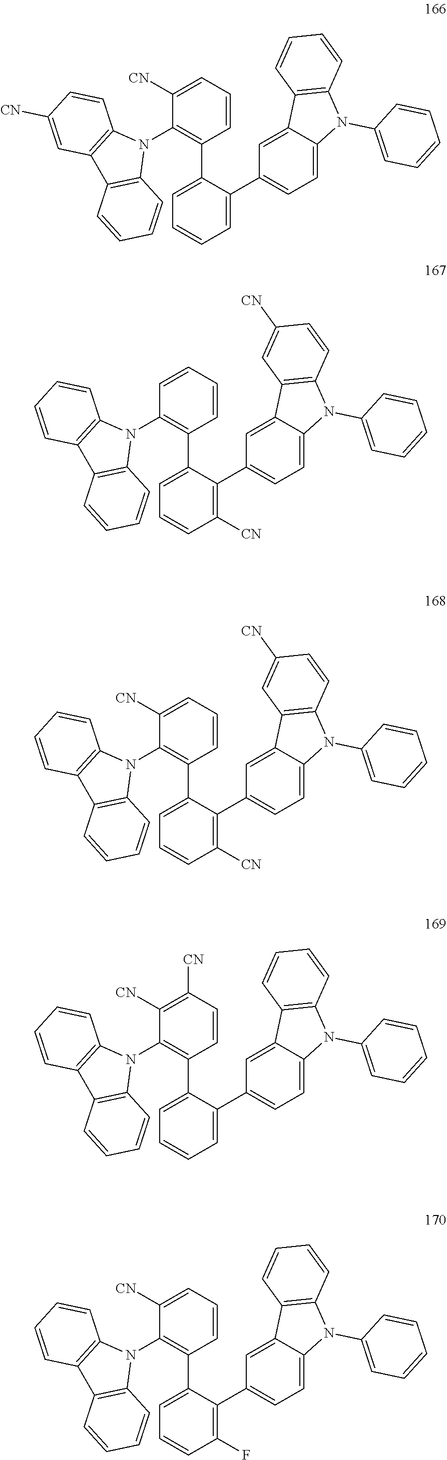

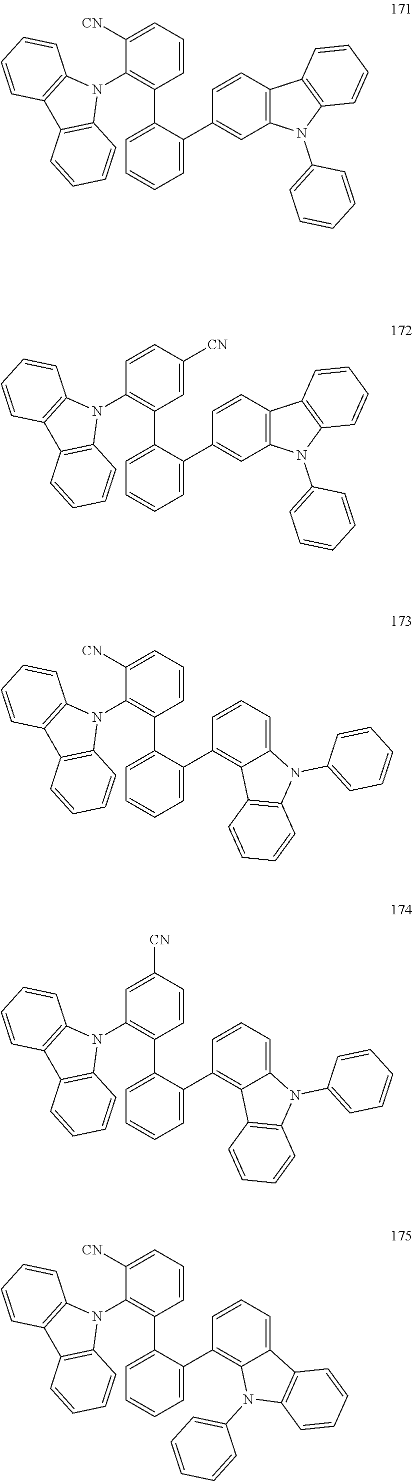

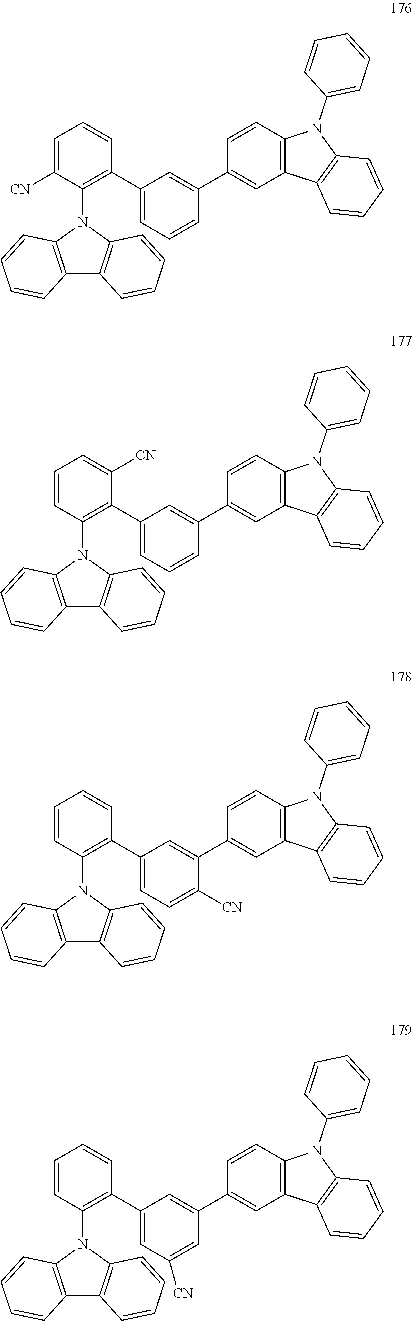

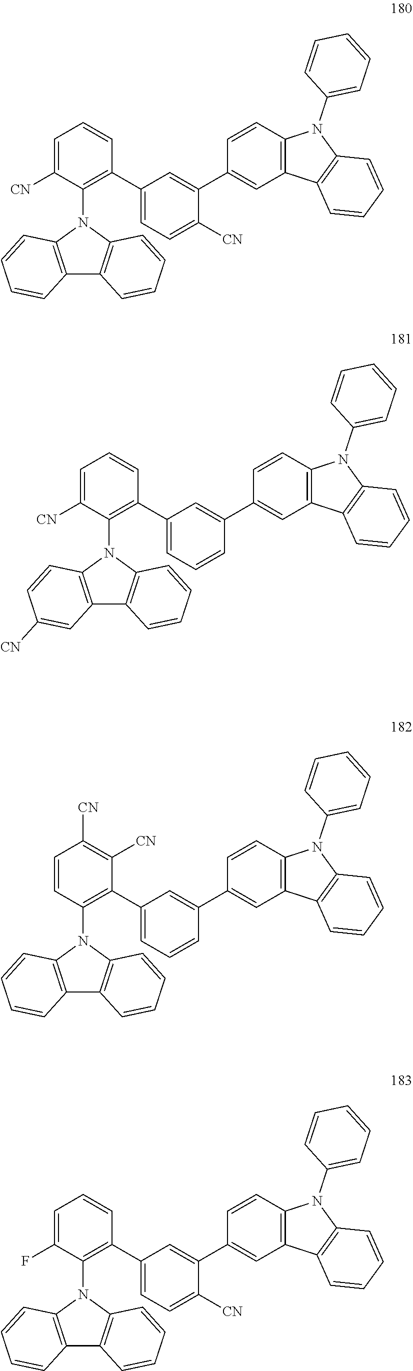

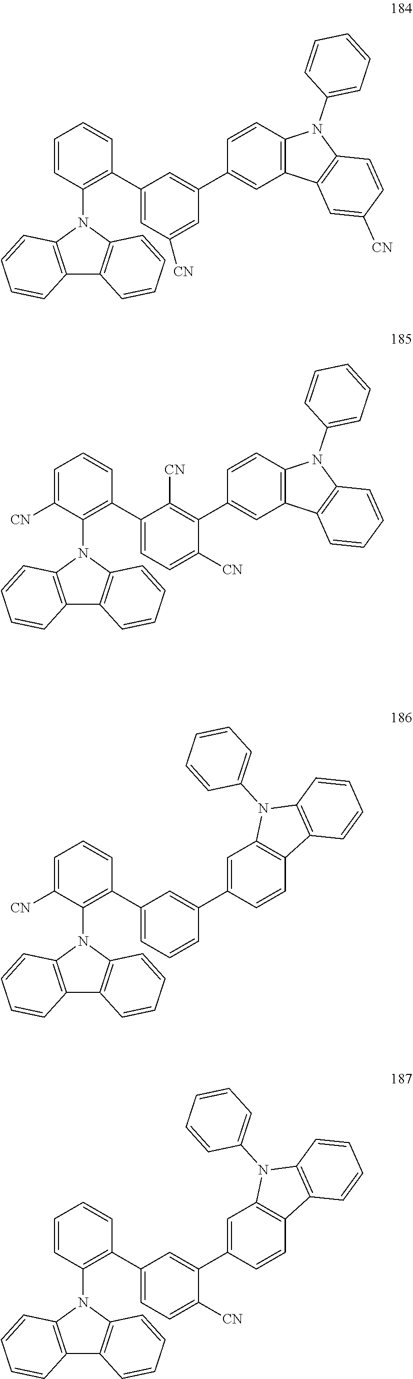

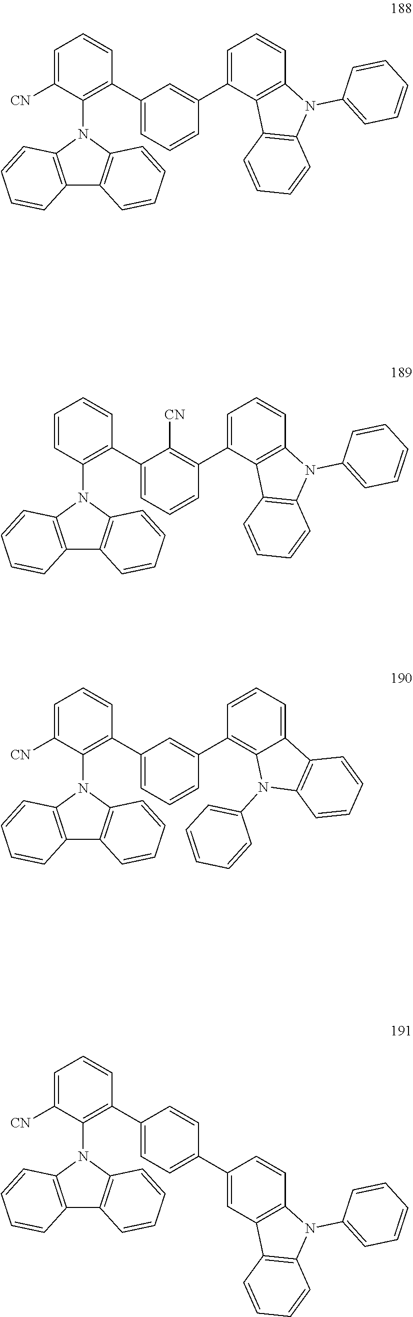

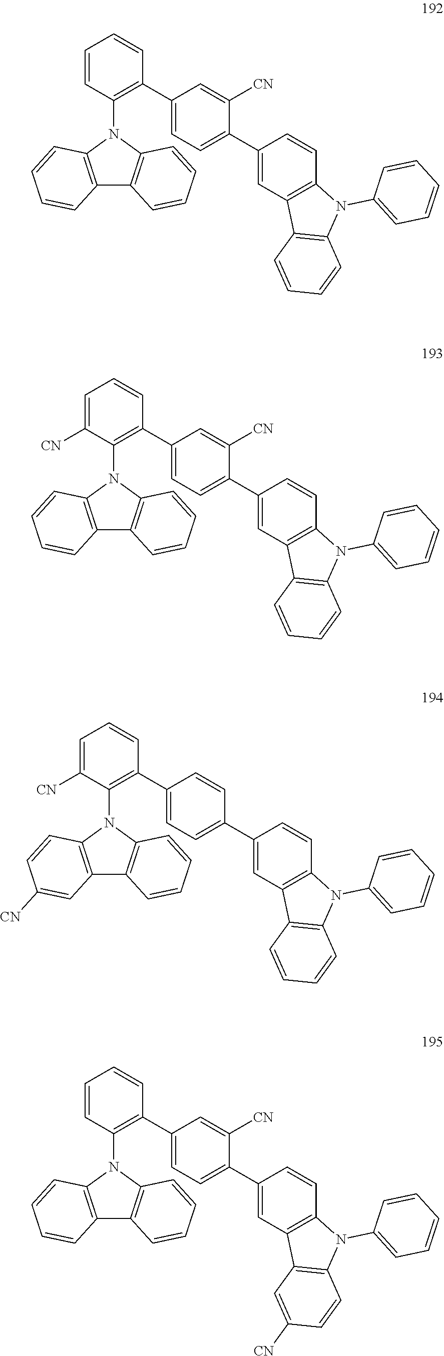

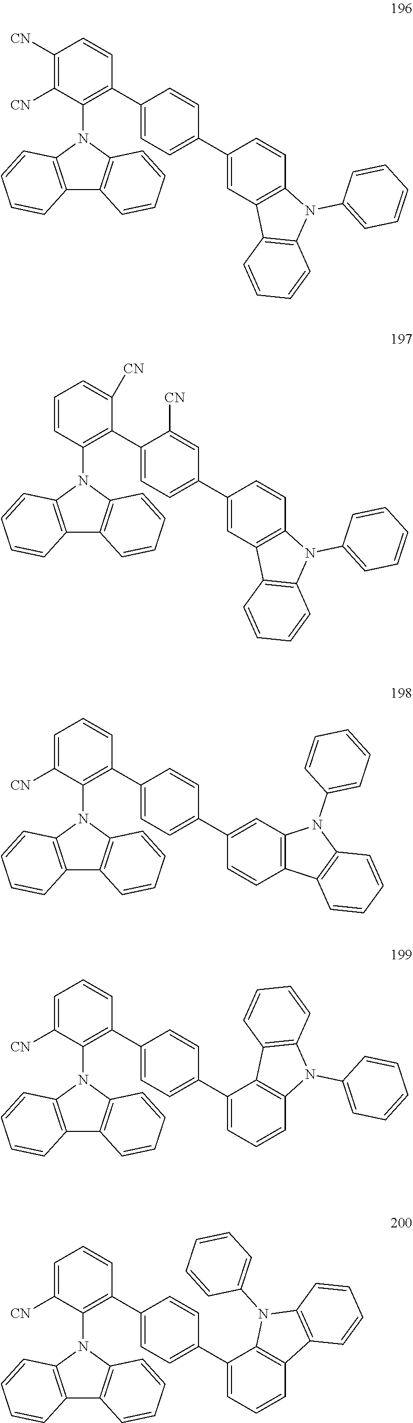

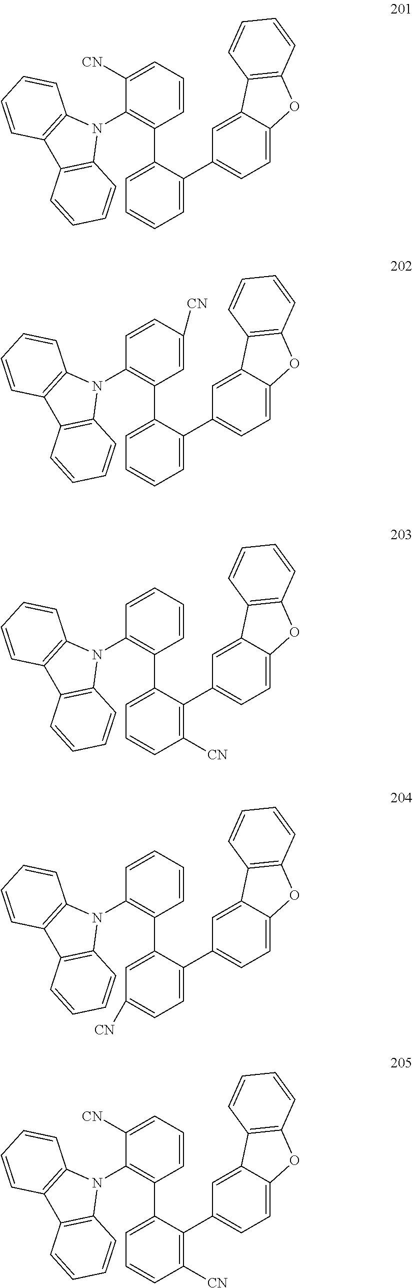

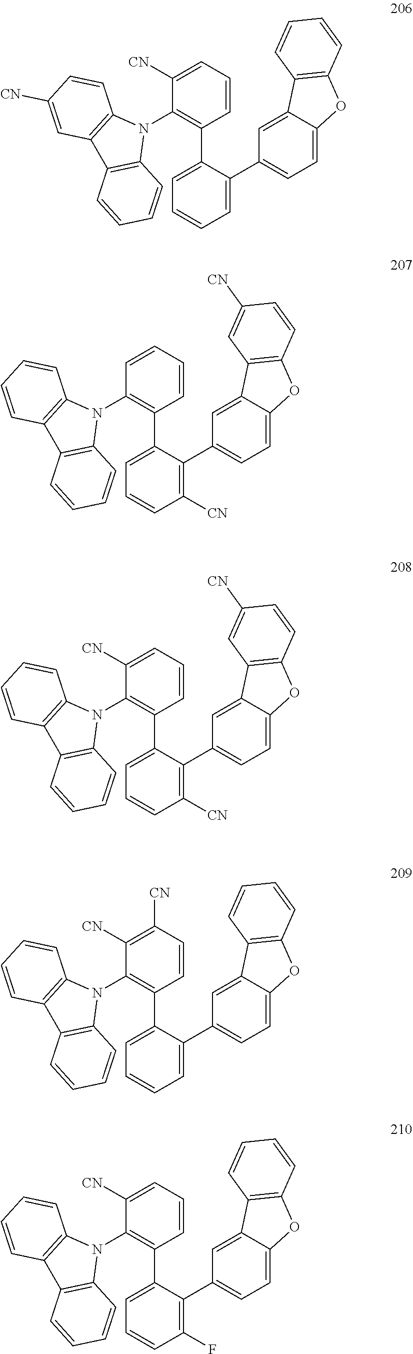

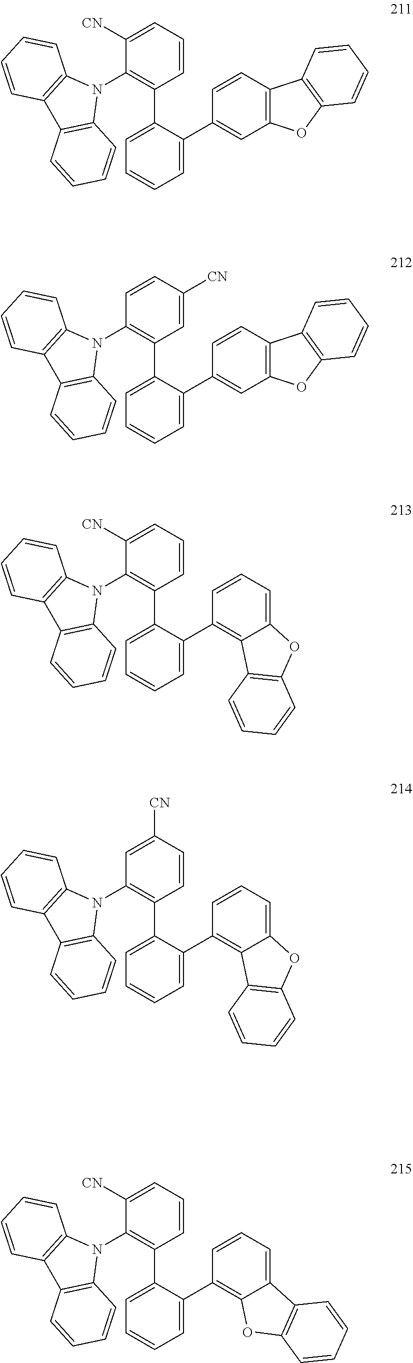

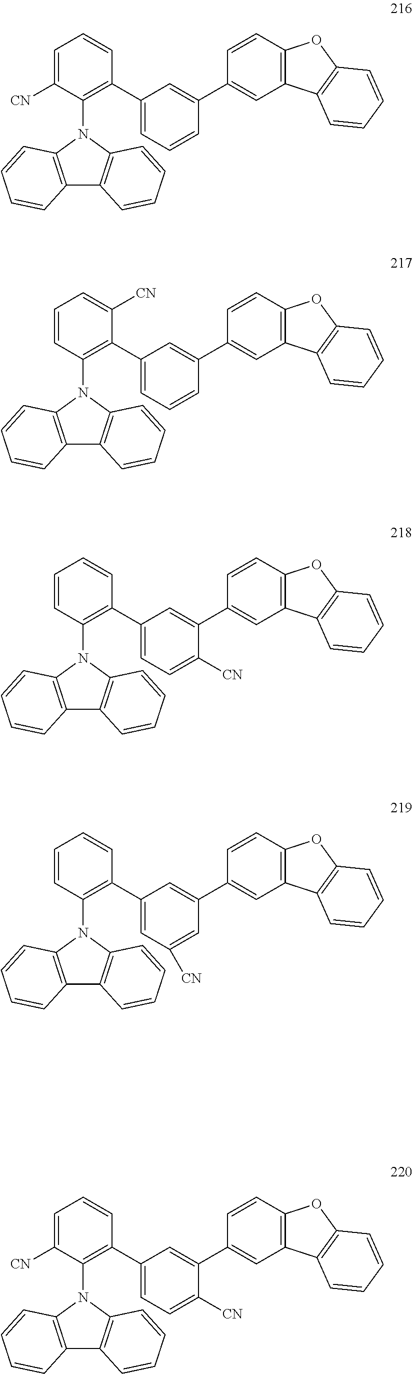

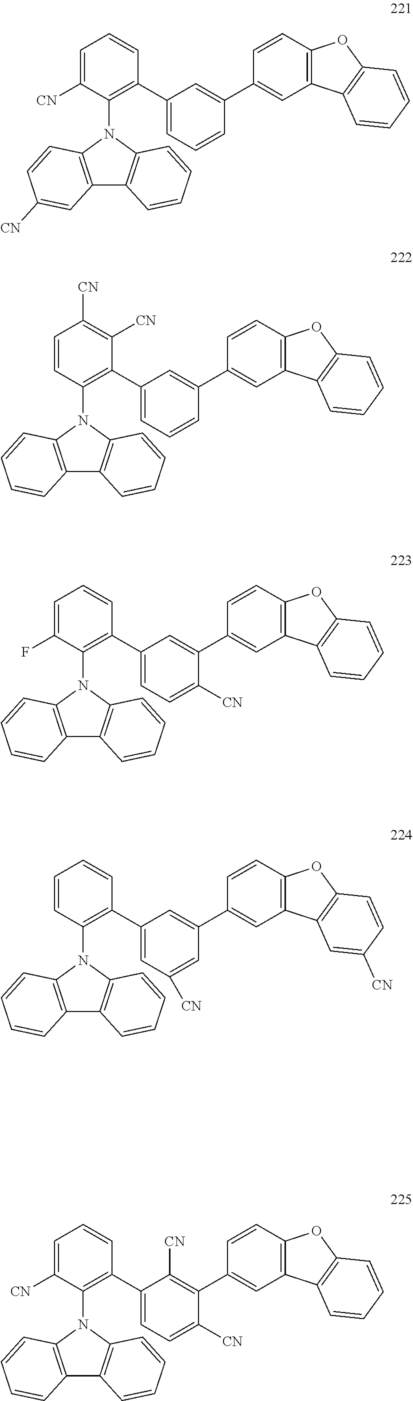

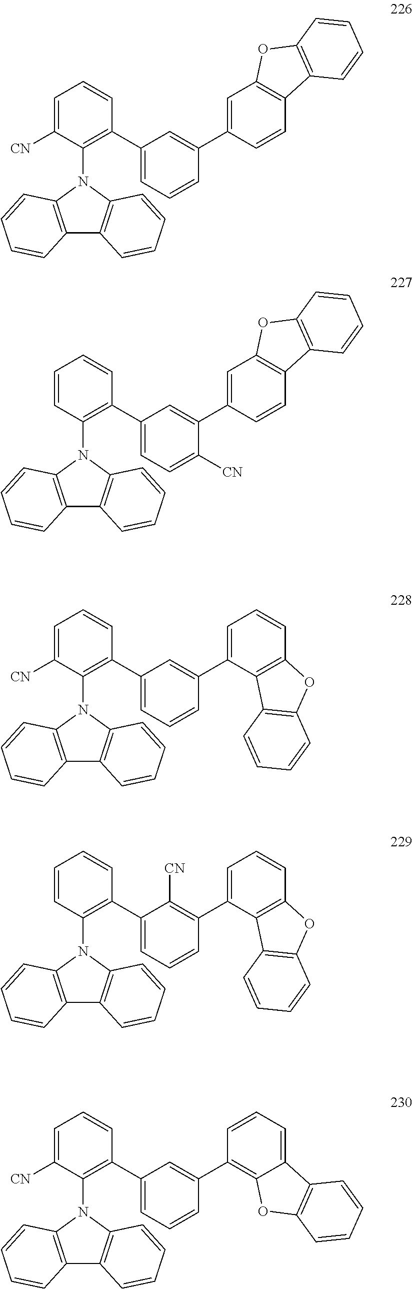

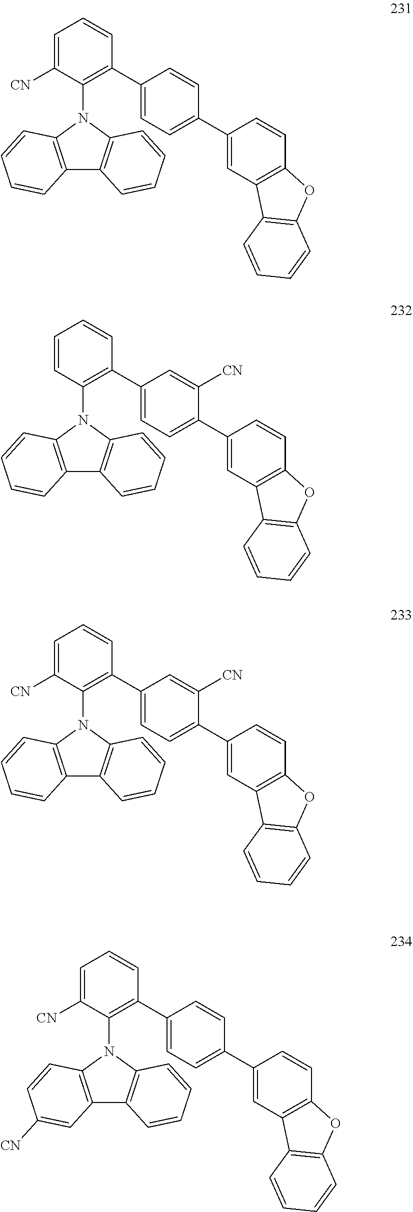

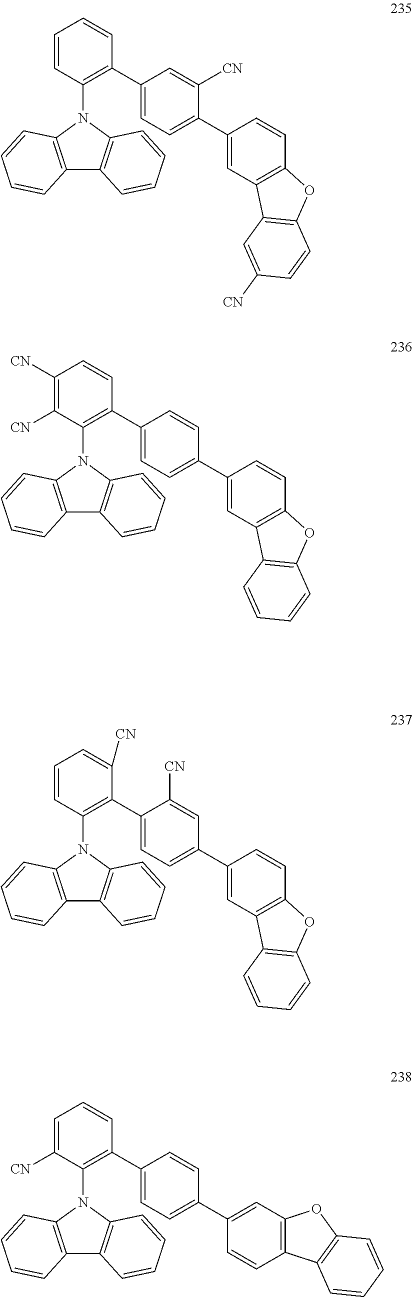

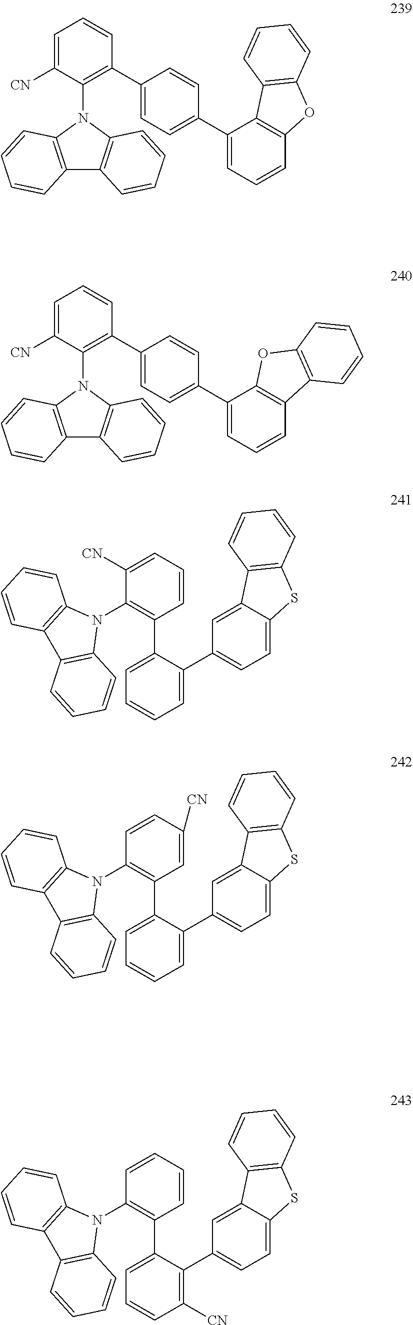

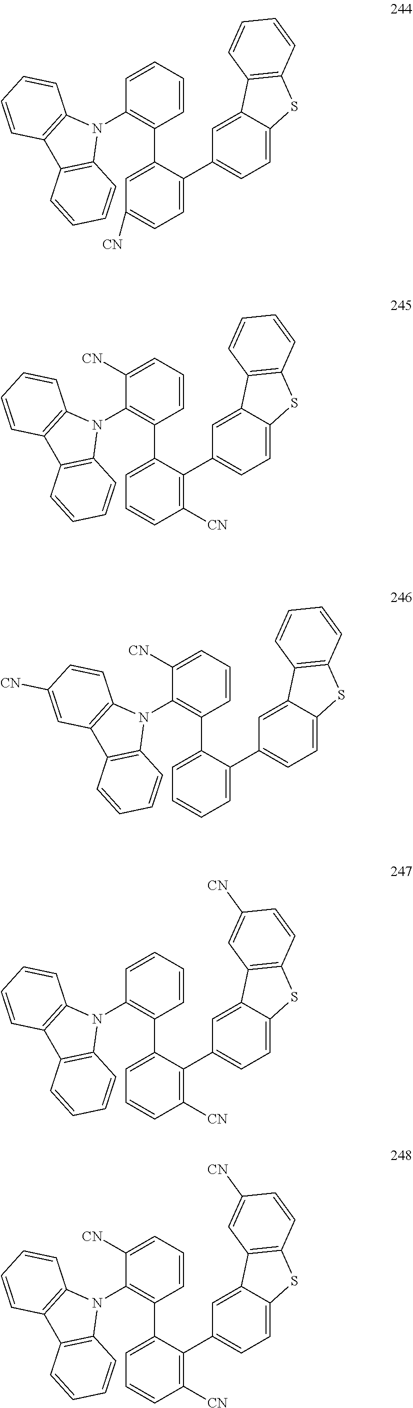

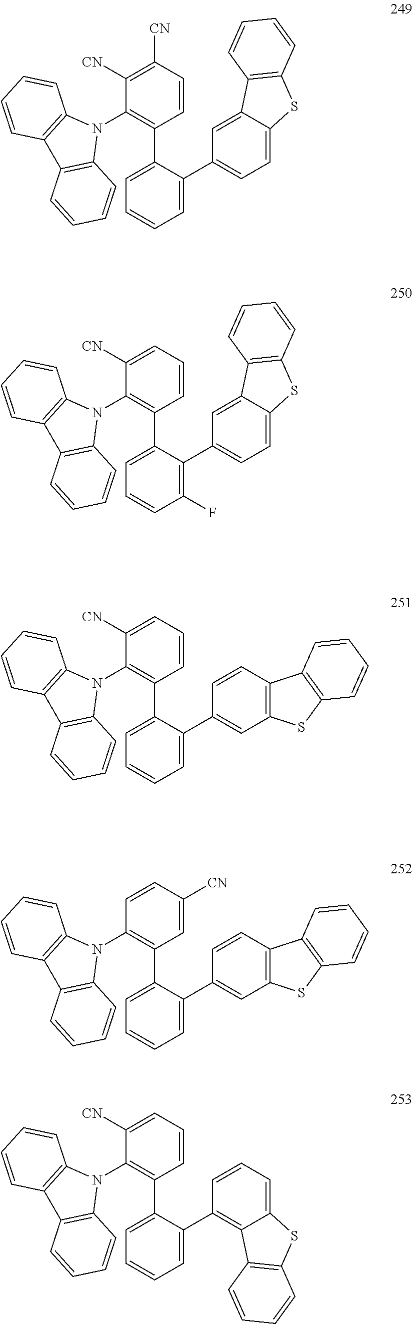

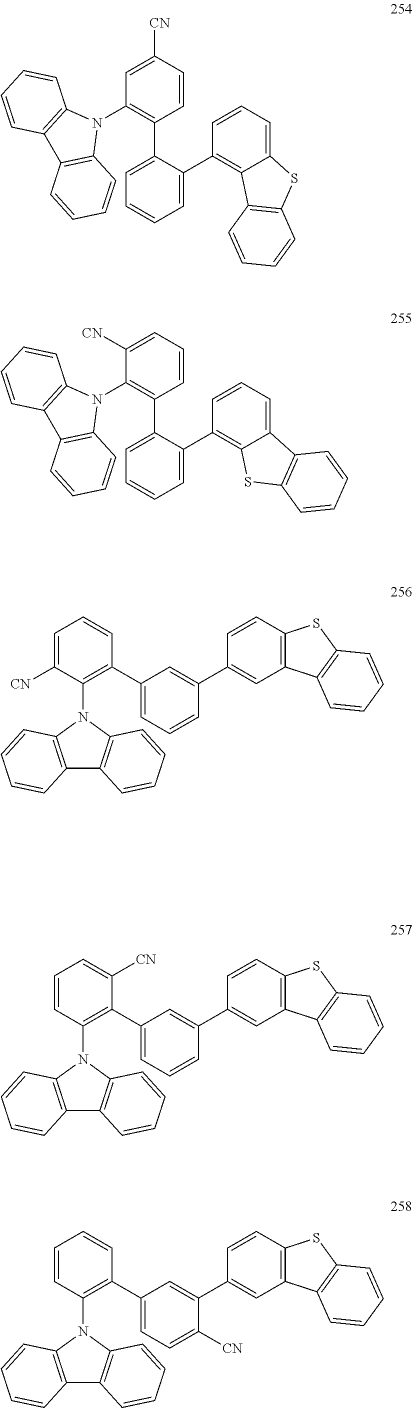

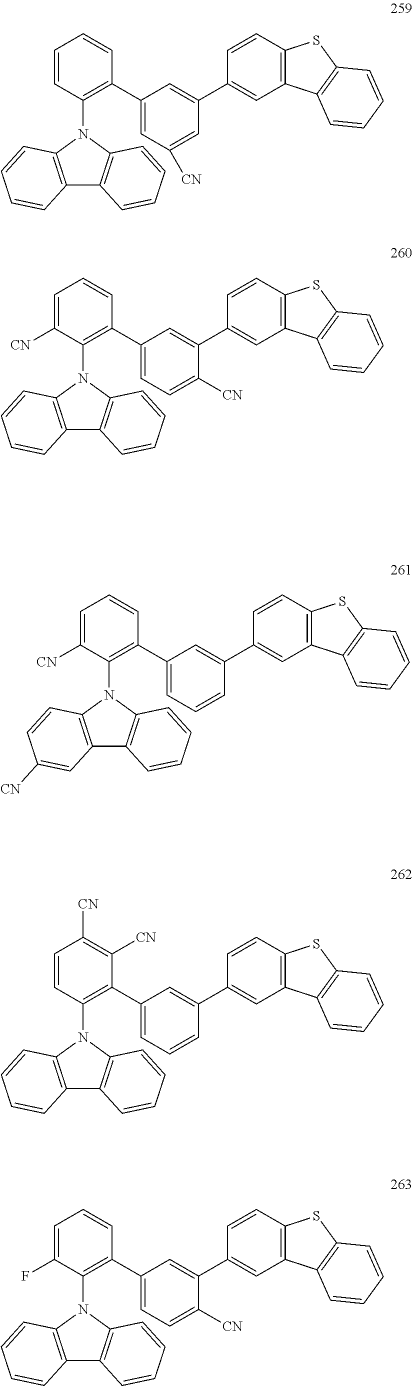

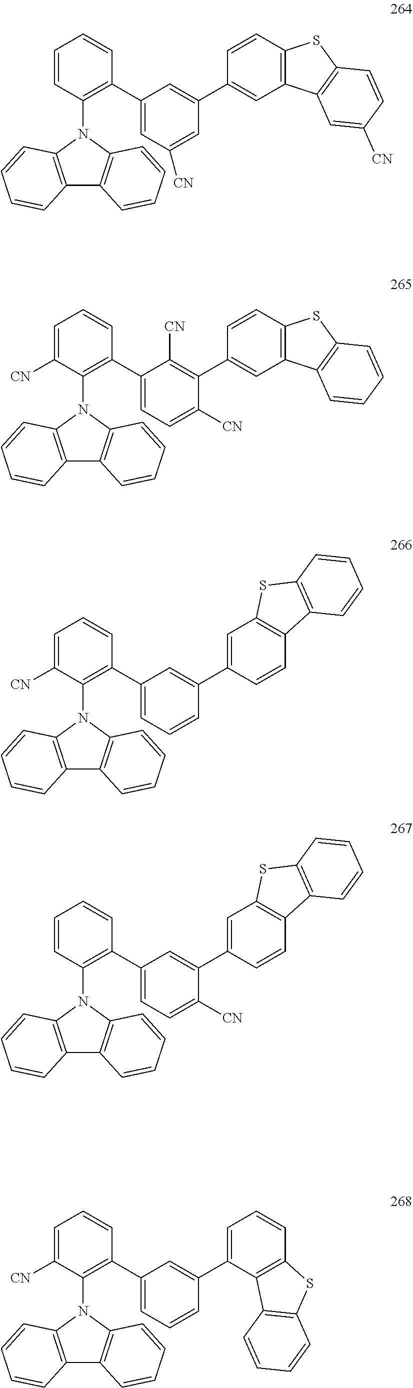

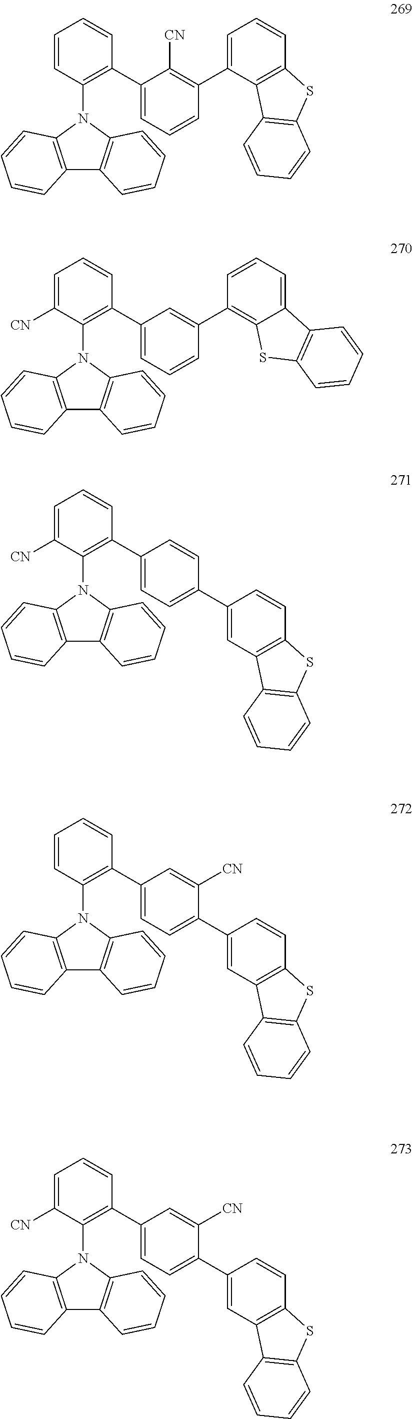

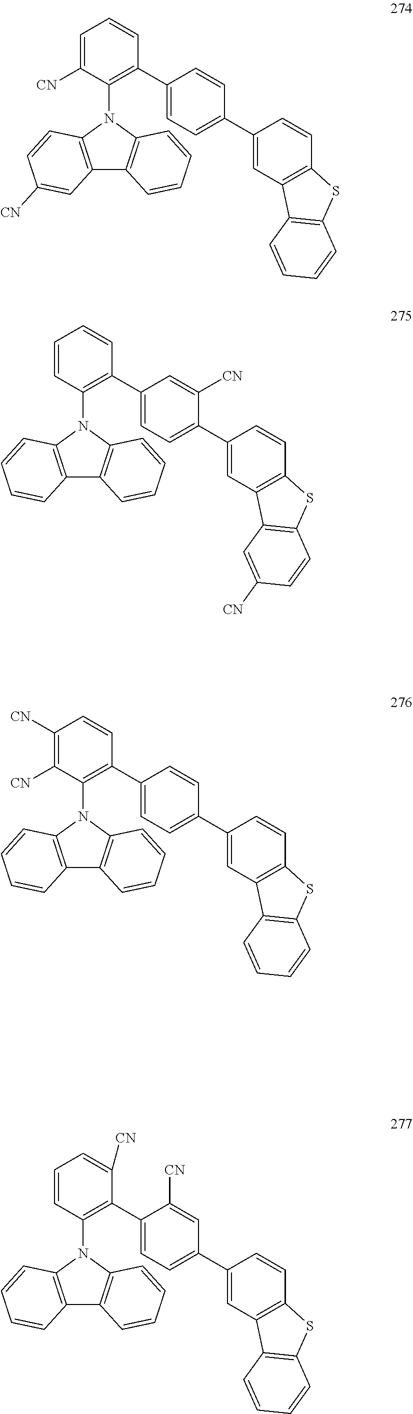

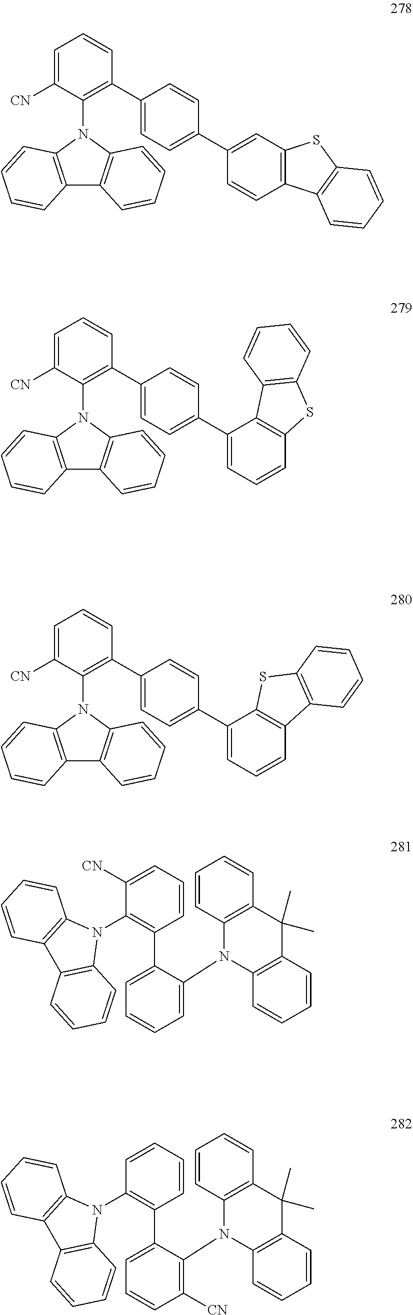

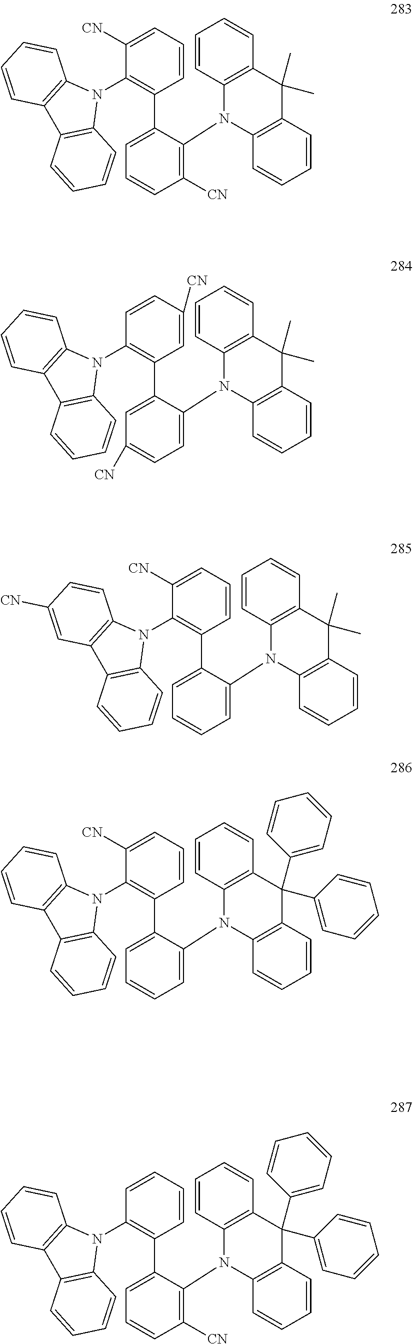

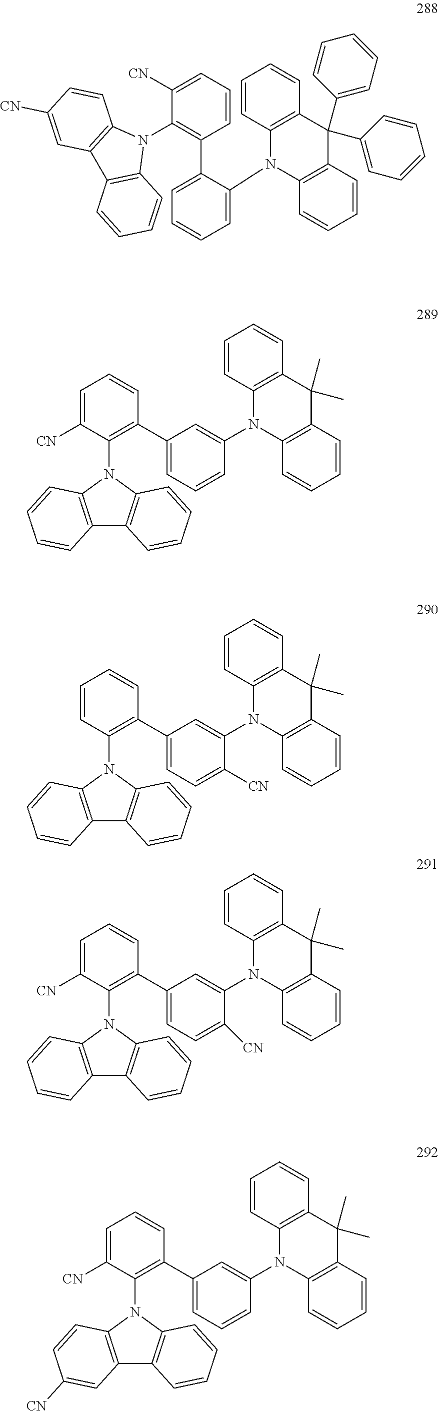

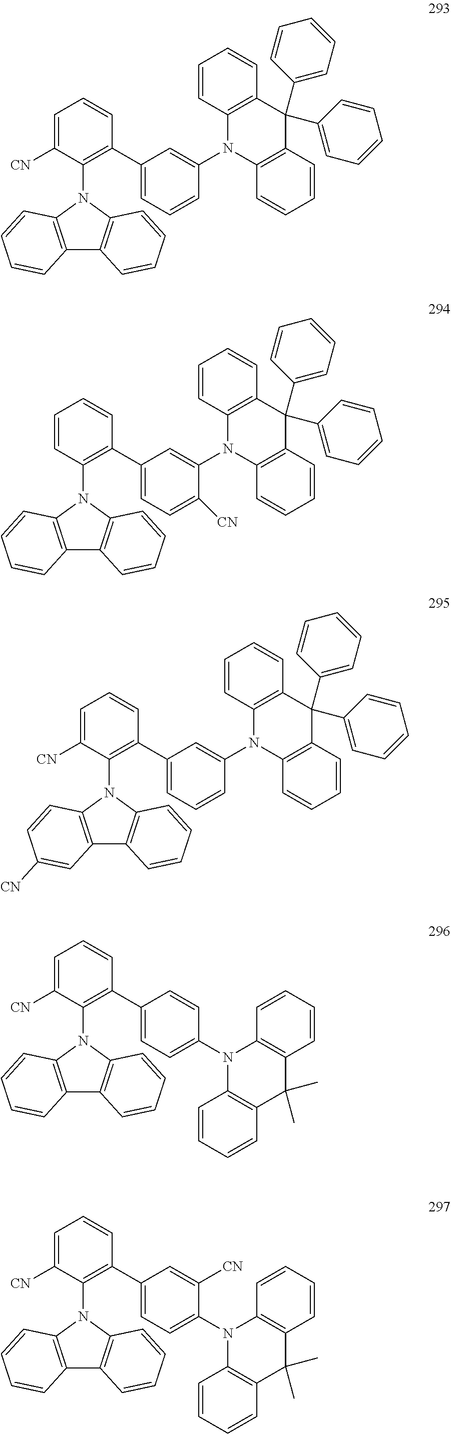

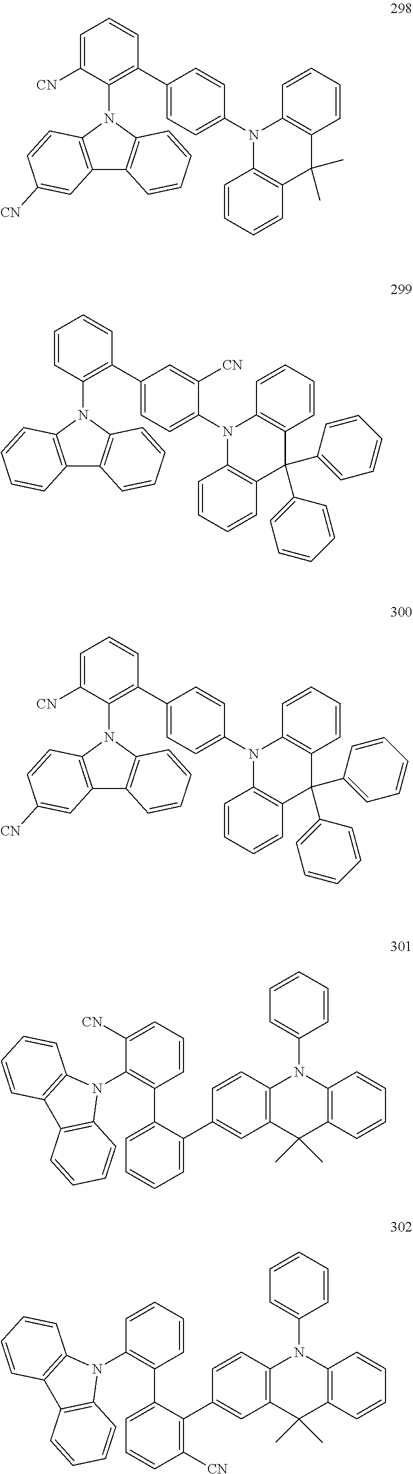

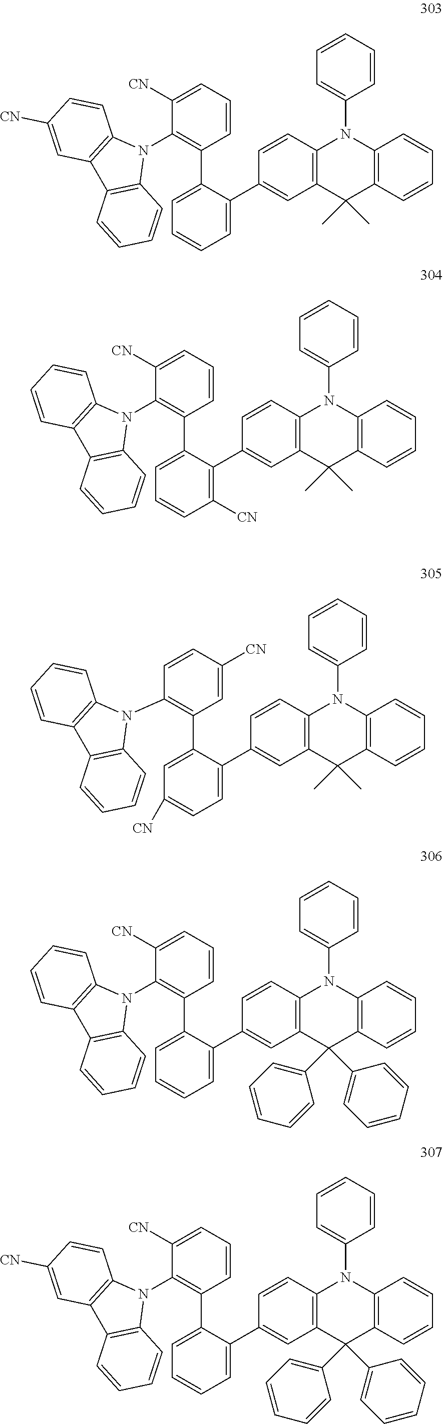

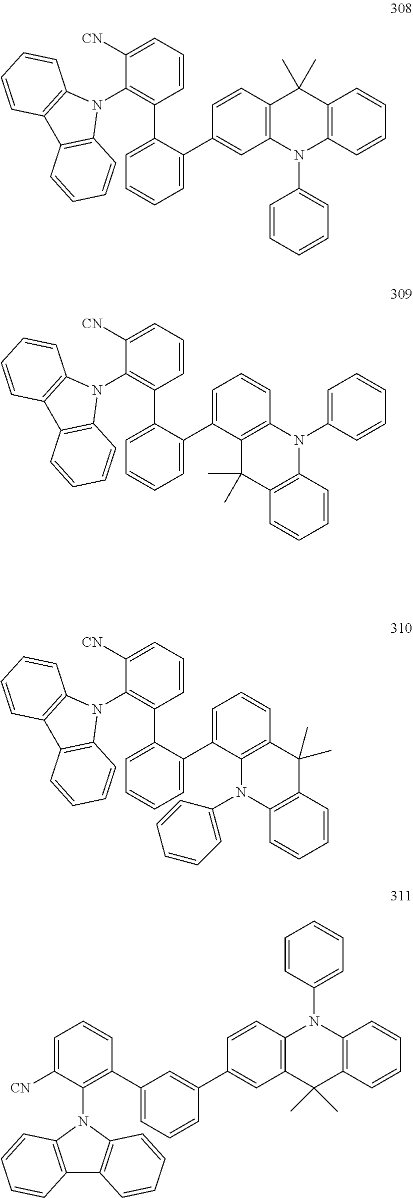

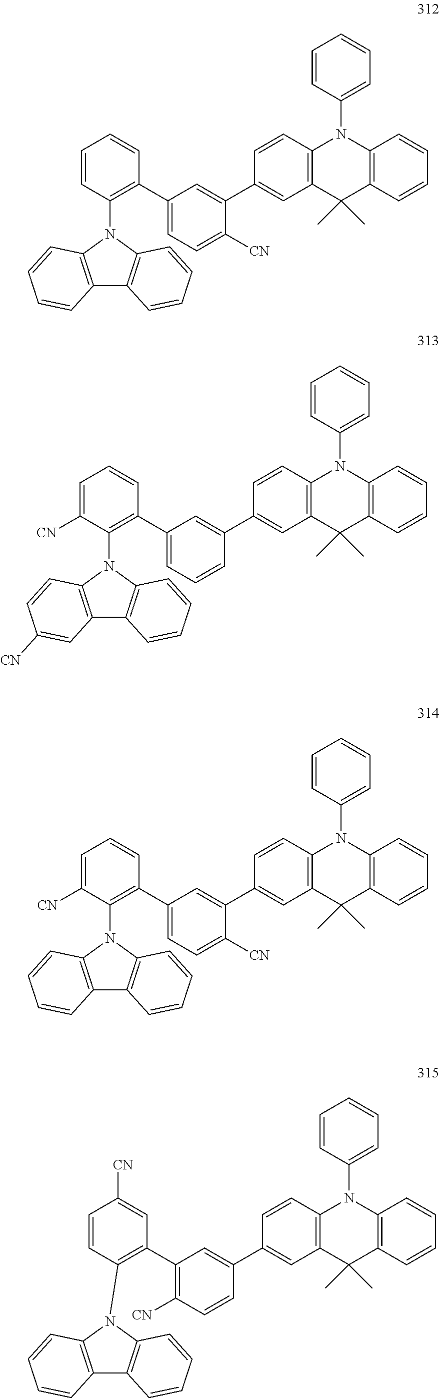

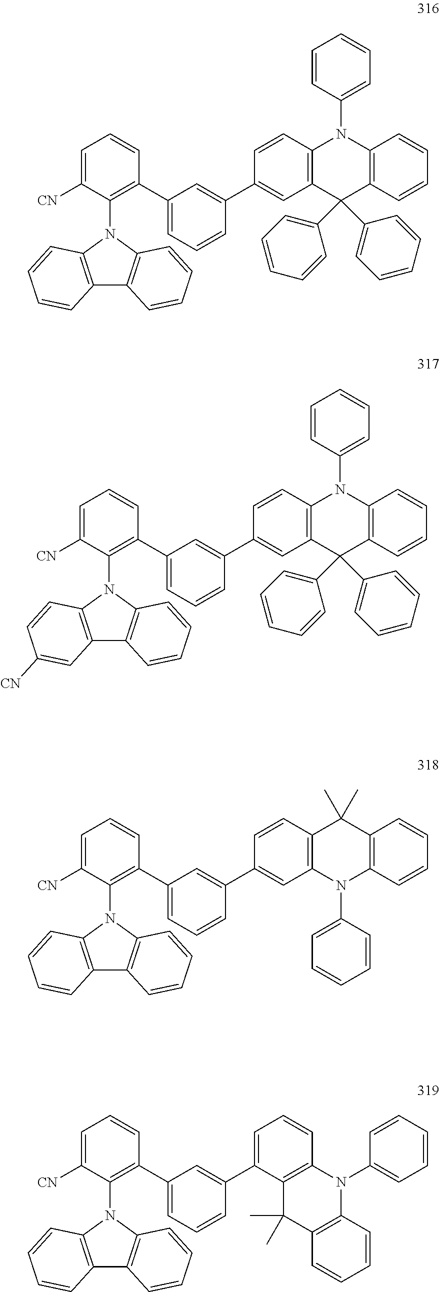

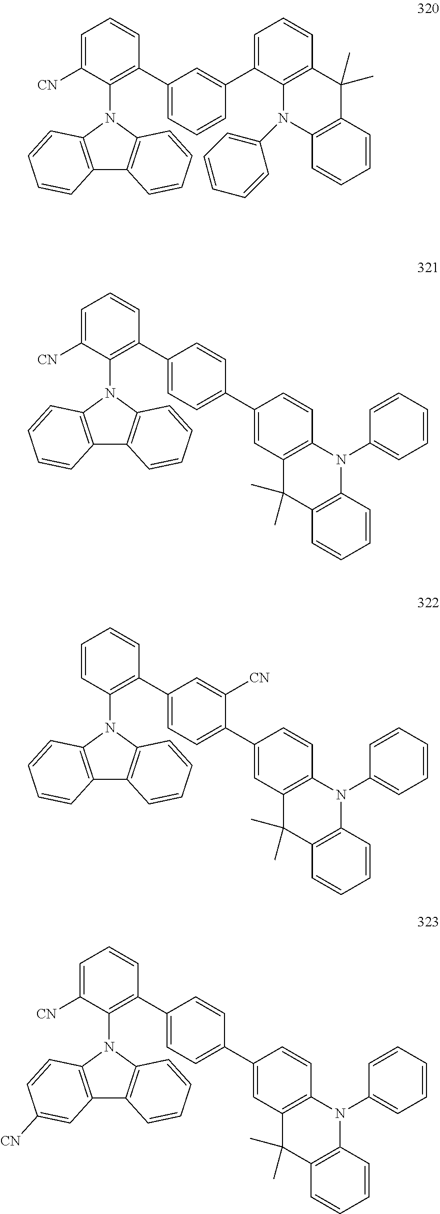

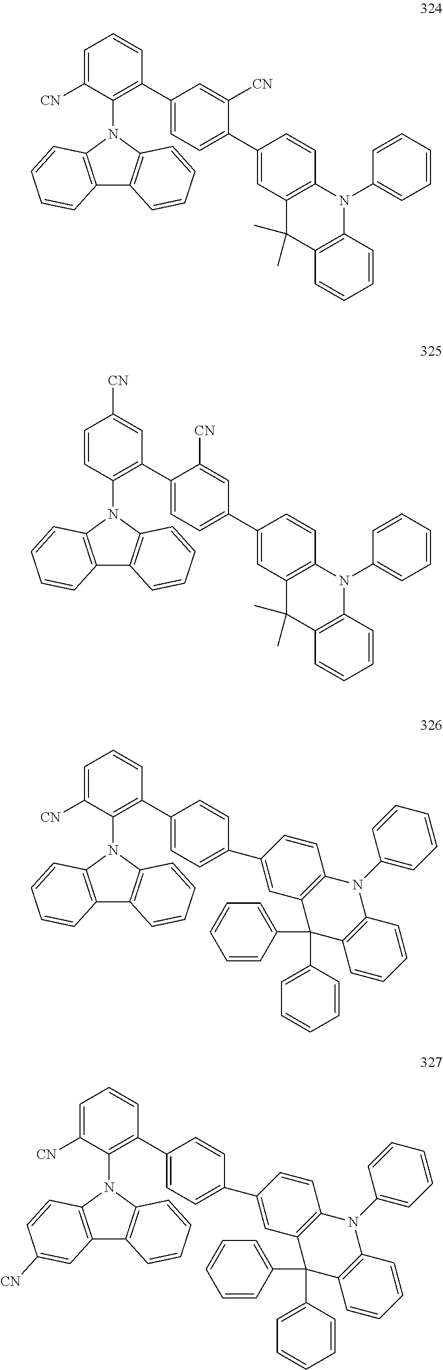

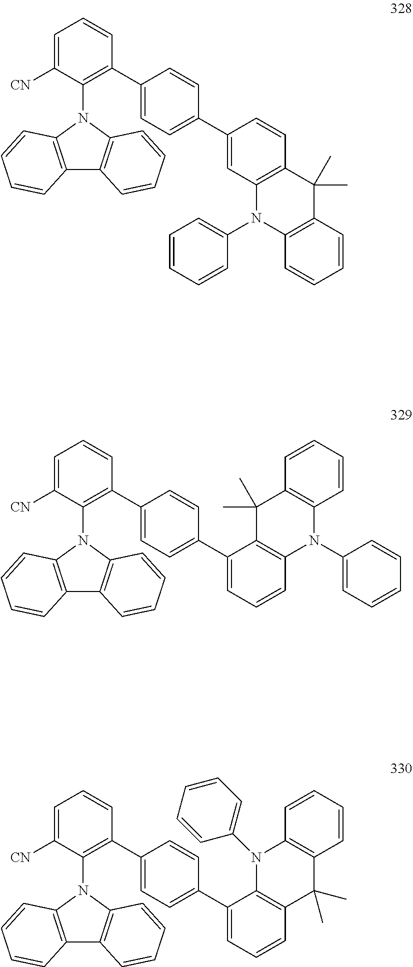

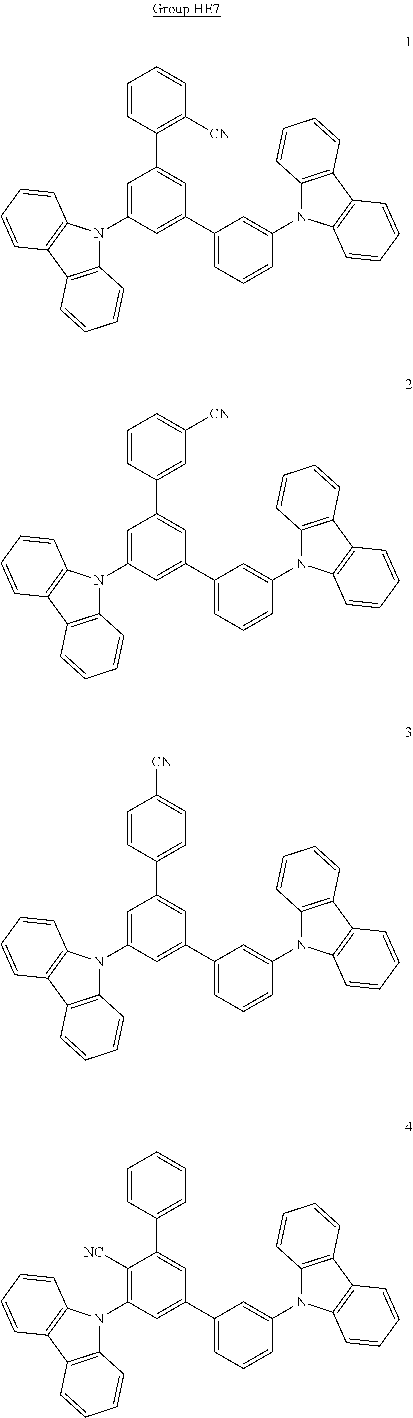 [SMILES: N#Cc1ccccc1-c1cc(-c2cccc(-n3c4ccccc4c4ccccc43)c2)cc(-n2c3ccccc3c3ccccc32)c1 ; N#Cc1cccc(-c2cc(-c3cccc(-n4c5ccccc5c5ccccc54)c3)cc(-n3c4ccccc4c4ccccc43)c2)c1 ; N#Cc1ccc(-c2cc(-c3cccc(-n4c5ccccc5c5ccccc54)c3)cc(-n3c4ccccc4c4ccccc43)c2)cc1 ; N#Cc1c(-c2ccccc2)cc(-c2cccc(-n3c4ccccc4c4ccccc43)c2)cc1-n1c2ccccc2c2ccccc21]

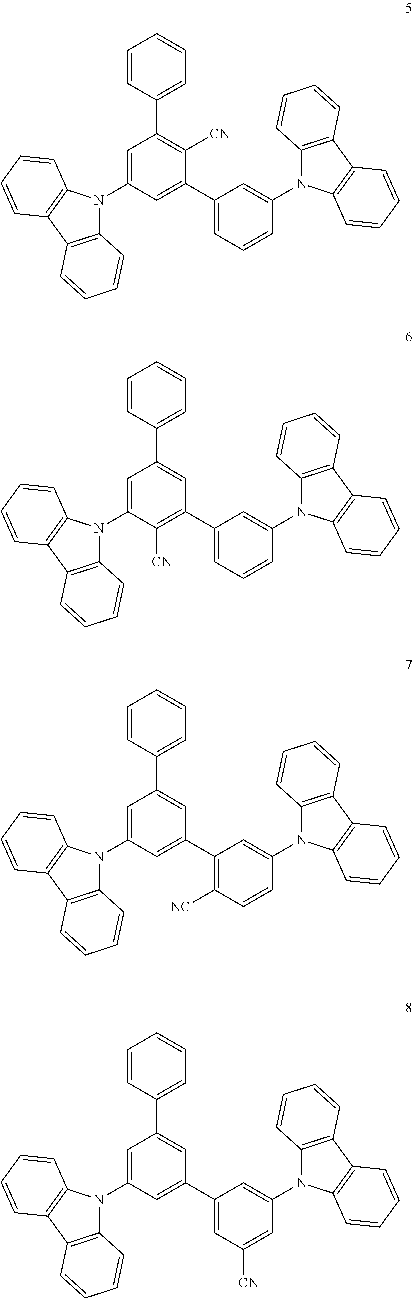 [SMILES: N#Cc1c(-c2ccccc2)cc(-n2c3ccccc3c3ccccc32)cc1-c1cccc(-n2c3ccccc3c3ccccc32)c1 ; N#Cc1c(-c2cccc(-n3c4ccccc4c4ccccc43)c2)cc(-c2ccccc2)cc1-n1c2ccccc2c2ccccc21 ; N#Cc1ccc(-n2c3ccccc3c3ccccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3ccccc32)c1]

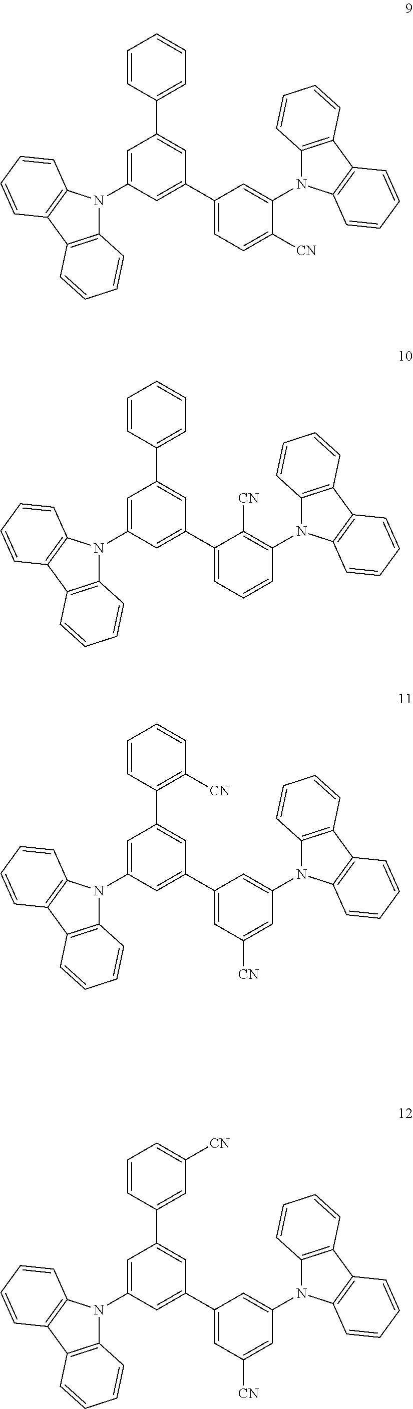 [SMILES: N#Cc1ccc(-c2cc(-c3ccccc3)cc(-n3c4ccccc4c4ccccc43)c2)cc1-n1c2ccccc2c2ccccc21 ; N#Cc1c(-c2cc(-c3ccccc3)cc(-n3c4ccccc4c4ccccc43)c2)cccc1-n1c2ccccc2c2ccccc21 ; N#Cc1cc(-c2cc(-c3ccccc3C#N)cc(-n3c4ccccc4c4ccccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4ccccc43)c2)c1]

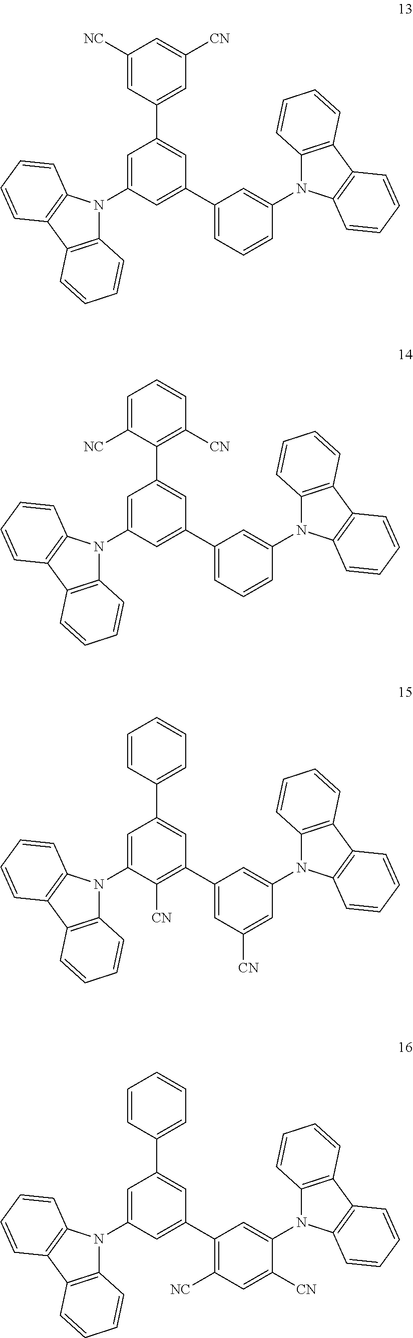 [SMILES: N#Cc1cc(C#N)cc(-c2cc(-c3cccc(-n4c5ccccc5c5ccccc54)c3)cc(-n3c4ccccc4c4ccccc43)c2)c1 ; N#Cc1cccc(C#N)c1-c1cc(-c2cccc(-n3c4ccccc4c4ccccc43)c2)cc(-n2c3ccccc3c3ccccc32)c1 ; N#Cc1cc(-c2cc(-c3ccccc3)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2ccccc2)cc(-n2c3ccccc3c3ccccc32)c1]

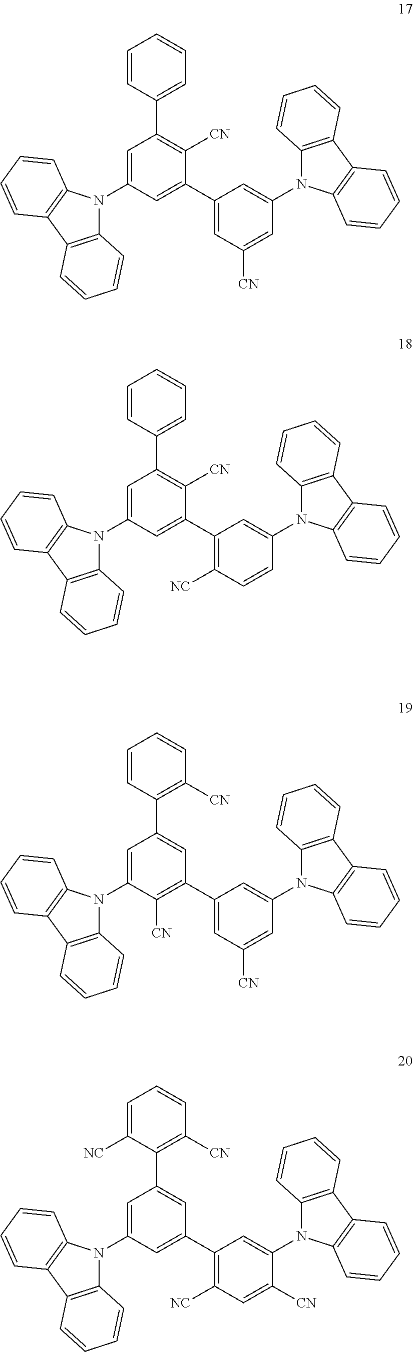 [SMILES: N#Cc1cc(-c2cc(-n3c4ccccc4c4ccccc43)cc(-c3ccccc3)c2C#N)cc(-n2c3ccccc3c3ccccc32)c1 ; N#Cc1ccc(-n2c3ccccc3c3ccccc32)cc1-c1cc(-n2c3ccccc3c3ccccc32)cc(-c2ccccc2)c1C#N ; N#Cc1cc(-c2cc(-c3ccccc3C#N)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2c(C#N)cccc2C#N)cc(-n2c3ccccc3c3ccccc32)c1]

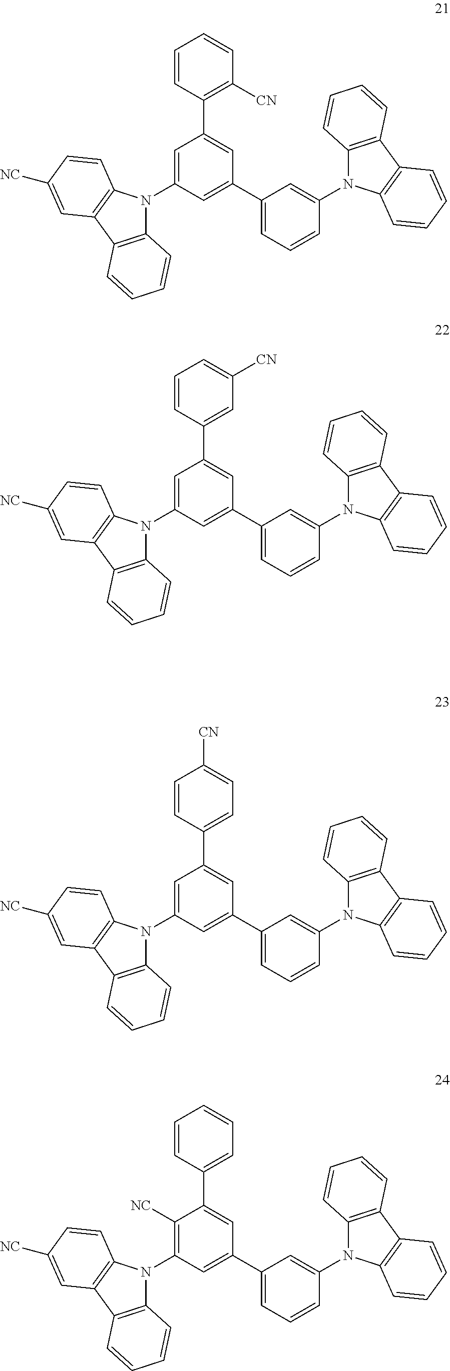 [SMILES: N#Cc1ccc2c(c1)c1ccccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccccc4c4cc(C#N)ccc43)c2)c1 ; N#Cc1ccc(-c2cc(-c3cccc(-n4c5ccccc5c5ccccc54)c3)cc(-n3c4ccccc4c4cc(C#N)ccc43)c2)cc1 ; N#Cc1ccc2c(c1)c1ccccc1n2-c1cc(-c2cccc(-n3c4ccccc4c4ccccc43)c2)cc(-c2ccccc2)c1C#N]

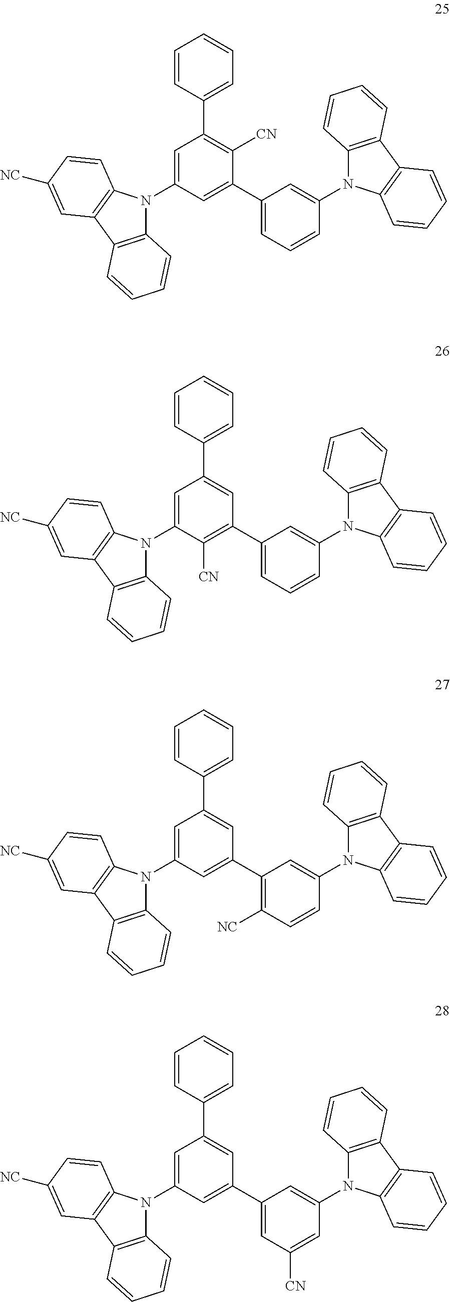 [SMILES: N#Cc1ccc2c(c1)c1ccccc1n2-c1cc(-c2ccccc2)c(C#N)c(-c2cccc(-n3c4ccccc4c4ccccc43)c2)c1 ; N#Cc1ccc2c(c1)c1ccccc1n2-c1cc(-c2ccccc2)cc(-c2cccc(-n3c4ccccc4c4ccccc43)c2)c1C#N ; N#Cc1ccc2c(c1)c1ccccc1n2-c1cc(-c2ccccc2)cc(-c2cc(-n3c4ccccc4c4ccccc43)ccc2C#N)c1 ; N#Cc1cc(-c2cc(-c3ccccc3)cc(-n3c4ccccc4c4cc(C#N)ccc43)c2)cc(-n2c3ccccc3c3ccccc32)c1]

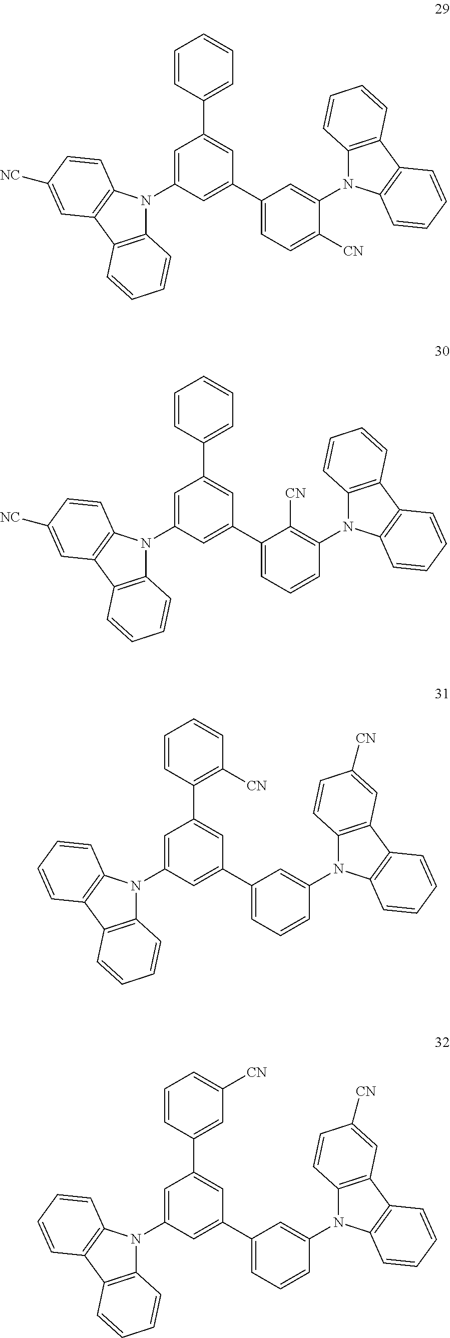 [SMILES: N#Cc1ccc2c(c1)c1ccccc1n2-c1cc(-c2ccccc2)cc(-c2ccc(C#N)c(-n3c4ccccc4c4ccccc43)c2)c1 ; N#Cc1ccc2c(c1)c1ccccc1n2-c1cc(-c2ccccc2)cc(-c2cccc(-n3c4ccccc4c4ccccc43)c2C#N)c1 ; N#Cc1ccc2c(c1)c1ccccc1n2-c1cccc(-c2cc(-c3ccccc3C#N)cc(-n3c4ccccc4c4ccccc43)c2)c1 ; N#Cc1cccc(-c2cc(-c3cccc(-n4c5ccccc5c5cc(C#N)ccc54)c3)cc(-n3c4ccccc4c4ccccc43)c2)c1]

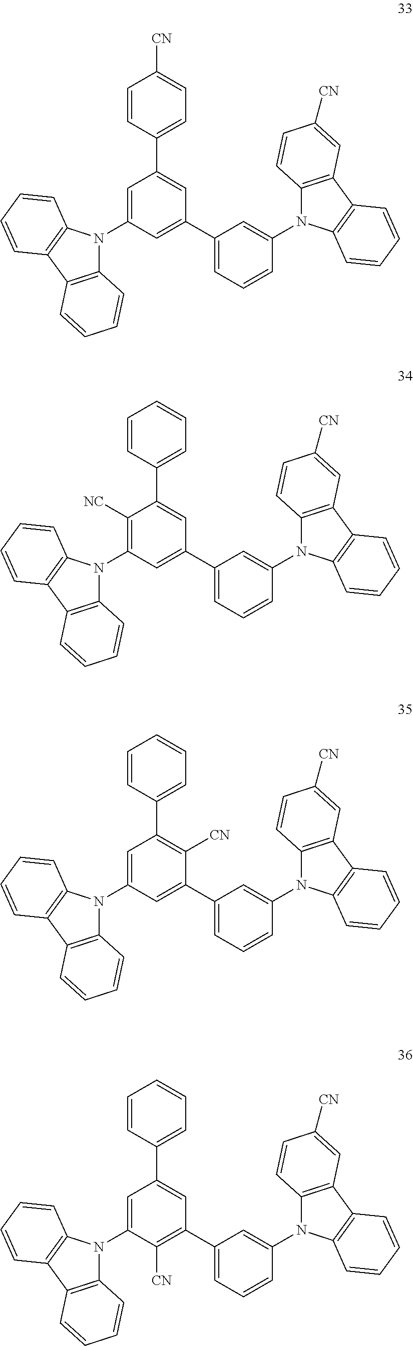 [SMILES: N#Cc1ccc(-c2cc(-c3cccc(-n4c5ccccc5c5cc(C#N)ccc54)c3)cc(-n3c4ccccc4c4ccccc43)c2)cc1 ; N#Cc1ccc2c(c1)c1ccccc1n2-c1cccc(-c2cc(-c3ccccc3)c(C#N)c(-n3c4ccccc4c4ccccc43)c2)c1 ; N#Cc1ccc2c(c1)c1ccccc1n2-c1cccc(-c2cc(-n3c4ccccc4c4ccccc43)cc(-c3ccccc3)c2C#N)c1 ; N#Cc1ccc2c(c1)c1ccccc1n2-c1cccc(-c2cc(-c3ccccc3)cc(-n3c4ccccc4c4ccccc43)c2C#N)c1]

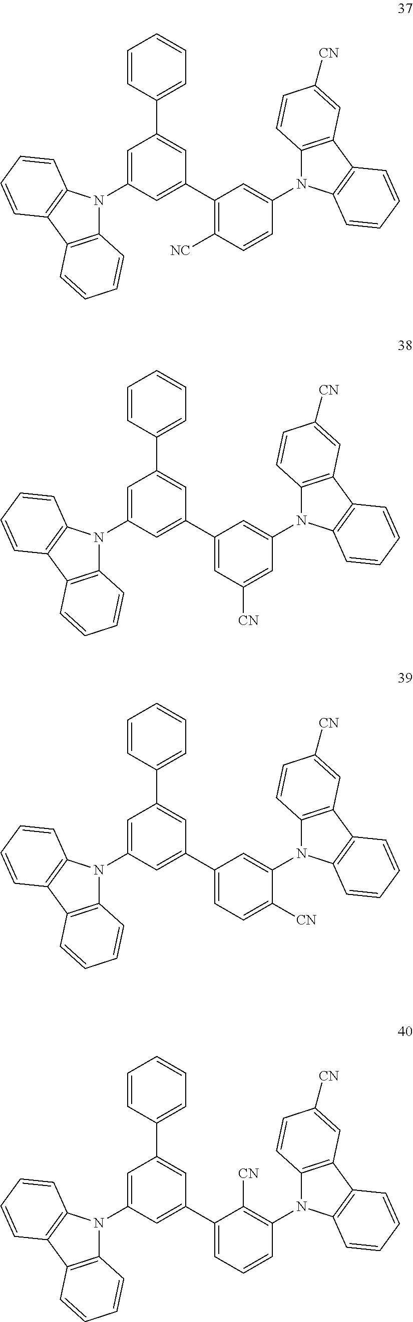 [SMILES: N#Cc1ccc2c(c1)c1ccccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccccc3c3cc(C#N)ccc32)c1 ; N#Cc1ccc2c(c1)c1ccccc1n2-c1cc(-c2cc(-c3ccccc3)cc(-n3c4ccccc4c4ccccc43)c2)ccc1C#N ; N#Cc1ccc2c(c1)c1ccccc1n2-c1cccc(-c2cc(-c3ccccc3)cc(-n3c4ccccc4c4ccccc43)c2)c1C#N]

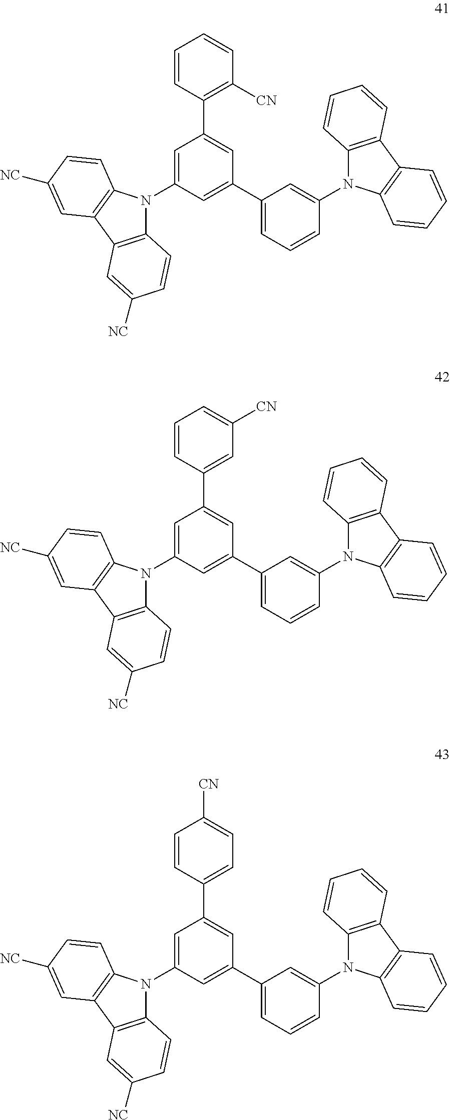 [SMILES: N#Cc1ccc2c(c1)c1cc(C#N)ccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccc(C#N)cc4c4cc(C#N)ccc43)c2)c1 ; N#Cc1ccc(-c2cc(-c3cccc(-n4c5ccccc5c5ccccc54)c3)cc(-n3c4ccc(C#N)cc4c4cc(C#N)ccc43)c2)cc1]

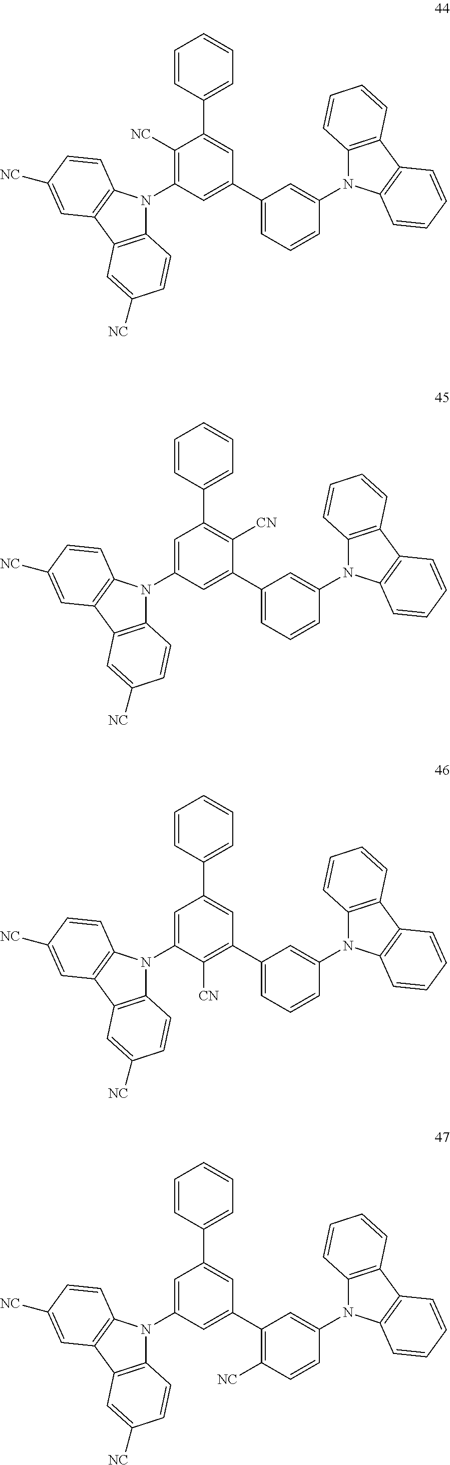 [SMILES: N#Cc1ccc2c(c1)c1cc(C#N)ccc1n2-c1cc(-c2cccc(-n3c4ccccc4c4ccccc43)c2)cc(-c2ccccc2)c1C#N ; N#Cc1ccc2c(c1)c1cc(C#N)ccc1n2-c1cc(-c2ccccc2)c(C#N)c(-c2cccc(-n3c4ccccc4c4ccccc43)c2)c1 ; N#Cc1ccc2c(c1)c1cc(C#N)ccc1n2-c1cc(-c2ccccc2)cc(-c2cccc(-n3c4ccccc4c4ccccc43)c2)c1C#N ; N#Cc1ccc2c(c1)c1cc(C#N)ccc1n2-c1cc(-c2ccccc2)cc(-c2cc(-n3c4ccccc4c4ccccc43)ccc2C#N)c1]

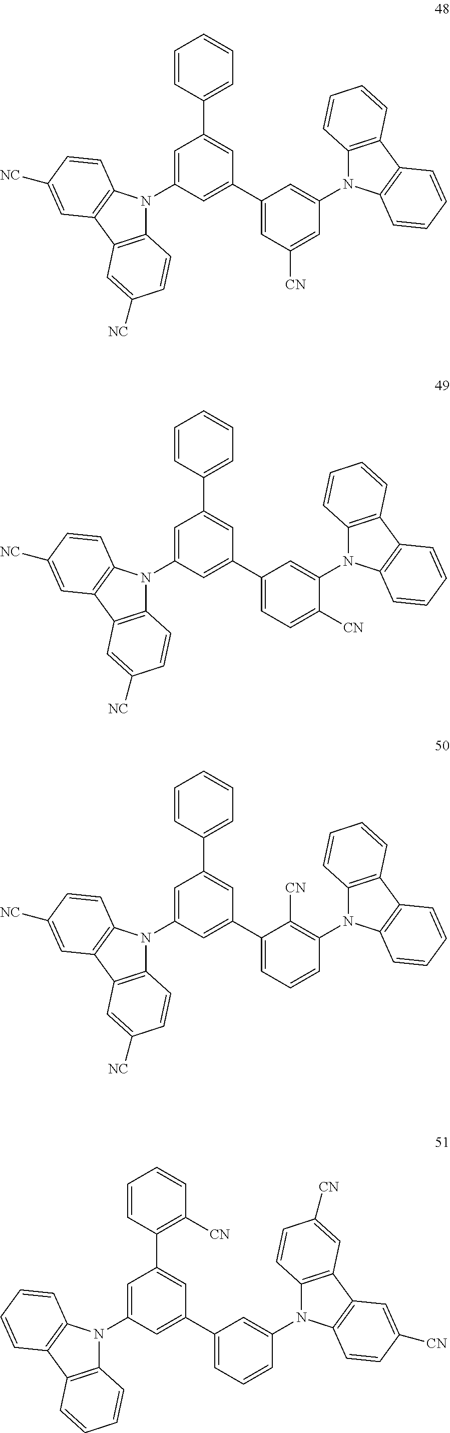 [SMILES: N#Cc1cc(-c2cc(-c3ccccc3)cc(-n3c4ccc(C#N)cc4c4cc(C#N)ccc43)c2)cc(-n2c3ccccc3c3ccccc32)c1 ; N#Cc1ccc2c(c1)c1cc(C#N)ccc1n2-c1cc(-c2ccccc2)cc(-c2ccc(C#N)c(-n3c4ccccc4c4ccccc43)c2)c1 ; N#Cc1ccc2c(c1)c1cc(C#N)ccc1n2-c1cc(-c2ccccc2)cc(-c2cccc(-n3c4ccccc4c4ccccc43)c2C#N)c1 ; N#Cc1ccc2c(c1)c1cc(C#N)ccc1n2-c1cccc(-c2cc(-c3ccccc3C#N)cc(-n3c4ccccc4c4ccccc43)c2)c1]

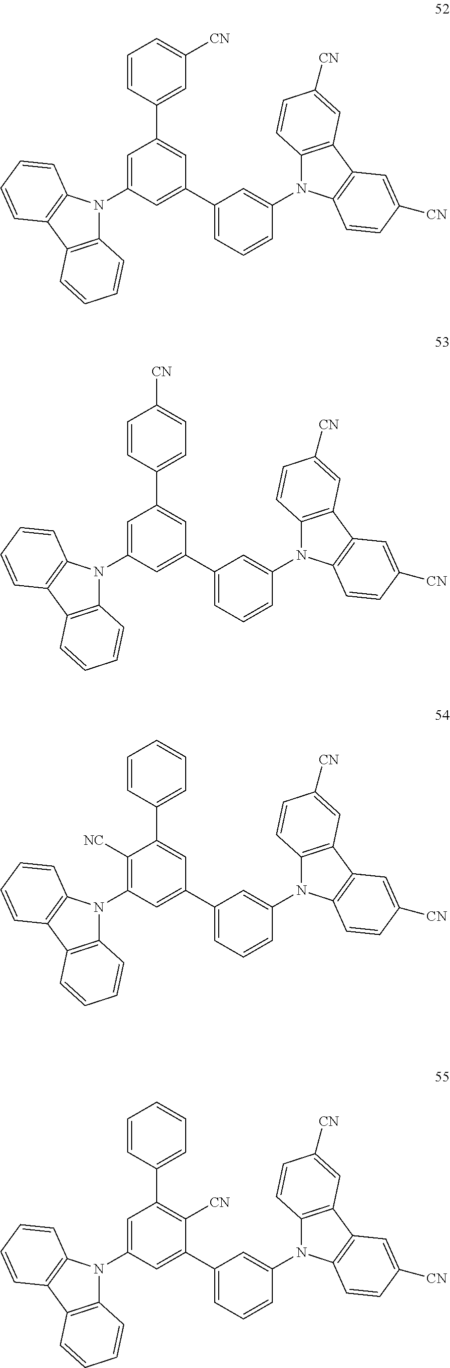 [SMILES: N#Cc1cccc(-c2cc(-c3cccc(-n4c5ccc(C#N)cc5c5cc(C#N)ccc54)c3)cc(-n3c4ccccc4c4ccccc43)c2)c1 ; N#Cc1ccc(-c2cc(-c3cccc(-n4c5ccc(C#N)cc5c5cc(C#N)ccc54)c3)cc(-n3c4ccccc4c4ccccc43)c2)cc1 ; N#Cc1ccc2c(c1)c1cc(C#N)ccc1n2-c1cccc(-c2cc(-c3ccccc3)c(C#N)c(-n3c4ccccc4c4ccccc43)c2)c1 ; N#Cc1ccc2c(c1)c1cc(C#N)ccc1n2-c1cccc(-c2cc(-n3c4ccccc4c4ccccc43)cc(-c3ccccc3)c2C#N)c1]

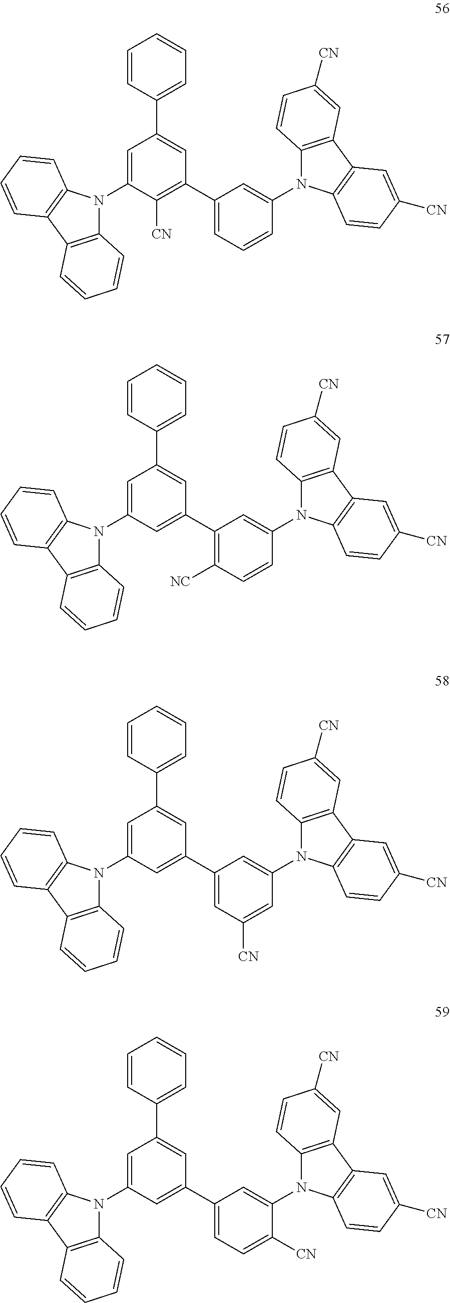 [SMILES: N#Cc1ccc2c(c1)c1cc(C#N)ccc1n2-c1cccc(-c2cc(-c3ccccc3)cc(-n3c4ccccc4c4ccccc43)c2C#N)c1 ; N#Cc1ccc2c(c1)c1cc(C#N)ccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccc(C#N)cc3c3cc(C#N)ccc32)c1 ; N#Cc1ccc2c(c1)c1cc(C#N)ccc1n2-c1cc(-c2cc(-c3ccccc3)cc(-n3c4ccccc4c4ccccc43)c2)ccc1C#N]

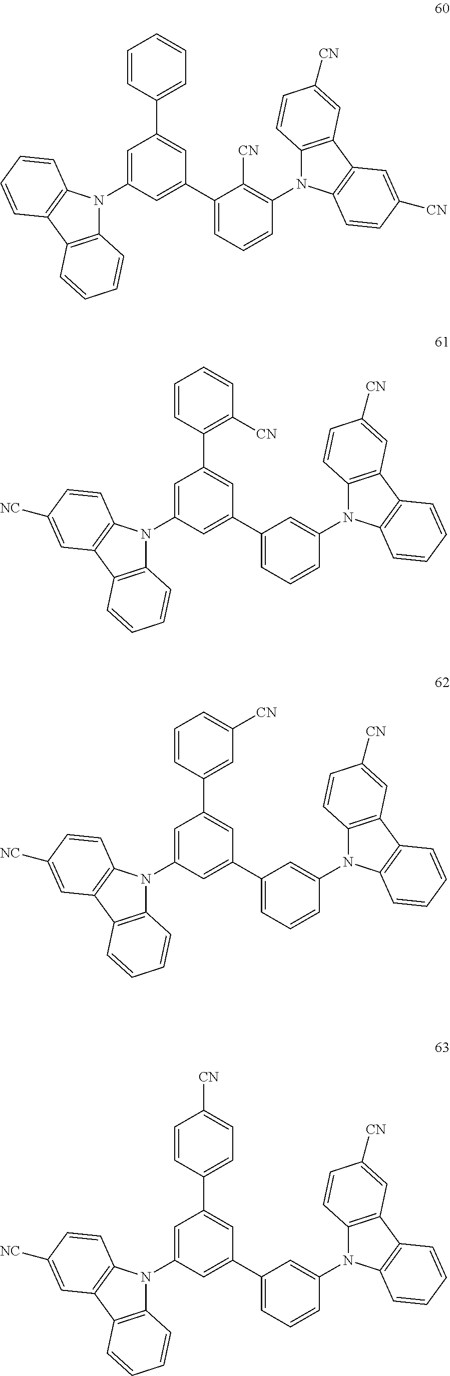 [SMILES: N#Cc1ccc2c(c1)c1cc(C#N)ccc1n2-c1cccc(-c2cc(-c3ccccc3)cc(-n3c4ccccc4c4ccccc43)c2)c1C#N ; N#Cc1ccc2c(c1)c1ccccc1n2-c1cccc(-c2cc(-c3ccccc3C#N)cc(-n3c4ccccc4c4cc(C#N)ccc43)c2)c1 ; N#Cc1cccc(-c2cc(-c3cccc(-n4c5ccccc5c5cc(C#N)ccc54)c3)cc(-n3c4ccccc4c4cc(C#N)ccc43)c2)c1 ; N#Cc1ccc(-c2cc(-c3cccc(-n4c5ccccc5c5cc(C#N)ccc54)c3)cc(-n3c4ccccc4c4cc(C#N)ccc43)c2)cc1]

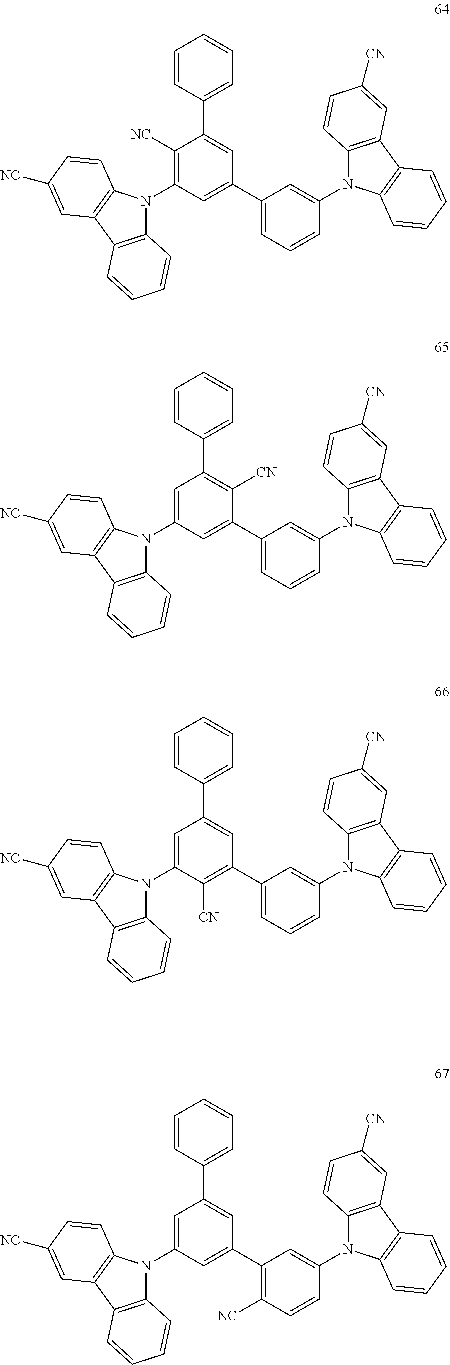 [SMILES: N#Cc1ccc2c(c1)c1ccccc1n2-c1cccc(-c2cc(-c3ccccc3)c(C#N)c(-n3c4ccccc4c4cc(C#N)ccc43)c2)c1 ; N#Cc1ccc2c(c1)c1ccccc1n2-c1cccc(-c2cc(-n3c4ccccc4c4cc(C#N)ccc43)cc(-c3ccccc3)c2C#N)c1 ; N#Cc1ccc2c(c1)c1ccccc1n2-c1cccc(-c2cc(-c3ccccc3)cc(-n3c4ccccc4c4cc(C#N)ccc43)c2C#N)c1 ; N#Cc1ccc2c(c1)c1ccccc1n2-c1cc(-c2ccccc2)cc(-c2cc(-n3c4ccccc4c4cc(C#N)ccc43)ccc2C#N)c1]

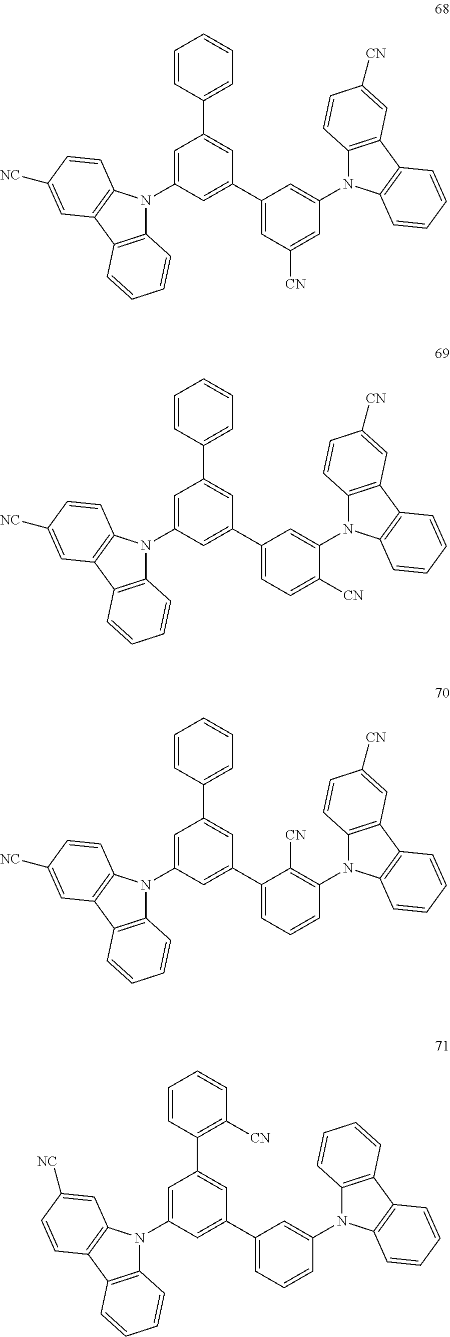 [SMILES: N#Cc1cc(-c2cc(-c3ccccc3)cc(-n3c4ccccc4c4cc(C#N)ccc43)c2)cc(-n2c3ccccc3c3cc(C#N)ccc32)c1 ; N#Cc1ccc2c(c1)c1ccccc1n2-c1cc(-c2ccccc2)cc(-c2ccc(C#N)c(-n3c4ccccc4c4cc(C#N)ccc43)c2)c1 ; N#Cc1ccc2c(c1)c1ccccc1n2-c1cc(-c2ccccc2)cc(-c2cccc(-n3c4ccccc4c4cc(C#N)ccc43)c2C#N)c1 ; N#Cc1ccc2c3ccccc3n(-c3cc(-c4cccc(-n5c6ccccc6c6ccccc65)c4)cc(-c4ccccc4C#N)c3)c2c1]

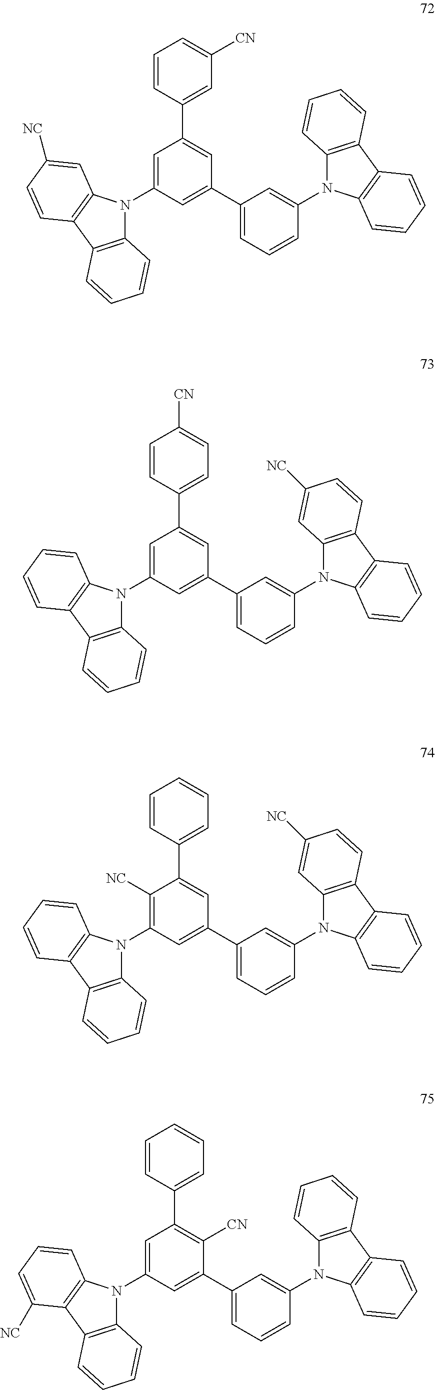 [SMILES: N#Cc1cccc(-c2cc(-c3cccc(-n4c5ccccc5c5ccccc54)c3)cc(-n3c4ccccc4c4ccc(C#N)cc43)c2)c1 ; N#Cc1ccc(-c2cc(-c3cccc(-n4c5ccccc5c5ccc(C#N)cc54)c3)cc(-n3c4ccccc4c4ccccc43)c2)cc1 ; N#Cc1ccc2c3ccccc3n(-c3cccc(-c4cc(-c5ccccc5)c(C#N)c(-n5c6ccccc6c6ccccc65)c4)c3)c2c1 ; N#Cc1c(-c2ccccc2)cc(-n2c3ccccc3c3c(C#N)cccc32)cc1-c1cccc(-n2c3ccccc3c3ccccc32)c1]

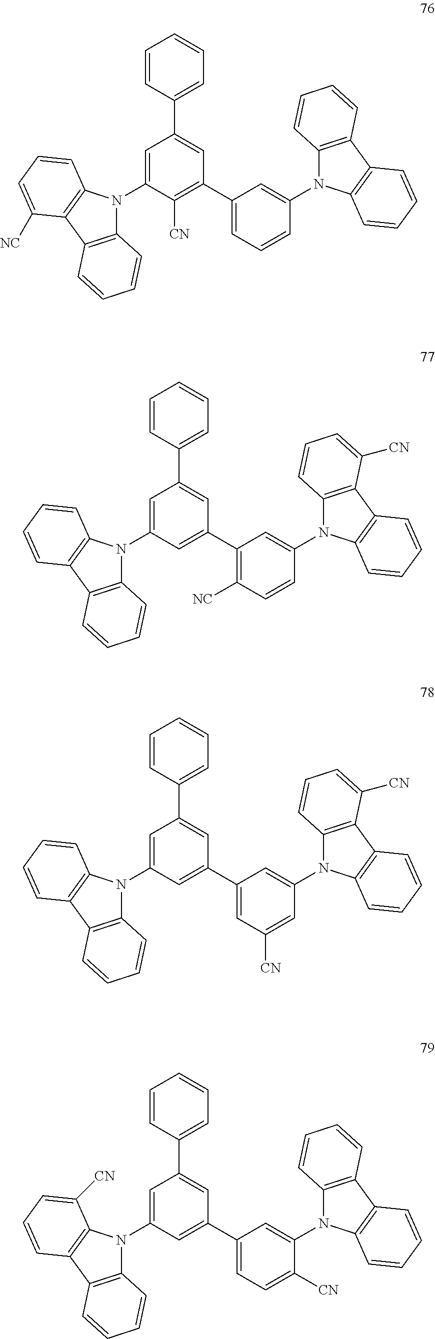 [SMILES: N#Cc1c(-c2cccc(-n3c4ccccc4c4ccccc43)c2)cc(-c2ccccc2)cc1-n1c2ccccc2c2c(C#N)cccc21 ; N#Cc1ccc(-n2c3ccccc3c3c(C#N)cccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3c(C#N)cccc32)c1 ; N#Cc1ccc(-c2cc(-c3ccccc3)cc(-n3c4ccccc4c4cccc(C#N)c43)c2)cc1-n1c2ccccc2c2ccccc21]

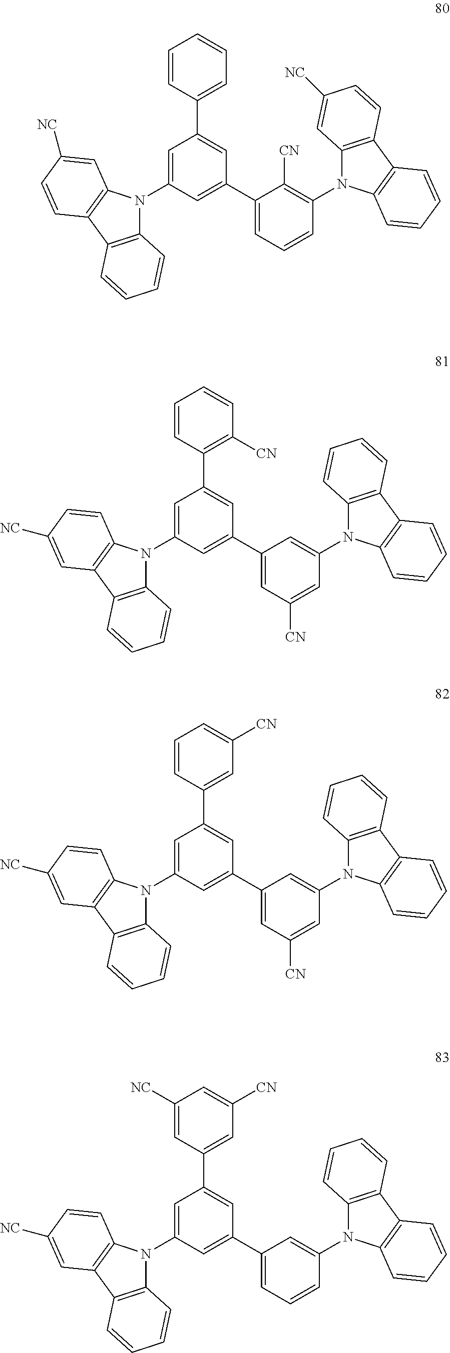 [SMILES: N#Cc1ccc2c3ccccc3n(-c3cc(-c4ccccc4)cc(-c4cccc(-n5c6ccccc6c6ccc(C#N)cc65)c4C#N)c3)c2c1 ; N#Cc1cc(-c2cc(-c3ccccc3C#N)cc(-n3c4ccccc4c4cc(C#N)ccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4cc(C#N)ccc43)c2)c1 ; N#Cc1cc(C#N)cc(-c2cc(-c3cccc(-n4c5ccccc5c5ccccc54)c3)cc(-n3c4ccccc4c4cc(C#N)ccc43)c2)c1]

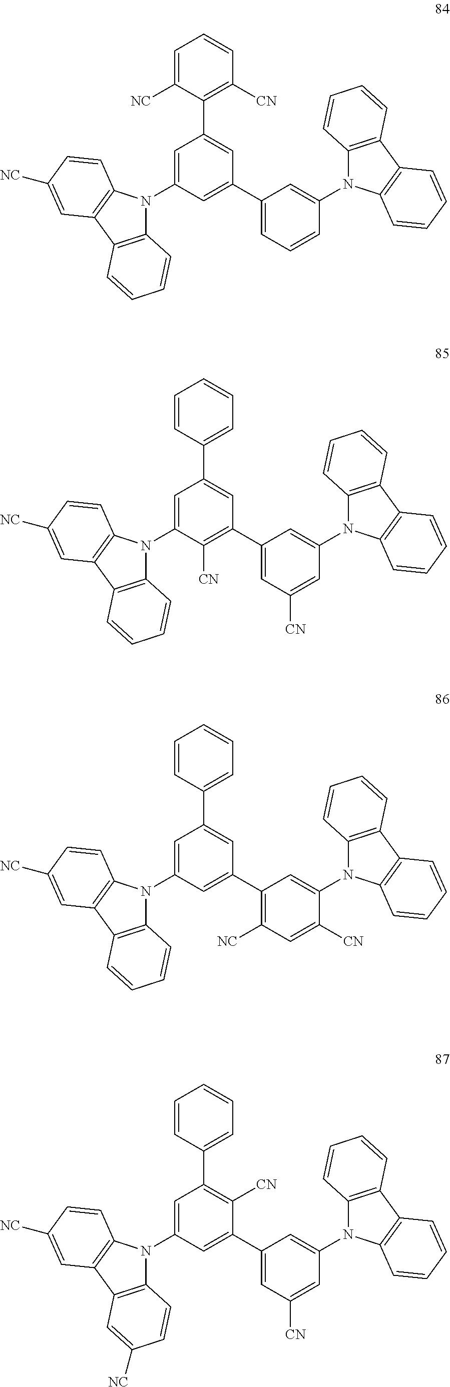 [SMILES: N#Cc1ccc2c(c1)c1ccccc1n2-c1cc(-c2cccc(-n3c4ccccc4c4ccccc43)c2)cc(-c2c(C#N)cccc2C#N)c1 ; N#Cc1cc(-c2cc(-c3ccccc3)cc(-n3c4ccccc4c4cc(C#N)ccc43)c2C#N)cc(-n2c3ccccc3c3ccccc32)c1 ; N#Cc1ccc2c(c1)c1ccccc1n2-c1cc(-c2ccccc2)cc(-c2cc(-n3c4ccccc4c4ccccc43)c(C#N)cc2C#N)c1 ; N#Cc1cc(-c2cc(-n3c4ccc(C#N)cc4c4cc(C#N)ccc43)cc(-c3ccccc3)c2C#N)cc(-n2c3ccccc3c3ccccc32)c1]

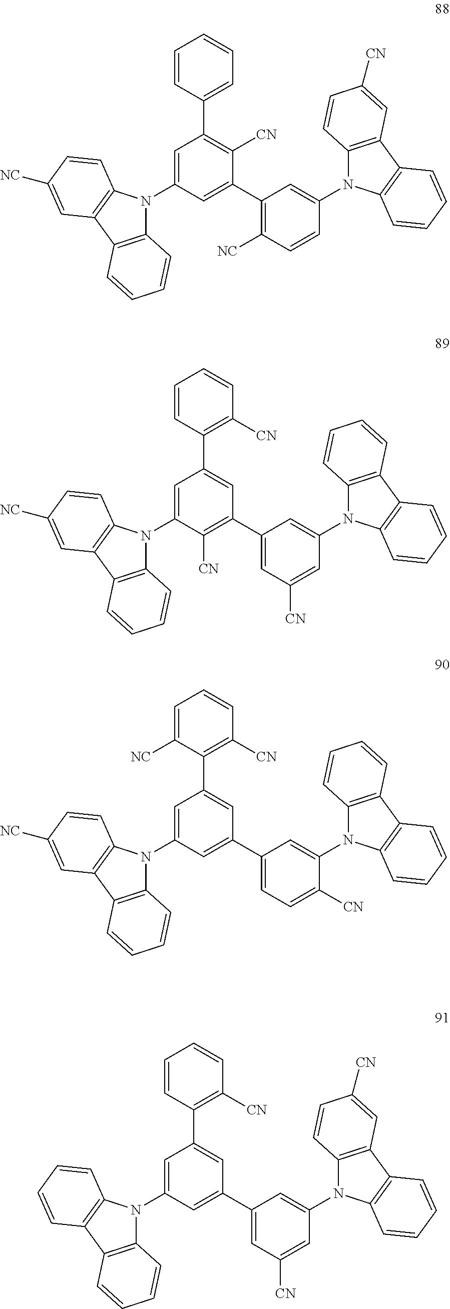 [SMILES: N#Cc1ccc2c(c1)c1ccccc1n2-c1ccc(C#N)c(-c2cc(-n3c4ccccc4c4cc(C#N)ccc43)cc(-c3ccccc3)c2C#N)c1 ; N#Cc1cc(-c2cc(-c3ccccc3C#N)cc(-n3c4ccccc4c4cc(C#N)ccc43)c2C#N)cc(-n2c3ccccc3c3ccccc32)c1 ; N#Cc1ccc2c(c1)c1ccccc1n2-c1cc(-c2ccc(C#N)c(-n3c4ccccc4c4ccccc43)c2)cc(-c2c(C#N)cccc2C#N)c1 ; N#Cc1cc(-c2cc(-c3ccccc3C#N)cc(-n3c4ccccc4c4ccccc43)c2)cc(-n2c3ccccc3c3cc(C#N)ccc32)c1]

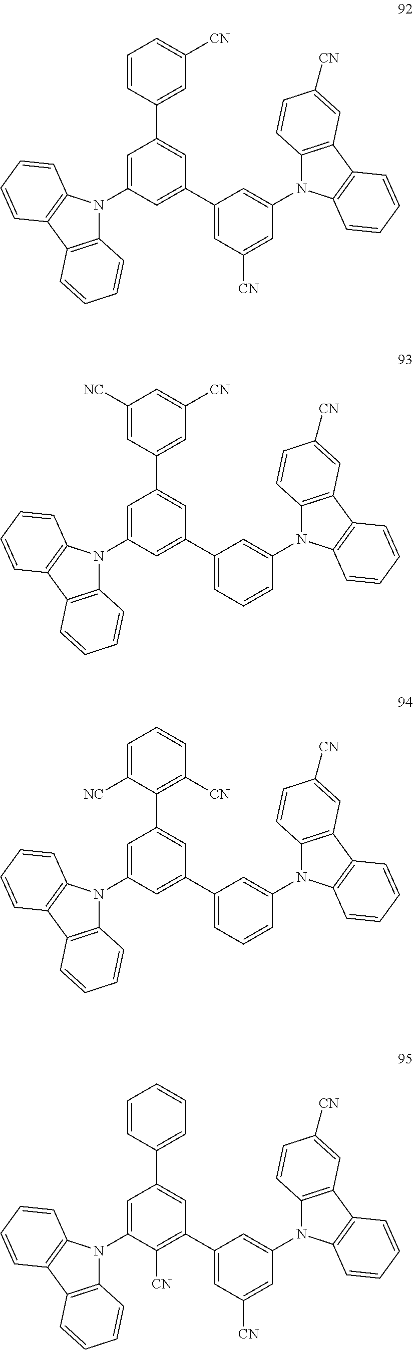 [SMILES: N#Cc1cccc(-c2cc(-c3cc(C#N)cc(-n4c5ccccc5c5cc(C#N)ccc54)c3)cc(-n3c4ccccc4c4ccccc43)c2)c1 ; N#Cc1cc(C#N)cc(-c2cc(-c3cccc(-n4c5ccccc5c5cc(C#N)ccc54)c3)cc(-n3c4ccccc4c4ccccc43)c2)c1 ; N#Cc1ccc2c(c1)c1ccccc1n2-c1cccc(-c2cc(-c3c(C#N)cccc3C#N)cc(-n3c4ccccc4c4ccccc43)c2)c1 ; N#Cc1cc(-c2cc(-c3ccccc3)cc(-n3c4ccccc4c4ccccc43)c2C#N)cc(-n2c3ccccc3c3cc(C#N)ccc32)c1]

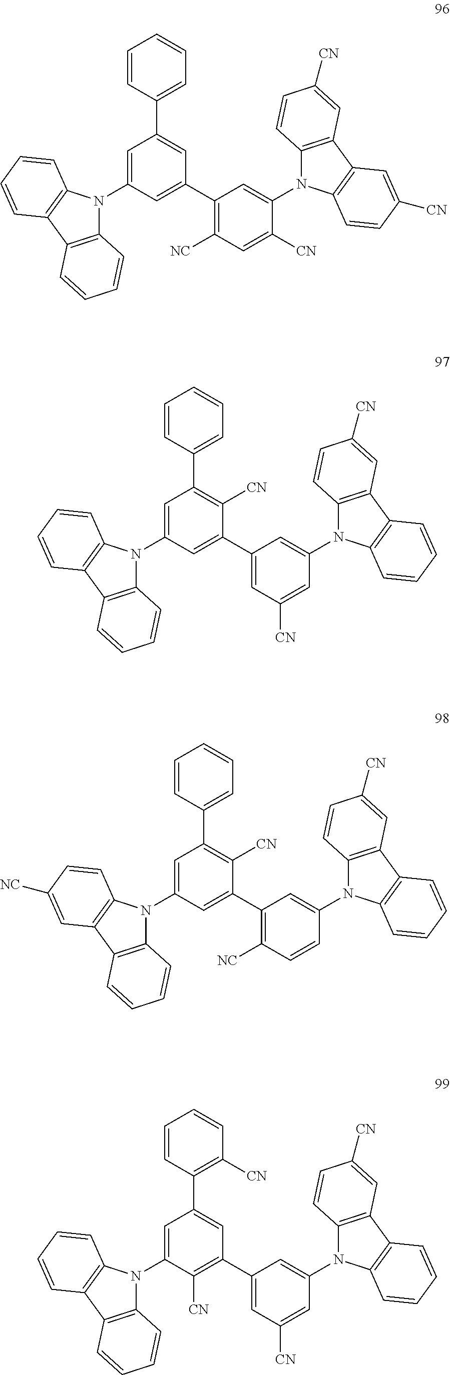 [SMILES: N#Cc1ccc2c(c1)c1cc(C#N)ccc1n2-c1cc(-c2cc(-c3ccccc3)cc(-n3c4ccccc4c4ccccc43)c2)c(C#N)cc1C#N ; N#Cc1cc(-c2cc(-n3c4ccccc4c4ccccc43)cc(-c3ccccc3)c2C#N)cc(-n2c3ccccc3c3cc(C#N)ccc32)c1 ; N#Cc1ccc2c(c1)c1ccccc1n2-c1ccc(C#N)c(-c2cc(-n3c4ccccc4c4cc(C#N)ccc43)cc(-c3ccccc3)c2C#N)c1 ; N#Cc1cc(-c2cc(-c3ccccc3C#N)cc(-n3c4ccccc4c4ccccc43)c2C#N)cc(-n2c3ccccc3c3cc(C#N)ccc32)c1]

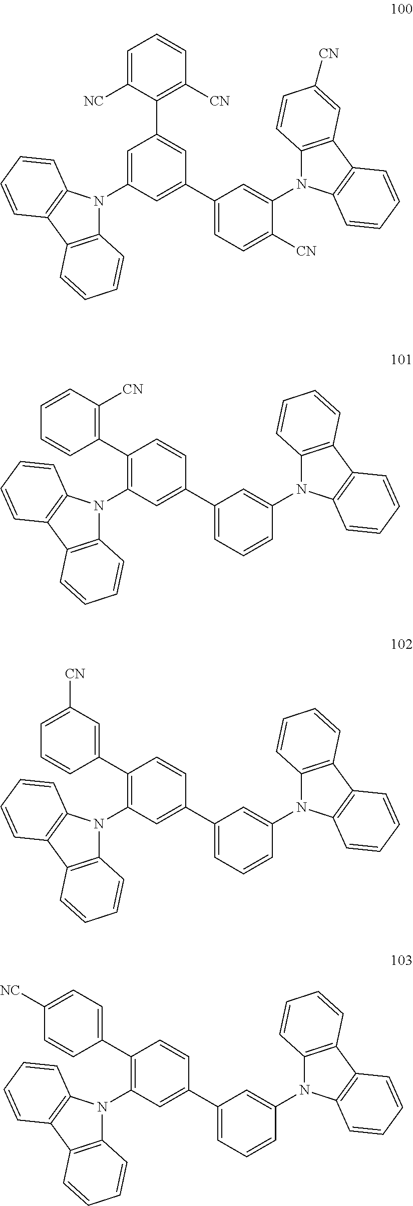 [SMILES: N#Cc1ccc2c(c1)c1ccccc1n2-c1cc(-c2cc(-c3c(C#N)cccc3C#N)cc(-n3c4ccccc4c4ccccc43)c2)ccc1C#N ; N#Cc1ccccc1-c1ccc(-c2cccc(-n3c4ccccc4c4ccccc43)c2)cc1-n1c2ccccc2c2ccccc21 ; N#Cc1cccc(-c2ccc(-c3cccc(-n4c5ccccc5c5ccccc54)c3)cc2-n2c3ccccc3c3ccccc32)c1 ; N#Cc1ccc(-c2ccc(-c3cccc(-n4c5ccccc5c5ccccc54)c3)cc2-n2c3ccccc3c3ccccc32)cc1]

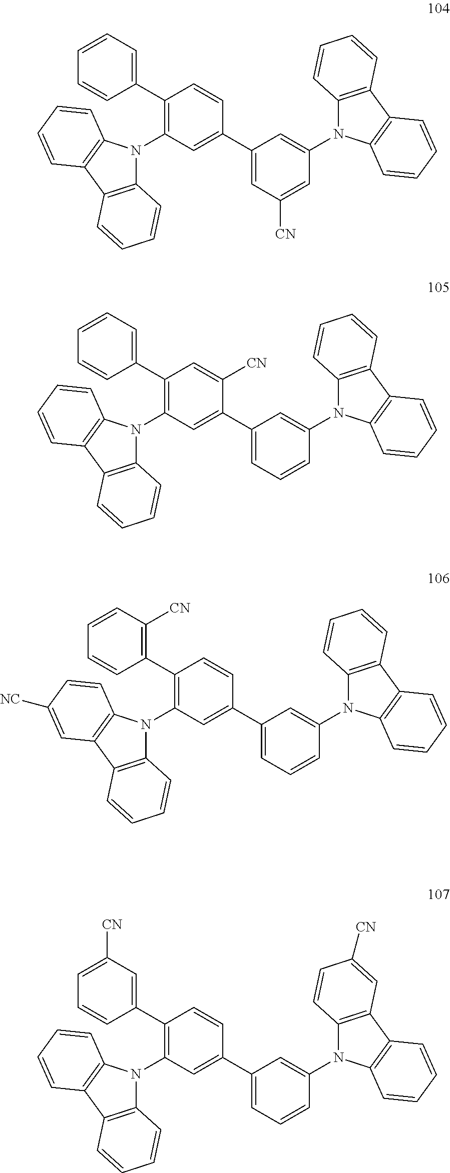 [SMILES: N#Cc1cc(-c2ccc(-c3ccccc3)c(-n3c4ccccc4c4ccccc43)c2)cc(-n2c3ccccc3c3ccccc32)c1 ; N#Cc1cc(-c2ccccc2)c(-n2c3ccccc3c3ccccc32)cc1-c1cccc(-n2c3ccccc3c3ccccc32)c1 ; N#Cc1ccc2c(c1)c1ccccc1n2-c1cc(-c2cccc(-n3c4ccccc4c4ccccc43)c2)ccc1-c1ccccc1C#N ; N#Cc1cccc(-c2ccc(-c3cccc(-n4c5ccccc5c5cc(C#N)ccc54)c3)cc2-n2c3ccccc3c3ccccc32)c1]

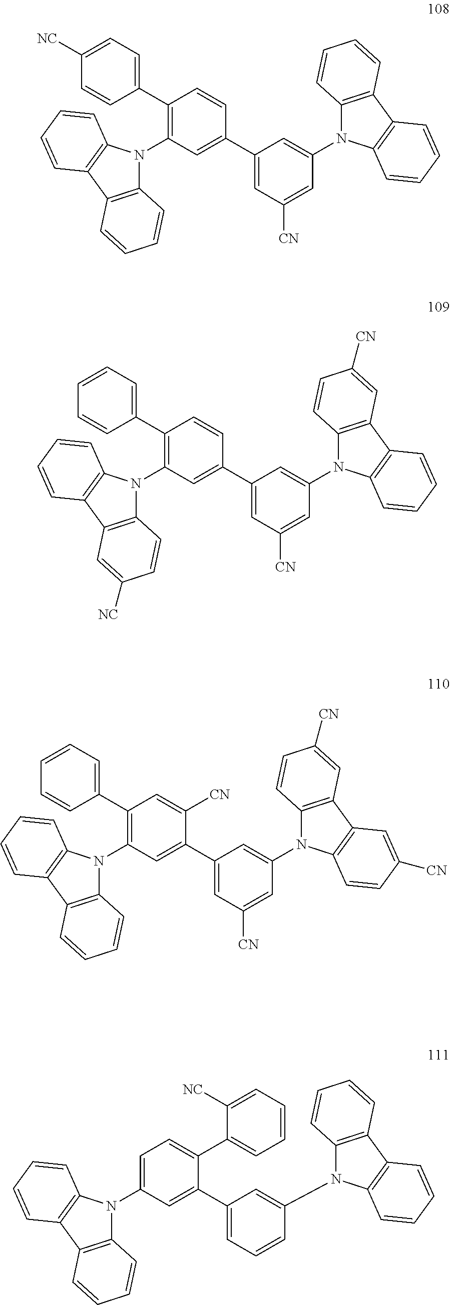 [SMILES: N#Cc1ccc(-c2ccc(-c3cc(C#N)cc(-n4c5ccccc5c5ccccc54)c3)cc2-n2c3ccccc3c3ccccc32)cc1 ; N#Cc1cc(-c2ccc(-c3ccccc3)c(-n3c4ccccc4c4cc(C#N)ccc43)c2)cc(-n2c3ccccc3c3cc(C#N)ccc32)c1 ; N#Cc1cc(-c2cc(-n3c4ccccc4c4ccccc43)c(-c3ccccc3)cc2C#N)cc(-n2c3ccc(C#N)cc3c3cc(C#N)ccc32)c1 ; N#Cc1ccccc1-c1ccc(-n2c3ccccc3c3ccccc32)cc1-c1cccc(-n2c3ccccc3c3ccccc32)c1]

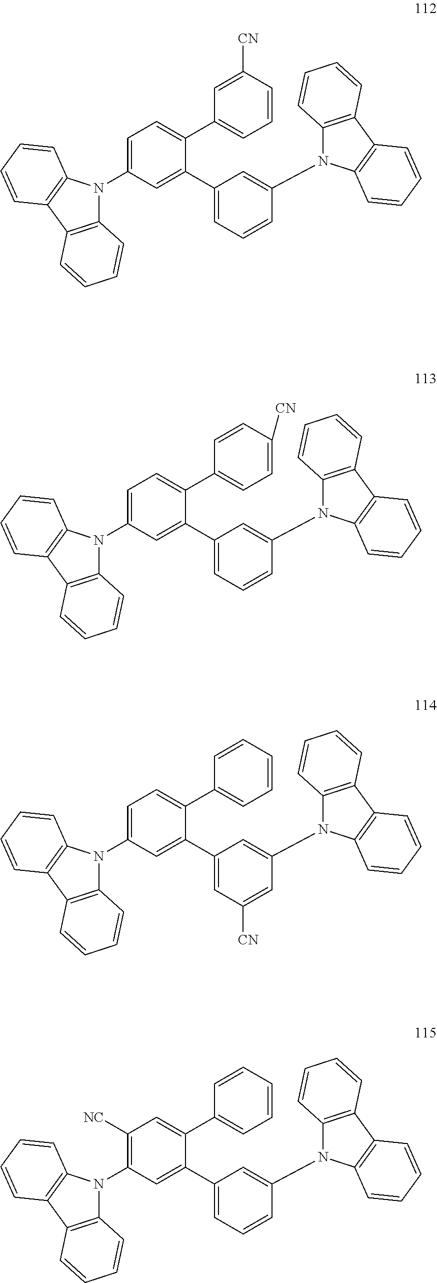 [SMILES: N#Cc1cccc(-c2ccc(-n3c4ccccc4c4ccccc43)cc2-c2cccc(-n3c4ccccc4c4ccccc43)c2)c1 ; N#Cc1ccc(-c2ccc(-n3c4ccccc4c4ccccc43)cc2-c2cccc(-n3c4ccccc4c4ccccc43)c2)cc1 ; N#Cc1cc(-c2cc(-n3c4ccccc4c4ccccc43)ccc2-c2ccccc2)cc(-n2c3ccccc3c3ccccc32)c1 ; N#Cc1cc(-c2ccccc2)c(-c2cccc(-n3c4ccccc4c4ccccc43)c2)cc1-n1c2ccccc2c2ccccc21]

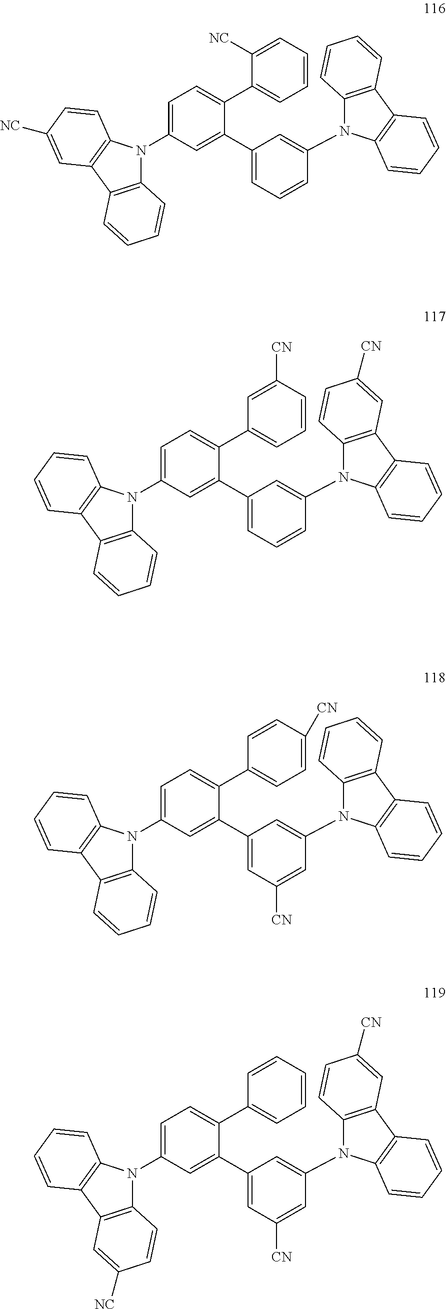 [SMILES: N#Cc1ccc2c(c1)c1ccccc1n2-c1ccc(-c2ccccc2C#N)c(-c2cccc(-n3c4ccccc4c4ccccc43)c2)c1 ; N#Cc1cccc(-c2ccc(-n3c4ccccc4c4ccccc43)cc2-c2cccc(-n3c4ccccc4c4cc(C#N)ccc43)c2)c1 ; N#Cc1ccc(-c2ccc(-n3c4ccccc4c4ccccc43)cc2-c2cc(C#N)cc(-n3c4ccccc4c4ccccc43)c2)cc1 ; N#Cc1cc(-c2cc(-n3c4ccccc4c4cc(C#N)ccc43)ccc2-c2ccccc2)cc(-n2c3ccccc3c3cc(C#N)ccc32)c1]

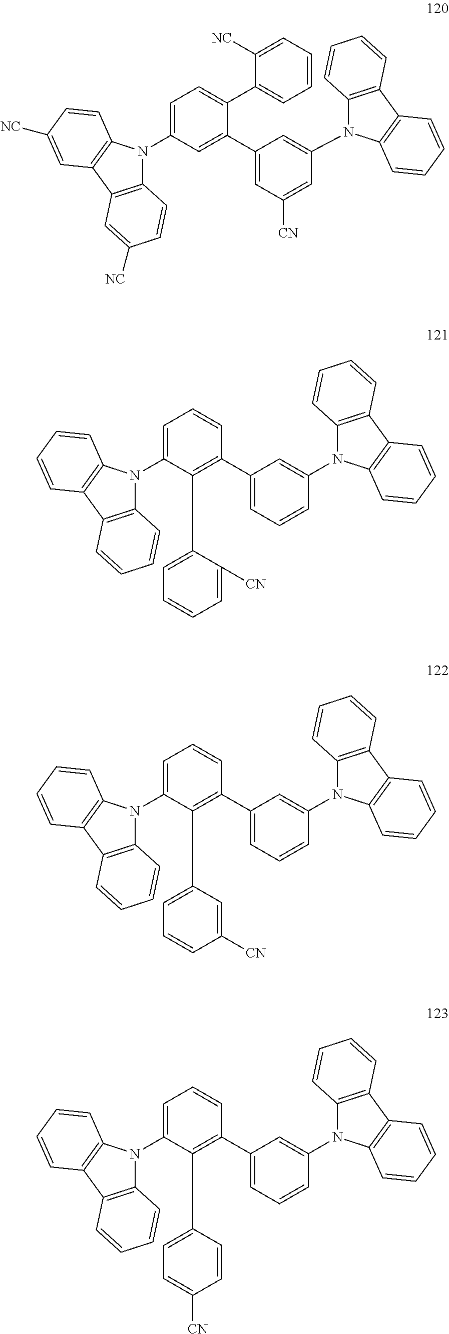 [SMILES: N#Cc1cc(-c2cc(-n3c4ccc(C#N)cc4c4cc(C#N)ccc43)ccc2-c2ccccc2C#N)cc(-n2c3ccccc3c3ccccc32)c1 ; N#Cc1ccccc1-c1c(-c2cccc(-n3c4ccccc4c4ccccc43)c2)cccc1-n1c2ccccc2c2ccccc21 ; N#Cc1cccc(-c2c(-c3cccc(-n4c5ccccc5c5ccccc54)c3)cccc2-n2c3ccccc3c3ccccc32)c1 ; N#Cc1ccc(-c2c(-c3cccc(-n4c5ccccc5c5ccccc54)c3)cccc2-n2c3ccccc3c3ccccc32)cc1]

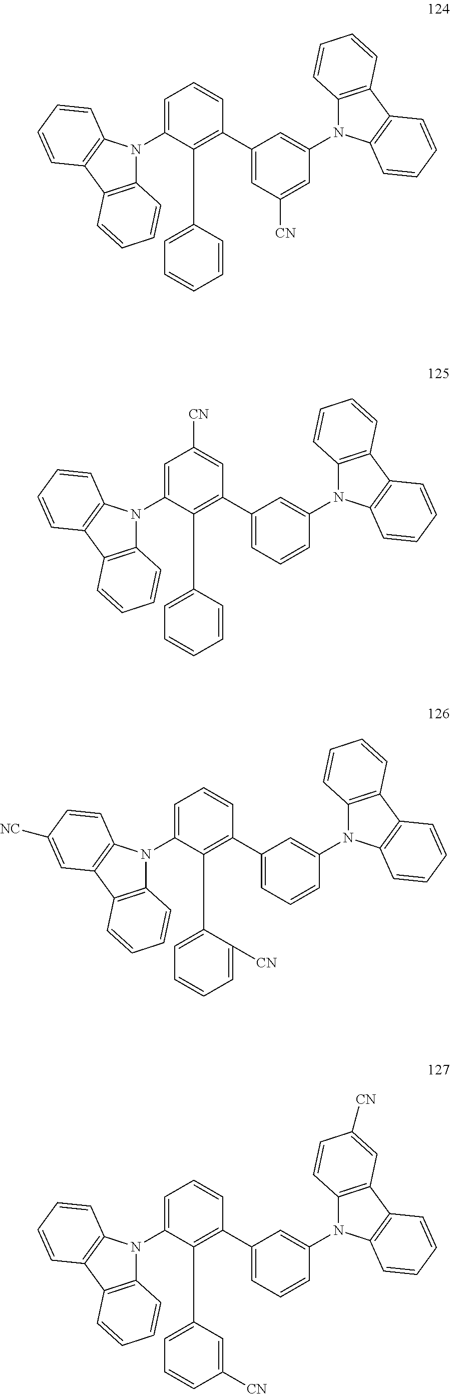 [SMILES: N#Cc1cc(-c2cccc(-n3c4ccccc4c4ccccc43)c2-c2ccccc2)cc(-n2c3ccccc3c3ccccc32)c1 ; N#Cc1cc(-c2cccc(-n3c4ccccc4c4ccccc43)c2)c(-c2ccccc2)c(-n2c3ccccc3c3ccccc32)c1 ; N#Cc1ccc2c(c1)c1ccccc1n2-c1cccc(-c2cccc(-n3c4ccccc4c4ccccc43)c2)c1-c1ccccc1C#N ; N#Cc1cccc(-c2c(-c3cccc(-n4c5ccccc5c5cc(C#N)ccc54)c3)cccc2-n2c3ccccc3c3ccccc32)c1]

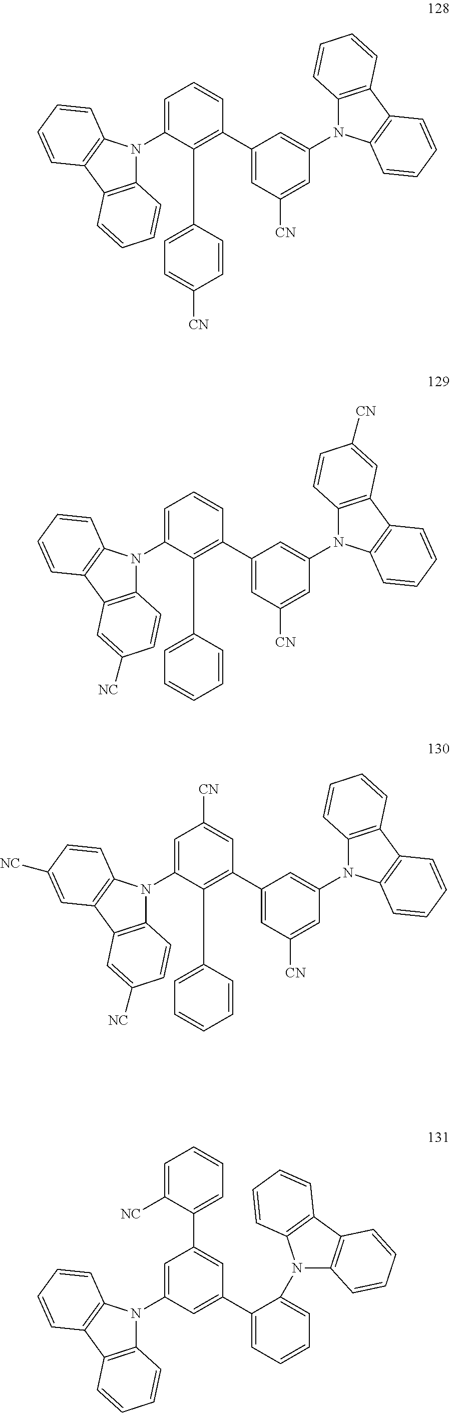 [SMILES: N#Cc1ccc(-c2c(-c3cc(C#N)cc(-n4c5ccccc5c5ccccc54)c3)cccc2-n2c3ccccc3c3ccccc32)cc1 ; N#Cc1cc(-c2cccc(-n3c4ccccc4c4cc(C#N)ccc43)c2-c2ccccc2)cc(-n2c3ccccc3c3cc(C#N)ccc32)c1 ; N#Cc1cc(-c2cc(C#N)cc(-n3c4ccc(C#N)cc4c4cc(C#N)ccc43)c2-c2ccccc2)cc(-n2c3ccccc3c3ccccc32)c1 ; N#Cc1ccccc1-c1cc(-c2ccccc2-n2c3ccccc3c3ccccc32)cc(-n2c3ccccc3c3ccccc32)c1]

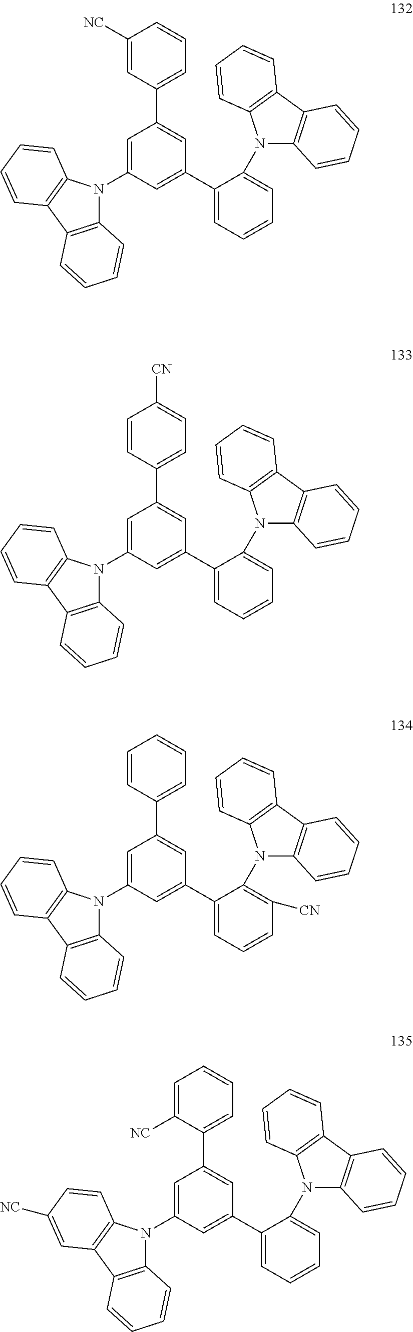 [SMILES: N#Cc1cccc(-c2cc(-c3ccccc3-n3c4ccccc4c4ccccc43)cc(-n3c4ccccc4c4ccccc43)c2)c1 ; N#Cc1ccc(-c2cc(-c3ccccc3-n3c4ccccc4c4ccccc43)cc(-n3c4ccccc4c4ccccc43)c2)cc1 ; N#Cc1cccc(-c2cc(-c3ccccc3)cc(-n3c4ccccc4c4ccccc43)c2)c1-n1c2ccccc2c2ccccc21 ; N#Cc1ccc2c(c1)c1ccccc1n2-c1cc(-c2ccccc2C#N)cc(-c2ccccc2-n2c3ccccc3c3ccccc32)c1]

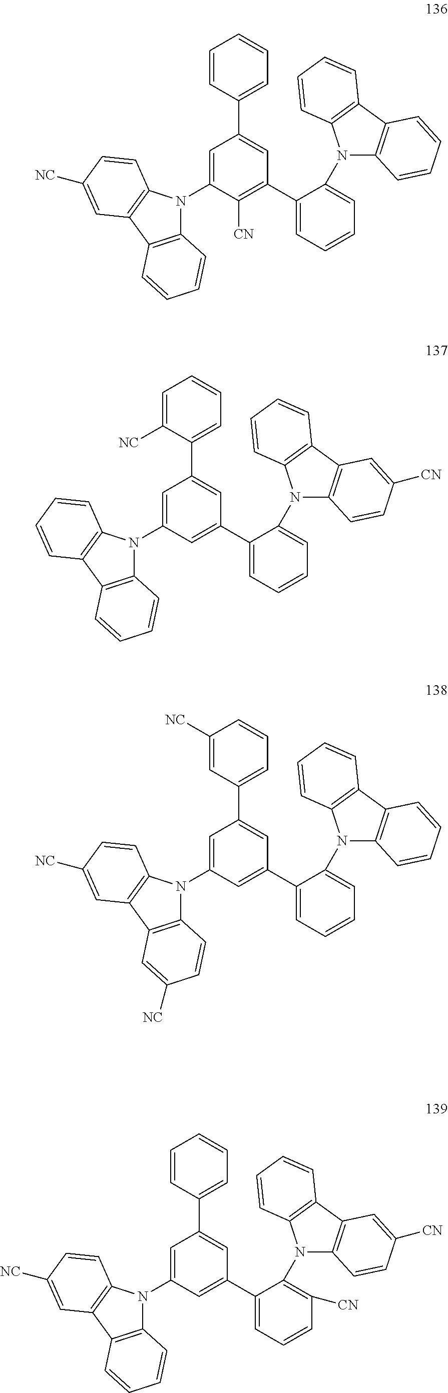 [SMILES: N#Cc1ccc2c(c1)c1ccccc1n2-c1cc(-c2ccccc2)cc(-c2ccccc2-n2c3ccccc3c3ccccc32)c1C#N ; N#Cc1ccc2c(c1)c1ccccc1n2-c1ccccc1-c1cc(-c2ccccc2C#N)cc(-n2c3ccccc3c3ccccc32)c1 ; N#Cc1cccc(-c2cc(-c3ccccc3-n3c4ccccc4c4ccccc43)cc(-n3c4ccc(C#N)cc4c4cc(C#N)ccc43)c2)c1 ; N#Cc1ccc2c(c1)c1ccccc1n2-c1cc(-c2ccccc2)cc(-c2cccc(C#N)c2-n2c3ccccc3c3cc(C#N)ccc32)c1]

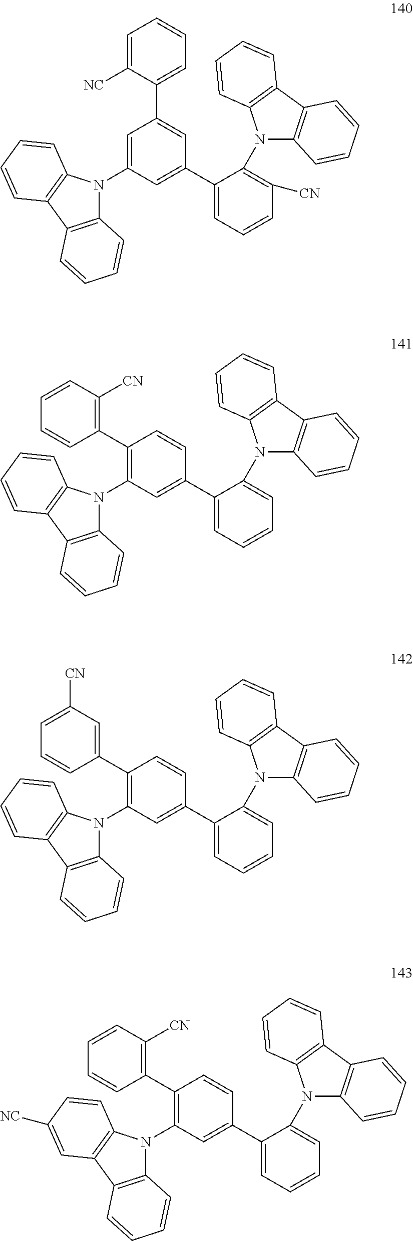 [SMILES: N#Cc1ccccc1-c1cc(-c2cccc(C#N)c2-n2c3ccccc3c3ccccc32)cc(-n2c3ccccc3c3ccccc32)c1 ; N#Cc1ccccc1-c1ccc(-c2ccccc2-n2c3ccccc3c3ccccc32)cc1-n1c2ccccc2c2ccccc21 ; N#Cc1cccc(-c2ccc(-c3ccccc3-n3c4ccccc4c4ccccc43)cc2-n2c3ccccc3c3ccccc32)c1 ; N#Cc1ccc2c(c1)c1ccccc1n2-c1cc(-c2ccccc2-n2c3ccccc3c3ccccc32)ccc1-c1ccccc1C#N]

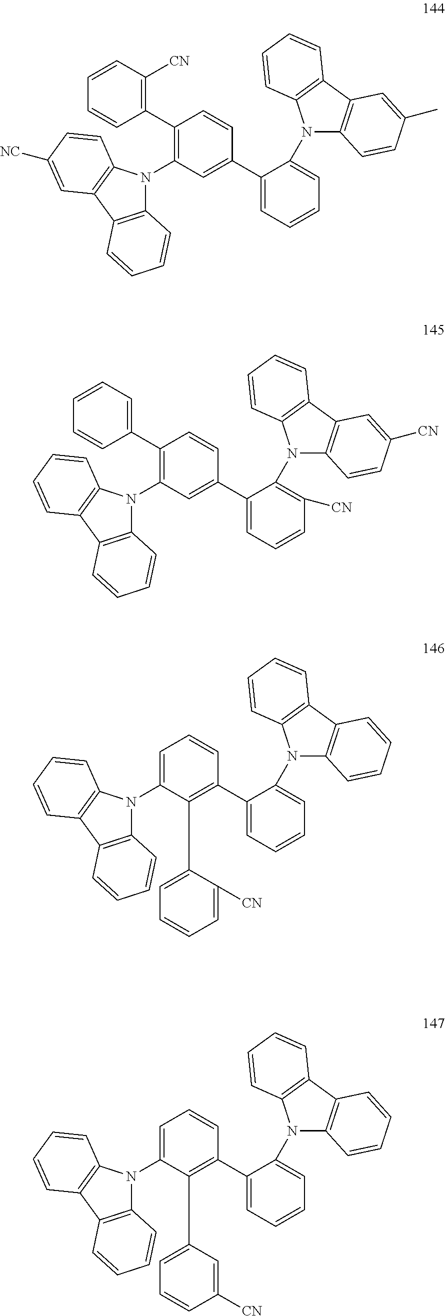 [SMILES: Cc1ccc2c(c1)c1ccccc1n2-c1ccccc1-c1ccc(-c2ccccc2C#N)c(-n2c3ccccc3c3cc(C#N)ccc32)c1 ; N#Cc1ccc2c(c1)c1ccccc1n2-c1c(C#N)cccc1-c1ccc(-c2ccccc2)c(-n2c3ccccc3c3ccccc32)c1 ; N#Cc1ccccc1-c1c(-c2ccccc2-n2c3ccccc3c3ccccc32)cccc1-n1c2ccccc2c2ccccc21 ; N#Cc1cccc(-c2c(-c3ccccc3-n3c4ccccc4c4ccccc43)cccc2-n2c3ccccc3c3ccccc32)c1]

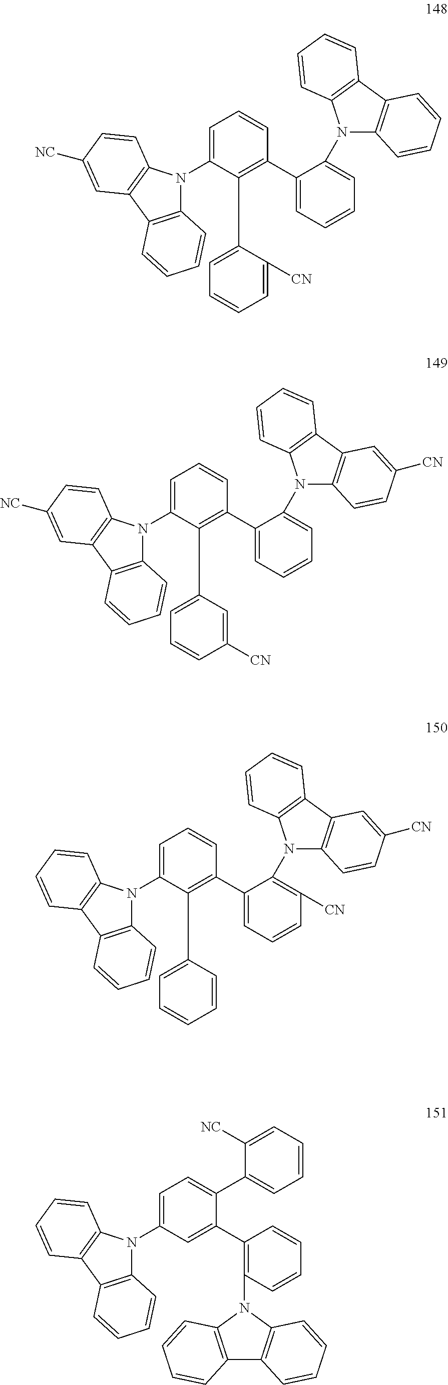 [SMILES: N#Cc1ccc2c(c1)c1ccccc1n2-c1cccc(-c2ccccc2-n2c3ccccc3c3ccccc32)c1-c1ccccc1C#N ; N#Cc1cccc(-c2c(-c3ccccc3-n3c4ccccc4c4cc(C#N)ccc43)cccc2-n2c3ccccc3c3cc(C#N)ccc32)c1 ; N#Cc1ccc2c(c1)c1ccccc1n2-c1c(C#N)cccc1-c1cccc(-n2c3ccccc3c3ccccc32)c1-c1ccccc1 ; N#Cc1ccccc1-c1ccc(-n2c3ccccc3c3ccccc32)cc1-c1ccccc1-n1c2ccccc2c2ccccc21]

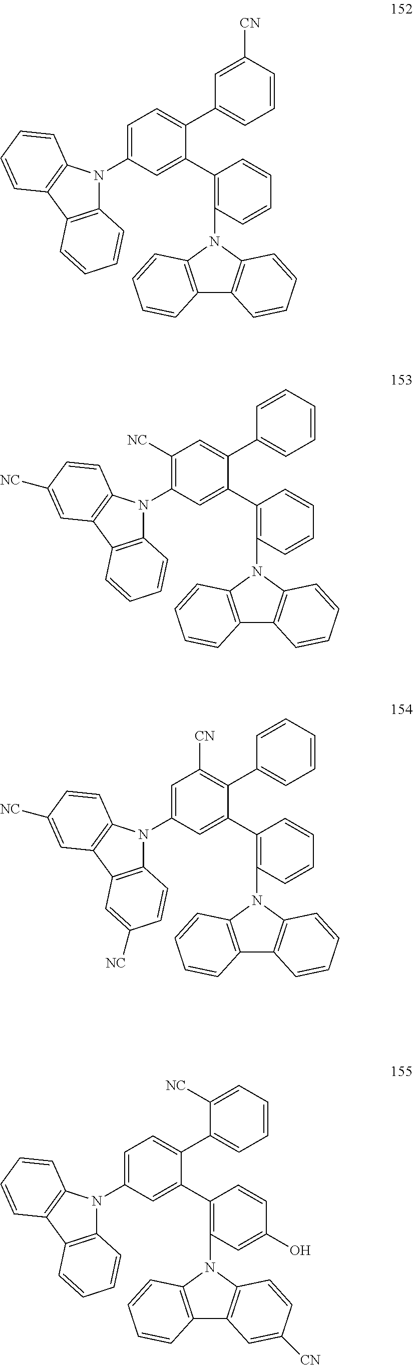 [SMILES: N#Cc1cccc(-c2ccc(-n3c4ccccc4c4ccccc43)cc2-c2ccccc2-n2c3ccccc3c3ccccc32)c1 ; N#Cc1ccc2c(c1)c1ccccc1n2-c1cc(-c2ccccc2-n2c3ccccc3c3ccccc32)c(-c2ccccc2)cc1C#N ; N#Cc1ccc2c(c1)c1cc(C#N)ccc1n2-c1cc(C#N)c(-c2ccccc2)c(-c2ccccc2-n2c3ccccc3c3ccccc32)c1 ; N#Cc1ccc2c(c1)c1ccccc1n2-c1cc(O)ccc1-c1cc(-n2c3ccccc3c3ccccc32)ccc1-c1ccccc1C#N]

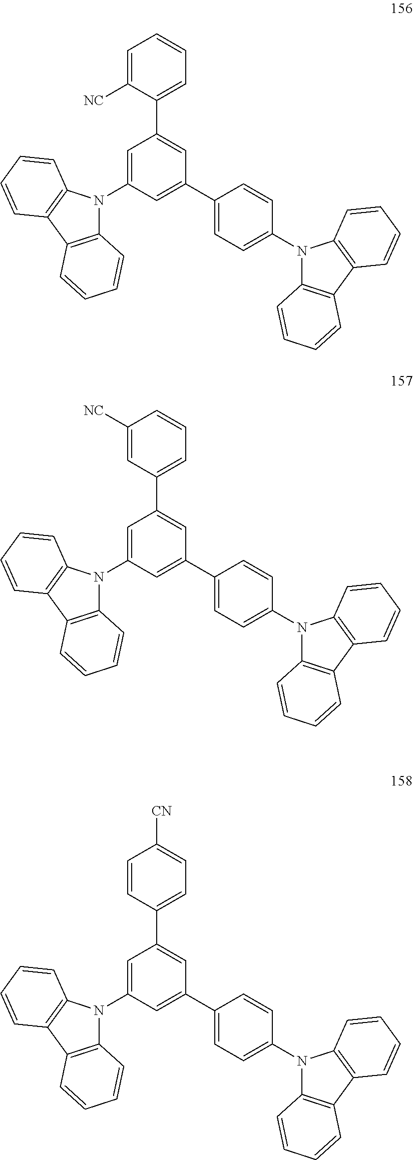 [SMILES: N#Cc1ccccc1-c1cc(-c2ccc(-n3c4ccccc4c4ccccc43)cc2)cc(-n2c3ccccc3c3ccccc32)c1 ; N#Cc1cccc(-c2cc(-c3ccc(-n4c5ccccc5c5ccccc54)cc3)cc(-n3c4ccccc4c4ccccc43)c2)c1 ; N#Cc1ccc(-c2cc(-c3ccc(-n4c5ccccc5c5ccccc54)cc3)cc(-n3c4ccccc4c4ccccc43)c2)cc1]

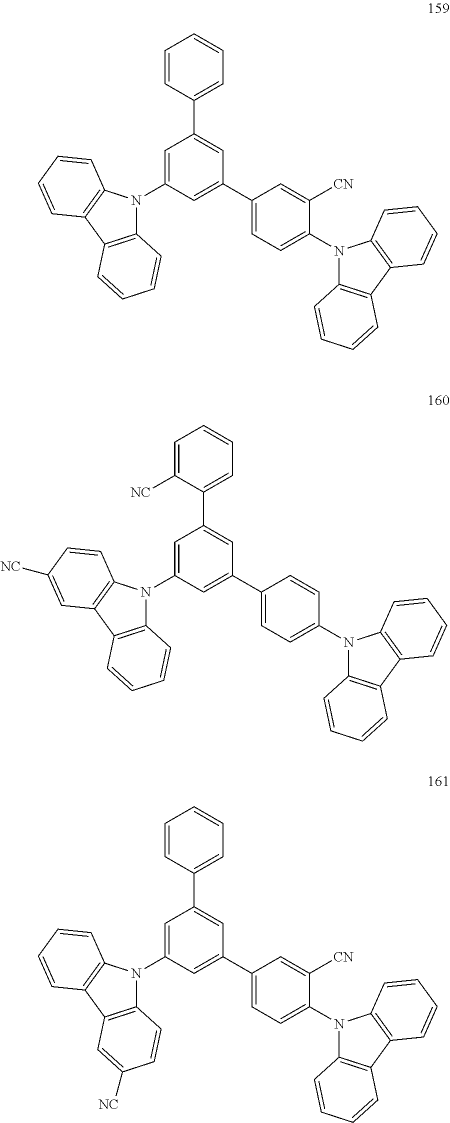 [SMILES: N#Cc1cc(-c2cc(-c3ccccc3)cc(-n3c4ccccc4c4ccccc43)c2)ccc1-n1c2ccccc2c2ccccc21 ; N#Cc1ccc2c(c1)c1ccccc1n2-c1cc(-c2ccc(-n3c4ccccc4c4ccccc43)cc2)cc(-c2ccccc2C#N)c1 ; N#Cc1ccc2c(c1)c1ccccc1n2-c1cc(-c2ccccc2)cc(-c2ccc(-n3c4ccccc4c4ccccc43)c(C#N)c2)c1]

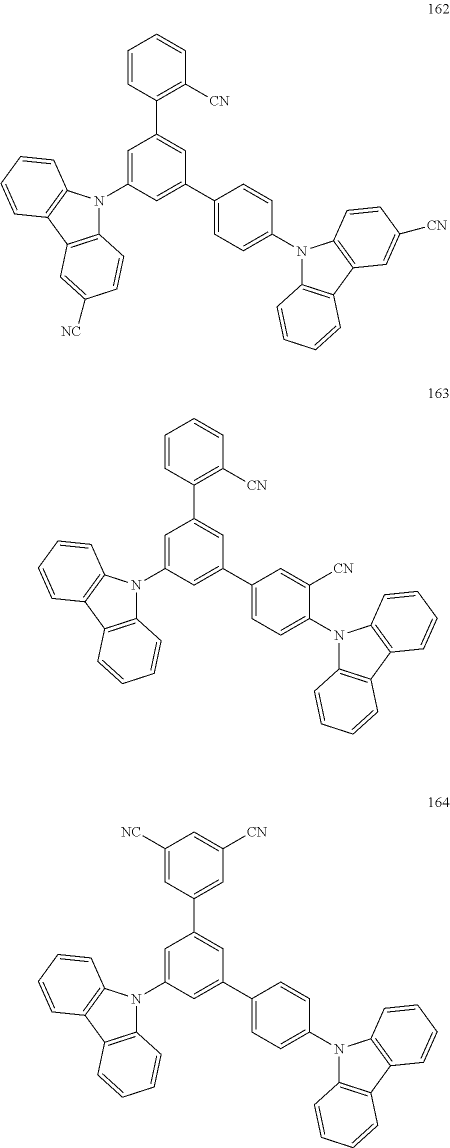 [SMILES: N#Cc1ccc2c(c1)c1ccccc1n2-c1ccc(-c2cc(-c3ccccc3C#N)cc(-n3c4ccccc4c4cc(C#N)ccc43)c2)cc1 ; N#Cc1ccccc1-c1cc(-c2ccc(-n3c4ccccc4c4ccccc43)c(C#N)c2)cc(-n2c3ccccc3c3ccccc32)c1 ; N#Cc1cc(C#N)cc(-c2cc(-c3ccc(-n4c5ccccc5c5ccccc54)cc3)cc(-n3c4ccccc4c4ccccc43)c2)c1]

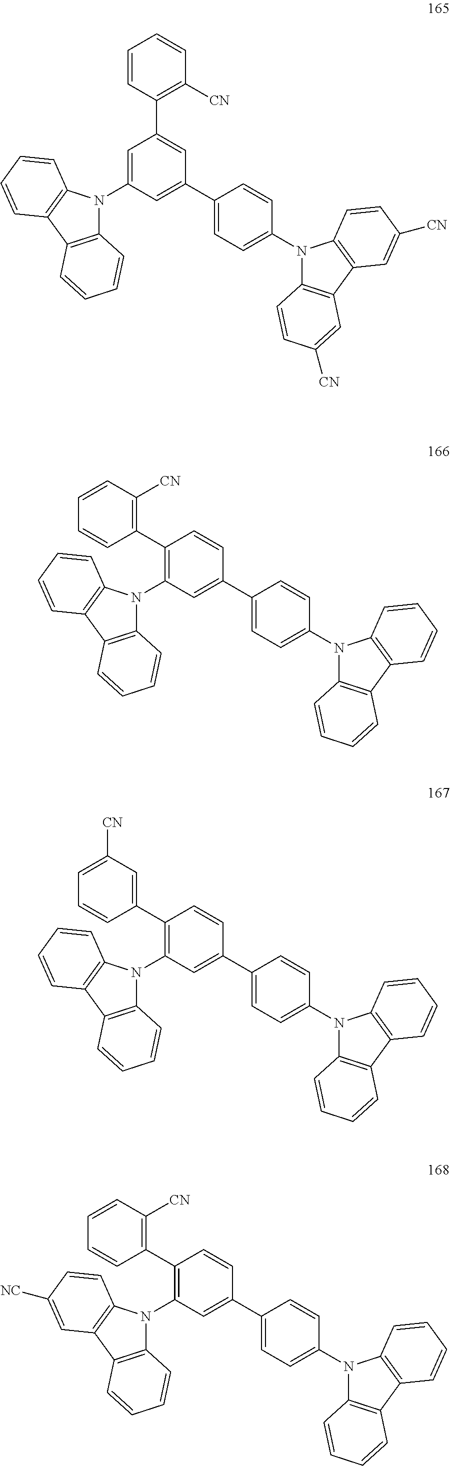 [SMILES: N#Cc1ccc2c(c1)c1cc(C#N)ccc1n2-c1ccc(-c2cc(-c3ccccc3C#N)cc(-n3c4ccccc4c4ccccc43)c2)cc1 ; N#Cc1ccccc1-c1ccc(-c2ccc(-n3c4ccccc4c4ccccc43)cc2)cc1-n1c2ccccc2c2ccccc21 ; N#Cc1cccc(-c2ccc(-c3ccc(-n4c5ccccc5c5ccccc54)cc3)cc2-n2c3ccccc3c3ccccc32)c1 ; N#Cc1ccc2c(c1)c1ccccc1n2-c1cc(-c2ccc(-n3c4ccccc4c4ccccc43)cc2)ccc1-c1ccccc1C#N]

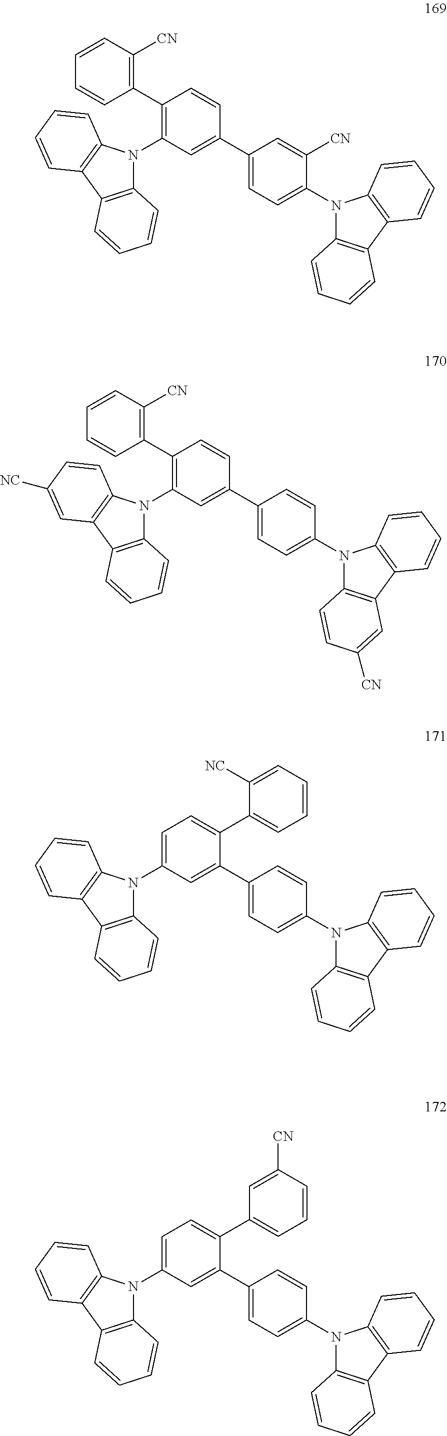 [SMILES: N#Cc1ccccc1-c1ccc(-c2ccc(-n3c4ccccc4c4ccccc43)c(C#N)c2)cc1-n1c2ccccc2c2ccccc21 ; N#Cc1ccc2c(c1)c1ccccc1n2-c1ccc(-c2ccc(-c3ccccc3C#N)c(-n3c4ccccc4c4cc(C#N)ccc43)c2)cc1 ; N#Cc1ccccc1-c1ccc(-n2c3ccccc3c3ccccc32)cc1-c1ccc(-n2c3ccccc3c3ccccc32)cc1 ; N#Cc1cccc(-c2ccc(-n3c4ccccc4c4ccccc43)cc2-c2ccc(-n3c4ccccc4c4ccccc43)cc2)c1]

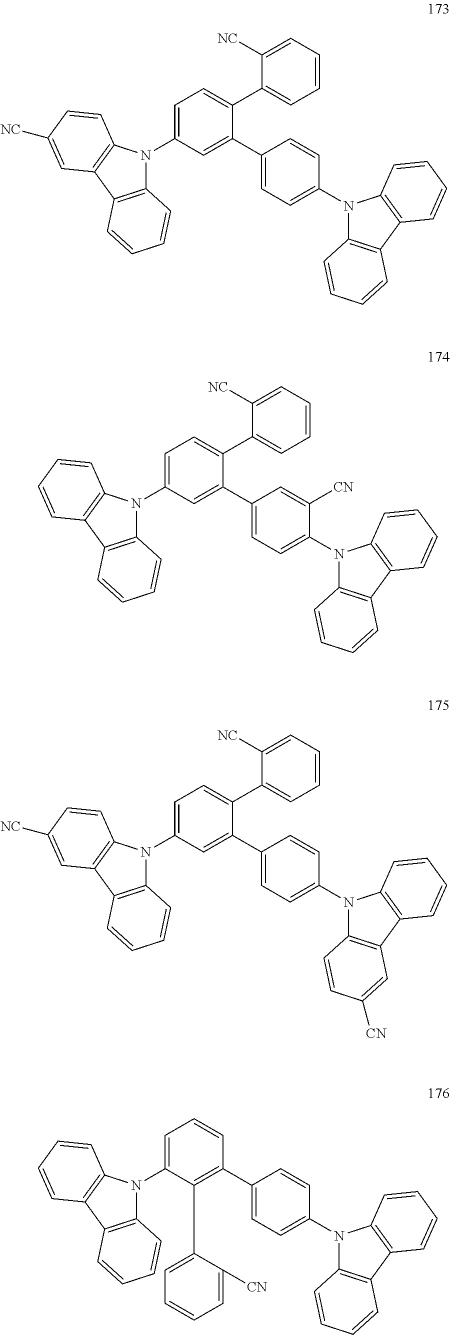 [SMILES: N#Cc1ccc2c(c1)c1ccccc1n2-c1ccc(-c2ccccc2C#N)c(-c2ccc(-n3c4ccccc4c4ccccc43)cc2)c1 ; N#Cc1ccccc1-c1ccc(-n2c3ccccc3c3ccccc32)cc1-c1ccc(-n2c3ccccc3c3ccccc32)c(C#N)c1 ; N#Cc1ccc2c(c1)c1ccccc1n2-c1ccc(-c2cc(-n3c4ccccc4c4cc(C#N)ccc43)ccc2-c2ccccc2C#N)cc1 ; N#Cc1ccccc1-c1c(-c2ccc(-n3c4ccccc4c4ccccc43)cc2)cccc1-n1c2ccccc2c2ccccc21]

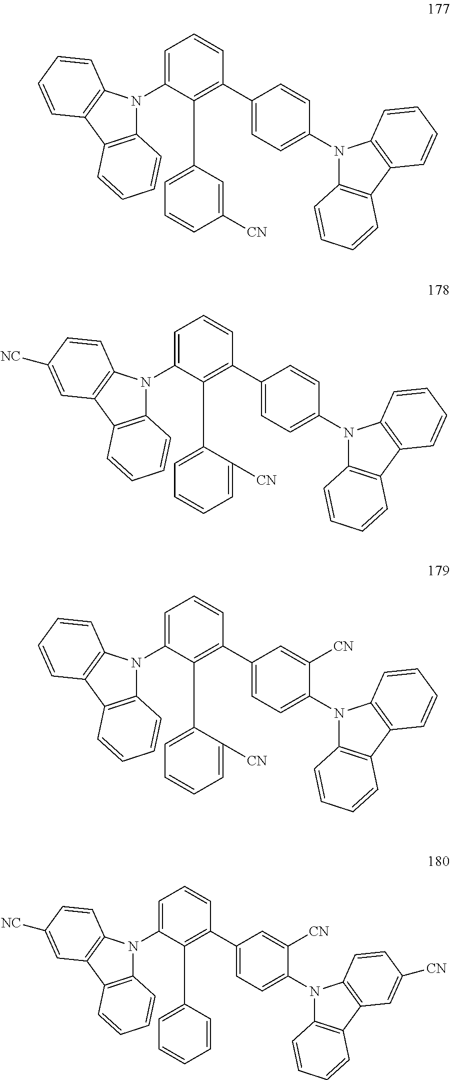 [SMILES: N#Cc1cccc(-c2c(-c3ccc(-n4c5ccccc5c5ccccc54)cc3)cccc2-n2c3ccccc3c3ccccc32)c1 ; N#Cc1ccc2c(c1)c1ccccc1n2-c1cccc(-c2ccc(-n3c4ccccc4c4ccccc43)cc2)c1-c1ccccc1C#N ; N#Cc1ccccc1-c1c(-c2ccc(-n3c4ccccc4c4ccccc43)c(C#N)c2)cccc1-n1c2ccccc2c2ccccc21 ; N#Cc1ccc2c(c1)c1ccccc1n2-c1ccc(-c2cccc(-n3c4ccccc4c4cc(C#N)ccc43)c2-c2ccccc2)cc1C#N]

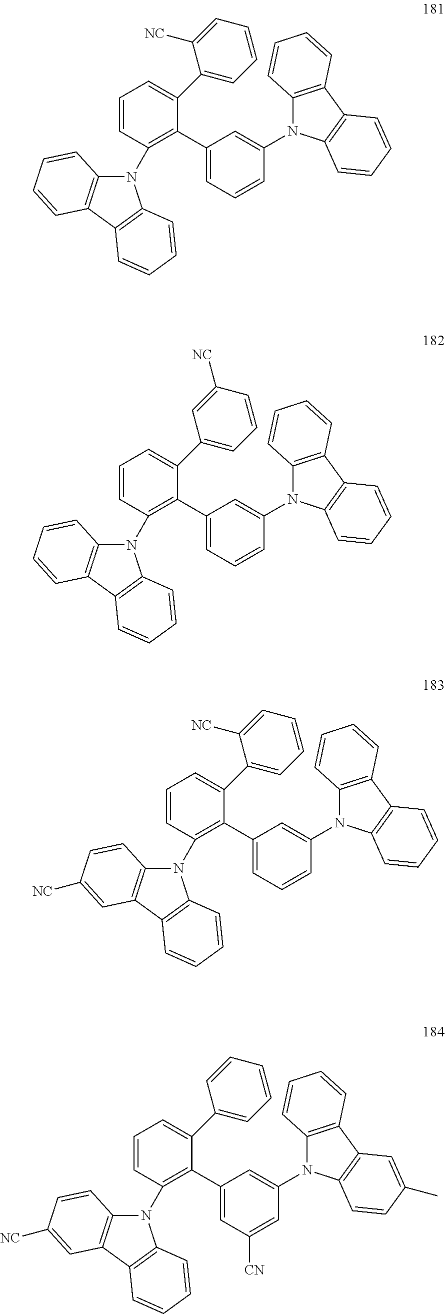 [SMILES: N#Cc1ccccc1-c1cccc(-n2c3ccccc3c3ccccc32)c1-c1cccc(-n2c3ccccc3c3ccccc32)c1 ; N#Cc1cccc(-c2cccc(-n3c4ccccc4c4ccccc43)c2-c2cccc(-n3c4ccccc4c4ccccc43)c2)c1 ; N#Cc1ccc2c(c1)c1ccccc1n2-c1cccc(-c2ccccc2C#N)c1-c1cccc(-n2c3ccccc3c3ccccc32)c1 ; Cc1ccc2c(c1)c1ccccc1n2-c1cc(C#N)cc(-c2c(-c3ccccc3)cccc2-n2c3ccccc3c3cc(C#N)ccc32)c1]

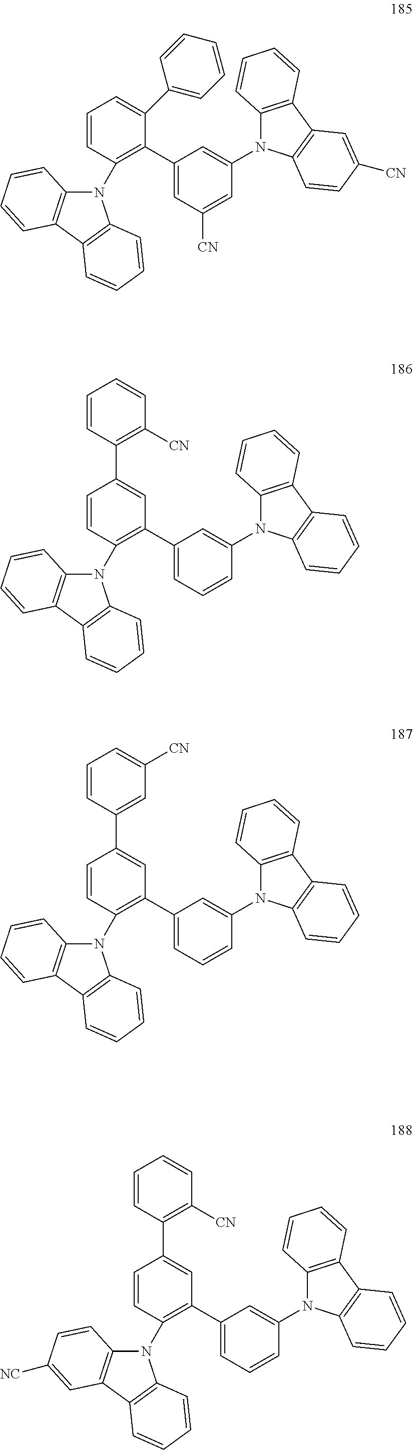 [SMILES: N#Cc1cc(-c2c(-c3ccccc3)cccc2-n2c3ccccc3c3ccccc32)cc(-n2c3ccccc3c3cc(C#N)ccc32)c1 ; N#Cc1ccccc1-c1ccc(-n2c3ccccc3c3ccccc32)c(-c2cccc(-n3c4ccccc4c4ccccc43)c2)c1 ; N#Cc1cccc(-c2ccc(-n3c4ccccc4c4ccccc43)c(-c3cccc(-n4c5ccccc5c5ccccc54)c3)c2)c1 ; N#Cc1ccc2c(c1)c1ccccc1n2-c1ccc(-c2ccccc2C#N)cc1-c1cccc(-n2c3ccccc3c3ccccc32)c1]

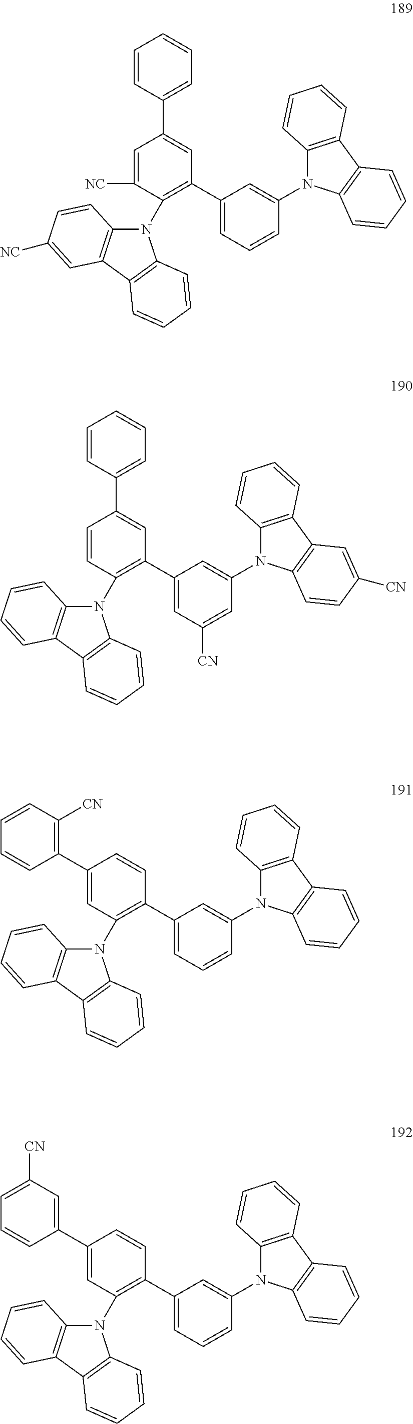 [SMILES: N#Cc1ccc2c(c1)c1ccccc1n2-c1c(C#N)cc(-c2ccccc2)cc1-c1cccc(-n2c3ccccc3c3ccccc32)c1 ; N#Cc1cc(-c2cc(-c3ccccc3)ccc2-n2c3ccccc3c3ccccc32)cc(-n2c3ccccc3c3cc(C#N)ccc32)c1 ; N#Cc1ccccc1-c1ccc(-c2cccc(-n3c4ccccc4c4ccccc43)c2)c(-n2c3ccccc3c3ccccc32)c1 ; N#Cc1cccc(-c2ccc(-c3cccc(-n4c5ccccc5c5ccccc54)c3)c(-n3c4ccccc4c4ccccc43)c2)c1]

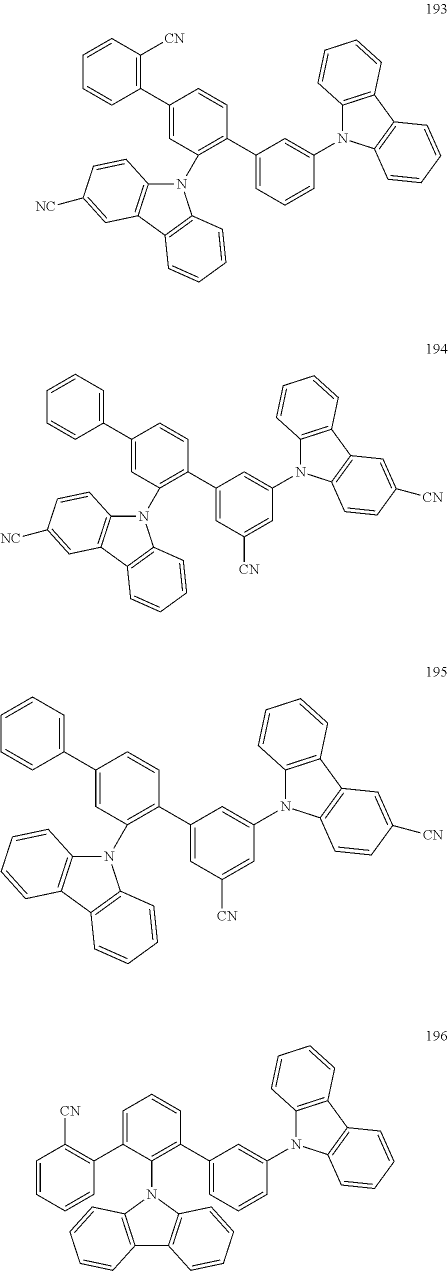 [SMILES: N#Cc1ccc2c(c1)c1ccccc1n2-c1cc(-c2ccccc2C#N)ccc1-c1cccc(-n2c3ccccc3c3ccccc32)c1 ; N#Cc1cc(-c2ccc(-c3ccccc3)cc2-n2c3ccccc3c3cc(C#N)ccc32)cc(-n2c3ccccc3c3cc(C#N)ccc32)c1 ; N#Cc1cc(-c2ccc(-c3ccccc3)cc2-n2c3ccccc3c3ccccc32)cc(-n2c3ccccc3c3cc(C#N)ccc32)c1 ; N#Cc1ccccc1-c1cccc(-c2cccc(-n3c4ccccc4c4ccccc43)c2)c1-n1c2ccccc2c2ccccc21]

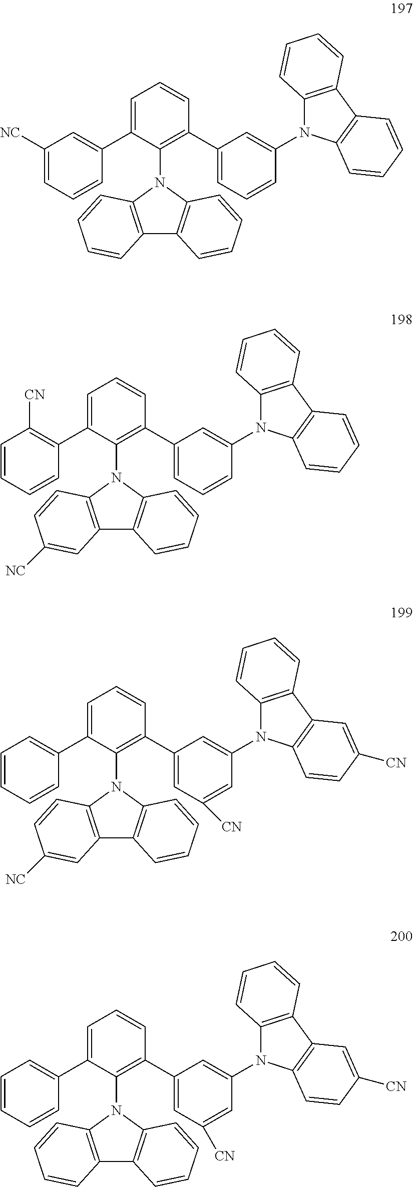 [SMILES: N#Cc1cccc(-c2cccc(-c3cccc(-n4c5ccccc5c5ccccc54)c3)c2-n2c3ccccc3c3ccccc32)c1 ; N#Cc1ccc2c(c1)c1ccccc1n2-c1c(-c2cccc(-n3c4ccccc4c4ccccc43)c2)cccc1-c1ccccc1C#N ; N#Cc1cc(-c2cccc(-c3ccccc3)c2-n2c3ccccc3c3cc(C#N)ccc32)cc(-n2c3ccccc3c3cc(C#N)ccc32)c1 ; N#Cc1cc(-c2cccc(-c3ccccc3)c2-n2c3ccccc3c3ccccc32)cc(-n2c3ccccc3c3cc(C#N)ccc32)c1]

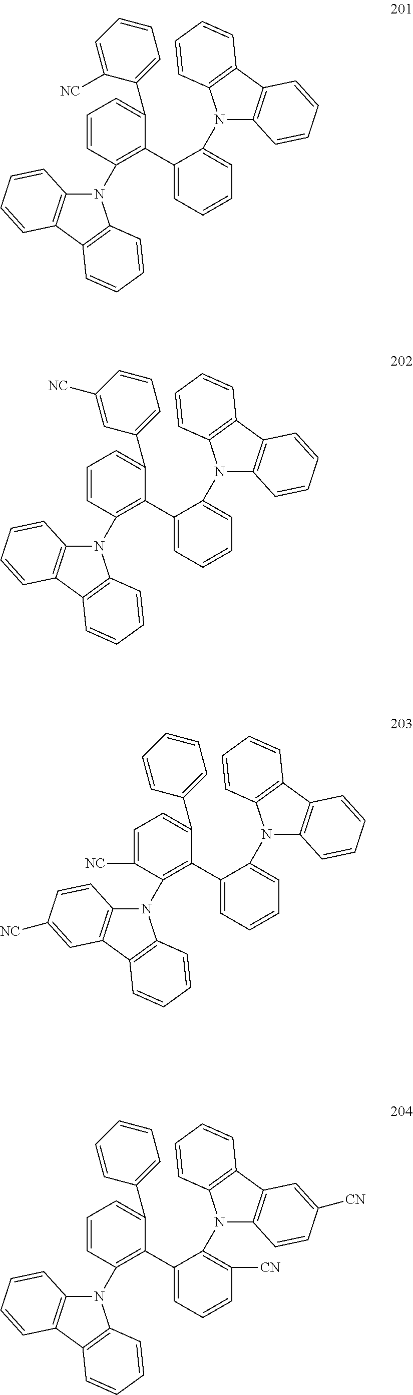 [SMILES: N#Cc1ccccc1-c1cccc(-n2c3ccccc3c3ccccc32)c1-c1ccccc1-n1c2ccccc2c2ccccc21 ; N#Cc1cccc(-c2cccc(-n3c4ccccc4c4ccccc43)c2-c2ccccc2-n2c3ccccc3c3ccccc32)c1 ; N#Cc1ccc2c(c1)c1ccccc1n2-c1c(C#N)ccc(-c2ccccc2)c1-c1ccccc1-n1c2ccccc2c2ccccc21 ; N#Cc1ccc2c(c1)c1ccccc1n2-c1c(C#N)cccc1-c1c(-c2ccccc2)cccc1-n1c2ccccc2c2ccccc21]

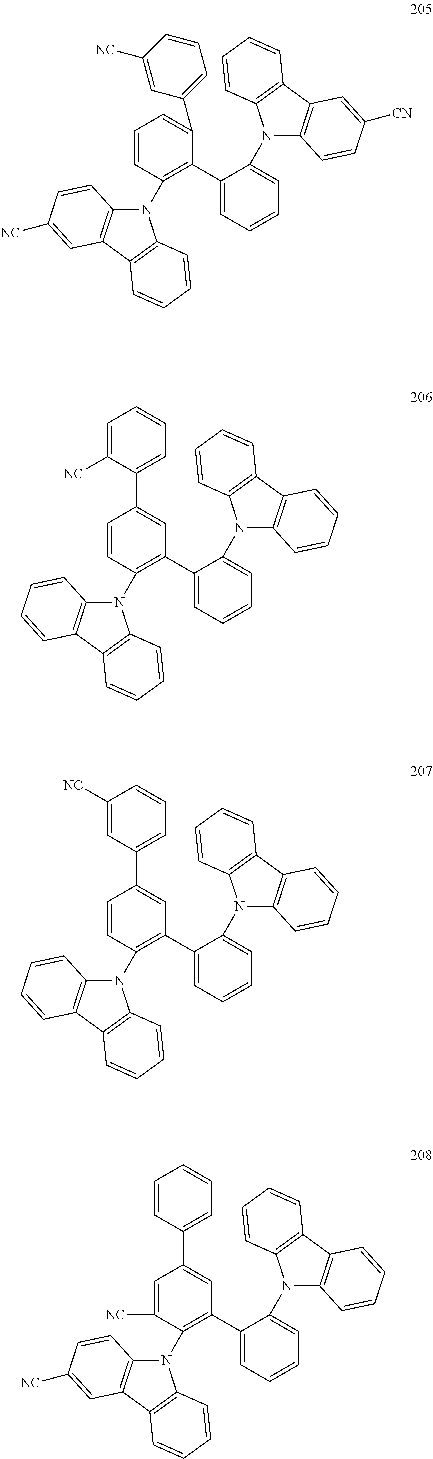 [SMILES: N#Cc1cccc(-c2cccc(-n3c4ccccc4c4cc(C#N)ccc43)c2-c2ccccc2-n2c3ccccc3c3cc(C#N)ccc32)c1 ; N#Cc1ccccc1-c1ccc(-n2c3ccccc3c3ccccc32)c(-c2ccccc2-n2c3ccccc3c3ccccc32)c1 ; N#Cc1cccc(-c2ccc(-n3c4ccccc4c4ccccc43)c(-c3ccccc3-n3c4ccccc4c4ccccc43)c2)c1 ; N#Cc1ccc2c(c1)c1ccccc1n2-c1c(C#N)cc(-c2ccccc2)cc1-c1ccccc1-n1c2ccccc2c2ccccc21]

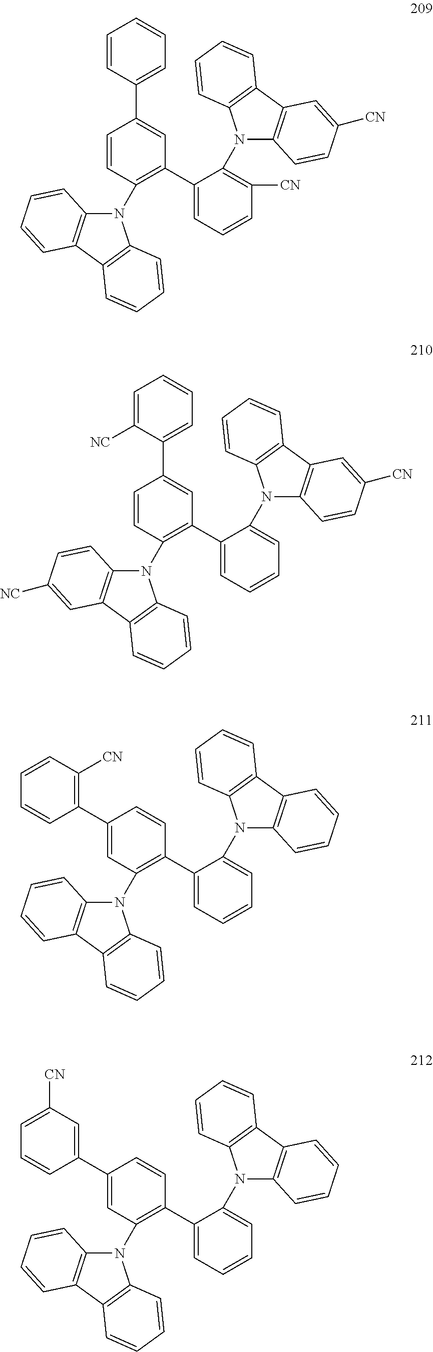 [SMILES: N#Cc1ccc2c(c1)c1ccccc1n2-c1c(C#N)cccc1-c1cc(-c2ccccc2)ccc1-n1c2ccccc2c2ccccc21 ; N#Cc1ccc2c(c1)c1ccccc1n2-c1ccccc1-c1cc(-c2ccccc2C#N)ccc1-n1c2ccccc2c2cc(C#N)ccc21 ; N#Cc1ccccc1-c1ccc(-c2ccccc2-n2c3ccccc3c3ccccc32)c(-n2c3ccccc3c3ccccc32)c1 ; N#Cc1cccc(-c2ccc(-c3ccccc3-n3c4ccccc4c4ccccc43)c(-n3c4ccccc4c4ccccc43)c2)c1]

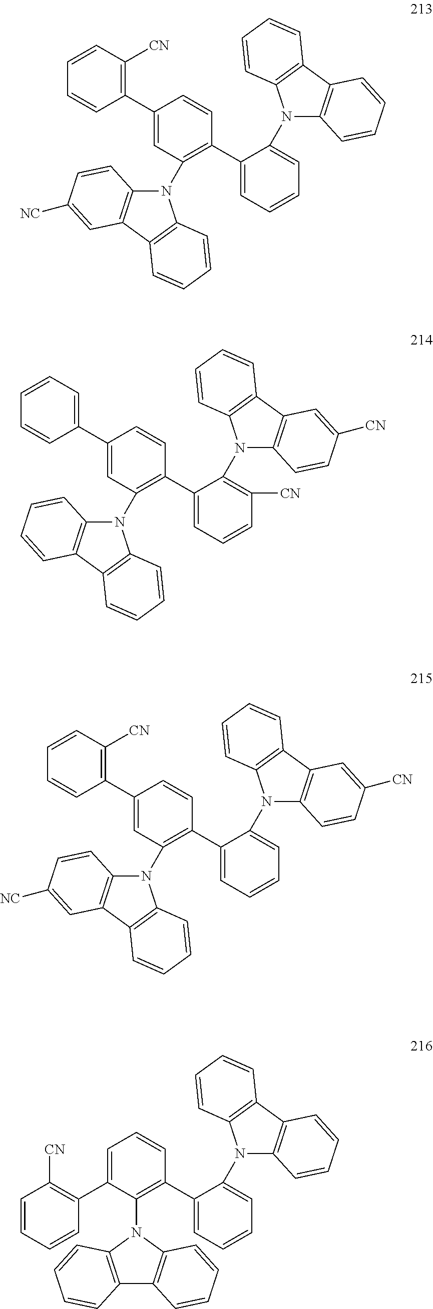 [SMILES: N#Cc1ccc2c(c1)c1ccccc1n2-c1cc(-c2ccccc2C#N)ccc1-c1ccccc1-n1c2ccccc2c2ccccc21 ; N#Cc1ccc2c(c1)c1ccccc1n2-c1c(C#N)cccc1-c1ccc(-c2ccccc2)cc1-n1c2ccccc2c2ccccc21 ; N#Cc1ccc2c(c1)c1ccccc1n2-c1ccccc1-c1ccc(-c2ccccc2C#N)cc1-n1c2ccccc2c2cc(C#N)ccc21 ; N#Cc1ccccc1-c1cccc(-c2ccccc2-n2c3ccccc3c3ccccc32)c1-n1c2ccccc2c2ccccc21]

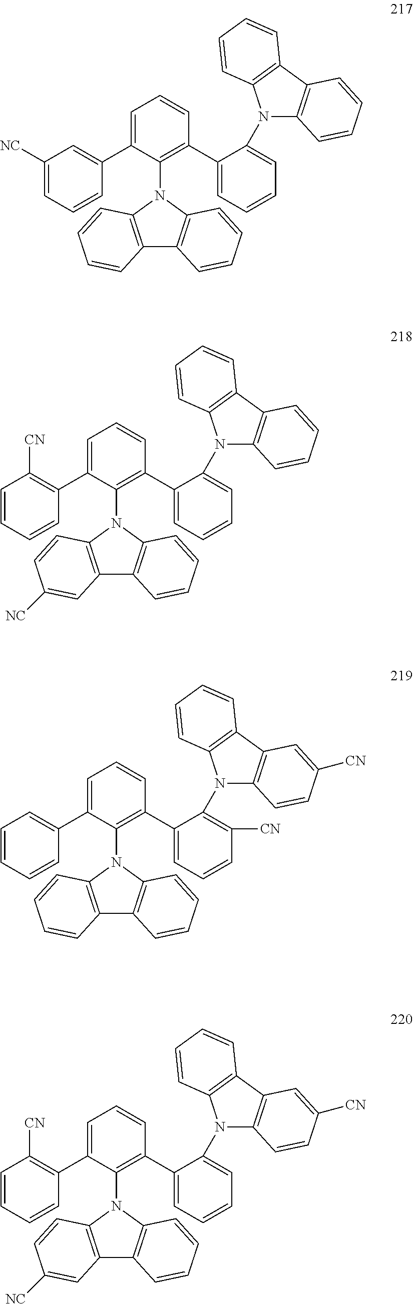 [SMILES: N#Cc1cccc(-c2cccc(-c3ccccc3-n3c4ccccc4c4ccccc43)c2-n2c3ccccc3c3ccccc32)c1 ; N#Cc1ccc2c(c1)c1ccccc1n2-c1c(-c2ccccc2C#N)cccc1-c1ccccc1-n1c2ccccc2c2ccccc21 ; N#Cc1ccc2c(c1)c1ccccc1n2-c1c(C#N)cccc1-c1cccc(-c2ccccc2)c1-n1c2ccccc2c2ccccc21 ; N#Cc1ccc2c(c1)c1ccccc1n2-c1ccccc1-c1cccc(-c2ccccc2C#N)c1-n1c2ccccc2c2cc(C#N)ccc21]

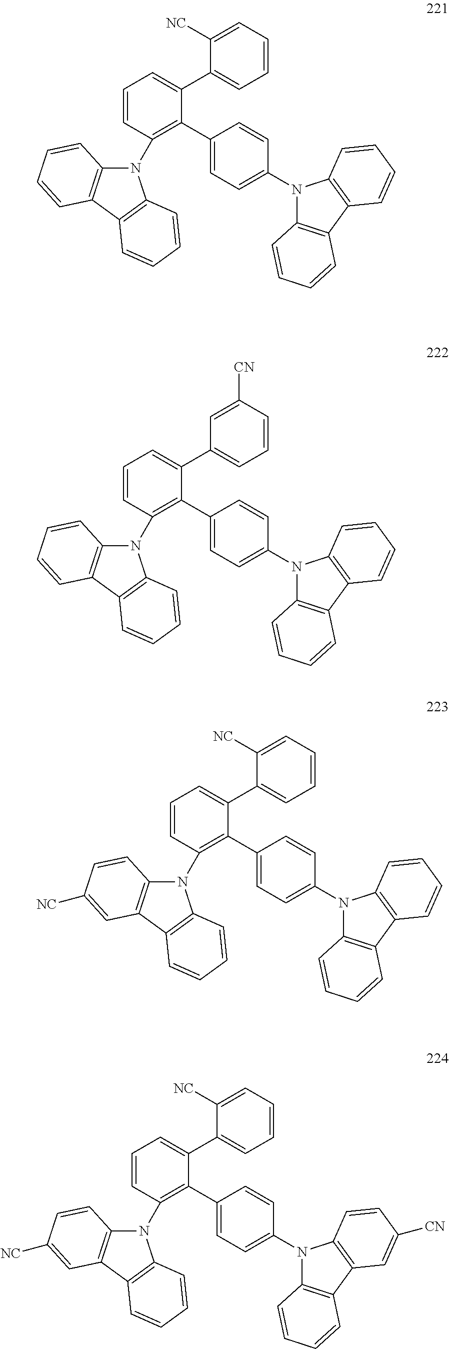 [SMILES: N#Cc1ccccc1-c1cccc(-n2c3ccccc3c3ccccc32)c1-c1ccc(-n2c3ccccc3c3ccccc32)cc1 ; N#Cc1cccc(-c2cccc(-n3c4ccccc4c4ccccc43)c2-c2ccc(-n3c4ccccc4c4ccccc43)cc2)c1 ; N#Cc1ccc2c(c1)c1ccccc1n2-c1cccc(-c2ccccc2C#N)c1-c1ccc(-n2c3ccccc3c3ccccc32)cc1 ; N#Cc1ccc2c(c1)c1ccccc1n2-c1ccc(-c2c(-c3ccccc3C#N)cccc2-n2c3ccccc3c3cc(C#N)ccc32)cc1]

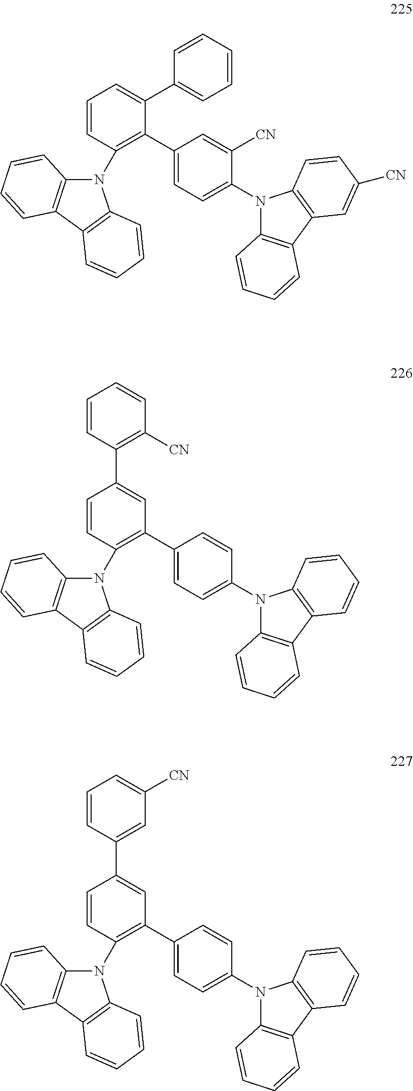 [SMILES: N#Cc1ccc2c(c1)c1ccccc1n2-c1ccc(-c2c(-c3ccccc3)cccc2-n2c3ccccc3c3ccccc32)cc1C#N ; N#Cc1ccccc1-c1ccc(-n2c3ccccc3c3ccccc32)c(-c2ccc(-n3c4ccccc4c4ccccc43)cc2)c1 ; N#Cc1cccc(-c2ccc(-n3c4ccccc4c4ccccc43)c(-c3ccc(-n4c5ccccc5c5ccccc54)cc3)c2)c1]

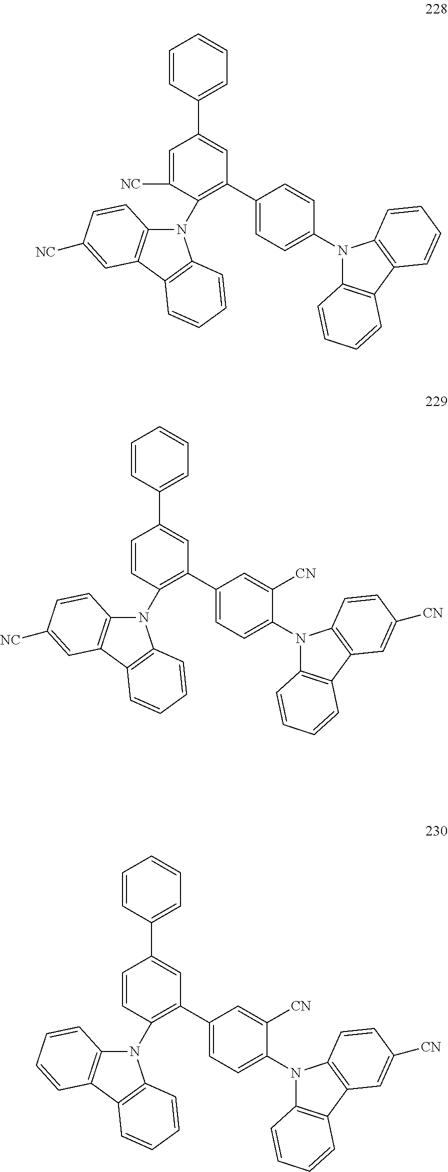 [SMILES: N#Cc1ccc2c(c1)c1ccccc1n2-c1c(C#N)cc(-c2ccccc2)cc1-c1ccc(-n2c3ccccc3c3ccccc32)cc1 ; N#Cc1ccc2c(c1)c1ccccc1n2-c1ccc(-c2cc(-c3ccccc3)ccc2-n2c3ccccc3c3cc(C#N)ccc32)cc1C#N ; N#Cc1ccc2c(c1)c1ccccc1n2-c1ccc(-c2cc(-c3ccccc3)ccc2-n2c3ccccc3c3ccccc32)cc1C#N]

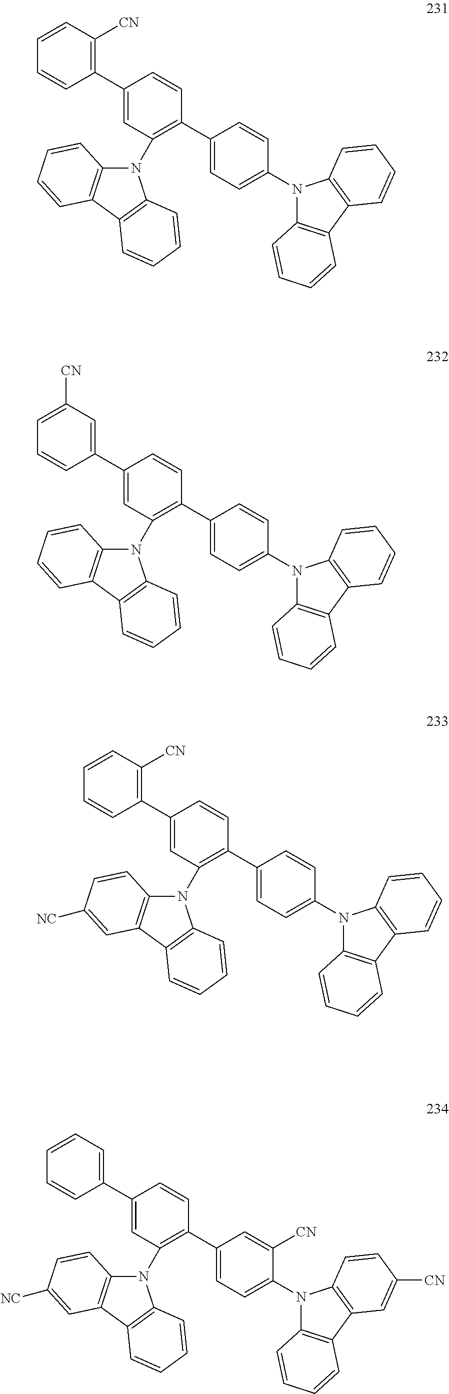 [SMILES: N#Cc1ccccc1-c1ccc(-c2ccc(-n3c4ccccc4c4ccccc43)cc2)c(-n2c3ccccc3c3ccccc32)c1 ; N#Cc1cccc(-c2ccc(-c3ccc(-n4c5ccccc5c5ccccc54)cc3)c(-n3c4ccccc4c4ccccc43)c2)c1 ; N#Cc1ccc2c(c1)c1ccccc1n2-c1cc(-c2ccccc2C#N)ccc1-c1ccc(-n2c3ccccc3c3ccccc32)cc1 ; N#Cc1ccc2c(c1)c1ccccc1n2-c1ccc(-c2ccc(-c3ccccc3)cc2-n2c3ccccc3c3cc(C#N)ccc32)cc1C#N]

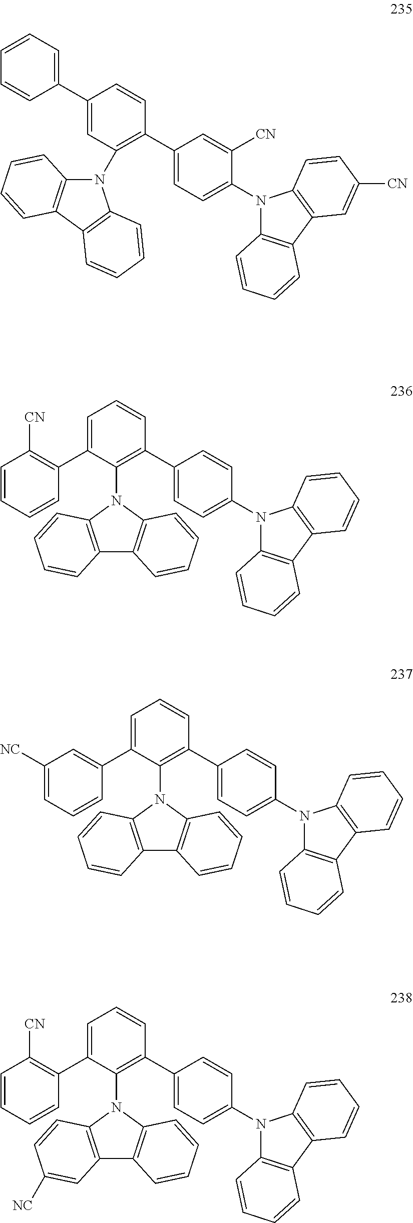 [SMILES: N#Cc1ccc2c(c1)c1ccccc1n2-c1ccc(-c2ccc(-c3ccccc3)cc2-n2c3ccccc3c3ccccc32)cc1C#N ; N#Cc1ccccc1-c1cccc(-c2ccc(-n3c4ccccc4c4ccccc43)cc2)c1-n1c2ccccc2c2ccccc21 ; N#Cc1cccc(-c2cccc(-c3ccc(-n4c5ccccc5c5ccccc54)cc3)c2-n2c3ccccc3c3ccccc32)c1 ; N#Cc1ccc2c(c1)c1ccccc1n2-c1c(-c2ccc(-n3c4ccccc4c4ccccc43)cc2)cccc1-c1ccccc1C#N]

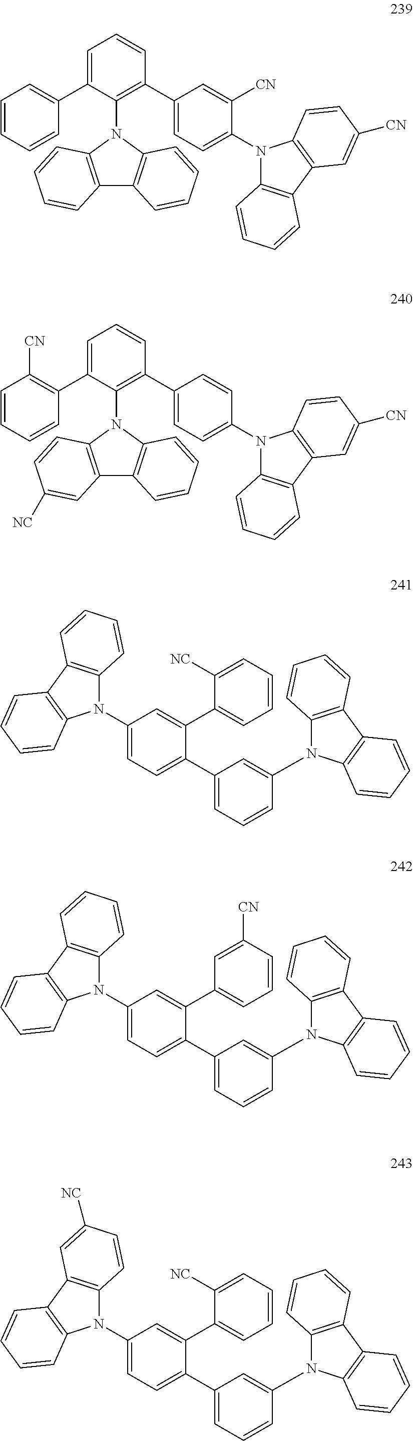 [SMILES: N#Cc1ccc2c(c1)c1ccccc1n2-c1ccc(-c2cccc(-c3ccccc3)c2-n2c3ccccc3c3ccccc32)cc1C#N ; N#Cc1ccc2c(c1)c1ccccc1n2-c1ccc(-c2cccc(-c3ccccc3C#N)c2-n2c3ccccc3c3cc(C#N)ccc32)cc1 ; N#Cc1ccccc1-c1cc(-n2c3ccccc3c3ccccc32)ccc1-c1cccc(-n2c3ccccc3c3ccccc32)c1 ; N#Cc1cccc(-c2cc(-n3c4ccccc4c4ccccc43)ccc2-c2cccc(-n3c4ccccc4c4ccccc43)c2)c1 ; N#Cc1ccc2c(c1)c1ccccc1n2-c1ccc(-c2cccc(-n3c4ccccc4c4ccccc43)c2)c(-c2ccccc2C#N)c1]

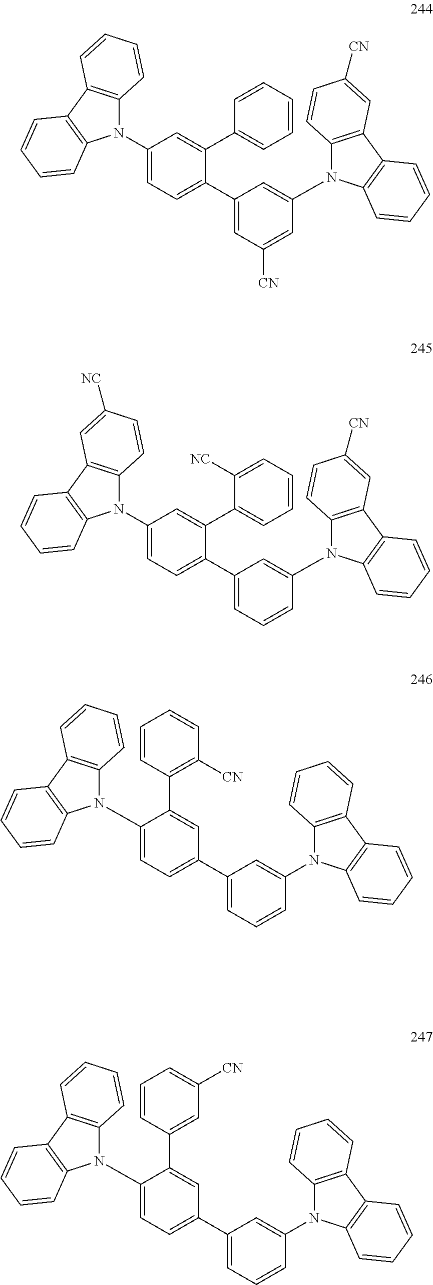 [SMILES: N#Cc1cc(-c2ccc(-n3c4ccccc4c4ccccc43)cc2-c2ccccc2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)cc2-c2ccccc2C#N)c1 ; N#Cc1ccccc1-c1cc(-c2cccc(-n3c4ccccc4c4ccccc43)c2)ccc1-n1c2ccccc2c2ccccc21 ; N#Cc1cccc(-c2cc(-c3cccc(-n4c5ccccc5c5ccccc54)c3)ccc2-n2c3ccccc3c3ccccc32)c1]

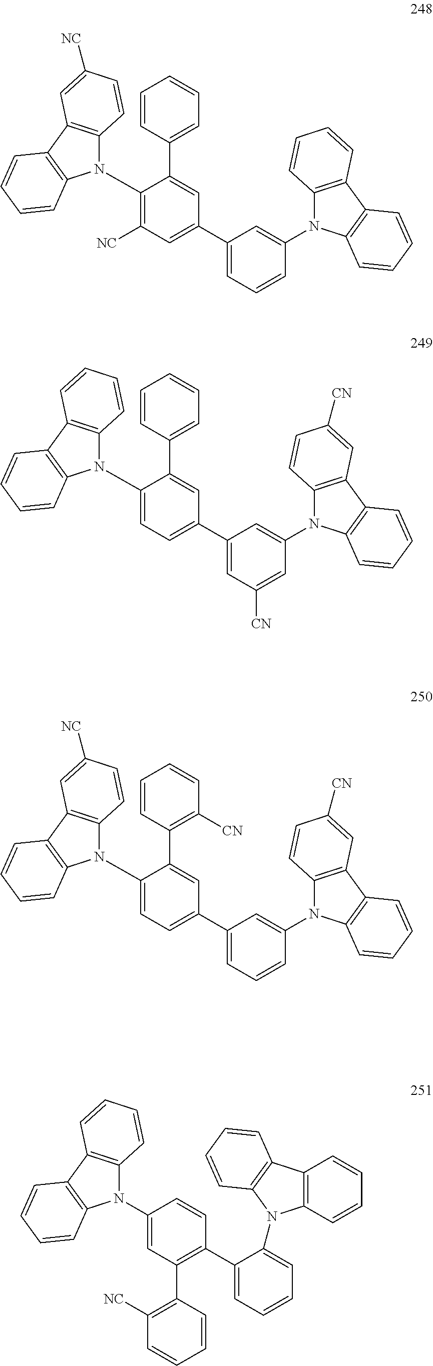 [SMILES: N#Cc1ccc2c(c1)c1ccccc1n2-c1c(C#N)cc(-c2cccc(-n3c4ccccc4c4ccccc43)c2)cc1-c1ccccc1 ; N#Cc1cc(-c2ccc(-n3c4ccccc4c4ccccc43)c(-c3ccccc3)c2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)c(-c3ccccc3C#N)c2)c1 ; N#Cc1ccccc1-c1cc(-n2c3ccccc3c3ccccc32)ccc1-c1ccccc1-n1c2ccccc2c2ccccc21]

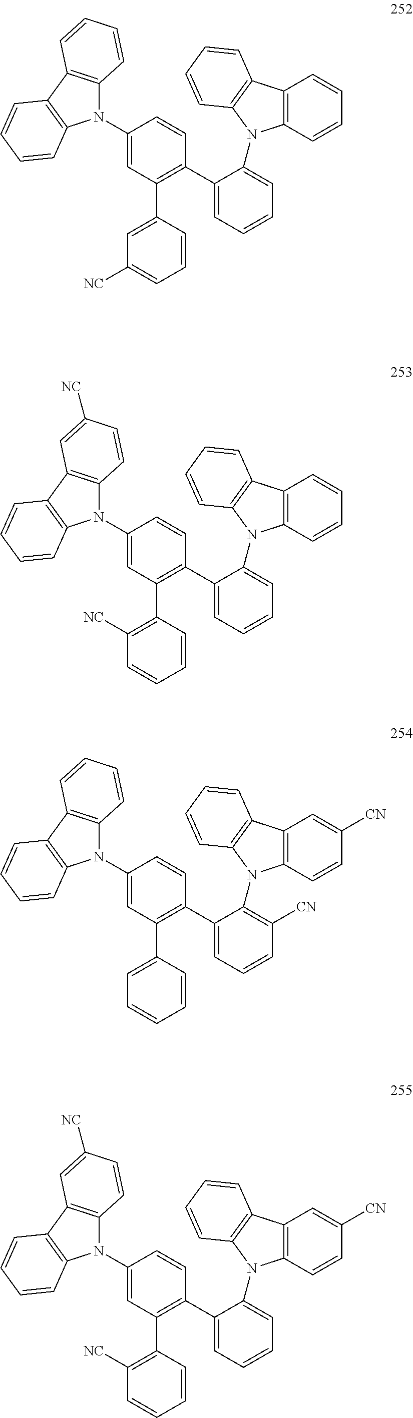 [SMILES: N#Cc1cccc(-c2cc(-n3c4ccccc4c4ccccc43)ccc2-c2ccccc2-n2c3ccccc3c3ccccc32)c1 ; N#Cc1ccc2c(c1)c1ccccc1n2-c1ccc(-c2ccccc2-n2c3ccccc3c3ccccc32)c(-c2ccccc2C#N)c1 ; N#Cc1ccc2c(c1)c1ccccc1n2-c1c(C#N)cccc1-c1ccc(-n2c3ccccc3c3ccccc32)cc1-c1ccccc1 ; N#Cc1ccc2c(c1)c1ccccc1n2-c1ccc(-c2ccccc2-n2c3ccccc3c3cc(C#N)ccc32)c(-c2ccccc2C#N)c1]

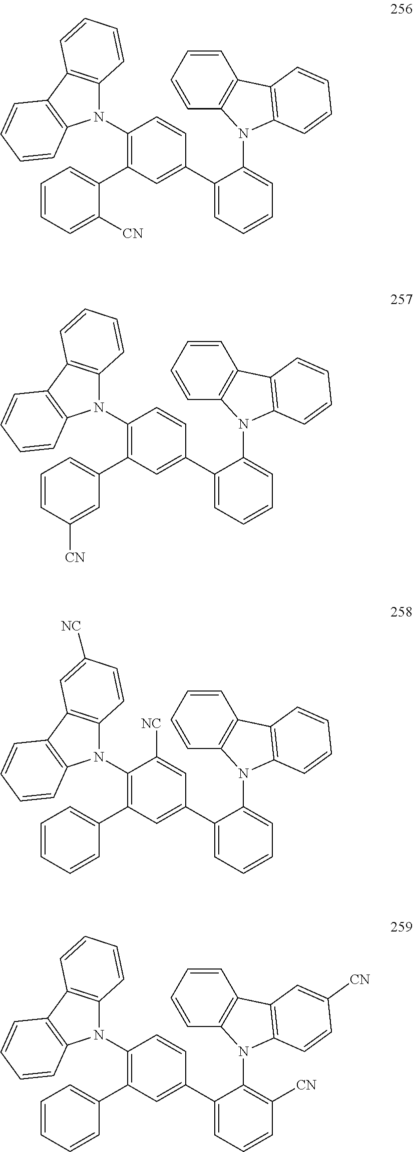 [SMILES: N#Cc1ccccc1-c1cc(-c2ccccc2-n2c3ccccc3c3ccccc32)ccc1-n1c2ccccc2c2ccccc21 ; N#Cc1cccc(-c2cc(-c3ccccc3-n3c4ccccc4c4ccccc43)ccc2-n2c3ccccc3c3ccccc32)c1 ; N#Cc1ccc2c(c1)c1ccccc1n2-c1c(C#N)cc(-c2ccccc2-n2c3ccccc3c3ccccc32)cc1-c1ccccc1 ; N#Cc1ccc2c(c1)c1ccccc1n2-c1c(C#N)cccc1-c1ccc(-n2c3ccccc3c3ccccc32)c(-c2ccccc2)c1]

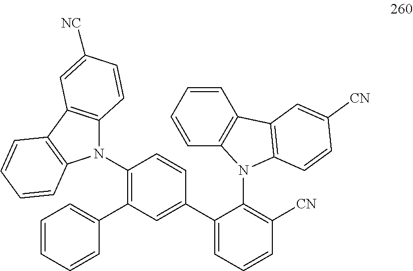 [SMILES: N#Cc1ccc2c(c1)c1ccccc1n2-c1ccc(-c2cccc(C#N)c2-n2c3ccccc3c3cc(C#N)ccc32)cc1-c1ccccc1]

In an embodiment, the electron-transporting host may include DPEPO and TSPO1:

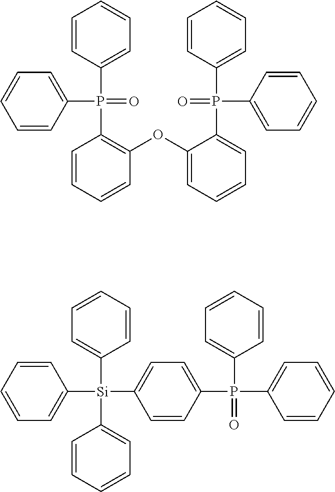 [SMILES: O=P(c1ccccc1)(c1ccccc1)c1ccccc1Oc1ccccc1P(=O)(c1ccccc1)c1ccccc1 ; O=P(c1ccccc1)(c1ccccc1)c1ccc([Si](c2ccccc2)(c2ccccc2)c2ccccc2)cc1]

In some embodiments, the hole transporting host may be a compound of Group HH1, but embodiments are not limited thereto:

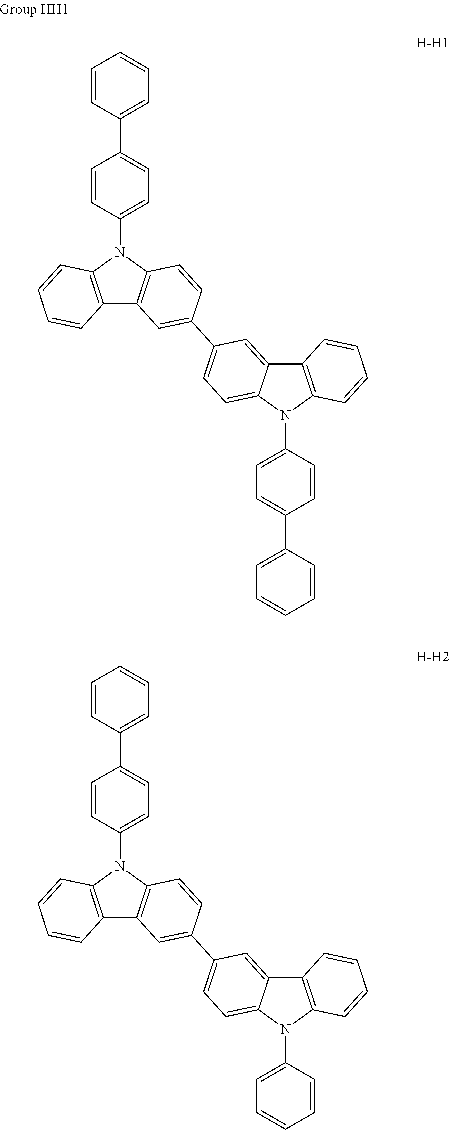 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccccc5)ccc43)cc2)cc1]

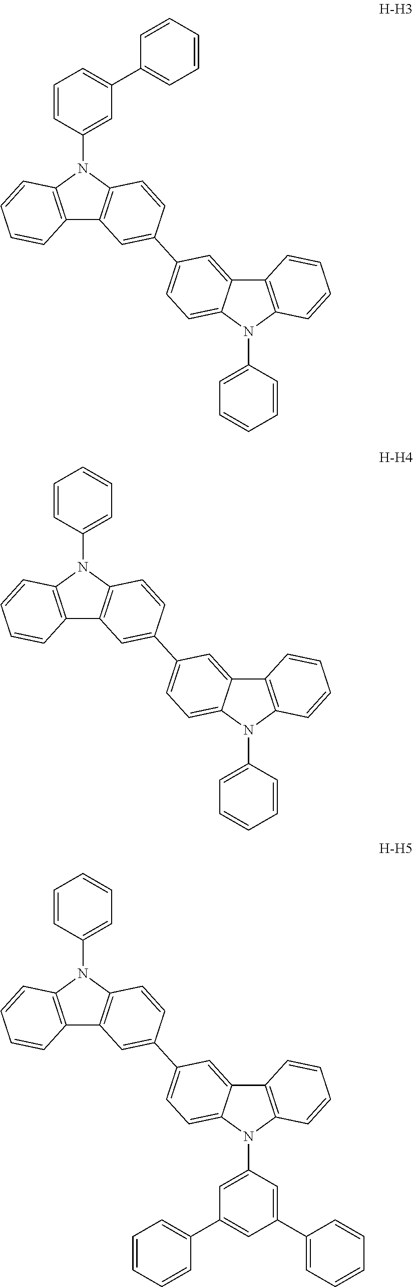 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccccc4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1]

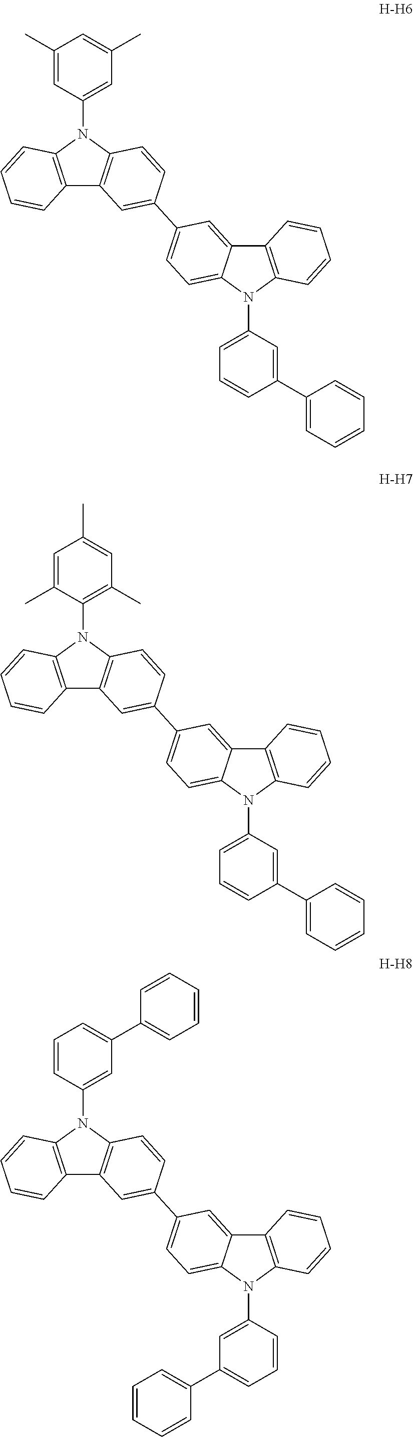 [SMILES: Cc1cc(C)cc(-n2c3ccccc3c3cc(-c4ccc5c(c4)c4ccccc4n5-c4cccc(-c5ccccc5)c4)ccc32)c1 ; Cc1cc(C)c(-n2c3ccccc3c3cc(-c4ccc5c(c4)c4ccccc4n5-c4cccc(-c5ccccc5)c4)ccc32)c(C)c1 ; c1ccc(-c2cccc(-n3c4ccccc4c4cc(-c5ccc6c(c5)c5ccccc5n6-c5cccc(-c6ccccc6)c5)ccc43)c2)cc1]

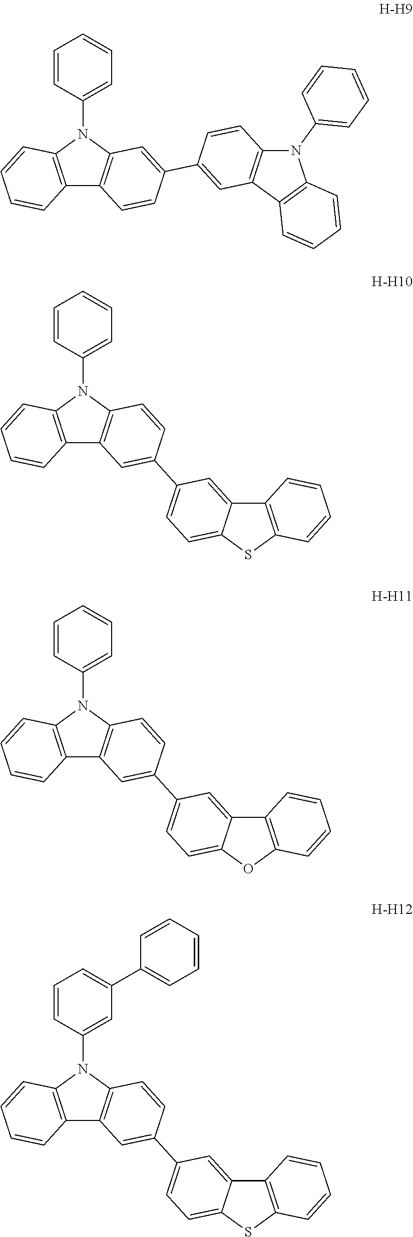 [SMILES: c1ccc(-n2c3ccccc3c3cc(-c4ccc5c6ccccc6n(-c6ccccc6)c5c4)ccc32)cc1 ; c1ccc(-n2c3ccccc3c3cc(-c4ccc5sc6ccccc6c5c4)ccc32)cc1 ; c1ccc(-n2c3ccccc3c3cc(-c4ccc5oc6ccccc6c5c4)ccc32)cc1 ; c1ccc(-c2cccc(-n3c4ccccc4c4cc(-c5ccc6sc7ccccc7c6c5)ccc43)c2)cc1]

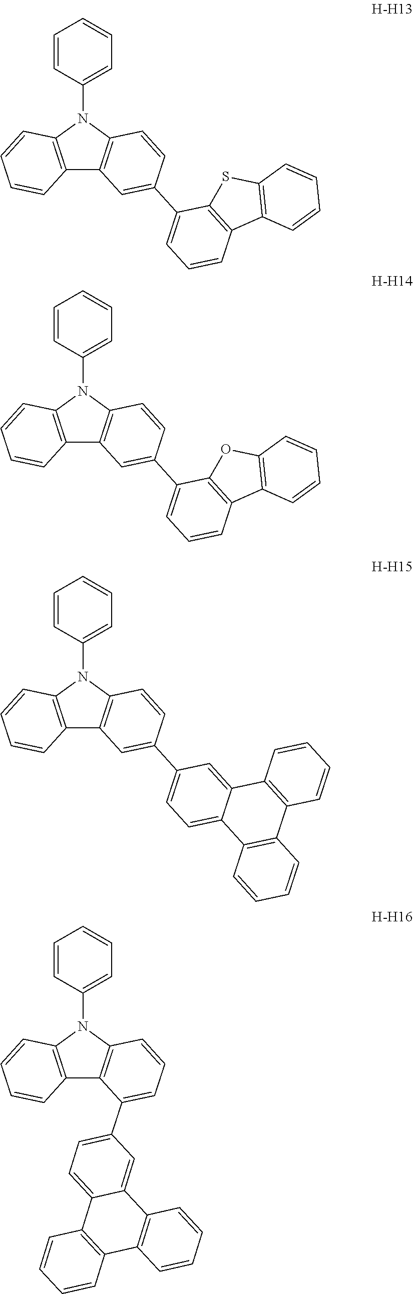 [SMILES: c1ccc(-n2c3ccccc3c3cc(-c4cccc5c4sc4ccccc45)ccc32)cc1 ; c1ccc(-n2c3ccccc3c3cc(-c4cccc5c4oc4ccccc45)ccc32)cc1 ; c1ccc(-n2c3ccccc3c3cc(-c4ccc5c6ccccc6c6ccccc6c5c4)ccc32)cc1 ; c1ccc(-n2c3ccccc3c3c(-c4ccc5c6ccccc6c6ccccc6c5c4)cccc32)cc1]

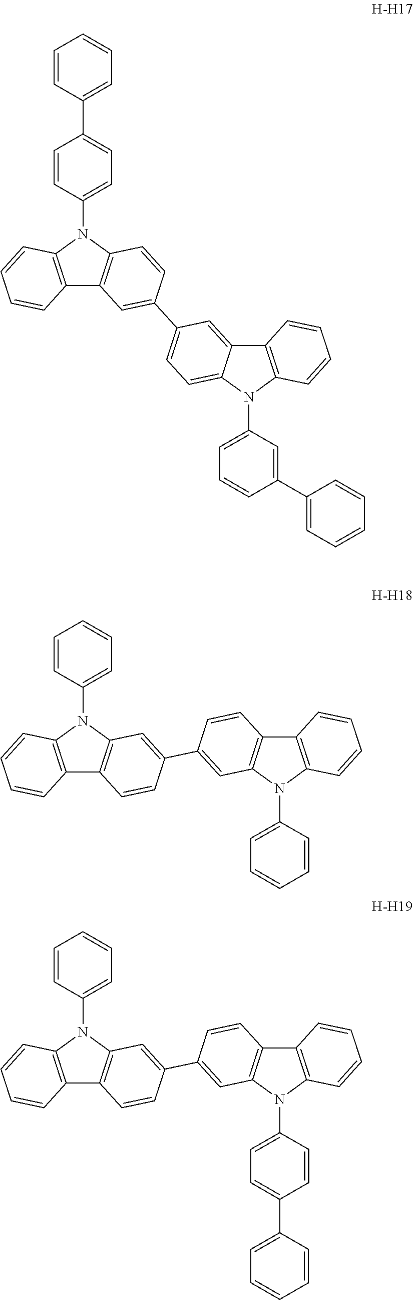 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3ccc(-c4ccc5c6ccccc6n(-c6ccccc6)c5c4)cc32)cc1 ; c1ccc(-c2ccc(-n3c4ccccc4c4ccc(-c5ccc6c7ccccc7n(-c7ccccc7)c6c5)cc43)cc2)cc1]

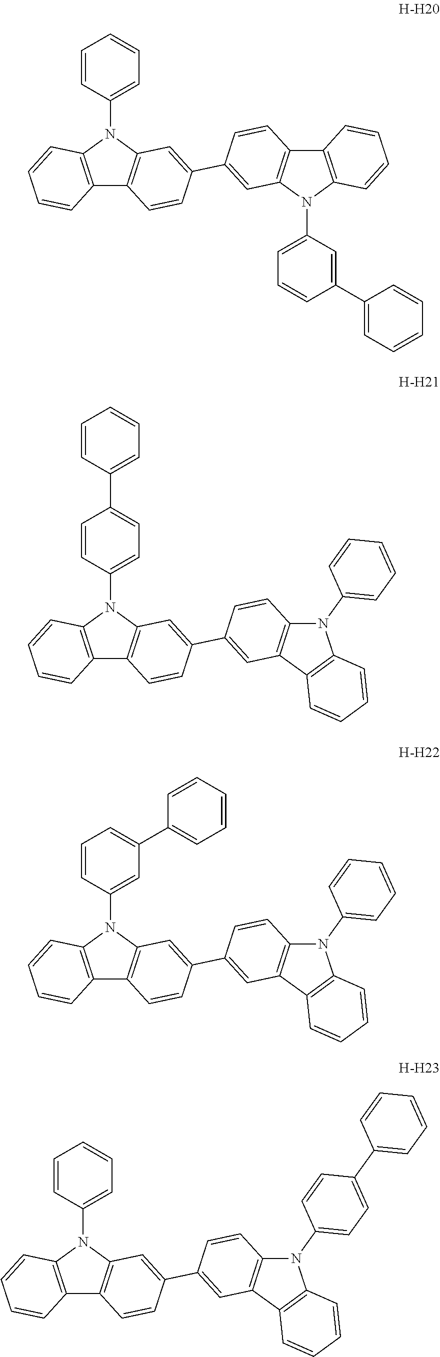 [SMILES: c1ccc(-c2cccc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c(c5)c5ccccc5n6-c5ccccc5)cc43)cc2)cc1 ; c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1]

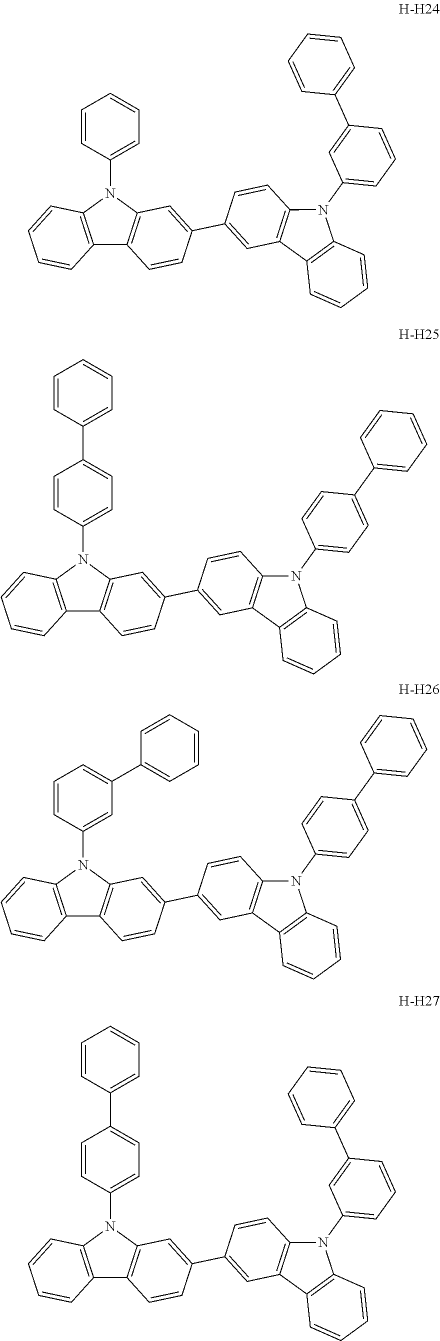 [SMILES: c1ccc(-c2cccc(-n3c4ccccc4c4cc(-c5ccc6c7ccccc7n(-c7ccccc7)c6c5)ccc43)c2)cc1 ; c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1]

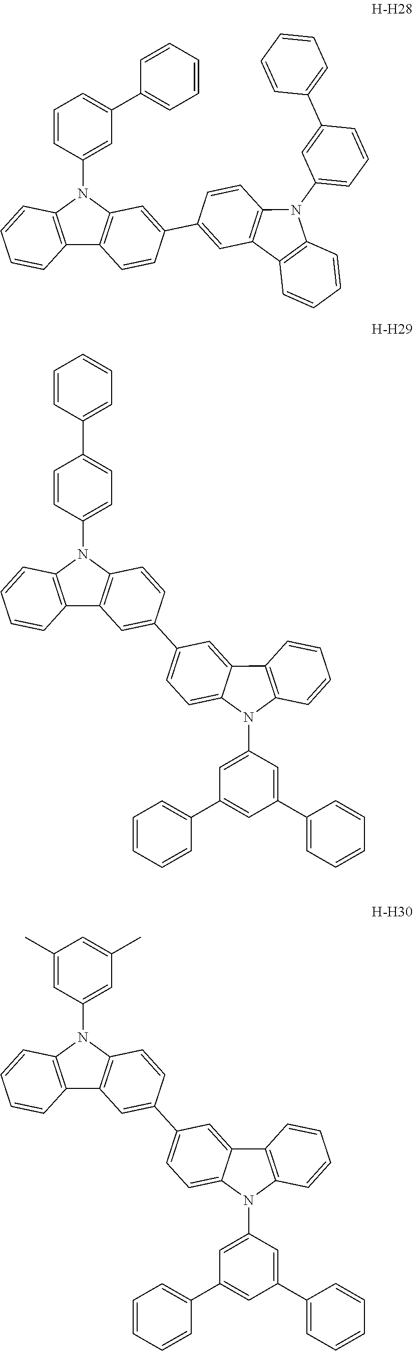 [SMILES: c1ccc(-c2cccc(-n3c4ccccc4c4cc(-c5ccc6c7ccccc7n(-c7cccc(-c8ccccc8)c7)c6c5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; Cc1cc(C)cc(-n2c3ccccc3c3cc(-c4ccc5c(c4)c4ccccc4n5-c4cc(-c5ccccc5)cc(-c5ccccc5)c4)ccc32)c1]

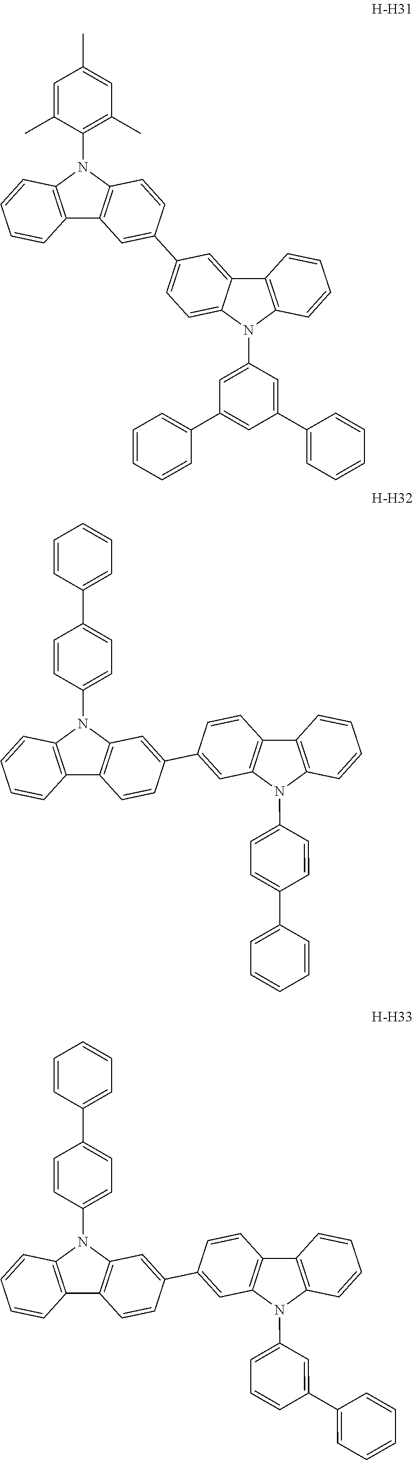 [SMILES: Cc1cc(C)c(-n2c3ccccc3c3cc(-c4ccc5c(c4)c4ccccc4n5-c4cc(-c5ccccc5)cc(-c5ccccc5)c4)ccc32)c(C)c1 ; c1ccc(-c2ccc(-n3c4ccccc4c4ccc(-c5ccc6c7ccccc7n(-c7ccc(-c8ccccc8)cc7)c6c5)cc43)cc2)cc1 ; c1ccc(-c2ccc(-n3c4ccccc4c4ccc(-c5ccc6c7ccccc7n(-c7cccc(-c8ccccc8)c7)c6c5)cc43)cc2)cc1]

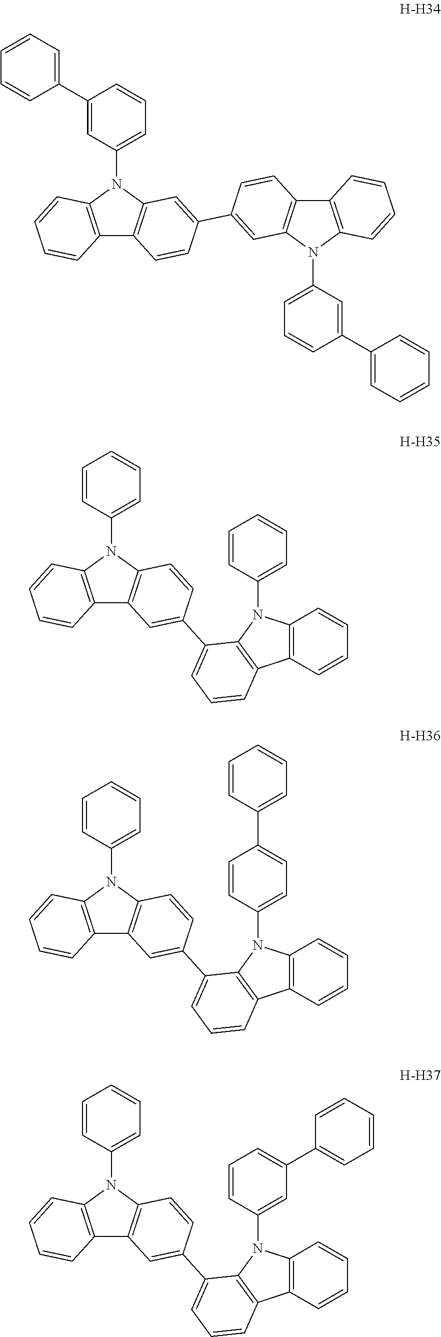 [SMILES: c1ccc(-c2cccc(-n3c4ccccc4c4ccc(-c5ccc6c7ccccc7n(-c7cccc(-c8ccccc8)c7)c6c5)cc43)c2)cc1 ; c1ccc(-n2c3ccccc3c3cc(-c4cccc5c6ccccc6n(-c6ccccc6)c45)ccc32)cc1 ; c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1]

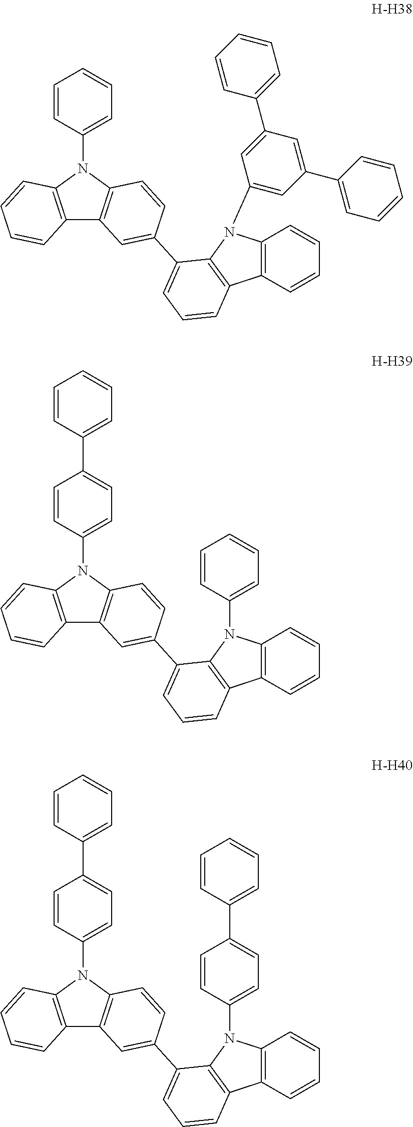 [SMILES: c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2ccc(-n3c4ccccc4c4cc(-c5cccc6c7ccccc7n(-c7ccccc7)c56)ccc43)cc2)cc1 ; c1ccc(-c2ccc(-n3c4ccccc4c4cc(-c5cccc6c7ccccc7n(-c7ccc(-c8ccccc8)cc7)c56)ccc43)cc2)cc1]

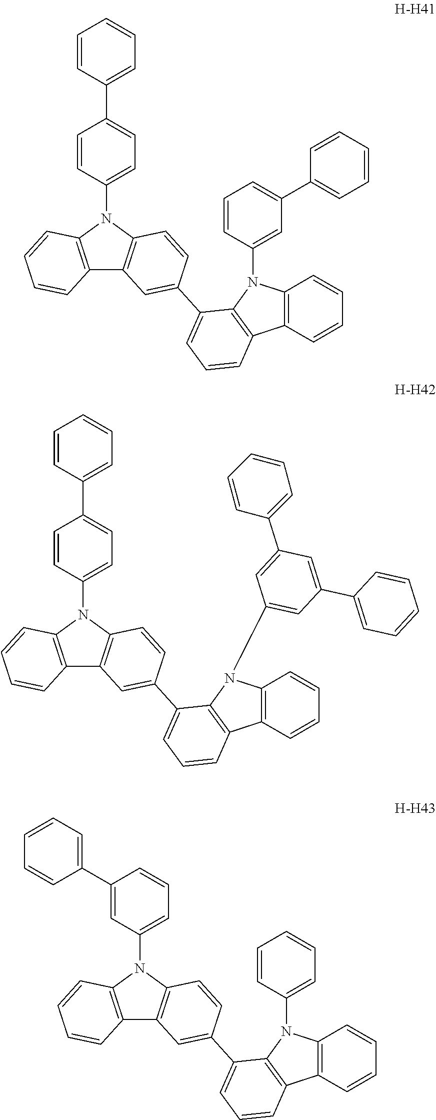 [SMILES: c1ccc(-c2ccc(-n3c4ccccc4c4cc(-c5cccc6c7ccccc7n(-c7cccc(-c8ccccc8)c7)c56)ccc43)cc2)cc1 ; c1ccc(-c2ccc(-n3c4ccccc4c4cc(-c5cccc6c7ccccc7n(-c7cc(-c8ccccc8)cc(-c8ccccc8)c7)c56)ccc43)cc2)cc1 ; c1ccc(-c2cccc(-n3c4ccccc4c4cc(-c5cccc6c7ccccc7n(-c7ccccc7)c56)ccc43)c2)cc1]

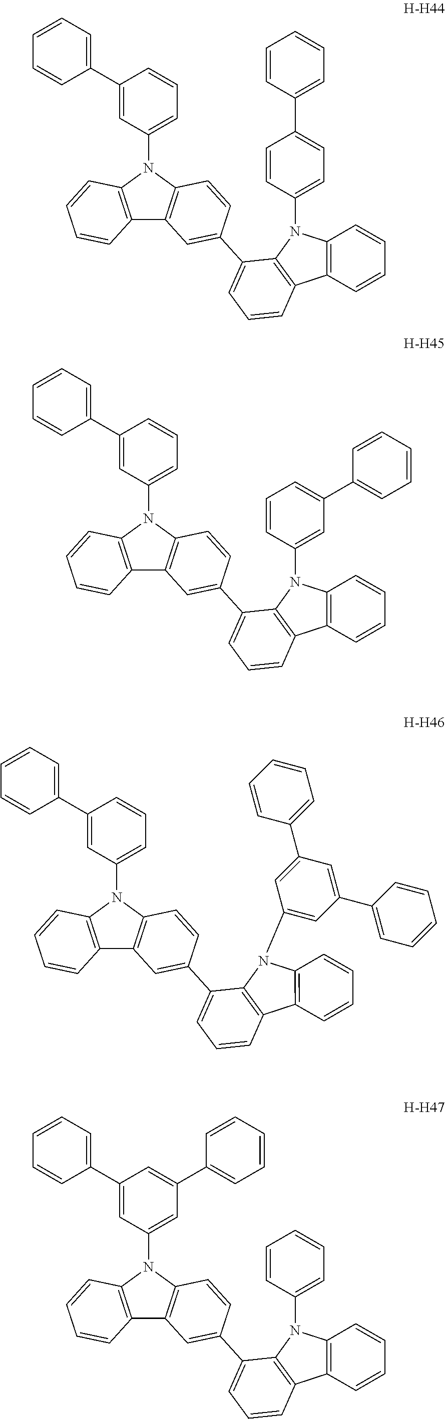 [SMILES: c1ccc(-c2ccc(-n3c4ccccc4c4cccc(-c5ccc6c(c5)c5ccccc5n6-c5cccc(-c6ccccc6)c5)c43)cc2)cc1 ; c1ccc(-c2cccc(-n3c4ccccc4c4cc(-c5cccc6c7ccccc7n(-c7cccc(-c8ccccc8)c7)c56)ccc43)c2)cc1 ; c1ccc(-c2cccc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4cc(-c5cccc6c7ccccc7n(-c7ccccc7)c56)ccc43)c2)cc1]

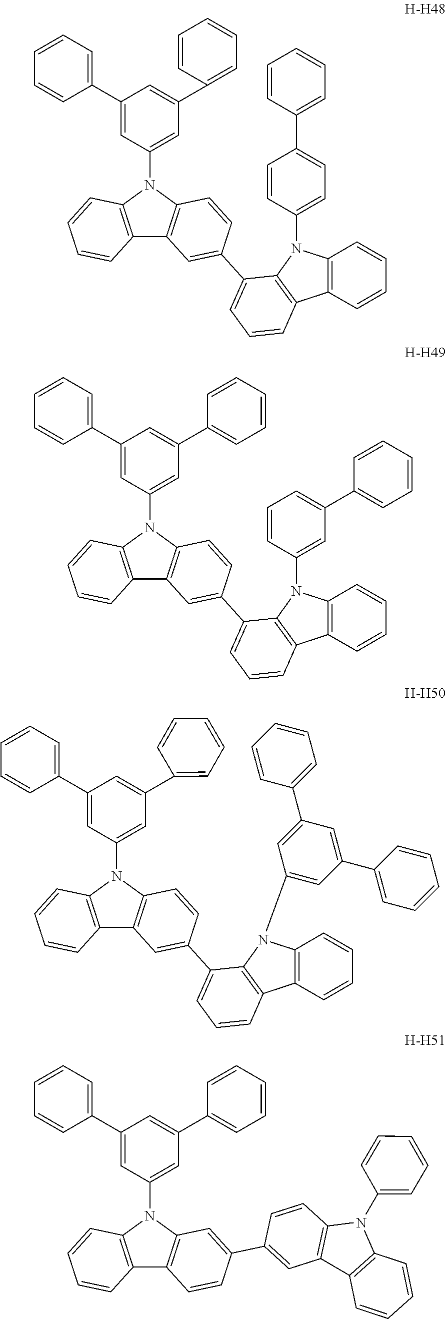

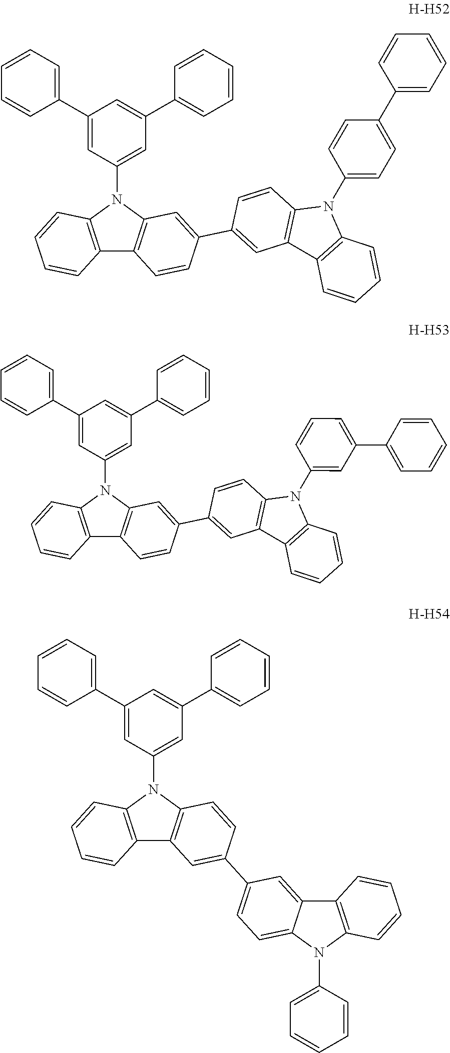 [SMILES: c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

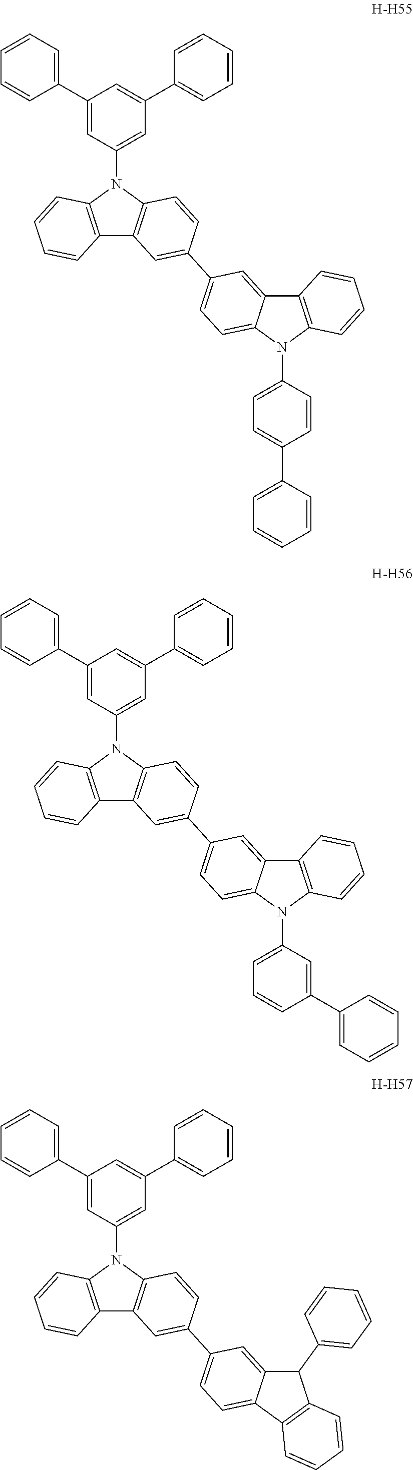 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)C(c5ccccc5)c5ccccc5-6)ccc43)c2)cc1]

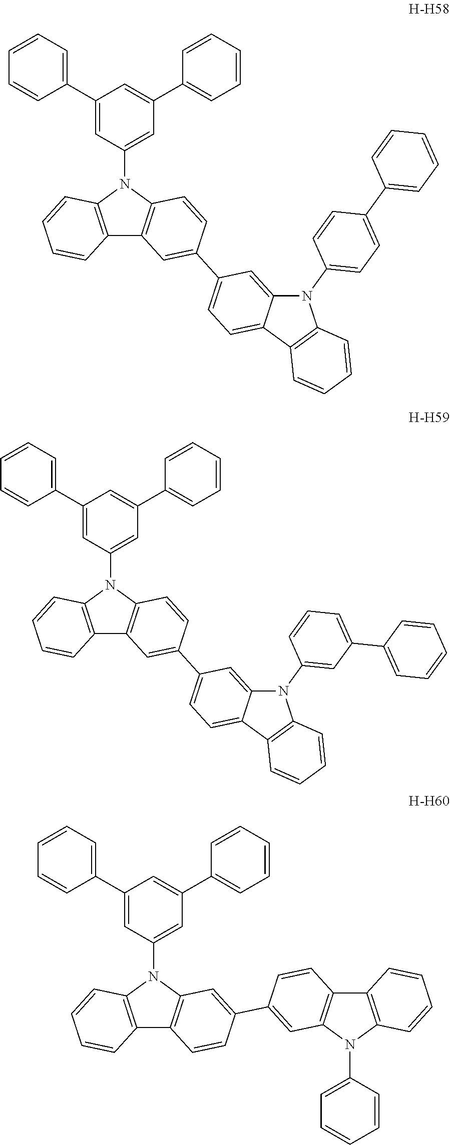 [SMILES: c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1 ; c1ccc(-c2cc(-c3ccccc3)cc(-n3c4ccccc4c4ccc(-c5ccc6c7ccccc7n(-c7ccccc7)c6c5)cc43)c2)cc1]

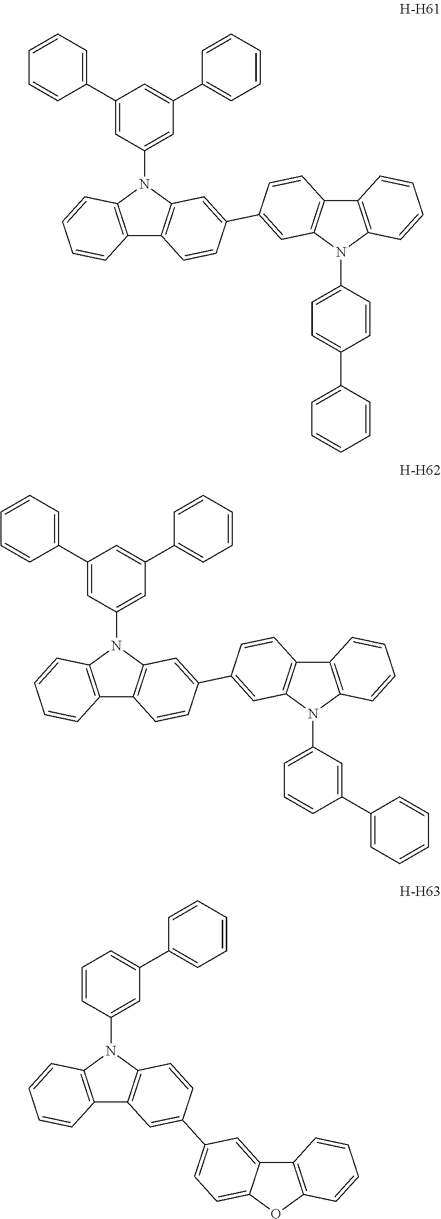 [SMILES: c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1 ; c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-c2cccc(-n3c4ccccc4c4cc(-c5ccc6oc7ccccc7c6c5)ccc43)c2)cc1]

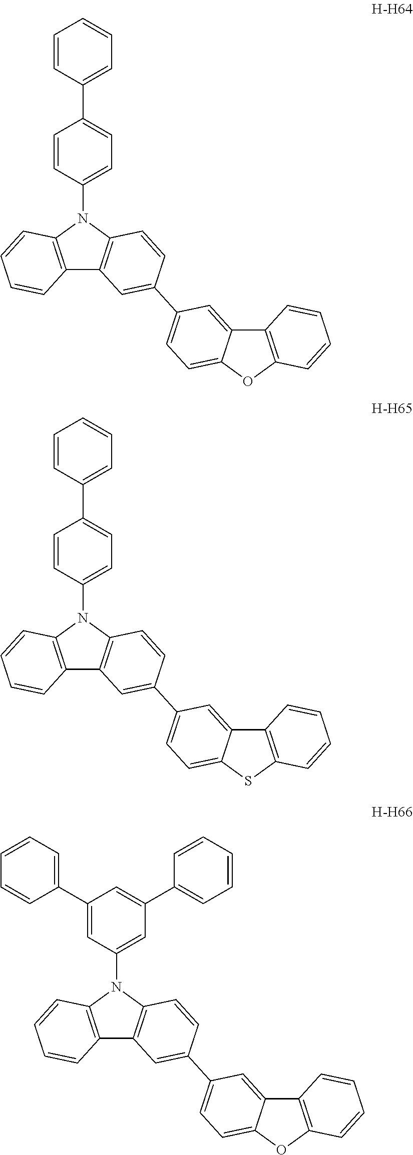 [SMILES: c1ccc(-c2ccc(-n3c4ccccc4c4cc(-c5ccc6oc7ccccc7c6c5)ccc43)cc2)cc1 ; c1ccc(-c2ccc(-n3c4ccccc4c4cc(-c5ccc6sc7ccccc7c6c5)ccc43)cc2)cc1 ; c1ccc(-c2cc(-c3ccccc3)cc(-n3c4ccccc4c4cc(-c5ccc6oc7ccccc7c6c5)ccc43)c2)cc1]

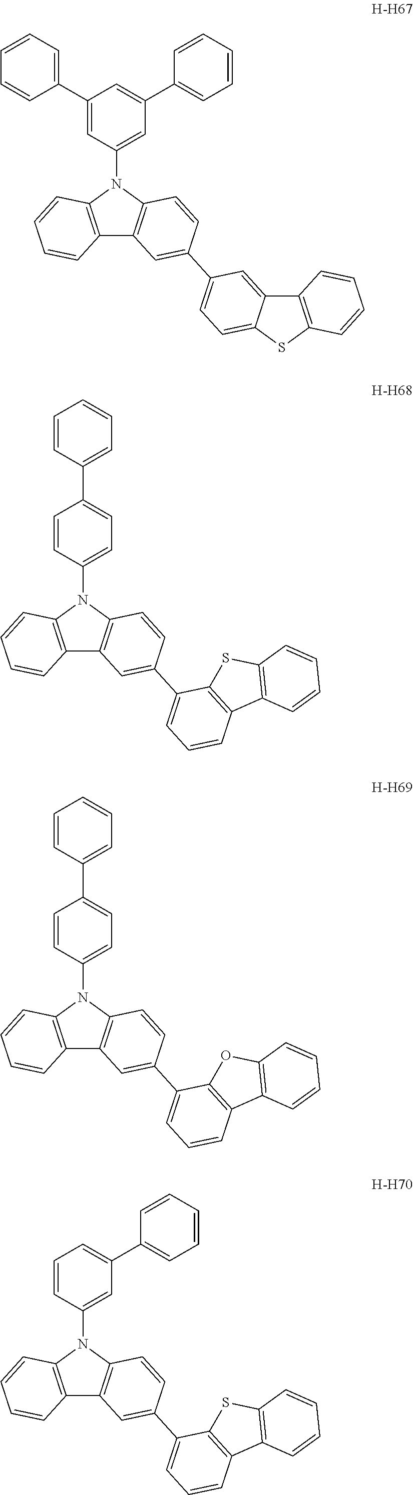 [SMILES: c1ccc(-c2cc(-c3ccccc3)cc(-n3c4ccccc4c4cc(-c5ccc6sc7ccccc7c6c5)ccc43)c2)cc1 ; c1ccc(-c2ccc(-n3c4ccccc4c4cc(-c5cccc6c5sc5ccccc56)ccc43)cc2)cc1 ; c1ccc(-c2ccc(-n3c4ccccc4c4cc(-c5cccc6c5oc5ccccc56)ccc43)cc2)cc1 ; c1ccc(-c2cccc(-n3c4ccccc4c4cc(-c5cccc6c5sc5ccccc56)ccc43)c2)cc1]

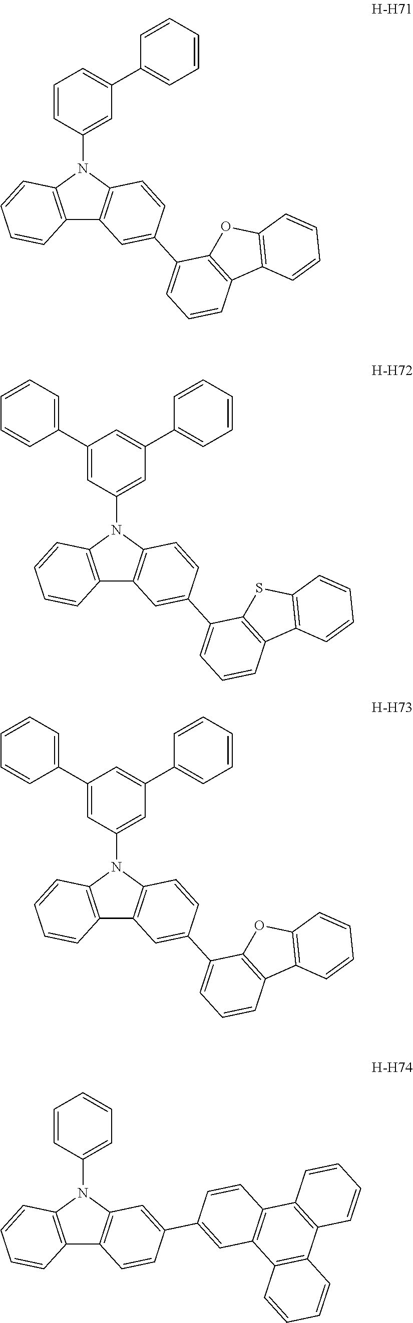

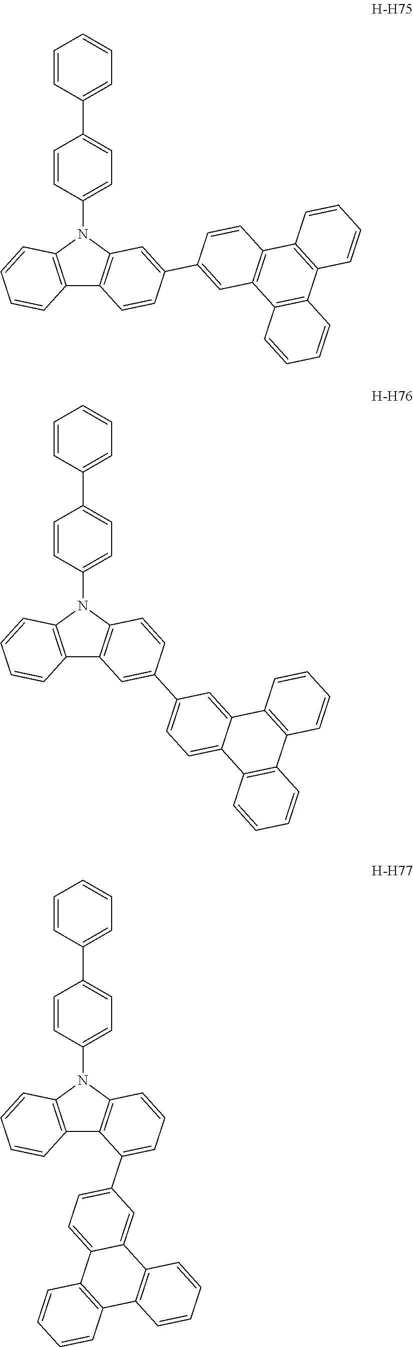 [SMILES: c1ccc(-c2ccc(-n3c4ccccc4c4ccc(-c5ccc6c7ccccc7c7ccccc7c6c5)cc43)cc2)cc1 ; c1ccc(-c2ccc(-n3c4ccccc4c4cc(-c5ccc6c7ccccc7c7ccccc7c6c5)ccc43)cc2)cc1 ; c1ccc(-c2ccc(-n3c4ccccc4c4c(-c5ccc6c7ccccc7c7ccccc7c6c5)cccc43)cc2)cc1]

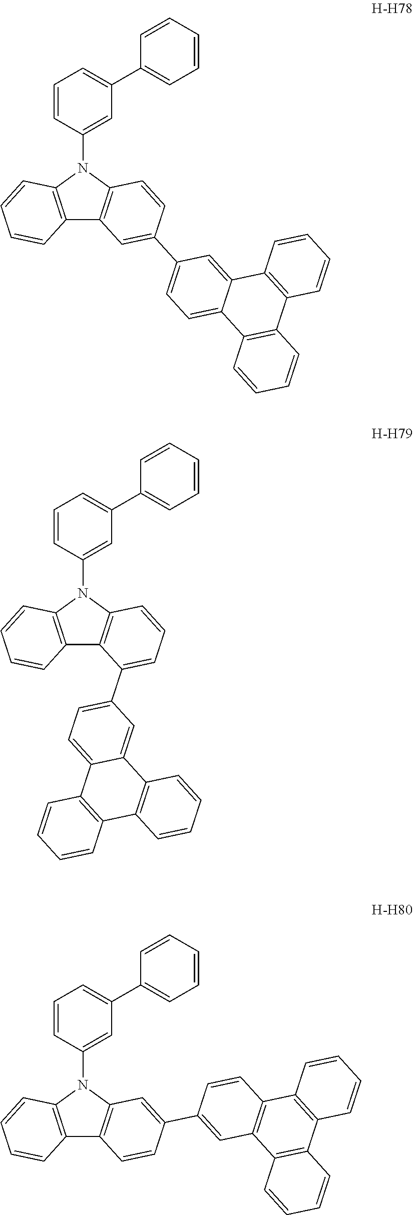 [SMILES: c1ccc(-c2cccc(-n3c4ccccc4c4cc(-c5ccc6c7ccccc7c7ccccc7c6c5)ccc43)c2)cc1 ; c1ccc(-c2cccc(-n3c4ccccc4c4c(-c5ccc6c7ccccc7c7ccccc7c6c5)cccc43)c2)cc1 ; c1ccc(-c2cccc(-n3c4ccccc4c4ccc(-c5ccc6c7ccccc7c7ccccc7c6c5)cc43)c2)cc1]

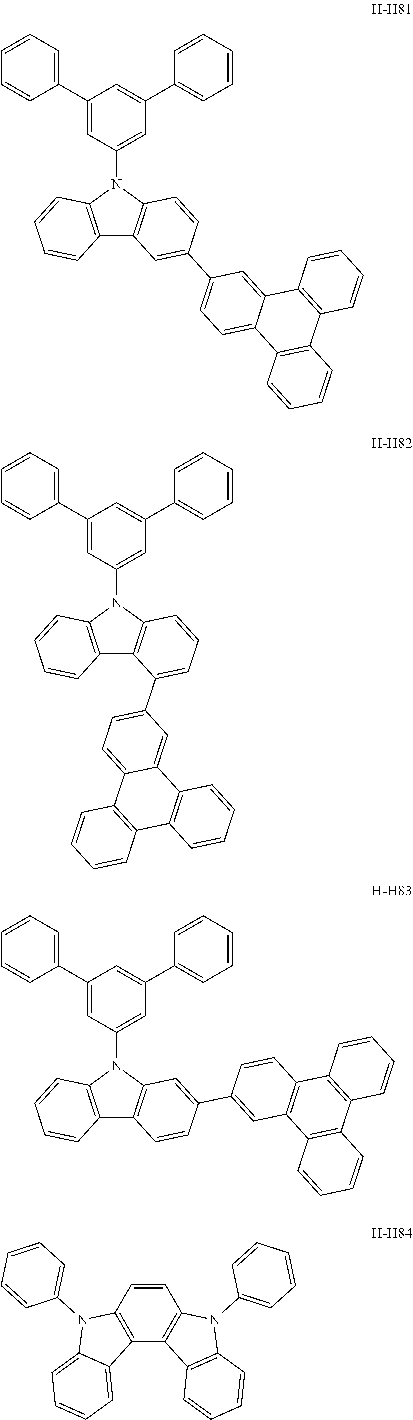 [SMILES: c1ccc(-c2cc(-c3ccccc3)cc(-n3c4ccccc4c4cc(-c5ccc6c7ccccc7c7ccccc7c6c5)ccc43)c2)cc1 ; c1ccc(-c2cc(-c3ccccc3)cc(-n3c4ccccc4c4c(-c5ccc6c7ccccc7c7ccccc7c6c5)cccc43)c2)cc1 ; c1ccc(-c2cc(-c3ccccc3)cc(-n3c4ccccc4c4ccc(-c5ccc6c7ccccc7c7ccccc7c6c5)cc43)c2)cc1 ; c1ccc(-n2c3ccccc3c3c4c5ccccc5n(-c5ccccc5)c4ccc32)cc1]

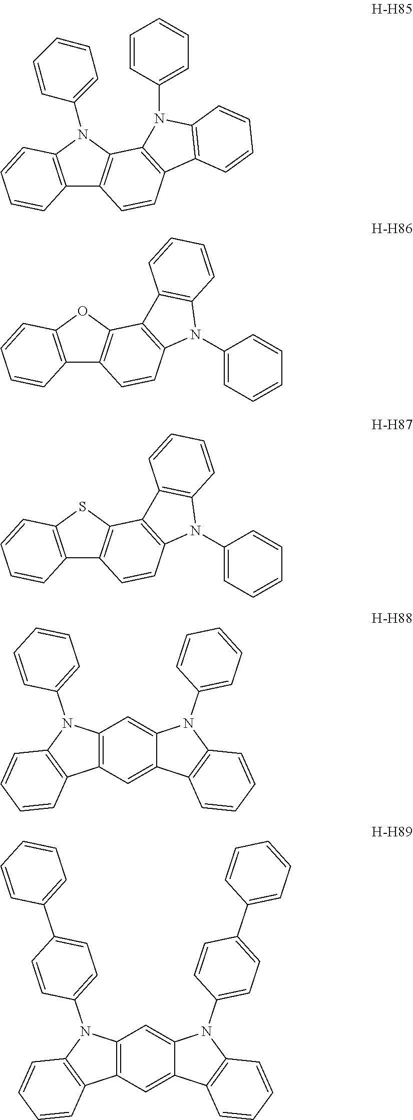

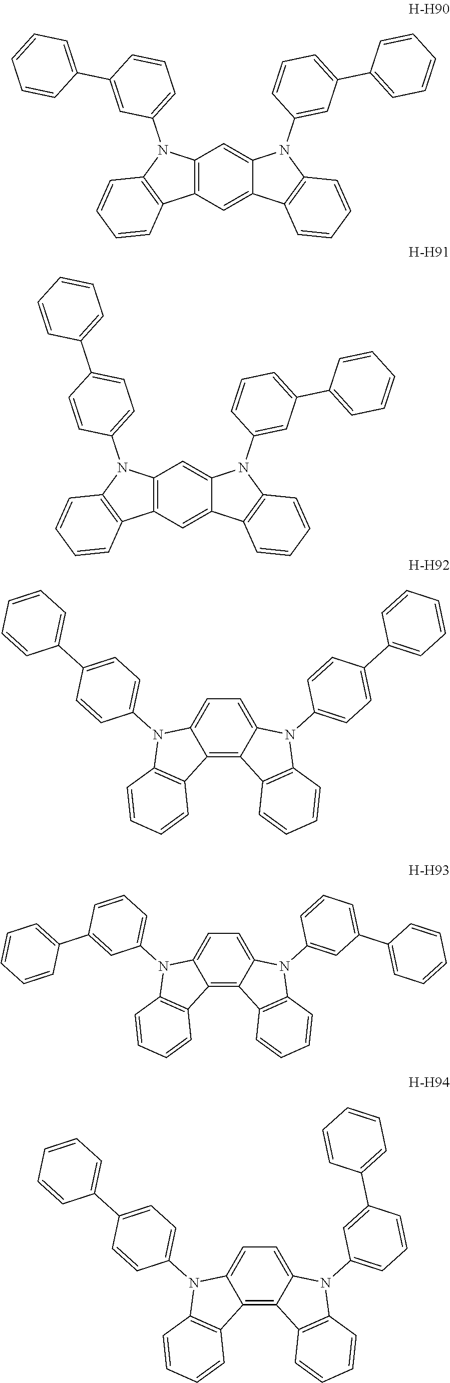 [SMILES: c1ccc(-c2cccc(-n3c4ccccc4c4cc5c6ccccc6n(-c6cccc(-c7ccccc7)c6)c5cc43)c2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2cccc(-n3c4ccccc4c4c5c6ccccc6n(-c6cccc(-c7ccccc7)c6)c5ccc43)c2)cc1 ; c1ccc(-c2ccc(-n3c4ccccc4c4c5c6ccccc6n(-c6cccc(-c7ccccc7)c6)c5ccc43)cc2)cc1]

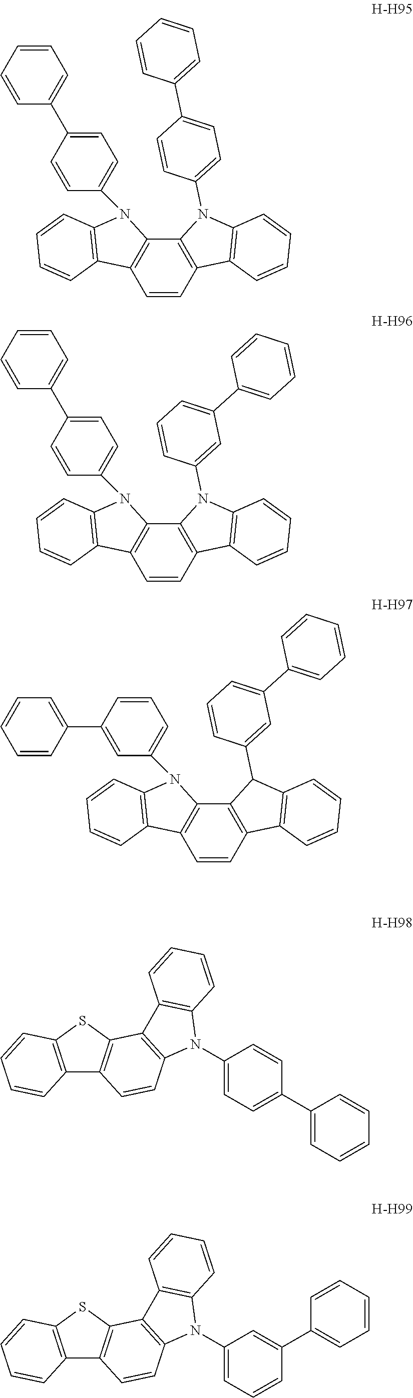 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(C3c4ccccc4-c4ccc5c6ccccc6n(-c6cccc(-c7ccccc7)c6)c5c43)c2)cc1 ; c1ccc(-c2ccc(-n3c4ccccc4c4c5sc6ccccc6c5ccc43)cc2)cc1 ; c1ccc(-c2cccc(-n3c4ccccc4c4c5sc6ccccc6c5ccc43)c2)cc1]

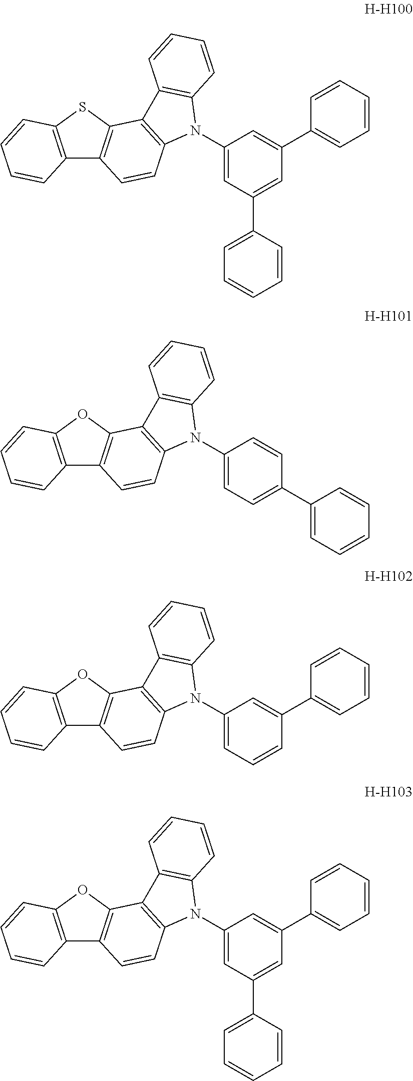 [SMILES: c1ccc(-c2cc(-c3ccccc3)cc(-n3c4ccccc4c4c5sc6ccccc6c5ccc43)c2)cc1 ; c1ccc(-c2ccc(-n3c4ccccc4c4c5oc6ccccc6c5ccc43)cc2)cc1 ; c1ccc(-c2cccc(-n3c4ccccc4c4c5oc6ccccc6c5ccc43)c2)cc1 ; c1ccc(-c2cc(-c3ccccc3)cc(-n3c4ccccc4c4c5oc6ccccc6c5ccc43)c2)cc1]

In some embodiments, the bipolar host may be a compound of Group HEH1, but embodiments are not limited thereto:

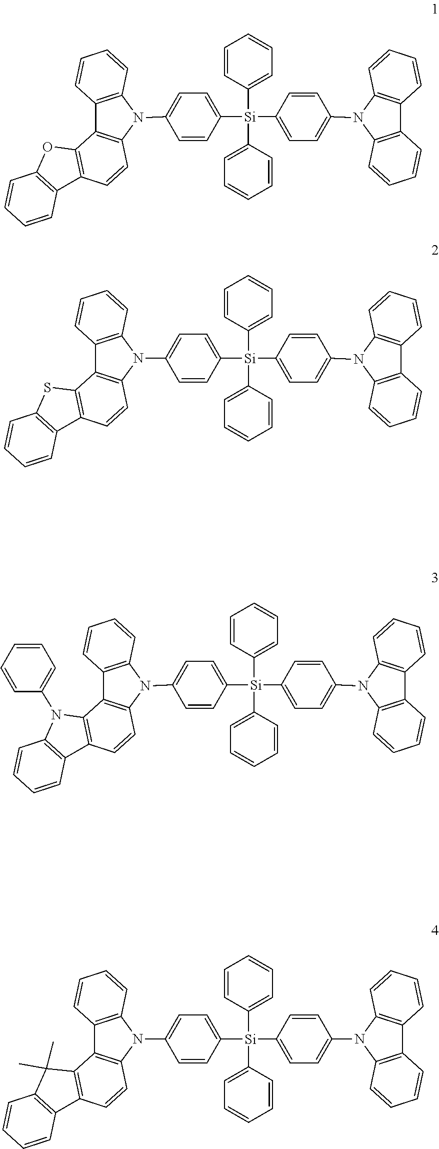 [SMILES: c1ccc([Si](c2ccccc2)(c2ccc(-n3c4ccccc4c4ccccc43)cc2)c2ccc(-n3c4ccccc4c4c5oc6ccccc6c5ccc43)cc2)cc1 ; c1ccc([Si](c2ccccc2)(c2ccc(-n3c4ccccc4c4ccccc43)cc2)c2ccc(-n3c4ccccc4c4c5sc6ccccc6c5ccc43)cc2)cc1 ; c1ccc(-n2c3ccccc3c3ccc4c(c5ccccc5n4-c4ccc([Si](c5ccccc5)(c5ccccc5)c5ccc(-n6c7ccccc7c7ccccc76)cc5)cc4)c32)cc1 ; CC1(C)c2ccccc2-c2ccc3c(c21)c1ccccc1n3-c1ccc([Si](c2ccccc2)(c2ccccc2)c2ccc(-n3c4ccccc4c4ccccc43)cc2)cc1]

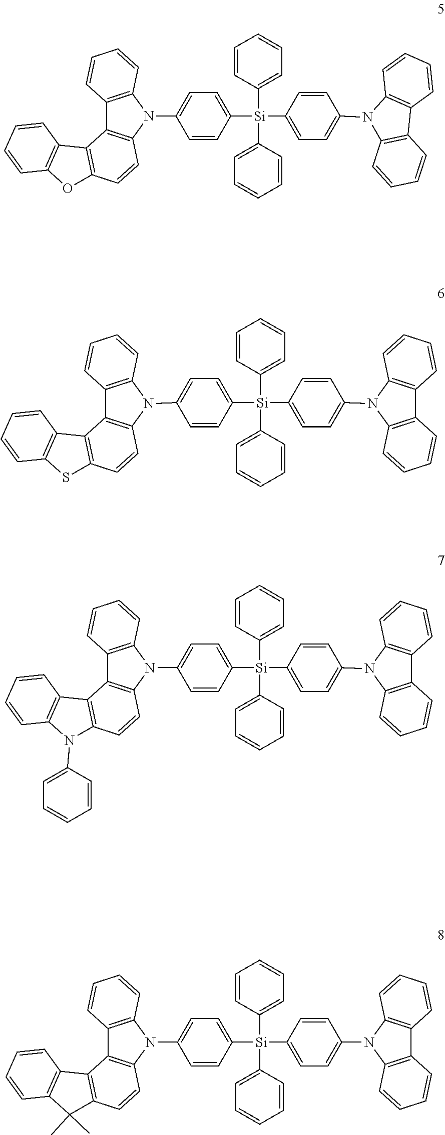 [SMILES: c1ccc([Si](c2ccccc2)(c2ccc(-n3c4ccccc4c4ccccc43)cc2)c2ccc(-n3c4ccccc4c4c5c(ccc43)oc3ccccc35)cc2)cc1 ; c1ccc([Si](c2ccccc2)(c2ccc(-n3c4ccccc4c4ccccc43)cc2)c2ccc(-n3c4ccccc4c4c5c(ccc43)sc3ccccc35)cc2)cc1 ; c1ccc(-n2c3ccccc3c3c4c5ccccc5n(-c5ccc([Si](c6ccccc6)(c6ccccc6)c6ccc(-n7c8ccccc8c8ccccc87)cc6)cc5)c4ccc32)cc1 ; CC1(C)c2ccccc2-c2c1ccc1c2c2ccccc2n1-c1ccc([Si](c2ccccc2)(c2ccccc2)c2ccc(-n3c4ccccc4c4ccccc43)cc2)cc1]

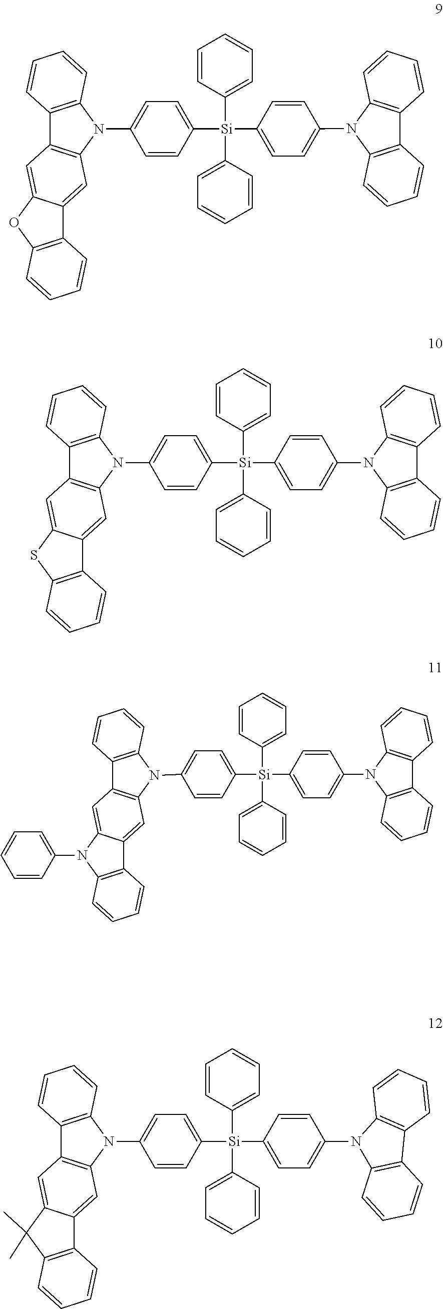 [SMILES: c1ccc([Si](c2ccccc2)(c2ccc(-n3c4ccccc4c4ccccc43)cc2)c2ccc(-n3c4ccccc4c4cc5oc6ccccc6c5cc43)cc2)cc1 ; c1ccc([Si](c2ccccc2)(c2ccc(-n3c4ccccc4c4ccccc43)cc2)c2ccc(-n3c4ccccc4c4cc5sc6ccccc6c5cc43)cc2)cc1 ; c1ccc(-n2c3ccccc3c3cc4c(cc32)c2ccccc2n4-c2ccc([Si](c3ccccc3)(c3ccccc3)c3ccc(-n4c5ccccc5c5ccccc54)cc3)cc2)cc1 ; CC1(C)c2ccccc2-c2cc3c(cc21)c1ccccc1n3-c1ccc([Si](c2ccccc2)(c2ccccc2)c2ccc(-n3c4ccccc4c4ccccc43)cc2)cc1]

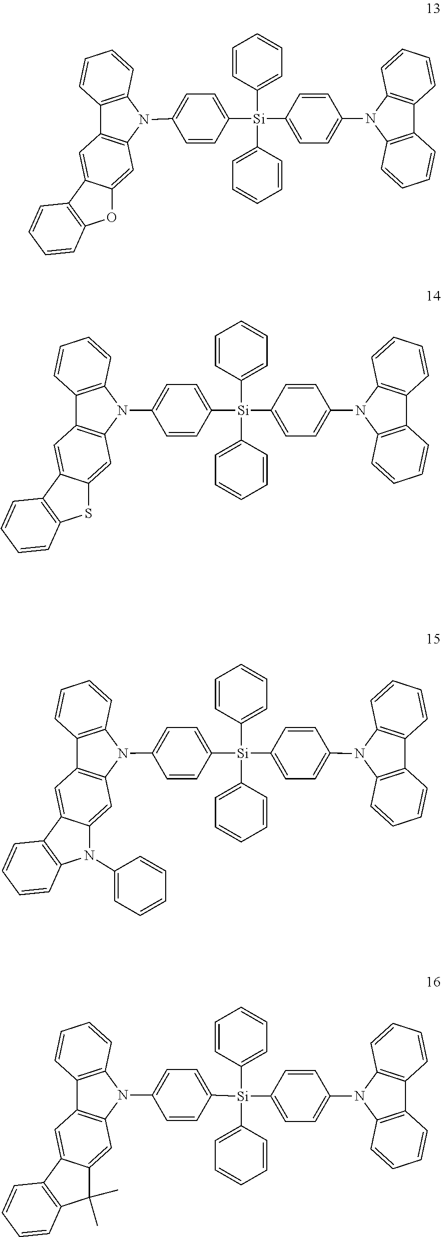 [SMILES: c1ccc([Si](c2ccccc2)(c2ccc(-n3c4ccccc4c4ccccc43)cc2)c2ccc(-n3c4ccccc4c4cc5c(cc43)oc3ccccc35)cc2)cc1 ; c1ccc([Si](c2ccccc2)(c2ccc(-n3c4ccccc4c4ccccc43)cc2)c2ccc(-n3c4ccccc4c4cc5c(cc43)sc3ccccc35)cc2)cc1 ; c1ccc(-n2c3ccccc3c3cc4c5ccccc5n(-c5ccc([Si](c6ccccc6)(c6ccccc6)c6ccc(-n7c8ccccc8c8ccccc87)cc6)cc5)c4cc32)cc1 ; CC1(C)c2ccccc2-c2cc3c4ccccc4n(-c4ccc([Si](c5ccccc5)(c5ccccc5)c5ccc(-n6c7ccccc7c7ccccc76)cc5)cc4)c3cc21]

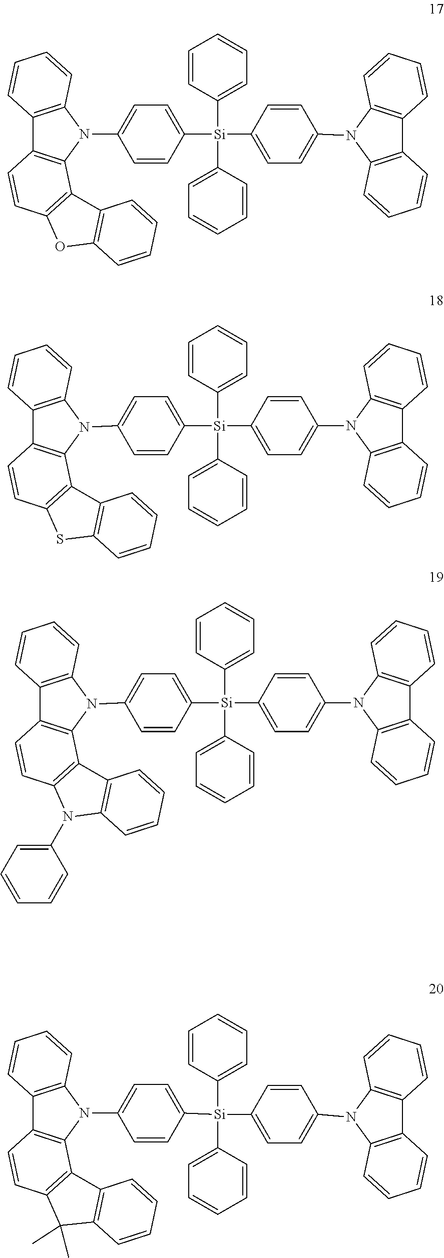 [SMILES: c1ccc([Si](c2ccccc2)(c2ccc(-n3c4ccccc4c4ccccc43)cc2)c2ccc(-n3c4ccccc4c4ccc5oc6ccccc6c5c43)cc2)cc1 ; c1ccc([Si](c2ccccc2)(c2ccc(-n3c4ccccc4c4ccccc43)cc2)c2ccc(-n3c4ccccc4c4ccc5sc6ccccc6c5c43)cc2)cc1 ; c1ccc(-n2c3ccccc3c3c2ccc2c4ccccc4n(-c4ccc([Si](c5ccccc5)(c5ccccc5)c5ccc(-n6c7ccccc7c7ccccc76)cc5)cc4)c23)cc1 ; CC1(C)c2ccccc2-c2c1ccc1c3ccccc3n(-c3ccc([Si](c4ccccc4)(c4ccccc4)c4ccc(-n5c6ccccc6c6ccccc65)cc4)cc3)c21]

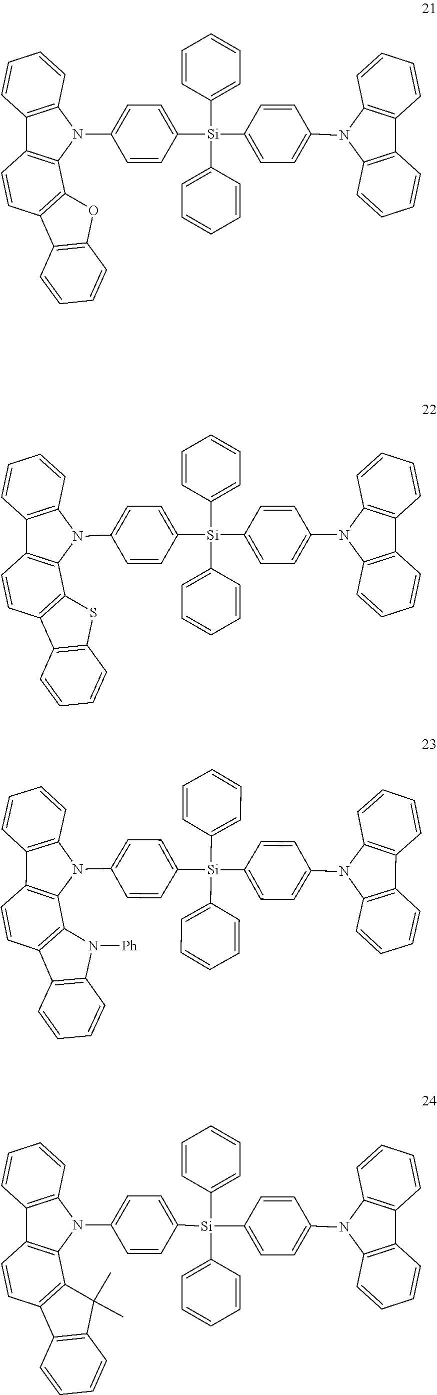 [SMILES: c1ccc([Si](c2ccccc2)(c2ccc(-n3c4ccccc4c4ccccc43)cc2)c2ccc(-n3c4ccccc4c4ccc5c6ccccc6oc5c43)cc2)cc1 ; c1ccc([Si](c2ccccc2)(c2ccc(-n3c4ccccc4c4ccccc43)cc2)c2ccc(-n3c4ccccc4c4ccc5c6ccccc6sc5c43)cc2)cc1 ; c1ccc(-n2c3ccccc3c3ccc4c5ccccc5n(-c5ccc([Si](c6ccccc6)(c6ccccc6)c6ccc(-n7c8ccccc8c8ccccc87)cc6)cc5)c4c32)cc1 ; CC1(C)c2ccccc2-c2ccc3c4ccccc4n(-c4ccc([Si](c5ccccc5)(c5ccccc5)c5ccc(-n6c7ccccc7c7ccccc76)cc5)cc4)c3c21]

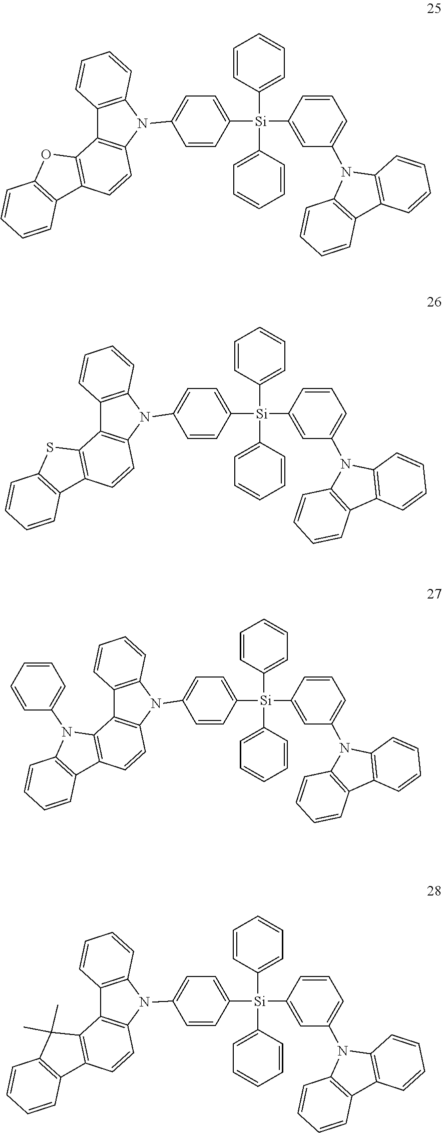 [SMILES: c1ccc([Si](c2ccccc2)(c2ccc(-n3c4ccccc4c4c5oc6ccccc6c5ccc43)cc2)c2cccc(-n3c4ccccc4c4ccccc43)c2)cc1 ; c1ccc([Si](c2ccccc2)(c2ccc(-n3c4ccccc4c4c5sc6ccccc6c5ccc43)cc2)c2cccc(-n3c4ccccc4c4ccccc43)c2)cc1 ; c1ccc(-n2c3ccccc3c3ccc4c(c5ccccc5n4-c4ccc([Si](c5ccccc5)(c5ccccc5)c5cccc(-n6c7ccccc7c7ccccc76)c5)cc4)c32)cc1 ; CC1(C)c2ccccc2-c2ccc3c(c21)c1ccccc1n3-c1ccc([Si](c2ccccc2)(c2ccccc2)c2cccc(-n3c4ccccc4c4ccccc43)c2)cc1]

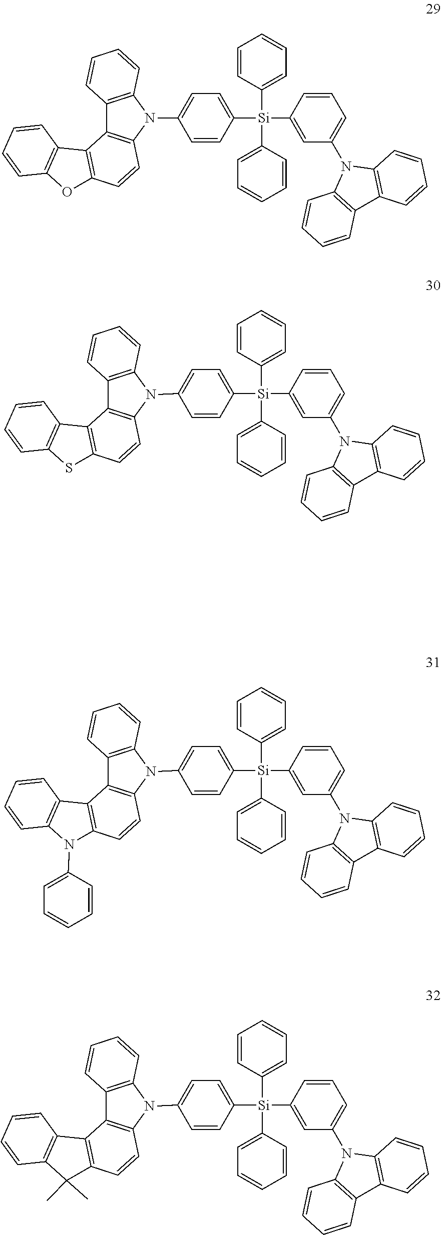 [SMILES: c1ccc([Si](c2ccccc2)(c2ccc(-n3c4ccccc4c4c5c(ccc43)oc3ccccc35)cc2)c2cccc(-n3c4ccccc4c4ccccc43)c2)cc1 ; c1ccc([Si](c2ccccc2)(c2ccc(-n3c4ccccc4c4c5c(ccc43)sc3ccccc35)cc2)c2cccc(-n3c4ccccc4c4ccccc43)c2)cc1 ; c1ccc(-n2c3ccccc3c3c4c5ccccc5n(-c5ccc([Si](c6ccccc6)(c6ccccc6)c6cccc(-n7c8ccccc8c8ccccc87)c6)cc5)c4ccc32)cc1 ; CC1(C)c2ccccc2-c2c1ccc1c2c2ccccc2n1-c1ccc([Si](c2ccccc2)(c2ccccc2)c2cccc(-n3c4ccccc4c4ccccc43)c2)cc1]

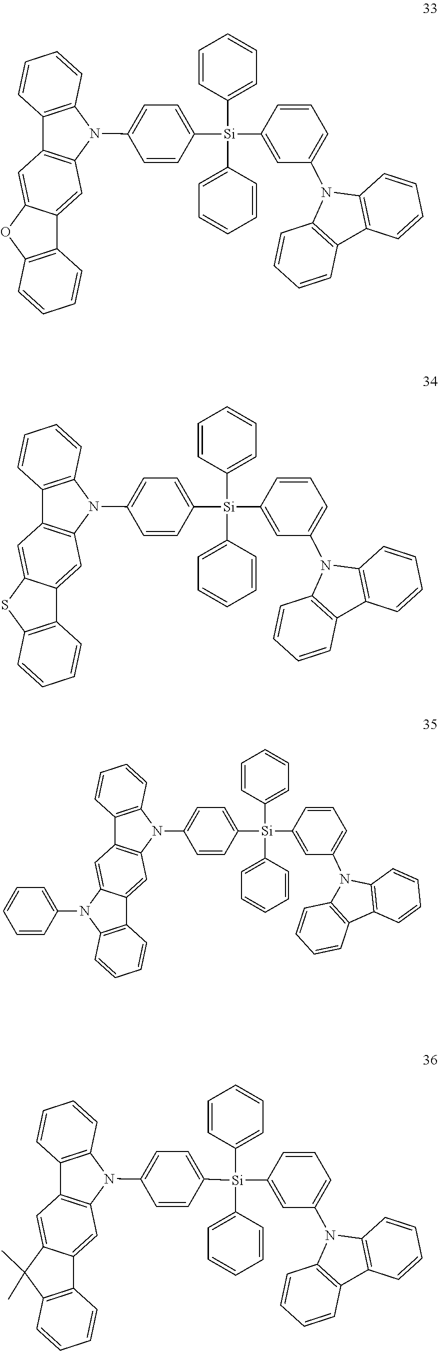 [SMILES: c1ccc([Si](c2ccccc2)(c2ccc(-n3c4ccccc4c4cc5oc6ccccc6c5cc43)cc2)c2cccc(-n3c4ccccc4c4ccccc43)c2)cc1 ; c1ccc([Si](c2ccccc2)(c2ccc(-n3c4ccccc4c4cc5sc6ccccc6c5cc43)cc2)c2cccc(-n3c4ccccc4c4ccccc43)c2)cc1 ; c1ccc(-n2c3ccccc3c3cc4c(cc32)c2ccccc2n4-c2ccc([Si](c3ccccc3)(c3ccccc3)c3cccc(-n4c5ccccc5c5ccccc54)c3)cc2)cc1 ; CC1(C)c2ccccc2-c2cc3c(cc21)c1ccccc1n3-c1ccc([Si](c2ccccc2)(c2ccccc2)c2cccc(-n3c4ccccc4c4ccccc43)c2)cc1]

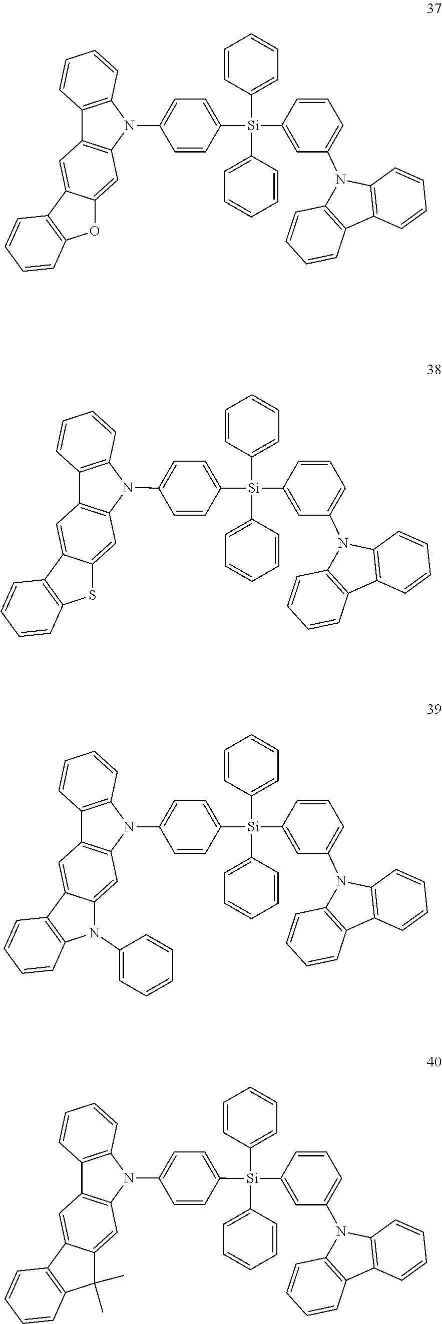 [SMILES: c1ccc([Si](c2ccccc2)(c2ccc(-n3c4ccccc4c4cc5c(cc43)oc3ccccc35)cc2)c2cccc(-n3c4ccccc4c4ccccc43)c2)cc1 ; c1ccc([Si](c2ccccc2)(c2ccc(-n3c4ccccc4c4cc5c(cc43)sc3ccccc35)cc2)c2cccc(-n3c4ccccc4c4ccccc43)c2)cc1 ; c1ccc(-n2c3ccccc3c3cc4c5ccccc5n(-c5ccc([Si](c6ccccc6)(c6ccccc6)c6cccc(-n7c8ccccc8c8ccccc87)c6)cc5)c4cc32)cc1 ; CC1(C)c2ccccc2-c2cc3c4ccccc4n(-c4ccc([Si](c5ccccc5)(c5ccccc5)c5cccc(-n6c7ccccc7c7ccccc76)c5)cc4)c3cc21]

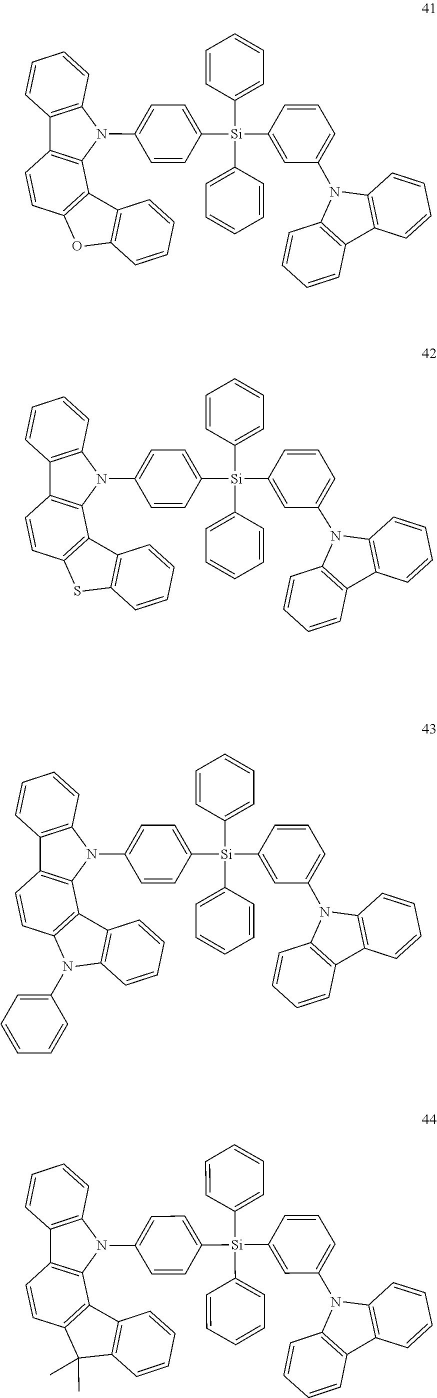 [SMILES: c1ccc([Si](c2ccccc2)(c2ccc(-n3c4ccccc4c4ccc5oc6ccccc6c5c43)cc2)c2cccc(-n3c4ccccc4c4ccccc43)c2)cc1 ; c1ccc([Si](c2ccccc2)(c2ccc(-n3c4ccccc4c4ccc5sc6ccccc6c5c43)cc2)c2cccc(-n3c4ccccc4c4ccccc43)c2)cc1 ; c1ccc(-n2c3ccccc3c3c2ccc2c4ccccc4n(-c4ccc([Si](c5ccccc5)(c5ccccc5)c5cccc(-n6c7ccccc7c7ccccc76)c5)cc4)c23)cc1 ; CC1(C)c2ccccc2-c2c1ccc1c3ccccc3n(-c3ccc([Si](c4ccccc4)(c4ccccc4)c4cccc(-n5c6ccccc6c6ccccc65)c4)cc3)c21]

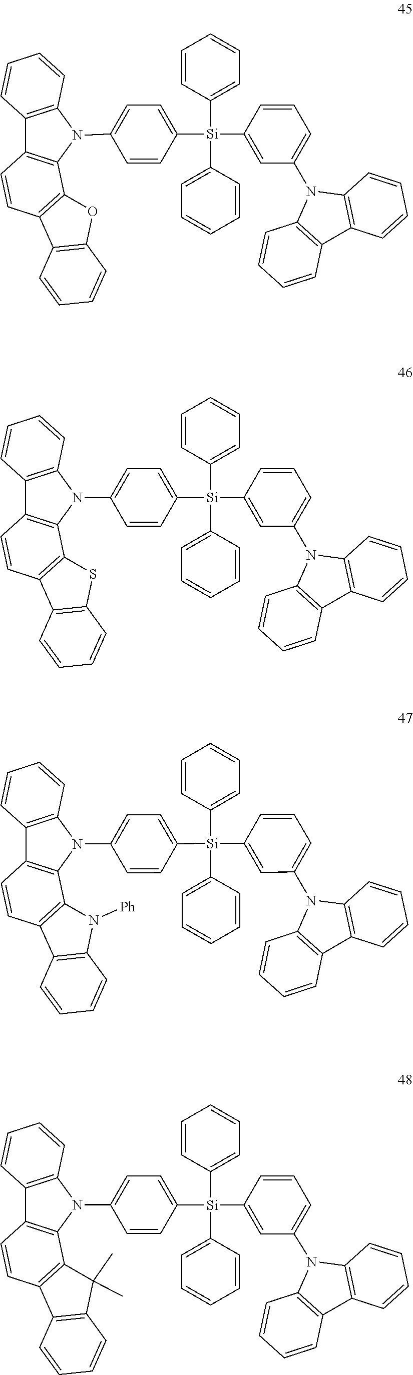 [SMILES: c1ccc([Si](c2ccccc2)(c2ccc(-n3c4ccccc4c4ccc5c6ccccc6oc5c43)cc2)c2cccc(-n3c4ccccc4c4ccccc43)c2)cc1 ; c1ccc([Si](c2ccccc2)(c2ccc(-n3c4ccccc4c4ccc5c6ccccc6sc5c43)cc2)c2cccc(-n3c4ccccc4c4ccccc43)c2)cc1 ; c1ccc(-n2c3ccccc3c3ccc4c5ccccc5n(-c5ccc([Si](c6ccccc6)(c6ccccc6)c6cccc(-n7c8ccccc8c8ccccc87)c6)cc5)c4c32)cc1 ; CC1(C)c2ccccc2-c2ccc3c4ccccc4n(-c4ccc([Si](c5ccccc5)(c5ccccc5)c5cccc(-n6c7ccccc7c7ccccc76)c5)cc4)c3c21]

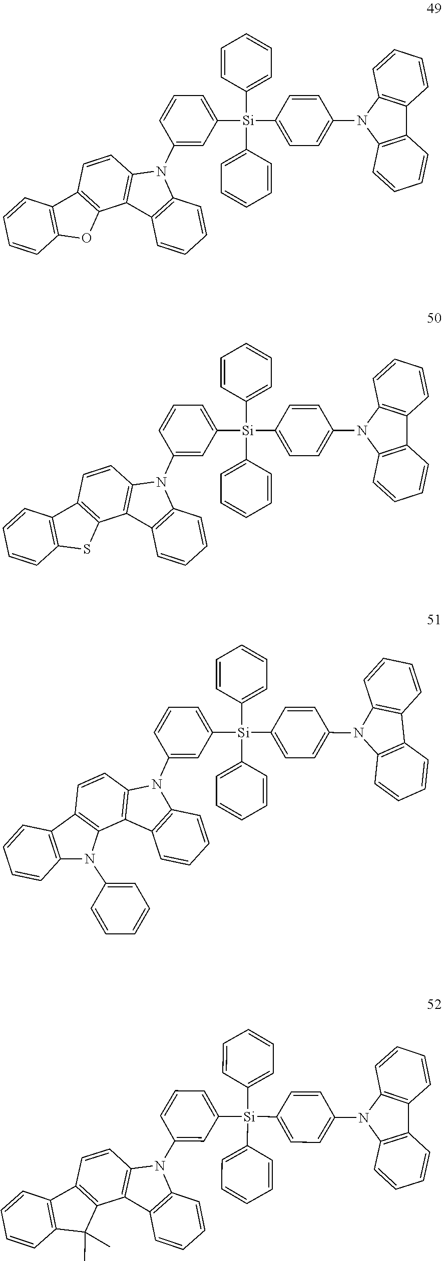 [SMILES: c1ccc([Si](c2ccccc2)(c2ccc(-n3c4ccccc4c4ccccc43)cc2)c2cccc(-n3c4ccccc4c4c5oc6ccccc6c5ccc43)c2)cc1 ; c1ccc([Si](c2ccccc2)(c2ccc(-n3c4ccccc4c4ccccc43)cc2)c2cccc(-n3c4ccccc4c4c5sc6ccccc6c5ccc43)c2)cc1 ; c1ccc(-n2c3ccccc3c3ccc4c(c5ccccc5n4-c4cccc([Si](c5ccccc5)(c5ccccc5)c5ccc(-n6c7ccccc7c7ccccc76)cc5)c4)c32)cc1 ; CC1(C)c2ccccc2-c2ccc3c(c21)c1ccccc1n3-c1cccc([Si](c2ccccc2)(c2ccccc2)c2ccc(-n3c4ccccc4c4ccccc43)cc2)c1]

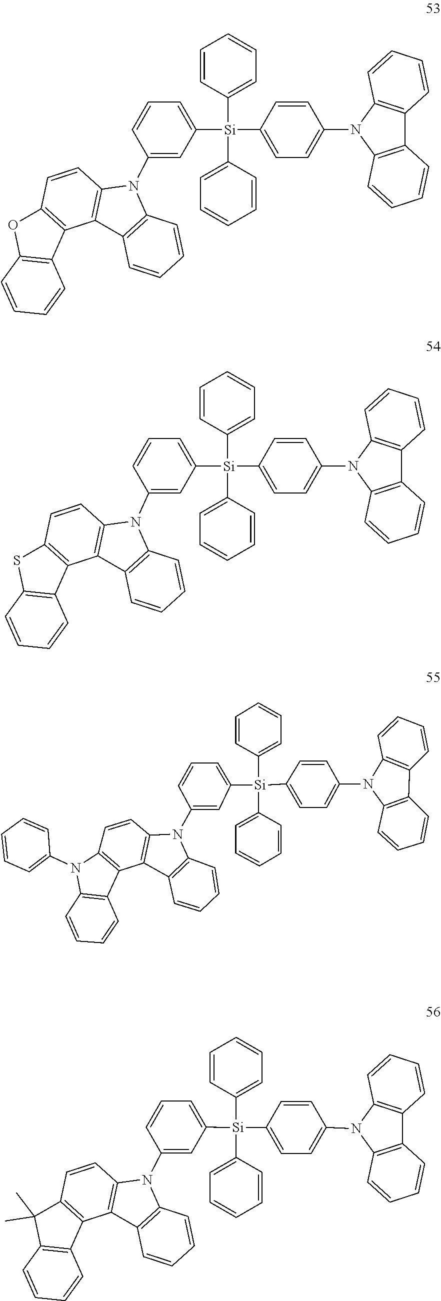 [SMILES: c1ccc([Si](c2ccccc2)(c2ccc(-n3c4ccccc4c4ccccc43)cc2)c2cccc(-n3c4ccccc4c4c5c(ccc43)oc3ccccc35)c2)cc1 ; c1ccc([Si](c2ccccc2)(c2ccc(-n3c4ccccc4c4ccccc43)cc2)c2cccc(-n3c4ccccc4c4c5c(ccc43)sc3ccccc35)c2)cc1 ; C1=CC=C([Si](c2ccccc2)(c2ccc(-n3c4ccccc4c4ccccc43)cc2)c2cccc(-n3c4ccccc4c4c5c6ccccc6n(-c6ccccc6)c5ccc43)c2)CC=C1 ; CC1(C)c2ccccc2-c2c1ccc1c2c2ccccc2n1-c1cccc([Si](c2ccccc2)(c2ccccc2)c2ccc(-n3c4ccccc4c4ccccc43)cc2)c1]

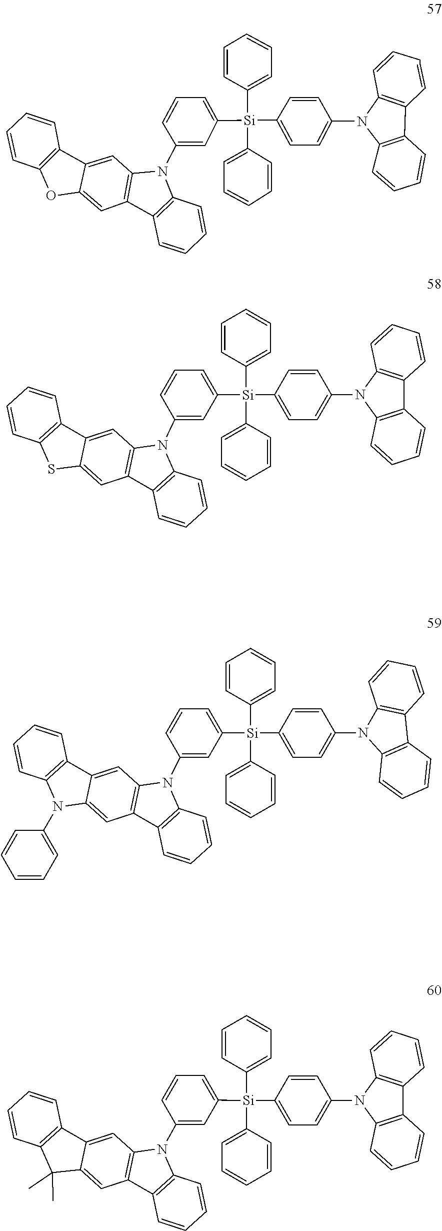 [SMILES: C1=Cc2c(n(-c3ccc([Si](c4ccccc4)(c4ccccc4)c4cccc(-n5c6ccccc6c6cc7oc8ccccc8c7cc65)c4)cc3)c3ccccc23)C=CC1 ; c1ccc([Si](c2ccccc2)(c2ccc(-n3c4ccccc4c4ccccc43)cc2)c2cccc(-n3c4ccccc4c4cc5sc6ccccc6c5cc43)c2)cc1 ; c1ccc(-n2c3ccccc3c3cc4c(cc32)c2ccccc2n4-c2cccc([Si](c3ccccc3)(c3ccccc3)c3ccc(-n4c5ccccc5c5ccccc54)cc3)c2)cc1 ; CC1(C)c2ccccc2-c2cc3c(cc21)c1ccccc1n3-c1cccc([Si](c2ccccc2)(c2ccccc2)c2ccc(-n3c4ccccc4c4ccccc43)cc2)c1]

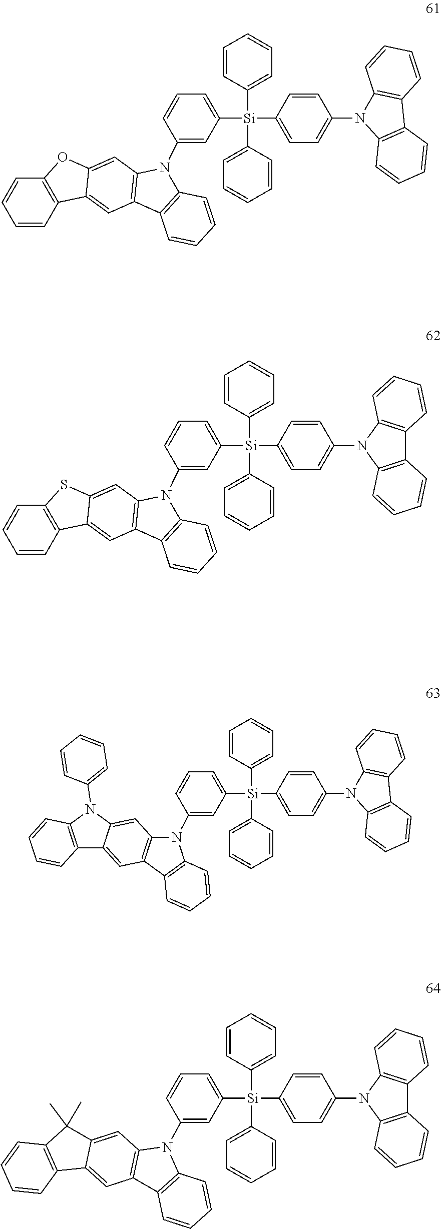 [SMILES: c1ccc([Si](c2ccccc2)(c2ccc(-n3c4ccccc4c4ccccc43)cc2)c2cccc(-n3c4ccccc4c4cc5c(cc43)oc3ccccc35)c2)cc1 ; c1ccc([Si](c2ccccc2)(c2ccc(-n3c4ccccc4c4ccccc43)cc2)c2cccc(-n3c4ccccc4c4cc5c(cc43)sc3ccccc35)c2)cc1 ; c1ccc(-n2c3ccccc3c3cc4c5ccccc5n(-c5cccc([Si](c6ccccc6)(c6ccccc6)c6ccc(-n7c8ccccc8c8ccccc87)cc6)c5)c4cc32)cc1 ; CC1(C)c2ccccc2-c2cc3c4ccccc4n(-c4cccc([Si](c5ccccc5)(c5ccccc5)c5ccc(-n6c7ccccc7c7ccccc76)cc5)c4)c3cc21]

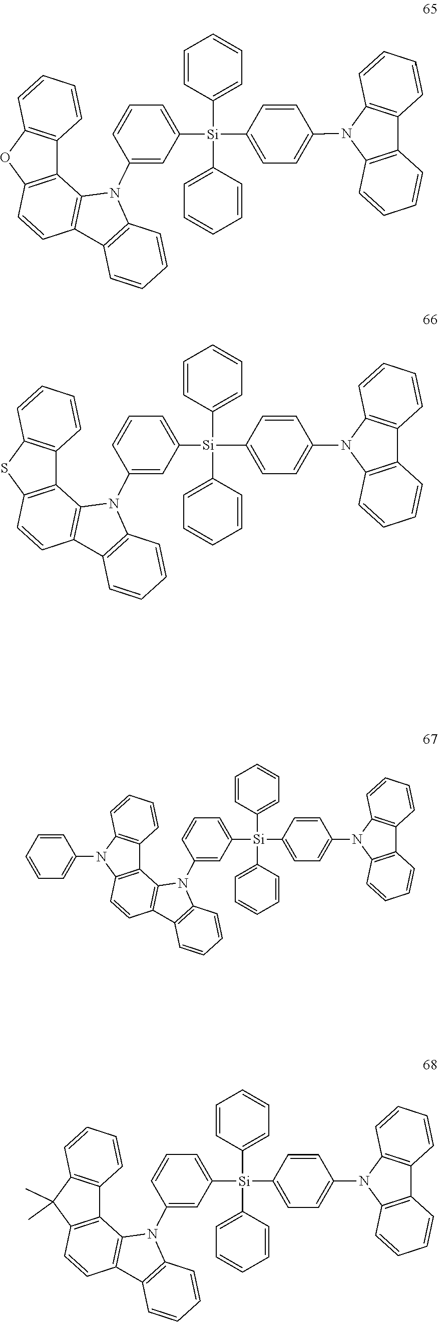

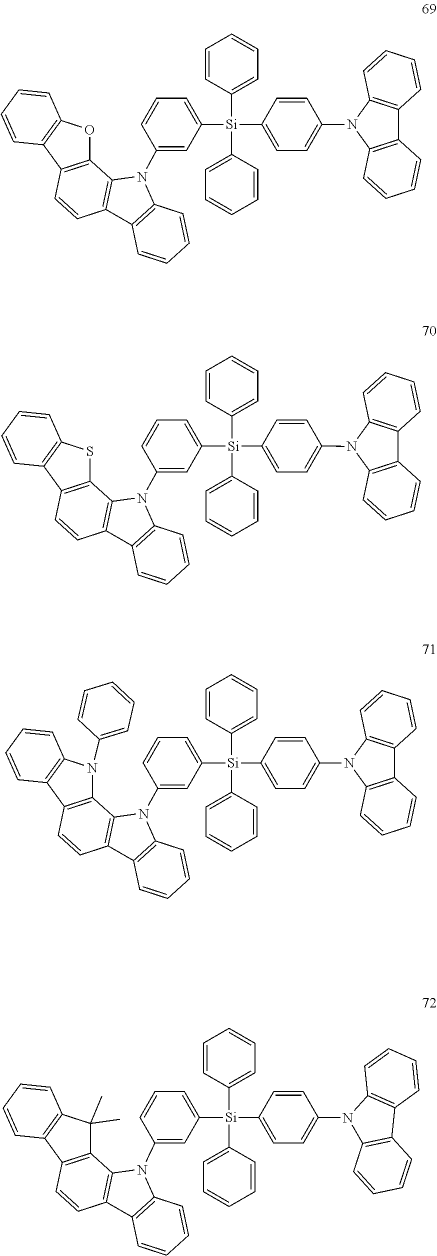 [SMILES: c1ccc([Si](c2ccccc2)(c2ccc(-n3c4ccccc4c4ccccc43)cc2)c2cccc(-n3c4ccccc4c4ccc5c6ccccc6oc5c43)c2)cc1 ; c1ccc([Si](c2ccccc2)(c2ccc(-n3c4ccccc4c4ccccc43)cc2)c2cccc(-n3c4ccccc4c4ccc5c6ccccc6sc5c43)c2)cc1 ; c1ccc(-n2c3ccccc3c3ccc4c5ccccc5n(-c5cccc([Si](c6ccccc6)(c6ccccc6)c6ccc(-n7c8ccccc8c8ccccc87)cc6)c5)c4c32)cc1 ; CC1(C)c2ccccc2-c2ccc3c4ccccc4n(-c4cccc([Si](c5ccccc5)(c5ccccc5)c5ccc(-n6c7ccccc7c7ccccc76)cc5)c4)c3c21]

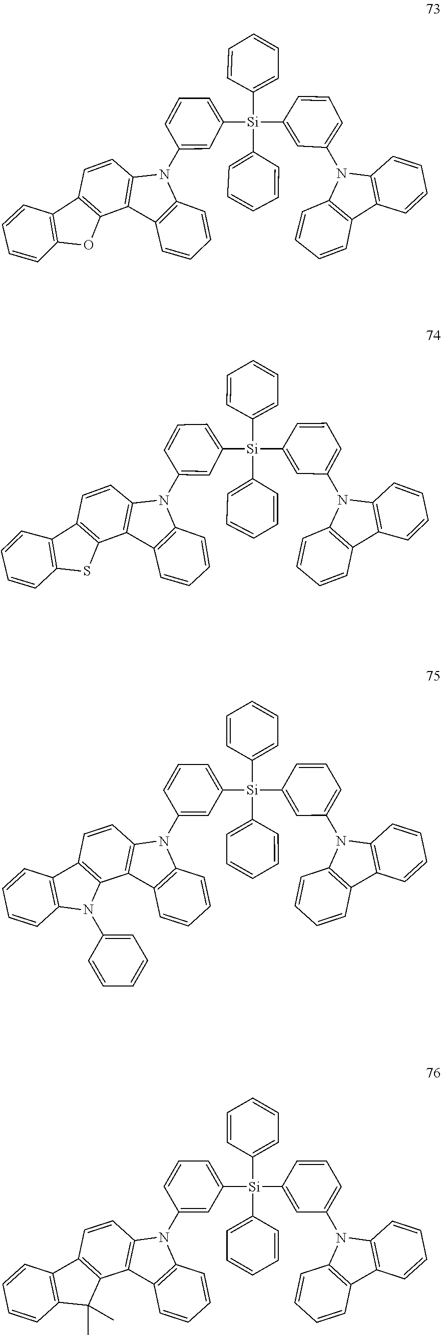 [SMILES: c1ccc([Si](c2ccccc2)(c2cccc(-n3c4ccccc4c4ccccc43)c2)c2cccc(-n3c4ccccc4c4c5oc6ccccc6c5ccc43)c2)cc1 ; c1ccc([Si](c2ccccc2)(c2cccc(-n3c4ccccc4c4ccccc43)c2)c2cccc(-n3c4ccccc4c4c5sc6ccccc6c5ccc43)c2)cc1 ; c1ccc(-n2c3ccccc3c3ccc4c(c5ccccc5n4-c4cccc([Si](c5ccccc5)(c5ccccc5)c5cccc(-n6c7ccccc7c7ccccc76)c5)c4)c32)cc1 ; CC1(C)c2ccccc2-c2ccc3c(c21)c1ccccc1n3-c1cccc([Si](c2ccccc2)(c2ccccc2)c2cccc(-n3c4ccccc4c4ccccc43)c2)c1]

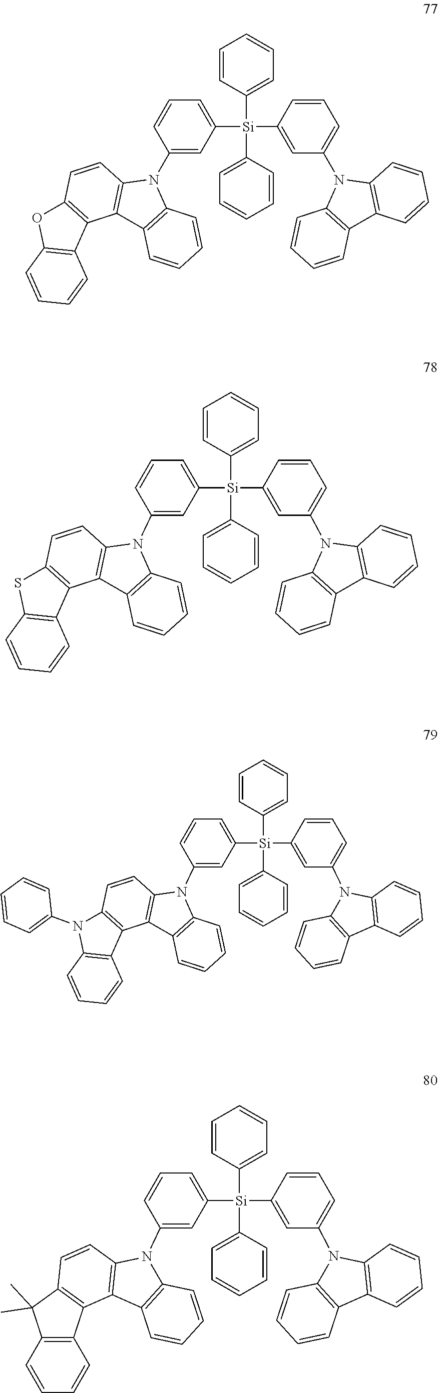 [SMILES: c1ccc([Si](c2ccccc2)(c2cccc(-n3c4ccccc4c4ccccc43)c2)c2cccc(-n3c4ccccc4c4c5c(ccc43)oc3ccccc35)c2)cc1 ; c1ccc([Si](c2ccccc2)(c2cccc(-n3c4ccccc4c4ccccc43)c2)c2cccc(-n3c4ccccc4c4c5c(ccc43)sc3ccccc35)c2)cc1 ; c1ccc(-n2c3ccccc3c3c4c5ccccc5n(-c5cccc([Si](c6ccccc6)(c6ccccc6)c6cccc(-n7c8ccccc8c8ccccc87)c6)c5)c4ccc32)cc1 ; CC1(C)c2ccccc2-c2c1ccc1c2c2ccccc2n1-c1cccc([Si](c2ccccc2)(c2ccccc2)c2cccc(-n3c4ccccc4c4ccccc43)c2)c1]

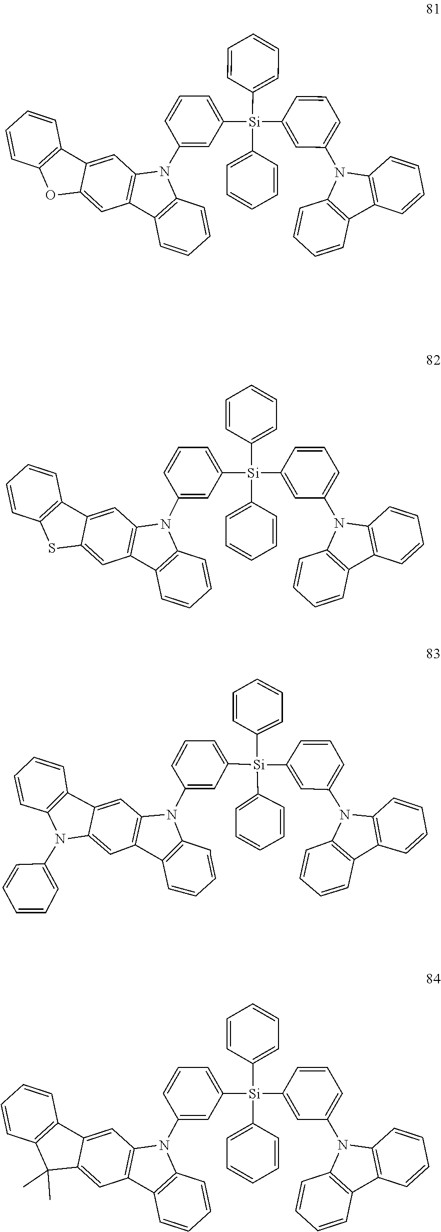 [SMILES: c1ccc([Si](c2ccccc2)(c2cccc(-n3c4ccccc4c4ccccc43)c2)c2cccc(-n3c4ccccc4c4cc5oc6ccccc6c5cc43)c2)cc1 ; c1ccc([Si](c2ccccc2)(c2cccc(-n3c4ccccc4c4ccccc43)c2)c2cccc(-n3c4ccccc4c4cc5sc6ccccc6c5cc43)c2)cc1 ; C1=CCc2c(c3cc4c(cc3n2-c2cccc([Si](c3ccccc3)(c3ccccc3)c3cccc(-n5c6ccccc6c6ccccc65)c3)c2)c2ccccc2n4-c2ccccc2)C=C1 ; CC1(C)c2ccccc2-c2cc3c(cc21)c1ccccc1n3-c1cccc([Si](c2ccccc2)(c2ccccc2)c2cccc(-n3c4ccccc4c4ccccc43)c2)c1]

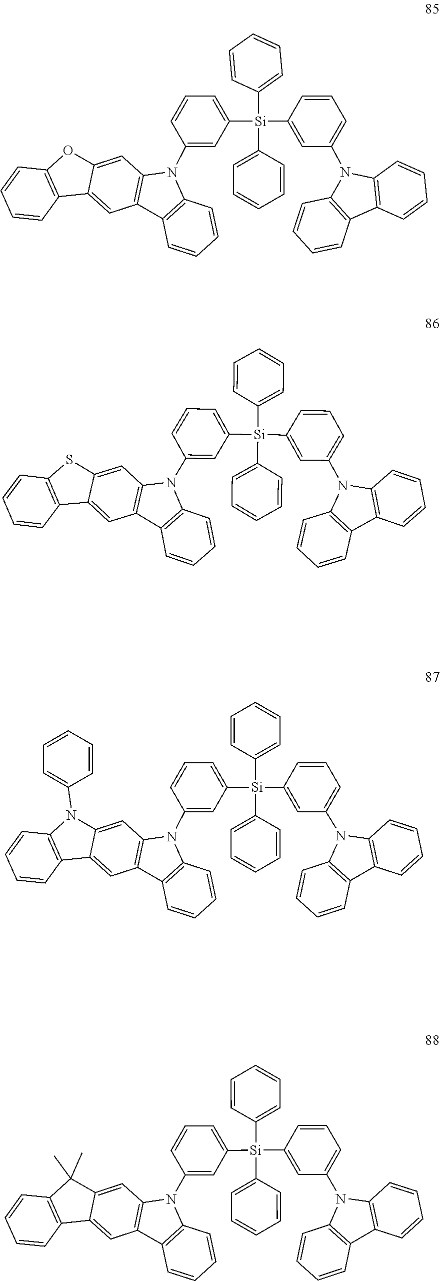 [SMILES: c1ccc([Si](c2ccccc2)(c2cccc(-n3c4ccccc4c4ccccc43)c2)c2cccc(-n3c4ccccc4c4cc5c(cc43)oc3ccccc35)c2)cc1 ; c1ccc([Si](c2ccccc2)(c2cccc(-n3c4ccccc4c4ccccc43)c2)c2cccc(-n3c4ccccc4c4cc5c(cc43)sc3ccccc35)c2)cc1 ; c1ccc(-n2c3ccccc3c3cc4c5ccccc5n(-c5cccc([Si](c6ccccc6)(c6ccccc6)c6cccc(-n7c8ccccc8c8ccccc87)c6)c5)c4cc32)cc1 ; CC1(C)c2ccccc2-c2cc3c4ccccc4n(-c4cccc([Si](c5ccccc5)(c5ccccc5)c5cccc(-n6c7ccccc7c7ccccc76)c5)c4)c3cc21]

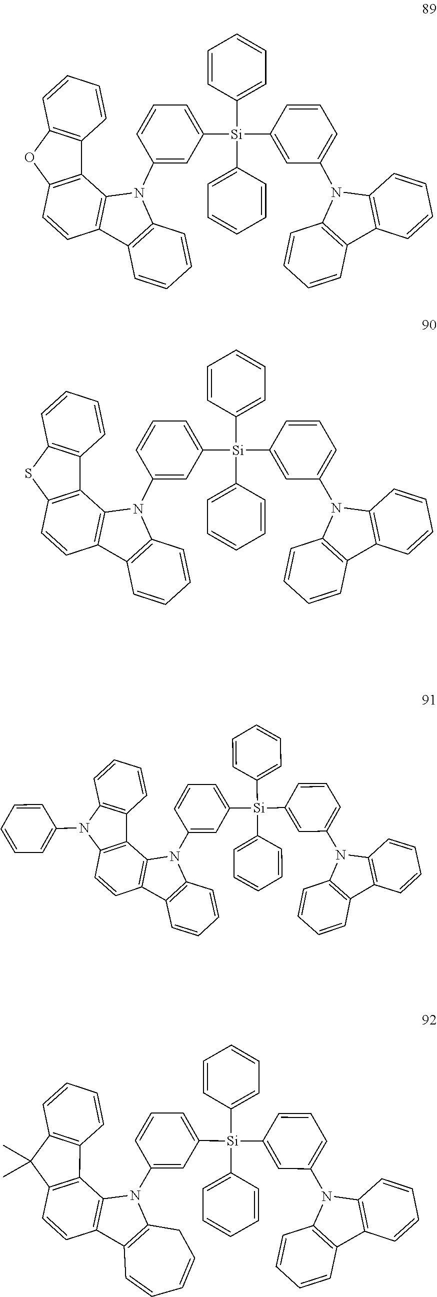 [SMILES: c1ccc([Si](c2ccccc2)(c2cccc(-n3c4ccccc4c4ccccc43)c2)c2cccc(-n3c4ccccc4c4ccc5oc6ccccc6c5c43)c2)cc1 ; c1ccc([Si](c2ccccc2)(c2cccc(-n3c4ccccc4c4ccccc43)c2)c2cccc(-n3c4ccccc4c4ccc5sc6ccccc6c5c43)c2)cc1 ; c1ccc(-n2c3ccccc3c3c2ccc2c4ccccc4n(-c4cccc([Si](c5ccccc5)(c5ccccc5)c5cccc(-n6c7ccccc7c7ccccc76)c5)c4)c23)cc1 ; CC1(C)c2ccccc2-c2c1ccc1c3c(n(-c4cccc([Si](c5ccccc5)(c5ccccc5)c5cccc(-n6c7ccccc7c7ccccc76)c5)c4)c21)CC=CC=C3]

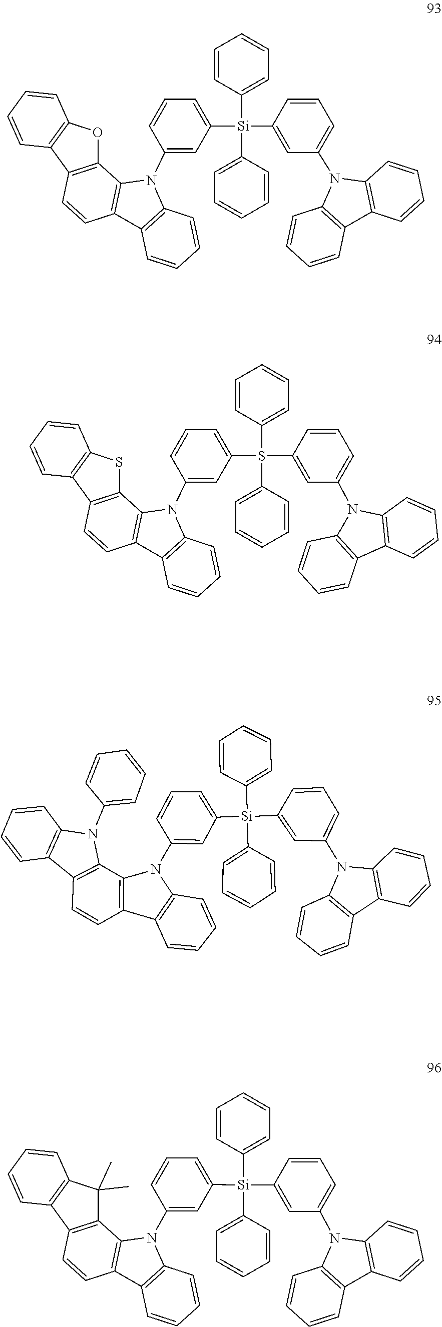 [SMILES: c1ccc([Si](c2ccccc2)(c2cccc(-n3c4ccccc4c4ccccc43)c2)c2cccc(-n3c4ccccc4c4ccc5c6ccccc6oc5c43)c2)cc1 ; c1ccc(S(c2ccccc2)(c2cccc(-n3c4ccccc4c4ccccc43)c2)c2cccc(-n3c4ccccc4c4ccc5c6ccccc6sc5c43)c2)cc1 ; c1ccc(-n2c3ccccc3c3ccc4c5ccccc5n(-c5cccc([Si](c6ccccc6)(c6ccccc6)c6cccc(-n7c8ccccc8c8ccccc87)c6)c5)c4c32)cc1 ; CC1(C)c2ccccc2-c2ccc3c4ccccc4n(-c4cccc([Si](c5ccccc5)(c5ccccc5)c5cccc(-n6c7ccccc7c7ccccc76)c5)c4)c3c21]

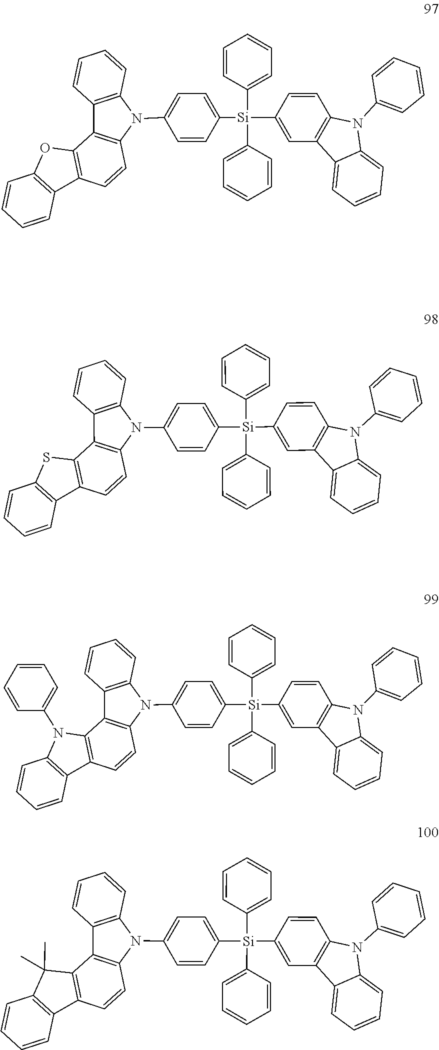 [SMILES: c1ccc(-n2c3ccccc3c3cc([Si](c4ccccc4)(c4ccccc4)c4ccc(-n5c6ccccc6c6c7oc8ccccc8c7ccc65)cc4)ccc32)cc1 ; c1ccc(-n2c3ccccc3c3cc([Si](c4ccccc4)(c4ccccc4)c4ccc(-n5c6ccccc6c6c7sc8ccccc8c7ccc65)cc4)ccc32)cc1 ; c1ccc(-n2c3ccccc3c3cc([Si](c4ccccc4)(c4ccccc4)c4ccc(-n5c6ccccc6c6c5ccc5c7ccccc7n(-c7ccccc7)c56)cc4)ccc32)cc1 ; CC1(C)c2ccccc2-c2ccc3c(c21)c1ccccc1n3-c1ccc([Si](c2ccccc2)(c2ccccc2)c2ccc3c(c2)c2ccccc2n3-c2ccccc2)cc1]

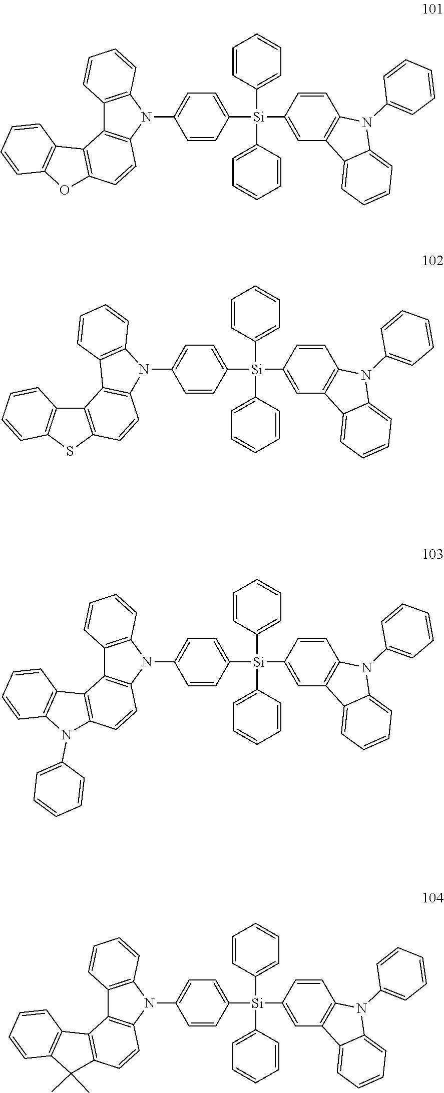 [SMILES: c1ccc(-n2c3ccccc3c3cc([Si](c4ccccc4)(c4ccccc4)c4ccc(-n5c6ccccc6c6c7c(ccc65)oc5ccccc57)cc4)ccc32)cc1 ; c1ccc(-n2c3ccccc3c3cc([Si](c4ccccc4)(c4ccccc4)c4ccc(-n5c6ccccc6c6c7c(ccc65)sc5ccccc57)cc4)ccc32)cc1 ; c1ccc(-n2c3ccccc3c3cc([Si](c4ccccc4)(c4ccccc4)c4ccc(-n5c6ccccc6c6c7c8ccccc8n(-c8ccccc8)c7ccc65)cc4)ccc32)cc1 ; CC1(C)c2ccccc2-c2c1ccc1c2c2ccccc2n1-c1ccc([Si](c2ccccc2)(c2ccccc2)c2ccc3c(c2)c2ccccc2n3-c2ccccc2)cc1]

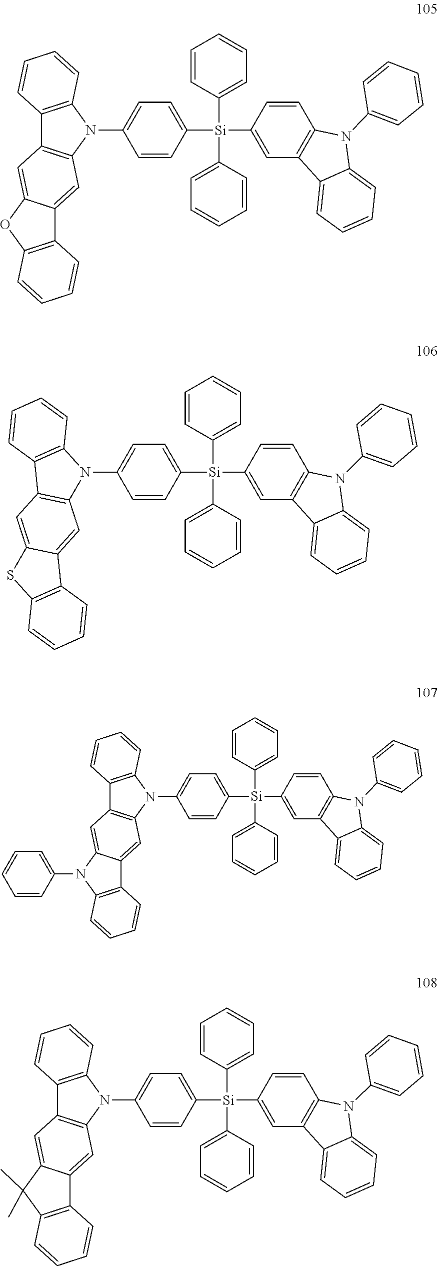 [SMILES: c1ccc(-n2c3ccccc3c3cc([Si](c4ccccc4)(c4ccccc4)c4ccc(-n5c6ccccc6c6cc7oc8ccccc8c7cc65)cc4)ccc32)cc1 ; c1ccc(-n2c3ccccc3c3cc([Si](c4ccccc4)(c4ccccc4)c4ccc(-n5c6ccccc6c6cc7sc8ccccc8c7cc65)cc4)ccc32)cc1 ; c1ccc(-n2c3ccccc3c3cc([Si](c4ccccc4)(c4ccccc4)c4ccc(-n5c6ccccc6c6cc7c(cc65)c5ccccc5n7-c5ccccc5)cc4)ccc32)cc1 ; CC1(C)c2ccccc2-c2cc3c(cc21)c1ccccc1n3-c1ccc([Si](c2ccccc2)(c2ccccc2)c2ccc3c(c2)c2ccccc2n3-c2ccccc2)cc1]

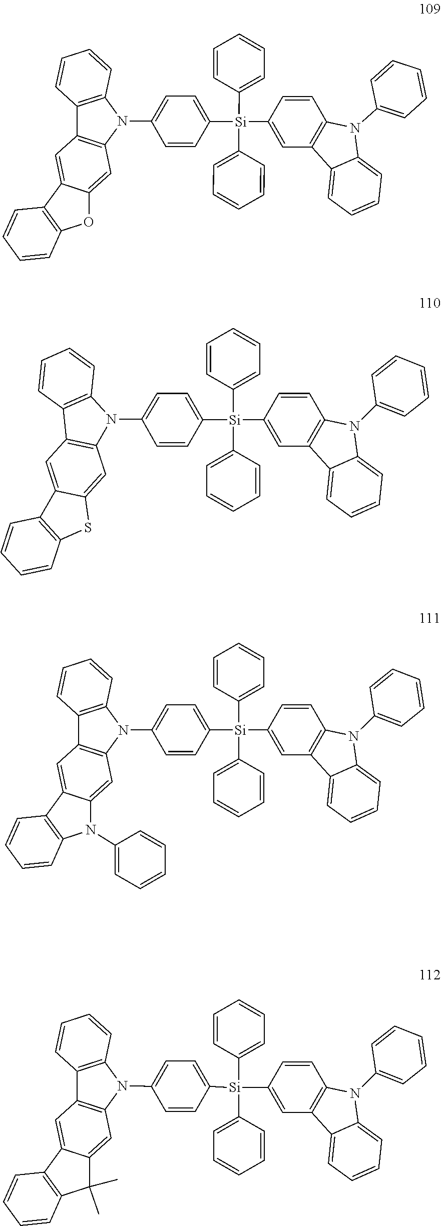 [SMILES: c1ccc(-n2c3ccccc3c3cc([Si](c4ccccc4)(c4ccccc4)c4ccc(-n5c6ccccc6c6cc7c(cc65)oc5ccccc57)cc4)ccc32)cc1 ; c1ccc(-n2c3ccccc3c3cc([Si](c4ccccc4)(c4ccccc4)c4ccc(-n5c6ccccc6c6cc7c(cc65)sc5ccccc57)cc4)ccc32)cc1 ; c1ccc(-n2c3ccccc3c3cc([Si](c4ccccc4)(c4ccccc4)c4ccc(-n5c6ccccc6c6cc7c8ccccc8n(-c8ccccc8)c7cc65)cc4)ccc32)cc1 ; CC1(C)c2ccccc2-c2cc3c4ccccc4n(-c4ccc([Si](c5ccccc5)(c5ccccc5)c5ccc6c(c5)c5ccccc5n6-c5ccccc5)cc4)c3cc21]

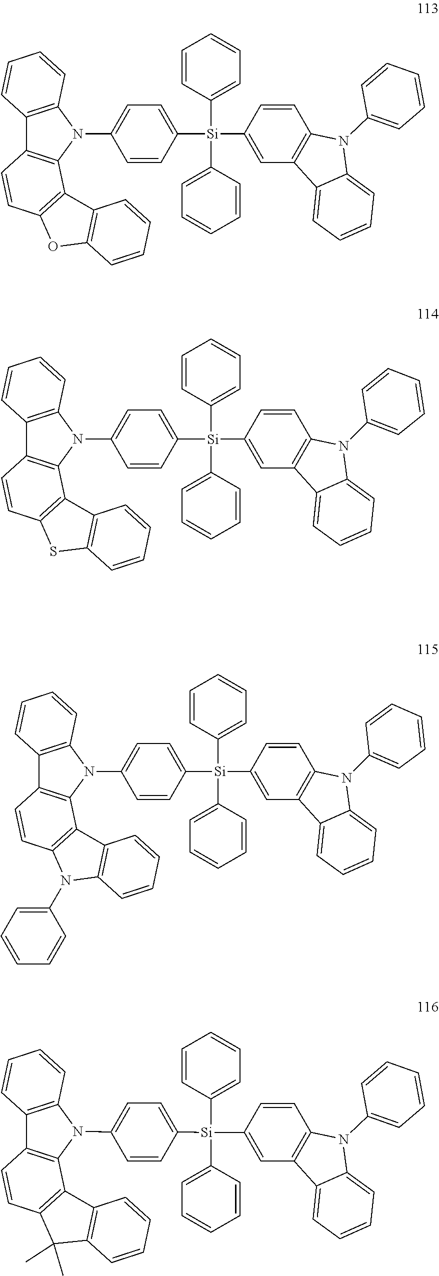 [SMILES: c1ccc(-n2c3ccccc3c3cc([Si](c4ccccc4)(c4ccccc4)c4ccc(-n5c6ccccc6c6ccc7oc8ccccc8c7c65)cc4)ccc32)cc1 ; c1ccc(-n2c3ccccc3c3cc([Si](c4ccccc4)(c4ccccc4)c4ccc(-n5c6ccccc6c6ccc7sc8ccccc8c7c65)cc4)ccc32)cc1 ; c1ccc(-n2c3ccccc3c3cc([Si](c4ccccc4)(c4ccccc4)c4ccc(-n5c6ccccc6c6ccc7c(c8ccccc8n7-c7ccccc7)c65)cc4)ccc32)cc1 ; CC1(C)c2ccccc2-c2c1ccc1c3ccccc3n(-c3ccc([Si](c4ccccc4)(c4ccccc4)c4ccc5c(c4)c4ccccc4n5-c4ccccc4)cc3)c21]

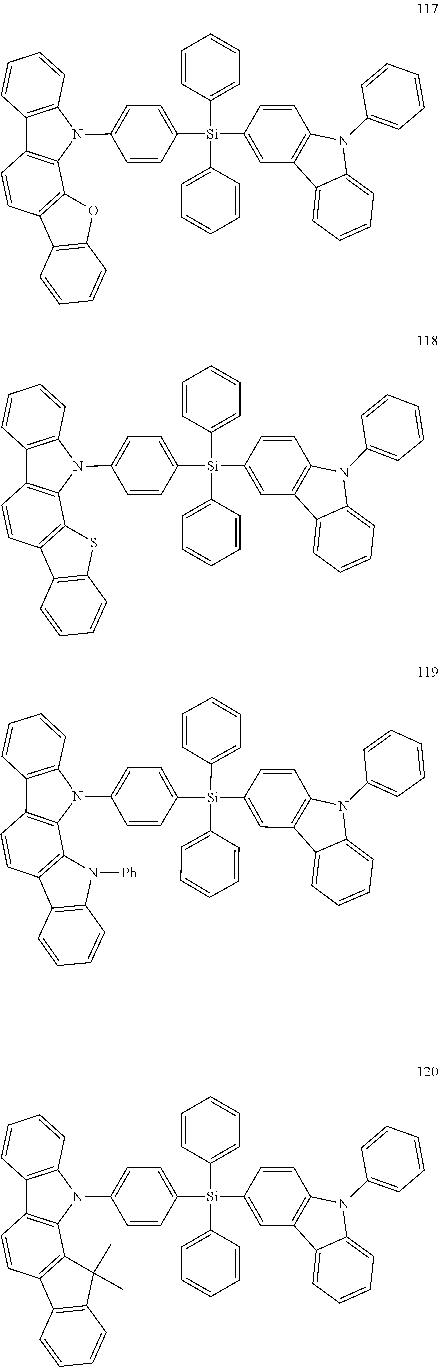 [SMILES: c1ccc(-n2c3ccccc3c3cc([Si](c4ccccc4)(c4ccccc4)c4ccc(-n5c6ccccc6c6ccc7c8ccccc8oc7c65)cc4)ccc32)cc1 ; c1ccc(-n2c3ccccc3c3cc([Si](c4ccccc4)(c4ccccc4)c4ccc(-n5c6ccccc6c6ccc7c8ccccc8sc7c65)cc4)ccc32)cc1 ; c1ccc(-n2c3ccccc3c3cc([Si](c4ccccc4)(c4ccccc4)c4ccc(-n5c6ccccc6c6ccc7c8ccccc8n(-c8ccccc8)c7c65)cc4)ccc32)cc1 ; CC1(C)c2ccccc2-c2ccc3c4ccccc4n(-c4ccc([Si](c5ccccc5)(c5ccccc5)c5ccc6c(c5)c5ccccc5n6-c5ccccc5)cc4)c3c21]

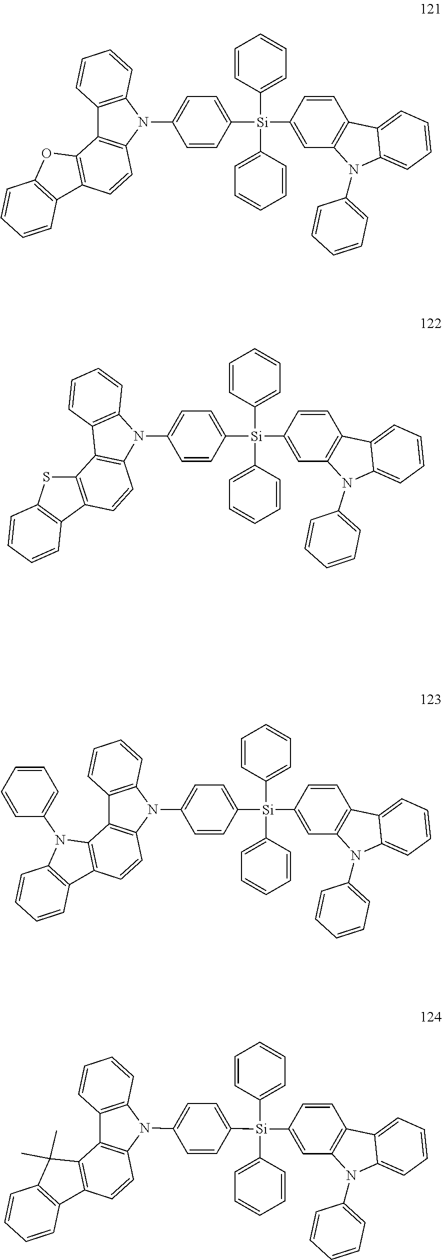 [SMILES: c1ccc(-n2c3ccccc3c3ccc([Si](c4ccccc4)(c4ccccc4)c4ccc(-n5c6ccccc6c6c7oc8ccccc8c7ccc65)cc4)cc32)cc1 ; c1ccc(-n2c3ccccc3c3ccc([Si](c4ccccc4)(c4ccccc4)c4ccc(-n5c6ccccc6c6c7sc8ccccc8c7ccc65)cc4)cc32)cc1 ; c1ccc(-n2c3ccccc3c3ccc([Si](c4ccccc4)(c4ccccc4)c4ccc(-n5c6ccccc6c6c5ccc5c7ccccc7n(-c7ccccc7)c56)cc4)cc32)cc1 ; CC1(C)c2ccccc2-c2ccc3c(c21)c1ccccc1n3-c1ccc([Si](c2ccccc2)(c2ccccc2)c2ccc3c4ccccc4n(-c4ccccc4)c3c2)cc1]

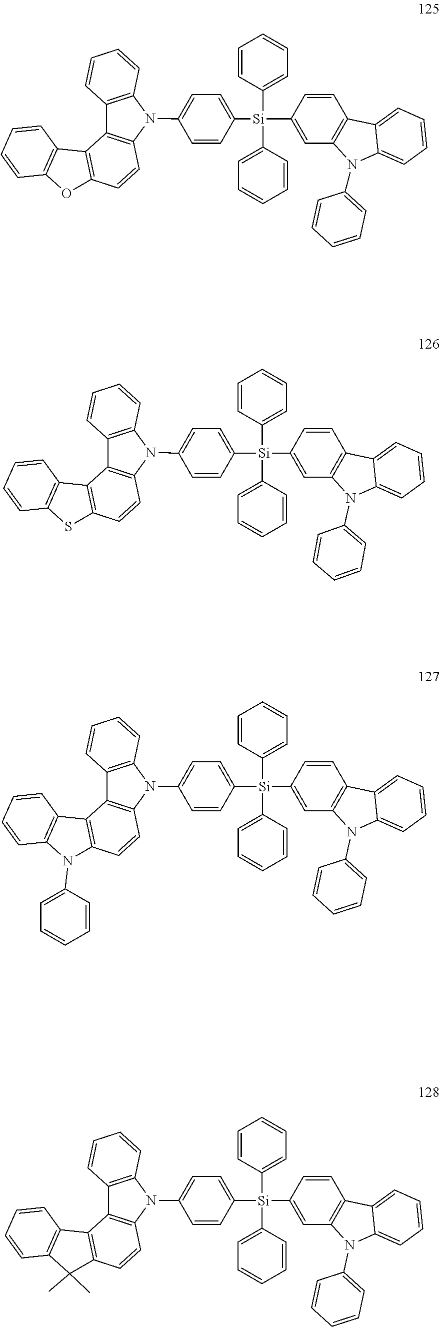 [SMILES: c1ccc(-n2c3ccccc3c3ccc([Si](c4ccccc4)(c4ccccc4)c4ccc(-n5c6ccccc6c6c7c(ccc65)oc5ccccc57)cc4)cc32)cc1 ; c1ccc(-n2c3ccccc3c3ccc([Si](c4ccccc4)(c4ccccc4)c4ccc(-n5c6ccccc6c6c7c(ccc65)sc5ccccc57)cc4)cc32)cc1 ; c1ccc(-n2c3ccccc3c3ccc([Si](c4ccccc4)(c4ccccc4)c4ccc(-n5c6ccccc6c6c7c8ccccc8n(-c8ccccc8)c7ccc65)cc4)cc32)cc1 ; CC1(C)c2ccccc2-c2c1ccc1c2c2ccccc2n1-c1ccc([Si](c2ccccc2)(c2ccccc2)c2ccc3c4ccccc4n(-c4ccccc4)c3c2)cc1]

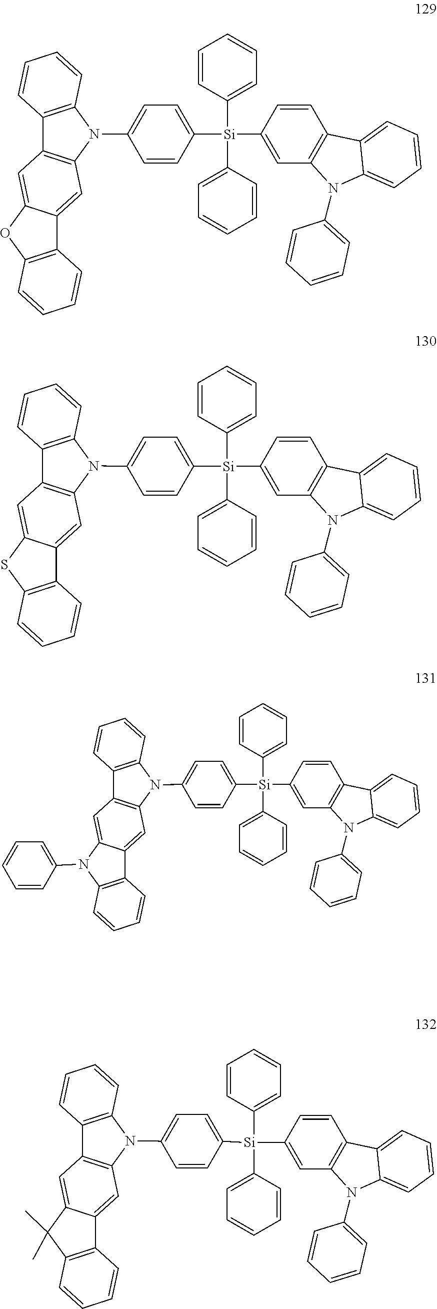 [SMILES: c1ccc(-n2c3ccccc3c3ccc([Si](c4ccccc4)(c4ccccc4)c4ccc(-n5c6ccccc6c6cc7oc8ccccc8c7cc65)cc4)cc32)cc1 ; c1ccc(-n2c3ccccc3c3ccc([Si](c4ccccc4)(c4ccccc4)c4ccc(-n5c6ccccc6c6cc7sc8ccccc8c7cc65)cc4)cc32)cc1 ; c1ccc(-n2c3ccccc3c3ccc([Si](c4ccccc4)(c4ccccc4)c4ccc(-n5c6ccccc6c6cc7c(cc65)c5ccccc5n7-c5ccccc5)cc4)cc32)cc1 ; CC1(C)c2ccccc2-c2cc3c(cc21)c1ccccc1n3-c1ccc([Si](c2ccccc2)(c2ccccc2)c2ccc3c4ccccc4n(-c4ccccc4)c3c2)cc1]

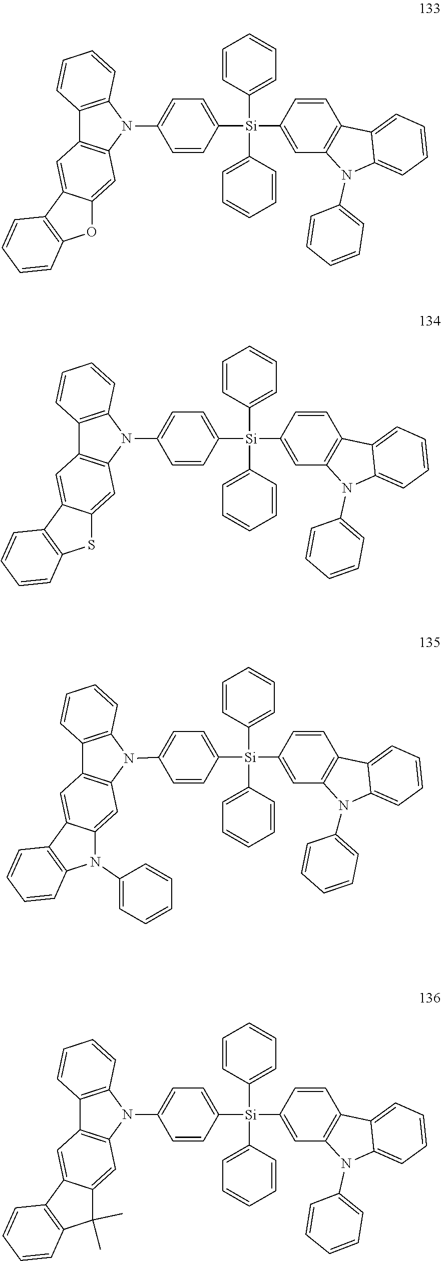 [SMILES: c1ccc(-n2c3ccccc3c3ccc([Si](c4ccccc4)(c4ccccc4)c4ccc(-n5c6ccccc6c6cc7c(cc65)oc5ccccc57)cc4)cc32)cc1 ; c1ccc(-n2c3ccccc3c3ccc([Si](c4ccccc4)(c4ccccc4)c4ccc(-n5c6ccccc6c6cc7c(cc65)sc5ccccc57)cc4)cc32)cc1 ; c1ccc(-n2c3ccccc3c3ccc([Si](c4ccccc4)(c4ccccc4)c4ccc(-n5c6ccccc6c6cc7c8ccccc8n(-c8ccccc8)c7cc65)cc4)cc32)cc1 ; CC1(C)c2ccccc2-c2cc3c4ccccc4n(-c4ccc([Si](c5ccccc5)(c5ccccc5)c5ccc6c7ccccc7n(-c7ccccc7)c6c5)cc4)c3cc21]

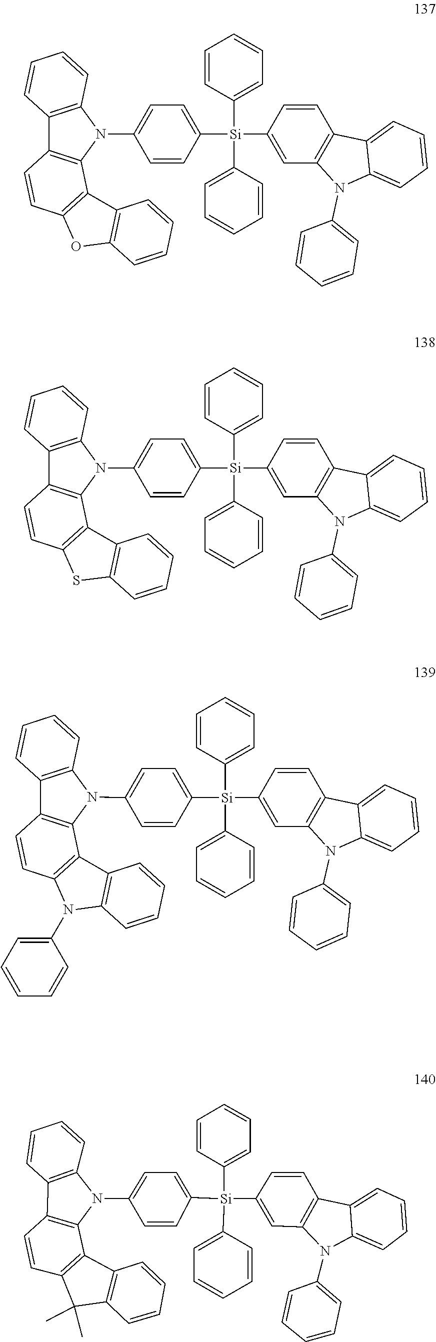 [SMILES: c1ccc(-n2c3ccccc3c3ccc([Si](c4ccccc4)(c4ccccc4)c4ccc(-n5c6ccccc6c6ccc7oc8ccccc8c7c65)cc4)cc32)cc1 ; c1ccc(-n2c3ccccc3c3ccc([Si](c4ccccc4)(c4ccccc4)c4ccc(-n5c6ccccc6c6ccc7sc8ccccc8c7c65)cc4)cc32)cc1 ; c1ccc(-n2c3ccccc3c3ccc([Si](c4ccccc4)(c4ccccc4)c4ccc(-n5c6ccccc6c6ccc7c(c8ccccc8n7-c7ccccc7)c65)cc4)cc32)cc1 ; CC1(C)c2ccccc2-c2c1ccc1c3ccccc3n(-c3ccc([Si](c4ccccc4)(c4ccccc4)c4ccc5c6ccccc6n(-c6ccccc6)c5c4)cc3)c21]

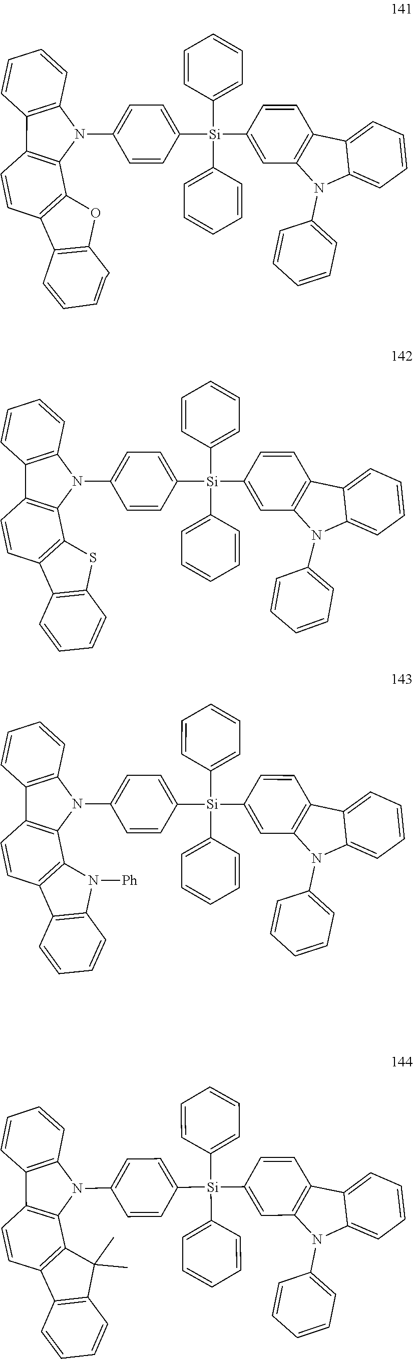 [SMILES: C1=CCc2oc3c(ccc4c5ccccc5n(-c5ccc([Si](c6ccccc6)(c6ccccc6)c6ccc7c8ccccc8n(-c8ccccc8)c7c6)cc5)c43)c2C=C1 ; c1ccc(-n2c3ccccc3c3ccc([Si](c4ccccc4)(c4ccccc4)c4ccc(-n5c6ccccc6c6ccc7c8ccccc8sc7c65)cc4)cc32)cc1 ; c1ccc(-n2c3ccccc3c3ccc([Si](c4ccccc4)(c4ccccc4)c4ccc(-n5c6ccccc6c6ccc7c8ccccc8n(-c8ccccc8)c7c65)cc4)cc32)cc1 ; CC1(C)c2ccccc2-c2ccc3c4ccccc4n(-c4ccc([Si](c5ccccc5)(c5ccccc5)c5ccc6c7ccccc7n(-c7ccccc7)c6c5)cc4)c3c21]

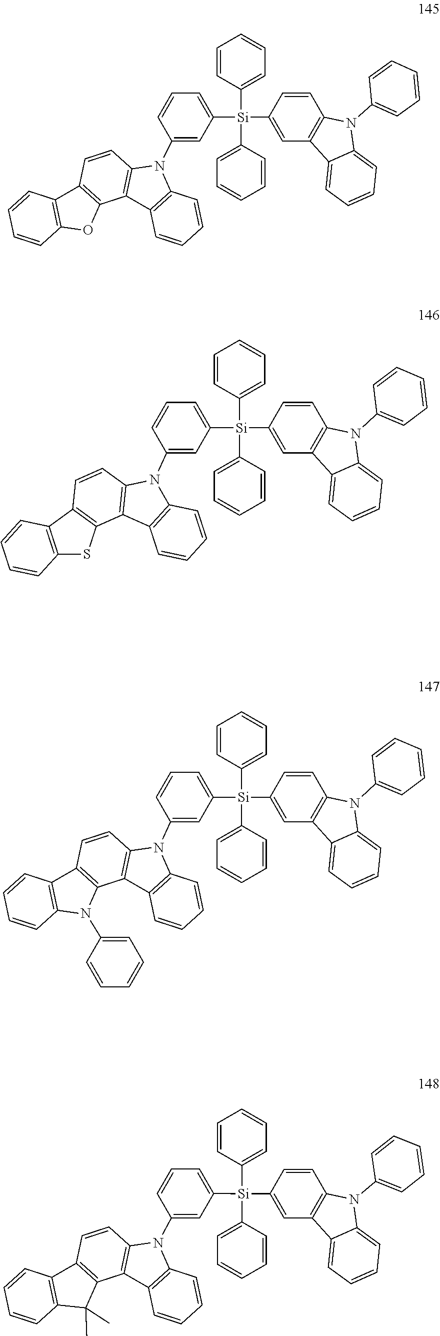 [SMILES: c1ccc(-n2c3ccccc3c3cc([Si](c4ccccc4)(c4ccccc4)c4cccc(-n5c6ccccc6c6c7oc8ccccc8c7ccc65)c4)ccc32)cc1 ; c1ccc(-n2c3ccccc3c3cc([Si](c4ccccc4)(c4ccccc4)c4cccc(-n5c6ccccc6c6c7sc8ccccc8c7ccc65)c4)ccc32)cc1 ; c1ccc(-n2c3ccccc3c3cc([Si](c4ccccc4)(c4ccccc4)c4cccc(-n5c6ccccc6c6c5ccc5c7ccccc7n(-c7ccccc7)c56)c4)ccc32)cc1 ; CC1(C)c2ccccc2-c2ccc3c(c21)c1ccccc1n3-c1cccc([Si](c2ccccc2)(c2ccccc2)c2ccc3c(c2)c2ccccc2n3-c2ccccc2)c1]

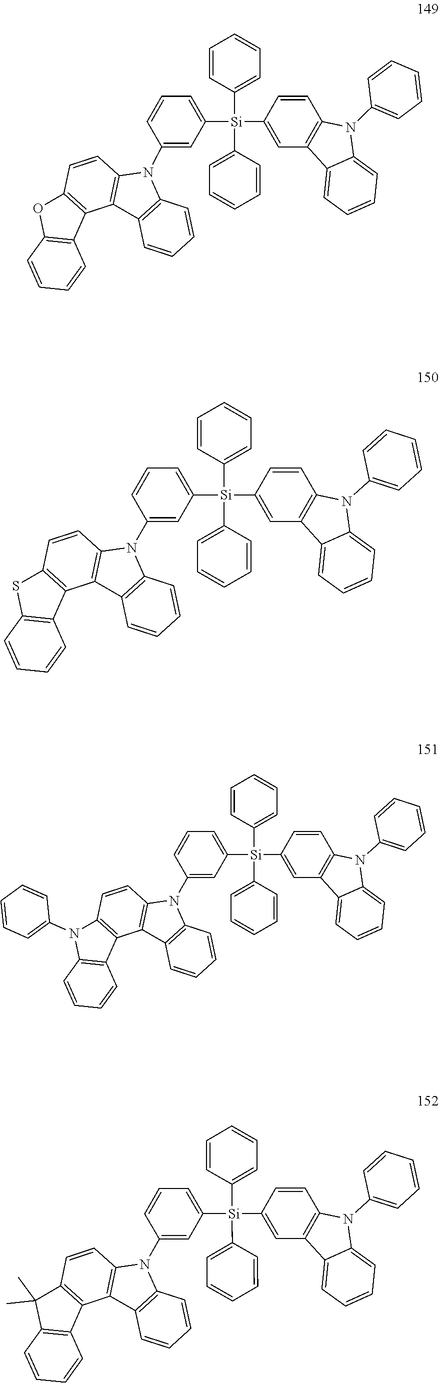 [SMILES: c1ccc(-n2c3ccccc3c3cc([Si](c4ccccc4)(c4ccccc4)c4cccc(-n5c6ccccc6c6c7c(ccc65)oc5ccccc57)c4)ccc32)cc1 ; c1ccc(-n2c3ccccc3c3cc([Si](c4ccccc4)(c4ccccc4)c4cccc(-n5c6ccccc6c6c7c(ccc65)sc5ccccc57)c4)ccc32)cc1 ; c1ccc(-n2c3ccccc3c3cc([Si](c4ccccc4)(c4ccccc4)c4cccc(-n5c6ccccc6c6c7c8ccccc8n(-c8ccccc8)c7ccc65)c4)ccc32)cc1 ; CC1(C)c2ccccc2-c2c1ccc1c2c2ccccc2n1-c1cccc([Si](c2ccccc2)(c2ccccc2)c2ccc3c(c2)c2ccccc2n3-c2ccccc2)c1]

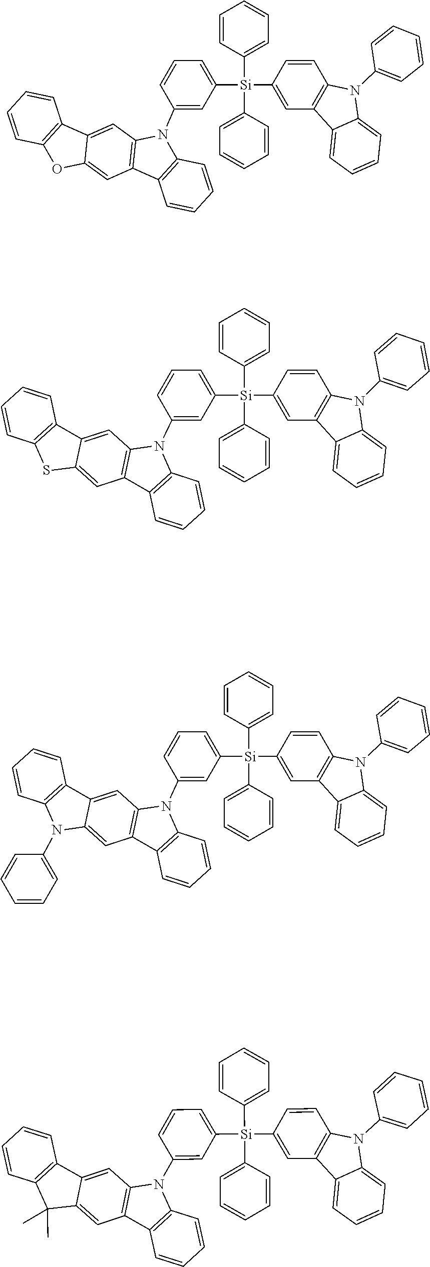 [SMILES: c1ccc(-n2c3ccccc3c3cc([Si](c4ccccc4)(c4ccccc4)c4cccc(-n5c6ccccc6c6cc7oc8ccccc8c7cc65)c4)ccc32)cc1 ; c1ccc(-n2c3ccccc3c3cc([Si](c4ccccc4)(c4ccccc4)c4cccc(-n5c6ccccc6c6cc7sc8ccccc8c7cc65)c4)ccc32)cc1 ; c1ccc(-n2c3ccccc3c3cc([Si](c4ccccc4)(c4ccccc4)c4cccc(-n5c6ccccc6c6cc7c(cc65)c5ccccc5n7-c5ccccc5)c4)ccc32)cc1 ; CC1(C)c2ccccc2-c2cc3c(cc21)c1ccccc1n3-c1cccc([Si](c2ccccc2)(c2ccccc2)c2ccc3c(c2)c2ccccc2n3-c2ccccc2)c1]

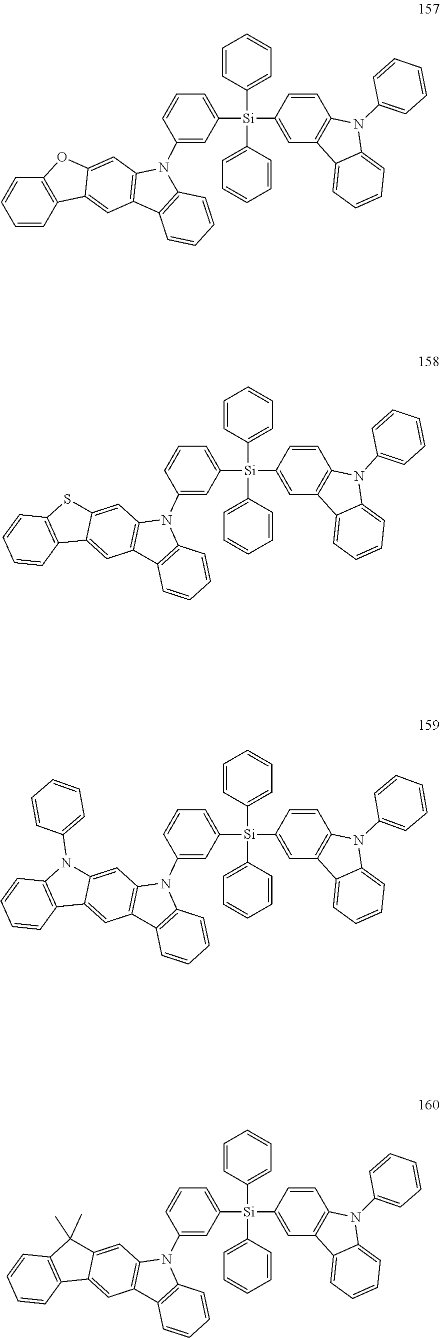 [SMILES: c1ccc(-n2c3ccccc3c3cc([Si](c4ccccc4)(c4ccccc4)c4cccc(-n5c6ccccc6c6cc7c(cc65)oc5ccccc57)c4)ccc32)cc1 ; c1ccc(-n2c3ccccc3c3cc([Si](c4ccccc4)(c4ccccc4)c4cccc(-n5c6ccccc6c6cc7c(cc65)sc5ccccc57)c4)ccc32)cc1 ; c1ccc(-n2c3ccccc3c3cc([Si](c4ccccc4)(c4ccccc4)c4cccc(-n5c6ccccc6c6cc7c8ccccc8n(-c8ccccc8)c7cc65)c4)ccc32)cc1 ; CC1(C)c2ccccc2-c2cc3c4ccccc4n(-c4cccc([Si](c5ccccc5)(c5ccccc5)c5ccc6c(c5)c5ccccc5n6-c5ccccc5)c4)c3cc21]

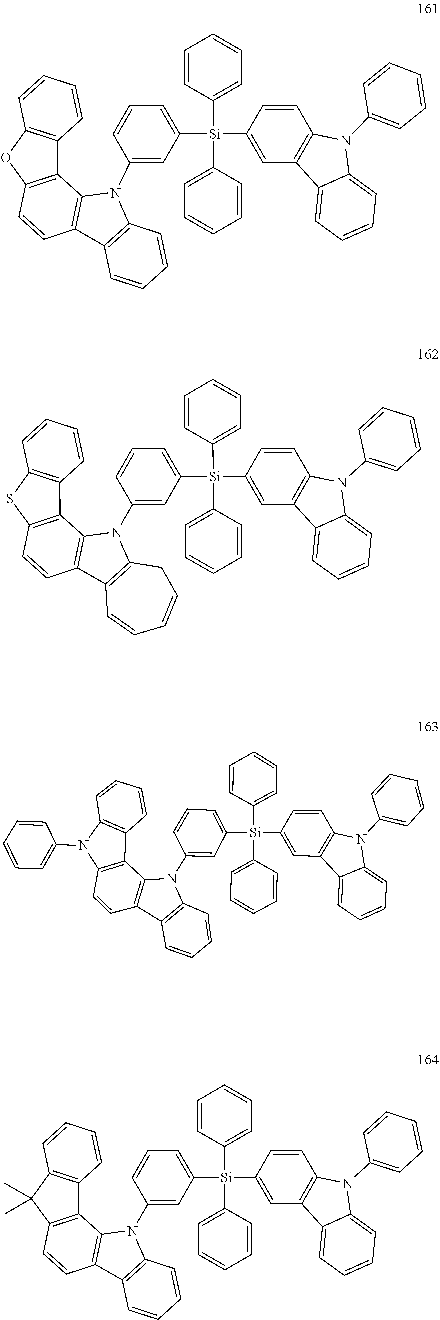 [SMILES: c1ccc(-n2c3ccccc3c3cc([Si](c4ccccc4)(c4ccccc4)c4cccc(-n5c6ccccc6c6ccc7oc8ccccc8c7c65)c4)ccc32)cc1 ; C1=CCc2c(c3ccc4sc5ccccc5c4c3n2-c2cccc([Si](c3ccccc3)(c3ccccc3)c3ccc4c(c3)c3ccccc3n4-c3ccccc3)c2)C=C1 ; C1=CCC=CC(n2c3ccccc3c3cc([Si](c4ccccc4)(c4ccccc4)c4cccc(-n5c6c(c7ccc8c(c9ccccc9n8-c8ccccc8)c75)C=CC=CC6)c4)ccc32)=C1 ; CC1(C)c2ccccc2-c2c1ccc1c3ccccc3n(-c3cccc([Si](c4ccccc4)(c4ccccc4)c4ccc5c(c4)c4ccccc4n5-c4ccccc4)c3)c21]

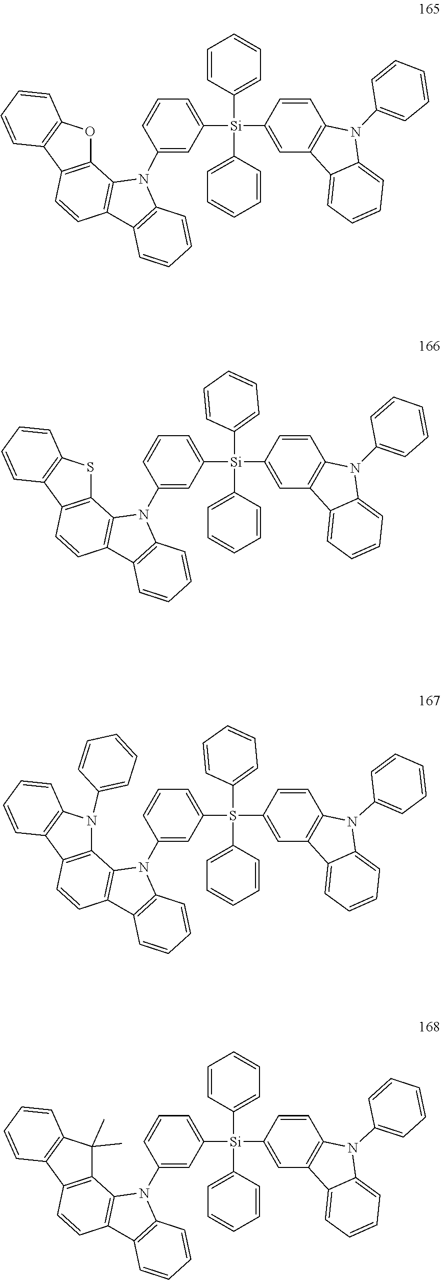 [SMILES: c1ccc(-n2c3ccccc3c3cc([Si](c4ccccc4)(c4ccccc4)c4cccc(-n5c6ccccc6c6ccc7c8ccccc8oc7c65)c4)ccc32)cc1 ; c1ccc(-n2c3ccccc3c3cc([Si](c4ccccc4)(c4ccccc4)c4cccc(-n5c6ccccc6c6ccc7c8ccccc8sc7c65)c4)ccc32)cc1 ; c1ccc(-n2c3ccccc3c3cc(S(c4ccccc4)(c4ccccc4)c4cccc(-n5c6ccccc6c6ccc7c8ccccc8n(-c8ccccc8)c7c65)c4)ccc32)cc1 ; CC1(C)c2ccccc2-c2ccc3c4ccccc4n(-c4cccc([Si](c5ccccc5)(c5ccccc5)c5ccc6c(c5)c5ccccc5n6-c5ccccc5)c4)c3c21]

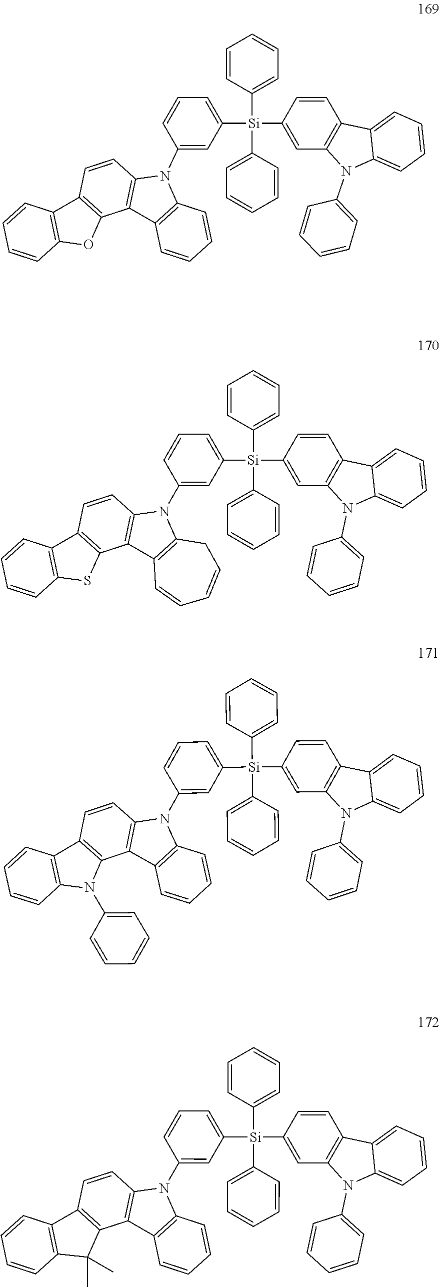 [SMILES: c1ccc(-n2c3ccccc3c3ccc([Si](c4ccccc4)(c4ccccc4)c4cccc(-n5c6ccccc6c6c7oc8ccccc8c7ccc65)c4)cc32)cc1 ; C1=CCc2c(c3c4sc5ccccc5c4ccc3n2-c2cccc([Si](c3ccccc3)(c3ccccc3)c3ccc4c5ccccc5n(-c5ccccc5)c4c3)c2)C=C1 ; c1ccc(-n2c3ccccc3c3ccc([Si](c4ccccc4)(c4ccccc4)c4cccc(-n5c6ccccc6c6c5ccc5c7ccccc7n(-c7ccccc7)c56)c4)cc32)cc1 ; CC1(C)c2ccccc2-c2ccc3c(c21)c1ccccc1n3-c1cccc([Si](c2ccccc2)(c2ccccc2)c2ccc3c4ccccc4n(-c4ccccc4)c3c2)c1]

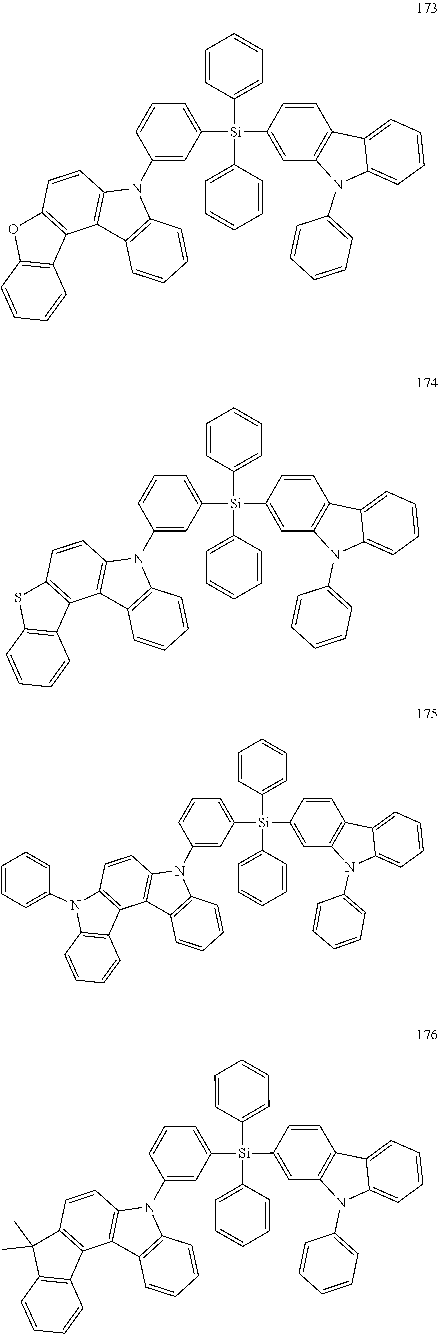 [SMILES: c1ccc(-n2c3ccccc3c3ccc([Si](c4ccccc4)(c4ccccc4)c4cccc(-n5c6ccccc6c6c7c(ccc65)oc5ccccc57)c4)cc32)cc1 ; c1ccc(-n2c3ccccc3c3ccc([Si](c4ccccc4)(c4ccccc4)c4cccc(-n5c6ccccc6c6c7c(ccc65)sc5ccccc57)c4)cc32)cc1 ; c1ccc(-n2c3ccccc3c3ccc([Si](c4ccccc4)(c4ccccc4)c4cccc(-n5c6ccccc6c6c7c8ccccc8n(-c8ccccc8)c7ccc65)c4)cc32)cc1 ; CC1(C)c2ccccc2-c2c1ccc1c2c2ccccc2n1-c1cccc([Si](c2ccccc2)(c2ccccc2)c2ccc3c4ccccc4n(-c4ccccc4)c3c2)c1]

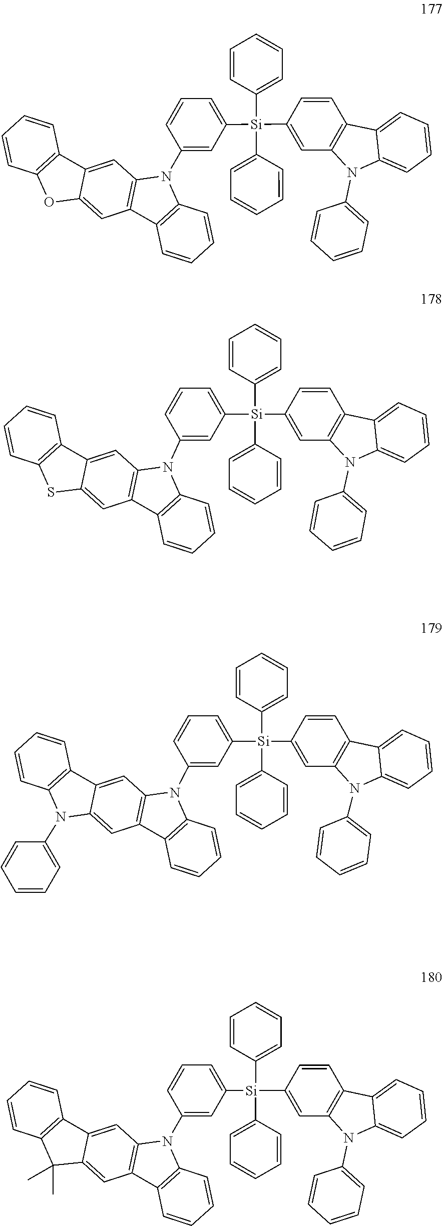 [SMILES: c1ccc(-n2c3ccccc3c3ccc([Si](c4ccccc4)(c4ccccc4)c4cccc(-n5c6ccccc6c6cc7oc8ccccc8c7cc65)c4)cc32)cc1 ; c1ccc(-n2c3ccccc3c3ccc([Si](c4ccccc4)(c4ccccc4)c4cccc(-n5c6ccccc6c6cc7sc8ccccc8c7cc65)c4)cc32)cc1 ; c1ccc(-n2c3ccccc3c3ccc([Si](c4ccccc4)(c4ccccc4)c4cccc(-n5c6ccccc6c6cc7c(cc65)c5ccccc5n7-c5ccccc5)c4)cc32)cc1 ; CC1(C)c2ccccc2-c2cc3c(cc21)c1ccccc1n3-c1cccc([Si](c2ccccc2)(c2ccccc2)c2ccc3c4ccccc4n(-c4ccccc4)c3c2)c1]

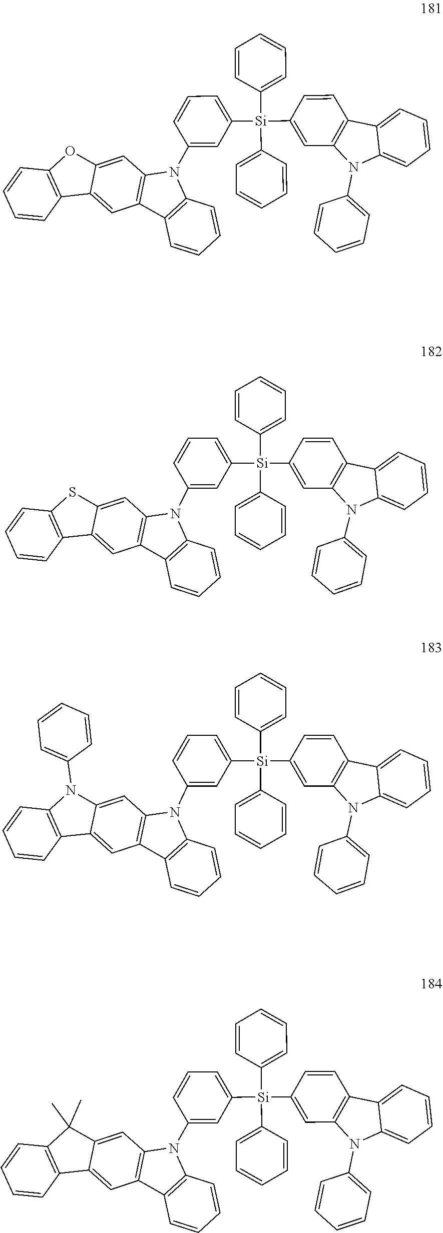 [SMILES: c1ccc(-n2c3ccccc3c3ccc([Si](c4ccccc4)(c4ccccc4)c4cccc(-n5c6ccccc6c6cc7c(cc65)oc5ccccc57)c4)cc32)cc1 ; c1ccc(-n2c3ccccc3c3ccc([Si](c4ccccc4)(c4ccccc4)c4cccc(-n5c6ccccc6c6cc7c(cc65)sc5ccccc57)c4)cc32)cc1 ; c1ccc(-n2c3ccccc3c3ccc([Si](c4ccccc4)(c4ccccc4)c4cccc(-n5c6ccccc6c6cc7c8ccccc8n(-c8ccccc8)c7cc65)c4)cc32)cc1 ; CC1(C)c2ccccc2-c2cc3c4ccccc4n(-c4cccc([Si](c5ccccc5)(c5ccccc5)c5ccc6c7ccccc7n(-c7ccccc7)c6c5)c4)c3cc21]

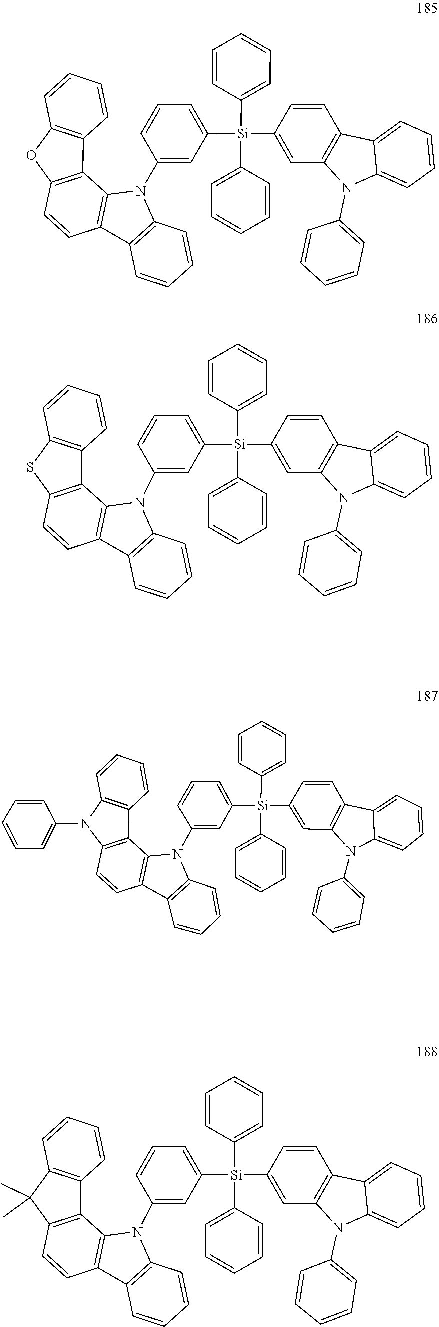 [SMILES: c1ccc(-n2c3ccccc3c3ccc([Si](c4ccccc4)(c4ccccc4)c4cccc(-n5c6ccccc6c6ccc7oc8ccccc8c7c65)c4)cc32)cc1 ; c1ccc(-n2c3ccccc3c3ccc([Si](c4ccccc4)(c4ccccc4)c4cccc(-n5c6ccccc6c6ccc7sc8ccccc8c7c65)c4)cc32)cc1 ; c1ccc(-n2c3ccccc3c3ccc([Si](c4ccccc4)(c4ccccc4)c4cccc(-n5c6ccccc6c6ccc7c(c8ccccc8n7-c7ccccc7)c65)c4)cc32)cc1 ; CC1(C)c2ccccc2-c2c1ccc1c3ccccc3n(-c3cccc([Si](c4ccccc4)(c4ccccc4)c4ccc5c6ccccc6n(-c6ccccc6)c5c4)c3)c21]

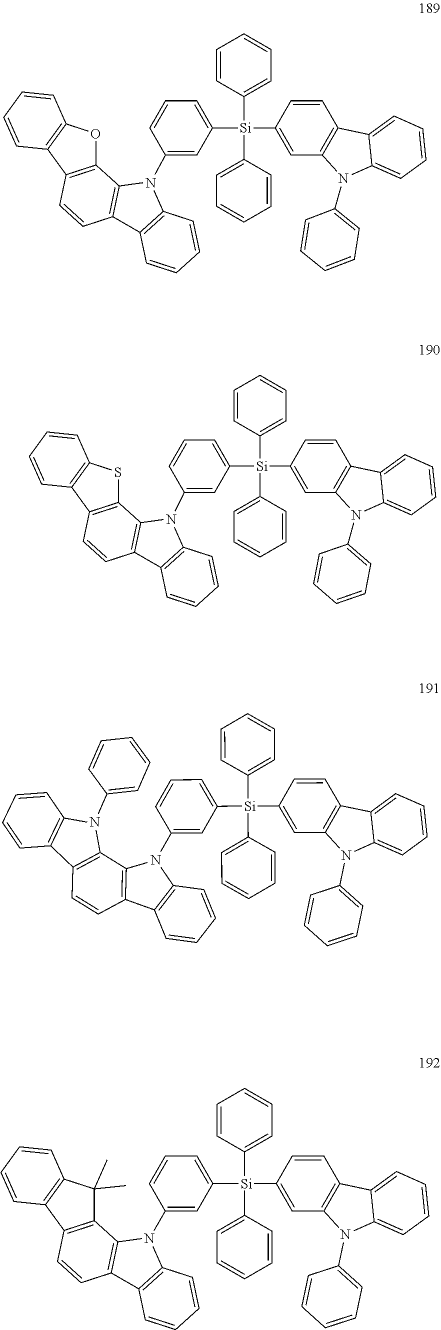 [SMILES: c1ccc(-n2c3ccccc3c3ccc([Si](c4ccccc4)(c4ccccc4)c4cccc(-n5c6ccccc6c6ccc7c8ccccc8oc7c65)c4)cc32)cc1 ; c1ccc(-n2c3ccccc3c3ccc([Si](c4ccccc4)(c4ccccc4)c4cccc(-n5c6ccccc6c6ccc7c8ccccc8sc7c65)c4)cc32)cc1 ; c1ccc(-n2c3ccccc3c3ccc([Si](c4ccccc4)(c4ccccc4)c4cccc(-n5c6ccccc6c6ccc7c8ccccc8n(-c8ccccc8)c7c65)c4)cc32)cc1 ; CC1(C)c2ccccc2-c2ccc3c4ccccc4n(-c4cccc([Si](c5ccccc5)(c5ccccc5)c5ccc6c7ccccc7n(-c7ccccc7)c6c5)c4)c3c21]

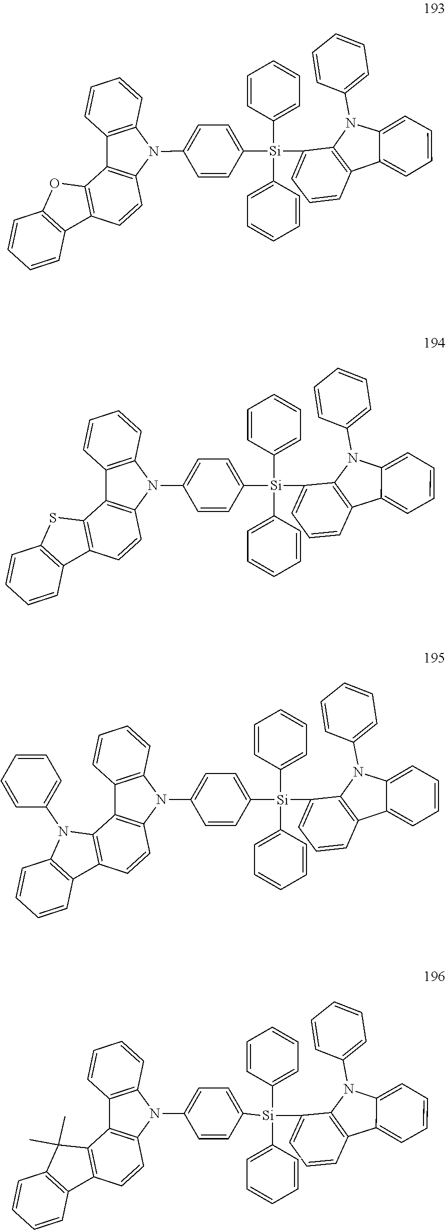 [SMILES: c1ccc(-n2c3ccccc3c3cccc([Si](c4ccccc4)(c4ccccc4)c4ccc(-n5c6ccccc6c6c7oc8ccccc8c7ccc65)cc4)c32)cc1 ; c1ccc(-n2c3ccccc3c3cccc([Si](c4ccccc4)(c4ccccc4)c4ccc(-n5c6ccccc6c6c7sc8ccccc8c7ccc65)cc4)c32)cc1 ; c1ccc(-n2c3ccccc3c3cccc([Si](c4ccccc4)(c4ccccc4)c4ccc(-n5c6ccccc6c6c5ccc5c7ccccc7n(-c7ccccc7)c56)cc4)c32)cc1 ; CC1(C)c2ccccc2-c2ccc3c(c21)c1ccccc1n3-c1ccc([Si](c2ccccc2)(c2ccccc2)c2cccc3c4ccccc4n(-c4ccccc4)c23)cc1]

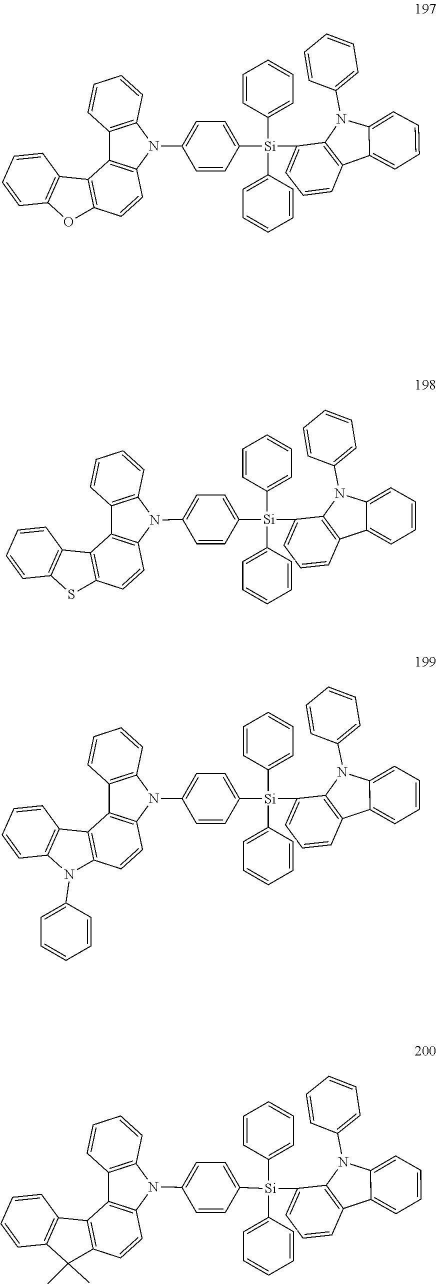 [SMILES: c1ccc(-n2c3ccccc3c3cccc([Si](c4ccccc4)(c4ccccc4)c4ccc(-n5c6ccccc6c6c7c(ccc65)oc5ccccc57)cc4)c32)cc1 ; c1ccc(-n2c3ccccc3c3cccc([Si](c4ccccc4)(c4ccccc4)c4ccc(-n5c6ccccc6c6c7c(ccc65)sc5ccccc57)cc4)c32)cc1 ; c1ccc(-n2c3ccccc3c3c4c5ccccc5n(-c5ccc([Si](c6ccccc6)(c6ccccc6)c6cccc7c8ccccc8n(-c8ccccc8)c67)cc5)c4ccc32)cc1 ; CC1(C)c2ccccc2-c2c1ccc1c2c2ccccc2n1-c1ccc([Si](c2ccccc2)(c2ccccc2)c2cccc3c4ccccc4n(-c4ccccc4)c23)cc1]

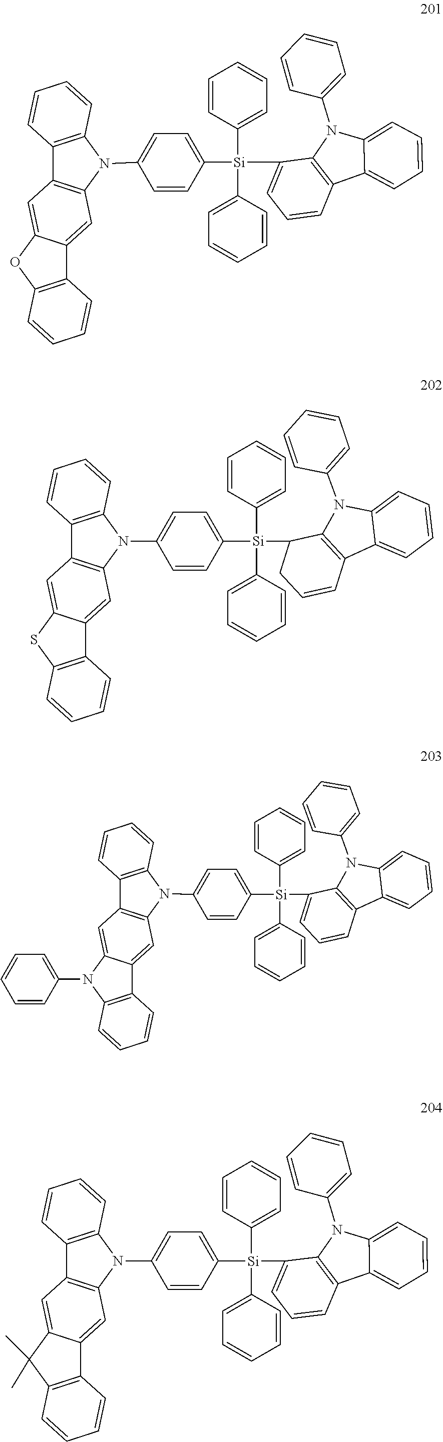 [SMILES: c1ccc(-n2c3ccccc3c3cccc([Si](c4ccccc4)(c4ccccc4)c4ccc(-n5c6ccccc6c6cc7oc8ccccc8c7cc65)cc4)c32)cc1 ; C1=Cc2c(n(-c3ccccc3)c3ccccc23)C([Si](c2ccccc2)(c2ccccc2)c2ccc(-n3c4ccccc4c4cc5sc6ccccc6c5cc43)cc2)C1 ; c1ccc(-n2c3ccccc3c3cc4c(cc32)c2ccccc2n4-c2ccc([Si](c3ccccc3)(c3ccccc3)c3cccc4c5ccccc5n(-c5ccccc5)c34)cc2)cc1 ; CC1(C)c2ccccc2-c2cc3c(cc21)c1ccccc1n3-c1ccc([Si](c2ccccc2)(c2ccccc2)c2cccc3c4ccccc4n(-c4ccccc4)c23)cc1]

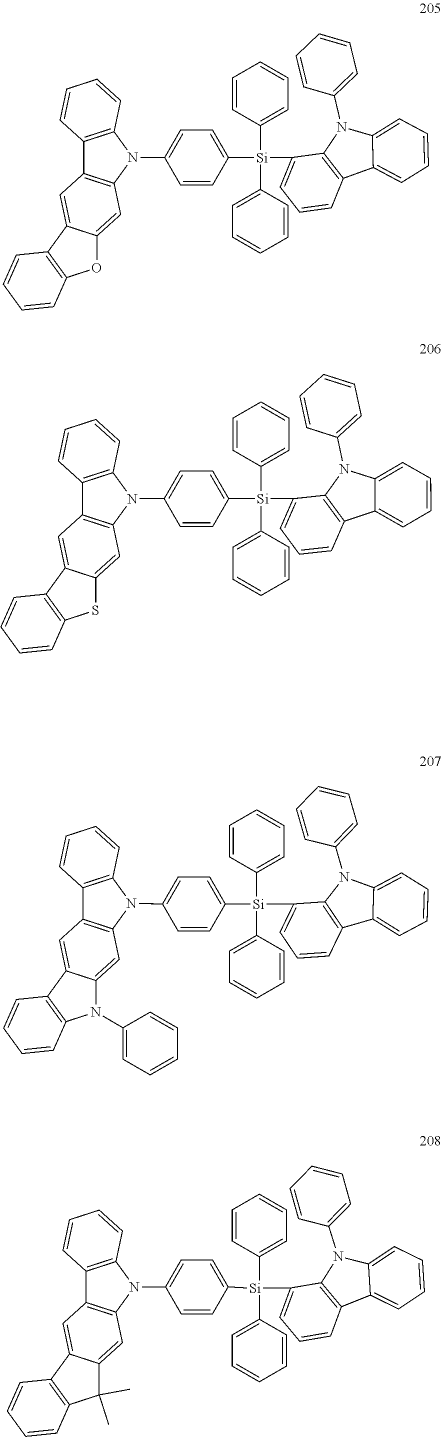 [SMILES: c1ccc(-n2c3ccccc3c3cccc([Si](c4ccccc4)(c4ccccc4)c4ccc(-n5c6ccccc6c6cc7c(cc65)oc5ccccc57)cc4)c32)cc1 ; c1ccc(-n2c3ccccc3c3cccc([Si](c4ccccc4)(c4ccccc4)c4ccc(-n5c6ccccc6c6cc7c(cc65)sc5ccccc57)cc4)c32)cc1 ; c1ccc(-n2c3ccccc3c3cc4c5ccccc5n(-c5ccc([Si](c6ccccc6)(c6ccccc6)c6cccc7c8ccccc8n(-c8ccccc8)c67)cc5)c4cc32)cc1 ; CC1(C)c2ccccc2-c2cc3c4ccccc4n(-c4ccc([Si](c5ccccc5)(c5ccccc5)c5cccc6c7ccccc7n(-c7ccccc7)c56)cc4)c3cc21]

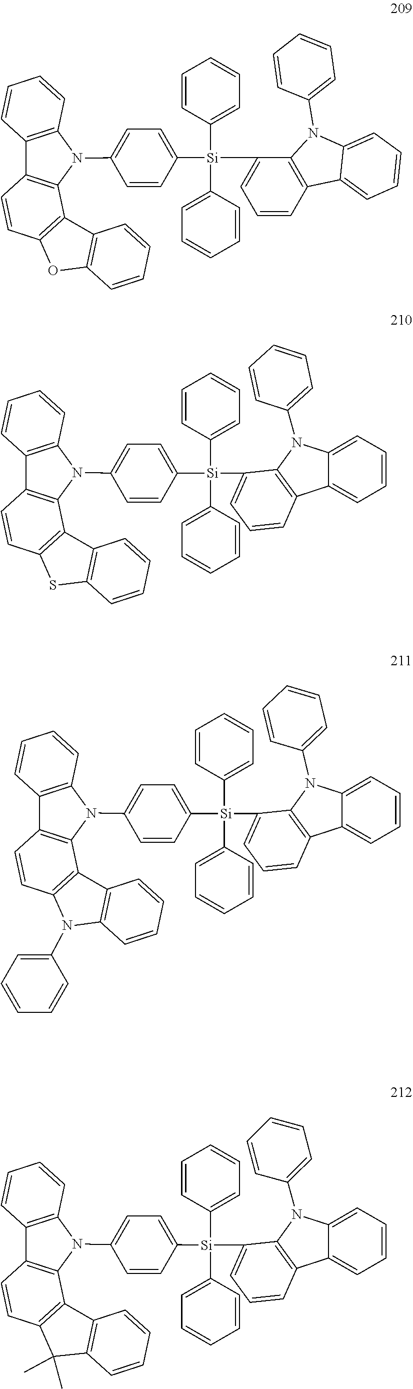 [SMILES: c1ccc(-n2c3ccccc3c3cccc([Si](c4ccccc4)(c4ccccc4)c4ccc(-n5c6ccccc6c6ccc7oc8ccccc8c7c65)cc4)c32)cc1 ; c1ccc(-n2c3ccccc3c3cccc([Si](c4ccccc4)(c4ccccc4)c4ccc(-n5c6ccccc6c6ccc7sc8ccccc8c7c65)cc4)c32)cc1 ; c1ccc(-n2c3ccccc3c3c2ccc2c4ccccc4n(-c4ccc([Si](c5ccccc5)(c5ccccc5)c5cccc6c7ccccc7n(-c7ccccc7)c56)cc4)c23)cc1 ; CC1(C)c2ccccc2-c2c1ccc1c3ccccc3n(-c3ccc([Si](c4ccccc4)(c4ccccc4)c4cccc5c6ccccc6n(-c6ccccc6)c45)cc3)c21]

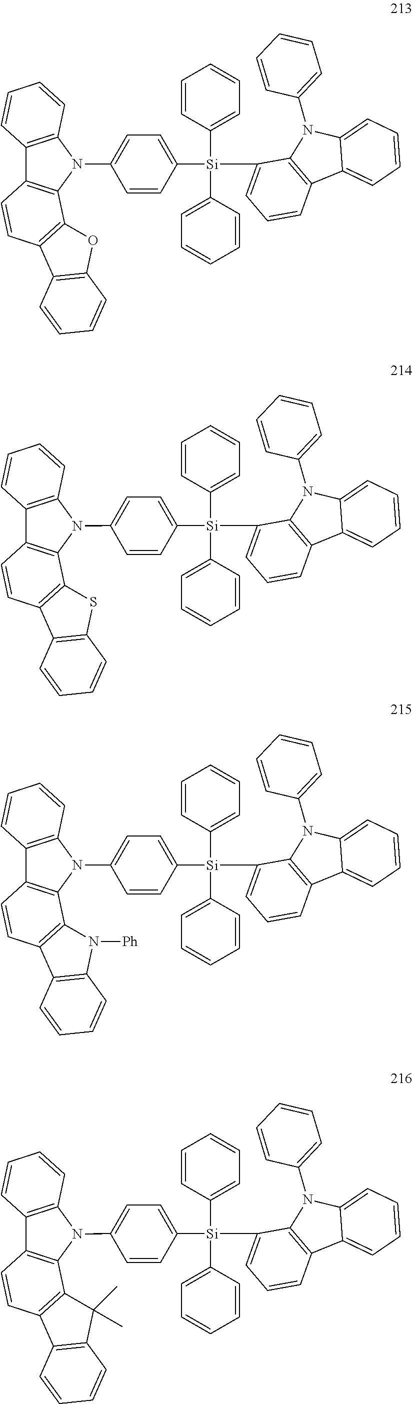 [SMILES: c1ccc(-n2c3ccccc3c3cccc([Si](c4ccccc4)(c4ccccc4)c4ccc(-n5c6ccccc6c6ccc7c8ccccc8oc7c65)cc4)c32)cc1 ; c1ccc(-n2c3ccccc3c3cccc([Si](c4ccccc4)(c4ccccc4)c4ccc(-n5c6ccccc6c6ccc7c8ccccc8sc7c65)cc4)c32)cc1 ; c1ccc(-n2c3ccccc3c3cccc([Si](c4ccccc4)(c4ccccc4)c4ccc(-n5c6ccccc6c6ccc7c8ccccc8n(-c8ccccc8)c7c65)cc4)c32)cc1 ; CC1(C)c2ccccc2-c2ccc3c4ccccc4n(-c4ccc([Si](c5ccccc5)(c5ccccc5)c5cccc6c7ccccc7n(-c7ccccc7)c56)cc4)c3c21]

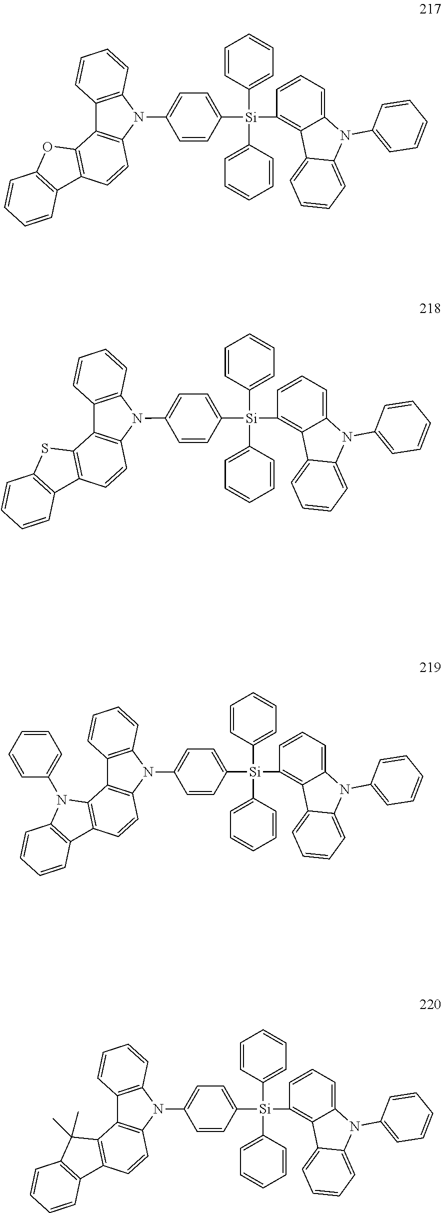 [SMILES: c1ccc(-n2c3ccccc3c3c([Si](c4ccccc4)(c4ccccc4)c4ccc(-n5c6ccccc6c6c7oc8ccccc8c7ccc65)cc4)cccc32)cc1 ; c1ccc(-n2c3ccccc3c3c([Si](c4ccccc4)(c4ccccc4)c4ccc(-n5c6ccccc6c6c7sc8ccccc8c7ccc65)cc4)cccc32)cc1 ; c1ccc(-n2c3ccccc3c3c([Si](c4ccccc4)(c4ccccc4)c4ccc(-n5c6ccccc6c6c5ccc5c7ccccc7n(-c7ccccc7)c56)cc4)cccc32)cc1 ; CC1(C)c2ccccc2-c2ccc3c(c21)c1ccccc1n3-c1ccc([Si](c2ccccc2)(c2ccccc2)c2cccc3c2c2ccccc2n3-c2ccccc2)cc1]

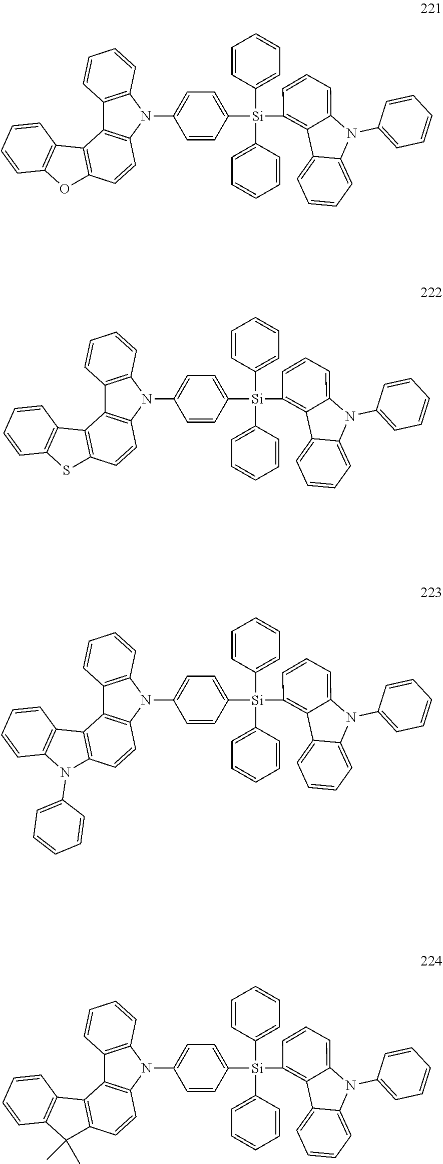 [SMILES: c1ccc(-n2c3ccccc3c3c([Si](c4ccccc4)(c4ccccc4)c4ccc(-n5c6ccccc6c6c7c(ccc65)oc5ccccc57)cc4)cccc32)cc1 ; c1ccc(-n2c3ccccc3c3c([Si](c4ccccc4)(c4ccccc4)c4ccc(-n5c6ccccc6c6c7c(ccc65)sc5ccccc57)cc4)cccc32)cc1 ; c1ccc(-n2c3ccccc3c3c([Si](c4ccccc4)(c4ccccc4)c4ccc(-n5c6ccccc6c6c7c8ccccc8n(-c8ccccc8)c7ccc65)cc4)cccc32)cc1 ; CC1(C)c2ccccc2-c2c1ccc1c2c2ccccc2n1-c1ccc([Si](c2ccccc2)(c2ccccc2)c2cccc3c2c2ccccc2n3-c2ccccc2)cc1]

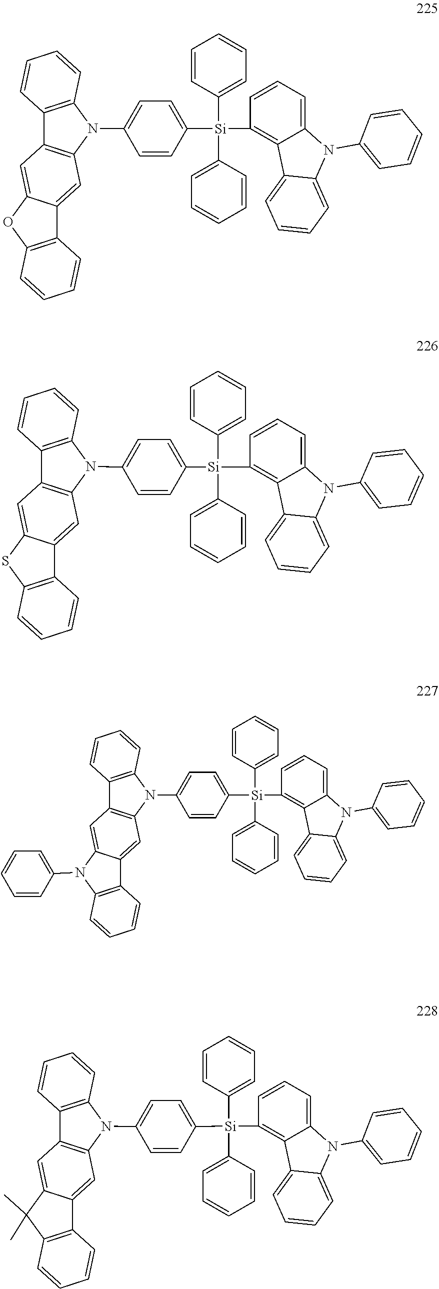 [SMILES: c1ccc(-n2c3ccccc3c3c([Si](c4ccccc4)(c4ccccc4)c4ccc(-n5c6ccccc6c6cc7oc8ccccc8c7cc65)cc4)cccc32)cc1 ; Cc1ccc(-n2c3ccccc3c3c([Si](c4ccccc4)(c4ccccc4)c4ccc(-n5c6ccccc6c6cc7sc8ccccc8c7cc65)cc4)cccc32)cc1 ; Cc1ccc(-n2c3ccccc3c3c([Si](c4ccccc4)(c4ccccc4)c4ccc(-n5c6ccccc6c6cc7c(cc65)c5ccccc5n7-c5ccccc5)cc4)cccc32)cc1 ; CC1(C)c2ccccc2-c2cc3c(cc21)c1ccccc1n3-c1ccc([Si](c2ccccc2)(c2ccccc2)c2cccc3c2c2ccccc2n3-c2ccccc2)cc1]

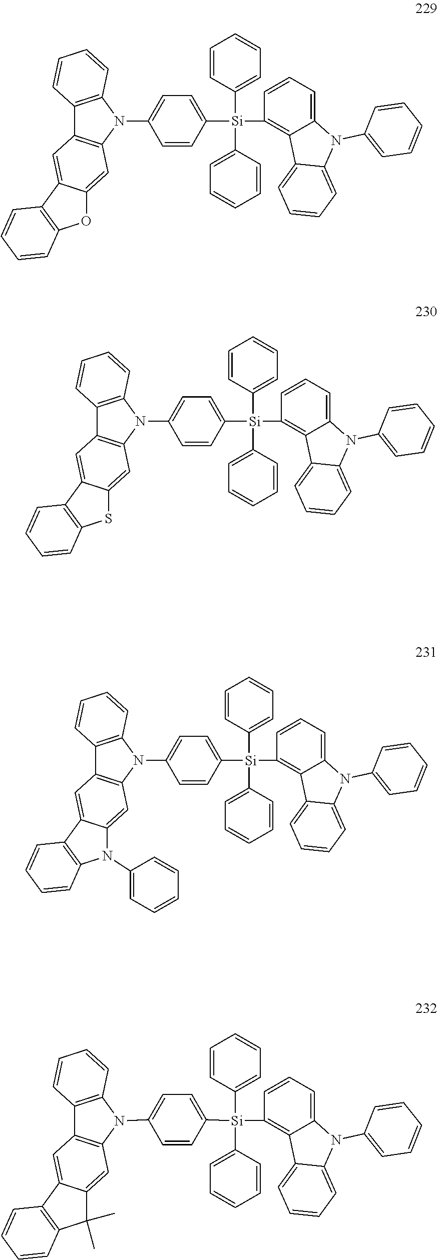 [SMILES: Cc1ccc(-n2c3ccccc3c3c([Si](c4ccccc4)(c4ccccc4)c4ccc(-n5c6ccccc6c6cc7c(cc65)oc5ccccc57)cc4)cccc32)cc1 ; Cc1ccc(-n2c3ccccc3c3c([Si](c4ccccc4)(c4ccccc4)c4ccc(-n5c6ccccc6c6cc7c(cc65)sc5ccccc57)cc4)cccc32)cc1 ; Cc1ccc(-n2c3ccccc3c3c([Si](c4ccccc4)(c4ccccc4)c4ccc(-n5c6ccccc6c6cc7c8ccccc8n(-c8ccccc8)c7cc65)cc4)cccc32)cc1 ; CC1(C)c2ccccc2-c2cc3c4ccccc4n(-c4ccc([Si](c5ccccc5)(c5ccccc5)c5cccc6c5c5ccccc5n6-c5ccccc5)cc4)c3cc21]

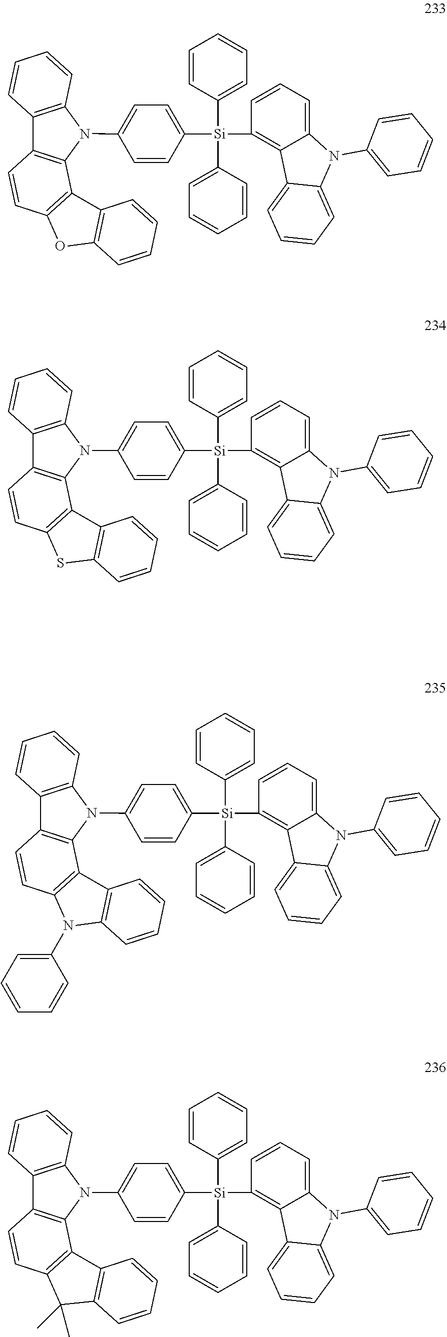 [SMILES: c1ccc(-n2c3ccccc3c3c([Si](c4ccccc4)(c4ccccc4)c4ccc(-n5c6ccccc6c6ccc7oc8ccccc8c7c65)cc4)cccc32)cc1 ; c1ccc(-n2c3ccccc3c3c([Si](c4ccccc4)(c4ccccc4)c4ccc(-n5c6ccccc6c6ccc7sc8ccccc8c7c65)cc4)cccc32)cc1 ; c1ccc(-n2c3ccccc3c3c([Si](c4ccccc4)(c4ccccc4)c4ccc(-n5c6ccccc6c6ccc7c(c8ccccc8n7-c7ccccc7)c65)cc4)cccc32)cc1 ; CC1(C)c2ccccc2-c2c1ccc1c3ccccc3n(-c3ccc([Si](c4ccccc4)(c4ccccc4)c4cccc5c4c4ccccc4n5-c4ccccc4)cc3)c21]

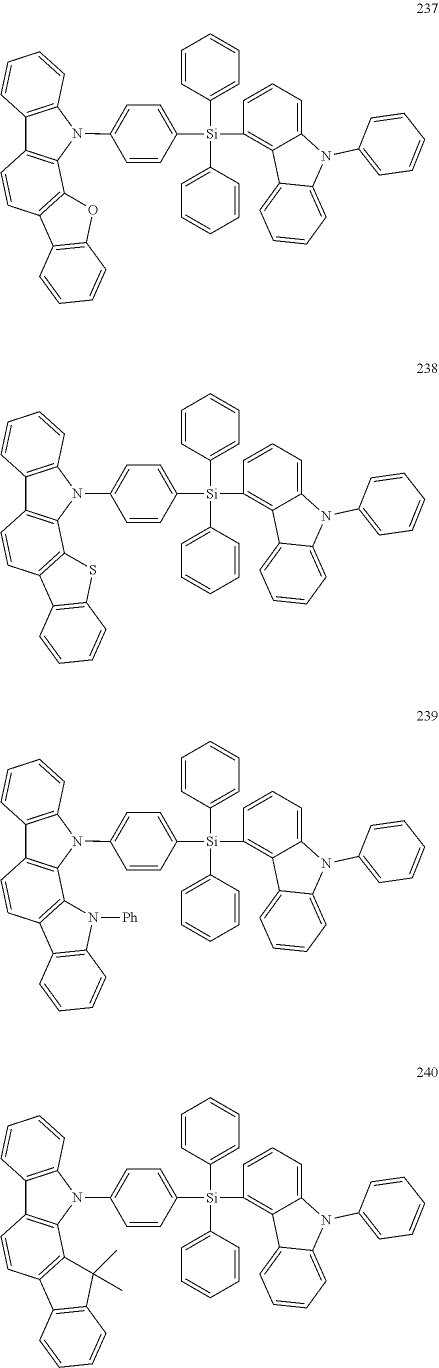 [SMILES: c1ccc(-n2c3ccccc3c3c([Si](c4ccccc4)(c4ccccc4)c4ccc(-n5c6ccccc6c6ccc7c8ccccc8oc7c65)cc4)cccc32)cc1 ; c1ccc(-n2c3ccccc3c3c([Si](c4ccccc4)(c4ccccc4)c4ccc(-n5c6ccccc6c6ccc7c8ccccc8sc7c65)cc4)cccc32)cc1 ; c1ccc(-n2c3ccccc3c3c([Si](c4ccccc4)(c4ccccc4)c4ccc(-n5c6ccccc6c6ccc7c8ccccc8n(-c8ccccc8)c7c65)cc4)cccc32)cc1 ; CC1(C)c2ccccc2-c2ccc3c4ccccc4n(-c4ccc([Si](c5ccccc5)(c5ccccc5)c5cccc6c5c5ccccc5n6-c5ccccc5)cc4)c3c21]

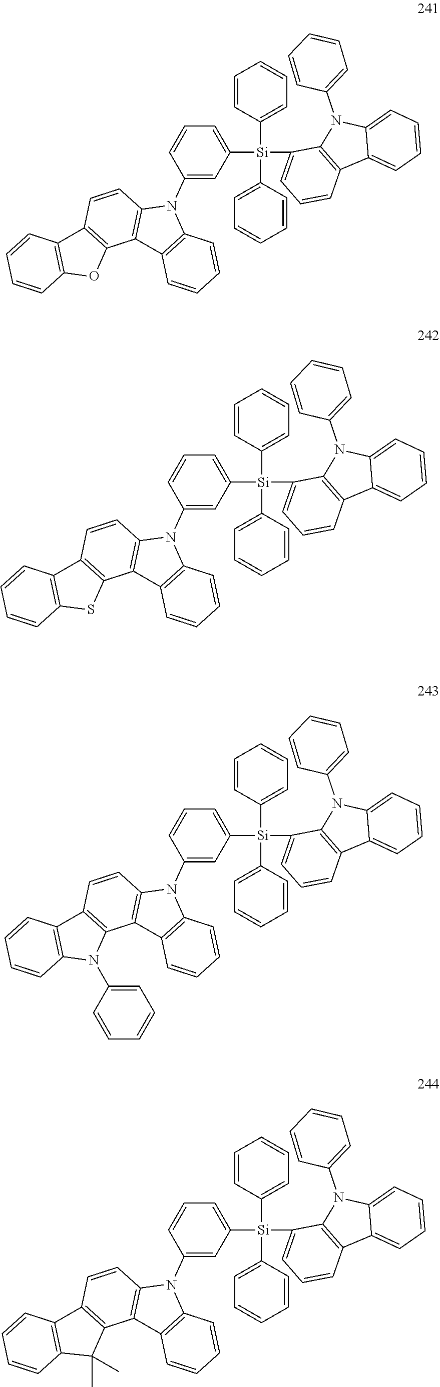 [SMILES: c1ccc(-n2c3ccccc3c3cccc([Si](c4ccccc4)(c4ccccc4)c4cccc(-n5c6ccccc6c6c7oc8ccccc8c7ccc65)c4)c32)cc1 ; c1ccc(-n2c3ccccc3c3cccc([Si](c4ccccc4)(c4ccccc4)c4cccc(-n5c6ccccc6c6c7sc8ccccc8c7ccc65)c4)c32)cc1 ; c1ccc(-n2c3ccccc3c3cccc([Si](c4ccccc4)(c4ccccc4)c4cccc(-n5c6ccccc6c6c5ccc5c7ccccc7n(-c7ccccc7)c56)c4)c32)cc1 ; CC1(C)c2ccccc2-c2ccc3c(c21)c1ccccc1n3-c1cccc([Si](c2ccccc2)(c2ccccc2)c2cccc3c4ccccc4n(-c4ccccc4)c23)c1]

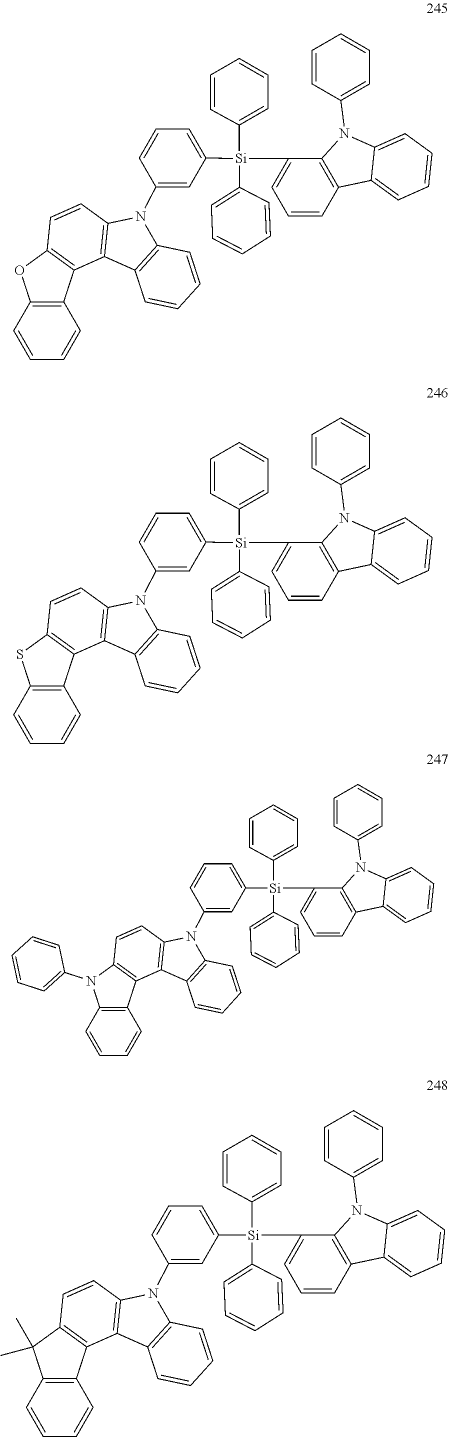 [SMILES: c1ccc(-n2c3ccccc3c3cccc([Si](c4ccccc4)(c4ccccc4)c4cccc(-n5c6ccccc6c6c7c(ccc65)oc5ccccc57)c4)c32)cc1 ; c1ccc(-n2c3ccccc3c3cccc([Si](c4ccccc4)(c4ccccc4)c4cccc(-n5c6ccccc6c6c7c(ccc65)sc5ccccc57)c4)c32)cc1 ; c1ccc(-n2c3ccccc3c3c4c5ccccc5n(-c5cccc([Si](c6ccccc6)(c6ccccc6)c6cccc7c8ccccc8n(-c8ccccc8)c67)c5)c4ccc32)cc1 ; CC1(C)c2ccccc2-c2c1ccc1c2c2ccccc2n1-c1cccc([Si](c2ccccc2)(c2ccccc2)c2cccc3c4ccccc4n(-c4ccccc4)c23)c1]

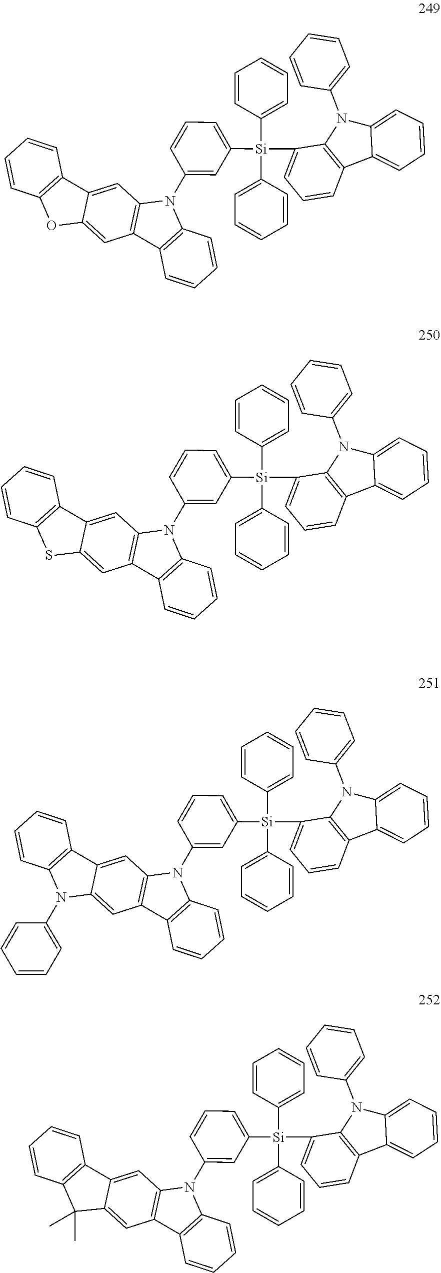 [SMILES: c1ccc(-n2c3ccccc3c3cccc([Si](c4ccccc4)(c4ccccc4)c4cccc(-n5c6ccccc6c6cc7oc8ccccc8c7cc65)c4)c32)cc1 ; c1ccc(-n2c3ccccc3c3cccc([Si](c4ccccc4)(c4ccccc4)c4cccc(-n5c6ccccc6c6cc7sc8ccccc8c7cc65)c4)c32)cc1 ; c1ccc(-n2c3ccccc3c3cc4c(cc32)c2ccccc2n4-c2cccc([Si](c3ccccc3)(c3ccccc3)c3cccc4c5ccccc5n(-c5ccccc5)c34)c2)cc1 ; CC1(C)c2ccccc2-c2cc3c(cc21)c1ccccc1n3-c1cccc([Si](c2ccccc2)(c2ccccc2)c2cccc3c4ccccc4n(-c4ccccc4)c23)c1]

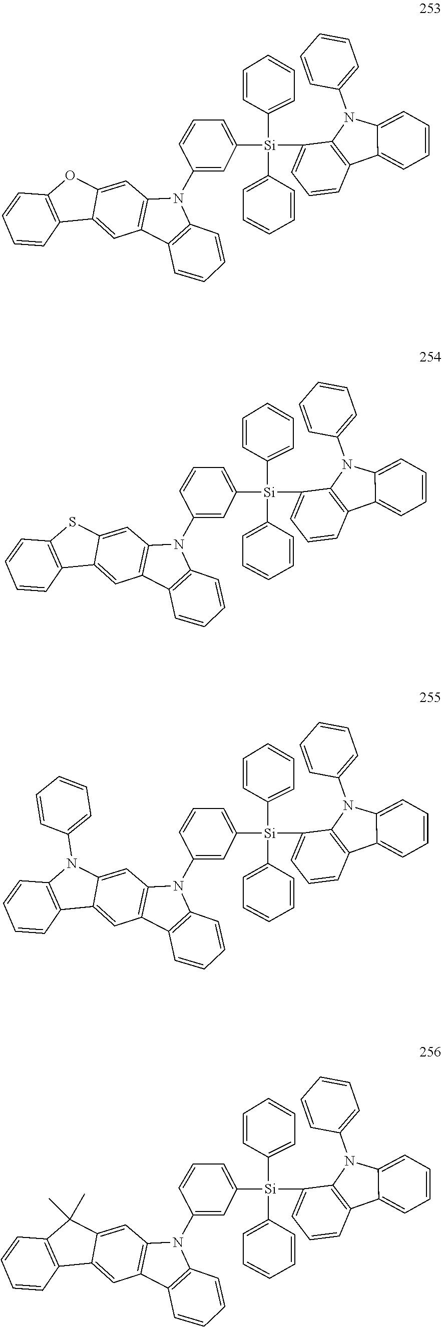 [SMILES: c1ccc(-n2c3ccccc3c3cccc([Si](c4ccccc4)(c4ccccc4)c4cccc(-n5c6ccccc6c6cc7c(cc65)oc5ccccc57)c4)c32)cc1 ; c1ccc(-n2c3ccccc3c3cccc([Si](c4ccccc4)(c4ccccc4)c4cccc(-n5c6ccccc6c6cc7c(cc65)sc5ccccc57)c4)c32)cc1 ; c1ccc(-n2c3ccccc3c3cc4c5ccccc5n(-c5cccc([Si](c6ccccc6)(c6ccccc6)c6cccc7c8ccccc8n(-c8ccccc8)c67)c5)c4cc32)cc1 ; CC1(C)c2ccccc2-c2cc3c4ccccc4n(-c4cccc([Si](c5ccccc5)(c5ccccc5)c5cccc6c7ccccc7n(-c7ccccc7)c56)c4)c3cc21]

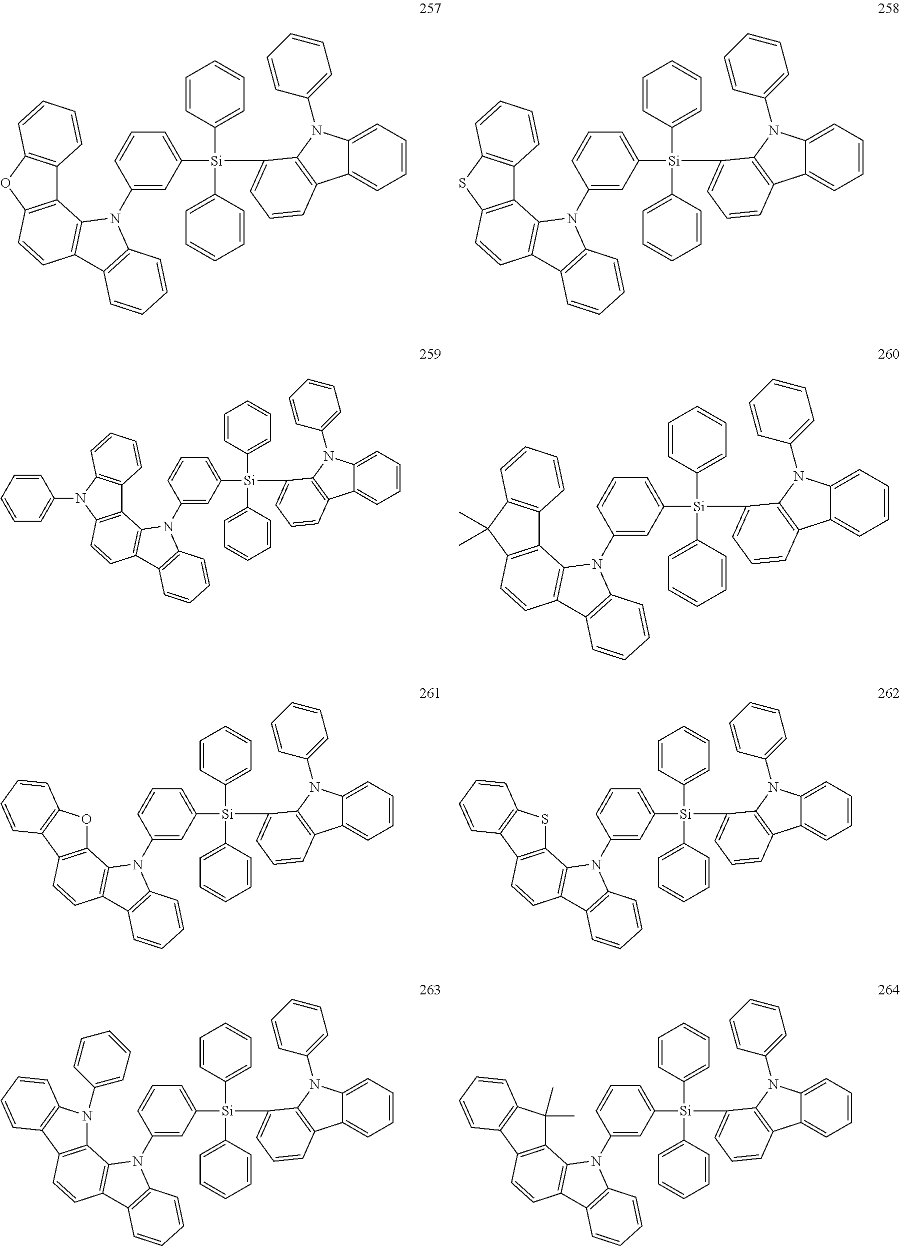

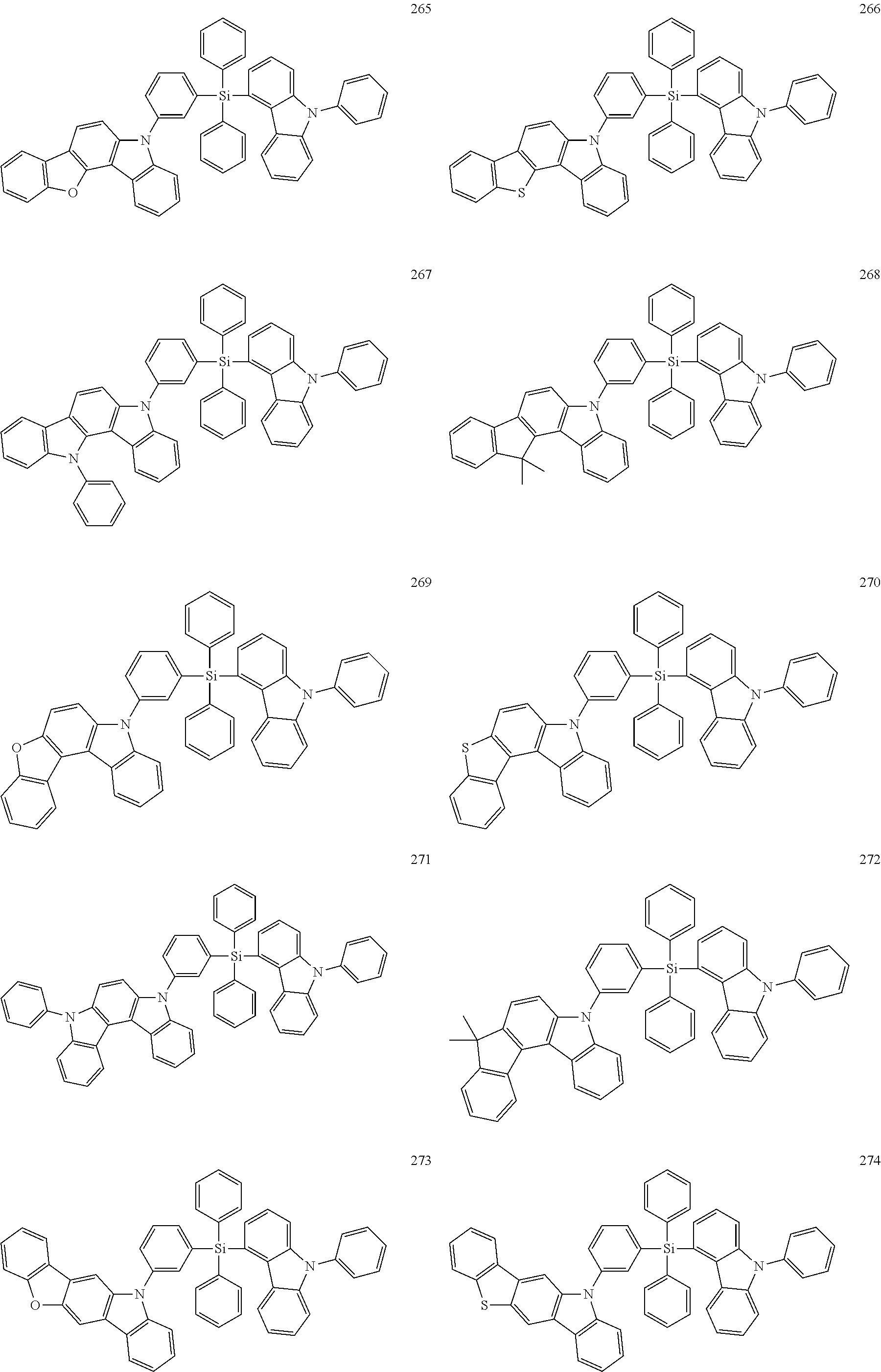

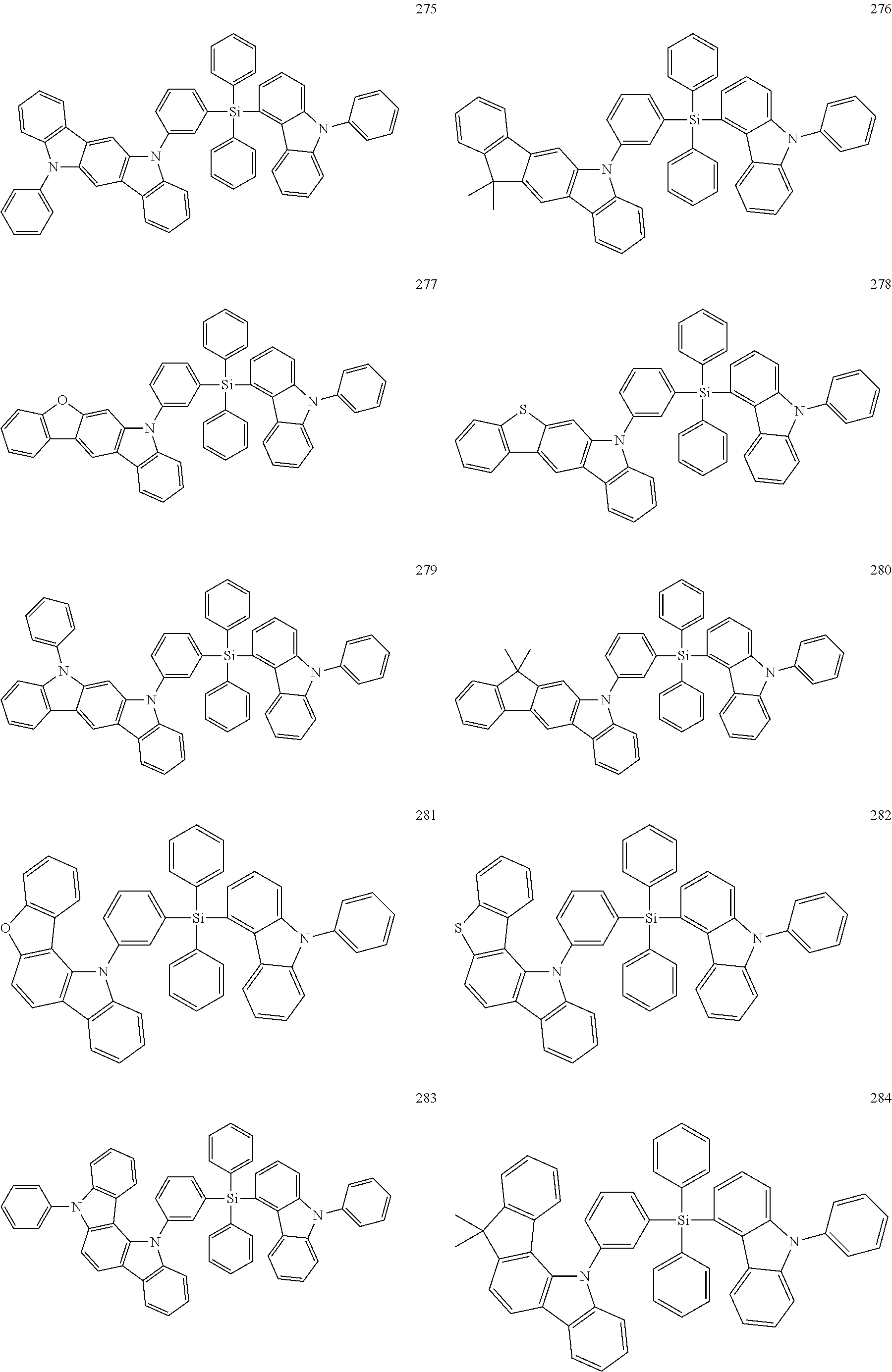

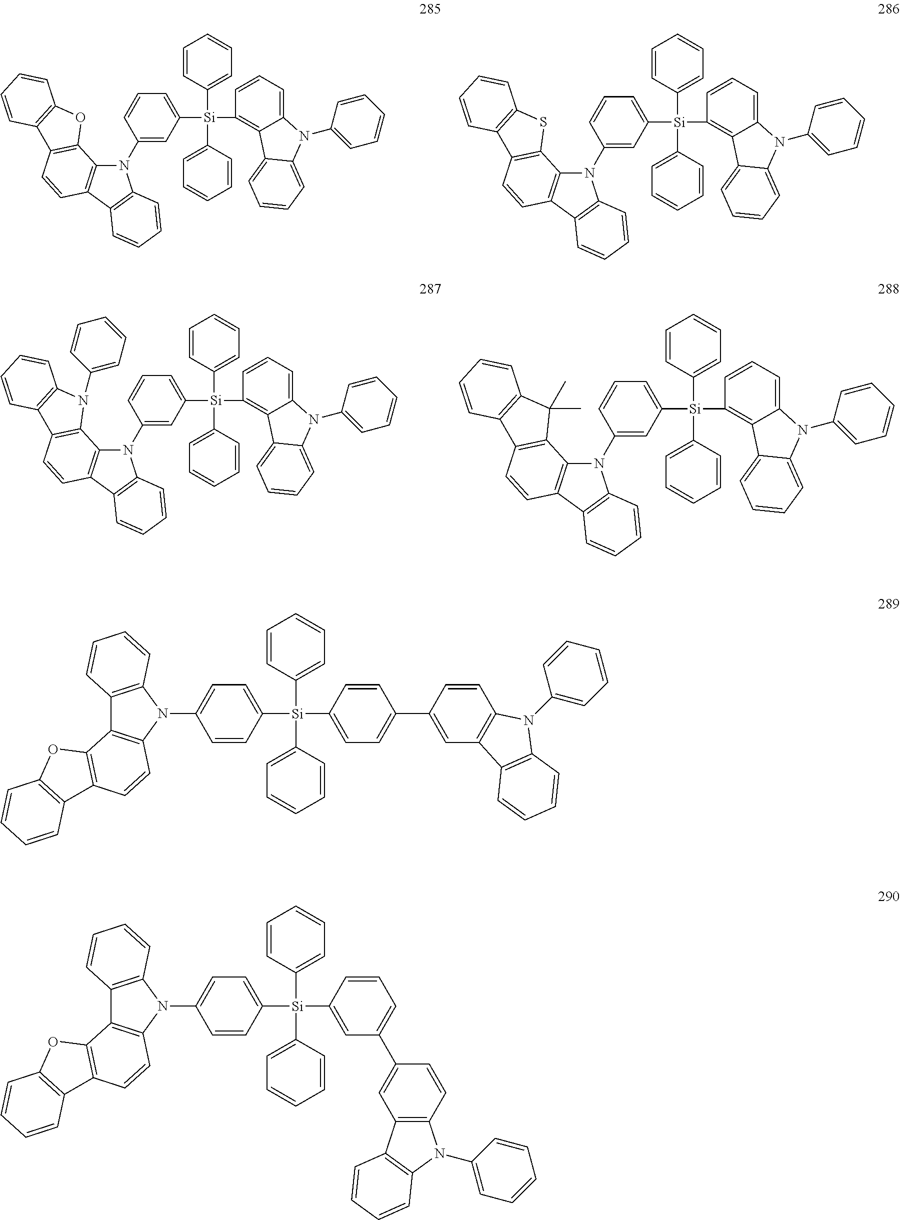

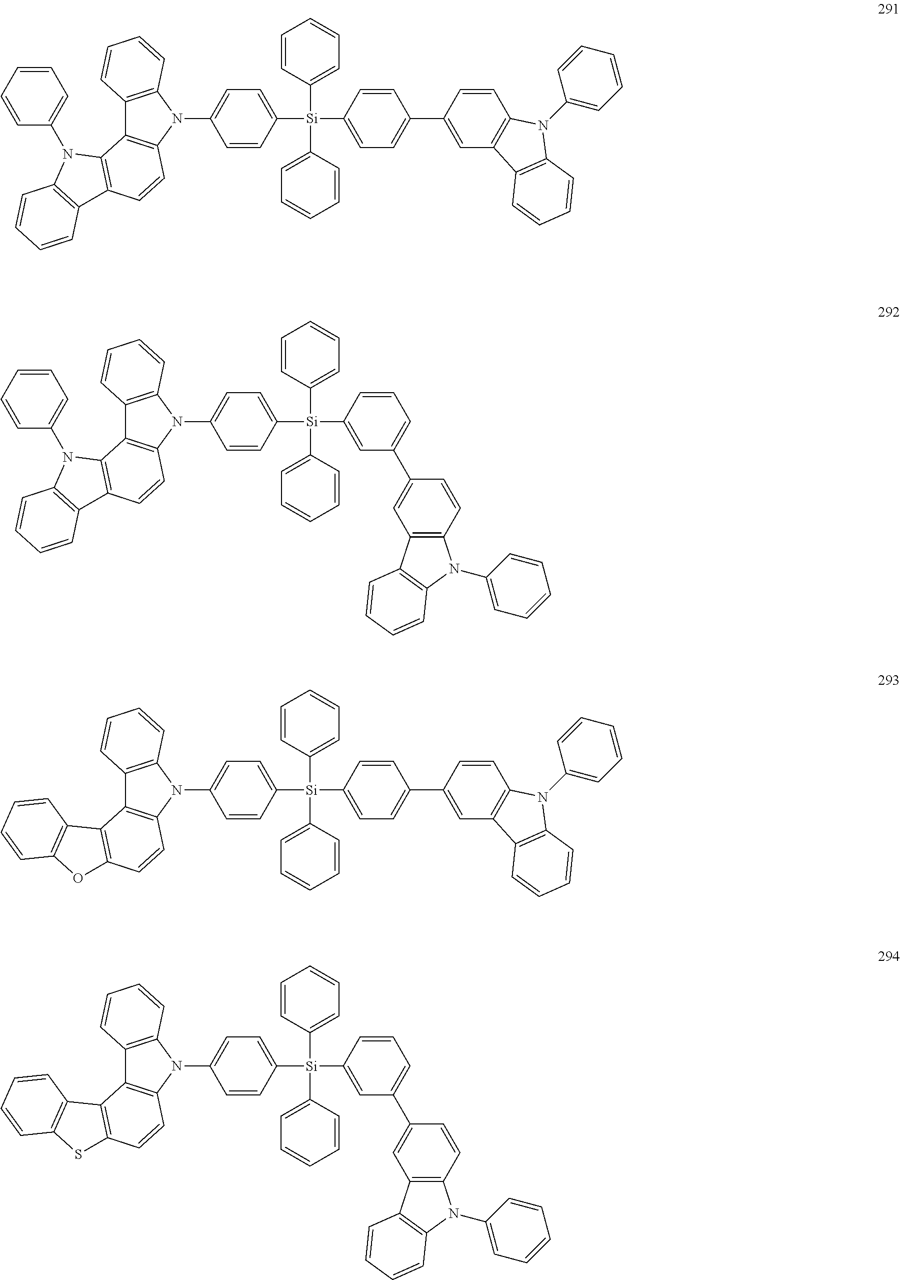 [SMILES: c1ccc(-n2c3ccccc3c3cc(-c4ccc([Si](c5ccccc5)(c5ccccc5)c5ccc(-n6c7ccccc7c7c6ccc6c8ccccc8n(-c8ccccc8)c67)cc5)cc4)ccc32)cc1 ; c1ccc(-n2c3ccccc3c3cc(-c4cccc([Si](c5ccccc5)(c5ccccc5)c5ccc(-n6c7ccccc7c7c6ccc6c8ccccc8n(-c8ccccc8)c67)cc5)c4)ccc32)cc1 ; c1ccc(-n2c3ccccc3c3cc(-c4ccc([Si](c5ccccc5)(c5ccccc5)c5ccc(-n6c7ccccc7c7c8c(ccc76)oc6ccccc68)cc5)cc4)ccc32)cc1 ; c1ccc(-n2c3ccccc3c3cc(-c4cccc([Si](c5ccccc5)(c5ccccc5)c5ccc(-n6c7ccccc7c7c8c(ccc76)sc6ccccc68)cc5)c4)ccc32)cc1]

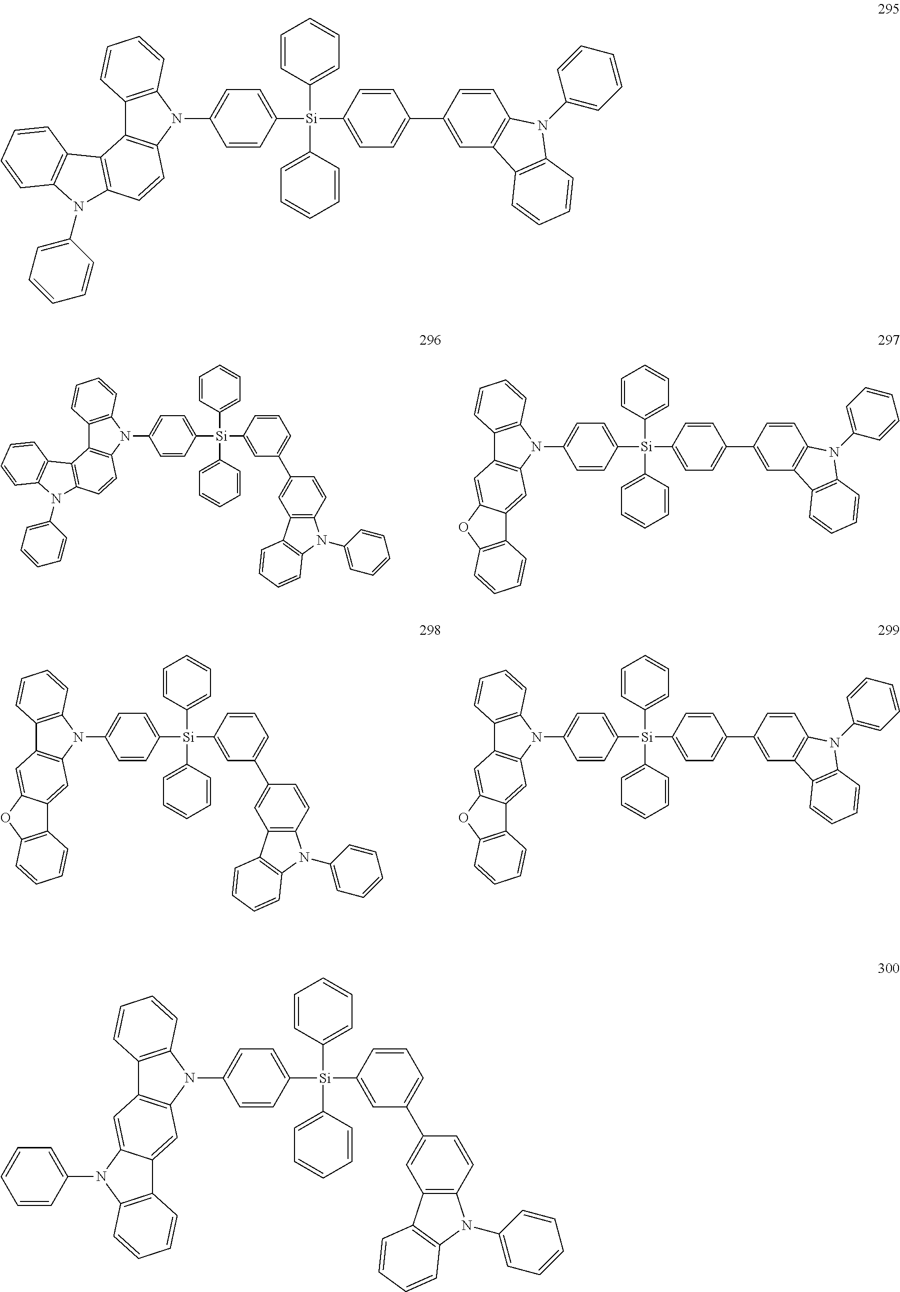

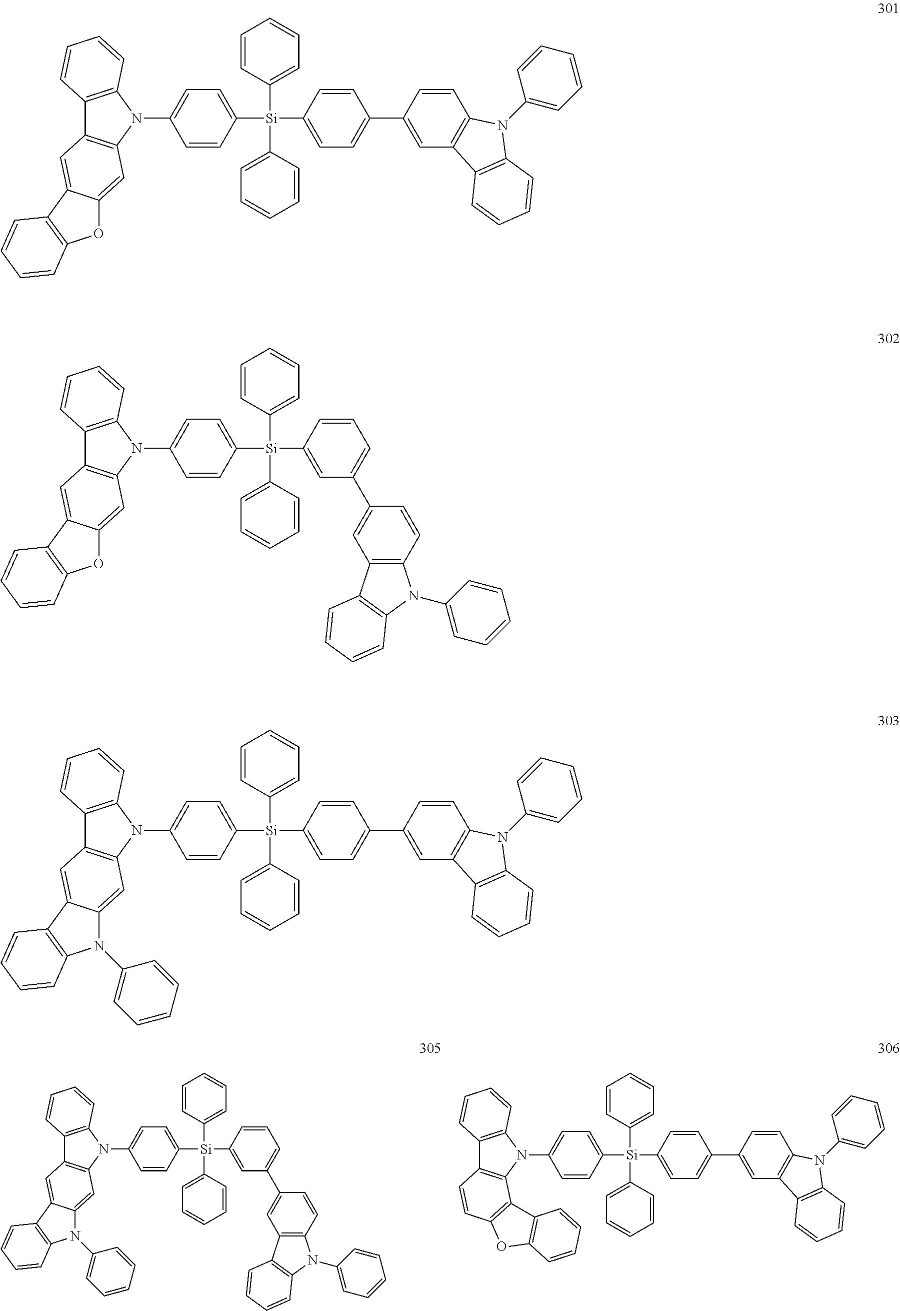

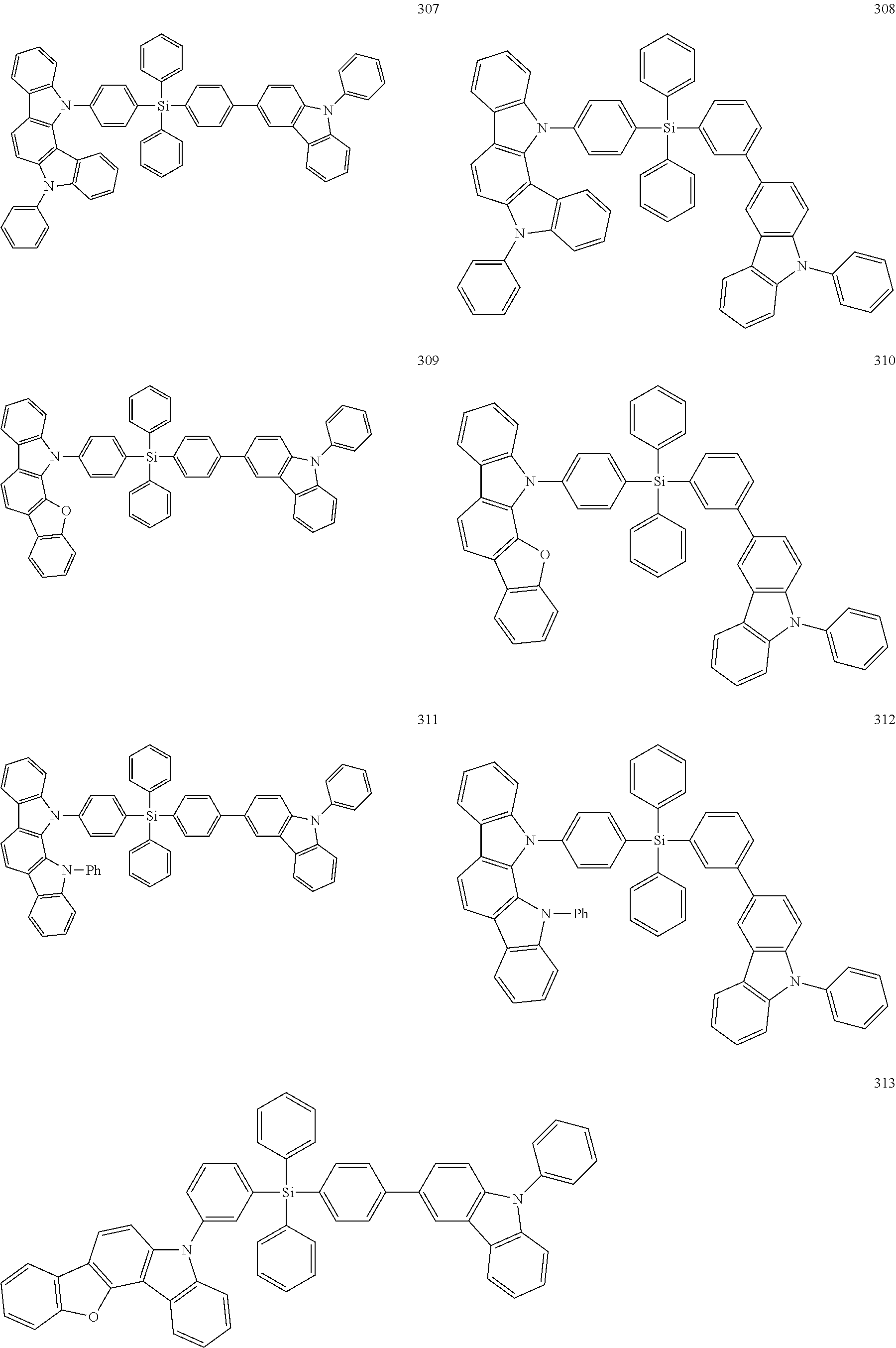

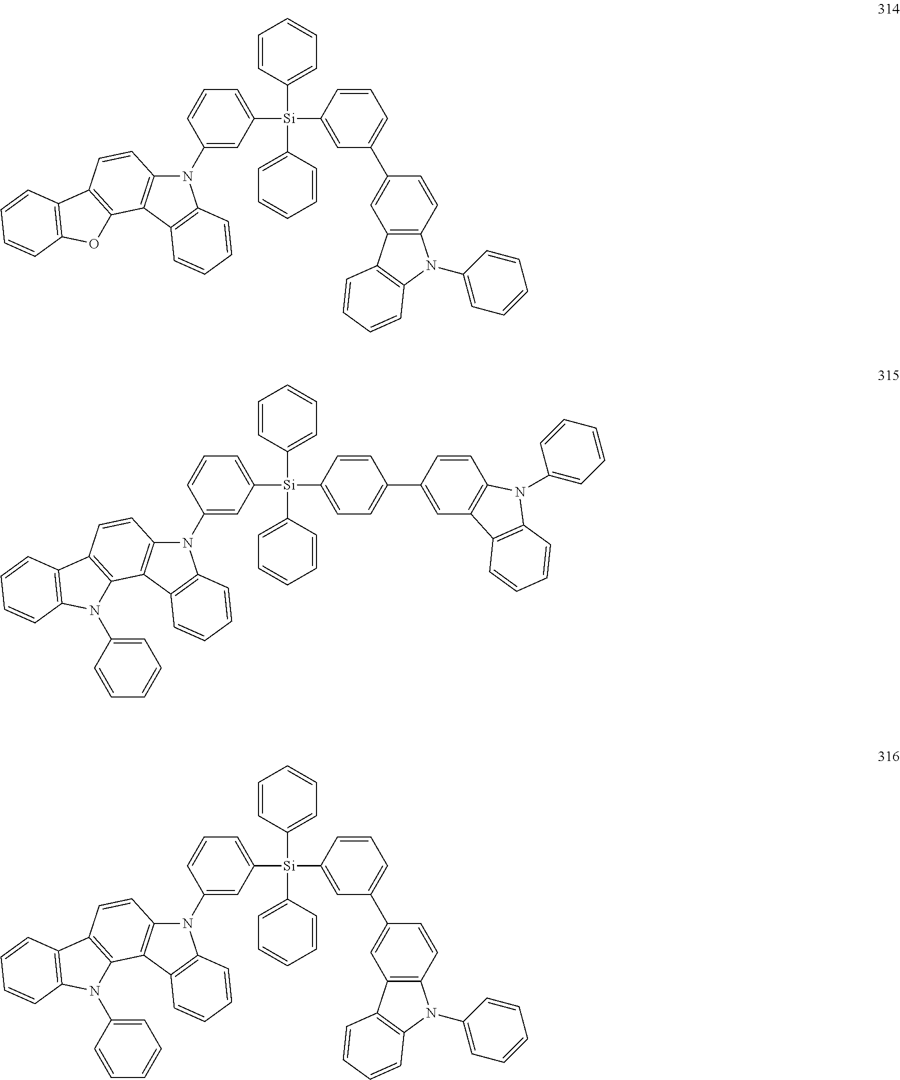 [SMILES: c1ccc(-n2c3ccccc3c3cc(-c4cccc([Si](c5ccccc5)(c5ccccc5)c5cccc(-n6c7ccccc7c7c8oc9ccccc9c8ccc76)c5)c4)ccc32)cc1 ; c1ccc(-n2c3ccccc3c3cc(-c4ccc([Si](c5ccccc5)(c5ccccc5)c5cccc(-n6c7ccccc7c7c6ccc6c8ccccc8n(-c8ccccc8)c67)c5)cc4)ccc32)cc1 ; c1ccc(-n2c3ccccc3c3cc(-c4cccc([Si](c5ccccc5)(c5ccccc5)c5cccc(-n6c7ccccc7c7c6ccc6c8ccccc8n(-c8ccccc8)c67)c5)c4)ccc32)cc1]

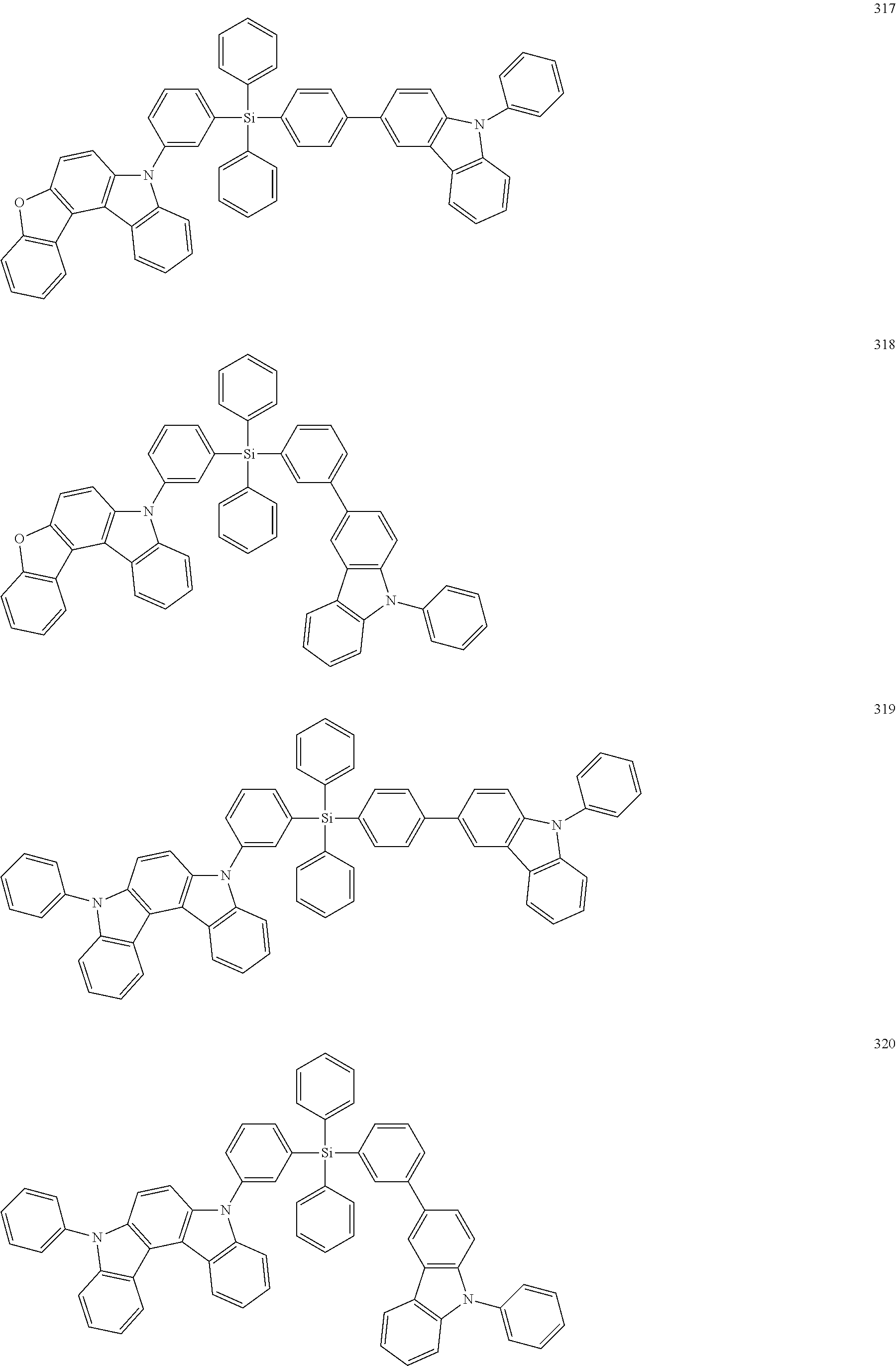 [SMILES: c1ccc(-n2c3ccccc3c3cc(-c4ccc([Si](c5ccccc5)(c5ccccc5)c5cccc(-n6c7ccccc7c7c8c(ccc76)oc6ccccc68)c5)cc4)ccc32)cc1 ; c1ccc(-n2c3ccccc3c3cc(-c4cccc([Si](c5ccccc5)(c5ccccc5)c5cccc(-n6c7ccccc7c7c8c(ccc76)oc6ccccc68)c5)c4)ccc32)cc1 ; c1ccc(-n2c3ccccc3c3cc(-c4ccc([Si](c5ccccc5)(c5ccccc5)c5cccc(-n6c7ccccc7c7c8c9ccccc9n(-c9ccccc9)c8ccc76)c5)cc4)ccc32)cc1 ; c1ccc(-n2c3ccccc3c3cc(-c4cccc([Si](c5ccccc5)(c5ccccc5)c5cccc(-n6c7ccccc7c7c8c9ccccc9n(-c9ccccc9)c8ccc76)c5)c4)ccc32)cc1]

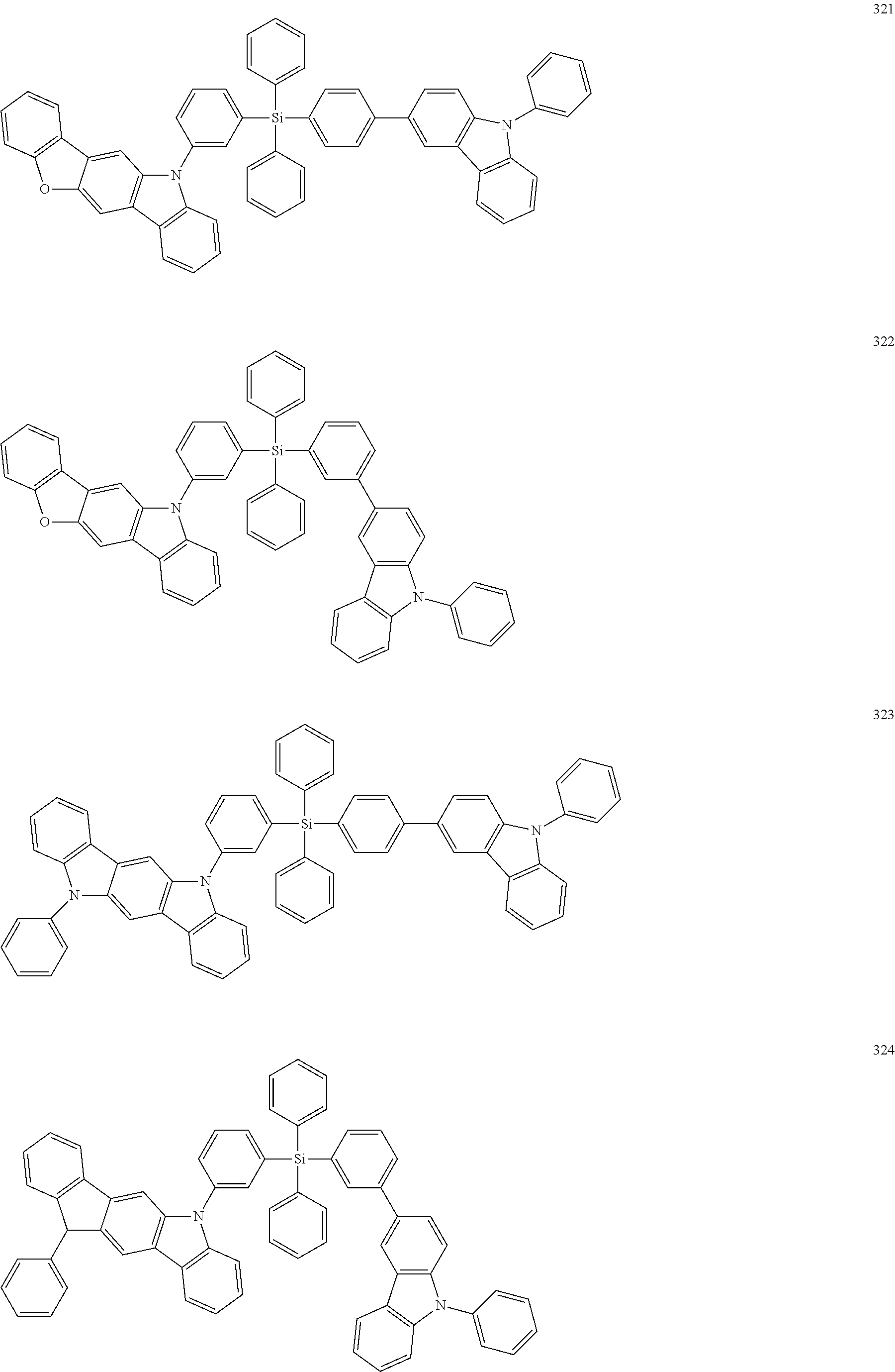 [SMILES: c1ccc(-n2c3ccccc3c3cc(-c4ccc([Si](c5ccccc5)(c5ccccc5)c5cccc(-n6c7ccccc7c7cc8oc9ccccc9c8cc76)c5)cc4)ccc32)cc1 ; c1ccc(-n2c3ccccc3c3cc(-c4cccc([Si](c5ccccc5)(c5ccccc5)c5cccc(-n6c7ccccc7c7cc8oc9ccccc9c8cc76)c5)c4)ccc32)cc1 ; c1ccc(-n2c3ccccc3c3cc(-c4ccc([Si](c5ccccc5)(c5ccccc5)c5cccc(-n6c7ccccc7c7cc8c(cc76)c6ccccc6n8-c6ccccc6)c5)cc4)ccc32)cc1 ; c1ccc(C2c3ccccc3-c3cc4c(cc32)c2ccccc2n4-c2cccc([Si](c3ccccc3)(c3ccccc3)c3cccc(-c4ccc5c(c4)c4ccccc4n5-c4ccccc4)c3)c2)cc1]

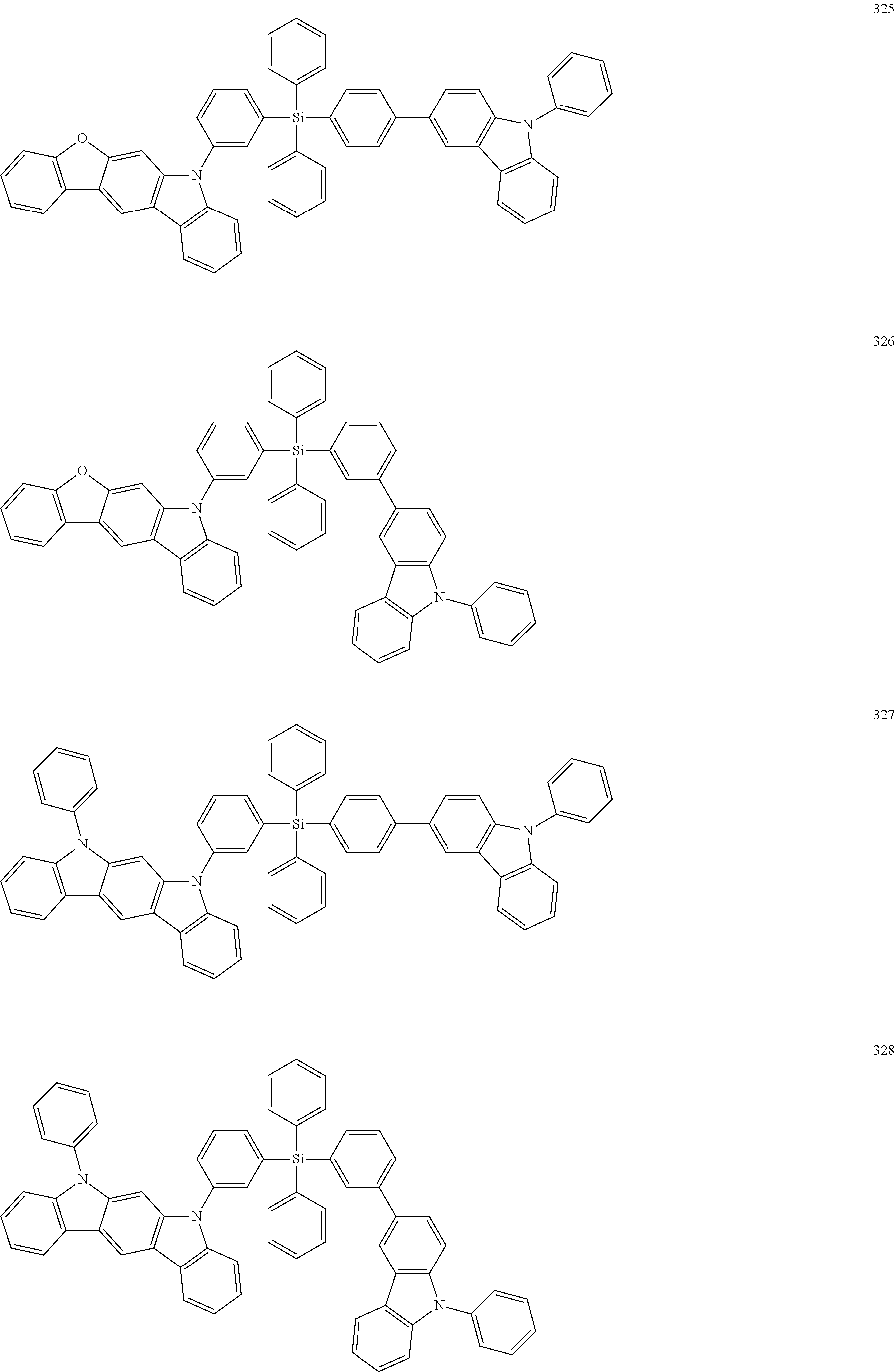 [SMILES: c1ccc(-n2c3ccccc3c3cc(-c4ccc([Si](c5ccccc5)(c5ccccc5)c5cccc(-n6c7ccccc7c7cc8c(cc76)oc6ccccc68)c5)cc4)ccc32)cc1 ; c1ccc(-n2c3ccccc3c3cc(-c4cccc([Si](c5ccccc5)(c5ccccc5)c5cccc(-n6c7ccccc7c7cc8c(cc76)oc6ccccc68)c5)c4)ccc32)cc1 ; c1ccc(-n2c3ccccc3c3cc(-c4ccc([Si](c5ccccc5)(c5ccccc5)c5cccc(-n6c7ccccc7c7cc8c9ccccc9n(-c9ccccc9)c8cc76)c5)cc4)ccc32)cc1 ; c1ccc(-n2c3ccccc3c3cc(-c4cccc([Si](c5ccccc5)(c5ccccc5)c5cccc(-n6c7ccccc7c7cc8c9ccccc9n(-c9ccccc9)c8cc76)c5)c4)ccc32)cc1]

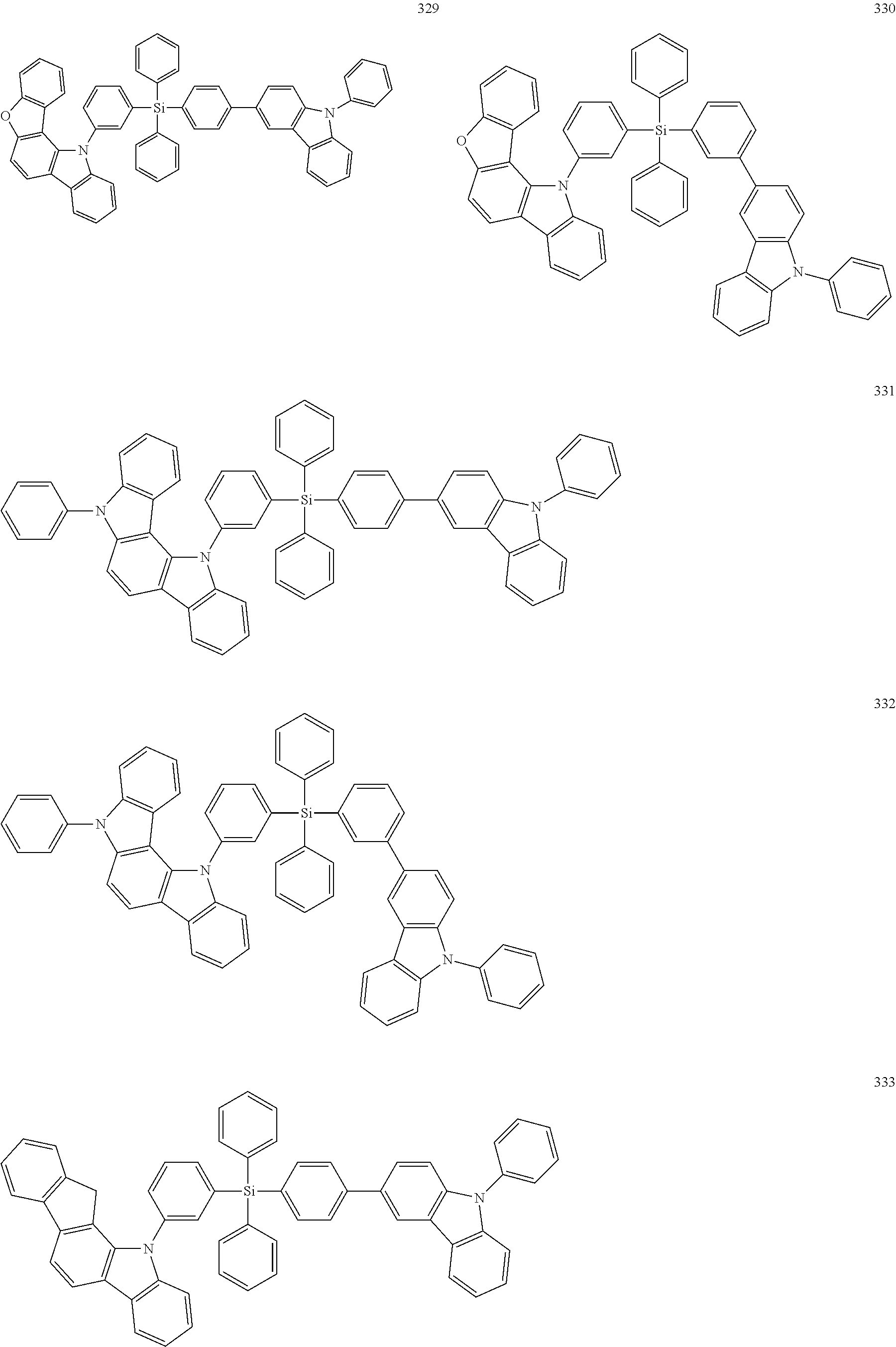

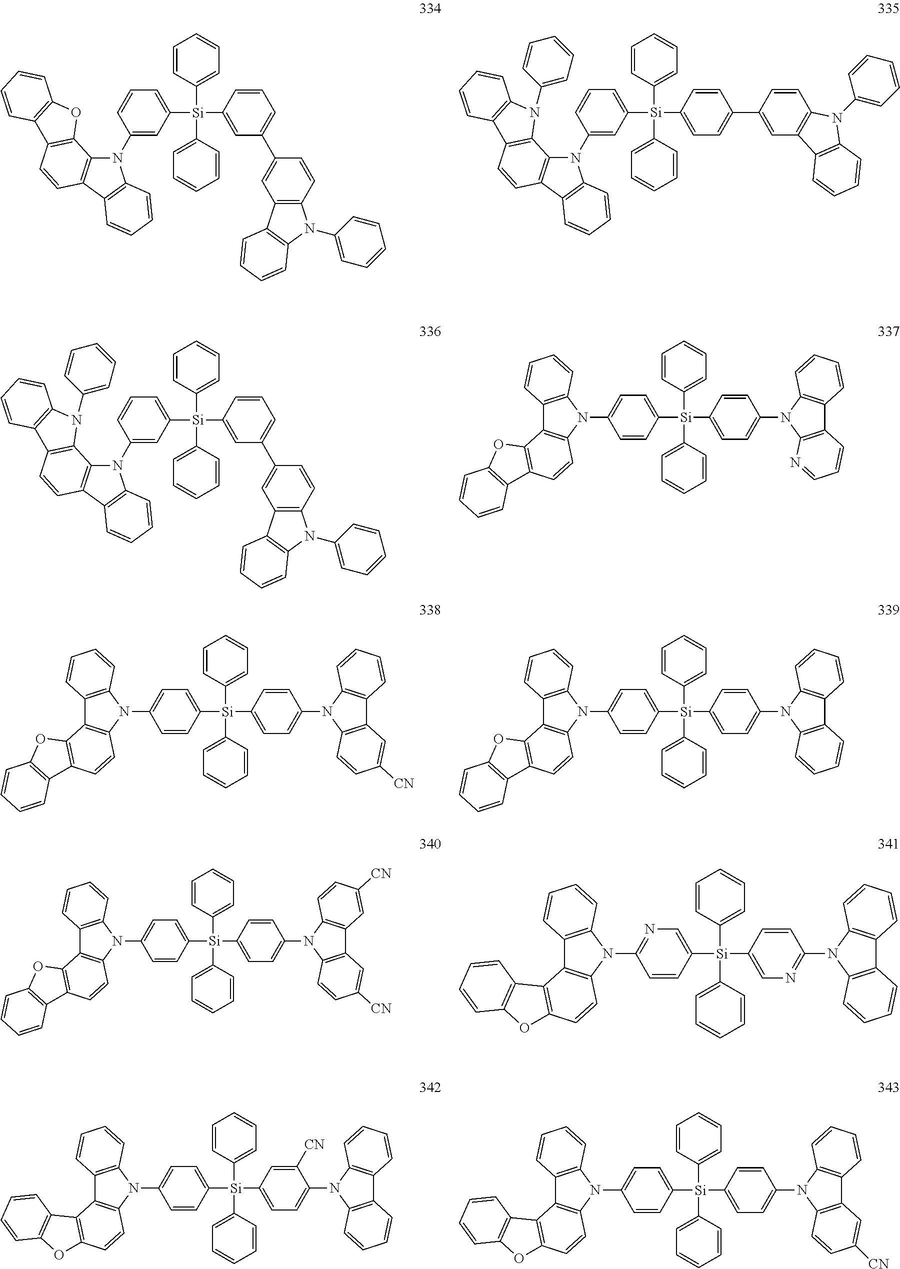

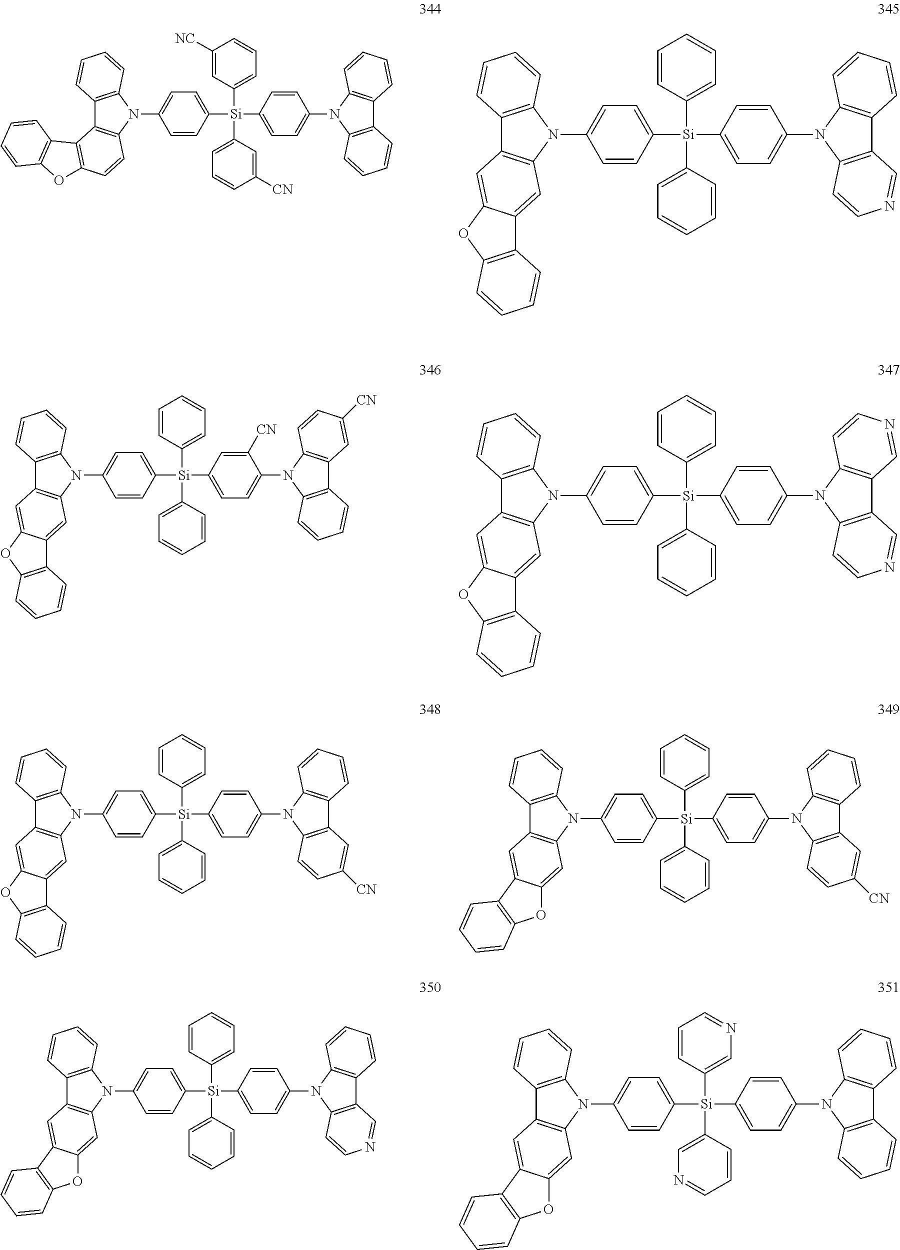

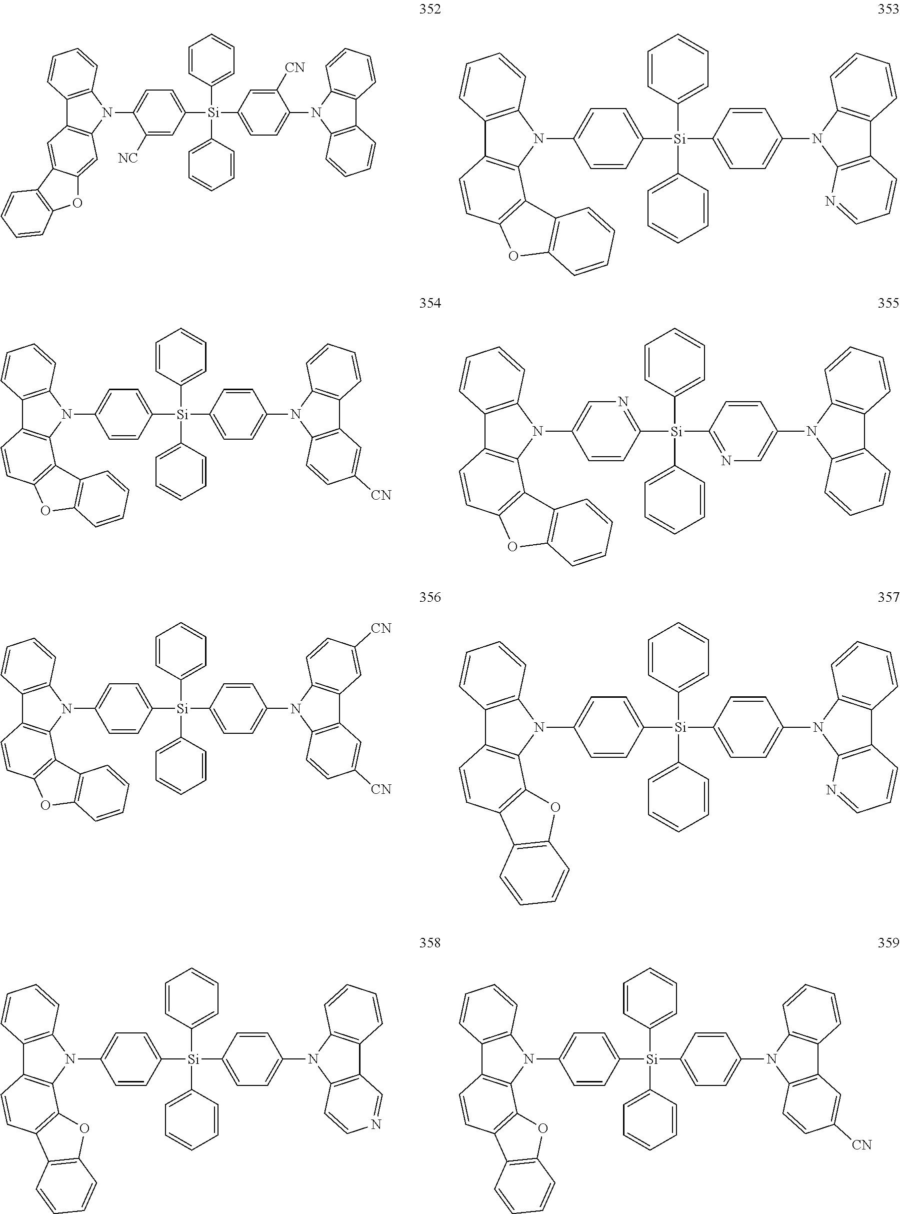

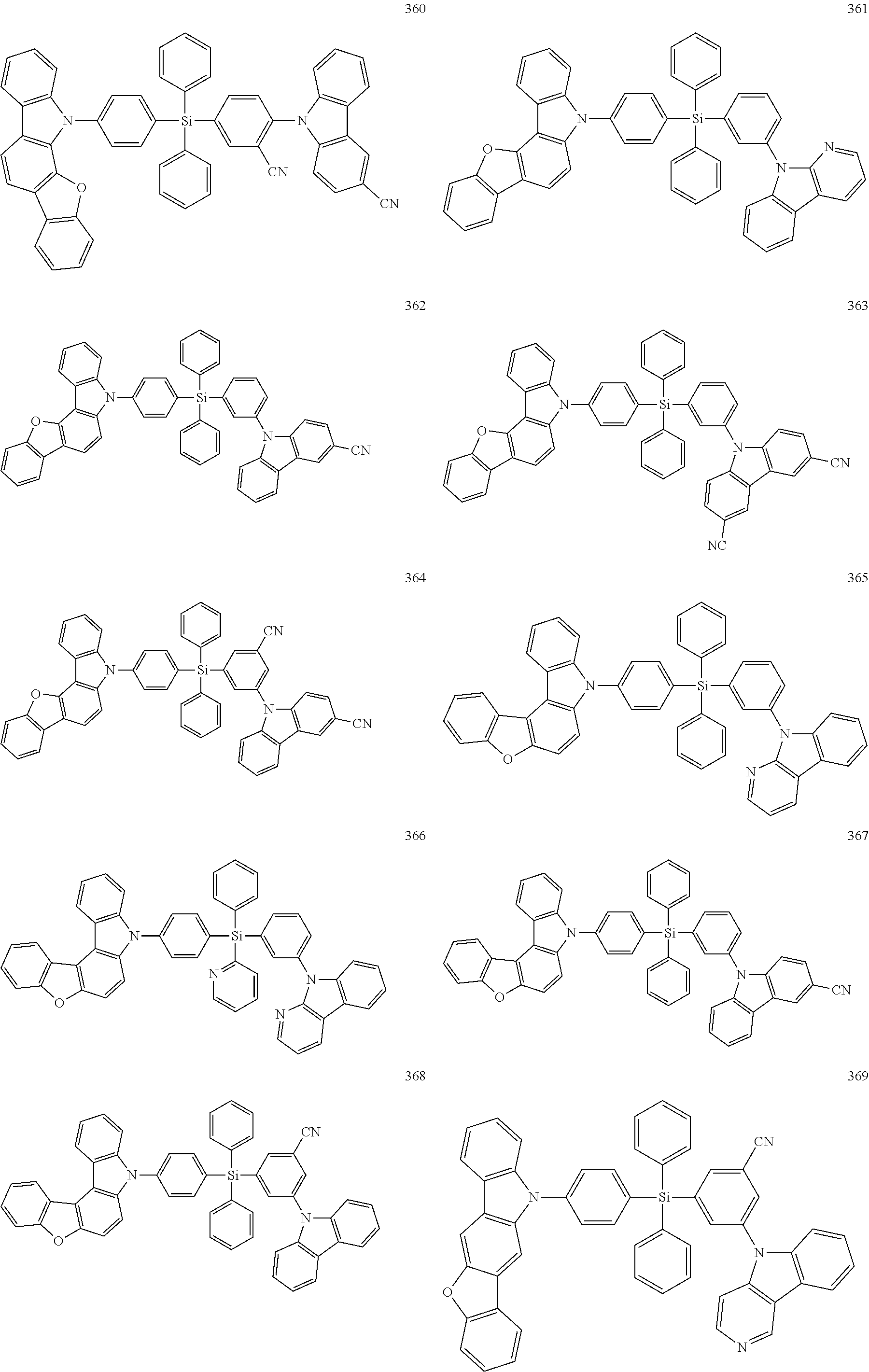

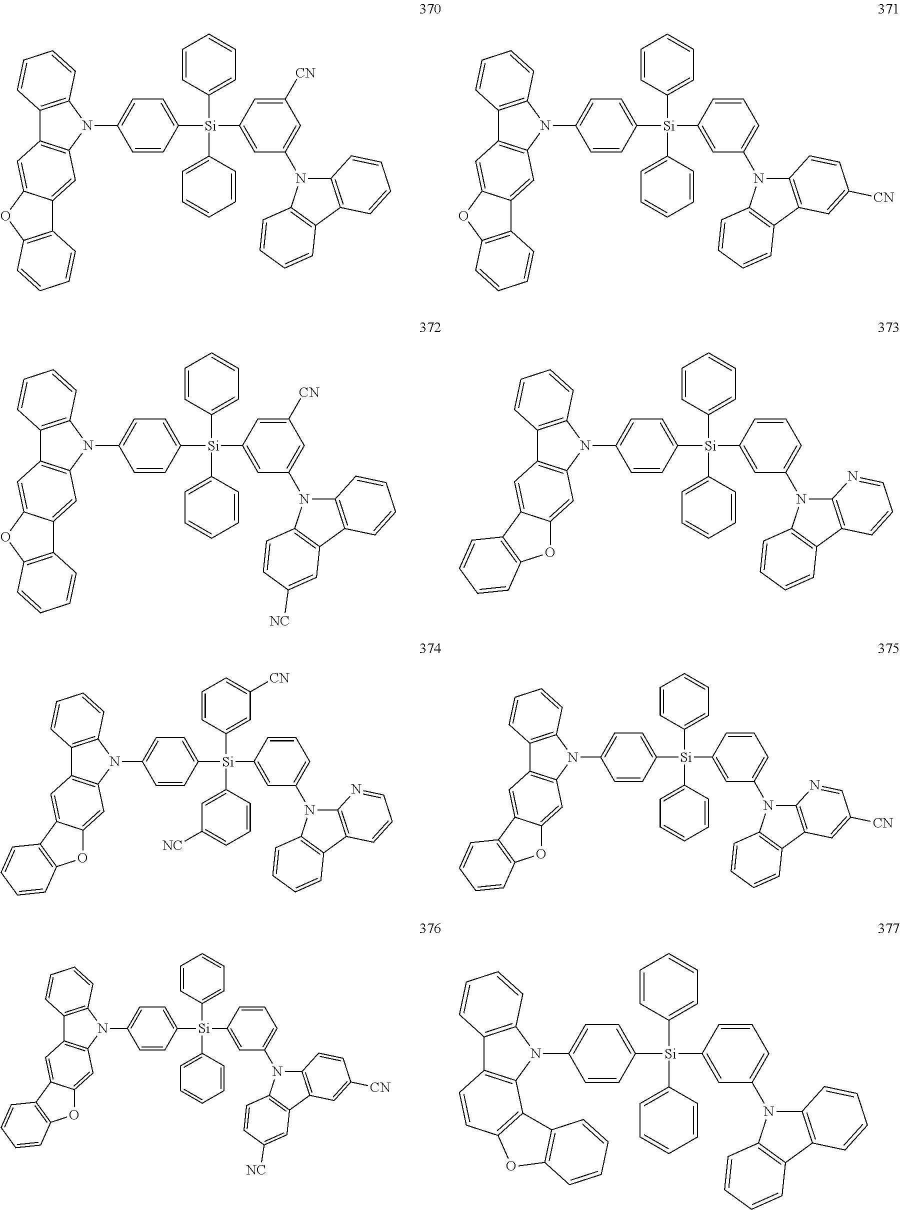

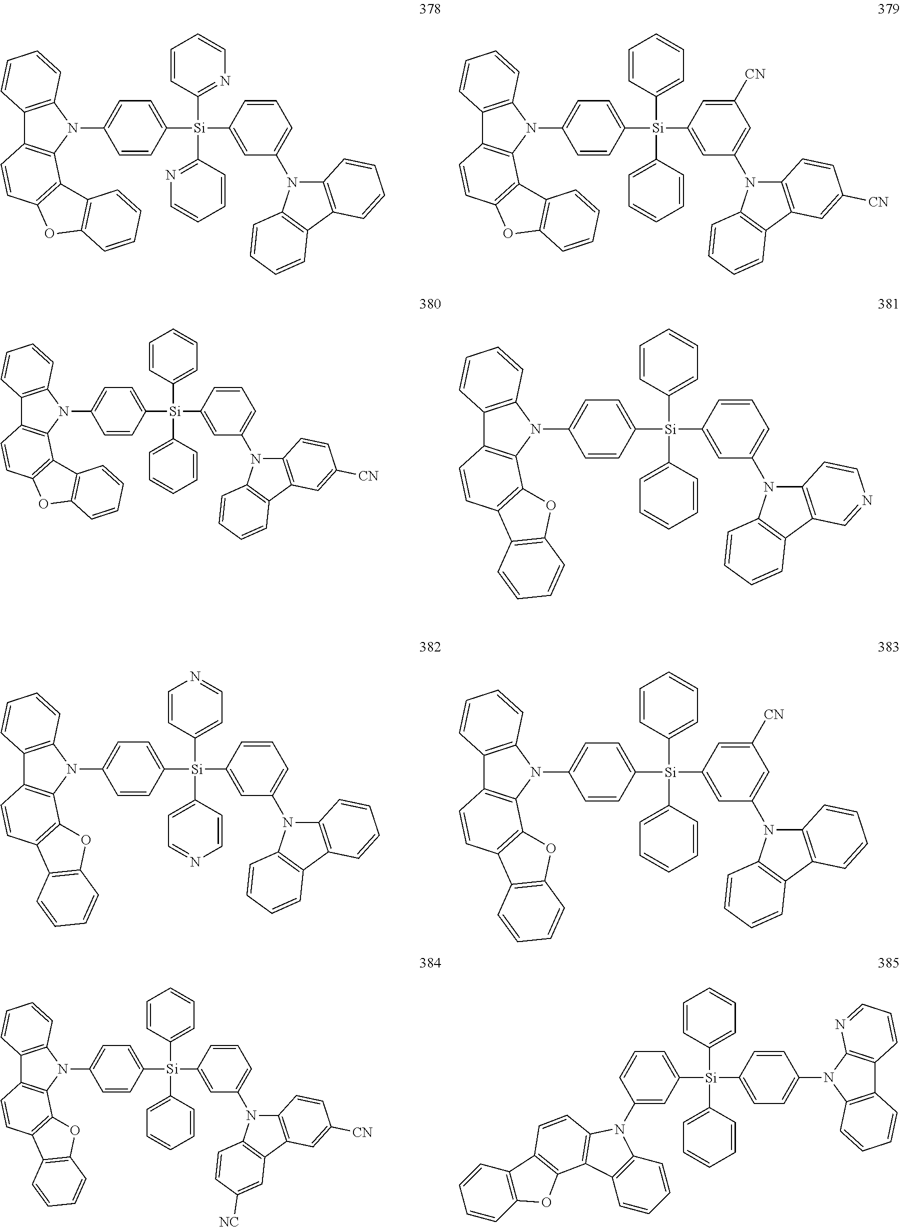

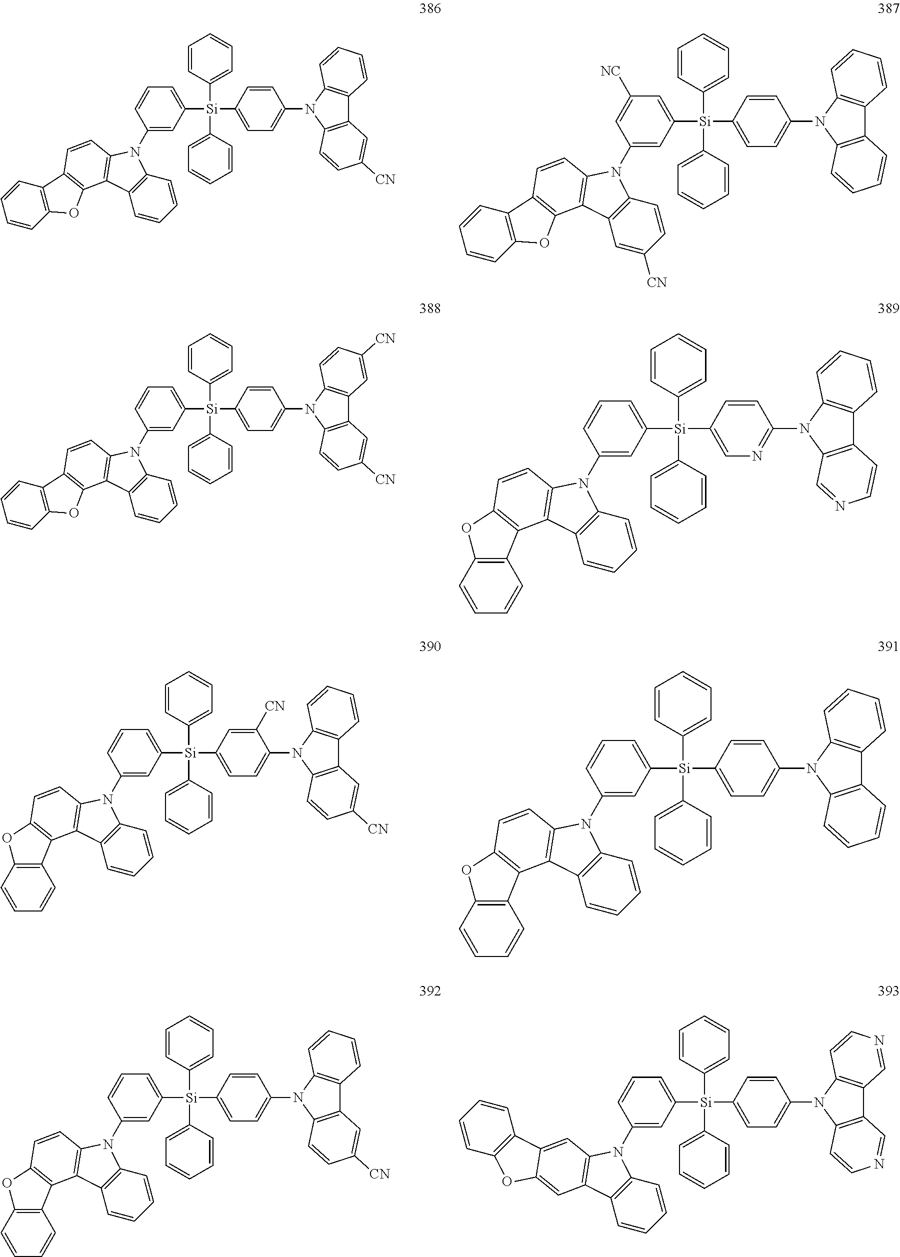

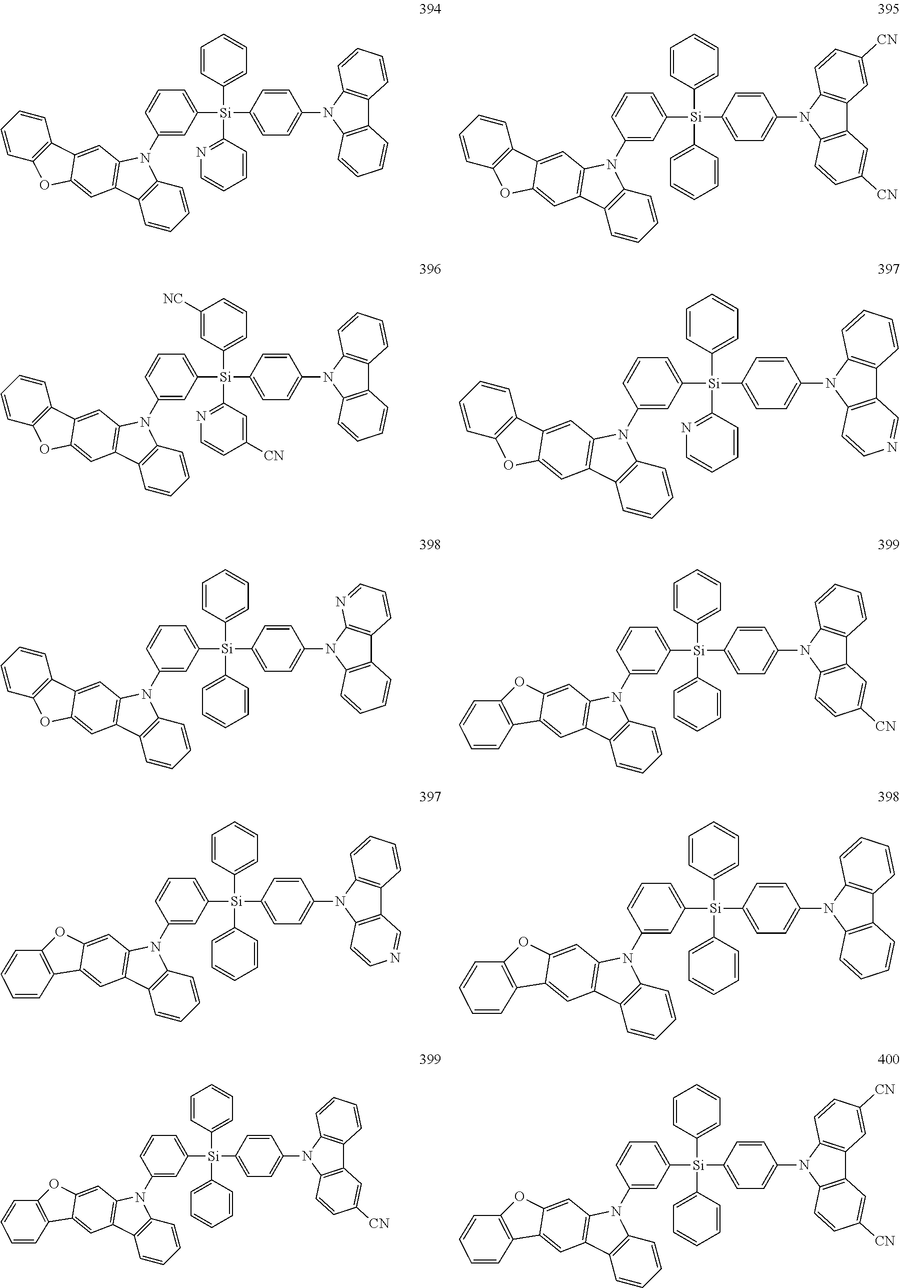

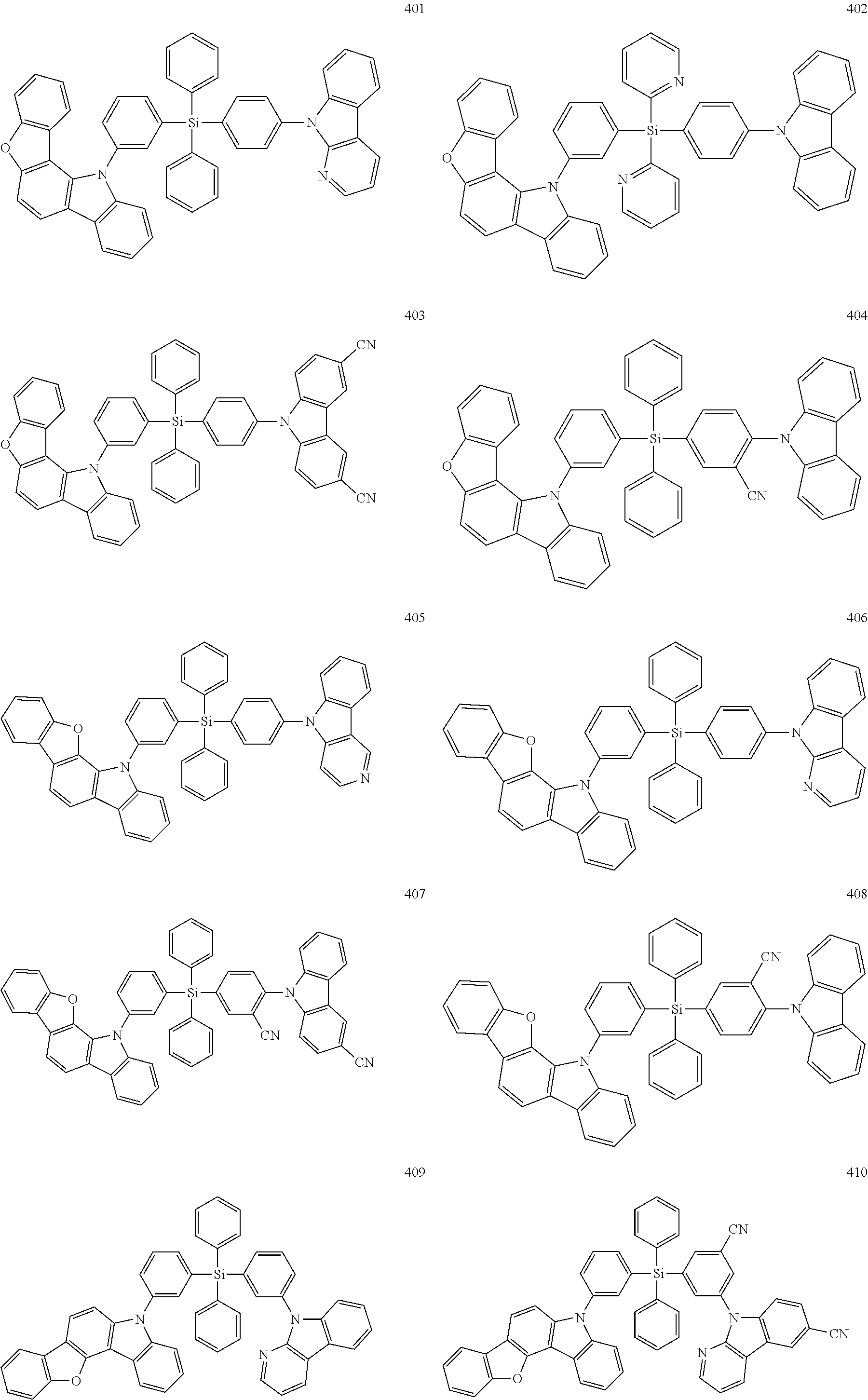

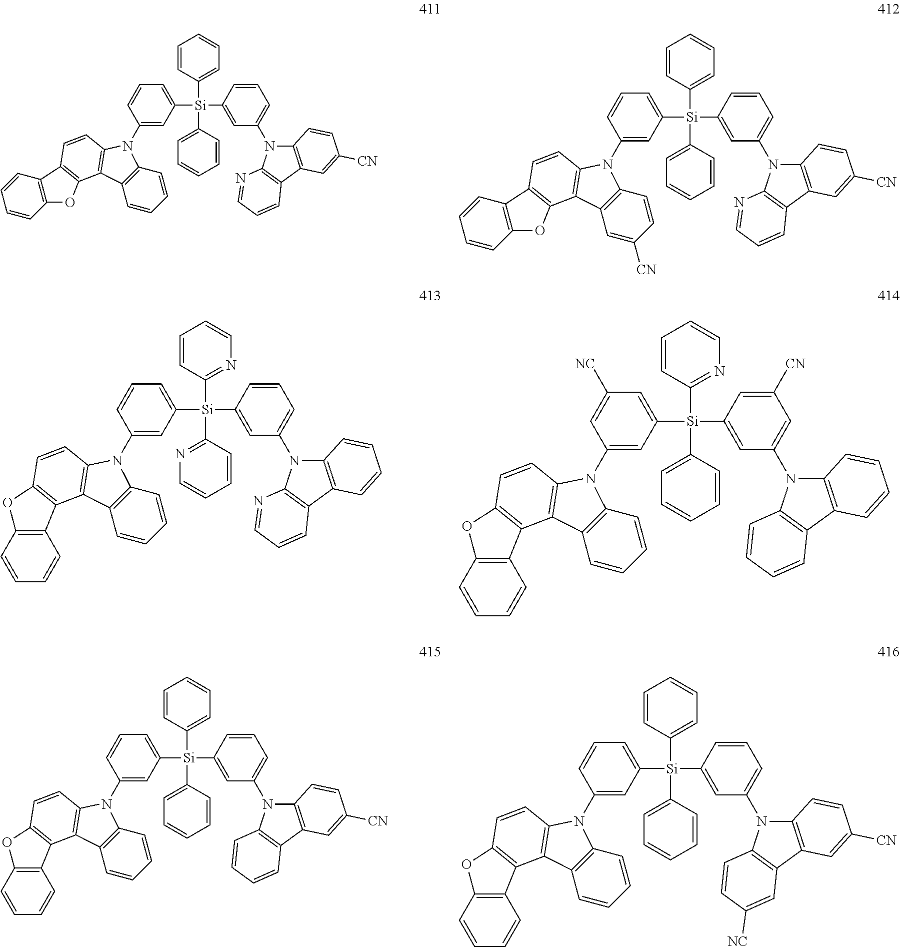

Ph in Compounds 1 to 432 is a phenyl group.

In an embodiment, the hole-transporting host may include o-CBP, or mCP:

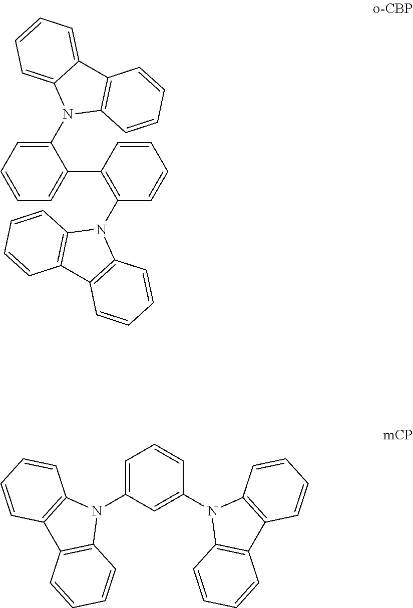 [SMILES: c1ccc(-n2c3ccccc3c3ccccc32)c(-c2ccccc2-n2c3ccccc3c3ccccc32)c1 ; c1cc(-n2c3ccccc3c3ccccc32)cc(-n2c3ccccc3c3ccccc32)c1]

In an embodiment, the host may be a fluorescent host, and the fluorescent host may be represented by, for example, any one of Formulae FH-1 to FH-4.

In an embodiment, the fluorescent host may be represented by Formula FH-1:

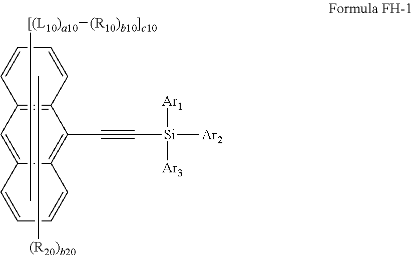

In Formula FH-1,

Ar₁ to Ar₃ may each independently be a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

at least one of Ar₁ to Ar₃ may each independently be a substituted or unsubstituted C₁-C₆₀ alkyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group,

L₁₀ is a substituted or unsubstituted C₅-C₃₀ carbocyclic group or a substituted or unsubstituted C₁-C₃₀ heterocyclic group,

a10 is an integer from 0 to 3, and when a10 is 2 or more, two or more of L₁₀₍ₛ₎ may be identical to or different from each other,

R₁₀ and R₂₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

b10 and b20 may each independently an integer from 1 to 8,

wherein, when b10 is 2 or more, two or more of R₁₀(s) may be identical to or different from each other, and when b20 is 2 or more, two or more of R₂₀(s) may be identical to or different from each other,

c10 may be an integer from 1 to 9, and

when c10 is 2 or more, two or more of -[(L₁₀)ₐ₁₀-(R₁₀)_{b10}](s) may be identical to each other or different from each other.

In an embodiment, the fluorescent host represented by Formula FH-1 may be Group FH1, but embodiments of the present disclosure are not limited thereto:

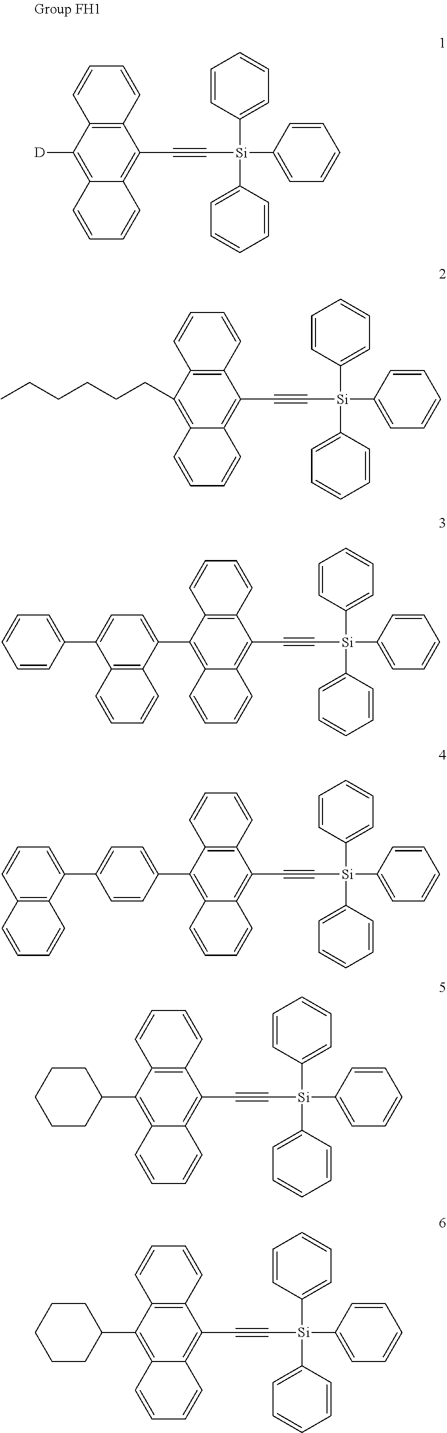

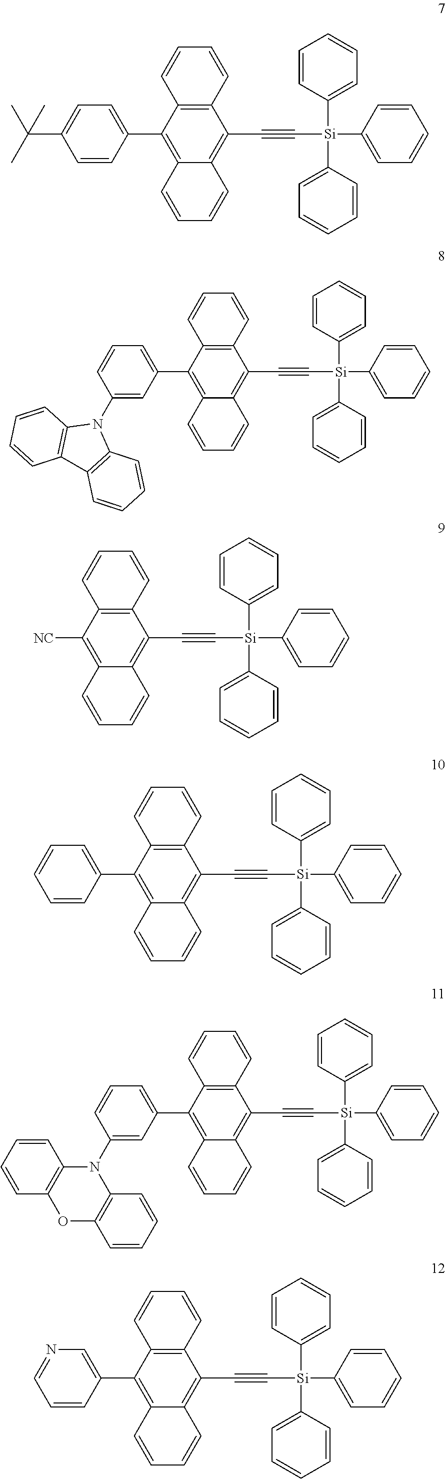

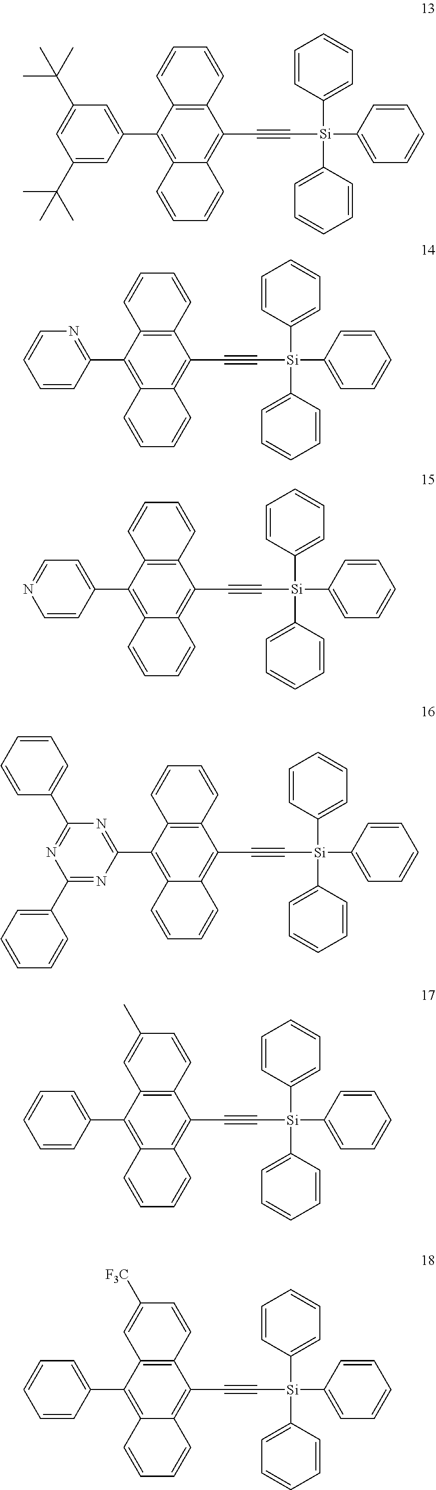

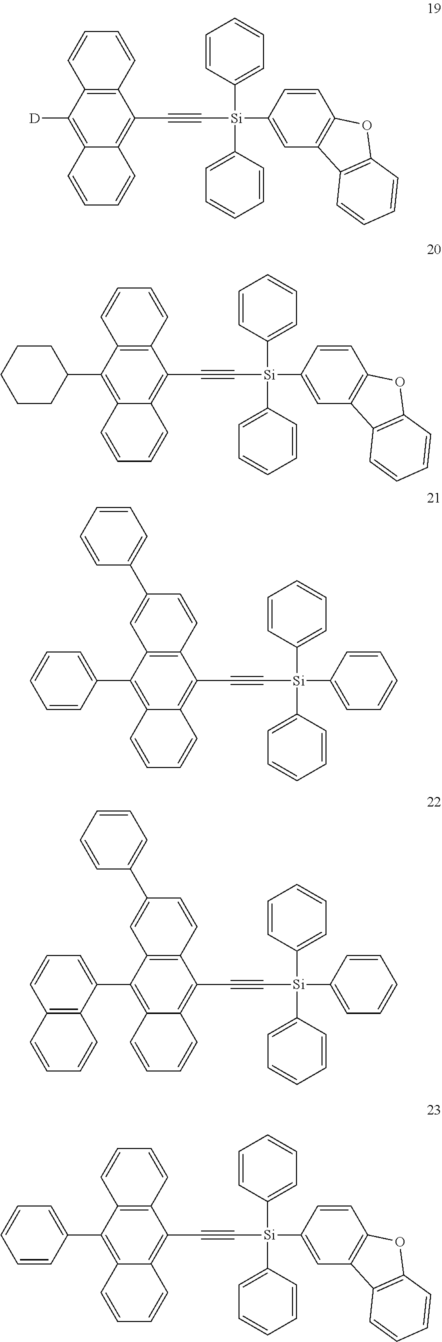 [SMILES: [2H]c1c2ccccc2c(C#C[Si](c2ccccc2)(c2ccccc2)c2ccc3oc4ccccc4c3c2)c2ccccc12 ; C(#C[Si](c1ccccc1)(c1ccccc1)c1ccc2oc3ccccc3c2c1)c1c2ccccc2c(C2CCCCC2)c2ccccc12 ; C(#C[Si](c1ccccc1)(c1ccccc1)c1ccccc1)c1c2ccccc2c(-c2ccccc2)c2cc(-c3ccccc3)ccc12 ; C(#C[Si](c1ccccc1)(c1ccccc1)c1ccccc1)c1c2ccccc2c(-c2cccc3ccccc23)c2cc(-c3ccccc3)ccc12 ; C(#C[Si](c1ccccc1)(c1ccccc1)c1ccc2oc3ccccc3c2c1)c1c2ccccc2c(-c2ccccc2)c2ccccc12]

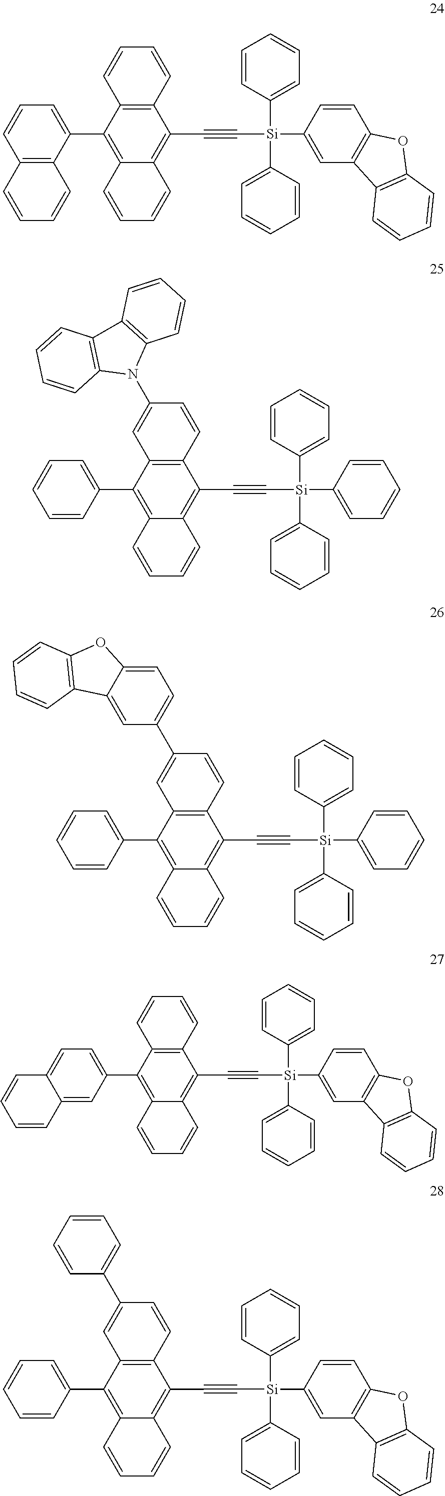 [SMILES: C(#C[Si](c1ccccc1)(c1ccccc1)c1ccc2oc3ccccc3c2c1)c1c2ccccc2c(-c2cccc3ccccc23)c2ccccc12 ; C(#C[Si](c1ccccc1)(c1ccccc1)c1ccccc1)c1c2ccccc2c(-c2ccccc2)c2cc(-n3c4ccccc4c4ccccc43)ccc12 ; C(#C[Si](c1ccccc1)(c1ccccc1)c1ccccc1)c1c2ccccc2c(-c2ccccc2)c2cc(-c3ccc4oc5ccccc5c4c3)ccc12 ; C(#C[Si](c1ccccc1)(c1ccccc1)c1ccc2oc3ccccc3c2c1)c1c2ccccc2c(-c2ccc3ccccc3c2)c2ccccc12 ; C(#C[Si](c1ccccc1)(c1ccccc1)c1ccc2oc3ccccc3c2c1)c1c2ccccc2c(-c2ccccc2)c2cc(-c3ccccc3)ccc12]

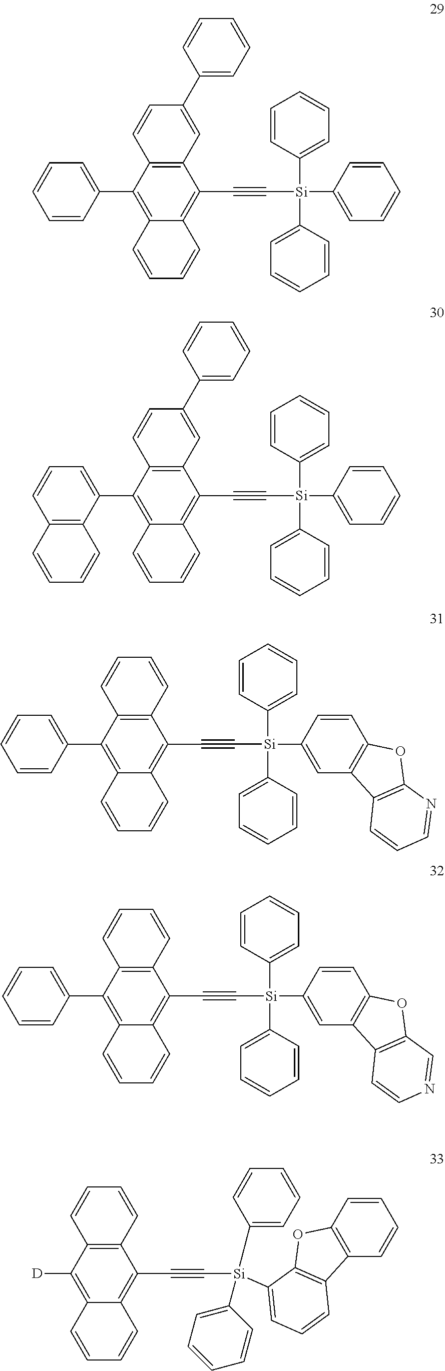 [SMILES: C(#C[Si](c1ccccc1)(c1ccccc1)c1ccccc1)c1c2ccccc2c(-c2ccccc2)c2ccc(-c3ccccc3)cc12 ; C(#C[Si](c1ccccc1)(c1ccccc1)c1ccccc1)c1c2ccccc2c(-c2cccc3ccccc23)c2ccc(-c3ccccc3)cc12 ; C(#C[Si](c1ccccc1)(c1ccccc1)c1ccc2oc3ncccc3c2c1)c1c2ccccc2c(-c2ccccc2)c2ccccc12 ; C(#C[Si](c1ccccc1)(c1ccccc1)c1ccc2oc3cnccc3c2c1)c1c2ccccc2c(-c2ccccc2)c2ccccc12 ; [2H]c1c2ccccc2c(C#C[Si](c2ccccc2)(c2ccccc2)c2cccc3c2oc2ccccc23)c2ccccc12]

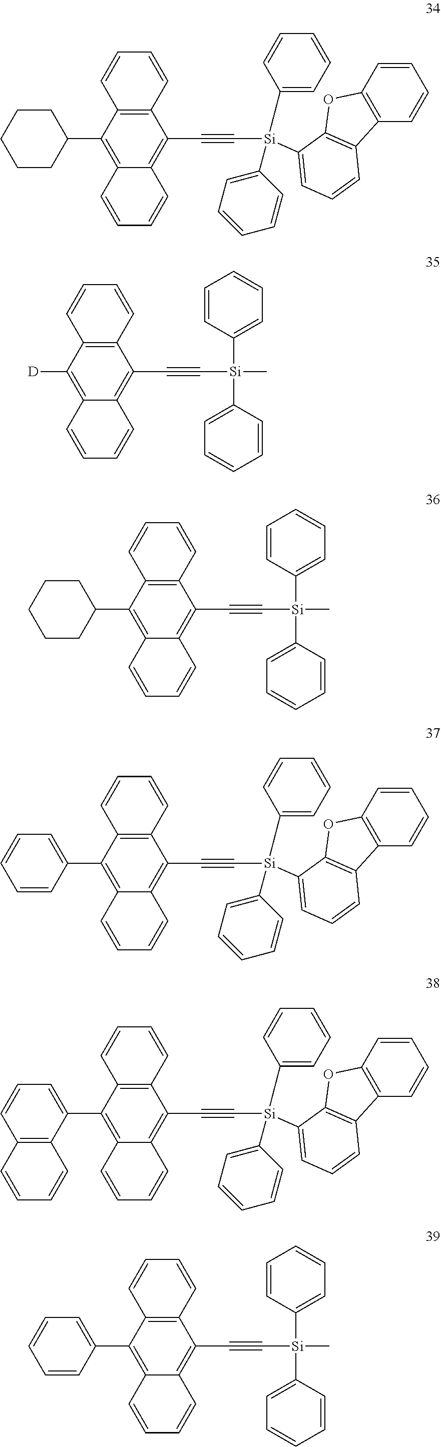

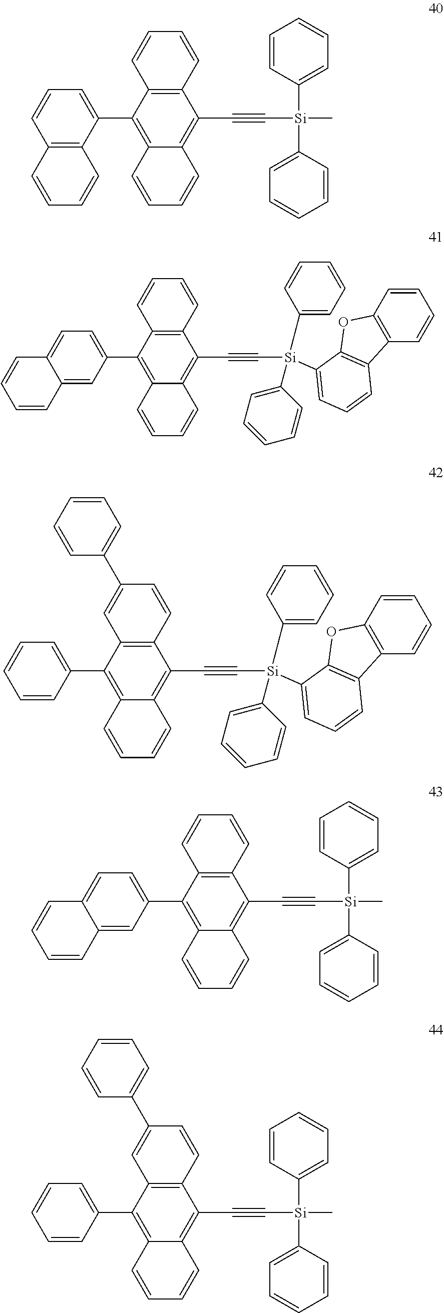

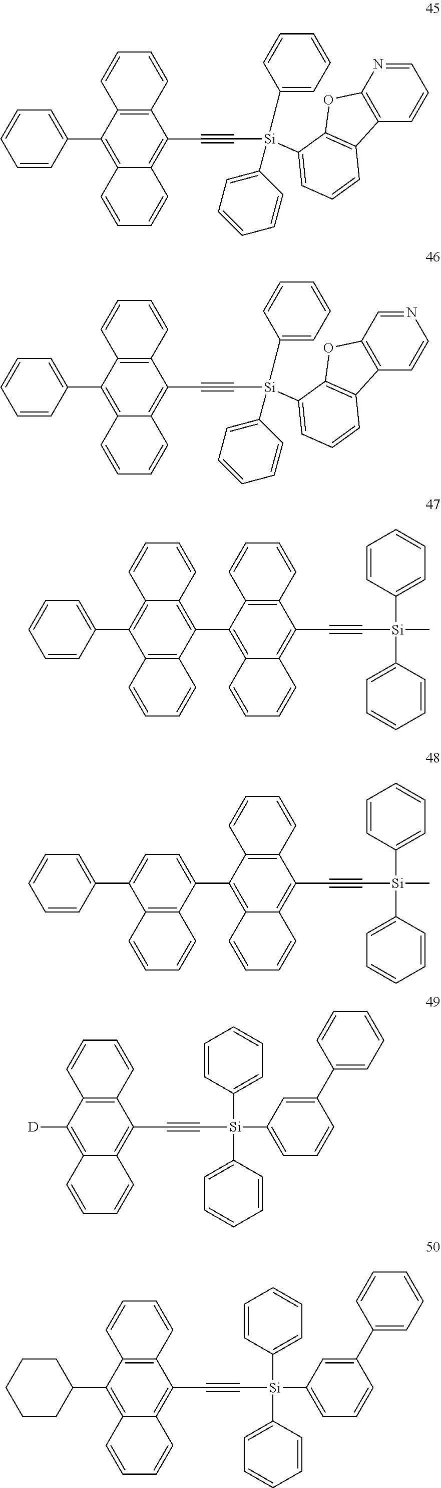

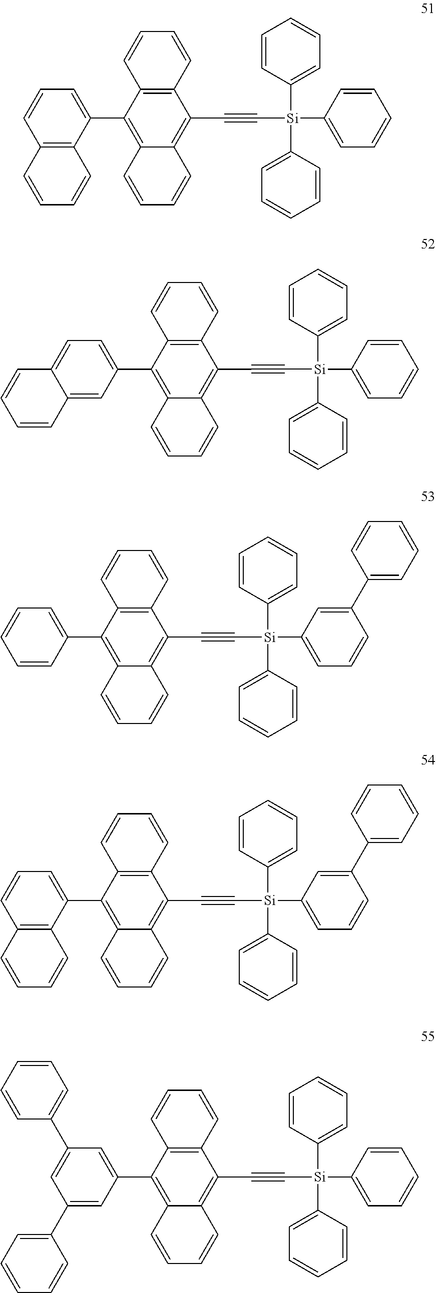 [SMILES: C(#C[Si](c1ccccc1)(c1ccccc1)c1ccccc1)c1c2ccccc2c(-c2cccc3ccccc23)c2ccccc12 ; C(#C[Si](c1ccccc1)(c1ccccc1)c1ccccc1)c1c2ccccc2c(-c2ccc3ccccc3c2)c2ccccc12 ; C(#C[Si](c1ccccc1)(c1ccccc1)c1cccc(-c2ccccc2)c1)c1c2ccccc2c(-c2ccccc2)c2ccccc12 ; C(#C[Si](c1ccccc1)(c1ccccc1)c1cccc(-c2ccccc2)c1)c1c2ccccc2c(-c2cccc3ccccc23)c2ccccc12 ; C(#C[Si](c1ccccc1)(c1ccccc1)c1ccccc1)c1c2ccccc2c(-c2cc(-c3ccccc3)cc(-c3ccccc3)c2)c2ccccc12]

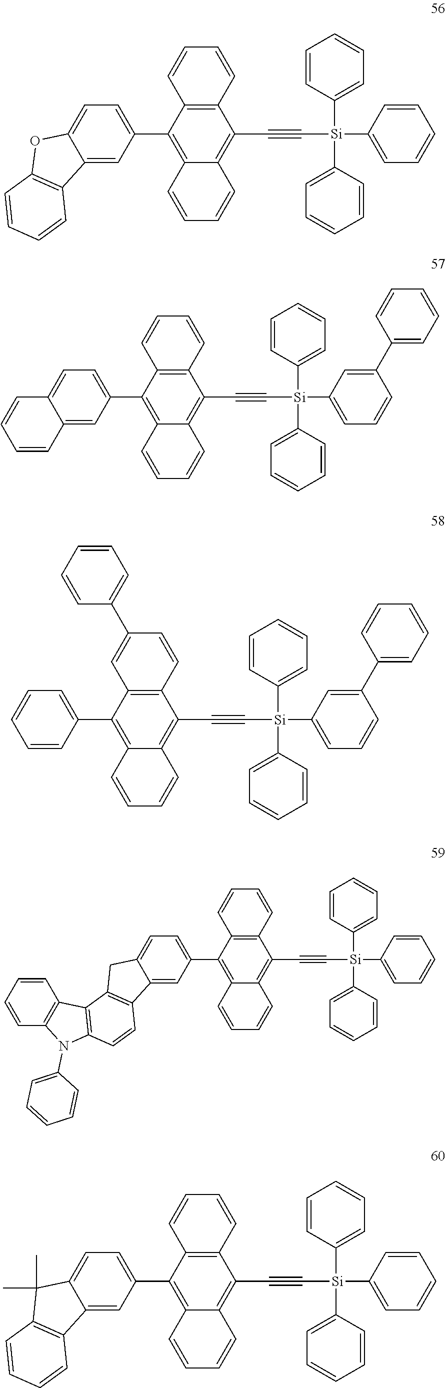 [SMILES: C(#C[Si](c1ccccc1)(c1ccccc1)c1ccccc1)c1c2ccccc2c(-c2ccc3oc4ccccc4c3c2)c2ccccc12 ; C(#C[Si](c1ccccc1)(c1ccccc1)c1cccc(-c2ccccc2)c1)c1c2ccccc2c(-c2ccc3ccccc3c2)c2ccccc12 ; C(#C[Si](c1ccccc1)(c1ccccc1)c1cccc(-c2ccccc2)c1)c1c2ccccc2c(-c2ccccc2)c2cc(-c3ccccc3)ccc12 ; C(#C[Si](c1ccccc1)(c1ccccc1)c1ccccc1)c1c2ccccc2c(-c2ccc3c(c2)-c2ccc4c(c2C3)c2ccccc2n4-c2ccccc2)c2ccccc12 ; CC1(C)c2ccccc2-c2cc(-c3c4ccccc4c(C#C[Si](c4ccccc4)(c4ccccc4)c4ccccc4)c4ccccc34)ccc21]

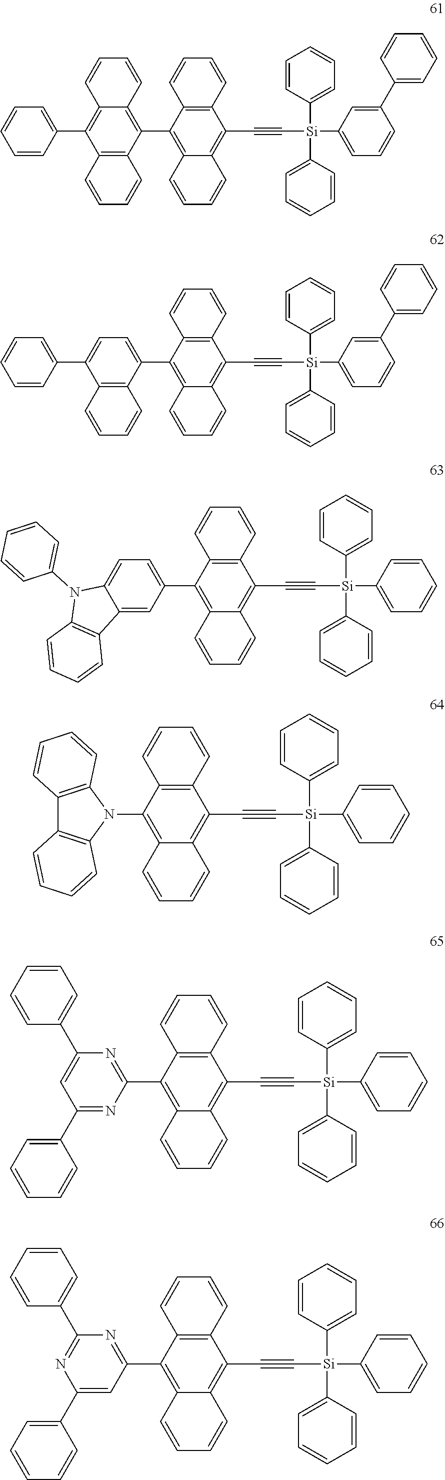

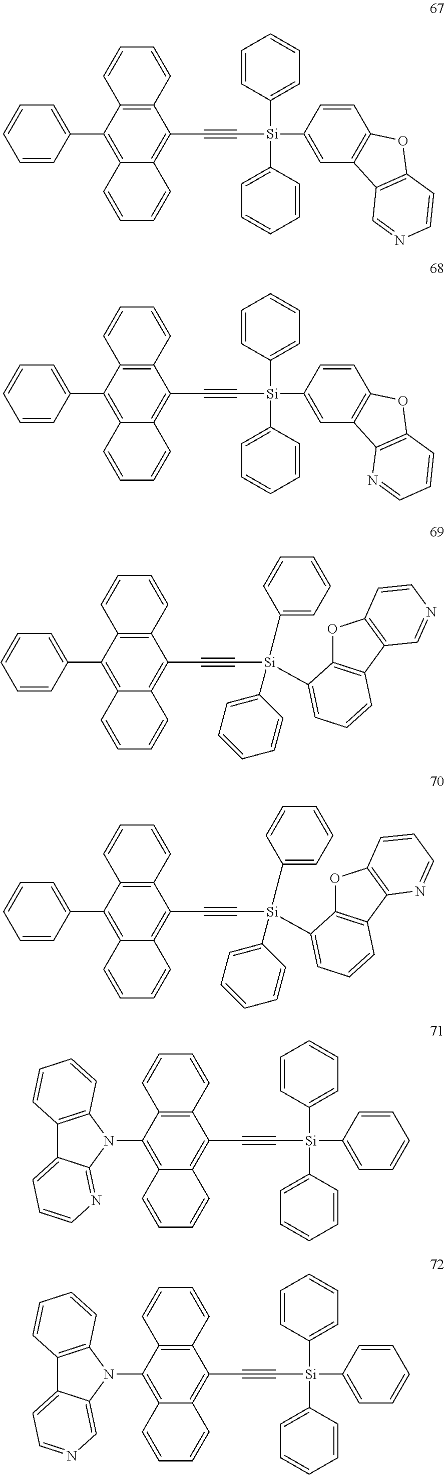

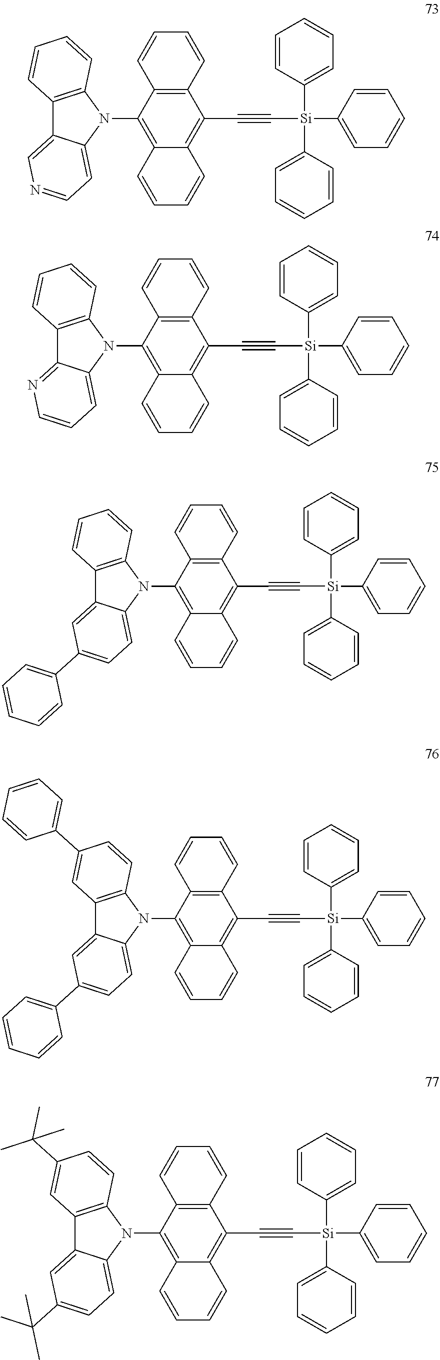

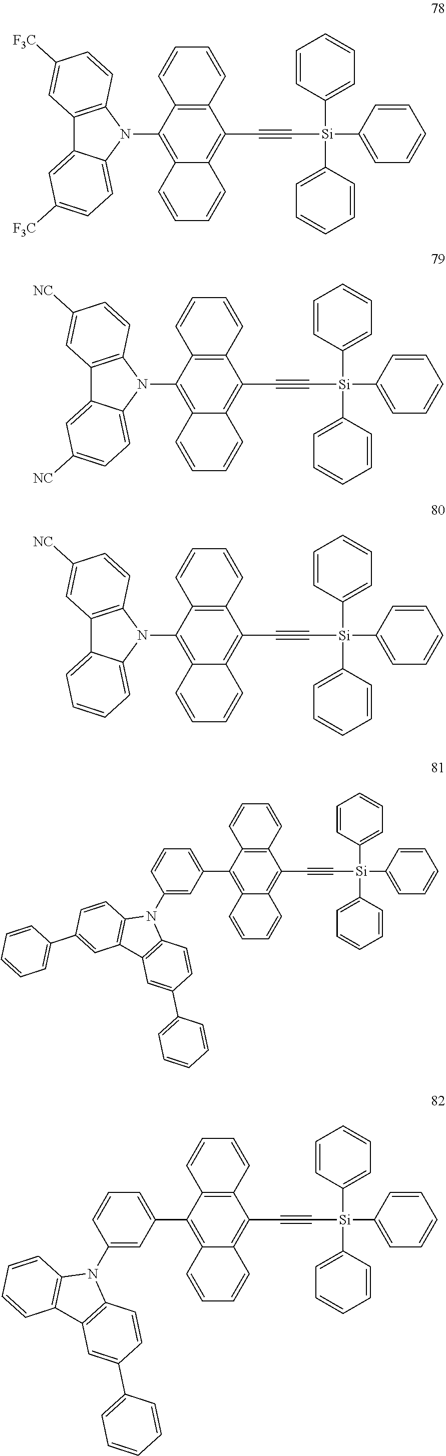 [SMILES: FC(F)(F)c1ccc2c(c1)c1cc(C(F)(F)F)ccc1n2-c1c2ccccc2c(C#C[Si](c2ccccc2)(c2ccccc2)c2ccccc2)c2ccccc12 ; N#Cc1ccc2c(c1)c1cc(C#N)ccc1n2-c1c2ccccc2c(C#C[Si](c2ccccc2)(c2ccccc2)c2ccccc2)c2ccccc12 ; N#Cc1ccc2c(c1)c1ccccc1n2-c1c2ccccc2c(C#C[Si](c2ccccc2)(c2ccccc2)c2ccccc2)c2ccccc12 ; C(#C[Si](c1ccccc1)(c1ccccc1)c1ccccc1)c1c2ccccc2c(-c2cccc(-n3c4ccc(-c5ccccc5)cc4c4cc(-c5ccccc5)ccc43)c2)c2ccccc12 ; C(#C[Si](c1ccccc1)(c1ccccc1)c1ccccc1)c1c2ccccc2c(-c2cccc(-n3c4ccccc4c4cc(-c5ccccc5)ccc43)c2)c2ccccc12]

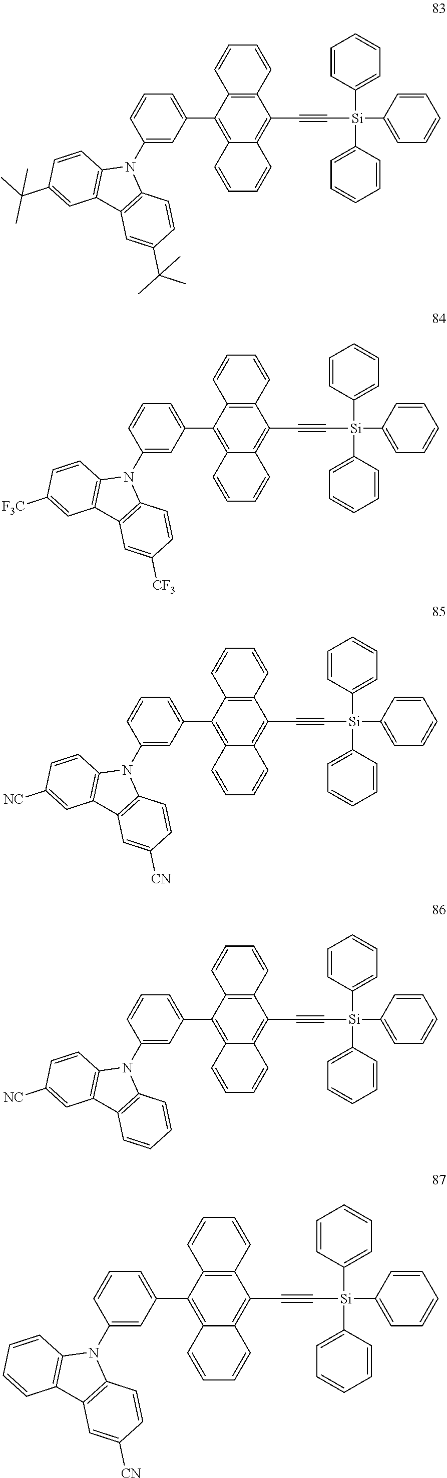

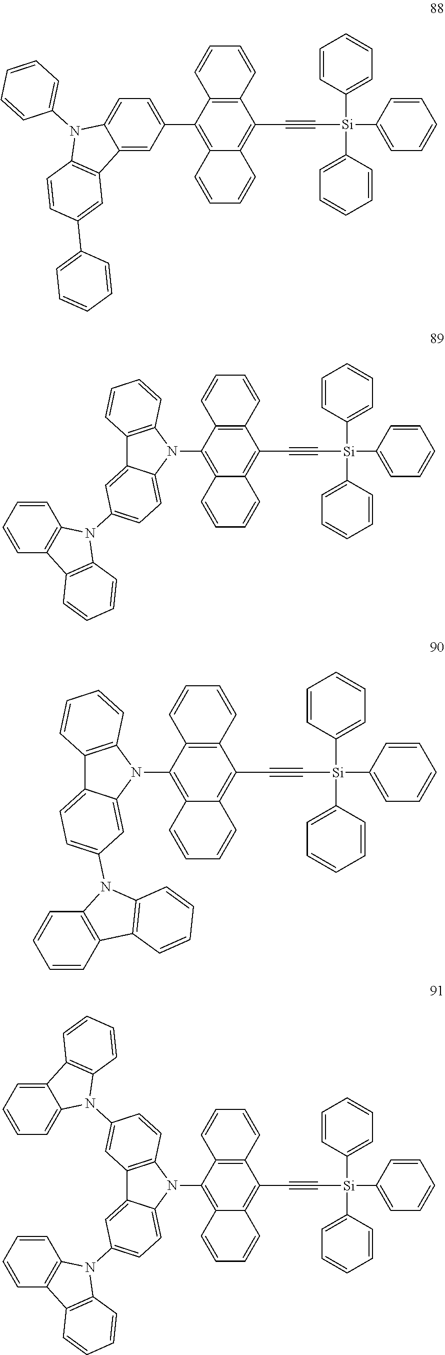 [SMILES: C(#C[Si](c1ccccc1)(c1ccccc1)c1ccccc1)c1c2ccccc2c(-c2ccc3c(c2)c2cc(-c4ccccc4)ccc2n3-c2ccccc2)c2ccccc12 ; C(#C[Si](c1ccccc1)(c1ccccc1)c1ccccc1)c1c2ccccc2c(-n2c3ccccc3c3cc(-n4c5ccccc5c5ccccc54)ccc32)c2ccccc12 ; C(#C[Si](c1ccccc1)(c1ccccc1)c1ccccc1)c1c2ccccc2c(-n2c3ccccc3c3ccc(-n4c5ccccc5c5ccccc54)cc32)c2ccccc12 ; C(#C[Si](c1ccccc1)(c1ccccc1)c1ccccc1)c1c2ccccc2c(-n2c3ccc(-n4c5ccccc5c5ccccc54)cc3c3cc(-n4c5ccccc5c5ccccc54)ccc32)c2ccccc12]

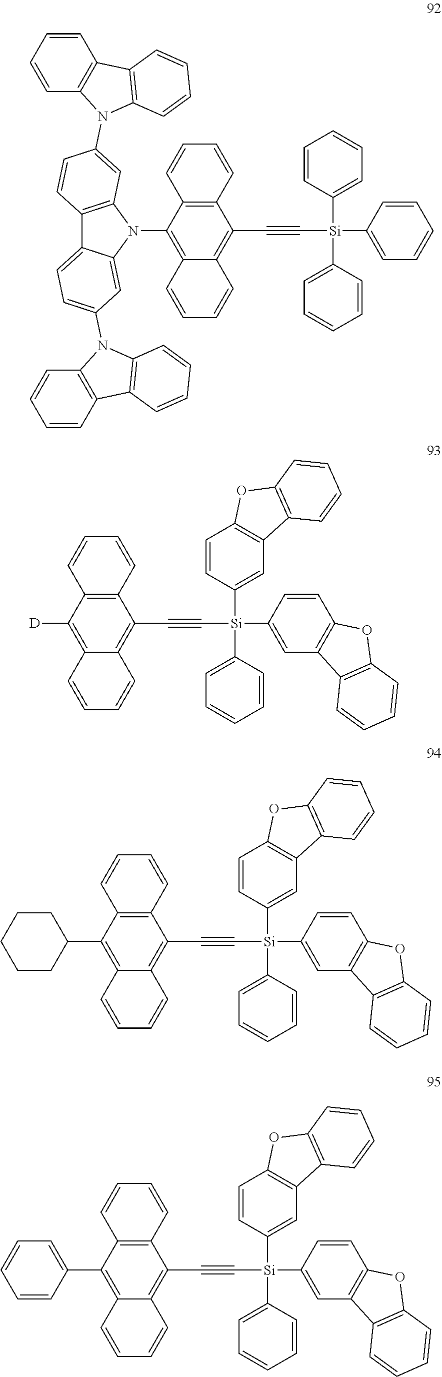 [SMILES: C(#C[Si](c1ccccc1)(c1ccccc1)c1ccccc1)c1c2ccccc2c(-n2c3cc(-n4c5ccccc5c5ccccc54)ccc3c3ccc(-n4c5ccccc5c5ccccc54)cc32)c2ccccc12 ; [2H]c1c2ccccc2c(C#C[Si](c2ccccc2)(c2ccc3oc4ccccc4c3c2)c2ccc3oc4ccccc4c3c2)c2ccccc12 ; C(#C[Si](c1ccccc1)(c1ccc2oc3ccccc3c2c1)c1ccc2oc3ccccc3c2c1)c1c2ccccc2c(C2CCCCC2)c2ccccc12 ; C(#C[Si](c1ccccc1)(c1ccc2oc3ccccc3c2c1)c1ccc2oc3ccccc3c2c1)c1c2ccccc2c(-c2ccccc2)c2ccccc12]

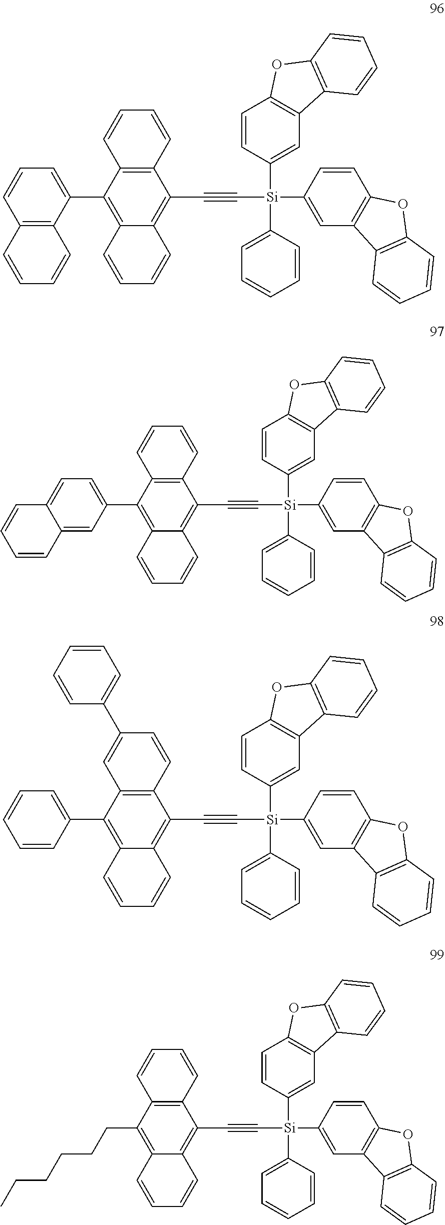 [SMILES: C(#C[Si](c1ccccc1)(c1ccc2oc3ccccc3c2c1)c1ccc2oc3ccccc3c2c1)c1c2ccccc2c(-c2cccc3ccccc23)c2ccccc12 ; C(#C[Si](c1ccccc1)(c1ccc2oc3ccccc3c2c1)c1ccc2oc3ccccc3c2c1)c1c2ccccc2c(-c2ccc3ccccc3c2)c2ccccc12 ; C(#C[Si](c1ccccc1)(c1ccc2oc3ccccc3c2c1)c1ccc2oc3ccccc3c2c1)c1c2ccccc2c(-c2ccccc2)c2cc(-c3ccccc3)ccc12 ; CCCCCCc1c2ccccc2c(C#C[Si](c2ccccc2)(c2ccc3oc4ccccc4c3c2)c2ccc3oc4ccccc4c3c2)c2ccccc12]

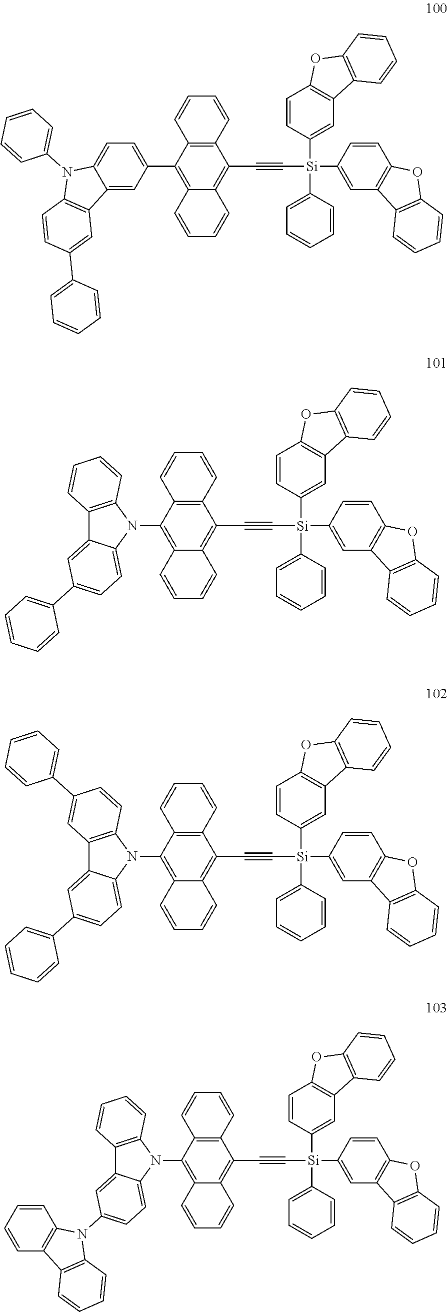 [SMILES: C(#C[Si](c1ccccc1)(c1ccc2oc3ccccc3c2c1)c1ccc2oc3ccccc3c2c1)c1c2ccccc2c(-c2ccc3c(c2)c2cc(-c4ccccc4)ccc2n3-c2ccccc2)c2ccccc12 ; C(#C[Si](c1ccccc1)(c1ccc2oc3ccccc3c2c1)c1ccc2oc3ccccc3c2c1)c1c2ccccc2c(-n2c3ccccc3c3cc(-c4ccccc4)ccc32)c2ccccc12 ; C(#C[Si](c1ccccc1)(c1ccc2oc3ccccc3c2c1)c1ccc2oc3ccccc3c2c1)c1c2ccccc2c(-n2c3ccc(-c4ccccc4)cc3c3cc(-c4ccccc4)ccc32)c2ccccc12 ; C(#C[Si](c1ccccc1)(c1ccc2oc3ccccc3c2c1)c1ccc2oc3ccccc3c2c1)c1c2ccccc2c(-n2c3ccccc3c3cc(-n4c5ccccc5c5ccccc54)ccc32)c2ccccc12]

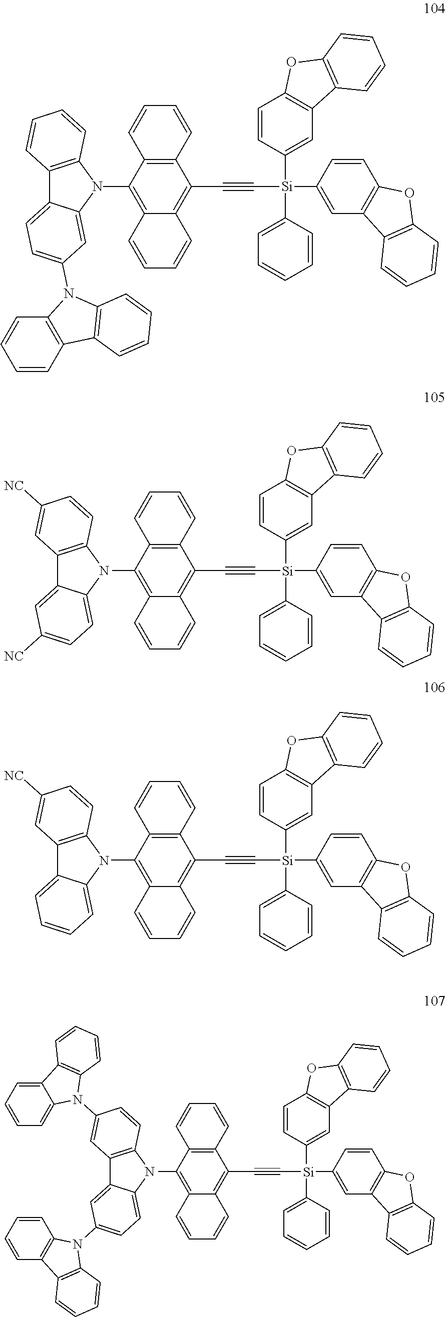 [SMILES: C(#C[Si](c1ccccc1)(c1ccc2oc3ccccc3c2c1)c1ccc2oc3ccccc3c2c1)c1c2ccccc2c(-n2c3ccccc3c3ccc(-n4c5ccccc5c5ccccc54)cc32)c2ccccc12 ; N#Cc1ccc2c(c1)c1cc(C#N)ccc1n2-c1c2ccccc2c(C#C[Si](c2ccccc2)(c2ccc3oc4ccccc4c3c2)c2ccc3oc4ccccc4c3c2)c2ccccc12 ; N#Cc1ccc2c(c1)c1ccccc1n2-c1c2ccccc2c(C#C[Si](c2ccccc2)(c2ccc3oc4ccccc4c3c2)c2ccc3oc4ccccc4c3c2)c2ccccc12 ; C(#C[Si](c1ccccc1)(c1ccc2oc3ccccc3c2c1)c1ccc2oc3ccccc3c2c1)c1c2ccccc2c(-n2c3ccc(-n4c5ccccc5c5ccccc54)cc3c3cc(-n4c5ccccc5c5ccccc54)ccc32)c2ccccc12]

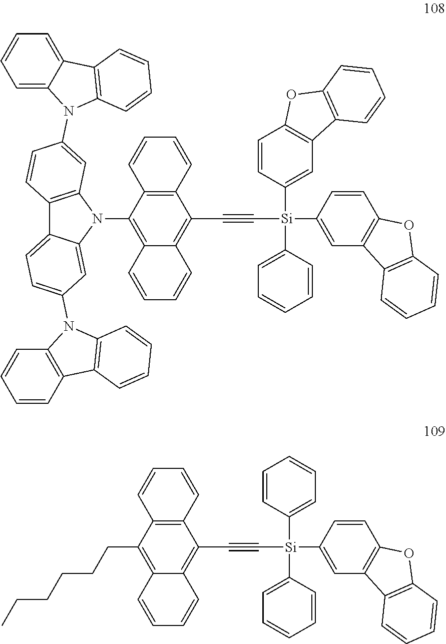 [SMILES: C(#C[Si](c1ccccc1)(c1ccc2oc3ccccc3c2c1)c1ccc2oc3ccccc3c2c1)c1c2ccccc2c(-n2c3cc(-n4c5ccccc5c5ccccc54)ccc3c3ccc(-n4c5ccccc5c5ccccc54)cc32)c2ccccc12 ; CCCCCCc1c2ccccc2c(C#C[Si](c2ccccc2)(c2ccccc2)c2ccc3oc4ccccc4c3c2)c2ccccc12]

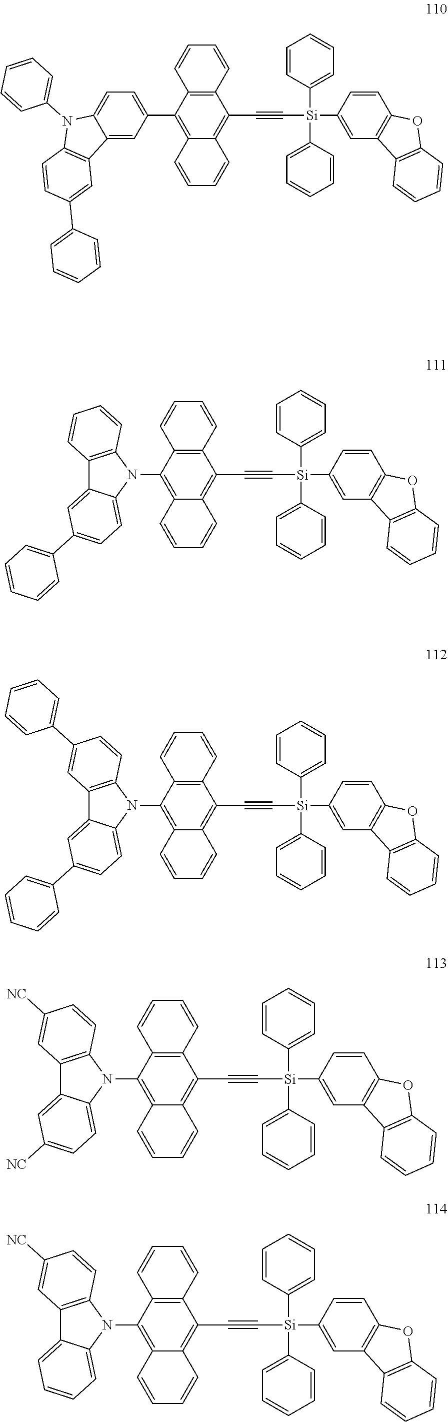

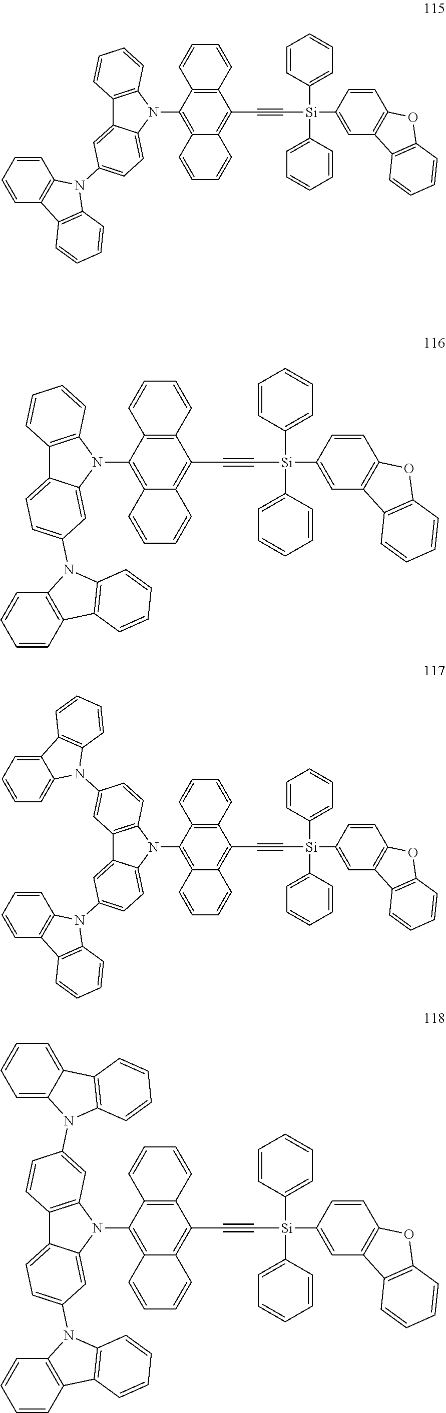

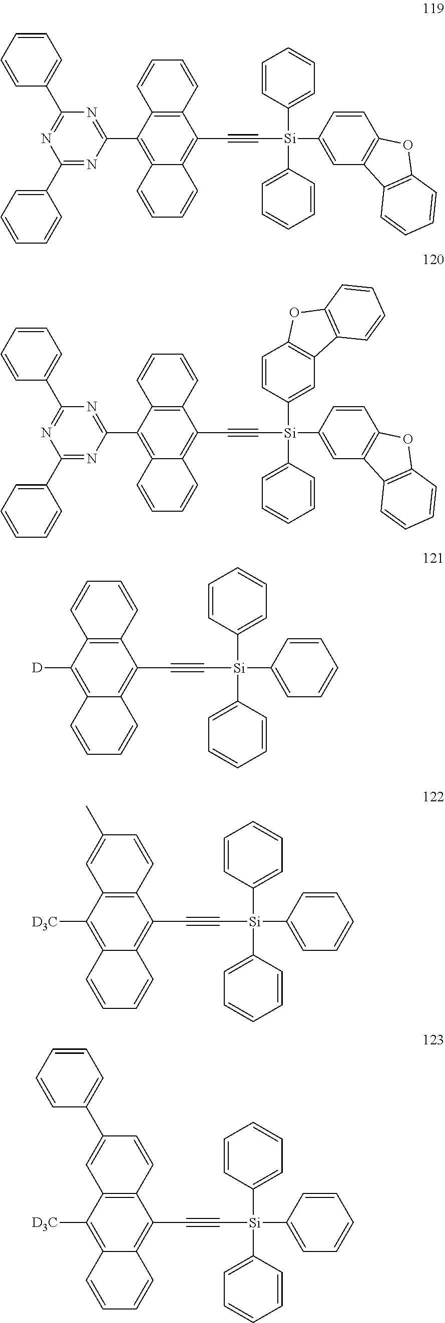 [SMILES: C(#C[Si](c1ccccc1)(c1ccccc1)c1ccc2oc3ccccc3c2c1)c1c2ccccc2c(-c2nc(-c3ccccc3)nc(-c3ccccc3)n2)c2ccccc12 ; C(#C[Si](c1ccccc1)(c1ccc2oc3ccccc3c2c1)c1ccc2oc3ccccc3c2c1)c1c2ccccc2c(-c2nc(-c3ccccc3)nc(-c3ccccc3)n2)c2ccccc12 ; [2H]c1c2ccccc2c(C#C[Si](c2ccccc2)(c2ccccc2)c2ccccc2)c2ccccc12 ; [2H]C([2H])([2H])c1c2ccccc2c(C#C[Si](c2ccccc2)(c2ccccc2)c2ccccc2)c2ccc(C)cc12 ; [2H]C([2H])([2H])c1c2ccccc2c(C#C[Si](c2ccccc2)(c2ccccc2)c2ccccc2)c2ccc(-c3ccccc3)cc12]

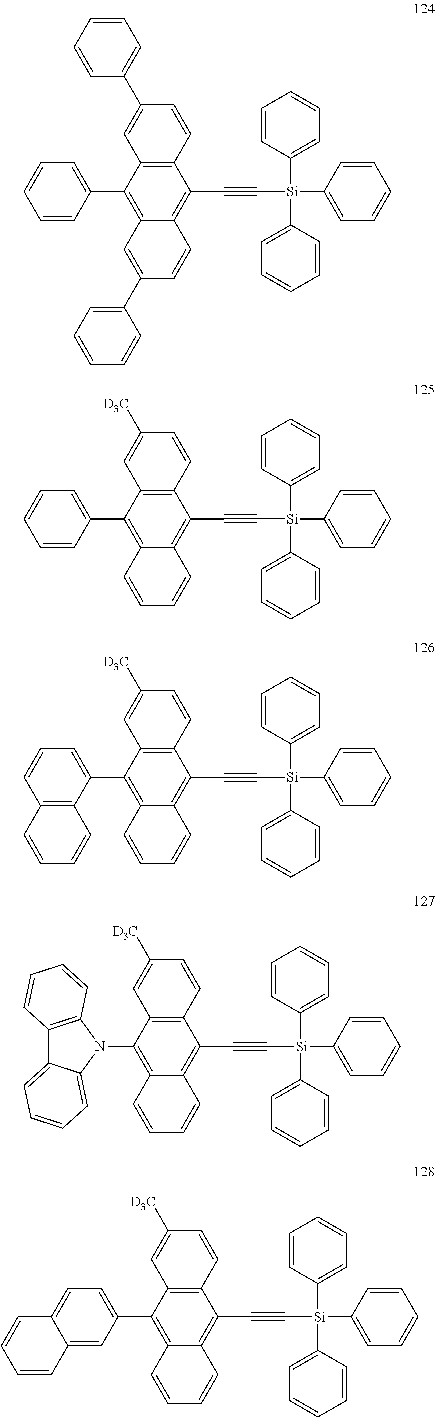

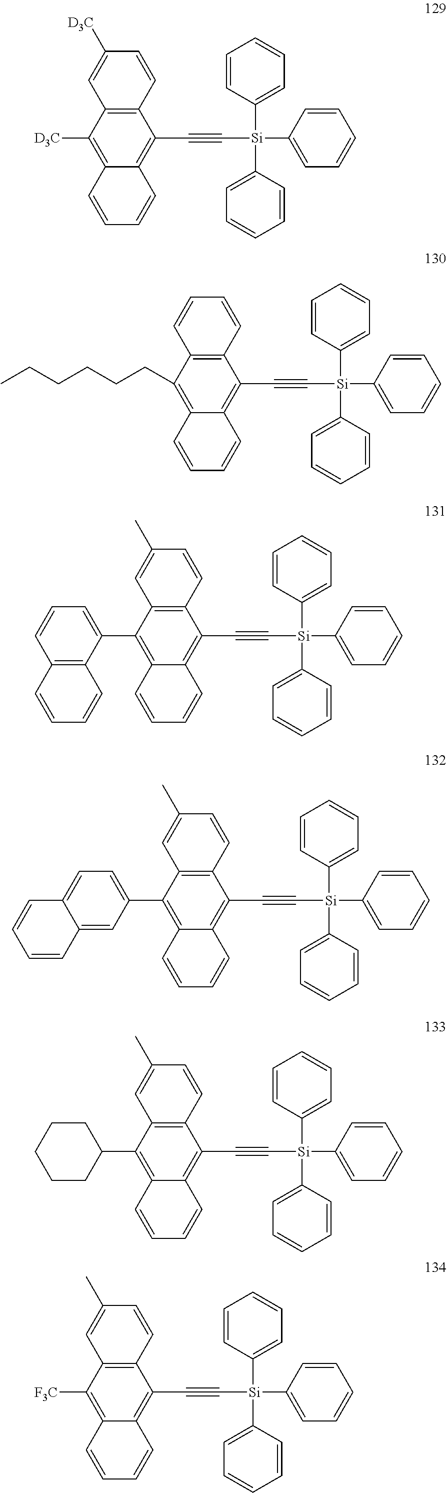 [SMILES: [2H]C([2H])([2H])c1ccc2c(C#C[Si](c3ccccc3)(c3ccccc3)c3ccccc3)c3ccccc3c(C([2H])([2H])[2H])c2c1 ; CCCCCCc1c2ccccc2c(C#C[Si](c2ccccc2)(c2ccccc2)c2ccccc2)c2ccccc12 ; Cc1ccc2c(C#C[Si](c3ccccc3)(c3ccccc3)c3ccccc3)c3ccccc3c(-c3cccc4ccccc34)c2c1 ; Cc1ccc2c(C#C[Si](c3ccccc3)(c3ccccc3)c3ccccc3)c3ccccc3c(-c3ccc4ccccc4c3)c2c1 ; Cc1ccc2c(C#C[Si](c3ccccc3)(c3ccccc3)c3ccccc3)c3ccccc3c(C3CCCCC3)c2c1 ; Cc1ccc2c(C#C[Si](c3ccccc3)(c3ccccc3)c3ccccc3)c3ccccc3c(C(F)(F)F)c2c1]

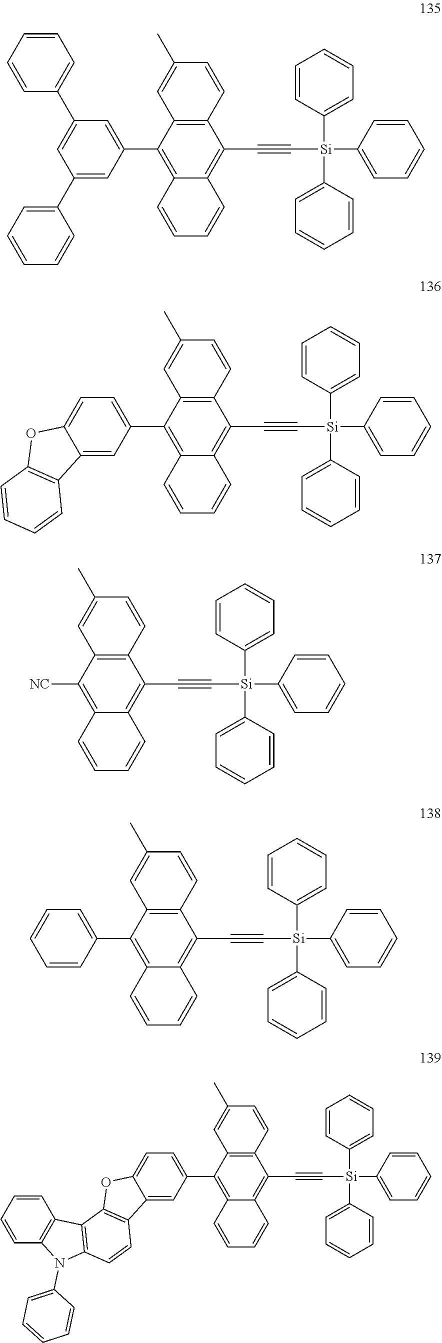 [SMILES: Cc1ccc2c(C#C[Si](c3ccccc3)(c3ccccc3)c3ccccc3)c3ccccc3c(-c3cc(-c4ccccc4)cc(-c4ccccc4)c3)c2c1 ; Cc1ccc2c(C#C[Si](c3ccccc3)(c3ccccc3)c3ccccc3)c3ccccc3c(-c3ccc4oc5ccccc5c4c3)c2c1 ; Cc1ccc2c(C#C[Si](c3ccccc3)(c3ccccc3)c3ccccc3)c3ccccc3c(C#N)c2c1 ; Cc1ccc2c(C#C[Si](c3ccccc3)(c3ccccc3)c3ccccc3)c3ccccc3c(-c3ccccc3)c2c1 ; Cc1ccc2c(C#C[Si](c3ccccc3)(c3ccccc3)c3ccccc3)c3ccccc3c(-c3ccc4oc5c(ccc6c5c5ccccc5n6-c5ccccc5)c4c3)c2c1]

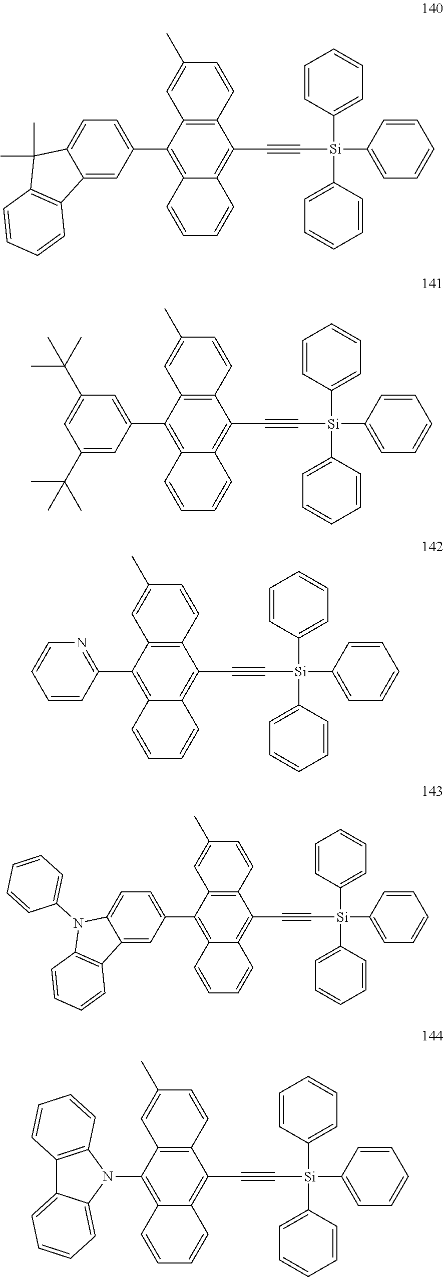 [SMILES: Cc1ccc2c(C#C[Si](c3ccccc3)(c3ccccc3)c3ccccc3)c3ccccc3c(-c3ccc4c(c3)-c3ccccc3C4(C)C)c2c1 ; Cc1ccc2c(C#C[Si](c3ccccc3)(c3ccccc3)c3ccccc3)c3ccccc3c(-c3cc(C(C)(C)C)cc(C(C)(C)C)c3)c2c1 ; Cc1ccc2c(C#C[Si](c3ccccc3)(c3ccccc3)c3ccccc3)c3ccccc3c(-c3ccccn3)c2c1 ; Cc1ccc2c(C#C[Si](c3ccccc3)(c3ccccc3)c3ccccc3)c3ccccc3c(-c3ccc4c(c3)c3ccccc3n4-c3ccccc3)c2c1 ; Cc1ccc2c(C#C[Si](c3ccccc3)(c3ccccc3)c3ccccc3)c3ccccc3c(-n3c4ccccc4c4ccccc43)c2c1]

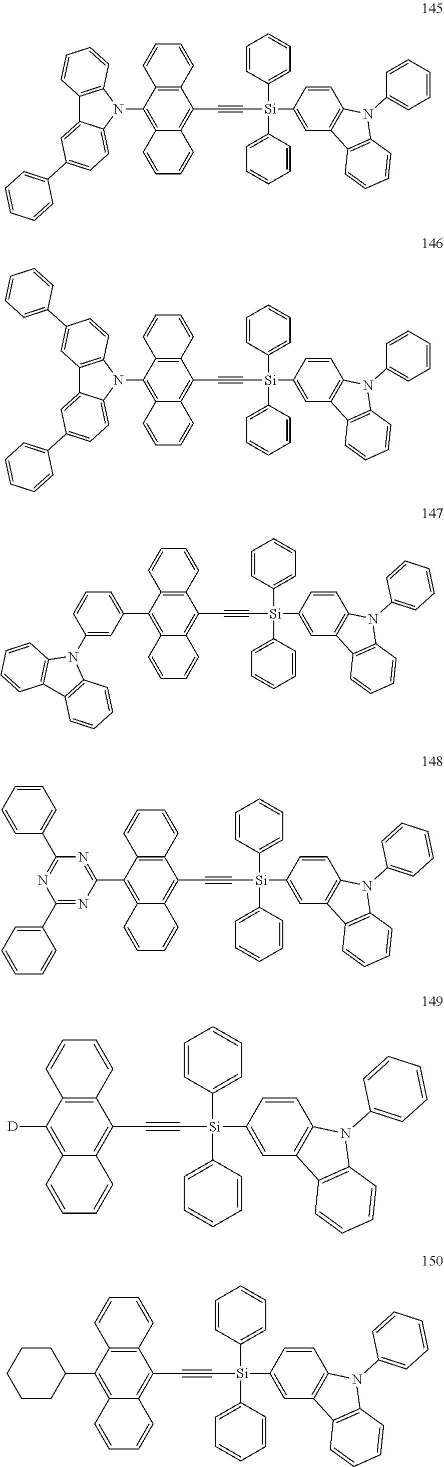

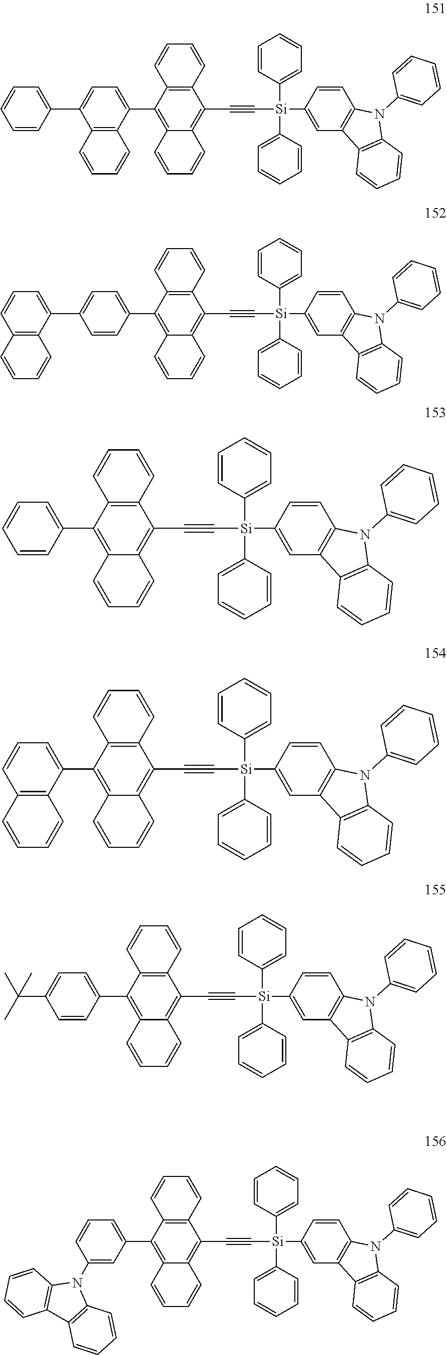

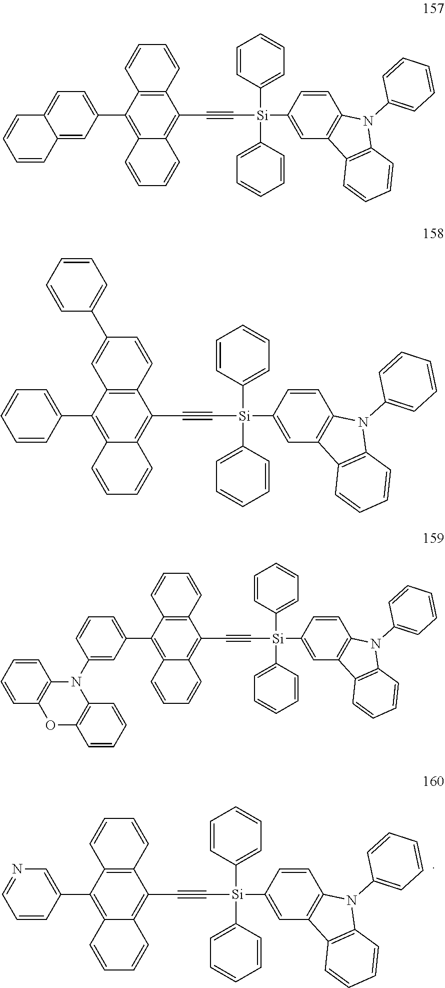 [SMILES: C(#C[Si](c1ccccc1)(c1ccccc1)c1ccc2c(c1)c1ccccc1n2-c1ccccc1)c1c2ccccc2c(-c2ccc3ccccc3c2)c2ccccc12 ; C(#C[Si](c1ccccc1)(c1ccccc1)c1ccc2c(c1)c1ccccc1n2-c1ccccc1)c1c2ccccc2c(-c2ccccc2)c2cc(-c3ccccc3)ccc12 ; C(#C[Si](c1ccccc1)(c1ccccc1)c1ccc2c(c1)c1ccccc1n2-c1ccccc1)c1c2ccccc2c(-c2cccc(N3c4ccccc4Oc4ccccc43)c2)c2ccccc12 ; C(#C[Si](c1ccccc1)(c1ccccc1)c1ccc2c(c1)c1ccccc1n2-c1ccccc1)c1c2ccccc2c(-c2cccnc2)c2ccccc12]

In an embodiment, the fluorescent host may be represented by Formula FH-2:

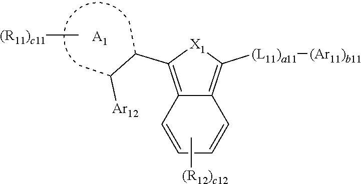

wherein, in Formula FH-2,

X₁ may be O or S,

A₁ may be a C₅-C₆₀ carbocyclic group or a C₁-C₆₀ heterocyclic group,

L₁₁ may be a substituted or unsubstituted C₅-C₆₀ carbocyclic group or a substituted or unsubstituted C₁-C₆₀ heterocyclic group,

a11 may be an integer from 0 to 3,

Ar₁₁ and Ar₁₂ may each independently be a C₆-C₆₀ aryl group, a C₁-C₆₀ heteroaryl group, a monovalent non-aromatic condensed polycyclic group, or a monovalent non-aromatic condensed heteropolycyclic group, each unsubstituted or substituted with at least one Rₐ,

b11 may be an integer from 1 to 5,

R₁₁, R₁₂, and Ra may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇),

c11 may be an integer from 1 to 20,

c12 may be an integer of 1 to 4,

when c11 is 2 or more, adjacent two of R₁₁(s) may optionally be bonded to each other to form a substituted or unsubstituted C₅-C₃₀ carbocyclic group or a substituted or unsubstituted C₁-C₃₀ heterocyclic group,

when c12 is 2 or more, adjacent two of R₁₂(s) may optionally be bonded to each other to form a substituted or unsubstituted C₅-C₃₀ carbocyclic group or a substituted or unsubstituted C₁-C₃₀ heterocyclic group,

A₁ and Ar₁₂ may optionally be bonded to each other via a single bond or a first linking group including *—Ar₃₁—*′, *—O—*′, *—S—*′, *—[C(R₃₁)(R₃₂)]ₖ₁₁—*′, *—C(R₃₁)═*′, *═C(R₃₁)—*′, *—C(R₃₁)═C(R₃₂)—*′, *—C(═O)—*′, *—C(═S)—*′, *—C≡C—*′, *—N(R₃₁)—*′, *—P(R₃₁)—*′, *—[Si(R₃₁)(R₃₂)]ₖ₁₁—*′, or *—P(R₃₁)(R₃₂)—*′ to form a substituted or unsubstituted C₅-C₃₀ carbocyclic group or a substituted or unsubstituted C₁-C₃₀ heterocyclic group,

Ar₃₁ is a C₅-C₃₀ carbocyclic group,

R₃₁ and R₃₂ are each the same as described in connection with R₁₁, and

k11 is 1, 2, 3, and 4.

In an embodiment, the fluorescent host represented by Formula FH-2 may be Group FH2, but embodiments of the present disclosure are not limited thereto:

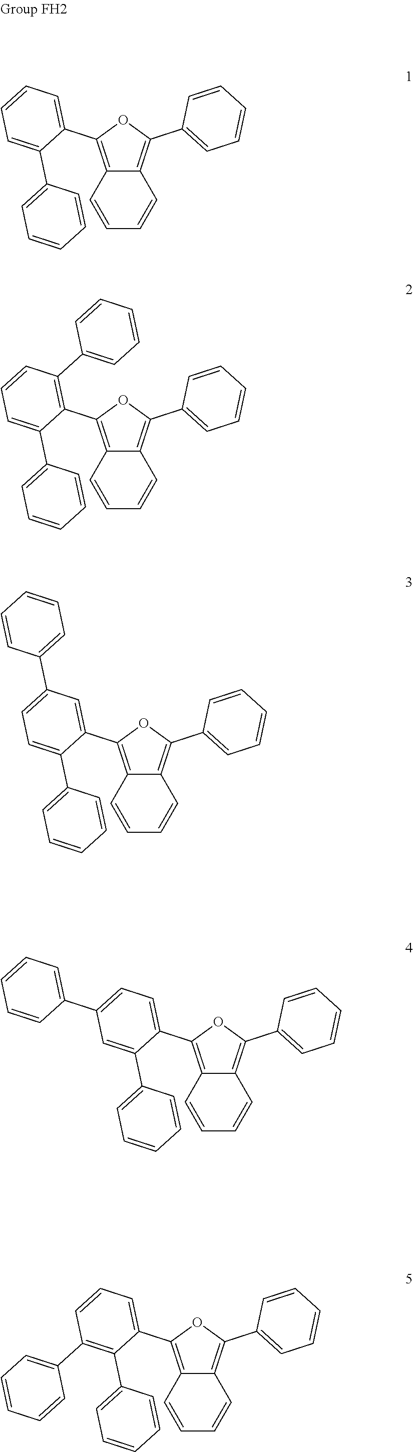 [SMILES: c1ccc(-c2ccccc2-c2oc(-c3ccccc3)c3ccccc23)cc1 ; c1ccc(-c2cccc(-c3ccccc3)c2-c2oc(-c3ccccc3)c3ccccc23)cc1 ; c1ccc(-c2ccc(-c3ccccc3)c(-c3oc(-c4ccccc4)c4ccccc34)c2)cc1 ; c1ccc(-c2ccc(-c3oc(-c4ccccc4)c4ccccc34)c(-c3ccccc3)c2)cc1 ; c1ccc(-c2cccc(-c3oc(-c4ccccc4)c4ccccc34)c2-c2ccccc2)cc1]

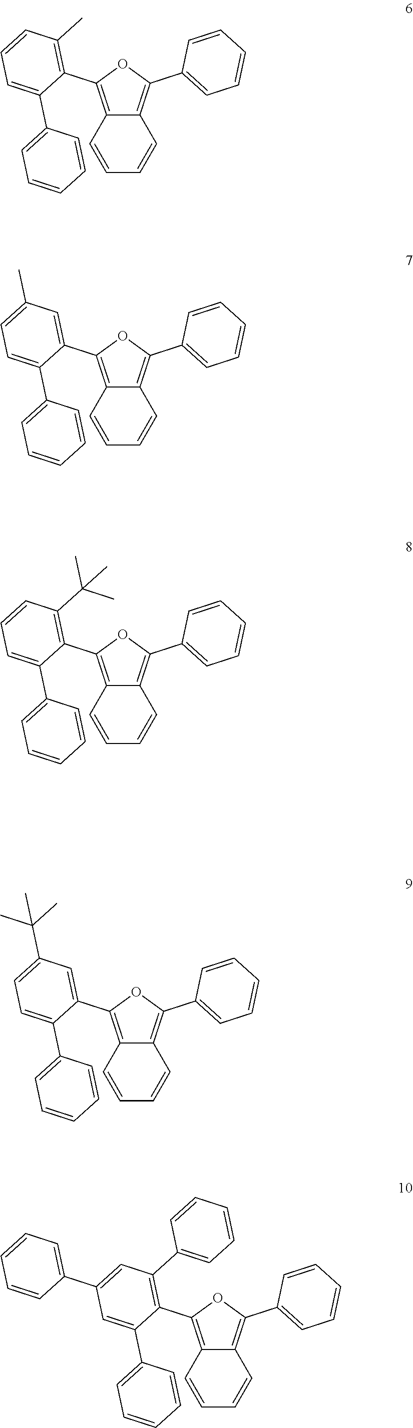 [SMILES: Cc1cccc(-c2ccccc2)c1-c1oc(-c2ccccc2)c2ccccc12 ; Cc1ccc(-c2ccccc2)c(-c2oc(-c3ccccc3)c3ccccc23)c1 ; CC(C)(C)c1cccc(-c2ccccc2)c1-c1oc(-c2ccccc2)c2ccccc12 ; CC(C)(C)c1ccc(-c2ccccc2)c(-c2oc(-c3ccccc3)c3ccccc23)c1 ; c1ccc(-c2cc(-c3ccccc3)c(-c3oc(-c4ccccc4)c4ccccc34)c(-c3ccccc3)c2)cc1]

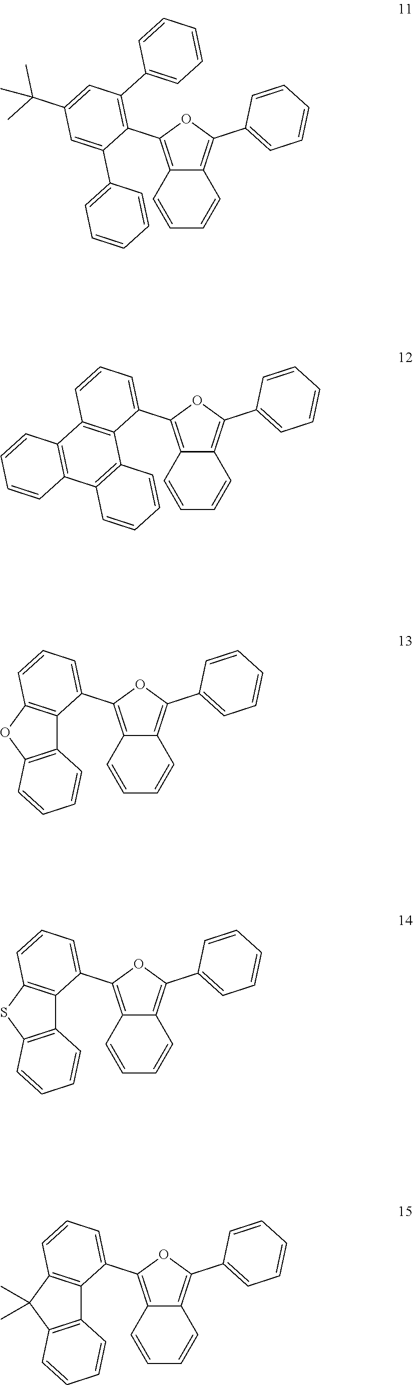 [SMILES: CC(C)(C)c1cc(-c2ccccc2)c(-c2oc(-c3ccccc3)c3ccccc23)c(-c2ccccc2)c1 ; c1ccc(-c2oc(-c3cccc4c5ccccc5c5ccccc5c34)c3ccccc23)cc1 ; c1ccc(-c2oc(-c3cccc4oc5ccccc5c34)c3ccccc23)cc1 ; c1ccc(-c2oc(-c3cccc4sc5ccccc5c34)c3ccccc23)cc1 ; CC1(C)c2ccccc2-c2c(-c3oc(-c4ccccc4)c4ccccc34)cccc21]

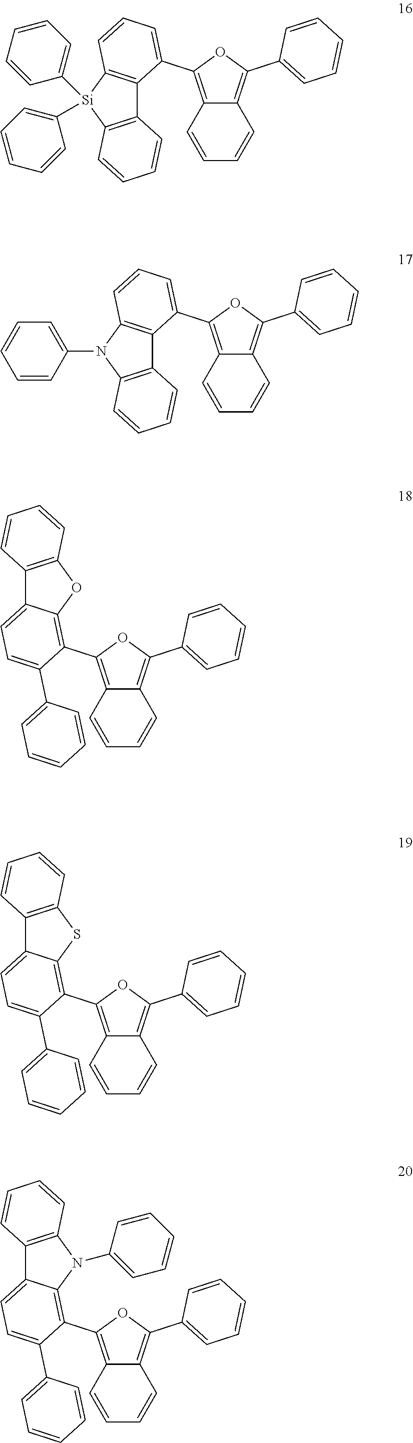 [SMILES: c1ccc(-c2oc(-c3cccc4c3-c3ccccc3[Si]4(c3ccccc3)c3ccccc3)c3ccccc23)cc1 ; c1ccc(-c2oc(-c3cccc4c3c3ccccc3n4-c3ccccc3)c3ccccc23)cc1 ; c1ccc(-c2ccc3c(oc4ccccc43)c2-c2oc(-c3ccccc3)c3ccccc23)cc1 ; c1ccc(-c2ccc3c(sc4ccccc43)c2-c2oc(-c3ccccc3)c3ccccc23)cc1 ; c1ccc(-c2ccc3c4ccccc4n(-c4ccccc4)c3c2-c2oc(-c3ccccc3)c3ccccc23)cc1]

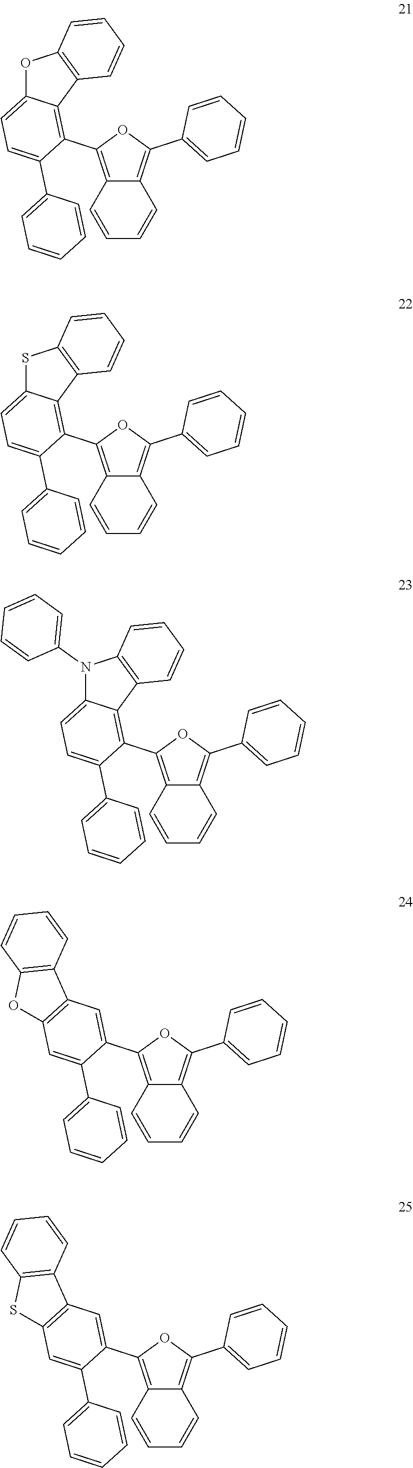 [SMILES: c1ccc(-c2ccc3oc4ccccc4c3c2-c2oc(-c3ccccc3)c3ccccc23)cc1 ; c1ccc(-c2ccc3sc4ccccc4c3c2-c2oc(-c3ccccc3)c3ccccc23)cc1 ; c1ccc(-c2ccc3c(c2-c2oc(-c4ccccc4)c4ccccc24)c2ccccc2n3-c2ccccc2)cc1 ; c1ccc(-c2cc3oc4ccccc4c3cc2-c2oc(-c3ccccc3)c3ccccc23)cc1 ; c1ccc(-c2cc3sc4ccccc4c3cc2-c2oc(-c3ccccc3)c3ccccc23)cc1]

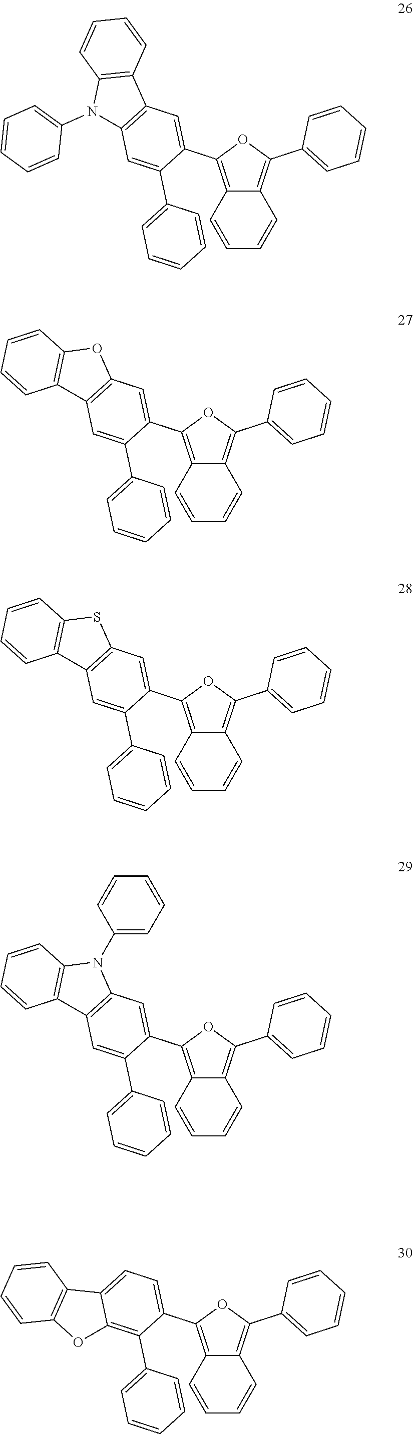 [SMILES: c1ccc(-c2cc3c(cc2-c2oc(-c4ccccc4)c4ccccc24)c2ccccc2n3-c2ccccc2)cc1 ; c1ccc(-c2cc3c(cc2-c2oc(-c4ccccc4)c4ccccc24)oc2ccccc23)cc1 ; c1ccc(-c2cc3c(cc2-c2oc(-c4ccccc4)c4ccccc24)sc2ccccc23)cc1 ; c1ccc(-c2cc3c4ccccc4n(-c4ccccc4)c3cc2-c2oc(-c3ccccc3)c3ccccc23)cc1 ; c1ccc(-c2oc(-c3ccc4c(oc5ccccc54)c3-c3ccccc3)c3ccccc23)cc1]

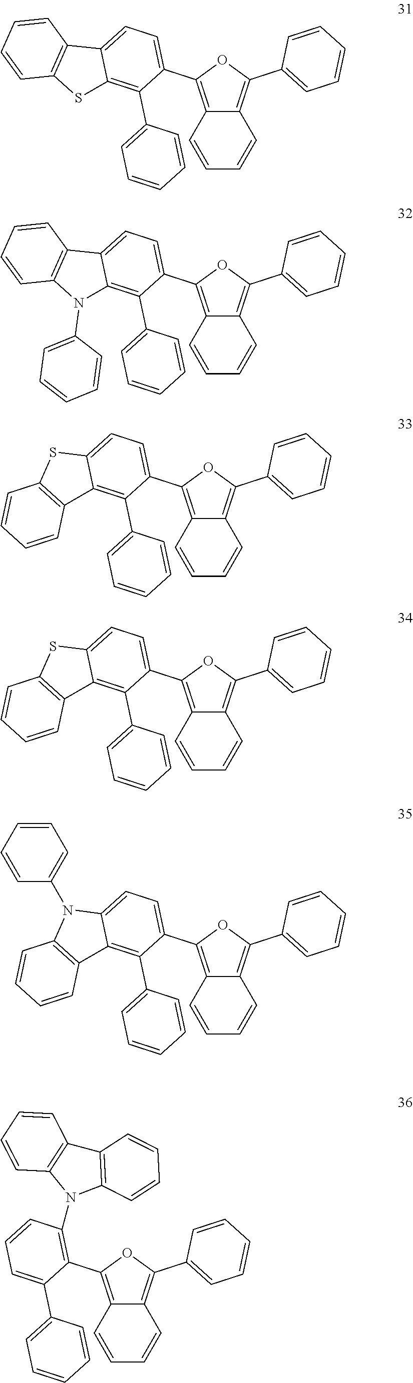

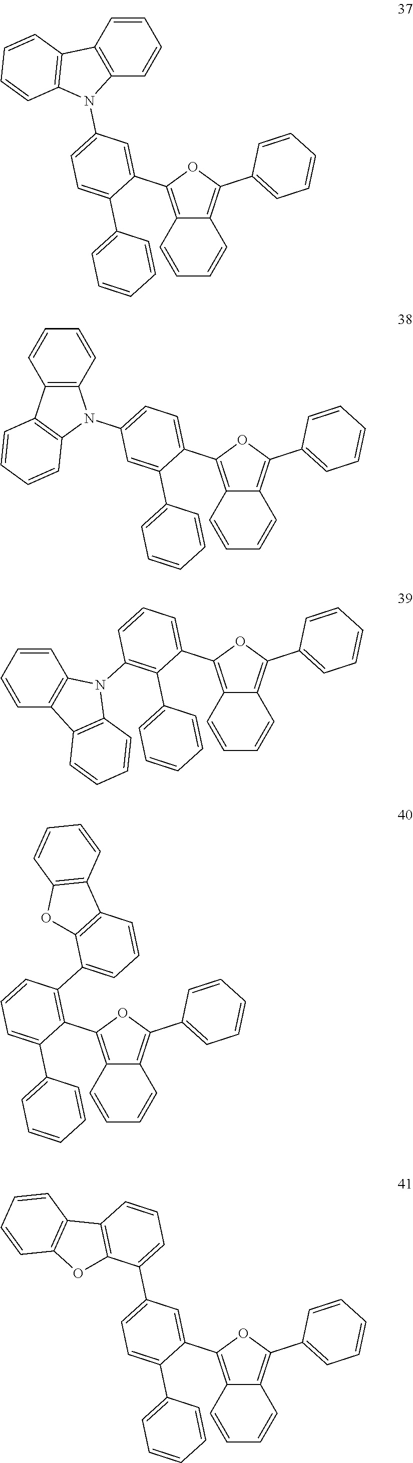

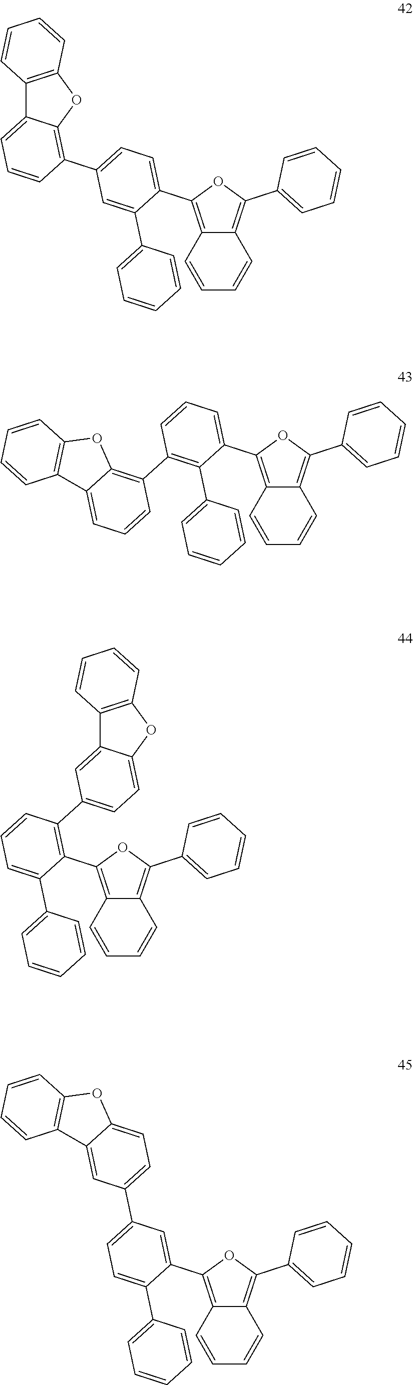 [SMILES: c1ccc(-c2cc(-c3cccc4c3oc3ccccc34)ccc2-c2oc(-c3ccccc3)c3ccccc23)cc1 ; c1ccc(-c2c(-c3oc(-c4ccccc4)c4ccccc34)cccc2-c2cccc3c2oc2ccccc23)cc1 ; c1ccc(-c2cccc(-c3ccc4oc5ccccc5c4c3)c2-c2oc(-c3ccccc3)c3ccccc23)cc1 ; c1ccc(-c2ccc(-c3ccc4oc5ccccc5c4c3)cc2-c2oc(-c3ccccc3)c3ccccc23)cc1]

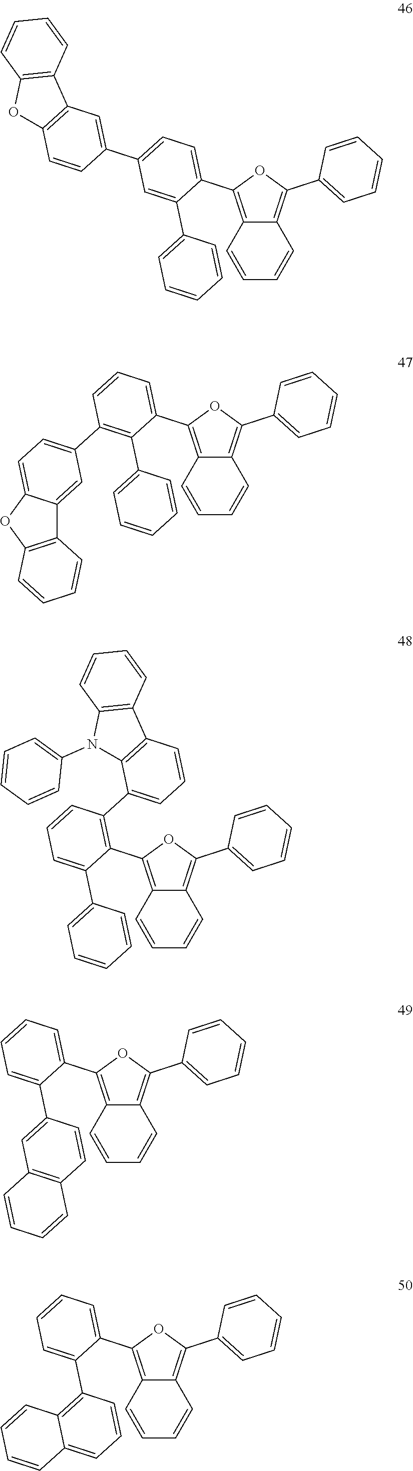 [SMILES: c1ccc(-c2cc(-c3ccc4oc5ccccc5c4c3)ccc2-c2oc(-c3ccccc3)c3ccccc23)cc1 ; c1ccc(-c2c(-c3ccc4oc5ccccc5c4c3)cccc2-c2oc(-c3ccccc3)c3ccccc23)cc1 ; c1ccc(-c2cccc(-c3cccc4c5ccccc5n(-c5ccccc5)c34)c2-c2oc(-c3ccccc3)c3ccccc23)cc1 ; c1ccc(-c2oc(-c3ccccc3-c3ccc4ccccc4c3)c3ccccc23)cc1 ; c1ccc(-c2oc(-c3ccccc3-c3cccc4ccccc34)c3ccccc23)cc1]

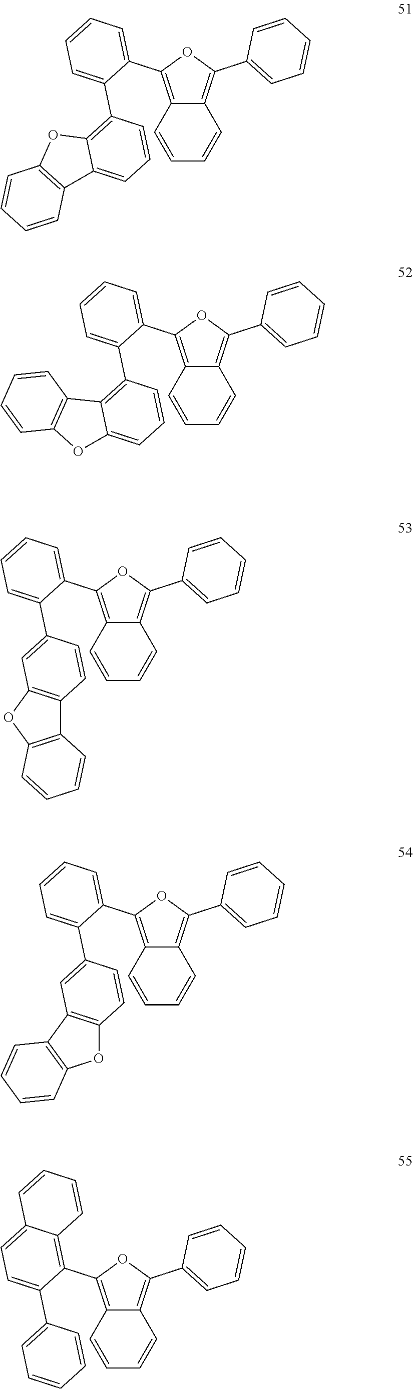 [SMILES: c1ccc(-c2oc(-c3ccccc3-c3cccc4c3oc3ccccc34)c3ccccc23)cc1 ; c1ccc(-c2oc(-c3ccccc3-c3cccc4oc5ccccc5c34)c3ccccc23)cc1 ; c1ccc(-c2oc(-c3ccccc3-c3ccc4c(c3)oc3ccccc34)c3ccccc23)cc1 ; c1ccc(-c2oc(-c3ccccc3-c3ccc4oc5ccccc5c4c3)c3ccccc23)cc1 ; c1ccc(-c2ccc3ccccc3c2-c2oc(-c3ccccc3)c3ccccc23)cc1]

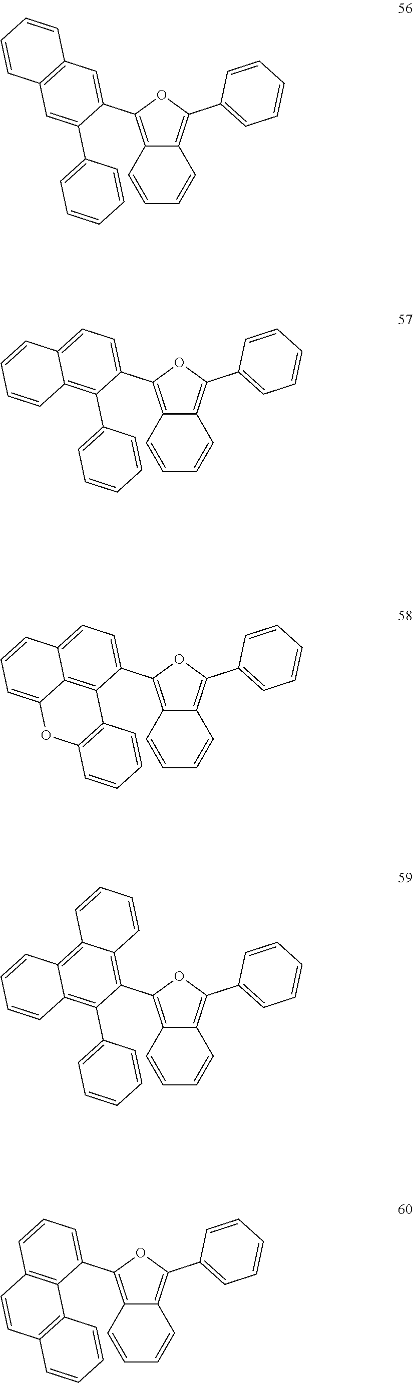 [SMILES: c1ccc(-c2cc3ccccc3cc2-c2oc(-c3ccccc3)c3ccccc23)cc1 ; c1ccc(-c2c(-c3oc(-c4ccccc4)c4ccccc34)ccc3ccccc23)cc1 ; c1ccc(-c2oc(-c3ccc4cccc5c4c3-c3ccccc3O5)c3ccccc23)cc1 ; c1ccc(-c2oc(-c3c(-c4ccccc4)c4ccccc4c4ccccc34)c3ccccc23)cc1 ; c1ccc(-c2oc(-c3cccc4ccc5ccccc5c34)c3ccccc23)cc1]

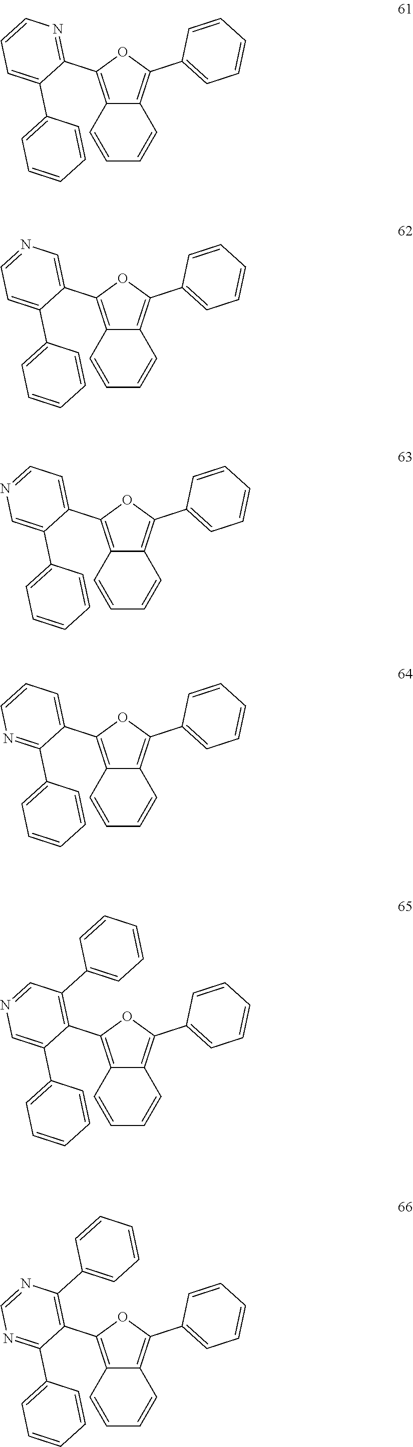 [SMILES: c1ccc(-c2cccnc2-c2oc(-c3ccccc3)c3ccccc23)cc1 ; c1ccc(-c2ccncc2-c2oc(-c3ccccc3)c3ccccc23)cc1 ; c1ccc(-c2cnccc2-c2oc(-c3ccccc3)c3ccccc23)cc1 ; c1ccc(-c2ncccc2-c2oc(-c3ccccc3)c3ccccc23)cc1 ; c1ccc(-c2cncc(-c3ccccc3)c2-c2oc(-c3ccccc3)c3ccccc23)cc1 ; c1ccc(-c2ncnc(-c3ccccc3)c2-c2oc(-c3ccccc3)c3ccccc23)cc1]

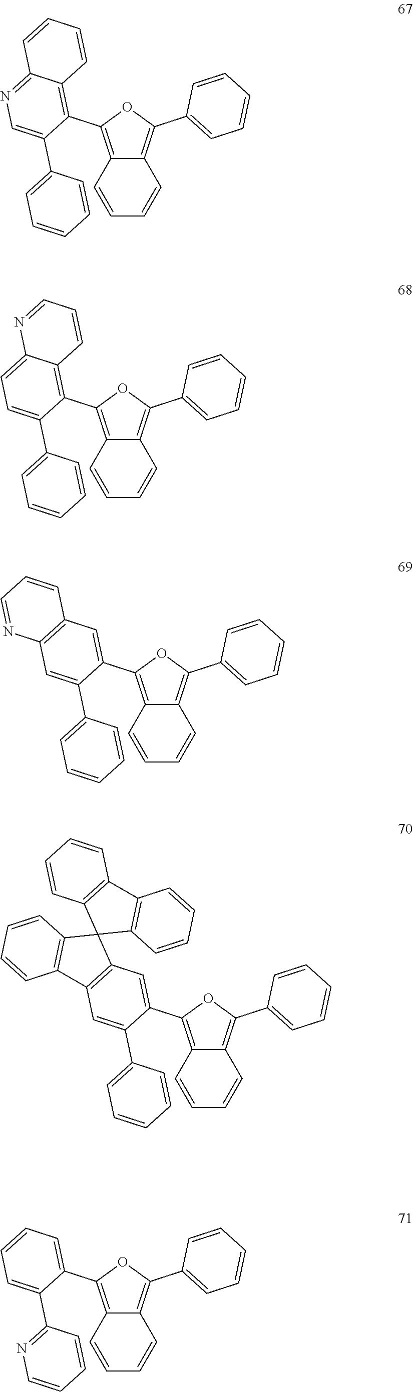 [SMILES: c1ccc(-c2cnc3ccccc3c2-c2oc(-c3ccccc3)c3ccccc23)cc1 ; c1ccc(-c2ccc3ncccc3c2-c2oc(-c3ccccc3)c3ccccc23)cc1 ; c1ccc(-c2cc3ncccc3cc2-c2oc(-c3ccccc3)c3ccccc23)cc1 ; c1ccc(-c2cc3c(cc2-c2oc(-c4ccccc4)c4ccccc24)C2(c4ccccc4-c4ccccc42)c2ccccc2-3)cc1 ; c1ccc(-c2oc(-c3ccccc3-c3ccccn3)c3ccccc23)cc1]

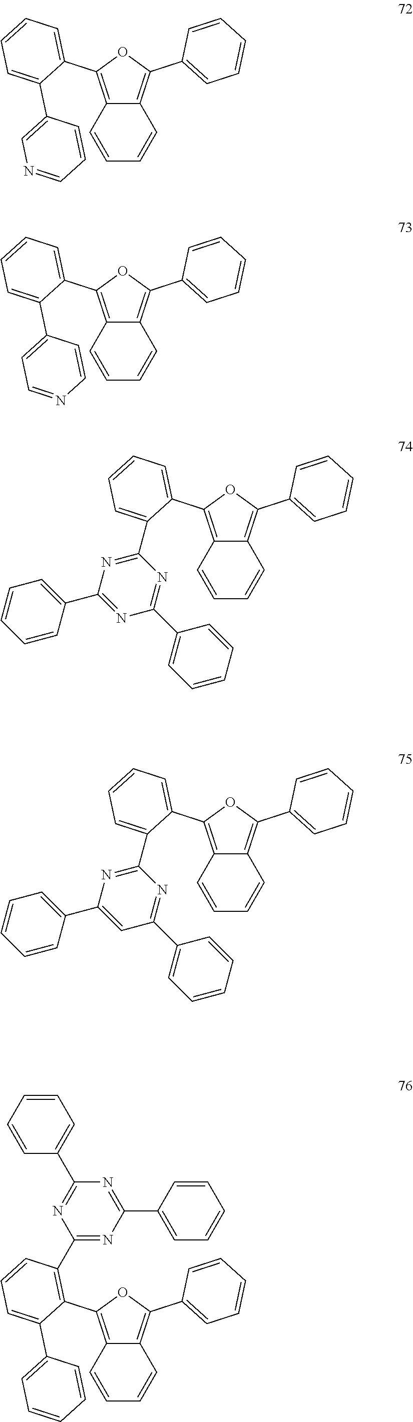 [SMILES: c1ccc(-c2oc(-c3ccccc3-c3cccnc3)c3ccccc23)cc1 ; c1ccc(-c2oc(-c3ccccc3-c3ccncc3)c3ccccc23)cc1 ; c1ccc(-c2nc(-c3ccccc3)nc(-c3ccccc3-c3oc(-c4ccccc4)c4ccccc34)n2)cc1 ; c1ccc(-c2cc(-c3ccccc3)nc(-c3ccccc3-c3oc(-c4ccccc4)c4ccccc34)n2)cc1 ; c1ccc(-c2nc(-c3ccccc3)nc(-c3cccc(-c4ccccc4)c3-c3oc(-c4ccccc4)c4ccccc34)n2)cc1]

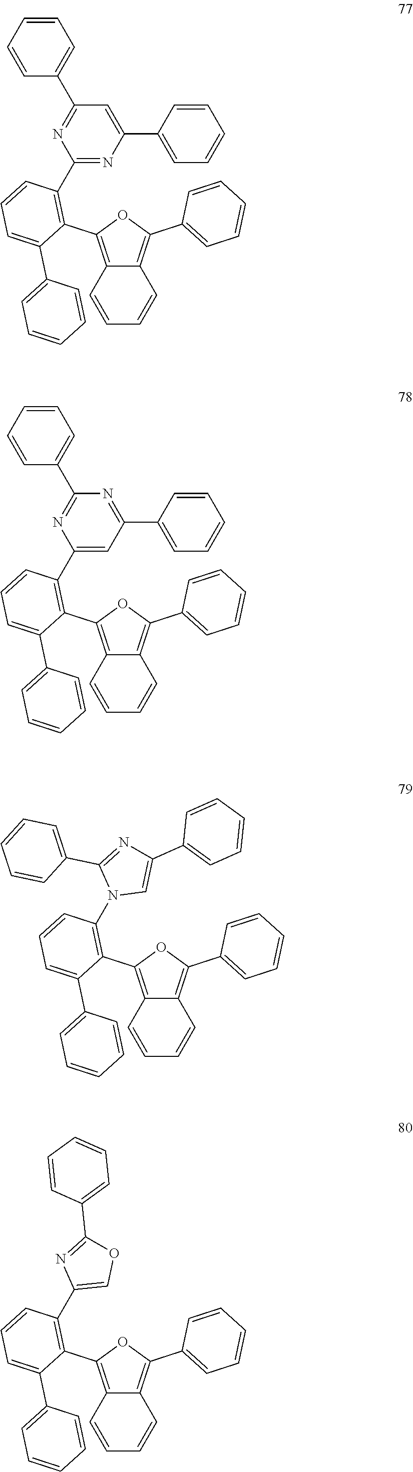 [SMILES: c1ccc(-c2cc(-c3ccccc3)nc(-c3cccc(-c4ccccc4)c3-c3oc(-c4ccccc4)c4ccccc34)n2)cc1 ; c1ccc(-c2cc(-c3cccc(-c4ccccc4)c3-c3oc(-c4ccccc4)c4ccccc34)nc(-c3ccccc3)n2)cc1 ; c1ccc(-c2cn(-c3cccc(-c4ccccc4)c3-c3oc(-c4ccccc4)c4ccccc34)c(-c3ccccc3)n2)cc1 ; c1ccc(-c2nc(-c3cccc(-c4ccccc4)c3-c3oc(-c4ccccc4)c4ccccc34)co2)cc1]

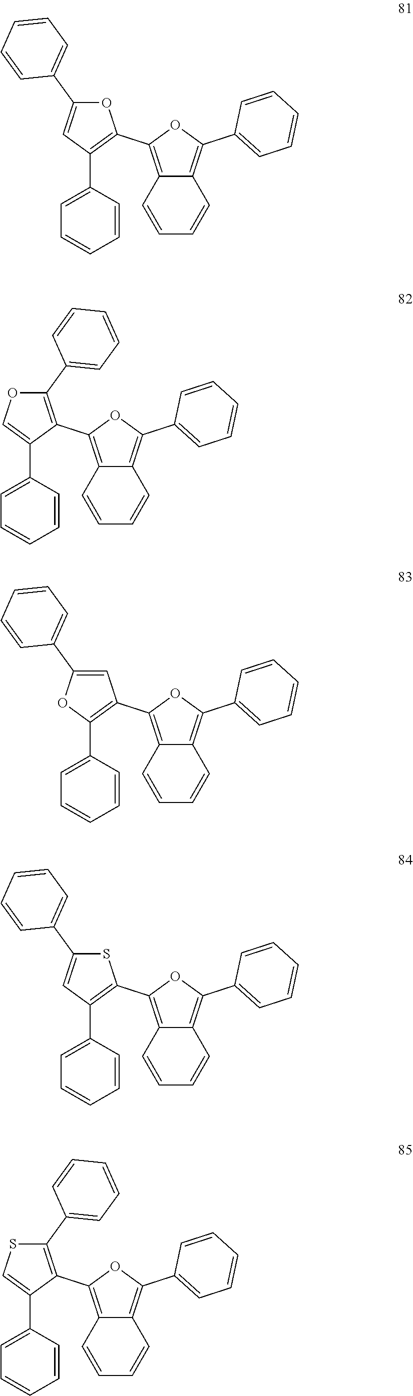

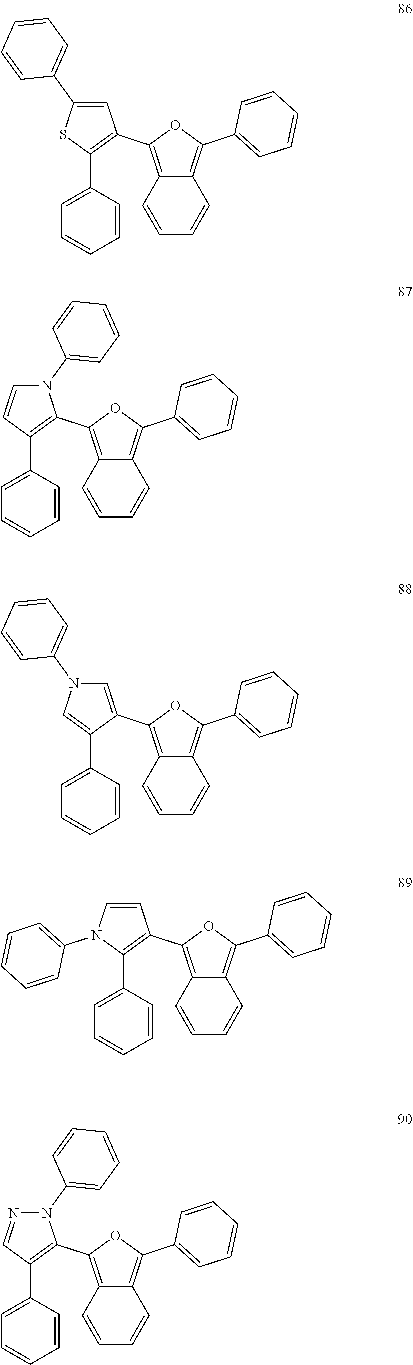 [SMILES: c1ccc(-c2cc(-c3oc(-c4ccccc4)c4ccccc34)c(-c3ccccc3)s2)cc1 ; c1ccc(-c2ccn(-c3ccccc3)c2-c2oc(-c3ccccc3)c3ccccc23)cc1 ; c1ccc(-c2cn(-c3ccccc3)cc2-c2oc(-c3ccccc3)c3ccccc23)cc1 ; c1ccc(-c2oc(-c3ccn(-c4ccccc4)c3-c3ccccc3)c3ccccc23)cc1 ; c1ccc(-c2cnn(-c3ccccc3)c2-c2oc(-c3ccccc3)c3ccccc23)cc1]

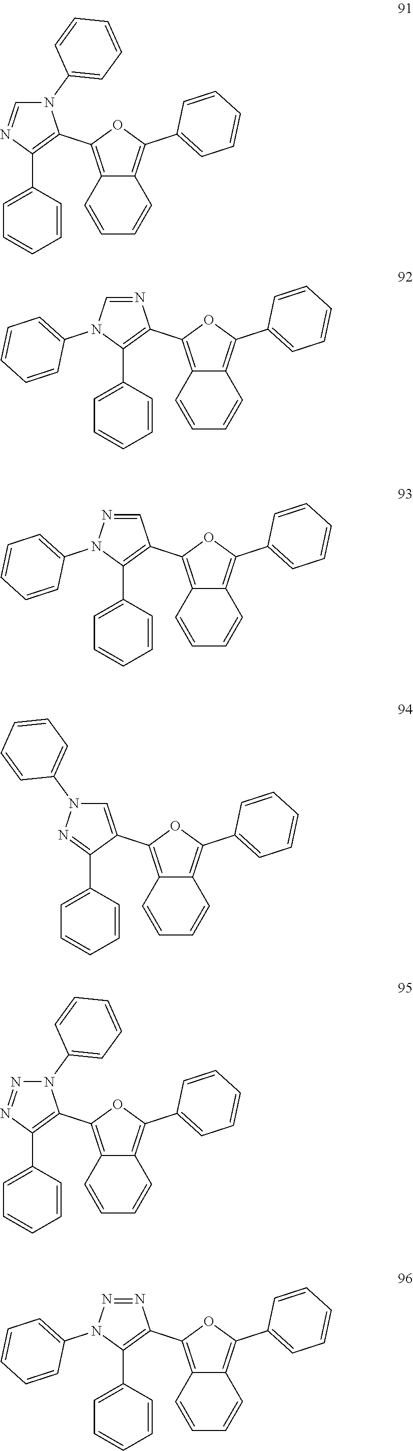

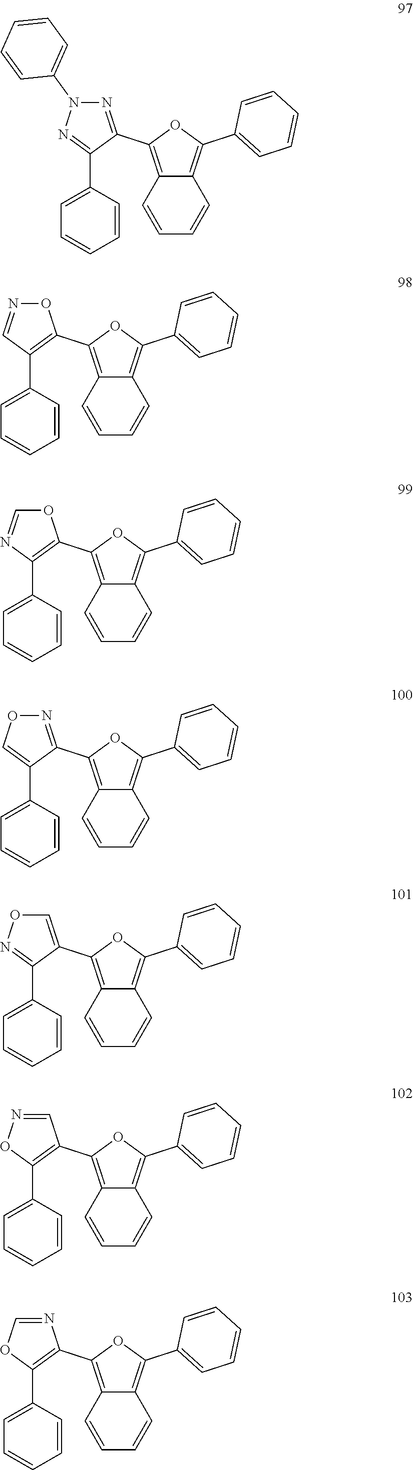

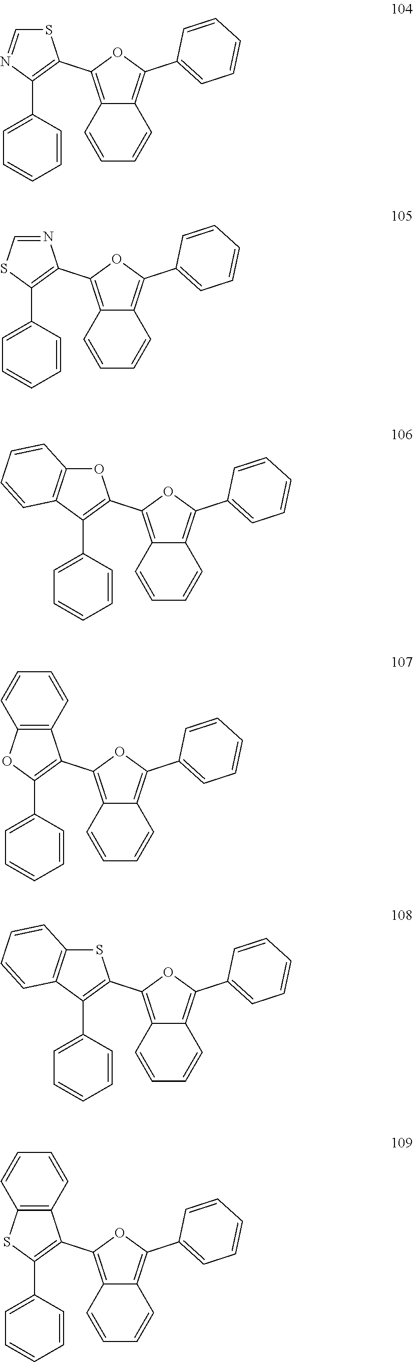

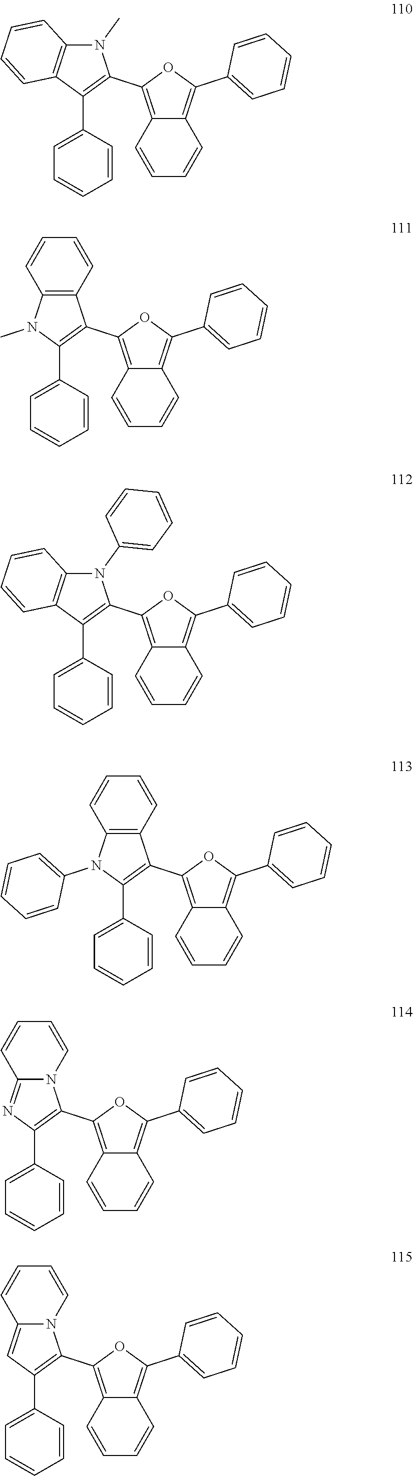

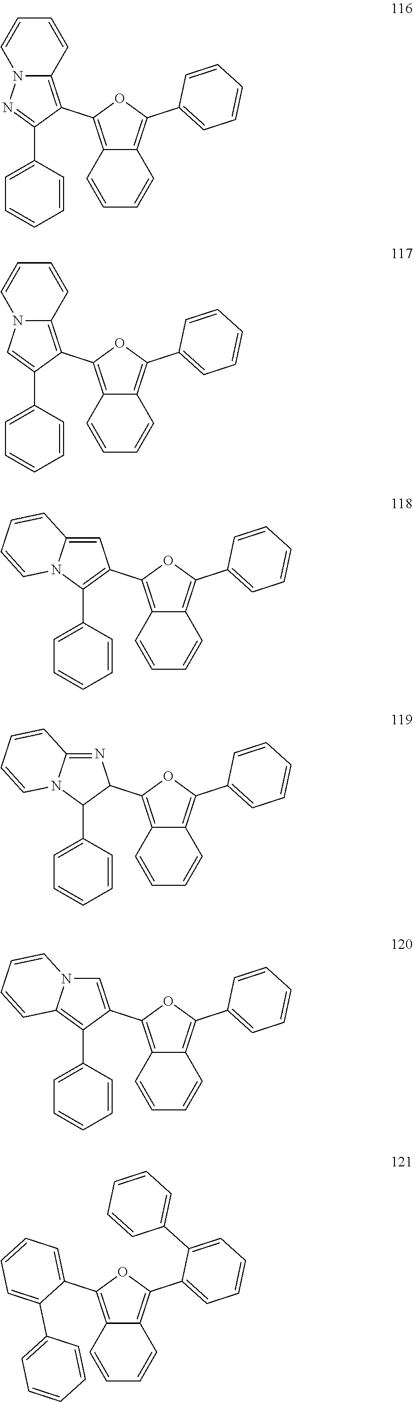

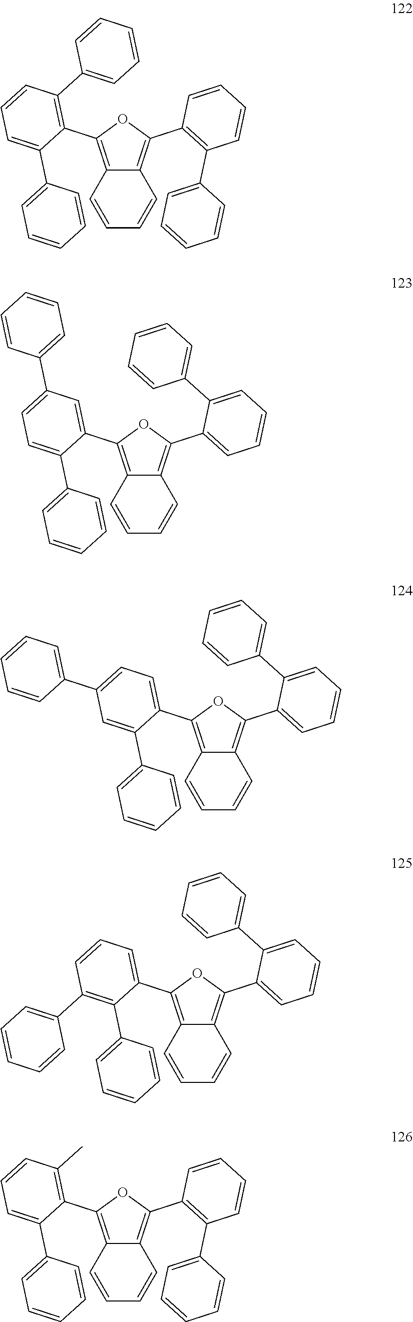 [SMILES: c1ccc(-c2ccccc2-c2oc(-c3c(-c4ccccc4)cccc3-c3ccccc3)c3ccccc23)cc1 ; c1ccc(-c2ccc(-c3ccccc3)c(-c3oc(-c4ccccc4-c4ccccc4)c4ccccc34)c2)cc1 ; c1ccc(-c2ccc(-c3oc(-c4ccccc4-c4ccccc4)c4ccccc34)c(-c3ccccc3)c2)cc1 ; c1ccc(-c2ccccc2-c2oc(-c3cccc(-c4ccccc4)c3-c3ccccc3)c3ccccc23)cc1 ; Cc1cccc(-c2ccccc2)c1-c1oc(-c2ccccc2-c2ccccc2)c2ccccc12]

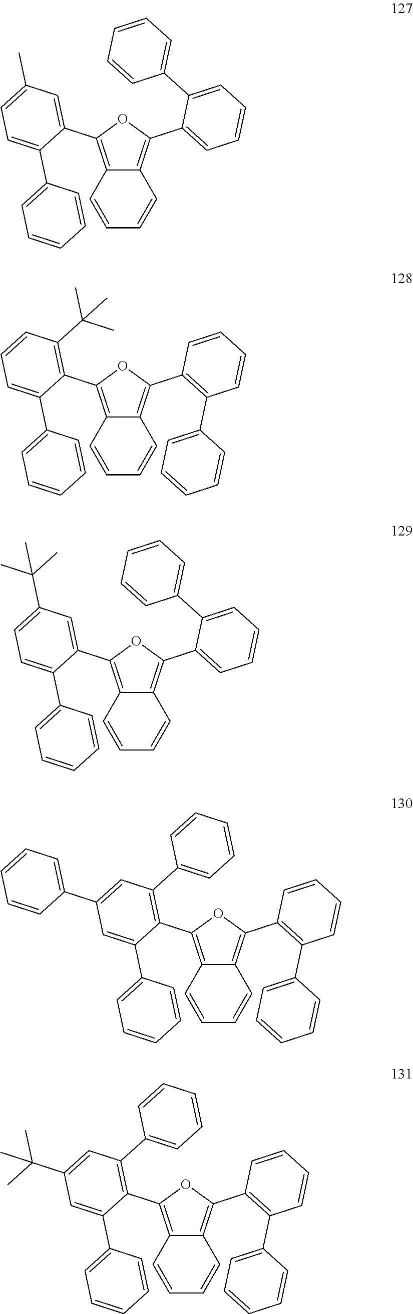

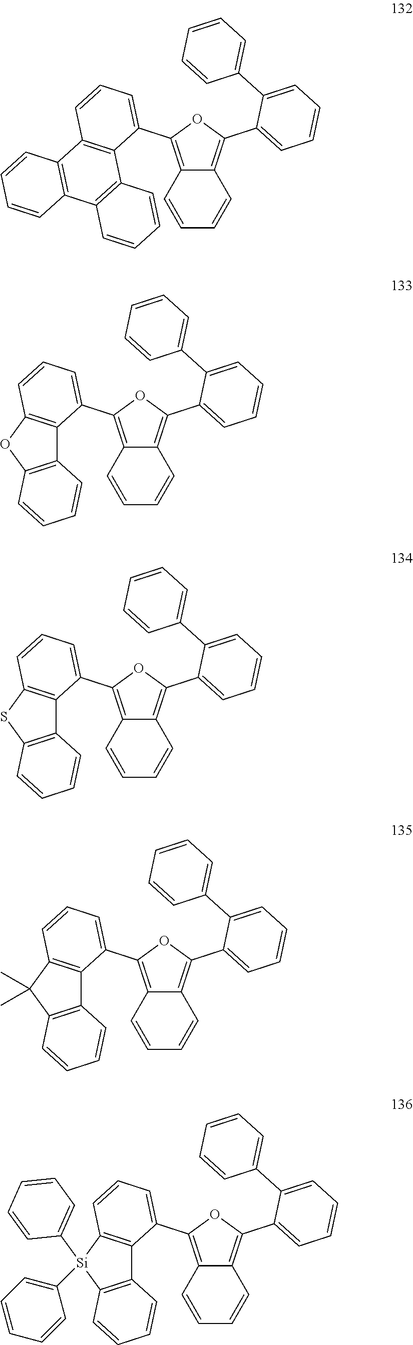 [SMILES: c1ccc(-c2ccccc2-c2oc(-c3cccc4c5ccccc5c5ccccc5c34)c3ccccc23)cc1 ; c1ccc(-c2ccccc2-c2oc(-c3cccc4oc5ccccc5c34)c3ccccc23)cc1 ; c1ccc(-c2ccccc2-c2oc(-c3cccc4sc5ccccc5c34)c3ccccc23)cc1 ; CC1(C)c2ccccc2-c2c(-c3oc(-c4ccccc4-c4ccccc4)c4ccccc34)cccc21 ; c1ccc(-c2ccccc2-c2oc(-c3cccc4c3-c3ccccc3[Si]4(c3ccccc3)c3ccccc3)c3ccccc23)cc1]

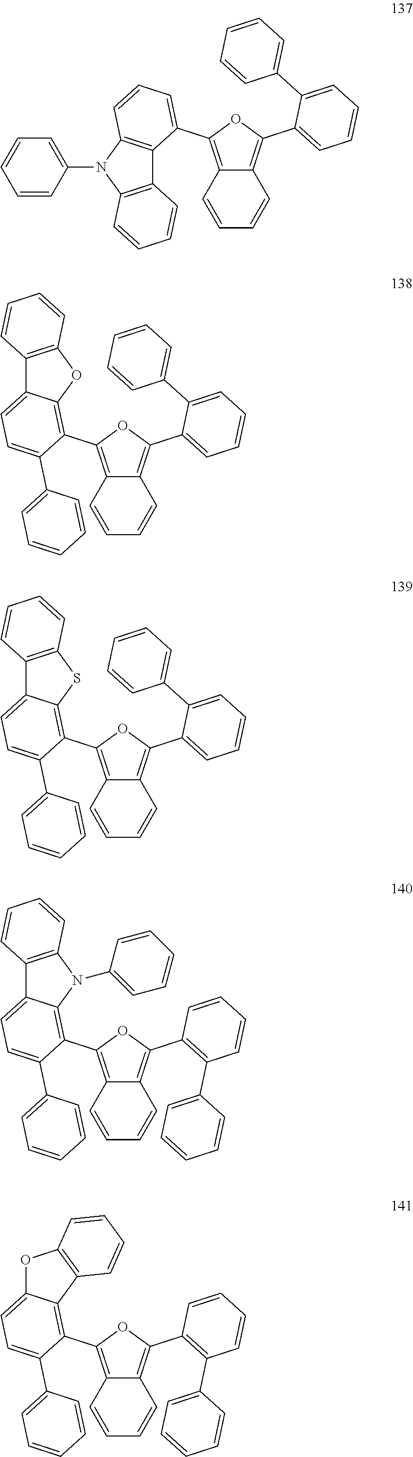

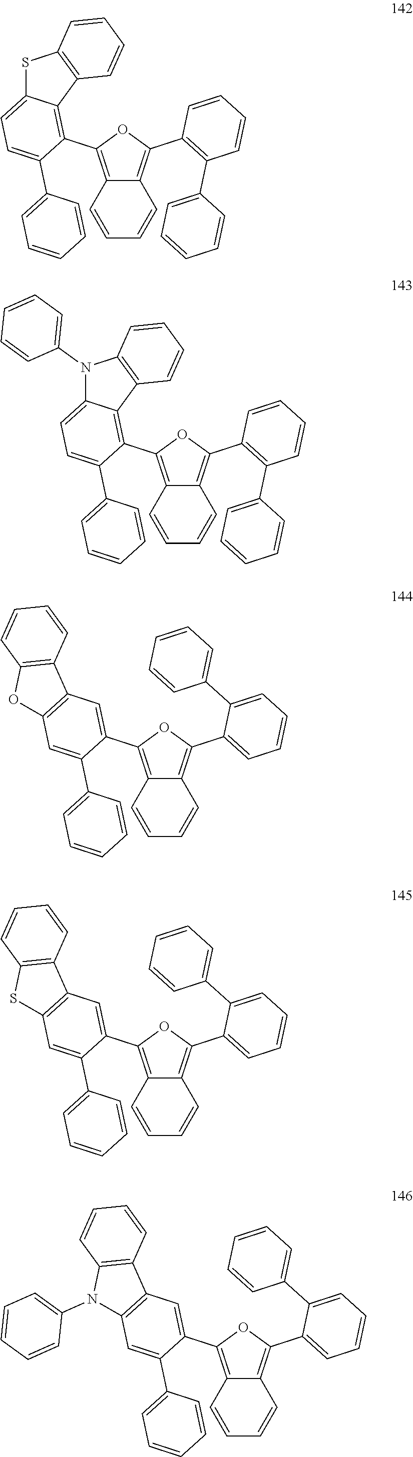

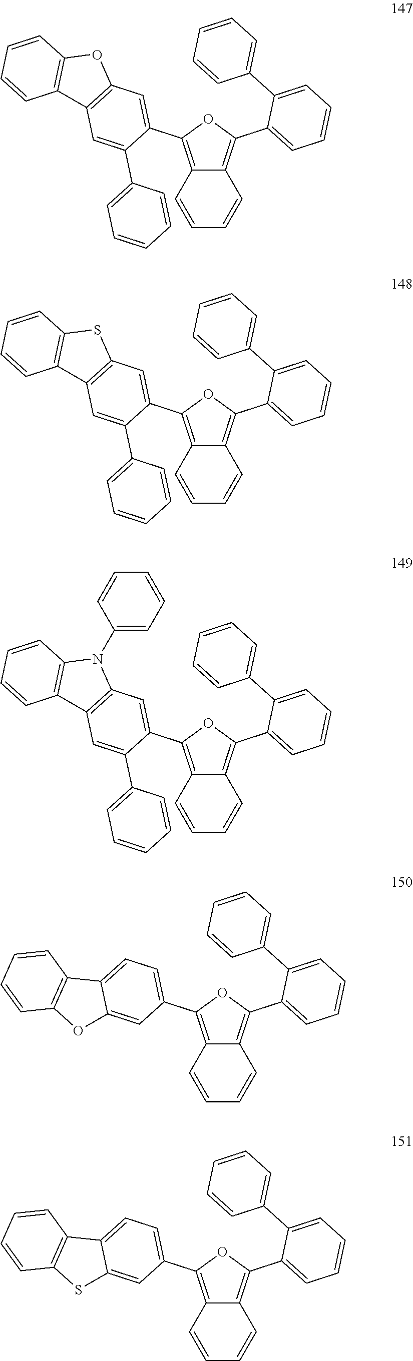 [SMILES: c1ccc(-c2ccccc2-c2oc(-c3cc4oc5ccccc5c4cc3-c3ccccc3)c3ccccc23)cc1 ; c1ccc(-c2ccccc2-c2oc(-c3cc4sc5ccccc5c4cc3-c3ccccc3)c3ccccc23)cc1 ; c1ccc(-c2ccccc2-c2oc(-c3cc4c(cc3-c3ccccc3)c3ccccc3n4-c3ccccc3)c3ccccc23)cc1 ; c1ccc(-c2ccccc2-c2oc(-c3ccc4c(c3)oc3ccccc34)c3ccccc23)cc1 ; c1ccc(-c2ccccc2-c2oc(-c3ccc4c(c3)sc3ccccc34)c3ccccc23)cc1]

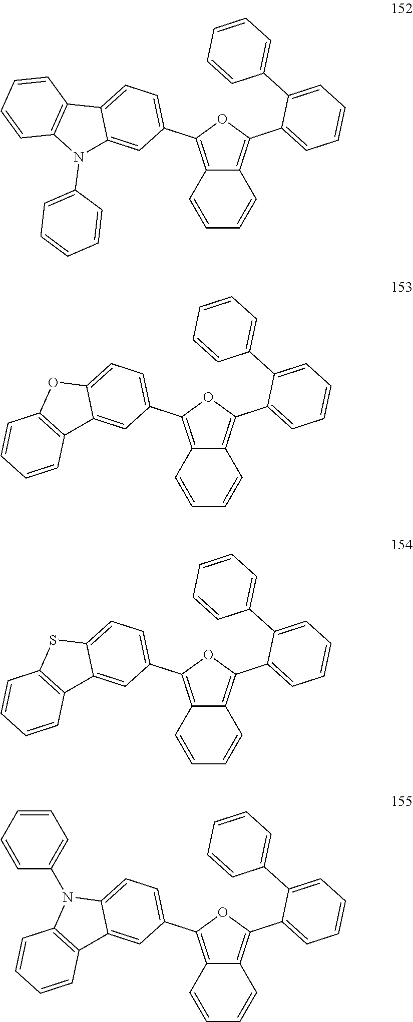 [SMILES: c1ccc(-c2ccccc2-c2oc(-c3ccc4c5ccccc5n(-c5ccccc5)c4c3)c3ccccc23)cc1 ; c1ccc(-c2ccccc2-c2oc(-c3ccc4oc5ccccc5c4c3)c3ccccc23)cc1 ; c1ccc(-c2ccccc2-c2oc(-c3ccc4sc5ccccc5c4c3)c3ccccc23)cc1 ; c1ccc(-c2ccccc2-c2oc(-c3ccc4c(c3)c3ccccc3n4-c3ccccc3)c3ccccc23)cc1]

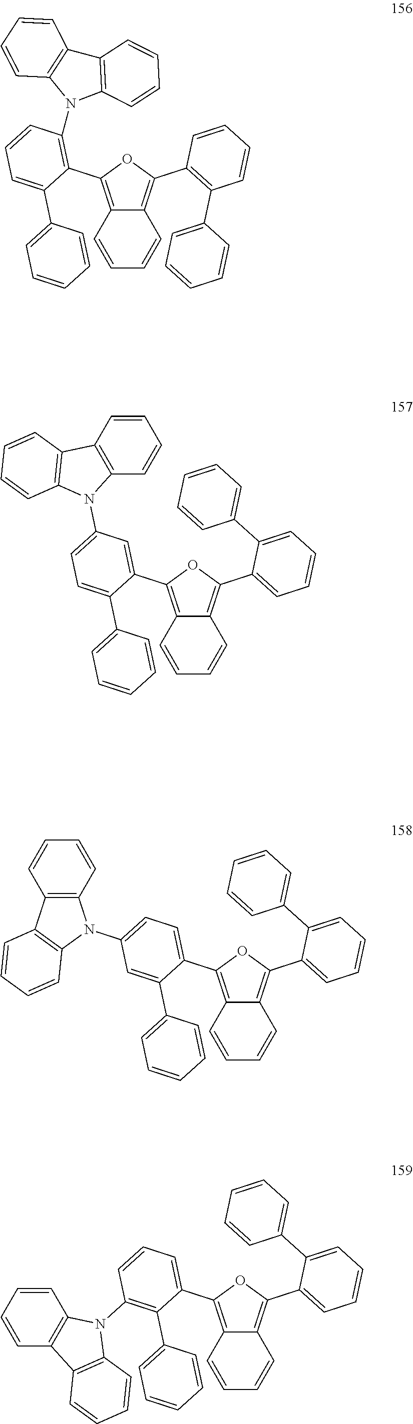 [SMILES: c1ccc(-c2ccccc2-c2oc(-c3c(-c4ccccc4)cccc3-n3c4ccccc4c4ccccc43)c3ccccc23)cc1 ; c1ccc(-c2ccccc2-c2oc(-c3cc(-n4c5ccccc5c5ccccc54)ccc3-c3ccccc3)c3ccccc23)cc1 ; c1ccc(-c2ccccc2-c2oc(-c3ccc(-n4c5ccccc5c5ccccc54)cc3-c3ccccc3)c3ccccc23)cc1 ; c1ccc(-c2ccccc2-c2oc(-c3cccc(-n4c5ccccc5c5ccccc54)c3-c3ccccc3)c3ccccc23)cc1]

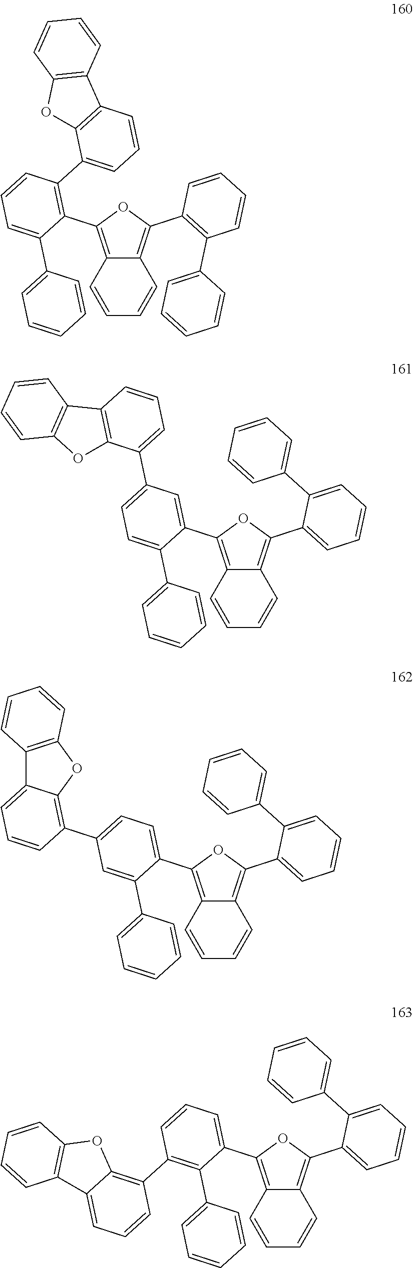

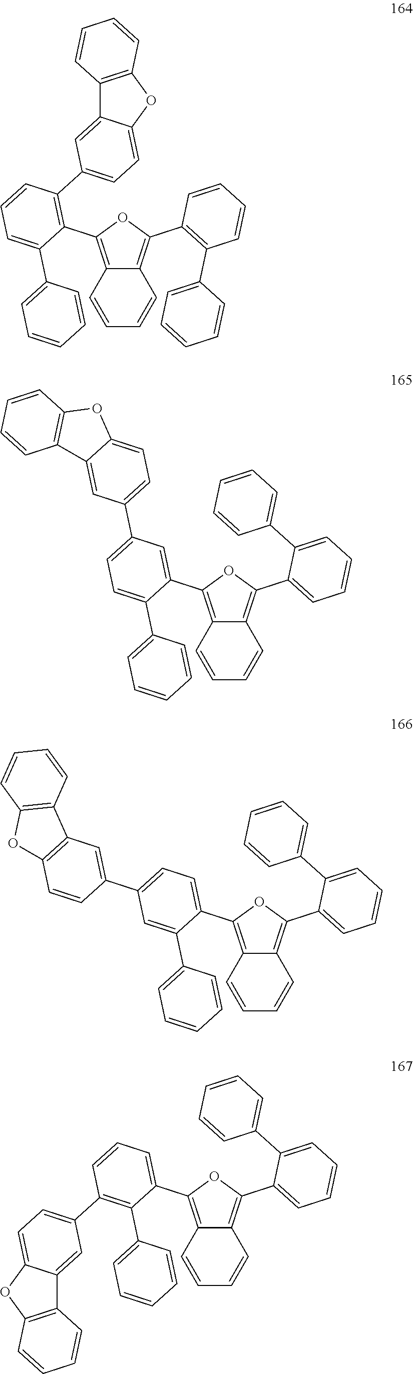 [SMILES: c1ccc(-c2ccccc2-c2oc(-c3c(-c4ccccc4)cccc3-c3ccc4oc5ccccc5c4c3)c3ccccc23)cc1 ; c1ccc(-c2ccccc2-c2oc(-c3cc(-c4ccc5oc6ccccc6c5c4)ccc3-c3ccccc3)c3ccccc23)cc1 ; c1ccc(-c2ccccc2-c2oc(-c3ccc(-c4ccc5oc6ccccc6c5c4)cc3-c3ccccc3)c3ccccc23)cc1 ; c1ccc(-c2ccccc2-c2oc(-c3cccc(-c4ccc5oc6ccccc6c5c4)c3-c3ccccc3)c3ccccc23)cc1]

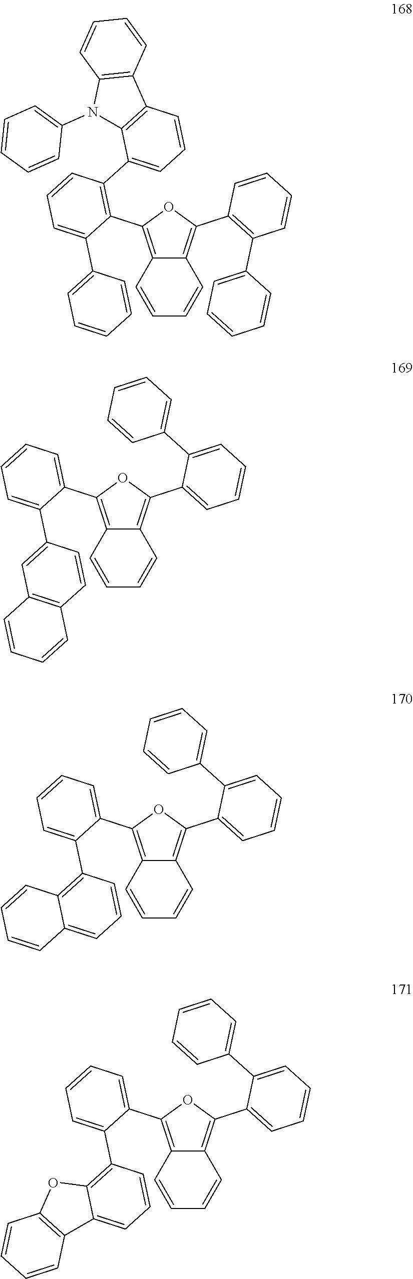 [SMILES: c1ccc(-c2ccccc2-c2oc(-c3c(-c4ccccc4)cccc3-c3cccc4c5ccccc5n(-c5ccccc5)c34)c3ccccc23)cc1 ; c1ccc(-c2ccccc2-c2oc(-c3ccccc3-c3ccc4ccccc4c3)c3ccccc23)cc1 ; c1ccc(-c2ccccc2-c2oc(-c3ccccc3-c3cccc4ccccc34)c3ccccc23)cc1 ; c1ccc(-c2ccccc2-c2oc(-c3ccccc3-c3cccc4c3oc3ccccc34)c3ccccc23)cc1]

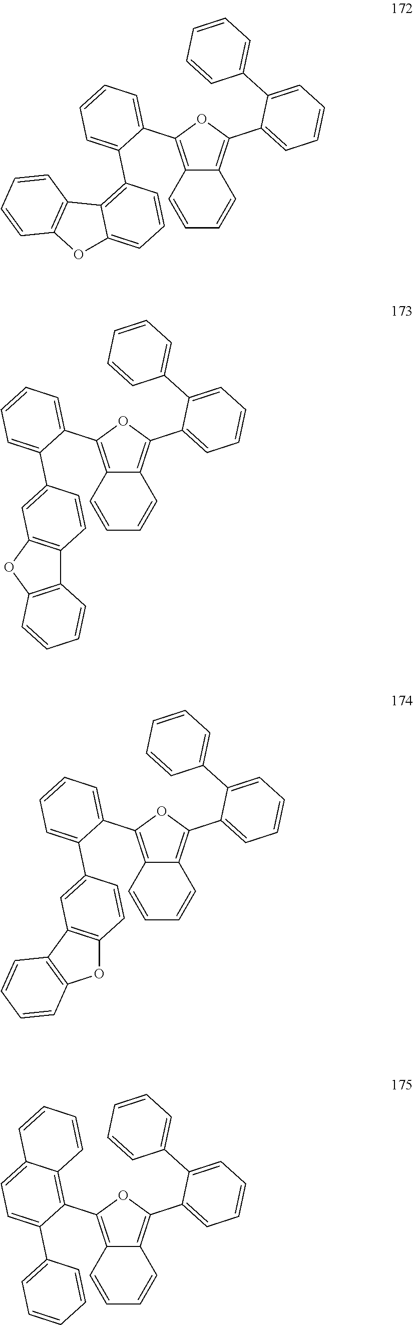 [SMILES: c1ccc(-c2ccccc2-c2oc(-c3ccccc3-c3cccc4oc5ccccc5c34)c3ccccc23)cc1 ; c1ccc(-c2ccccc2-c2oc(-c3ccccc3-c3ccc4c(c3)oc3ccccc34)c3ccccc23)cc1 ; c1ccc(-c2ccccc2-c2oc(-c3ccccc3-c3ccc4oc5ccccc5c4c3)c3ccccc23)cc1 ; c1ccc(-c2ccccc2-c2oc(-c3c(-c4ccccc4)ccc4ccccc34)c3ccccc23)cc1]

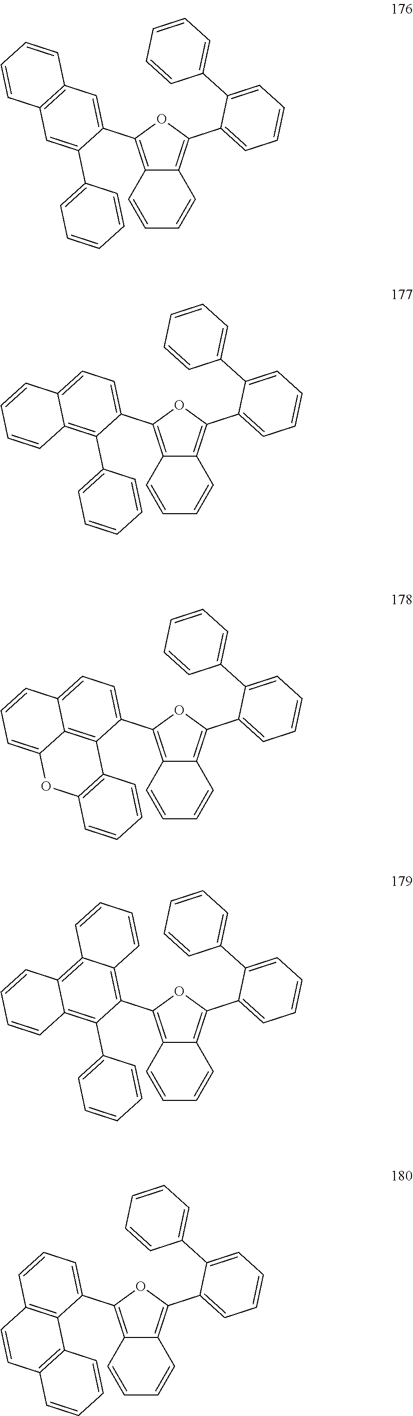 [SMILES: c1ccc(-c2ccccc2-c2oc(-c3cc4ccccc4cc3-c3ccccc3)c3ccccc23)cc1 ; c1ccc(-c2ccccc2-c2oc(-c3ccc4ccccc4c3-c3ccccc3)c3ccccc23)cc1 ; c1ccc(-c2ccccc2-c2oc(-c3ccc4cccc5c4c3-c3ccccc3O5)c3ccccc23)cc1 ; c1ccc(-c2ccccc2-c2oc(-c3c(-c4ccccc4)c4ccccc4c4ccccc34)c3ccccc23)cc1 ; c1ccc(-c2ccccc2-c2oc(-c3cccc4ccc5ccccc5c34)c3ccccc23)cc1]

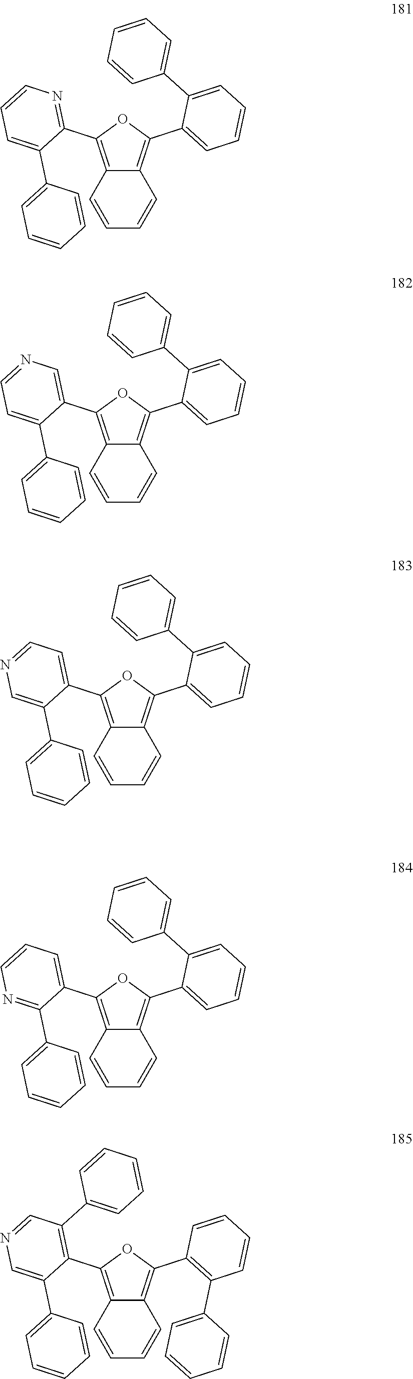 [SMILES: c1ccc(-c2ccccc2-c2oc(-c3ncccc3-c3ccccc3)c3ccccc23)cc1 ; c1ccc(-c2ccccc2-c2oc(-c3cnccc3-c3ccccc3)c3ccccc23)cc1 ; c1ccc(-c2ccccc2-c2oc(-c3ccncc3-c3ccccc3)c3ccccc23)cc1 ; c1ccc(-c2ccccc2-c2oc(-c3cccnc3-c3ccccc3)c3ccccc23)cc1 ; c1ccc(-c2ccccc2-c2oc(-c3c(-c4ccccc4)cncc3-c3ccccc3)c3ccccc23)cc1]

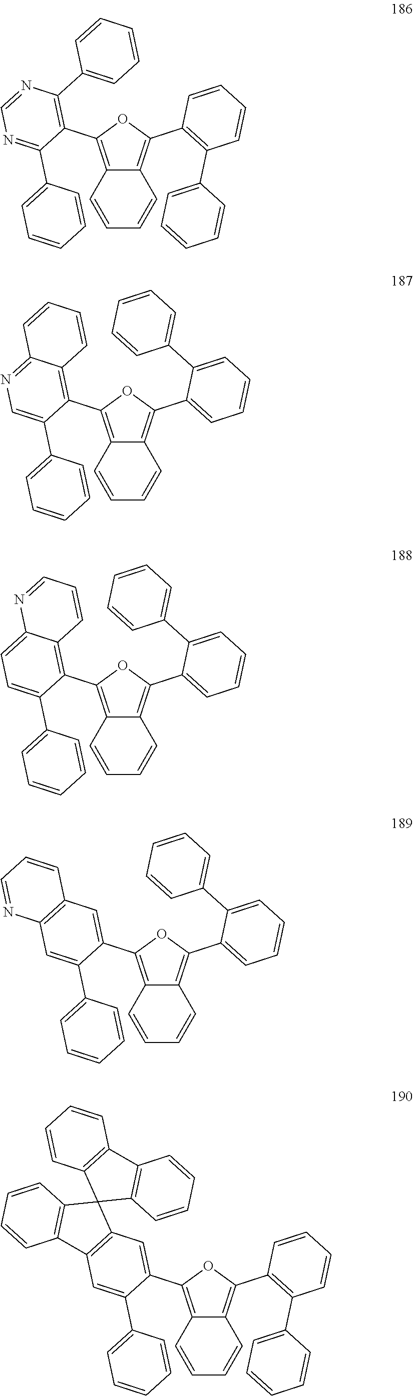 [SMILES: c1ccc(-c2ccccc2-c2oc(-c3c(-c4ccccc4)ncnc3-c3ccccc3)c3ccccc23)cc1 ; c1ccc(-c2ccccc2-c2oc(-c3c(-c4ccccc4)cnc4ccccc34)c3ccccc23)cc1 ; c1ccc(-c2ccccc2-c2oc(-c3c(-c4ccccc4)ccc4ncccc34)c3ccccc23)cc1 ; c1ccc(-c2ccccc2-c2oc(-c3cc4cccnc4cc3-c3ccccc3)c3ccccc23)cc1 ; c1ccc(-c2ccccc2-c2oc(-c3cc4c(cc3-c3ccccc3)-c3ccccc3C43c4ccccc4-c4ccccc43)c3ccccc23)cc1]

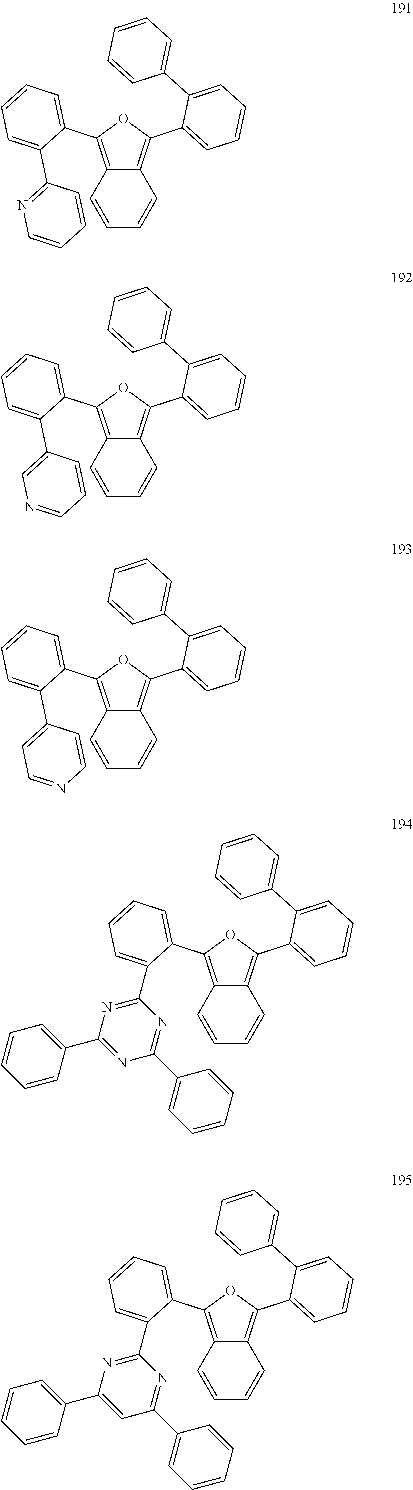 [SMILES: c1ccc(-c2ccccc2-c2oc(-c3ccccc3-c3ccccn3)c3ccccc23)cc1 ; c1ccc(-c2ccccc2-c2oc(-c3ccccc3-c3cccnc3)c3ccccc23)cc1 ; c1ccc(-c2ccccc2-c2oc(-c3ccccc3-c3ccncc3)c3ccccc23)cc1 ; c1ccc(-c2nc(-c3ccccc3)nc(-c3ccccc3-c3oc(-c4ccccc4-c4ccccc4)c4ccccc34)n2)cc1 ; c1ccc(-c2cc(-c3ccccc3)nc(-c3ccccc3-c3oc(-c4ccccc4-c4ccccc4)c4ccccc34)n2)cc1]

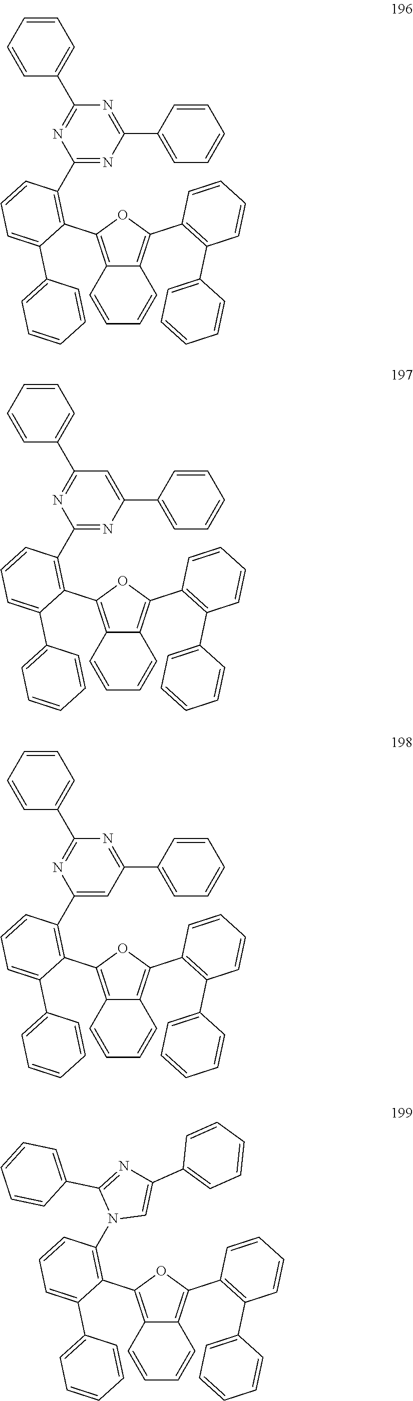

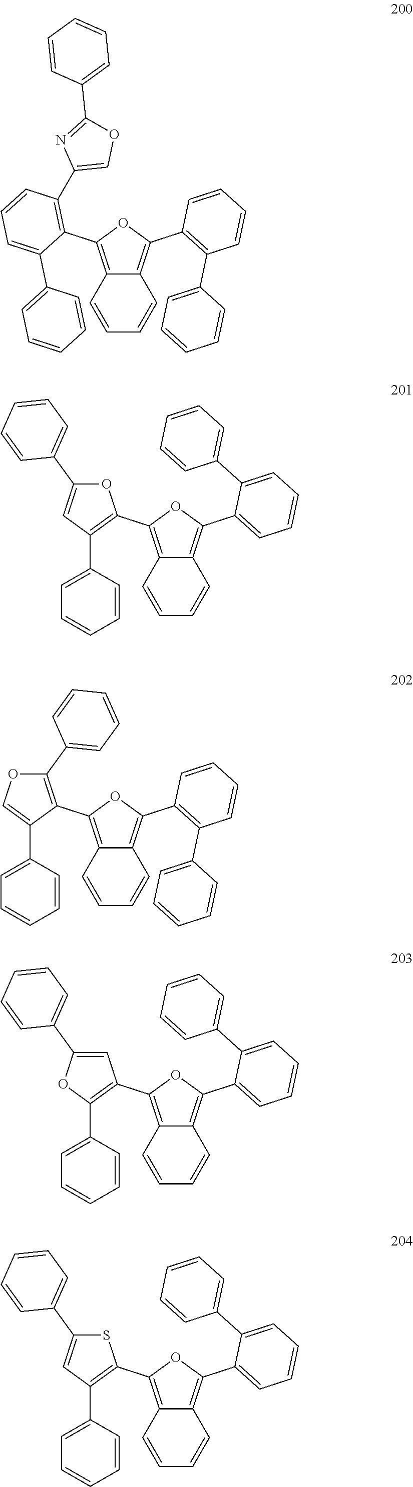

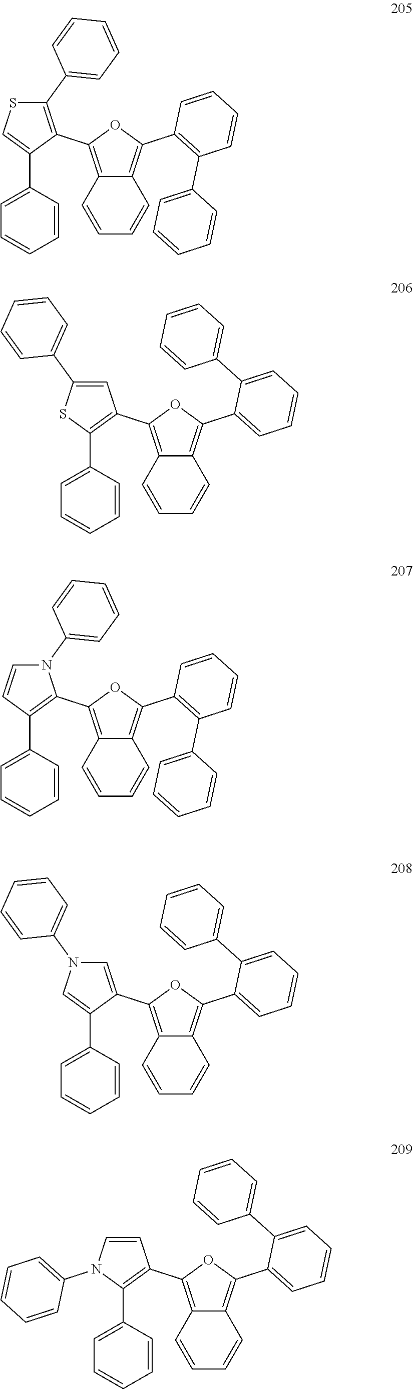 [SMILES: c1ccc(-c2ccccc2-c2oc(-c3c(-c4ccccc4)csc3-c3ccccc3)c3ccccc23)cc1 ; c1ccc(-c2cc(-c3oc(-c4ccccc4-c4ccccc4)c4ccccc34)c(-c3ccccc3)s2)cc1 ; c1ccc(-c2ccccc2-c2oc(-c3c(-c4ccccc4)ccn3-c3ccccc3)c3ccccc23)cc1 ; c1ccc(-c2ccccc2-c2oc(-c3cn(-c4ccccc4)cc3-c3ccccc3)c3ccccc23)cc1 ; c1ccc(-c2ccccc2-c2oc(-c3ccn(-c4ccccc4)c3-c3ccccc3)c3ccccc23)cc1]

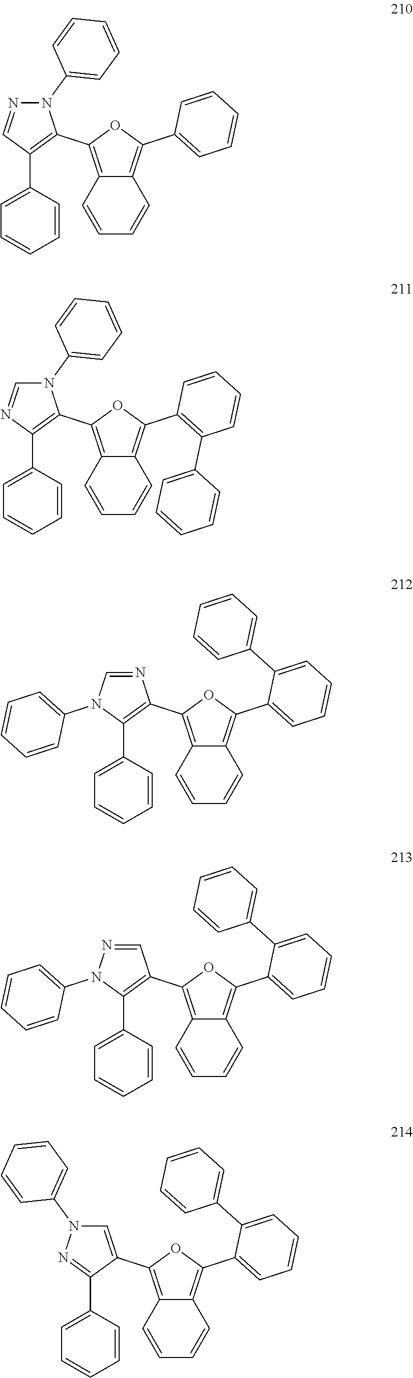 [SMILES: c1ccc(-c2cnn(-c3ccccc3)c2-c2oc(-c3ccccc3)c3ccccc23)cc1 ; c1ccc(-c2ccccc2-c2oc(-c3c(-c4ccccc4)ncn3-c3ccccc3)c3ccccc23)cc1 ; c1ccc(-c2ccccc2-c2oc(-c3ncn(-c4ccccc4)c3-c3ccccc3)c3ccccc23)cc1 ; c1ccc(-c2ccccc2-c2oc(-c3cnn(-c4ccccc4)c3-c3ccccc3)c3ccccc23)cc1 ; c1ccc(-c2ccccc2-c2oc(-c3cn(-c4ccccc4)nc3-c3ccccc3)c3ccccc23)cc1]

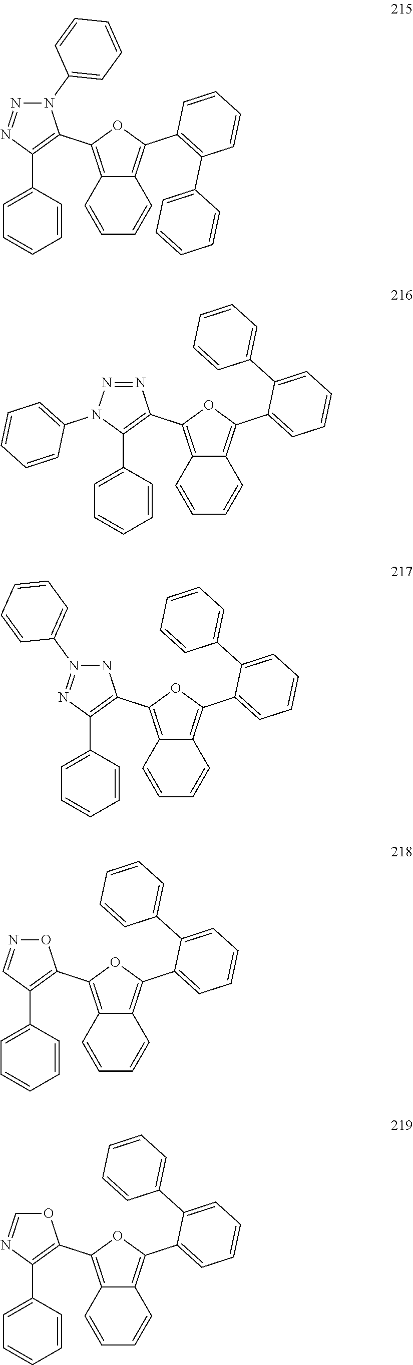

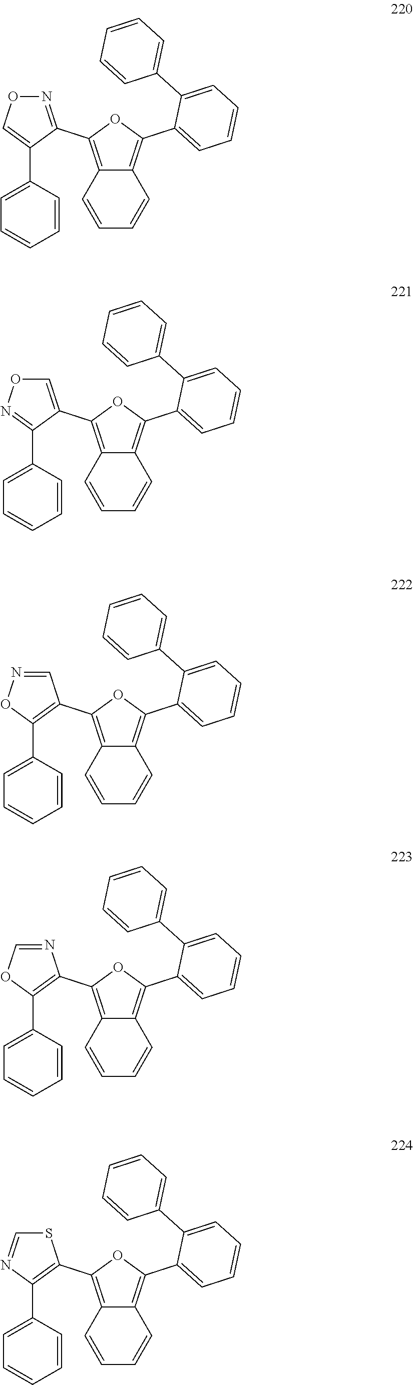 [SMILES: c1ccc(-c2ccccc2-c2oc(-c3nocc3-c3ccccc3)c3ccccc23)cc1 ; c1ccc(-c2ccccc2-c2oc(-c3conc3-c3ccccc3)c3ccccc23)cc1 ; c1ccc(-c2ccccc2-c2oc(-c3cnoc3-c3ccccc3)c3ccccc23)cc1 ; c1ccc(-c2ccccc2-c2oc(-c3ncoc3-c3ccccc3)c3ccccc23)cc1 ; c1ccc(-c2ccccc2-c2oc(-c3scnc3-c3ccccc3)c3ccccc23)cc1]

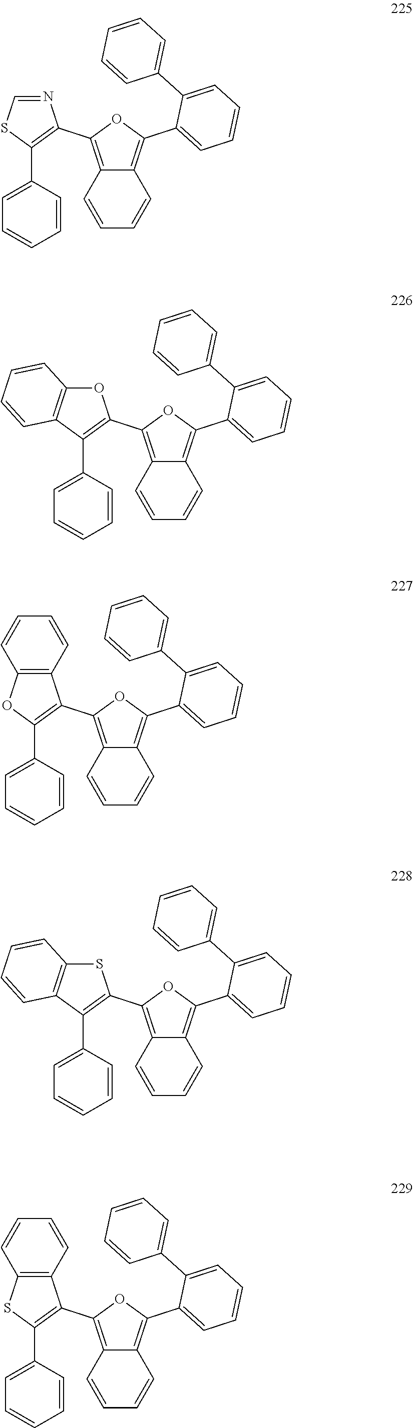 [SMILES: c1ccc(-c2ccccc2-c2oc(-c3ncsc3-c3ccccc3)c3ccccc23)cc1 ; c1ccc(-c2ccccc2-c2oc(-c3oc4ccccc4c3-c3ccccc3)c3ccccc23)cc1 ; c1ccc(-c2ccccc2-c2oc(-c3c(-c4ccccc4)oc4ccccc34)c3ccccc23)cc1 ; c1ccc(-c2ccccc2-c2oc(-c3sc4ccccc4c3-c3ccccc3)c3ccccc23)cc1 ; c1ccc(-c2ccccc2-c2oc(-c3c(-c4ccccc4)sc4ccccc34)c3ccccc23)cc1]

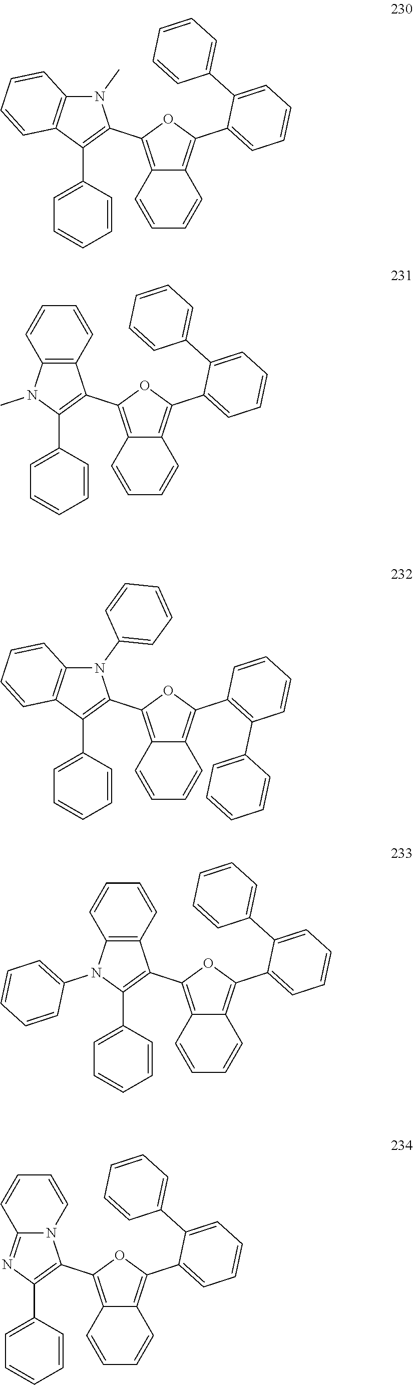

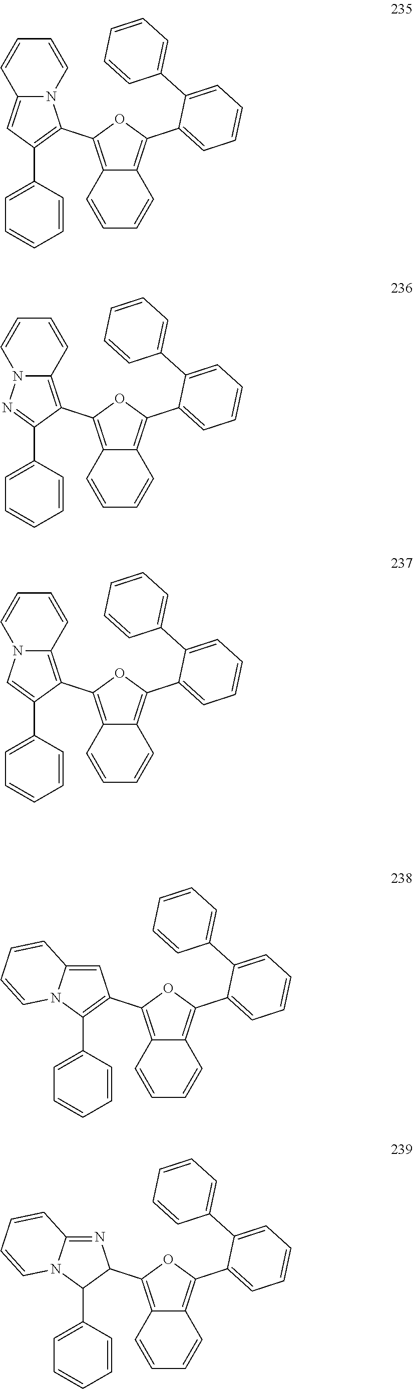 [SMILES: c1ccc(-c2ccccc2-c2oc(-c3c(-c4ccccc4)cc4ccccn34)c3ccccc23)cc1 ; c1ccc(-c2ccccc2-c2oc(-c3c(-c4ccccc4)nn4ccccc34)c3ccccc23)cc1 ; c1ccc(-c2ccccc2-c2oc(-c3c(-c4ccccc4)cn4ccccc34)c3ccccc23)cc1 ; c1ccc(-c2ccccc2-c2oc(-c3cc4ccccn4c3-c3ccccc3)c3ccccc23)cc1 ; C1=CC2=NC(c3oc(-c4ccccc4-c4ccccc4)c4ccccc34)C(c3ccccc3)N2C=C1]

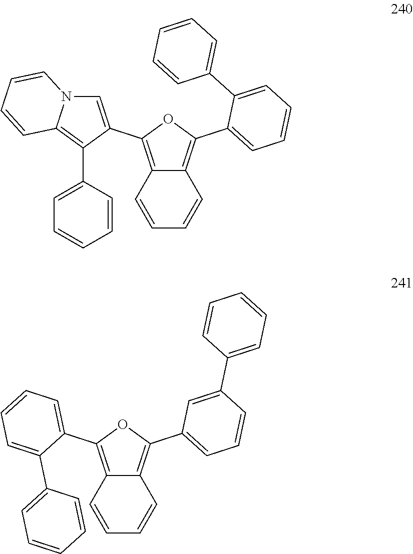 [SMILES: c1ccc(-c2ccccc2-c2oc(-c3cn4ccccc4c3-c3ccccc3)c3ccccc23)cc1 ; c1ccc(-c2cccc(-c3oc(-c4ccccc4-c4ccccc4)c4ccccc34)c2)cc1]

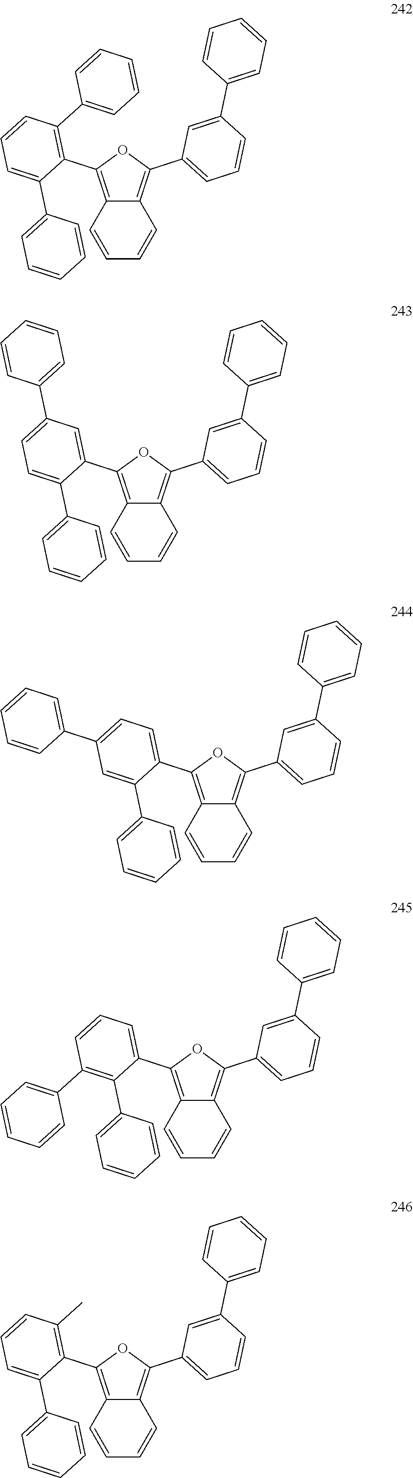 [SMILES: c1ccc(-c2cccc(-c3oc(-c4c(-c5ccccc5)cccc4-c4ccccc4)c4ccccc34)c2)cc1 ; c1ccc(-c2cccc(-c3oc(-c4cc(-c5ccccc5)ccc4-c4ccccc4)c4ccccc34)c2)cc1 ; c1ccc(-c2cccc(-c3oc(-c4ccc(-c5ccccc5)cc4-c4ccccc4)c4ccccc34)c2)cc1 ; c1ccc(-c2cccc(-c3oc(-c4cccc(-c5ccccc5)c4-c4ccccc4)c4ccccc34)c2)cc1 ; Cc1cccc(-c2ccccc2)c1-c1oc(-c2cccc(-c3ccccc3)c2)c2ccccc12]

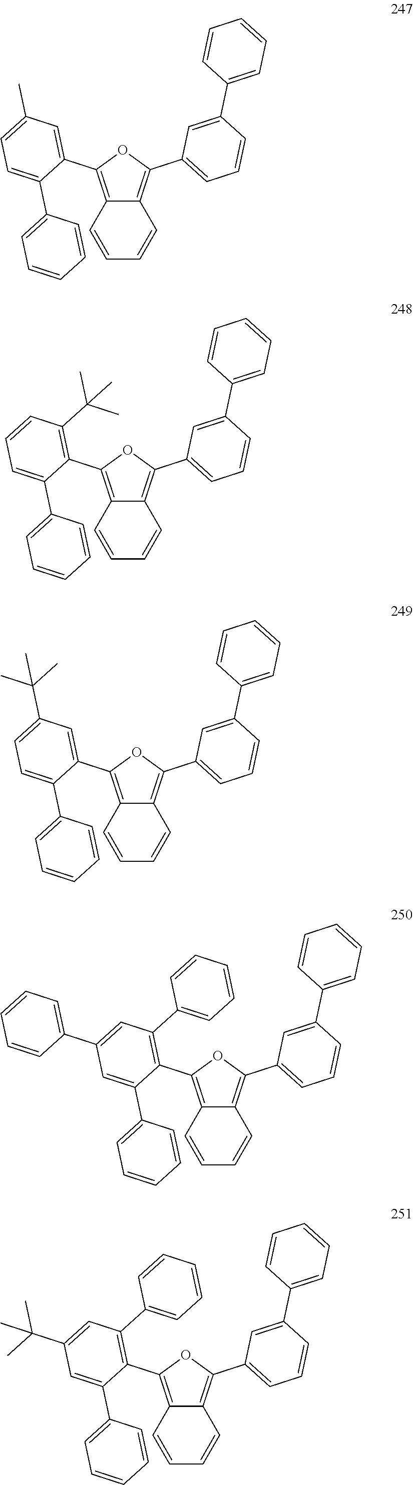 [SMILES: Cc1ccc(-c2ccccc2)c(-c2oc(-c3cccc(-c4ccccc4)c3)c3ccccc23)c1 ; CC(C)(C)c1cccc(-c2ccccc2)c1-c1oc(-c2cccc(-c3ccccc3)c2)c2ccccc12 ; CC(C)(C)c1ccc(-c2ccccc2)c(-c2oc(-c3cccc(-c4ccccc4)c3)c3ccccc23)c1 ; c1ccc(-c2cccc(-c3oc(-c4c(-c5ccccc5)cc(-c5ccccc5)cc4-c4ccccc4)c4ccccc34)c2)cc1 ; CC(C)(C)c1cc(-c2ccccc2)c(-c2oc(-c3cccc(-c4ccccc4)c3)c3ccccc23)c(-c2ccccc2)c1]

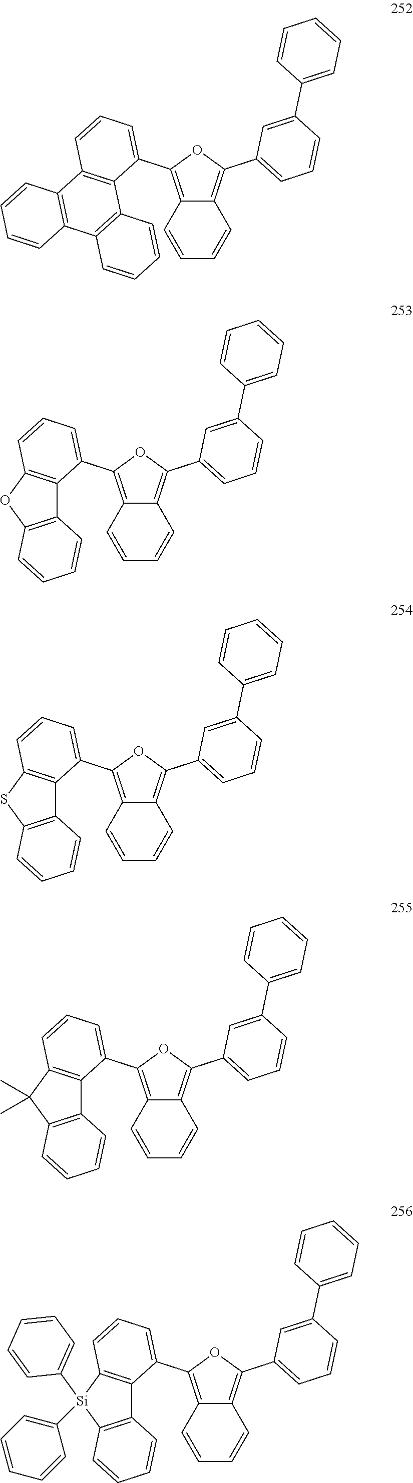 [SMILES: c1ccc(-c2cccc(-c3oc(-c4cccc5c6ccccc6c6ccccc6c45)c4ccccc34)c2)cc1 ; c1ccc(-c2cccc(-c3oc(-c4cccc5oc6ccccc6c45)c4ccccc34)c2)cc1 ; c1ccc(-c2cccc(-c3oc(-c4cccc5sc6ccccc6c45)c4ccccc34)c2)cc1 ; CC1(C)c2ccccc2-c2c(-c3oc(-c4cccc(-c5ccccc5)c4)c4ccccc34)cccc21 ; c1ccc(-c2cccc(-c3oc(-c4cccc5c4-c4ccccc4[Si]5(c4ccccc4)c4ccccc4)c4ccccc34)c2)cc1]

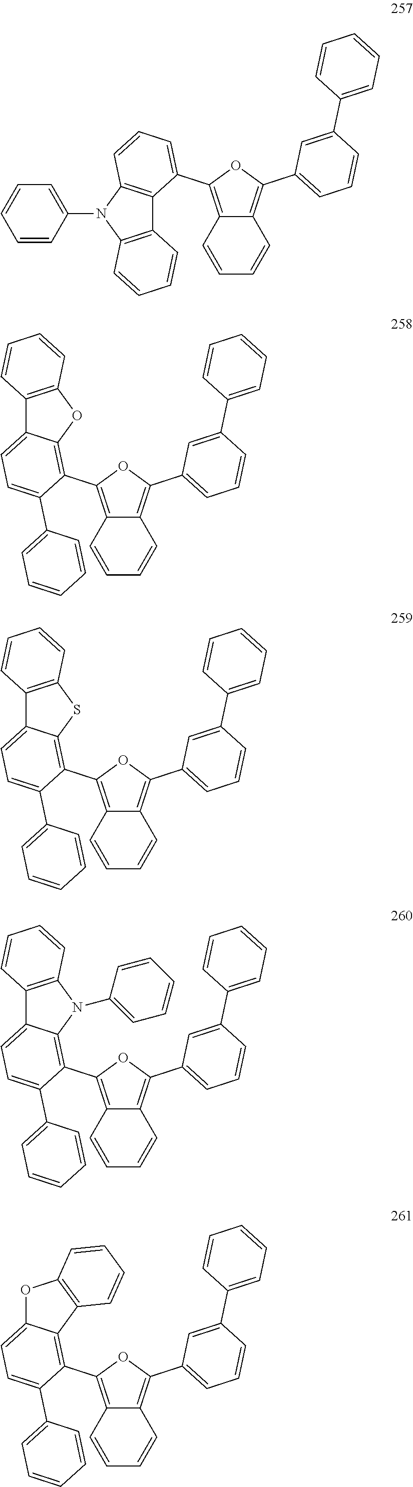

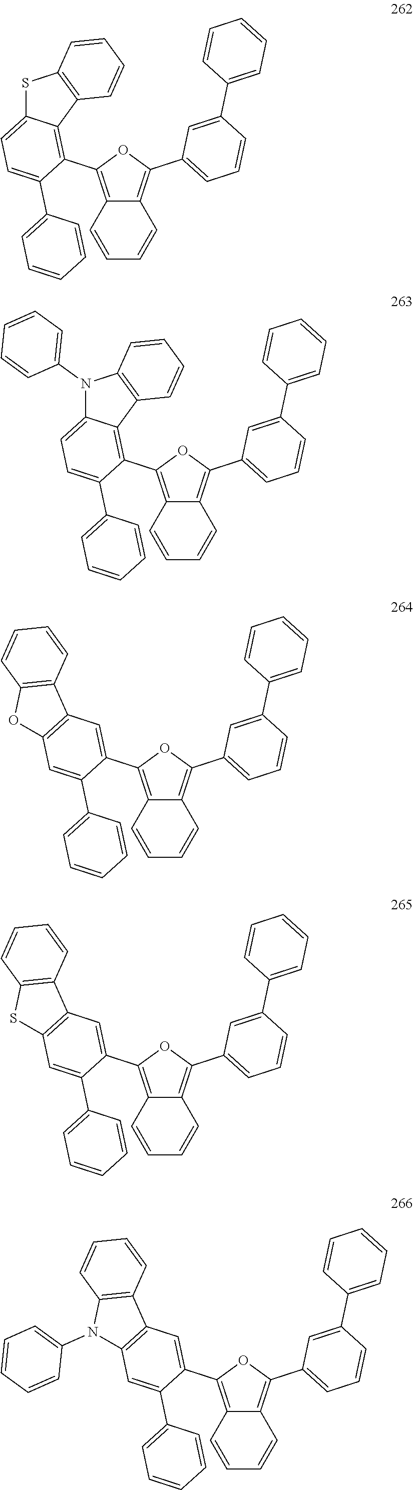

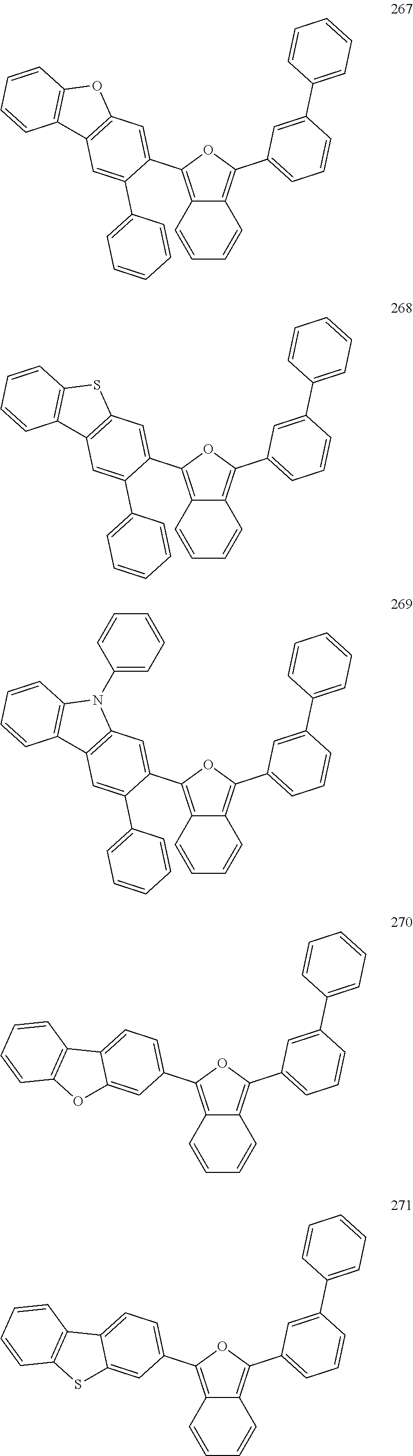

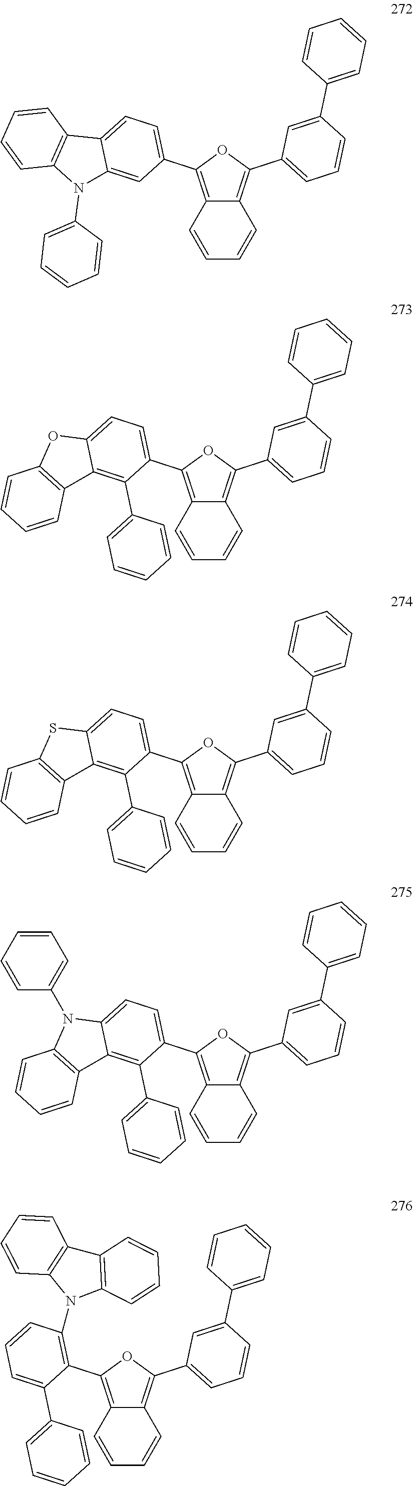 [SMILES: c1ccc(-c2cccc(-c3oc(-c4ccc5c6ccccc6n(-c6ccccc6)c5c4)c4ccccc34)c2)cc1 ; c1ccc(-c2cccc(-c3oc(-c4ccc5oc6ccccc6c5c4-c4ccccc4)c4ccccc34)c2)cc1 ; c1ccc(-c2cccc(-c3oc(-c4ccc5sc6ccccc6c5c4-c4ccccc4)c4ccccc34)c2)cc1 ; c1ccc(-c2cccc(-c3oc(-c4ccc5c(c4-c4ccccc4)c4ccccc4n5-c4ccccc4)c4ccccc34)c2)cc1 ; c1ccc(-c2cccc(-c3oc(-c4c(-c5ccccc5)cccc4-n4c5ccccc5c5ccccc54)c4ccccc34)c2)cc1]

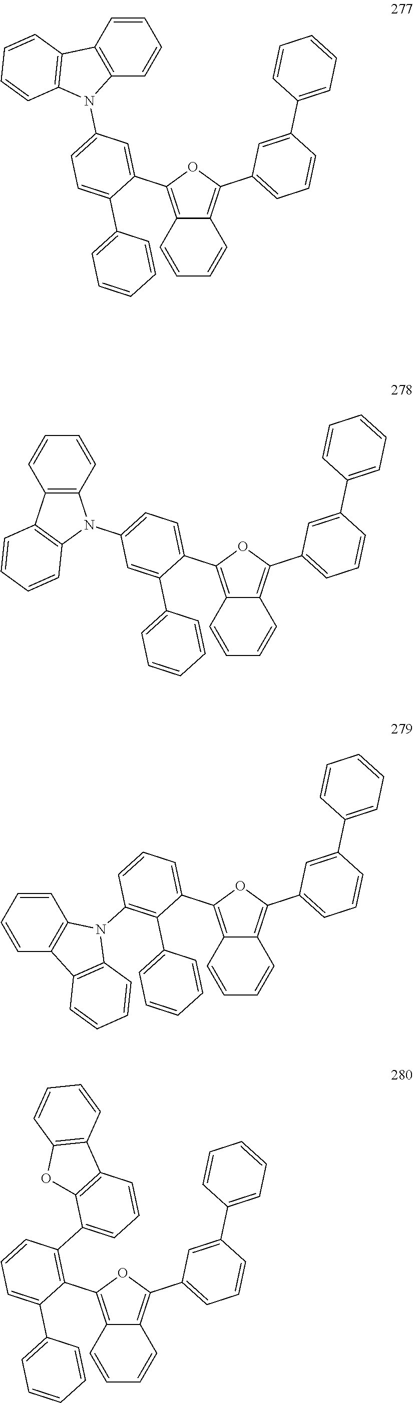 [SMILES: c1ccc(-c2cccc(-c3oc(-c4cc(-n5c6ccccc6c6ccccc65)ccc4-c4ccccc4)c4ccccc34)c2)cc1 ; c1ccc(-c2cccc(-c3oc(-c4ccc(-n5c6ccccc6c6ccccc65)cc4-c4ccccc4)c4ccccc34)c2)cc1 ; c1ccc(-c2cccc(-c3oc(-c4cccc(-n5c6ccccc6c6ccccc65)c4-c4ccccc4)c4ccccc34)c2)cc1 ; c1ccc(-c2cccc(-c3oc(-c4c(-c5ccccc5)cccc4-c4cccc5c4oc4ccccc45)c4ccccc34)c2)cc1]

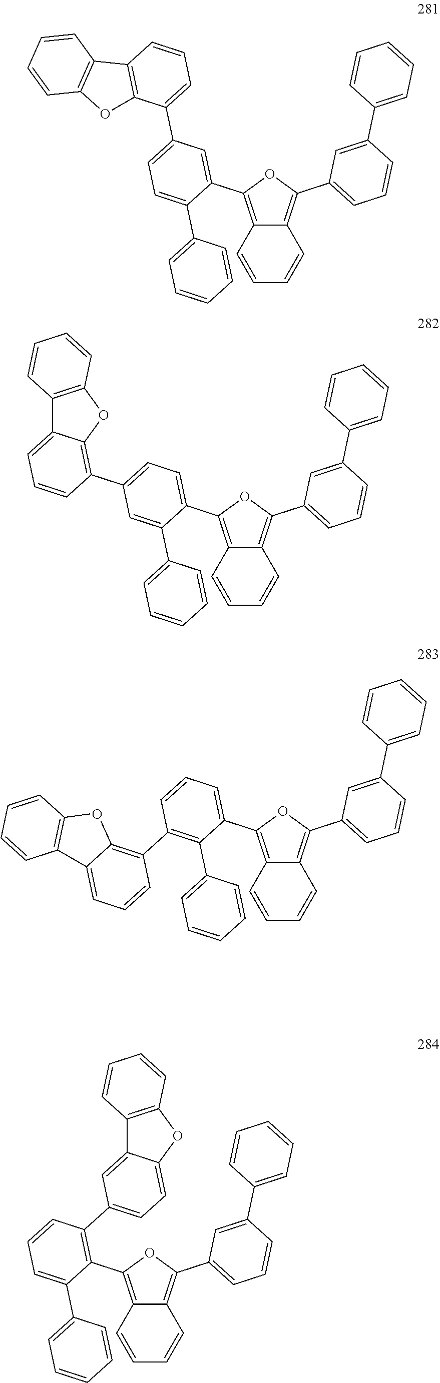 [SMILES: c1ccc(-c2cccc(-c3oc(-c4cc(-c5cccc6c5oc5ccccc56)ccc4-c4ccccc4)c4ccccc34)c2)cc1 ; c1ccc(-c2cccc(-c3oc(-c4ccc(-c5cccc6c5oc5ccccc56)cc4-c4ccccc4)c4ccccc34)c2)cc1 ; c1ccc(-c2cccc(-c3oc(-c4cccc(-c5cccc6c5oc5ccccc56)c4-c4ccccc4)c4ccccc34)c2)cc1 ; c1ccc(-c2cccc(-c3oc(-c4c(-c5ccccc5)cccc4-c4ccc5oc6ccccc6c5c4)c4ccccc34)c2)cc1]

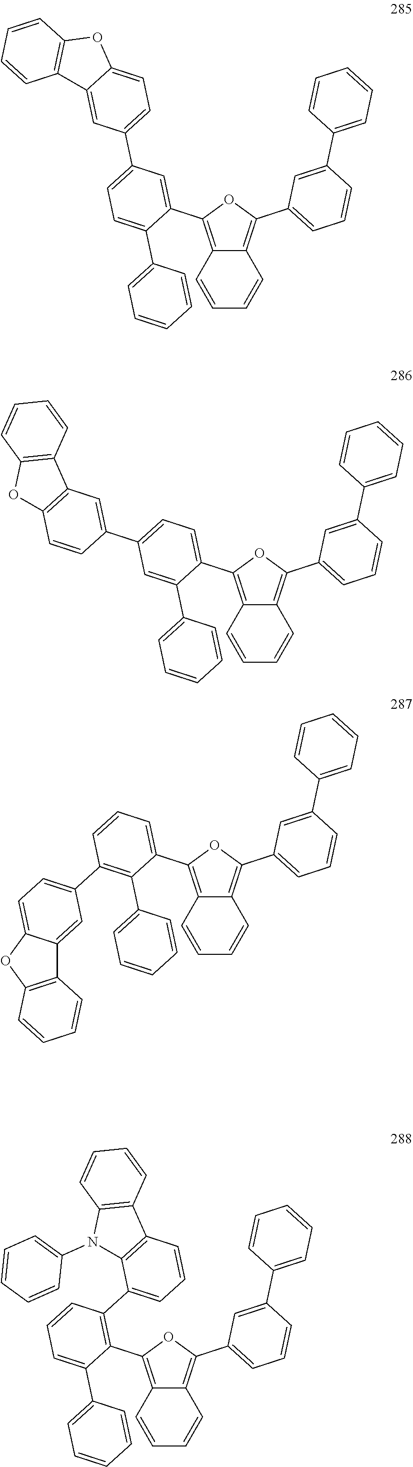 [SMILES: c1ccc(-c2cccc(-c3oc(-c4cc(-c5ccc6oc7ccccc7c6c5)ccc4-c4ccccc4)c4ccccc34)c2)cc1 ; c1ccc(-c2cccc(-c3oc(-c4ccc(-c5ccc6oc7ccccc7c6c5)cc4-c4ccccc4)c4ccccc34)c2)cc1 ; c1ccc(-c2cccc(-c3oc(-c4cccc(-c5ccc6oc7ccccc7c6c5)c4-c4ccccc4)c4ccccc34)c2)cc1 ; c1ccc(-c2cccc(-c3oc(-c4c(-c5ccccc5)cccc4-c4cccc5c6ccccc6n(-c6ccccc6)c45)c4ccccc34)c2)cc1]

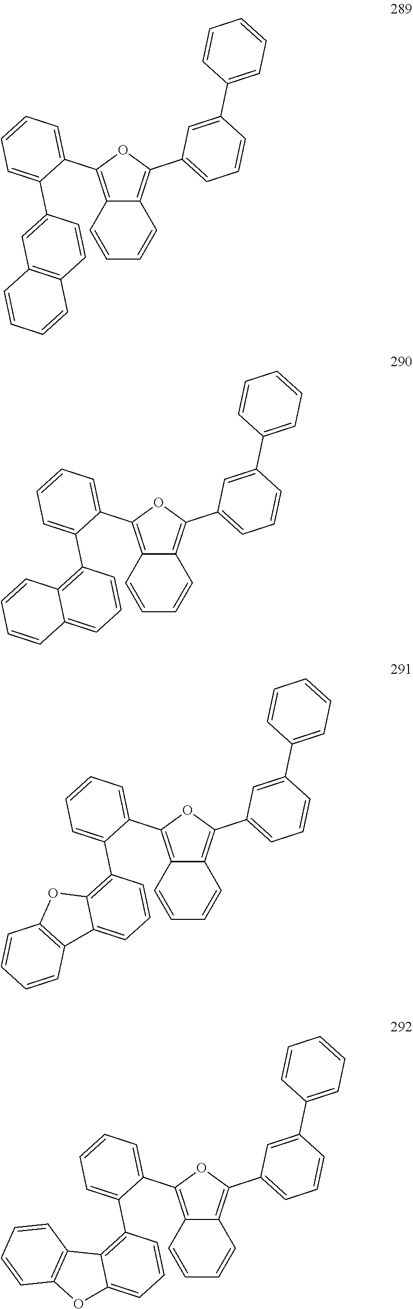 [SMILES: c1ccc(-c2cccc(-c3oc(-c4ccccc4-c4ccc5ccccc5c4)c4ccccc34)c2)cc1 ; c1ccc(-c2cccc(-c3oc(-c4ccccc4-c4cccc5ccccc45)c4ccccc34)c2)cc1 ; c1ccc(-c2cccc(-c3oc(-c4ccccc4-c4cccc5c4oc4ccccc45)c4ccccc34)c2)cc1 ; c1ccc(-c2cccc(-c3oc(-c4ccccc4-c4cccc5oc6ccccc6c45)c4ccccc34)c2)cc1]

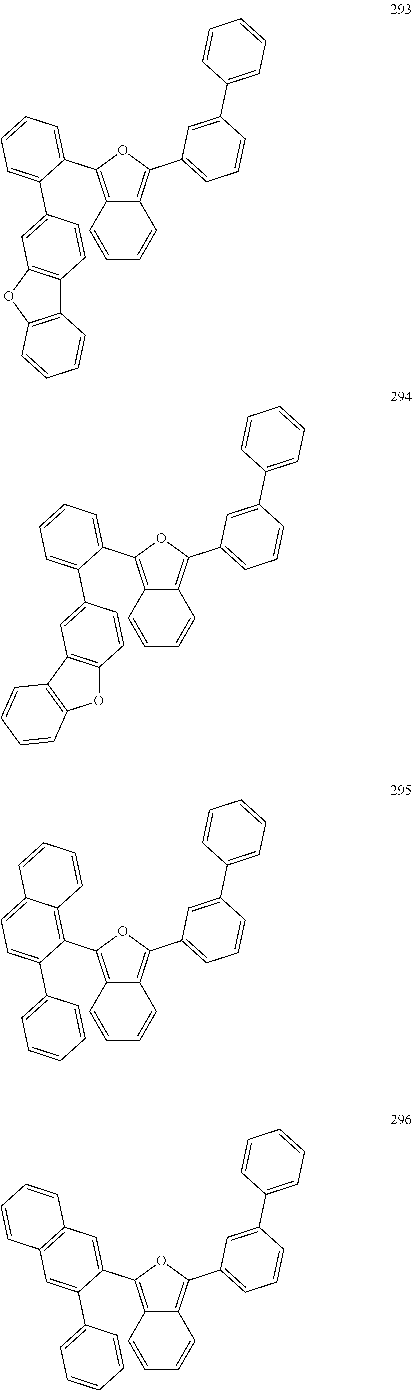 [SMILES: c1ccc(-c2cccc(-c3oc(-c4ccccc4-c4ccc5c(c4)oc4ccccc45)c4ccccc34)c2)cc1 ; c1ccc(-c2cccc(-c3oc(-c4ccccc4-c4ccc5oc6ccccc6c5c4)c4ccccc34)c2)cc1 ; c1ccc(-c2cccc(-c3oc(-c4c(-c5ccccc5)ccc5ccccc45)c4ccccc34)c2)cc1 ; c1ccc(-c2cccc(-c3oc(-c4cc5ccccc5cc4-c4ccccc4)c4ccccc34)c2)cc1]

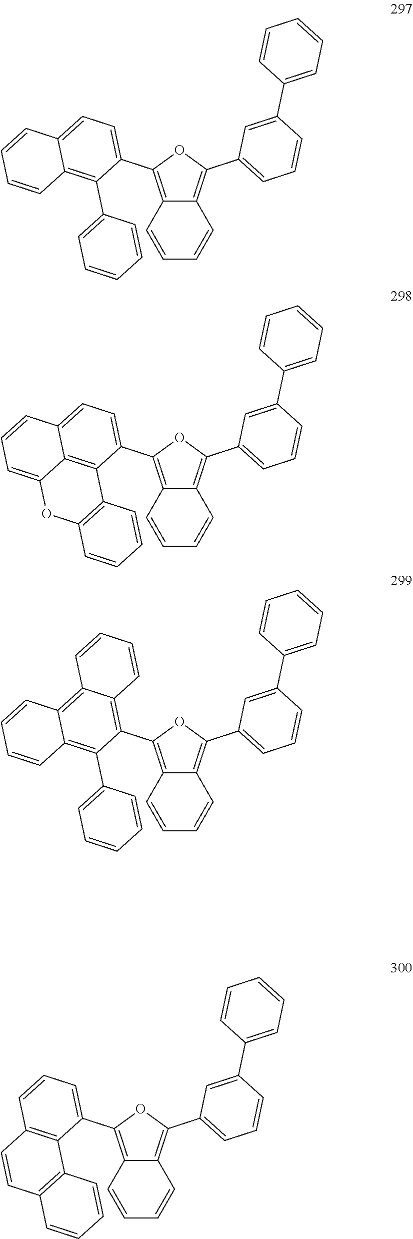 [SMILES: c1ccc(-c2cccc(-c3oc(-c4ccc5ccccc5c4-c4ccccc4)c4ccccc34)c2)cc1 ; c1ccc(-c2cccc(-c3oc(-c4ccc5cccc6c5c4-c4ccccc4O6)c4ccccc34)c2)cc1 ; c1ccc(-c2cccc(-c3oc(-c4c(-c5ccccc5)c5ccccc5c5ccccc45)c4ccccc34)c2)cc1 ; c1ccc(-c2cccc(-c3oc(-c4cccc5ccc6ccccc6c45)c4ccccc34)c2)cc1]

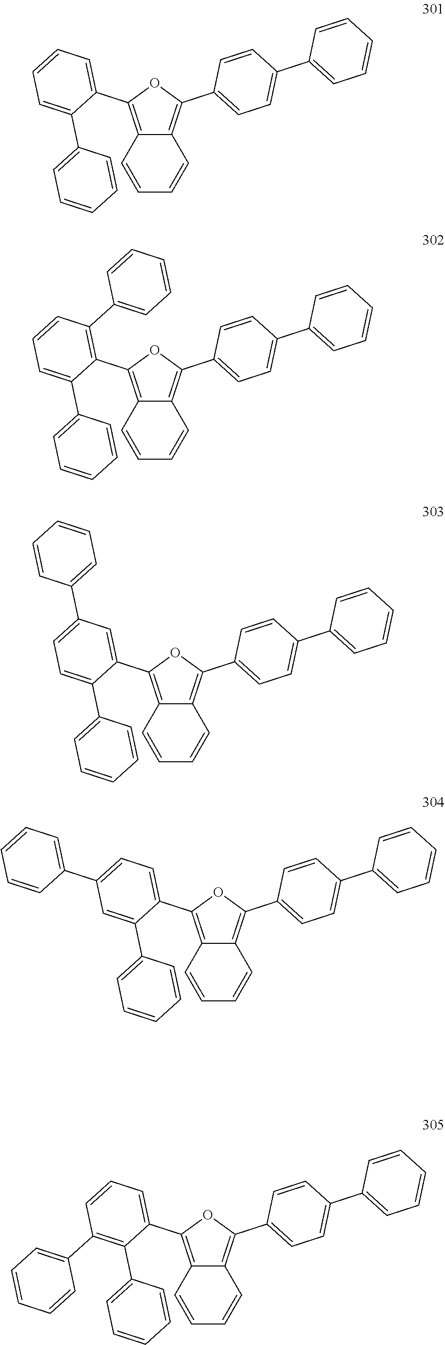 [SMILES: c1ccc(-c2ccc(-c3oc(-c4ccccc4-c4ccccc4)c4ccccc34)cc2)cc1 ; c1ccc(-c2ccc(-c3oc(-c4c(-c5ccccc5)cccc4-c4ccccc4)c4ccccc34)cc2)cc1 ; c1ccc(-c2ccc(-c3oc(-c4cc(-c5ccccc5)ccc4-c4ccccc4)c4ccccc34)cc2)cc1 ; c1ccc(-c2ccc(-c3oc(-c4ccc(-c5ccccc5)cc4-c4ccccc4)c4ccccc34)cc2)cc1 ; c1ccc(-c2ccc(-c3oc(-c4cccc(-c5ccccc5)c4-c4ccccc4)c4ccccc34)cc2)cc1]

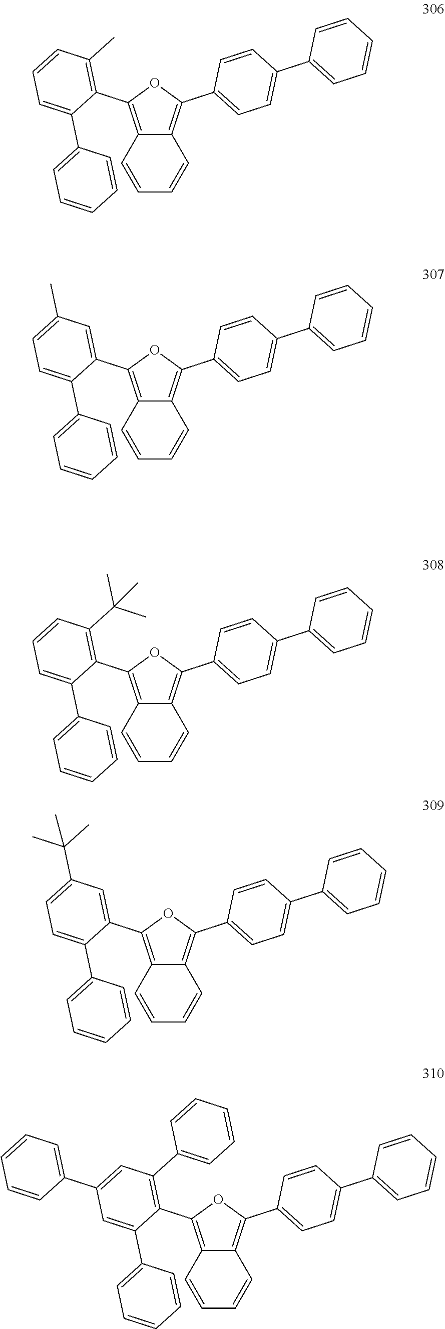

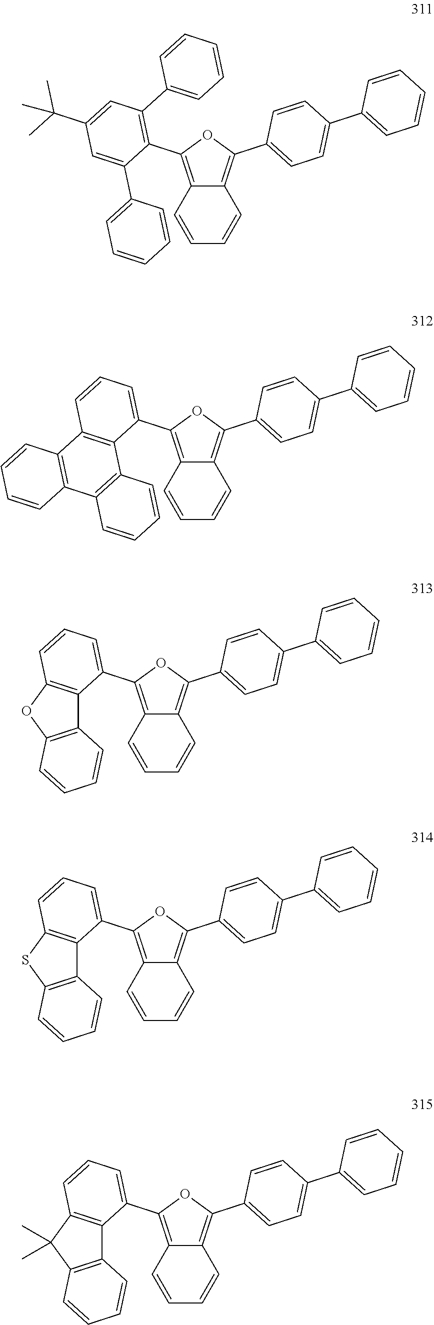 [SMILES: CC(C)(C)c1cc(-c2ccccc2)c(-c2oc(-c3ccc(-c4ccccc4)cc3)c3ccccc23)c(-c2ccccc2)c1 ; c1ccc(-c2ccc(-c3oc(-c4cccc5c6ccccc6c6ccccc6c45)c4ccccc34)cc2)cc1 ; c1ccc(-c2ccc(-c3oc(-c4cccc5oc6ccccc6c45)c4ccccc34)cc2)cc1 ; c1ccc(-c2ccc(-c3oc(-c4cccc5sc6ccccc6c45)c4ccccc34)cc2)cc1 ; CC1(C)c2ccccc2-c2c(-c3oc(-c4ccc(-c5ccccc5)cc4)c4ccccc34)cccc21]

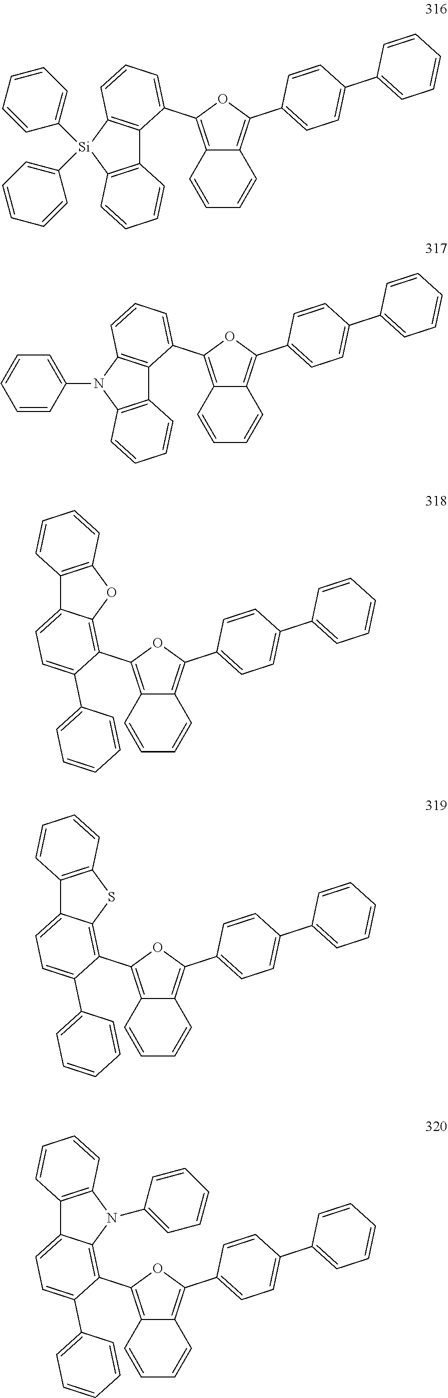 [SMILES: c1ccc(-c2ccc(-c3oc(-c4cccc5c4-c4ccccc4[Si]5(c4ccccc4)c4ccccc4)c4ccccc34)cc2)cc1 ; c1ccc(-c2ccc(-c3oc(-c4cccc5c4c4ccccc4n5-c4ccccc4)c4ccccc34)cc2)cc1 ; c1ccc(-c2ccc(-c3oc(-c4c(-c5ccccc5)ccc5c4oc4ccccc45)c4ccccc34)cc2)cc1 ; c1ccc(-c2ccc(-c3oc(-c4c(-c5ccccc5)ccc5c4sc4ccccc45)c4ccccc34)cc2)cc1 ; c1ccc(-c2ccc(-c3oc(-c4c(-c5ccccc5)ccc5c6ccccc6n(-c6ccccc6)c45)c4ccccc34)cc2)cc1]

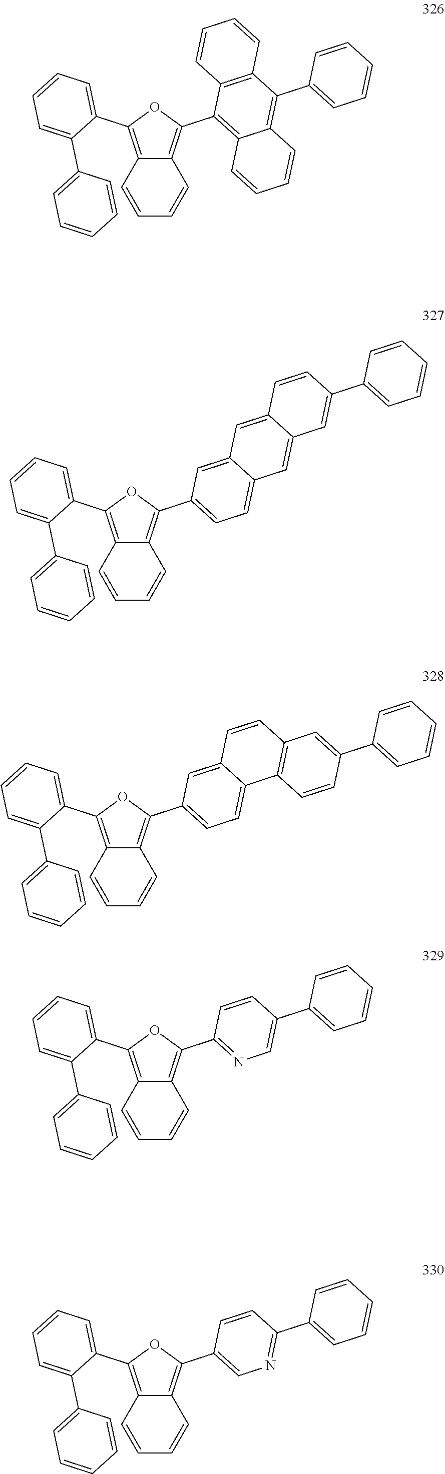 [SMILES: c1ccc(-c2ccccc2-c2oc(-c3c4ccccc4c(-c4ccccc4)c4ccccc34)c3ccccc23)cc1 ; c1ccc(-c2ccc3cc4cc(-c5oc(-c6ccccc6-c6ccccc6)c6ccccc56)ccc4cc3c2)cc1 ; c1ccc(-c2ccc3c(ccc4cc(-c5oc(-c6ccccc6-c6ccccc6)c6ccccc56)ccc43)c2)cc1 ; c1ccc(-c2ccc(-c3oc(-c4ccccc4-c4ccccc4)c4ccccc34)nc2)cc1 ; c1ccc(-c2ccc(-c3oc(-c4ccccc4-c4ccccc4)c4ccccc34)cn2)cc1]

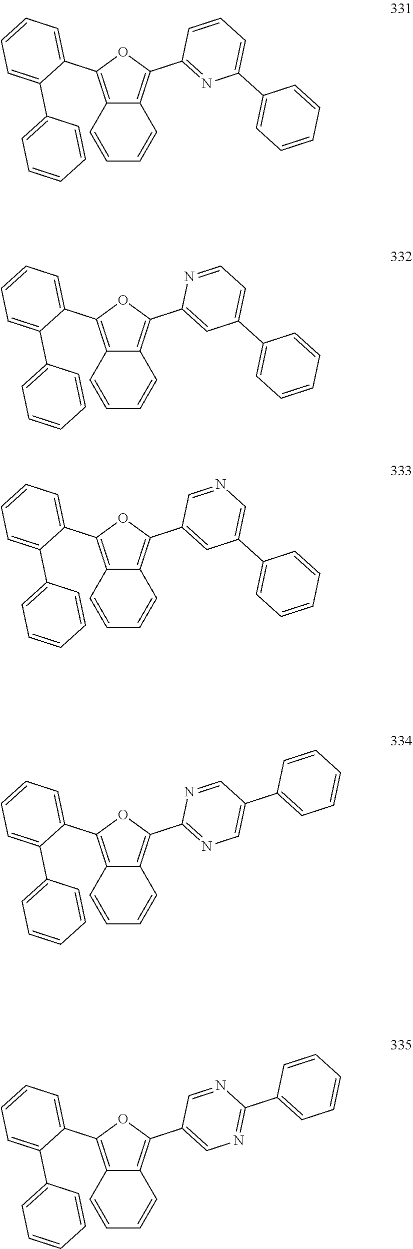 [SMILES: c1ccc(-c2cccc(-c3oc(-c4ccccc4-c4ccccc4)c4ccccc34)n2)cc1 ; c1ccc(-c2ccnc(-c3oc(-c4ccccc4-c4ccccc4)c4ccccc34)c2)cc1 ; c1ccc(-c2cncc(-c3oc(-c4ccccc4-c4ccccc4)c4ccccc34)c2)cc1 ; c1ccc(-c2cnc(-c3oc(-c4ccccc4-c4ccccc4)c4ccccc34)nc2)cc1 ; c1ccc(-c2ncc(-c3oc(-c4ccccc4-c4ccccc4)c4ccccc34)cn2)cc1]

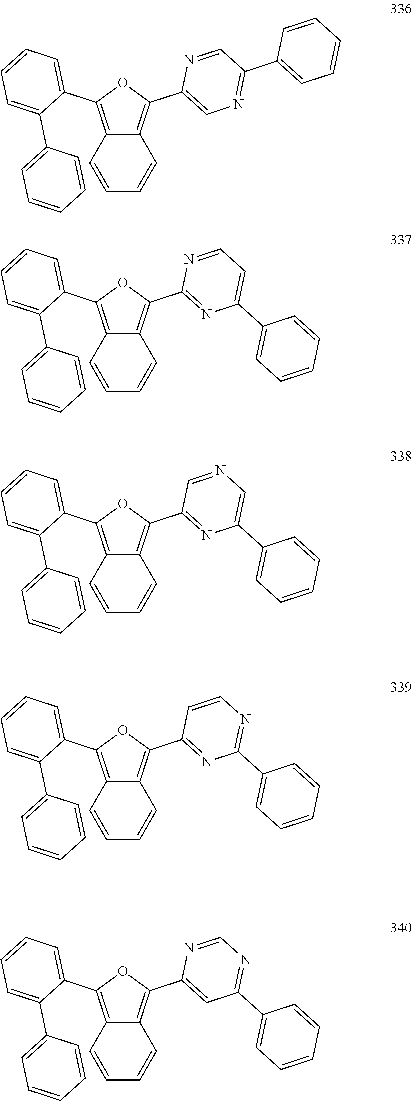 [SMILES: c1ccc(-c2cnc(-c3oc(-c4ccccc4-c4ccccc4)c4ccccc34)cn2)cc1 ; c1ccc(-c2ccnc(-c3oc(-c4ccccc4-c4ccccc4)c4ccccc34)n2)cc1 ; c1ccc(-c2cncc(-c3oc(-c4ccccc4-c4ccccc4)c4ccccc34)n2)cc1 ; c1ccc(-c2nccc(-c3oc(-c4ccccc4-c4ccccc4)c4ccccc34)n2)cc1 ; c1ccc(-c2cc(-c3oc(-c4ccccc4-c4ccccc4)c4ccccc34)ncn2)cc1]

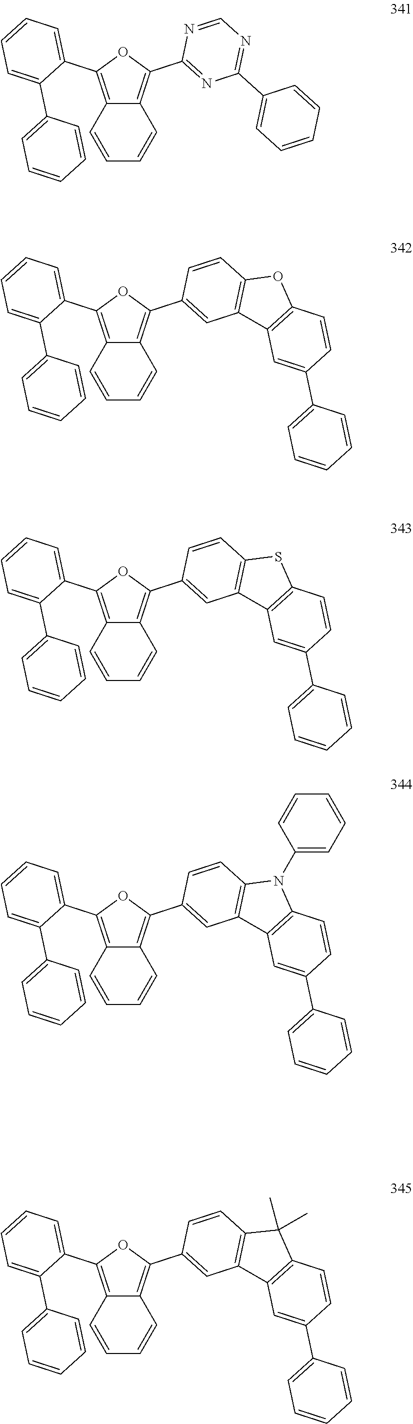

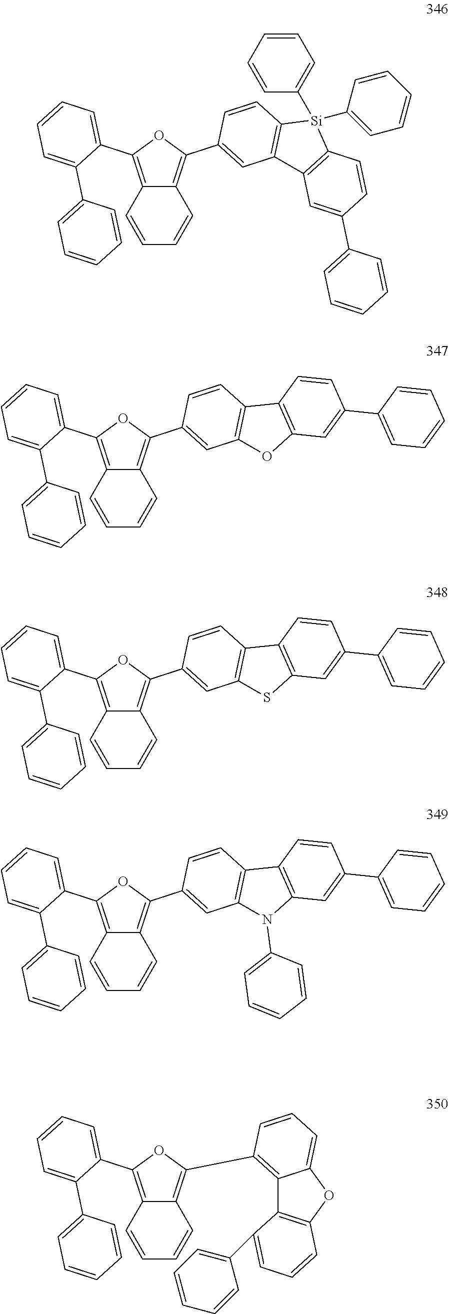 [SMILES: c1ccc(-c2ccc3c(c2)-c2cc(-c4oc(-c5ccccc5-c5ccccc5)c5ccccc45)ccc2[Si]3(c2ccccc2)c2ccccc2)cc1 ; c1ccc(-c2ccc3c(c2)oc2cc(-c4oc(-c5ccccc5-c5ccccc5)c5ccccc45)ccc23)cc1 ; c1ccc(-c2ccc3c(c2)sc2cc(-c4oc(-c5ccccc5-c5ccccc5)c5ccccc45)ccc23)cc1 ; c1ccc(-c2ccc3c4ccc(-c5oc(-c6ccccc6-c6ccccc6)c6ccccc56)cc4n(-c4ccccc4)c3c2)cc1 ; c1ccc(-c2ccccc2-c2oc(-c3cccc4oc5cccc(-c6ccccc6)c5c34)c3ccccc23)cc1]

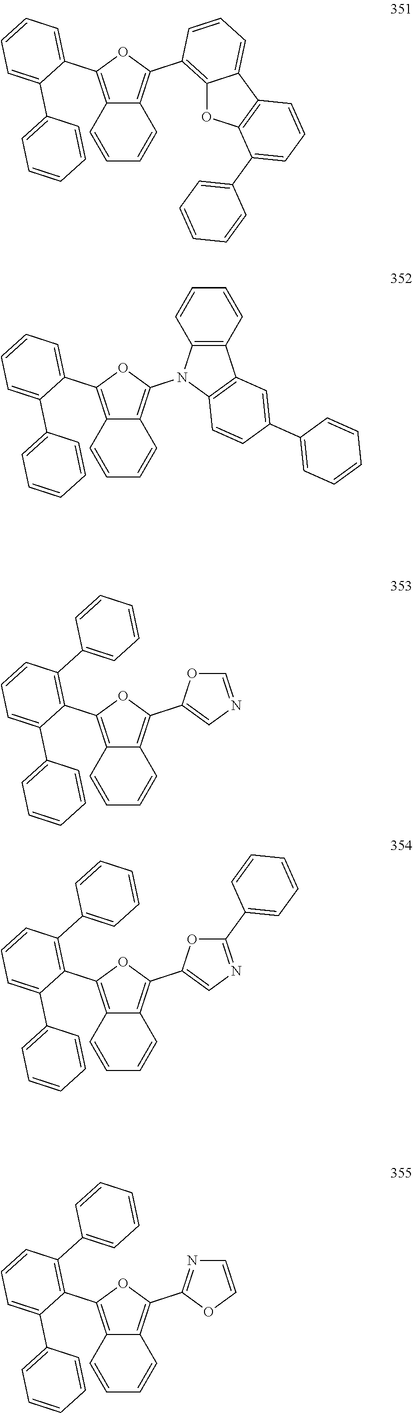

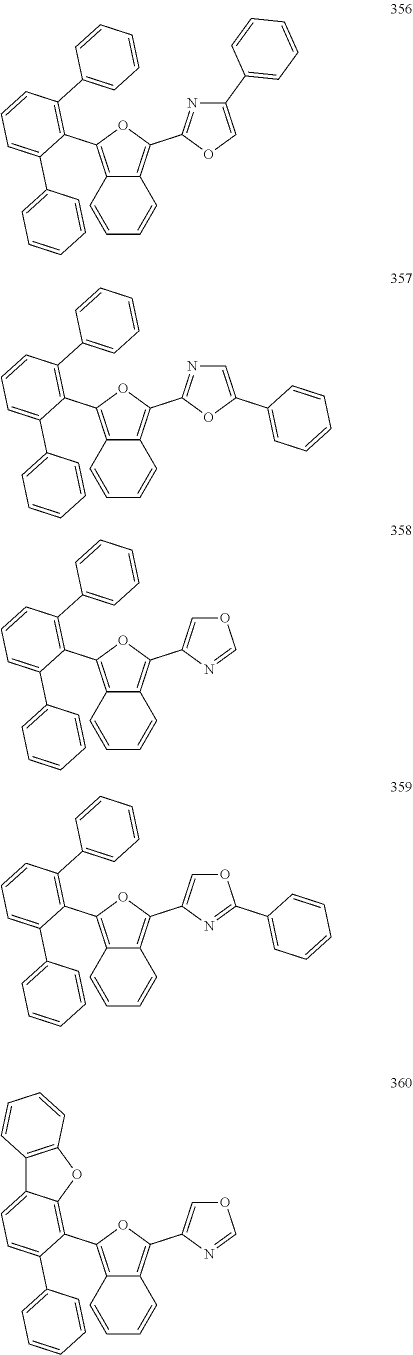

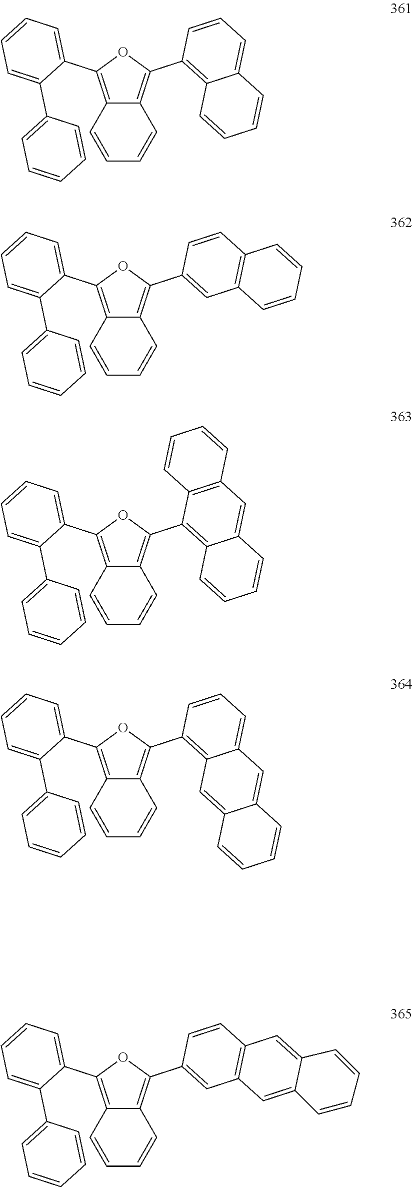 [SMILES: c1ccc(-c2ccccc2-c2oc(-c3cccc4ccccc34)c3ccccc23)cc1 ; c1ccc(-c2ccccc2-c2oc(-c3ccc4ccccc4c3)c3ccccc23)cc1 ; c1ccc(-c2ccccc2-c2oc(-c3c4ccccc4cc4ccccc34)c3ccccc23)cc1 ; c1ccc(-c2ccccc2-c2oc(-c3cccc4cc5ccccc5cc34)c3ccccc23)cc1 ; c1ccc(-c2ccccc2-c2oc(-c3ccc4cc5ccccc5cc4c3)c3ccccc23)cc1]

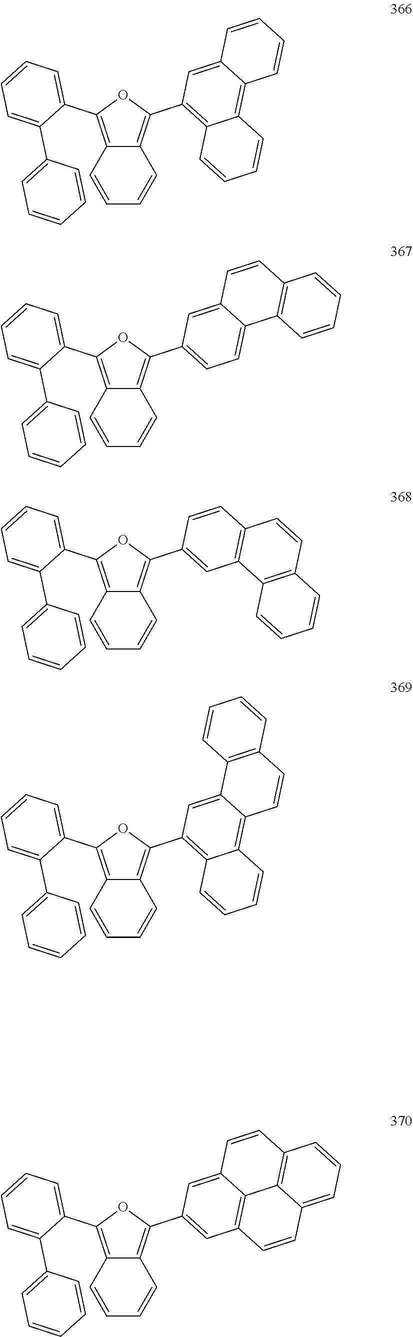 [SMILES: c1ccc(-c2ccccc2-c2oc(-c3cc4ccccc4c4ccccc34)c3ccccc23)cc1 ; c1ccc(-c2ccccc2-c2oc(-c3ccc4c(ccc5ccccc54)c3)c3ccccc23)cc1 ; c1ccc(-c2ccccc2-c2oc(-c3ccc4ccc5ccccc5c4c3)c3ccccc23)cc1 ; c1ccc(-c2ccccc2-c2oc(-c3cc4c5ccccc5ccc4c4ccccc34)c3ccccc23)cc1 ; c1ccc(-c2ccccc2-c2oc(-c3cc4ccc5cccc6ccc(c3)c4c56)c3ccccc23)cc1]

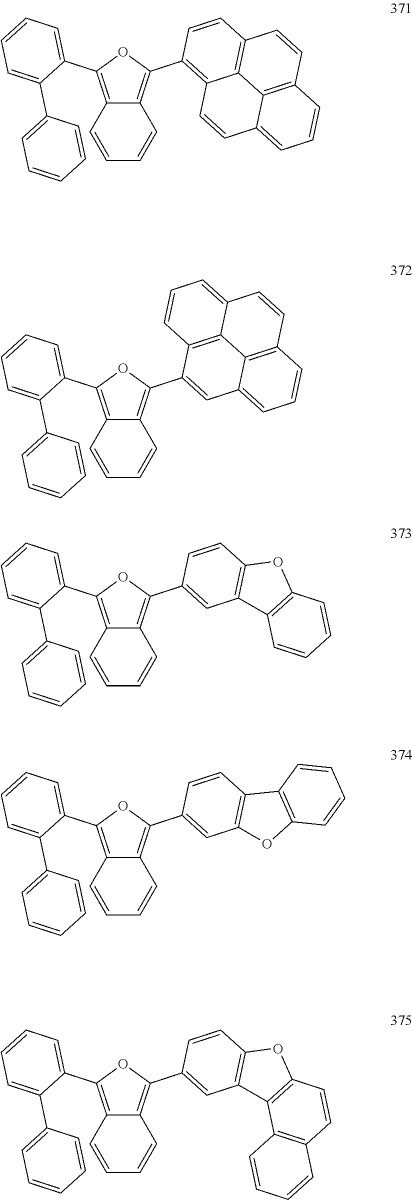 [SMILES: c1ccc(-c2ccccc2-c2oc(-c3ccc4ccc5cccc6ccc3c4c56)c3ccccc23)cc1 ; c1ccc(-c2ccccc2-c2oc(-c3cc4cccc5ccc6cccc3c6c54)c3ccccc23)cc1 ; c1ccc(-c2ccccc2-c2oc(-c3ccc4oc5ccccc5c4c3)c3ccccc23)cc1 ; c1ccc(-c2ccccc2-c2oc(-c3ccc4c(c3)oc3ccccc34)c3ccccc23)cc1 ; c1ccc(-c2ccccc2-c2oc(-c3ccc4oc5ccc6ccccc6c5c4c3)c3ccccc23)cc1]

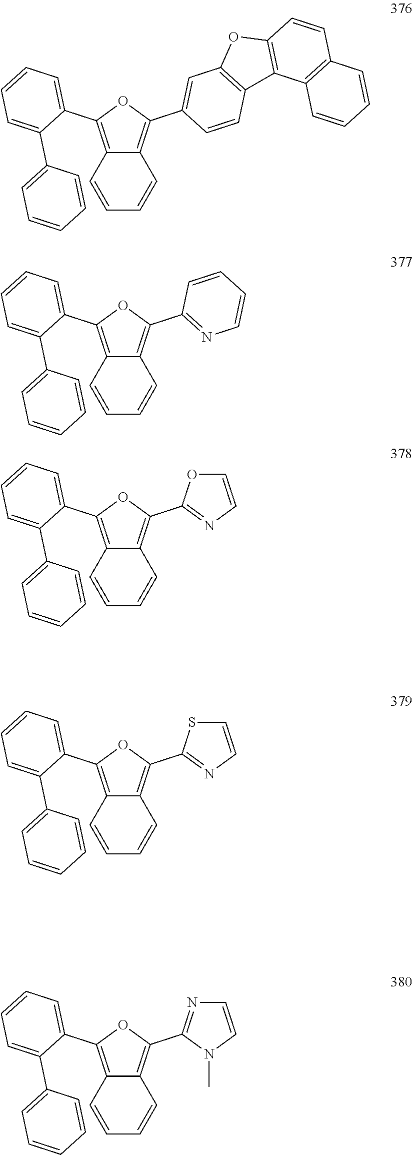

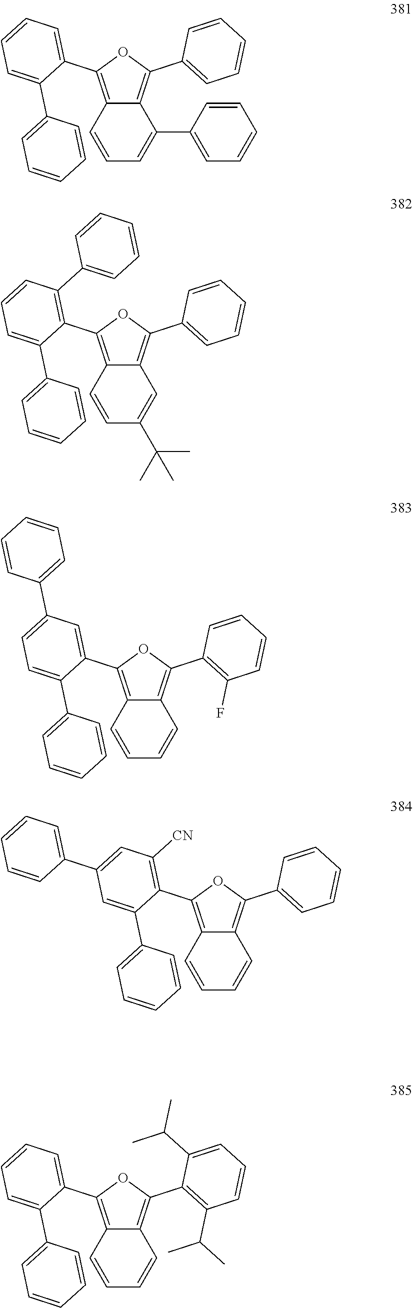 [SMILES: c1ccc(-c2ccccc2-c2oc(-c3ccccc3)c3c(-c4ccccc4)cccc23)cc1 ; CC(C)(C)c1ccc2c(-c3c(-c4ccccc4)cccc3-c3ccccc3)oc(-c3ccccc3)c2c1 ; Fc1ccccc1-c1oc(-c2cc(-c3ccccc3)ccc2-c2ccccc2)c2ccccc12 ; N#Cc1cc(-c2ccccc2)cc(-c2ccccc2)c1-c1oc(-c2ccccc2)c2ccccc12 ; CC(C)c1cccc(C(C)C)c1-c1oc(-c2ccccc2-c2ccccc2)c2ccccc12]

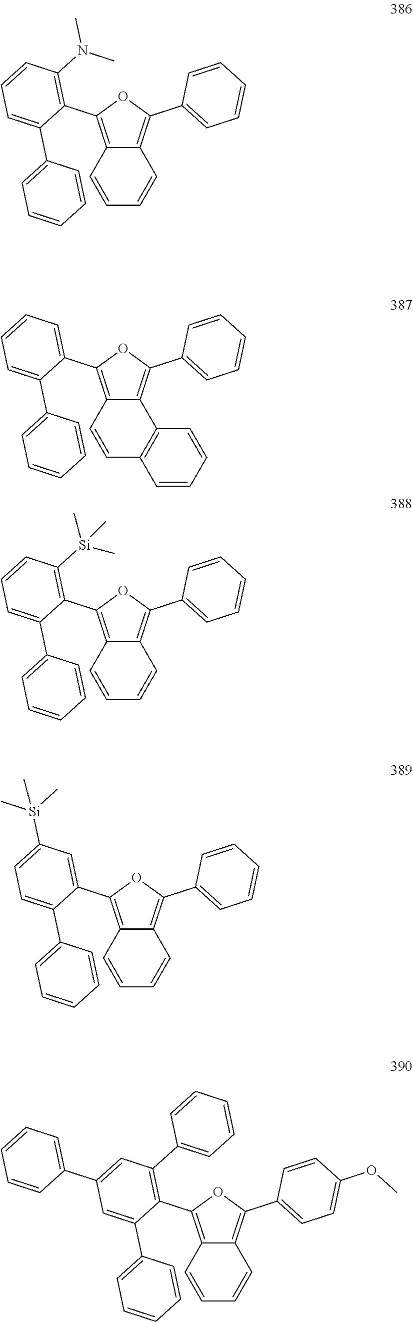

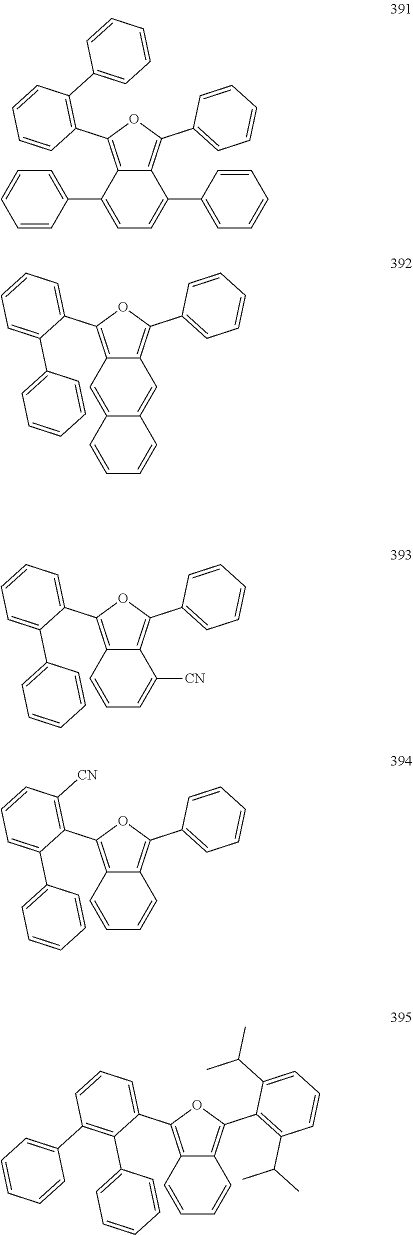

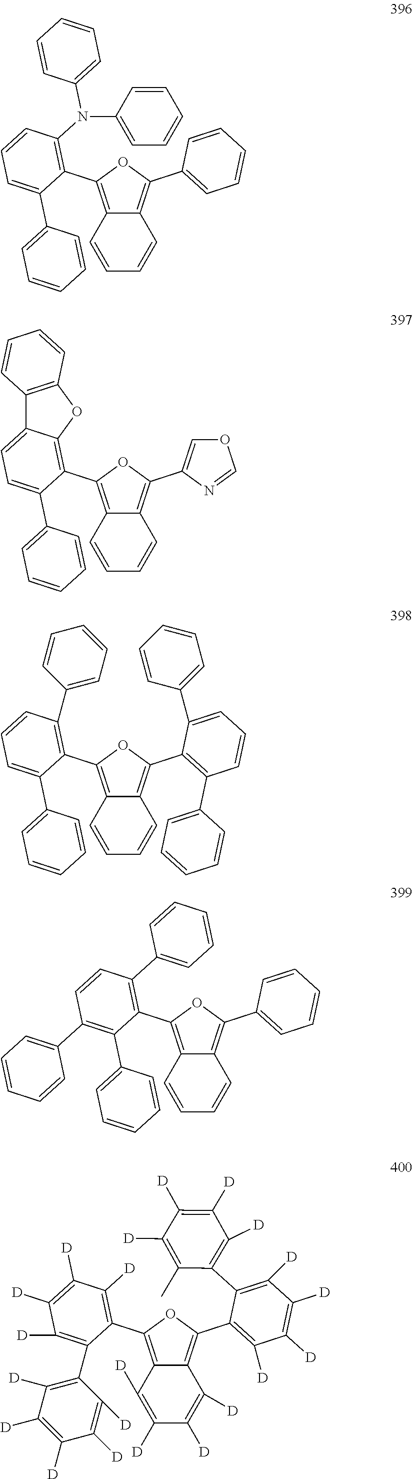

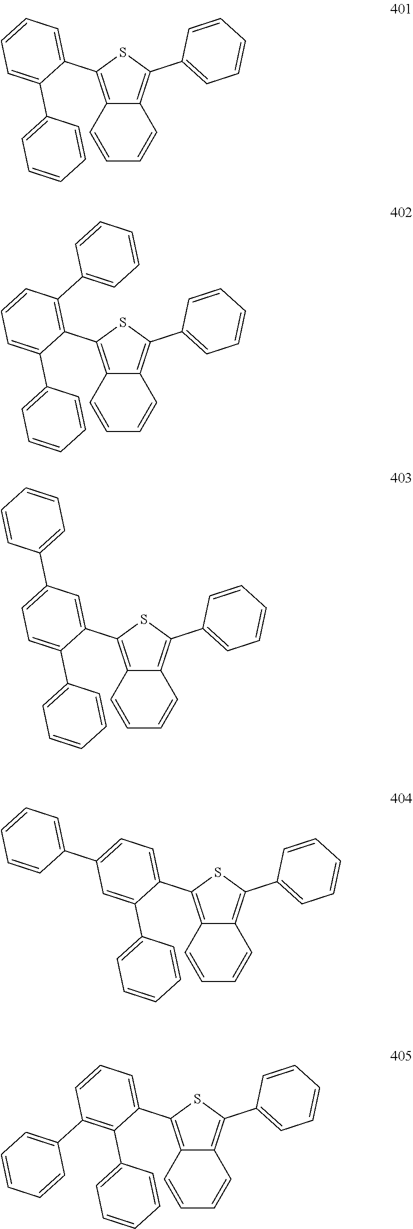

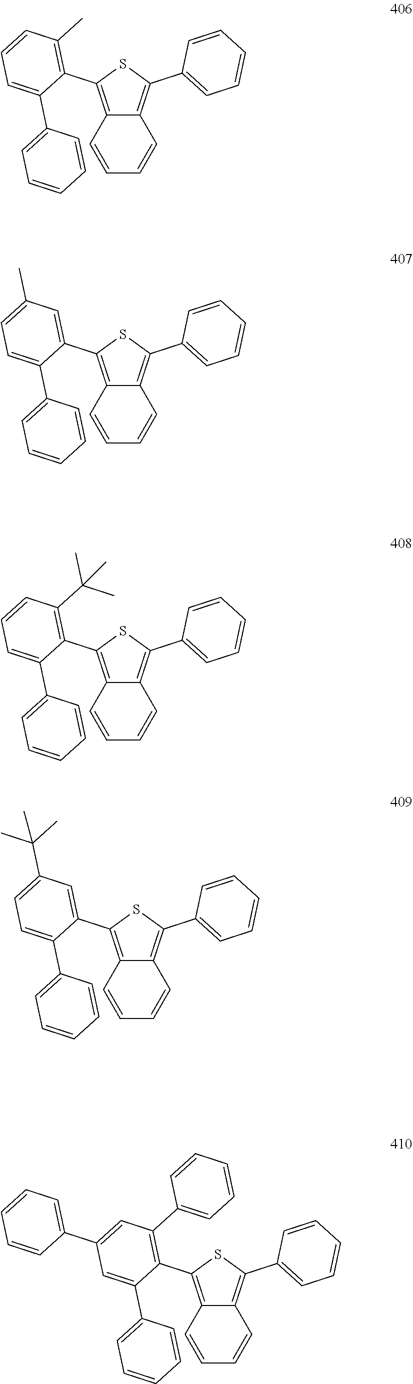 [SMILES: Cc1cccc(-c2ccccc2)c1-c1sc(-c2ccccc2)c2ccccc12 ; Cc1ccc(-c2ccccc2)c(-c2sc(-c3ccccc3)c3ccccc23)c1 ; CC(C)(C)c1cccc(-c2ccccc2)c1-c1sc(-c2ccccc2)c2ccccc12 ; CC(C)(C)c1ccc(-c2ccccc2)c(-c2sc(-c3ccccc3)c3ccccc23)c1 ; c1ccc(-c2cc(-c3ccccc3)c(-c3sc(-c4ccccc4)c4ccccc34)c(-c3ccccc3)c2)cc1]

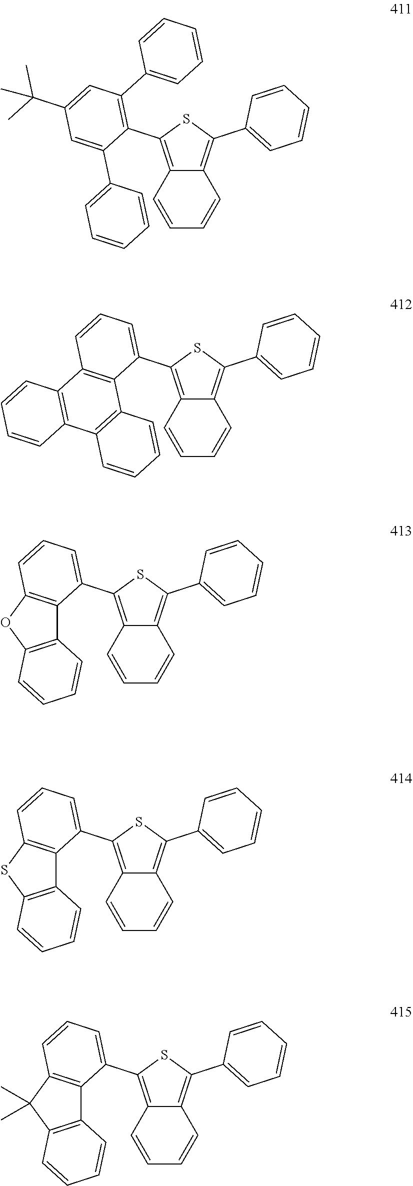 [SMILES: CC(C)(C)c1cc(-c2ccccc2)c(-c2sc(-c3ccccc3)c3ccccc23)c(-c2ccccc2)c1 ; c1ccc(-c2sc(-c3cccc4c5ccccc5c5ccccc5c34)c3ccccc23)cc1 ; c1ccc(-c2sc(-c3cccc4oc5ccccc5c34)c3ccccc23)cc1 ; c1ccc(-c2sc(-c3cccc4sc5ccccc5c34)c3ccccc23)cc1 ; CC1(C)c2ccccc2-c2c(-c3sc(-c4ccccc4)c4ccccc34)cccc21]

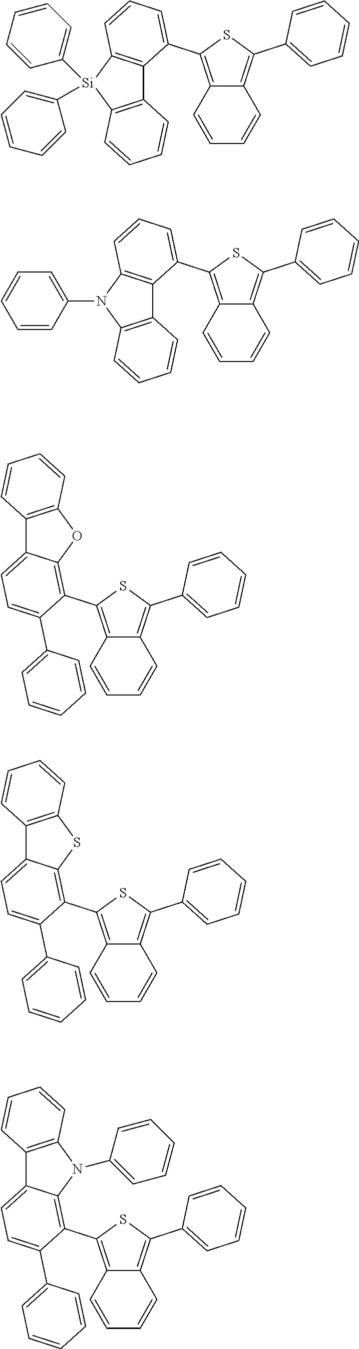 [SMILES: c1ccc(-c2sc(-c3cccc4c3-c3ccccc3[Si]4(c3ccccc3)c3ccccc3)c3ccccc23)cc1 ; c1ccc(-c2sc(-c3cccc4c3c3ccccc3n4-c3ccccc3)c3ccccc23)cc1 ; c1ccc(-c2ccc3c(oc4ccccc43)c2-c2sc(-c3ccccc3)c3ccccc23)cc1 ; c1ccc(-c2ccc3c(sc4ccccc43)c2-c2sc(-c3ccccc3)c3ccccc23)cc1 ; c1ccc(-c2ccc3c4ccccc4n(-c4ccccc4)c3c2-c2sc(-c3ccccc3)c3ccccc23)cc1]

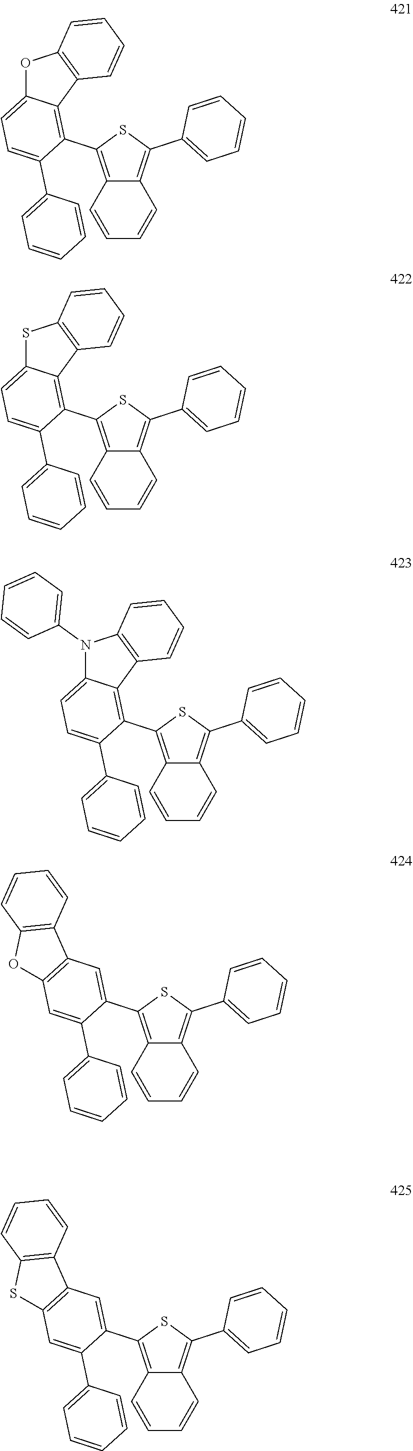

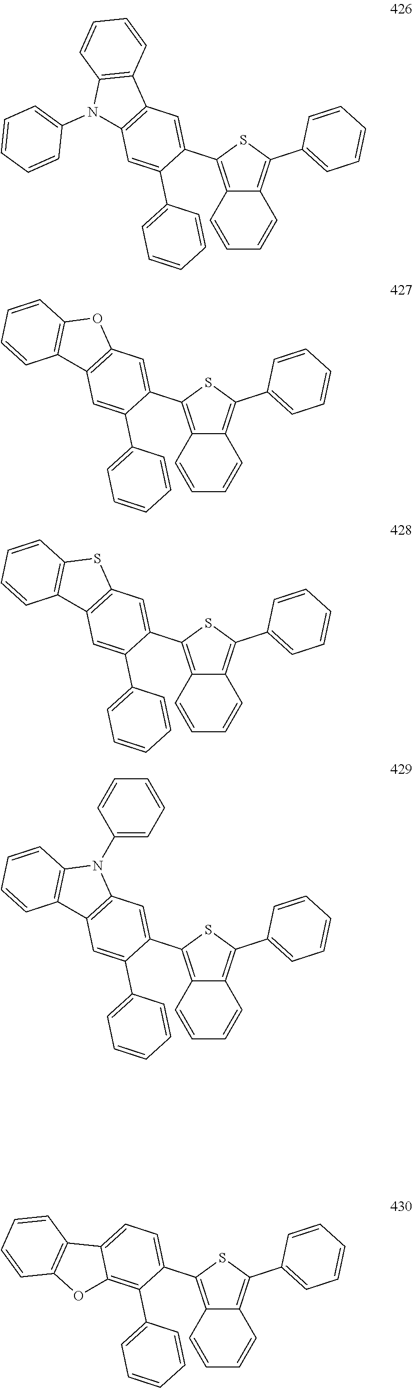

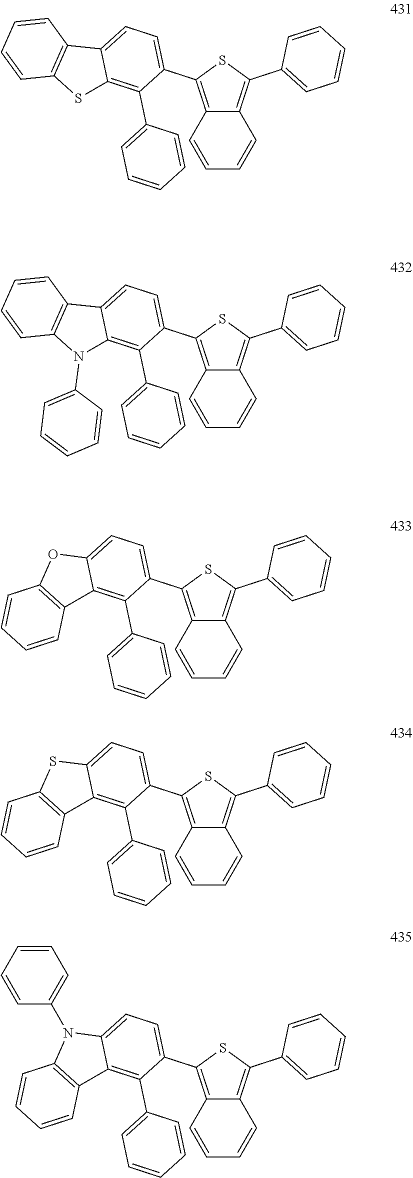 [SMILES: c1ccc(-c2sc(-c3ccc4c(sc5ccccc54)c3-c3ccccc3)c3ccccc23)cc1 ; c1ccc(-c2sc(-c3ccc4c5ccccc5n(-c5ccccc5)c4c3-c3ccccc3)c3ccccc23)cc1 ; c1ccc(-c2sc(-c3ccc4oc5ccccc5c4c3-c3ccccc3)c3ccccc23)cc1 ; c1ccc(-c2sc(-c3ccc4sc5ccccc5c4c3-c3ccccc3)c3ccccc23)cc1 ; c1ccc(-c2sc(-c3ccc4c(c3-c3ccccc3)c3ccccc3n4-c3ccccc3)c3ccccc23)cc1]

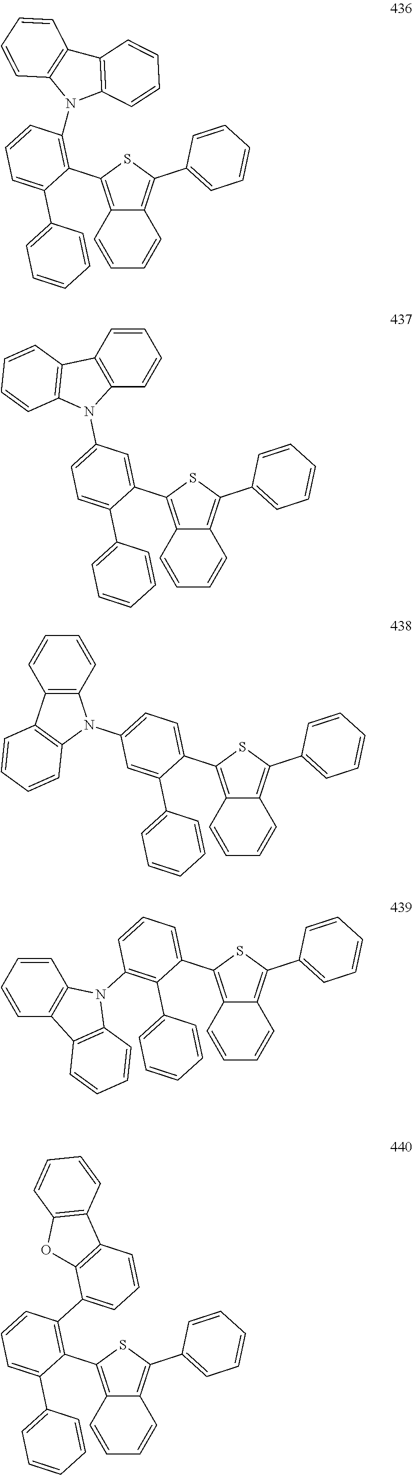

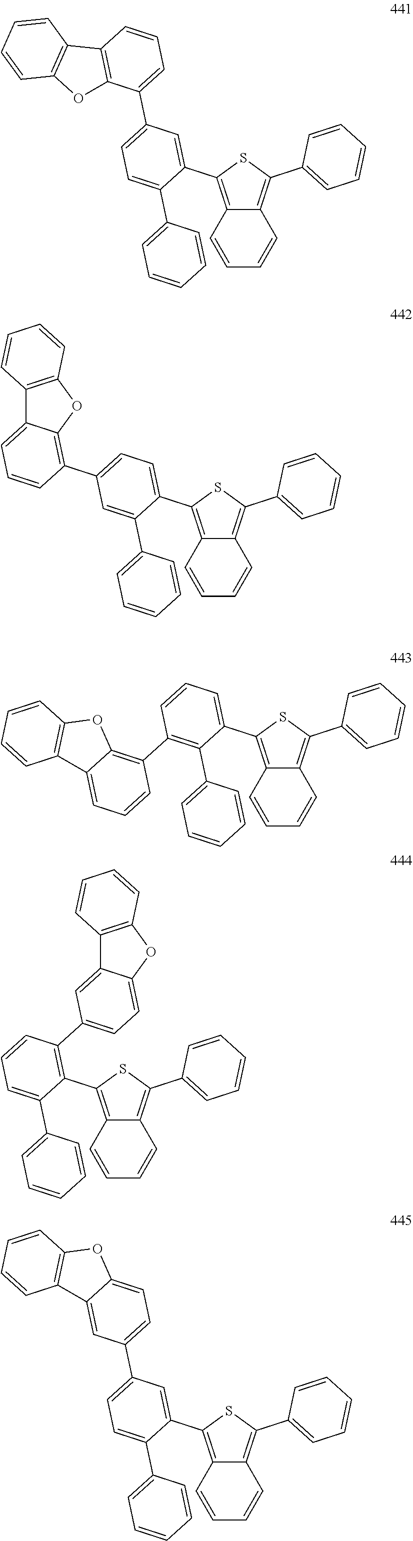

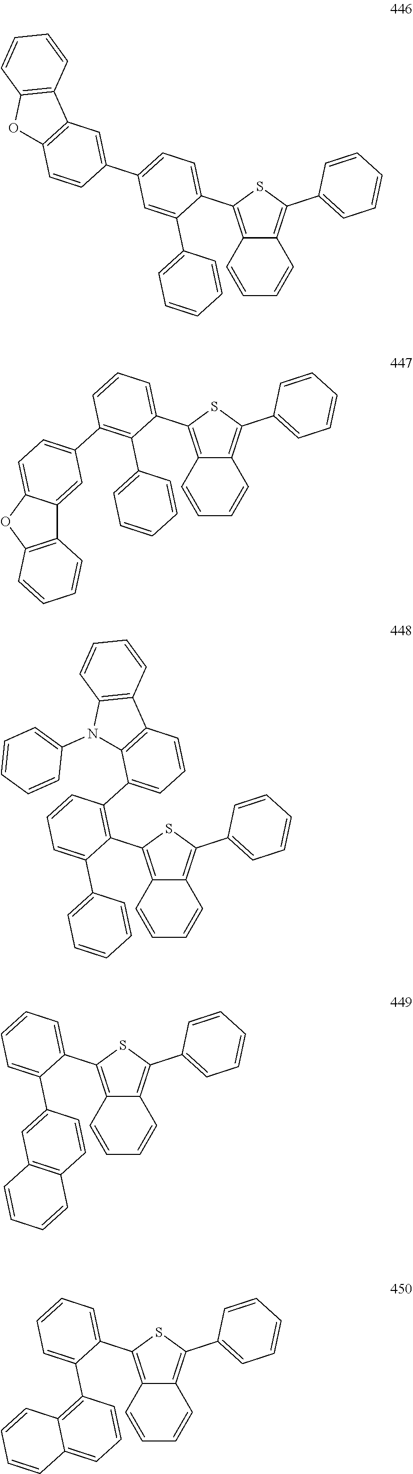 [SMILES: c1ccc(-c2cc(-c3ccc4oc5ccccc5c4c3)ccc2-c2sc(-c3ccccc3)c3ccccc23)cc1 ; c1ccc(-c2c(-c3ccc4oc5ccccc5c4c3)cccc2-c2sc(-c3ccccc3)c3ccccc23)cc1 ; c1ccc(-c2cccc(-c3cccc4c5ccccc5n(-c5ccccc5)c34)c2-c2sc(-c3ccccc3)c3ccccc23)cc1 ; c1ccc(-c2sc(-c3ccccc3-c3ccc4ccccc4c3)c3ccccc23)cc1 ; c1ccc(-c2sc(-c3ccccc3-c3cccc4ccccc34)c3ccccc23)cc1]

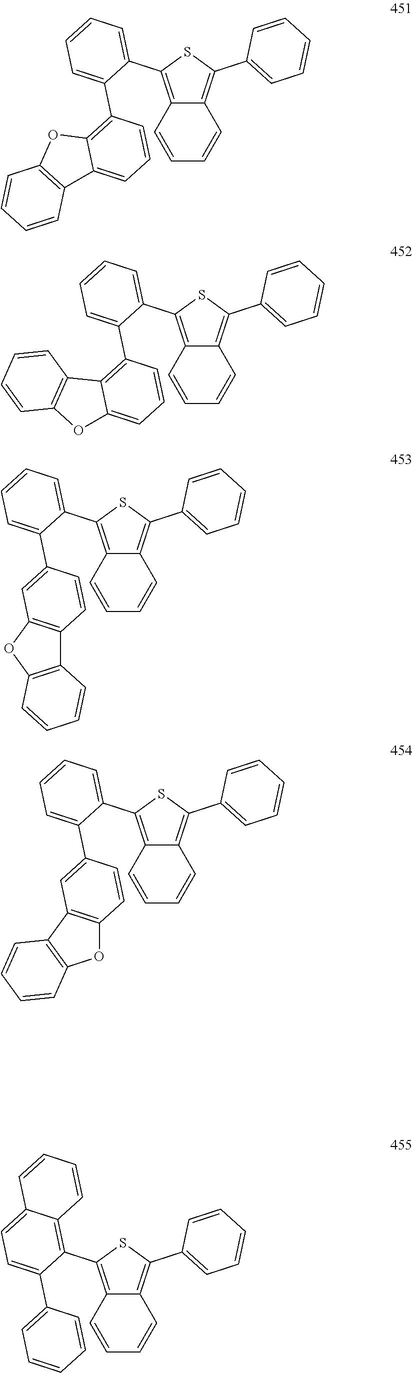

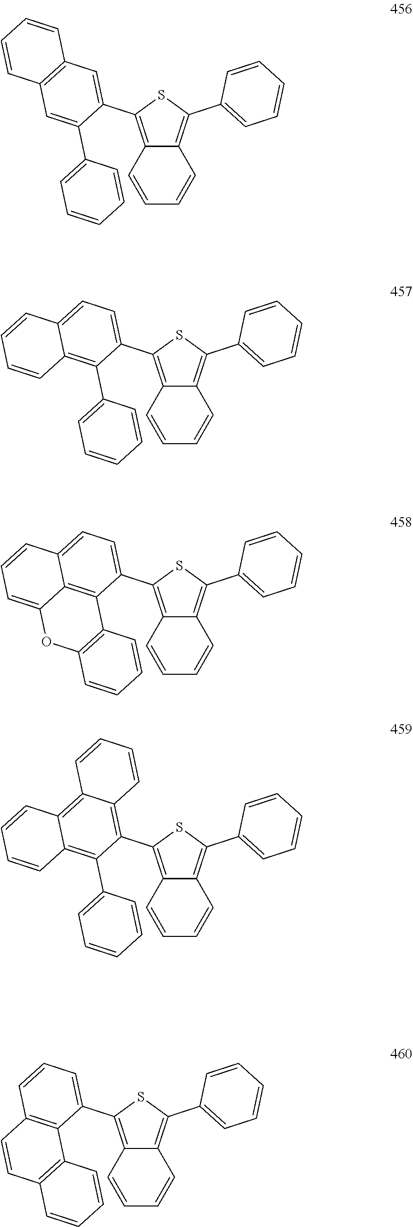 [SMILES: c1ccc(-c2cc3ccccc3cc2-c2sc(-c3ccccc3)c3ccccc23)cc1 ; c1ccc(-c2c(-c3sc(-c4ccccc4)c4ccccc34)ccc3ccccc23)cc1 ; c1ccc(-c2sc(-c3ccc4cccc5c4c3-c3ccccc3O5)c3ccccc23)cc1 ; c1ccc(-c2sc(-c3c(-c4ccccc4)c4ccccc4c4ccccc34)c3ccccc23)cc1 ; c1ccc(-c2sc(-c3cccc4ccc5ccccc5c34)c3ccccc23)cc1]

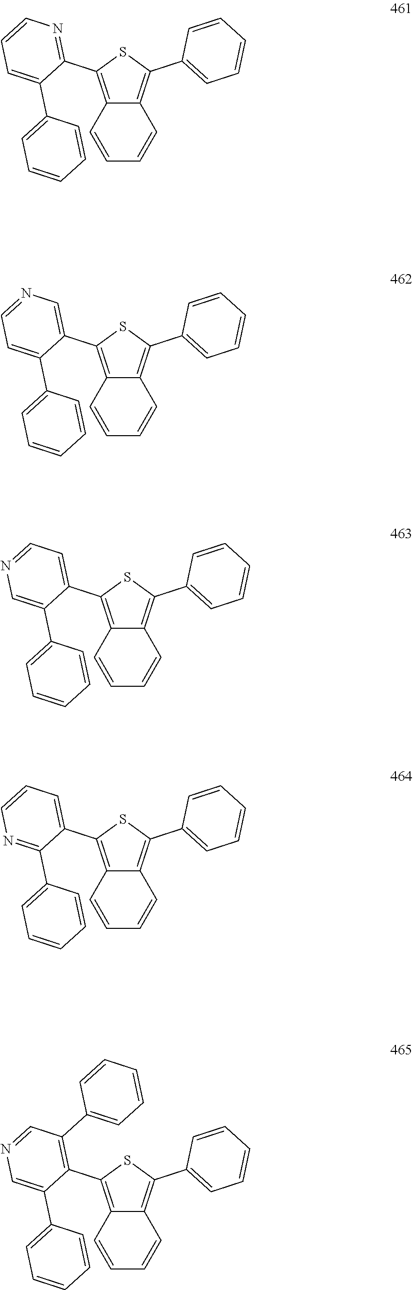 [SMILES: c1ccc(-c2cccnc2-c2sc(-c3ccccc3)c3ccccc23)cc1 ; c1ccc(-c2ccncc2-c2sc(-c3ccccc3)c3ccccc23)cc1 ; c1ccc(-c2cnccc2-c2sc(-c3ccccc3)c3ccccc23)cc1 ; c1ccc(-c2ncccc2-c2sc(-c3ccccc3)c3ccccc23)cc1 ; c1ccc(-c2cncc(-c3ccccc3)c2-c2sc(-c3ccccc3)c3ccccc23)cc1]

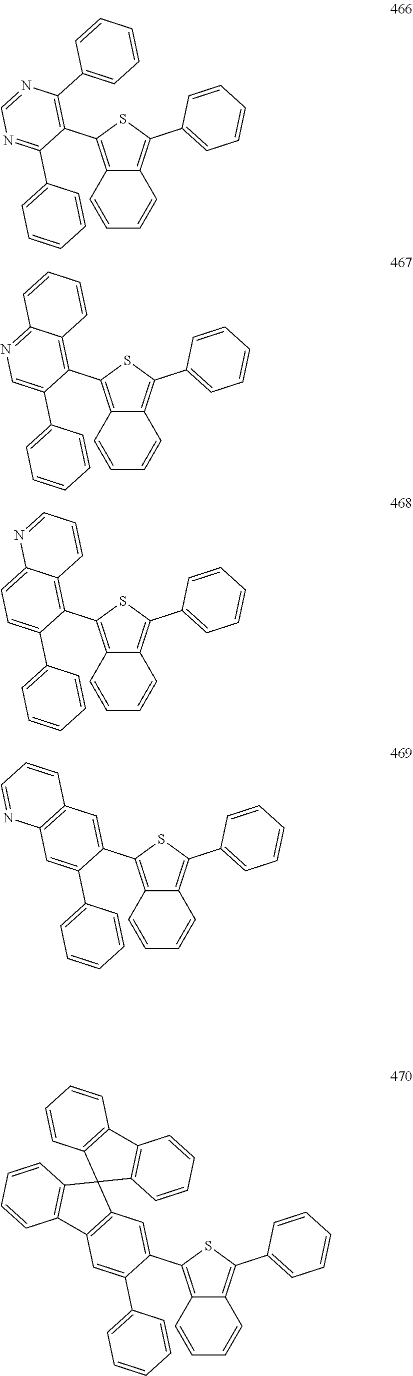

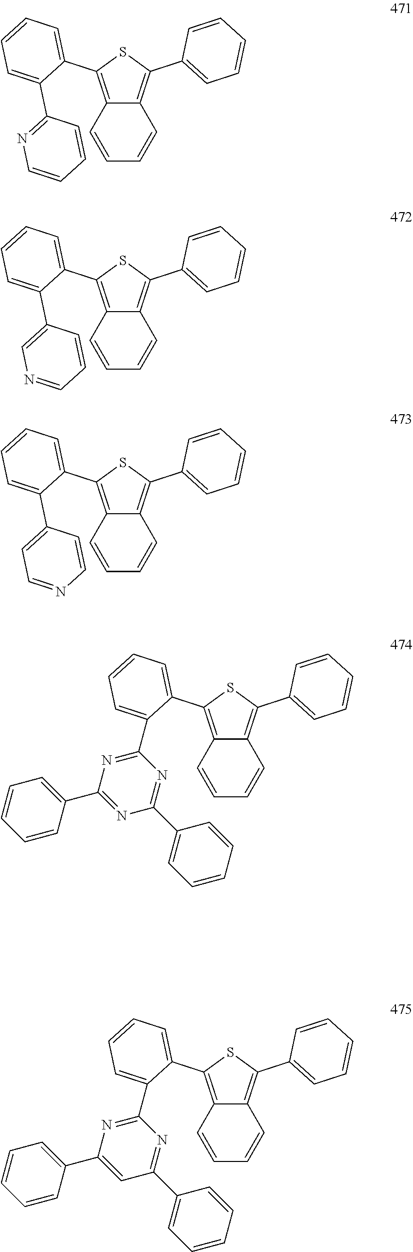 [SMILES: c1ccc(-c2sc(-c3ccccc3-c3ccccn3)c3ccccc23)cc1 ; c1ccc(-c2sc(-c3ccccc3-c3cccnc3)c3ccccc23)cc1 ; c1ccc(-c2sc(-c3ccccc3-c3ccncc3)c3ccccc23)cc1 ; c1ccc(-c2nc(-c3ccccc3)nc(-c3ccccc3-c3sc(-c4ccccc4)c4ccccc34)n2)cc1 ; c1ccc(-c2cc(-c3ccccc3)nc(-c3ccccc3-c3sc(-c4ccccc4)c4ccccc34)n2)cc1]

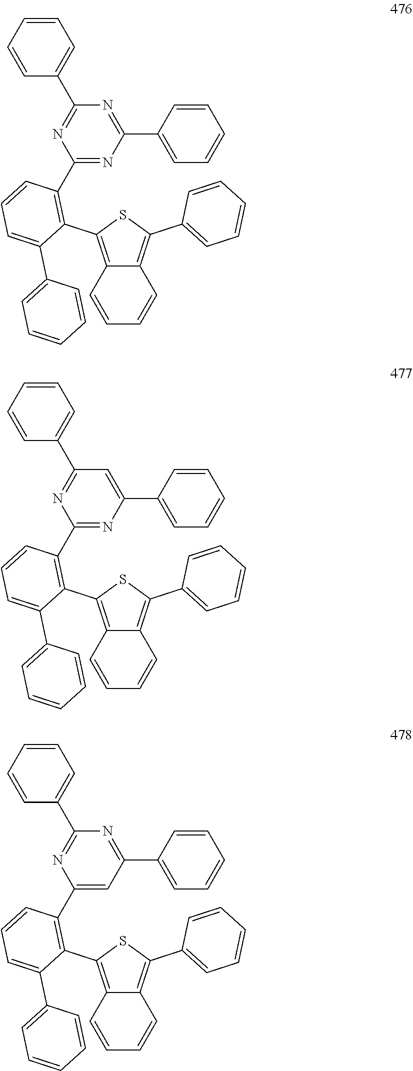

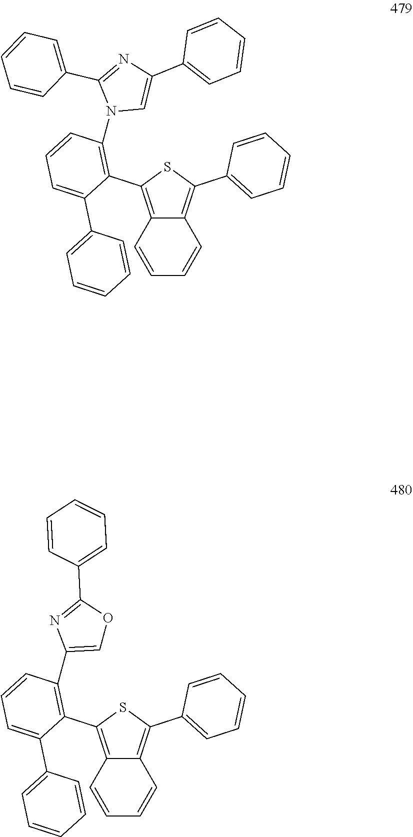 [SMILES: c1ccc(-c2cn(-c3cccc(-c4ccccc4)c3-c3sc(-c4ccccc4)c4ccccc34)c(-c3ccccc3)n2)cc1 ; c1ccc(-c2nc(-c3cccc(-c4ccccc4)c3-c3sc(-c4ccccc4)c4ccccc34)co2)cc1]

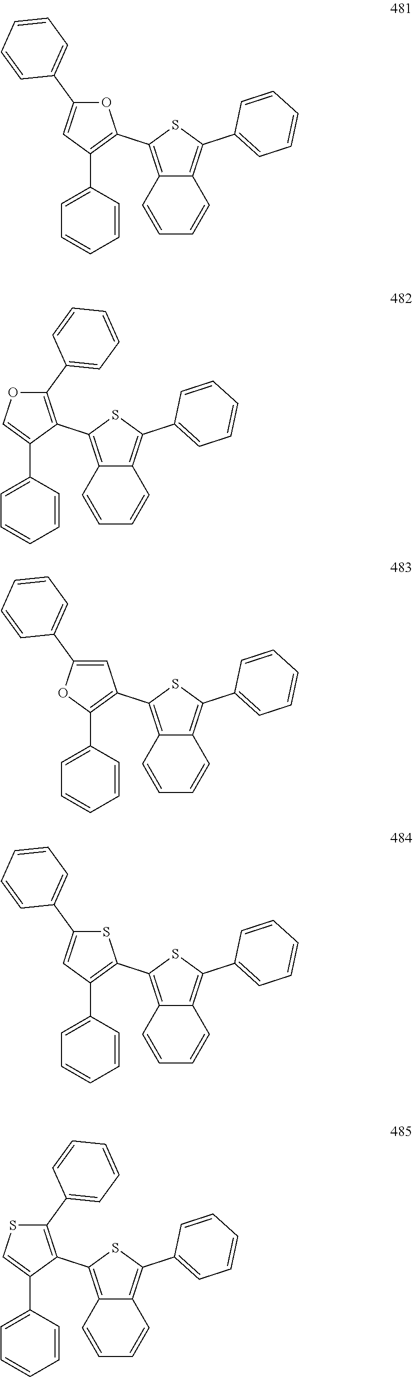

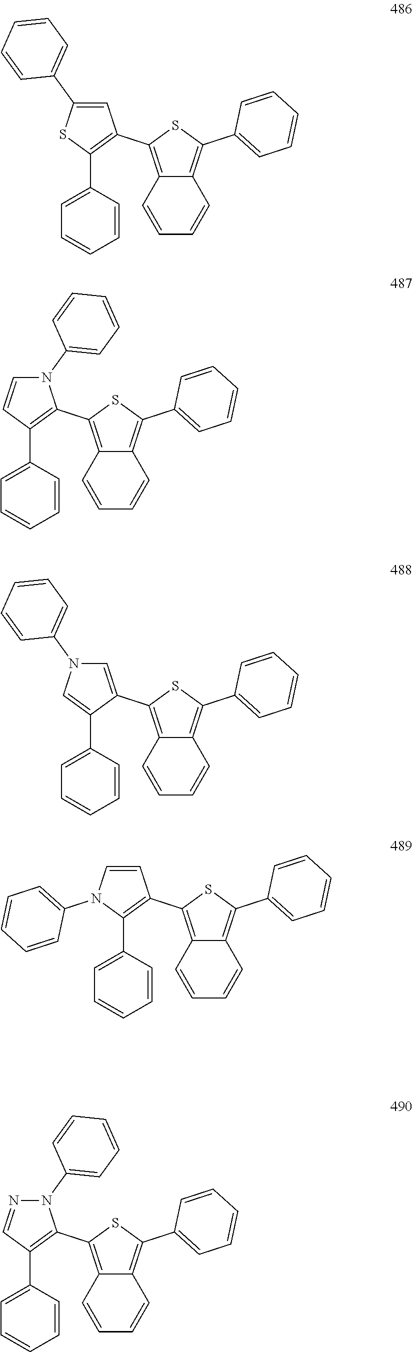

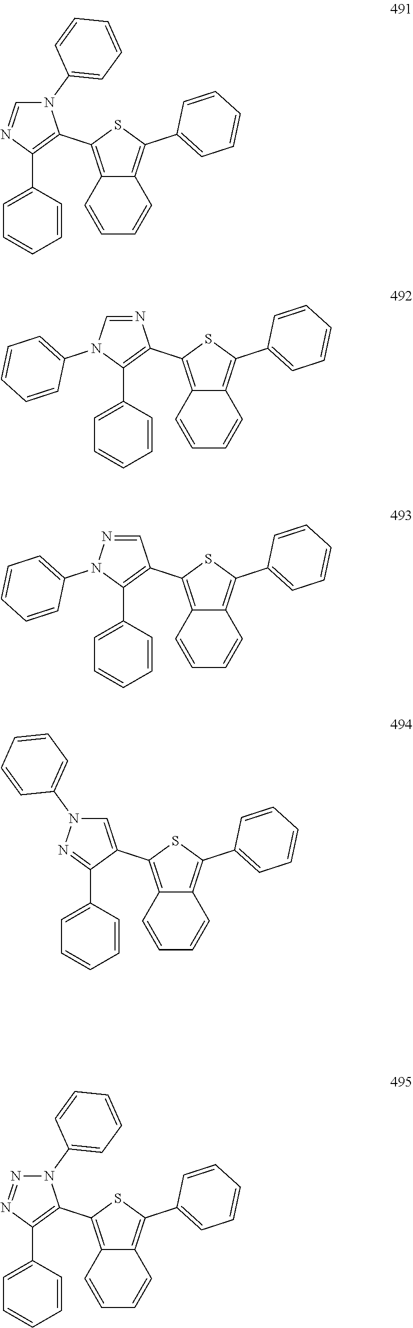 [SMILES: c1ccc(-c2ncn(-c3ccccc3)c2-c2sc(-c3ccccc3)c3ccccc23)cc1 ; c1ccc(-c2sc(-c3ncn(-c4ccccc4)c3-c3ccccc3)c3ccccc23)cc1 ; c1ccc(-c2sc(-c3cnn(-c4ccccc4)c3-c3ccccc3)c3ccccc23)cc1 ; c1ccc(-c2nn(-c3ccccc3)cc2-c2sc(-c3ccccc3)c3ccccc23)cc1 ; c1ccc(-c2nnn(-c3ccccc3)c2-c2sc(-c3ccccc3)c3ccccc23)cc1]

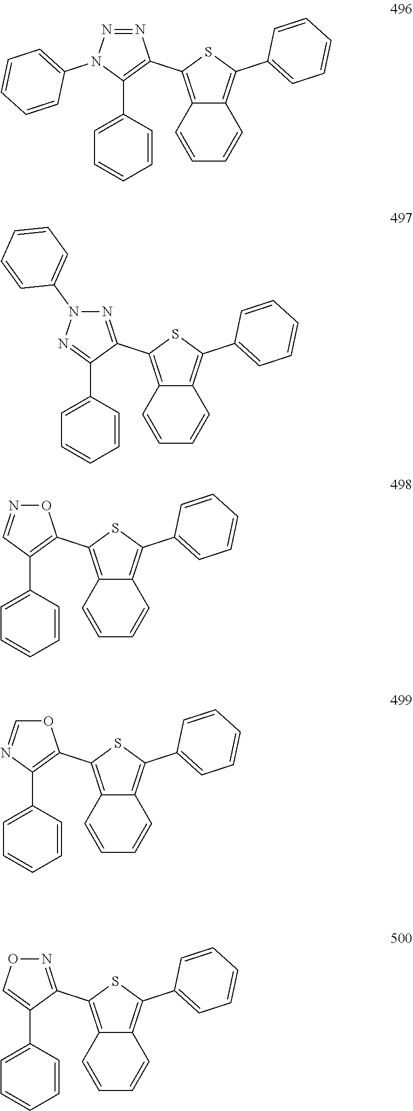 [SMILES: c1ccc(-c2sc(-c3nnn(-c4ccccc4)c3-c3ccccc3)c3ccccc23)cc1 ; c1ccc(-c2nn(-c3ccccc3)nc2-c2sc(-c3ccccc3)c3ccccc23)cc1 ; c1ccc(-c2cnoc2-c2sc(-c3ccccc3)c3ccccc23)cc1 ; c1ccc(-c2ncoc2-c2sc(-c3ccccc3)c3ccccc23)cc1 ; c1ccc(-c2conc2-c2sc(-c3ccccc3)c3ccccc23)cc1]

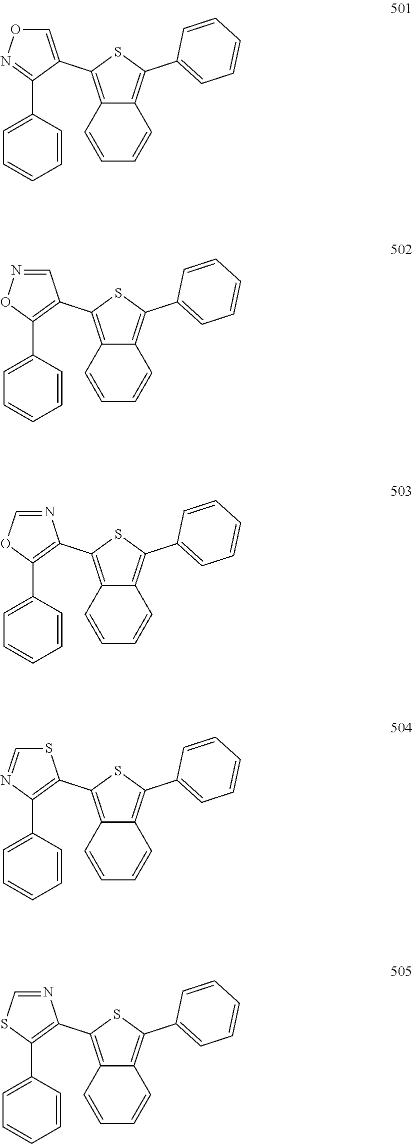 [SMILES: c1ccc(-c2nocc2-c2sc(-c3ccccc3)c3ccccc23)cc1 ; c1ccc(-c2oncc2-c2sc(-c3ccccc3)c3ccccc23)cc1 ; c1ccc(-c2ocnc2-c2sc(-c3ccccc3)c3ccccc23)cc1 ; c1ccc(-c2ncsc2-c2sc(-c3ccccc3)c3ccccc23)cc1 ; c1ccc(-c2scnc2-c2sc(-c3ccccc3)c3ccccc23)cc1]

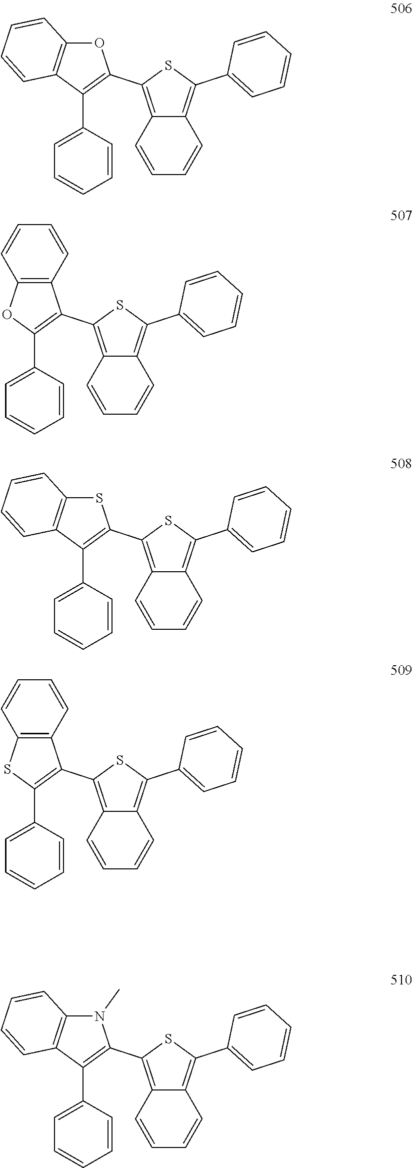

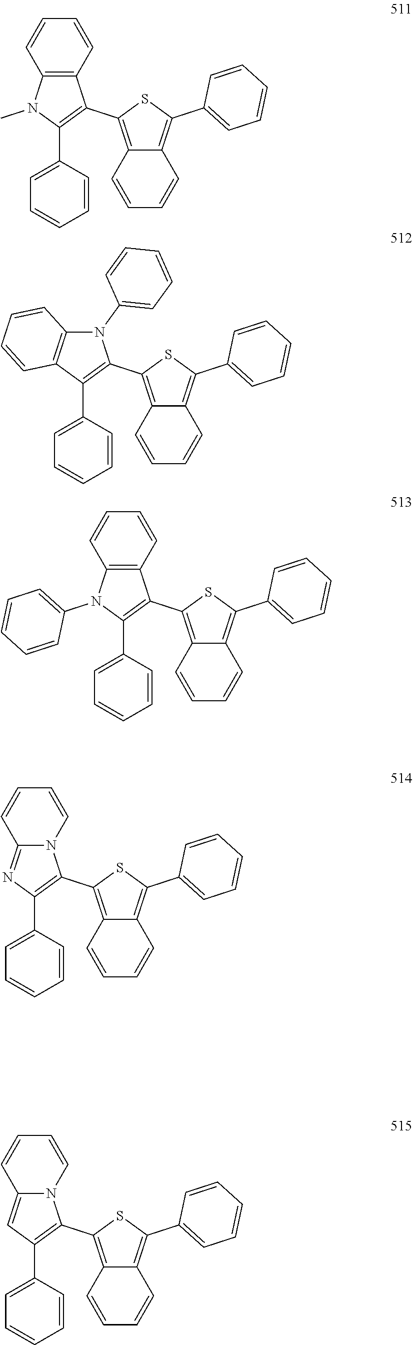

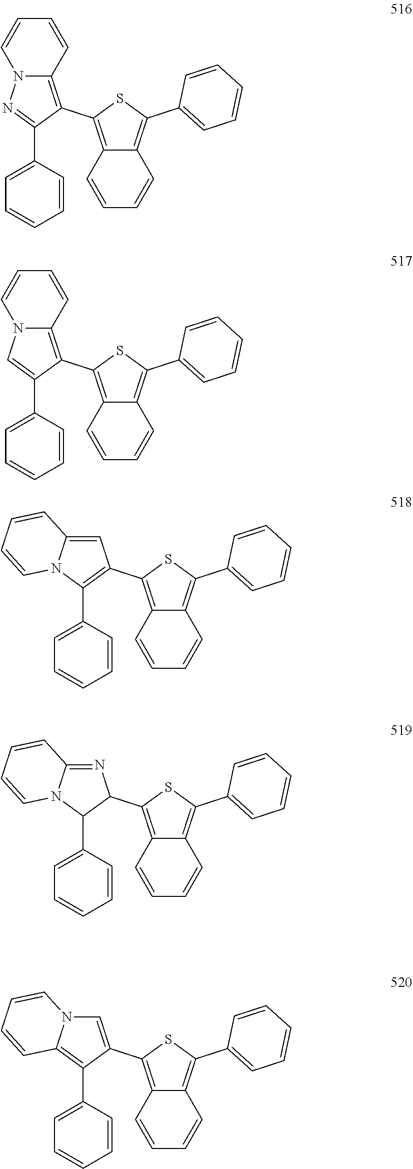

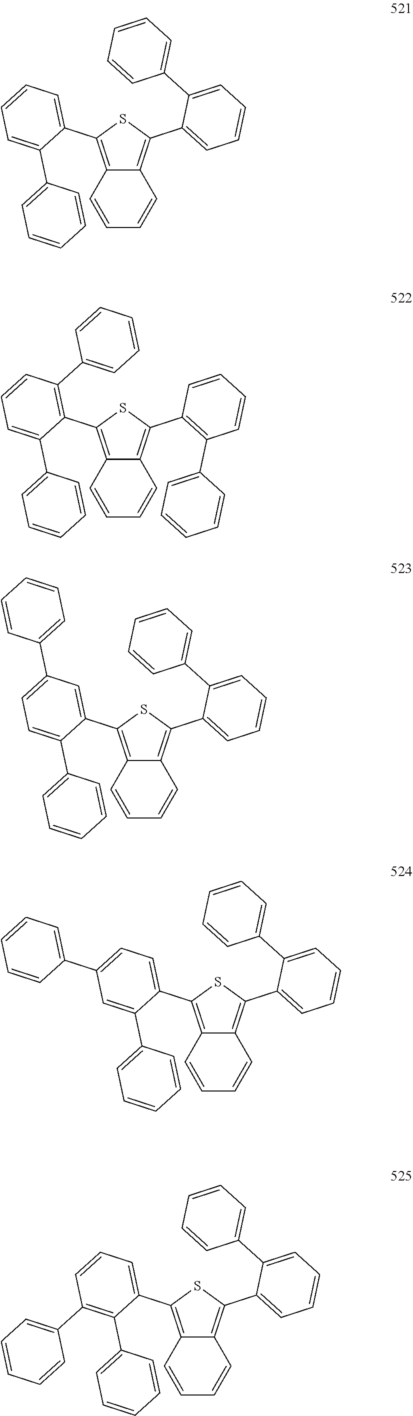 [SMILES: c1ccc(-c2ccccc2-c2sc(-c3ccccc3-c3ccccc3)c3ccccc23)cc1 ; c1ccc(-c2ccccc2-c2sc(-c3c(-c4ccccc4)cccc3-c3ccccc3)c3ccccc23)cc1 ; c1ccc(-c2ccc(-c3ccccc3)c(-c3sc(-c4ccccc4-c4ccccc4)c4ccccc34)c2)cc1 ; c1ccc(-c2ccc(-c3sc(-c4ccccc4-c4ccccc4)c4ccccc34)c(-c3ccccc3)c2)cc1 ; c1ccc(-c2ccccc2-c2sc(-c3cccc(-c4ccccc4)c3-c3ccccc3)c3ccccc23)cc1]

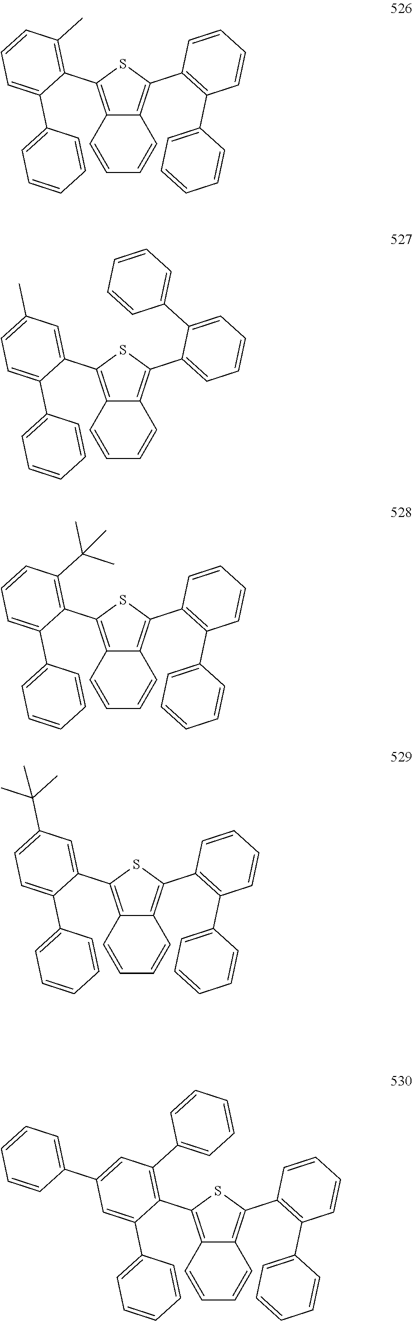

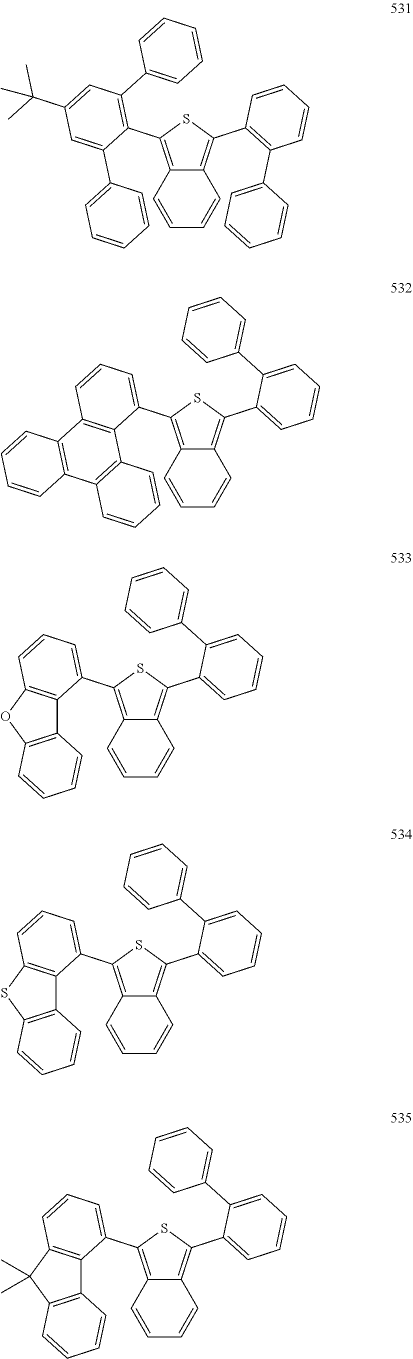 [SMILES: CC(C)(C)c1cc(-c2ccccc2)c(-c2sc(-c3ccccc3-c3ccccc3)c3ccccc23)c(-c2ccccc2)c1 ; c1ccc(-c2ccccc2-c2sc(-c3cccc4c5ccccc5c5ccccc5c34)c3ccccc23)cc1 ; c1ccc(-c2ccccc2-c2sc(-c3cccc4oc5ccccc5c34)c3ccccc23)cc1 ; c1ccc(-c2ccccc2-c2sc(-c3cccc4sc5ccccc5c34)c3ccccc23)cc1 ; CC1(C)c2ccccc2-c2c(-c3sc(-c4ccccc4-c4ccccc4)c4ccccc34)cccc21]

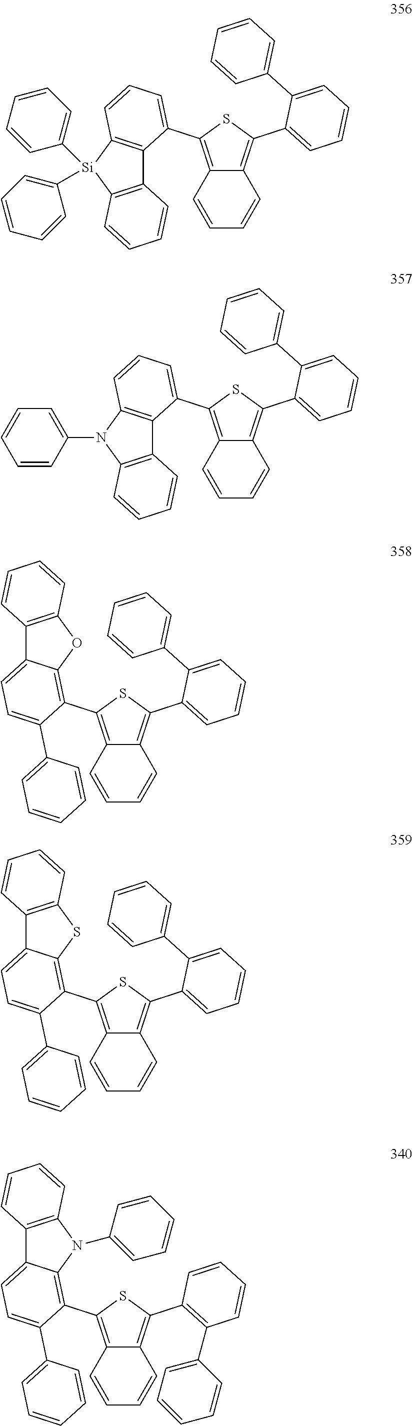

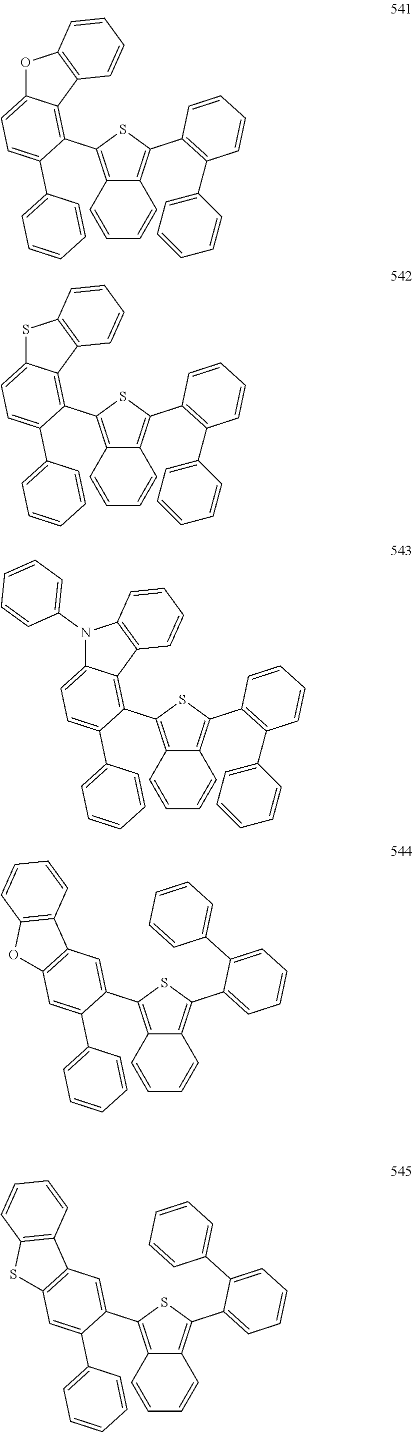

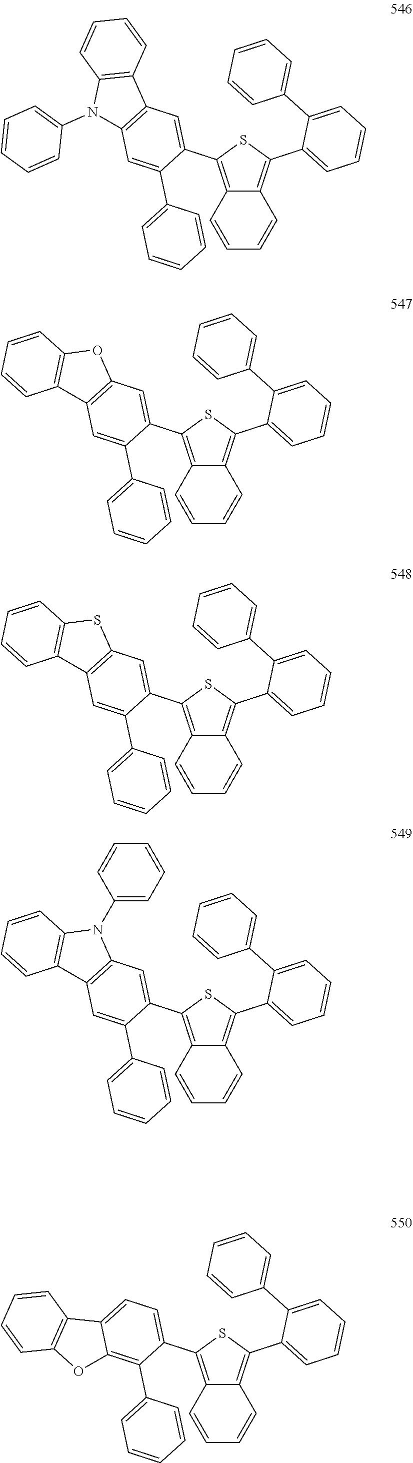

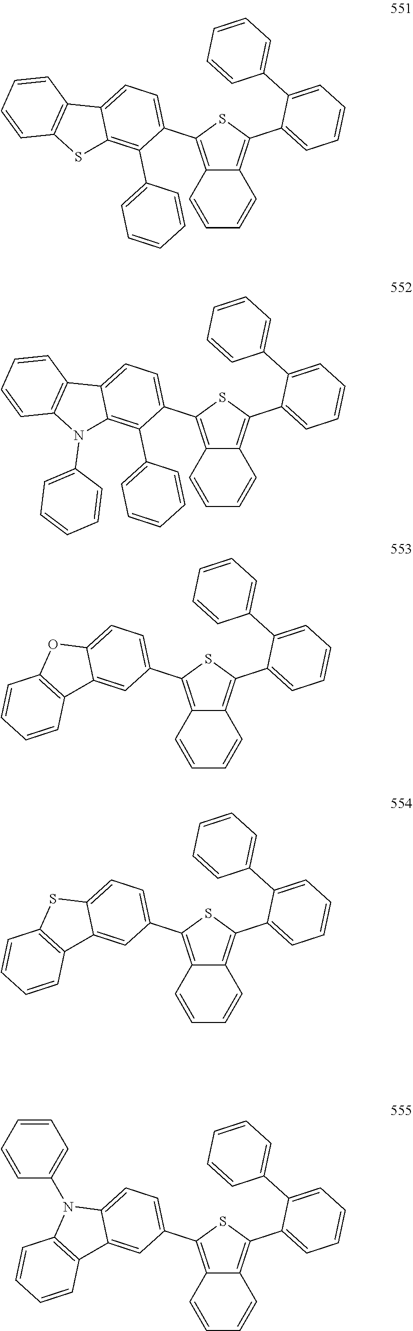

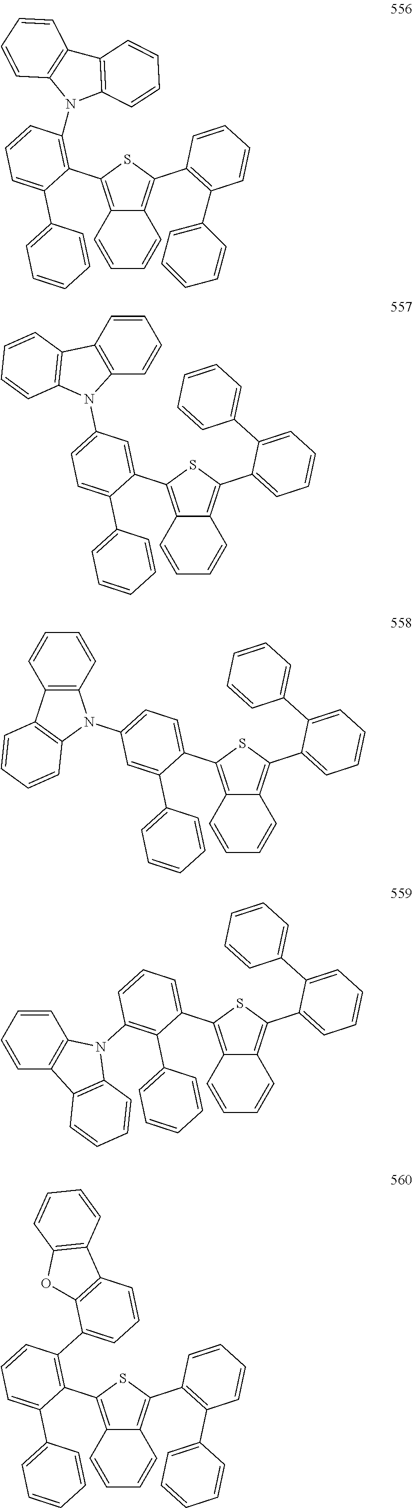

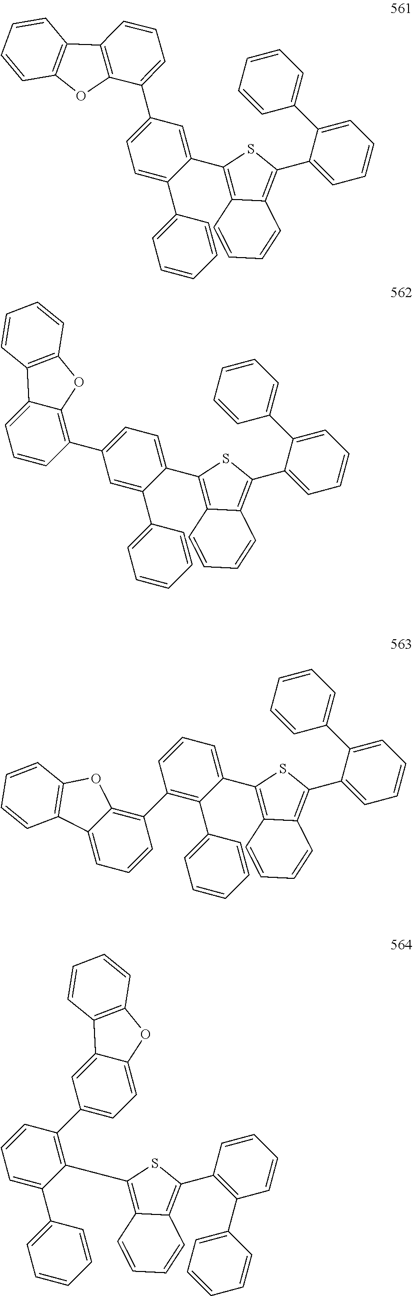 [SMILES: c1ccc(-c2ccccc2-c2sc(-c3cc(-c4cccc5c4oc4ccccc45)ccc3-c3ccccc3)c3ccccc23)cc1 ; c1ccc(-c2ccccc2-c2sc(-c3ccc(-c4cccc5c4oc4ccccc45)cc3-c3ccccc3)c3ccccc23)cc1 ; c1ccc(-c2ccccc2-c2sc(-c3cccc(-c4cccc5c4oc4ccccc45)c3-c3ccccc3)c3ccccc23)cc1 ; c1ccc(-c2ccccc2-c2sc(-c3c(-c4ccccc4)cccc3-c3ccc4oc5ccccc5c4c3)c3ccccc23)cc1]

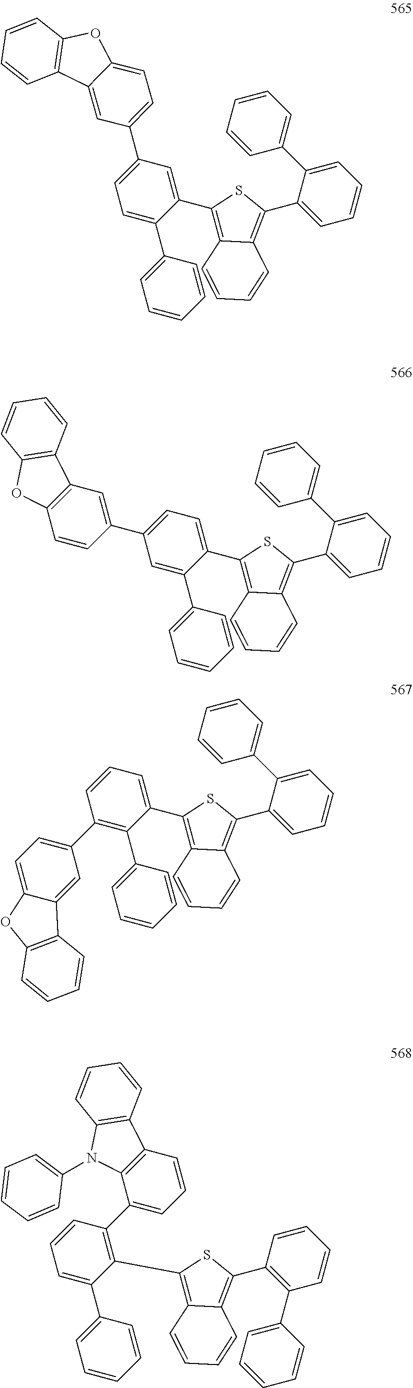

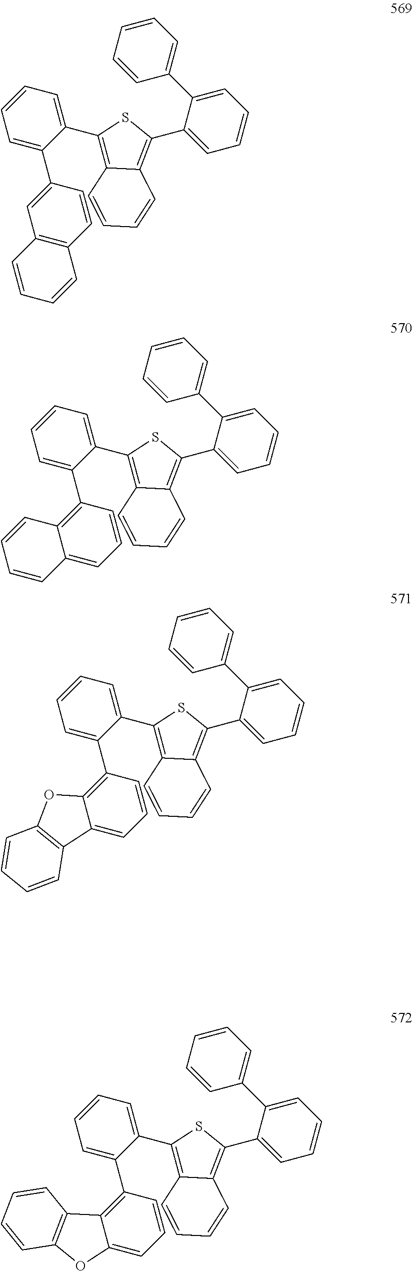 [SMILES: c1ccc(-c2ccccc2-c2sc(-c3ccccc3-c3ccc4ccccc4c3)c3ccccc23)cc1 ; c1ccc(-c2ccccc2-c2sc(-c3ccccc3-c3cccc4ccccc34)c3ccccc23)cc1 ; c1ccc(-c2ccccc2-c2sc(-c3ccccc3-c3cccc4c3oc3ccccc34)c3ccccc23)cc1 ; c1ccc(-c2ccccc2-c2sc(-c3ccccc3-c3cccc4oc5ccccc5c34)c3ccccc23)cc1]

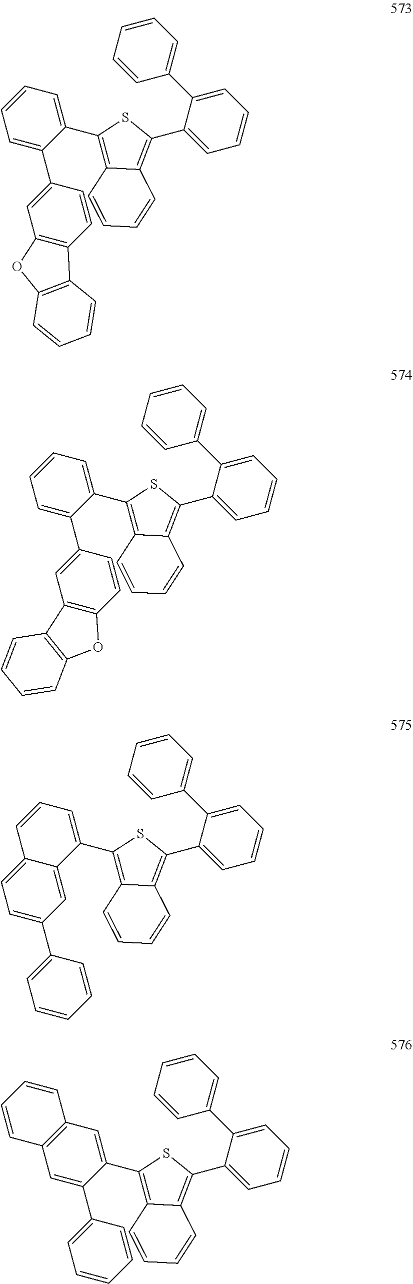 [SMILES: c1ccc(-c2ccccc2-c2sc(-c3ccccc3-c3ccc4c(c3)oc3ccccc34)c3ccccc23)cc1 ; c1ccc(-c2ccccc2-c2sc(-c3ccccc3-c3ccc4oc5ccccc5c4c3)c3ccccc23)cc1 ; c1ccc(-c2ccc3cccc(-c4sc(-c5ccccc5-c5ccccc5)c5ccccc45)c3c2)cc1 ; c1ccc(-c2ccccc2-c2sc(-c3cc4ccccc4cc3-c3ccccc3)c3ccccc23)cc1]

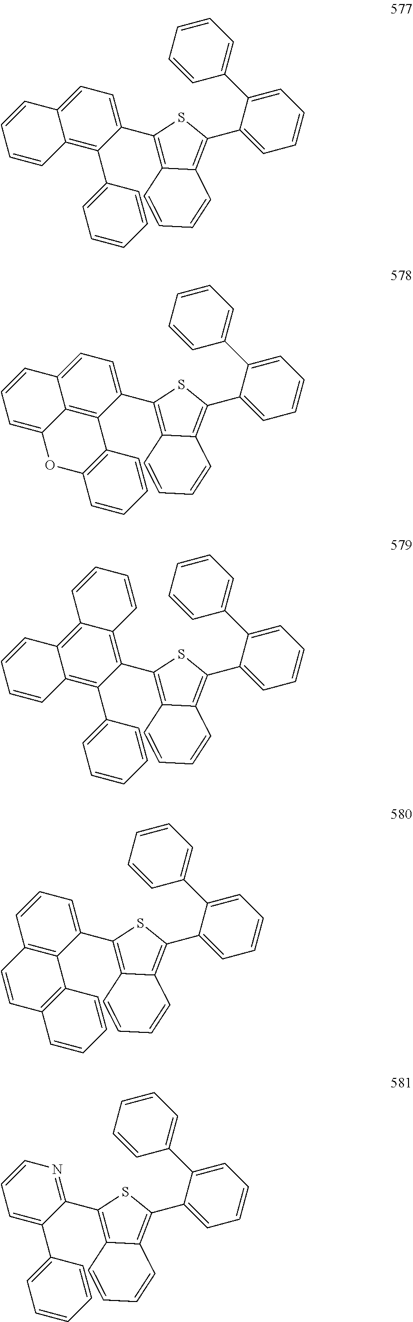 [SMILES: c1ccc(-c2ccccc2-c2sc(-c3ccc4ccccc4c3-c3ccccc3)c3ccccc23)cc1 ; c1ccc(-c2ccccc2-c2sc(-c3ccc4cccc5c4c3-c3ccccc3O5)c3ccccc23)cc1 ; c1ccc(-c2ccccc2-c2sc(-c3c(-c4ccccc4)c4ccccc4c4ccccc34)c3ccccc23)cc1 ; c1ccc(-c2ccccc2-c2sc(-c3cccc4ccc5ccccc5c34)c3ccccc23)cc1 ; c1ccc(-c2ccccc2-c2sc(-c3ncccc3-c3ccccc3)c3ccccc23)cc1]

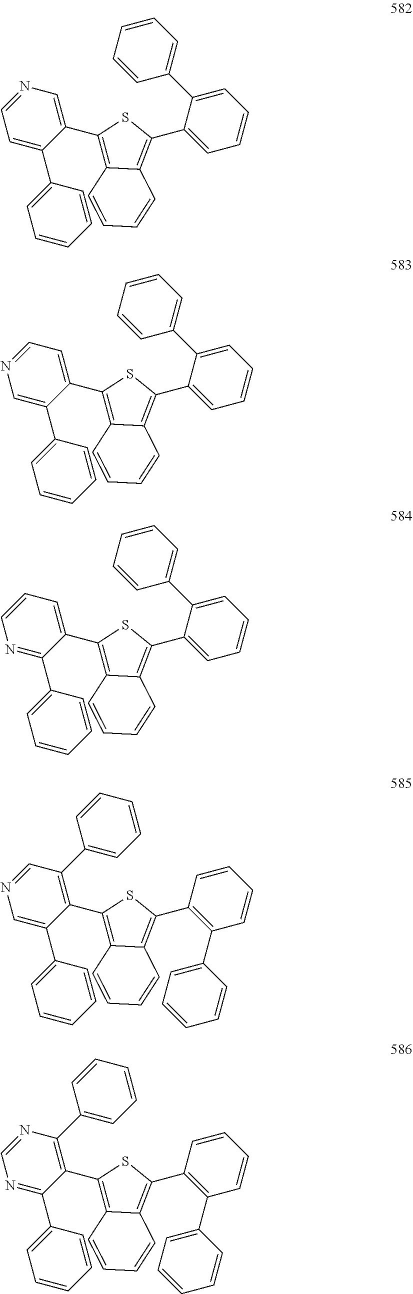

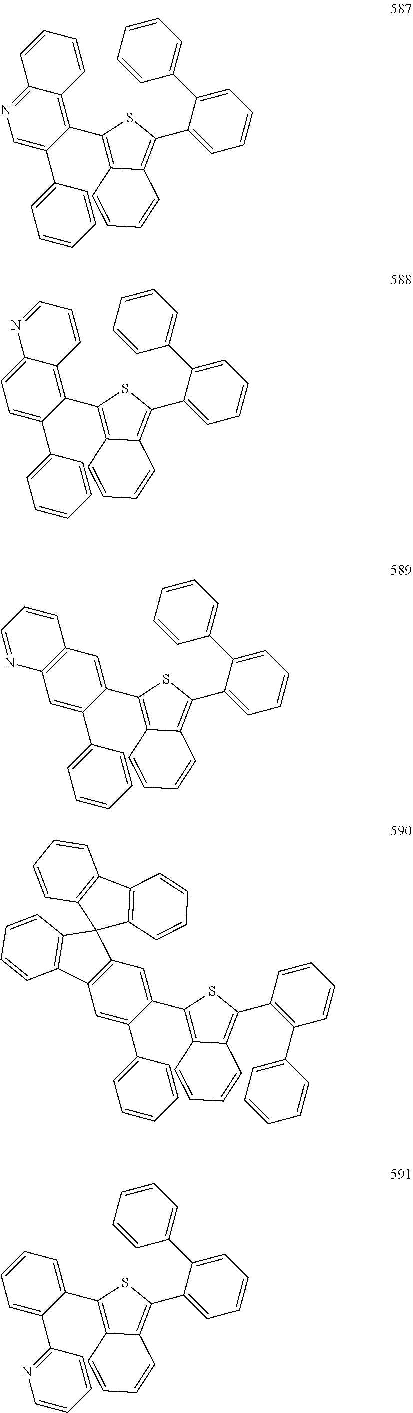

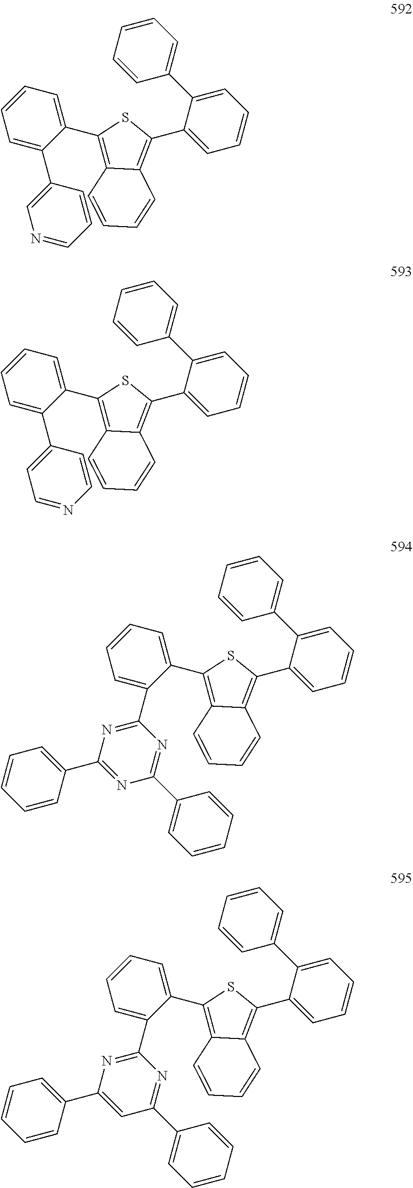 [SMILES: c1ccc(-c2ccccc2-c2sc(-c3ccccc3-c3cccnc3)c3ccccc23)cc1 ; c1ccc(-c2ccccc2-c2sc(-c3ccccc3-c3ccncc3)c3ccccc23)cc1 ; c1ccc(-c2nc(-c3ccccc3)nc(-c3ccccc3-c3sc(-c4ccccc4-c4ccccc4)c4ccccc34)n2)cc1 ; c1ccc(-c2cc(-c3ccccc3)nc(-c3ccccc3-c3sc(-c4ccccc4-c4ccccc4)c4ccccc34)n2)cc1]

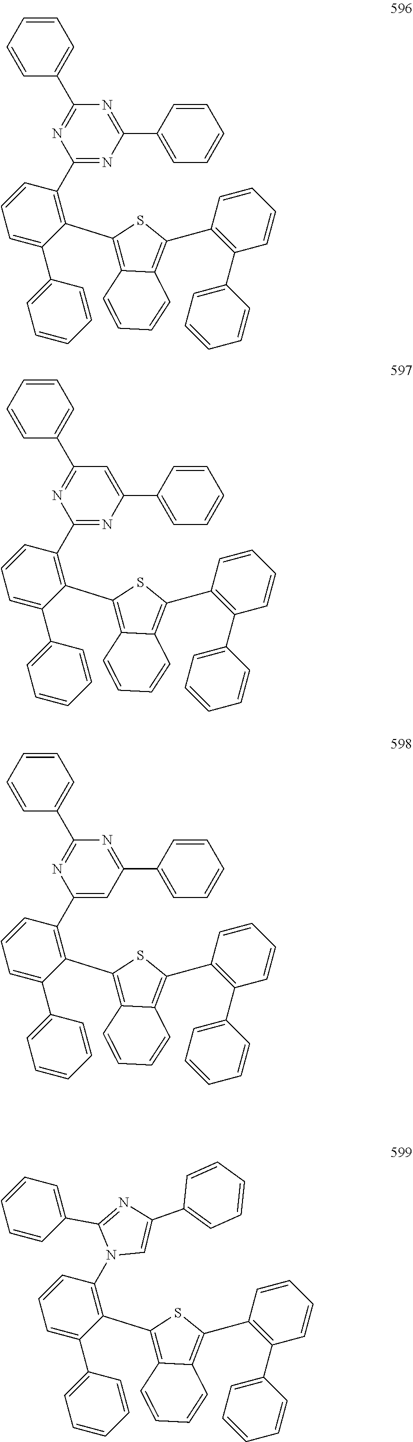

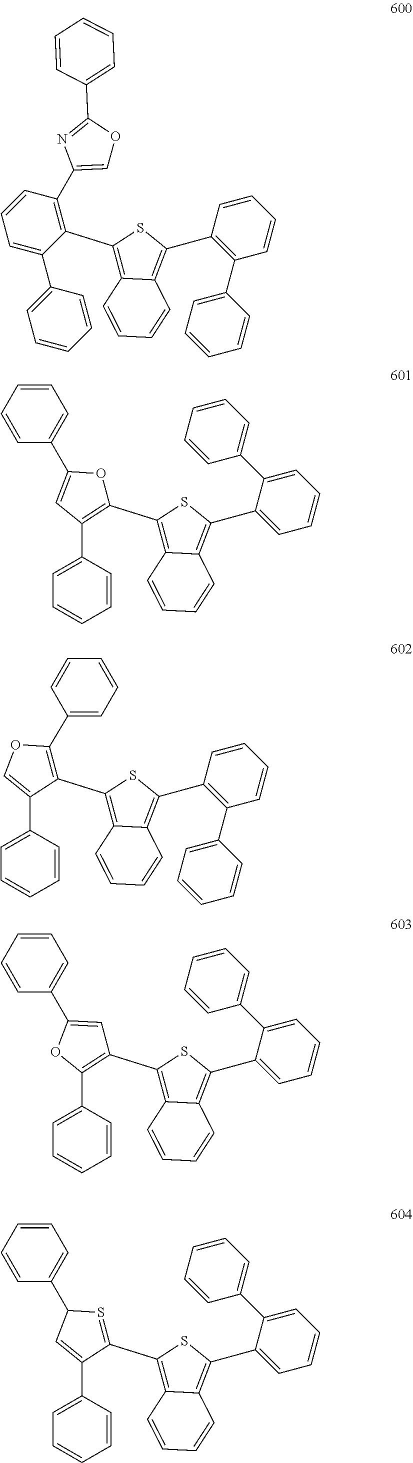

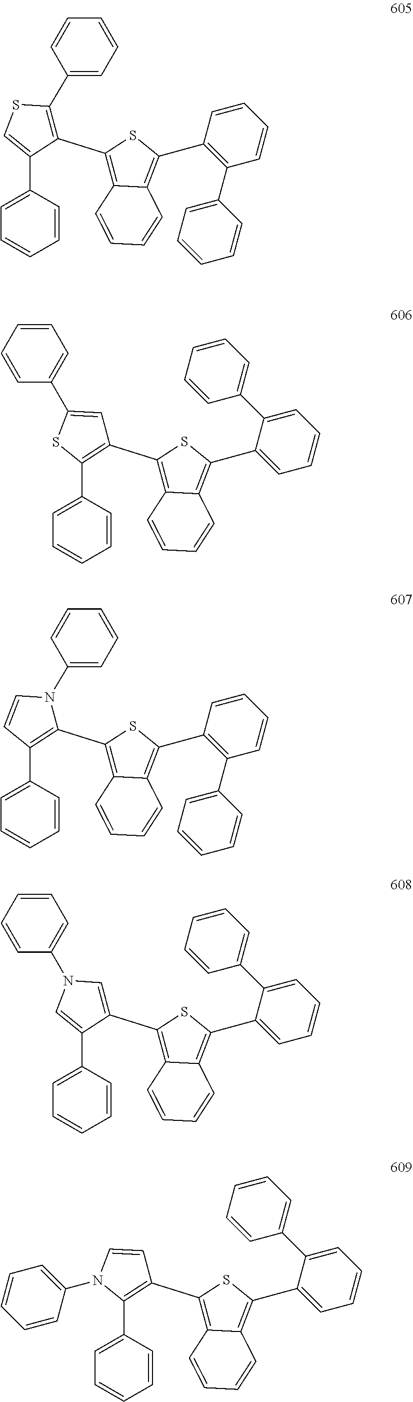 [SMILES: c1ccc(-c2ccccc2-c2sc(-c3c(-c4ccccc4)csc3-c3ccccc3)c3ccccc23)cc1 ; c1ccc(-c2cc(-c3sc(-c4ccccc4-c4ccccc4)c4ccccc34)c(-c3ccccc3)s2)cc1 ; c1ccc(-c2ccccc2-c2sc(-c3c(-c4ccccc4)ccn3-c3ccccc3)c3ccccc23)cc1 ; c1ccc(-c2ccccc2-c2sc(-c3cn(-c4ccccc4)cc3-c3ccccc3)c3ccccc23)cc1 ; c1ccc(-c2ccccc2-c2sc(-c3ccn(-c4ccccc4)c3-c3ccccc3)c3ccccc23)cc1]

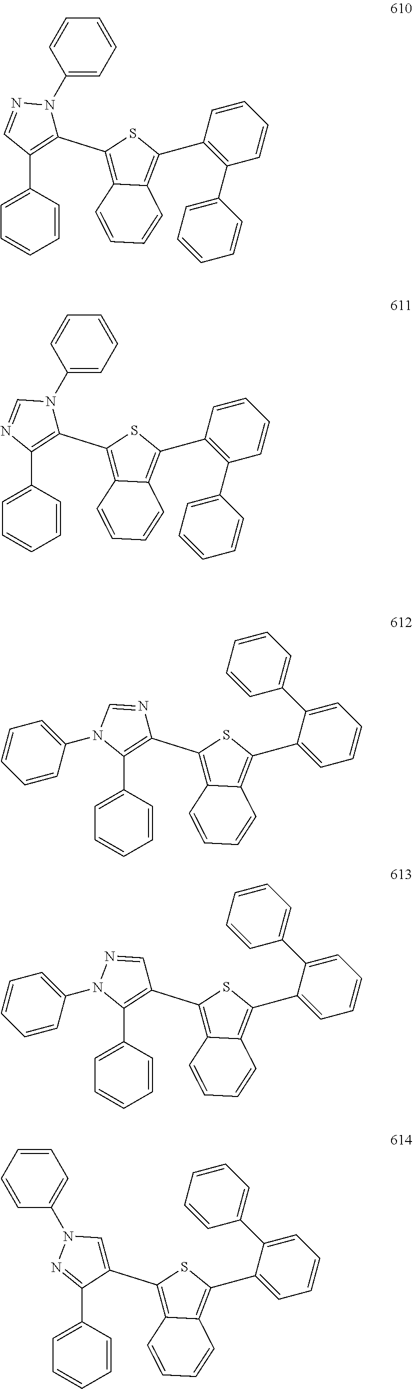 [SMILES: c1ccc(-c2ccccc2-c2sc(-c3c(-c4ccccc4)cnn3-c3ccccc3)c3ccccc23)cc1 ; c1ccc(-c2ccccc2-c2sc(-c3c(-c4ccccc4)ncn3-c3ccccc3)c3ccccc23)cc1 ; c1ccc(-c2ccccc2-c2sc(-c3ncn(-c4ccccc4)c3-c3ccccc3)c3ccccc23)cc1 ; c1ccc(-c2ccccc2-c2sc(-c3cnn(-c4ccccc4)c3-c3ccccc3)c3ccccc23)cc1 ; c1ccc(-c2ccccc2-c2sc(-c3cn(-c4ccccc4)nc3-c3ccccc3)c3ccccc23)cc1]

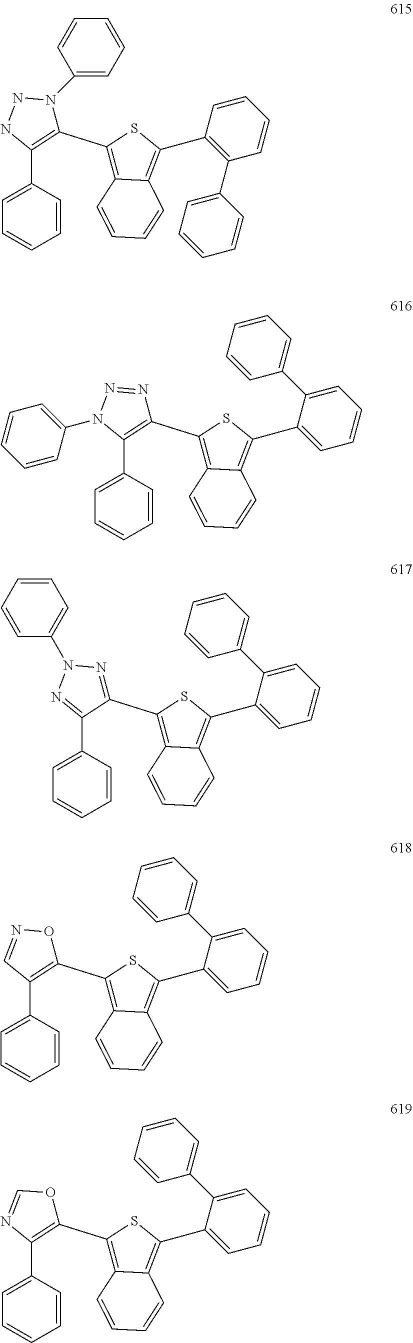 [SMILES: c1ccc(C2=C(c3sc(-c4ccccc4-c4ccccc4)c4ccccc34)N(c3ccccc3)NN2)cc1 ; c1ccc(-c2ccccc2-c2sc(-c3nnn(-c4ccccc4)c3-c3ccccc3)c3ccccc23)cc1 ; c1ccc(-c2ccccc2-c2sc(-c3nn(-c4ccccc4)nc3-c3ccccc3)c3ccccc23)cc1 ; c1ccc(-c2ccccc2-c2sc(-c3oncc3-c3ccccc3)c3ccccc23)cc1 ; c1ccc(-c2ccccc2-c2sc(-c3ocnc3-c3ccccc3)c3ccccc23)cc1]

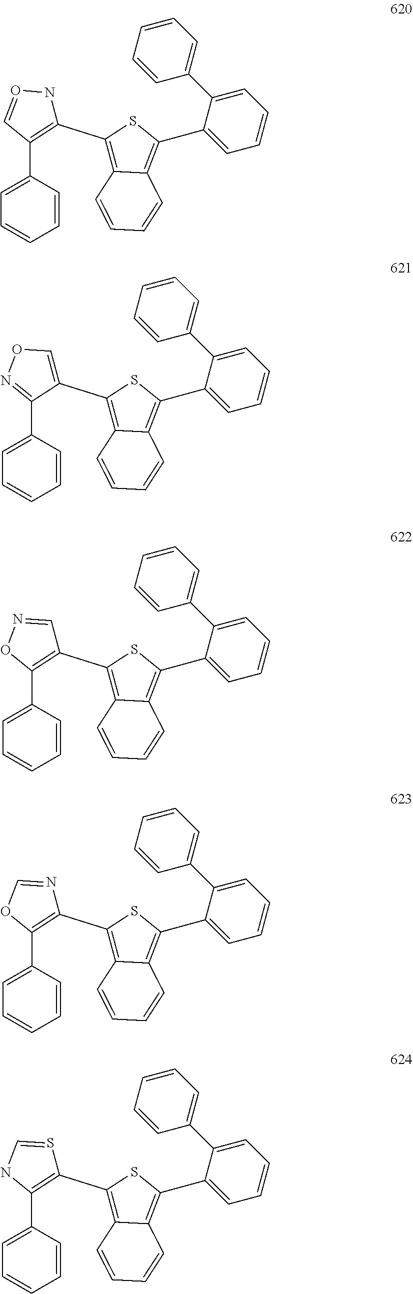

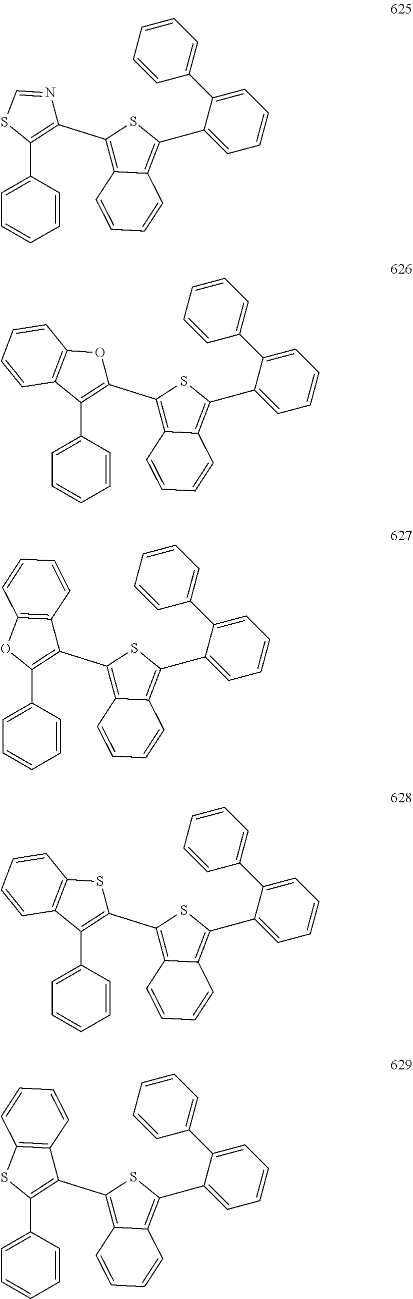 [SMILES: c1ccc(-c2ccccc2-c2sc(-c3ncsc3-c3ccccc3)c3ccccc23)cc1 ; c1ccc(-c2ccccc2-c2sc(-c3oc4ccccc4c3-c3ccccc3)c3ccccc23)cc1 ; c1ccc(-c2ccccc2-c2sc(-c3c(-c4ccccc4)oc4ccccc34)c3ccccc23)cc1 ; c1ccc(-c2ccccc2-c2sc(-c3sc4ccccc4c3-c3ccccc3)c3ccccc23)cc1 ; c1ccc(-c2ccccc2-c2sc(-c3c(-c4ccccc4)sc4ccccc34)c3ccccc23)cc1]

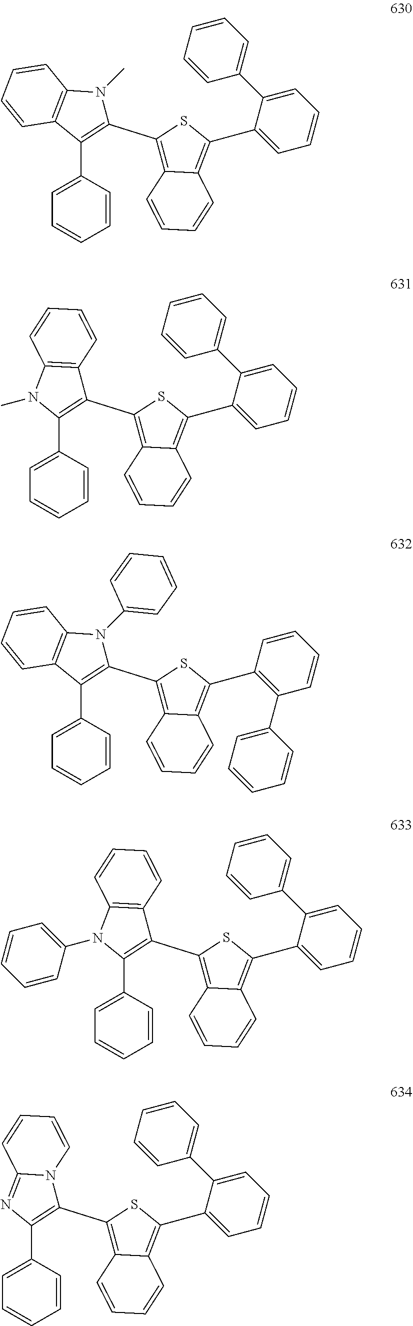

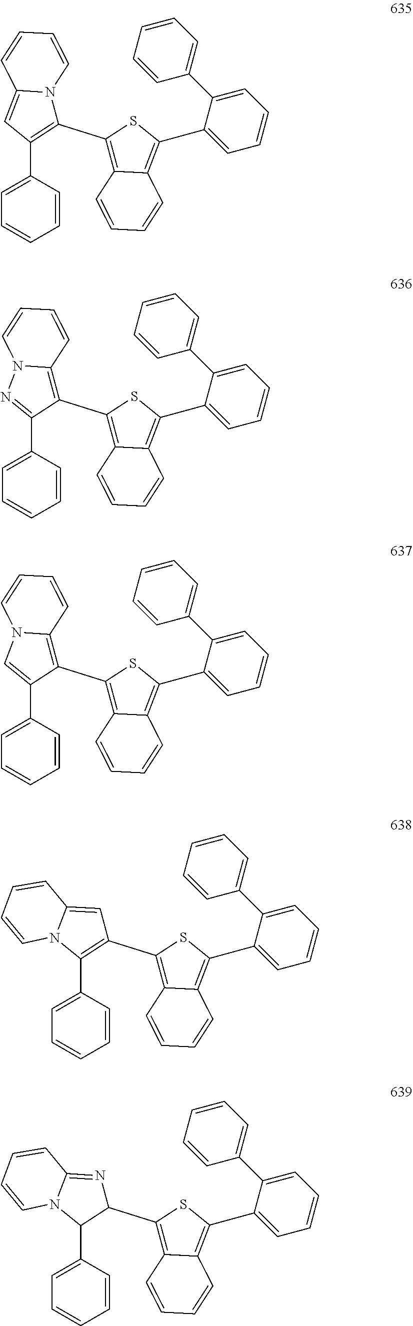 [SMILES: c1ccc(-c2ccccc2-c2sc(-c3c(-c4ccccc4)cc4ccccn34)c3ccccc23)cc1 ; c1ccc(-c2ccccc2-c2sc(-c3c(-c4ccccc4)nn4ccccc34)c3ccccc23)cc1 ; c1ccc(-c2ccccc2-c2sc(-c3c(-c4ccccc4)cn4ccccc34)c3ccccc23)cc1 ; c1ccc(-c2ccccc2-c2sc(-c3cc4ccccn4c3-c3ccccc3)c3ccccc23)cc1 ; C1=CC2=NC(c3sc(-c4ccccc4-c4ccccc4)c4ccccc34)C(c3ccccc3)N2C=C1]

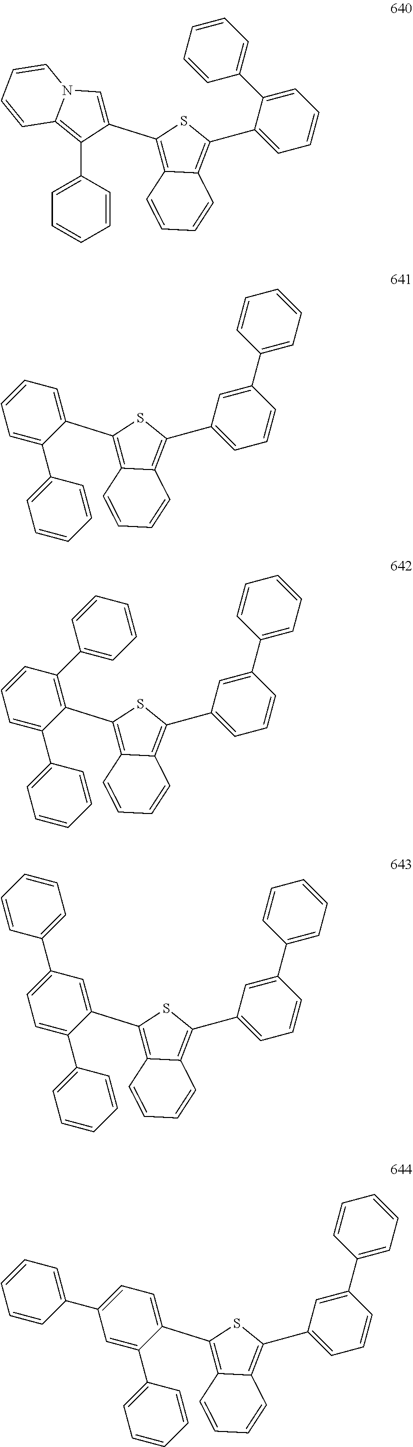 [SMILES: c1ccc(-c2ccccc2-c2sc(-c3cn4ccccc4c3-c3ccccc3)c3ccccc23)cc1 ; c1ccc(-c2cccc(-c3sc(-c4ccccc4-c4ccccc4)c4ccccc34)c2)cc1 ; c1ccc(-c2cccc(-c3sc(-c4c(-c5ccccc5)cccc4-c4ccccc4)c4ccccc34)c2)cc1 ; c1ccc(-c2cccc(-c3sc(-c4cc(-c5ccccc5)ccc4-c4ccccc4)c4ccccc34)c2)cc1 ; c1ccc(-c2cccc(-c3sc(-c4ccc(-c5ccccc5)cc4-c4ccccc4)c4ccccc34)c2)cc1]

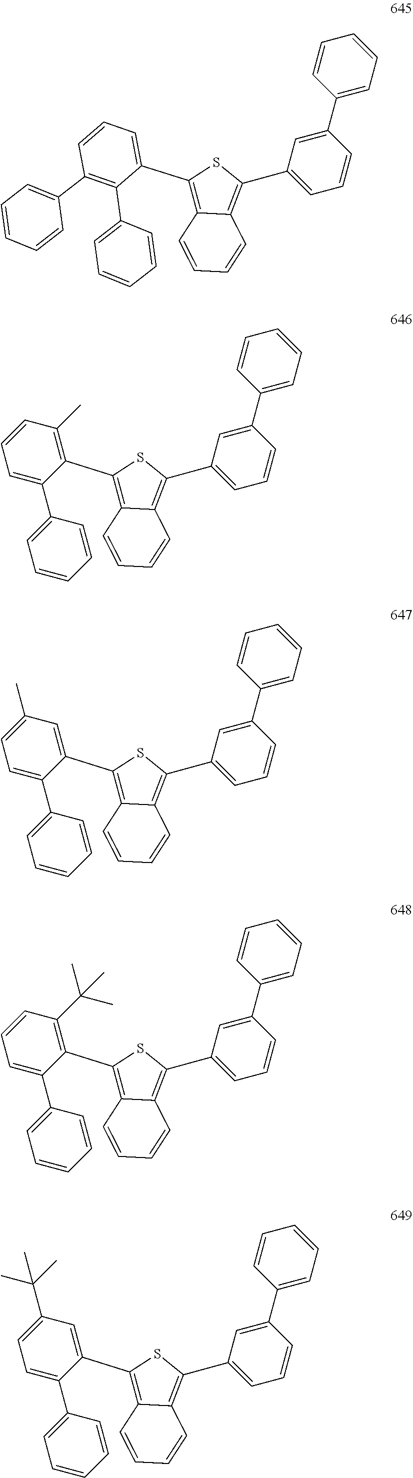 [SMILES: c1ccc(-c2cccc(-c3sc(-c4cccc(-c5ccccc5)c4-c4ccccc4)c4ccccc34)c2)cc1 ; Cc1cccc(-c2ccccc2)c1-c1sc(-c2cccc(-c3ccccc3)c2)c2ccccc12 ; Cc1ccc(-c2ccccc2)c(-c2sc(-c3cccc(-c4ccccc4)c3)c3ccccc23)c1 ; CC(C)(C)c1cccc(-c2ccccc2)c1-c1sc(-c2cccc(-c3ccccc3)c2)c2ccccc12 ; CC(C)(C)c1ccc(-c2ccccc2)c(-c2sc(-c3cccc(-c4ccccc4)c3)c3ccccc23)c1]

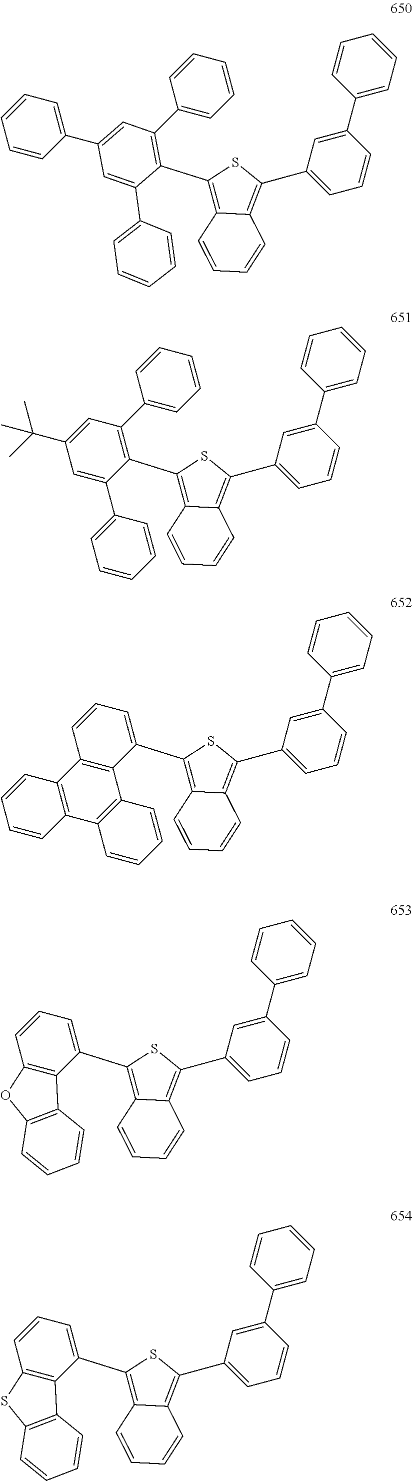 [SMILES: c1ccc(-c2cccc(-c3sc(-c4c(-c5ccccc5)cc(-c5ccccc5)cc4-c4ccccc4)c4ccccc34)c2)cc1 ; CC(C)(C)c1cc(-c2ccccc2)c(-c2sc(-c3cccc(-c4ccccc4)c3)c3ccccc23)c(-c2ccccc2)c1 ; c1ccc(-c2cccc(-c3sc(-c4cccc5c6ccccc6c6ccccc6c45)c4ccccc34)c2)cc1 ; c1ccc(-c2cccc(-c3sc(-c4cccc5oc6ccccc6c45)c4ccccc34)c2)cc1 ; c1ccc(-c2cccc(-c3sc(-c4cccc5sc6ccccc6c45)c4ccccc34)c2)cc1]

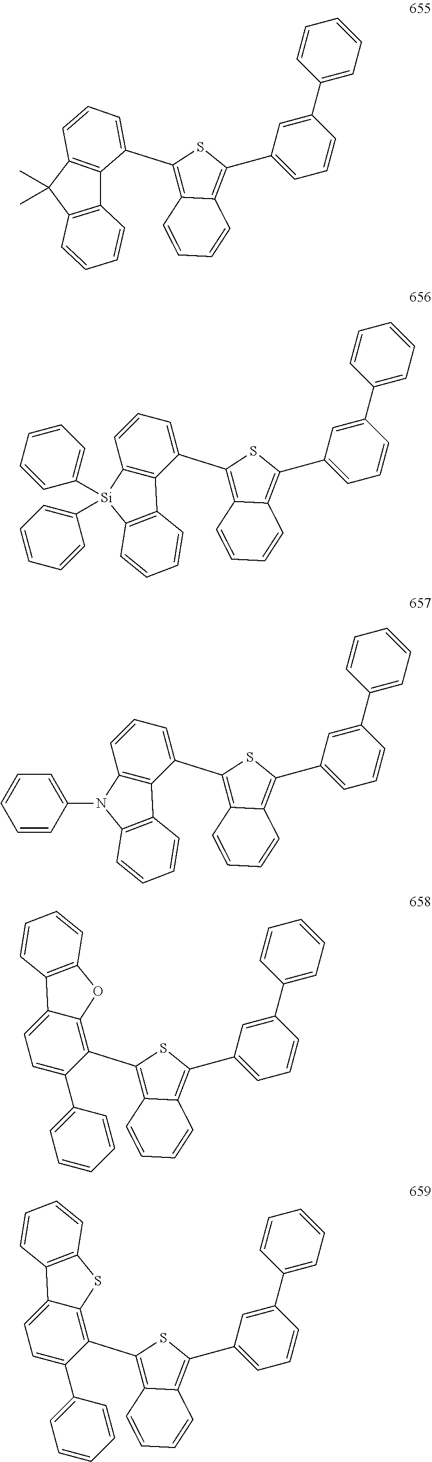

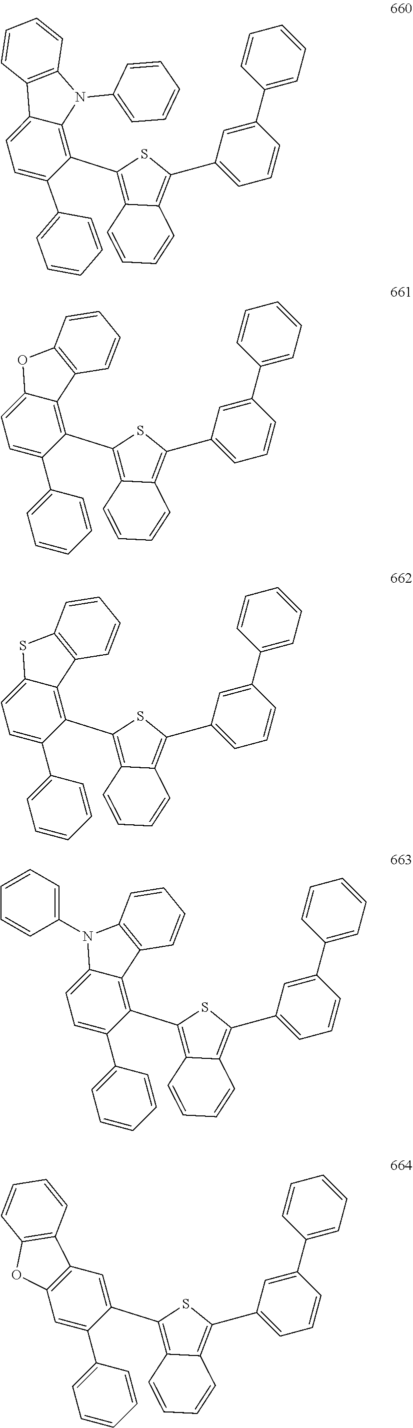

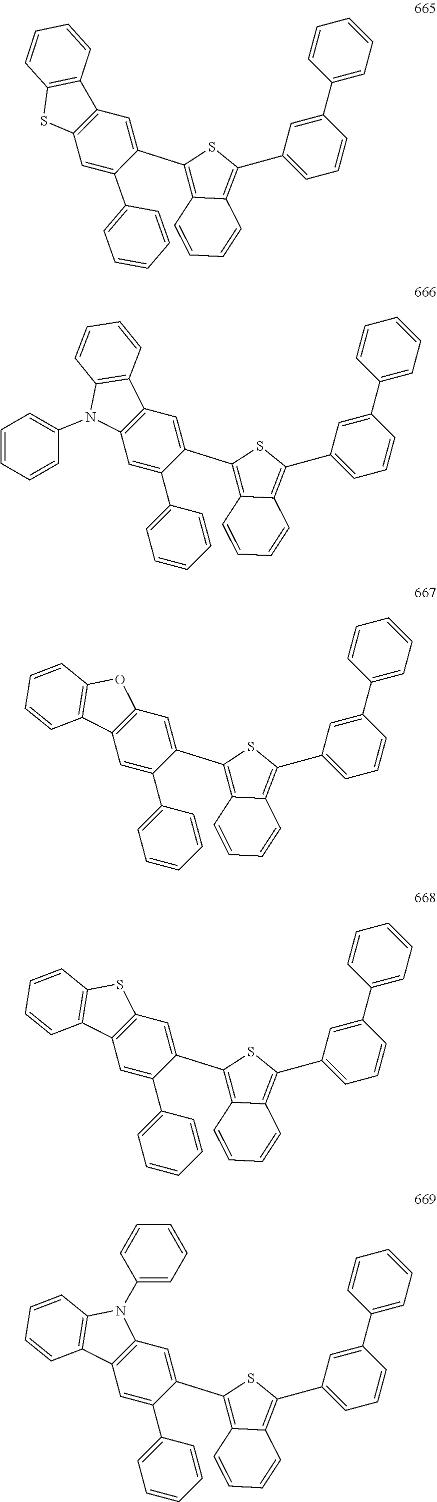 [SMILES: c1ccc(-c2cccc(-c3sc(-c4cc5c(cc4-c4ccccc4)sc4ccccc45)c4ccccc34)c2)cc1 ; c1ccc(-c2cccc(-c3sc(-c4cc5c6ccccc6n(-c6ccccc6)c5cc4-c4ccccc4)c4ccccc34)c2)cc1 ; c1ccc(-c2cccc(-c3sc(-c4cc5oc6ccccc6c5cc4-c4ccccc4)c4ccccc34)c2)cc1 ; c1ccc(-c2cccc(-c3sc(-c4cc5sc6ccccc6c5cc4-c4ccccc4)c4ccccc34)c2)cc1 ; c1ccc(-c2cccc(-c3sc(-c4cc5c(cc4-c4ccccc4)c4ccccc4n5-c4ccccc4)c4ccccc34)c2)cc1]

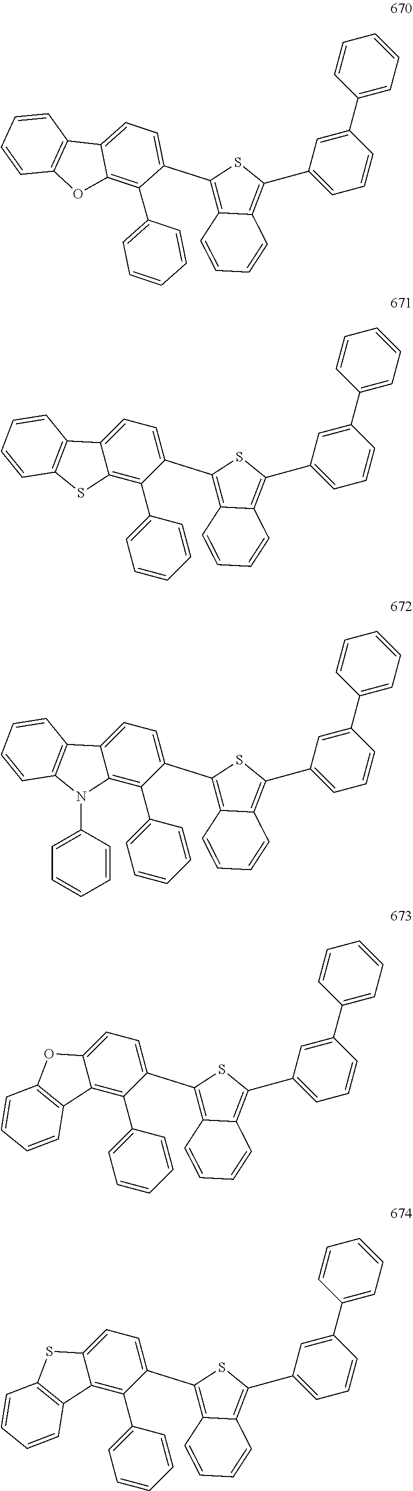 [SMILES: c1ccc(-c2cccc(-c3sc(-c4ccc5c(oc6ccccc65)c4-c4ccccc4)c4ccccc34)c2)cc1 ; c1ccc(-c2cccc(-c3sc(-c4ccc5c(sc6ccccc65)c4-c4ccccc4)c4ccccc34)c2)cc1 ; c1ccc(-c2cccc(-c3sc(-c4ccc5c6ccccc6n(-c6ccccc6)c5c4-c4ccccc4)c4ccccc34)c2)cc1 ; c1ccc(-c2cccc(-c3sc(-c4ccc5oc6ccccc6c5c4-c4ccccc4)c4ccccc34)c2)cc1 ; c1ccc(-c2cccc(-c3sc(-c4ccc5sc6ccccc6c5c4-c4ccccc4)c4ccccc34)c2)cc1]

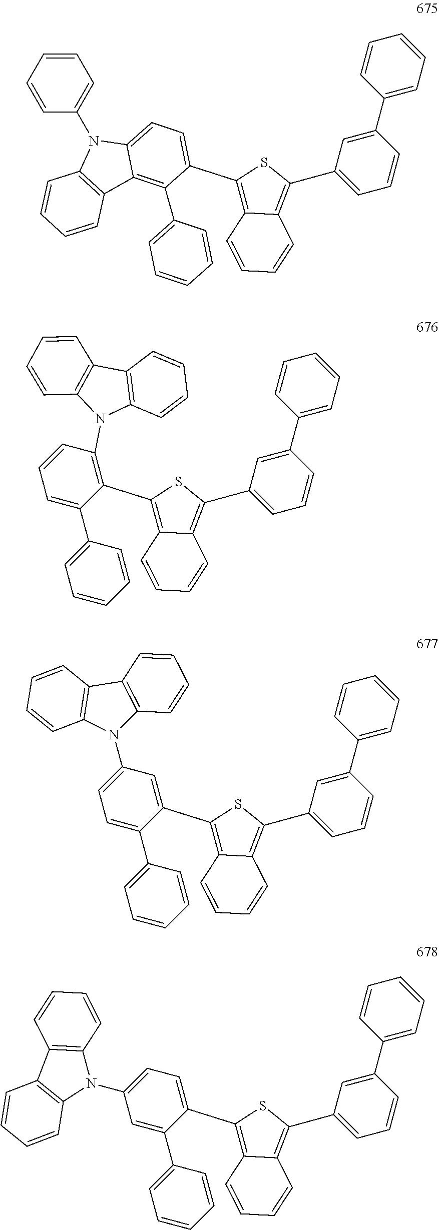 [SMILES: c1ccc(-c2cccc(-c3sc(-c4ccc5c(c4-c4ccccc4)c4ccccc4n5-c4ccccc4)c4ccccc34)c2)cc1 ; c1ccc(-c2cccc(-c3sc(-c4c(-c5ccccc5)cccc4-n4c5ccccc5c5ccccc54)c4ccccc34)c2)cc1 ; c1ccc(-c2cccc(-c3sc(-c4cc(-n5c6ccccc6c6ccccc65)ccc4-c4ccccc4)c4ccccc34)c2)cc1 ; c1ccc(-c2cccc(-c3sc(-c4ccc(-n5c6ccccc6c6ccccc65)cc4-c4ccccc4)c4ccccc34)c2)cc1]

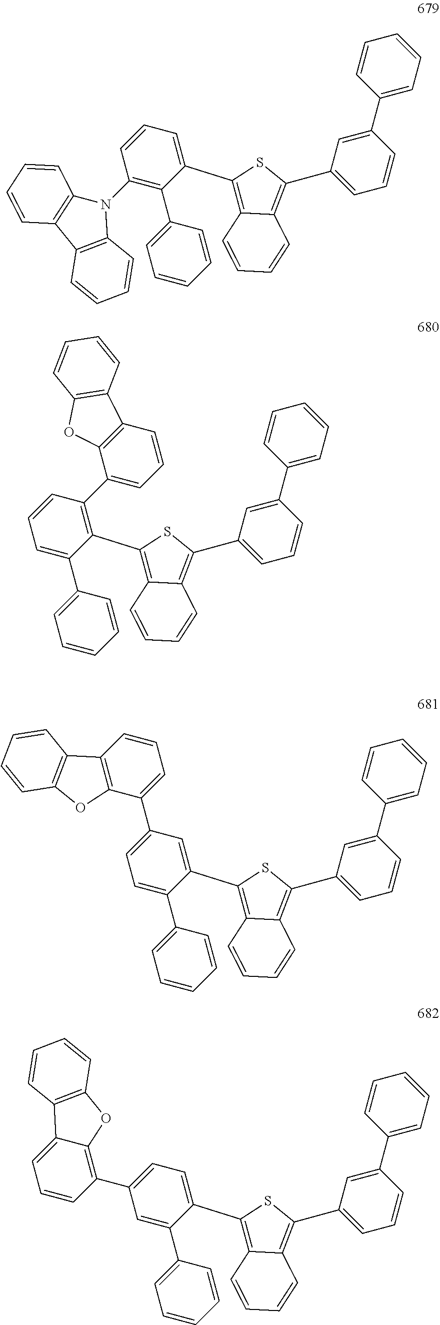

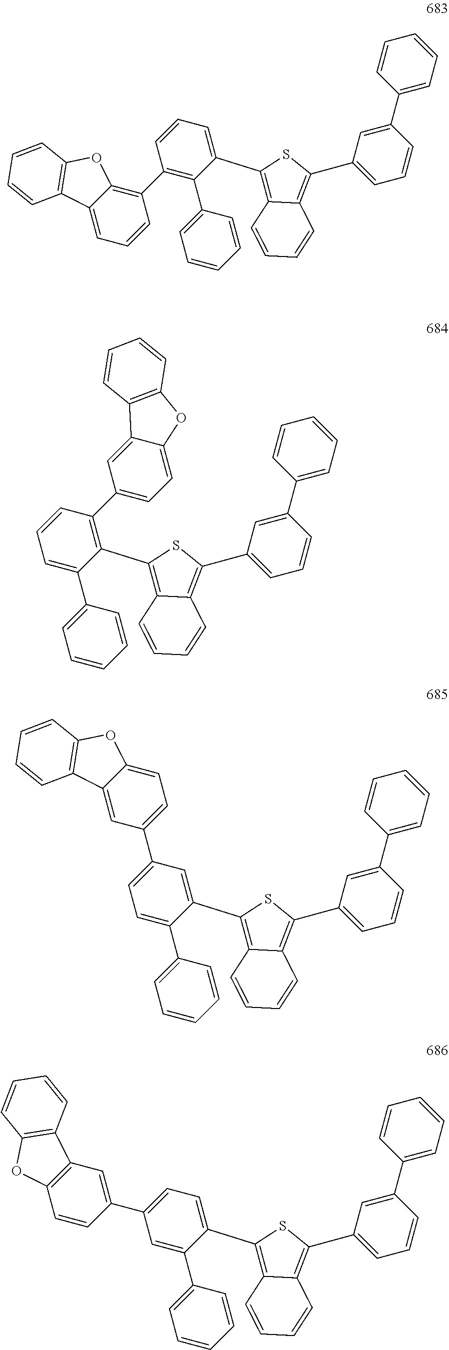 [SMILES: c1ccc(-c2cccc(-c3sc(-c4cccc(-c5cccc6c5oc5ccccc56)c4-c4ccccc4)c4ccccc34)c2)cc1 ; c1ccc(-c2cccc(-c3sc(-c4c(-c5ccccc5)cccc4-c4ccc5oc6ccccc6c5c4)c4ccccc34)c2)cc1 ; c1ccc(-c2cccc(-c3sc(-c4cc(-c5ccc6oc7ccccc7c6c5)ccc4-c4ccccc4)c4ccccc34)c2)cc1 ; c1ccc(-c2cccc(-c3sc(-c4ccc(-c5ccc6oc7ccccc7c6c5)cc4-c4ccccc4)c4ccccc34)c2)cc1]

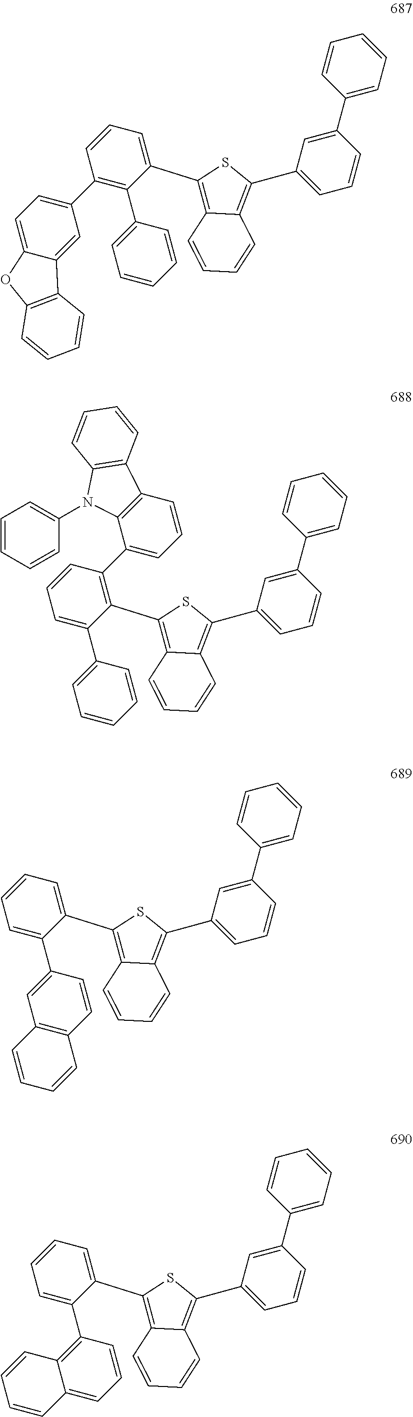 [SMILES: c1ccc(-c2cccc(-c3sc(-c4cccc(-c5ccc6oc7ccccc7c6c5)c4-c4ccccc4)c4ccccc34)c2)cc1 ; c1ccc(-c2cccc(-c3sc(-c4c(-c5ccccc5)cccc4-c4cccc5c6ccccc6n(-c6ccccc6)c45)c4ccccc34)c2)cc1 ; c1ccc(-c2cccc(-c3sc(-c4ccccc4-c4ccc5ccccc5c4)c4ccccc34)c2)cc1 ; c1ccc(-c2cccc(-c3sc(-c4ccccc4-c4cccc5ccccc45)c4ccccc34)c2)cc1]

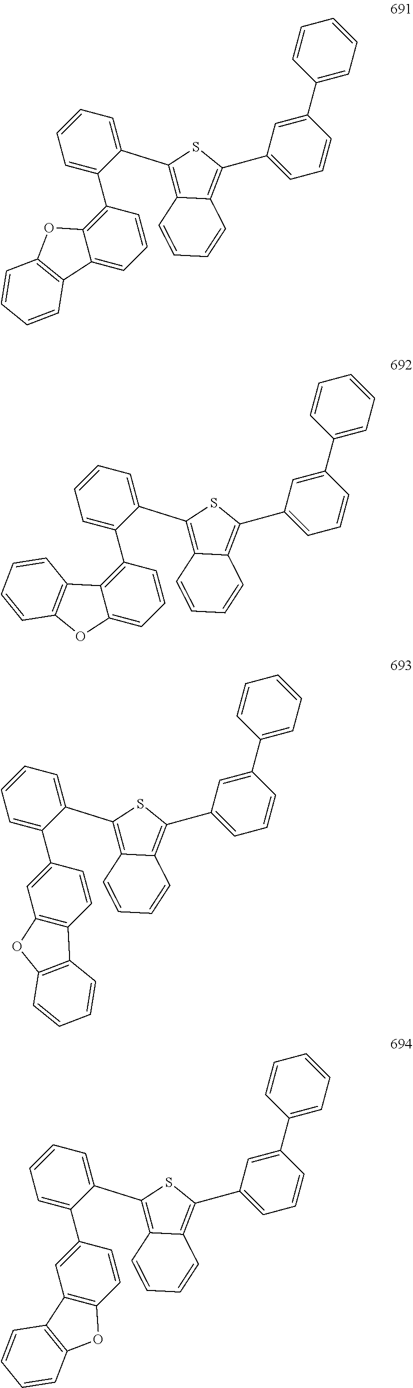 [SMILES: c1ccc(-c2cccc(-c3sc(-c4ccccc4-c4cccc5c4oc4ccccc45)c4ccccc34)c2)cc1 ; c1ccc(-c2cccc(-c3sc(-c4ccccc4-c4cccc5oc6ccccc6c45)c4ccccc34)c2)cc1 ; c1ccc(-c2cccc(-c3sc(-c4ccccc4-c4ccc5c(c4)oc4ccccc45)c4ccccc34)c2)cc1 ; c1ccc(-c2cccc(-c3sc(-c4ccccc4-c4ccc5oc6ccccc6c5c4)c4ccccc34)c2)cc1]

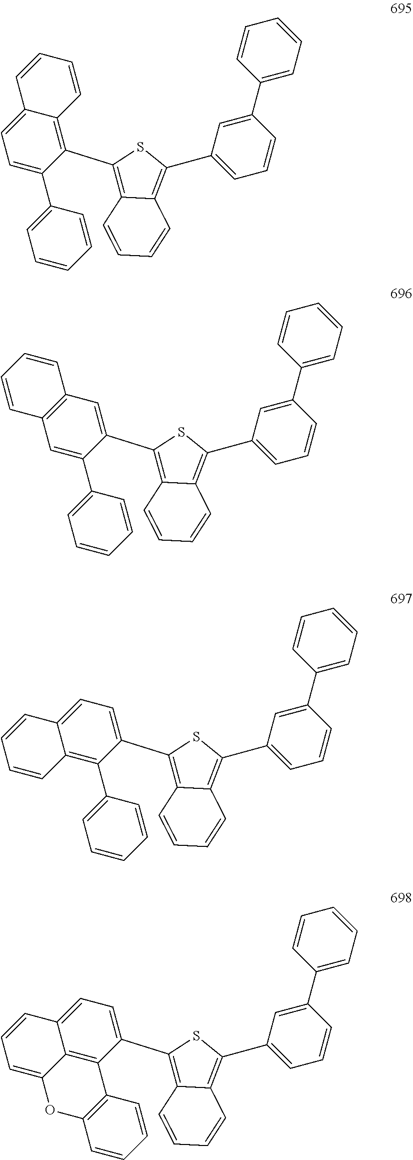 [SMILES: c1ccc(-c2cccc(-c3sc(-c4c(-c5ccccc5)ccc5ccccc45)c4ccccc34)c2)cc1 ; c1ccc(-c2cccc(-c3sc(-c4cc5ccccc5cc4-c4ccccc4)c4ccccc34)c2)cc1 ; c1ccc(-c2cccc(-c3sc(-c4ccc5ccccc5c4-c4ccccc4)c4ccccc34)c2)cc1 ; c1ccc(-c2cccc(-c3sc(-c4ccc5cccc6c5c4-c4ccccc4O6)c4ccccc34)c2)cc1]

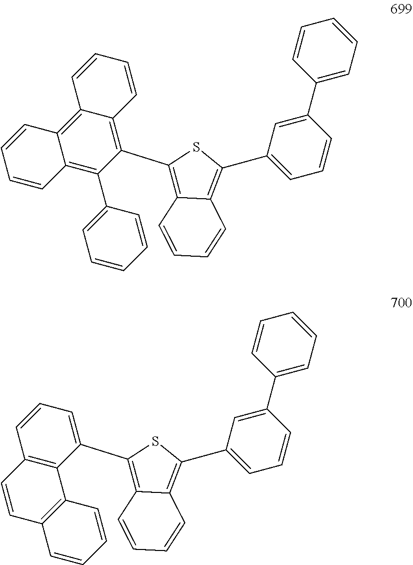 [SMILES: c1ccc(-c2cccc(-c3sc(-c4c(-c5ccccc5)c5ccccc5c5ccccc45)c4ccccc34)c2)cc1 ; c1ccc(-c2cccc(-c3sc(-c4cccc5ccc6ccccc6c45)c4ccccc34)c2)cc1]

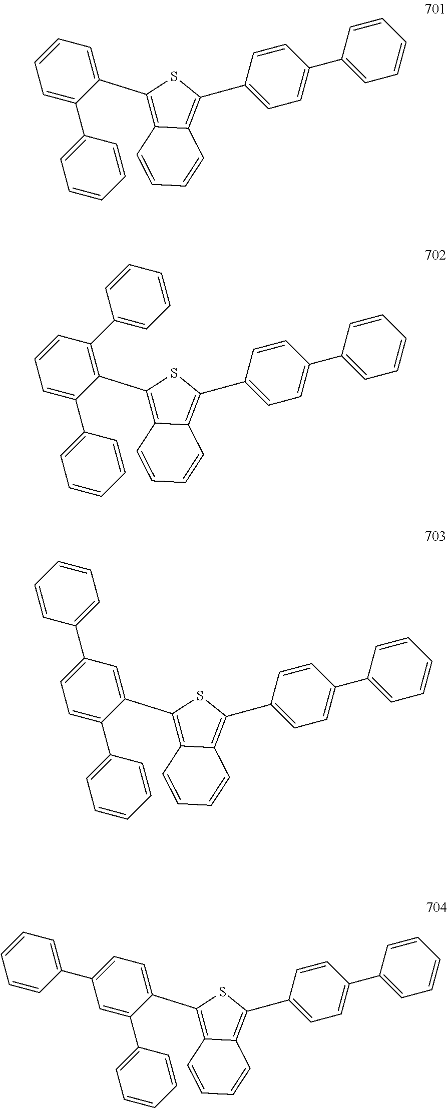 [SMILES: c1ccc(-c2ccc(-c3sc(-c4ccccc4-c4ccccc4)c4ccccc34)cc2)cc1 ; c1ccc(-c2ccc(-c3sc(-c4c(-c5ccccc5)cccc4-c4ccccc4)c4ccccc34)cc2)cc1 ; c1ccc(-c2ccc(-c3sc(-c4cc(-c5ccccc5)ccc4-c4ccccc4)c4ccccc34)cc2)cc1 ; c1ccc(-c2ccc(-c3sc(-c4ccc(-c5ccccc5)cc4-c4ccccc4)c4ccccc34)cc2)cc1]

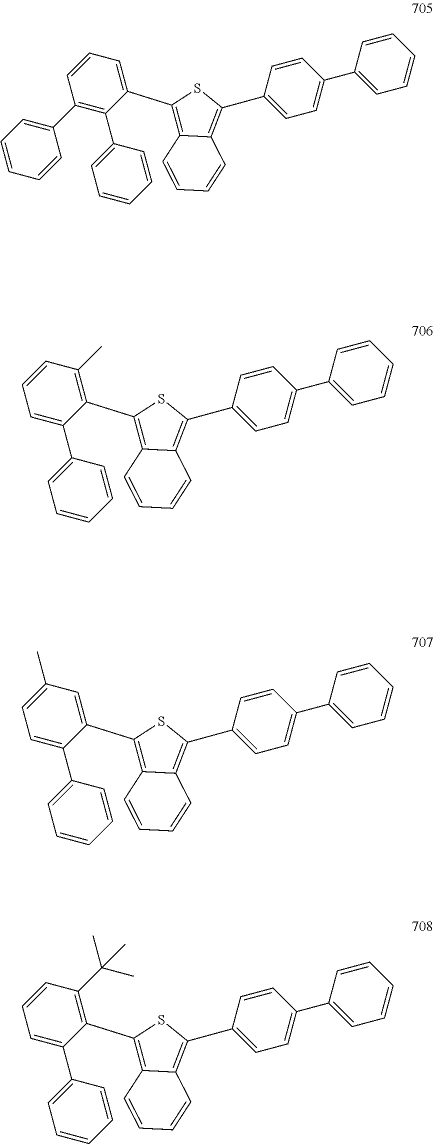 [SMILES: c1ccc(-c2ccc(-c3sc(-c4cccc(-c5ccccc5)c4-c4ccccc4)c4ccccc34)cc2)cc1 ; Cc1cccc(-c2ccccc2)c1-c1sc(-c2ccc(-c3ccccc3)cc2)c2ccccc12 ; Cc1ccc(-c2ccccc2)c(-c2sc(-c3ccc(-c4ccccc4)cc3)c3ccccc23)c1 ; CC(C)(C)c1cccc(-c2ccccc2)c1-c1sc(-c2ccc(-c3ccccc3)cc2)c2ccccc12]

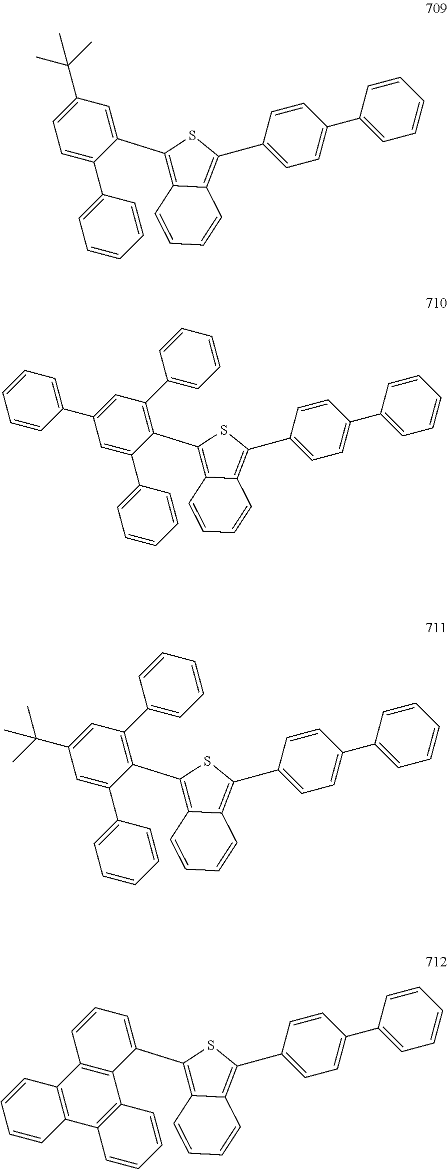 [SMILES: CC(C)(C)c1ccc(-c2ccccc2)c(-c2sc(-c3ccc(-c4ccccc4)cc3)c3ccccc23)c1 ; c1ccc(-c2ccc(-c3sc(-c4c(-c5ccccc5)cc(-c5ccccc5)cc4-c4ccccc4)c4ccccc34)cc2)cc1 ; CC(C)(C)c1cc(-c2ccccc2)c(-c2sc(-c3ccc(-c4ccccc4)cc3)c3ccccc23)c(-c2ccccc2)c1 ; c1ccc(-c2ccc(-c3sc(-c4cccc5c6ccccc6c6ccccc6c45)c4ccccc34)cc2)cc1]

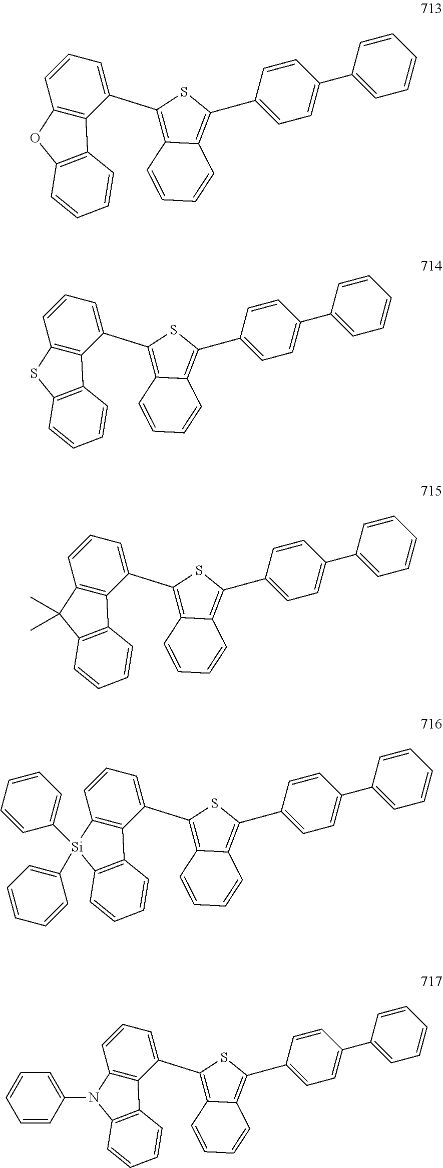 [SMILES: c1ccc(-c2ccc(-c3sc(-c4cccc5oc6ccccc6c45)c4ccccc34)cc2)cc1 ; c1ccc(-c2ccc(-c3sc(-c4cccc5sc6ccccc6c45)c4ccccc34)cc2)cc1 ; CC1(C)c2ccccc2-c2c(-c3sc(-c4ccc(-c5ccccc5)cc4)c4ccccc34)cccc21 ; c1ccc(-c2ccc(-c3sc(-c4cccc5c4-c4ccccc4[Si]5(c4ccccc4)c4ccccc4)c4ccccc34)cc2)cc1 ; c1ccc(-c2ccc(-c3sc(-c4cccc5c4c4ccccc4n5-c4ccccc4)c4ccccc34)cc2)cc1]

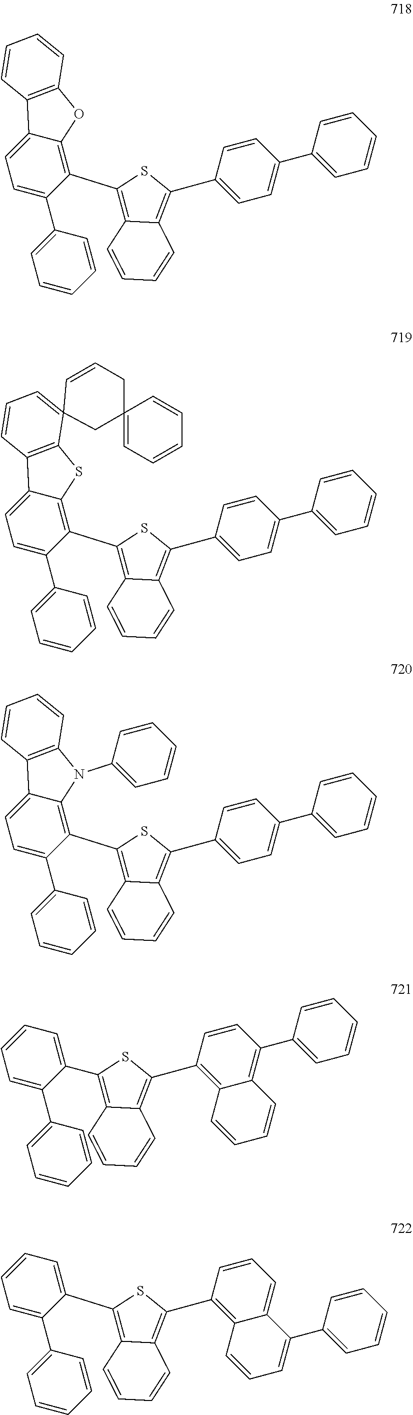

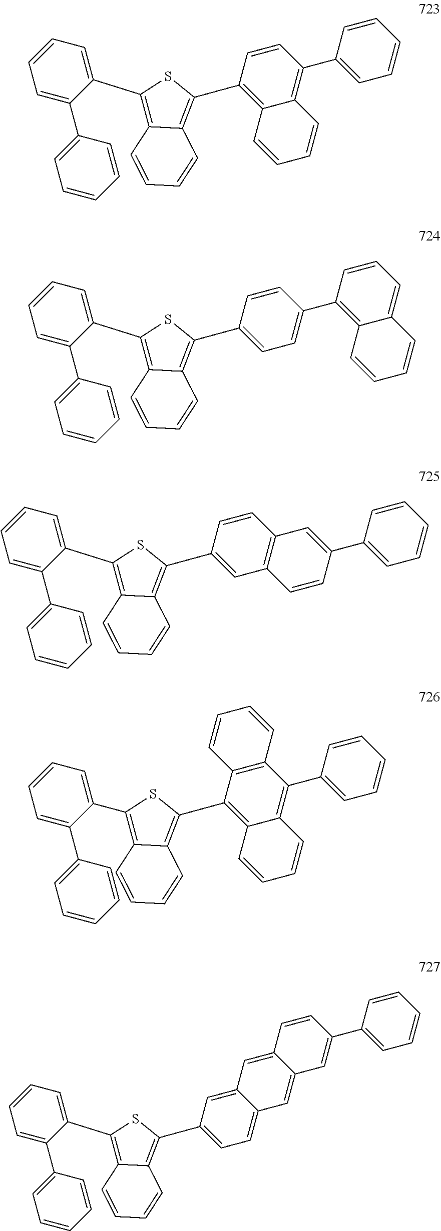 [SMILES: c1ccc(-c2ccccc2-c2sc(-c3ccc(-c4ccccc4)c4ccccc34)c3ccccc23)cc1 ; c1ccc(-c2ccccc2-c2sc(-c3ccc(-c4cccc5ccccc45)cc3)c3ccccc23)cc1 ; c1ccc(-c2ccc3cc(-c4sc(-c5ccccc5-c5ccccc5)c5ccccc45)ccc3c2)cc1 ; c1ccc(-c2ccccc2-c2sc(-c3c4ccccc4c(-c4ccccc4)c4ccccc34)c3ccccc23)cc1 ; c1ccc(-c2ccc3cc4cc(-c5sc(-c6ccccc6-c6ccccc6)c6ccccc56)ccc4cc3c2)cc1]

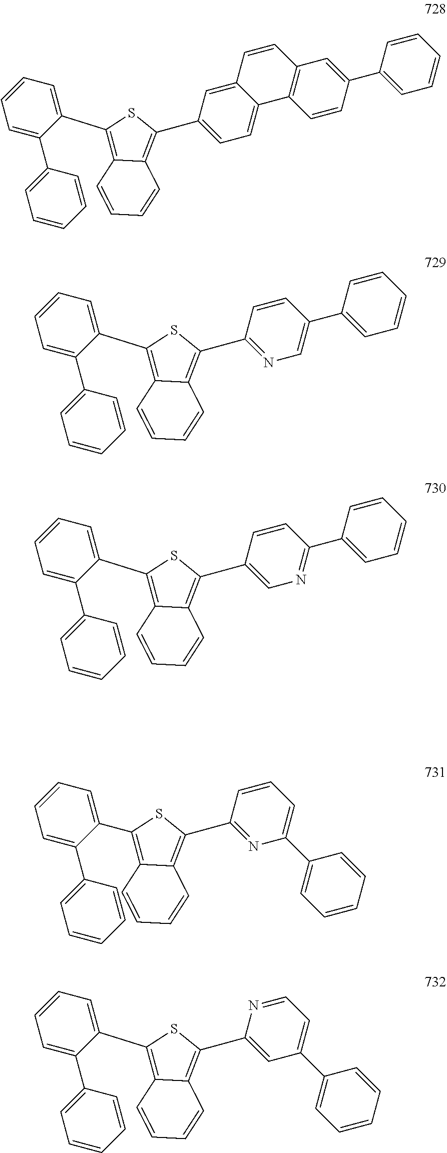 [SMILES: c1ccc(-c2ccc3c(ccc4cc(-c5sc(-c6ccccc6-c6ccccc6)c6ccccc56)ccc43)c2)cc1 ; c1ccc(-c2ccc(-c3sc(-c4ccccc4-c4ccccc4)c4ccccc34)nc2)cc1 ; c1ccc(-c2ccc(-c3sc(-c4ccccc4-c4ccccc4)c4ccccc34)cn2)cc1 ; c1ccc(-c2cccc(-c3sc(-c4ccccc4-c4ccccc4)c4ccccc34)n2)cc1 ; c1ccc(-c2ccnc(-c3sc(-c4ccccc4-c4ccccc4)c4ccccc34)c2)cc1]

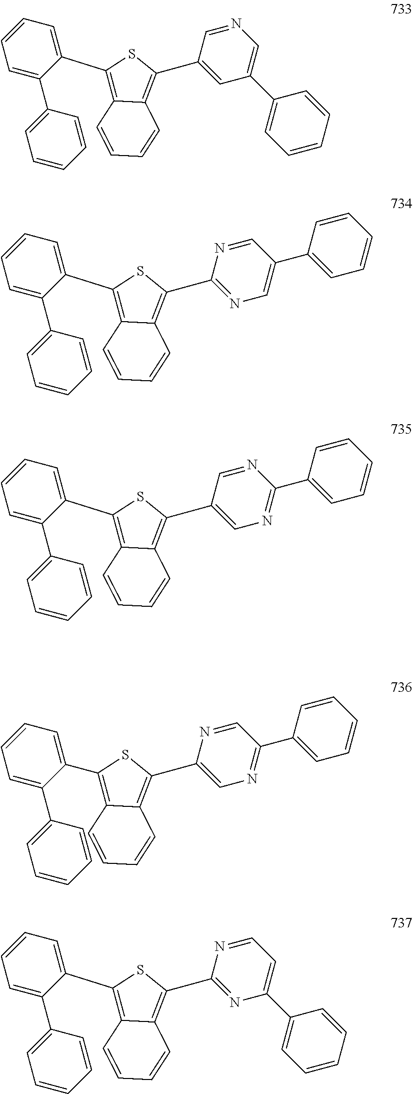 [SMILES: c1ccc(-c2cncc(-c3sc(-c4ccccc4-c4ccccc4)c4ccccc34)c2)cc1 ; c1ccc(-c2cnc(-c3sc(-c4ccccc4-c4ccccc4)c4ccccc34)nc2)cc1 ; c1ccc(-c2ncc(-c3sc(-c4ccccc4-c4ccccc4)c4ccccc34)cn2)cc1 ; c1ccc(-c2cnc(-c3sc(-c4ccccc4-c4ccccc4)c4ccccc34)cn2)cc1 ; c1ccc(-c2ccnc(-c3sc(-c4ccccc4-c4ccccc4)c4ccccc34)n2)cc1]

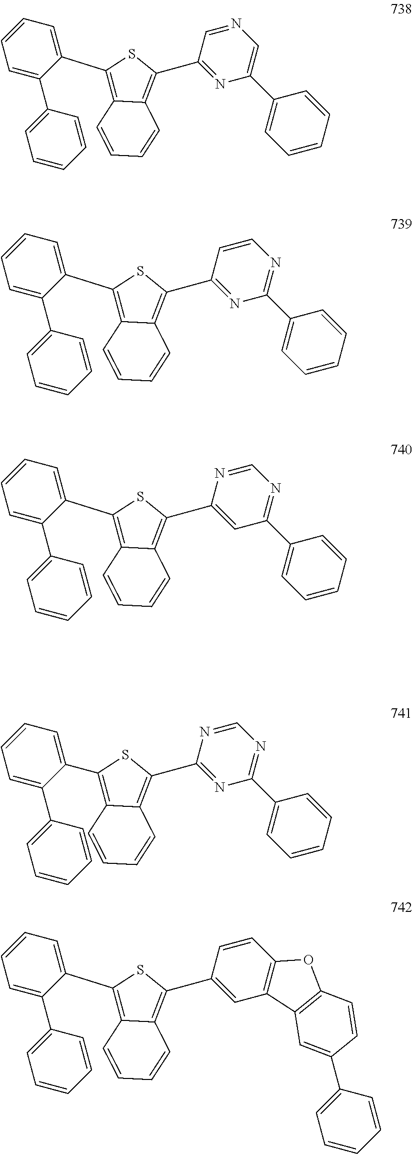 [SMILES: c1ccc(-c2cncc(-c3sc(-c4ccccc4-c4ccccc4)c4ccccc34)n2)cc1 ; c1ccc(-c2nccc(-c3sc(-c4ccccc4-c4ccccc4)c4ccccc34)n2)cc1 ; c1ccc(-c2cc(-c3sc(-c4ccccc4-c4ccccc4)c4ccccc34)ncn2)cc1 ; c1ccc(-c2ncnc(-c3sc(-c4ccccc4-c4ccccc4)c4ccccc34)n2)cc1 ; c1ccc(-c2ccc3oc4ccc(-c5sc(-c6ccccc6-c6ccccc6)c6ccccc56)cc4c3c2)cc1]

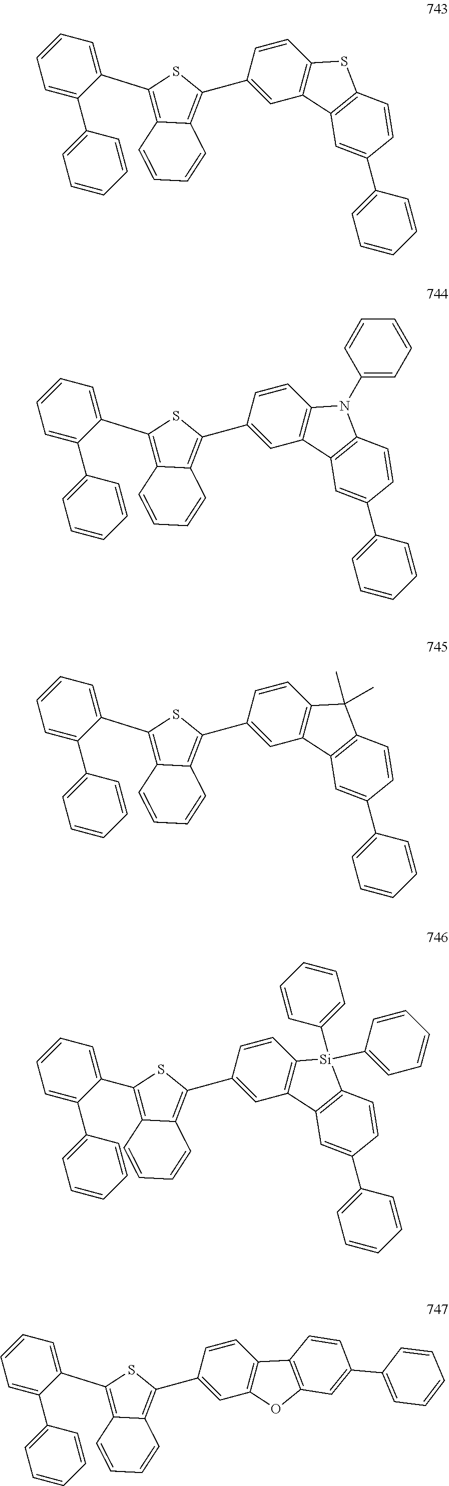 [SMILES: c1ccc(-c2ccc3sc4ccc(-c5sc(-c6ccccc6-c6ccccc6)c6ccccc56)cc4c3c2)cc1 ; c1ccc(-c2ccc3c(c2)c2cc(-c4sc(-c5ccccc5-c5ccccc5)c5ccccc45)ccc2n3-c2ccccc2)cc1 ; CC1(C)c2ccc(-c3ccccc3)cc2-c2cc(-c3sc(-c4ccccc4-c4ccccc4)c4ccccc34)ccc21 ; c1ccc(-c2ccc3c(c2)-c2cc(-c4sc(-c5ccccc5-c5ccccc5)c5ccccc45)ccc2[Si]3(c2ccccc2)c2ccccc2)cc1 ; c1ccc(-c2ccc3c(c2)oc2cc(-c4sc(-c5ccccc5-c5ccccc5)c5ccccc45)ccc23)cc1]

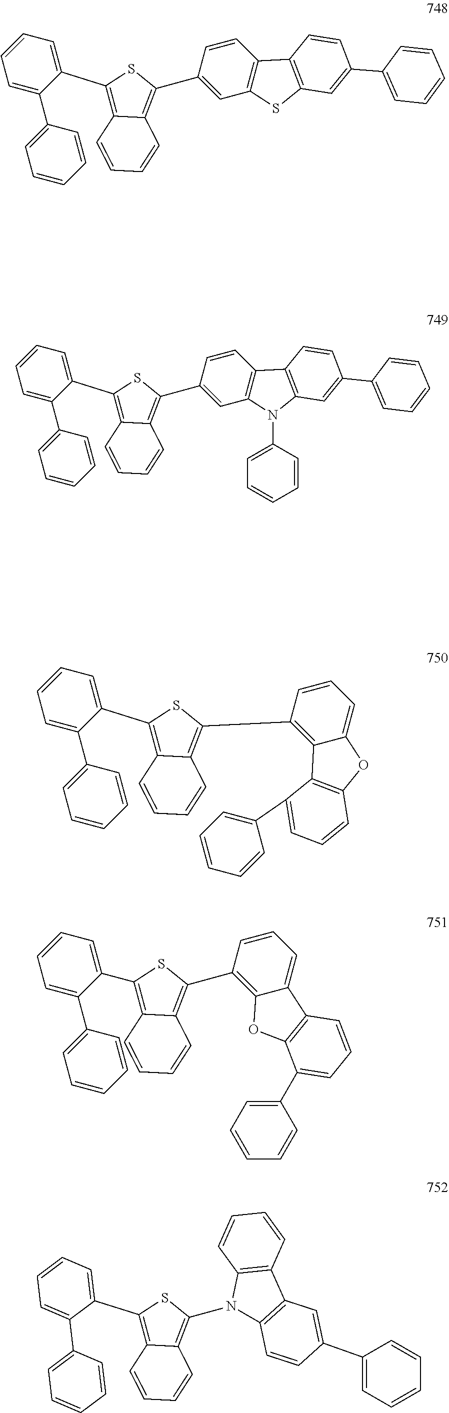 [SMILES: c1ccc(-c2ccc3c(c2)sc2cc(-c4sc(-c5ccccc5-c5ccccc5)c5ccccc45)ccc23)cc1 ; c1ccc(-c2ccc3c4ccc(-c5sc(-c6ccccc6-c6ccccc6)c6ccccc56)cc4n(-c4ccccc4)c3c2)cc1 ; c1ccc(-c2ccccc2-c2sc(-c3cccc4oc5cccc(-c6ccccc6)c5c34)c3ccccc23)cc1 ; c1ccc(-c2ccccc2-c2sc(-c3cccc4c3oc3c(-c5ccccc5)cccc34)c3ccccc23)cc1 ; c1ccc(-c2ccc3c(c2)c2ccccc2n3-c2sc(-c3ccccc3-c3ccccc3)c3ccccc23)cc1]

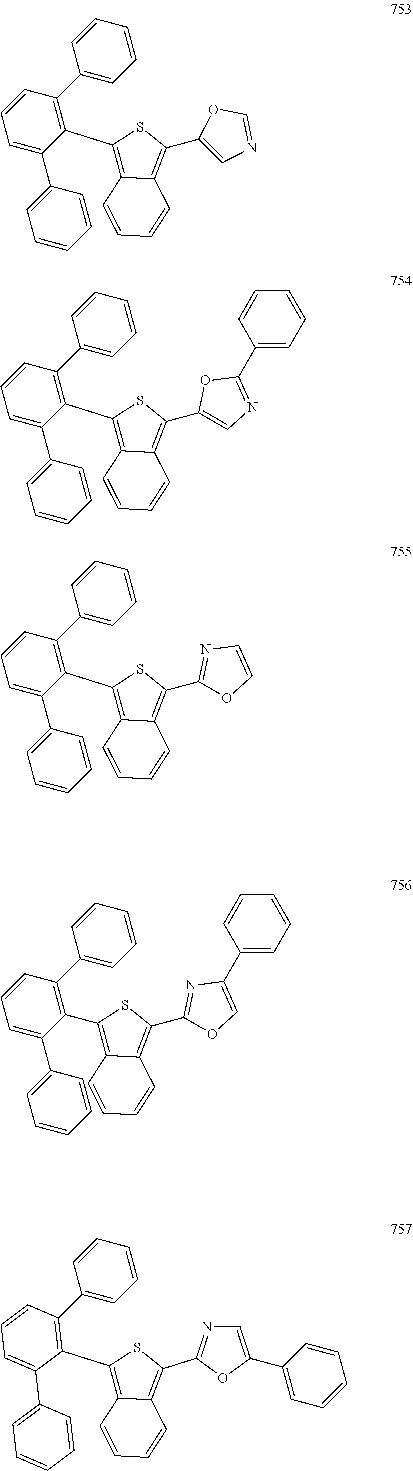

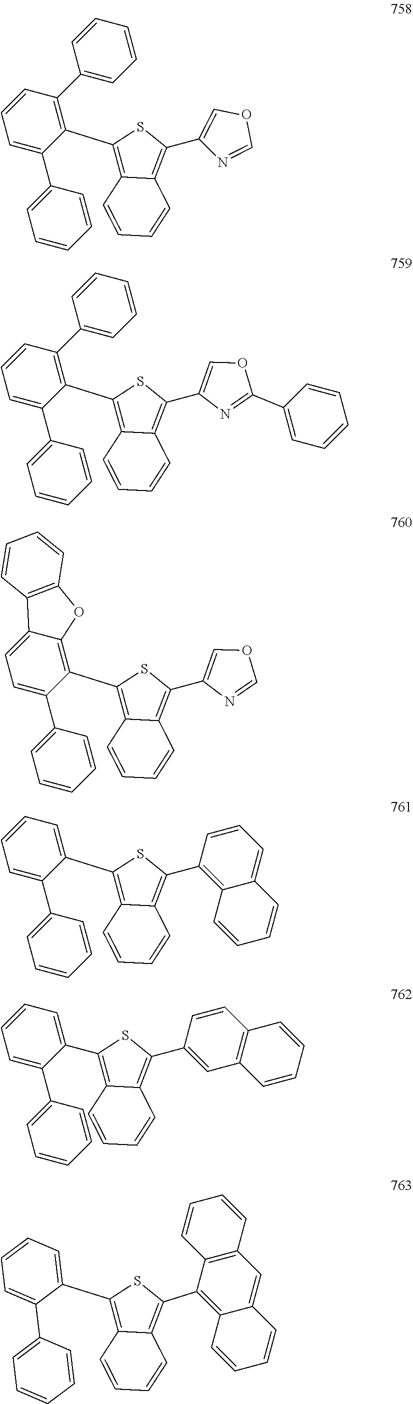

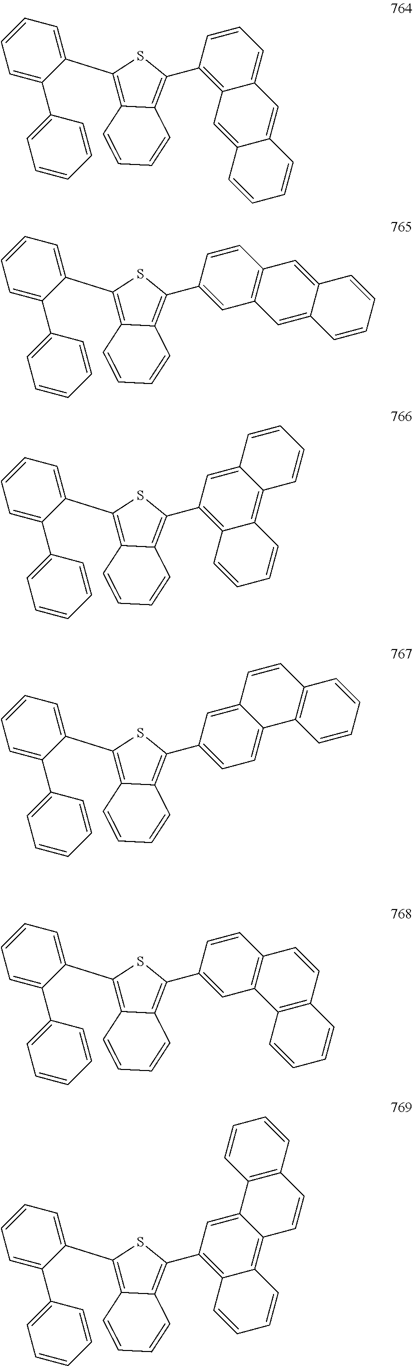

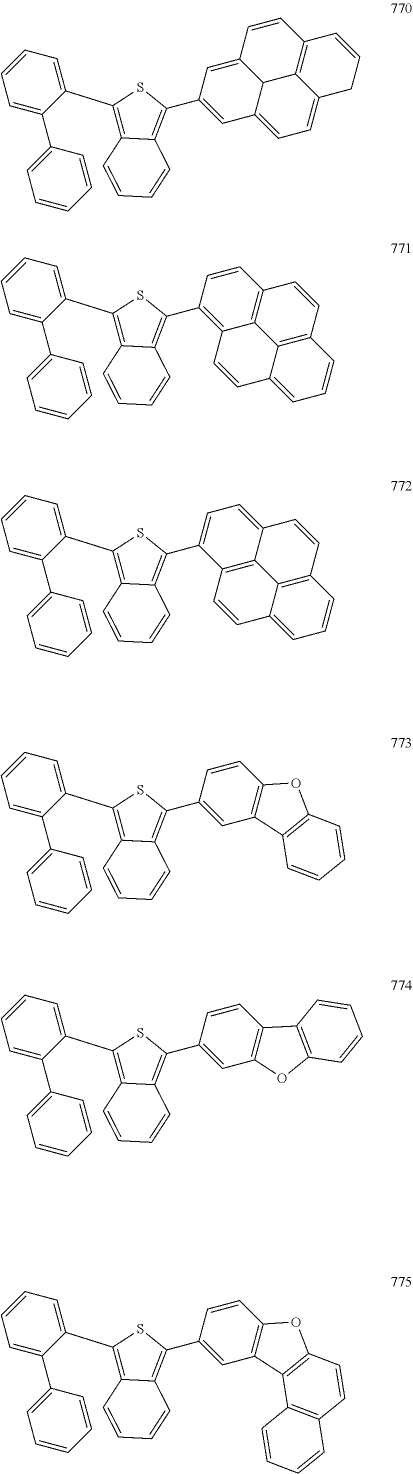 [SMILES: C1=CC2=C3C(=CC=C4C=C(c5sc(-c6ccccc6-c6ccccc6)c6ccccc56)C=C(C=C2)C43)C1 ; c1ccc(-c2ccccc2-c2sc(-c3ccc4ccc5cccc6ccc3c4c56)c3ccccc23)cc1 ; c1ccc(-c2ccccc2-c2sc(-c3ccc4ccc5cccc6ccc3c4c56)c3ccccc23)cc1 ; c1ccc(-c2ccccc2-c2sc(-c3ccc4oc5ccccc5c4c3)c3ccccc23)cc1 ; c1ccc(-c2ccccc2-c2sc(-c3ccc4c(c3)oc3ccccc34)c3ccccc23)cc1 ; c1ccc(-c2ccccc2-c2sc(-c3ccc4oc5ccc6ccccc6c5c4c3)c3ccccc23)cc1]

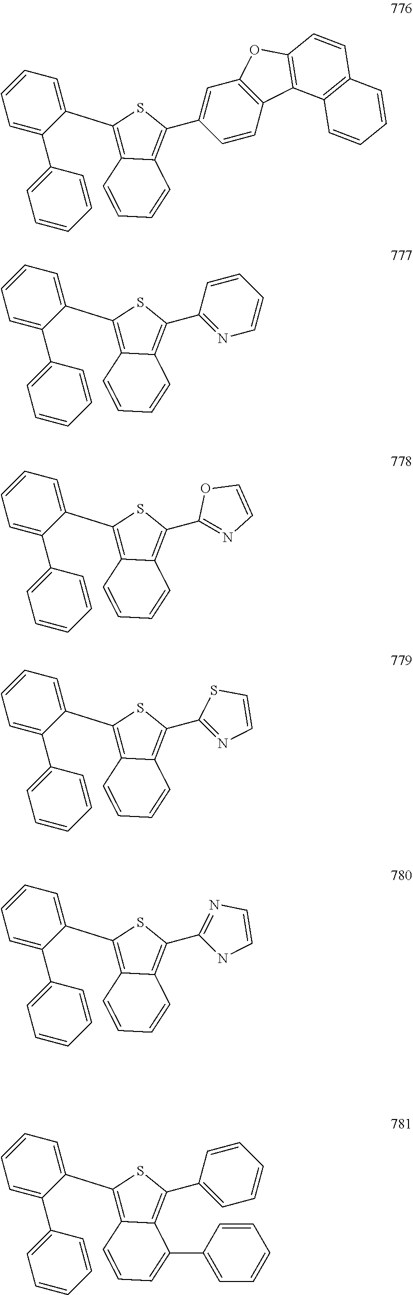

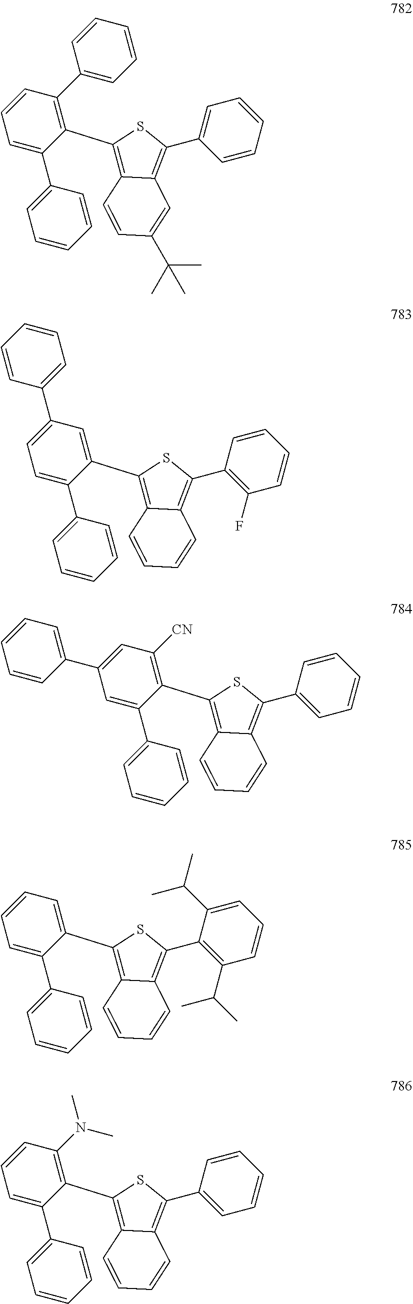

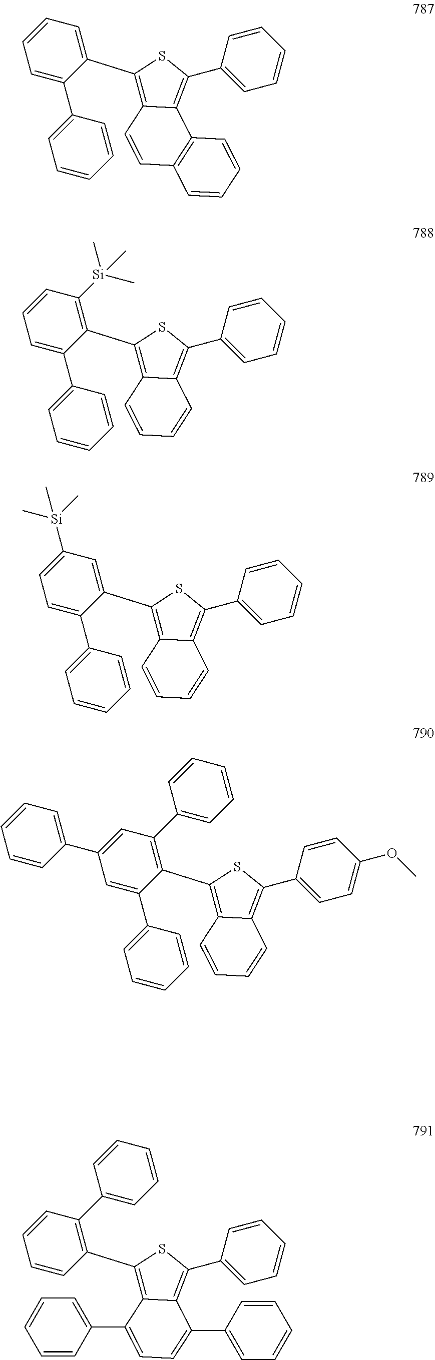

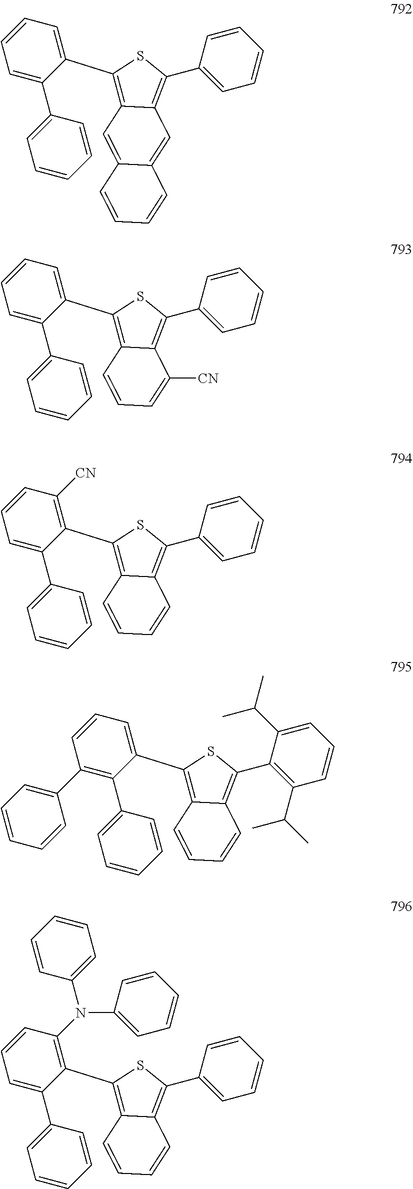 [SMILES: c1ccc(-c2ccccc2-c2sc(-c3ccccc3)c3cc4ccccc4cc23)cc1 ; N#Cc1cccc2c(-c3ccccc3-c3ccccc3)sc(-c3ccccc3)c12 ; N#Cc1cccc(-c2ccccc2)c1-c1sc(-c2ccccc2)c2ccccc12 ; CC(C)c1cccc(C(C)C)c1-c1sc(-c2cccc(-c3ccccc3)c2-c2ccccc2)c2ccccc12 ; c1ccc(-c2cccc(N(c3ccccc3)c3ccccc3)c2-c2sc(-c3ccccc3)c3ccccc23)cc1]

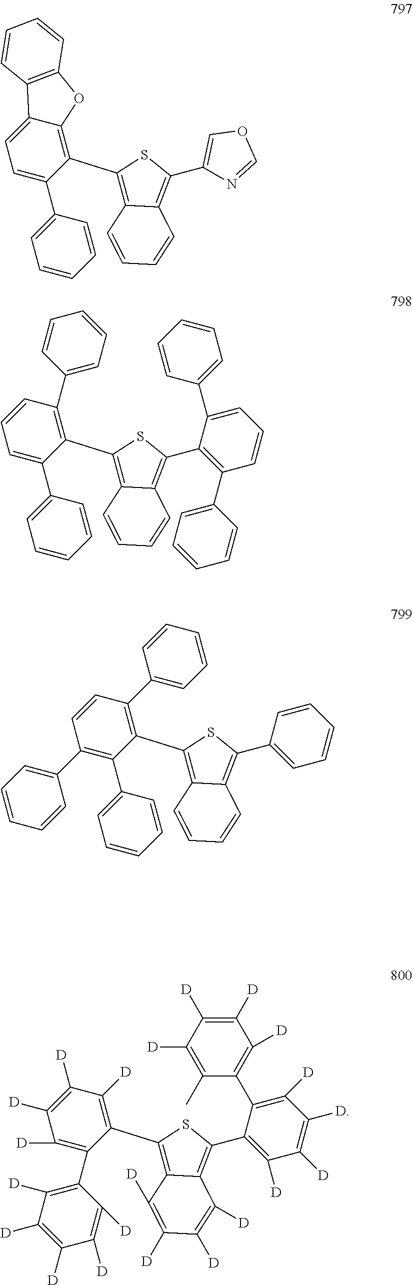

In an embodiment, the fluorescent host may be represented by Formula FH-3:

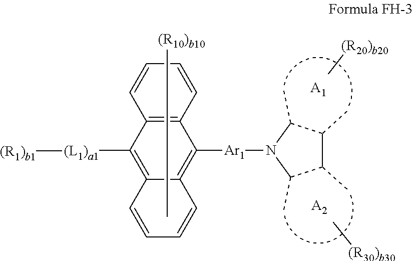

wherein, in Formula FH-3,

Ar₁ is group represented by Formula 2,

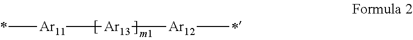

Ar₁ may include at least one cyano group,

A₁ and A₂ may each independently be a C₅-C₃₀ carbocyclic group or a C₁-C₃₀ heterocyclic group,

L₁ may be a substituted or unsubstituted C₅-C₃₀ carbocyclic group or a substituted or unsubstituted C₁-C₃₀ heterocyclic group,

a1 may be 0, 1, 2, or 3,

when a1 is 2 or more, two or more of L₁(s) may be identical to or different from each other,

m1 may be 0, 1, 2, or 3,

Ar₁₁ may be a group represented by Formula 4, Ar₁₂ may be a group represented by Formula 5, and Ar₁₃ may be a group represented by Formula 6,

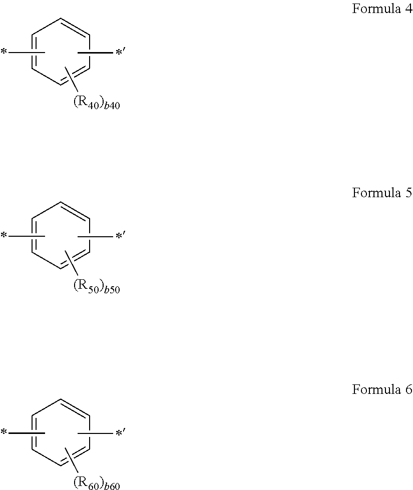

wherein, in the formulae shown above,

R₁, R₁₀, R₂₀, R₃₀, R₄₀, R₅₀, and R₆₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

b1 may be an integer from 1 to 5,

when b1 is 2 or more, two or more of R₁₀(s) may be identical to or different from each other,

b10 may be an integer from 1 to 8,

when b10 is 2 or more, two or more of R₁₀(s) may be identical to or different from each other,

b20 and b30 may each independently be an integer from 1 to 4,

when b20 is 2 or more, two or more of R₂₀ may be identical to or different from each other, and when b30 is 2 or more, two or more of R₃₀ may be identical to or different from each other,

b40, b50, and b60 may each independently be an integer from 1 to 4,

when b40 is 2 or more, two or more of R₄₀(s) may be identical to or different from each other, when b50 is 2 or more, two or more of R₅₀(s) may be identical to or different from each other, and when b60 is 2 or more, two or more of R₆₀(s) may be identical to or different from each other, and

* and *′ each indicate a binding site to an adjacent atom.

In an embodiment, the fluorescent host represented by Formula FH-3 may be Group FH3, but embodiments of the present disclosure are not limited thereto:

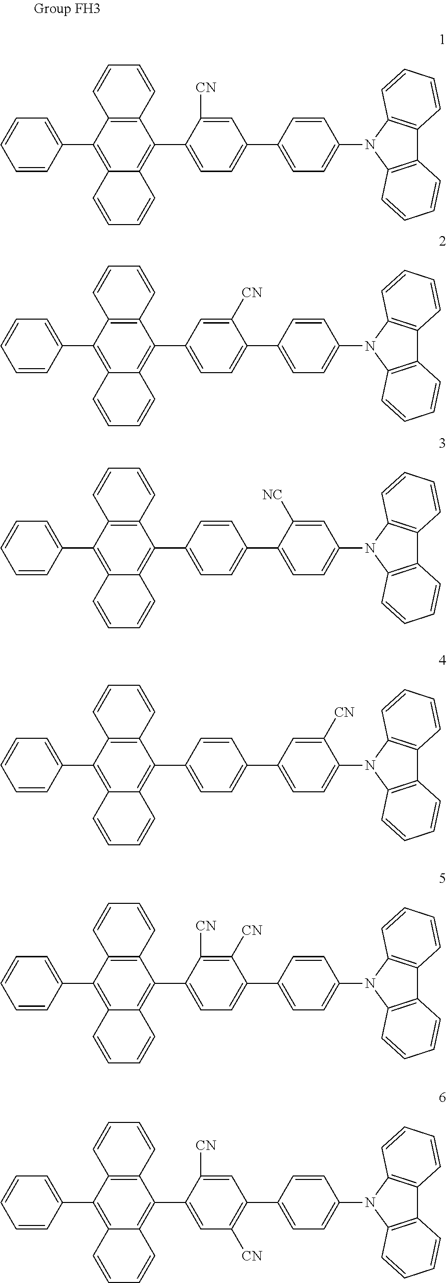

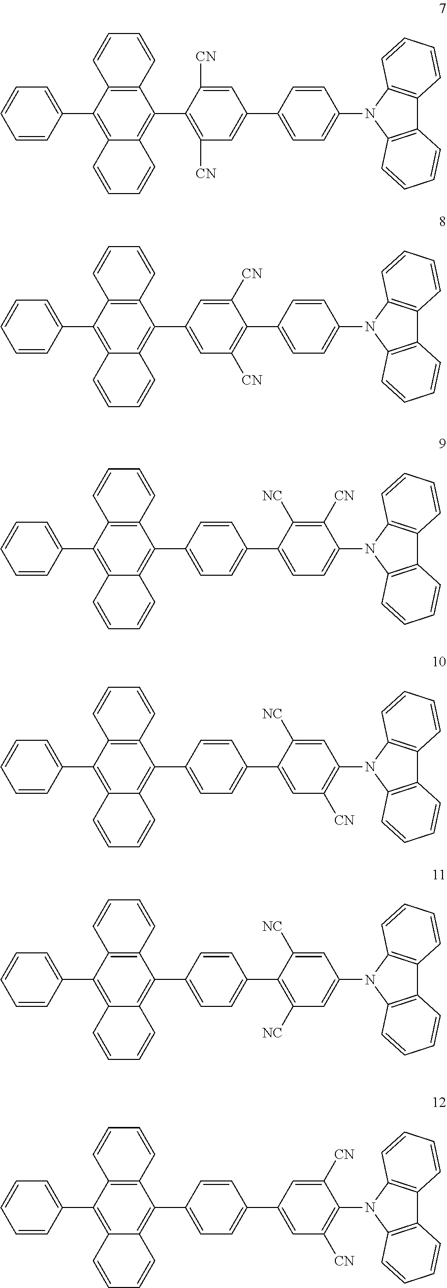

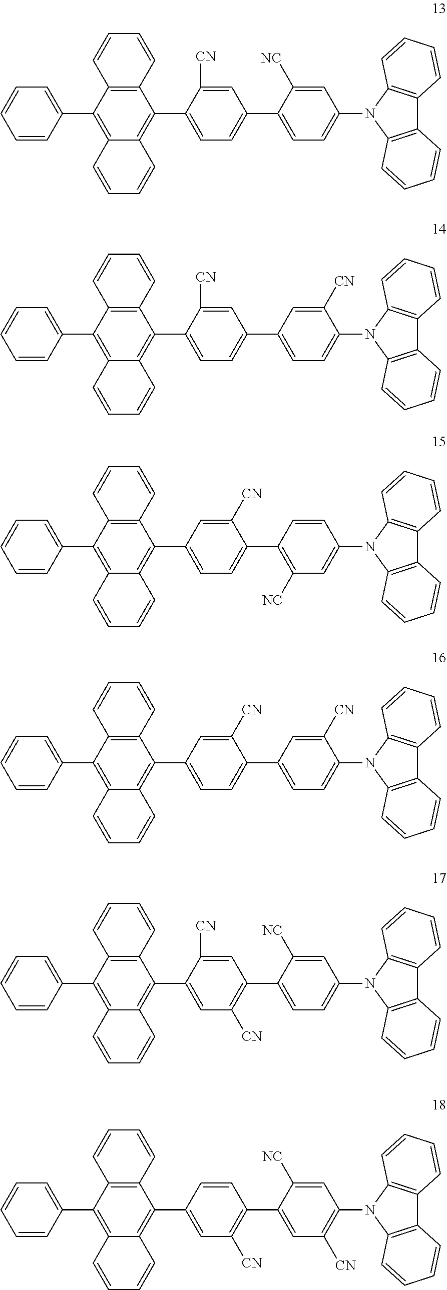

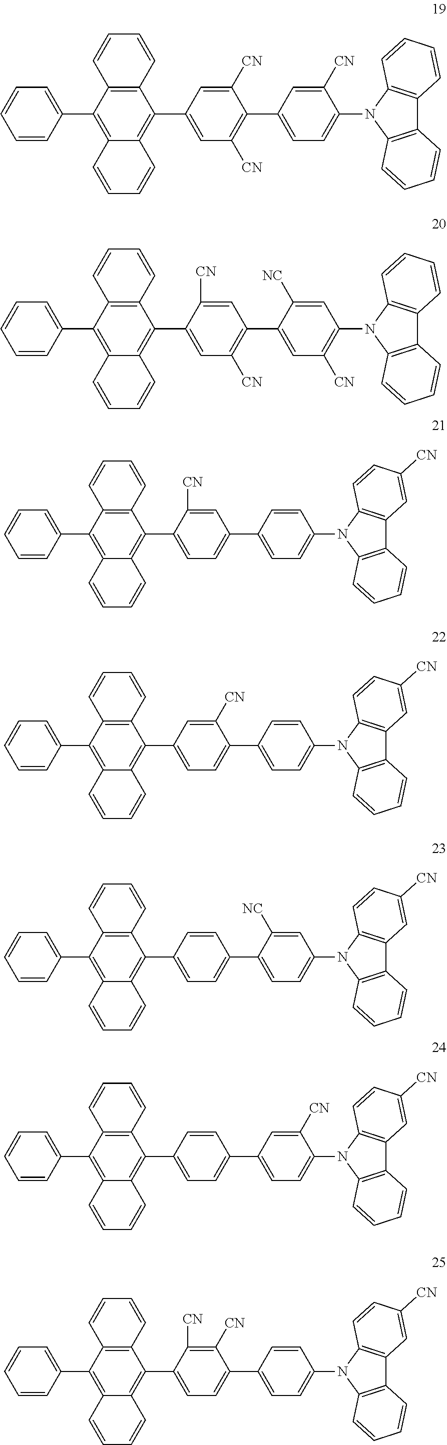

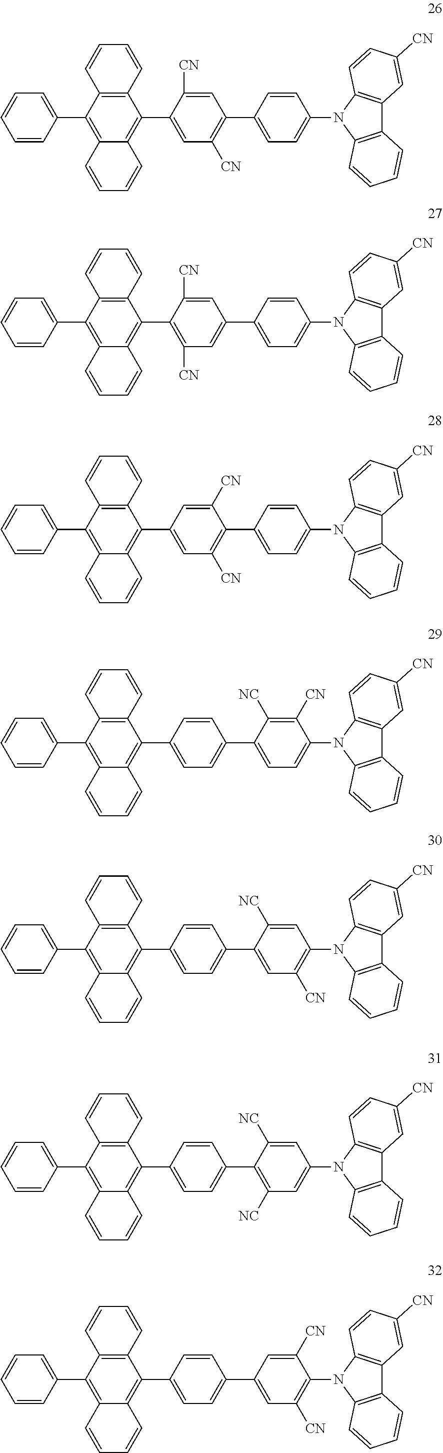

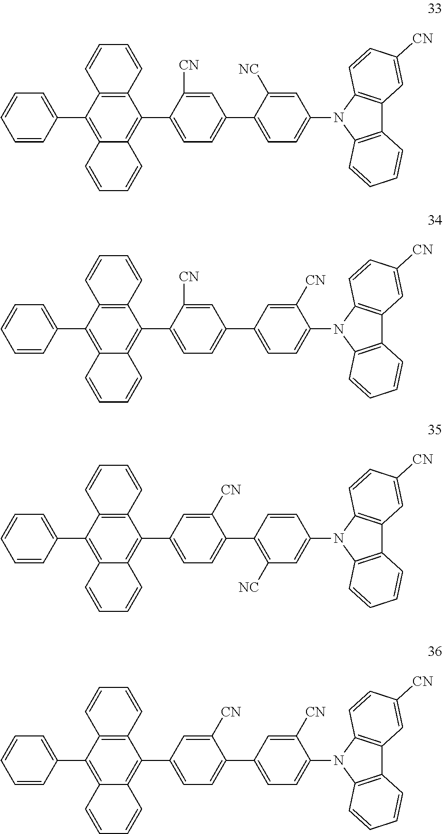 [SMILES: N#Cc1ccc2c(c1)c1ccccc1n2-c1ccc(-c2ccc(-c3c4ccccc4c(-c4ccccc4)c4ccccc34)c(C#N)c2)c(C#N)c1 ; N#Cc1ccc2c(c1)c1ccccc1n2-c1ccc(-c2ccc(-c3c4ccccc4c(-c4ccccc4)c4ccccc34)c(C#N)c2)cc1C#N ; N#Cc1ccc2c(c1)c1ccccc1n2-c1ccc(-c2ccc(-c3c4ccccc4c(-c4ccccc4)c4ccccc34)cc2C#N)c(C#N)c1 ; N#Cc1ccc2c(c1)c1ccccc1n2-c1ccc(-c2ccc(-c3c4ccccc4c(-c4ccccc4)c4ccccc34)cc2C#N)cc1C#N]

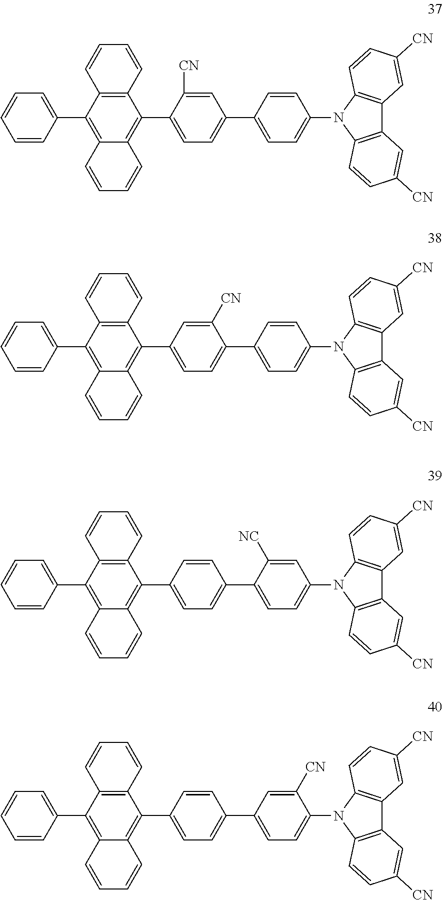 [SMILES: N#Cc1ccc2c(c1)c1cc(C#N)ccc1n2-c1ccc(-c2ccc(-c3c4ccccc4c(-c4ccccc4)c4ccccc34)c(C#N)c2)cc1 ; N#Cc1ccc2c(c1)c1cc(C#N)ccc1n2-c1ccc(-c2ccc(-c3c4ccccc4c(-c4ccccc4)c4ccccc34)cc2C#N)cc1 ; N#Cc1ccc2c(c1)c1cc(C#N)ccc1n2-c1ccc(-c2ccc(-c3c4ccccc4c(-c4ccccc4)c4ccccc34)cc2)c(C#N)c1 ; N#Cc1ccc2c(c1)c1cc(C#N)ccc1n2-c1ccc(-c2ccc(-c3c4ccccc4c(-c4ccccc4)c4ccccc34)cc2)cc1C#N]

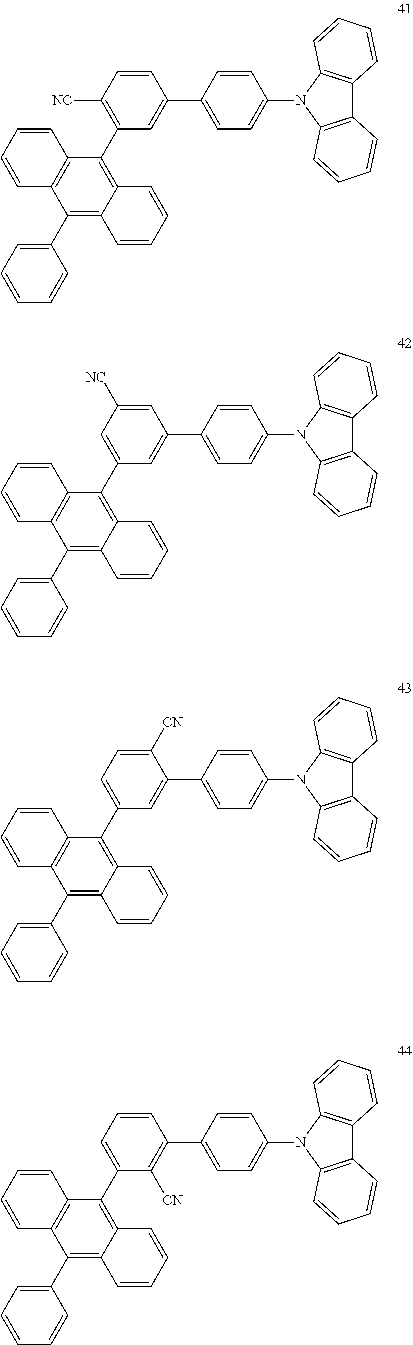 [SMILES: N#Cc1ccc(-c2ccc(-n3c4ccccc4c4ccccc43)cc2)cc1-c1c2ccccc2c(-c2ccccc2)c2ccccc12 ; N#Cc1cc(-c2ccc(-n3c4ccccc4c4ccccc43)cc2)cc(-c2c3ccccc3c(-c3ccccc3)c3ccccc23)c1 ; N#Cc1ccc(-c2c3ccccc3c(-c3ccccc3)c3ccccc23)cc1-c1ccc(-n2c3ccccc3c3ccccc32)cc1 ; N#Cc1c(-c2ccc(-n3c4ccccc4c4ccccc43)cc2)cccc1-c1c2ccccc2c(-c2ccccc2)c2ccccc12]

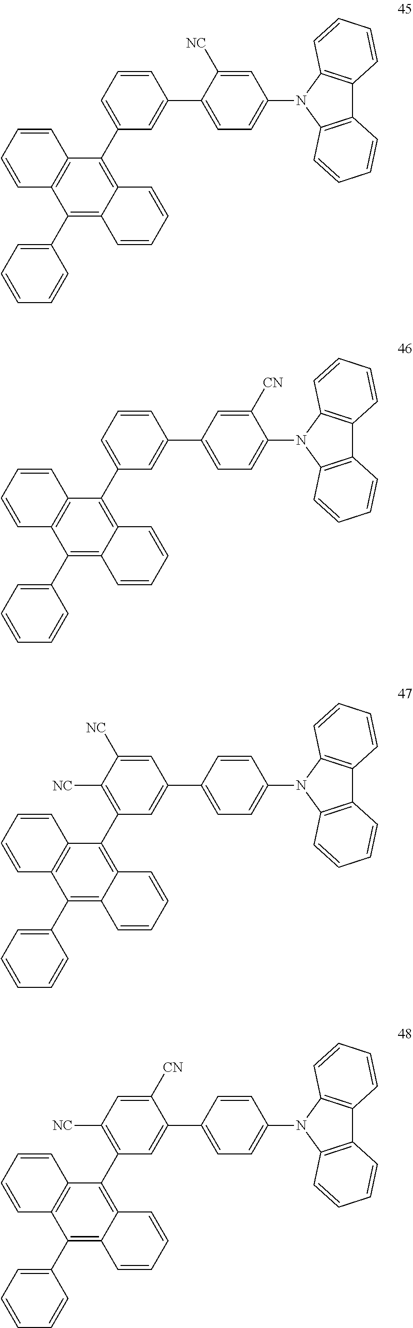 [SMILES: N#Cc1cc(-n2c3ccccc3c3ccccc32)ccc1-c1cccc(-c2c3ccccc3c(-c3ccccc3)c3ccccc23)c1 ; N#Cc1cc(-c2cccc(-c3c4ccccc4c(-c4ccccc4)c4ccccc34)c2)ccc1-n1c2ccccc2c2ccccc21 ; N#Cc1cc(-c2ccc(-n3c4ccccc4c4ccccc43)cc2)cc(-c2c3ccccc3c(-c3ccccc3)c3ccccc23)c1C#N ; N#Cc1cc(C#N)c(-c2c3ccccc3c(-c3ccccc3)c3ccccc23)cc1-c1ccc(-n2c3ccccc3c3ccccc32)cc1]

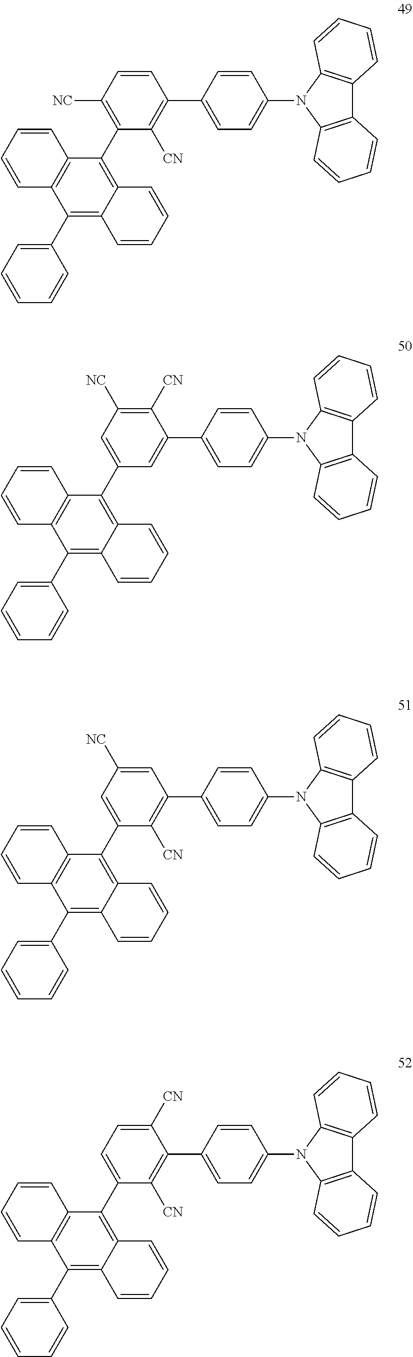 [SMILES: N#Cc1ccc(-c2ccc(-n3c4ccccc4c4ccccc43)cc2)c(C#N)c1-c1c2ccccc2c(-c2ccccc2)c2ccccc12 ; N#Cc1cc(-c2c3ccccc3c(-c3ccccc3)c3ccccc23)cc(-c2ccc(-n3c4ccccc4c4ccccc43)cc2)c1C#N ; N#Cc1cc(-c2ccc(-n3c4ccccc4c4ccccc43)cc2)c(C#N)c(-c2c3ccccc3c(-c3ccccc3)c3ccccc23)c1 ; N#Cc1ccc(-c2c3ccccc3c(-c3ccccc3)c3ccccc23)c(C#N)c1-c1ccc(-n2c3ccccc3c3ccccc32)cc1]

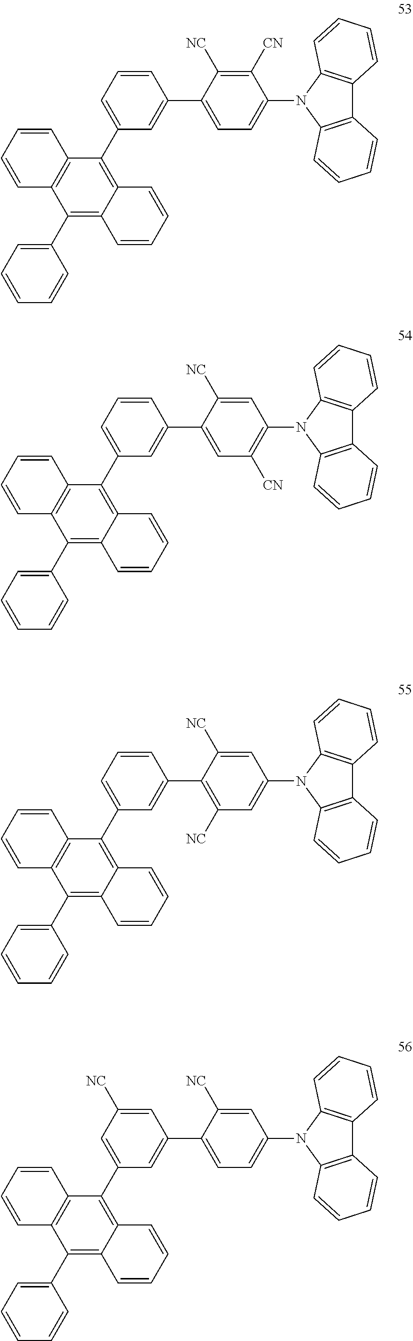 [SMILES: N#Cc1c(-c2cccc(-c3c4ccccc4c(-c4ccccc4)c4ccccc34)c2)ccc(-n2c3ccccc3c3ccccc32)c1C#N ; N#Cc1cc(-n2c3ccccc3c3ccccc32)c(C#N)cc1-c1cccc(-c2c3ccccc3c(-c3ccccc3)c3ccccc23)c1 ; N#Cc1cc(-n2c3ccccc3c3ccccc32)cc(C#N)c1-c1cccc(-c2c3ccccc3c(-c3ccccc3)c3ccccc23)c1 ; N#Cc1cc(-c2ccc(-n3c4ccccc4c4ccccc43)cc2C#N)cc(-c2c3ccccc3c(-c3ccccc3)c3ccccc23)c1]

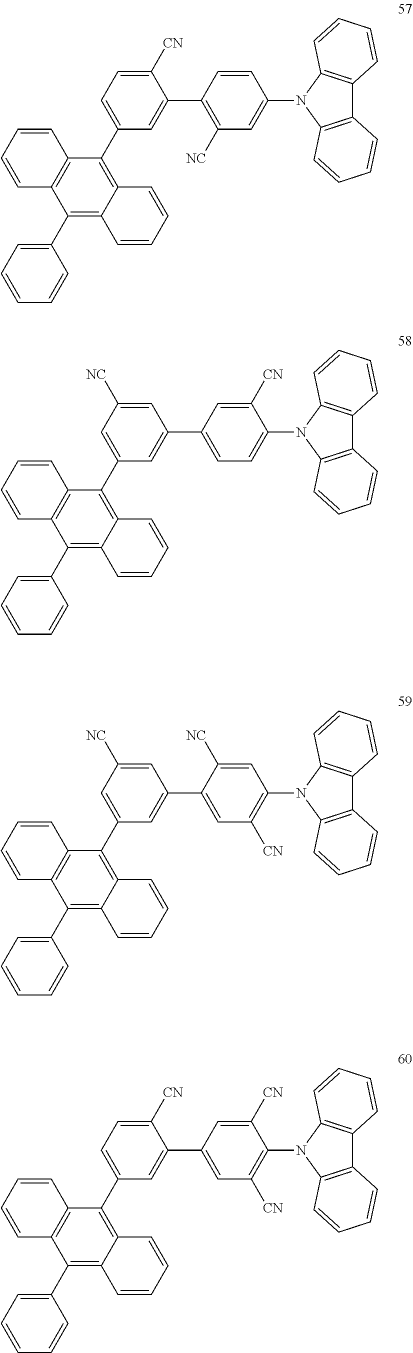 [SMILES: N#Cc1cc(-n2c3ccccc3c3ccccc32)ccc1-c1cc(-c2c3ccccc3c(-c3ccccc3)c3ccccc23)ccc1C#N ; N#Cc1cc(-c2ccc(-n3c4ccccc4c4ccccc43)c(C#N)c2)cc(-c2c3ccccc3c(-c3ccccc3)c3ccccc23)c1 ; N#Cc1cc(-c2cc(C#N)c(-n3c4ccccc4c4ccccc43)cc2C#N)cc(-c2c3ccccc3c(-c3ccccc3)c3ccccc23)c1 ; N#Cc1ccc(-c2c3ccccc3c(-c3ccccc3)c3ccccc23)cc1-c1cc(C#N)c(-n2c3ccccc3c3ccccc32)c(C#N)c1]

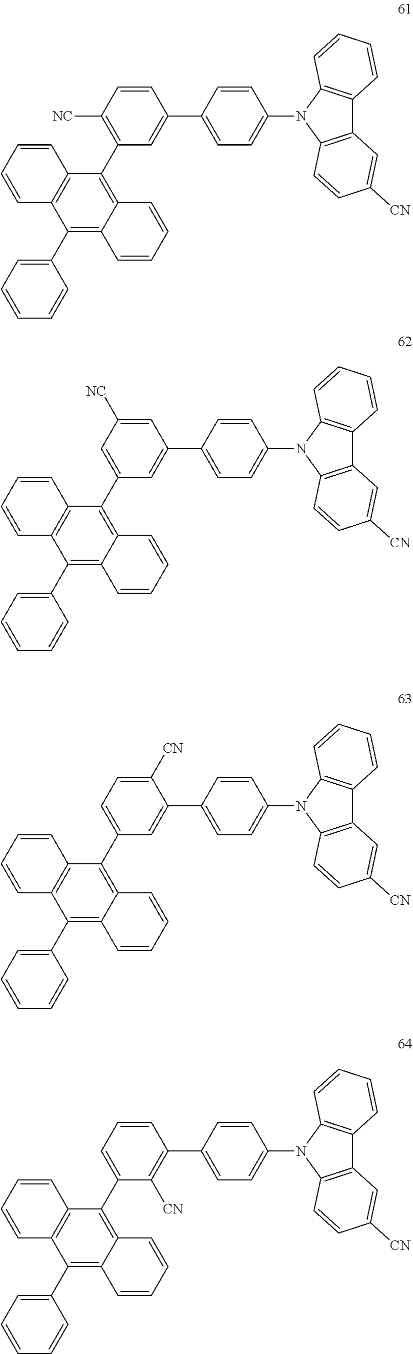 [SMILES: N#Cc1ccc2c(c1)c1ccccc1n2-c1ccc(-c2ccc(C#N)c(-c3c4ccccc4c(-c4ccccc4)c4ccccc34)c2)cc1 ; N#Cc1cc(-c2ccc(-n3c4ccccc4c4cc(C#N)ccc43)cc2)cc(-c2c3ccccc3c(-c3ccccc3)c3ccccc23)c1 ; N#Cc1ccc2c(c1)c1ccccc1n2-c1ccc(-c2cc(-c3c4ccccc4c(-c4ccccc4)c4ccccc34)ccc2C#N)cc1 ; N#Cc1ccc2c(c1)c1ccccc1n2-c1ccc(-c2cccc(-c3c4ccccc4c(-c4ccccc4)c4ccccc34)c2C#N)cc1]

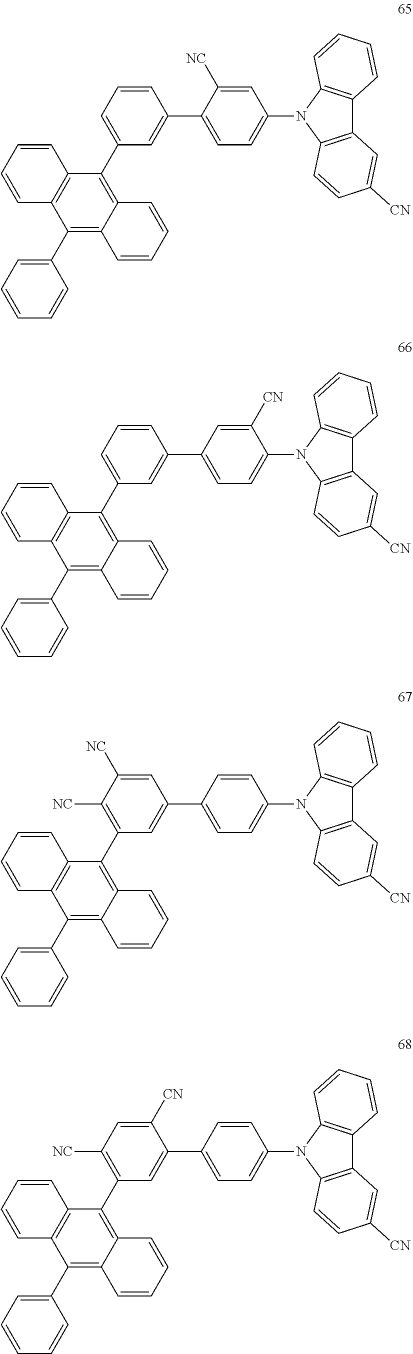 [SMILES: N#Cc1ccc2c(c1)c1ccccc1n2-c1ccc(-c2cccc(-c3c4ccccc4c(-c4ccccc4)c4ccccc34)c2)c(C#N)c1 ; N#Cc1ccc2c(c1)c1ccccc1n2-c1ccc(-c2cccc(-c3c4ccccc4c(-c4ccccc4)c4ccccc34)c2)cc1C#N ; N#Cc1ccc2c(c1)c1ccccc1n2-c1ccc(-c2cc(C#N)c(C#N)c(-c3c4ccccc4c(-c4ccccc4)c4ccccc34)c2)cc1 ; N#Cc1ccc2c(c1)c1ccccc1n2-c1ccc(-c2cc(-c3c4ccccc4c(-c4ccccc4)c4ccccc34)c(C#N)cc2C#N)cc1]

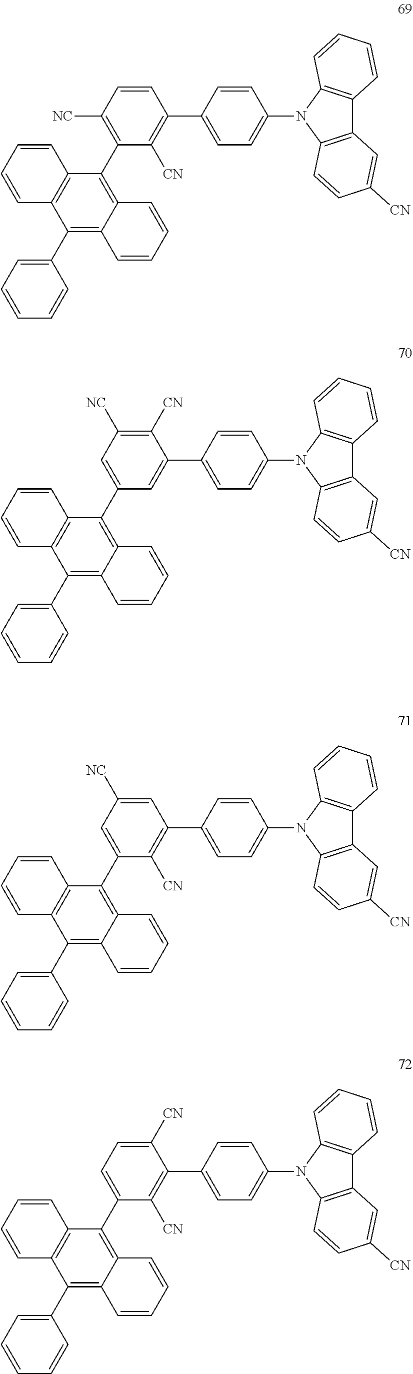 [SMILES: N#Cc1ccc2c(c1)c1ccccc1n2-c1ccc(-c2ccc(C#N)c(-c3c4ccccc4c(-c4ccccc4)c4ccccc34)c2C#N)cc1 ; N#Cc1ccc2c(c1)c1ccccc1n2-c1ccc(-c2cc(-c3c4ccccc4c(-c4ccccc4)c4ccccc34)cc(C#N)c2C#N)cc1 ; N#Cc1cc(-c2ccc(-n3c4ccccc4c4cc(C#N)ccc43)cc2)c(C#N)c(-c2c3ccccc3c(-c3ccccc3)c3ccccc23)c1 ; N#Cc1ccc2c(c1)c1ccccc1n2-c1ccc(-c2c(C#N)ccc(-c3c4ccccc4c(-c4ccccc4)c4ccccc34)c2C#N)cc1]

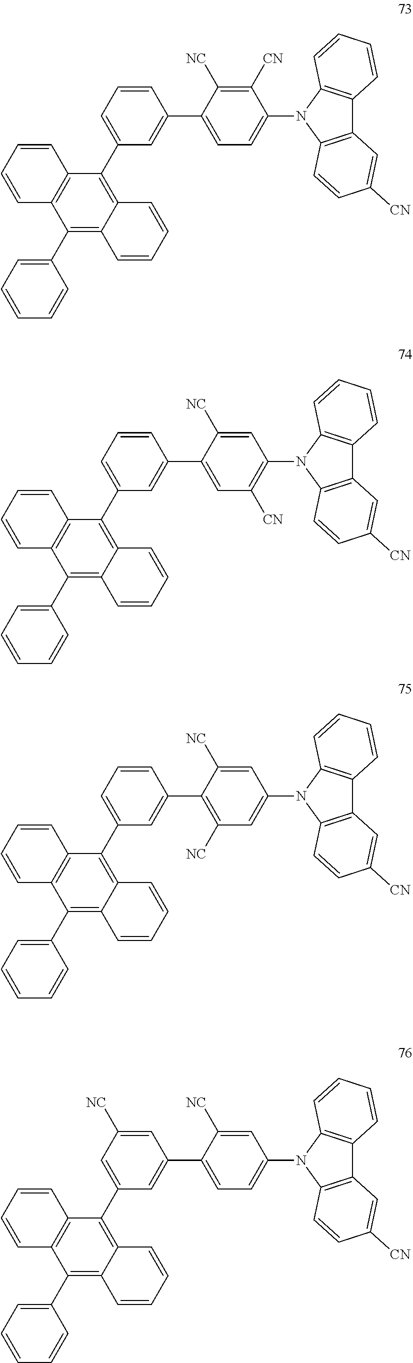 [SMILES: N#Cc1ccc2c(c1)c1ccccc1n2-c1ccc(-c2cccc(-c3c4ccccc4c(-c4ccccc4)c4ccccc34)c2)c(C#N)c1C#N ; N#Cc1ccc2c(c1)c1ccccc1n2-c1cc(C#N)c(-c2cccc(-c3c4ccccc4c(-c4ccccc4)c4ccccc34)c2)cc1C#N ; N#Cc1ccc2c(c1)c1ccccc1n2-c1cc(C#N)c(-c2cccc(-c3c4ccccc4c(-c4ccccc4)c4ccccc34)c2)c(C#N)c1 ; N#Cc1cc(-c2ccc(-n3c4ccccc4c4cc(C#N)ccc43)cc2C#N)cc(-c2c3ccccc3c(-c3ccccc3)c3ccccc23)c1]

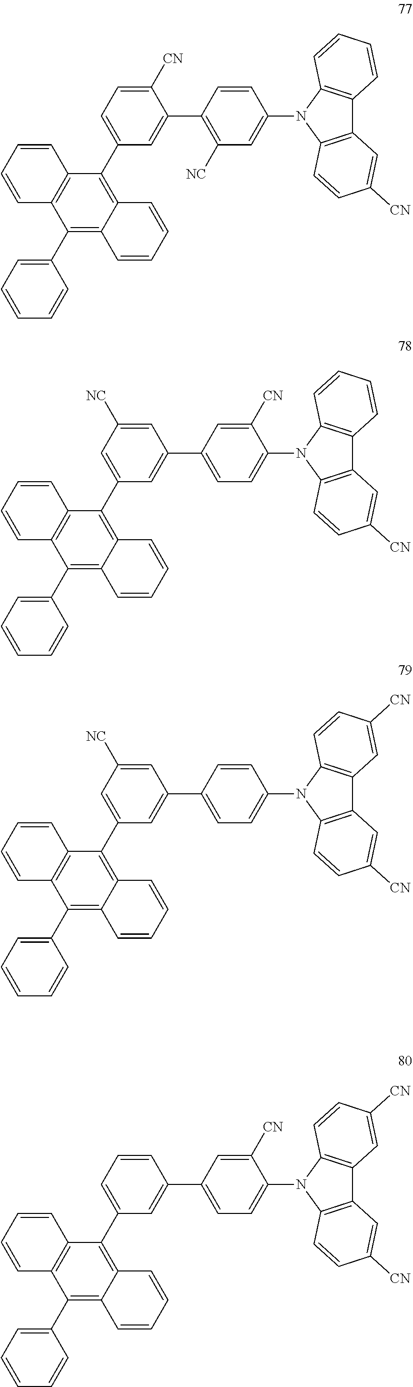 [SMILES: N#Cc1ccc2c(c1)c1ccccc1n2-c1ccc(-c2cc(-c3c4ccccc4c(-c4ccccc4)c4ccccc34)ccc2C#N)c(C#N)c1 ; N#Cc1cc(-c2ccc(-n3c4ccccc4c4cc(C#N)ccc43)c(C#N)c2)cc(-c2c3ccccc3c(-c3ccccc3)c3ccccc23)c1 ; N#Cc1cc(-c2ccc(-n3c4ccc(C#N)cc4c4cc(C#N)ccc43)cc2)cc(-c2c3ccccc3c(-c3ccccc3)c3ccccc23)c1 ; N#Cc1ccc2c(c1)c1cc(C#N)ccc1n2-c1ccc(-c2cccc(-c3c4ccccc4c(-c4ccccc4)c4ccccc34)c2)cc1C#N]

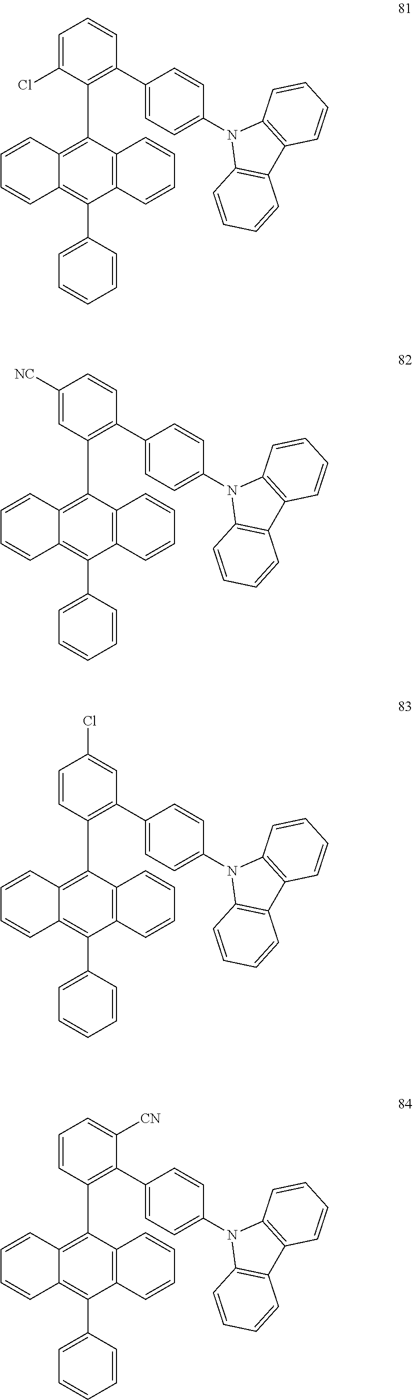 [SMILES: Clc1cccc(-c2ccc(-n3c4ccccc4c4ccccc43)cc2)c1-c1c2ccccc2c(-c2ccccc2)c2ccccc12 ; N#Cc1ccc(-c2ccc(-n3c4ccccc4c4ccccc43)cc2)c(-c2c3ccccc3c(-c3ccccc3)c3ccccc23)c1 ; Clc1ccc(-c2c3ccccc3c(-c3ccccc3)c3ccccc23)c(-c2ccc(-n3c4ccccc4c4ccccc43)cc2)c1 ; N#Cc1cccc(-c2c3ccccc3c(-c3ccccc3)c3ccccc23)c1-c1ccc(-n2c3ccccc3c3ccccc32)cc1]

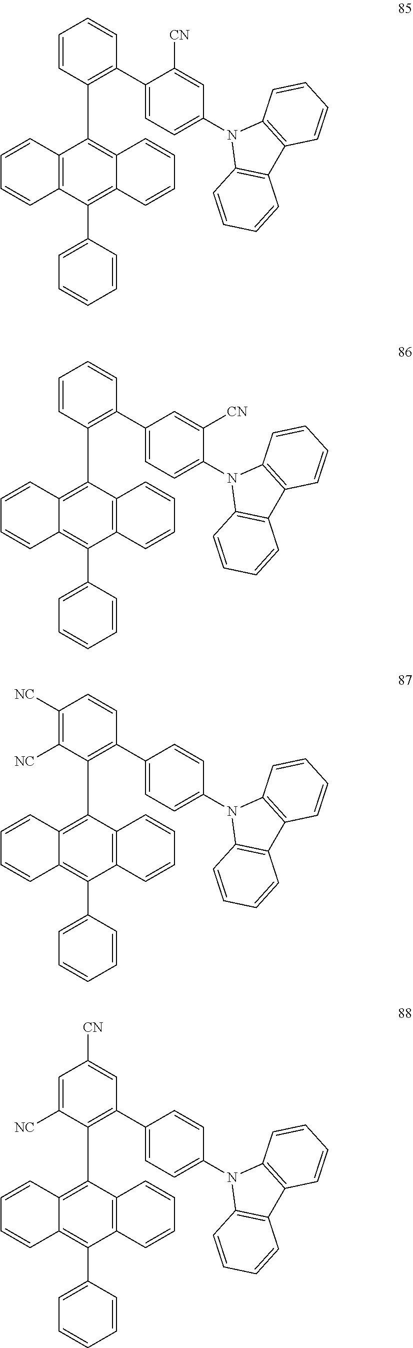

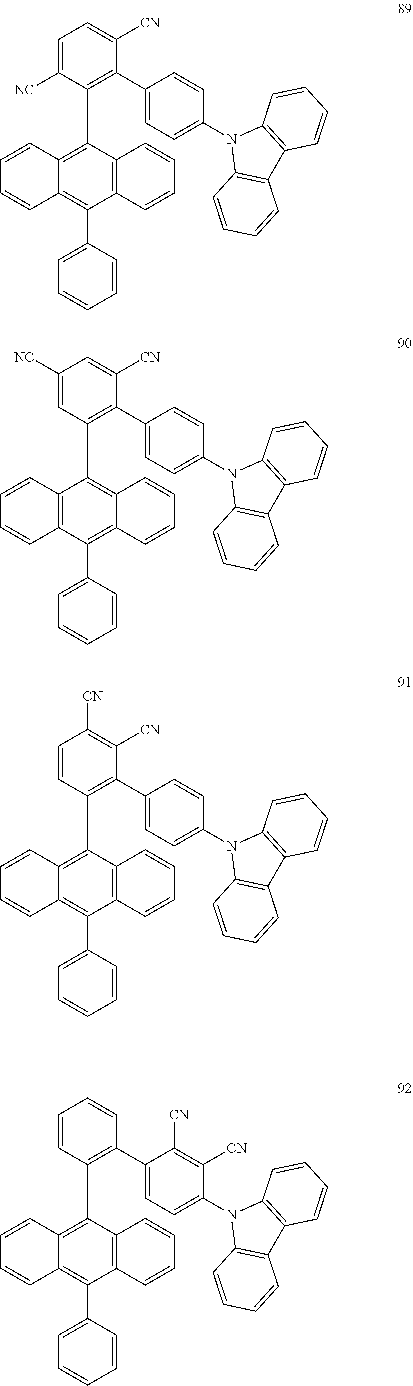 [SMILES: N#Cc1ccc(C#N)c(-c2c3ccccc3c(-c3ccccc3)c3ccccc23)c1-c1ccc(-n2c3ccccc3c3ccccc32)cc1 ; N#Cc1cc(C#N)c(-c2ccc(-n3c4ccccc4c4ccccc43)cc2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4ccccc43)cc2)c1C#N ; N#Cc1c(-c2ccccc2-c2c3ccccc3c(-c3ccccc3)c3ccccc23)ccc(-n2c3ccccc3c3ccccc32)c1C#N]

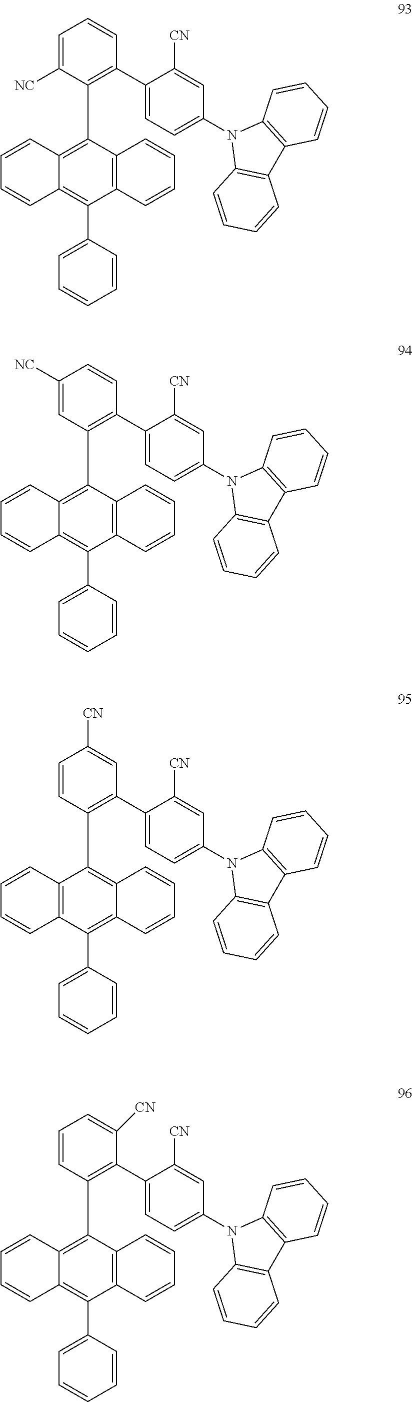 [SMILES: N#Cc1cc(-n2c3ccccc3c3ccccc32)ccc1-c1cccc(C#N)c1-c1c2ccccc2c(-c2ccccc2)c2ccccc12 ; N#Cc1ccc(-c2ccc(-n3c4ccccc4c4ccccc43)cc2C#N)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4ccccc43)cc2C#N)c1 ; N#Cc1cc(-n2c3ccccc3c3ccccc32)ccc1-c1c(C#N)cccc1-c1c2ccccc2c(-c2ccccc2)c2ccccc12]

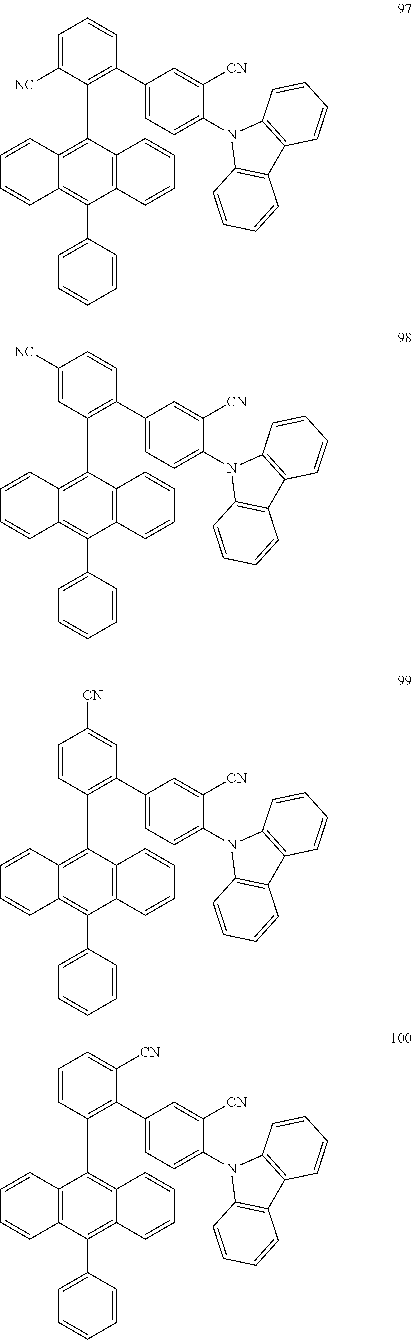

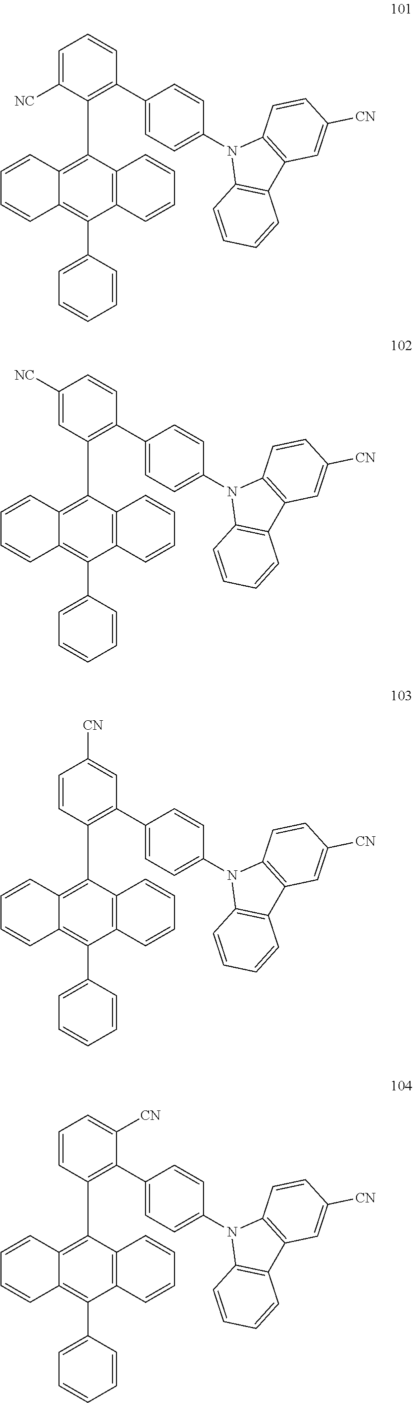 [SMILES: N#Cc1ccc2c(c1)c1ccccc1n2-c1ccc(-c2cccc(C#N)c2-c2c3ccccc3c(-c3ccccc3)c3ccccc23)cc1 ; N#Cc1ccc(-c2ccc(-n3c4ccccc4c4cc(C#N)ccc43)cc2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4cc(C#N)ccc43)cc2)c1 ; N#Cc1ccc2c(c1)c1ccccc1n2-c1ccc(-c2c(C#N)cccc2-c2c3ccccc3c(-c3ccccc3)c3ccccc23)cc1]

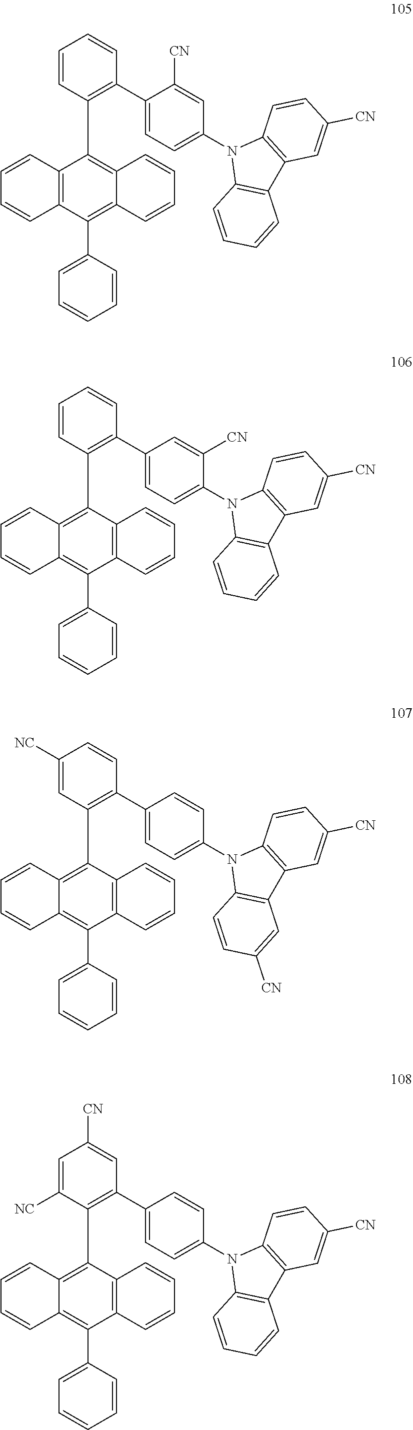 [SMILES: N#Cc1ccc2c(c1)c1ccccc1n2-c1ccc(-c2ccccc2-c2c3ccccc3c(-c3ccccc3)c3ccccc23)c(C#N)c1 ; N#Cc1ccc2c(c1)c1ccccc1n2-c1ccc(-c2ccccc2-c2c3ccccc3c(-c3ccccc3)c3ccccc23)cc1C#N ; N#Cc1ccc(-c2ccc(-n3c4ccc(C#N)cc4c4cc(C#N)ccc43)cc2)c(-c2c3ccccc3c(-c3ccccc3)c3ccccc23)c1 ; N#Cc1cc(C#N)c(-c2c3ccccc3c(-c3ccccc3)c3ccccc23)c(-c2ccc(-n3c4ccccc4c4cc(C#N)ccc43)cc2)c1]

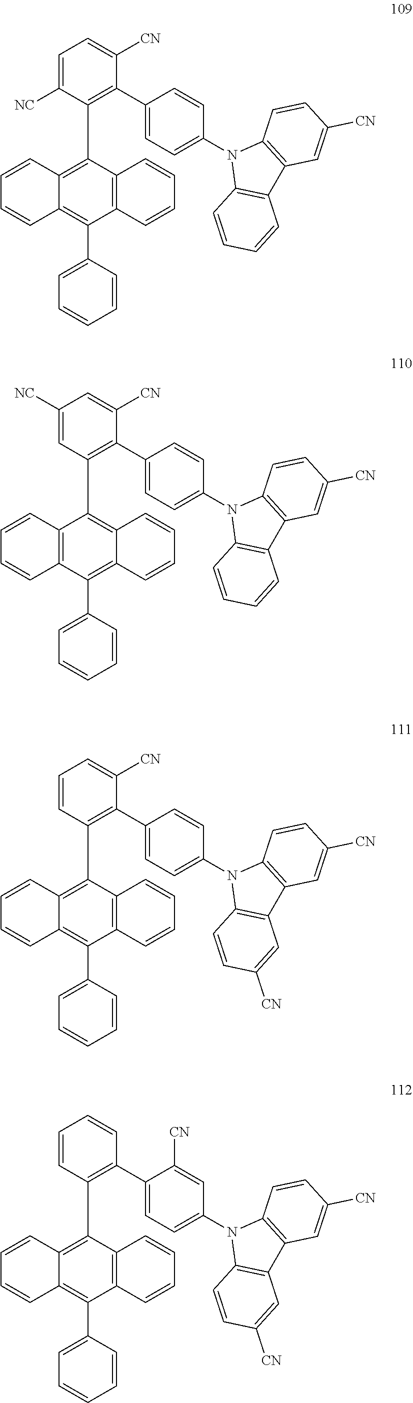 [SMILES: N#Cc1ccc2c(c1)c1ccccc1n2-c1ccc(-c2c(C#N)ccc(C#N)c2-c2c3ccccc3c(-c3ccccc3)c3ccccc23)cc1 ; N#Cc1cc(C#N)c(-c2ccc(-n3c4ccccc4c4cc(C#N)ccc43)cc2)c(-c2c3ccccc3c(-c3ccccc3)c3ccccc23)c1 ; N#Cc1ccc2c(c1)c1cc(C#N)ccc1n2-c1ccc(-c2c(C#N)cccc2-c2c3ccccc3c(-c3ccccc3)c3ccccc23)cc1 ; N#Cc1ccc2c(c1)c1cc(C#N)ccc1n2-c1ccc(-c2ccccc2-c2c3ccccc3c(-c3ccccc3)c3ccccc23)c(C#N)c1]

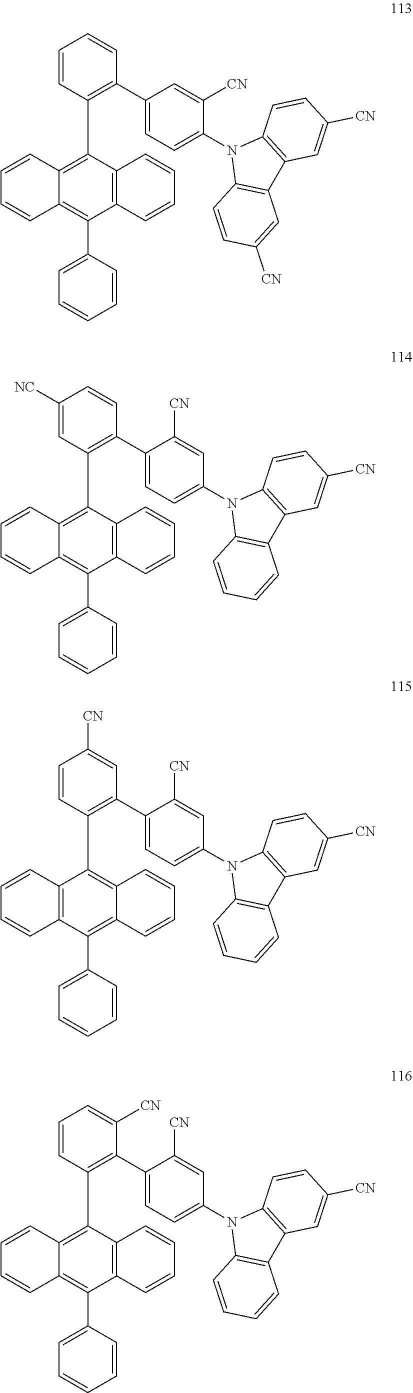

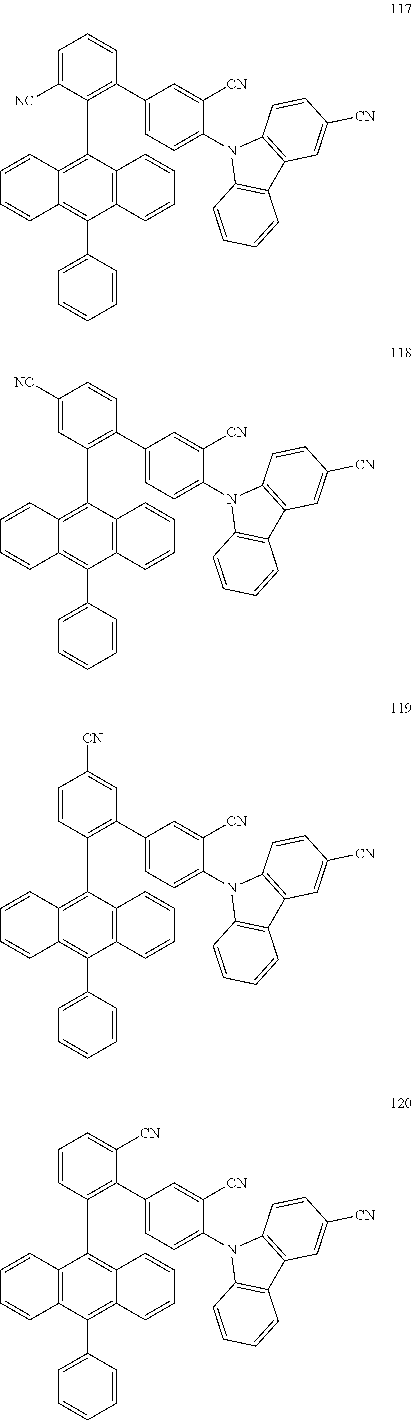 [SMILES: N#Cc1ccc2c(c1)c1ccccc1n2-c1ccc(-c2cccc(C#N)c2-c2c3ccccc3c(-c3ccccc3)c3ccccc23)cc1C#N ; N#Cc1ccc(-c2ccc(-n3c4ccccc4c4cc(C#N)ccc43)c(C#N)c2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4cc(C#N)ccc43)c(C#N)c2)c1 ; N#Cc1ccc2c(c1)c1ccccc1n2-c1ccc(-c2c(C#N)cccc2-c2c3ccccc3c(-c3ccccc3)c3ccccc23)cc1C#N]

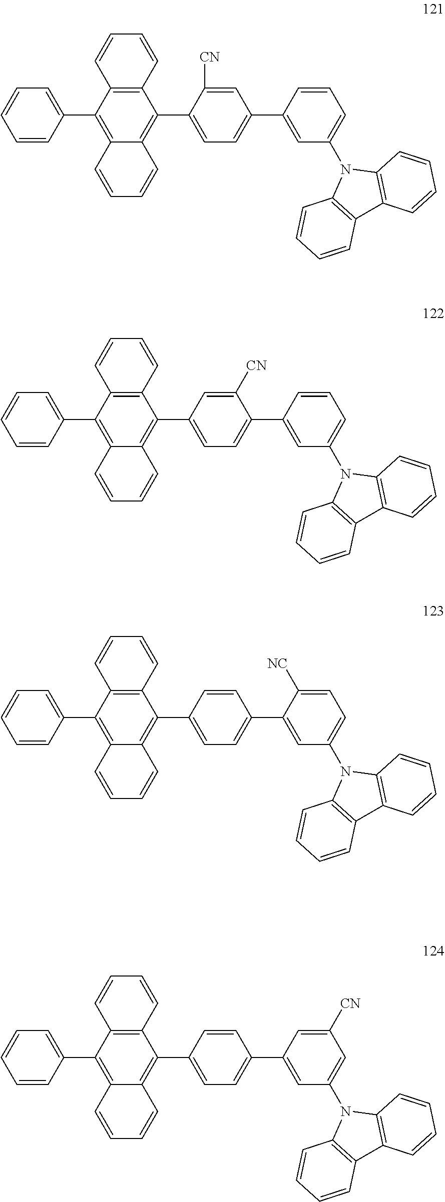 [SMILES: N#Cc1cc(-c2cccc(-n3c4ccccc4c4ccccc43)c2)ccc1-c1c2ccccc2c(-c2ccccc2)c2ccccc12 ; N#Cc1cc(-c2c3ccccc3c(-c3ccccc3)c3ccccc23)ccc1-c1cccc(-n2c3ccccc3c3ccccc32)c1 ; N#Cc1ccc(-n2c3ccccc3c3ccccc32)cc1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)cc(-n2c3ccccc3c3ccccc32)c1]

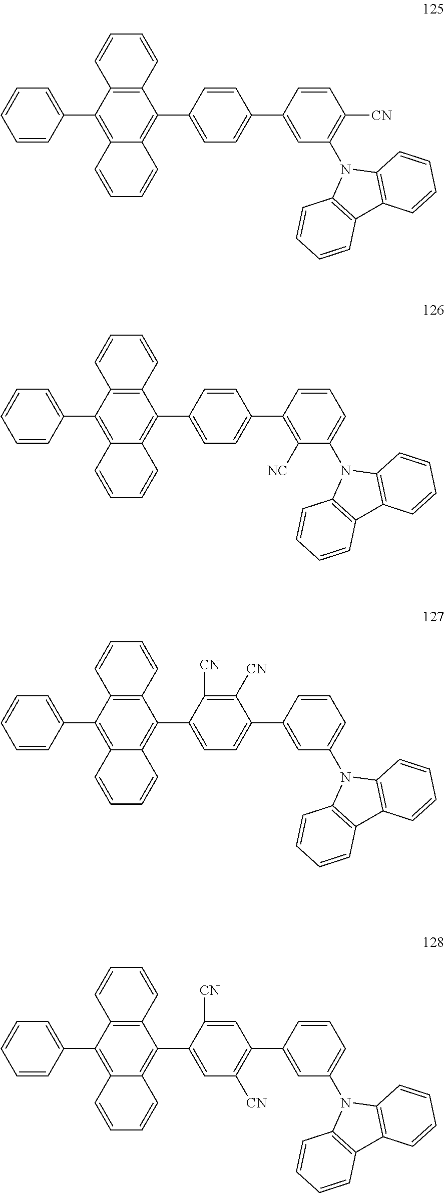 [SMILES: N#Cc1ccc(-c2ccc(-c3c4ccccc4c(-c4ccccc4)c4ccccc34)cc2)cc1-n1c2ccccc2c2ccccc21 ; N#Cc1c(-c2ccc(-c3c4ccccc4c(-c4ccccc4)c4ccccc34)cc2)cccc1-n1c2ccccc2c2ccccc21 ; N#Cc1c(-c2cccc(-n3c4ccccc4c4ccccc43)c2)ccc(-c2c3ccccc3c(-c3ccccc3)c3ccccc23)c1C#N ; N#Cc1cc(-c2c3ccccc3c(-c3ccccc3)c3ccccc23)c(C#N)cc1-c1cccc(-n2c3ccccc3c3ccccc32)c1]

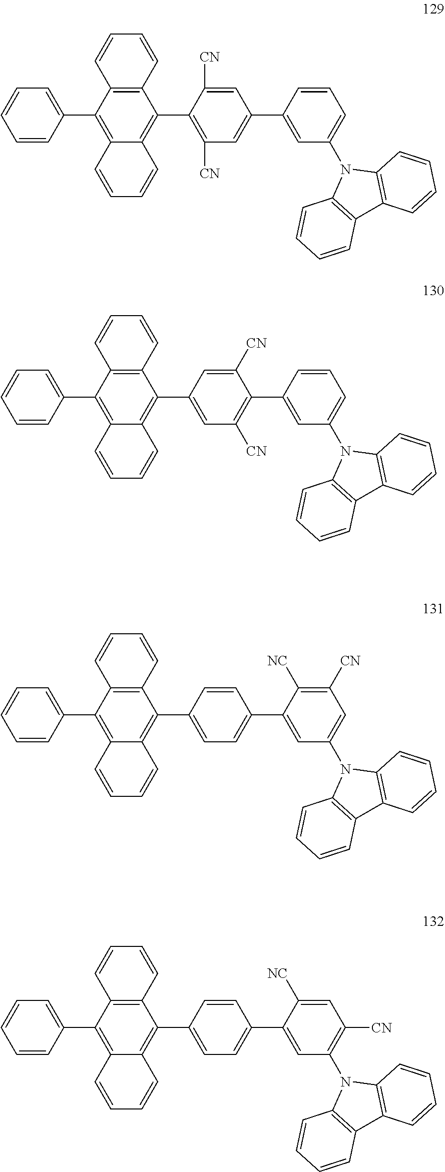 [SMILES: N#Cc1cc(-c2cccc(-n3c4ccccc4c4ccccc43)c2)cc(C#N)c1-c1c2ccccc2c(-c2ccccc2)c2ccccc12 ; N#Cc1cc(-c2c3ccccc3c(-c3ccccc3)c3ccccc23)cc(C#N)c1-c1cccc(-n2c3ccccc3c3ccccc32)c1 ; N#Cc1cc(-n2c3ccccc3c3ccccc32)cc(-c2ccc(-c3c4ccccc4c(-c4ccccc4)c4ccccc34)cc2)c1C#N ; N#Cc1cc(C#N)c(-n2c3ccccc3c3ccccc32)cc1-c1ccc(-c2c3ccccc3c(-c3ccccc3)c3ccccc23)cc1]

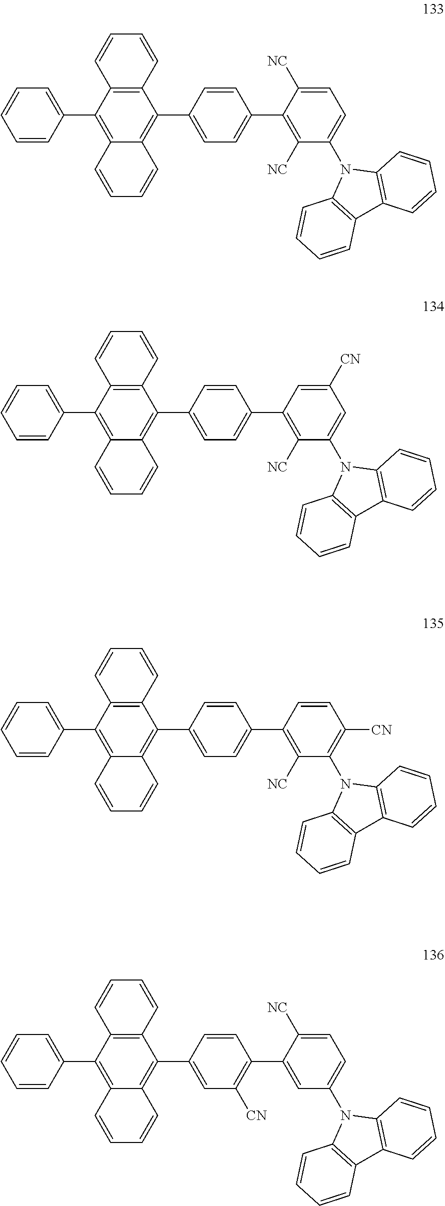 [SMILES: N#Cc1ccc(-n2c3ccccc3c3ccccc32)c(C#N)c1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)c(C#N)c(-n2c3ccccc3c3ccccc32)c1 ; N#Cc1ccc(-c2ccc(-c3c4ccccc4c(-c4ccccc4)c4ccccc34)cc2)c(C#N)c1-n1c2ccccc2c2ccccc21 ; N#Cc1cc(-c2c3ccccc3c(-c3ccccc3)c3ccccc23)ccc1-c1cc(-n2c3ccccc3c3ccccc32)ccc1C#N]

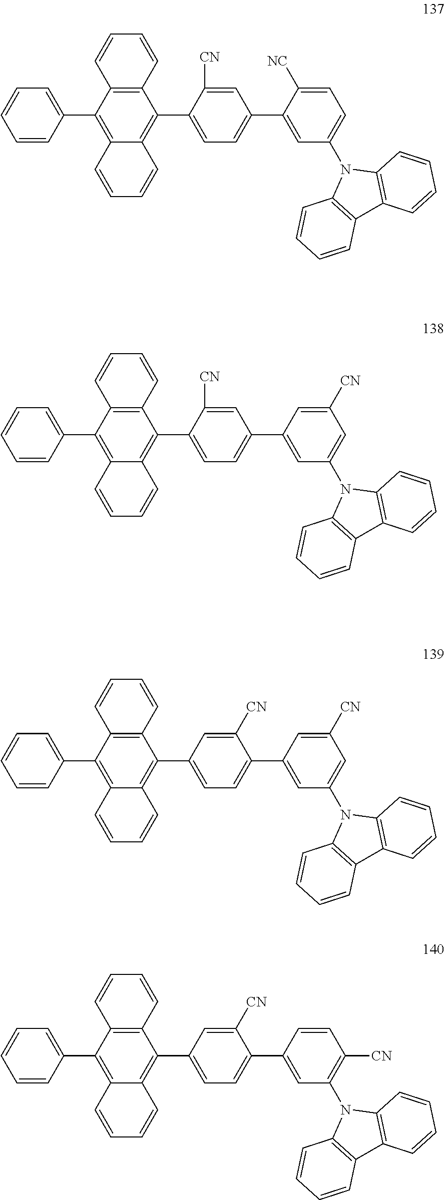 [SMILES: N#Cc1ccc(-n2c3ccccc3c3ccccc32)cc1-c1ccc(-c2c3ccccc3c(-c3ccccc3)c3ccccc23)c(C#N)c1 ; N#Cc1cc(-c2ccc(-c3c4ccccc4c(-c4ccccc4)c4ccccc34)c(C#N)c2)cc(-n2c3ccccc3c3ccccc32)c1 ; N#Cc1cc(-c2ccc(-c3c4ccccc4c(-c4ccccc4)c4ccccc34)cc2C#N)cc(-n2c3ccccc3c3ccccc32)c1 ; N#Cc1cc(-c2c3ccccc3c(-c3ccccc3)c3ccccc23)ccc1-c1ccc(C#N)c(-n2c3ccccc3c3ccccc32)c1]

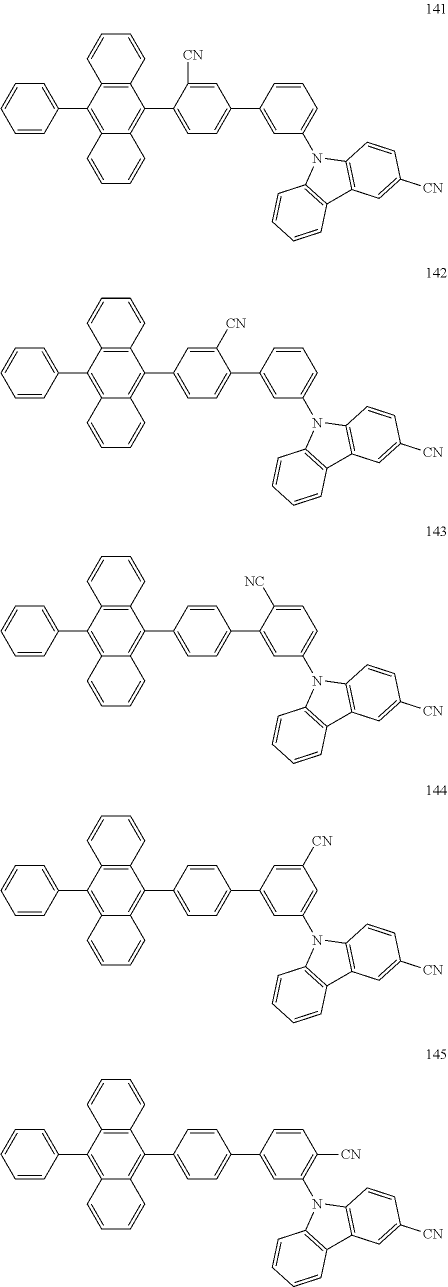 [SMILES: N#Cc1ccc2c(c1)c1ccccc1n2-c1cccc(-c2ccc(-c3c4ccccc4c(-c4ccccc4)c4ccccc34)c(C#N)c2)c1 ; N#Cc1ccc2c(c1)c1ccccc1n2-c1cccc(-c2ccc(-c3c4ccccc4c(-c4ccccc4)c4ccccc34)cc2C#N)c1 ; N#Cc1ccc2c(c1)c1ccccc1n2-c1ccc(C#N)c(-c2ccc(-c3c4ccccc4c(-c4ccccc4)c4ccccc34)cc2)c1 ; N#Cc1cc(-c2ccc(-c3c4ccccc4c(-c4ccccc4)c4ccccc34)cc2)cc(-n2c3ccccc3c3cc(C#N)ccc32)c1 ; N#Cc1ccc2c(c1)c1ccccc1n2-c1cc(-c2ccc(-c3c4ccccc4c(-c4ccccc4)c4ccccc34)cc2)ccc1C#N]

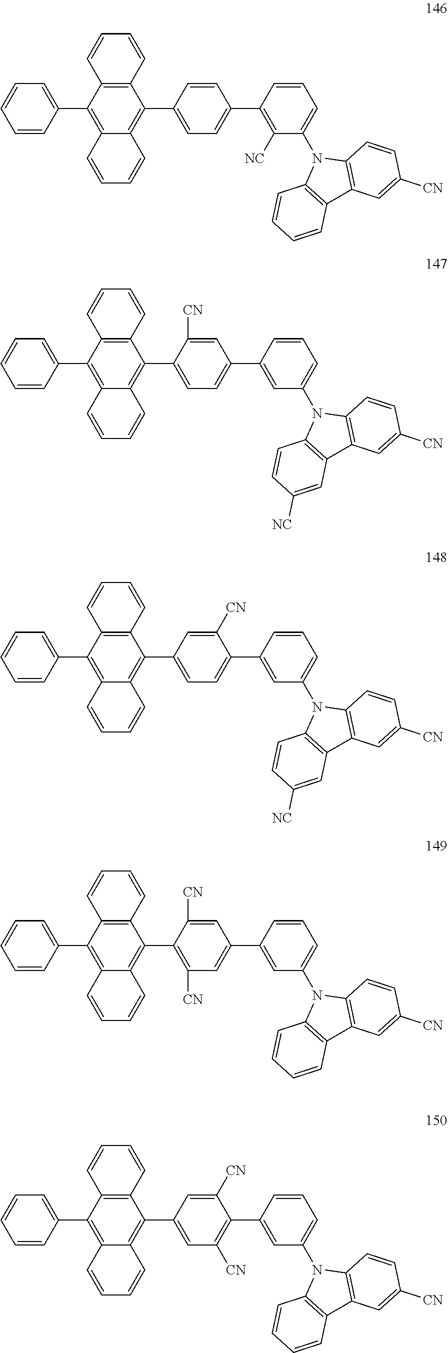 [SMILES: N#Cc1ccc2c(c1)c1ccccc1n2-c1cccc(-c2ccc(-c3c4ccccc4c(-c4ccccc4)c4ccccc34)cc2)c1C#N ; N#Cc1ccc2c(c1)c1cc(C#N)ccc1n2-c1cccc(-c2ccc(-c3c4ccccc4c(-c4ccccc4)c4ccccc34)c(C#N)c2)c1 ; N#Cc1ccc2c(c1)c1cc(C#N)ccc1n2-c1cccc(-c2ccc(-c3c4ccccc4c(-c4ccccc4)c4ccccc34)cc2C#N)c1 ; N#Cc1ccc2c(c1)c1ccccc1n2-c1cccc(-c2cc(C#N)c(-c3c4ccccc4c(-c4ccccc4)c4ccccc34)c(C#N)c2)c1 ; N#Cc1ccc2c(c1)c1ccccc1n2-c1cccc(-c2c(C#N)cc(-c3c4ccccc4c(-c4ccccc4)c4ccccc34)cc2C#N)c1]

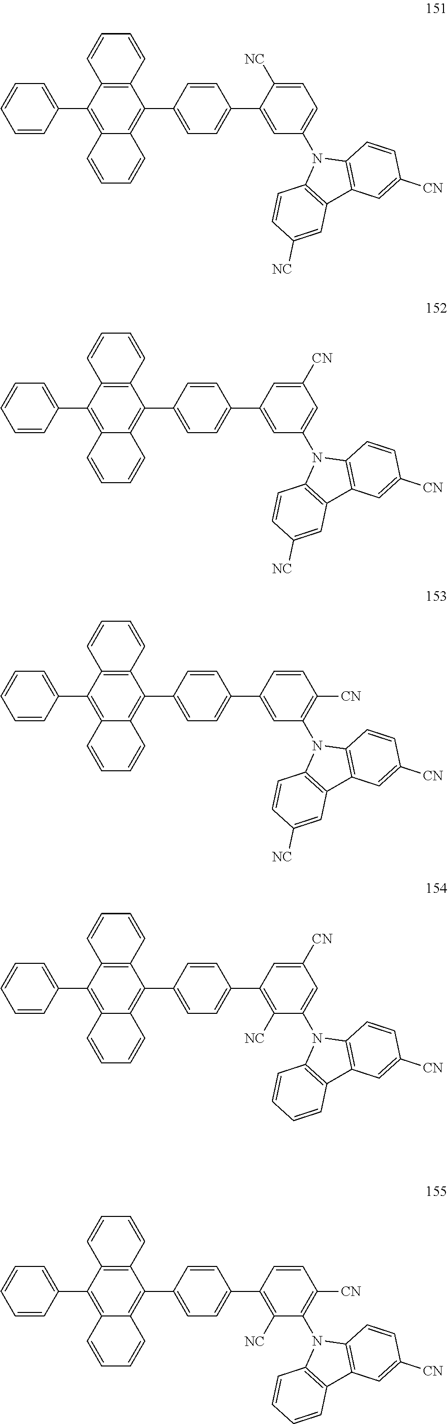 [SMILES: N#Cc1ccc2c(c1)c1cc(C#N)ccc1n2-c1ccc(C#N)c(-c2ccc(-c3c4ccccc4c(-c4ccccc4)c4ccccc34)cc2)c1 ; N#Cc1cc(-c2ccc(-c3c4ccccc4c(-c4ccccc4)c4ccccc34)cc2)cc(-n2c3ccc(C#N)cc3c3cc(C#N)ccc32)c1 ; N#Cc1ccc2c(c1)c1cc(C#N)ccc1n2-c1cc(-c2ccc(-c3c4ccccc4c(-c4ccccc4)c4ccccc34)cc2)ccc1C#N ; N#Cc1cc(-c2ccc(-c3c4ccccc4c(-c4ccccc4)c4ccccc34)cc2)c(C#N)c(-n2c3ccccc3c3cc(C#N)ccc32)c1 ; N#Cc1ccc2c(c1)c1ccccc1n2-c1c(C#N)ccc(-c2ccc(-c3c4ccccc4c(-c4ccccc4)c4ccccc34)cc2)c1C#N]

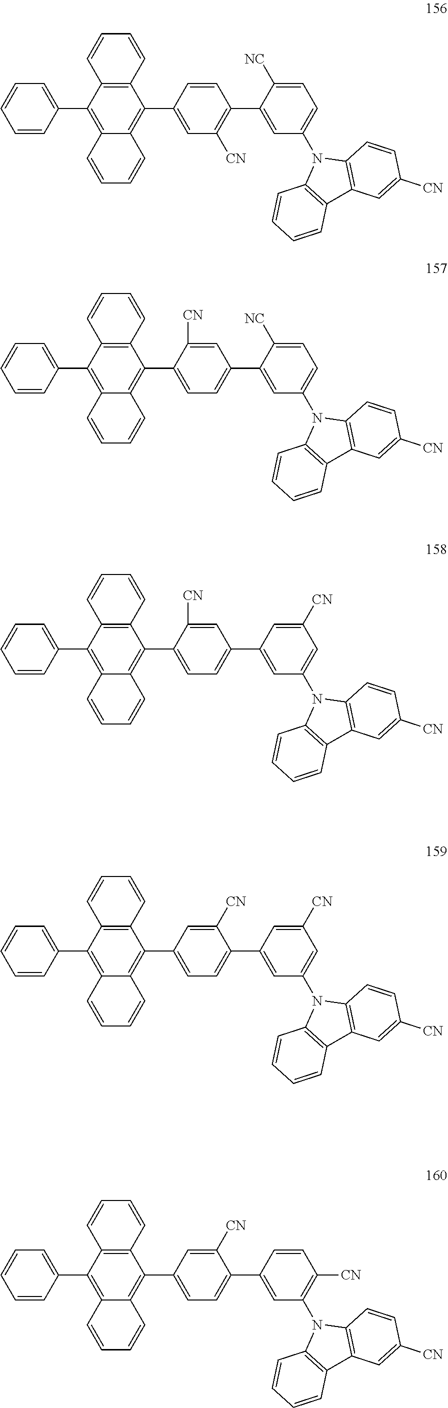 [SMILES: N#Cc1ccc2c(c1)c1ccccc1n2-c1ccc(C#N)c(-c2ccc(-c3c4ccccc4c(-c4ccccc4)c4ccccc34)cc2C#N)c1 ; N#Cc1ccc2c(c1)c1ccccc1n2-c1ccc(C#N)c(-c2ccc(-c3c4ccccc4c(-c4ccccc4)c4ccccc34)c(C#N)c2)c1 ; N#Cc1cc(-c2ccc(-c3c4ccccc4c(-c4ccccc4)c4ccccc34)c(C#N)c2)cc(-n2c3ccccc3c3cc(C#N)ccc32)c1 ; N#Cc1cc(-c2ccc(-c3c4ccccc4c(-c4ccccc4)c4ccccc34)cc2C#N)cc(-n2c3ccccc3c3cc(C#N)ccc32)c1 ; N#Cc1ccc2c(c1)c1ccccc1n2-c1cc(-c2ccc(-c3c4ccccc4c(-c4ccccc4)c4ccccc34)cc2C#N)ccc1C#N]

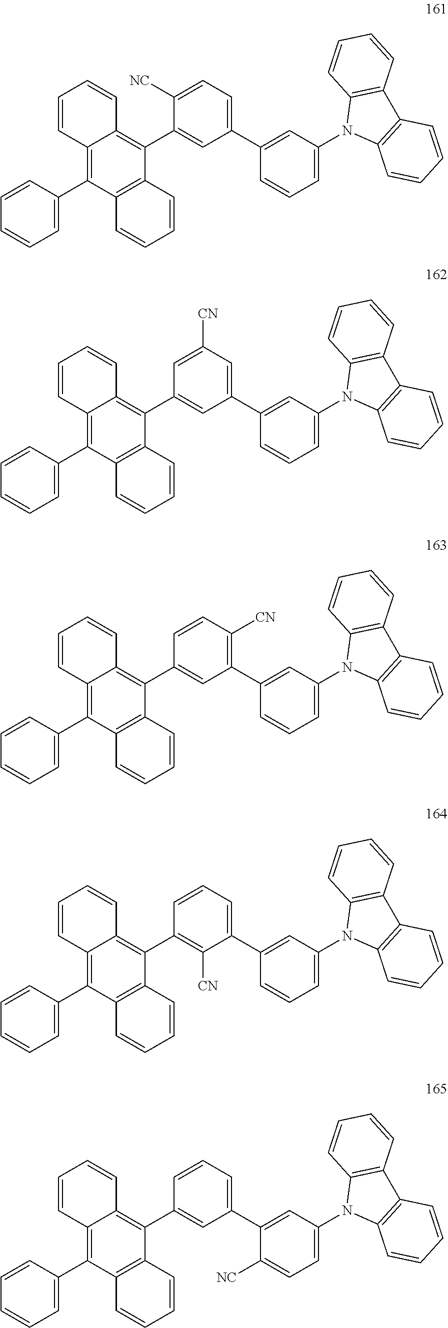 [SMILES: N#Cc1ccc(-c2cccc(-n3c4ccccc4c4ccccc43)c2)cc1-c1c2ccccc2c(-c2ccccc2)c2ccccc12 ; N#Cc1cc(-c2cccc(-n3c4ccccc4c4ccccc43)c2)cc(-c2c3ccccc3c(-c3ccccc3)c3ccccc23)c1 ; N#Cc1ccc(-c2c3ccccc3c(-c3ccccc3)c3ccccc23)cc1-c1cccc(-n2c3ccccc3c3ccccc32)c1 ; N#Cc1c(-c2cccc(-n3c4ccccc4c4ccccc43)c2)cccc1-c1c2ccccc2c(-c2ccccc2)c2ccccc12 ; N#Cc1ccc(-n2c3ccccc3c3ccccc32)cc1-c1cccc(-c2c3ccccc3c(-c3ccccc3)c3ccccc23)c1]

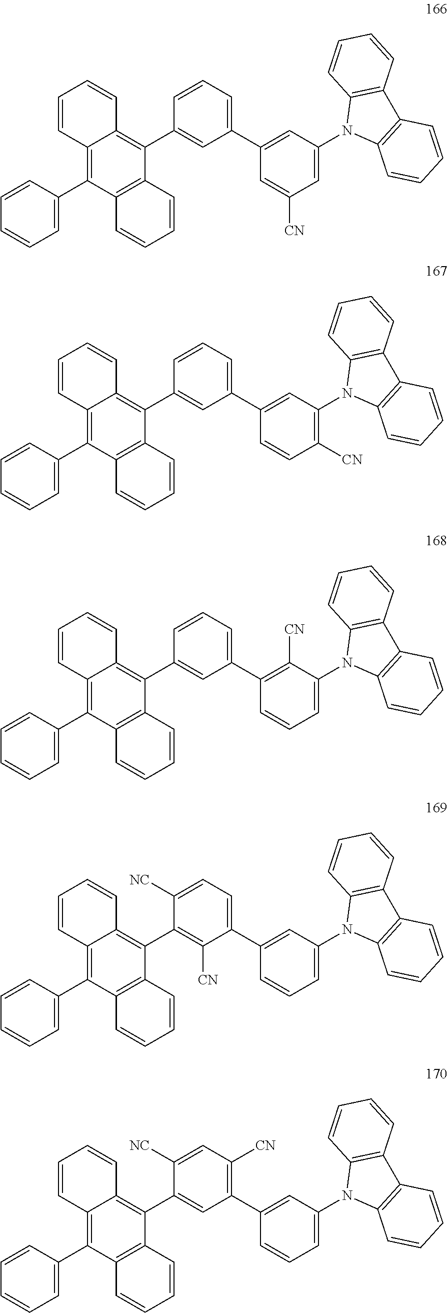 [SMILES: N#Cc1cc(-c2cccc(-c3c4ccccc4c(-c4ccccc4)c4ccccc34)c2)cc(-n2c3ccccc3c3ccccc32)c1 ; N#Cc1ccc(-c2cccc(-c3c4ccccc4c(-c4ccccc4)c4ccccc34)c2)cc1-n1c2ccccc2c2ccccc21 ; N#Cc1c(-c2cccc(-c3c4ccccc4c(-c4ccccc4)c4ccccc34)c2)cccc1-n1c2ccccc2c2ccccc21 ; N#Cc1ccc(-c2cccc(-n3c4ccccc4c4ccccc43)c2)c(C#N)c1-c1c2ccccc2c(-c2ccccc2)c2ccccc12 ; N#Cc1cc(C#N)c(-c2c3ccccc3c(-c3ccccc3)c3ccccc23)cc1-c1cccc(-n2c3ccccc3c3ccccc32)c1]

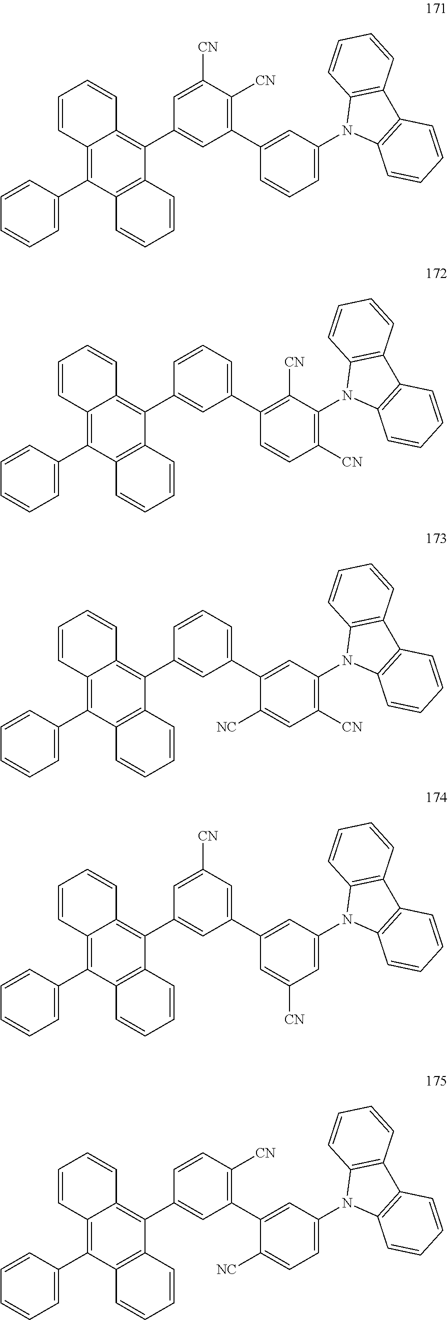 [SMILES: N#Cc1cc(-c2c3ccccc3c(-c3ccccc3)c3ccccc23)cc(-c2cccc(-n3c4ccccc4c4ccccc43)c2)c1C#N ; N#Cc1ccc(-c2cccc(-c3c4ccccc4c(-c4ccccc4)c4ccccc34)c2)c(C#N)c1-n1c2ccccc2c2ccccc21 ; N#Cc1cc(C#N)c(-n2c3ccccc3c3ccccc32)cc1-c1cccc(-c2c3ccccc3c(-c3ccccc3)c3ccccc23)c1 ; N#Cc1cc(-c2cc(C#N)cc(-n3c4ccccc4c4ccccc43)c2)cc(-c2c3ccccc3c(-c3ccccc3)c3ccccc23)c1 ; N#Cc1ccc(-c2c3ccccc3c(-c3ccccc3)c3ccccc23)cc1-c1cc(-n2c3ccccc3c3ccccc32)ccc1C#N]

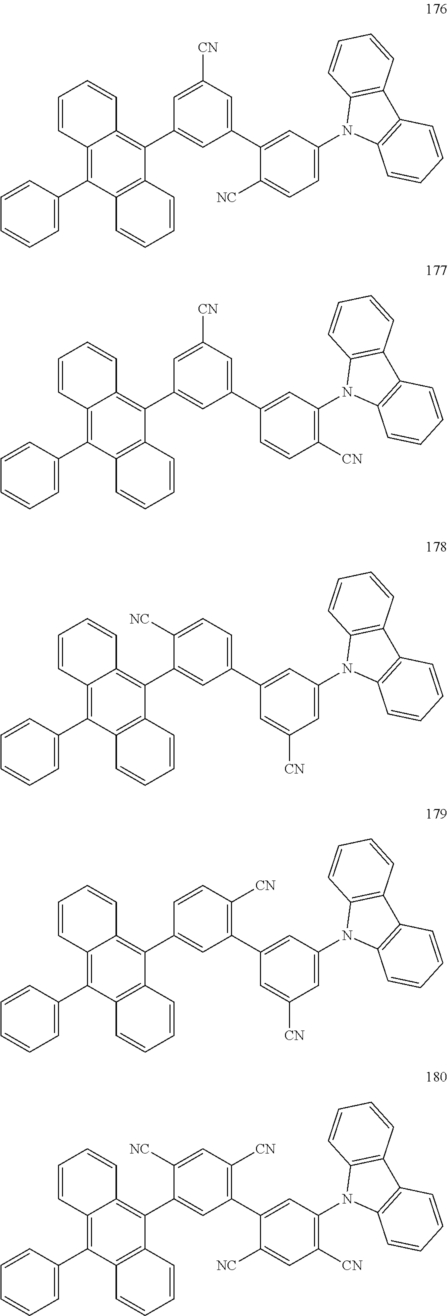 [SMILES: N#Cc1cc(-c2cc(-n3c4ccccc4c4ccccc43)ccc2C#N)cc(-c2c3ccccc3c(-c3ccccc3)c3ccccc23)c1 ; N#Cc1cc(-c2ccc(C#N)c(-n3c4ccccc4c4ccccc43)c2)cc(-c2c3ccccc3c(-c3ccccc3)c3ccccc23)c1 ; N#Cc1cc(-c2ccc(C#N)c(-c3c4ccccc4c(-c4ccccc4)c4ccccc34)c2)cc(-n2c3ccccc3c3ccccc32)c1 ; N#Cc1cc(-c2cc(-c3c4ccccc4c(-c4ccccc4)c4ccccc34)ccc2C#N)cc(-n2c3ccccc3c3ccccc32)c1 ; N#Cc1cc(C#N)c(-c2c3ccccc3c(-c3ccccc3)c3ccccc23)cc1-c1cc(-n2c3ccccc3c3ccccc32)c(C#N)cc1C#N]

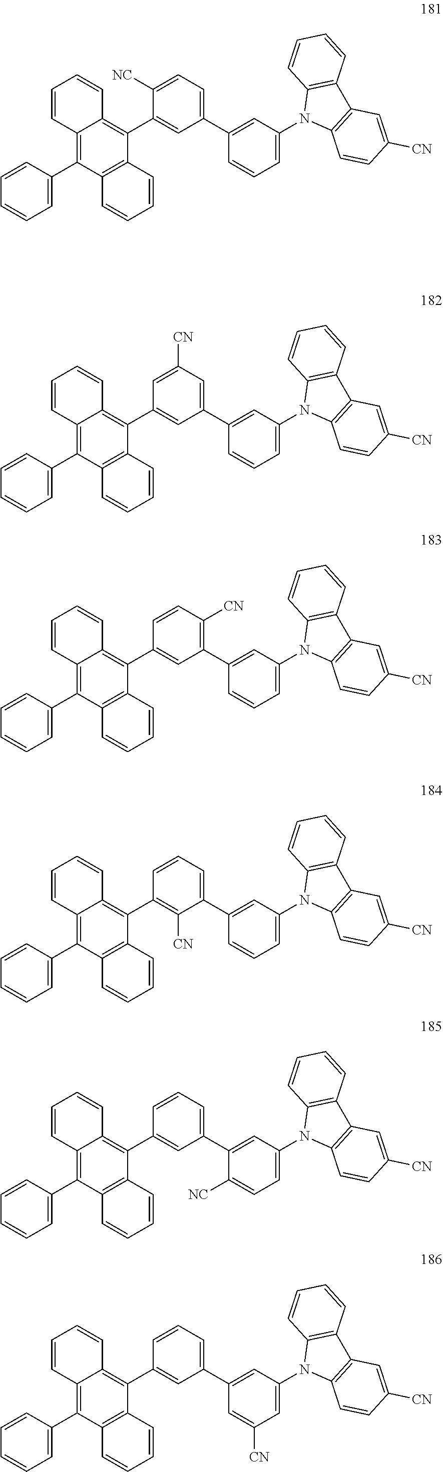

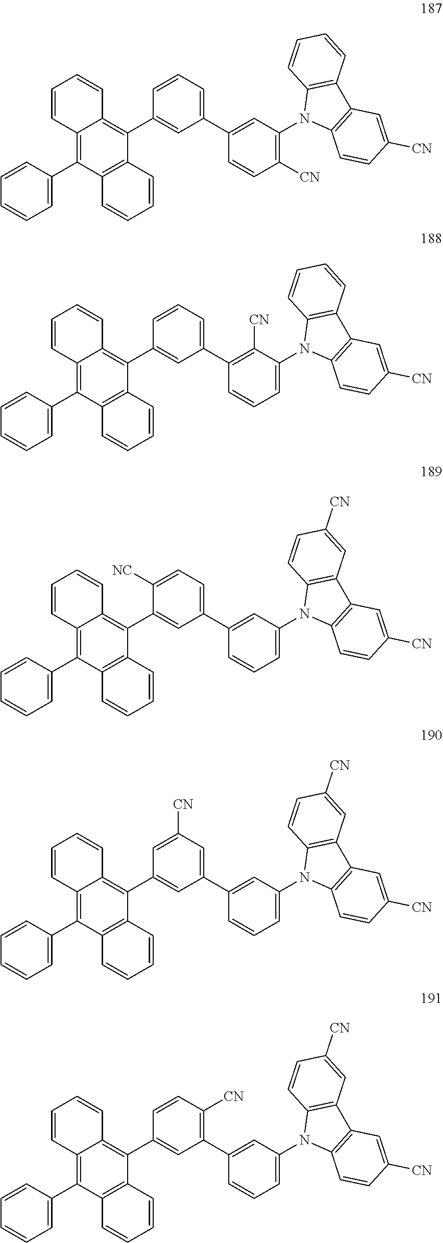 [SMILES: N#Cc1ccc2c(c1)c1ccccc1n2-c1cc(-c2cccc(-c3c4ccccc4c(-c4ccccc4)c4ccccc34)c2)ccc1C#N ; N#Cc1ccc2c(c1)c1ccccc1n2-c1cccc(-c2cccc(-c3c4ccccc4c(-c4ccccc4)c4ccccc34)c2)c1C#N ; N#Cc1ccc2c(c1)c1cc(C#N)ccc1n2-c1cccc(-c2ccc(C#N)c(-c3c4ccccc4c(-c4ccccc4)c4ccccc34)c2)c1 ; N#Cc1cc(-c2cccc(-n3c4ccc(C#N)cc4c4cc(C#N)ccc43)c2)cc(-c2c3ccccc3c(-c3ccccc3)c3ccccc23)c1 ; N#Cc1ccc2c(c1)c1cc(C#N)ccc1n2-c1cccc(-c2cc(-c3c4ccccc4c(-c4ccccc4)c4ccccc34)ccc2C#N)c1]

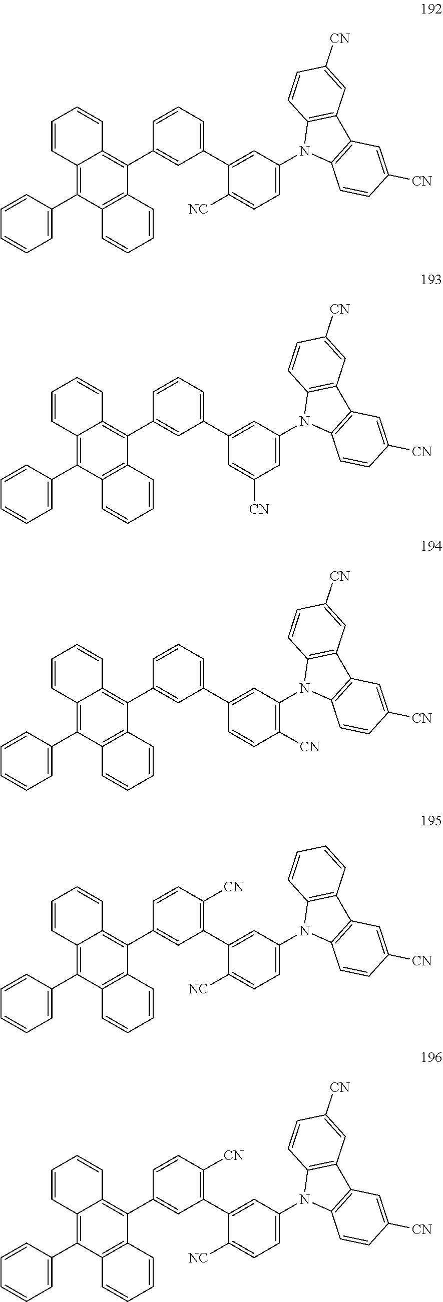 [SMILES: N#Cc1ccc2c(c1)c1cc(C#N)ccc1n2-c1ccc(C#N)c(-c2cccc(-c3c4ccccc4c(-c4ccccc4)c4ccccc34)c2)c1 ; N#Cc1cc(-c2cccc(-c3c4ccccc4c(-c4ccccc4)c4ccccc34)c2)cc(-n2c3ccc(C#N)cc3c3cc(C#N)ccc32)c1 ; N#Cc1ccc2c(c1)c1cc(C#N)ccc1n2-c1cc(-c2cccc(-c3c4ccccc4c(-c4ccccc4)c4ccccc34)c2)ccc1C#N ; N#Cc1ccc2c(c1)c1ccccc1n2-c1ccc(C#N)c(-c2cc(-c3c4ccccc4c(-c4ccccc4)c4ccccc34)ccc2C#N)c1 ; N#Cc1ccc2c(c1)c1cc(C#N)ccc1n2-c1ccc(C#N)c(-c2cc(-c3c4ccccc4c(-c4ccccc4)c4ccccc34)ccc2C#N)c1]

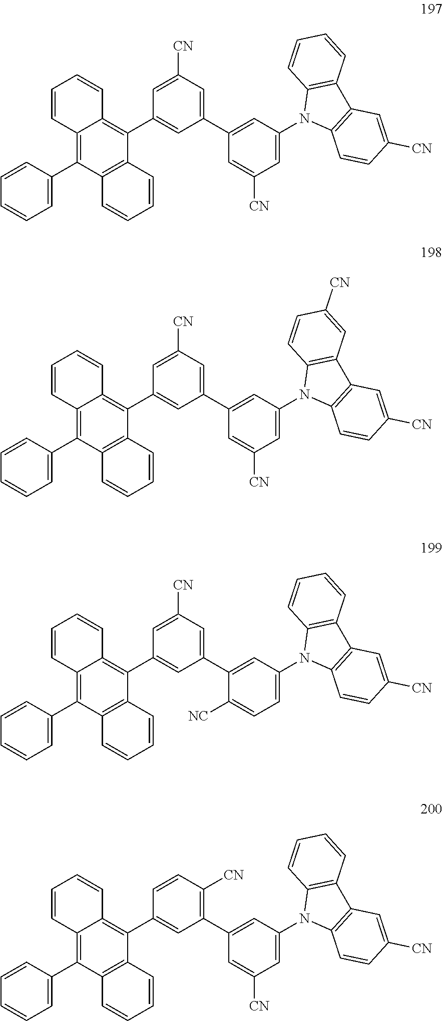 [SMILES: N#Cc1cc(-c2cc(C#N)cc(-n3c4ccccc4c4cc(C#N)ccc43)c2)cc(-c2c3ccccc3c(-c3ccccc3)c3ccccc23)c1 ; N#Cc1cc(-c2cc(C#N)cc(-n3c4ccc(C#N)cc4c4cc(C#N)ccc43)c2)cc(-c2c3ccccc3c(-c3ccccc3)c3ccccc23)c1 ; N#Cc1cc(-c2cc(-n3c4ccccc4c4cc(C#N)ccc43)ccc2C#N)cc(-c2c3ccccc3c(-c3ccccc3)c3ccccc23)c1 ; N#Cc1cc(-c2cc(-c3c4ccccc4c(-c4ccccc4)c4ccccc34)ccc2C#N)cc(-n2c3ccccc3c3cc(C#N)ccc32)c1]

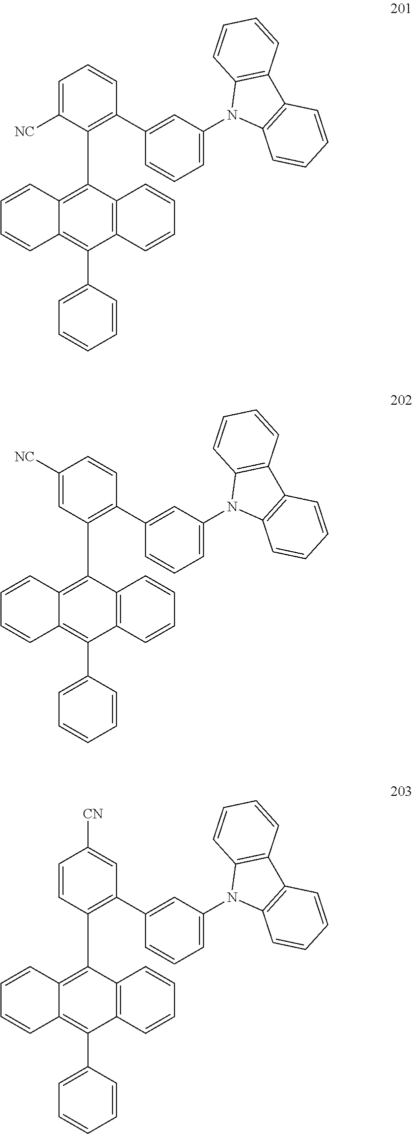 [SMILES: N#Cc1cccc(-c2cccc(-n3c4ccccc4c4ccccc43)c2)c1-c1c2ccccc2c(-c2ccccc2)c2ccccc12 ; N#Cc1ccc(-c2cccc(-n3c4ccccc4c4ccccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccccc4c4ccccc43)c2)c1]

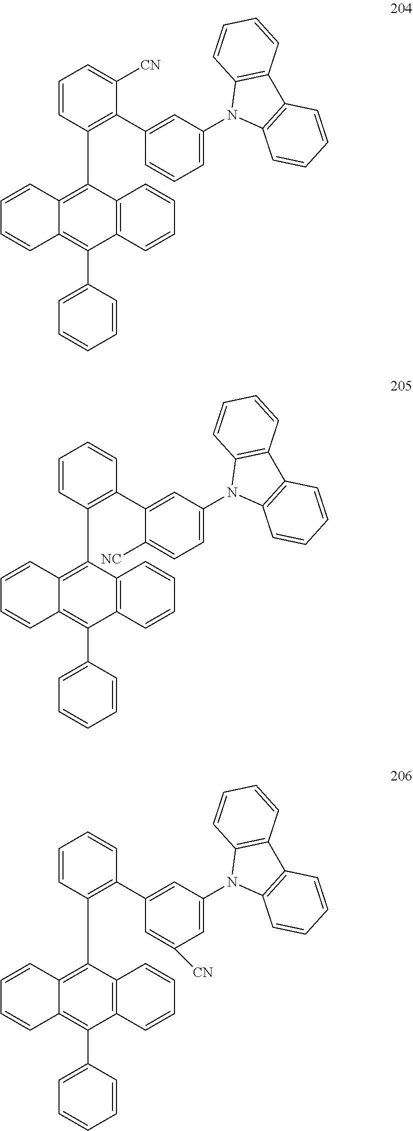 [SMILES: N#Cc1cccc(-c2c3ccccc3c(-c3ccccc3)c3ccccc23)c1-c1cccc(-n2c3ccccc3c3ccccc32)c1 ; N#Cc1ccc(-n2c3ccccc3c3ccccc32)cc1-c1ccccc1-c1c2ccccc2c(-c2ccccc2)c2ccccc12 ; N#Cc1cc(-c2ccccc2-c2c3ccccc3c(-c3ccccc3)c3ccccc23)cc(-n2c3ccccc3c3ccccc32)c1]

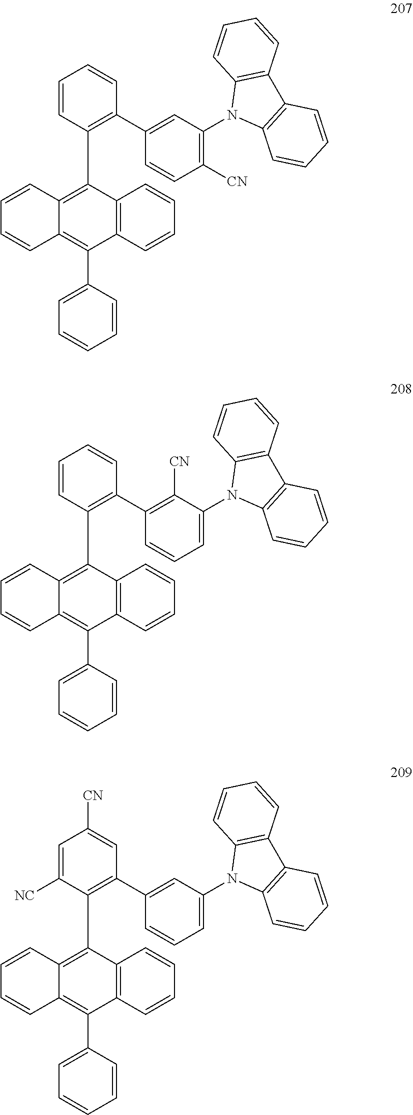 [SMILES: N#Cc1ccc(-c2ccccc2-c2c3ccccc3c(-c3ccccc3)c3ccccc23)cc1-n1c2ccccc2c2ccccc21 ; N#Cc1c(-c2ccccc2-c2c3ccccc3c(-c3ccccc3)c3ccccc23)cccc1-n1c2ccccc2c2ccccc21 ; N#Cc1cc(C#N)c(-c2c3ccccc3c(-c3ccccc3)c3ccccc23)c(-c2cccc(-n3c4ccccc4c4ccccc43)c2)c1]

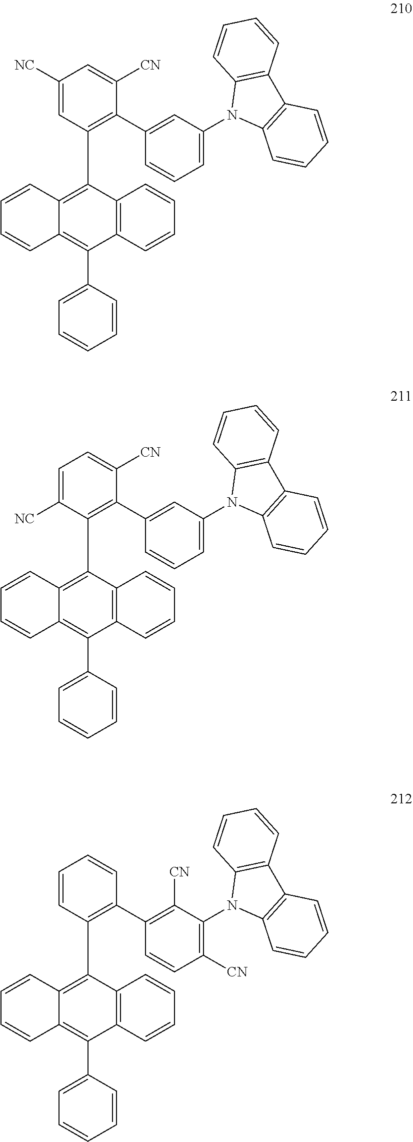 [SMILES: N#Cc1cc(C#N)c(-c2cccc(-n3c4ccccc4c4ccccc43)c2)c(-c2c3ccccc3c(-c3ccccc3)c3ccccc23)c1 ; N#Cc1ccc(C#N)c(-c2c3ccccc3c(-c3ccccc3)c3ccccc23)c1-c1cccc(-n2c3ccccc3c3ccccc32)c1 ; N#Cc1ccc(-c2ccccc2-c2c3ccccc3c(-c3ccccc3)c3ccccc23)c(C#N)c1-n1c2ccccc2c2ccccc21]

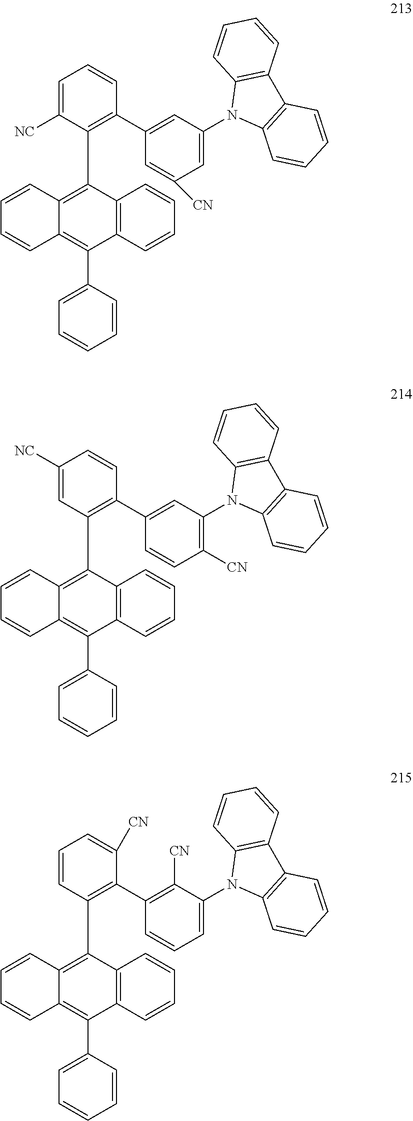 [SMILES: N#Cc1cc(-c2cccc(C#N)c2-c2c3ccccc3c(-c3ccccc3)c3ccccc23)cc(-n2c3ccccc3c3ccccc32)c1 ; N#Cc1ccc(-c2ccc(C#N)c(-n3c4ccccc4c4ccccc43)c2)c(-c2c3ccccc3c(-c3ccccc3)c3ccccc23)c1 ; N#Cc1cccc(-c2c3ccccc3c(-c3ccccc3)c3ccccc23)c1-c1cccc(-n2c3ccccc3c3ccccc32)c1C#N]

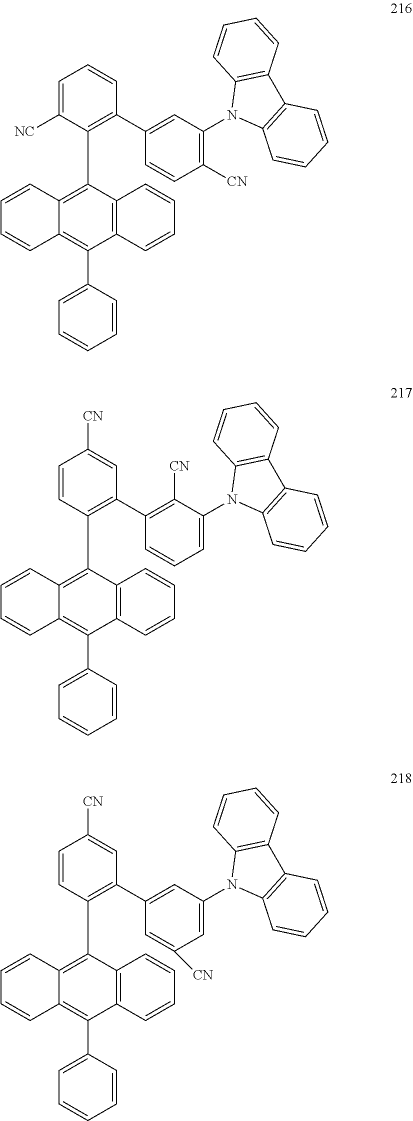 [SMILES: N#Cc1ccc(-c2cccc(C#N)c2-c2c3ccccc3c(-c3ccccc3)c3ccccc23)cc1-n1c2ccccc2c2ccccc21 ; N#Cc1ccc(-c2c3ccccc3c(-c3ccccc3)c3ccccc23)c(-c2cccc(-n3c4ccccc4c4ccccc43)c2C#N)c1 ; N#Cc1cc(-c2cc(C#N)ccc2-c2c3ccccc3c(-c3ccccc3)c3ccccc23)cc(-n2c3ccccc3c3ccccc32)c1]

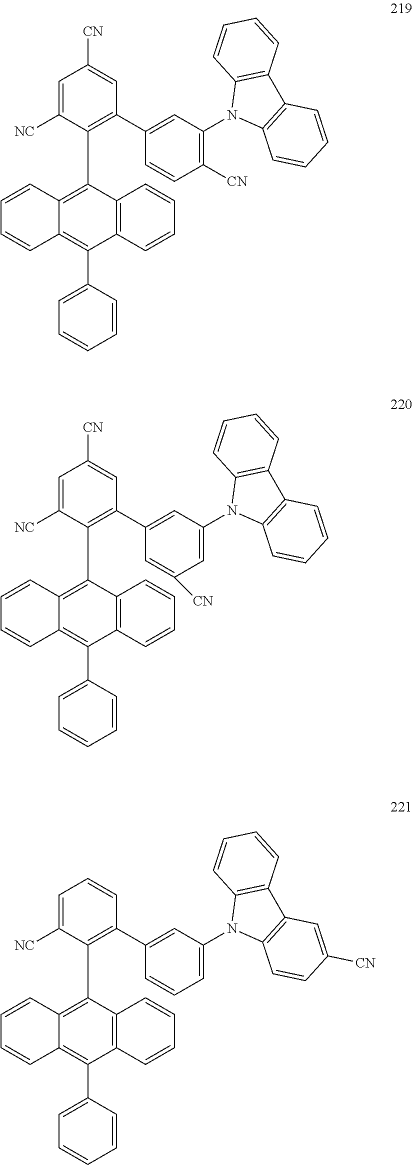 [SMILES: N#Cc1cc(C#N)c(-c2c3ccccc3c(-c3ccccc3)c3ccccc23)c(-c2ccc(C#N)c(-n3c4ccccc4c4ccccc43)c2)c1 ; N#Cc1cc(-c2cc(C#N)cc(C#N)c2-c2c3ccccc3c(-c3ccccc3)c3ccccc23)cc(-n2c3ccccc3c3ccccc32)c1 ; N#Cc1ccc2c(c1)c1ccccc1n2-c1cccc(-c2cccc(C#N)c2-c2c3ccccc3c(-c3ccccc3)c3ccccc23)c1]

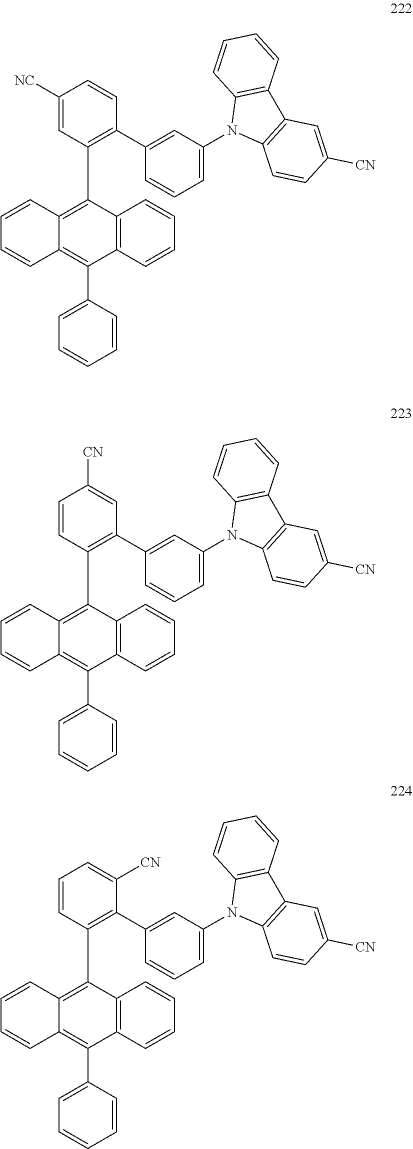 [SMILES: N#Cc1ccc(-c2cccc(-n3c4ccccc4c4cc(C#N)ccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccccc4c4cc(C#N)ccc43)c2)c1 ; N#Cc1ccc2c(c1)c1ccccc1n2-c1cccc(-c2c(C#N)cccc2-c2c3ccccc3c(-c3ccccc3)c3ccccc23)c1]

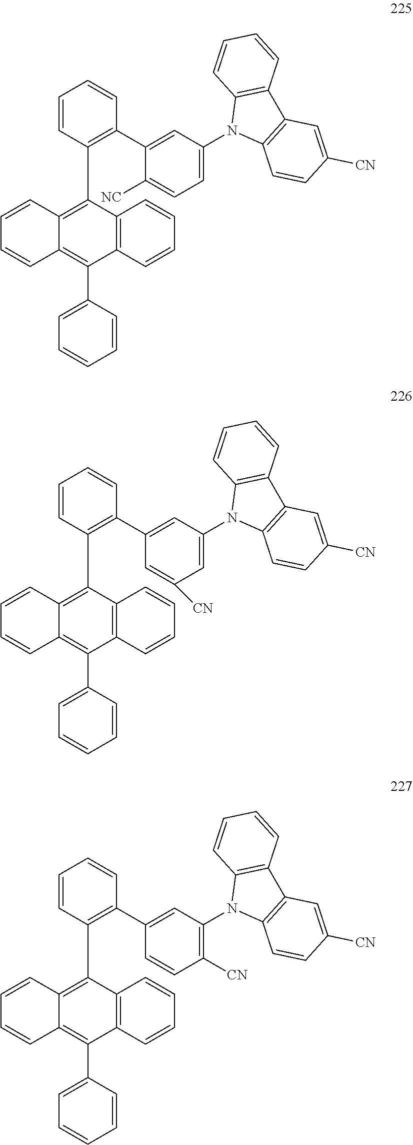 [SMILES: N#Cc1ccc2c(c1)c1ccccc1n2-c1ccc(C#N)c(-c2ccccc2-c2c3ccccc3c(-c3ccccc3)c3ccccc23)c1 ; N#Cc1cc(-c2ccccc2-c2c3ccccc3c(-c3ccccc3)c3ccccc23)cc(-n2c3ccccc3c3cc(C#N)ccc32)c1 ; N#Cc1ccc2c(c1)c1ccccc1n2-c1cc(-c2ccccc2-c2c3ccccc3c(-c3ccccc3)c3ccccc23)ccc1C#N]

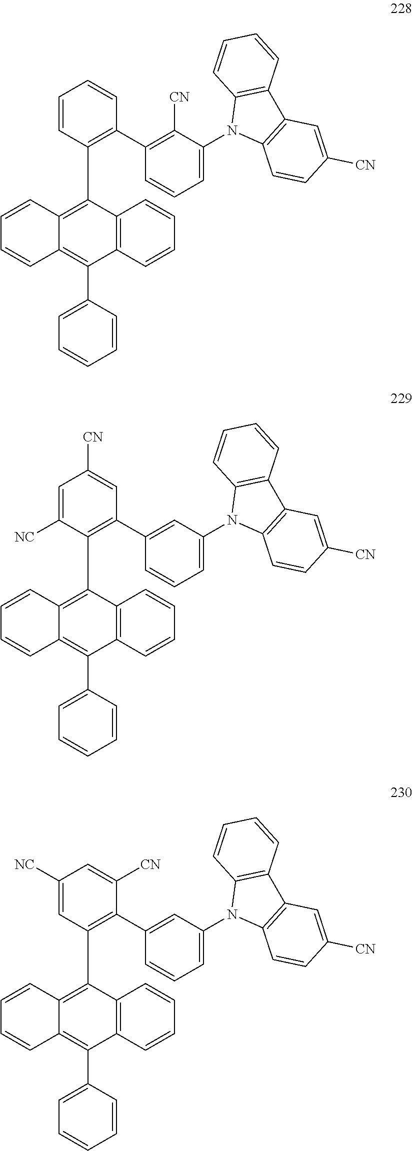 [SMILES: N#Cc1ccc2c(c1)c1ccccc1n2-c1cccc(-c2ccccc2-c2c3ccccc3c(-c3ccccc3)c3ccccc23)c1C#N ; N#Cc1cc(C#N)c(-c2c3ccccc3c(-c3ccccc3)c3ccccc23)c(-c2cccc(-n3c4ccccc4c4cc(C#N)ccc43)c2)c1 ; N#Cc1cc(C#N)c(-c2cccc(-n3c4ccccc4c4cc(C#N)ccc43)c2)c(-c2c3ccccc3c(-c3ccccc3)c3ccccc23)c1]

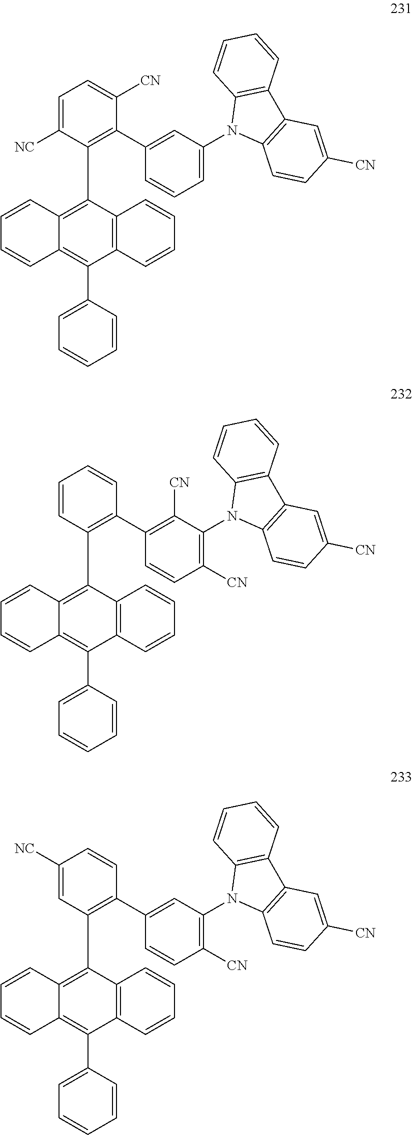 [SMILES: N#Cc1ccc2c(c1)c1ccccc1n2-c1cccc(-c2c(C#N)ccc(C#N)c2-c2c3ccccc3c(-c3ccccc3)c3ccccc23)c1 ; N#Cc1ccc2c(c1)c1ccccc1n2-c1c(C#N)ccc(-c2ccccc2-c2c3ccccc3c(-c3ccccc3)c3ccccc23)c1C#N ; N#Cc1ccc(-c2ccc(C#N)c(-n3c4ccccc4c4cc(C#N)ccc43)c2)c(-c2c3ccccc3c(-c3ccccc3)c3ccccc23)c1]

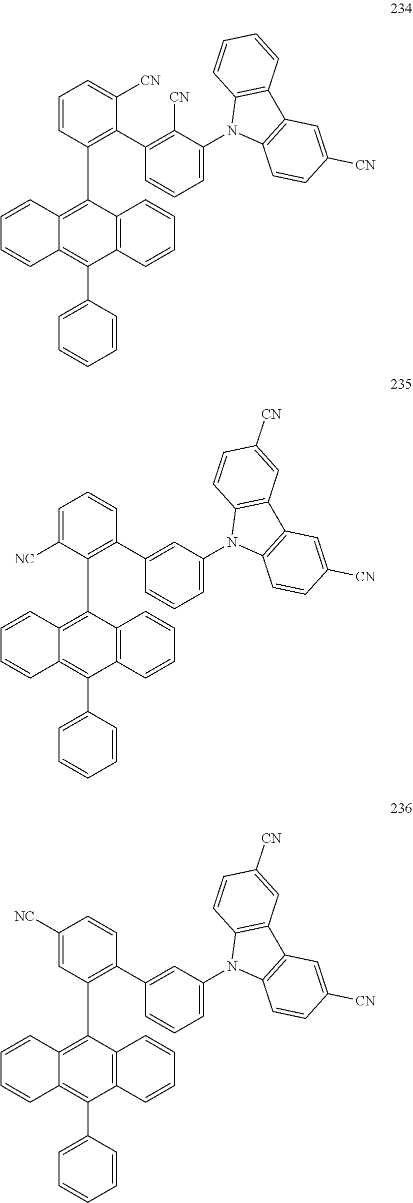 [SMILES: N#Cc1ccc2c(c1)c1ccccc1n2-c1cccc(-c2c(C#N)cccc2-c2c3ccccc3c(-c3ccccc3)c3ccccc23)c1C#N ; N#Cc1ccc2c(c1)c1cc(C#N)ccc1n2-c1cccc(-c2cccc(C#N)c2-c2c3ccccc3c(-c3ccccc3)c3ccccc23)c1 ; N#Cc1ccc(-c2cccc(-n3c4ccc(C#N)cc4c4cc(C#N)ccc43)c2)c(-c2c3ccccc3c(-c3ccccc3)c3ccccc23)c1]

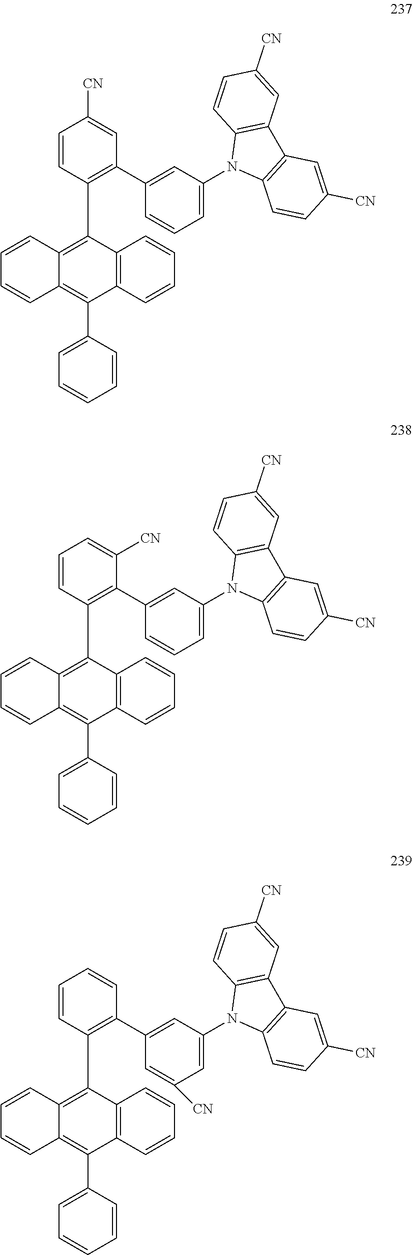 [SMILES: N#Cc1ccc(-c2c3ccccc3c(-c3ccccc3)c3ccccc23)c(-c2cccc(-n3c4ccc(C#N)cc4c4cc(C#N)ccc43)c2)c1 ; N#Cc1ccc2c(c1)c1cc(C#N)ccc1n2-c1cccc(-c2c(C#N)cccc2-c2c3ccccc3c(-c3ccccc3)c3ccccc23)c1 ; N#Cc1cc(-c2ccccc2-c2c3ccccc3c(-c3ccccc3)c3ccccc23)cc(-n2c3ccc(C#N)cc3c3cc(C#N)ccc32)c1]

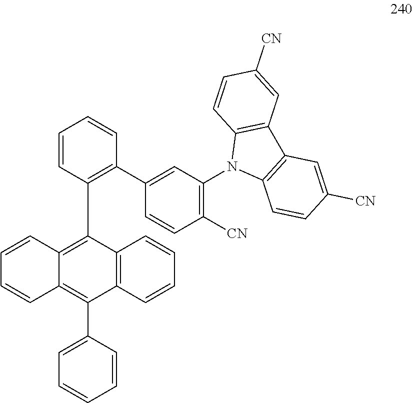 [SMILES: N#Cc1ccc2c(c1)c1cc(C#N)ccc1n2-c1cc(-c2ccccc2-c2c3ccccc3c(-c3ccccc3)c3ccccc23)ccc1C#N]

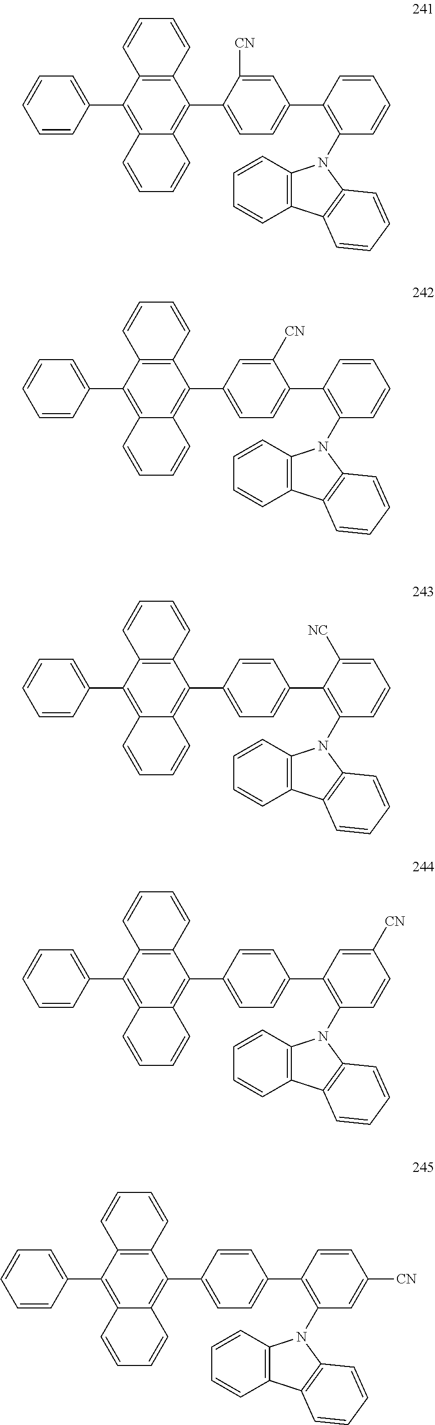 [SMILES: N#Cc1cc(-c2ccccc2-n2c3ccccc3c3ccccc32)ccc1-c1c2ccccc2c(-c2ccccc2)c2ccccc12 ; N#Cc1cc(-c2c3ccccc3c(-c3ccccc3)c3ccccc23)ccc1-c1ccccc1-n1c2ccccc2c2ccccc21 ; N#Cc1cccc(-n2c3ccccc3c3ccccc32)c1-c1ccc(-c2c3ccccc3c(-c3ccccc3)c3ccccc23)cc1 ; N#Cc1ccc(-n2c3ccccc3c3ccccc32)c(-c2ccc(-c3c4ccccc4c(-c4ccccc4)c4ccccc34)cc2)c1 ; N#Cc1ccc(-c2ccc(-c3c4ccccc4c(-c4ccccc4)c4ccccc34)cc2)c(-n2c3ccccc3c3ccccc32)c1]

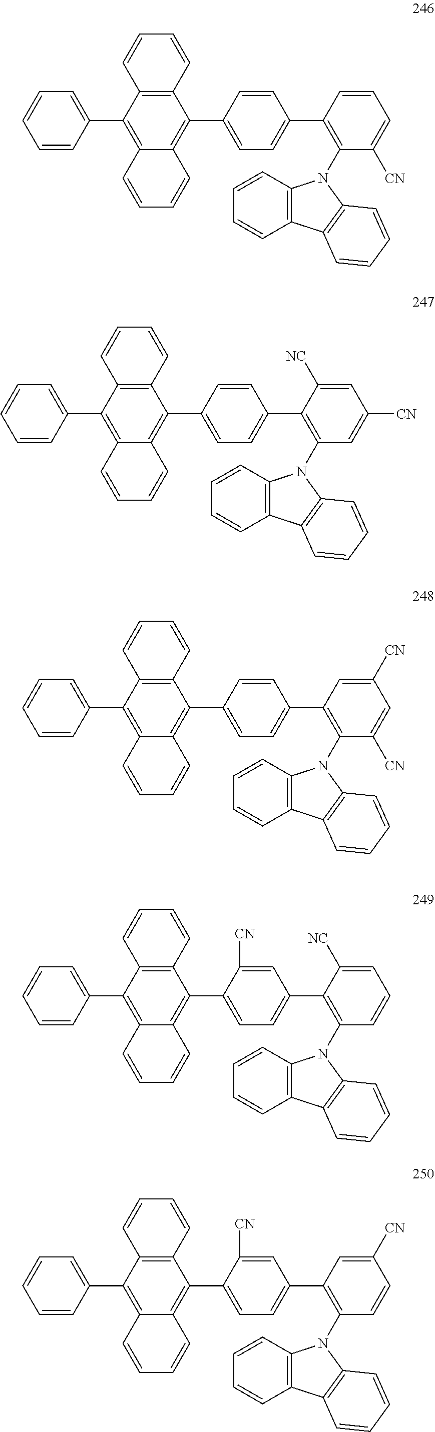 [SMILES: N#Cc1cccc(-c2ccc(-c3c4ccccc4c(-c4ccccc4)c4ccccc34)cc2)c1-n1c2ccccc2c2ccccc21 ; N#Cc1cc(C#N)c(-c2ccc(-c3c4ccccc4c(-c4ccccc4)c4ccccc34)cc2)c(-n2c3ccccc3c3ccccc32)c1 ; N#Cc1cc(C#N)c(-n2c3ccccc3c3ccccc32)c(-c2ccc(-c3c4ccccc4c(-c4ccccc4)c4ccccc34)cc2)c1 ; N#Cc1cc(-c2c(C#N)cccc2-n2c3ccccc3c3ccccc32)ccc1-c1c2ccccc2c(-c2ccccc2)c2ccccc12 ; N#Cc1ccc(-n2c3ccccc3c3ccccc32)c(-c2ccc(-c3c4ccccc4c(-c4ccccc4)c4ccccc34)c(C#N)c2)c1]

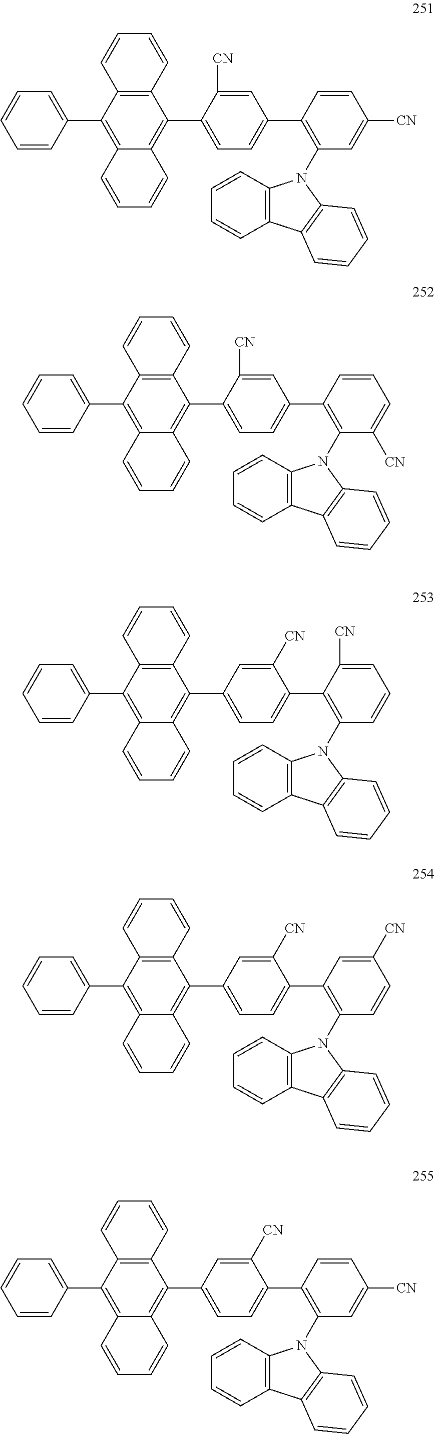 [SMILES: N#Cc1ccc(-c2ccc(-c3c4ccccc4c(-c4ccccc4)c4ccccc34)c(C#N)c2)c(-n2c3ccccc3c3ccccc32)c1 ; N#Cc1cc(-c2cccc(C#N)c2-n2c3ccccc3c3ccccc32)ccc1-c1c2ccccc2c(-c2ccccc2)c2ccccc12 ; N#Cc1cc(-c2c3ccccc3c(-c3ccccc3)c3ccccc23)ccc1-c1c(C#N)cccc1-n1c2ccccc2c2ccccc21 ; N#Cc1ccc(-n2c3ccccc3c3ccccc32)c(-c2ccc(-c3c4ccccc4c(-c4ccccc4)c4ccccc34)cc2C#N)c1 ; N#Cc1ccc(-c2ccc(-c3c4ccccc4c(-c4ccccc4)c4ccccc34)cc2C#N)c(-n2c3ccccc3c3ccccc32)c1]

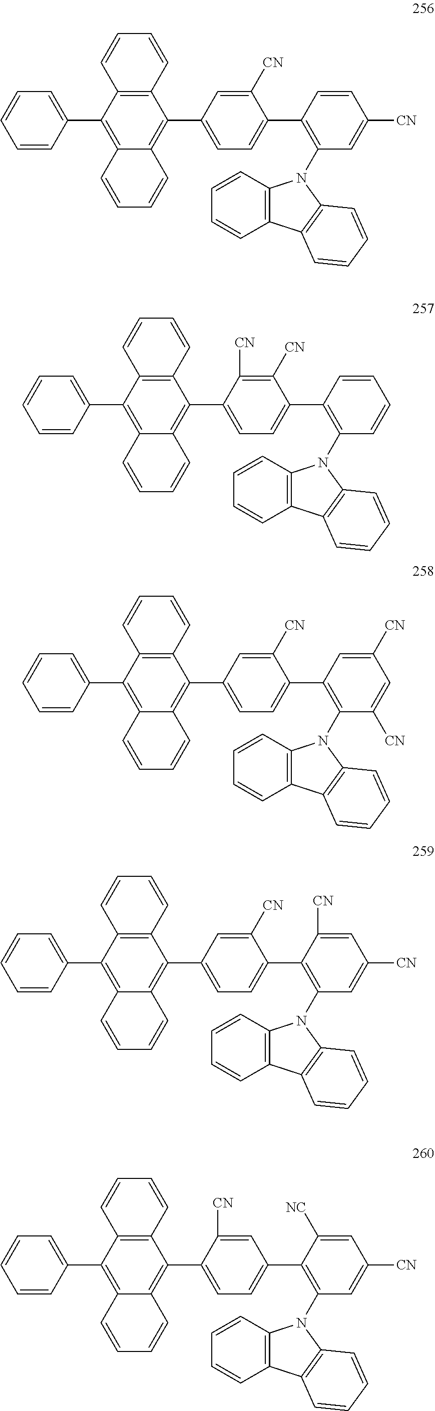 [SMILES: N#Cc1ccc(-c2ccc(-c3c4ccccc4c(-c4ccccc4)c4ccccc34)cc2C#N)c(-n2c3ccccc3c3ccccc32)c1 ; N#Cc1c(-c2ccccc2-n2c3ccccc3c3ccccc32)ccc(-c2c3ccccc3c(-c3ccccc3)c3ccccc23)c1C#N ; N#Cc1cc(C#N)c(-n2c3ccccc3c3ccccc32)c(-c2ccc(-c3c4ccccc4c(-c4ccccc4)c4ccccc34)cc2C#N)c1 ; N#Cc1cc(C#N)c(-c2ccc(-c3c4ccccc4c(-c4ccccc4)c4ccccc34)cc2C#N)c(-n2c3ccccc3c3ccccc32)c1 ; N#Cc1cc(C#N)c(-c2ccc(-c3c4ccccc4c(-c4ccccc4)c4ccccc34)c(C#N)c2)c(-n2c3ccccc3c3ccccc32)c1]

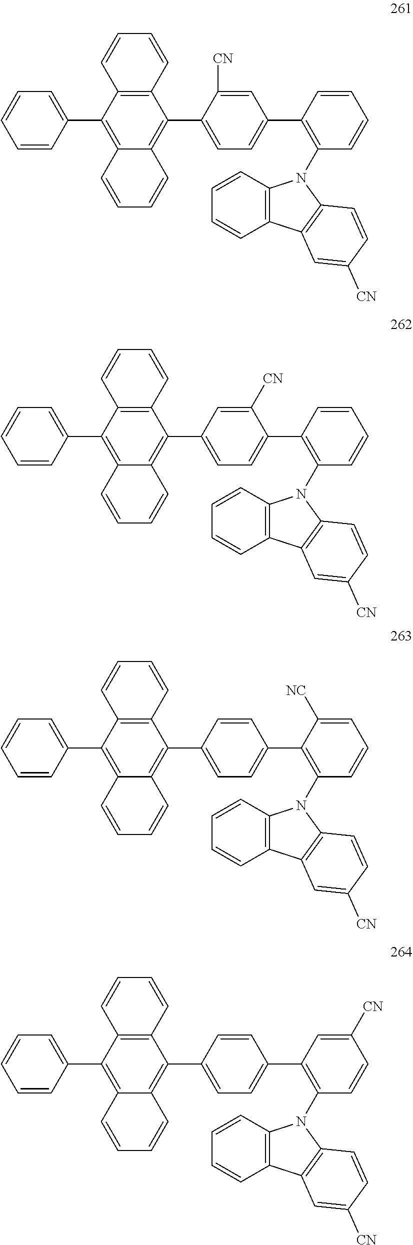 [SMILES: N#Cc1ccc2c(c1)c1ccccc1n2-c1ccccc1-c1ccc(-c2c3ccccc3c(-c3ccccc3)c3ccccc23)c(C#N)c1 ; N#Cc1ccc2c(c1)c1ccccc1n2-c1ccccc1-c1ccc(-c2c3ccccc3c(-c3ccccc3)c3ccccc23)cc1C#N ; N#Cc1ccc2c(c1)c1ccccc1n2-c1cccc(C#N)c1-c1ccc(-c2c3ccccc3c(-c3ccccc3)c3ccccc23)cc1 ; N#Cc1ccc(-n2c3ccccc3c3cc(C#N)ccc32)c(-c2ccc(-c3c4ccccc4c(-c4ccccc4)c4ccccc34)cc2)c1]

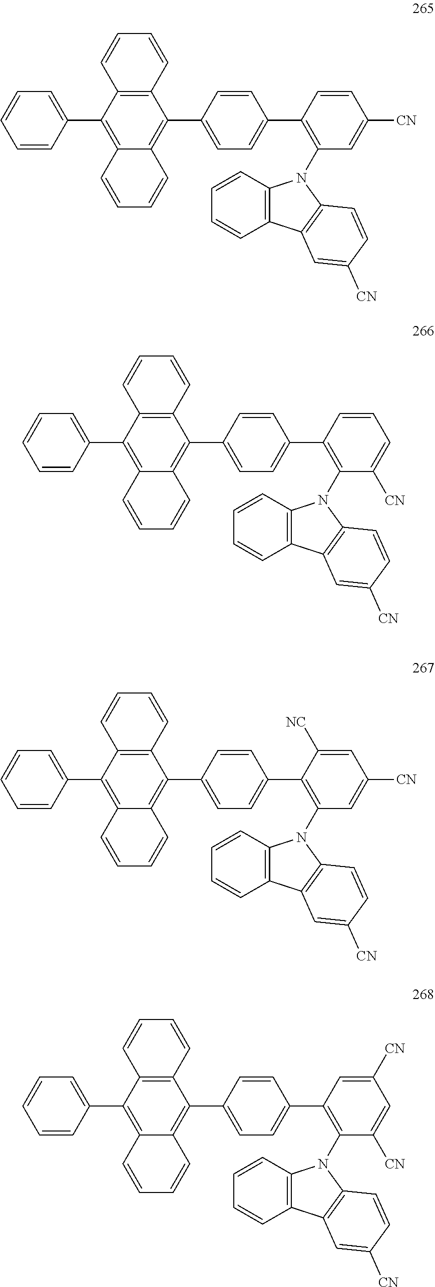 [SMILES: N#Cc1ccc(-c2ccc(-c3c4ccccc4c(-c4ccccc4)c4ccccc34)cc2)c(-n2c3ccccc3c3cc(C#N)ccc32)c1 ; N#Cc1ccc2c(c1)c1ccccc1n2-c1c(C#N)cccc1-c1ccc(-c2c3ccccc3c(-c3ccccc3)c3ccccc23)cc1 ; N#Cc1cc(C#N)c(-c2ccc(-c3c4ccccc4c(-c4ccccc4)c4ccccc34)cc2)c(-n2c3ccccc3c3cc(C#N)ccc32)c1 ; N#Cc1cc(C#N)c(-n2c3ccccc3c3cc(C#N)ccc32)c(-c2ccc(-c3c4ccccc4c(-c4ccccc4)c4ccccc34)cc2)c1]

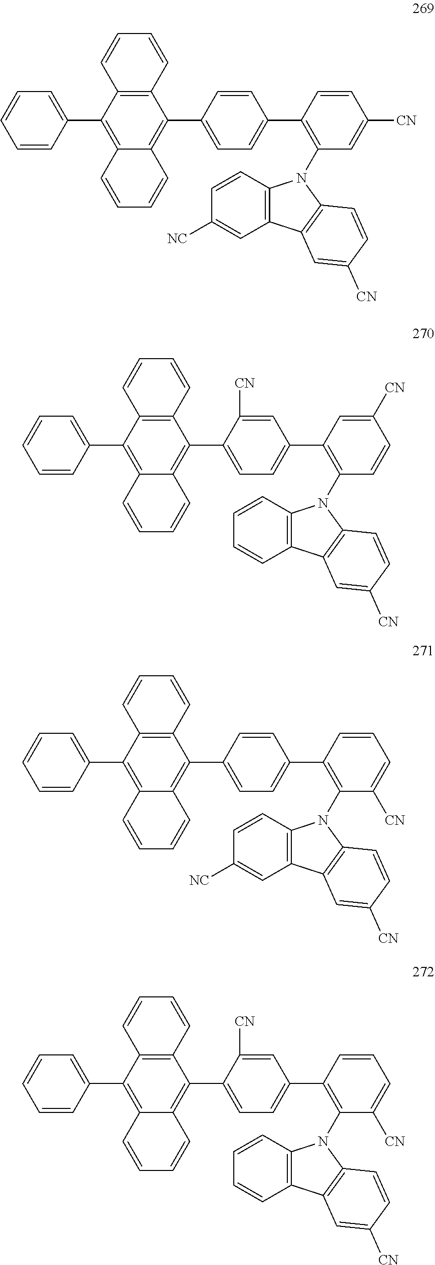 [SMILES: N#Cc1ccc(-c2ccc(-c3c4ccccc4c(-c4ccccc4)c4ccccc34)cc2)c(-n2c3ccc(C#N)cc3c3cc(C#N)ccc32)c1 ; N#Cc1ccc(-n2c3ccccc3c3cc(C#N)ccc32)c(-c2ccc(-c3c4ccccc4c(-c4ccccc4)c4ccccc34)c(C#N)c2)c1 ; N#Cc1ccc2c(c1)c1cc(C#N)ccc1n2-c1c(C#N)cccc1-c1ccc(-c2c3ccccc3c(-c3ccccc3)c3ccccc23)cc1 ; N#Cc1ccc2c(c1)c1ccccc1n2-c1c(C#N)cccc1-c1ccc(-c2c3ccccc3c(-c3ccccc3)c3ccccc23)c(C#N)c1]

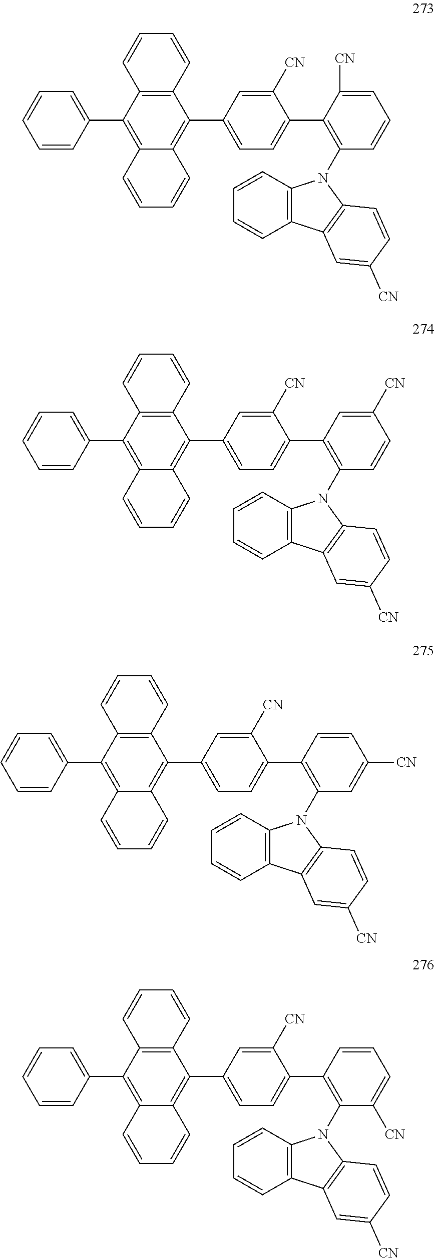 [SMILES: N#Cc1ccc2c(c1)c1ccccc1n2-c1cccc(C#N)c1-c1ccc(-c2c3ccccc3c(-c3ccccc3)c3ccccc23)cc1C#N ; N#Cc1ccc(-n2c3ccccc3c3cc(C#N)ccc32)c(-c2ccc(-c3c4ccccc4c(-c4ccccc4)c4ccccc34)cc2C#N)c1 ; N#Cc1ccc(-c2ccc(-c3c4ccccc4c(-c4ccccc4)c4ccccc34)cc2C#N)c(-n2c3ccccc3c3cc(C#N)ccc32)c1 ; N#Cc1ccc2c(c1)c1ccccc1n2-c1c(C#N)cccc1-c1ccc(-c2c3ccccc3c(-c3ccccc3)c3ccccc23)cc1C#N]

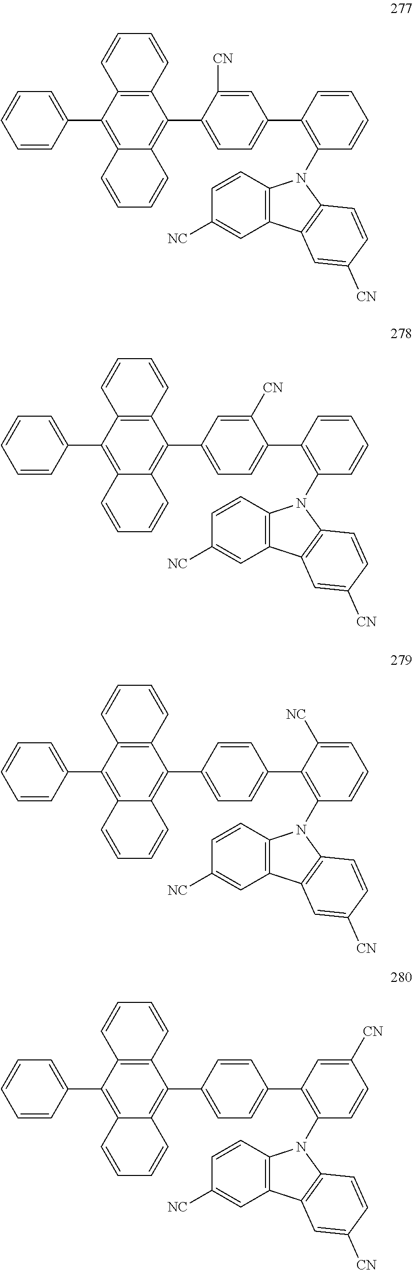 [SMILES: N#Cc1ccc2c(c1)c1cc(C#N)ccc1n2-c1ccccc1-c1ccc(-c2c3ccccc3c(-c3ccccc3)c3ccccc23)c(C#N)c1 ; N#Cc1ccc2c(c1)c1cc(C#N)ccc1n2-c1ccccc1-c1ccc(-c2c3ccccc3c(-c3ccccc3)c3ccccc23)cc1C#N ; N#Cc1ccc2c(c1)c1cc(C#N)ccc1n2-c1cccc(C#N)c1-c1ccc(-c2c3ccccc3c(-c3ccccc3)c3ccccc23)cc1 ; N#Cc1ccc(-n2c3ccc(C#N)cc3c3cc(C#N)ccc32)c(-c2ccc(-c3c4ccccc4c(-c4ccccc4)c4ccccc34)cc2)c1]

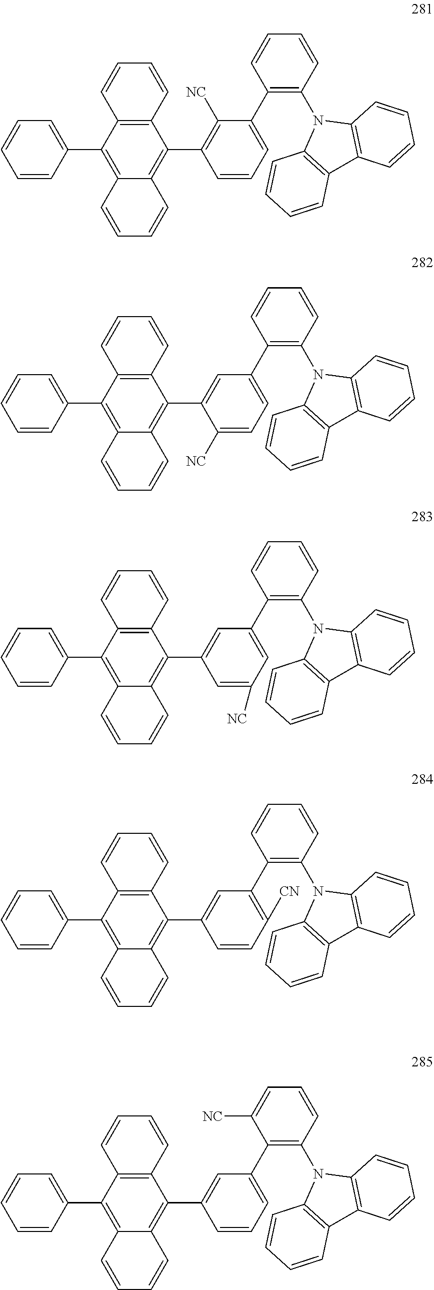 [SMILES: N#Cc1c(-c2ccccc2-n2c3ccccc3c3ccccc32)cccc1-c1c2ccccc2c(-c2ccccc2)c2ccccc12 ; N#Cc1ccc(-c2ccccc2-n2c3ccccc3c3ccccc32)cc1-c1c2ccccc2c(-c2ccccc2)c2ccccc12 ; N#Cc1cc(-c2ccccc2-n2c3ccccc3c3ccccc32)cc(-c2c3ccccc3c(-c3ccccc3)c3ccccc23)c1 ; N#Cc1ccc(-c2c3ccccc3c(-c3ccccc3)c3ccccc23)cc1-c1ccccc1-n1c2ccccc2c2ccccc21 ; N#Cc1cccc(-n2c3ccccc3c3ccccc32)c1-c1cccc(-c2c3ccccc3c(-c3ccccc3)c3ccccc23)c1]

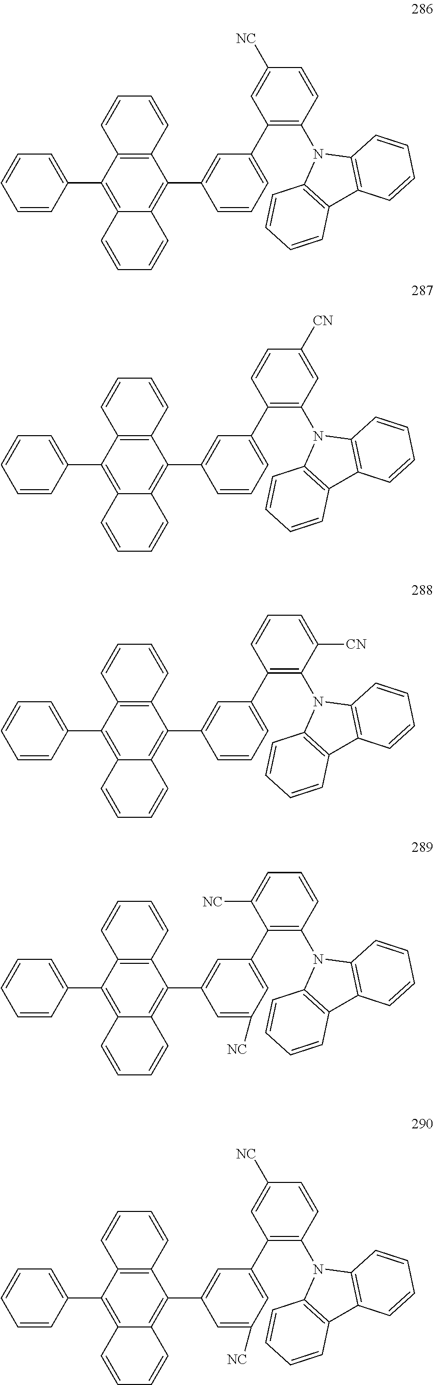 [SMILES: N#Cc1ccc(-n2c3ccccc3c3ccccc32)c(-c2cccc(-c3c4ccccc4c(-c4ccccc4)c4ccccc34)c2)c1 ; N#Cc1ccc(-c2cccc(-c3c4ccccc4c(-c4ccccc4)c4ccccc34)c2)c(-n2c3ccccc3c3ccccc32)c1 ; N#Cc1cccc(-c2cccc(-c3c4ccccc4c(-c4ccccc4)c4ccccc34)c2)c1-n1c2ccccc2c2ccccc21 ; N#Cc1cc(-c2c(C#N)cccc2-n2c3ccccc3c3ccccc32)cc(-c2c3ccccc3c(-c3ccccc3)c3ccccc23)c1 ; N#Cc1cc(-c2cc(C#N)ccc2-n2c3ccccc3c3ccccc32)cc(-c2c3ccccc3c(-c3ccccc3)c3ccccc23)c1]

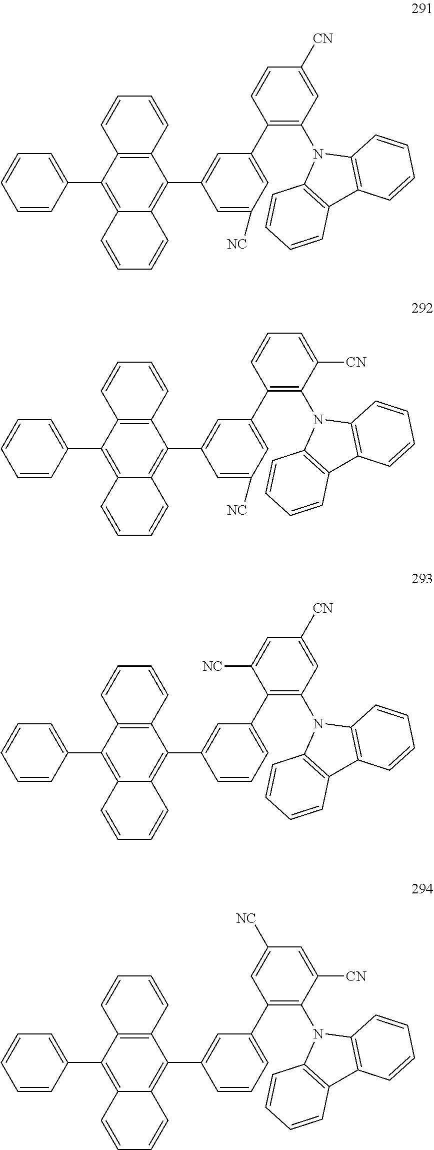 [SMILES: N#Cc1cc(-c2ccc(C#N)cc2-n2c3ccccc3c3ccccc32)cc(-c2c3ccccc3c(-c3ccccc3)c3ccccc23)c1 ; N#Cc1cc(-c2cccc(C#N)c2-n2c3ccccc3c3ccccc32)cc(-c2c3ccccc3c(-c3ccccc3)c3ccccc23)c1 ; N#Cc1cc(C#N)c(-c2cccc(-c3c4ccccc4c(-c4ccccc4)c4ccccc34)c2)c(-n2c3ccccc3c3ccccc32)c1 ; N#Cc1cc(C#N)c(-n2c3ccccc3c3ccccc32)c(-c2cccc(-c3c4ccccc4c(-c4ccccc4)c4ccccc34)c2)c1]

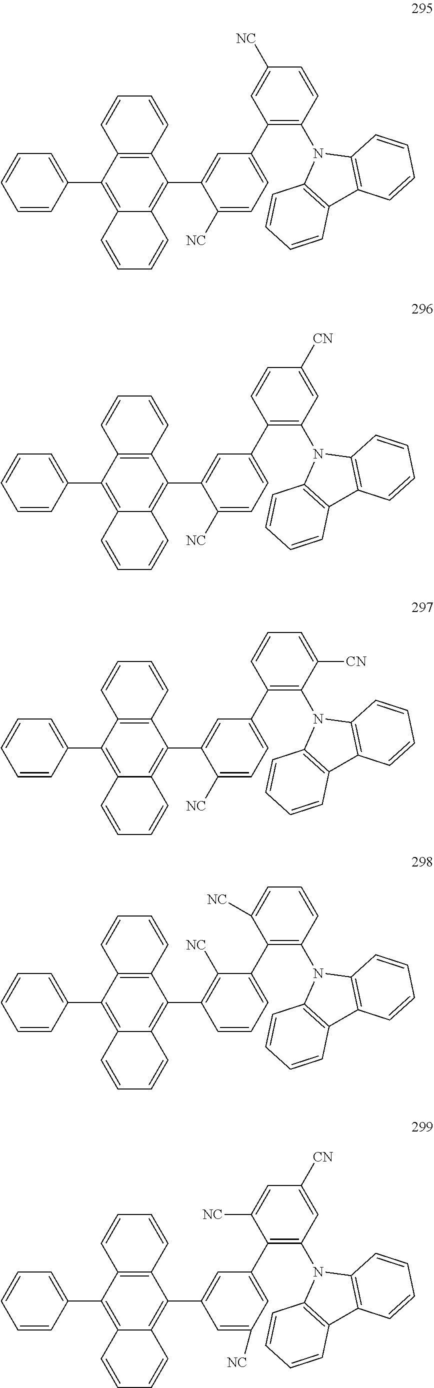 [SMILES: N#Cc1ccc(-n2c3ccccc3c3ccccc32)c(-c2ccc(C#N)c(-c3c4ccccc4c(-c4ccccc4)c4ccccc34)c2)c1 ; N#Cc1ccc(-c2ccc(C#N)c(-c3c4ccccc4c(-c4ccccc4)c4ccccc34)c2)c(-n2c3ccccc3c3ccccc32)c1 ; N#Cc1ccc(-c2cccc(C#N)c2-n2c3ccccc3c3ccccc32)cc1-c1c2ccccc2c(-c2ccccc2)c2ccccc12 ; N#Cc1cccc(-n2c3ccccc3c3ccccc32)c1-c1cccc(-c2c3ccccc3c(-c3ccccc3)c3ccccc23)c1C#N ; N#Cc1cc(-c2c(C#N)cc(C#N)cc2-n2c3ccccc3c3ccccc32)cc(-c2c3ccccc3c(-c3ccccc3)c3ccccc23)c1]

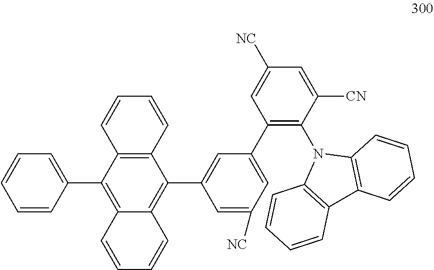 [SMILES: N#Cc1cc(-c2cc(C#N)cc(C#N)c2-n2c3ccccc3c3ccccc32)cc(-c2c3ccccc3c(-c3ccccc3)c3ccccc23)c1]

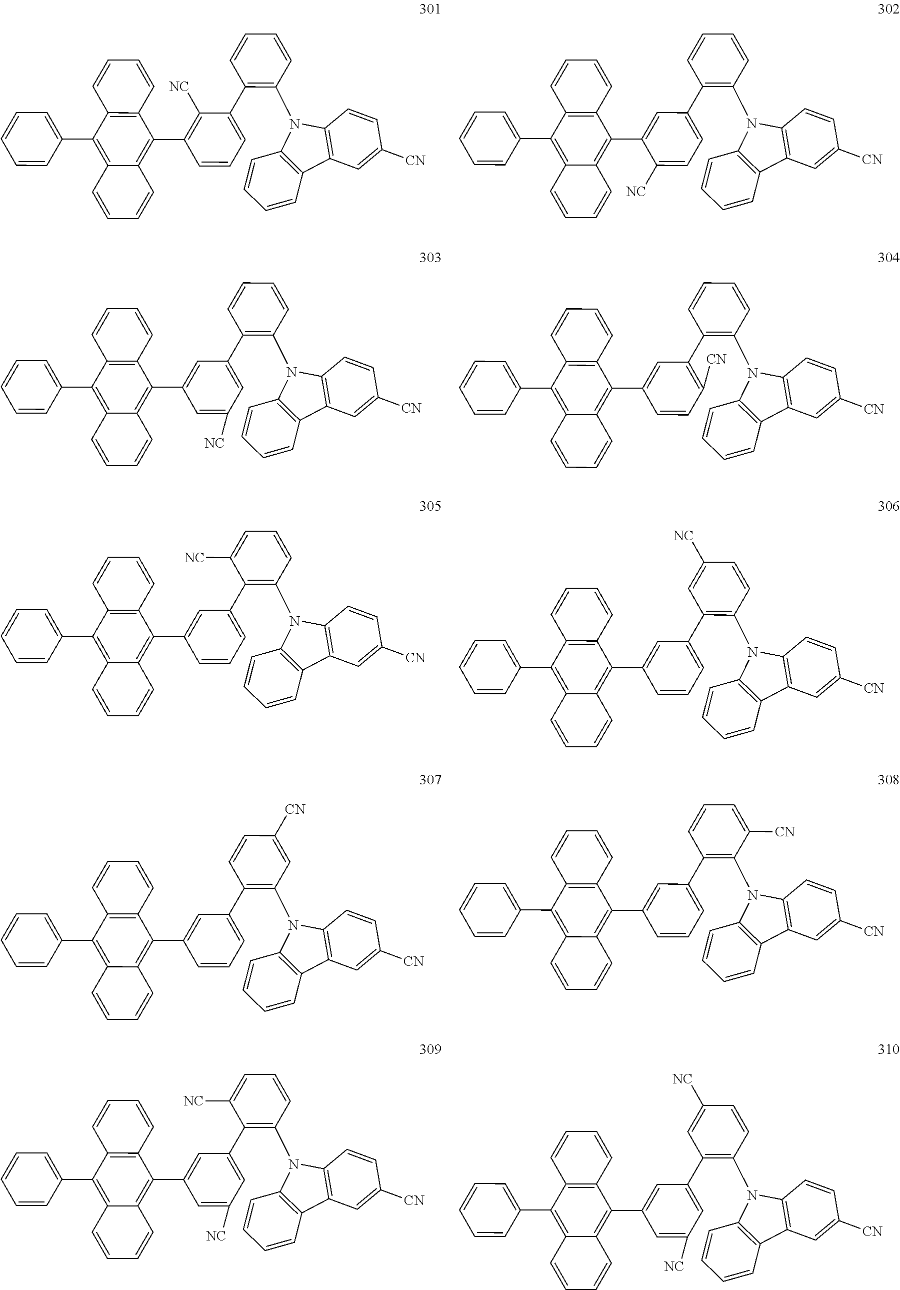

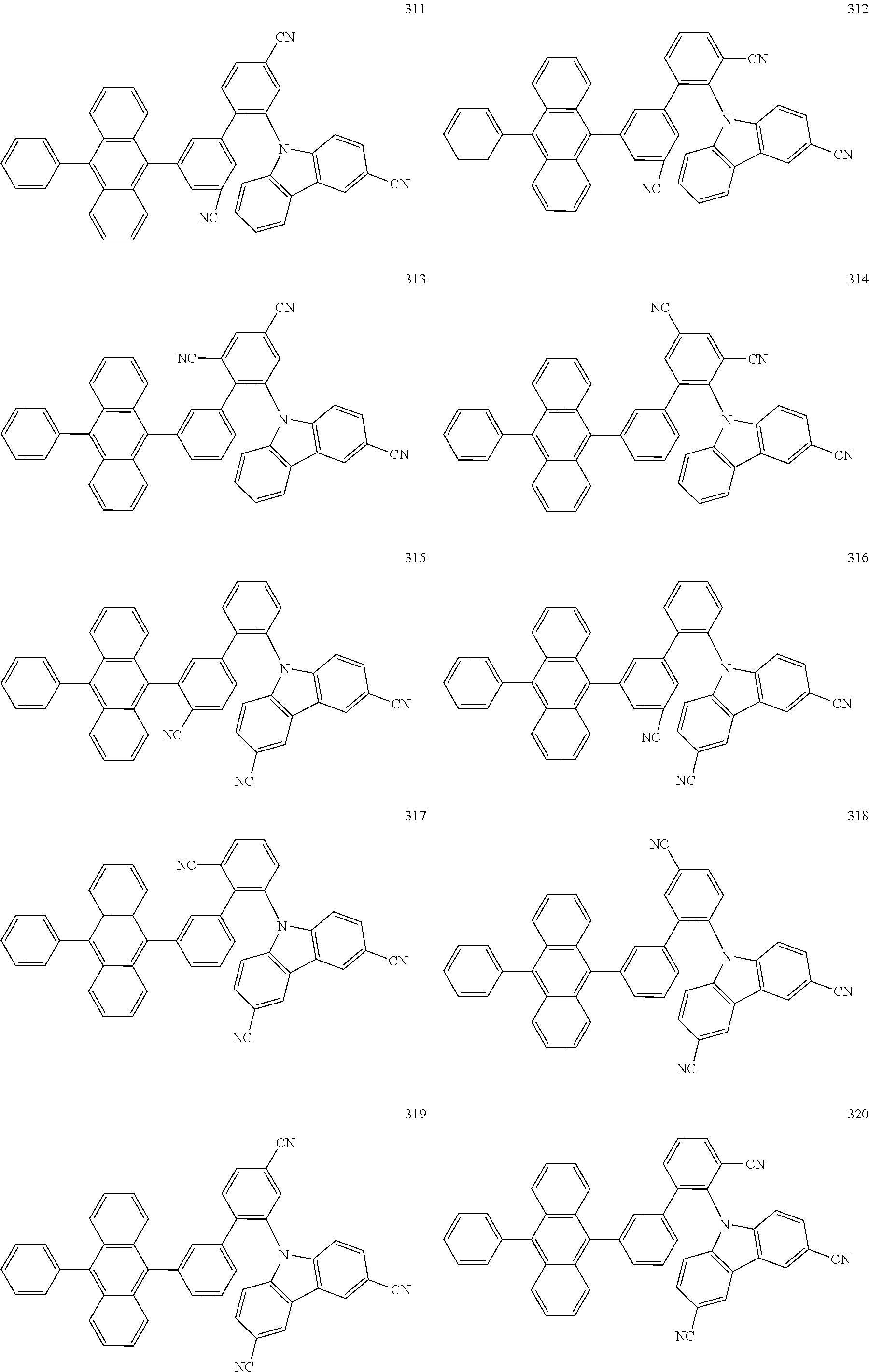

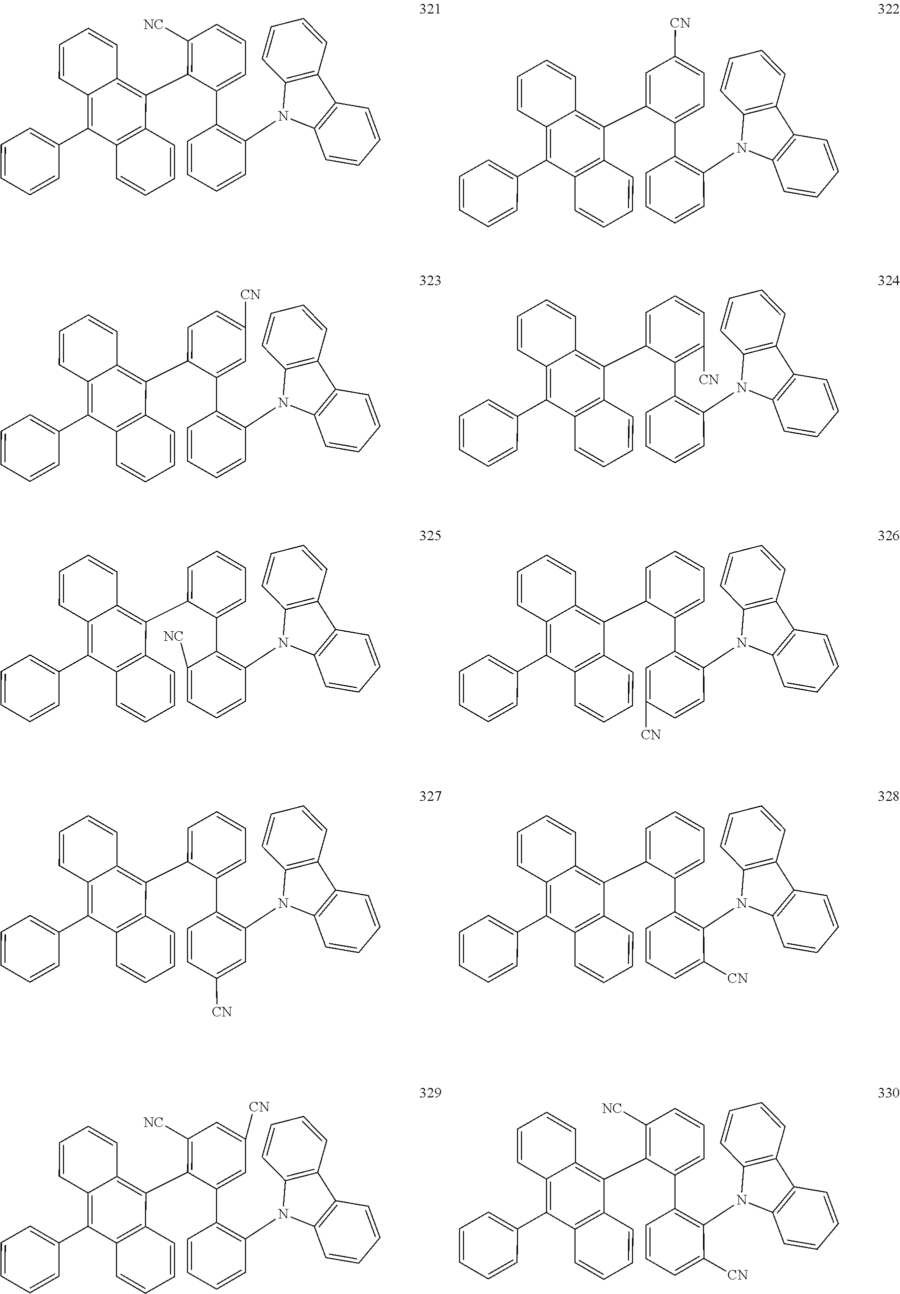

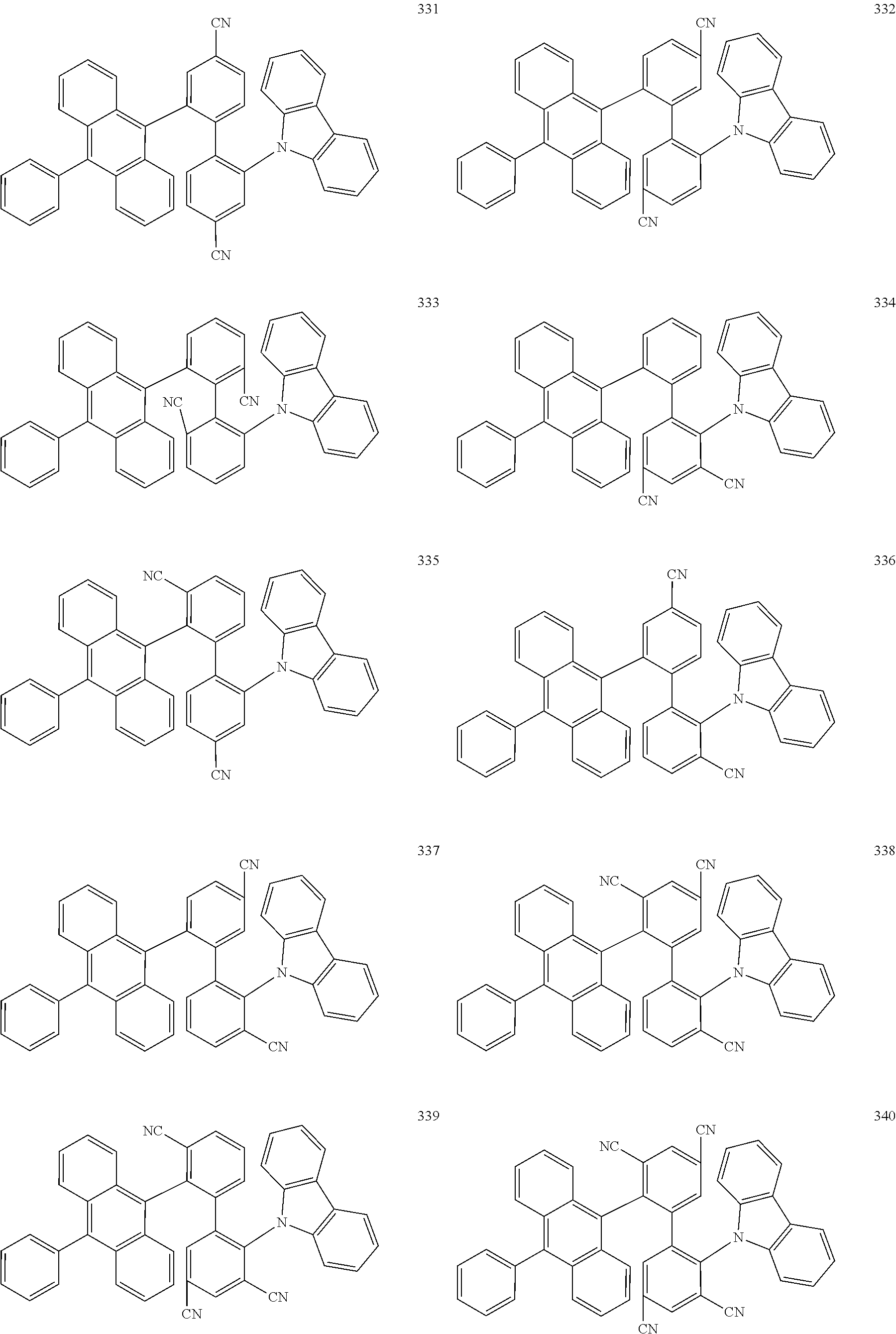

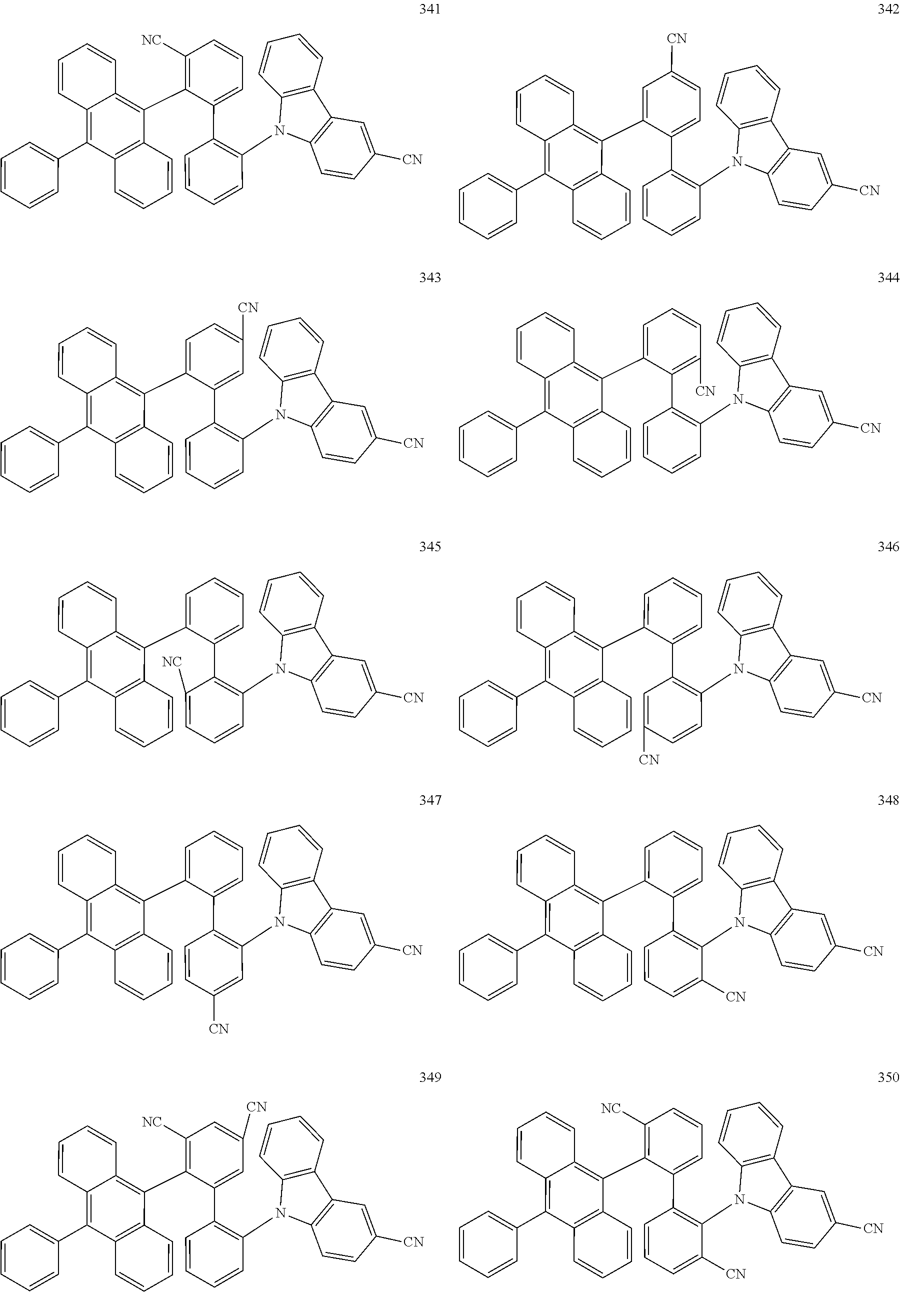

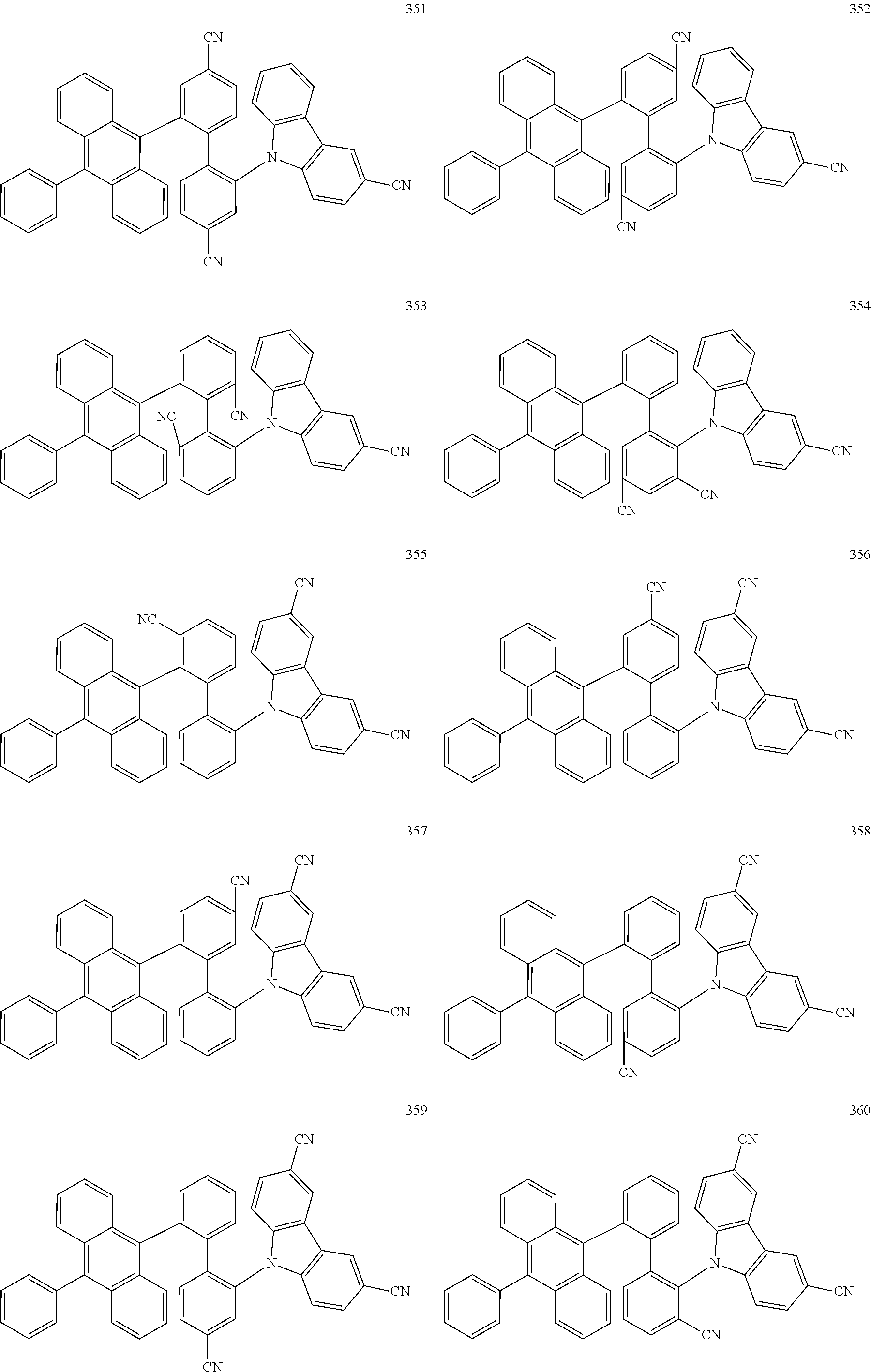

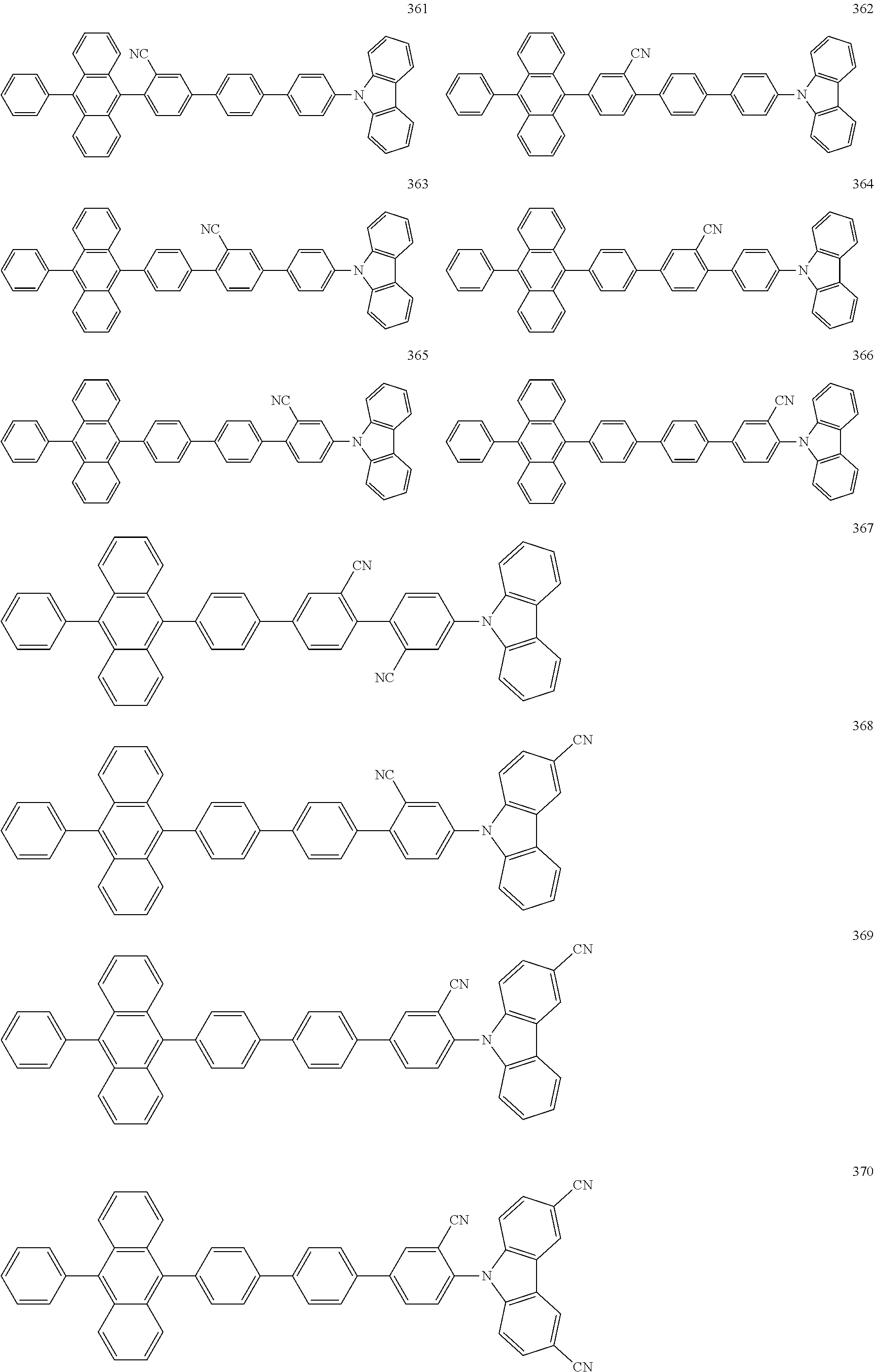

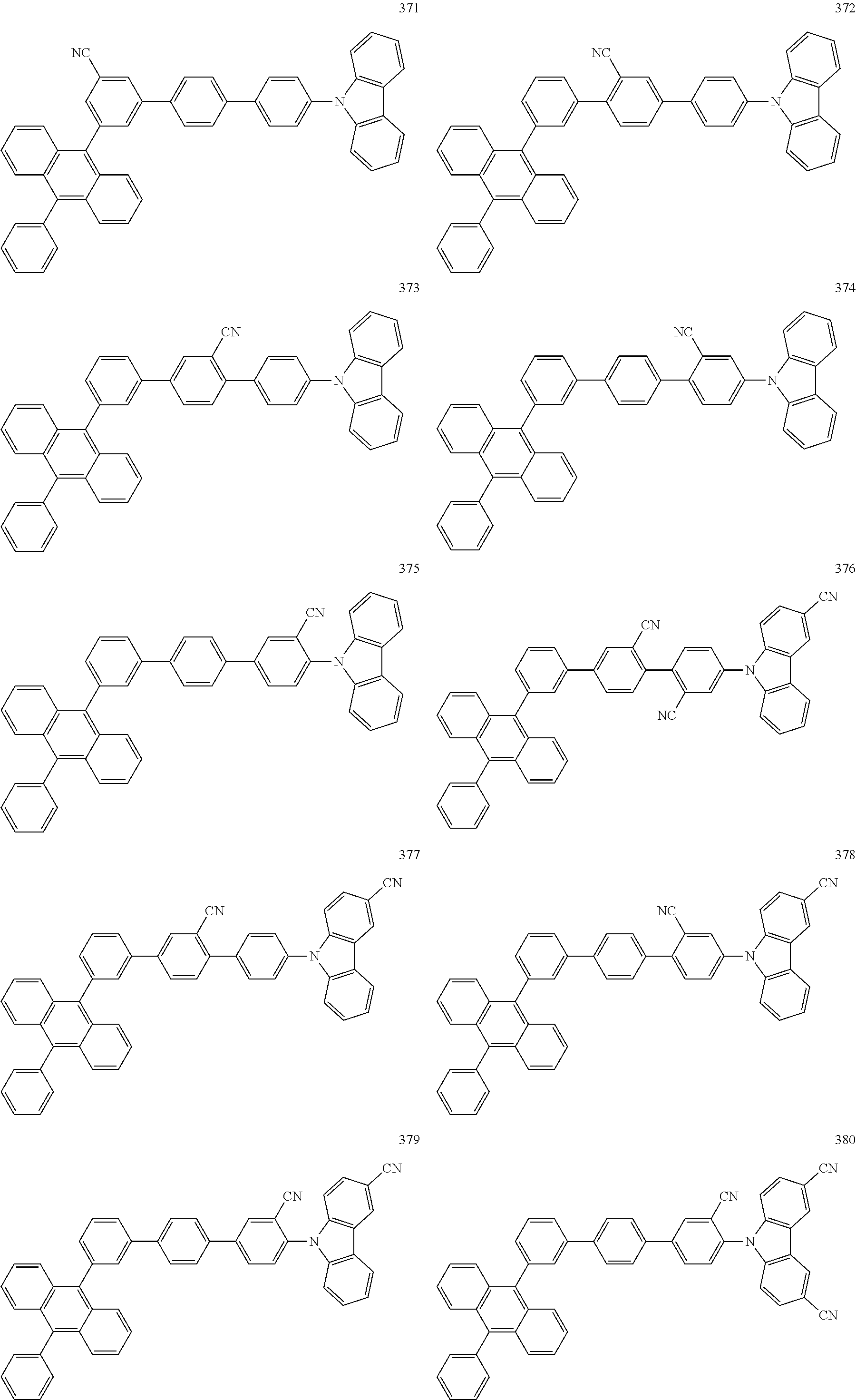

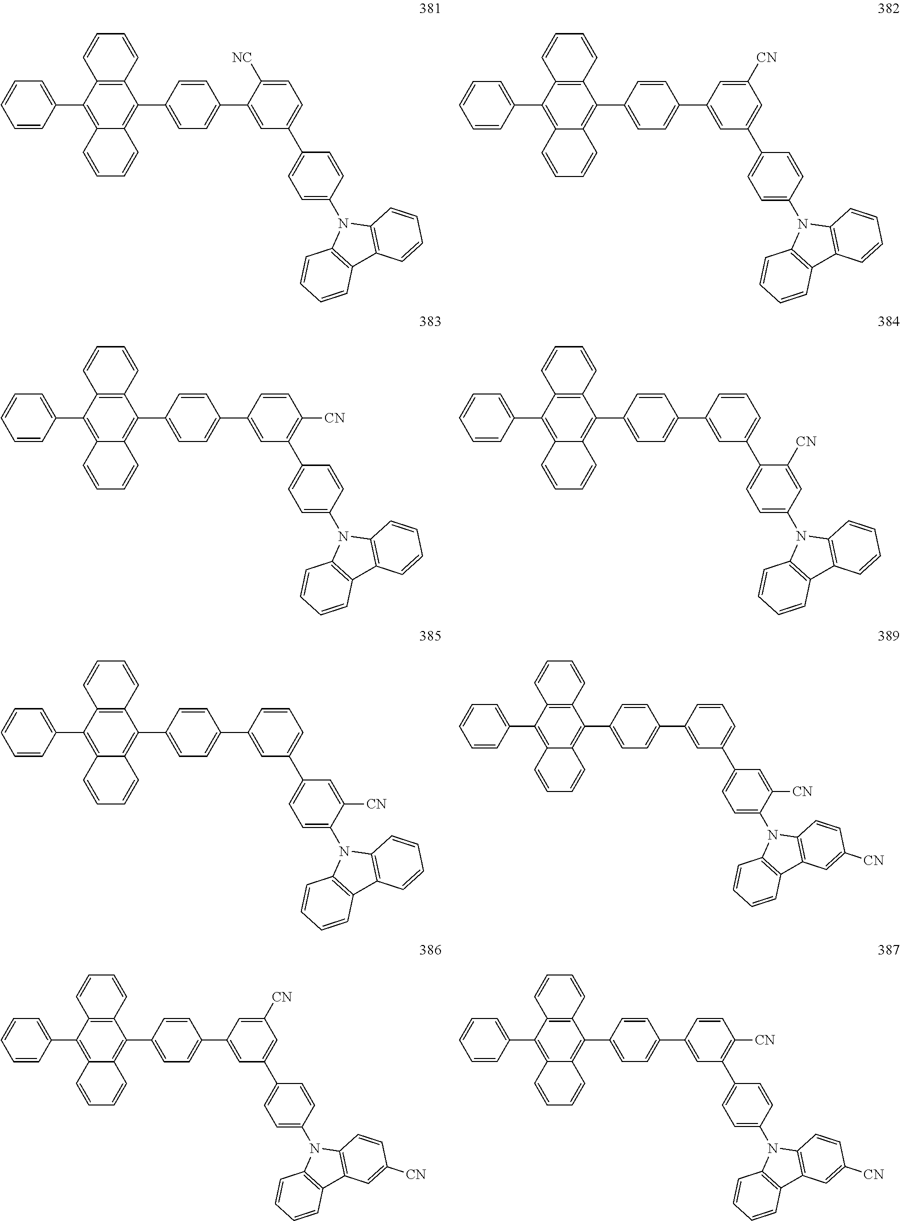

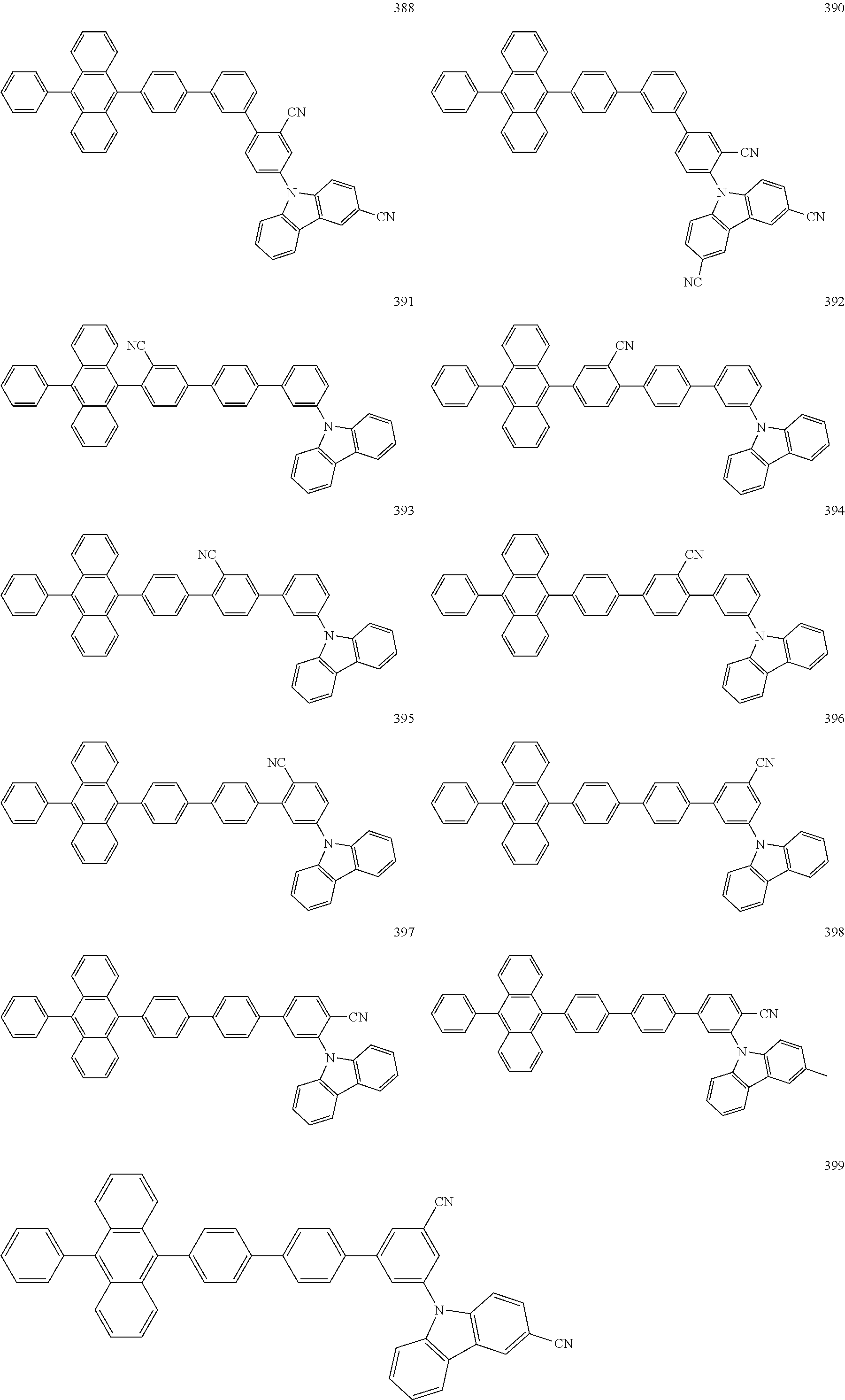

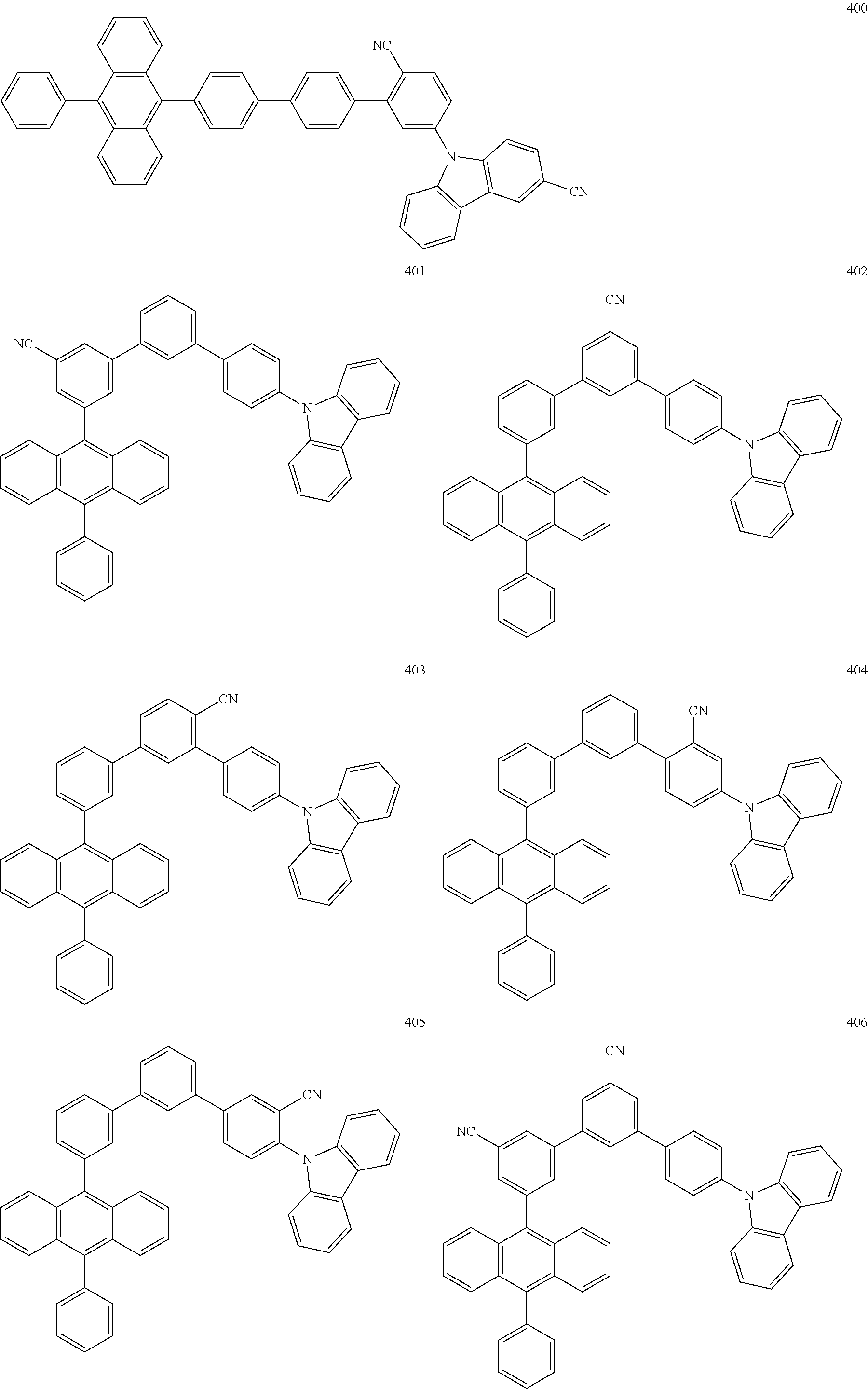

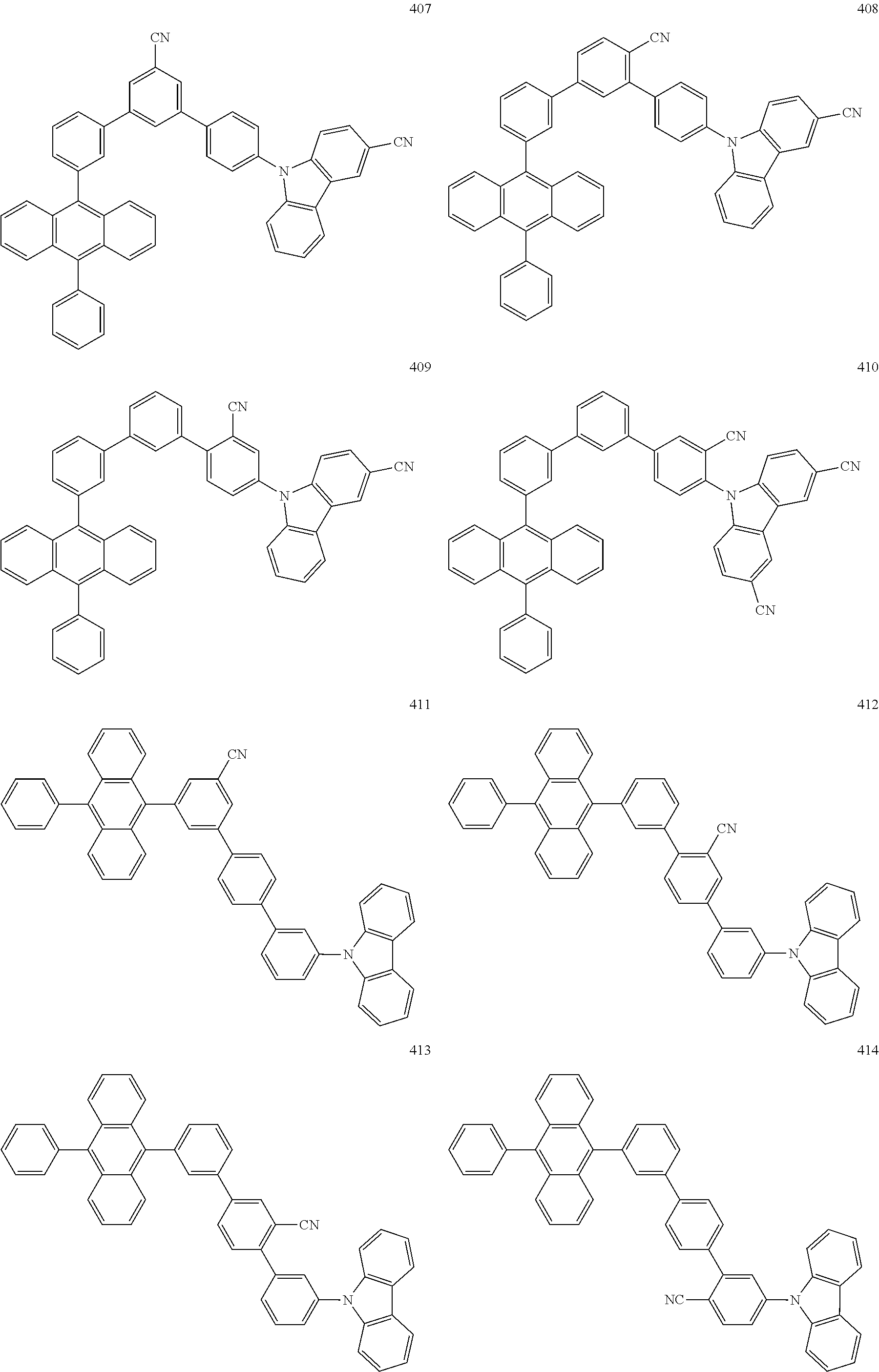

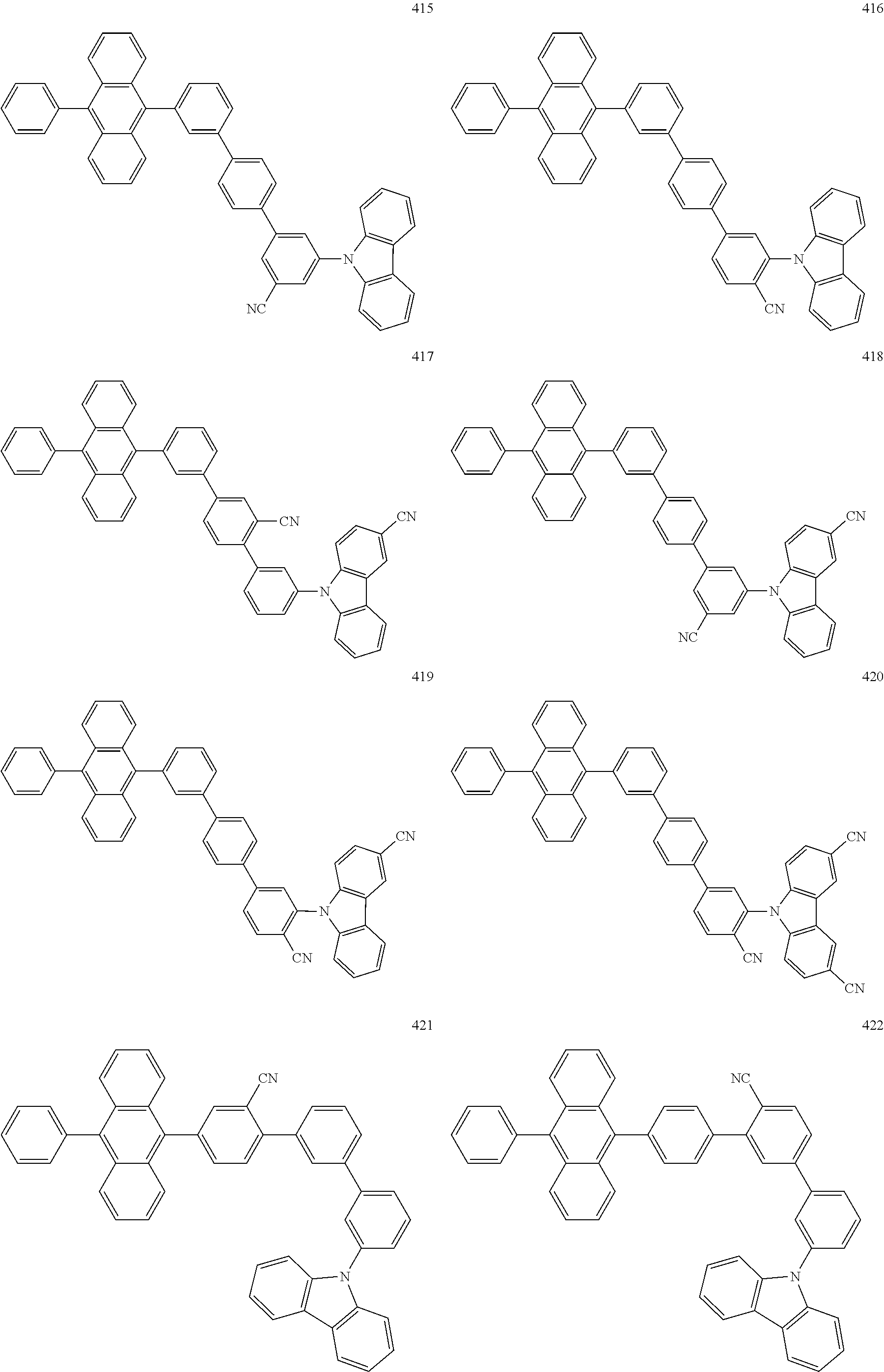

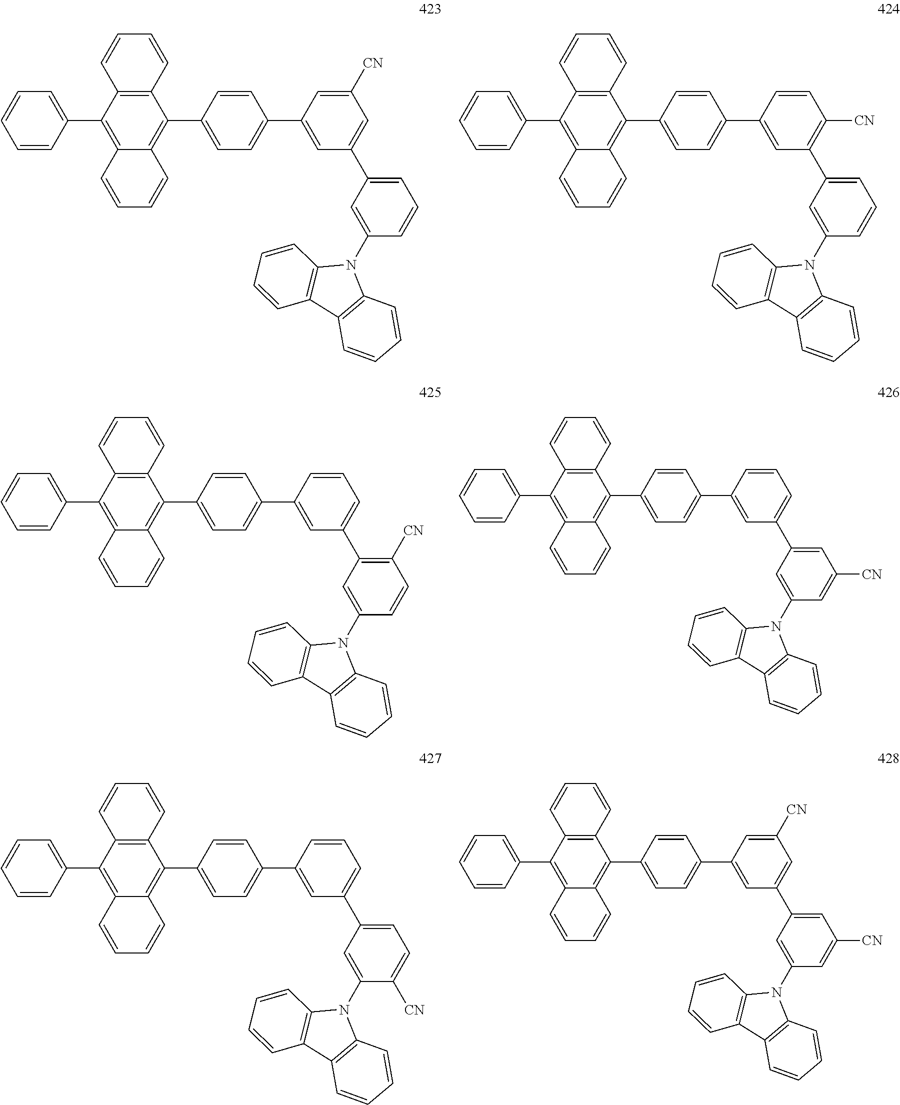

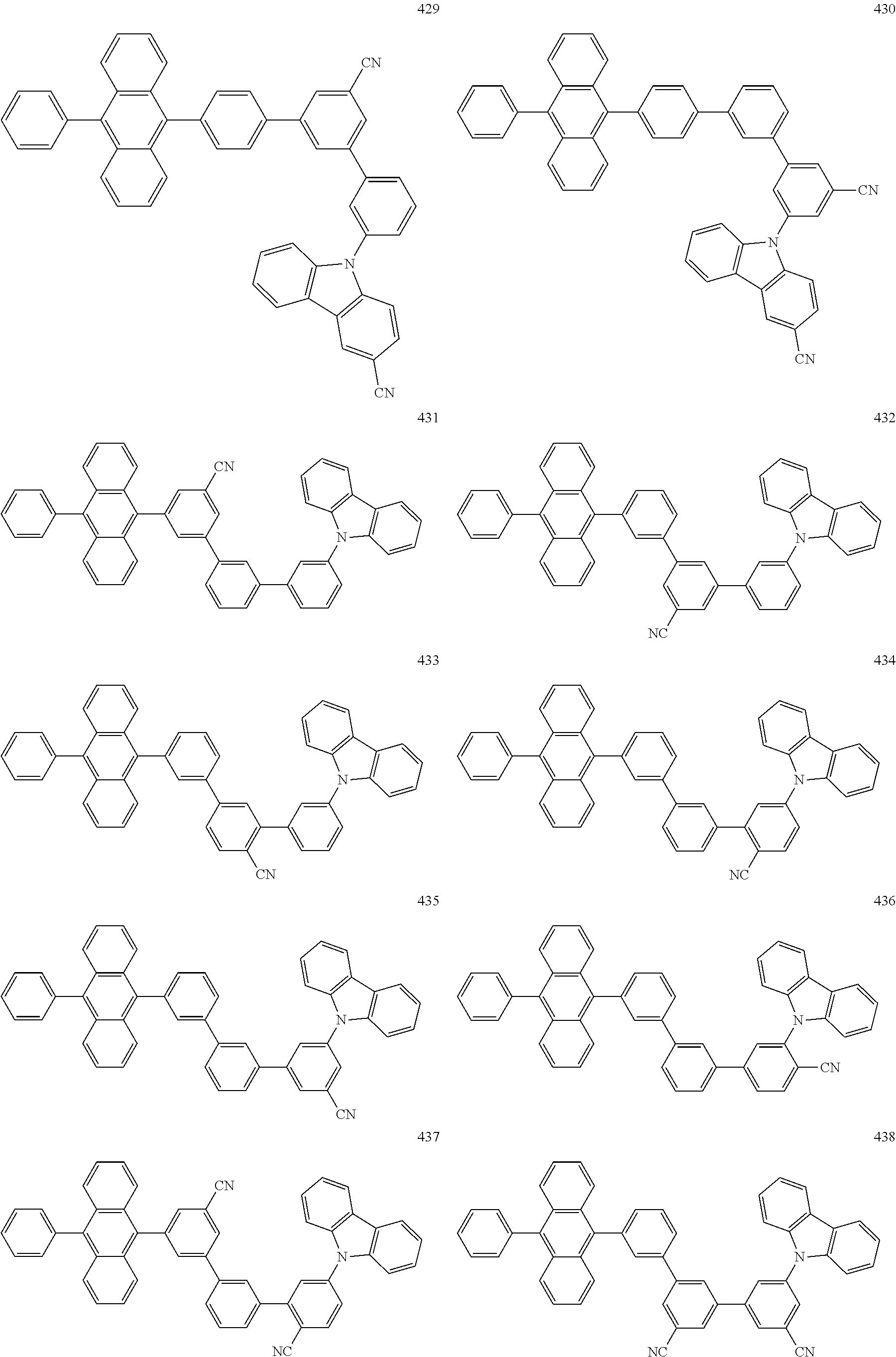

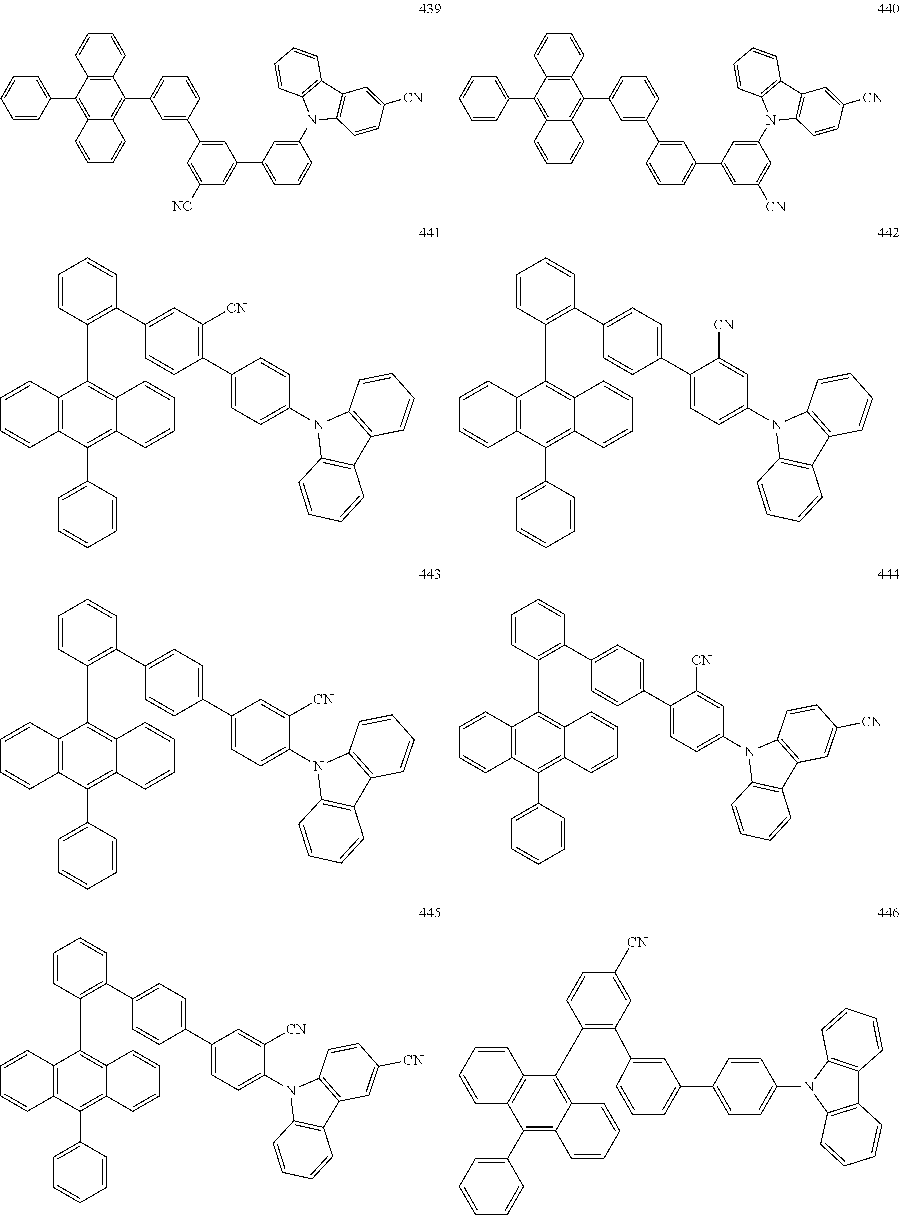

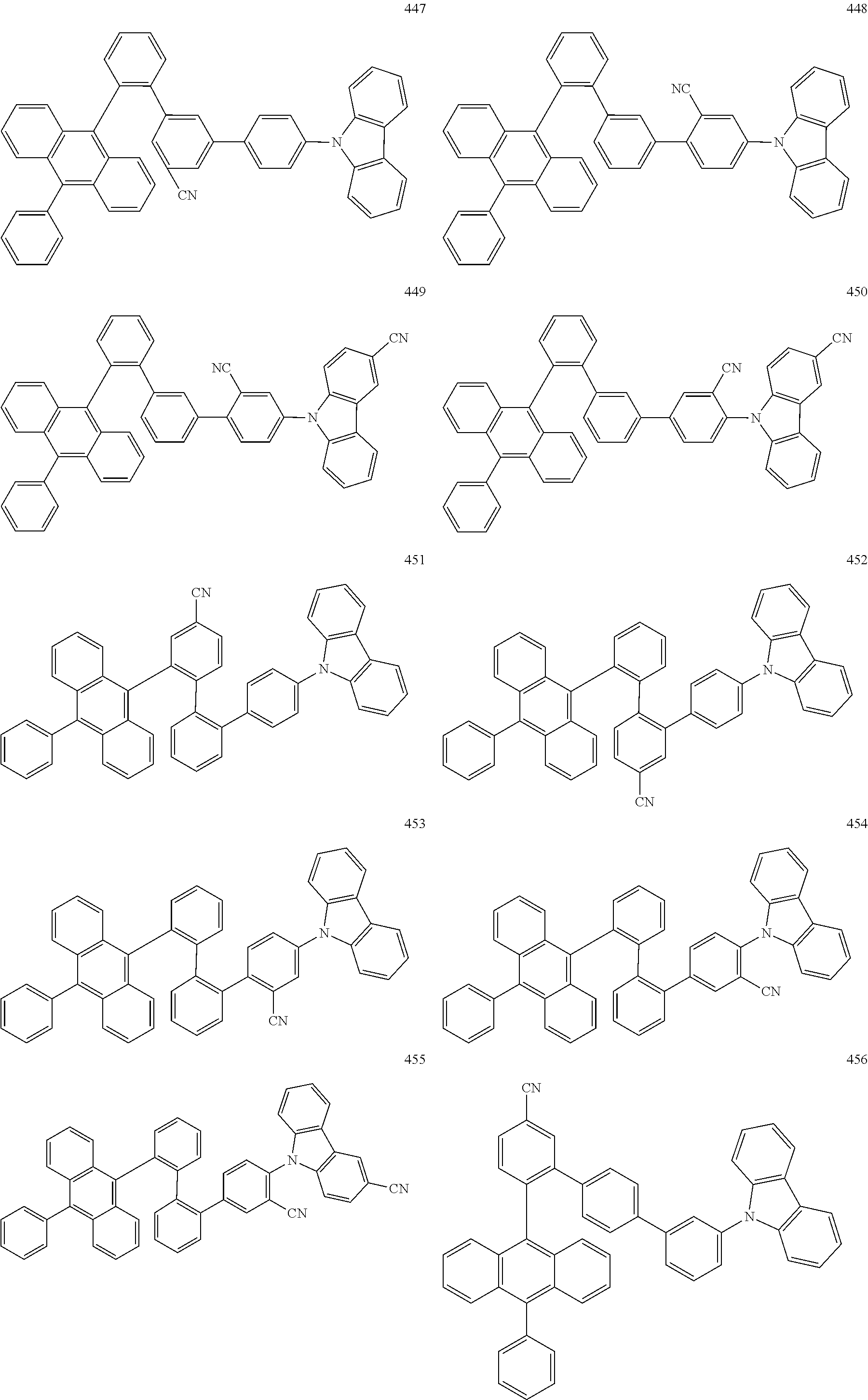

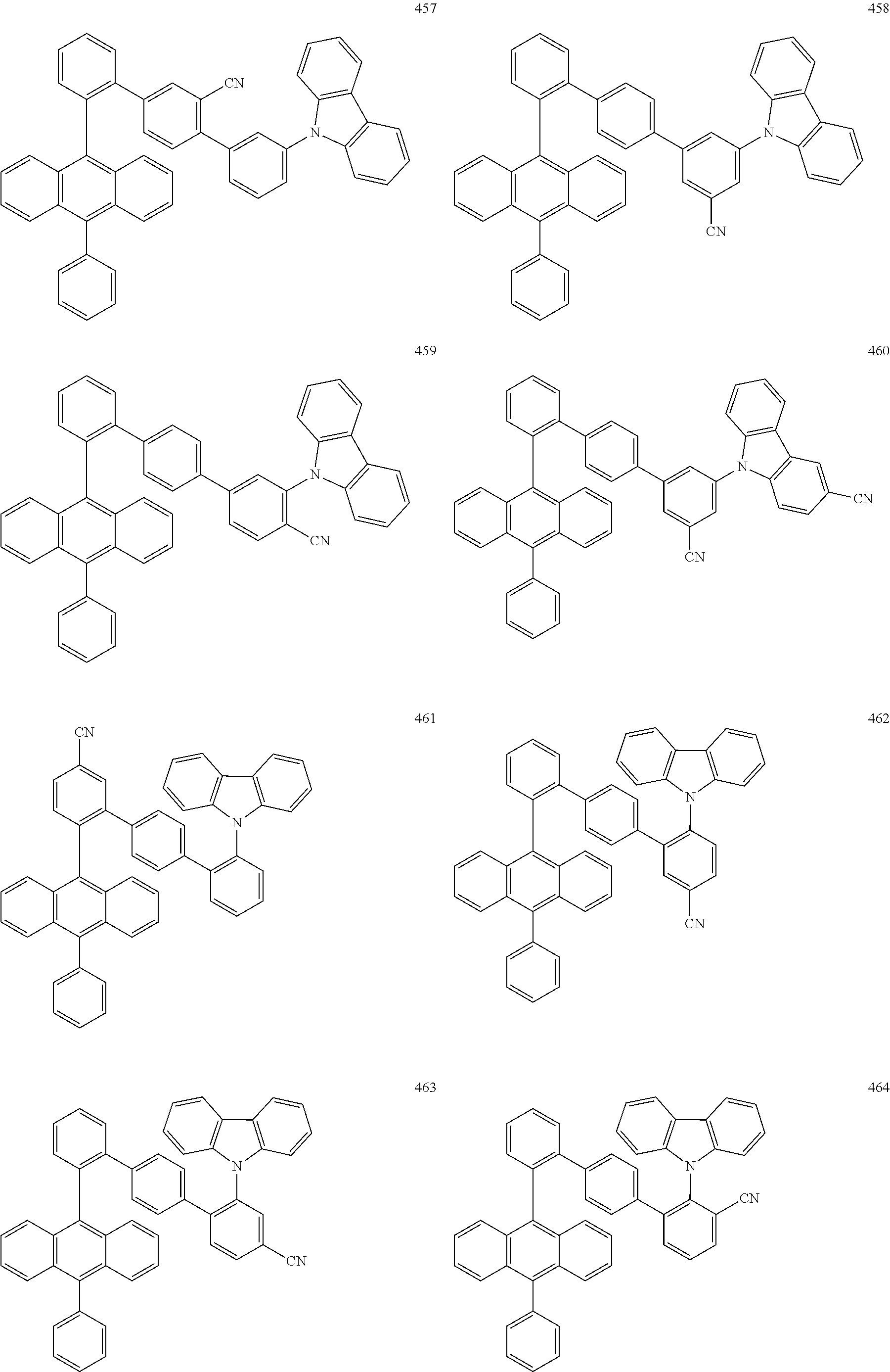

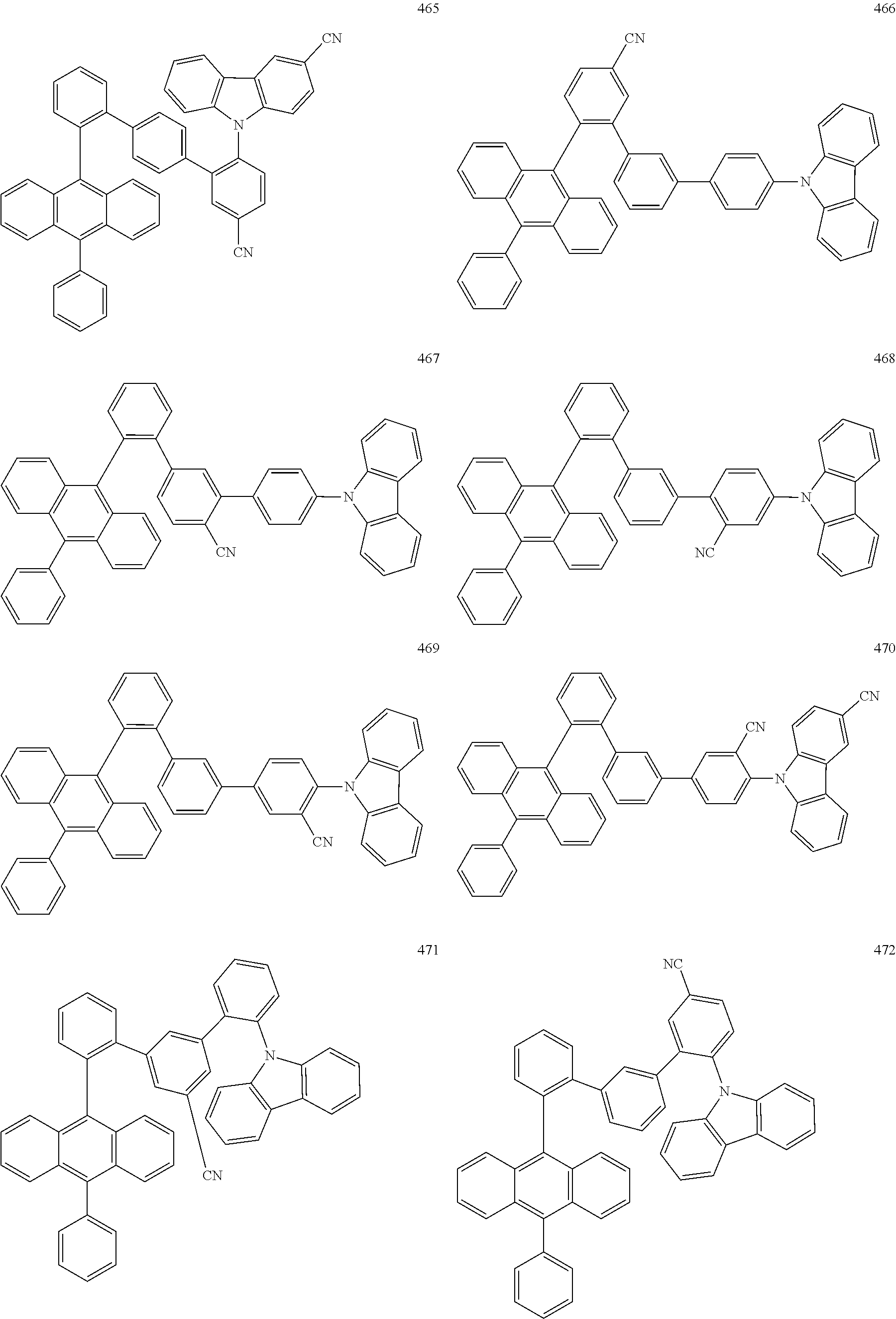

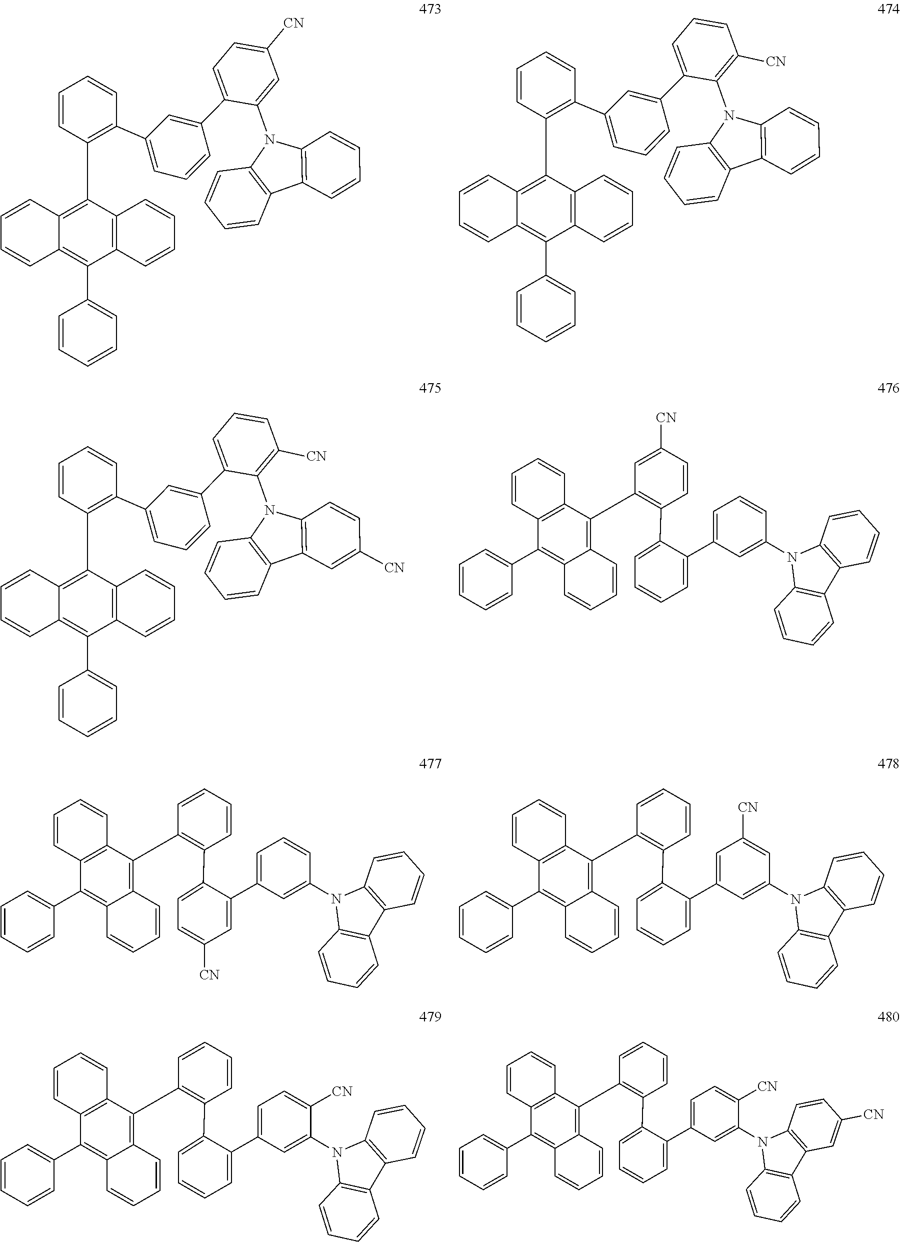

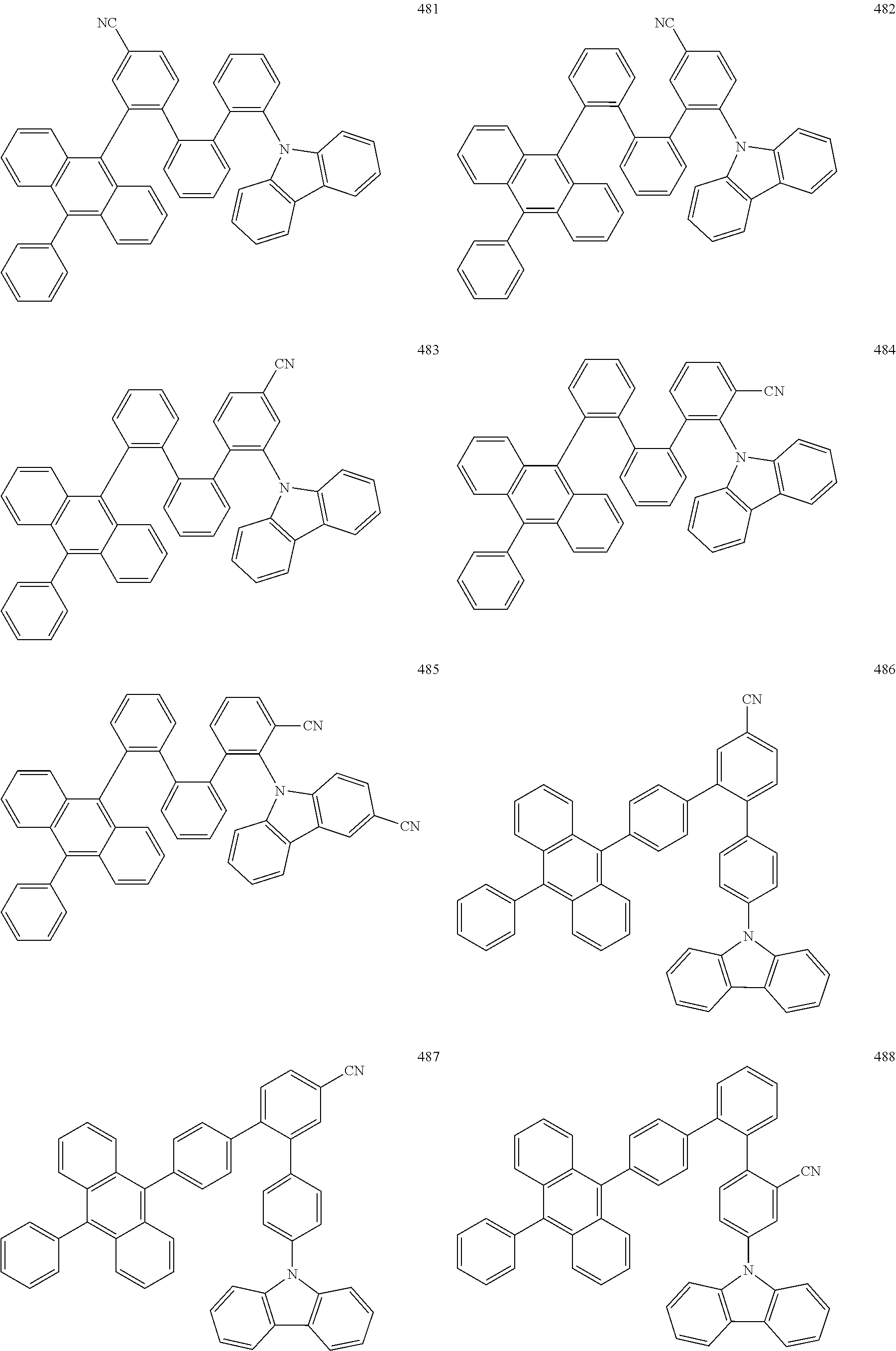

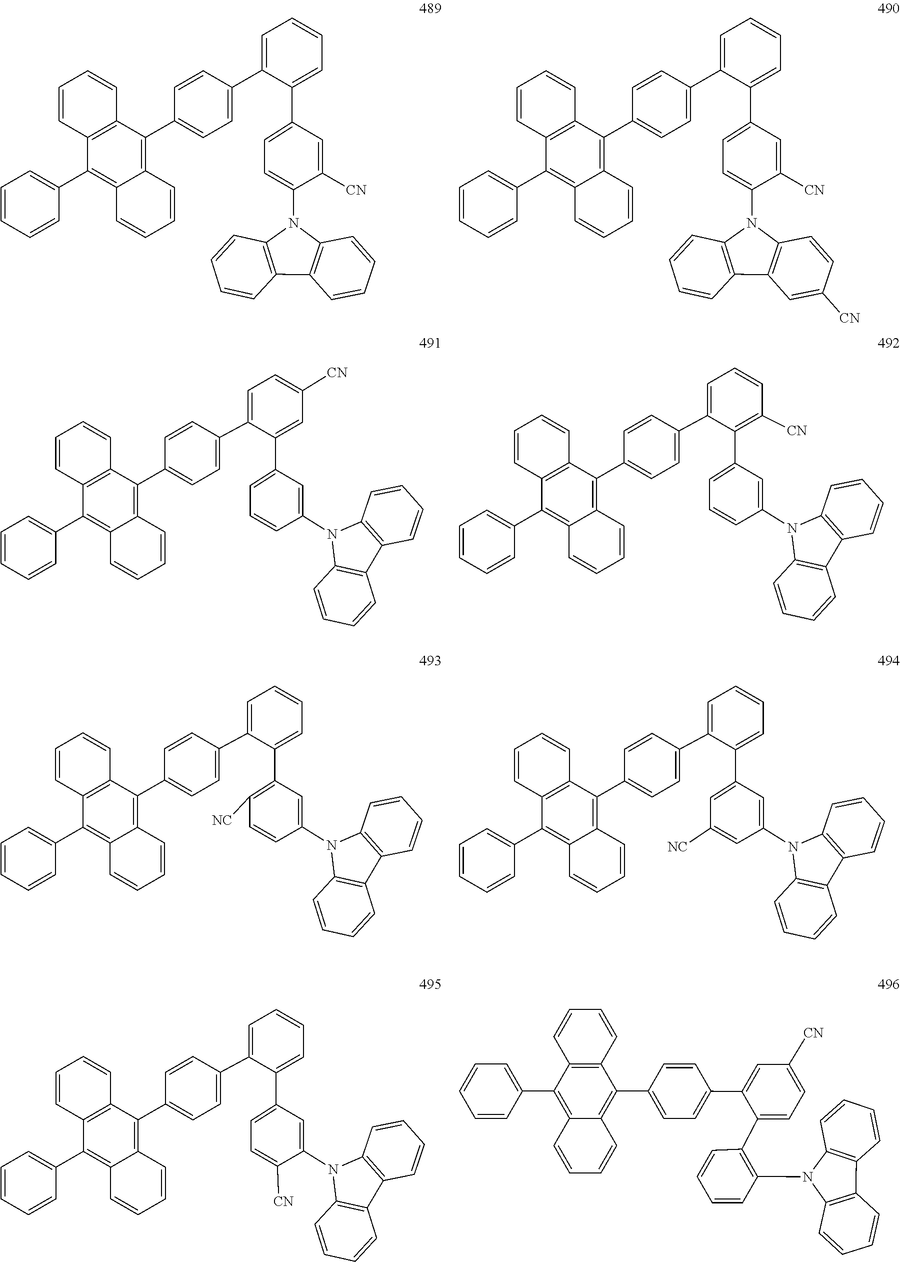

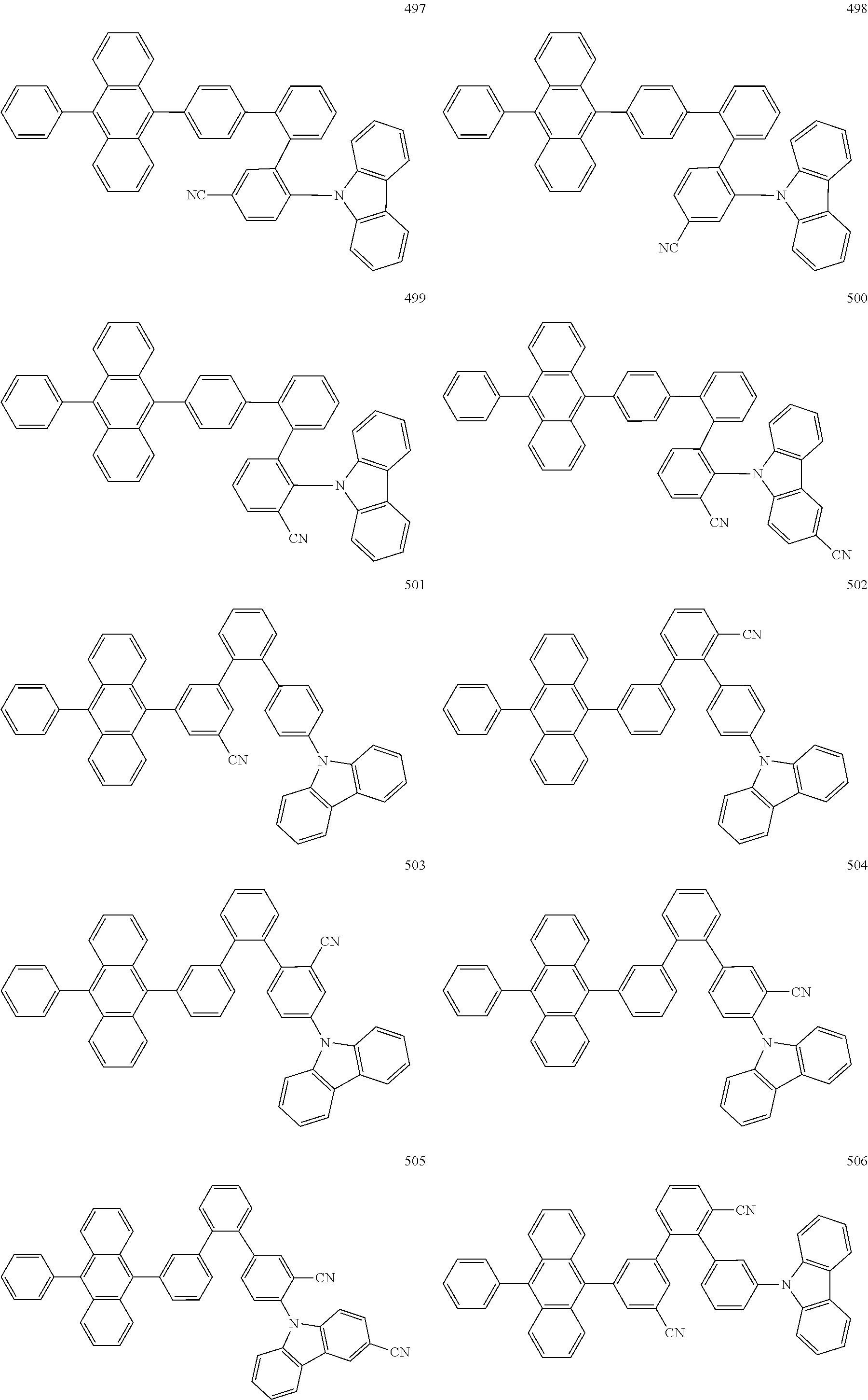

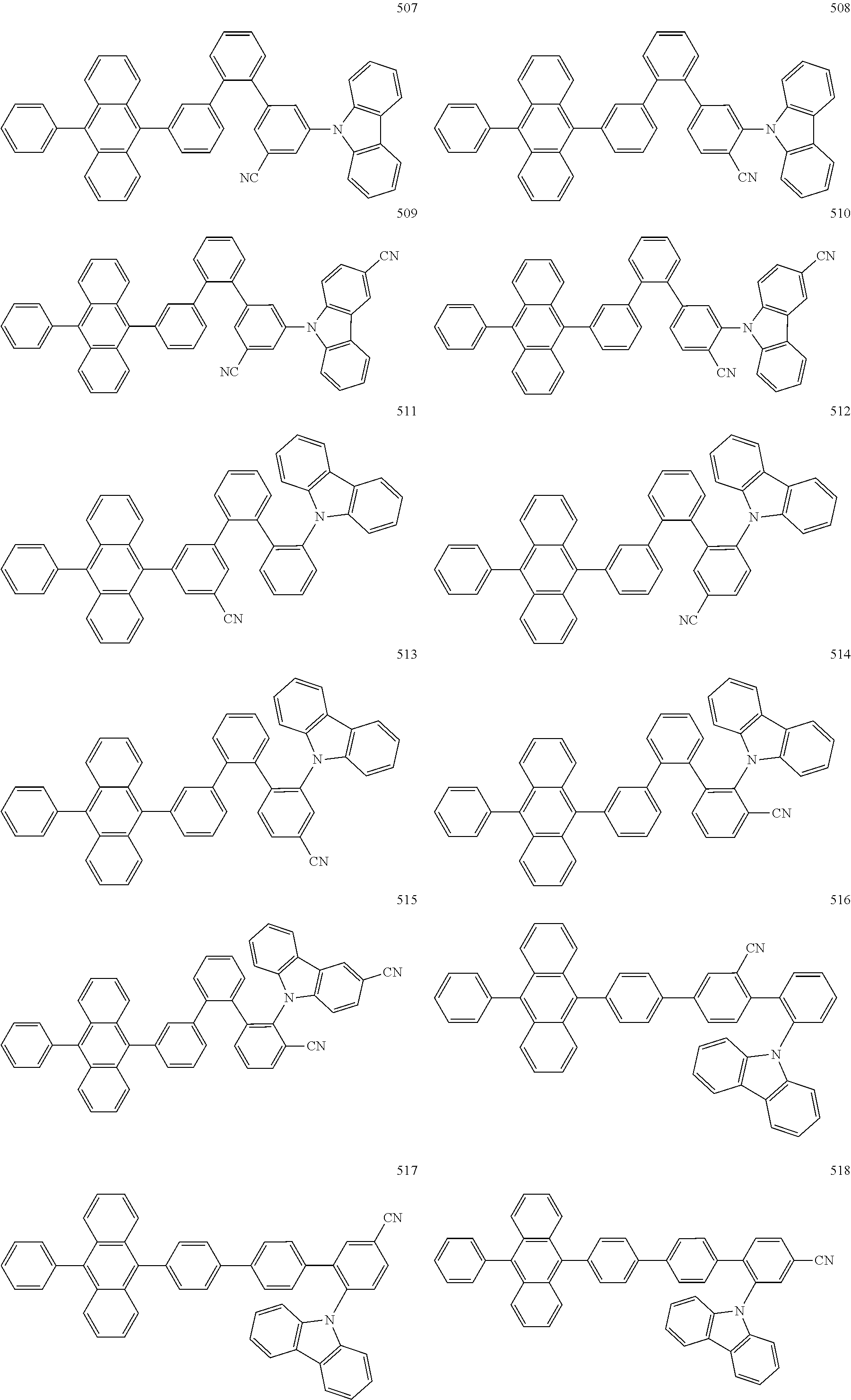

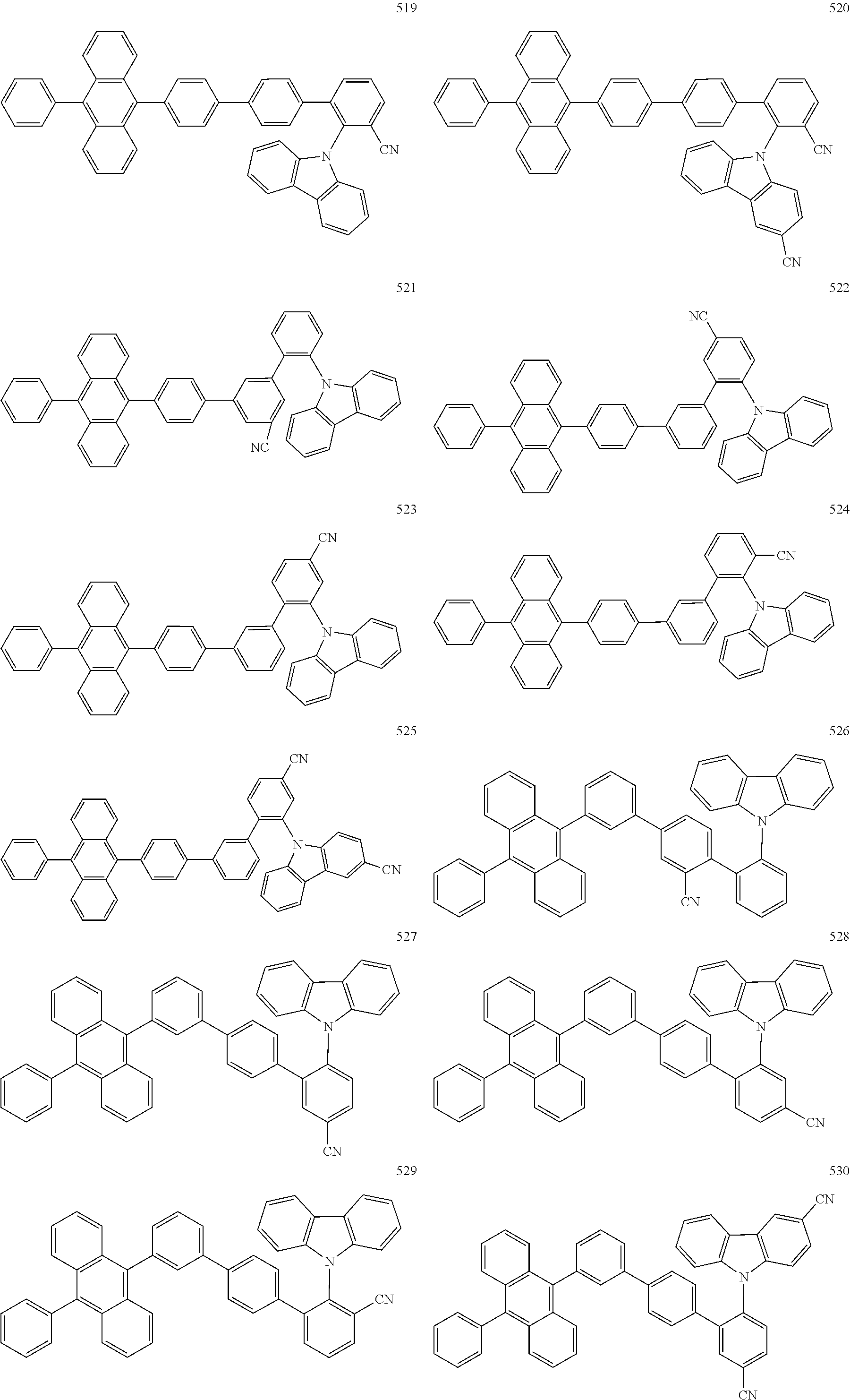

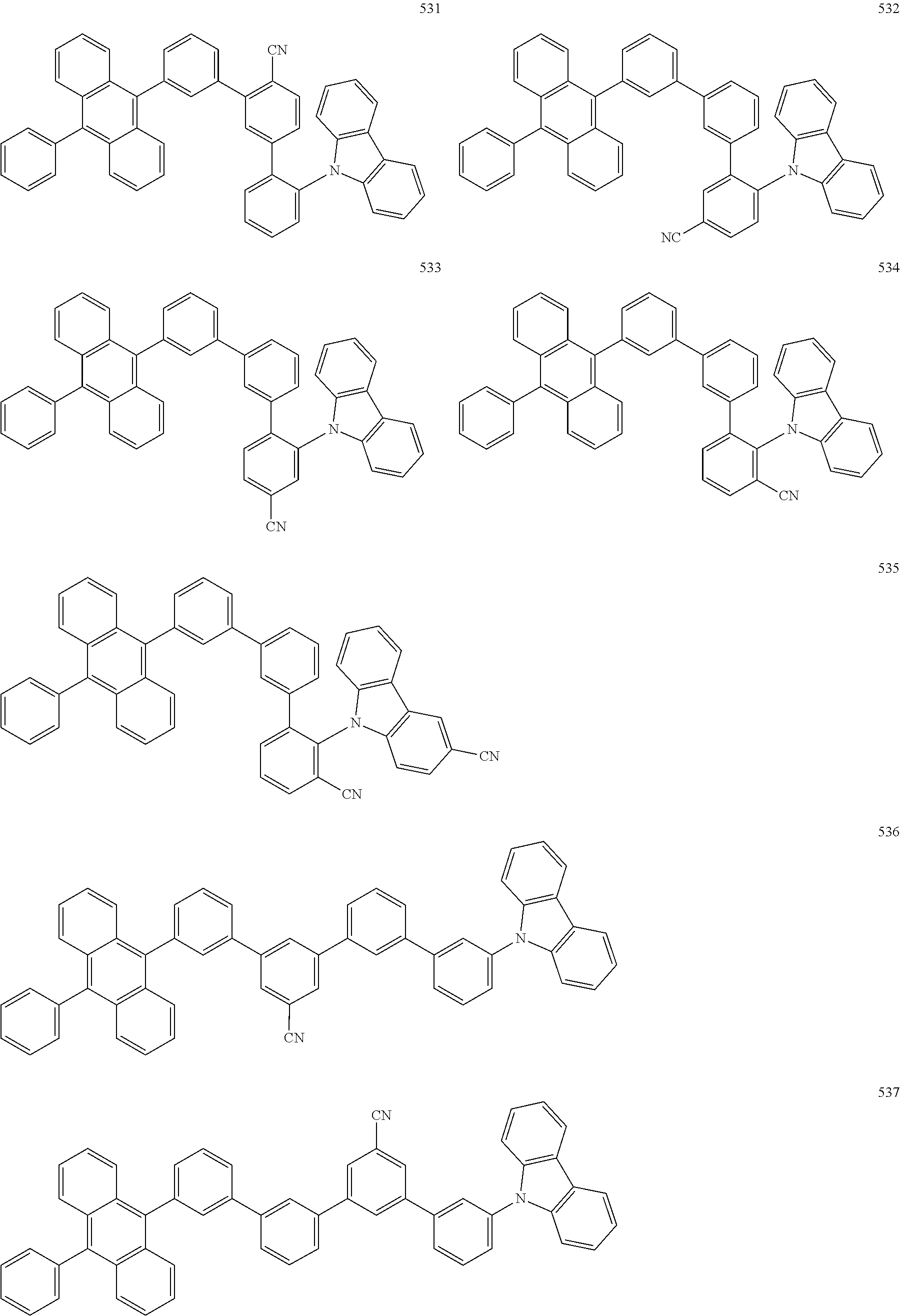

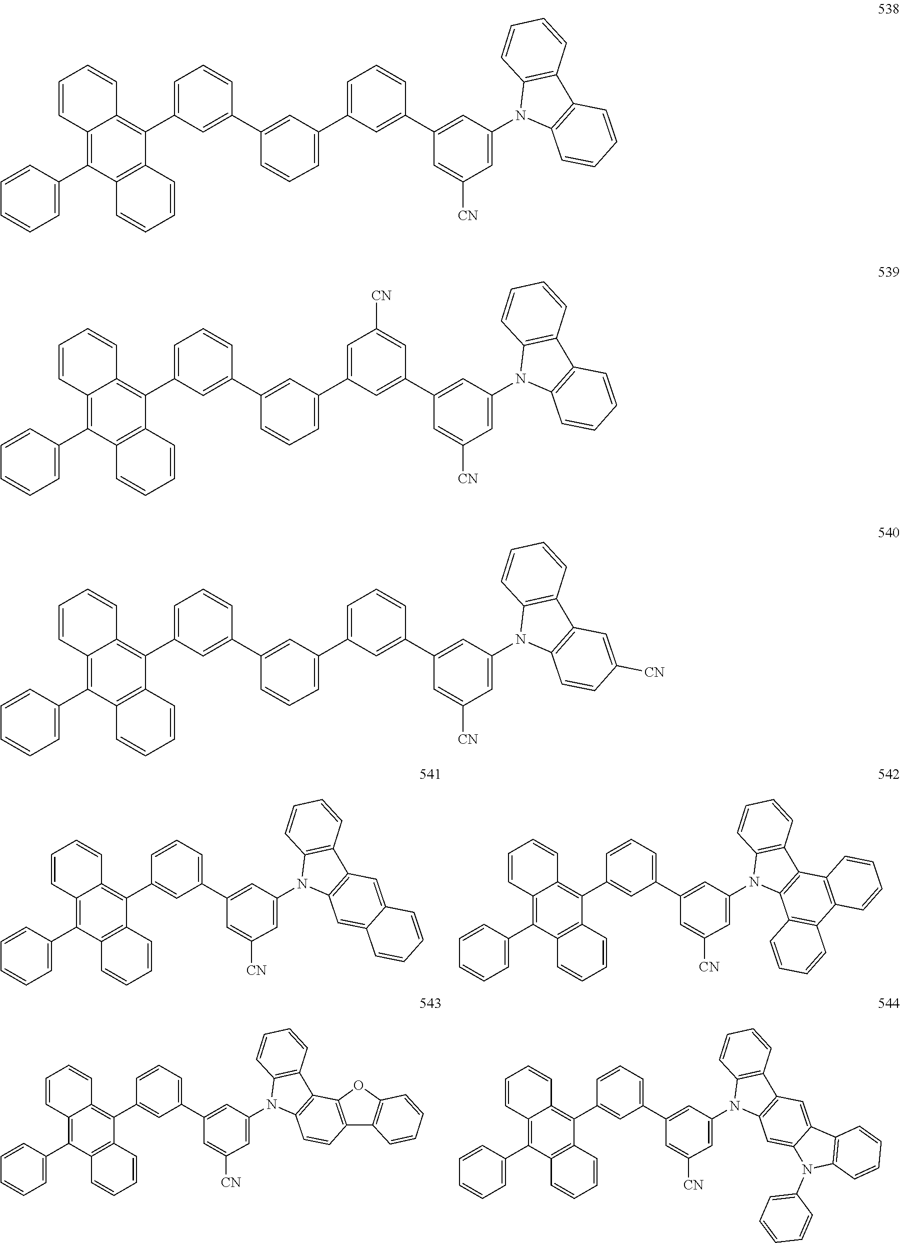

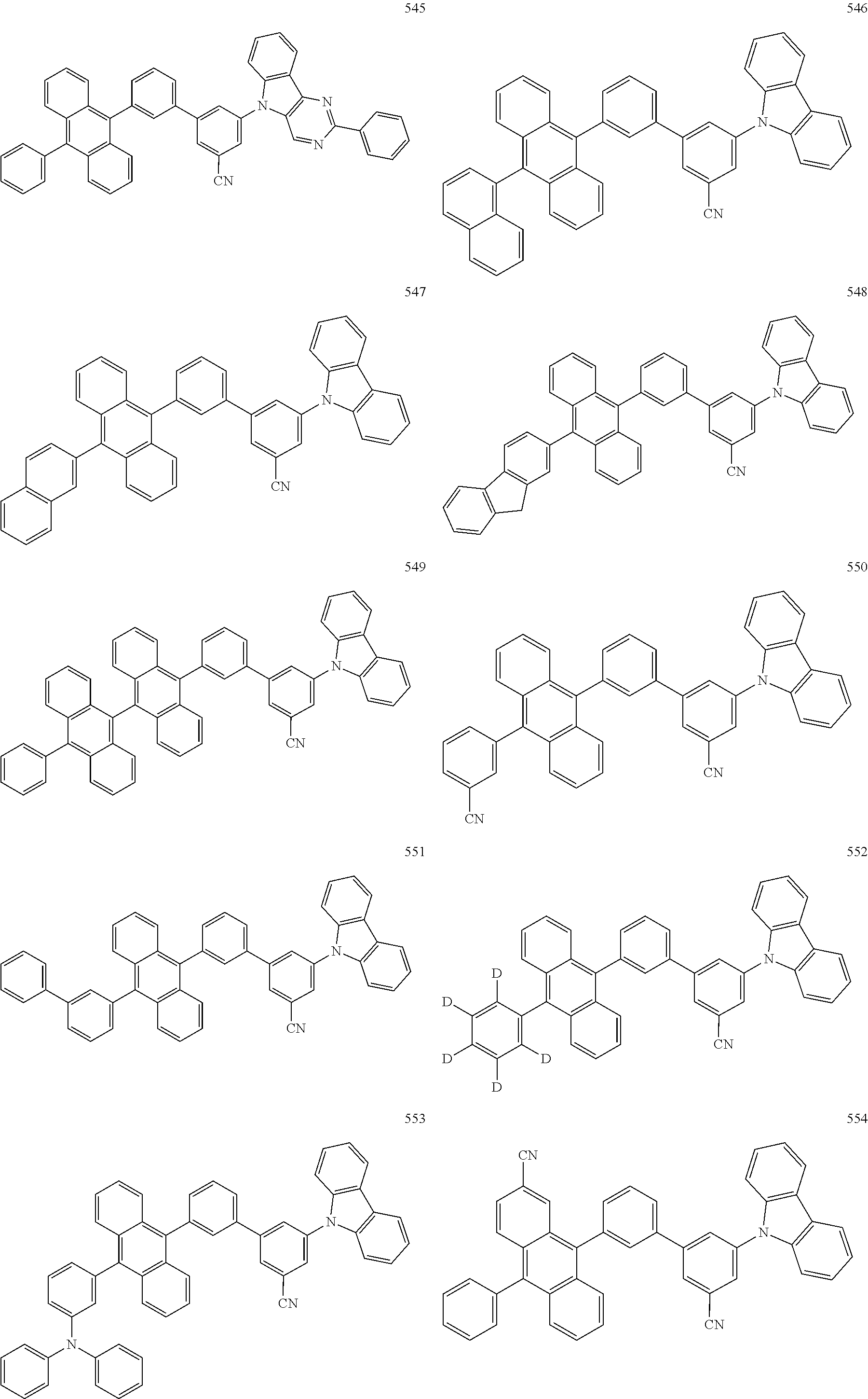

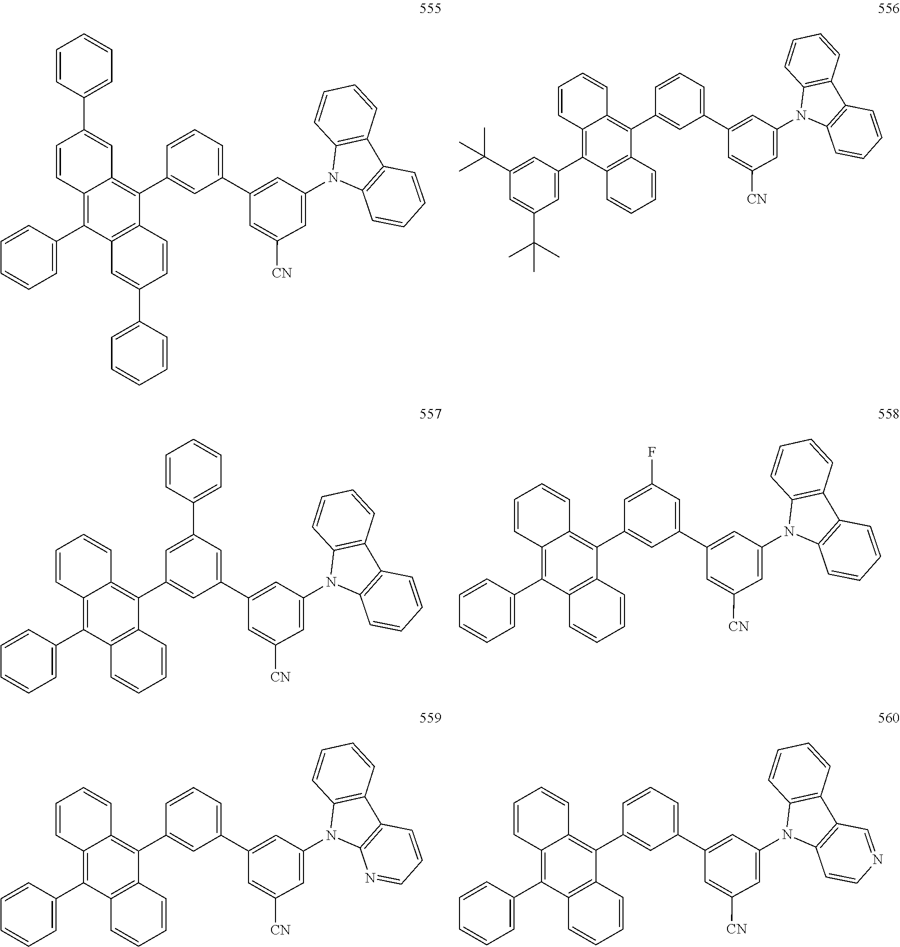

In an embodiment, the fluorescent host may be represented by Formula FH-4:

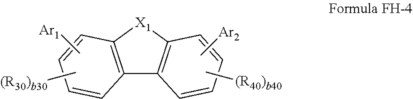

wherein, in Formula FH-4,

X₁ may be O or Se, and

Ar₁ is a group represented by Formula 1A, and Ar₂ is a group represented by Formula 1B,

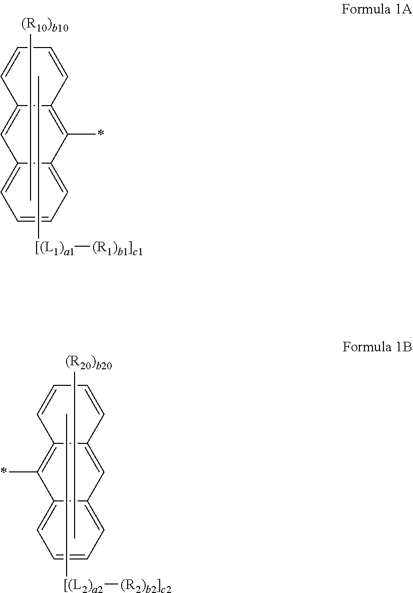

wherein, in the formulae shown above,

L₁ and L₂ may each independently a substituted or unsubstituted C₅-C₃₀ carbocyclic group or a substituted or unsubstituted C₁-C₃₀ heterocyclic group,

a1 and a2 may each independently be an integer from 0 to 3, wherein when a1 is 2 or more, two or more of L₁(s) may be identical to or different from each other, and when a2 is 2 or more, two or more of L₂(s) may be identical to or different from each other,

R₁, R₂, R₁₀, R₂₀, R₃₀, and R₄₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₆₀ heterocycloalkyl group, a substituted or unsubstituted C₃-C₁₀ cycloalkenyl group, a substituted or unsubstituted C₁-C₆₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

b1 and b2 may each independently be an integer from 1 to 5,

when b1 is 2 or more, two or more of R₁(s) may be identical to or different from each other, and when b2 is 2 or more, two or more of R₂ may be identical to or different from each other,

b10 and b20 may each independently an integer from 1 to 8,

b30 and b40 may each independently an integer from 1 to 3,

c1 and c2 may each independently be an integer from 1 to 8, and

the sum of b10 and c1 may be 9 and the sum of b20 and c2 may be 9.

In an embodiment, the fluorescent host represented by Formula FH-4 may be Group FH4, but embodiments of the present disclosure are not limited thereto:

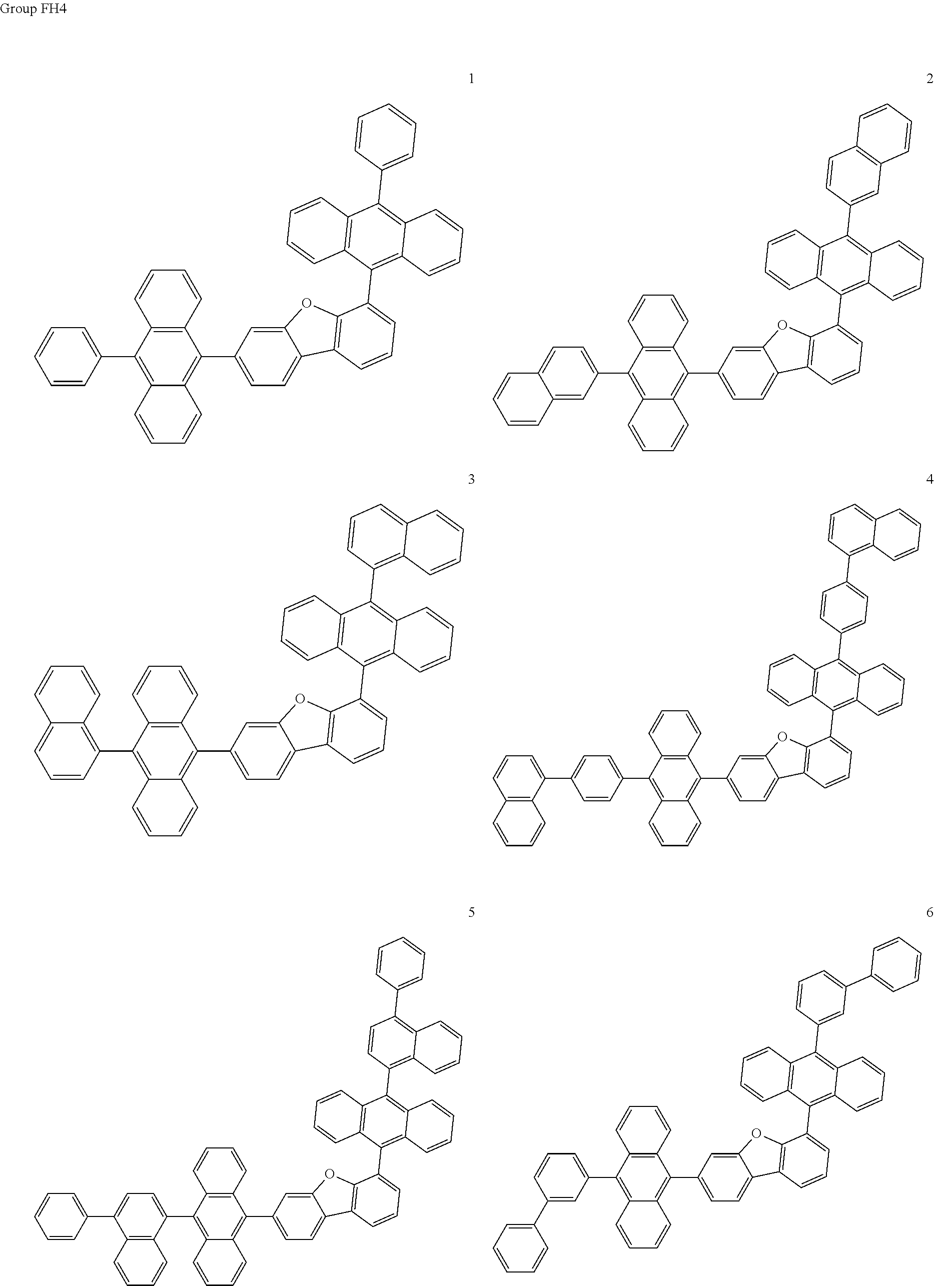

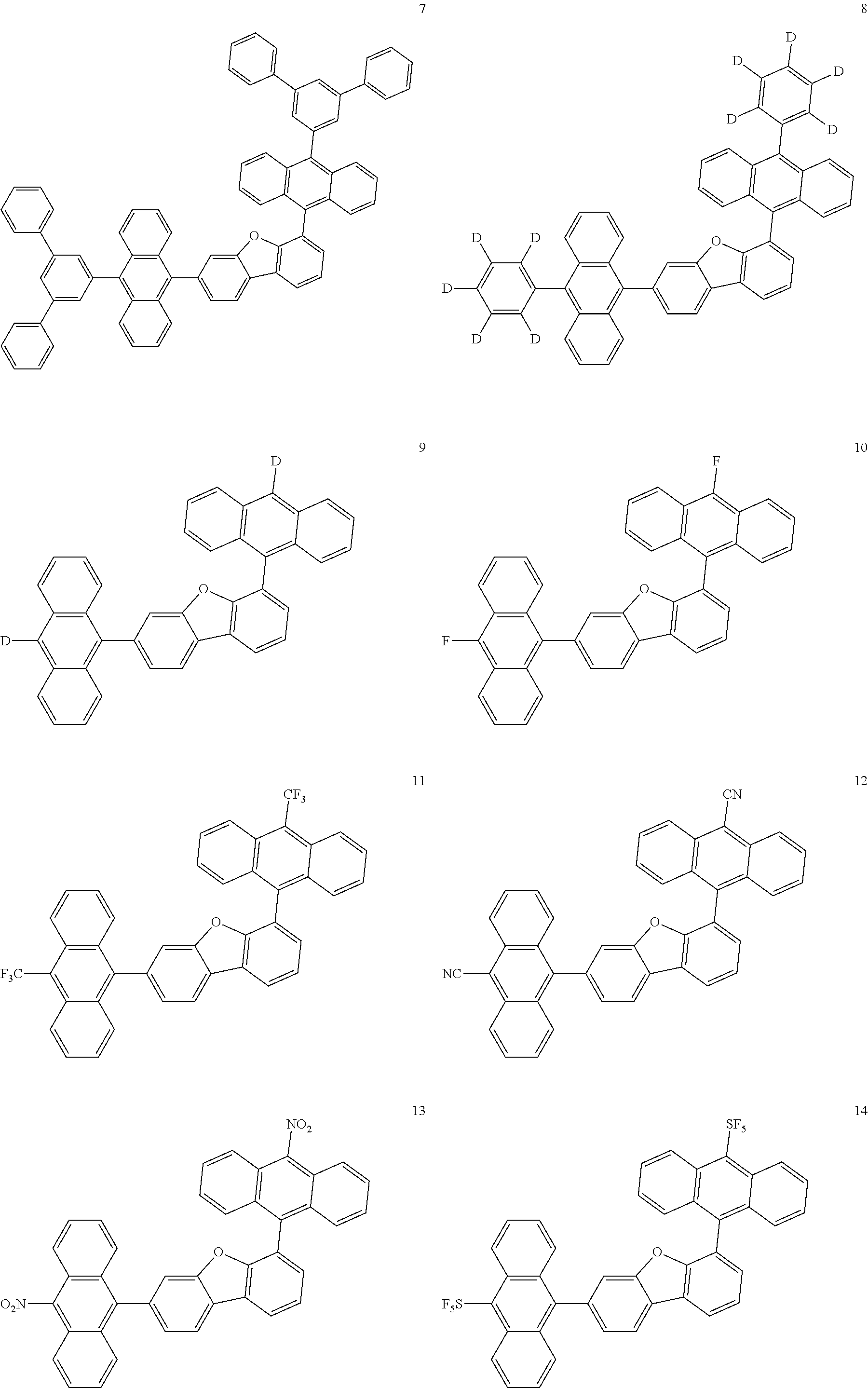

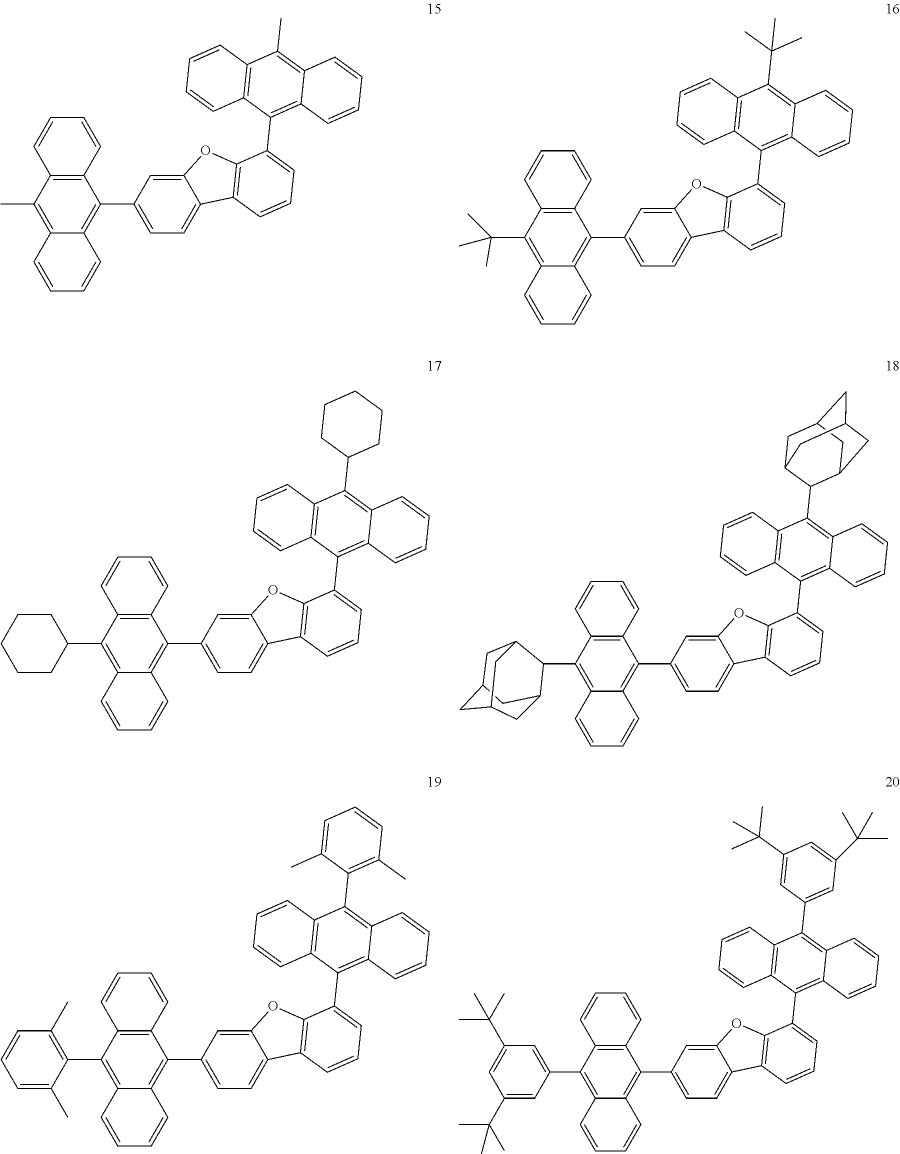

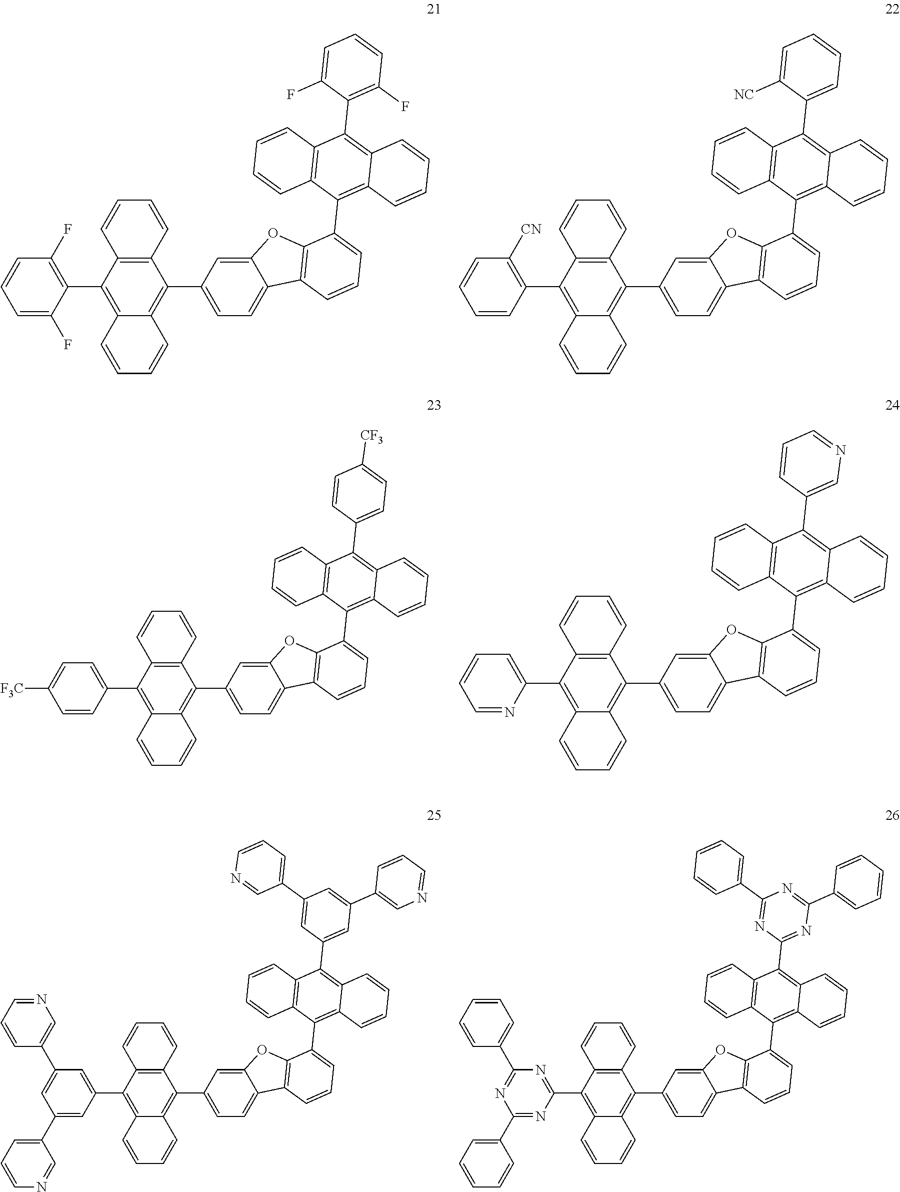

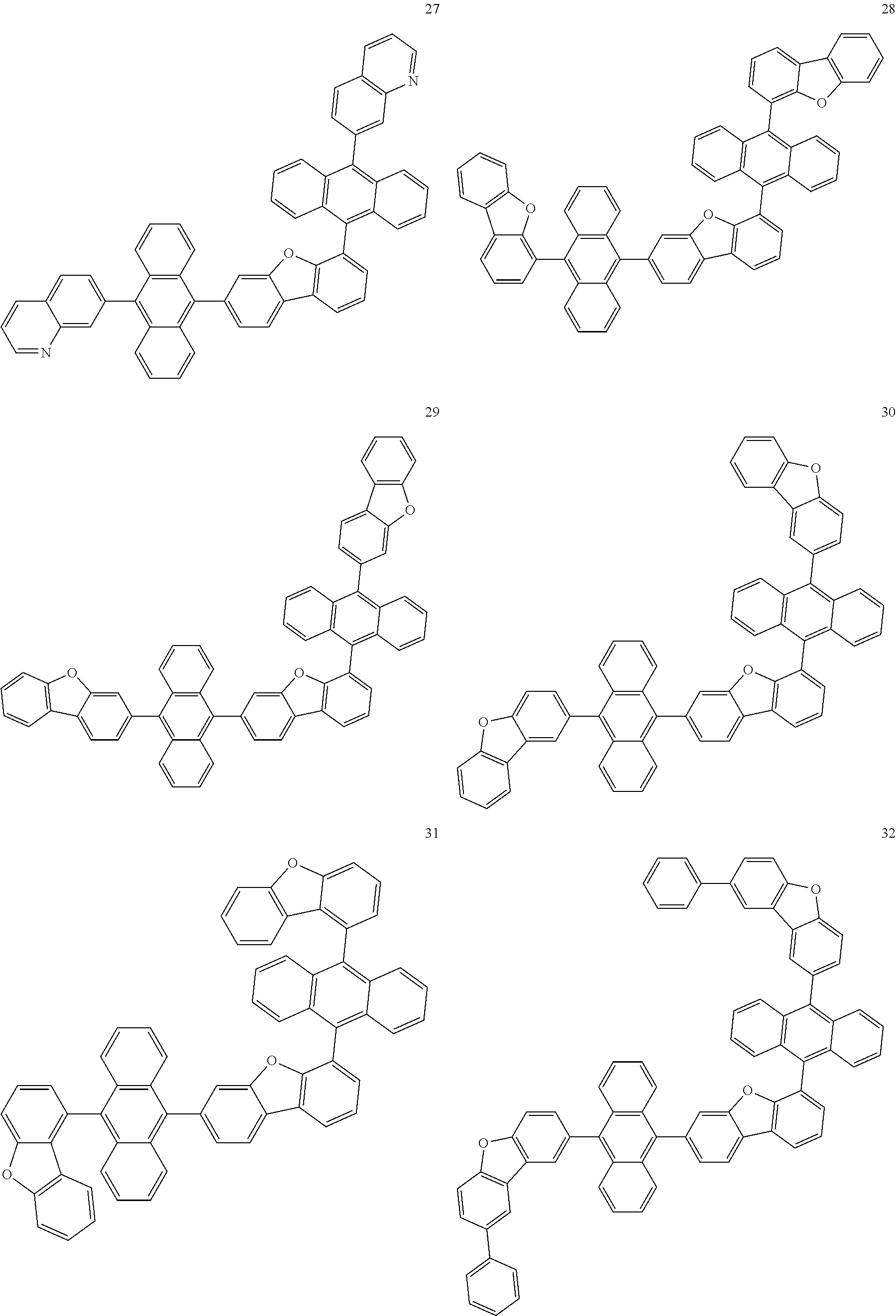

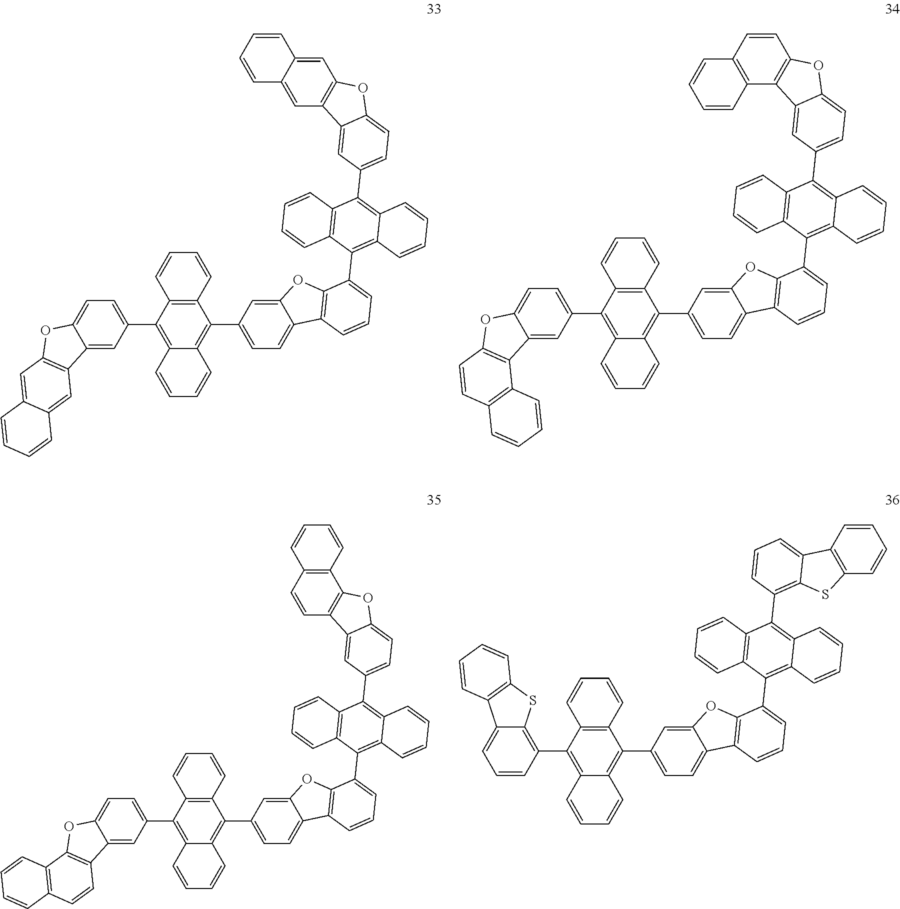

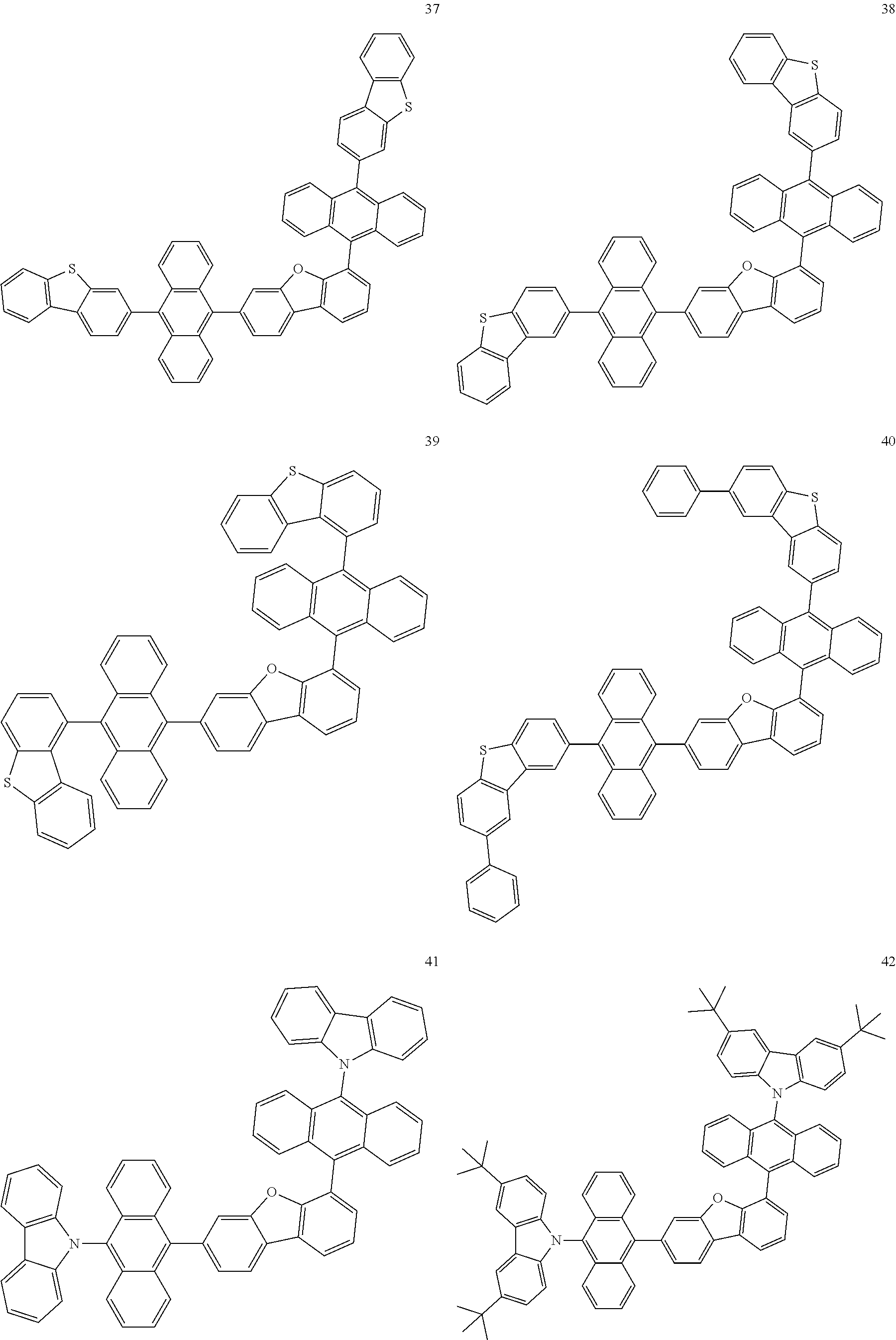

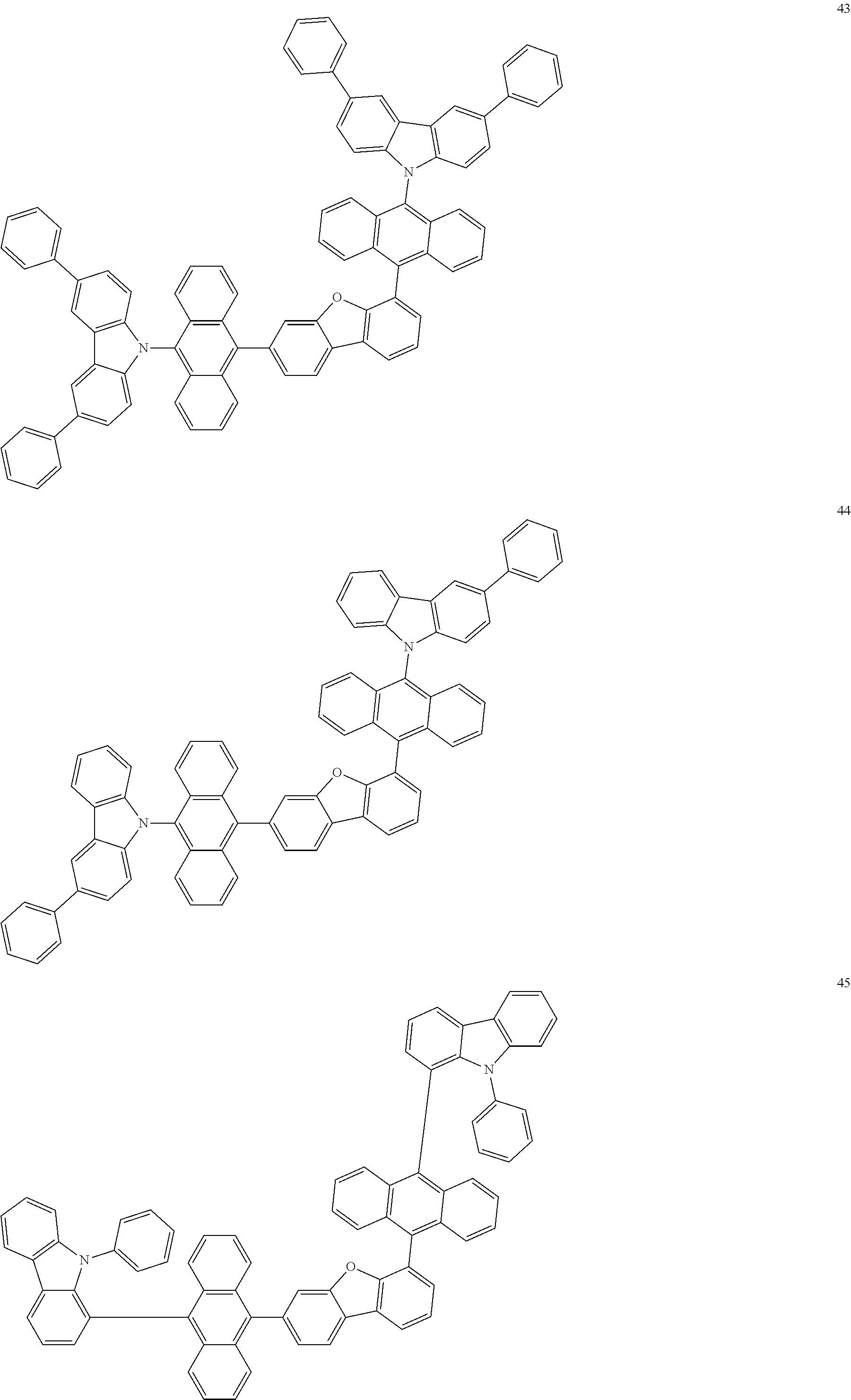 [SMILES: c1ccc(-c2ccc3c(c2)c2cc(-c4ccccc4)ccc2n3-c2c3ccccc3c(-c3ccc4c(c3)oc3c(-c5c6ccccc6c(-n6c7ccc(-c8ccccc8)cc7c7cc(-c8ccccc8)ccc76)c6ccccc56)cccc34)c3ccccc23)cc1 ; c1ccc(-c2ccc3c(c2)c2ccccc2n3-c2c3ccccc3c(-c3ccc4c(c3)oc3c(-c5c6ccccc6c(-n6c7ccccc7c7cc(-c8ccccc8)ccc76)c6ccccc56)cccc34)c3ccccc23)cc1 ; c1ccc(-n2c3ccccc3c3cccc(-c4c5ccccc5c(-c5ccc6c(c5)oc5c(-c7c8ccccc8c(-c8cccc9c%10ccccc%10n(-c%10ccccc%10)c89)c8ccccc78)cccc56)c5ccccc45)c32)cc1]

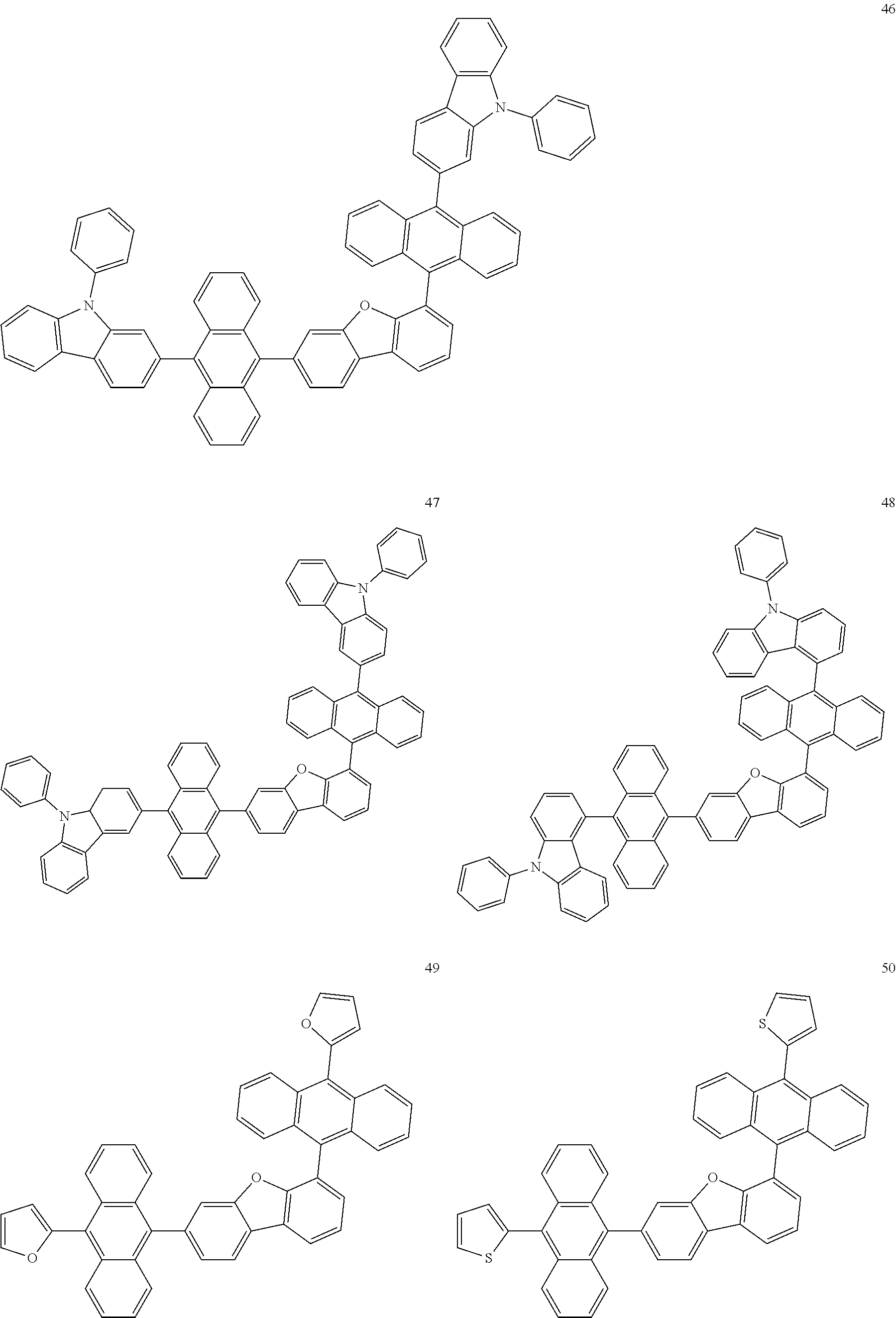

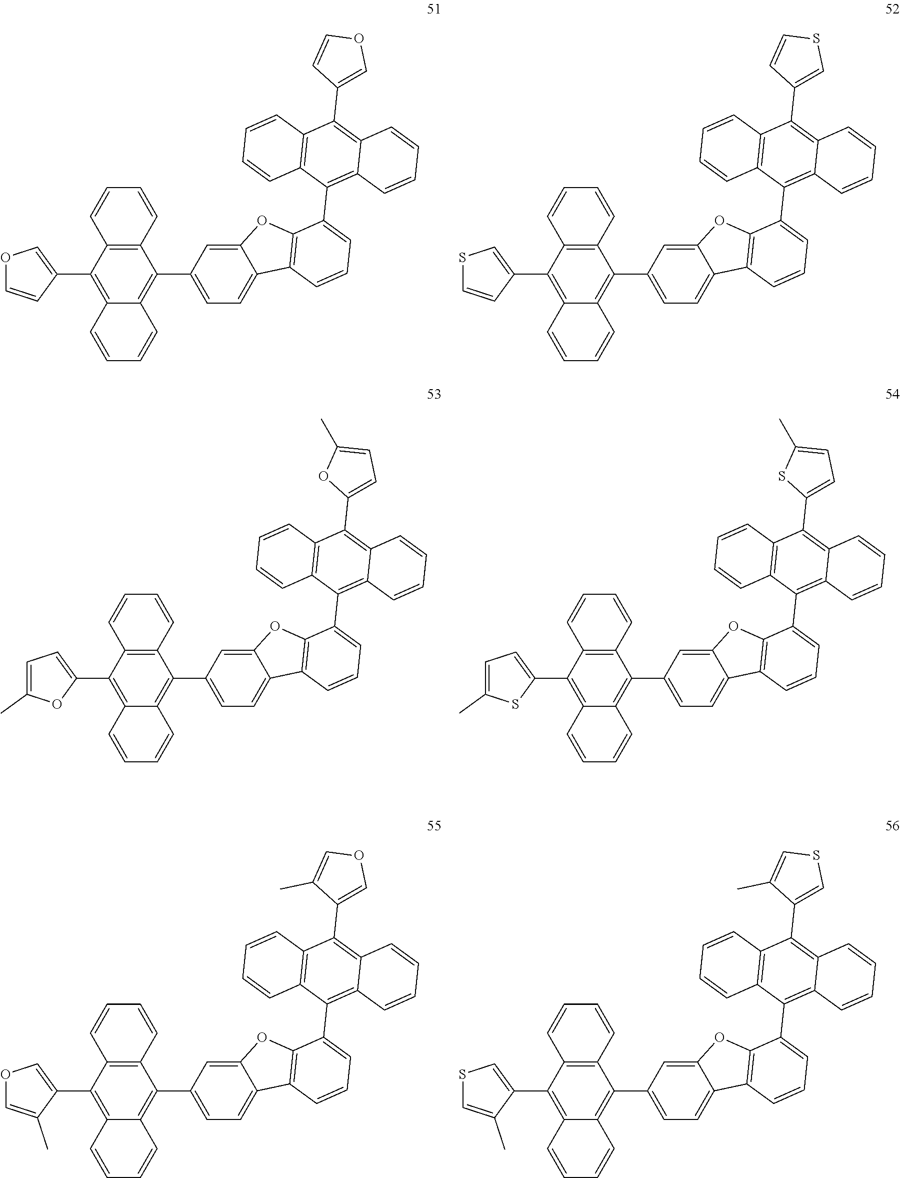

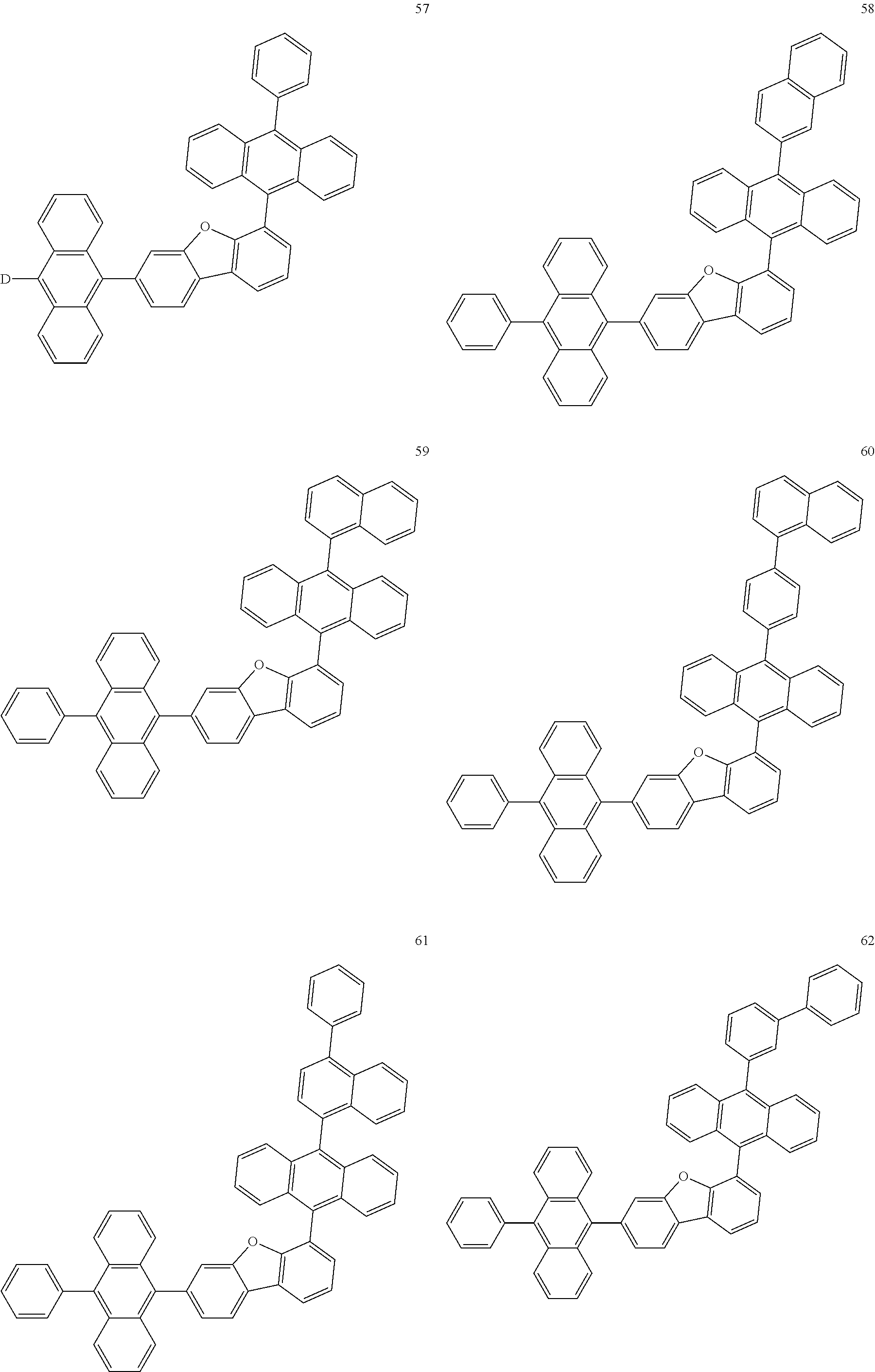

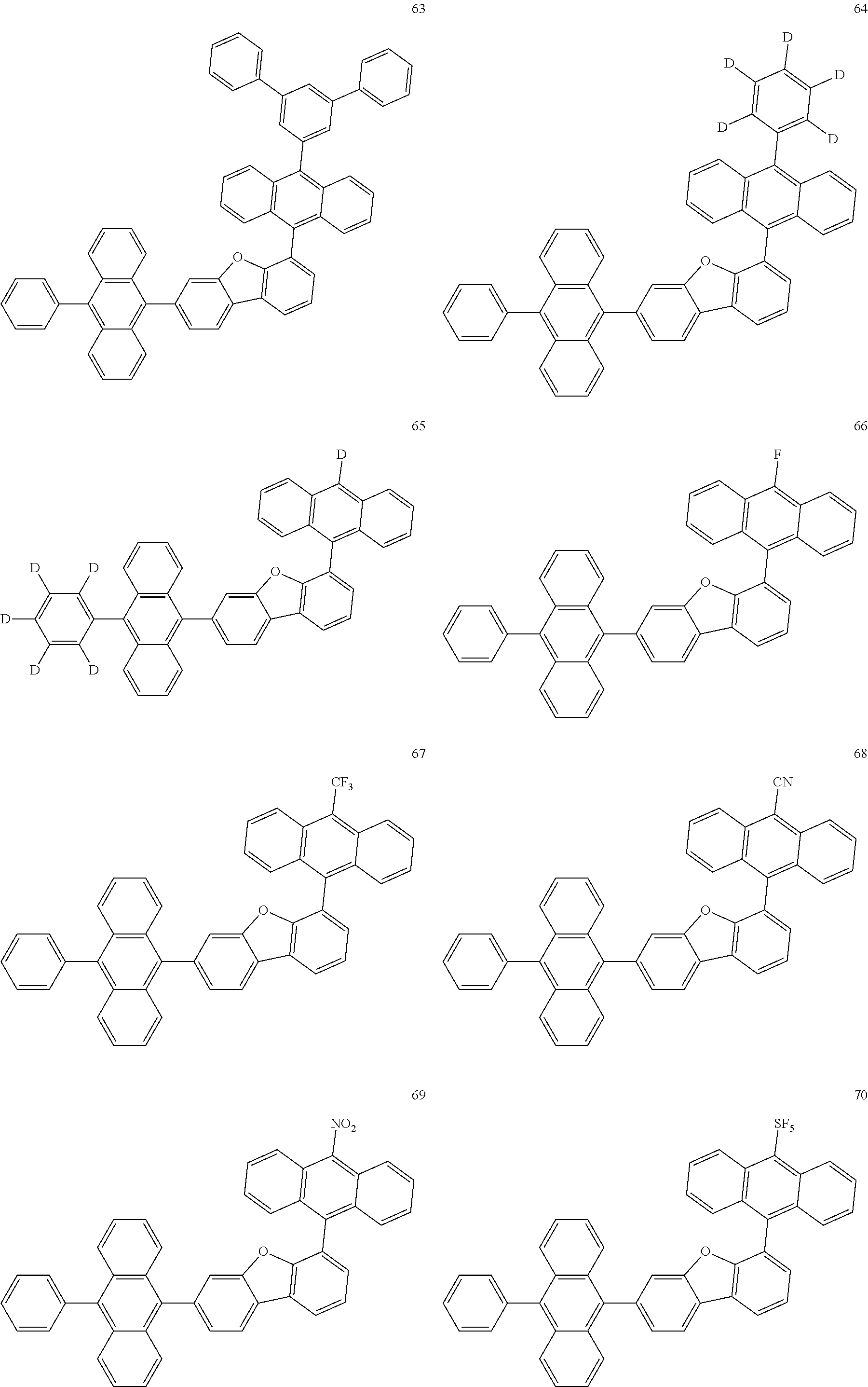

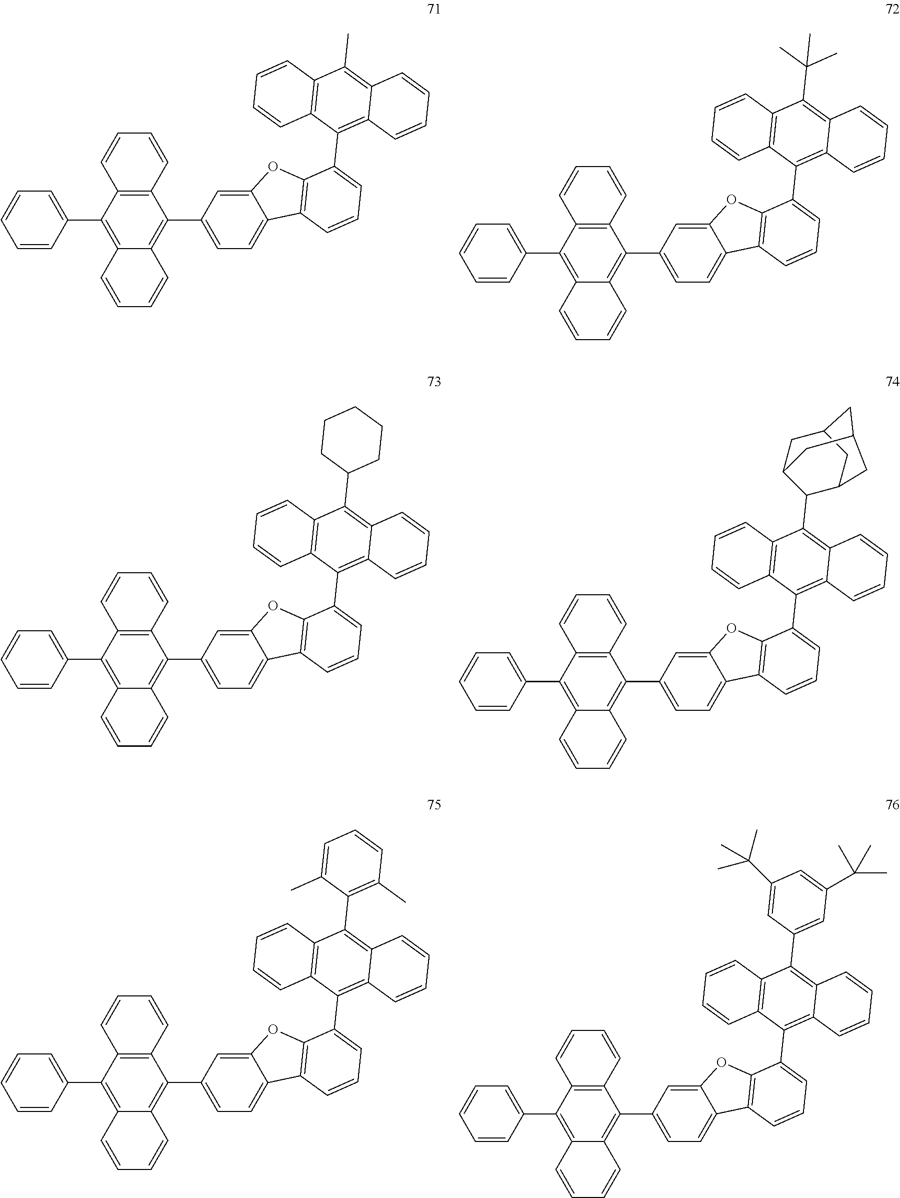

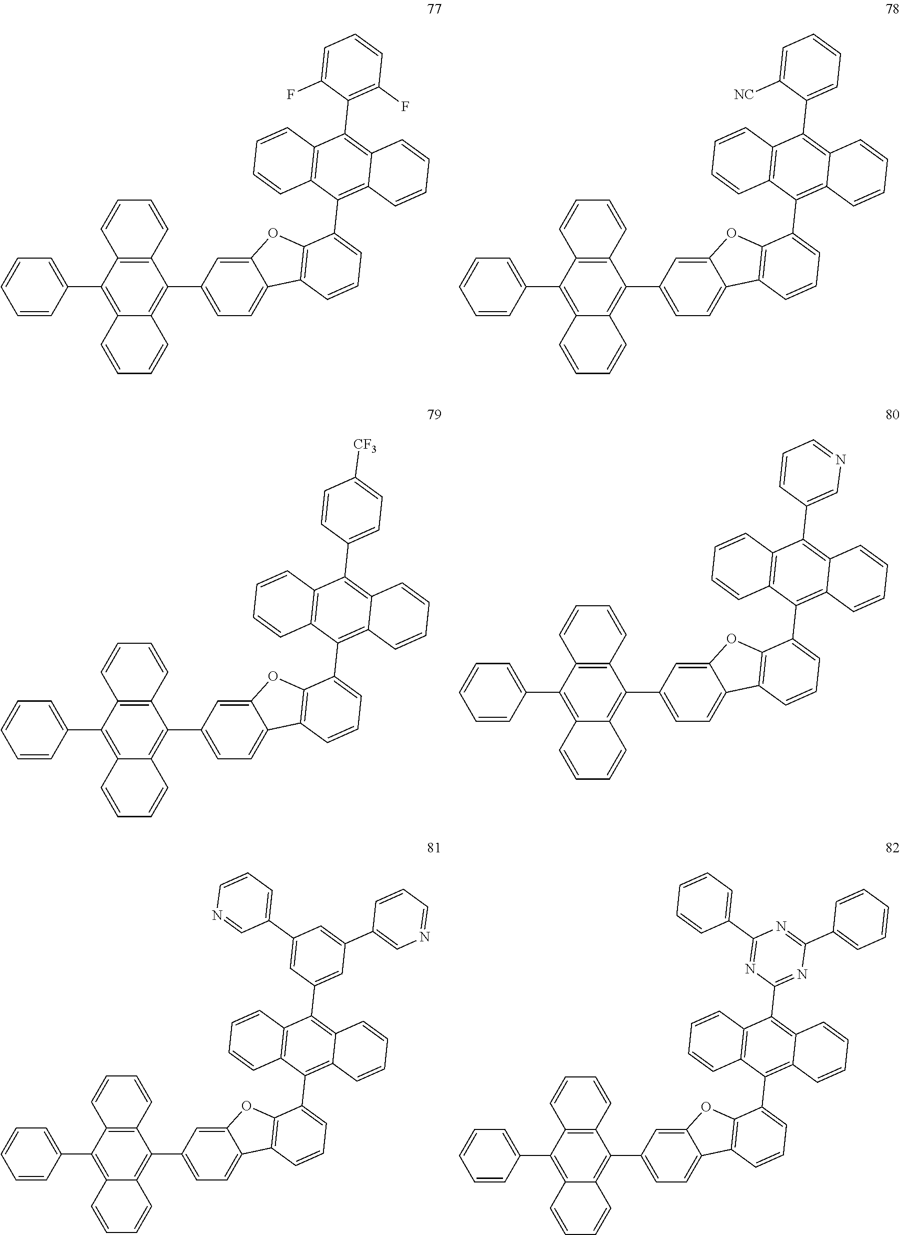

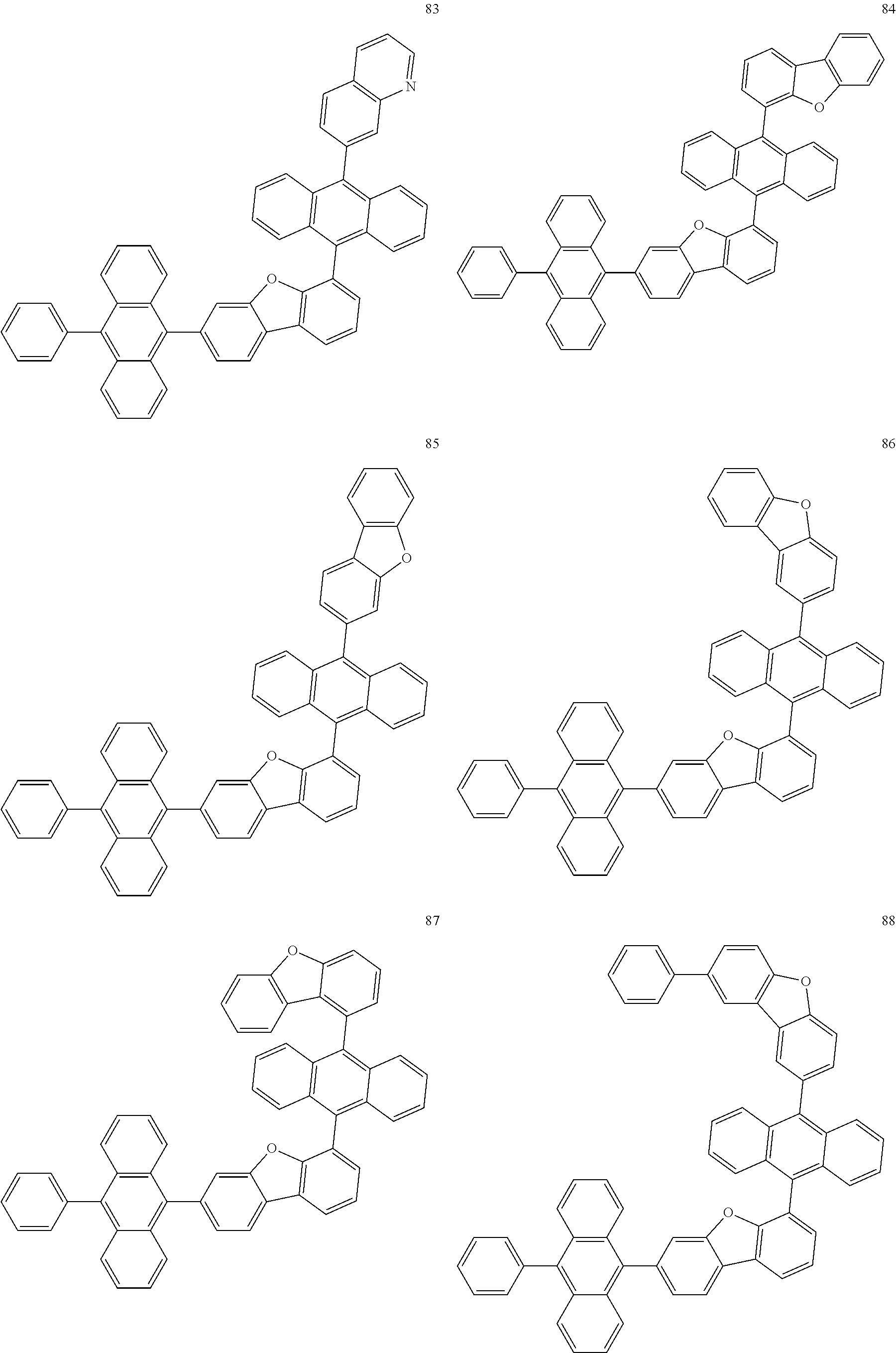

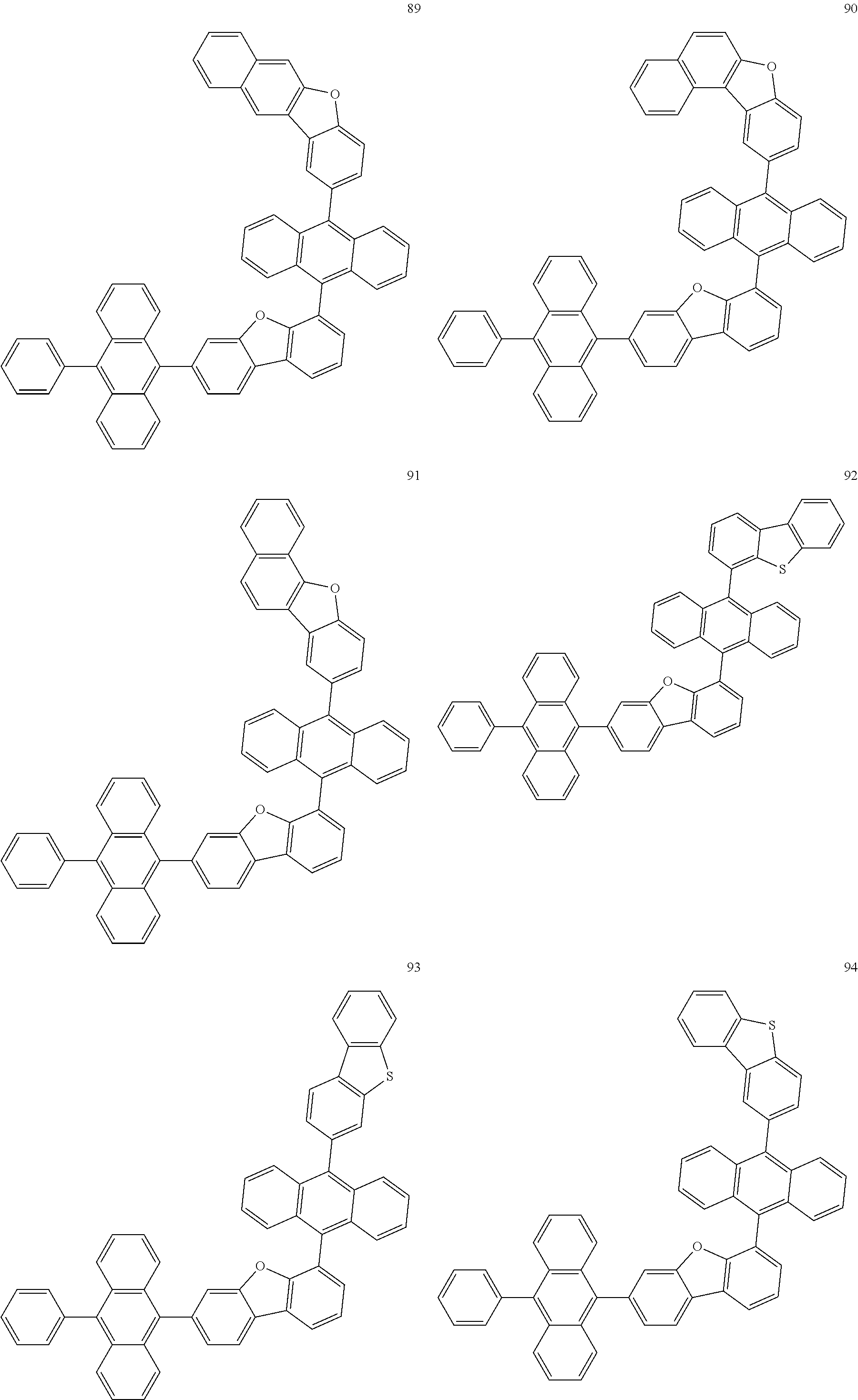

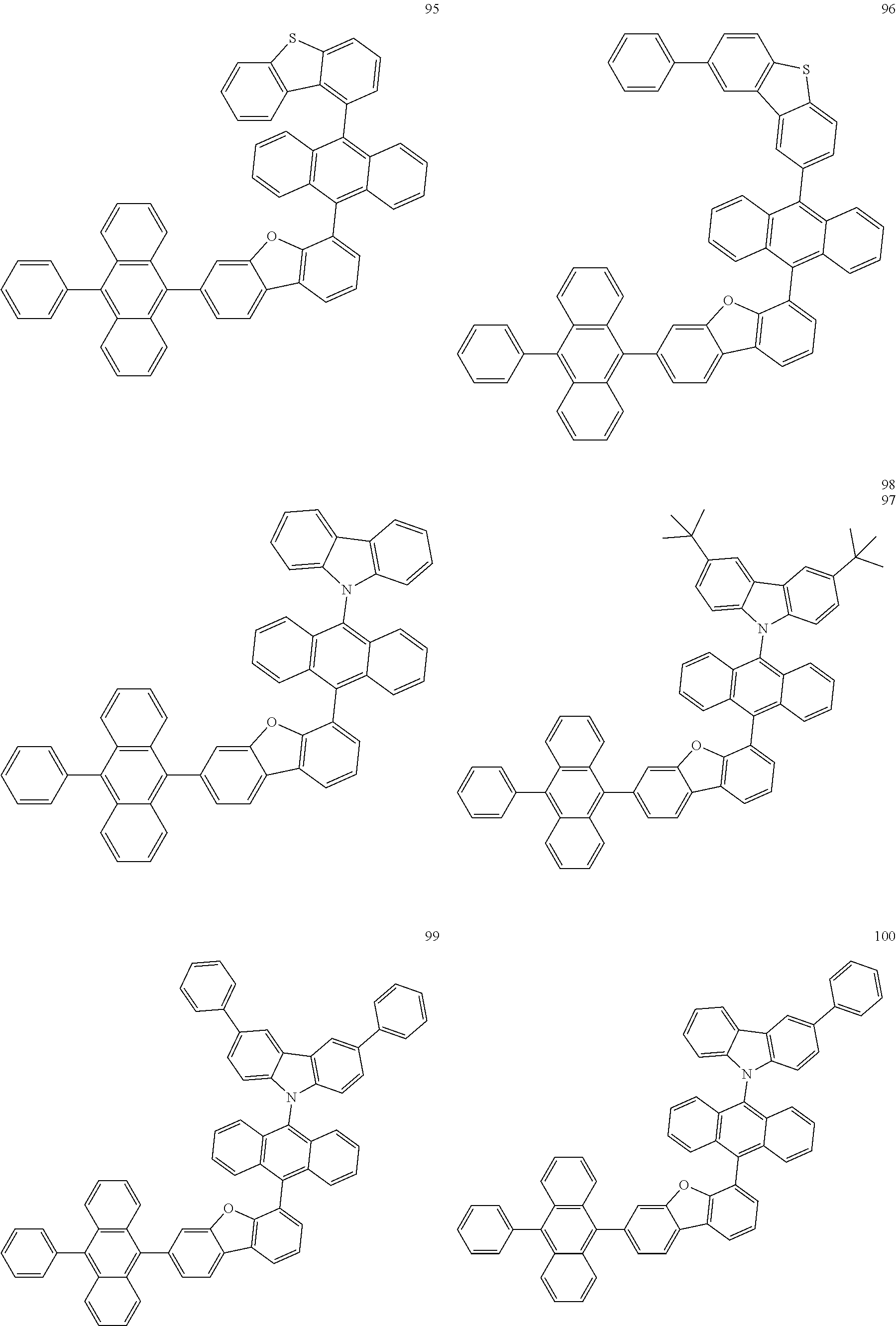

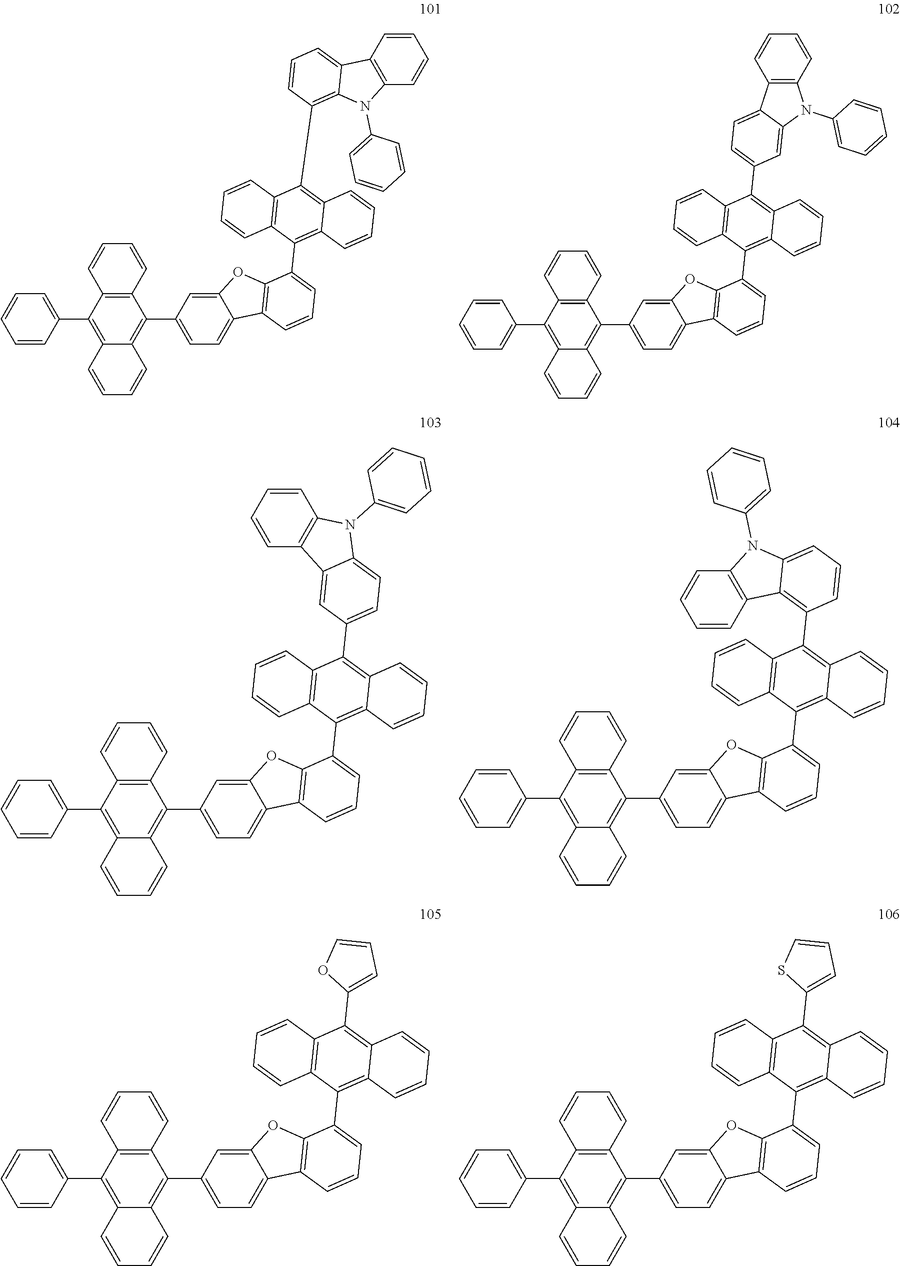

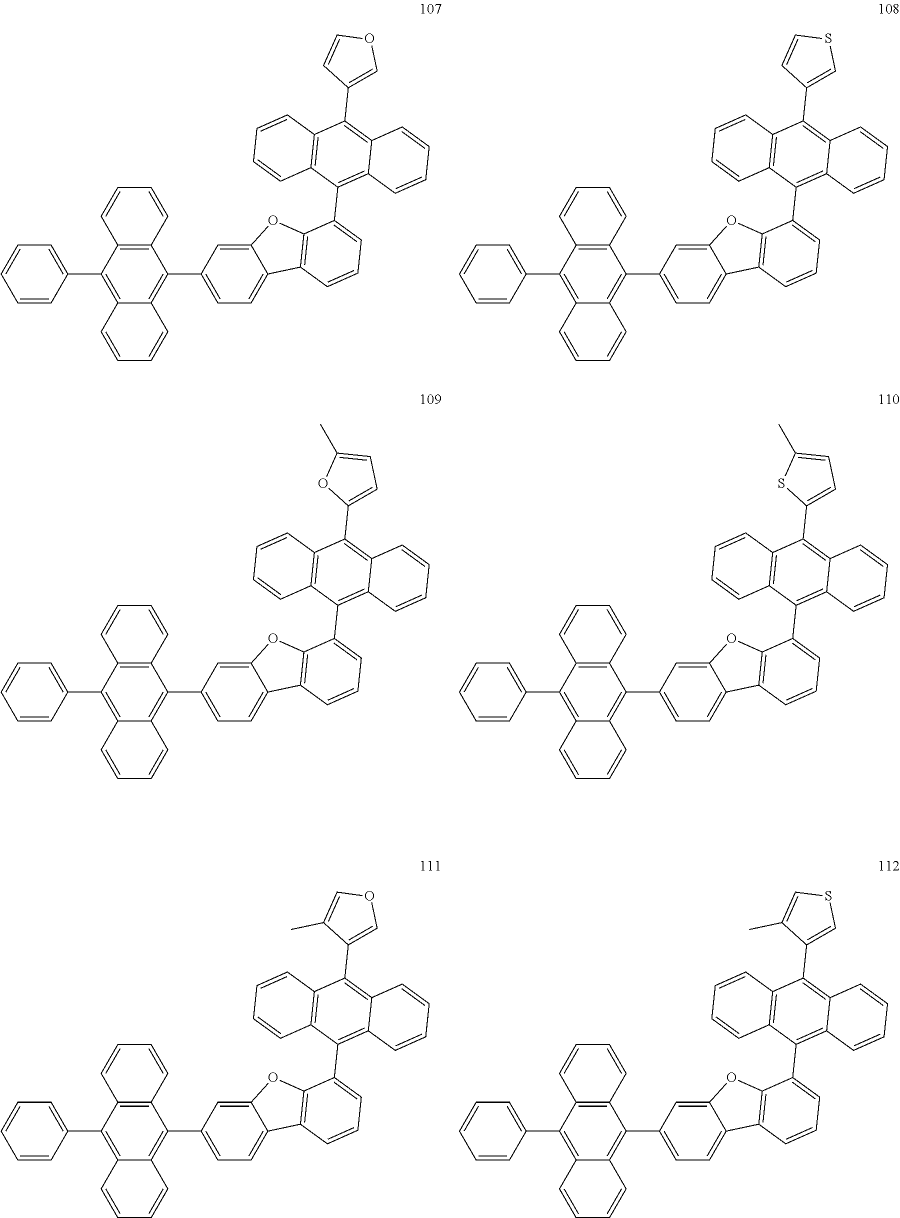

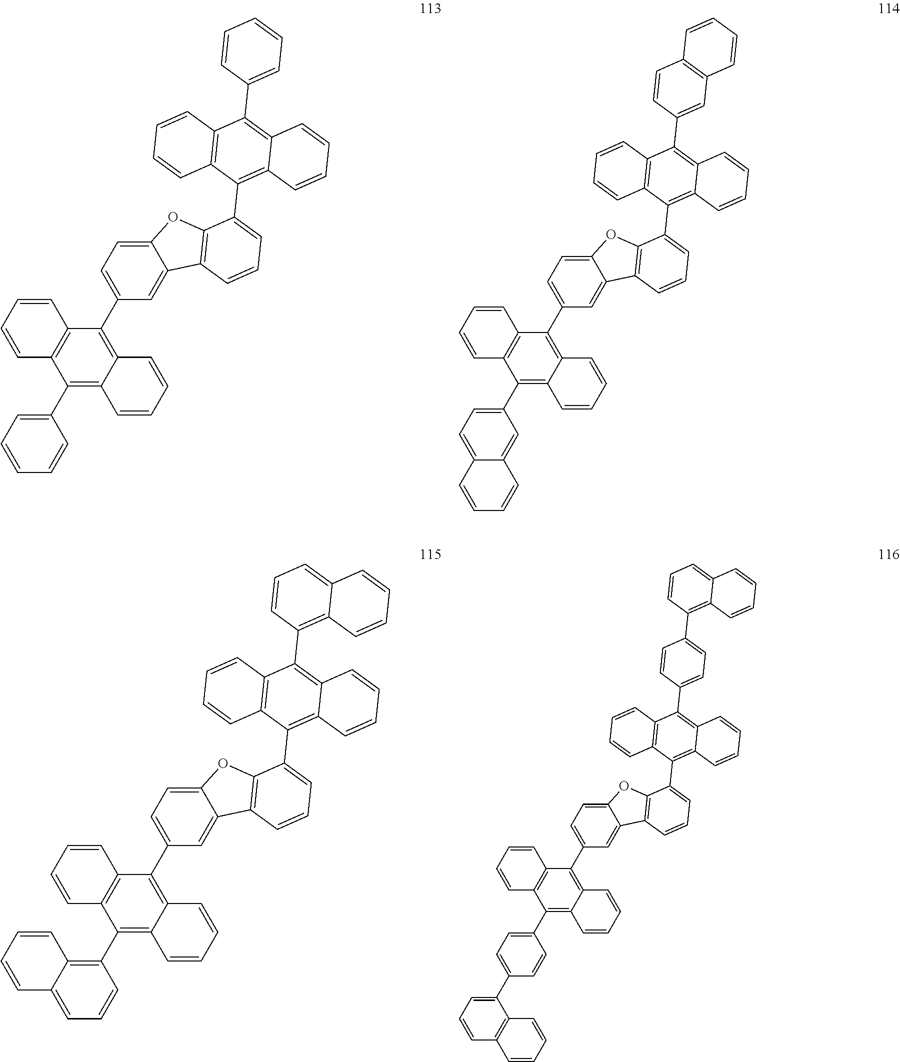 [SMILES: c1ccc(-c2c3ccccc3c(-c3ccc4oc5c(-c6c7ccccc7c(-c7ccccc7)c7ccccc67)cccc5c4c3)c3ccccc23)cc1 ; c1ccc2cc(-c3c4ccccc4c(-c4ccc5oc6c(-c7c8ccccc8c(-c8ccc9ccccc9c8)c8ccccc78)cccc6c5c4)c4ccccc34)ccc2c1 ; c1ccc2c(-c3c4ccccc4c(-c4ccc5oc6c(-c7c8ccccc8c(-c8cccc9ccccc89)c8ccccc78)cccc6c5c4)c4ccccc34)cccc2c1 ; c1ccc2c(-c3ccc(-c4c5ccccc5c(-c5ccc6oc7c(-c8c9ccccc9c(-c9ccc(-c%10cccc%11ccccc%10%11)cc9)c9ccccc89)cccc7c6c5)c5ccccc45)cc3)cccc2c1]

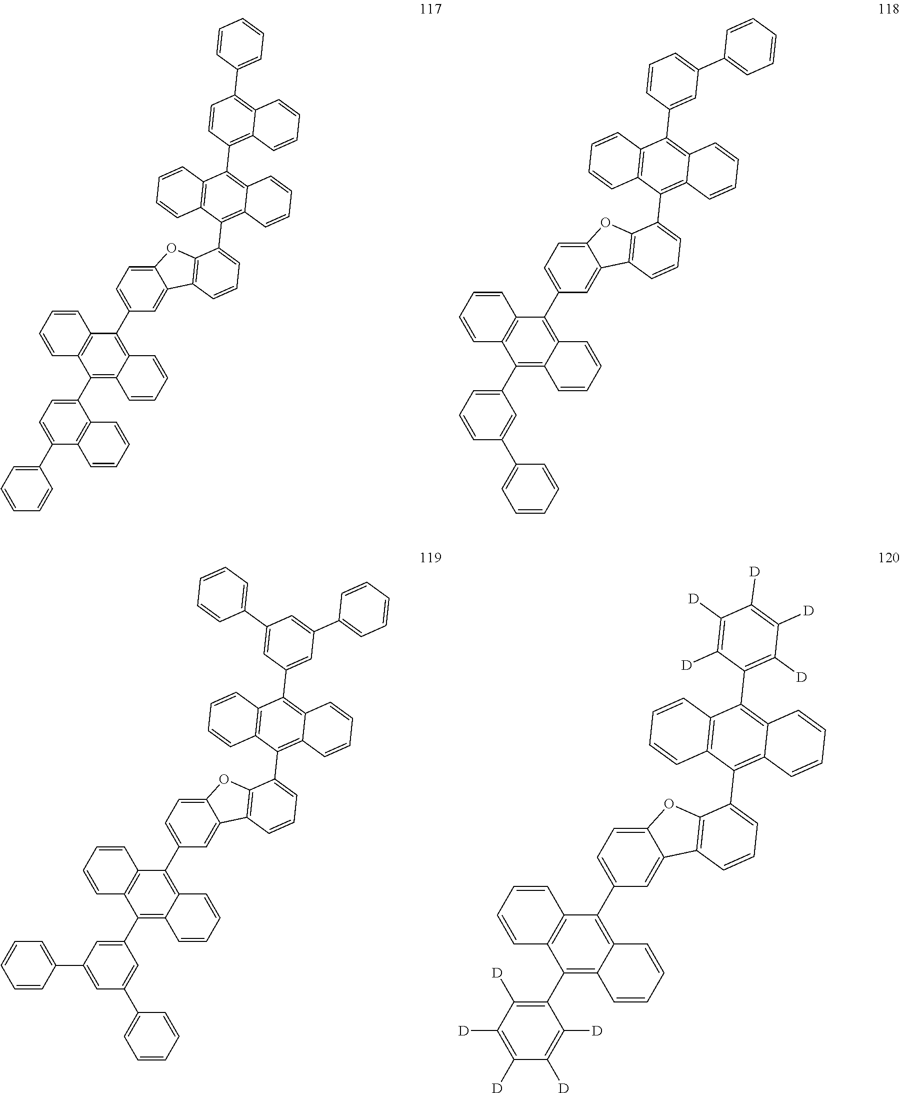

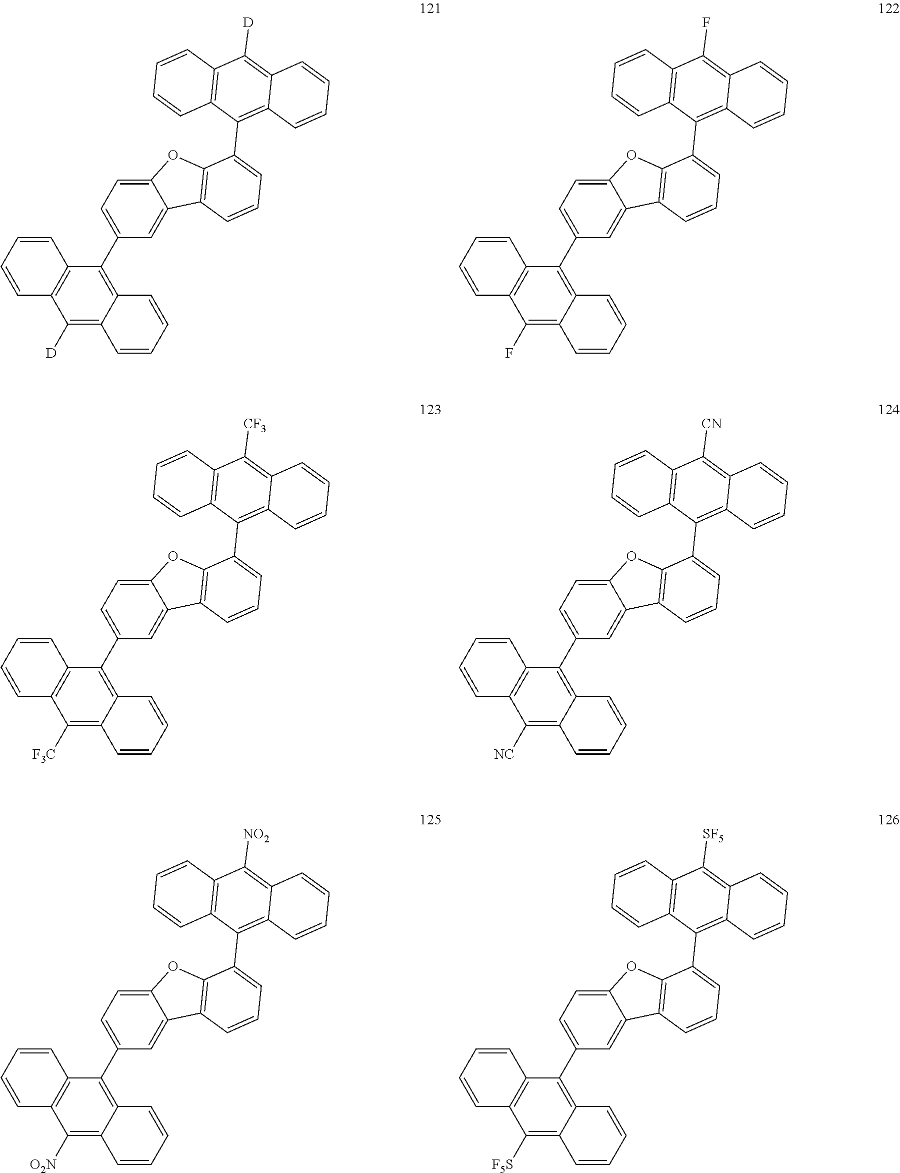

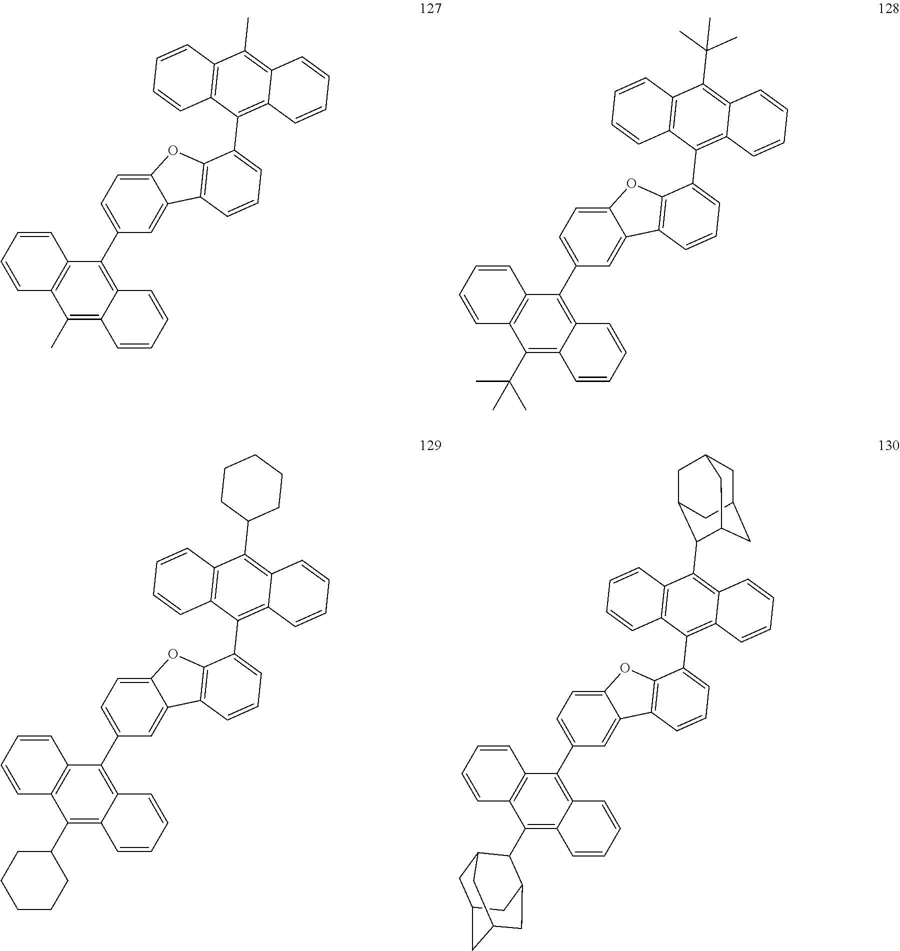 [SMILES: Cc1c2ccccc2c(-c2ccc3oc4c(-c5c6ccccc6c(C)c6ccccc56)cccc4c3c2)c2ccccc12 ; CC(C)(C)c1c2ccccc2c(-c2ccc3oc4c(-c5c6ccccc6c(C(C)(C)C)c6ccccc56)cccc4c3c2)c2ccccc12 ; c1ccc2c(C3CCCCC3)c3ccccc3c(-c3ccc4oc5c(-c6c7ccccc7c(C7CCCCC7)c7ccccc67)cccc5c4c3)c2c1 ; c1ccc2c(C3C4CC5CC(C4)CC3C5)c3ccccc3c(-c3ccc4oc5c(-c6c7ccccc7c(C7C8CC9CC(C8)CC7C9)c7ccccc67)cccc5c4c3)c2c1]

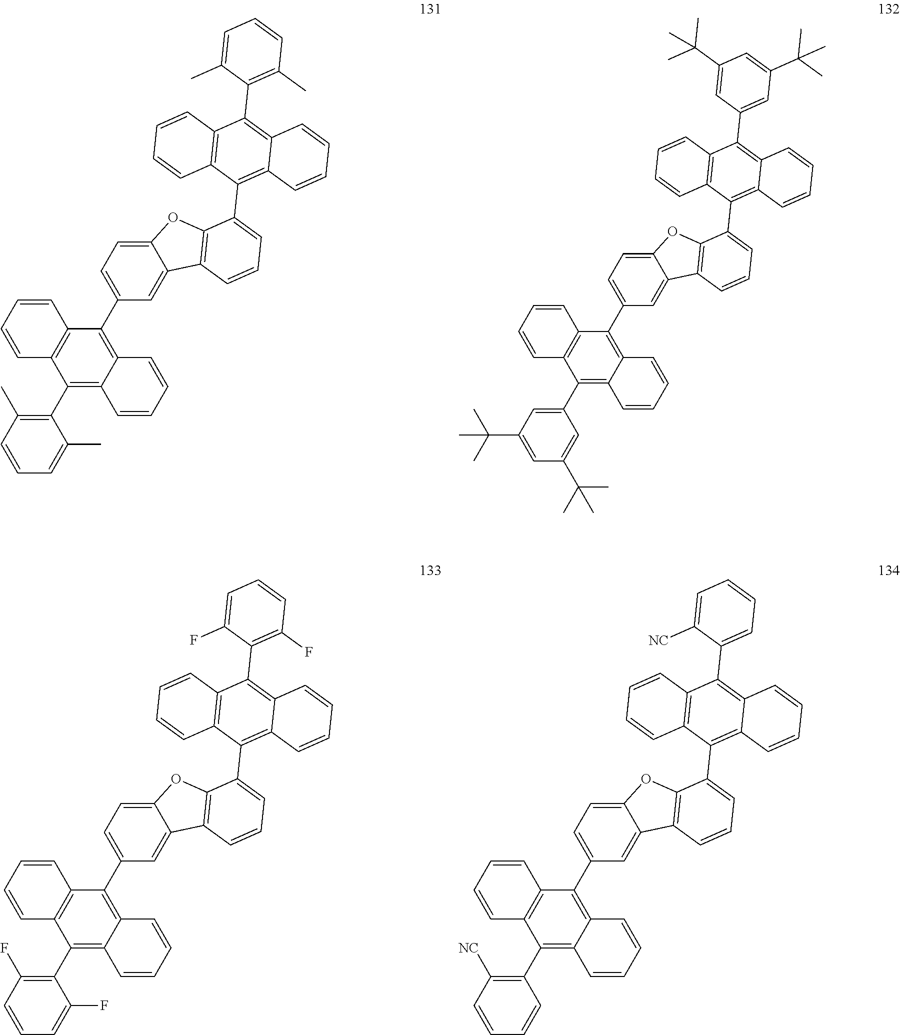 [SMILES: Cc1cccc(C)c1-c1c2ccccc2c(-c2ccc3oc4c(-c5c6ccccc6c(-c6c(C)cccc6C)c6ccccc56)cccc4c3c2)c2ccccc12 ; CC(C)(C)c1cc(-c2c3ccccc3c(-c3ccc4oc5c(-c6c7ccccc7c(-c7cc(C(C)(C)C)cc(C(C)(C)C)c7)c7ccccc67)cccc5c4c3)c3ccccc23)cc(C(C)(C)C)c1 ; Fc1cccc(F)c1-c1c2ccccc2c(-c2ccc3oc4c(-c5c6ccccc6c(-c6c(F)cccc6F)c6ccccc56)cccc4c3c2)c2ccccc12 ; N#Cc1ccccc1-c1c2ccccc2c(-c2ccc3oc4c(-c5c6ccccc6c(-c6ccccc6C#N)c6ccccc56)cccc4c3c2)c2ccccc12]

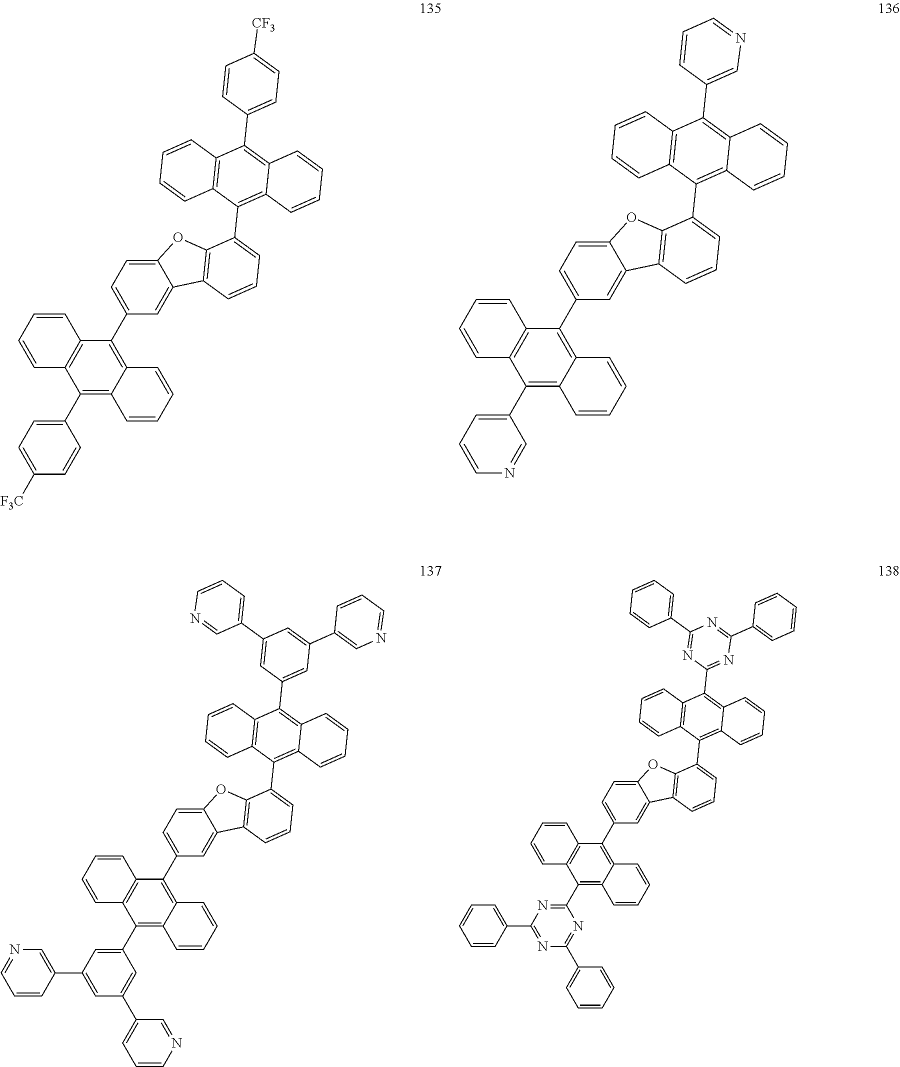 [SMILES: FC(F)(F)c1ccc(-c2c3ccccc3c(-c3ccc4oc5c(-c6c7ccccc7c(-c7ccc(C(F)(F)F)cc7)c7ccccc67)cccc5c4c3)c3ccccc23)cc1 ; c1cncc(-c2c3ccccc3c(-c3ccc4oc5c(-c6c7ccccc7c(-c7cccnc7)c7ccccc67)cccc5c4c3)c3ccccc23)c1 ; c1cncc(-c2cc(-c3cccnc3)cc(-c3c4ccccc4c(-c4ccc5oc6c(-c7c8ccccc8c(-c8cc(-c9cccnc9)cc(-c9cccnc9)c8)c8ccccc78)cccc6c5c4)c4ccccc34)c2)c1 ; c1ccc(-c2nc(-c3ccccc3)nc(-c3c4ccccc4c(-c4ccc5oc6c(-c7c8ccccc8c(-c8nc(-c9ccccc9)nc(-c9ccccc9)n8)c8ccccc78)cccc6c5c4)c4ccccc34)n2)cc1]

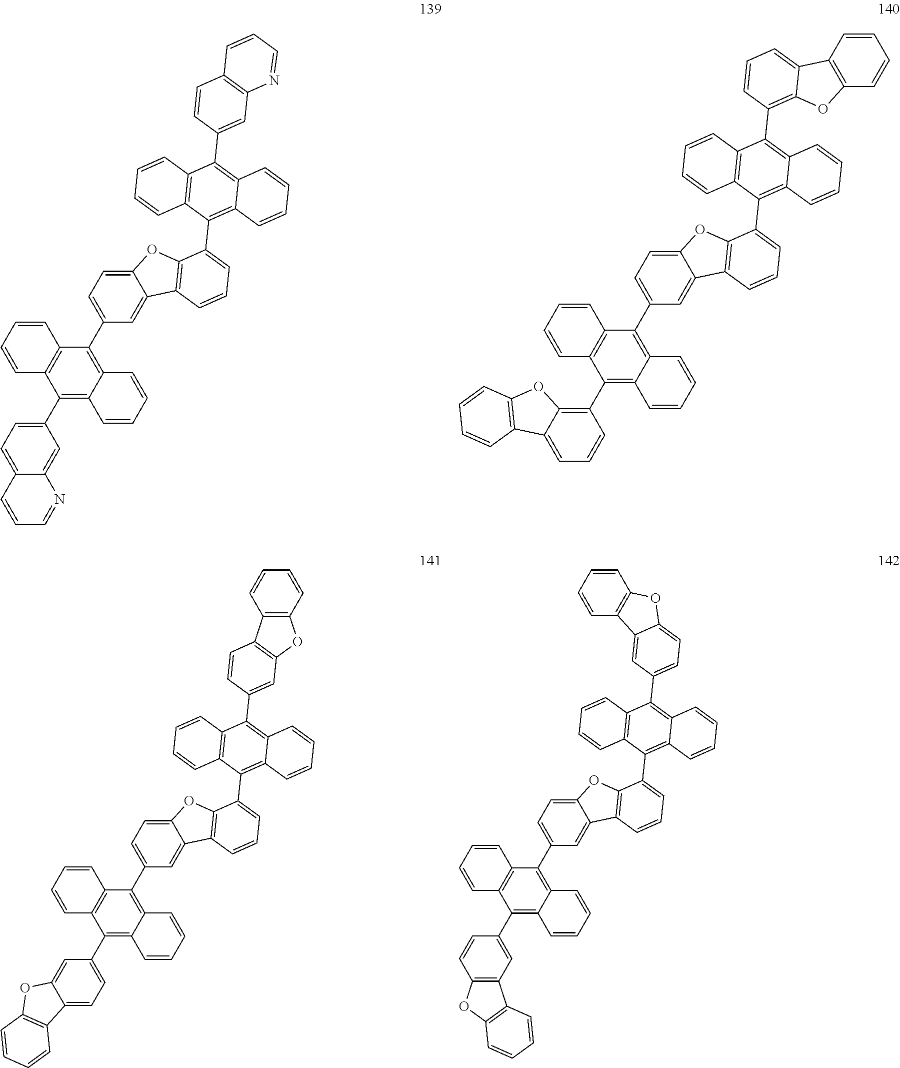 [SMILES: c1cnc2cc(-c3c4ccccc4c(-c4ccc5oc6c(-c7c8ccccc8c(-c8ccc9cccnc9c8)c8ccccc78)cccc6c5c4)c4ccccc34)ccc2c1 ; c1ccc2c(c1)oc1c(-c3c4ccccc4c(-c4ccc5oc6c(-c7c8ccccc8c(-c8cccc9c8oc8ccccc89)c8ccccc78)cccc6c5c4)c4ccccc34)cccc12 ; c1ccc2c(c1)oc1cc(-c3c4ccccc4c(-c4ccc5oc6c(-c7c8ccccc8c(-c8ccc9c(c8)oc8ccccc89)c8ccccc78)cccc6c5c4)c4ccccc34)ccc12 ; c1ccc2c(c1)oc1ccc(-c3c4ccccc4c(-c4ccc5oc6c(-c7c8ccccc8c(-c8ccc9oc%10ccccc%10c9c8)c8ccccc78)cccc6c5c4)c4ccccc34)cc12]

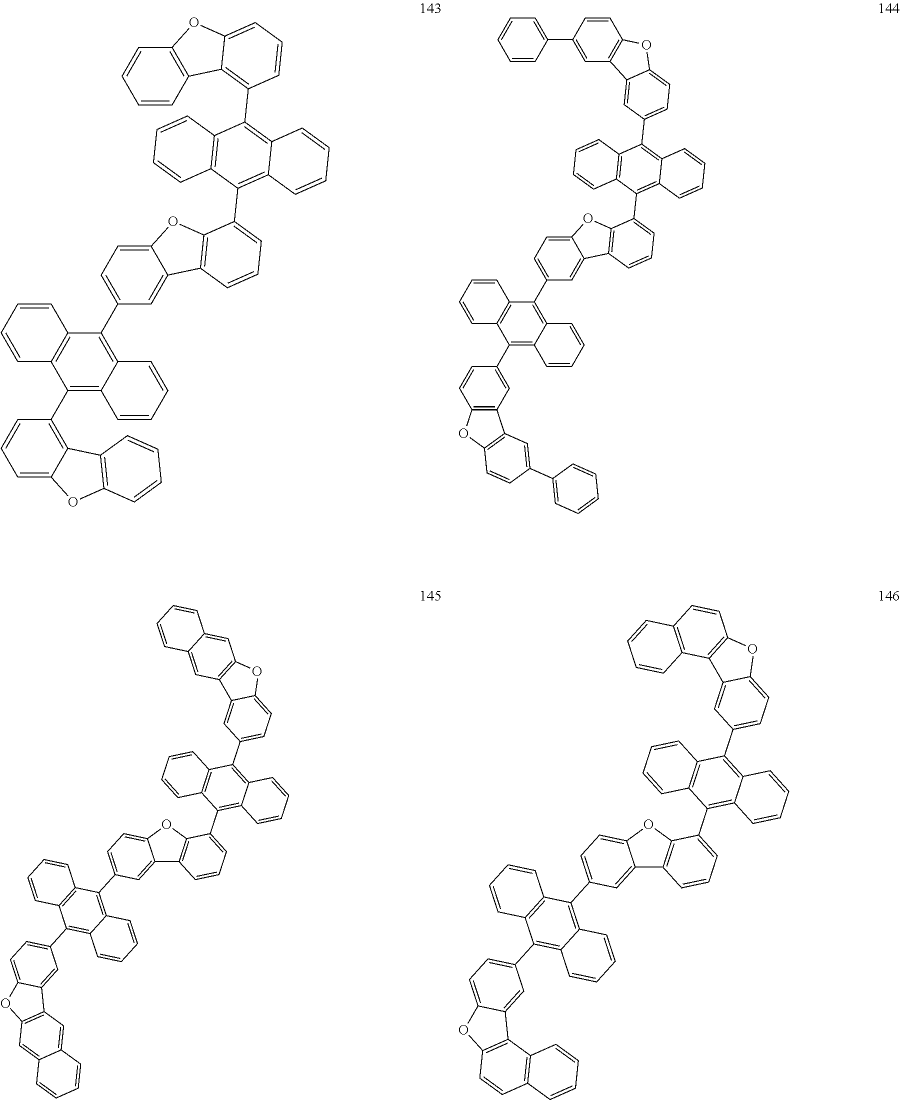

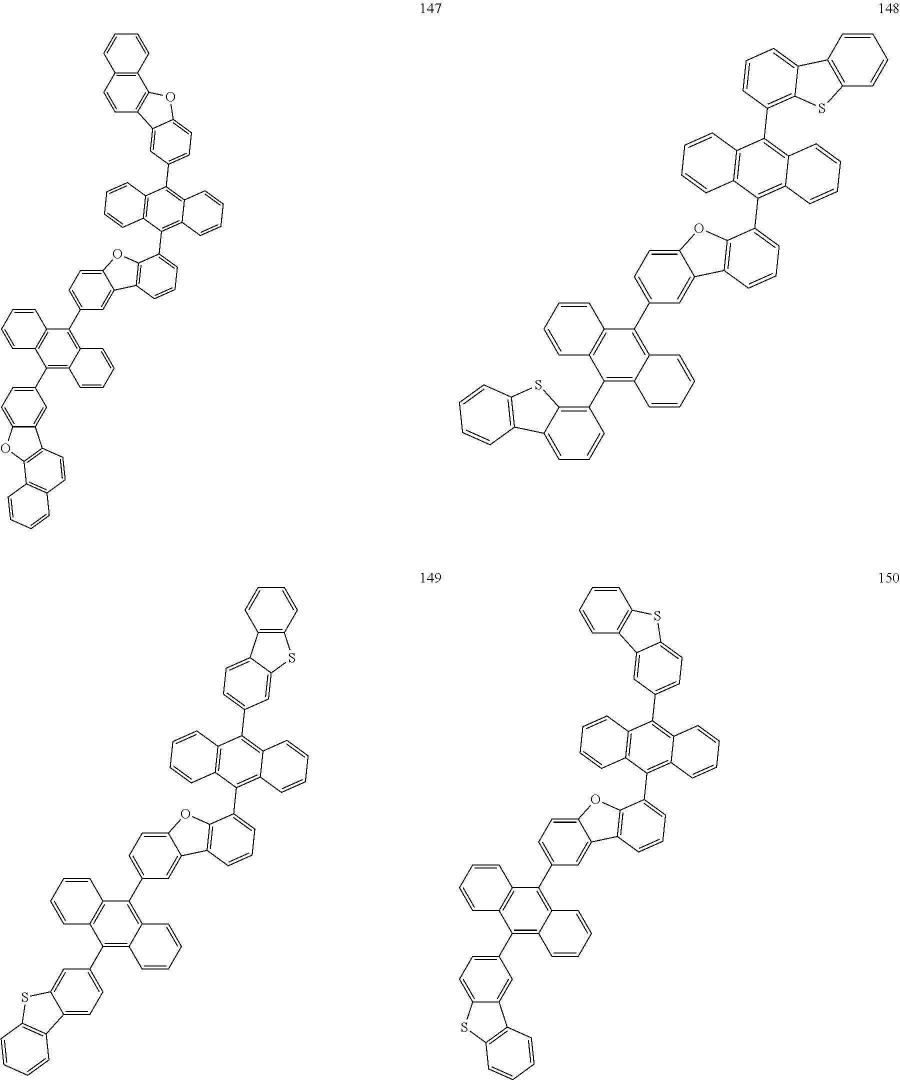 [SMILES: c1ccc2c(c1)ccc1c3cc(-c4c5ccccc5c(-c5ccc6oc7c(-c8c9ccccc9c(-c9ccc%10oc%11c%12ccccc%12ccc%11c%10c9)c9ccccc89)cccc7c6c5)c5ccccc45)ccc3oc21 ; c1ccc2c(c1)sc1c(-c3c4ccccc4c(-c4ccc5oc6c(-c7c8ccccc8c(-c8cccc9c8sc8ccccc89)c8ccccc78)cccc6c5c4)c4ccccc34)cccc12 ; c1ccc2c(c1)sc1cc(-c3c4ccccc4c(-c4ccc5oc6c(-c7c8ccccc8c(-c8ccc9c(c8)sc8ccccc89)c8ccccc78)cccc6c5c4)c4ccccc34)ccc12 ; c1ccc2c(c1)sc1ccc(-c3c4ccccc4c(-c4ccc5oc6c(-c7c8ccccc8c(-c8ccc9sc%10ccccc%10c9c8)c8ccccc78)cccc6c5c4)c4ccccc34)cc12]

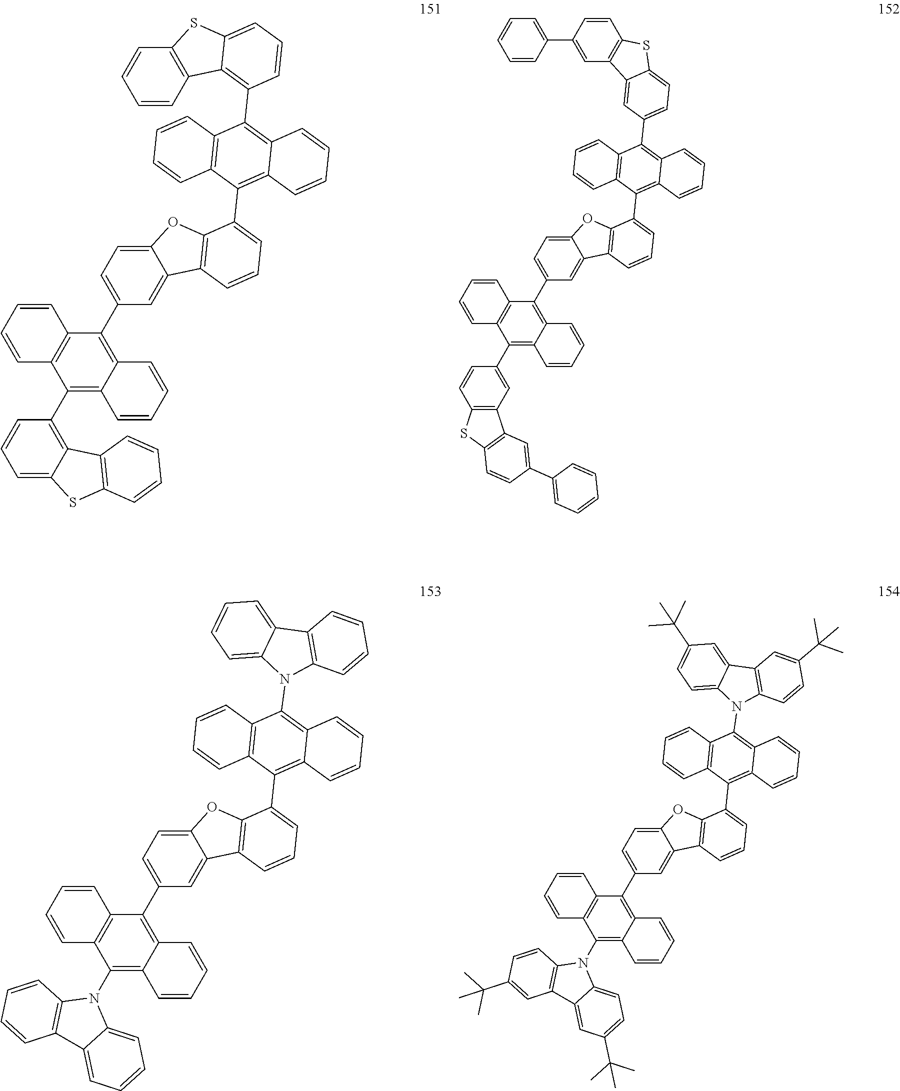

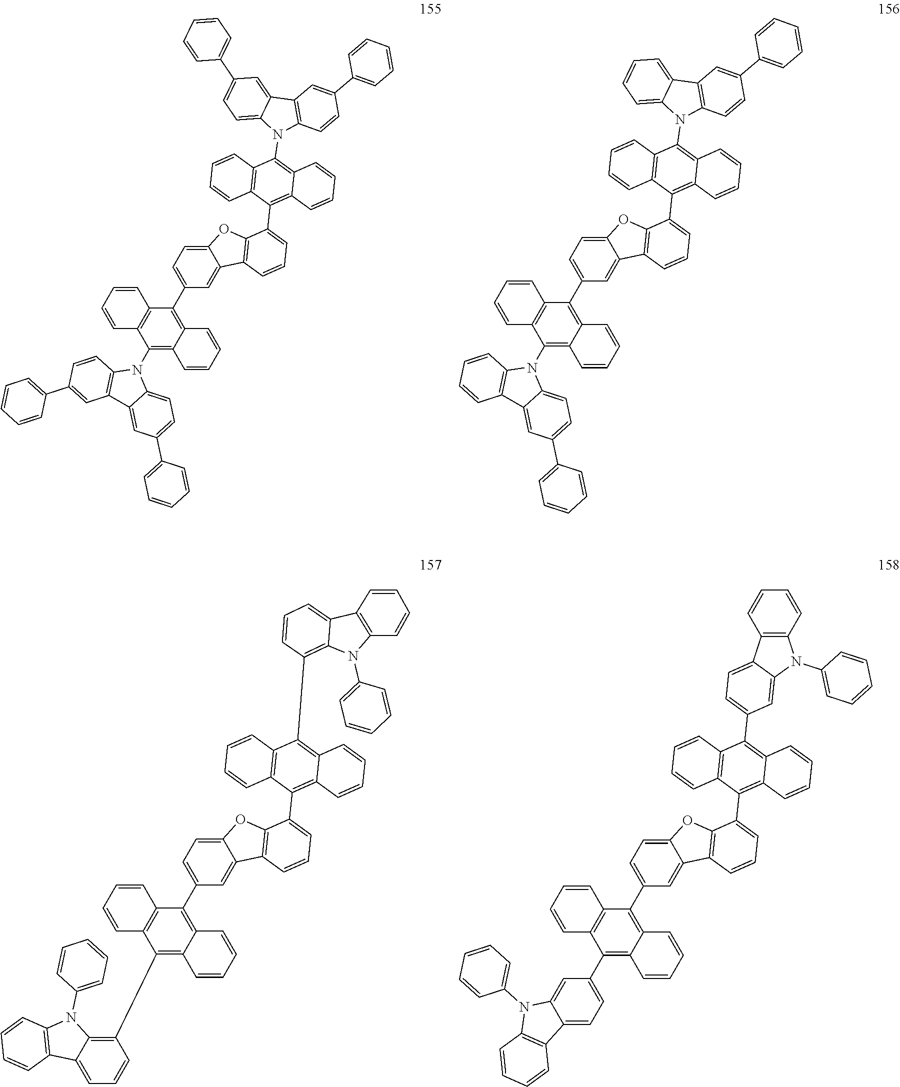

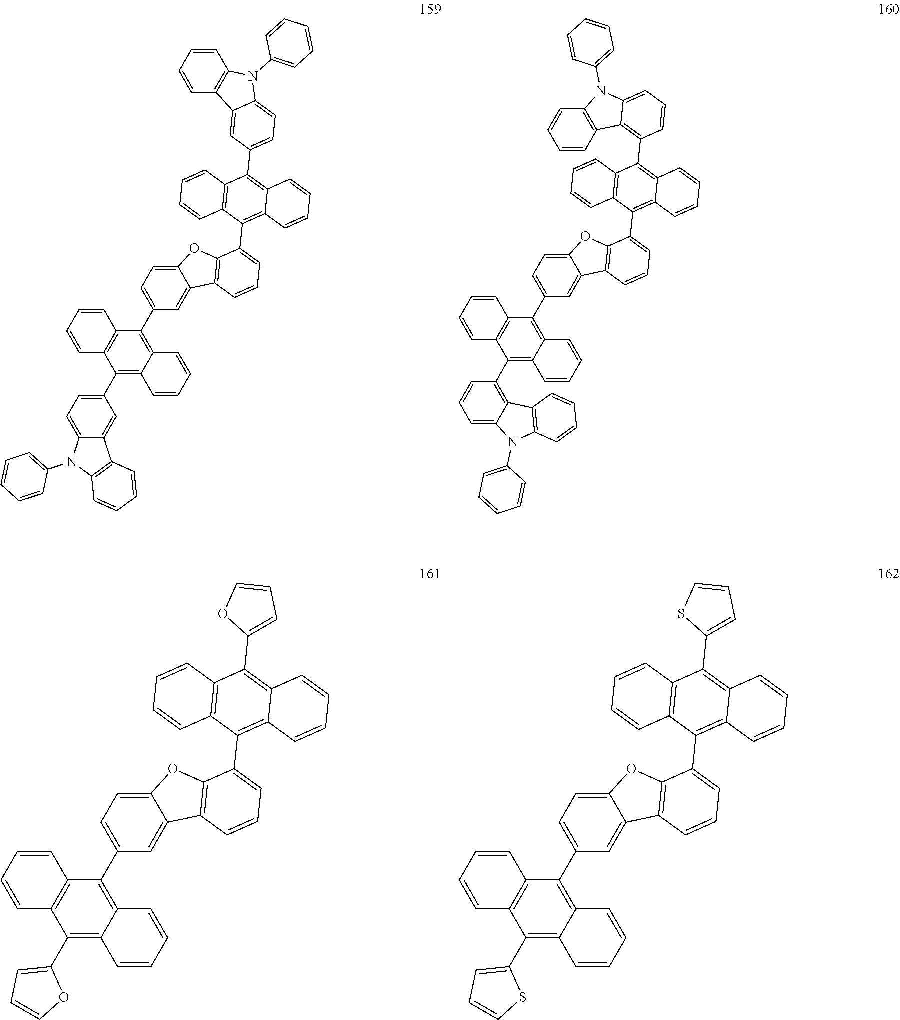 [SMILES: c1ccc(-n2c3ccccc3c3cc(-c4c5ccccc5c(-c5ccc6oc7c(-c8c9ccccc9c(-c9ccc%10c(c9)c9ccccc9n%10-c9ccccc9)c9ccccc89)cccc7c6c5)c5ccccc45)ccc32)cc1 ; c1ccc(-n2c3ccccc3c3c(-c4c5ccccc5c(-c5ccc6oc7c(-c8c9ccccc9c(-c9cccc%10c9c9ccccc9n%10-c9ccccc9)c9ccccc89)cccc7c6c5)c5ccccc45)cccc32)cc1 ; c1coc(-c2c3ccccc3c(-c3ccc4oc5c(-c6c7ccccc7c(-c7ccco7)c7ccccc67)cccc5c4c3)c3ccccc23)c1 ; c1csc(-c2c3ccccc3c(-c3ccc4oc5c(-c6c7ccccc7c(-c7cccs7)c7ccccc67)cccc5c4c3)c3ccccc23)c1]

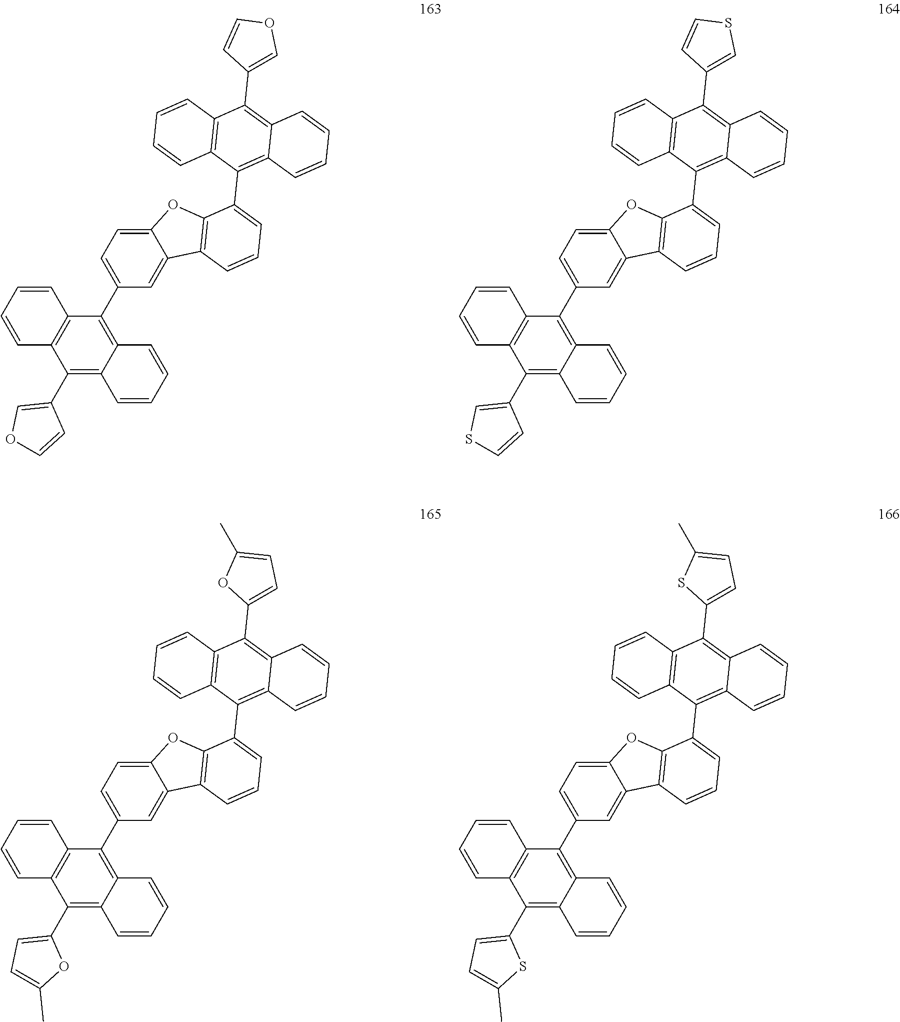 [SMILES: c1ccc2c(-c3ccc4oc5c(-c6c7ccccc7c(-c7ccoc7)c7ccccc67)cccc5c4c3)c3ccccc3c(-c3ccoc3)c2c1 ; c1ccc2c(-c3ccc4oc5c(-c6c7ccccc7c(-c7ccsc7)c7ccccc67)cccc5c4c3)c3ccccc3c(-c3ccsc3)c2c1 ; Cc1ccc(-c2c3ccccc3c(-c3ccc4oc5c(-c6c7ccccc7c(-c7ccc(C)o7)c7ccccc67)cccc5c4c3)c3ccccc23)o1 ; Cc1ccc(-c2c3ccccc3c(-c3ccc4oc5c(-c6c7ccccc7c(-c7ccc(C)s7)c7ccccc67)cccc5c4c3)c3ccccc23)s1]

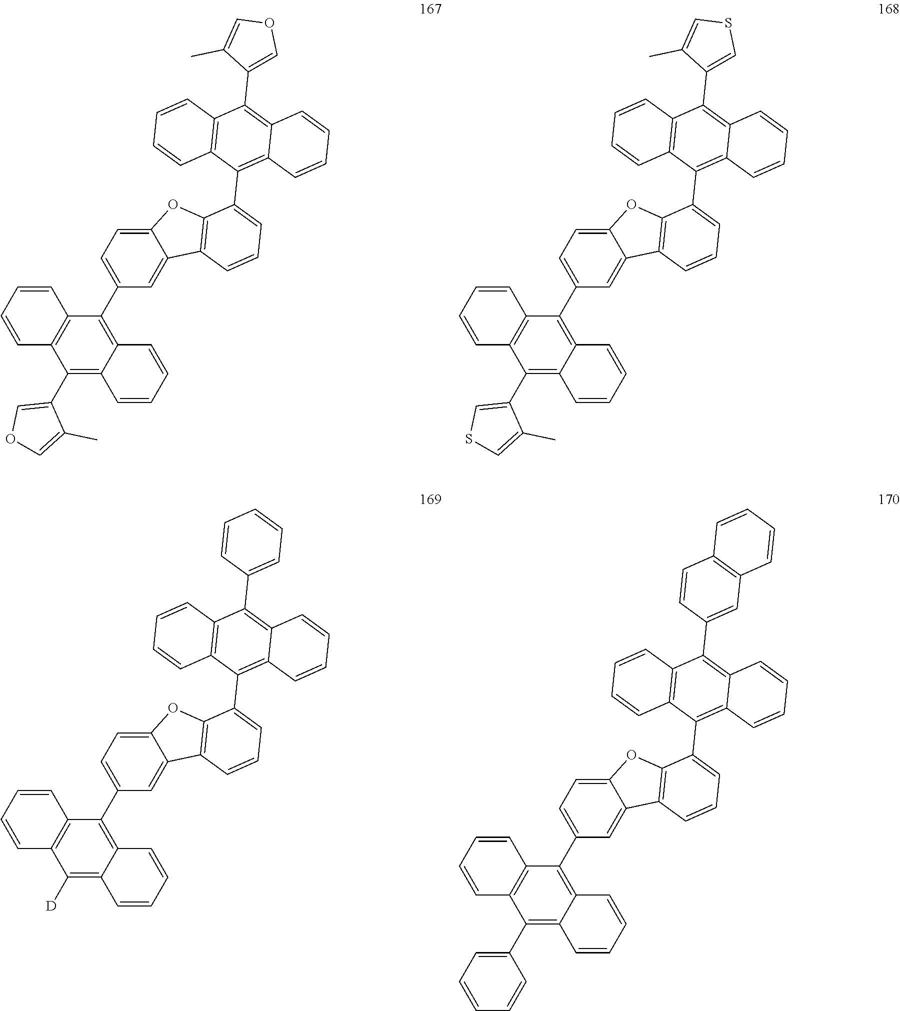 [SMILES: Cc1cocc1-c1c2ccccc2c(-c2ccc3oc4c(-c5c6ccccc6c(-c6cocc6C)c6ccccc56)cccc4c3c2)c2ccccc12 ; Cc1cscc1-c1c2ccccc2c(-c2ccc3oc4c(-c5c6ccccc6c(-c6cscc6C)c6ccccc56)cccc4c3c2)c2ccccc12 ; [2H]c1c2ccccc2c(-c2ccc3oc4c(-c5c6ccccc6c(-c6ccccc6)c6ccccc56)cccc4c3c2)c2ccccc12 ; c1ccc(-c2c3ccccc3c(-c3ccc4oc5c(-c6c7ccccc7c(-c7ccc8ccccc8c7)c7ccccc67)cccc5c4c3)c3ccccc23)cc1]

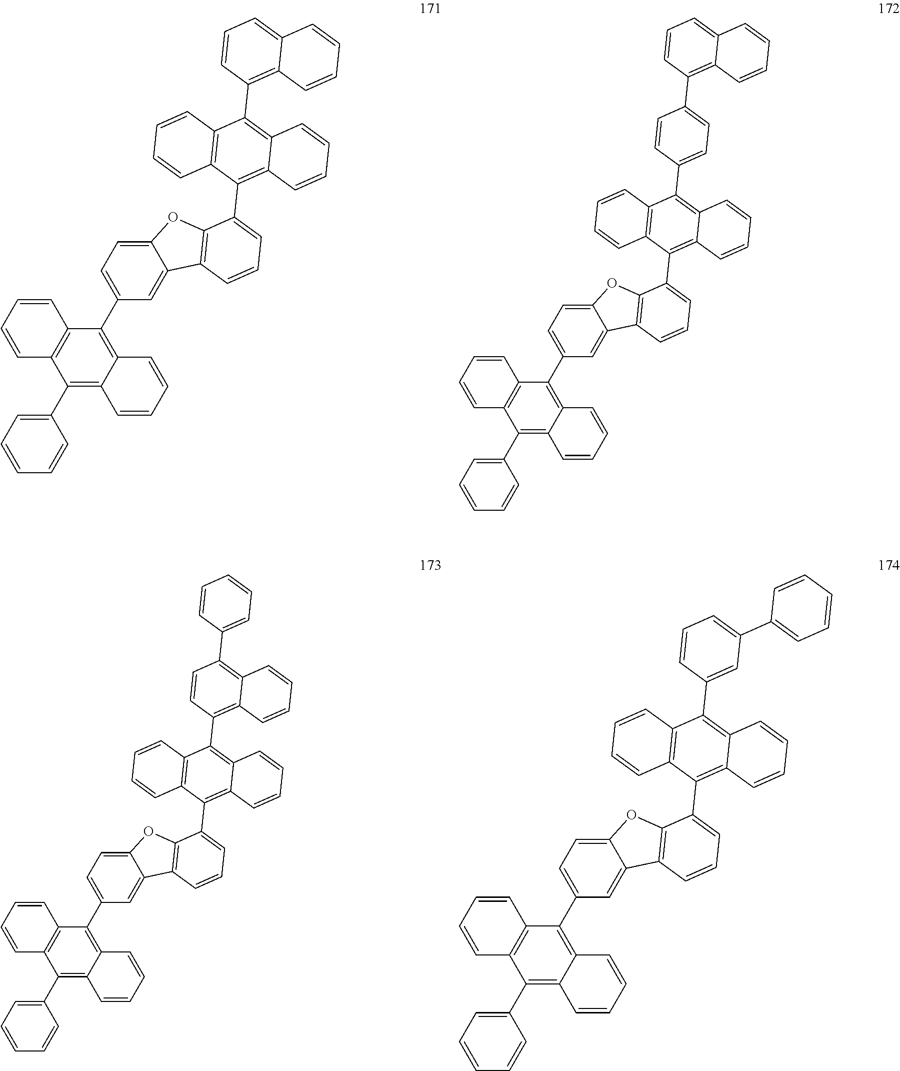 [SMILES: c1ccc(-c2c3ccccc3c(-c3ccc4oc5c(-c6c7ccccc7c(-c7cccc8ccccc78)c7ccccc67)cccc5c4c3)c3ccccc23)cc1 ; c1ccc(-c2c3ccccc3c(-c3ccc4oc5c(-c6c7ccccc7c(-c7ccc(-c8cccc9ccccc89)cc7)c7ccccc67)cccc5c4c3)c3ccccc23)cc1 ; c1ccc(-c2ccc(-c3c4ccccc4c(-c4cccc5c4oc4ccc(-c6c7ccccc7c(-c7ccccc7)c7ccccc67)cc45)c4ccccc34)c3ccccc23)cc1 ; c1ccc(-c2cccc(-c3c4ccccc4c(-c4cccc5c4oc4ccc(-c6c7ccccc7c(-c7ccccc7)c7ccccc67)cc45)c4ccccc34)c2)cc1]

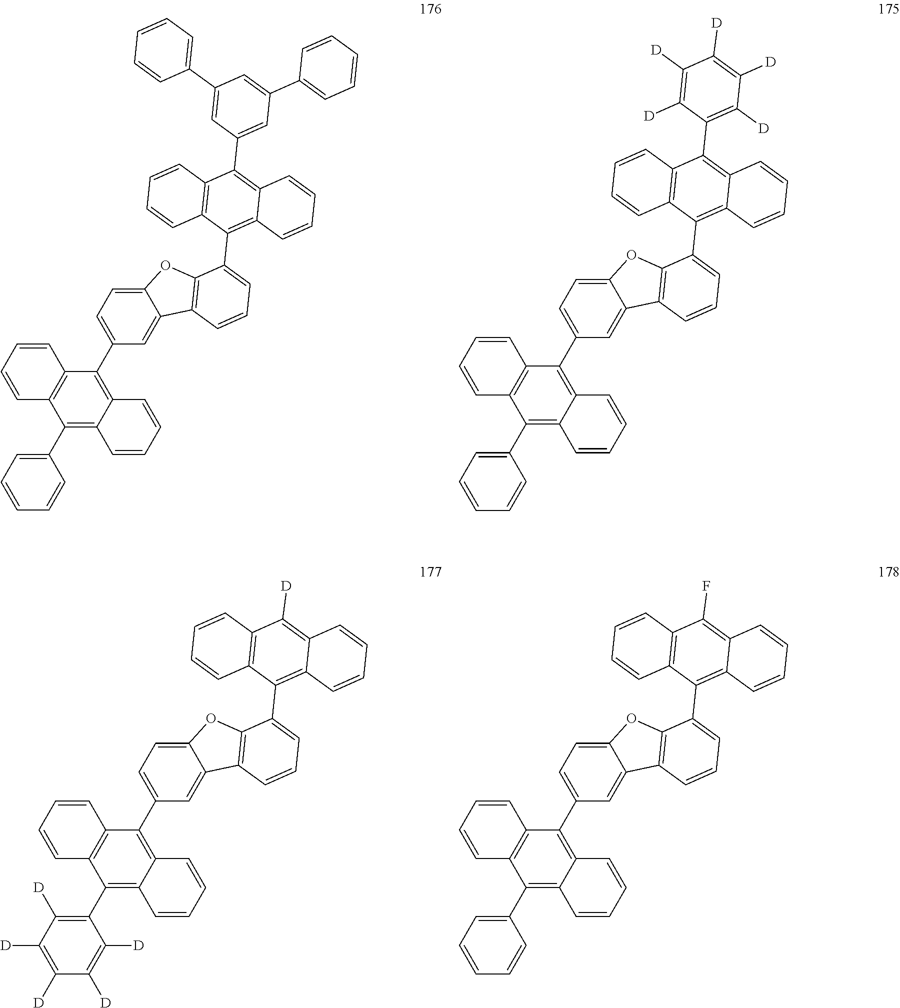 [SMILES: c1ccc(-c2cc(-c3ccccc3)cc(-c3c4ccccc4c(-c4cccc5c4oc4ccc(-c6c7ccccc7c(-c7ccccc7)c7ccccc67)cc45)c4ccccc34)c2)cc1 ; [2H]c1c([2H])c([2H])c(-c2c3ccccc3c(-c3cccc4c3oc3ccc(-c5c6ccccc6c(-c6ccccc6)c6ccccc56)cc34)c3ccccc23)c([2H])c1[2H] ; [2H]c1c([2H])c([2H])c(-c2c3ccccc3c(-c3ccc4oc5c(-c6c7ccccc7c([2H])c7ccccc67)cccc5c4c3)c3ccccc23)c([2H])c1[2H] ; Fc1c2ccccc2c(-c2cccc3c2oc2ccc(-c4c5ccccc5c(-c5ccccc5)c5ccccc45)cc23)c2ccccc12]

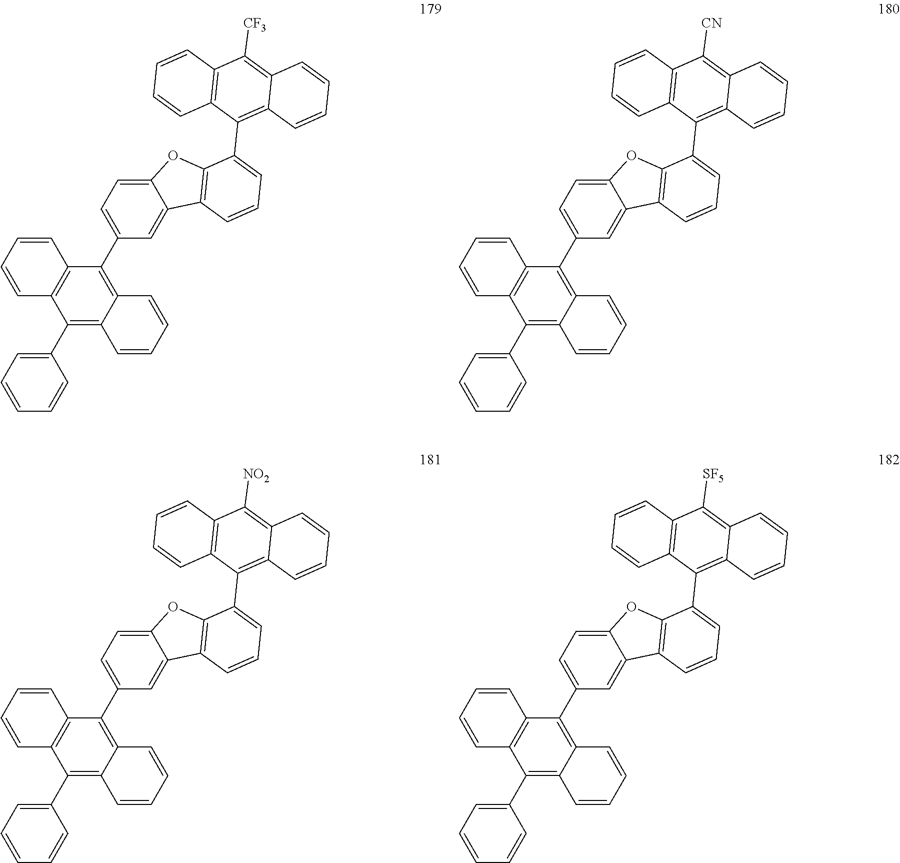

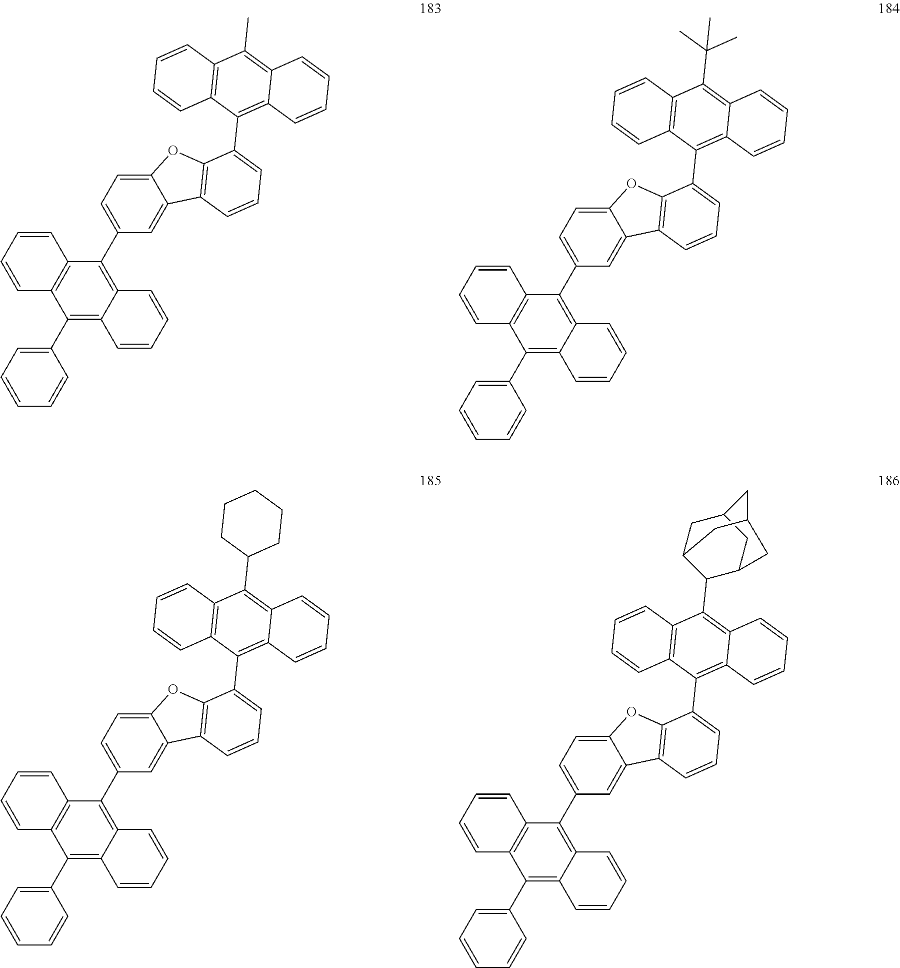 [SMILES: Cc1c2ccccc2c(-c2cccc3c2oc2ccc(-c4c5ccccc5c(-c5ccccc5)c5ccccc45)cc23)c2ccccc12 ; CC(C)(C)c1c2ccccc2c(-c2cccc3c2oc2ccc(-c4c5ccccc5c(-c5ccccc5)c5ccccc45)cc23)c2ccccc12 ; c1ccc(-c2c3ccccc3c(-c3ccc4oc5c(-c6c7ccccc7c(C7CCCCC7)c7ccccc67)cccc5c4c3)c3ccccc23)cc1 ; c1ccc(-c2c3ccccc3c(-c3ccc4oc5c(-c6c7ccccc7c(C7C8CC9CC(C8)CC7C9)c7ccccc67)cccc5c4c3)c3ccccc23)cc1]

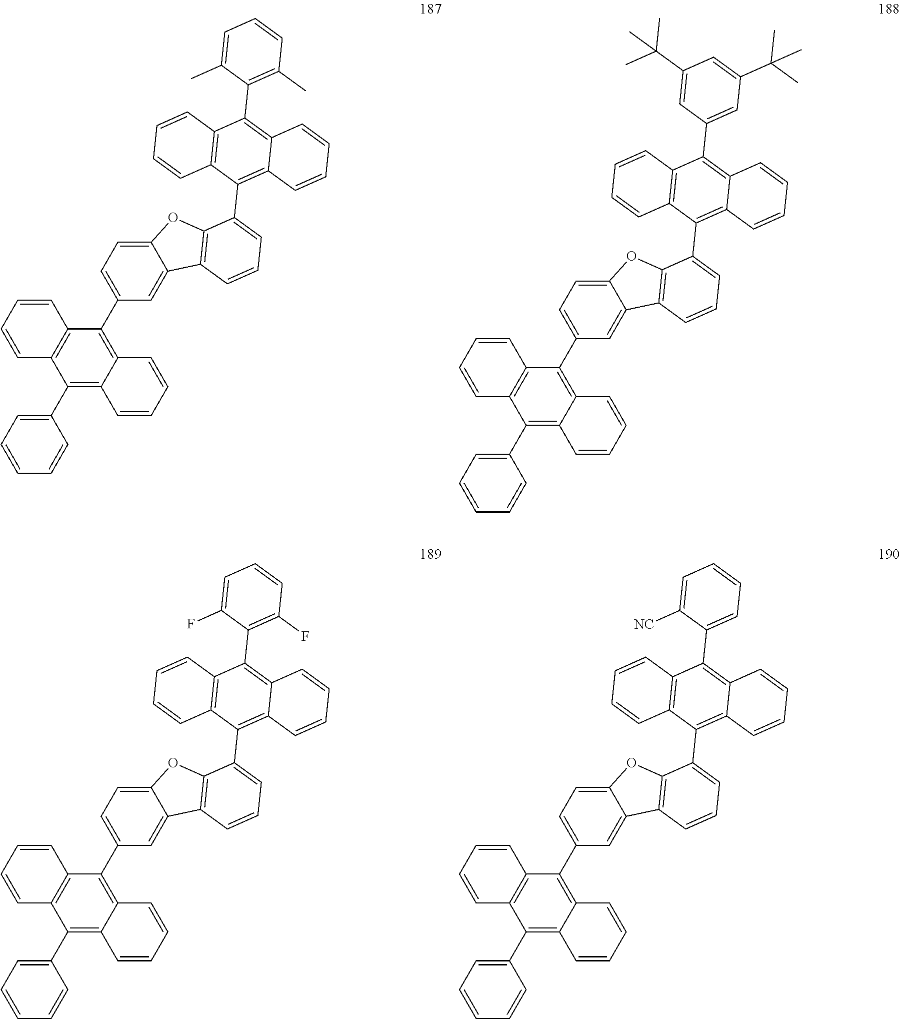 [SMILES: Cc1cccc(C)c1-c1c2ccccc2c(-c2cccc3c2oc2ccc(-c4c5ccccc5c(-c5ccccc5)c5ccccc45)cc23)c2ccccc12 ; CC(C)(C)c1cc(-c2c3ccccc3c(-c3cccc4c3oc3ccc(-c5c6ccccc6c(-c6ccccc6)c6ccccc56)cc34)c3ccccc23)cc(C(C)(C)C)c1 ; Fc1cccc(F)c1-c1c2ccccc2c(-c2cccc3c2oc2ccc(-c4c5ccccc5c(-c5ccccc5)c5ccccc45)cc23)c2ccccc12 ; N#Cc1ccccc1-c1c2ccccc2c(-c2cccc3c2oc2ccc(-c4c5ccccc5c(-c5ccccc5)c5ccccc45)cc23)c2ccccc12]

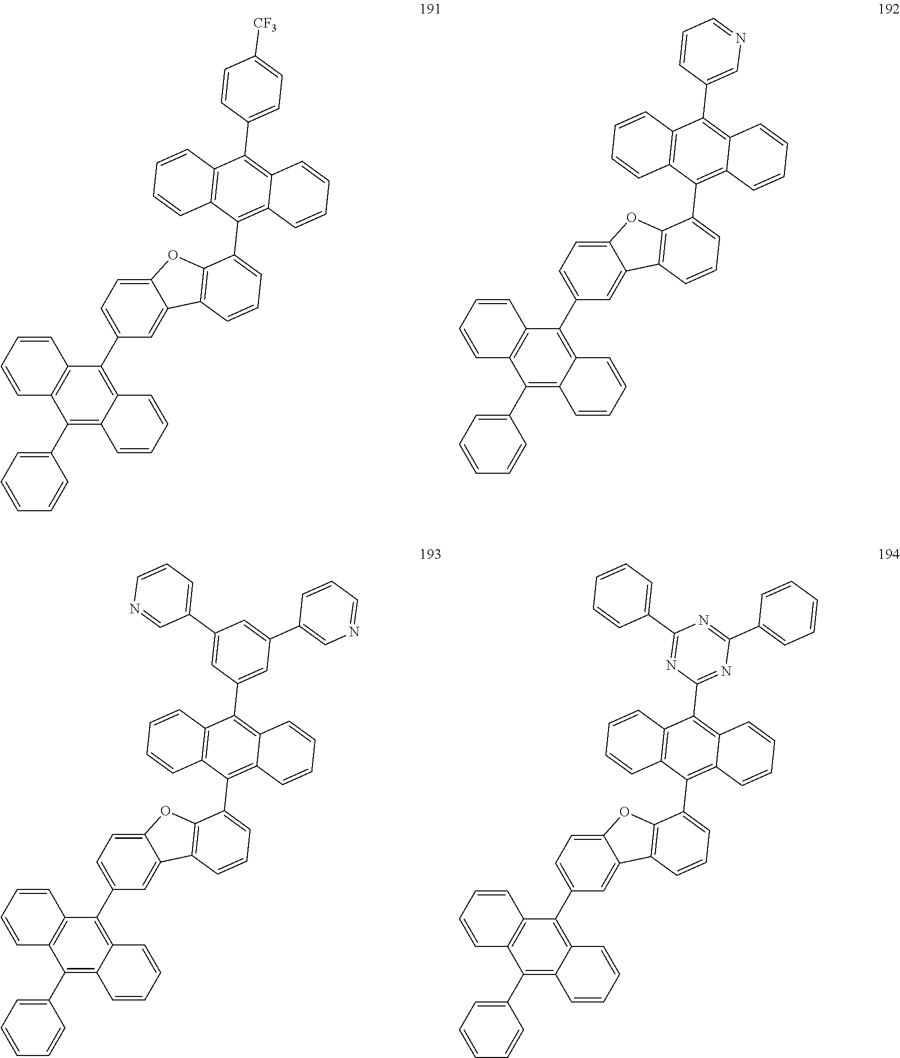 [SMILES: FC(F)(F)c1ccc(-c2c3ccccc3c(-c3cccc4c3oc3ccc(-c5c6ccccc6c(-c6ccccc6)c6ccccc56)cc34)c3ccccc23)cc1 ; c1ccc(-c2c3ccccc3c(-c3ccc4oc5c(-c6c7ccccc7c(-c7cccnc7)c7ccccc67)cccc5c4c3)c3ccccc23)cc1 ; c1ccc(-c2c3ccccc3c(-c3ccc4oc5c(-c6c7ccccc7c(-c7cc(-c8cccnc8)cc(-c8cccnc8)c7)c7ccccc67)cccc5c4c3)c3ccccc23)cc1 ; c1ccc(-c2nc(-c3ccccc3)nc(-c3c4ccccc4c(-c4cccc5c4oc4ccc(-c6c7ccccc7c(-c7ccccc7)c7ccccc67)cc45)c4ccccc34)n2)cc1]

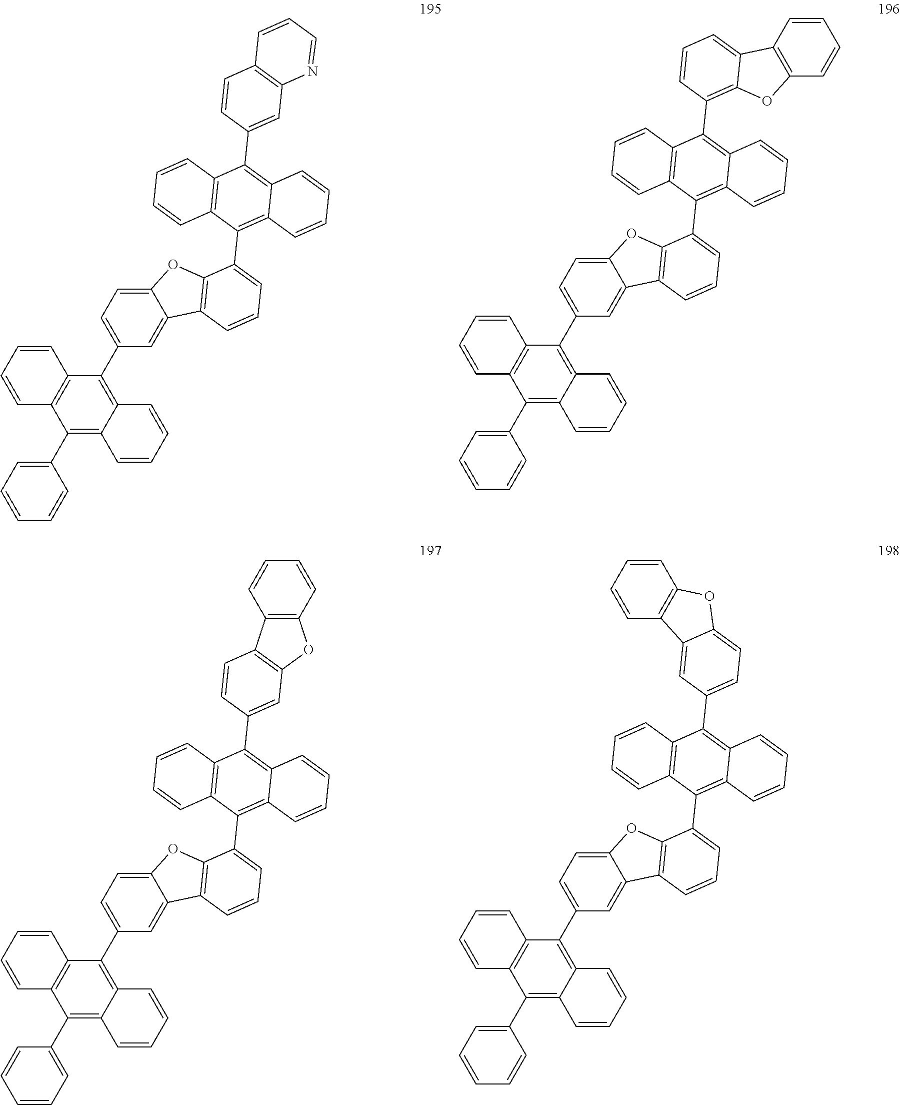 [SMILES: c1ccc(-c2c3ccccc3c(-c3ccc4oc5c(-c6c7ccccc7c(-c7ccc8cccnc8c7)c7ccccc67)cccc5c4c3)c3ccccc23)cc1 ; c1ccc(-c2c3ccccc3c(-c3ccc4oc5c(-c6c7ccccc7c(-c7cccc8c7oc7ccccc78)c7ccccc67)cccc5c4c3)c3ccccc23)cc1 ; c1ccc(-c2c3ccccc3c(-c3ccc4oc5c(-c6c7ccccc7c(-c7ccc8c(c7)oc7ccccc78)c7ccccc67)cccc5c4c3)c3ccccc23)cc1 ; c1ccc(-c2c3ccccc3c(-c3ccc4oc5c(-c6c7ccccc7c(-c7ccc8oc9ccccc9c8c7)c7ccccc67)cccc5c4c3)c3ccccc23)cc1]

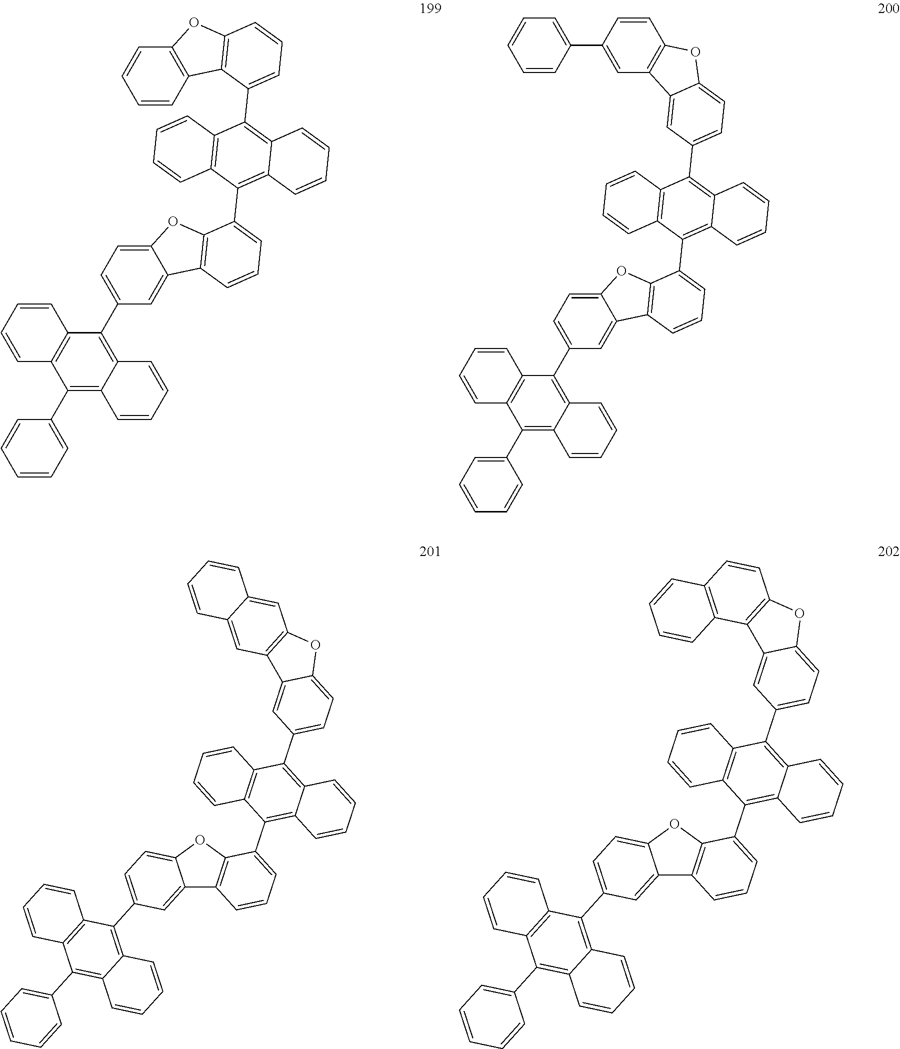 [SMILES: c1ccc(-c2c3ccccc3c(-c3ccc4oc5c(-c6c7ccccc7c(-c7cccc8oc9ccccc9c78)c7ccccc67)cccc5c4c3)c3ccccc23)cc1 ; c1ccc(-c2ccc3oc4ccc(-c5c6ccccc6c(-c6cccc7c6oc6ccc(-c8c9ccccc9c(-c9ccccc9)c9ccccc89)cc67)c6ccccc56)cc4c3c2)cc1 ; c1ccc(-c2c3ccccc3c(-c3ccc4oc5c(-c6c7ccccc7c(-c7ccc8oc9cc%10ccccc%10cc9c8c7)c7ccccc67)cccc5c4c3)c3ccccc23)cc1 ; c1ccc(-c2c3ccccc3c(-c3ccc4oc5c(-c6c7ccccc7c(-c7ccc8oc9ccc%10ccccc%10c9c8c7)c7ccccc67)cccc5c4c3)c3ccccc23)cc1]

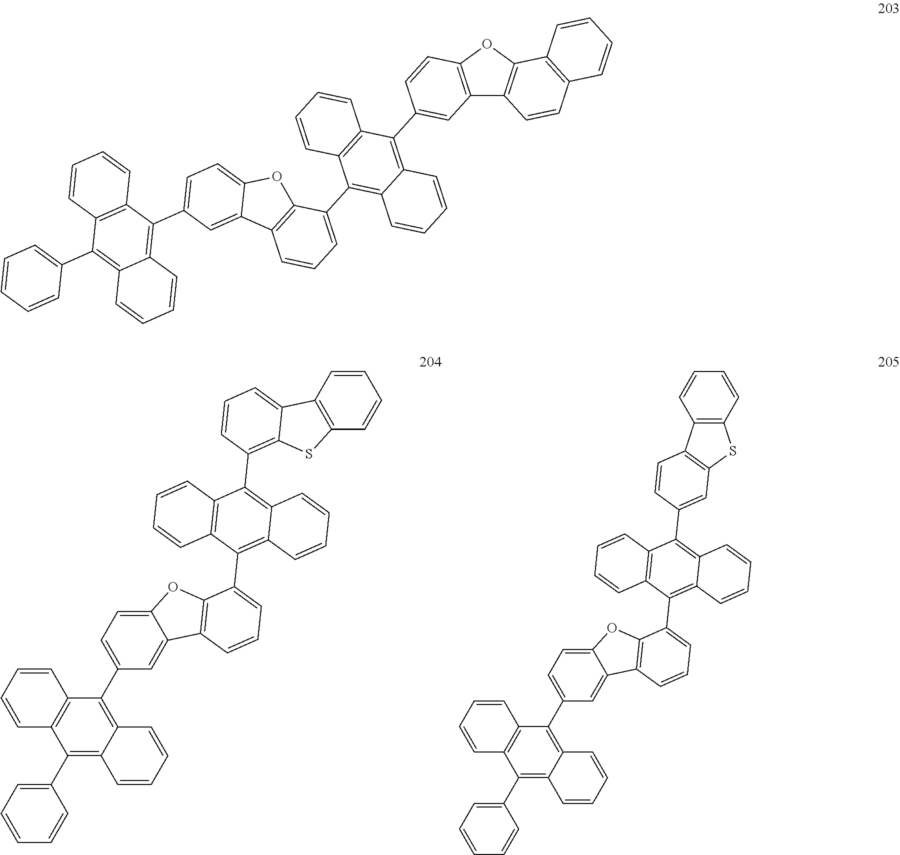 [SMILES: c1ccc(-c2c3ccccc3c(-c3ccc4oc5c(-c6c7ccccc7c(-c7ccc8oc9c%10ccccc%10ccc9c8c7)c7ccccc67)cccc5c4c3)c3ccccc23)cc1 ; c1ccc(-c2c3ccccc3c(-c3ccc4oc5c(-c6c7ccccc7c(-c7cccc8c7sc7ccccc78)c7ccccc67)cccc5c4c3)c3ccccc23)cc1 ; c1ccc(-c2c3ccccc3c(-c3ccc4oc5c(-c6c7ccccc7c(-c7ccc8c(c7)sc7ccccc78)c7ccccc67)cccc5c4c3)c3ccccc23)cc1]

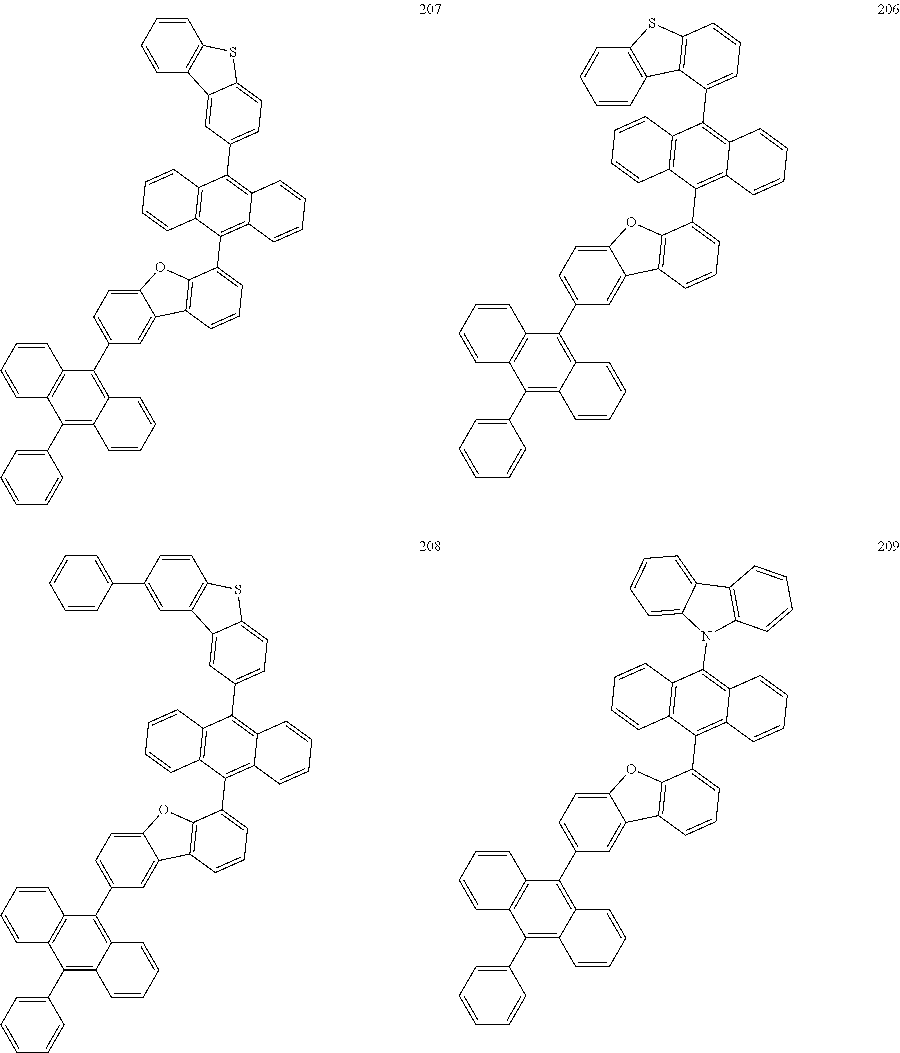 [SMILES: c1ccc(-c2c3ccccc3c(-c3ccc4oc5c(-c6c7ccccc7c(-c7ccc8sc9ccccc9c8c7)c7ccccc67)cccc5c4c3)c3ccccc23)cc1 ; c1ccc(-c2c3ccccc3c(-c3ccc4oc5c(-c6c7ccccc7c(-c7cccc8sc9ccccc9c78)c7ccccc67)cccc5c4c3)c3ccccc23)cc1 ; c1ccc(-c2ccc3sc4ccc(-c5c6ccccc6c(-c6cccc7c6oc6ccc(-c8c9ccccc9c(-c9ccccc9)c9ccccc89)cc67)c6ccccc56)cc4c3c2)cc1 ; c1ccc(-c2c3ccccc3c(-c3ccc4oc5c(-c6c7ccccc7c(-n7c8ccccc8c8ccccc87)c7ccccc67)cccc5c4c3)c3ccccc23)cc1]

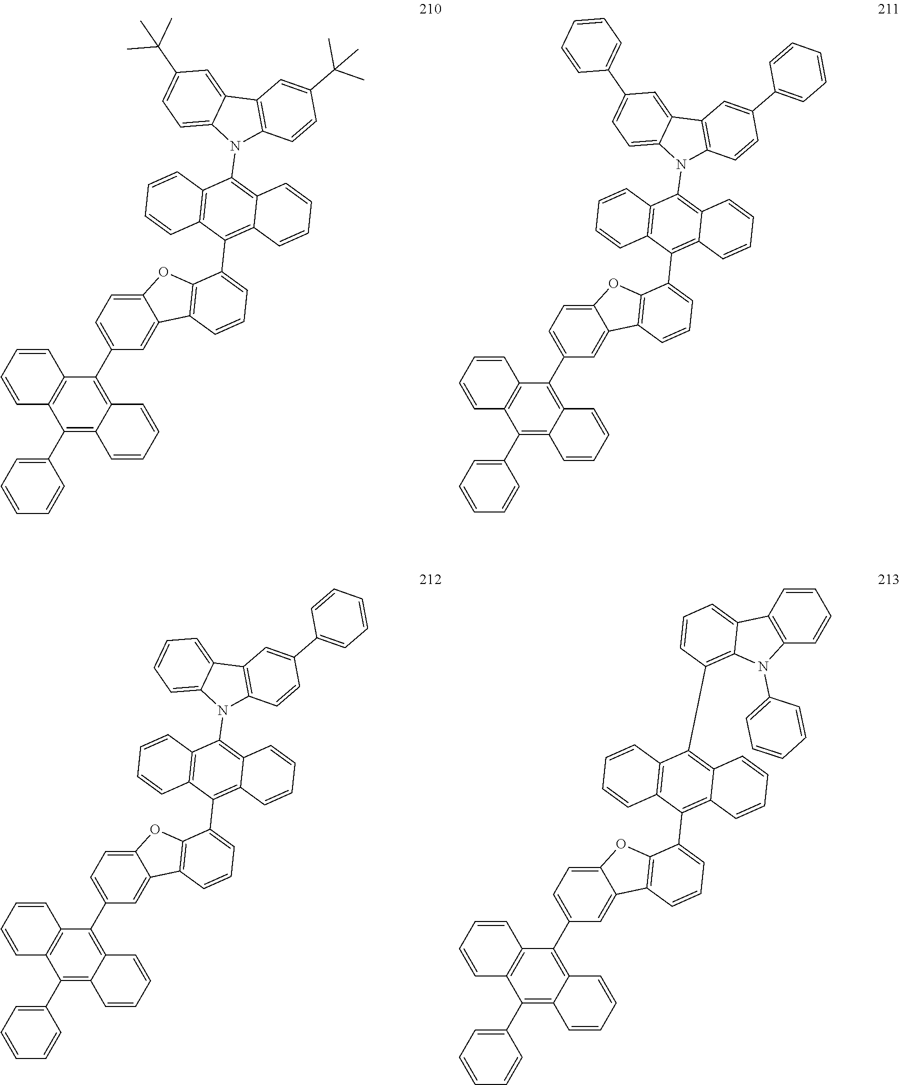 [SMILES: CC(C)(C)c1ccc2c(c1)c1cc(C(C)(C)C)ccc1n2-c1c2ccccc2c(-c2cccc3c2oc2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3cccc4c3oc3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3cccc4c3oc3ccc(-c5c6ccccc6c(-c6ccccc6)c6ccccc56)cc34)c3ccccc23)cc1 ; c1ccc(-c2c3ccccc3c(-c3ccc4oc5c(-c6c7ccccc7c(-c7cccc8c9ccccc9n(-c9ccccc9)c78)c7ccccc67)cccc5c4c3)c3ccccc23)cc1]

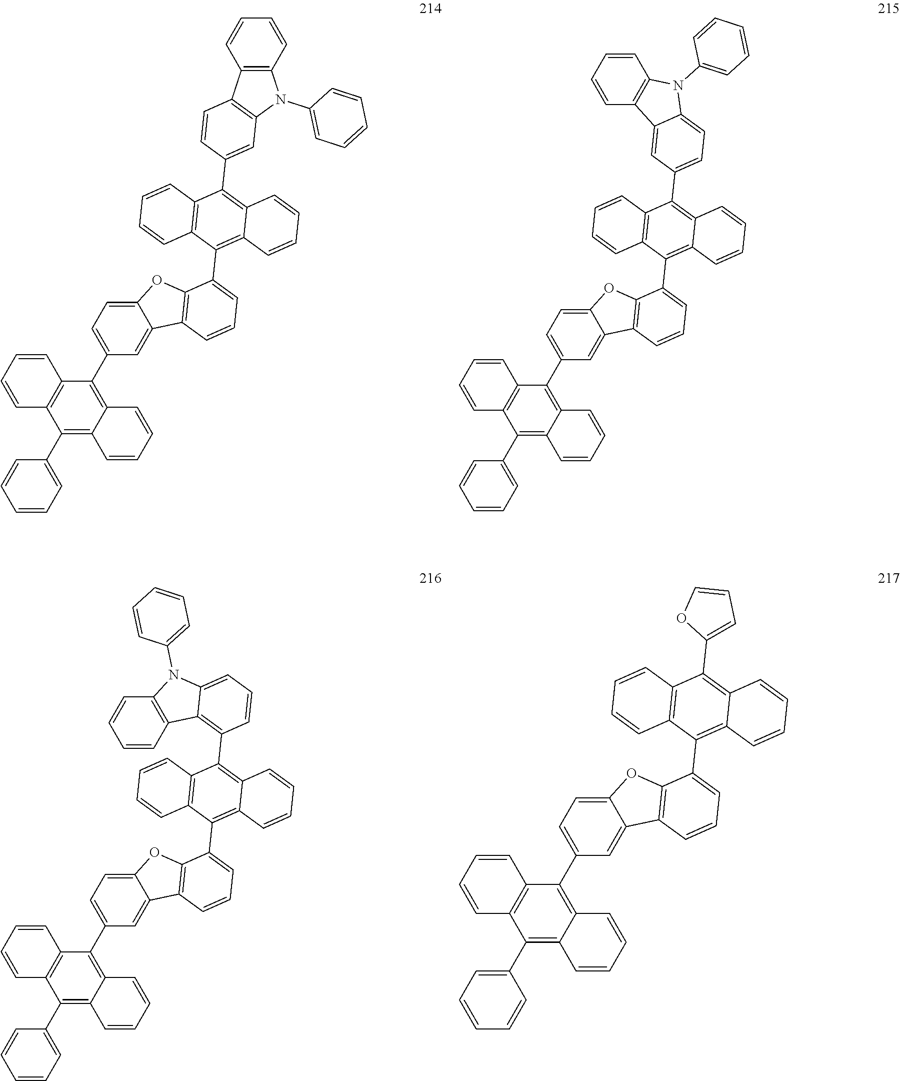 [SMILES: c1ccc(-c2c3ccccc3c(-c3ccc4oc5c(-c6c7ccccc7c(-c7ccc8c9ccccc9n(-c9ccccc9)c8c7)c7ccccc67)cccc5c4c3)c3ccccc23)cc1 ; c1ccc(-c2c3ccccc3c(-c3ccc4oc5c(-c6c7ccccc7c(-c7ccc8c(c7)c7ccccc7n8-c7ccccc7)c7ccccc67)cccc5c4c3)c3ccccc23)cc1 ; c1ccc(-c2c3ccccc3c(-c3ccc4oc5c(-c6c7ccccc7c(-c7cccc8c7c7ccccc7n8-c7ccccc7)c7ccccc67)cccc5c4c3)c3ccccc23)cc1 ; c1ccc(-c2c3ccccc3c(-c3ccc4oc5c(-c6c7ccccc7c(-c7ccco7)c7ccccc67)cccc5c4c3)c3ccccc23)cc1]

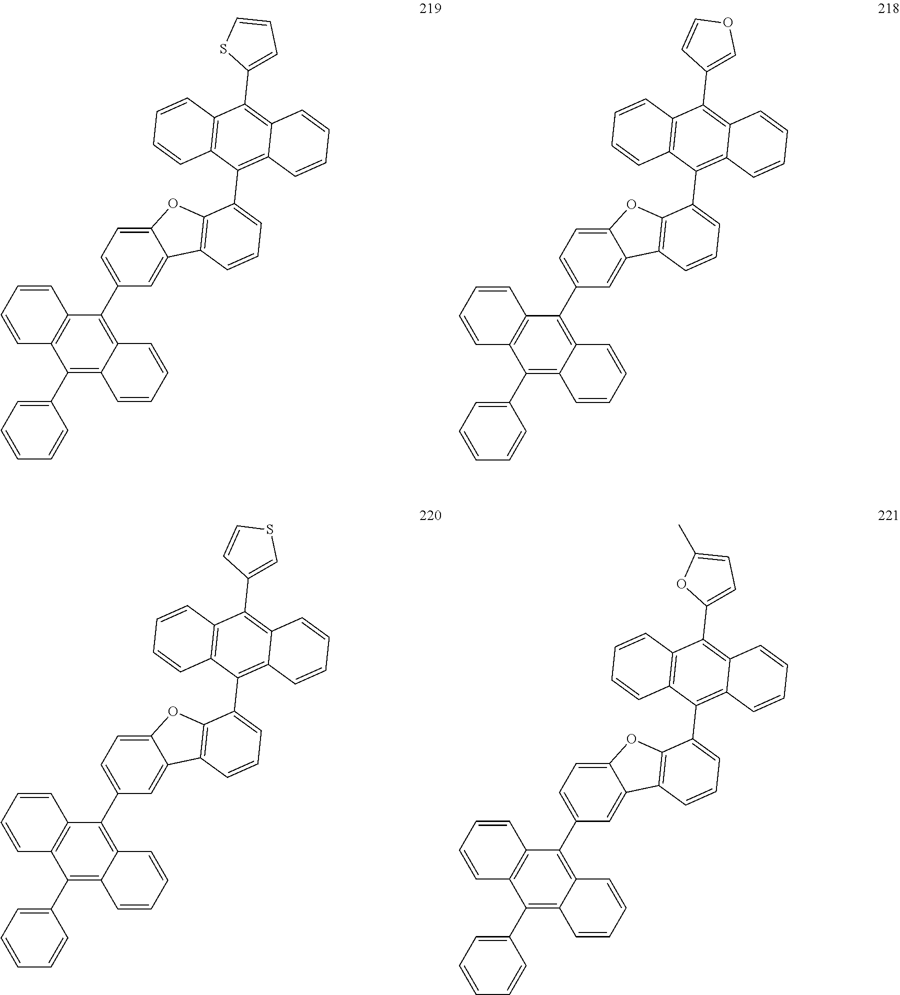 [SMILES: c1ccc(-c2c3ccccc3c(-c3ccc4oc5c(-c6c7ccccc7c(-c7cccs7)c7ccccc67)cccc5c4c3)c3ccccc23)cc1 ; c1ccc(-c2c3ccccc3c(-c3ccc4oc5c(-c6c7ccccc7c(-c7ccoc7)c7ccccc67)cccc5c4c3)c3ccccc23)cc1 ; c1ccc(-c2c3ccccc3c(-c3ccc4oc5c(-c6c7ccccc7c(-c7ccsc7)c7ccccc67)cccc5c4c3)c3ccccc23)cc1 ; Cc1ccc(-c2c3ccccc3c(-c3cccc4c3oc3ccc(-c5c6ccccc6c(-c6ccccc6)c6ccccc56)cc34)c3ccccc23)o1]

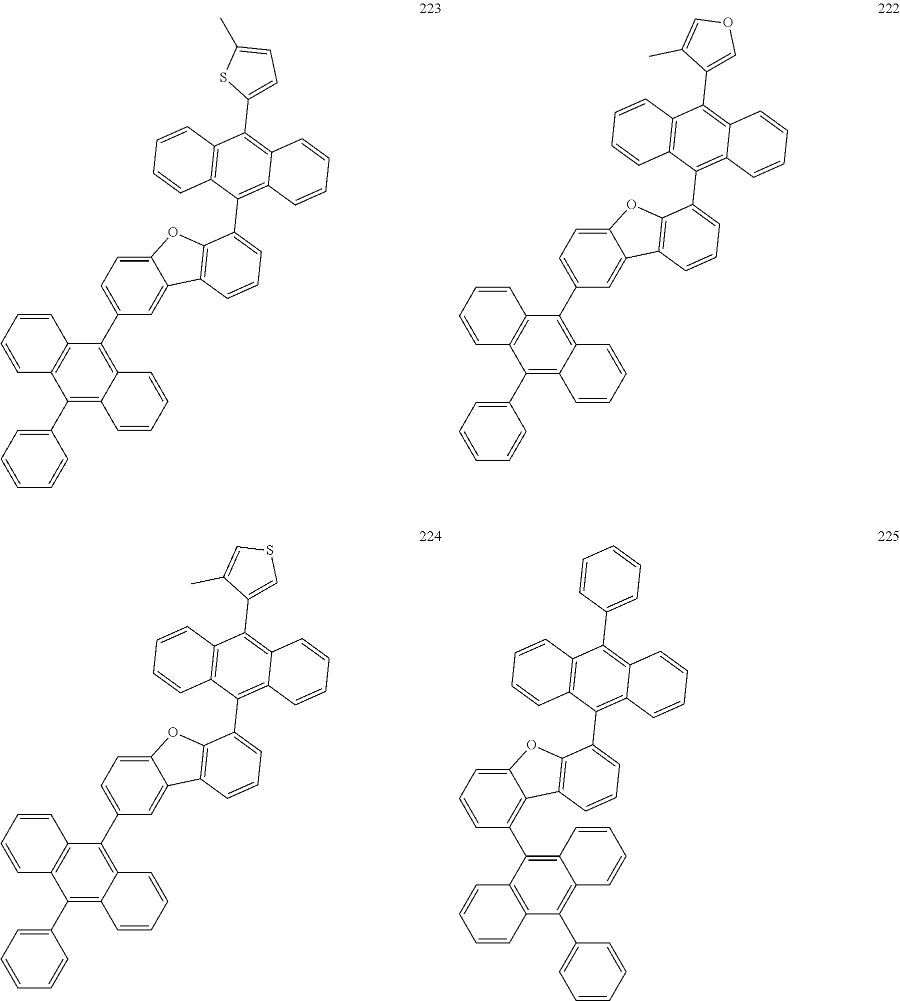 [SMILES: Cc1ccc(-c2c3ccccc3c(-c3cccc4c3oc3ccc(-c5c6ccccc6c(-c6ccccc6)c6ccccc56)cc34)c3ccccc23)s1 ; Cc1cocc1-c1c2ccccc2c(-c2cccc3c2oc2ccc(-c4c5ccccc5c(-c5ccccc5)c5ccccc45)cc23)c2ccccc12 ; Cc1cscc1-c1c2ccccc2c(-c2cccc3c2oc2ccc(-c4c5ccccc5c(-c5ccccc5)c5ccccc45)cc23)c2ccccc12 ; c1ccc(-c2c3ccccc3c(-c3cccc4c3oc3cccc(-c5c6ccccc6c(-c6ccccc6)c6ccccc56)c34)c3ccccc23)cc1]

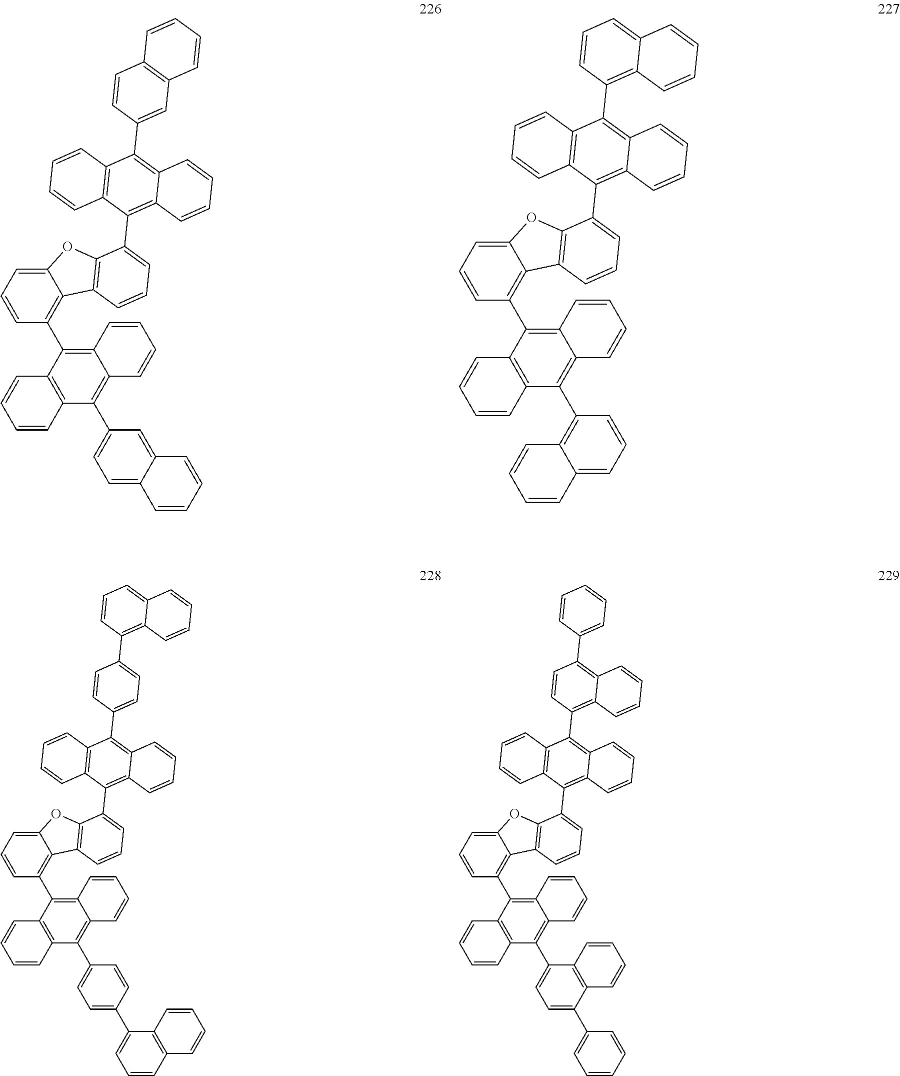 [SMILES: c1ccc2cc(-c3c4ccccc4c(-c4cccc5c4oc4cccc(-c6c7ccccc7c(-c7ccc8ccccc8c7)c7ccccc67)c45)c4ccccc34)ccc2c1 ; c1ccc2c(-c3c4ccccc4c(-c4cccc5c4oc4cccc(-c6c7ccccc7c(-c7cccc8ccccc78)c7ccccc67)c45)c4ccccc34)cccc2c1 ; c1ccc2c(-c3ccc(-c4c5ccccc5c(-c5cccc6c5oc5cccc(-c7c8ccccc8c(-c8ccc(-c9cccc%10ccccc9%10)cc8)c8ccccc78)c56)c5ccccc45)cc3)cccc2c1 ; c1ccc(-c2ccc(-c3c4ccccc4c(-c4cccc5c4oc4cccc(-c6c7ccccc7c(-c7ccc(-c8ccccc8)c8ccccc78)c7ccccc67)c45)c4ccccc34)c3ccccc23)cc1]

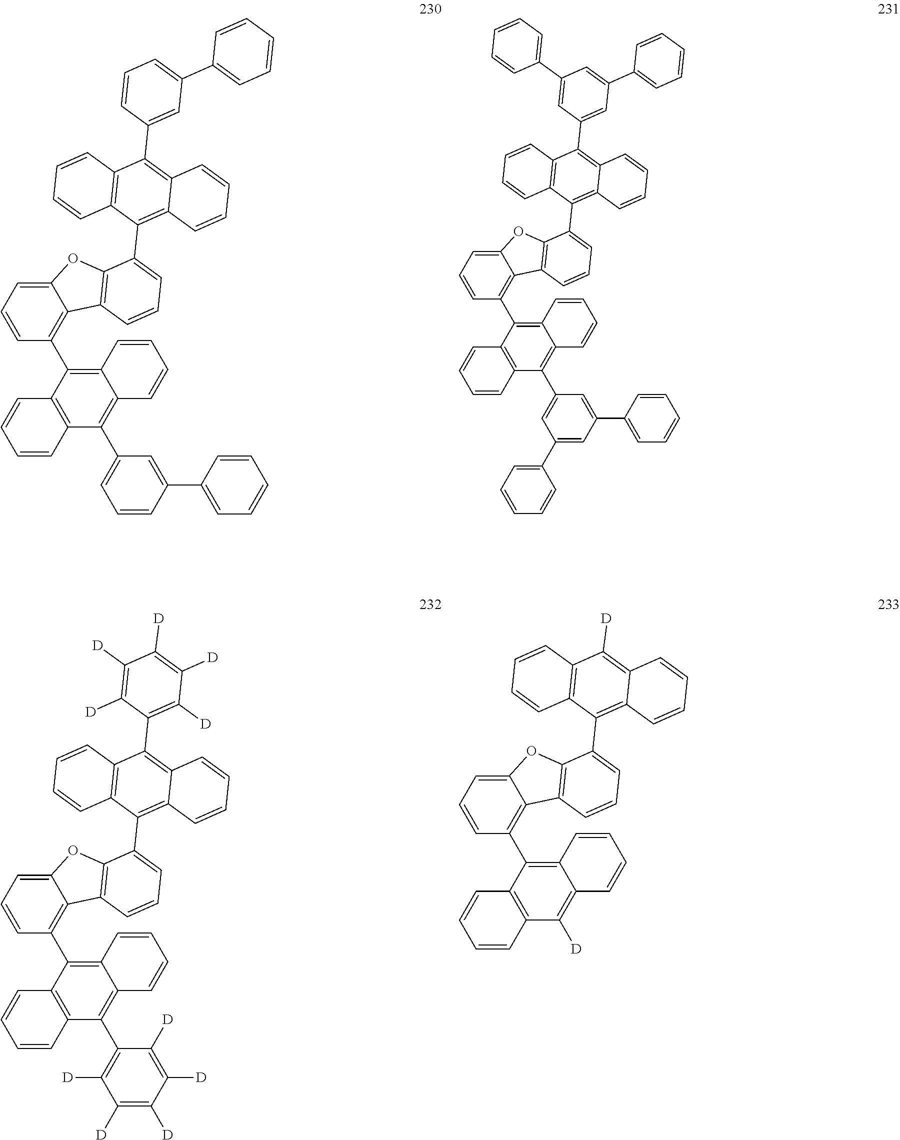 [SMILES: c1ccc(-c2cccc(-c3c4ccccc4c(-c4cccc5c4oc4cccc(-c6c7ccccc7c(-c7cccc(-c8ccccc8)c7)c7ccccc67)c45)c4ccccc34)c2)cc1 ; c1ccc(-c2cc(-c3ccccc3)cc(-c3c4ccccc4c(-c4cccc5c4oc4cccc(-c6c7ccccc7c(-c7cc(-c8ccccc8)cc(-c8ccccc8)c7)c7ccccc67)c45)c4ccccc34)c2)cc1 ; [2H]c1c([2H])c([2H])c(-c2c3ccccc3c(-c3cccc4c3oc3cccc(-c5c6ccccc6c(-c6c([2H])c([2H])c([2H])c([2H])c6[2H])c6ccccc56)c34)c3ccccc23)c([2H])c1[2H] ; [2H]c1c2ccccc2c(-c2cccc3c2oc2cccc(-c4c5ccccc5c([2H])c5ccccc45)c23)c2ccccc12]

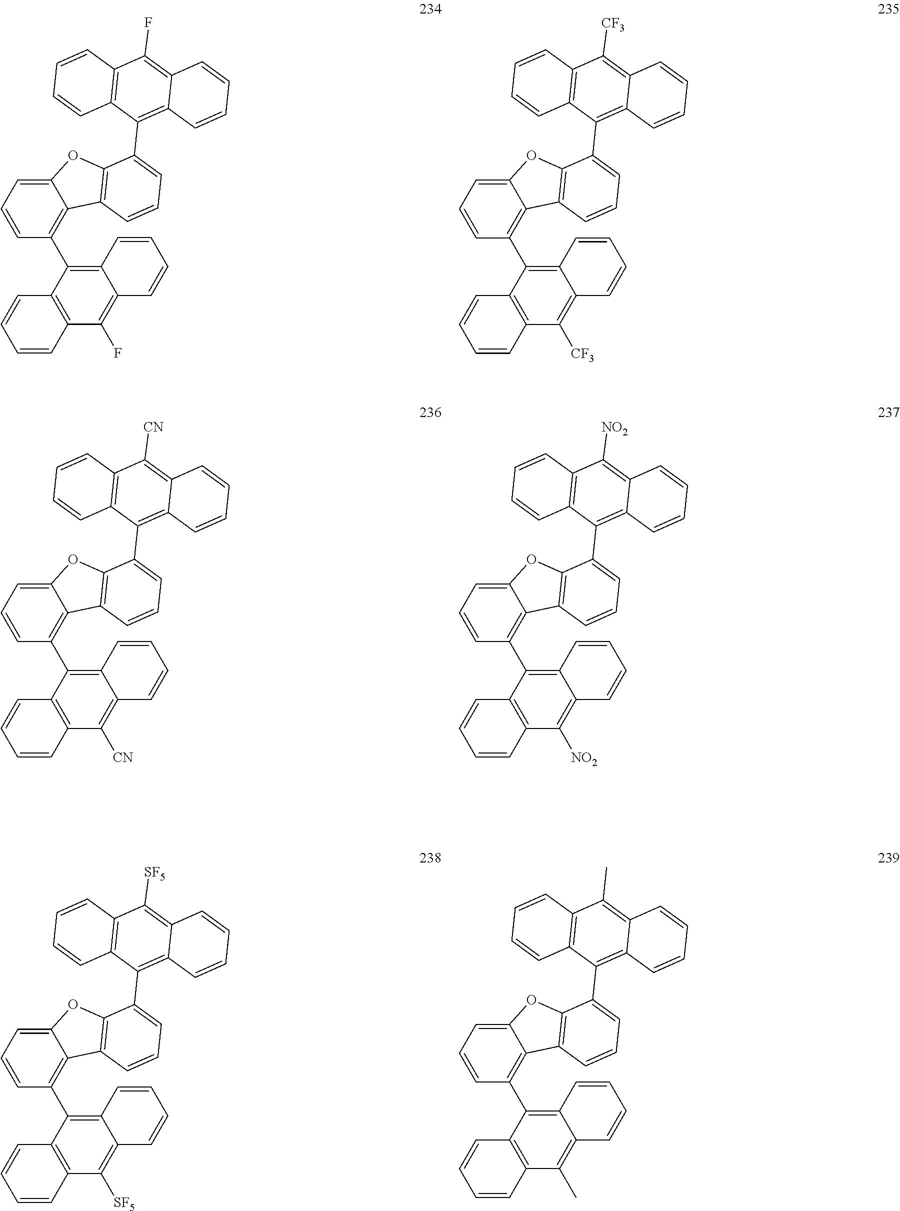

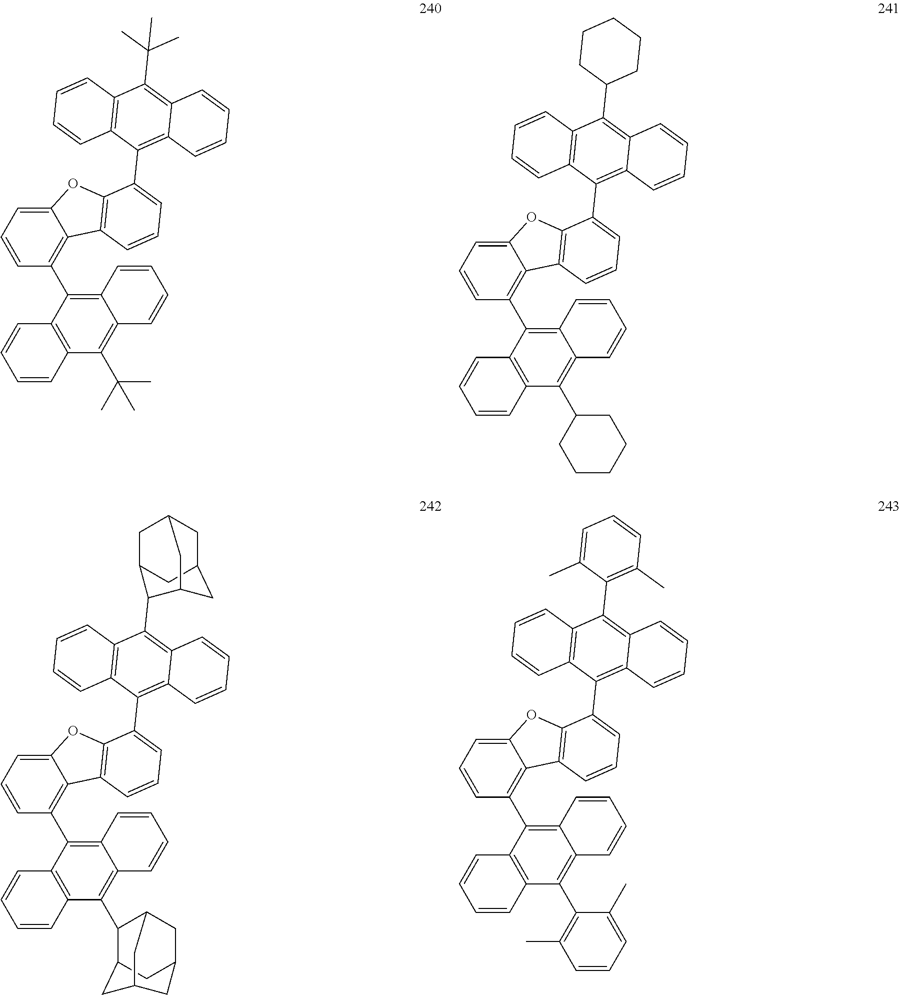

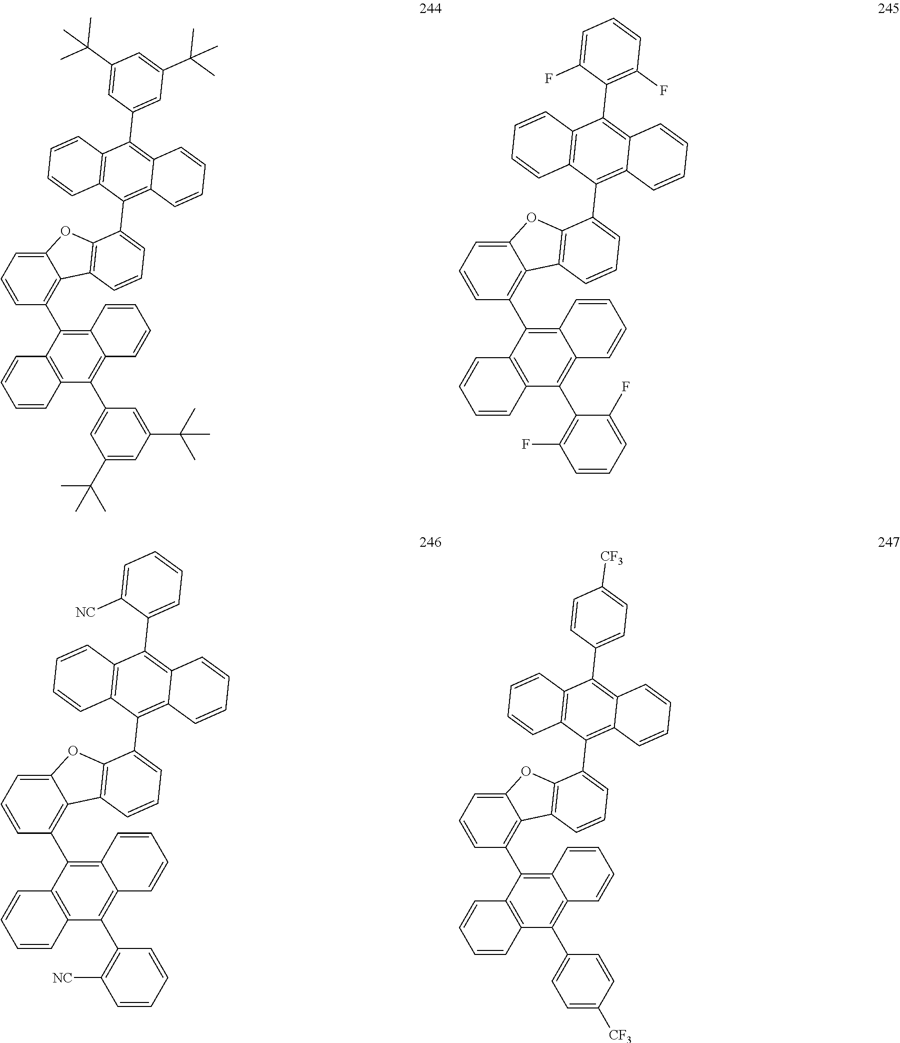 [SMILES: CC(C)(C)c1cc(-c2c3ccccc3c(-c3cccc4c3oc3cccc(-c5c6ccccc6c(-c6cc(C(C)(C)C)cc(C(C)(C)C)c6)c6ccccc56)c34)c3ccccc23)cc(C(C)(C)C)c1 ; Fc1cccc(F)c1-c1c2ccccc2c(-c2cccc3c2oc2cccc(-c4c5ccccc5c(-c5c(F)cccc5F)c5ccccc45)c23)c2ccccc12 ; N#Cc1ccccc1-c1c2ccccc2c(-c2cccc3c2oc2cccc(-c4c5ccccc5c(-c5ccccc5C#N)c5ccccc45)c23)c2ccccc12 ; FC(F)(F)c1ccc(-c2c3ccccc3c(-c3cccc4c3oc3cccc(-c5c6ccccc6c(-c6ccc(C(F)(F)F)cc6)c6ccccc56)c34)c3ccccc23)cc1]

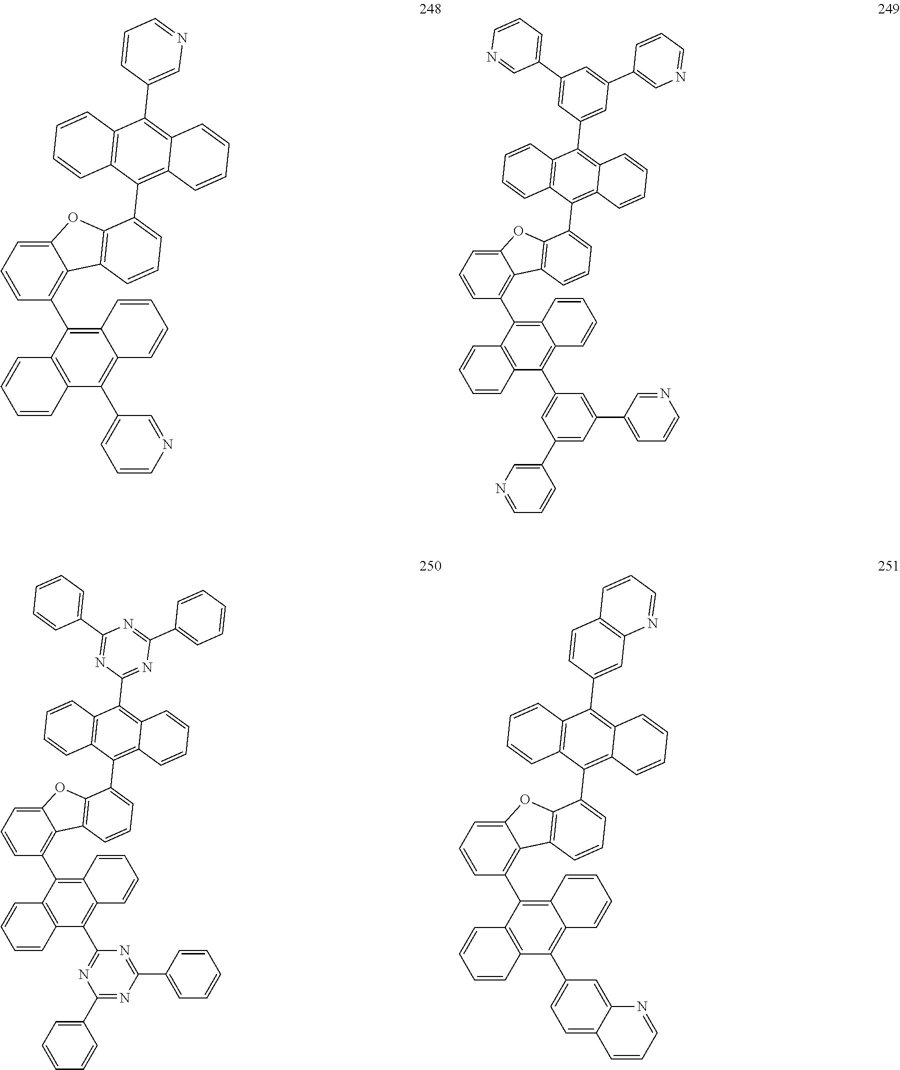 [SMILES: c1cncc(-c2c3ccccc3c(-c3cccc4c3oc3cccc(-c5c6ccccc6c(-c6cccnc6)c6ccccc56)c34)c3ccccc23)c1 ; c1cncc(-c2cc(-c3cccnc3)cc(-c3c4ccccc4c(-c4cccc5c4oc4cccc(-c6c7ccccc7c(-c7cc(-c8cccnc8)cc(-c8cccnc8)c7)c7ccccc67)c45)c4ccccc34)c2)c1 ; c1ccc(-c2nc(-c3ccccc3)nc(-c3c4ccccc4c(-c4cccc5c4oc4cccc(-c6c7ccccc7c(-c7nc(-c8ccccc8)nc(-c8ccccc8)n7)c7ccccc67)c45)c4ccccc34)n2)cc1 ; c1cnc2cc(-c3c4ccccc4c(-c4cccc5c4oc4cccc(-c6c7ccccc7c(-c7ccc8cccnc8c7)c7ccccc67)c45)c4ccccc34)ccc2c1]

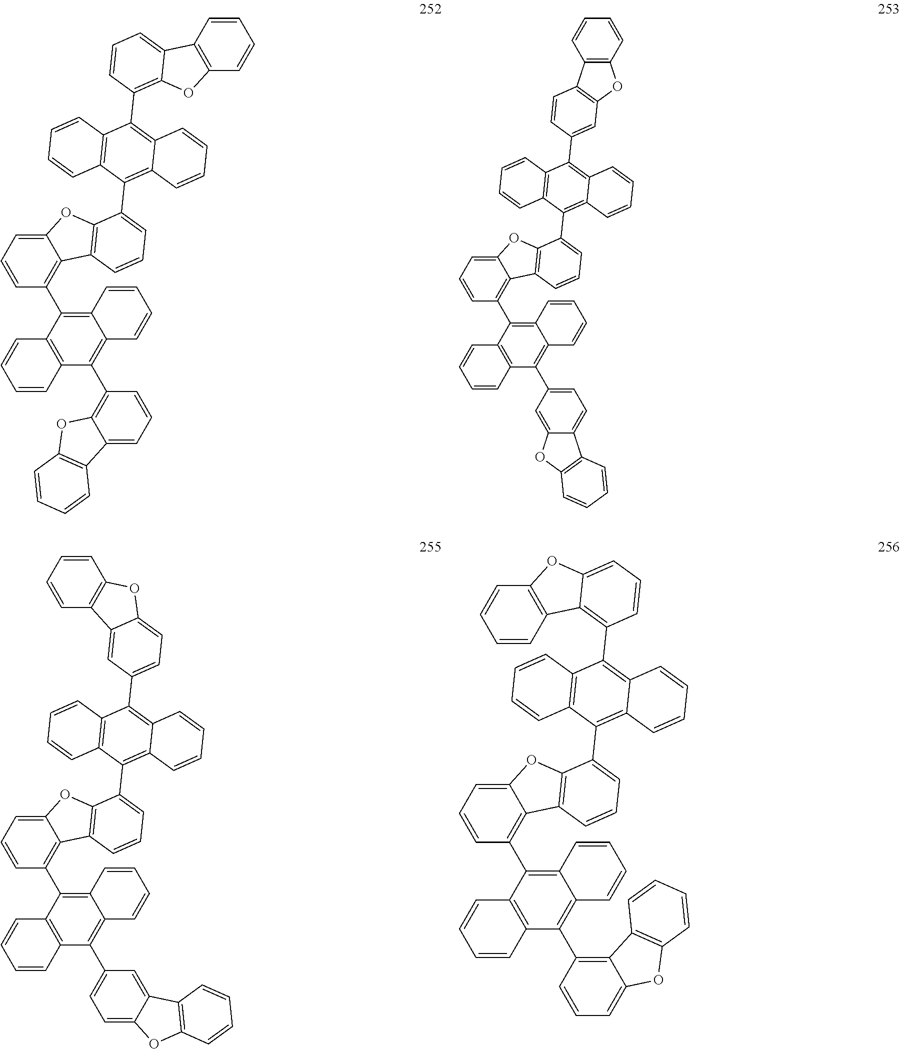 [SMILES: c1ccc2c(c1)oc1c(-c3c4ccccc4c(-c4cccc5c4oc4cccc(-c6c7ccccc7c(-c7cccc8c7oc7ccccc78)c7ccccc67)c45)c4ccccc34)cccc12 ; c1ccc2c(c1)oc1cc(-c3c4ccccc4c(-c4cccc5c4oc4cccc(-c6c7ccccc7c(-c7ccc8c(c7)oc7ccccc78)c7ccccc67)c45)c4ccccc34)ccc12 ; c1ccc2c(c1)oc1ccc(-c3c4ccccc4c(-c4cccc5c4oc4cccc(-c6c7ccccc7c(-c7ccc8oc9ccccc9c8c7)c7ccccc67)c45)c4ccccc34)cc12 ; c1ccc2c(c1)oc1cccc(-c3c4ccccc4c(-c4cccc5c4oc4cccc(-c6c7ccccc7c(-c7cccc8oc9ccccc9c78)c7ccccc67)c45)c4ccccc34)c12]

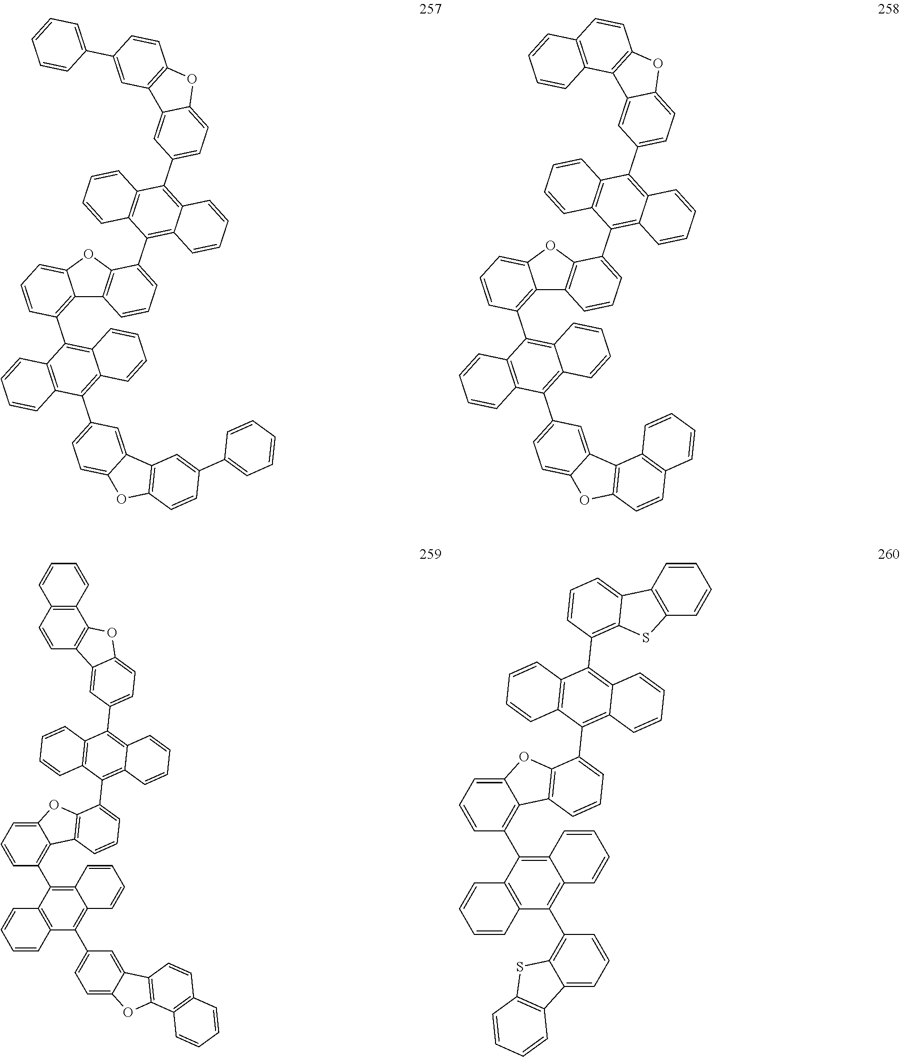

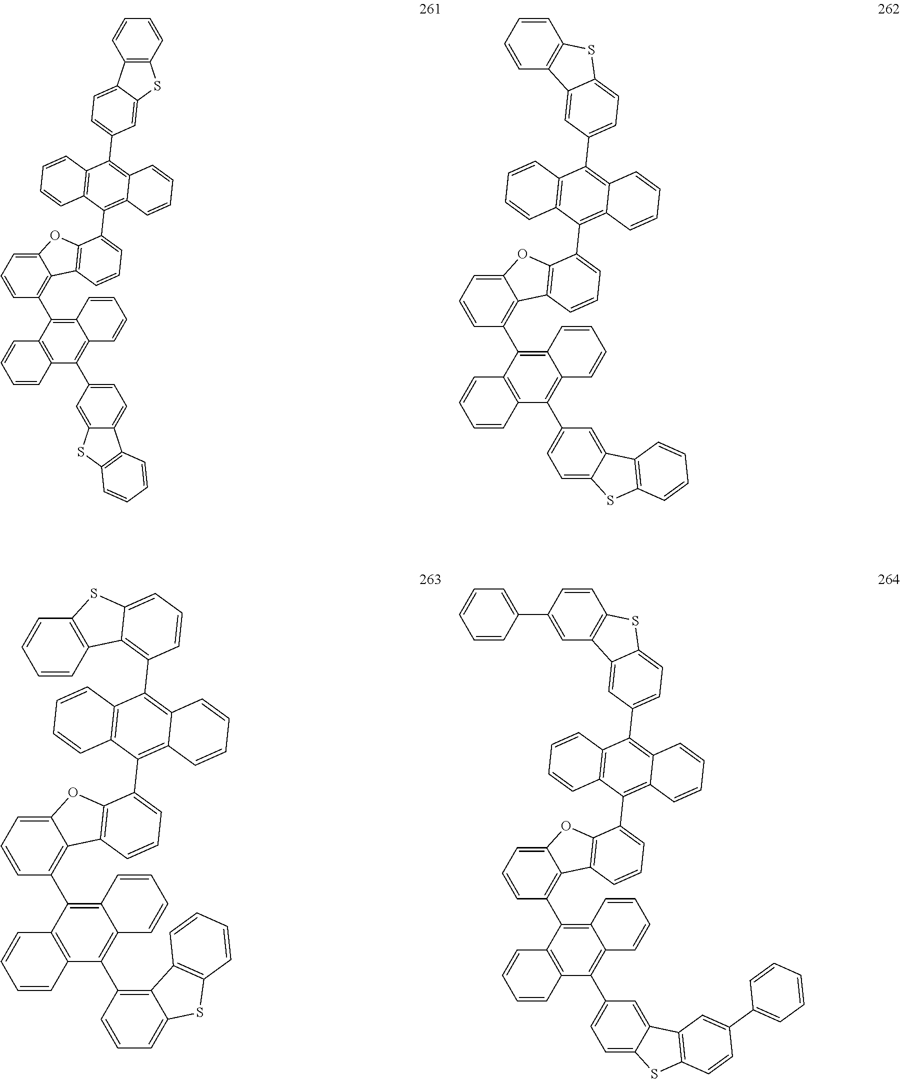 [SMILES: c1cc(-c2c3ccccc3c(-c3ccc4c(c3)sc3ccccc34)c3ccccc23)c2c(c1)oc1c(-c3c4ccccc4c(-c4ccc5c(c4)sc4ccccc45)c4ccccc34)cccc12 ; c1cc(-c2c3ccccc3c(-c3ccc4sc5ccccc5c4c3)c3ccccc23)c2c(c1)oc1c(-c3c4ccccc4c(-c4ccc5sc6ccccc6c5c4)c4ccccc34)cccc12 ; c1cc(-c2c3ccccc3c(-c3cccc4sc5ccccc5c34)c3ccccc23)c2c(c1)oc1c(-c3c4ccccc4c(-c4cccc5sc6ccccc6c45)c4ccccc34)cccc12 ; c1ccc(-c2ccc3sc4ccc(-c5c6ccccc6c(-c6cccc7c6oc6cccc(-c8c9ccccc9c(-c9ccc%10sc%11ccc(-c%12ccccc%12)cc%11c%10c9)c9ccccc89)c67)c6ccccc56)cc4c3c2)cc1]

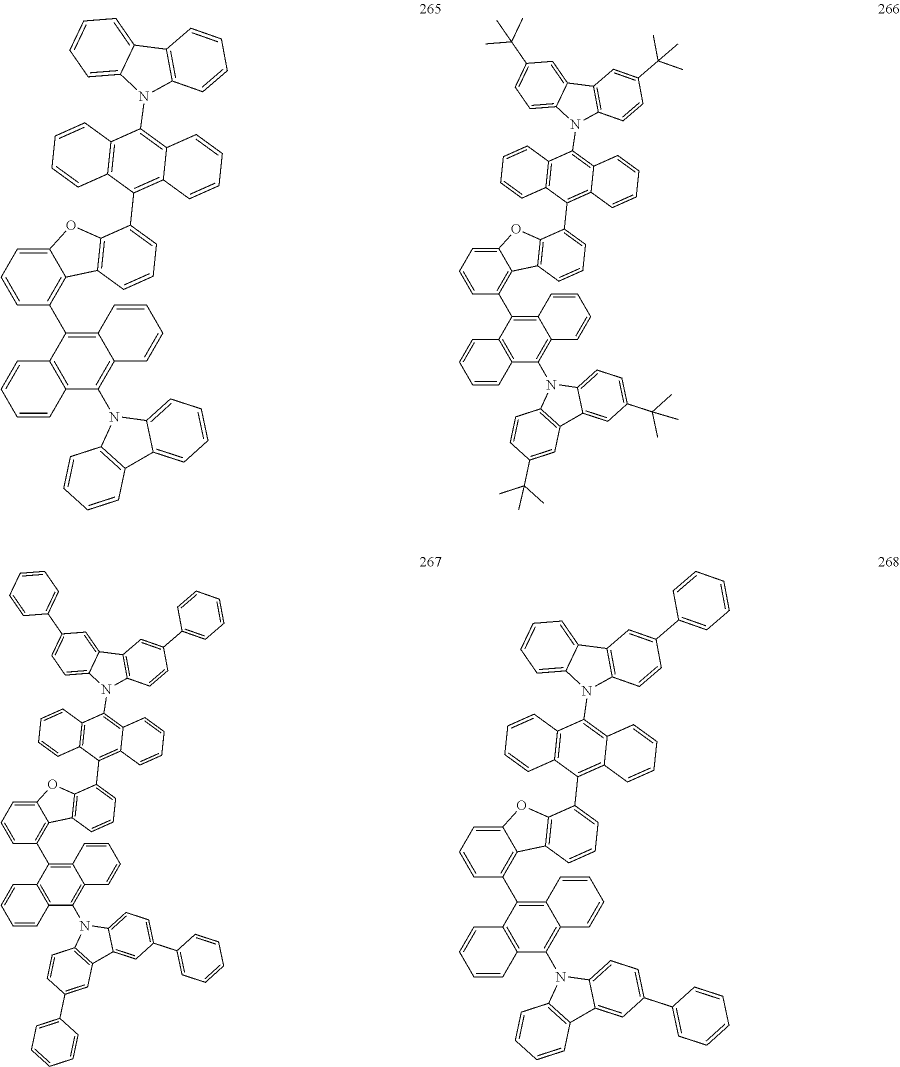

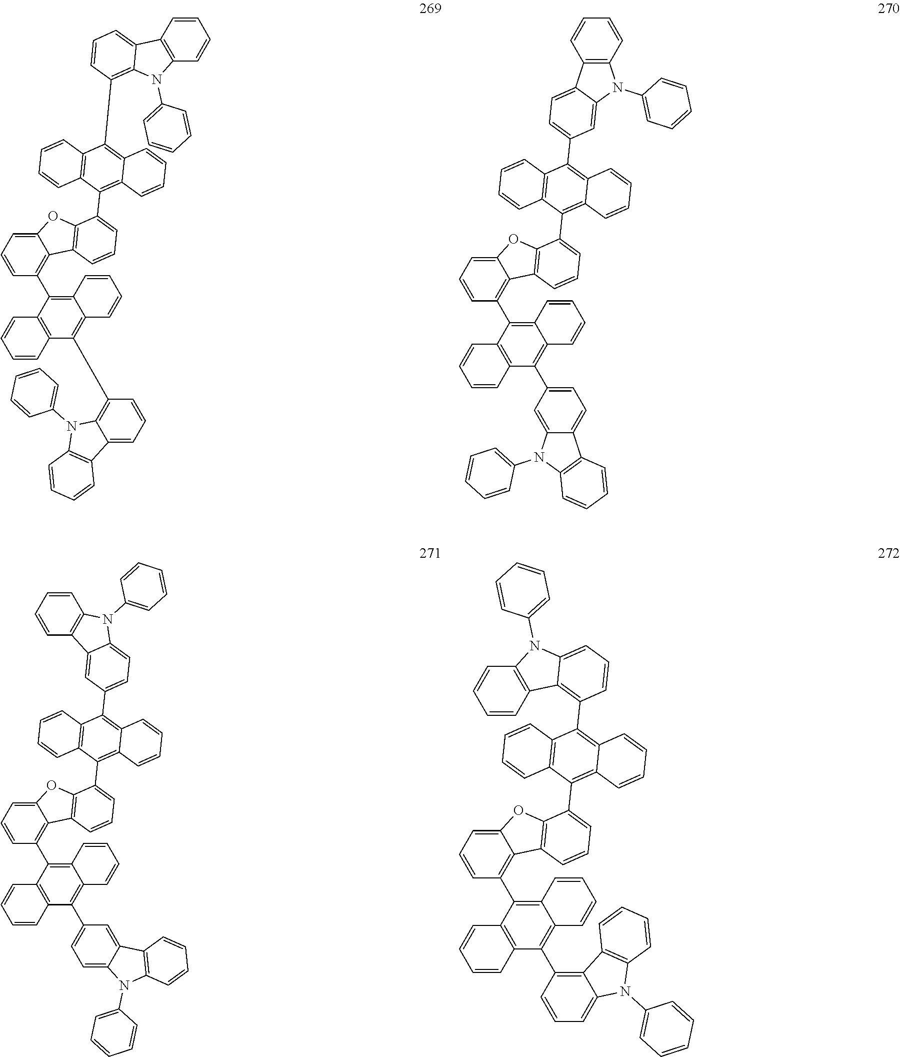

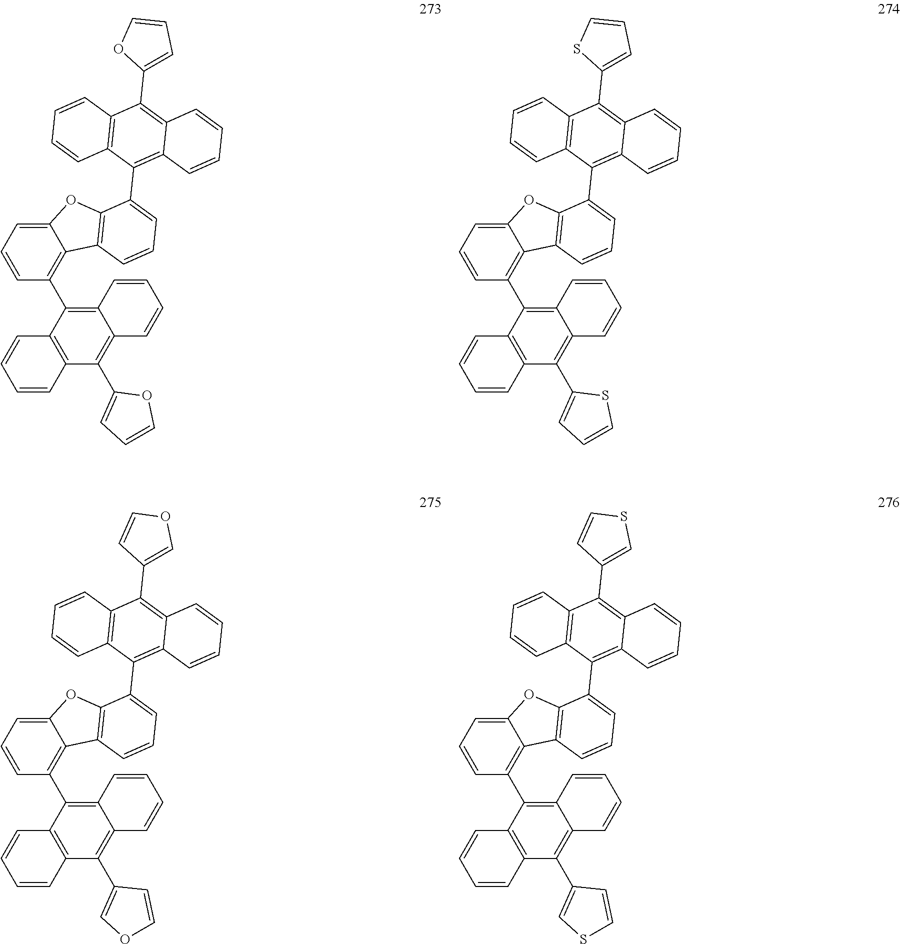 [SMILES: c1coc(-c2c3ccccc3c(-c3cccc4c3oc3cccc(-c5c6ccccc6c(-c6ccco6)c6ccccc56)c34)c3ccccc23)c1 ; c1csc(-c2c3ccccc3c(-c3cccc4c3oc3cccc(-c5c6ccccc6c(-c6cccs6)c6ccccc56)c34)c3ccccc23)c1 ; c1cc(-c2c3ccccc3c(-c3ccoc3)c3ccccc23)c2c(c1)oc1c(-c3c4ccccc4c(-c4ccoc4)c4ccccc34)cccc12 ; c1cc(-c2c3ccccc3c(-c3ccsc3)c3ccccc23)c2c(c1)oc1c(-c3c4ccccc4c(-c4ccsc4)c4ccccc34)cccc12]

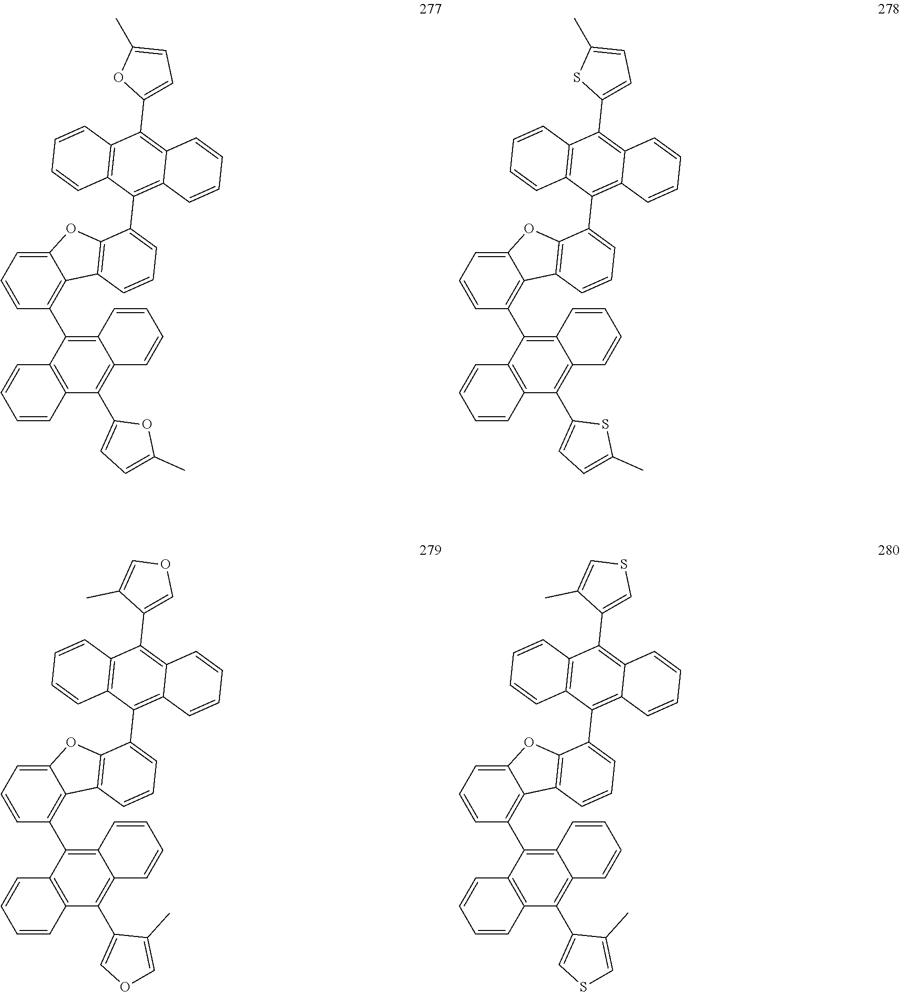 [SMILES: Cc1ccc(-c2c3ccccc3c(-c3cccc4c3oc3cccc(-c5c6ccccc6c(-c6ccc(C)o6)c6ccccc56)c34)c3ccccc23)o1 ; Cc1ccc(-c2c3ccccc3c(-c3cccc4c3oc3cccc(-c5c6ccccc6c(-c6ccc(C)s6)c6ccccc56)c34)c3ccccc23)s1 ; Cc1cocc1-c1c2ccccc2c(-c2cccc3c2oc2cccc(-c4c5ccccc5c(-c5cocc5C)c5ccccc45)c23)c2ccccc12 ; Cc1cscc1-c1c2ccccc2c(-c2cccc3c2oc2cccc(-c4c5ccccc5c(-c5cscc5C)c5ccccc45)c23)c2ccccc12]

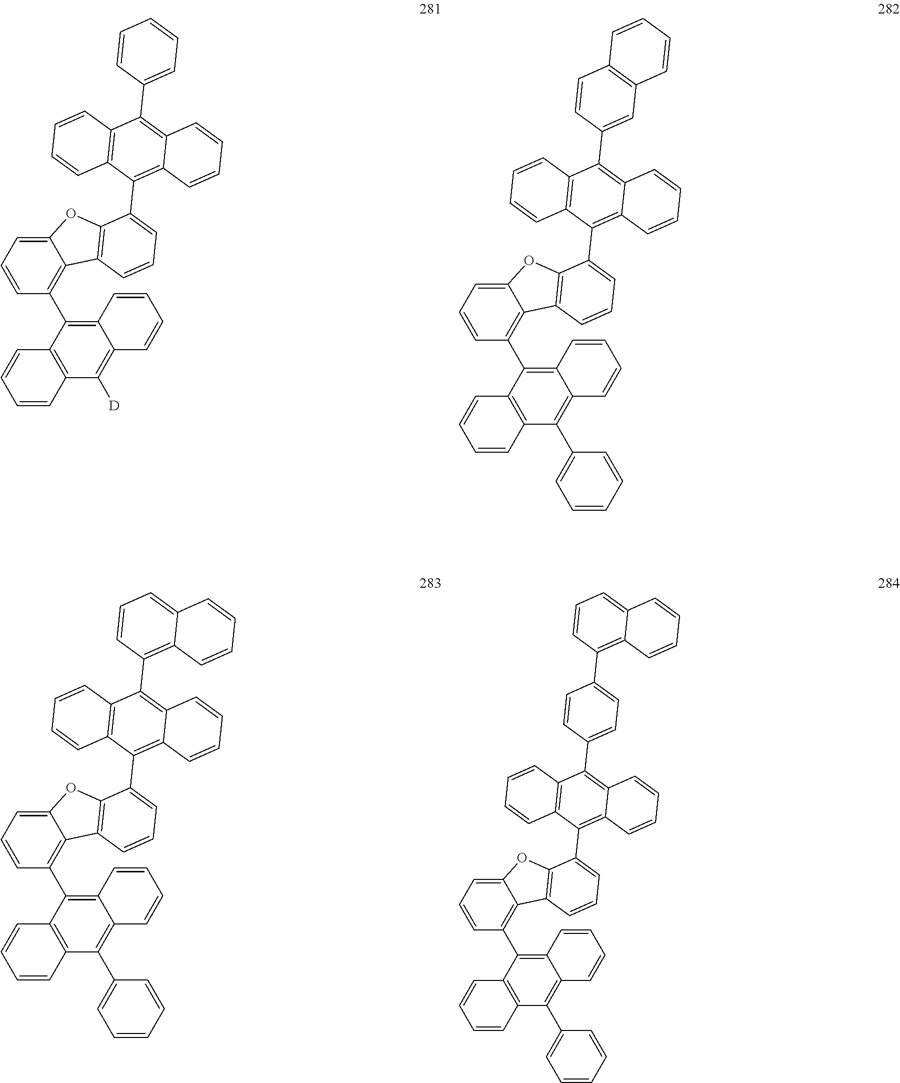 [SMILES: [2H]c1c2ccccc2c(-c2cccc3oc4c(-c5c6ccccc6c(-c6ccccc6)c6ccccc56)cccc4c23)c2ccccc12 ; c1ccc(-c2c3ccccc3c(-c3cccc4oc5c(-c6c7ccccc7c(-c7ccc8ccccc8c7)c7ccccc67)cccc5c34)c3ccccc23)cc1 ; c1ccc(-c2c3ccccc3c(-c3cccc4oc5c(-c6c7ccccc7c(-c7cccc8ccccc78)c7ccccc67)cccc5c34)c3ccccc23)cc1 ; c1ccc(-c2c3ccccc3c(-c3cccc4oc5c(-c6c7ccccc7c(-c7ccc(-c8cccc9ccccc89)cc7)c7ccccc67)cccc5c34)c3ccccc23)cc1]

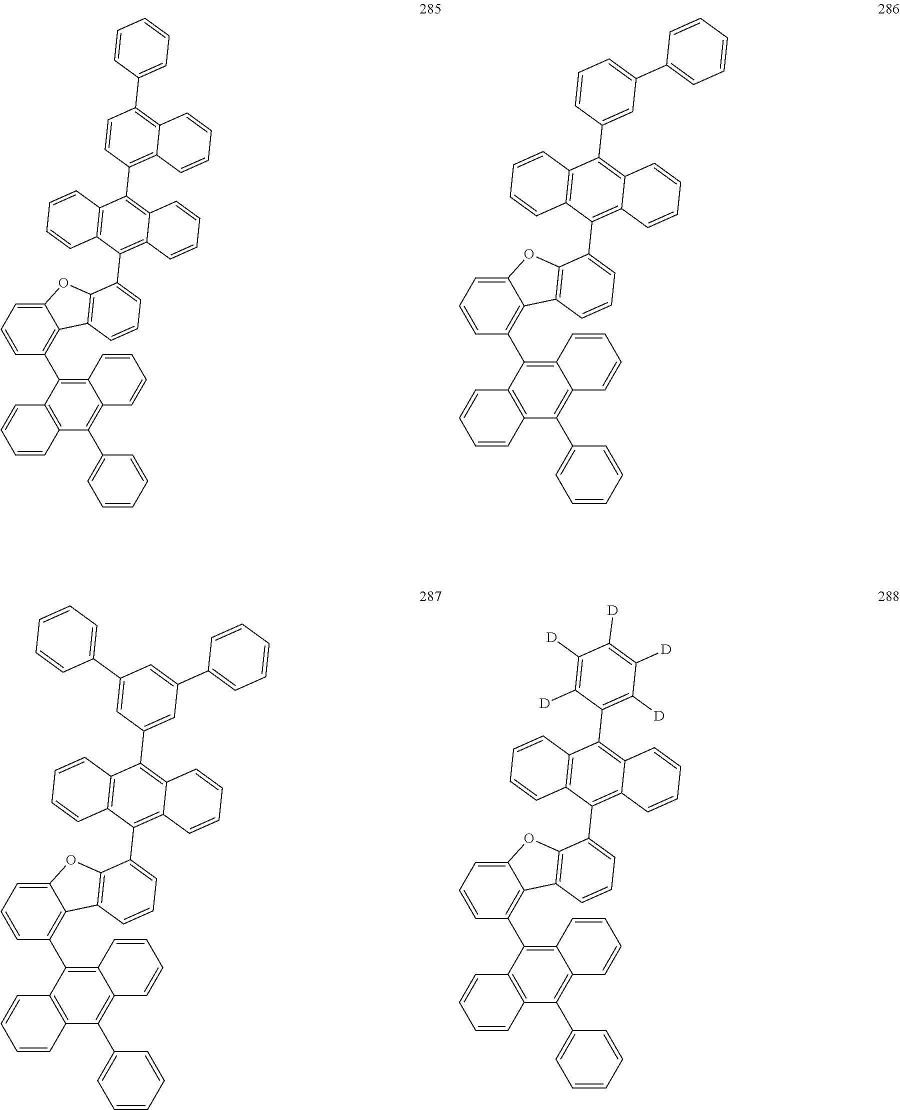 [SMILES: c1ccc(-c2ccc(-c3c4ccccc4c(-c4cccc5c4oc4cccc(-c6c7ccccc7c(-c7ccccc7)c7ccccc67)c45)c4ccccc34)c3ccccc23)cc1 ; c1ccc(-c2cccc(-c3c4ccccc4c(-c4cccc5c4oc4cccc(-c6c7ccccc7c(-c7ccccc7)c7ccccc67)c45)c4ccccc34)c2)cc1 ; c1ccc(-c2cc(-c3ccccc3)cc(-c3c4ccccc4c(-c4cccc5c4oc4cccc(-c6c7ccccc7c(-c7ccccc7)c7ccccc67)c45)c4ccccc34)c2)cc1 ; [2H]c1c([2H])c([2H])c(-c2c3ccccc3c(-c3cccc4c3oc3cccc(-c5c6ccccc6c(-c6ccccc6)c6ccccc56)c34)c3ccccc23)c([2H])c1[2H]]

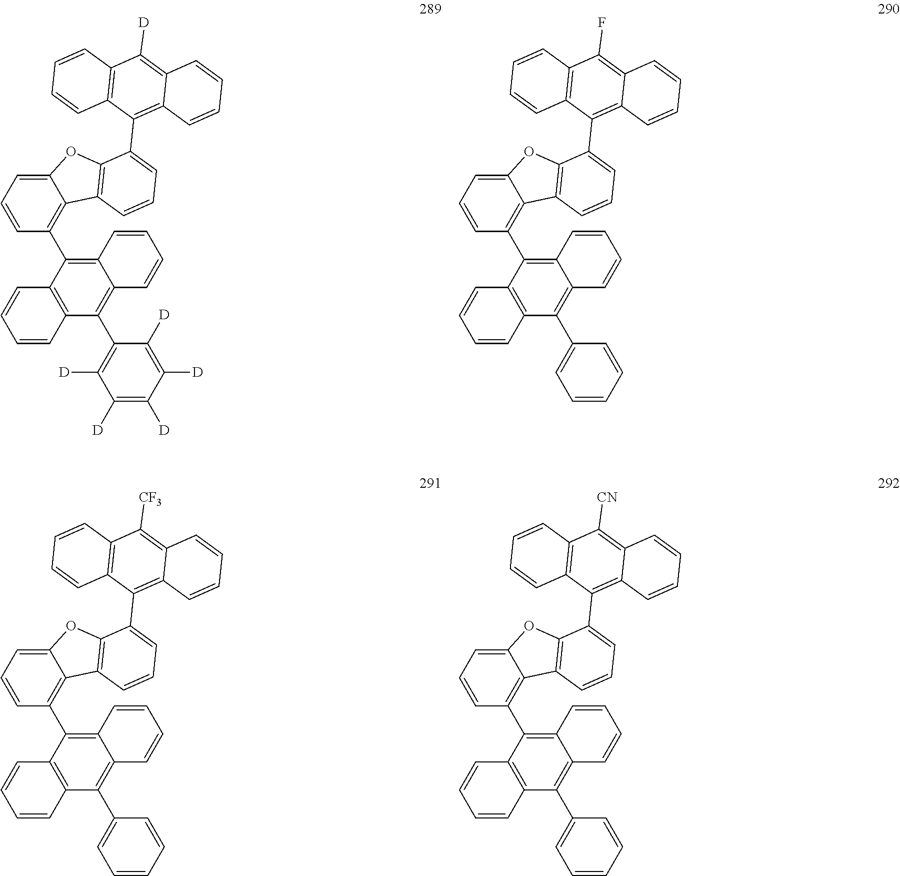 [SMILES: [2H]c1c([2H])c([2H])c(-c2c3ccccc3c(-c3cccc4oc5c(-c6c7ccccc7c([2H])c7ccccc67)cccc5c34)c3ccccc23)c([2H])c1[2H] ; Fc1c2ccccc2c(-c2cccc3c2oc2cccc(-c4c5ccccc5c(-c5ccccc5)c5ccccc45)c23)c2ccccc12 ; FC(F)(F)c1c2ccccc2c(-c2cccc3c2oc2cccc(-c4c5ccccc5c(-c5ccccc5)c5ccccc45)c23)c2ccccc12 ; N#Cc1c2ccccc2c(-c2cccc3c2oc2cccc(-c4c5ccccc5c(-c5ccccc5)c5ccccc45)c23)c2ccccc12]

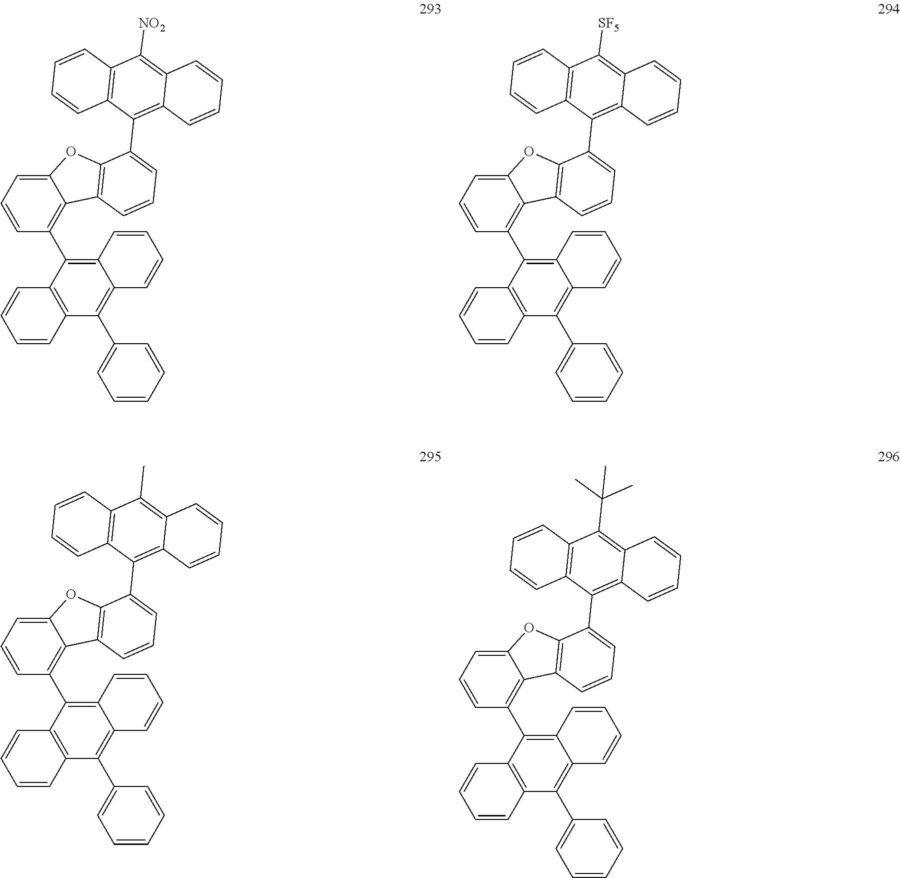

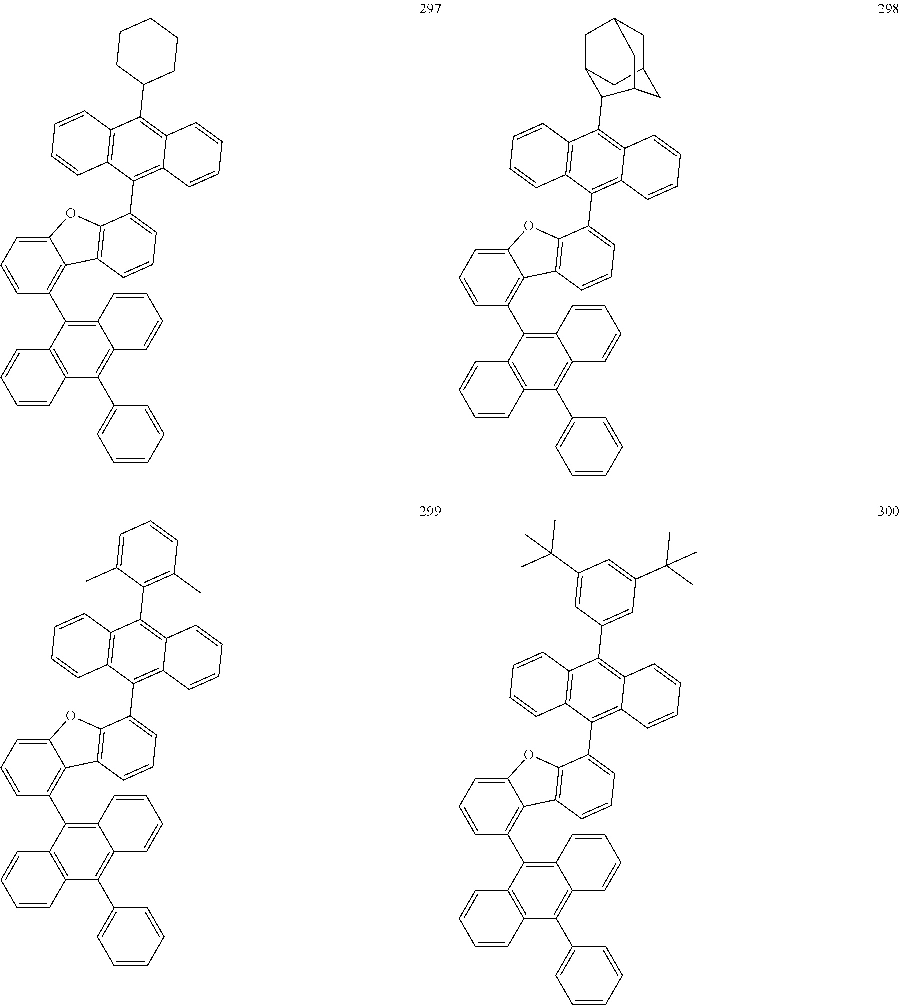 [SMILES: c1ccc(-c2c3ccccc3c(-c3cccc4oc5c(-c6c7ccccc7c(C7CCCCC7)c7ccccc67)cccc5c34)c3ccccc23)cc1 ; c1ccc(-c2c3ccccc3c(-c3cccc4oc5c(-c6c7ccccc7c(C7C8CC9CC(C8)CC7C9)c7ccccc67)cccc5c34)c3ccccc23)cc1 ; Cc1cccc(C)c1-c1c2ccccc2c(-c2cccc3c2oc2cccc(-c4c5ccccc5c(-c5ccccc5)c5ccccc45)c23)c2ccccc12 ; CC(C)(C)c1cc(-c2c3ccccc3c(-c3cccc4c3oc3cccc(-c5c6ccccc6c(-c6ccccc6)c6ccccc56)c34)c3ccccc23)cc(C(C)(C)C)c1]

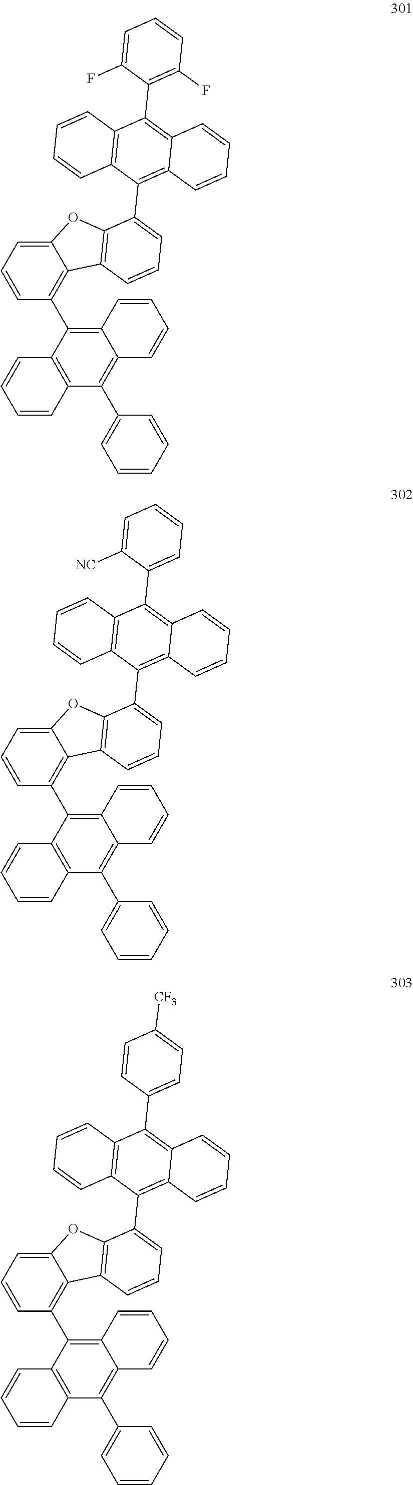

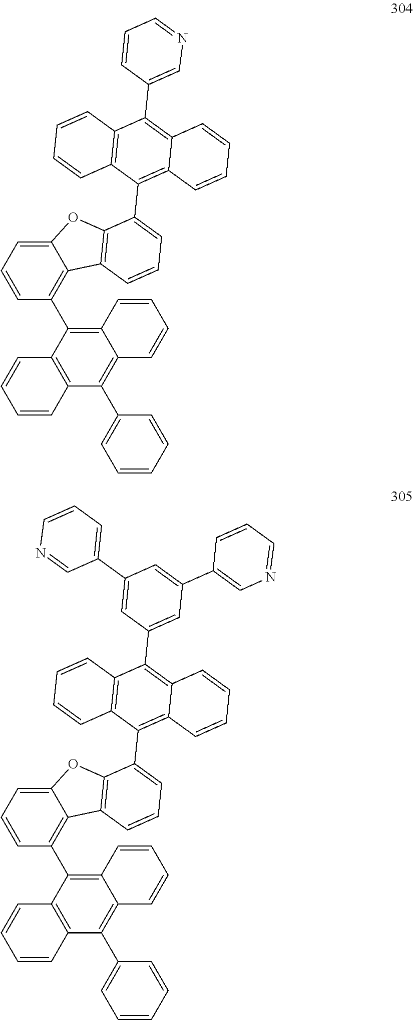 [SMILES: c1ccc(-c2c3ccccc3c(-c3cccc4oc5c(-c6c7ccccc7c(-c7cccnc7)c7ccccc67)cccc5c34)c3ccccc23)cc1 ; c1ccc(-c2c3ccccc3c(-c3cccc4oc5c(-c6c7ccccc7c(-c7cc(-c8cccnc8)cc(-c8cccnc8)c7)c7ccccc67)cccc5c34)c3ccccc23)cc1]

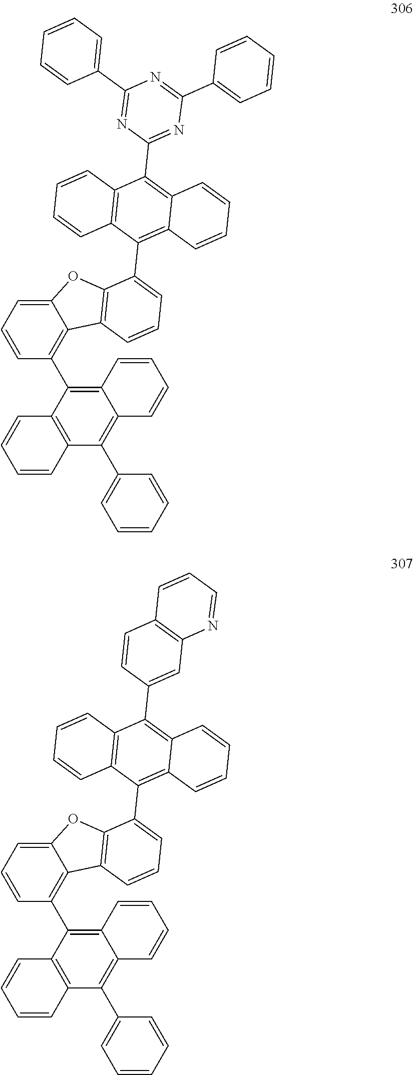 [SMILES: c1ccc(-c2nc(-c3ccccc3)nc(-c3c4ccccc4c(-c4cccc5c4oc4cccc(-c6c7ccccc7c(-c7ccccc7)c7ccccc67)c45)c4ccccc34)n2)cc1 ; c1ccc(-c2c3ccccc3c(-c3cccc4oc5c(-c6c7ccccc7c(-c7ccc8cccnc8c7)c7ccccc67)cccc5c34)c3ccccc23)cc1]

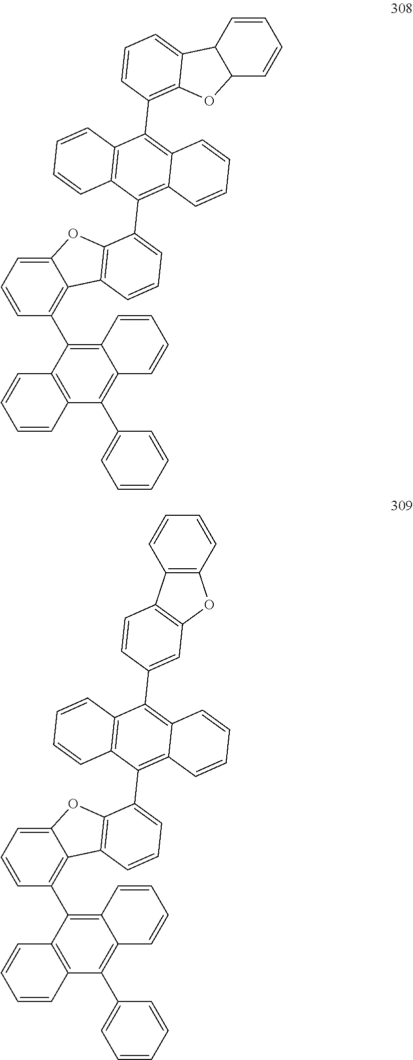

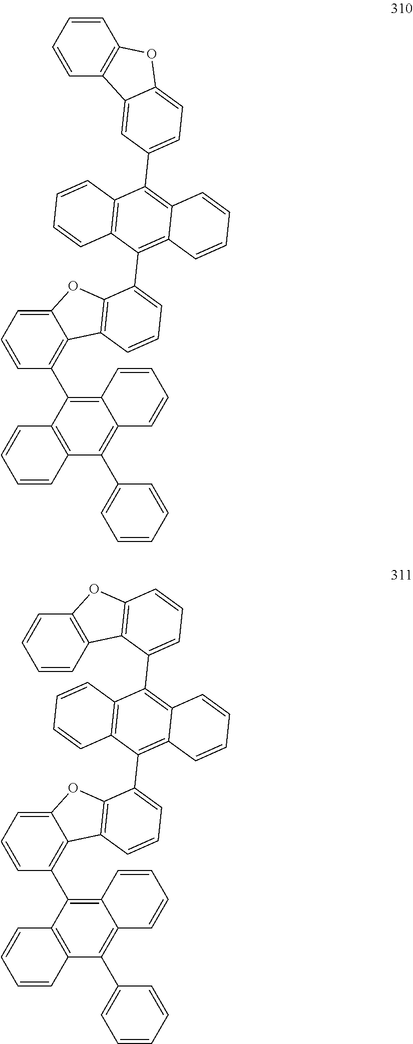 [SMILES: c1ccc(-c2c3ccccc3c(-c3cccc4oc5c(-c6c7ccccc7c(-c7ccc8oc9ccccc9c8c7)c7ccccc67)cccc5c34)c3ccccc23)cc1 ; c1ccc(-c2c3ccccc3c(-c3cccc4oc5c(-c6c7ccccc7c(-c7cccc8oc9ccccc9c78)c7ccccc67)cccc5c34)c3ccccc23)cc1]

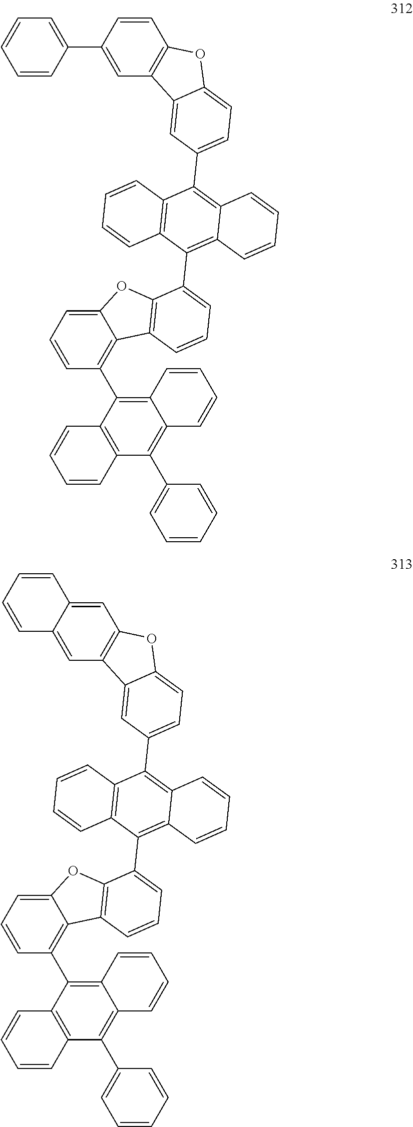 [SMILES: c1ccc(-c2ccc3oc4ccc(-c5c6ccccc6c(-c6cccc7c6oc6cccc(-c8c9ccccc9c(-c9ccccc9)c9ccccc89)c67)c6ccccc56)cc4c3c2)cc1 ; c1ccc(-c2c3ccccc3c(-c3cccc4oc5c(-c6c7ccccc7c(-c7ccc8oc9cc%10ccccc%10cc9c8c7)c7ccccc67)cccc5c34)c3ccccc23)cc1]

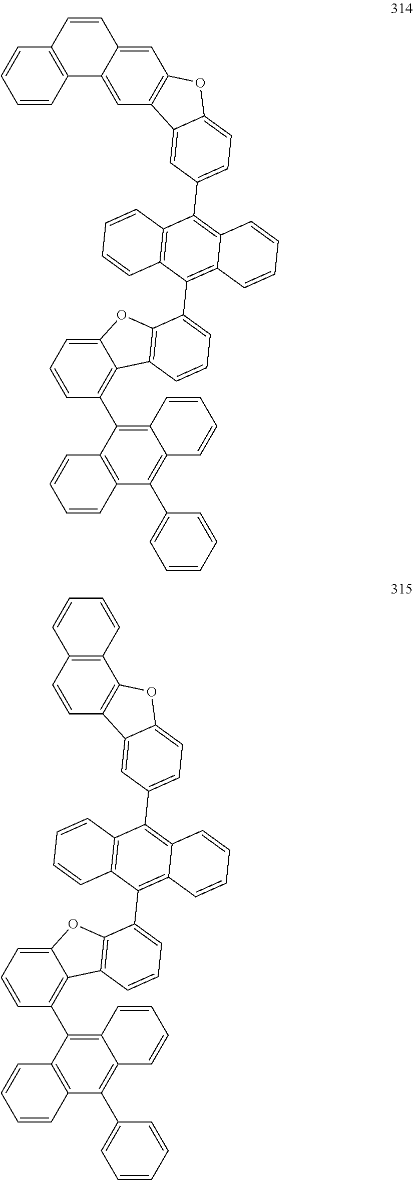 [SMILES: c1ccc(-c2c3ccccc3c(-c3cccc4oc5c(-c6c7ccccc7c(-c7ccc8oc9cc%10ccc%11ccccc%11c%10cc9c8c7)c7ccccc67)cccc5c34)c3ccccc23)cc1 ; c1ccc(-c2c3ccccc3c(-c3cccc4oc5c(-c6c7ccccc7c(-c7ccc8oc9c%10ccccc%10ccc9c8c7)c7ccccc67)cccc5c34)c3ccccc23)cc1]

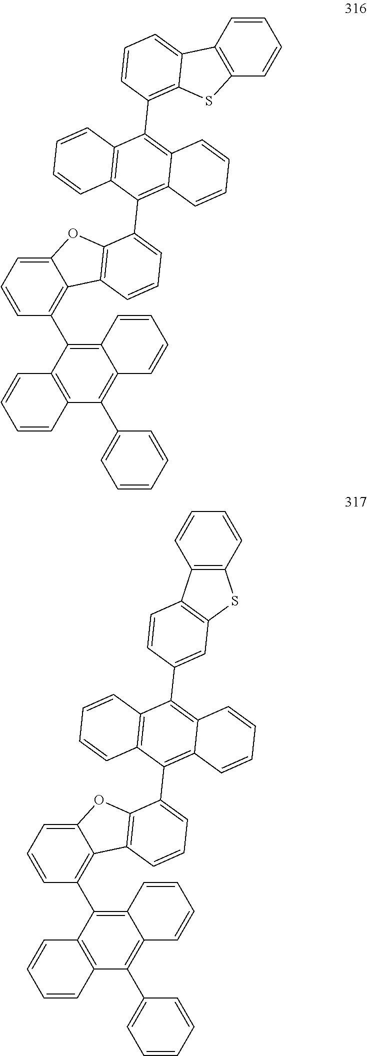

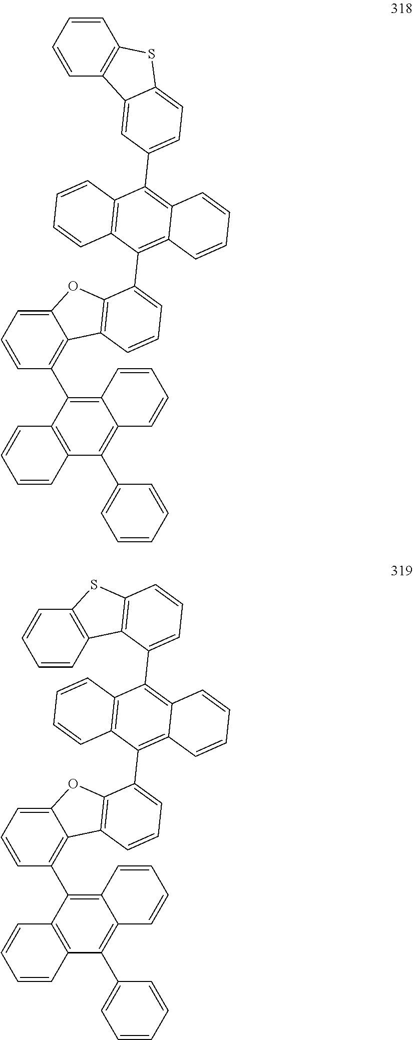

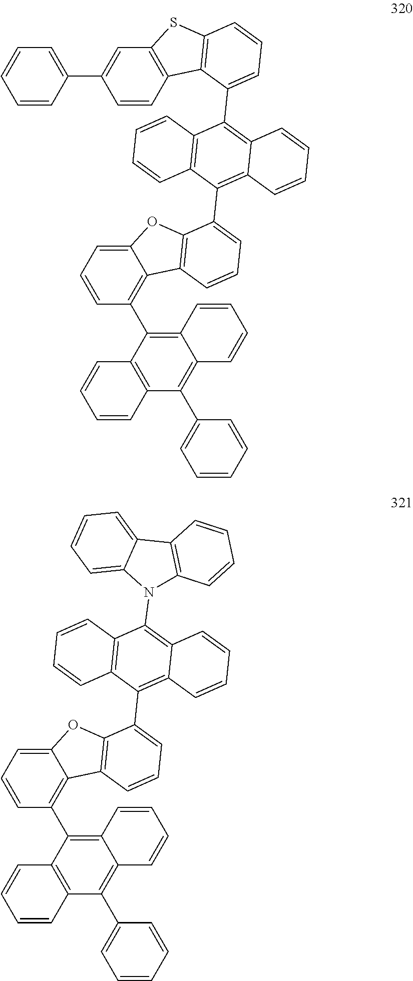

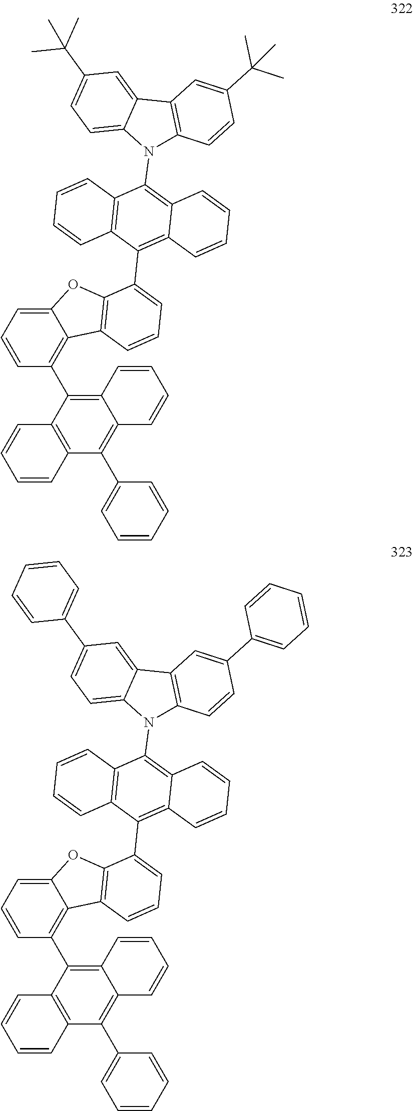 [SMILES: CC(C)(C)c1ccc2c(c1)c1cc(C(C)(C)C)ccc1n2-c1c2ccccc2c(-c2cccc3c2oc2cccc(-c4c5ccccc5c(-c5ccccc5)c5ccccc45)c23)c2ccccc12 ; c1ccc(-c2ccc3c(c2)c2cc(-c4ccccc4)ccc2n3-c2c3ccccc3c(-c3cccc4c3oc3cccc(-c5c6ccccc6c(-c6ccccc6)c6ccccc56)c34)c3ccccc23)cc1]

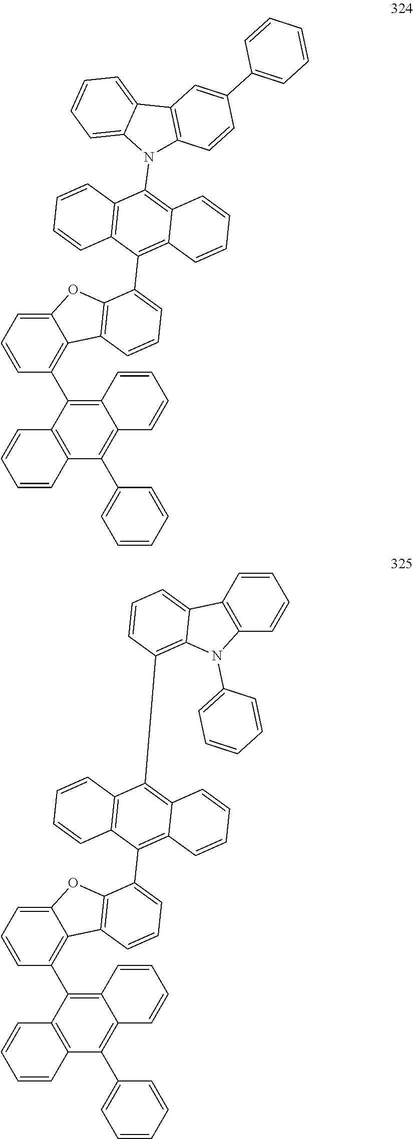

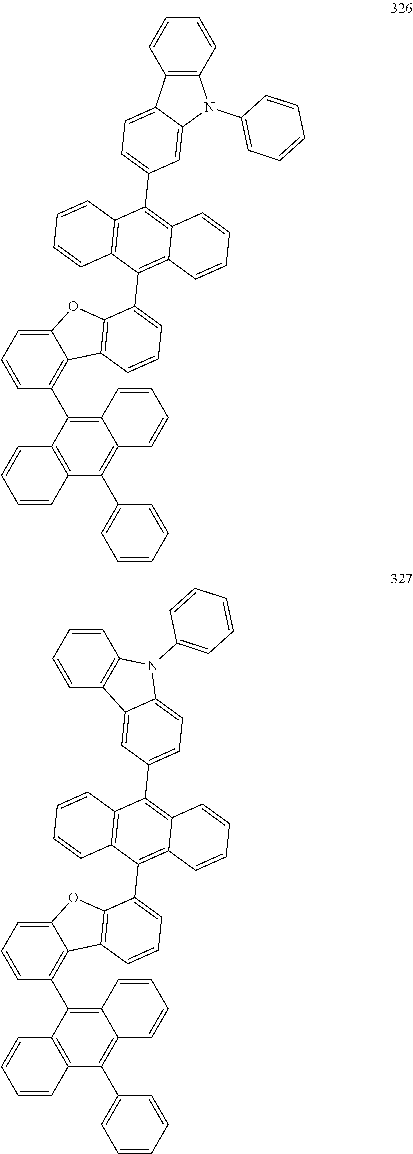 [SMILES: c1ccc(-c2c3ccccc3c(-c3cccc4oc5c(-c6c7ccccc7c(-c7ccc8c9ccccc9n(-c9ccccc9)c8c7)c7ccccc67)cccc5c34)c3ccccc23)cc1 ; c1ccc(-c2c3ccccc3c(-c3cccc4oc5c(-c6c7ccccc7c(-c7ccc8c(c7)c7ccccc7n8-c7ccccc7)c7ccccc67)cccc5c34)c3ccccc23)cc1]

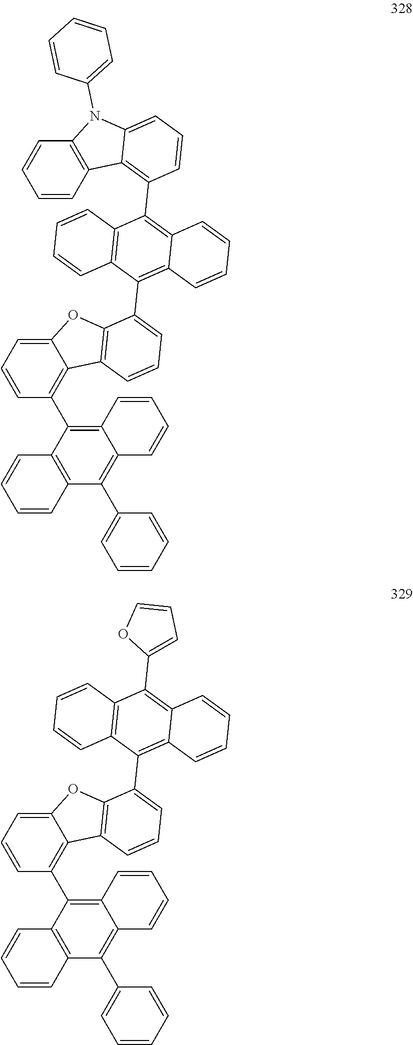

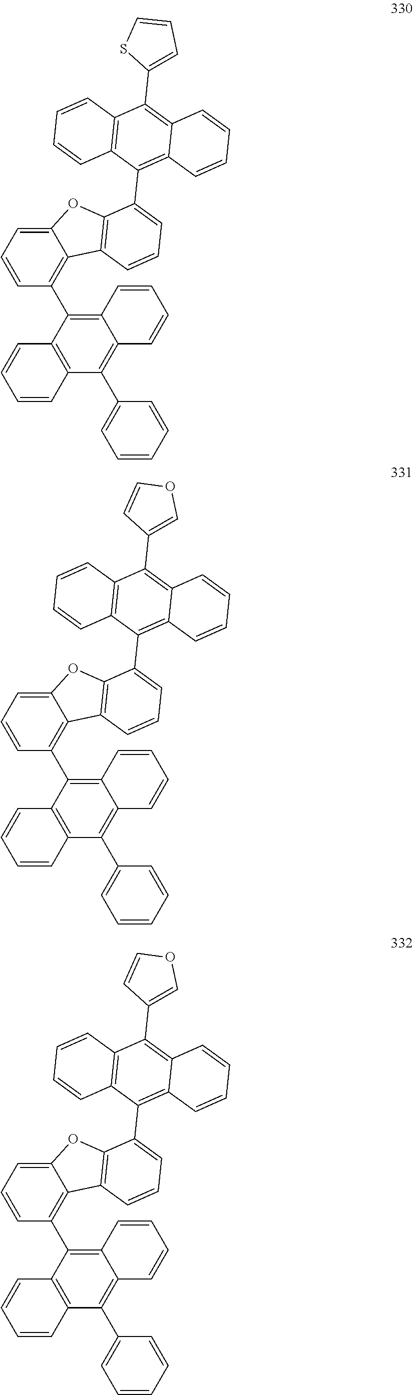

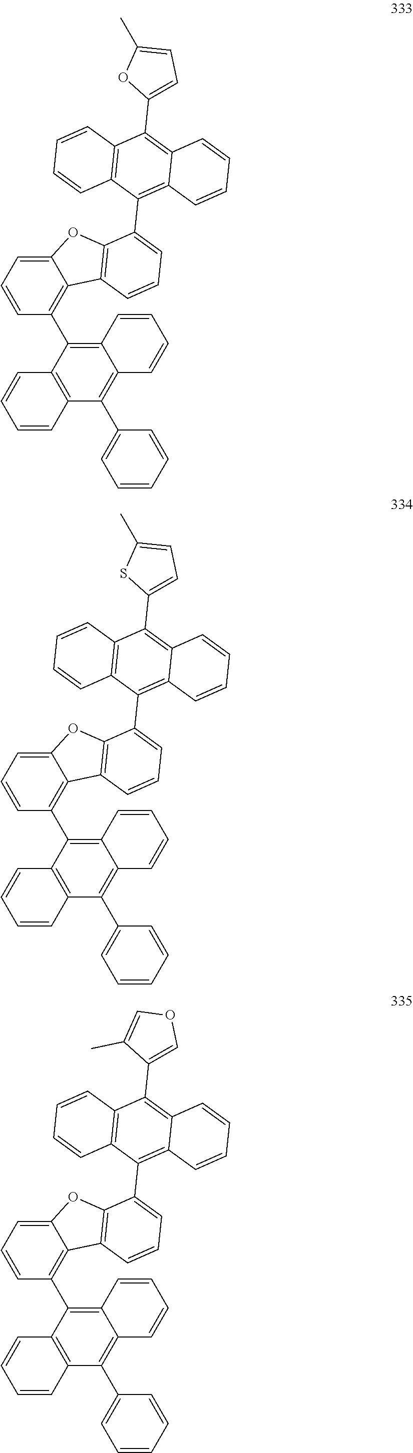

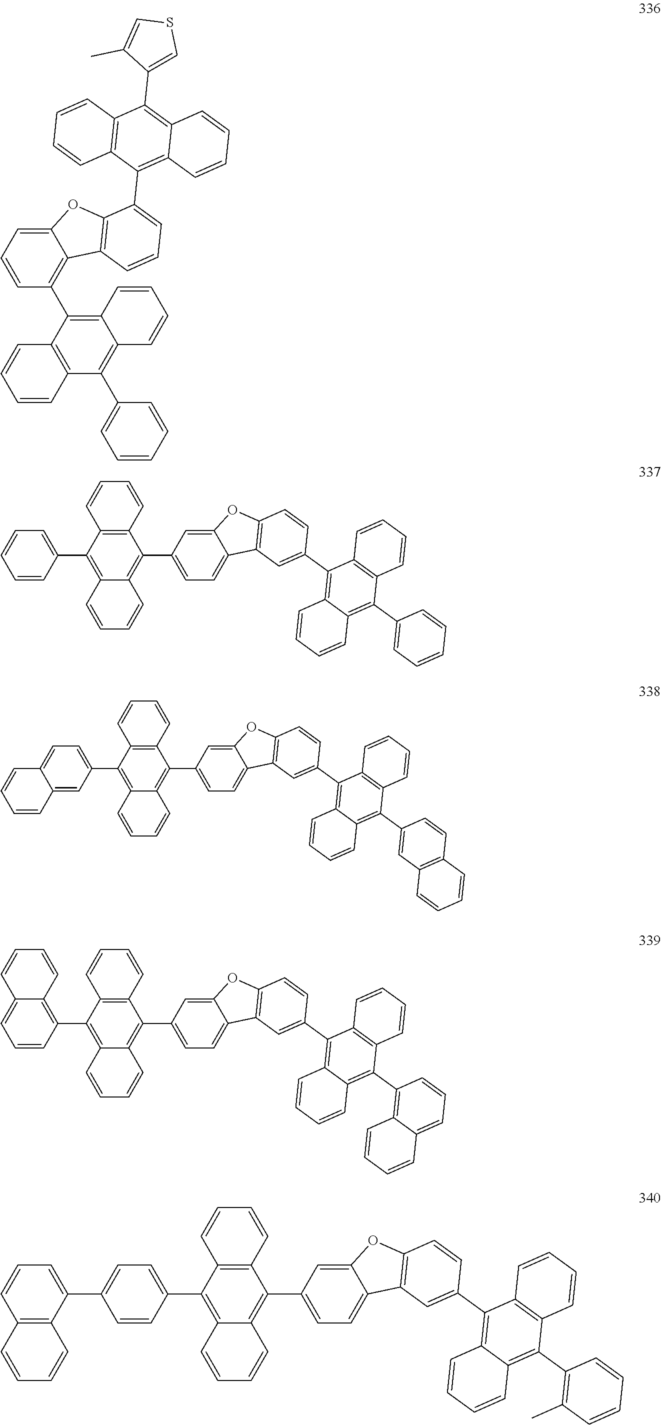

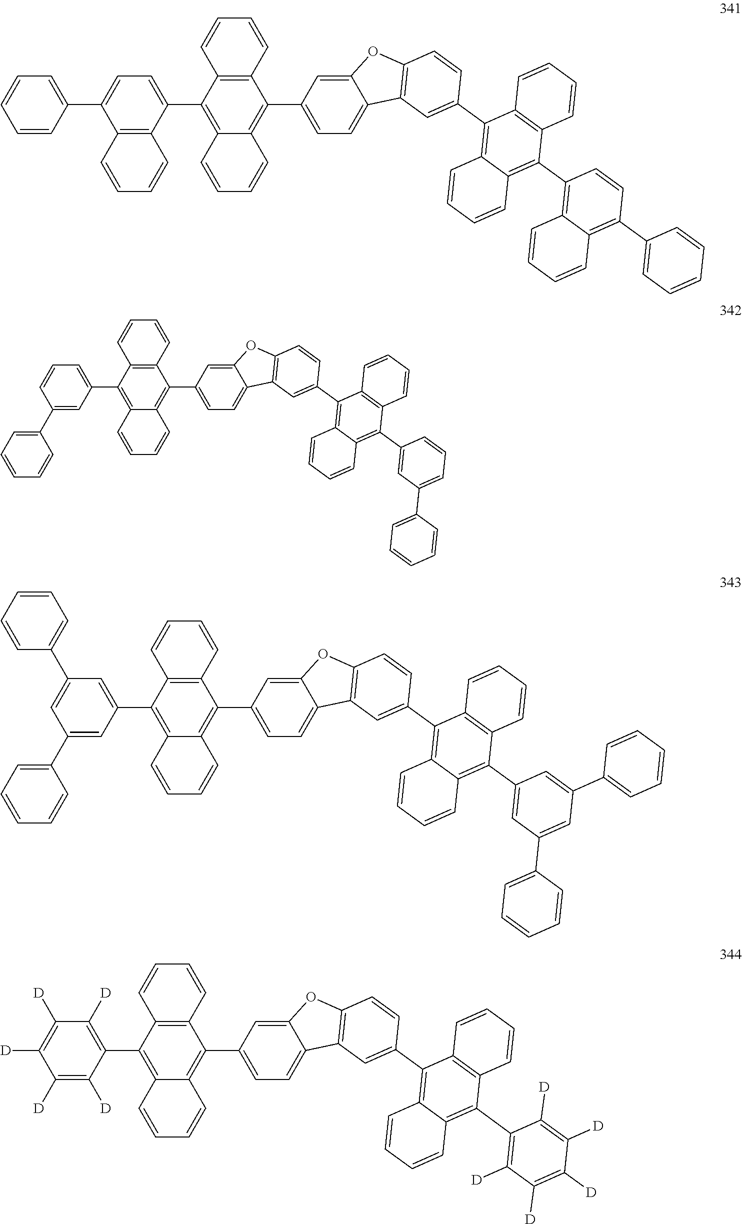

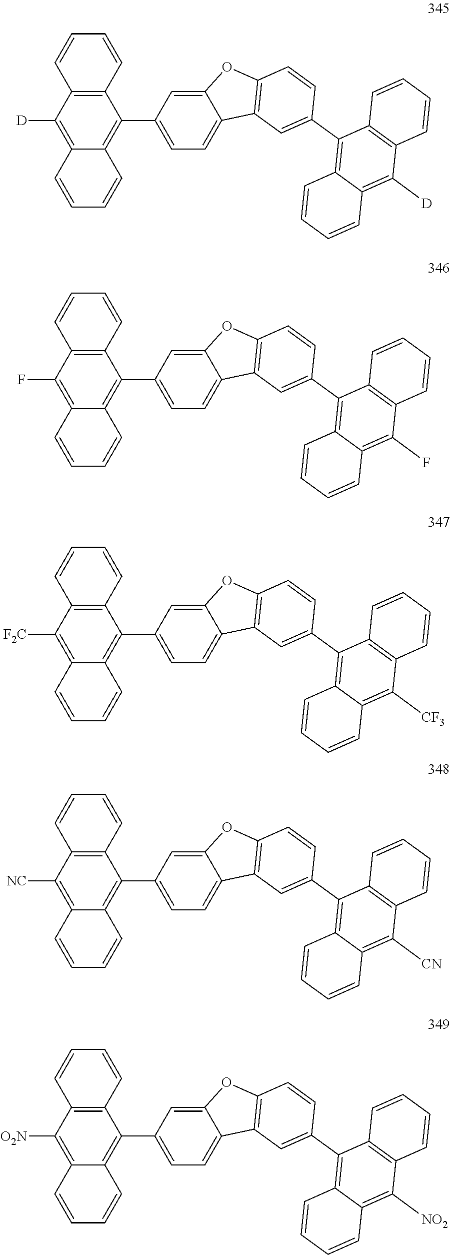

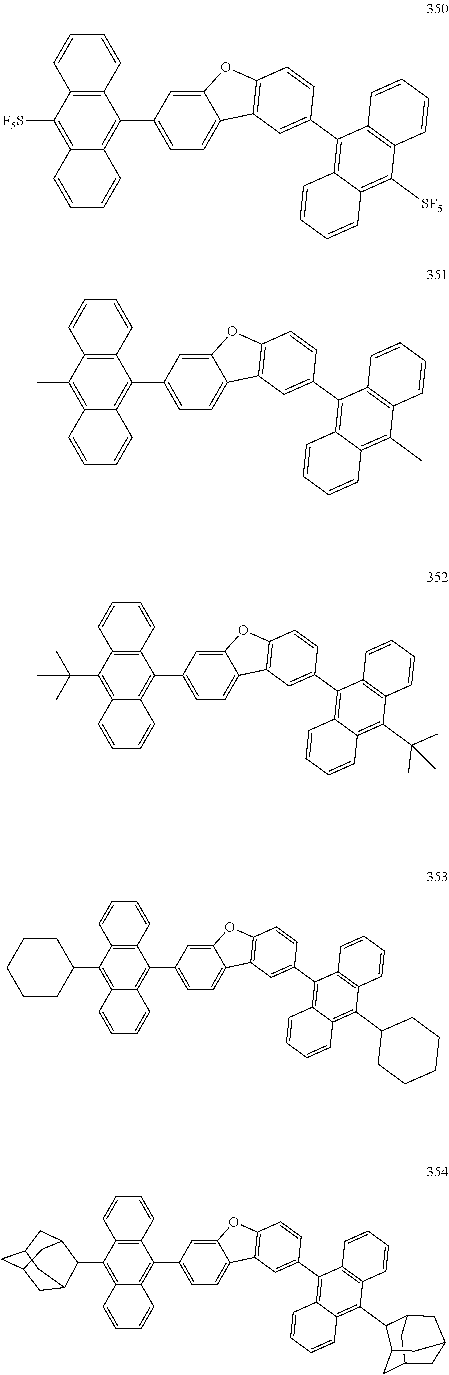

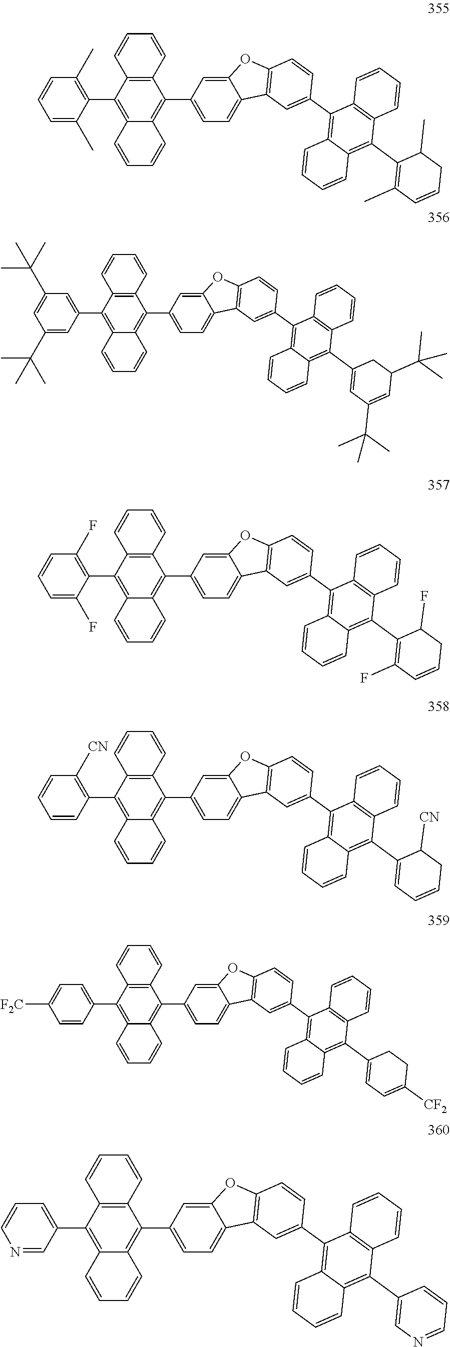

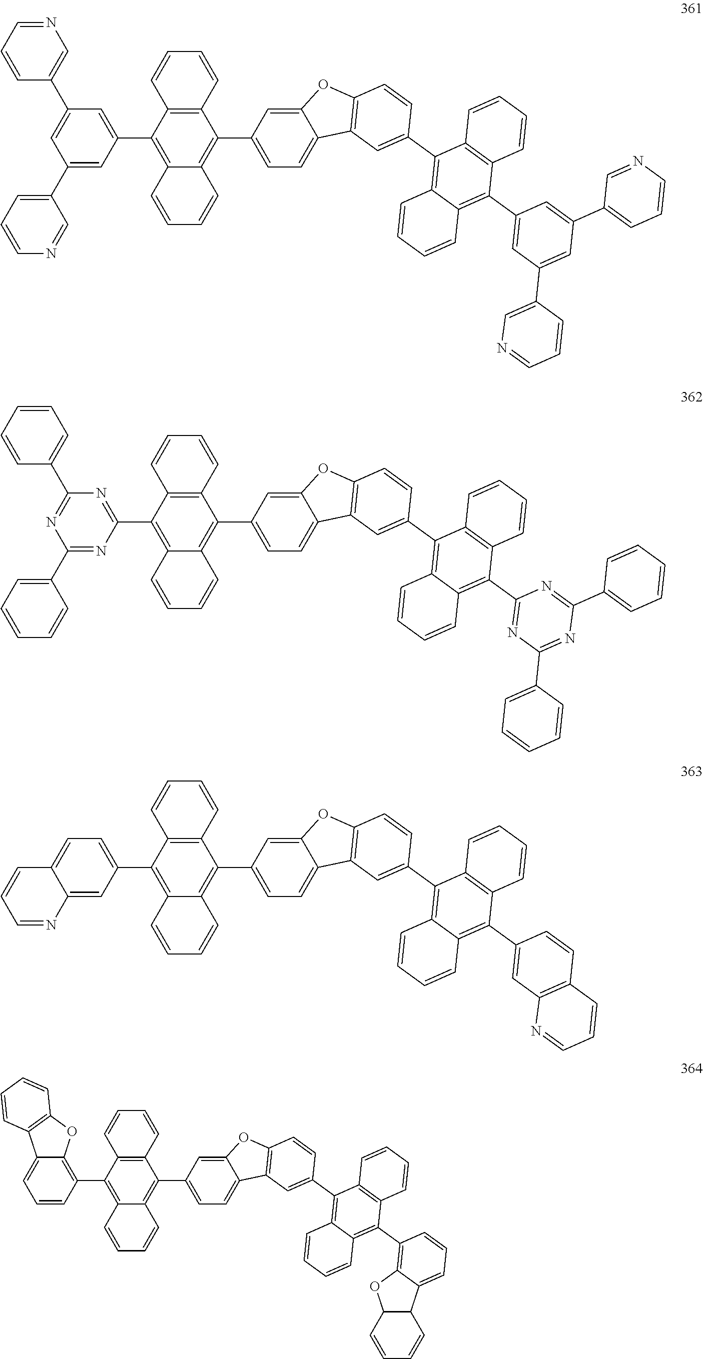 [SMILES: c1cncc(-c2cc(-c3cccnc3)cc(-c3c4ccccc4c(-c4ccc5c(c4)oc4ccc(-c6c7ccccc7c(-c7cc(-c8cccnc8)cc(-c8cccnc8)c7)c7ccccc67)cc45)c4ccccc34)c2)c1 ; c1ccc(-c2nc(-c3ccccc3)nc(-c3c4ccccc4c(-c4ccc5c(c4)oc4ccc(-c6c7ccccc7c(-c7nc(-c8ccccc8)nc(-c8ccccc8)n7)c7ccccc67)cc45)c4ccccc34)n2)cc1 ; c1cnc2cc(-c3c4ccccc4c(-c4ccc5c(c4)oc4ccc(-c6c7ccccc7c(-c7ccc8cccnc8c7)c7ccccc67)cc45)c4ccccc34)ccc2c1 ; C1=CC2Oc3c(-c4c5ccccc5c(-c5ccc6oc7cc(-c8c9ccccc9c(-c9cccc%10c9oc9ccccc9%10)c9ccccc89)ccc7c6c5)c5ccccc45)cccc3C2C=C1]

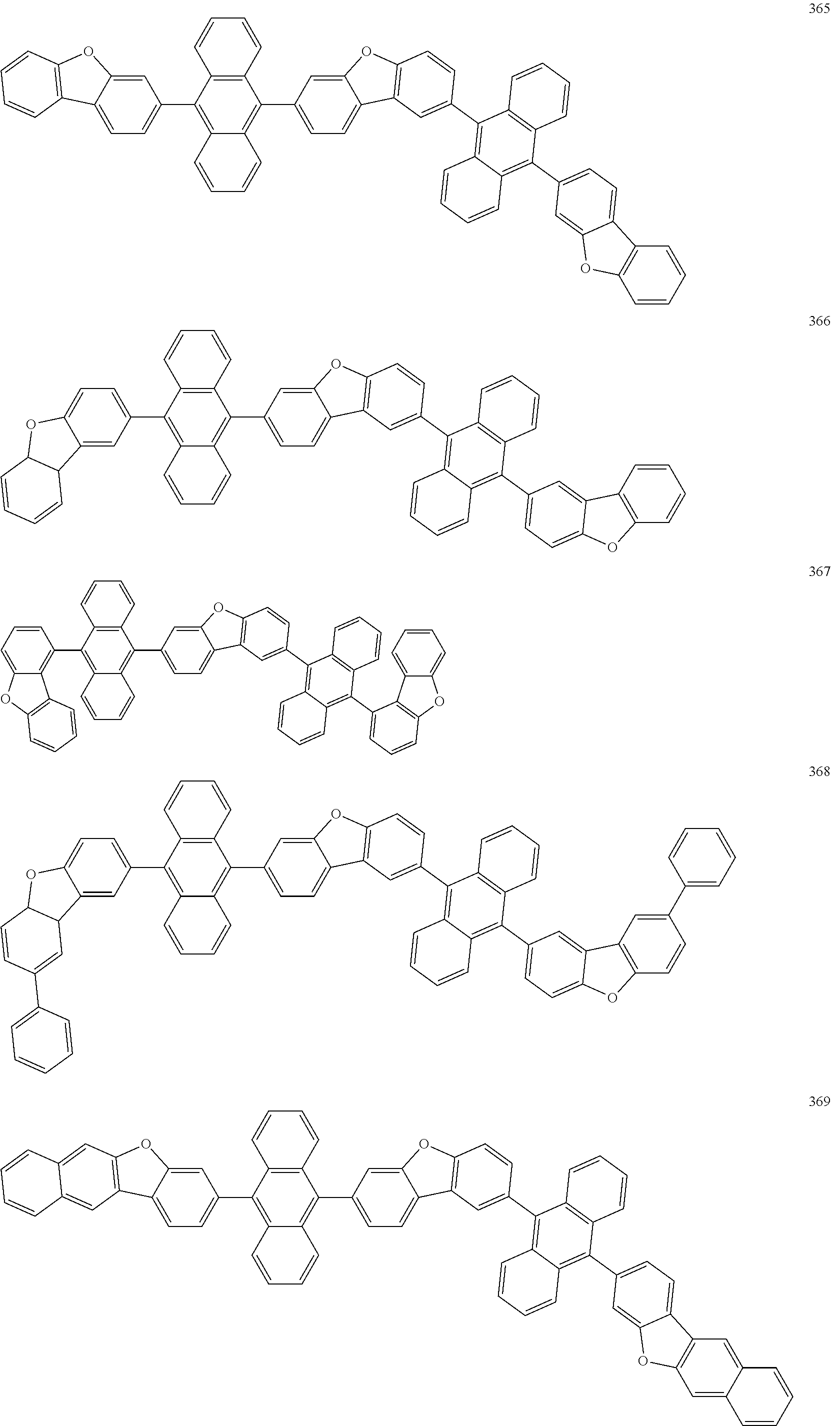

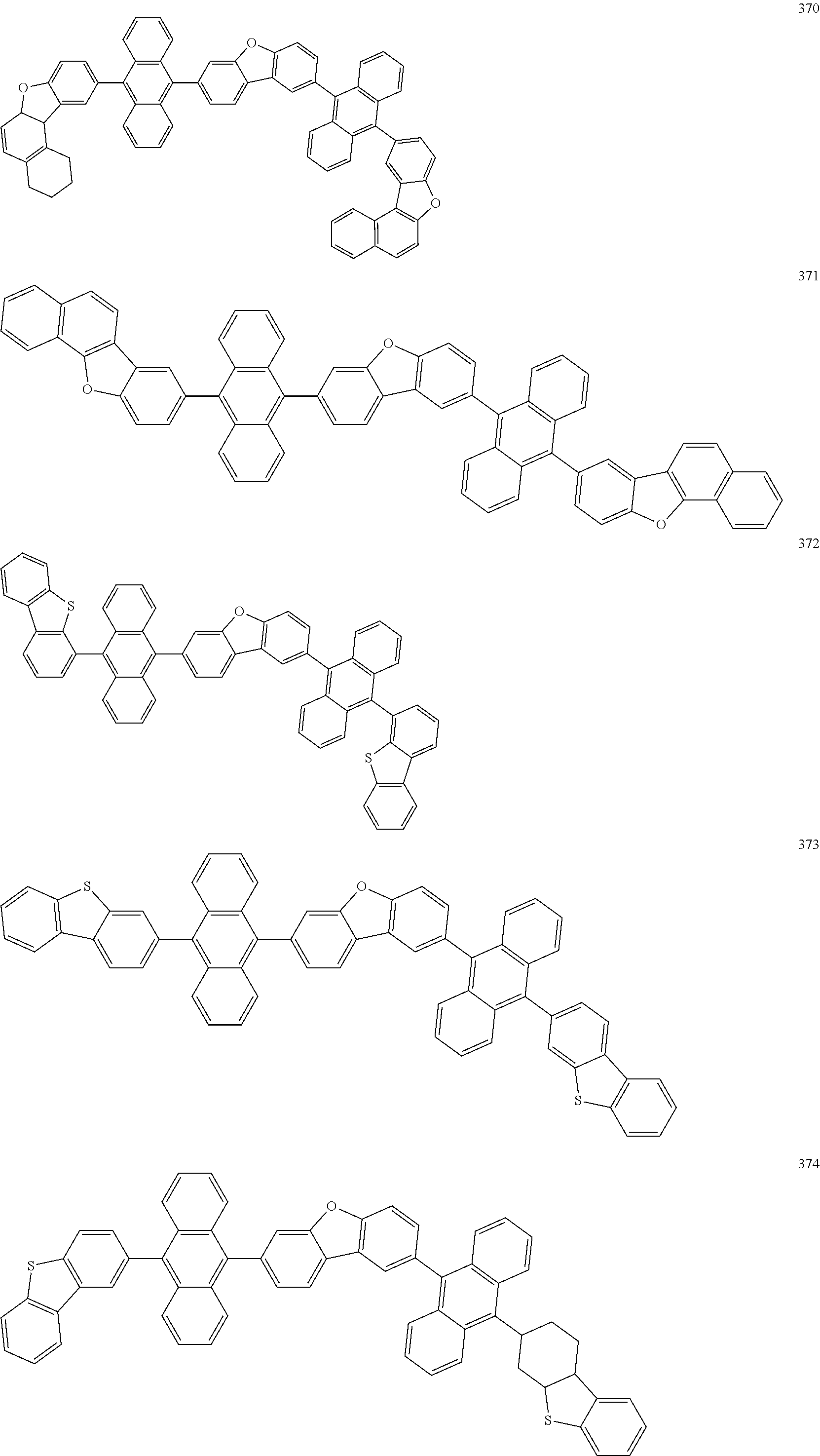

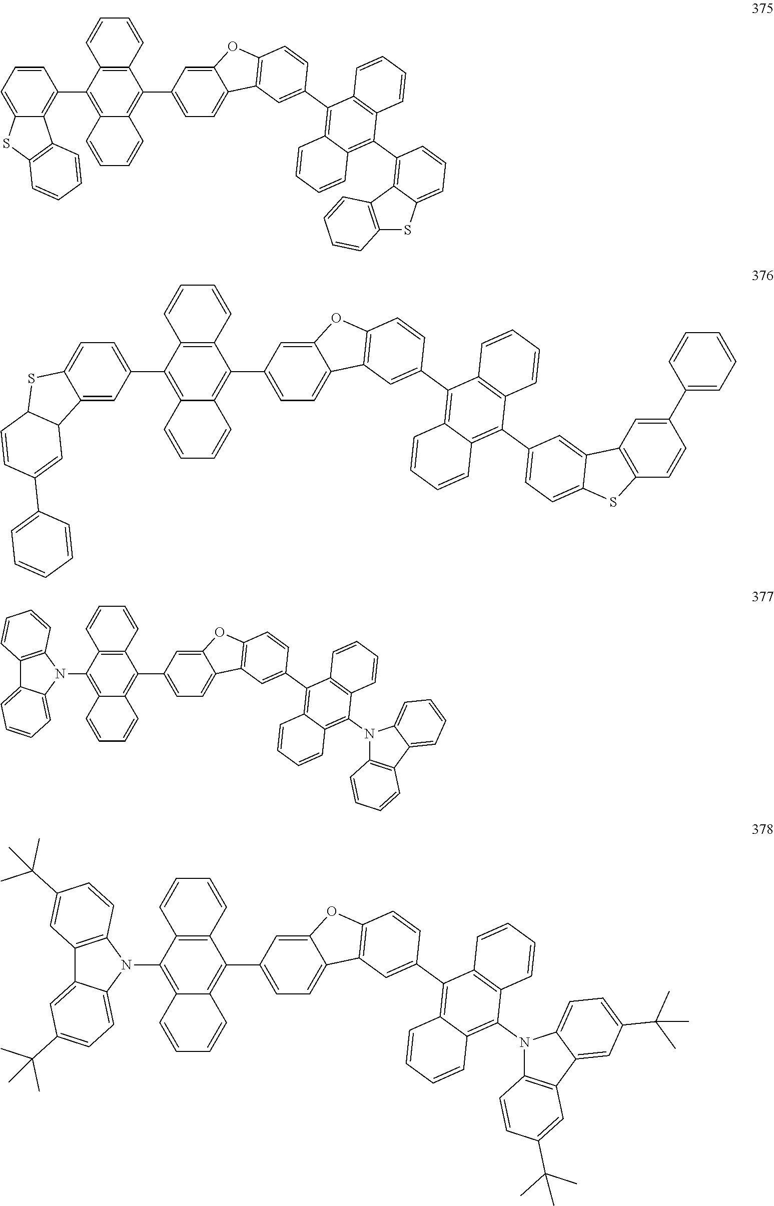

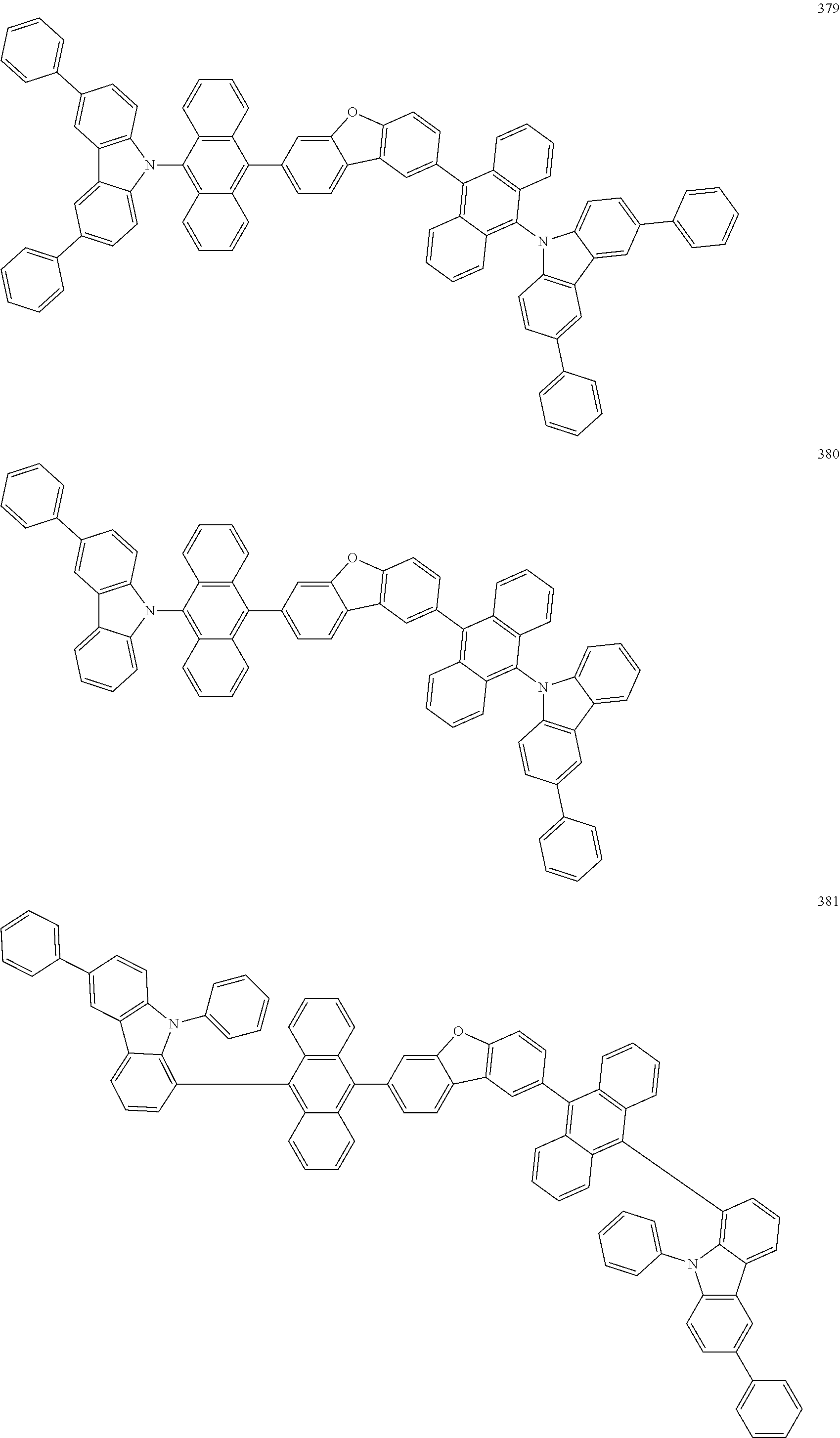 [SMILES: c1ccc(-c2ccc3c(c2)c2cc(-c4ccccc4)ccc2n3-c2c3ccccc3c(-c3ccc4c(c3)oc3ccc(-c5c6ccccc6c(-n6c7ccc(-c8ccccc8)cc7c7cc(-c8ccccc8)ccc76)c6ccccc56)cc34)c3ccccc23)cc1 ; c1ccc(-c2ccc3c(c2)c2ccccc2n3-c2c3ccccc3c(-c3ccc4c(c3)oc3ccc(-c5c6ccccc6c(-n6c7ccccc7c7cc(-c8ccccc8)ccc76)c6ccccc56)cc34)c3ccccc23)cc1 ; c1ccc(-c2ccc3c(c2)c2cccc(-c4c5ccccc5c(-c5ccc6c(c5)oc5ccc(-c7c8ccccc8c(-c8cccc9c%10cc(-c%11ccccc%11)ccc%10n(-c%10ccccc%10)c89)c8ccccc78)cc56)c5ccccc45)c2n3-c2ccccc2)cc1]

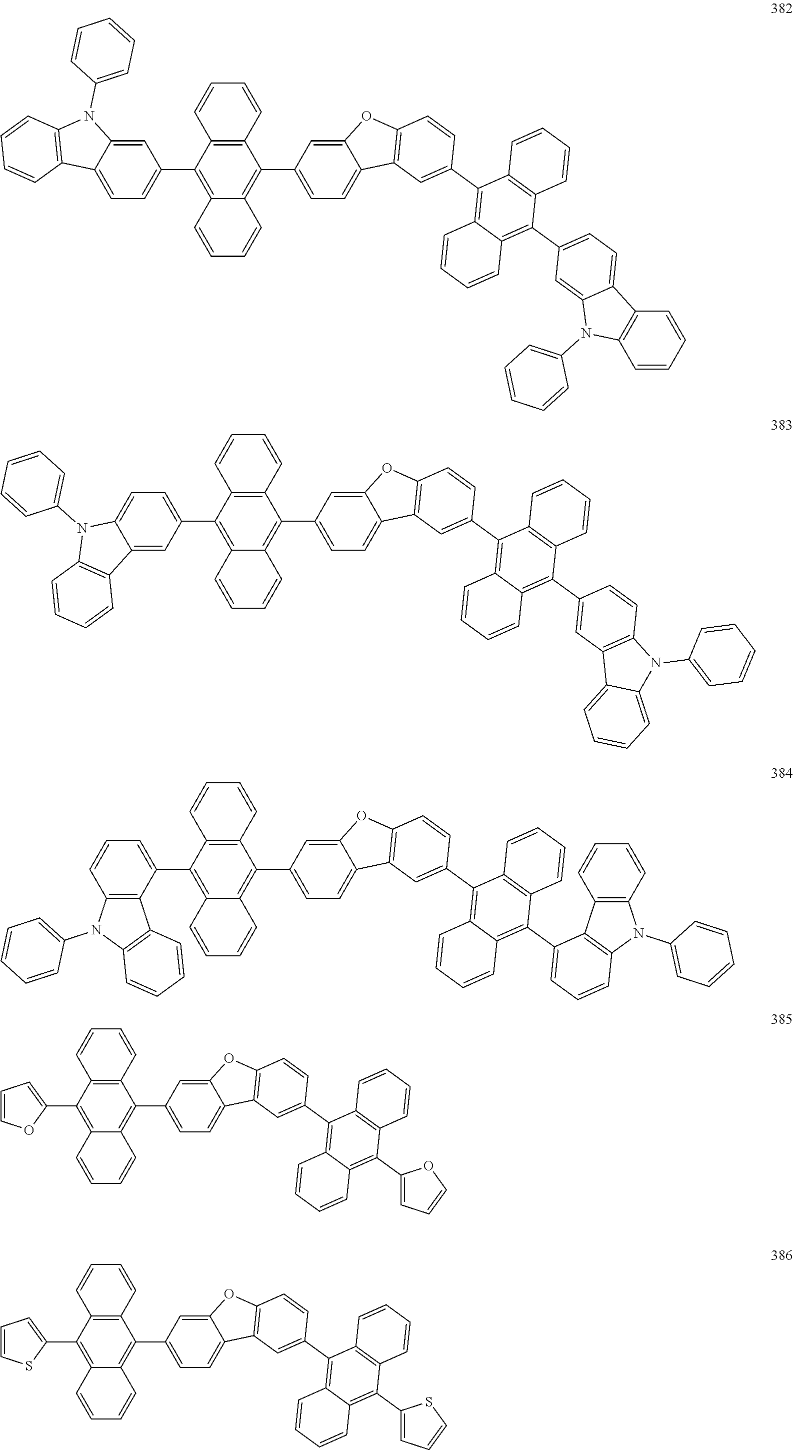

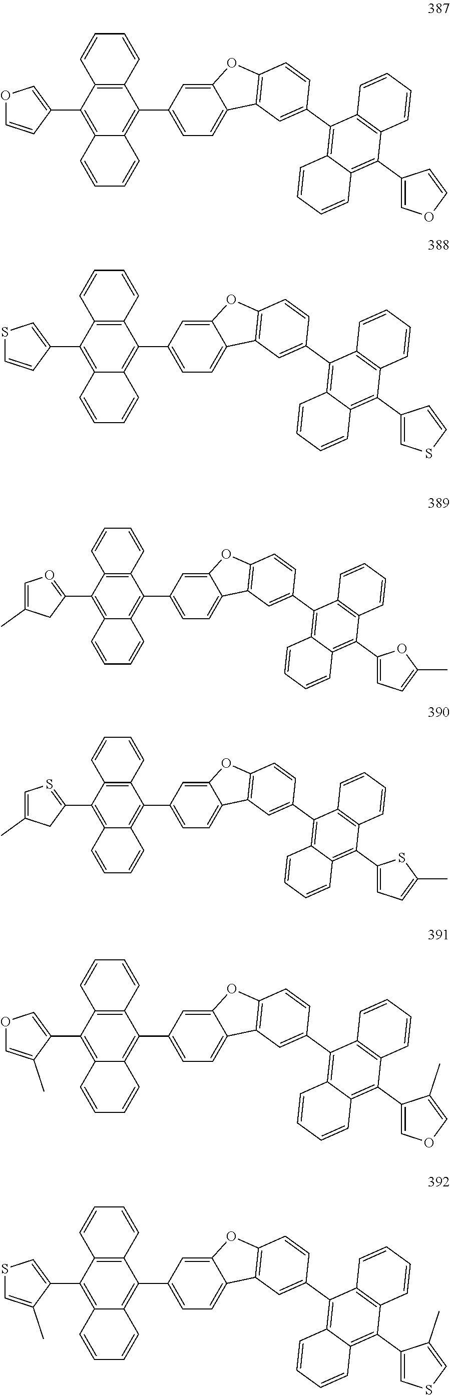

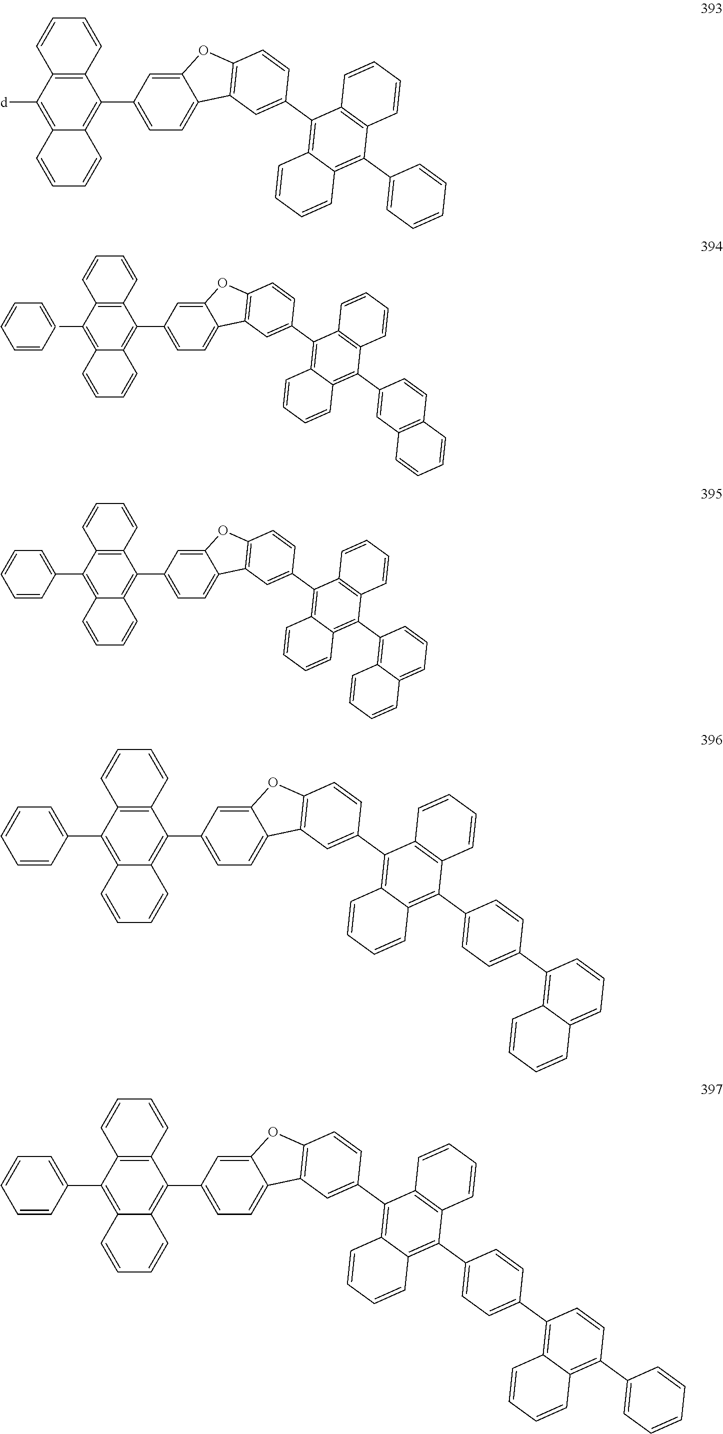

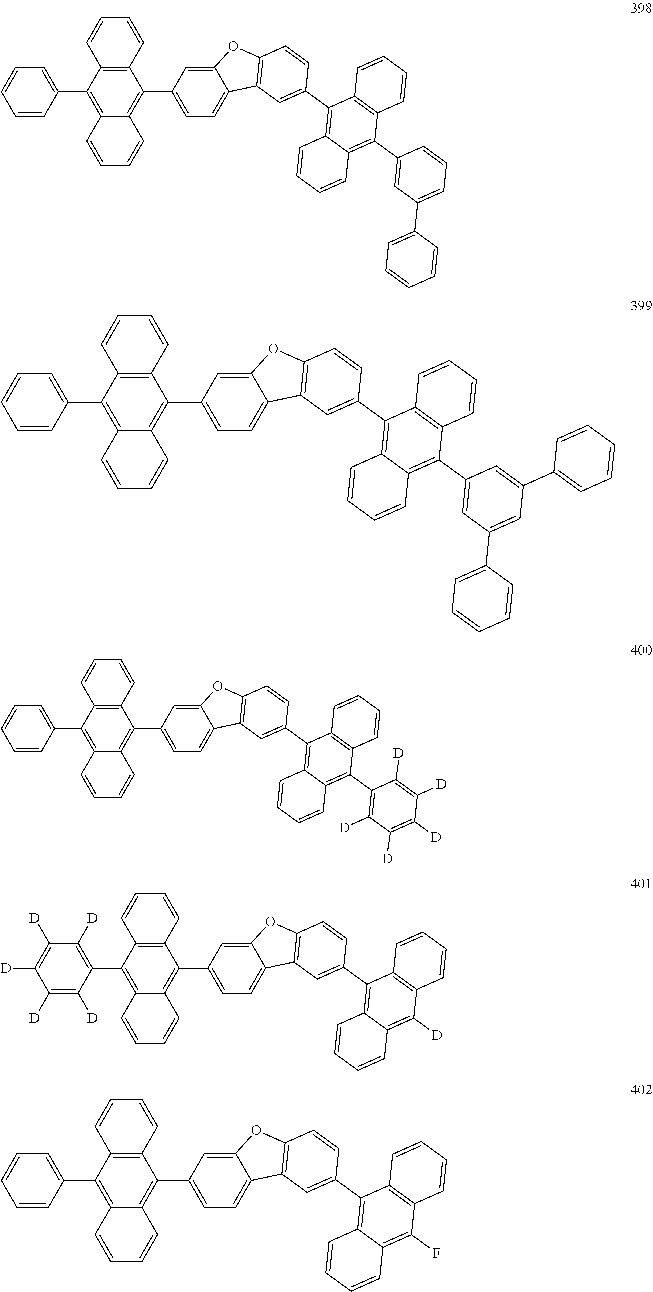

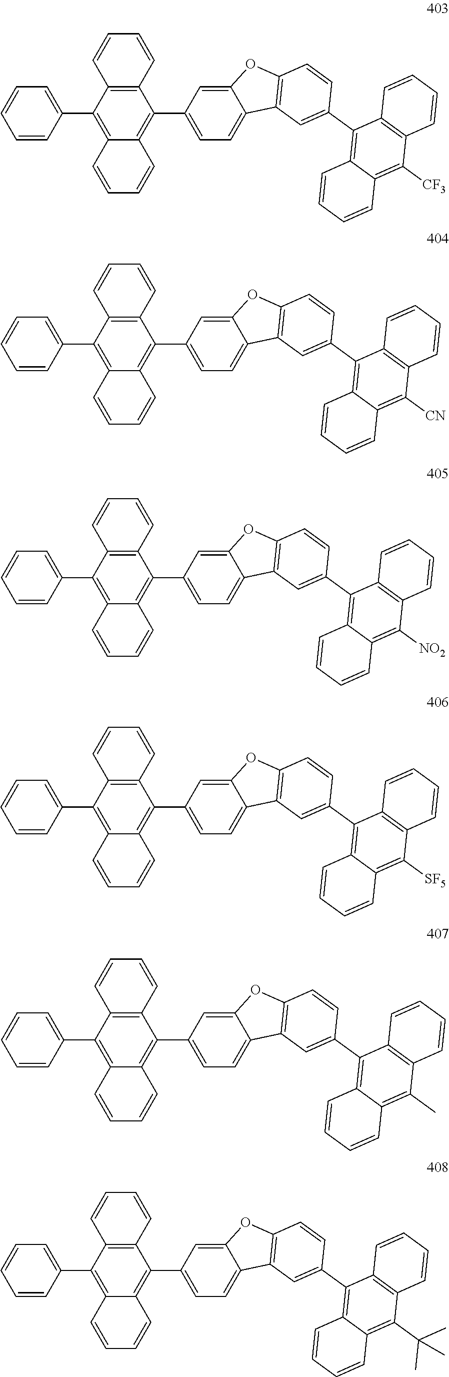

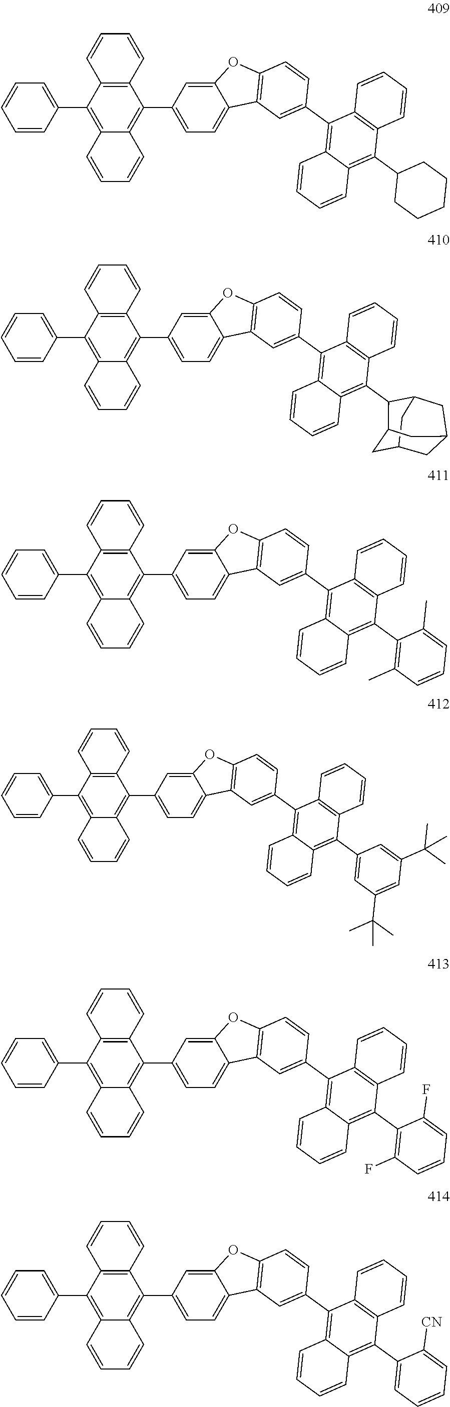

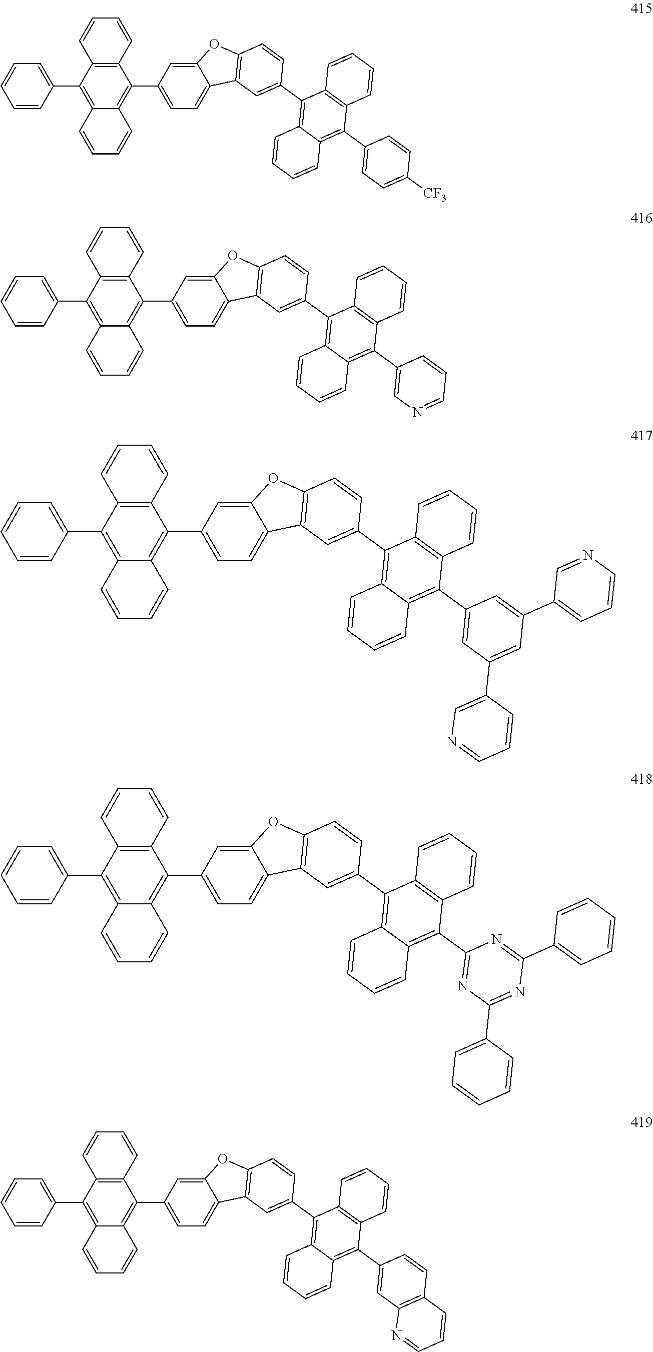

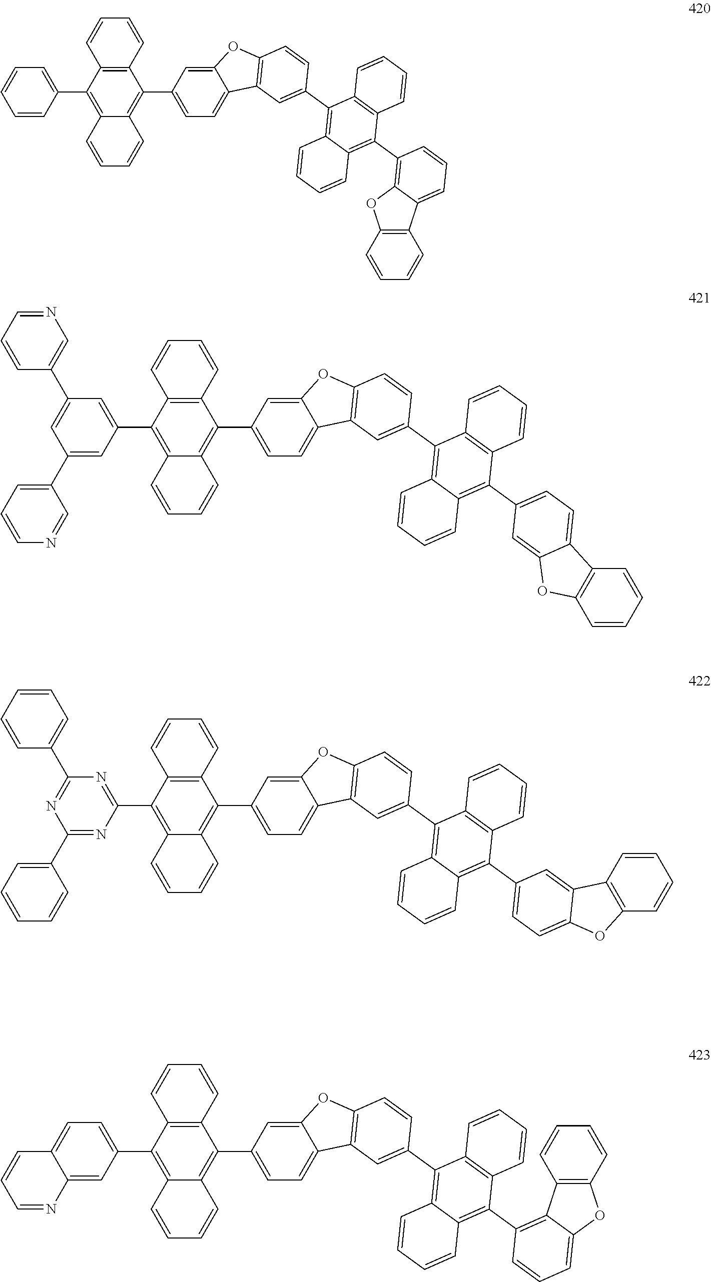 [SMILES: c1ccc(-c2c3ccccc3c(-c3ccc4c(c3)oc3ccc(-c5c6ccccc6c(-c6cccc7c6oc6ccccc67)c6ccccc56)cc34)c3ccccc23)cc1 ; c1cncc(-c2cc(-c3cccnc3)cc(-c3c4ccccc4c(-c4ccc5c(c4)oc4ccc(-c6c7ccccc7c(-c7ccc8c(c7)oc7ccccc78)c7ccccc67)cc45)c4ccccc34)c2)c1 ; c1ccc(-c2nc(-c3ccccc3)nc(-c3c4ccccc4c(-c4ccc5c(c4)oc4ccc(-c6c7ccccc7c(-c7ccc8oc9ccccc9c8c7)c7ccccc67)cc45)c4ccccc34)n2)cc1 ; c1cnc2cc(-c3c4ccccc4c(-c4ccc5c(c4)oc4ccc(-c6c7ccccc7c(-c7cccc8oc9ccccc9c78)c7ccccc67)cc45)c4ccccc34)ccc2c1]

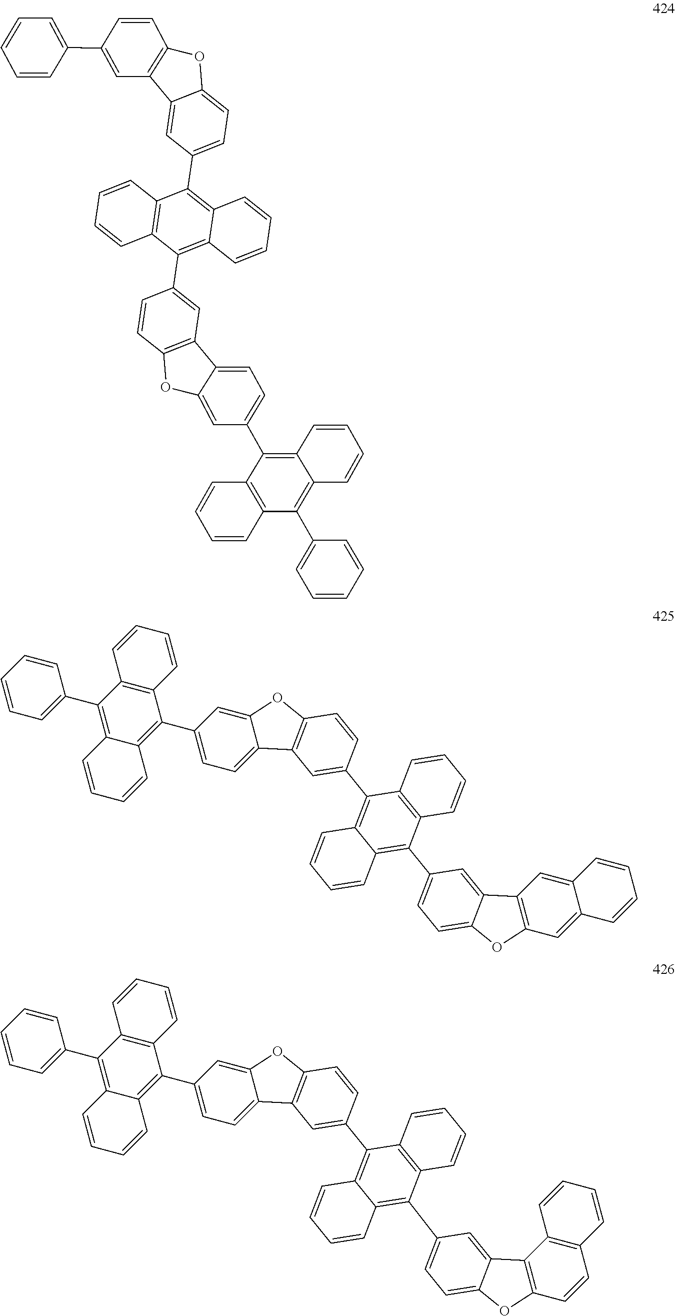 [SMILES: c1ccc(-c2ccc3oc4ccc(-c5c6ccccc6c(-c6ccc7oc8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)oc3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3ccc4c(c3)oc3ccc(-c5c6ccccc6c(-c6ccc7oc8ccc9ccccc9c8c7c6)c6ccccc56)cc34)c3ccccc23)cc1]

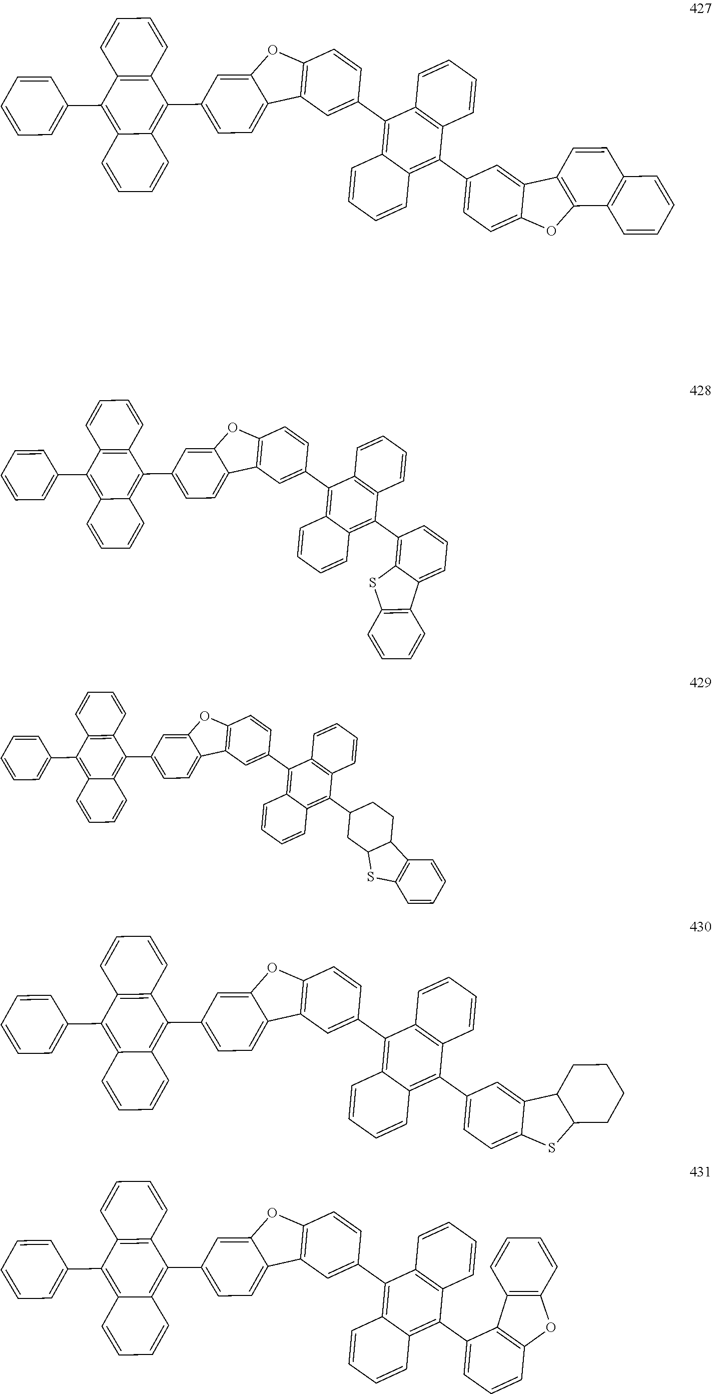

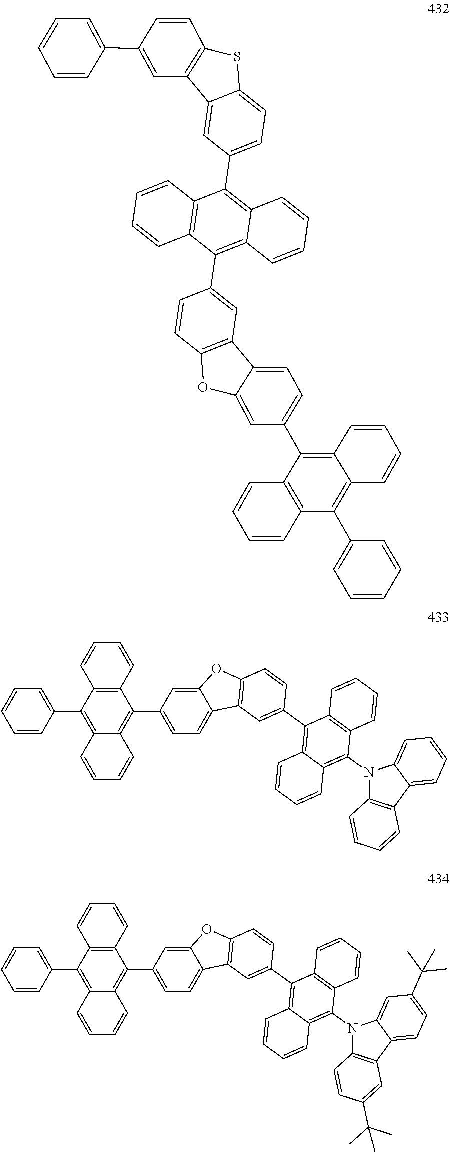 [SMILES: c1ccc(-c2ccc3sc4ccc(-c5c6ccccc6c(-c6ccc7oc8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)oc3ccc(-c5c6ccccc6c(-n6c7ccccc7c7ccccc76)c6ccccc56)cc34)c3ccccc23)cc1 ; CC(C)(C)c1ccc2c(c1)c1ccc(C(C)(C)C)cc1n2-c1c2ccccc2c(-c2ccc3oc4cc(-c5c6ccccc6c(-c6ccccc6)c6ccccc56)ccc4c3c2)c2ccccc12]

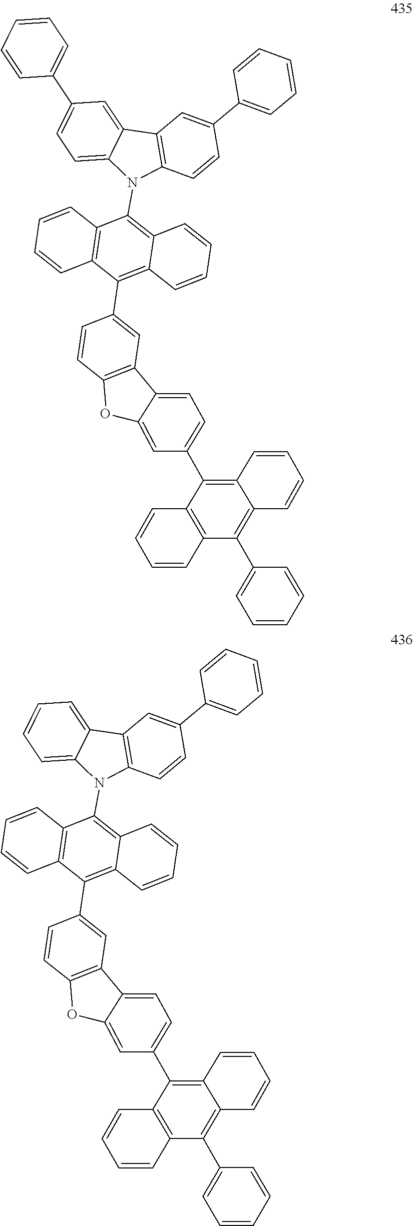

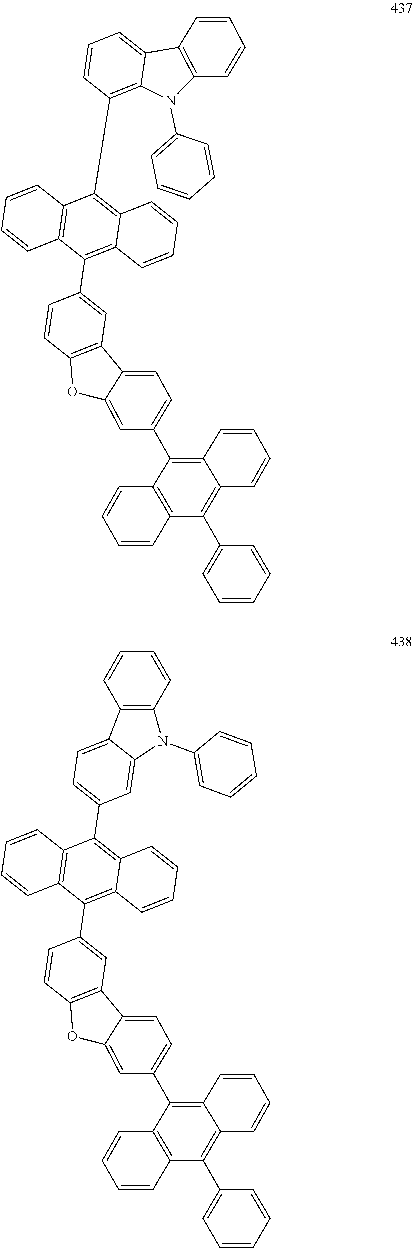 [SMILES: c1ccc(-c2c3ccccc3c(-c3ccc4c(c3)oc3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)oc3ccc(-c5c6ccccc6c(-c6ccc7c8ccccc8n(-c8ccccc8)c7c6)c6ccccc56)cc34)c3ccccc23)cc1]

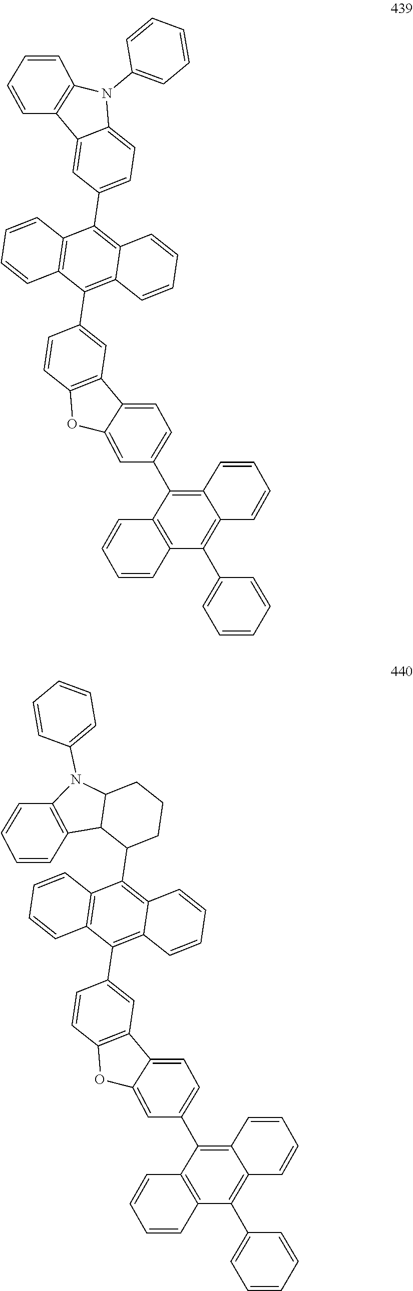 [SMILES: c1ccc(-c2c3ccccc3c(-c3ccc4c(c3)oc3ccc(-c5c6ccccc6c(-c6ccc7c(c6)c6ccccc6n7-c6ccccc6)c6ccccc56)cc34)c3ccccc23)cc1 ; c1ccc(-c2c3ccccc3c(-c3ccc4c(c3)oc3ccc(-c5c6ccccc6c(C6CCCC7C6c6ccccc6N7c6ccccc6)c6ccccc56)cc34)c3ccccc23)cc1]

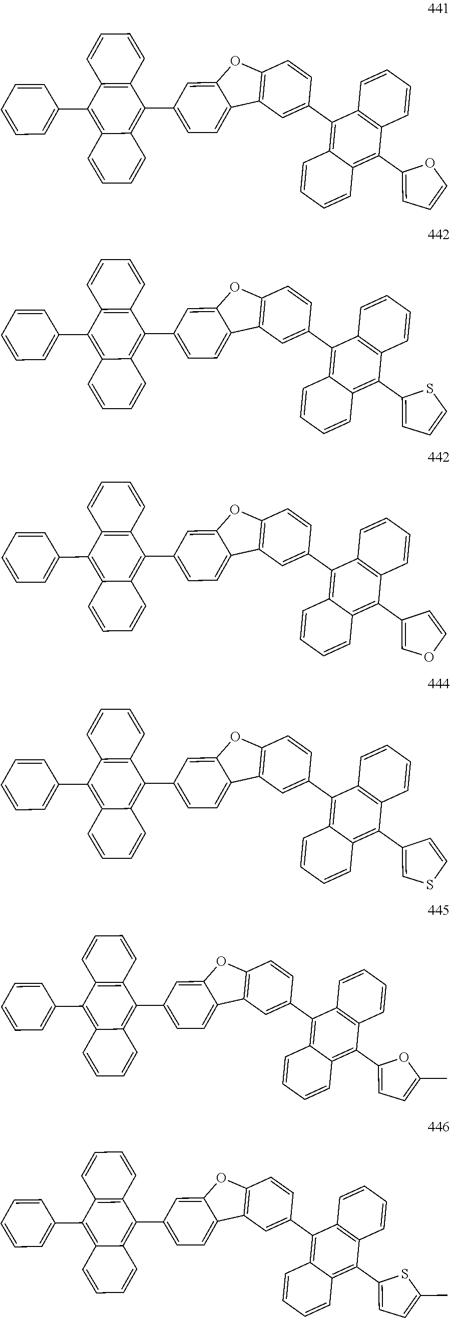

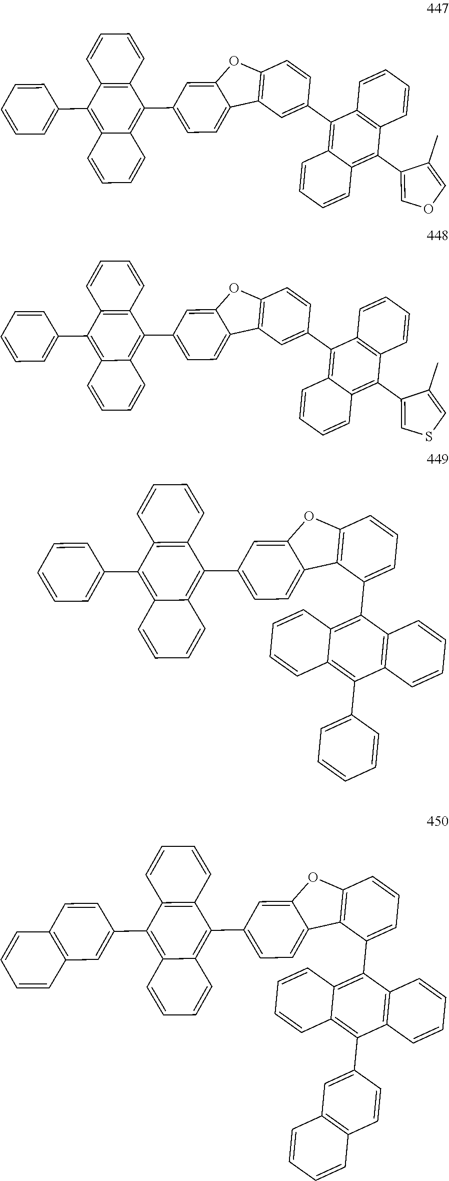 [SMILES: Cc1cocc1-c1c2ccccc2c(-c2ccc3oc4cc(-c5c6ccccc6c(-c6ccccc6)c6ccccc56)ccc4c3c2)c2ccccc12 ; Cc1cscc1-c1c2ccccc2c(-c2ccc3oc4cc(-c5c6ccccc6c(-c6ccccc6)c6ccccc56)ccc4c3c2)c2ccccc12 ; c1ccc(-c2c3ccccc3c(-c3ccc4c(c3)oc3cccc(-c5c6ccccc6c(-c6ccccc6)c6ccccc56)c34)c3ccccc23)cc1 ; c1ccc2cc(-c3c4ccccc4c(-c4ccc5c(c4)oc4cccc(-c6c7ccccc7c(-c7ccc8ccccc8c7)c7ccccc67)c45)c4ccccc34)ccc2c1]

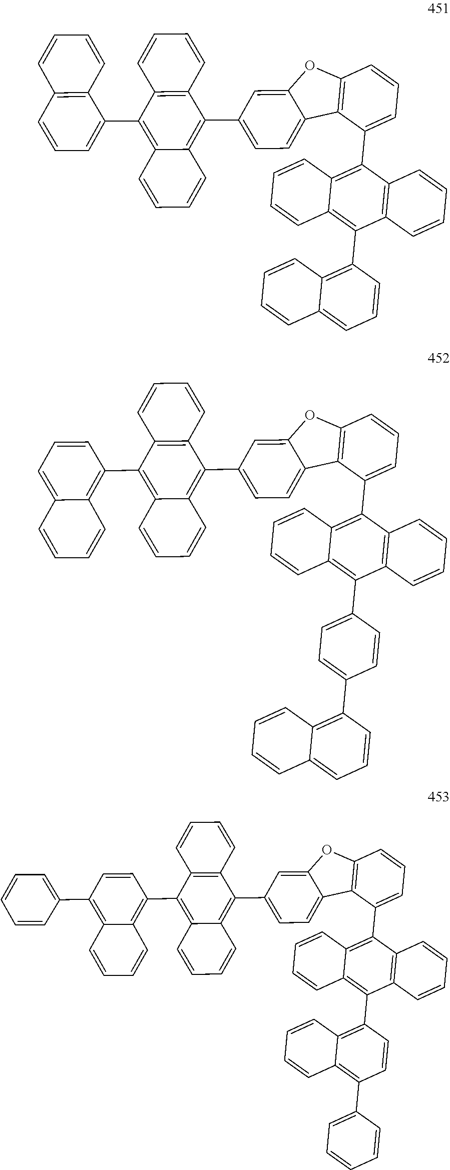 [SMILES: c1ccc2c(-c3c4ccccc4c(-c4ccc5c(c4)oc4cccc(-c6c7ccccc7c(-c7cccc8ccccc78)c7ccccc67)c45)c4ccccc34)cccc2c1 ; c1ccc2c(-c3ccc(-c4c5ccccc5c(-c5cccc6oc7cc(-c8c9ccccc9c(-c9cccc%10ccccc9%10)c9ccccc89)ccc7c56)c5ccccc45)cc3)cccc2c1 ; c1ccc(-c2ccc(-c3c4ccccc4c(-c4ccc5c(c4)oc4cccc(-c6c7ccccc7c(-c7ccc(-c8ccccc8)c8ccccc78)c7ccccc67)c45)c4ccccc34)c3ccccc23)cc1]

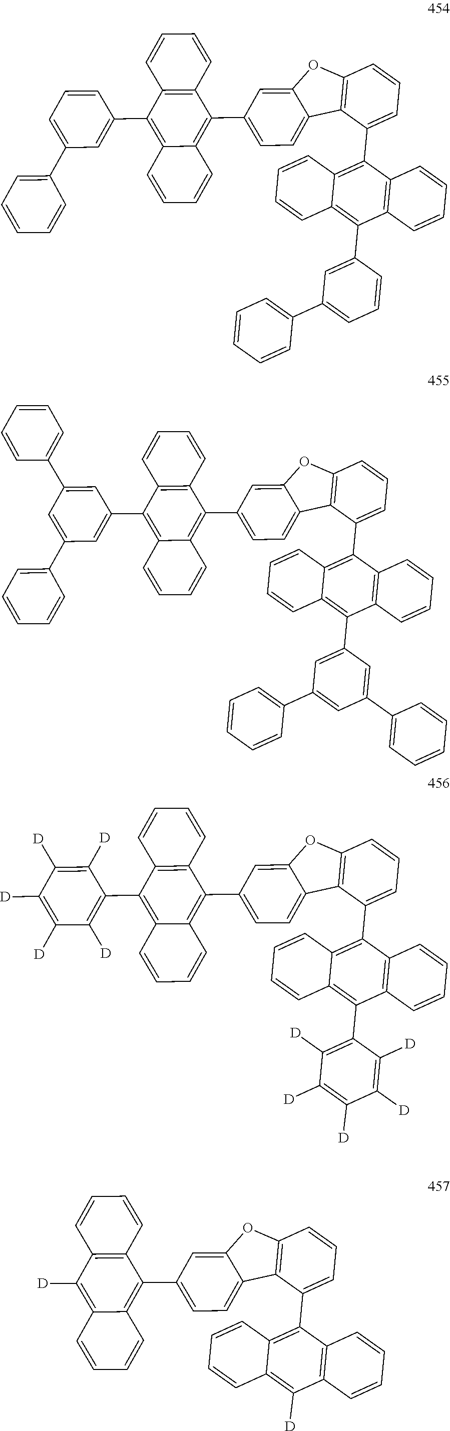 [SMILES: c1ccc(-c2cccc(-c3c4ccccc4c(-c4ccc5c(c4)oc4cccc(-c6c7ccccc7c(-c7cccc(-c8ccccc8)c7)c7ccccc67)c45)c4ccccc34)c2)cc1 ; c1ccc(-c2cc(-c3ccccc3)cc(-c3c4ccccc4c(-c4ccc5c(c4)oc4cccc(-c6c7ccccc7c(-c7cc(-c8ccccc8)cc(-c8ccccc8)c7)c7ccccc67)c45)c4ccccc34)c2)cc1 ; [2H]c1c([2H])c([2H])c(-c2c3ccccc3c(-c3ccc4c(c3)oc3cccc(-c5c6ccccc6c(-c6c([2H])c([2H])c([2H])c([2H])c6[2H])c6ccccc56)c34)c3ccccc23)c([2H])c1[2H] ; [2H]c1c2ccccc2c(-c2ccc3c(c2)oc2cccc(-c4c5ccccc5c([2H])c5ccccc45)c23)c2ccccc12]

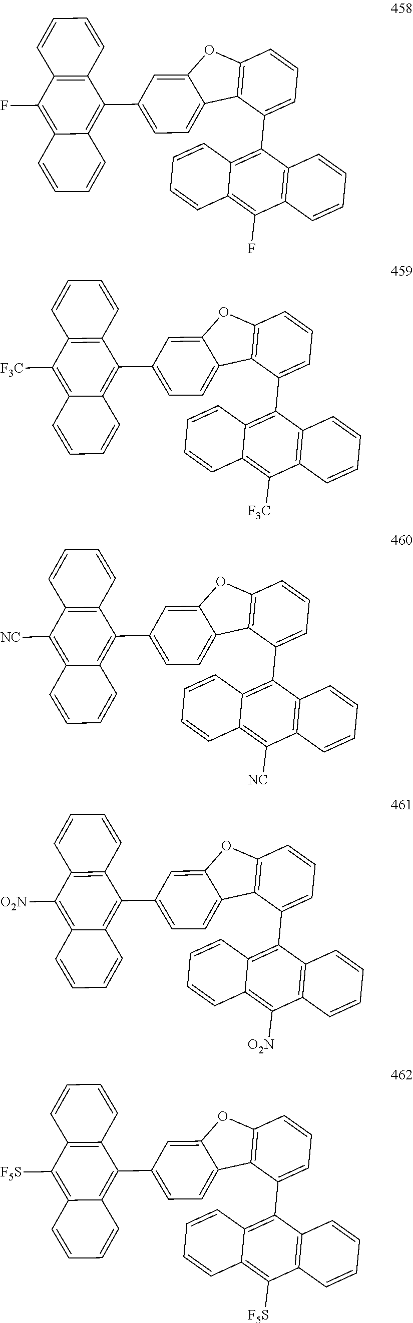

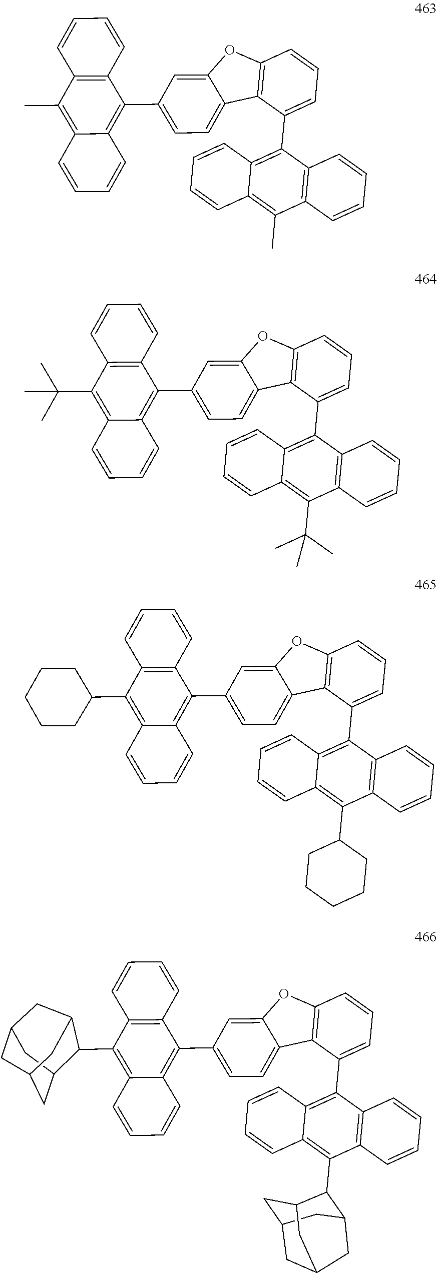 [SMILES: Cc1c2ccccc2c(-c2ccc3c(c2)oc2cccc(-c4c5ccccc5c(C)c5ccccc45)c23)c2ccccc12 ; CC(C)(C)c1c2ccccc2c(-c2ccc3c(c2)oc2cccc(-c4c5ccccc5c(C(C)(C)C)c5ccccc45)c23)c2ccccc12 ; c1cc(-c2c3ccccc3c(C3CCCCC3)c3ccccc23)c2c(c1)oc1cc(-c3c4ccccc4c(C4CCCCC4)c4ccccc34)ccc12 ; c1cc(-c2c3ccccc3c(C3C4CC5CC(C4)CC3C5)c3ccccc23)c2c(c1)oc1cc(-c3c4ccccc4c(C4C5CC6CC(C5)CC4C6)c4ccccc34)ccc12]

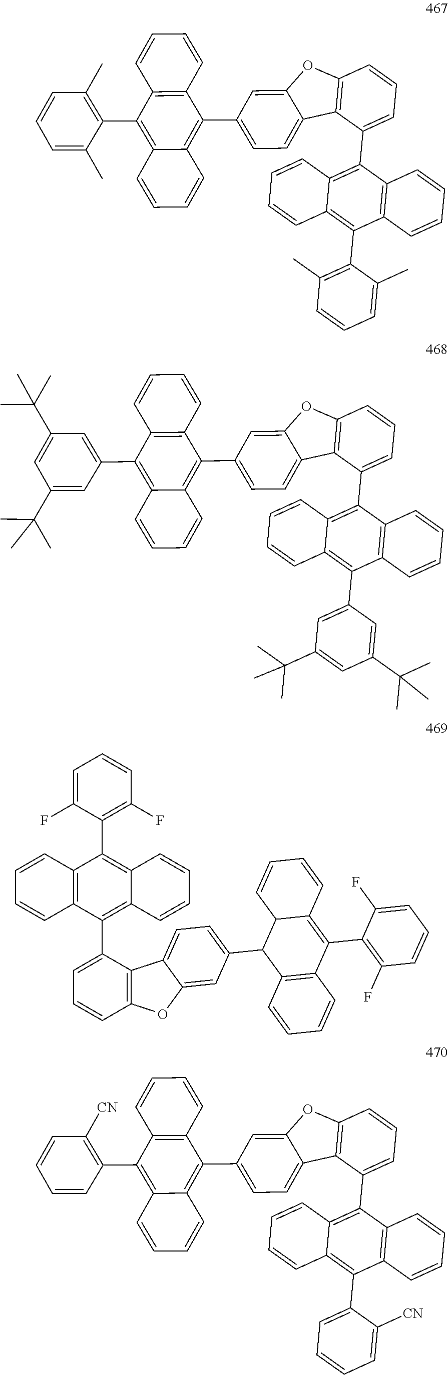 [SMILES: Cc1cccc(C)c1-c1c2ccccc2c(-c2ccc3c(c2)oc2cccc(-c4c5ccccc5c(-c5c(C)cccc5C)c5ccccc45)c23)c2ccccc12 ; CC(C)(C)c1cc(-c2c3ccccc3c(-c3ccc4c(c3)oc3cccc(-c5c6ccccc6c(-c6cc(C(C)(C)C)cc(C(C)(C)C)c6)c6ccccc56)c34)c3ccccc23)cc(C(C)(C)C)c1 ; Fc1cccc(F)c1C1=C2C=CC=CC2C(c2ccc3c(c2)oc2cccc(-c4c5ccccc5c(-c5c(F)cccc5F)c5ccccc45)c23)c2ccccc21 ; N#Cc1ccccc1-c1c2ccccc2c(-c2ccc3c(c2)oc2cccc(-c4c5ccccc5c(-c5ccccc5C#N)c5ccccc45)c23)c2ccccc12]

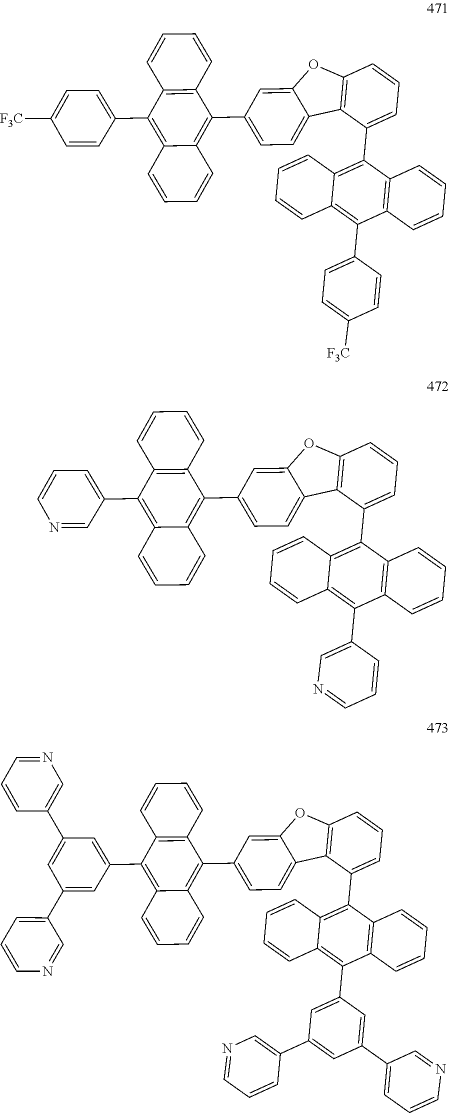 [SMILES: FC(F)(F)c1ccc(-c2c3ccccc3c(-c3ccc4c(c3)oc3cccc(-c5c6ccccc6c(-c6ccc(C(F)(F)F)cc6)c6ccccc56)c34)c3ccccc23)cc1 ; c1cncc(-c2c3ccccc3c(-c3ccc4c(c3)oc3cccc(-c5c6ccccc6c(-c6cccnc6)c6ccccc56)c34)c3ccccc23)c1 ; c1cncc(-c2cc(-c3cccnc3)cc(-c3c4ccccc4c(-c4ccc5c(c4)oc4cccc(-c6c7ccccc7c(-c7cc(-c8cccnc8)cc(-c8cccnc8)c7)c7ccccc67)c45)c4ccccc34)c2)c1]

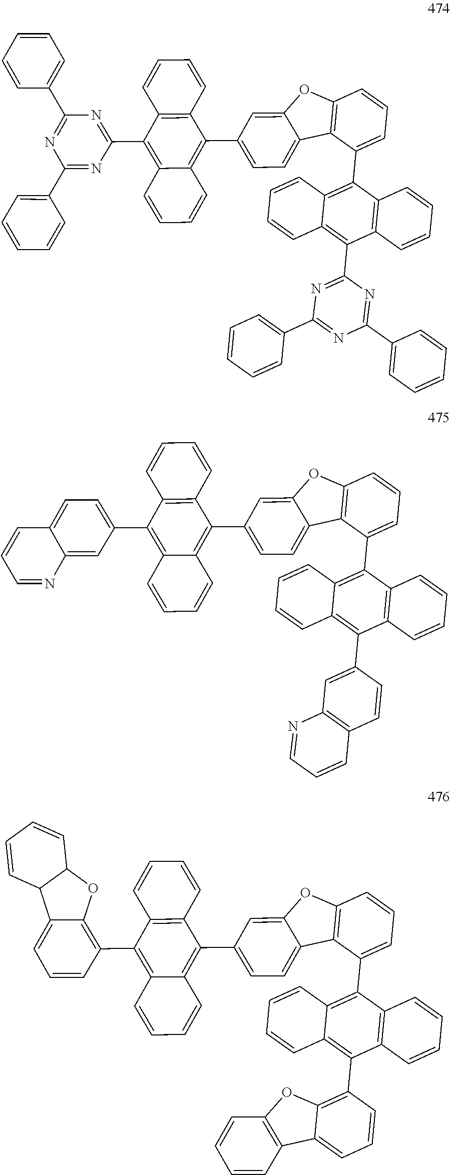 [SMILES: c1ccc(-c2nc(-c3ccccc3)nc(-c3c4ccccc4c(-c4ccc5c(c4)oc4cccc(-c6c7ccccc7c(-c7nc(-c8ccccc8)nc(-c8ccccc8)n7)c7ccccc67)c45)c4ccccc34)n2)cc1 ; c1cnc2cc(-c3c4ccccc4c(-c4ccc5c(c4)oc4cccc(-c6c7ccccc7c(-c7ccc8cccnc8c7)c7ccccc67)c45)c4ccccc34)ccc2c1 ; C1=CC2Oc3c(-c4c5ccccc5c(-c5ccc6c(c5)oc5cccc(-c7c8ccccc8c(-c8cccc9c8oc8ccccc89)c8ccccc78)c56)c5ccccc45)cccc3C2C=C1]

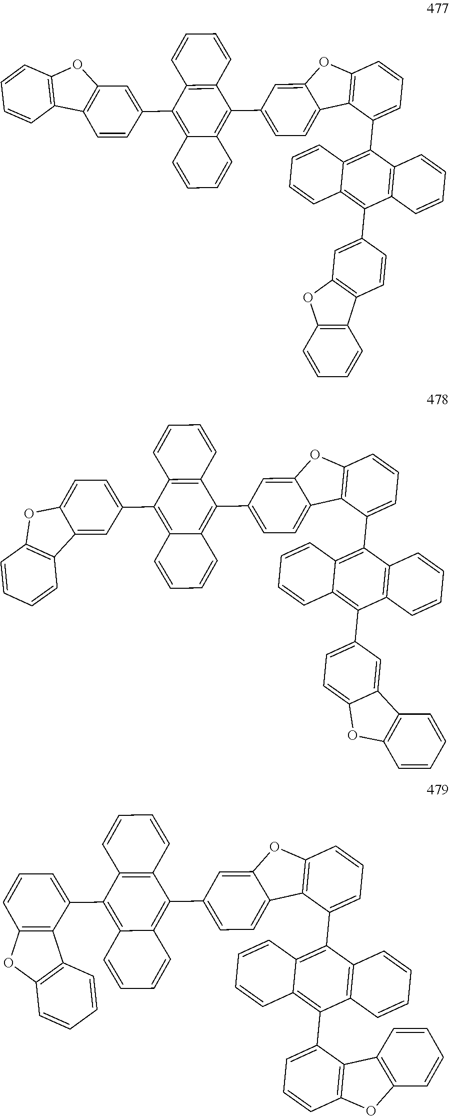 [SMILES: c1ccc2c(c1)oc1cc(-c3c4ccccc4c(-c4ccc5c(c4)oc4cccc(-c6c7ccccc7c(-c7ccc8c(c7)oc7ccccc78)c7ccccc67)c45)c4ccccc34)ccc12 ; c1ccc2c(c1)oc1ccc(-c3c4ccccc4c(-c4ccc5c(c4)oc4cccc(-c6c7ccccc7c(-c7ccc8oc9ccccc9c8c7)c7ccccc67)c45)c4ccccc34)cc12 ; c1ccc2c(c1)oc1cccc(-c3c4ccccc4c(-c4ccc5c(c4)oc4cccc(-c6c7ccccc7c(-c7cccc8oc9ccccc9c78)c7ccccc67)c45)c4ccccc34)c12]

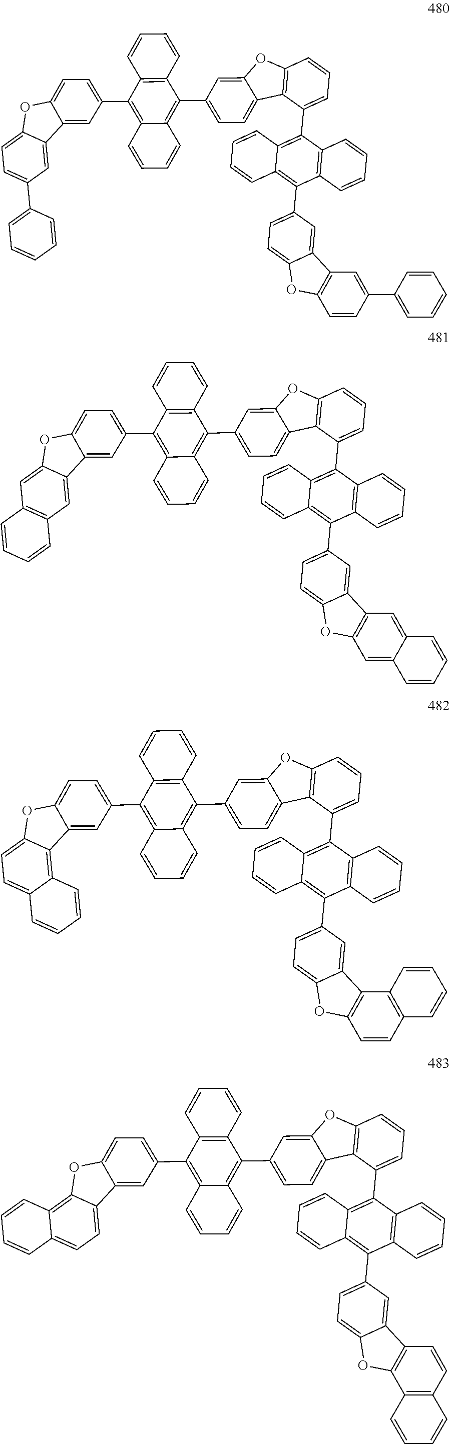

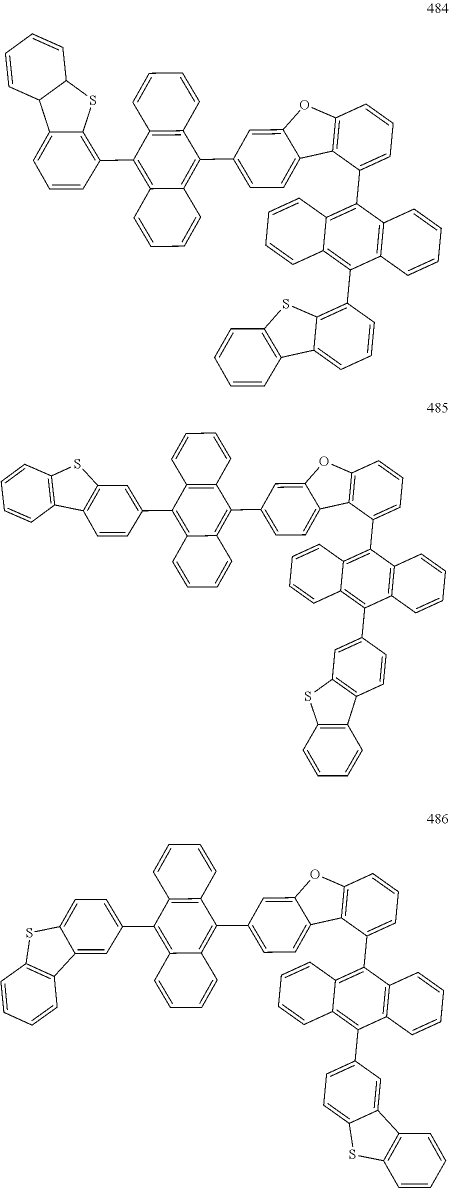 [SMILES: C1=CC2Sc3c(-c4c5ccccc5c(-c5ccc6c(c5)oc5cccc(-c7c8ccccc8c(-c8cccc9c8sc8ccccc89)c8ccccc78)c56)c5ccccc45)cccc3C2C=C1 ; c1cc(-c2c3ccccc3c(-c3ccc4c(c3)sc3ccccc34)c3ccccc23)c2c(c1)oc1cc(-c3c4ccccc4c(-c4ccc5c(c4)sc4ccccc45)c4ccccc34)ccc12 ; c1cc(-c2c3ccccc3c(-c3ccc4sc5ccccc5c4c3)c3ccccc23)c2c(c1)oc1cc(-c3c4ccccc4c(-c4ccc5sc6ccccc6c5c4)c4ccccc34)ccc12]

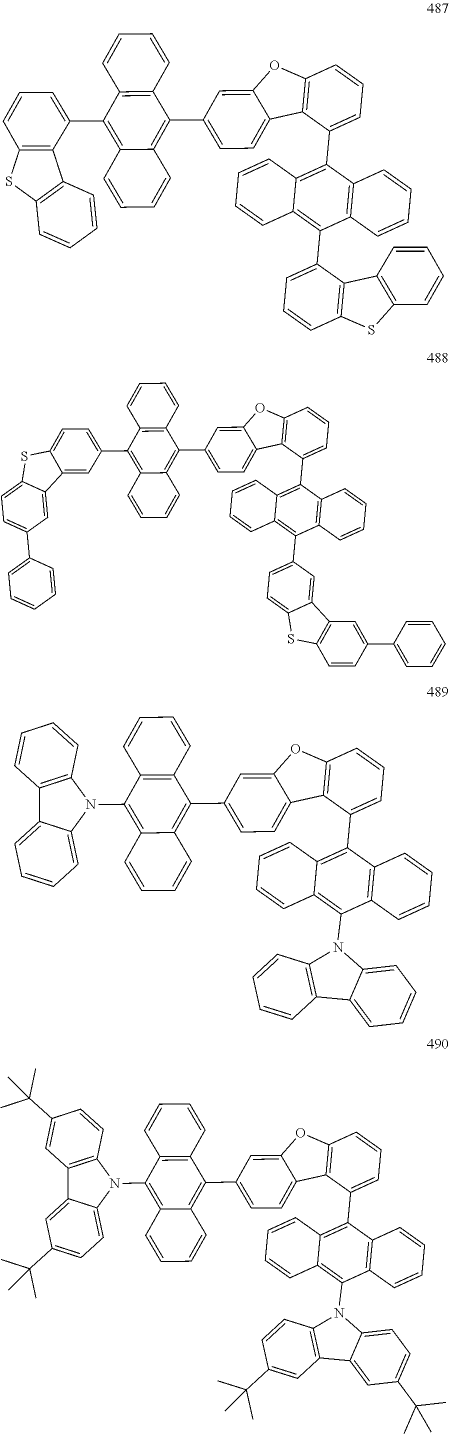

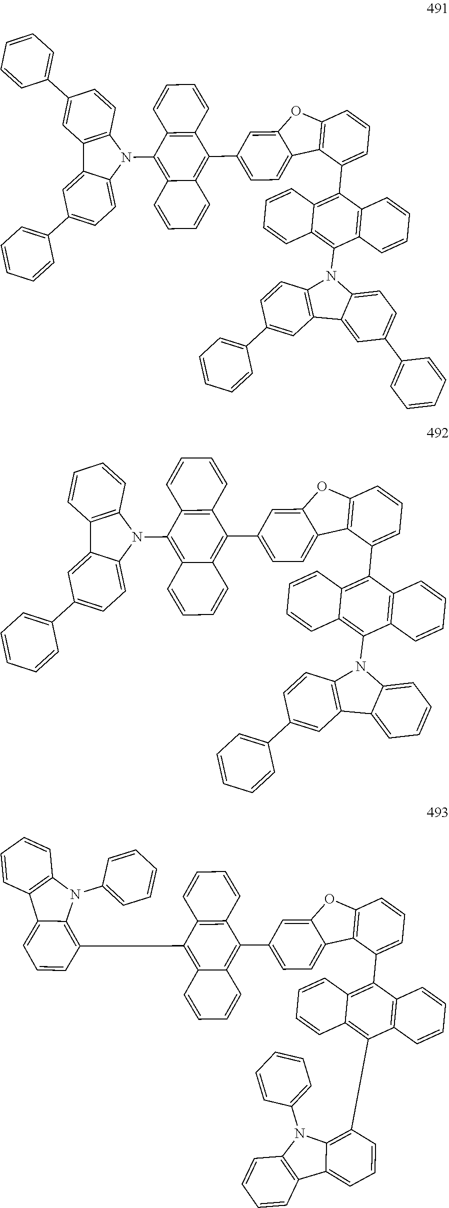 [SMILES: c1ccc(-c2ccc3c(c2)c2cc(-c4ccccc4)ccc2n3-c2c3ccccc3c(-c3ccc4c(c3)oc3cccc(-c5c6ccccc6c(-n6c7ccc(-c8ccccc8)cc7c7cc(-c8ccccc8)ccc76)c6ccccc56)c34)c3ccccc23)cc1 ; c1ccc(-c2ccc3c(c2)c2ccccc2n3-c2c3ccccc3c(-c3ccc4c(c3)oc3cccc(-c5c6ccccc6c(-n6c7ccccc7c7cc(-c8ccccc8)ccc76)c6ccccc56)c34)c3ccccc23)cc1 ; c1ccc(-n2c3ccccc3c3cccc(-c4c5ccccc5c(-c5ccc6c(c5)oc5cccc(-c7c8ccccc8c(-c8cccc9c%10ccccc%10n(-c%10ccccc%10)c89)c8ccccc78)c56)c5ccccc45)c32)cc1]

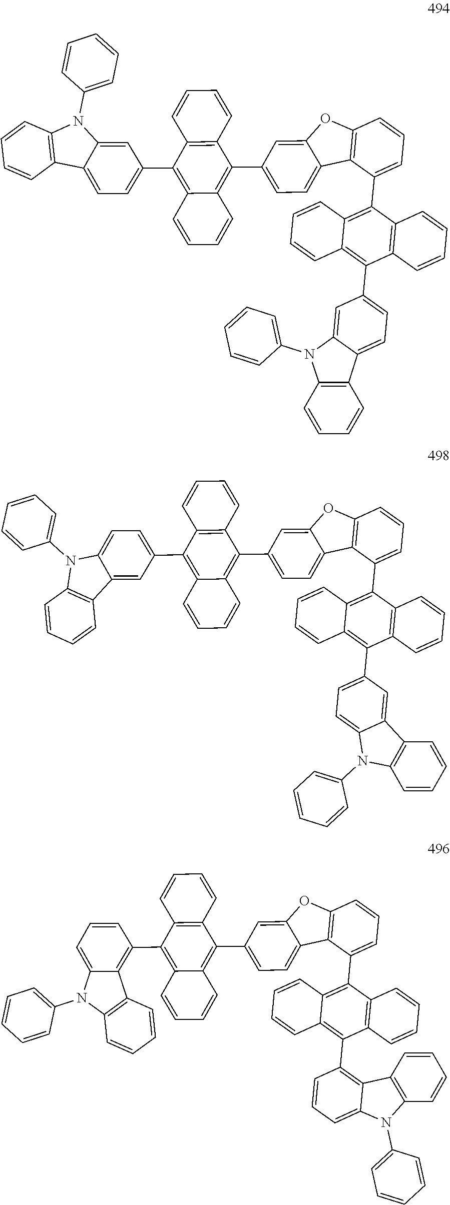 [SMILES: c1ccc(-n2c3ccccc3c3ccc(-c4c5ccccc5c(-c5ccc6c(c5)oc5cccc(-c7c8ccccc8c(-c8ccc9c%10ccccc%10n(-c%10ccccc%10)c9c8)c8ccccc78)c56)c5ccccc45)cc32)cc1 ; c1ccc(-n2c3ccccc3c3cc(-c4c5ccccc5c(-c5ccc6c(c5)oc5cccc(-c7c8ccccc8c(-c8ccc9c(c8)c8ccccc8n9-c8ccccc8)c8ccccc78)c56)c5ccccc45)ccc32)cc1 ; c1ccc(-n2c3ccccc3c3c(-c4c5ccccc5c(-c5ccc6c(c5)oc5cccc(-c7c8ccccc8c(-c8cccc9c8c8ccccc8n9-c8ccccc8)c8ccccc78)c56)c5ccccc45)cccc32)cc1]

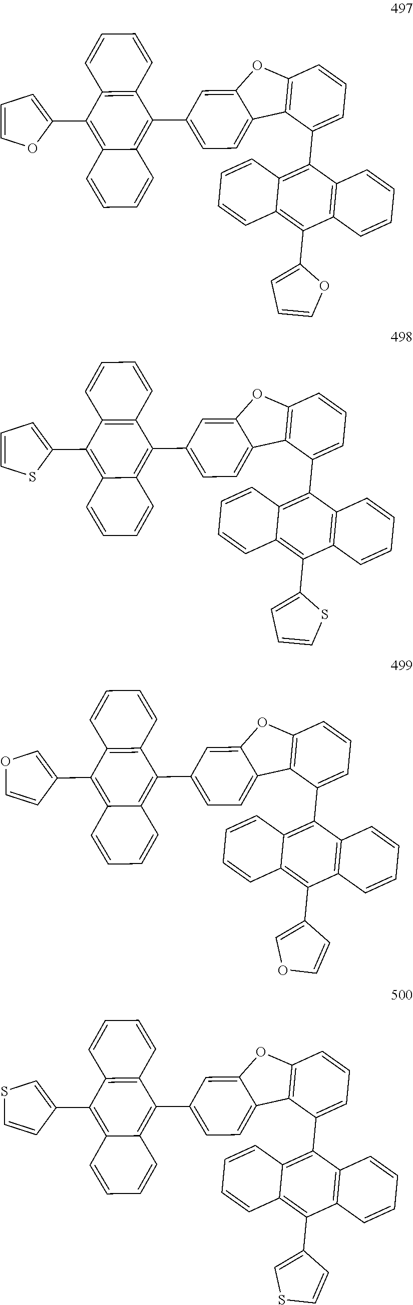 [SMILES: c1coc(-c2c3ccccc3c(-c3ccc4c(c3)oc3cccc(-c5c6ccccc6c(-c6ccco6)c6ccccc56)c34)c3ccccc23)c1 ; c1csc(-c2c3ccccc3c(-c3ccc4c(c3)oc3cccc(-c5c6ccccc6c(-c6cccs6)c6ccccc56)c34)c3ccccc23)c1 ; c1cc(-c2c3ccccc3c(-c3ccoc3)c3ccccc23)c2c(c1)oc1cc(-c3c4ccccc4c(-c4ccoc4)c4ccccc34)ccc12 ; c1cc(-c2c3ccccc3c(-c3ccsc3)c3ccccc23)c2c(c1)oc1cc(-c3c4ccccc4c(-c4ccsc4)c4ccccc34)ccc12]

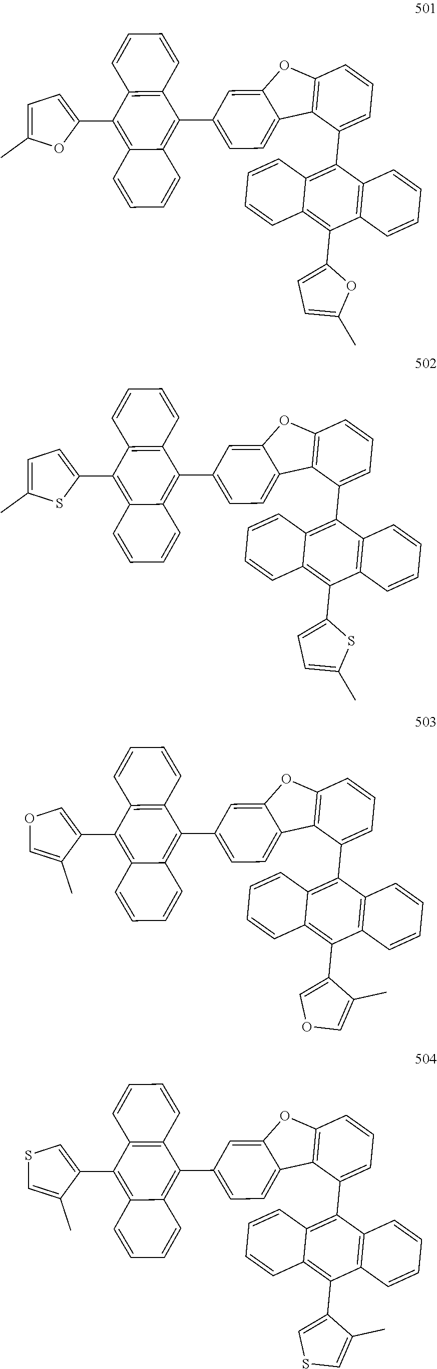 [SMILES: Cc1ccc(-c2c3ccccc3c(-c3ccc4c(c3)oc3cccc(-c5c6ccccc6c(-c6ccc(C)o6)c6ccccc56)c34)c3ccccc23)o1 ; Cc1ccc(-c2c3ccccc3c(-c3ccc4c(c3)oc3cccc(-c5c6ccccc6c(-c6ccc(C)s6)c6ccccc56)c34)c3ccccc23)s1 ; Cc1cocc1-c1c2ccccc2c(-c2ccc3c(c2)oc2cccc(-c4c5ccccc5c(-c5cocc5C)c5ccccc45)c23)c2ccccc12 ; Cc1cscc1-c1c2ccccc2c(-c2ccc3c(c2)oc2cccc(-c4c5ccccc5c(-c5cscc5C)c5ccccc45)c23)c2ccccc12]

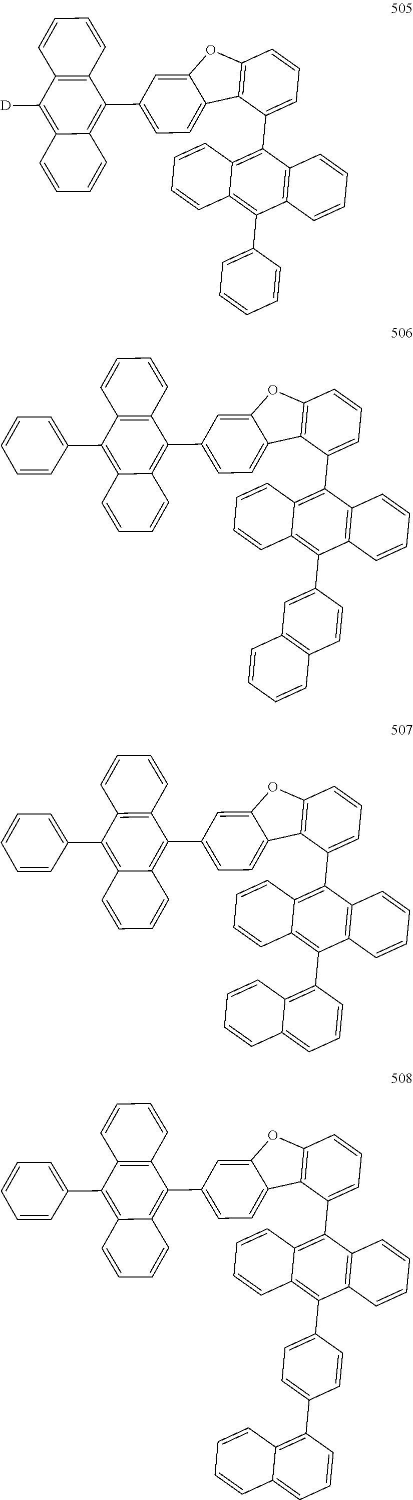 [SMILES: [2H]c1c2ccccc2c(-c2ccc3c(c2)oc2cccc(-c4c5ccccc5c(-c5ccccc5)c5ccccc45)c23)c2ccccc12 ; c1ccc(-c2c3ccccc3c(-c3ccc4c(c3)oc3cccc(-c5c6ccccc6c(-c6ccc7ccccc7c6)c6ccccc56)c34)c3ccccc23)cc1 ; c1ccc(-c2c3ccccc3c(-c3ccc4c(c3)oc3cccc(-c5c6ccccc6c(-c6cccc7ccccc67)c6ccccc56)c34)c3ccccc23)cc1 ; c1ccc(-c2c3ccccc3c(-c3ccc4c(c3)oc3cccc(-c5c6ccccc6c(-c6ccc(-c7cccc8ccccc78)cc6)c6ccccc56)c34)c3ccccc23)cc1]

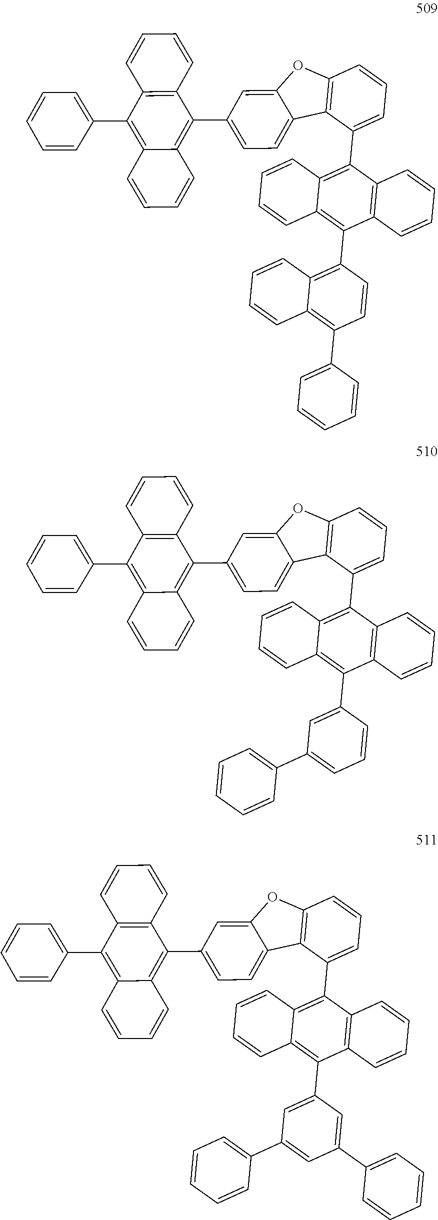 [SMILES: c1ccc(-c2ccc(-c3c4ccccc4c(-c4cccc5oc6cc(-c7c8ccccc8c(-c8ccccc8)c8ccccc78)ccc6c45)c4ccccc34)c3ccccc23)cc1 ; c1ccc(-c2cccc(-c3c4ccccc4c(-c4cccc5oc6cc(-c7c8ccccc8c(-c8ccccc8)c8ccccc78)ccc6c45)c4ccccc34)c2)cc1 ; c1ccc(-c2cc(-c3ccccc3)cc(-c3c4ccccc4c(-c4cccc5oc6cc(-c7c8ccccc8c(-c8ccccc8)c8ccccc78)ccc6c45)c4ccccc34)c2)cc1]

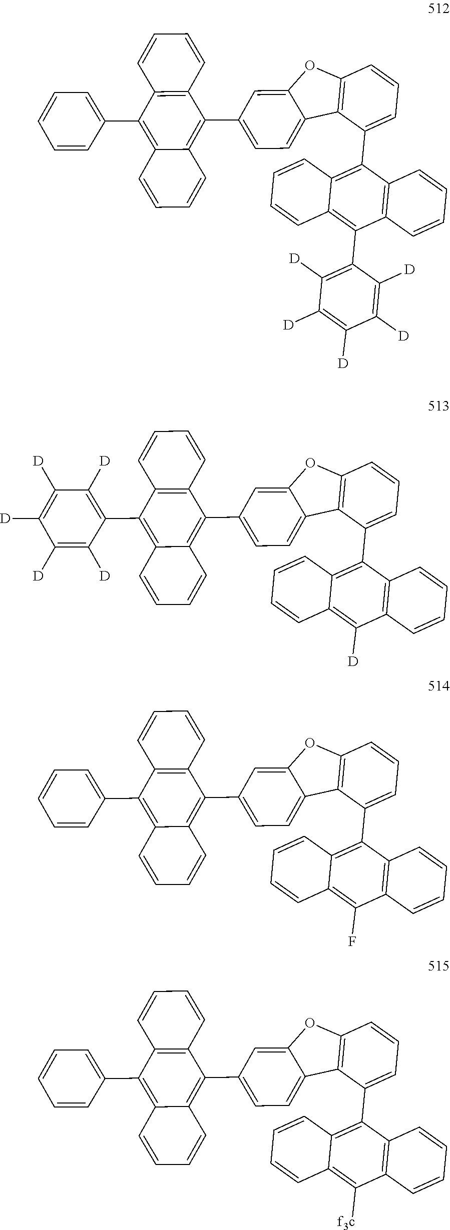

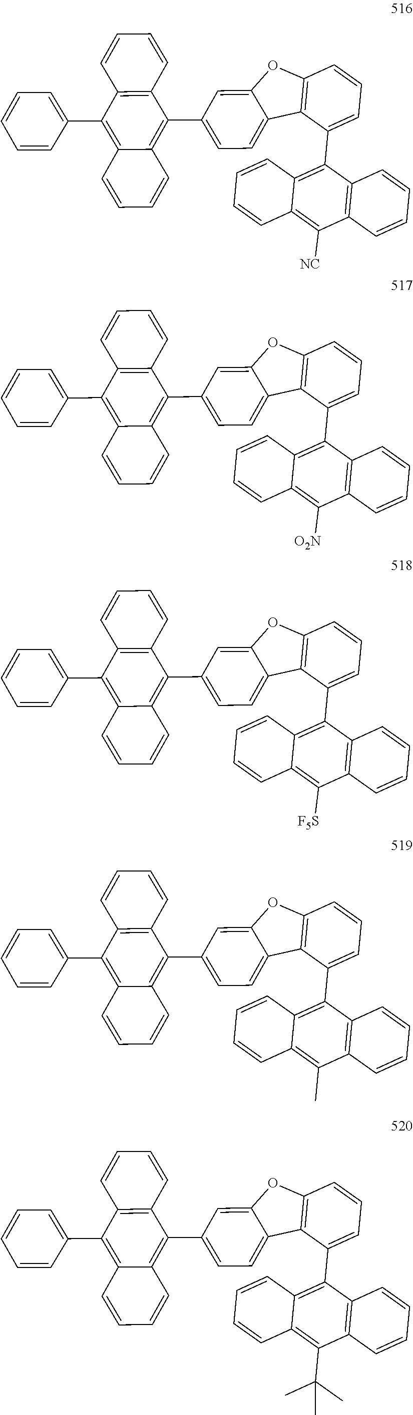

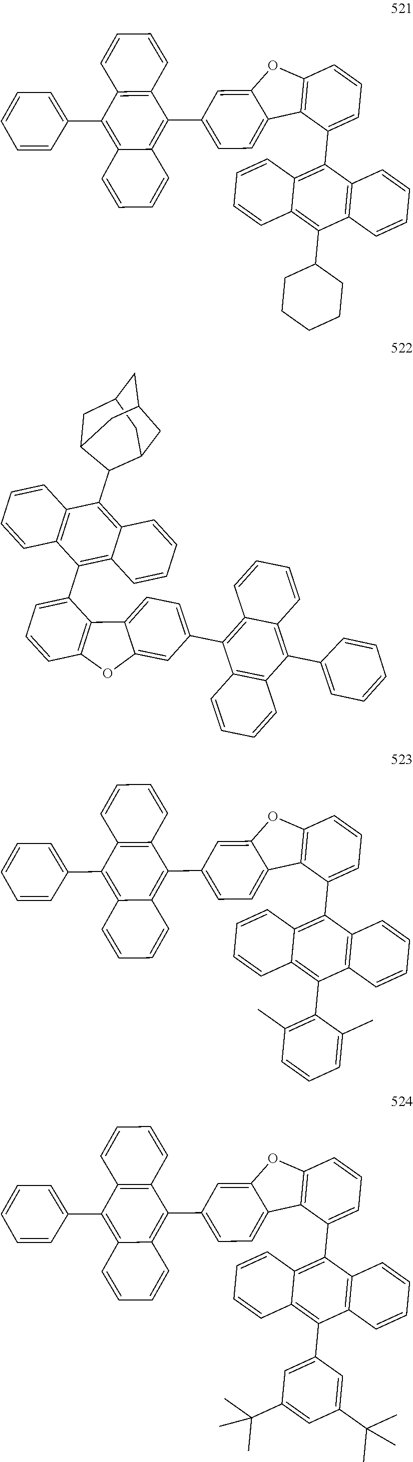 [SMILES: c1ccc(-c2c3ccccc3c(-c3ccc4c(c3)oc3cccc(-c5c6ccccc6c(C6CCCCC6)c6ccccc56)c34)c3ccccc23)cc1 ; c1ccc(-c2c3ccccc3c(-c3ccc4c(c3)oc3cccc(-c5c6ccccc6c(C6C7CC8CC(C7)CC6C8)c6ccccc56)c34)c3ccccc23)cc1 ; Cc1cccc(C)c1-c1c2ccccc2c(-c2cccc3oc4cc(-c5c6ccccc6c(-c6ccccc6)c6ccccc56)ccc4c23)c2ccccc12 ; CC(C)(C)c1cc(-c2c3ccccc3c(-c3cccc4oc5cc(-c6c7ccccc7c(-c7ccccc7)c7ccccc67)ccc5c34)c3ccccc23)cc(C(C)(C)C)c1]

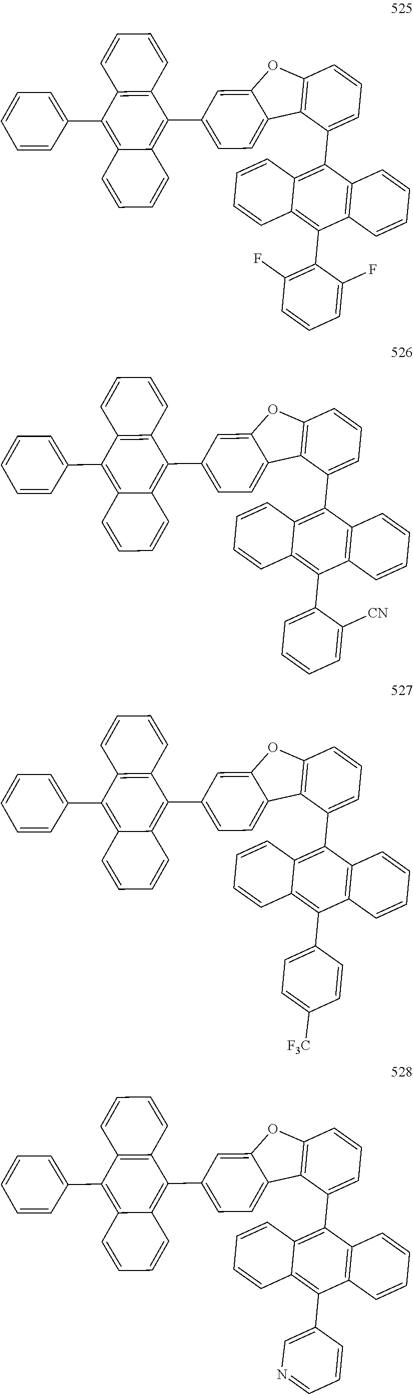 [SMILES: Fc1cccc(F)c1-c1c2ccccc2c(-c2cccc3oc4cc(-c5c6ccccc6c(-c6ccccc6)c6ccccc56)ccc4c23)c2ccccc12 ; N#Cc1ccccc1-c1c2ccccc2c(-c2cccc3oc4cc(-c5c6ccccc6c(-c6ccccc6)c6ccccc56)ccc4c23)c2ccccc12 ; FC(F)(F)c1ccc(-c2c3ccccc3c(-c3cccc4oc5cc(-c6c7ccccc7c(-c7ccccc7)c7ccccc67)ccc5c34)c3ccccc23)cc1 ; c1ccc(-c2c3ccccc3c(-c3ccc4c(c3)oc3cccc(-c5c6ccccc6c(-c6cccnc6)c6ccccc56)c34)c3ccccc23)cc1]

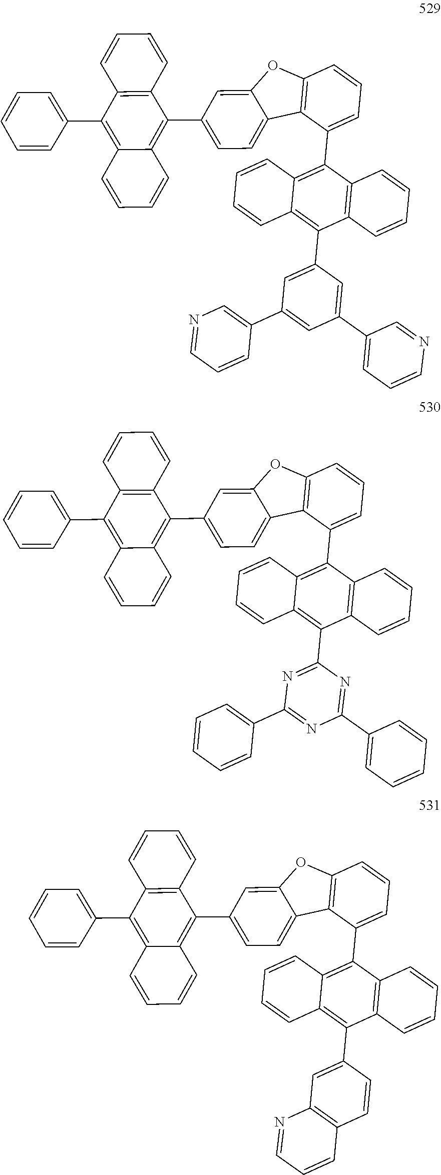 [SMILES: c1ccc(-c2c3ccccc3c(-c3ccc4c(c3)oc3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5oc6cc(-c7c8ccccc8c(-c8ccccc8)c8ccccc78)ccc6c45)c4ccccc34)n2)cc1 ; c1ccc(-c2c3ccccc3c(-c3ccc4c(c3)oc3cccc(-c5c6ccccc6c(-c6ccc7cccnc7c6)c6ccccc56)c34)c3ccccc23)cc1]

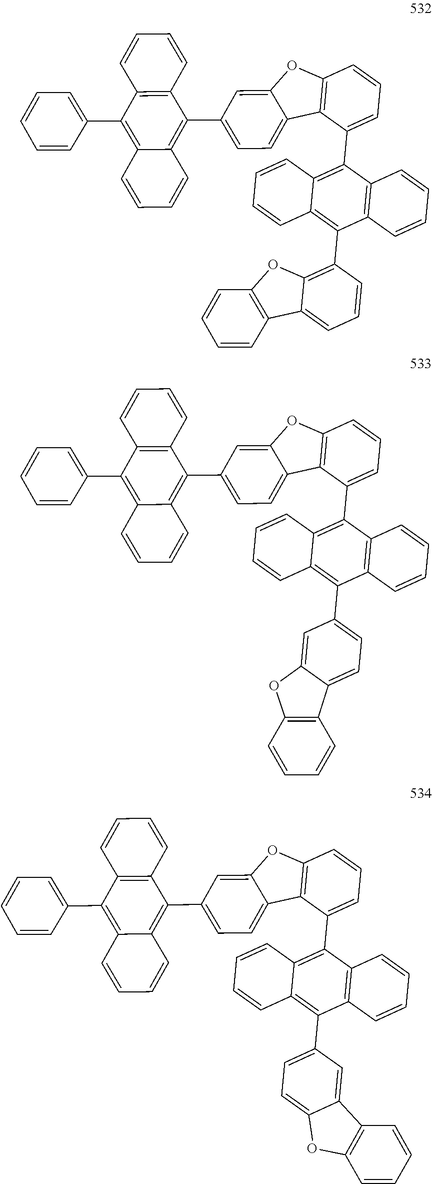 [SMILES: c1ccc(-c2c3ccccc3c(-c3ccc4c(c3)oc3cccc(-c5c6ccccc6c(-c6cccc7c6oc6ccccc67)c6ccccc56)c34)c3ccccc23)cc1 ; c1ccc(-c2c3ccccc3c(-c3ccc4c(c3)oc3cccc(-c5c6ccccc6c(-c6ccc7c(c6)oc6ccccc67)c6ccccc56)c34)c3ccccc23)cc1 ; c1ccc(-c2c3ccccc3c(-c3ccc4c(c3)oc3cccc(-c5c6ccccc6c(-c6ccc7oc8ccccc8c7c6)c6ccccc56)c34)c3ccccc23)cc1]

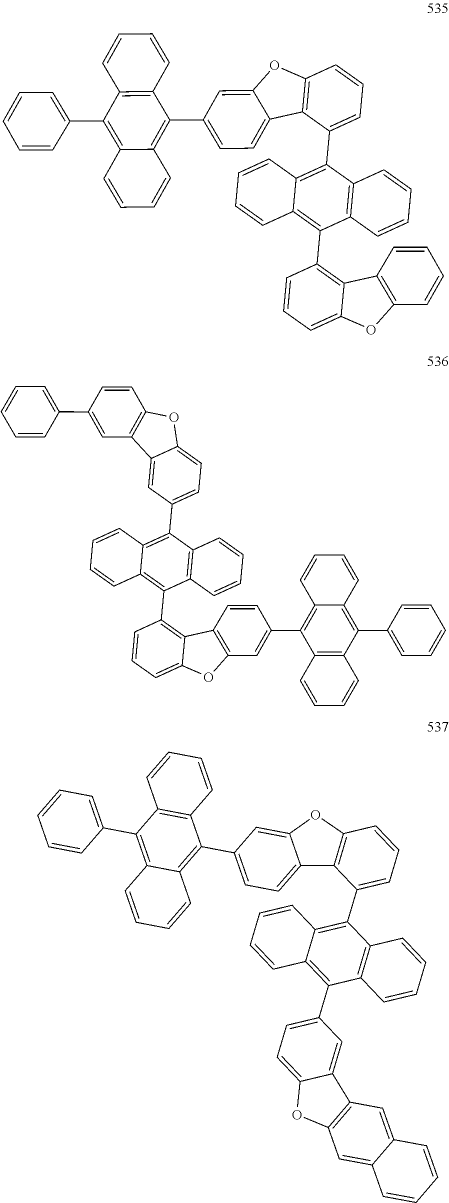 [SMILES: c1ccc(-c2c3ccccc3c(-c3ccc4c(c3)oc3cccc(-c5c6ccccc6c(-c6cccc7oc8ccccc8c67)c6ccccc56)c34)c3ccccc23)cc1 ; c1ccc(-c2ccc3oc4ccc(-c5c6ccccc6c(-c6cccc7oc8cc(-c9c%10ccccc%10c(-c%10ccccc%10)c%10ccccc9%10)ccc8c67)c6ccccc56)cc4c3c2)cc1 ; c1ccc(-c2c3ccccc3c(-c3ccc4c(c3)oc3cccc(-c5c6ccccc6c(-c6ccc7oc8cc9ccccc9cc8c7c6)c6ccccc56)c34)c3ccccc23)cc1]

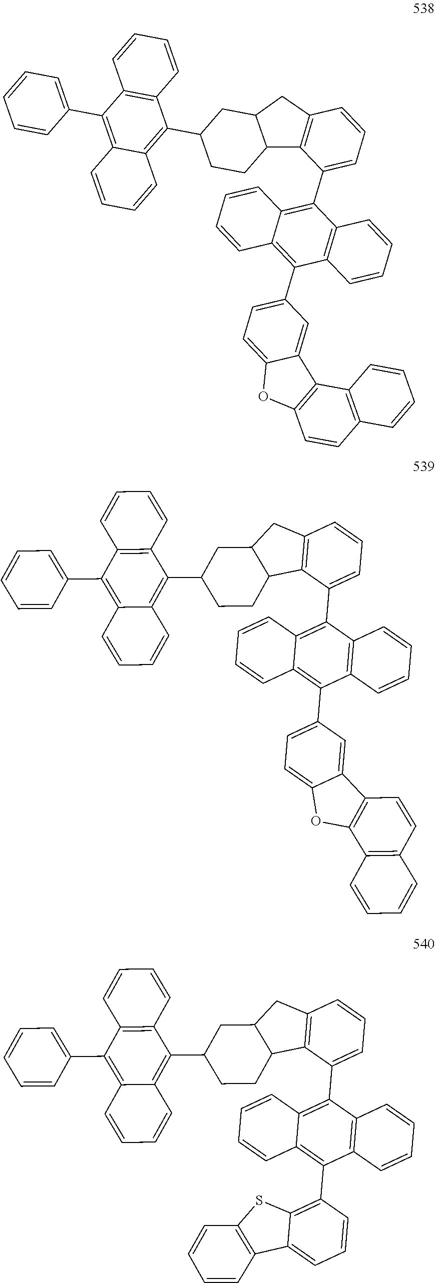 [SMILES: c1ccc(-c2c3ccccc3c(C3CCC4c5c(cccc5-c5c6ccccc6c(-c6ccc7oc8ccc9ccccc9c8c7c6)c6ccccc56)CC4C3)c3ccccc23)cc1 ; c1ccc(-c2c3ccccc3c(C3CCC4c5c(cccc5-c5c6ccccc6c(-c6ccc7oc8c9ccccc9ccc8c7c6)c6ccccc56)CC4C3)c3ccccc23)cc1 ; c1ccc(-c2c3ccccc3c(C3CCC4c5c(cccc5-c5c6ccccc6c(-c6cccc7c6sc6ccccc67)c6ccccc56)CC4C3)c3ccccc23)cc1]

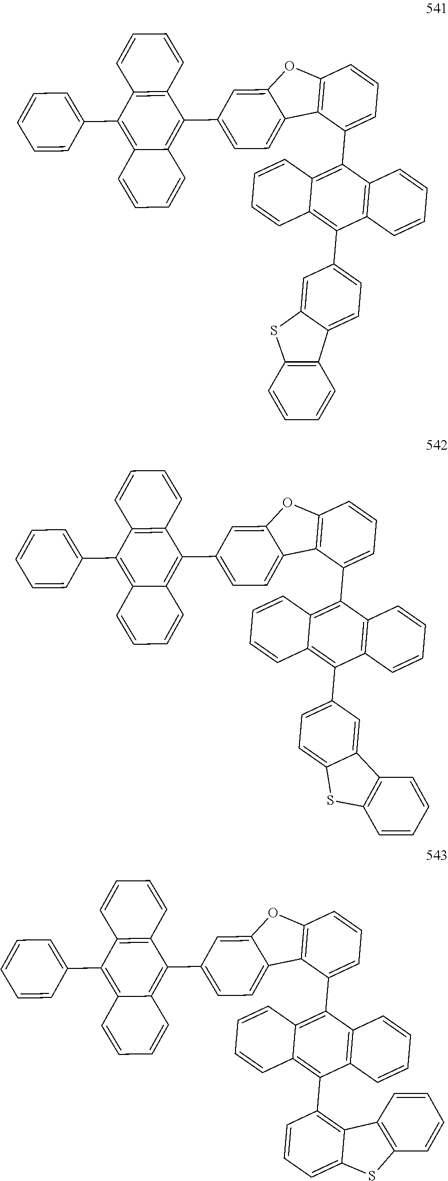 [SMILES: c1ccc(-c2c3ccccc3c(-c3ccc4c(c3)oc3cccc(-c5c6ccccc6c(-c6ccc7c(c6)sc6ccccc67)c6ccccc56)c34)c3ccccc23)cc1 ; c1ccc(-c2c3ccccc3c(-c3ccc4c(c3)oc3cccc(-c5c6ccccc6c(-c6ccc7sc8ccccc8c7c6)c6ccccc56)c34)c3ccccc23)cc1 ; c1ccc(-c2c3ccccc3c(-c3ccc4c(c3)oc3cccc(-c5c6ccccc6c(-c6cccc7sc8ccccc8c67)c6ccccc56)c34)c3ccccc23)cc1]

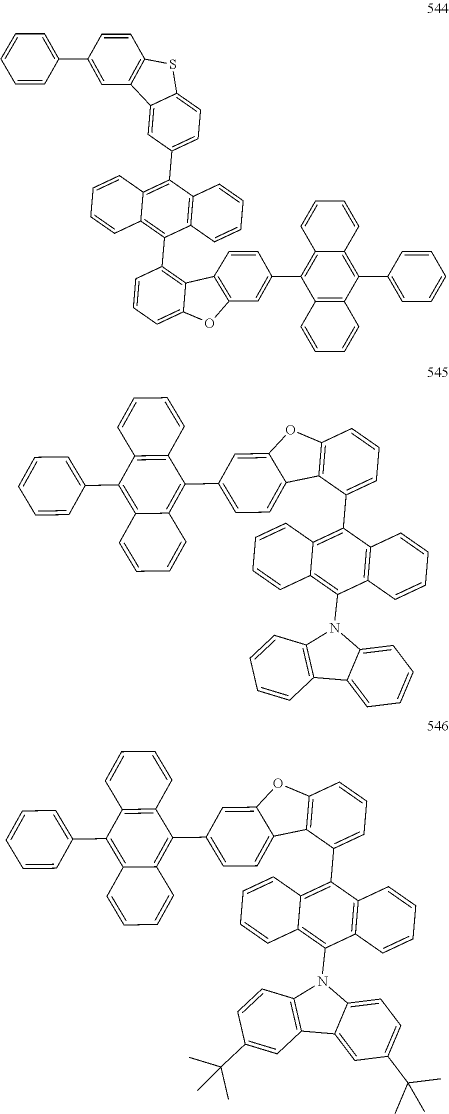 [SMILES: c1ccc(-c2ccc3sc4ccc(-c5c6ccccc6c(-c6cccc7oc8cc(-c9c%10ccccc%10c(-c%10ccccc%10)c%10ccccc9%10)ccc8c67)c6ccccc56)cc4c3c2)cc1 ; c1ccc(-c2c3ccccc3c(-c3ccc4c(c3)oc3cccc(-c5c6ccccc6c(-n6c7ccccc7c7ccccc76)c6ccccc56)c34)c3ccccc23)cc1 ; CC(C)(C)c1ccc2c(c1)c1cc(C(C)(C)C)ccc1n2-c1c2ccccc2c(-c2cccc3oc4cc(-c5c6ccccc6c(-c6ccccc6)c6ccccc56)ccc4c23)c2ccccc12]

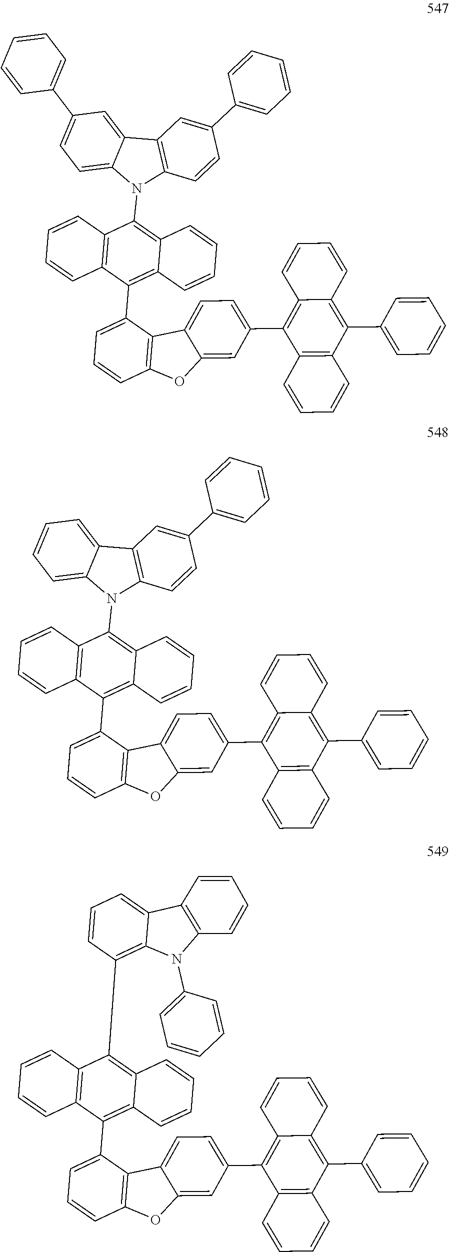 [SMILES: c1ccc(-c2ccc3c(c2)c2cc(-c4ccccc4)ccc2n3-c2c3ccccc3c(-c3cccc4oc5cc(-c6c7ccccc7c(-c7ccccc7)c7ccccc67)ccc5c34)c3ccccc23)cc1 ; c1ccc(-c2ccc3c(c2)c2ccccc2n3-c2c3ccccc3c(-c3cccc4oc5cc(-c6c7ccccc7c(-c7ccccc7)c7ccccc67)ccc5c34)c3ccccc23)cc1 ; c1ccc(-c2c3ccccc3c(-c3ccc4c(c3)oc3cccc(-c5c6ccccc6c(-c6cccc7c8ccccc8n(-c8ccccc8)c67)c6ccccc56)c34)c3ccccc23)cc1]

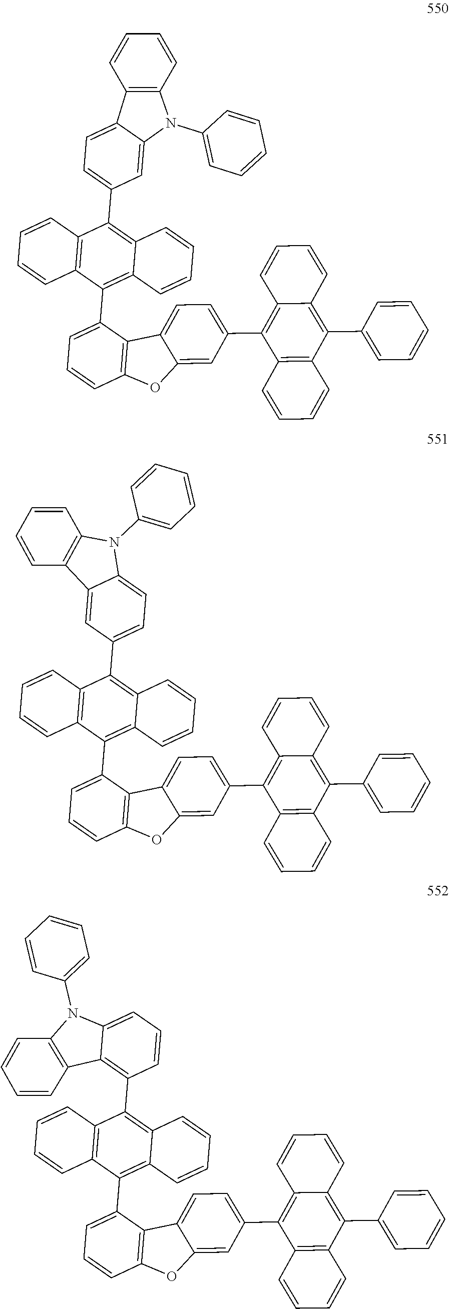 [SMILES: c1ccc(-c2c3ccccc3c(-c3ccc4c(c3)oc3cccc(-c5c6ccccc6c(-c6ccc7c8ccccc8n(-c8ccccc8)c7c6)c6ccccc56)c34)c3ccccc23)cc1 ; c1ccc(-c2c3ccccc3c(-c3ccc4c(c3)oc3cccc(-c5c6ccccc6c(-c6ccc7c(c6)c6ccccc6n7-c6ccccc6)c6ccccc56)c34)c3ccccc23)cc1 ; c1ccc(-c2c3ccccc3c(-c3ccc4c(c3)oc3cccc(-c5c6ccccc6c(-c6cccc7c6c6ccccc6n7-c6ccccc6)c6ccccc56)c34)c3ccccc23)cc1]

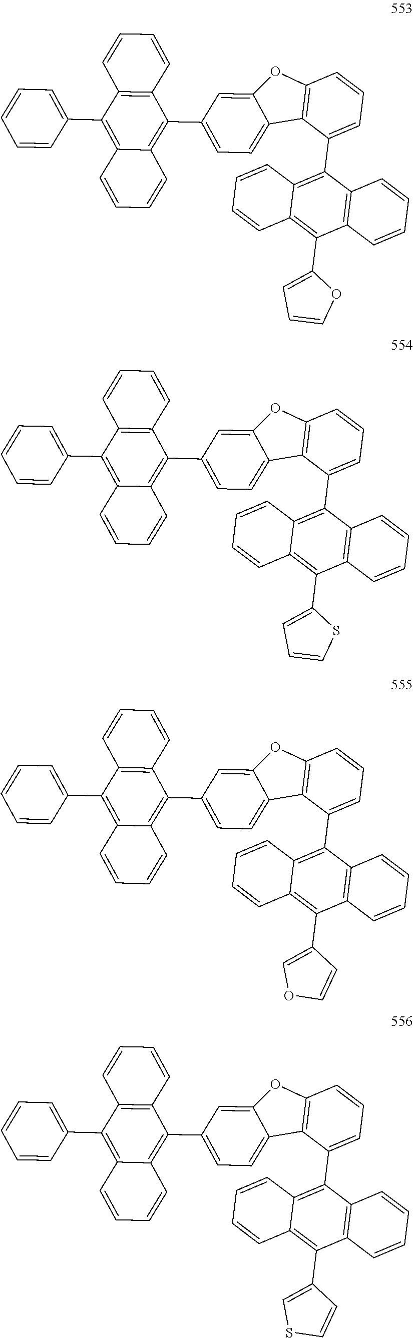 [SMILES: c1ccc(-c2c3ccccc3c(-c3ccc4c(c3)oc3cccc(-c5c6ccccc6c(-c6ccco6)c6ccccc56)c34)c3ccccc23)cc1 ; c1ccc(-c2c3ccccc3c(-c3ccc4c(c3)oc3cccc(-c5c6ccccc6c(-c6cccs6)c6ccccc56)c34)c3ccccc23)cc1 ; c1ccc(-c2c3ccccc3c(-c3ccc4c(c3)oc3cccc(-c5c6ccccc6c(-c6ccoc6)c6ccccc56)c34)c3ccccc23)cc1 ; c1ccc(-c2c3ccccc3c(-c3ccc4c(c3)oc3cccc(-c5c6ccccc6c(-c6ccsc6)c6ccccc56)c34)c3ccccc23)cc1]

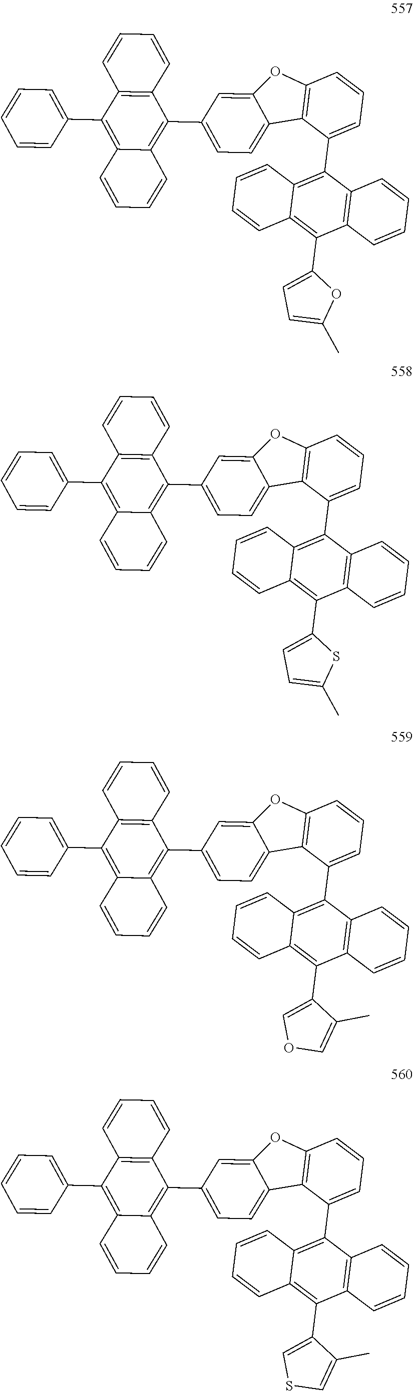 [SMILES: Cc1ccc(-c2c3ccccc3c(-c3cccc4oc5cc(-c6c7ccccc7c(-c7ccccc7)c7ccccc67)ccc5c34)c3ccccc23)o1 ; Cc1ccc(-c2c3ccccc3c(-c3cccc4oc5cc(-c6c7ccccc7c(-c7ccccc7)c7ccccc67)ccc5c34)c3ccccc23)s1 ; Cc1cocc1-c1c2ccccc2c(-c2cccc3oc4cc(-c5c6ccccc6c(-c6ccccc6)c6ccccc56)ccc4c23)c2ccccc12 ; Cc1cscc1-c1c2ccccc2c(-c2cccc3oc4cc(-c5c6ccccc6c(-c6ccccc6)c6ccccc56)ccc4c23)c2ccccc12]

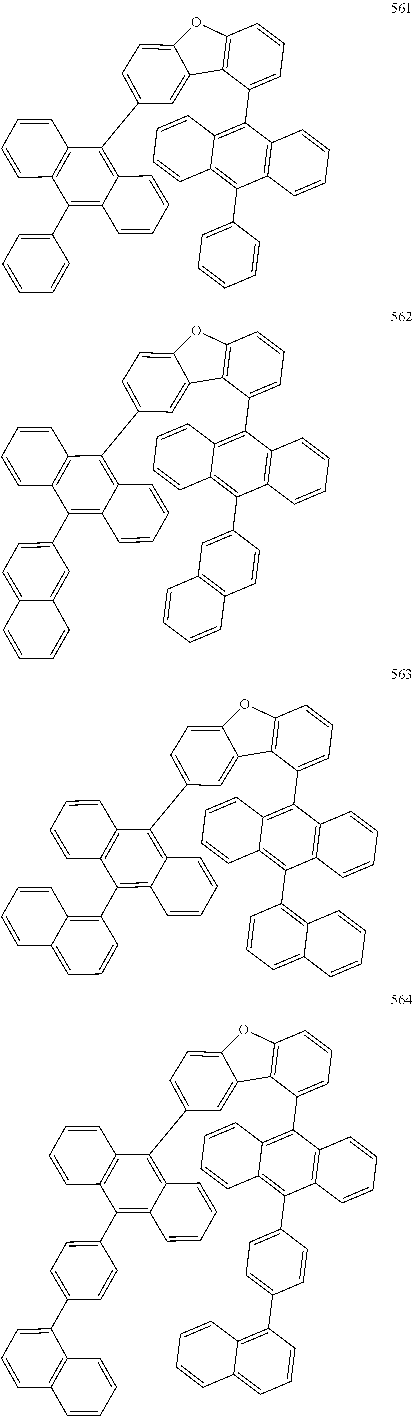 [SMILES: c1ccc(-c2c3ccccc3c(-c3ccc4oc5cccc(-c6c7ccccc7c(-c7ccccc7)c7ccccc67)c5c4c3)c3ccccc23)cc1 ; c1ccc2cc(-c3c4ccccc4c(-c4ccc5oc6cccc(-c7c8ccccc8c(-c8ccc9ccccc9c8)c8ccccc78)c6c5c4)c4ccccc34)ccc2c1 ; c1ccc2c(-c3c4ccccc4c(-c4ccc5oc6cccc(-c7c8ccccc8c(-c8cccc9ccccc89)c8ccccc78)c6c5c4)c4ccccc34)cccc2c1 ; c1ccc2c(-c3ccc(-c4c5ccccc5c(-c5ccc6oc7cccc(-c8c9ccccc9c(-c9ccc(-c%10cccc%11ccccc%10%11)cc9)c9ccccc89)c7c6c5)c5ccccc45)cc3)cccc2c1]

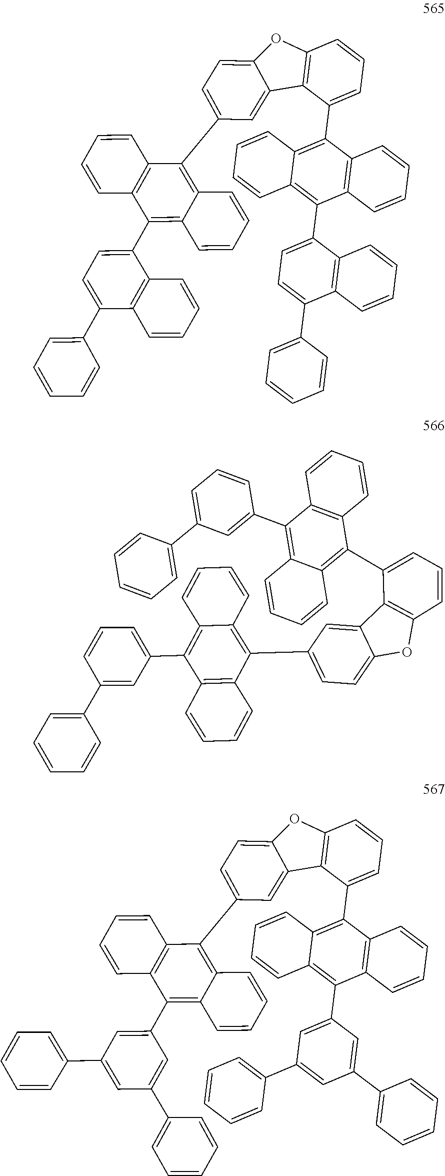 [SMILES: c1ccc(-c2ccc(-c3c4ccccc4c(-c4ccc5oc6cccc(-c7c8ccccc8c(-c8ccc(-c9ccccc9)c9ccccc89)c8ccccc78)c6c5c4)c4ccccc34)c3ccccc23)cc1 ; c1ccc(-c2cccc(-c3c4ccccc4c(-c4ccc5oc6cccc(-c7c8ccccc8c(-c8cccc(-c9ccccc9)c8)c8ccccc78)c6c5c4)c4ccccc34)c2)cc1 ; c1ccc(-c2cc(-c3ccccc3)cc(-c3c4ccccc4c(-c4ccc5oc6cccc(-c7c8ccccc8c(-c8cc(-c9ccccc9)cc(-c9ccccc9)c8)c8ccccc78)c6c5c4)c4ccccc34)c2)cc1]

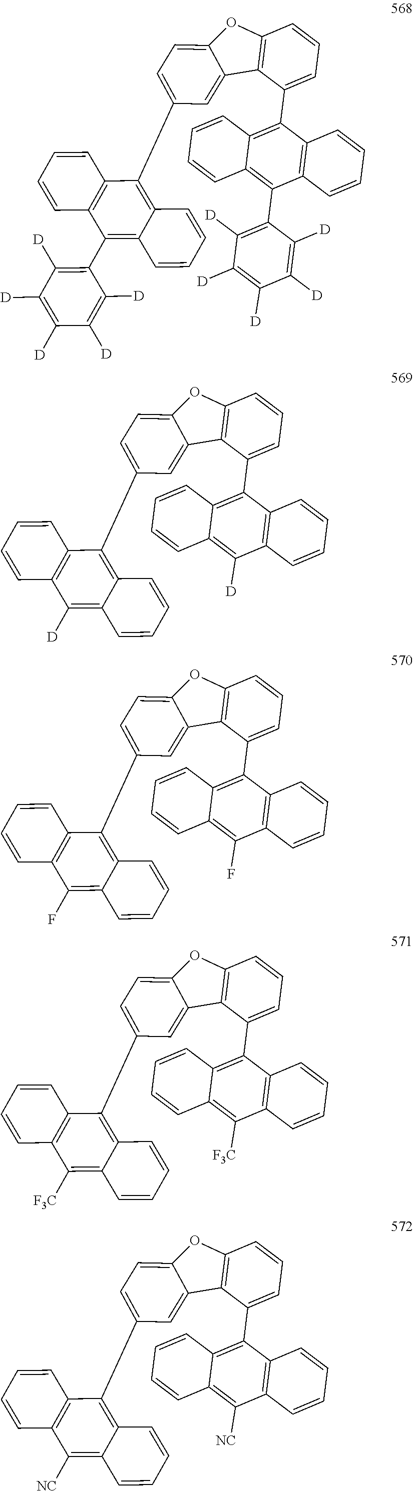 [SMILES: [2H]c1c([2H])c([2H])c(-c2c3ccccc3c(-c3ccc4oc5cccc(-c6c7ccccc7c(-c7c([2H])c([2H])c([2H])c([2H])c7[2H])c7ccccc67)c5c4c3)c3ccccc23)c([2H])c1[2H] ; [2H]c1c2ccccc2c(-c2ccc3oc4cccc(-c5c6ccccc6c([2H])c6ccccc56)c4c3c2)c2ccccc12 ; Fc1c2ccccc2c(-c2ccc3oc4cccc(-c5c6ccccc6c(F)c6ccccc56)c4c3c2)c2ccccc12 ; FC(F)(F)c1c2ccccc2c(-c2ccc3oc4cccc(-c5c6ccccc6c(C(F)(F)F)c6ccccc56)c4c3c2)c2ccccc12 ; N#Cc1c2ccccc2c(-c2ccc3oc4cccc(-c5c6ccccc6c(C#N)c6ccccc56)c4c3c2)c2ccccc12]

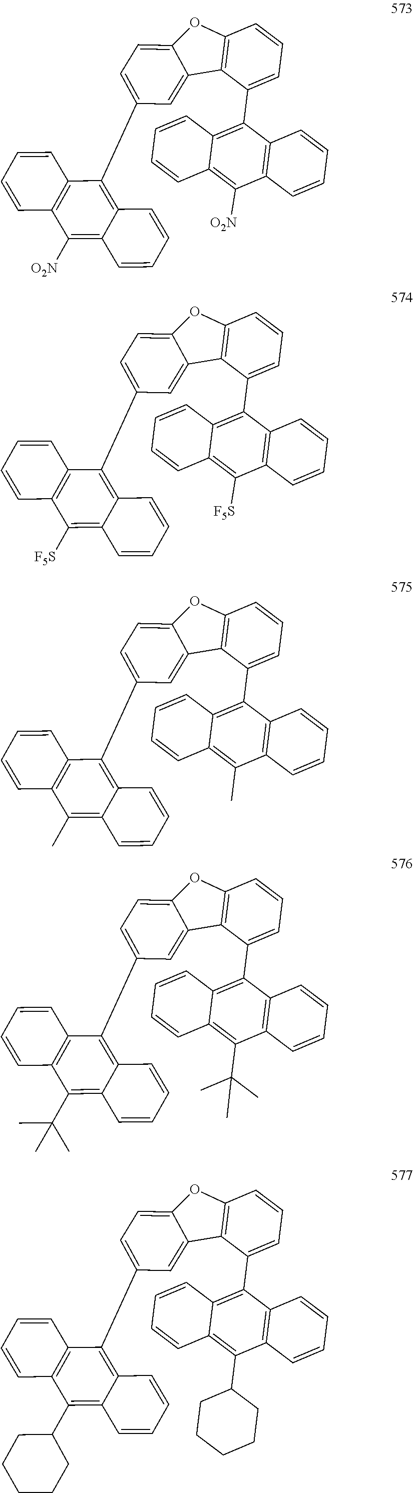

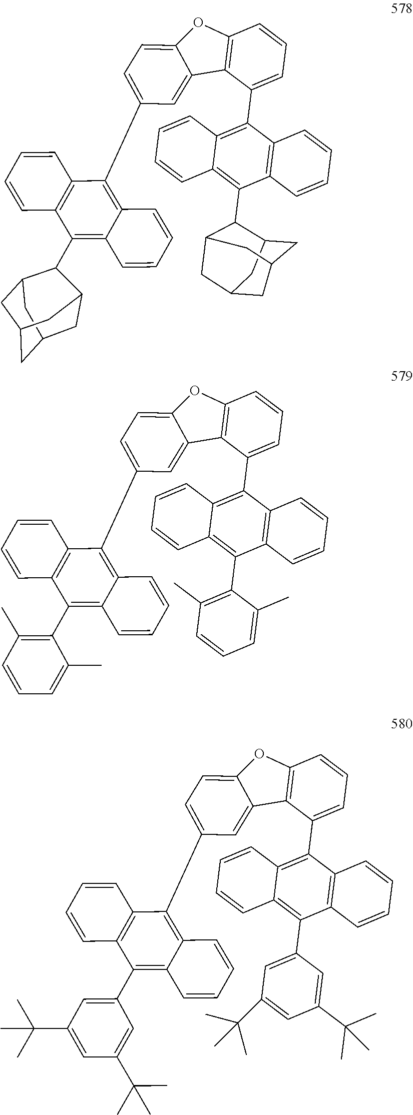 [SMILES: c1cc(-c2c3ccccc3c(C3C4CC5CC(C4)CC3C5)c3ccccc23)c2c(c1)oc1ccc(-c3c4ccccc4c(C4C5CC6CC(C5)CC4C6)c4ccccc34)cc12 ; Cc1cccc(C)c1-c1c2ccccc2c(-c2ccc3oc4cccc(-c5c6ccccc6c(-c6c(C)cccc6C)c6ccccc56)c4c3c2)c2ccccc12 ; CC(C)(C)c1cc(-c2c3ccccc3c(-c3ccc4oc5cccc(-c6c7ccccc7c(-c7cc(C(C)(C)C)cc(C(C)(C)C)c7)c7ccccc67)c5c4c3)c3ccccc23)cc(C(C)(C)C)c1]

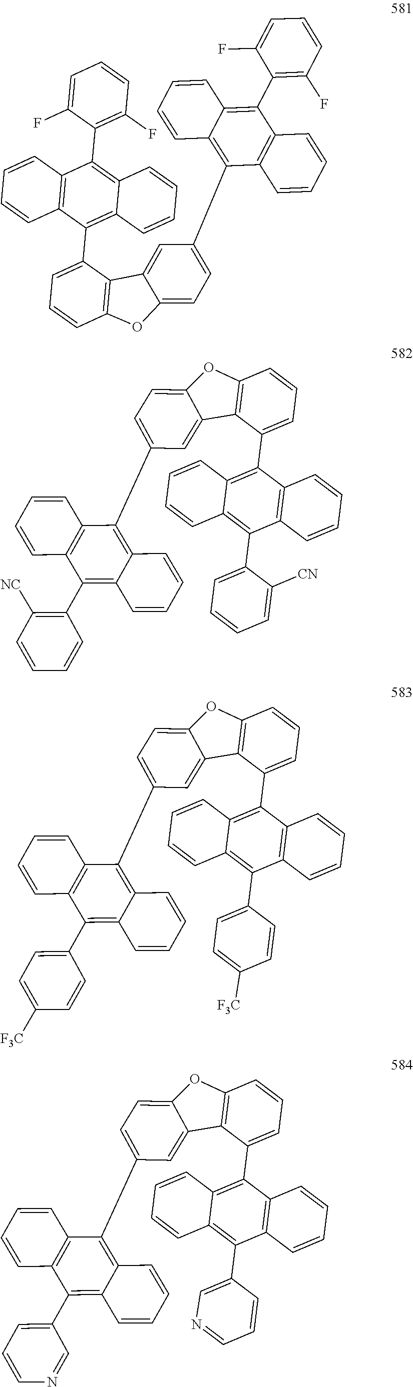 [SMILES: Fc1cccc(F)c1-c1c2ccccc2c(-c2ccc3oc4cccc(-c5c6ccccc6c(-c6c(F)cccc6F)c6ccccc56)c4c3c2)c2ccccc12 ; N#Cc1ccccc1-c1c2ccccc2c(-c2ccc3oc4cccc(-c5c6ccccc6c(-c6ccccc6C#N)c6ccccc56)c4c3c2)c2ccccc12 ; FC(F)(F)c1ccc(-c2c3ccccc3c(-c3ccc4oc5cccc(-c6c7ccccc7c(-c7ccc(C(F)(F)F)cc7)c7ccccc67)c5c4c3)c3ccccc23)cc1 ; c1cncc(-c2c3ccccc3c(-c3ccc4oc5cccc(-c6c7ccccc7c(-c7cccnc7)c7ccccc67)c5c4c3)c3ccccc23)c1]

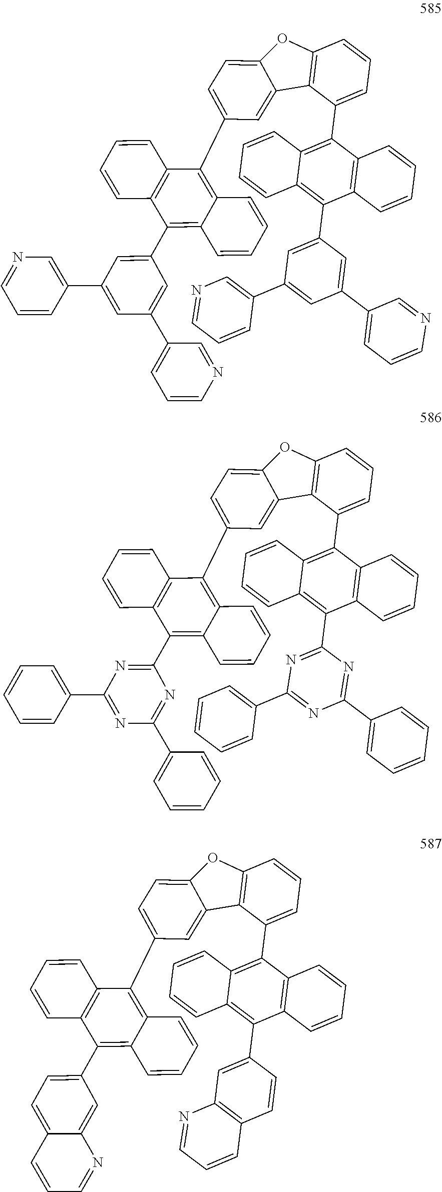 [SMILES: c1cncc(-c2cc(-c3cccnc3)cc(-c3c4ccccc4c(-c4ccc5oc6cccc(-c7c8ccccc8c(-c8cc(-c9cccnc9)cc(-c9cccnc9)c8)c8ccccc78)c6c5c4)c4ccccc34)c2)c1 ; c1ccc(-c2nc(-c3ccccc3)nc(-c3c4ccccc4c(-c4ccc5oc6cccc(-c7c8ccccc8c(-c8nc(-c9ccccc9)nc(-c9ccccc9)n8)c8ccccc78)c6c5c4)c4ccccc34)n2)cc1 ; c1cnc2cc(-c3c4ccccc4c(-c4ccc5oc6cccc(-c7c8ccccc8c(-c8ccc9cccnc9c8)c8ccccc78)c6c5c4)c4ccccc34)ccc2c1]

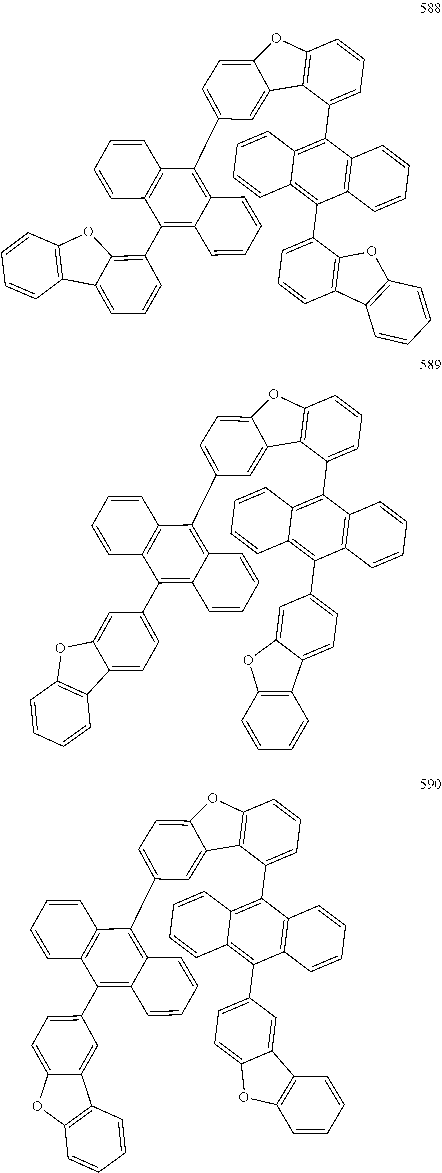 [SMILES: c1ccc2c(c1)oc1c(-c3c4ccccc4c(-c4ccc5oc6cccc(-c7c8ccccc8c(-c8cccc9c8oc8ccccc89)c8ccccc78)c6c5c4)c4ccccc34)cccc12 ; c1ccc2c(c1)oc1cc(-c3c4ccccc4c(-c4ccc5oc6cccc(-c7c8ccccc8c(-c8ccc9c(c8)oc8ccccc89)c8ccccc78)c6c5c4)c4ccccc34)ccc12 ; c1ccc2c(c1)oc1ccc(-c3c4ccccc4c(-c4ccc5oc6cccc(-c7c8ccccc8c(-c8ccc9oc%10ccccc%10c9c8)c8ccccc78)c6c5c4)c4ccccc34)cc12]

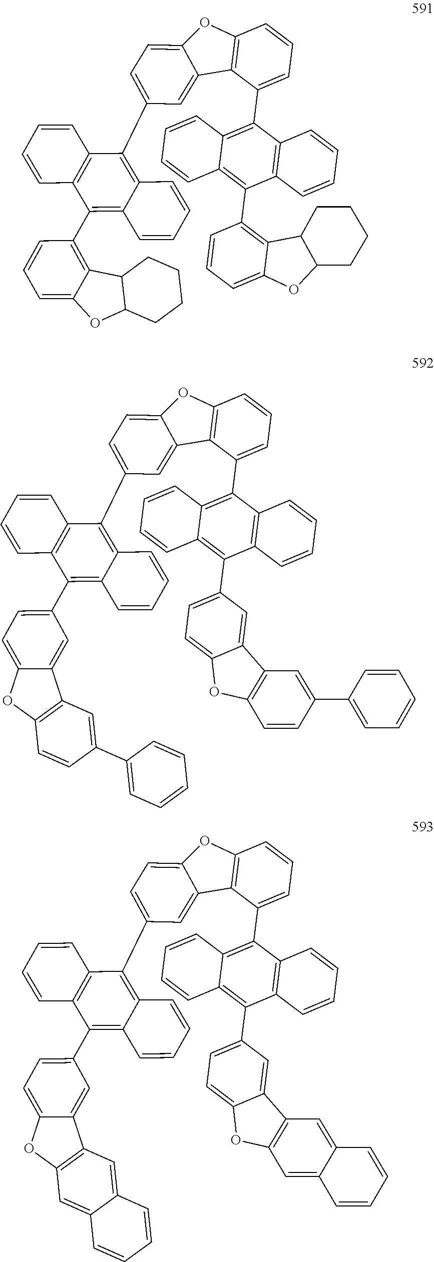 [SMILES: c1cc2c(c(-c3c4ccccc4c(-c4ccc5oc6cccc(-c7c8ccccc8c(-c8cccc9c8C8CCCCC8O9)c8ccccc78)c6c5c4)c4ccccc34)c1)C1CCCCC1O2 ; c1ccc(-c2ccc3oc4ccc(-c5c6ccccc6c(-c6ccc7oc8cccc(-c9c%10ccccc%10c(-c%10ccc%11oc%12ccc(-c%13ccccc%13)cc%12c%11c%10)c%10ccccc9%10)c8c7c6)c6ccccc56)cc4c3c2)cc1 ; c1ccc2cc3c(cc2c1)oc1ccc(-c2c4ccccc4c(-c4ccc5oc6cccc(-c7c8ccccc8c(-c8ccc9oc%10cc%11ccccc%11cc%10c9c8)c8ccccc78)c6c5c4)c4ccccc24)cc13]

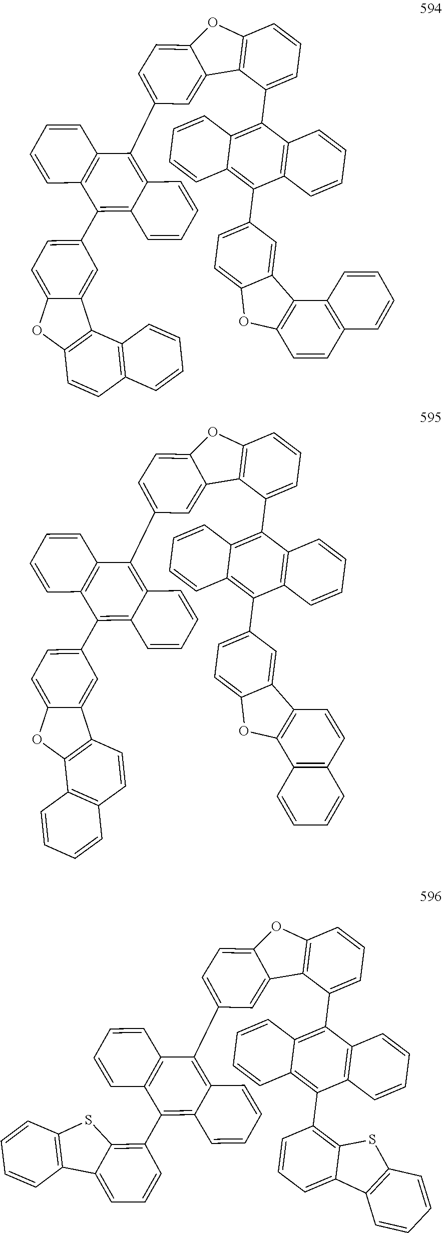 [SMILES: c1ccc2c(c1)ccc1oc3ccc(-c4c5ccccc5c(-c5ccc6oc7cccc(-c8c9ccccc9c(-c9ccc%10oc%11ccc%12ccccc%12c%11c%10c9)c9ccccc89)c7c6c5)c5ccccc45)cc3c12 ; c1ccc2c(c1)ccc1c3cc(-c4c5ccccc5c(-c5ccc6oc7cccc(-c8c9ccccc9c(-c9ccc%10oc%11c%12ccccc%12ccc%11c%10c9)c9ccccc89)c7c6c5)c5ccccc45)ccc3oc21 ; c1cc(-c2c3ccccc3c(-c3cccc4c3sc3ccccc34)c3ccccc23)c2c(c1)oc1ccc(-c3c4ccccc4c(-c4cccc5c4sc4ccccc45)c4ccccc34)cc12]

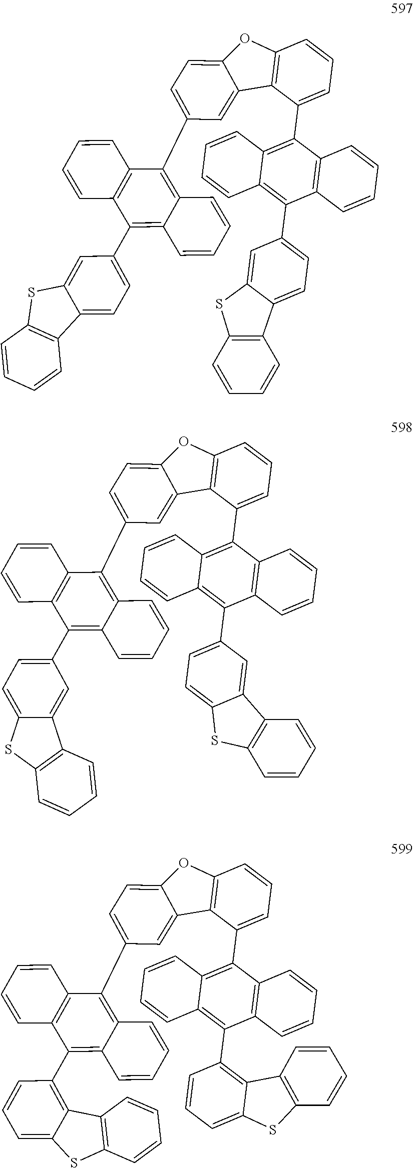 [SMILES: c1cc(-c2c3ccccc3c(-c3ccc4c(c3)sc3ccccc34)c3ccccc23)c2c(c1)oc1ccc(-c3c4ccccc4c(-c4ccc5c(c4)sc4ccccc45)c4ccccc34)cc12 ; c1cc(-c2c3ccccc3c(-c3ccc4sc5ccccc5c4c3)c3ccccc23)c2c(c1)oc1ccc(-c3c4ccccc4c(-c4ccc5sc6ccccc6c5c4)c4ccccc34)cc12 ; c1cc(-c2c3ccccc3c(-c3cccc4sc5ccccc5c34)c3ccccc23)c2c(c1)oc1ccc(-c3c4ccccc4c(-c4cccc5sc6ccccc6c45)c4ccccc34)cc12]

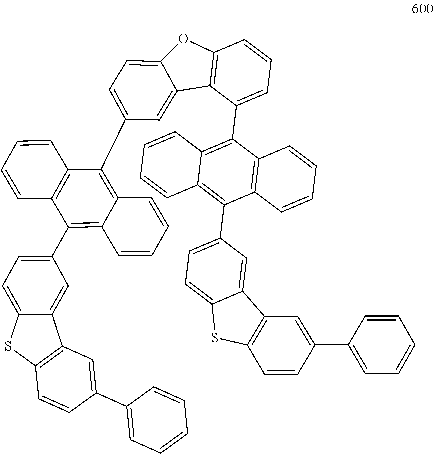 [SMILES: c1ccc(-c2ccc3sc4ccc(-c5c6ccccc6c(-c6ccc7oc8cccc(-c9c%10ccccc%10c(-c%10ccc%11sc%12ccc(-c%13ccccc%13)cc%12c%11c%10)c%10ccccc9%10)c8c7c6)c6ccccc56)cc4c3c2)cc1]

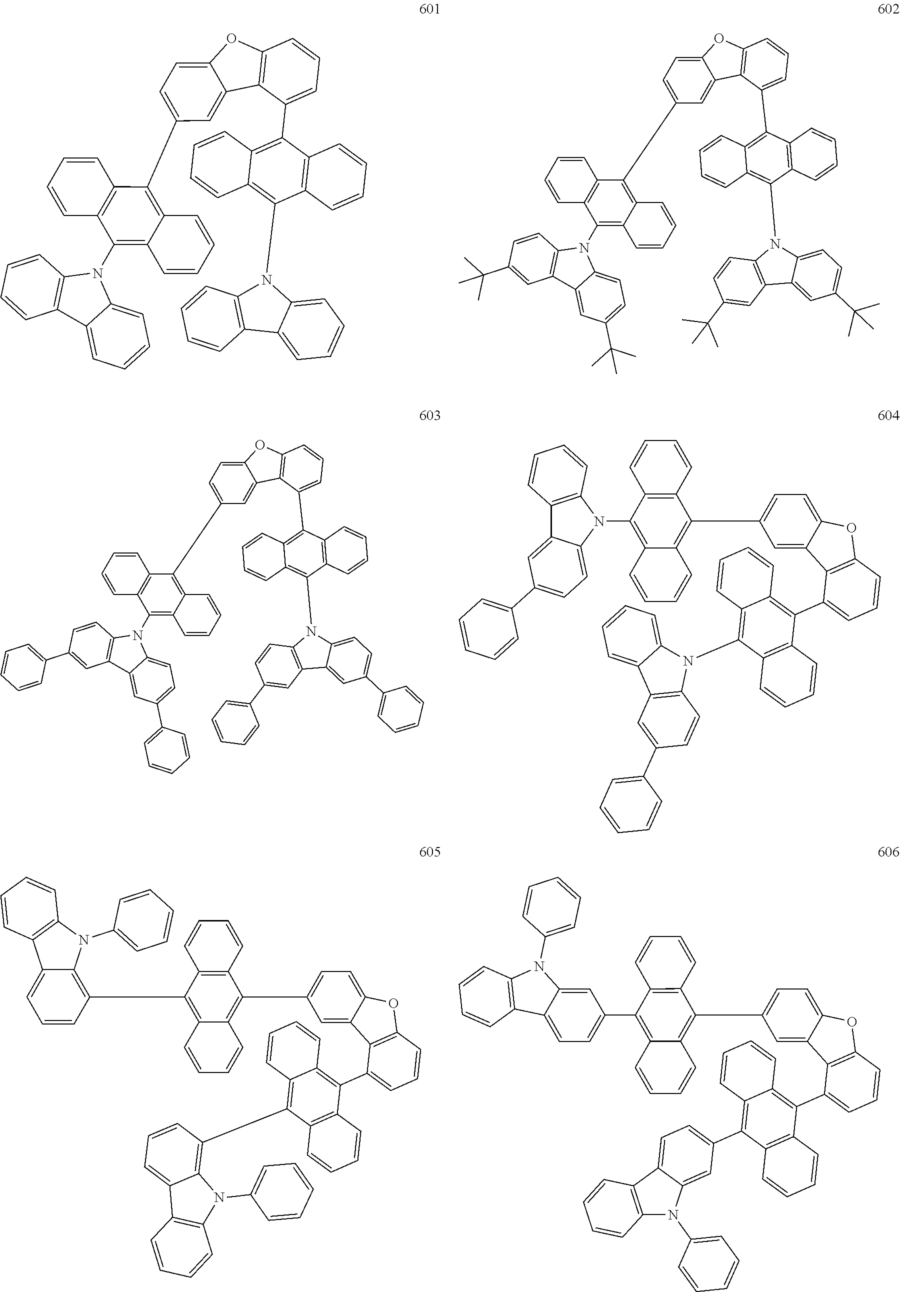

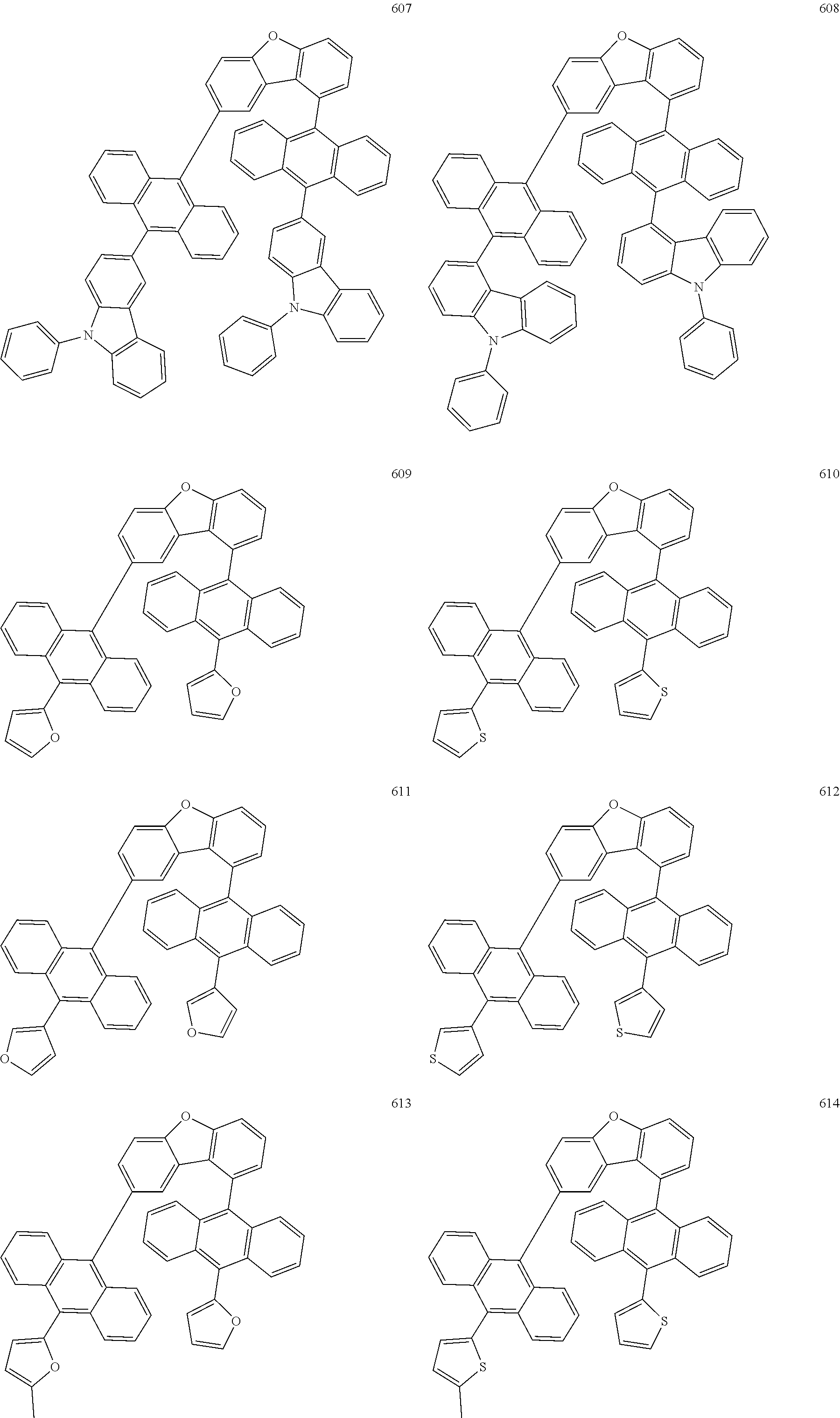

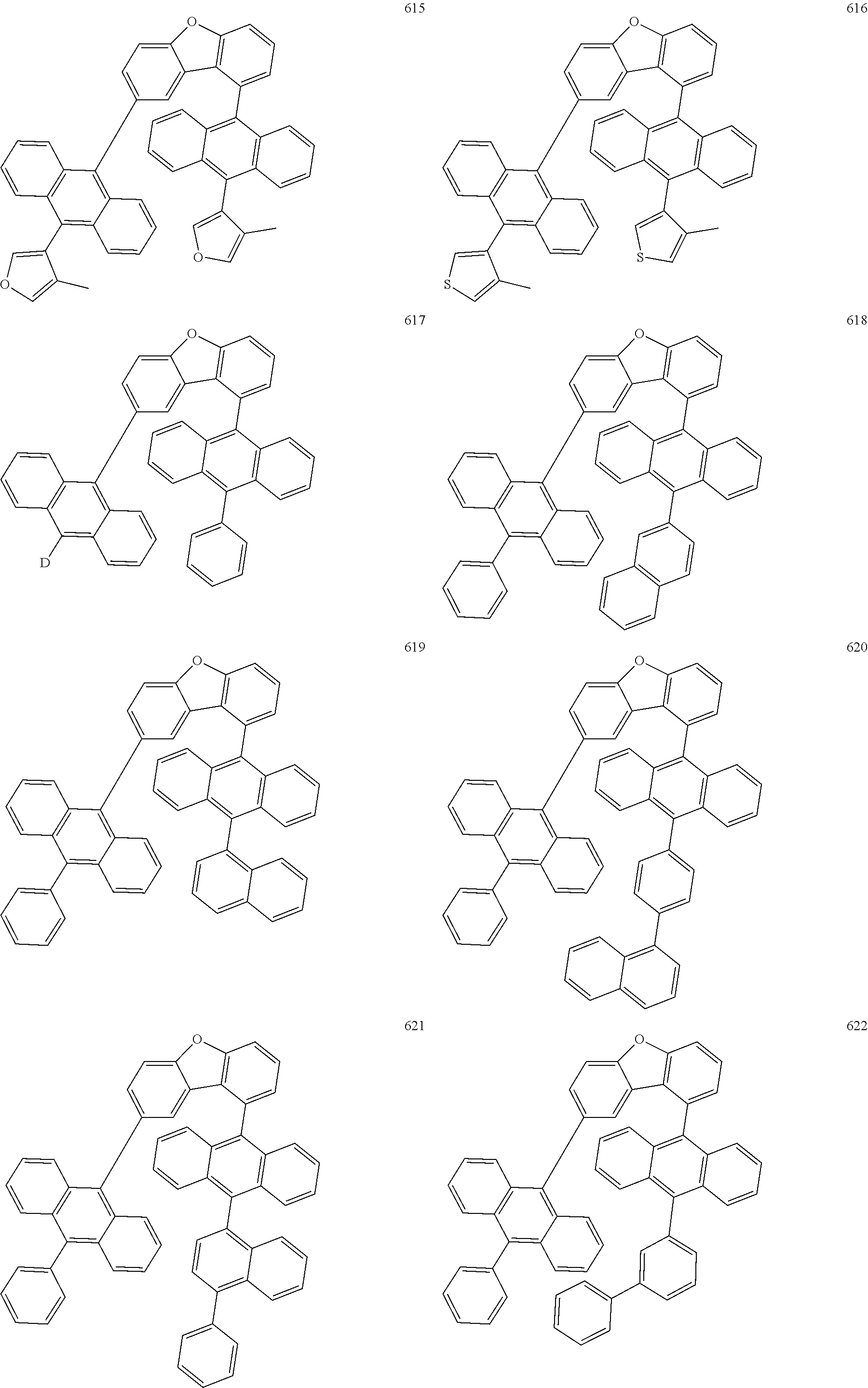

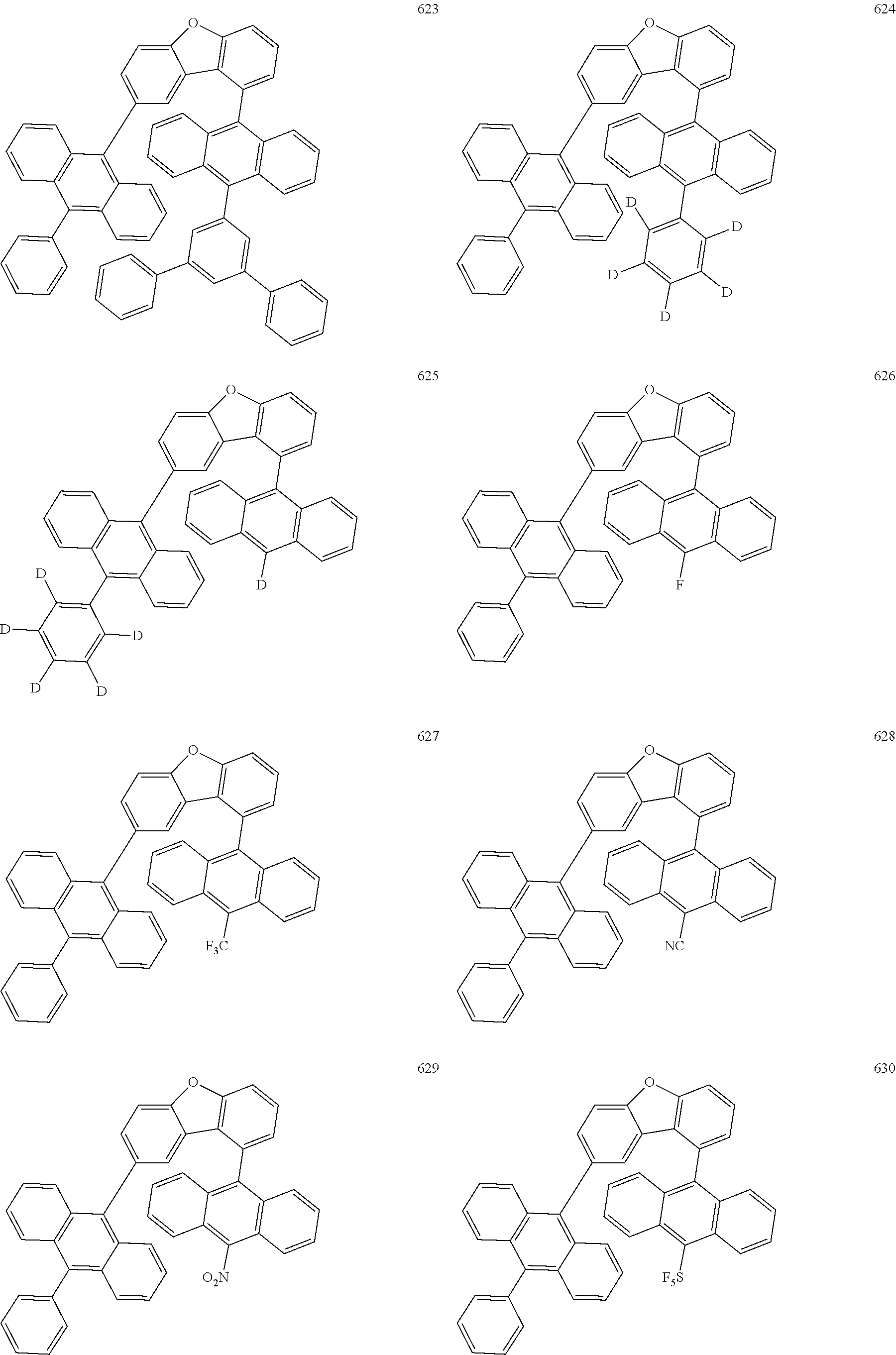

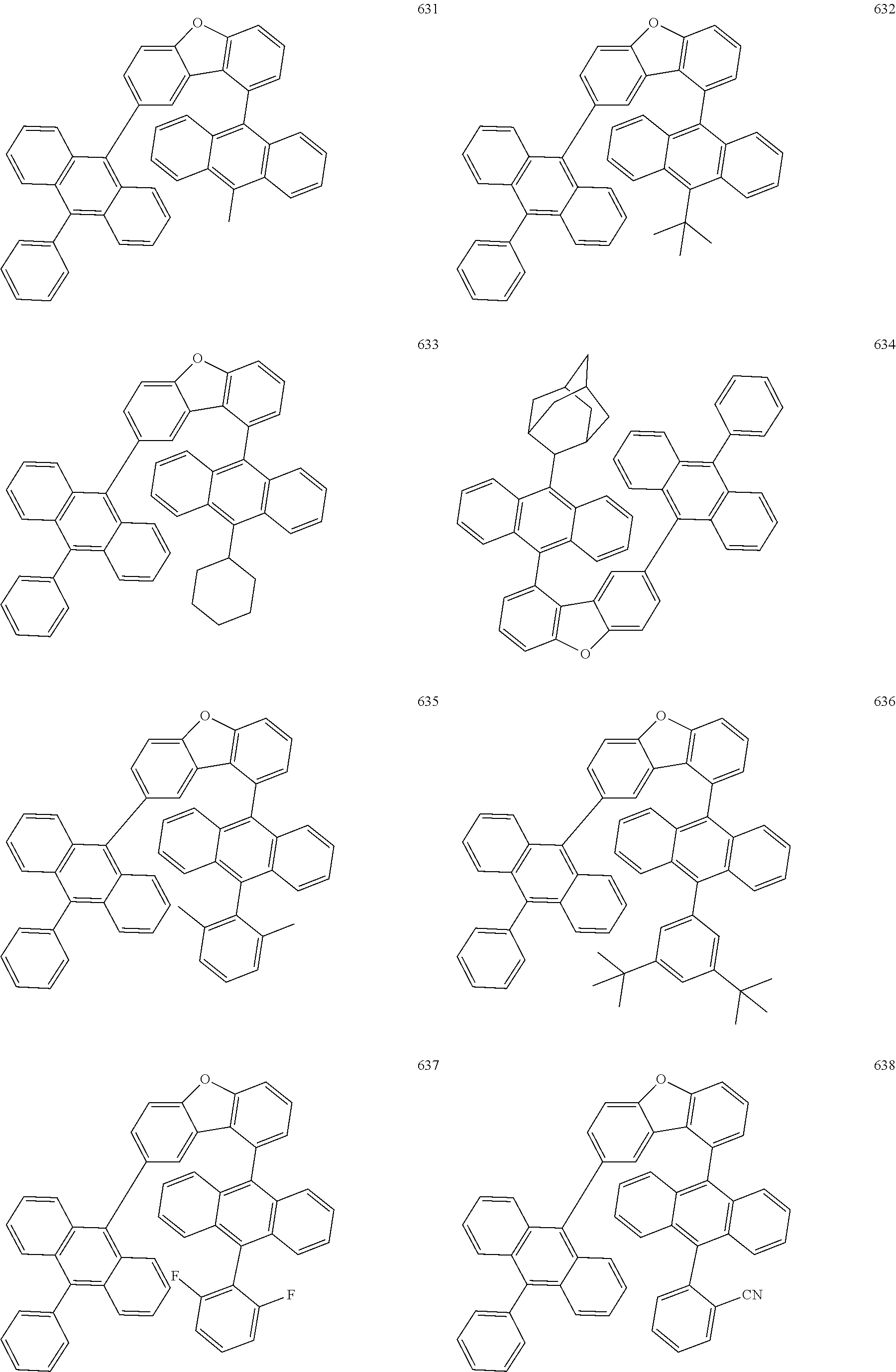

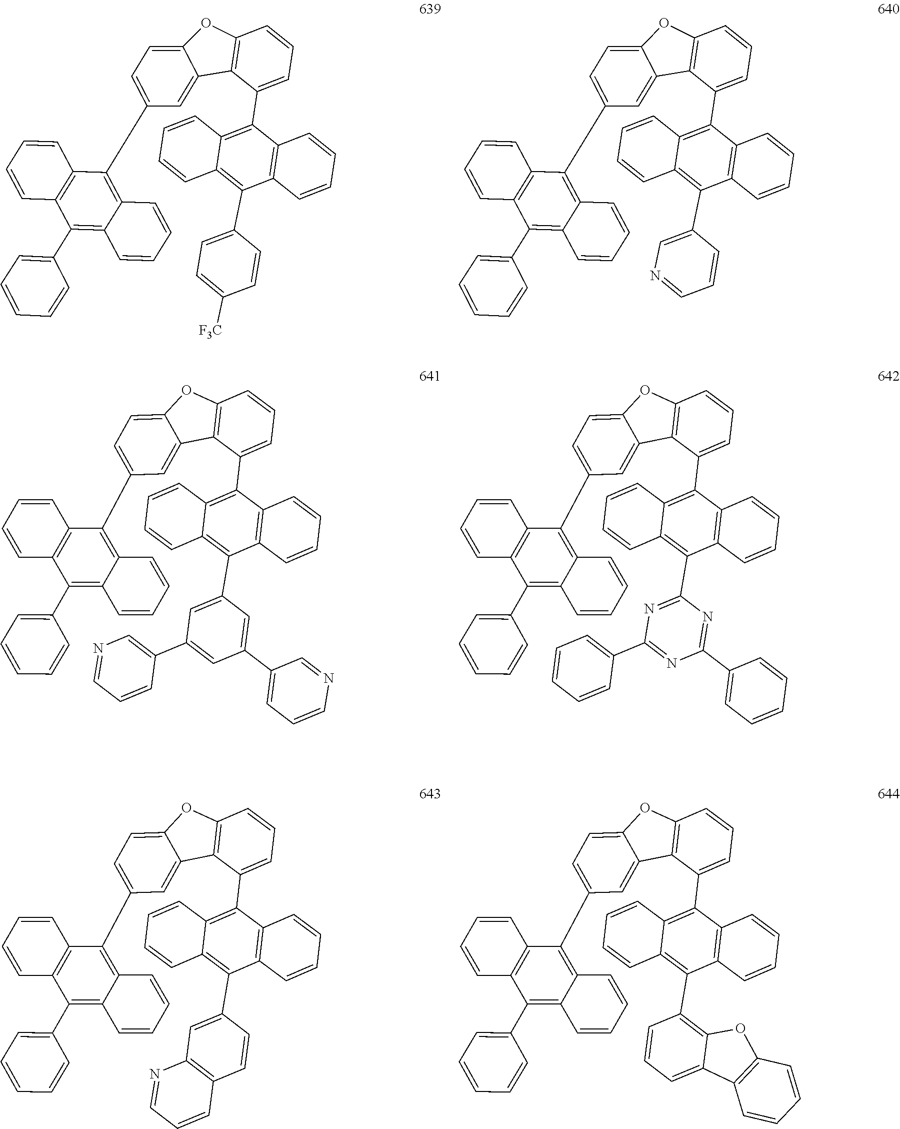

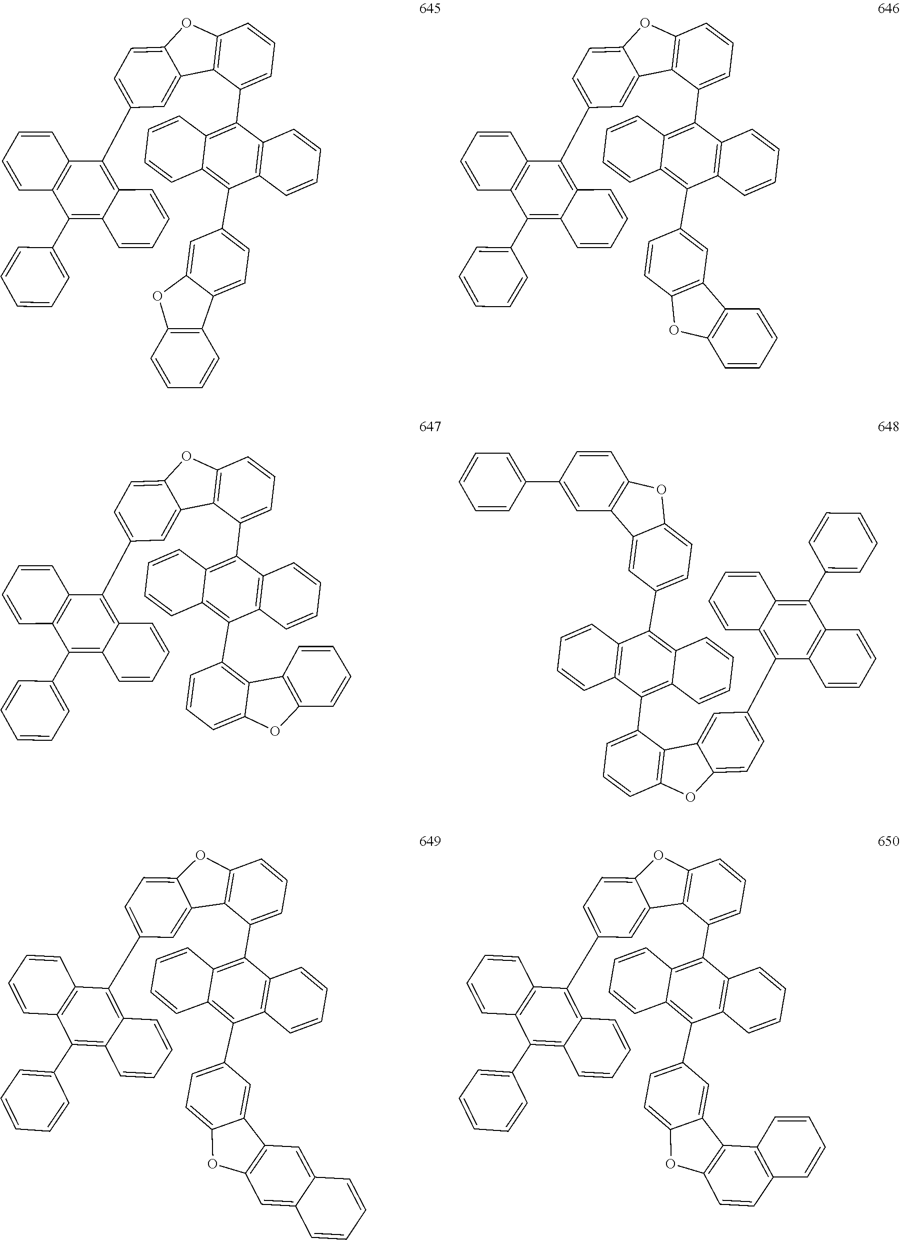

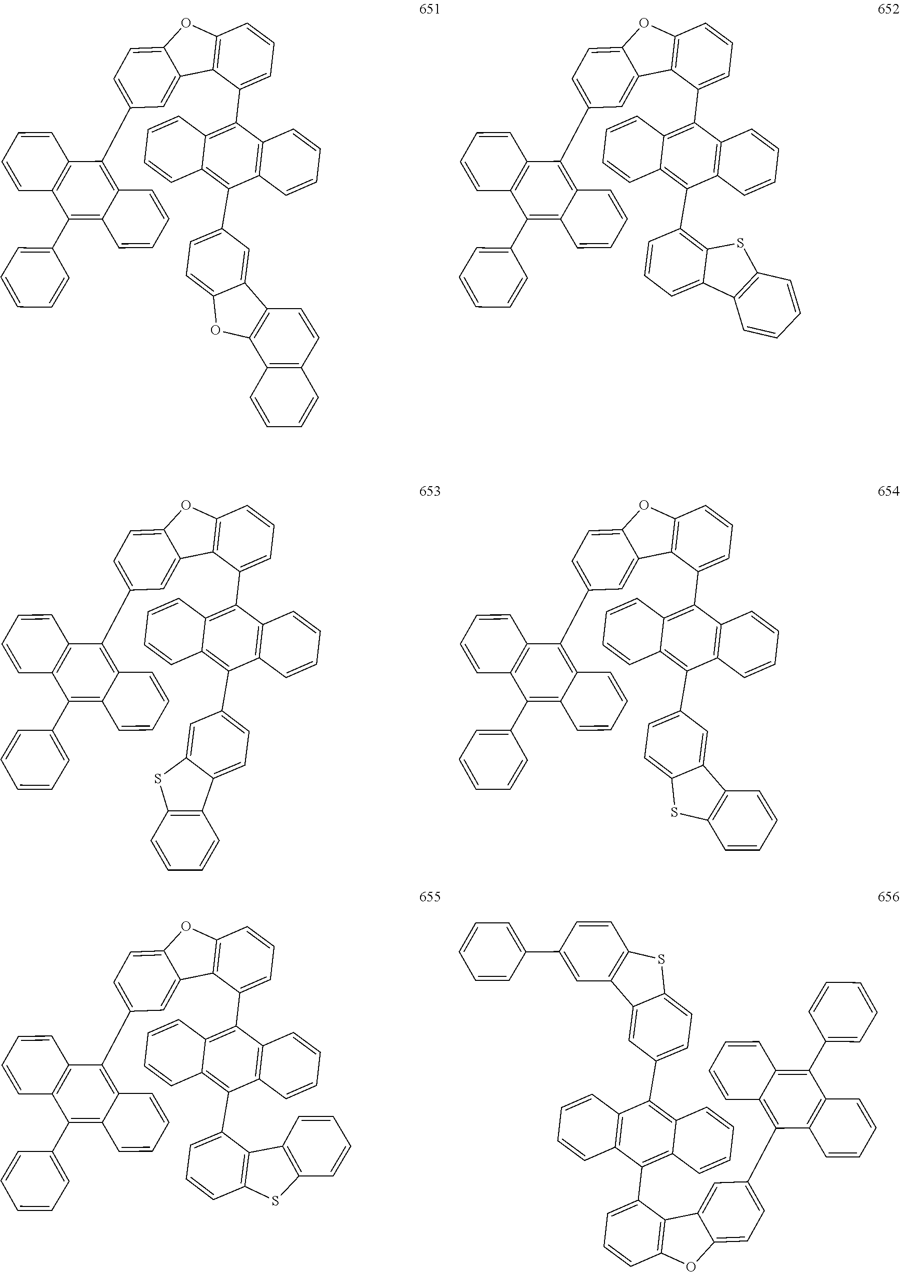

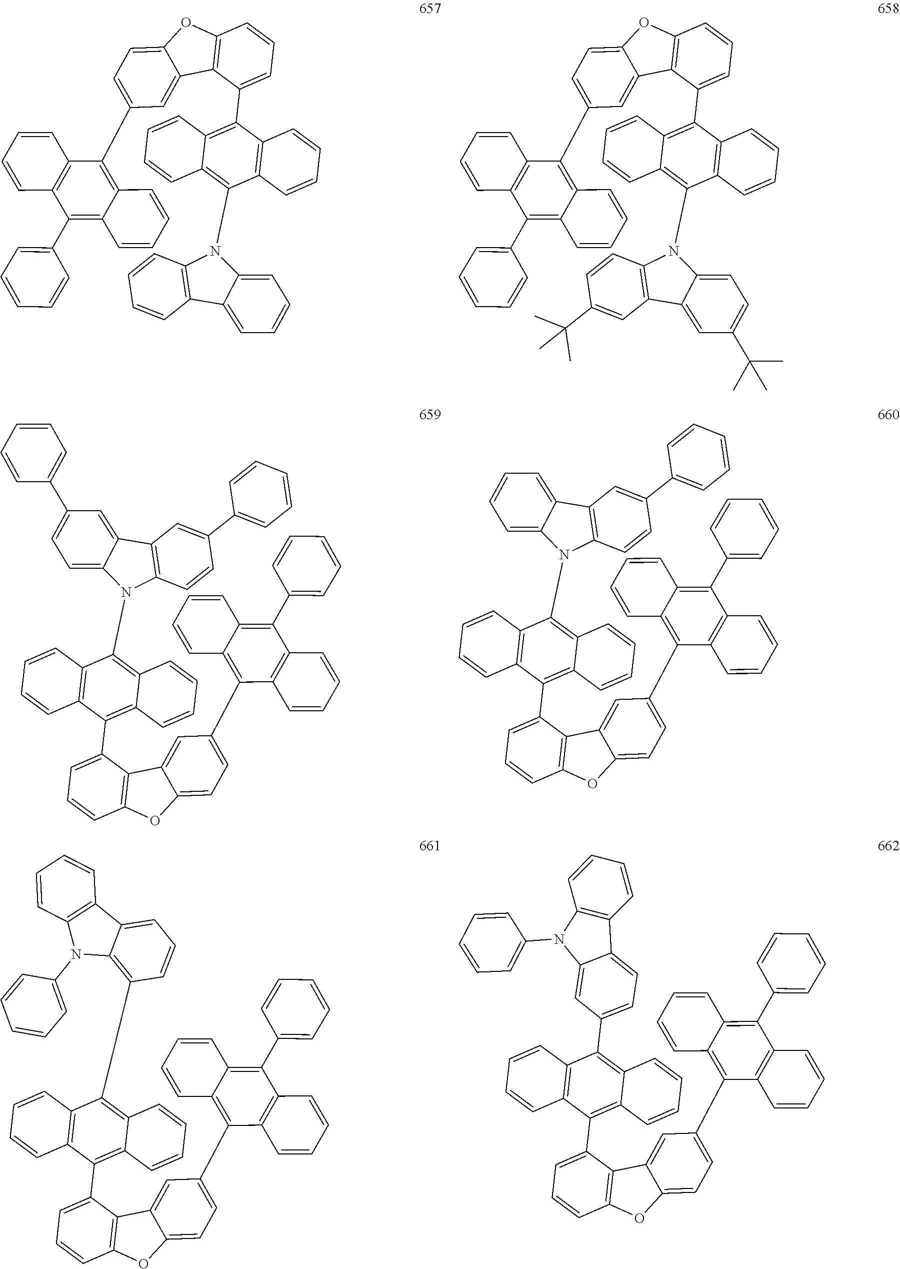

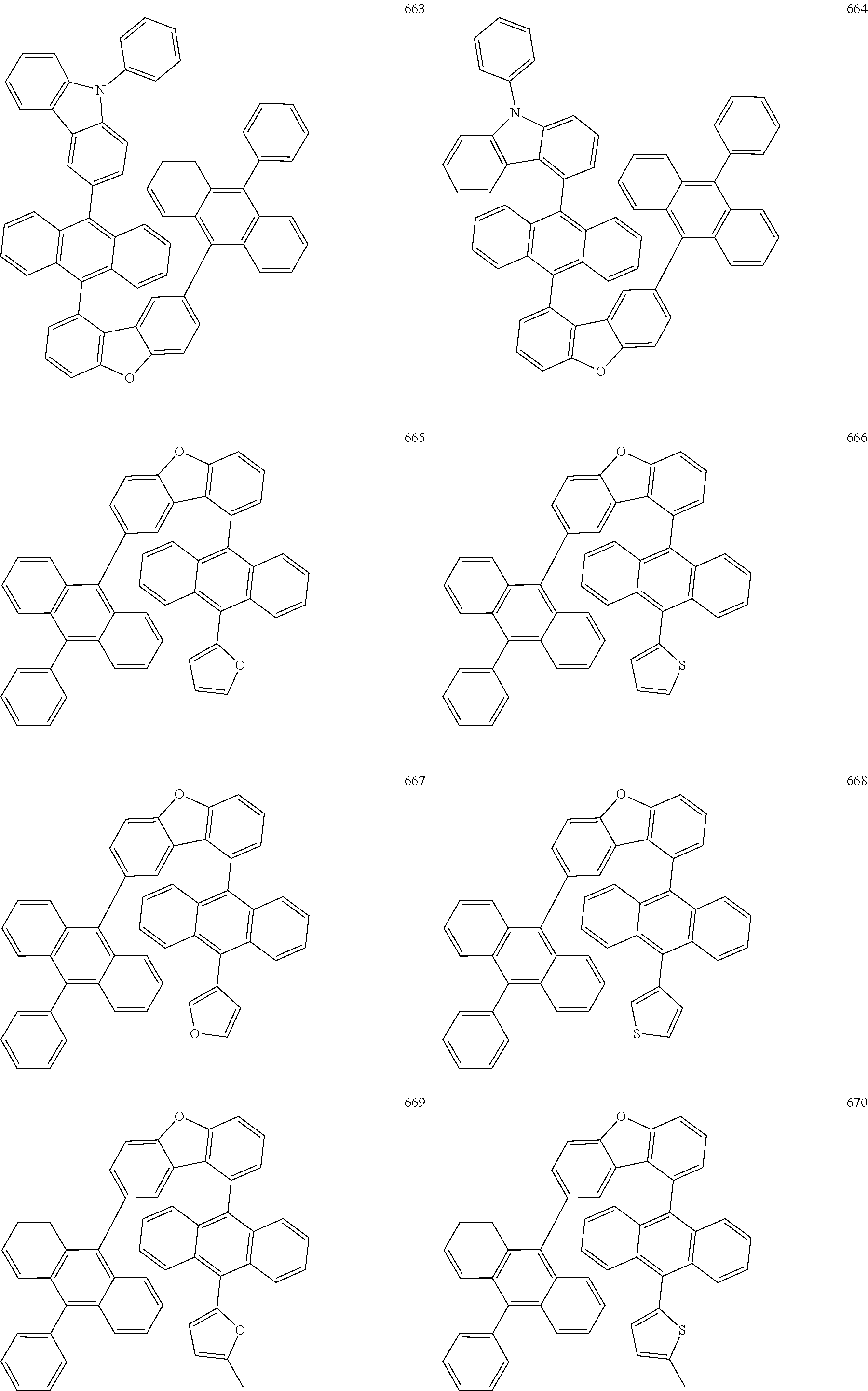

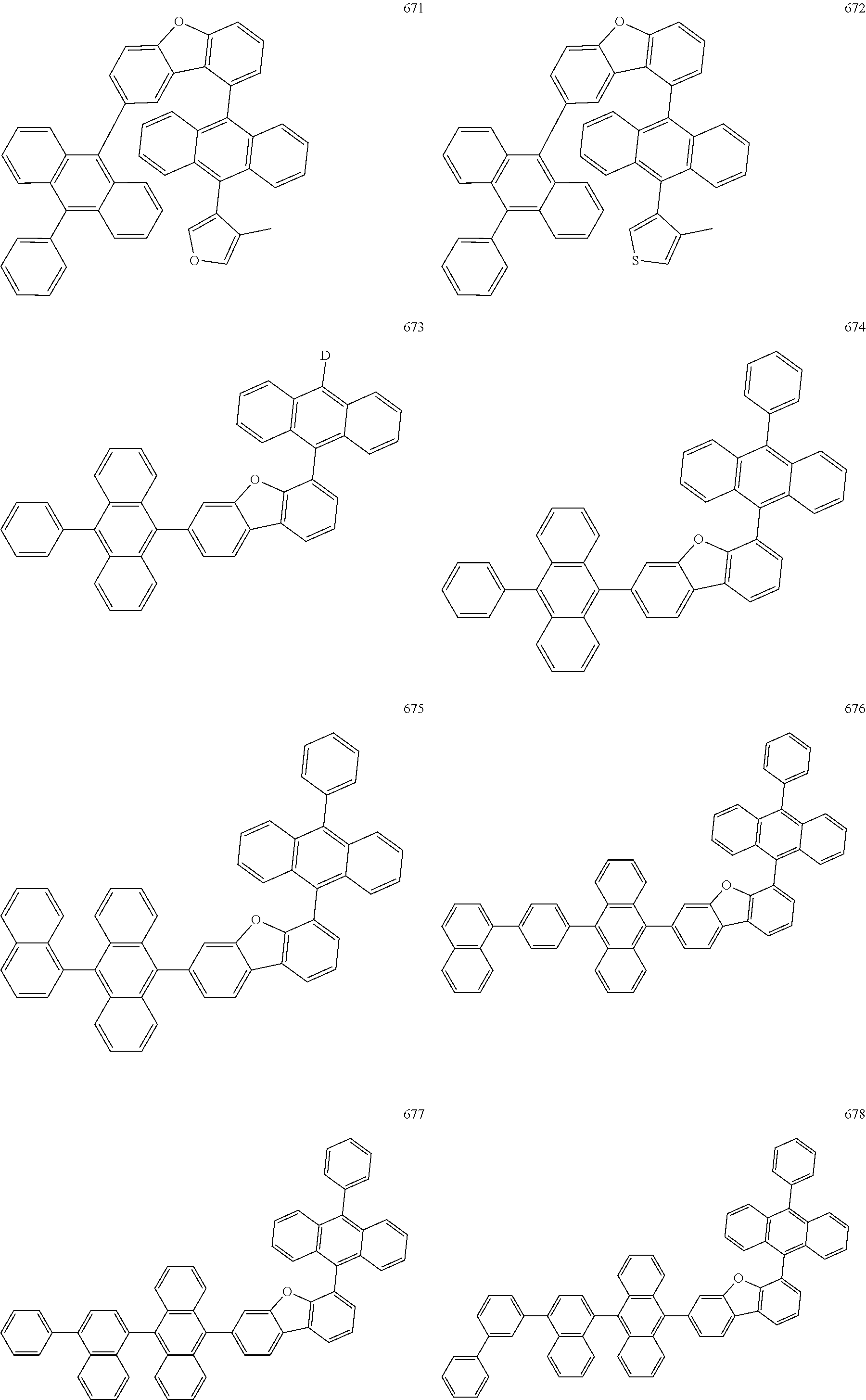

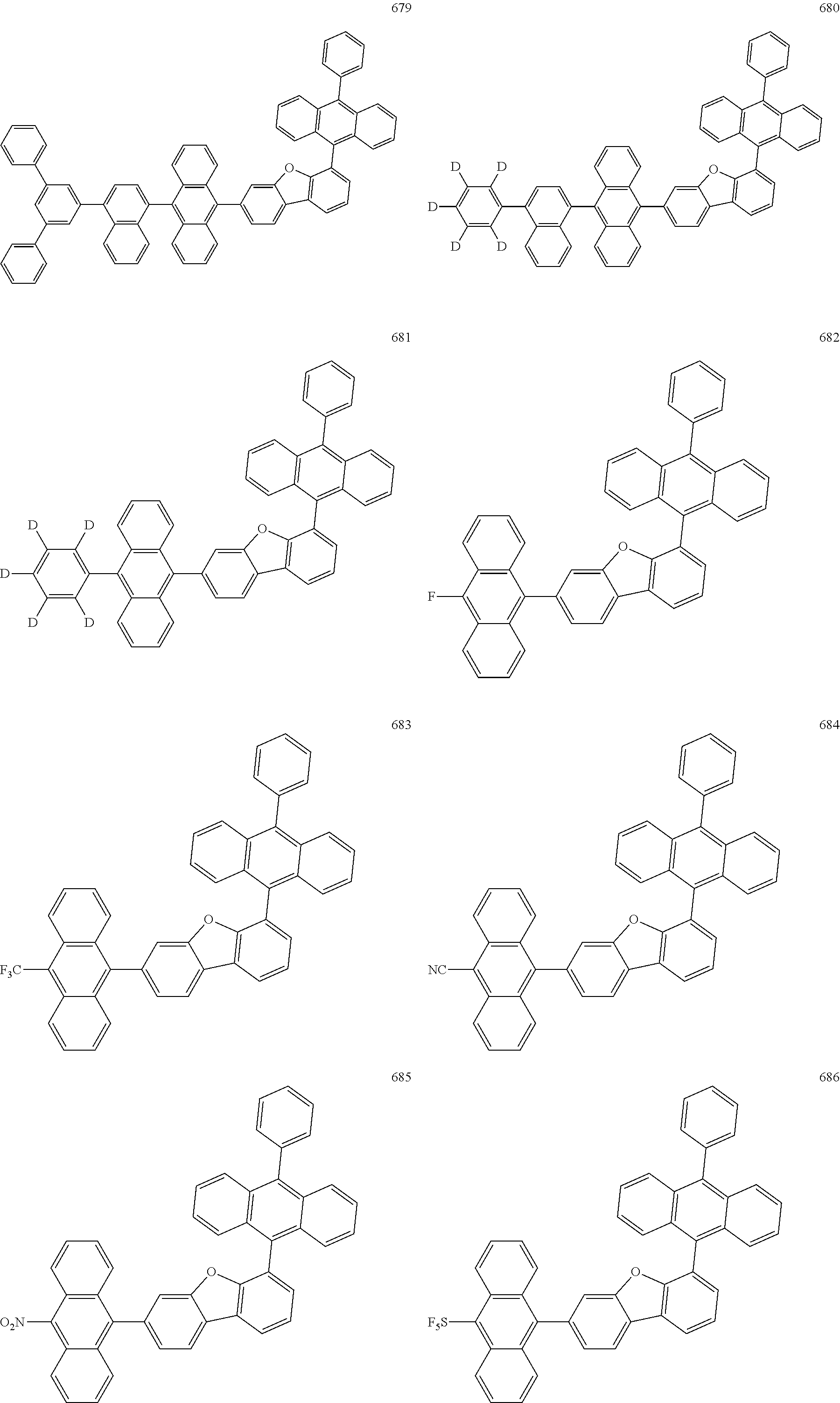

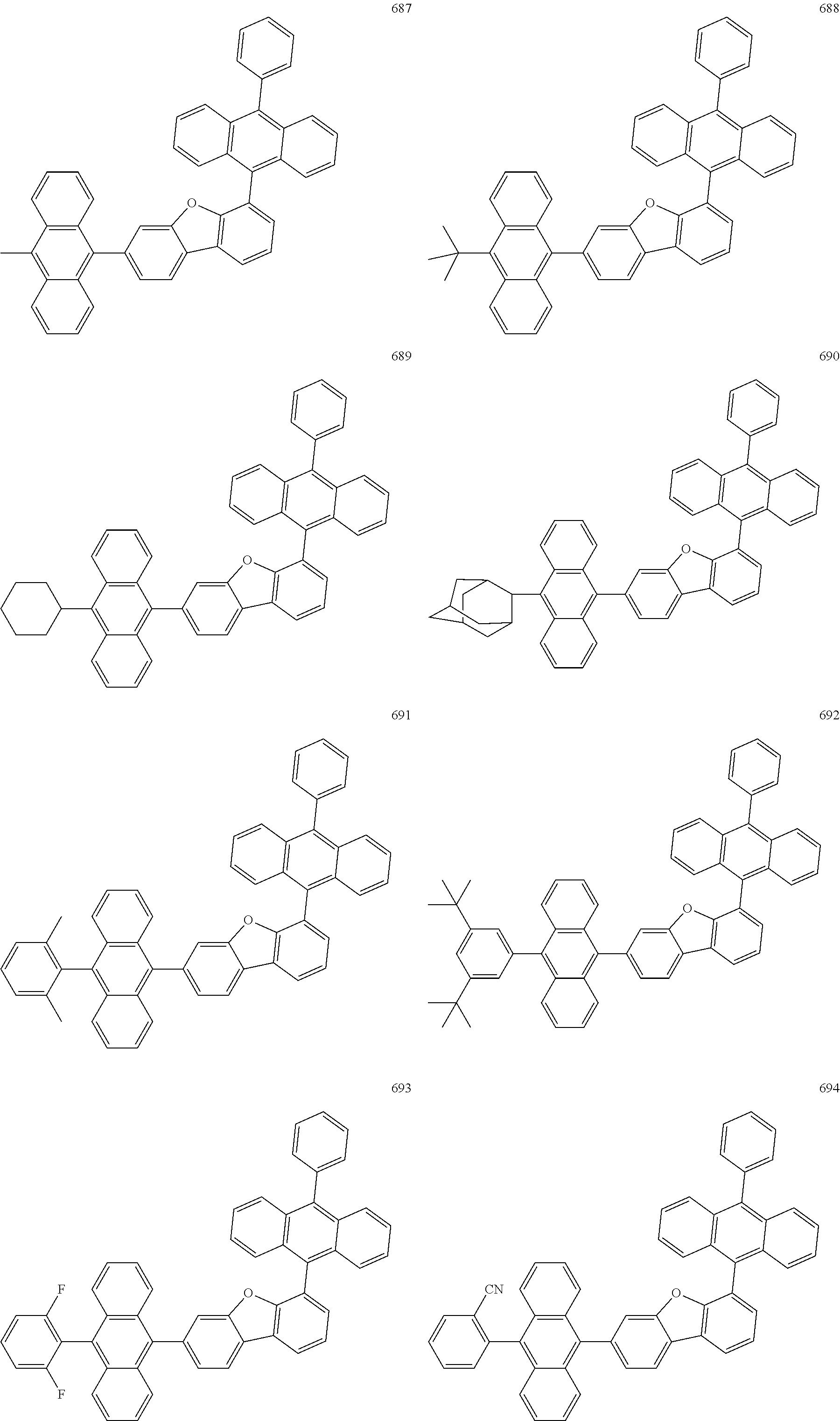

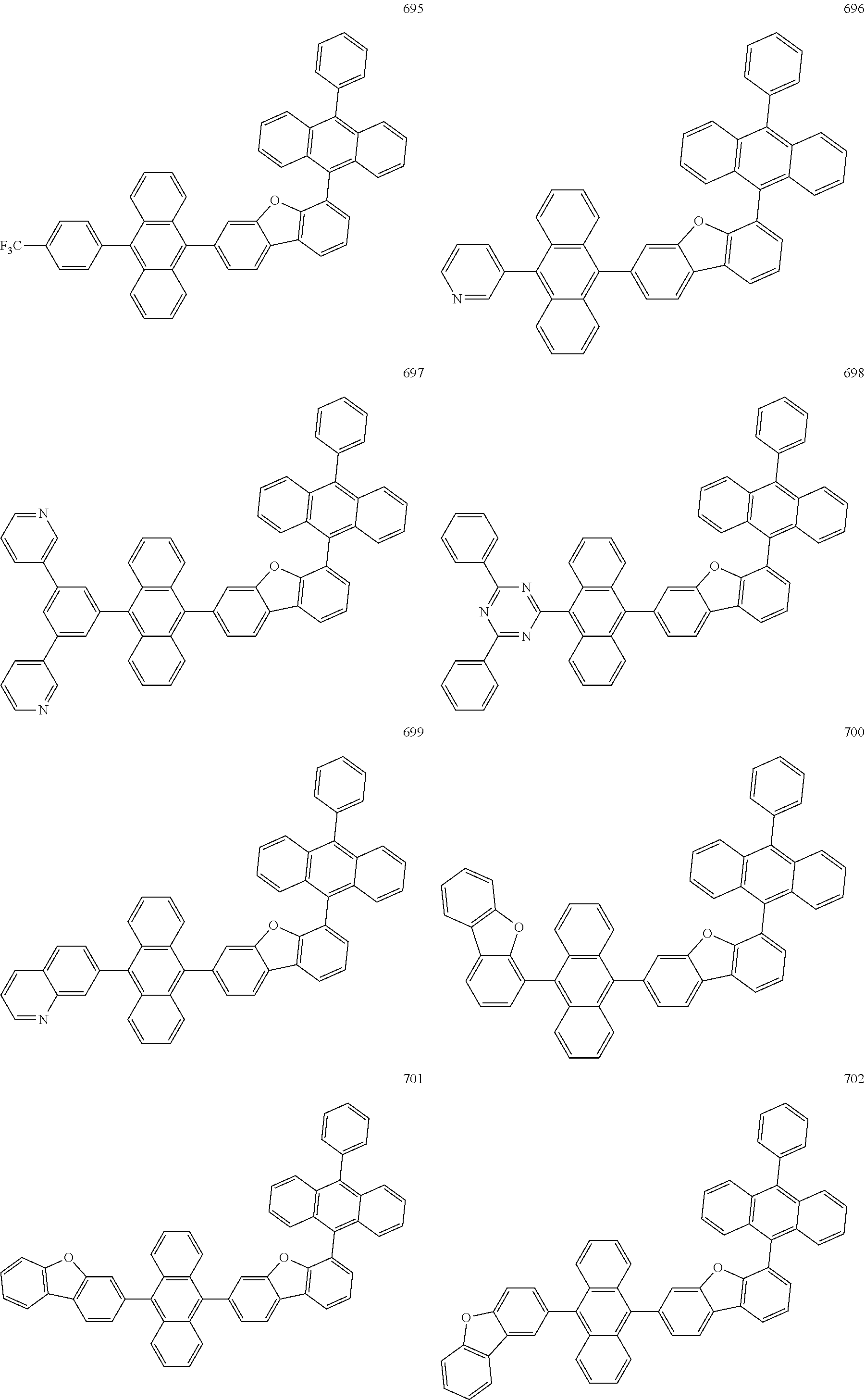

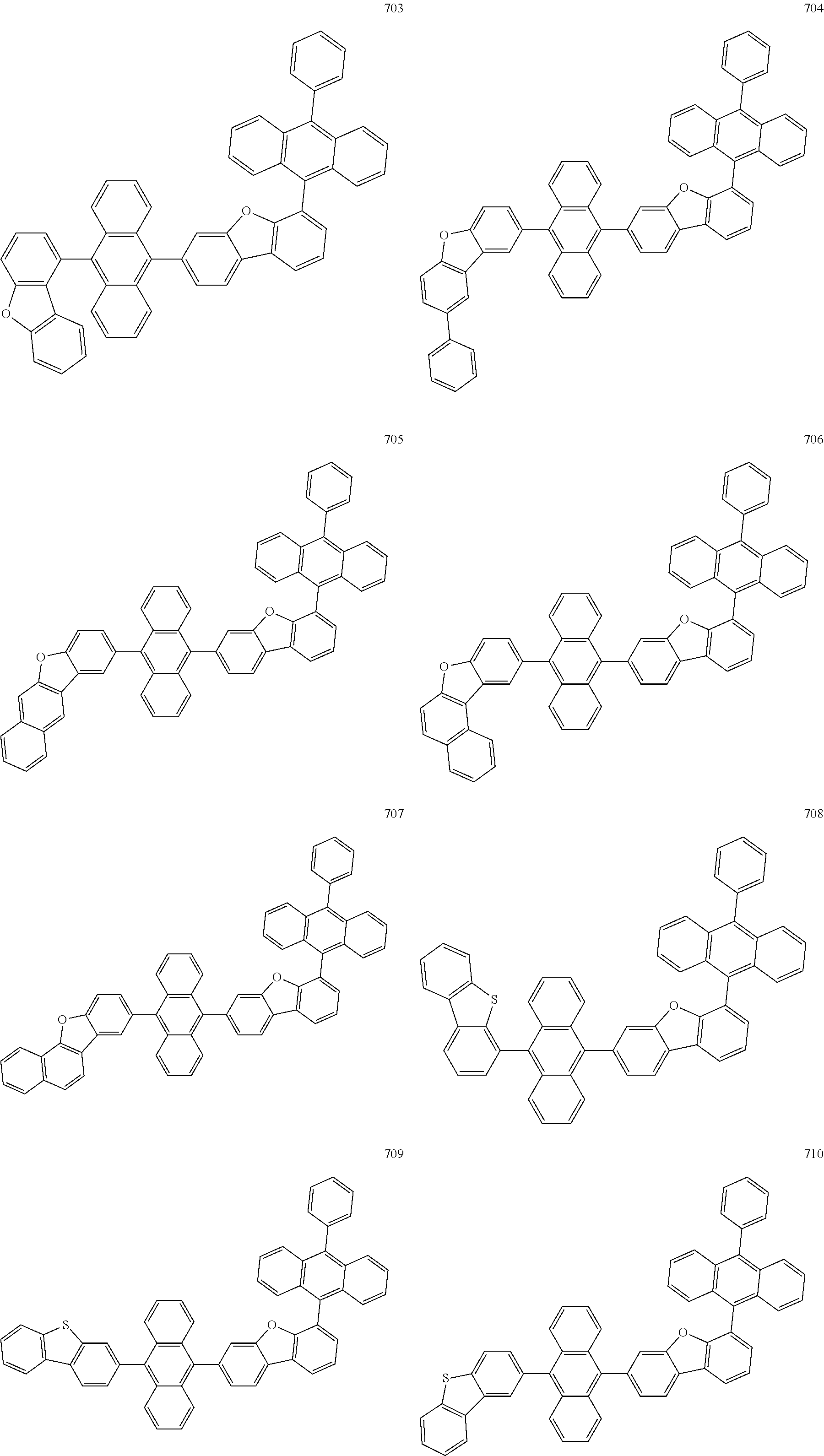

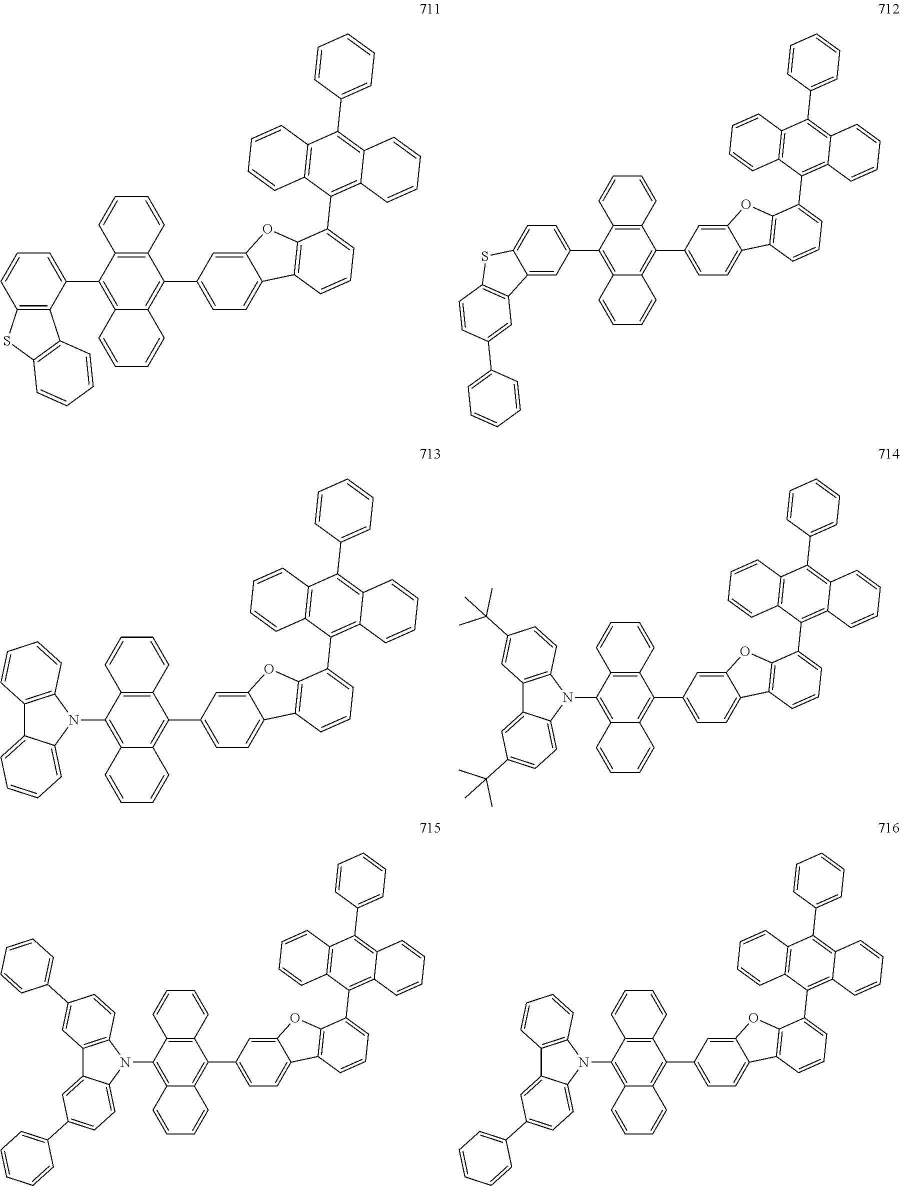

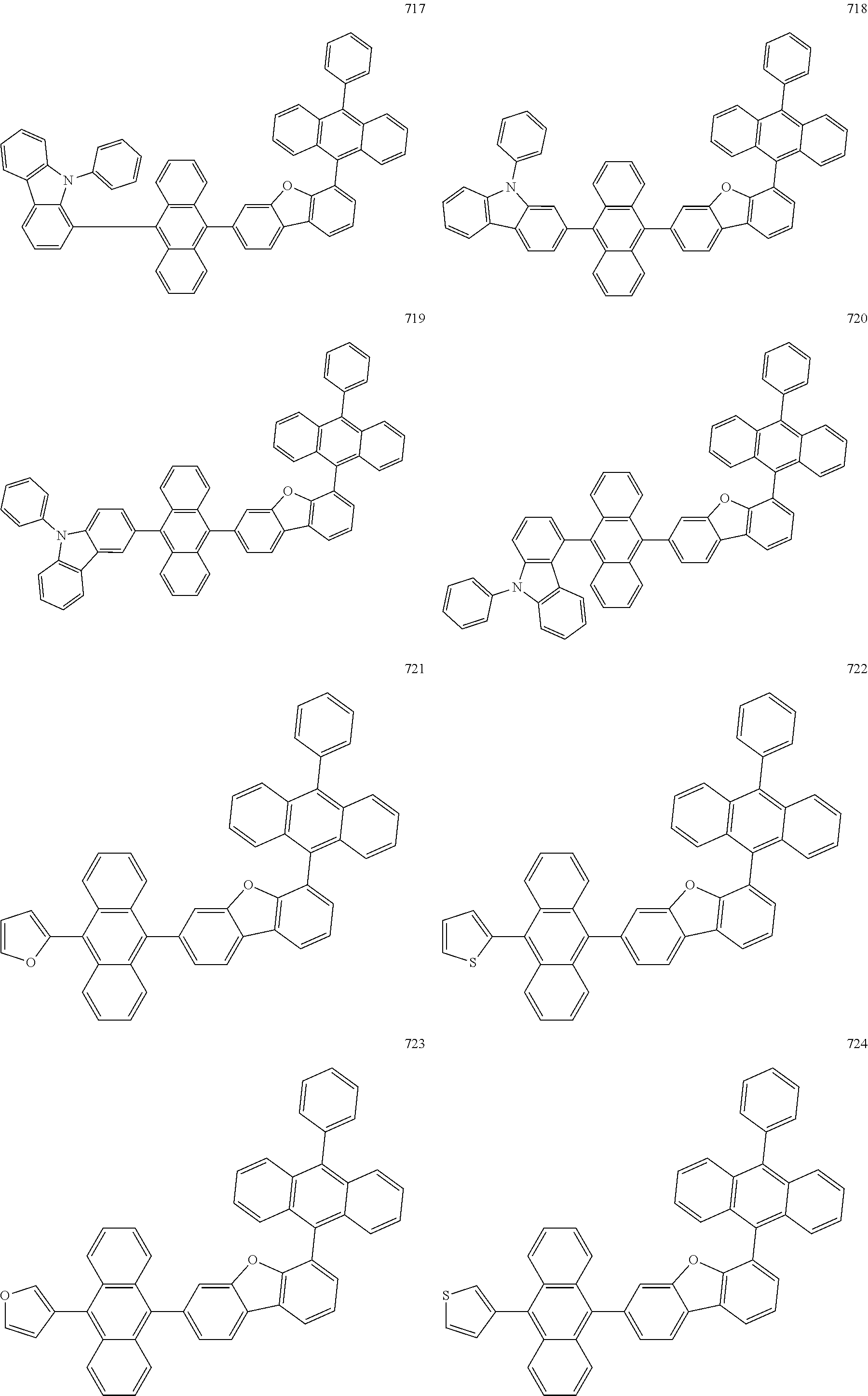

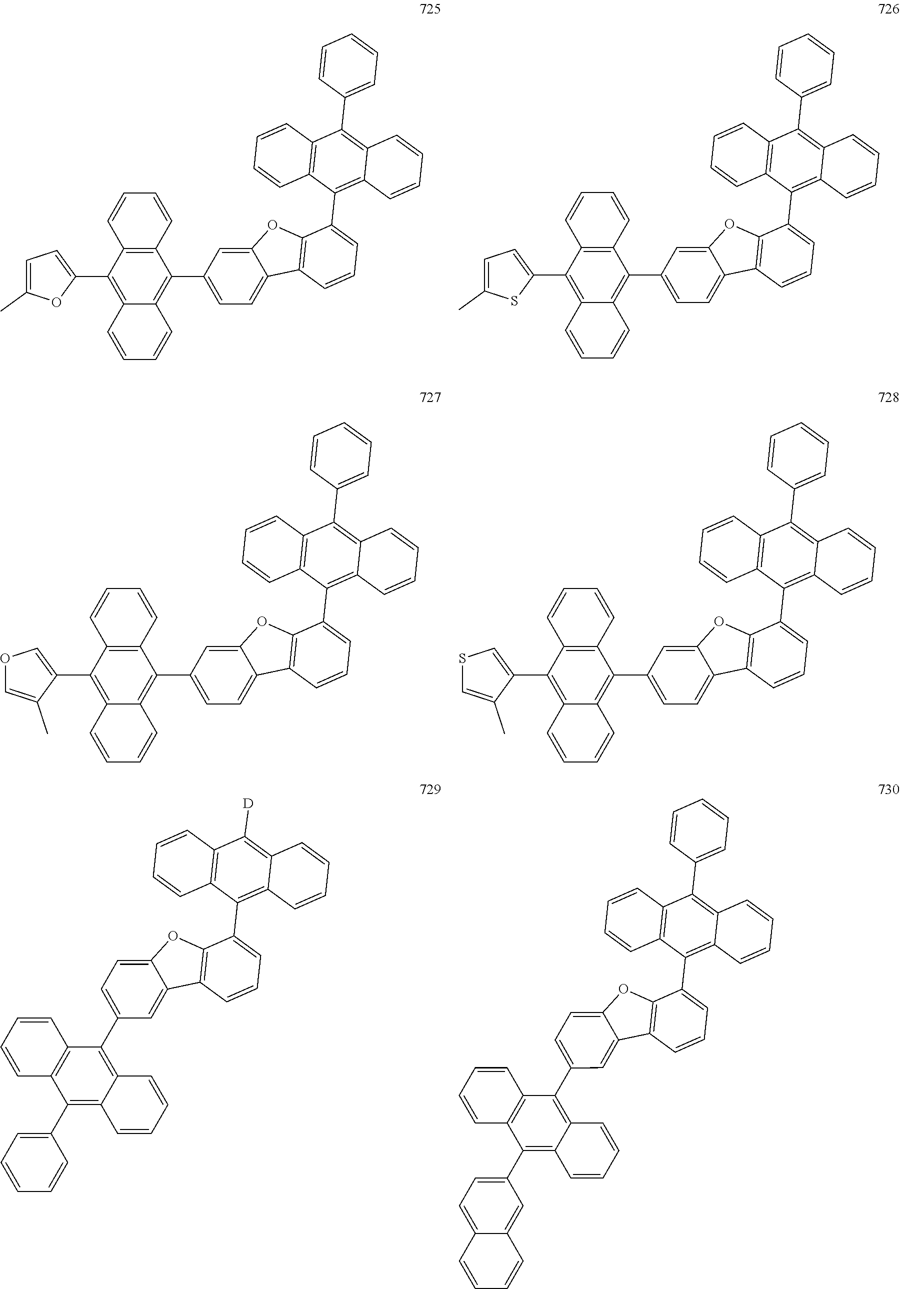

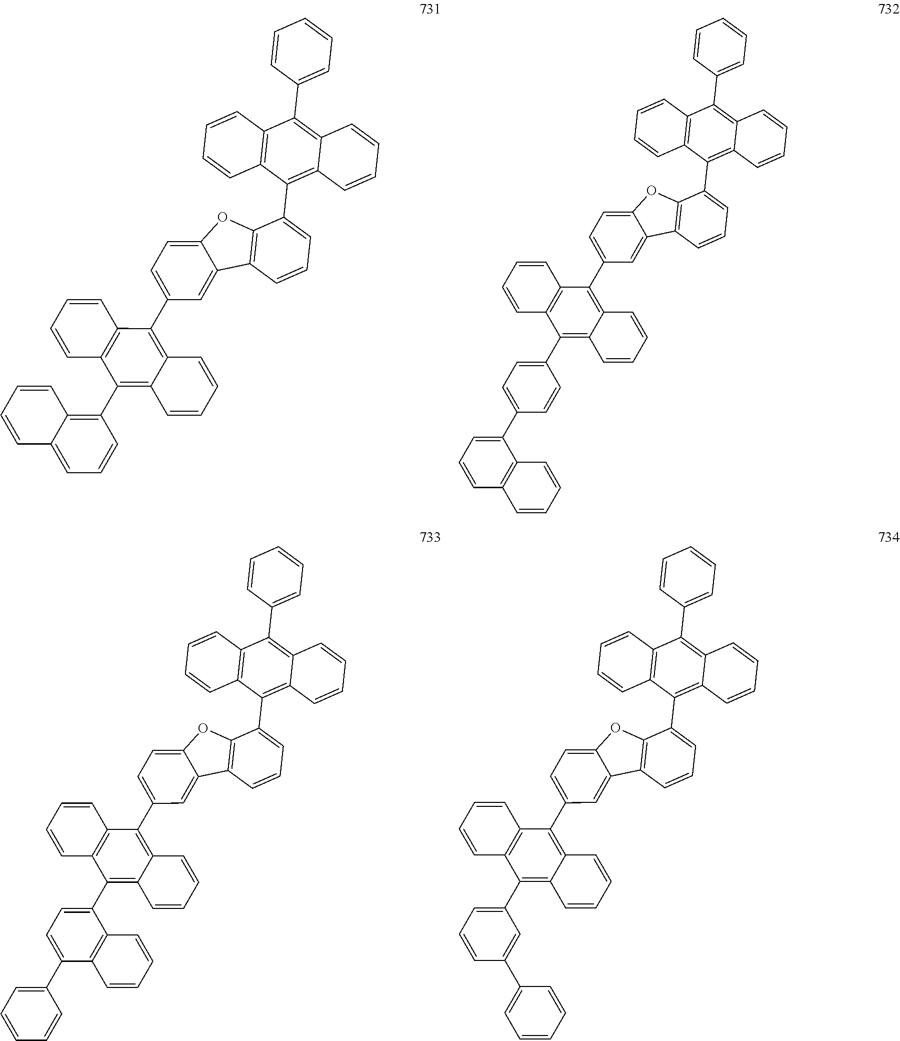 [SMILES: c1ccc(-c2c3ccccc3c(-c3cccc4c3oc3ccc(-c5c6ccccc6c(-c6cccc7ccccc67)c6ccccc56)cc34)c3ccccc23)cc1 ; c1ccc(-c2c3ccccc3c(-c3cccc4c3oc3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5oc6c(-c7c8ccccc8c(-c8ccccc8)c8ccccc78)cccc6c5c4)c4ccccc34)c3ccccc23)cc1 ; c1ccc(-c2cccc(-c3c4ccccc4c(-c4ccc5oc6c(-c7c8ccccc8c(-c8ccccc8)c8ccccc78)cccc6c5c4)c4ccccc34)c2)cc1]

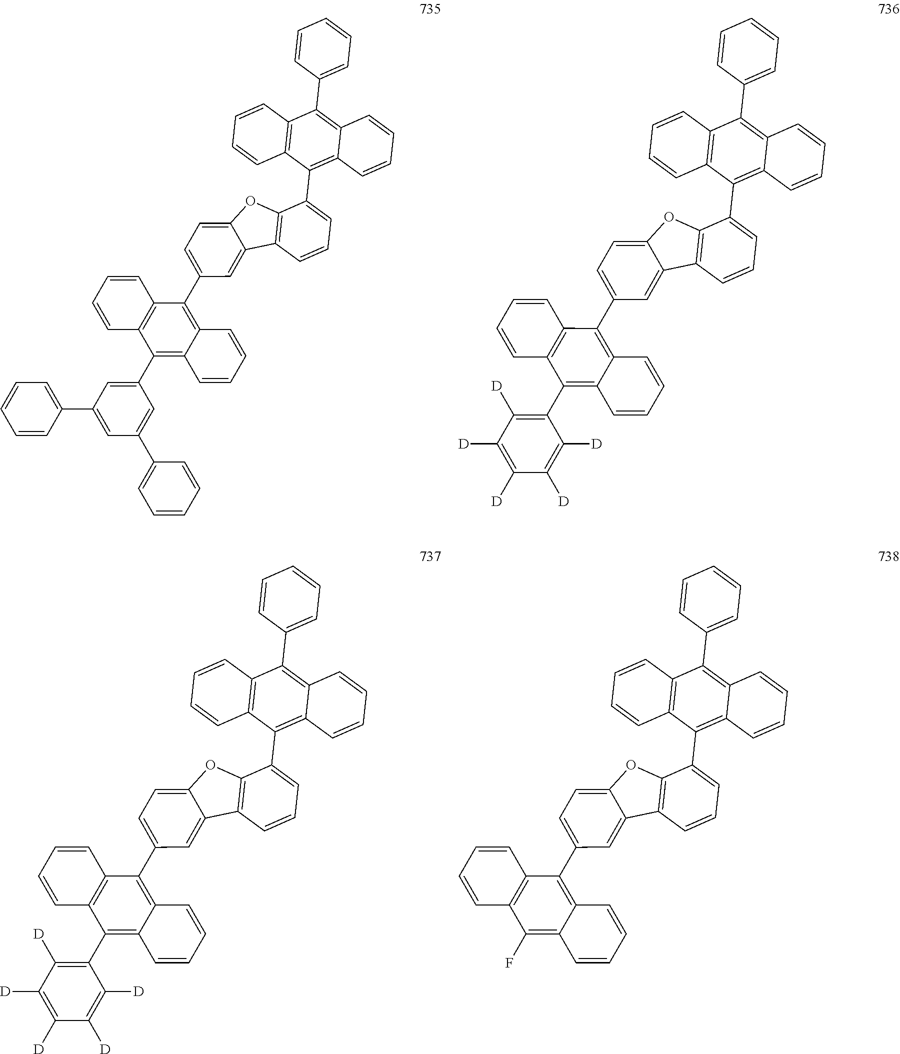 [SMILES: c1ccc(-c2cc(-c3ccccc3)cc(-c3c4ccccc4c(-c4ccc5oc6c(-c7c8ccccc8c(-c8ccccc8)c8ccccc78)cccc6c5c4)c4ccccc34)c2)cc1 ; [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H] ; [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H] ; Fc1c2ccccc2c(-c2ccc3oc4c(-c5c6ccccc6c(-c6ccccc6)c6ccccc56)cccc4c3c2)c2ccccc12]

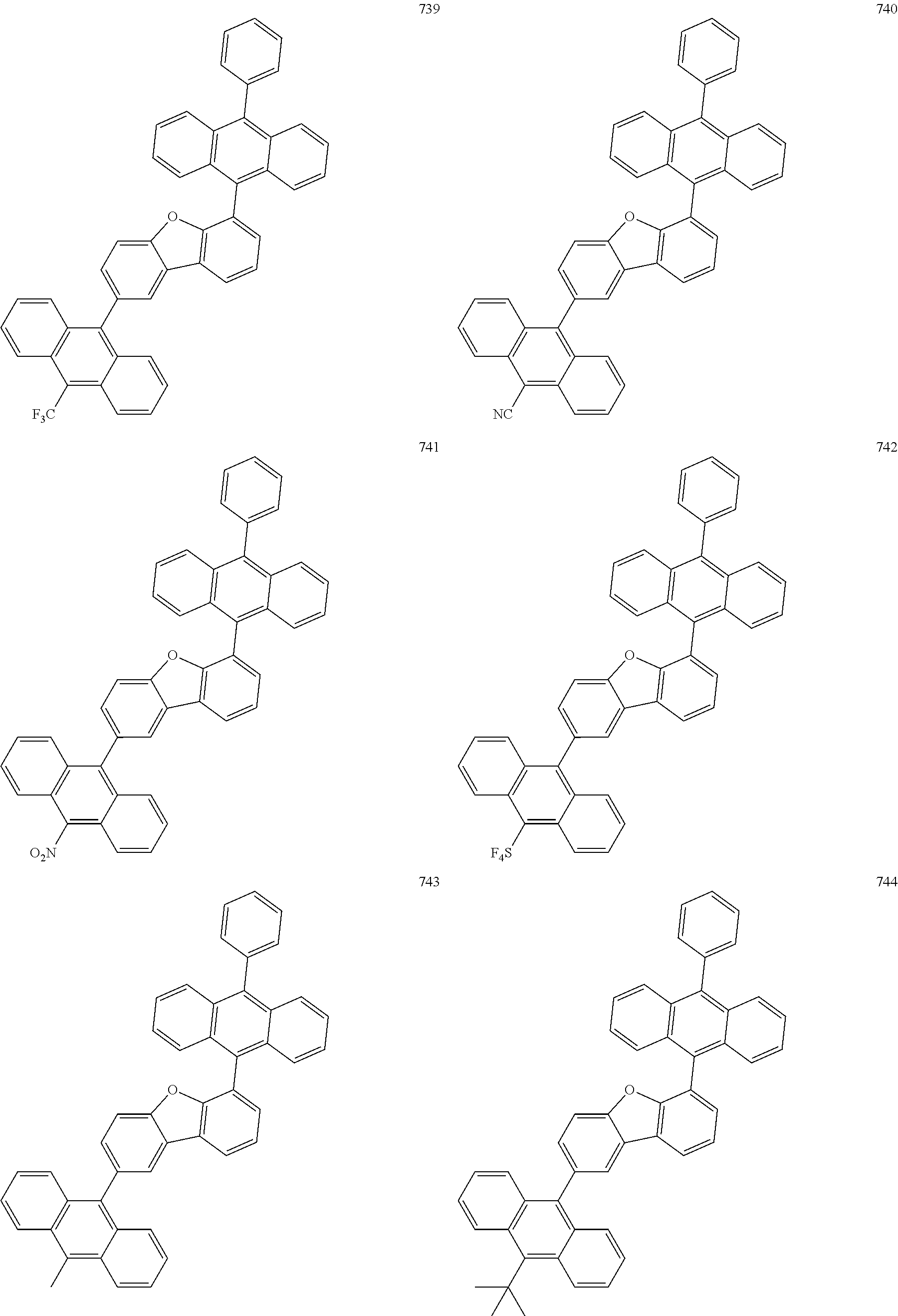

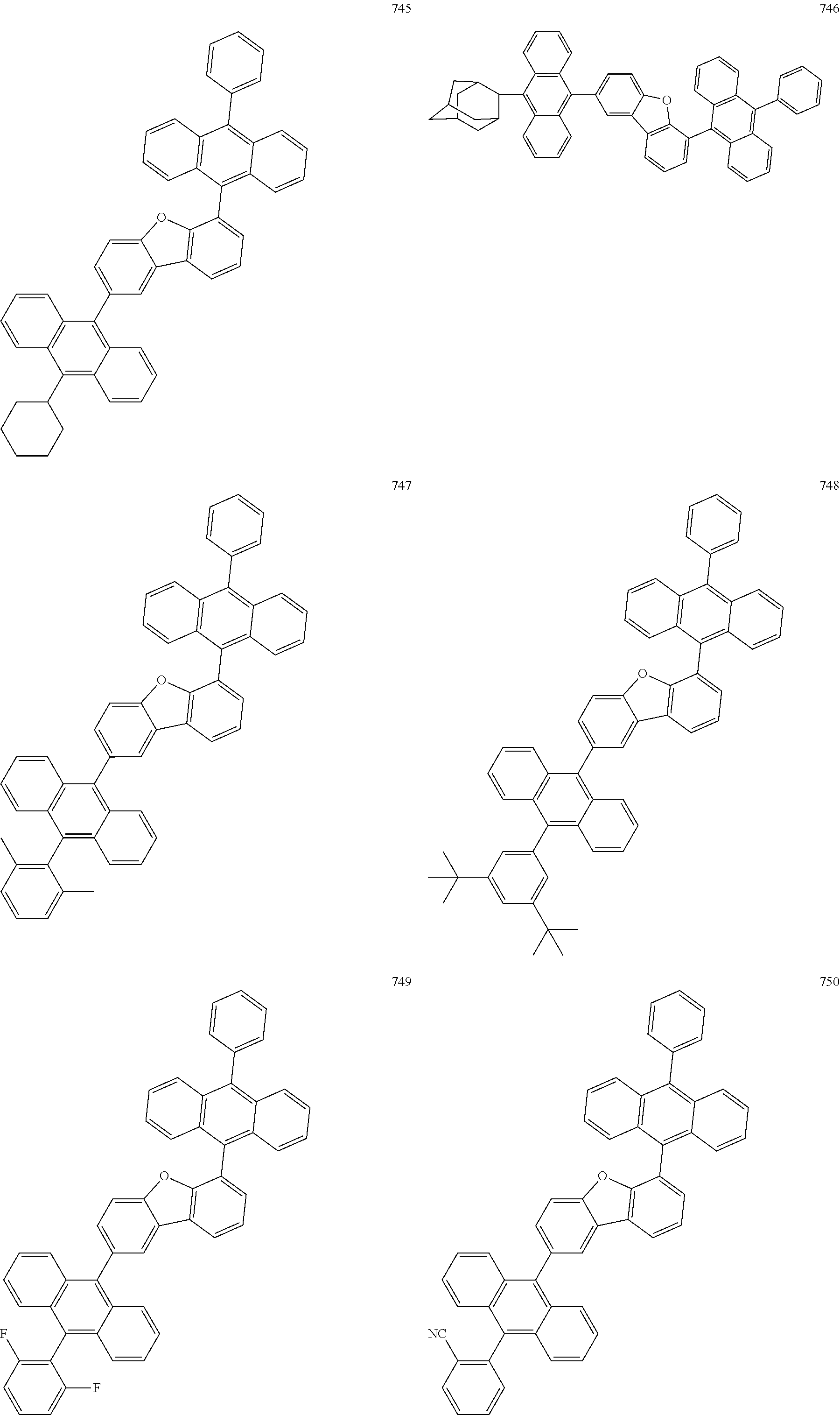

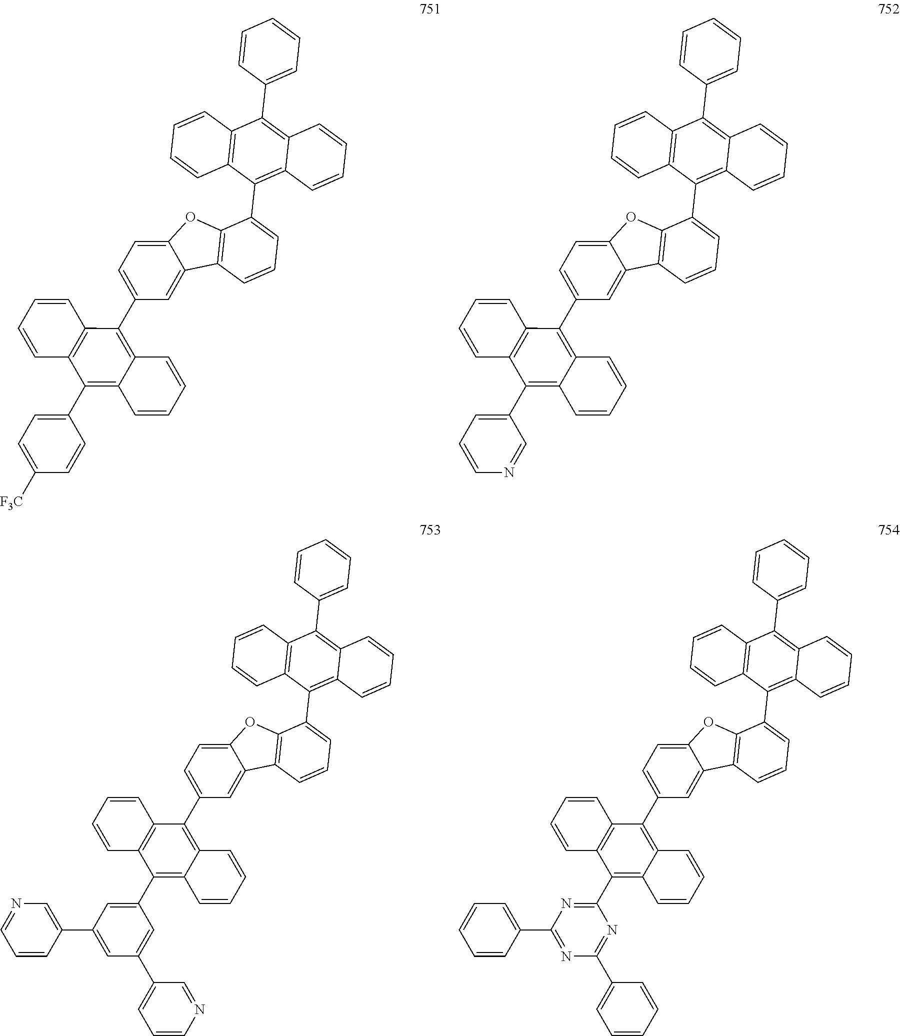 [SMILES: FC(F)(F)c1ccc(-c2c3ccccc3c(-c3ccc4oc5c(-c6c7ccccc7c(-c7ccccc7)c7ccccc67)cccc5c4c3)c3ccccc23)cc1 ; c1ccc(-c2c3ccccc3c(-c3cccc4c3oc3ccc(-c5c6ccccc6c(-c6cccnc6)c6ccccc56)cc34)c3ccccc23)cc1 ; c1ccc(-c2c3ccccc3c(-c3cccc4c3oc3ccc(-c5c6ccccc6c(-c6cc(-c7cccnc7)cc(-c7cccnc7)c6)c6ccccc56)cc34)c3ccccc23)cc1 ; c1ccc(-c2nc(-c3ccccc3)nc(-c3c4ccccc4c(-c4ccc5oc6c(-c7c8ccccc8c(-c8ccccc8)c8ccccc78)cccc6c5c4)c4ccccc34)n2)cc1]

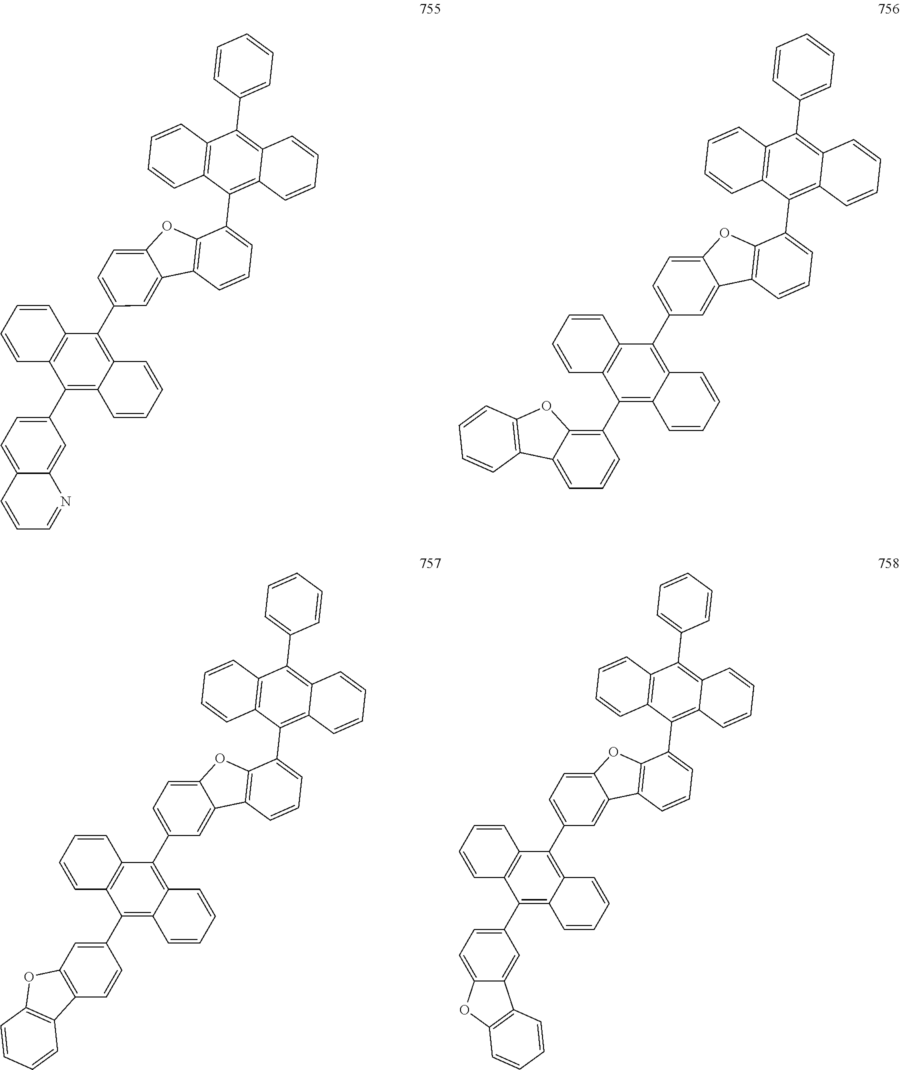 [SMILES: c1ccc(-c2c3ccccc3c(-c3cccc4c3oc3ccc(-c5c6ccccc6c(-c6ccc7cccnc7c6)c6ccccc56)cc34)c3ccccc23)cc1 ; c1ccc(-c2c3ccccc3c(-c3cccc4c3oc3ccc(-c5c6ccccc6c(-c6cccc7c6oc6ccccc67)c6ccccc56)cc34)c3ccccc23)cc1 ; c1ccc(-c2c3ccccc3c(-c3cccc4c3oc3ccc(-c5c6ccccc6c(-c6ccc7c(c6)oc6ccccc67)c6ccccc56)cc34)c3ccccc23)cc1 ; c1ccc(-c2c3ccccc3c(-c3cccc4c3oc3ccc(-c5c6ccccc6c(-c6ccc7oc8ccccc8c7c6)c6ccccc56)cc34)c3ccccc23)cc1]

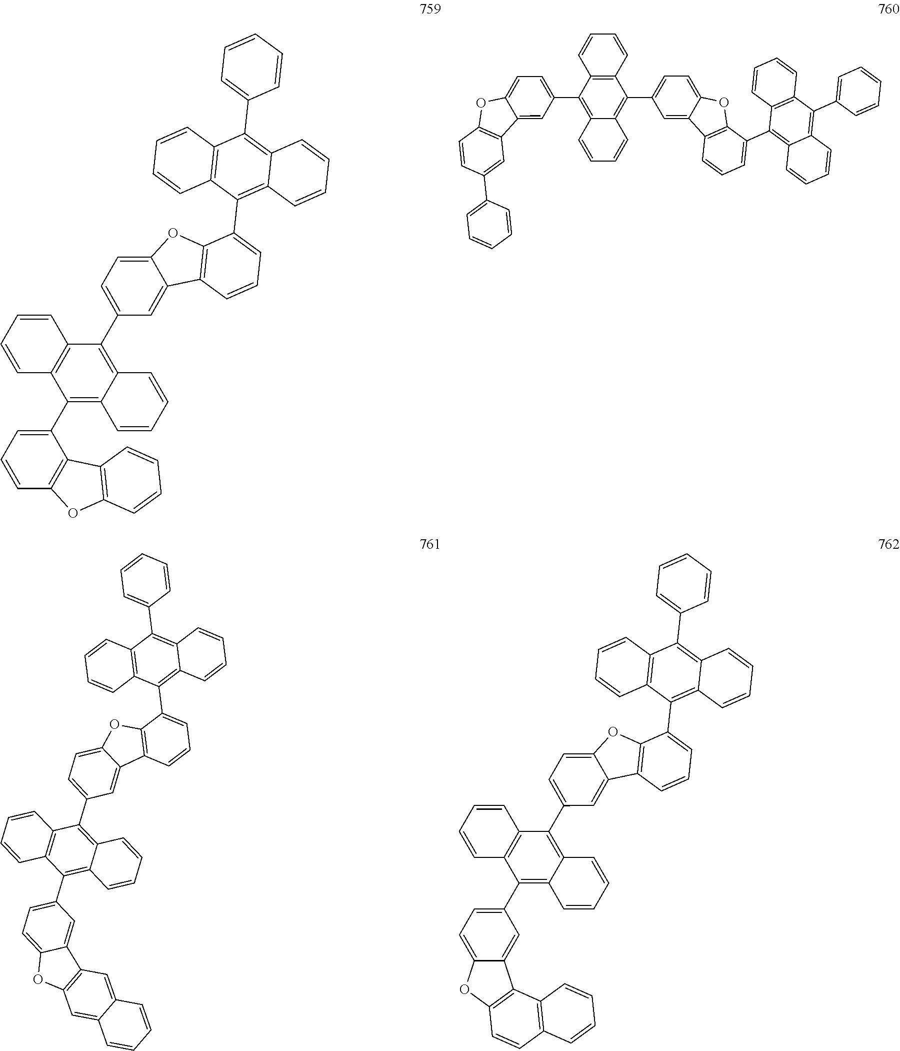 [SMILES: c1ccc(-c2c3ccccc3c(-c3cccc4c3oc3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3oc3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3cccc4c3oc3ccc(-c5c6ccccc6c(-c6ccc7oc8ccc9ccccc9c8c7c6)c6ccccc56)cc34)c3ccccc23)cc1]

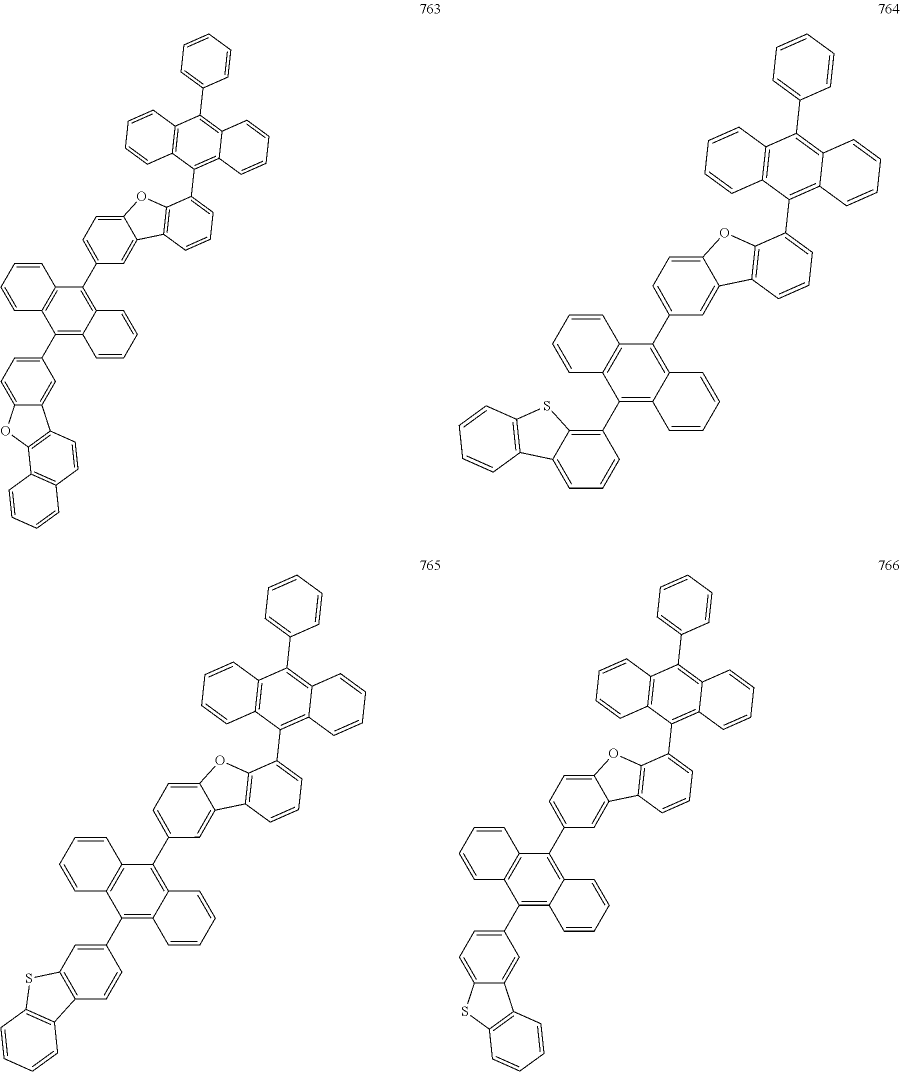 [SMILES: c1ccc(-c2c3ccccc3c(-c3cccc4c3oc3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3cccc4c3oc3ccc(-c5c6ccccc6c(-c6cccc7c6sc6ccccc67)c6ccccc56)cc34)c3ccccc23)cc1 ; c1ccc(-c2c3ccccc3c(-c3cccc4c3oc3ccc(-c5c6ccccc6c(-c6ccc7c(c6)sc6ccccc67)c6ccccc56)cc34)c3ccccc23)cc1 ; c1ccc(-c2c3ccccc3c(-c3cccc4c3oc3ccc(-c5c6ccccc6c(-c6ccc7sc8ccccc8c7c6)c6ccccc56)cc34)c3ccccc23)cc1]

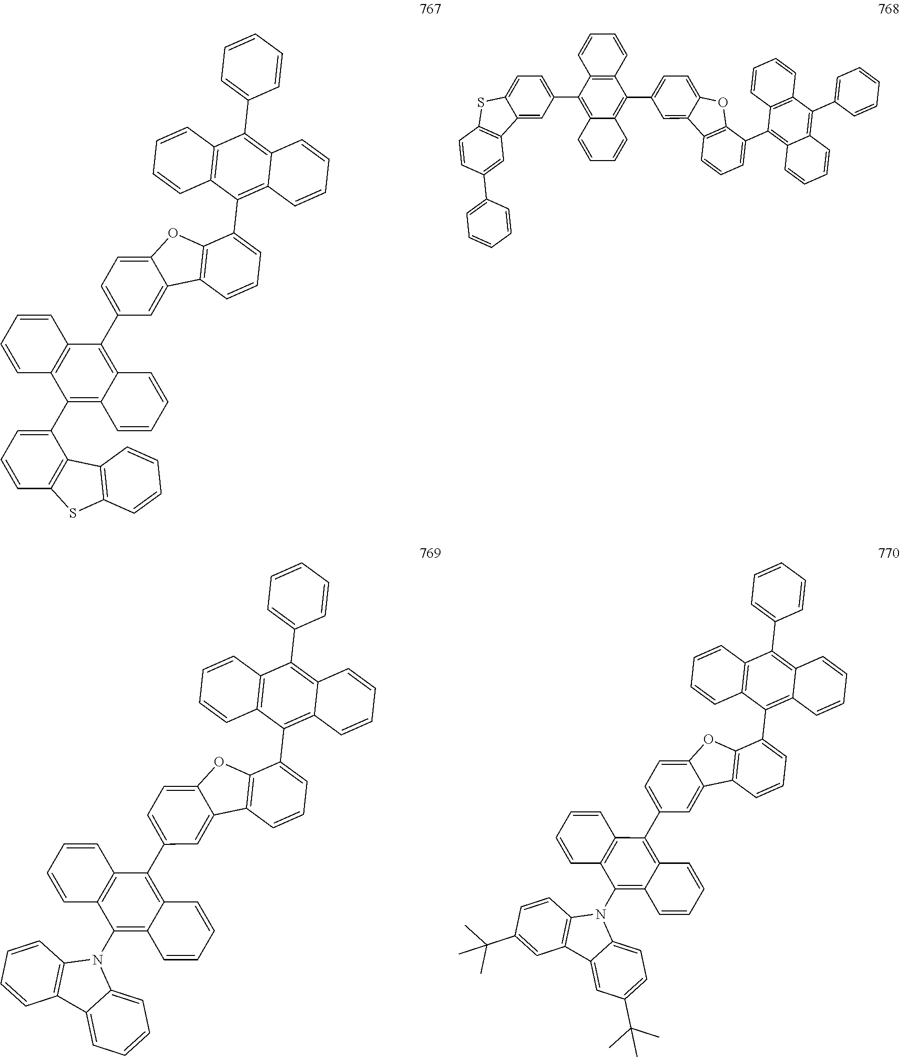 [SMILES: c1ccc(-c2c3ccccc3c(-c3cccc4c3oc3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3oc3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3oc4c(-c5c6ccccc6c(-c6ccccc6)c6ccccc56)cccc4c3c2)c2ccccc12]

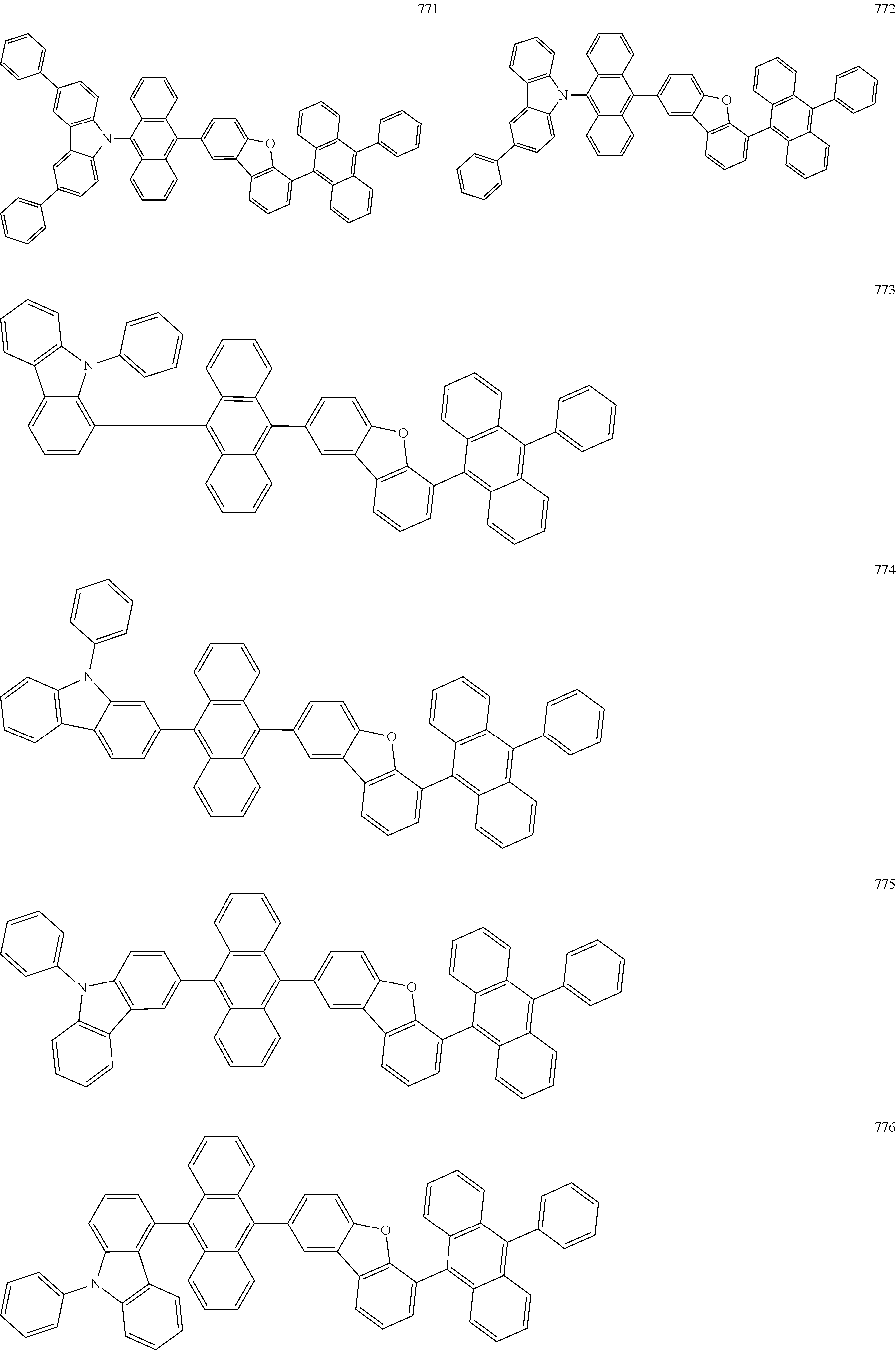

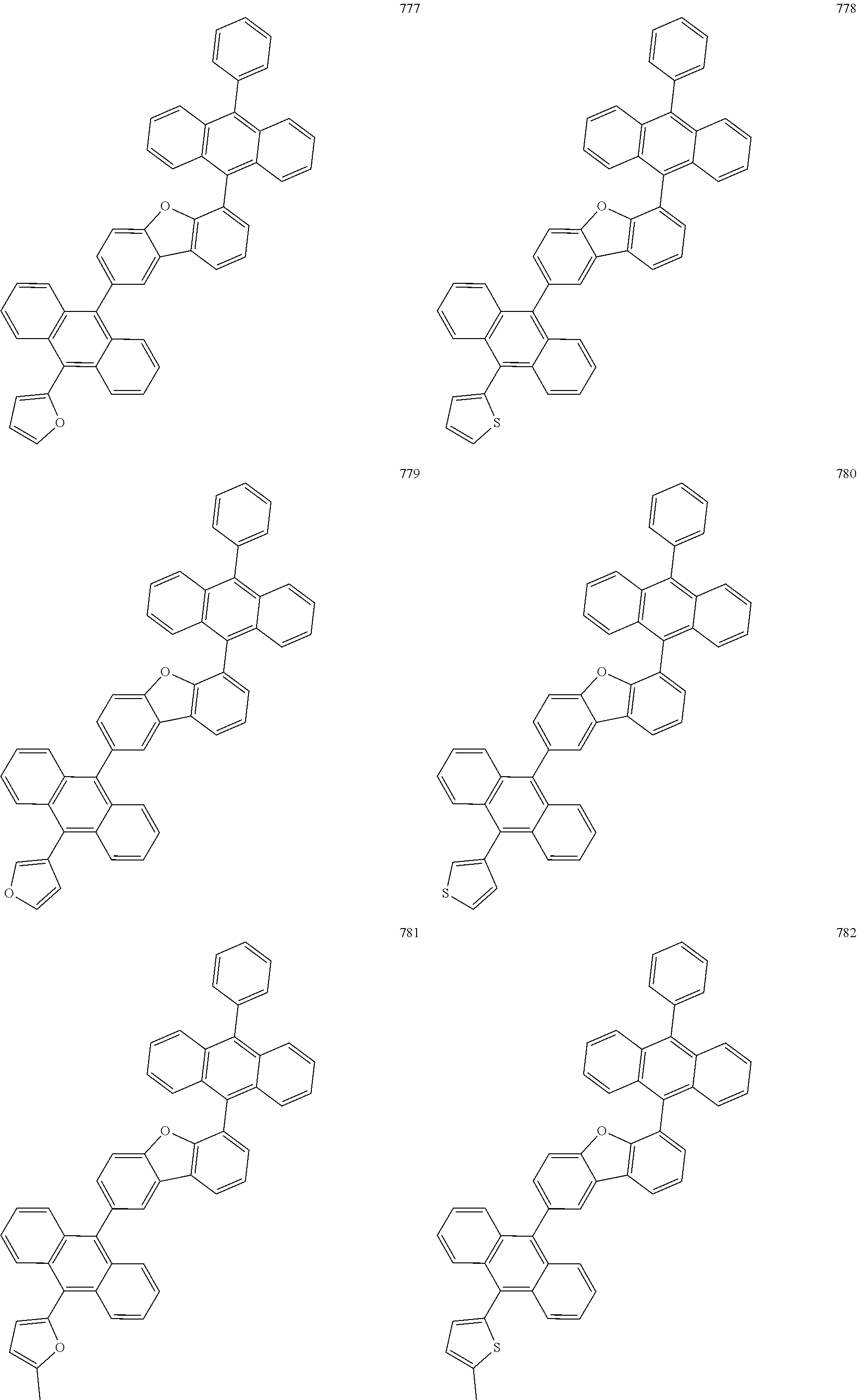

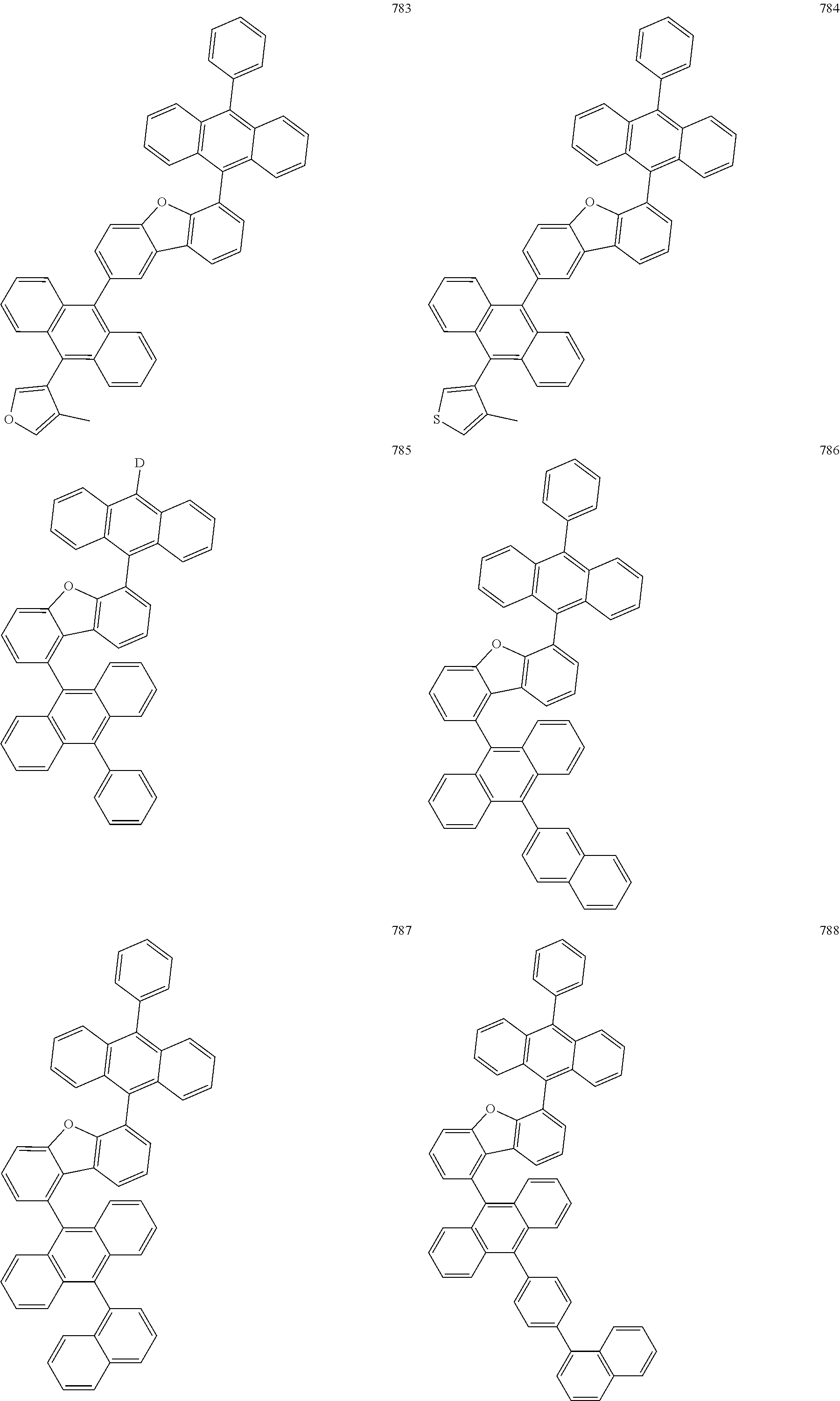

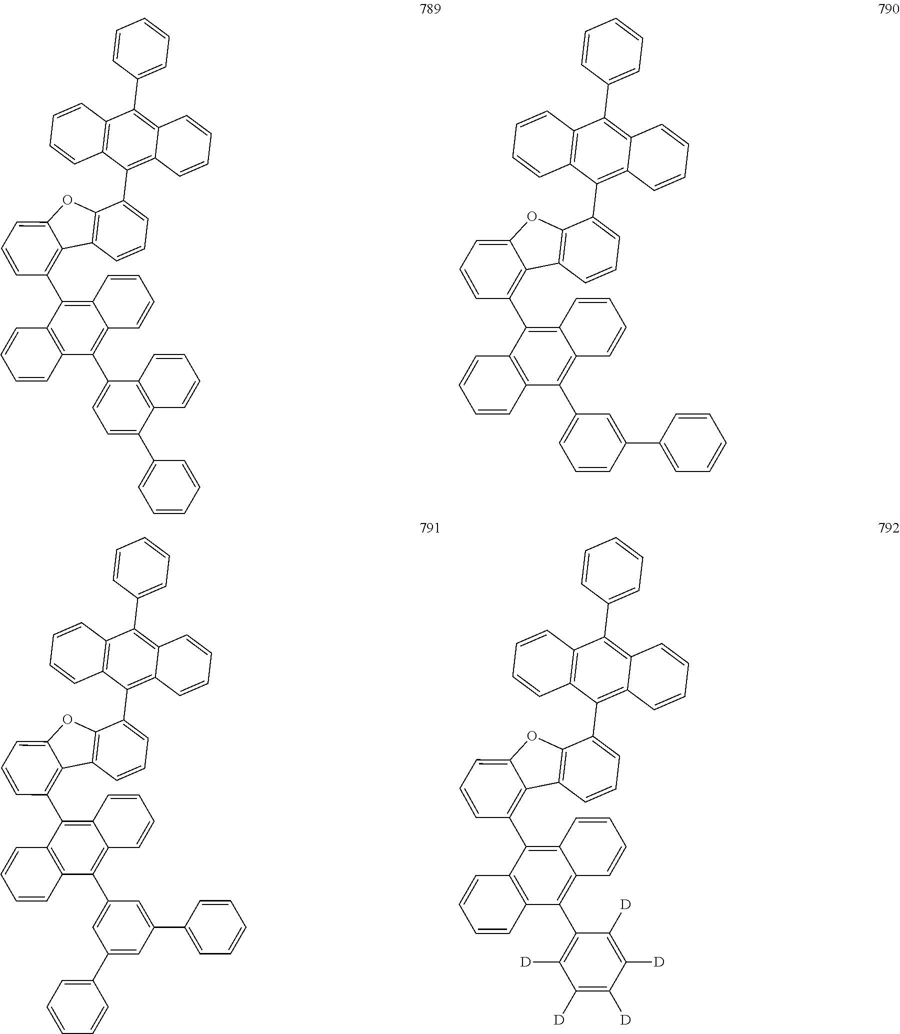

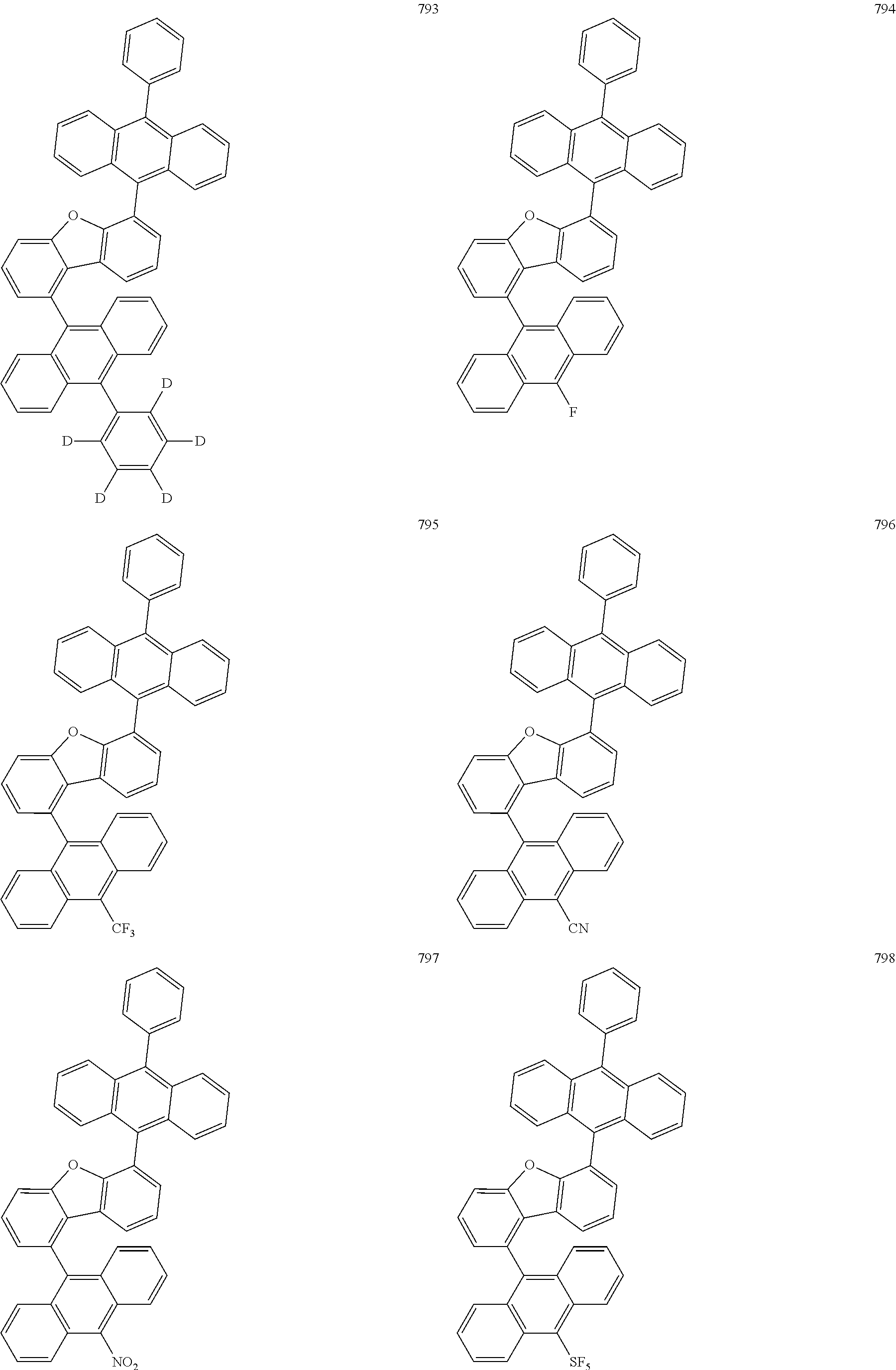

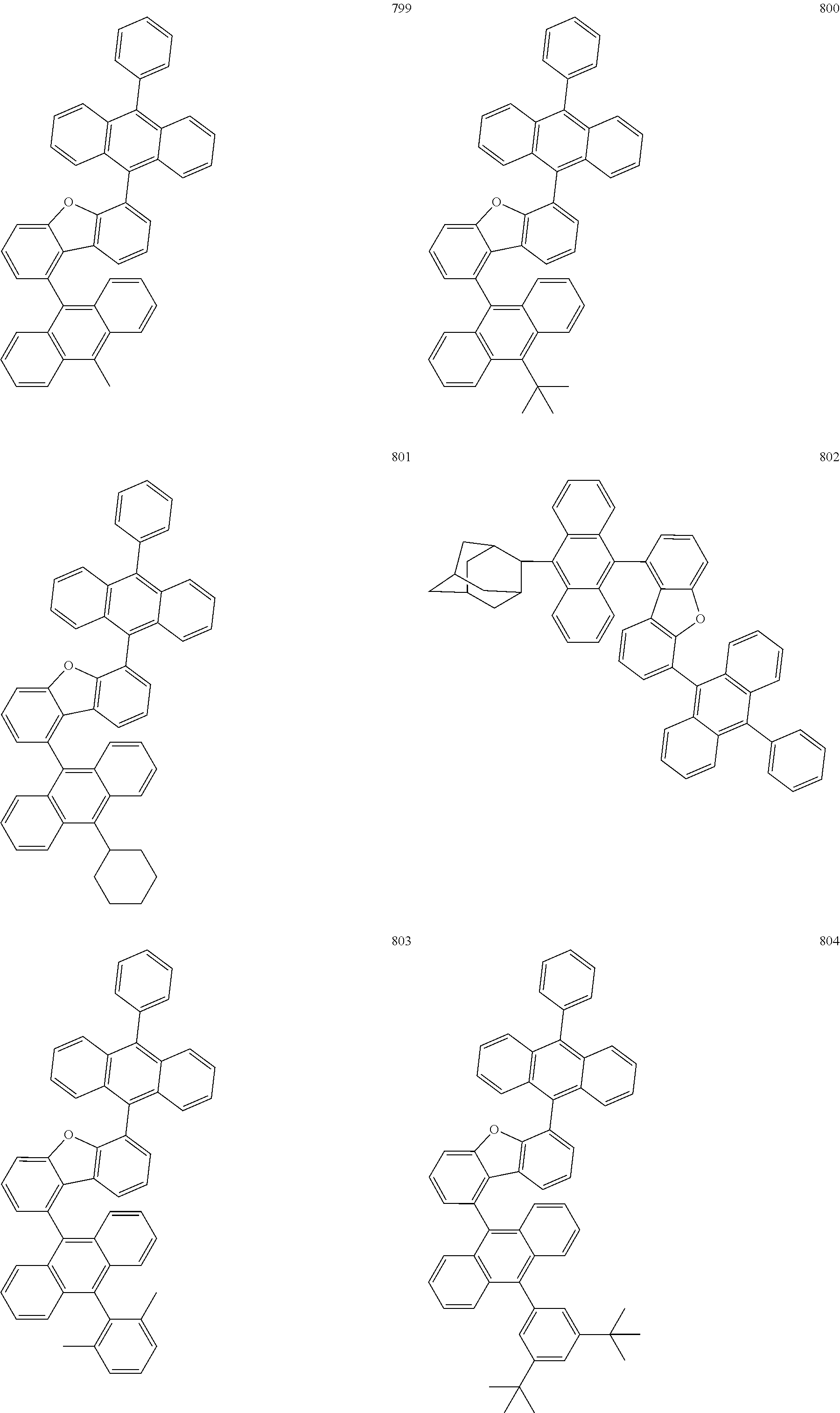

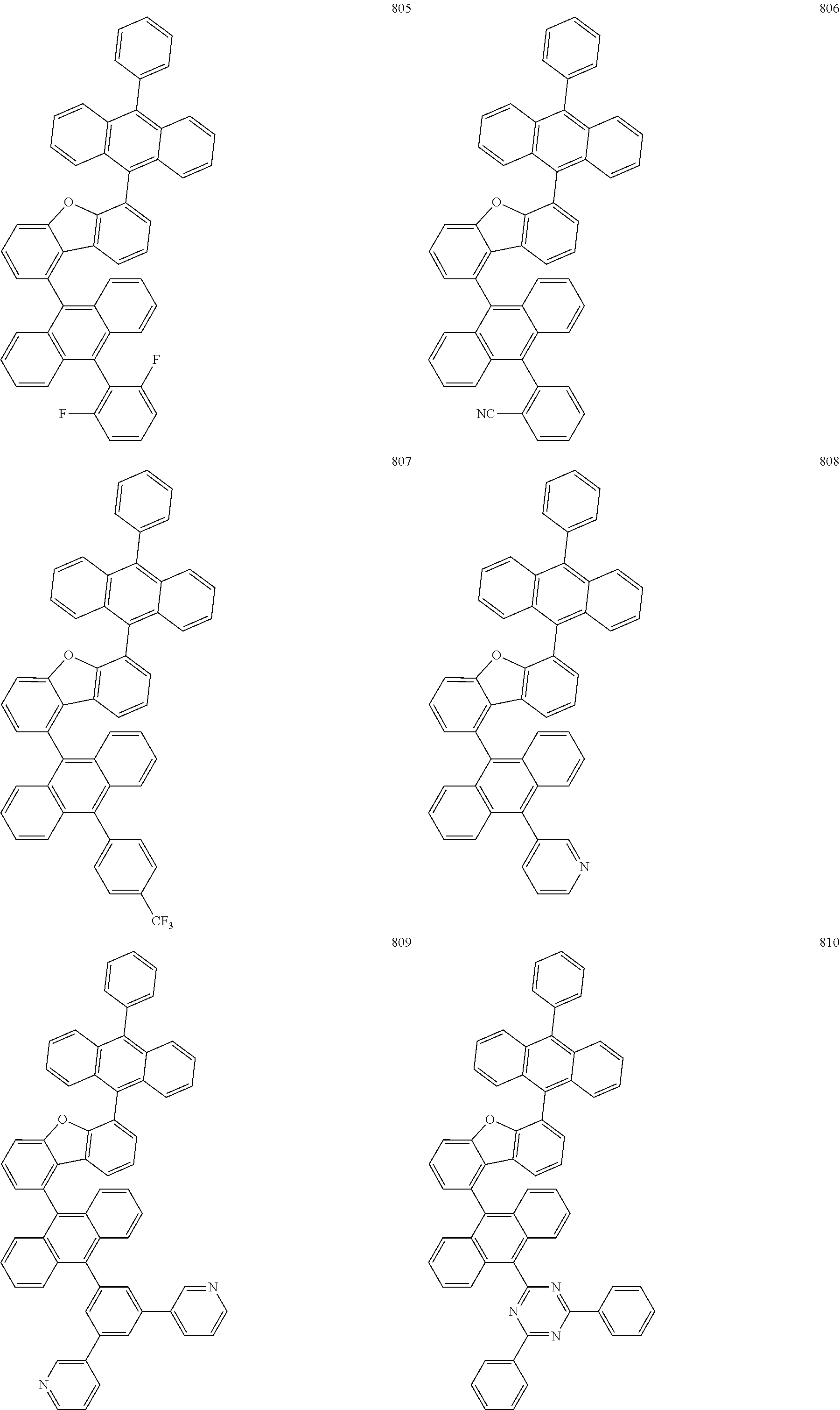

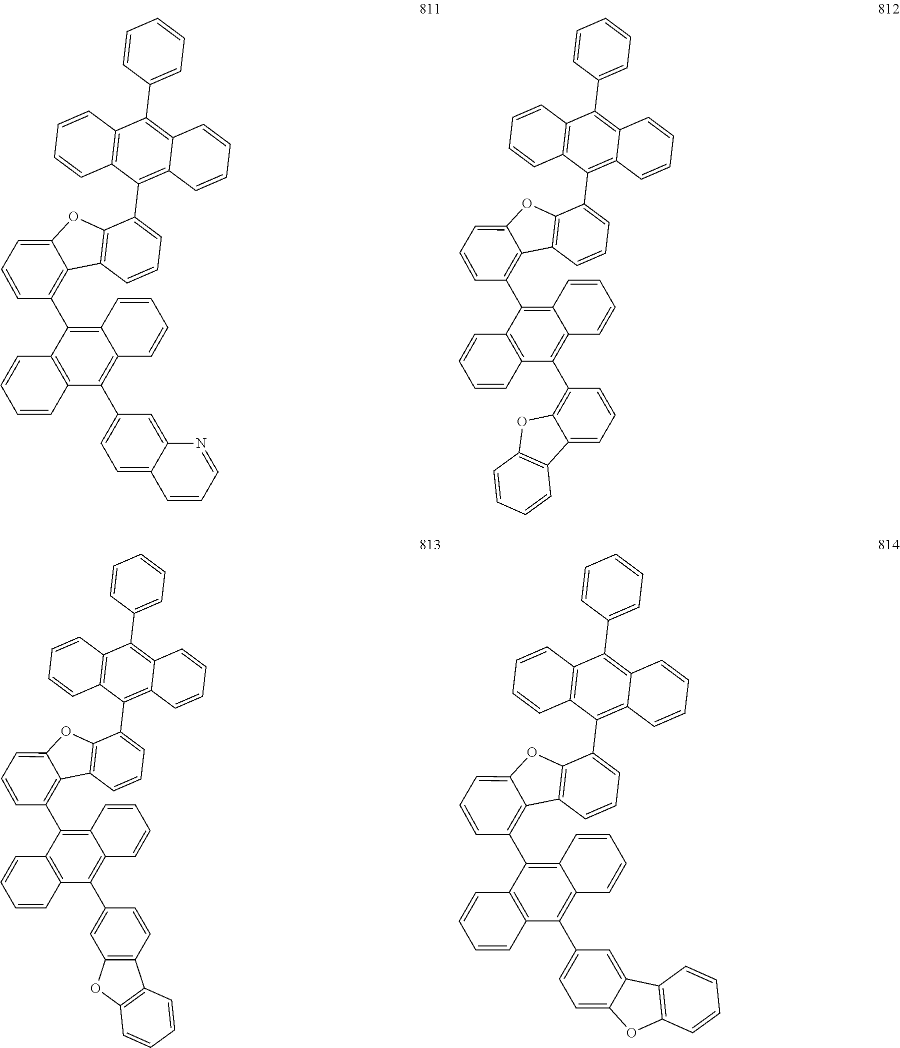 [SMILES: c1ccc(-c2c3ccccc3c(-c3cccc4c3oc3cccc(-c5c6ccccc6c(-c6ccc7cccnc7c6)c6ccccc56)c34)c3ccccc23)cc1 ; c1ccc(-c2c3ccccc3c(-c3cccc4c3oc3cccc(-c5c6ccccc6c(-c6cccc7c6oc6ccccc67)c6ccccc56)c34)c3ccccc23)cc1 ; c1ccc(-c2c3ccccc3c(-c3cccc4c3oc3cccc(-c5c6ccccc6c(-c6ccc7c(c6)oc6ccccc67)c6ccccc56)c34)c3ccccc23)cc1 ; c1ccc(-c2c3ccccc3c(-c3cccc4c3oc3cccc(-c5c6ccccc6c(-c6ccc7oc8ccccc8c7c6)c6ccccc56)c34)c3ccccc23)cc1]

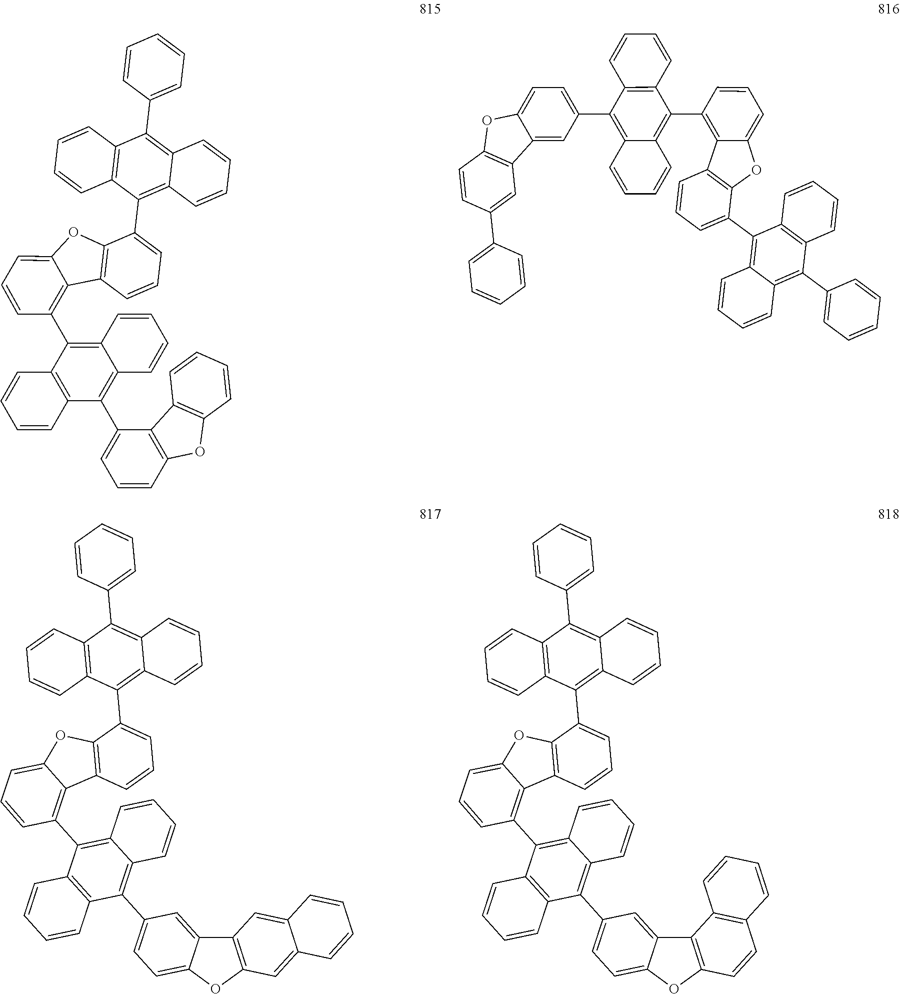

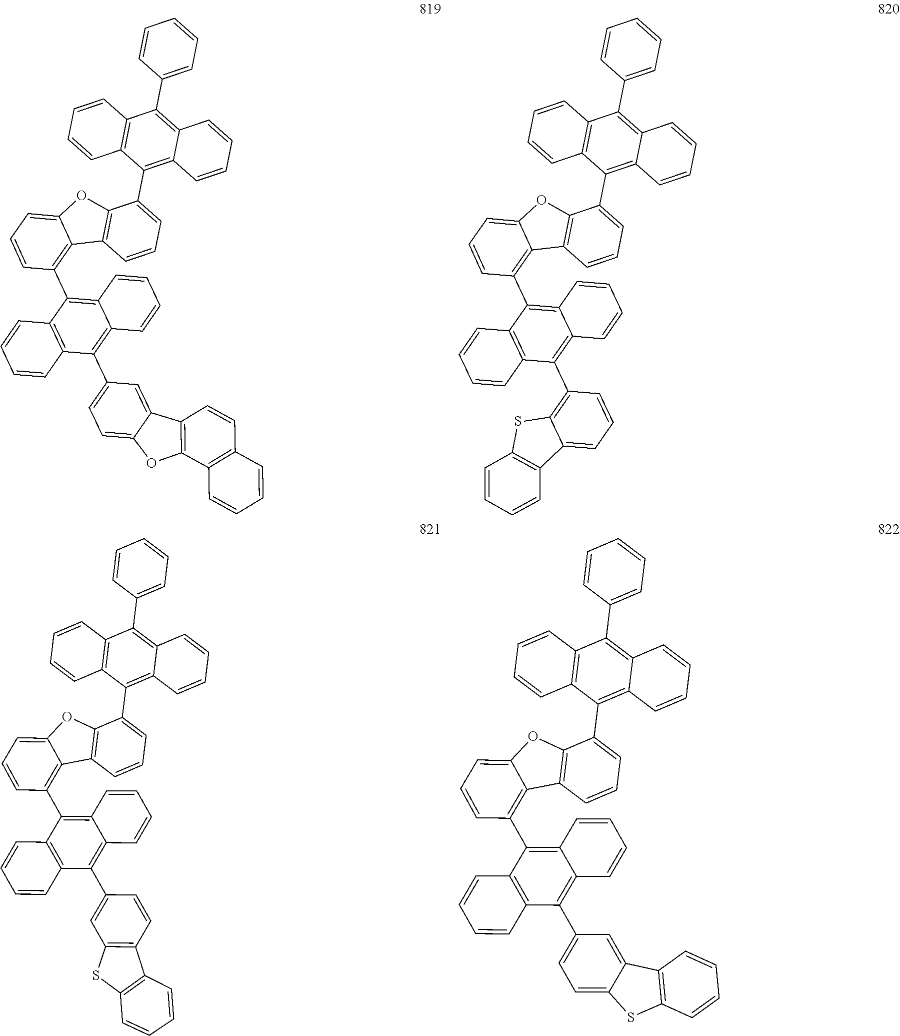 [SMILES: c1ccc(-c2c3ccccc3c(-c3cccc4c3oc3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3oc3cccc(-c5c6ccccc6c(-c6cccc7c6sc6ccccc67)c6ccccc56)c34)c3ccccc23)cc1 ; c1ccc(-c2c3ccccc3c(-c3cccc4c3oc3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3oc3cccc(-c5c6ccccc6c(-c6ccc7sc8ccccc8c7c6)c6ccccc56)c34)c3ccccc23)cc1]

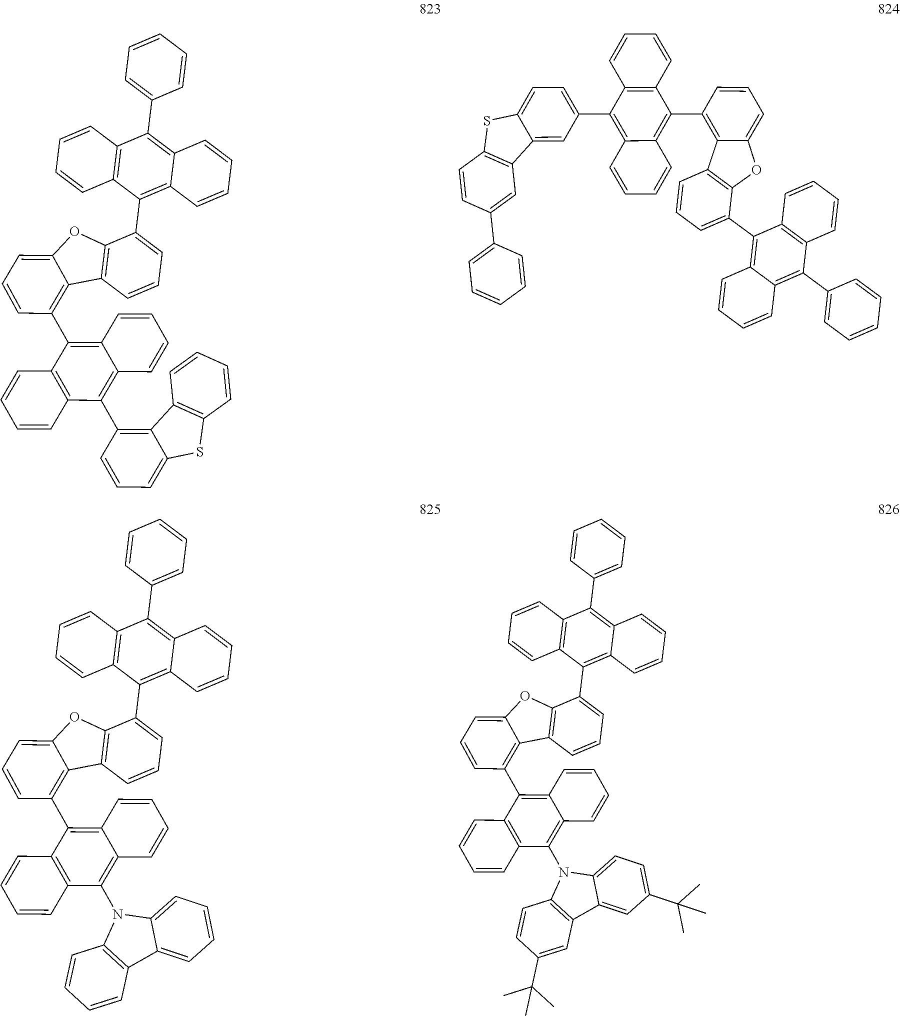

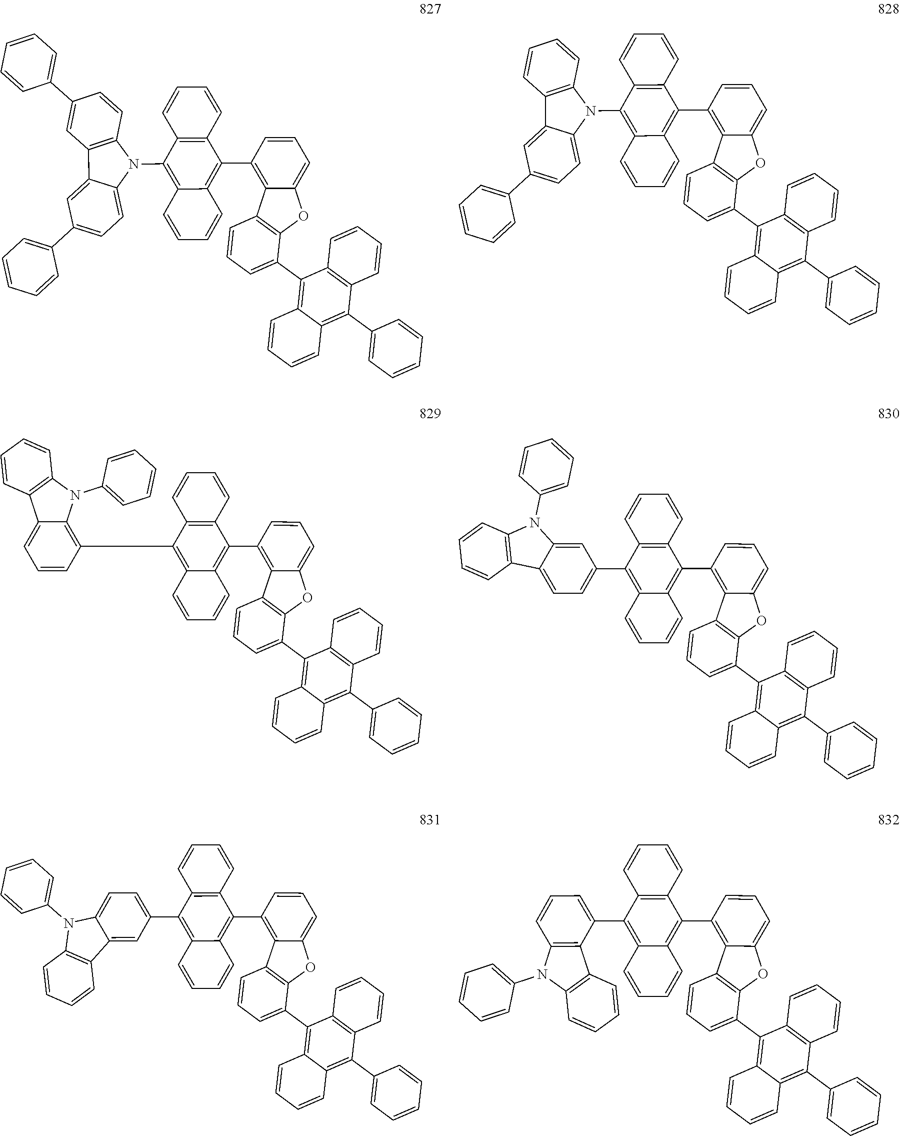

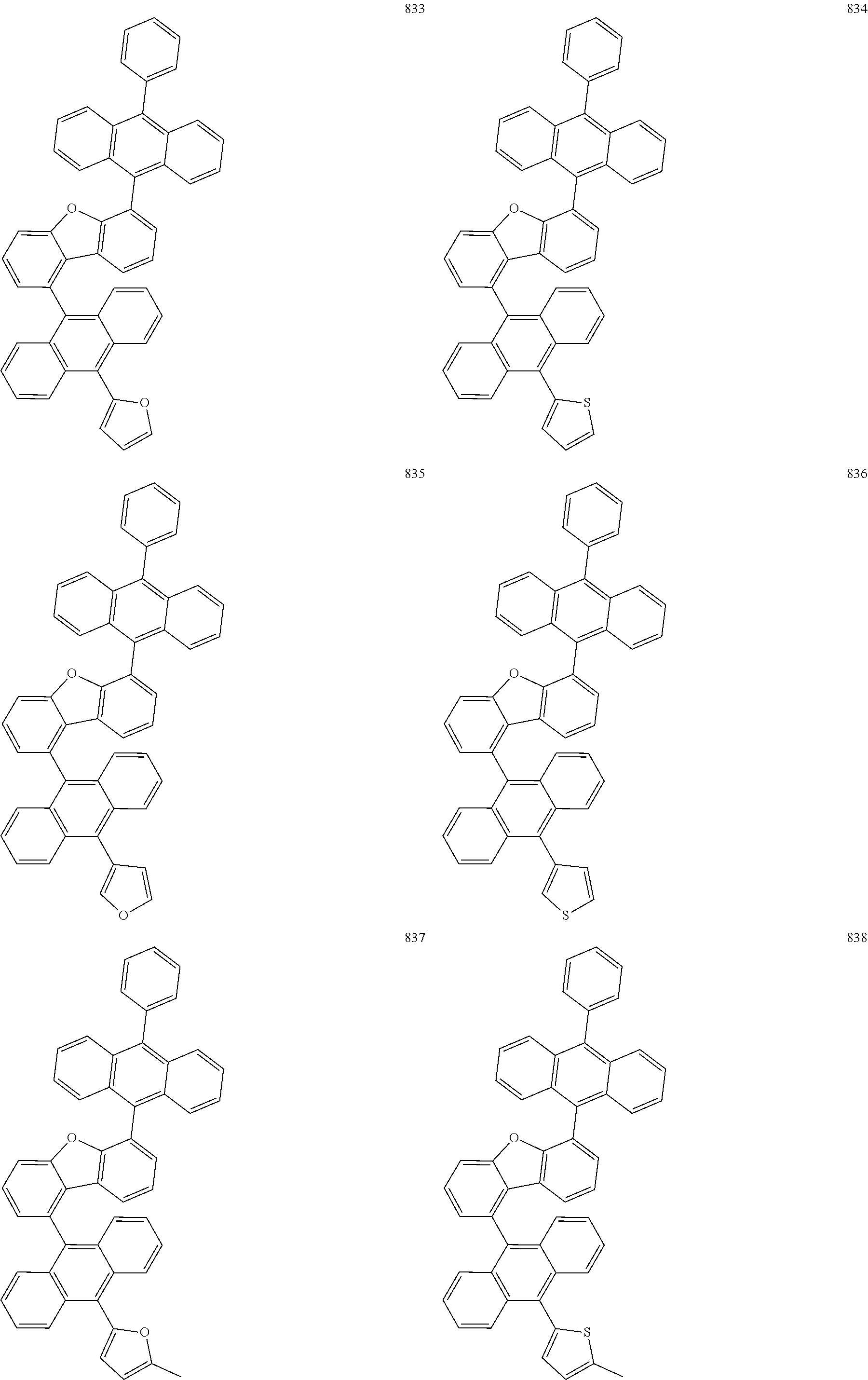

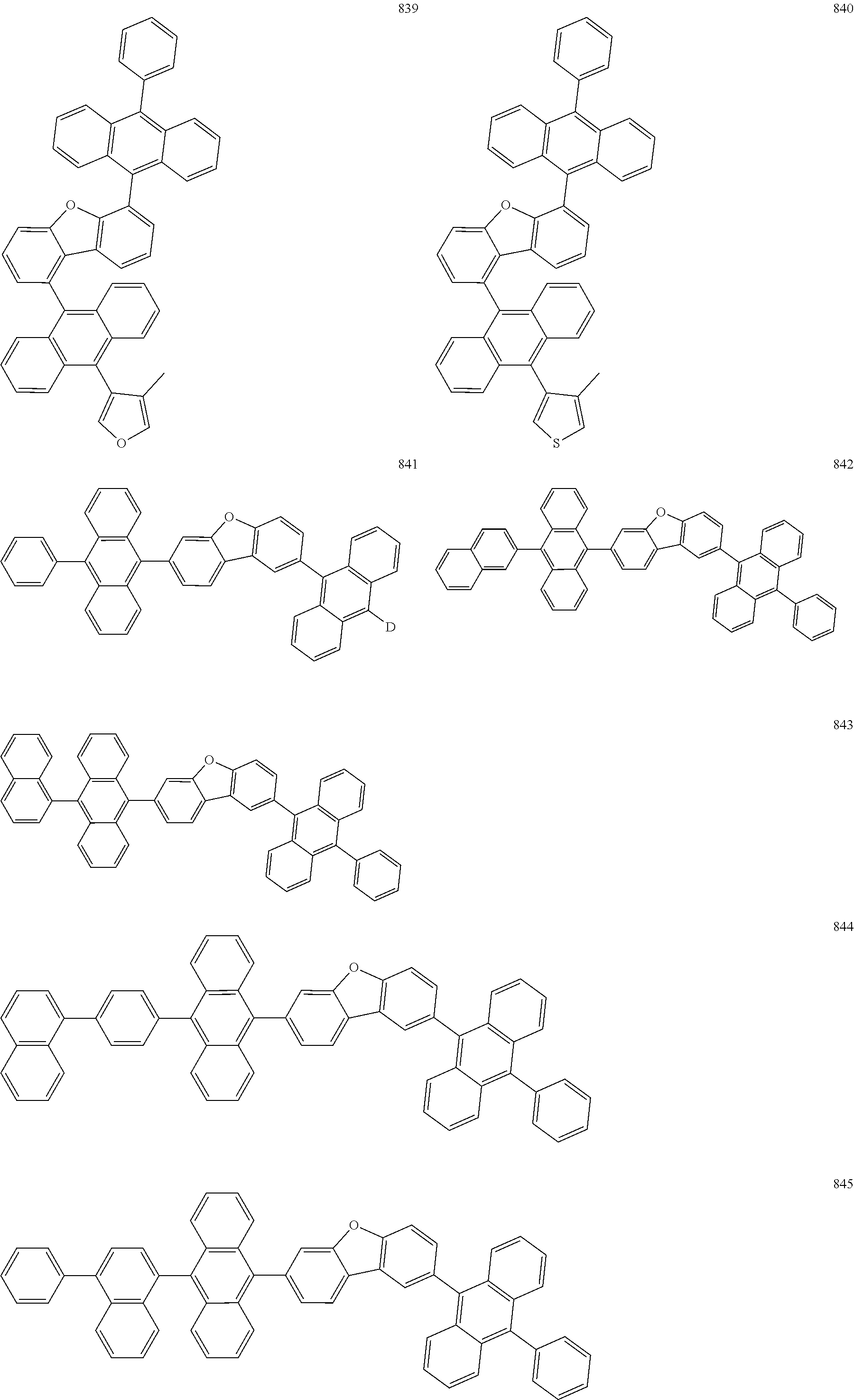

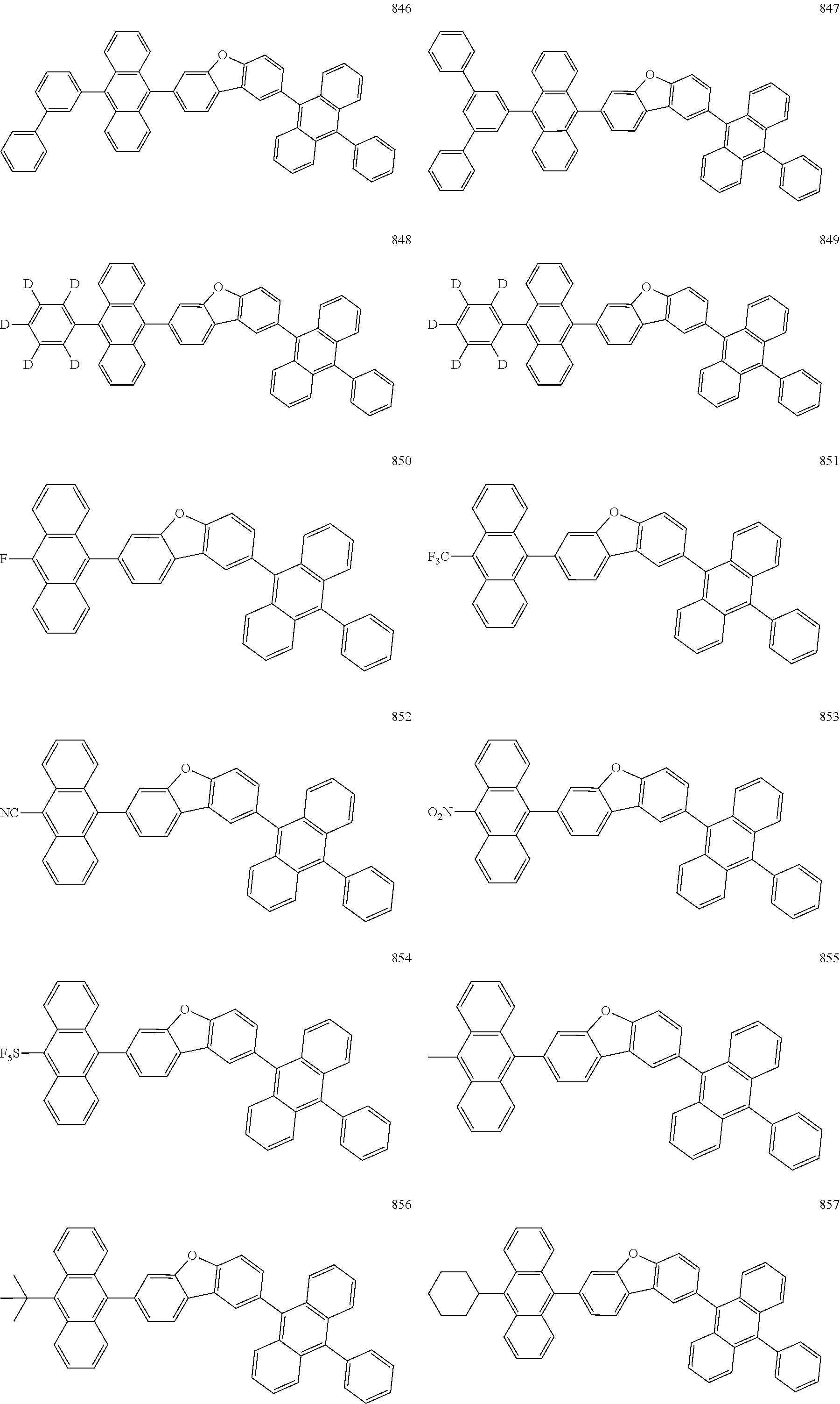

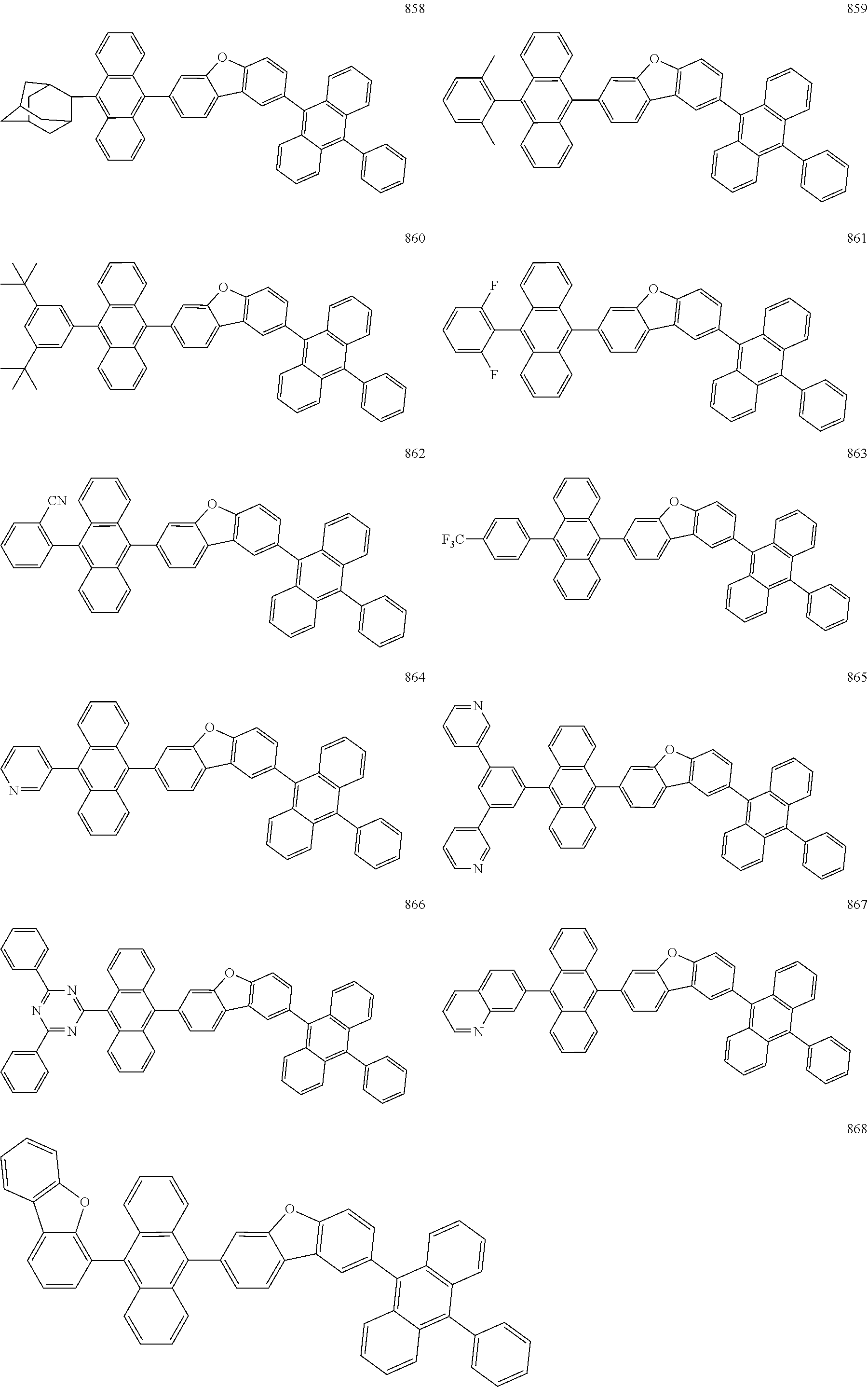

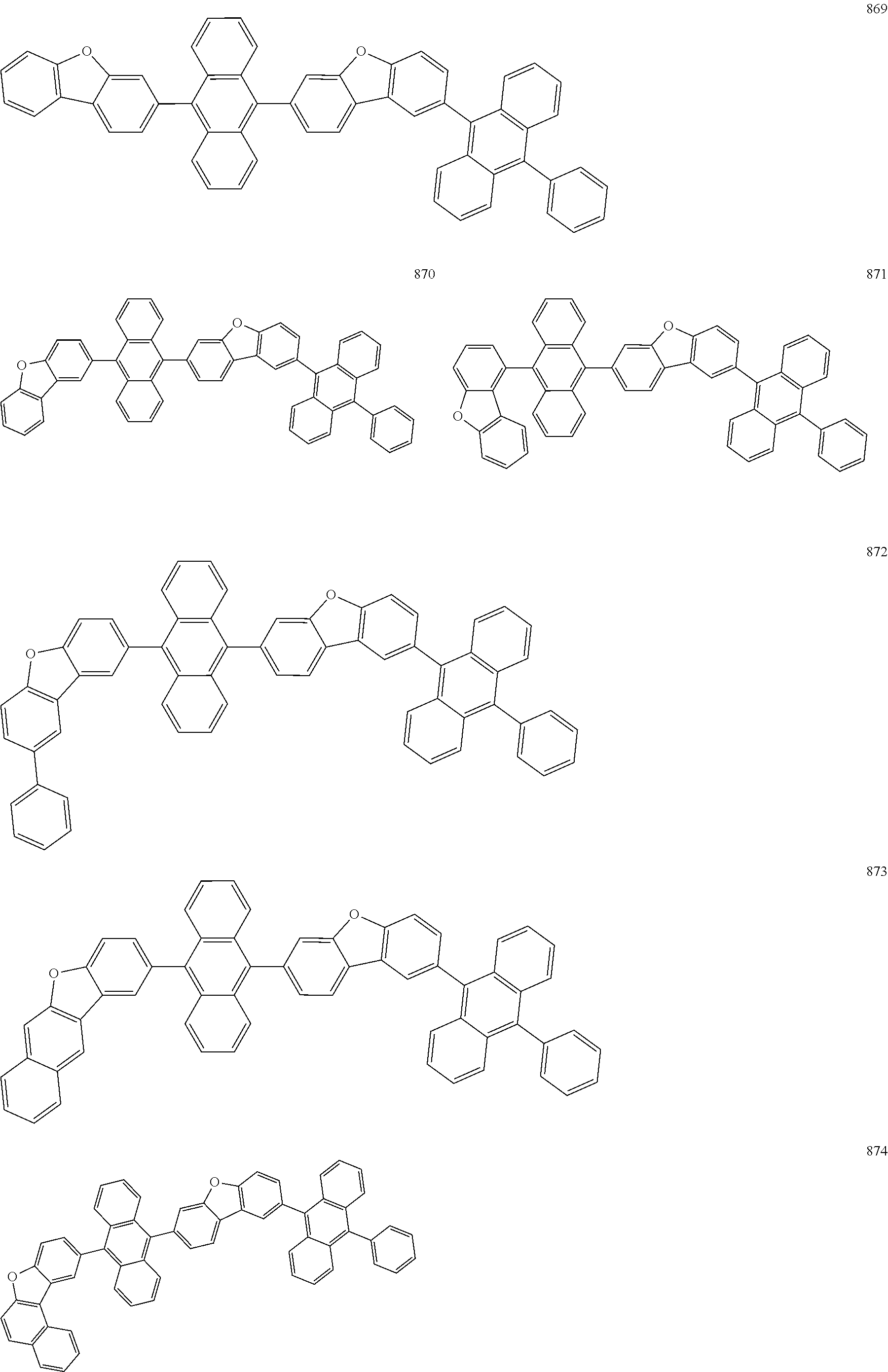

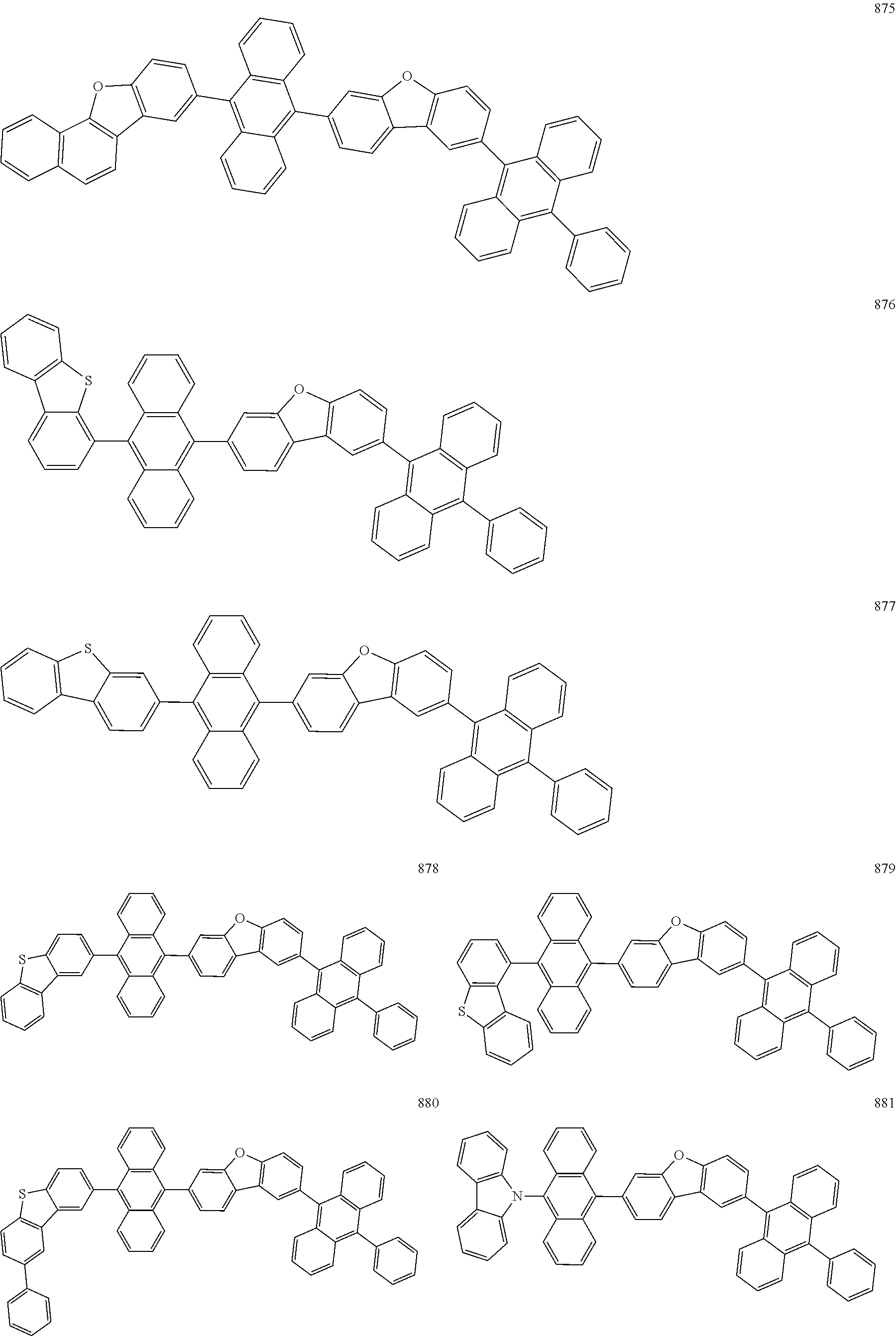

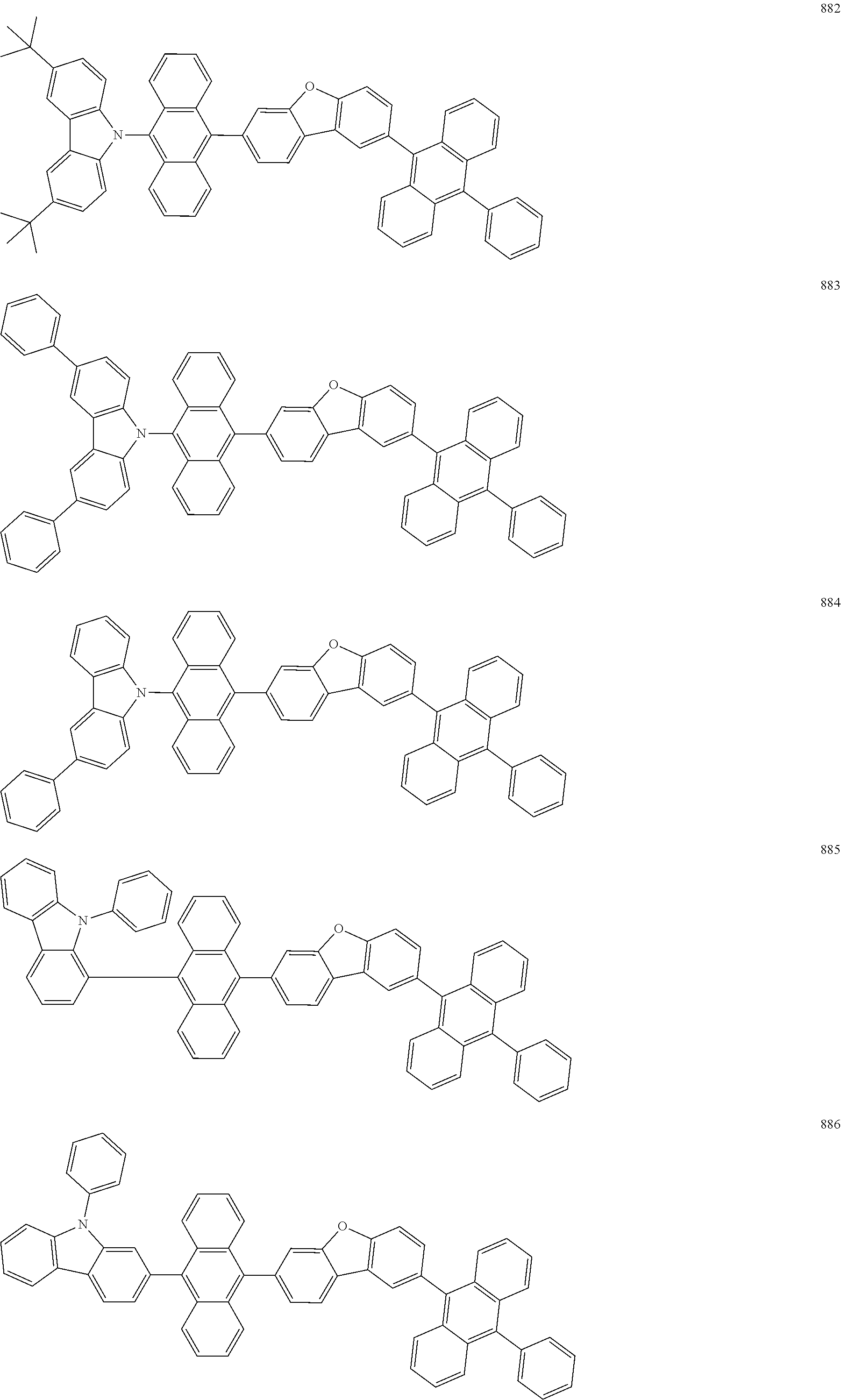

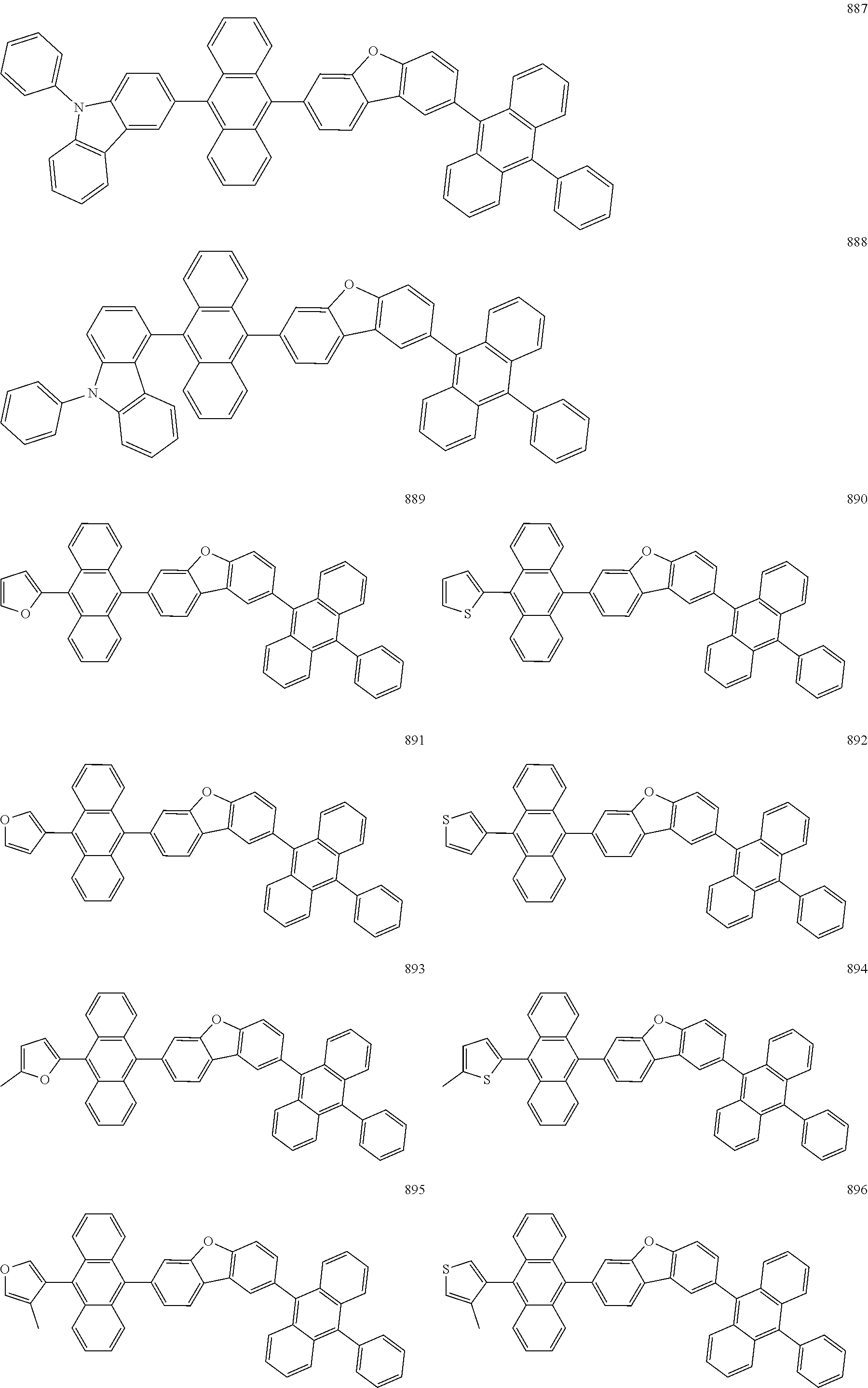

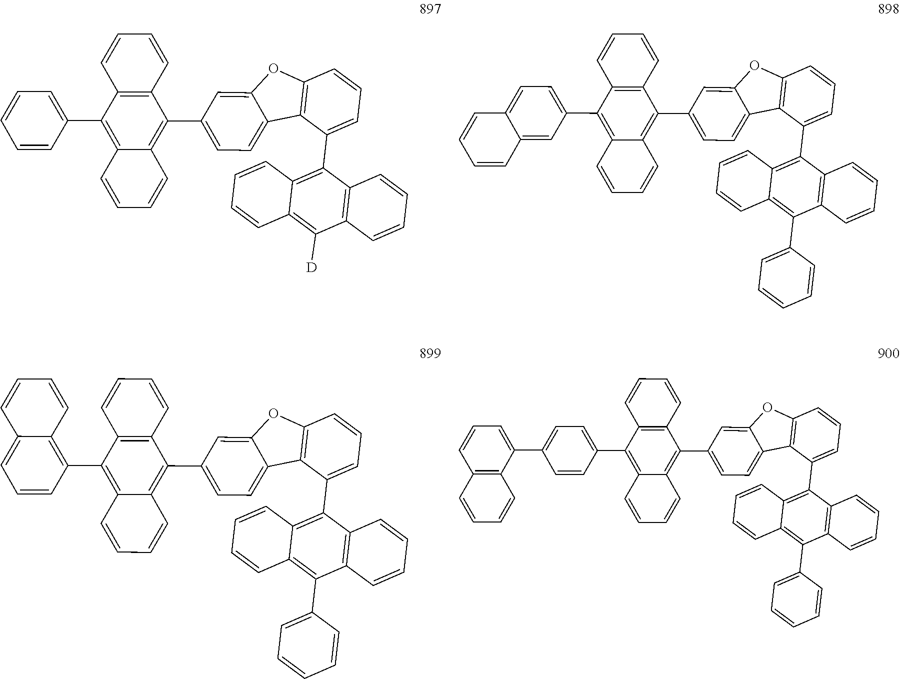 [SMILES: [2H]c1c2ccccc2c(-c2cccc3oc4cc(-c5c6ccccc6c(-c6ccccc6)c6ccccc56)ccc4c23)c2ccccc12 ; c1ccc(-c2c3ccccc3c(-c3cccc4oc5cc(-c6c7ccccc7c(-c7ccc8ccccc8c7)c7ccccc67)ccc5c34)c3ccccc23)cc1 ; c1ccc(-c2c3ccccc3c(-c3cccc4oc5cc(-c6c7ccccc7c(-c7cccc8ccccc78)c7ccccc67)ccc5c34)c3ccccc23)cc1 ; c1ccc(-c2c3ccccc3c(-c3cccc4oc5cc(-c6c7ccccc7c(-c7ccc(-c8cccc9ccccc89)cc7)c7ccccc67)ccc5c34)c3ccccc23)cc1]

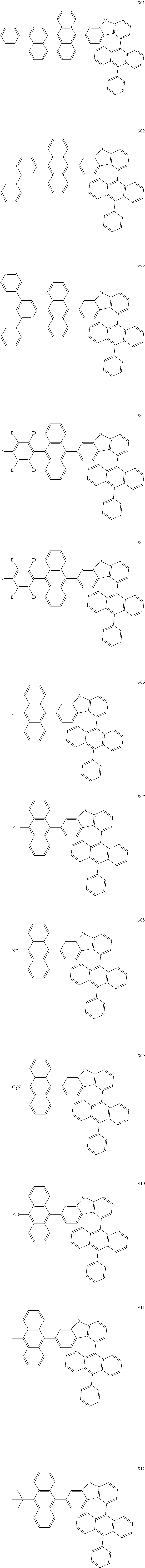

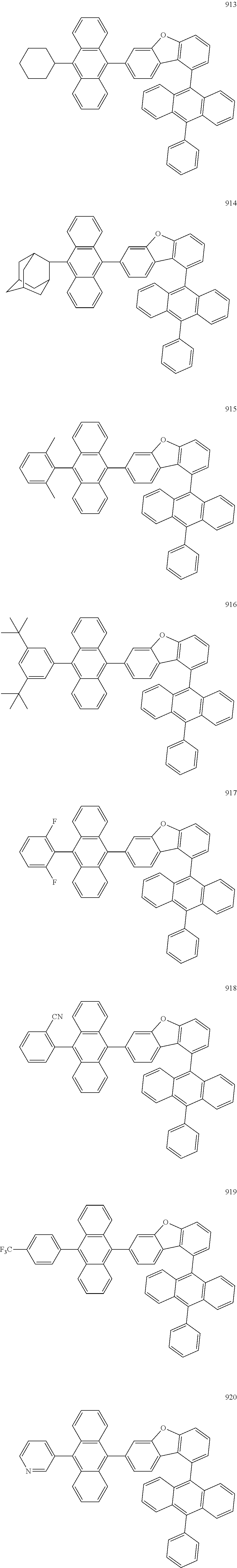

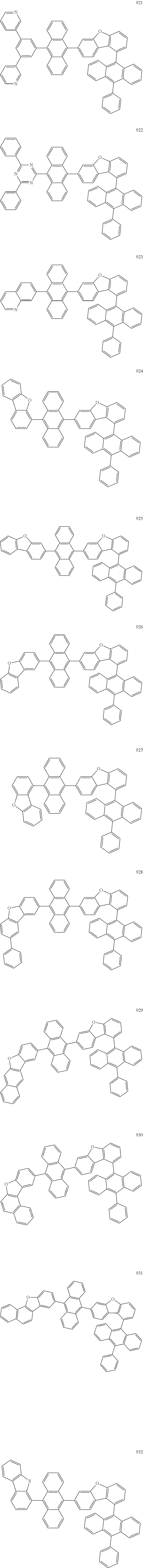

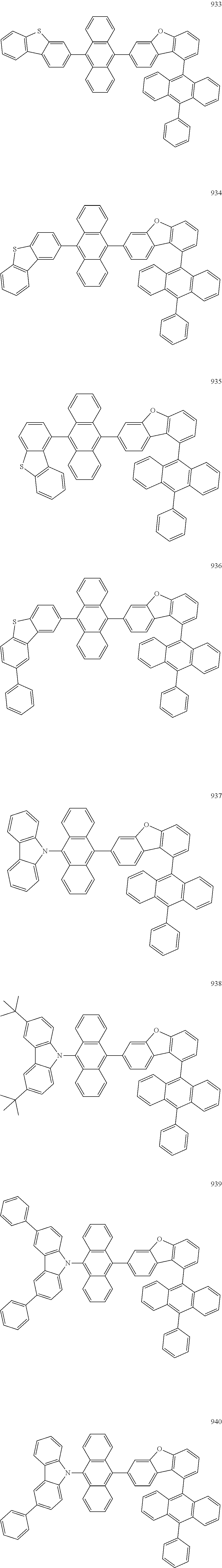

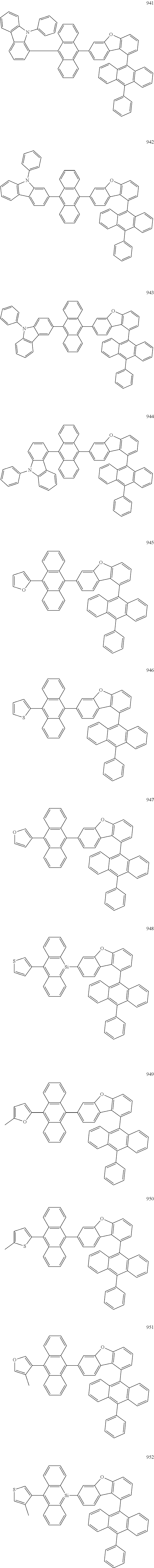

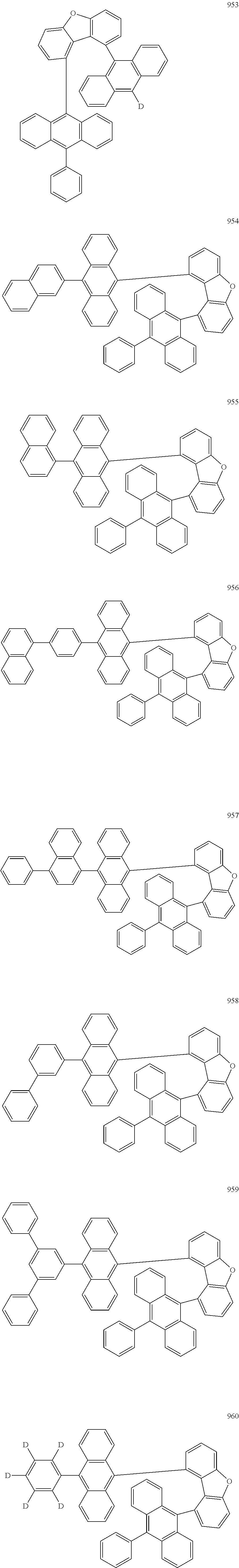

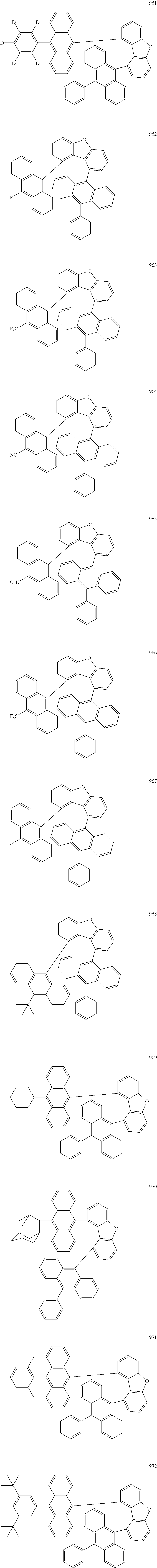

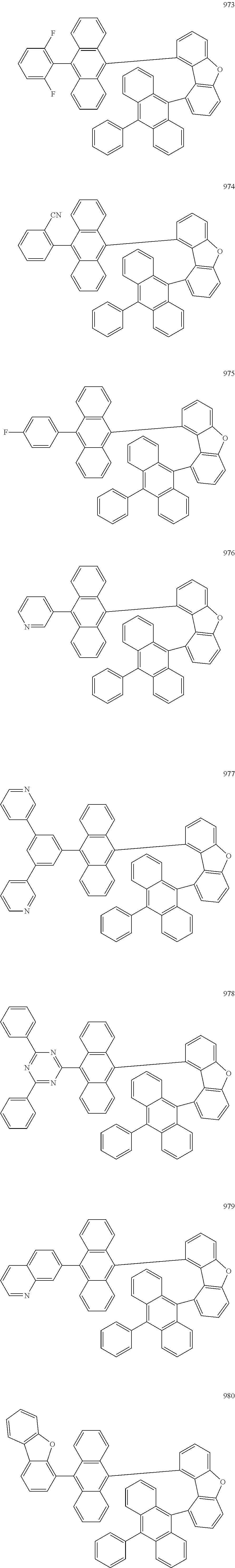

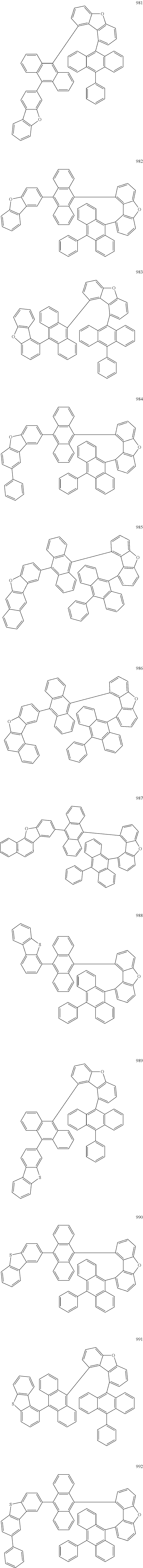

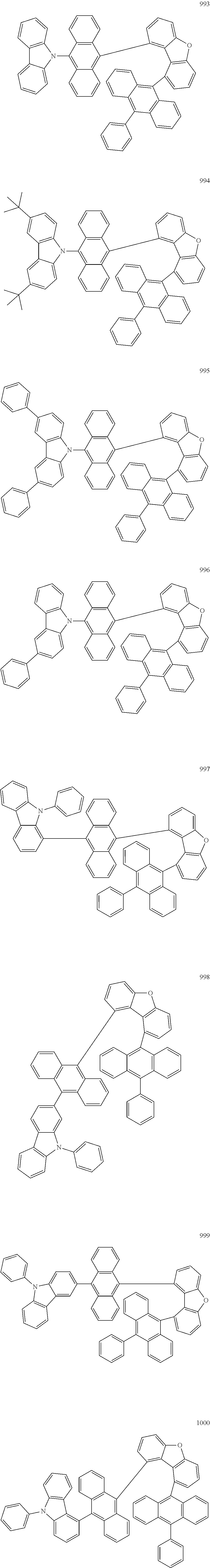

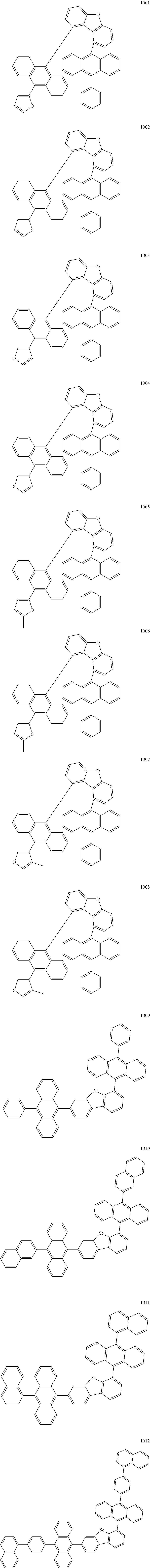

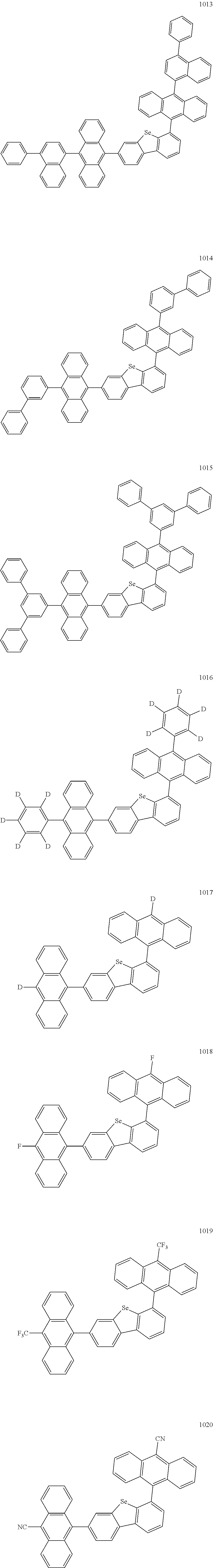

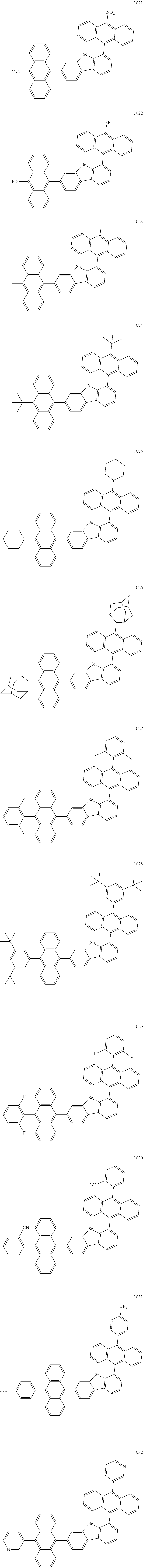

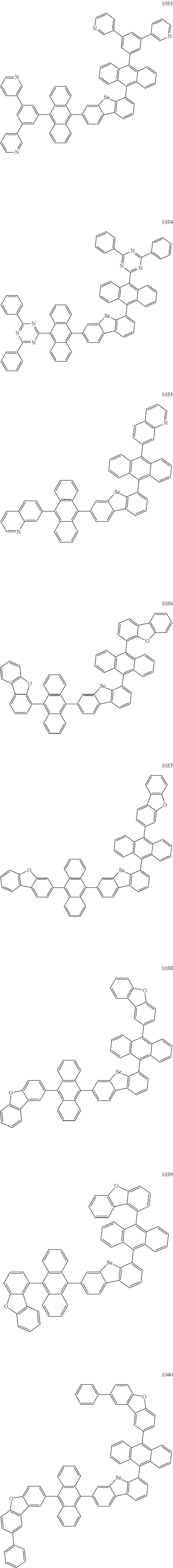

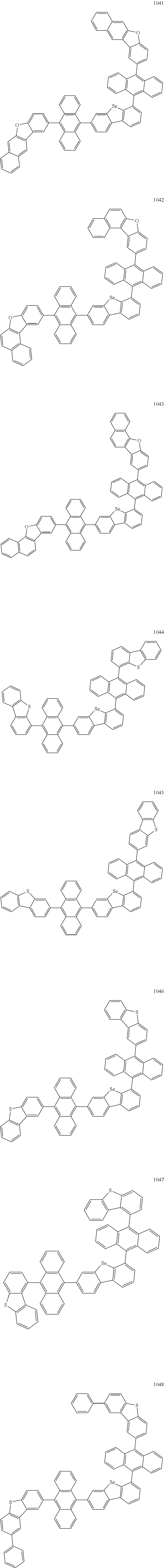

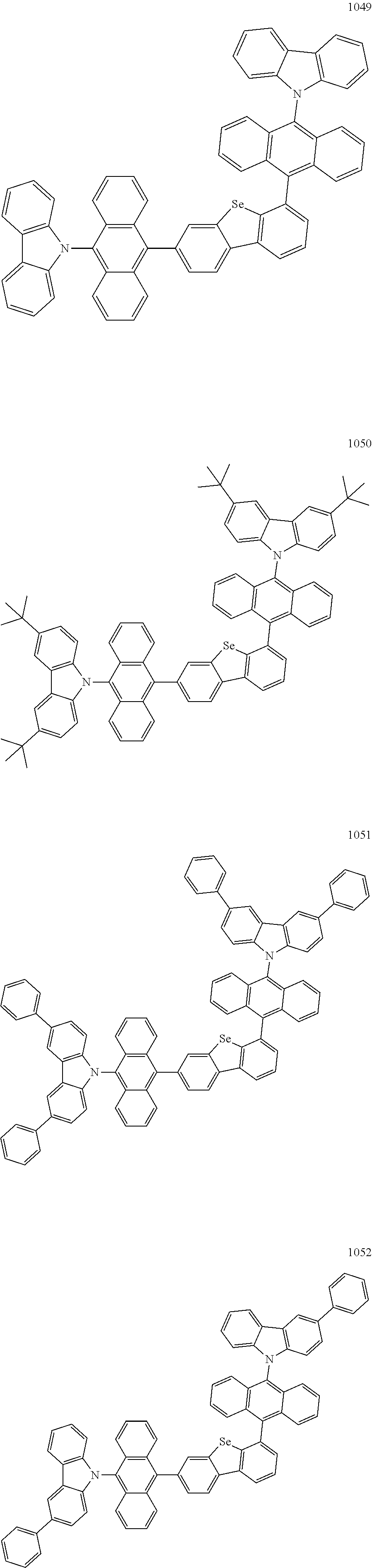

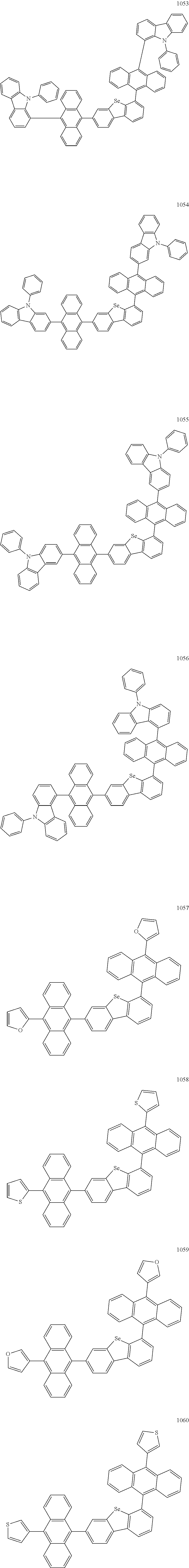

[chemical structure]

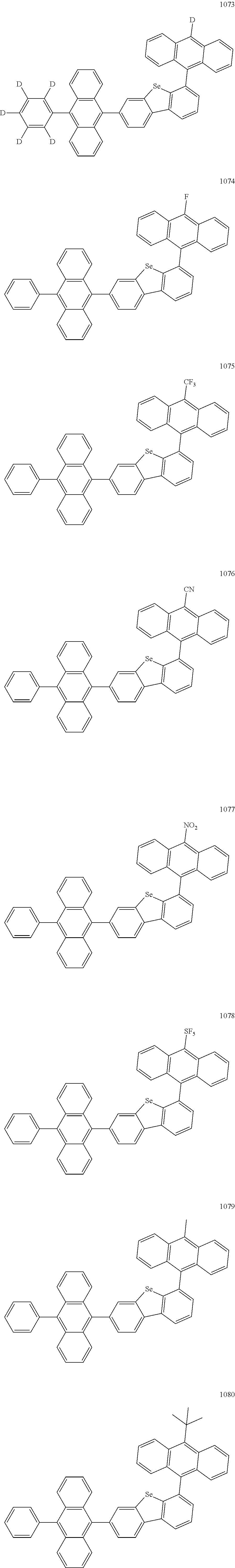

[chemical structure]

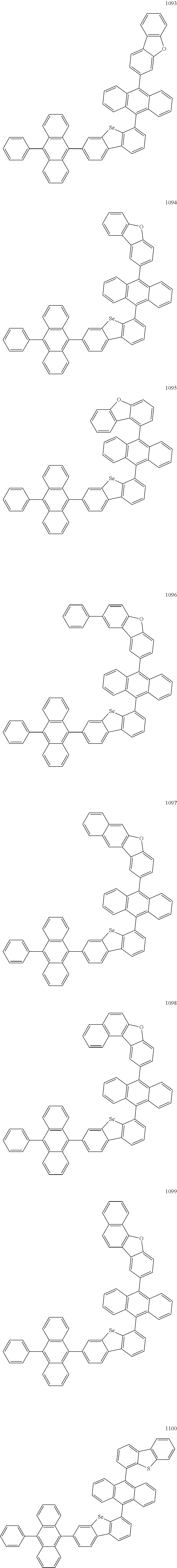

[chemical structure]

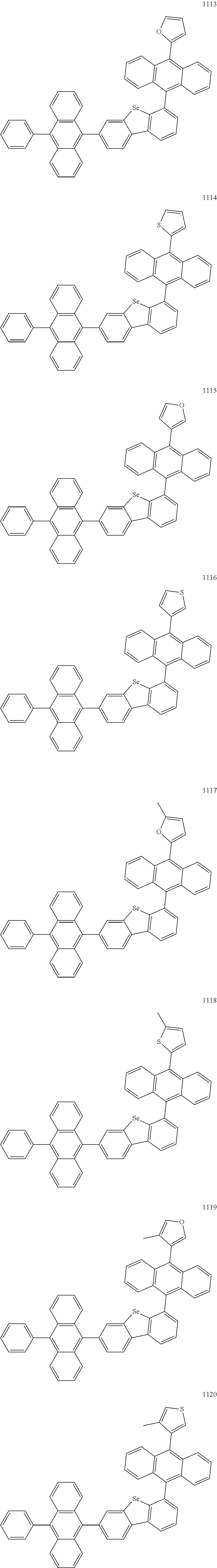

[chemical structure]

[chemical structure]

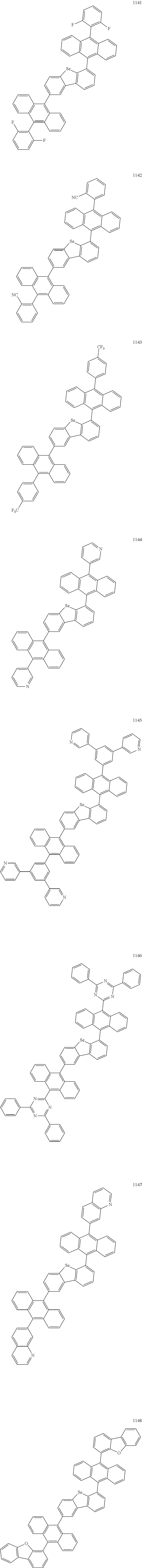

[chemical structure]

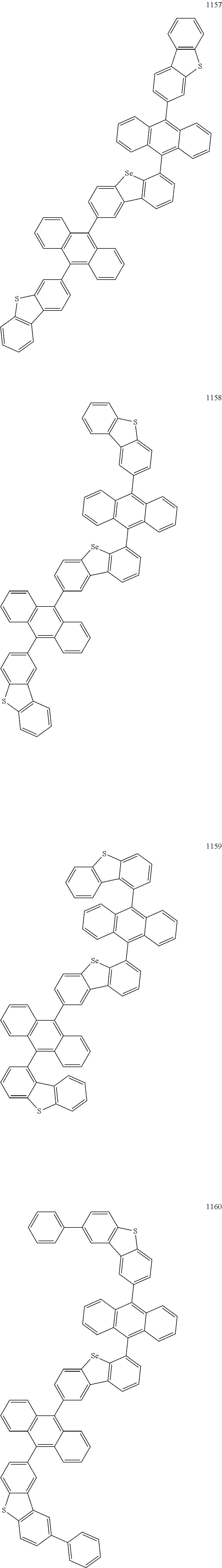

[chemical structure]

[chemical structure]

[chemical structure]

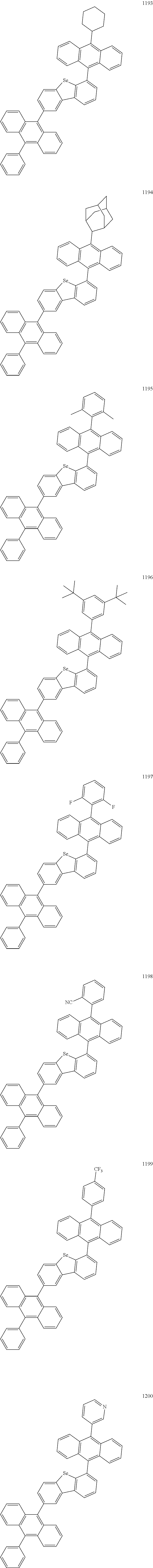

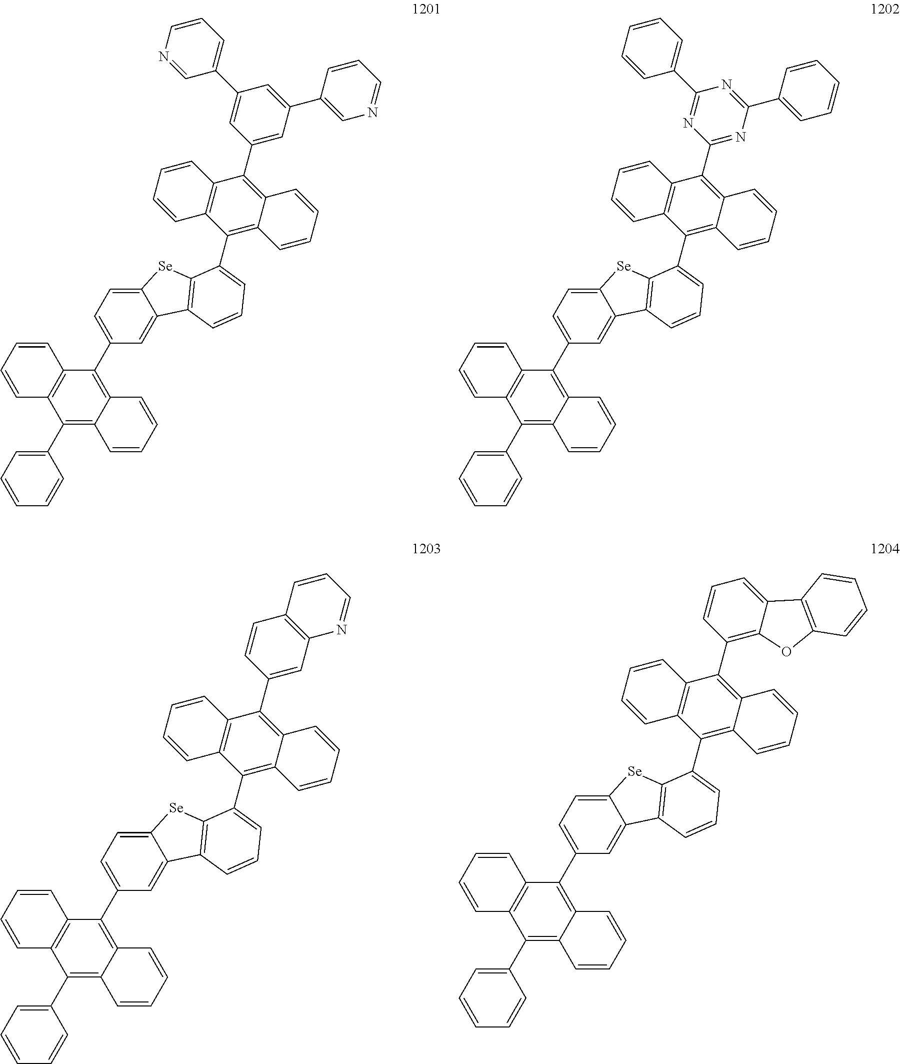 [SMILES: c1ccc(-c2c3ccccc3c(-c3ccc4[se]c5c(-c6c7ccccc7c(-c7cc(-c8cccnc8)cc(-c8cccnc8)c7)c7ccccc67)cccc5c4c3)c3ccccc23)cc1 ; c1ccc(-c2nc(-c3ccccc3)nc(-c3c4ccccc4c(-c4cccc5c4[se]c4ccc(-c6c7ccccc7c(-c7ccccc7)c7ccccc67)cc45)c4ccccc34)n2)cc1 ; c1ccc(-c2c3ccccc3c(-c3ccc4[se]c5c(-c6c7ccccc7c(-c7ccc8cccnc8c7)c7ccccc67)cccc5c4c3)c3ccccc23)cc1 ; c1ccc(-c2c3ccccc3c(-c3ccc4[se]c5c(-c6c7ccccc7c(-c7cccc8c7oc7ccccc78)c7ccccc67)cccc5c4c3)c3ccccc23)cc1]

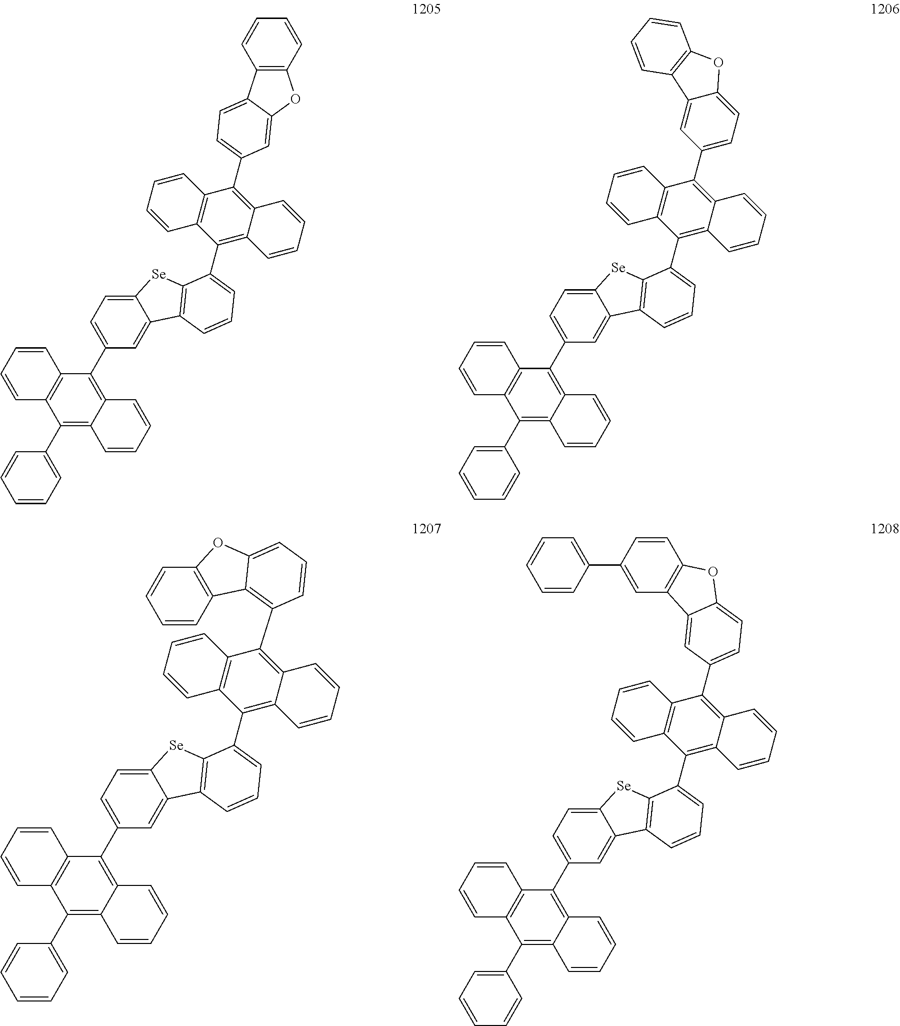 [SMILES: c1ccc(-c2c3ccccc3c(-c3ccc4[se]c5c(-c6c7ccccc7c(-c7ccc8c(c7)oc7ccccc78)c7ccccc67)cccc5c4c3)c3ccccc23)cc1 ; c1ccc(-c2c3ccccc3c(-c3ccc4[se]c5c(-c6c7ccccc7c(-c7ccc8oc9ccccc9c8c7)c7ccccc67)cccc5c4c3)c3ccccc23)cc1 ; c1ccc(-c2c3ccccc3c(-c3ccc4[se]c5c(-c6c7ccccc7c(-c7cccc8oc9ccccc9c78)c7ccccc67)cccc5c4c3)c3ccccc23)cc1 ; c1ccc(-c2ccc3oc4ccc(-c5c6ccccc6c(-c6cccc7c6[se]c6ccc(-c8c9ccccc9c(-c9ccccc9)c9ccccc89)cc67)c6ccccc56)cc4c3c2)cc1]

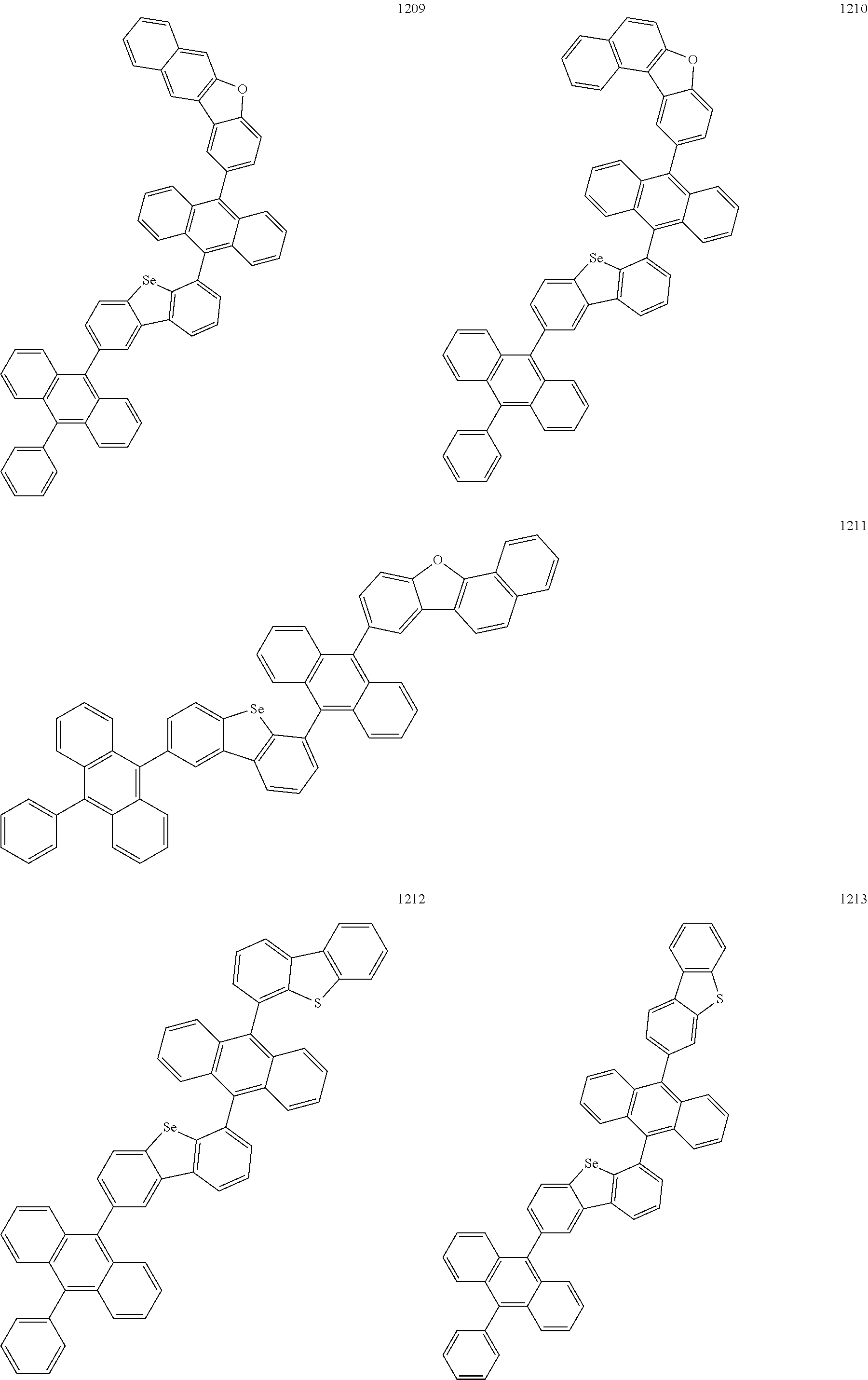

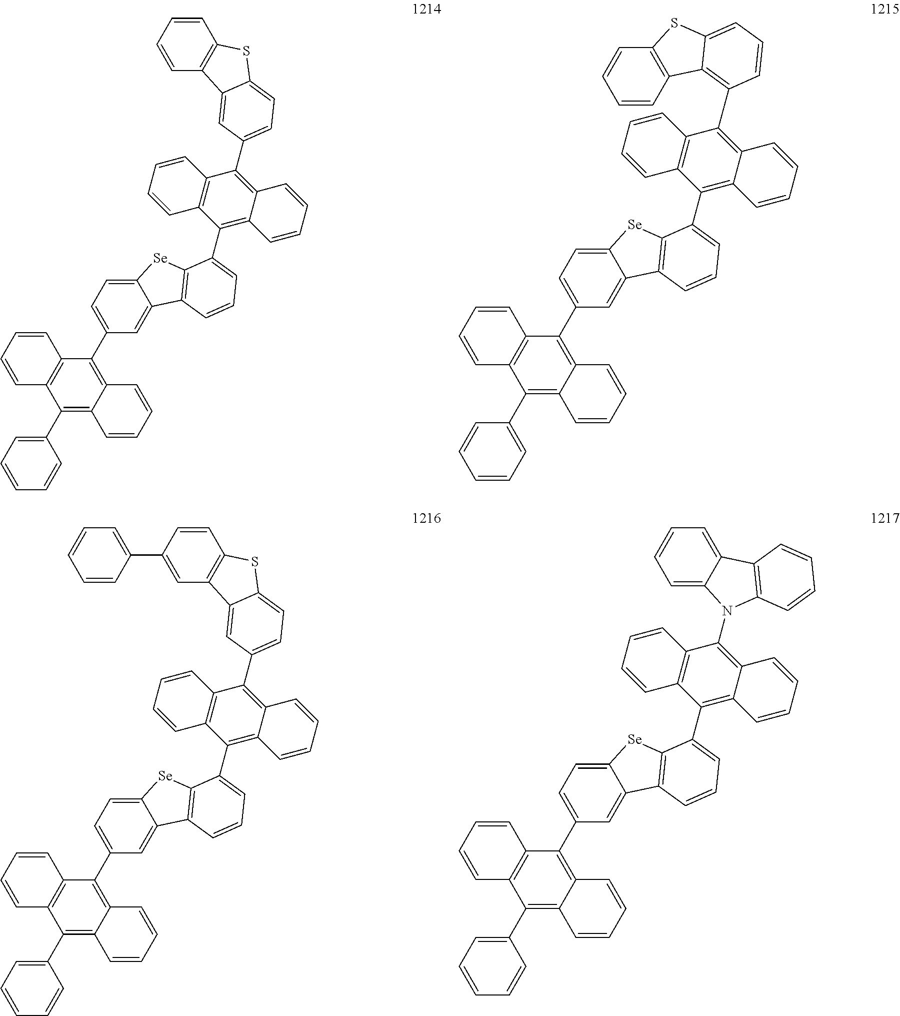 [SMILES: c1ccc(-c2c3ccccc3c(-c3ccc4[se]c5c(-c6c7ccccc7c(-c7ccc8sc9ccccc9c8c7)c7ccccc67)cccc5c4c3)c3ccccc23)cc1 ; c1ccc(-c2c3ccccc3c(-c3ccc4[se]c5c(-c6c7ccccc7c(-c7cccc8sc9ccccc9c78)c7ccccc67)cccc5c4c3)c3ccccc23)cc1 ; c1ccc(-c2ccc3sc4ccc(-c5c6ccccc6c(-c6cccc7c6[se]c6ccc(-c8c9ccccc9c(-c9ccccc9)c9ccccc89)cc67)c6ccccc56)cc4c3c2)cc1 ; c1ccc(-c2c3ccccc3c(-c3ccc4[se]c5c(-c6c7ccccc7c(-n7c8ccccc8c8ccccc87)c7ccccc67)cccc5c4c3)c3ccccc23)cc1]

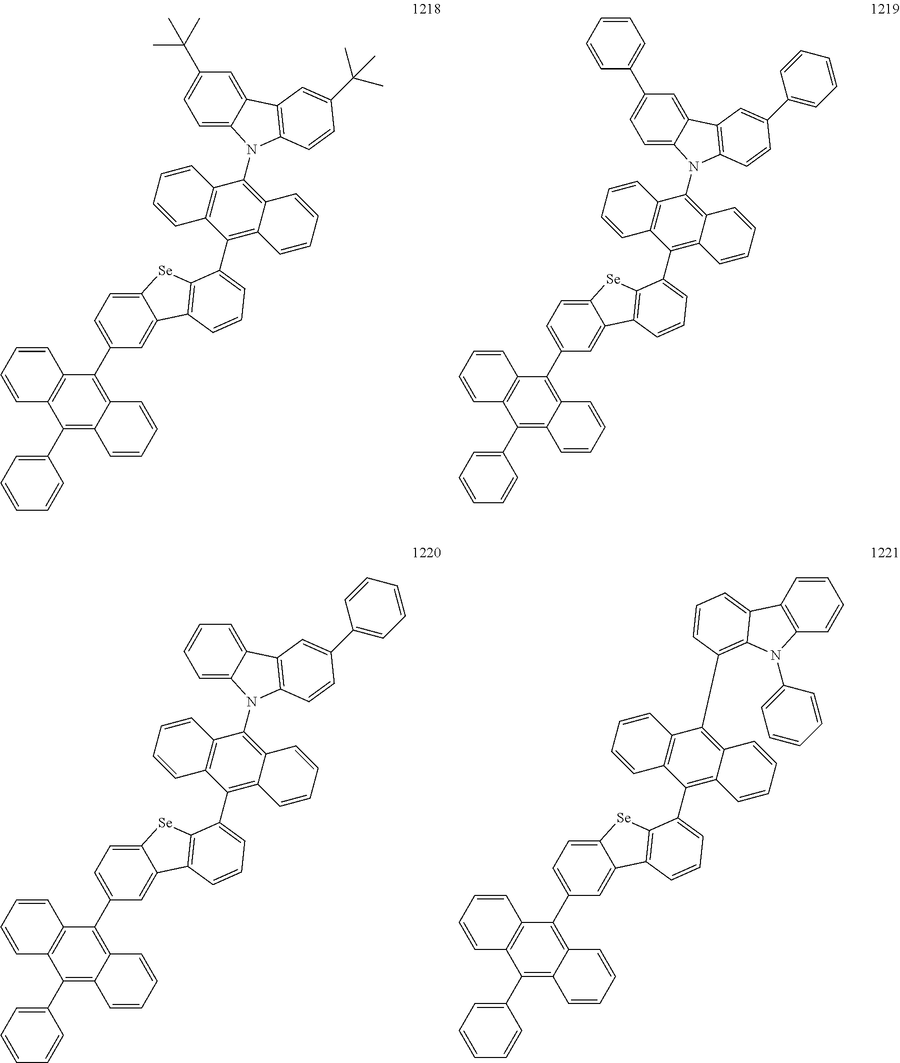 [SMILES: CC(C)(C)c1ccc2c(c1)c1cc(C(C)(C)C)ccc1n2-c1c2ccccc2c(-c2cccc3c2[se]c2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3cccc4c3[se]c3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3cccc4c3[se]c3ccc(-c5c6ccccc6c(-c6ccccc6)c6ccccc56)cc34)c3ccccc23)cc1 ; c1ccc(-c2c3ccccc3c(-c3ccc4[se]c5c(-c6c7ccccc7c(-c7cccc8c9ccccc9n(-c9ccccc9)c78)c7ccccc67)cccc5c4c3)c3ccccc23)cc1]

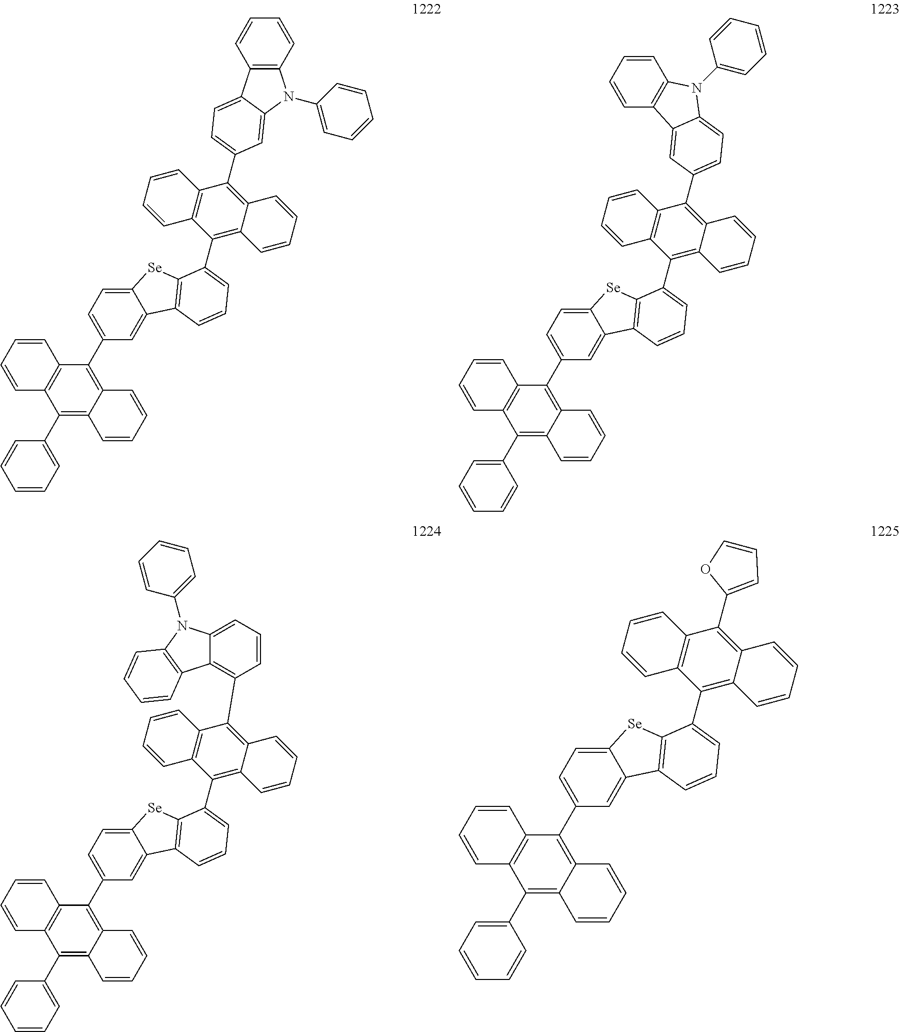 [SMILES: c1ccc(-c2c3ccccc3c(-c3ccc4[se]c5c(-c6c7ccccc7c(-c7ccc8c9ccccc9n(-c9ccccc9)c8c7)c7ccccc67)cccc5c4c3)c3ccccc23)cc1 ; c1ccc(-c2c3ccccc3c(-c3ccc4[se]c5c(-c6c7ccccc7c(-c7ccc8c(c7)c7ccccc7n8-c7ccccc7)c7ccccc67)cccc5c4c3)c3ccccc23)cc1 ; c1ccc(-c2c3ccccc3c(-c3ccc4[se]c5c(-c6c7ccccc7c(-c7cccc8c7c7ccccc7n8-c7ccccc7)c7ccccc67)cccc5c4c3)c3ccccc23)cc1 ; c1ccc(-c2c3ccccc3c(-c3ccc4[se]c5c(-c6c7ccccc7c(-c7ccco7)c7ccccc67)cccc5c4c3)c3ccccc23)cc1]

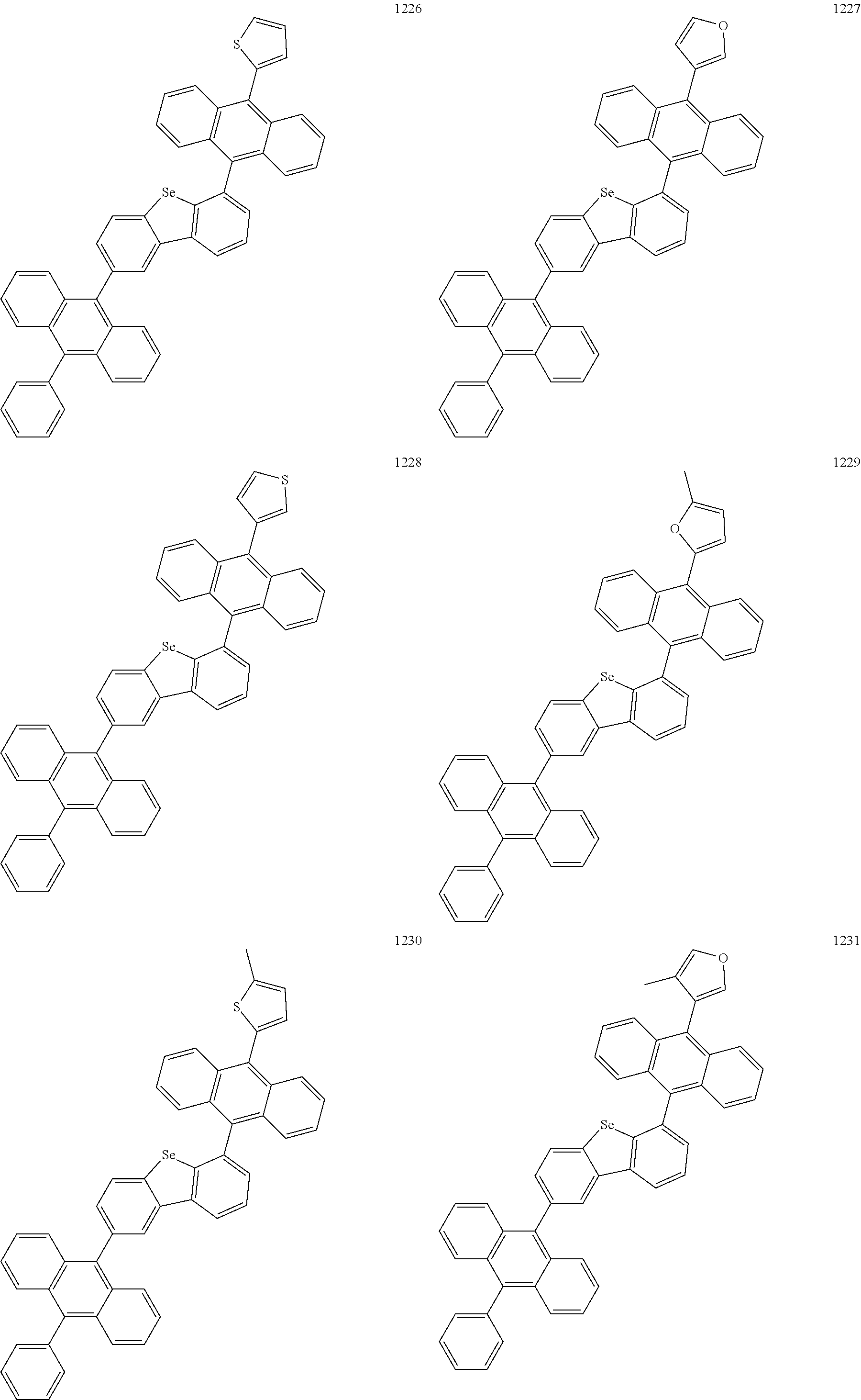

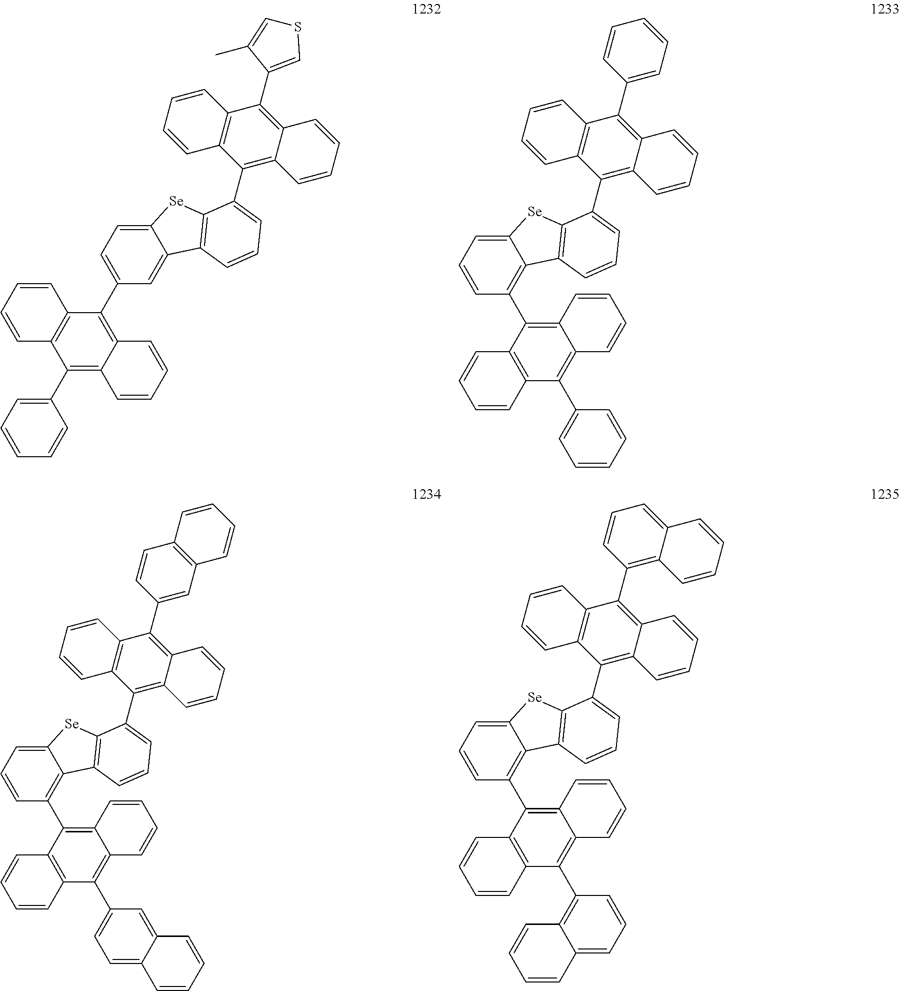

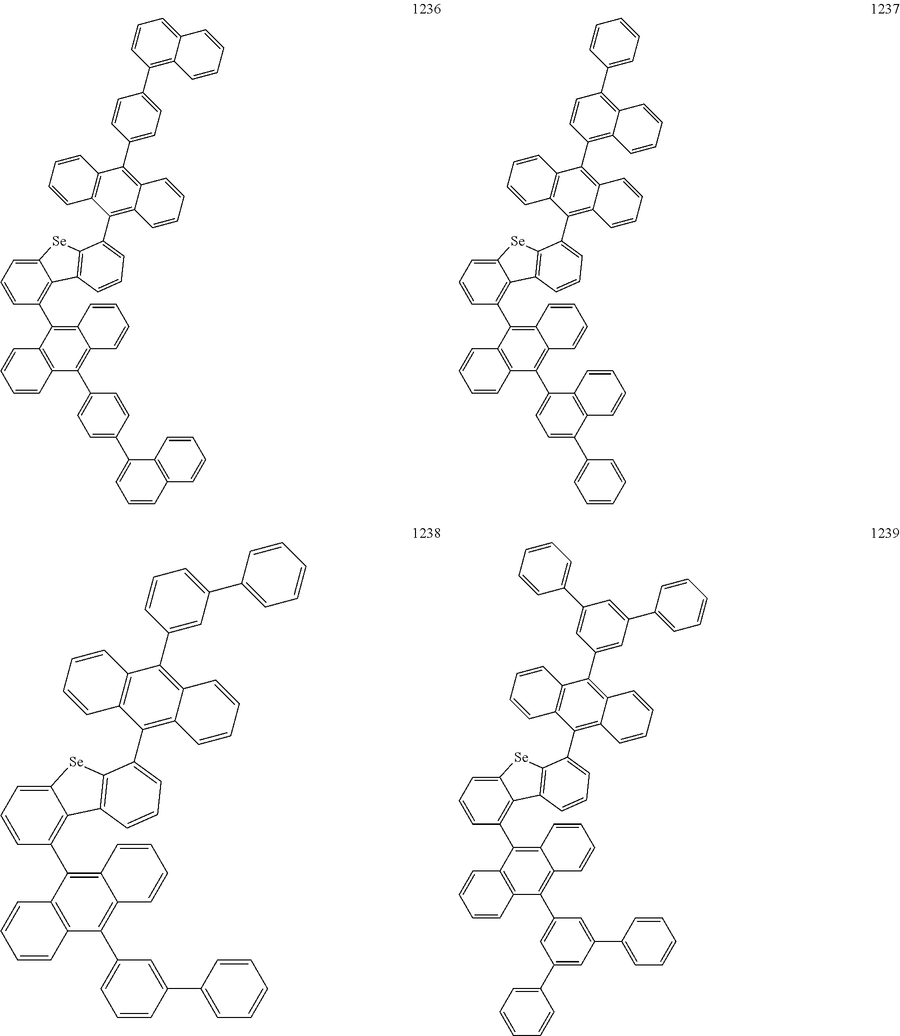

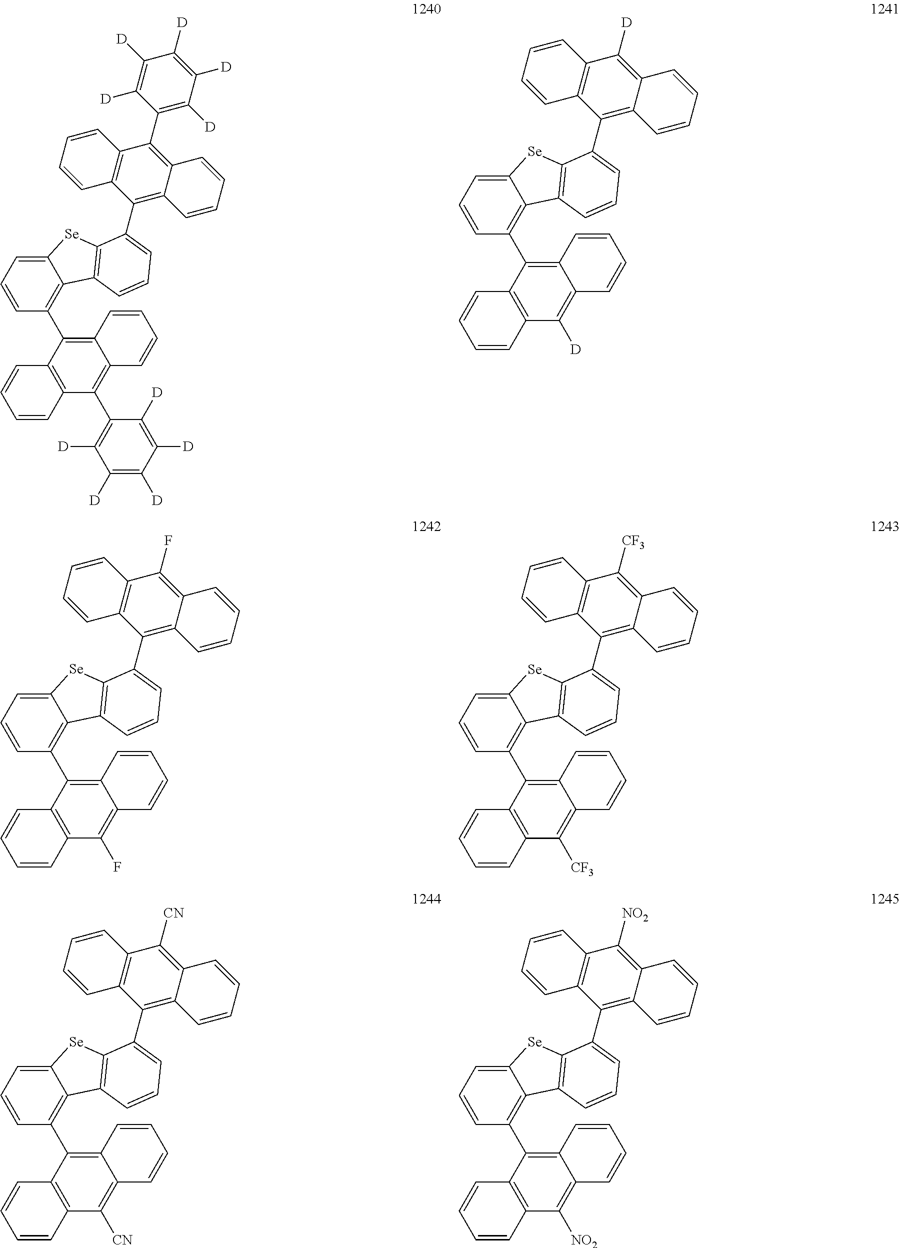

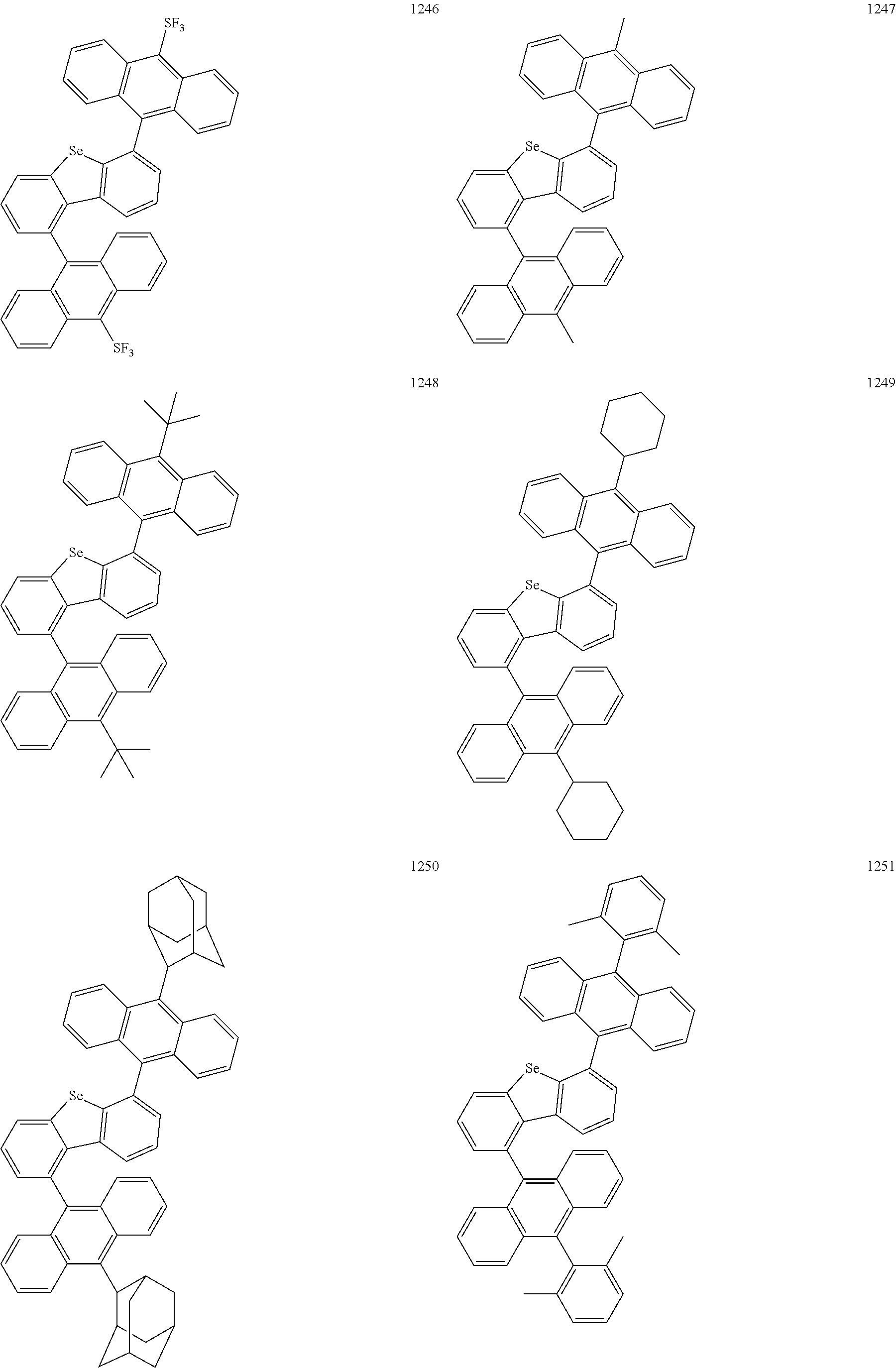

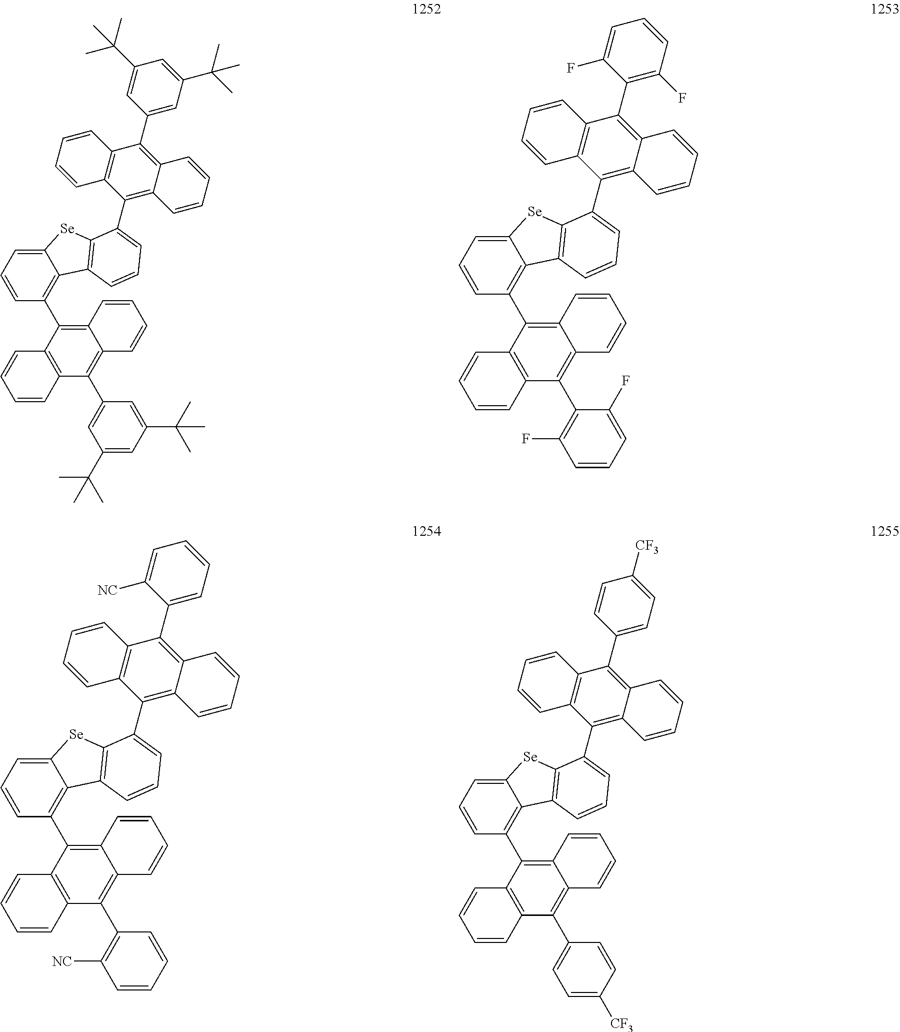 [SMILES: CC(C)(C)c1cc(-c2c3ccccc3c(-c3cccc4c3[se]c3cccc(-c5c6ccccc6c(-c6cc(C(C)(C)C)cc(C(C)(C)C)c6)c6ccccc56)c34)c3ccccc23)cc(C(C)(C)C)c1 ; Fc1cccc(F)c1-c1c2ccccc2c(-c2cccc3c2[se]c2cccc(-c4c5ccccc5c(-c5c(F)cccc5F)c5ccccc45)c23)c2ccccc12 ; N#Cc1ccccc1-c1c2ccccc2c(-c2cccc3c2[se]c2cccc(-c4c5ccccc5c(-c5ccccc5C#N)c5ccccc45)c23)c2ccccc12 ; FC(F)(F)c1ccc(-c2c3ccccc3c(-c3cccc4c3[se]c3cccc(-c5c6ccccc6c(-c6ccc(C(F)(F)F)cc6)c6ccccc56)c34)c3ccccc23)cc1]

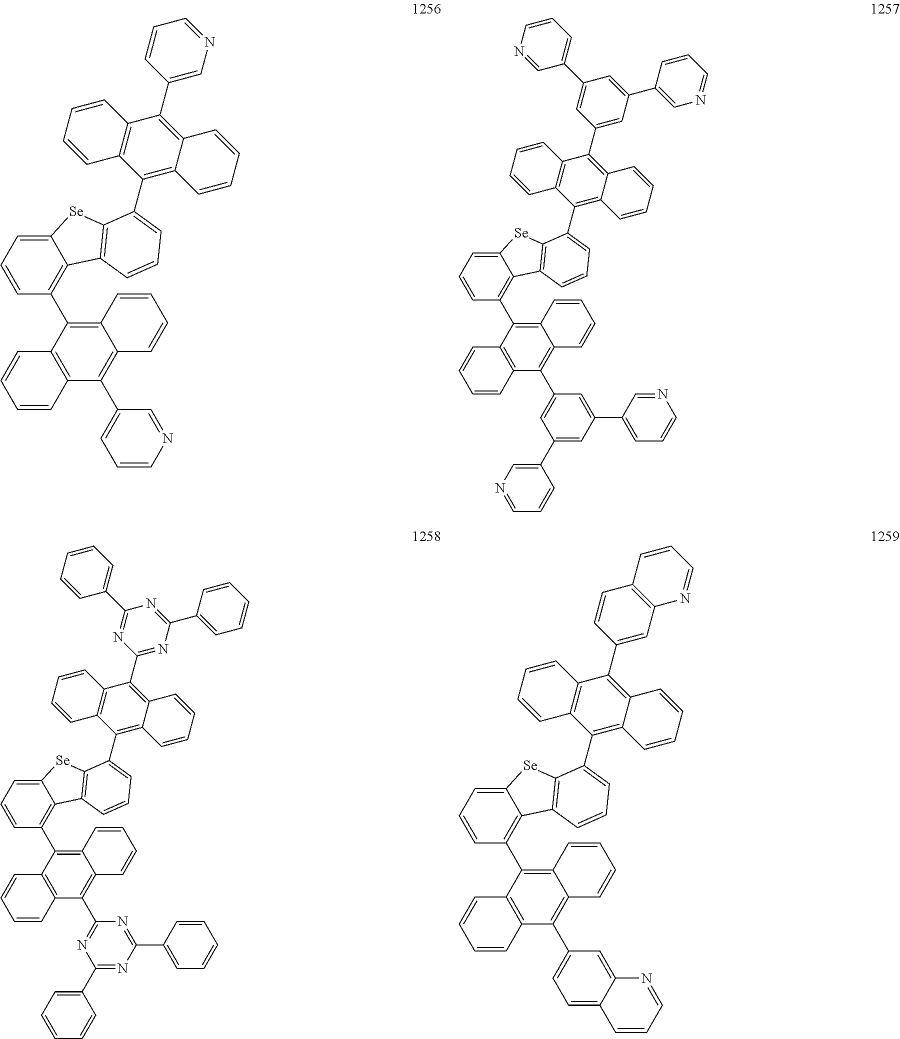 [SMILES: c1cncc(-c2c3ccccc3c(-c3cccc4c3[se]c3cccc(-c5c6ccccc6c(-c6cccnc6)c6ccccc56)c34)c3ccccc23)c1 ; c1cncc(-c2cc(-c3cccnc3)cc(-c3c4ccccc4c(-c4cccc5c4[se]c4cccc(-c6c7ccccc7c(-c7cc(-c8cccnc8)cc(-c8cccnc8)c7)c7ccccc67)c45)c4ccccc34)c2)c1 ; c1ccc(-c2nc(-c3ccccc3)nc(-c3c4ccccc4c(-c4cccc5c4[se]c4cccc(-c6c7ccccc7c(-c7nc(-c8ccccc8)nc(-c8ccccc8)n7)c7ccccc67)c45)c4ccccc34)n2)cc1 ; c1cnc2cc(-c3c4ccccc4c(-c4cccc5c4[se]c4cccc(-c6c7ccccc7c(-c7ccc8cccnc8c7)c7ccccc67)c45)c4ccccc34)ccc2c1]

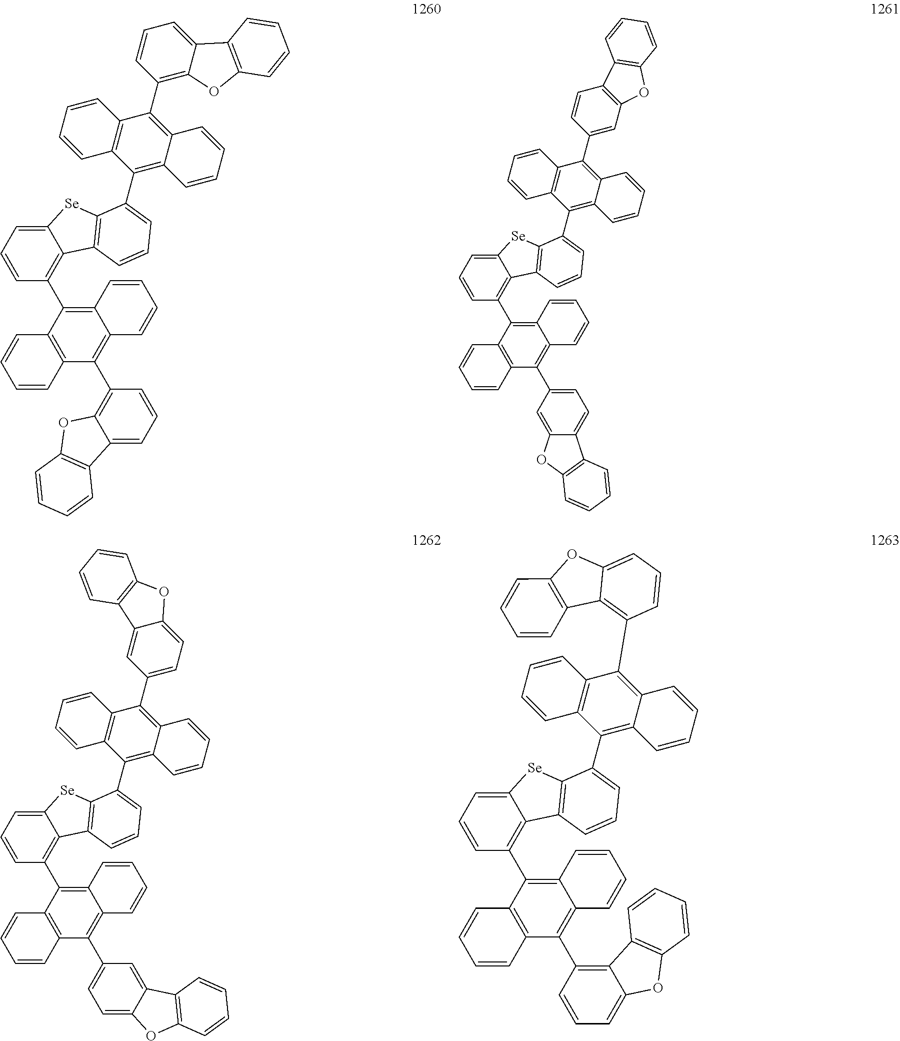 [SMILES: c1ccc2c(c1)oc1c(-c3c4ccccc4c(-c4cccc5c4[se]c4cccc(-c6c7ccccc7c(-c7cccc8c7oc7ccccc78)c7ccccc67)c45)c4ccccc34)cccc12 ; c1ccc2c(c1)oc1cc(-c3c4ccccc4c(-c4cccc5c4[se]c4cccc(-c6c7ccccc7c(-c7ccc8c(c7)oc7ccccc78)c7ccccc67)c45)c4ccccc34)ccc12 ; c1ccc2c(c1)oc1ccc(-c3c4ccccc4c(-c4cccc5c4[se]c4cccc(-c6c7ccccc7c(-c7ccc8oc9ccccc9c8c7)c7ccccc67)c45)c4ccccc34)cc12 ; c1ccc2c(c1)oc1cccc(-c3c4ccccc4c(-c4cccc5c4[se]c4cccc(-c6c7ccccc7c(-c7cccc8oc9ccccc9c78)c7ccccc67)c45)c4ccccc34)c12]

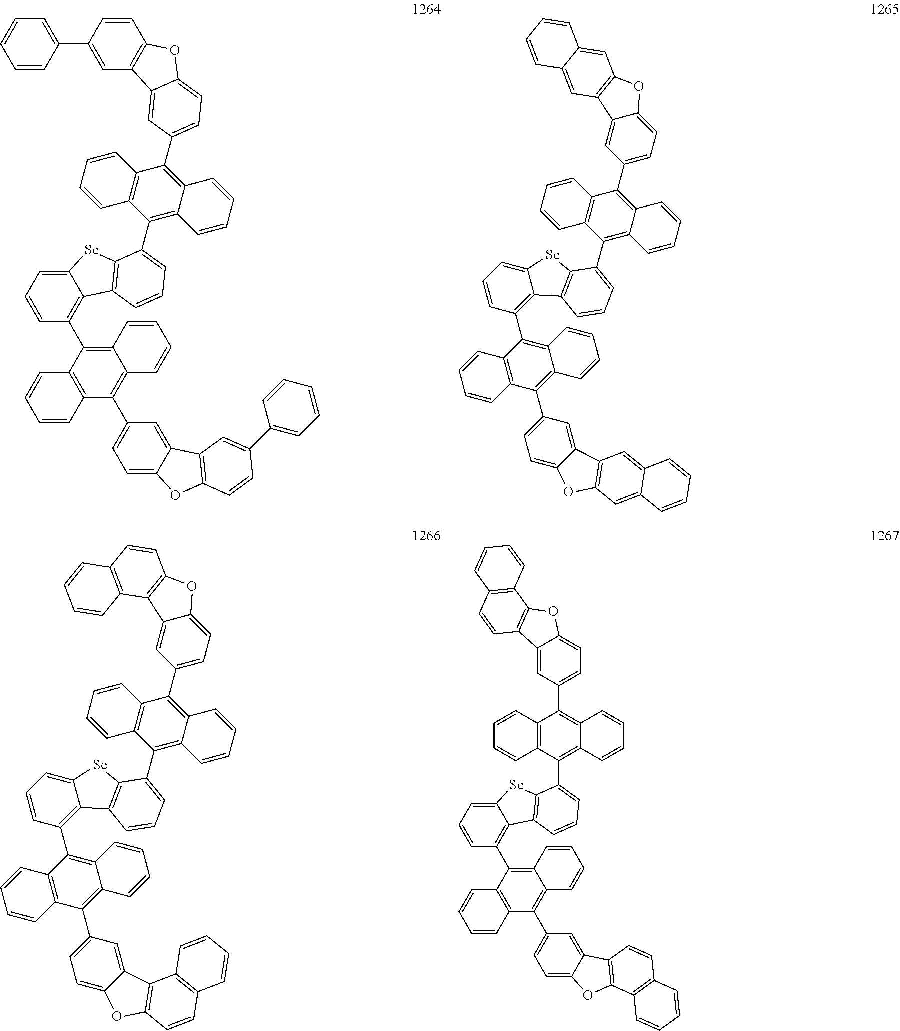

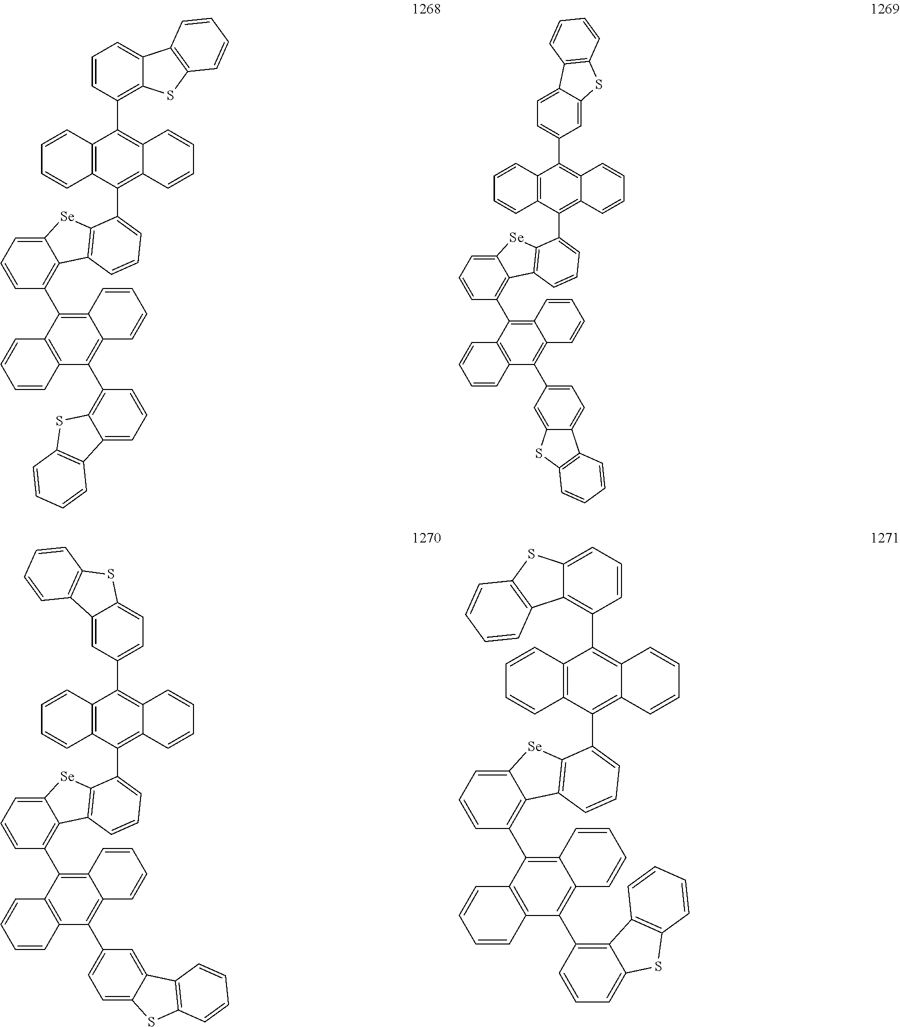

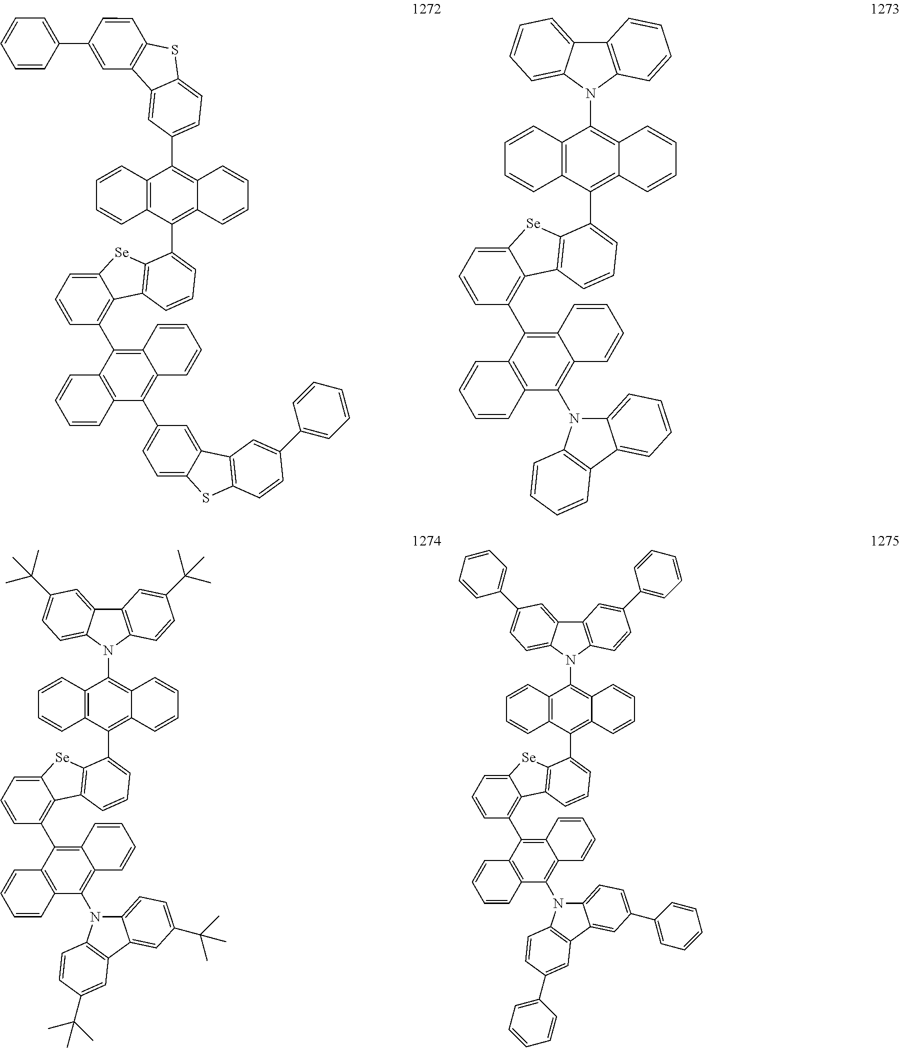

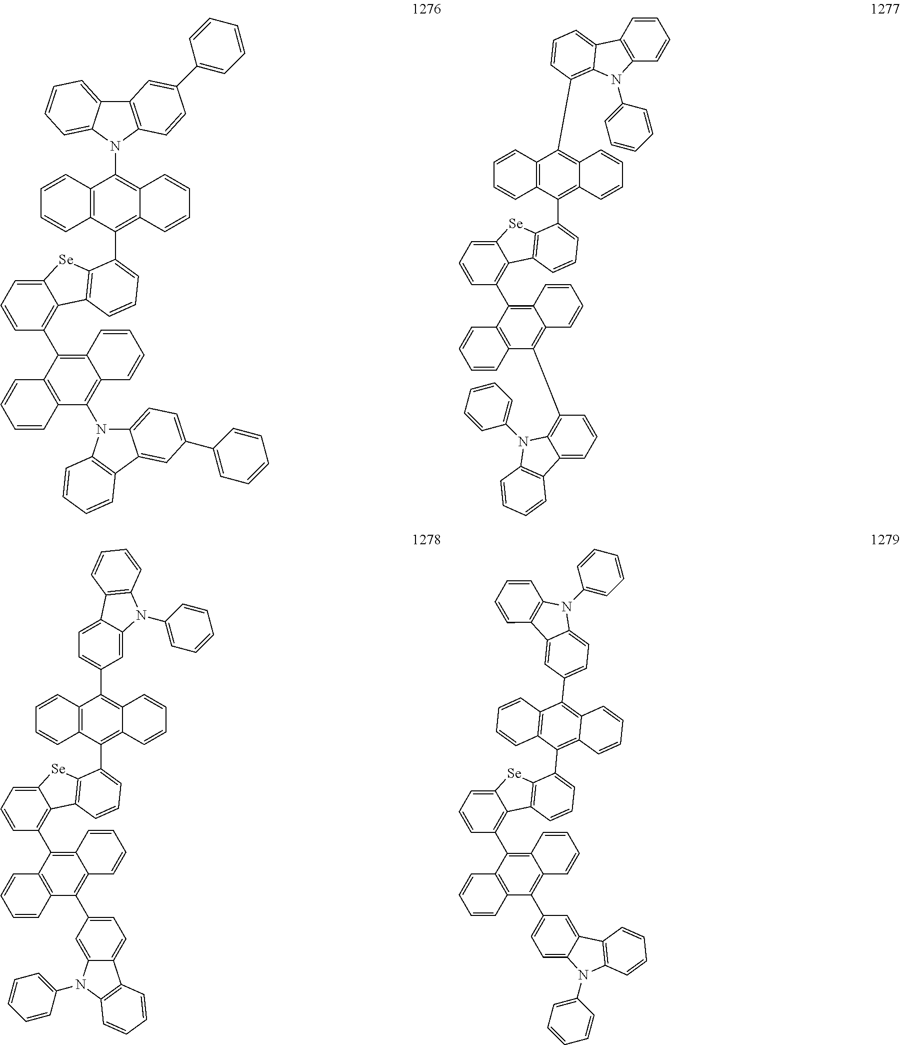

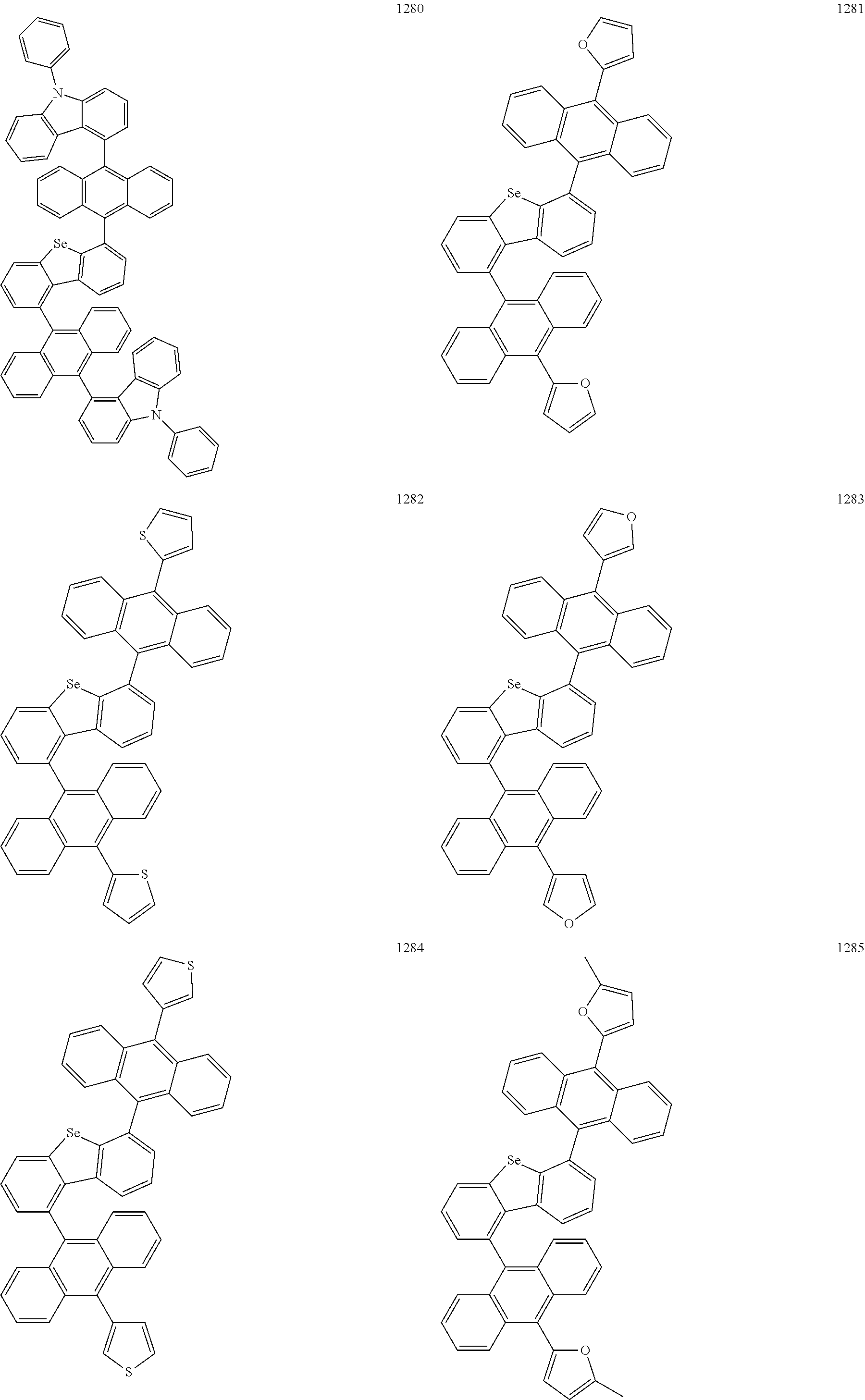

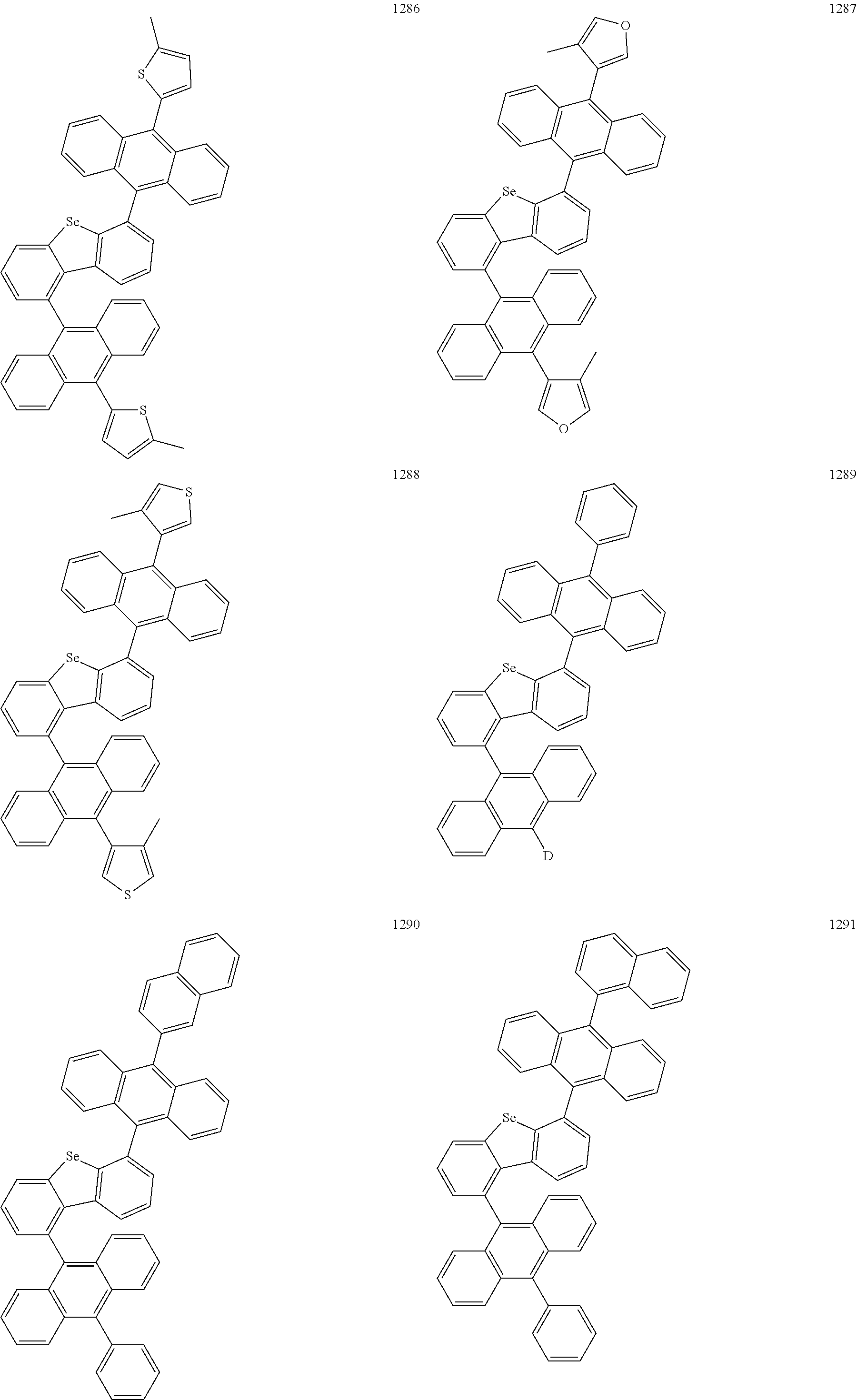

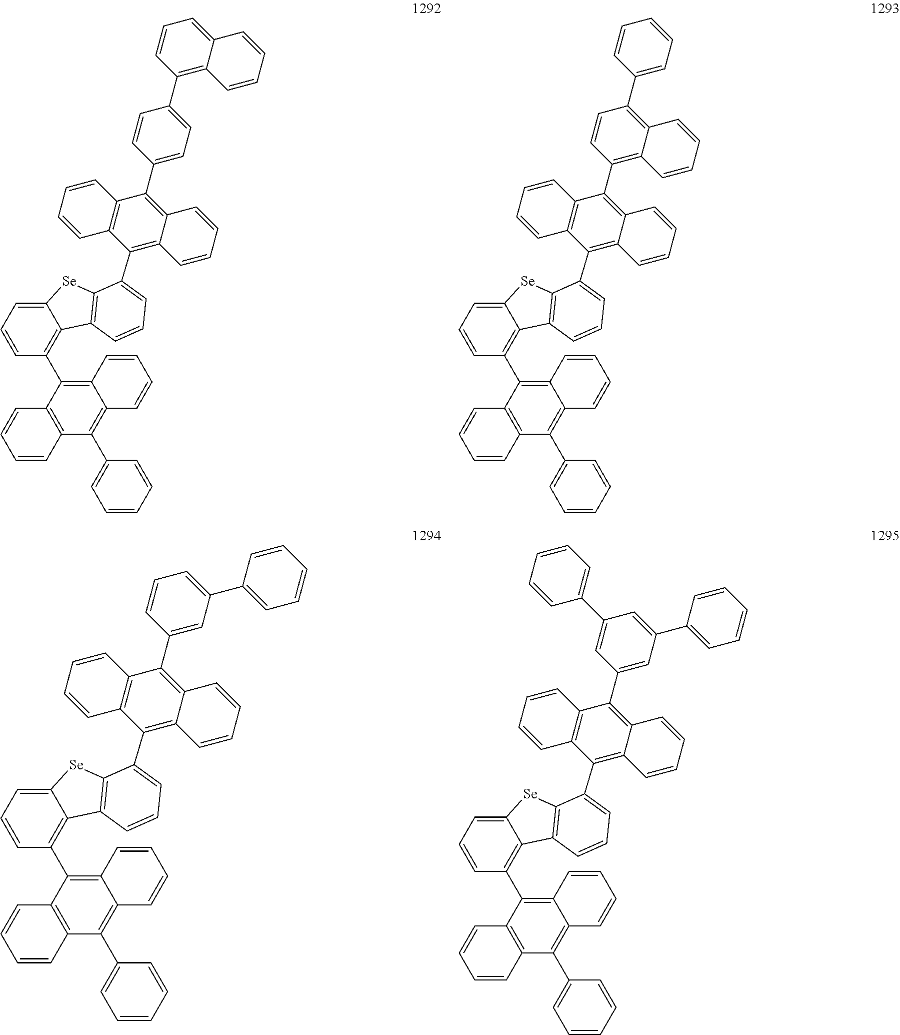

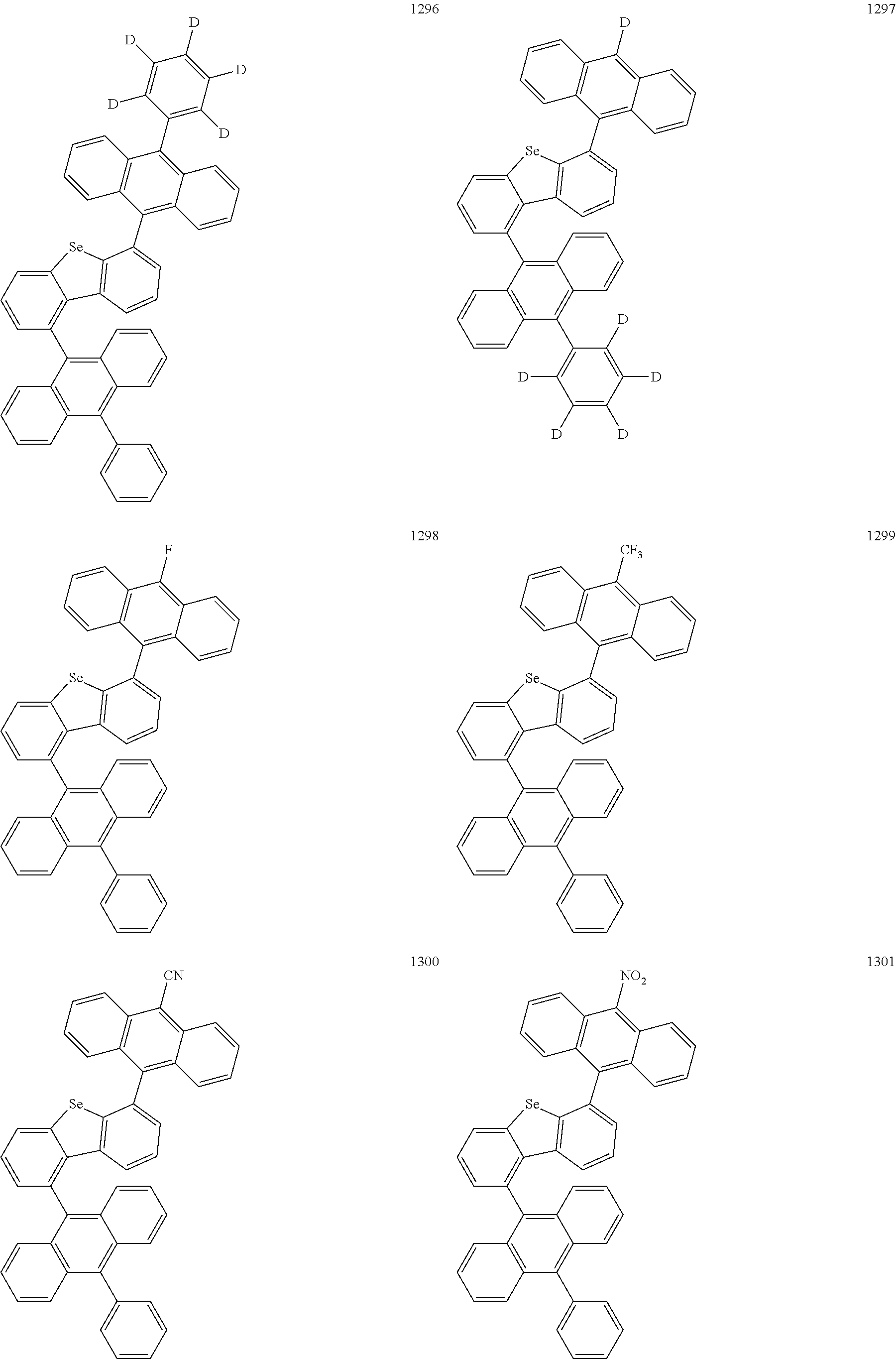

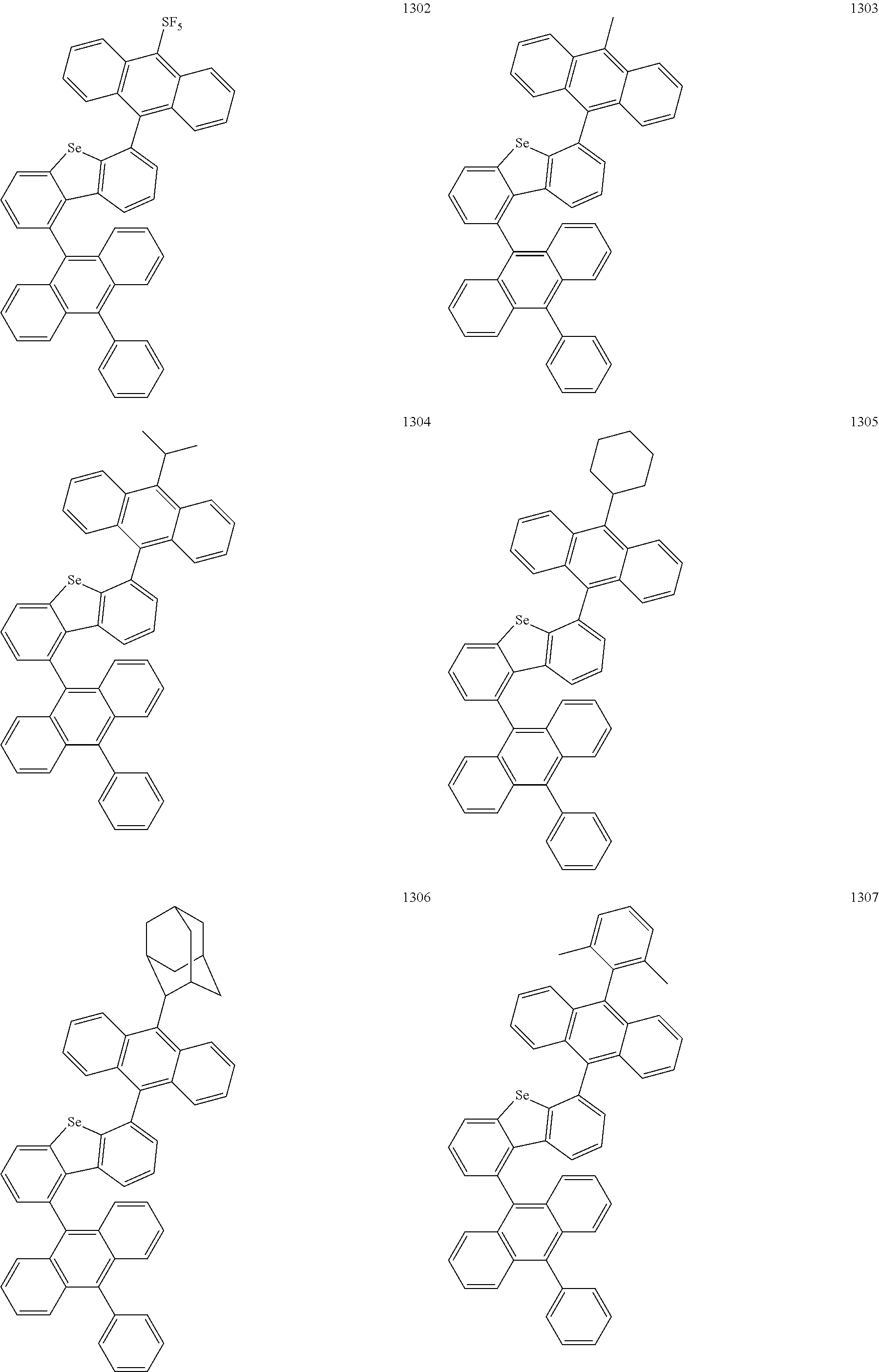

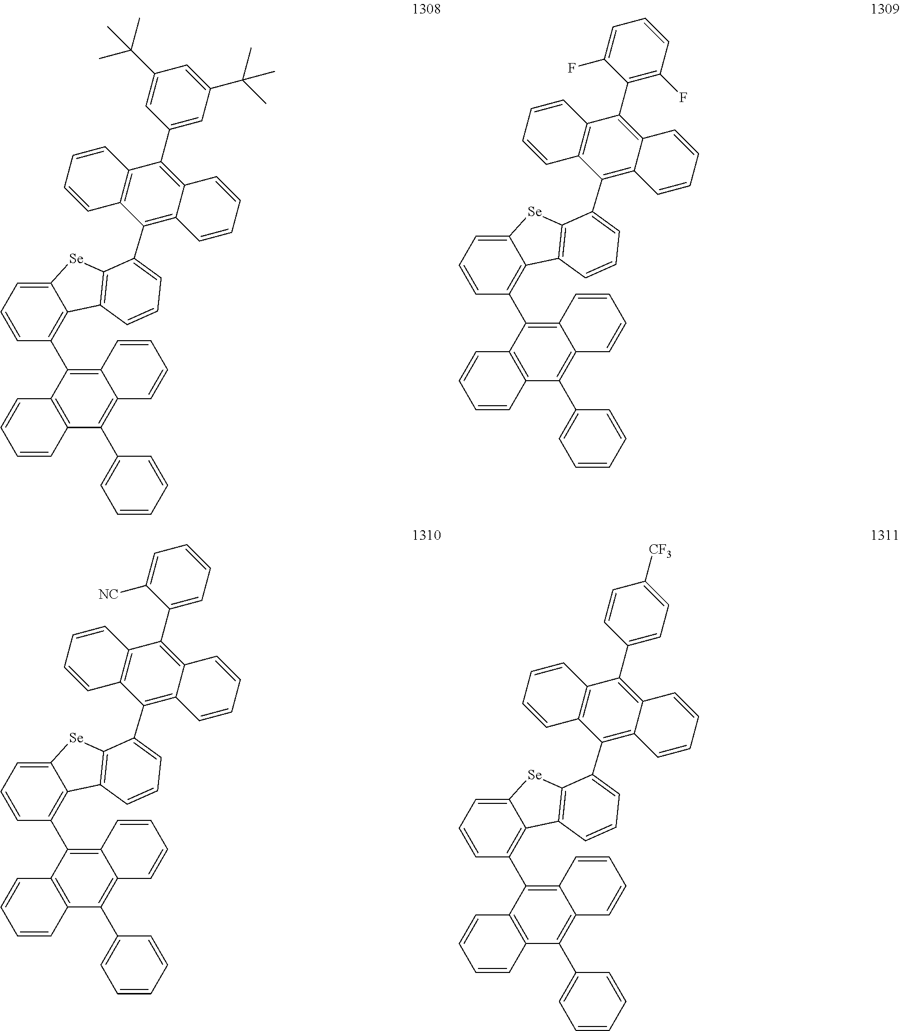

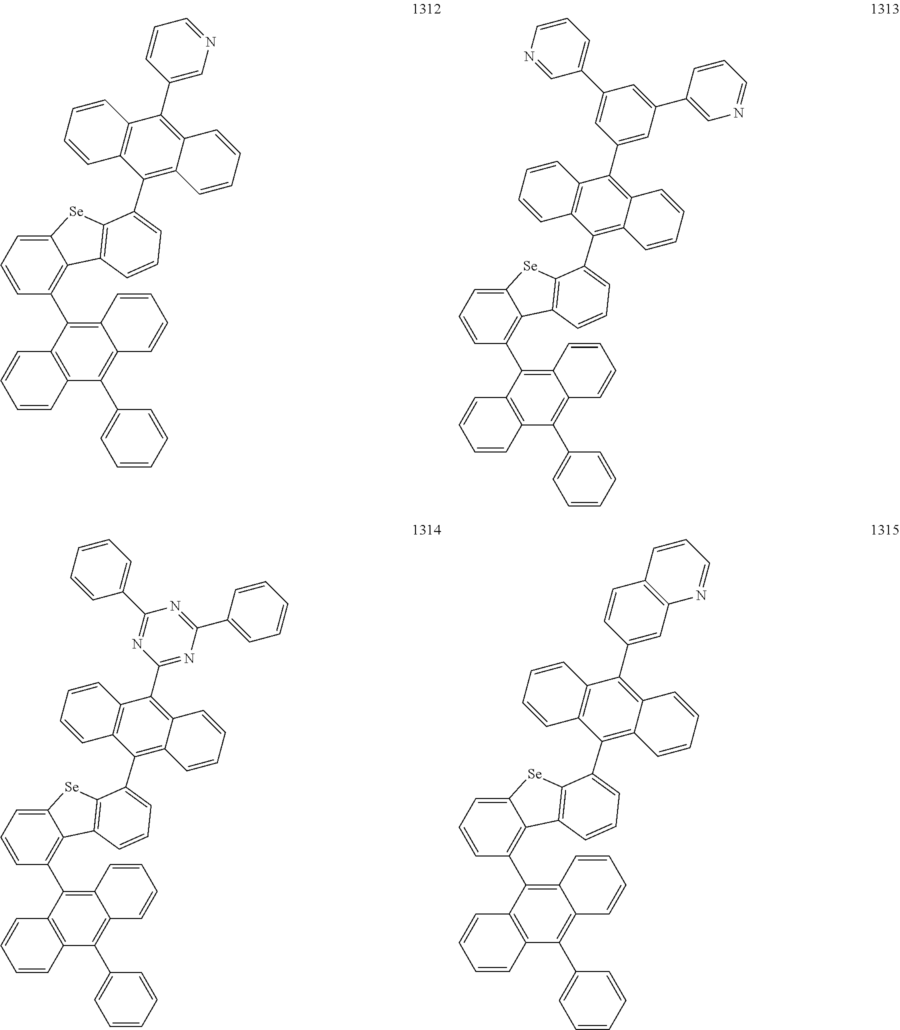 [SMILES: c1ccc(-c2c3ccccc3c(-c3cccc4[se]c5c(-c6c7ccccc7c(-c7cccnc7)c7ccccc67)cccc5c34)c3ccccc23)cc1 ; c1ccc(-c2c3ccccc3c(-c3cccc4[se]c5c(-c6c7ccccc7c(-c7cc(-c8cccnc8)cc(-c8cccnc8)c7)c7ccccc67)cccc5c34)c3ccccc23)cc1 ; c1ccc(-c2nc(-c3ccccc3)nc(-c3c4ccccc4c(-c4cccc5c4[se]c4cccc(-c6c7ccccc7c(-c7ccccc7)c7ccccc67)c45)c4ccccc34)n2)cc1 ; c1ccc(-c2c3ccccc3c(-c3cccc4[se]c5c(-c6c7ccccc7c(-c7ccc8cccnc8c7)c7ccccc67)cccc5c34)c3ccccc23)cc1]

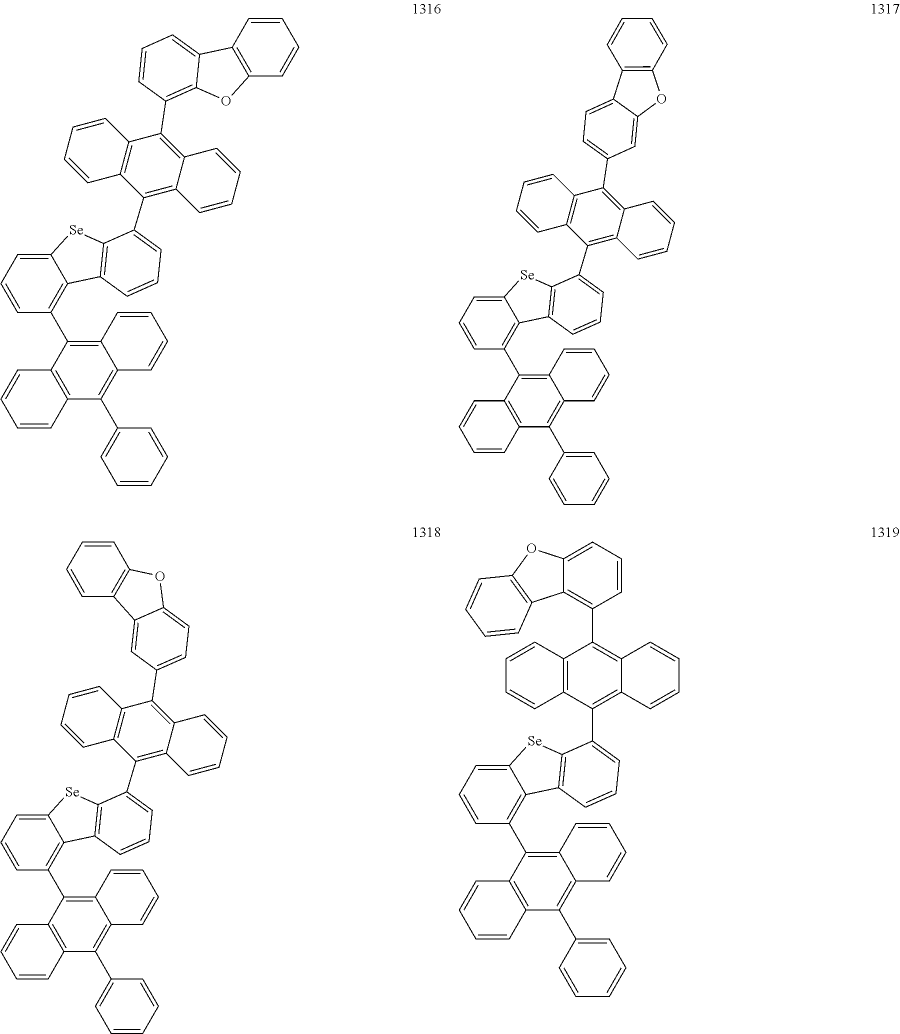

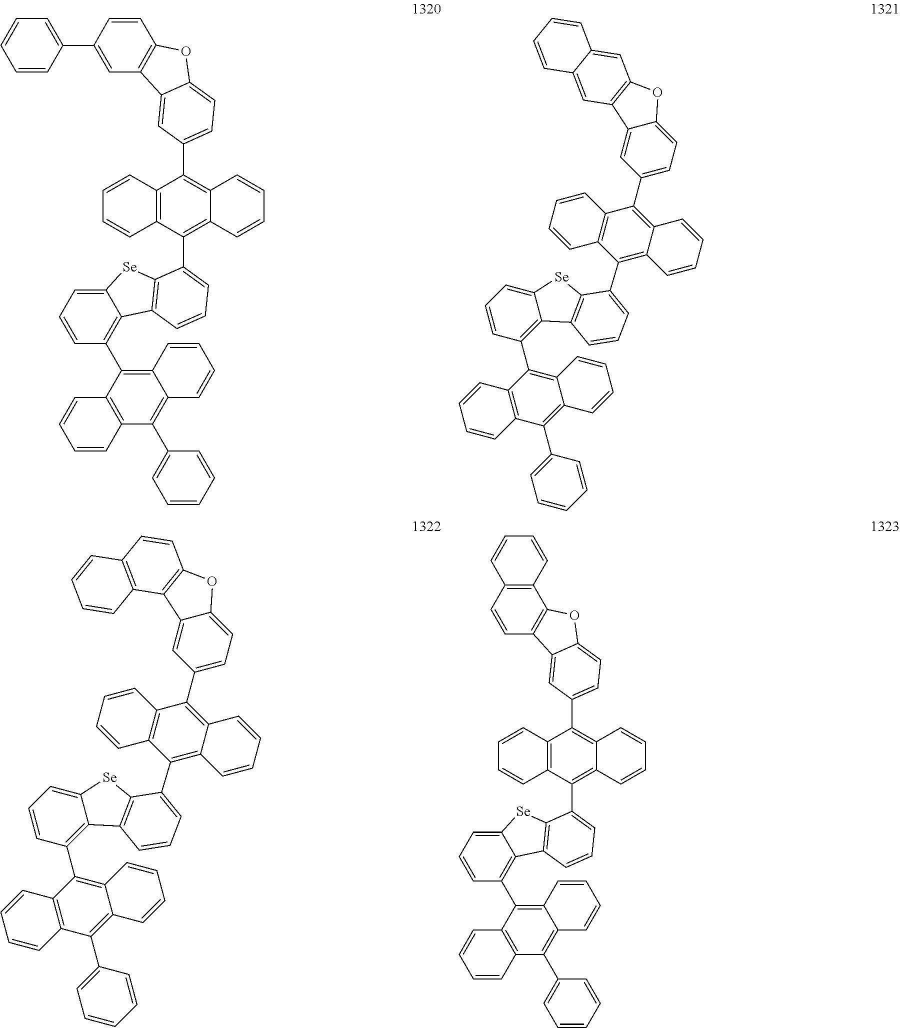

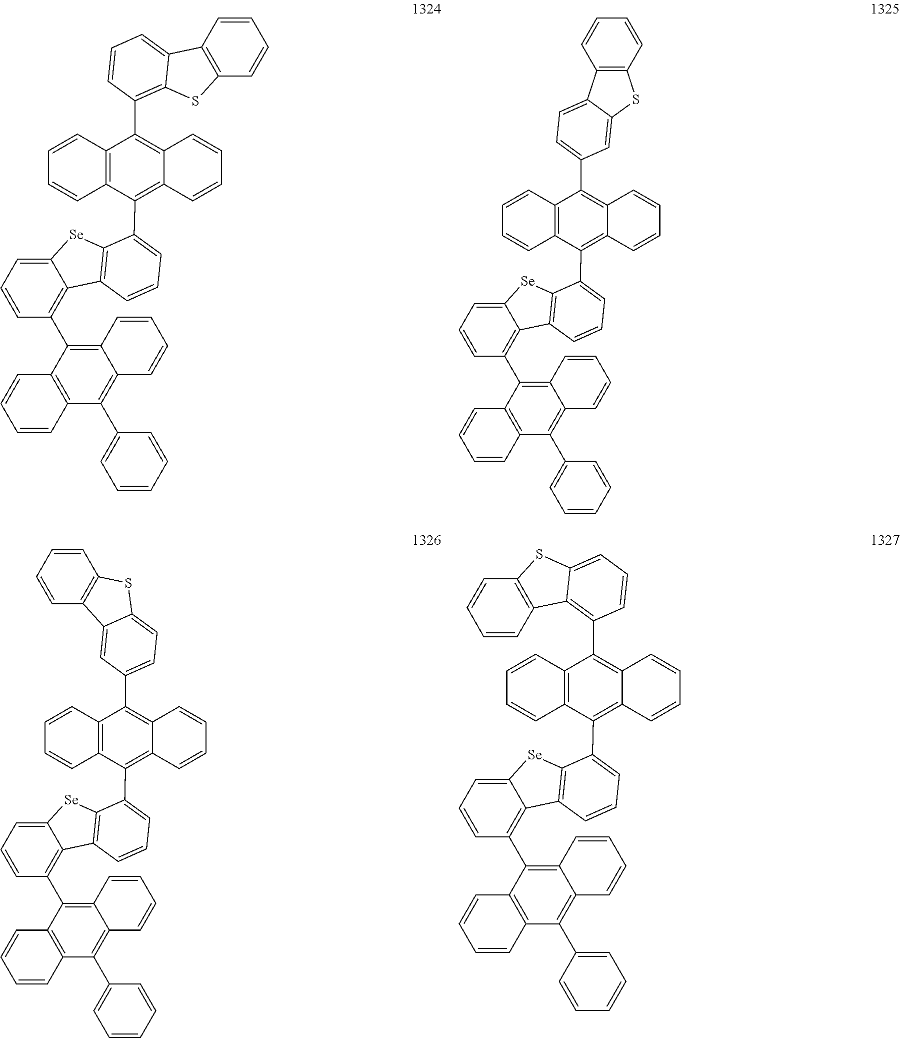

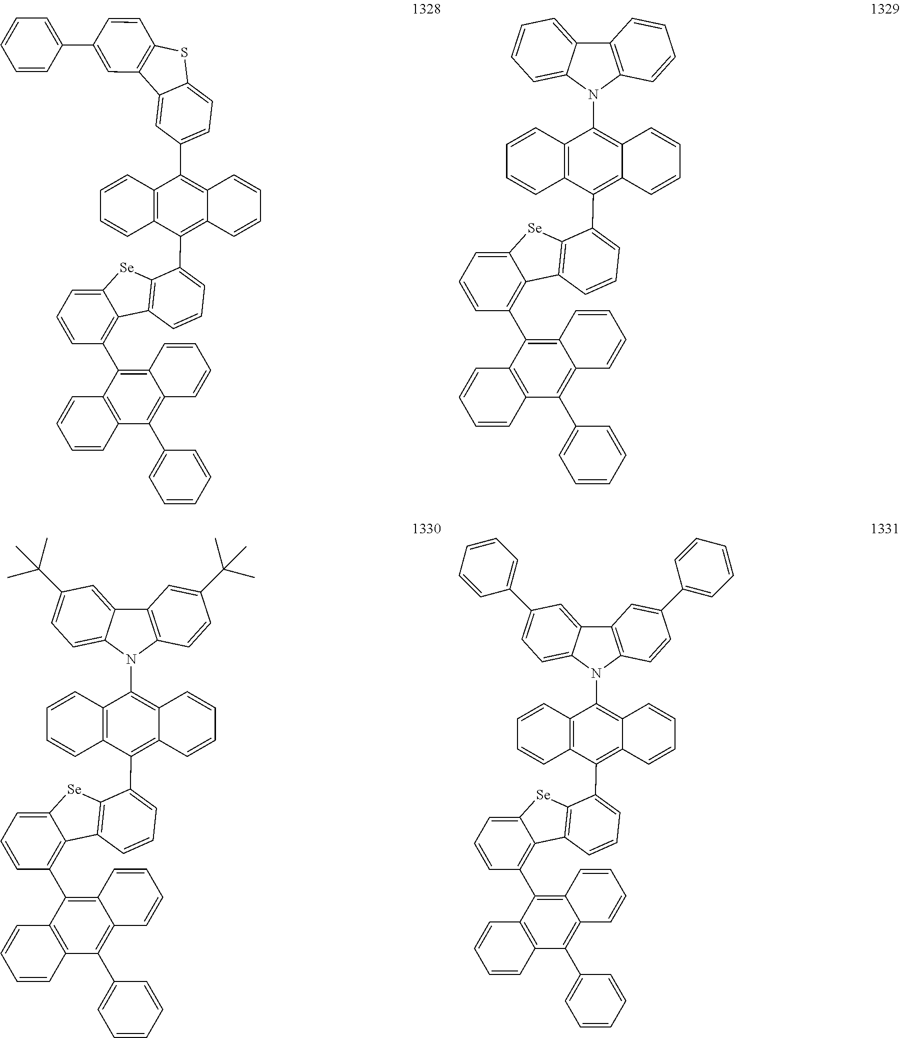

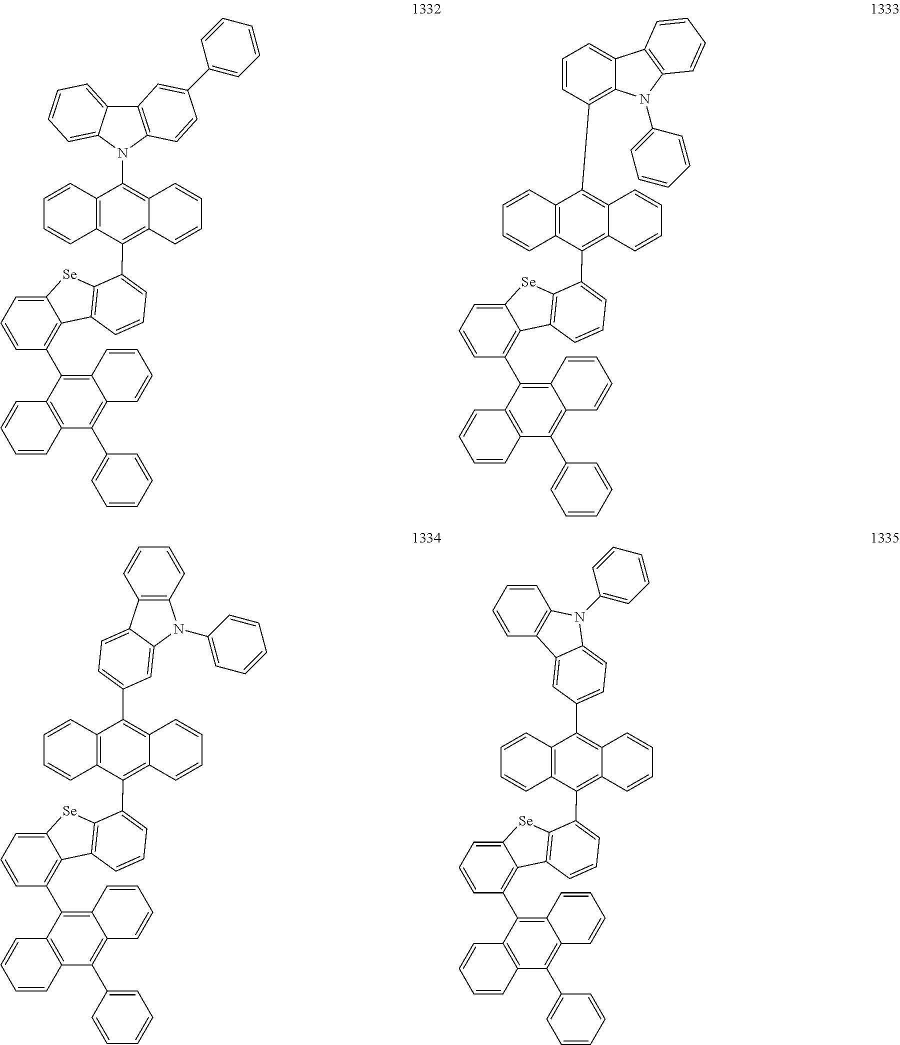

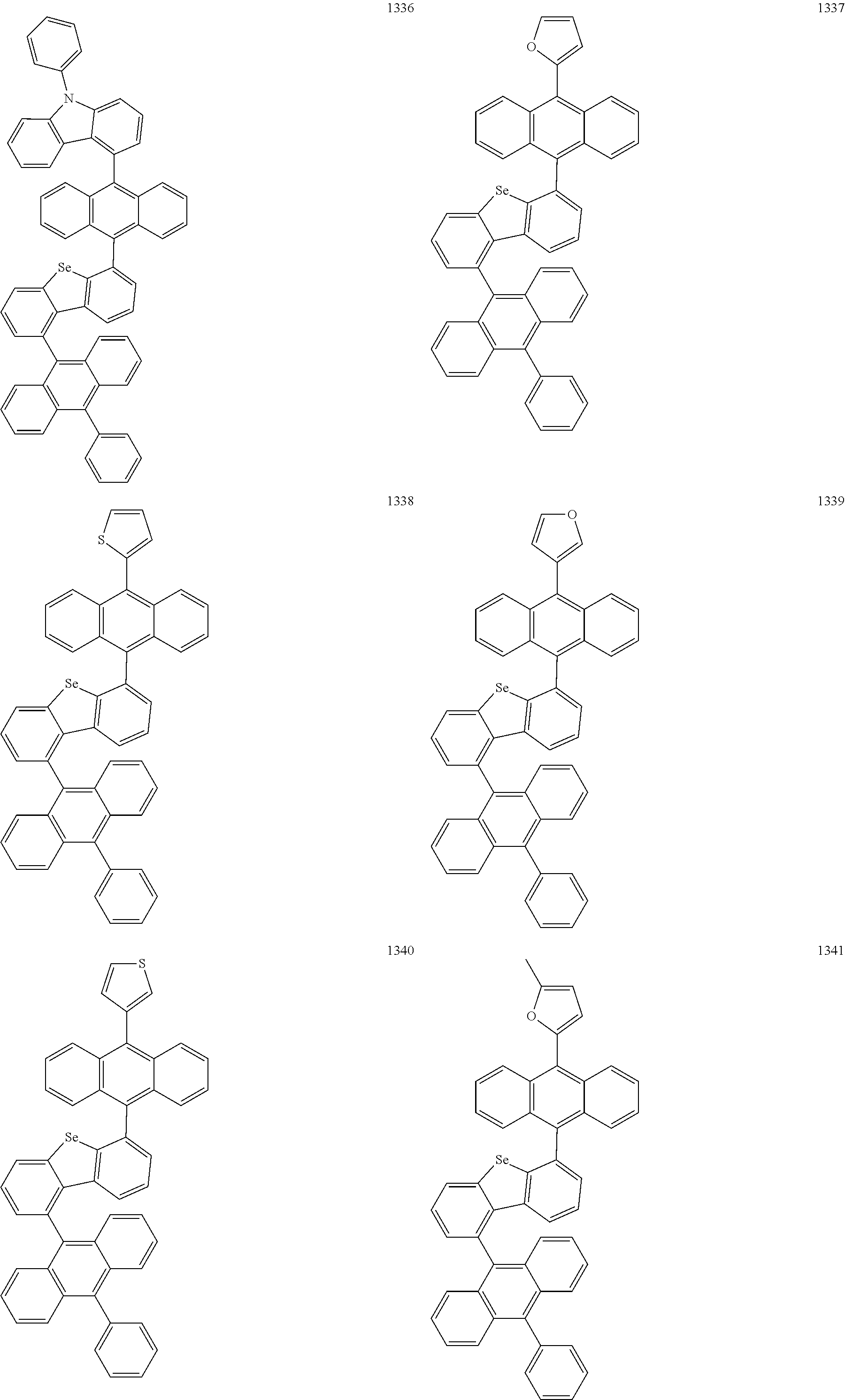

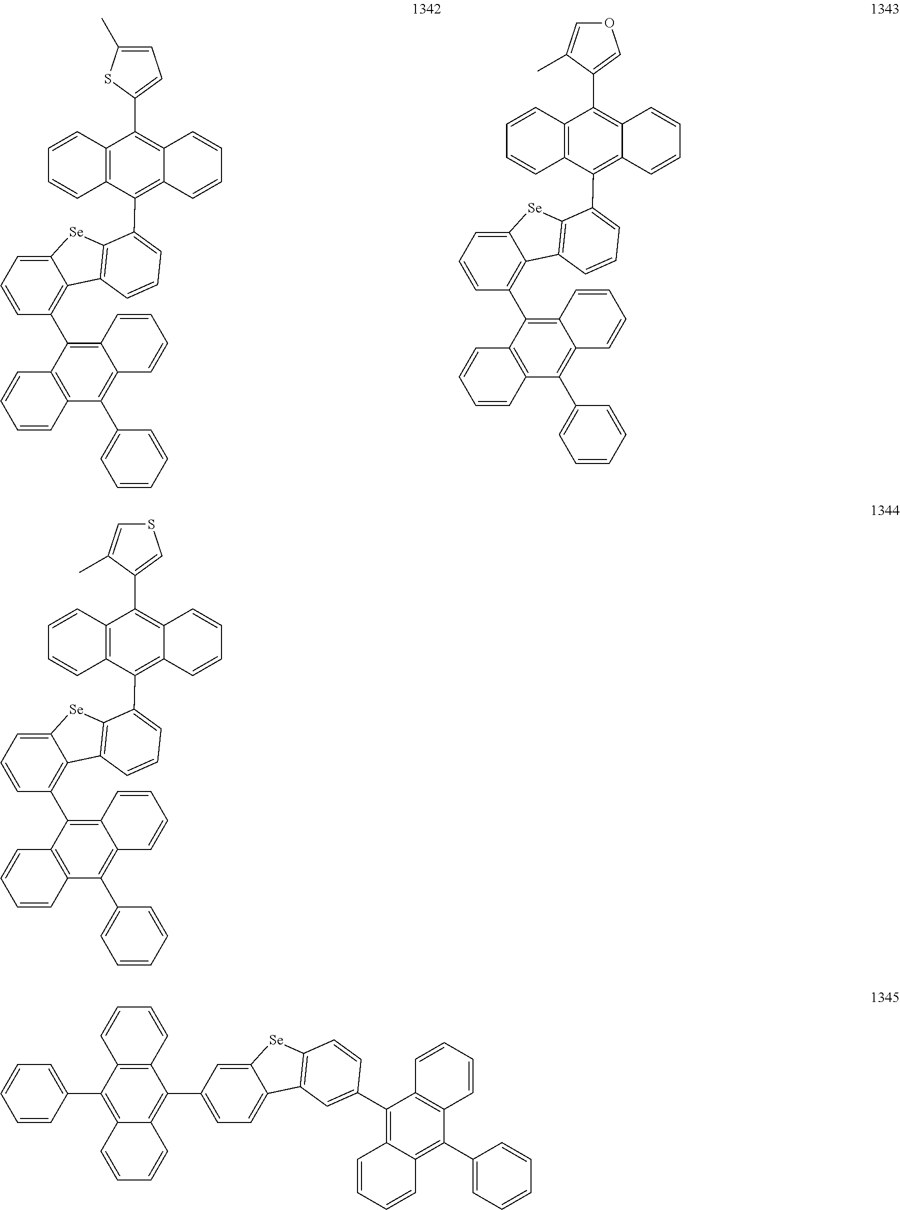

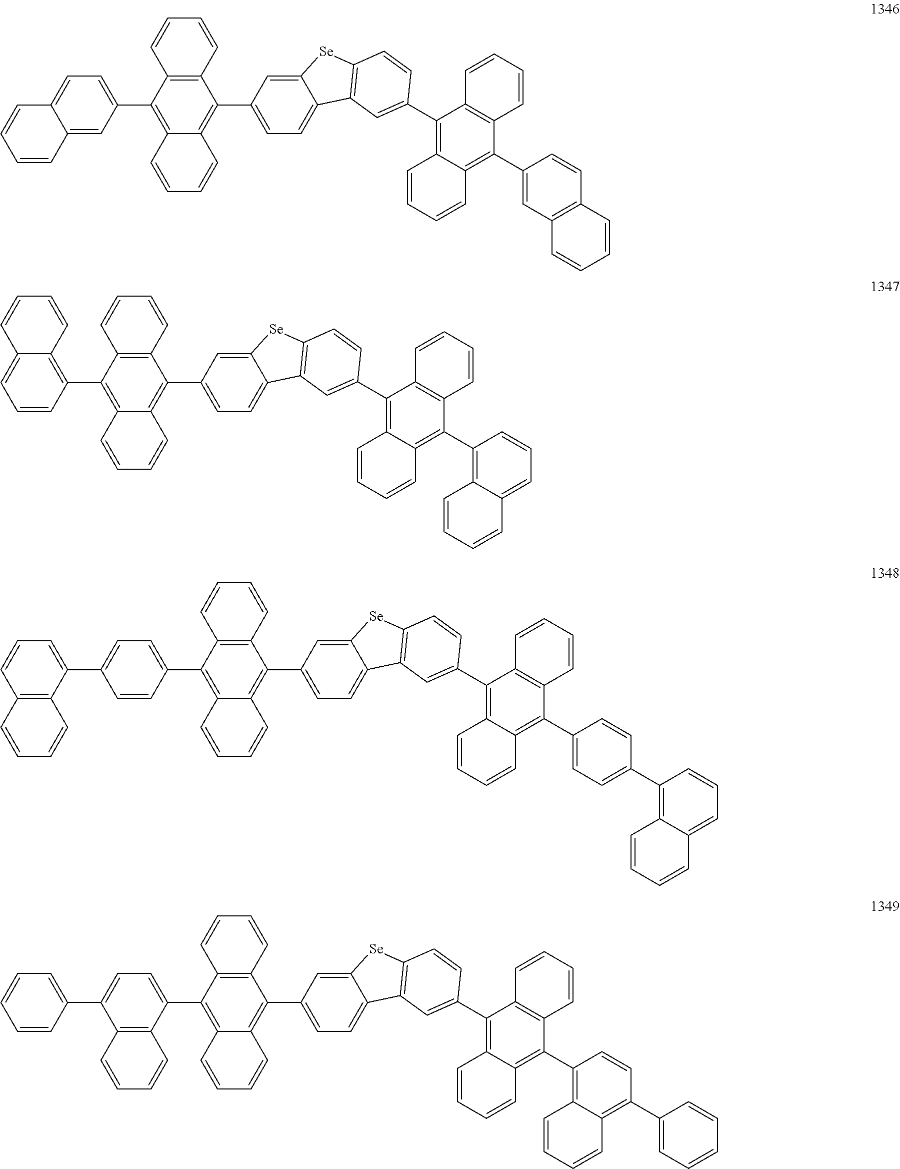 [SMILES: c1ccc2cc(-c3c4ccccc4c(-c4ccc5c(c4)[se]c4ccc(-c6c7ccccc7c(-c7ccc8ccccc8c7)c7ccccc67)cc45)c4ccccc34)ccc2c1 ; c1ccc2c(-c3c4ccccc4c(-c4ccc5c(c4)[se]c4ccc(-c6c7ccccc7c(-c7cccc8ccccc78)c7ccccc67)cc45)c4ccccc34)cccc2c1 ; c1ccc2c(-c3ccc(-c4c5ccccc5c(-c5ccc6c(c5)[se]c5ccc(-c7c8ccccc8c(-c8ccc(-c9cccc%10ccccc9%10)cc8)c8ccccc78)cc56)c5ccccc45)cc3)cccc2c1 ; c1ccc(-c2ccc(-c3c4ccccc4c(-c4ccc5c(c4)[se]c4ccc(-c6c7ccccc7c(-c7ccc(-c8ccccc8)c8ccccc78)c7ccccc67)cc45)c4ccccc34)c3ccccc23)cc1]

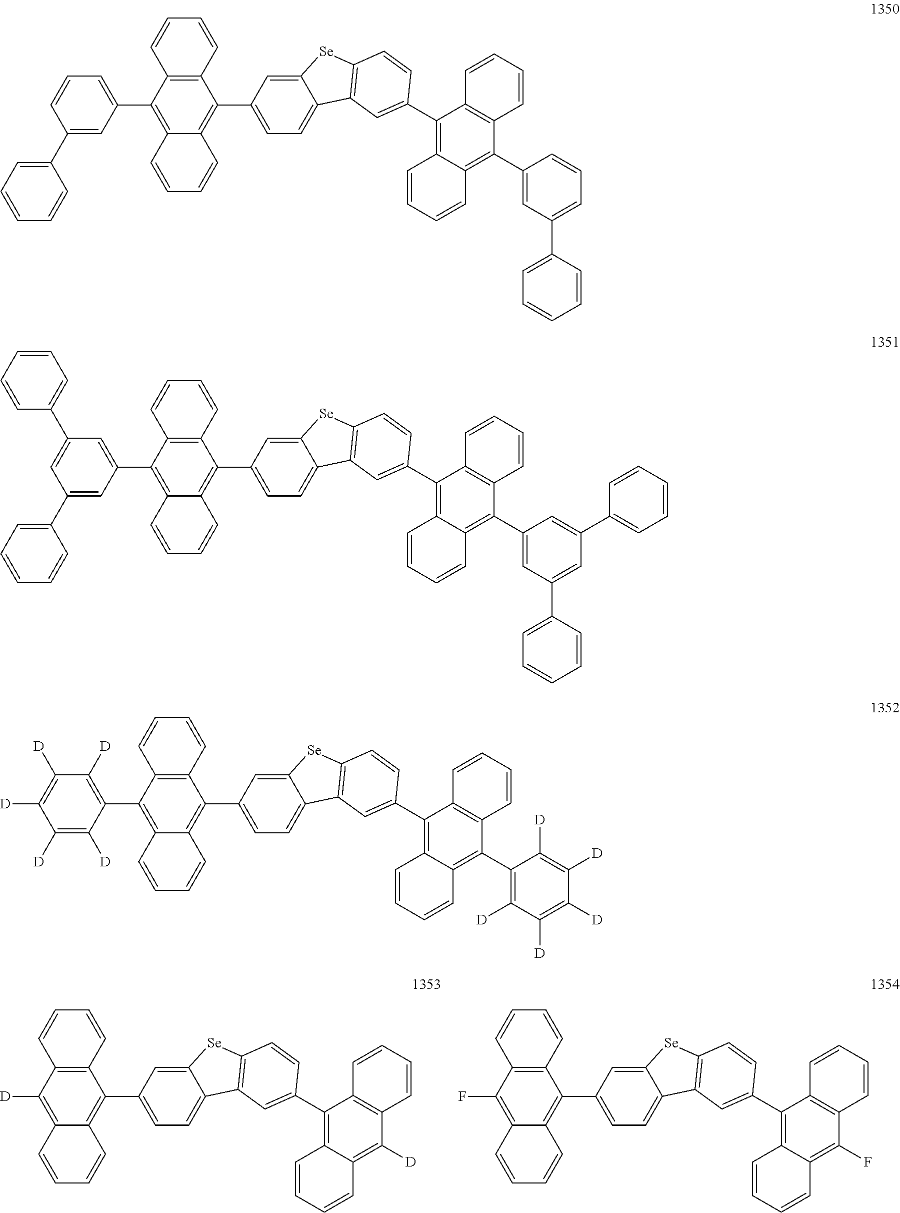

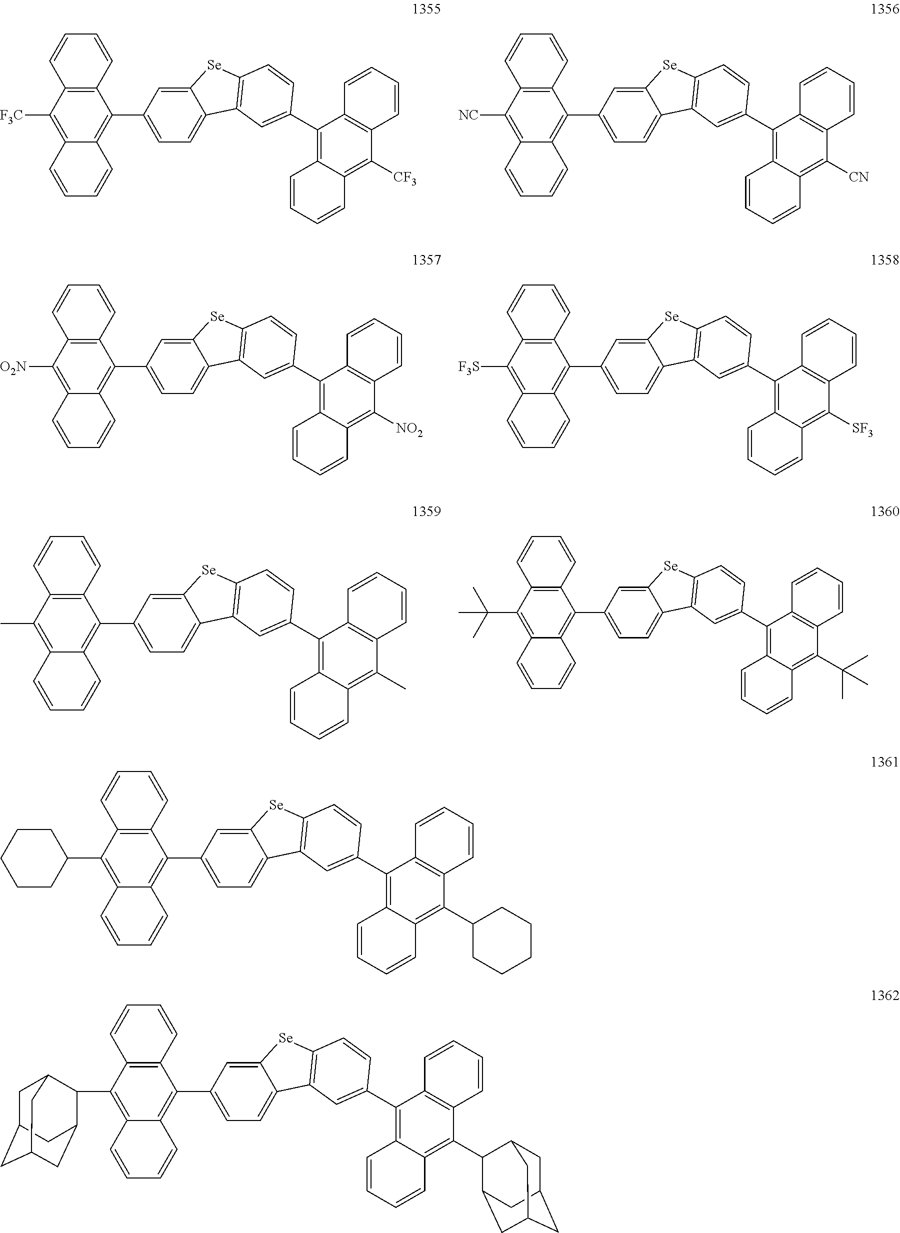

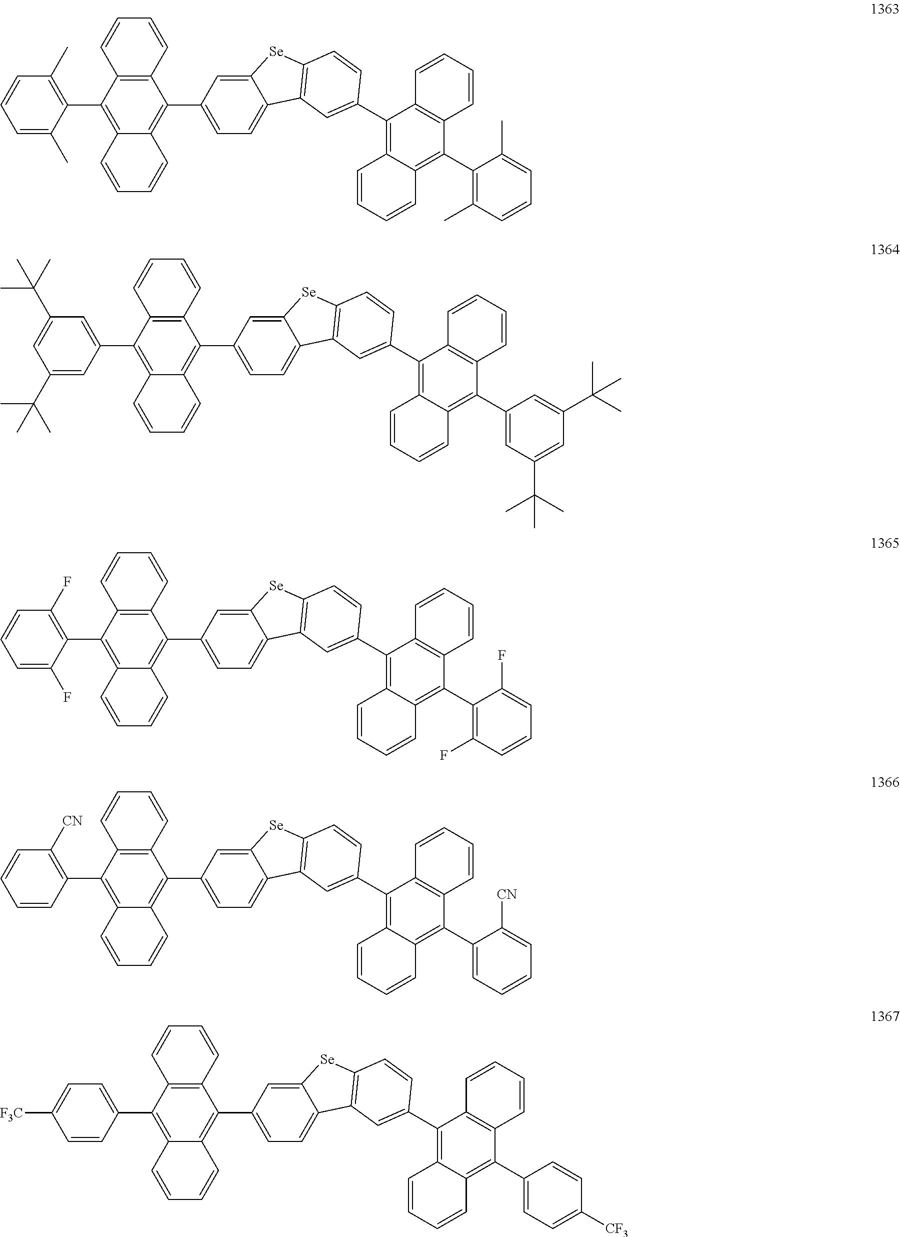

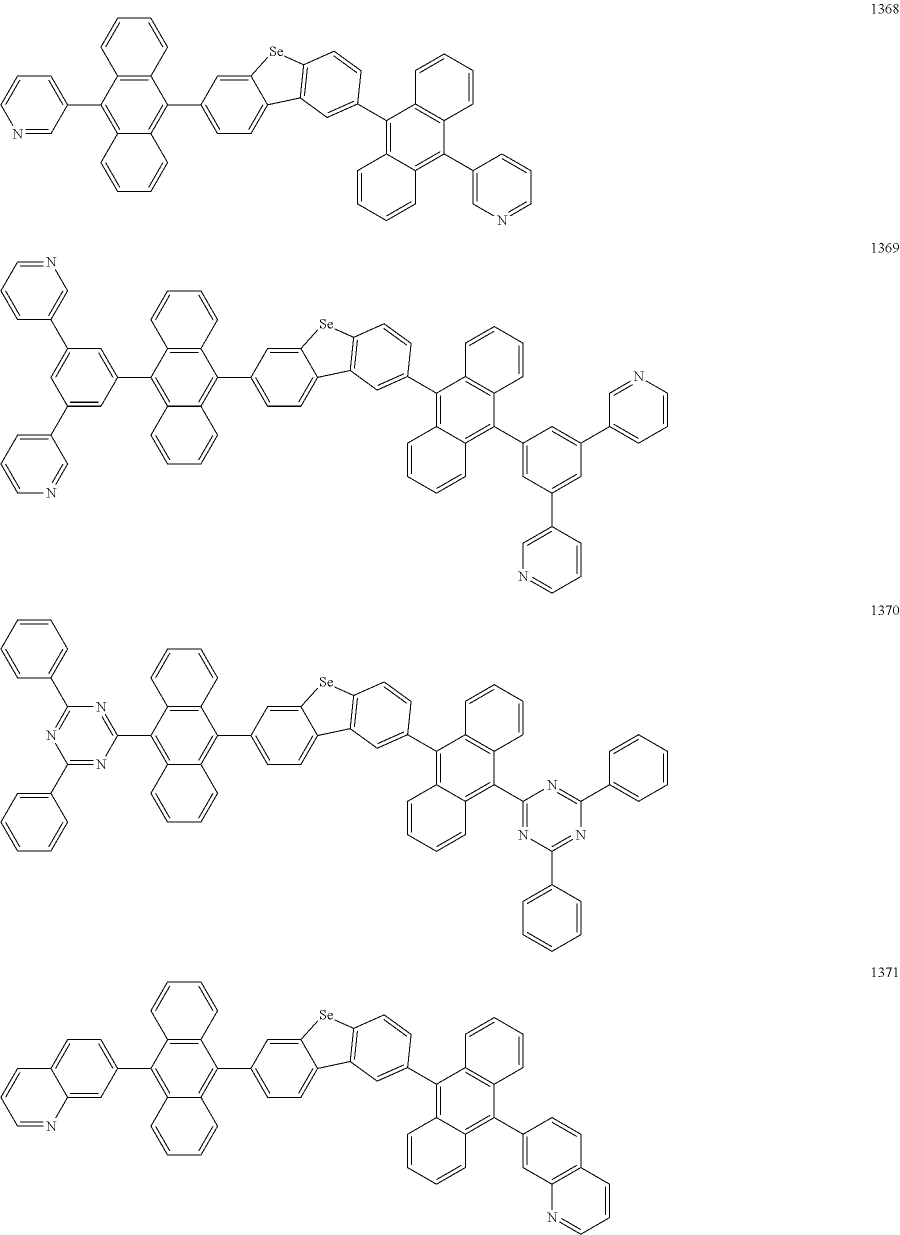 [SMILES: c1cncc(-c2c3ccccc3c(-c3ccc4c(c3)[se]c3ccc(-c5c6ccccc6c(-c6cccnc6)c6ccccc56)cc34)c3ccccc23)c1 ; c1cncc(-c2cc(-c3cccnc3)cc(-c3c4ccccc4c(-c4ccc5c(c4)[se]c4ccc(-c6c7ccccc7c(-c7cc(-c8cccnc8)cc(-c8cccnc8)c7)c7ccccc67)cc45)c4ccccc34)c2)c1 ; c1ccc(-c2nc(-c3ccccc3)nc(-c3c4ccccc4c(-c4ccc5c(c4)[se]c4ccc(-c6c7ccccc7c(-c7nc(-c8ccccc8)nc(-c8ccccc8)n7)c7ccccc67)cc45)c4ccccc34)n2)cc1 ; c1cnc2cc(-c3c4ccccc4c(-c4ccc5c(c4)[se]c4ccc(-c6c7ccccc7c(-c7ccc8cccnc8c7)c7ccccc67)cc45)c4ccccc34)ccc2c1]

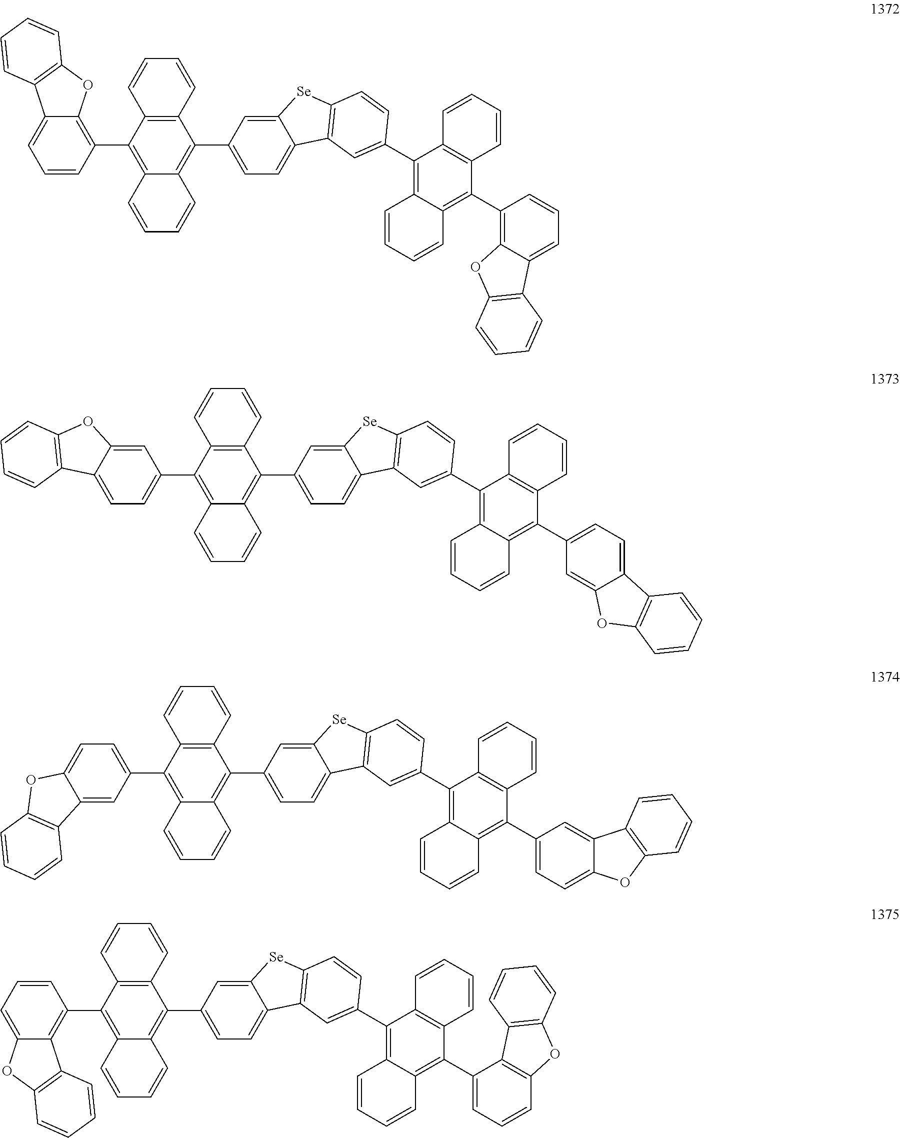 [SMILES: c1ccc2c(c1)oc1c(-c3c4ccccc4c(-c4ccc5c(c4)[se]c4ccc(-c6c7ccccc7c(-c7cccc8c7oc7ccccc78)c7ccccc67)cc45)c4ccccc34)cccc12 ; c1ccc2c(c1)oc1cc(-c3c4ccccc4c(-c4ccc5c(c4)[se]c4ccc(-c6c7ccccc7c(-c7ccc8c(c7)oc7ccccc78)c7ccccc67)cc45)c4ccccc34)ccc12 ; c1ccc2c(c1)oc1ccc(-c3c4ccccc4c(-c4ccc5c(c4)[se]c4ccc(-c6c7ccccc7c(-c7ccc8oc9ccccc9c8c7)c7ccccc67)cc45)c4ccccc34)cc12 ; c1ccc2c(c1)oc1cccc(-c3c4ccccc4c(-c4ccc5c(c4)[se]c4ccc(-c6c7ccccc7c(-c7cccc8oc9ccccc9c78)c7ccccc67)cc45)c4ccccc34)c12]

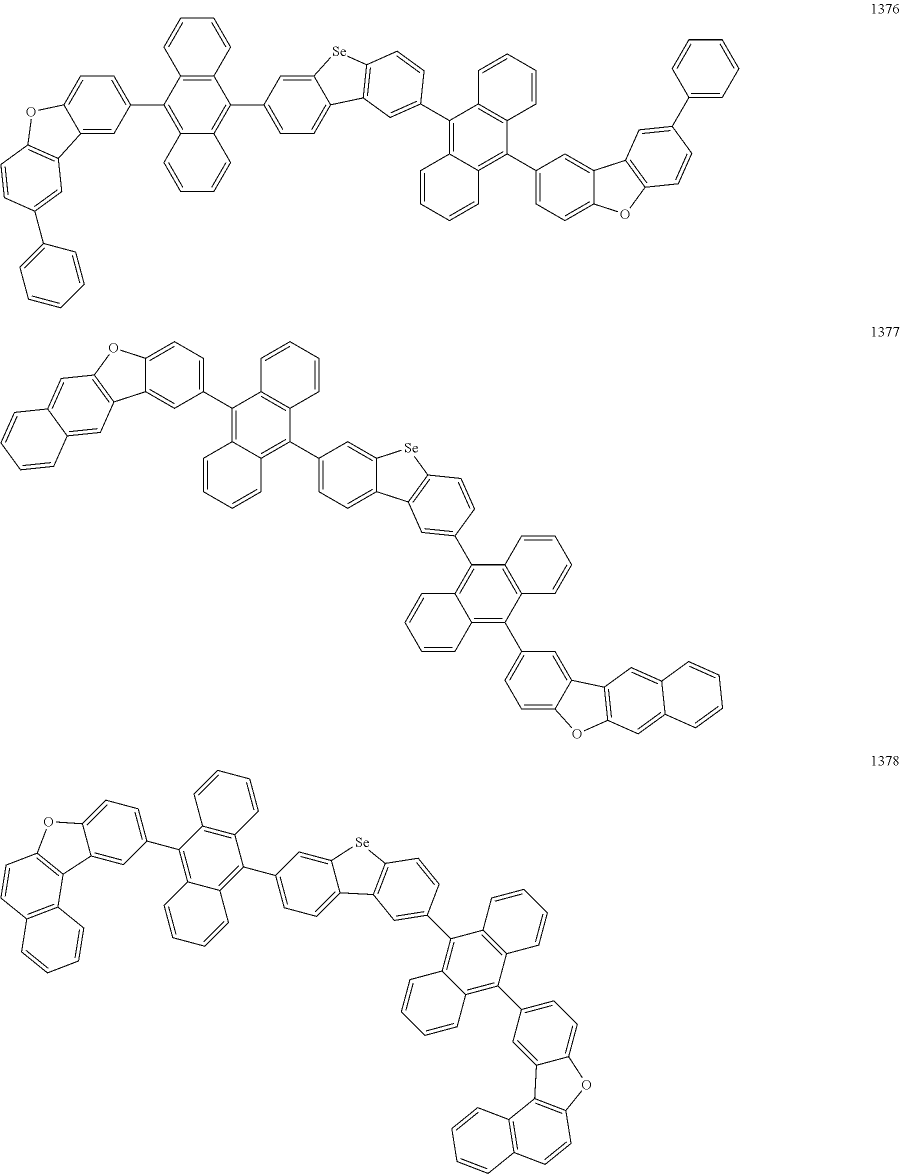 [SMILES: c1ccc(-c2ccc3oc4ccc(-c5c6ccccc6c(-c6ccc7c(c6)[se]c6ccc(-c8c9ccccc9c(-c9ccc%10oc%11ccc(-c%12ccccc%12)cc%11c%10c9)c9ccccc89)cc67)c6ccccc56)cc4c3c2)cc1 ; c1ccc2cc3c(cc2c1)oc1ccc(-c2c4ccccc4c(-c4ccc5c(c4)[se]c4ccc(-c6c7ccccc7c(-c7ccc8oc9cc%10ccccc%10cc9c8c7)c7ccccc67)cc45)c4ccccc24)cc13 ; c1ccc2c(c1)ccc1oc3ccc(-c4c5ccccc5c(-c5ccc6c(c5)[se]c5ccc(-c7c8ccccc8c(-c8ccc9oc%10ccc%11ccccc%11c%10c9c8)c8ccccc78)cc56)c5ccccc45)cc3c12]

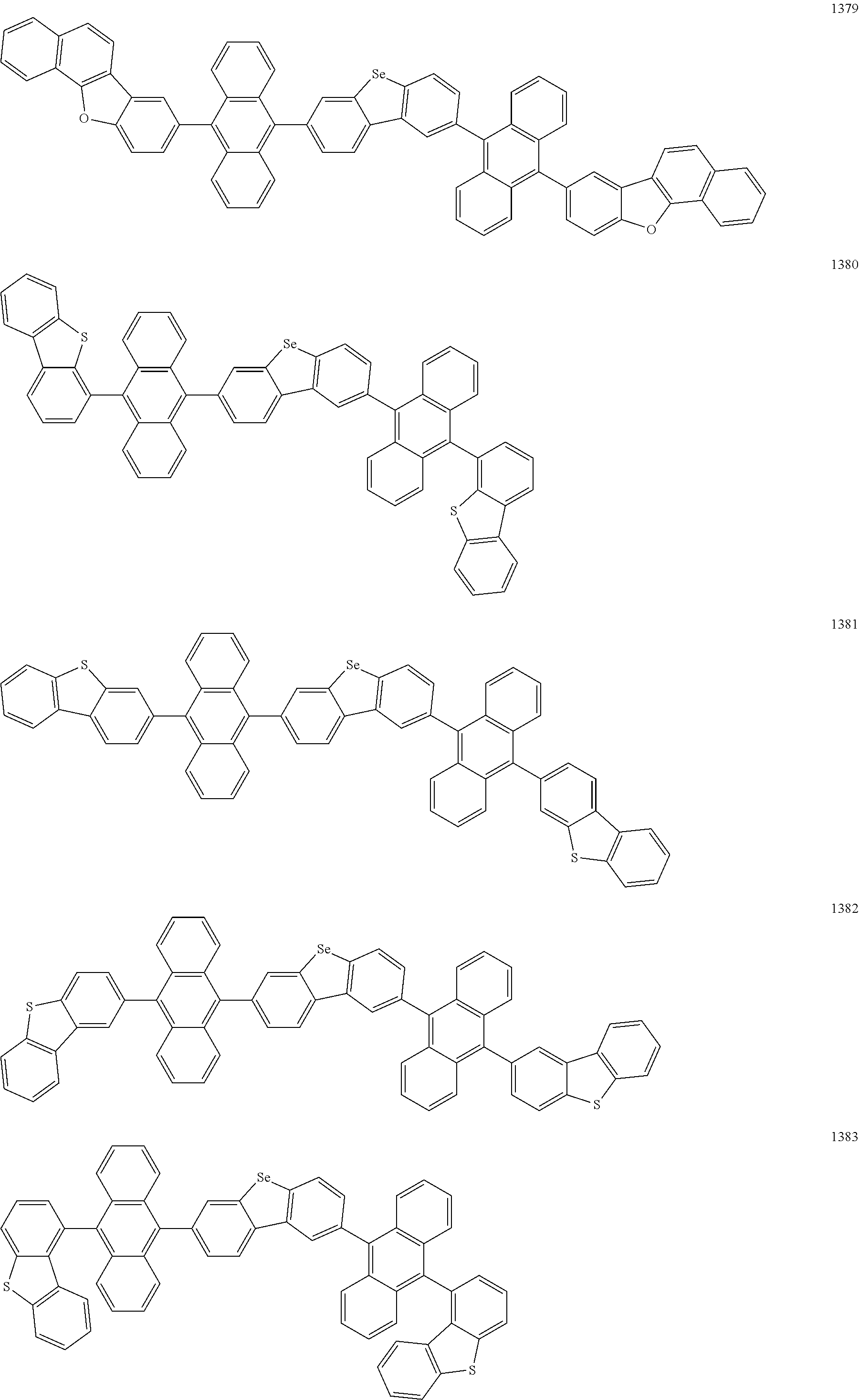

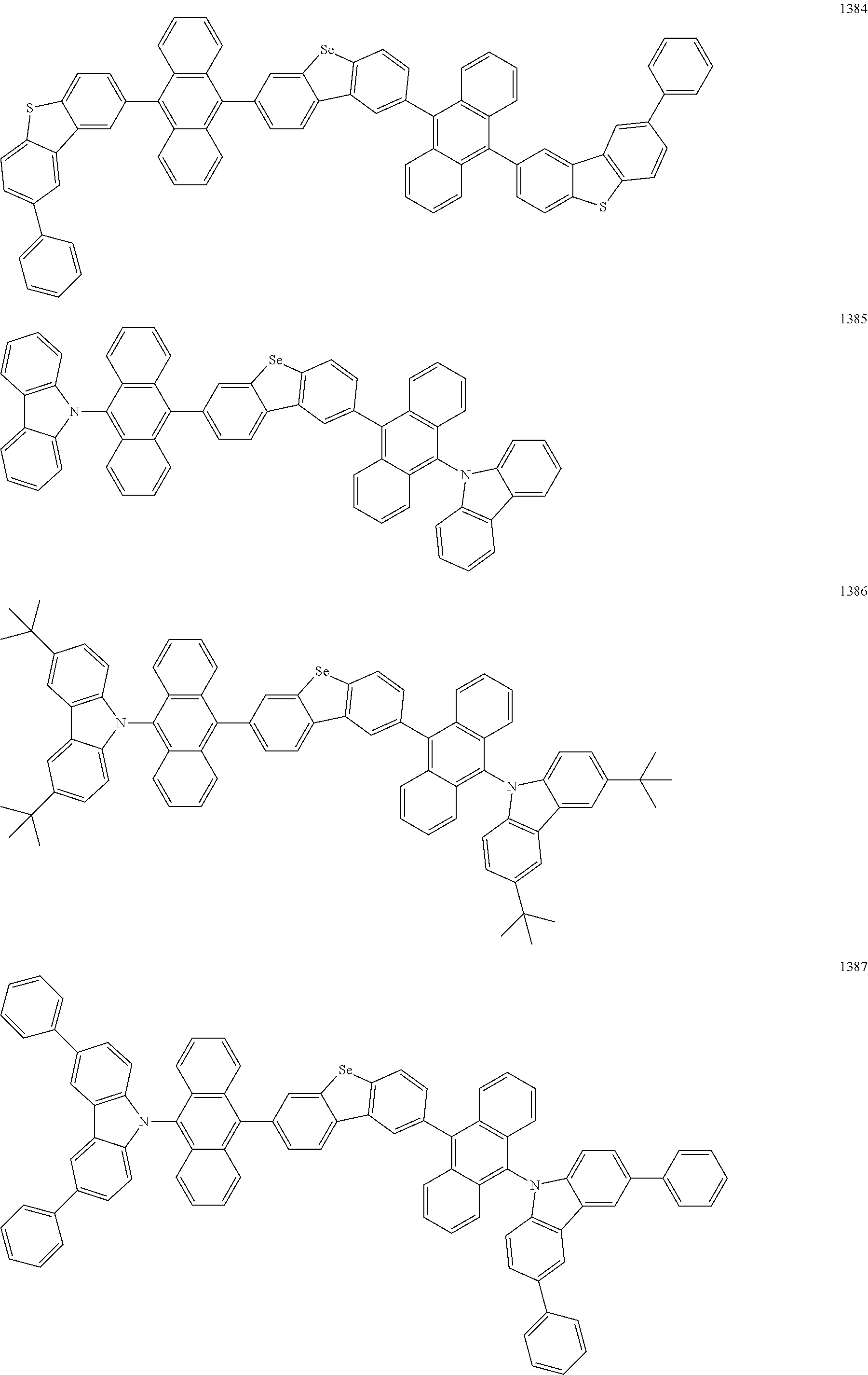

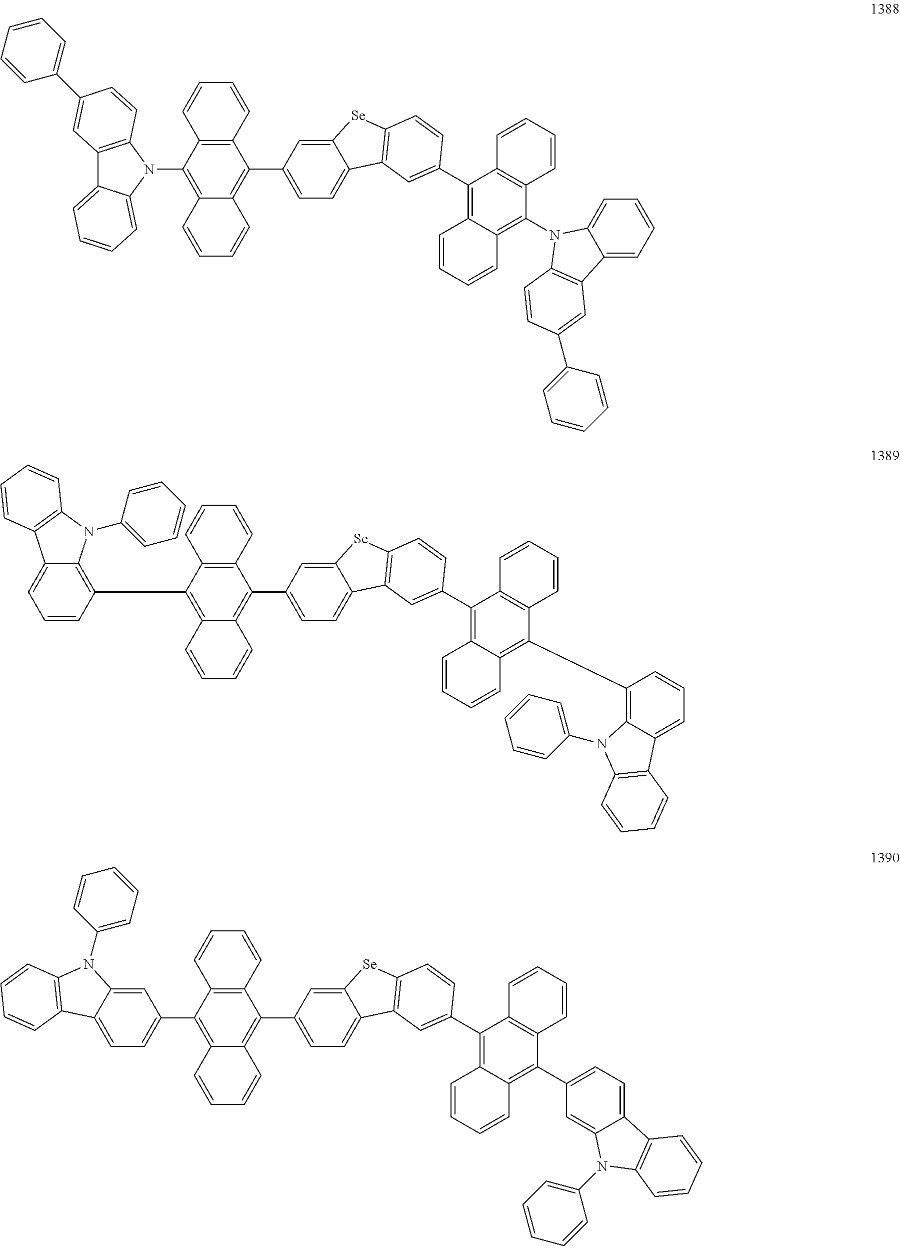 [SMILES: c1ccc(-c2ccc3c(c2)c2ccccc2n3-c2c3ccccc3c(-c3ccc4c(c3)[se]c3ccc(-c5c6ccccc6c(-n6c7ccccc7c7cc(-c8ccccc8)ccc76)c6ccccc56)cc34)c3ccccc23)cc1 ; c1ccc(-n2c3ccccc3c3cccc(-c4c5ccccc5c(-c5ccc6c(c5)[se]c5ccc(-c7c8ccccc8c(-c8cccc9c%10ccccc%10n(-c%10ccccc%10)c89)c8ccccc78)cc56)c5ccccc45)c32)cc1 ; c1ccc(-n2c3ccccc3c3ccc(-c4c5ccccc5c(-c5ccc6c(c5)[se]c5ccc(-c7c8ccccc8c(-c8ccc9c%10ccccc%10n(-c%10ccccc%10)c9c8)c8ccccc78)cc56)c5ccccc45)cc32)cc1]

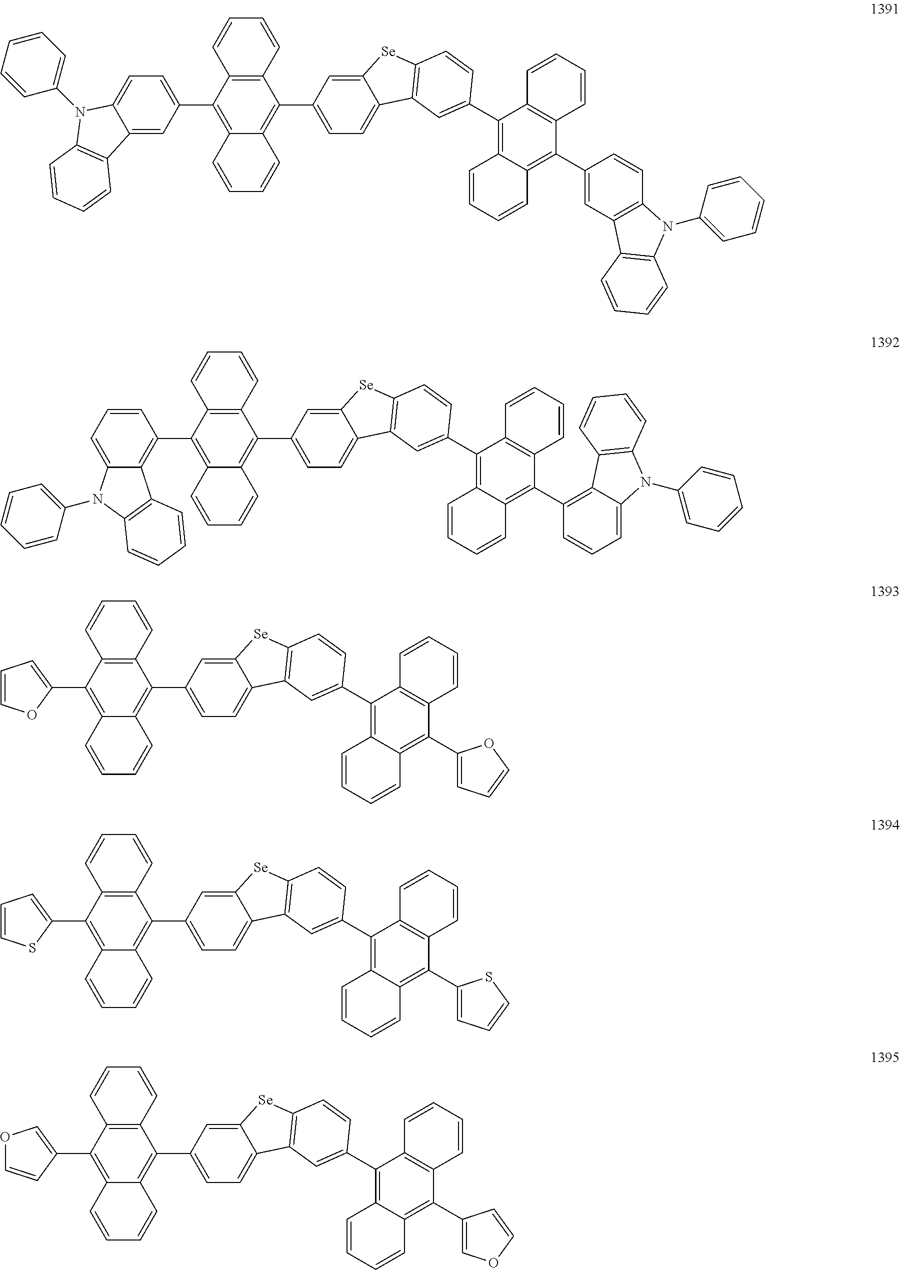

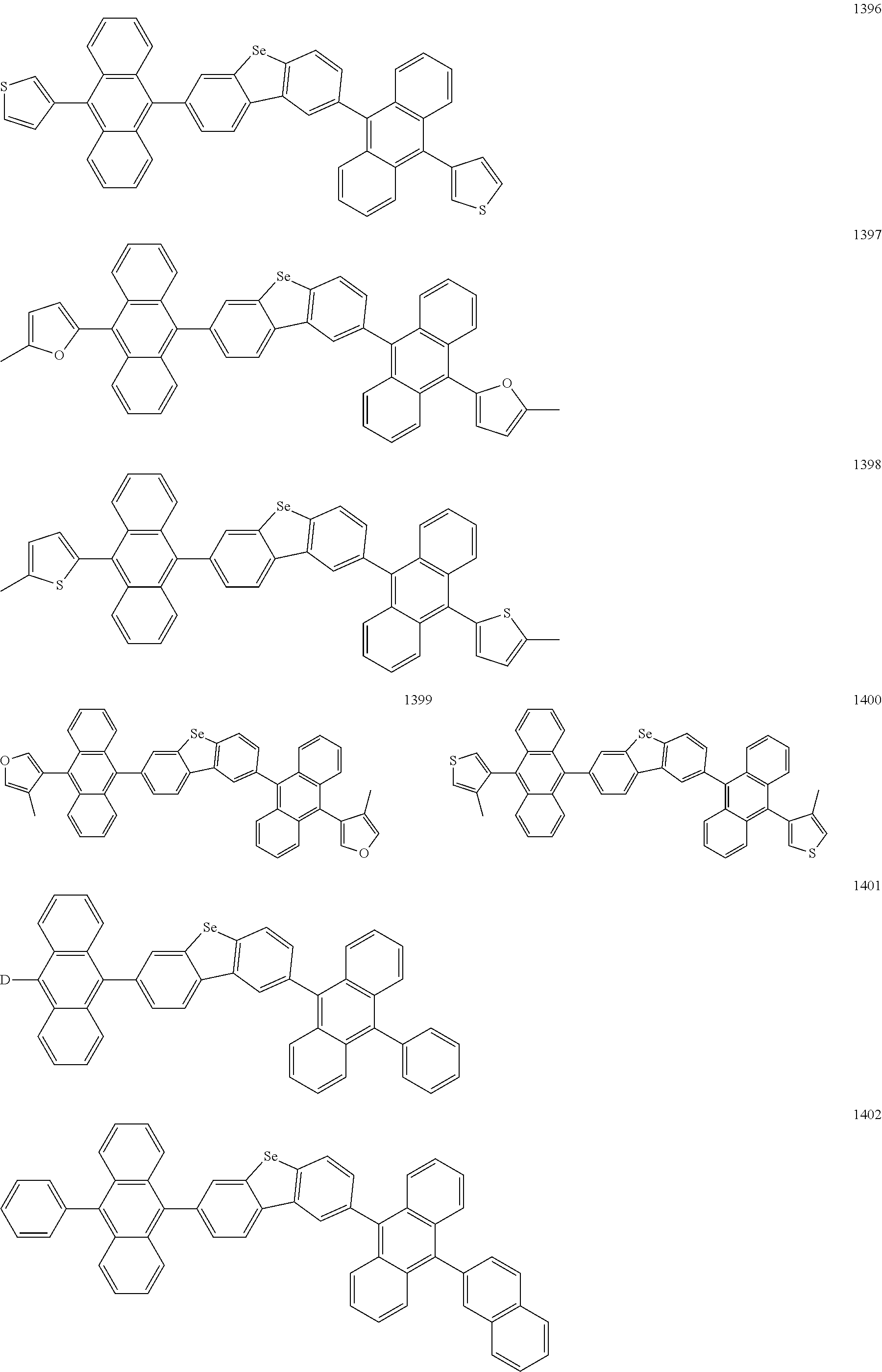

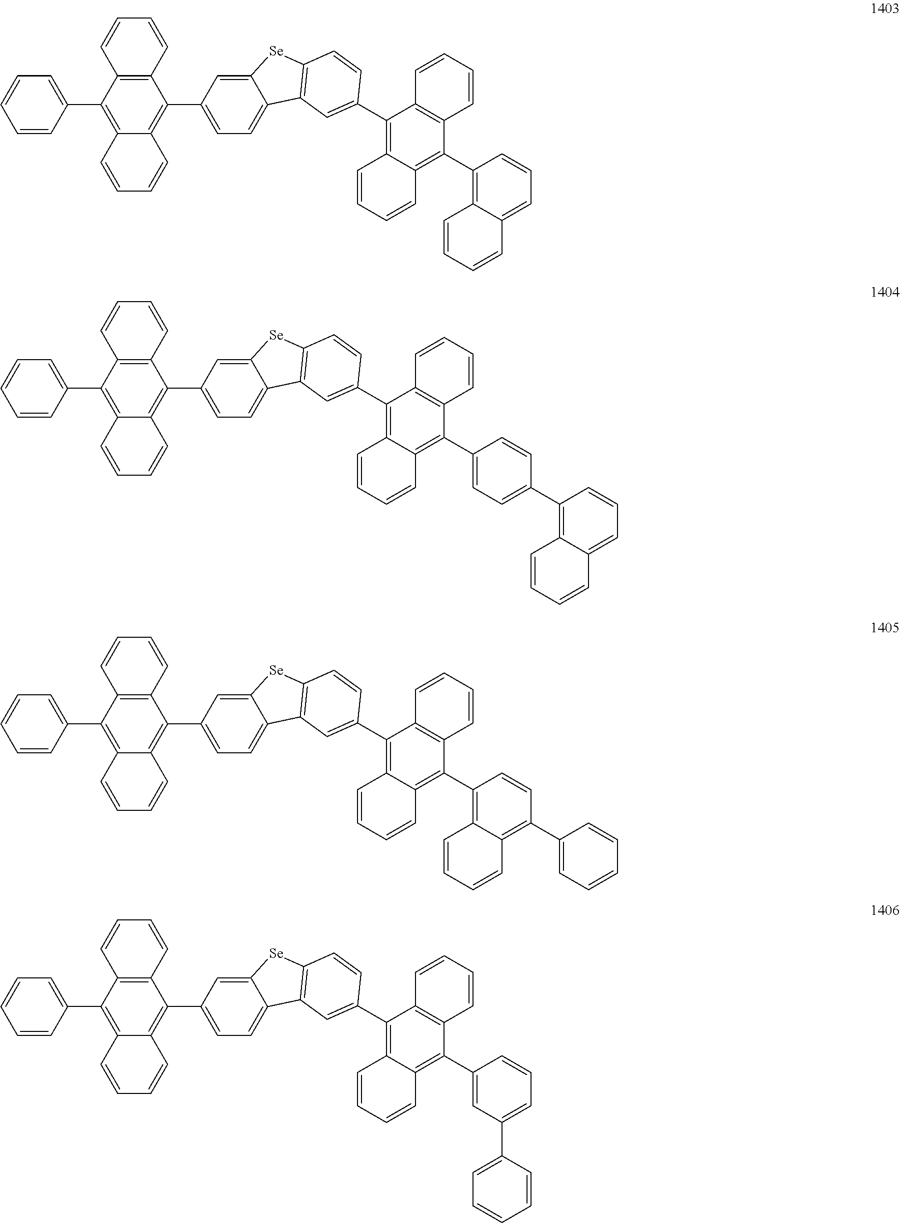 [SMILES: c1ccc(-c2c3ccccc3c(-c3ccc4c(c3)[se]c3ccc(-c5c6ccccc6c(-c6cccc7ccccc67)c6ccccc56)cc34)c3ccccc23)cc1 ; c1ccc(-c2c3ccccc3c(-c3ccc4c(c3)[se]c3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5[se]c6cc(-c7c8ccccc8c(-c8ccccc8)c8ccccc78)ccc6c5c4)c4ccccc34)c3ccccc23)cc1 ; c1ccc(-c2cccc(-c3c4ccccc4c(-c4ccc5[se]c6cc(-c7c8ccccc8c(-c8ccccc8)c8ccccc78)ccc6c5c4)c4ccccc34)c2)cc1]

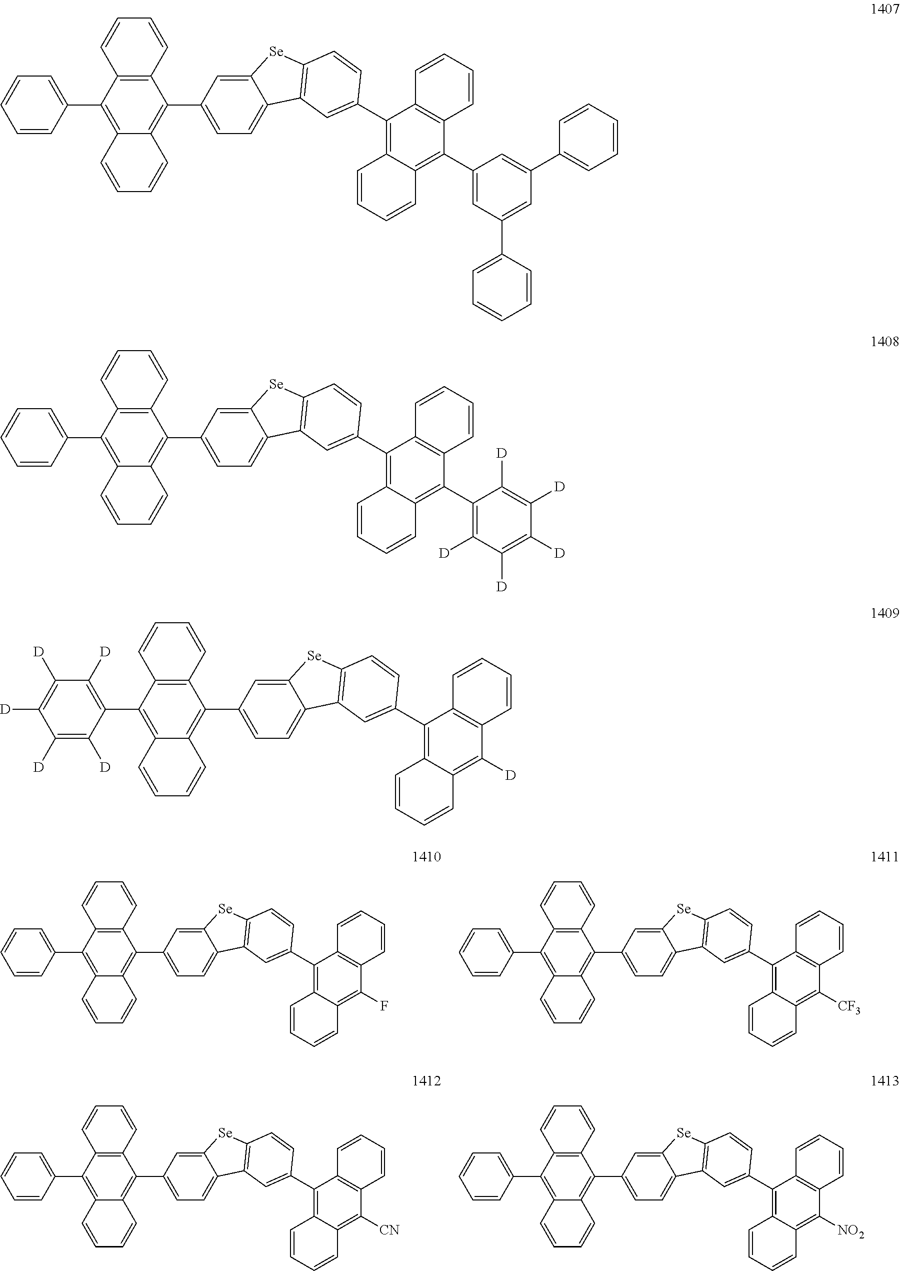

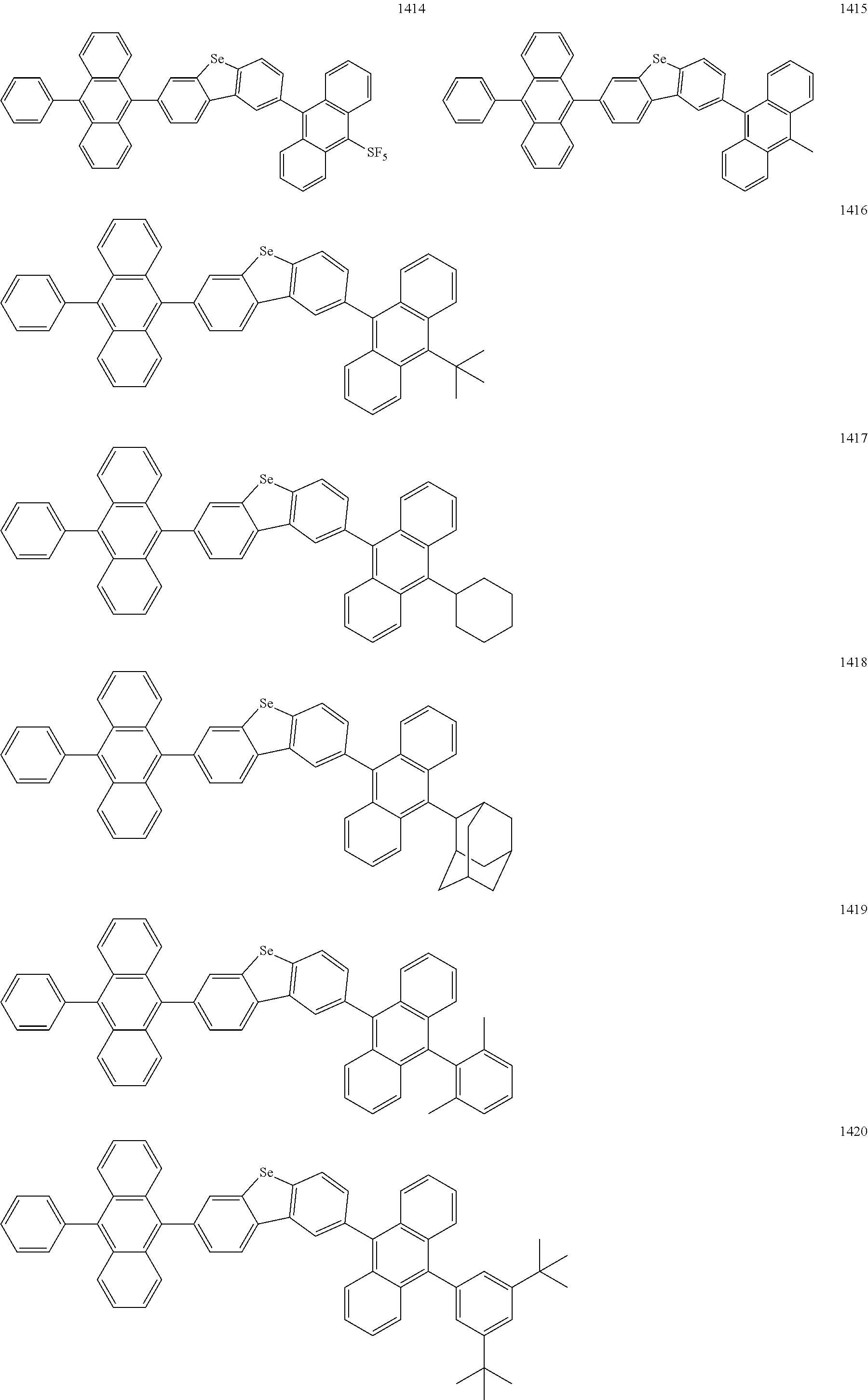

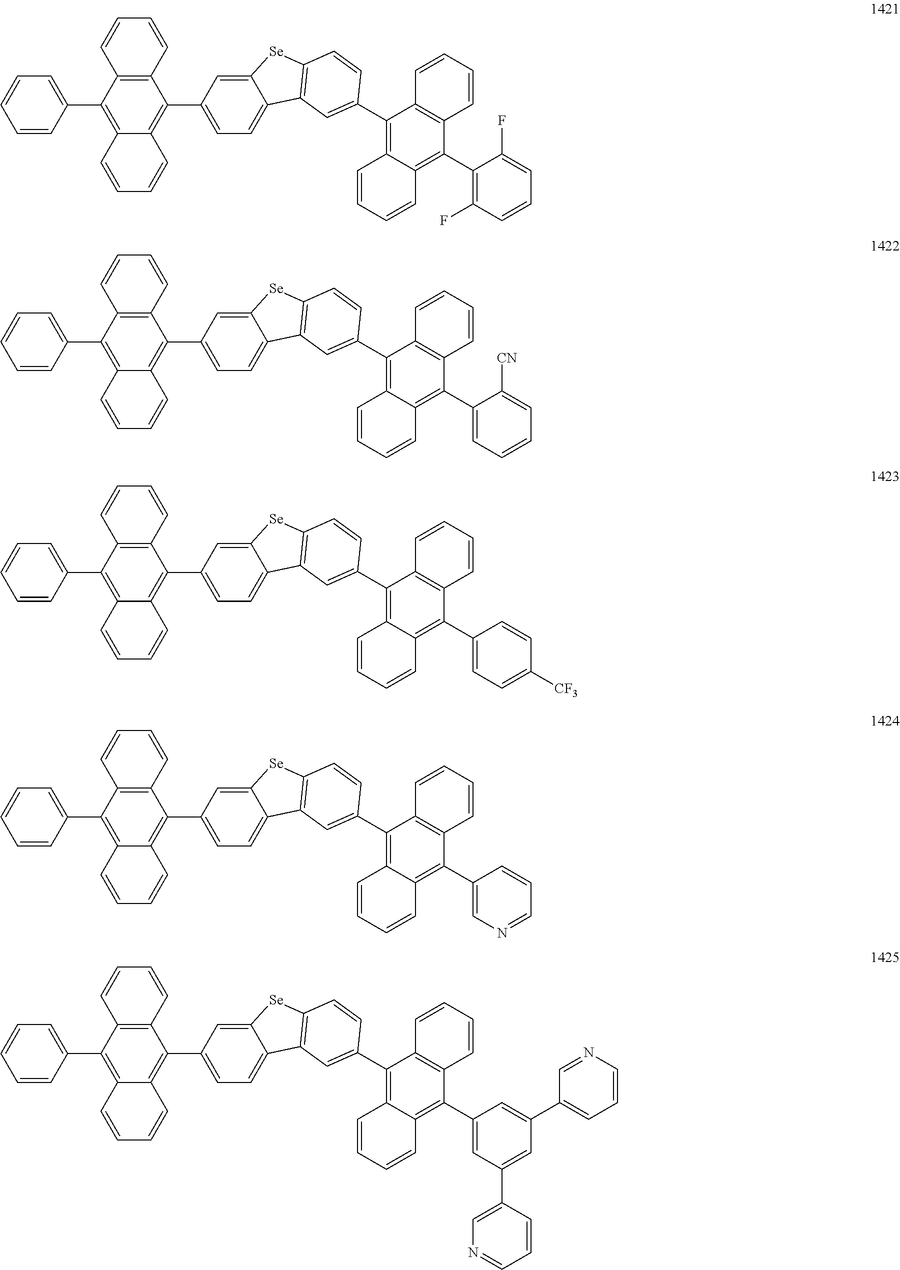 [SMILES: Fc1cccc(F)c1-c1c2ccccc2c(-c2ccc3[se]c4cc(-c5c6ccccc6c(-c6ccccc6)c6ccccc56)ccc4c3c2)c2ccccc12 ; N#Cc1ccccc1-c1c2ccccc2c(-c2ccc3[se]c4cc(-c5c6ccccc6c(-c6ccccc6)c6ccccc56)ccc4c3c2)c2ccccc12 ; FC(F)(F)c1ccc(-c2c3ccccc3c(-c3ccc4[se]c5cc(-c6c7ccccc7c(-c7ccccc7)c7ccccc67)ccc5c4c3)c3ccccc23)cc1 ; c1ccc(-c2c3ccccc3c(-c3ccc4c(c3)[se]c3ccc(-c5c6ccccc6c(-c6cccnc6)c6ccccc56)cc34)c3ccccc23)cc1 ; c1ccc(-c2c3ccccc3c(-c3ccc4c(c3)[se]c3ccc(-c5c6ccccc6c(-c6cc(-c7cccnc7)cc(-c7cccnc7)c6)c6ccccc56)cc34)c3ccccc23)cc1]

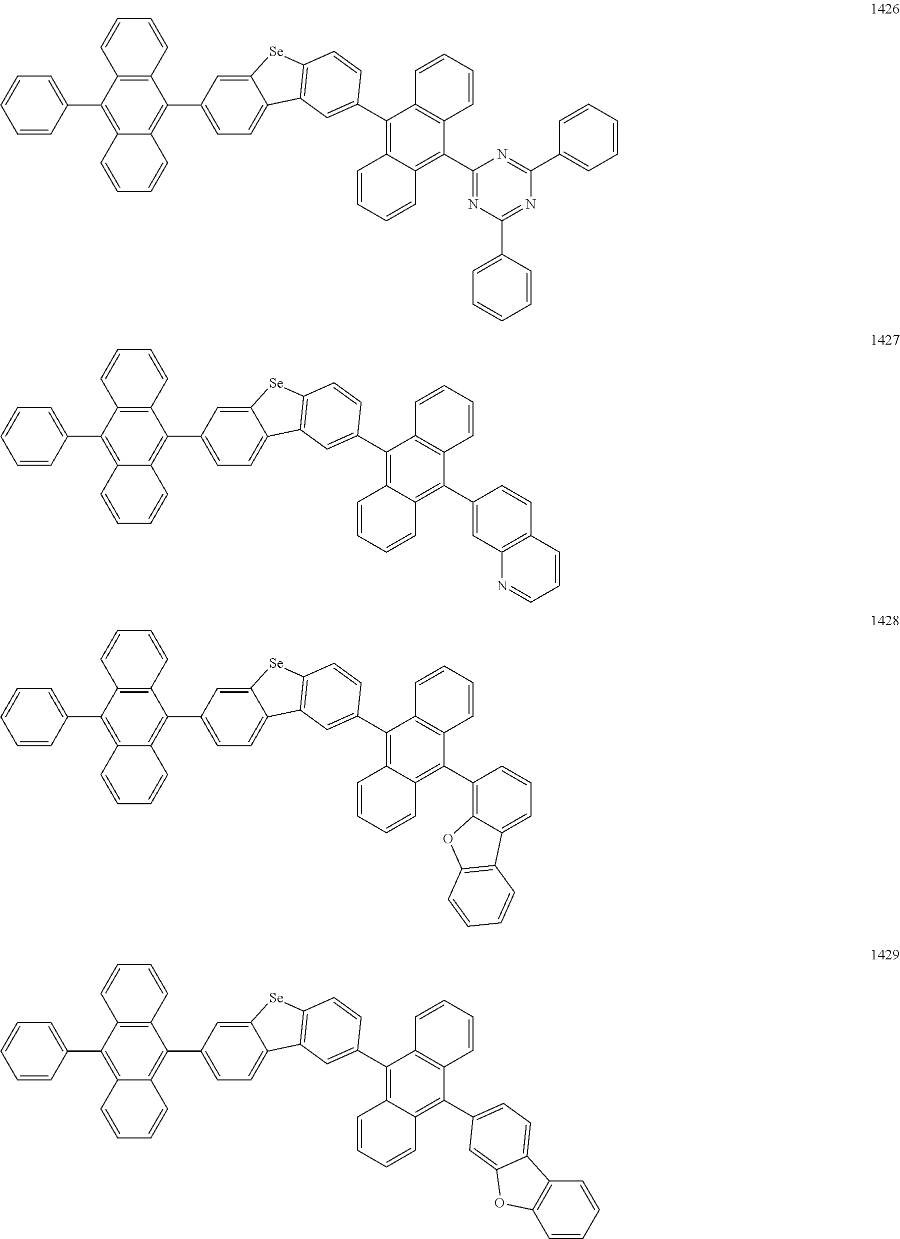 [SMILES: c1ccc(-c2nc(-c3ccccc3)nc(-c3c4ccccc4c(-c4ccc5[se]c6cc(-c7c8ccccc8c(-c8ccccc8)c8ccccc78)ccc6c5c4)c4ccccc34)n2)cc1 ; c1ccc(-c2c3ccccc3c(-c3ccc4c(c3)[se]c3ccc(-c5c6ccccc6c(-c6ccc7cccnc7c6)c6ccccc56)cc34)c3ccccc23)cc1 ; c1ccc(-c2c3ccccc3c(-c3ccc4c(c3)[se]c3ccc(-c5c6ccccc6c(-c6cccc7c6oc6ccccc67)c6ccccc56)cc34)c3ccccc23)cc1 ; c1ccc(-c2c3ccccc3c(-c3ccc4c(c3)[se]c3ccc(-c5c6ccccc6c(-c6ccc7c(c6)oc6ccccc67)c6ccccc56)cc34)c3ccccc23)cc1]

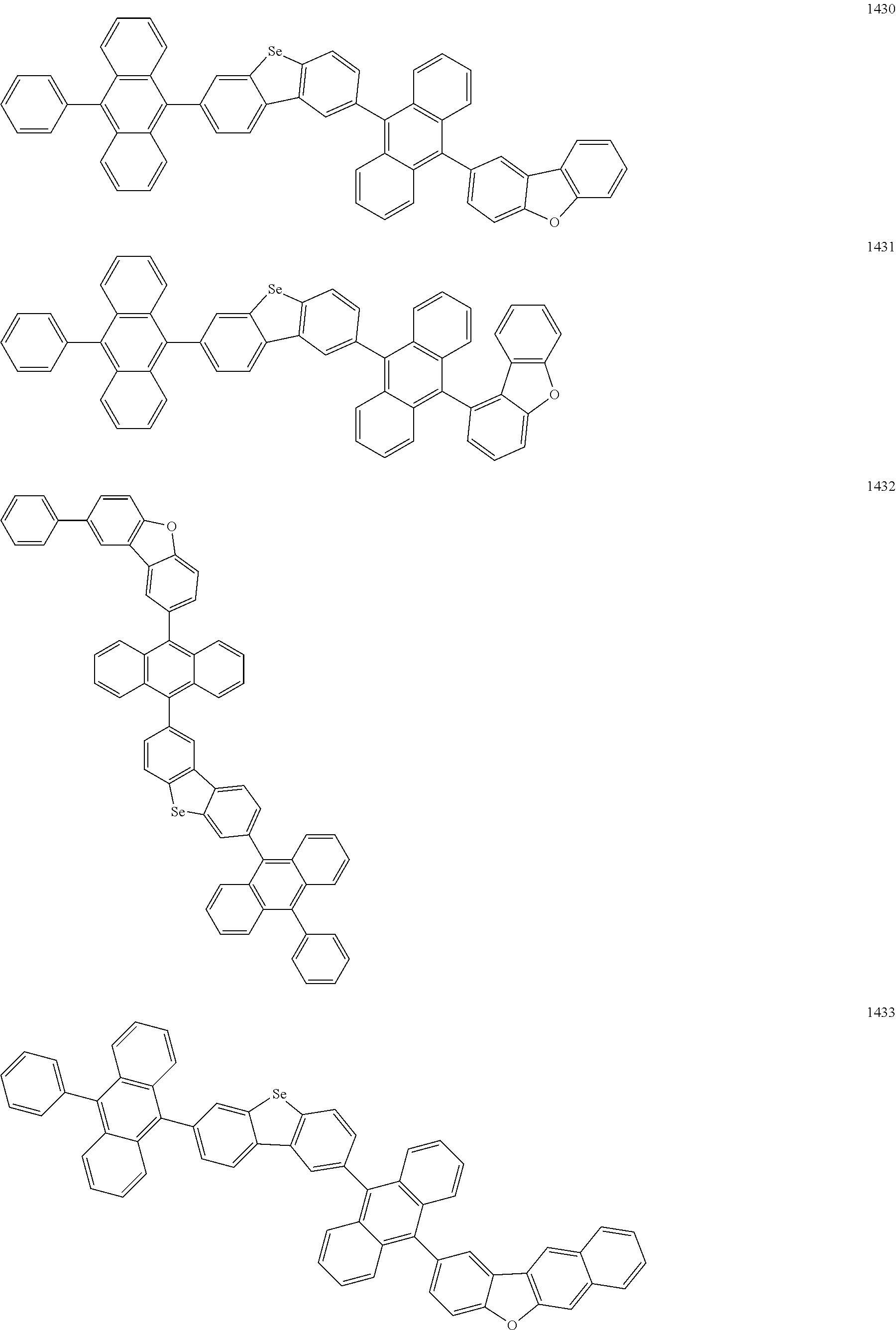 [SMILES: c1ccc(-c2c3ccccc3c(-c3ccc4c(c3)[se]c3ccc(-c5c6ccccc6c(-c6ccc7oc8ccccc8c7c6)c6ccccc56)cc34)c3ccccc23)cc1 ; c1ccc(-c2c3ccccc3c(-c3ccc4c(c3)[se]c3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7[se]c8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)[se]c3ccc(-c5c6ccccc6c(-c6ccc7oc8cc9ccccc9cc8c7c6)c6ccccc56)cc34)c3ccccc23)cc1]

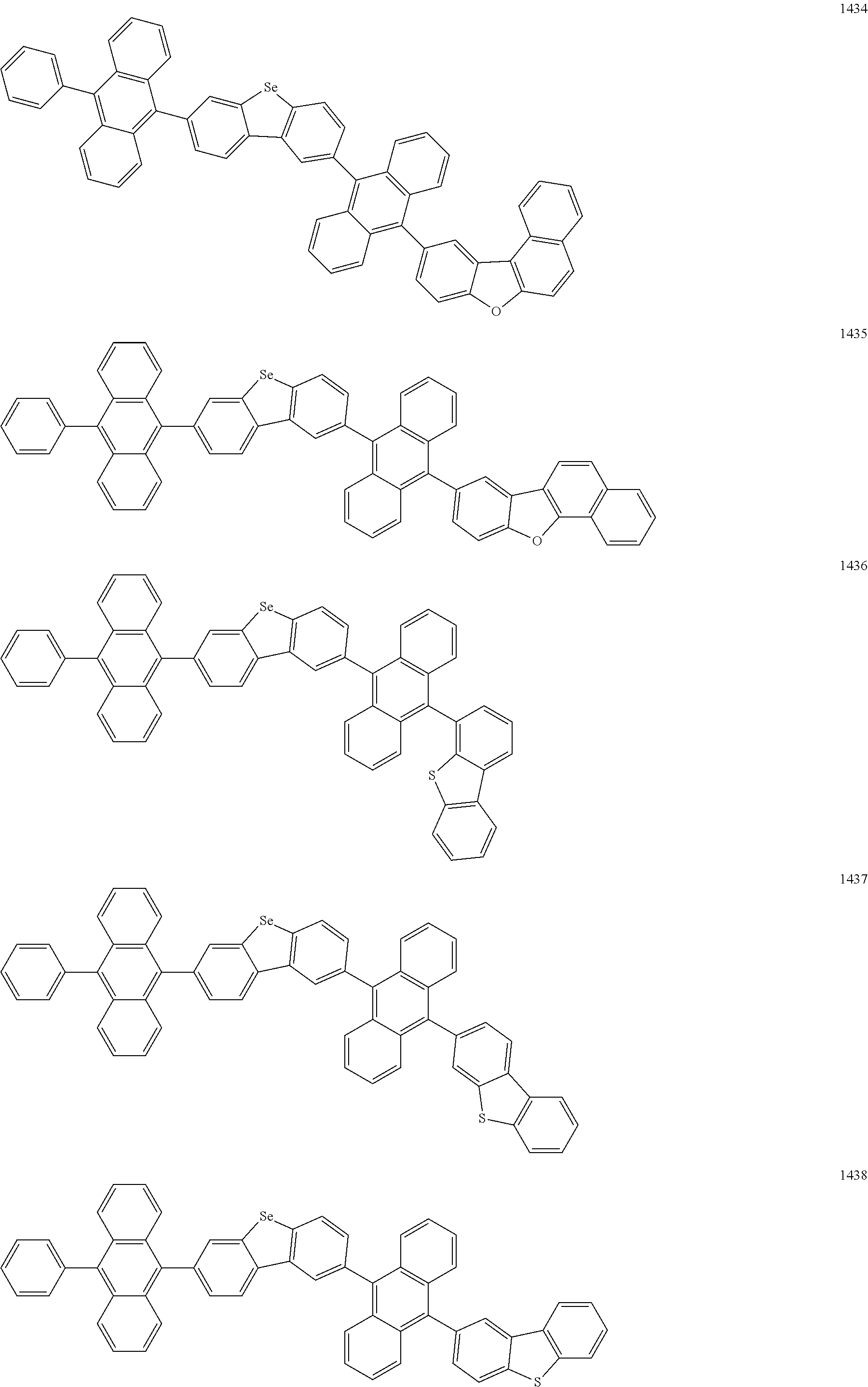

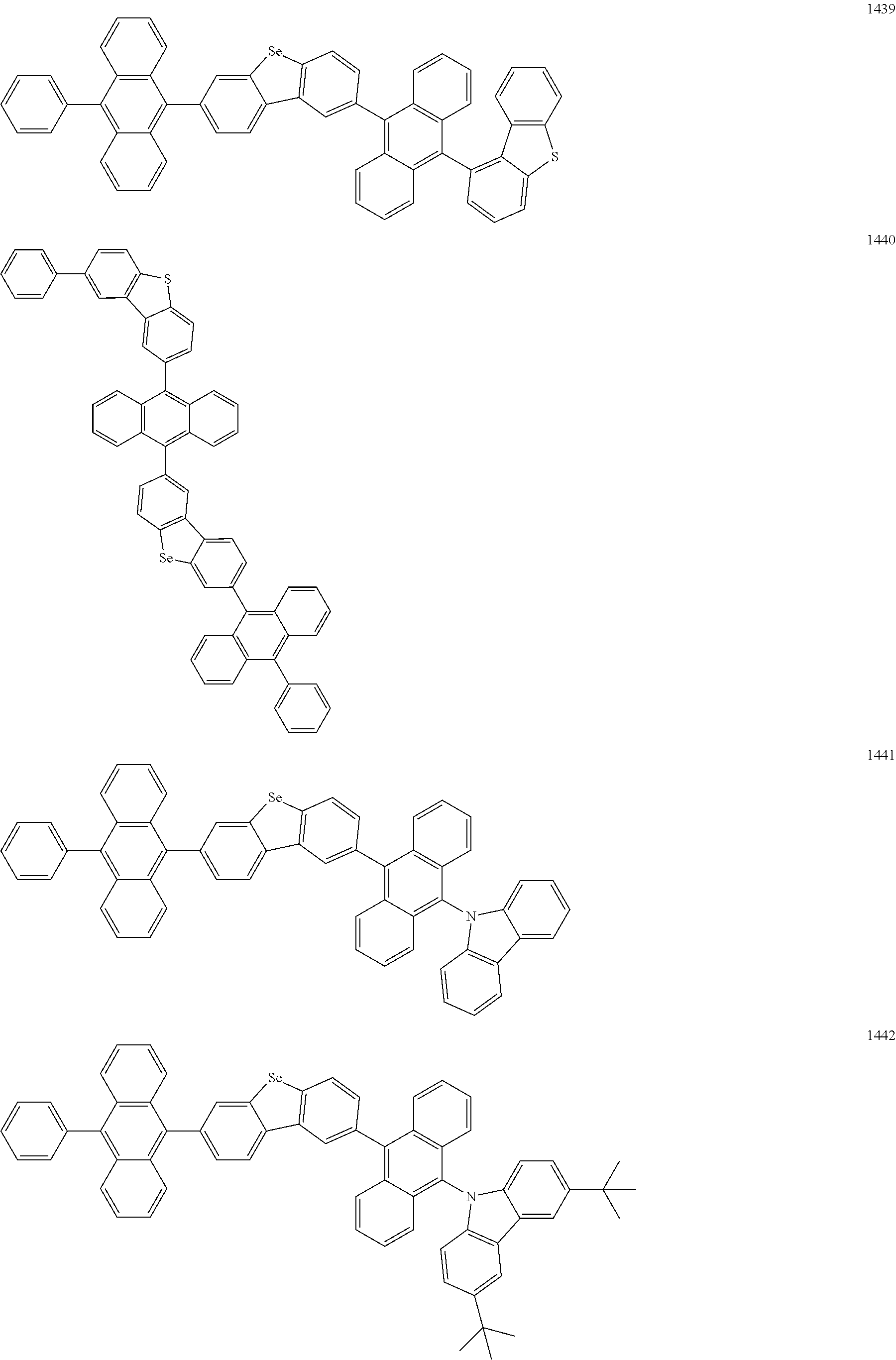 [SMILES: c1ccc(-c2c3ccccc3c(-c3ccc4c(c3)[se]c3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7[se]c8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)[se]c3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3[se]c4cc(-c5c6ccccc6c(-c6ccccc6)c6ccccc56)ccc4c3c2)c2ccccc12]

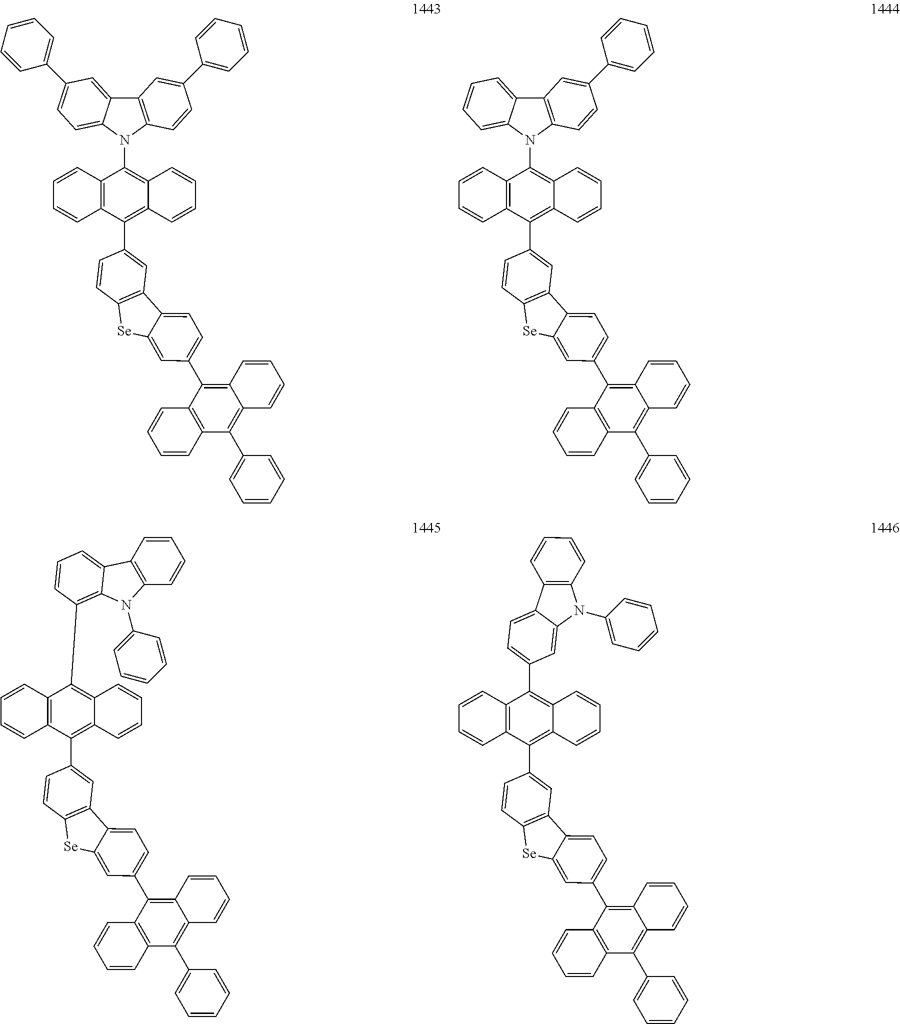 [SMILES: c1ccc(-c2ccc3c(c2)c2cc(-c4ccccc4)ccc2n3-c2c3ccccc3c(-c3ccc4[se]c5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4[se]c5cc(-c6c7ccccc7c(-c7ccccc7)c7ccccc67)ccc5c4c3)c3ccccc23)cc1 ; c1ccc(-c2c3ccccc3c(-c3ccc4c(c3)[se]c3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)[se]c3ccc(-c5c6ccccc6c(-c6ccc7c8ccccc8n(-c8ccccc8)c7c6)c6ccccc56)cc34)c3ccccc23)cc1]

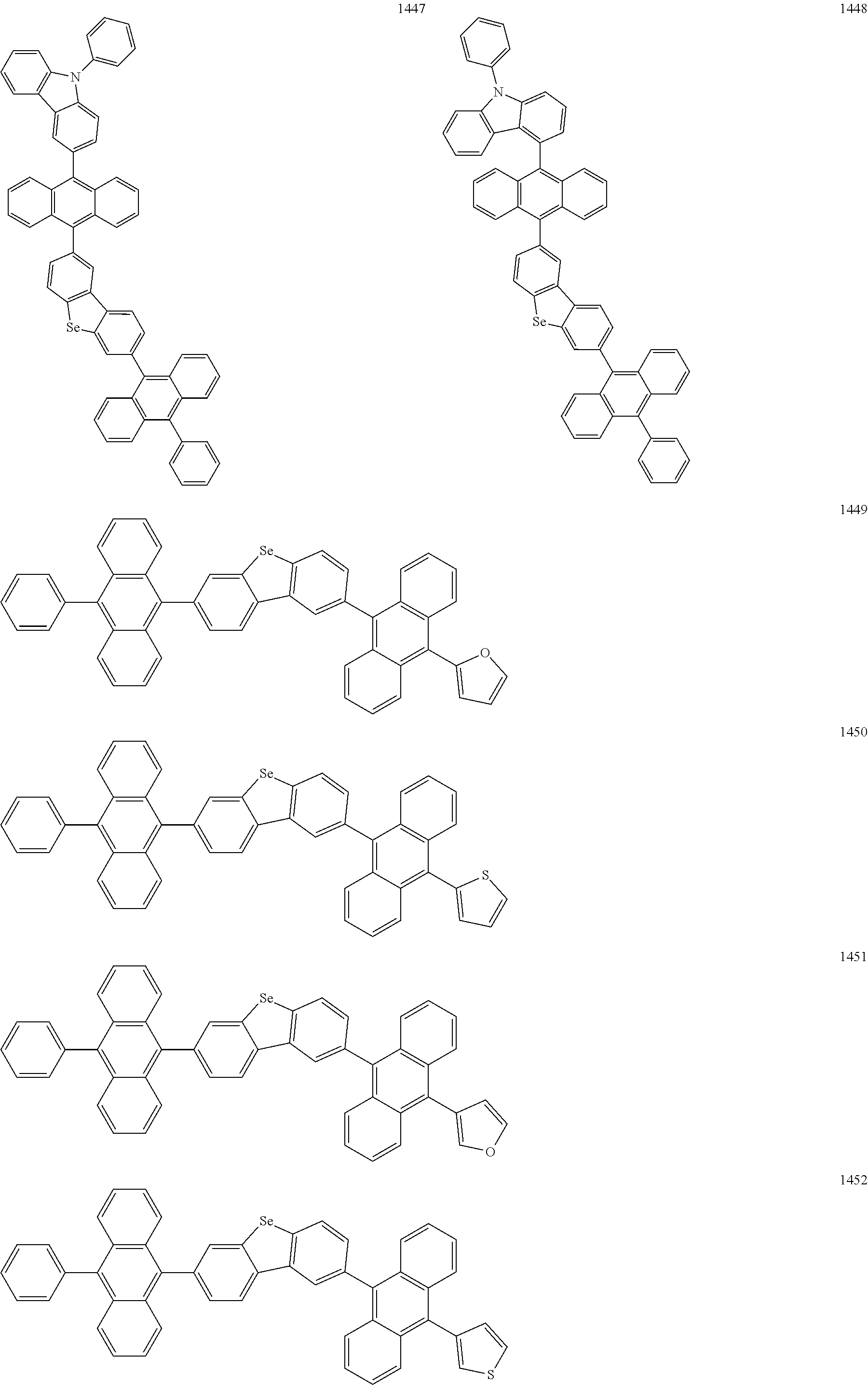

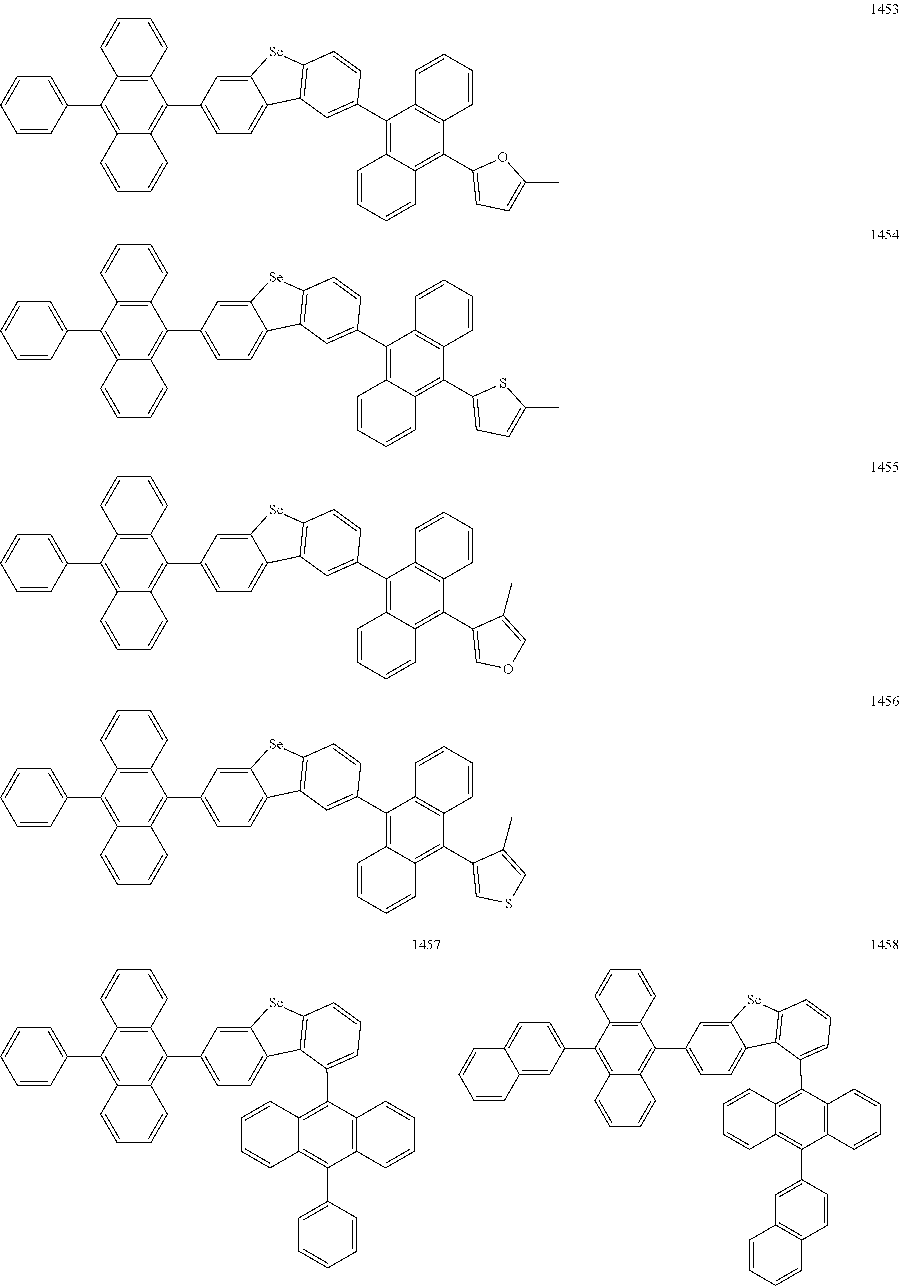

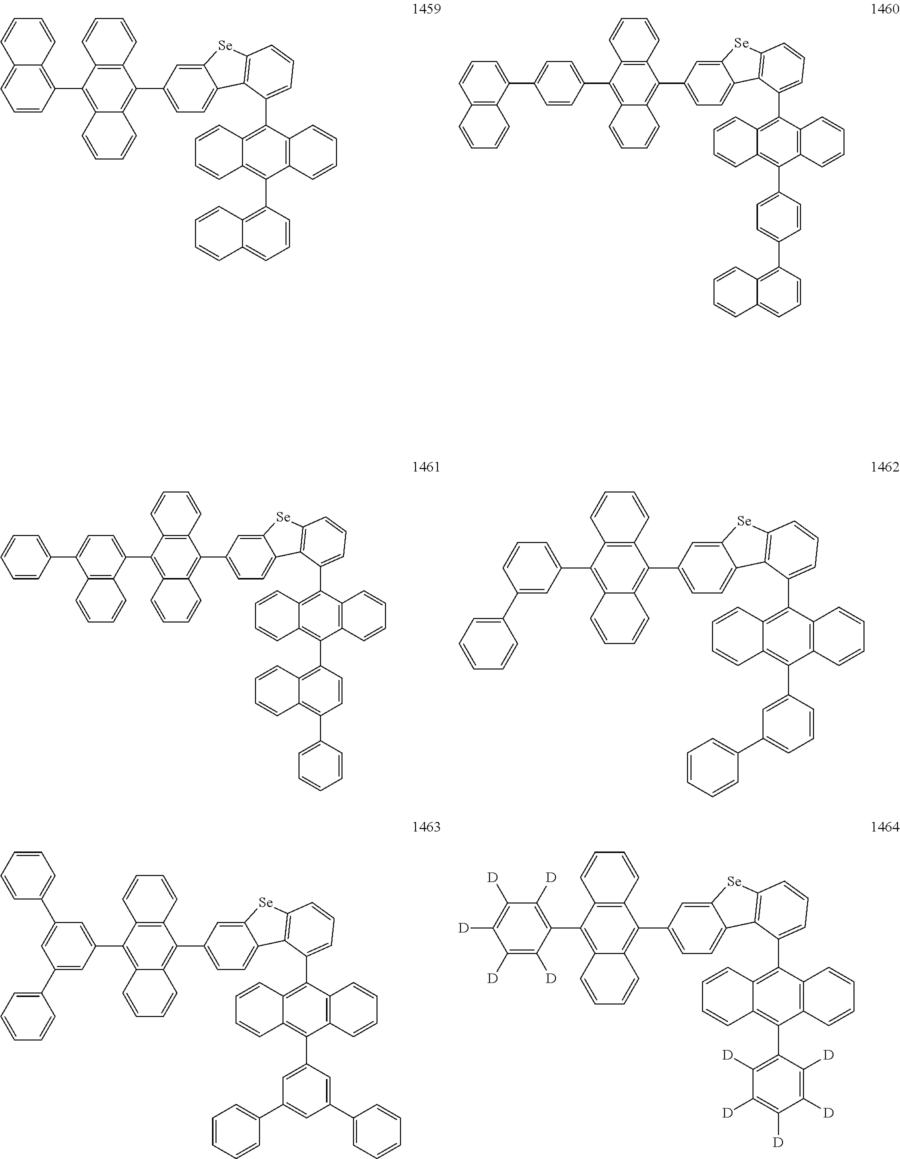

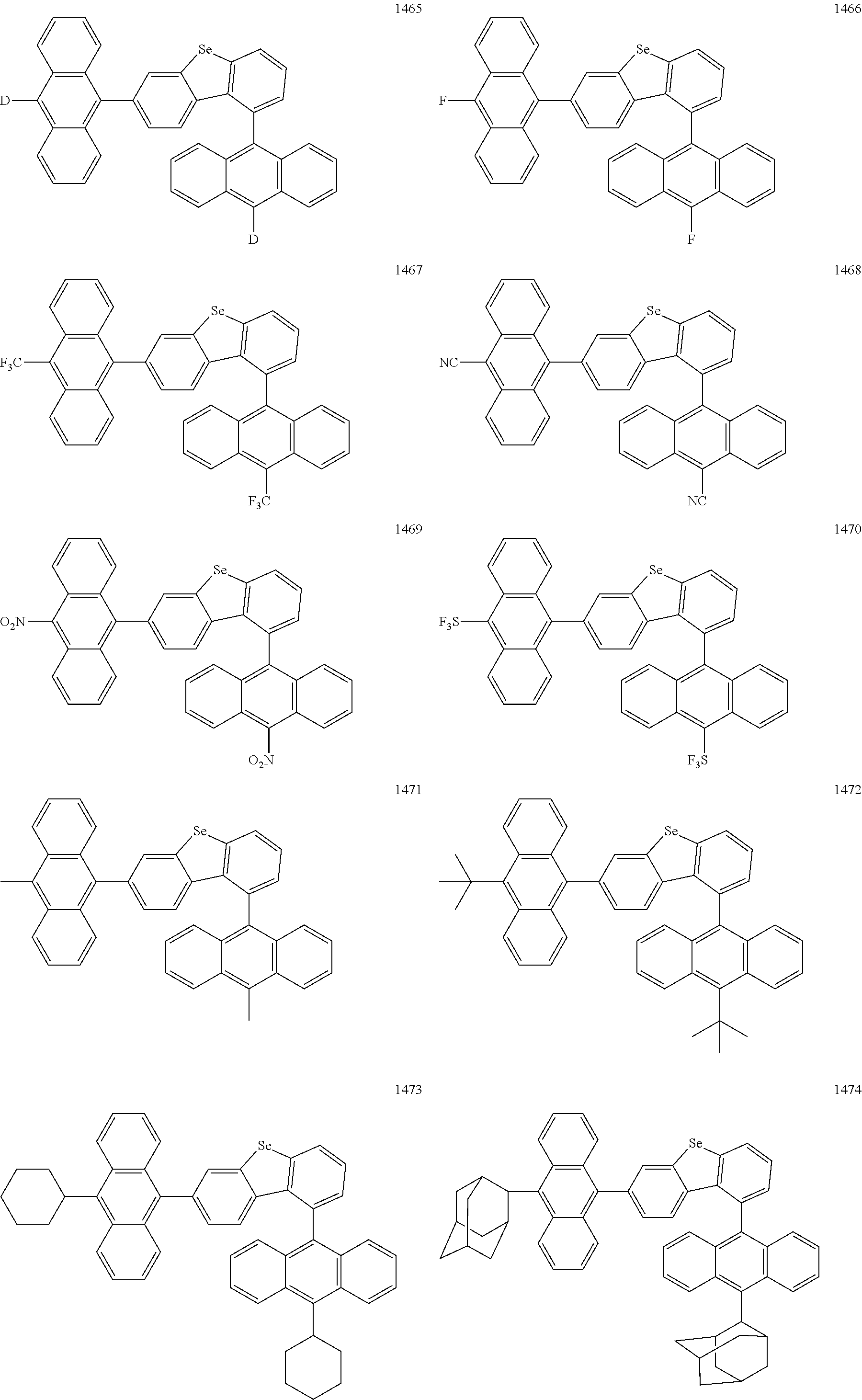

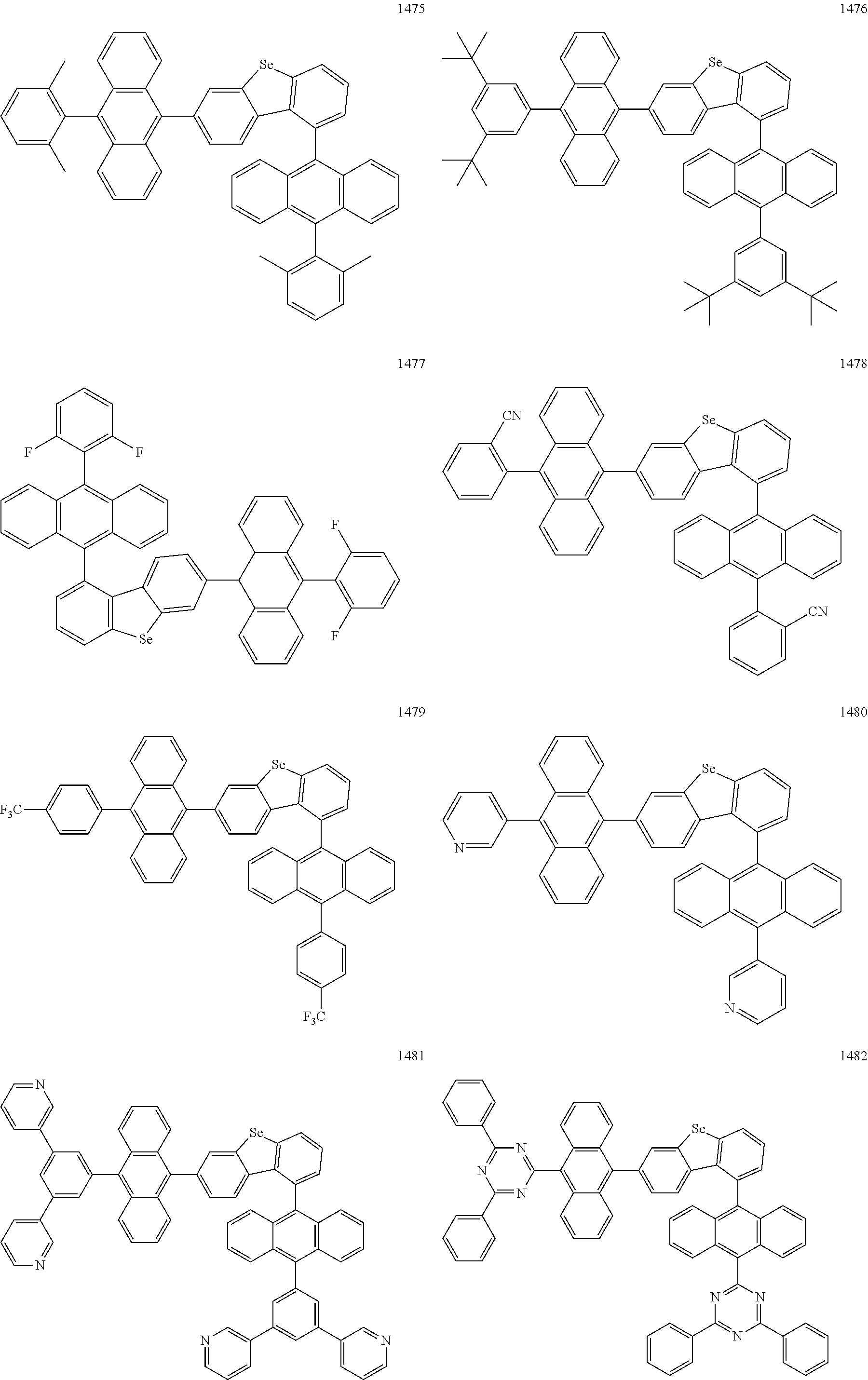

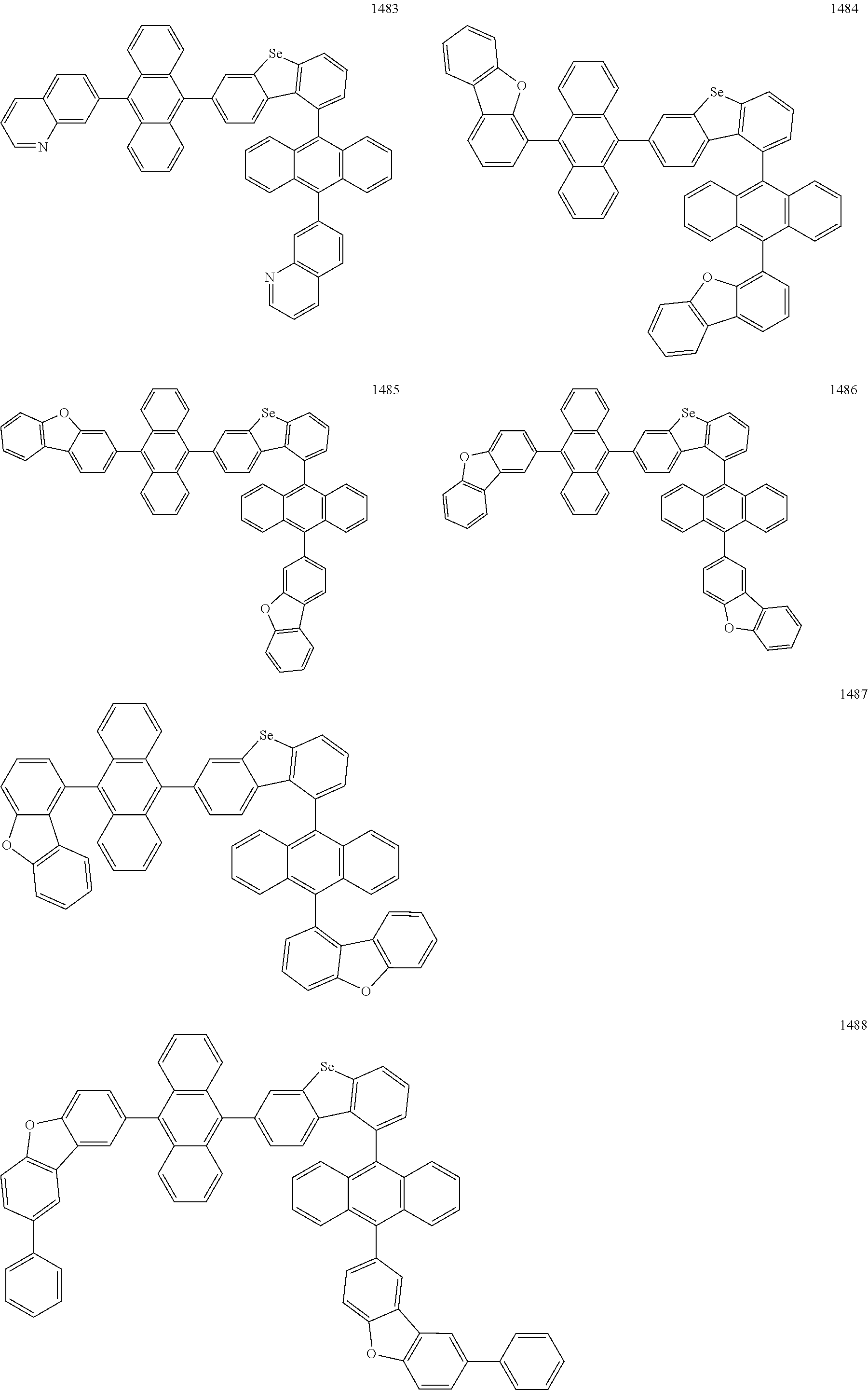

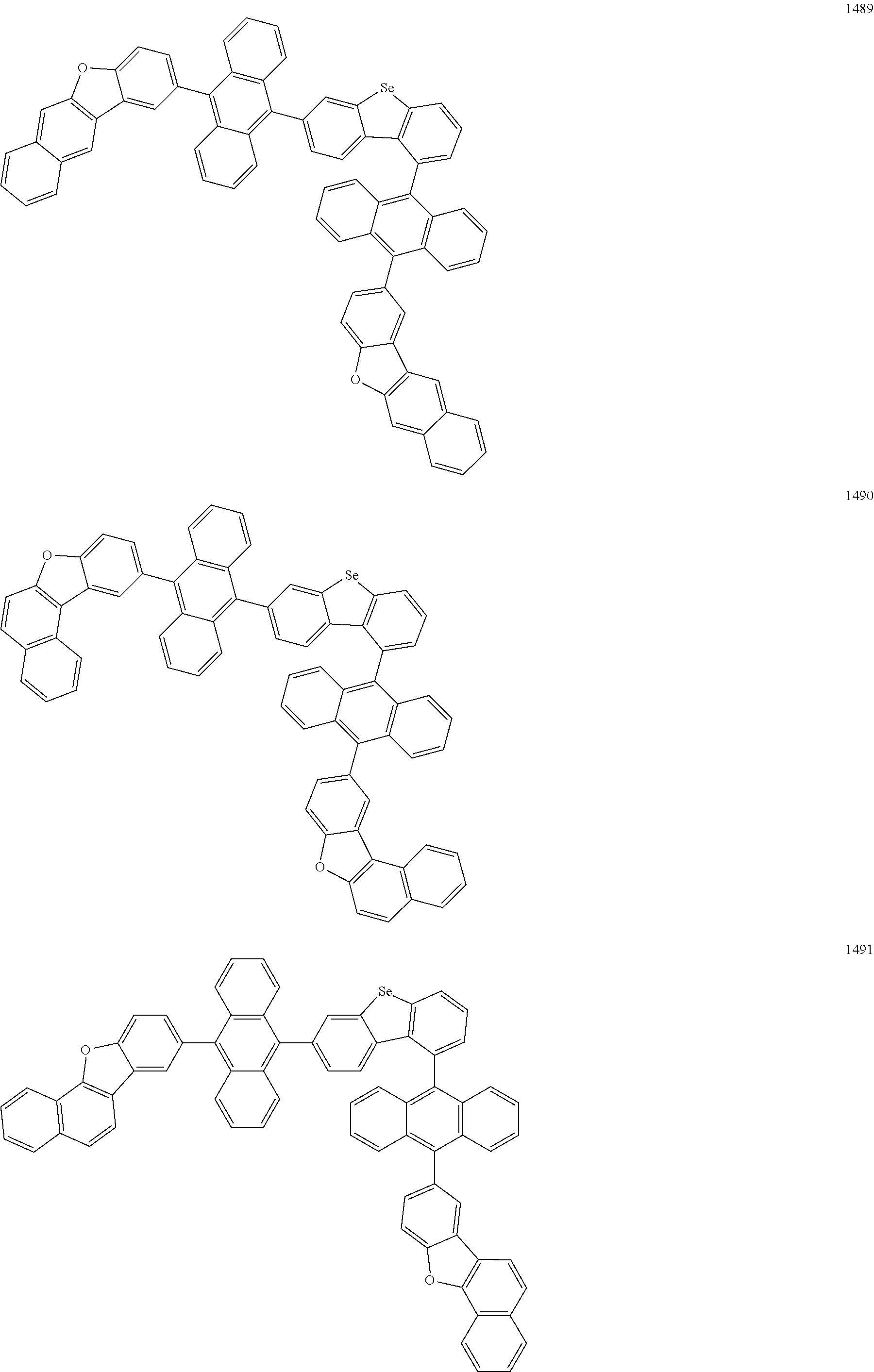 [SMILES: c1ccc2cc3c(cc2c1)oc1ccc(-c2c4ccccc4c(-c4ccc5c(c4)[se]c4cccc(-c6c7ccccc7c(-c7ccc8oc9cc%10ccccc%10cc9c8c7)c7ccccc67)c45)c4ccccc24)cc13 ; c1ccc2c(c1)ccc1oc3ccc(-c4c5ccccc5c(-c5ccc6c(c5)[se]c5cccc(-c7c8ccccc8c(-c8ccc9oc%10ccc%11ccccc%11c%10c9c8)c8ccccc78)c56)c5ccccc45)cc3c12 ; c1ccc2c(c1)ccc1c3cc(-c4c5ccccc5c(-c5ccc6c(c5)[se]c5cccc(-c7c8ccccc8c(-c8ccc9oc%10c%11ccccc%11ccc%10c9c8)c8ccccc78)c56)c5ccccc45)ccc3oc21]

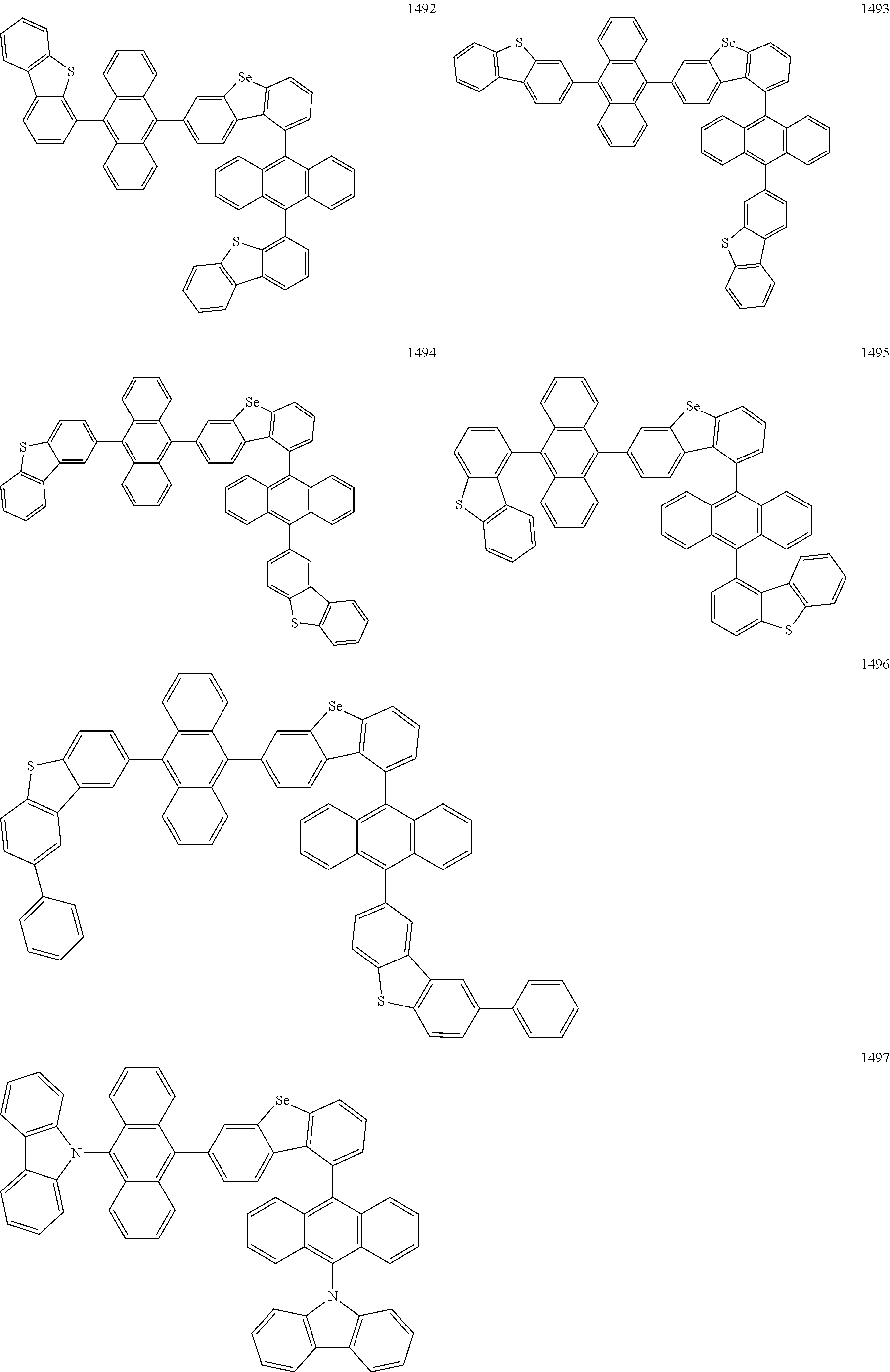

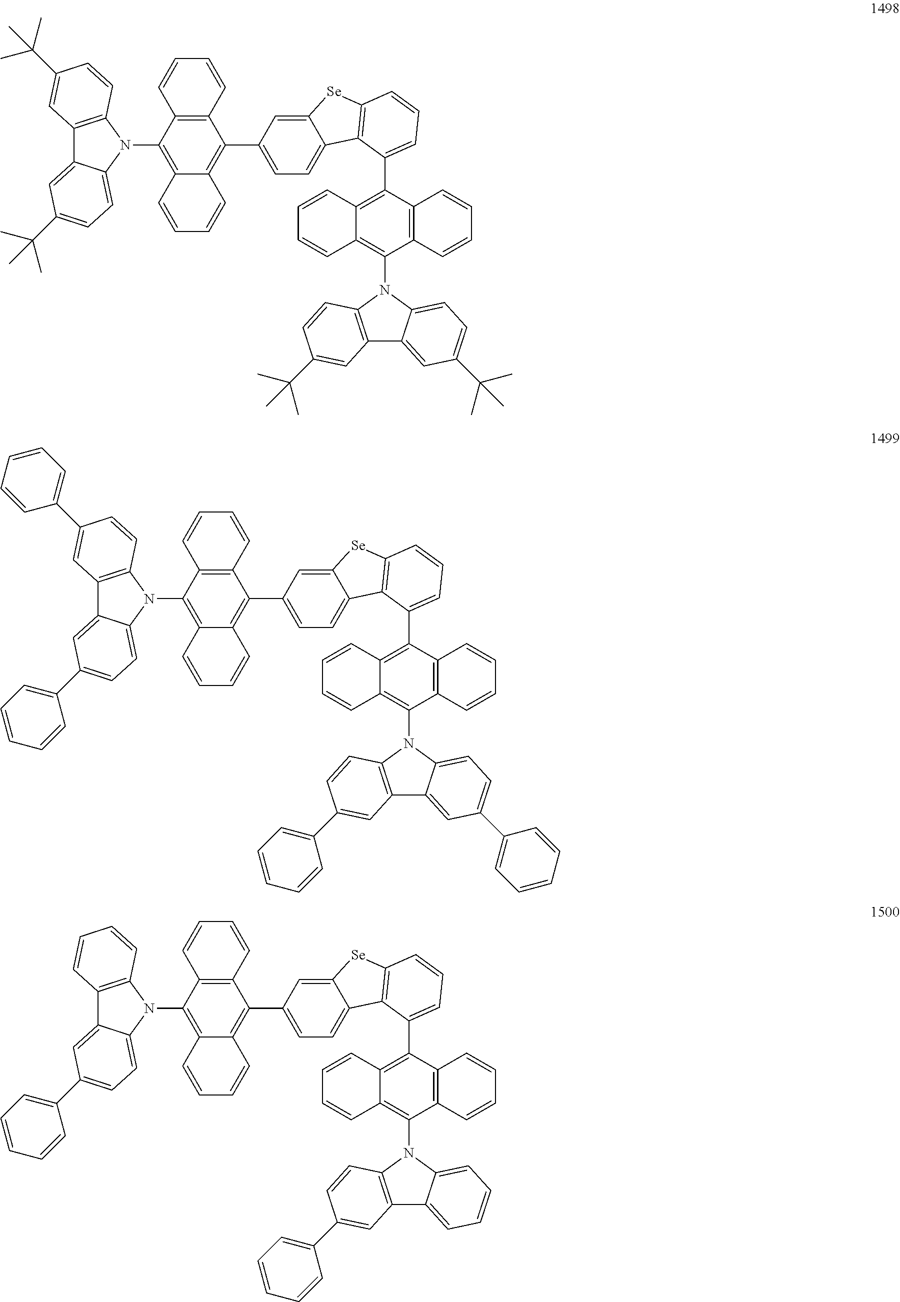 [SMILES: CC(C)(C)c1ccc2c(c1)c1cc(C(C)(C)C)ccc1n2-c1c2ccccc2c(-c2ccc3c(c2)[se]c2cccc(-c4c5ccccc5c(-n5c6ccc(C(C)(C)C)cc6c6cc(C(C)(C)C)ccc65)c5ccccc45)c23)c2ccccc12 ; c1ccc(-c2ccc3c(c2)c2cc(-c4ccccc4)ccc2n3-c2c3ccccc3c(-c3ccc4c(c3)[se]c3cccc(-c5c6ccccc6c(-n6c7ccc(-c8ccccc8)cc7c7cc(-c8ccccc8)ccc76)c6ccccc56)c34)c3ccccc23)cc1 ; c1ccc(-c2ccc3c(c2)c2ccccc2n3-c2c3ccccc3c(-c3ccc4c(c3)[se]c3cccc(-c5c6ccccc6c(-n6c7ccccc7c7cc(-c8ccccc8)ccc76)c6ccccc56)c34)c3ccccc23)cc1]

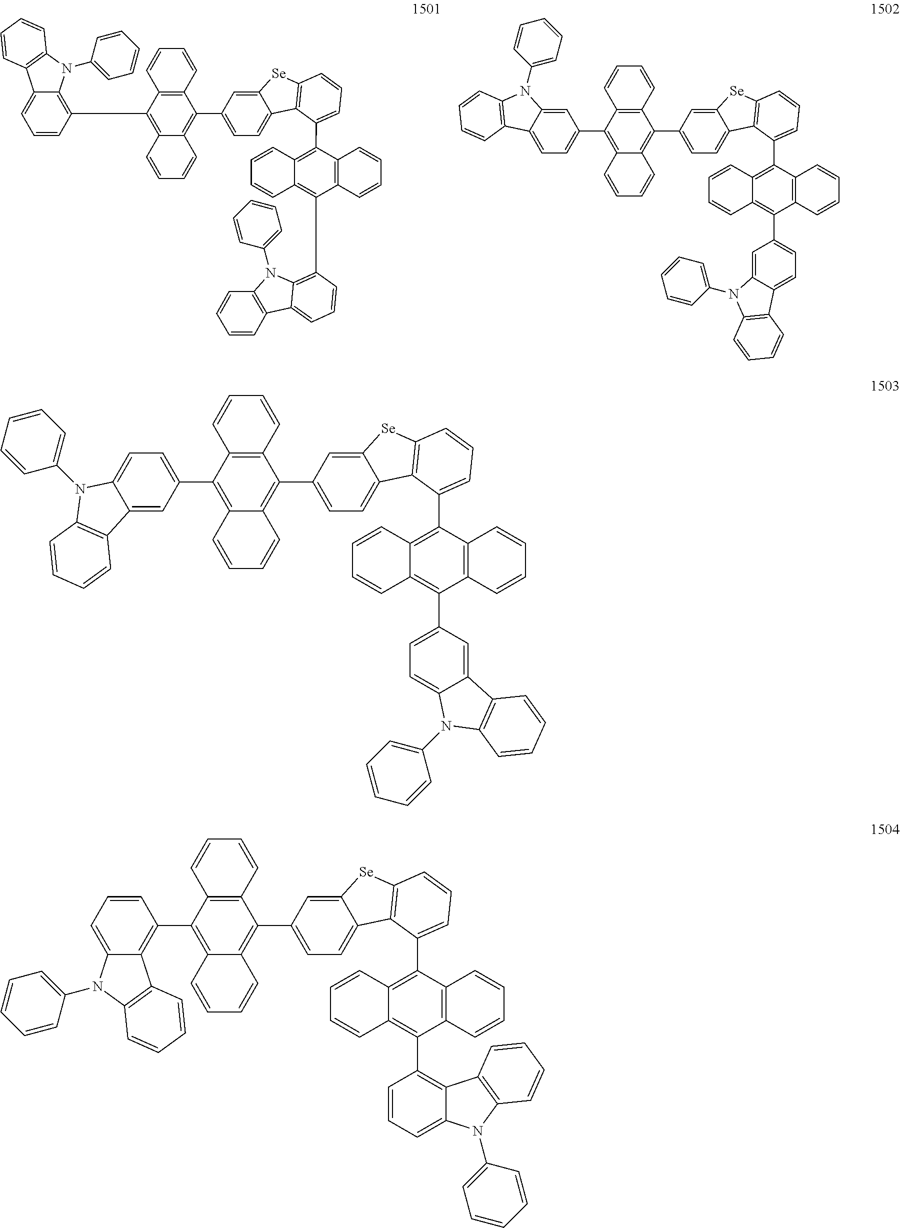

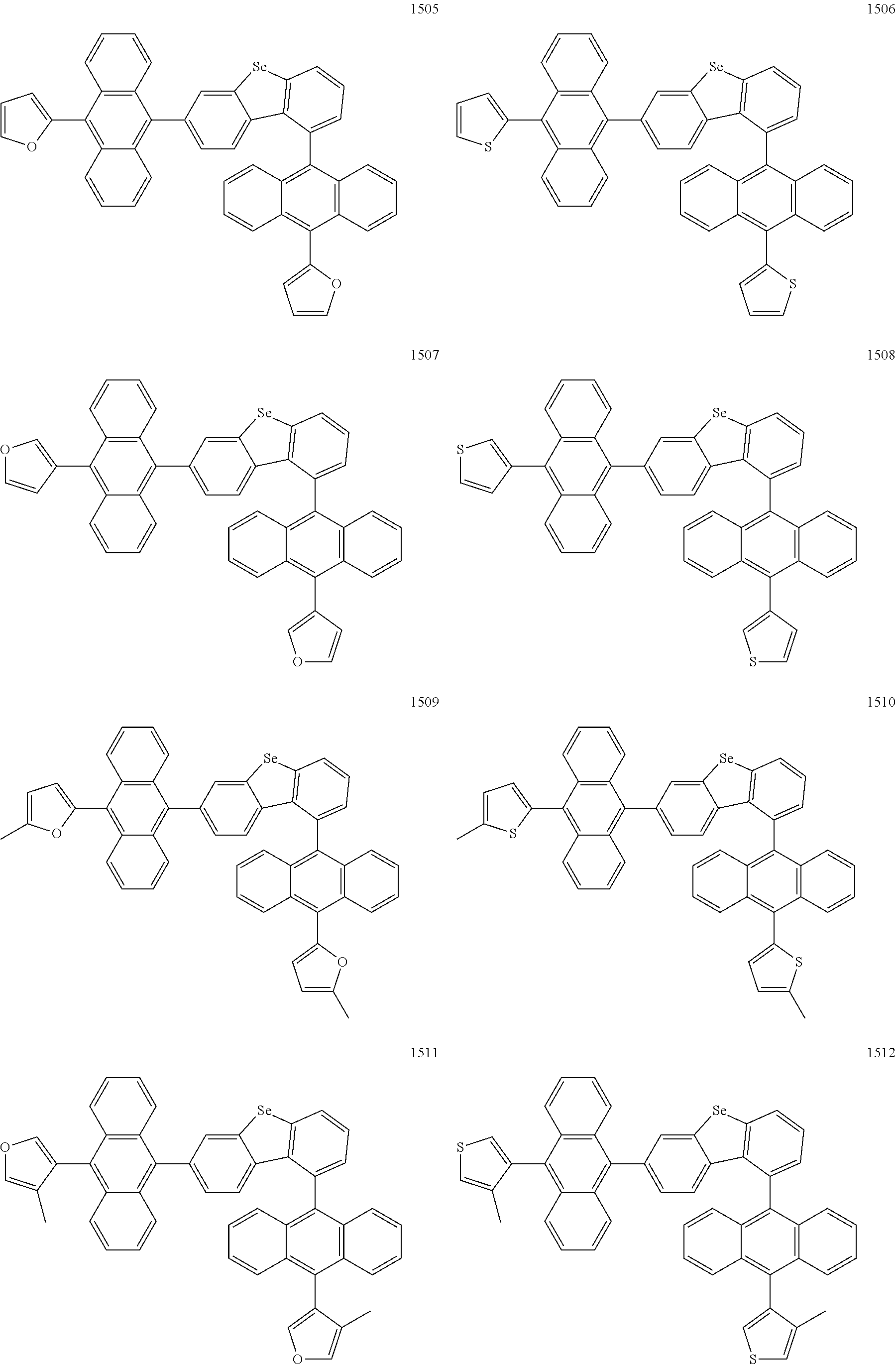

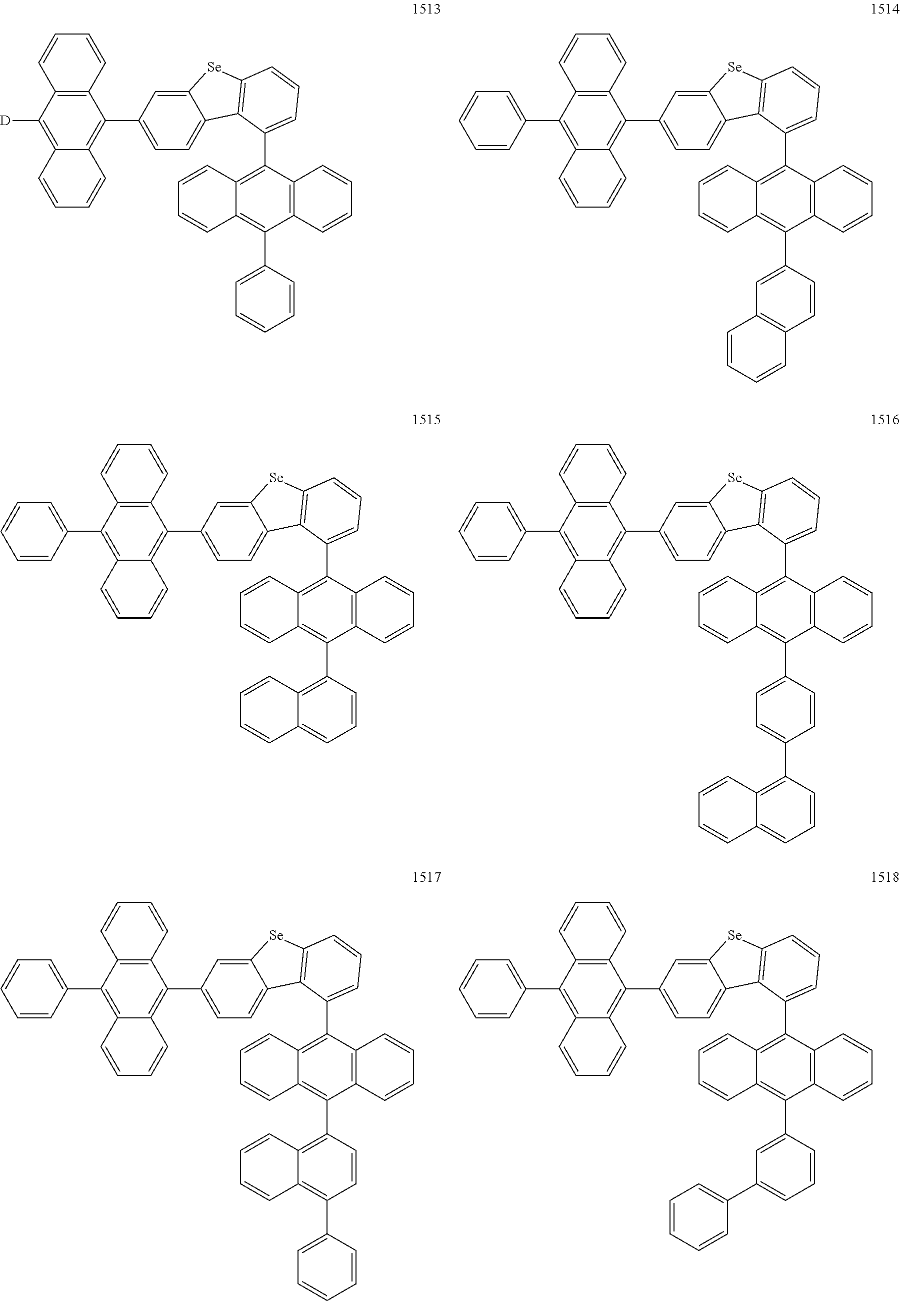

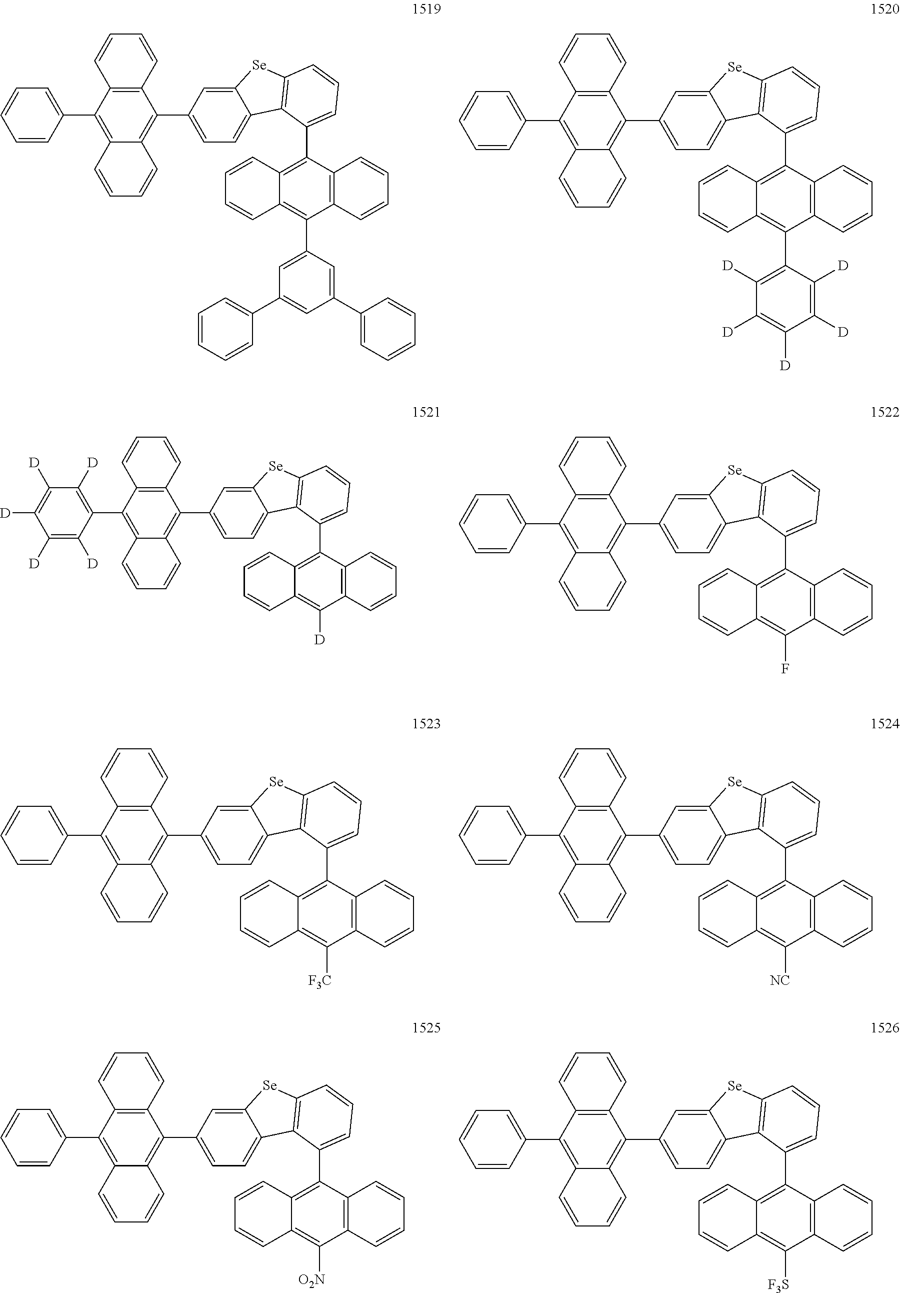

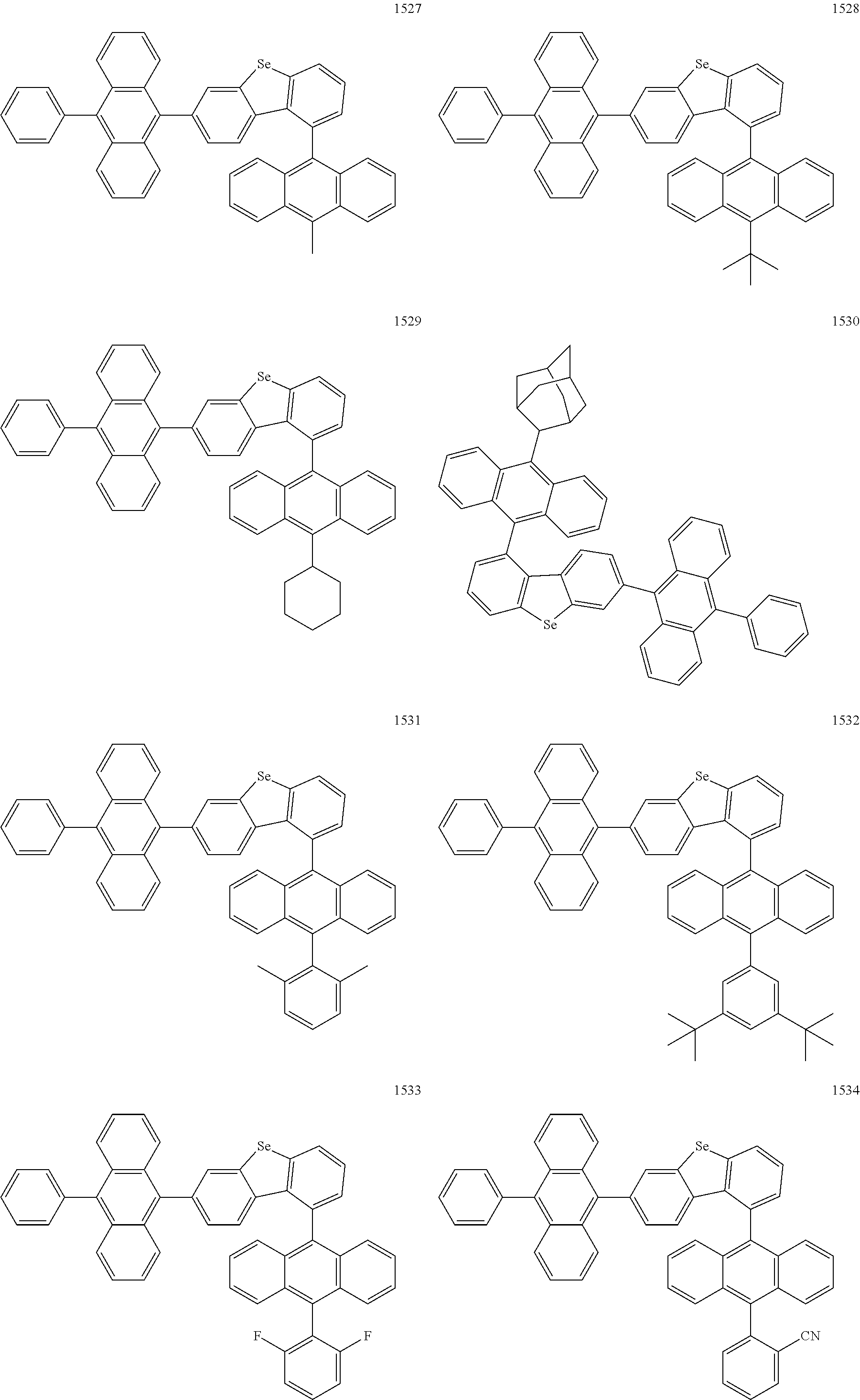

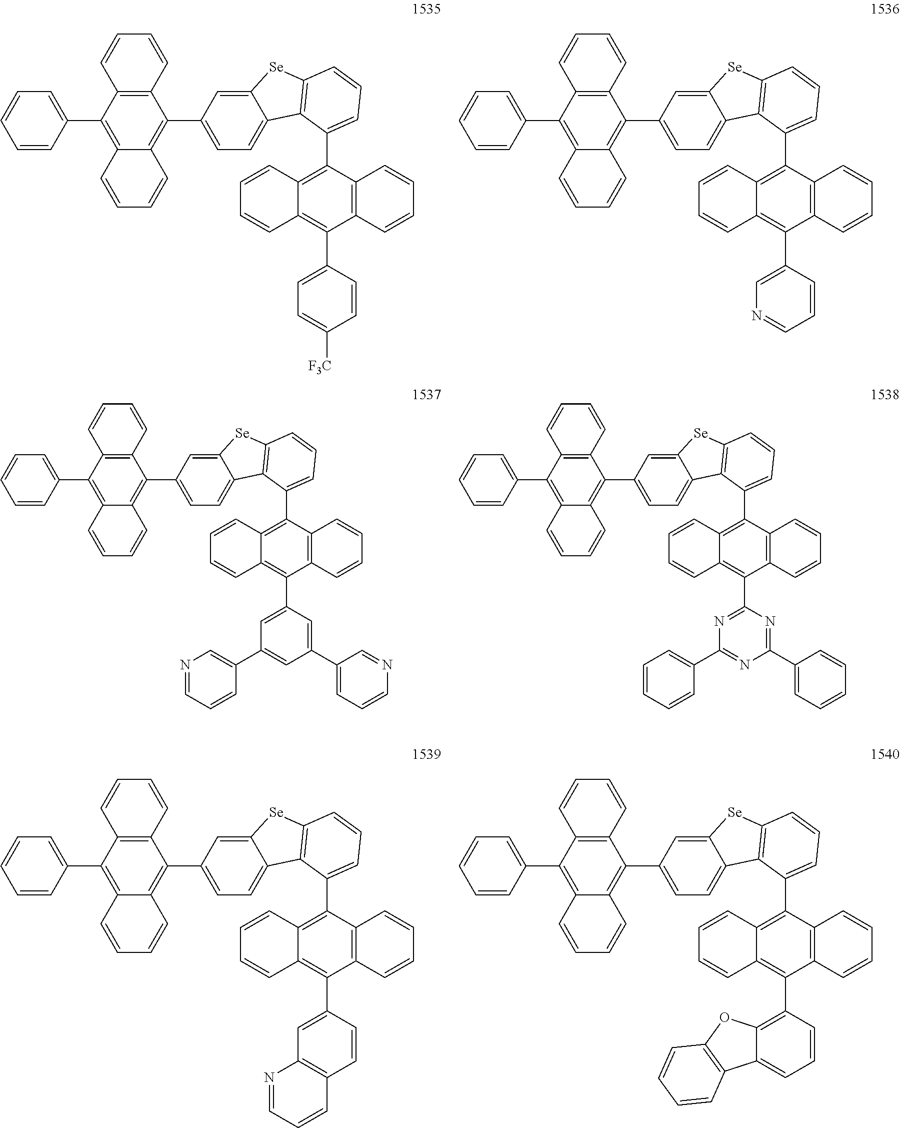

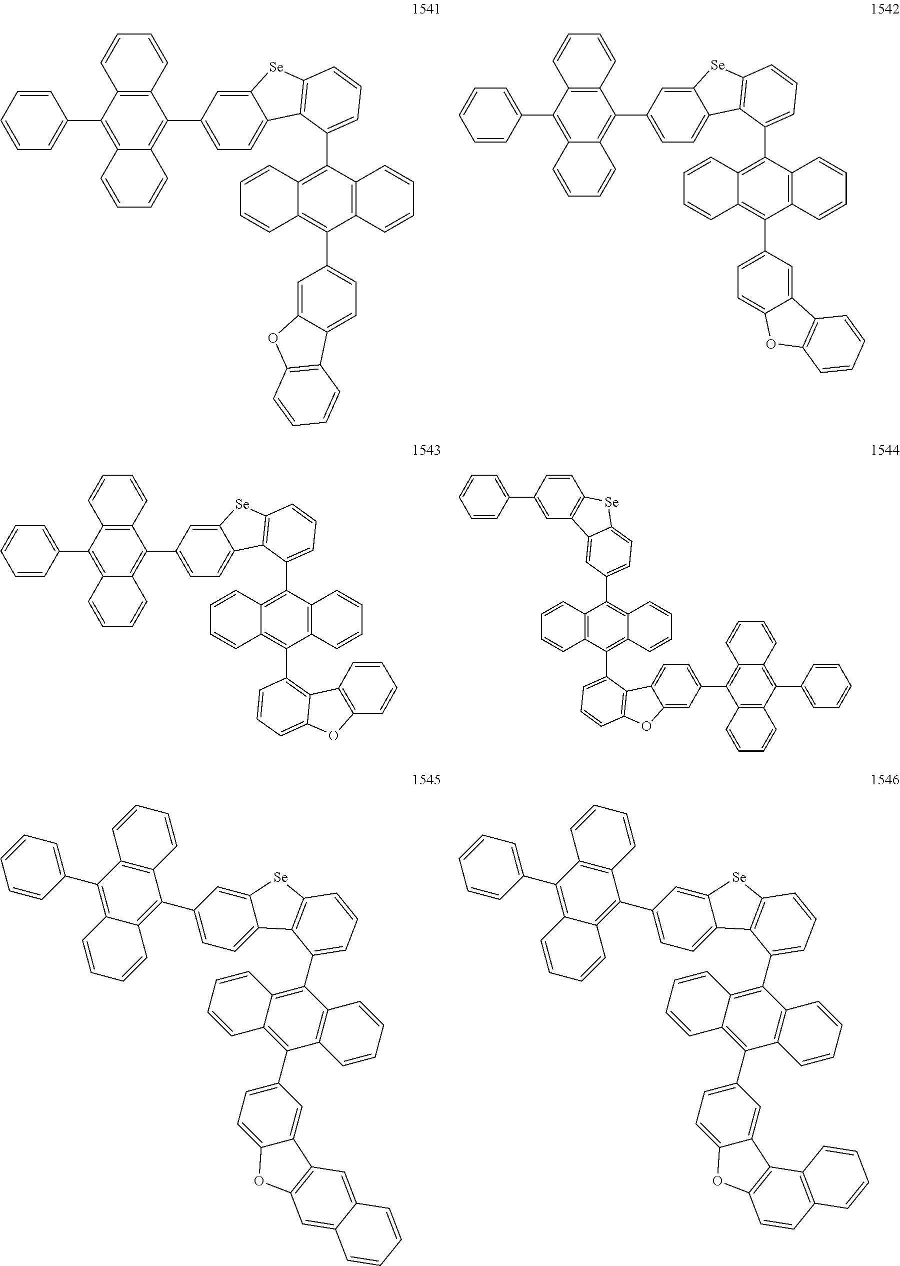

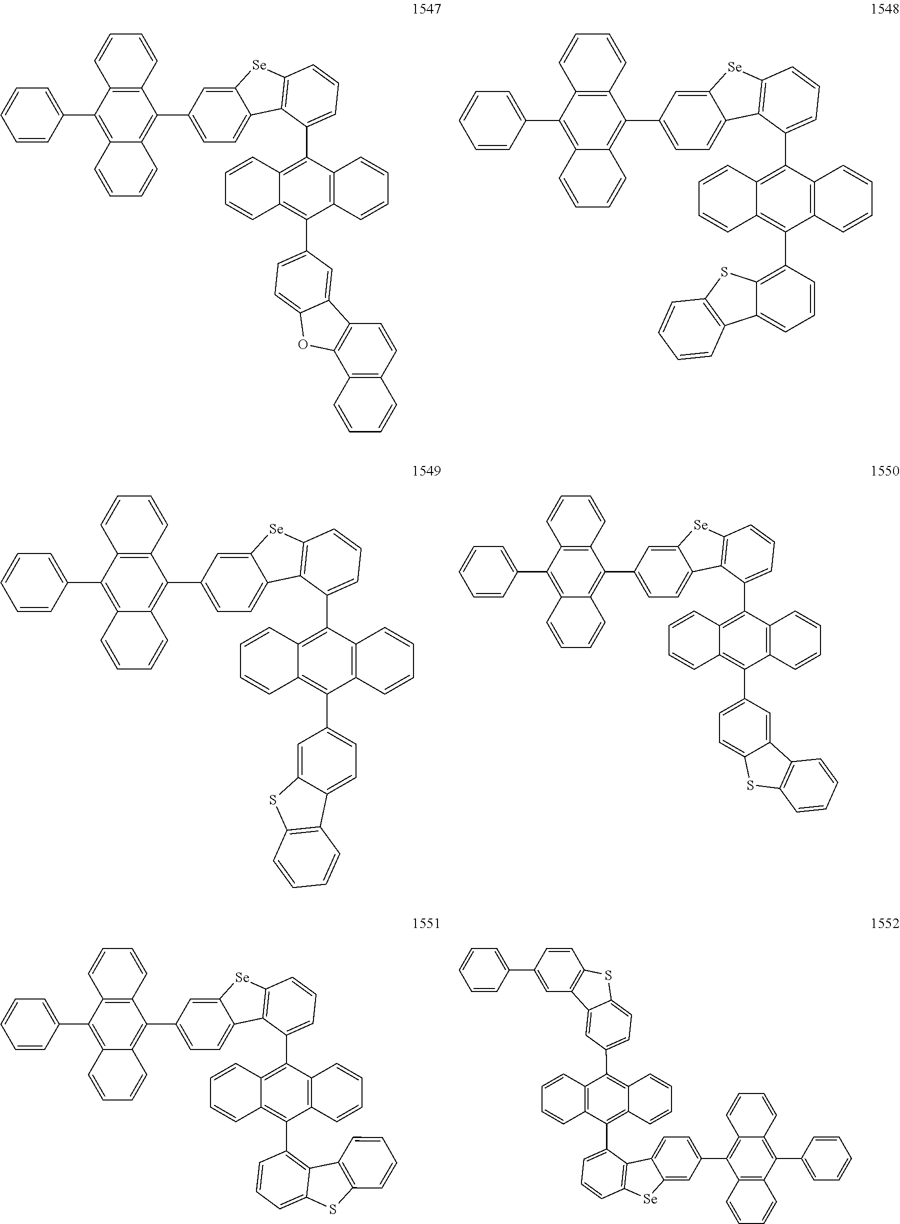

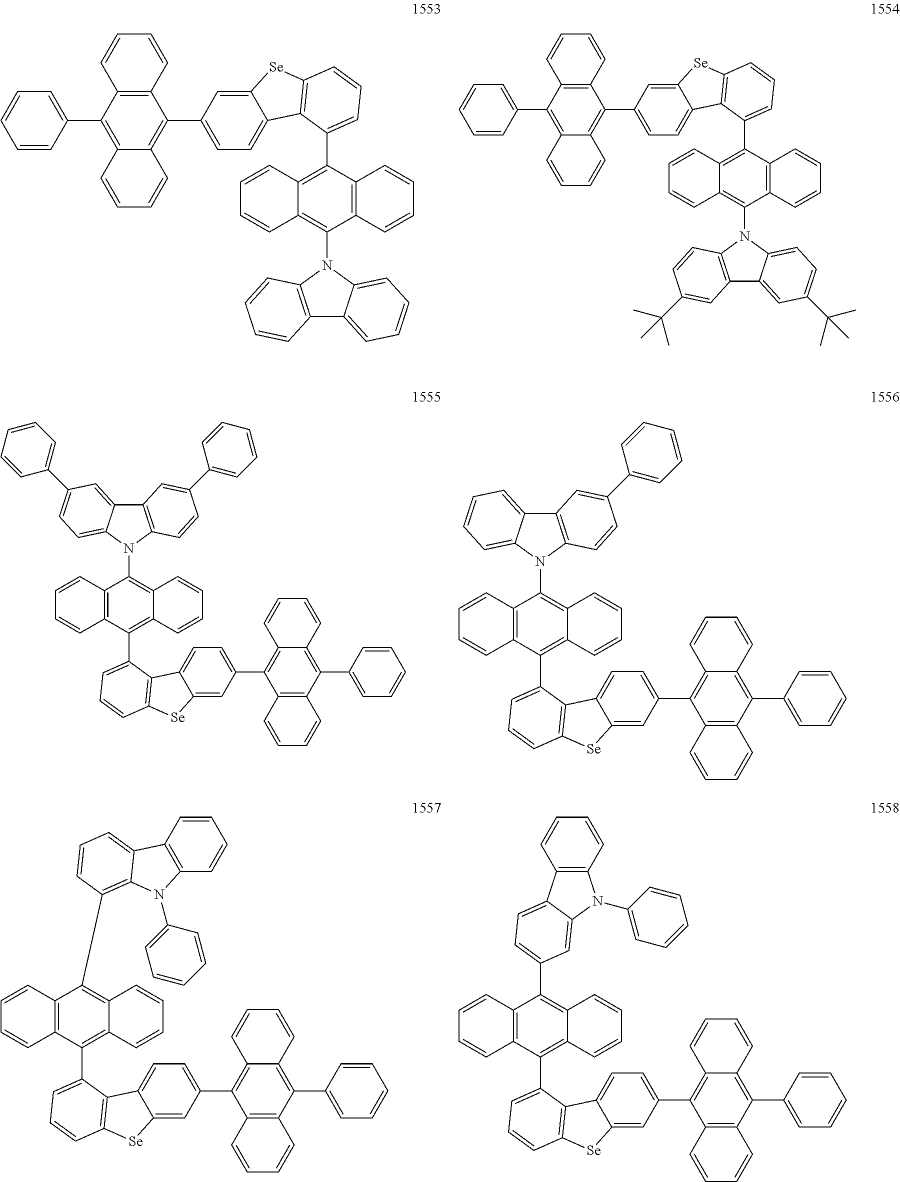

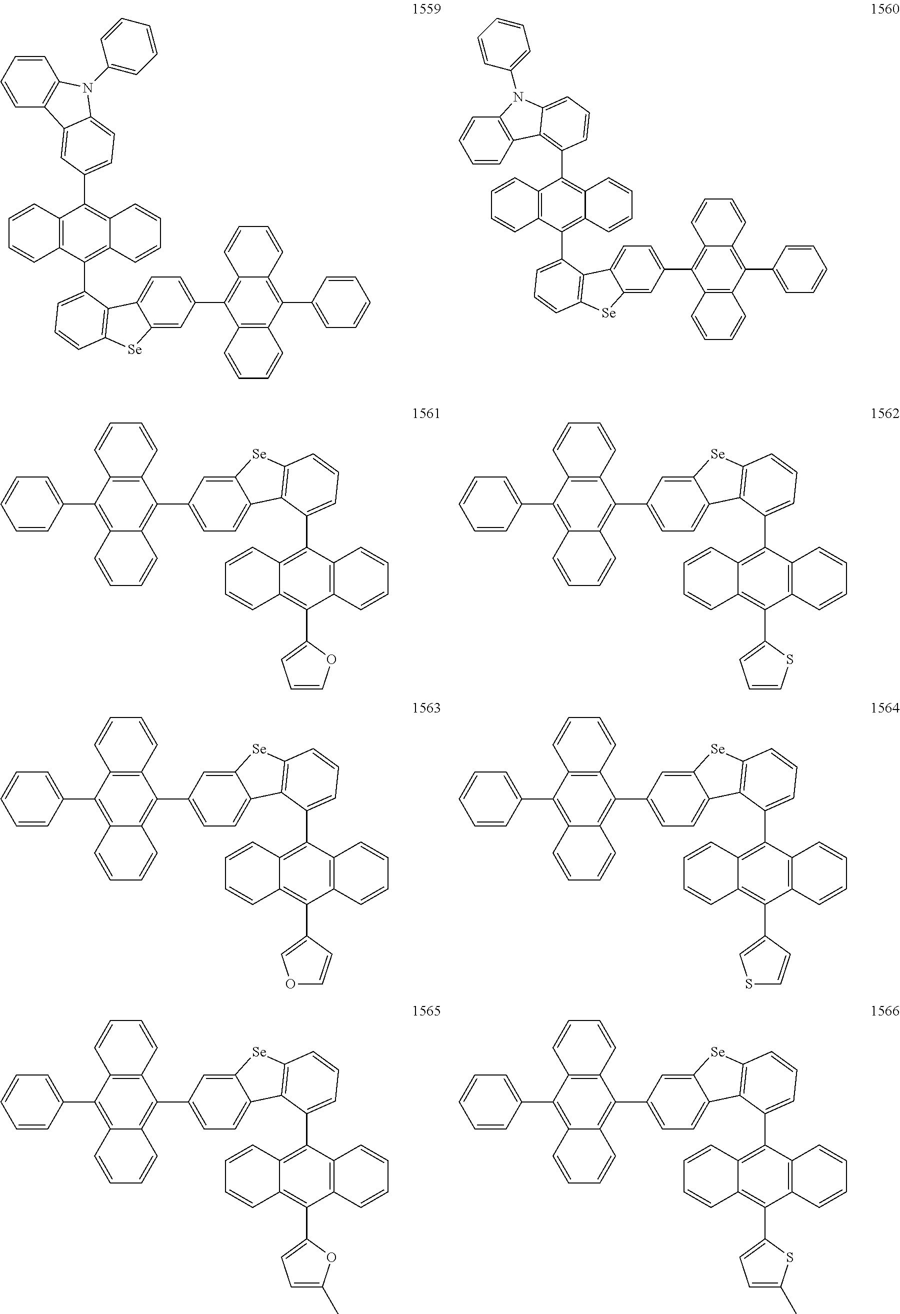

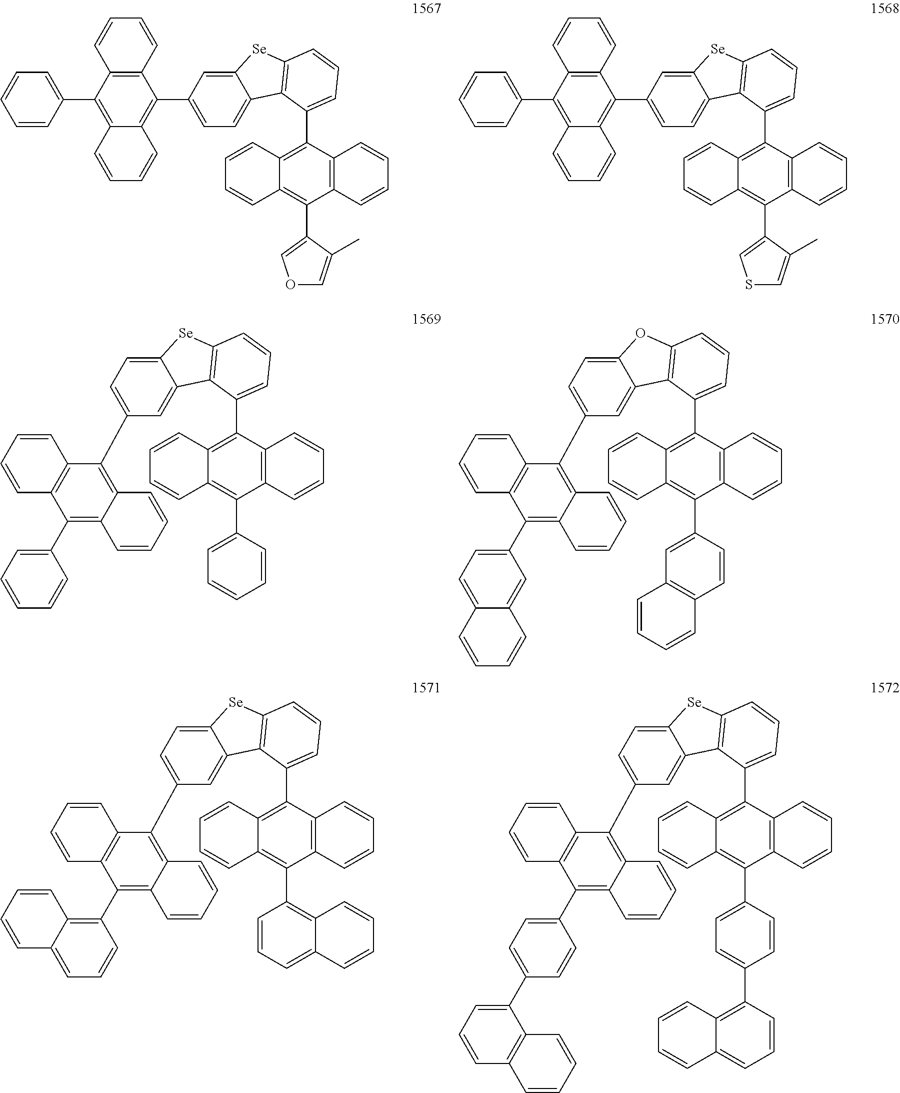

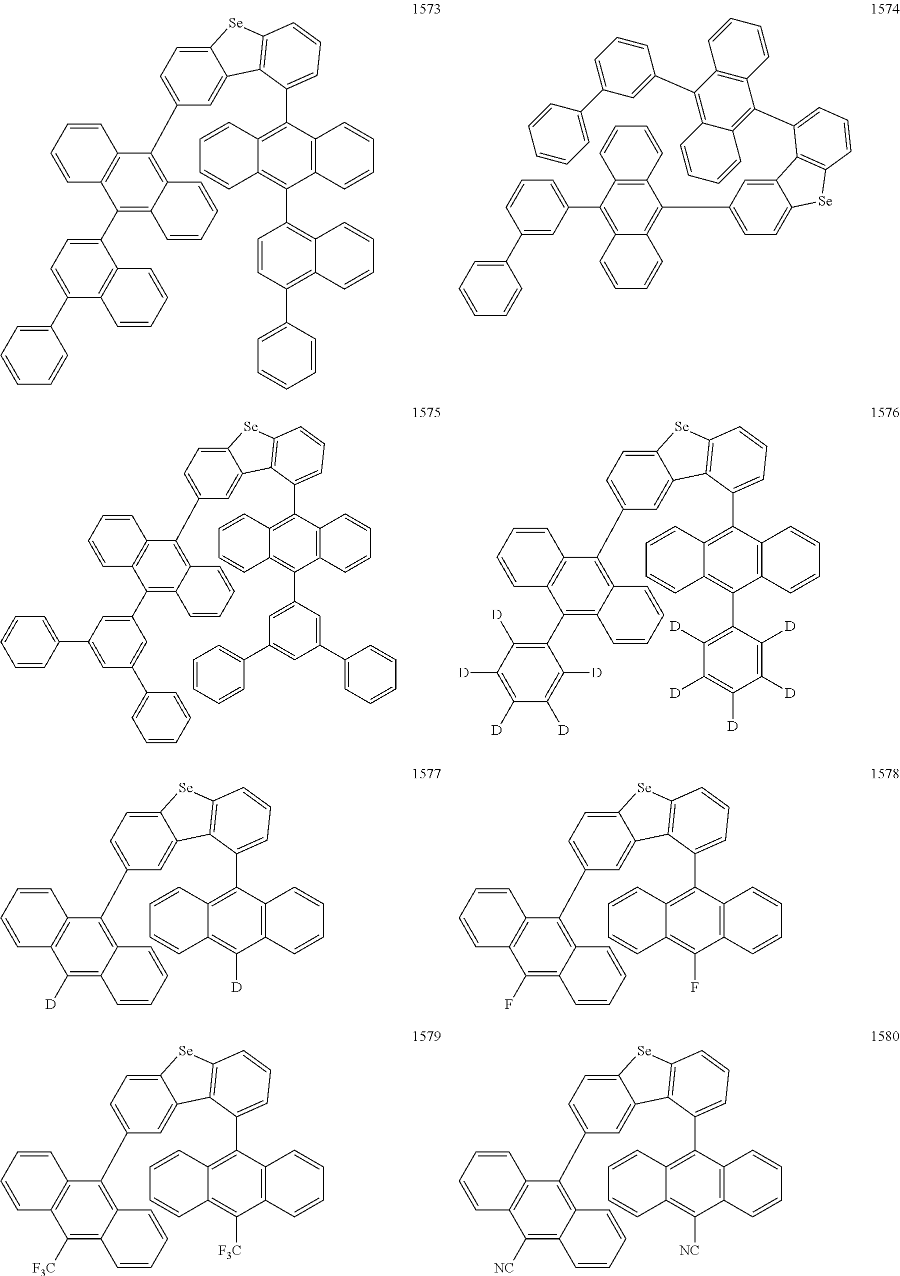

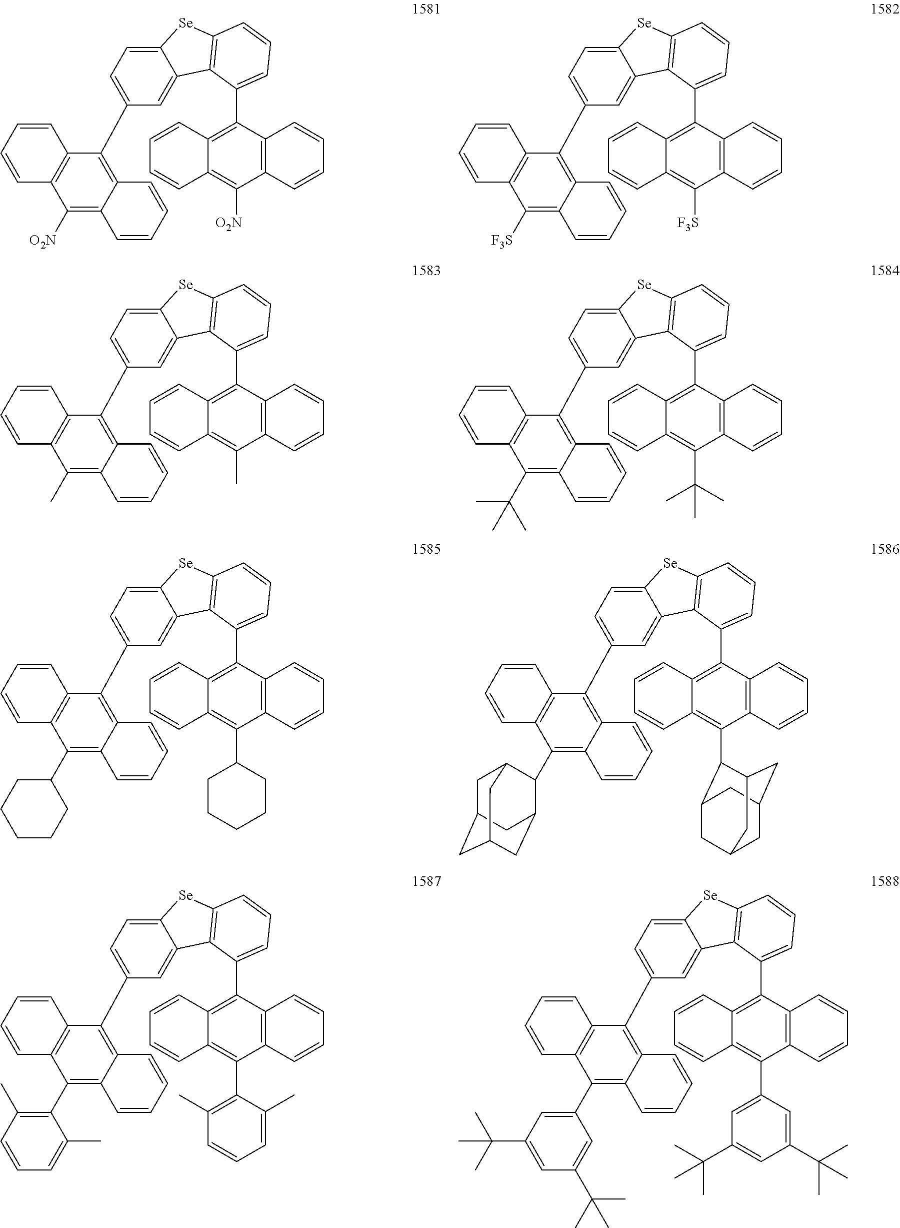

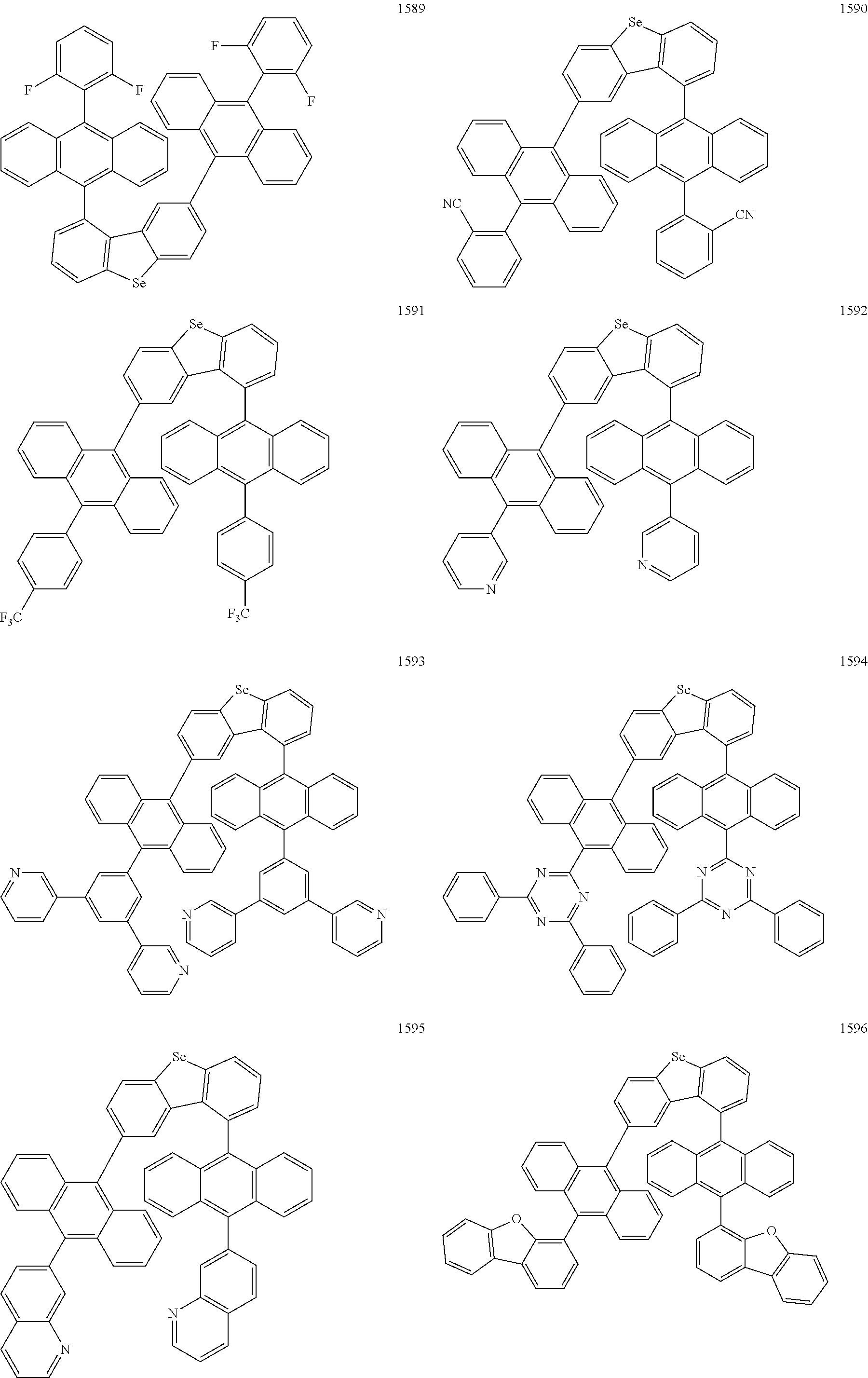

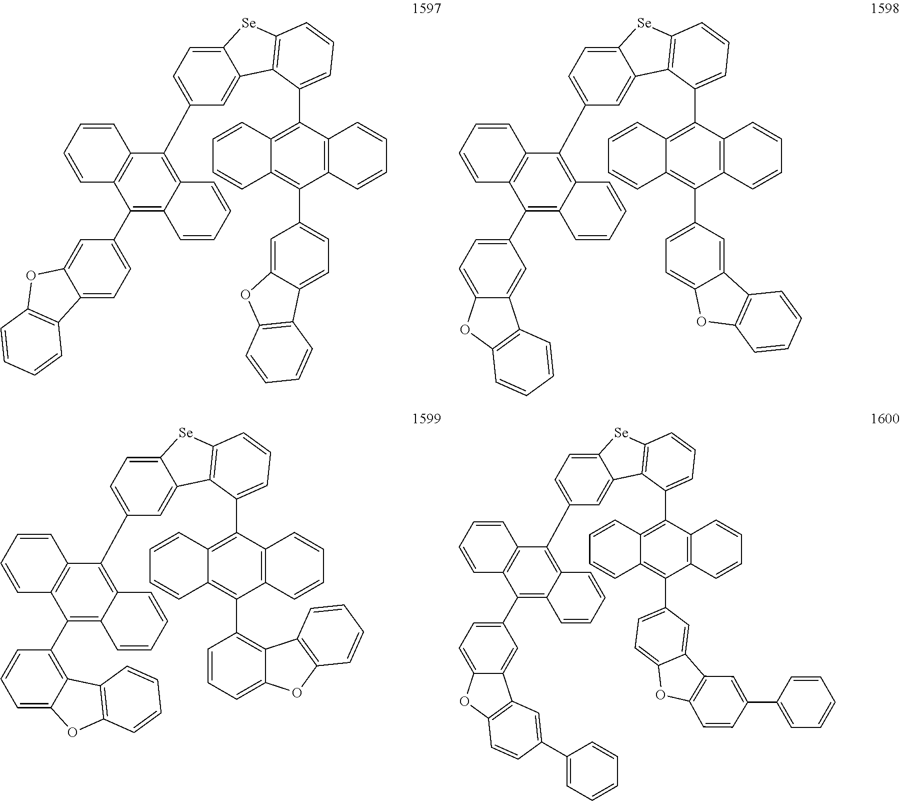

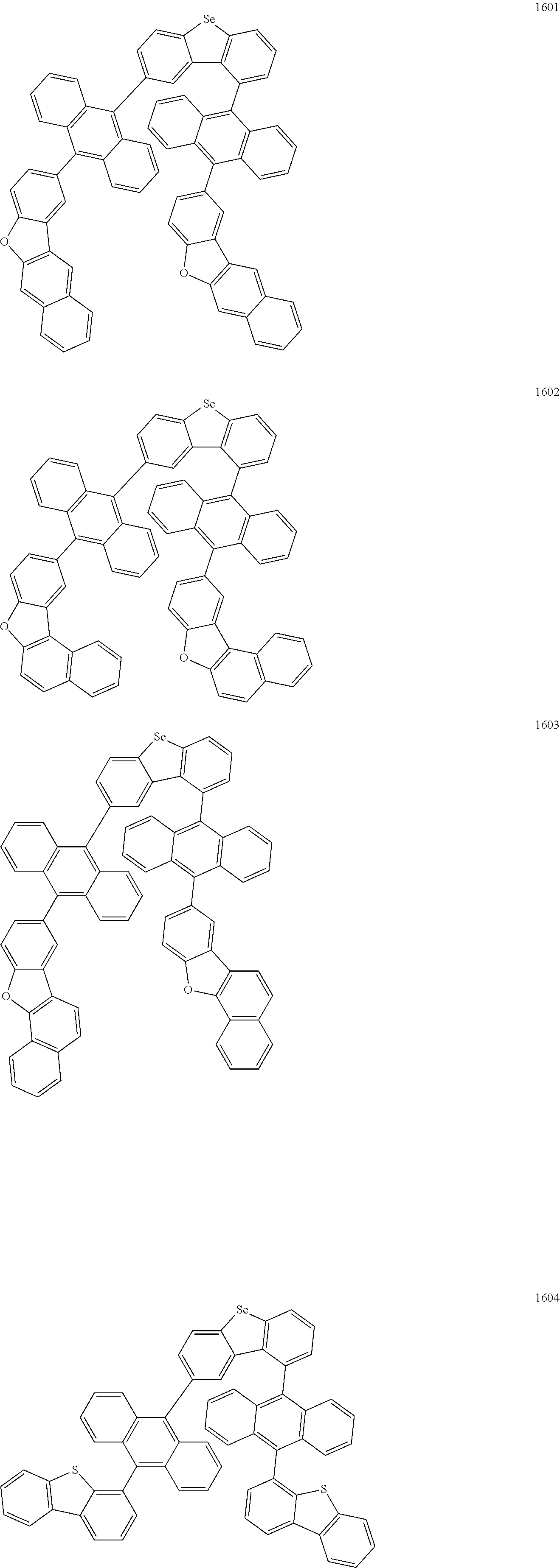

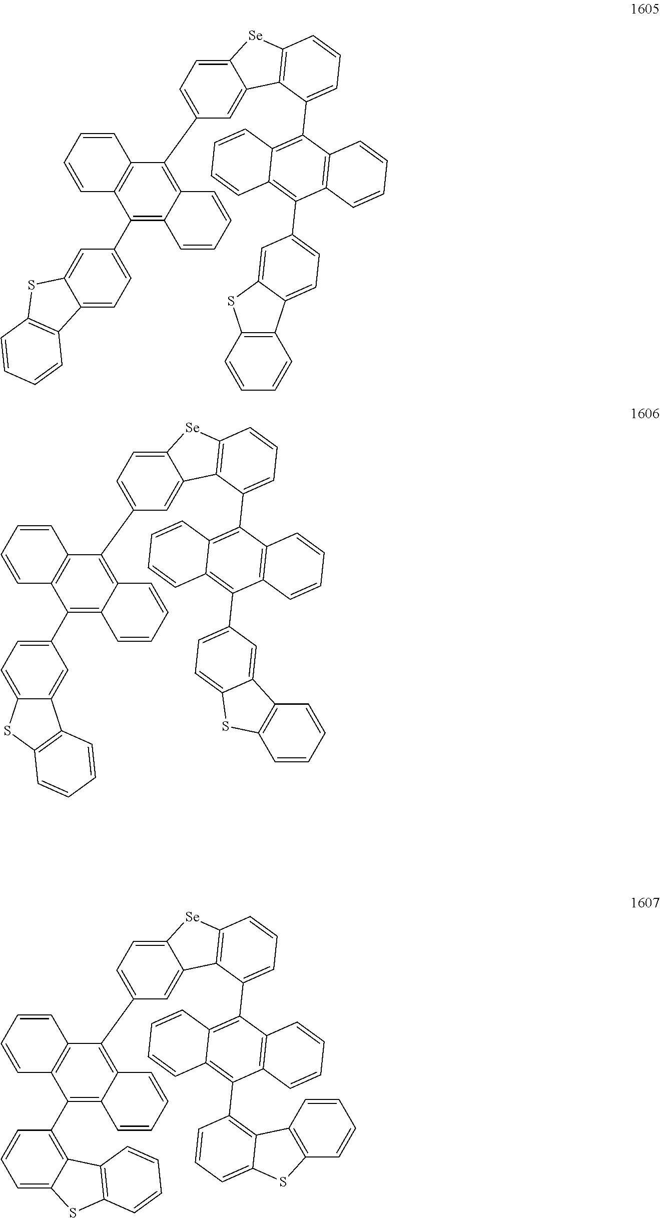

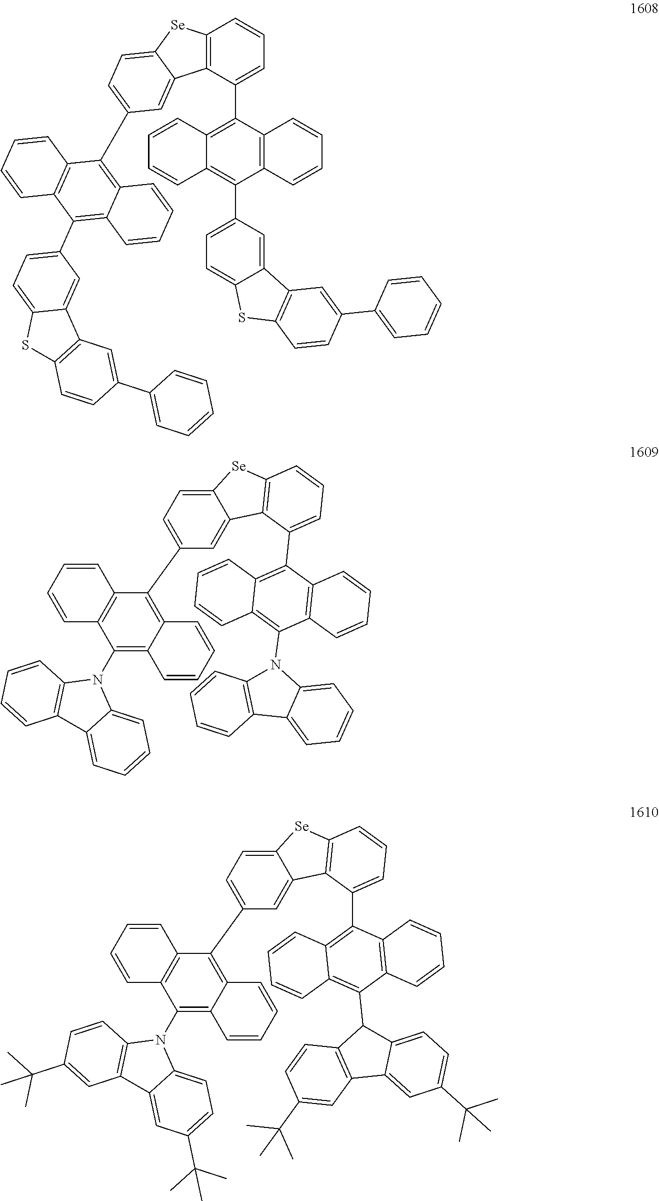 [SMILES: c1ccc(-c2ccc3sc4ccc(-c5c6ccccc6c(-c6ccc7[se]c8cccc(-c9c%10ccccc%10c(-c%10ccc%11sc%12ccc(-c%13ccccc%13)cc%12c%11c%10)c%10ccccc9%10)c8c7c6)c6ccccc56)cc4c3c2)cc1 ; c1cc(-c2c3ccccc3c(-n3c4ccccc4c4ccccc43)c3ccccc23)c2c(c1)[se]c1ccc(-c3c4ccccc4c(-n4c5ccccc5c5ccccc54)c4ccccc34)cc12 ; CC(C)(C)c1ccc2c(c1)-c1cc(C(C)(C)C)ccc1C2c1c2ccccc2c(-c2cccc3[se]c4ccc(-c5c6ccccc6c(-n6c7ccc(C(C)(C)C)cc7c7cc(C(C)(C)C)ccc76)c6ccccc56)cc4c23)c2ccccc12]

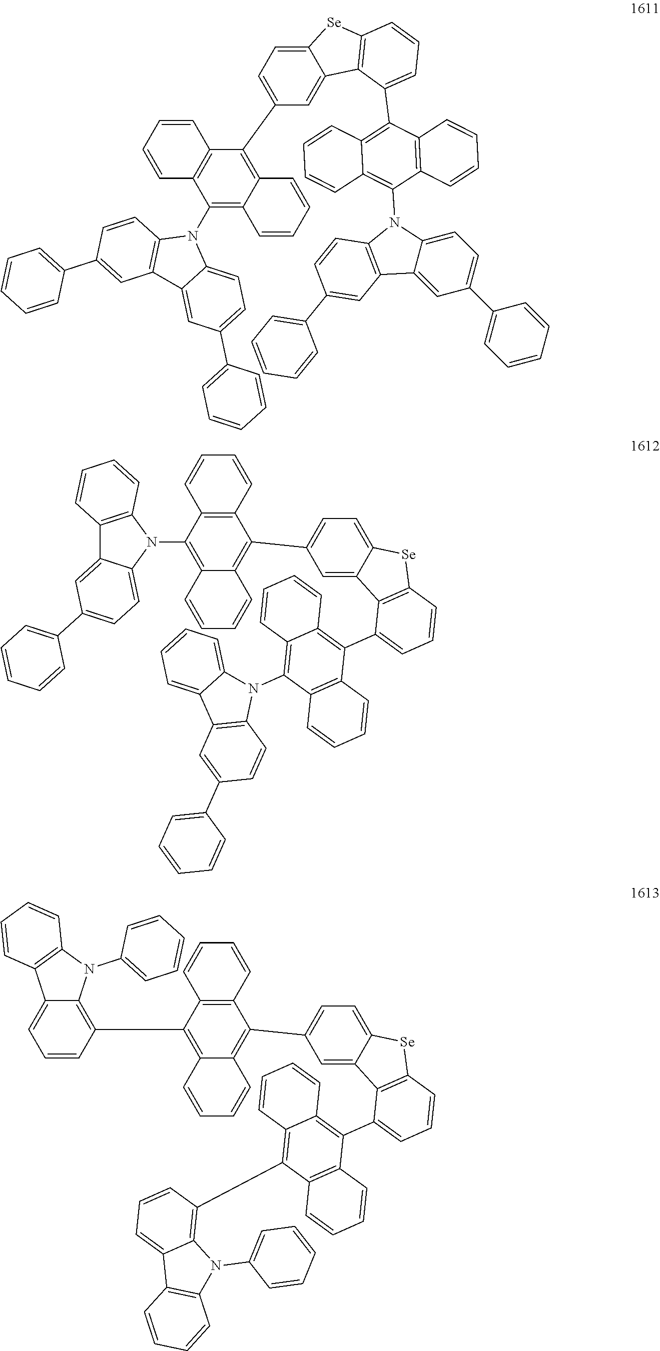

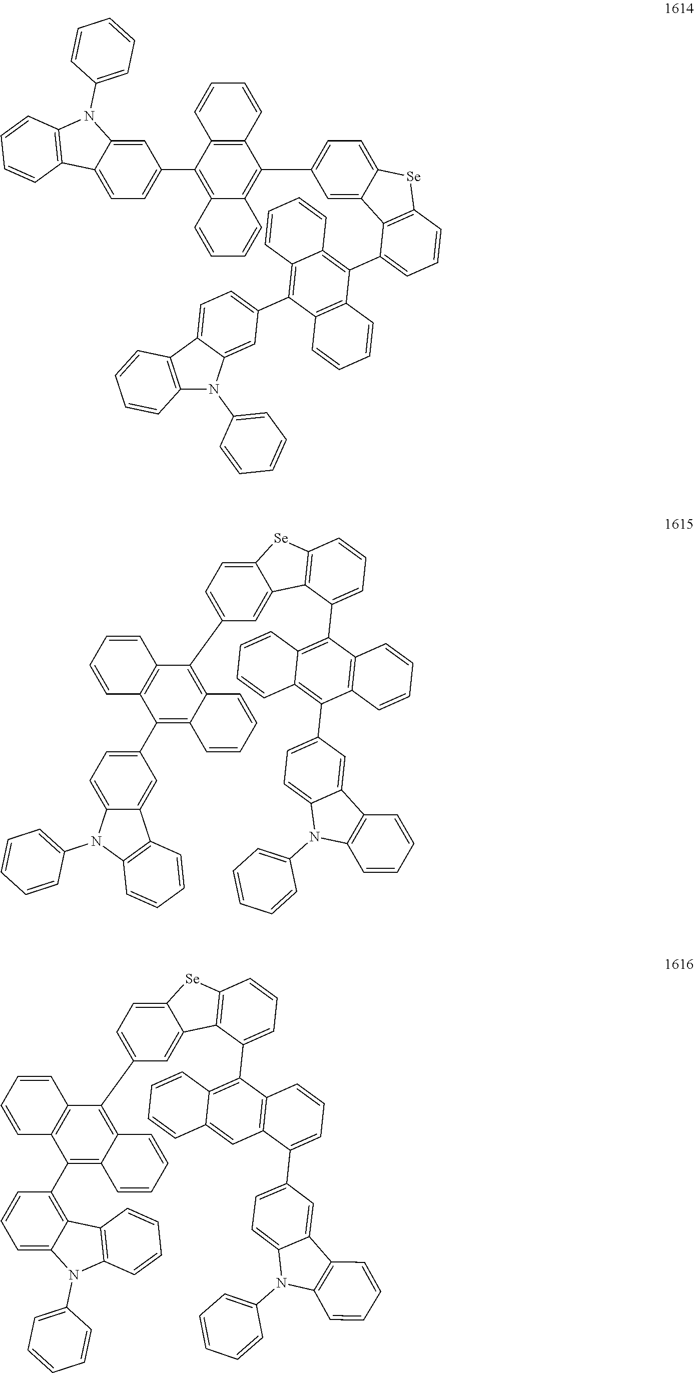 [SMILES: c1ccc(-n2c3ccccc3c3ccc(-c4c5ccccc5c(-c5ccc6[se]c7cccc(-c8c9ccccc9c(-c9ccc%10c%11ccccc%11n(-c%11ccccc%11)c%10c9)c9ccccc89)c7c6c5)c5ccccc45)cc32)cc1 ; c1ccc(-n2c3ccccc3c3cc(-c4c5ccccc5c(-c5ccc6[se]c7cccc(-c8c9ccccc9c(-c9ccc%10c(c9)c9ccccc9n%10-c9ccccc9)c9ccccc89)c7c6c5)c5ccccc45)ccc32)cc1 ; c1ccc(-n2c3ccccc3c3cc(-c4cccc5c(-c6cccc7[se]c8ccc(-c9c%10ccccc%10c(-c%10cccc%11c%10c%10ccccc%10n%11-c%10ccccc%10)c%10ccccc9%10)cc8c67)c6ccccc6cc45)ccc32)cc1]

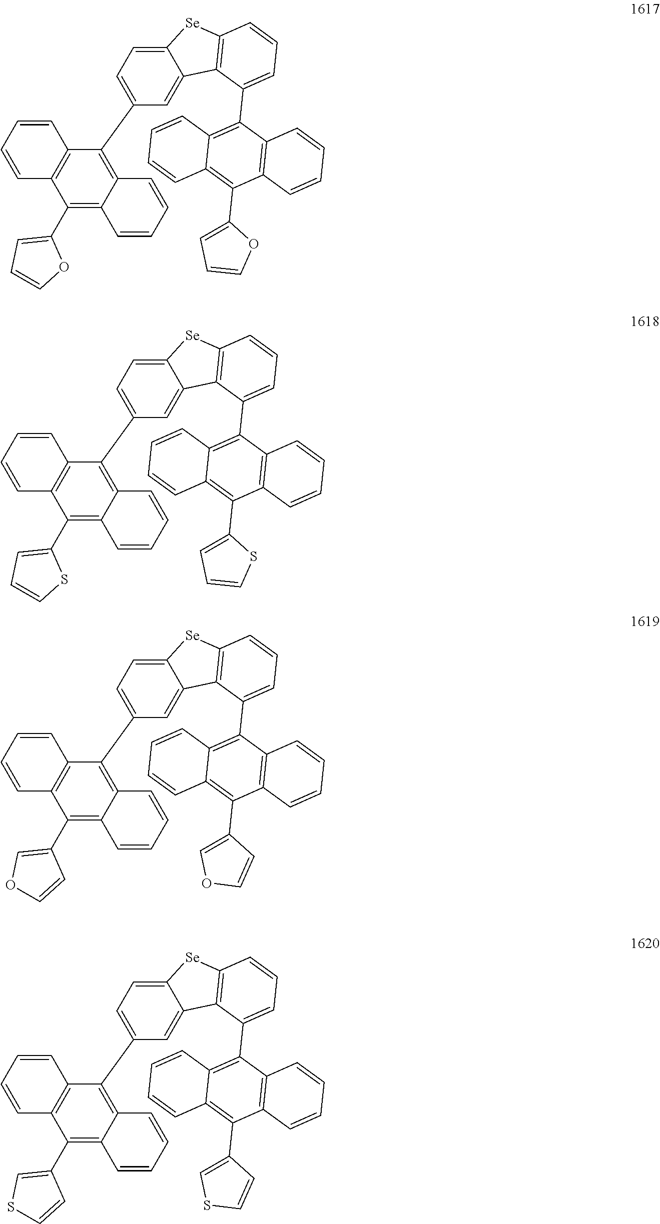 [SMILES: c1coc(-c2c3ccccc3c(-c3ccc4[se]c5cccc(-c6c7ccccc7c(-c7ccco7)c7ccccc67)c5c4c3)c3ccccc23)c1 ; c1csc(-c2c3ccccc3c(-c3ccc4[se]c5cccc(-c6c7ccccc7c(-c7cccs7)c7ccccc67)c5c4c3)c3ccccc23)c1 ; c1cc(-c2c3ccccc3c(-c3ccoc3)c3ccccc23)c2c(c1)[se]c1ccc(-c3c4ccccc4c(-c4ccoc4)c4ccccc34)cc12 ; c1cc(-c2c3ccccc3c(-c3ccsc3)c3ccccc23)c2c(c1)[se]c1ccc(-c3c4ccccc4c(-c4ccsc4)c4ccccc34)cc12]

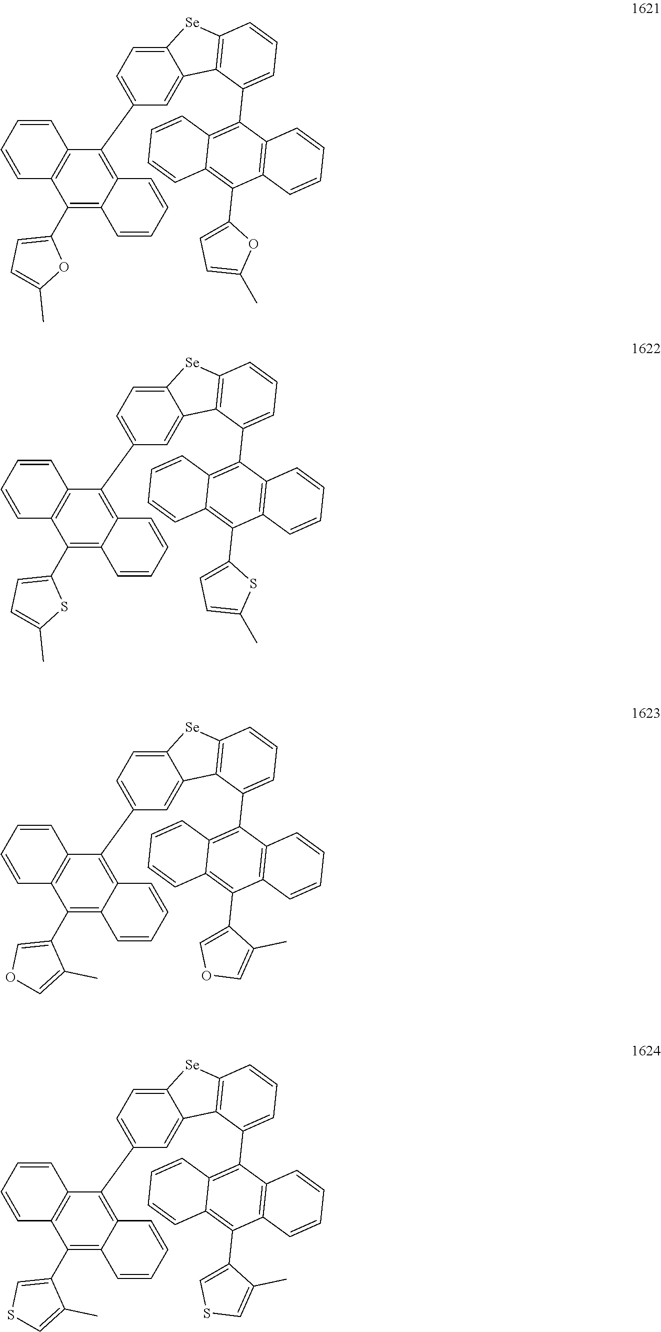 [SMILES: Cc1ccc(-c2c3ccccc3c(-c3ccc4[se]c5cccc(-c6c7ccccc7c(-c7ccc(C)o7)c7ccccc67)c5c4c3)c3ccccc23)o1 ; Cc1ccc(-c2c3ccccc3c(-c3ccc4[se]c5cccc(-c6c7ccccc7c(-c7ccc(C)s7)c7ccccc67)c5c4c3)c3ccccc23)s1 ; Cc1cocc1-c1c2ccccc2c(-c2ccc3[se]c4cccc(-c5c6ccccc6c(-c6cocc6C)c6ccccc56)c4c3c2)c2ccccc12 ; Cc1cscc1-c1c2ccccc2c(-c2ccc3[se]c4cccc(-c5c6ccccc6c(-c6cscc6C)c6ccccc56)c4c3c2)c2ccccc12]

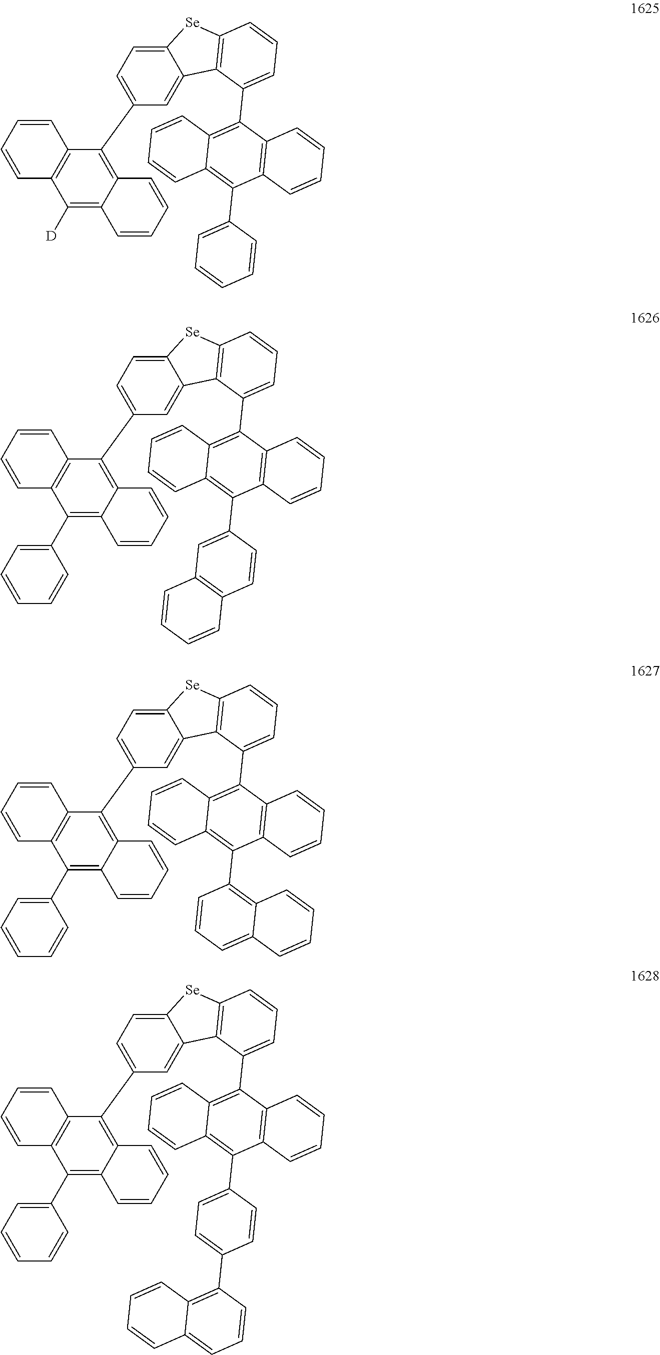

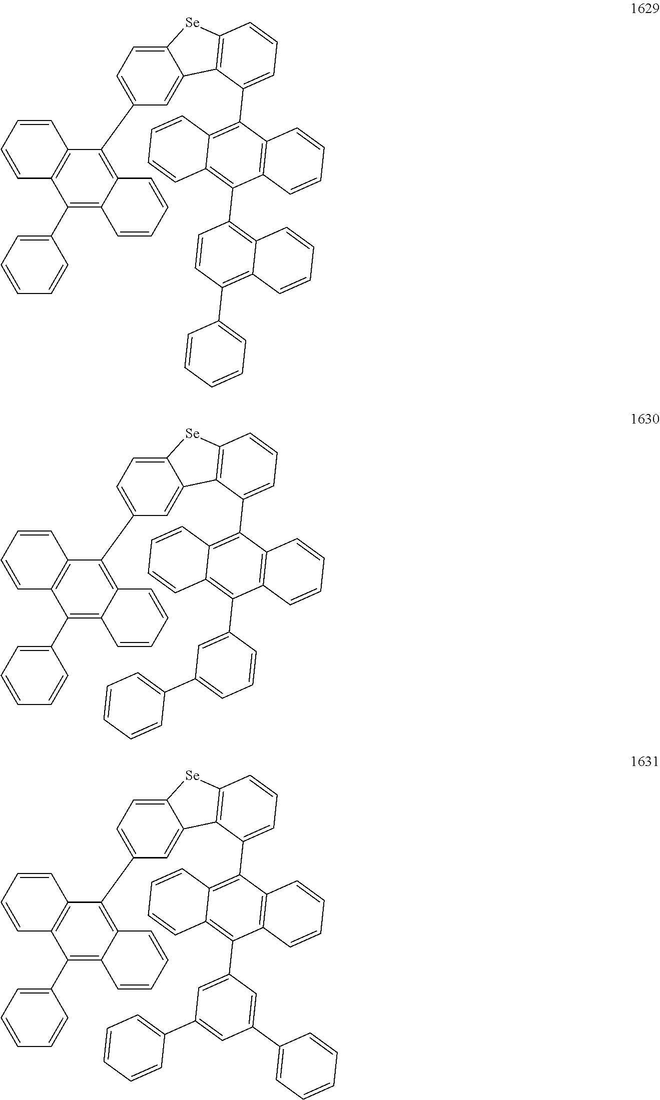 [SMILES: c1ccc(-c2ccc(-c3c4ccccc4c(-c4cccc5[se]c6ccc(-c7c8ccccc8c(-c8ccccc8)c8ccccc78)cc6c45)c4ccccc34)c3ccccc23)cc1 ; c1ccc(-c2cccc(-c3c4ccccc4c(-c4cccc5[se]c6ccc(-c7c8ccccc8c(-c8ccccc8)c8ccccc78)cc6c45)c4ccccc34)c2)cc1 ; c1ccc(-c2cc(-c3ccccc3)cc(-c3c4ccccc4c(-c4cccc5[se]c6ccc(-c7c8ccccc8c(-c8ccccc8)c8ccccc78)cc6c45)c4ccccc34)c2)cc1]

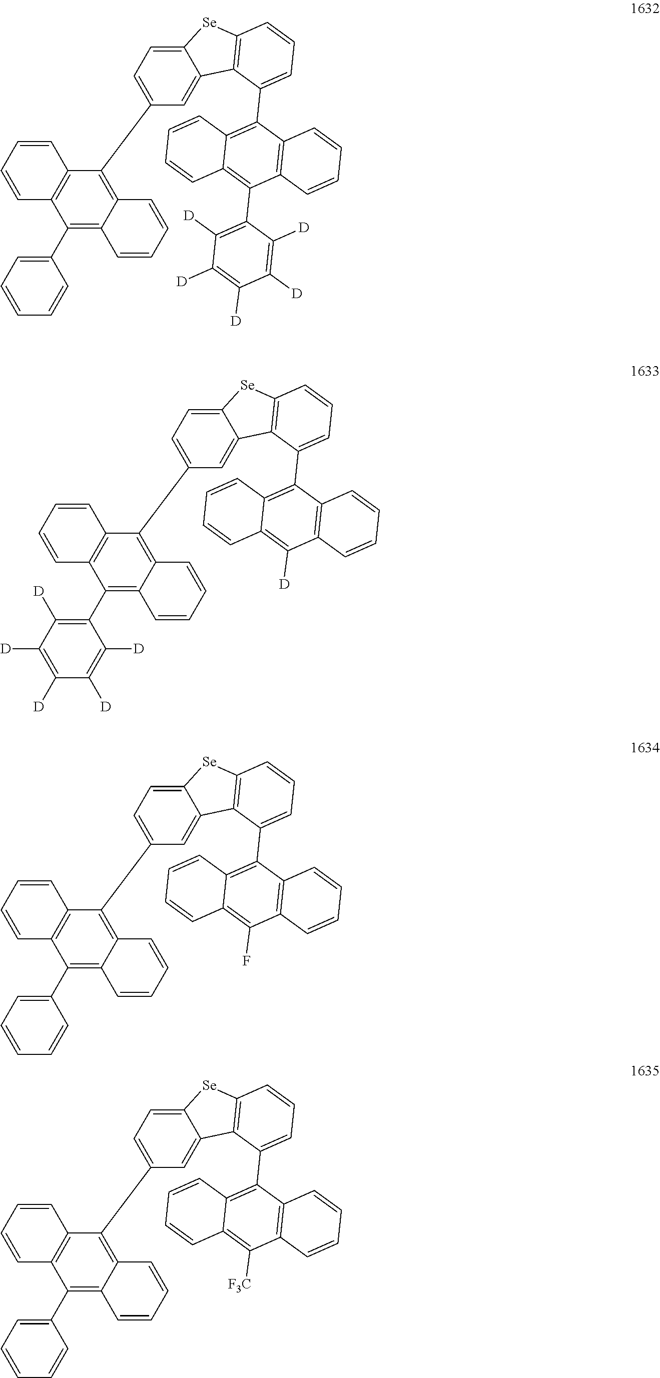 [SMILES: [2H]c1c([2H])c([2H])c(-c2c3ccccc3c(-c3cccc4[se]c5ccc(-c6c7ccccc7c(-c7ccccc7)c7ccccc67)cc5c34)c3ccccc23)c([2H])c1[2H] ; [2H]c1c([2H])c([2H])c(-c2c3ccccc3c(-c3ccc4[se]c5cccc(-c6c7ccccc7c([2H])c7ccccc67)c5c4c3)c3ccccc23)c([2H])c1[2H] ; Fc1c2ccccc2c(-c2cccc3[se]c4ccc(-c5c6ccccc6c(-c6ccccc6)c6ccccc56)cc4c23)c2ccccc12 ; FC(F)(F)c1c2ccccc2c(-c2cccc3[se]c4ccc(-c5c6ccccc6c(-c6ccccc6)c6ccccc56)cc4c23)c2ccccc12]

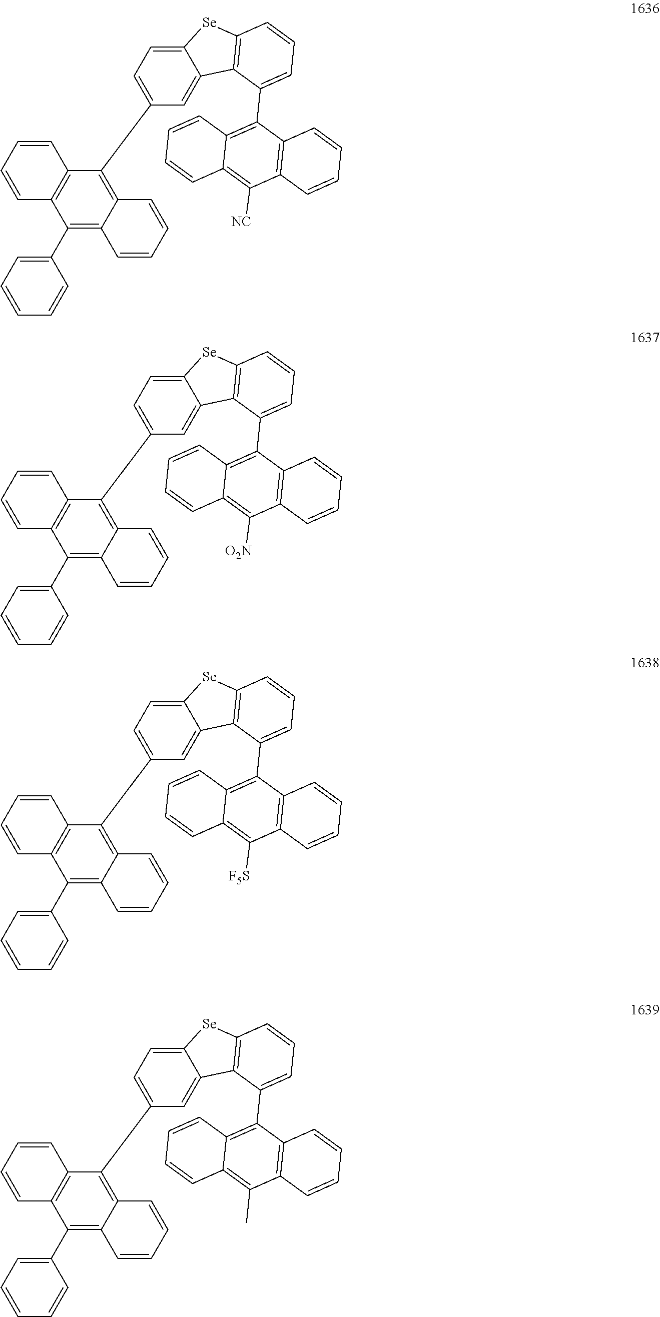

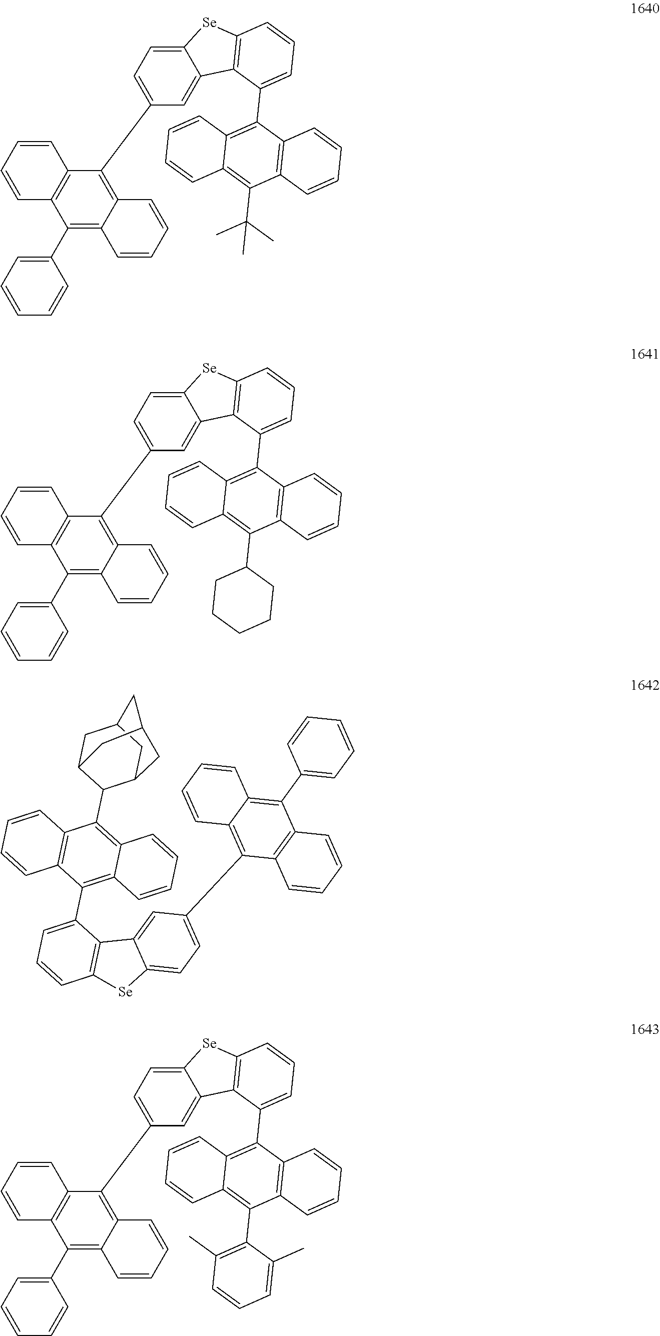 [SMILES: CC(C)(C)c1c2ccccc2c(-c2cccc3[se]c4ccc(-c5c6ccccc6c(-c6ccccc6)c6ccccc56)cc4c23)c2ccccc12 ; c1ccc(-c2c3ccccc3c(-c3ccc4[se]c5cccc(-c6c7ccccc7c(C7CCCCC7)c7ccccc67)c5c4c3)c3ccccc23)cc1 ; c1ccc(-c2c3ccccc3c(-c3ccc4[se]c5cccc(-c6c7ccccc7c(C7C8CC9CC(C8)CC7C9)c7ccccc67)c5c4c3)c3ccccc23)cc1 ; Cc1cccc(C)c1-c1c2ccccc2c(-c2cccc3[se]c4ccc(-c5c6ccccc6c(-c6ccccc6)c6ccccc56)cc4c23)c2ccccc12]

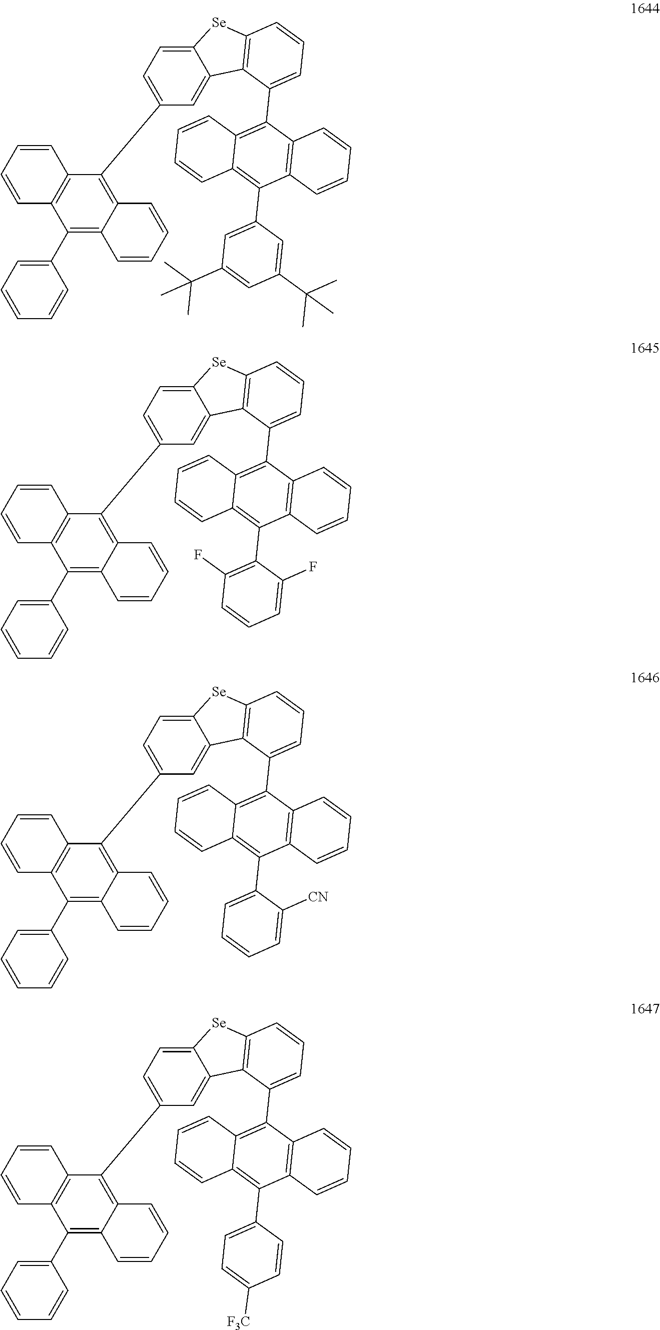

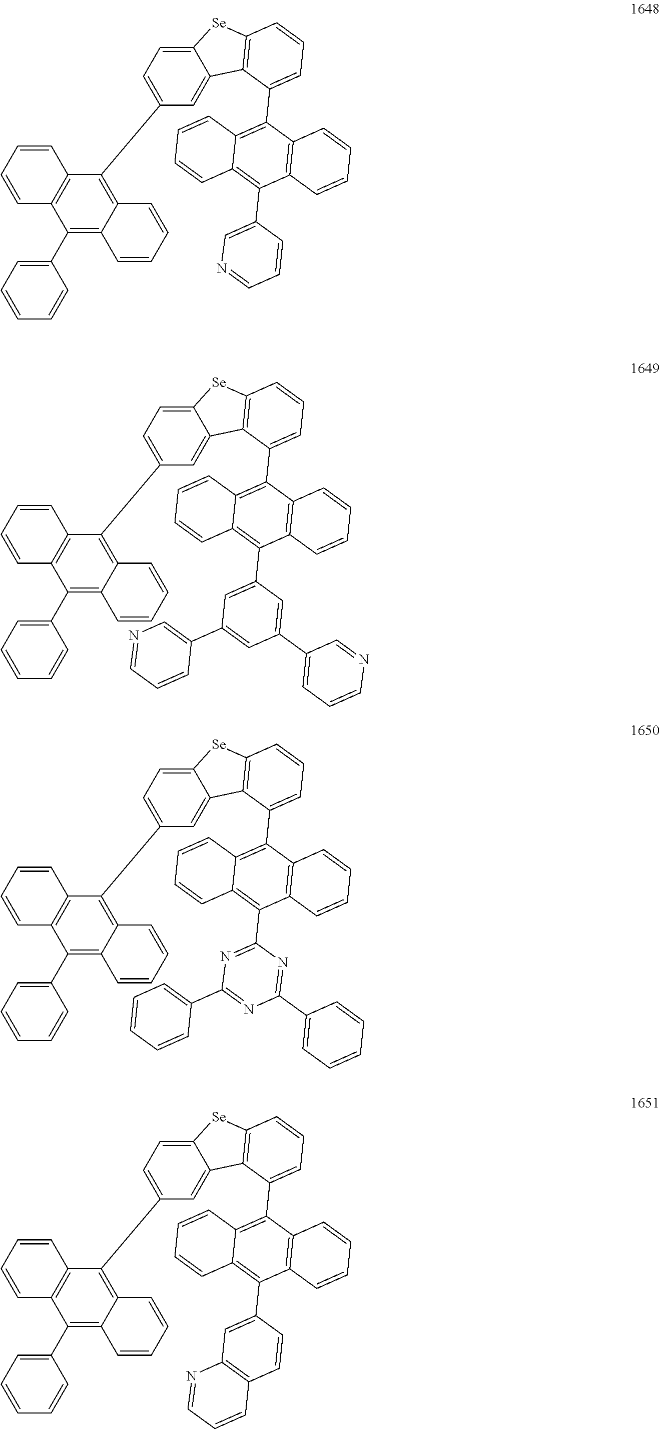 [SMILES: c1ccc(-c2c3ccccc3c(-c3ccc4[se]c5cccc(-c6c7ccccc7c(-c7cccnc7)c7ccccc67)c5c4c3)c3ccccc23)cc1 ; c1ccc(-c2c3ccccc3c(-c3ccc4[se]c5cccc(-c6c7ccccc7c(-c7cc(-c8cccnc8)cc(-c8cccnc8)c7)c7ccccc67)c5c4c3)c3ccccc23)cc1 ; c1ccc(-c2nc(-c3ccccc3)nc(-c3c4ccccc4c(-c4cccc5[se]c6ccc(-c7c8ccccc8c(-c8ccccc8)c8ccccc78)cc6c45)c4ccccc34)n2)cc1 ; c1ccc(-c2c3ccccc3c(-c3ccc4[se]c5cccc(-c6c7ccccc7c(-c7ccc8cccnc8c7)c7ccccc67)c5c4c3)c3ccccc23)cc1]

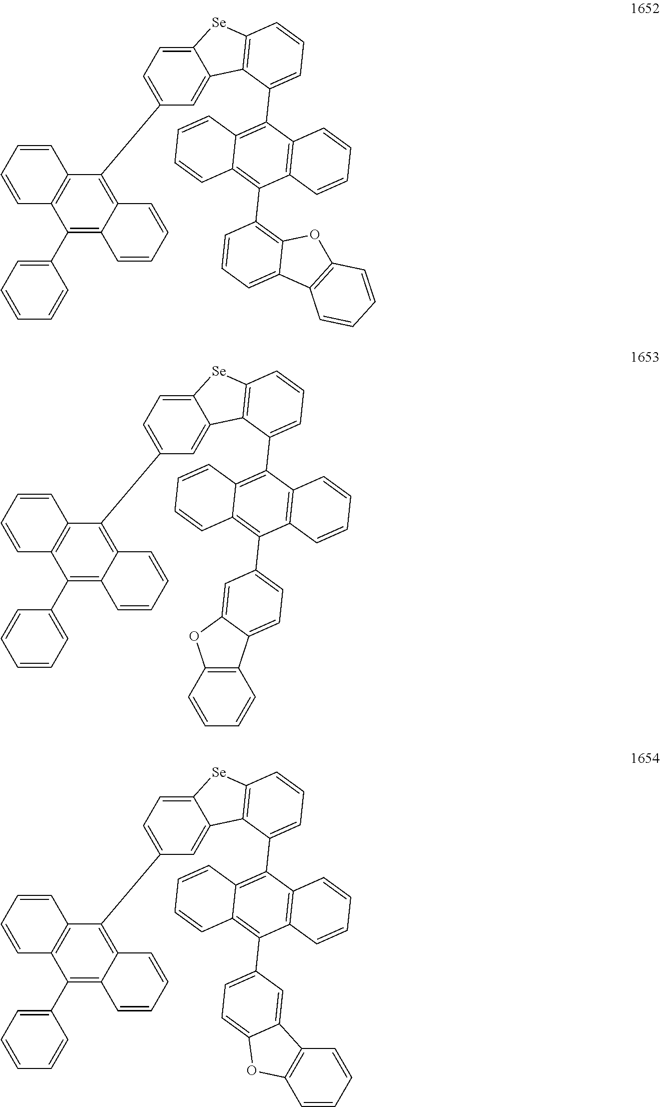 [SMILES: c1ccc(-c2c3ccccc3c(-c3ccc4[se]c5cccc(-c6c7ccccc7c(-c7cccc8c7oc7ccccc78)c7ccccc67)c5c4c3)c3ccccc23)cc1 ; c1ccc(-c2c3ccccc3c(-c3ccc4[se]c5cccc(-c6c7ccccc7c(-c7ccc8c(c7)oc7ccccc78)c7ccccc67)c5c4c3)c3ccccc23)cc1 ; c1ccc(-c2c3ccccc3c(-c3ccc4[se]c5cccc(-c6c7ccccc7c(-c7ccc8oc9ccccc9c8c7)c7ccccc67)c5c4c3)c3ccccc23)cc1]

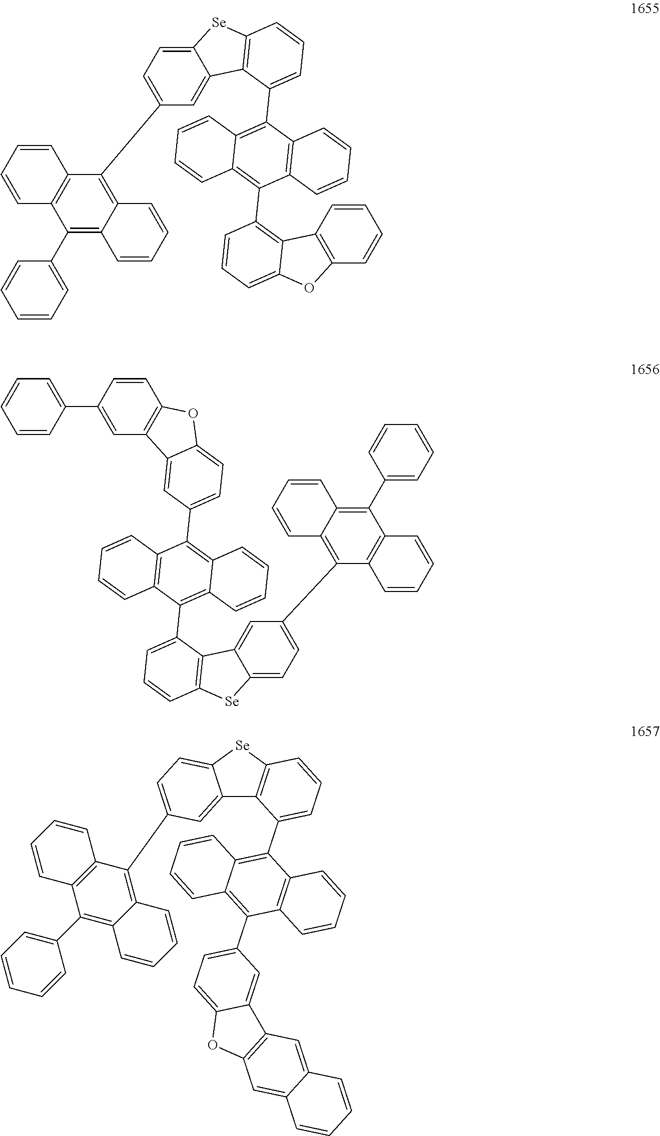

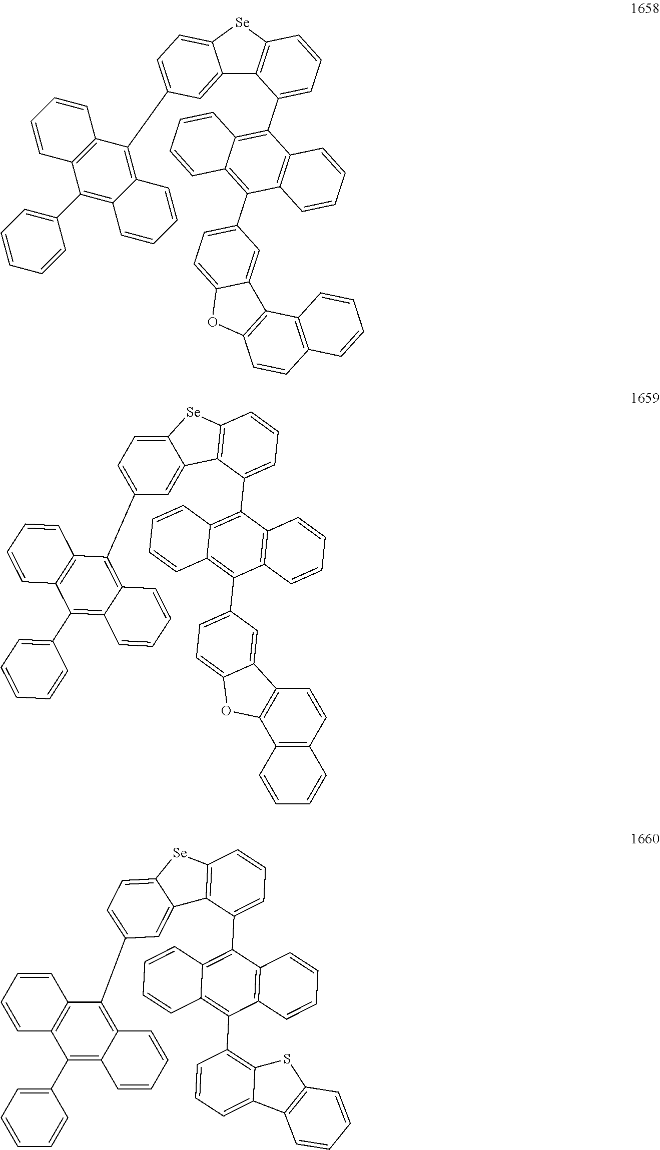

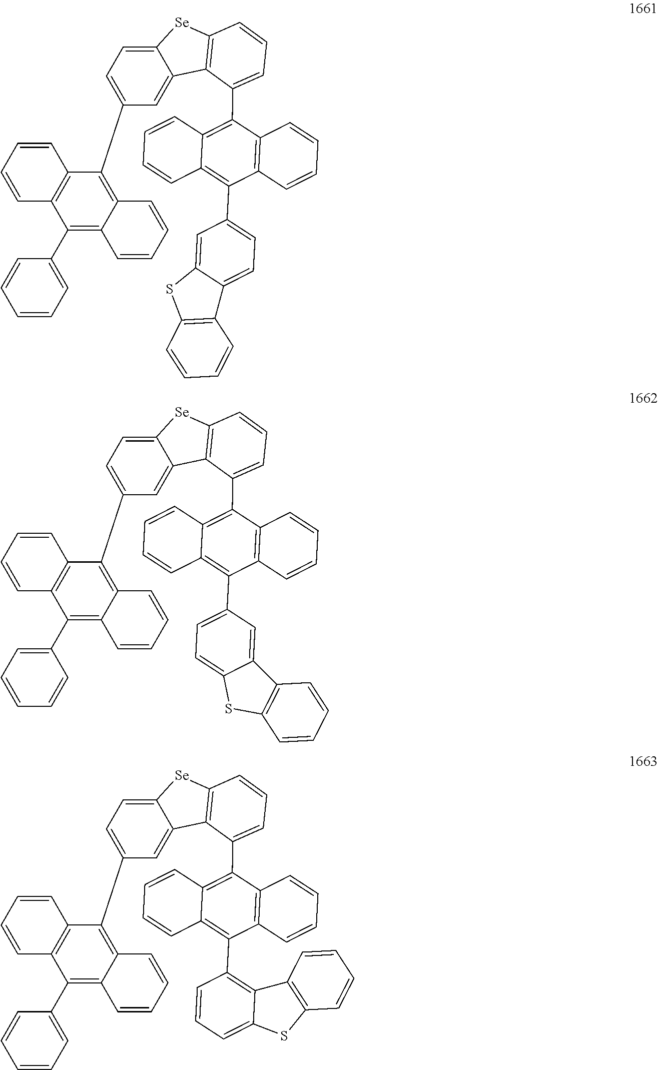

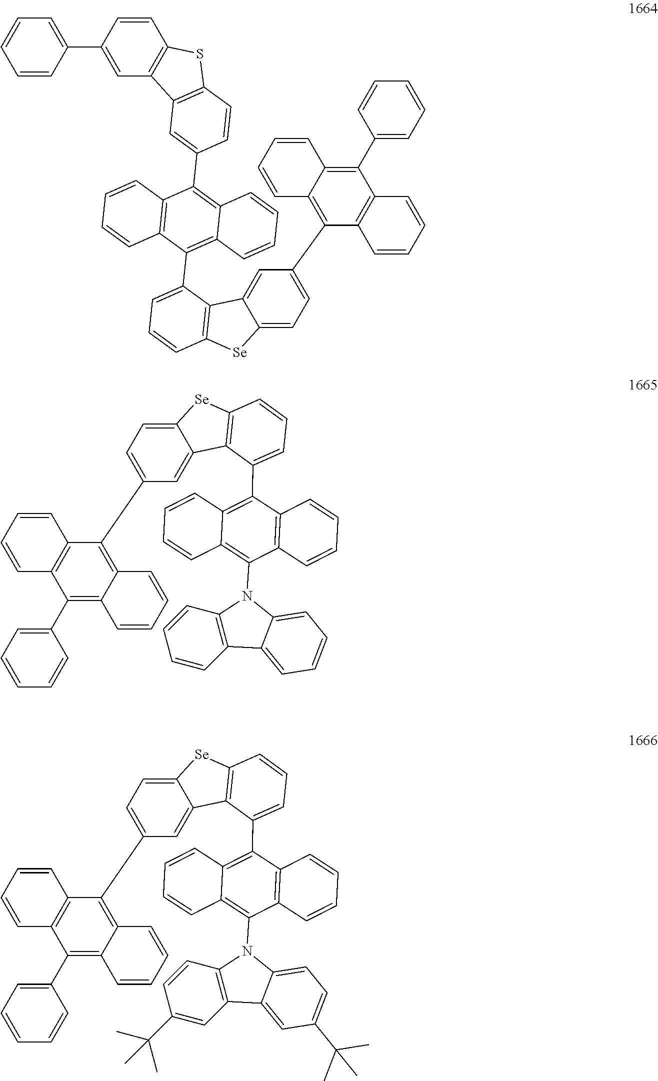 [SMILES: c1ccc(-c2ccc3sc4ccc(-c5c6ccccc6c(-c6cccc7[se]c8ccc(-c9c%10ccccc%10c(-c%10ccccc%10)c%10ccccc9%10)cc8c67)c6ccccc56)cc4c3c2)cc1 ; c1ccc(-c2c3ccccc3c(-c3ccc4[se]c5cccc(-c6c7ccccc7c(-n7c8ccccc8c8ccccc87)c7ccccc67)c5c4c3)c3ccccc23)cc1 ; CC(C)(C)c1ccc2c(c1)c1cc(C(C)(C)C)ccc1n2-c1c2ccccc2c(-c2cccc3[se]c4ccc(-c5c6ccccc6c(-c6ccccc6)c6ccccc56)cc4c23)c2ccccc12]

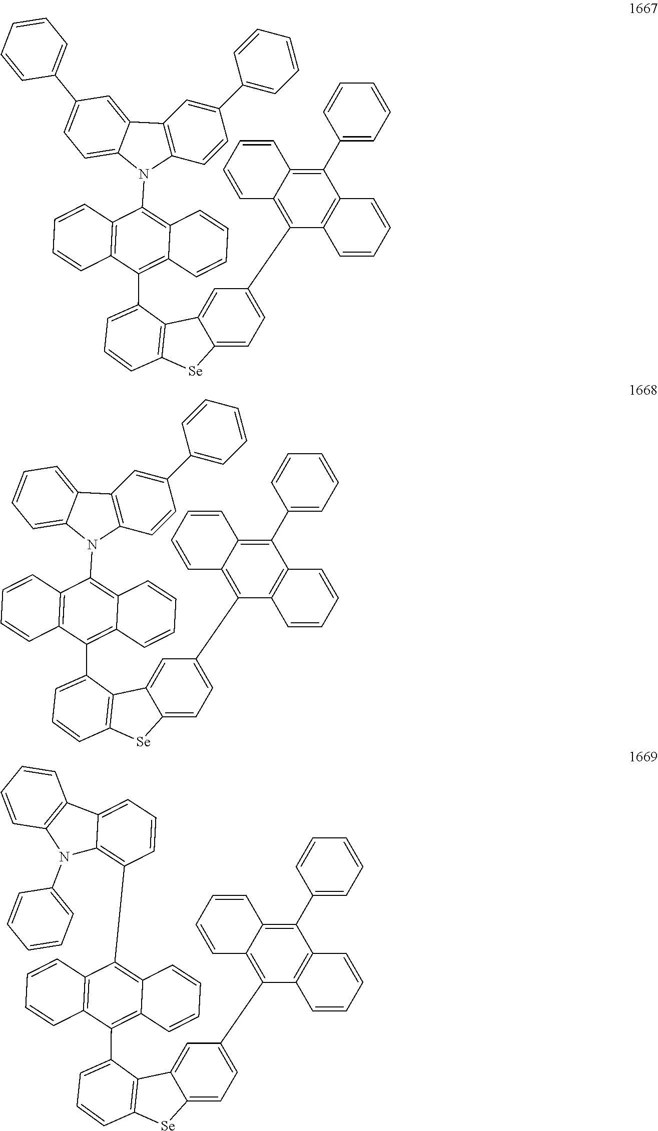

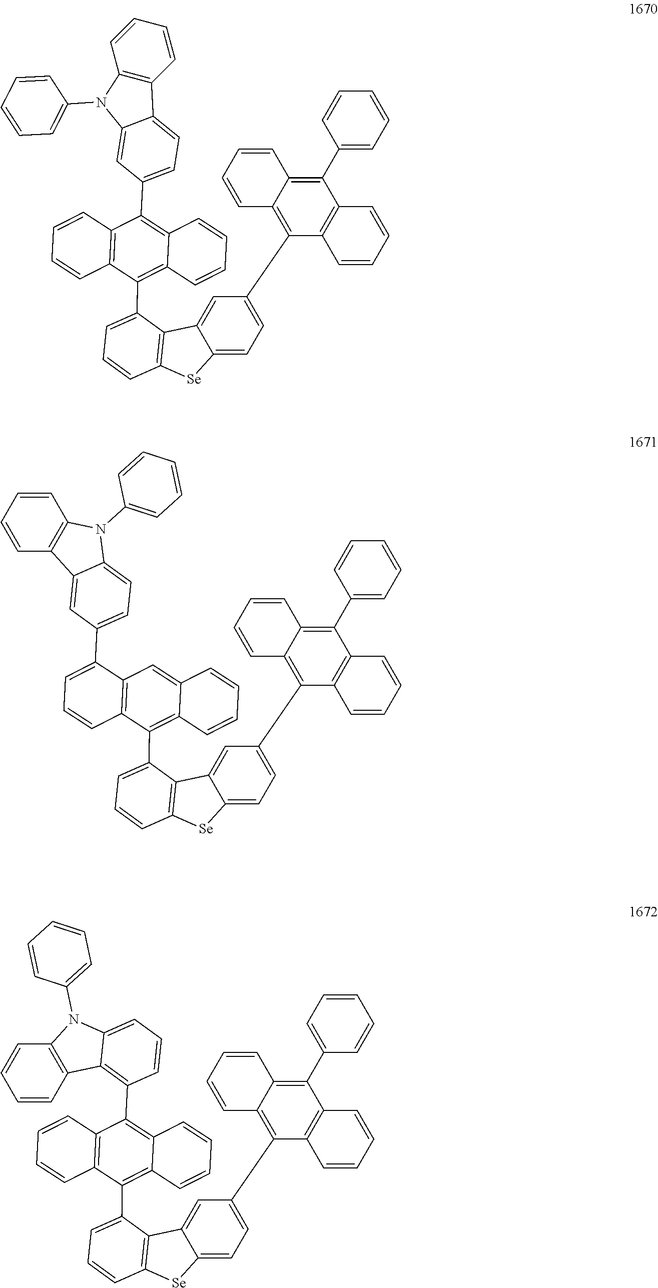 [SMILES: c1ccc(-c2c3ccccc3c(-c3ccc4[se]c5cccc(-c6c7ccccc7c(-c7ccc8c9ccccc9n(-c9ccccc9)c8c7)c7ccccc67)c5c4c3)c3ccccc23)cc1 ; c1ccc(-c2c3ccccc3c(-c3ccc4[se]c5cccc(-c6c7ccccc7cc7c(-c8ccc9c(c8)c8ccccc8n9-c8ccccc8)cccc67)c5c4c3)c3ccccc23)cc1 ; c1ccc(-c2c3ccccc3c(-c3ccc4[se]c5cccc(-c6c7ccccc7c(-c7cccc8c7c7ccccc7n8-c7ccccc7)c7ccccc67)c5c4c3)c3ccccc23)cc1]

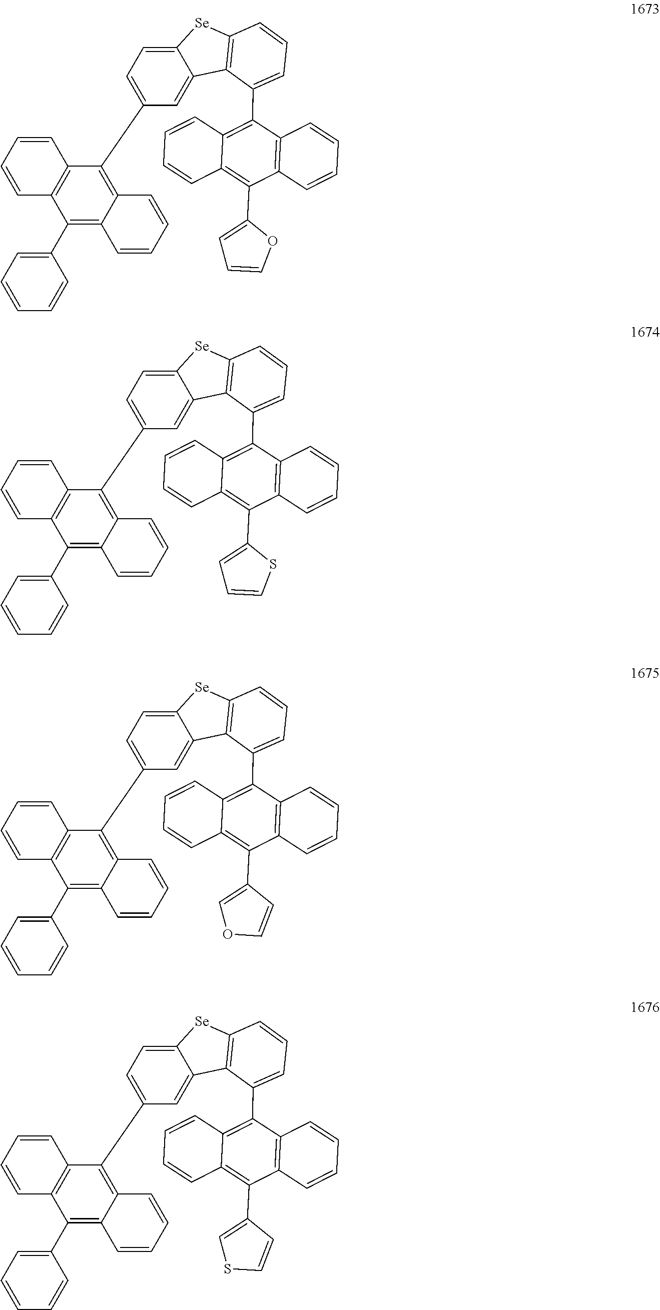 [SMILES: c1ccc(-c2c3ccccc3c(-c3ccc4[se]c5cccc(-c6c7ccccc7c(-c7ccco7)c7ccccc67)c5c4c3)c3ccccc23)cc1 ; c1ccc(-c2c3ccccc3c(-c3ccc4[se]c5cccc(-c6c7ccccc7c(-c7cccs7)c7ccccc67)c5c4c3)c3ccccc23)cc1 ; c1ccc(-c2c3ccccc3c(-c3ccc4[se]c5cccc(-c6c7ccccc7c(-c7ccoc7)c7ccccc67)c5c4c3)c3ccccc23)cc1 ; c1ccc(-c2c3ccccc3c(-c3ccc4[se]c5cccc(-c6c7ccccc7c(-c7ccsc7)c7ccccc67)c5c4c3)c3ccccc23)cc1]

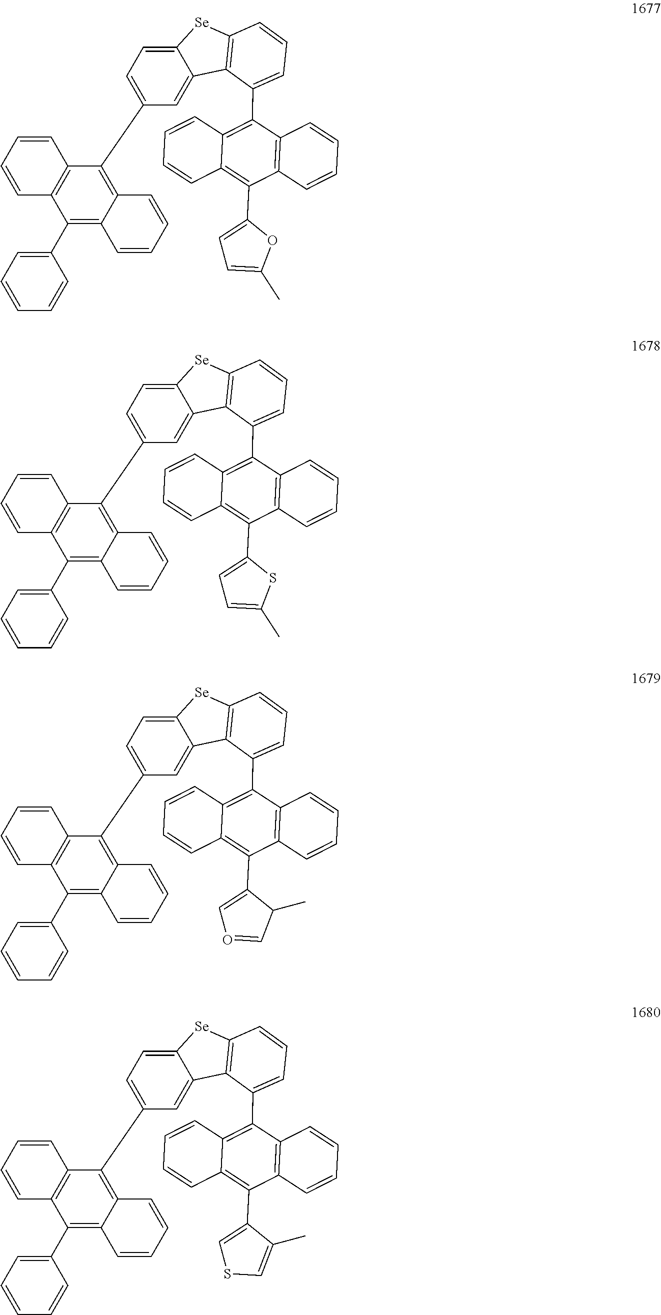

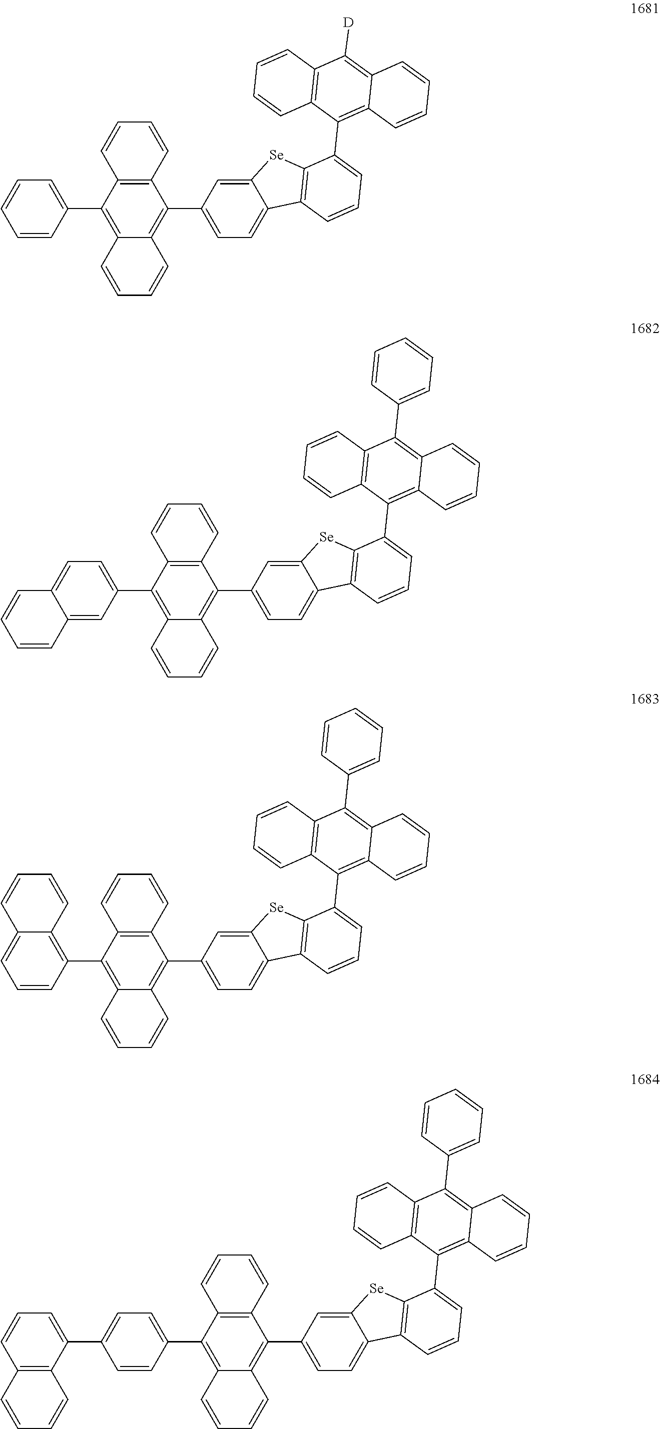 [SMILES: [2H]c1c2ccccc2c(-c2cccc3c2[se]c2cc(-c4c5ccccc5c(-c5ccccc5)c5ccccc45)ccc23)c2ccccc12 ; c1ccc(-c2c3ccccc3c(-c3cccc4c3[se]c3cc(-c5c6ccccc6c(-c6ccc7ccccc7c6)c6ccccc56)ccc34)c3ccccc23)cc1 ; c1ccc(-c2c3ccccc3c(-c3cccc4c3[se]c3cc(-c5c6ccccc6c(-c6cccc7ccccc67)c6ccccc56)ccc34)c3ccccc23)cc1 ; c1ccc(-c2c3ccccc3c(-c3cccc4c3[se]c3cc(-c5c6ccccc6c(-c6ccc(-c7cccc8ccccc78)cc6)c6ccccc56)ccc34)c3ccccc23)cc1]

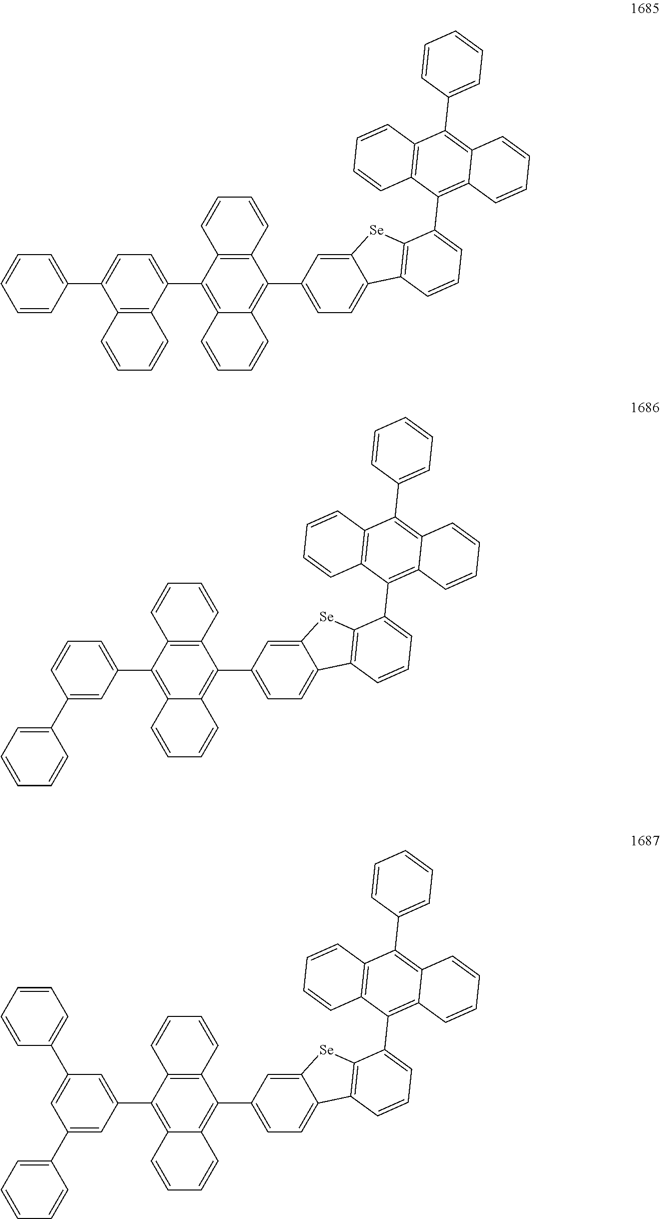 [SMILES: c1ccc(-c2ccc(-c3c4ccccc4c(-c4ccc5c(c4)[se]c4c(-c6c7ccccc7c(-c7ccccc7)c7ccccc67)cccc45)c4ccccc34)c3ccccc23)cc1 ; c1ccc(-c2cccc(-c3c4ccccc4c(-c4ccc5c(c4)[se]c4c(-c6c7ccccc7c(-c7ccccc7)c7ccccc67)cccc45)c4ccccc34)c2)cc1 ; c1ccc(-c2cc(-c3ccccc3)cc(-c3c4ccccc4c(-c4ccc5c(c4)[se]c4c(-c6c7ccccc7c(-c7ccccc7)c7ccccc67)cccc45)c4ccccc34)c2)cc1]

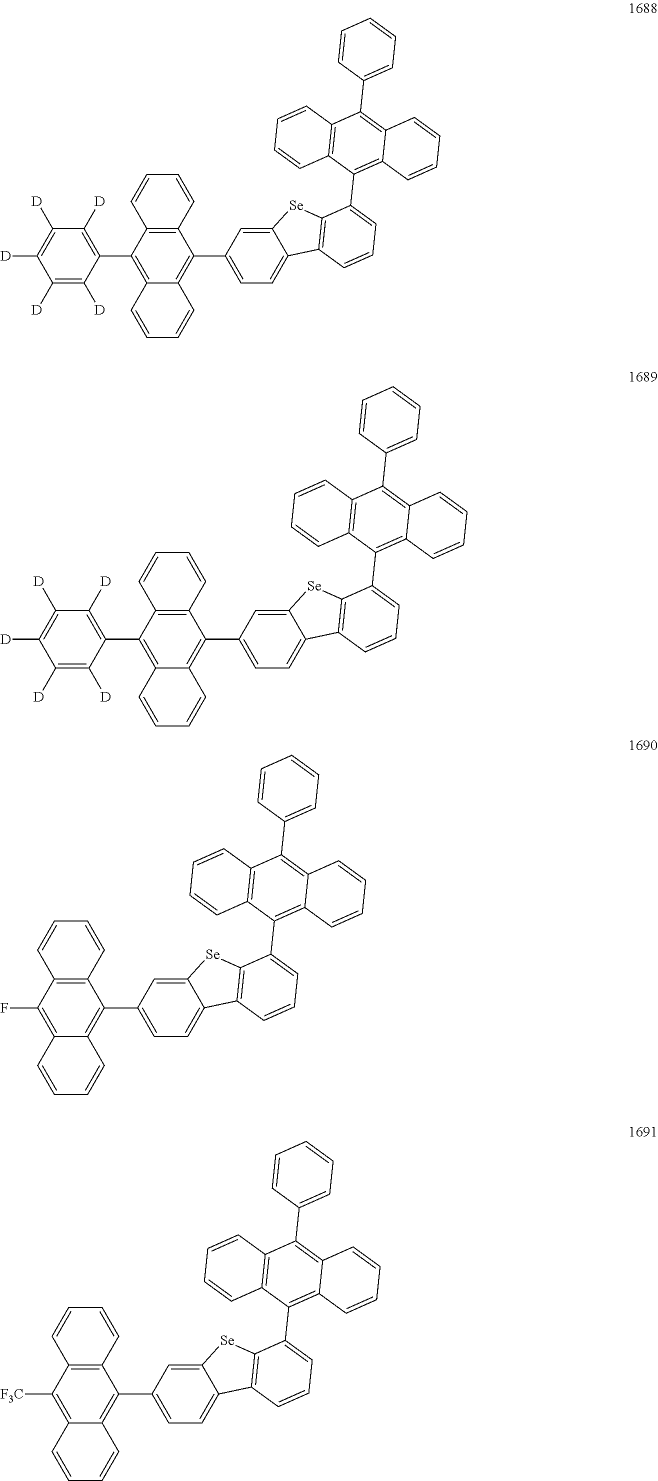 [SMILES: [2H]c1c([2H])c([2H])c(-c2c3ccccc3c(-c3ccc4c(c3)[se]c3c(-c5c6ccccc6c(-c6ccccc6)c6ccccc56)cccc34)c3ccccc23)c([2H])c1[2H] ; [2H]c1c([2H])c([2H])c(-c2c3ccccc3c(-c3ccc4c(c3)[se]c3c(-c5c6ccccc6c(-c6ccccc6)c6ccccc56)cccc34)c3ccccc23)c([2H])c1[2H] ; Fc1c2ccccc2c(-c2ccc3c(c2)[se]c2c(-c4c5ccccc5c(-c5ccccc5)c5ccccc45)cccc23)c2ccccc12 ; FC(F)(F)c1c2ccccc2c(-c2ccc3c(c2)[se]c2c(-c4c5ccccc5c(-c5ccccc5)c5ccccc45)cccc23)c2ccccc12]

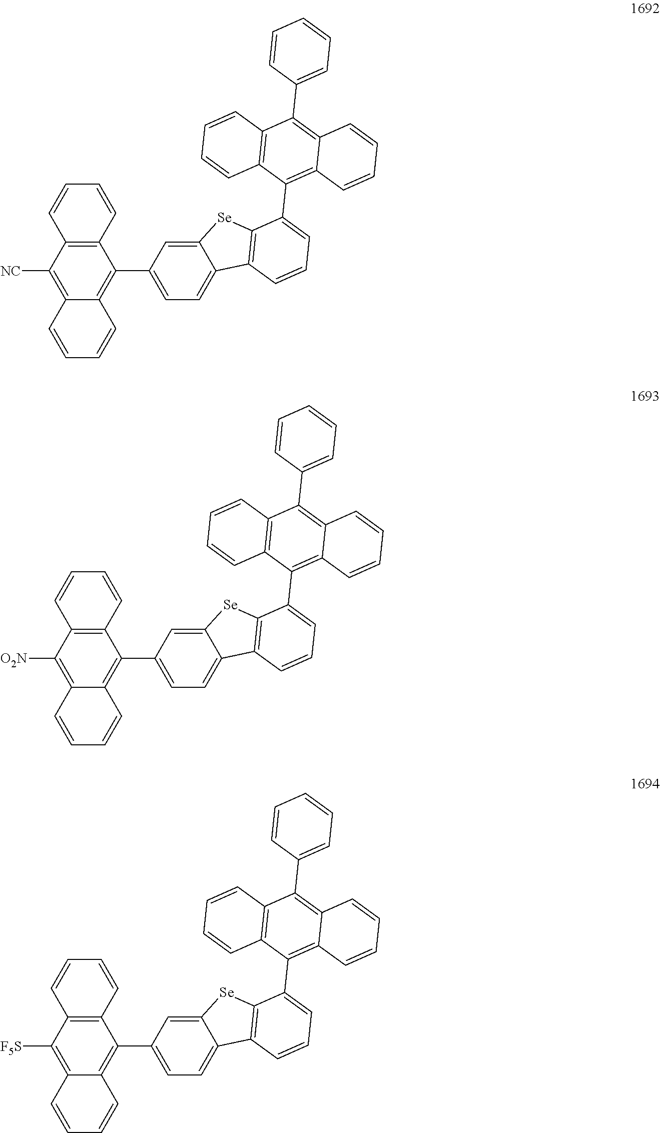

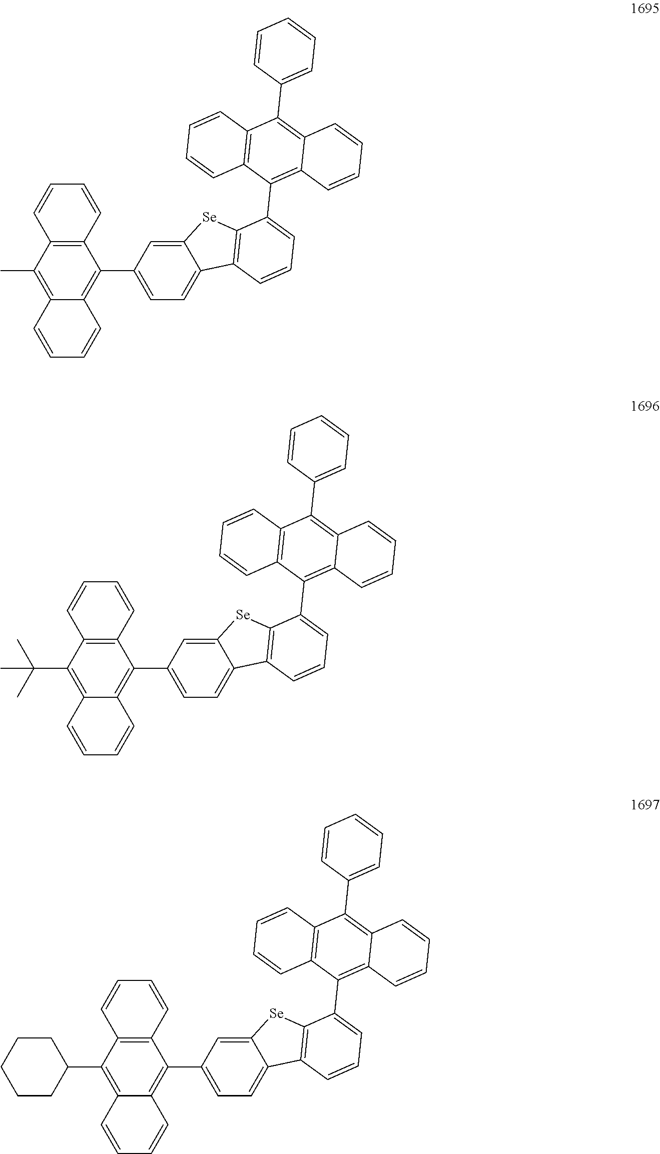 [SMILES: Cc1c2ccccc2c(-c2ccc3c(c2)[se]c2c(-c4c5ccccc5c(-c5ccccc5)c5ccccc45)cccc23)c2ccccc12 ; CC(C)(C)c1c2ccccc2c(-c2ccc3c(c2)[se]c2c(-c4c5ccccc5c(-c5ccccc5)c5ccccc45)cccc23)c2ccccc12 ; c1ccc(-c2c3ccccc3c(-c3cccc4c3[se]c3cc(-c5c6ccccc6c(C6CCCCC6)c6ccccc56)ccc34)c3ccccc23)cc1]

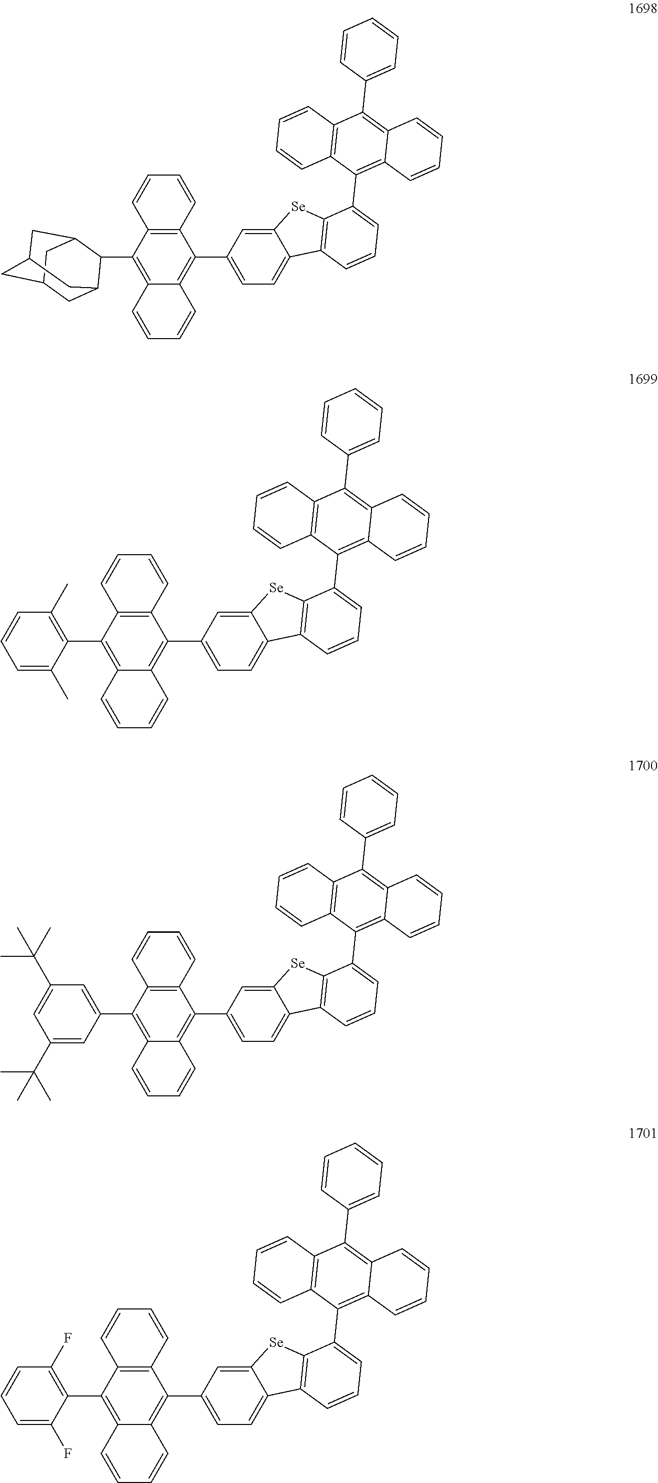 [SMILES: c1ccc(-c2c3ccccc3c(-c3cccc4c3[se]c3cc(-c5c6ccccc6c(C6C7CC8CC(C7)CC6C8)c6ccccc56)ccc34)c3ccccc23)cc1 ; Cc1cccc(C)c1-c1c2ccccc2c(-c2ccc3c(c2)[se]c2c(-c4c5ccccc5c(-c5ccccc5)c5ccccc45)cccc23)c2ccccc12 ; CC(C)(C)c1cc(-c2c3ccccc3c(-c3ccc4c(c3)[se]c3c(-c5c6ccccc6c(-c6ccccc6)c6ccccc56)cccc34)c3ccccc23)cc(C(C)(C)C)c1 ; Fc1cccc(F)c1-c1c2ccccc2c(-c2ccc3c(c2)[se]c2c(-c4c5ccccc5c(-c5ccccc5)c5ccccc45)cccc23)c2ccccc12]

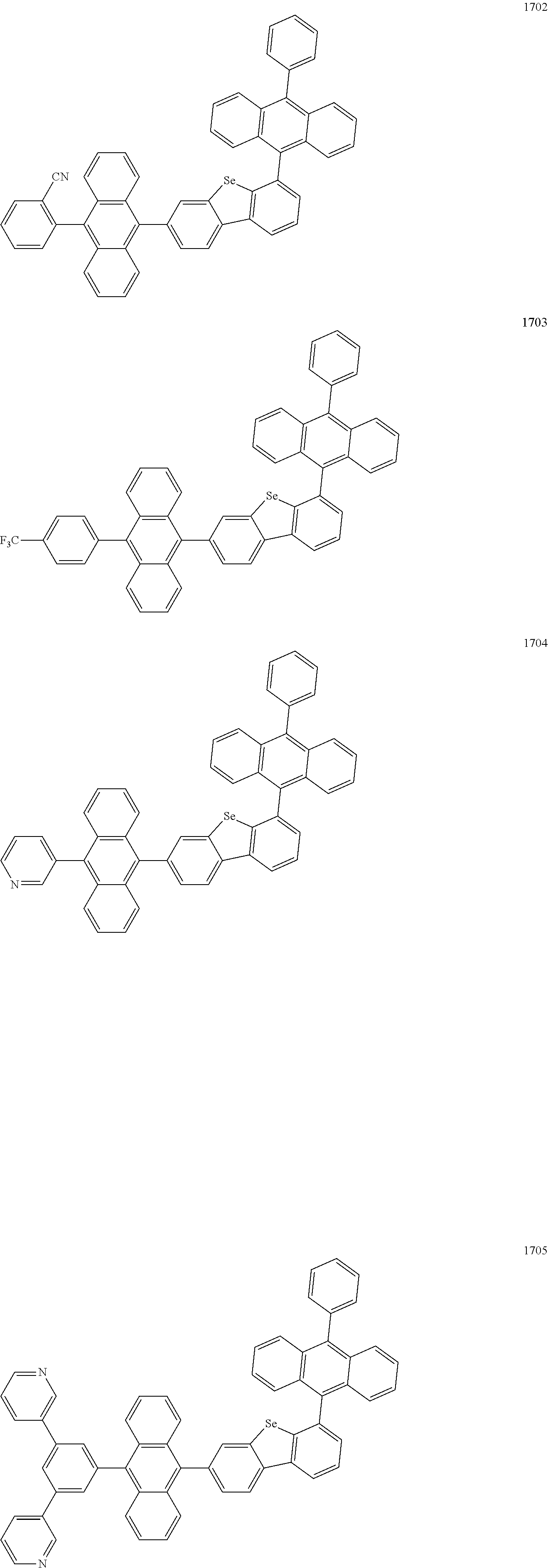 [SMILES: N#Cc1ccccc1-c1c2ccccc2c(-c2ccc3c(c2)[se]c2c(-c4c5ccccc5c(-c5ccccc5)c5ccccc45)cccc23)c2ccccc12 ; FC(F)(F)c1ccc(-c2c3ccccc3c(-c3ccc4c(c3)[se]c3c(-c5c6ccccc6c(-c6ccccc6)c6ccccc56)cccc34)c3ccccc23)cc1 ; c1ccc(-c2c3ccccc3c(-c3cccc4c3[se]c3cc(-c5c6ccccc6c(-c6cccnc6)c6ccccc56)ccc34)c3ccccc23)cc1 ; c1ccc(-c2c3ccccc3c(-c3cccc4c3[se]c3cc(-c5c6ccccc6c(-c6cc(-c7cccnc7)cc(-c7cccnc7)c6)c6ccccc56)ccc34)c3ccccc23)cc1]

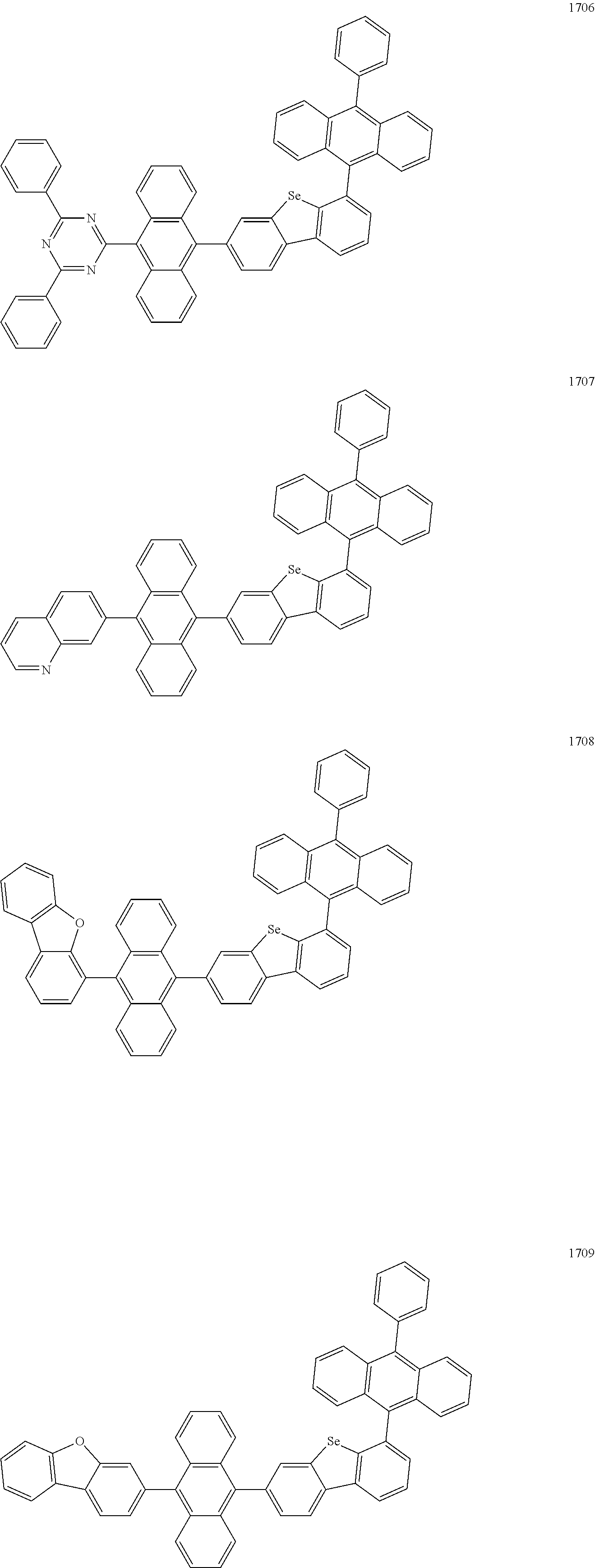 [SMILES: c1ccc(-c2nc(-c3ccccc3)nc(-c3c4ccccc4c(-c4ccc5c(c4)[se]c4c(-c6c7ccccc7c(-c7ccccc7)c7ccccc67)cccc45)c4ccccc34)n2)cc1 ; c1ccc(-c2c3ccccc3c(-c3cccc4c3[se]c3cc(-c5c6ccccc6c(-c6ccc7cccnc7c6)c6ccccc56)ccc34)c3ccccc23)cc1 ; c1ccc(-c2c3ccccc3c(-c3cccc4c3[se]c3cc(-c5c6ccccc6c(-c6cccc7c6oc6ccccc67)c6ccccc56)ccc34)c3ccccc23)cc1 ; c1ccc(-c2c3ccccc3c(-c3cccc4c3[se]c3cc(-c5c6ccccc6c(-c6ccc7c(c6)oc6ccccc67)c6ccccc56)ccc34)c3ccccc23)cc1]

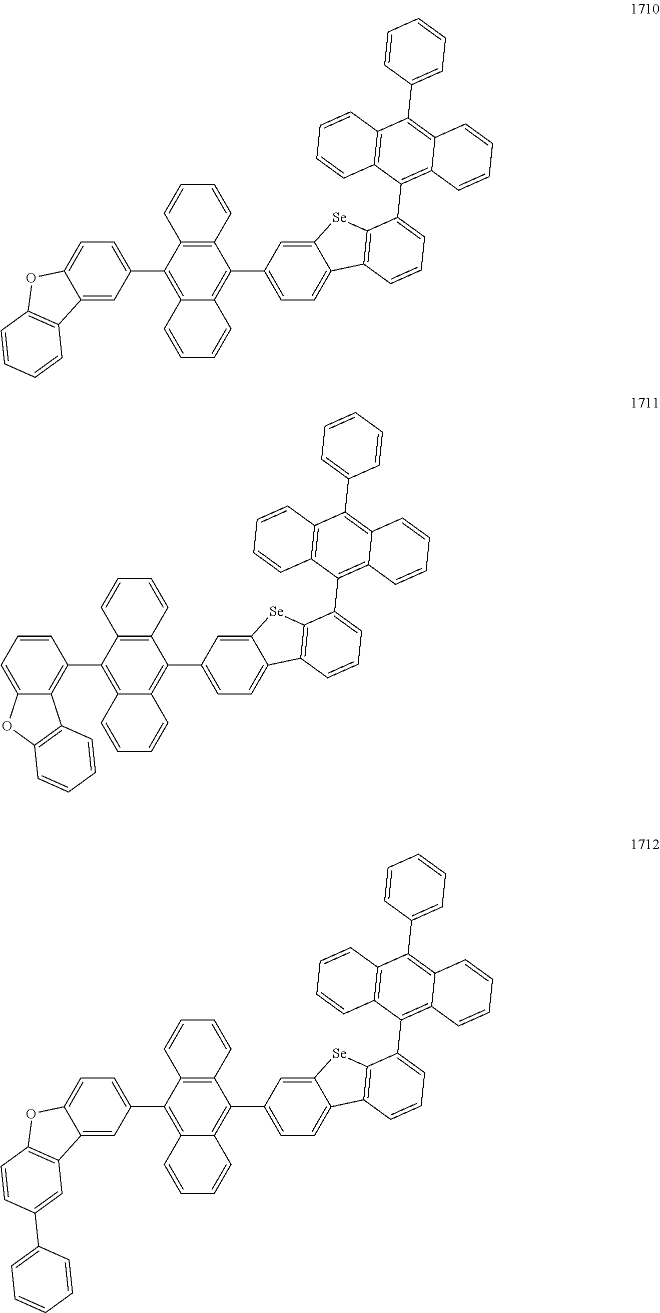 [SMILES: c1ccc(-c2c3ccccc3c(-c3cccc4c3[se]c3cc(-c5c6ccccc6c(-c6ccc7oc8ccccc8c7c6)c6ccccc56)ccc34)c3ccccc23)cc1 ; c1ccc(-c2c3ccccc3c(-c3cccc4c3[se]c3cc(-c5c6ccccc6c(-c6cccc7oc8ccccc8c67)c6ccccc56)ccc34)c3ccccc23)cc1 ; c1ccc(-c2ccc3oc4ccc(-c5c6ccccc6c(-c6ccc7c(c6)[se]c6c(-c8c9ccccc9c(-c9ccccc9)c9ccccc89)cccc67)c6ccccc56)cc4c3c2)cc1]

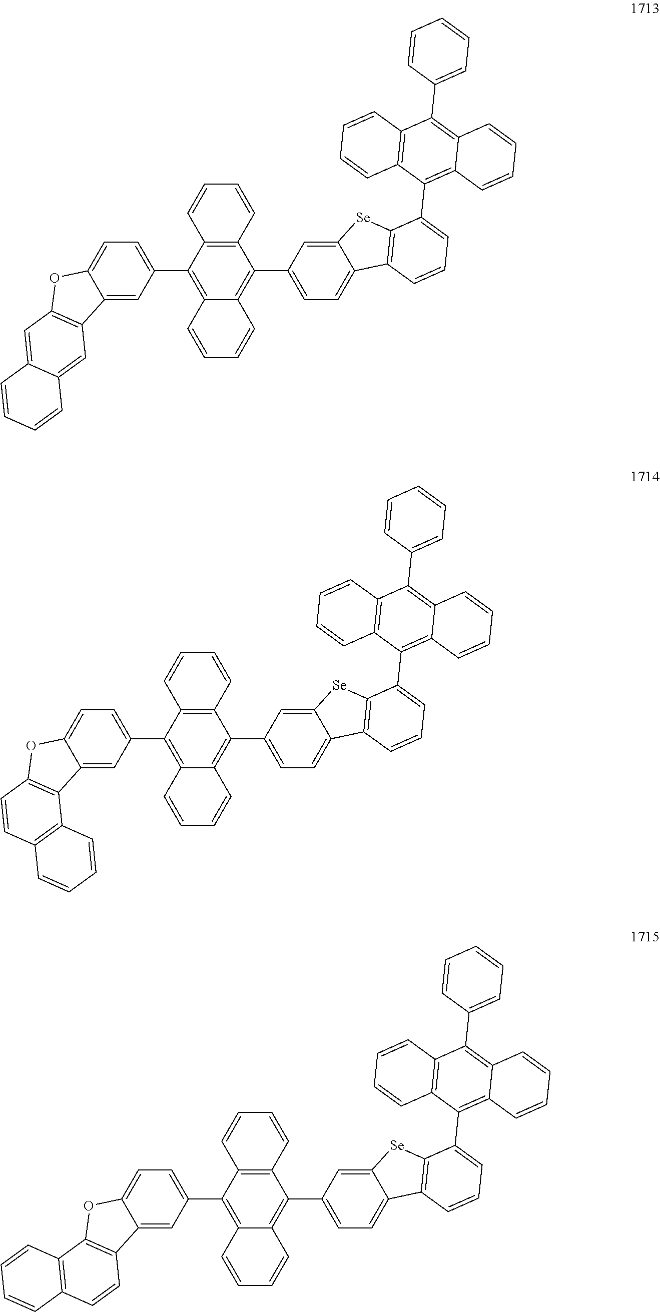 [SMILES: c1ccc(-c2c3ccccc3c(-c3cccc4c3[se]c3cc(-c5c6ccccc6c(-c6ccc7oc8cc9ccccc9cc8c7c6)c6ccccc56)ccc34)c3ccccc23)cc1 ; c1ccc(-c2c3ccccc3c(-c3cccc4c3[se]c3cc(-c5c6ccccc6c(-c6ccc7oc8ccc9ccccc9c8c7c6)c6ccccc56)ccc34)c3ccccc23)cc1 ; c1ccc(-c2c3ccccc3c(-c3cccc4c3[se]c3cc(-c5c6ccccc6c(-c6ccc7oc8c9ccccc9ccc8c7c6)c6ccccc56)ccc34)c3ccccc23)cc1]

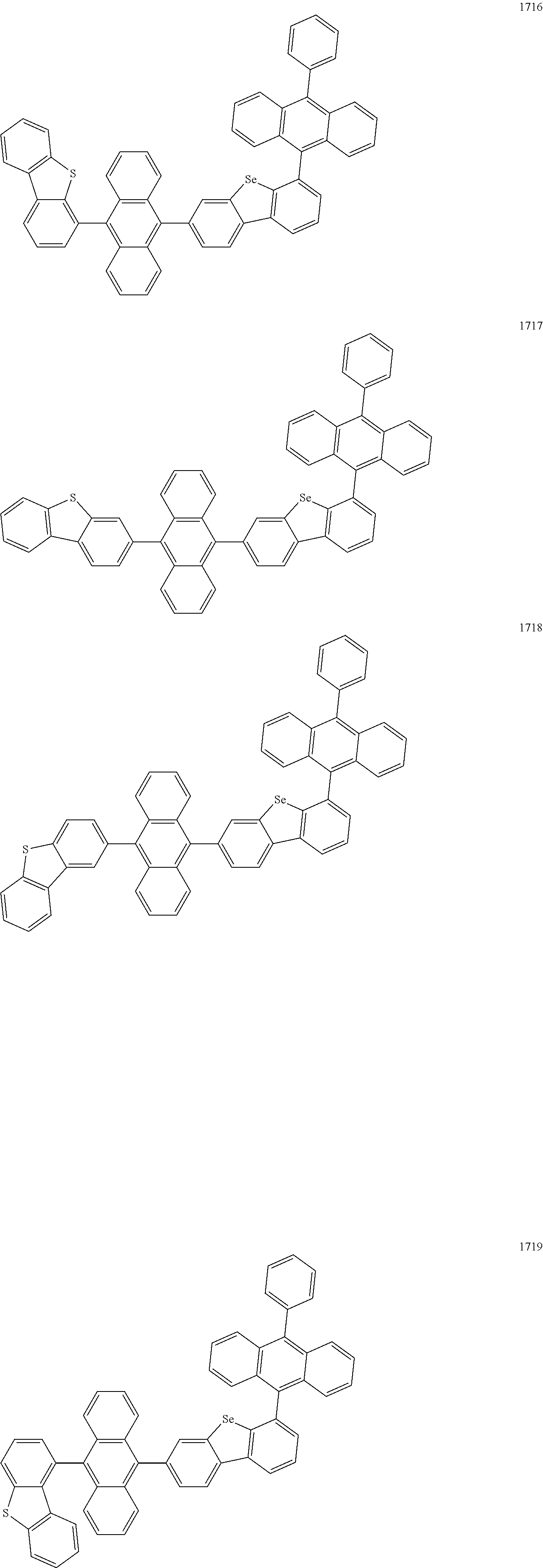 [SMILES: c1ccc(-c2c3ccccc3c(-c3cccc4c3[se]c3cc(-c5c6ccccc6c(-c6cccc7c6sc6ccccc67)c6ccccc56)ccc34)c3ccccc23)cc1 ; c1ccc(-c2c3ccccc3c(-c3cccc4c3[se]c3cc(-c5c6ccccc6c(-c6ccc7c(c6)sc6ccccc67)c6ccccc56)ccc34)c3ccccc23)cc1 ; c1ccc(-c2c3ccccc3c(-c3cccc4c3[se]c3cc(-c5c6ccccc6c(-c6ccc7sc8ccccc8c7c6)c6ccccc56)ccc34)c3ccccc23)cc1 ; c1ccc(-c2c3ccccc3c(-c3cccc4c3[se]c3cc(-c5c6ccccc6c(-c6cccc7sc8ccccc8c67)c6ccccc56)ccc34)c3ccccc23)cc1]

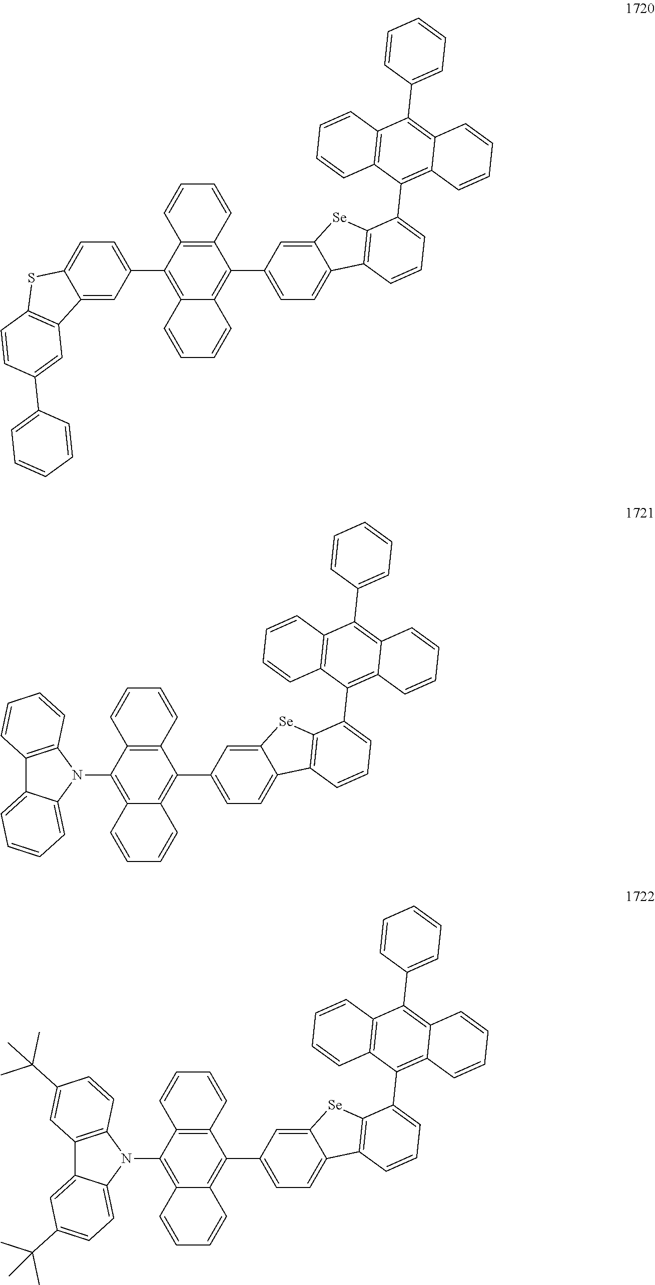 [SMILES: c1ccc(-c2ccc3sc4ccc(-c5c6ccccc6c(-c6ccc7c(c6)[se]c6c(-c8c9ccccc9c(-c9ccccc9)c9ccccc89)cccc67)c6ccccc56)cc4c3c2)cc1 ; c1ccc(-c2c3ccccc3c(-c3cccc4c3[se]c3cc(-c5c6ccccc6c(-n6c7ccccc7c7ccccc76)c6ccccc56)ccc34)c3ccccc23)cc1 ; CC(C)(C)c1ccc2c(c1)c1cc(C(C)(C)C)ccc1n2-c1c2ccccc2c(-c2ccc3c(c2)[se]c2c(-c4c5ccccc5c(-c5ccccc5)c5ccccc45)cccc23)c2ccccc12]

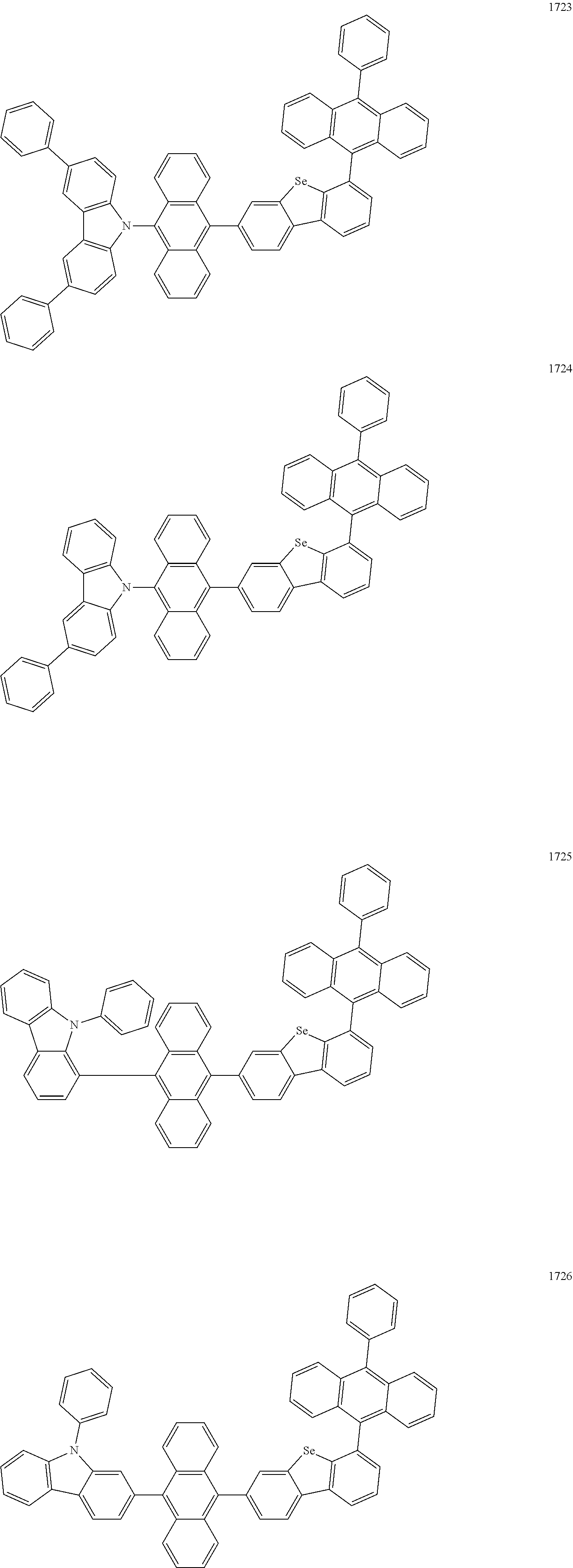 [SMILES: c1ccc(-c2ccc3c(c2)c2cc(-c4ccccc4)ccc2n3-c2c3ccccc3c(-c3ccc4c(c3)[se]c3c(-c5c6ccccc6c(-c6ccccc6)c6ccccc56)cccc34)c3ccccc23)cc1 ; c1ccc(-c2ccc3c(c2)c2ccccc2n3-c2c3ccccc3c(-c3ccc4c(c3)[se]c3c(-c5c6ccccc6c(-c6ccccc6)c6ccccc56)cccc34)c3ccccc23)cc1 ; c1ccc(-c2c3ccccc3c(-c3cccc4c3[se]c3cc(-c5c6ccccc6c(-c6cccc7c8ccccc8n(-c8ccccc8)c67)c6ccccc56)ccc34)c3ccccc23)cc1 ; c1ccc(-c2c3ccccc3c(-c3cccc4c3[se]c3cc(-c5c6ccccc6c(-c6ccc7c8ccccc8n(-c8ccccc8)c7c6)c6ccccc56)ccc34)c3ccccc23)cc1]

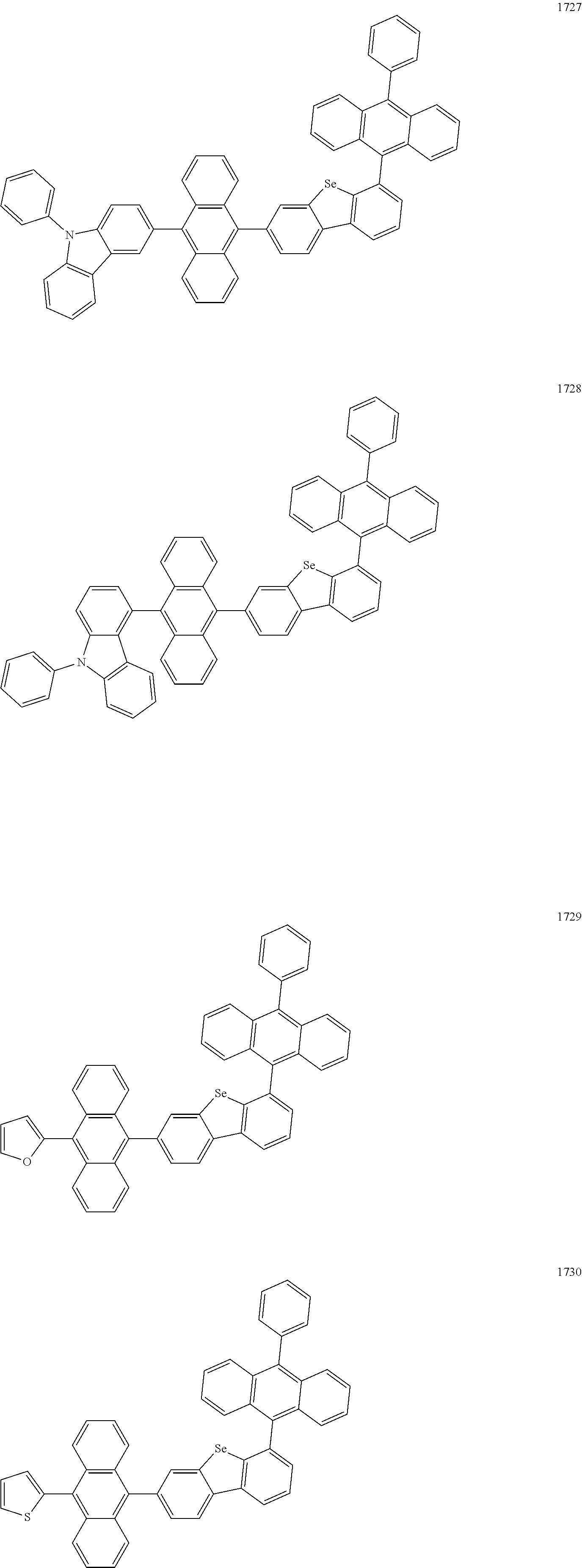 [SMILES: c1ccc(-c2c3ccccc3c(-c3cccc4c3[se]c3cc(-c5c6ccccc6c(-c6ccc7c(c6)c6ccccc6n7-c6ccccc6)c6ccccc56)ccc34)c3ccccc23)cc1 ; c1ccc(-c2c3ccccc3c(-c3cccc4c3[se]c3cc(-c5c6ccccc6c(-c6cccc7c6c6ccccc6n7-c6ccccc6)c6ccccc56)ccc34)c3ccccc23)cc1 ; c1ccc(-c2c3ccccc3c(-c3cccc4c3[se]c3cc(-c5c6ccccc6c(-c6ccco6)c6ccccc56)ccc34)c3ccccc23)cc1 ; c1ccc(-c2c3ccccc3c(-c3cccc4c3[se]c3cc(-c5c6ccccc6c(-c6cccs6)c6ccccc56)ccc34)c3ccccc23)cc1]

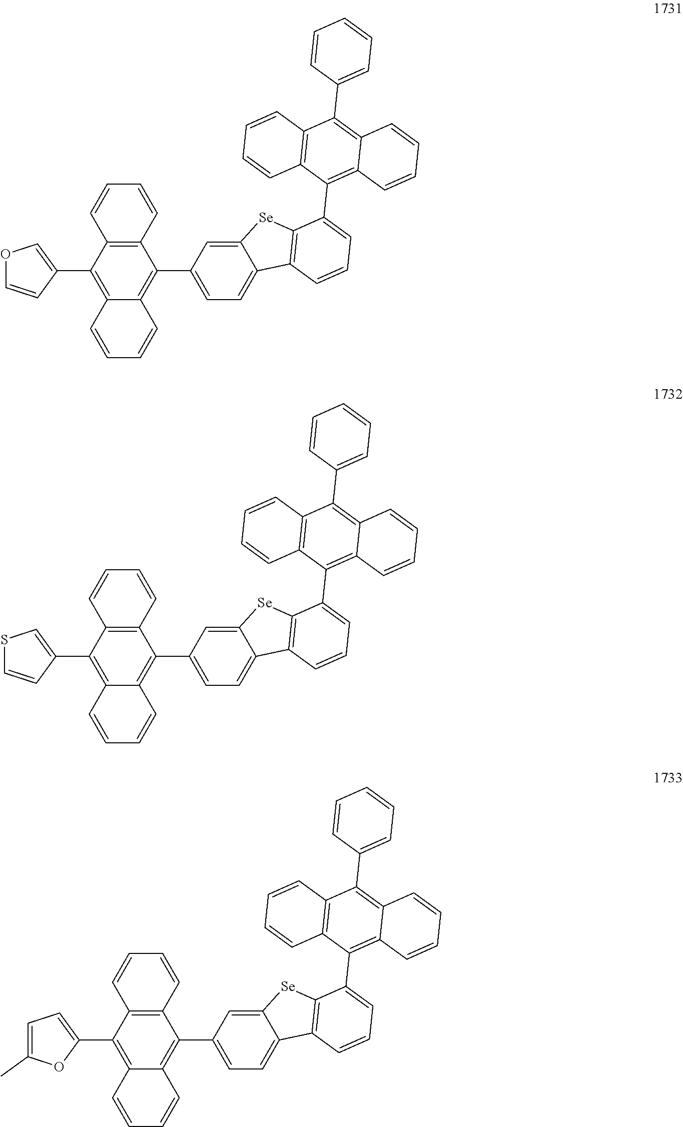 [SMILES: c1ccc(-c2c3ccccc3c(-c3cccc4c3[se]c3cc(-c5c6ccccc6c(-c6ccoc6)c6ccccc56)ccc34)c3ccccc23)cc1 ; c1ccc(-c2c3ccccc3c(-c3cccc4c3[se]c3cc(-c5c6ccccc6c(-c6ccsc6)c6ccccc56)ccc34)c3ccccc23)cc1 ; Cc1ccc(-c2c3ccccc3c(-c3ccc4c(c3)[se]c3c(-c5c6ccccc6c(-c6ccccc6)c6ccccc56)cccc34)c3ccccc23)o1]

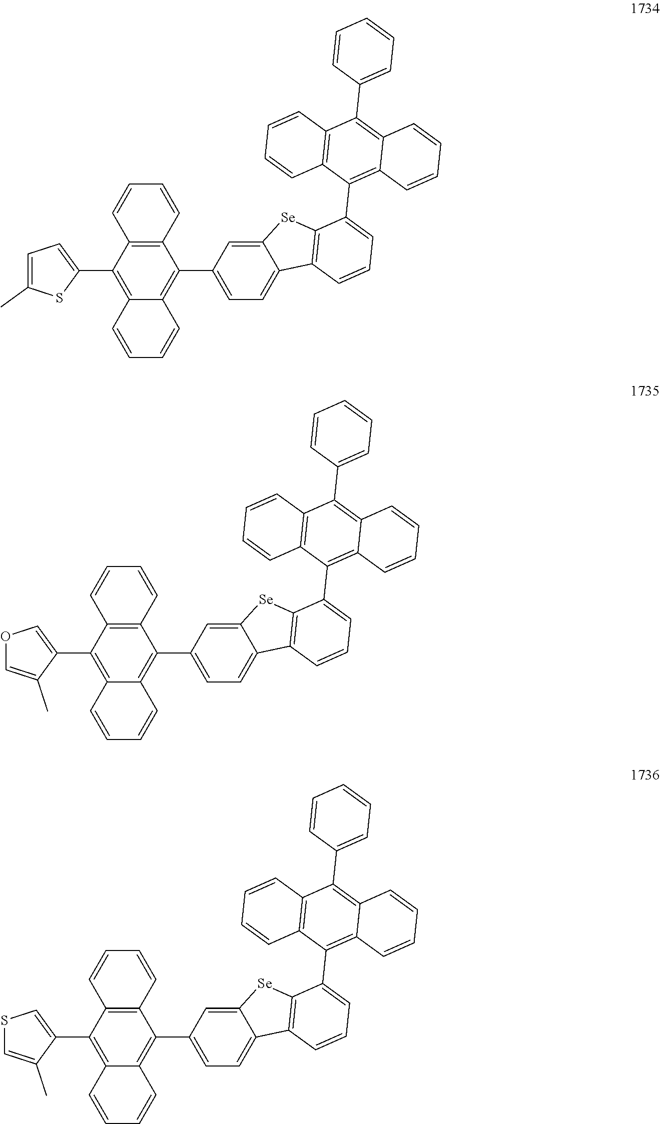 [SMILES: Cc1ccc(-c2c3ccccc3c(-c3ccc4c(c3)[se]c3c(-c5c6ccccc6c(-c6ccccc6)c6ccccc56)cccc34)c3ccccc23)s1 ; Cc1cocc1-c1c2ccccc2c(-c2ccc3c(c2)[se]c2c(-c4c5ccccc5c(-c5ccccc5)c5ccccc45)cccc23)c2ccccc12 ; Cc1cscc1-c1c2ccccc2c(-c2ccc3c(c2)[se]c2c(-c4c5ccccc5c(-c5ccccc5)c5ccccc45)cccc23)c2ccccc12]

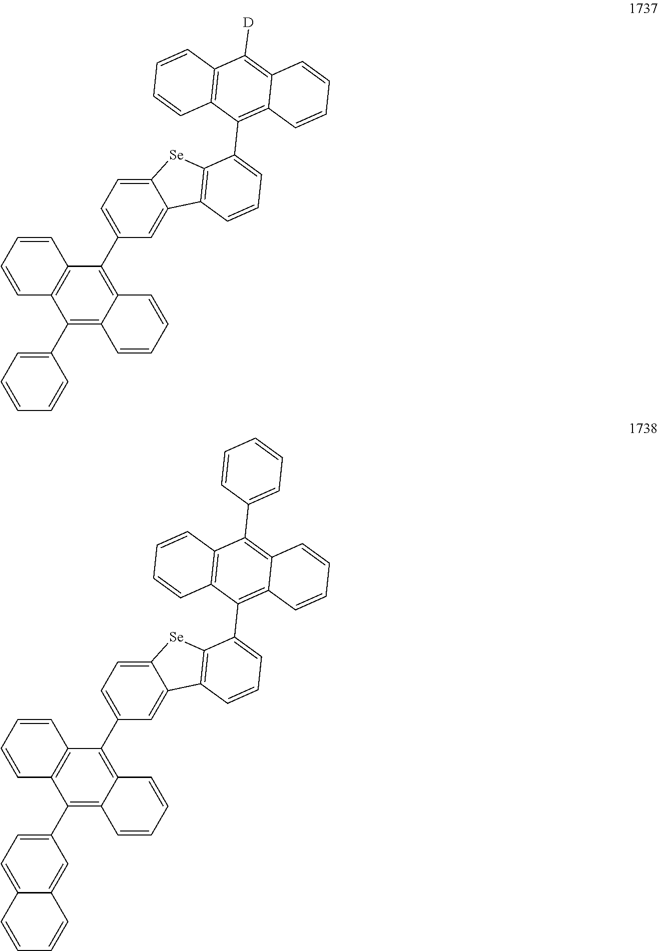 [SMILES: [2H]c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3cccc4c3[se]c3ccc(-c5c6ccccc6c(-c6ccc7ccccc7c6)c6ccccc56)cc34)c3ccccc23)cc1]

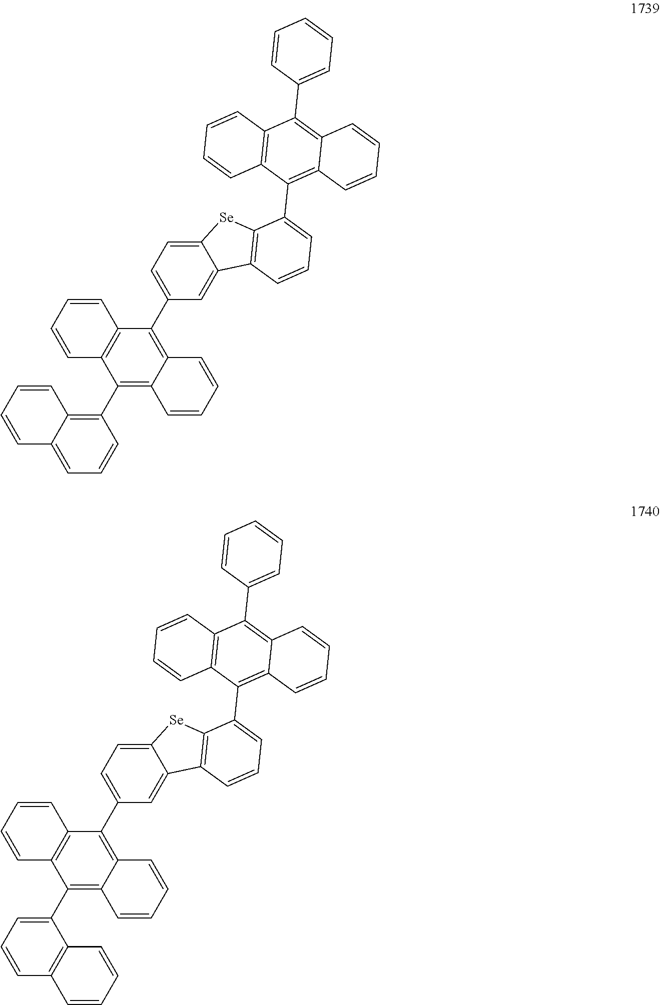 [SMILES: c1ccc(-c2c3ccccc3c(-c3cccc4c3[se]c3ccc(-c5c6ccccc6c(-c6cccc7ccccc67)c6ccccc56)cc34)c3ccccc23)cc1 ; c1ccc(-c2c3ccccc3c(-c3cccc4c3[se]c3ccc(-c5c6ccccc6c(-c6cccc7ccccc67)c6ccccc56)cc34)c3ccccc23)cc1]

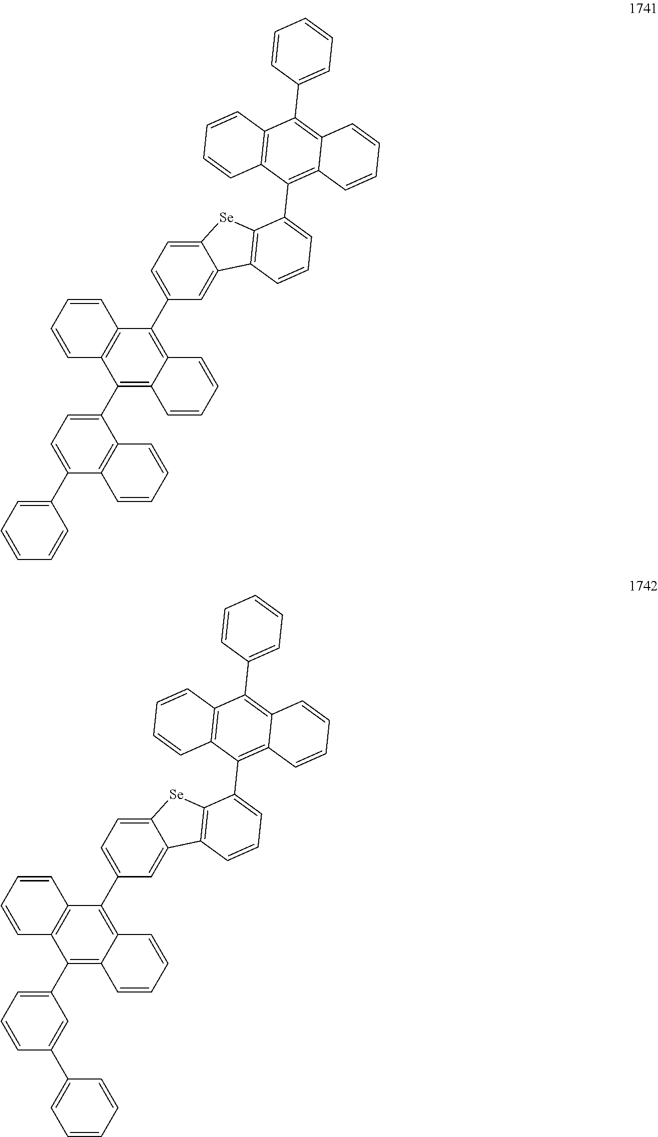 [SMILES: c1ccc(-c2ccc(-c3c4ccccc4c(-c4ccc5[se]c6c(-c7c8ccccc8c(-c8ccccc8)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8ccccc8)c8ccccc78)cccc6c5c4)c4ccccc34)c2)cc1]

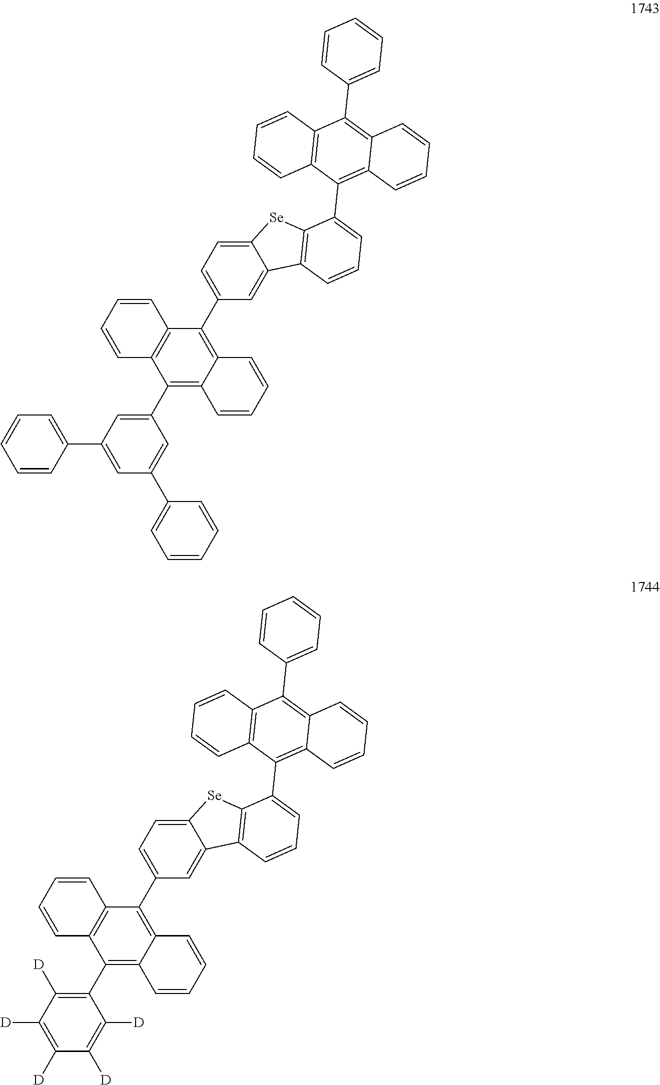 [SMILES: c1ccc(-c2cc(-c3ccccc3)cc(-c3c4ccccc4c(-c4ccc5[se]c6c(-c7c8ccccc8c(-c8ccccc8)c8ccccc78)cccc6c5c4)c4ccccc34)c2)cc1 ; [2H]c1c([2H])c([2H])c(-c2c3ccccc3c(-c3ccc4[se]c5c(-c6c7ccccc7c(-c7ccccc7)c7ccccc67)cccc5c4c3)c3ccccc23)c([2H])c1[2H]]

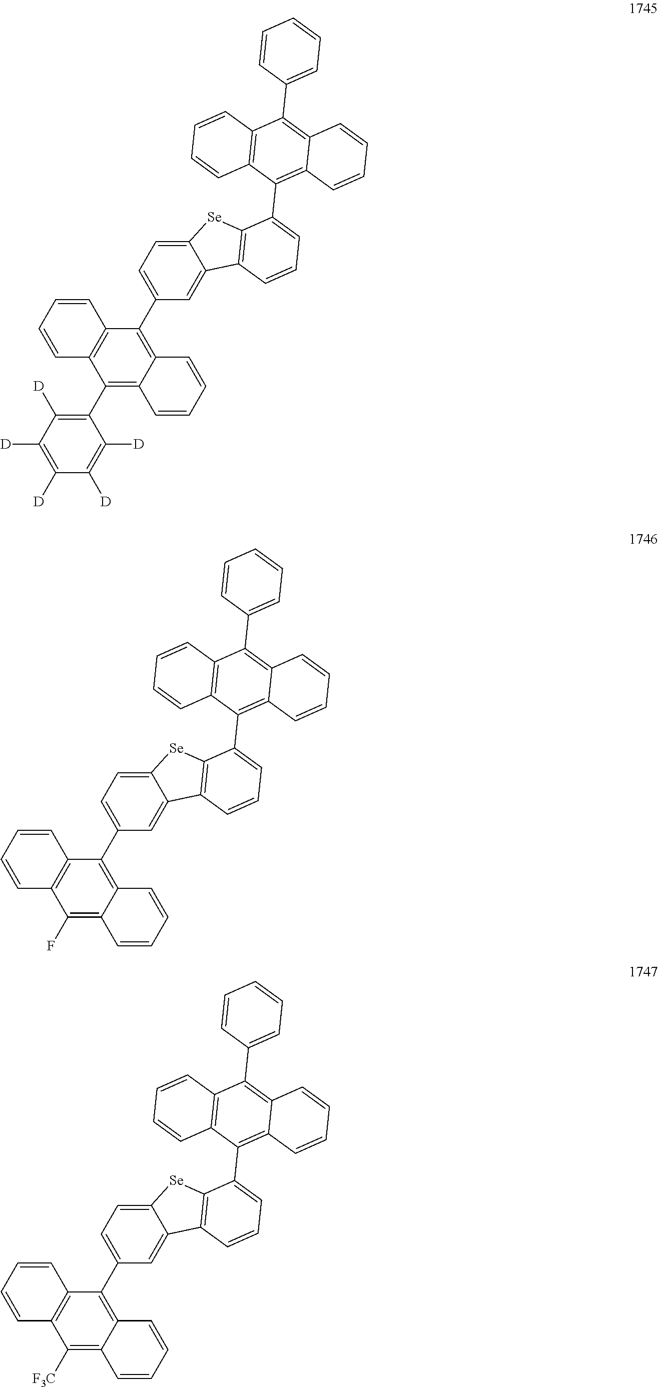 [SMILES: [2H]c1c([2H])c([2H])c(-c2c3ccccc3c(-c3ccc4[se]c5c(-c6c7ccccc7c(-c7ccccc7)c7ccccc67)cccc5c4c3)c3ccccc23)c([2H])c1[2H] ; Fc1c2ccccc2c(-c2ccc3[se]c4c(-c5c6ccccc6c(-c6ccccc6)c6ccccc56)cccc4c3c2)c2ccccc12 ; FC(F)(F)c1c2ccccc2c(-c2ccc3[se]c4c(-c5c6ccccc6c(-c6ccccc6)c6ccccc56)cccc4c3c2)c2ccccc12]

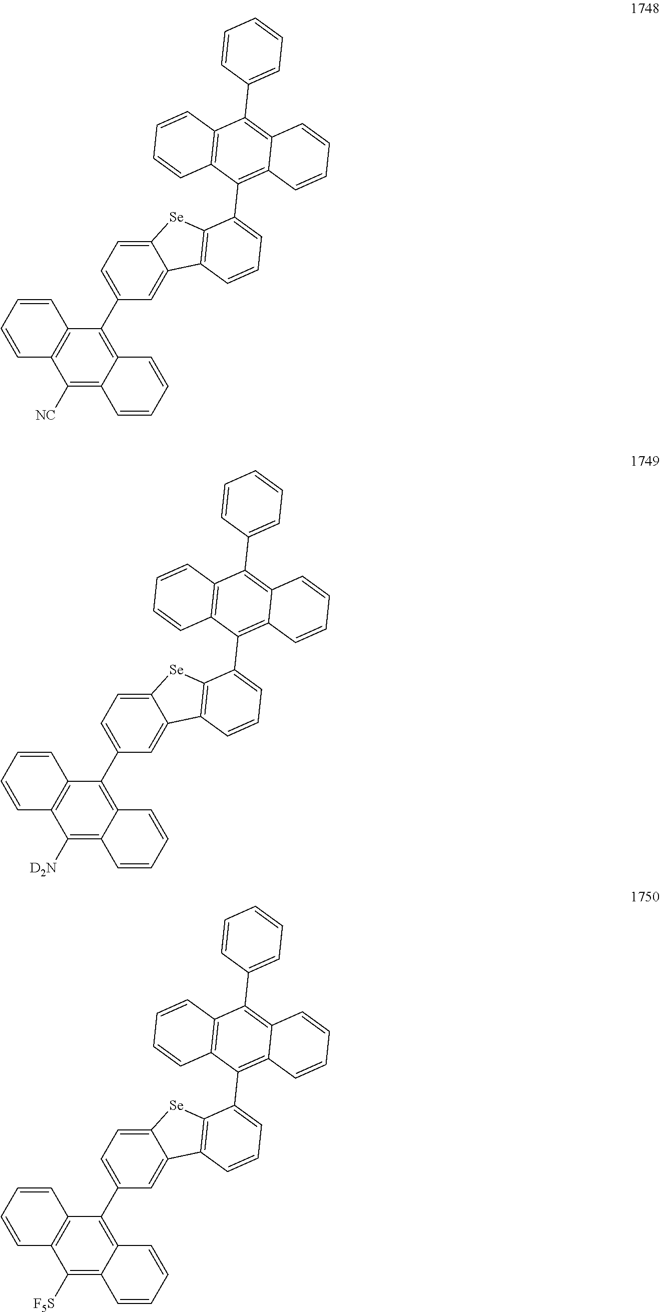

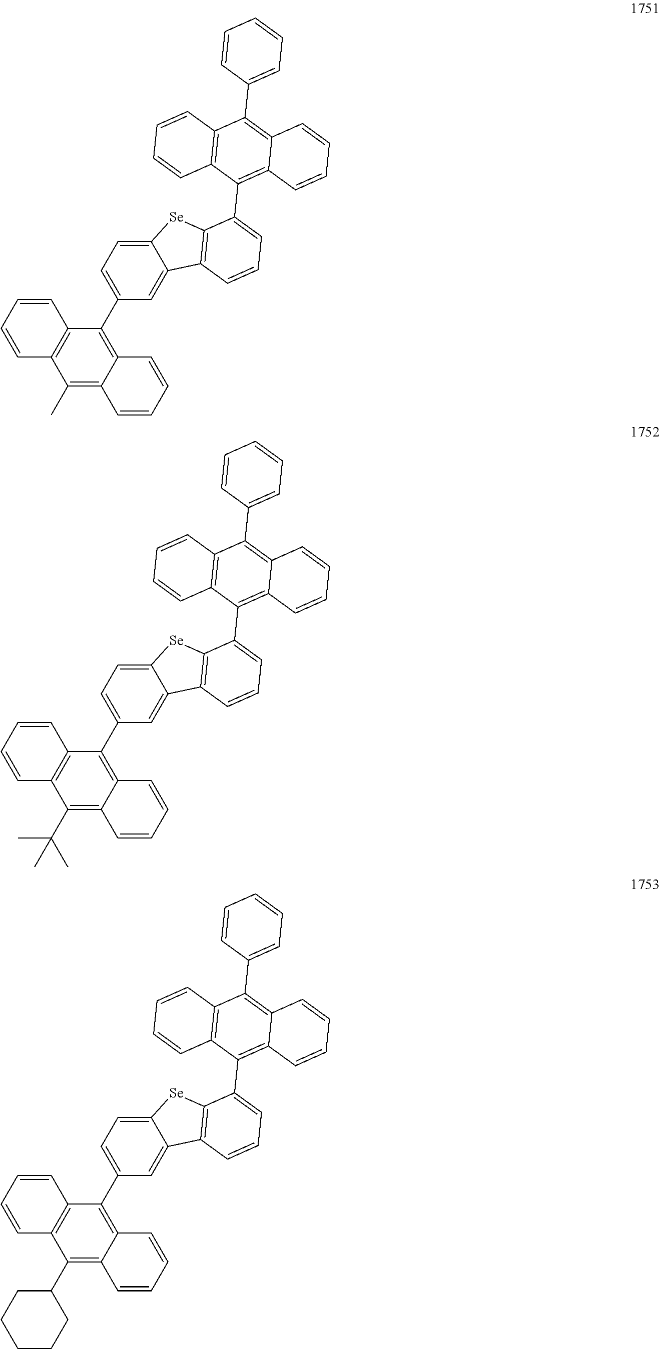 [SMILES: Cc1c2ccccc2c(-c2ccc3[se]c4c(-c5c6ccccc6c(-c6ccccc6)c6ccccc56)cccc4c3c2)c2ccccc12 ; CC(C)(C)c1c2ccccc2c(-c2ccc3[se]c4c(-c5c6ccccc6c(-c6ccccc6)c6ccccc56)cccc4c3c2)c2ccccc12 ; c1ccc(-c2c3ccccc3c(-c3cccc4c3[se]c3ccc(-c5c6ccccc6c(C6CCCCC6)c6ccccc56)cc34)c3ccccc23)cc1]

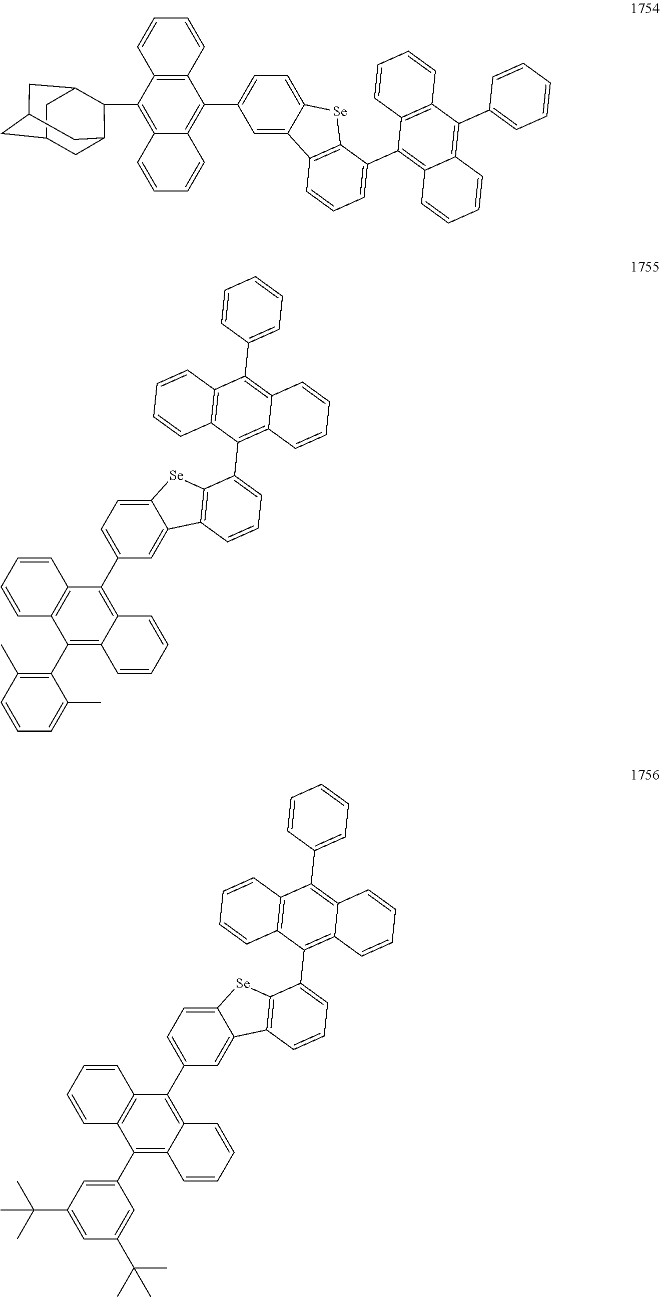 [SMILES: c1ccc(-c2c3ccccc3c(-c3cccc4c3[se]c3ccc(-c5c6ccccc6c(C6C7CC8CC(C7)CC6C8)c6ccccc56)cc34)c3ccccc23)cc1 ; Cc1cccc(C)c1-c1c2ccccc2c(-c2ccc3[se]c4c(-c5c6ccccc6c(-c6ccccc6)c6ccccc56)cccc4c3c2)c2ccccc12 ; CC(C)(C)c1cc(-c2c3ccccc3c(-c3ccc4[se]c5c(-c6c7ccccc7c(-c7ccccc7)c7ccccc67)cccc5c4c3)c3ccccc23)cc(C(C)(C)C)c1]

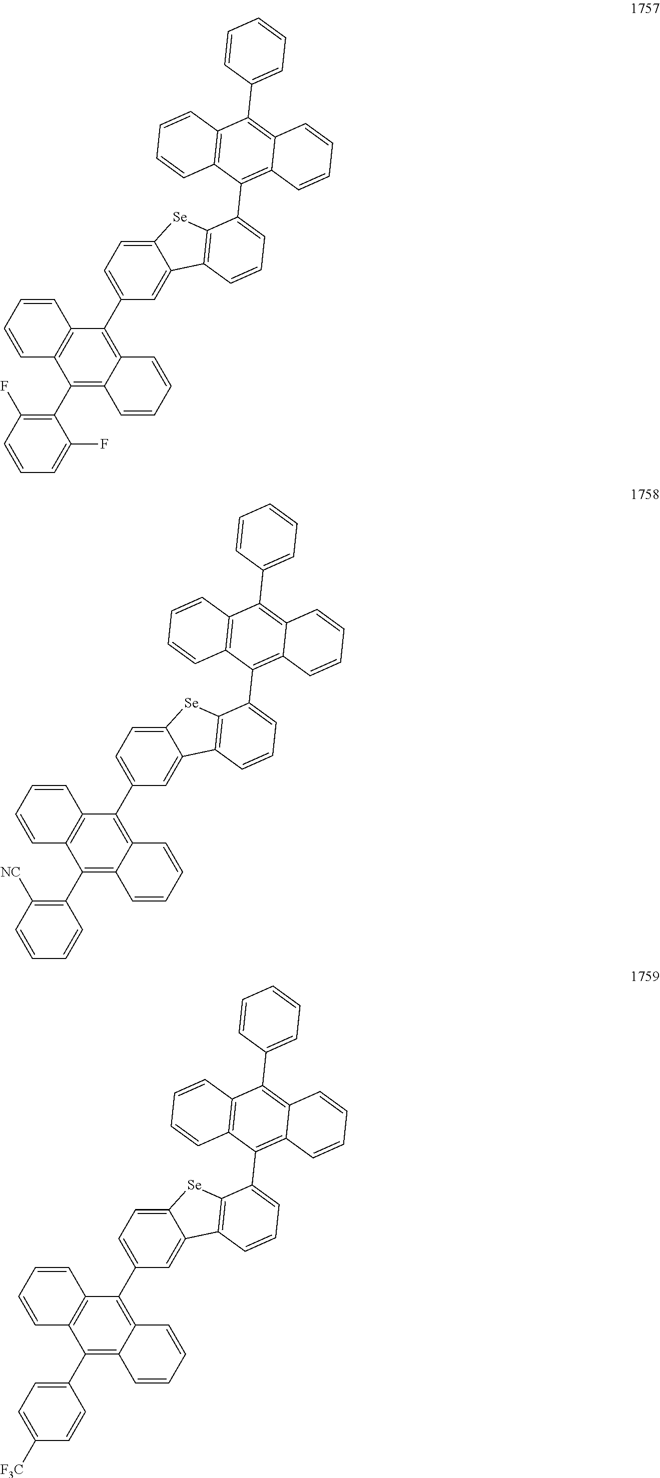 [SMILES: Fc1cccc(F)c1-c1c2ccccc2c(-c2ccc3[se]c4c(-c5c6ccccc6c(-c6ccccc6)c6ccccc56)cccc4c3c2)c2ccccc12 ; N#Cc1ccccc1-c1c2ccccc2c(-c2ccc3[se]c4c(-c5c6ccccc6c(-c6ccccc6)c6ccccc56)cccc4c3c2)c2ccccc12 ; FC(F)(F)c1ccc(-c2c3ccccc3c(-c3ccc4[se]c5c(-c6c7ccccc7c(-c7ccccc7)c7ccccc67)cccc5c4c3)c3ccccc23)cc1]

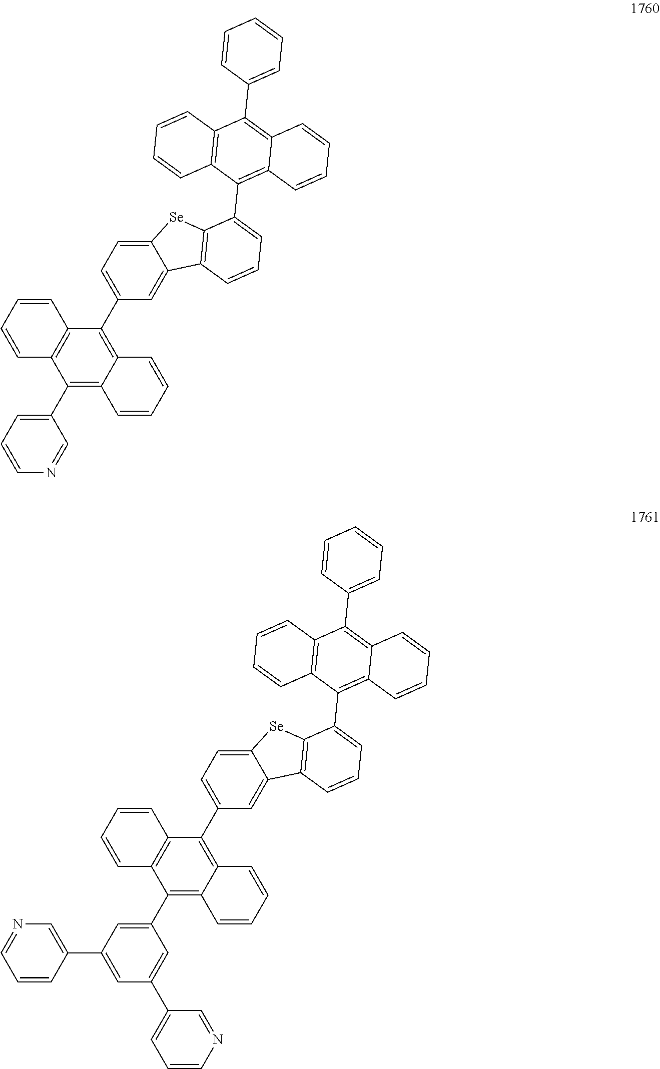 [SMILES: c1ccc(-c2c3ccccc3c(-c3cccc4c3[se]c3ccc(-c5c6ccccc6c(-c6cccnc6)c6ccccc56)cc34)c3ccccc23)cc1 ; c1ccc(-c2c3ccccc3c(-c3cccc4c3[se]c3ccc(-c5c6ccccc6c(-c6cc(-c7cccnc7)cc(-c7cccnc7)c6)c6ccccc56)cc34)c3ccccc23)cc1]

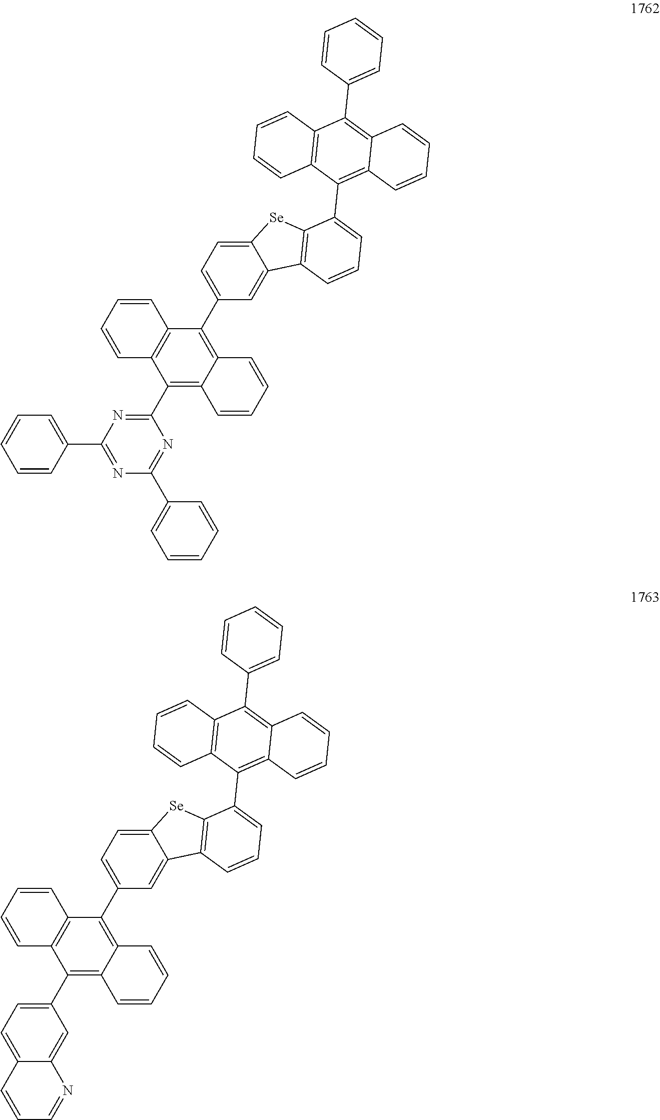 [SMILES: c1ccc(-c2nc(-c3ccccc3)nc(-c3c4ccccc4c(-c4ccc5[se]c6c(-c7c8ccccc8c(-c8ccccc8)c8ccccc78)cccc6c5c4)c4ccccc34)n2)cc1 ; c1ccc(-c2c3ccccc3c(-c3cccc4c3[se]c3ccc(-c5c6ccccc6c(-c6ccc7cccnc7c6)c6ccccc56)cc34)c3ccccc23)cc1]

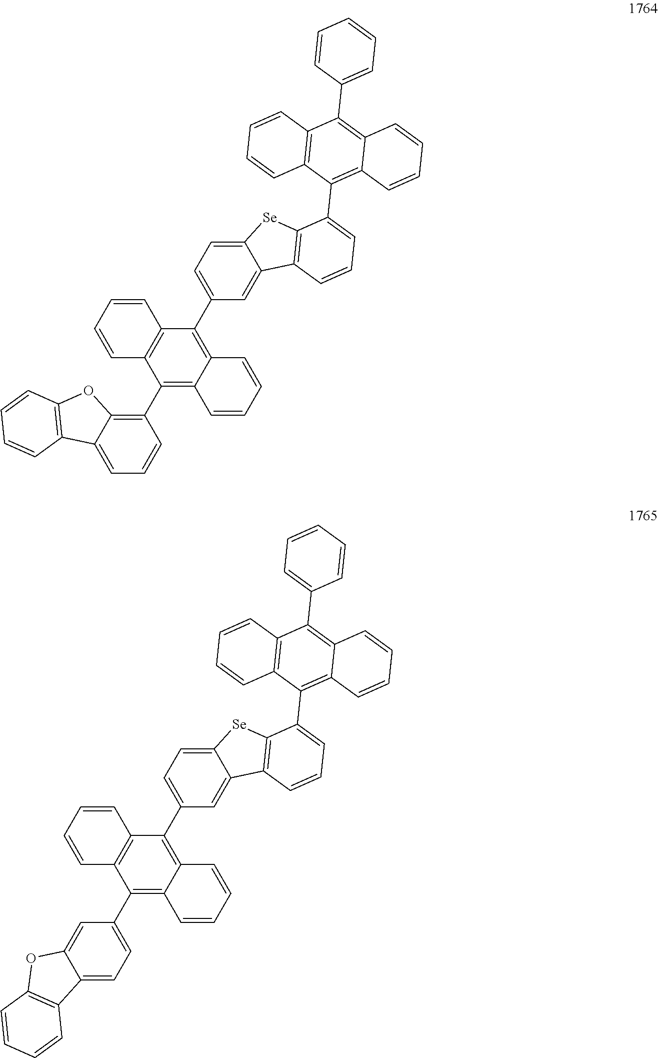 [SMILES: c1ccc(-c2c3ccccc3c(-c3cccc4c3[se]c3ccc(-c5c6ccccc6c(-c6cccc7c6oc6ccccc67)c6ccccc56)cc34)c3ccccc23)cc1 ; c1ccc(-c2c3ccccc3c(-c3cccc4c3[se]c3ccc(-c5c6ccccc6c(-c6ccc7c(c6)oc6ccccc67)c6ccccc56)cc34)c3ccccc23)cc1]

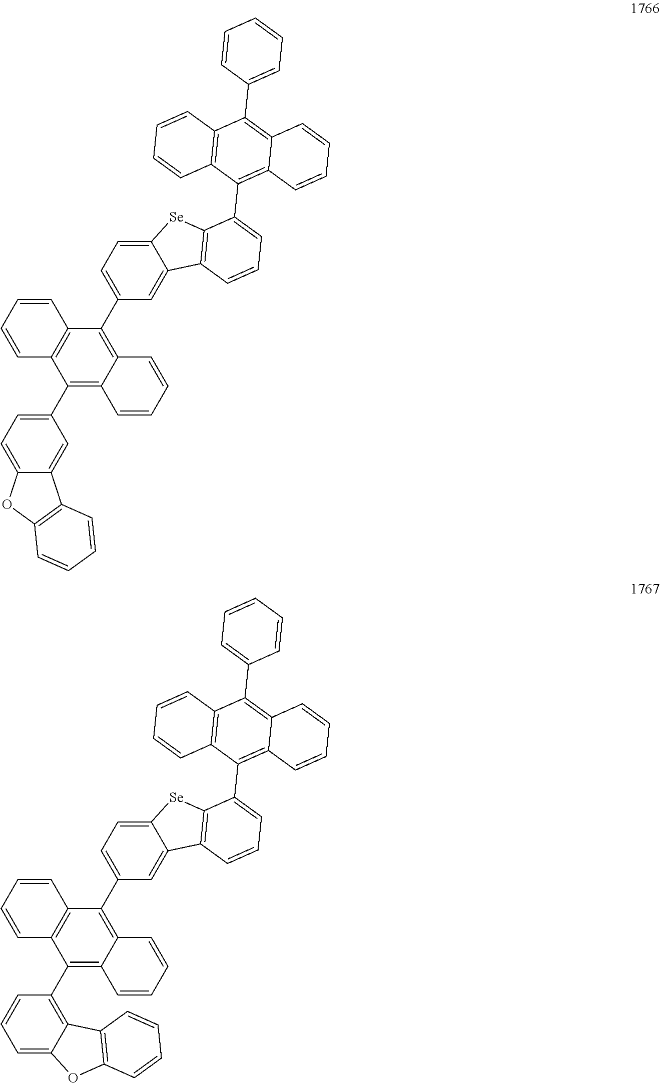 [SMILES: c1ccc(-c2c3ccccc3c(-c3cccc4c3[se]c3ccc(-c5c6ccccc6c(-c6ccc7oc8ccccc8c7c6)c6ccccc56)cc34)c3ccccc23)cc1 ; c1ccc(-c2c3ccccc3c(-c3cccc4c3[se]c3ccc(-c5c6ccccc6c(-c6cccc7oc8ccccc8c67)c6ccccc56)cc34)c3ccccc23)cc1]

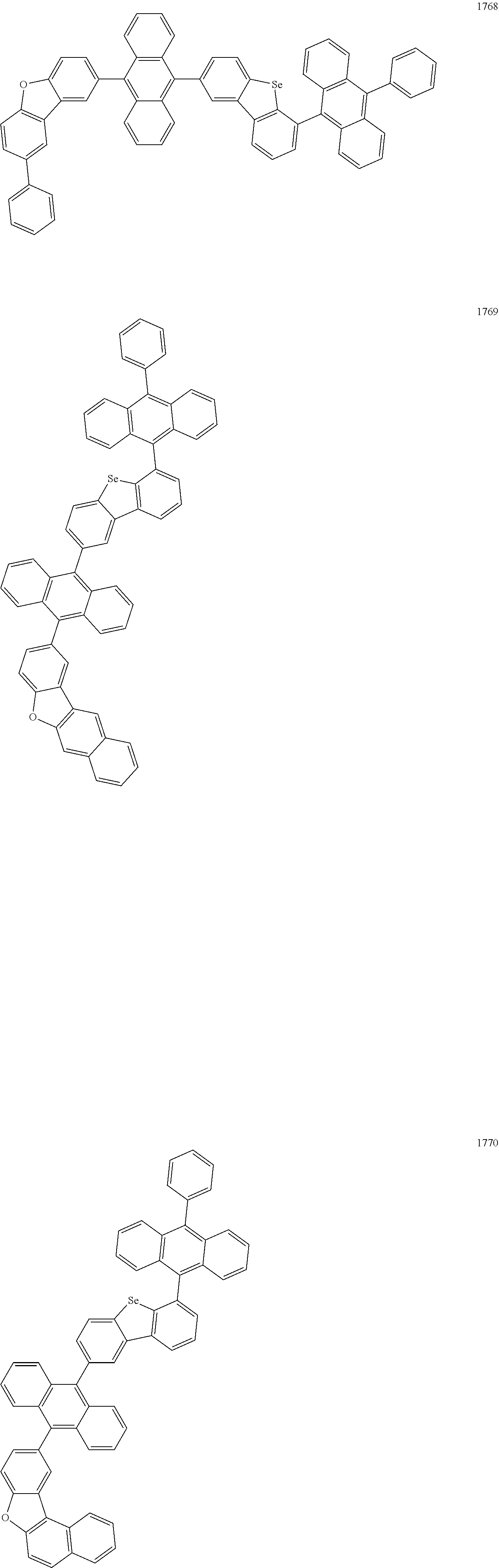 [SMILES: c1ccc(-c2ccc3oc4ccc(-c5c6ccccc6c(-c6ccc7[se]c8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3[se]c3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3cccc4c3[se]c3ccc(-c5c6ccccc6c(-c6ccc7oc8ccc9ccccc9c8c7c6)c6ccccc56)cc34)c3ccccc23)cc1]

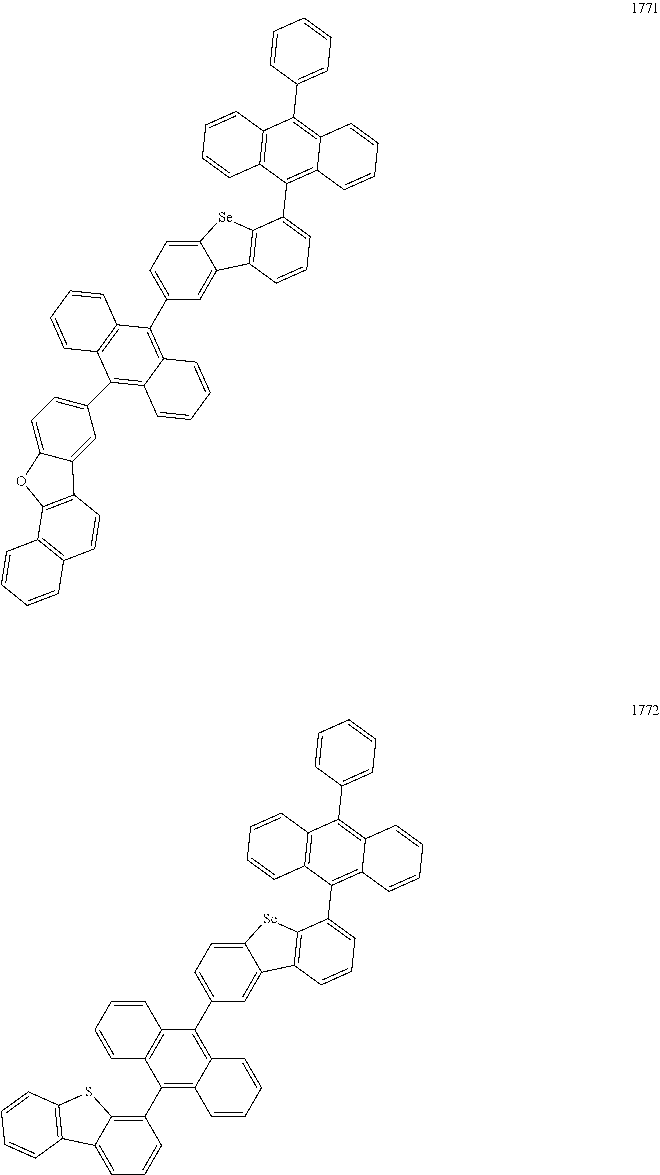 [SMILES: c1ccc(-c2c3ccccc3c(-c3cccc4c3[se]c3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3cccc4c3[se]c3ccc(-c5c6ccccc6c(-c6cccc7c6sc6ccccc67)c6ccccc56)cc34)c3ccccc23)cc1]

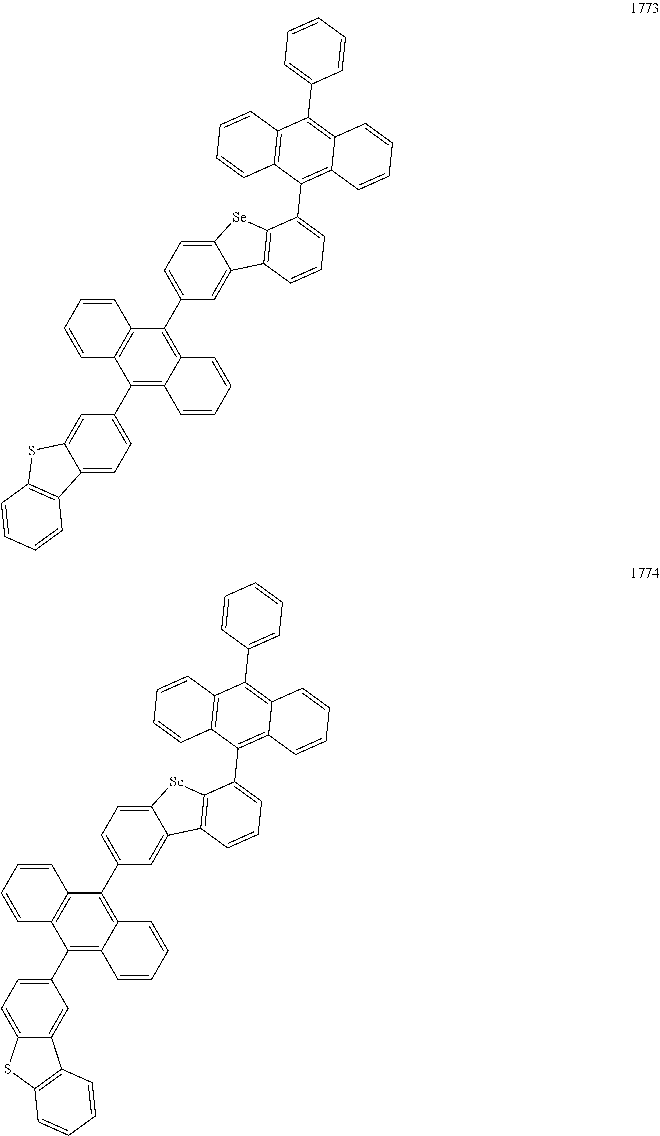 [SMILES: c1ccc(-c2c3ccccc3c(-c3cccc4c3[se]c3ccc(-c5c6ccccc6c(-c6ccc7c(c6)sc6ccccc67)c6ccccc56)cc34)c3ccccc23)cc1 ; c1ccc(-c2c3ccccc3c(-c3cccc4c3[se]c3ccc(-c5c6ccccc6c(-c6ccc7sc8ccccc8c7c6)c6ccccc56)cc34)c3ccccc23)cc1]

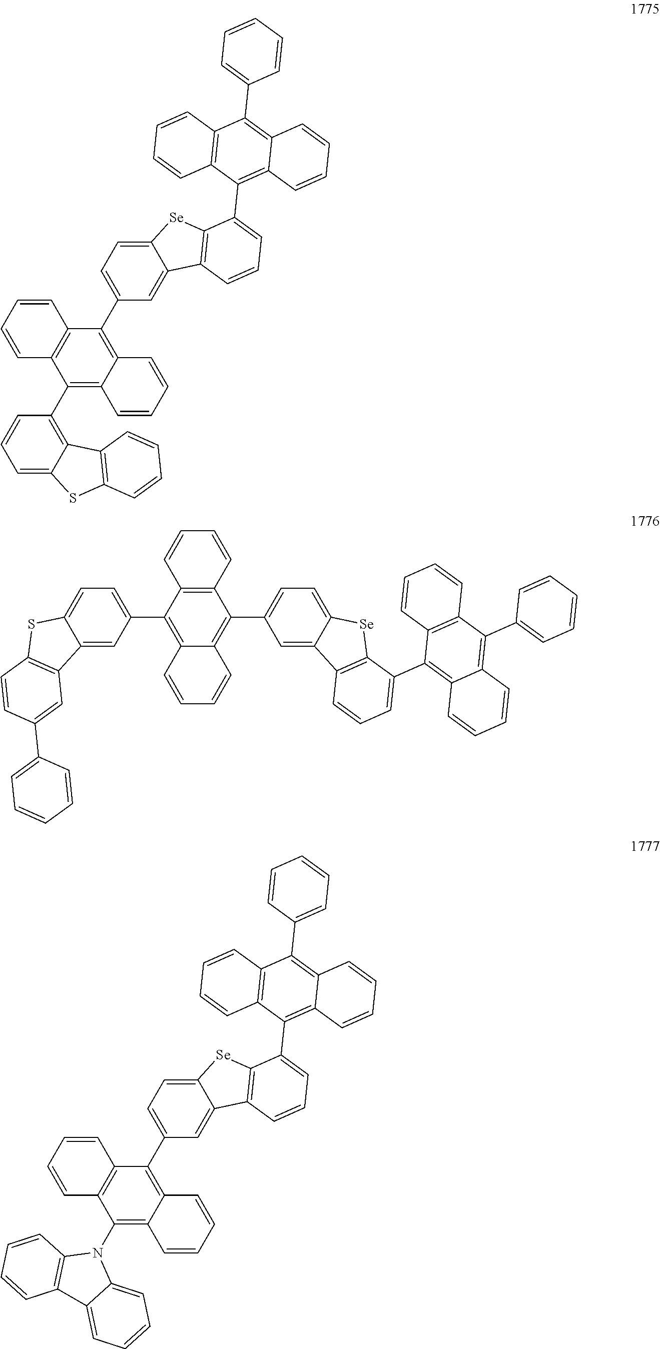 [SMILES: c1ccc(-c2c3ccccc3c(-c3cccc4c3[se]c3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7[se]c8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3[se]c3ccc(-c5c6ccccc6c(-n6c7ccccc7c7ccccc76)c6ccccc56)cc34)c3ccccc23)cc1]

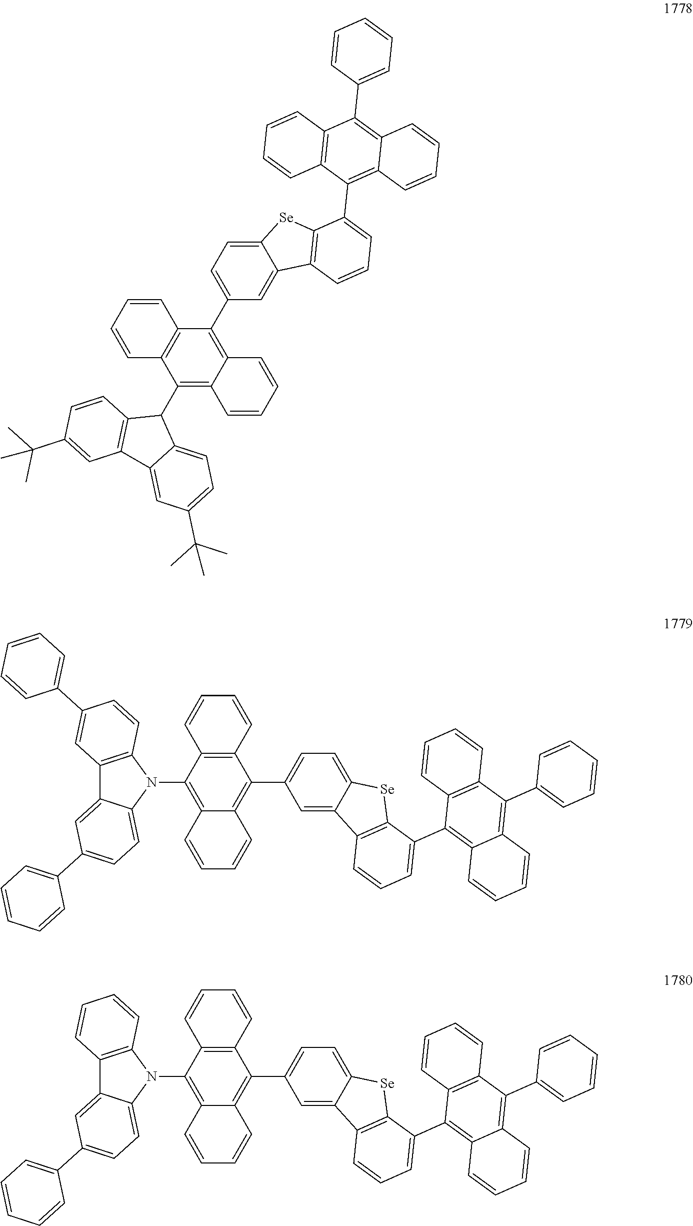 [SMILES: CC(C)(C)c1ccc2c(c1)-c1cc(C(C)(C)C)ccc1C2c1c2ccccc2c(-c2ccc3[se]c4c(-c5c6ccccc6c(-c6ccccc6)c6ccccc56)cccc4c3c2)c2ccccc12 ; c1ccc(-c2ccc3c(c2)c2cc(-c4ccccc4)ccc2n3-c2c3ccccc3c(-c3ccc4[se]c5c(-c6c7ccccc7c(-c7ccccc7)c7ccccc67)cccc5c4c3)c3ccccc23)cc1 ; c1ccc(-c2ccc3c(c2)c2ccccc2n3-c2c3ccccc3c(-c3ccc4[se]c5c(-c6c7ccccc7c(-c7ccccc7)c7ccccc67)cccc5c4c3)c3ccccc23)cc1]

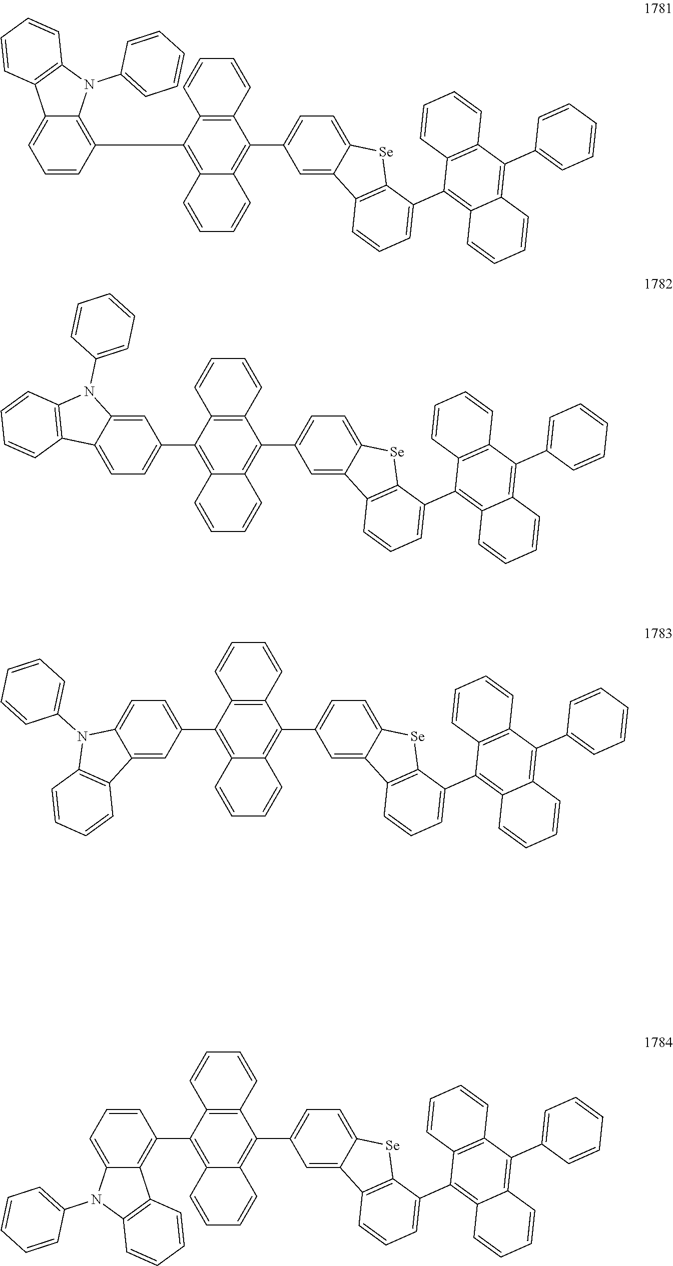 [SMILES: c1ccc(-c2c3ccccc3c(-c3cccc4c3[se]c3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3cccc4c3[se]c3ccc(-c5c6ccccc6c(-c6ccc7c8ccccc8n(-c8ccccc8)c7c6)c6ccccc56)cc34)c3ccccc23)cc1 ; c1ccc(-c2c3ccccc3c(-c3cccc4c3[se]c3ccc(-c5c6ccccc6c(-c6ccc7c(c6)c6ccccc6n7-c6ccccc6)c6ccccc56)cc34)c3ccccc23)cc1 ; c1ccc(-c2c3ccccc3c(-c3cccc4c3[se]c3ccc(-c5c6ccccc6c(-c6cccc7c6c6ccccc6n7-c6ccccc6)c6ccccc56)cc34)c3ccccc23)cc1]

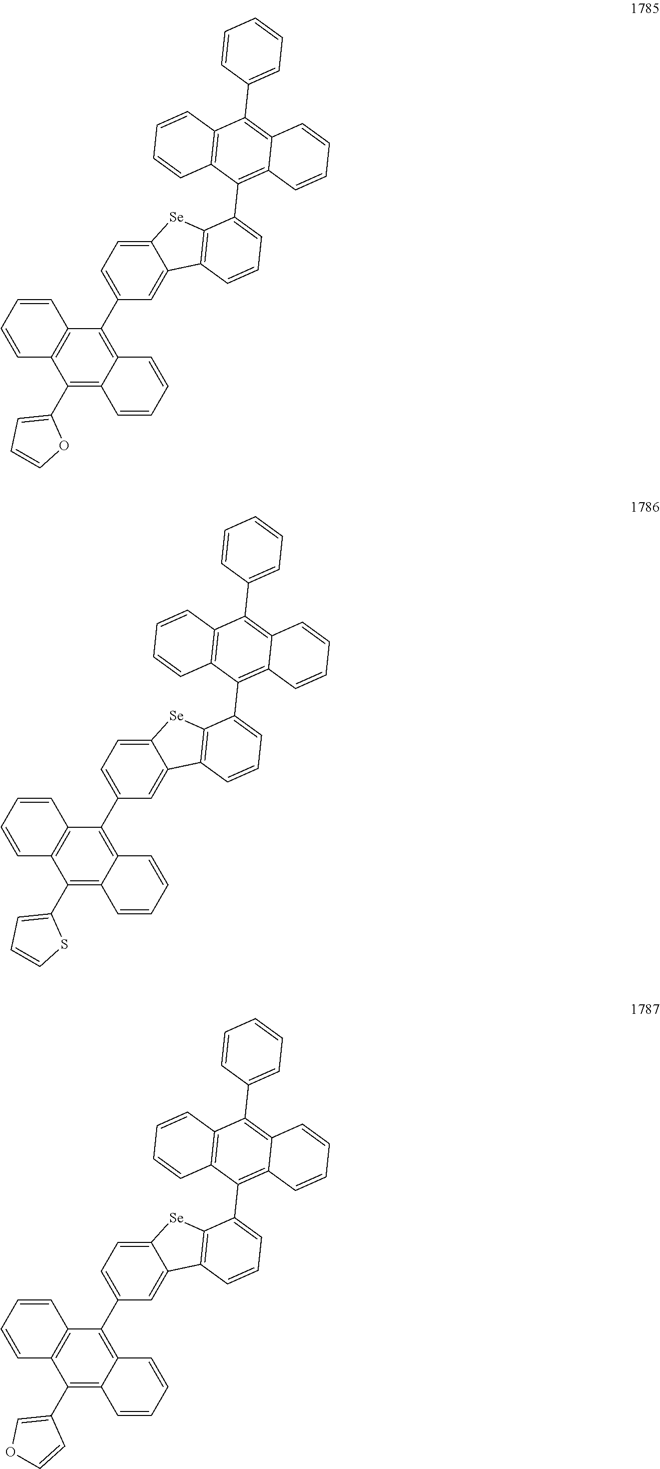 [SMILES: c1ccc(-c2c3ccccc3c(-c3cccc4c3[se]c3ccc(-c5c6ccccc6c(-c6ccco6)c6ccccc56)cc34)c3ccccc23)cc1 ; c1ccc(-c2c3ccccc3c(-c3cccc4c3[se]c3ccc(-c5c6ccccc6c(-c6cccs6)c6ccccc56)cc34)c3ccccc23)cc1 ; c1ccc(-c2c3ccccc3c(-c3cccc4c3[se]c3ccc(-c5c6ccccc6c(-c6ccoc6)c6ccccc56)cc34)c3ccccc23)cc1]

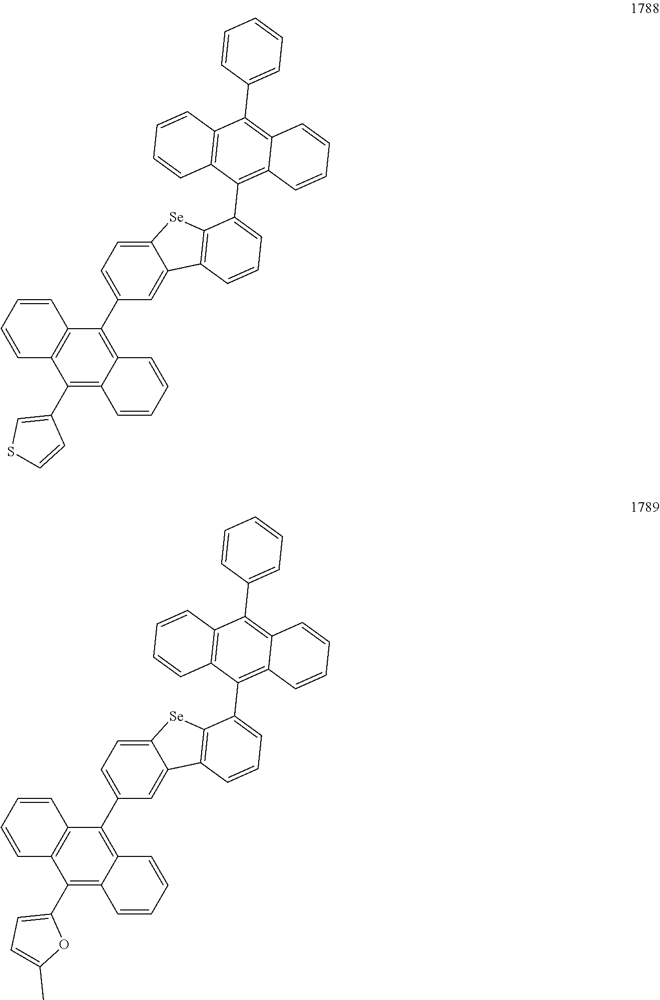 [SMILES: c1ccc(-c2c3ccccc3c(-c3cccc4c3[se]c3ccc(-c5c6ccccc6c(-c6ccsc6)c6ccccc56)cc34)c3ccccc23)cc1 ; Cc1ccc(-c2c3ccccc3c(-c3ccc4[se]c5c(-c6c7ccccc7c(-c7ccccc7)c7ccccc67)cccc5c4c3)c3ccccc23)o1]

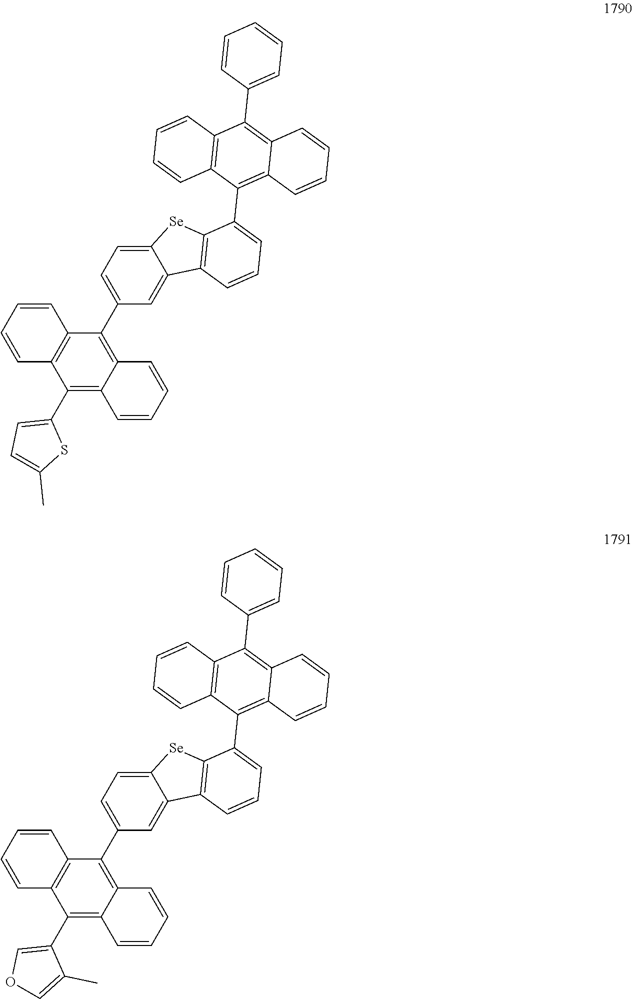 [SMILES: Cc1ccc(-c2c3ccccc3c(-c3ccc4[se]c5c(-c6c7ccccc7c(-c7ccccc7)c7ccccc67)cccc5c4c3)c3ccccc23)s1 ; Cc1cocc1-c1c2ccccc2c(-c2ccc3[se]c4c(-c5c6ccccc6c(-c6ccccc6)c6ccccc56)cccc4c3c2)c2ccccc12]

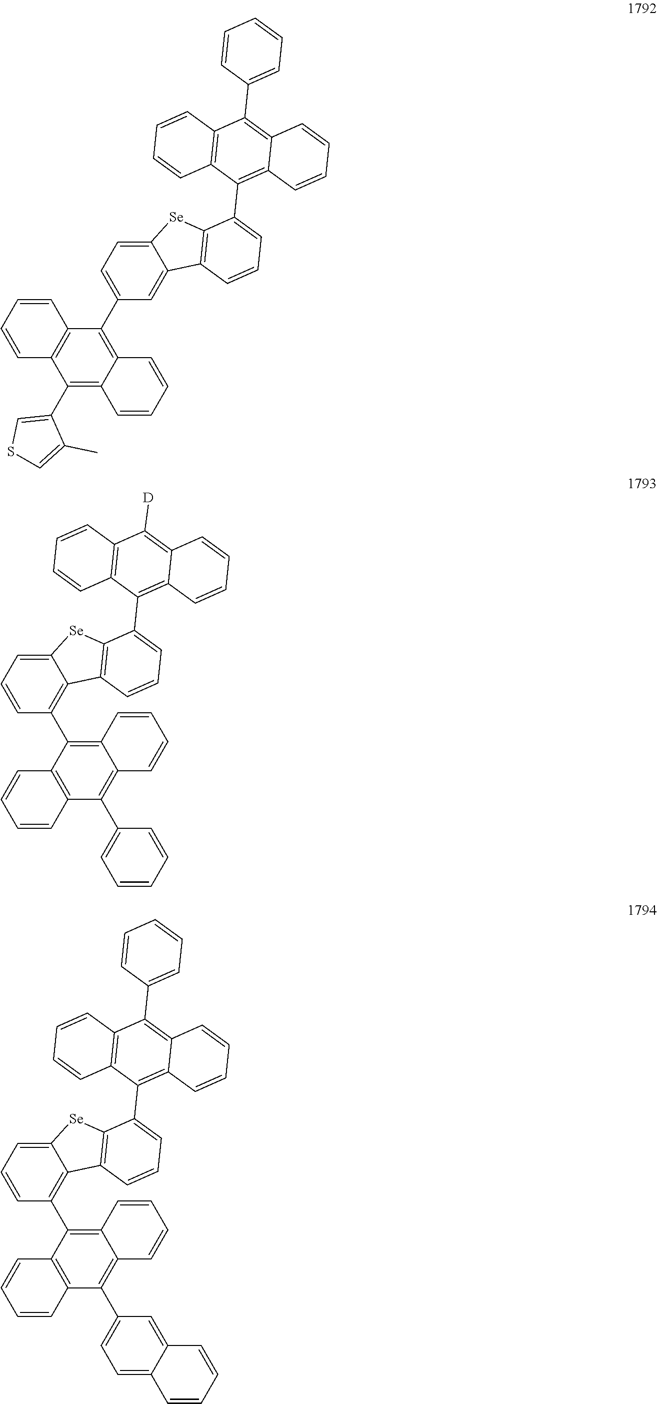

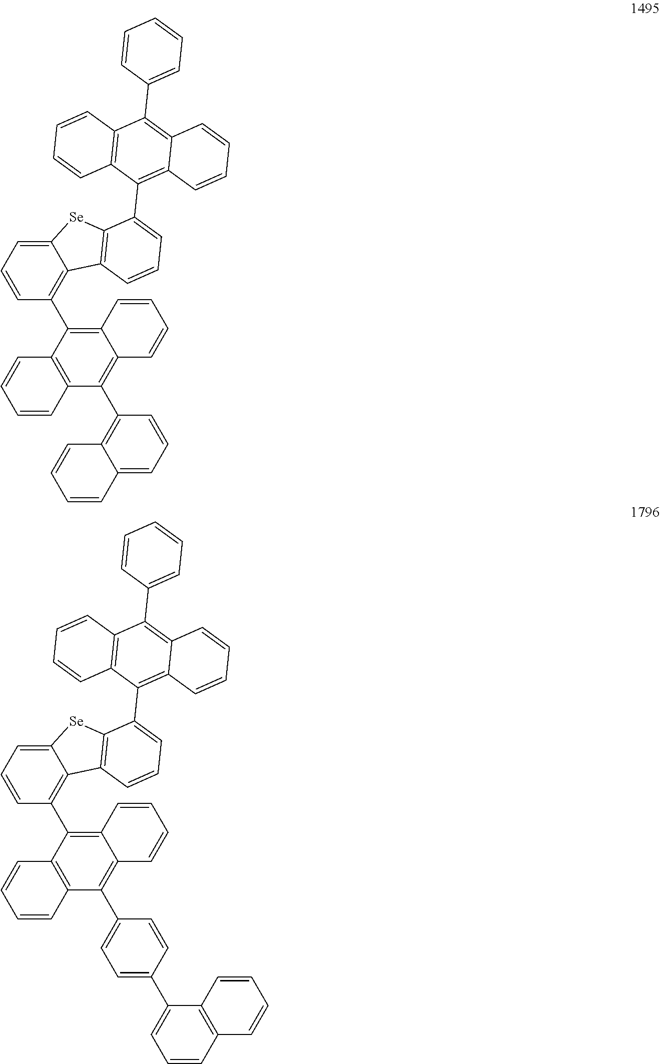

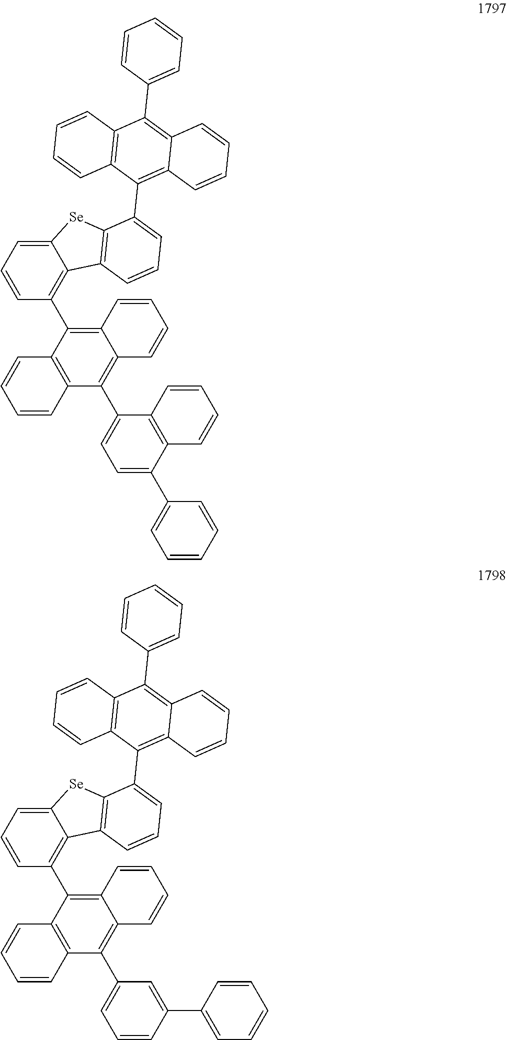

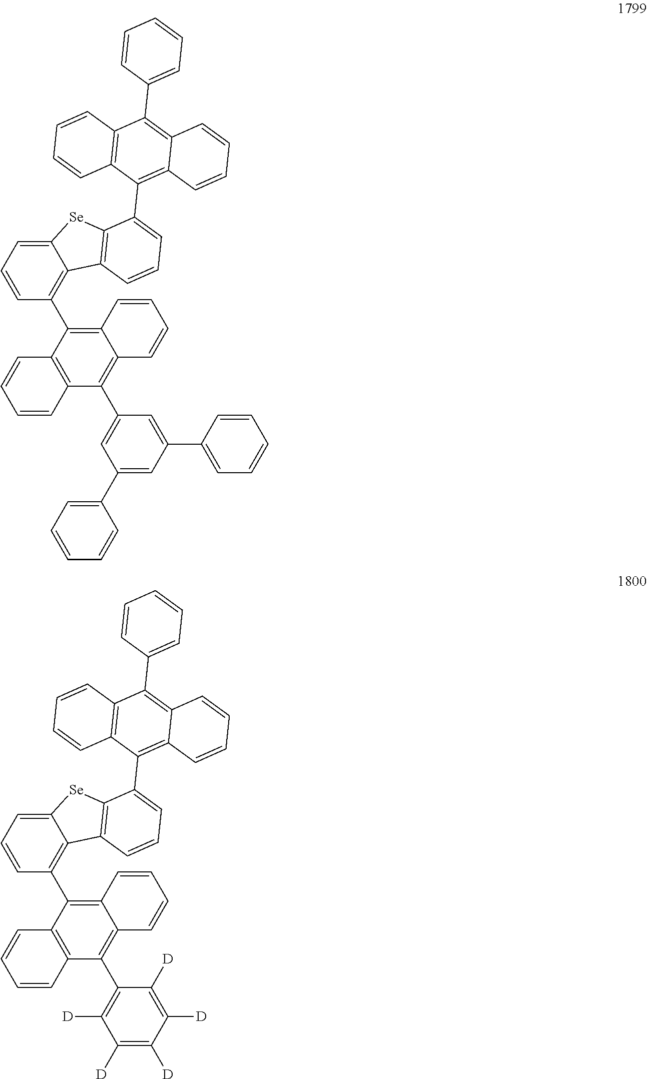 [SMILES: c1ccc(-c2cc(-c3ccccc3)cc(-c3c4ccccc4c(-c4cccc5[se]c6c(-c7c8ccccc8c(-c8ccccc8)c8ccccc78)cccc6c45)c4ccccc34)c2)cc1 ; [2H]c1c([2H])c([2H])c(-c2c3ccccc3c(-c3cccc4[se]c5c(-c6c7ccccc7c(-c7ccccc7)c7ccccc67)cccc5c34)c3ccccc23)c([2H])c1[2H]]

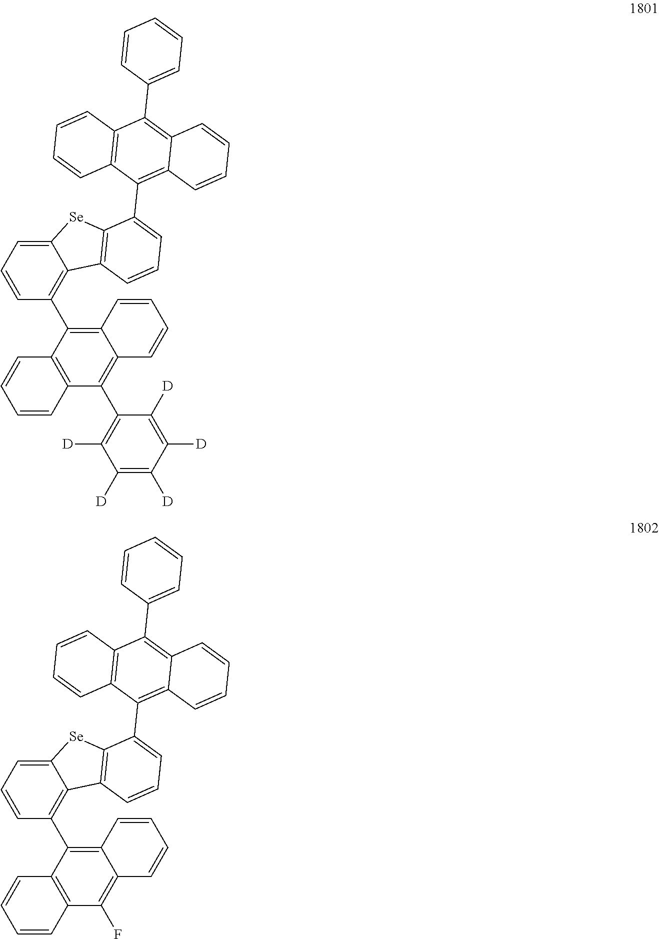

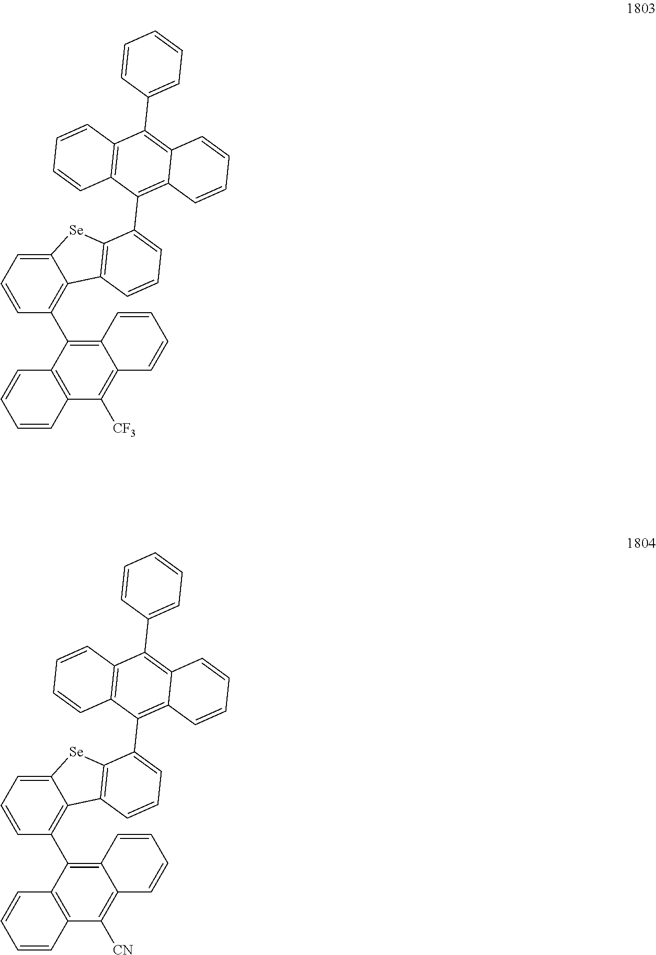 [SMILES: FC(F)(F)c1c2ccccc2c(-c2cccc3[se]c4c(-c5c6ccccc6c(-c6ccccc6)c6ccccc56)cccc4c23)c2ccccc12 ; N#Cc1c2ccccc2c(-c2cccc3[se]c4c(-c5c6ccccc6c(-c6ccccc6)c6ccccc56)cccc4c23)c2ccccc12]

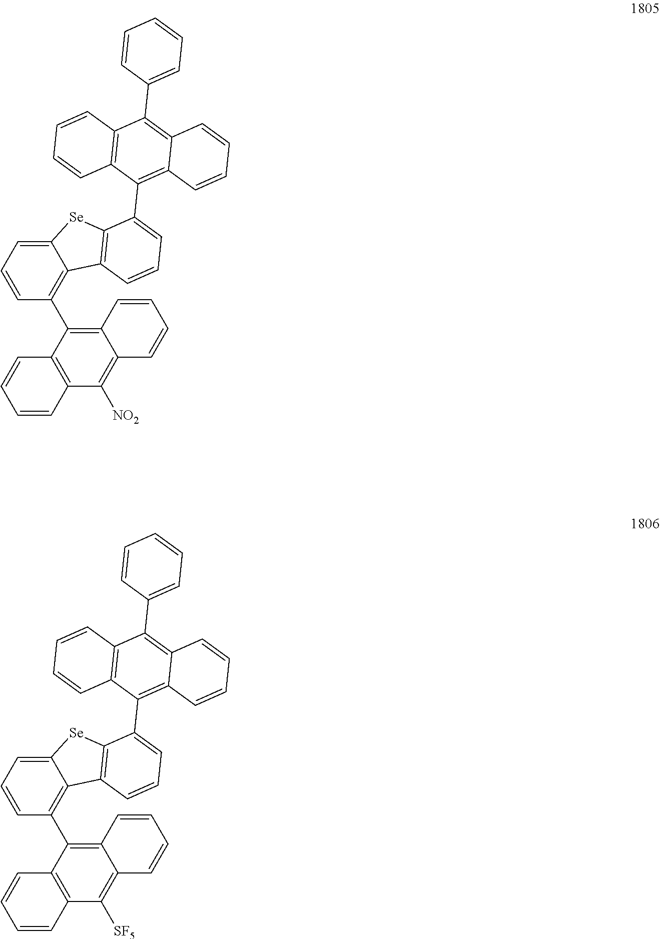

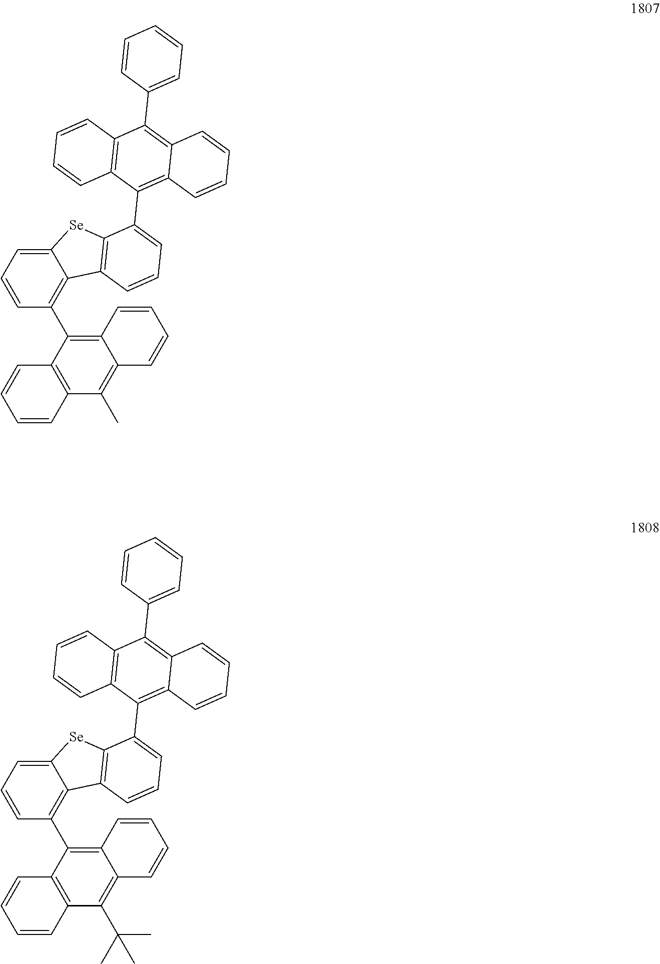 [SMILES: Cc1c2ccccc2c(-c2cccc3[se]c4c(-c5c6ccccc6c(-c6ccccc6)c6ccccc56)cccc4c23)c2ccccc12 ; CC(C)(C)c1c2ccccc2c(-c2cccc3[se]c4c(-c5c6ccccc6c(-c6ccccc6)c6ccccc56)cccc4c23)c2ccccc12]

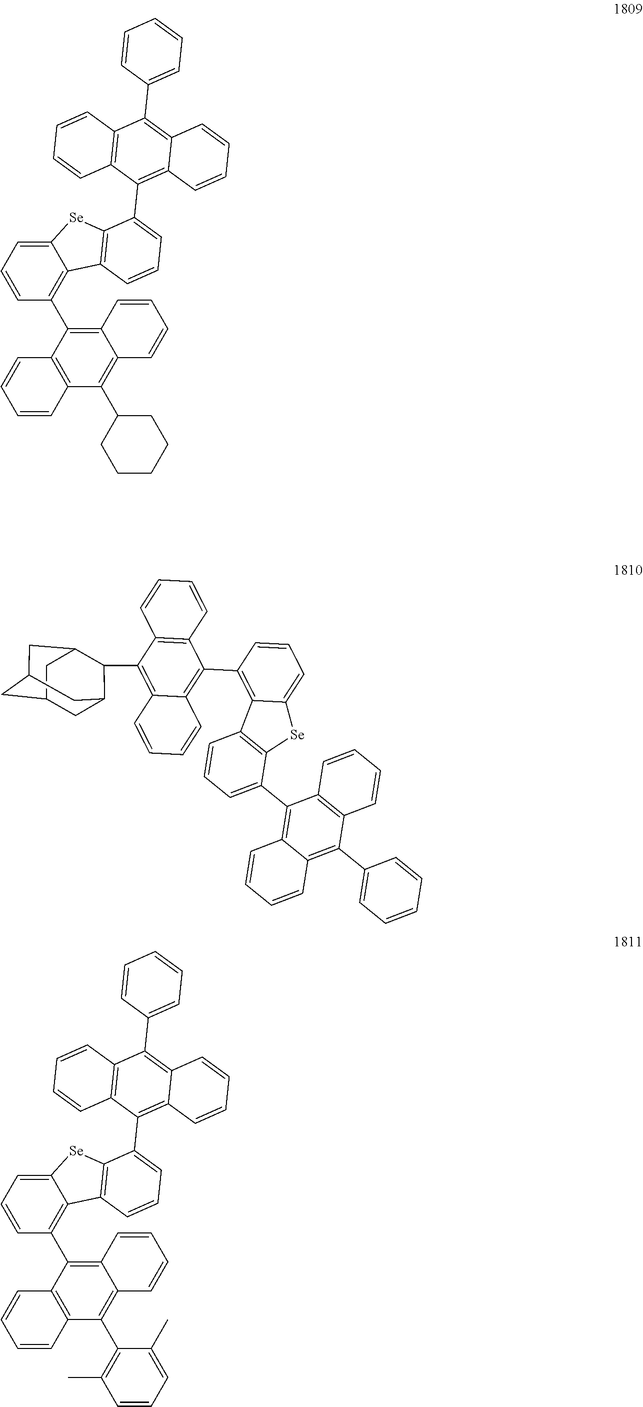 [SMILES: c1ccc(-c2c3ccccc3c(-c3cccc4c3[se]c3cccc(-c5c6ccccc6c(C6CCCCC6)c6ccccc56)c34)c3ccccc23)cc1 ; c1ccc(-c2c3ccccc3c(-c3cccc4c3[se]c3cccc(-c5c6ccccc6c(C6C7CC8CC(C7)CC6C8)c6ccccc56)c34)c3ccccc23)cc1 ; Cc1cccc(C)c1-c1c2ccccc2c(-c2cccc3[se]c4c(-c5c6ccccc6c(-c6ccccc6)c6ccccc56)cccc4c23)c2ccccc12]

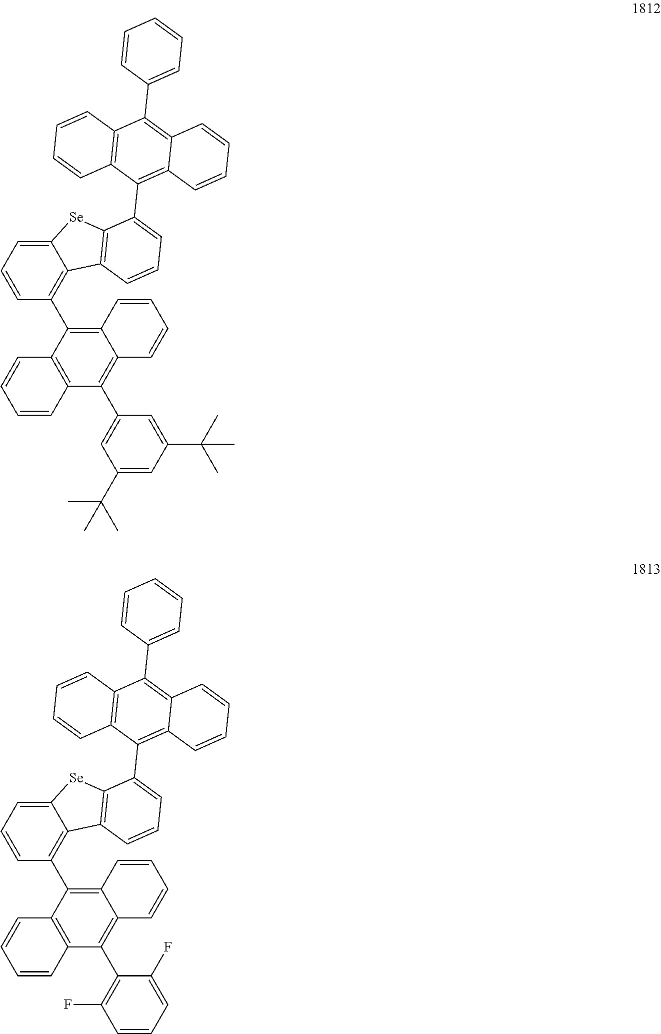 [SMILES: CC(C)(C)c1cc(-c2c3ccccc3c(-c3cccc4[se]c5c(-c6c7ccccc7c(-c7ccccc7)c7ccccc67)cccc5c34)c3ccccc23)cc(C(C)(C)C)c1 ; Fc1cccc(F)c1-c1c2ccccc2c(-c2cccc3[se]c4c(-c5c6ccccc6c(-c6ccccc6)c6ccccc56)cccc4c23)c2ccccc12]

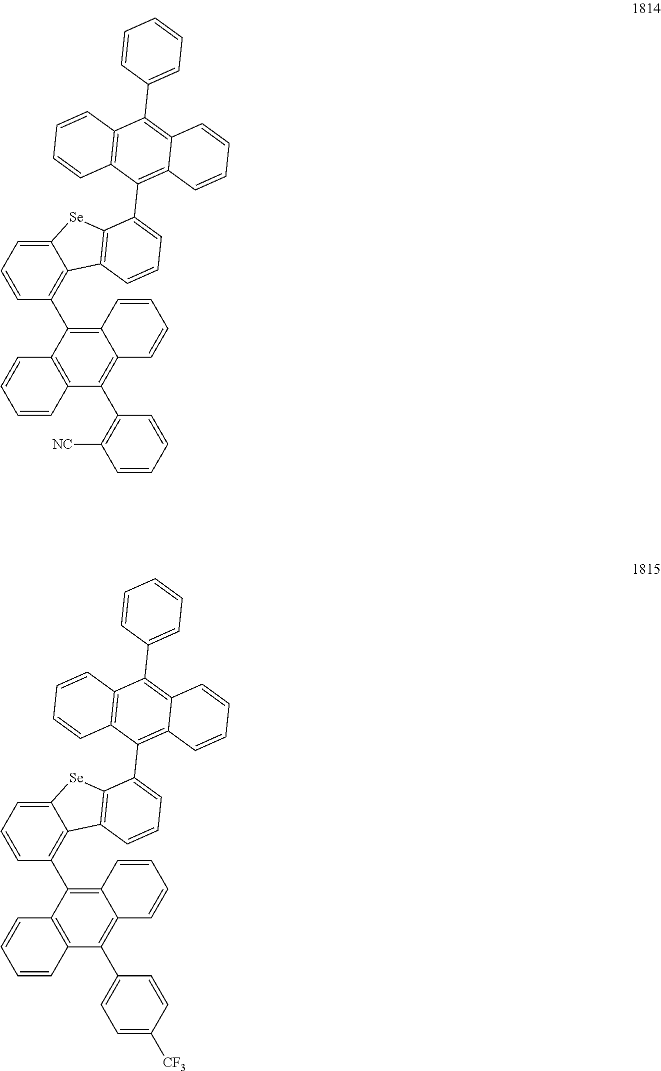 [SMILES: N#Cc1ccccc1-c1c2ccccc2c(-c2cccc3[se]c4c(-c5c6ccccc6c(-c6ccccc6)c6ccccc56)cccc4c23)c2ccccc12 ; FC(F)(F)c1ccc(-c2c3ccccc3c(-c3cccc4[se]c5c(-c6c7ccccc7c(-c7ccccc7)c7ccccc67)cccc5c34)c3ccccc23)cc1]

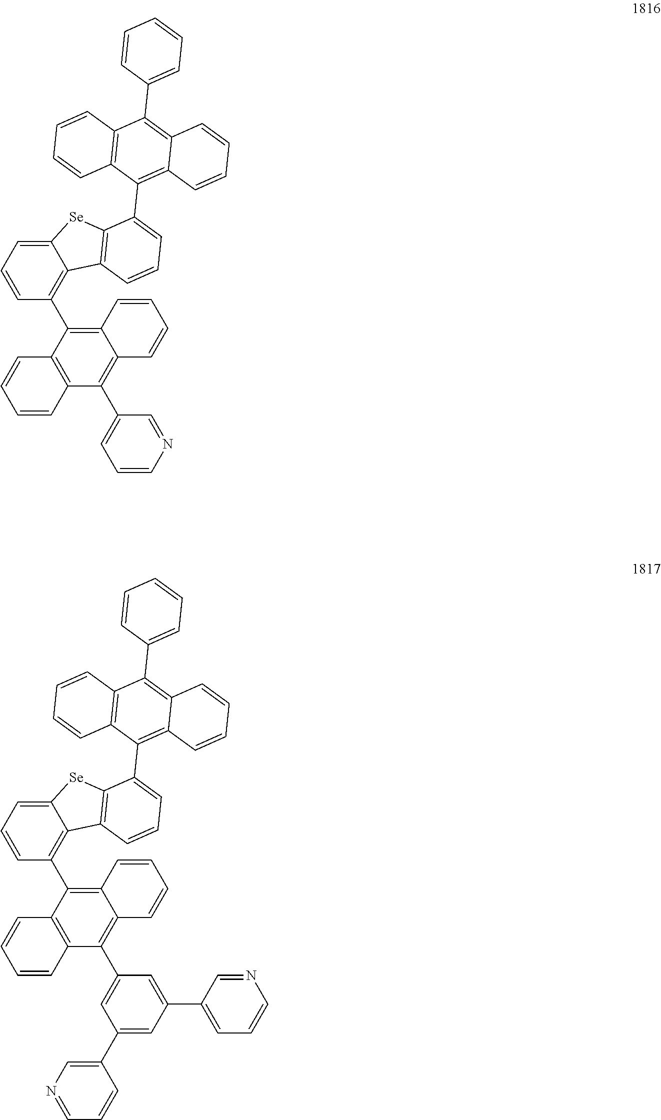 [SMILES: c1ccc(-c2c3ccccc3c(-c3cccc4c3[se]c3cccc(-c5c6ccccc6c(-c6cccnc6)c6ccccc56)c34)c3ccccc23)cc1 ; c1ccc(-c2c3ccccc3c(-c3cccc4c3[se]c3cccc(-c5c6ccccc6c(-c6cc(-c7cccnc7)cc(-c7cccnc7)c6)c6ccccc56)c34)c3ccccc23)cc1]

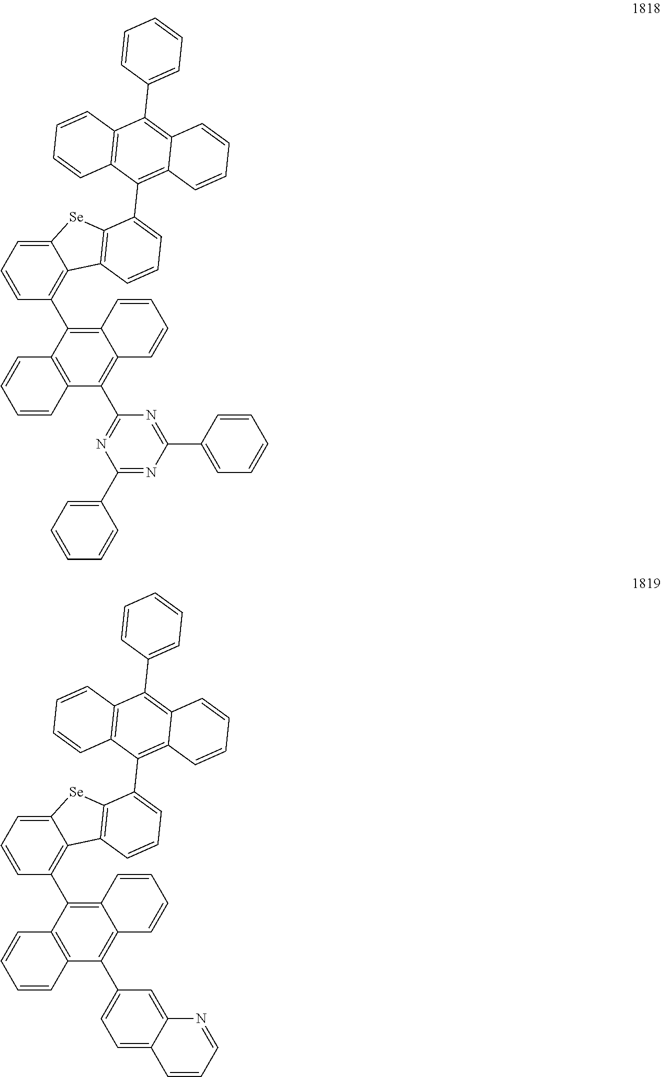 [SMILES: c1ccc(-c2nc(-c3ccccc3)nc(-c3c4ccccc4c(-c4cccc5[se]c6c(-c7c8ccccc8c(-c8ccccc8)c8ccccc78)cccc6c45)c4ccccc34)n2)cc1 ; c1ccc(-c2c3ccccc3c(-c3cccc4c3[se]c3cccc(-c5c6ccccc6c(-c6ccc7cccnc7c6)c6ccccc56)c34)c3ccccc23)cc1]

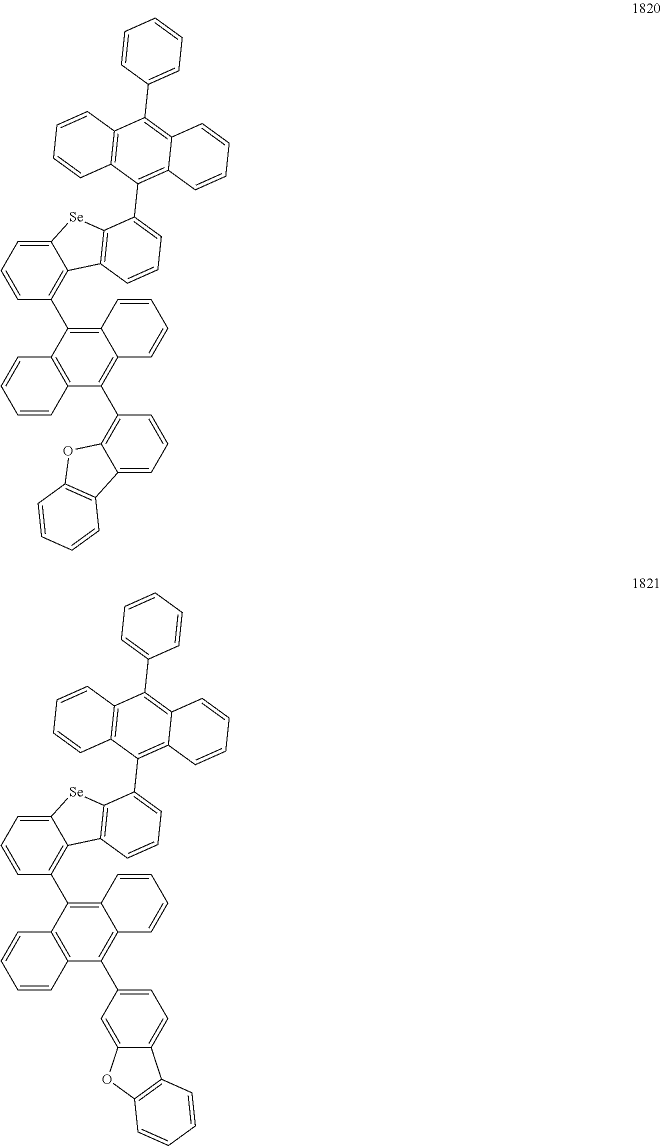 [SMILES: c1ccc(-c2c3ccccc3c(-c3cccc4c3[se]c3cccc(-c5c6ccccc6c(-c6cccc7c6oc6ccccc67)c6ccccc56)c34)c3ccccc23)cc1 ; c1ccc(-c2c3ccccc3c(-c3cccc4c3[se]c3cccc(-c5c6ccccc6c(-c6ccc7c(c6)oc6ccccc67)c6ccccc56)c34)c3ccccc23)cc1]

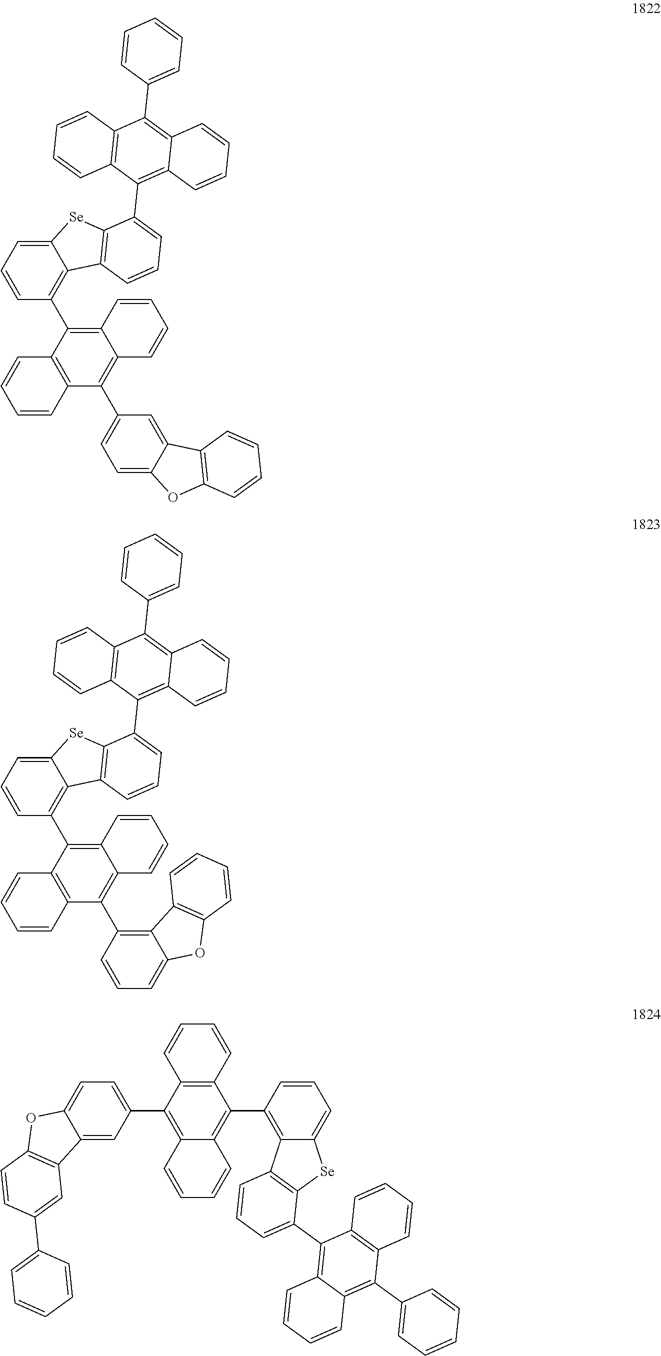

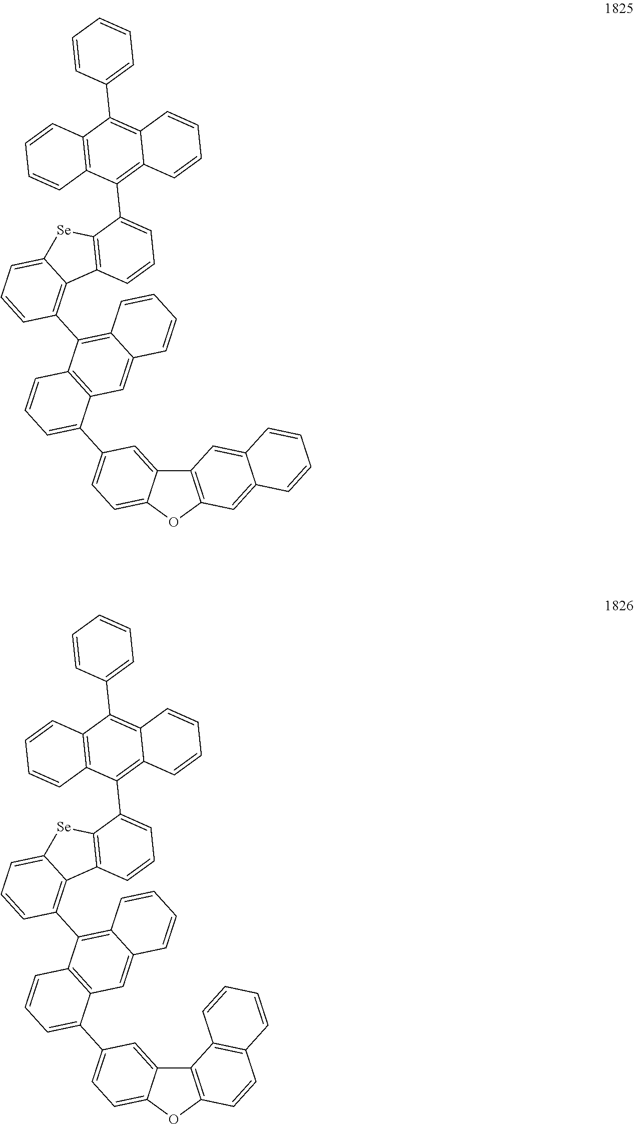 [SMILES: c1ccc(-c2c3ccccc3c(-c3cccc4c3[se]c3cccc(-c5c6ccccc6cc6c(-c7ccc8oc9cc%10ccccc%10cc9c8c7)cccc56)c34)c3ccccc23)cc1 ; c1ccc(-c2c3ccccc3c(-c3cccc4c3[se]c3cccc(-c5c6ccccc6cc6c(-c7ccc8oc9ccc%10ccccc%10c9c8c7)cccc56)c34)c3ccccc23)cc1]

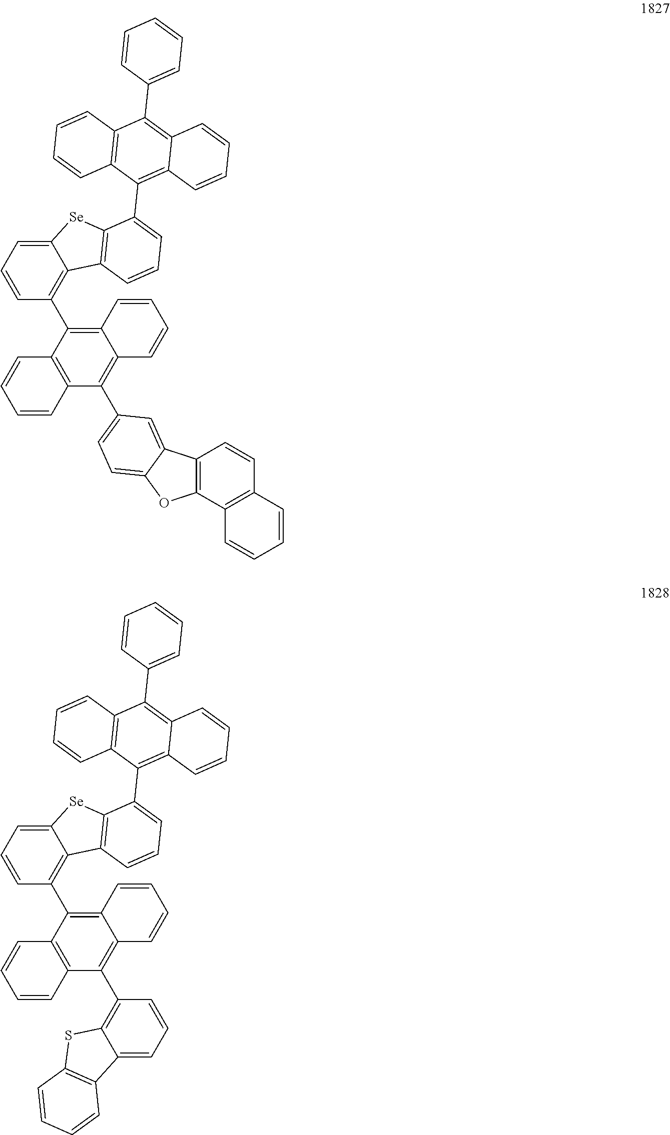 [SMILES: c1ccc(-c2c3ccccc3c(-c3cccc4c3[se]c3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3[se]c3cccc(-c5c6ccccc6c(-c6cccc7c6sc6ccccc67)c6ccccc56)c34)c3ccccc23)cc1]

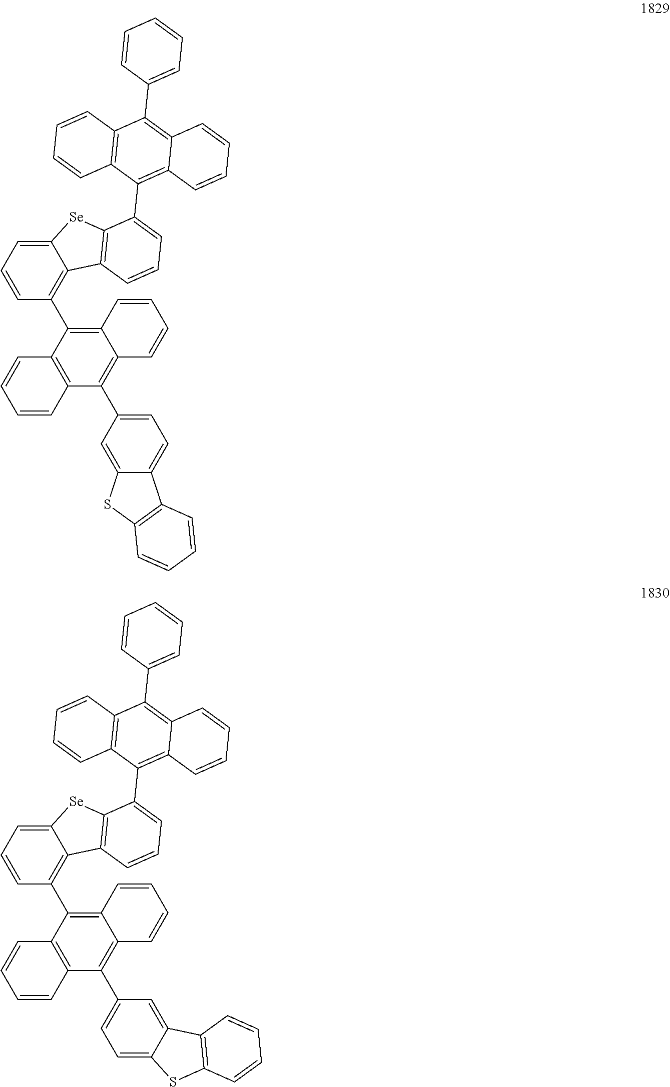

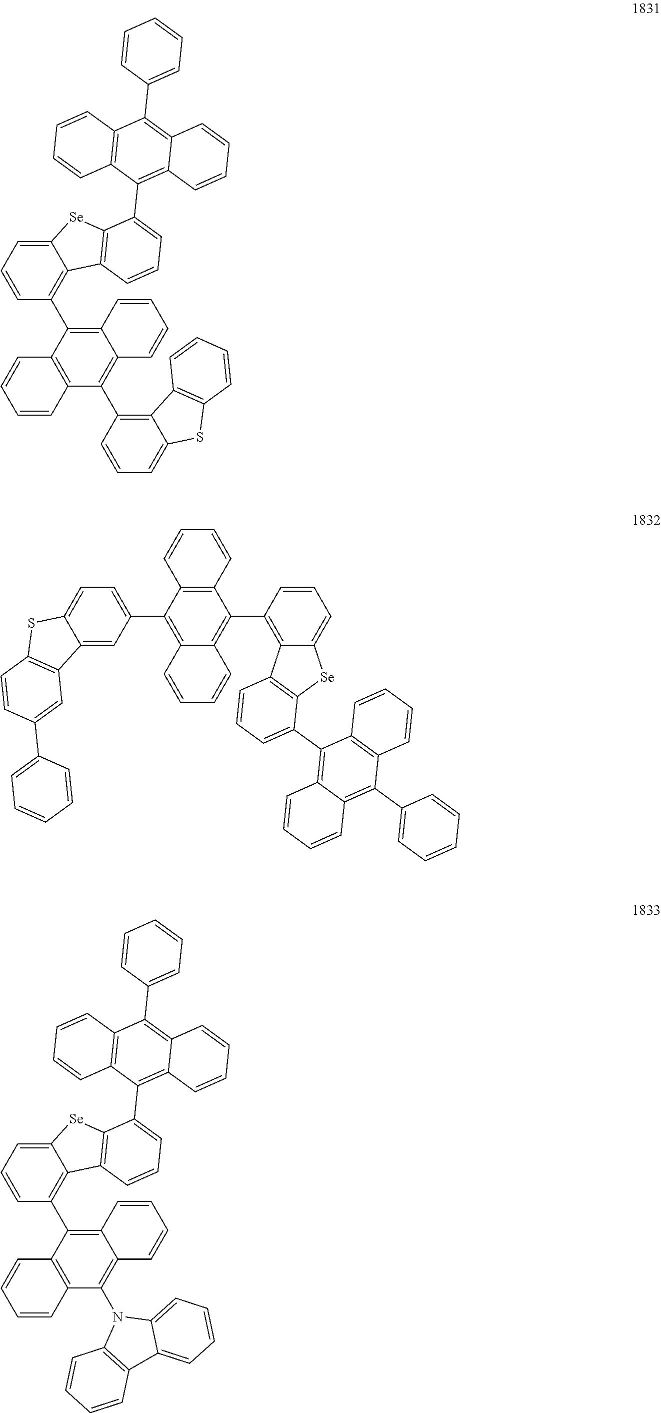 [SMILES: c1ccc(-c2c3ccccc3c(-c3cccc4c3[se]c3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1 ; c1ccc(-c2c3ccccc3c(-c3cccc4c3[se]c3cccc(-c5c6ccccc6c(-n6c7ccccc7c7ccccc76)c6ccccc56)c34)c3ccccc23)cc1]

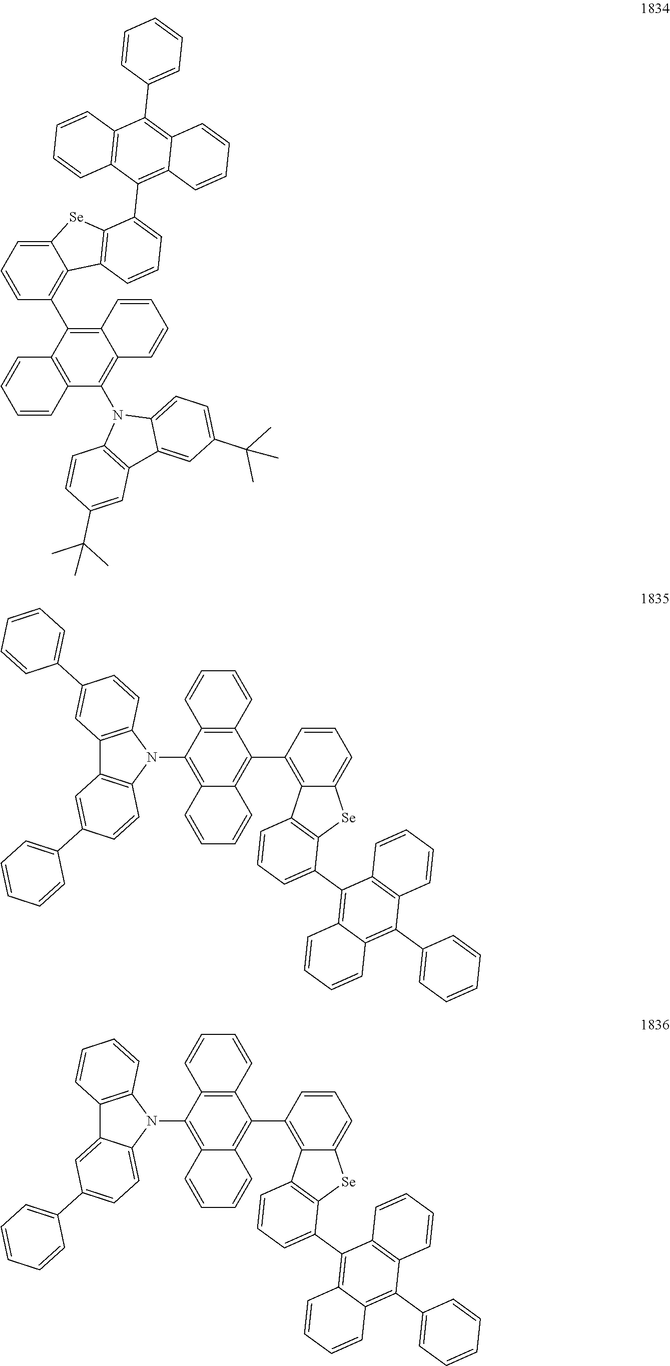 [SMILES: CC(C)(C)c1ccc2c(c1)c1cc(C(C)(C)C)ccc1n2-c1c2ccccc2c(-c2cccc3[se]c4c(-c5c6ccccc6c(-c6ccccc6)c6ccccc56)cccc4c23)c2ccccc12 ; c1ccc(-c2ccc3c(c2)c2cc(-c4ccccc4)ccc2n3-c2c3ccccc3c(-c3cccc4[se]c5c(-c6c7ccccc7c(-c7ccccc7)c7ccccc67)cccc5c34)c3ccccc23)cc1 ; c1ccc(-c2ccc3c(c2)c2ccccc2n3-c2c3ccccc3c(-c3cccc4[se]c5c(-c6c7ccccc7c(-c7ccccc7)c7ccccc67)cccc5c34)c3ccccc23)cc1]

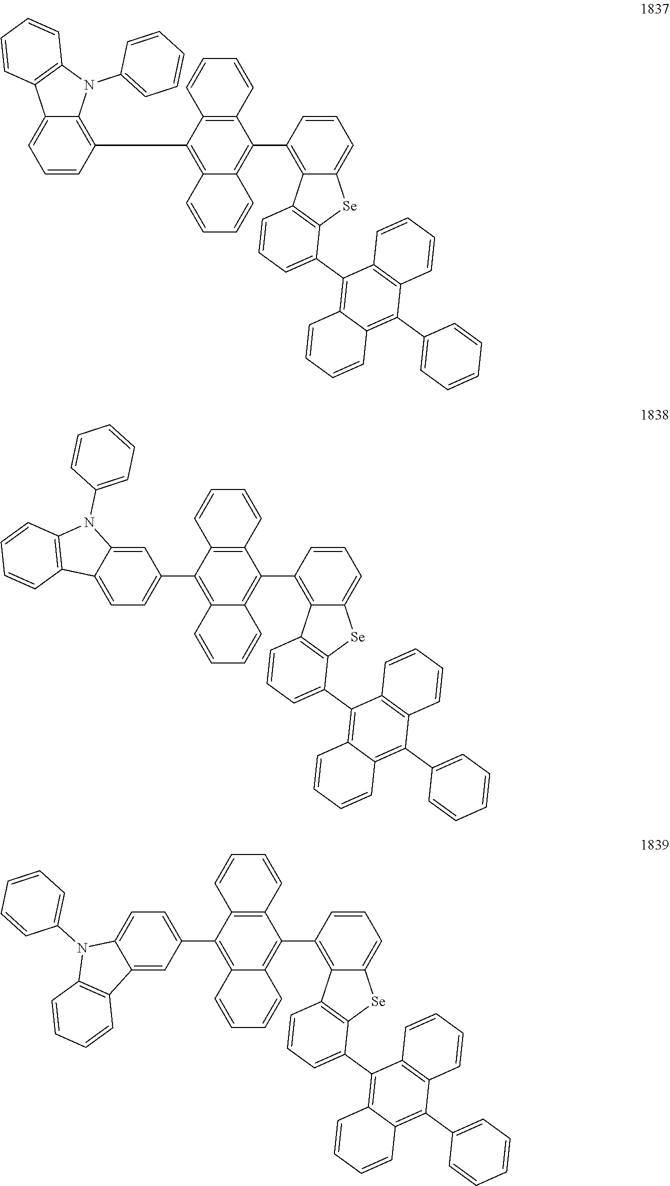 [SMILES: c1ccc(-c2c3ccccc3c(-c3cccc4c3[se]c3cccc(-c5c6ccccc6c(-c6cccc7c8ccccc8n(-c8ccccc8)c67)c6ccccc56)c34)c3ccccc23)cc1 ; c1ccc(-c2c3ccccc3c(-c3cccc4c3[se]c3cccc(-c5c6ccccc6c(-c6ccc7c8ccccc8n(-c8ccccc8)c7c6)c6ccccc56)c34)c3ccccc23)cc1 ; c1ccc(-c2c3ccccc3c(-c3cccc4c3[se]c3cccc(-c5c6ccccc6c(-c6ccc7c(c6)c6ccccc6n7-c6ccccc6)c6ccccc56)c34)c3ccccc23)cc1]

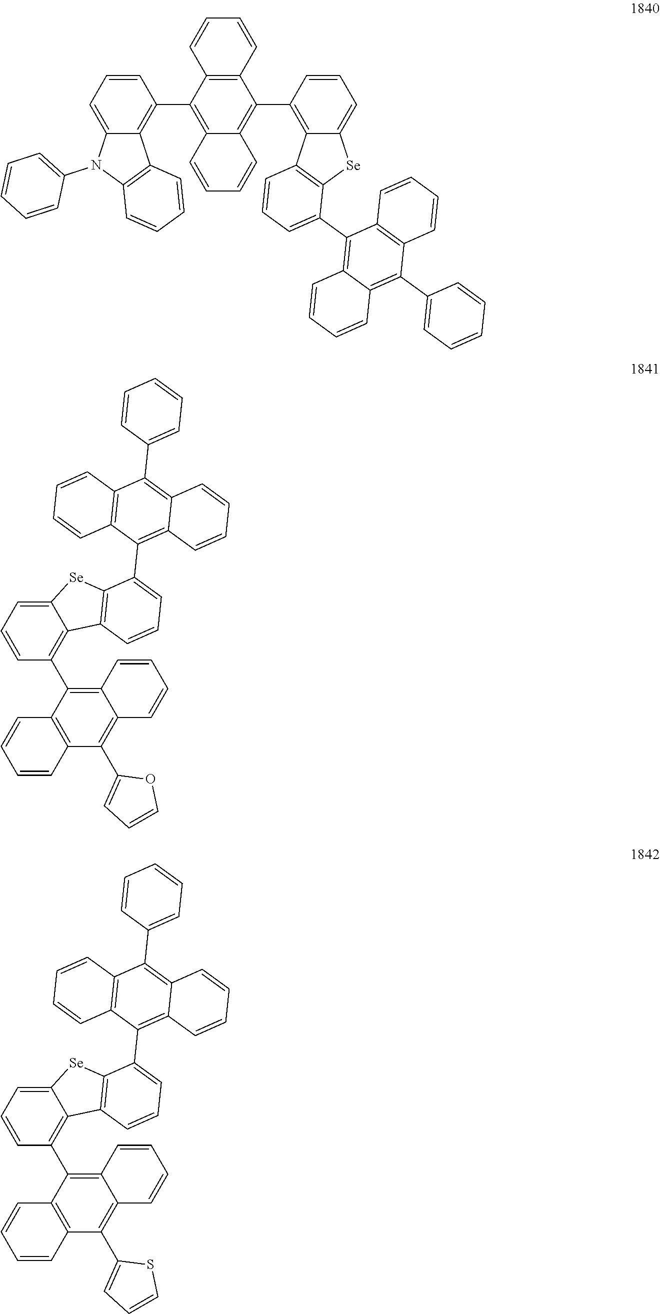

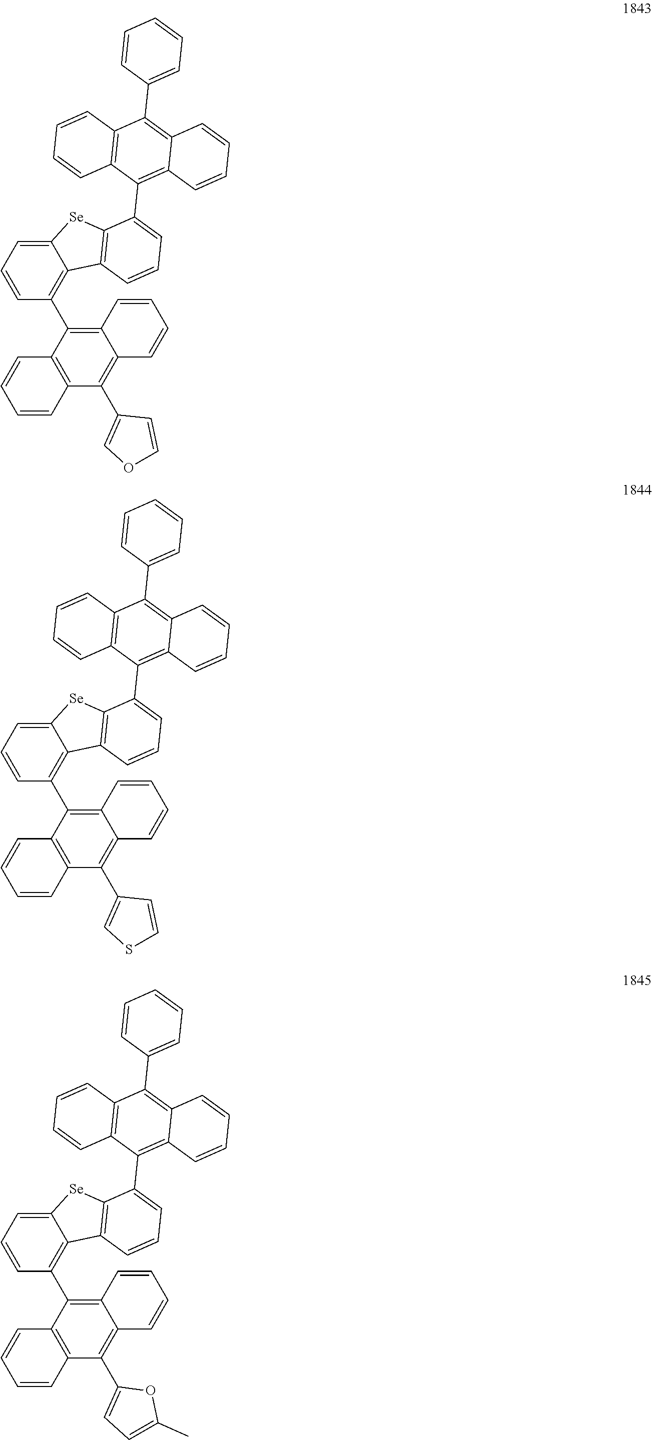

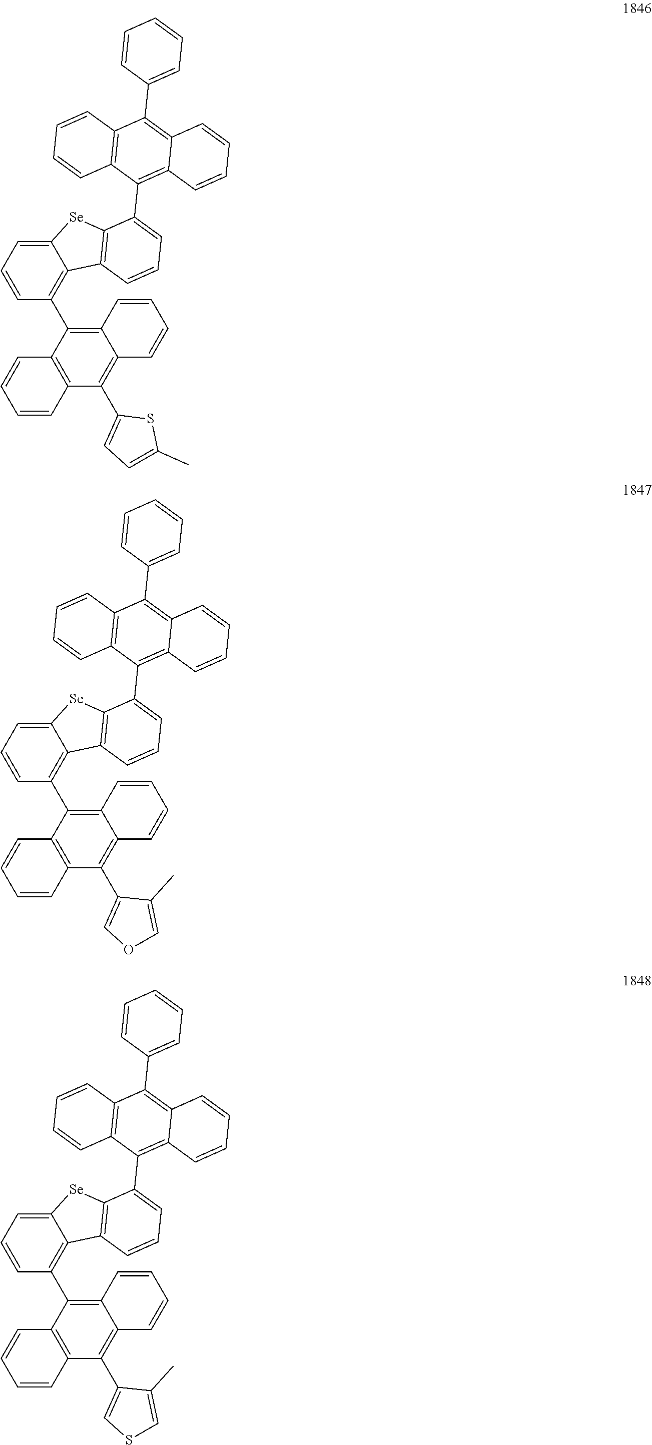

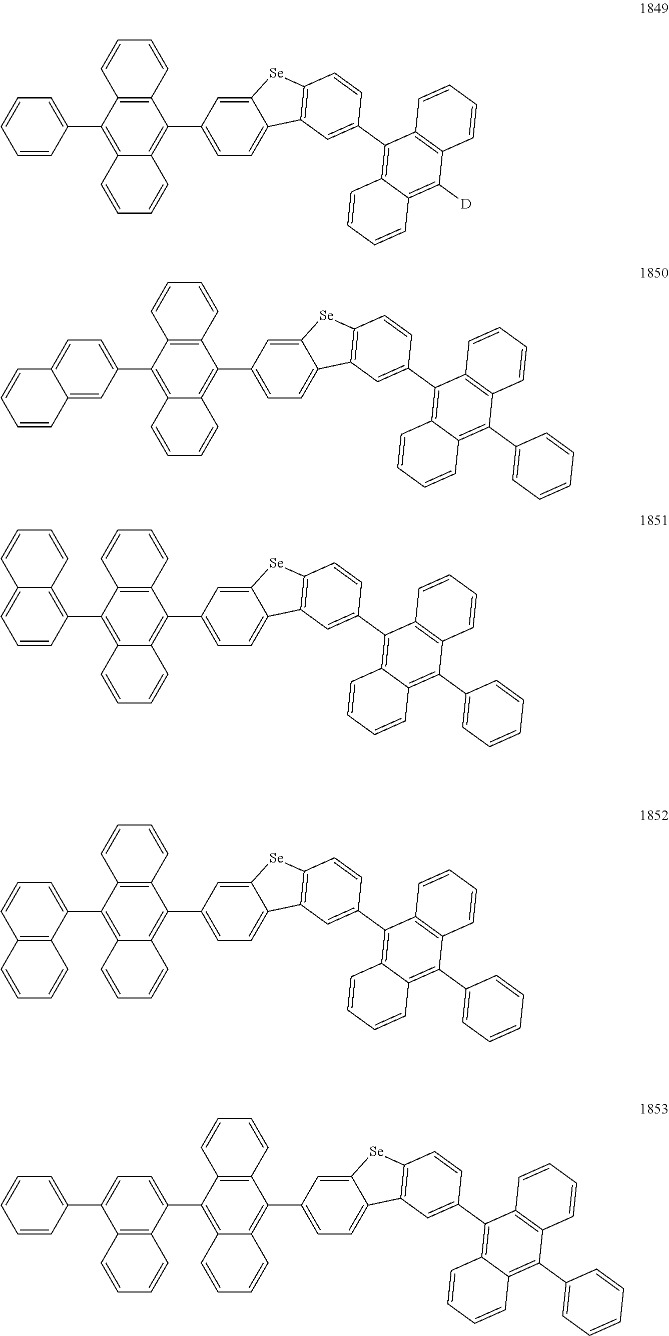 [SMILES: [2H]c1c2ccccc2c(-c2ccc3[se]c4cc(-c5c6ccccc6c(-c6ccccc6)c6ccccc56)ccc4c3c2)c2ccccc12 ; c1ccc(-c2c3ccccc3c(-c3ccc4[se]c5cc(-c6c7ccccc7c(-c7ccc8ccccc8c7)c7ccccc67)ccc5c4c3)c3ccccc23)cc1 ; c1ccc(-c2c3ccccc3c(-c3ccc4[se]c5cc(-c6c7ccccc7c(-c7cccc8ccccc78)c7ccccc67)ccc5c4c3)c3ccccc23)cc1 ; c1ccc(-c2c3ccccc3c(-c3ccc4[se]c5cc(-c6c7ccccc7c(-c7cccc8ccccc78)c7ccccc67)ccc5c4c3)c3ccccc23)cc1 ; c1ccc(-c2ccc(-c3c4ccccc4c(-c4ccc5c(c4)[se]c4ccc(-c6c7ccccc7c(-c7ccccc7)c7ccccc67)cc45)c4ccccc34)c3ccccc23)cc1]

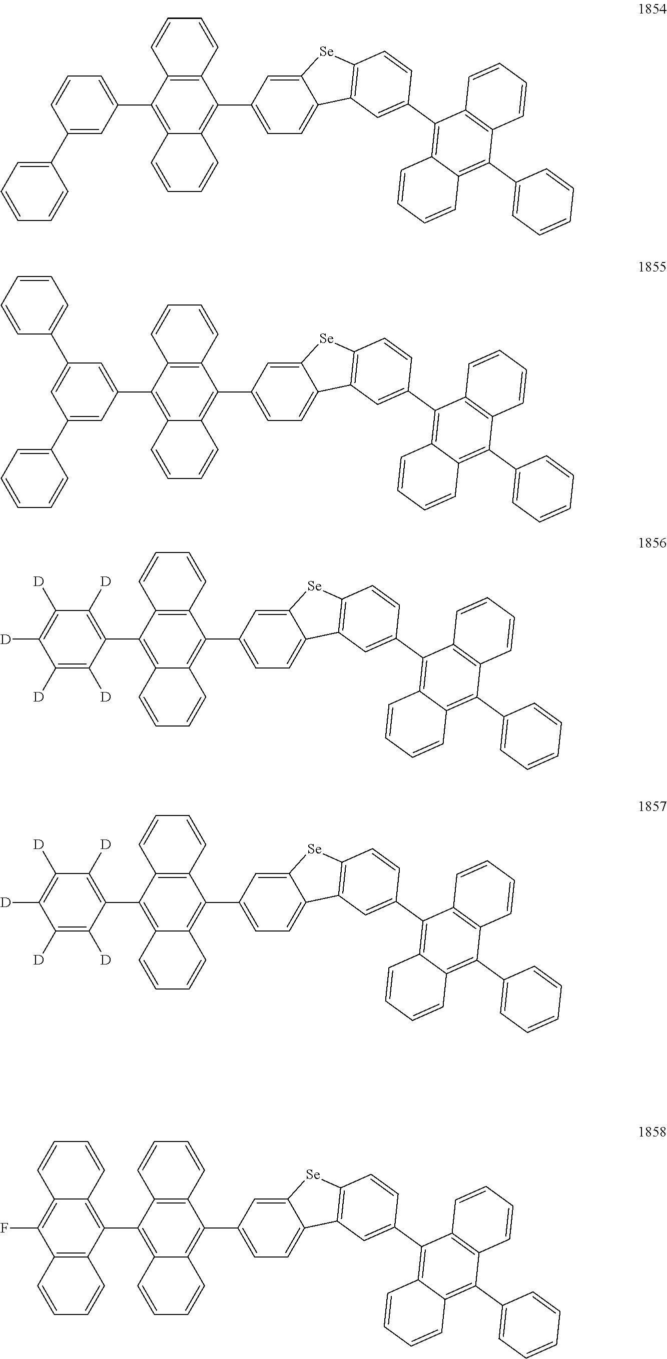

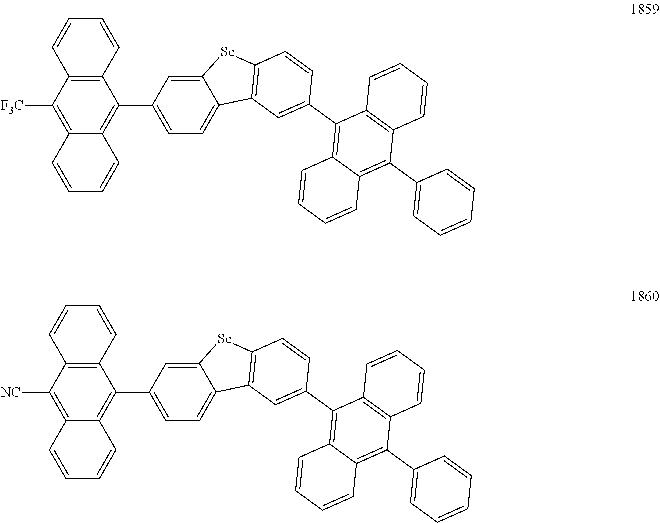 [SMILES: FC(F)(F)c1c2ccccc2c(-c2ccc3c(c2)[se]c2ccc(-c4c5ccccc5c(-c5ccccc5)c5ccccc45)cc23)c2ccccc12 ; N#Cc1c2ccccc2c(-c2ccc3c(c2)[se]c2ccc(-c4c5ccccc5c(-c5ccccc5)c5ccccc45)cc23)c2ccccc12]

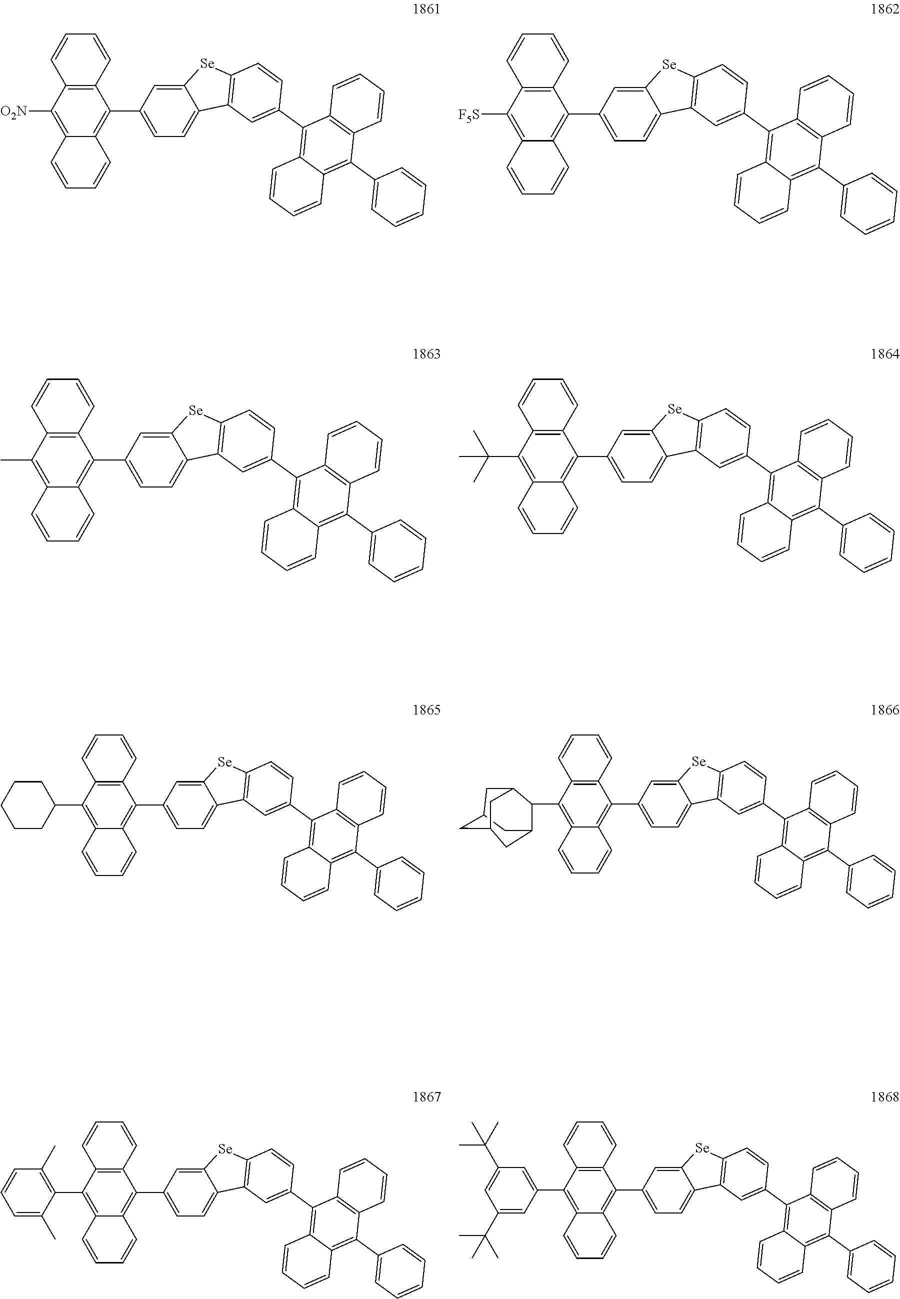

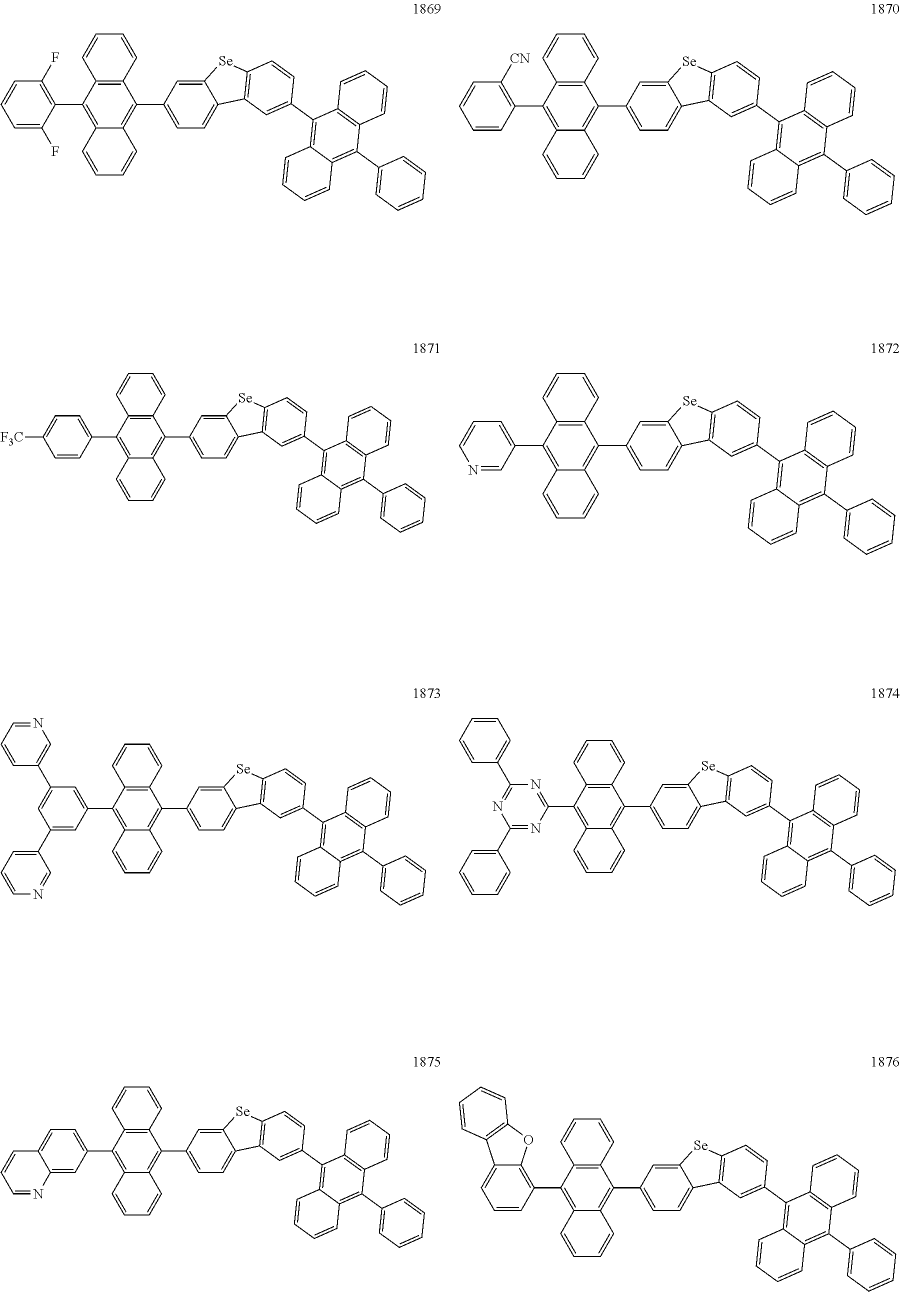

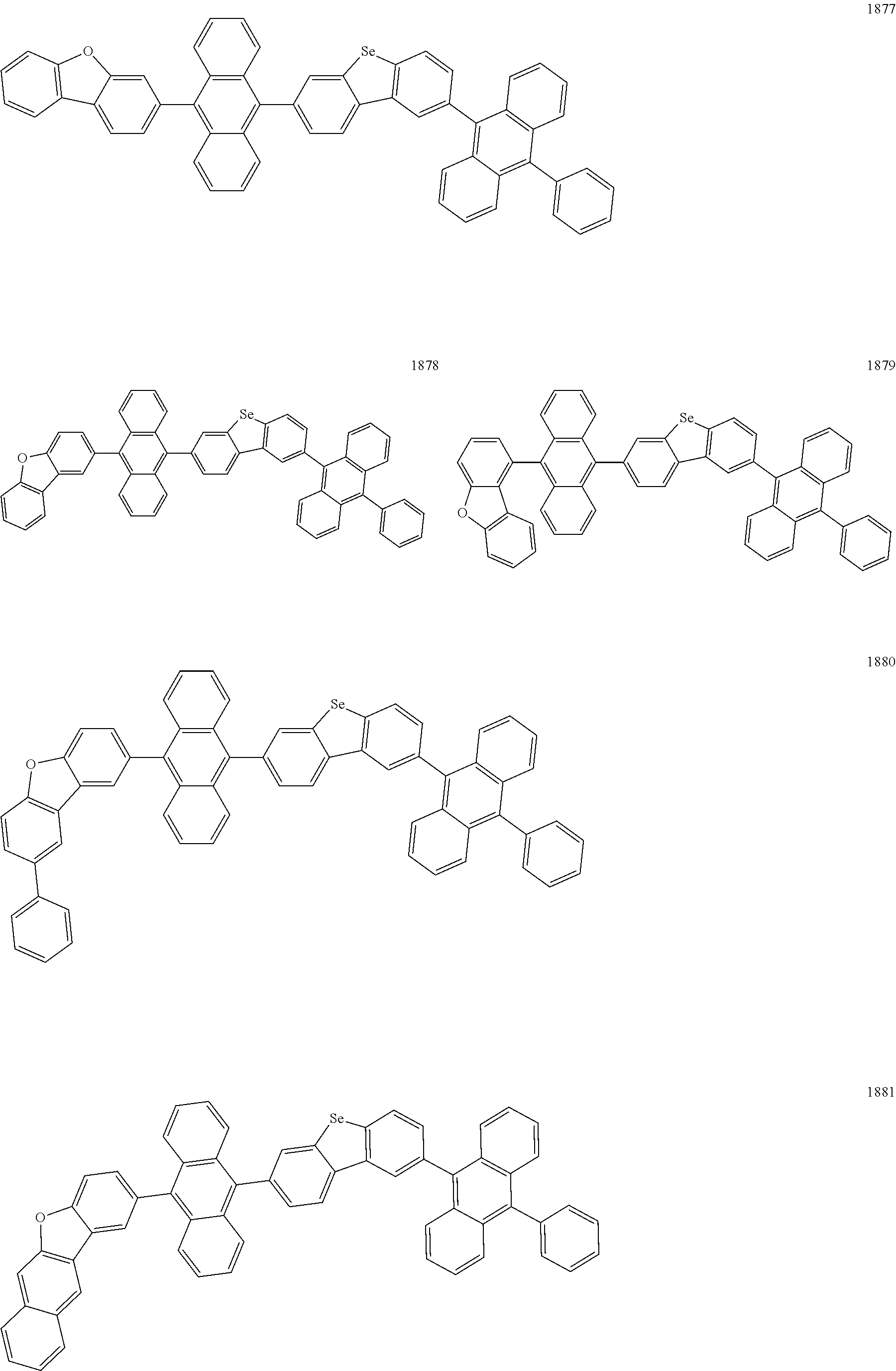

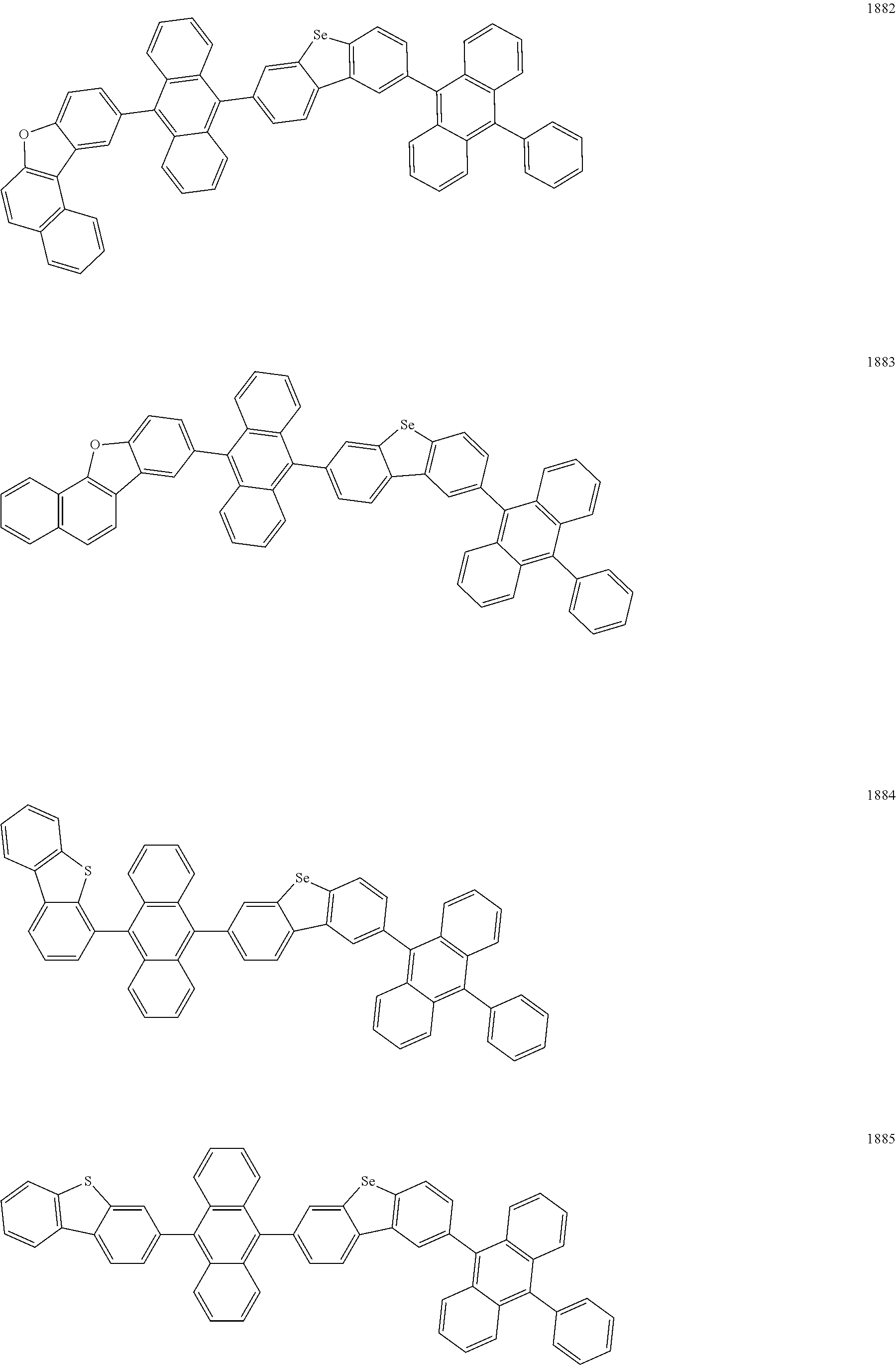 [SMILES: c1ccc(-c2c3ccccc3c(-c3ccc4[se]c5cc(-c6c7ccccc7c(-c7ccc8oc9ccc%10ccccc%10c9c8c7)c7ccccc67)ccc5c4c3)c3ccccc23)cc1 ; c1ccc(-c2c3ccccc3c(-c3ccc4[se]c5cc(-c6c7ccccc7c(-c7ccc8oc9c%10ccccc%10ccc9c8c7)c7ccccc67)ccc5c4c3)c3ccccc23)cc1 ; c1ccc(-c2c3ccccc3c(-c3ccc4[se]c5cc(-c6c7ccccc7c(-c7cccc8c7sc7ccccc78)c7ccccc67)ccc5c4c3)c3ccccc23)cc1 ; c1ccc(-c2c3ccccc3c(-c3ccc4[se]c5cc(-c6c7ccccc7c(-c7ccc8c(c7)sc7ccccc78)c7ccccc67)ccc5c4c3)c3ccccc23)cc1]

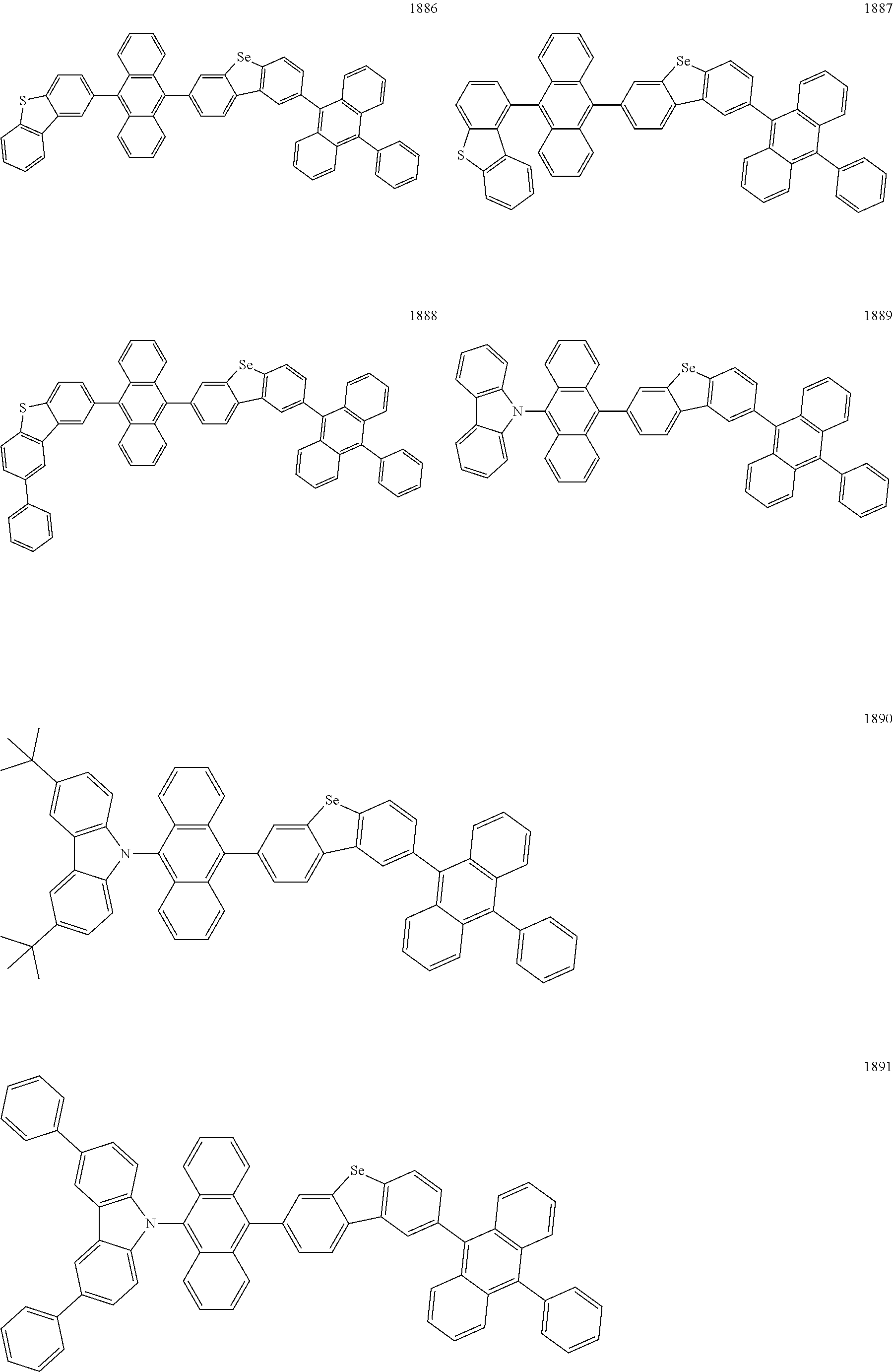

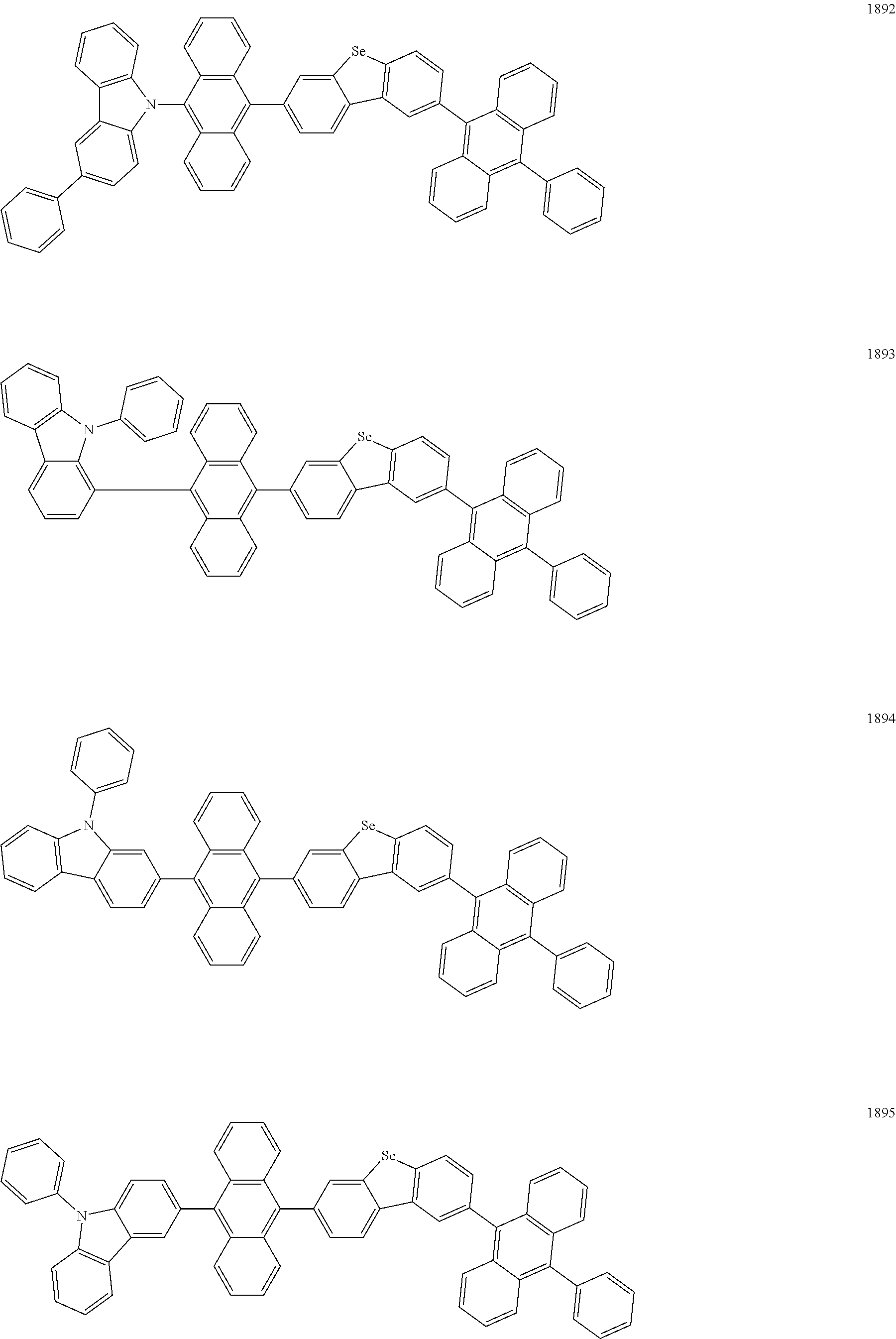 [SMILES: c1ccc(-c2ccc3c(c2)c2ccccc2n3-c2c3ccccc3c(-c3ccc4c(c3)[se]c3ccc(-c5c6ccccc6c(-c6ccccc6)c6ccccc56)cc34)c3ccccc23)cc1 ; c1ccc(-c2c3ccccc3c(-c3ccc4[se]c5cc(-c6c7ccccc7c(-c7cccc8c9ccccc9n(-c9ccccc9)c78)c7ccccc67)ccc5c4c3)c3ccccc23)cc1 ; c1ccc(-c2c3ccccc3c(-c3ccc4[se]c5cc(-c6c7ccccc7c(-c7ccc8c9ccccc9n(-c9ccccc9)c8c7)c7ccccc67)ccc5c4c3)c3ccccc23)cc1 ; c1ccc(-c2c3ccccc3c(-c3ccc4[se]c5cc(-c6c7ccccc7c(-c7ccc8c(c7)c7ccccc7n8-c7ccccc7)c7ccccc67)ccc5c4c3)c3ccccc23)cc1]

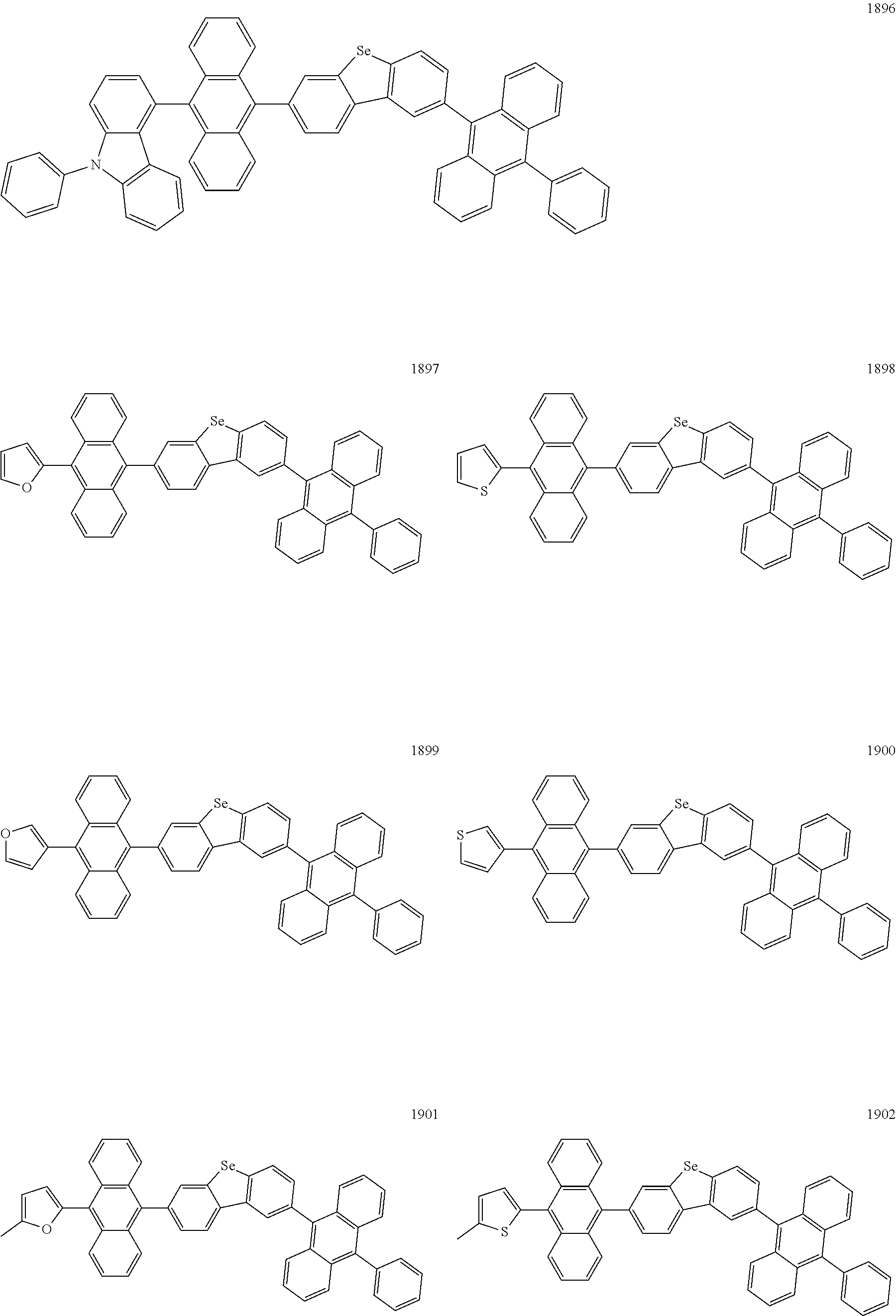

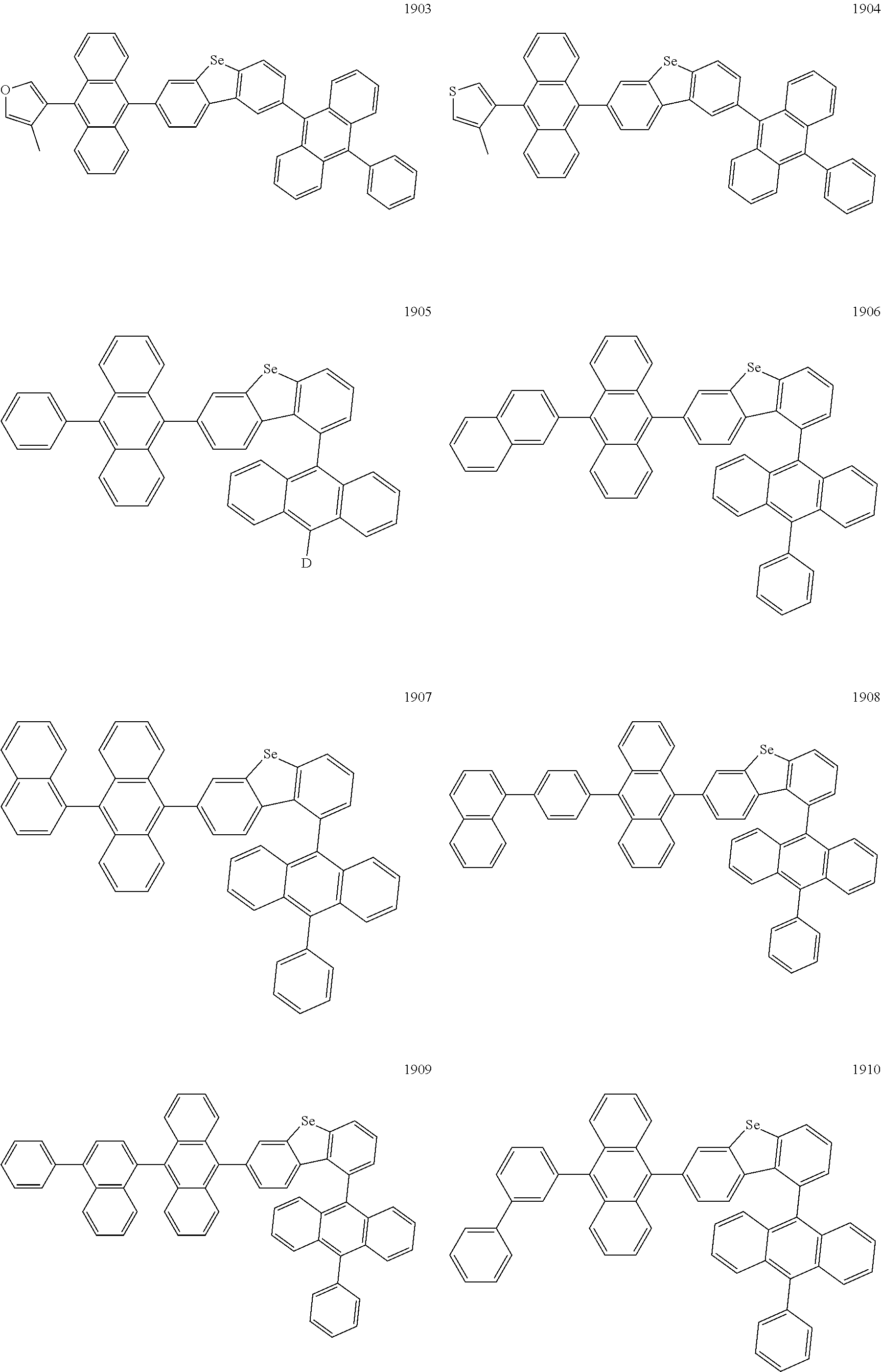

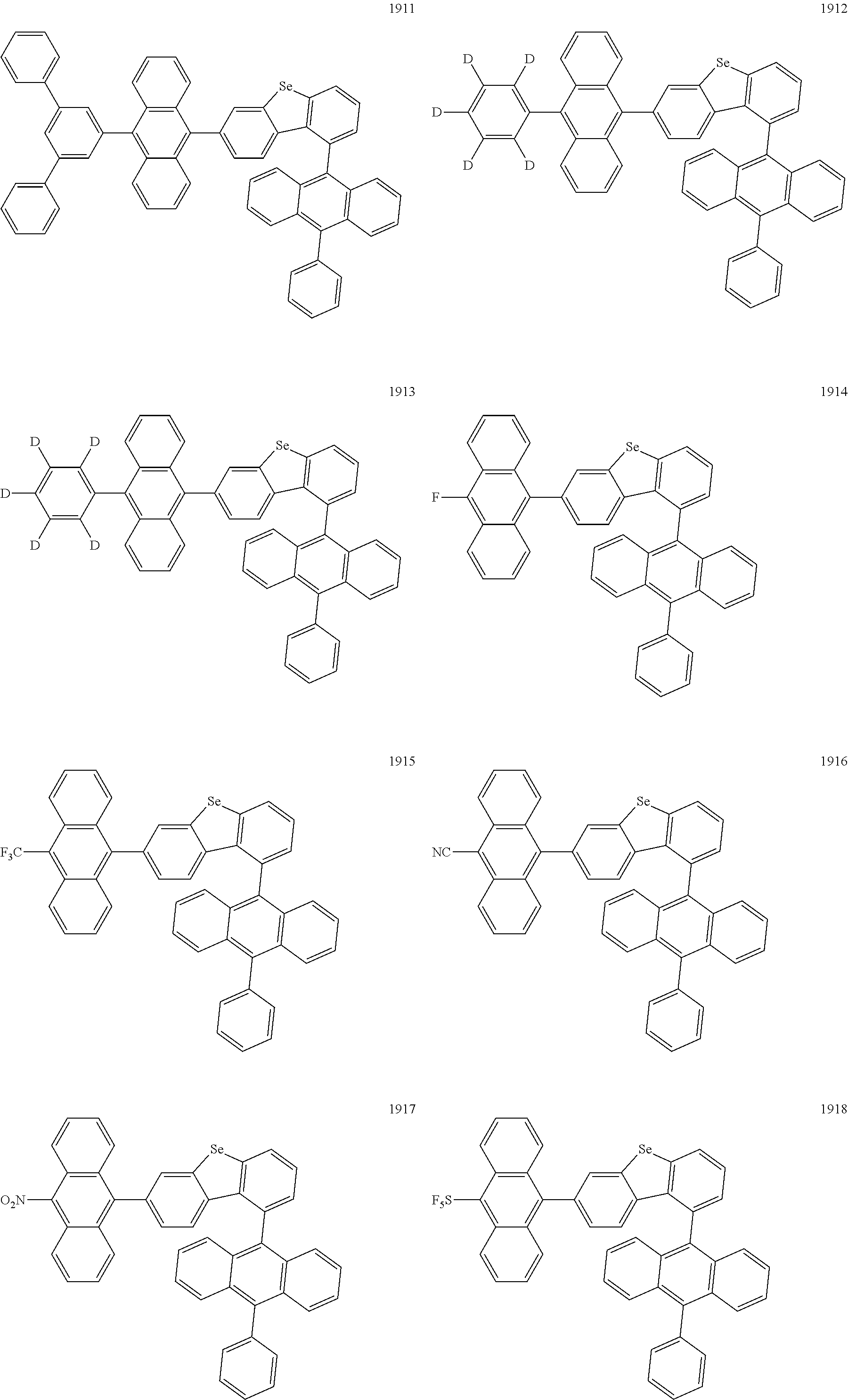

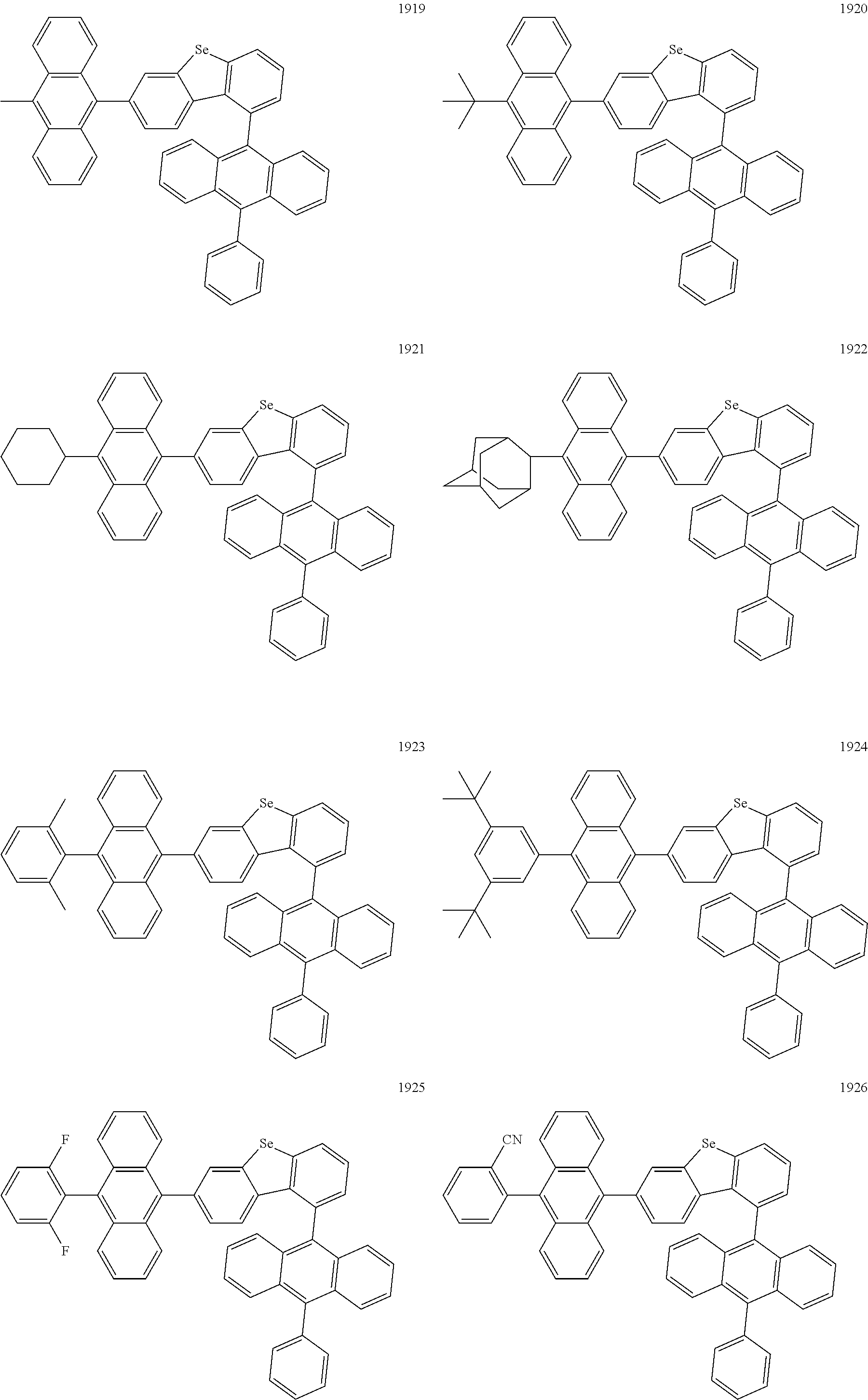

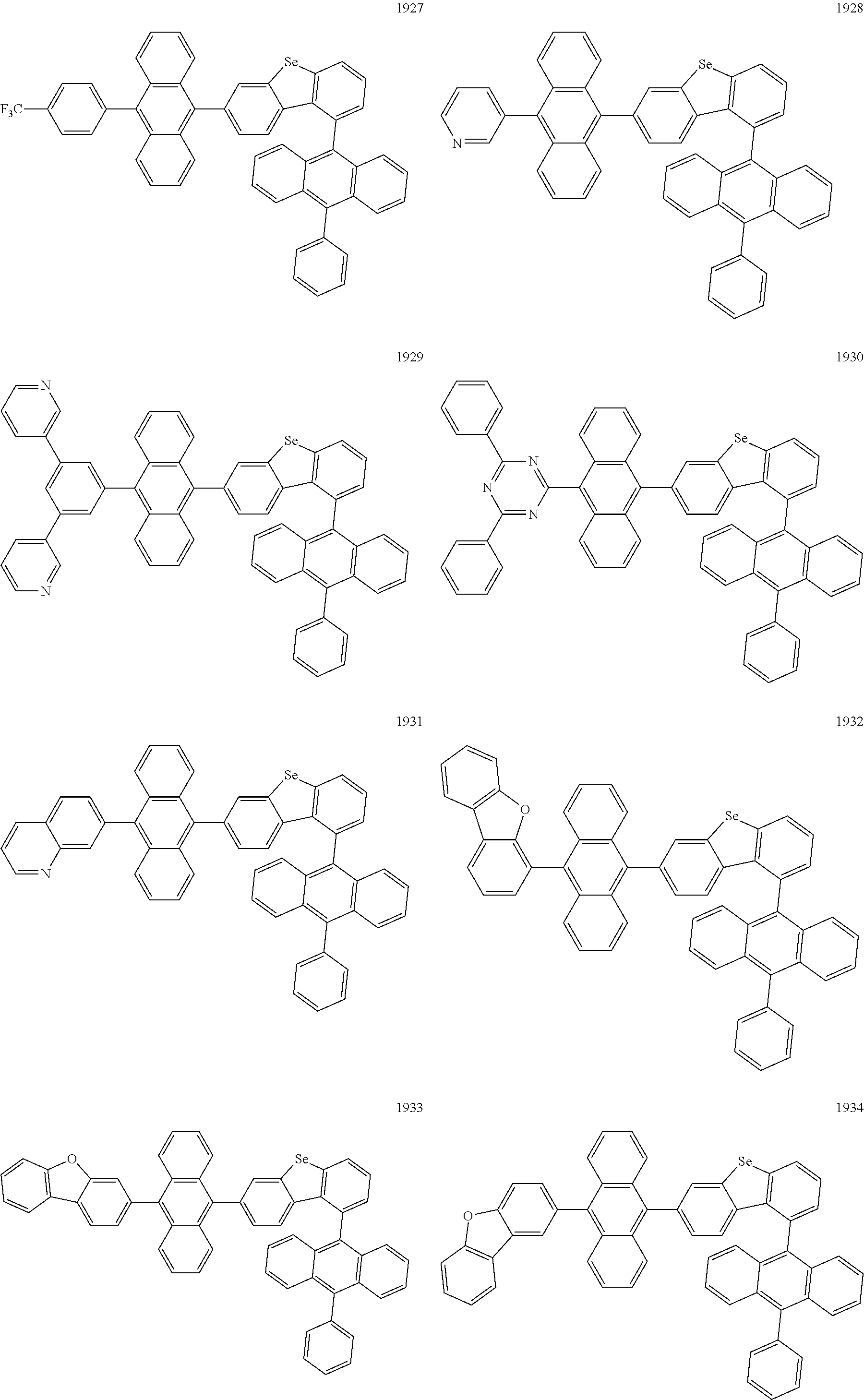

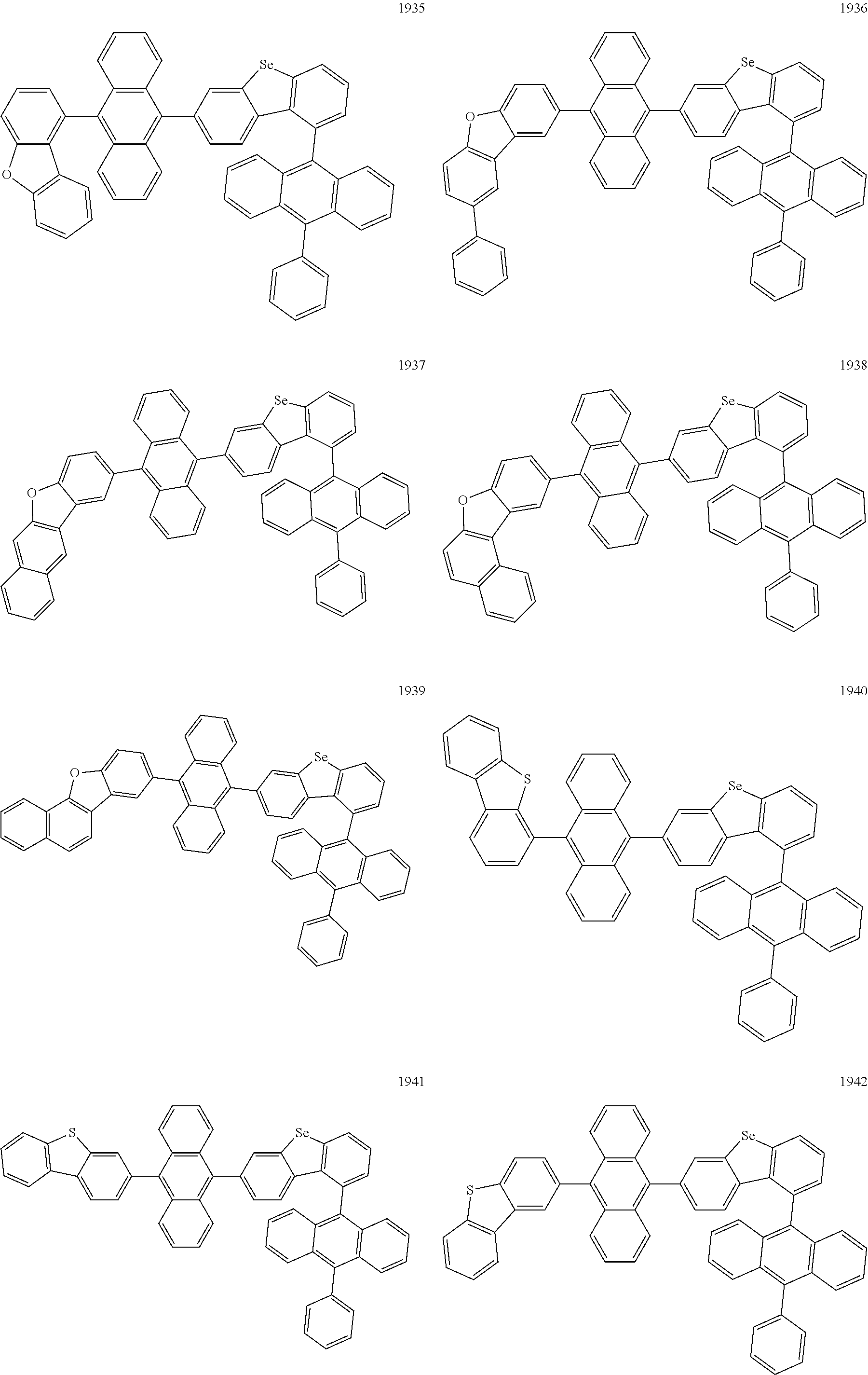

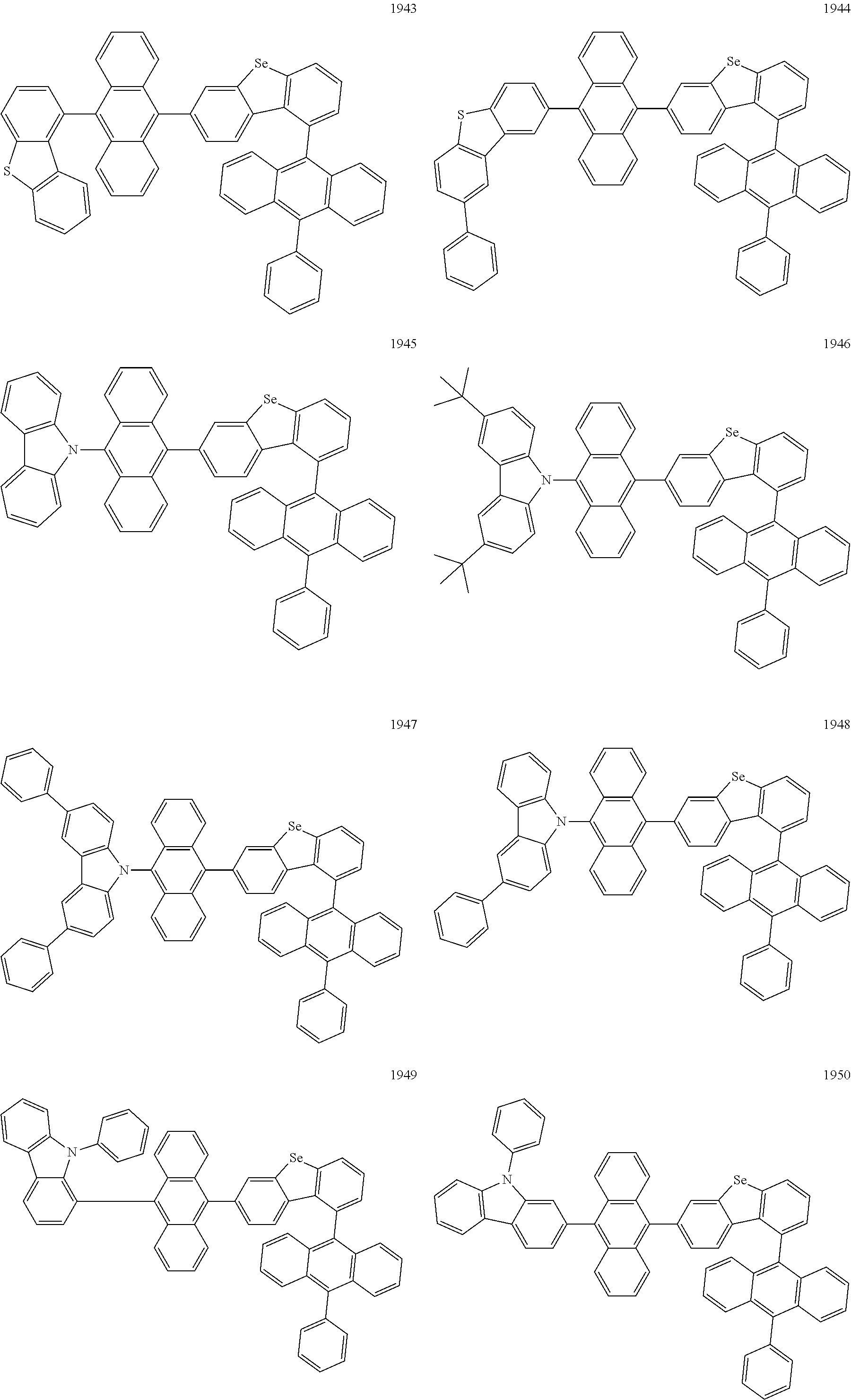

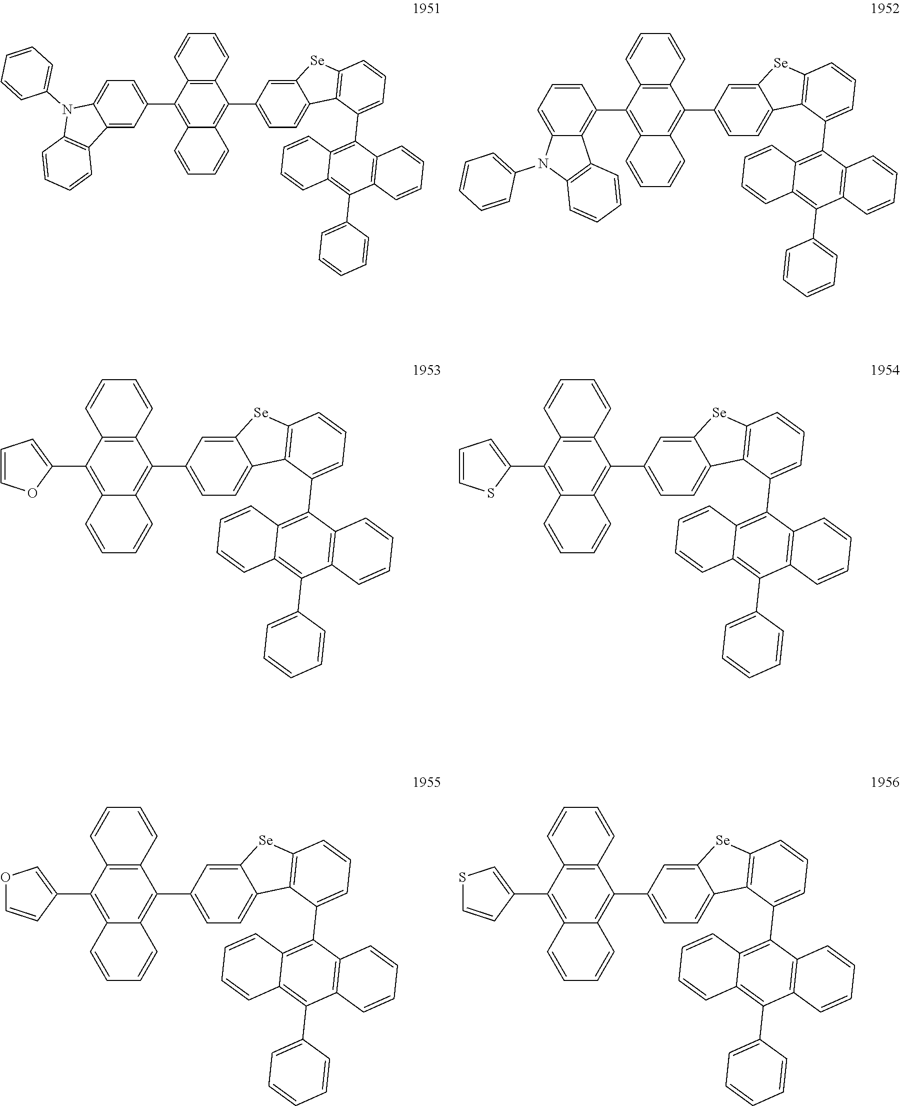

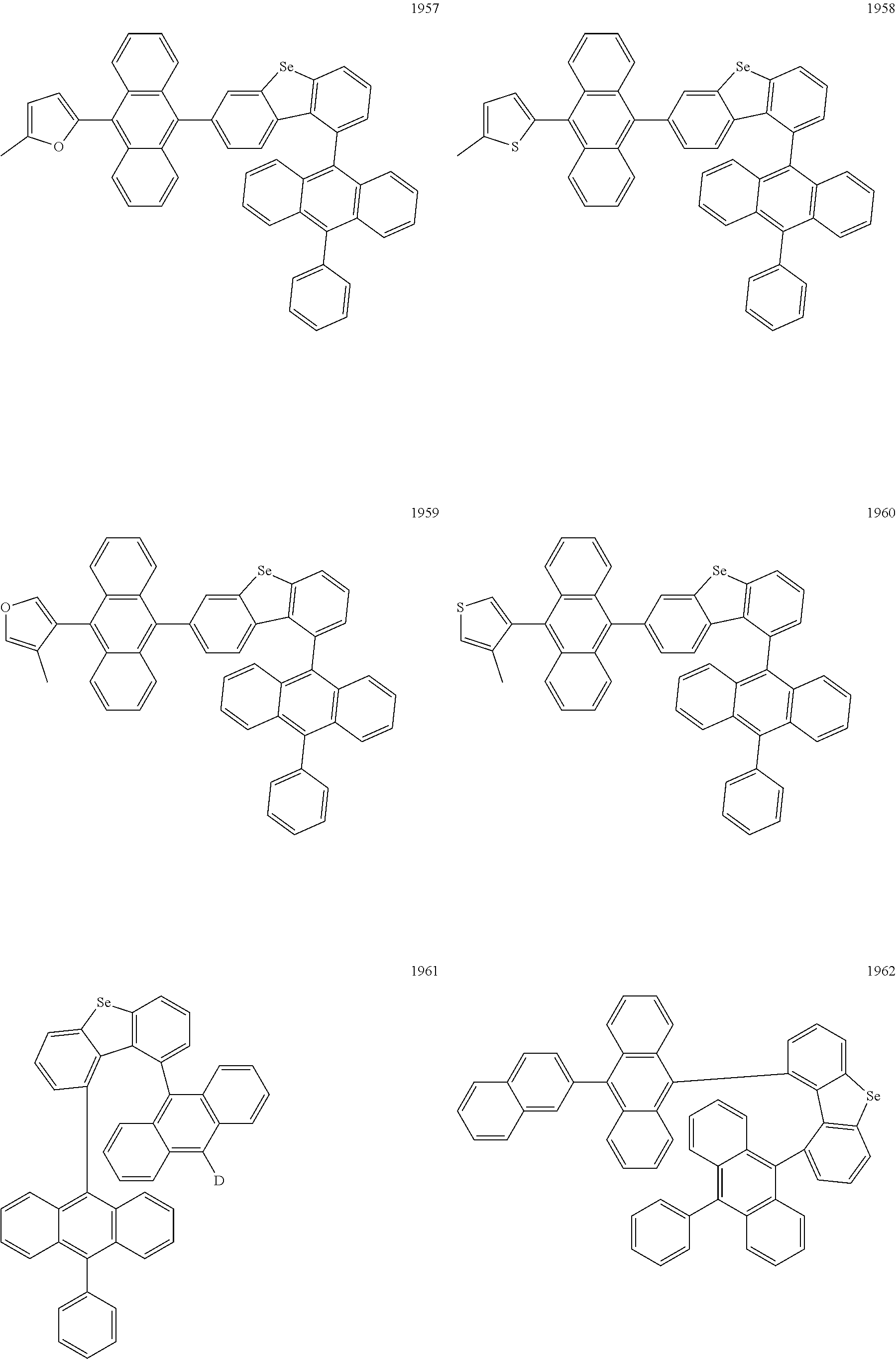

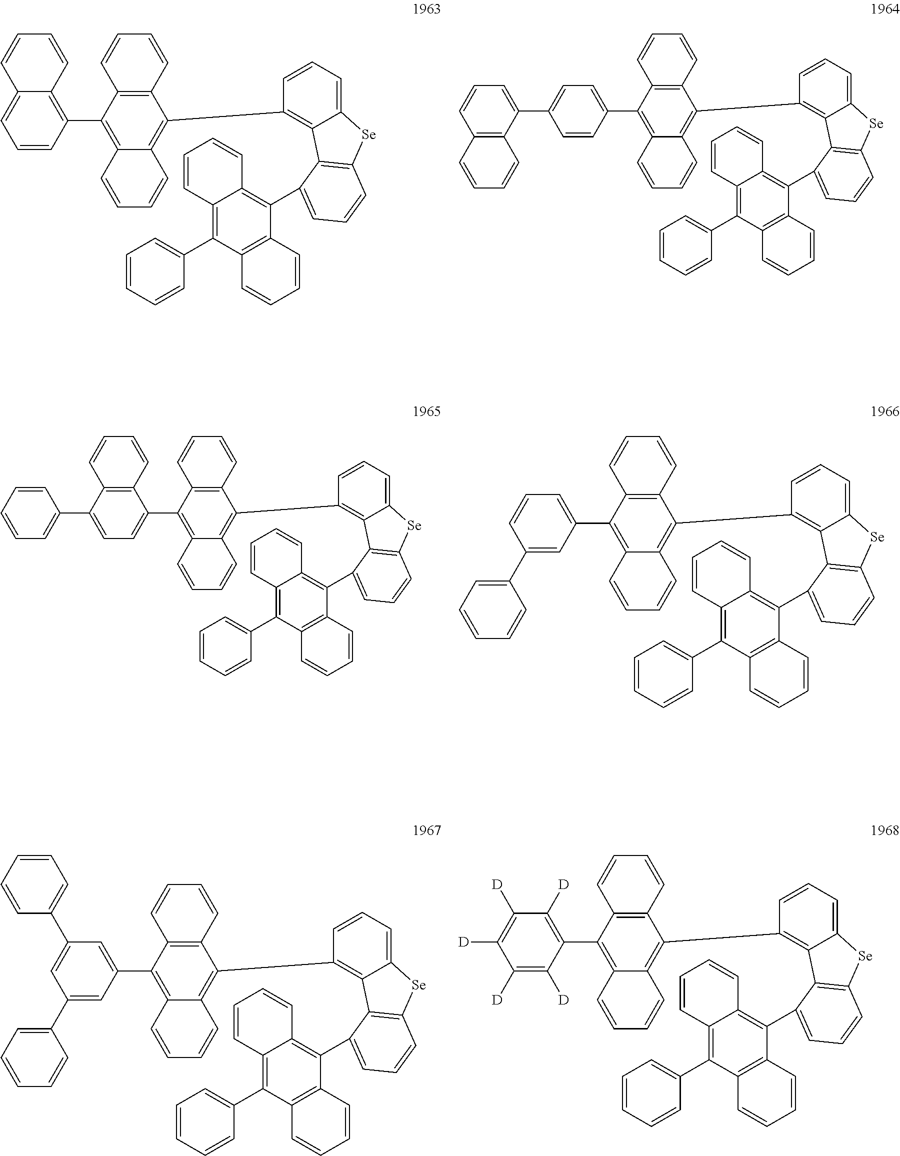

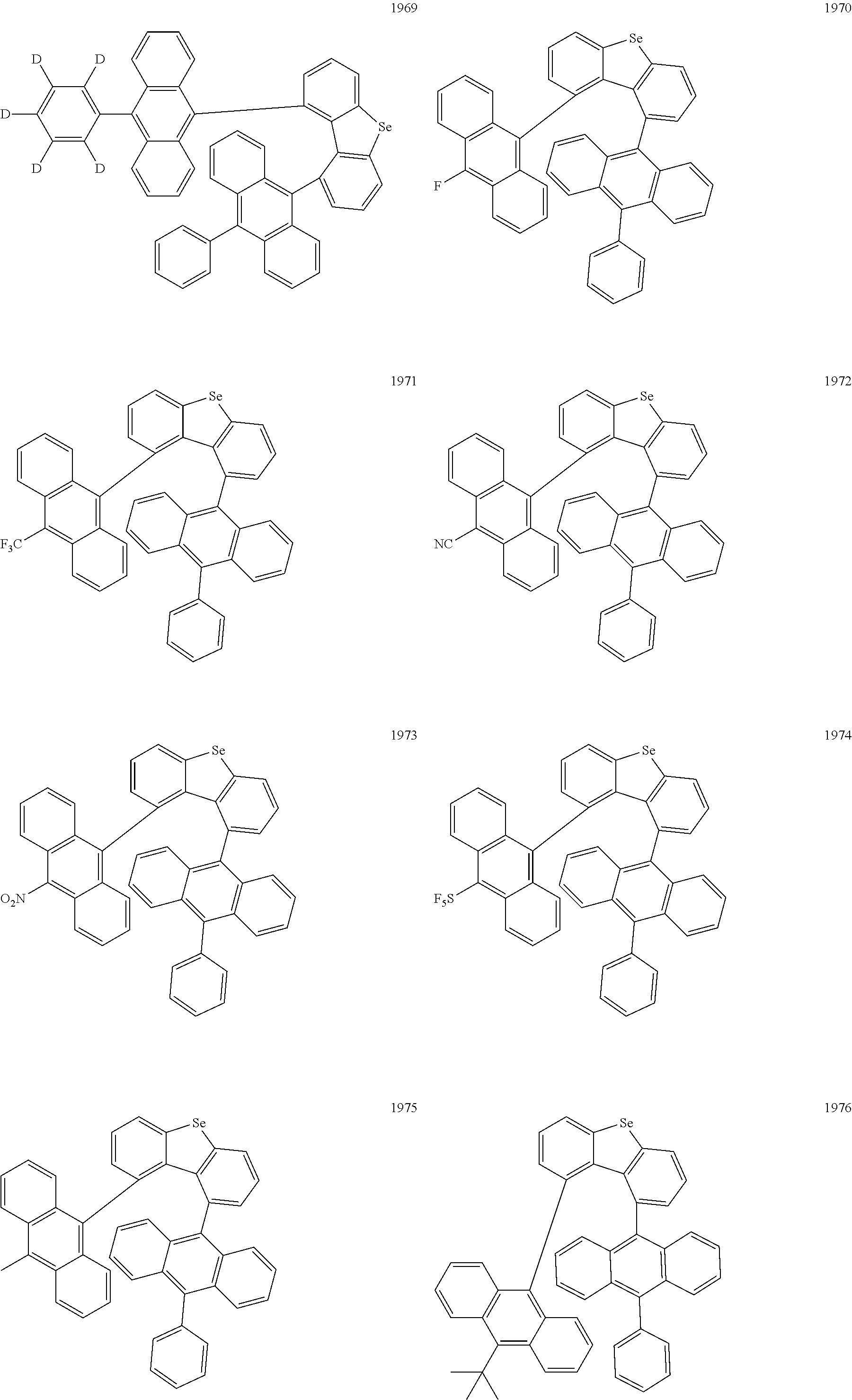

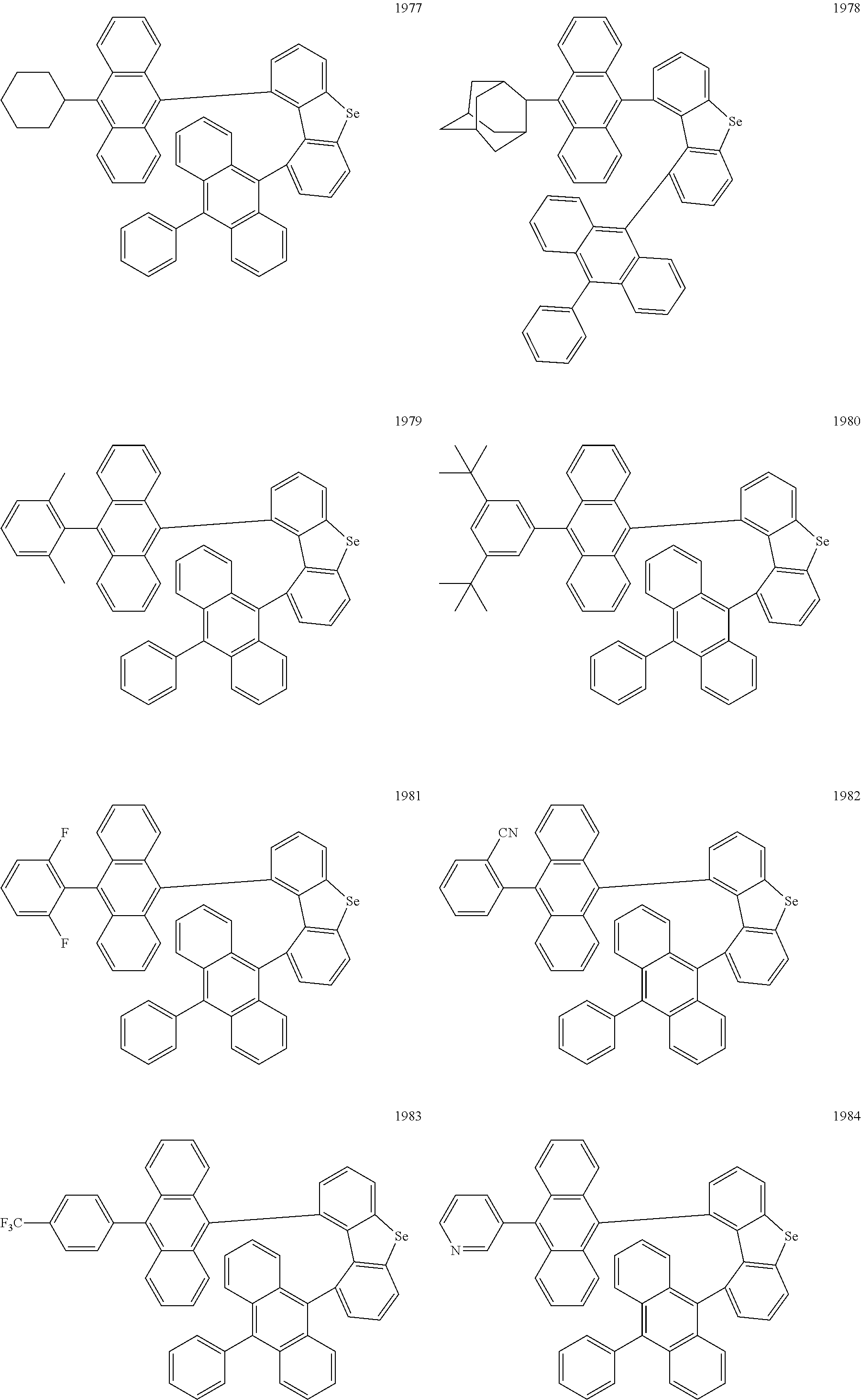

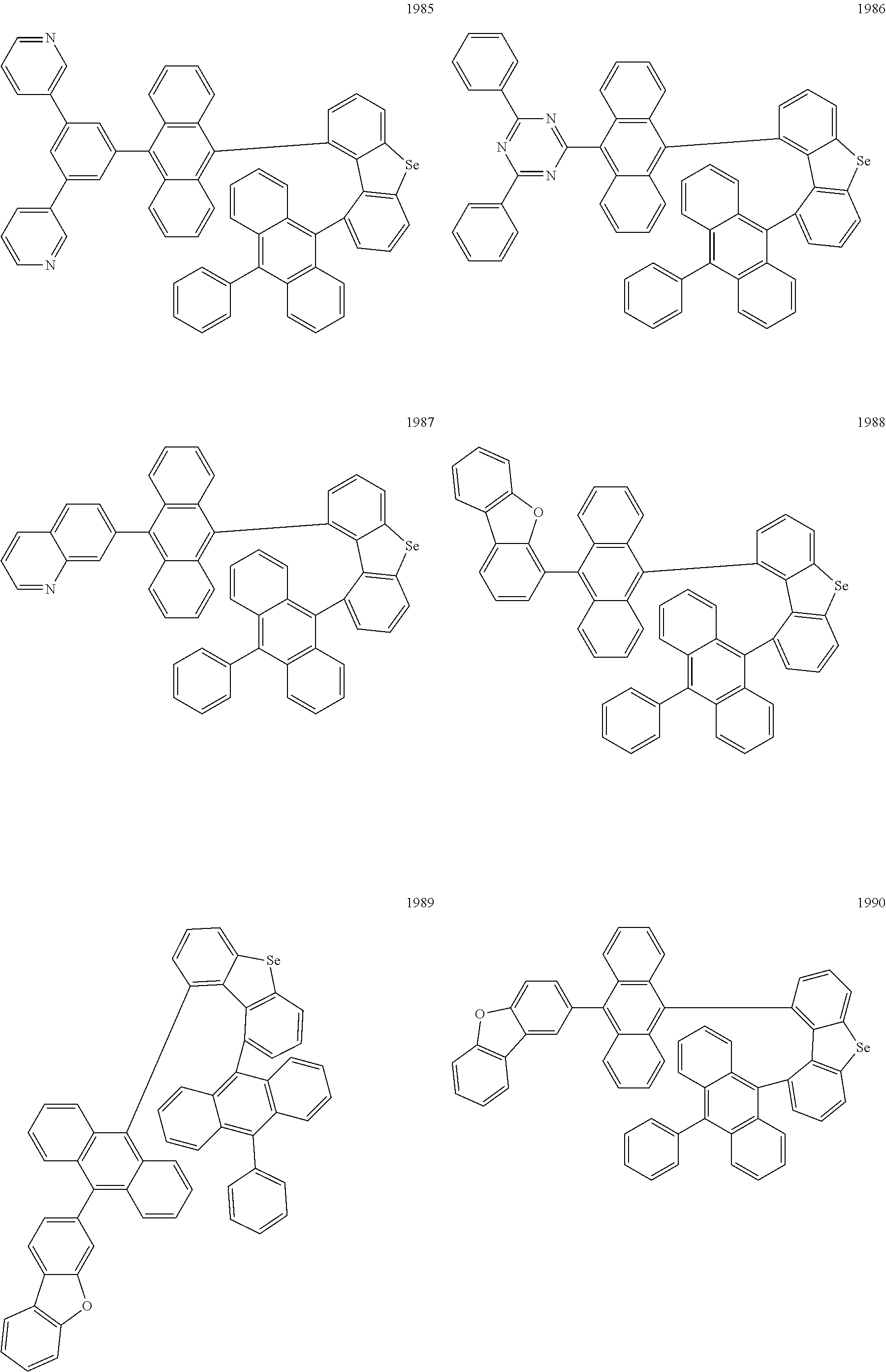

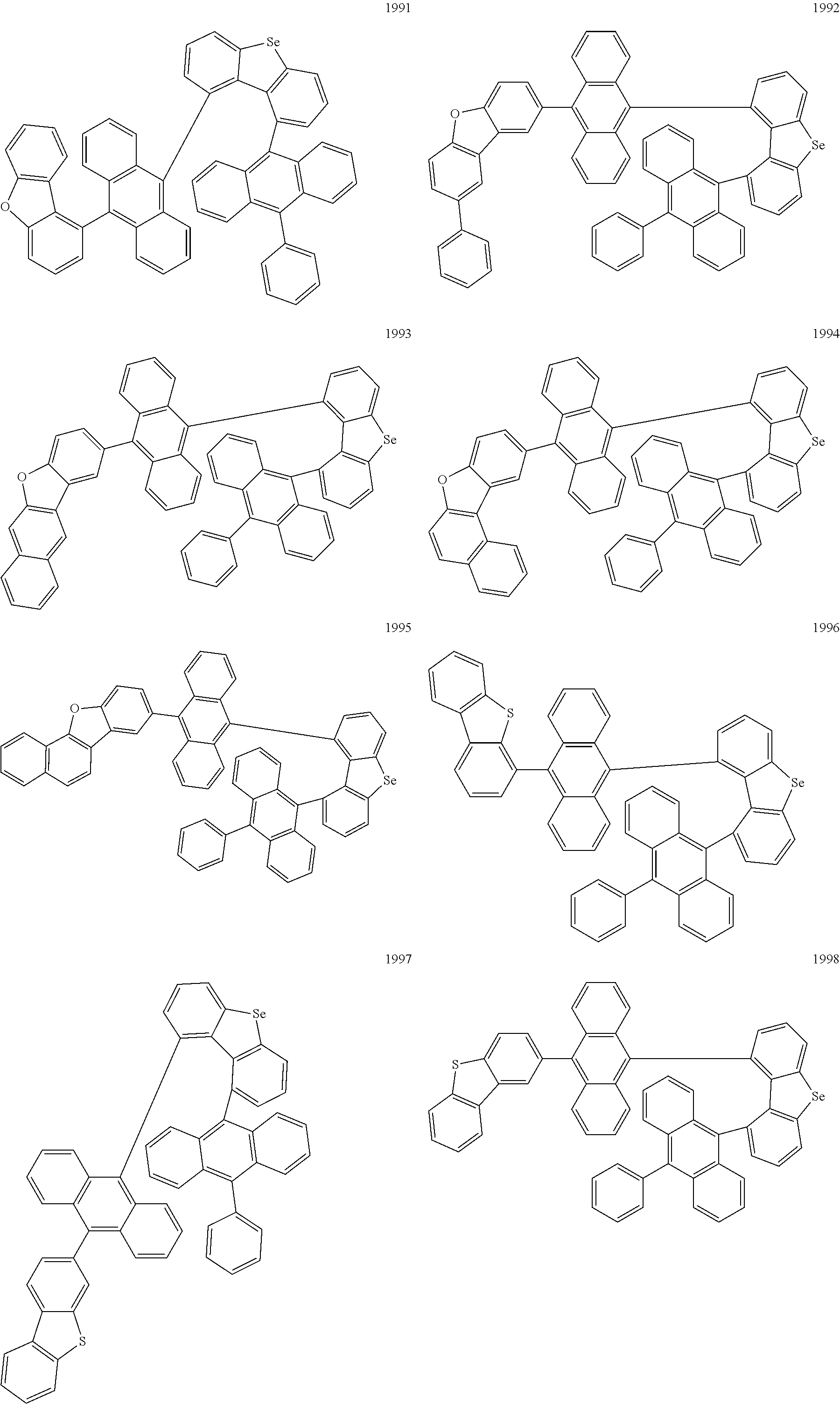

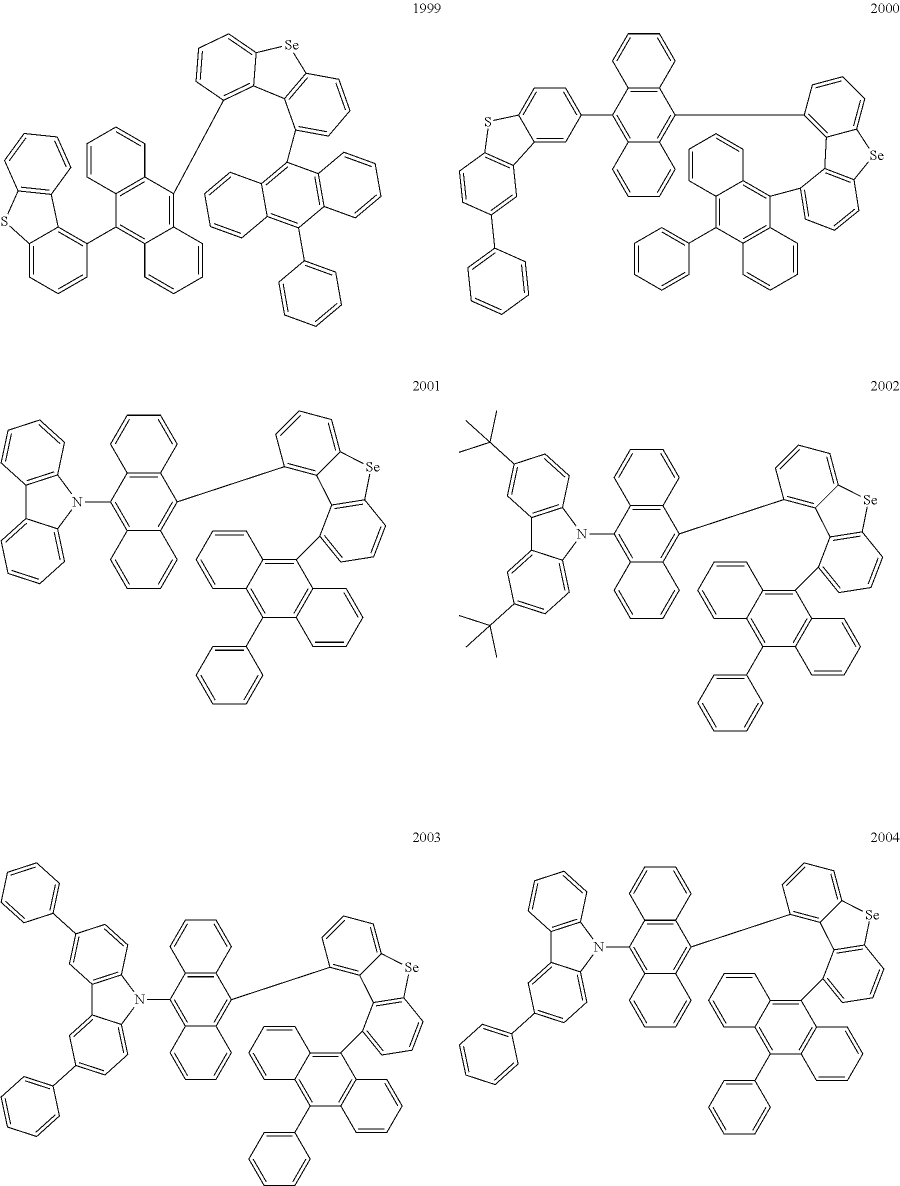

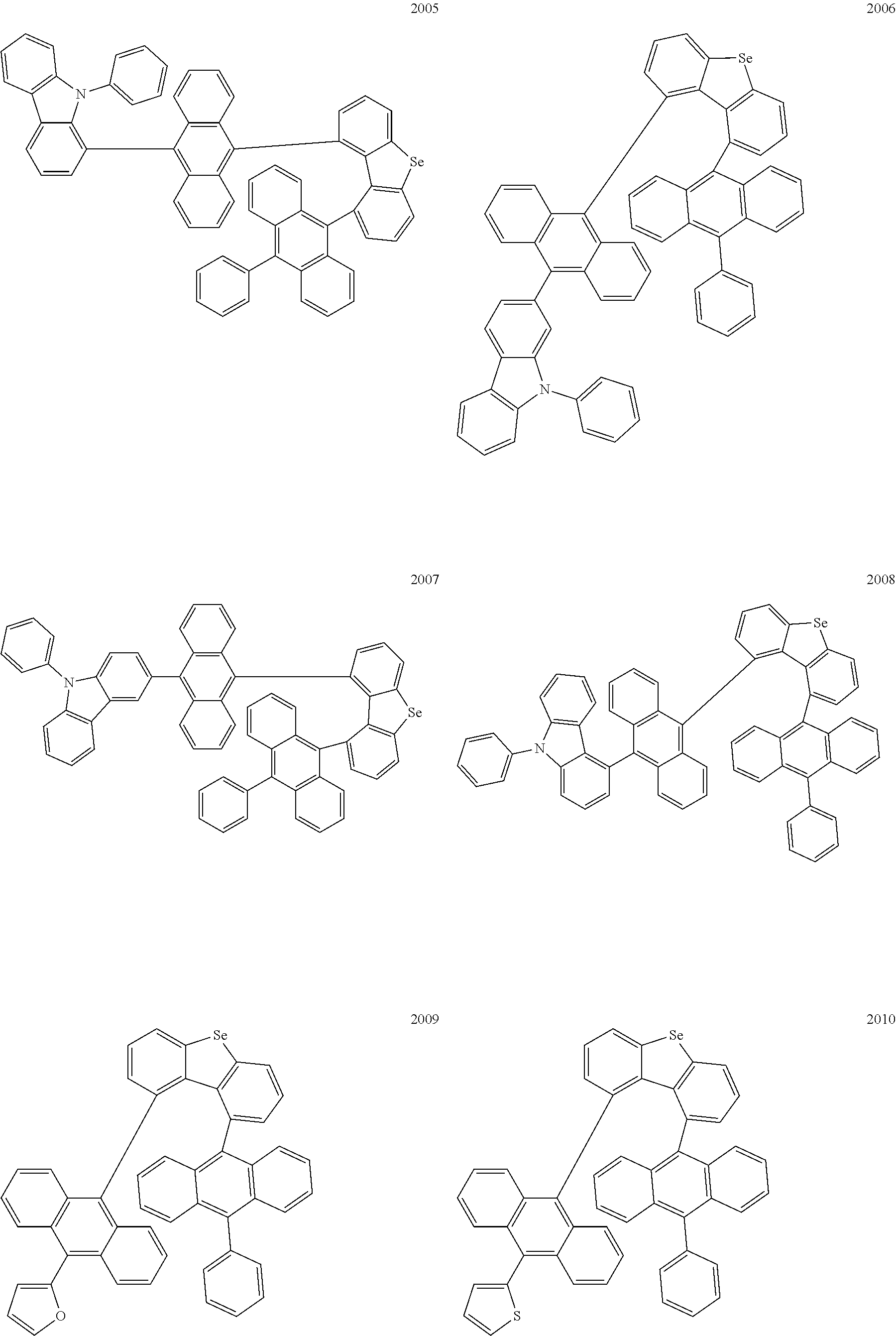

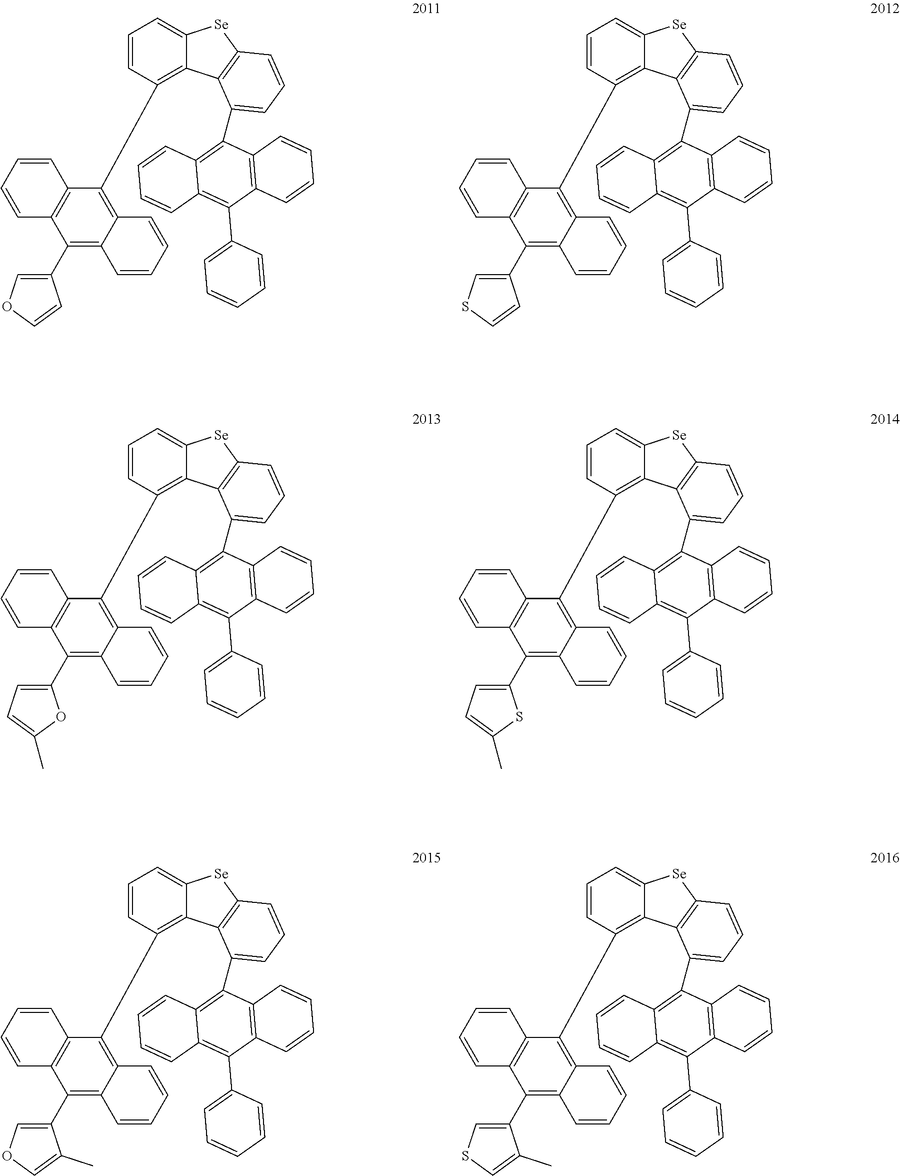

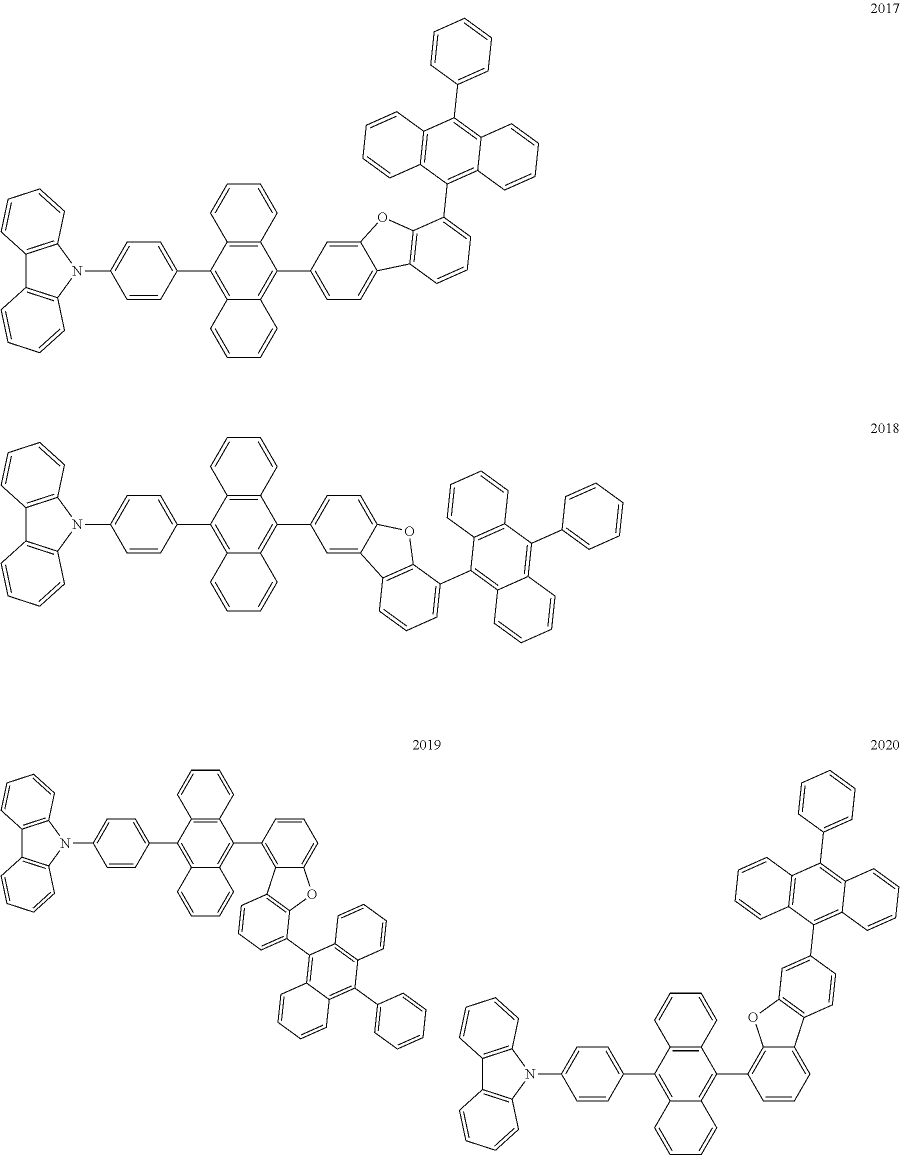

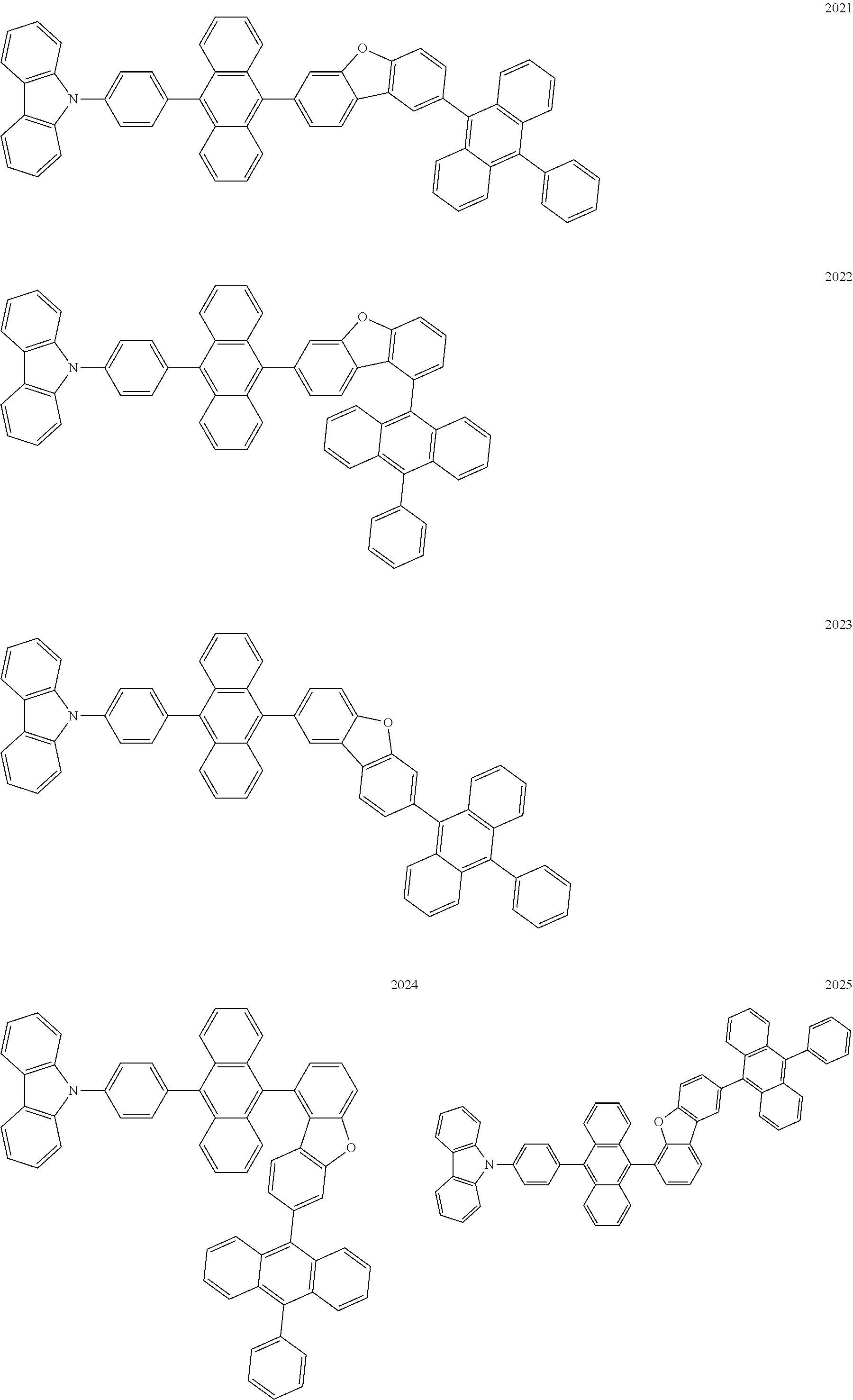

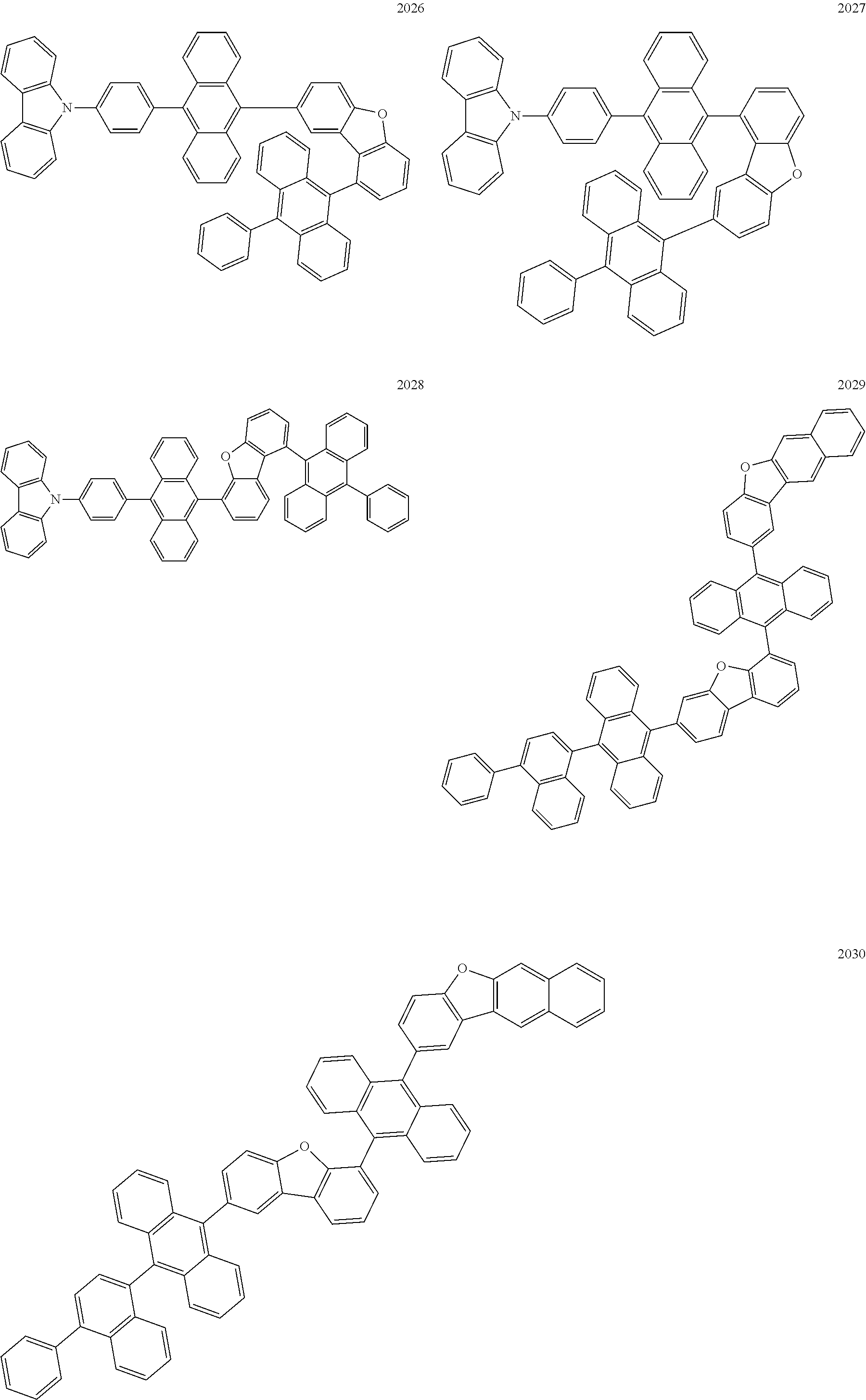

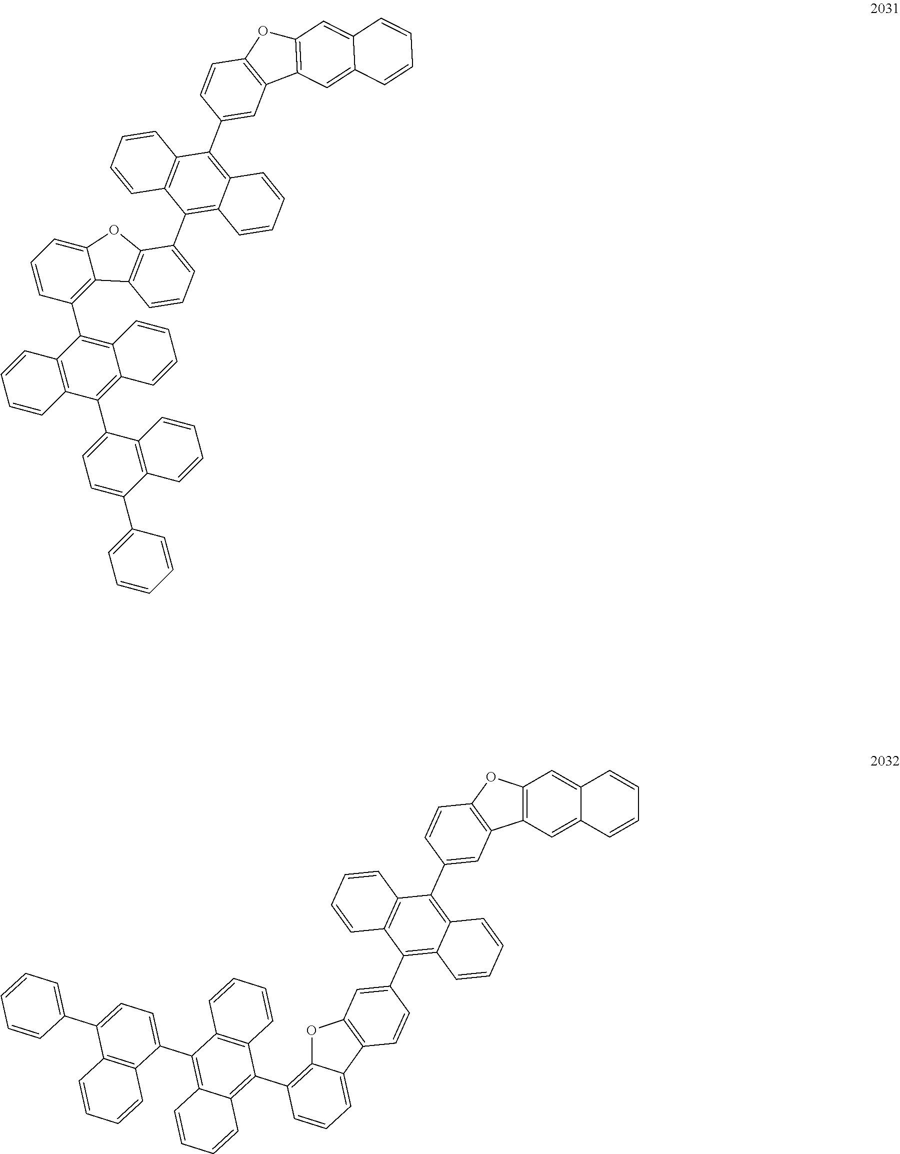 [SMILES: c1ccc(-c2ccc(-c3c4ccccc4c(-c4cccc5oc6c(-c7c8ccccc8c(-c8ccc9oc%10cc%11ccccc%11cc%10c9c8)c8ccccc78)cccc6c45)c4ccccc34)c3ccccc23)cc1 ; c1ccc(-c2ccc(-c3c4ccccc4c(-c4cccc5c4oc4cc(-c6c7ccccc7c(-c7ccc8oc9cc%10ccccc%10cc9c8c7)c7ccccc67)ccc45)c4ccccc34)c3ccccc23)cc1]

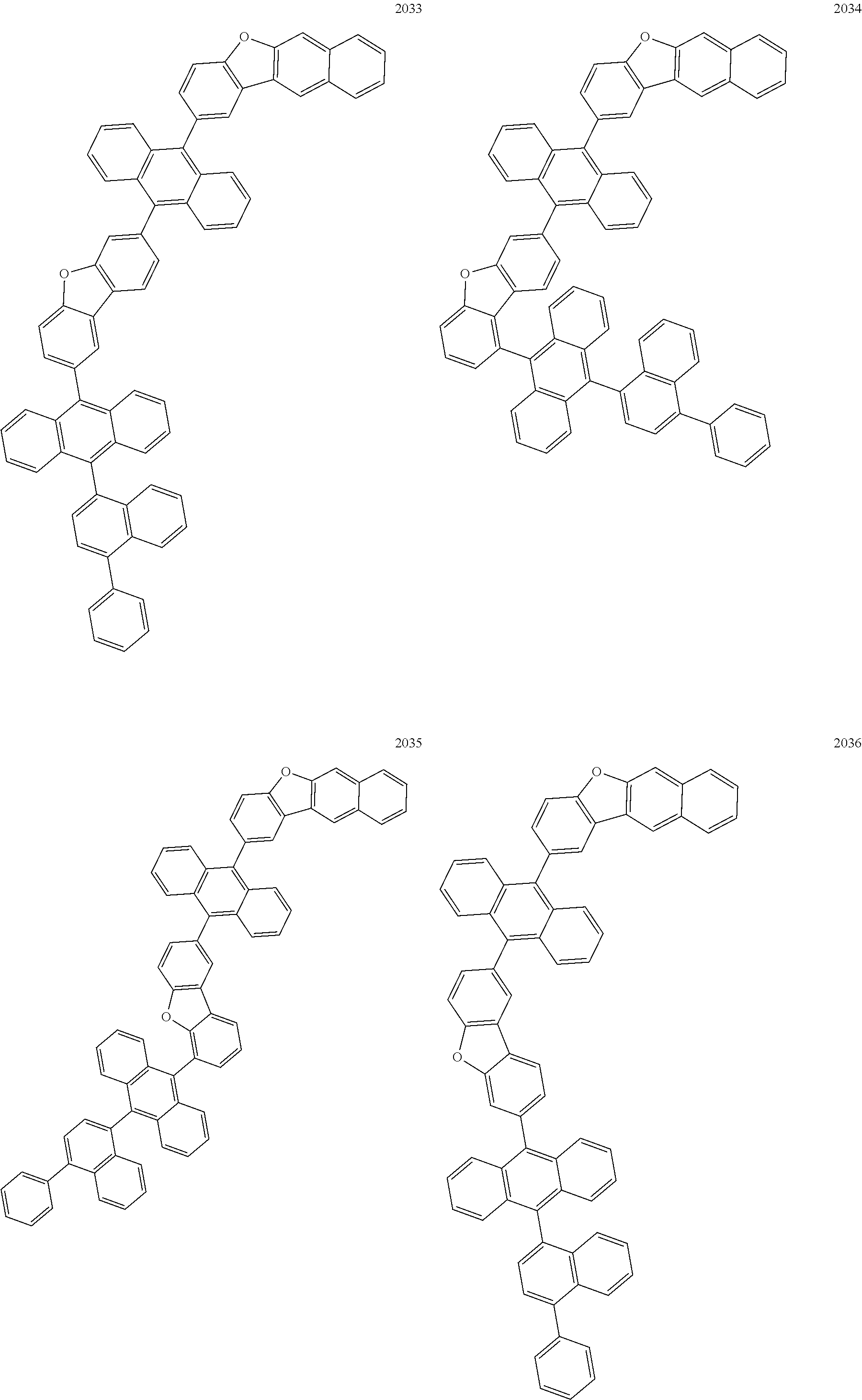

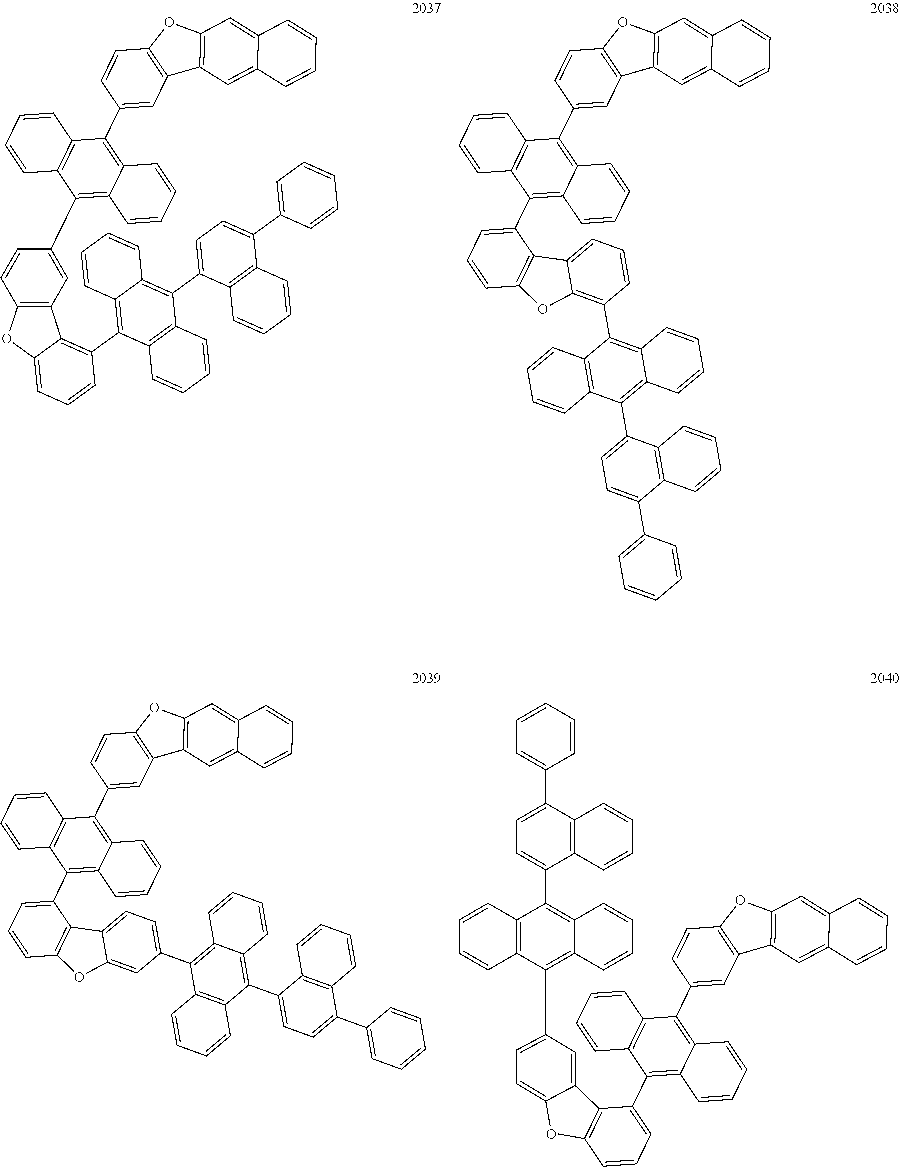

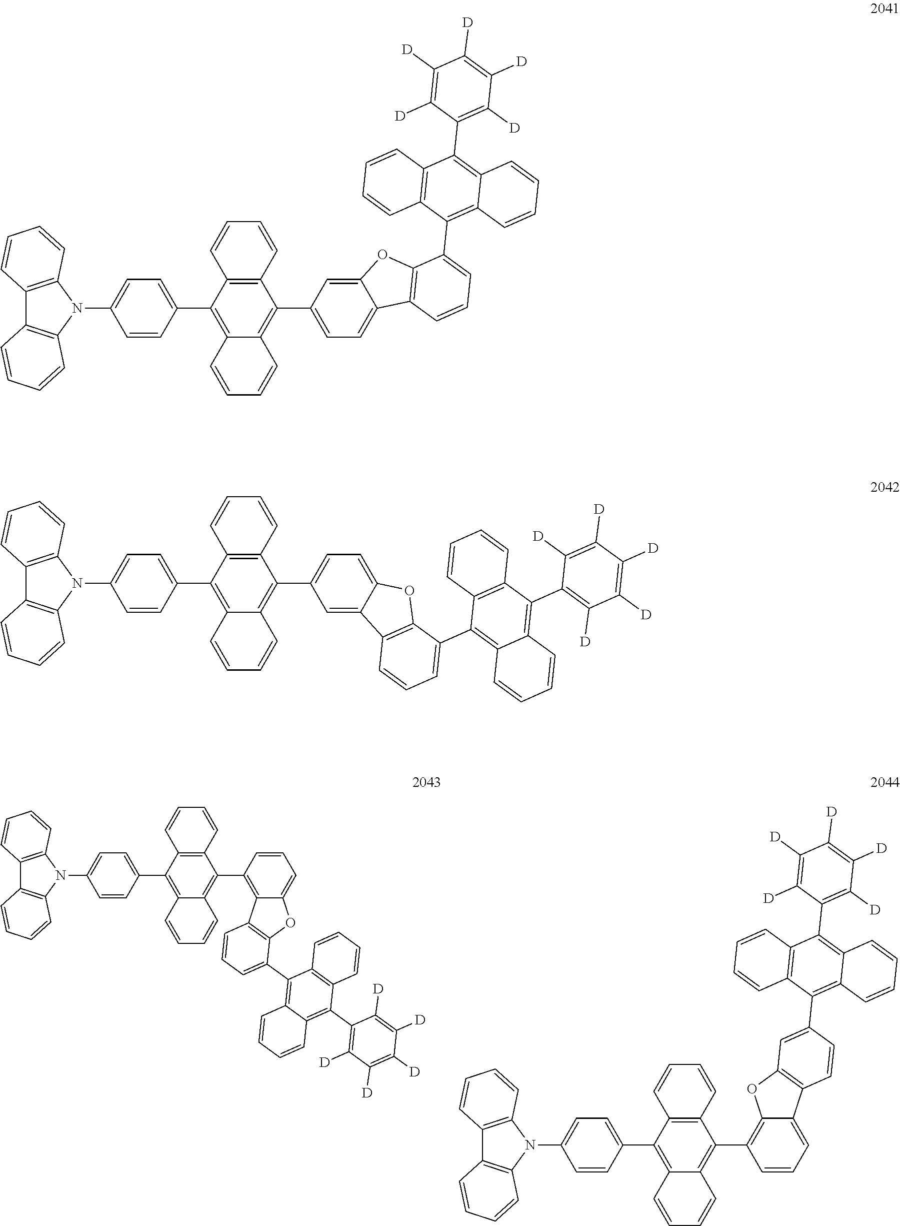

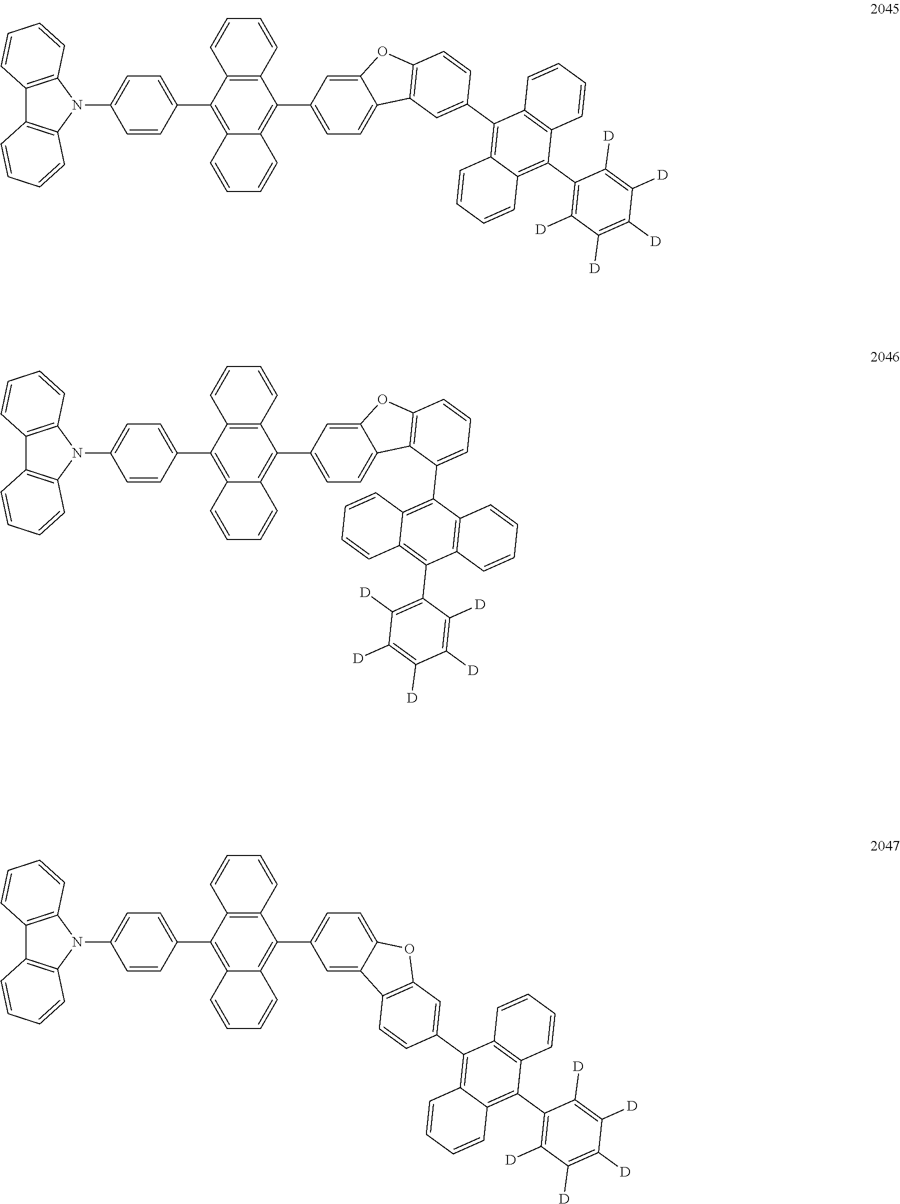 [SMILES: [2H]c1c([2H])c([2H])c(-c2c3ccccc3c(-c3ccc4oc5cc(-c6c7ccccc7c(-c7ccc(-n8c9ccccc9c9ccccc98)cc7)c7ccccc67)ccc5c4c3)c3ccccc23)c([2H])c1[2H] ; [2H]c1c([2H])c([2H])c(-c2c3ccccc3c(-c3cccc4oc5cc(-c6c7ccccc7c(-c7ccc(-n8c9ccccc9c9ccccc98)cc7)c7ccccc67)ccc5c34)c3ccccc23)c([2H])c1[2H] ; [2H]c1c([2H])c([2H])c(-c2c3ccccc3c(-c3ccc4c(c3)oc3ccc(-c5c6ccccc6c(-c6ccc(-n7c8ccccc8c8ccccc87)cc6)c6ccccc56)cc34)c3ccccc23)c([2H])c1[2H]]

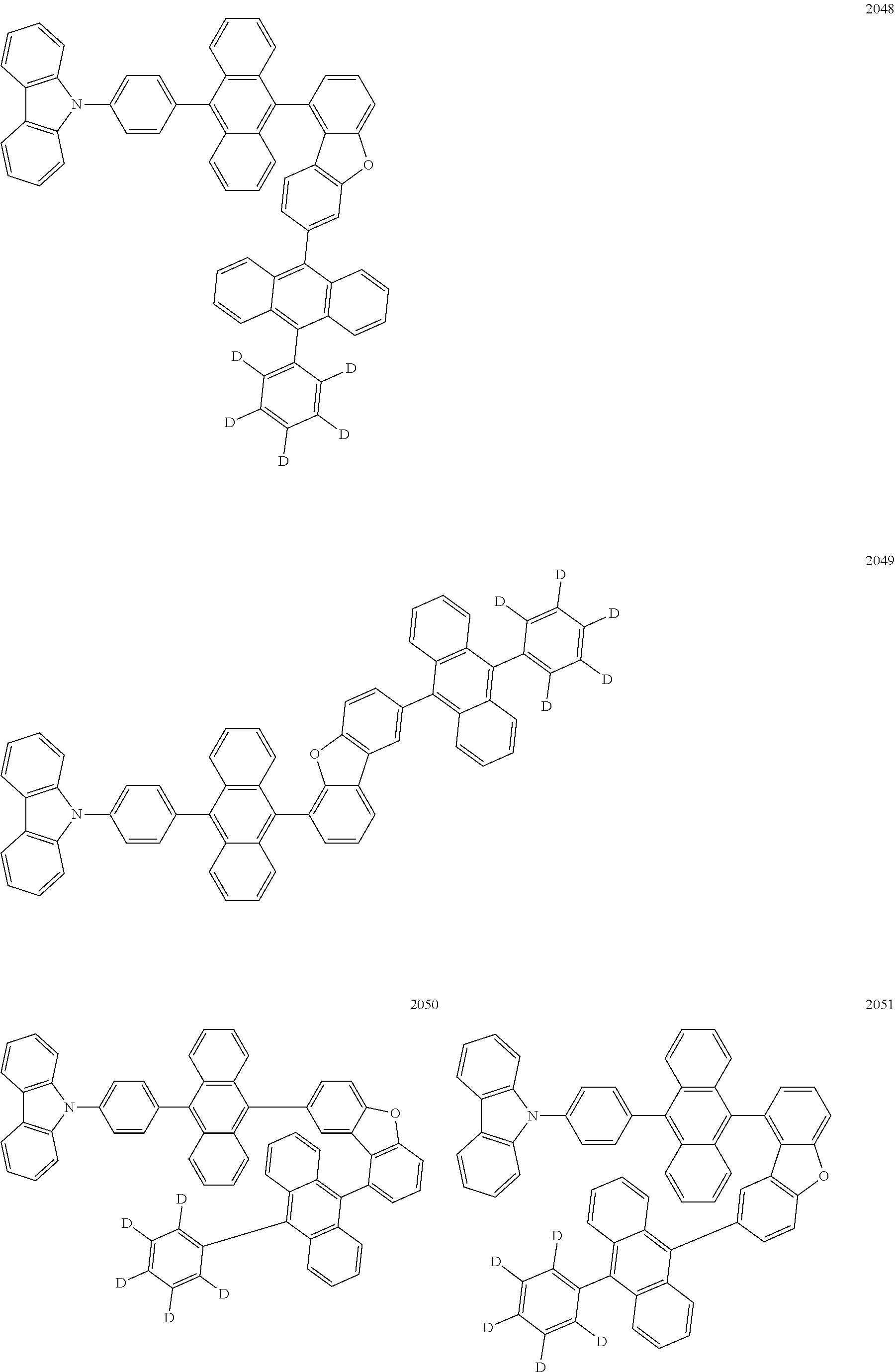

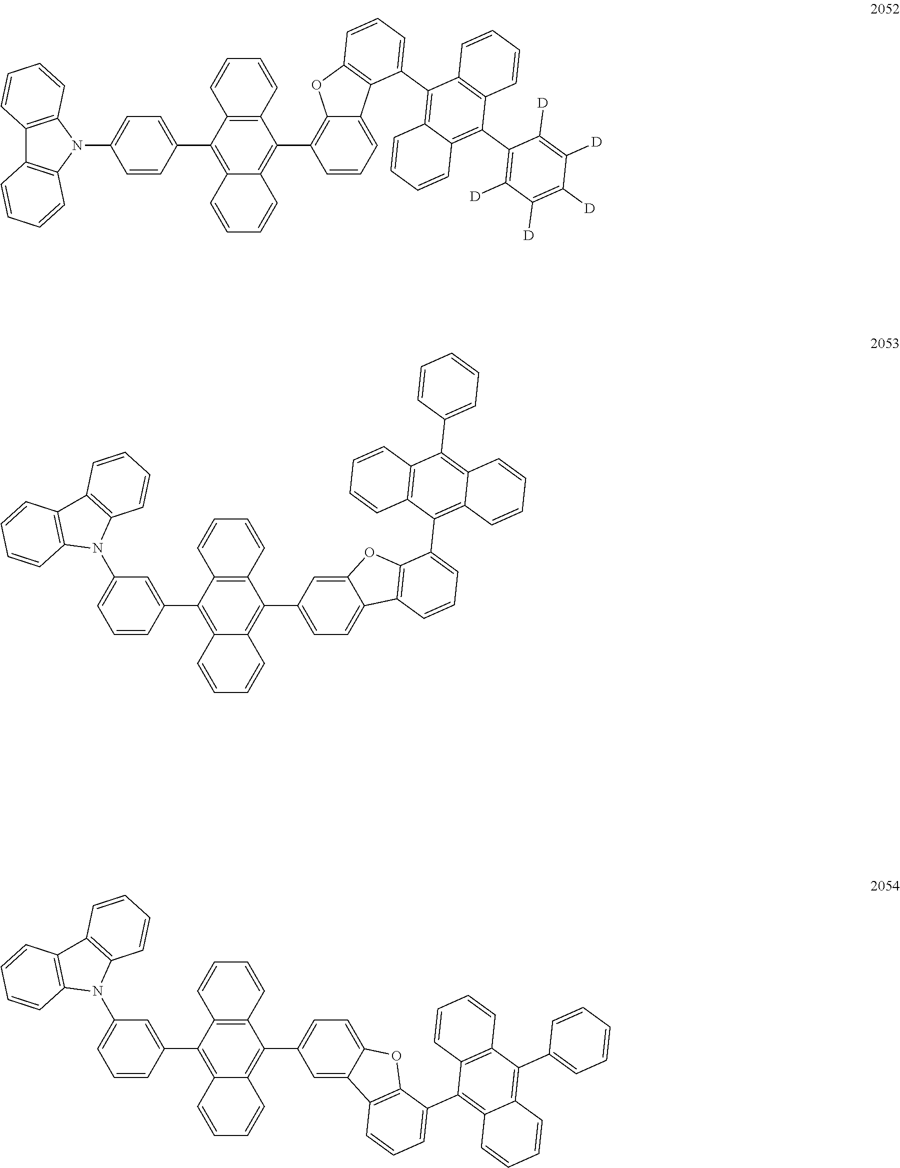 [SMILES: [2H]c1c([2H])c([2H])c(-c2c3ccccc3c(-c3cccc4oc5c(-c6c7ccccc7c(-c7ccc(-n8c9ccccc9c9ccccc98)cc7)c7ccccc67)cccc5c34)c3ccccc23)c([2H])c1[2H] ; c1ccc(-c2c3ccccc3c(-c3cccc4c3oc3cc(-c5c6ccccc6c(-c6cccc(-n7c8ccccc8c8ccccc87)c6)c6ccccc56)ccc34)c3ccccc23)cc1 ; c1ccc(-c2c3ccccc3c(-c3cccc4c3oc3ccc(-c5c6ccccc6c(-c6cccc(-n7c8ccccc8c8ccccc87)c6)c6ccccc56)cc34)c3ccccc23)cc1]

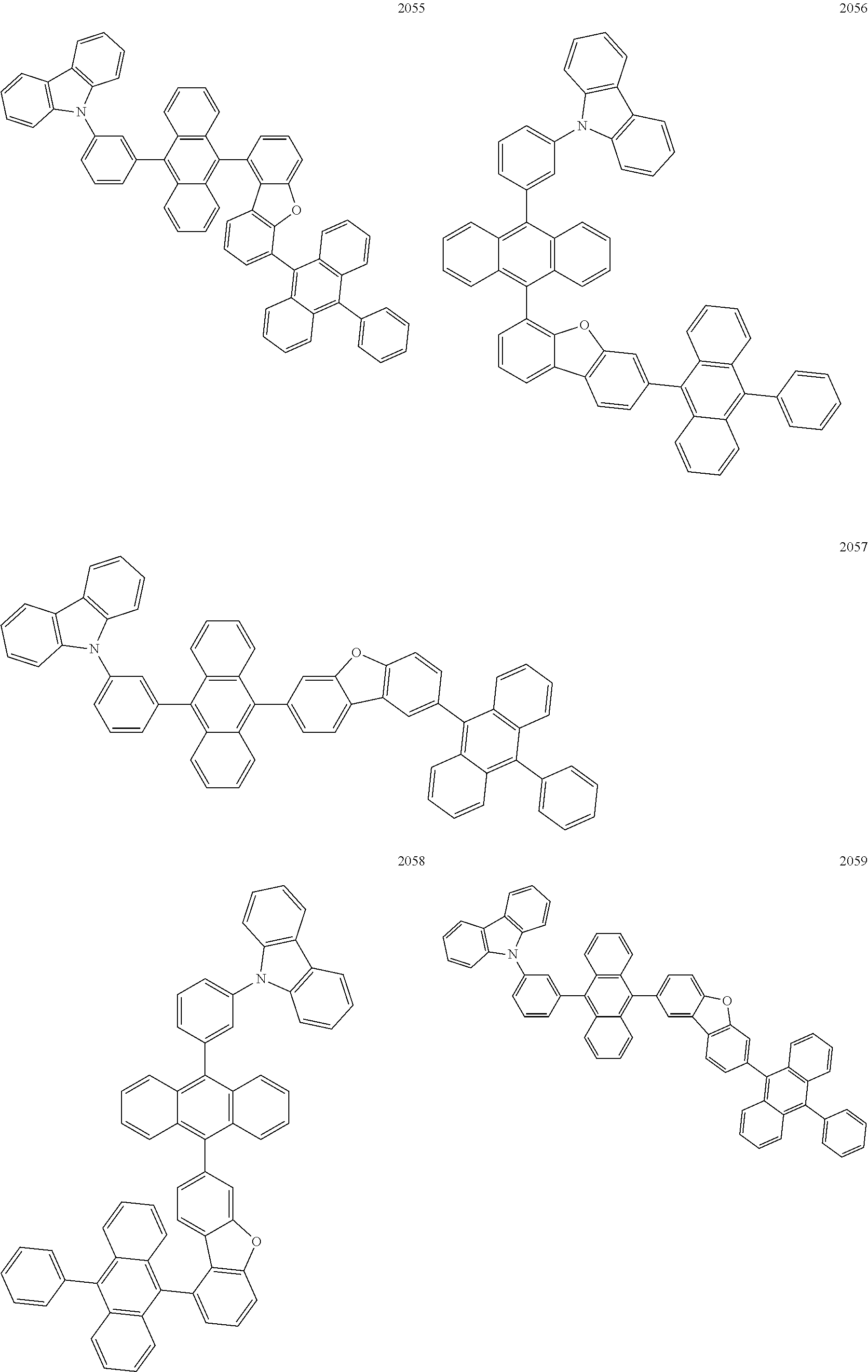

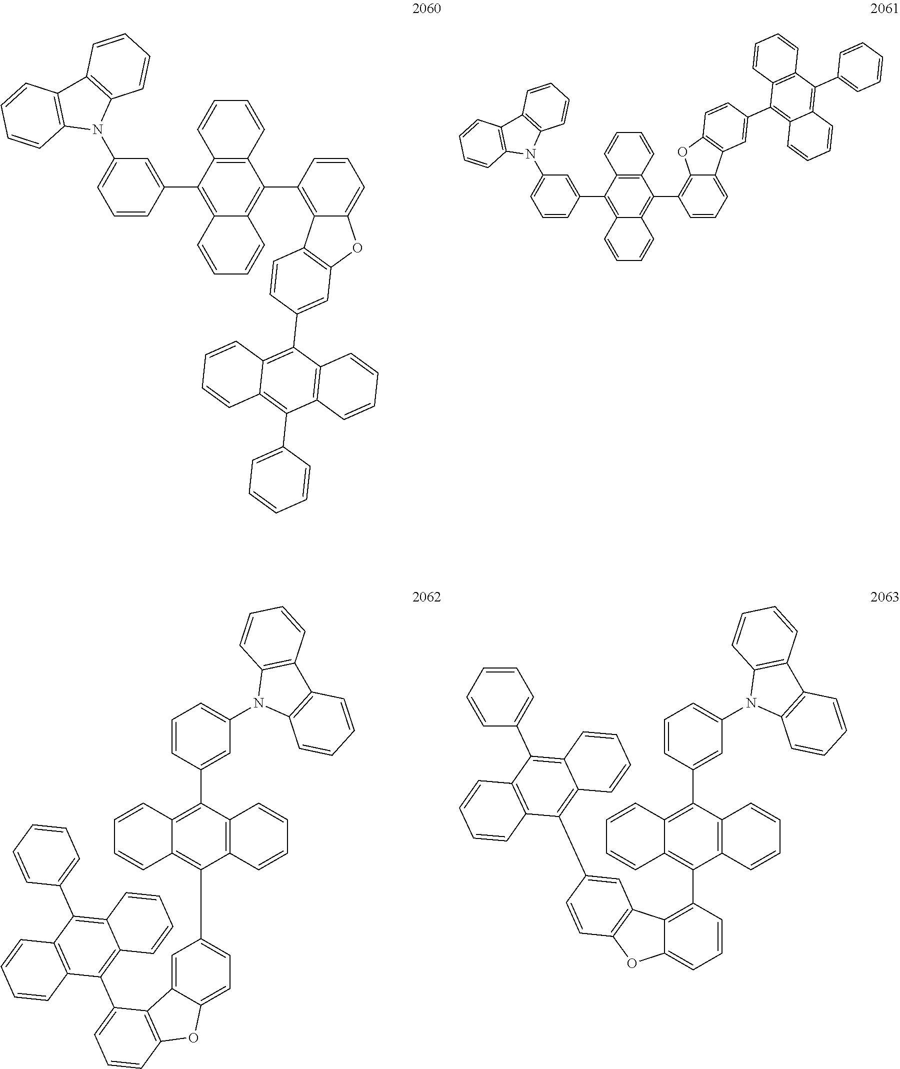 [SMILES: c1ccc(-c2c3ccccc3c(-c3ccc4c(c3)oc3cccc(-c5c6ccccc6c(-c6cccc(-n7c8ccccc8c8ccccc87)c6)c6ccccc56)c34)c3ccccc23)cc1 ; c1ccc(-c2c3ccccc3c(-c3ccc4oc5c(-c6c7ccccc7c(-c7cccc(-n8c9ccccc9c9ccccc98)c7)c7ccccc67)cccc5c4c3)c3ccccc23)cc1 ; c1ccc(-c2c3ccccc3c(-c3cccc4oc5ccc(-c6c7ccccc7c(-c7cccc(-n8c9ccccc9c9ccccc98)c7)c7ccccc67)cc5c34)c3ccccc23)cc1 ; c1ccc(-c2c3ccccc3c(-c3ccc4oc5cccc(-c6c7ccccc7c(-c7cccc(-n8c9ccccc9c9ccccc98)c7)c7ccccc67)c5c4c3)c3ccccc23)cc1]

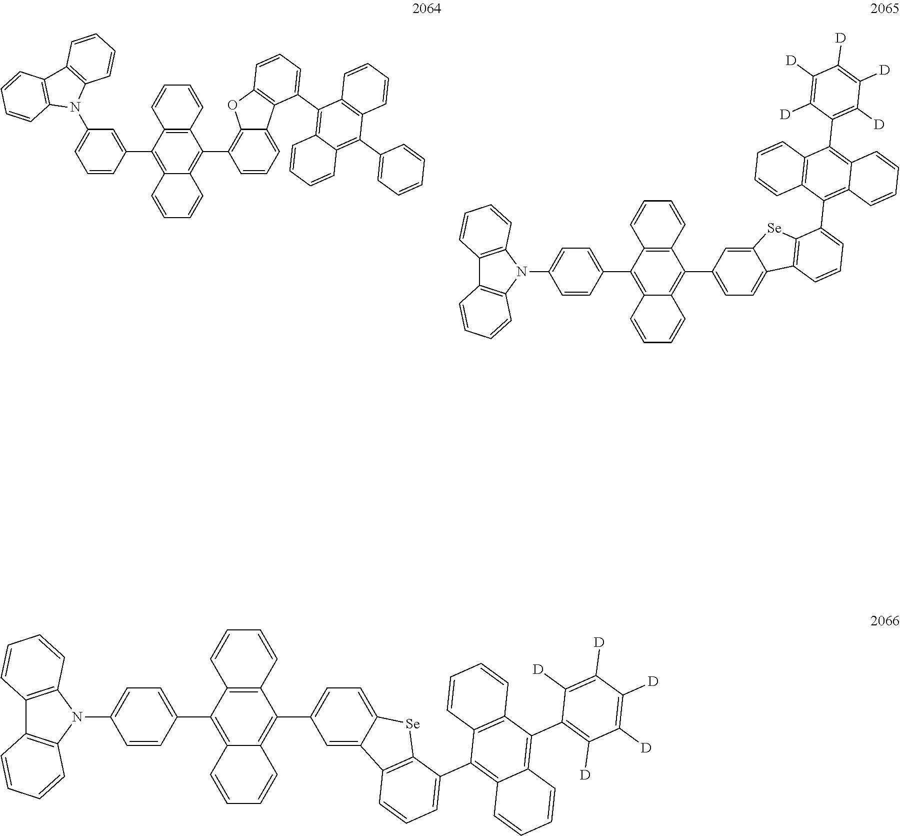 [SMILES: c1ccc(-c2c3ccccc3c(-c3cccc4oc5c(-c6c7ccccc7c(-c7cccc(-n8c9ccccc9c9ccccc98)c7)c7ccccc67)cccc5c34)c3ccccc23)cc1 ; [2H]c1c([2H])c([2H])c(-c2c3ccccc3c(-c3cccc4c3[se]c3cc(-c5c6ccccc6c(-c6ccc(-n7c8ccccc8c8ccccc87)cc6)c6ccccc56)ccc34)c3ccccc23)c([2H])c1[2H] ; [2H]c1c([2H])c([2H])c(-c2c3ccccc3c(-c3cccc4c3[se]c3ccc(-c5c6ccccc6c(-c6ccc(-n7c8ccccc8c8ccccc87)cc6)c6ccccc56)cc34)c3ccccc23)c([2H])c1[2H]]

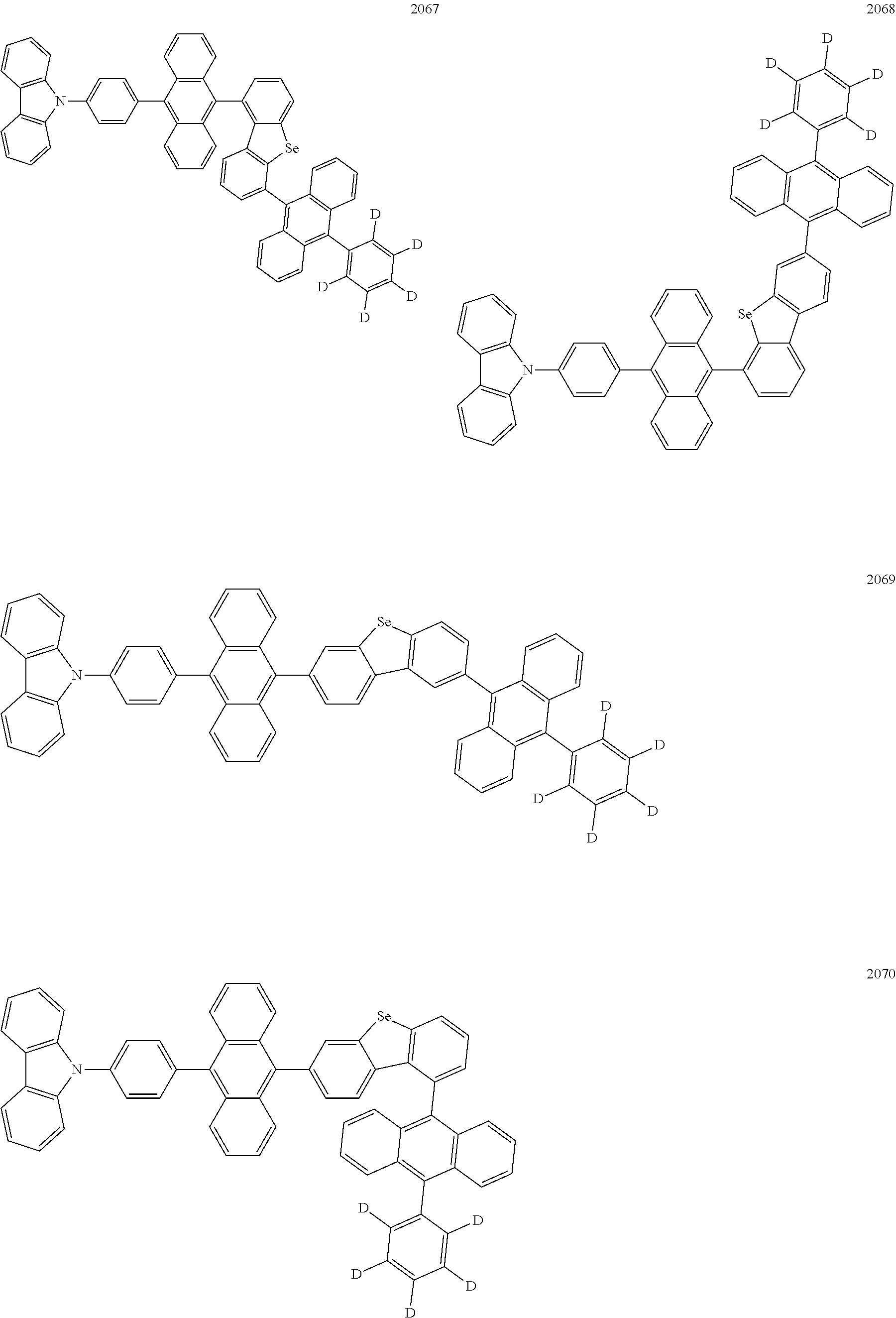

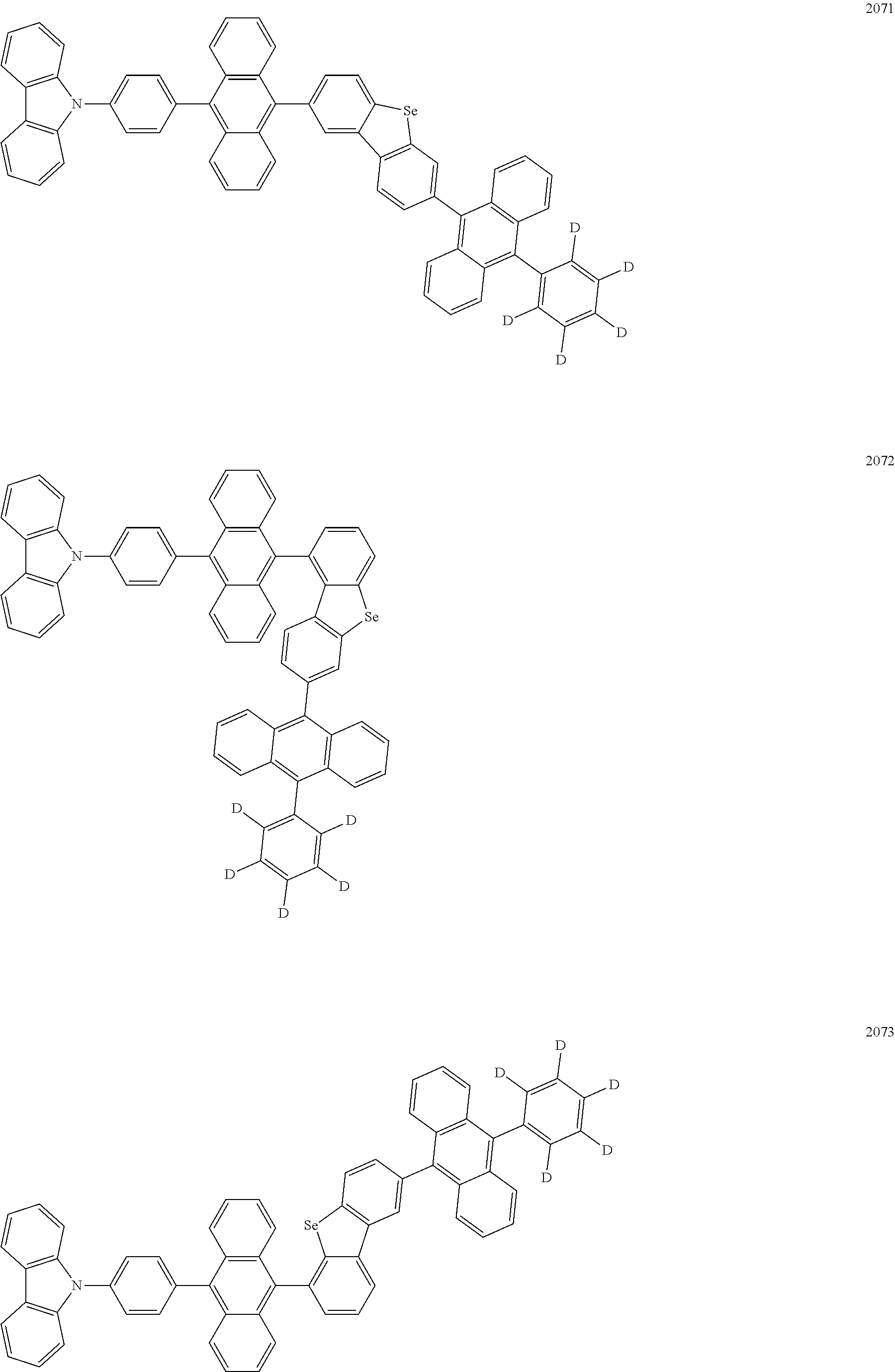 [SMILES: [2H]c1c([2H])c([2H])c(-c2c3ccccc3c(-c3ccc4c(c3)[se]c3ccc(-c5c6ccccc6c(-c6ccc(-n7c8ccccc8c8ccccc87)cc6)c6ccccc56)cc34)c3ccccc23)c([2H])c1[2H] ; [2H]c1c([2H])c([2H])c(-c2c3ccccc3c(-c3ccc4c(c3)[se]c3cccc(-c5c6ccccc6c(-c6ccc(-n7c8ccccc8c8ccccc87)cc6)c6ccccc56)c34)c3ccccc23)c([2H])c1[2H] ; [2H]c1c([2H])c([2H])c(-c2c3ccccc3c(-c3ccc4[se]c5c(-c6c7ccccc7c(-c7ccc(-n8c9ccccc9c9ccccc98)cc7)c7ccccc67)cccc5c4c3)c3ccccc23)c([2H])c1[2H]]

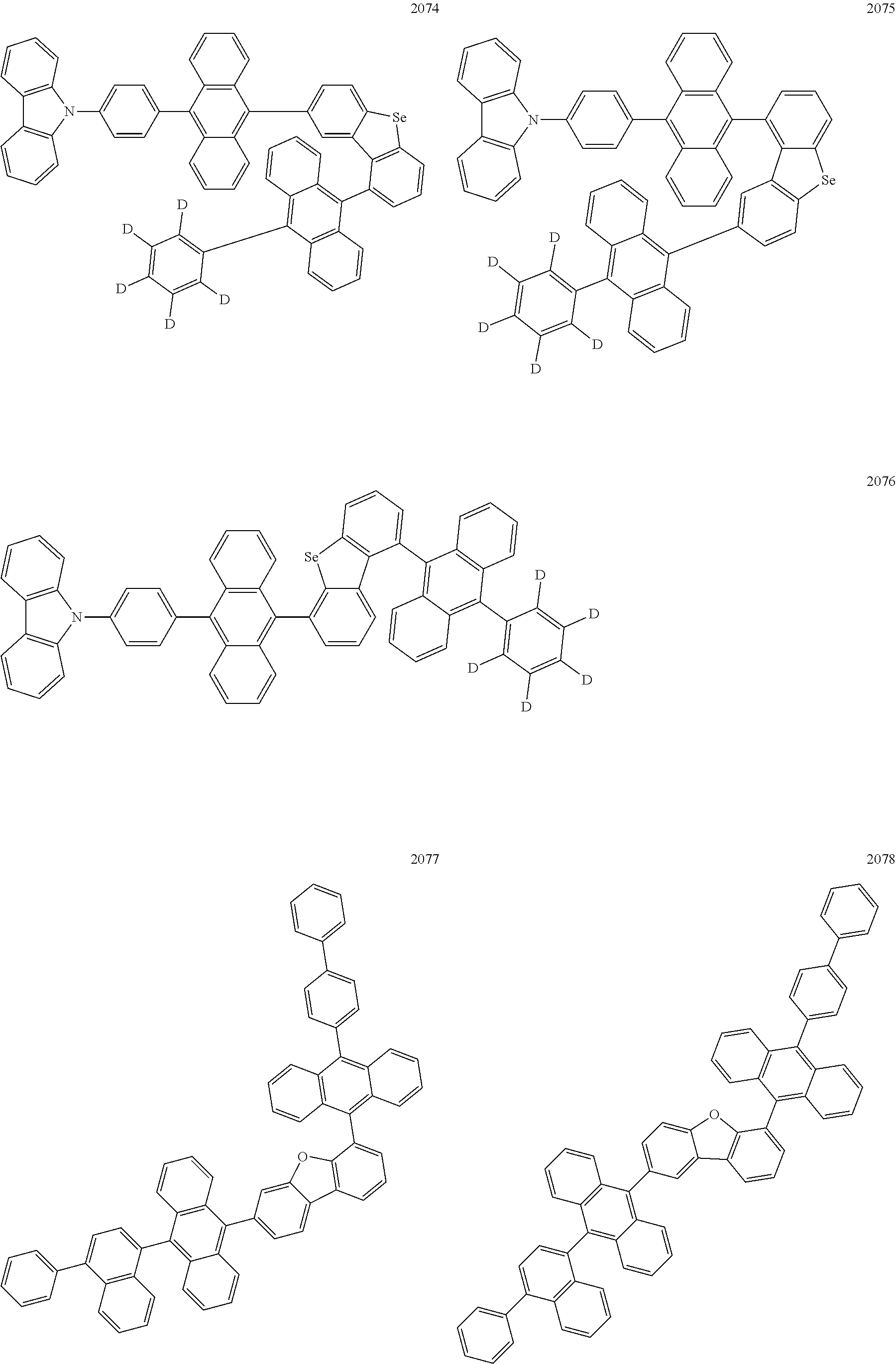

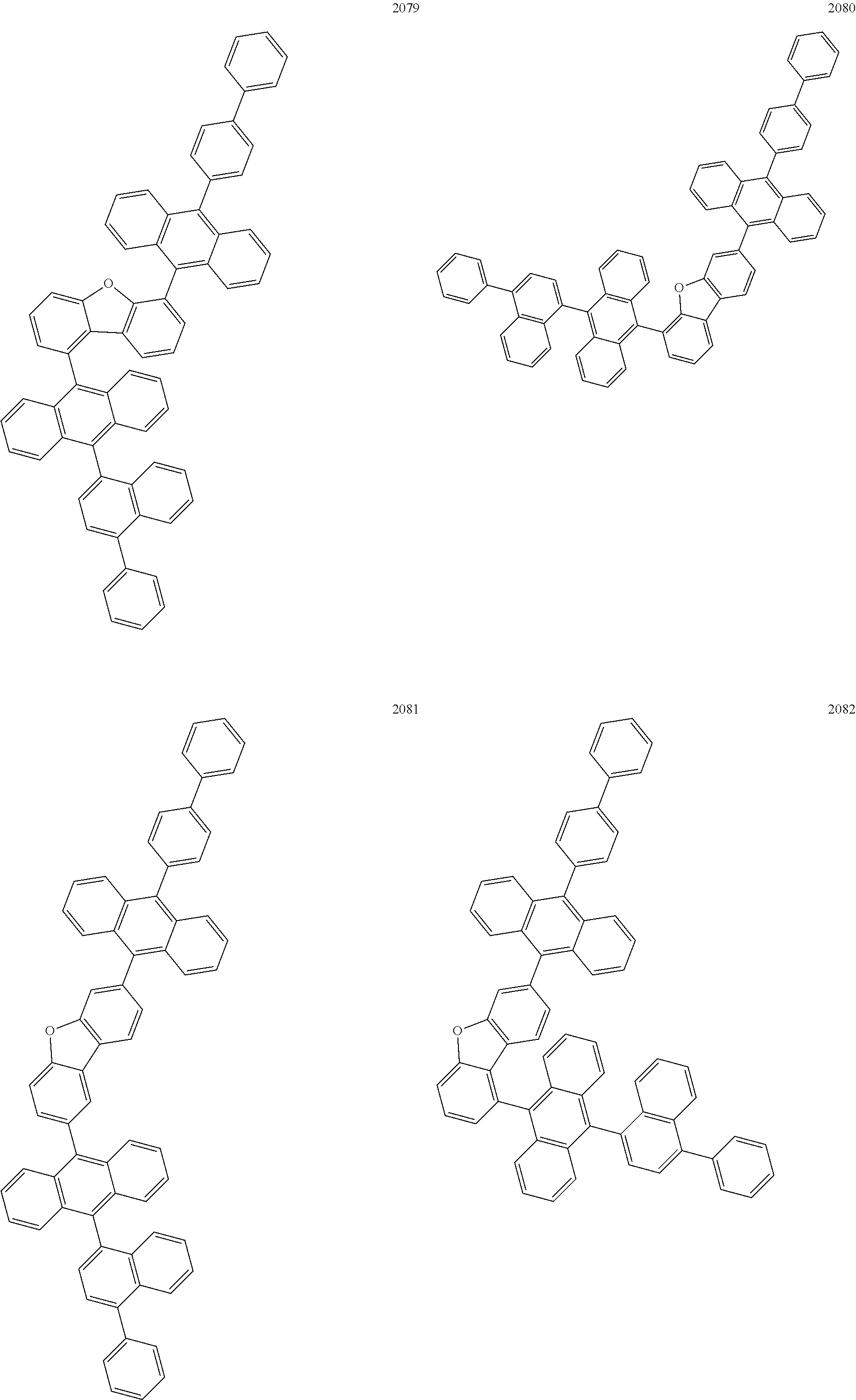 [SMILES: c1ccc(-c2ccc(-c3c4ccccc4c(-c4cccc5c4oc4cccc(-c6c7ccccc7c(-c7ccc(-c8ccccc8)c8ccccc78)c7ccccc67)c45)c4ccccc34)cc2)cc1 ; c1ccc(-c2ccc(-c3c4ccccc4c(-c4ccc5c(c4)oc4c(-c6c7ccccc7c(-c7ccc(-c8ccccc8)c8ccccc78)c7ccccc67)cccc45)c4ccccc34)cc2)cc1 ; c1ccc(-c2ccc(-c3c4ccccc4c(-c4ccc5c(c4)oc4ccc(-c6c7ccccc7c(-c7ccc(-c8ccccc8)c8ccccc78)c7ccccc67)cc45)c4ccccc34)cc2)cc1 ; c1ccc(-c2ccc(-c3c4ccccc4c(-c4ccc5c(c4)oc4cccc(-c6c7ccccc7c(-c7ccc(-c8ccccc8)c8ccccc78)c7ccccc67)c45)c4ccccc34)cc2)cc1]

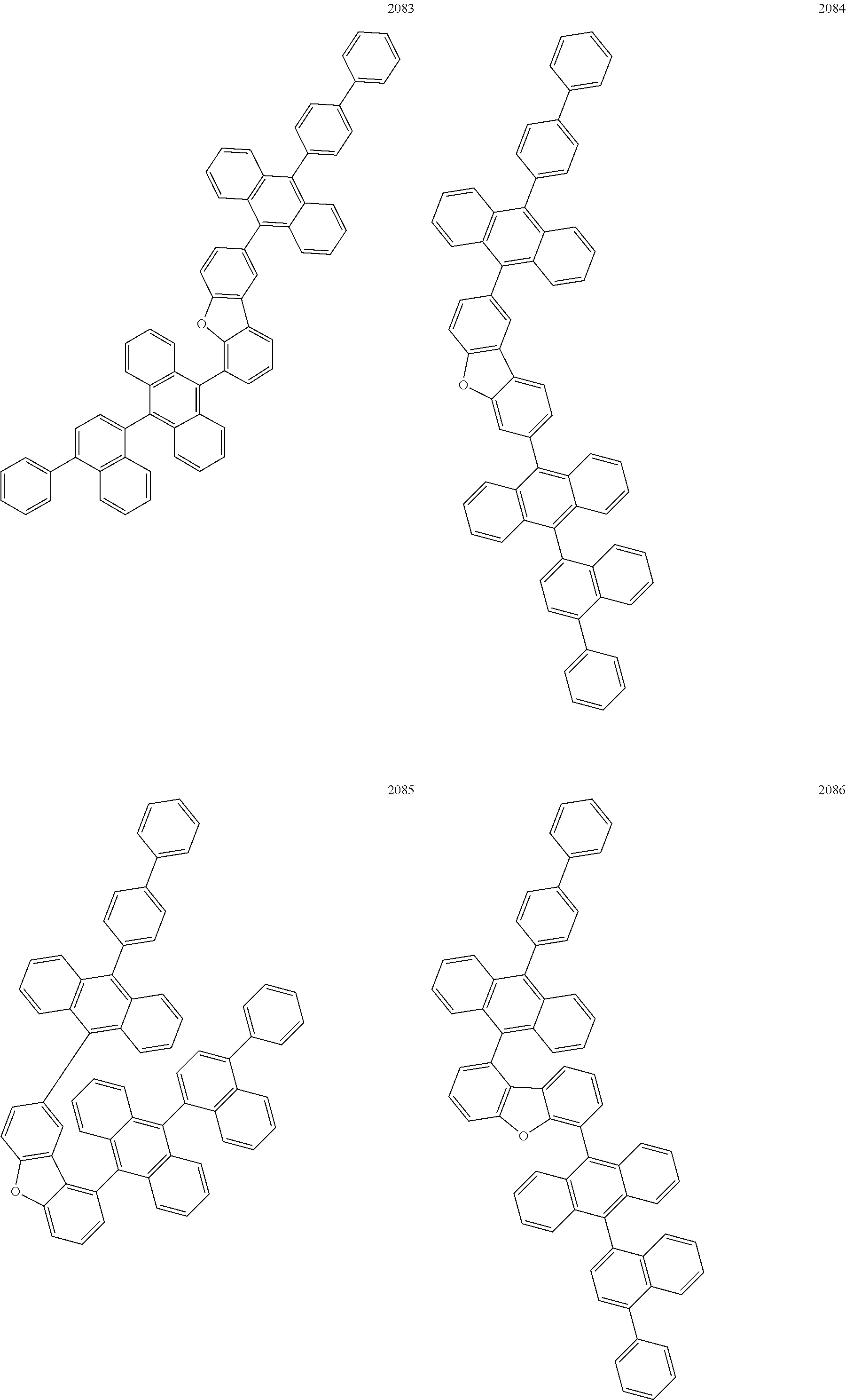 [SMILES: c1ccc(-c2ccc(-c3c4ccccc4c(-c4ccc5oc6c(-c7c8ccccc8c(-c8ccc(-c9ccccc9)c9ccccc89)c8ccccc78)cccc6c5c4)c4ccccc34)cc2)cc1 ; c1ccc(-c2ccc(-c3c4ccccc4c(-c4ccc5oc6cc(-c7c8ccccc8c(-c8ccc(-c9ccccc9)c9ccccc89)c8ccccc78)ccc6c5c4)c4ccccc34)cc2)cc1 ; c1ccc(-c2ccc(-c3c4ccccc4c(-c4ccc5oc6cccc(-c7c8ccccc8c(-c8ccc(-c9ccccc9)c9ccccc89)c8ccccc78)c6c5c4)c4ccccc34)cc2)cc1 ; c1ccc(-c2ccc(-c3c4ccccc4c(-c4cccc5oc6c(-c7c8ccccc8c(-c8ccc(-c9ccccc9)c9ccccc89)c8ccccc78)cccc6c45)c4ccccc34)cc2)cc1]

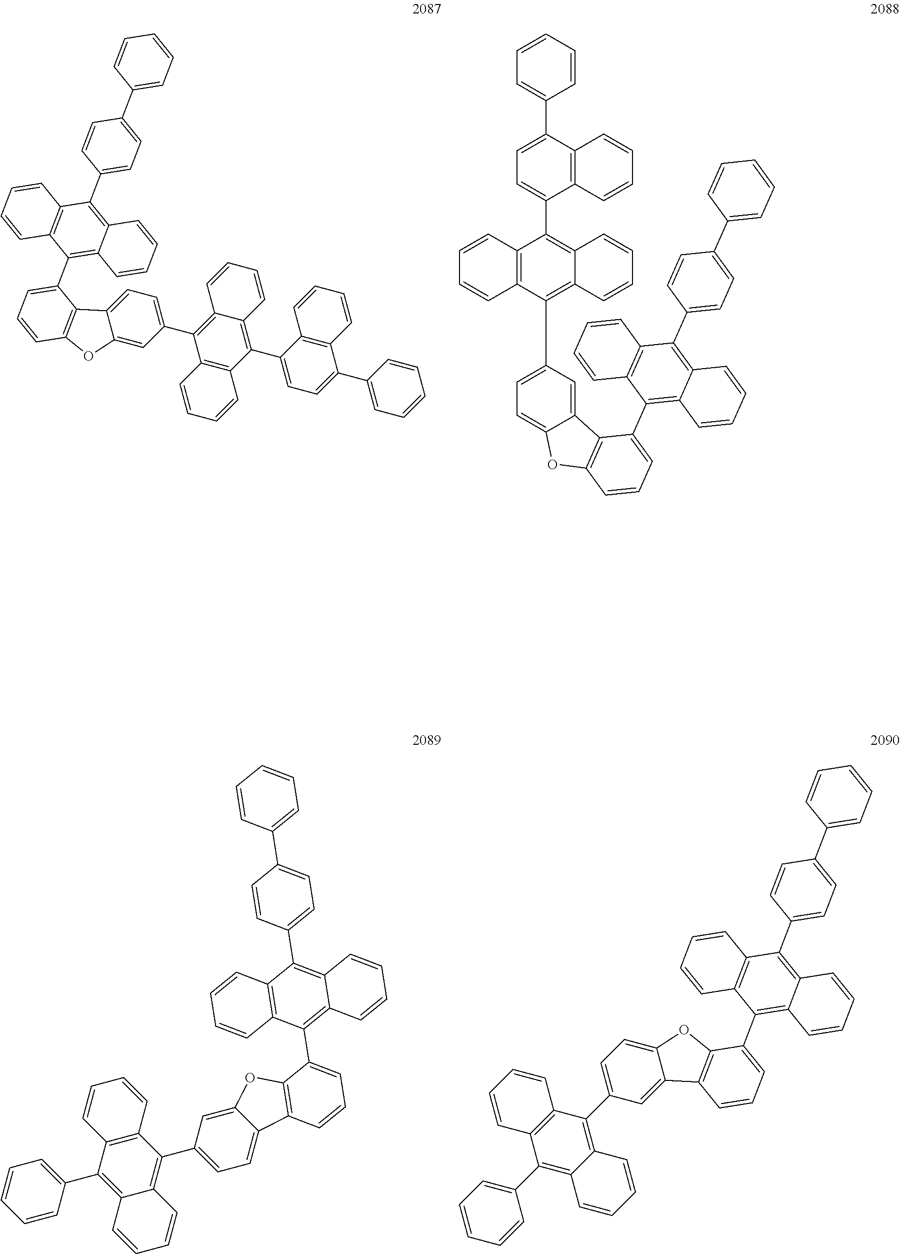 [SMILES: c1ccc(-c2ccc(-c3c4ccccc4c(-c4cccc5oc6cc(-c7c8ccccc8c(-c8ccc(-c9ccccc9)c9ccccc89)c8ccccc78)ccc6c45)c4ccccc34)cc2)cc1 ; c1ccc(-c2ccc(-c3c4ccccc4c(-c4cccc5oc6ccc(-c7c8ccccc8c(-c8ccc(-c9ccccc9)c9ccccc89)c8ccccc78)cc6c45)c4ccccc34)cc2)cc1 ; c1ccc(-c2ccc(-c3c4ccccc4c(-c4cccc5c4oc4cc(-c6c7ccccc7c(-c7ccccc7)c7ccccc67)ccc45)c4ccccc34)cc2)cc1 ; c1ccc(-c2ccc(-c3c4ccccc4c(-c4cccc5c4oc4ccc(-c6c7ccccc7c(-c7ccccc7)c7ccccc67)cc45)c4ccccc34)cc2)cc1]

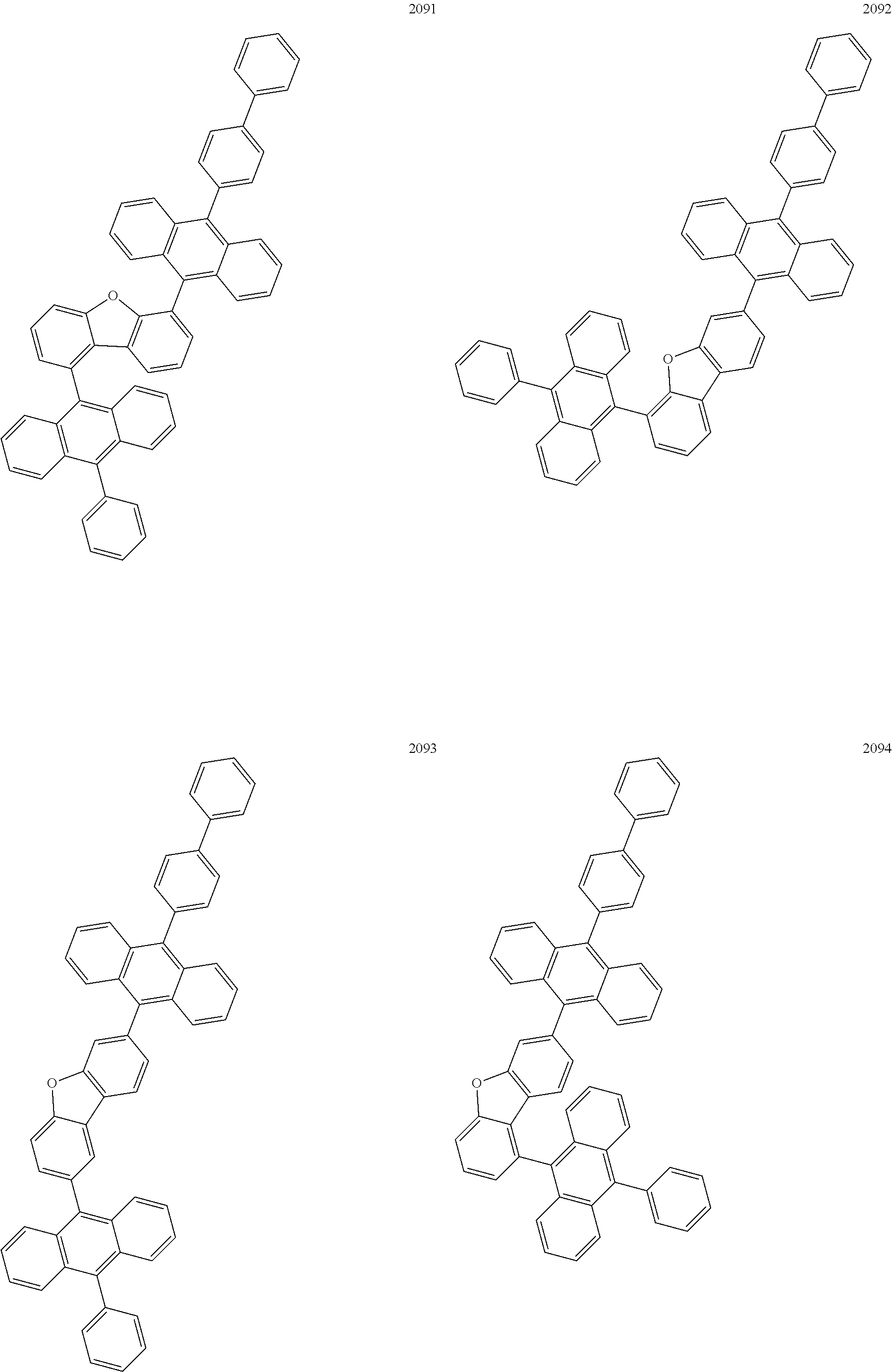 [SMILES: c1ccc(-c2ccc(-c3c4ccccc4c(-c4cccc5c4oc4cccc(-c6c7ccccc7c(-c7ccccc7)c7ccccc67)c45)c4ccccc34)cc2)cc1 ; c1ccc(-c2ccc(-c3c4ccccc4c(-c4ccc5c(c4)oc4c(-c6c7ccccc7c(-c7ccccc7)c7ccccc67)cccc45)c4ccccc34)cc2)cc1 ; c1ccc(-c2ccc(-c3c4ccccc4c(-c4ccc5c(c4)oc4ccc(-c6c7ccccc7c(-c7ccccc7)c7ccccc67)cc45)c4ccccc34)cc2)cc1 ; c1ccc(-c2ccc(-c3c4ccccc4c(-c4ccc5c(c4)oc4cccc(-c6c7ccccc7c(-c7ccccc7)c7ccccc67)c45)c4ccccc34)cc2)cc1]

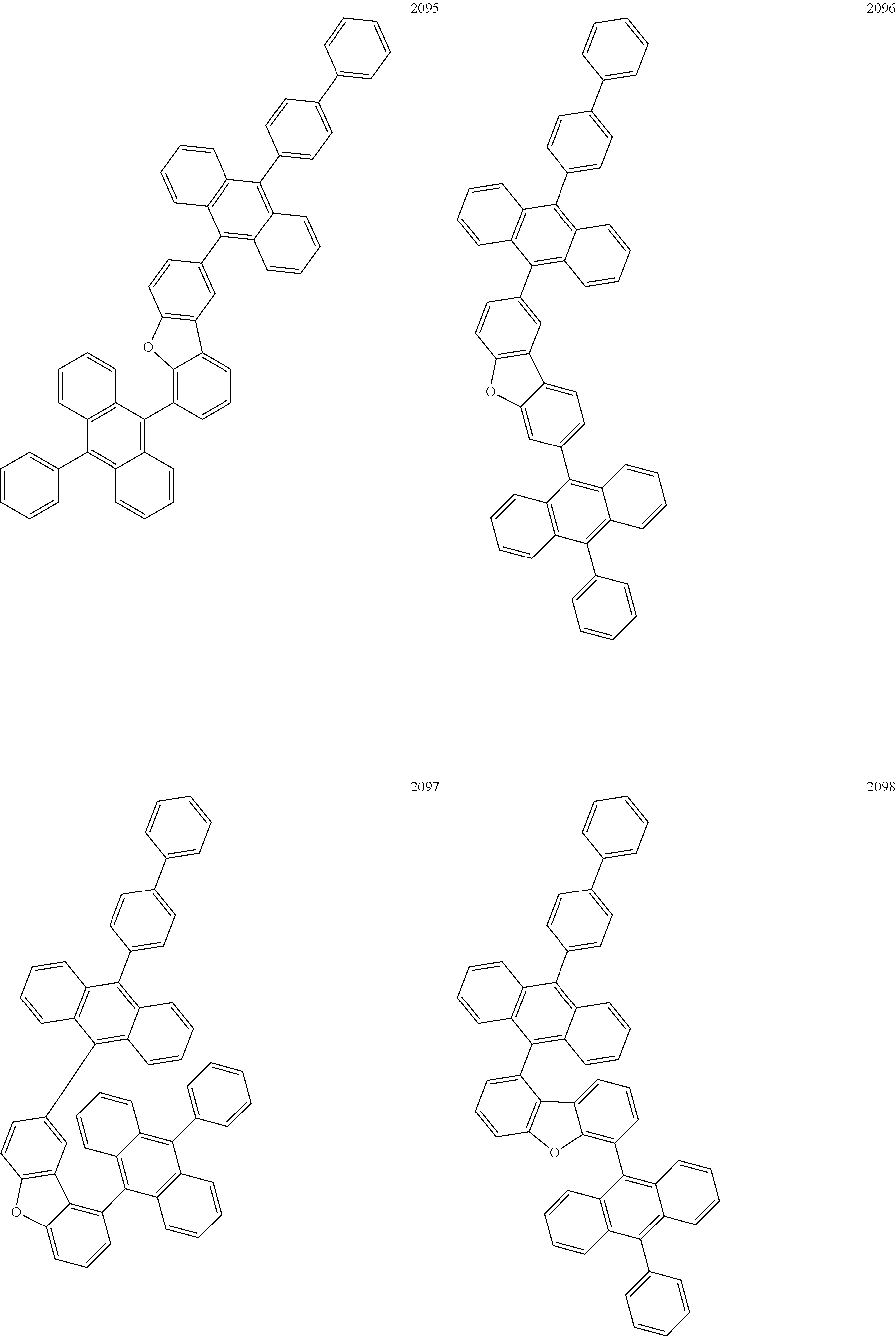 [SMILES: c1ccc(-c2ccc(-c3c4ccccc4c(-c4ccc5oc6c(-c7c8ccccc8c(-c8ccccc8)c8ccccc78)cccc6c5c4)c4ccccc34)cc2)cc1 ; c1ccc(-c2ccc(-c3c4ccccc4c(-c4ccc5oc6cc(-c7c8ccccc8c(-c8ccccc8)c8ccccc78)ccc6c5c4)c4ccccc34)cc2)cc1 ; c1ccc(-c2ccc(-c3c4ccccc4c(-c4ccc5oc6cccc(-c7c8ccccc8c(-c8ccccc8)c8ccccc78)c6c5c4)c4ccccc34)cc2)cc1 ; c1ccc(-c2ccc(-c3c4ccccc4c(-c4cccc5oc6c(-c7c8ccccc8c(-c8ccccc8)c8ccccc78)cccc6c45)c4ccccc34)cc2)cc1]

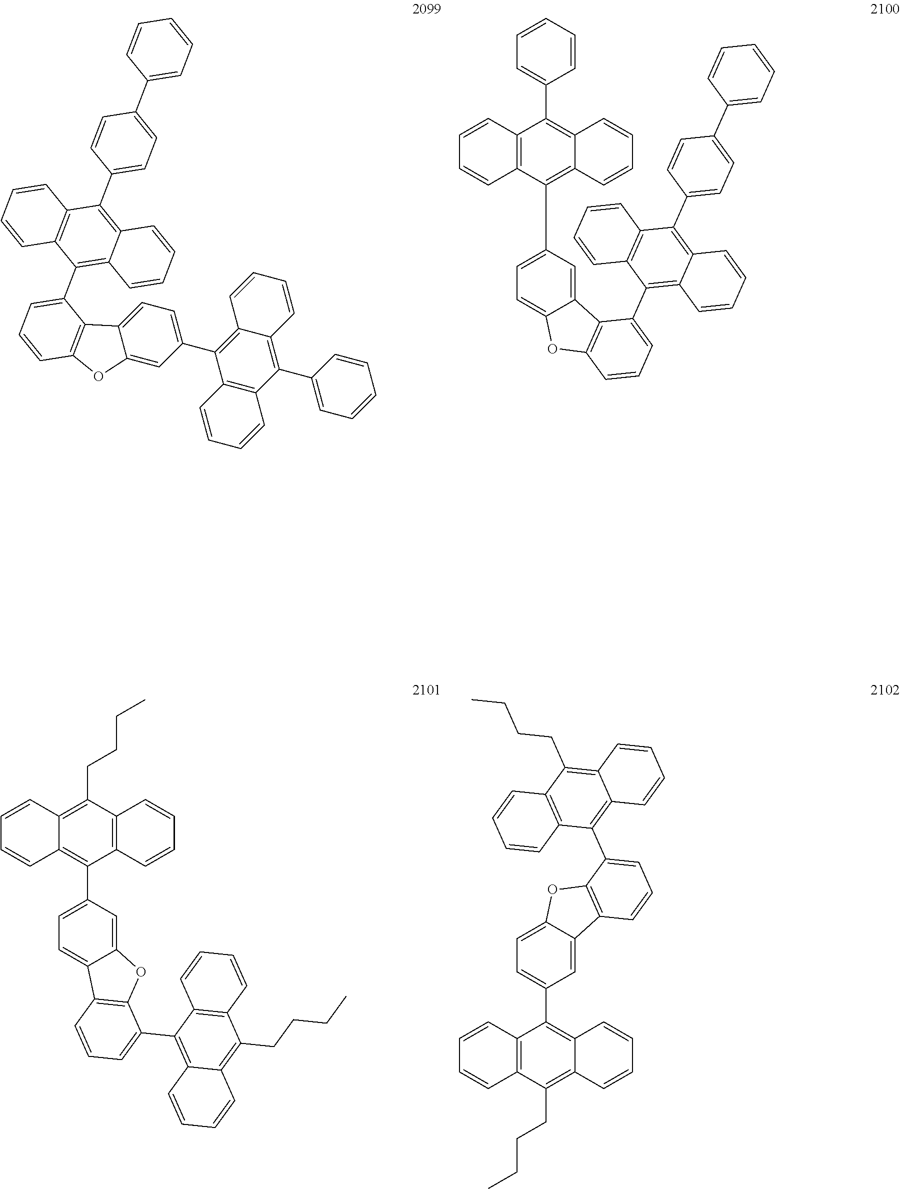 [SMILES: c1ccc(-c2ccc(-c3c4ccccc4c(-c4cccc5oc6cc(-c7c8ccccc8c(-c8ccccc8)c8ccccc78)ccc6c45)c4ccccc34)cc2)cc1 ; c1ccc(-c2ccc(-c3c4ccccc4c(-c4cccc5oc6ccc(-c7c8ccccc8c(-c8ccccc8)c8ccccc78)cc6c45)c4ccccc34)cc2)cc1 ; CCCCc1c2ccccc2c(-c2ccc3c(c2)oc2c(-c4c5ccccc5c(CCCC)c5ccccc45)cccc23)c2ccccc12 ; CCCCc1c2ccccc2c(-c2ccc3oc4c(-c5c6ccccc6c(CCCC)c6ccccc56)cccc4c3c2)c2ccccc12]

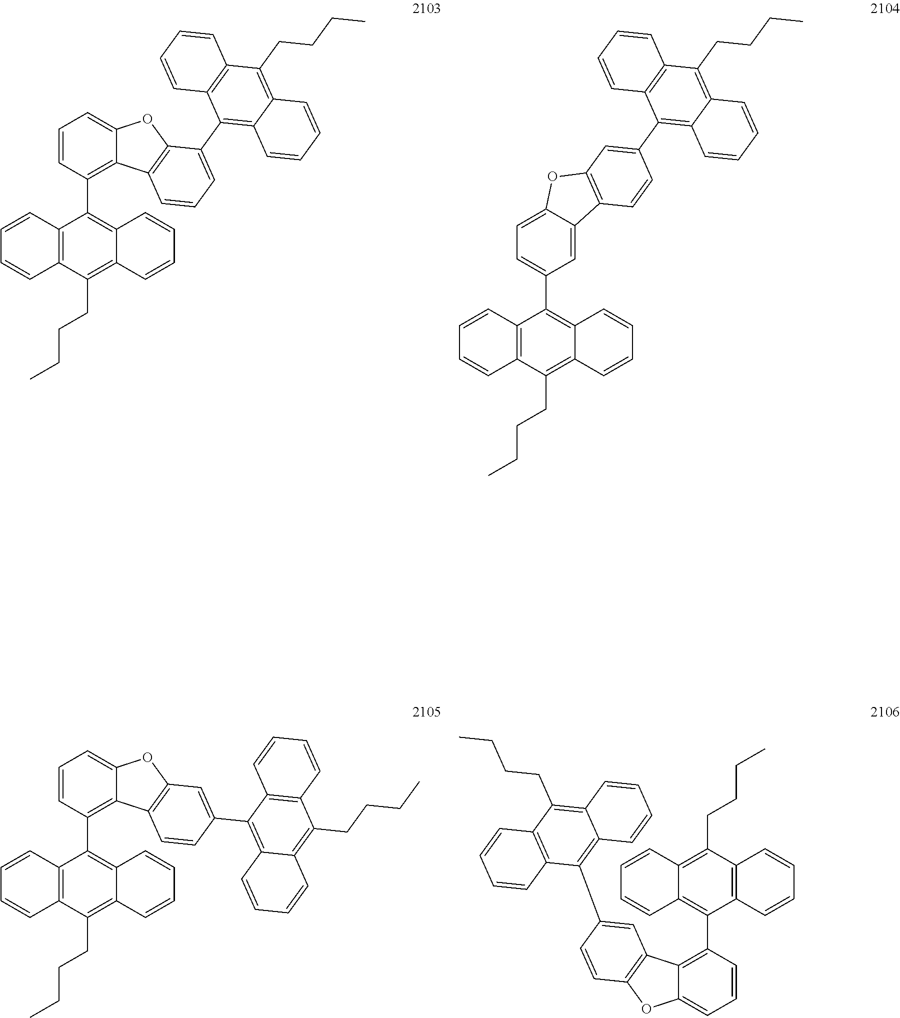 [SMILES: CCCCc1c2ccccc2c(-c2cccc3c2oc2cccc(-c4c5ccccc5c(CCCC)c5ccccc45)c23)c2ccccc12 ; CCCCc1c2ccccc2c(-c2ccc3c(c2)oc2ccc(-c4c5ccccc5c(CCCC)c5ccccc45)cc23)c2ccccc12 ; CCCCc1c2ccccc2c(-c2ccc3c(c2)oc2cccc(-c4c5ccccc5c(CCCC)c5ccccc45)c23)c2ccccc12 ; CCCCc1c2ccccc2c(-c2ccc3oc4cccc(-c5c6ccccc6c(CCCC)c6ccccc56)c4c3c2)c2ccccc12]

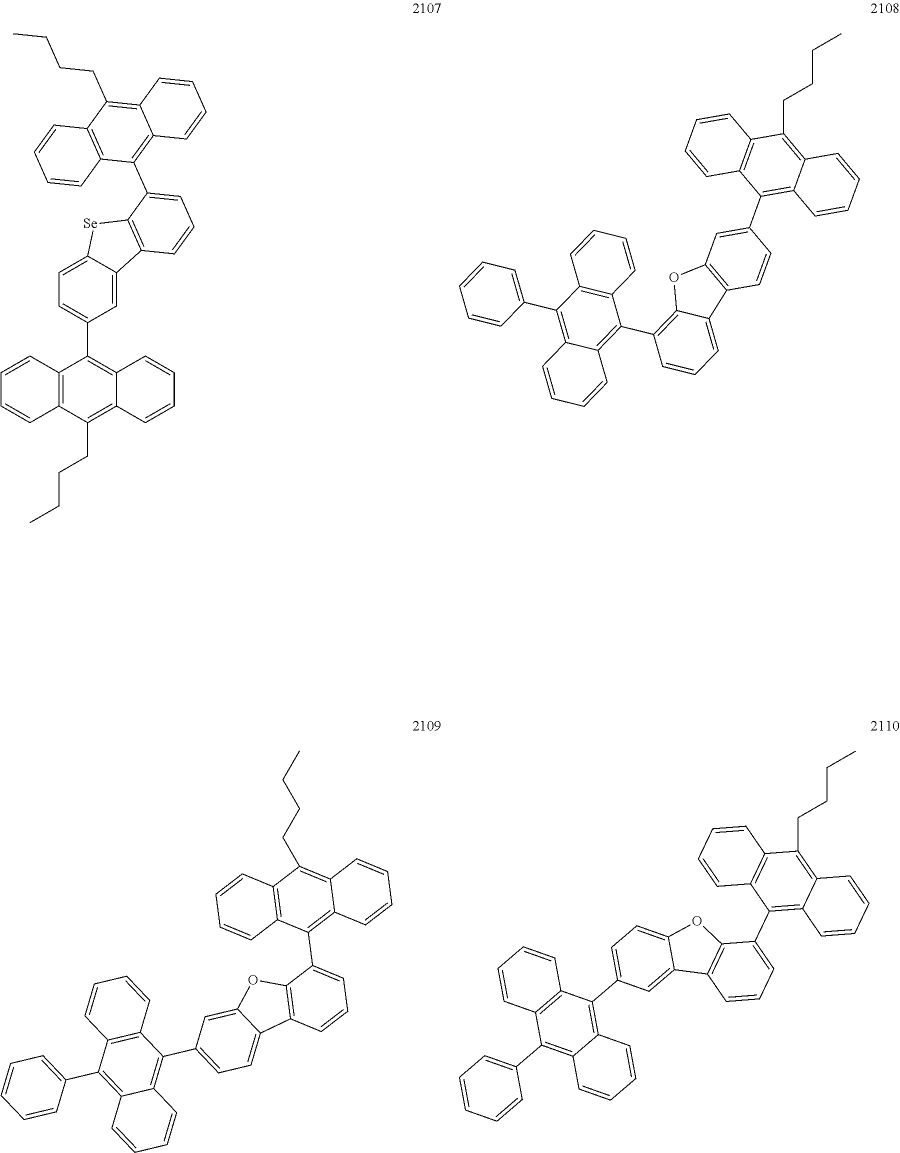 [SMILES: CCCCc1c2ccccc2c(-c2ccc3[se]c4c(-c5c6ccccc6c(CCCC)c6ccccc56)cccc4c3c2)c2ccccc12 ; CCCCc1c2ccccc2c(-c2ccc3c(c2)oc2c(-c4c5ccccc5c(-c5ccccc5)c5ccccc45)cccc23)c2ccccc12 ; CCCCc1c2ccccc2c(-c2cccc3c2oc2cc(-c4c5ccccc5c(-c5ccccc5)c5ccccc45)ccc23)c2ccccc12 ; CCCCc1c2ccccc2c(-c2cccc3c2oc2ccc(-c4c5ccccc5c(-c5ccccc5)c5ccccc45)cc23)c2ccccc12]

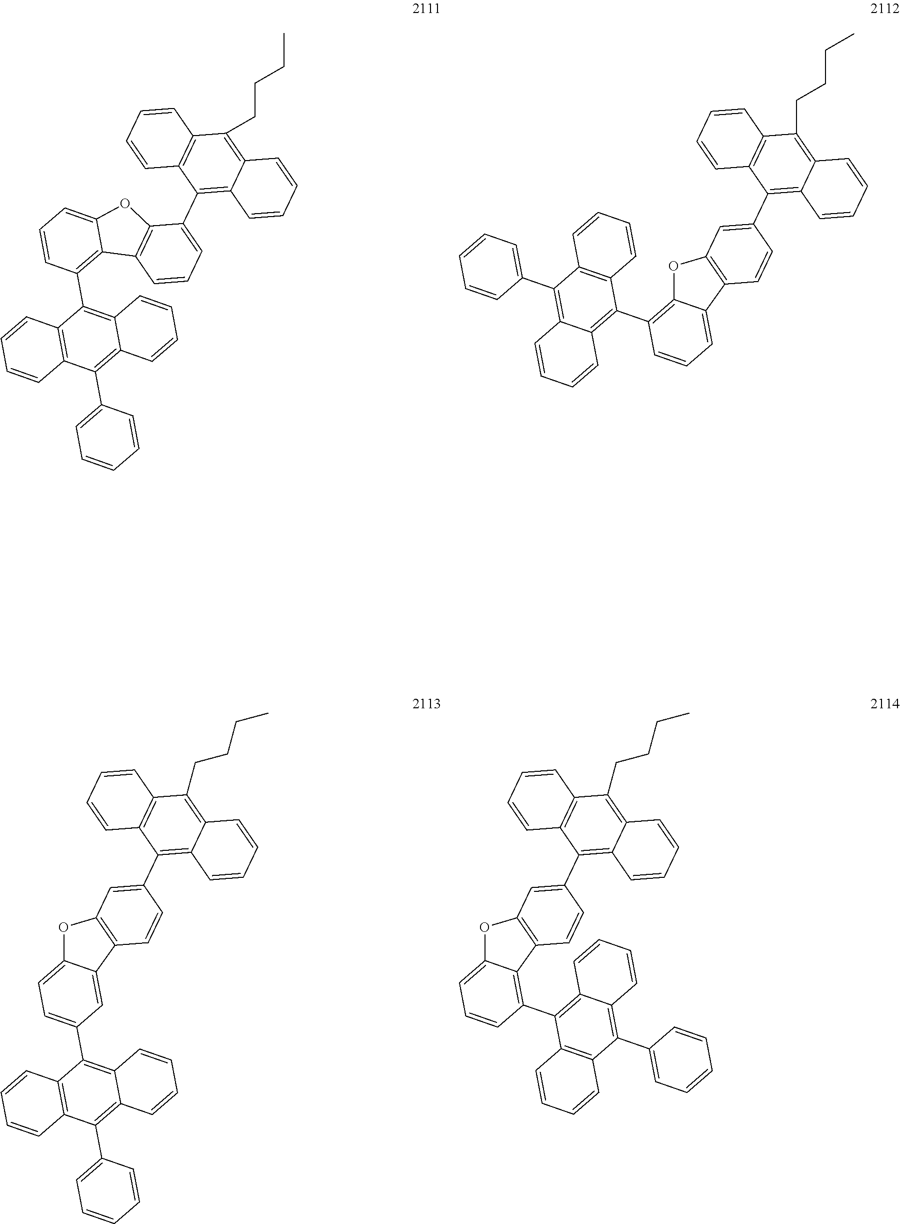 [SMILES: CCCCc1c2ccccc2c(-c2cccc3c2oc2cccc(-c4c5ccccc5c(-c5ccccc5)c5ccccc45)c23)c2ccccc12 ; CCCCc1c2ccccc2c(-c2ccc3c(c2)oc2c(-c4c5ccccc5c(-c5ccccc5)c5ccccc45)cccc23)c2ccccc12 ; CCCCc1c2ccccc2c(-c2ccc3c(c2)oc2ccc(-c4c5ccccc5c(-c5ccccc5)c5ccccc45)cc23)c2ccccc12 ; CCCCc1c2ccccc2c(-c2ccc3c(c2)oc2cccc(-c4c5ccccc5c(-c5ccccc5)c5ccccc45)c23)c2ccccc12]

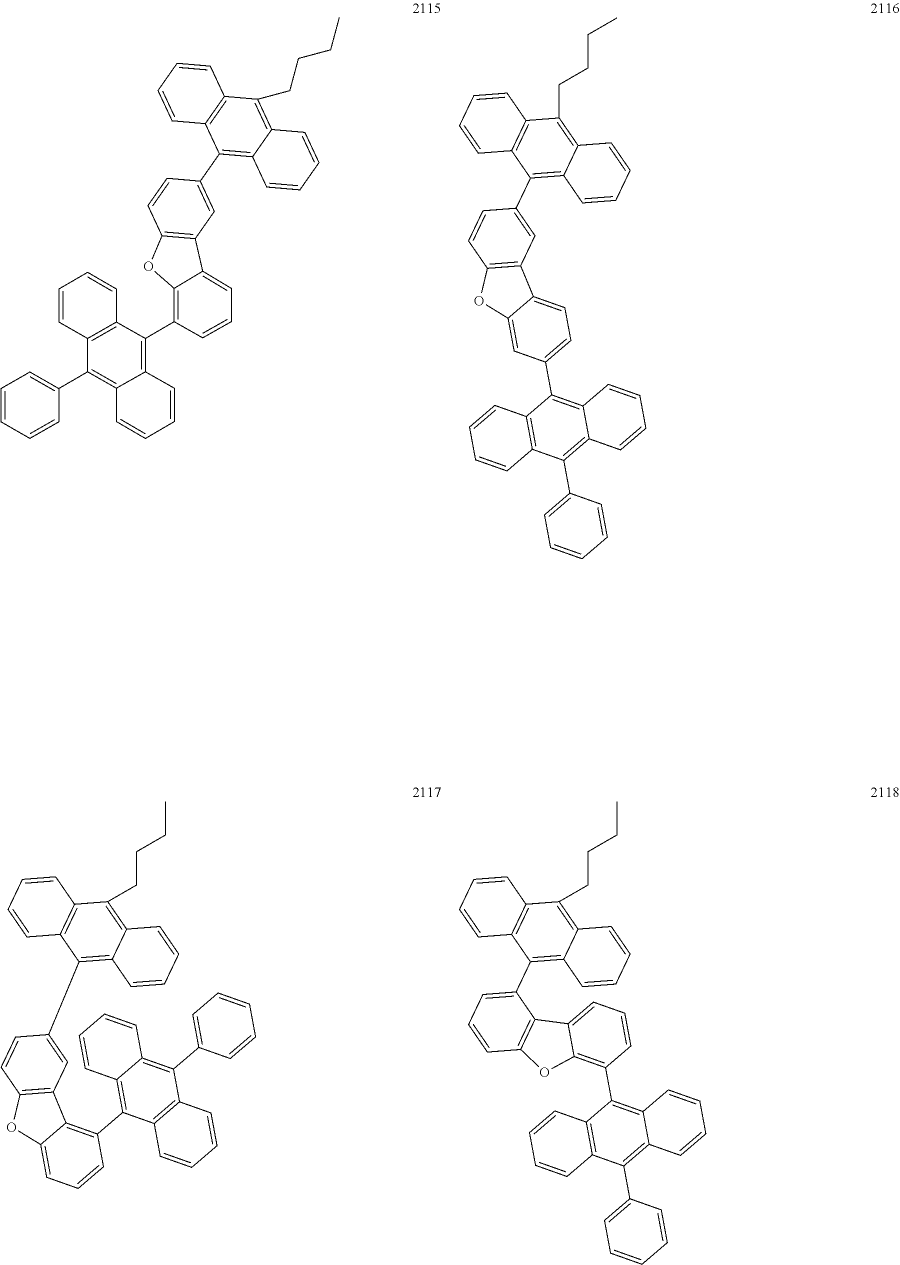 [SMILES: CCCCc1c2ccccc2c(-c2ccc3oc4c(-c5c6ccccc6c(-c6ccccc6)c6ccccc56)cccc4c3c2)c2ccccc12 ; CCCCc1c2ccccc2c(-c2ccc3oc4cc(-c5c6ccccc6c(-c6ccccc6)c6ccccc56)ccc4c3c2)c2ccccc12 ; CCCCc1c2ccccc2c(-c2ccc3oc4cccc(-c5c6ccccc6c(-c6ccccc6)c6ccccc56)c4c3c2)c2ccccc12 ; CCCCc1c2ccccc2c(-c2cccc3oc4c(-c5c6ccccc6c(-c6ccccc6)c6ccccc56)cccc4c23)c2ccccc12]

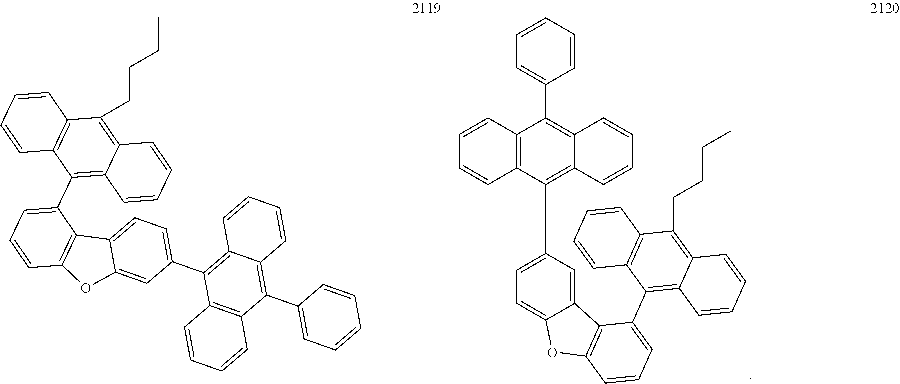 [SMILES: CCCCc1c2ccccc2c(-c2cccc3oc4cc(-c5c6ccccc6c(-c6ccccc6)c6ccccc56)ccc4c23)c2ccccc12 ; CCCCc1c2ccccc2c(-c2cccc3oc4ccc(-c5c6ccccc6c(-c6ccccc6)c6ccccc56)cc4c23)c2ccccc12]

When the host is a mixture of an electron-transporting host and a hole-transporting host, the weight ratio of the electron-transporting host and the hole-transporting host may be about 1:9 to 9:1, for example, about 2:8 to 8:2, for example, about 4:6 to 6:4, for example, about 5:5. When the weight ratio of the electron-transporting host and the hole-transporting host satisfies the above-described ranges, the hole-and-electron-transporting balance in the emission layer 15 may be made.

Dopant

The dopant includes the condensed cyclic compound.

Sensitizer

In an embodiment, the sensitizer may be a phosphorescent sensitizer including at least one metal Period 1 transition metal, Period 2 transition metal, Period 3 transition metal, or any combination thereof.

In an embodiment, the sensitizer may include metal (M₁₁) of at least one a first-row transition metal of the Periodic Table of Elements, a second-row transition metal of the Periodic Table of Elements, a third-row transition metal of the Periodic Table of Elements, or any combination thereof, and an organic ligand (L₁), and L₁₁ and M₁₁ may form 1, 2, 3, or 4 cyclometallated rings.

In an embodiment, the sensitizer may include an organometallic compound represented by Formula 101:

M₁₁(L₁₁)ₙ₁₁(L₁₂)ₙ₁₂ Formula 101

wherein, in Formula 101,

M₁₁ may be a first-row transition metal of the Periodic Table of Elements, a second-row transition metal of the Periodic Table of Elements, or a third-row transition metal of the Periodic Table of Elements;

L₁₁ is a ligand represented by one of Formulae 1-1 to 1-4;

L₁₂ is a monodentate ligand or a bidentate ligand;

n11 is 1,

n12 is 0, 1, or 2;

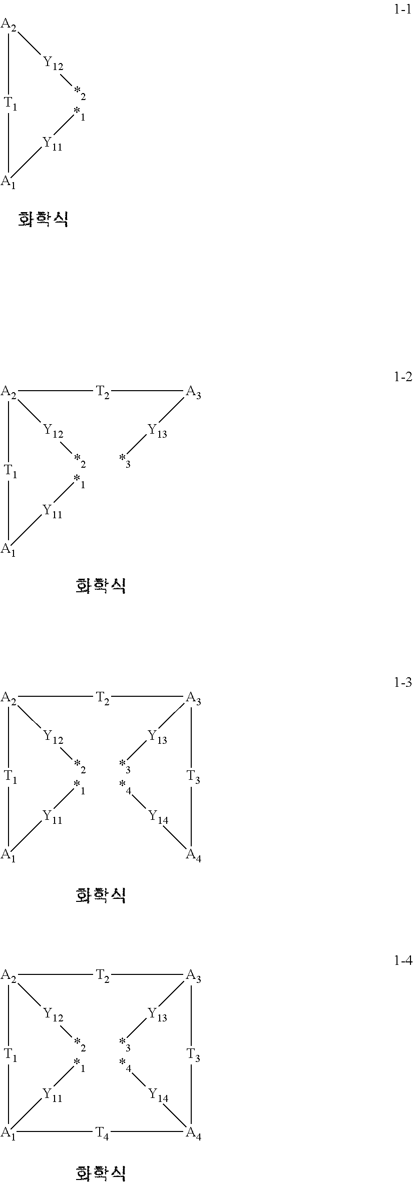

wherein, in Formulae 1-1 to 1-4,

A₁ to A₄ may each independently be a substituted or unsubstituted C₅-C₃₀ carbocyclic group, a substituted or unsubstituted C₁-C₃₀ heterocyclic group, or a non-cyclic group,

Y₁₁ to Y₁₄ may each independently be a chemical bond, O, S, N(R₉₁), B(R₉₁), P(R₉₁), or C(R₉₁)(R₉₂),

T₁ to T₄ may each independently be a single bond, a double bond, *—N(R₉₃)—*′, *—B(R₉₃)—*′, *—P(R₉₃)—*′, *—C(R₉₃)(R₉₄)—*′, *—Si(R₉₃)(R₉₄)—*′, *—Ge(R₉₃)(R₉₄)—*′, *—S—*′, *—Se—*′, *—O—*′, *—C(═O)—*′, *—S(═O)—*′, *—S(═O)₂—*, *—C(R₉₃)═*′, *═C(R₉₃)—*′, *—C(R₉₃)═C(R₉₄)—*, *—C(═S)—*′, or *—C≡C—*′, and

a substituent of the substituted C₅-C₃₀ carbocyclic group, a substituent of the substituted C₁-C₃₀ heterocyclic group, and R₉₁ to R₉₄ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic heterocondensed polycyclic group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein a substituent of the substituted C₅-C₃₀ carbocyclic group and a substituent of the substituted C₁-C₃₀ heterocyclic group may not be hydrogen.

*₁, *₂, *₃, and *₄ each indicate a binding site to M₁₁, and

Q₁ to Q₃ may each independently be: hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a C₁-C₆₀ heteroaryloxy group, a C₁-C₆₀ heteroarylthio group, a monovalent aromatic condensed polycyclic group, a monovalent aromatic heterocondensed polycyclic group, a monovalent non-aromatic condensed polycyclic group, a monovalent non-aromatic condensed heteropolycyclic group; a C₁-C₆₀ alkyl group which is substituted with at least one deuterium, —F, cyano group, a C₁-C₆₀ alkyl group, a C₆-C₆₀ aryl group, or a combination thereof; or a C₆-C₆₀ aryl group which is substituted with at least one deuterium, —F, cyano group, a C₁-C₆₀ alkyl group, a C₆-C₆₀ aryl group, or a combination thereof.

In one or more embodiments, the sensitizer may be a compound of Groups I to VIII, but embodiments of the present disclosure are not limited thereto:

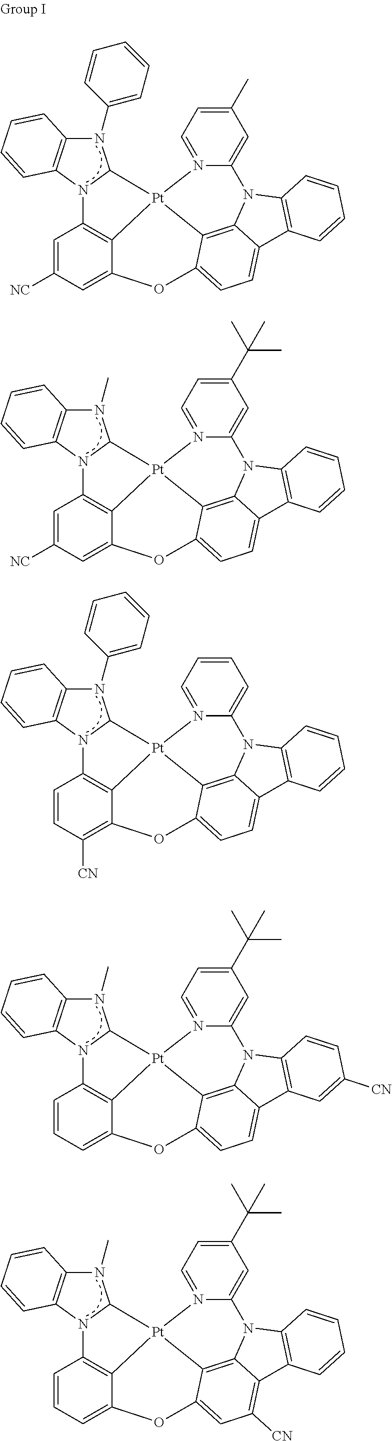

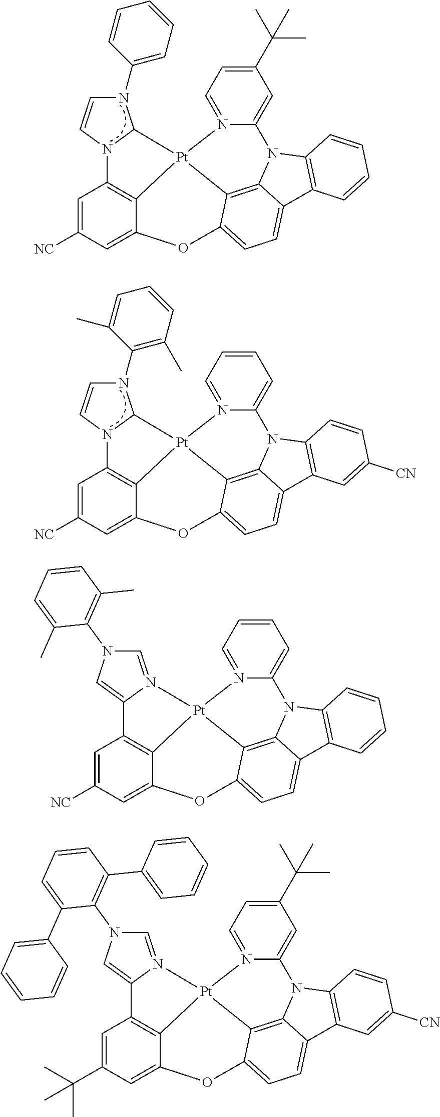

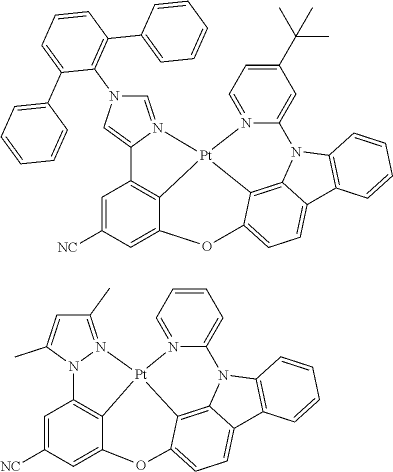

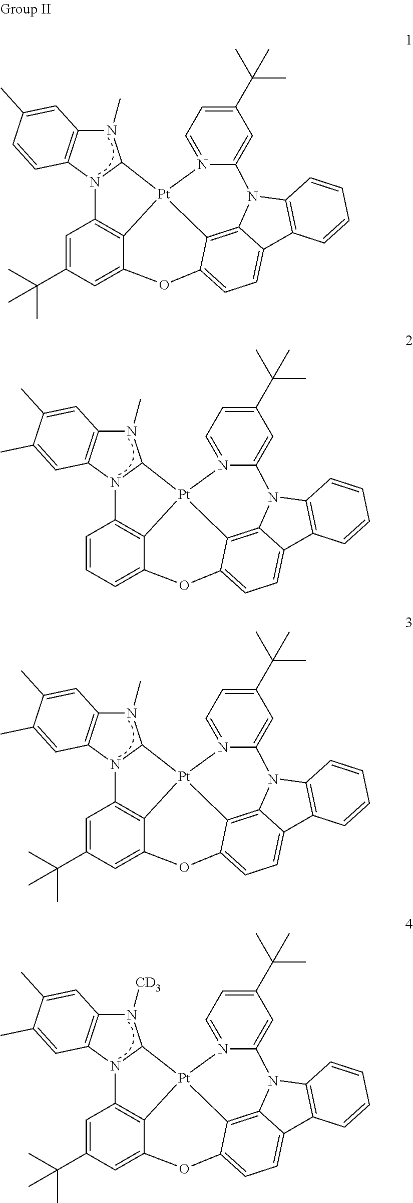

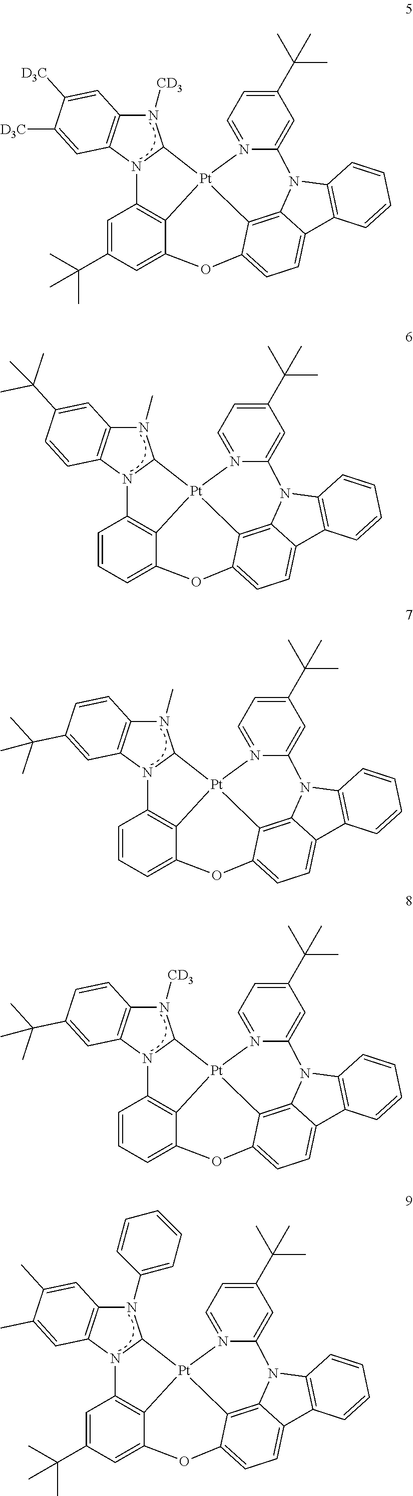

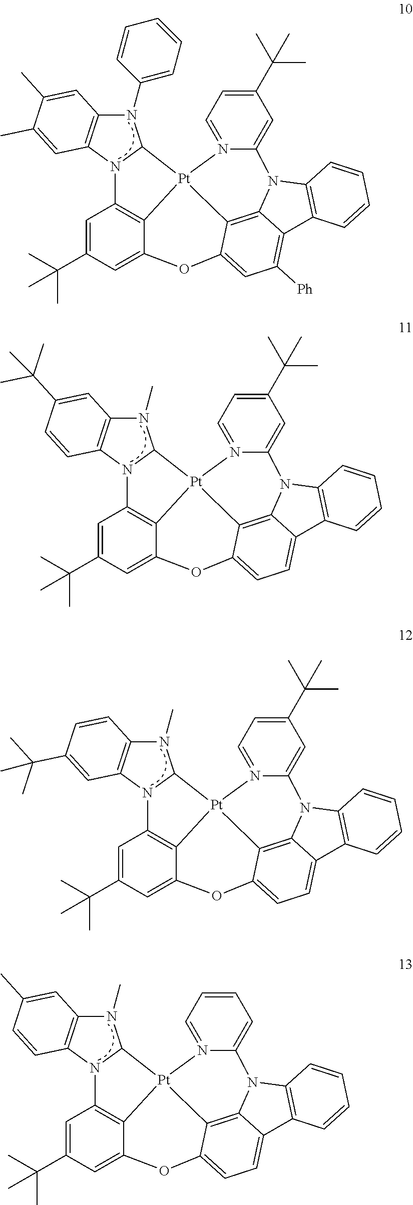

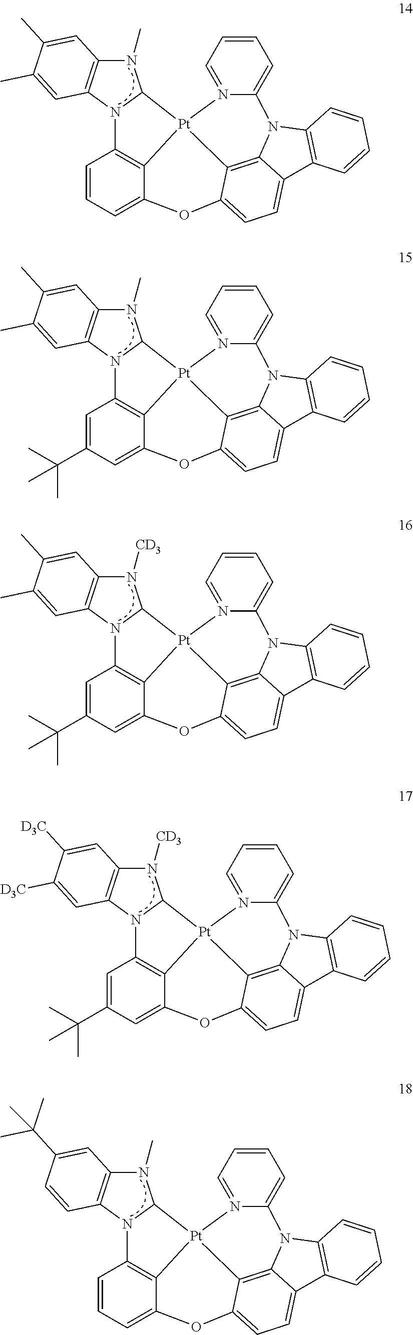

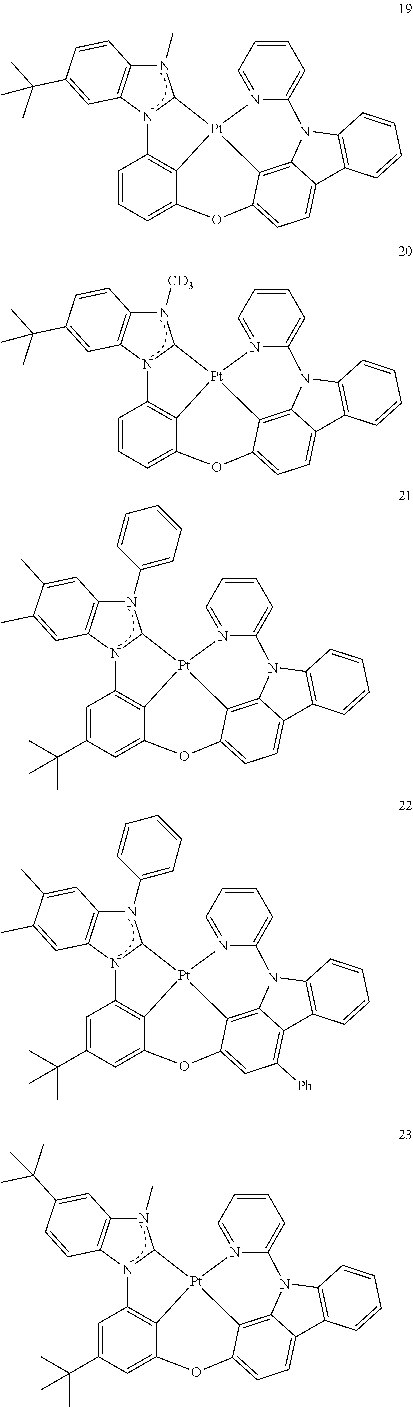

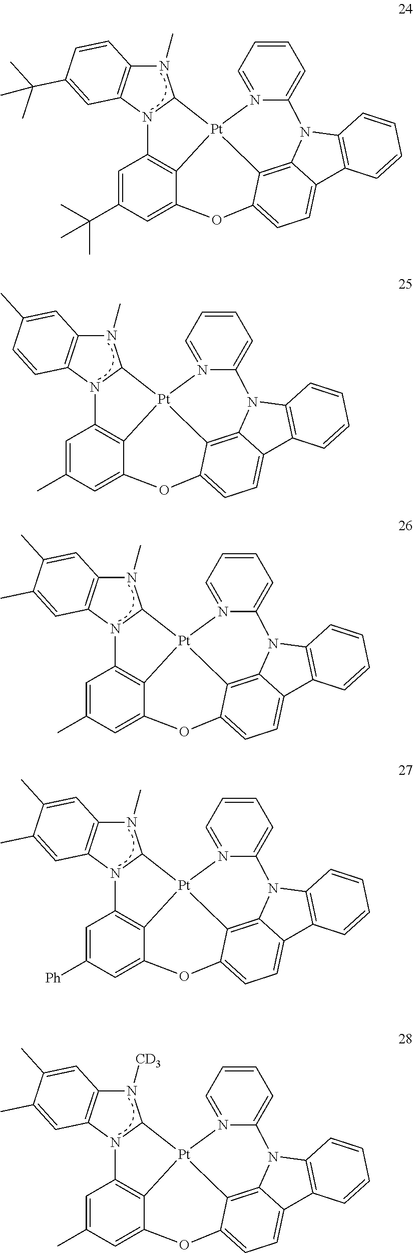

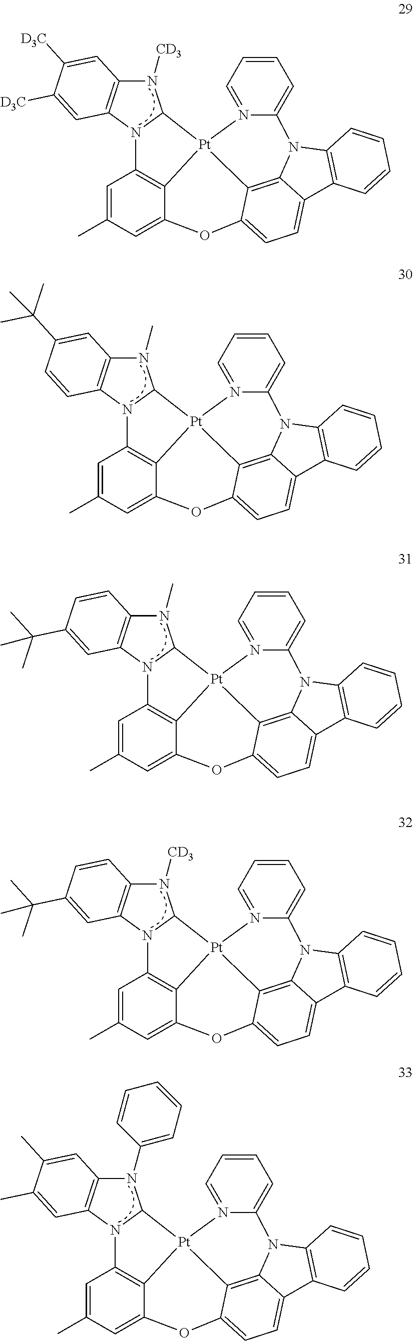

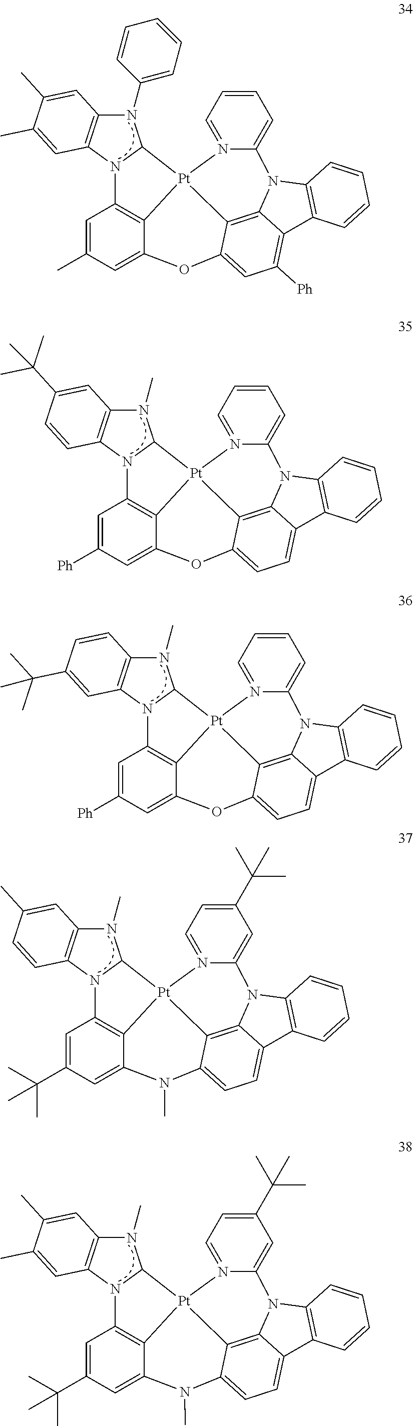

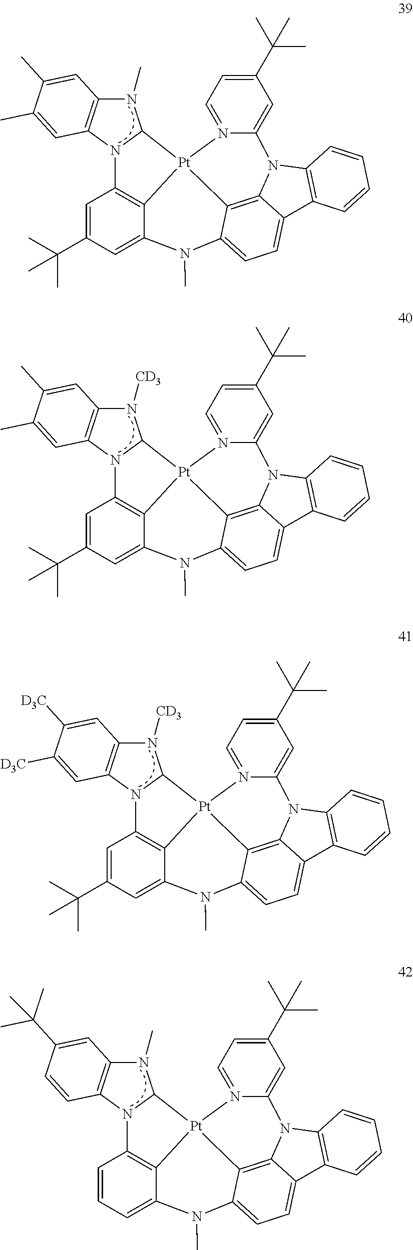

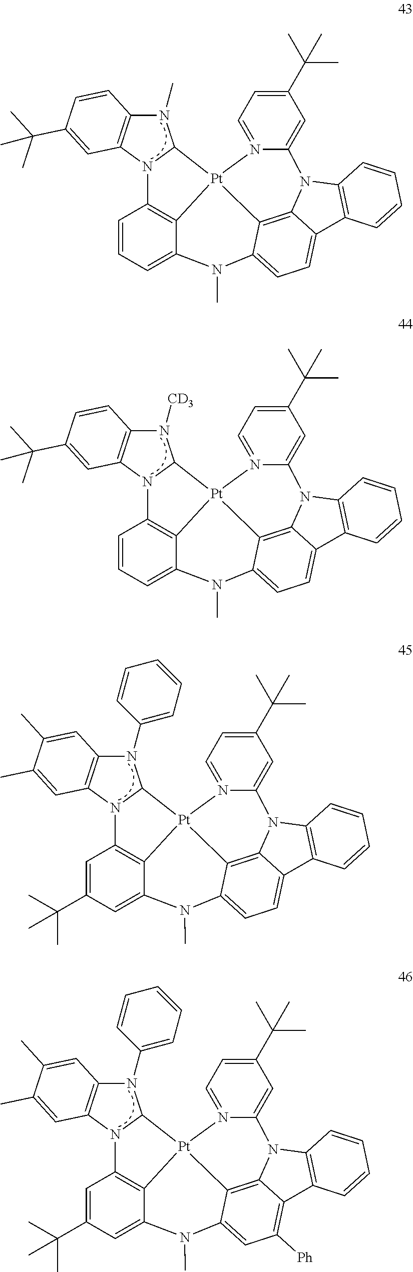

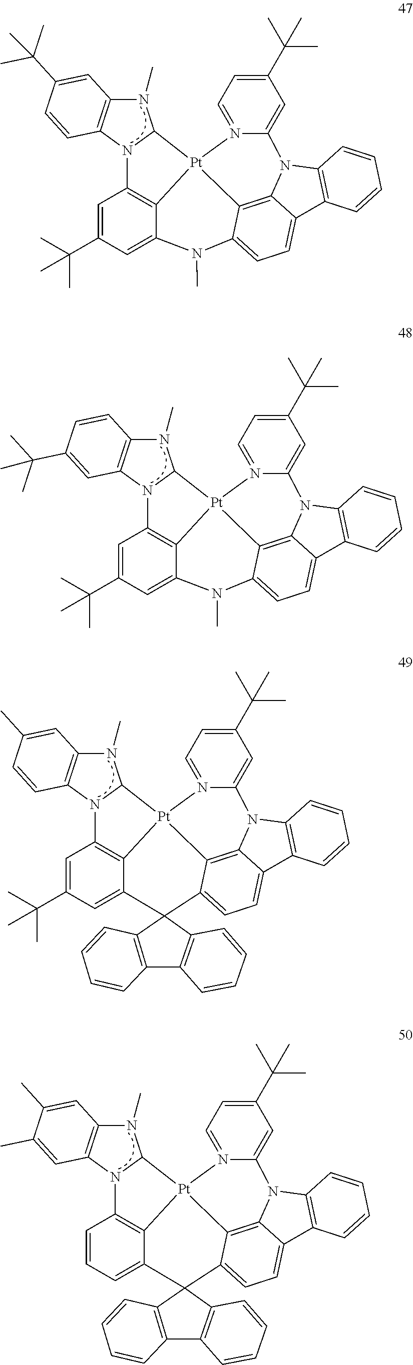

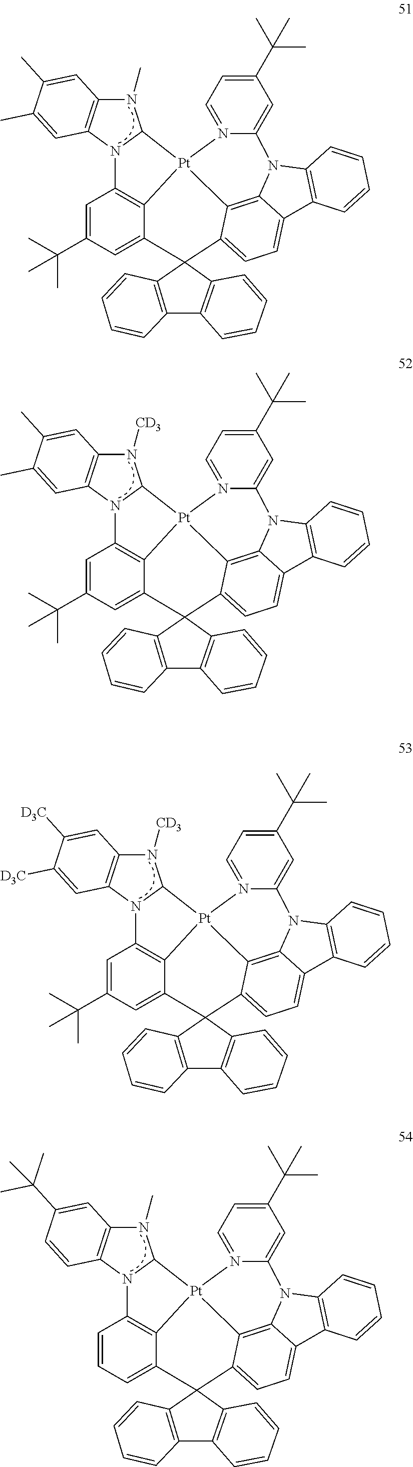

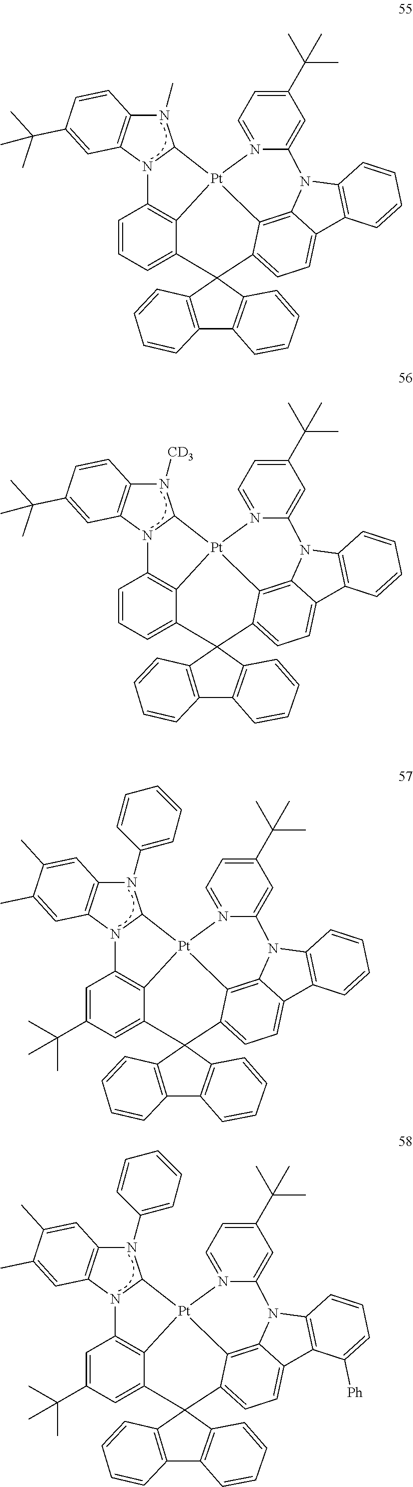

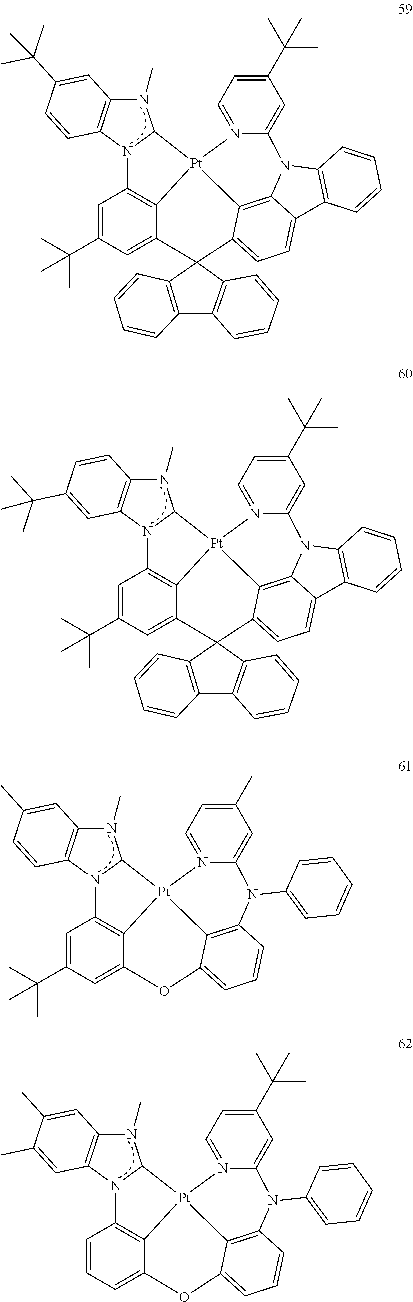

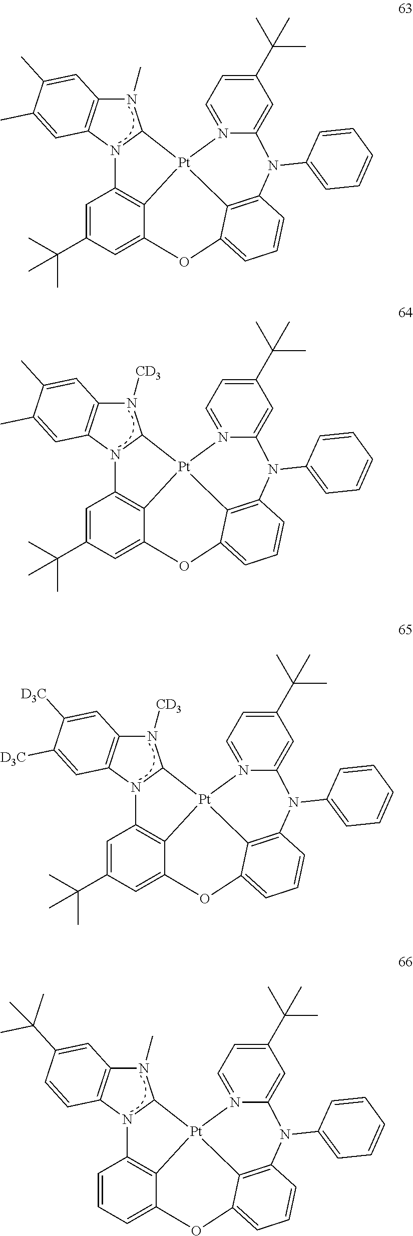

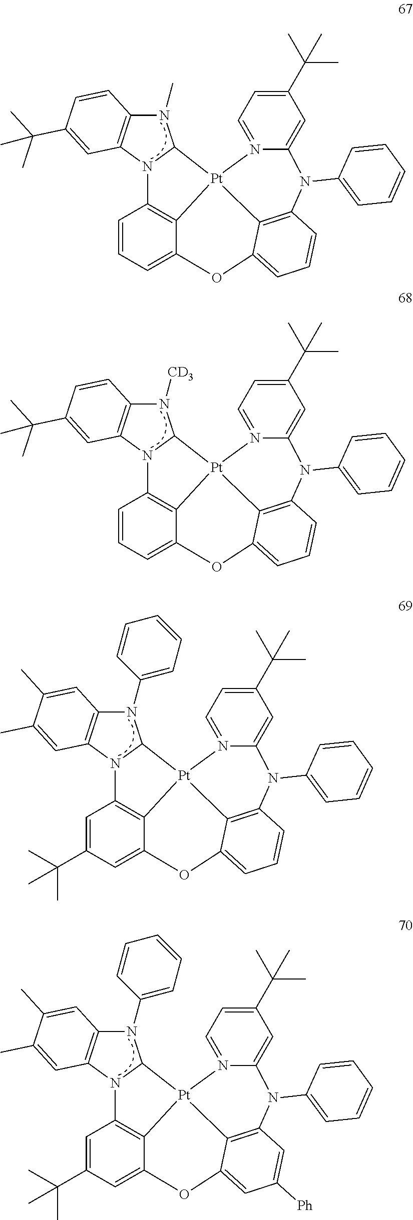

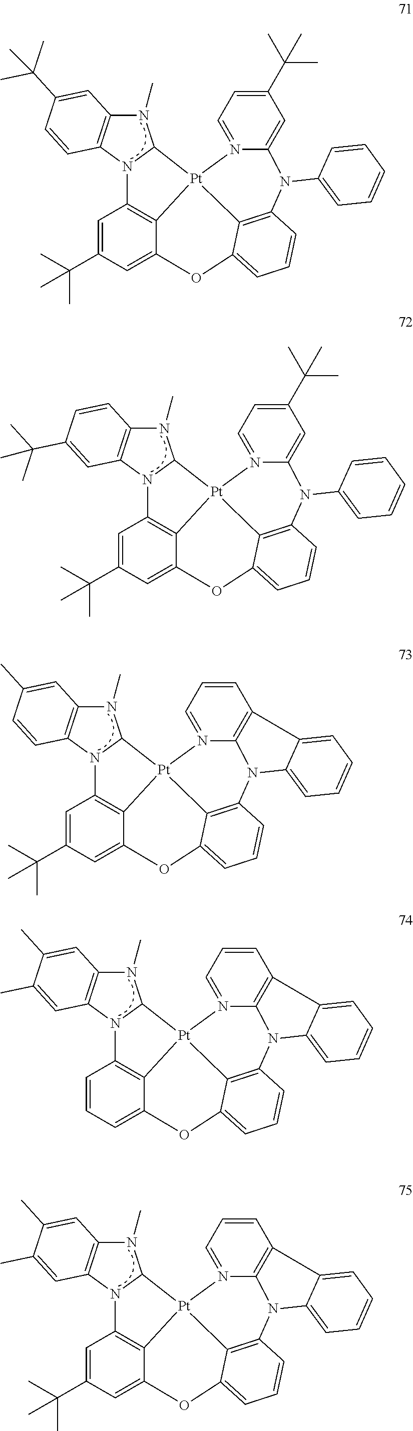

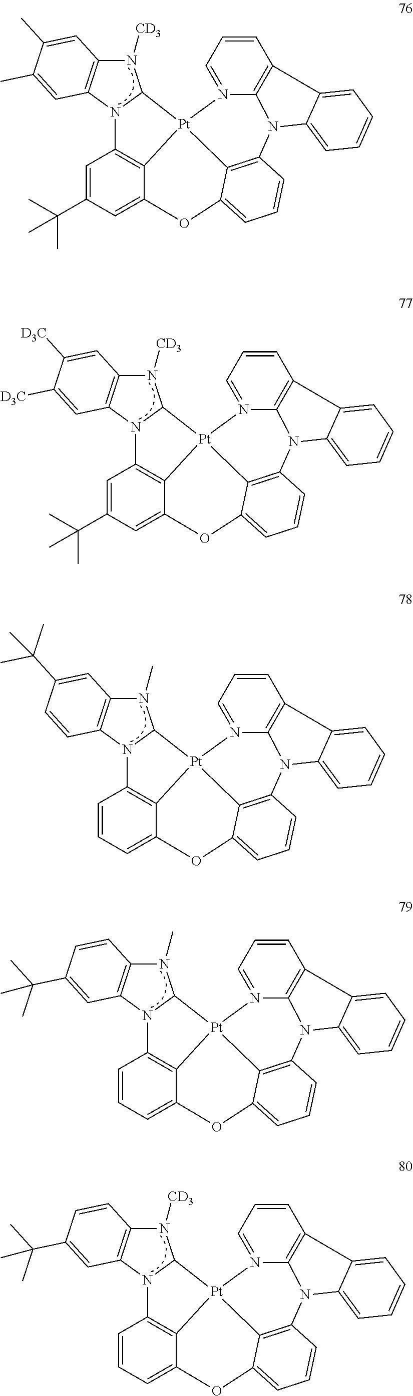

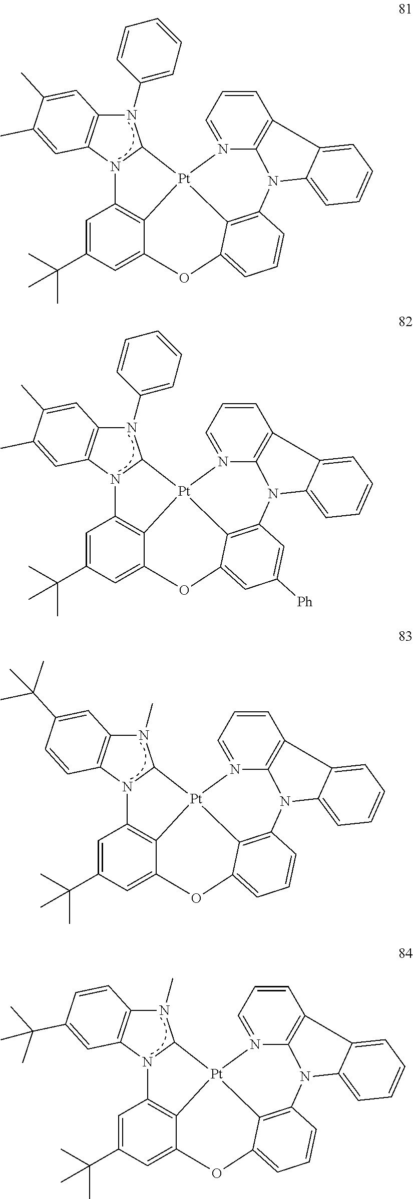

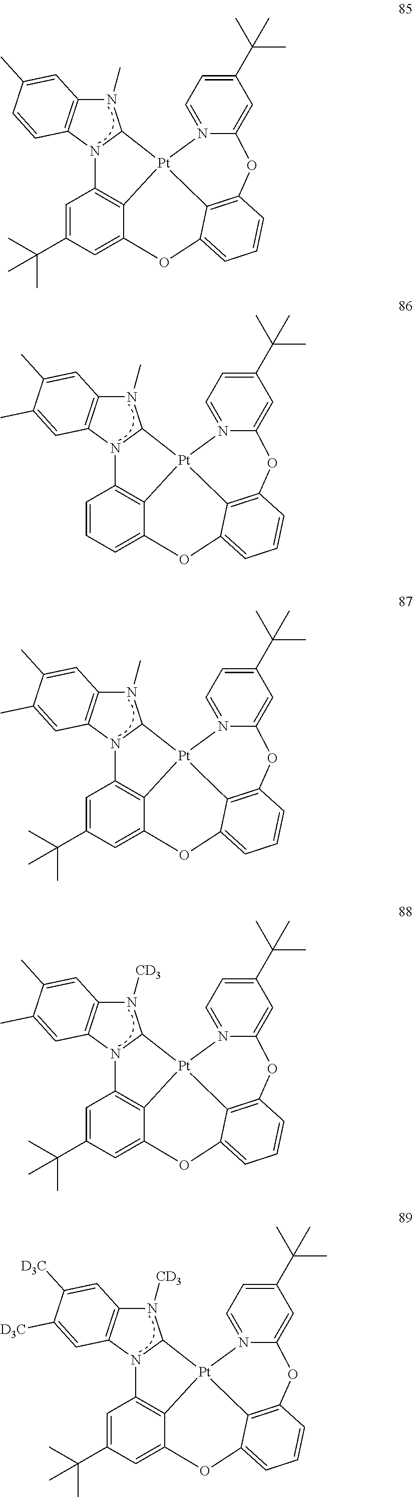

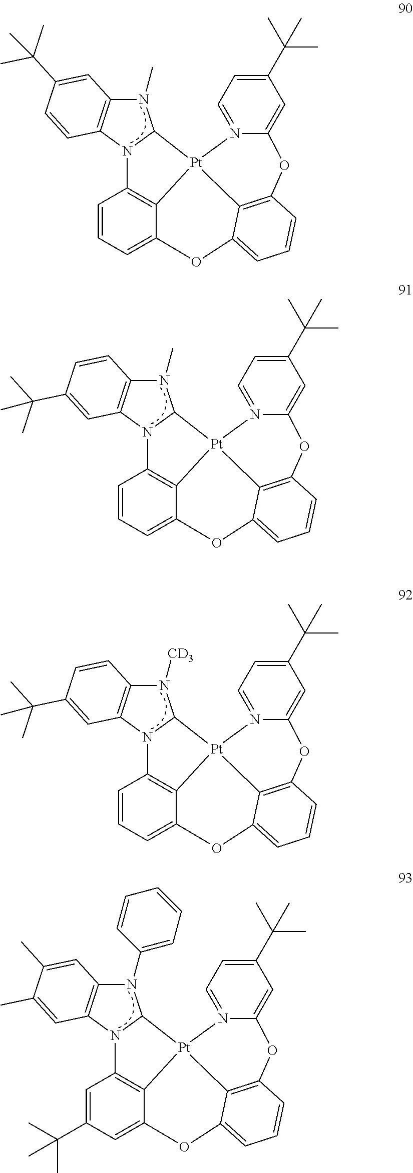

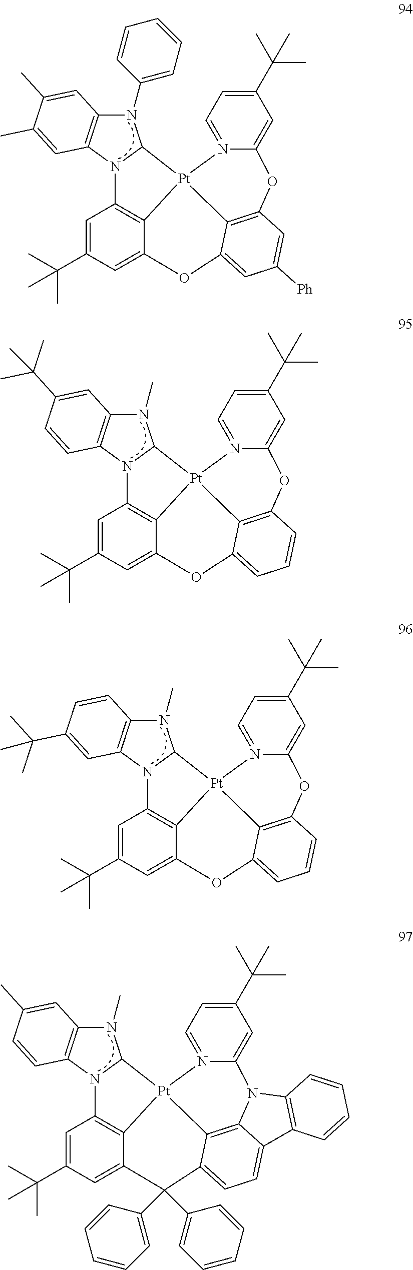

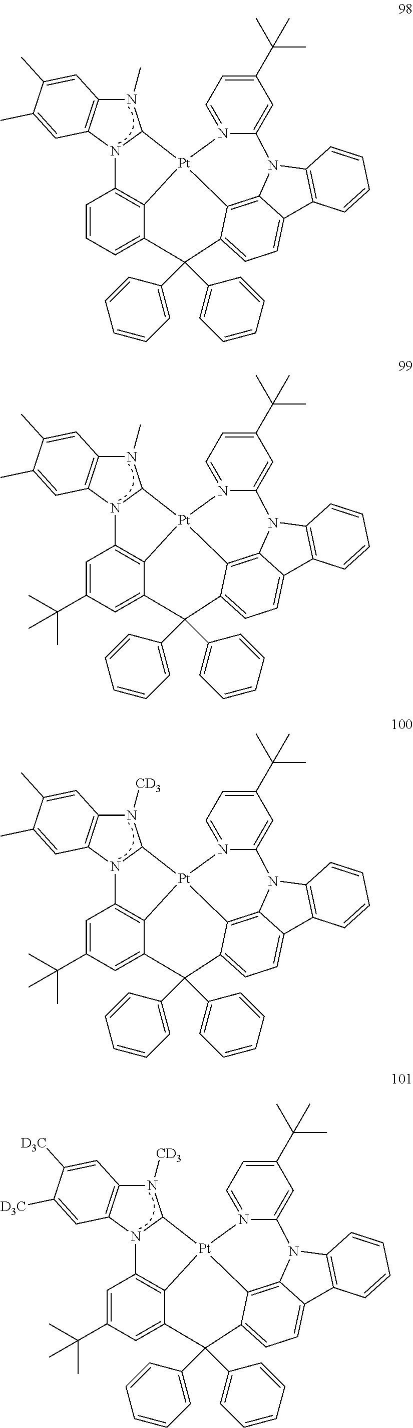

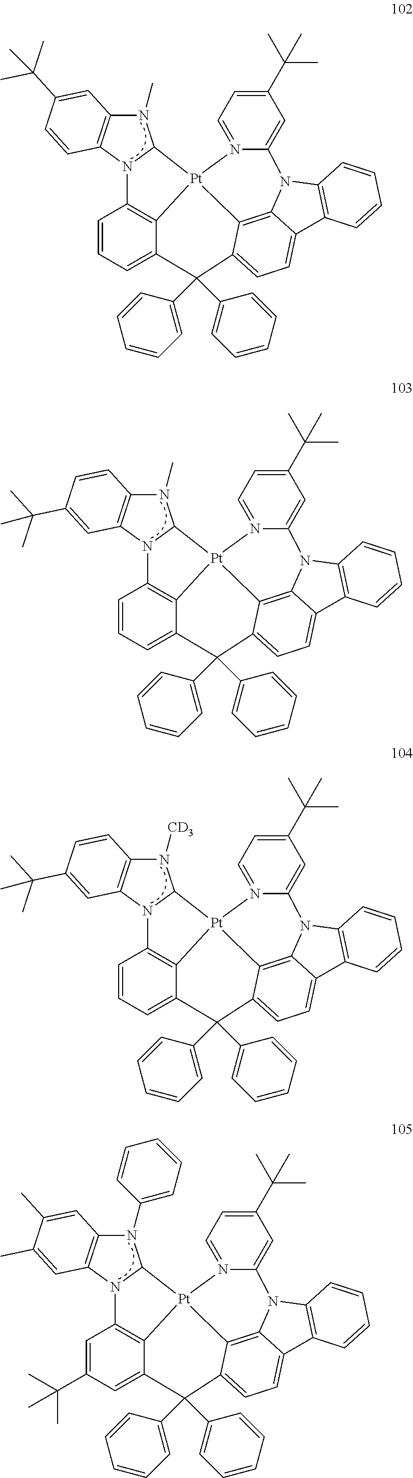

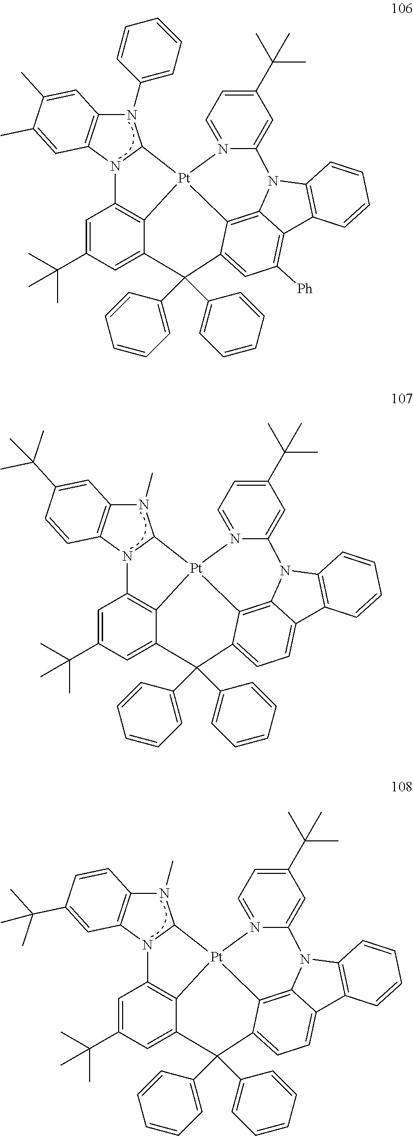

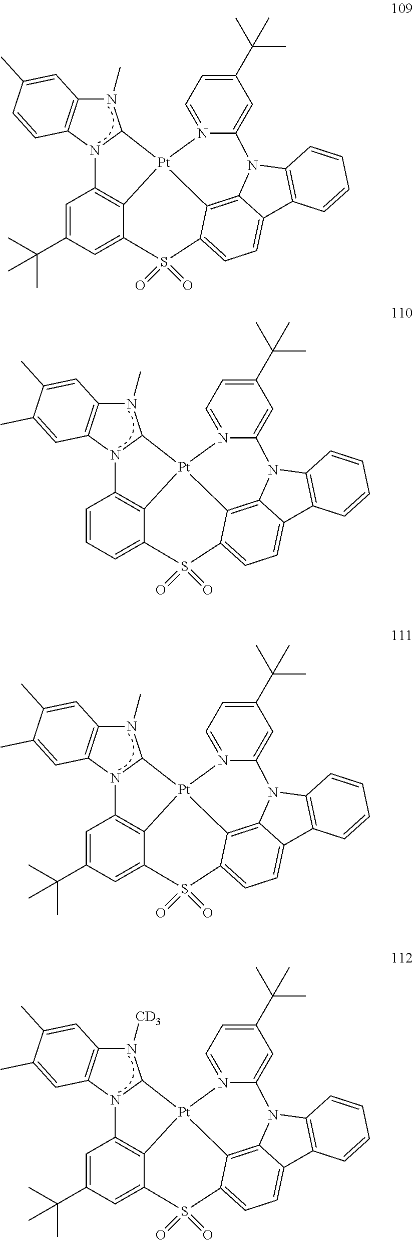

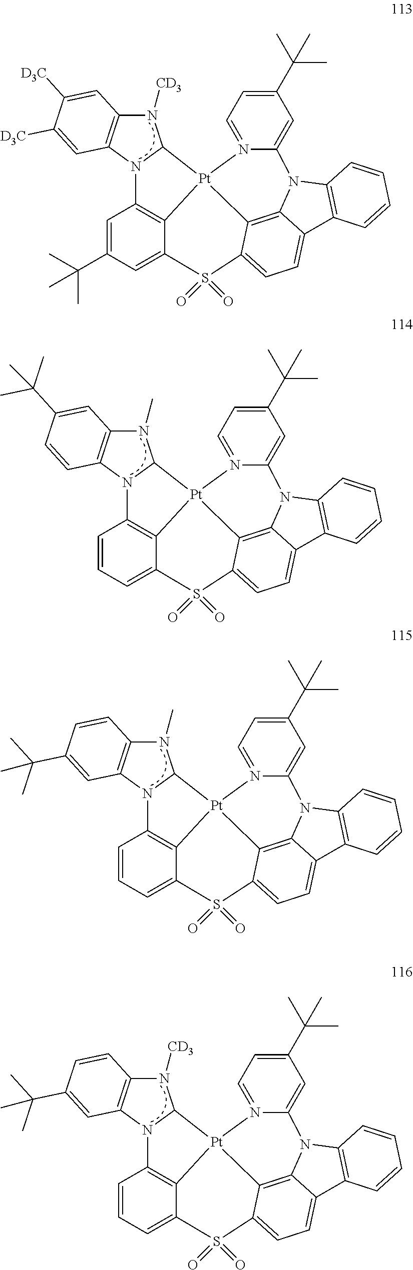

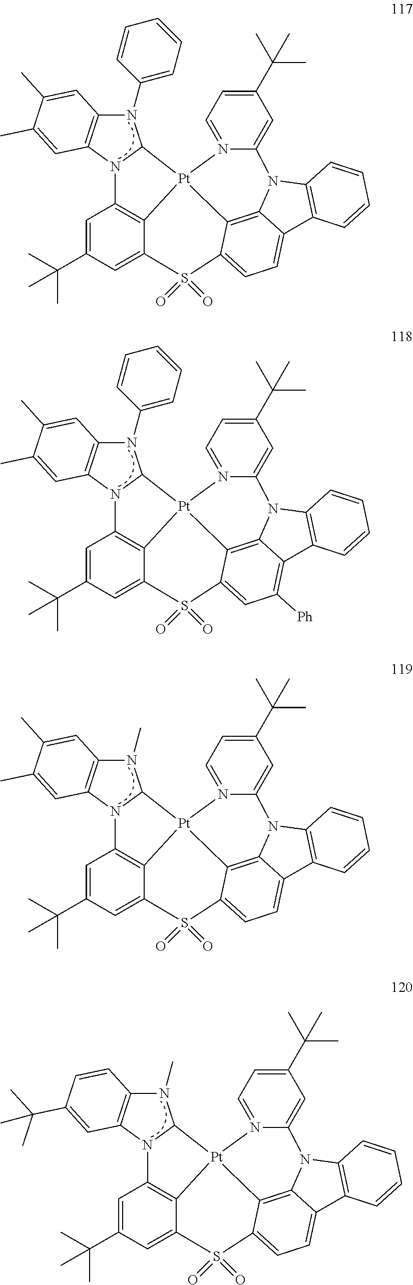

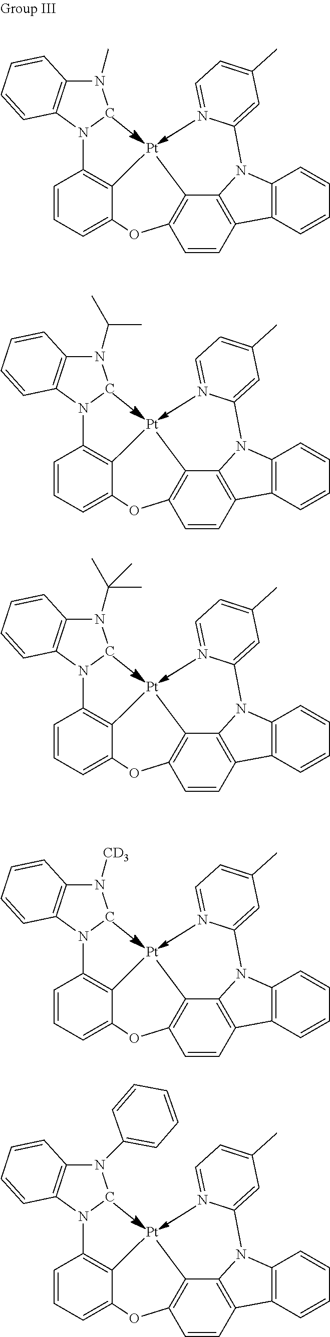

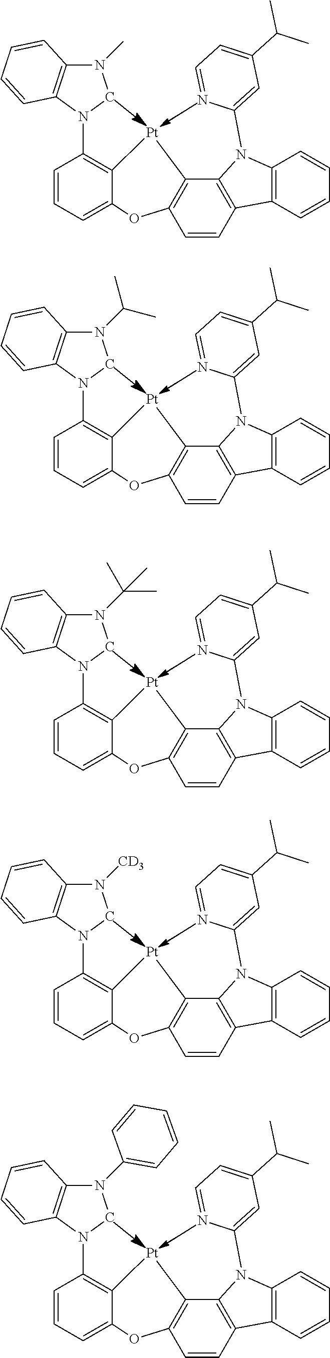

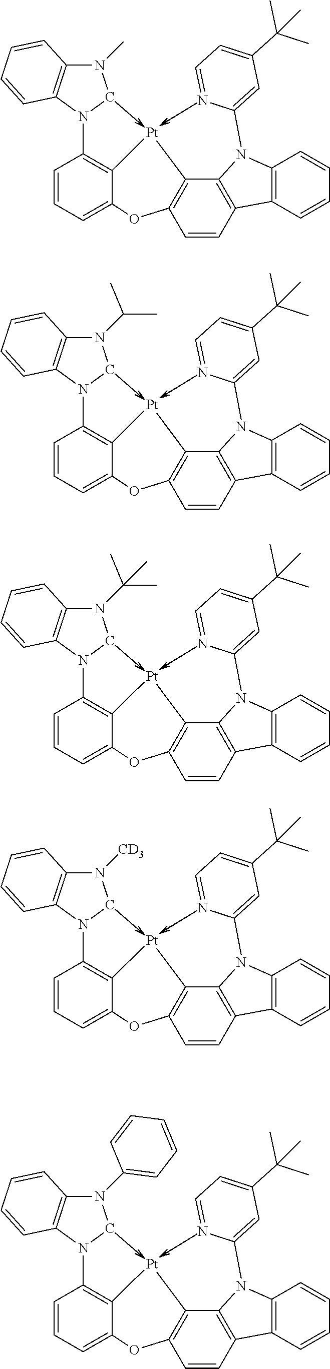

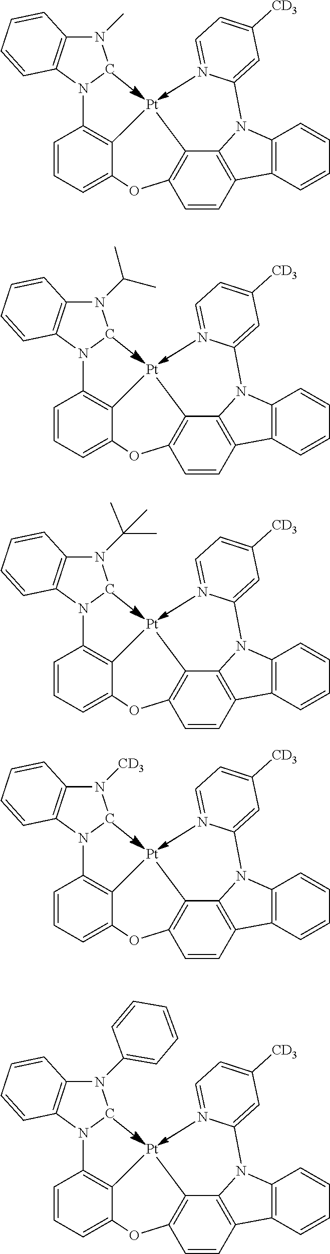

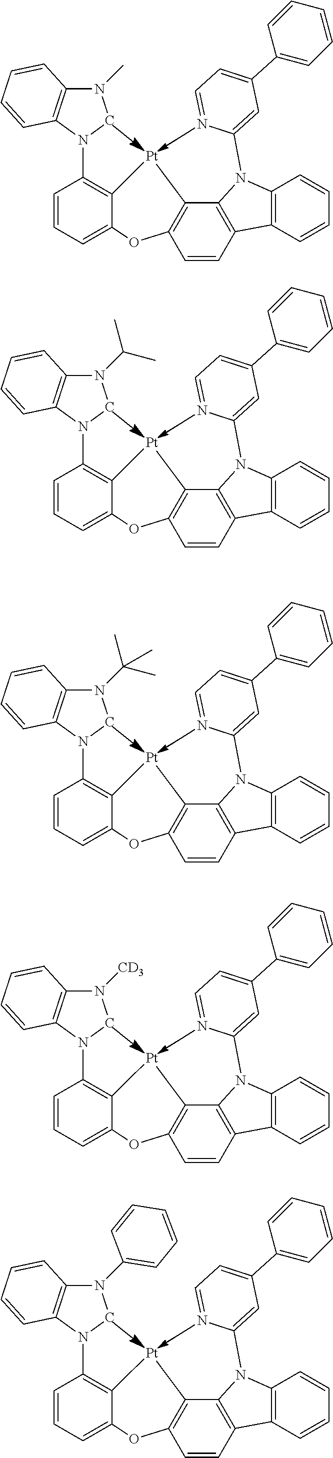

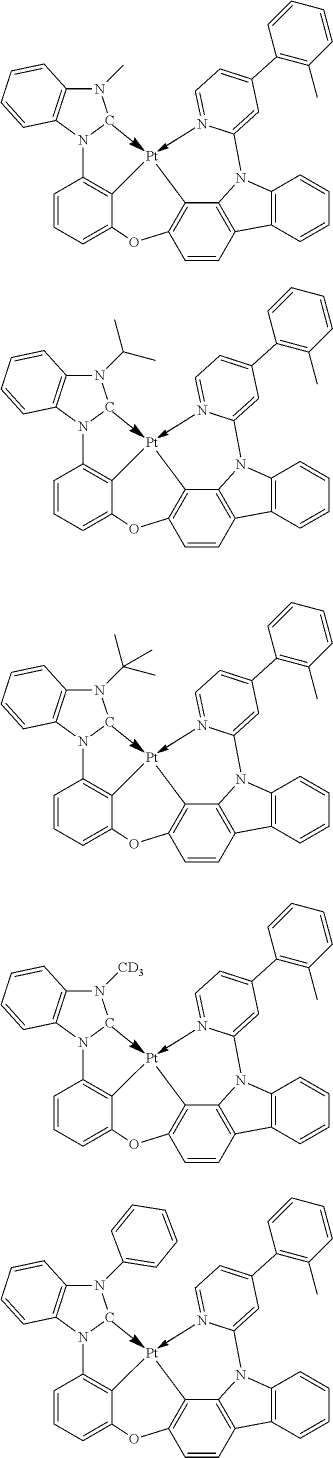

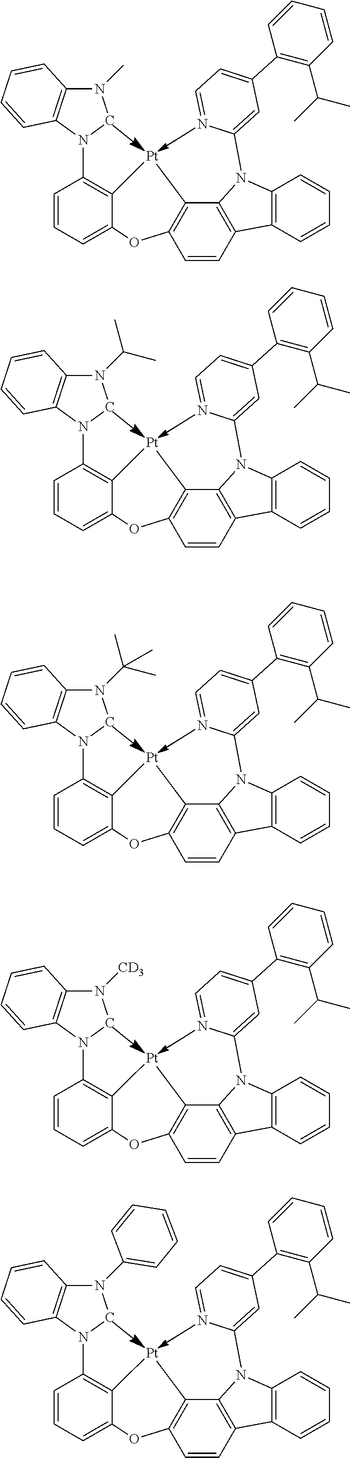

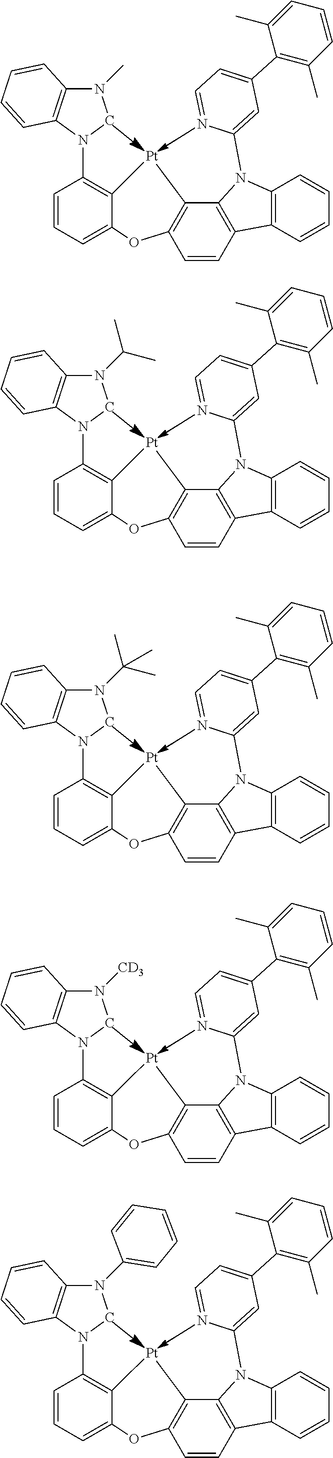

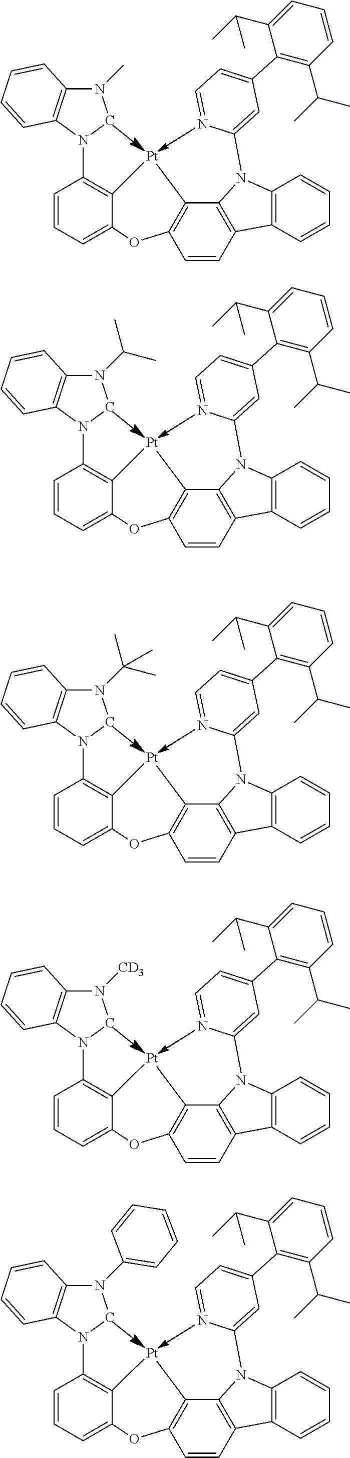

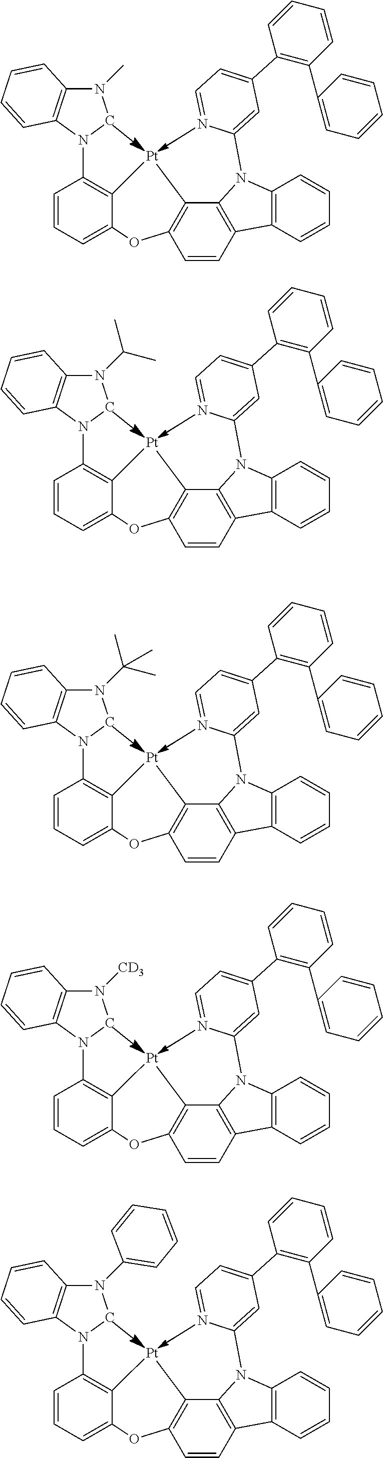

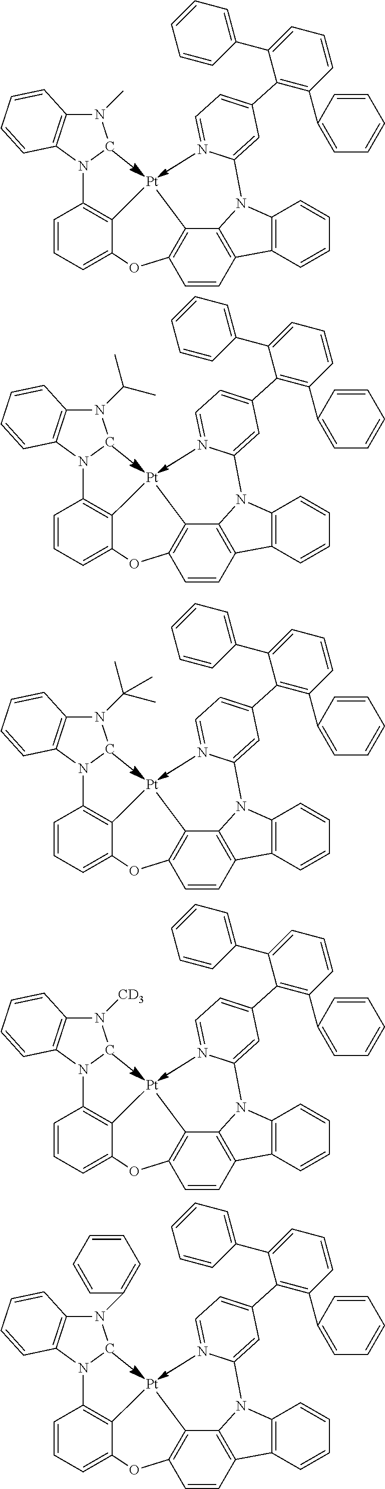

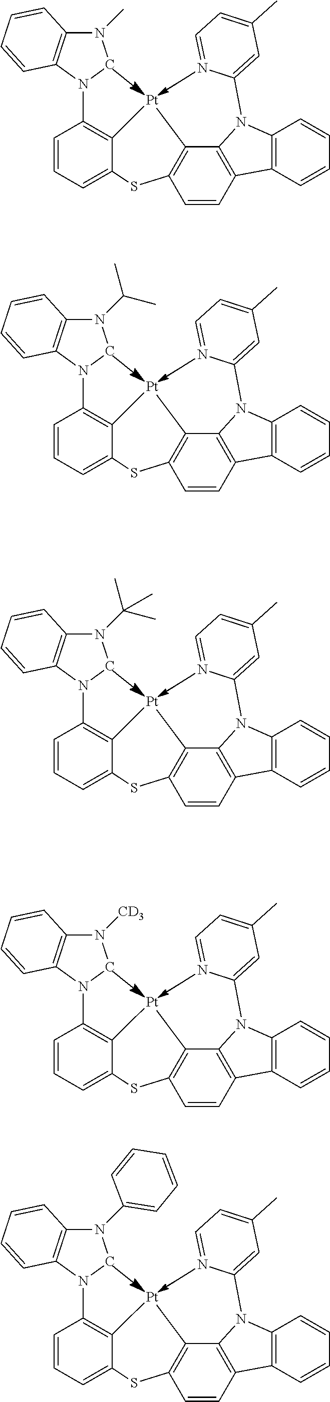

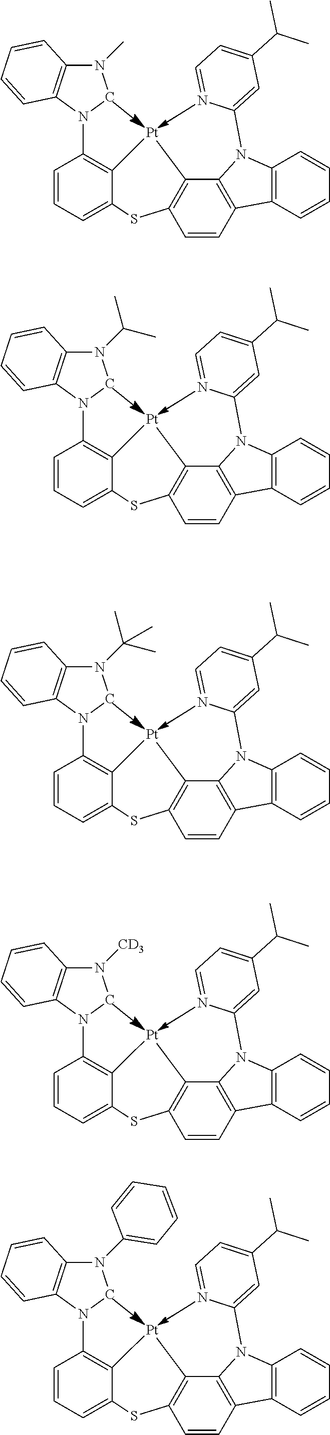

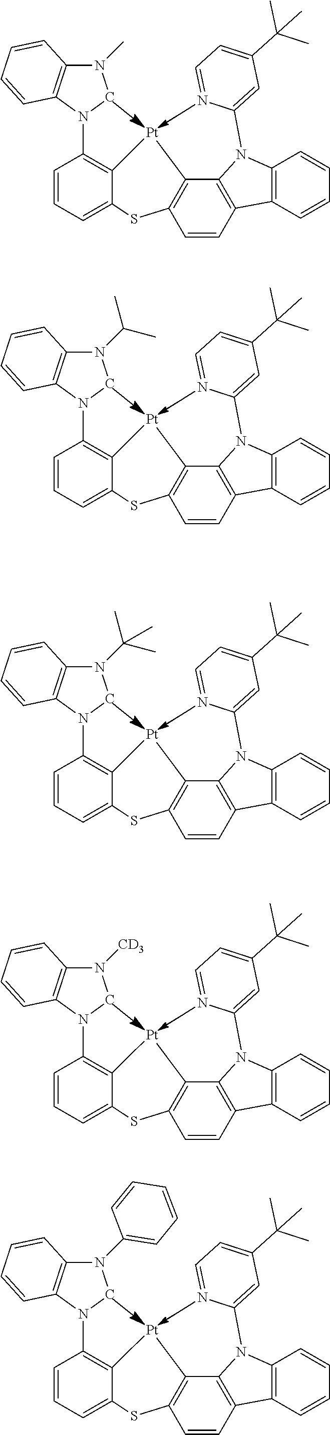

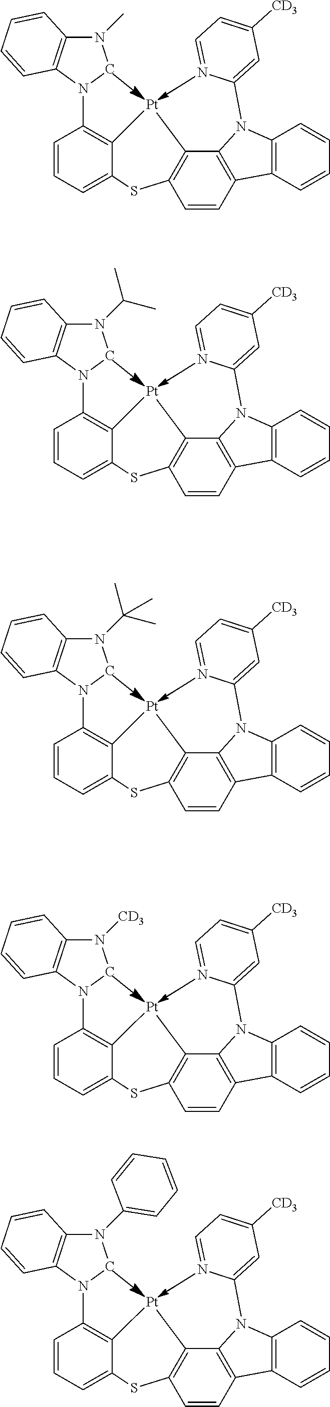

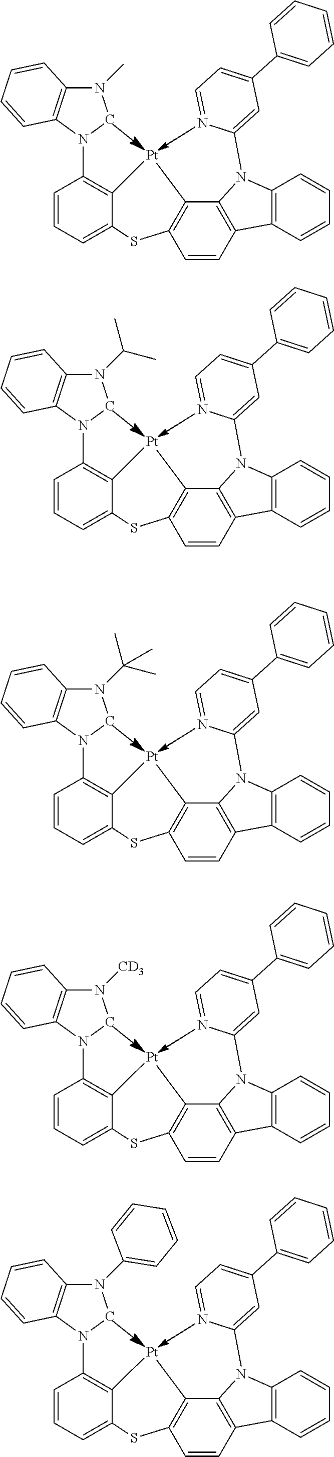

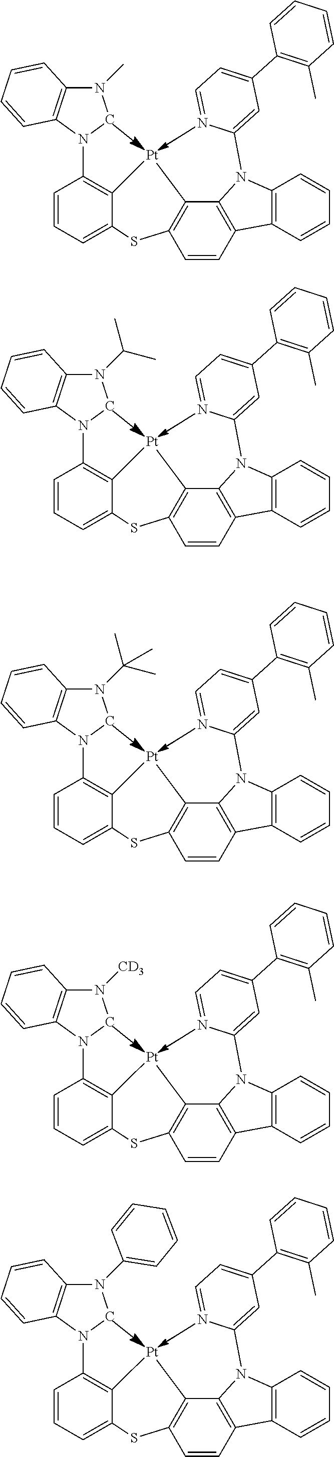

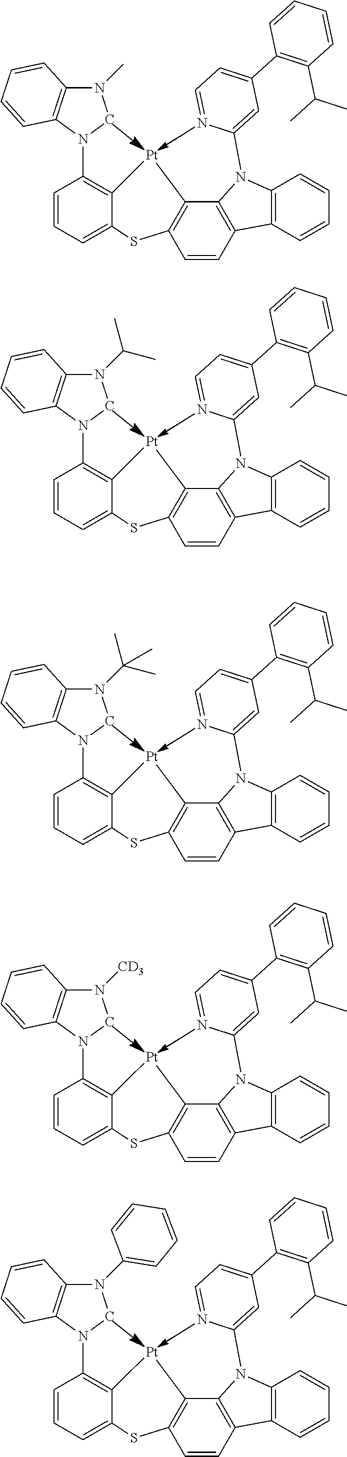

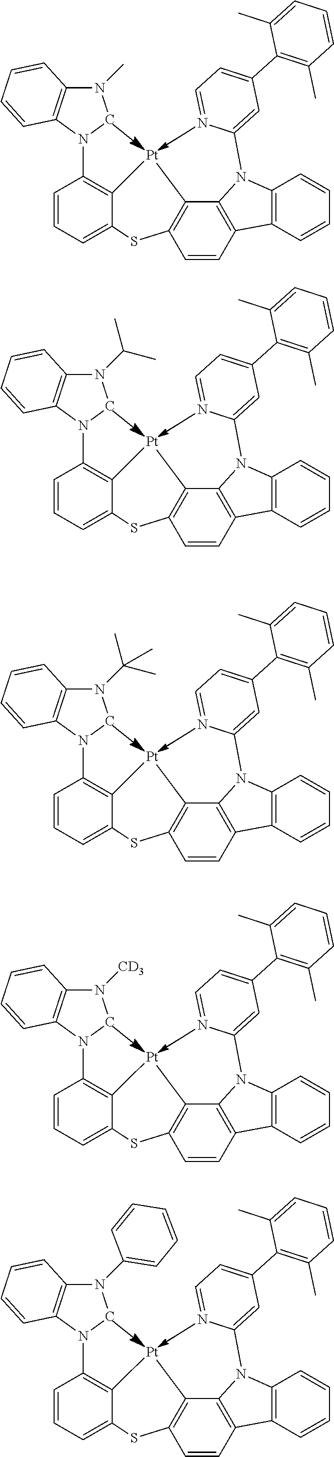

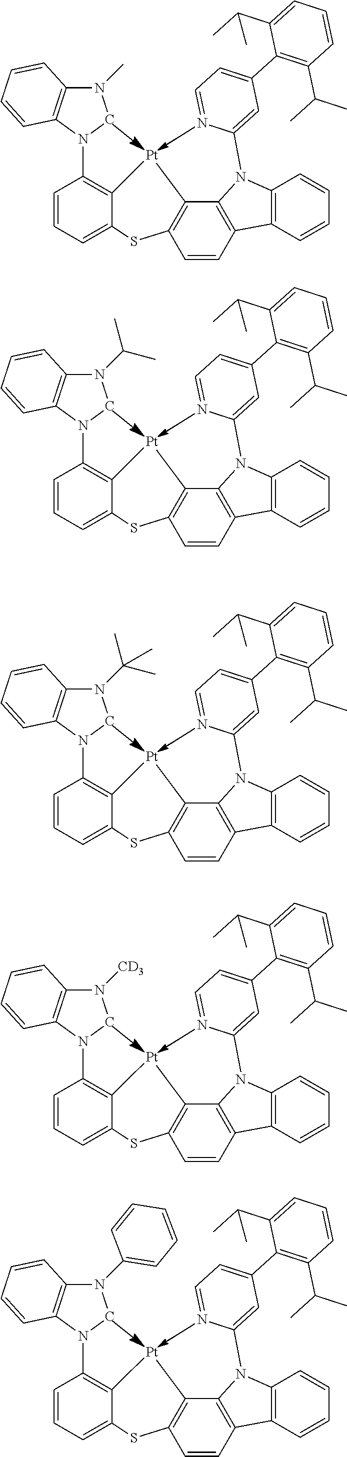

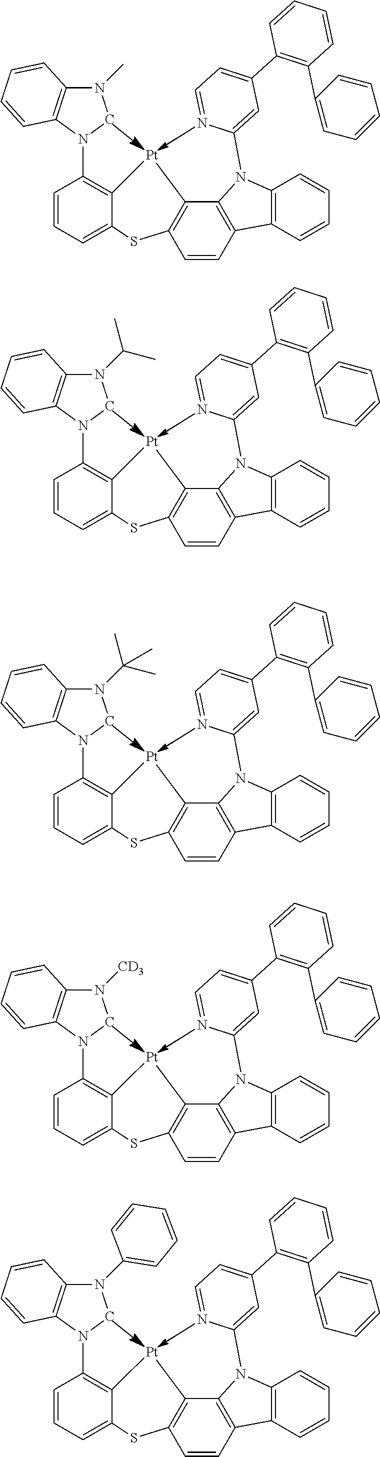

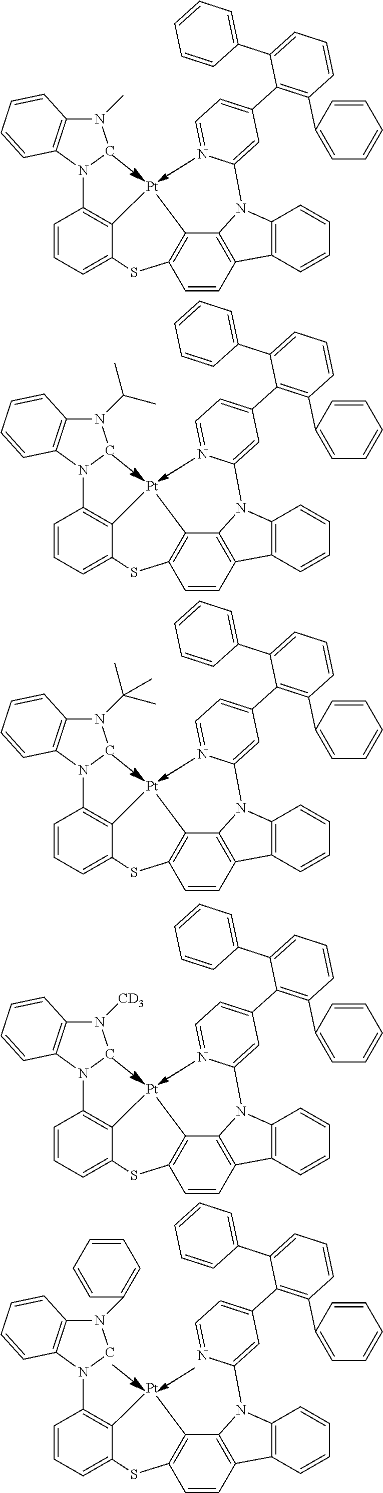

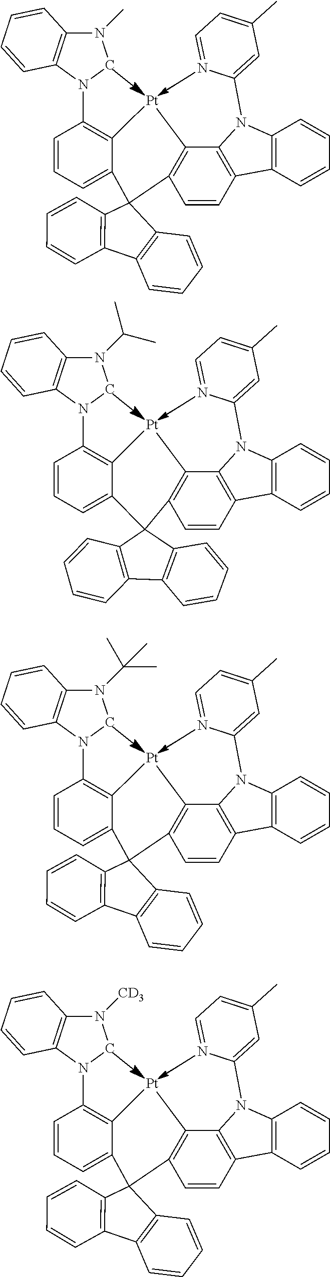

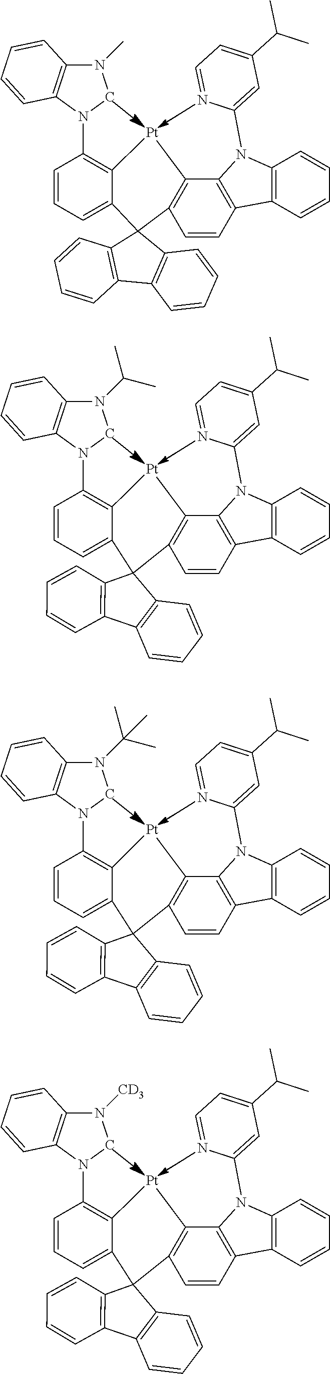

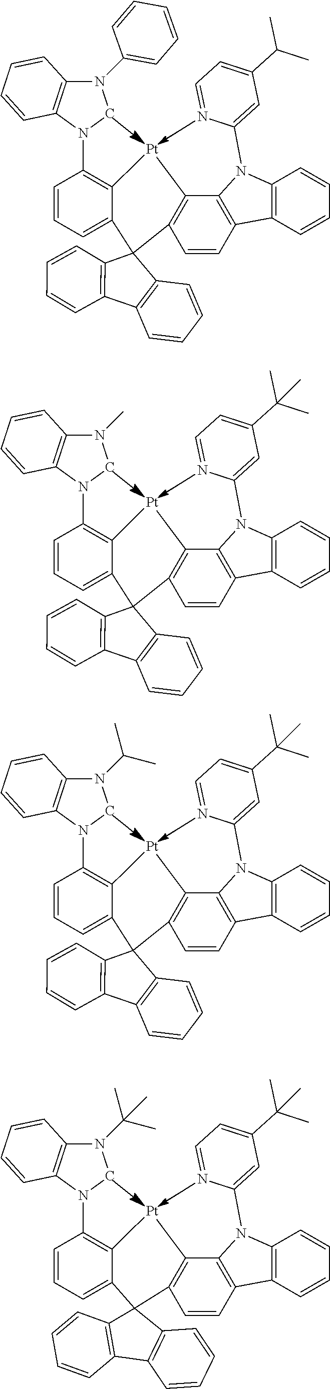

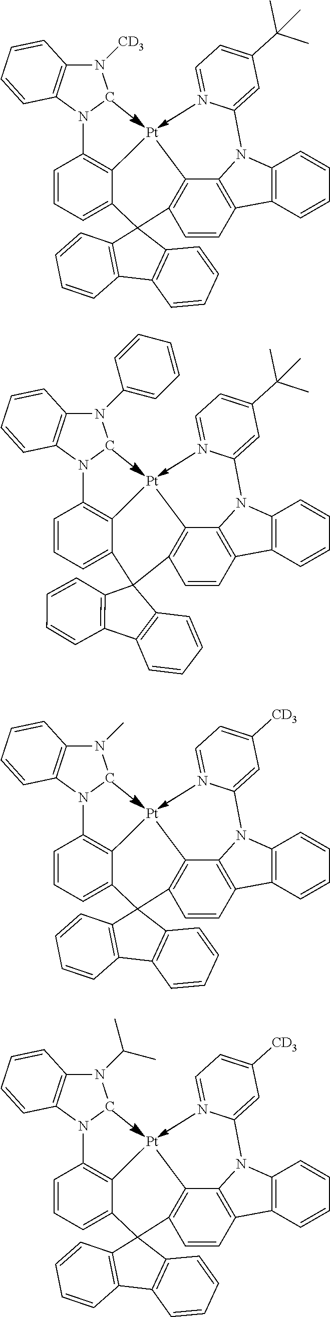

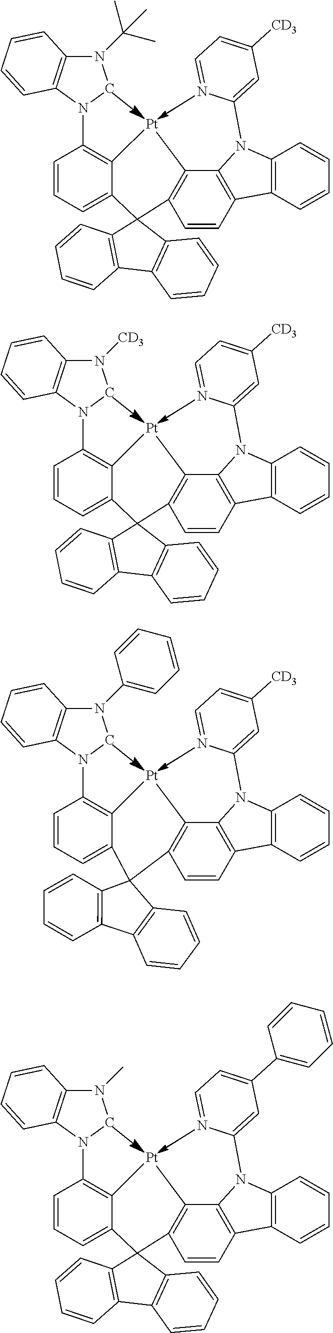

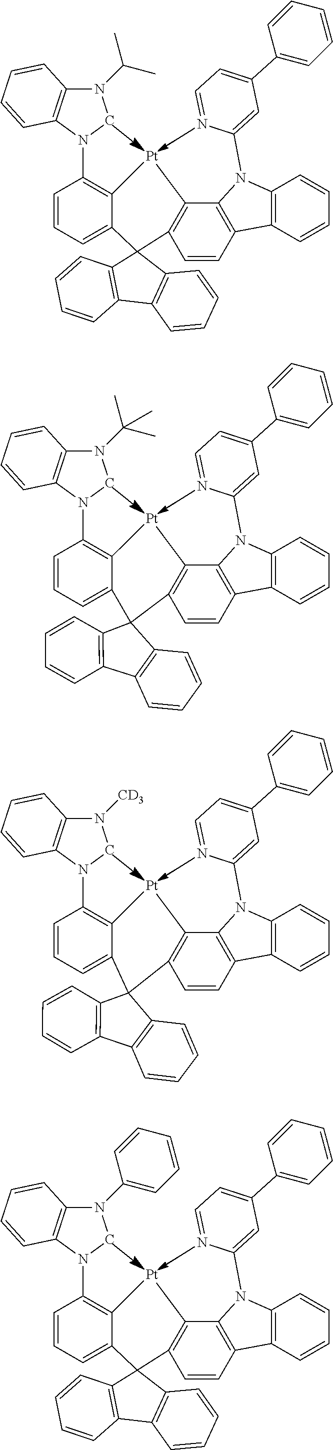

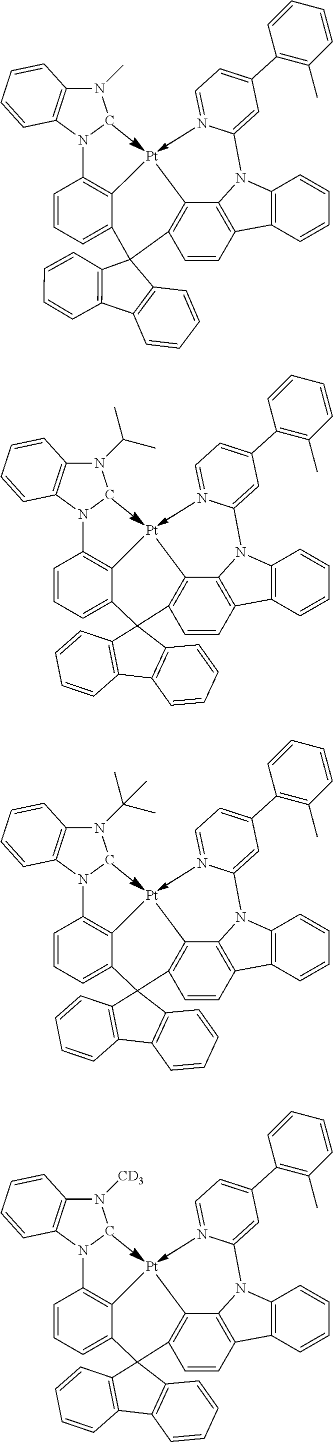

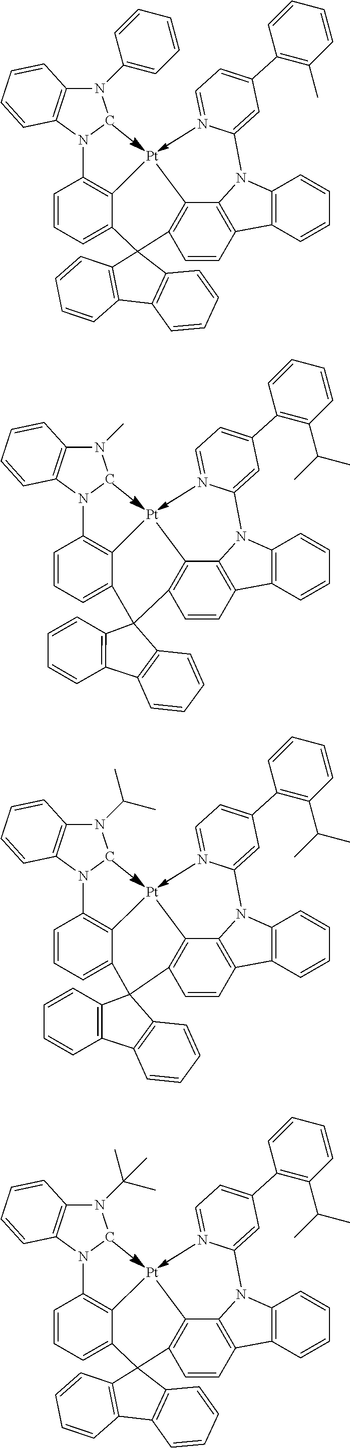

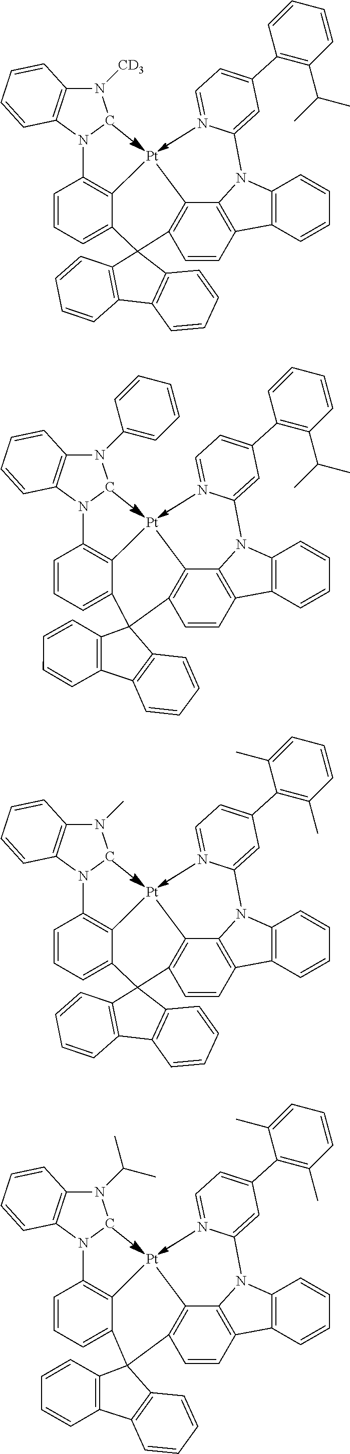

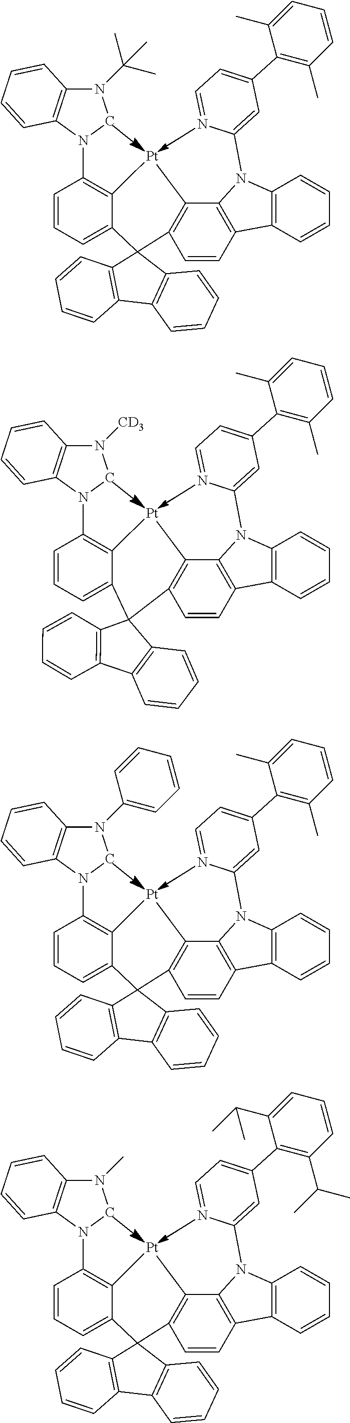

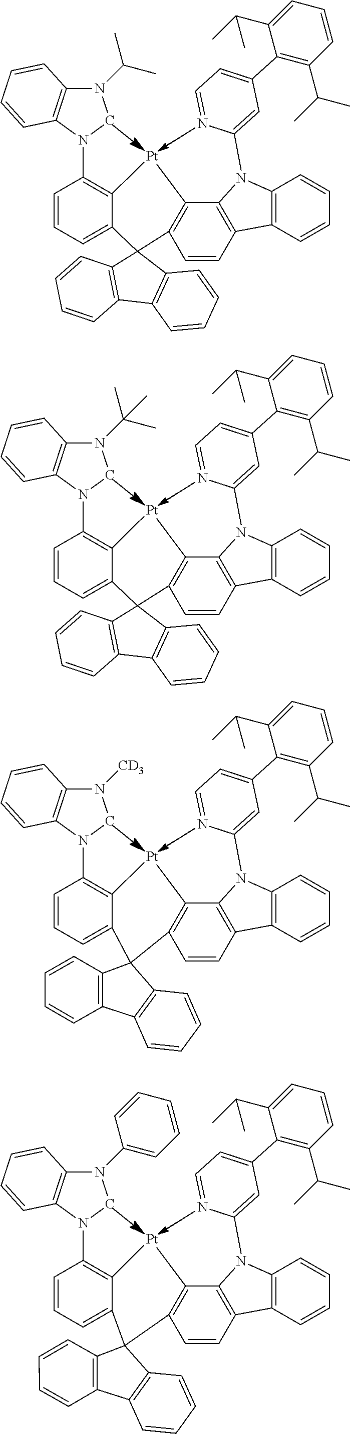

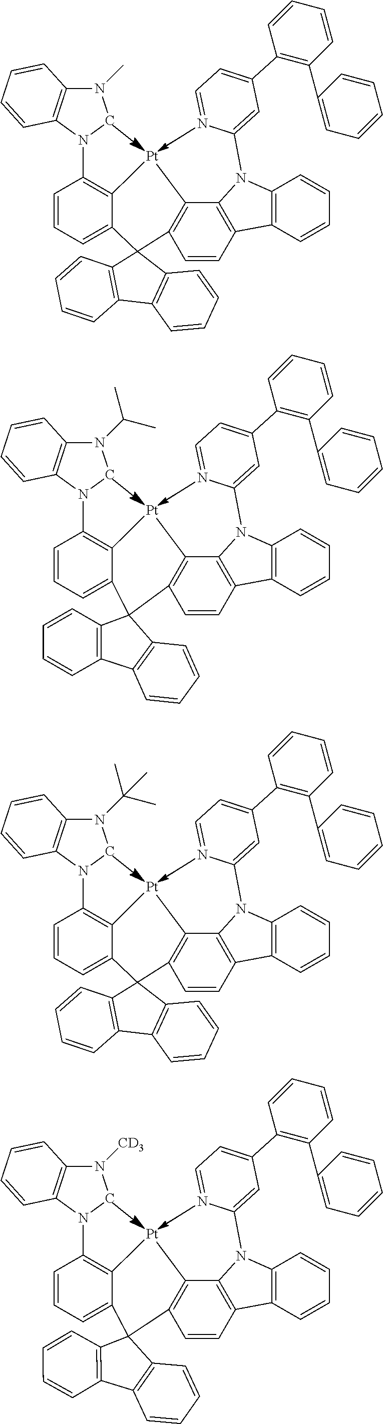

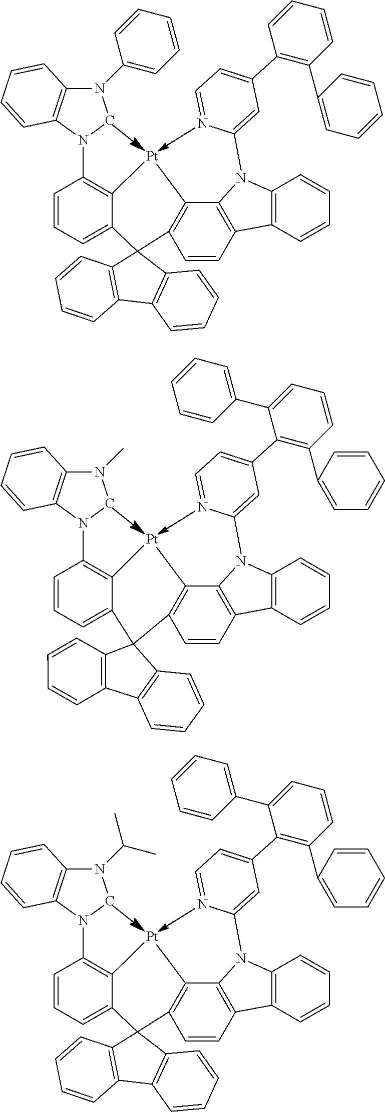

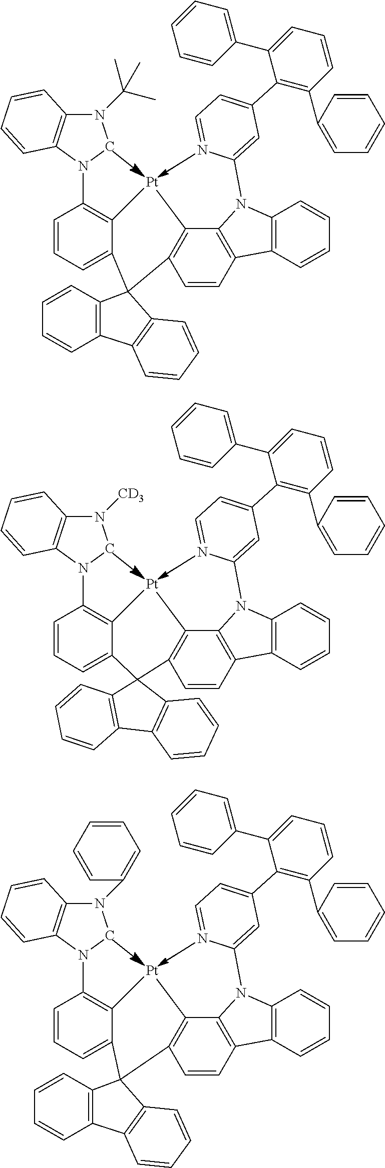

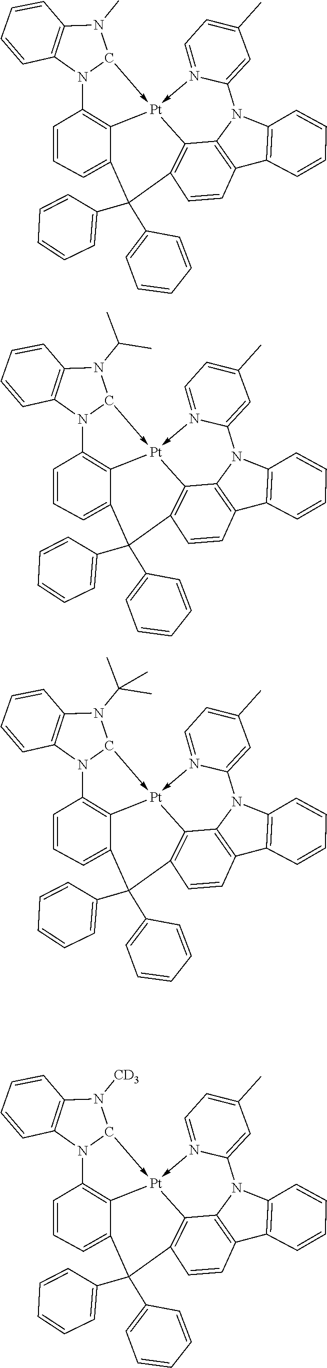

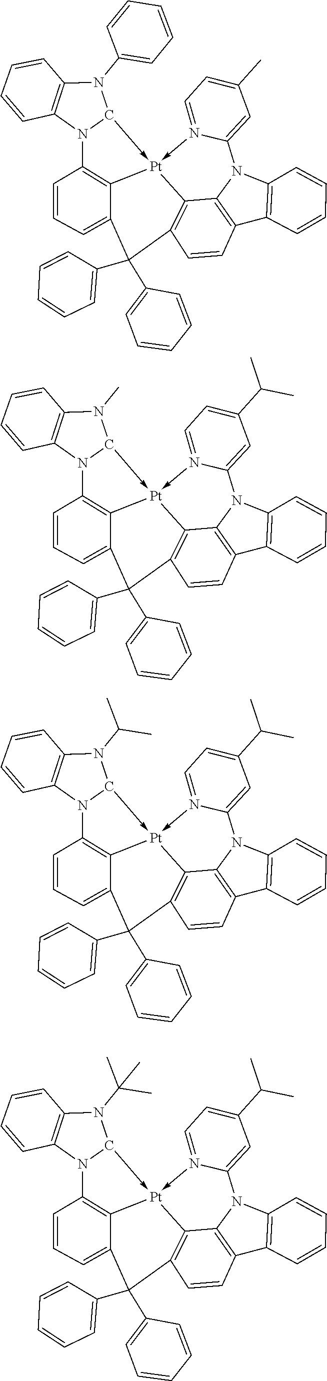

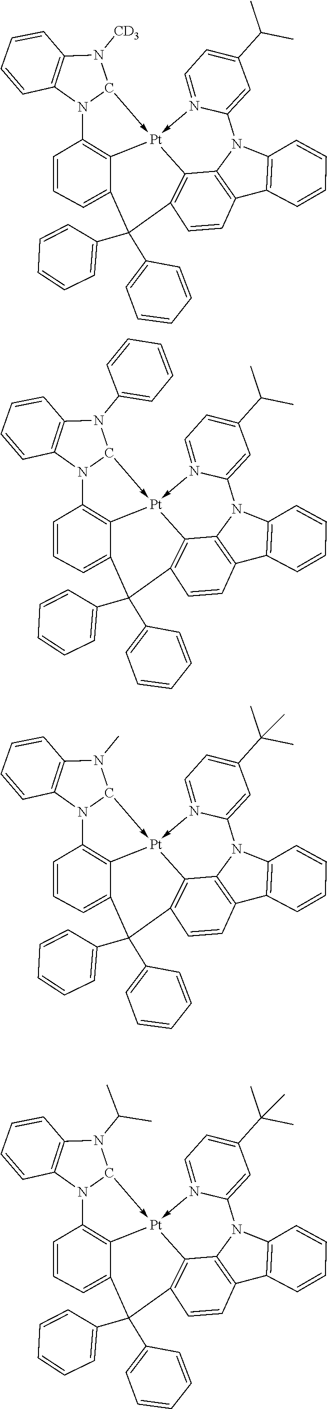

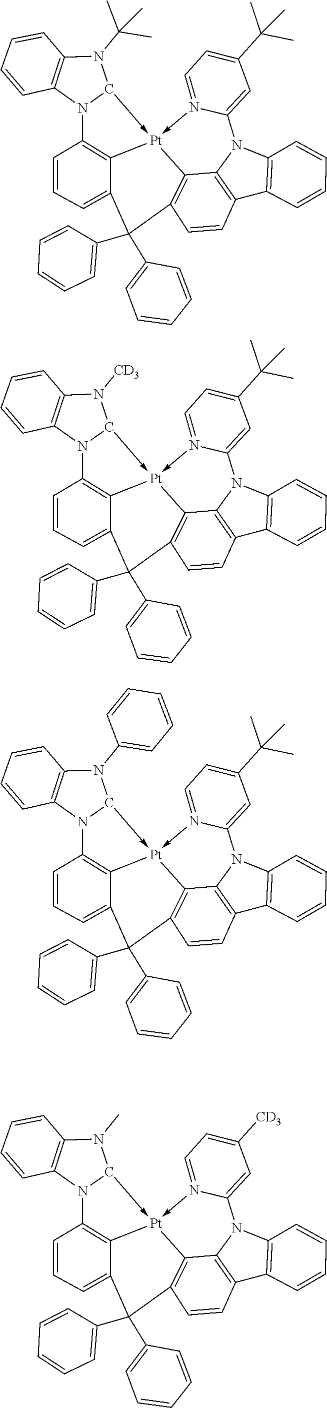

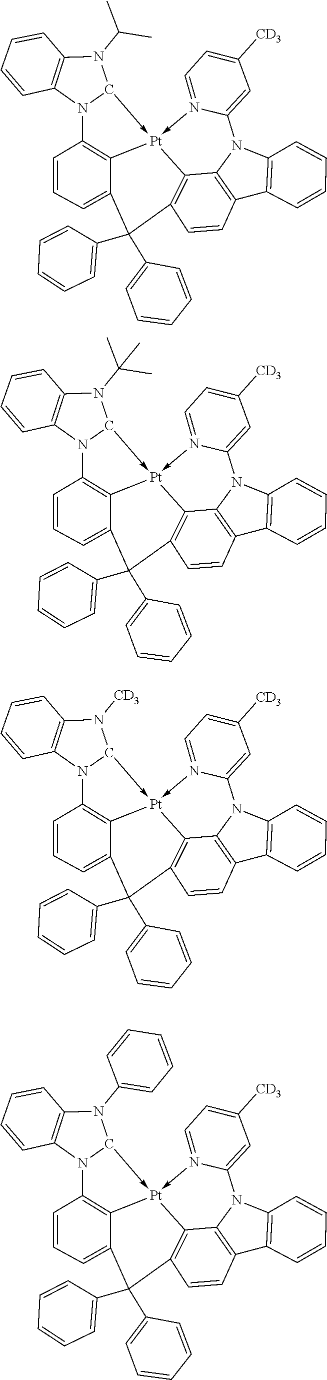

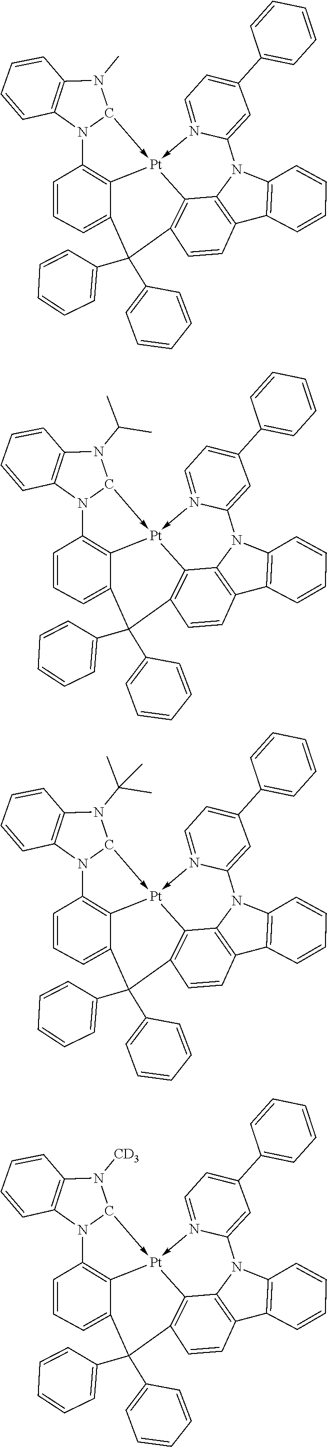

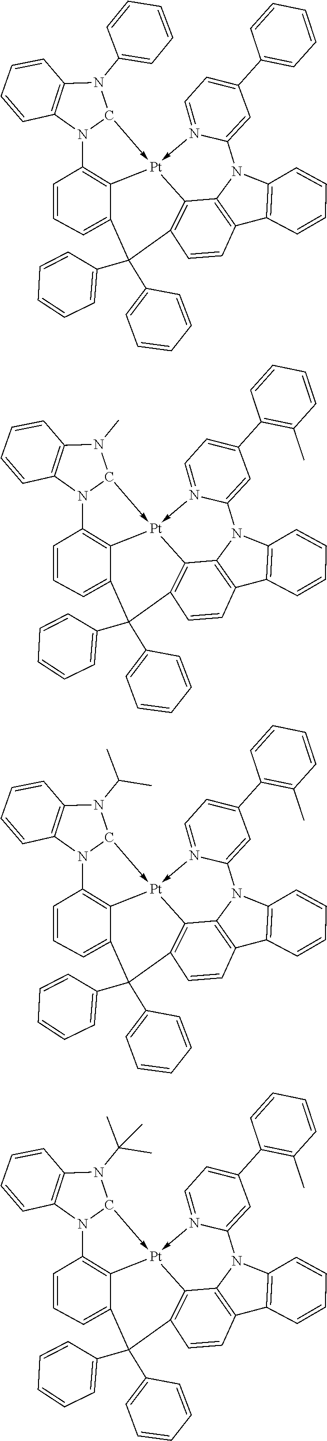

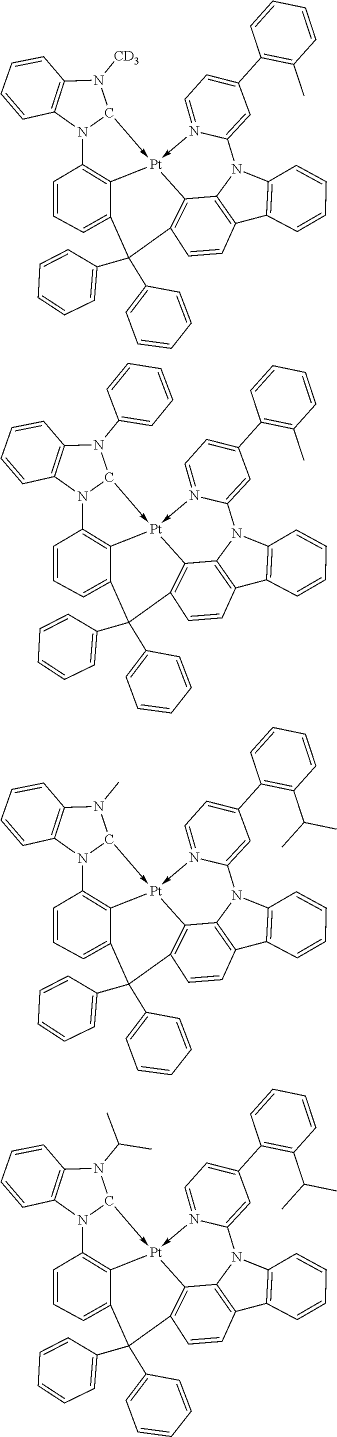

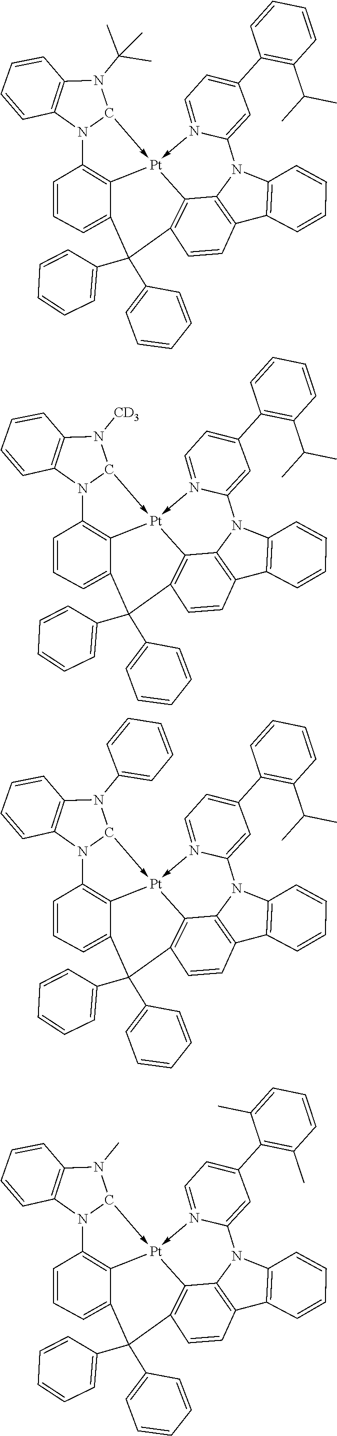

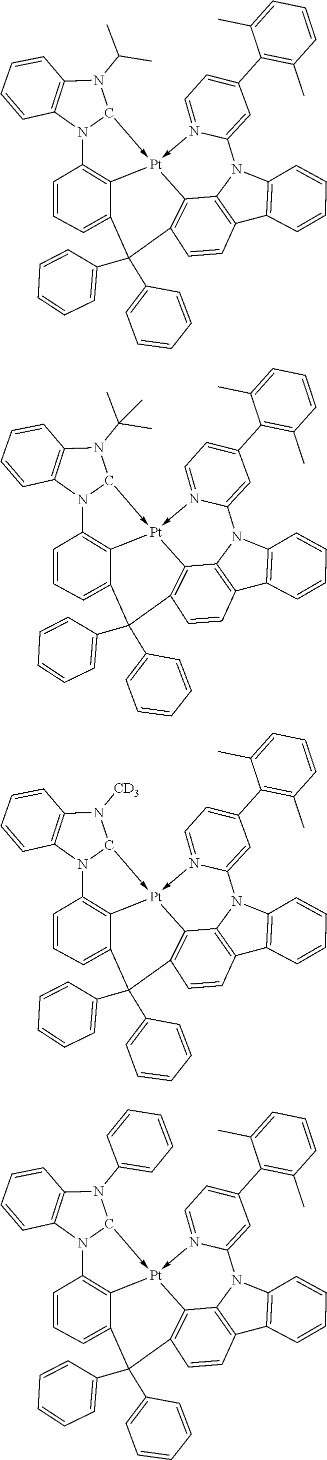

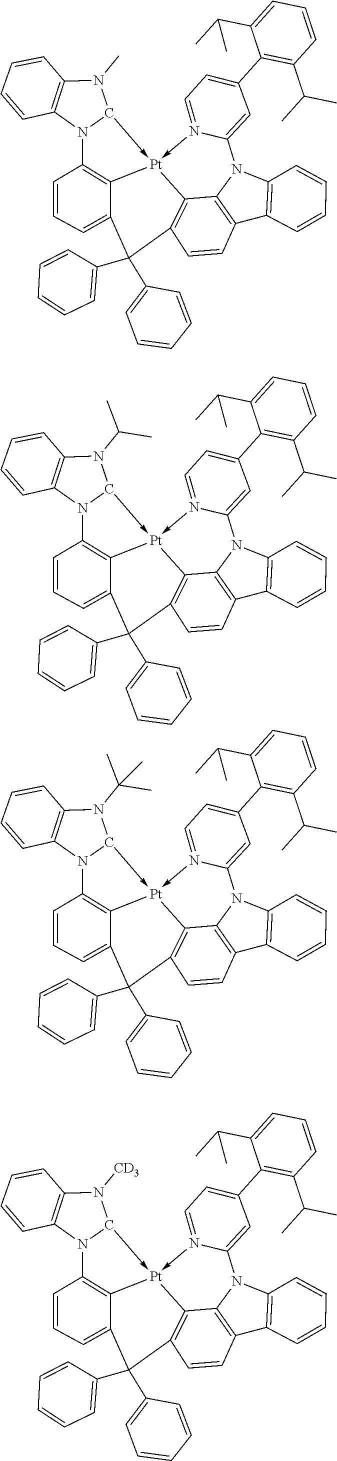

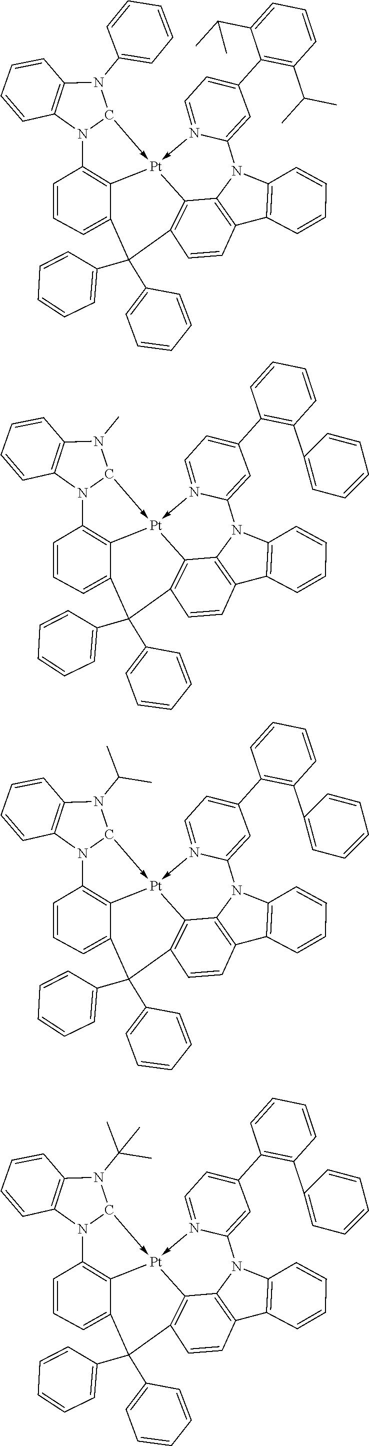

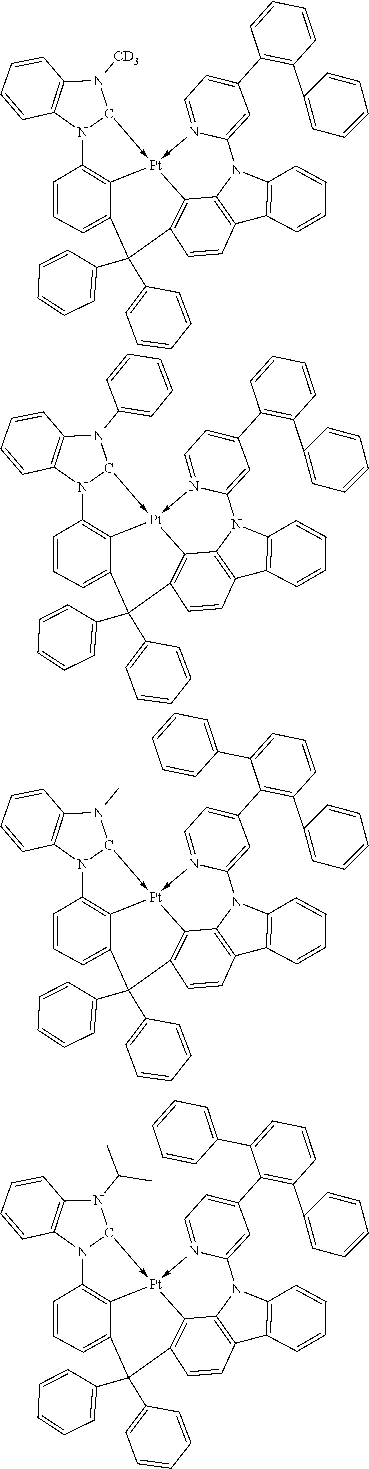

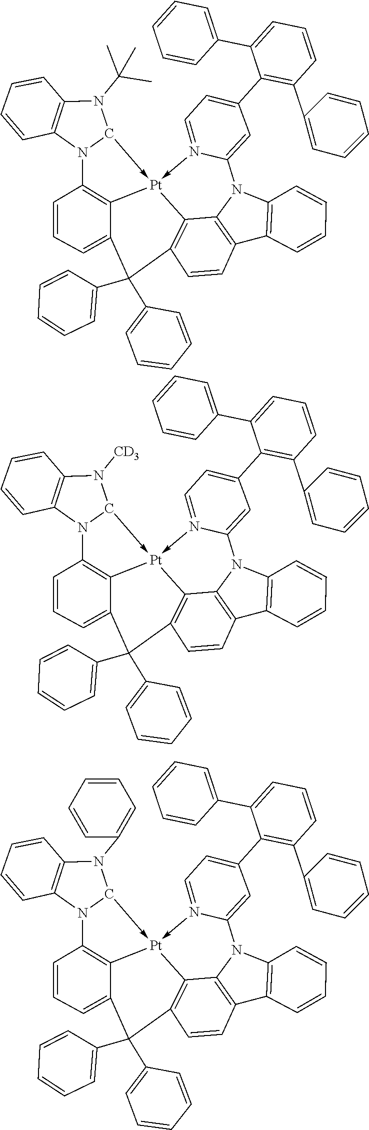

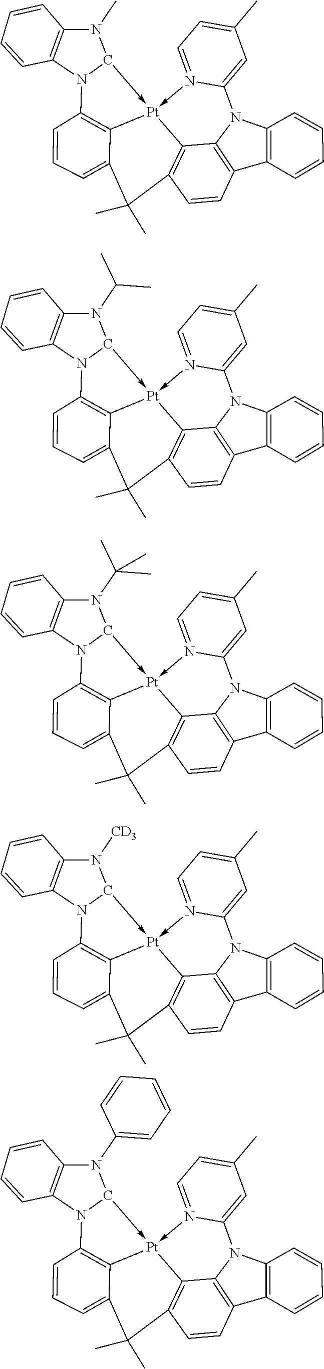

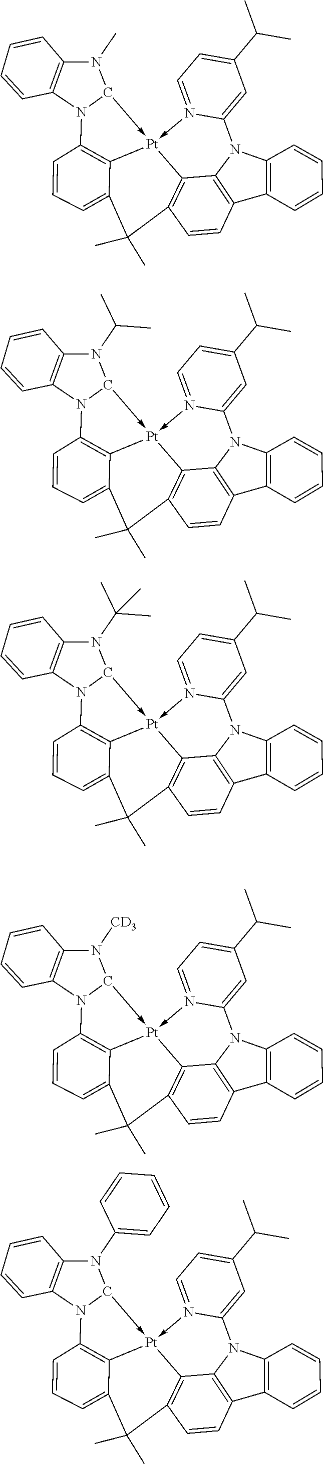

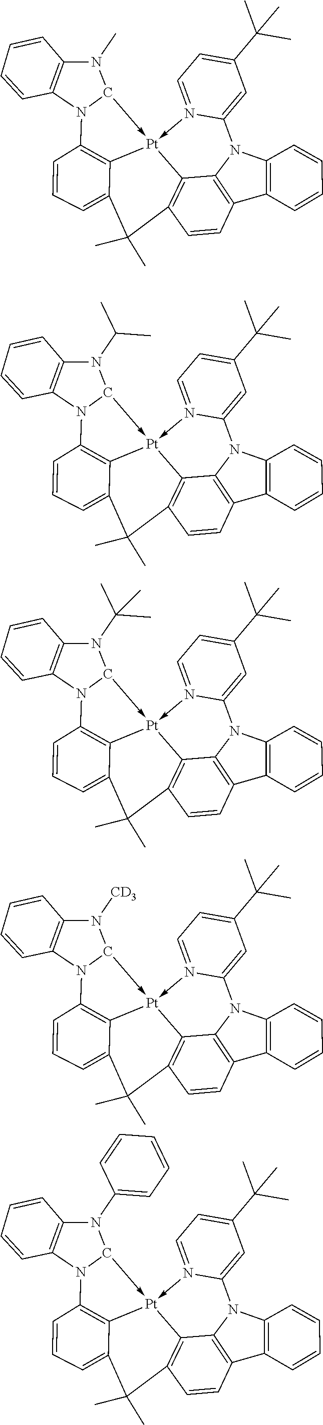

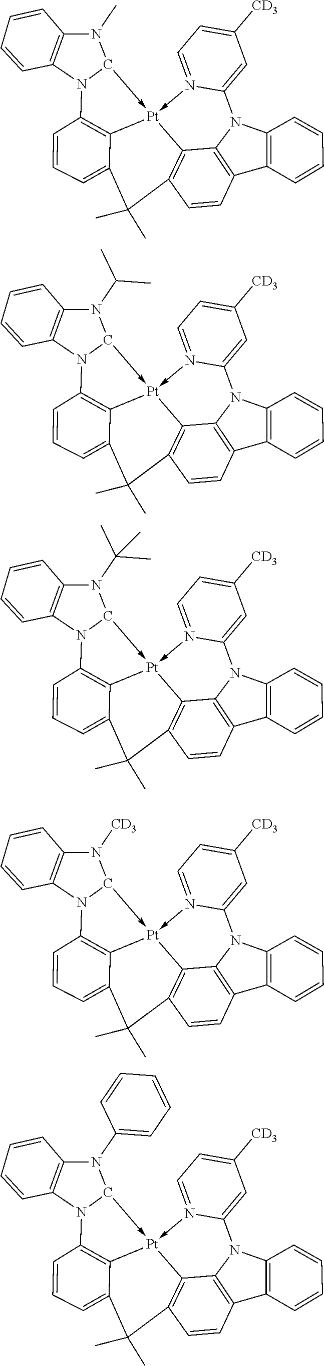

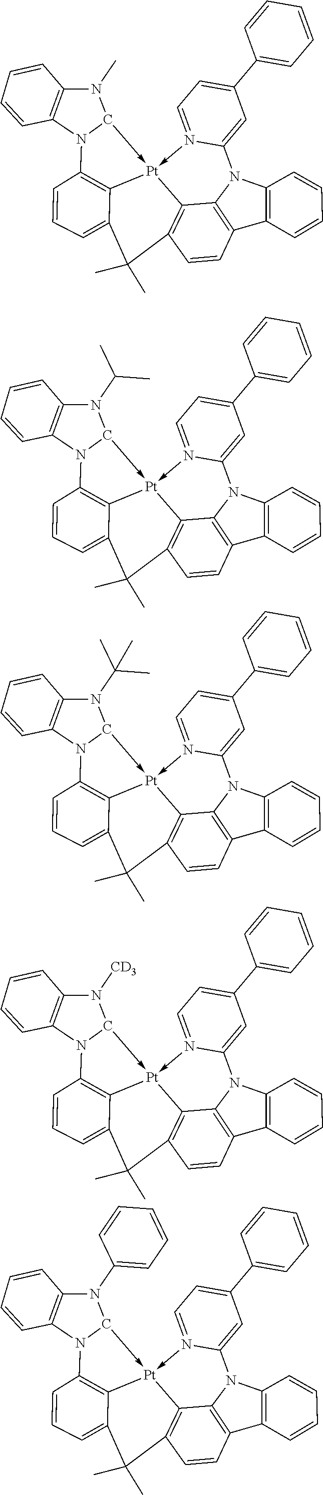

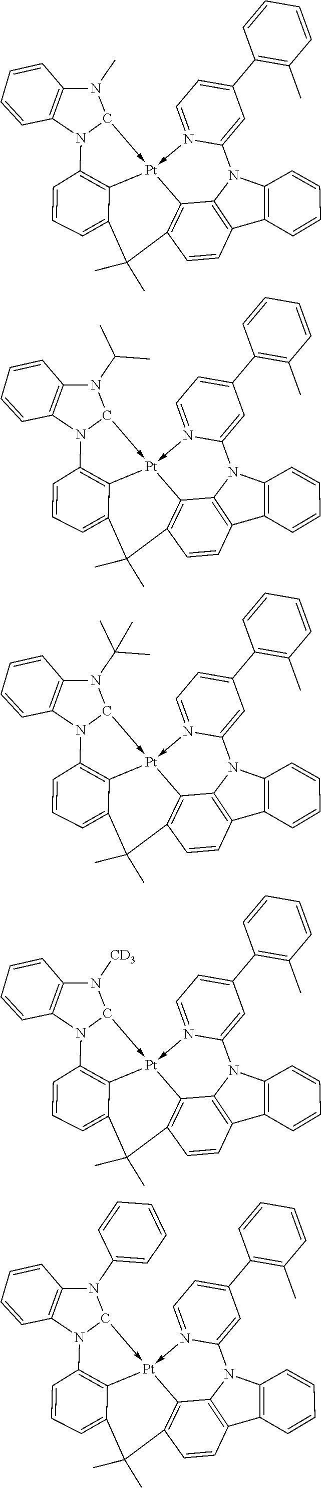

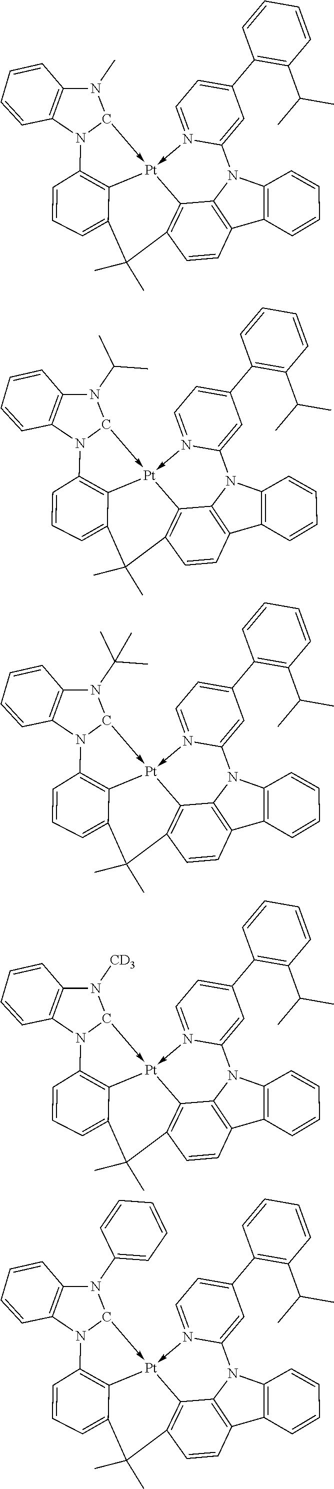

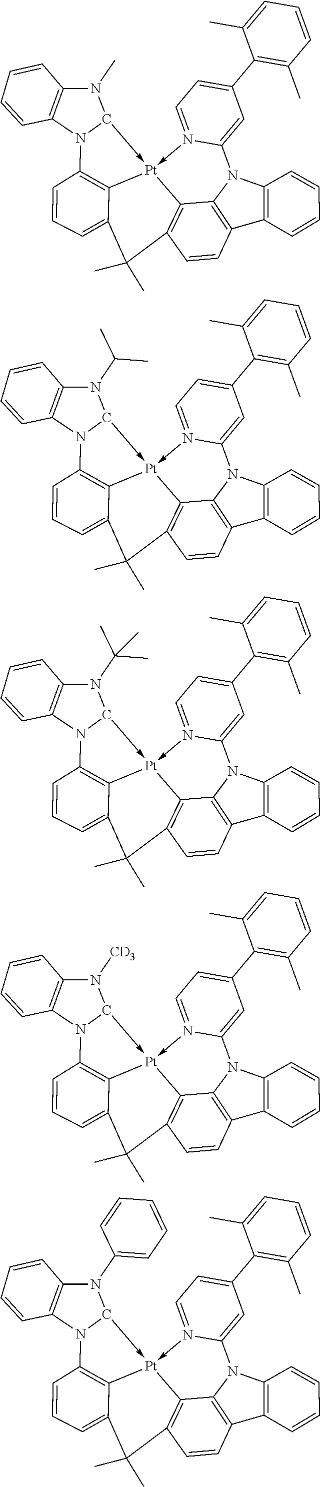

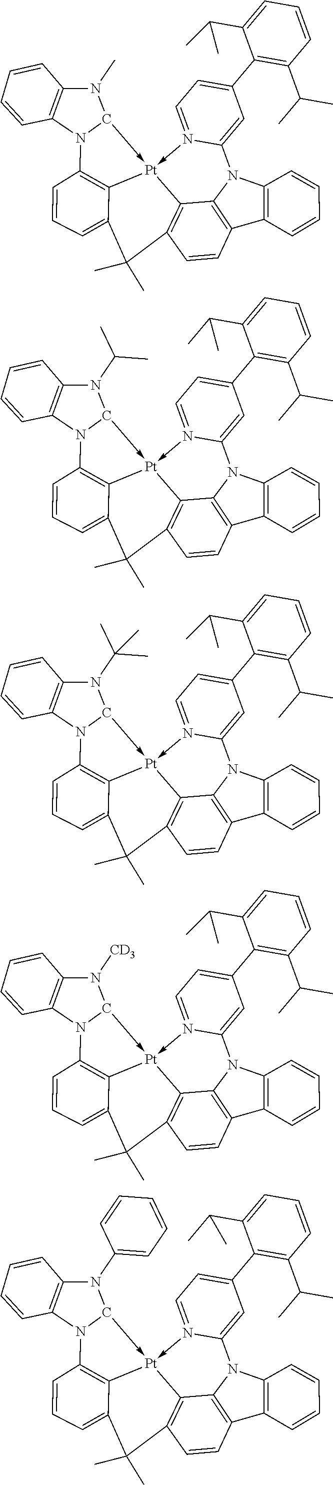

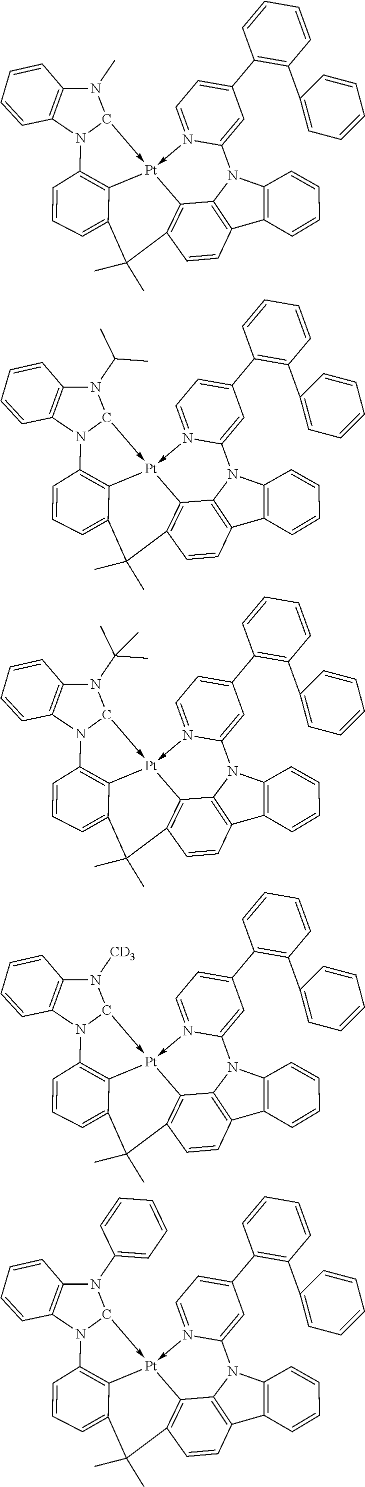

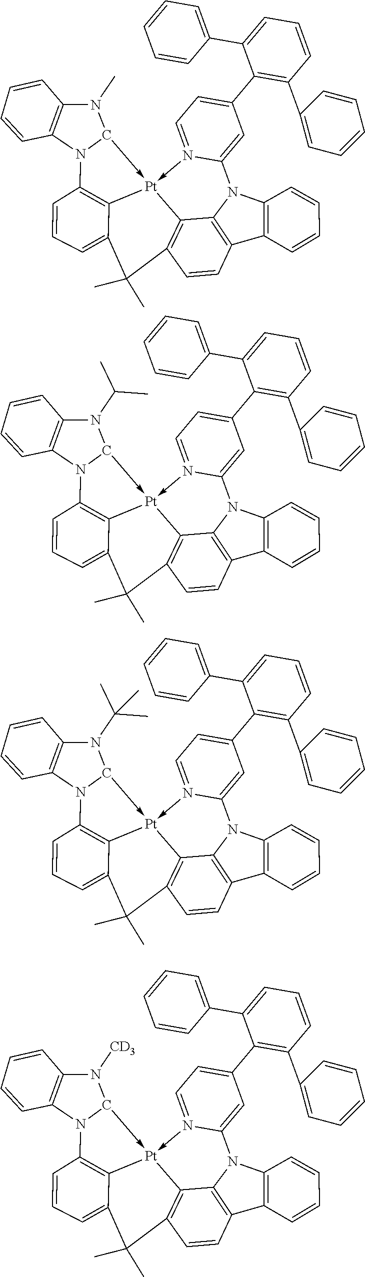

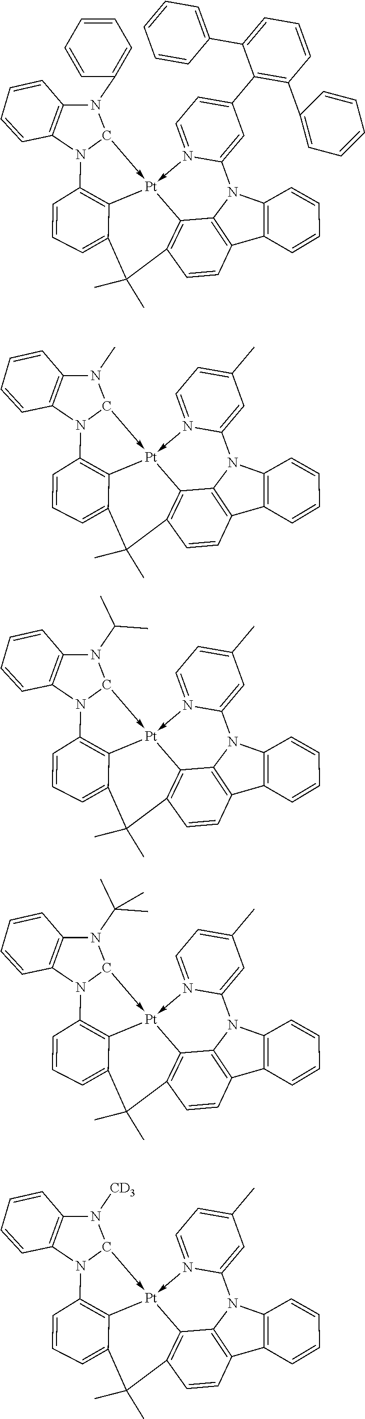

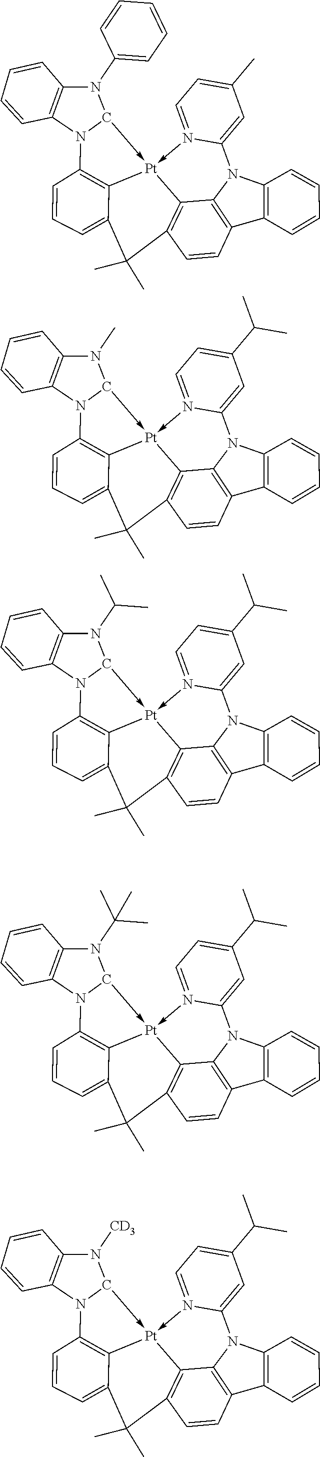

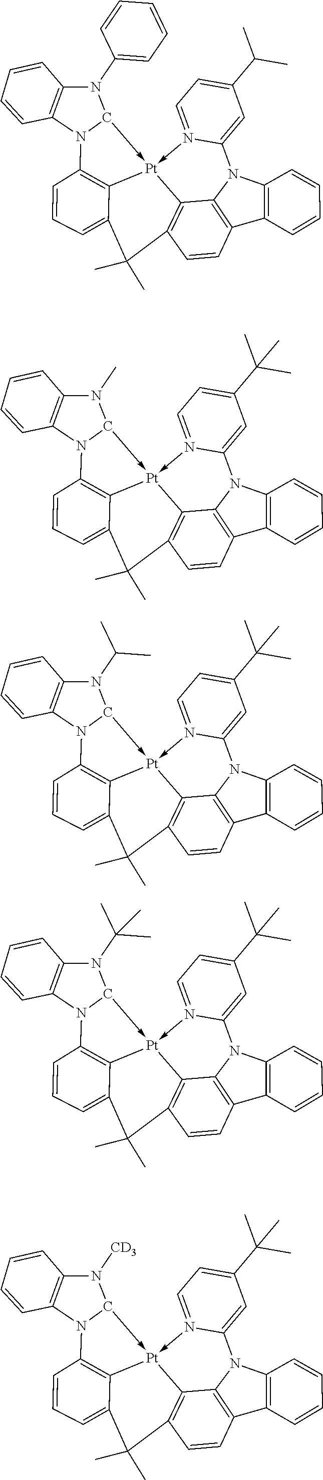

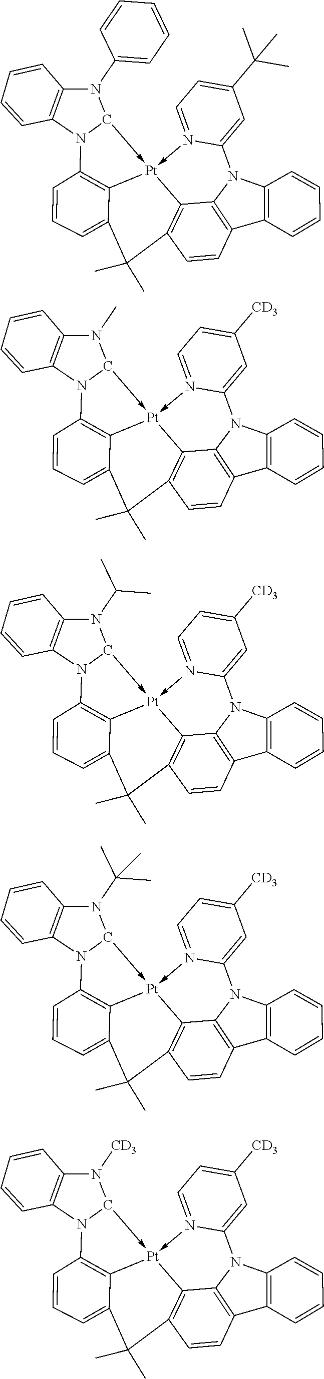

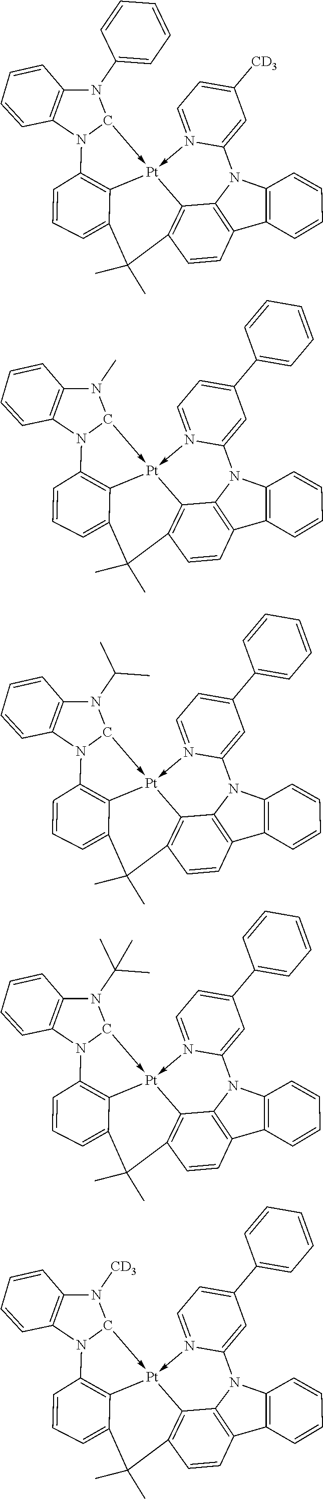

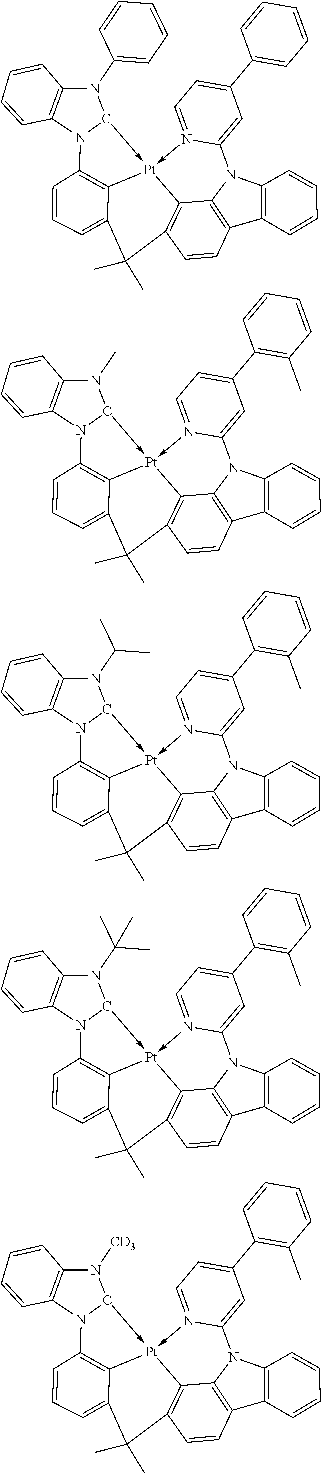

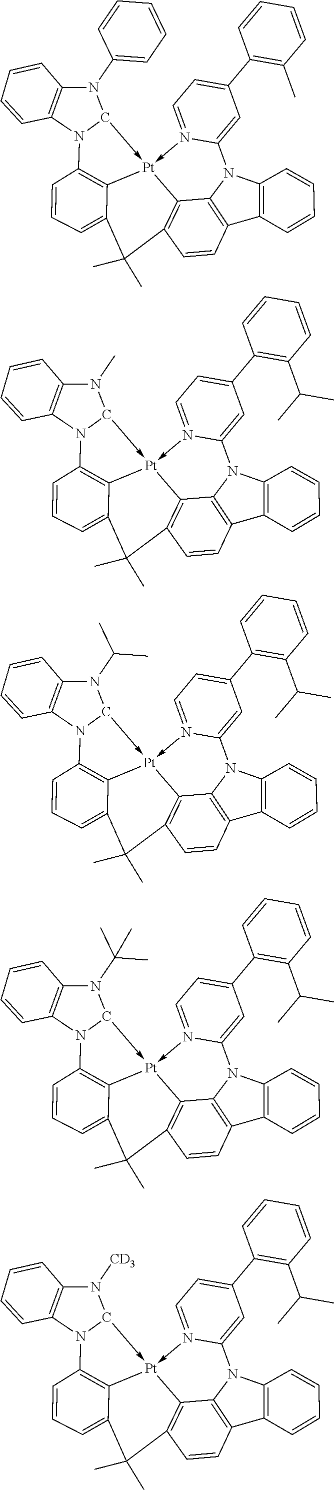

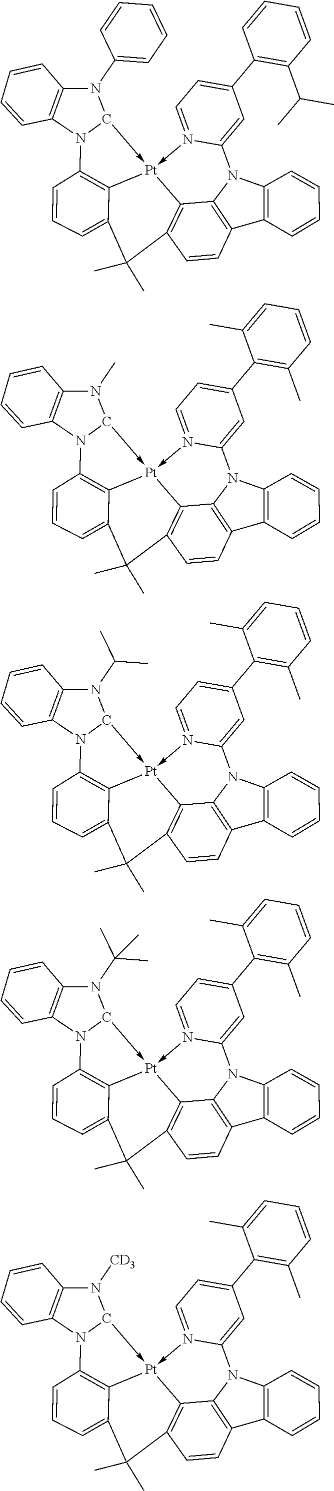

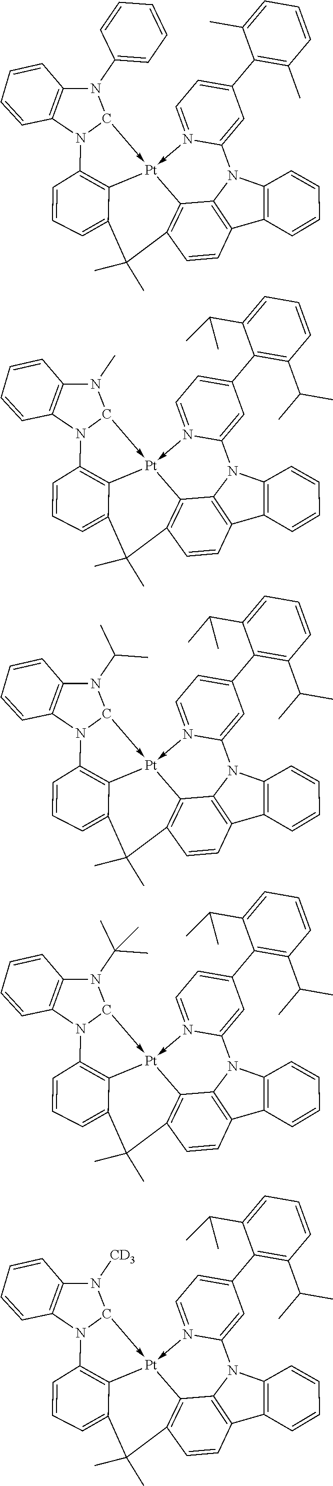

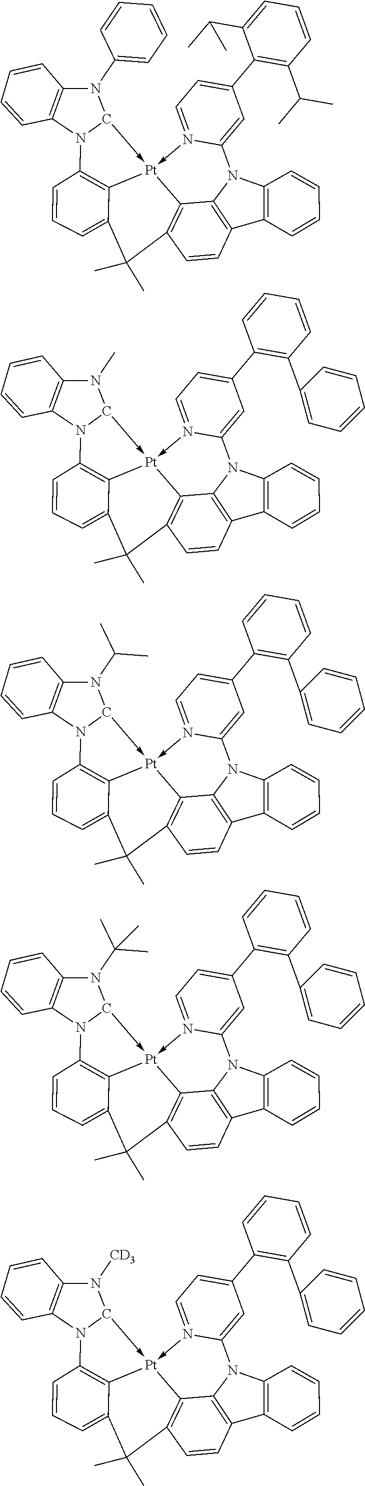

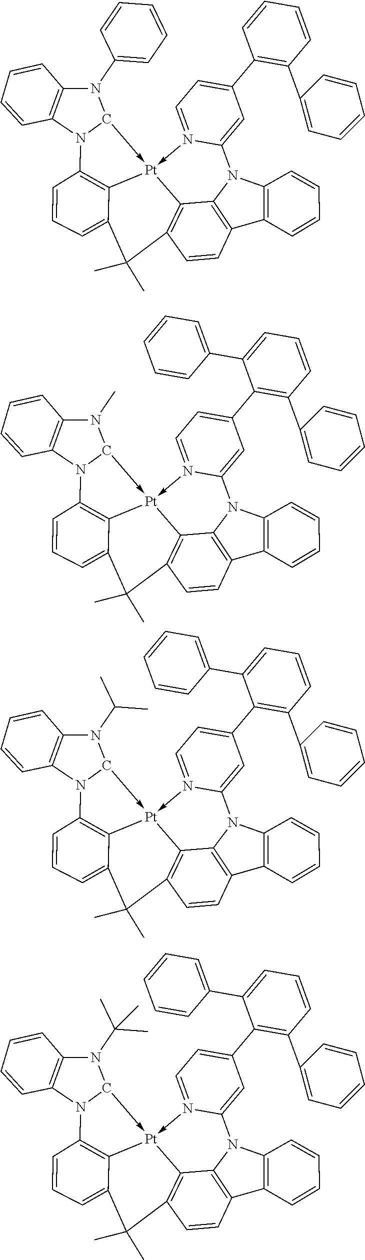

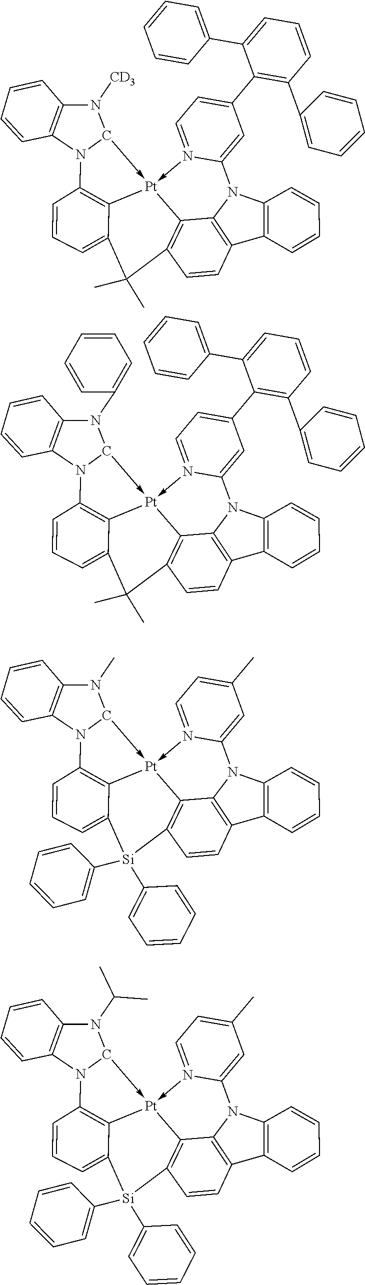

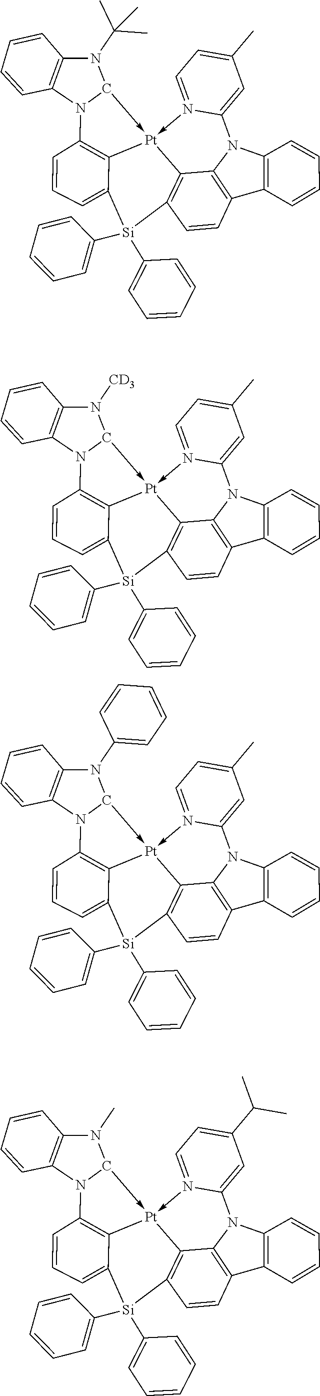

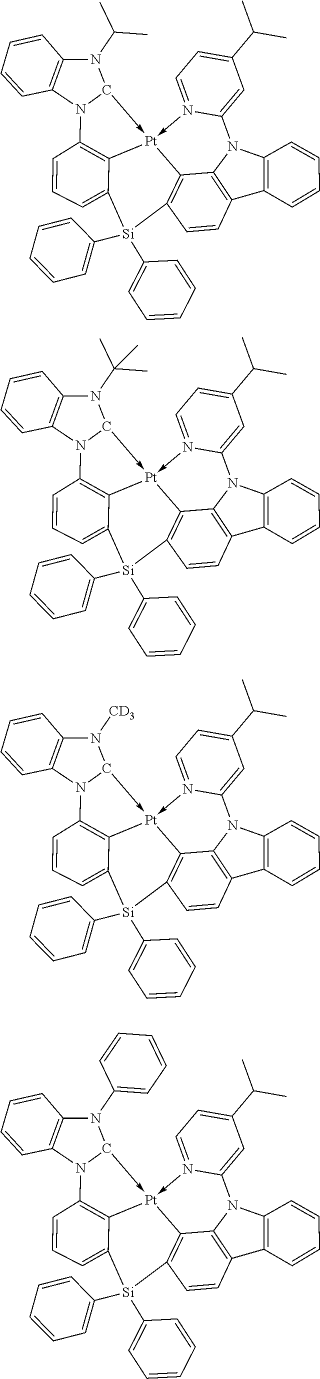

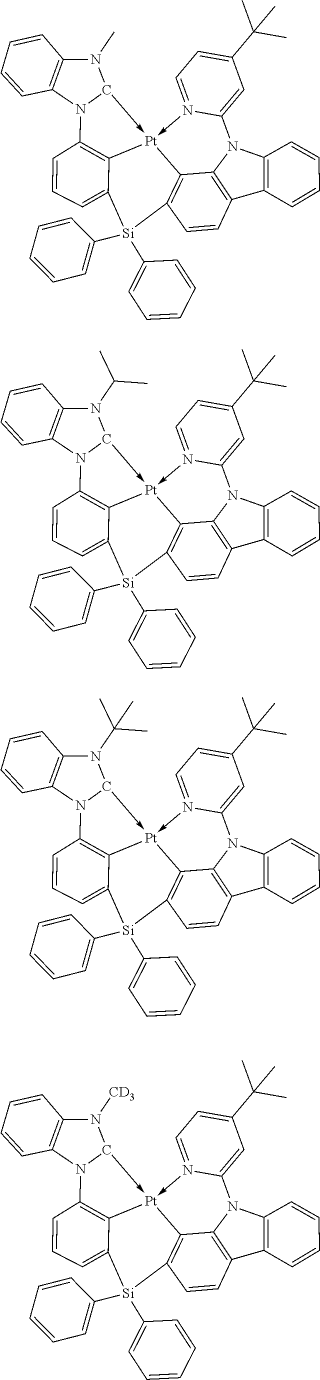

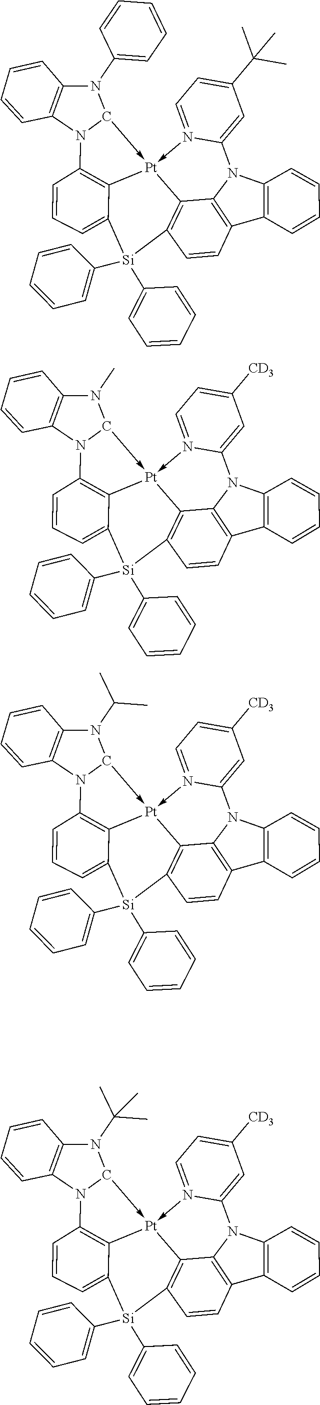

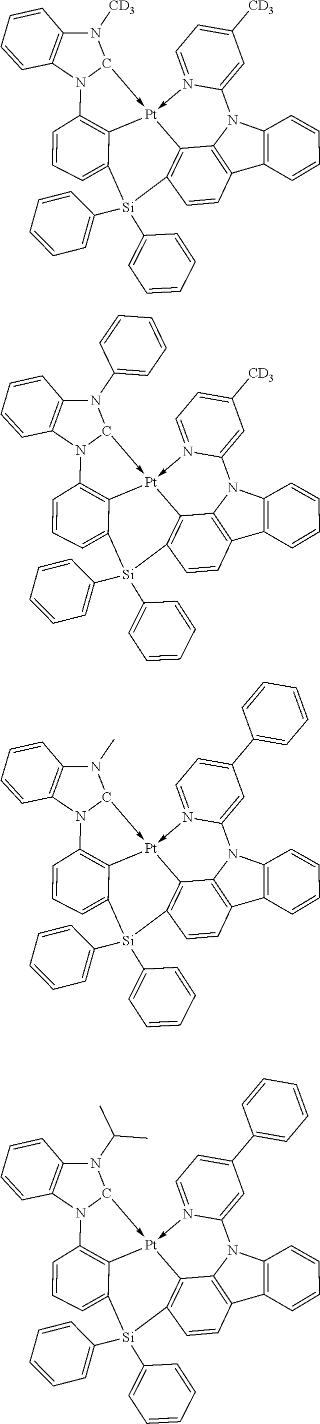

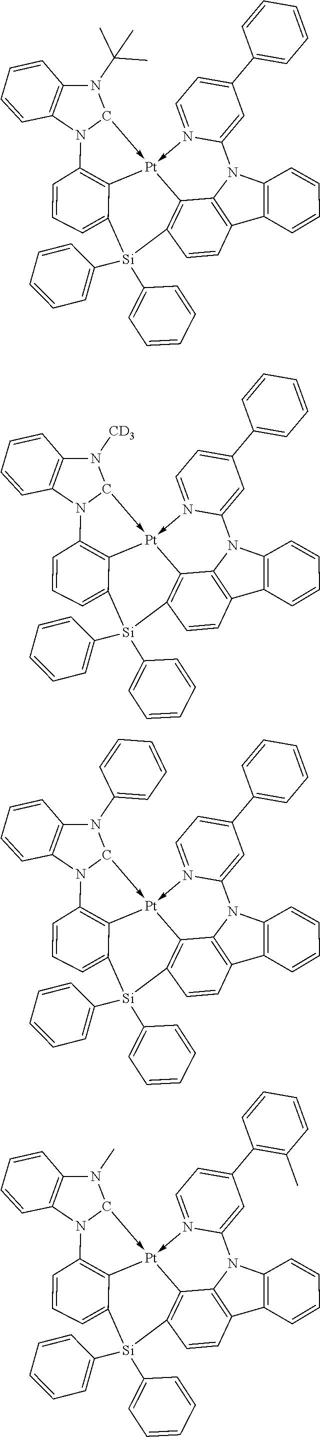

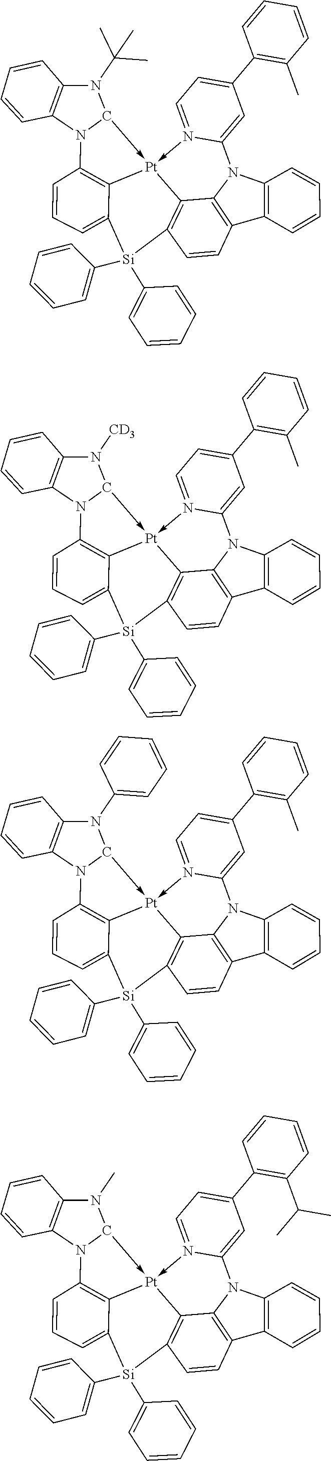

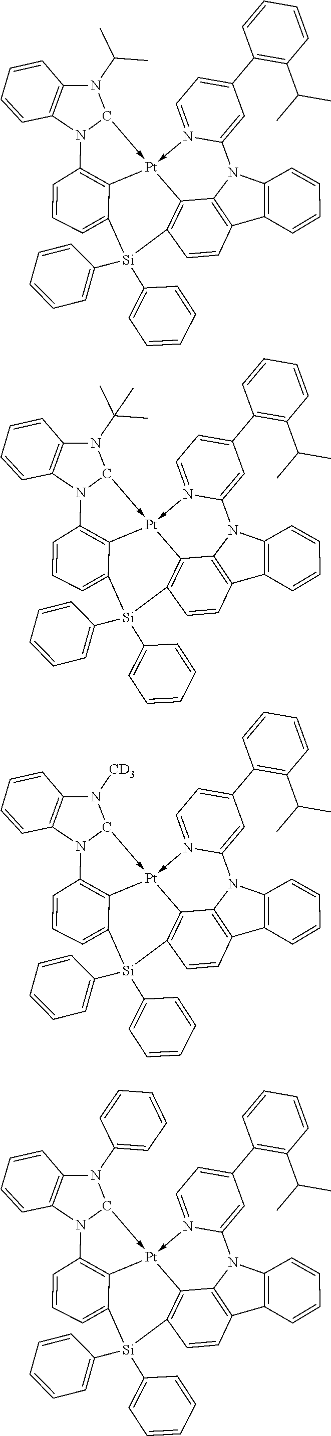

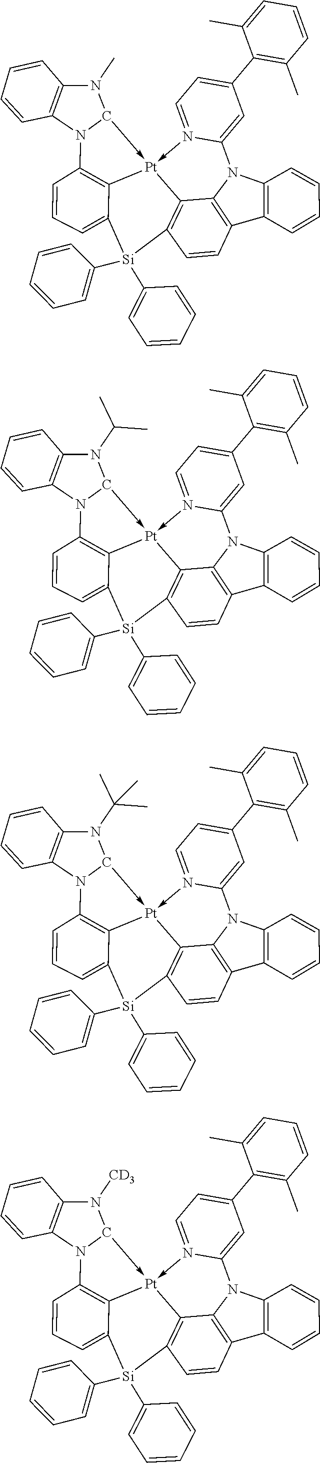

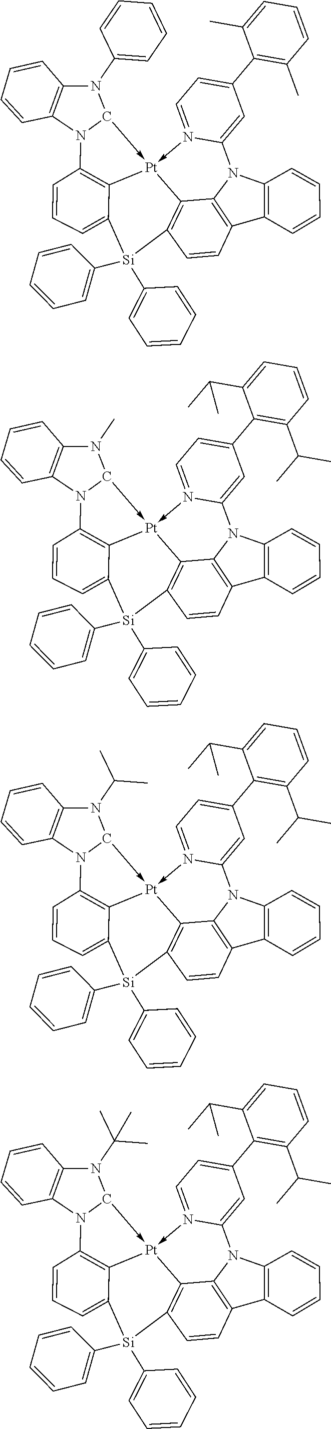

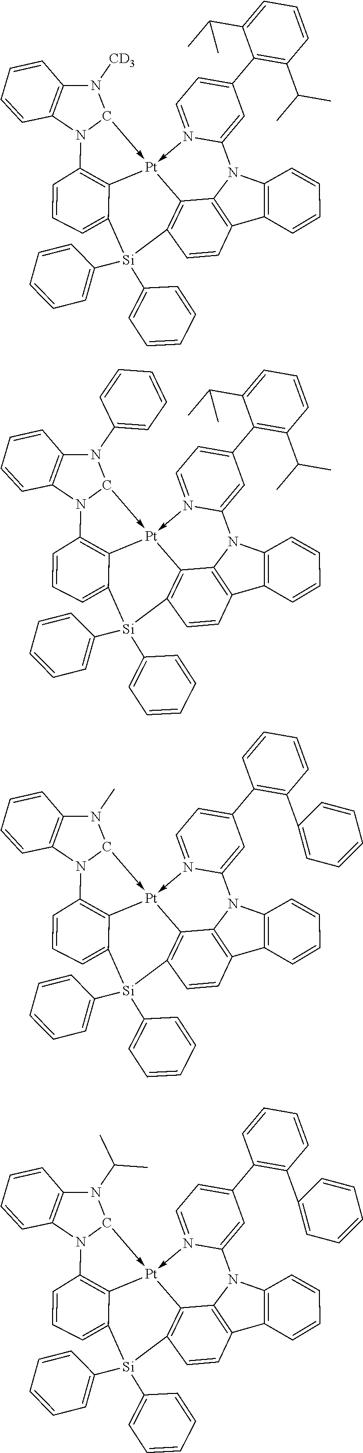

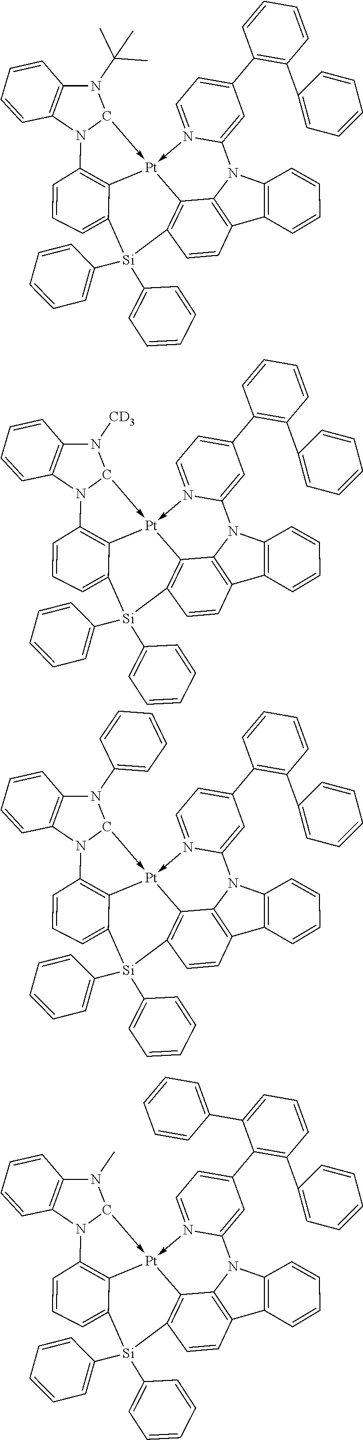

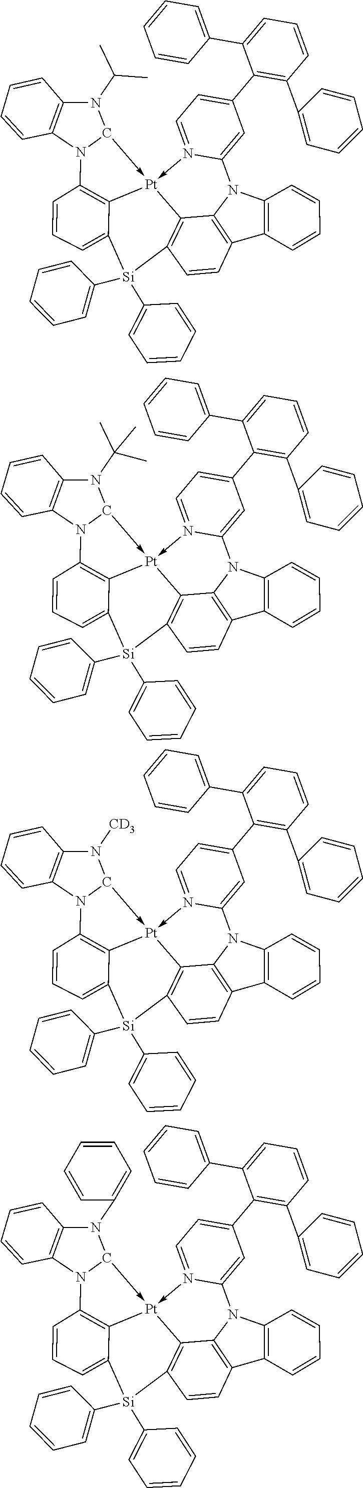

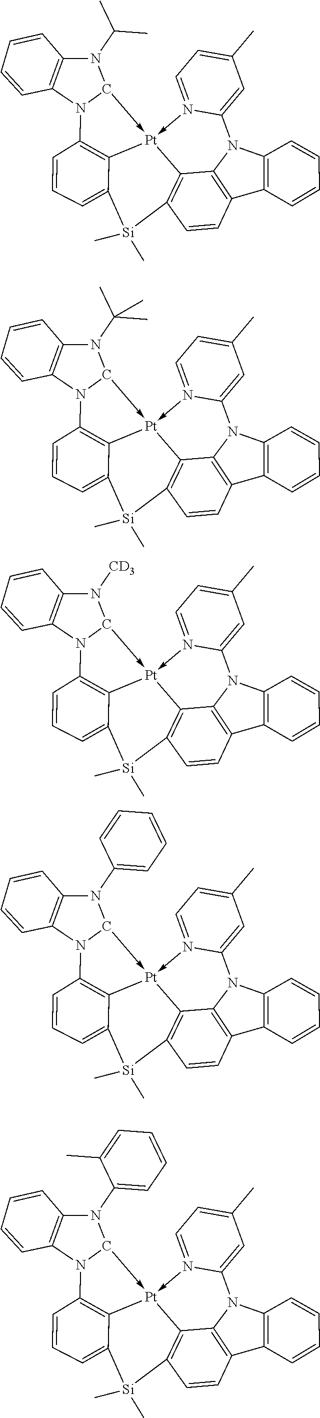

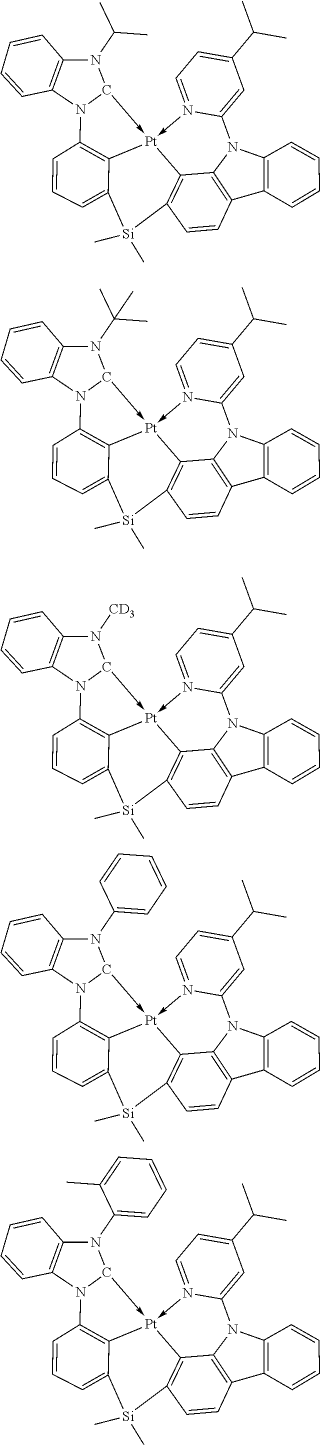

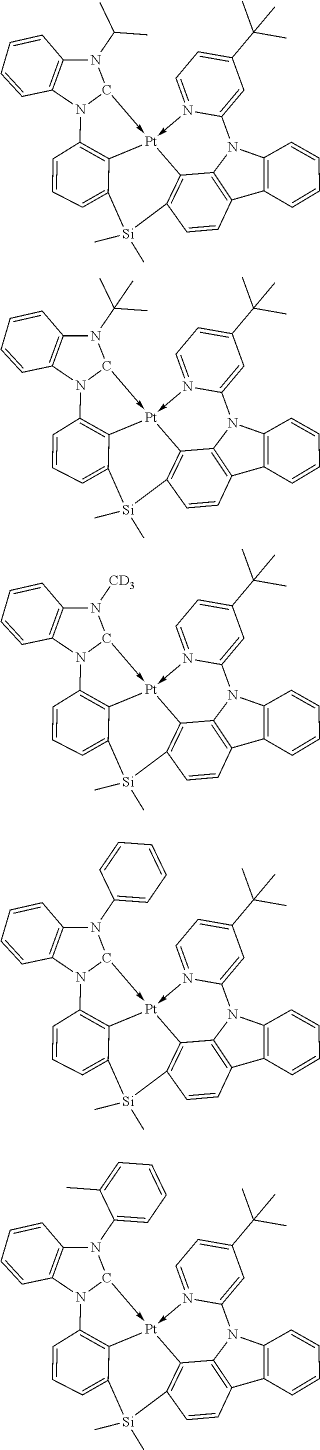

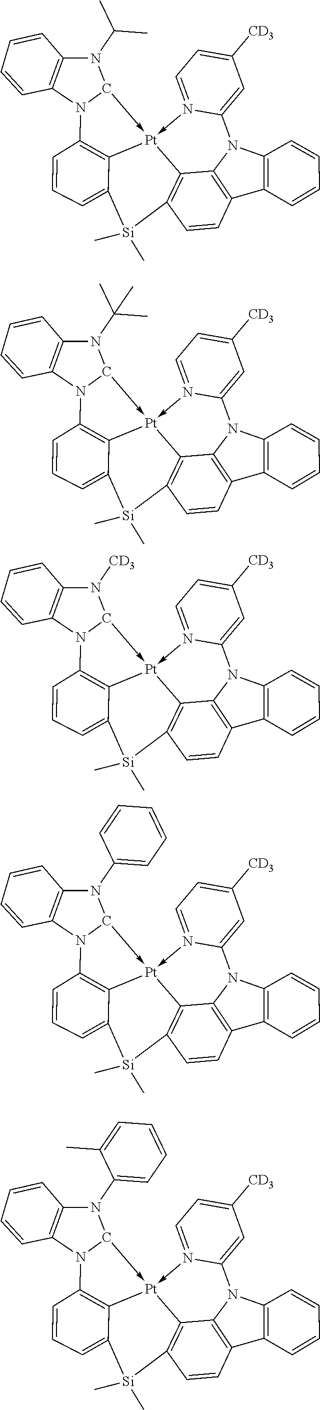

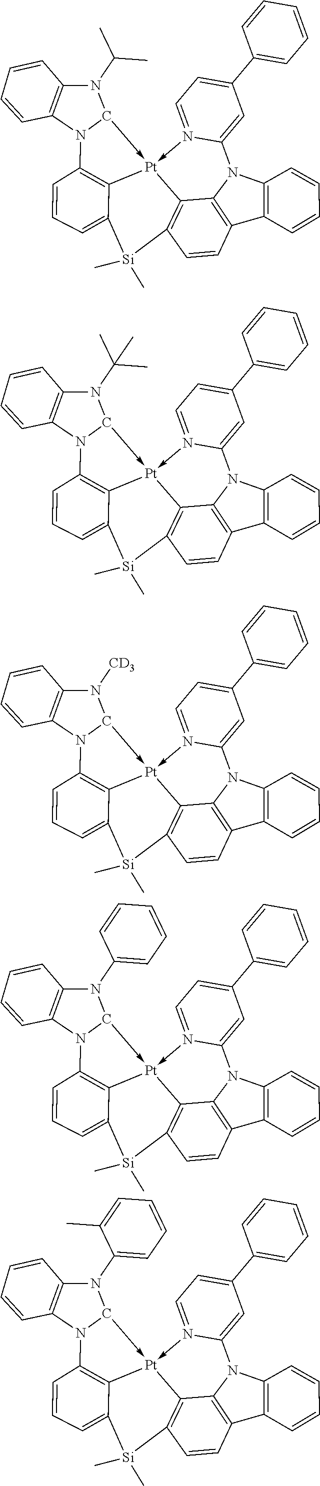

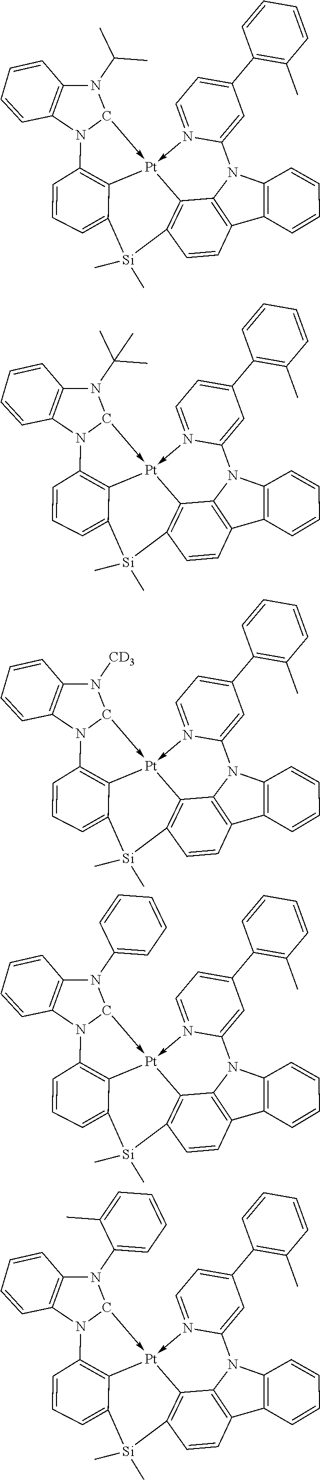

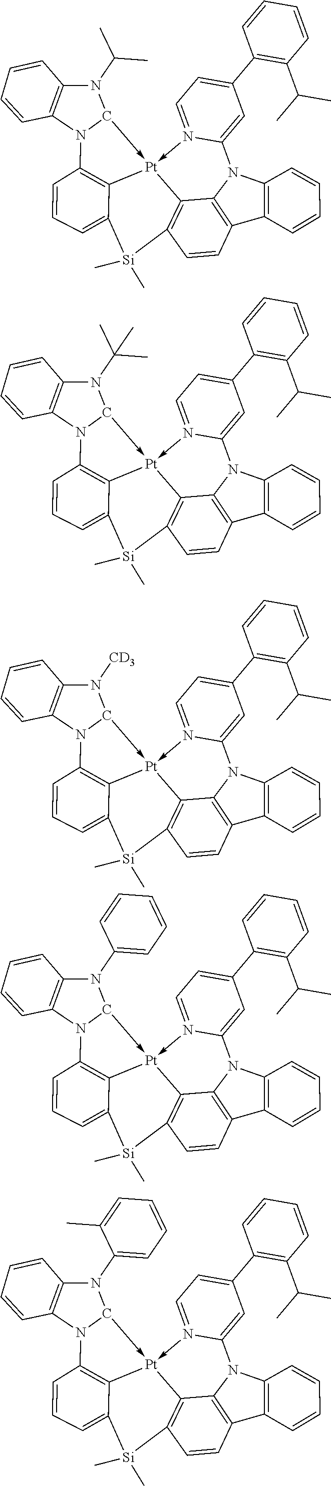

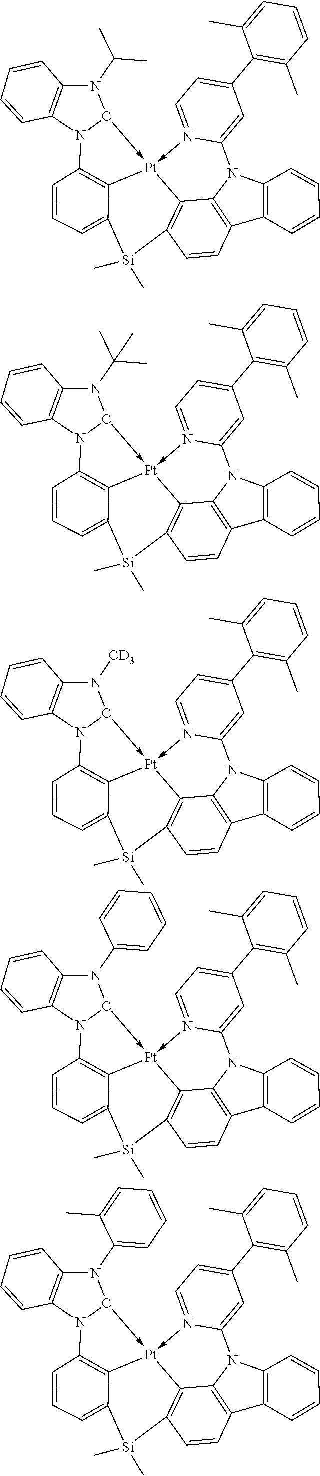

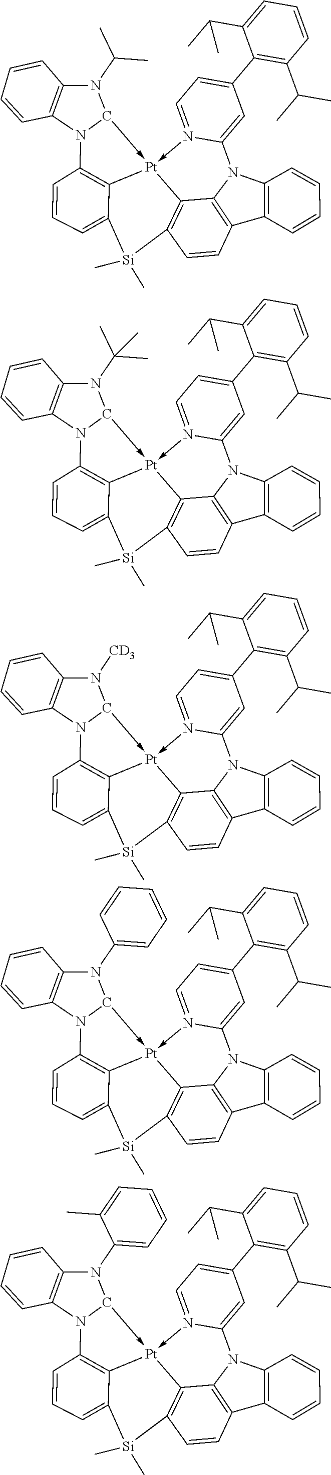

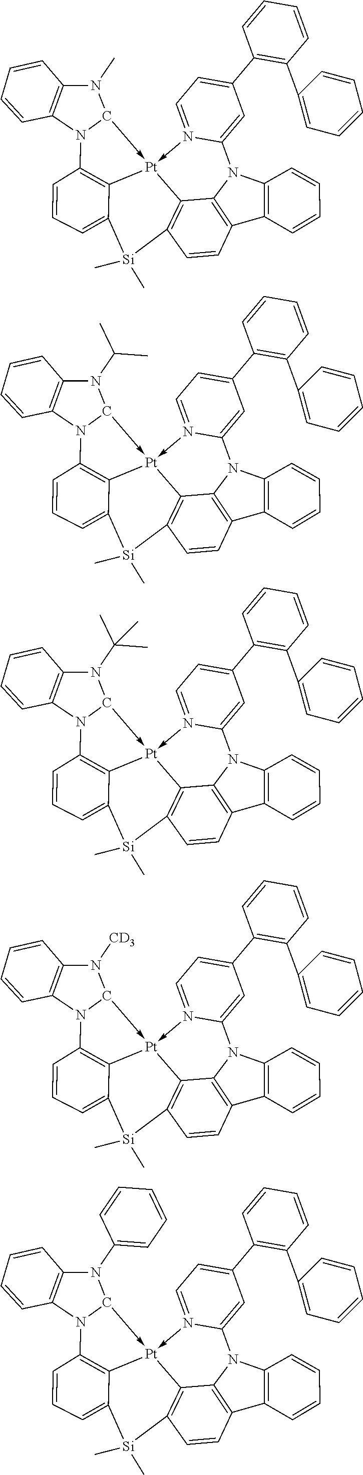

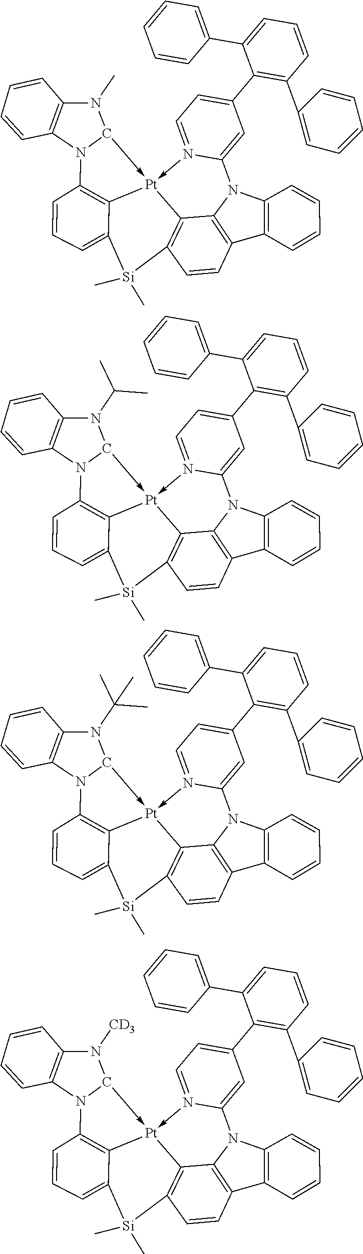

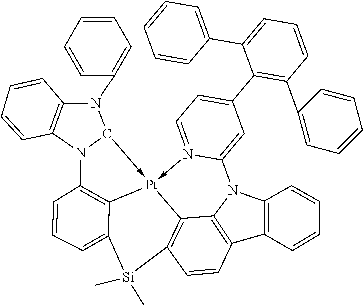

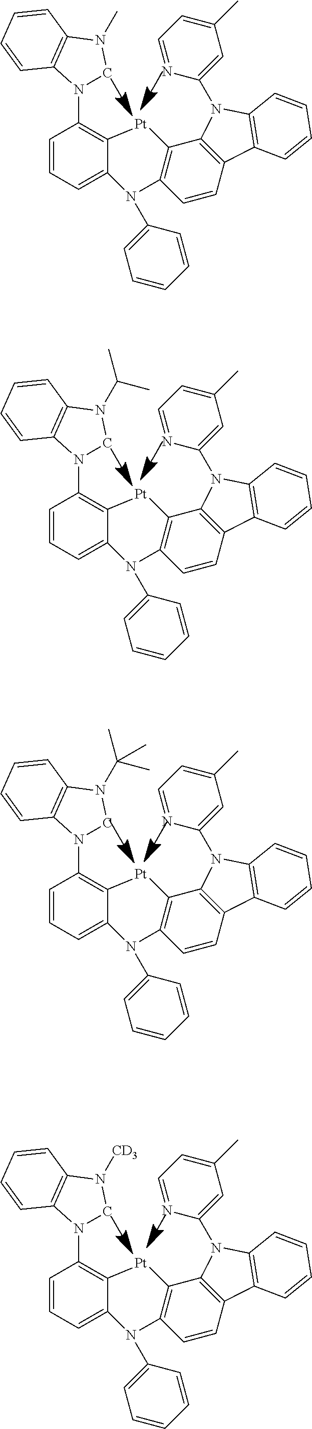

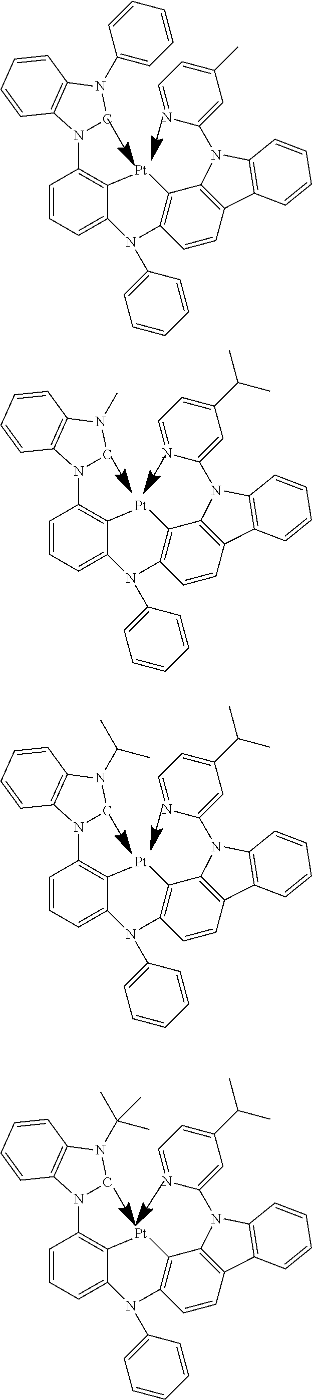

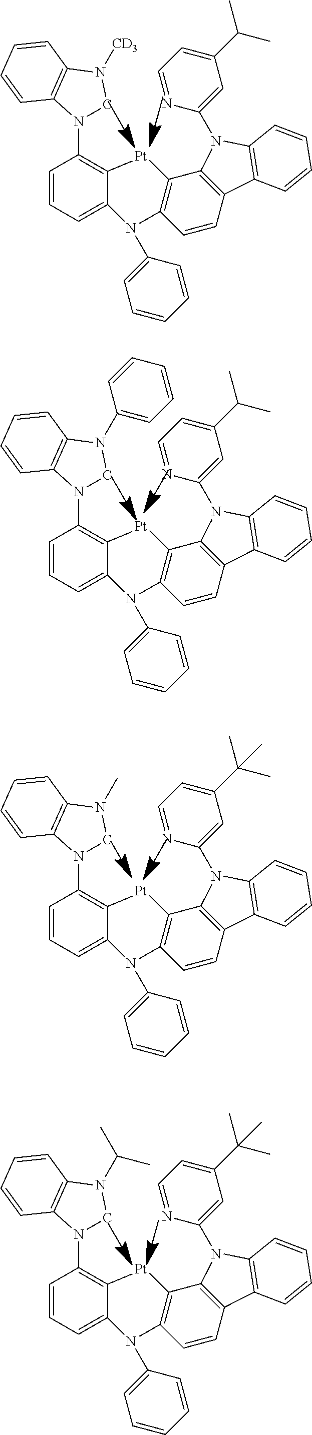

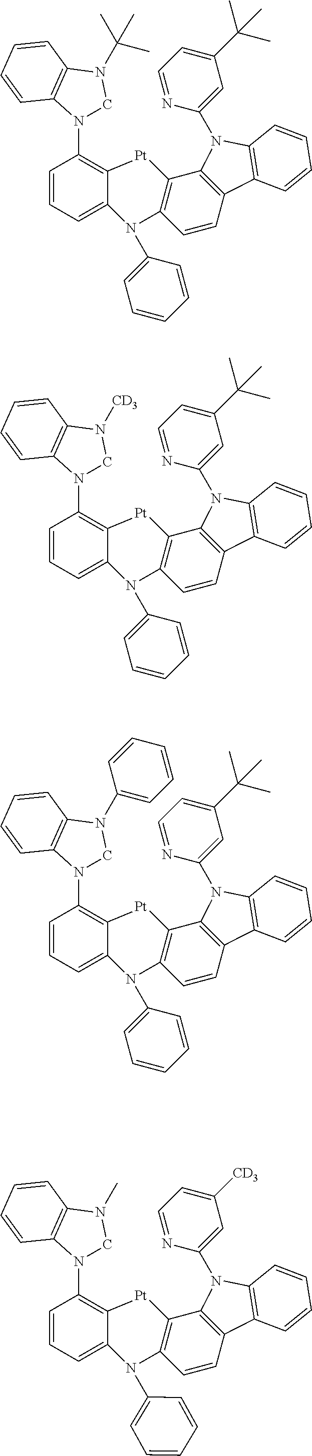

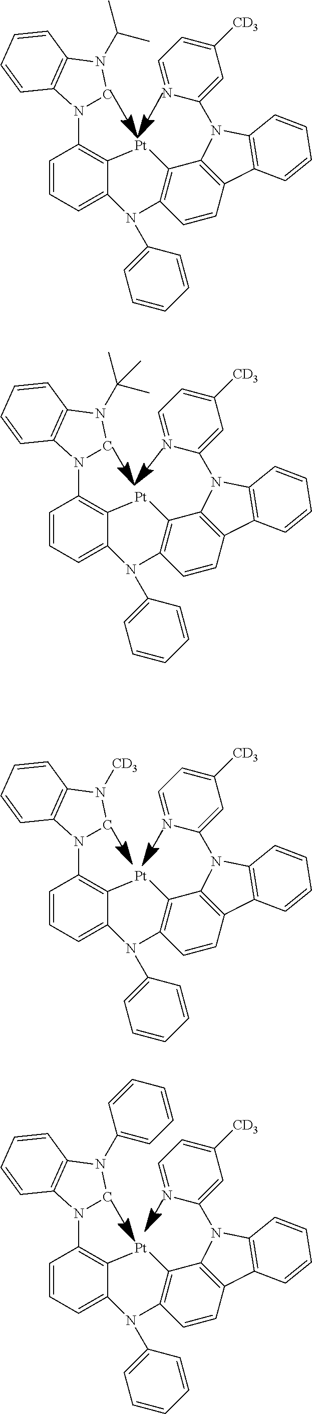

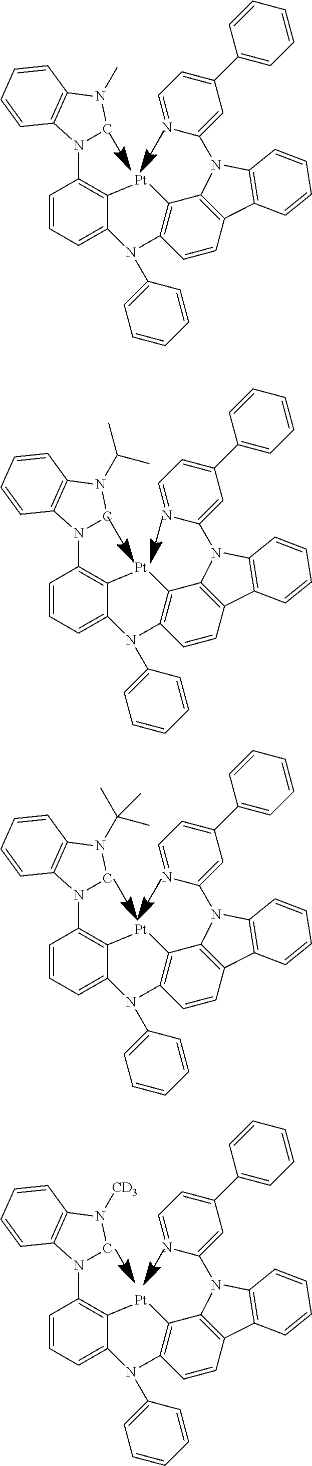

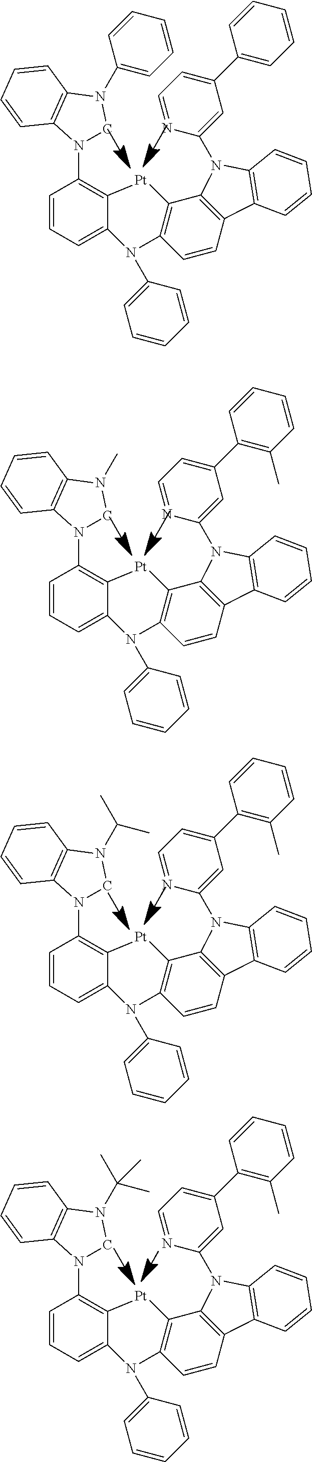

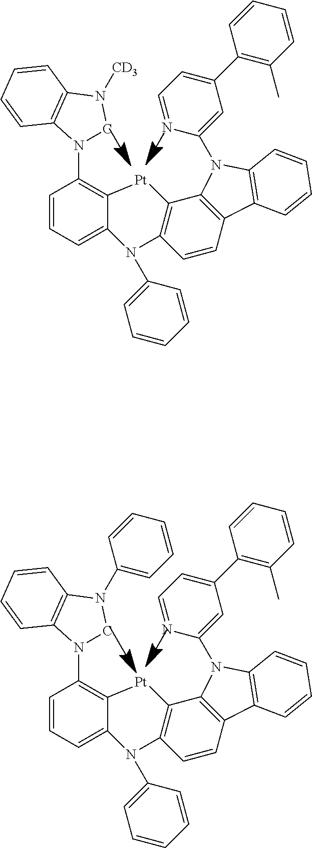

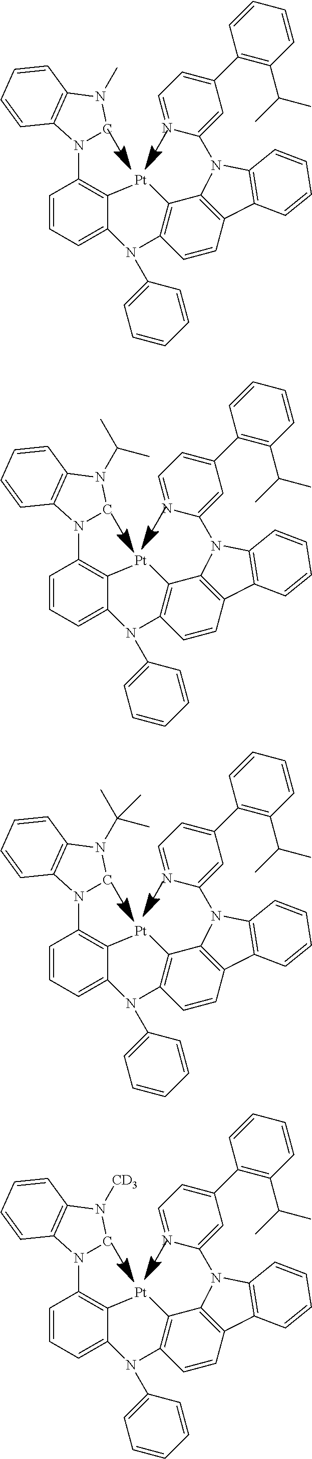

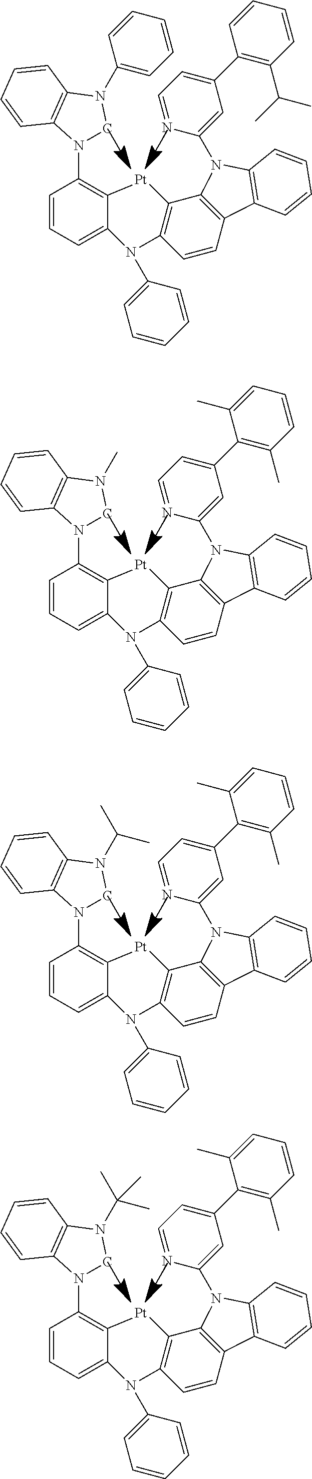

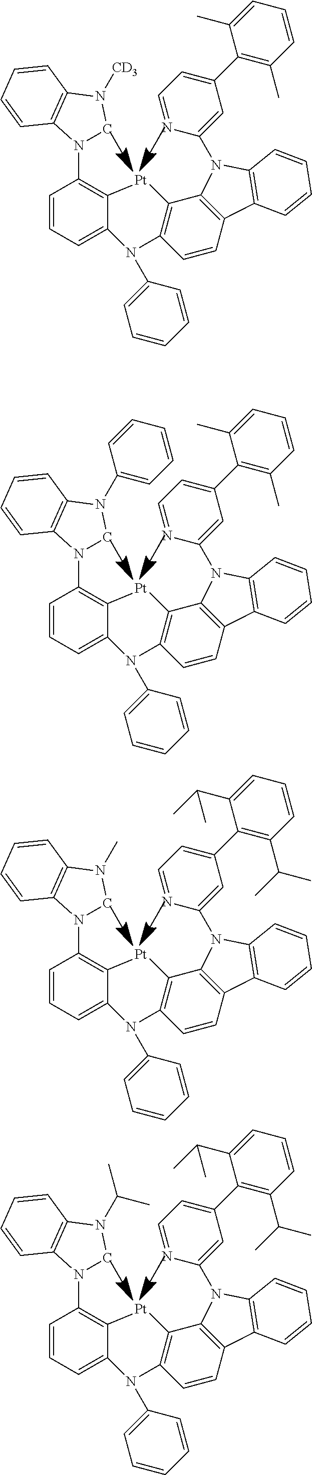

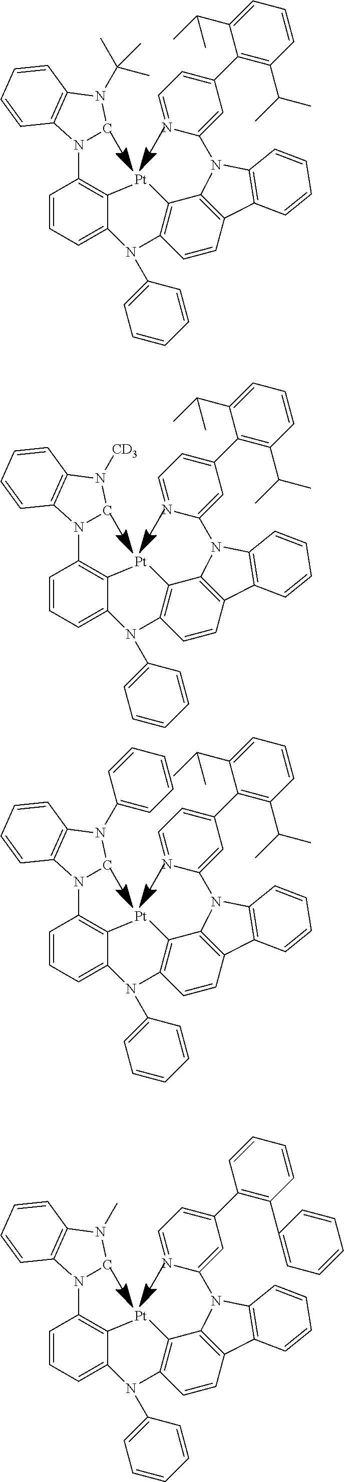

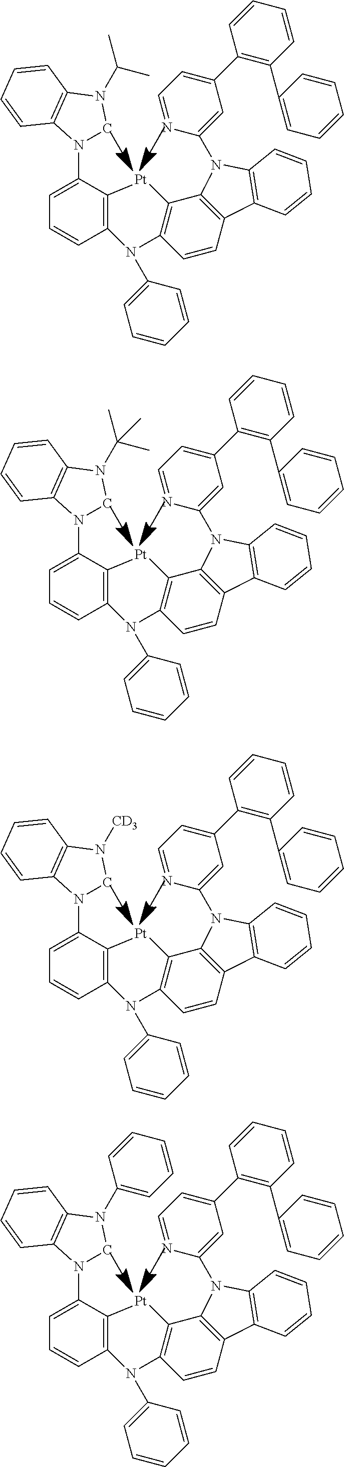

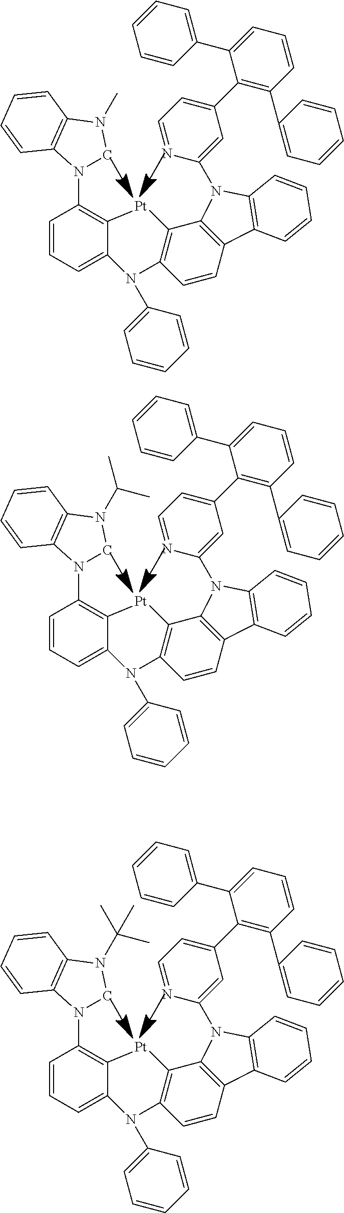

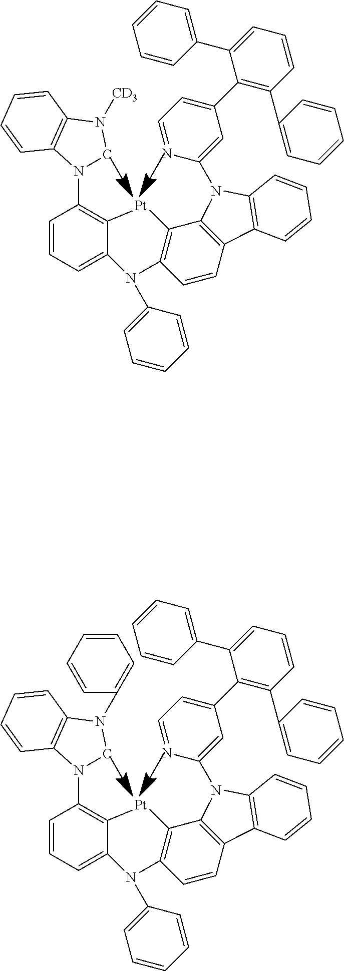

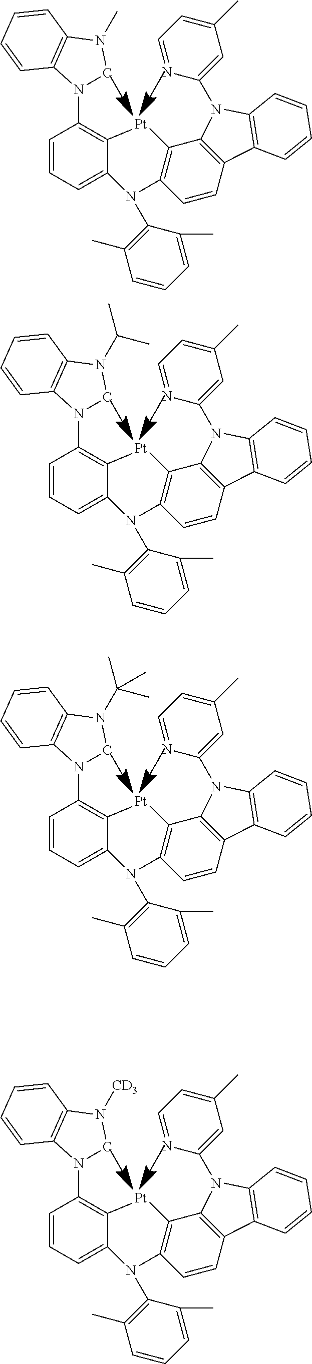

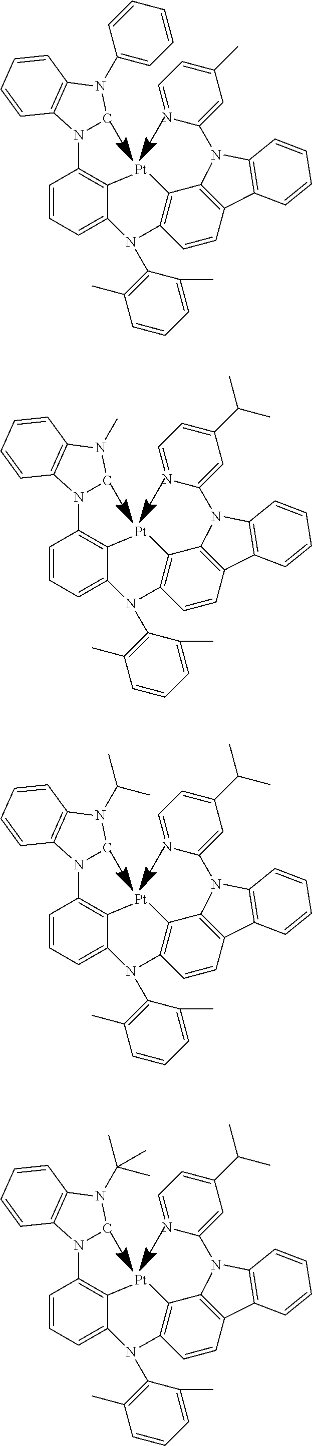

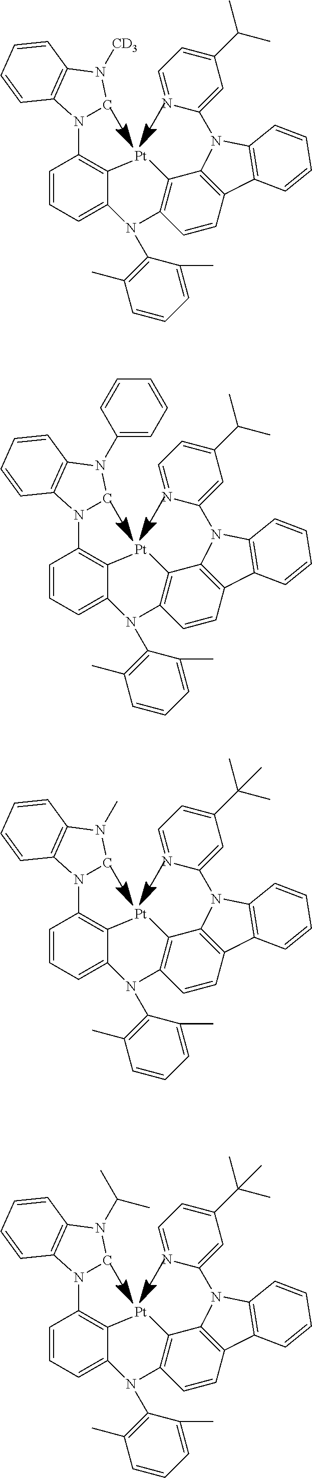

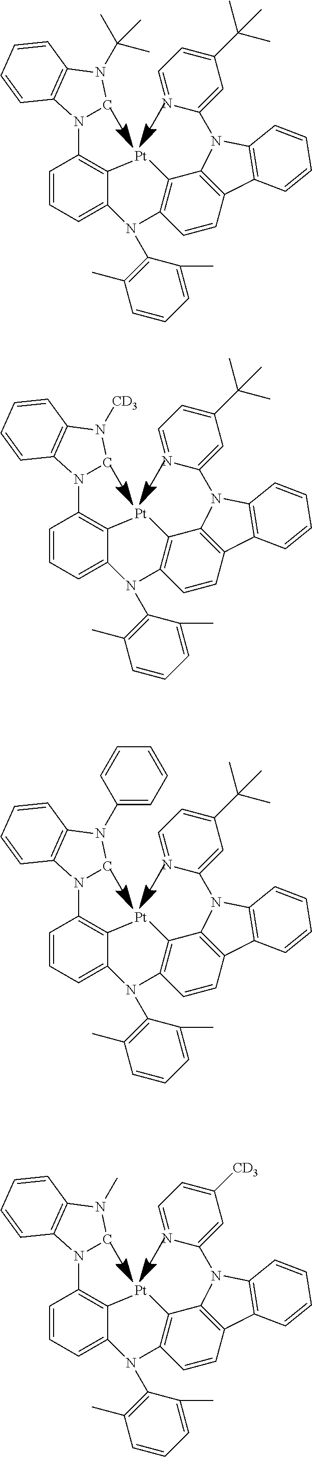

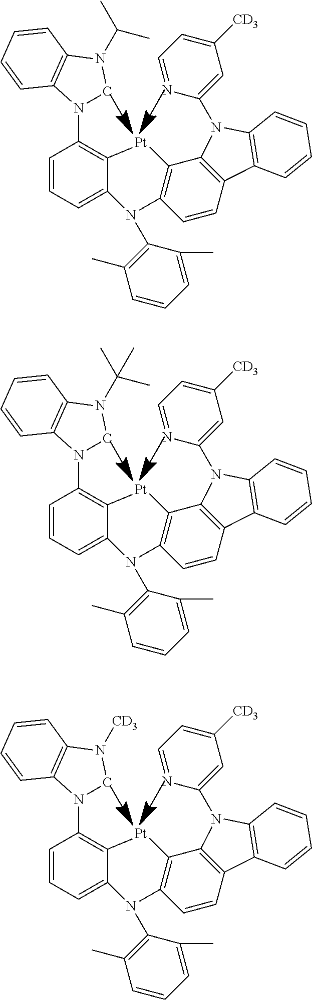

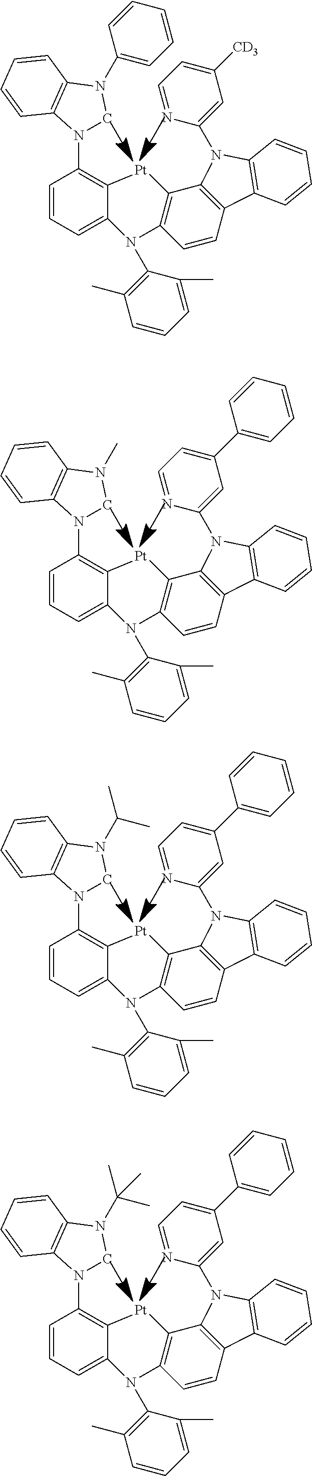

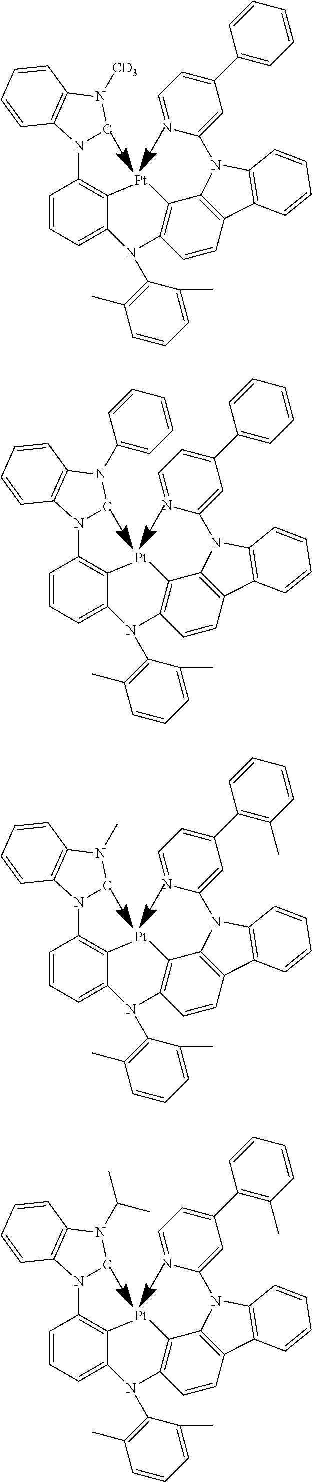

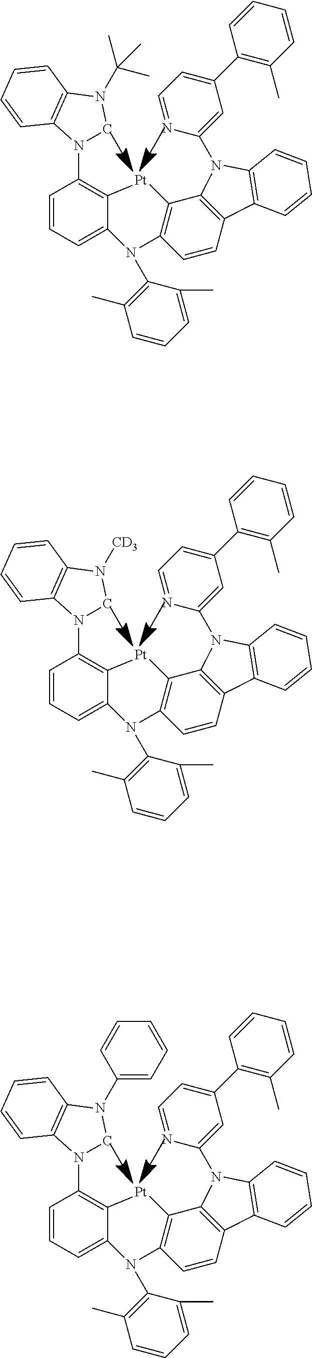

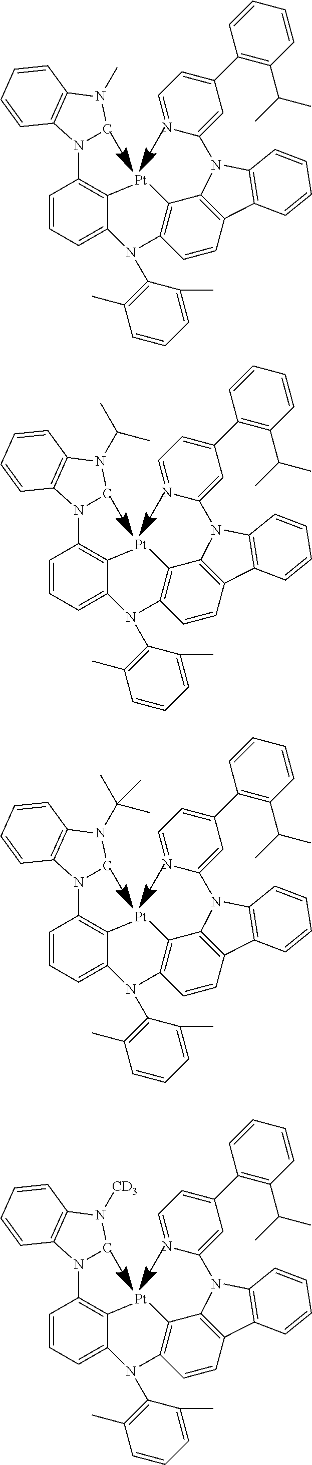

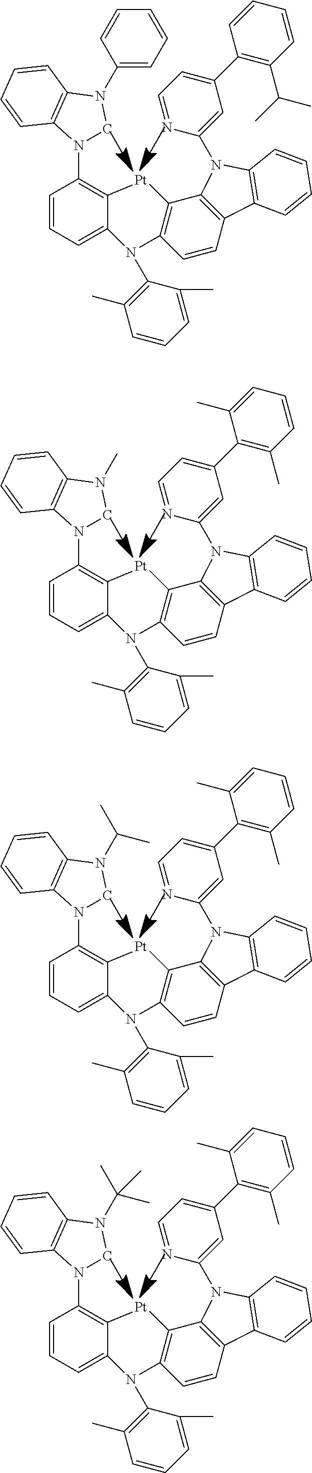

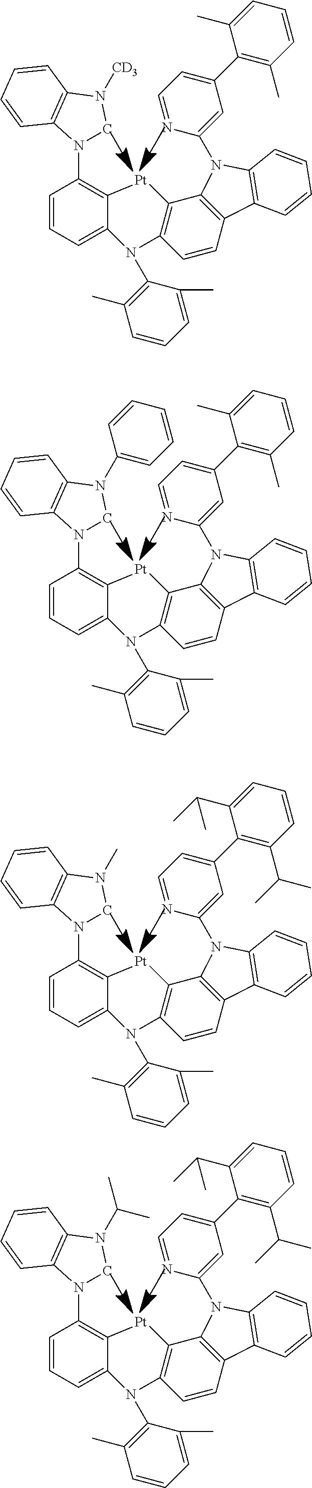

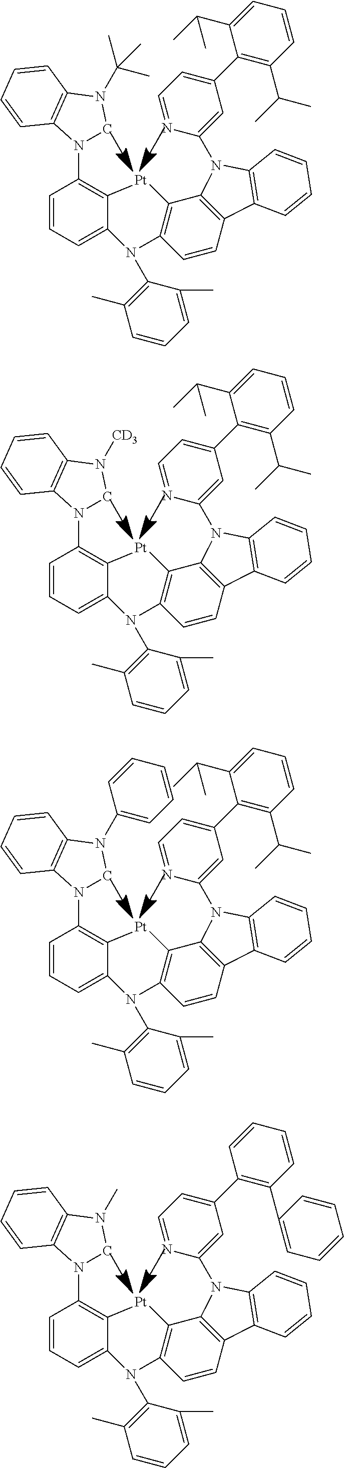

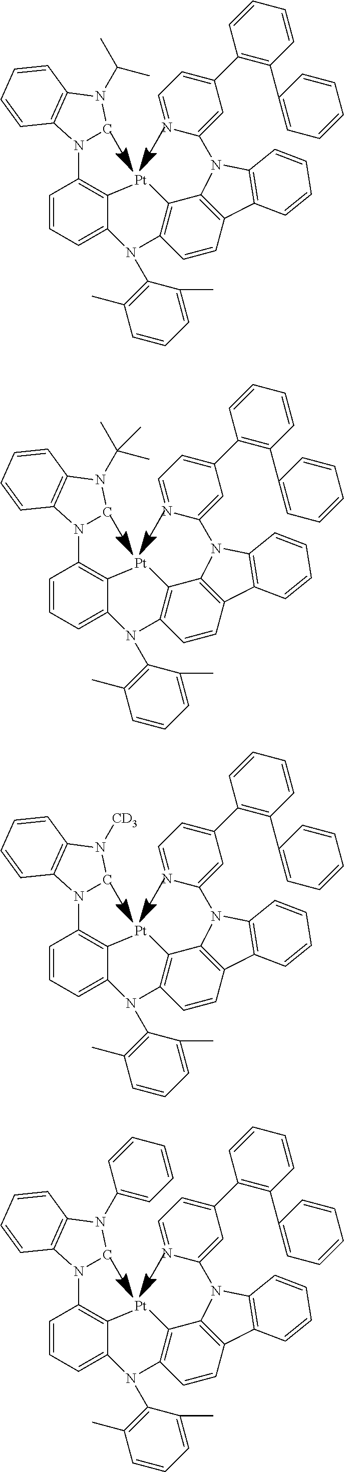

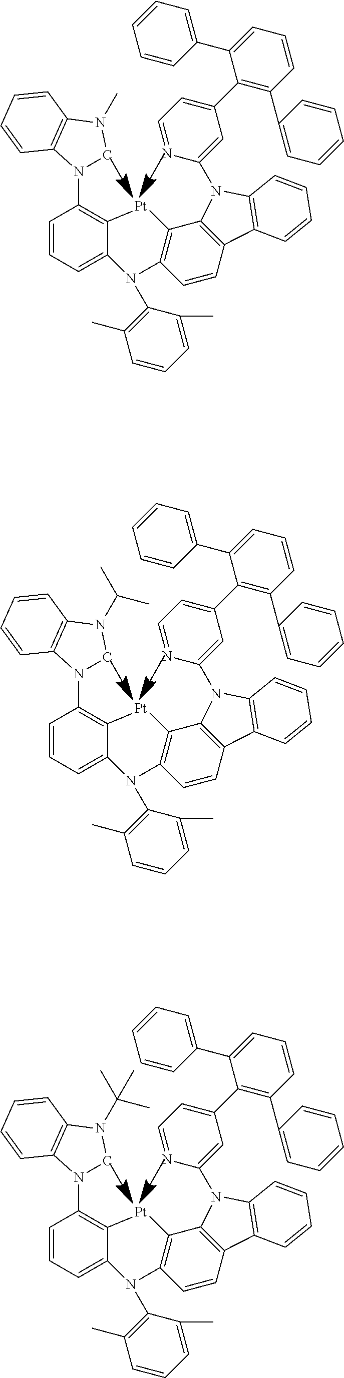

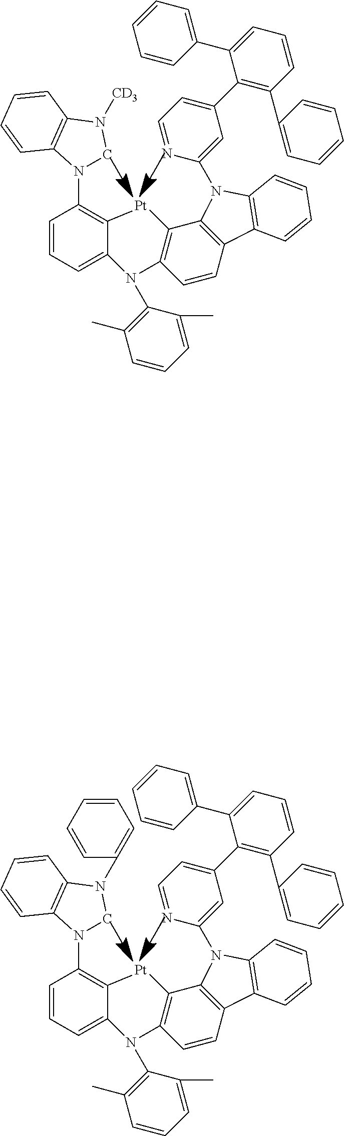

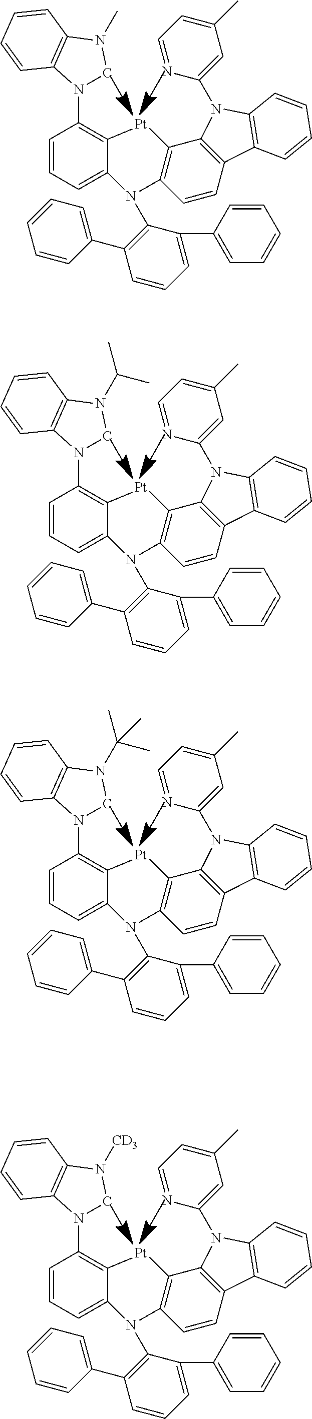

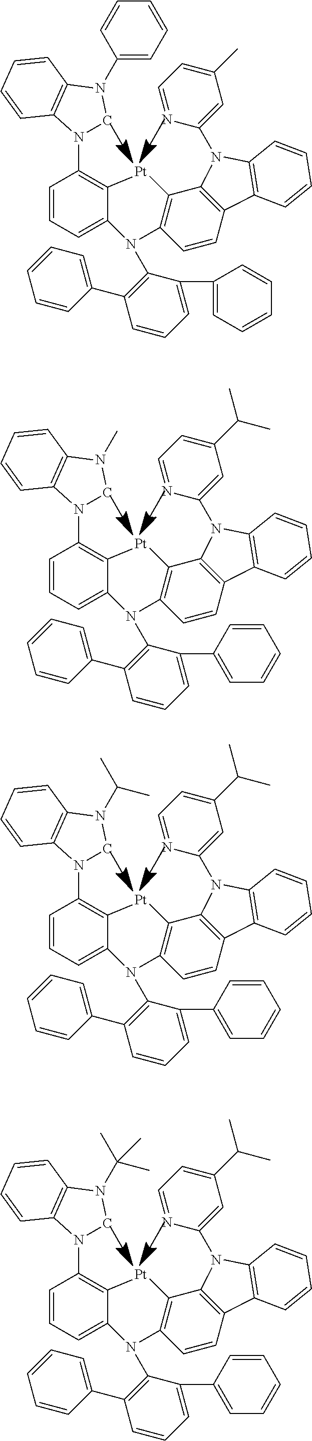

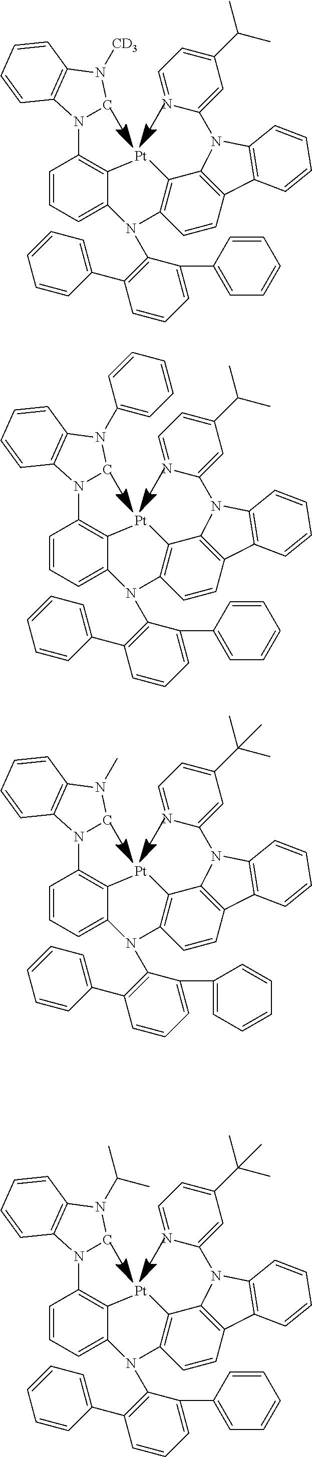

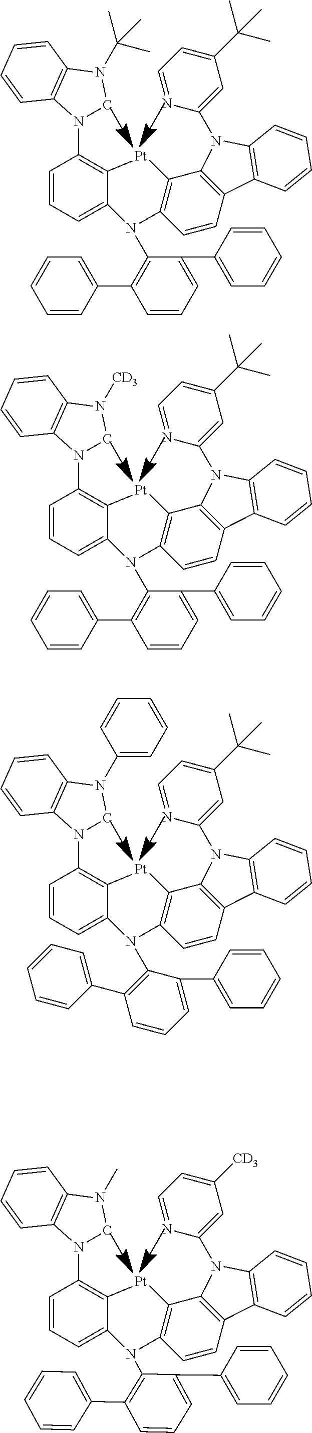

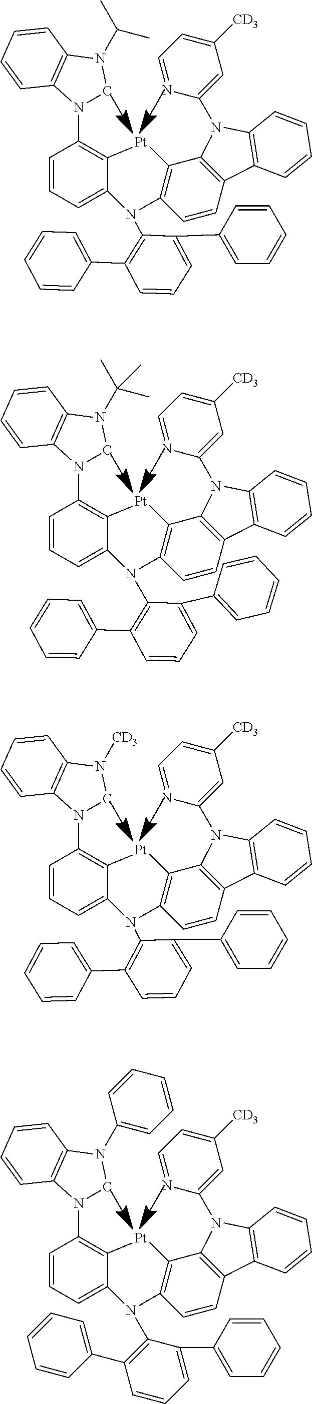

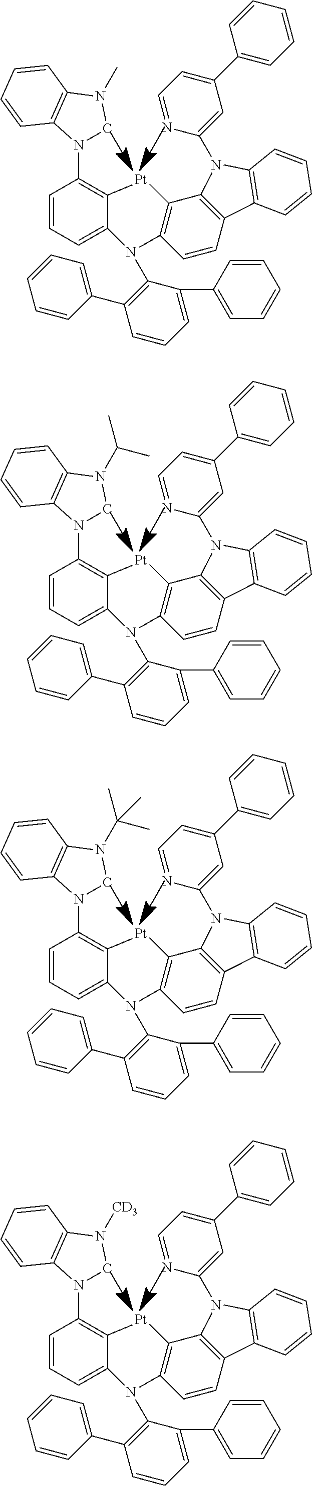

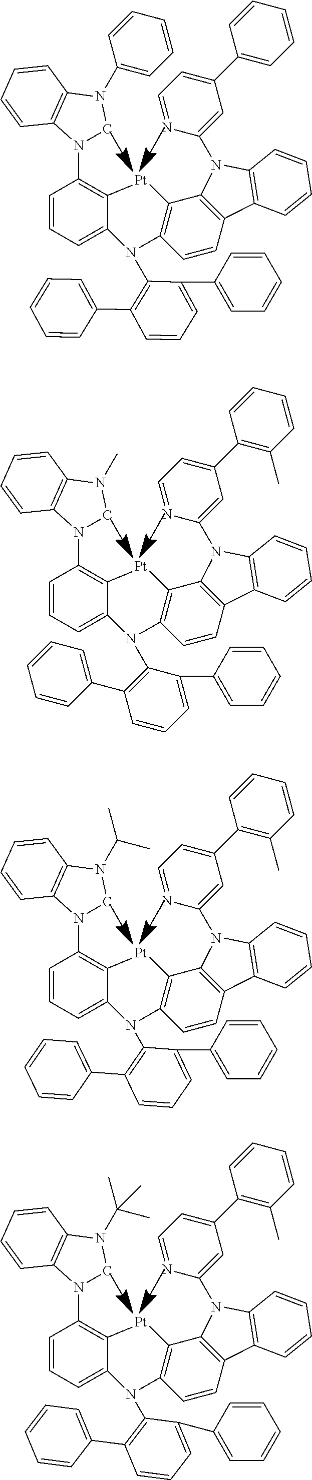

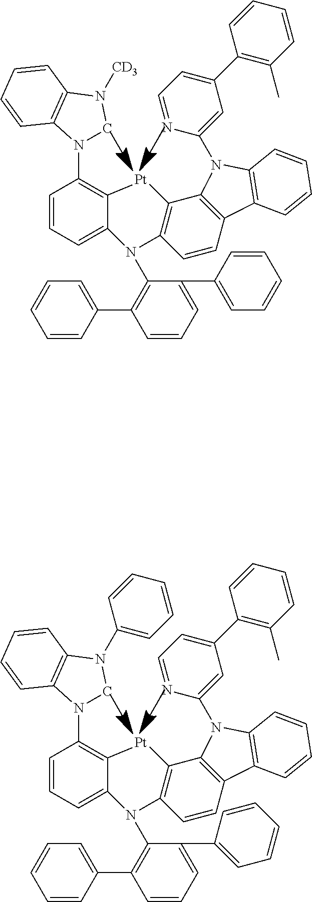

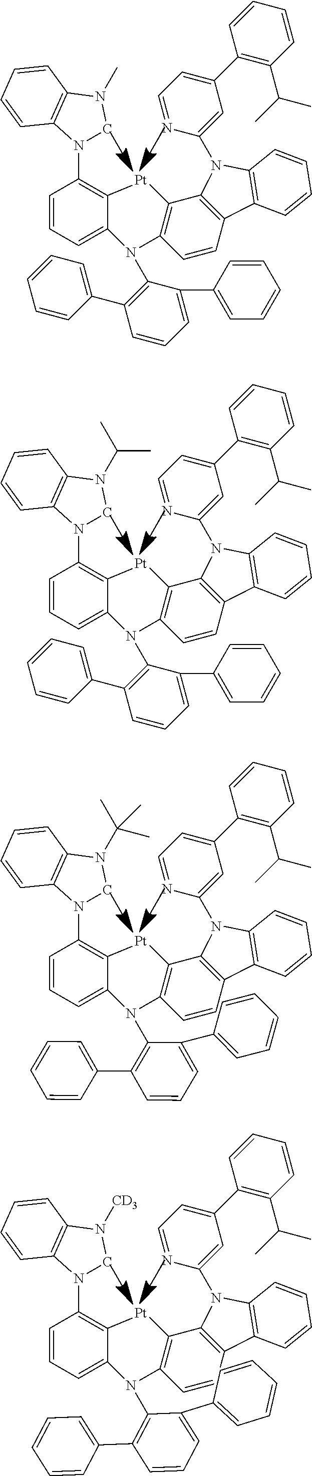

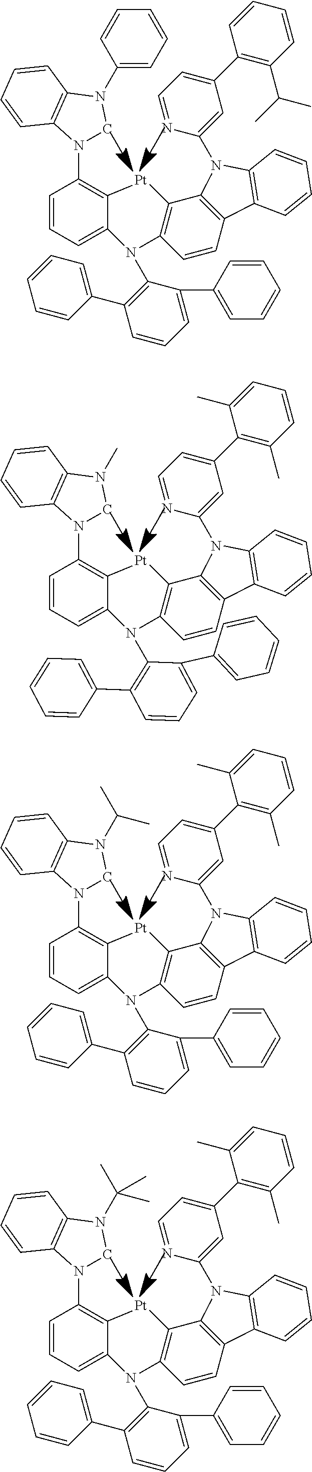

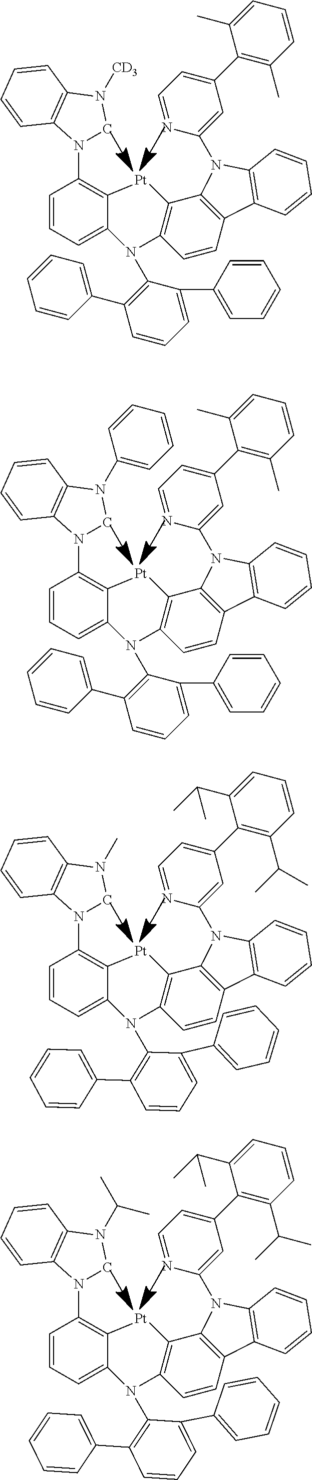

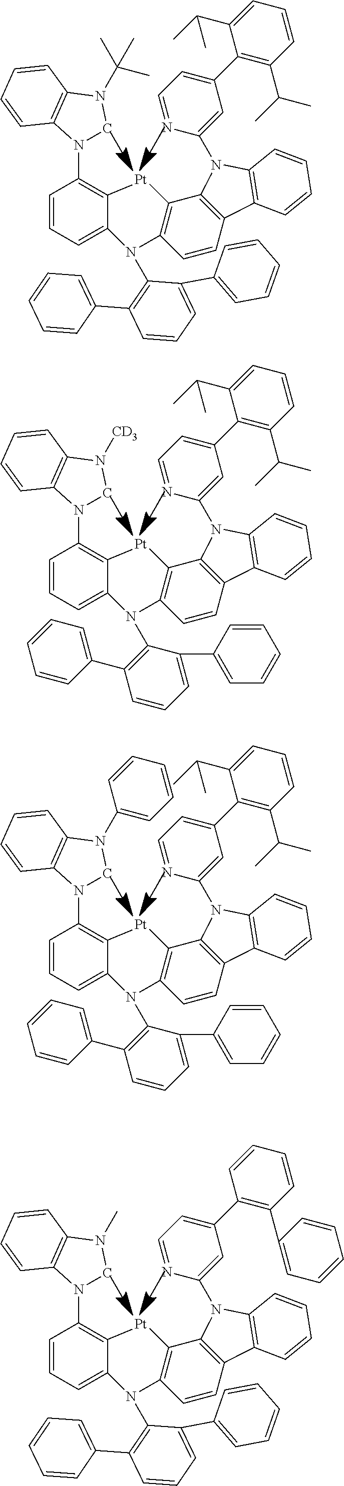

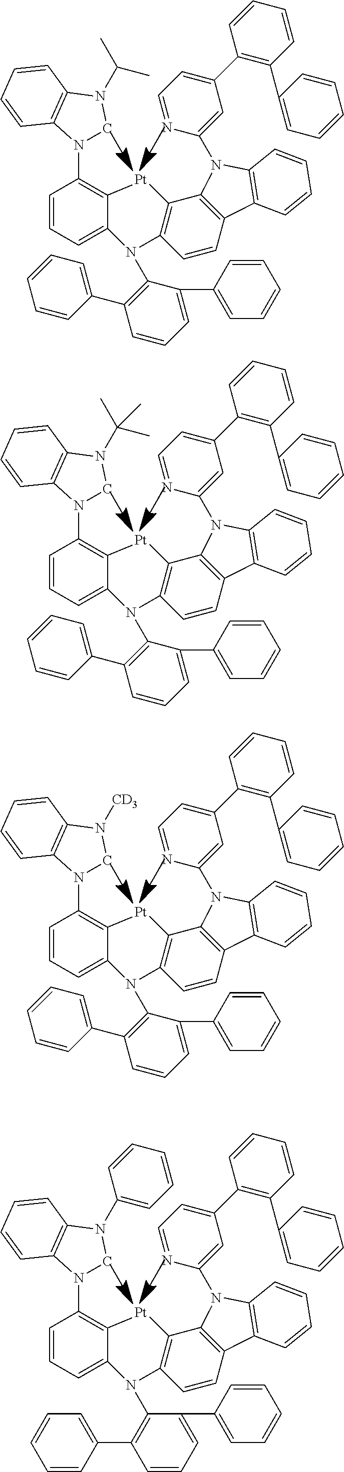

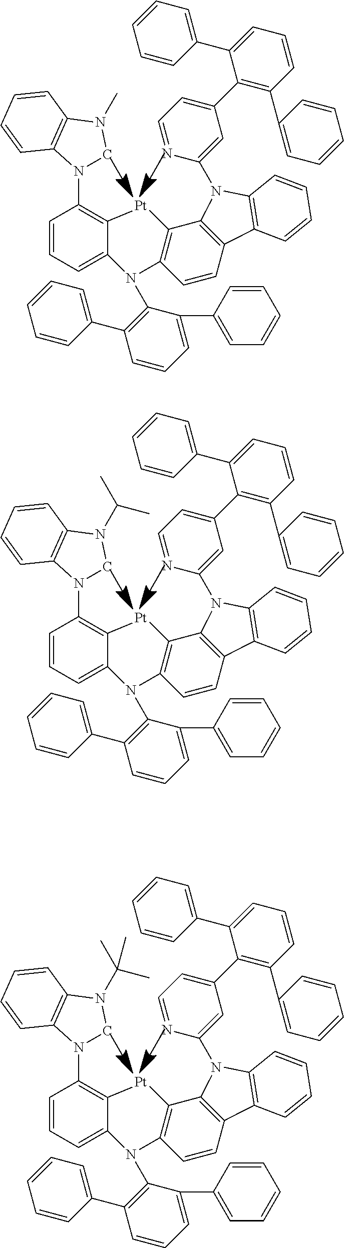

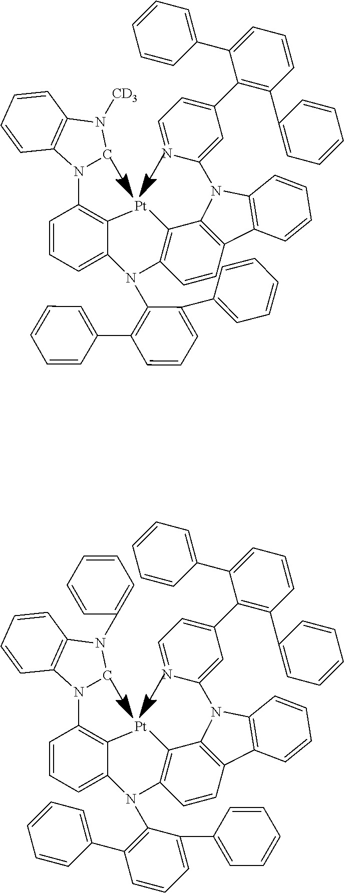

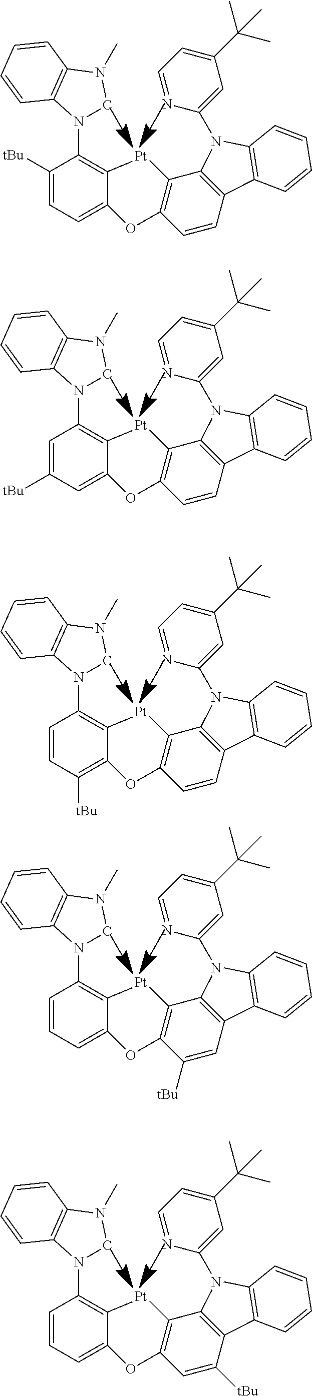

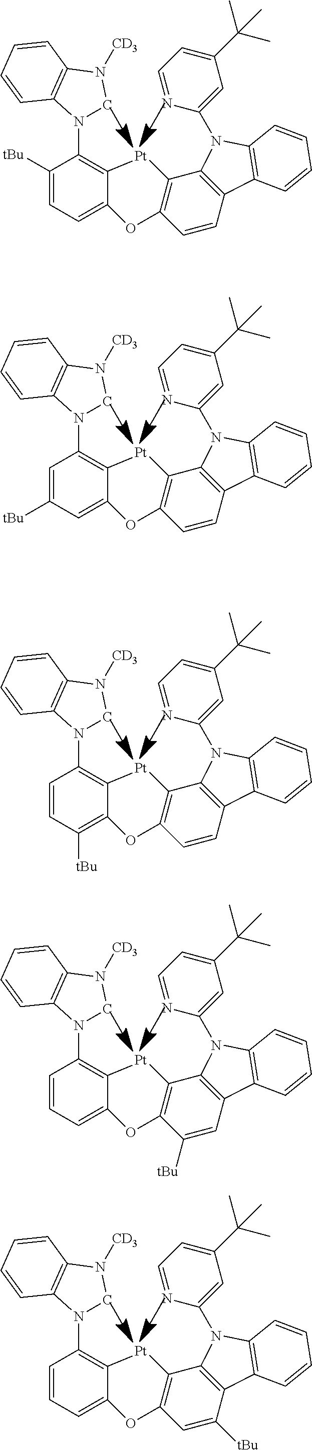

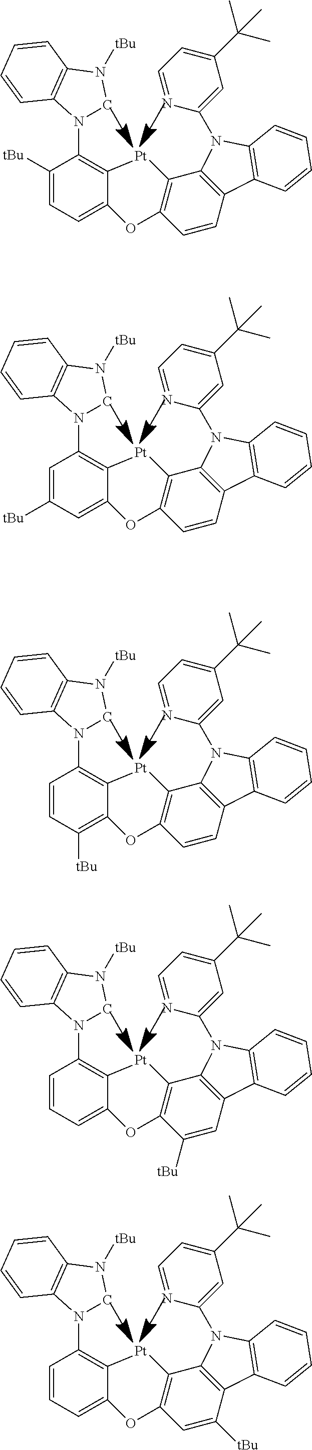

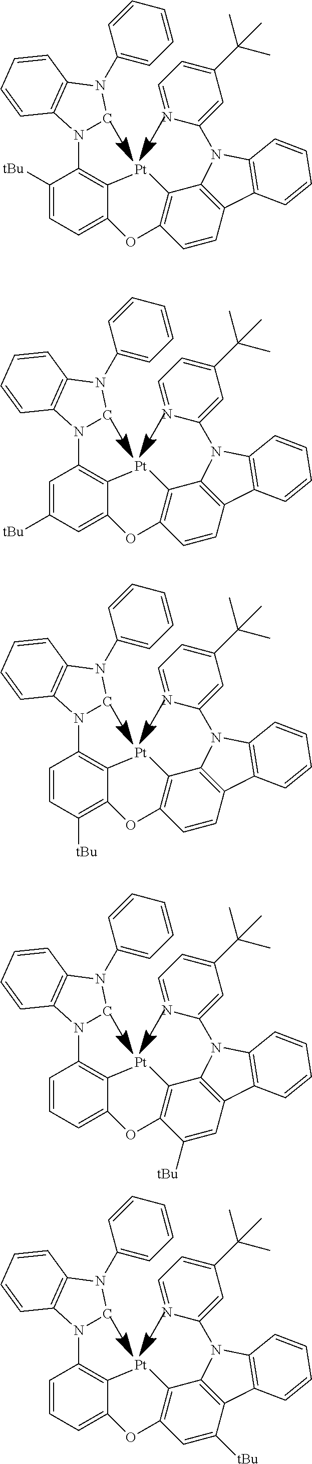

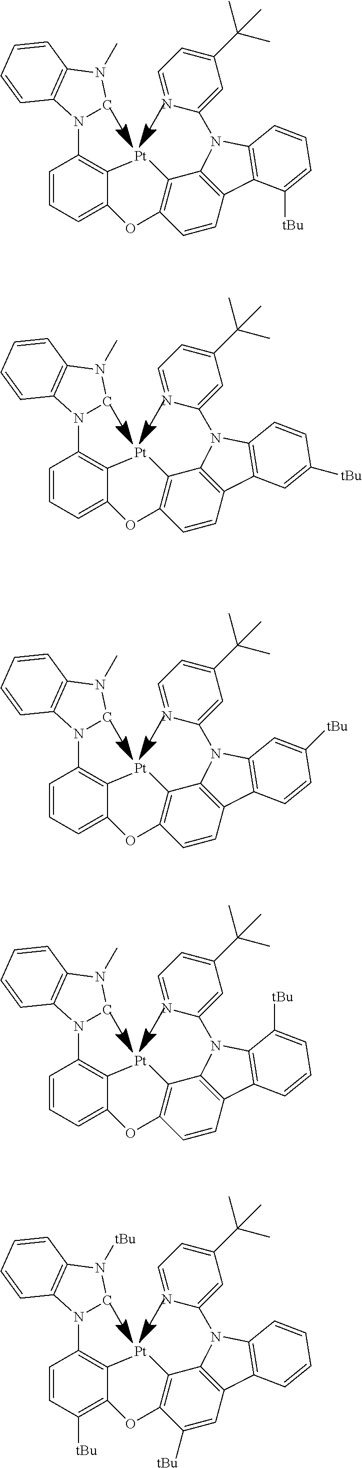

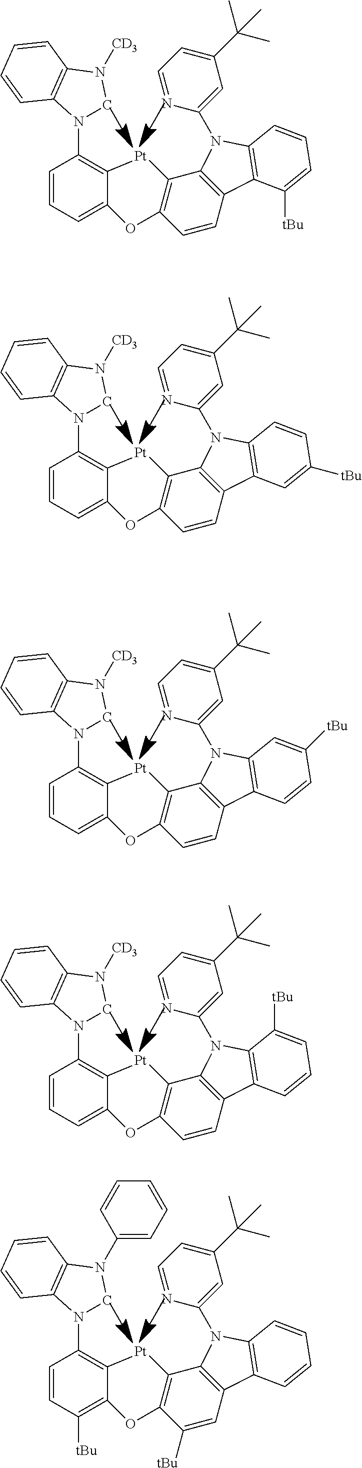

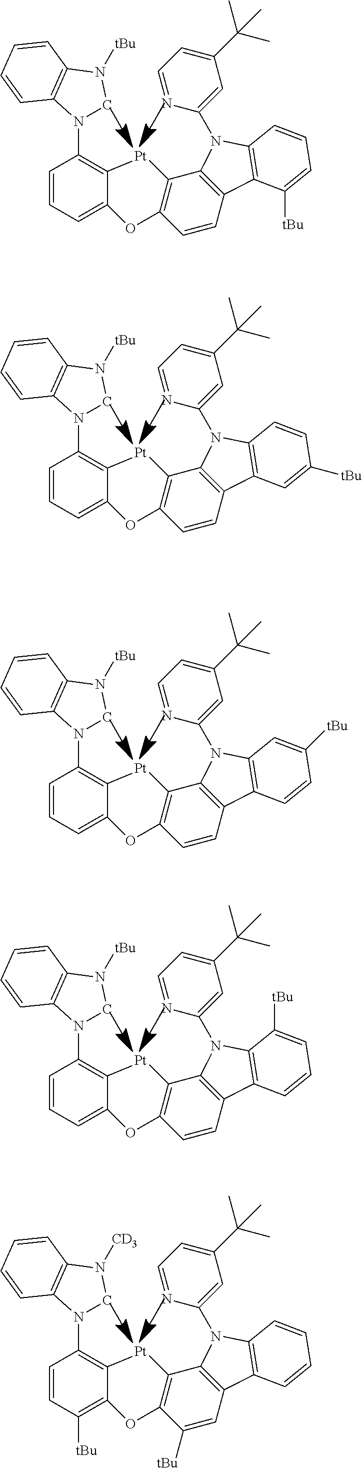

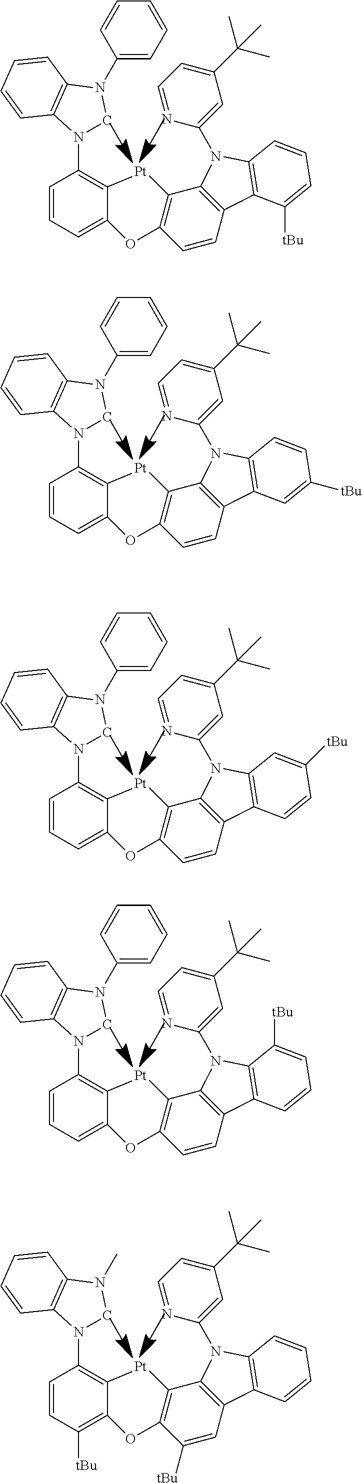

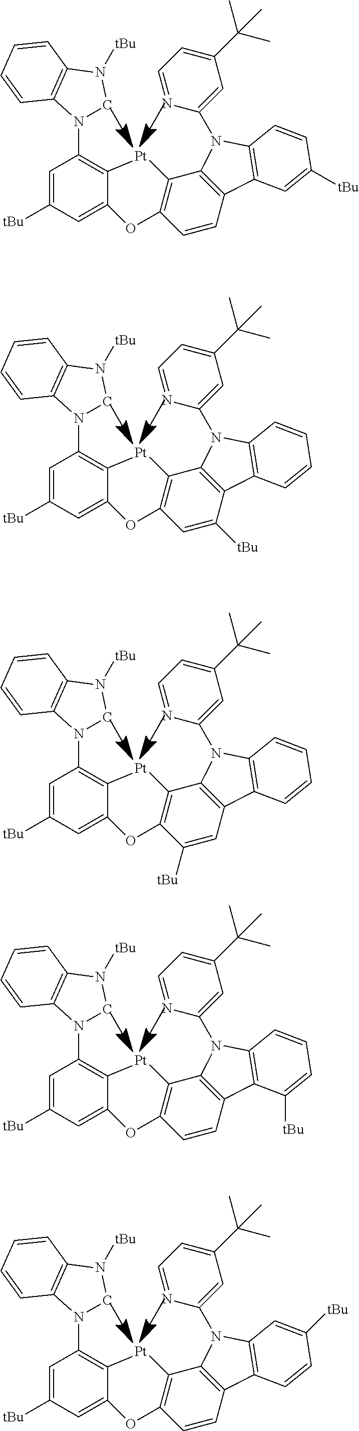

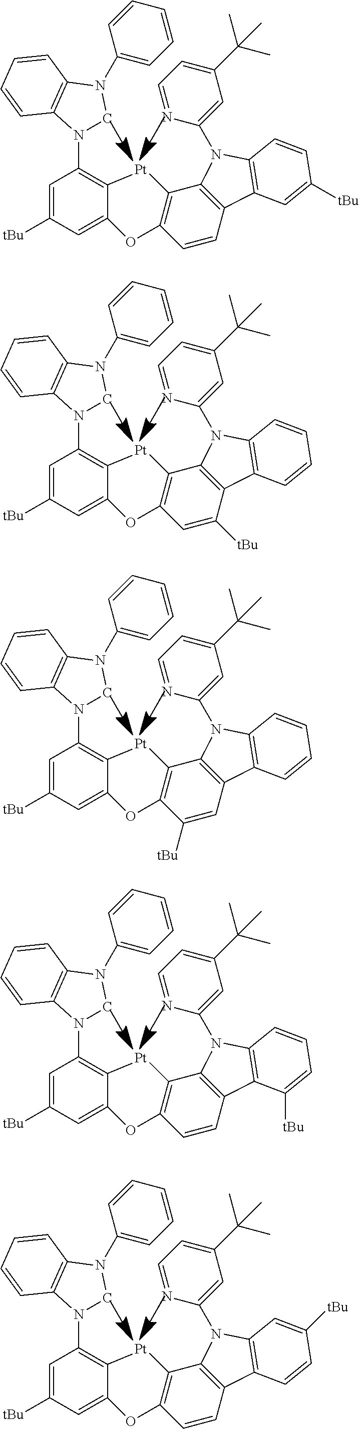

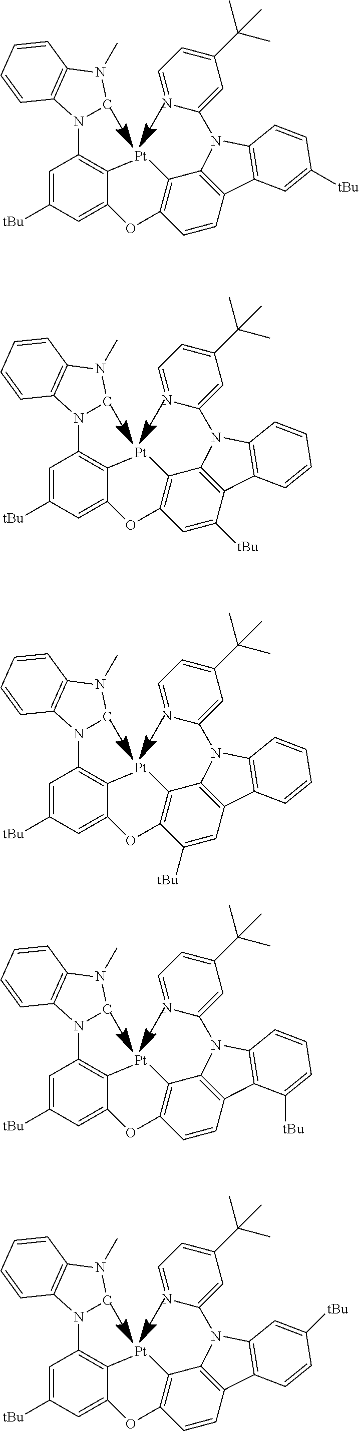

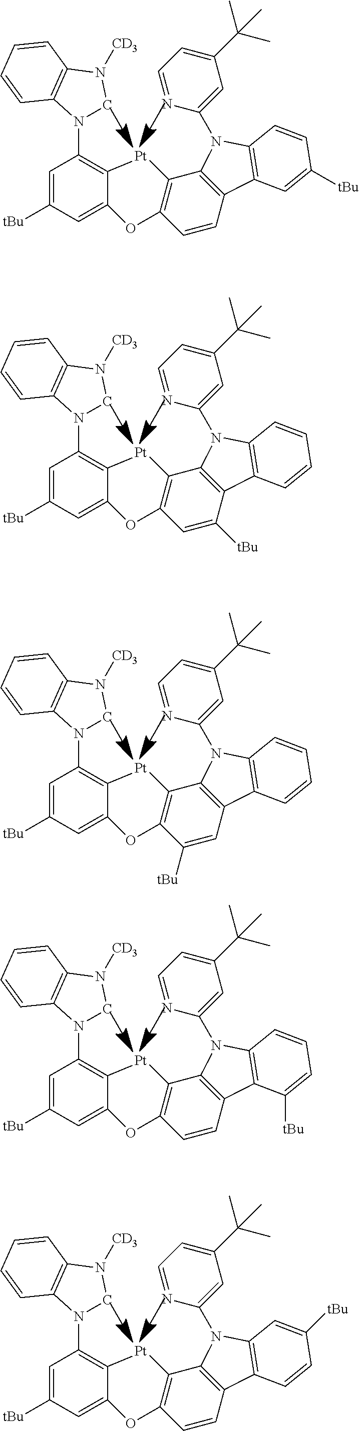

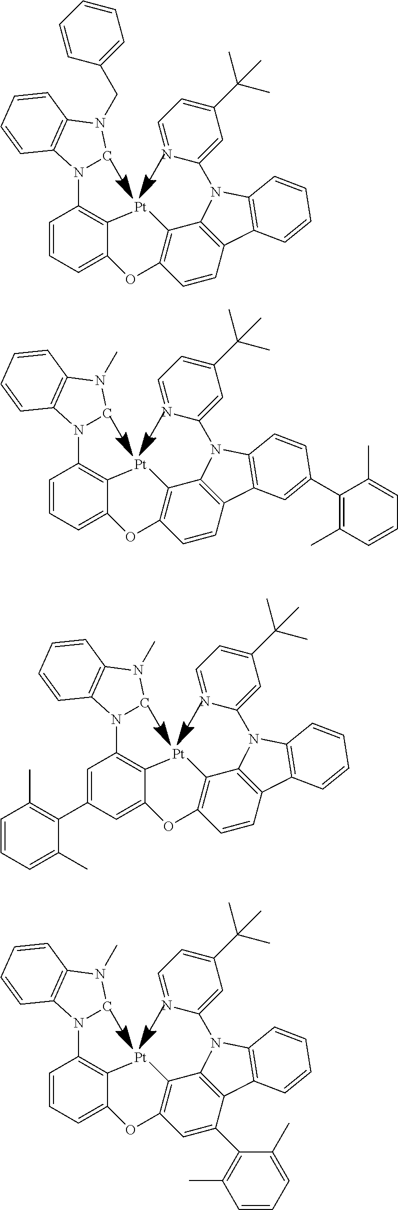

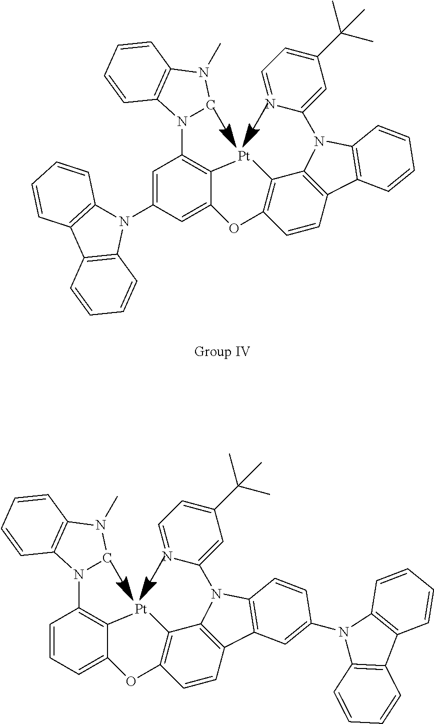

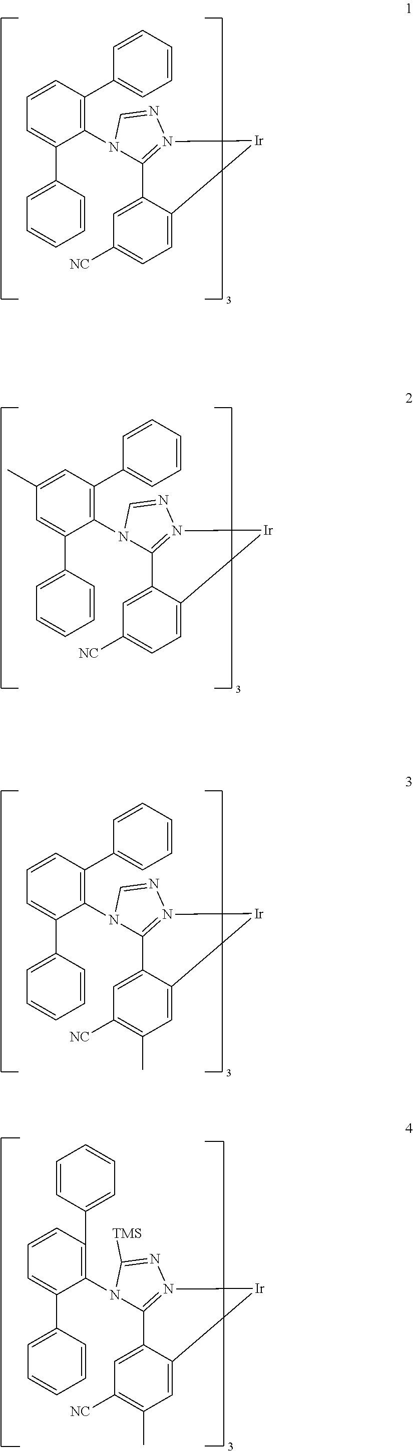

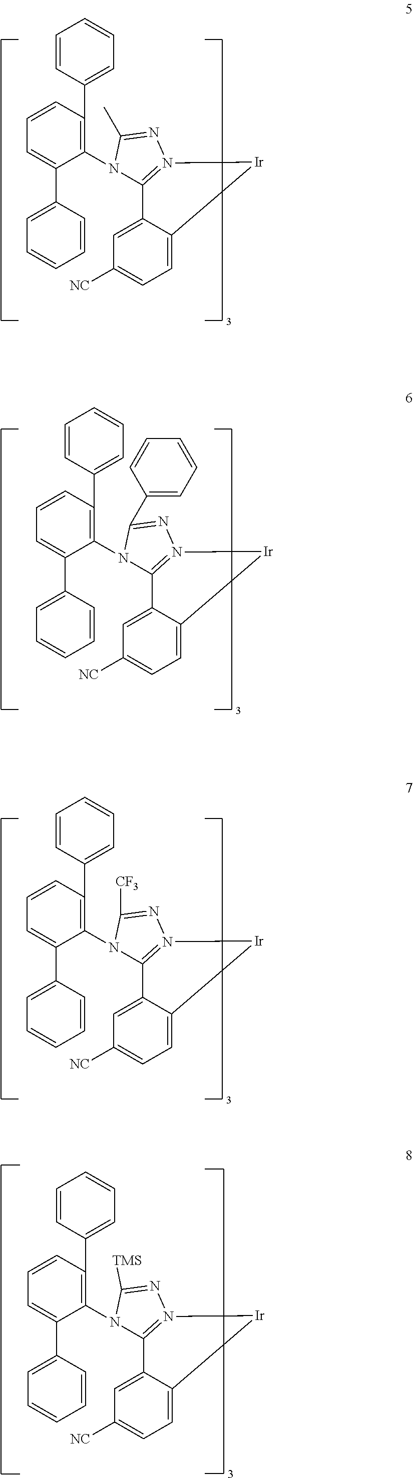

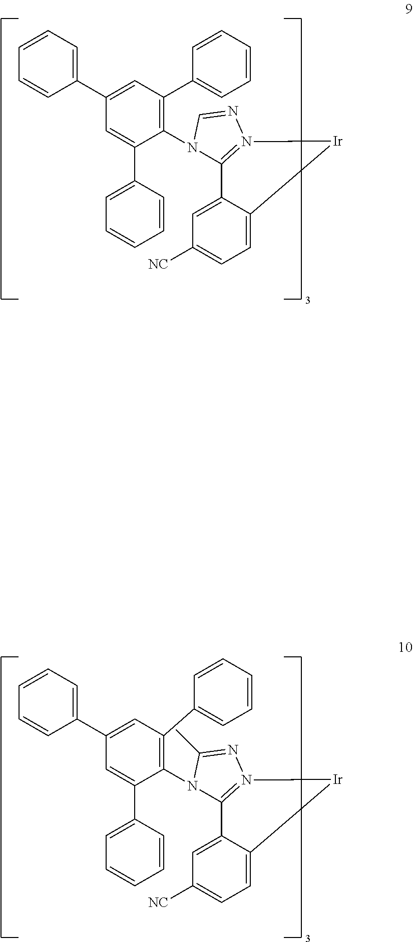

Compounds of Group V include a compound represented by Formula A below:

(L₁₀₁)ₙ₁₀₁-M₁₀₁-(L₁₀₂)m₁₀₁ Formula A

L₁₀₁, n101, M₁₀₁, L₁₀₂, and M₁₀₁ in Formula A are the same as described in connection with Tables 2 to 4:

TABLE 2

| Name of compound | L₁₀₁ | n101 | M₁₀₁ | L₁₀₂ | m101 |
|---|---|---|---|---|---|
| BD001 | LM1 | 3 | Ir | — | 0 |
| BD002 | LM2 | 3 | Ir | — | 0 |
| BD003 | LM3 | 3 | Ir | — | 0 |
| BD004 | LM4 | 3 | Ir | — | 0 |
| BD005 | LM5 | 3 | Ir | — | 0 |
| BD006 | LM6 | 3 | Ir | — | 0 |
| BD007 | LM7 | 3 | Ir | — | 0 |
| BD008 | LM8 | 3 | Ir | — | 0 |
| BD009 | LM9 | 3 | Ir | — | 0 |
| BD010 | LM10 | 3 | Ir | — | 0 |
| BD011 | LM11 | 3 | Ir | — | 0 |
| BD012 | LM12 | 3 | Ir | — | 0 |
| BD013 | LM13 | 3 | Ir | — | 0 |
| BD014 | LM14 | 3 | Ir | — | 0 |
| BD015 | LM15 | 3 | Ir | — | 0 |
| BD016 | LM16 | 3 | Ir | — | 0 |
| BD017 | LM17 | 3 | Ir | — | 0 |
| BD018 | LM18 | 3 | Ir | — | 0 |
| BD019 | LM19 | 3 | Ir | — | 0 |
| BD020 | LM20 | 3 | Ir | — | 0 |
| BD021 | LM21 | 3 | Ir | — | 0 |
| BD022 | LM22 | 3 | Ir | — | 0 |
| BD023 | LM23 | 3 | Ir | — | 0 |
| BD024 | LM24 | 3 | Ir | — | 0 |
| BD025 | LM25 | 3 | Ir | — | 0 |
| BD026 | LM26 | 3 | Ir | — | 0 |
| BD027 | LM27 | 3 | Ir | — | 0 |
| BD028 | LM28 | 3 | Ir | — | 0 |
| BD029 | LM29 | 3 | Ir | — | 0 |
| BD030 | LM30 | 3 | Ir | — | 0 |
| BD031 | LM31 | 3 | Ir | — | 0 |
| BD032 | LM32 | 3 | Ir | — | 0 |
| BD033 | LM33 | 3 | Ir | — | 0 |
| BD034 | LM34 | 3 | Ir | — | 0 |
| BD035 | LM35 | 3 | Ir | — | 0 |
| BD036 | LM36 | 3 | Ir | — | 0 |
| BD037 | LM37 | 3 | Ir | — | 0 |
| BD038 | LM38 | 3 | Ir | — | 0 |
| BD039 | LM39 | 3 | Ir | — | 0 |
| BD040 | LM40 | 3 | Ir | — | 0 |
| BD041 | LM41 | 3 | Ir | — | 0 |
| BD042 | LM42 | 3 | Ir | — | 0 |
| BD043 | LM43 | 3 | Ir | — | 0 |
| BD044 | LM44 | 3 | Ir | — | 0 |
| BD045 | LM45 | 3 | Ir | — | 0 |
| BD046 | LM46 | 3 | Ir | — | 0 |
| BD047 | LM47 | 3 | Ir | — | 0 |
| BD048 | LM48 | 3 | Ir | — | 0 |
| BD049 | LM49 | 3 | Ir | — | 0 |
| BD050 | LM50 | 3 | Ir | — | 0 |
| BD051 | LM51 | 3 | Ir | — | 0 |
| BD052 | LM52 | 3 | Ir | — | 0 |
| BD053 | LM53 | 3 | Ir | — | 0 |
| BD054 | LM54 | 3 | Ir | — | 0 |
| BD055 | LM55 | 3 | Ir | — | 0 |
| BD056 | LM56 | 3 | Ir | — | 0 |
| BD057 | LM57 | 3 | Ir | — | 0 |
| BD058 | LM58 | 3 | Ir | — | 0 |
| BD059 | LM59 | 3 | Ir | — | 0 |
| BD060 | LM60 | 3 | Ir | — | 0 |
| BD061 | LM61 | 3 | Ir | — | 0 |
| BD062 | LM62 | 3 | Ir | — | 0 |
| BD063 | LM63 | 3 | Ir | — | 0 |
| BD064 | LM64 | 3 | Ir | — | 0 |
| BD065 | LM65 | 3 | Ir | — | 0 |
| BD066 | LM66 | 3 | Ir | — | 0 |
| BD067 | LM67 | 3 | Ir | — | 0 |
| BD068 | LM68 | 3 | Ir | — | 0 |
| BD069 | LM69 | 3 | Ir | — | 0 |
| BD070 | LM70 | 3 | Ir | — | 0 |
| BD071 | LM71 | 3 | Ir | — | 0 |
| BD072 | LM72 | 3 | Ir | — | 0 |
| BD073 | LM73 | 3 | Ir | — | 0 |
| BD074 | LM74 | 3 | Ir | — | 0 |
| BD075 | LM75 | 3 | Ir | — | 0 |
| BD076 | LM76 | 3 | Ir | — | 0 |
| BD077 | LM77 | 3 | Ir | — | 0 |
| BD078 | LM78 | 3 | Ir | — | 0 |
| BD079 | LM79 | 3 | Ir | — | 0 |
| BD080 | LM80 | 3 | Ir | — | 0 |
| BD081 | LM81 | 3 | Ir | — | 0 |
| BD082 | LM82 | 3 | Ir | — | 0 |
| BD083 | LM83 | 3 | Ir | — | 0 |
| BD084 | LM84 | 3 | Ir | — | 0 |
| BD085 | LM85 | 3 | Ir | — | 0 |
| BD086 | LM86 | 3 | Ir | — | 0 |
| BD087 | LM87 | 3 | Ir | — | 0 |
| BD088 | LM88 | 3 | Ir | — | 0 |
| BD089 | LM89 | 3 | Ir | — | 0 |
| BD090 | LM90 | 3 | Ir | — | 0 |
| BD091 | LM91 | 3 | Ir | — | 0 |
| BD092 | LM92 | 3 | Ir | — | 0 |
| BD093 | LM93 | 3 | Ir | — | 0 |
| BD094 | LM94 | 3 | Ir | — | 0 |
| BD095 | LM95 | 3 | Ir | — | 0 |
| BD096 | LM96 | 3 | Ir | — | 0 |
| BD097 | LM97 | 3 | Ir | — | 0 |
| BD098 | LM98 | 3 | Ir | — | 0 |
| BD099 | LM99 | 3 | Ir | — | 0 |
| BD100 | LM100 | 3 | Ir | — | 0 |

TABLE 3

| Name of compound | L₁₀₁ | n101 | M₁₀₁ | L₁₀₂ | m101 |
|---|---|---|---|---|---|
| BD101 | LM101 | 3 | Ir | — | 0 |
| BD102 | LM102 | 3 | Ir | — | 0 |
| BD103 | LM103 | 3 | Ir | — | 0 |
| BD104 | LM104 | 3 | Ir | — | 0 |
| BD105 | LM105 | 3 | Ir | — | 0 |
| BD106 | LM106 | 3 | Ir | — | 0 |
| BD107 | LM107 | 3 | Ir | — | 0 |
| BD108 | LM108 | 3 | Ir | — | 0 |
| BD109 | LM109 | 3 | Ir | — | 0 |
| BD110 | LM110 | 3 | Ir | — | 0 |
| BD111 | LM111 | 3 | Ir | — | 0 |
| BD112 | LM112 | 3 | Ir | — | 0 |
| BD113 | LM113 | 3 | Ir | — | 0 |
| BD114 | LM114 | 3 | Ir | — | 0 |
| BD115 | LM115 | 3 | Ir | — | 0 |
| BD116 | LM116 | 3 | Ir | — | 0 |
| BD117 | LM117 | 3 | Ir | — | 0 |
| BD118 | LM118 | 3 | Ir | — | 0 |
| BD119 | LM119 | 3 | Ir | — | 0 |
| BD120 | LM120 | 3 | Ir | — | 0 |
| BD121 | LM121 | 3 | Ir | — | 0 |
| BD122 | LM122 | 3 | Ir | — | 0 |
| BD123 | LM123 | 3 | Ir | — | 0 |
| BD124 | LM124 | 3 | Ir | — | 0 |
| BD125 | LM125 | 3 | Ir | — | 0 |
| BD126 | LM126 | 3 | Ir | — | 0 |
| BD127 | LM127 | 3 | Ir | — | 0 |
| BD128 | LM128 | 3 | Ir | — | 0 |
| BD129 | LM129 | 3 | Ir | — | 0 |
| BD130 | LM130 | 3 | lr | — | 0 |
| BD131 | LM131 | 3 | Ir | — | 0 |
| BD132 | LM132 | 3 | lr | — | 0 |
| BD133 | LM133 | 3 | Ir | — | 0 |
| BD134 | LM134 | 3 | Ir | — | 0 |
| BD135 | LM135 | 3 | Ir | — | 0 |
| BD136 | LM136 | 3 | Ir | — | 0 |
| BD137 | LM137 | 3 | Ir | — | 0 |
| BD138 | LM138 | 3 | Ir | — | 0 |
| BD139 | LM139 | 3 | Ir | — | 0 |
| BD140 | LM140 | 3 | Ir | — | 0 |
| BD141 | LM141 | 3 | Ir | — | 0 |
| BD142 | LM142 | 3 | Ir | — | 0 |
| BD143 | LM143 | 3 | Ir | — | 0 |
| BD144 | LM144 | 3 | Ir | — | 0 |
| BD145 | LM145 | 3 | Ir | — | 0 |
| BD146 | LM146 | 3 | Ir | — | 0 |
| BD147 | LM147 | 3 | Ir | — | 0 |
| BD148 | LM148 | 3 | Ir | — | 0 |
| BD149 | LM149 | 3 | Ir | — | 0 |
| BD150 | LM150 | 3 | Ir | — | 0 |
| BD151 | LM151 | 3 | Ir | — | 0 |
| BD152 | LM152 | 3 | Ir | — | 0 |
| BD153 | LM153 | 3 | Ir | — | 0 |
| BD154 | LM154 | 3 | Ir | — | 0 |
| BD155 | LM155 | 3 | Ir | — | 0 |
| BD156 | LM156 | 3 | Ir | — | 0 |
| BD157 | LM157 | 3 | Ir | — | 0 |
| BD158 | LM158 | 3 | Ir | — | 0 |
| BD159 | LM159 | 3 | Ir | — | 0 |
| BD160 | LM160 | 3 | Ir | — | 0 |
| BD161 | LM161 | 3 | Ir | — | 0 |
| BD162 | LM162 | 3 | Ir | — | 0 |
| BD163 | LM163 | 3 | Ir | — | 0 |
| BD164 | LM164 | 3 | Ir | — | 0 |
| BD165 | LM165 | 3 | Ir | — | 0 |
| BD166 | LM166 | 3 | Ir | — | 0 |
| BD167 | LM167 | 3 | Ir | — | 0 |
| BD168 | LM168 | 3 | Ir | — | 0 |
| BD169 | LM169 | 3 | Ir | — | 0 |
| BD170 | LM170 | 3 | Ir | — | 0 |
| BD171 | LM171 | 3 | Ir | — | 0 |
| BD172 | LM172 | 3 | Ir | — | 0 |
| BD173 | LM173 | 3 | Ir | — | 0 |
| BD174 | LM174 | 3 | Ir | — | 0 |
| BD175 | LM175 | 3 | Ir | — | 0 |
| BD176 | LM176 | 3 | Ir | — | 0 |
| BD177 | LM177 | 3 | Ir | — | 0 |
| BD178 | LM178 | 3 | Ir | — | 0 |
| BD179 | LM179 | 3 | Ir | — | 0 |
| BD180 | LM180 | 3 | Ir | — | 0 |
| BD181 | LM181 | 3 | Ir | — | 0 |
| BD182 | LM182 | 3 | Ir | — | 0 |
| BD183 | LM183 | 3 | Ir | — | 0 |
| BD184 | LM184 | 3 | Ir | — | 0 |
| BD185 | LM185 | 3 | Ir | — | 0 |
| BD186 | LM186 | 3 | Ir | — | 0 |
| BD187 | LM187 | 3 | Ir | — | 0 |
| BD188 | LM188 | 3 | Ir | — | 0 |
| BD189 | LM189 | 3 | Ir | — | 0 |
| BD190 | LM190 | 3 | Ir | — | 0 |
| BD191 | LM191 | 3 | Ir | — | 0 |
| BD192 | LM192 | 3 | Ir | — | 0 |
| BD193 | LM193 | 3 | Ir | — | 0 |
| BD194 | LM194 | 3 | Ir | — | 0 |
| BD195 | LM195 | 3 | Ir | — | 0 |
| BD196 | LM196 | 3 | Ir | — | 0 |
| BD197 | LM197 | 3 | Ir | — | 0 |
| BD198 | LM198 | 3 | Ir | — | 0 |
| BD199 | LM199 | 3 | Ir | — | 0 |
| BD200 | LM200 | 3 | Ir | — | 0 |

TABLE 4

| Name of compound | L₁₀₁ | n101 | M₁₀₁ | L₁₀₂ | m101 |
|---|---|---|---|---|---|
| BD201 | LM201 | 3 | Ir | — | 0 |
| BD202 | LM202 | 3 | Ir | — | 0 |
| BD203 | LM203 | 3 | Ir | — | 0 |
| BD204 | LM204 | 3 | Ir | — | 0 |
| BD205 | LM205 | 3 | Ir | — | 0 |
| BD206 | LM206 | 3 | Ir | — | 0 |
| BD207 | LM207 | 3 | Ir | — | 0 |
| BD208 | LM208 | 3 | Ir | — | 0 |
| BD209 | LM209 | 3 | Ir | — | 0 |
| BD210 | LM210 | 3 | Ir | — | 0 |
| BD211 | LM211 | 3 | Ir | — | 0 |
| BD212 | LM212 | 3 | Ir | — | 0 |
| BD213 | LM213 | 3 | lr | — | 0 |
| BD214 | LM214 | 3 | Ir | — | 0 |
| BD215 | LM215 | 3 | lr | — | 0 |
| BD216 | LM216 | 3 | Ir | — | 0 |
| BD217 | LM217 | 3 | Ir | — | 0 |
| BD218 | LM218 | 3 | Ir | — | 0 |
| BD219 | LM219 | 3 | Ir | — | 0 |
| BD220 | LM220 | 3 | Ir | — | 0 |
| BD221 | LM221 | 3 | Ir | — | 0 |
| BD222 | LM222 | 3 | Ir | — | 0 |
| BD223 | LM223 | 3 | Ir | — | 0 |
| BD224 | LM224 | 3 | Ir | — | 0 |
| BD225 | LM225 | 3 | Ir | — | 0 |
| BD226 | LM226 | 3 | Ir | — | 0 |
| BD227 | LM227 | 3 | Ir | — | 0 |
| BD228 | LM228 | 3 | Ir | — | 0 |
| BD229 | LM229 | 3 | Ir | — | 0 |
| BD230 | LM230 | 3 | Ir | — | 0 |
| BD231 | LM231 | 3 | Ir | — | 0 |
| BD232 | LM232 | 3 | Ir | — | 0 |
| BD233 | LM233 | 3 | Ir | — | 0 |
| BD234 | LM234 | 3 | Ir | — | 0 |
| BD235 | LM235 | 3 | Ir | — | 0 |
| BD236 | LM236 | 3 | Ir | — | 0 |
| BD237 | LM237 | 3 | Ir | — | 0 |
| BD238 | LM238 | 3 | Ir | — | 0 |
| BD239 | LM239 | 3 | Ir | — | 0 |
| BD240 | LM240 | 3 | Ir | — | 0 |
| BD241 | LM241 | 3 | Ir | — | 0 |
| BD242 | LM242 | 3 | Ir | — | 0 |
| BD243 | LM243 | 3 | Ir | — | 0 |
| BD244 | LFM1 | 3 | Ir | — | 0 |
| BD245 | LFM2 | 3 | Ir | — | 0 |
| BD246 | LFM3 | 3 | Ir | — | 0 |
| BD247 | LFM4 | 3 | Ir | — | 0 |
| BD248 | LFM5 | 3 | Ir | — | 0 |
| BD249 | LFM6 | 3 | Ir | — | 0 |
| BD250 | LFM7 | 3 | Ir | — | 0 |
| BD251 | LFP1 | 3 | Ir | — | 0 |
| BD252 | LFP2 | 3 | Ir | — | 0 |
| BD253 | LFP3 | 3 | Ir | — | 0 |
| BD254 | LFP4 | 3 | Ir | — | 0 |
| BD255 | LFP5 | 3 | Ir | — | 0 |
| BD256 | LFP6 | 3 | Ir | — | 0 |
| BD257 | LFP7 | 3 | Ir | — | 0 |
| BD258 | LM47 | 2 | Ir | AN1 | 1 |
| BD259 | LM47 | 2 | Ir | AN2 | 1 |
| BD260 | LM47 | 2 | Ir | AN3 | 1 |
| BD261 | LM47 | 2 | Ir | AN4 | 1 |
| BD262 | LM47 | 2 | Ir | AN5 | 1 |
| BD263 | LM11 | 2 | Pt | — | 0 |
| BD264 | LM13 | 2 | Pt | — | 0 |
| BD265 | LM15 | 2 | Pt | — | 0 |
| BD266 | LM45 | 2 | Pt | — | 0 |
| BD267 | LM47 | 2 | Pt | — | 0 |
| BD268 | LM49 | 2 | Pt | — | 0 |
| BD269 | LM98 | 2 | Pt | — | 0 |
| BD270 | LM100 | 2 | Pt | — | 0 |
| BD271 | LM102 | 2 | Pt | — | 0 |
| BD272 | LM132 | 2 | Pt | — | 0 |
| BD273 | LM134 | 2 | Pt | — | 0 |
| BD274 | LM136 | 2 | Pt | — | 0 |
| BD275 | LM151 | 2 | Pt | — | 0 |
| BD276 | LM153 | 2 | Pt | — | 0 |
| BD277 | LM158 | 2 | Pt | — | 0 |
| BD278 | LM180 | 2 | Pt | — | 0 |
| BD279 | LM182 | 2 | Pt | — | 0 |
| BD280 | LM187 | 2 | Pt | — | 0 |
| BD281 | LM201 | 2 | Pt | — | 0 |
| BD282 | LM206 | 2 | Pt | — | 0 |
| BD283 | LM211 | 2 | Pt | — | 0 |
| BD284 | LM233 | 2 | Pt | — | 0 |
| BD285 | LM235 | 2 | Pt | — | 0 |
| BD286 | LM240 | 2 | Pt | — | 0 |
| BD287 | LFM5 | 2 | Pt | — | 0 |
| BD288 | LFM6 | 2 | Pt | — | 0 |
| BD289 | LFM7 | 2 | Pt | — | 0 |
| BD290 | LFP5 | 2 | Pt | — | 0 |
| BD291 | LFP6 | 2 | Pt | — | 0 |
| BD292 | LFP7 | 2 | Pt | — | 0 |
| BD293 | LM47 | 1 | Pt | AN1 | 1 |
| BD294 | LM47 | 1 | Pt | AN2 | 1 |
| BD295 | LM47 | 1 | Pt | AN3 | 1 |
| BD296 | LM47 | 1 | Pt | AN4 | 1 |
| BD297 | LM47 | 1 | Pt | AN5 | 1 |

Regarding Tables 2 to 4, LM1 to LM243, LFM1 to LFM7 and LFP1 to LFP8 may be understood with reference to Formulae 1-1 to 1-3 and Tables 5 to 7:

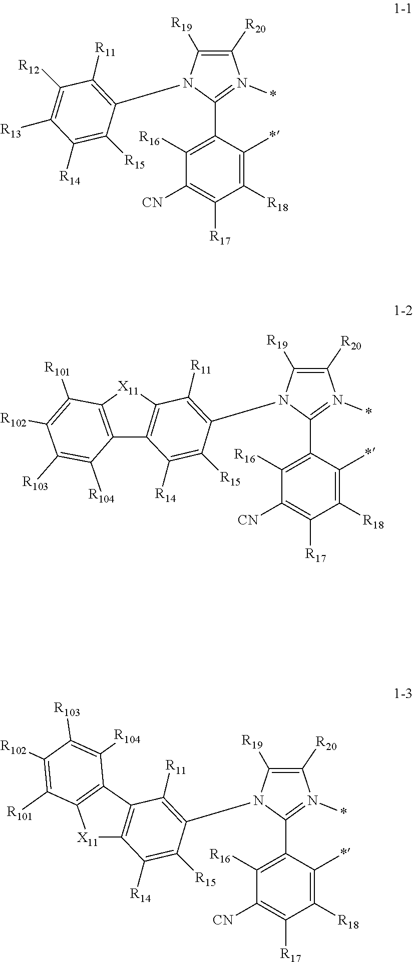

TABLE 5
Formula 1-1

| Name of ligand | R₁₁ | R₁₂ | R₁₃ | R₁₄ | R₁₅ | R₁₆ | R₁₇ | R₁₈ | R₁₉ | R₂₀ |
|---|---|---|---|---|---|---|---|---|---|---|
| LM1 | X1 | H | X3 | H | X1 | H | H | H | H | D |
| LM2 | X1 | H | X3 | H | X1 | H | H | H | D | H |
| LM3 | X1 | H | X3 | H | X1 | H | H | H | D | D |
| LM4 | Y1 | H | X3 | H | Y1 | H | H | H | D | D |
| LM5 | Y2 | H | X3 | H | Y2 | H | H | H | D | D |
| LM6 | Y3 | H | X3 | H | Y3 | H | H | H | D | D |
| LM7 | Y3 | D | X3 | D | Y3 | H | H | H | D | D |
| LM8 | Y3 | D | X3 | D | Y3 | D | H | H | D | D |
| LM9 | Y3 | D | X3 | D | Y3 | D | D | H | D | D |
| LM10 | Y3 | D | X3 | D | Y3 | D | D | D | D | D |
| LM11 | Y3 | D | Y11 | D | Y3 | D | D | D | D | D |
| LM12 | Y3 | D | Y11 | D | Y3 | H | X1 | H | D | D |
| LM13 | Y3 | D | Y11 | D | Y3 | D | Y3 | D | D | D |
| LM14 | Y3 | D | Y11 | D | Y3 | H | X4 | H | D | D |
| LM15 | Y3 | D | Y11 | D | Y3 | D | Y12 | D | D | D |
| LM16 | X2 | H | X3 | H | X2 | H | H | H | H | D |
| LM17 | X2 | H | X3 | H | X2 | H | H | H | D | H |
| LM18 | X2 | H | X3 | H | X2 | H | H | H | D | D |
| LM19 | Y4 | H | X3 | H | Y4 | H | H | H | D | D |
| LM20 | Y5 | H | X3 | H | Y5 | H | H | H | D | D |
| LM21 | Y6 | H | X3 | H | Y6 | H | H | H | D | D |
| LM22 | Y7 | H | X3 | H | Y7 | H | H | H | D | D |
| LM23 | Y8 | H | X3 | H | Y8 | H | H | H | D | D |
| LM24 | Y9 | H | X3 | H | Y9 | H | H | H | D | D |
| LM25 | Y10 | H | X3 | H | Y10 | H | H | H | D | D |
| LM26 | Y10 | D | X3 | D | Y10 | H | H | H | D | D |
| LM27 | Y10 | D | X3 | D | Y10 | D | H | H | D | D |
| LM28 | Y10 | D | X3 | D | Y10 | D | D | H | D | D |
| LM29 | Y10 | D | X3 | D | Y10 | D | D | D | D | D |
| LM30 | Y10 | D | Y11 | D | Y10 | D | D | D | D | D |
| LM31 | Y10 | D | Y11 | D | Y10 | H | X1 | H | D | D |
| LM32 | Y10 | D | Y11 | D | Y10 | D | Y3 | D | D | D |
| LM33 | Y10 | D | Y11 | D | Y10 | H | X4 | H | D | D |
| LM34 | Y10 | D | Y11 | D | Y10 | D | Y12 | D | D | D |
| LM35 | X1 | H | X4 | H | X1 | H | H | H | H | D |
| LM36 | X1 | H | X4 | H | X1 | H | H | H | D | H |
| LM37 | X1 | H | X4 | H | X1 | H | H | H | D | D |
| LM38 | Y1 | H | X4 | H | Y1 | H | H | H | D | D |
| LM39 | Y2 | H | X4 | H | Y2 | H | H | H | D | D |
| LM40 | Y3 | H | X4 | H | Y3 | H | H | H | D | D |
| LM41 | Y3 | D | X4 | D | Y3 | H | H | H | D | D |
| LM42 | Y3 | D | X4 | D | Y3 | D | H | H | D | D |
| LM43 | Y3 | D | X4 | D | Y3 | D | D | H | D | D |
| LM44 | Y3 | D | X4 | D | Y3 | D | D | D | D | D |
| LM45 | Y3 | D | Y12 | D | Y3 | D | D | D | D | D |
| LM46 | Y3 | D | Y12 | D | Y3 | H | X1 | H | D | D |
| LM47 | Y3 | D | Y12 | D | Y3 | D | Y3 | D | D | D |
| LM48 | Y3 | D | Y12 | D | Y3 | H | X4 | H | D | D |
| LM49 | Y3 | D | Y12 | D | Y3 | D | Y12 | D | D | D |
| LM50 | X2 | H | X4 | H | X2 | H | H | H | H | D |
| LM51 | X2 | H | X4 | H | X2 | H | H | H | D | H |
| LM52 | X2 | H | X4 | H | X2 | H | H | H | D | D |
| LM53 | Y4 | H | X4 | H | Y4 | H | H | H | D | D |
| LM54 | Y5 | H | X4 | H | Y5 | H | H | H | D | D |
| LM55 | Y6 | H | X4 | H | Y6 | H | H | H | D | D |
| LM56 | Y7 | H | X4 | H | Y7 | H | H | H | D | D |
| LM57 | Y8 | H | X4 | H | Y8 | H | H | H | D | D |
| LM58 | Y9 | H | X4 | H | Y9 | H | H | H | D | D |
| LM59 | Y10 | H | X4 | H | Y10 | H | H | H | D | D |
| LM60 | Y10 | D | X4 | D | Y10 | H | H | H | D | D |
| LM61 | Y10 | D | X4 | D | Y10 | D | H | H | D | D |
| LM62 | Y10 | D | X4 | D | Y10 | D | D | H | D | D |
| LM63 | Y10 | D | X4 | D | Y10 | D | D | D | D | D |
| LM64 | Y10 | D | Y12 | D | Y10 | D | D | D | D | D |
| LM65 | Y10 | D | Y12 | D | Y10 | H | X1 | H | D | D |
| LM66 | Y10 | D | Y12 | D | Y10 | D | Y3 | D | D | D |
| LM67 | Y10 | D | Y12 | D | Y10 | H | X4 | H | D | D |
| LM68 | Y10 | D | Y12 | D | Y10 | D | Y12 | D | D | D |
| LM69 | X1 | H | X5 | H | X1 | H | H | H | H | D |
| LM70 | X1 | H | X5 | H | X1 | H | H | H | D | H |
| LM71 | X1 | H | X5 | H | X1 | H | H | H | D | D |
| LM72 | Y1 | H | X5 | H | Y1 | H | H | H | D | D |
| LM73 | Y2 | H | X5 | H | Y2 | H | H | H | D | D |
| LM74 | Y3 | H | X5 | H | Y3 | H | H | H | D | D |
| LM75 | Y3 | D | X5 | D | Y3 | H | H | H | D | D |
| LM76 | Y3 | D | X5 | D | Y3 | D | H | H | D | D |
| LM77 | Y3 | D | X5 | D | Y3 | D | D | H | D | D |
| LM78 | Y3 | D | X5 | D | Y3 | D | D | D | D | D |
| LM79 | Y3 | D | Y13 | D | Y3 | D | D | D | D | D |
| LM80 | Y3 | D | Y13 | D | Y3 | H | X1 | H | D | D |
| LM81 | Y3 | D | Y13 | D | Y3 | D | Y3 | D | D | D |
| LM82 | Y3 | D | Y13 | D | Y3 | H | X4 | H | D | D |
| LM83 | Y3 | D | Y13 | D | Y3 | D | Y12 | D | D | D |
| LM84 | X2 | H | X5 | H | X2 | H | H | H | H | D |
| LM85 | X2 | H | X5 | H | X2 | H | H | H | D | H |
| LM86 | X2 | H | X5 | H | X2 | H | H | H | D | D |
| LM87 | Y4 | H | X5 | H | Y4 | H | H | H | D | D |
| LM88 | Y5 | H | X5 | H | Y5 | H | H | H | D | D |
| LM89 | Y6 | H | X5 | H | Y6 | H | H | H | D | D |
| LM90 | Y7 | H | X5 | H | Y7 | H | H | H | D | D |
| LM91 | Y8 | H | X5 | H | Y8 | H | H | H | D | D |
| LM92 | Y9 | H | X5 | H | Y9 | H | H | H | D | D |
| LM93 | Y10 | H | X5 | H | Y10 | H | H | H | D | D |
| LM94 | Y10 | D | X5 | D | Y10 | H | H | H | D | D |
| LM95 | Y10 | D | X5 | D | Y10 | D | H | H | D | D |
| LM96 | Y10 | D | X5 | D | Y10 | D | D | H | D | D |
| LM97 | Y10 | D | X5 | D | Y10 | D | D | D | D | D |
| LM98 | Y10 | D | Y13 | D | Y10 | D | D | D | D | D |
| LM99 | Y10 | D | Y13 | D | Y10 | H | X1 | H | D | D |
| LM100 | Y10 | D | Y13 | D | Y10 | D | Y3 | D | D | D |
| LM101 | Y10 | D | Y13 | D | Y10 | H | X4 | H | D | D |
| LM102 | Y10 | D | Y13 | D | Y10 | D | Y12 | D | D | D |
| LM103 | X1 | H | X6 | H | X1 | H | H | H | H | D |
| LM104 | X1 | H | X6 | H | X1 | H | H | H | D | H |
| LM105 | X1 | H | X6 | H | X1 | H | H | H | D | D |
| LM106 | Y1 | H | X6 | H | Y1 | H | H | H | D | D |
| LM107 | Y2 | H | X6 | H | Y2 | H | H | H | D | D |
| LM108 | Y3 | H | X6 | H | Y3 | H | H | H | D | D |
| LM109 | Y3 | D | X6 | D | Y3 | H | H | H | D | D |
| LM110 | Y3 | D | X6 | D | Y3 | D | H | H | D | D |
| LM111 | Y3 | D | X6 | D | Y3 | D | D | H | D | D |
| LM112 | Y3 | D | X6 | D | Y3 | D | D | D | D | D |
| LM113 | Y3 | D | Y14 | D | Y3 | D | D | D | D | D |
| LM114 | Y3 | D | Y14 | D | Y3 | H | X1 | H | D | D |
| LM115 | Y3 | D | Y14 | D | Y3 | D | Y3 | D | D | D |
| LM116 | Y3 | D | Y14 | D | Y3 | H | X4 | H | D | D |
| LM117 | Y3 | D | Y14 | D | Y3 | D | Y12 | D | D | D |
| LM118 | X2 | H | X6 | H | X2 | H | H | H | H | D |
| LM119 | X2 | H | X6 | H | X2 | H | H | H | D | H |
| LM120 | X2 | H | X6 | H | X2 | H | H | H | D | D |
| LM121 | Y4 | H | X6 | H | Y4 | H | H | H | D | D |
| LM122 | Y5 | H | X6 | H | Y5 | H | H | H | D | D |
| LM123 | Y6 | H | X6 | H | Y6 | H | H | H | D | D |
| LM124 | Y7 | H | X6 | H | Y7 | H | H | H | D | D |
| LM125 | Y8 | H | X6 | H | Y8 | H | H | H | D | D |
| LM126 | Y9 | H | X6 | H | Y9 | H | H | H | D | D |
| LM127 | Y10 | H | X6 | H | Y10 | H | H | H | D | D |
| LM128 | Y10 | D | X6 | D | Y10 | H | H | H | D | D |
| LM129 | Y10 | D | X6 | D | Y10 | D | H | H | D | D |
| LM130 | Y10 | D | X6 | D | Y10 | D | D | H | D | D |
| LM131 | Y10 | D | X6 | D | Y10 | D | D | D | D | D |
| LM132 | Y10 | D | Y14 | D | Y10 | D | D | D | D | D |
| LM133 | Y10 | D | Y14 | D | Y10 | H | X1 | H | D | D |
| LM134 | Y10 | D | Y14 | D | Y10 | D | Y3 | D | D | D |
| LM135 | Y10 | D | Y14 | D | Y10 | H | X4 | H | D | D |
| LM136 | Y10 | D | Y14 | D | Y10 | D | Y12 | D | D | D |
| LM137 | X1 | H | X7 | H | X1 | H | H | H | H | D |
| LM138 | X1 | H | X7 | H | X1 | H | H | H | D | H |
| LM139 | X1 | H | X7 | H | X1 | H | H | H | D | D |
| LM140 | Y1 | H | X7 | H | Y1 | H | H | H | D | D |
| LM141 | Y2 | H | X7 | H | Y2 | H | H | H | D | D |
| LM142 | Y3 | H | X7 | H | Y3 | H | H | H | D | D |
| LM143 | Y3 | D | X7 | D | Y3 | H | H | H | D | D |
| LM144 | Y3 | D | X7 | D | Y3 | D | H | H | D | D |
| LM145 | Y3 | D | X7 | D | Y3 | D | D | H | D | D |
| LM146 | Y3 | D | X7 | D | Y3 | D | D | D | D | D |
| LM147 | Y3 | D | X8 | D | Y3 | D | D | D | D | D |
| LM148 | Y3 | D | Y16 | D | Y3 | D | D | D | D | D |
| LM149 | Y3 | D | Y17 | D | Y3 | D | D | D | D | D |
| LM150 | Y3 | D | Y18 | D | Y3 | D | D | D | D | D |
| LM151 | Y3 | D | Y15 | D | Y3 | D | D | D | D | D |
| LM152 | Y3 | D | Y15 | D | Y3 | H | X1 | H | D | D |
| LM153 | Y3 | D | Y15 | D | Y3 | D | Y3 | D | D | D |
| LM154 | Y3 | D | Y16 | D | Y3 | D | Y3 | D | D | D |
| LM155 | Y3 | D | Y17 | D | Y3 | D | Y3 | D | D | D |
| LM156 | Y3 | D | Y18 | D | Y3 | D | Y3 | D | D | D |
| LM157 | Y3 | D | Y15 | D | Y3 | H | X4 | H | D | D |
| LM158 | Y3 | D | Y15 | D | Y3 | D | Y12 | D | D | D |
| LM159 | Y3 | D | Y16 | D | Y3 | D | Y12 | D | D | D |
| LM160 | Y3 | D | Y17 | D | Y3 | D | Y12 | D | D | D |
| LM161 | Y3 | D | Y18 | D | Y3 | D | Y12 | D | D | D |
| LM162 | X2 | H | X7 | H | X2 | H | H | H | H | D |
| LM163 | X2 | H | X7 | H | X2 | H | H | H | D | H |
| LM164 | X2 | H | X7 | H | X2 | H | H | H | D | D |
| LM165 | Y4 | H | X7 | H | Y4 | H | H | H | D | D |
| LM166 | Y5 | H | X7 | H | Y5 | H | H | H | D | D |
| LM167 | Y6 | H | X7 | H | Y6 | H | H | H | D | D |
| LM168 | Y7 | H | X7 | H | Y7 | H | H | H | D | D |
| LM169 | Y8 | H | X7 | H | Y8 | H | H | H | D | D |
| LM170 | Y9 | H | X7 | H | Y9 | H | H | H | D | D |
| LM171 | Y10 | H | X7 | H | Y10 | H | H | H | D | D |
| LM172 | Y10 | D | X7 | D | Y10 | H | H | H | D | D |
| LM173 | Y10 | D | X7 | D | Y10 | D | H | H | D | D |
| LM174 | Y10 | D | X7 | D | Y10 | D | D | H | D | D |
| LM175 | Y10 | D | X7 | D | Y10 | D | D | D | D | D |
| LM176 | Y10 | D | X8 | D | Y10 | D | D | D | D | D |
| LM177 | Y10 | D | Y16 | D | Y10 | D | D | D | D | D |
| LM178 | Y10 | D | Y17 | D | Y10 | D | D | D | D | D |
| LM179 | Y10 | D | Y18 | D | Y10 | D | D | D | D | D |
| LM180 | Y10 | D | Y15 | D | Y10 | D | D | D | D | D |
| LM181 | Y10 | D | Y15 | D | Y10 | H | X1 | H | D | D |
| LM182 | Y10 | D | Y15 | D | Y10 | D | Y3 | D | D | D |
| LM183 | Y10 | D | Y16 | D | Y10 | D | Y3 | D | D | D |
| LM184 | Y10 | D | Y17 | D | Y10 | D | Y3 | D | D | D |
| LM185 | Y10 | D | Y18 | D | Y10 | D | Y3 | D | D | D |
| LM186 | Y10 | D | Y15 | D | Y10 | H | X4 | H | D | D |
| LM187 | Y10 | D | Y15 | D | Y10 | D | Y12 | D | D | D |
| LM188 | Y10 | D | Y16 | D | Y10 | D | Y12 | D | D | D |
| LM189 | Y10 | D | Y17 | D | Y10 | D | Y12 | D | D | D |
| LM190 | Y10 | D | Y18 | D | Y10 | D | Y12 | D | D | D |
| LM191 | X1 | X7 | H | H | X1 | H | H | H | H | D |
| LM192 | X1 | X7 | H | H | X1 | H | H | H | D | H |
| LM193 | X1 | X7 | H | H | X1 | H | H | H | D | D |
| LM194 | Y1 | X7 | H | H | Y1 | H | H | H | D | D |
| LM195 | Y2 | X7 | H | H | Y2 | H | H | H | D | D |
| LM196 | Y3 | X7 | H | H | Y3 | H | H | H | D | D |
| LM197 | Y3 | X7 | D | D | Y3 | H | H | H | D | D |
| LM198 | Y3 | X7 | D | D | Y3 | D | H | H | D | D |
| LM199 | Y3 | X7 | D | D | Y3 | D | D | H | D | D |
| LM200 | Y3 | X7 | D | D | Y3 | D | D | D | D | D |
| LM201 | Y3 | Y15 | D | D | Y3 | D | D | D | D | D |
| LM202 | Y3 | Y16 | D | D | Y3 | D | D | D | D | D |
| LM203 | Y3 | Y17 | D | D | Y3 | D | D | D | D | D |
| LM204 | Y3 | Y18 | D | D | Y3 | D | D | D | D | D |
| LM205 | Y3 | Y15 | D | D | Y3 | H | X1 | H | D | D |
| LM206 | Y3 | Y15 | D | D | Y3 | D | Y3 | D | D | D |
| LM207 | Y3 | Y16 | D | D | Y3 | D | Y3 | D | D | D |
| LM208 | Y3 | Y17 | D | D | Y3 | D | Y3 | D | D | D |
| LM209 | Y3 | Y18 | D | D | Y3 | D | Y3 | D | D | D |
| LM210 | Y3 | Y15 | D | D | Y3 | H | X4 | H | D | D |
| LM211 | Y3 | Y15 | D | D | Y3 | D | Y12 | D | D | D |
| LM212 | Y3 | Y16 | D | D | Y3 | D | Y12 | D | D | D |
| LM213 | Y3 | Y17 | D | D | Y3 | D | Y12 | D | D | D |
| LM214 | Y3 | Y18 | D | D | Y3 | D | Y12 | D | D | D |
| LM215 | X2 | X7 | H | H | X2 | H | H | H | H | D |
| LM216 | X2 | X7 | H | H | X2 | H | H | H | D | H |
| LM217 | X2 | X7 | H | H | X2 | H | H | H | D | D |
| LM218 | Y4 | X7 | H | H | Y4 | H | H | H | D | D |
| LM219 | Y5 | X7 | H | H | Y5 | H | H | H | D | D |
| LM220 | Y6 | X7 | H | H | Y6 | H | H | H | D | D |
| LM221 | Y7 | X7 | H | H | Y7 | H | H | H | D | D |
| LM222 | Y8 | X7 | H | H | Y8 | H | H | H | D | D |
| LM223 | Y9 | X7 | H | H | Y9 | H | H | H | D | D |
| LM224 | Y10 | X7 | H | H | Y10 | H | H | H | D | D |
| LM225 | Y10 | X7 | D | D | Y10 | H | H | H | D | D |
| LM226 | Y10 | X7 | D | D | Y10 | D | H | H | D | D |
| LM227 | Y10 | X7 | D | D | Y10 | D | D | H | D | D |
| LM228 | Y10 | X7 | D | D | Y10 | D | D | D | D | D |
| LM229 | Y10 | X8 | D | D | Y10 | D | D | D | D | D |
| LM230 | Y10 | Y16 | D | D | Y10 | D | D | D | D | D |
| LM231 | Y10 | Y17 | D | D | Y10 | D | D | D | D | D |
| LM232 | Y10 | Y18 | D | D | Y10 | D | D | D | D | D |
| LM233 | Y10 | Y15 | D | D | Y10 | D | D | D | D | D |
| LM234 | Y10 | Y15 | D | D | Y10 | H | X1 | H | D | D |
| LM235 | Y10 | Y15 | D | D | Y10 | D | Y3 | D | D | D |
| LM236 | Y10 | Y16 | D | D | Y10 | D | Y3 | D | D | D |
| LM237 | Y10 | Y17 | D | D | Y10 | D | Y3 | D | D | D |
| LM238 | Y10 | Y18 | D | D | Y10 | D | Y3 | D | D | D |
| LM239 | Y10 | Y15 | D | D | Y10 | H | X4 | H | D | D |
| LM240 | Y10 | Y15 | D | D | Y10 | D | Y12 | D | D | D |
| LM241 | Y10 | Y16 | D | D | Y10 | D | Y12 | D | D | D |
| LM242 | Y10 | Y17 | D | D | Y10 | D | Y12 | D | D | D |
| LM243 | Y10 | Y18 | D | D | Y10 | D | Y12 | D | D | D |

TABLE 6
Formula 1-2

| Name of ligand | R₁₁ | X₁₁ | R₁₀₁ | R₁₀₂ | R₁₀₃ | R₁₀₄ | R₁₄ | R₁₅ | R₁₆ | R₁₇ | R₁₈ | R₁₉ | R₂₀ |
|---|---|---|---|---|---|---|---|---|---|---|---|---|---|
| LFM1 | Y10 | N—Ph | D | D | D | D | D | Y10 | D | D | D | D | D |
| LFM2 | Y10 | S | D | D | D | D | D | Y10 | D | D | D | D | D |
| LFM3 | Y10 | O | D | D | D | D | D | Y10 | D | D | D | D | D |
| LFM4 | Y3 | O | D | D | D | D | D | Y3 | D | D | D | D | D |
| LFM5 | Y10 | O | D | D | D | D | D | Y10 | D | D | D | D | D |
| LFM6 | Y10 | O | D | D | D | D | D | Y10 | D | Y3 | D | D | D |
| LFM7 | Y10 | O | D | D | D | D | D | Y10 | D | Y12 | D | D | D |

TABLE 7
Formula 1-3

| Name of ligand | R₁₁ | X₁₁ | R₁₀₁ | R₁₀₂ | R₁₀₃ | R₁₀₄ | R₁₄ | R₁₅ | R₁₆ | R₁₇ | R₁₈ | R₁₉ | R₂₀ |
|---|---|---|---|---|---|---|---|---|---|---|---|---|---|
| LFP1 | Y10 | N—Ph | D | D | D | D | D | Y10 | D | D | D | D | D |
| LFP2 | Y10 | S | D | D | D | D | D | Y10 | D | D | D | D | D |
| LFP3 | Y10 | O | D | D | D | D | D | Y10 | D | D | D | D | D |
| LFP4 | Y3 | O | D | D | D | D | D | Y3 | D | D | D | D | D |
| LFP5 | Y10 | O | D | D | D | D | D | Y10 | D | D | D | D | D |
| LFP6 | Y10 | O | D | D | D | D | D | Y10 | D | Y3 | D | D | D |

X1 to X10 and Y1 to Y18 in Tables 5-7 are the same as described below, and Ph in the tables refers to a phenyl group:

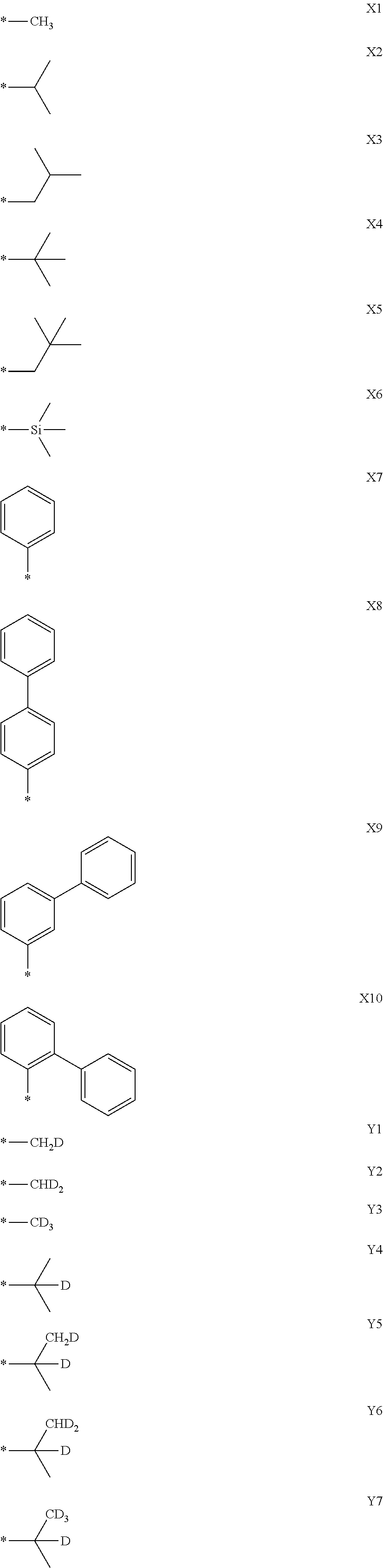

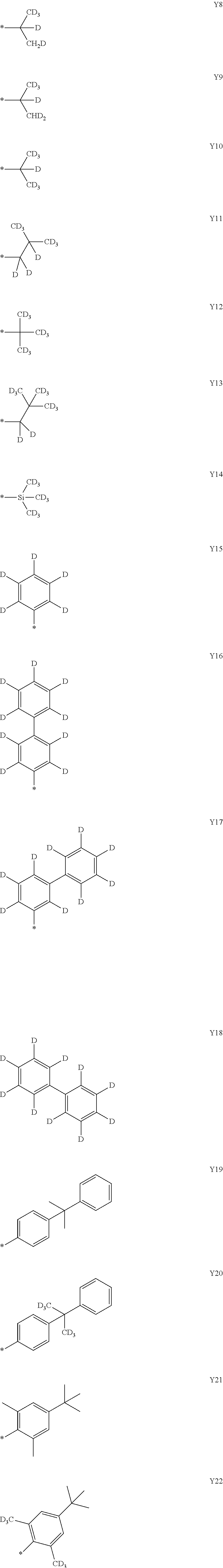

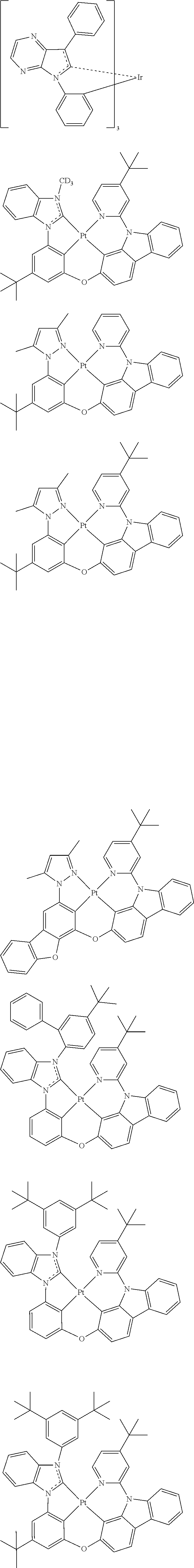

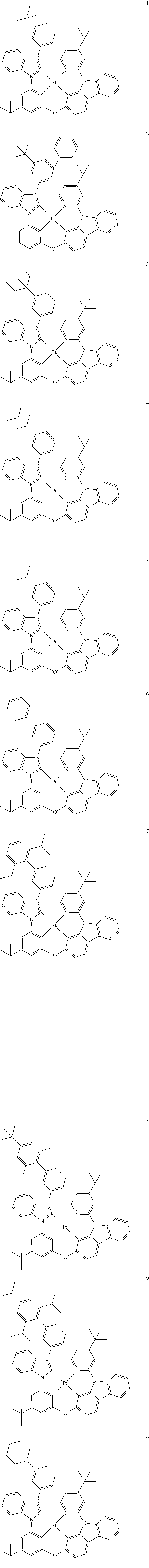

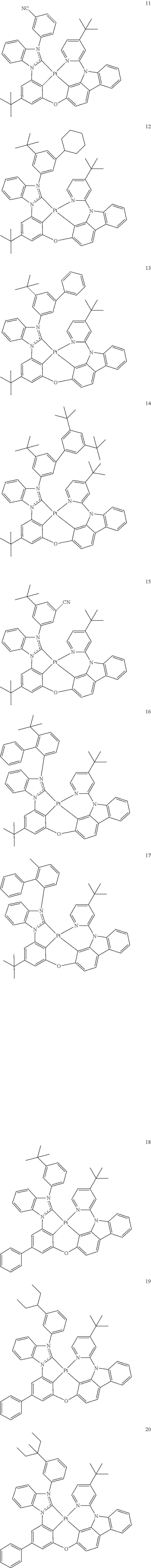

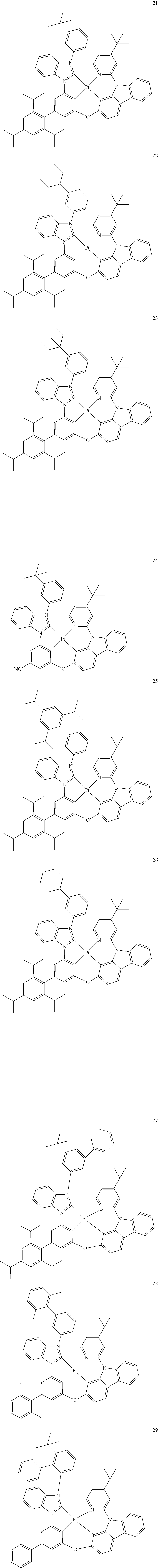

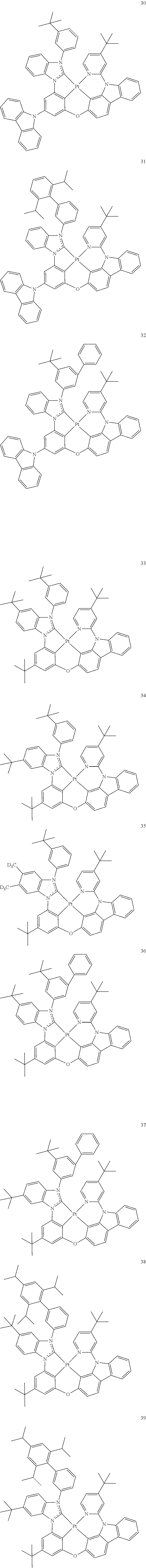

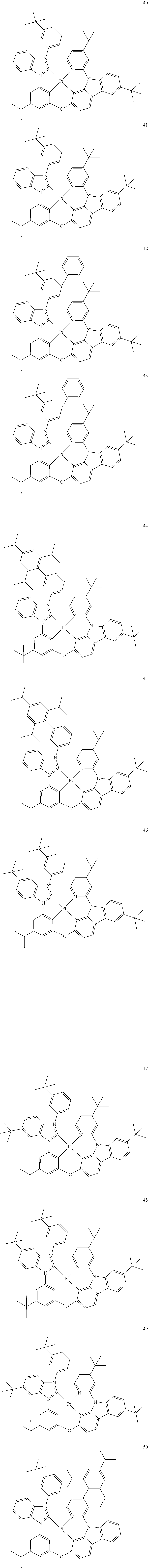

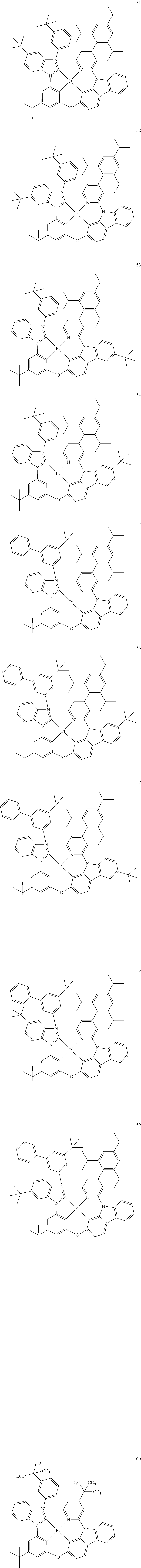

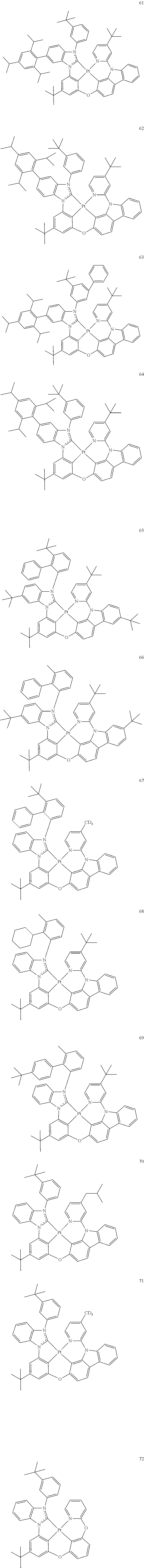

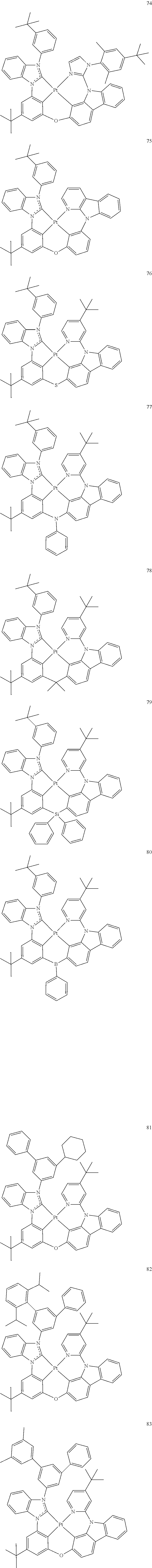

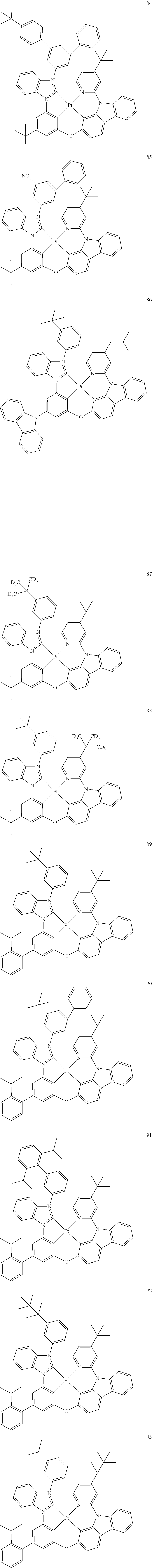

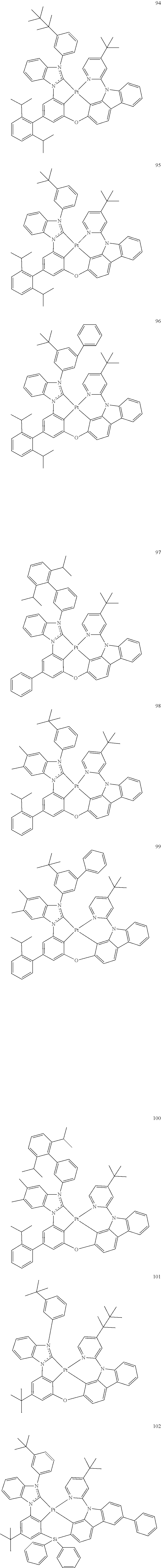

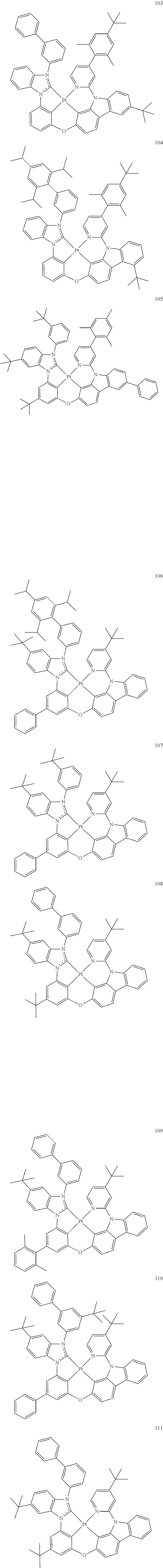

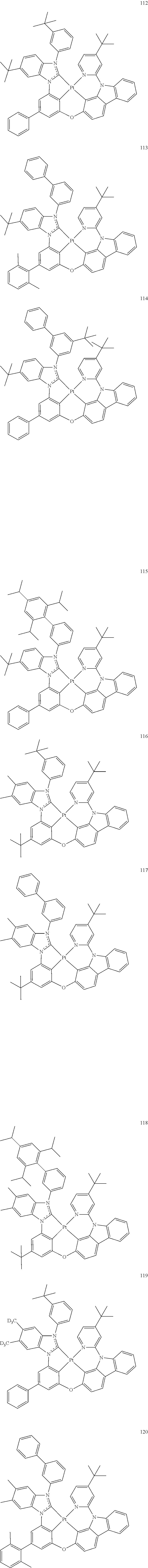

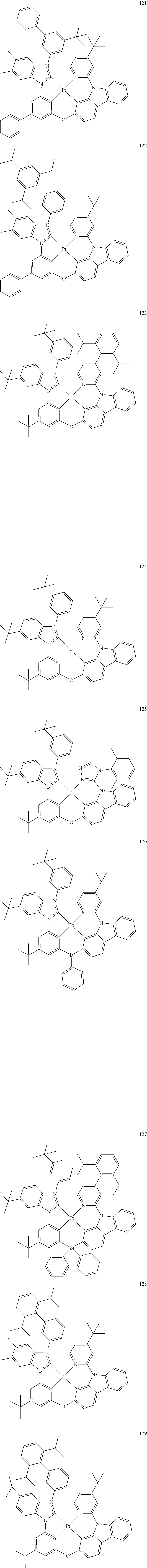

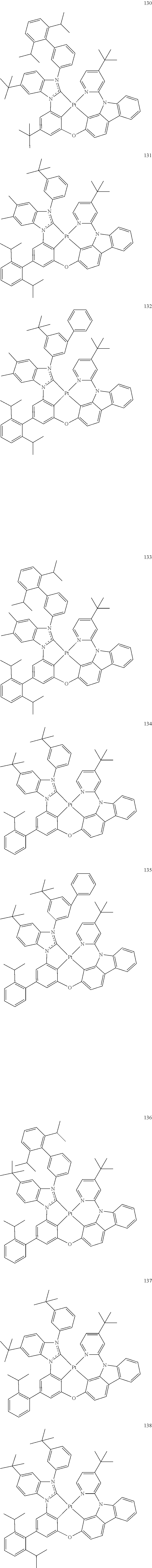

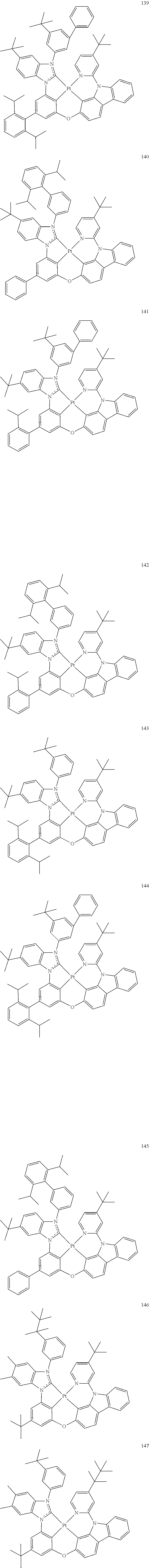

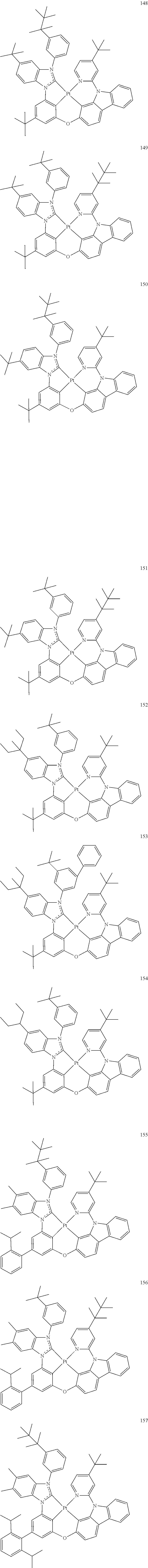

[chemical structure]

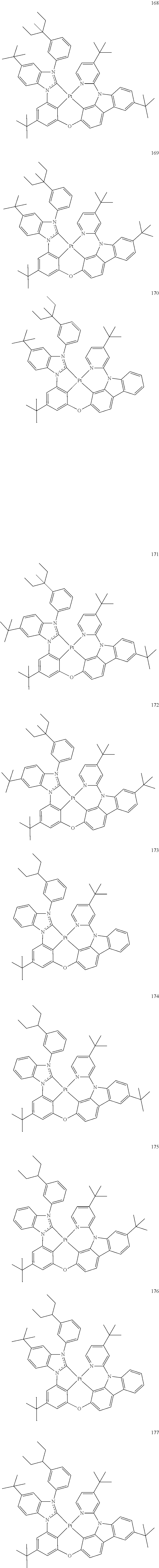

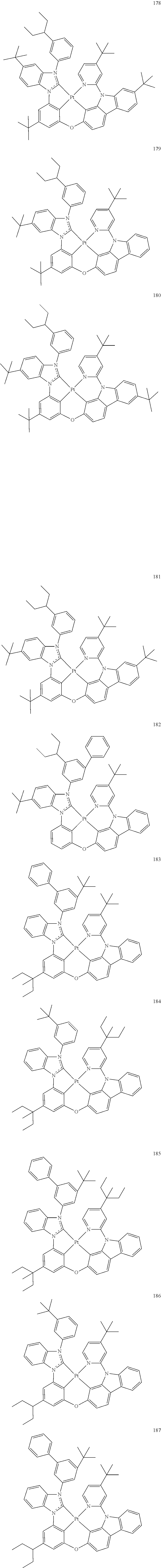

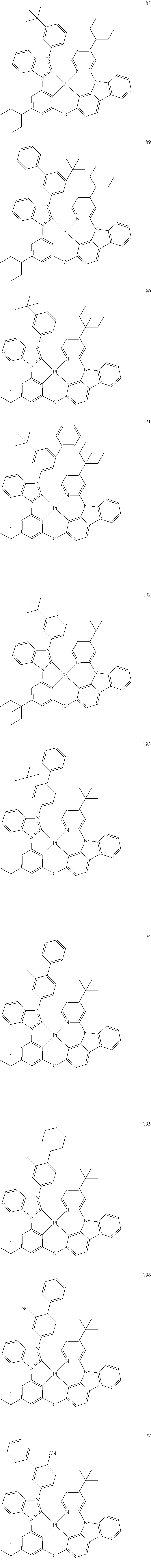

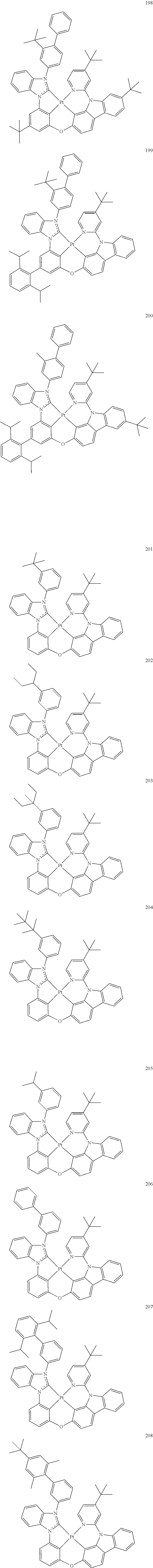

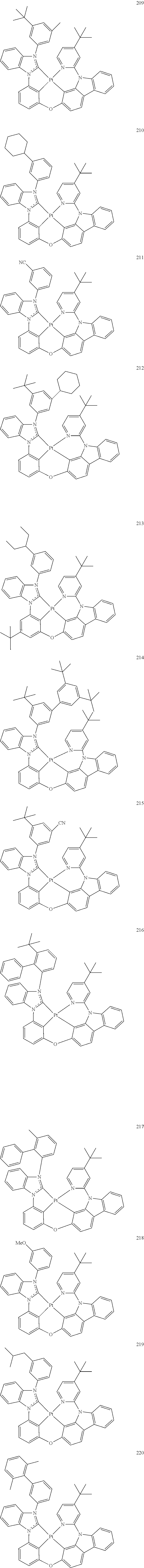

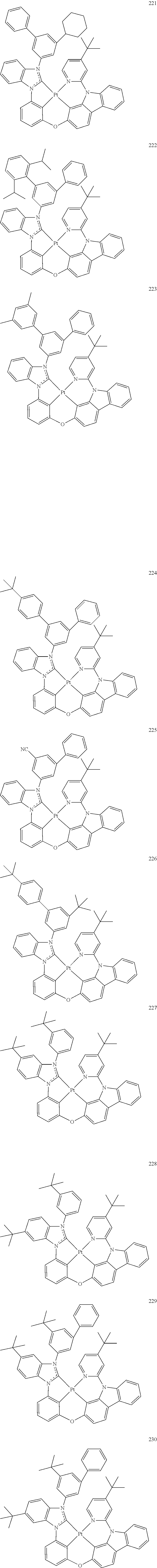

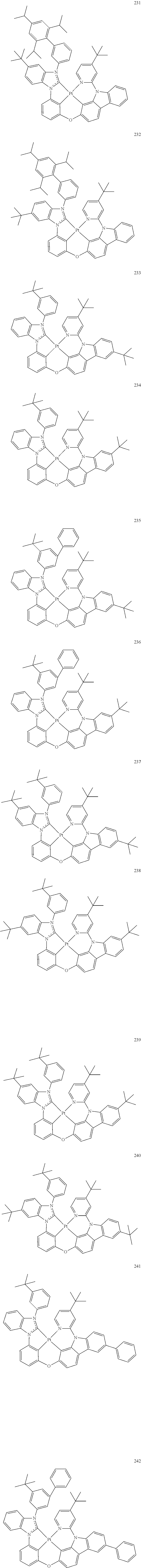

[chemical structure]

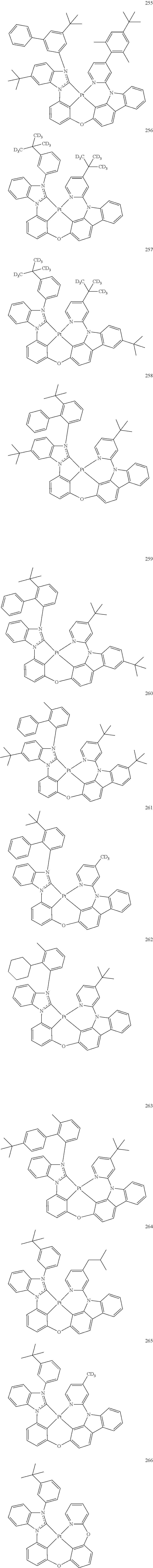

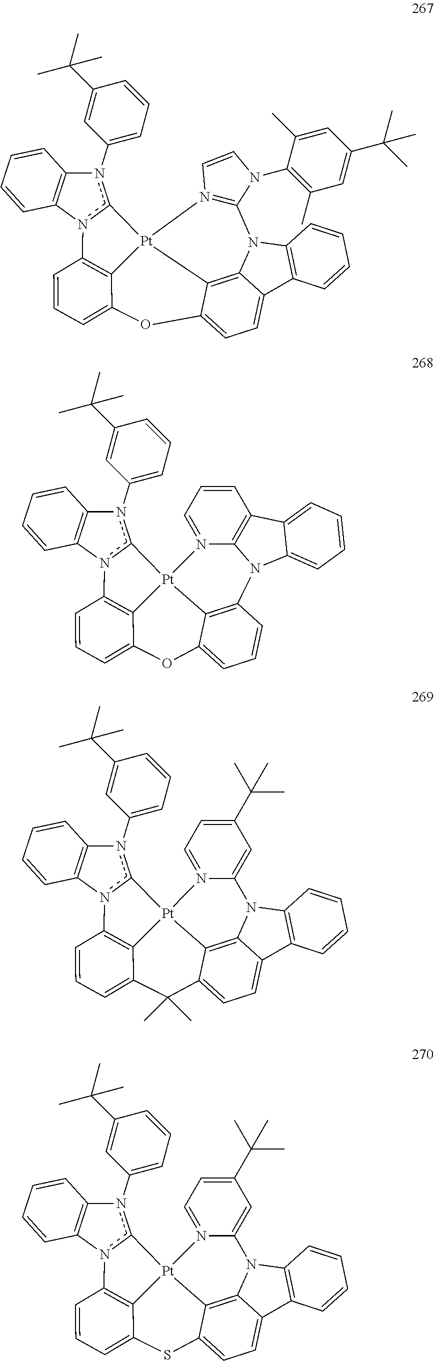

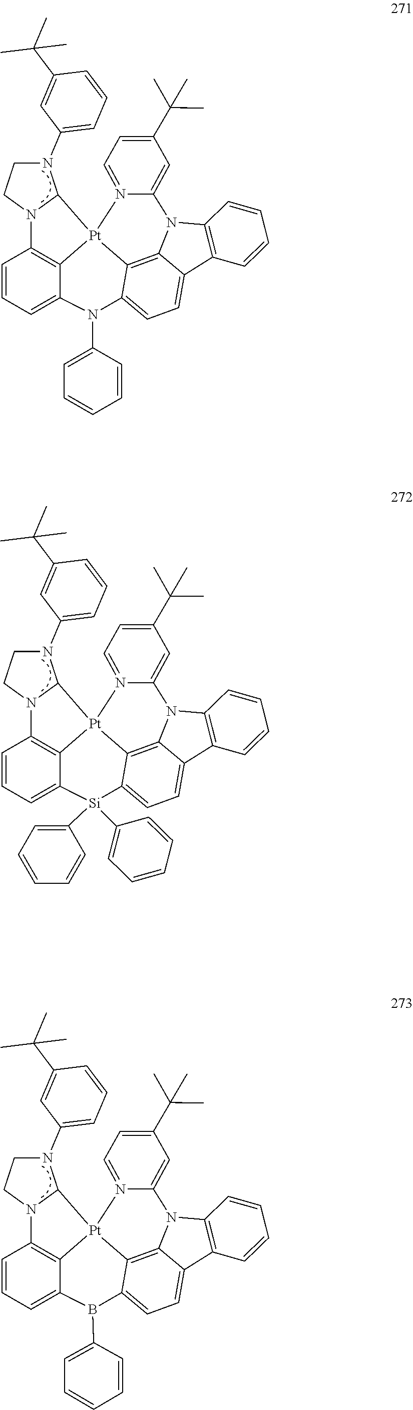

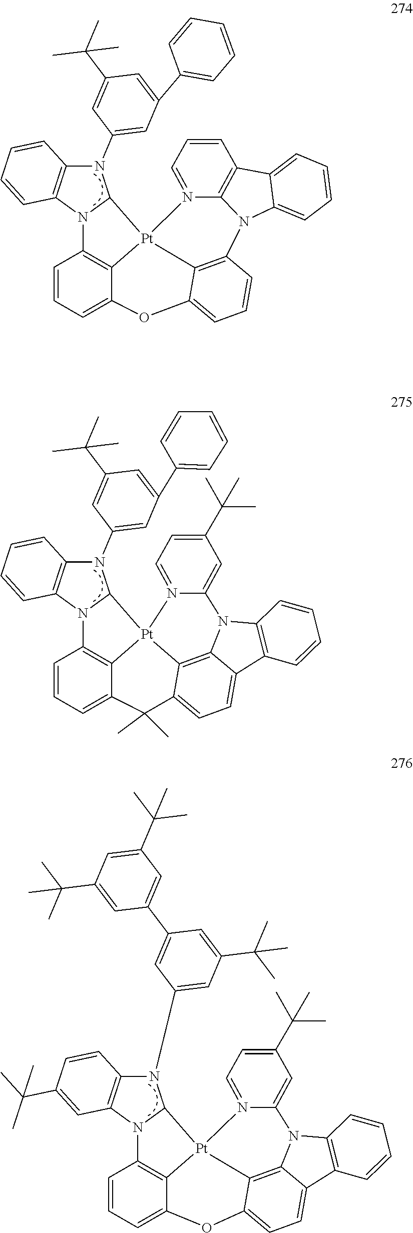

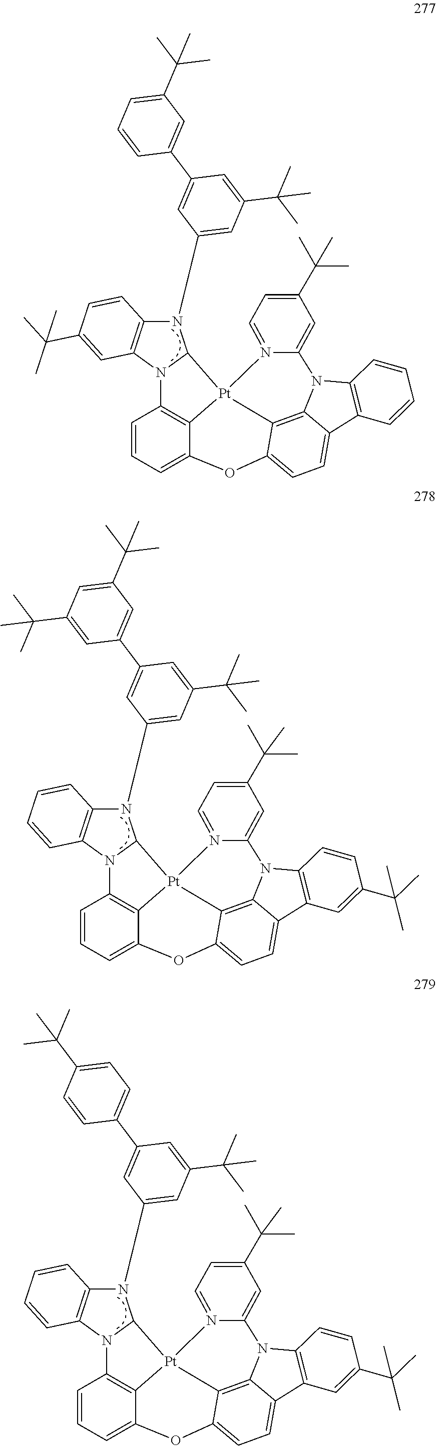

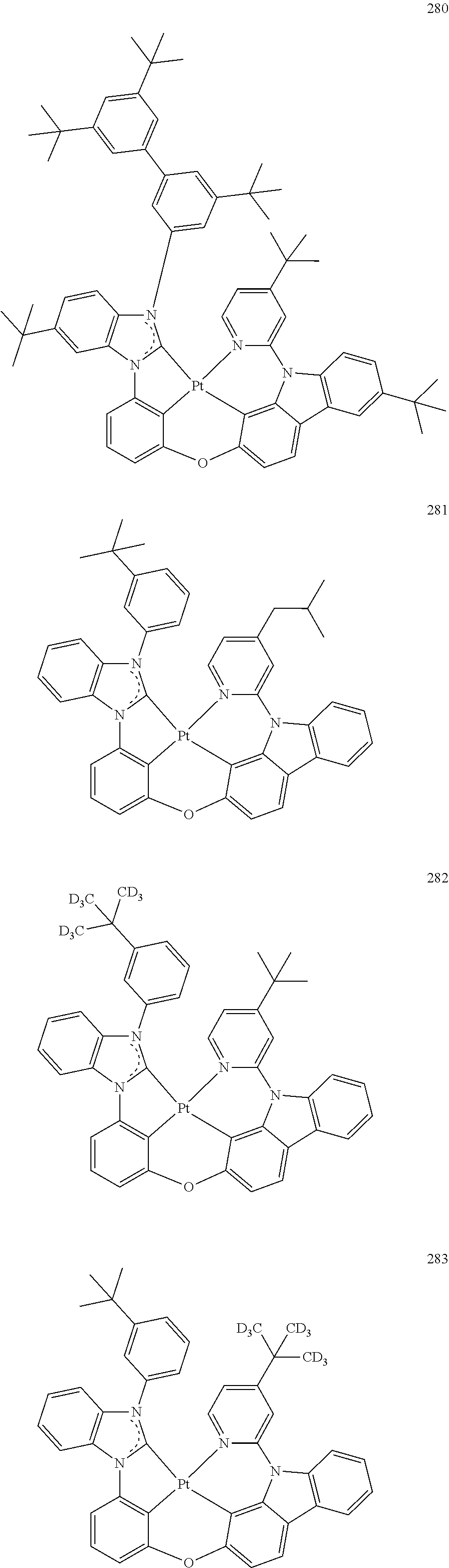

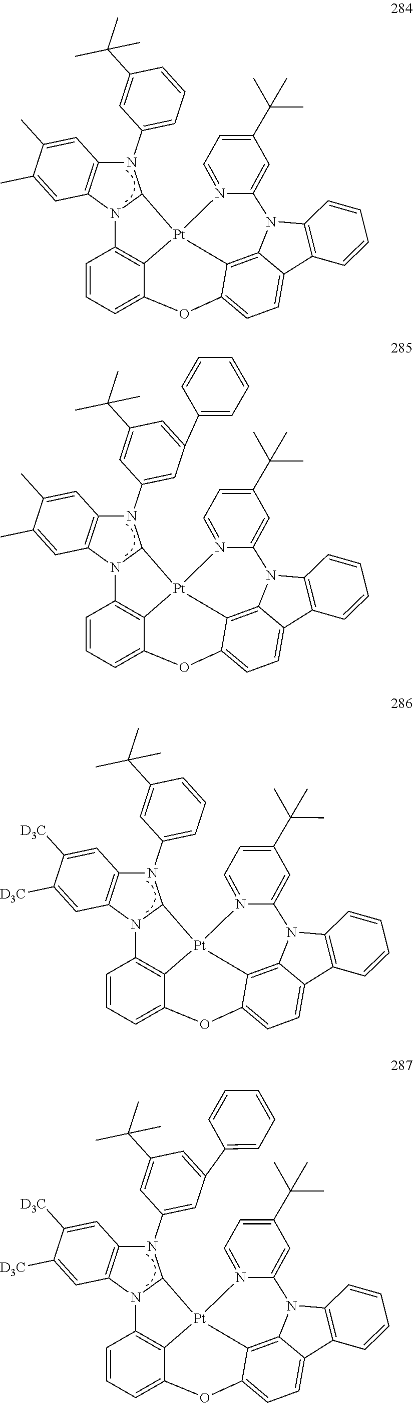

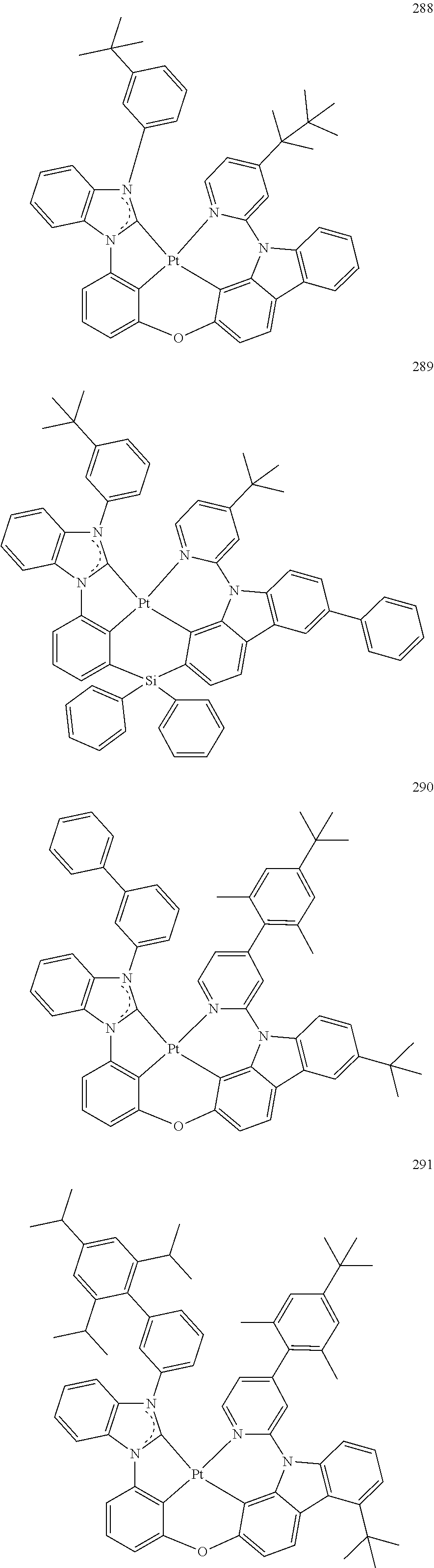

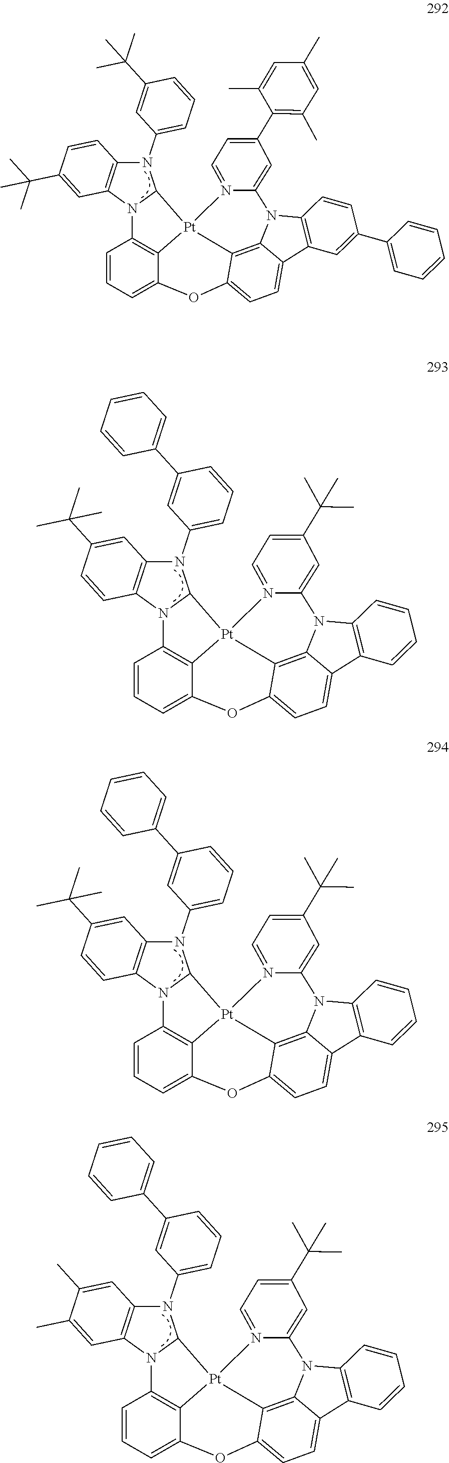

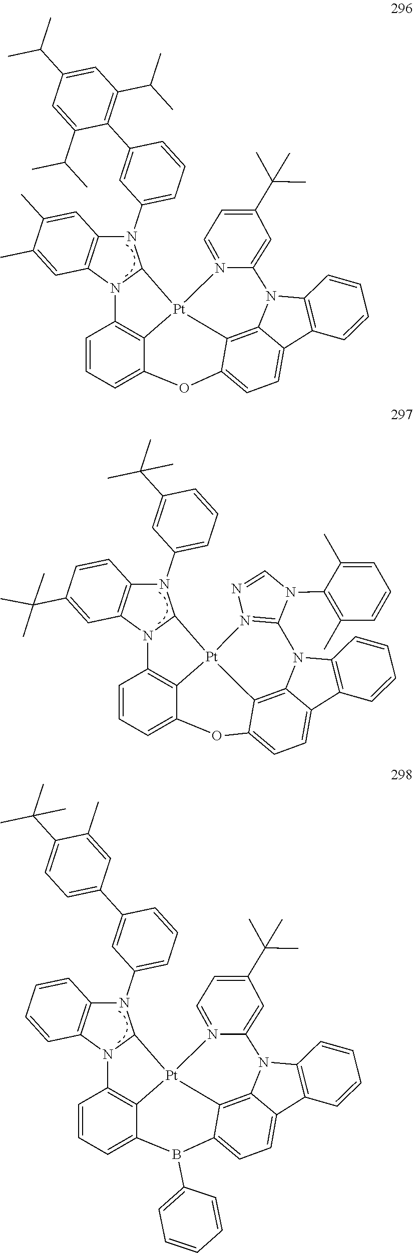

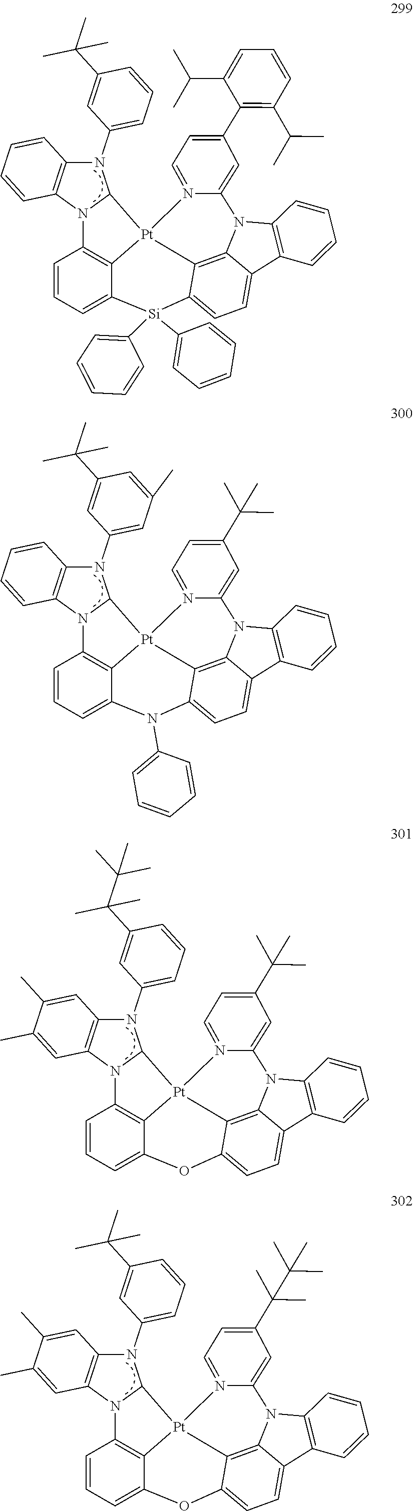

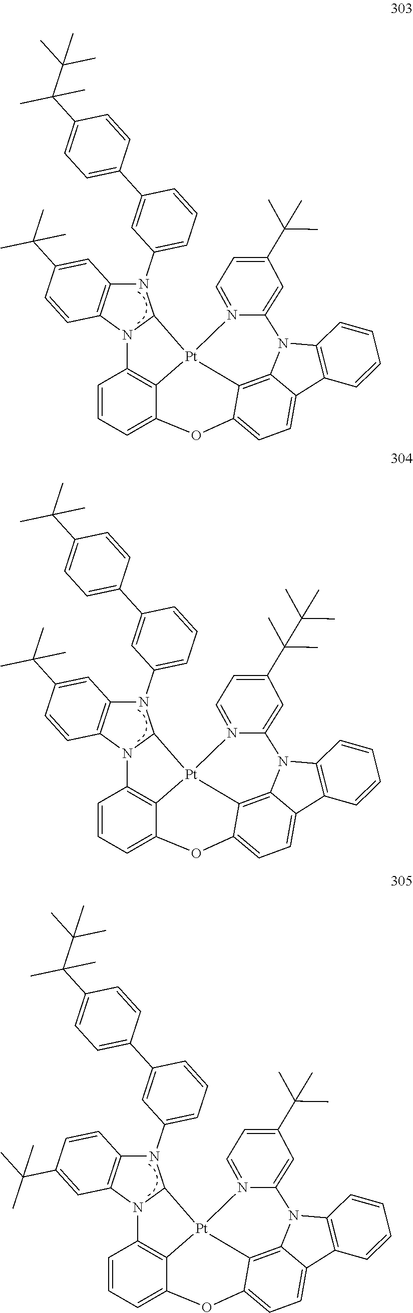

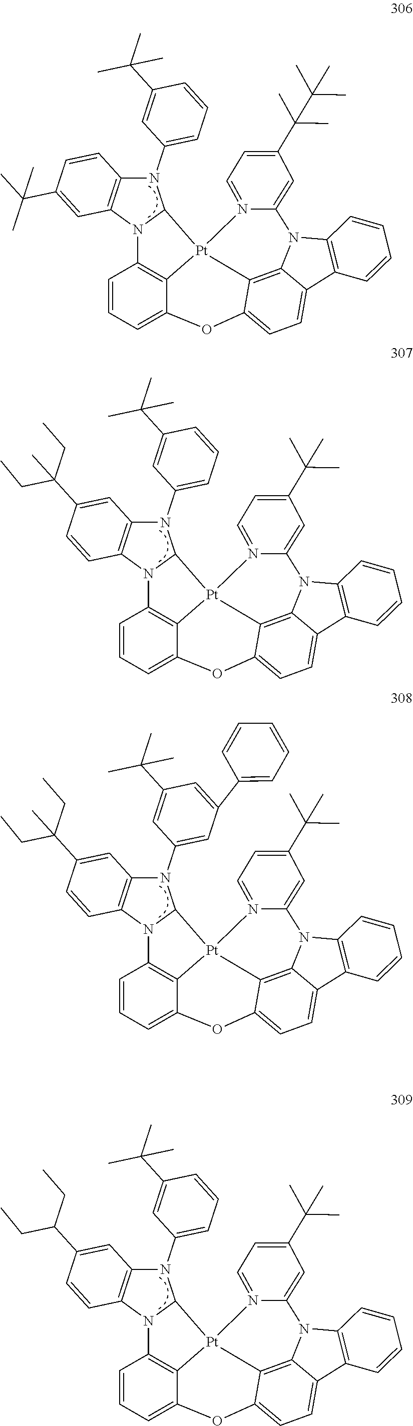

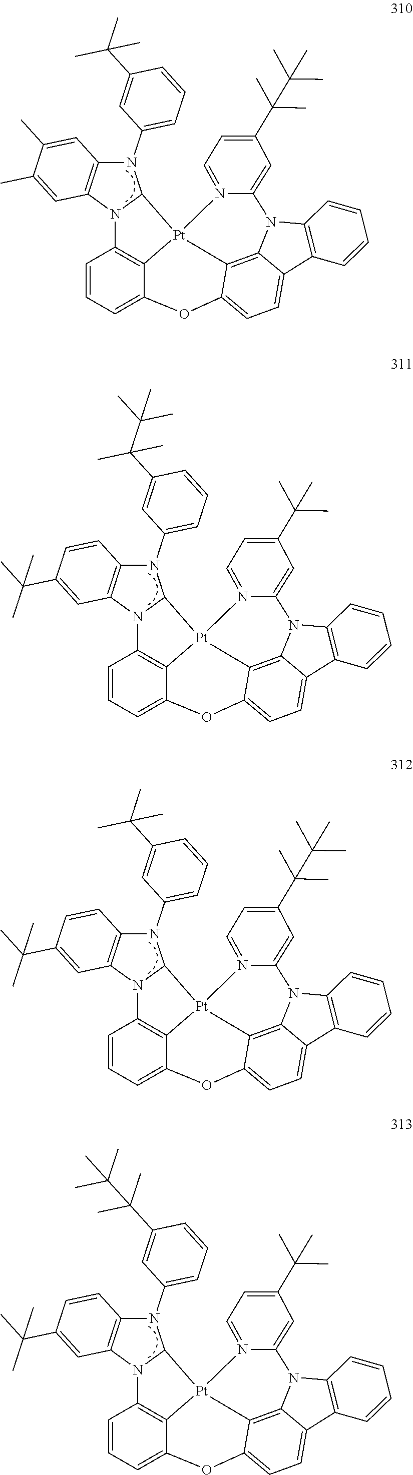

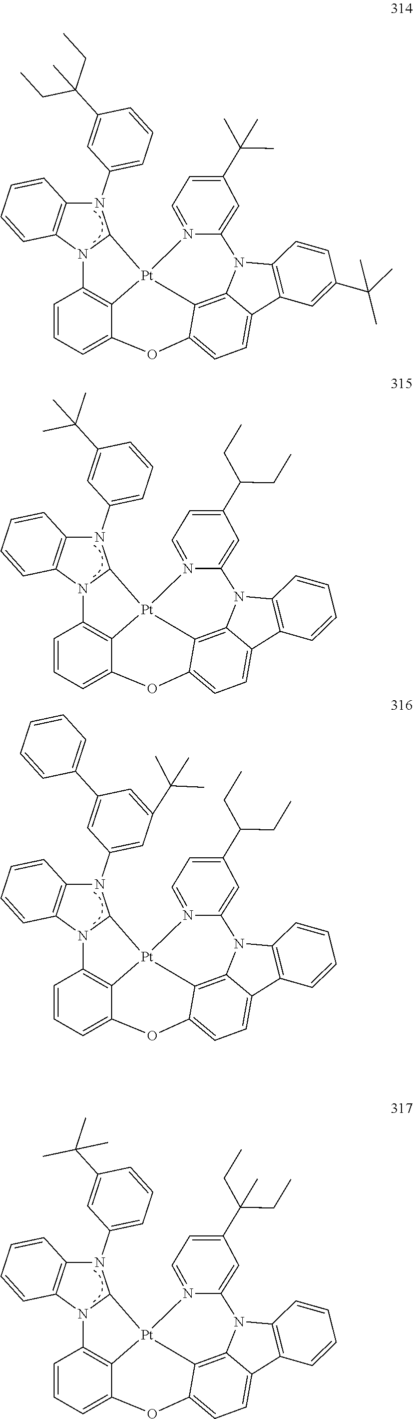

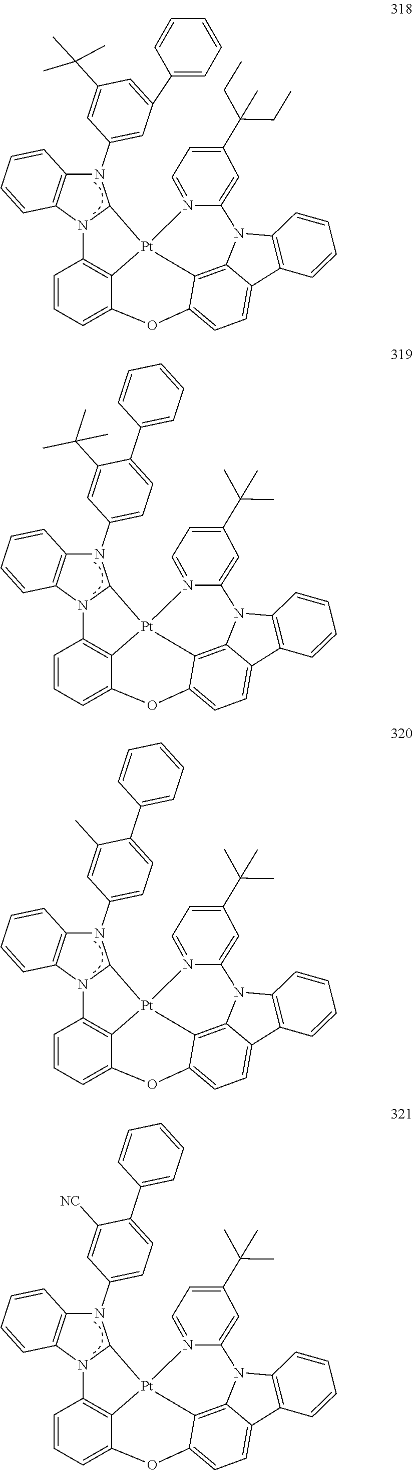

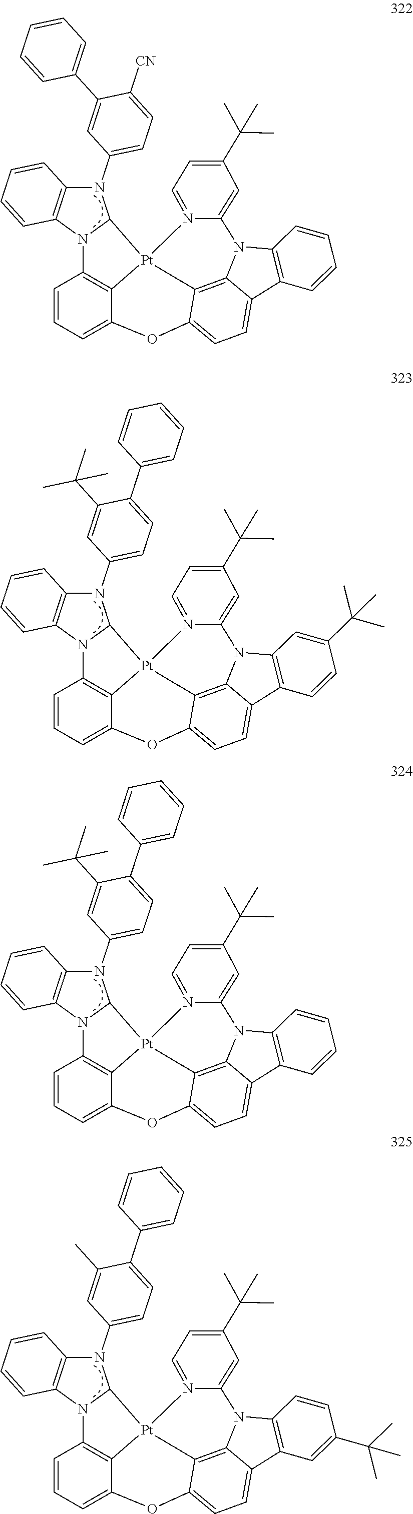

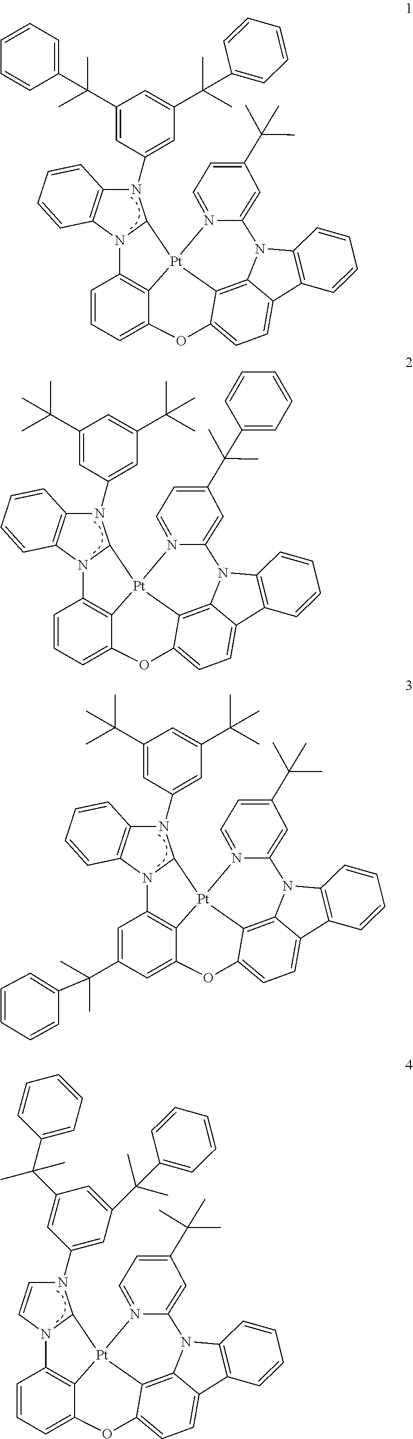

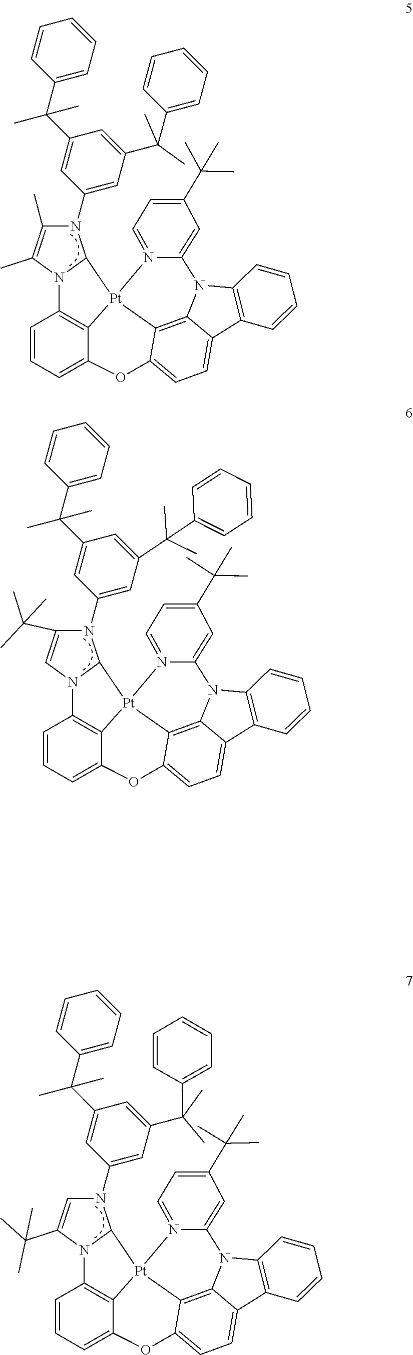

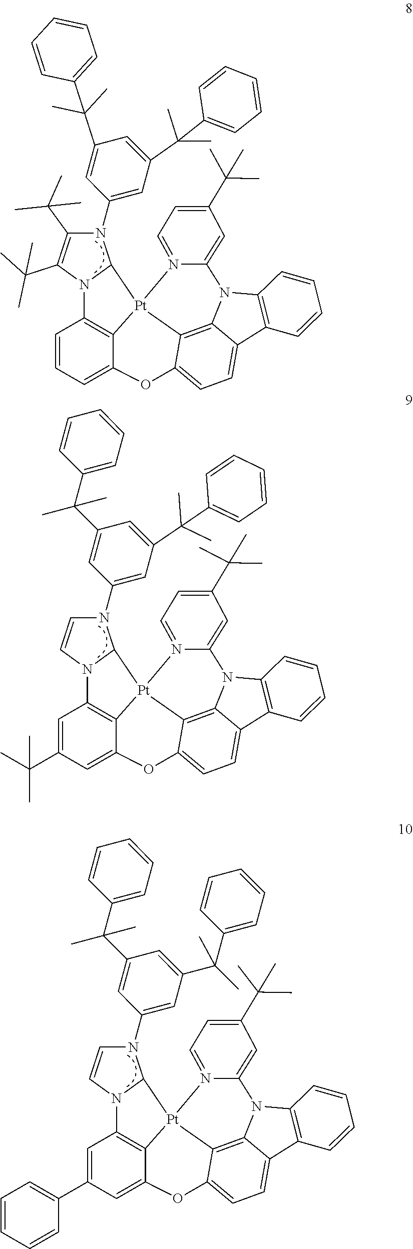

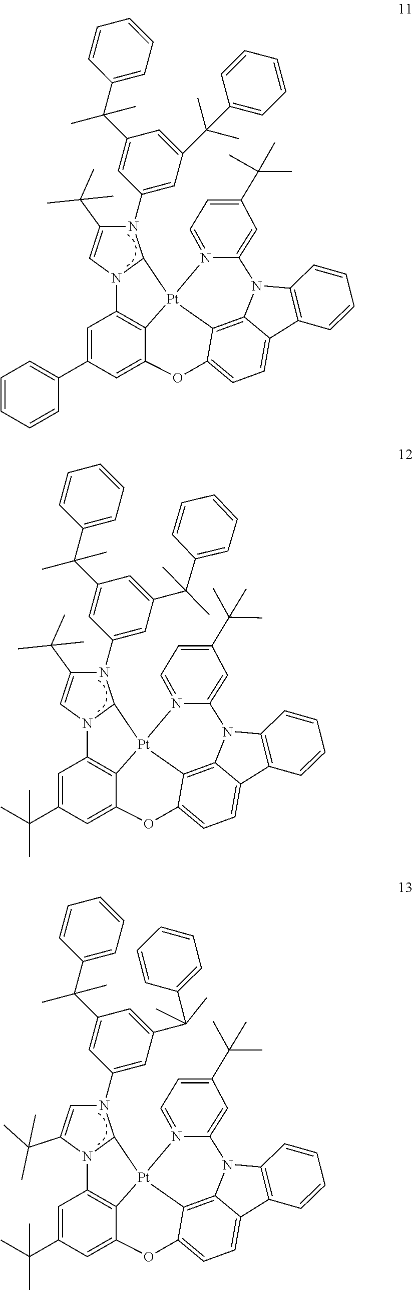

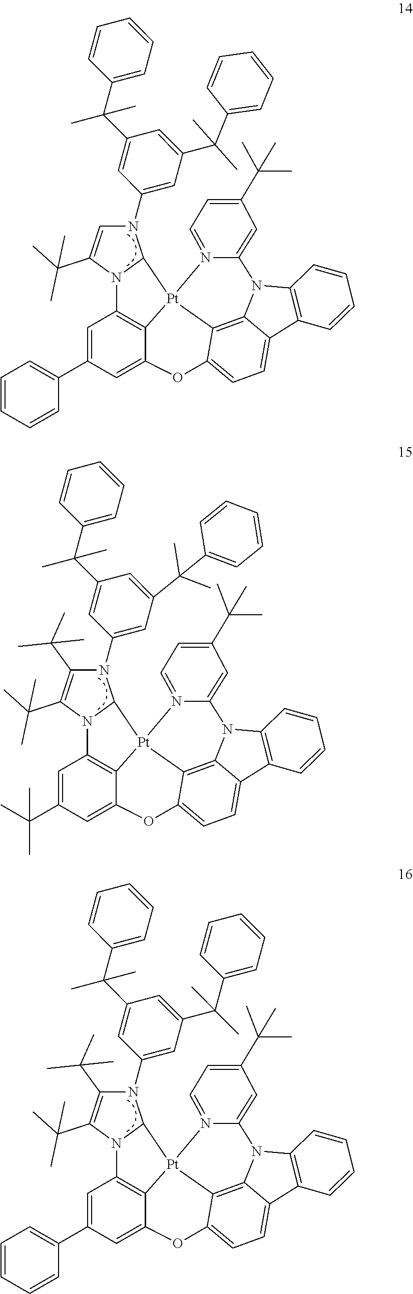

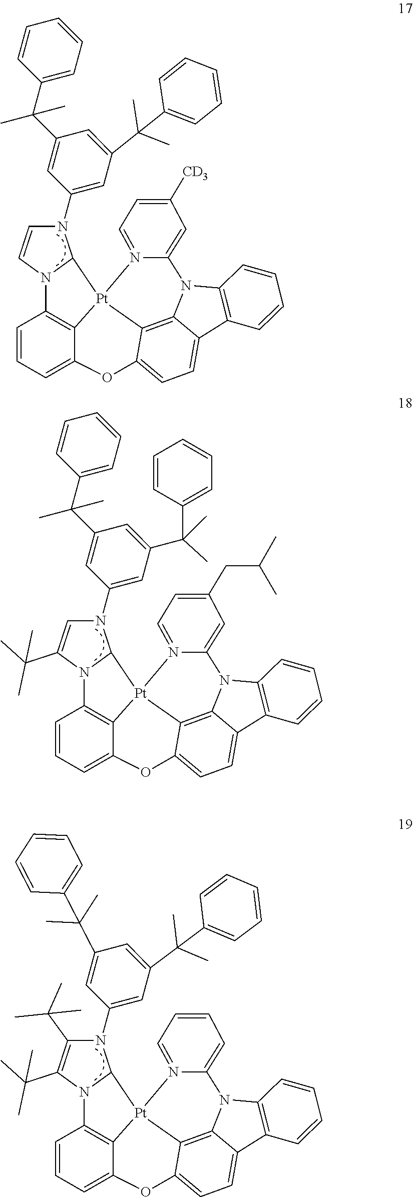

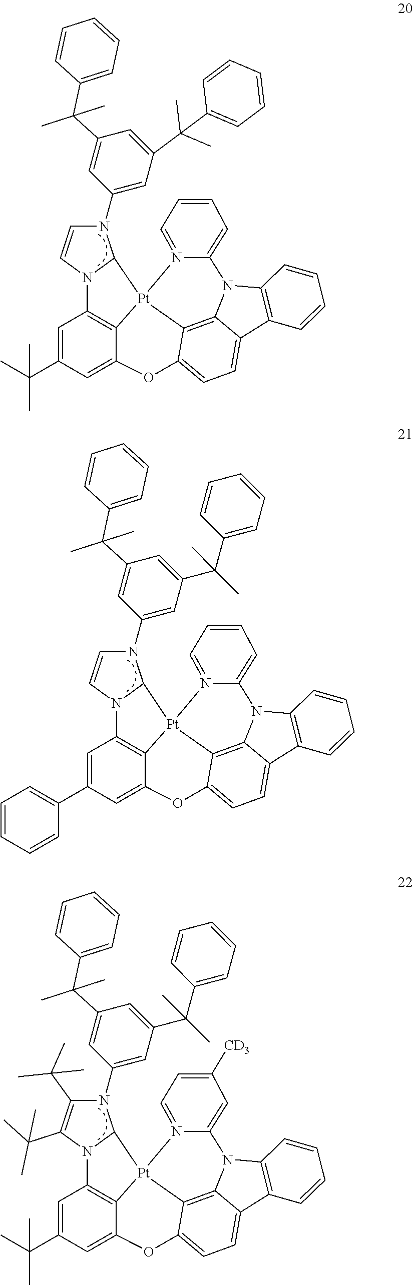

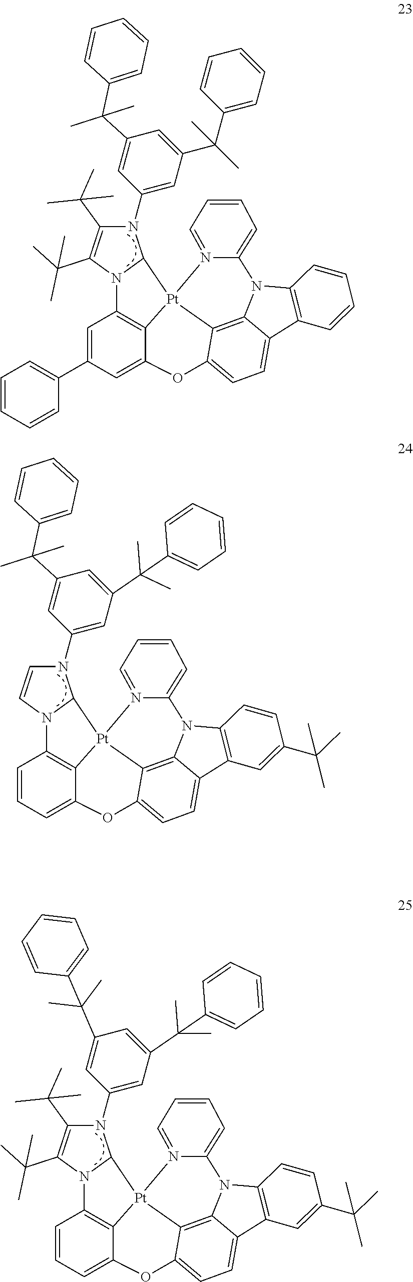

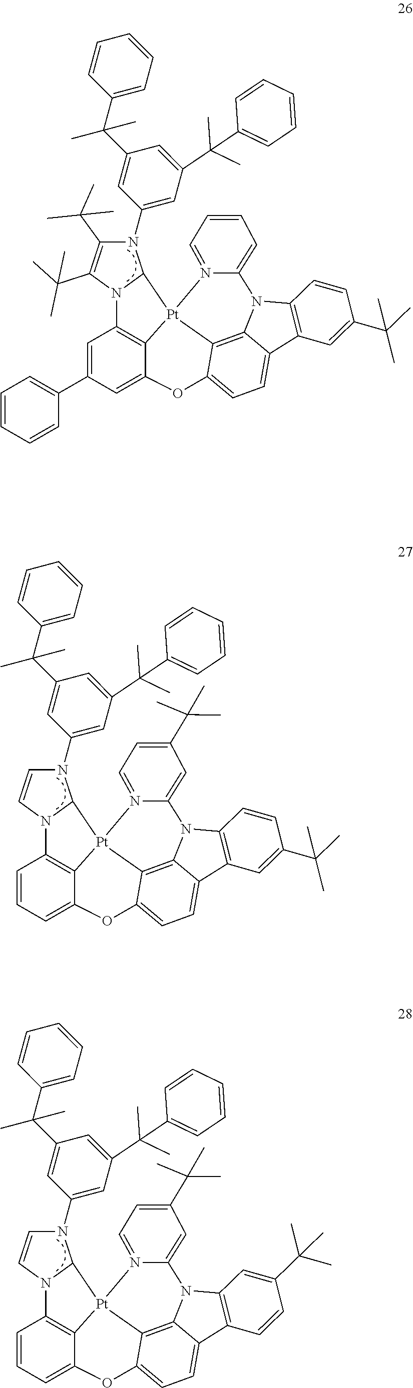

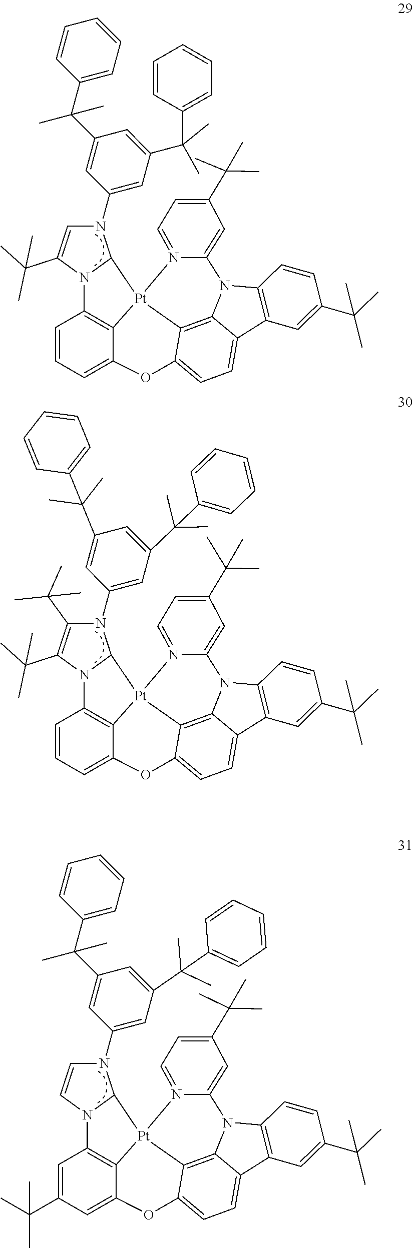

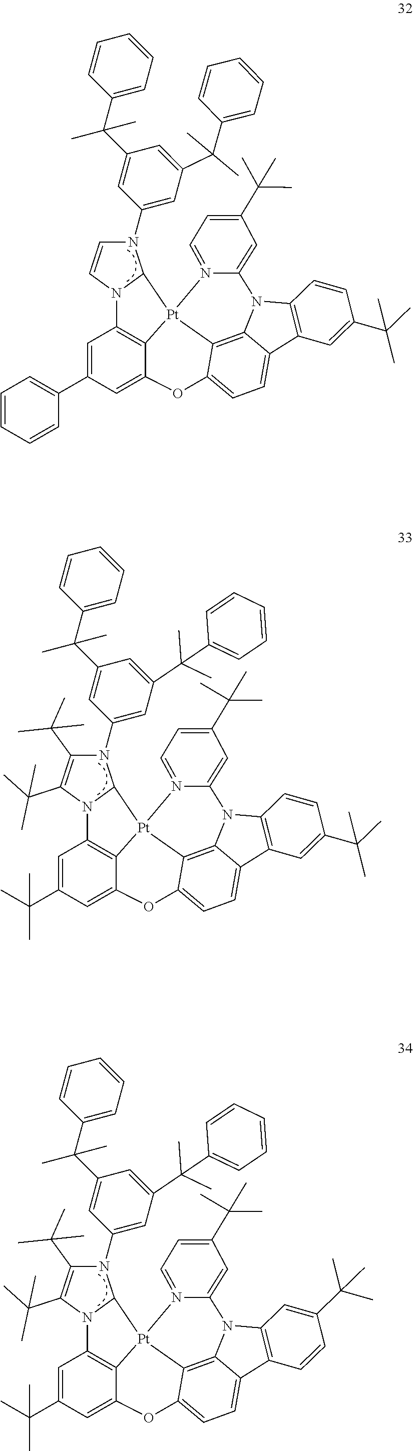

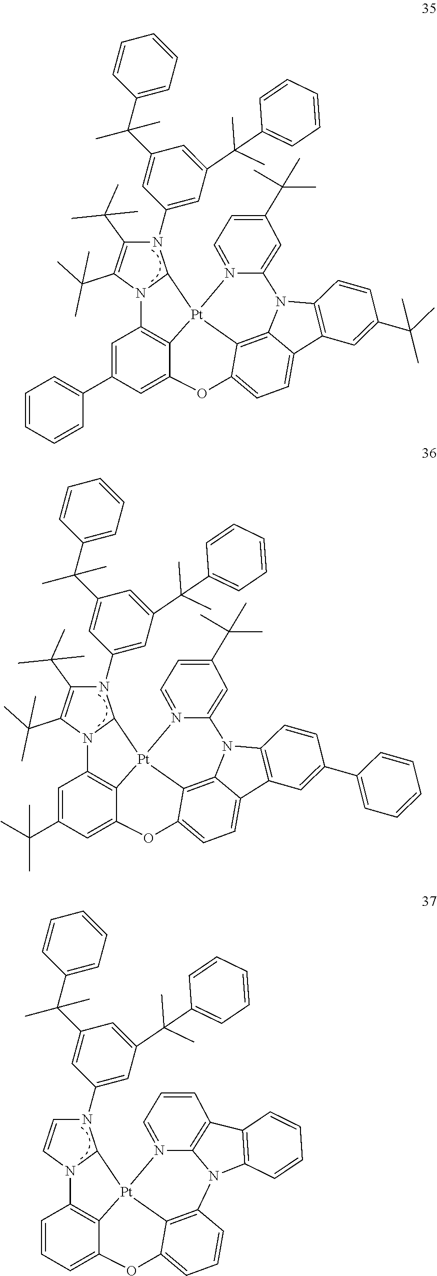

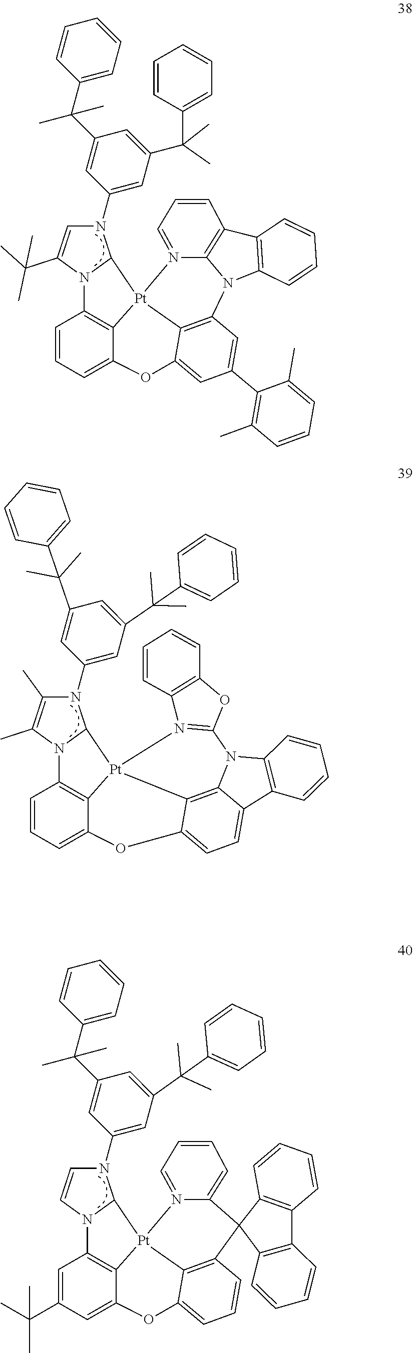

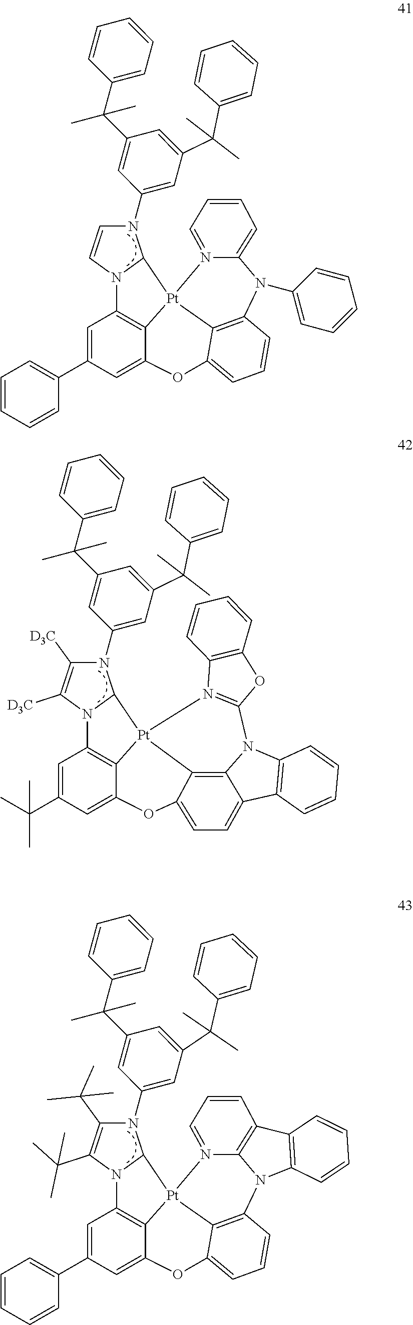

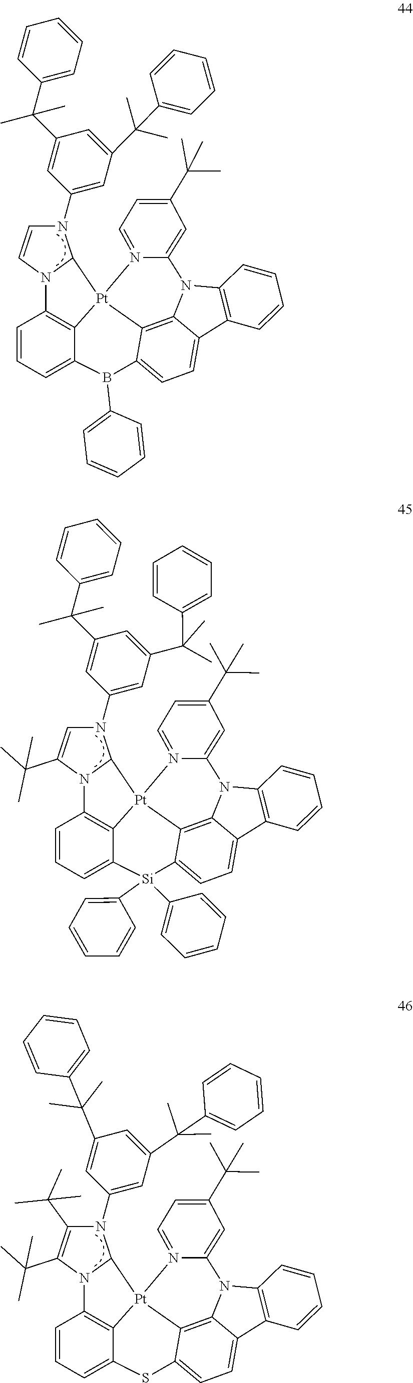

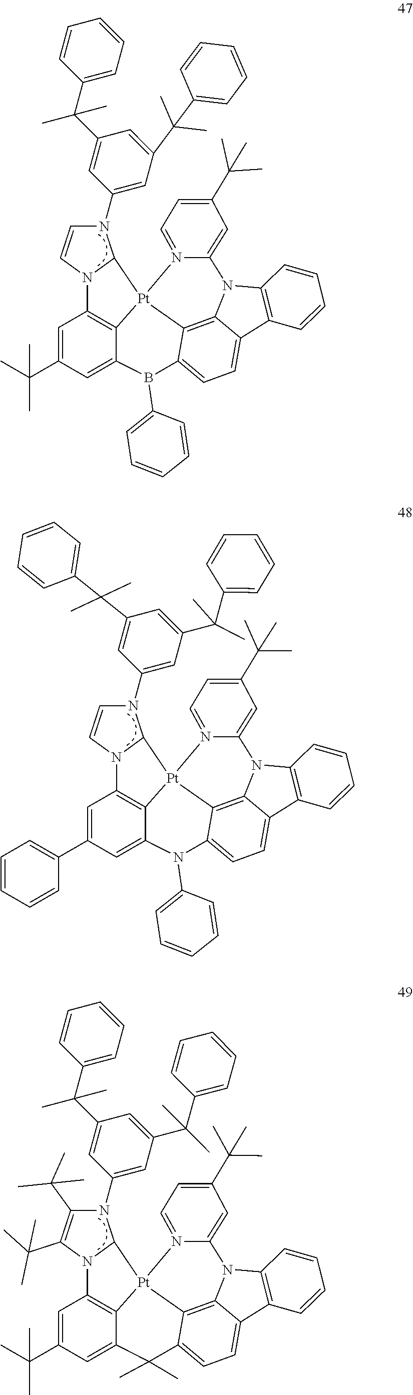

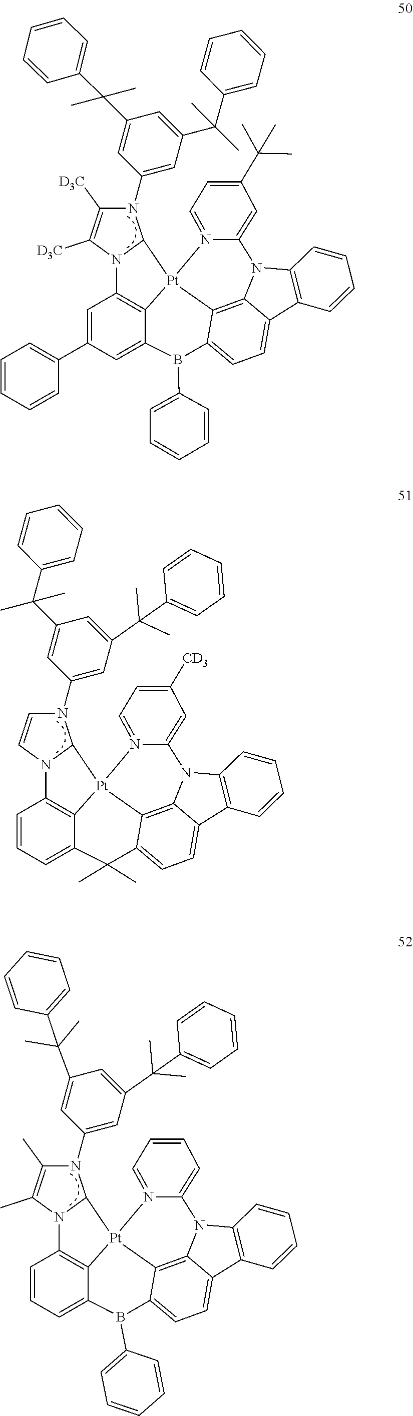

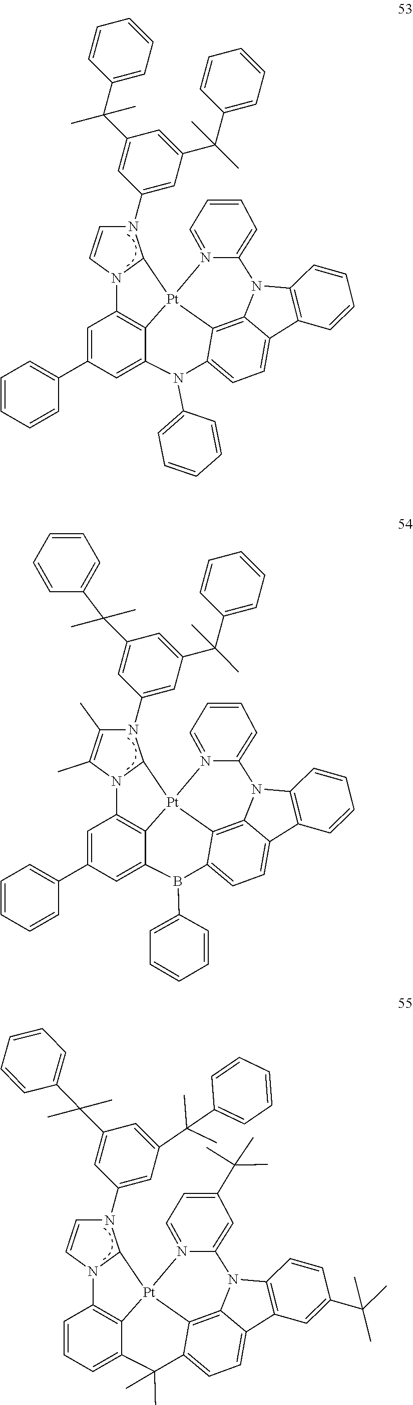

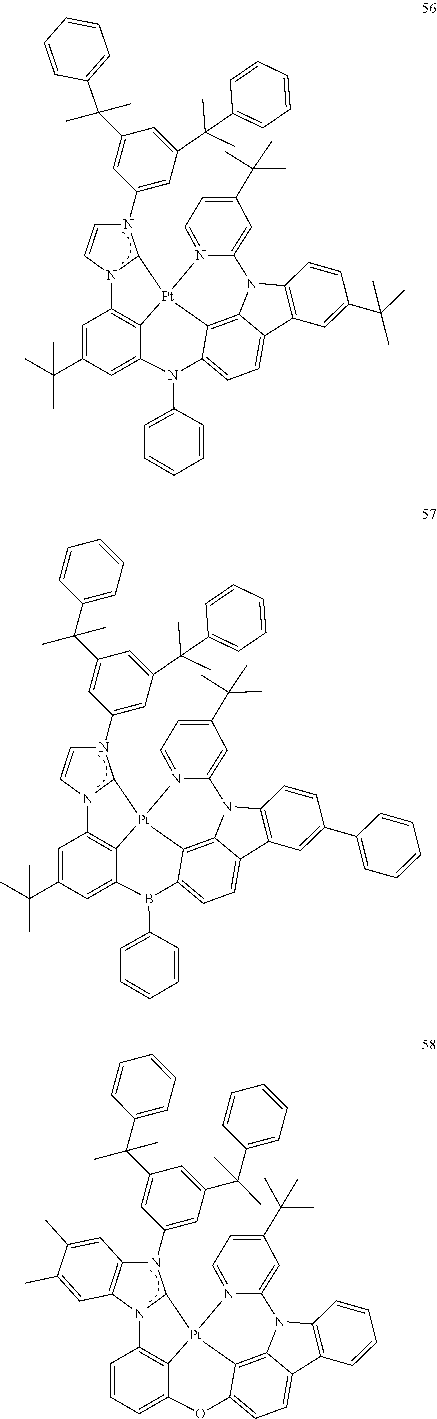

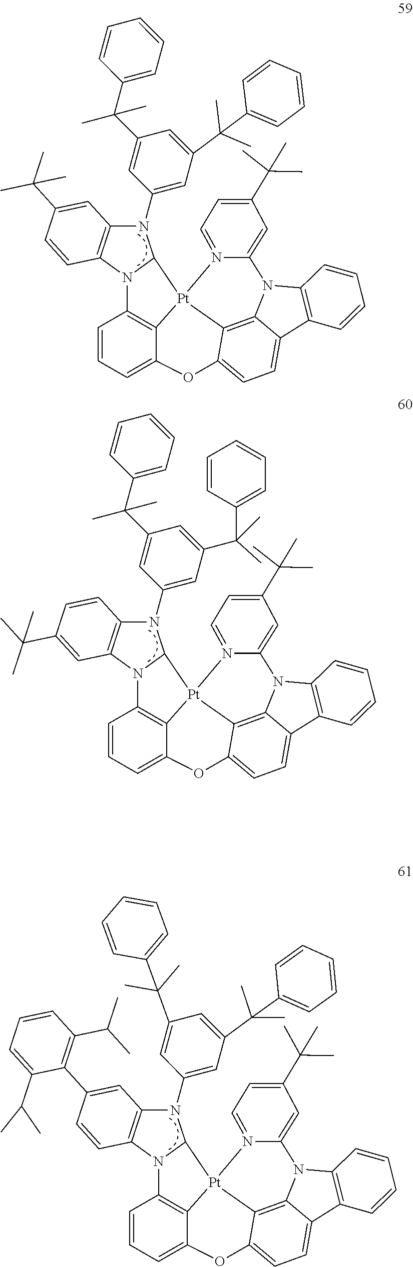

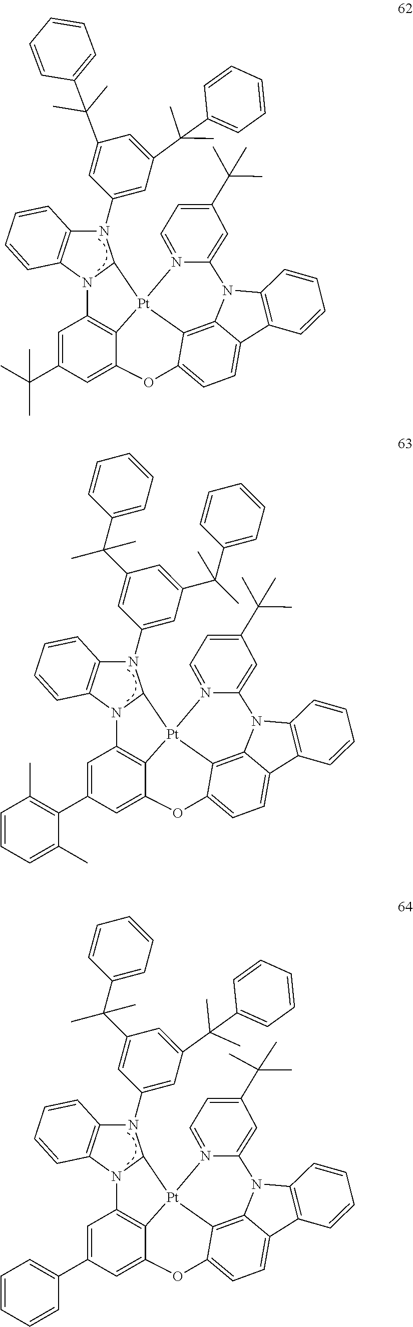

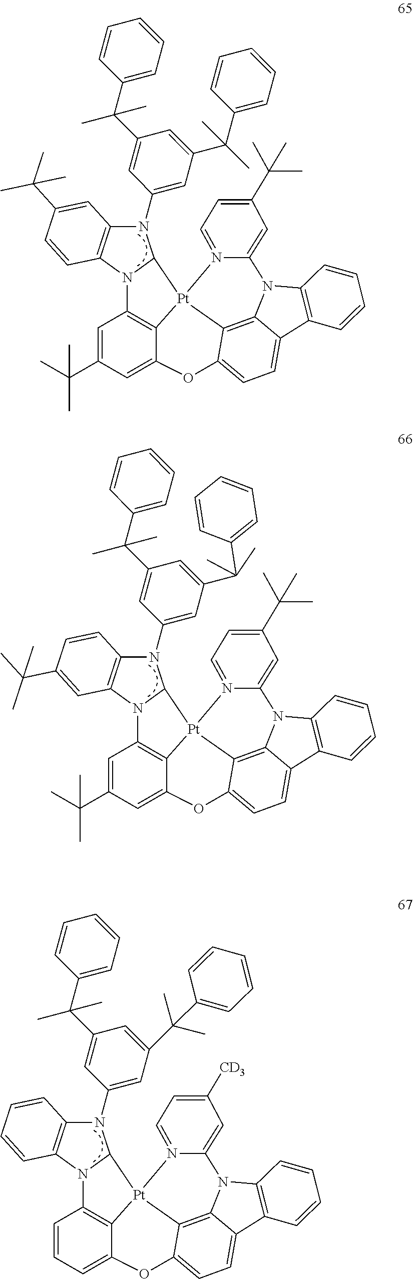

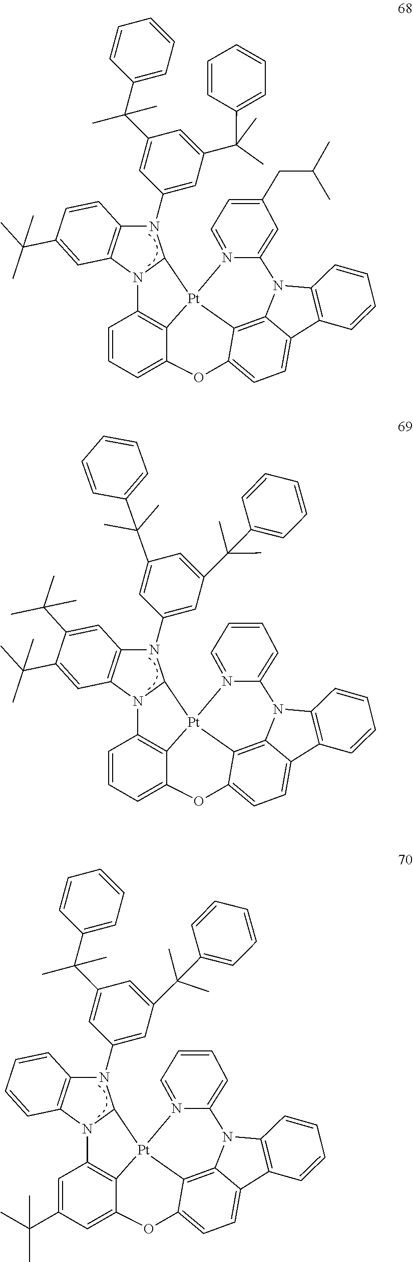

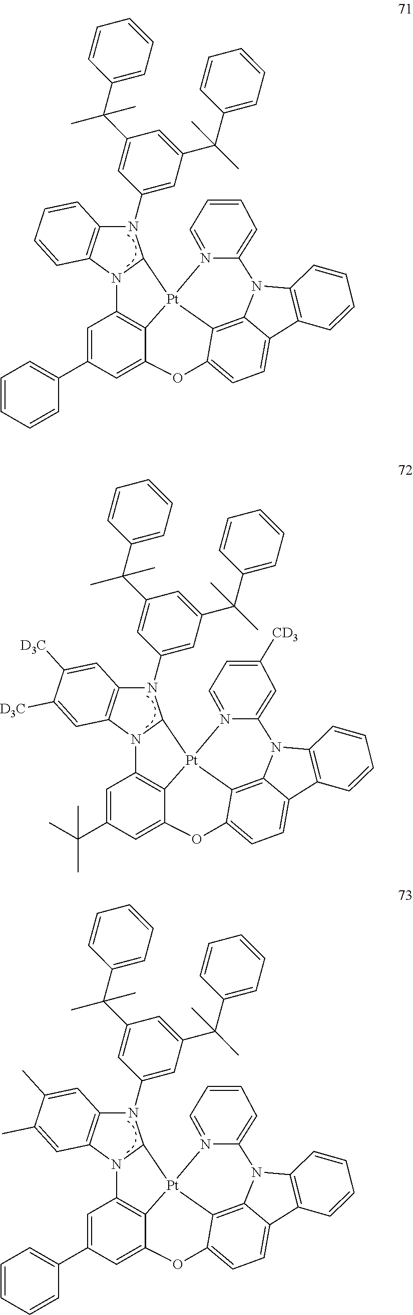

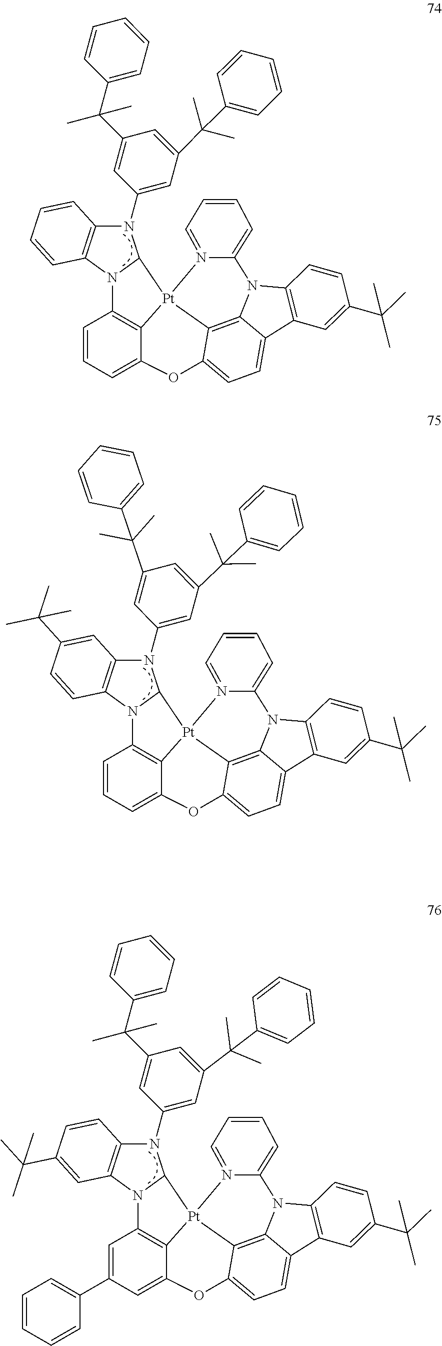

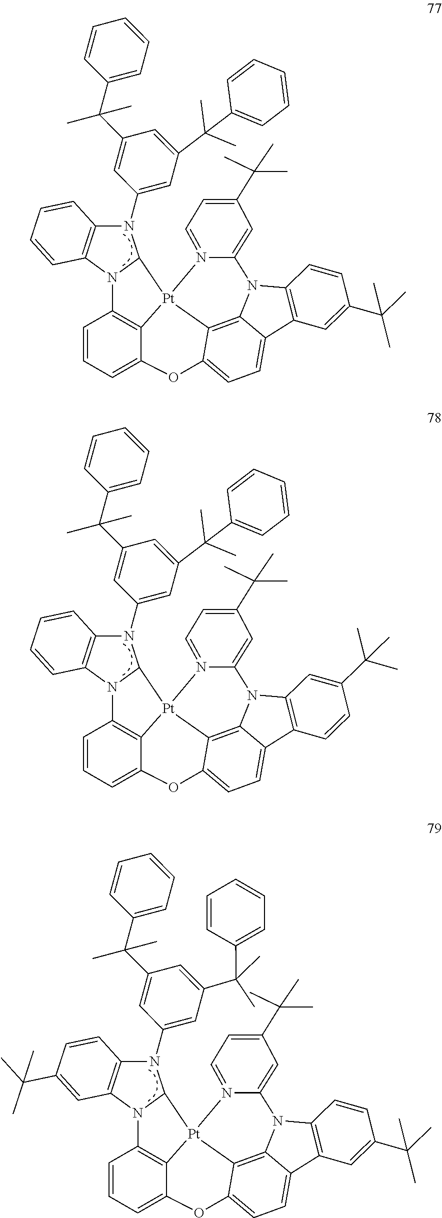

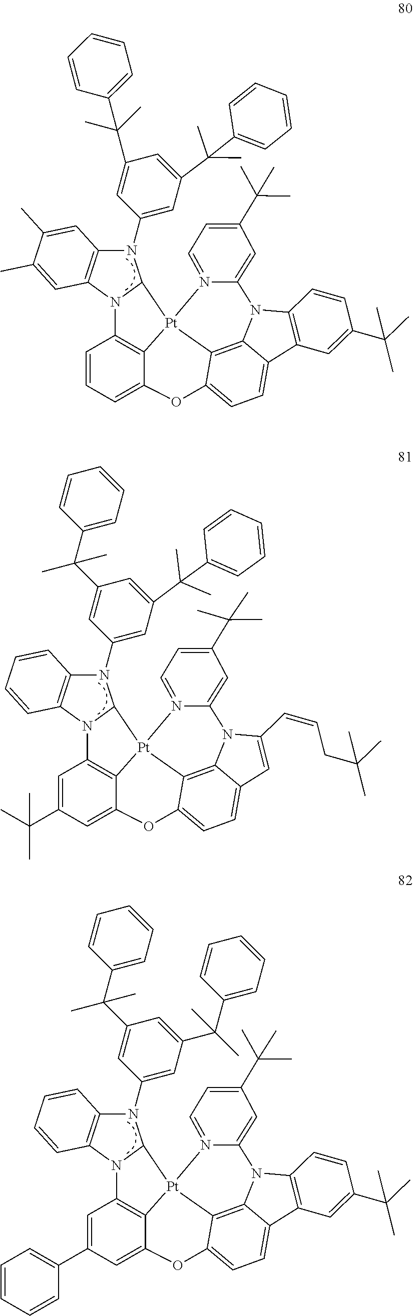

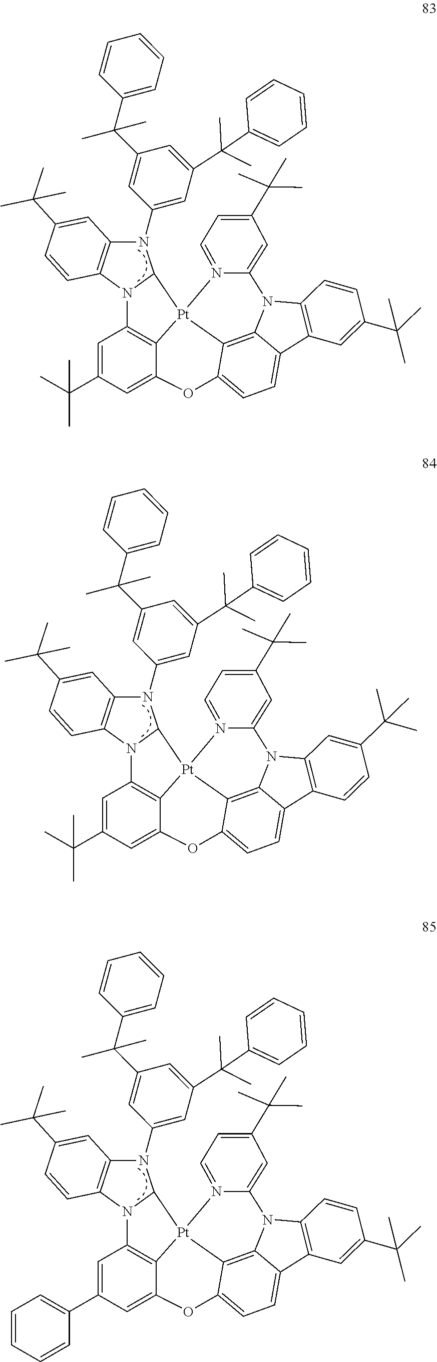

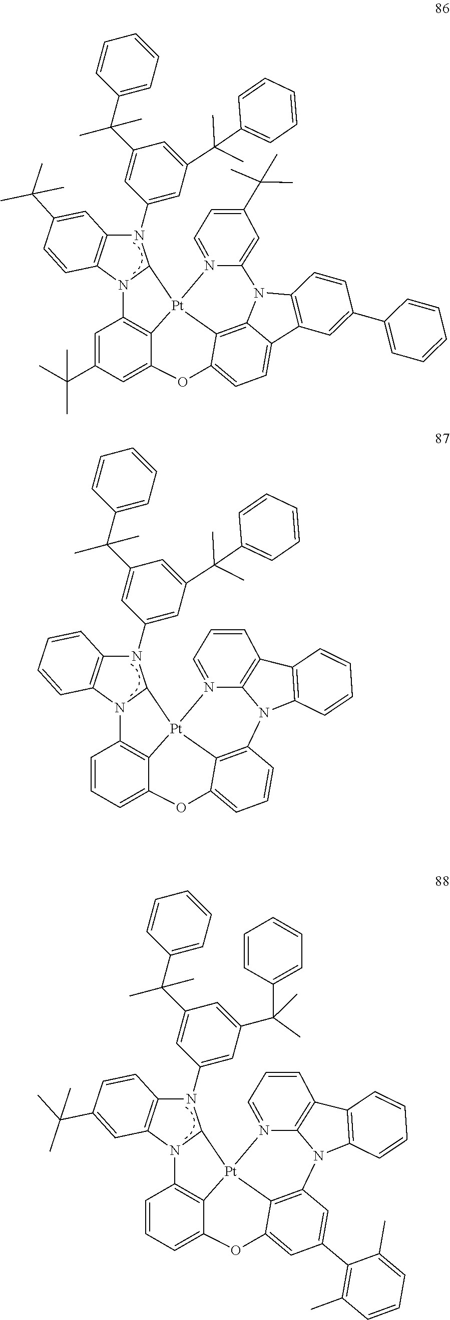

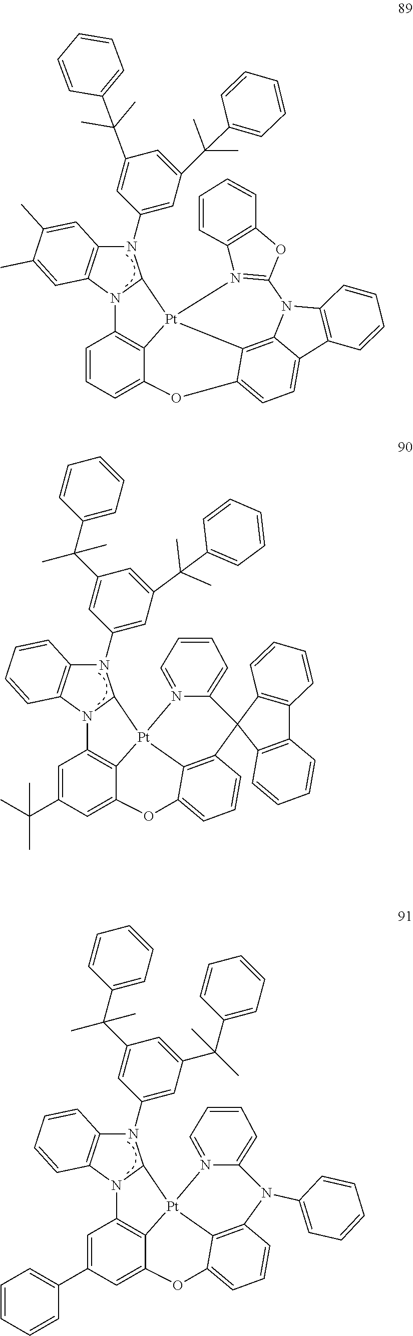

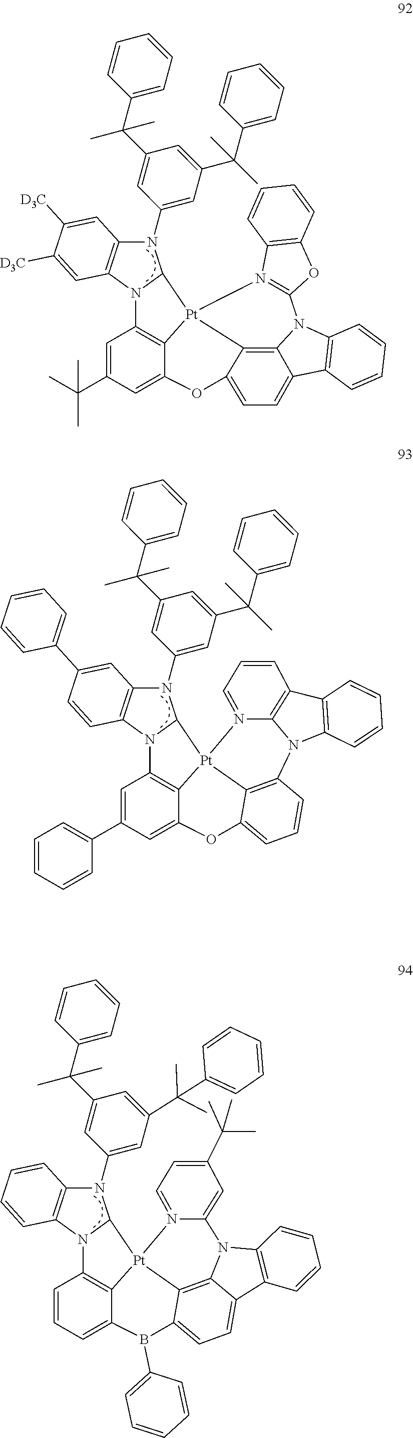

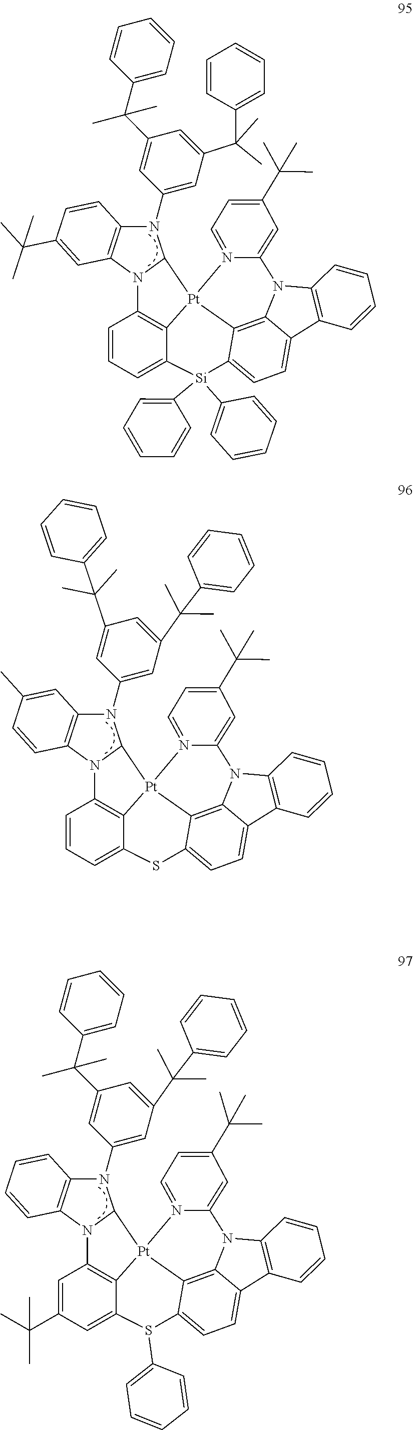

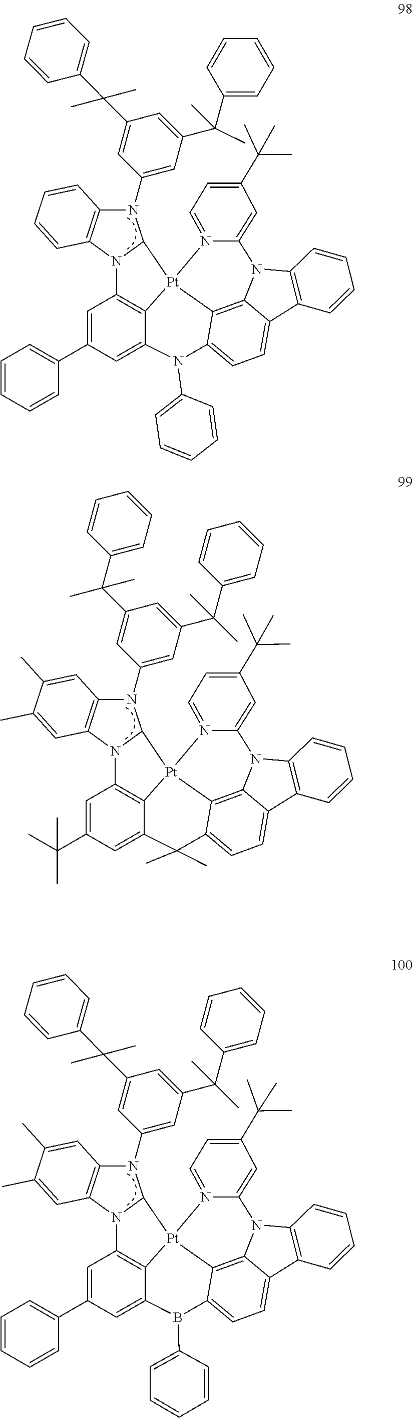

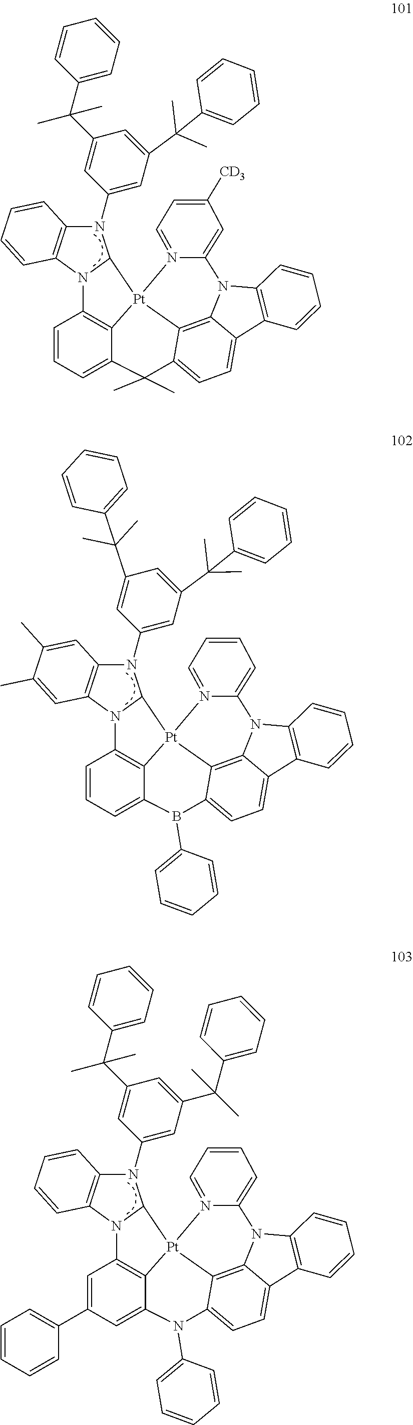

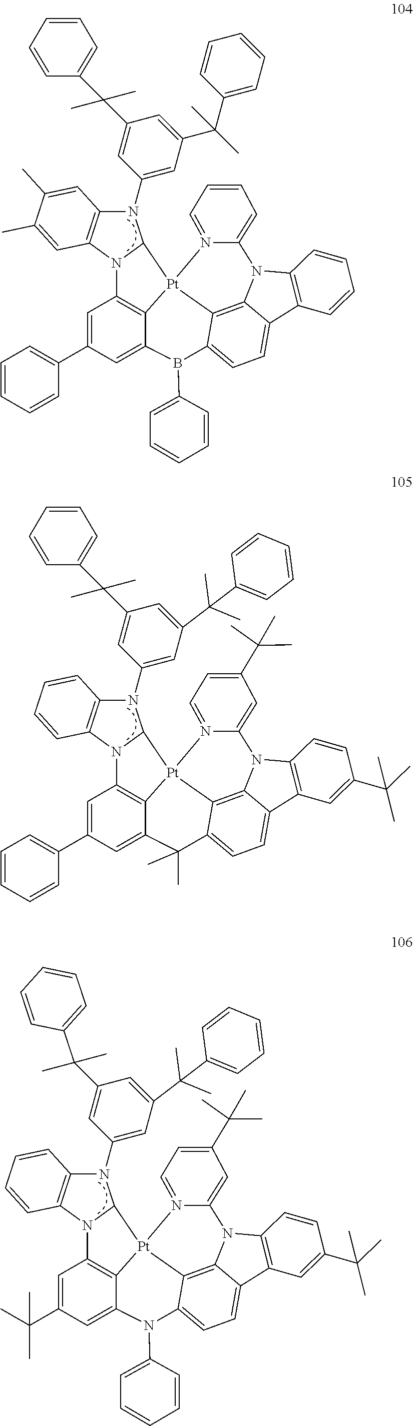

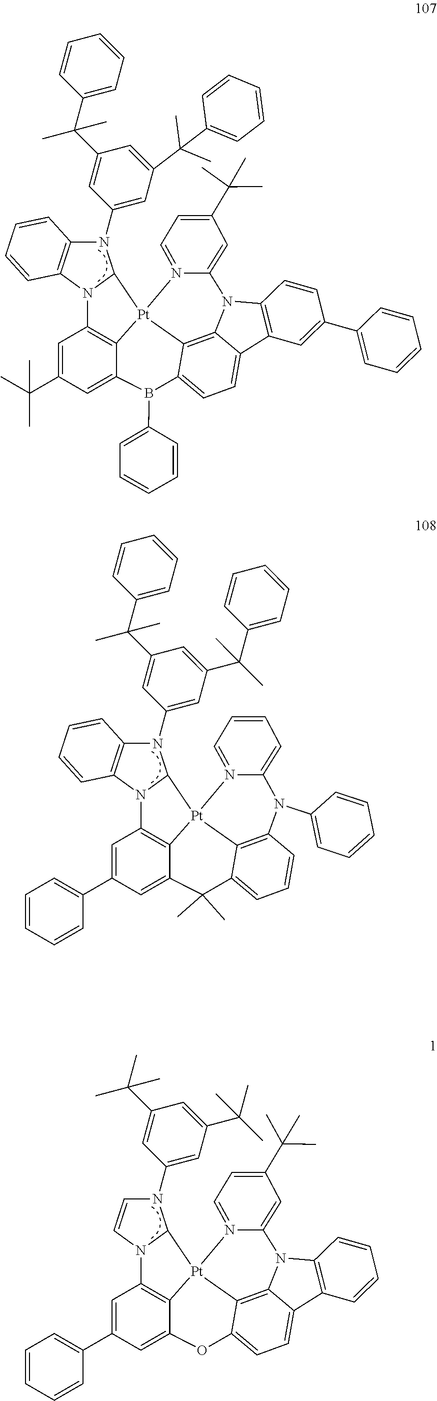

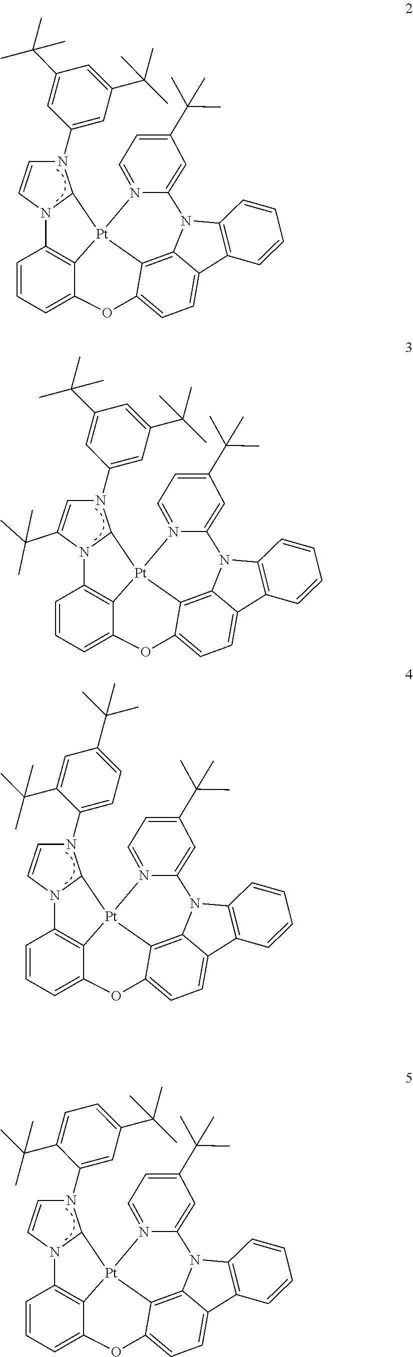

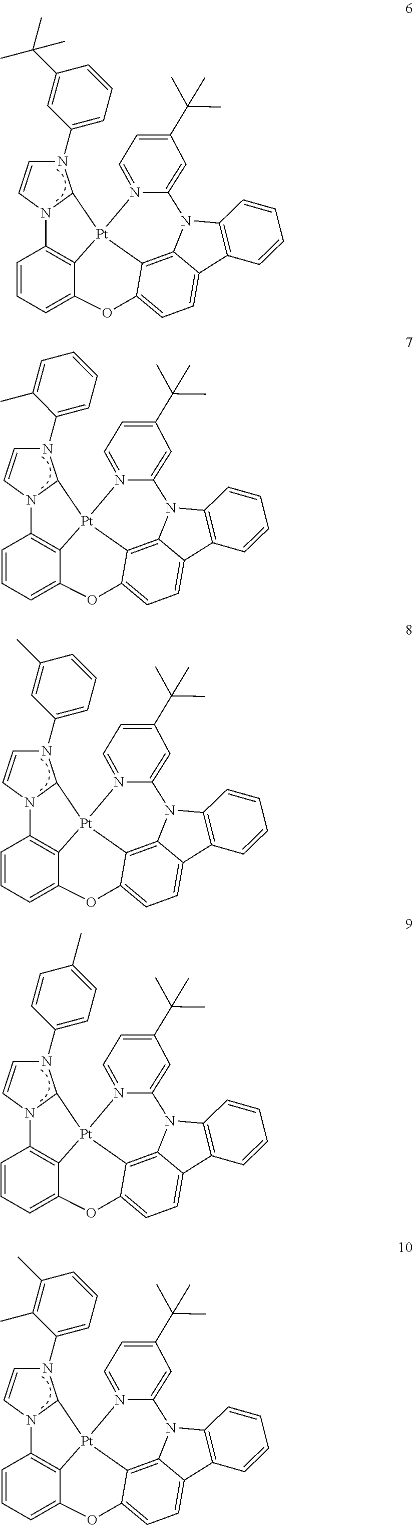

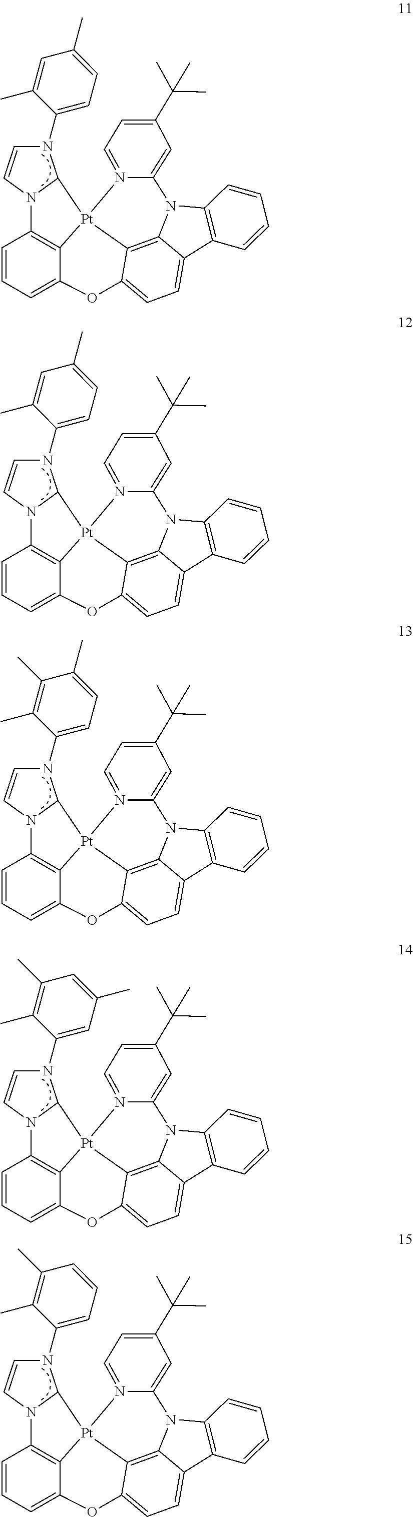

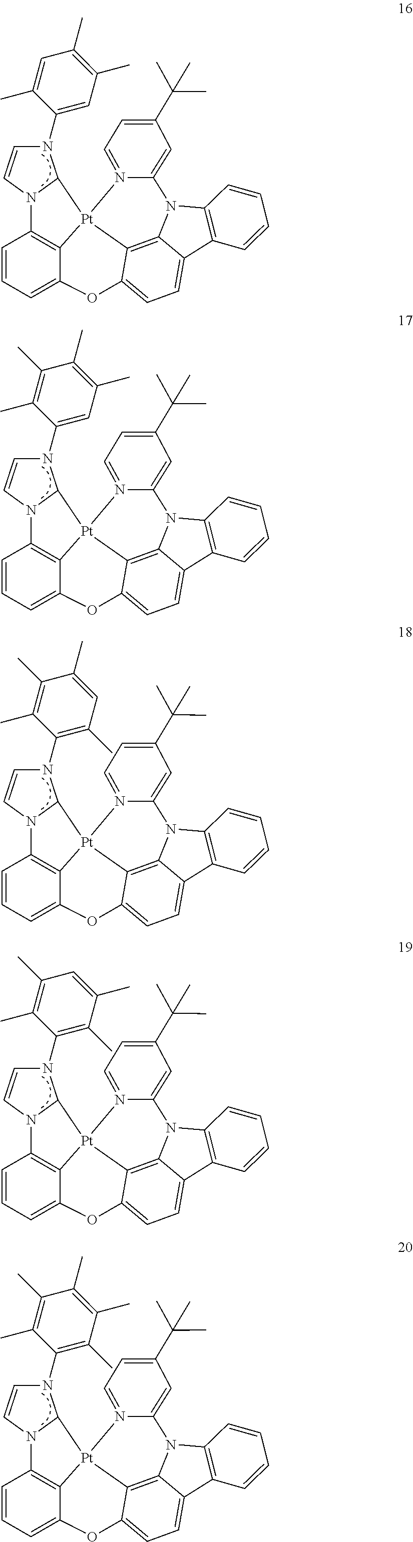

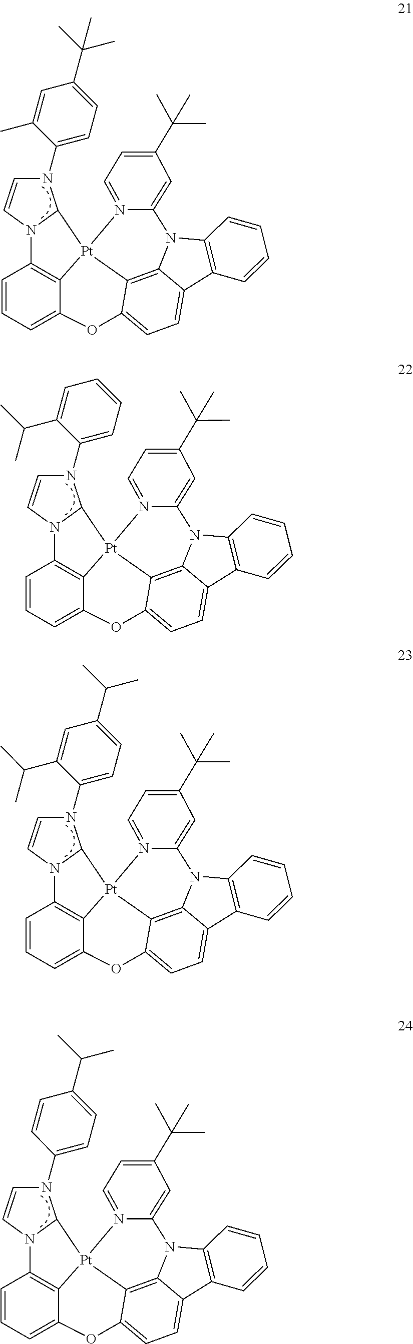

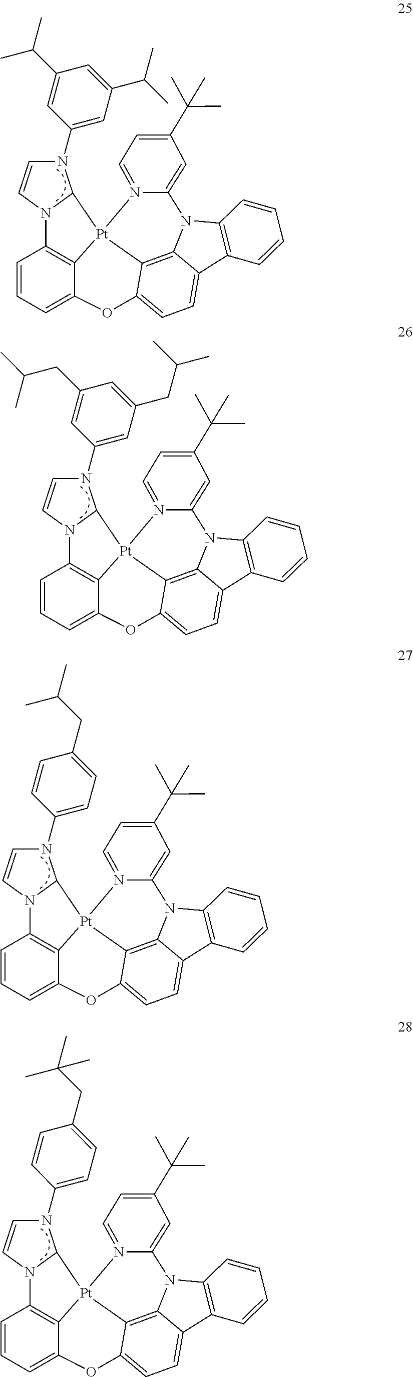

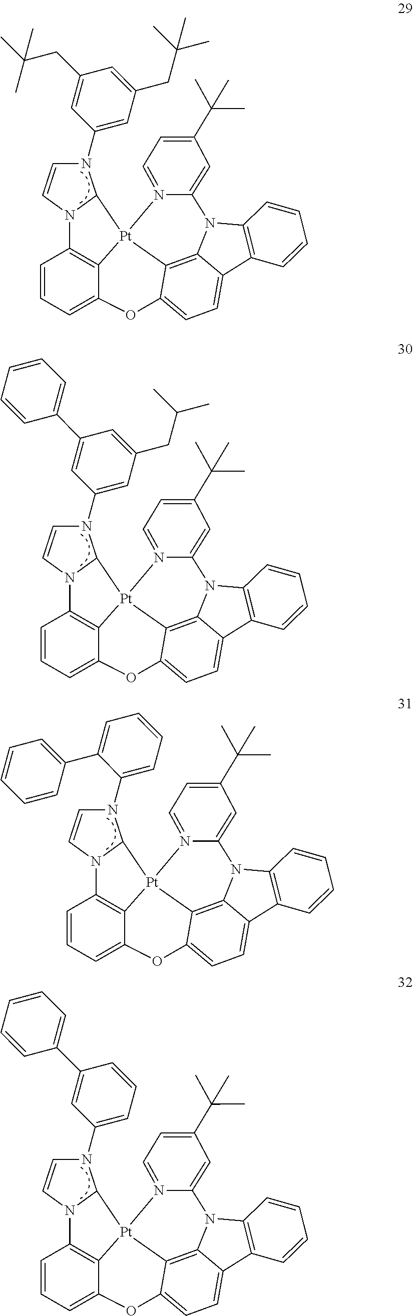

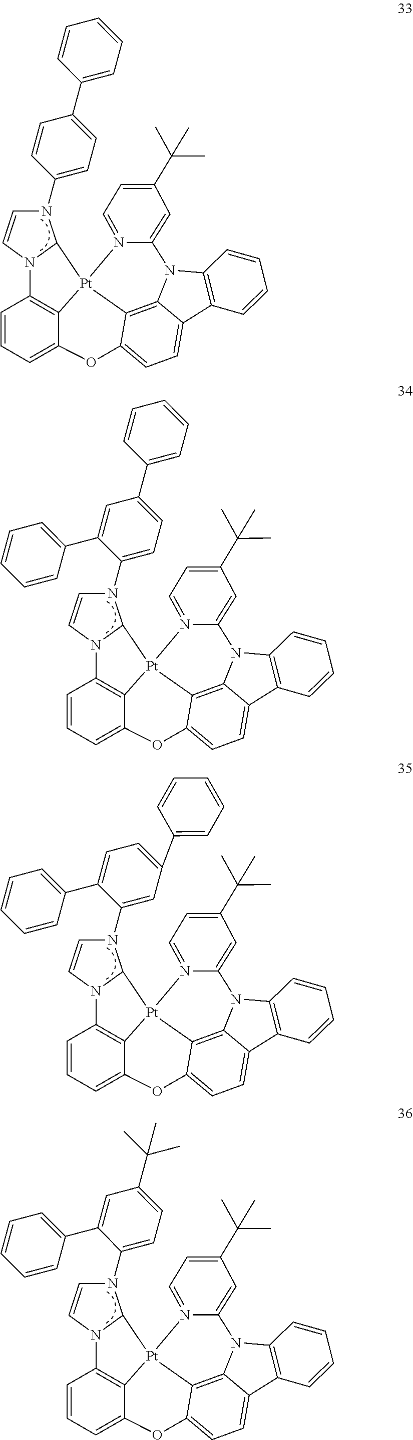

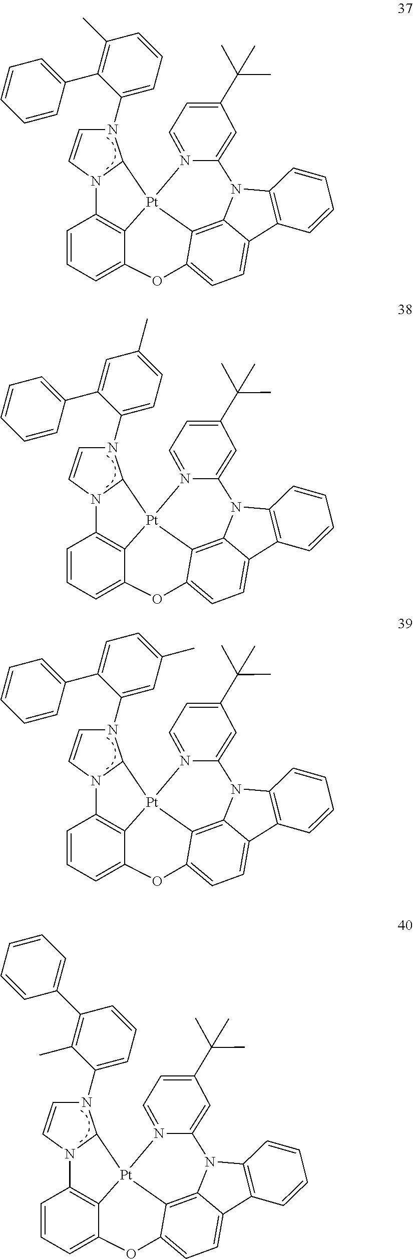

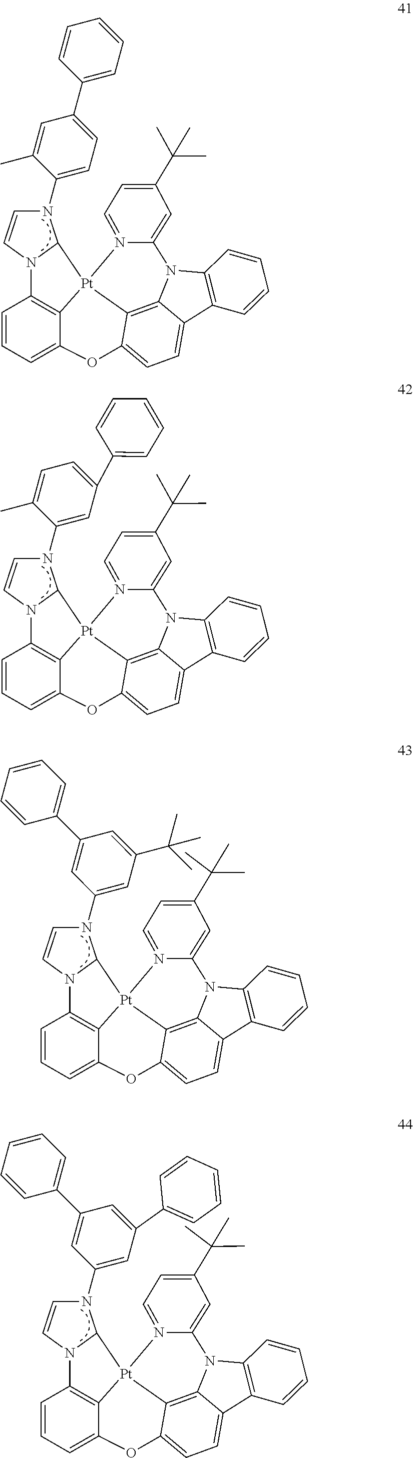

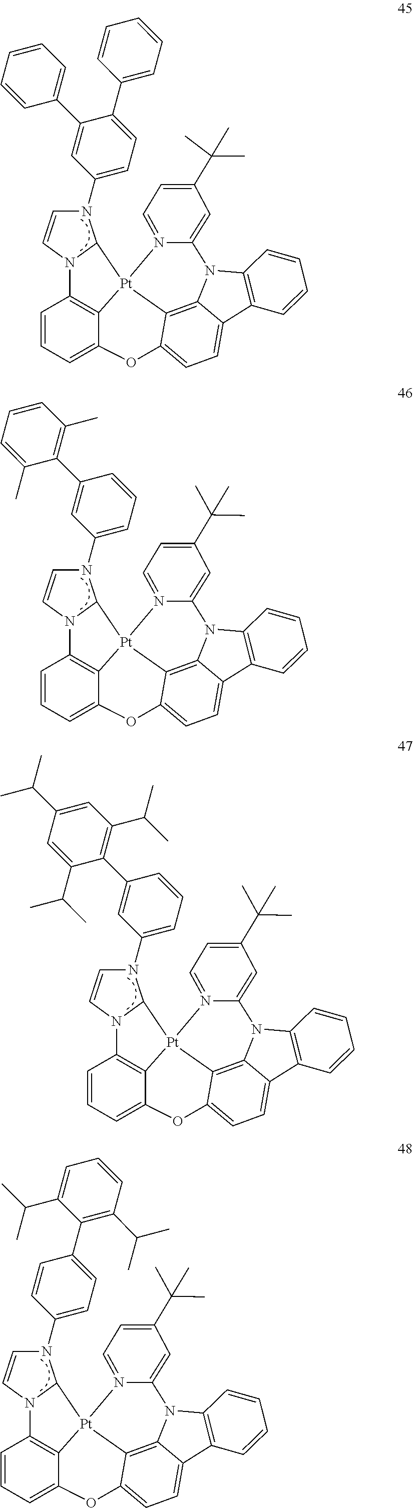

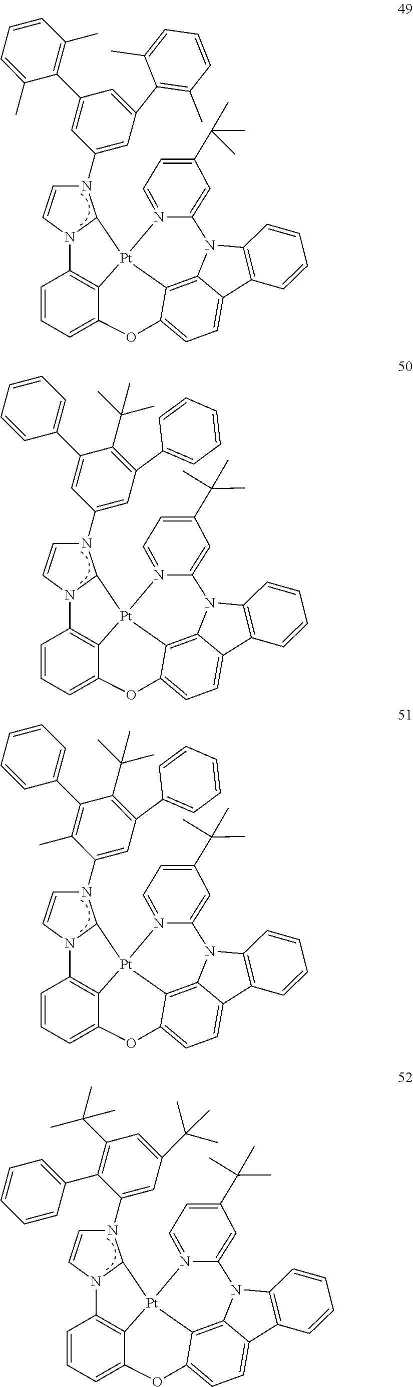

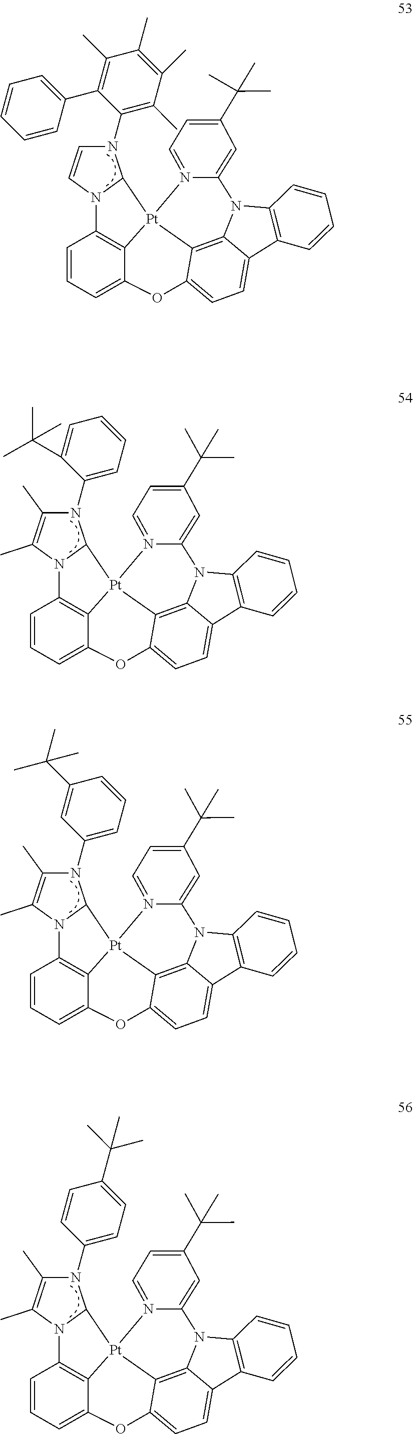

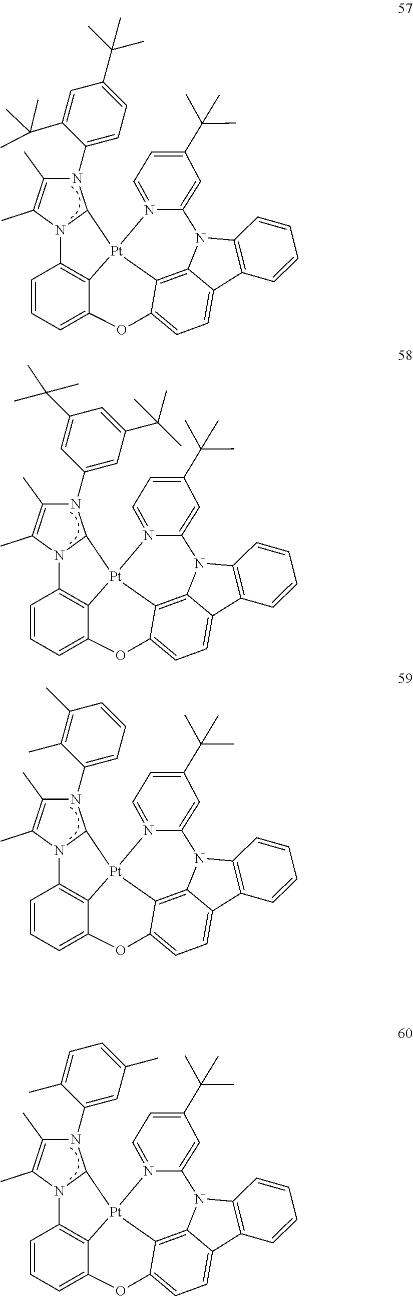

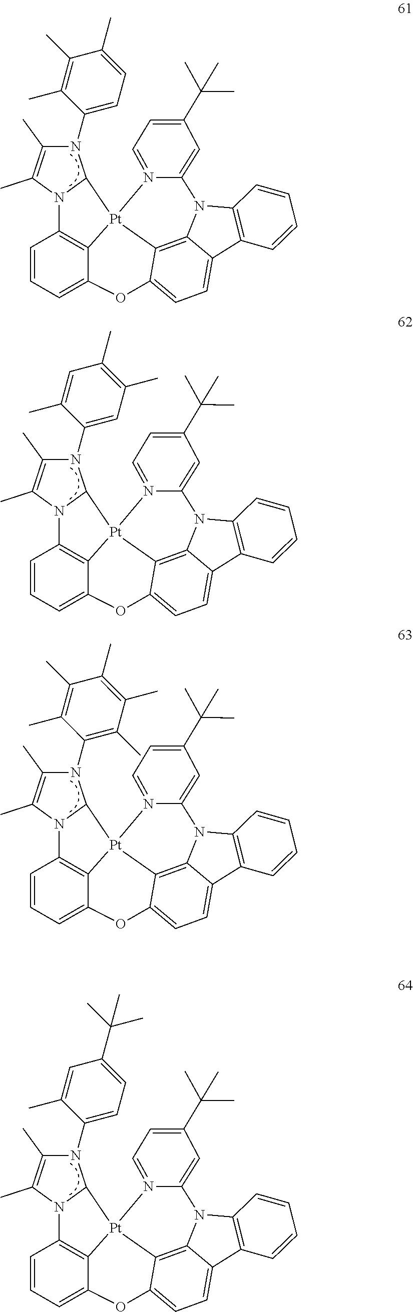

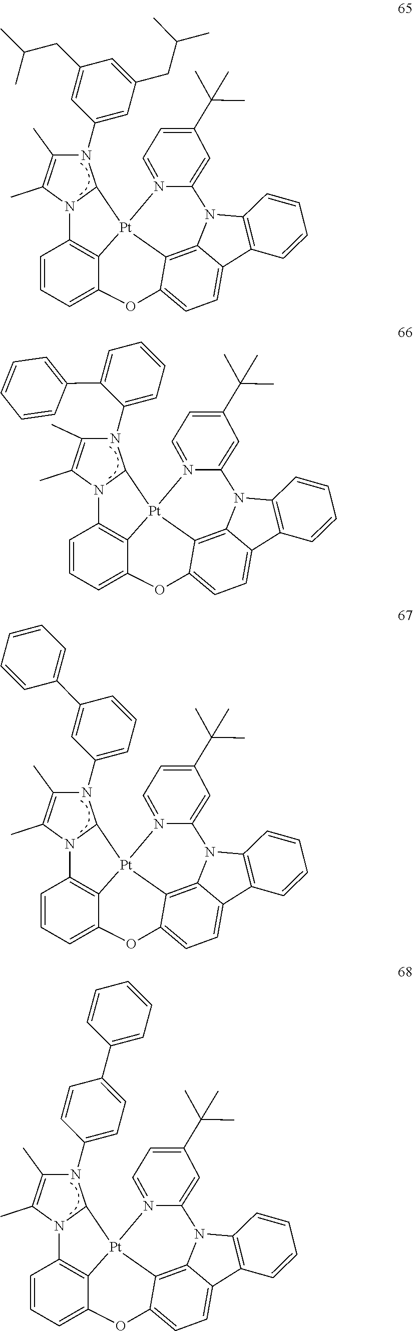

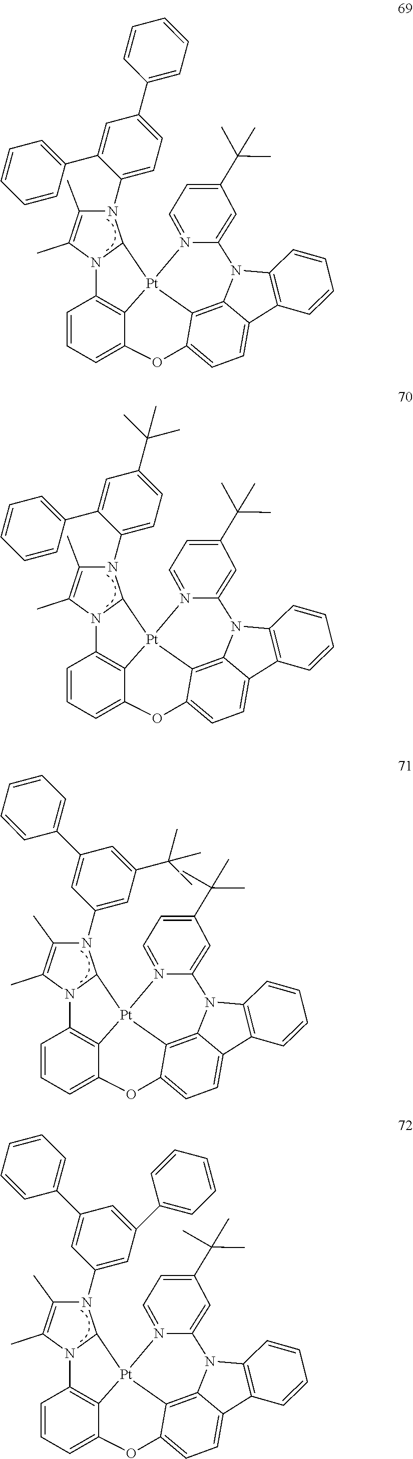

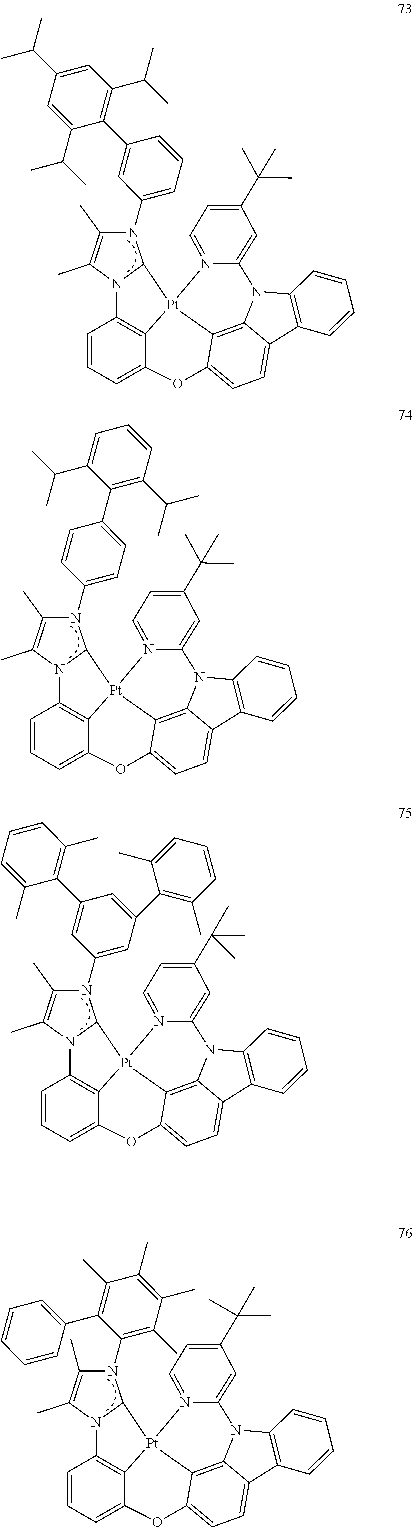

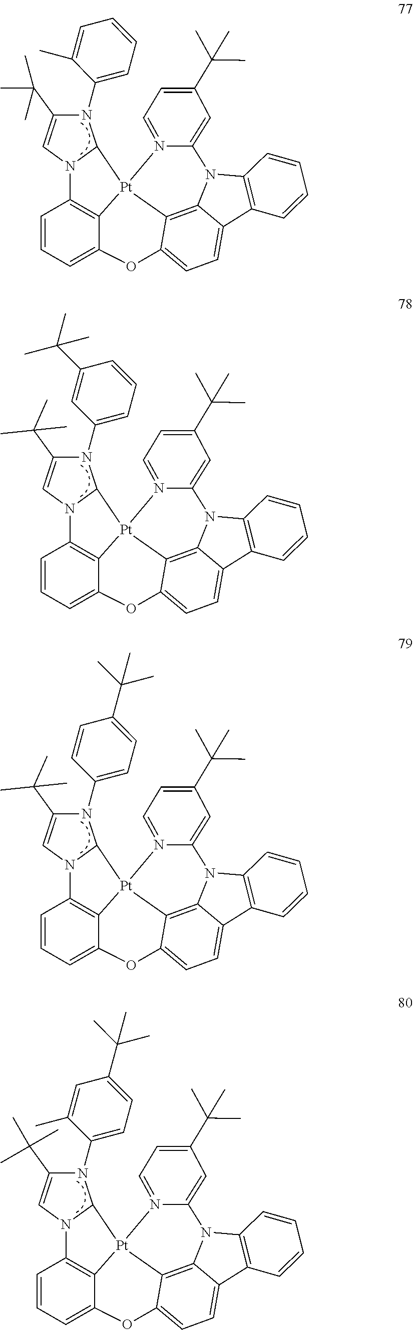

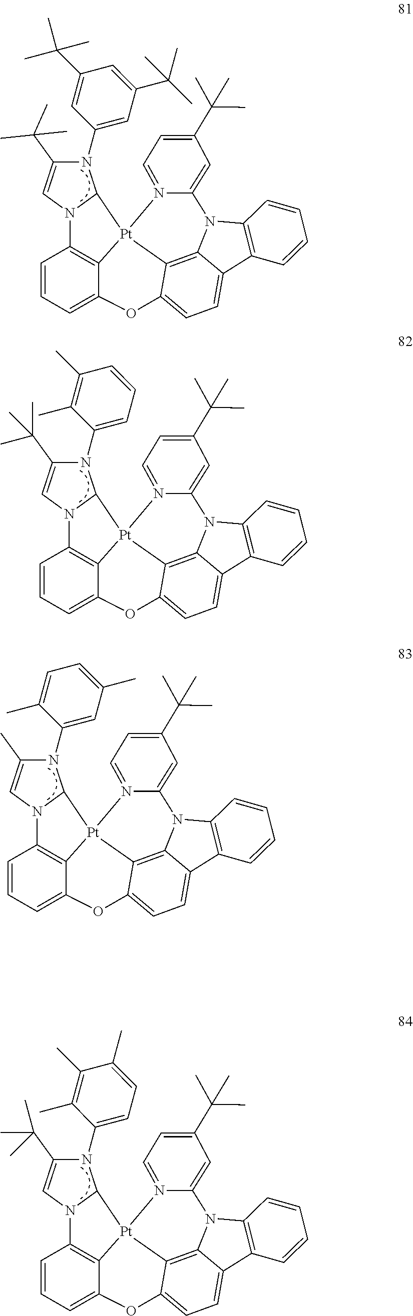

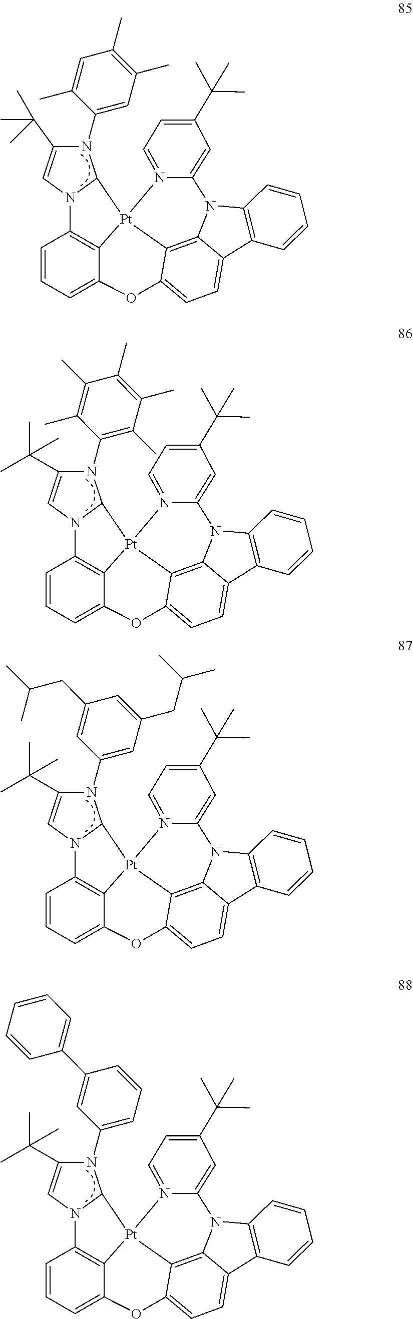

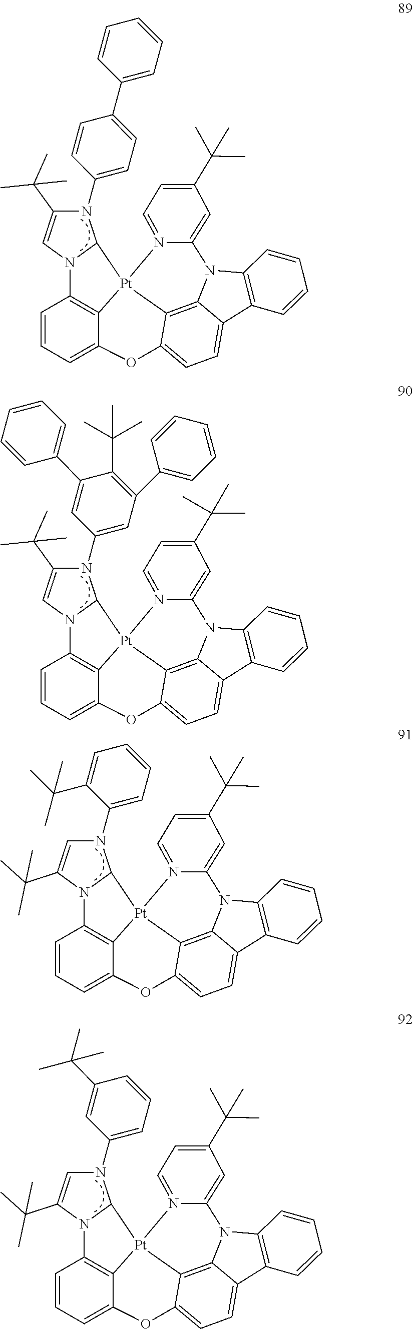

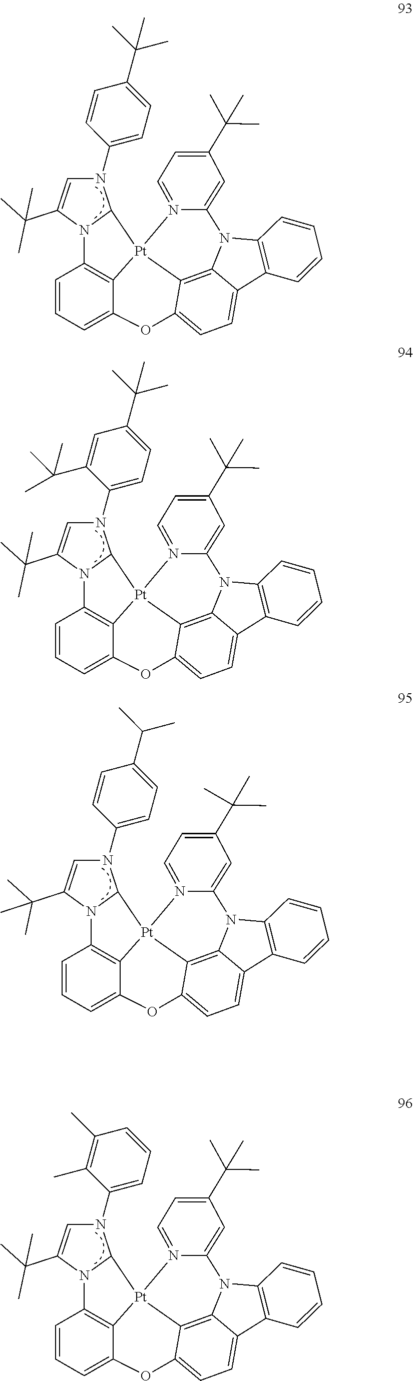

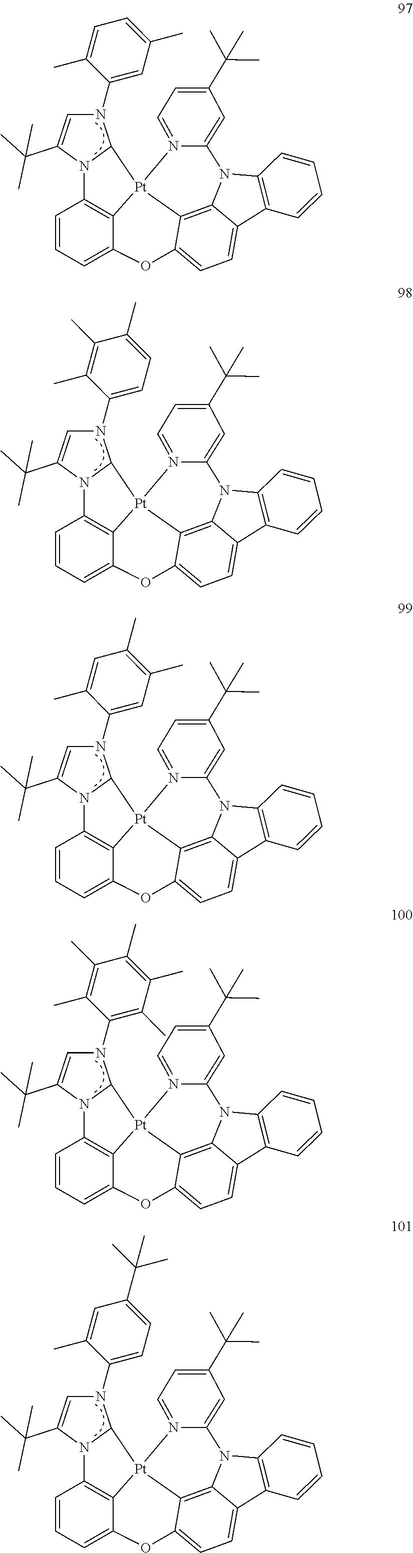

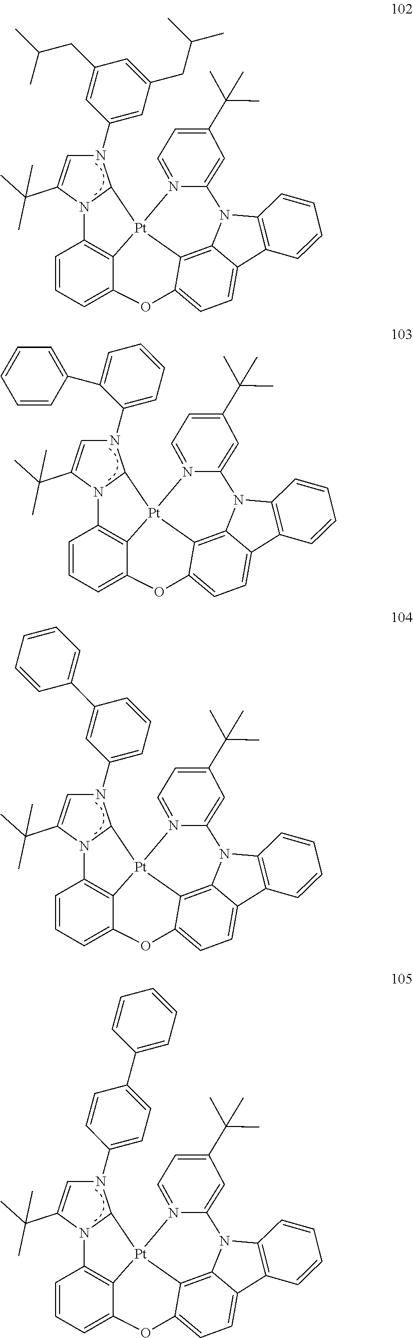

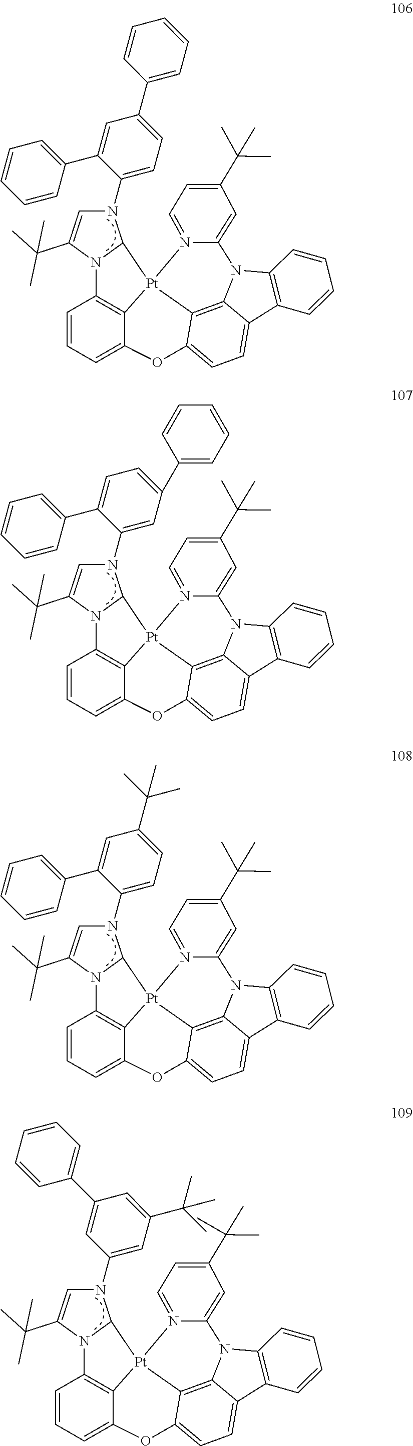

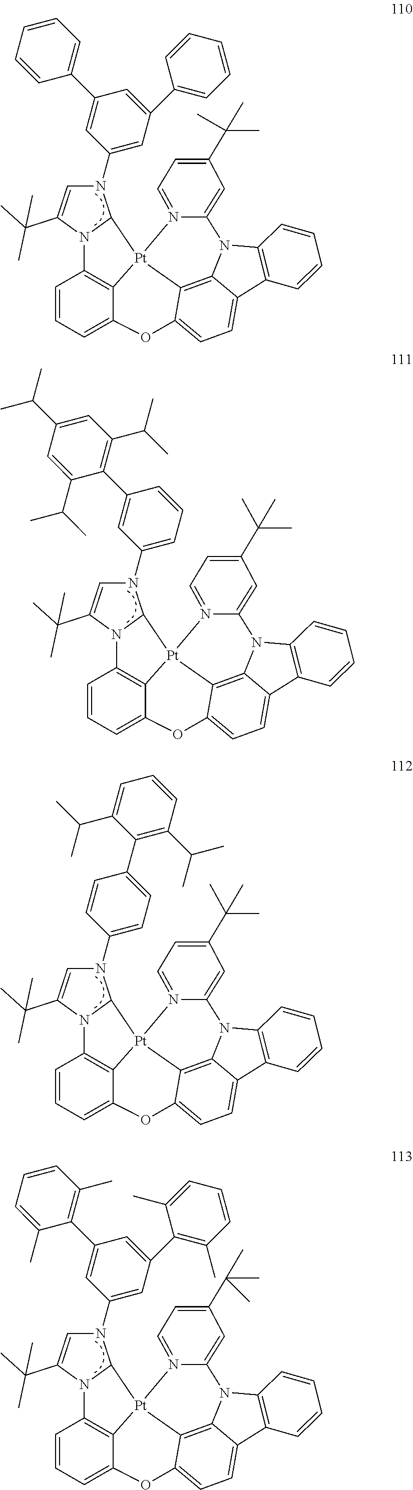

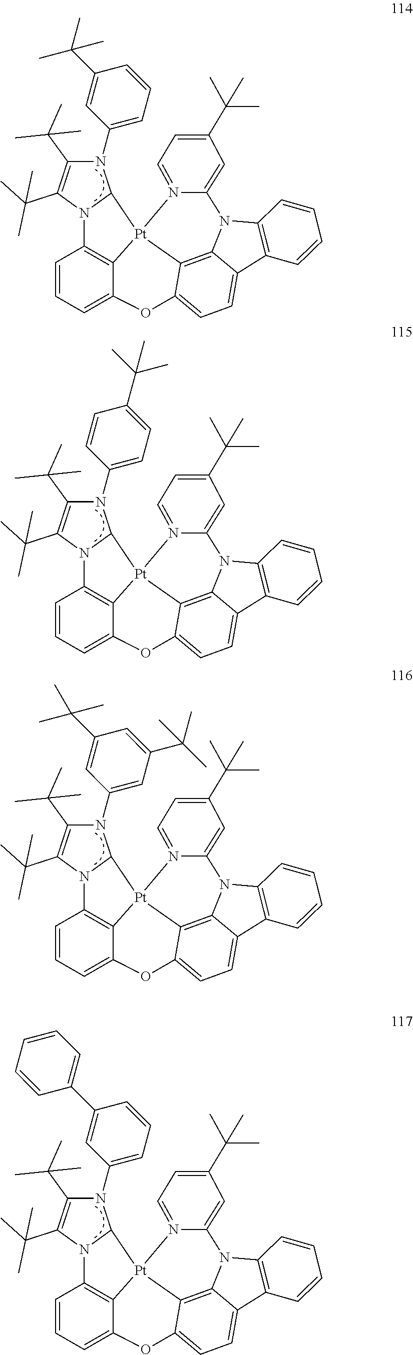

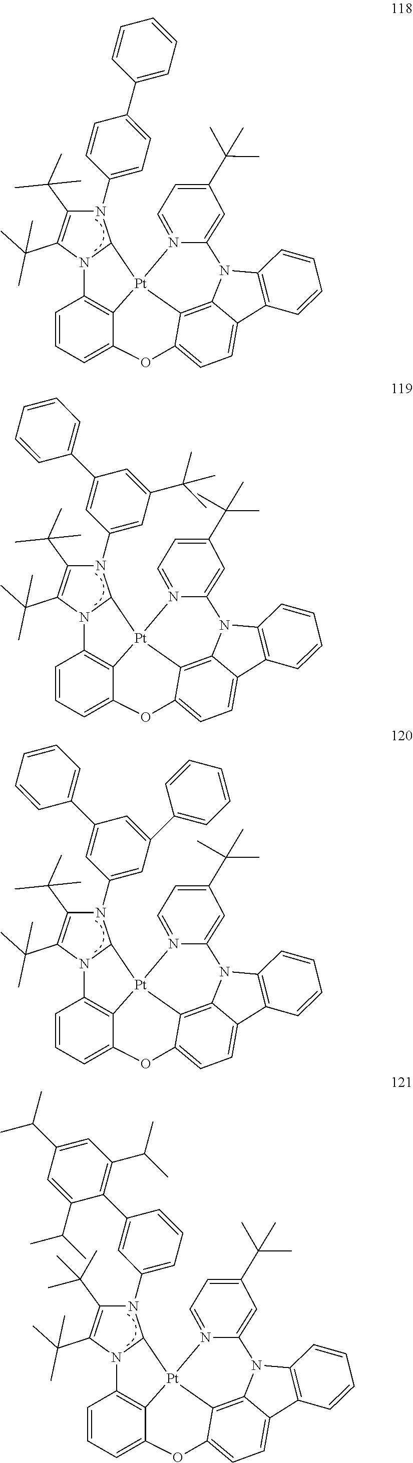

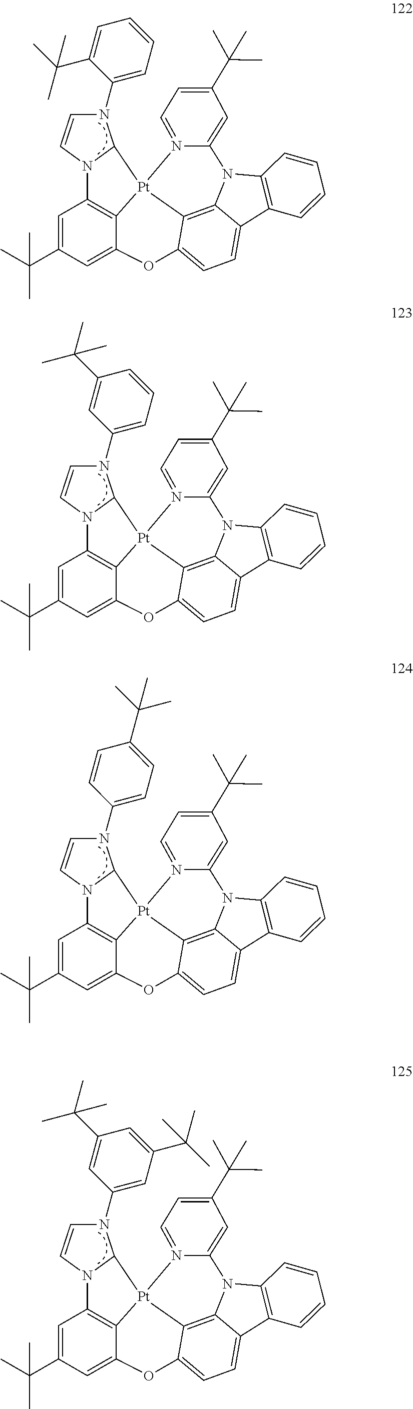

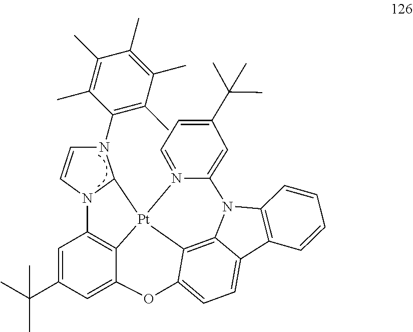

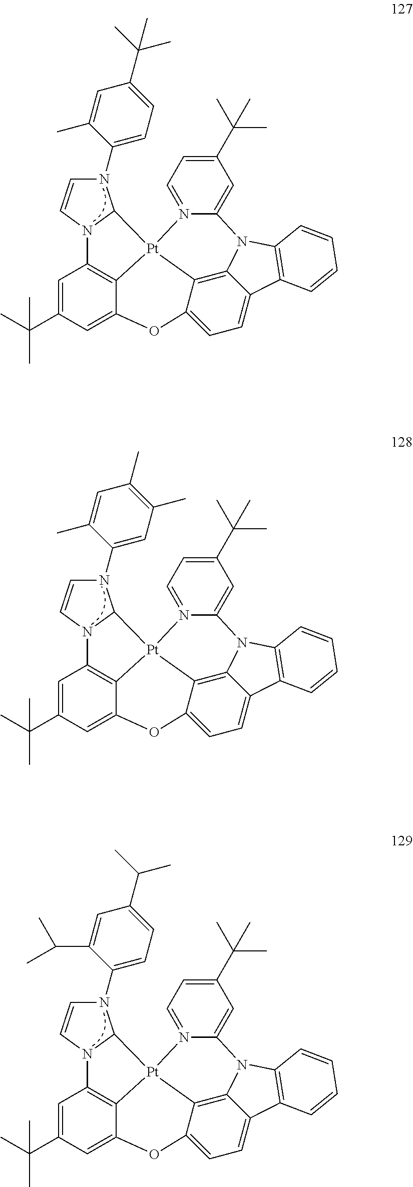

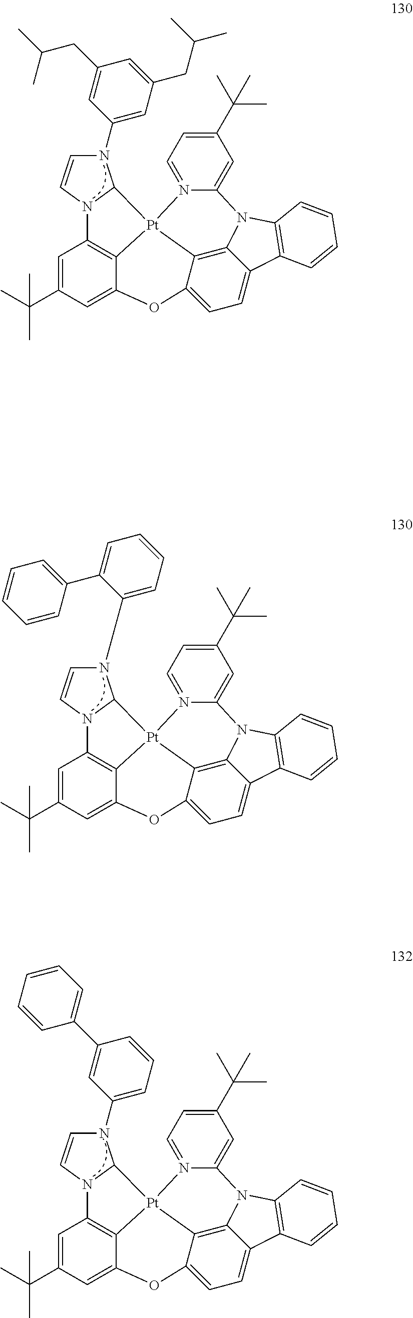

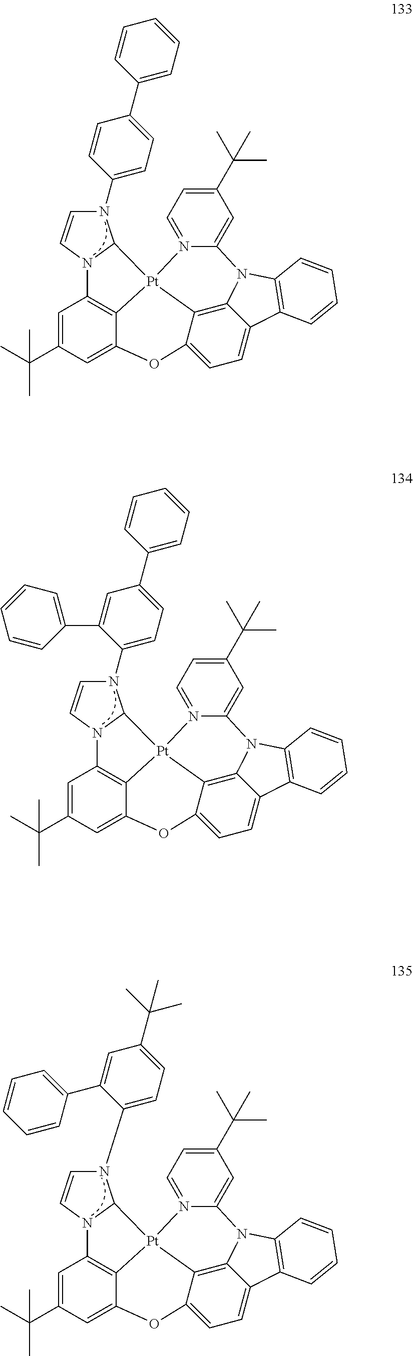

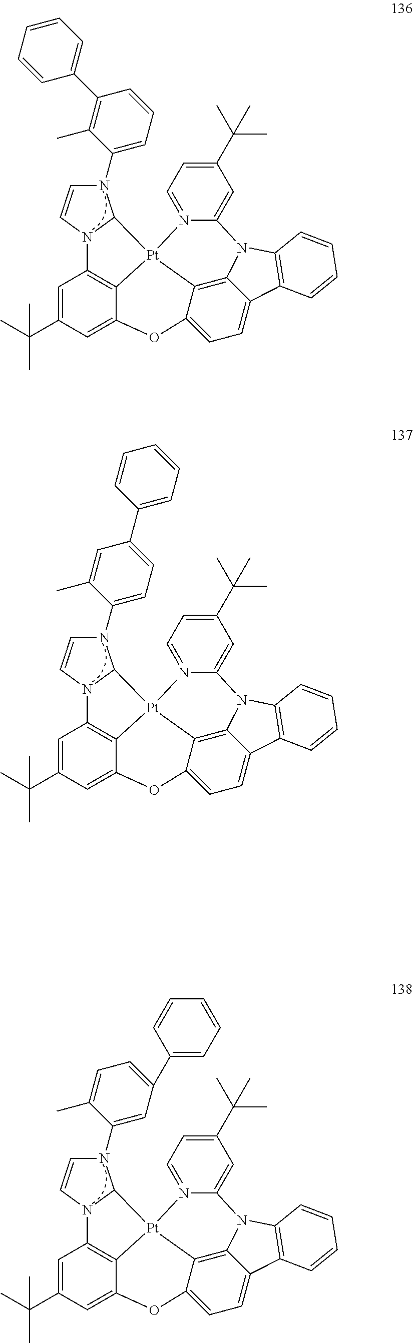

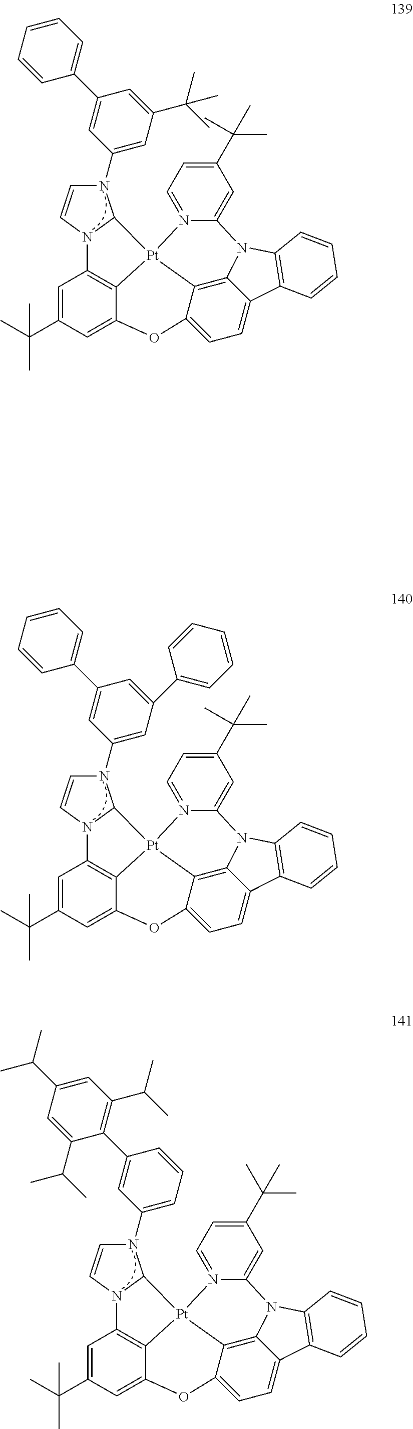

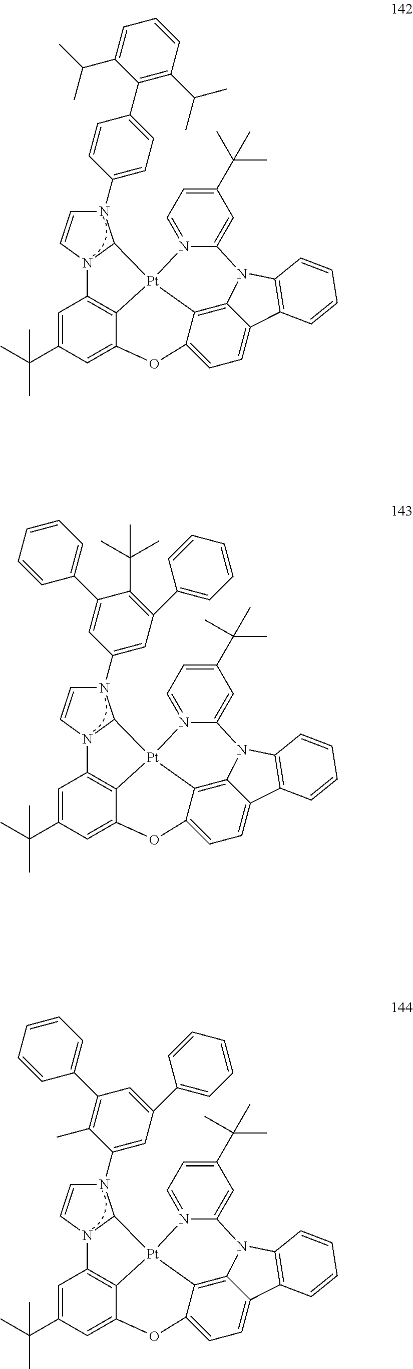

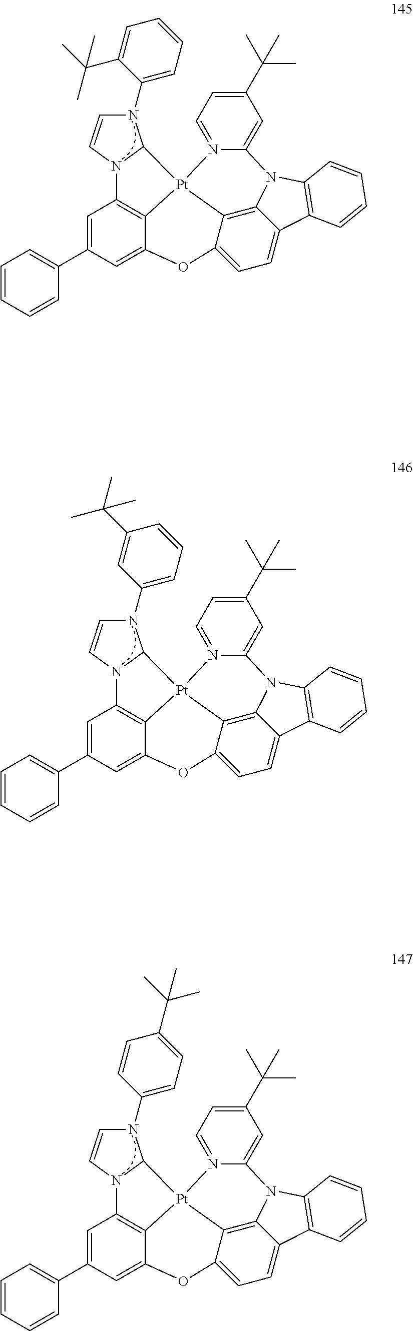

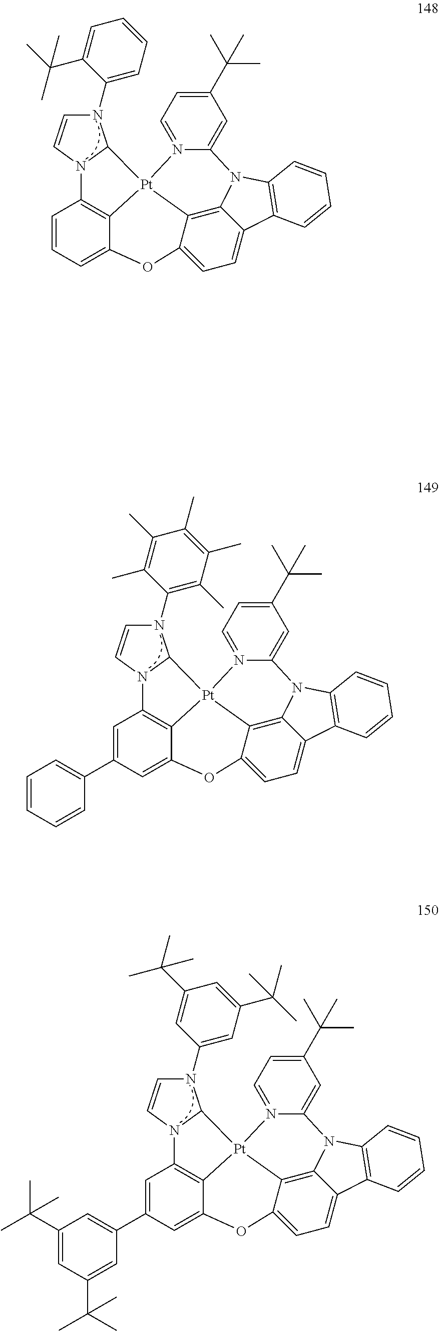

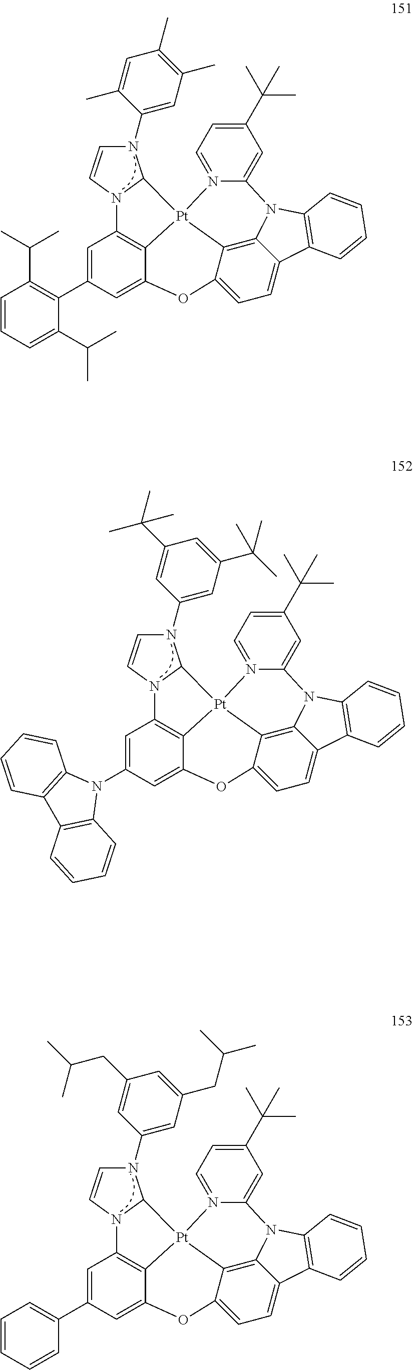

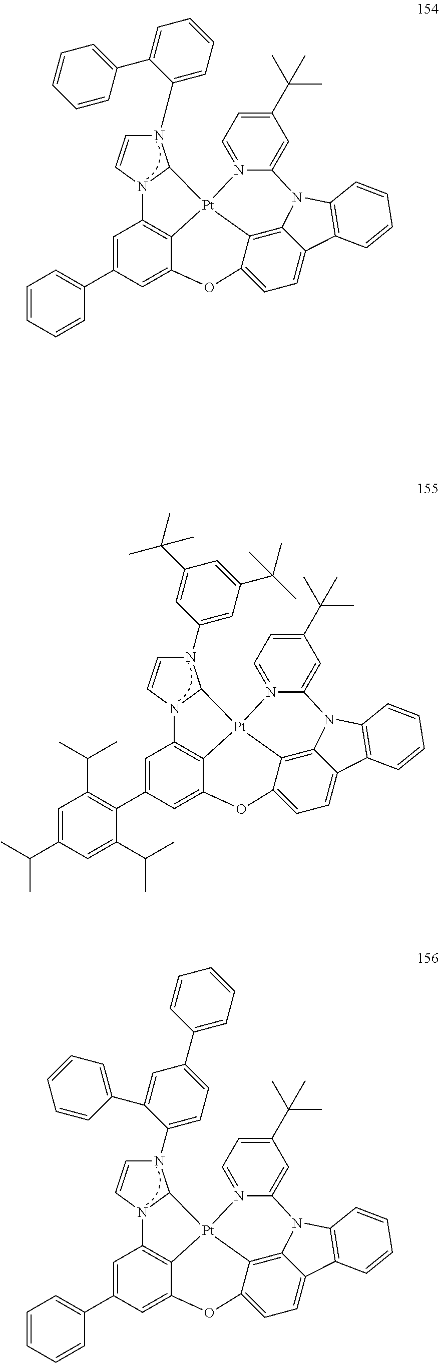

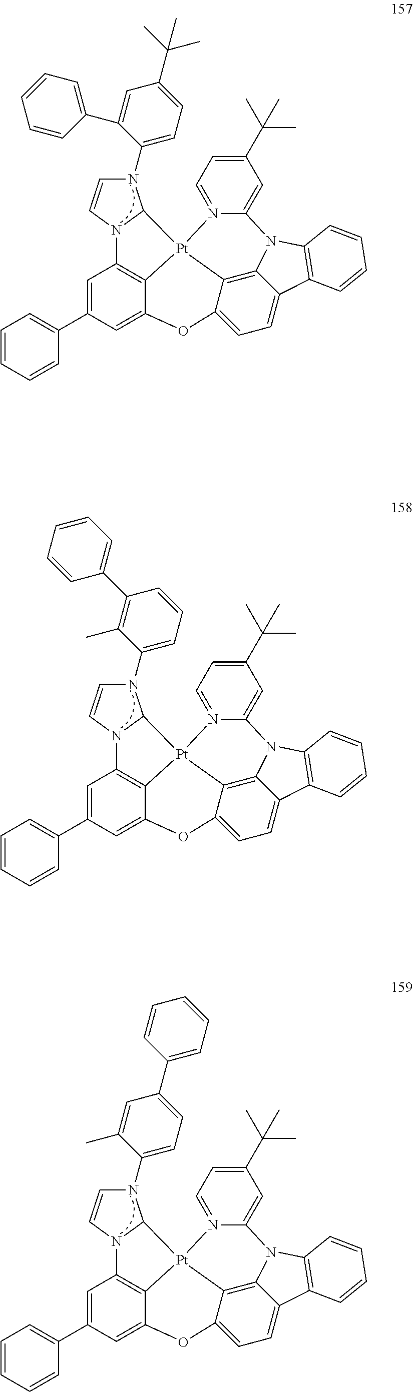

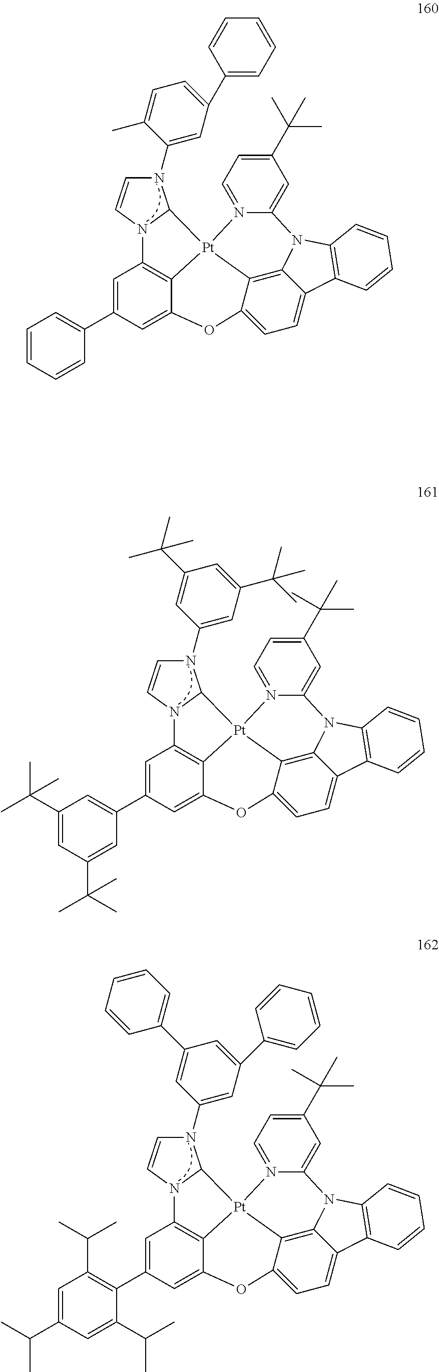

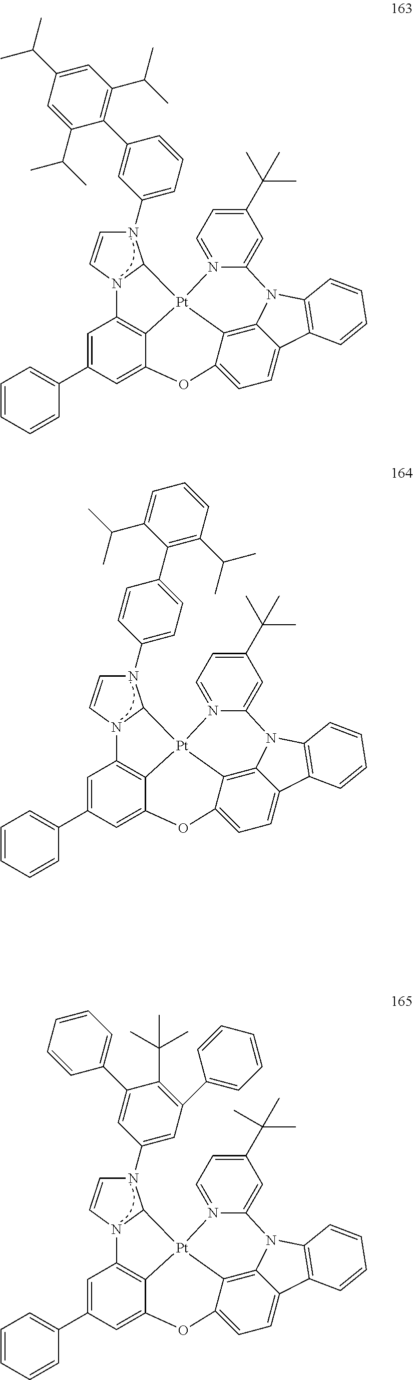

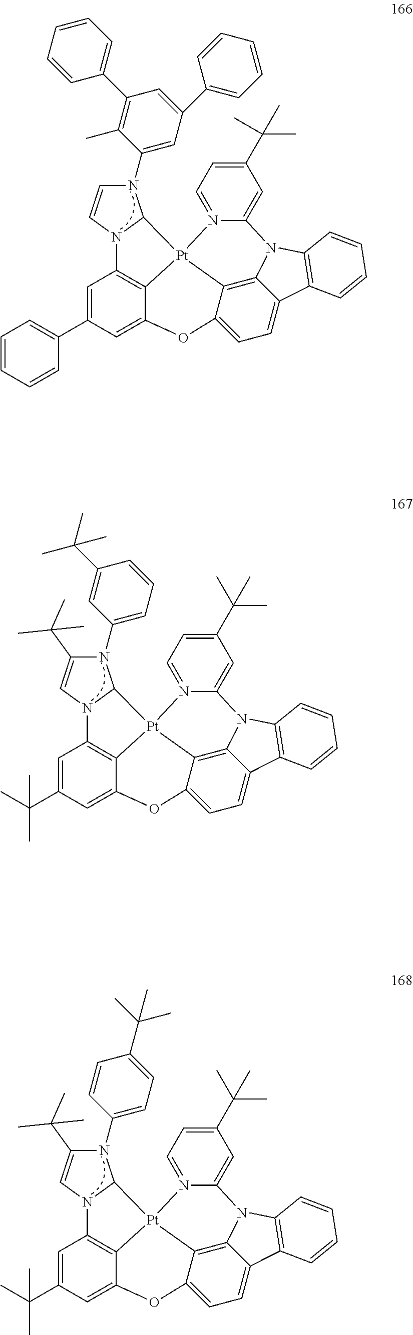

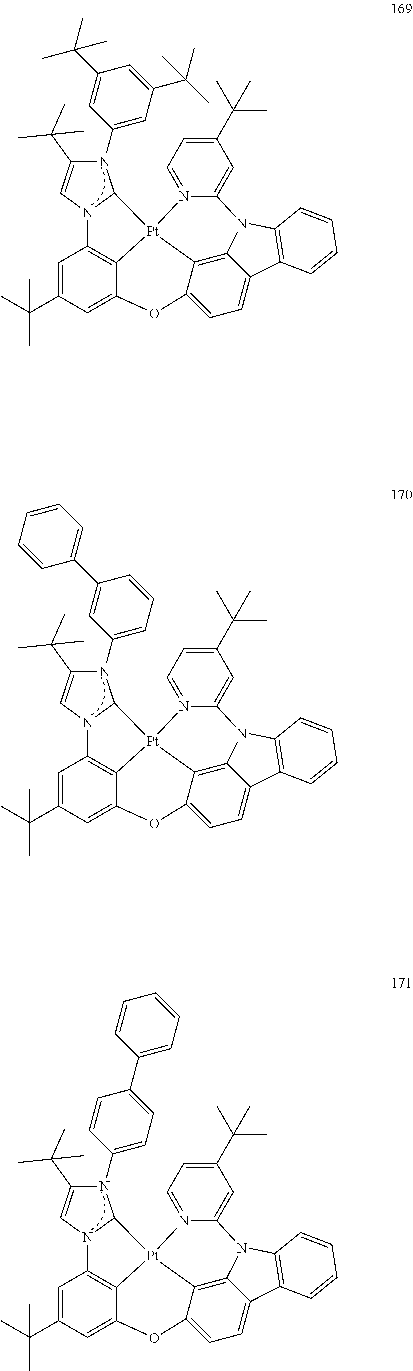

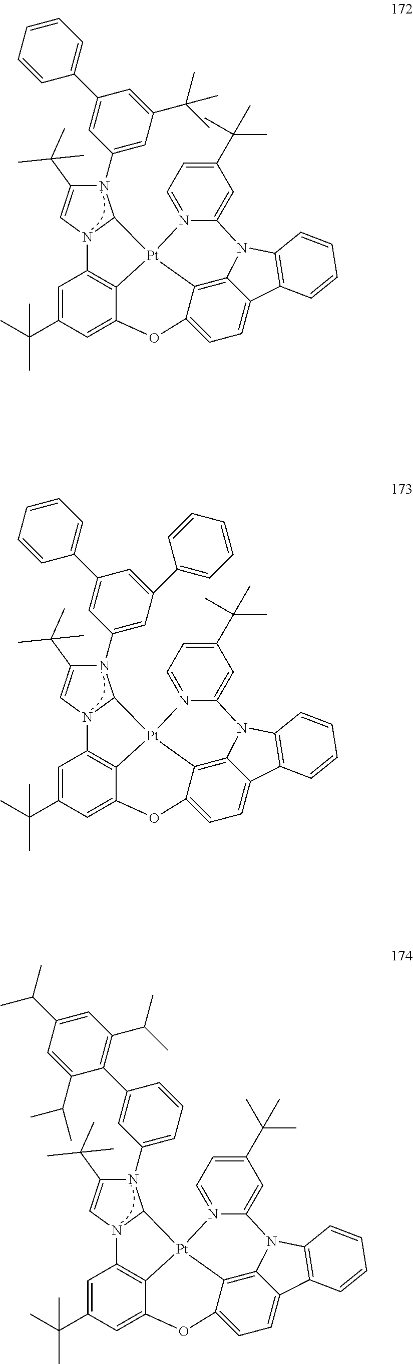

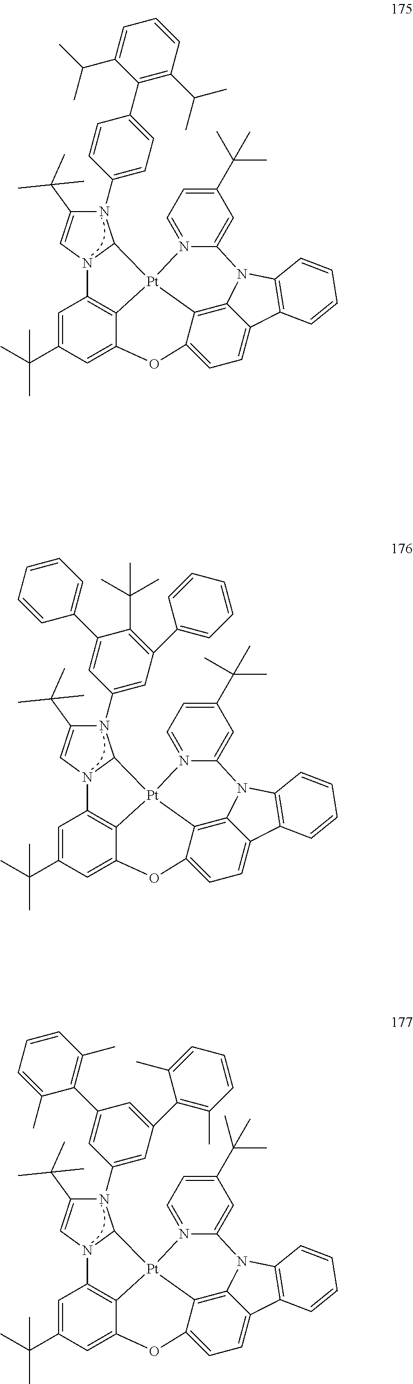

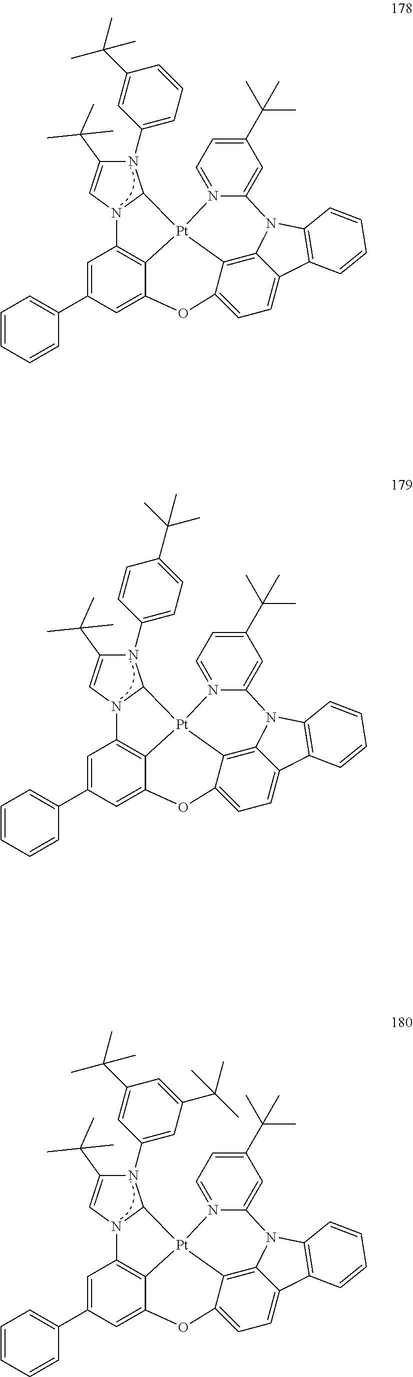

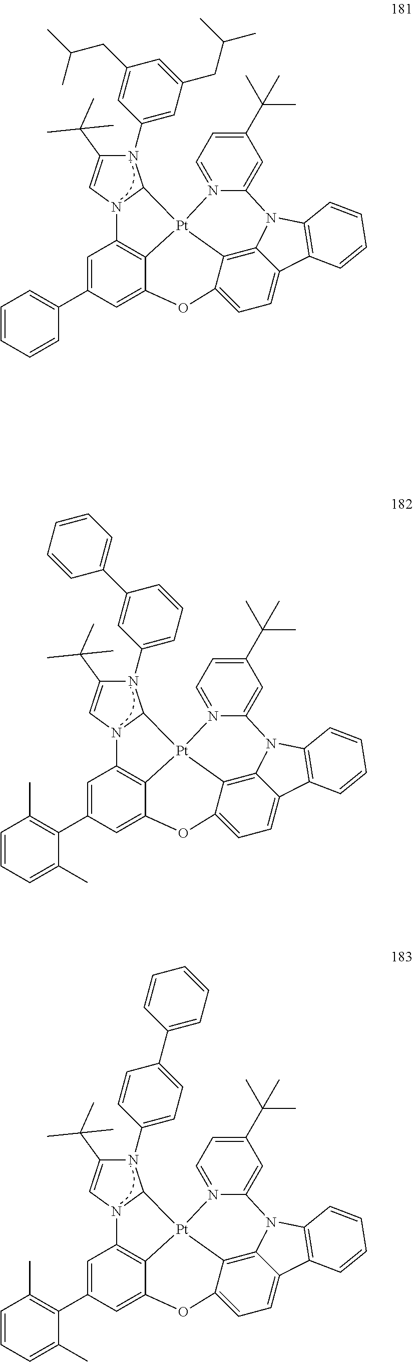

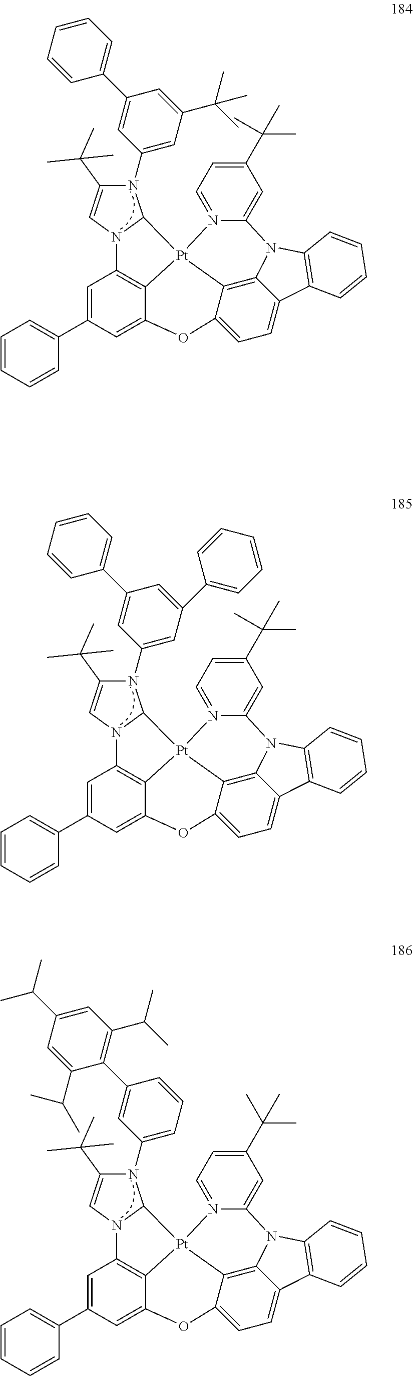

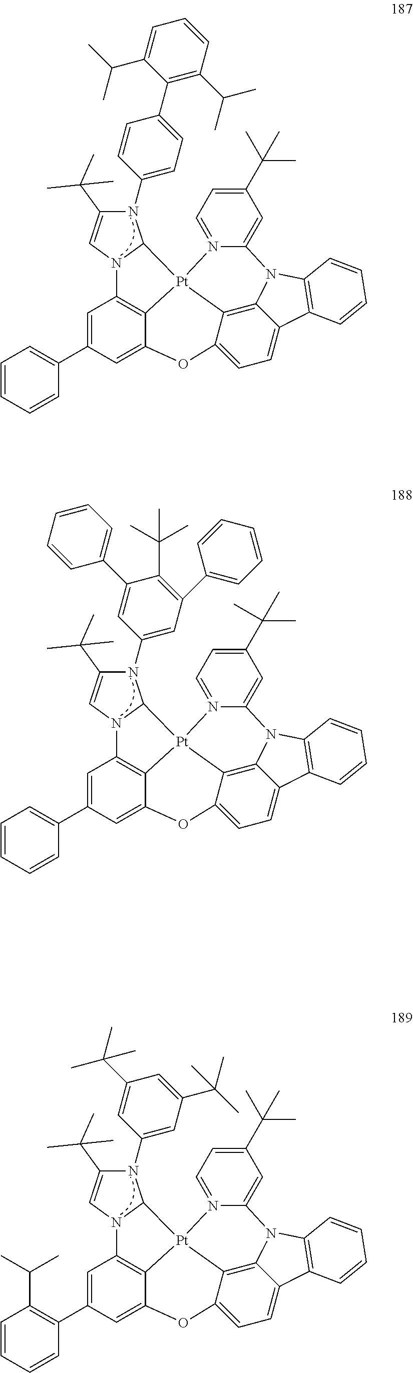

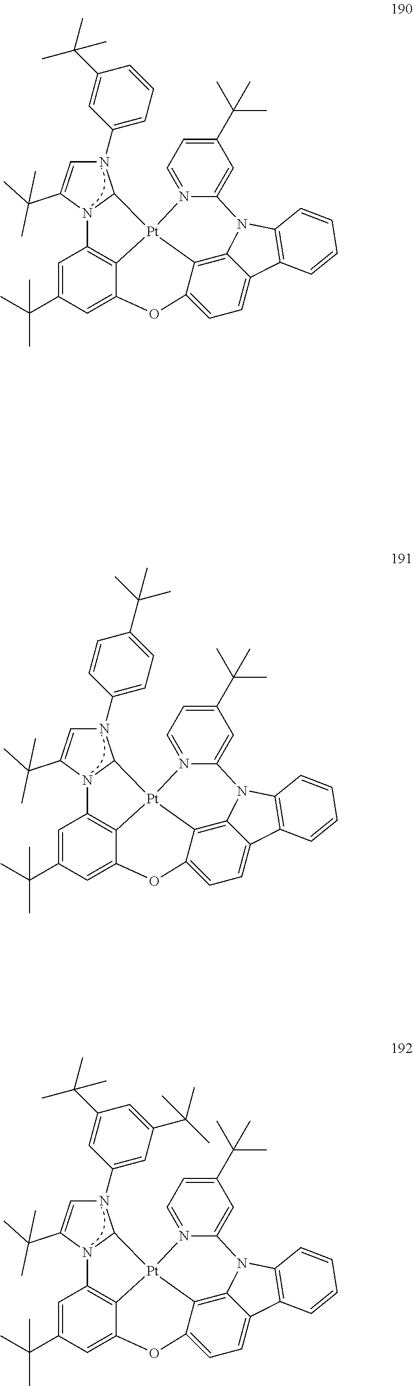

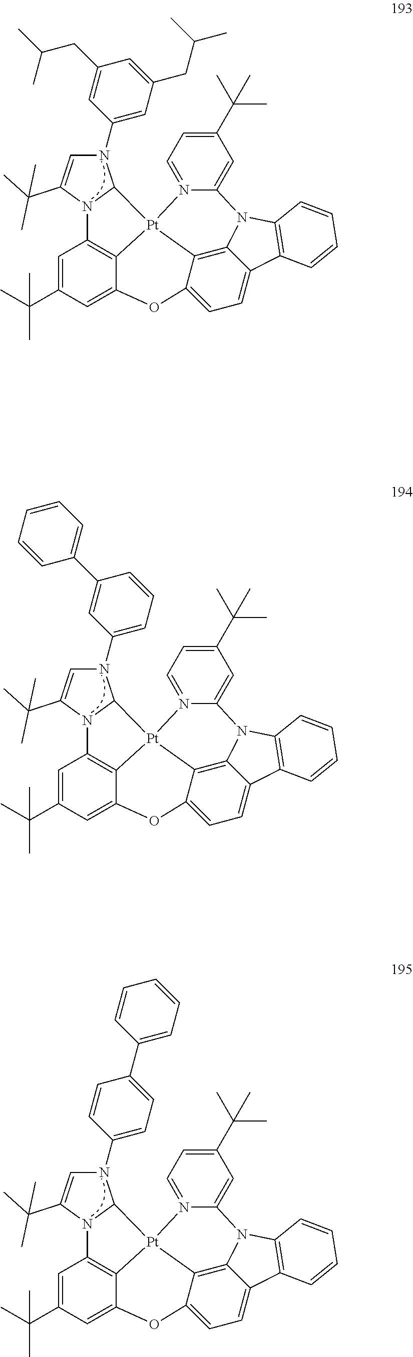

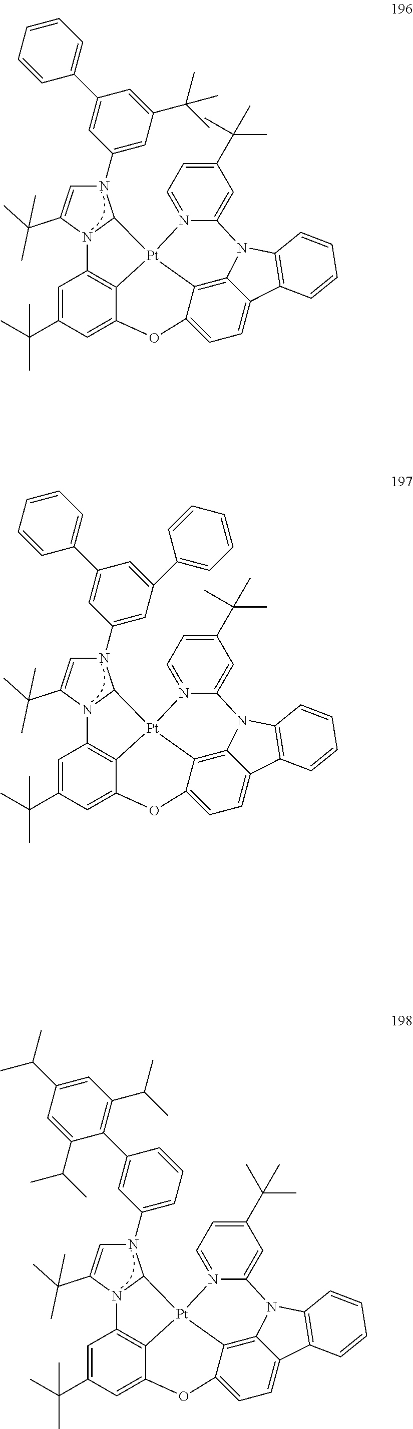

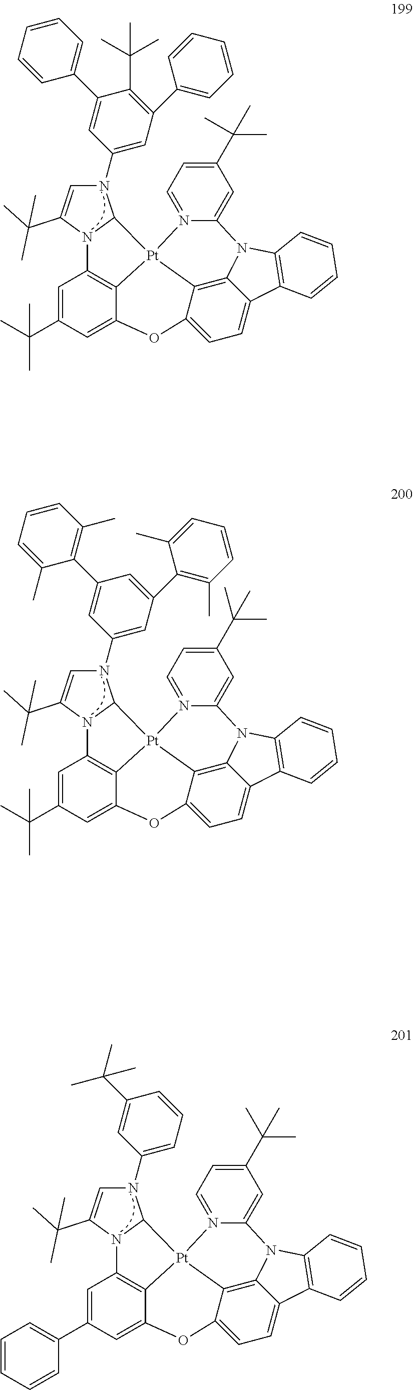

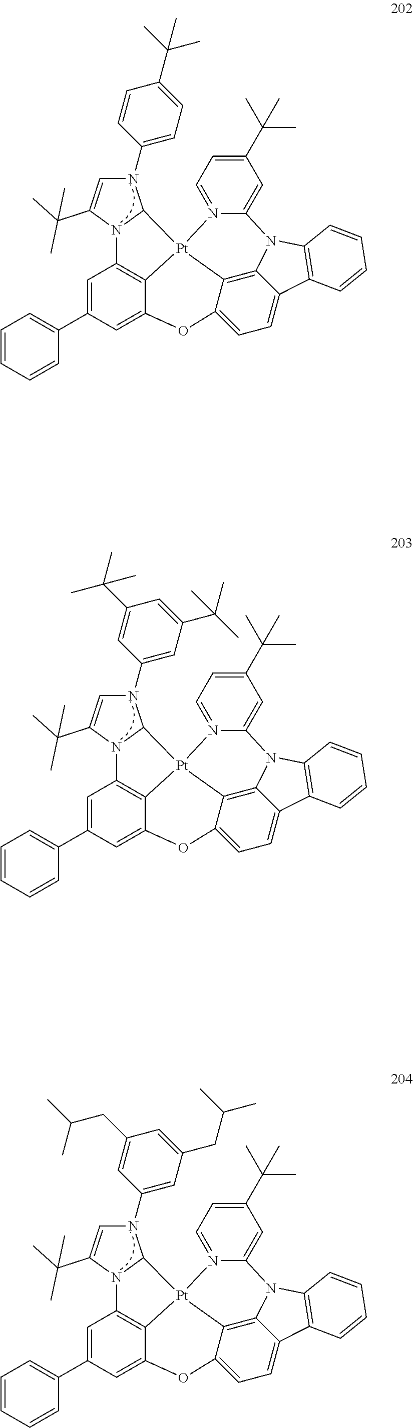

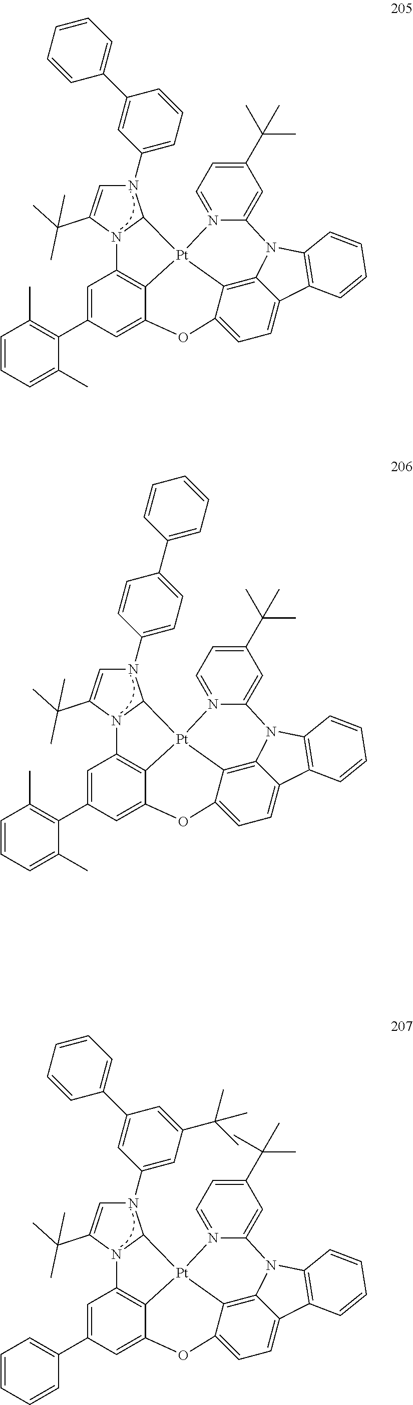

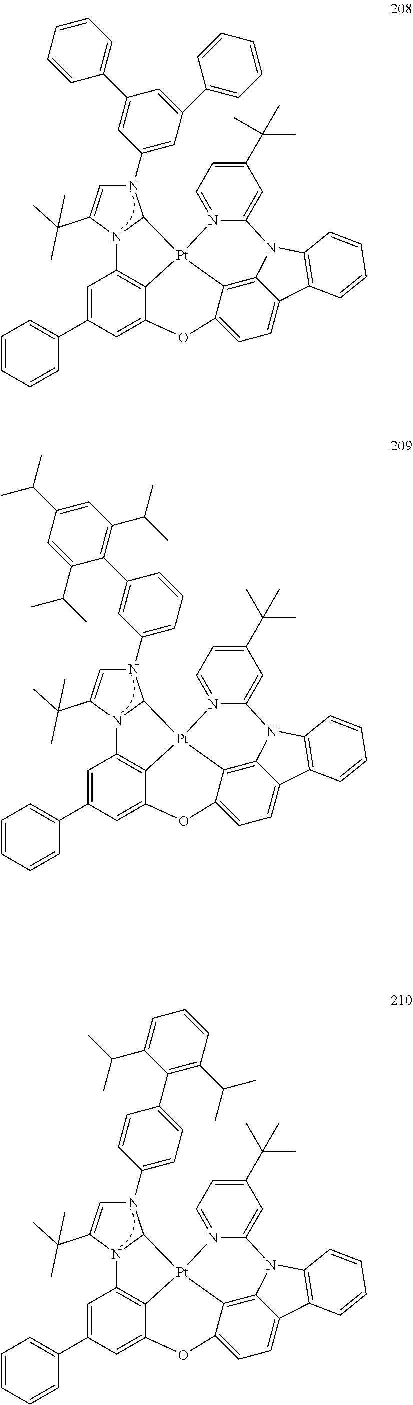

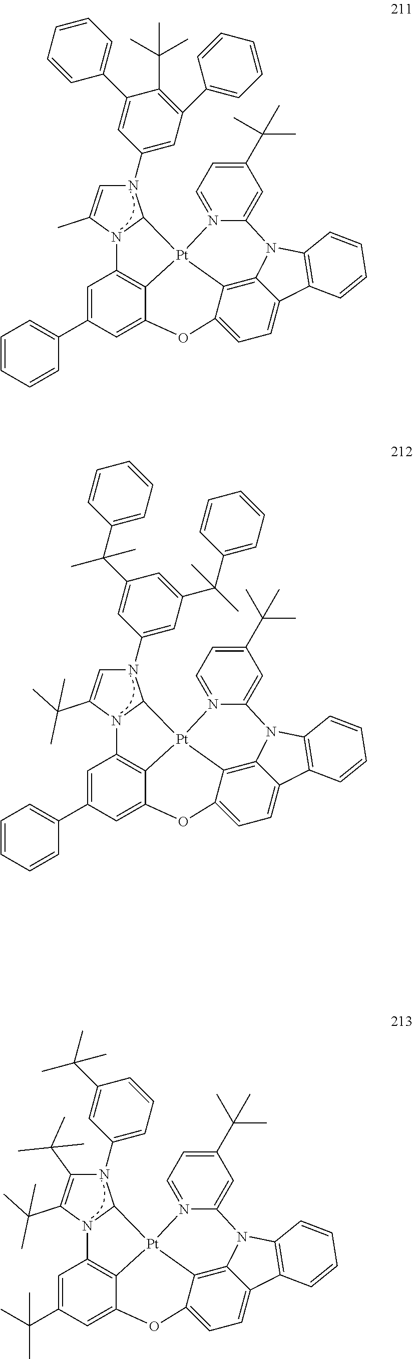

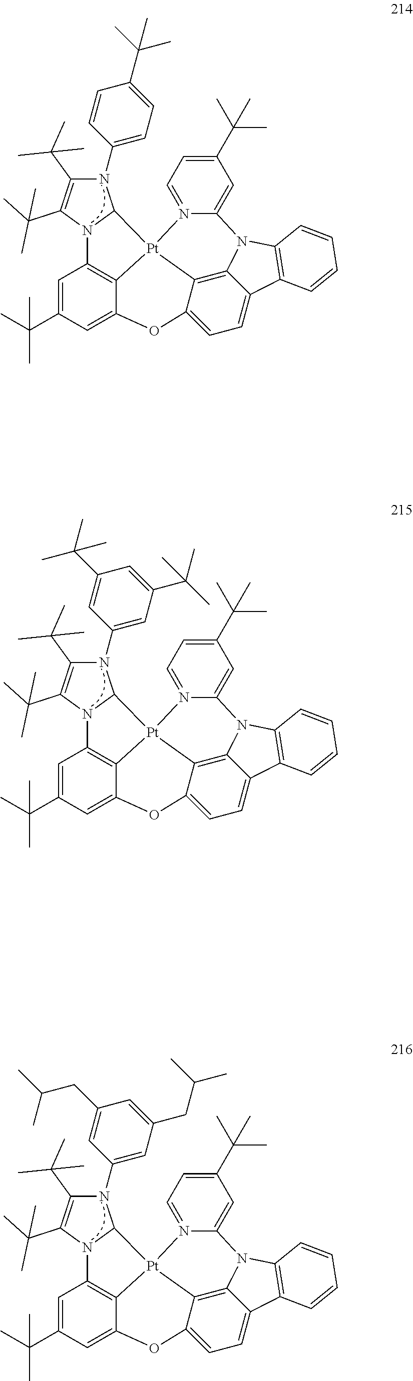

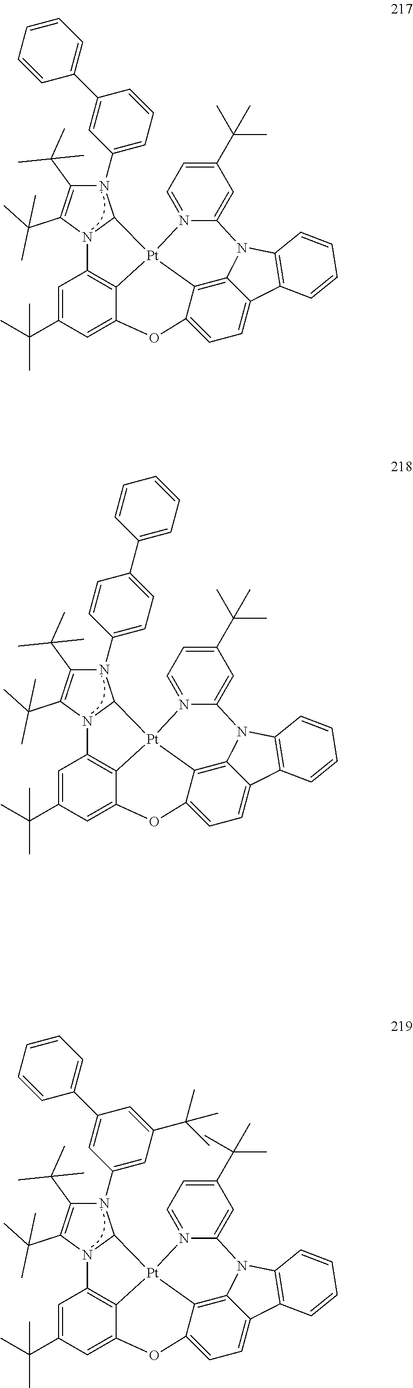

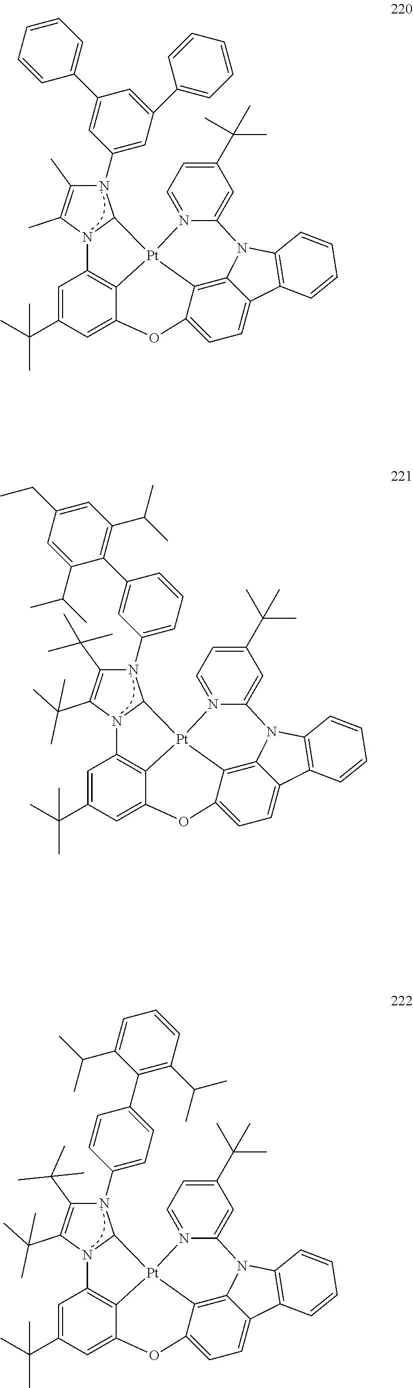

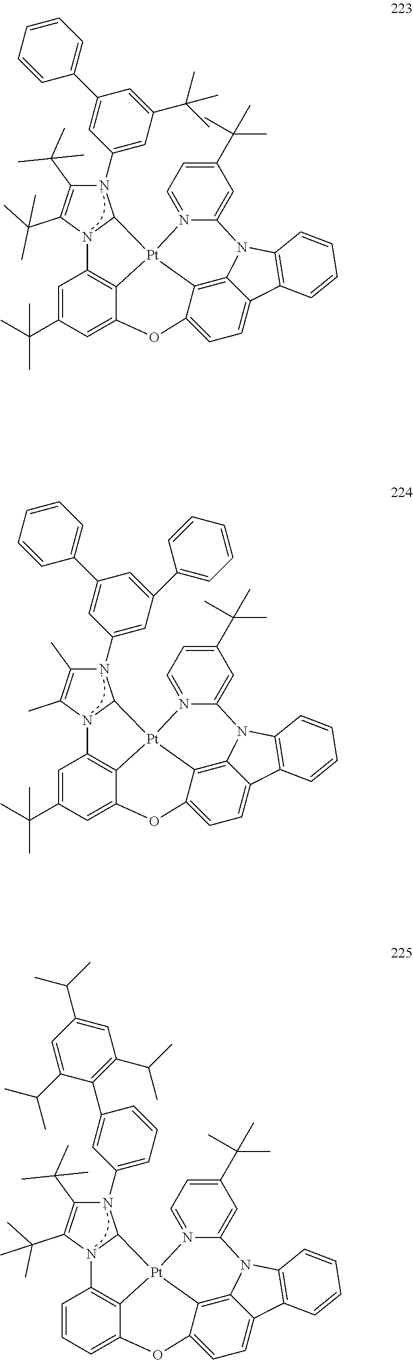

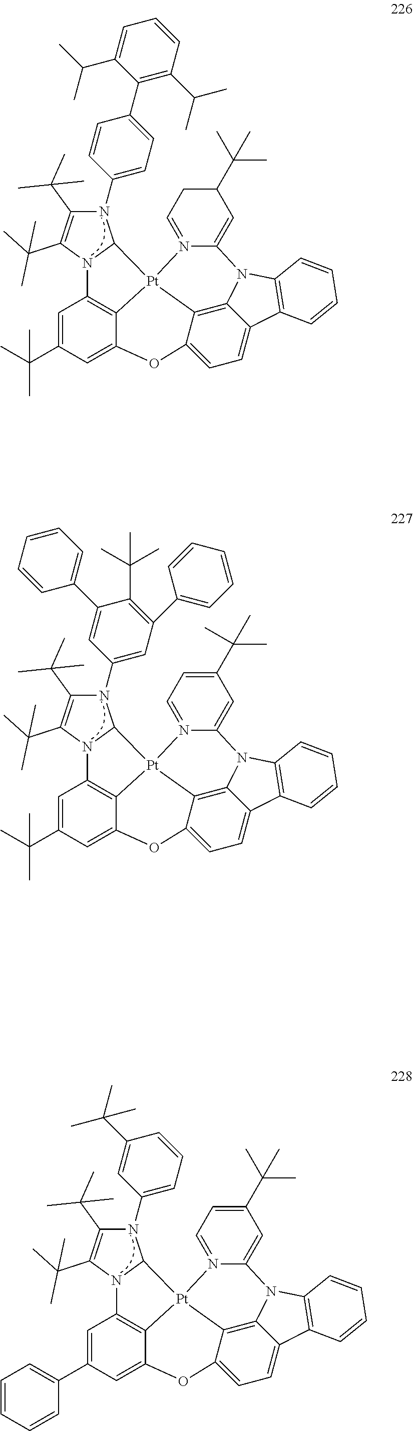

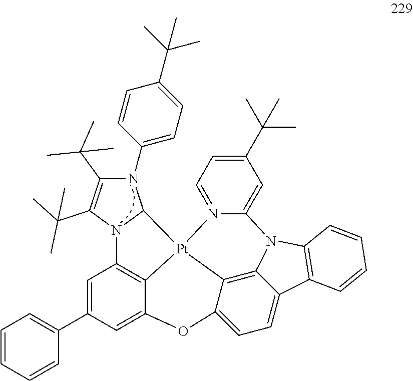

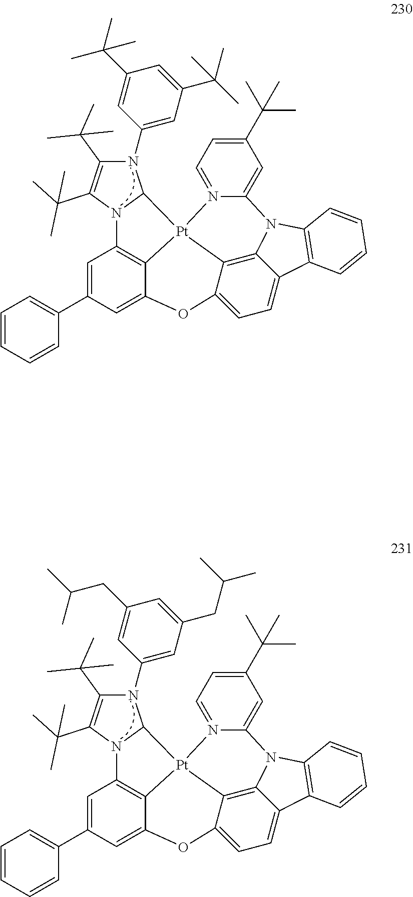

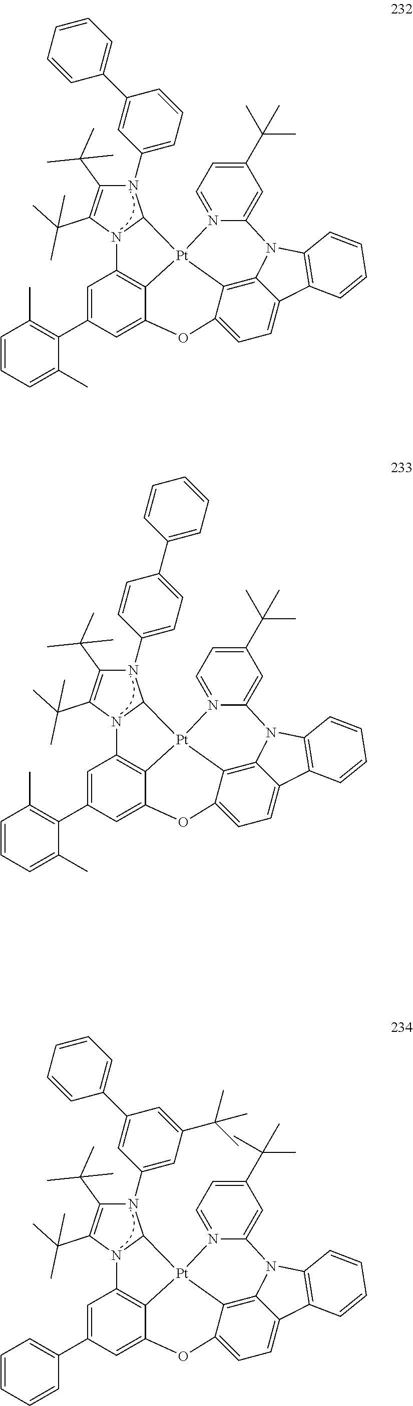

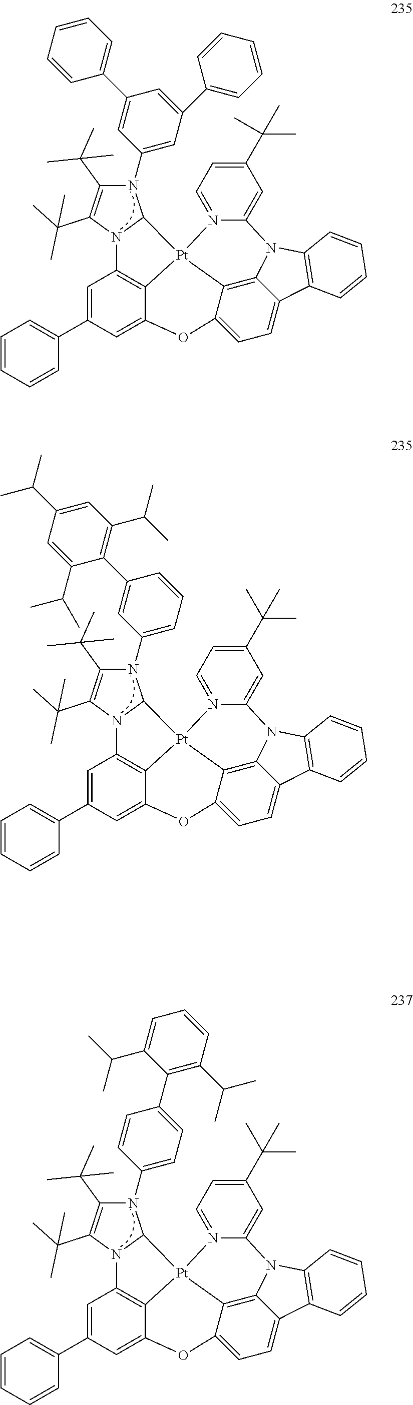

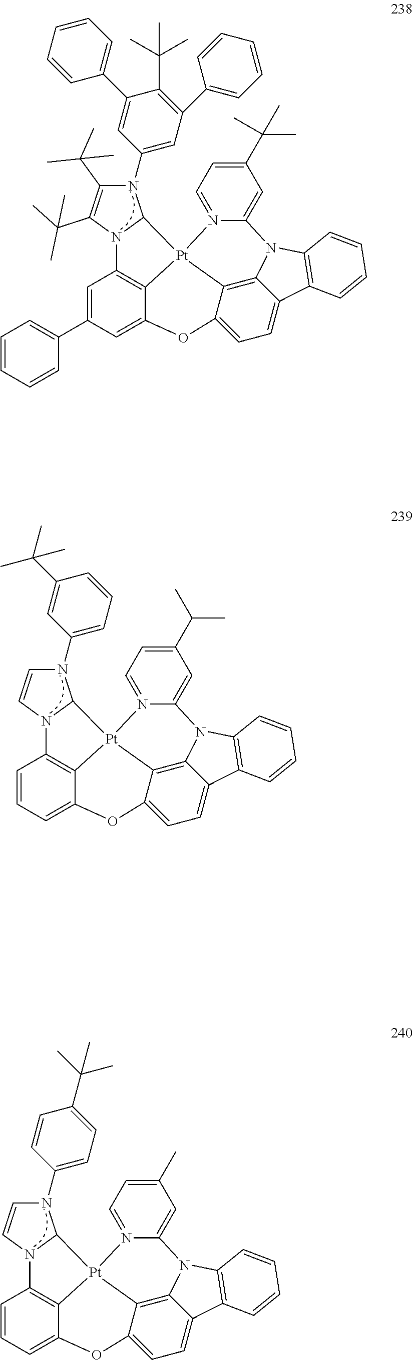

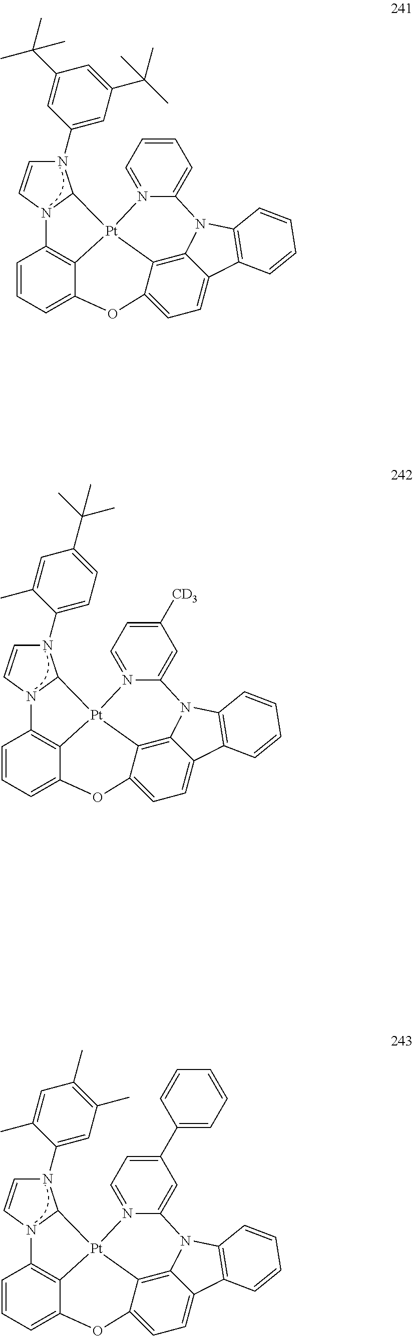

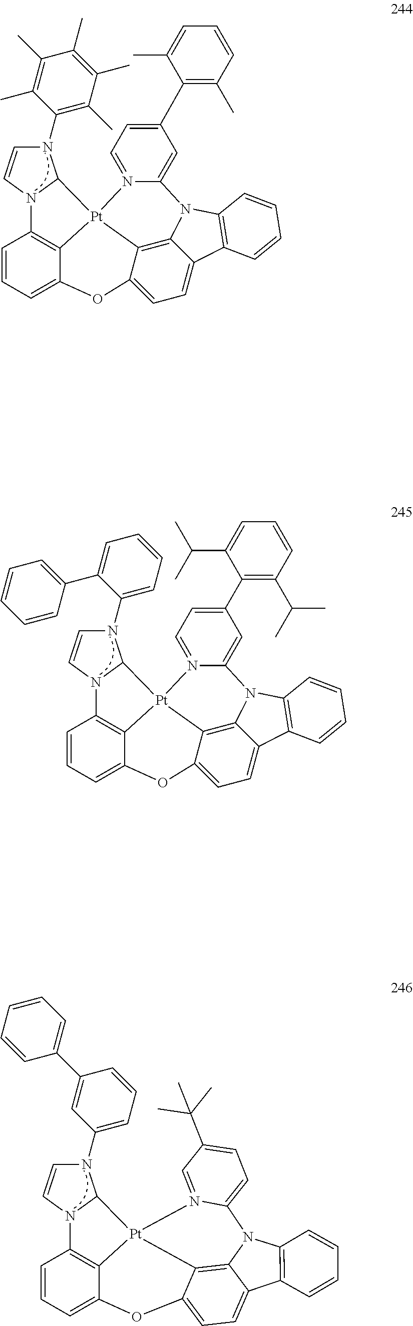

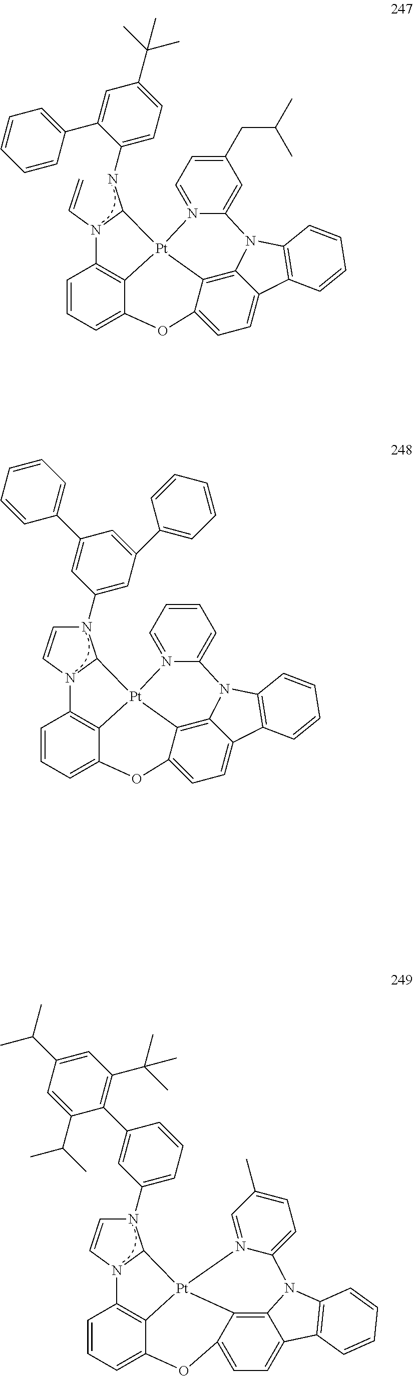

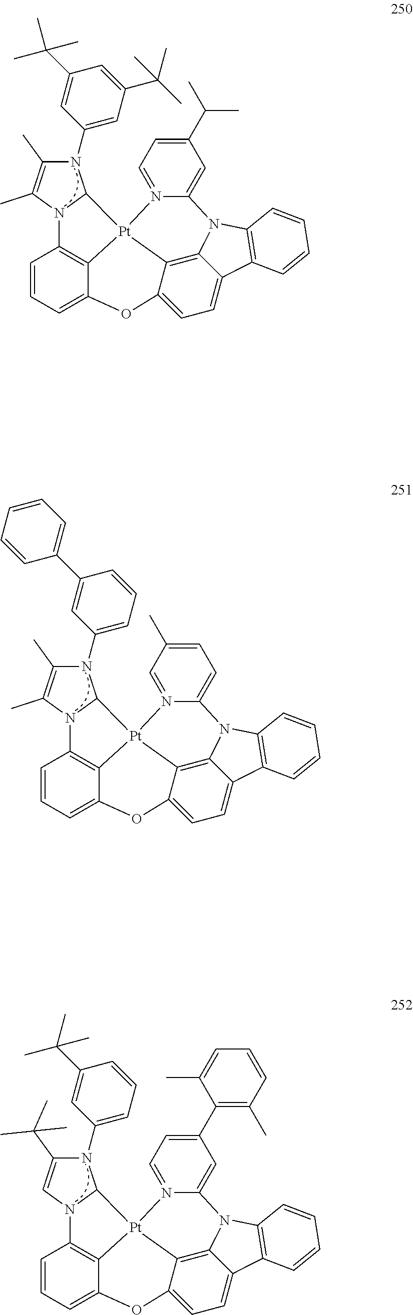

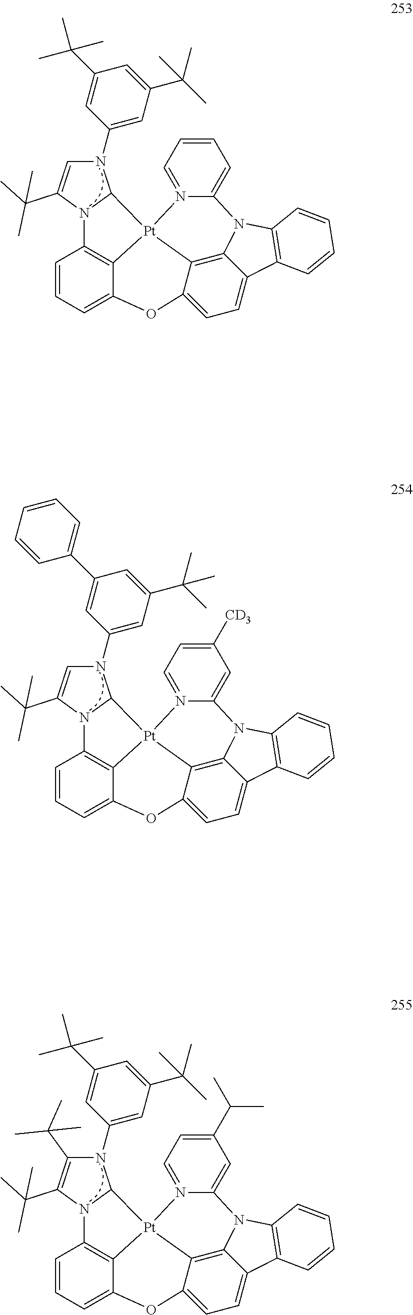

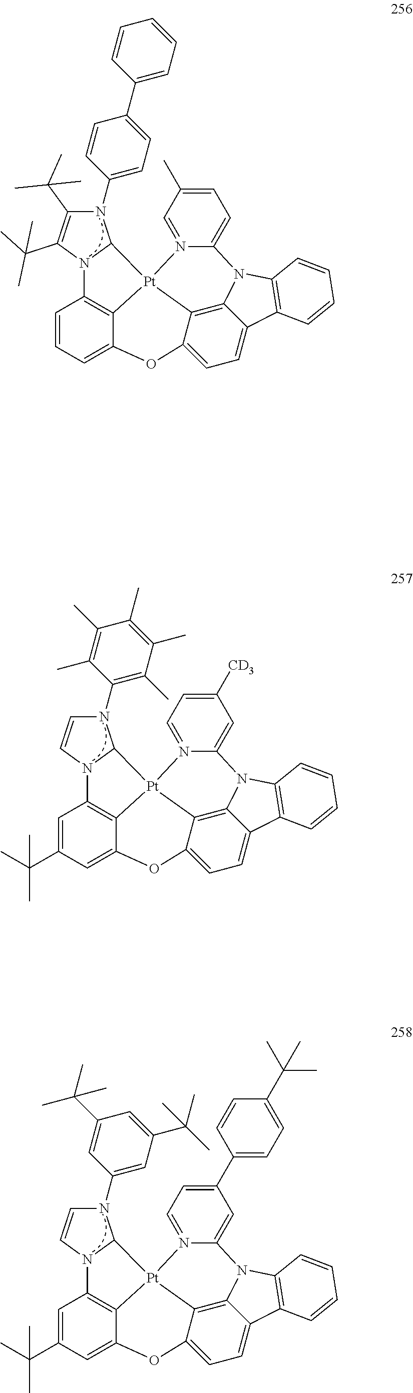

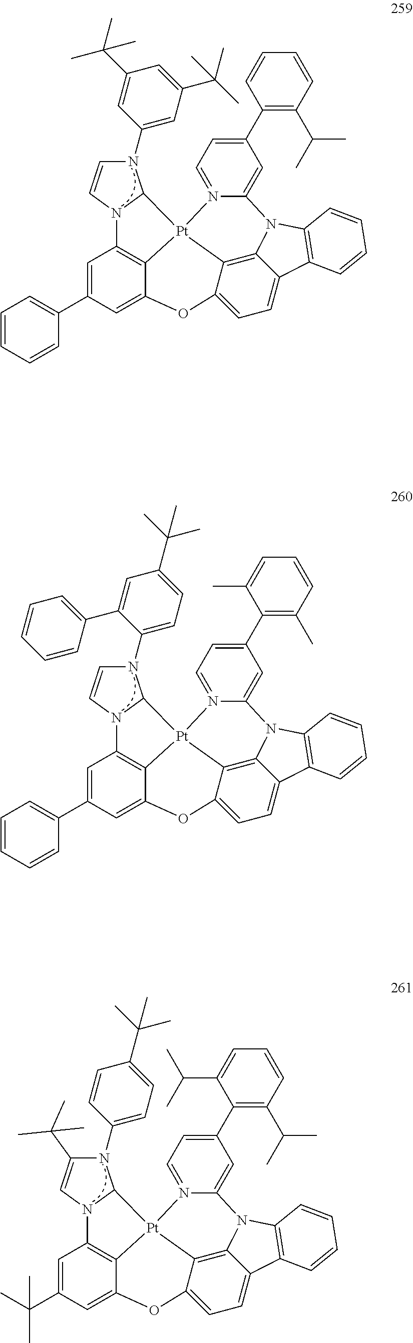

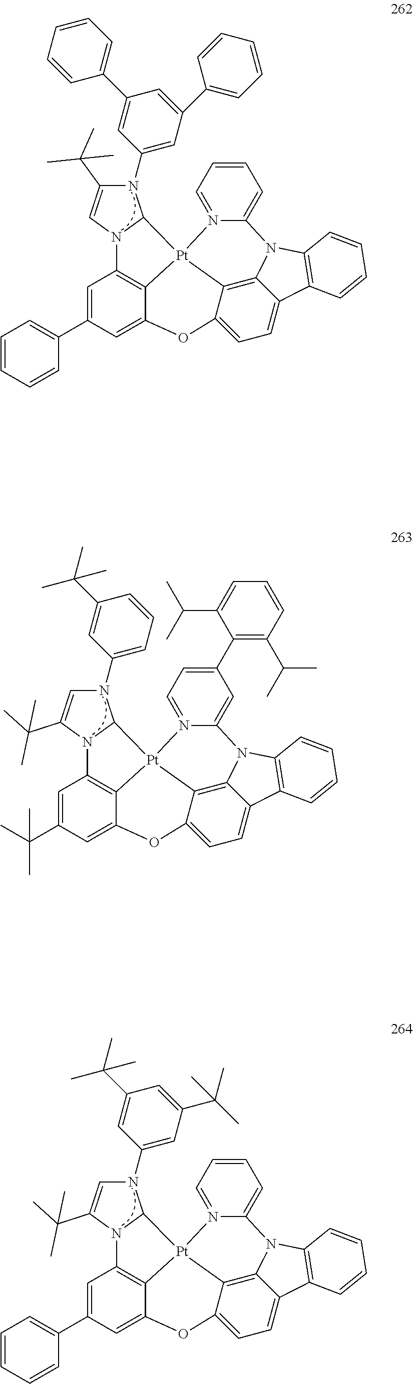

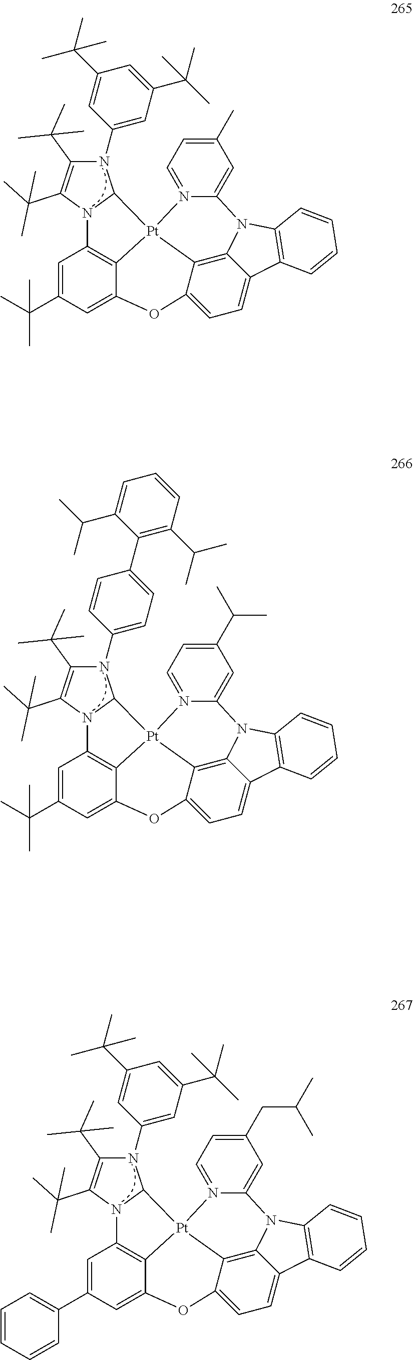

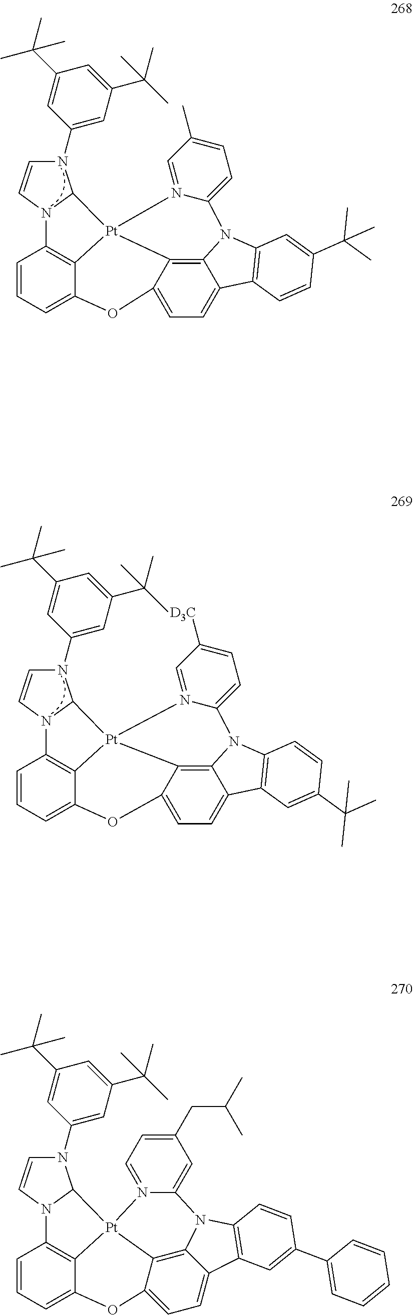

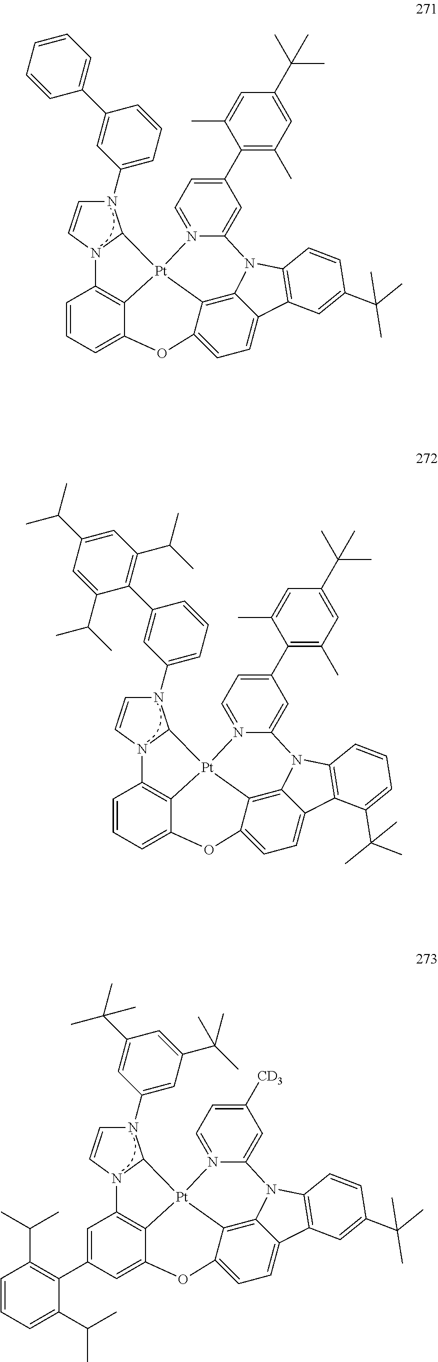

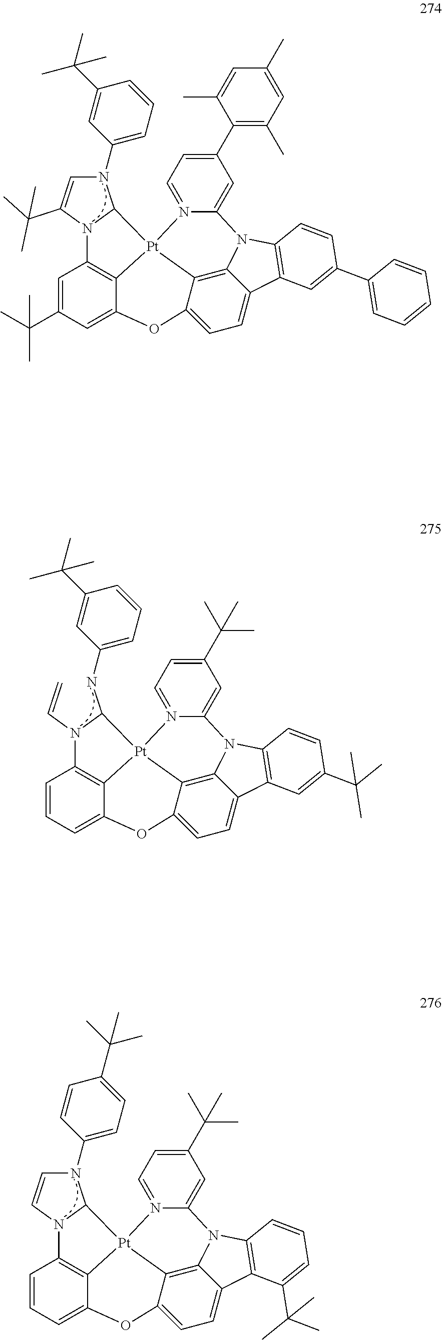

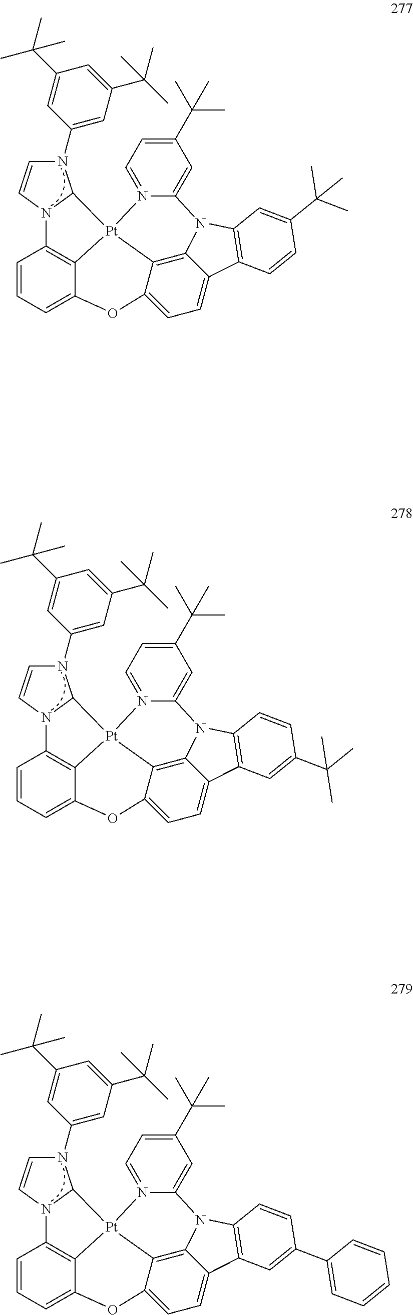

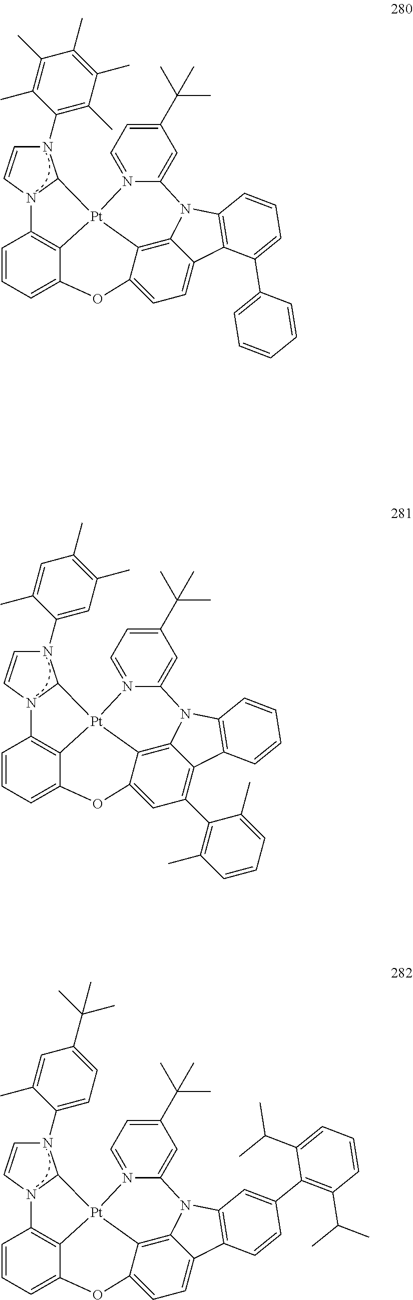

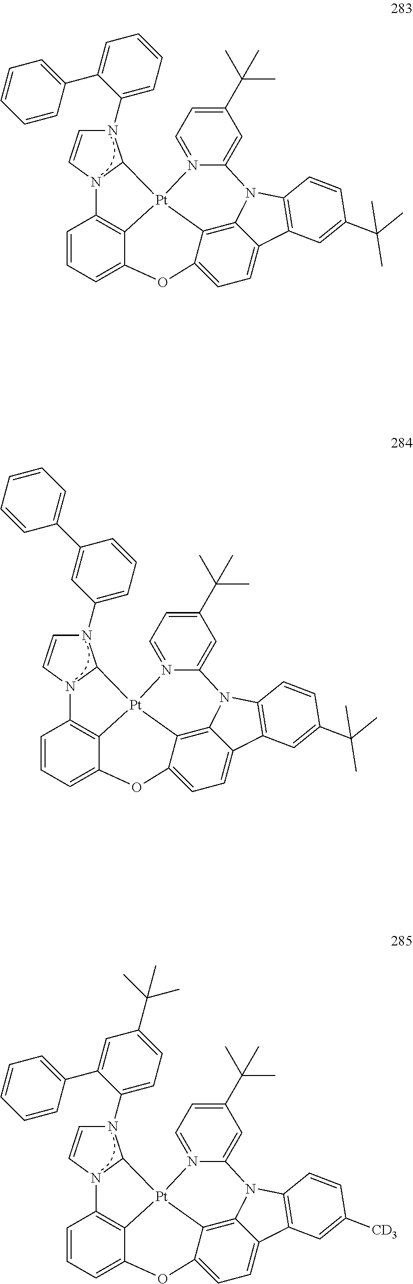

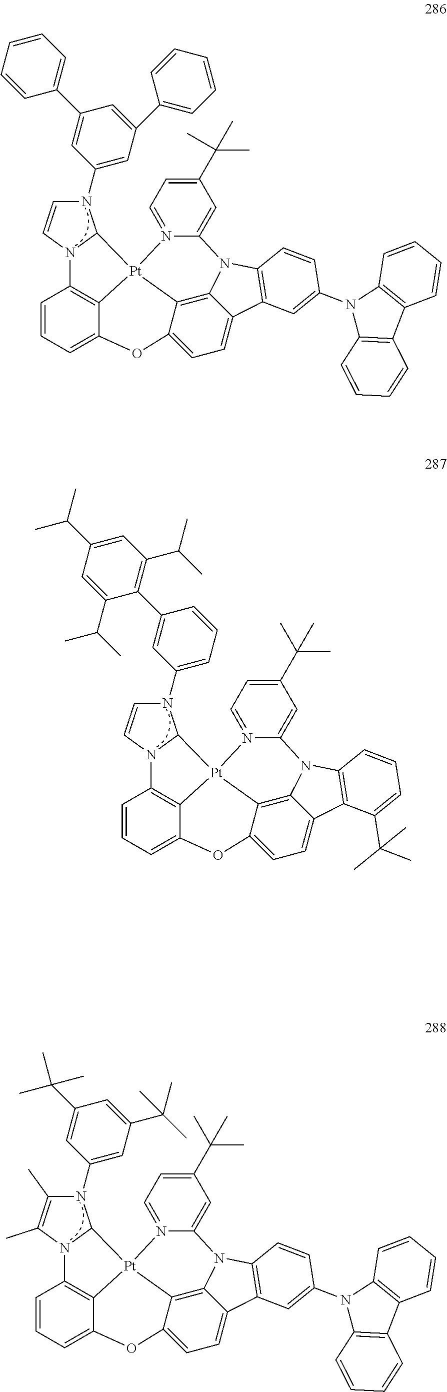

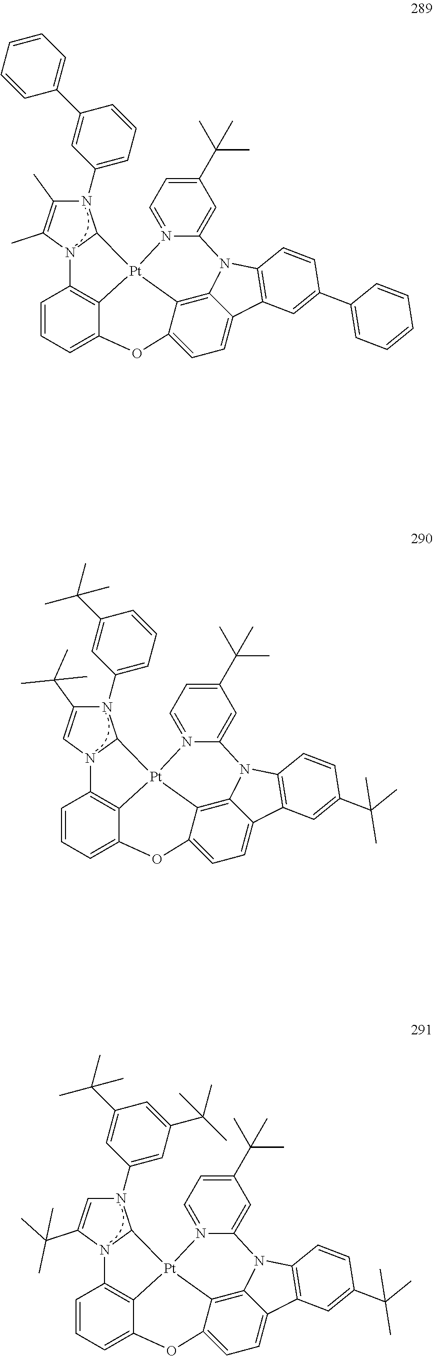

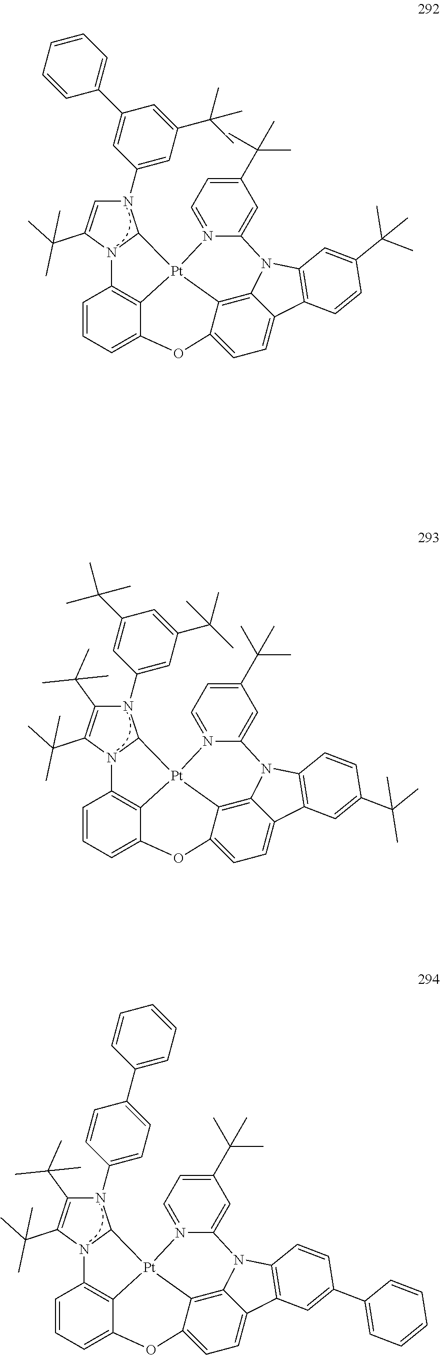

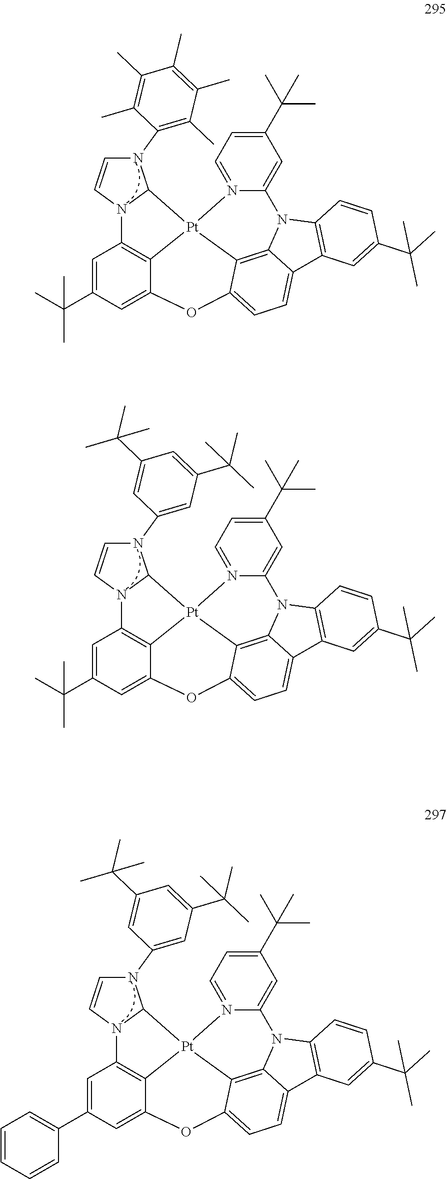

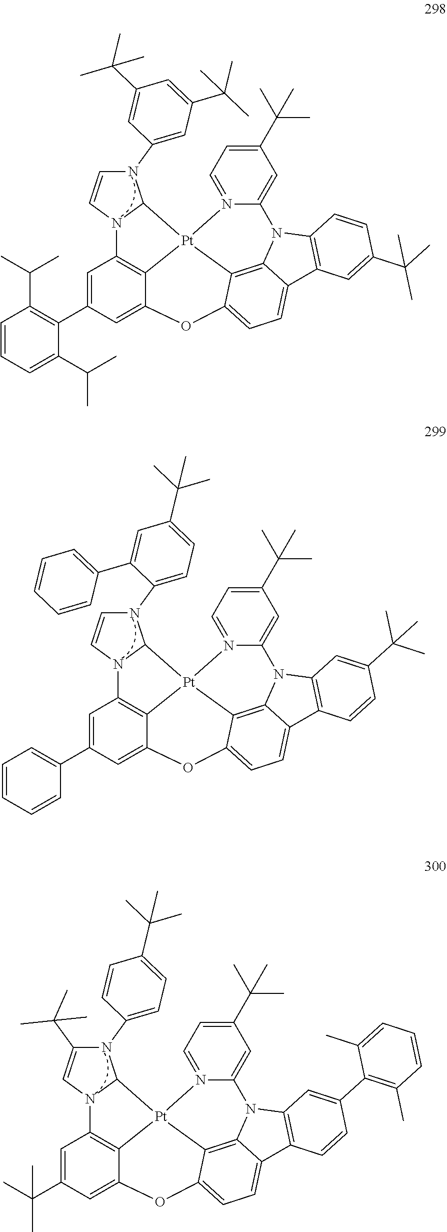

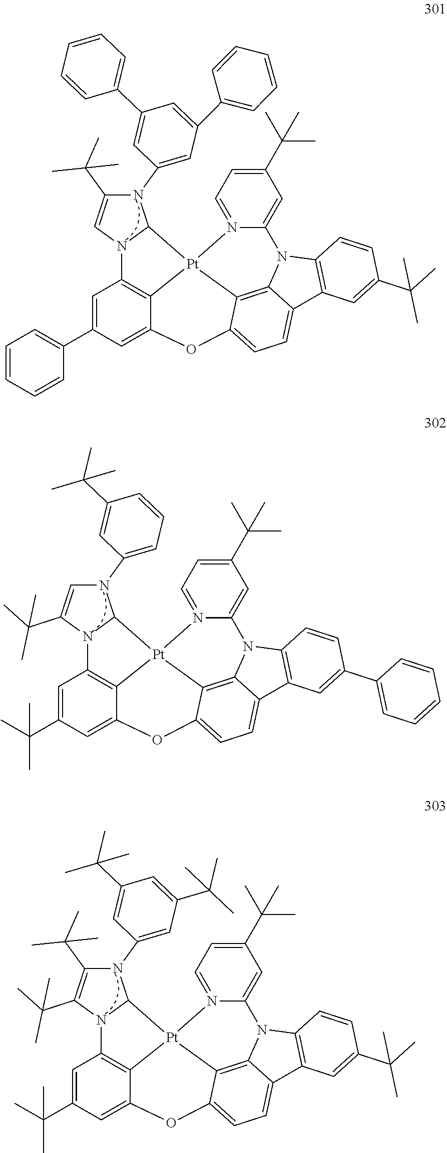

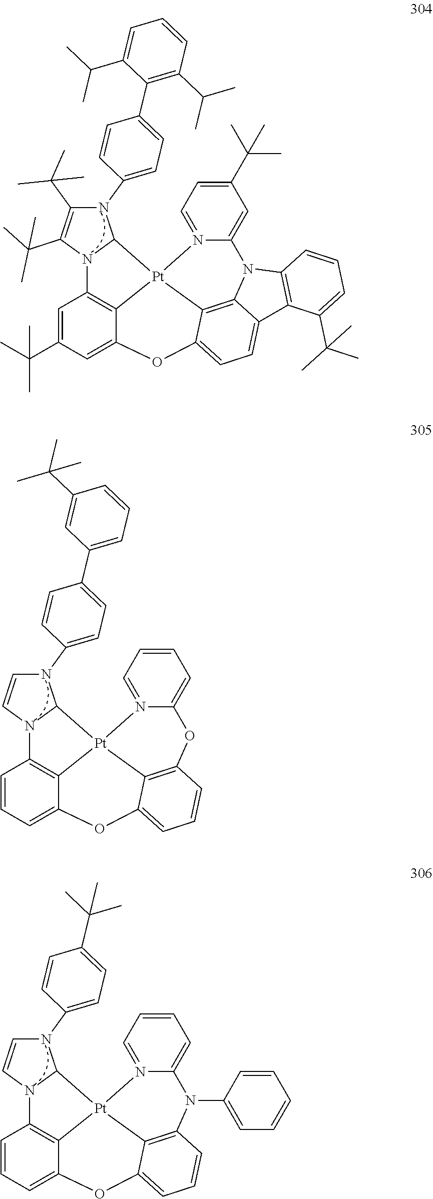

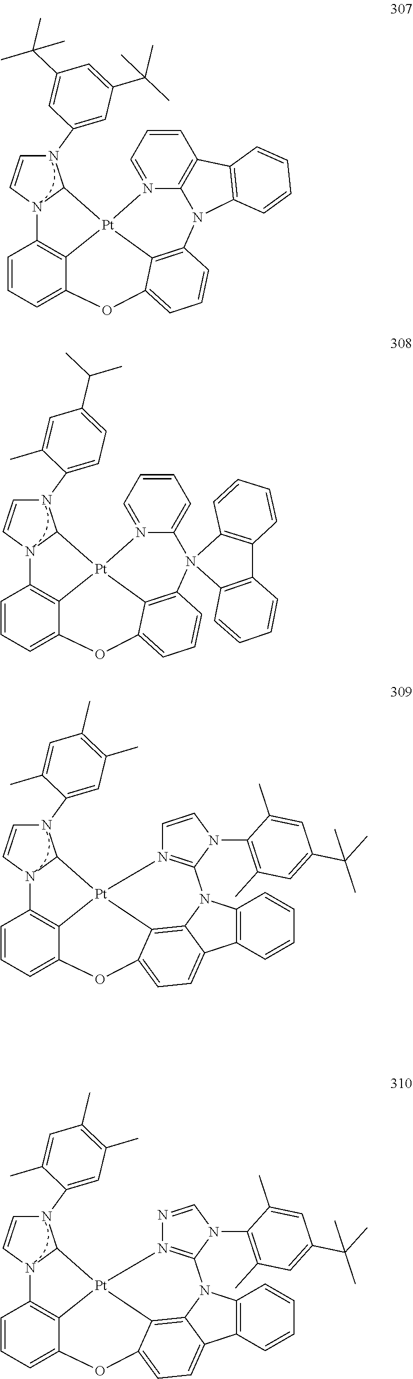

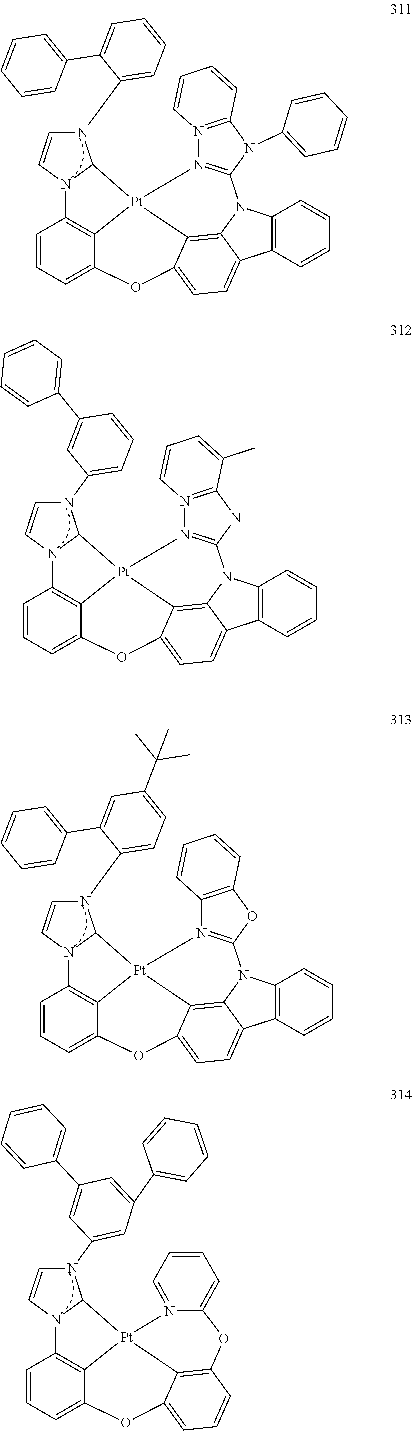

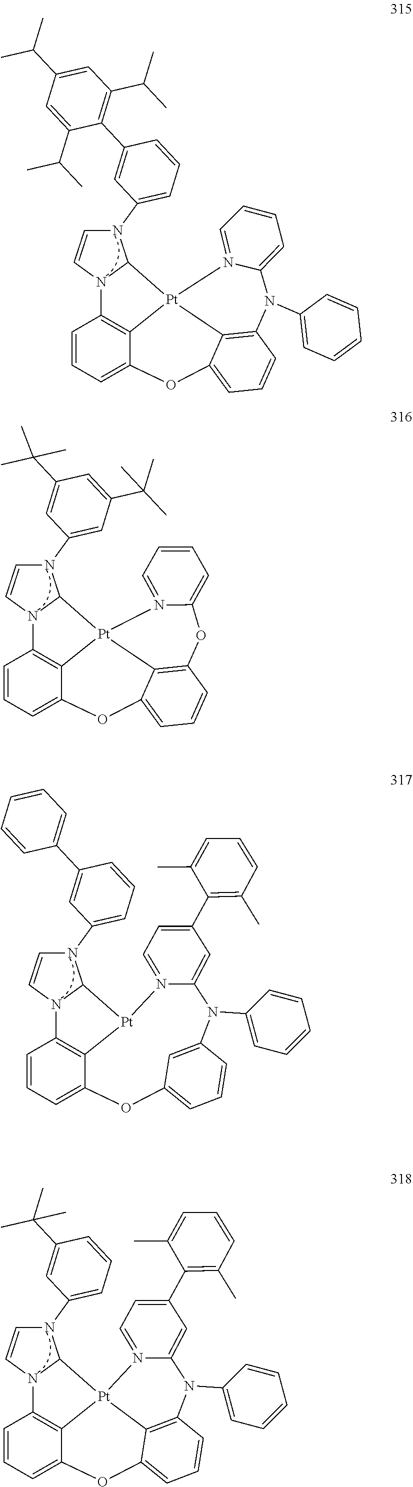

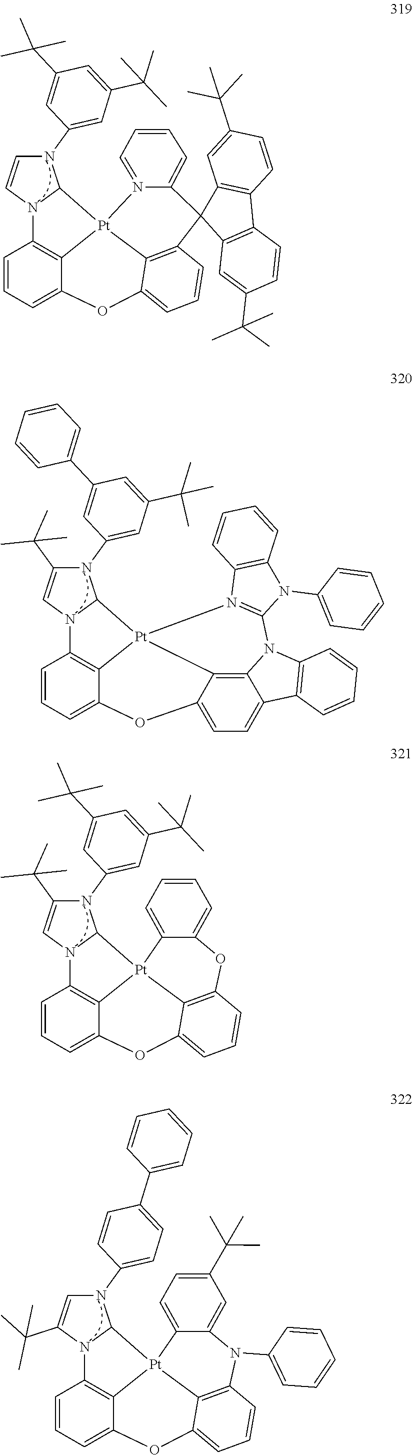

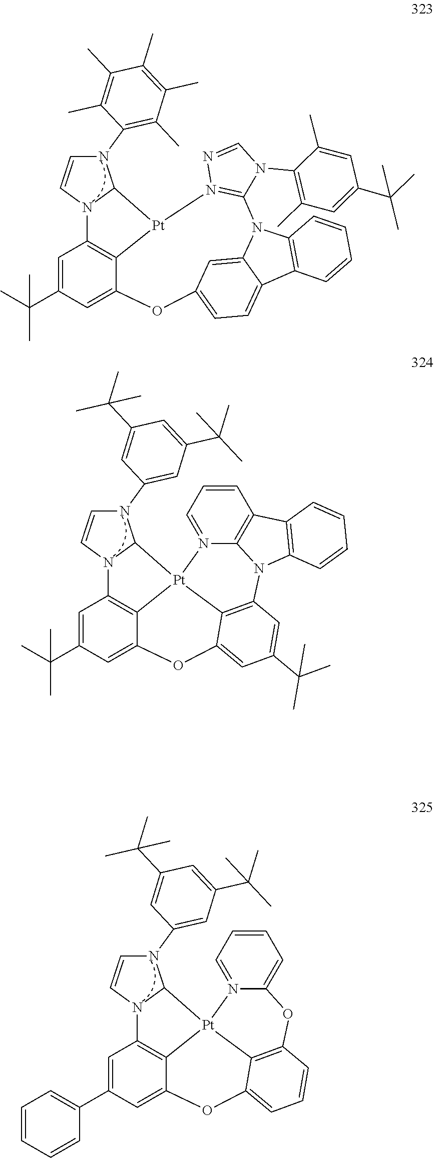

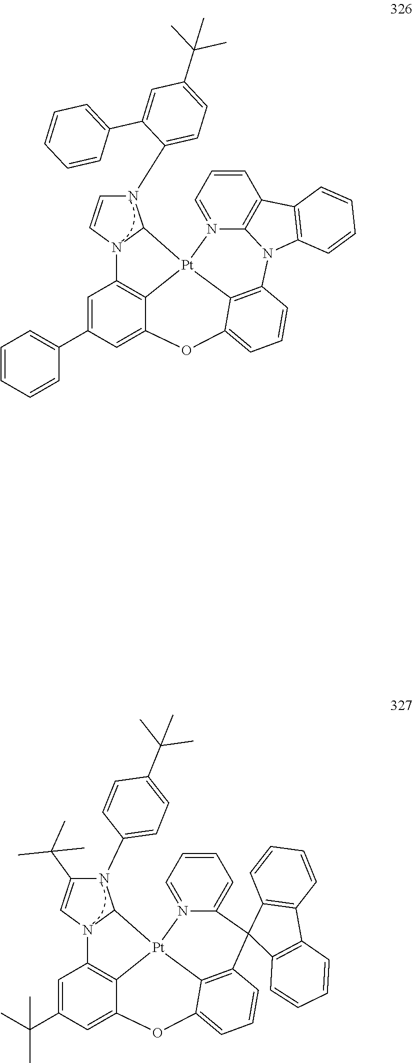

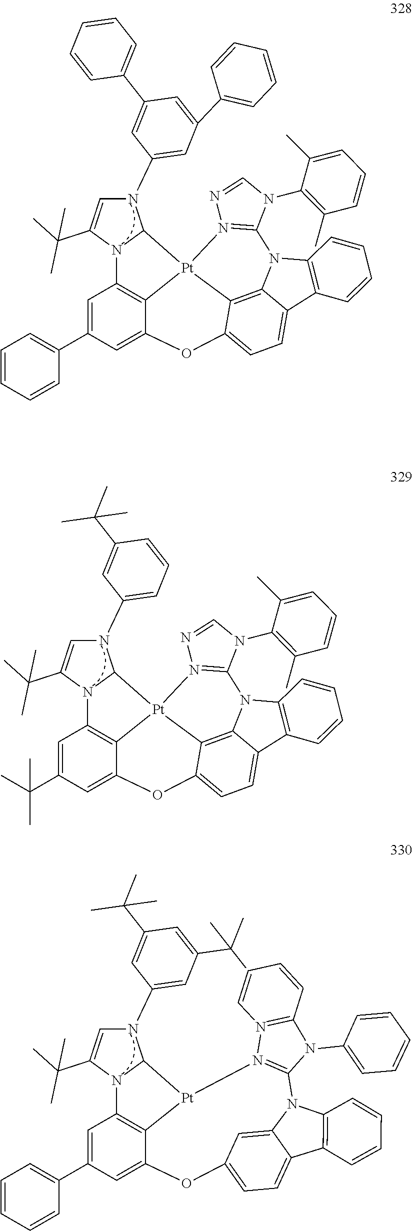

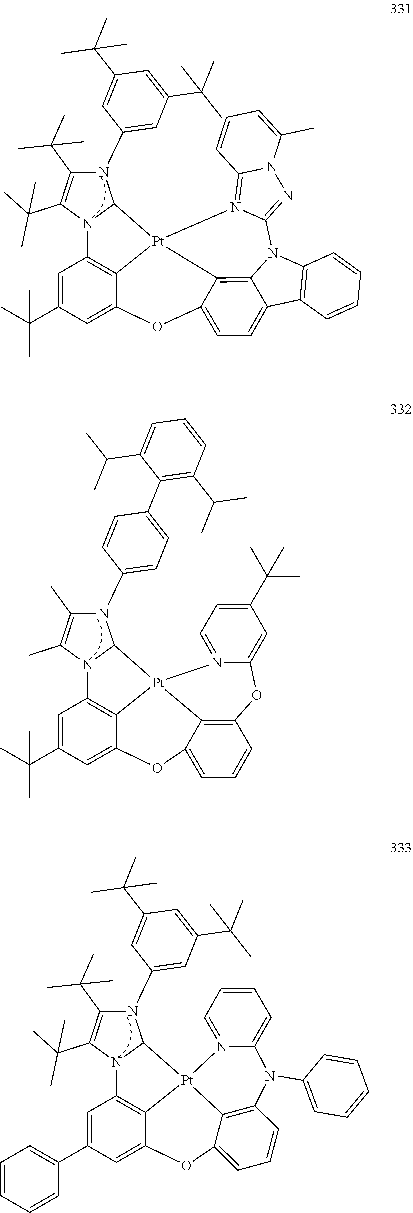

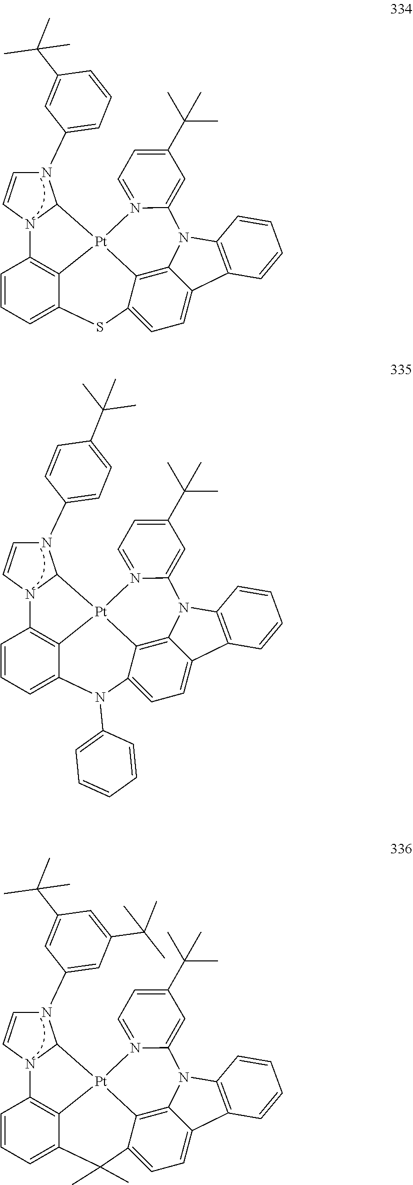

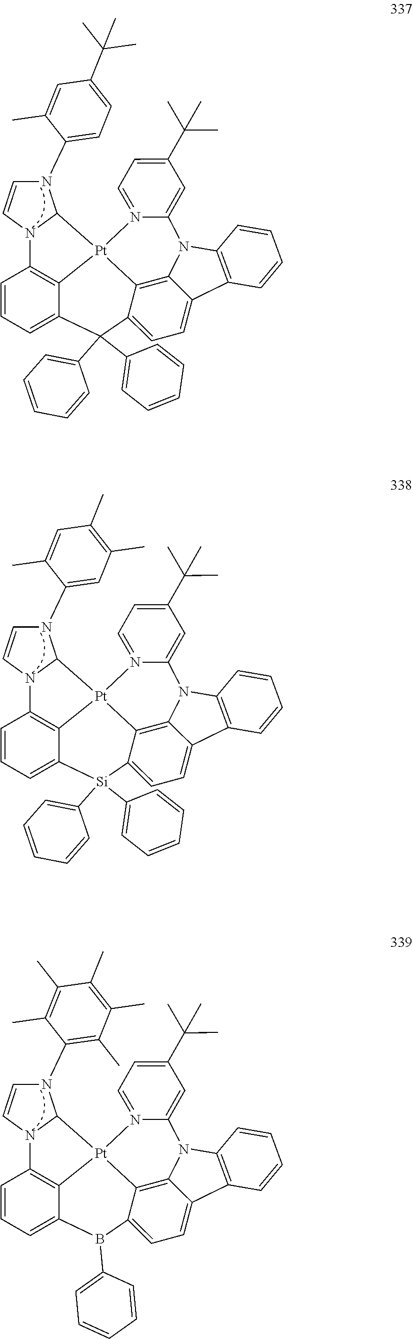

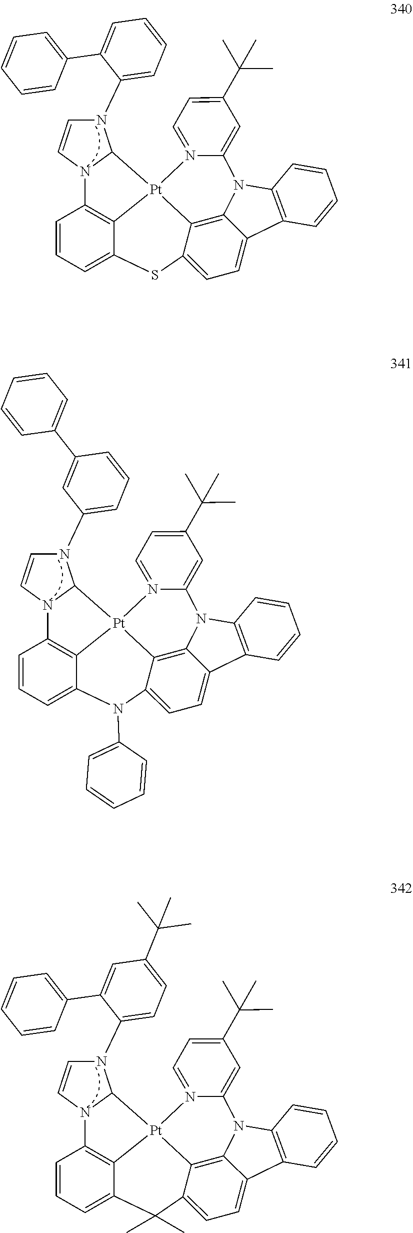

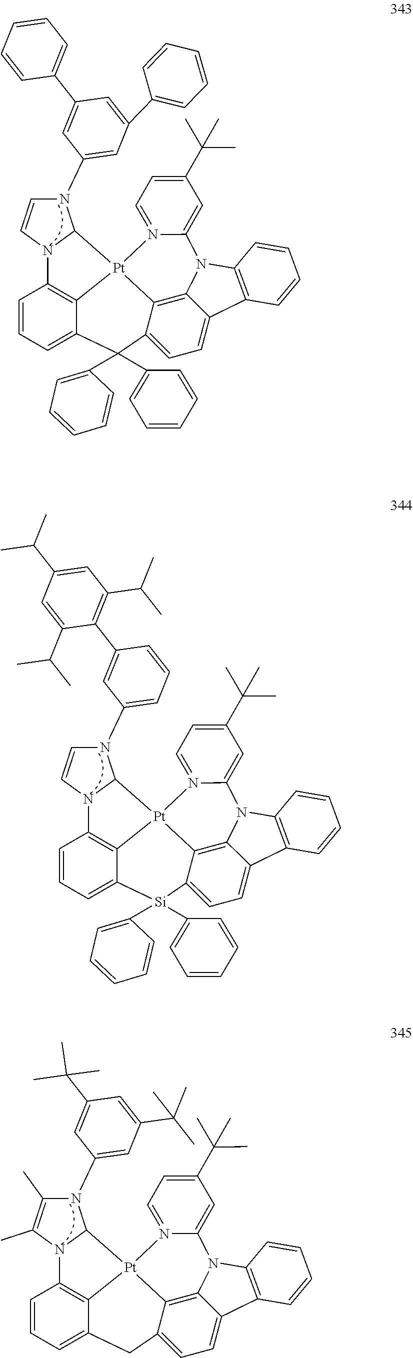

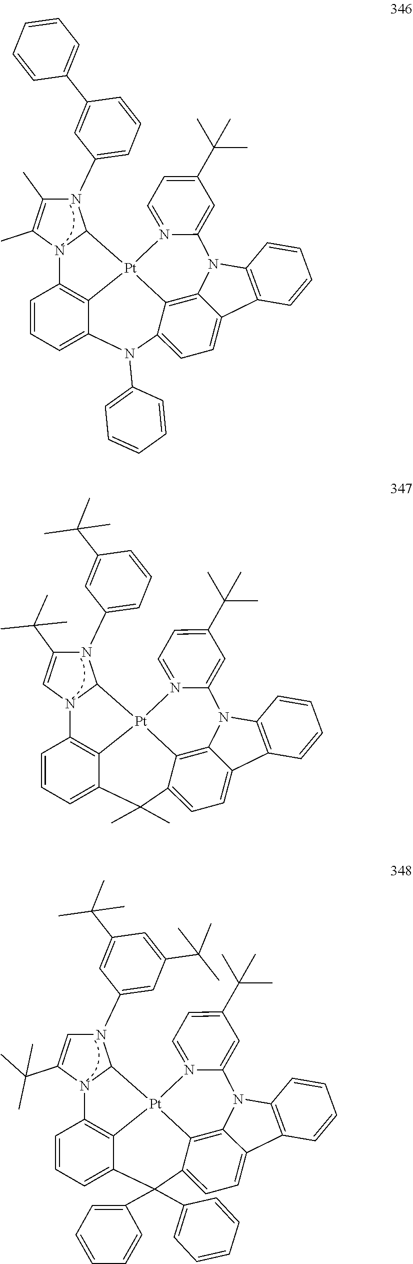

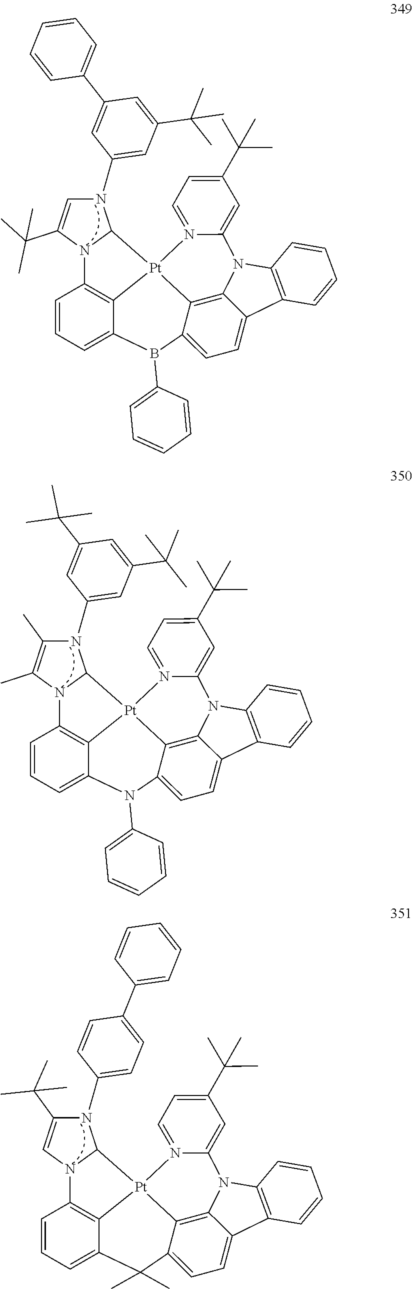

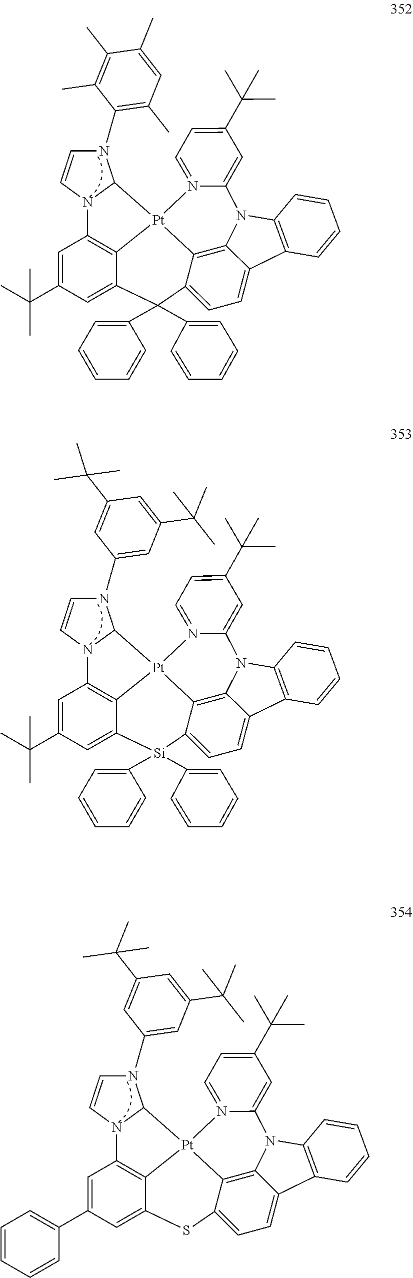

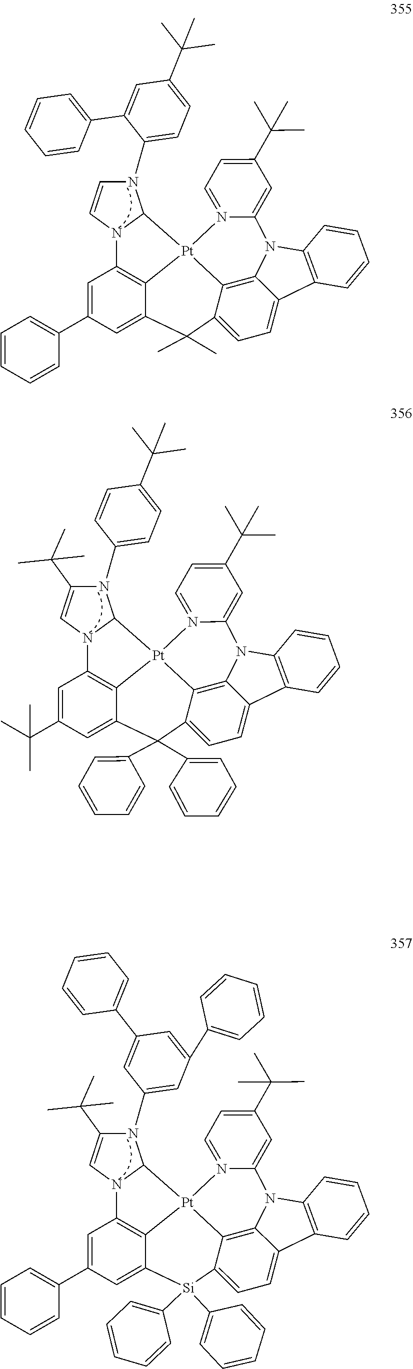

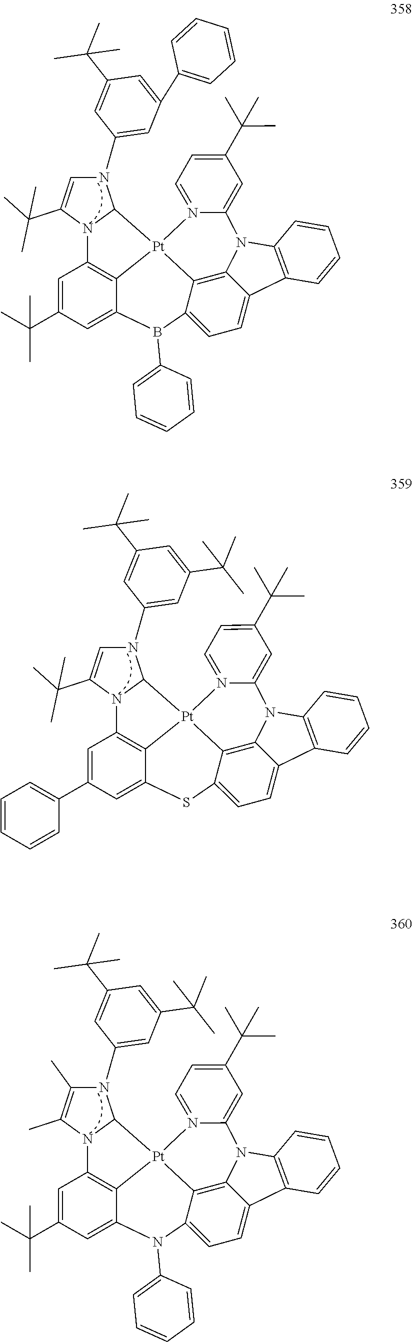

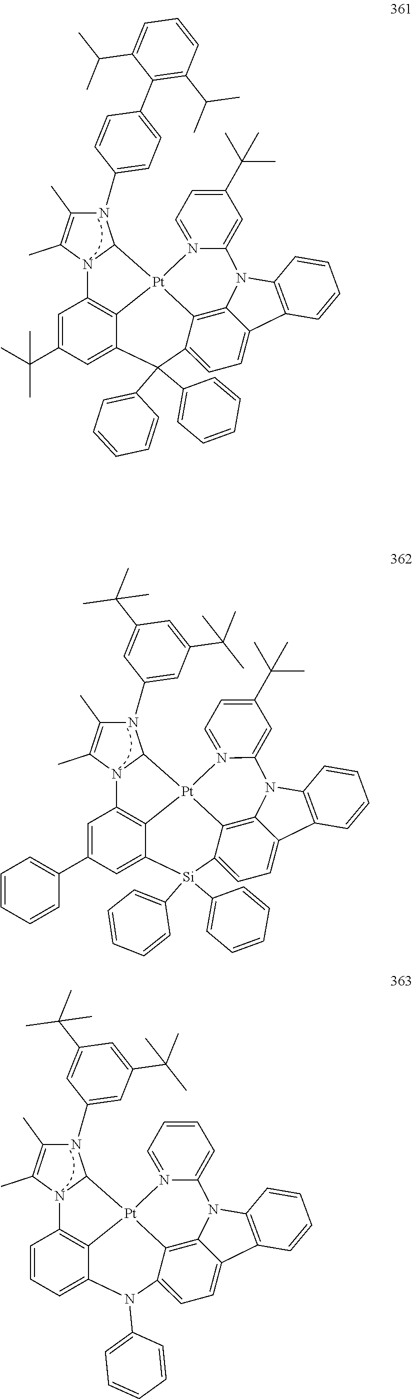

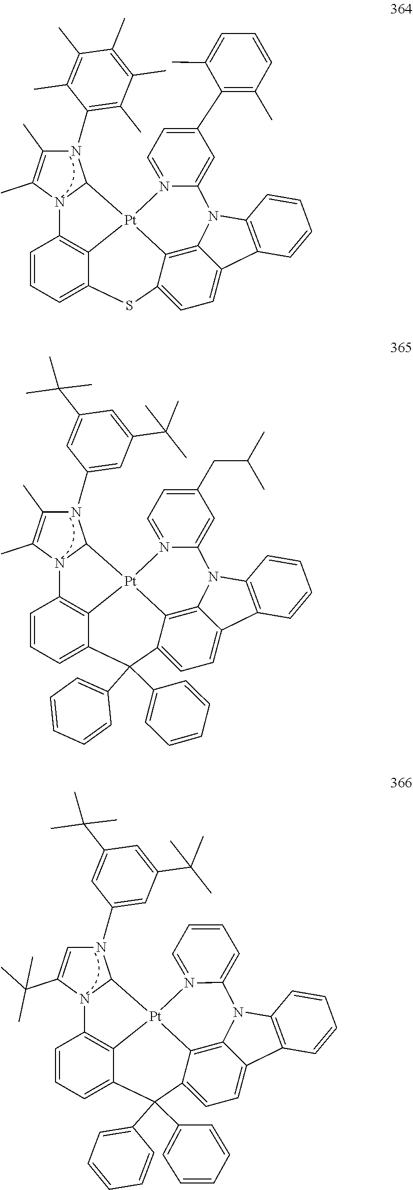

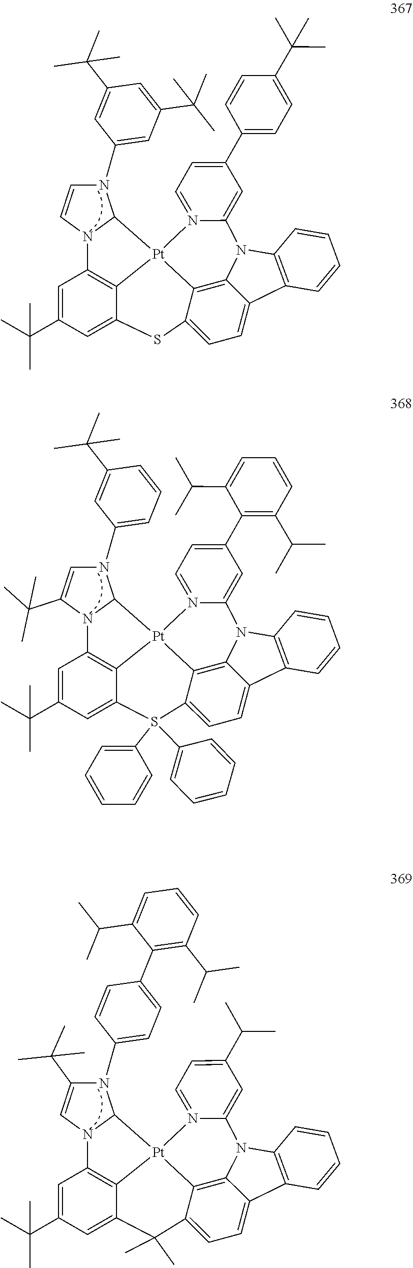

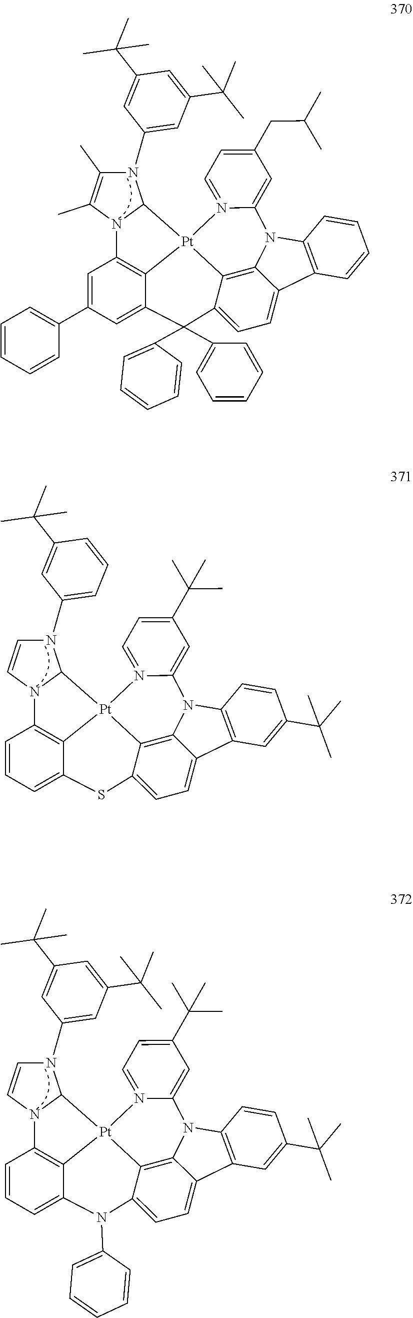

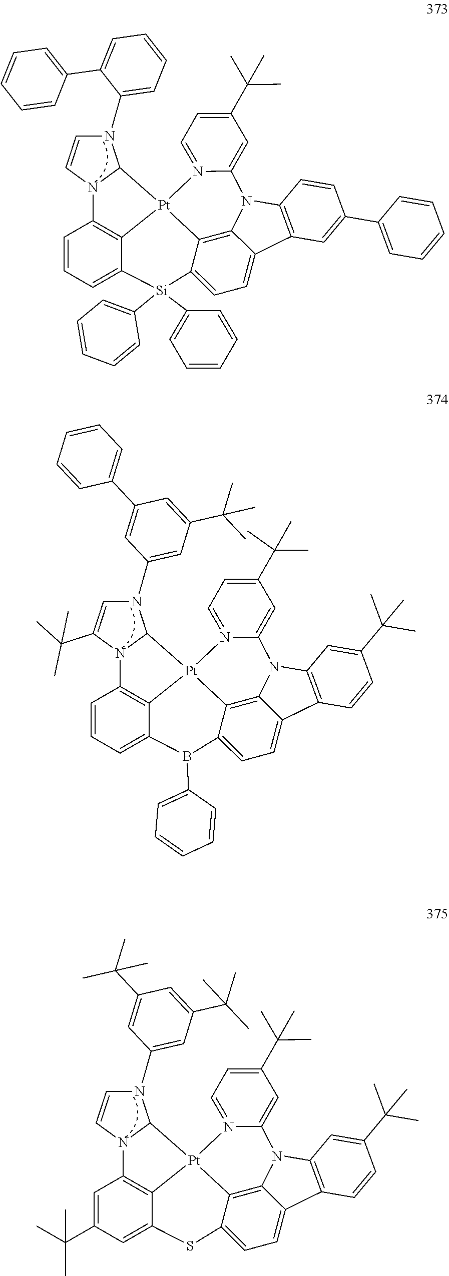

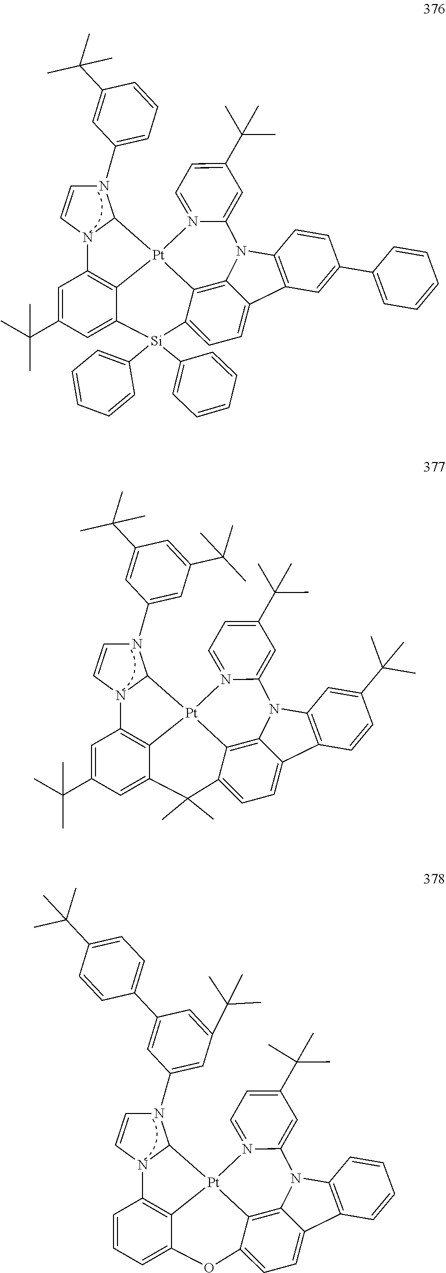

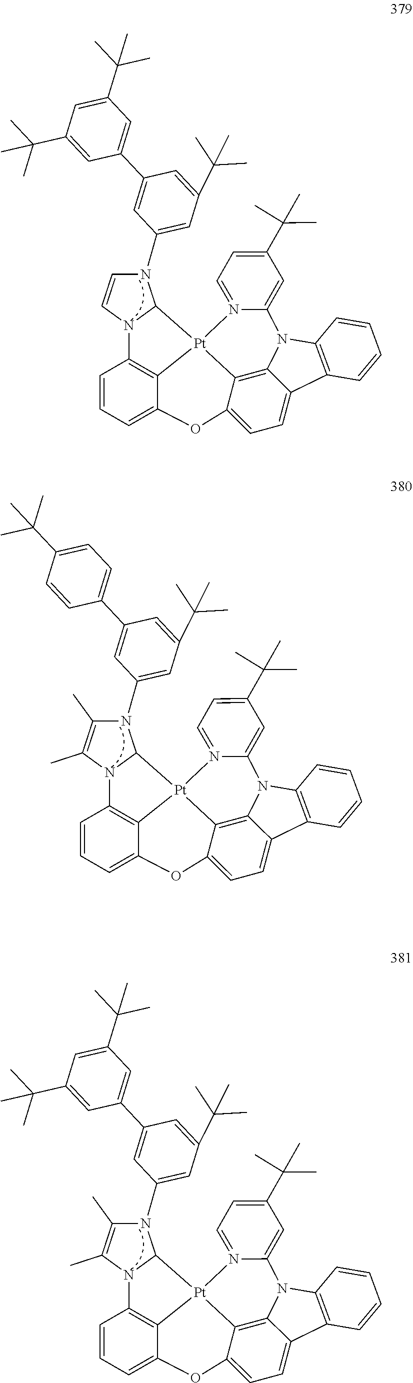

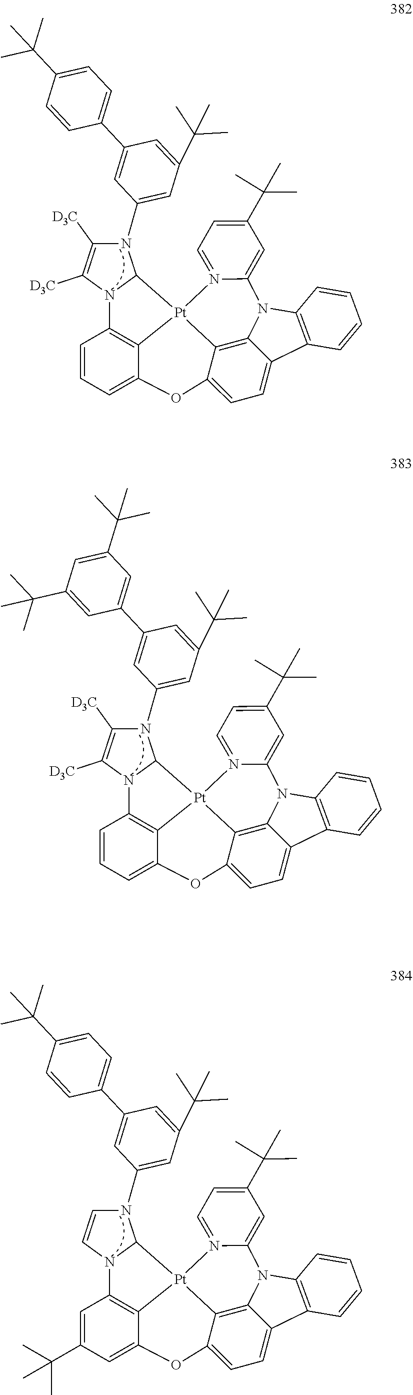

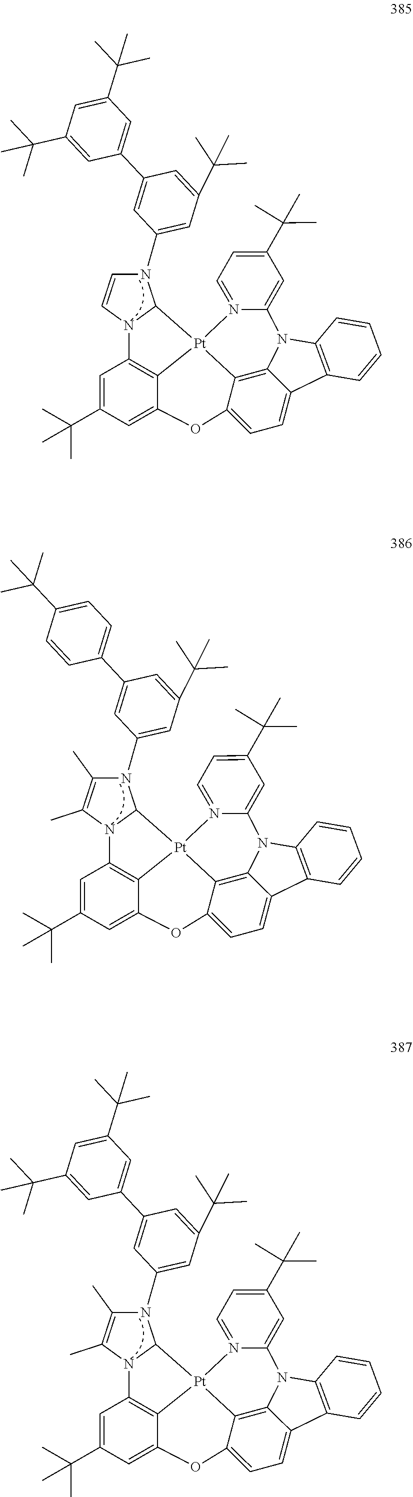

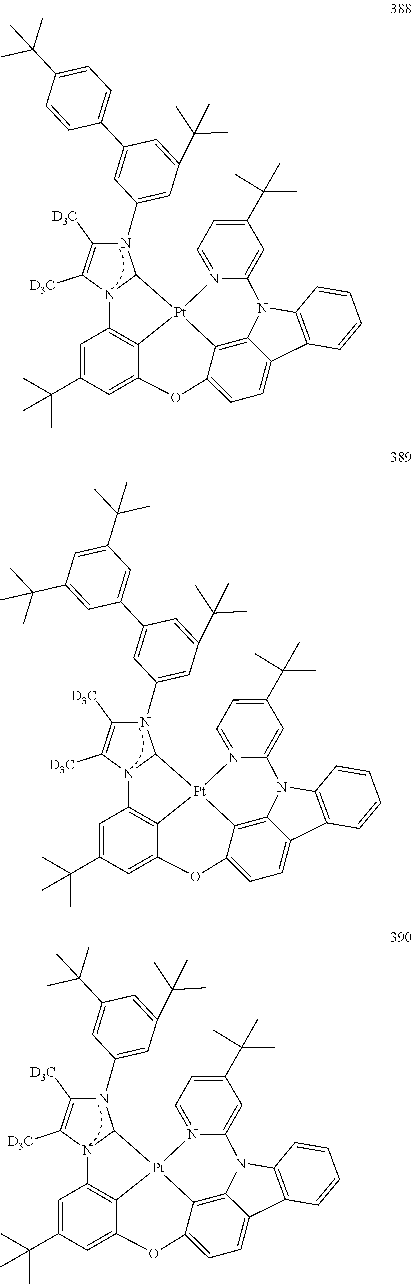

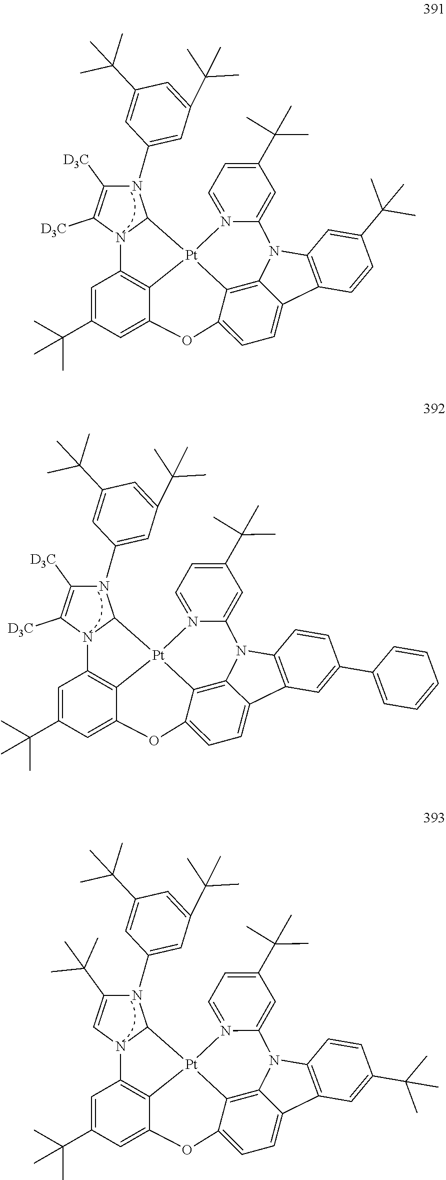

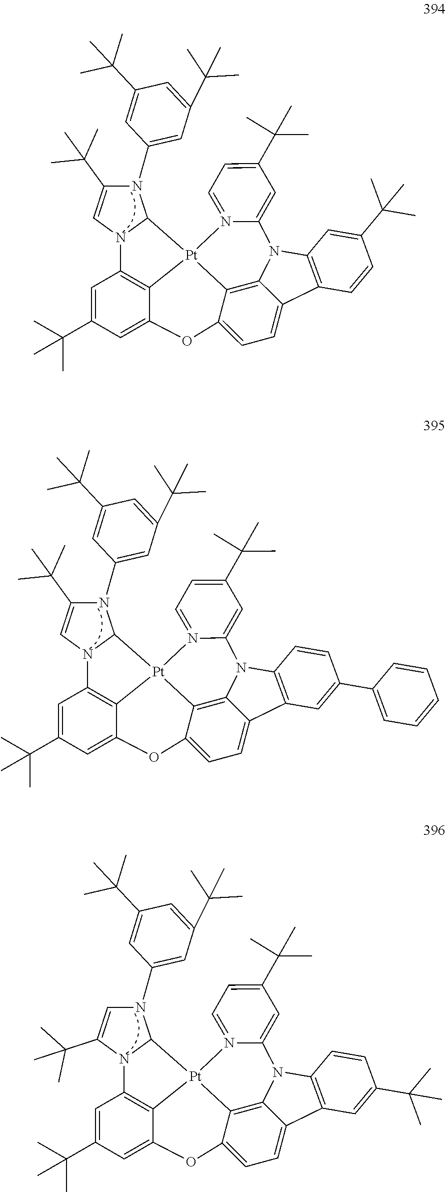

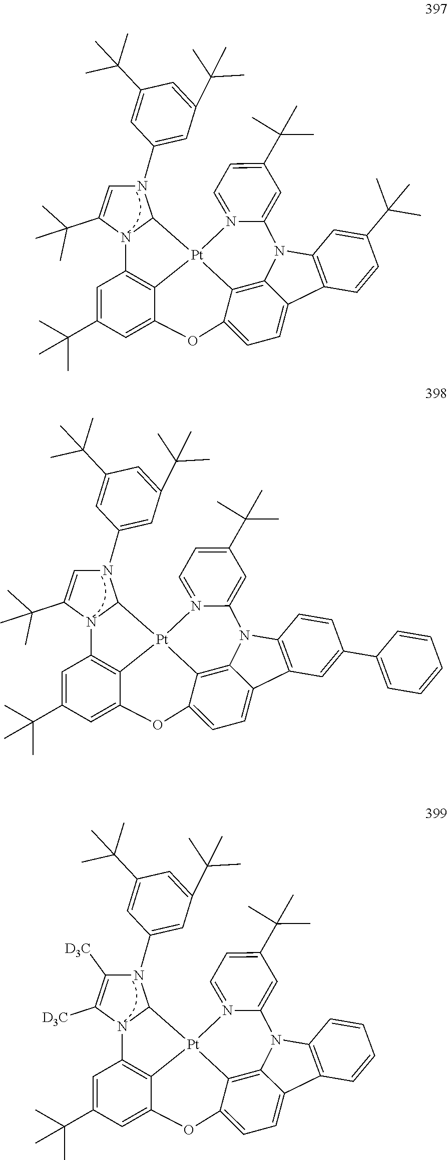

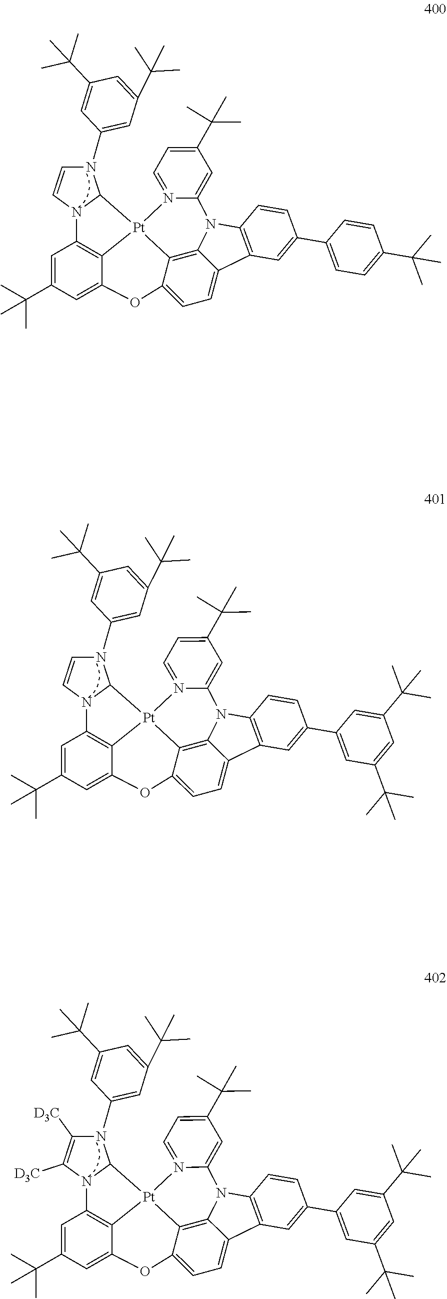

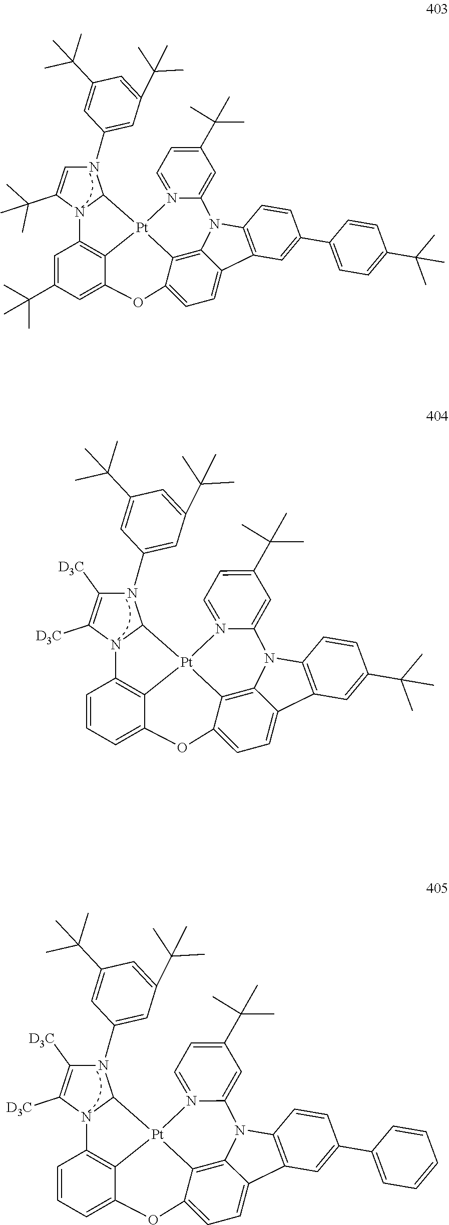

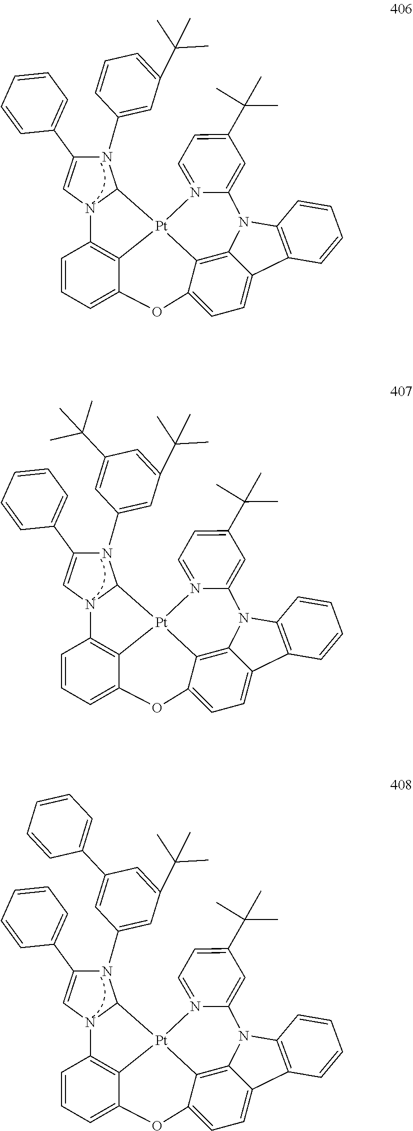

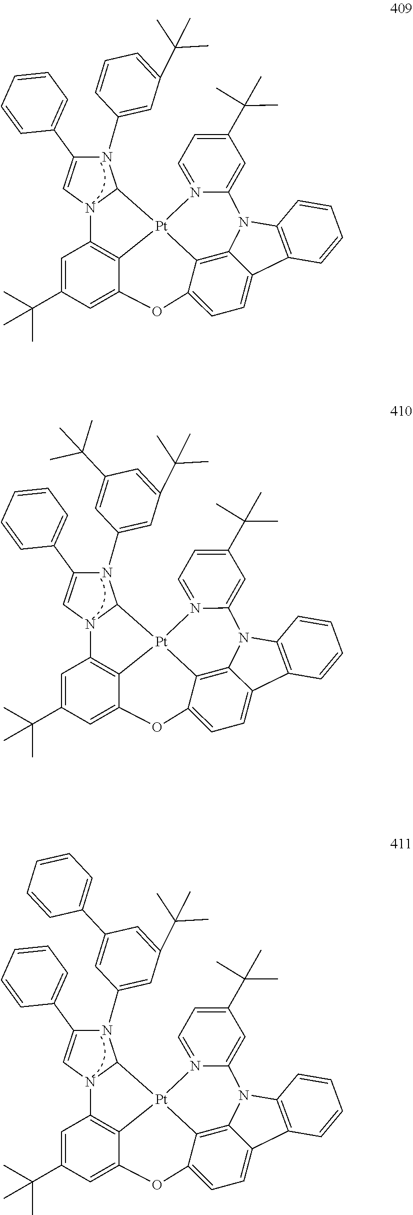

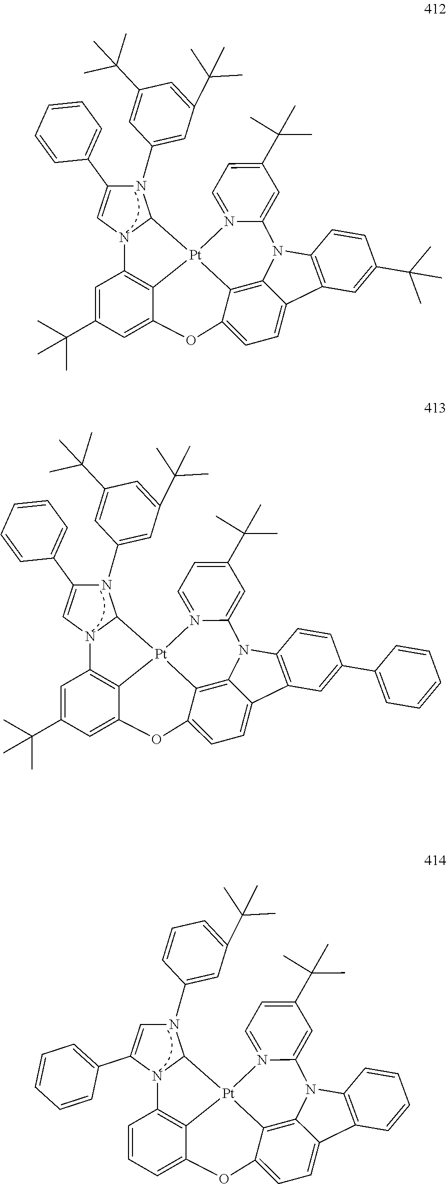

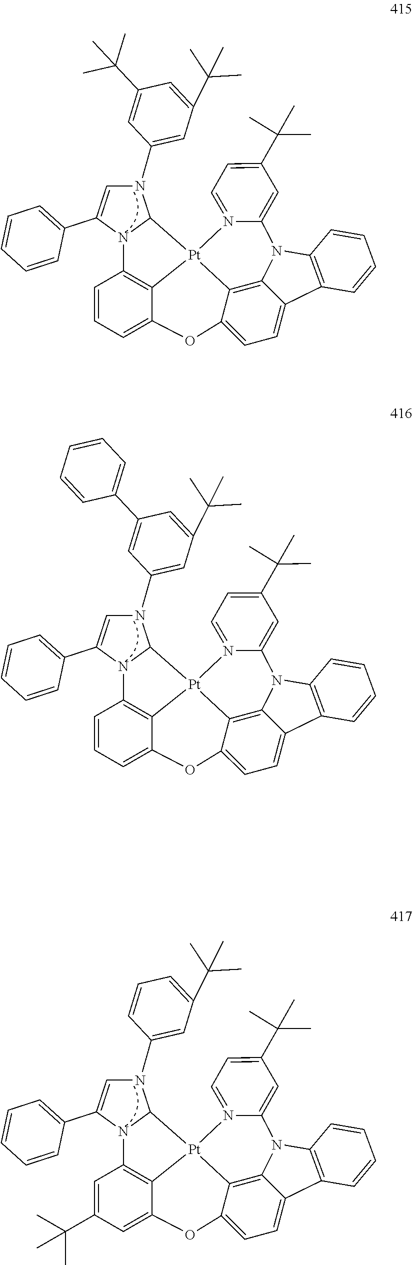

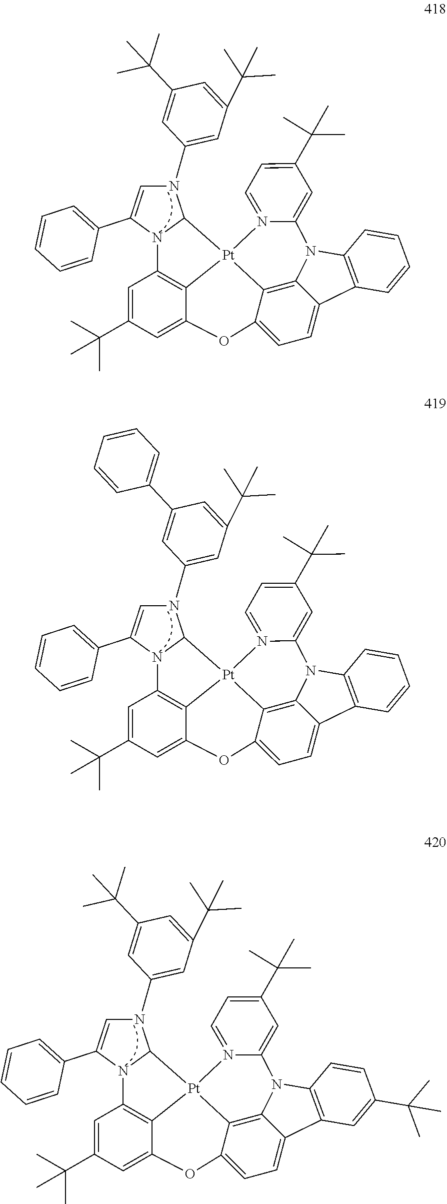

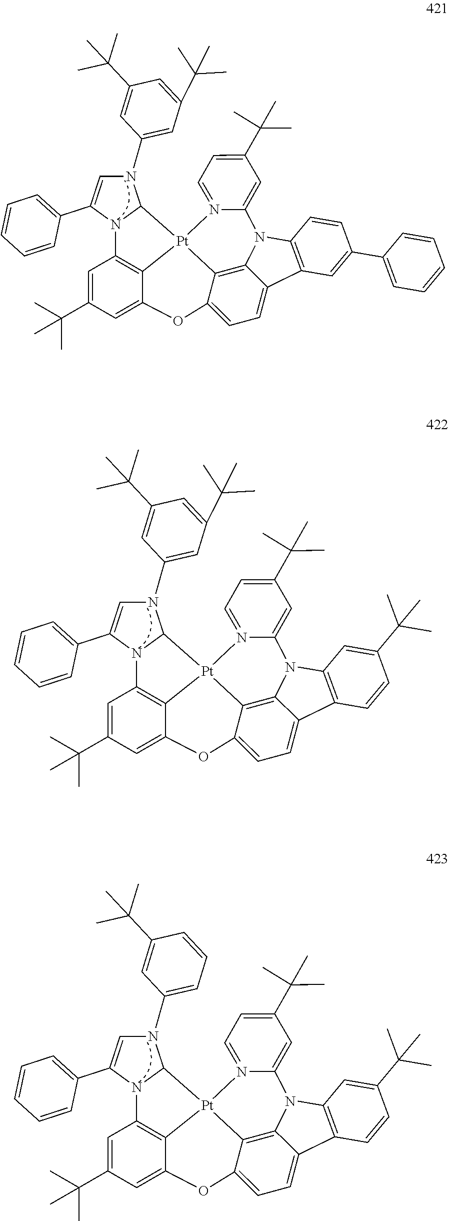

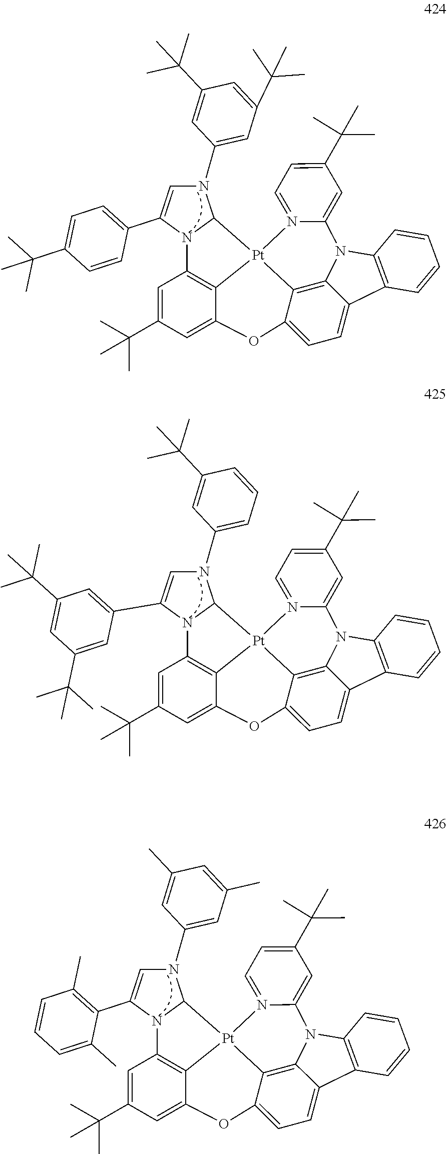

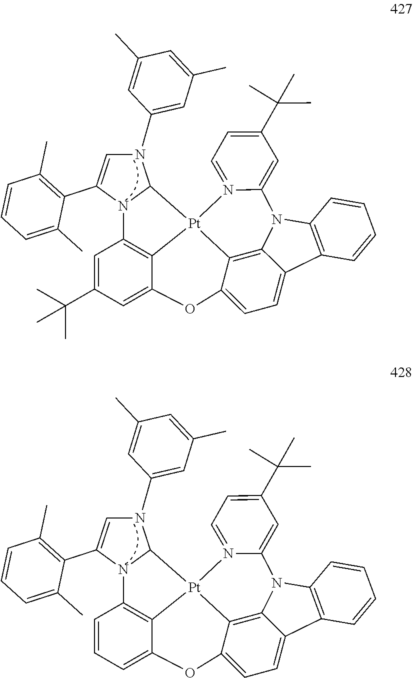

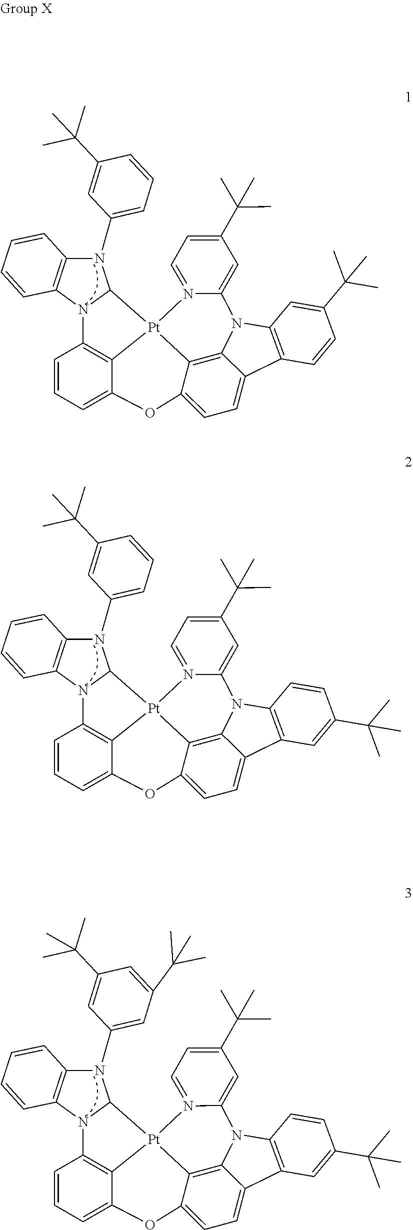

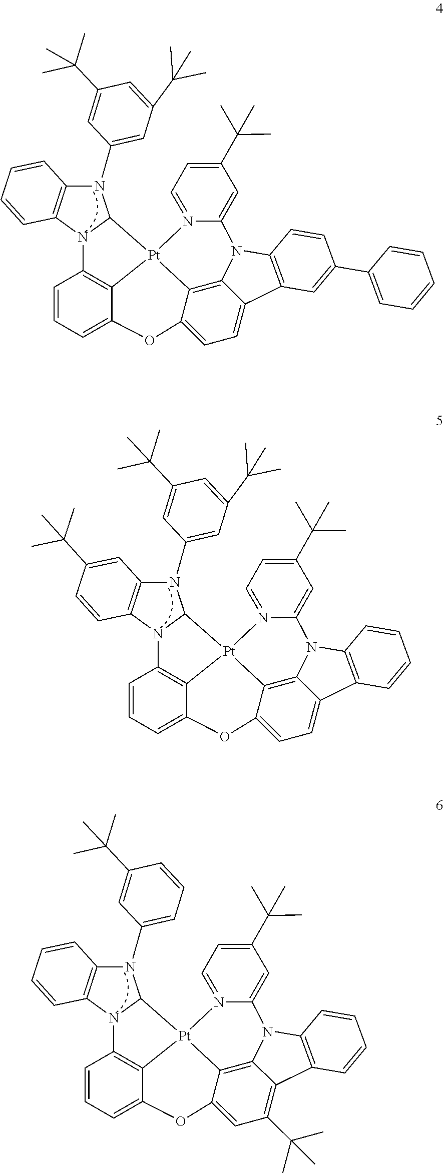

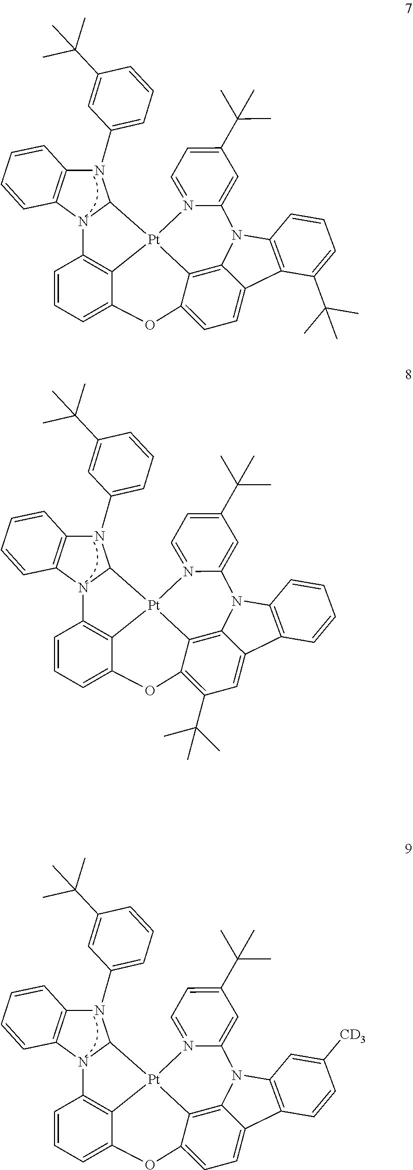

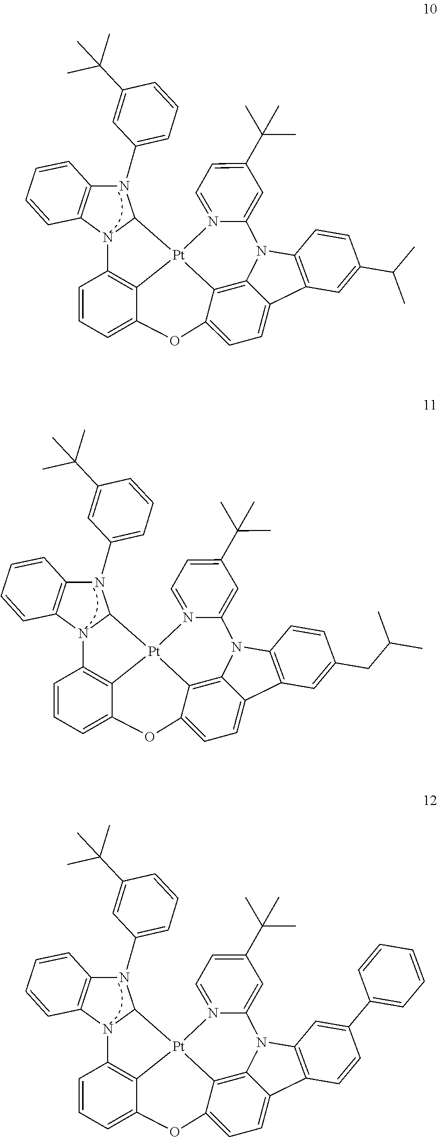

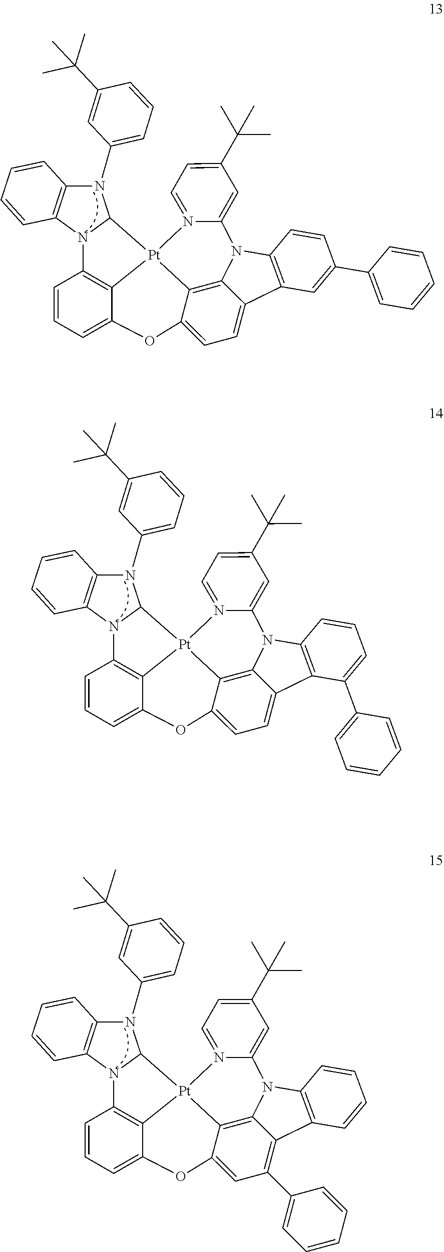

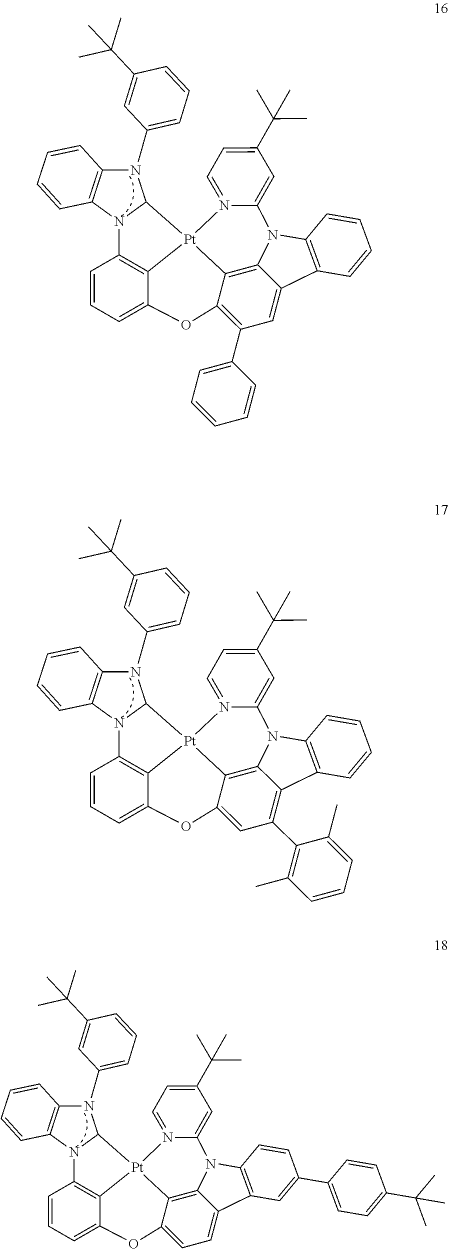

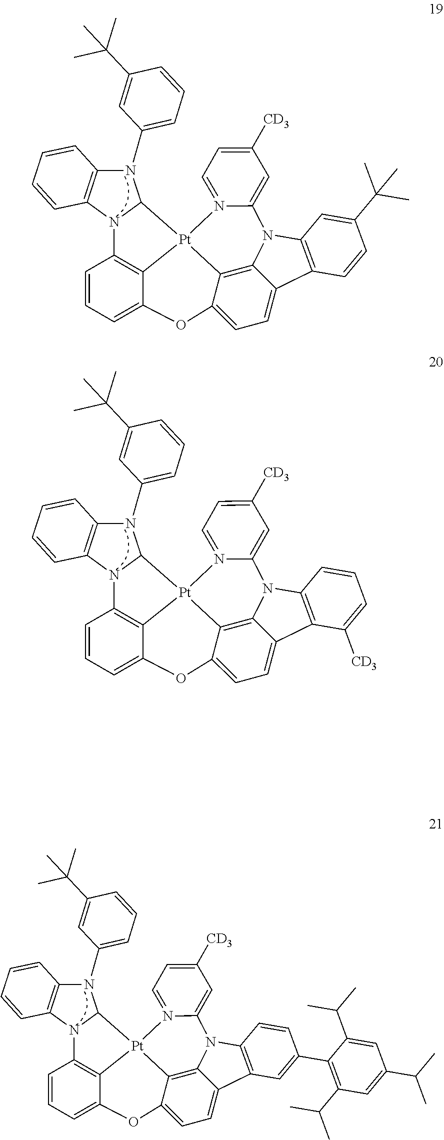

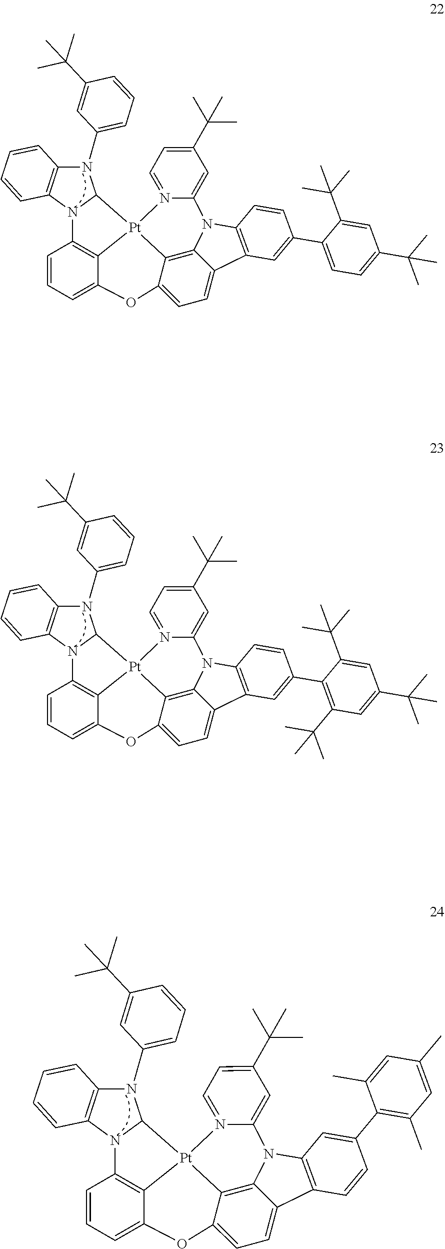

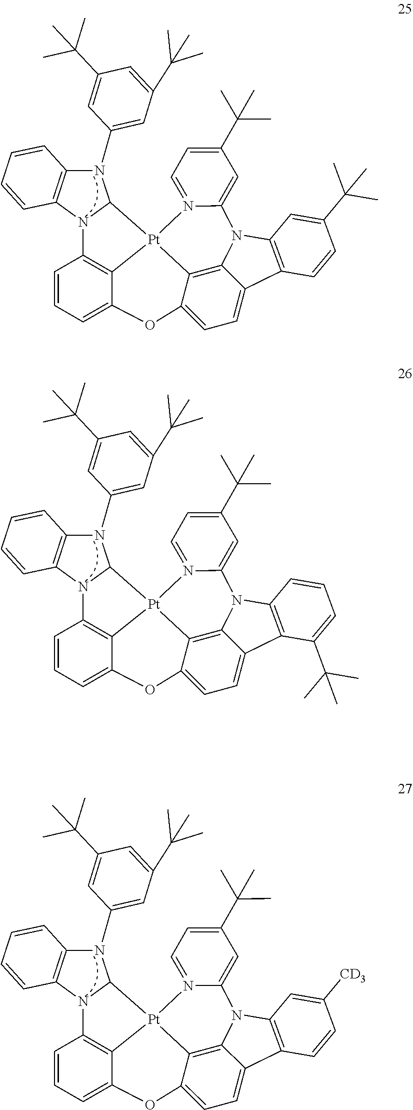

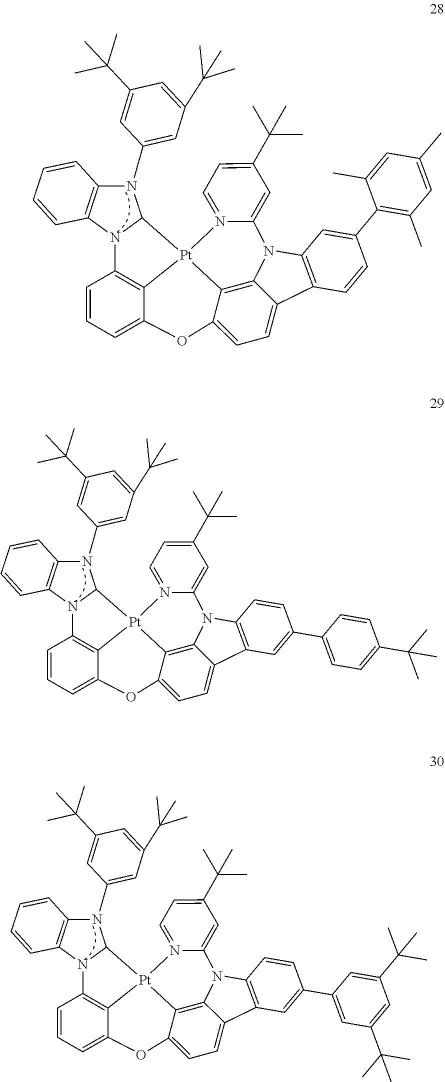

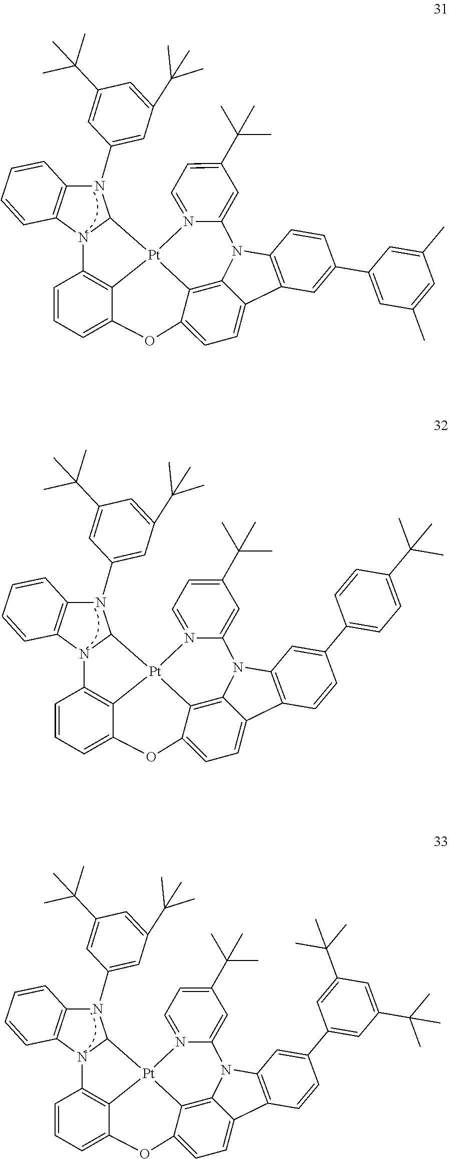

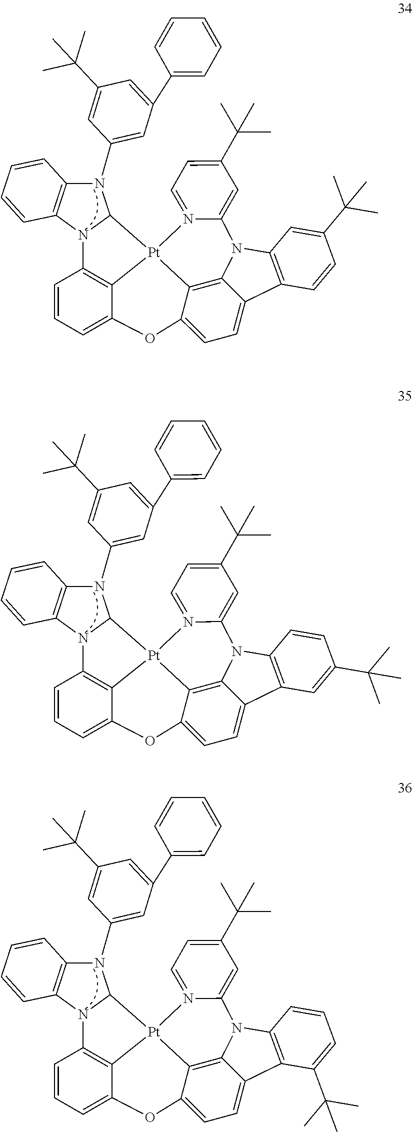

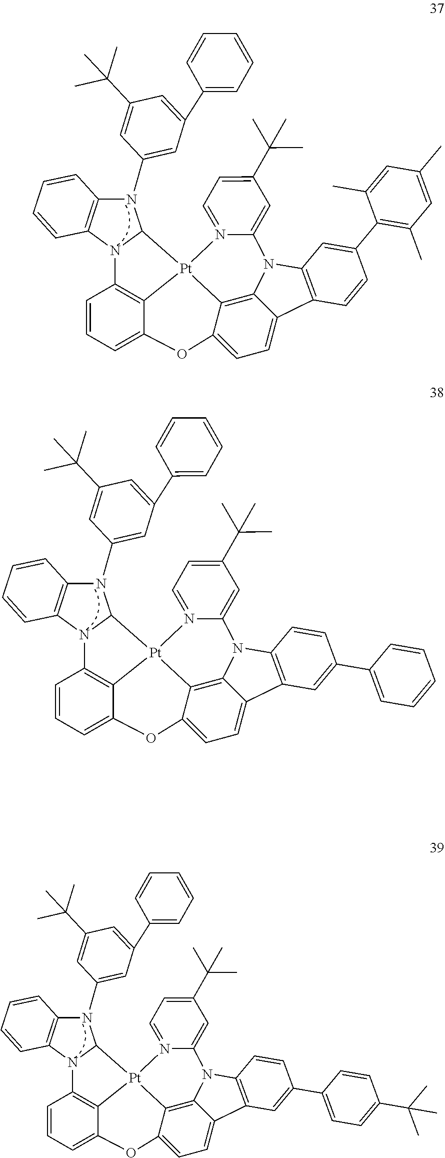

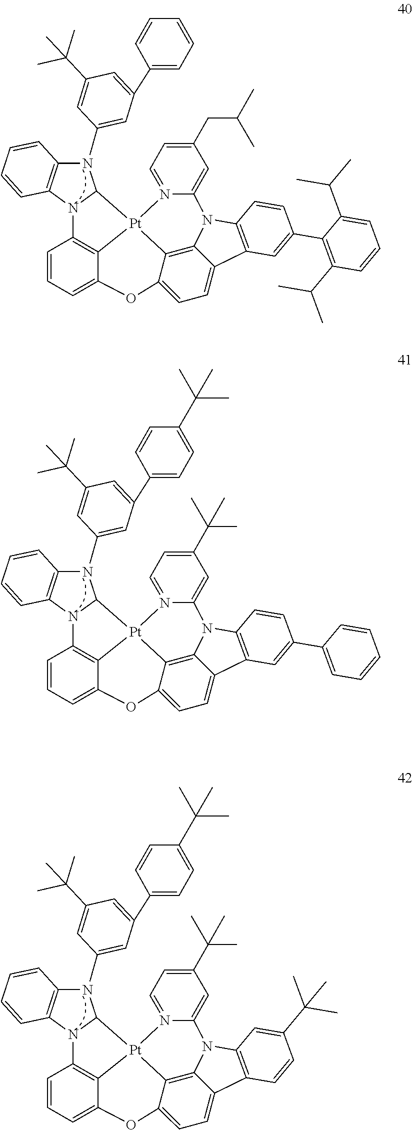

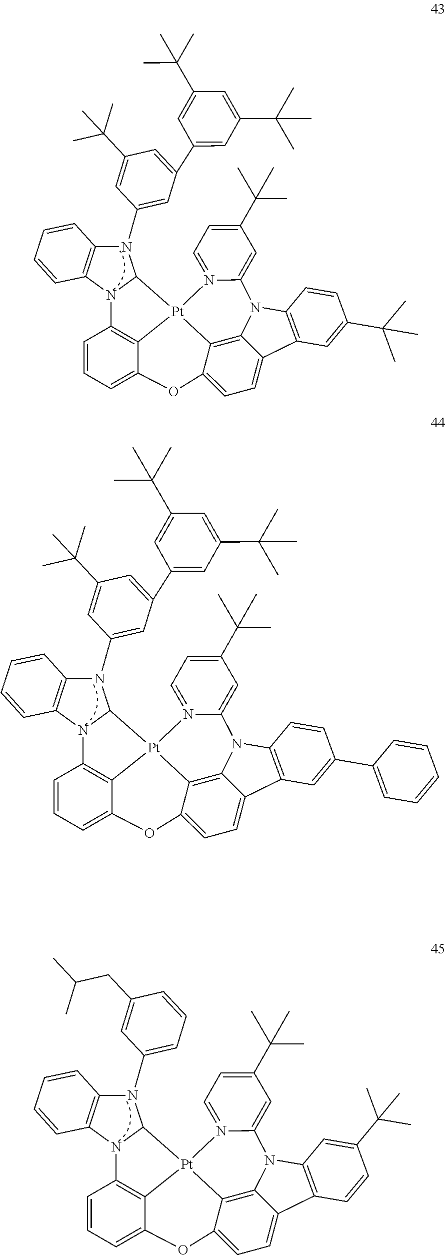

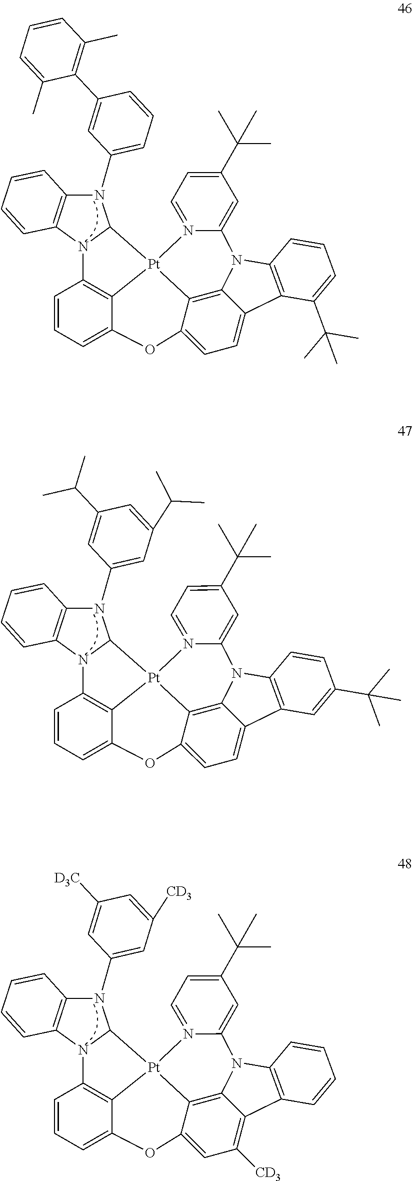

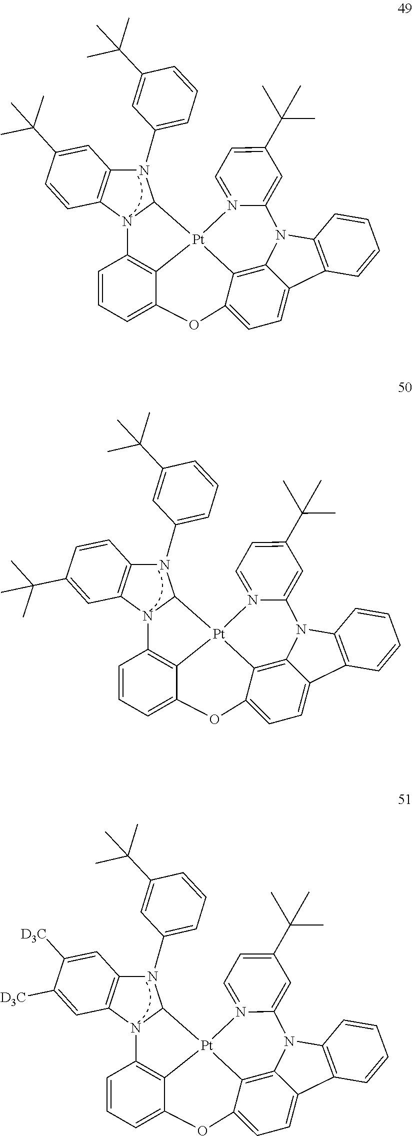

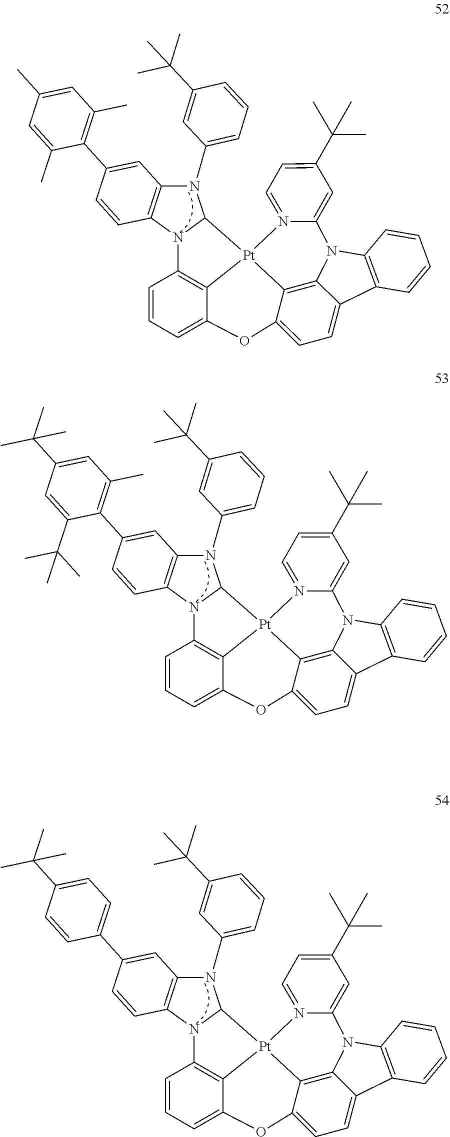

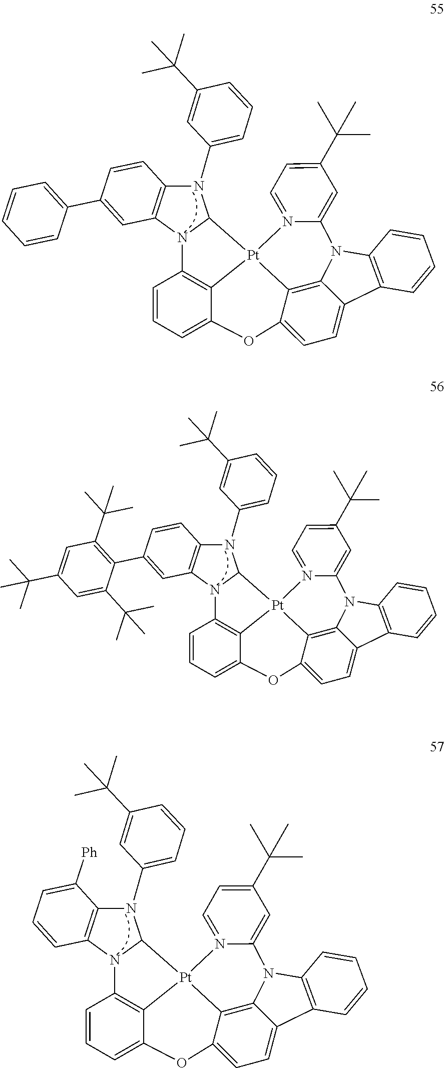

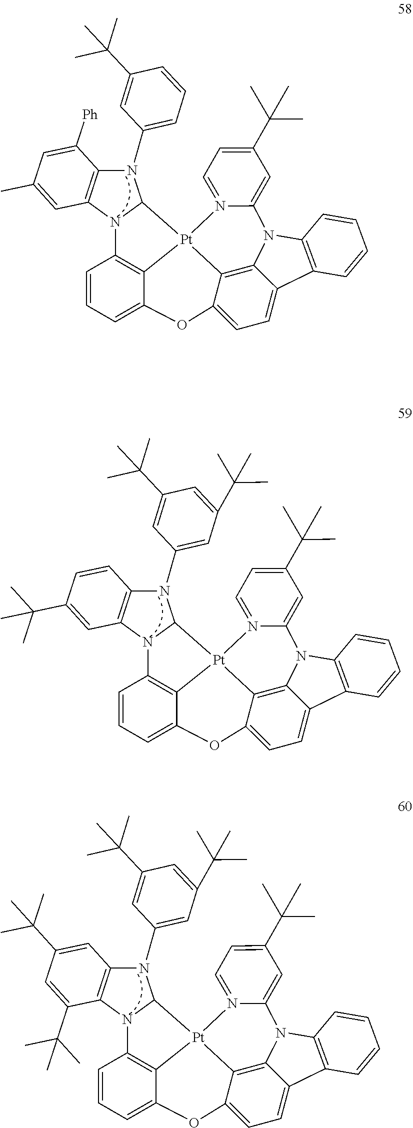

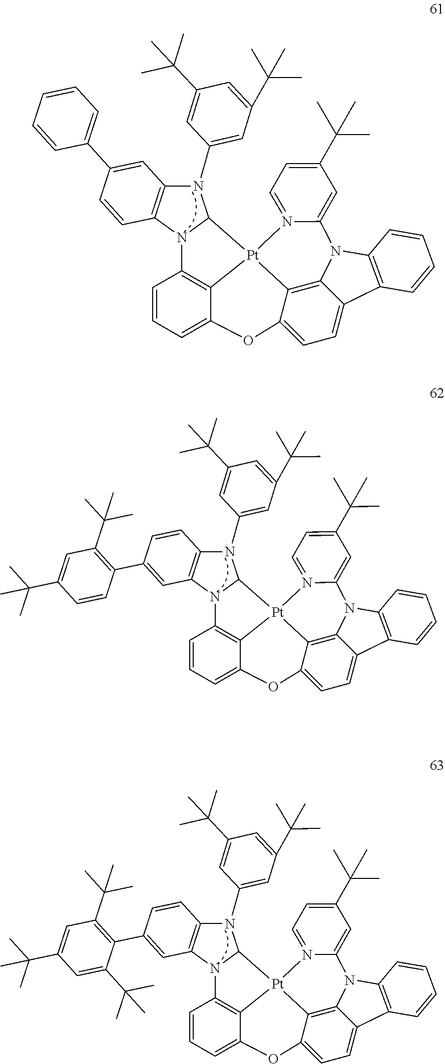

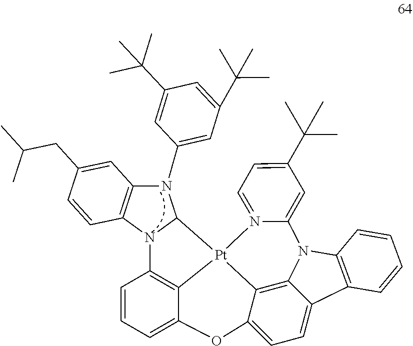

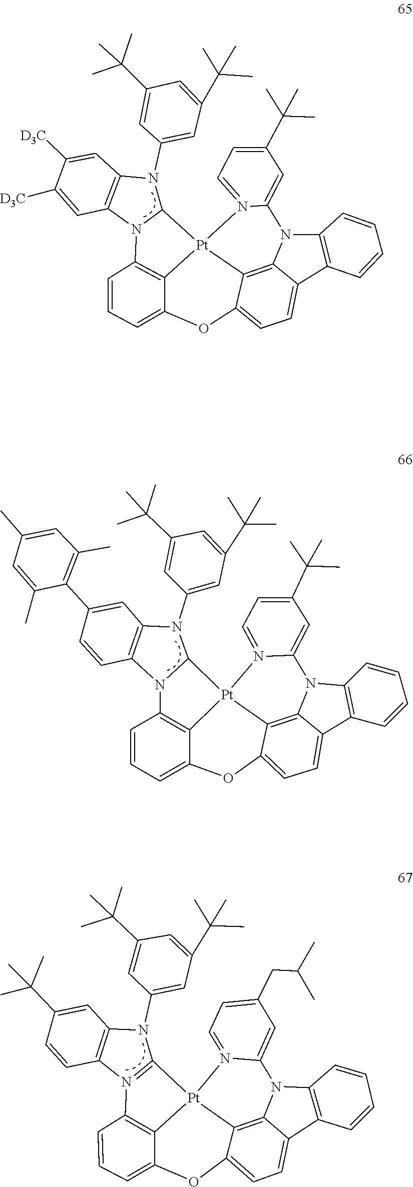

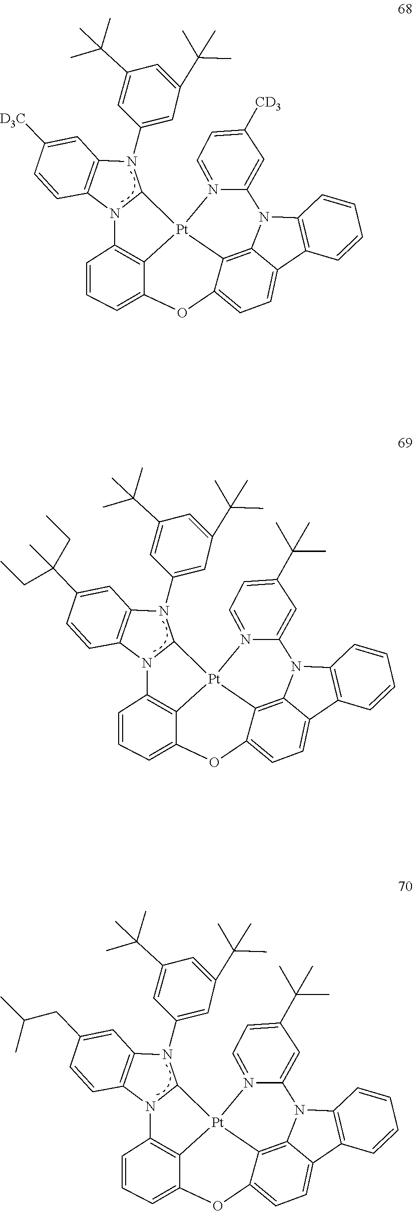

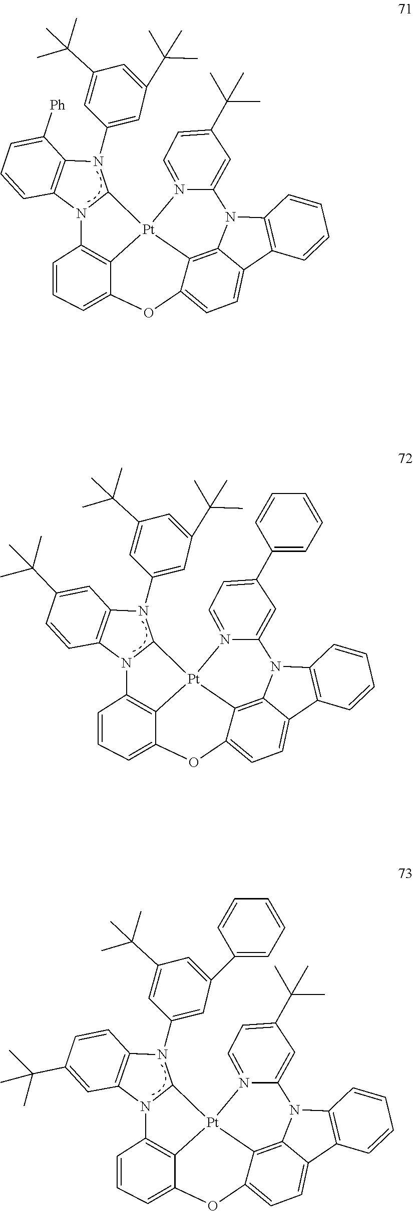

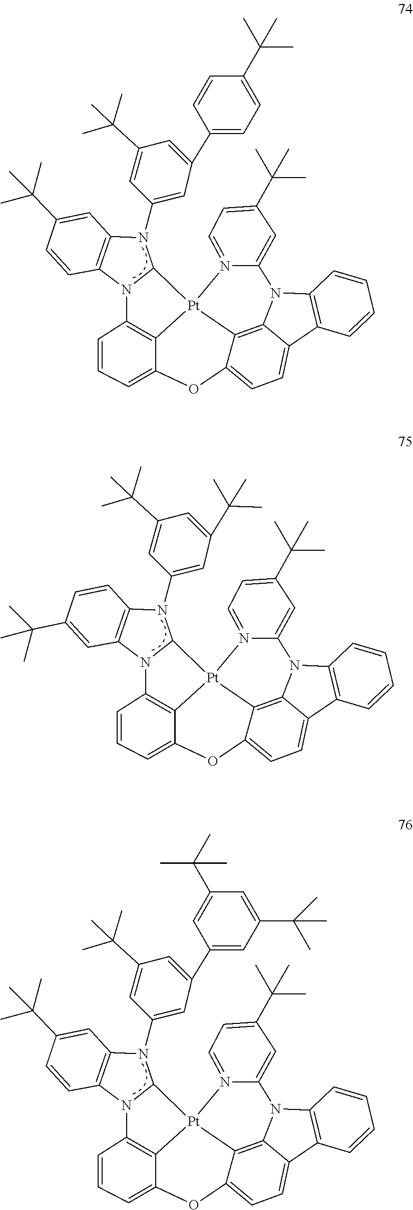

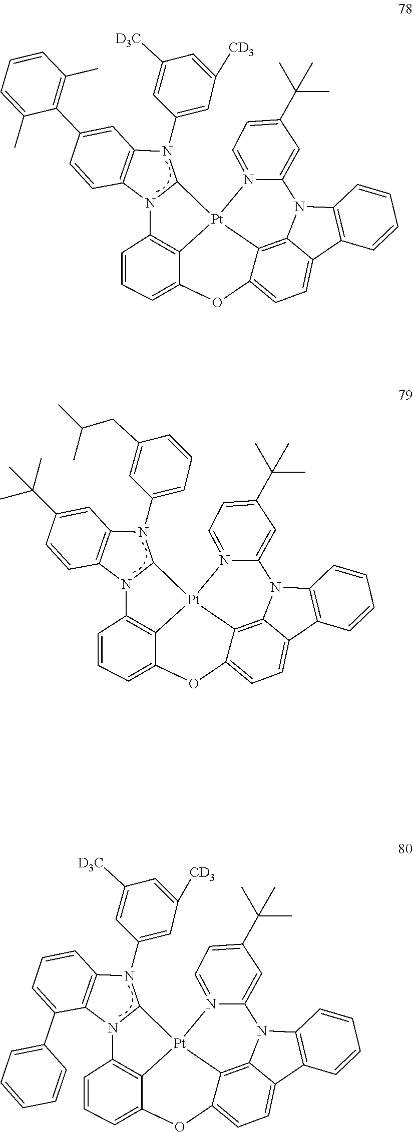

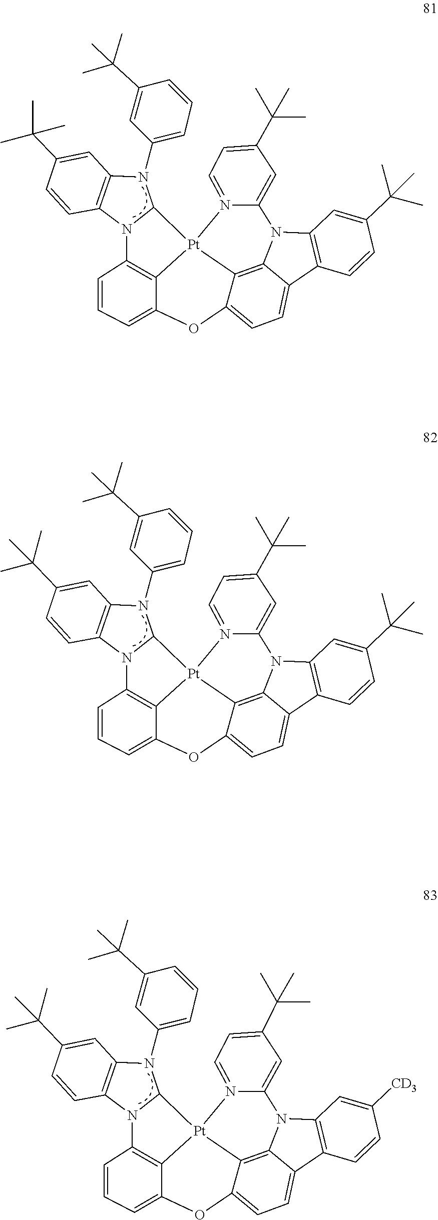

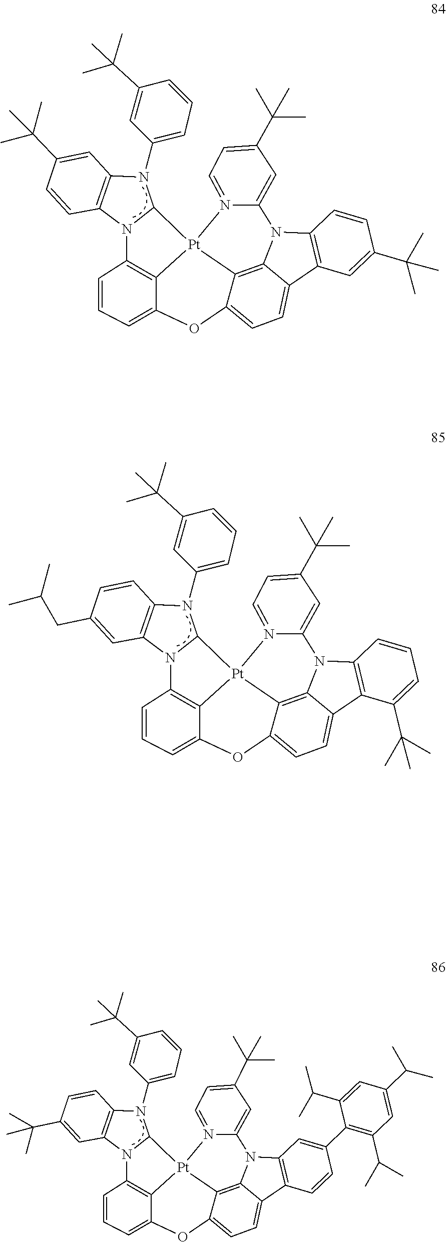

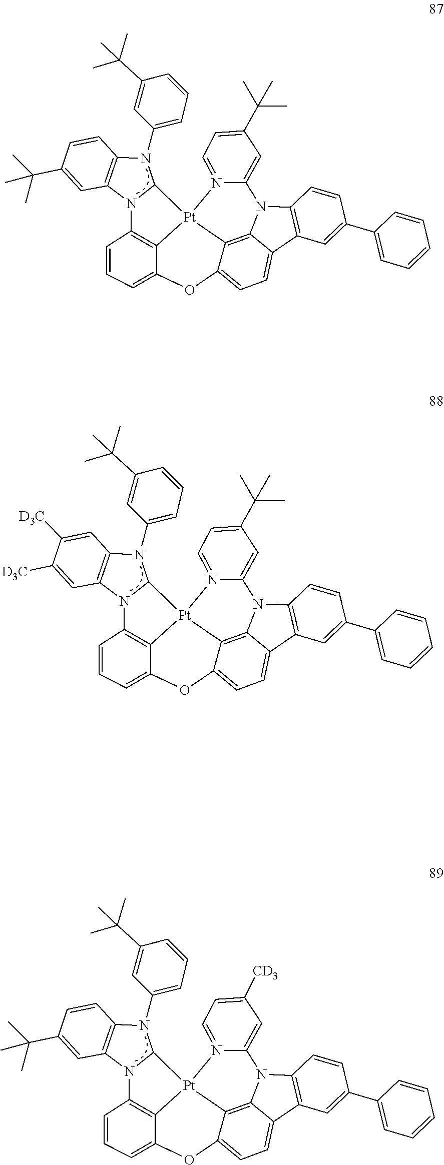

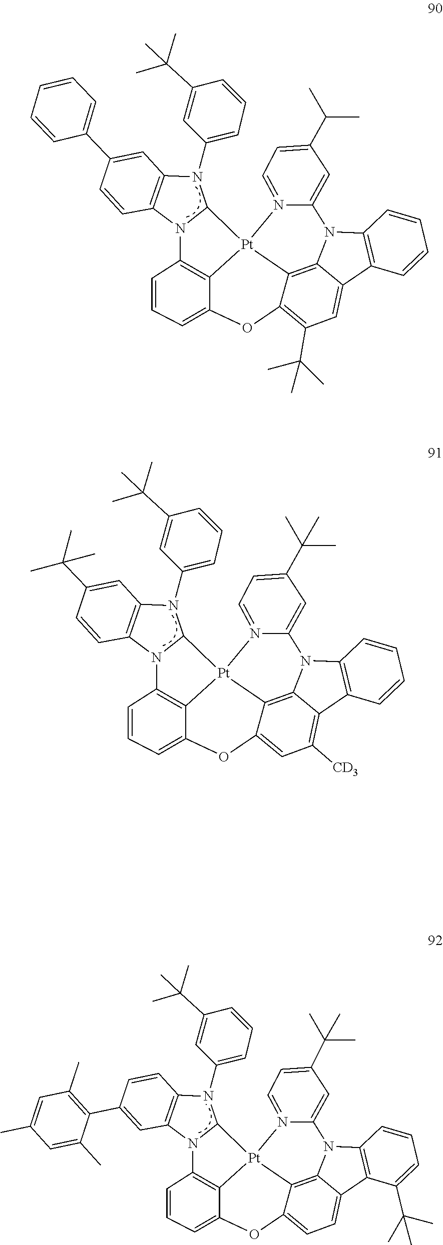

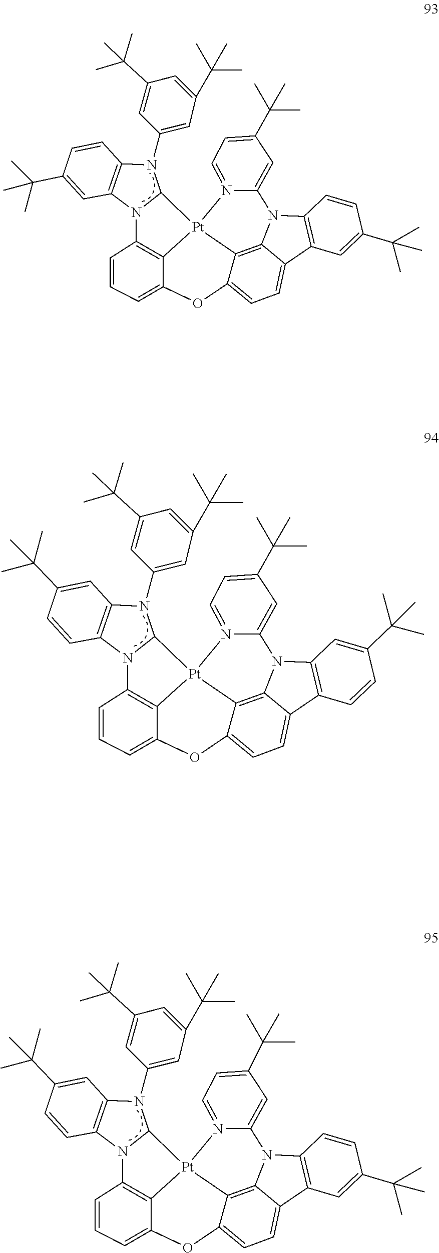

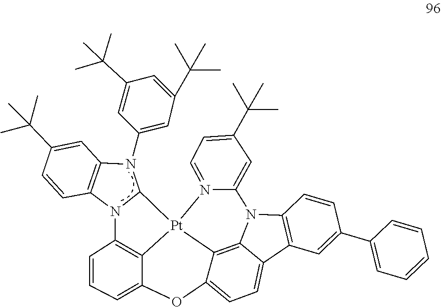

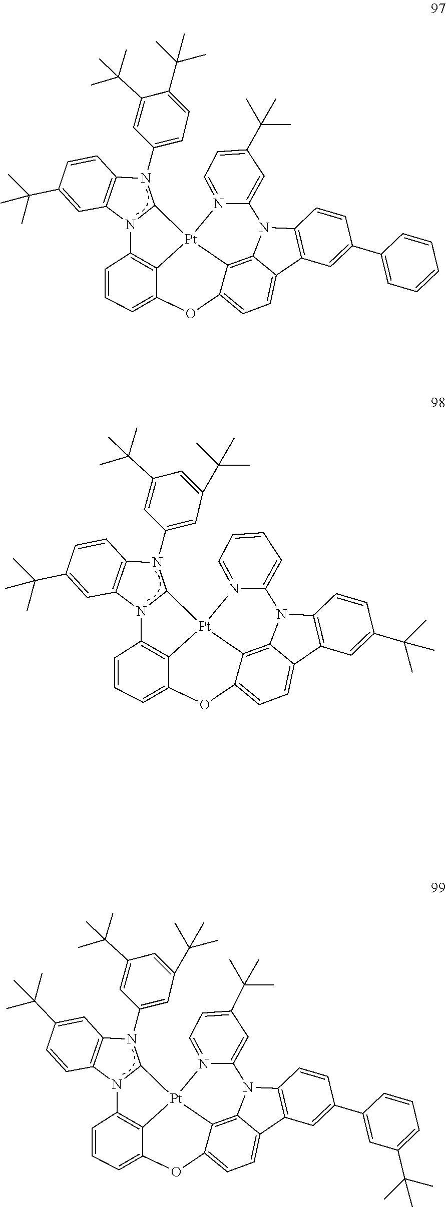

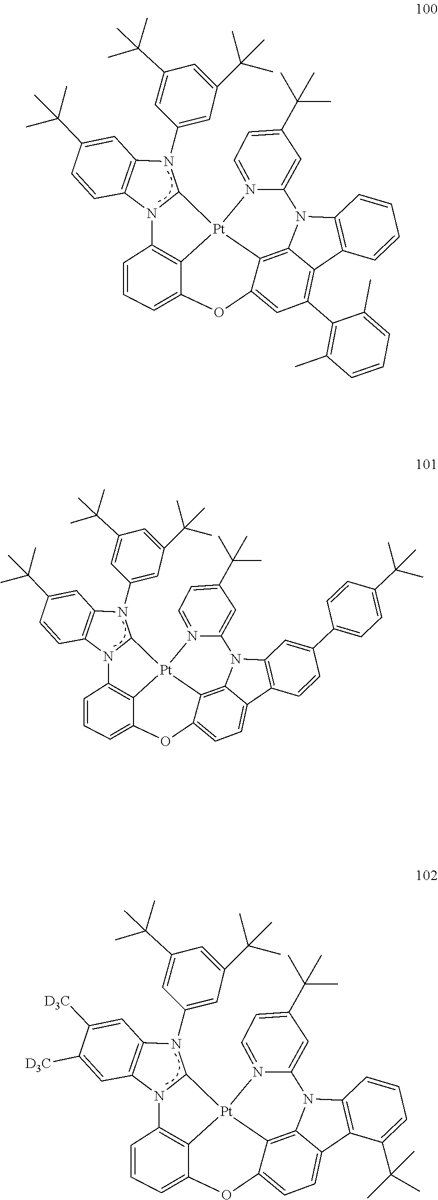

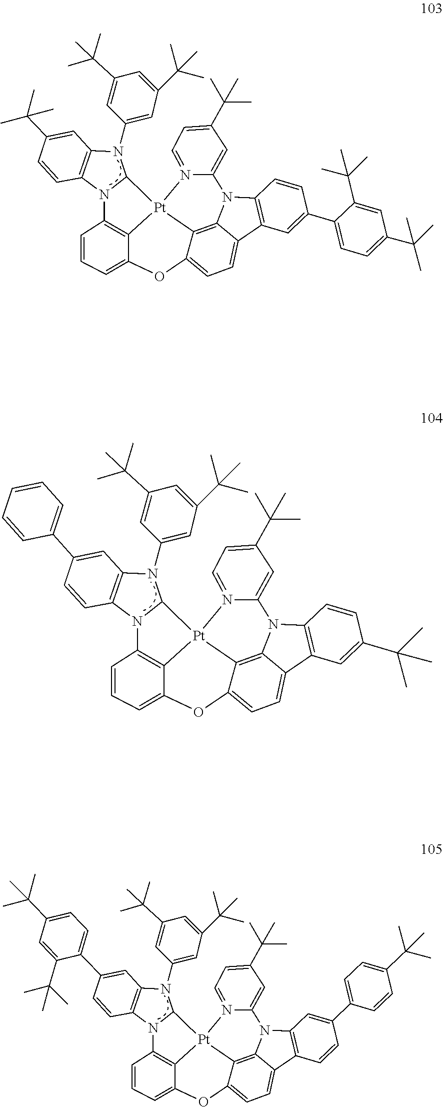

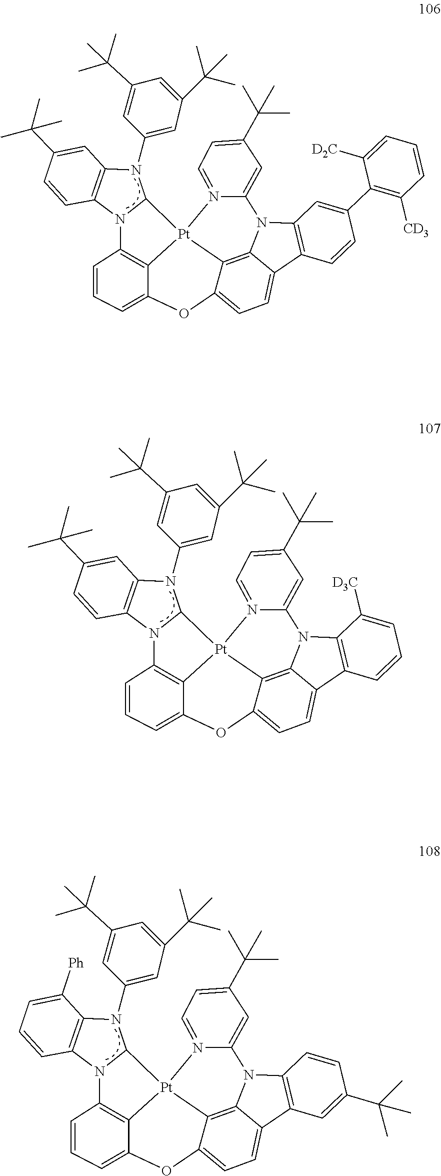

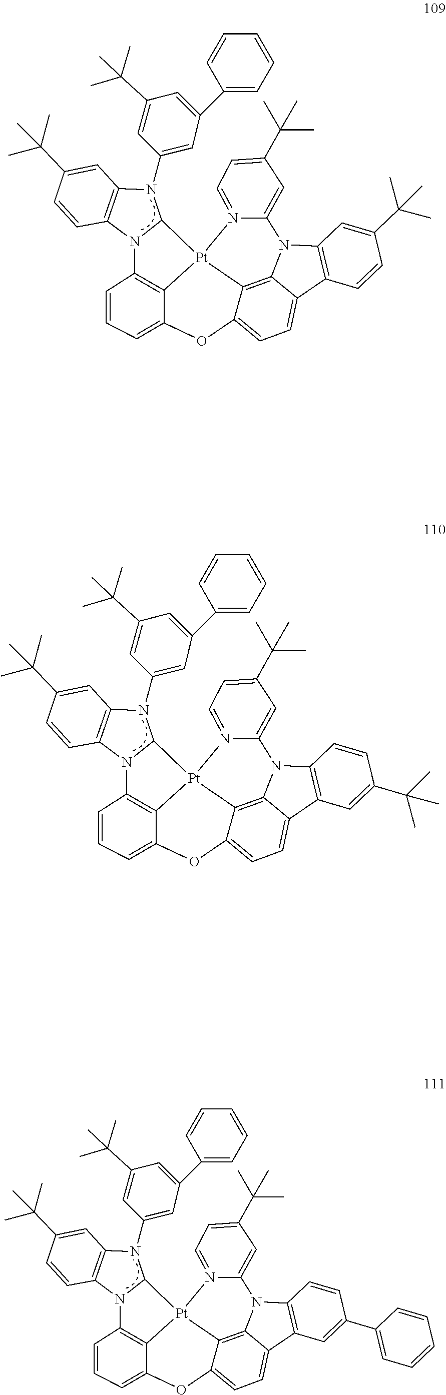

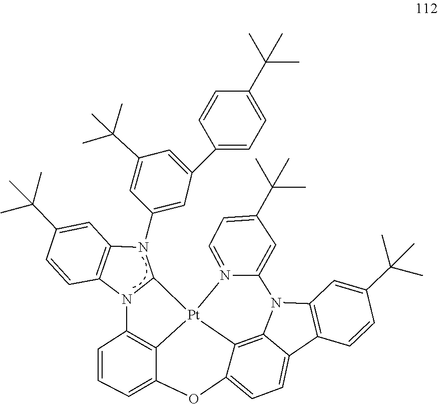

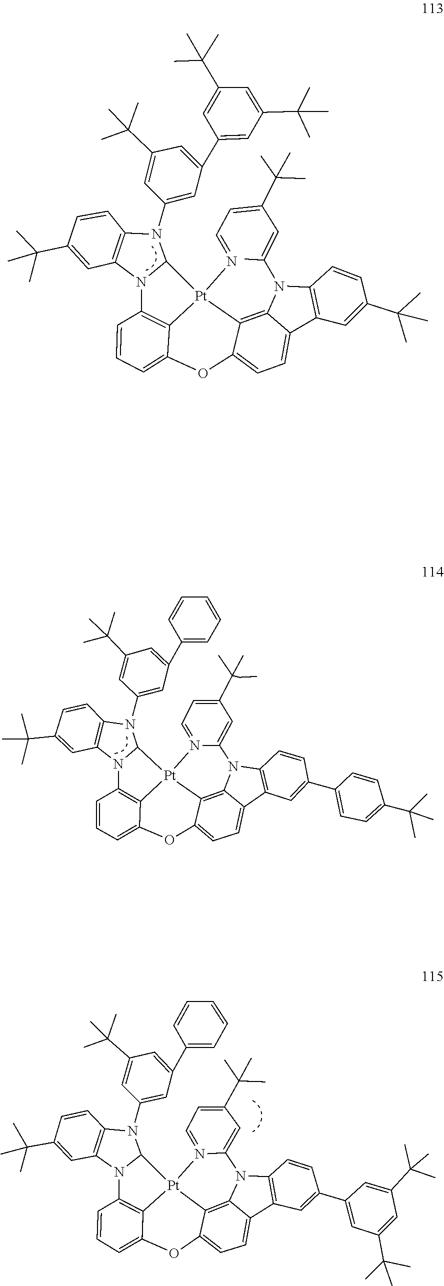

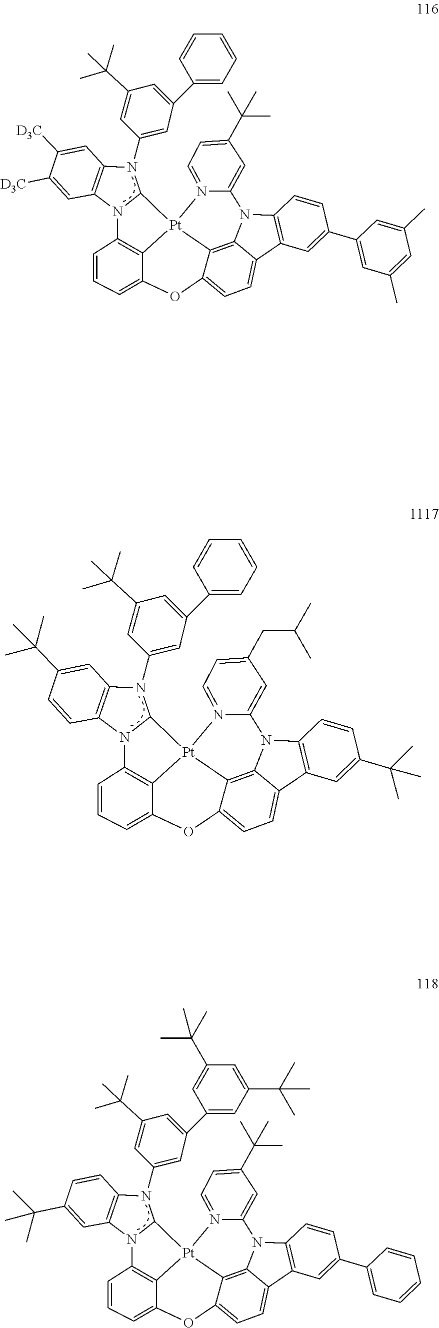

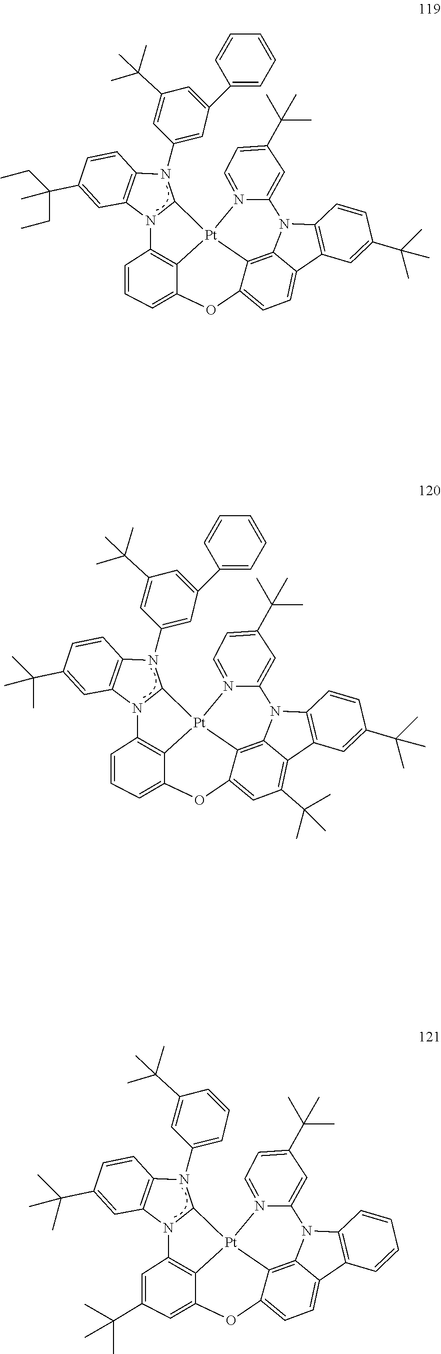

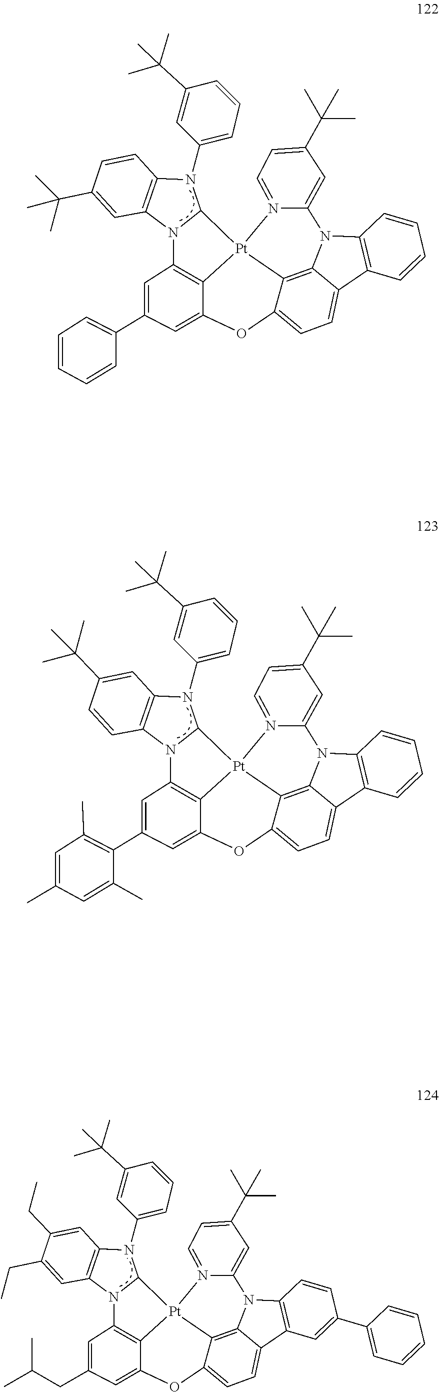

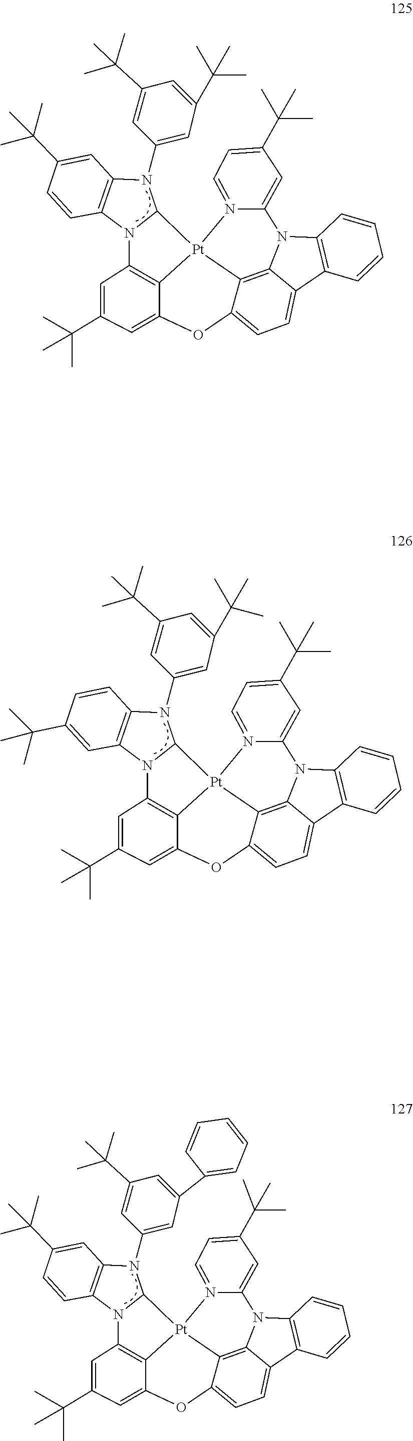

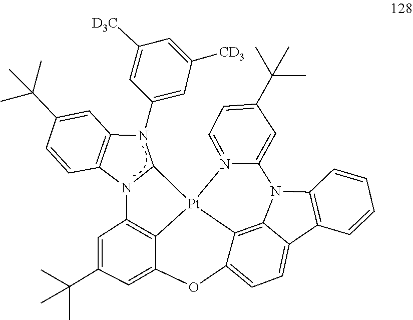

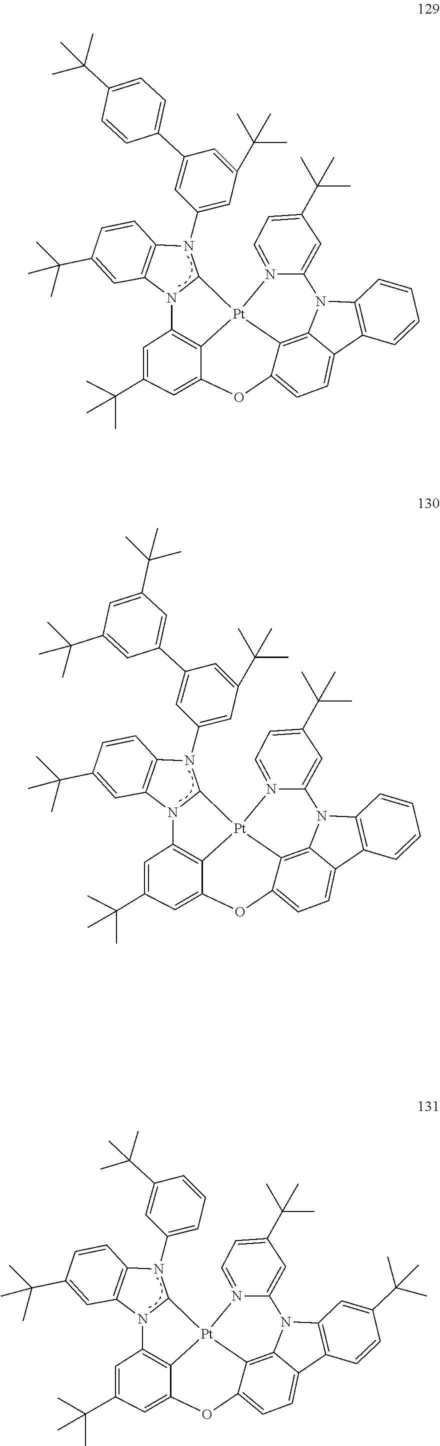

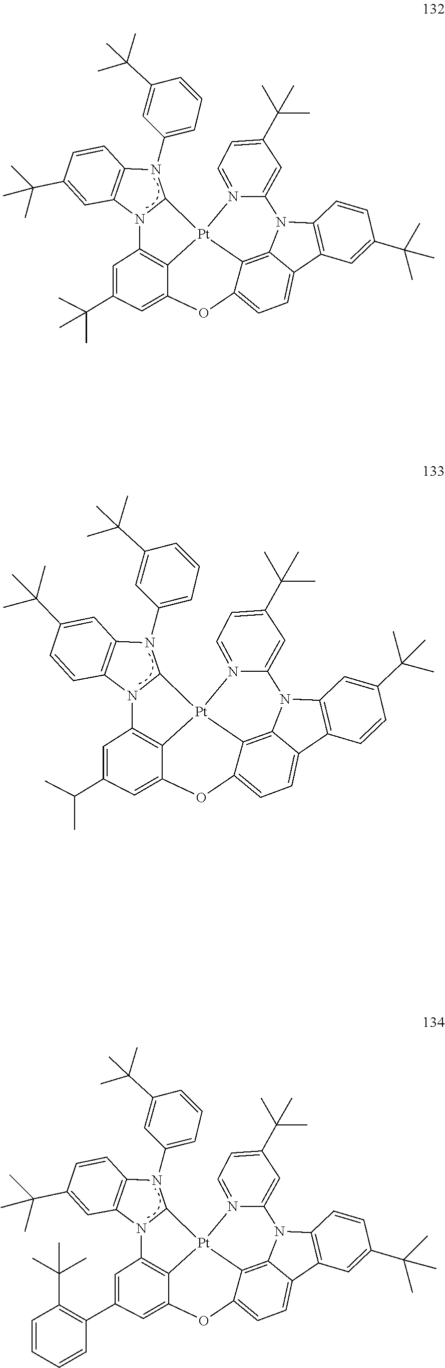

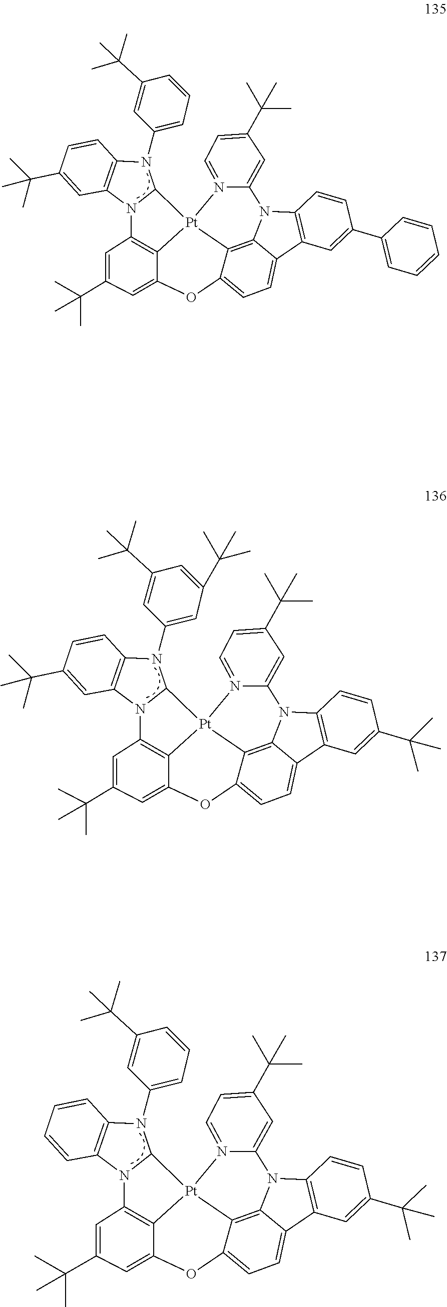

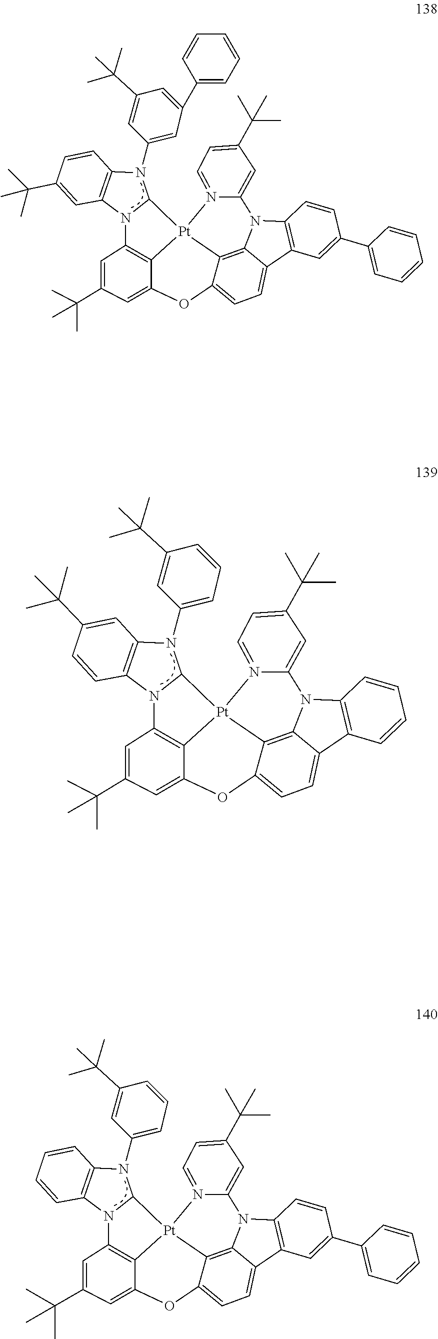

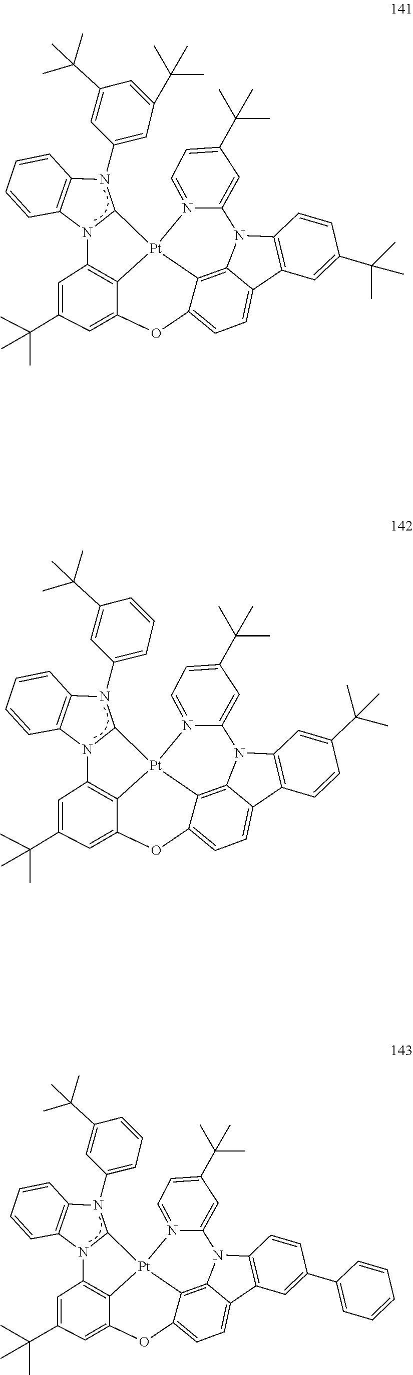

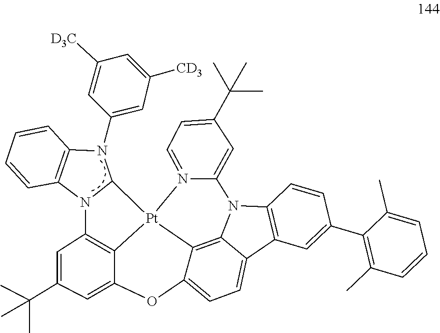

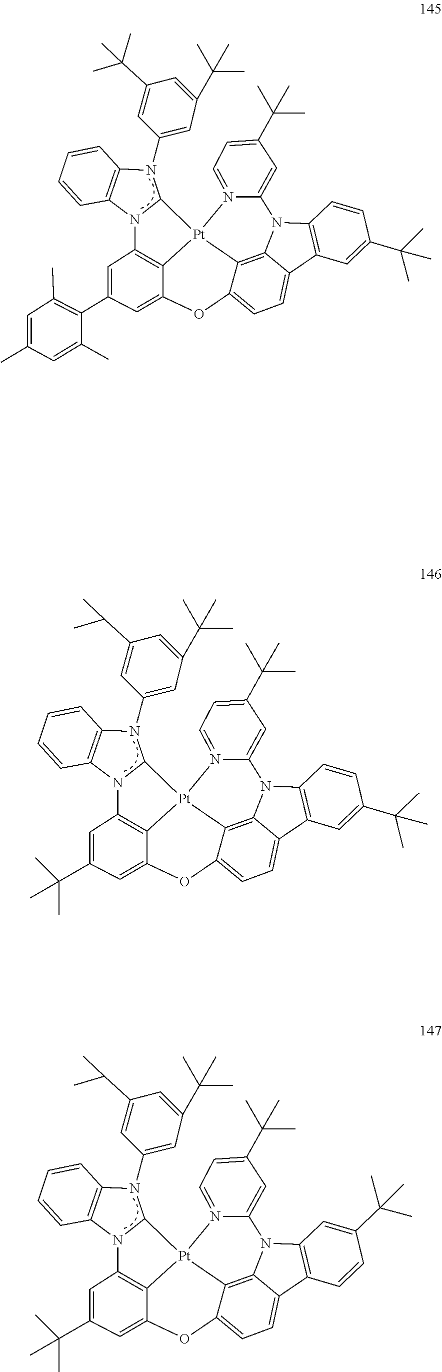

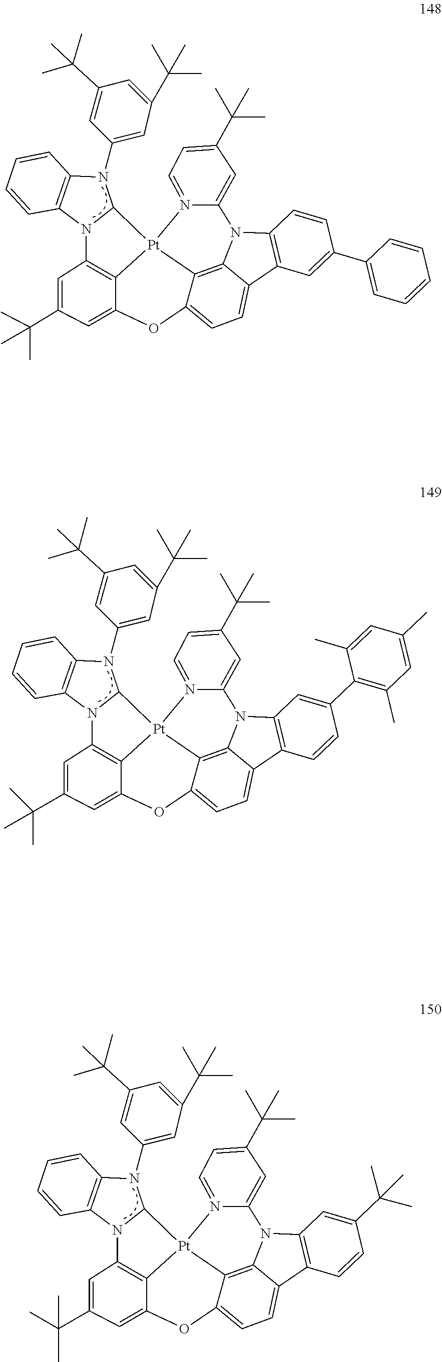

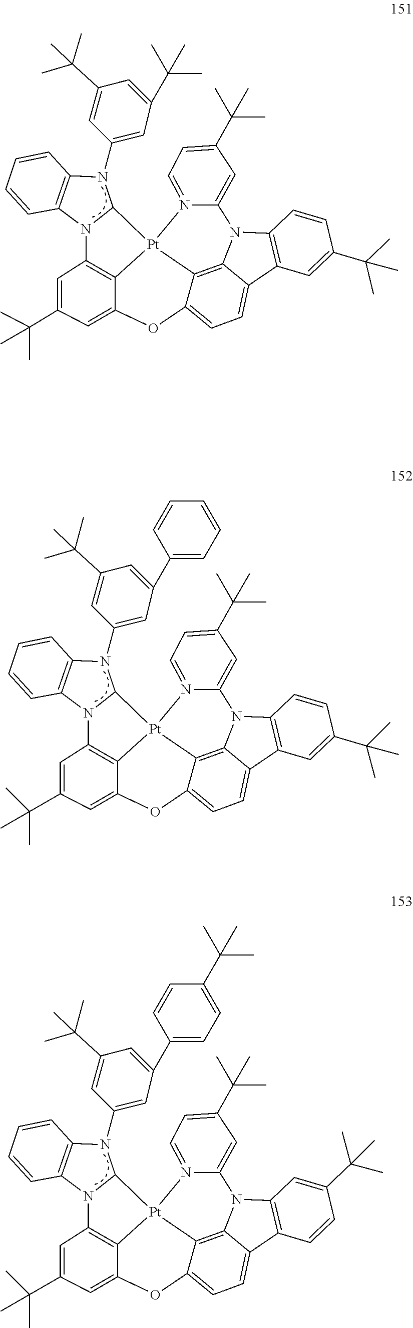

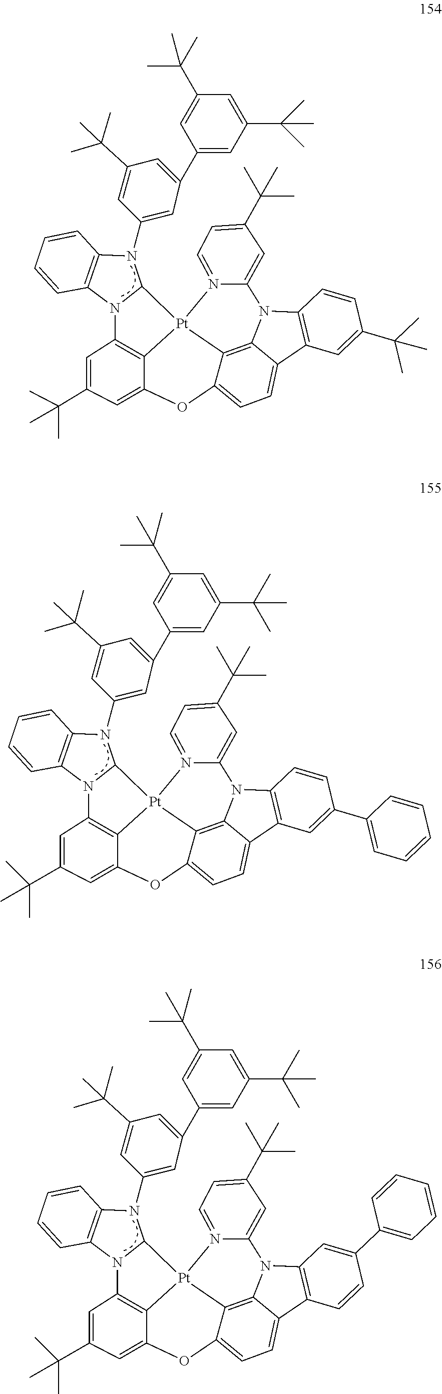

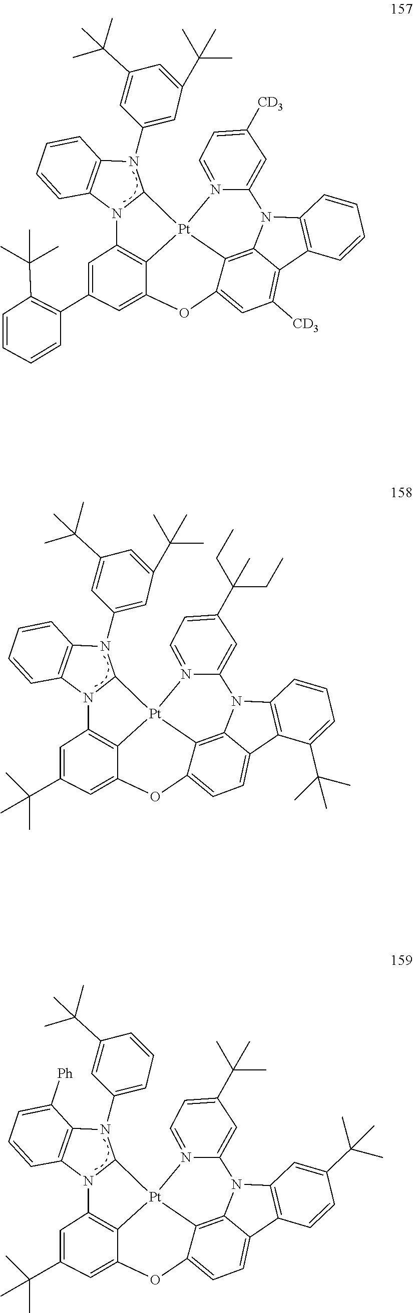

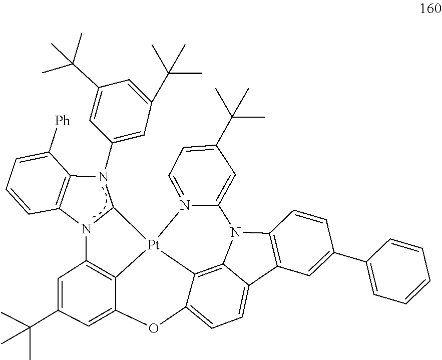

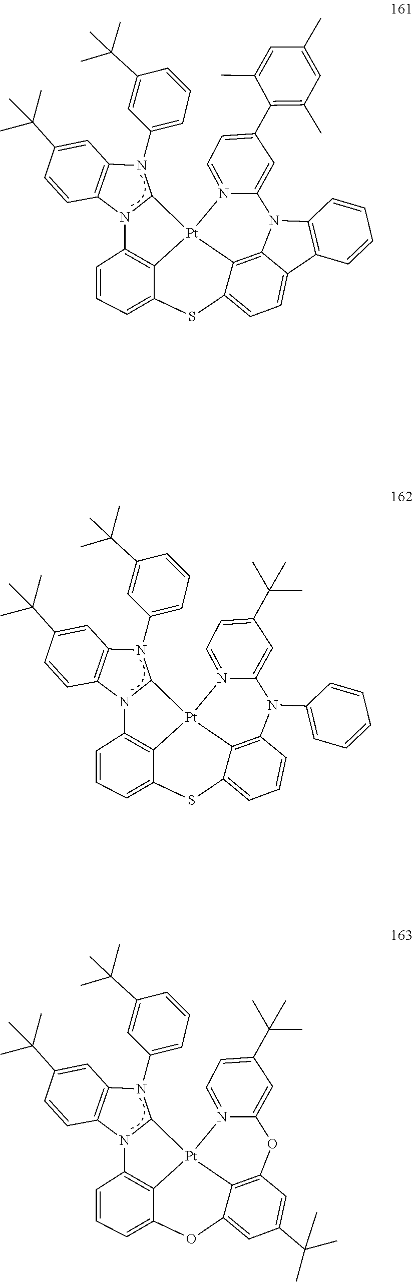

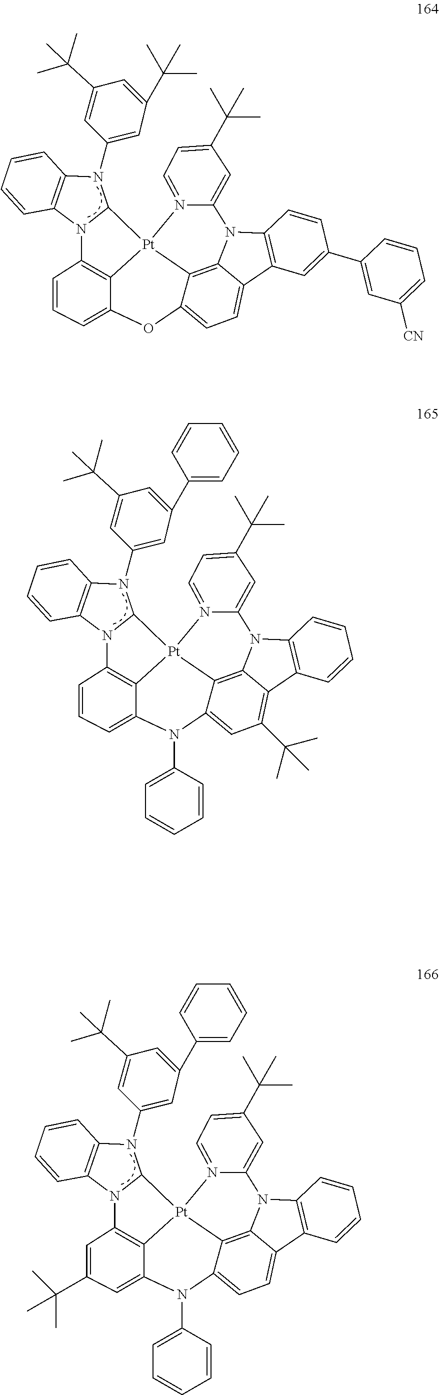

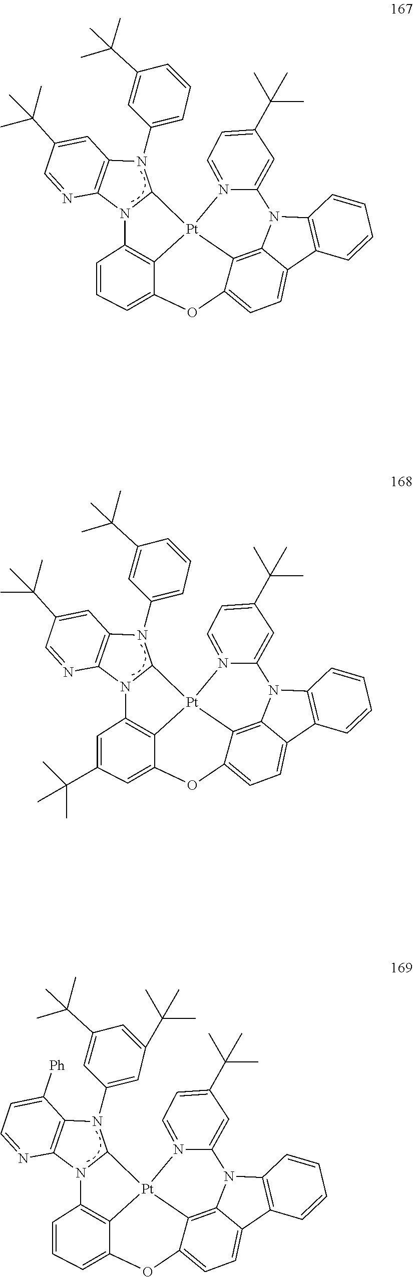

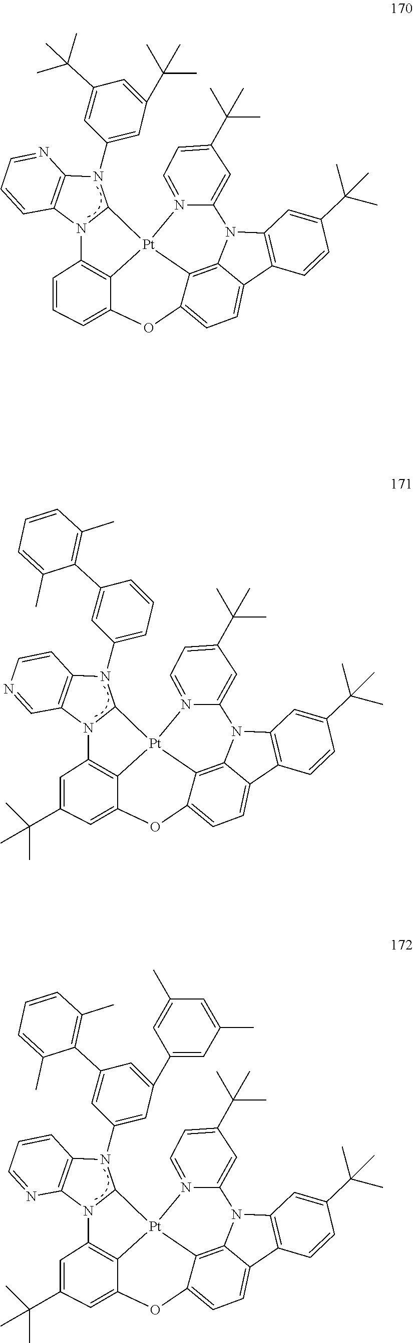

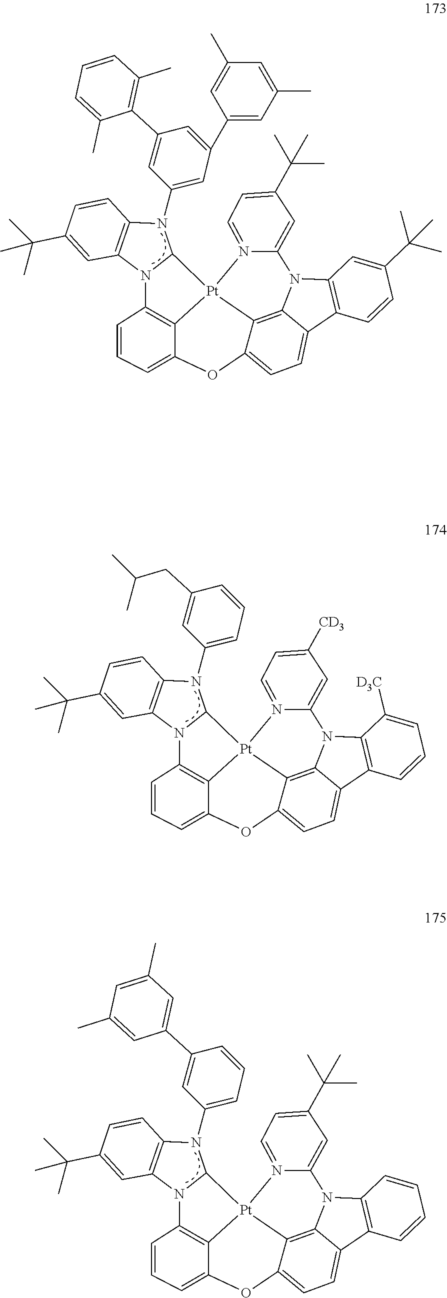

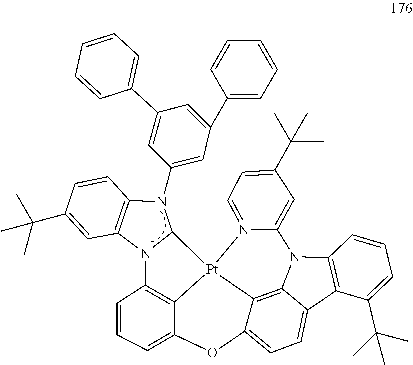

In one or more embodiments, the sensitizer may be represented by Formula 101 or 102, and in this case, the sensitizer may be called a delayed fluorescence sensitizer:

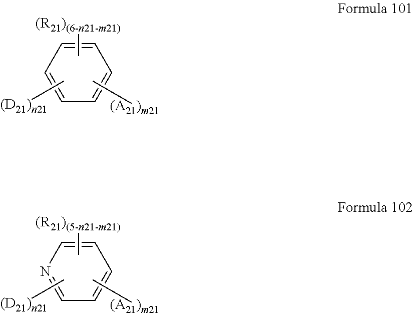

wherein, in Formulae 101 and 102,

A₂₁ is an acceptor group,

D₂₁ is a donor group,

m21 may be 1, 2, or 3, and n21 may be 1, 2, or 3,

the sum of n21 and m21 in Formula 101 may be 6 or less, and the sum of n21 and m21 in Formula 102 may be 5 or less,

R₂₁ may be hydrogen, deuterium, —F, —Cl, —Br, —I, SF₅, a hydroxyl group, a cyano group, a nitro group, an amidino group, a hydrazine group, a hydrazone group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroarylalkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), and a plurality of R₂₁(s) may optionally be bonded to form a substituted or unsubstituted C₅-C₃₀ carbocyclic group or a substituted or unsubstituted C₁-C₃₀ heterocyclic group, and

Q₁ to Q₃ may each independently be: hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a C₁-C₆₀ heteroaryloxy group, a C₁-C₆₀ heteroarylthio group, a monovalent aromatic condensed polycyclic group, a monovalent aromatic heterocondensed polycyclic group, a monovalent non-aromatic condensed polycyclic group, a monovalent non-aromatic condensed heteropolycyclic group; a C₁-C₆₀ alkyl group which is substituted with at least one deuterium, —F, cyano group, a C₁-C₆₀ alkyl group, a C₆-C₆₀ aryl group, or any combination thereof; or a C₆-C₆₀ aryl group which is substituted with at least one deuterium, —F, cyano group, a C₁-C₆₀ alkyl group, a C₆-C₆₀ aryl group, or any combination thereof.

For example, A₂₁ in Formula 101 and 102 may be a substituted or unsubstituted π electron-deficient nitrogen-free cyclic group.

In an embodiment, the π electron-deficient nitrogen-free cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a corogen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group; or a condensed cyclic group of two or more π electron-deficient nitrogen-free cyclic groups, but embodiments of the present disclosure are not limited thereto.

For example, D₂₁ in Formulae 101 and 102 may be: —F, a cyano group, an π-electron deficient nitrogen-containing cyclic group, or any combination thereof;

a C₁-C₆₀ alkyl group, an π-electron deficient nitrogen-containing cyclic group, or an π electron-deficient nitrogen-free cyclic group, each substituted with at least one —F a cyano group, or any combination thereof; or a π-electron deficient nitrogen-containing cyclic group, each substituted with at least one deuterium, a C₁-C₆₀ alkyl group, a π-electron deficient nitrogen-containing cyclic group, a π electron-deficient nitrogen-free cyclic group, or any combination thereof.

In an embodiment, the π electron-deficient nitrogen-free cyclic group is the same as described above.

The term “π electron-deficient nitrogen-containing cyclic group” used herein refers to a cyclic group having at least one *—N═*′ moiety, and, for example, may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, a benzimidazolobenzimidazole group, or a condensed cyclic group of two or more π electron-deficient nitrogen-containing cyclic groups.

In one or more embodiments, the sensitizer may be a compound of Groups VII to XI, but embodiments of the present disclosure are not limited thereto:

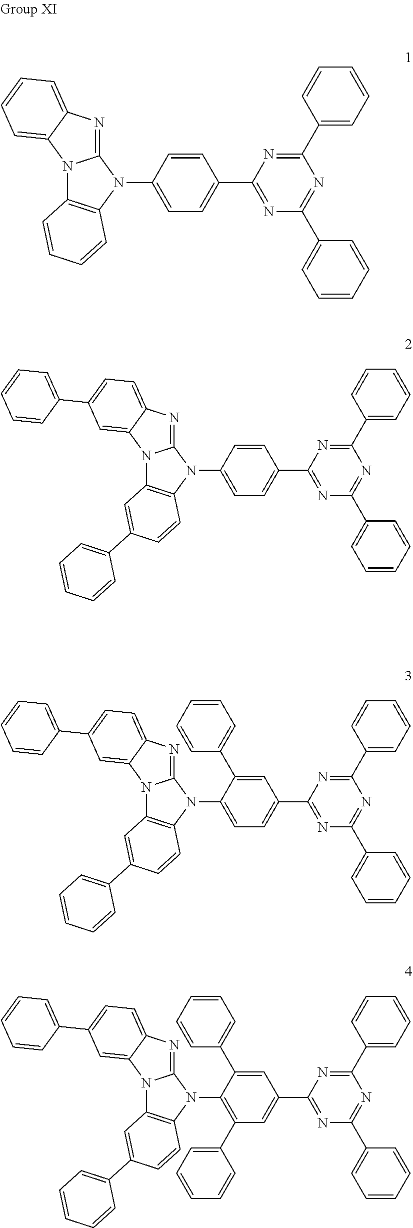 [SMILES: c1ccc(-c2nc(-c3ccccc3)nc(-c3ccc(-n4c5ccccc5n5c6ccccc6nc45)cc3)n2)cc1 ; c1ccc(-c2ccc3nc4n(-c5ccc(-c6nc(-c7ccccc7)nc(-c7ccccc7)n6)cc5)c5ccc(-c6ccccc6)cc5n4c3c2)cc1 ; c1ccc(-c2ccc3nc4n(-c5ccc(-c6nc(-c7ccccc7)nc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6nc(-c7ccccc7)nc(-c7ccccc7)n6)cc5-c5ccccc5)c5ccc(-c6ccccc6)cc5n4c3c2)cc1]

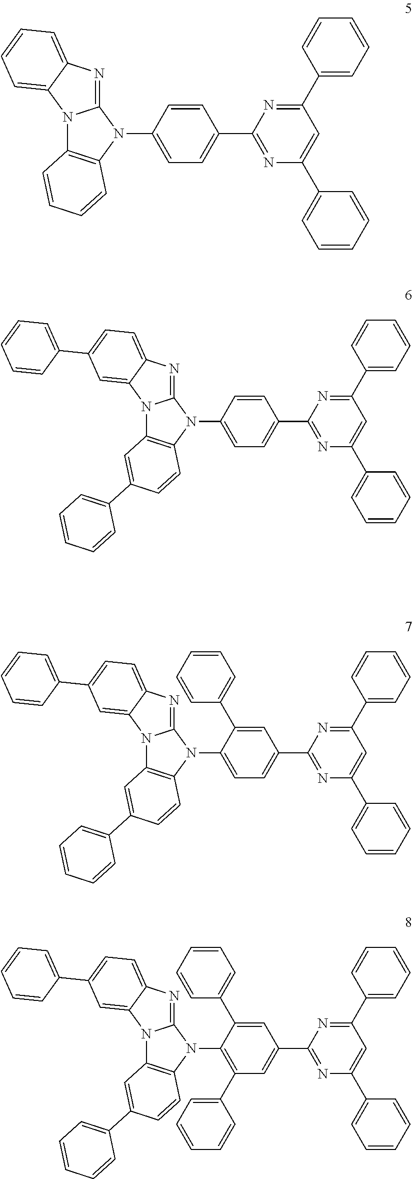 [SMILES: c1ccc(-c2cc(-c3ccccc3)nc(-c3ccc(-n4c5ccccc5n5c6ccccc6nc45)cc3)n2)cc1 ; c1ccc(-c2ccc3nc4n(-c5ccc(-c6nc(-c7ccccc7)cc(-c7ccccc7)n6)cc5)c5ccc(-c6ccccc6)cc5n4c3c2)cc1 ; c1ccc(-c2ccc3nc4n(-c5ccc(-c6nc(-c7ccccc7)cc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6nc(-c7ccccc7)cc(-c7ccccc7)n6)cc5-c5ccccc5)c5ccc(-c6ccccc6)cc5n4c3c2)cc1]

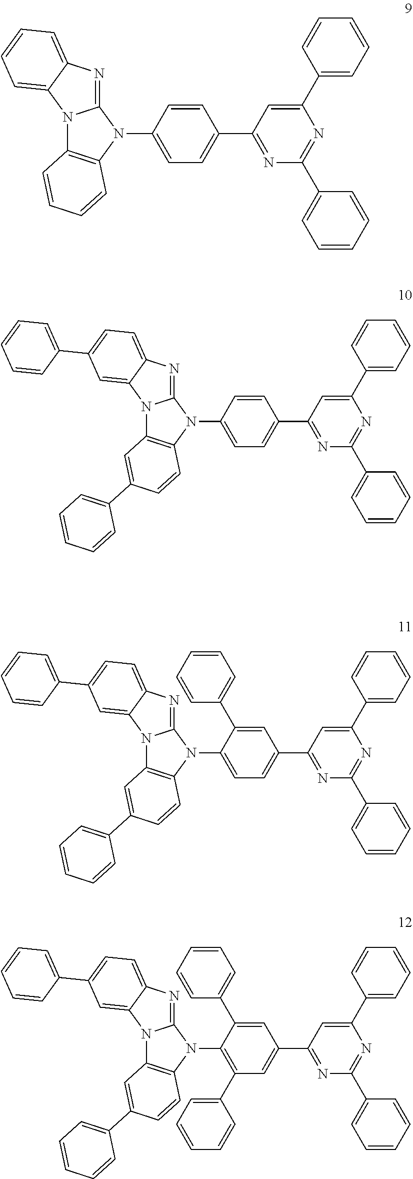 [SMILES: c1ccc(-c2cc(-c3ccc(-n4c5ccccc5n5c6ccccc6nc45)cc3)nc(-c3ccccc3)n2)cc1 ; c1ccc(-c2ccc3nc4n(-c5ccc(-c6cc(-c7ccccc7)nc(-c7ccccc7)n6)cc5)c5ccc(-c6ccccc6)cc5n4c3c2)cc1 ; c1ccc(-c2ccc3nc4n(-c5ccc(-c6cc(-c7ccccc7)nc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6cc(-c7ccccc7)nc(-c7ccccc7)n6)cc5-c5ccccc5)c5ccc(-c6ccccc6)cc5n4c3c2)cc1]

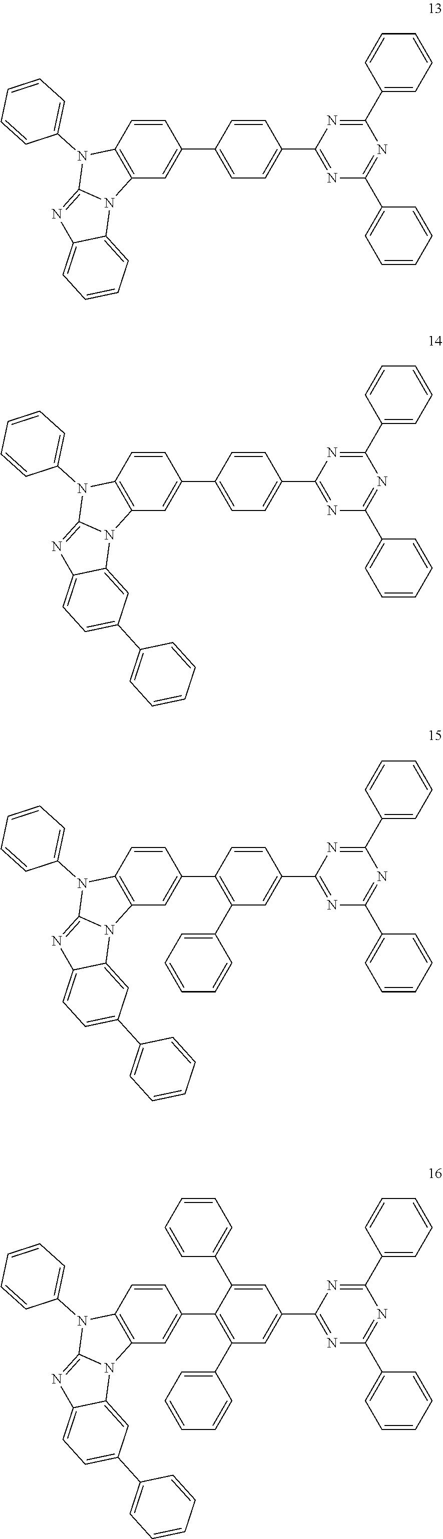 [SMILES: c1ccc(-c2nc(-c3ccccc3)nc(-c3ccc(-c4ccc5c(c4)n4c6ccccc6nc4n5-c4ccccc4)cc3)n2)cc1 ; c1ccc(-c2ccc3nc4n(-c5ccccc5)c5ccc(-c6ccc(-c7nc(-c8ccccc8)nc(-c8ccccc8)n7)cc6)cc5n4c3c2)cc1 ; c1ccc(-c2ccc3nc4n(-c5ccccc5)c5ccc(-c6ccc(-c7nc(-c8ccccc8)nc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7nc(-c8ccccc8)nc(-c8ccccc8)n7)cc6-c6ccccc6)cc5n4c3c2)cc1]

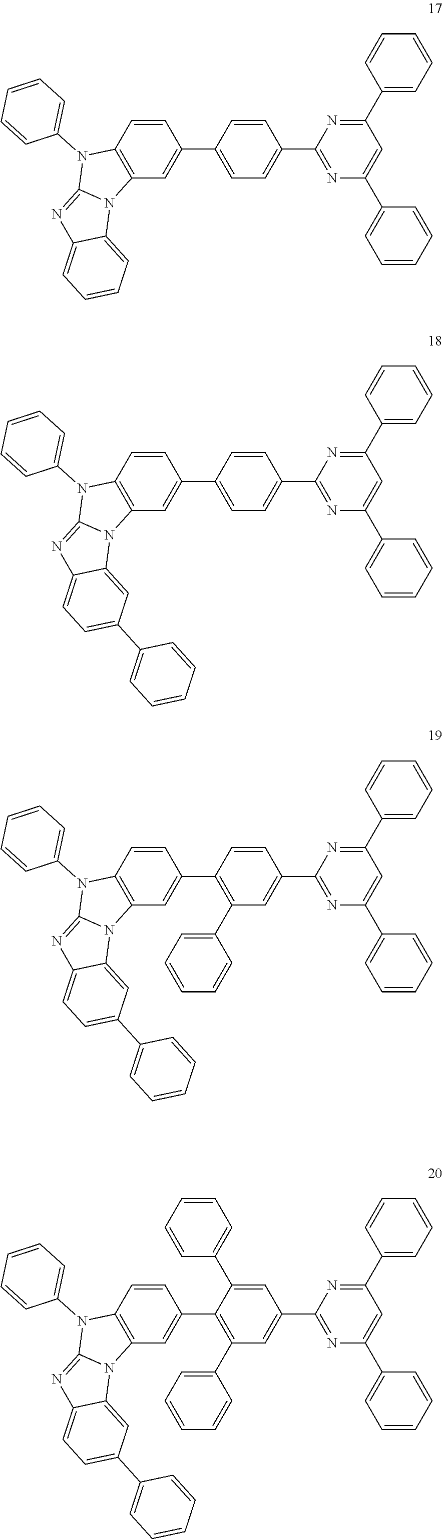 [SMILES: c1ccc(-c2cc(-c3ccccc3)nc(-c3ccc(-c4ccc5c(c4)n4c6ccccc6nc4n5-c4ccccc4)cc3)n2)cc1 ; c1ccc(-c2ccc3nc4n(-c5ccccc5)c5ccc(-c6ccc(-c7nc(-c8ccccc8)cc(-c8ccccc8)n7)cc6)cc5n4c3c2)cc1 ; c1ccc(-c2ccc3nc4n(-c5ccccc5)c5ccc(-c6ccc(-c7nc(-c8ccccc8)cc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7nc(-c8ccccc8)cc(-c8ccccc8)n7)cc6-c6ccccc6)cc5n4c3c2)cc1]

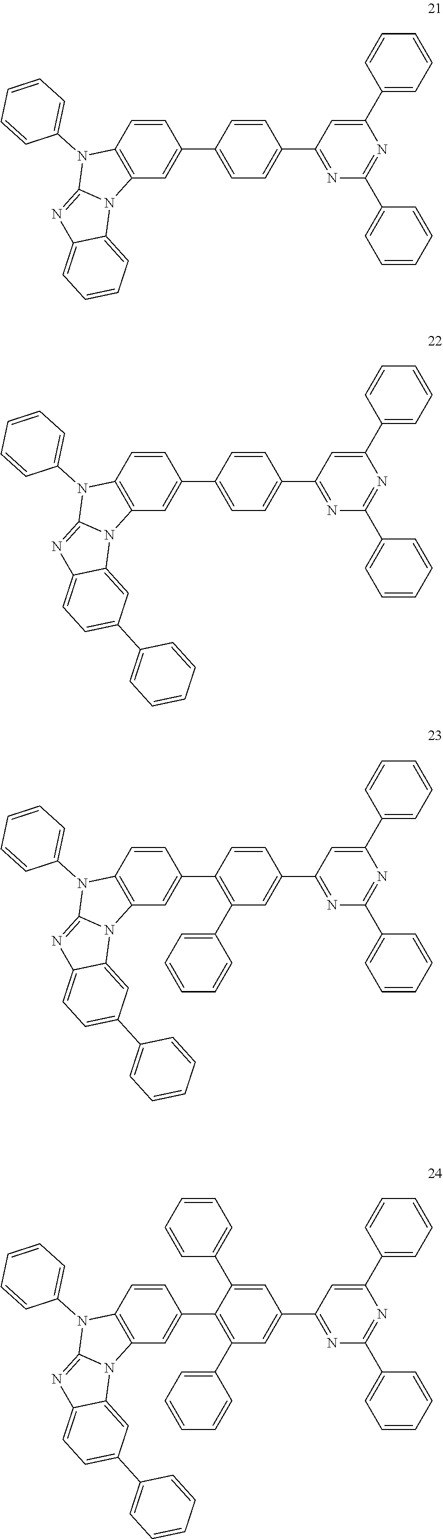 [SMILES: c1ccc(-c2cc(-c3ccc(-c4ccc5c(c4)n4c6ccccc6nc4n5-c4ccccc4)cc3)nc(-c3ccccc3)n2)cc1 ; c1ccc(-c2ccc3nc4n(-c5ccccc5)c5ccc(-c6ccc(-c7cc(-c8ccccc8)nc(-c8ccccc8)n7)cc6)cc5n4c3c2)cc1 ; c1ccc(-c2ccc3nc4n(-c5ccccc5)c5ccc(-c6ccc(-c7cc(-c8ccccc8)nc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7cc(-c8ccccc8)nc(-c8ccccc8)n7)cc6-c6ccccc6)cc5n4c3c2)cc1]

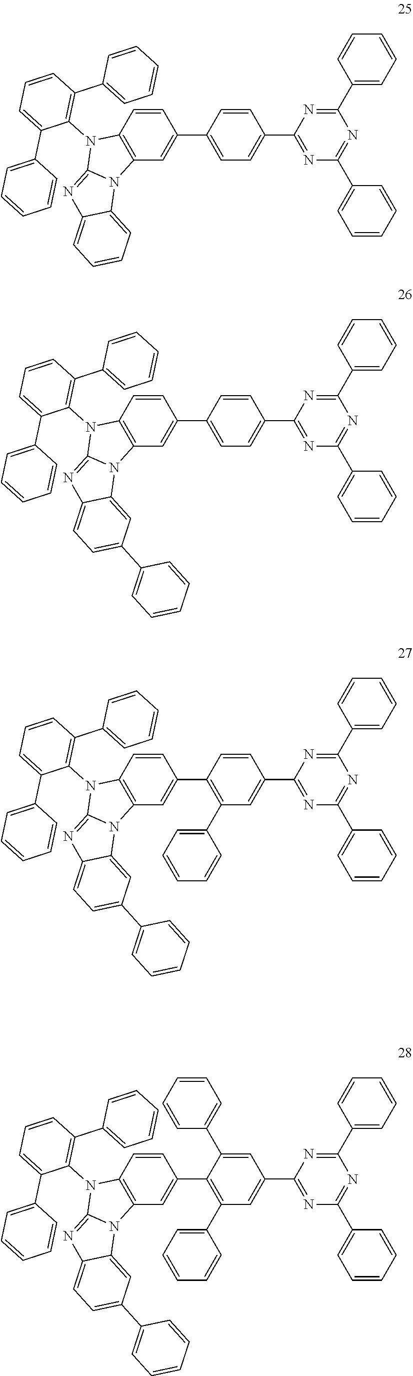 [SMILES: c1ccc(-c2nc(-c3ccccc3)nc(-c3ccc(-c4ccc5c(c4)n4c6ccccc6nc4n5-c4c(-c5ccccc5)cccc4-c4ccccc4)cc3)n2)cc1 ; c1ccc(-c2ccc3nc4n(-c5c(-c6ccccc6)cccc5-c5ccccc5)c5ccc(-c6ccc(-c7nc(-c8ccccc8)nc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7nc(-c8ccccc8)nc(-c8ccccc8)n7)cc6-c6ccccc6)cc5n4c3c2)cc1 ; c1ccc(-c2ccc3nc4n(-c5c(-c6ccccc6)cccc5-c5ccccc5)c5ccc(-c6c(-c7ccccc7)cc(-c7nc(-c8ccccc8)nc(-c8ccccc8)n7)cc6-c6ccccc6)cc5n4c3c2)cc1]

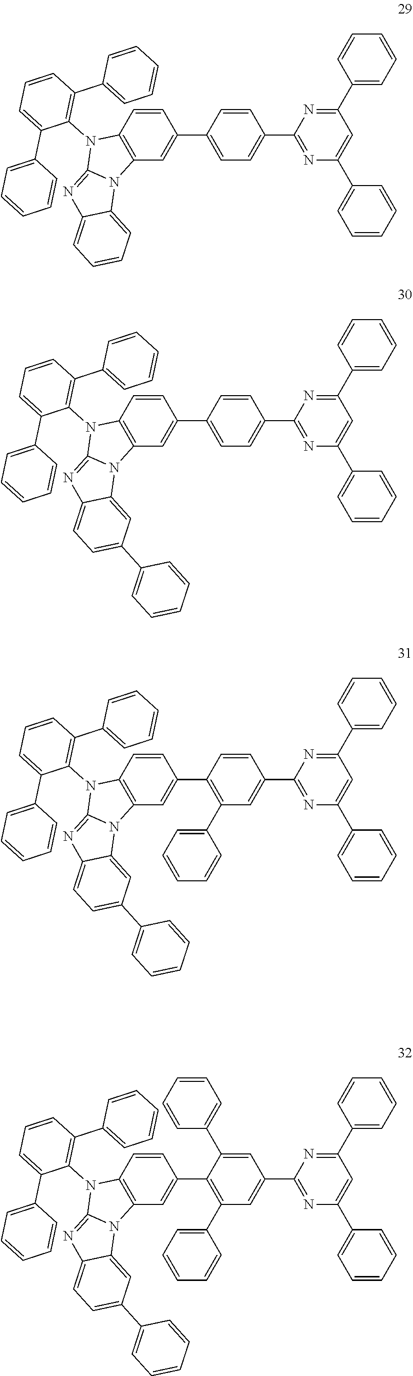 [SMILES: c1ccc(-c2cc(-c3ccccc3)nc(-c3ccc(-c4ccc5c(c4)n4c6ccccc6nc4n5-c4c(-c5ccccc5)cccc4-c4ccccc4)cc3)n2)cc1 ; c1ccc(-c2ccc3nc4n(-c5c(-c6ccccc6)cccc5-c5ccccc5)c5ccc(-c6ccc(-c7nc(-c8ccccc8)cc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7nc(-c8ccccc8)cc(-c8ccccc8)n7)cc6-c6ccccc6)cc5n4c3c2)cc1 ; c1ccc(-c2ccc3nc4n(-c5c(-c6ccccc6)cccc5-c5ccccc5)c5ccc(-c6c(-c7ccccc7)cc(-c7nc(-c8ccccc8)cc(-c8ccccc8)n7)cc6-c6ccccc6)cc5n4c3c2)cc1]

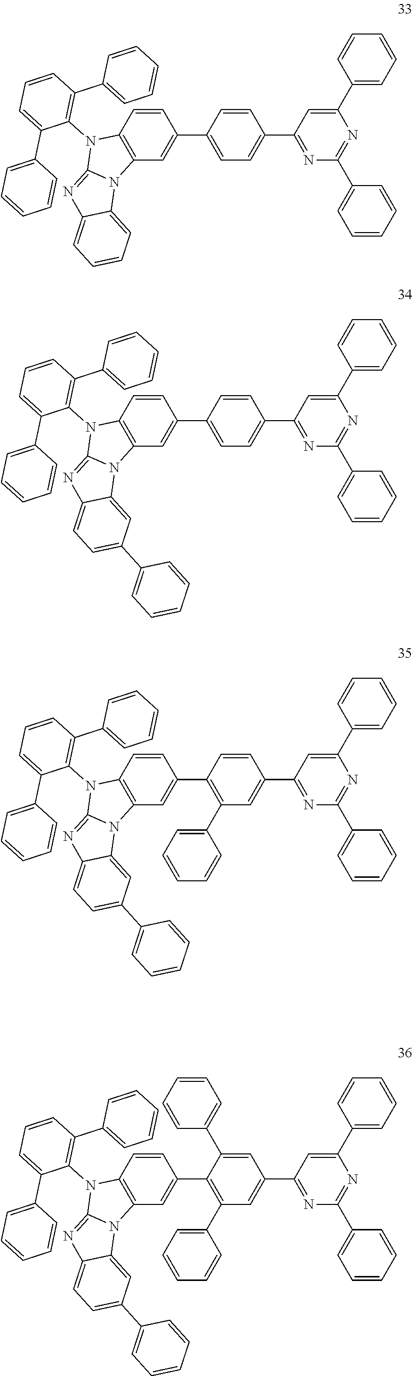 [SMILES: c1ccc(-c2cc(-c3ccc(-c4ccc5c(c4)n4c6ccccc6nc4n5-c4c(-c5ccccc5)cccc4-c4ccccc4)cc3)nc(-c3ccccc3)n2)cc1 ; c1ccc(-c2ccc3nc4n(-c5c(-c6ccccc6)cccc5-c5ccccc5)c5ccc(-c6ccc(-c7cc(-c8ccccc8)nc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7cc(-c8ccccc8)nc(-c8ccccc8)n7)cc6-c6ccccc6)cc5n4c3c2)cc1 ; c1ccc(-c2ccc3nc4n(-c5c(-c6ccccc6)cccc5-c5ccccc5)c5ccc(-c6c(-c7ccccc7)cc(-c7cc(-c8ccccc8)nc(-c8ccccc8)n7)cc6-c6ccccc6)cc5n4c3c2)cc1]

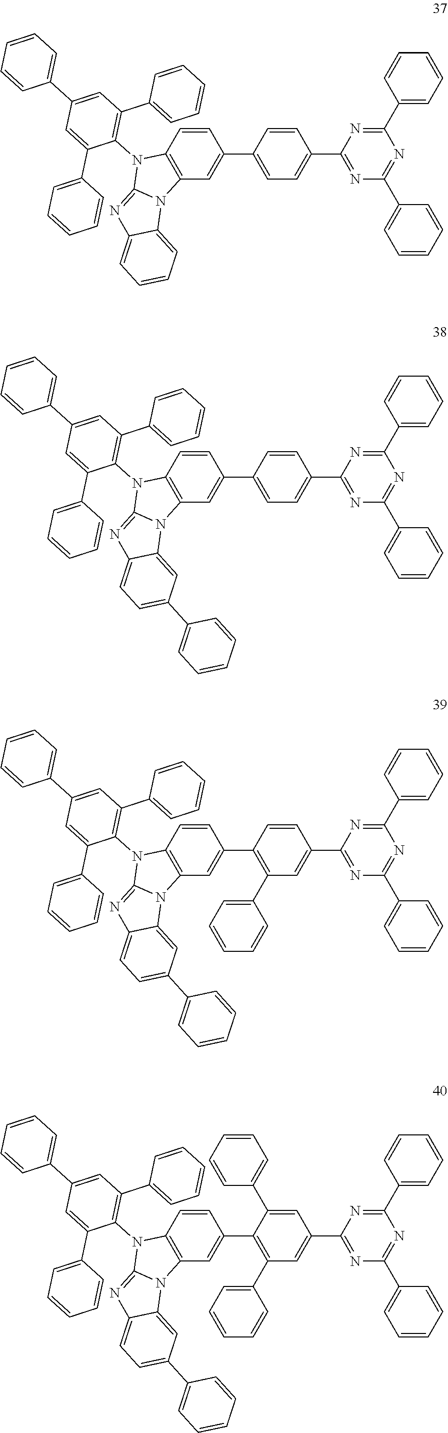

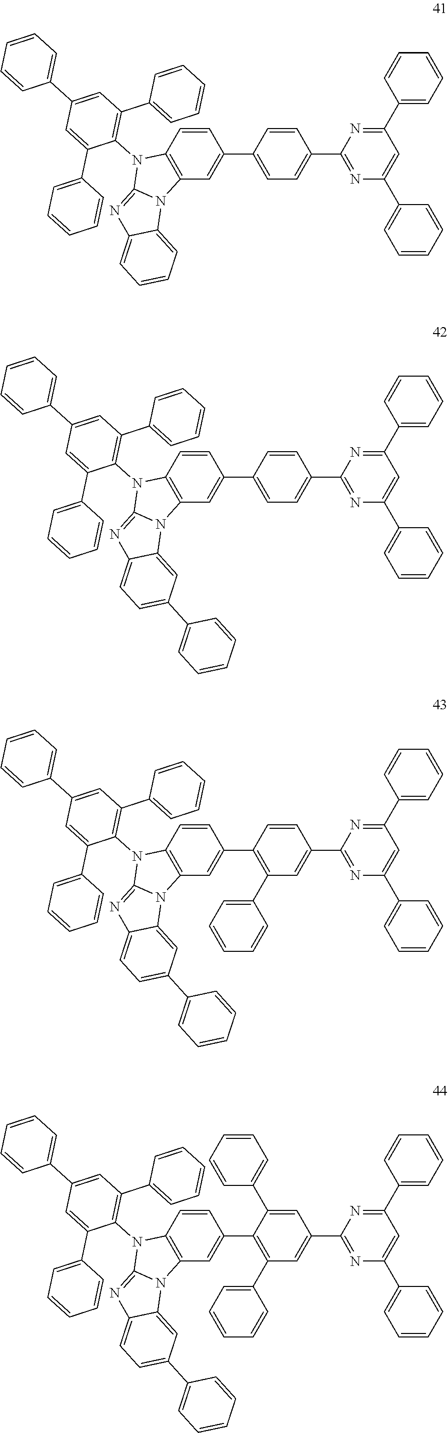

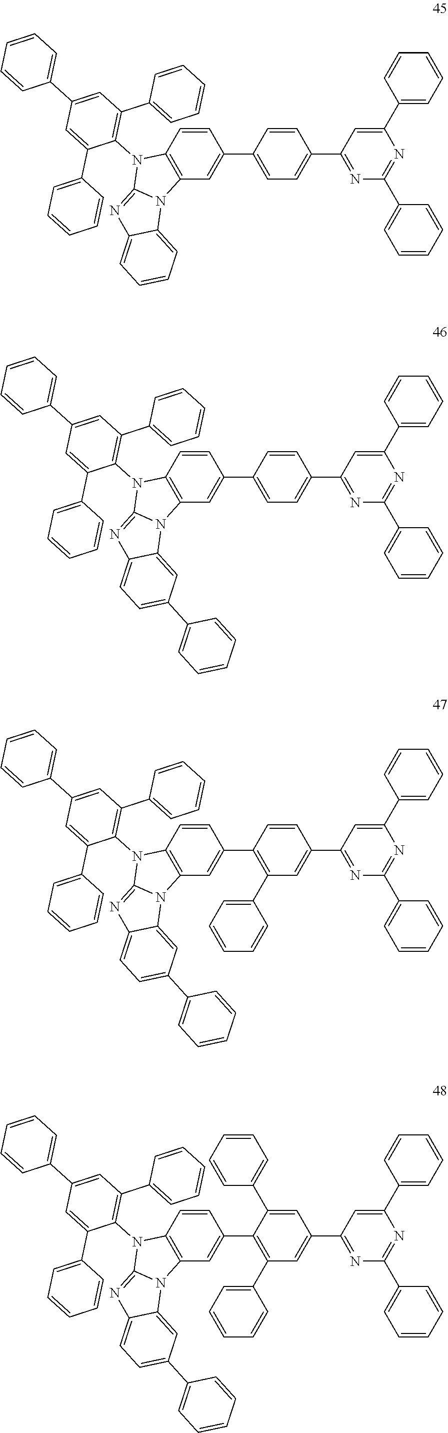

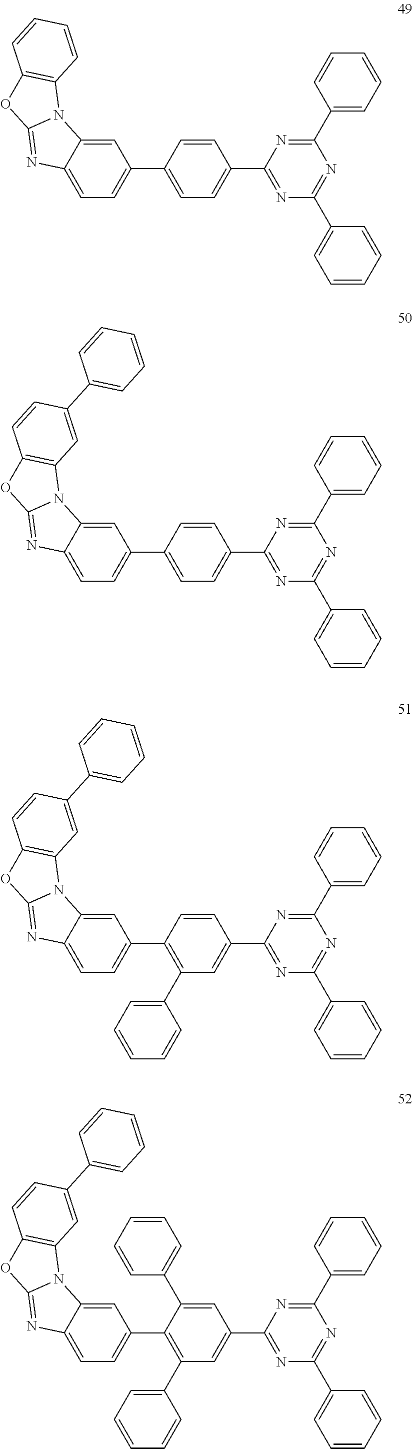 [SMILES: c1ccc(-c2nc(-c3ccccc3)nc(-c3ccc(-c4ccc5nc6oc7ccccc7n6c5c4)cc3)n2)cc1 ; c1ccc(-c2ccc3oc4nc5ccc(-c6ccc(-c7nc(-c8ccccc8)nc(-c8ccccc8)n7)cc6)cc5n4c3c2)cc1 ; c1ccc(-c2ccc3oc4nc5ccc(-c6ccc(-c7nc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7nc(-c8ccccc8)nc(-c8ccccc8)n7)cc6-c6ccccc6)cc5n4c3c2)cc1]

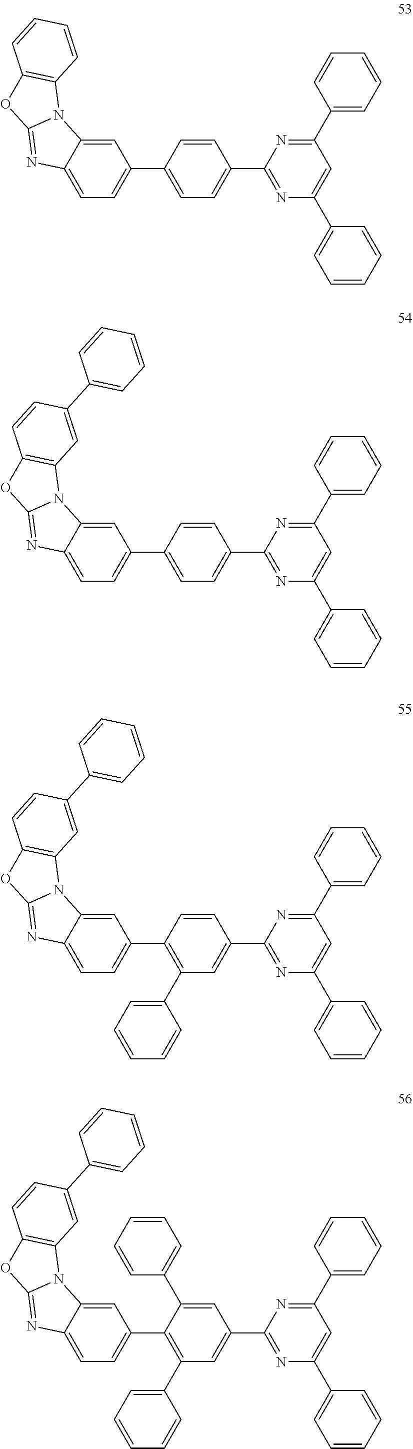 [SMILES: c1ccc(-c2cc(-c3ccccc3)nc(-c3ccc(-c4ccc5nc6oc7ccccc7n6c5c4)cc3)n2)cc1 ; c1ccc(-c2ccc3oc4nc5ccc(-c6ccc(-c7nc(-c8ccccc8)cc(-c8ccccc8)n7)cc6)cc5n4c3c2)cc1 ; c1ccc(-c2ccc3oc4nc5ccc(-c6ccc(-c7nc(-c8ccccc8)cc(-c8ccccc8)n7)cc6-c6ccccc6)cc5n4c3c2)cc1 ; c1ccc(-c2ccc3oc4nc5ccc(-c6c(-c7ccccc7)cc(-c7nc(-c8ccccc8)cc(-c8ccccc8)n7)cc6-c6ccccc6)cc5n4c3c2)cc1]

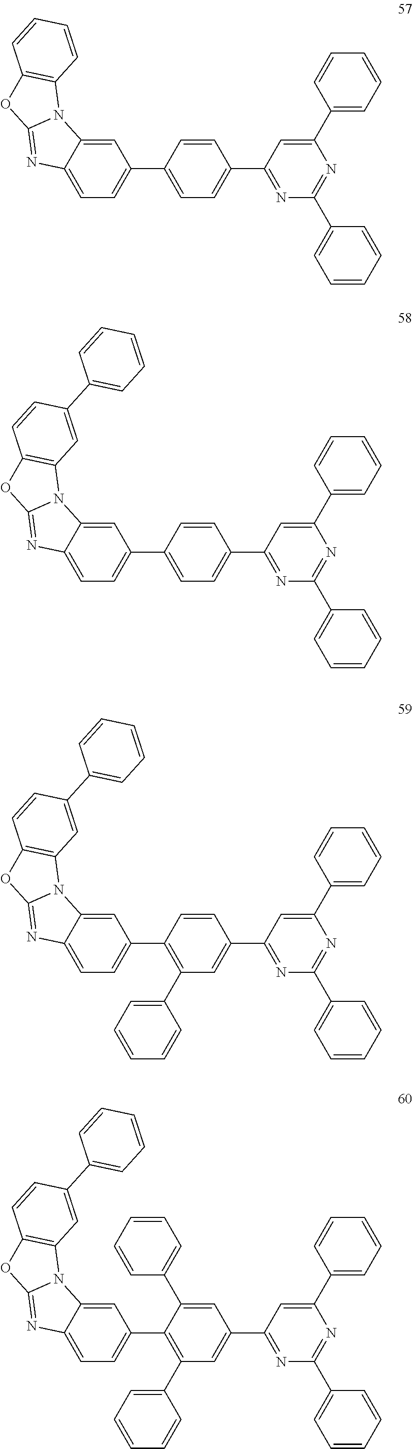 [SMILES: c1ccc(-c2cc(-c3ccc(-c4ccc5nc6oc7ccccc7n6c5c4)cc3)nc(-c3ccccc3)n2)cc1 ; c1ccc(-c2ccc3oc4nc5ccc(-c6ccc(-c7cc(-c8ccccc8)nc(-c8ccccc8)n7)cc6)cc5n4c3c2)cc1 ; c1ccc(-c2ccc3oc4nc5ccc(-c6ccc(-c7cc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7cc(-c8ccccc8)nc(-c8ccccc8)n7)cc6-c6ccccc6)cc5n4c3c2)cc1]

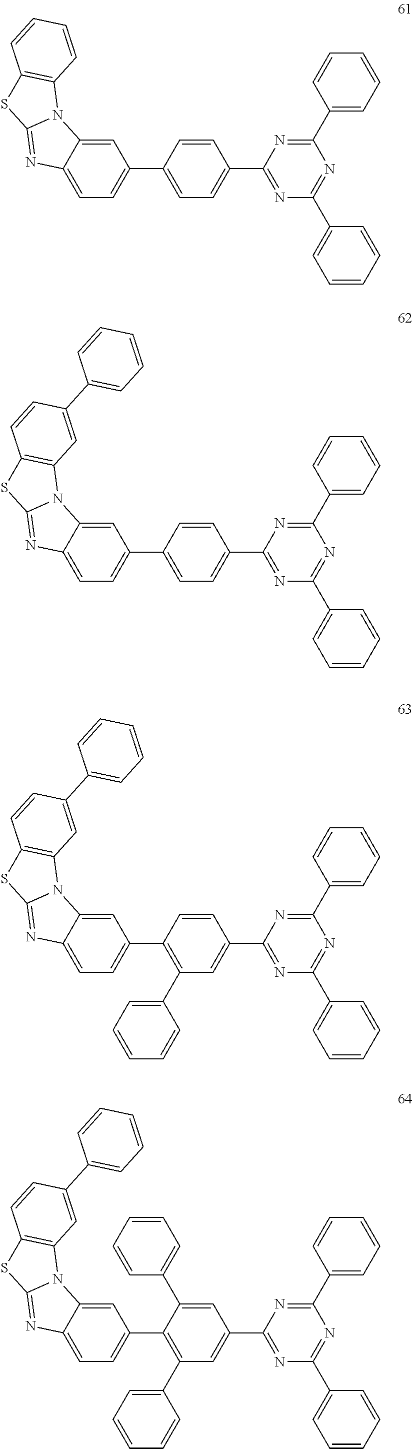 [SMILES: c1ccc(-c2nc(-c3ccccc3)nc(-c3ccc(-c4ccc5nc6sc7ccccc7n6c5c4)cc3)n2)cc1 ; c1ccc(-c2ccc3sc4nc5ccc(-c6ccc(-c7nc(-c8ccccc8)nc(-c8ccccc8)n7)cc6)cc5n4c3c2)cc1 ; c1ccc(-c2ccc3sc4nc5ccc(-c6ccc(-c7nc(-c8ccccc8)nc(-c8ccccc8)n7)cc6-c6ccccc6)cc5n4c3c2)cc1 ; c1ccc(-c2ccc3sc4nc5ccc(-c6c(-c7ccccc7)cc(-c7nc(-c8ccccc8)nc(-c8ccccc8)n7)cc6-c6ccccc6)cc5n4c3c2)cc1]

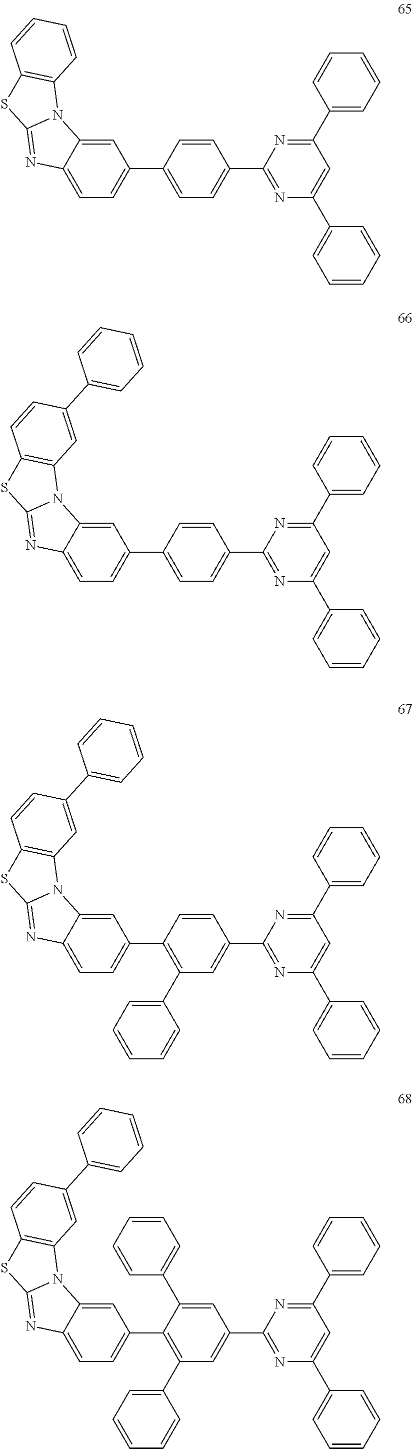 [SMILES: c1ccc(-c2cc(-c3ccccc3)nc(-c3ccc(-c4ccc5nc6sc7ccccc7n6c5c4)cc3)n2)cc1 ; c1ccc(-c2ccc3sc4nc5ccc(-c6ccc(-c7nc(-c8ccccc8)cc(-c8ccccc8)n7)cc6)cc5n4c3c2)cc1 ; c1ccc(-c2ccc3sc4nc5ccc(-c6ccc(-c7nc(-c8ccccc8)cc(-c8ccccc8)n7)cc6-c6ccccc6)cc5n4c3c2)cc1 ; c1ccc(-c2ccc3sc4nc5ccc(-c6c(-c7ccccc7)cc(-c7nc(-c8ccccc8)cc(-c8ccccc8)n7)cc6-c6ccccc6)cc5n4c3c2)cc1]

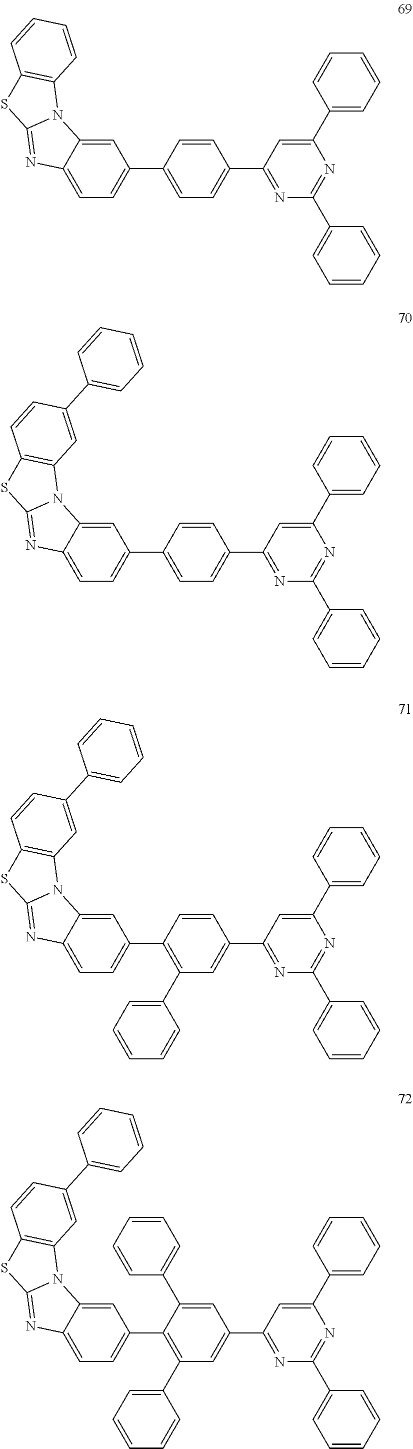 [SMILES: c1ccc(-c2cc(-c3ccc(-c4ccc5nc6sc7ccccc7n6c5c4)cc3)nc(-c3ccccc3)n2)cc1 ; c1ccc(-c2ccc3sc4nc5ccc(-c6ccc(-c7cc(-c8ccccc8)nc(-c8ccccc8)n7)cc6)cc5n4c3c2)cc1 ; c1ccc(-c2ccc3sc4nc5ccc(-c6ccc(-c7cc(-c8ccccc8)nc(-c8ccccc8)n7)cc6-c6ccccc6)cc5n4c3c2)cc1 ; c1ccc(-c2ccc3sc4nc5ccc(-c6c(-c7ccccc7)cc(-c7cc(-c8ccccc8)nc(-c8ccccc8)n7)cc6-c6ccccc6)cc5n4c3c2)cc1]

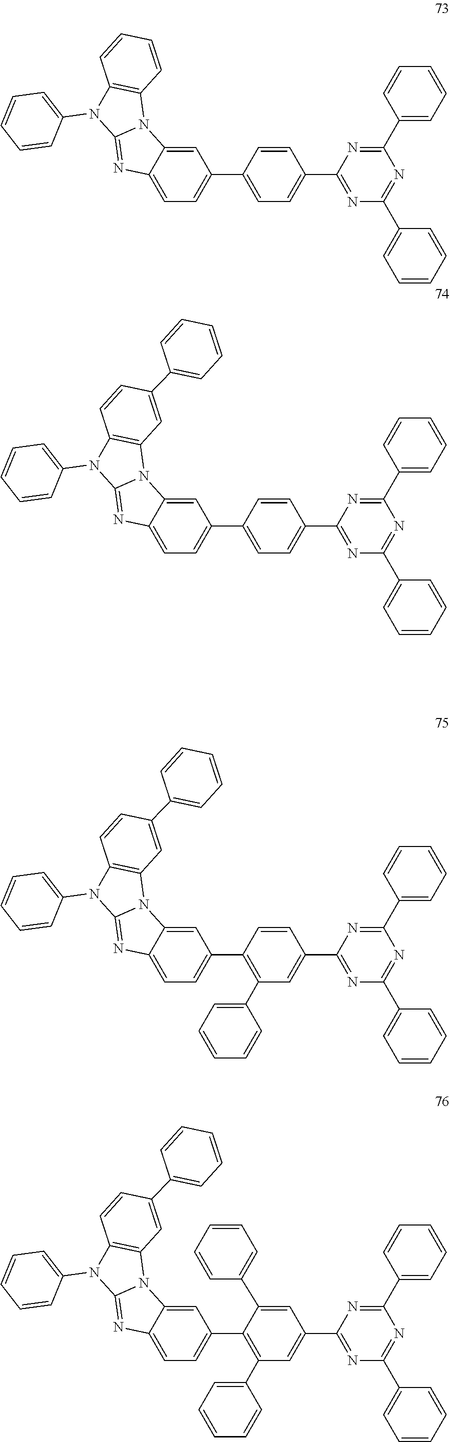 [SMILES: c1ccc(-c2nc(-c3ccccc3)nc(-c3ccc(-c4ccc5nc6n(-c7ccccc7)c7ccccc7n6c5c4)cc3)n2)cc1 ; c1ccc(-c2ccc3c(c2)n2c4cc(-c5ccc(-c6nc(-c7ccccc7)nc(-c7ccccc7)n6)cc5)ccc4nc2n3-c2ccccc2)cc1 ; c1ccc(-c2ccc3c(c2)n2c4cc(-c5ccc(-c6nc(-c7ccccc7)nc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6nc(-c7ccccc7)nc(-c7ccccc7)n6)cc5-c5ccccc5)ccc4nc2n3-c2ccccc2)cc1]

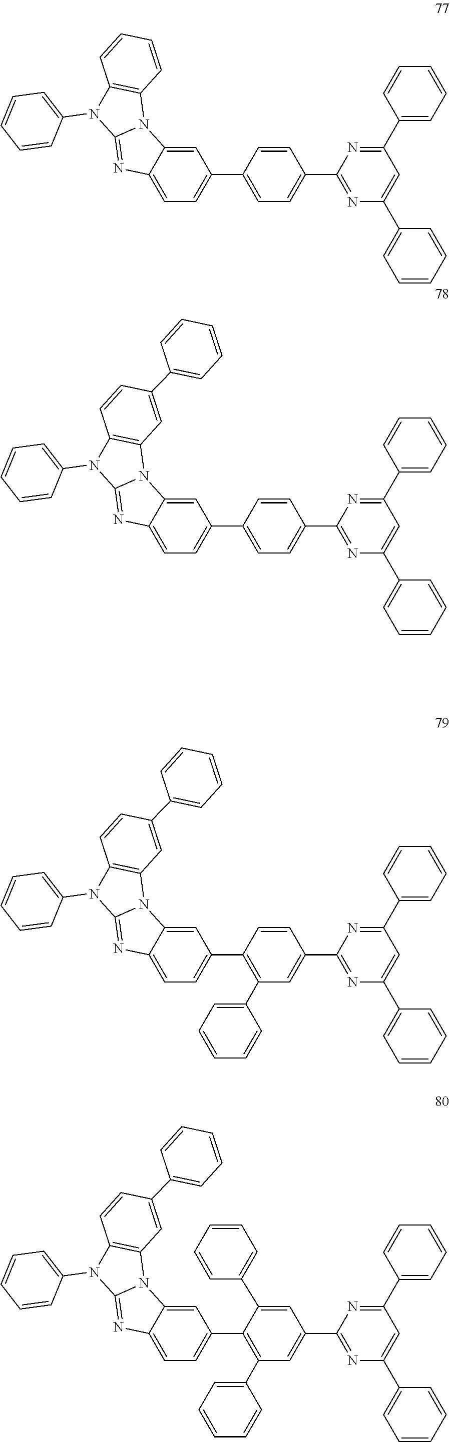 [SMILES: c1ccc(-c2cc(-c3ccccc3)nc(-c3ccc(-c4ccc5nc6n(-c7ccccc7)c7ccccc7n6c5c4)cc3)n2)cc1 ; c1ccc(-c2ccc3c(c2)n2c4cc(-c5ccc(-c6nc(-c7ccccc7)cc(-c7ccccc7)n6)cc5)ccc4nc2n3-c2ccccc2)cc1 ; c1ccc(-c2ccc3c(c2)n2c4cc(-c5ccc(-c6nc(-c7ccccc7)cc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6nc(-c7ccccc7)cc(-c7ccccc7)n6)cc5-c5ccccc5)ccc4nc2n3-c2ccccc2)cc1]

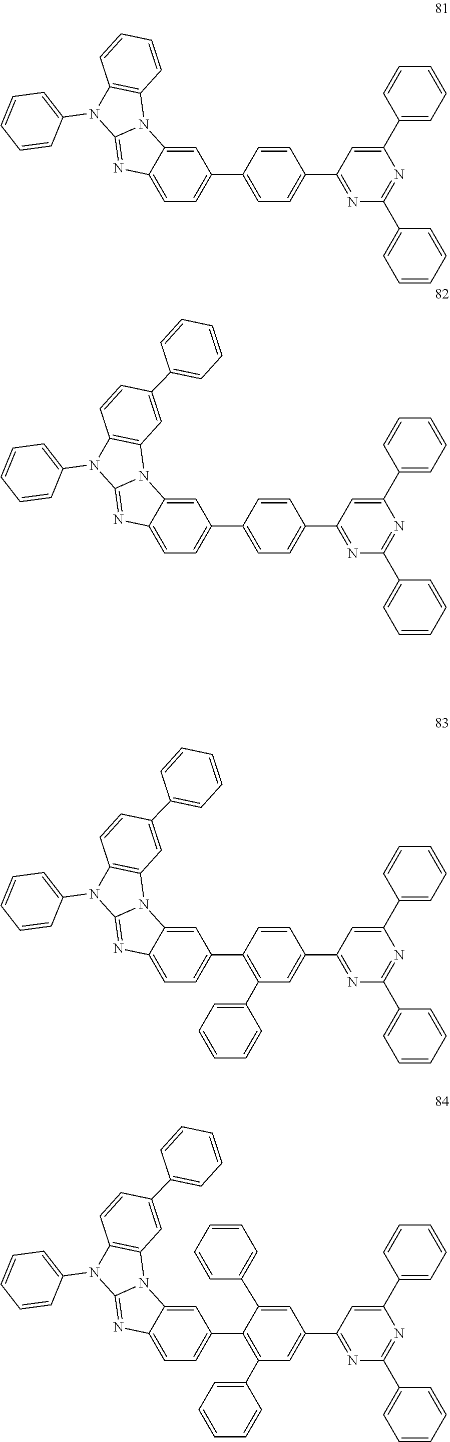 [SMILES: c1ccc(-c2cc(-c3ccc(-c4ccc5nc6n(-c7ccccc7)c7ccccc7n6c5c4)cc3)nc(-c3ccccc3)n2)cc1 ; c1ccc(-c2ccc3c(c2)n2c4cc(-c5ccc(-c6cc(-c7ccccc7)nc(-c7ccccc7)n6)cc5)ccc4nc2n3-c2ccccc2)cc1 ; c1ccc(-c2ccc3c(c2)n2c4cc(-c5ccc(-c6cc(-c7ccccc7)nc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6cc(-c7ccccc7)nc(-c7ccccc7)n6)cc5-c5ccccc5)ccc4nc2n3-c2ccccc2)cc1]

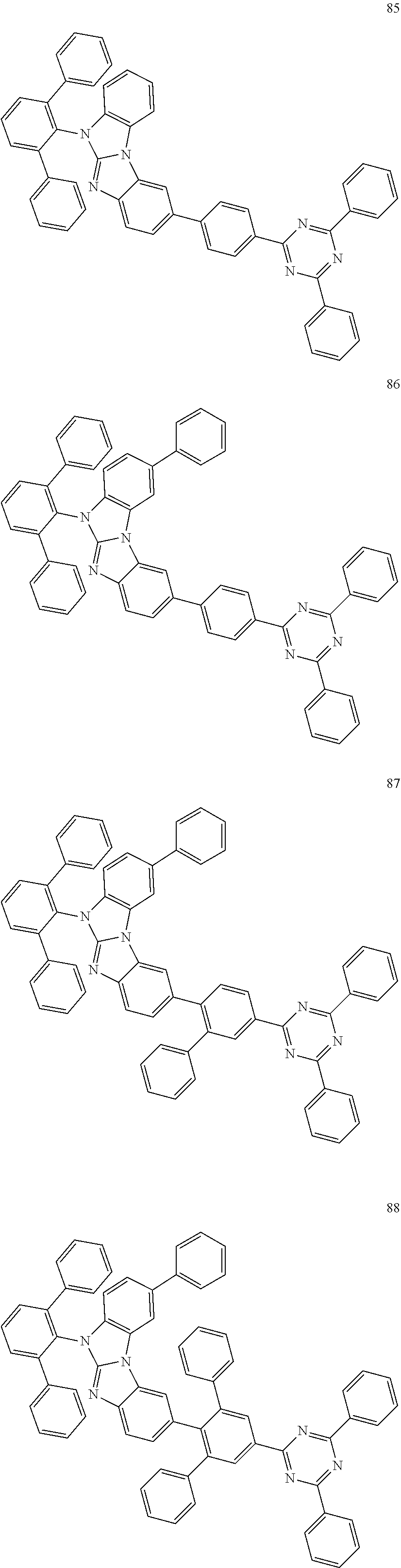 [SMILES: c1ccc(-c2nc(-c3ccccc3)nc(-c3ccc(-c4ccc5nc6n(-c7c(-c8ccccc8)cccc7-c7ccccc7)c7ccccc7n6c5c4)cc3)n2)cc1 ; c1ccc(-c2ccc3c(c2)n2c4cc(-c5ccc(-c6nc(-c7ccccc7)nc(-c7ccccc7)n6)cc5)ccc4nc2n3-c2c(-c3ccccc3)cccc2-c2ccccc2)cc1 ; c1ccc(-c2ccc3c(c2)n2c4cc(-c5ccc(-c6nc(-c7ccccc7)nc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6nc(-c7ccccc7)nc(-c7ccccc7)n6)cc5-c5ccccc5)ccc4nc2n3-c2c(-c3ccccc3)cccc2-c2ccccc2)cc1]

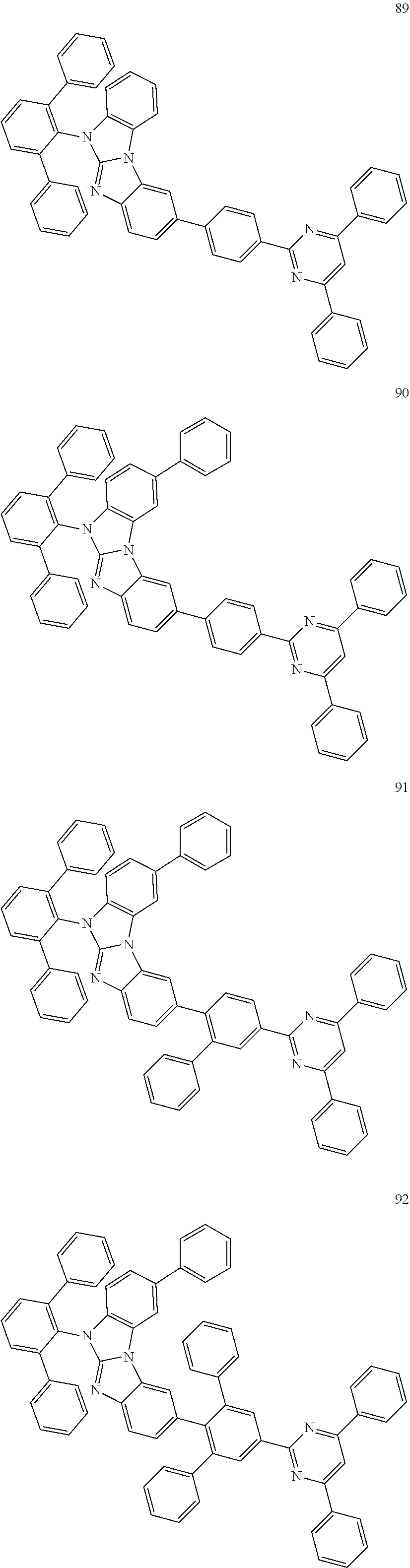 [SMILES: c1ccc(-c2cc(-c3ccccc3)nc(-c3ccc(-c4ccc5nc6n(-c7c(-c8ccccc8)cccc7-c7ccccc7)c7ccccc7n6c5c4)cc3)n2)cc1 ; c1ccc(-c2ccc3c(c2)n2c4cc(-c5ccc(-c6nc(-c7ccccc7)cc(-c7ccccc7)n6)cc5)ccc4nc2n3-c2c(-c3ccccc3)cccc2-c2ccccc2)cc1 ; c1ccc(-c2ccc3c(c2)n2c4cc(-c5ccc(-c6nc(-c7ccccc7)cc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6nc(-c7ccccc7)cc(-c7ccccc7)n6)cc5-c5ccccc5)ccc4nc2n3-c2c(-c3ccccc3)cccc2-c2ccccc2)cc1]

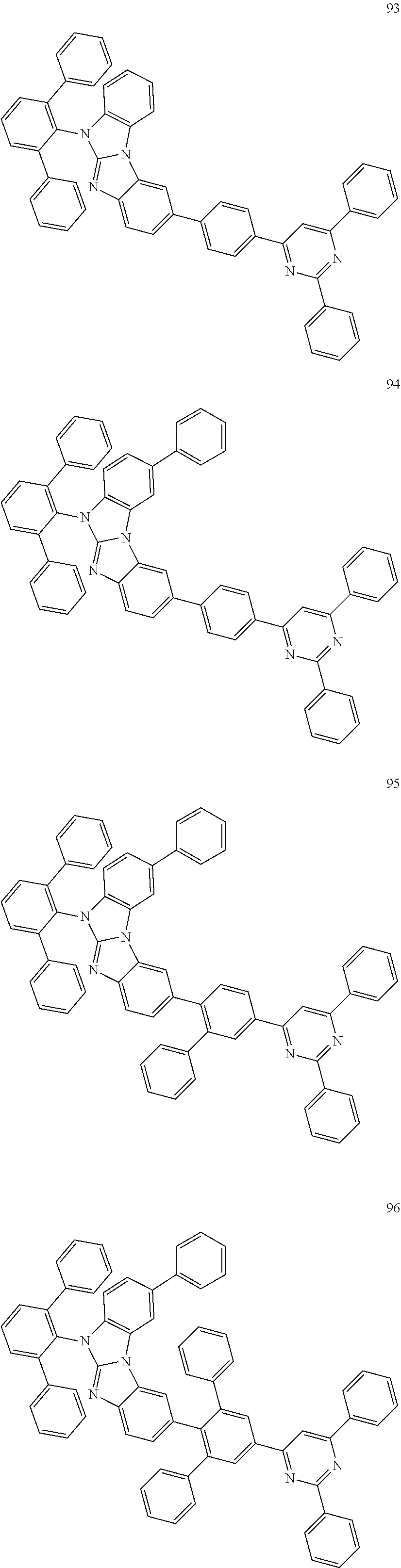 [SMILES: c1ccc(-c2cc(-c3ccc(-c4ccc5nc6n(-c7c(-c8ccccc8)cccc7-c7ccccc7)c7ccccc7n6c5c4)cc3)nc(-c3ccccc3)n2)cc1 ; c1ccc(-c2ccc3c(c2)n2c4cc(-c5ccc(-c6cc(-c7ccccc7)nc(-c7ccccc7)n6)cc5)ccc4nc2n3-c2c(-c3ccccc3)cccc2-c2ccccc2)cc1 ; c1ccc(-c2ccc3c(c2)n2c4cc(-c5ccc(-c6cc(-c7ccccc7)nc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6cc(-c7ccccc7)nc(-c7ccccc7)n6)cc5-c5ccccc5)ccc4nc2n3-c2c(-c3ccccc3)cccc2-c2ccccc2)cc1]

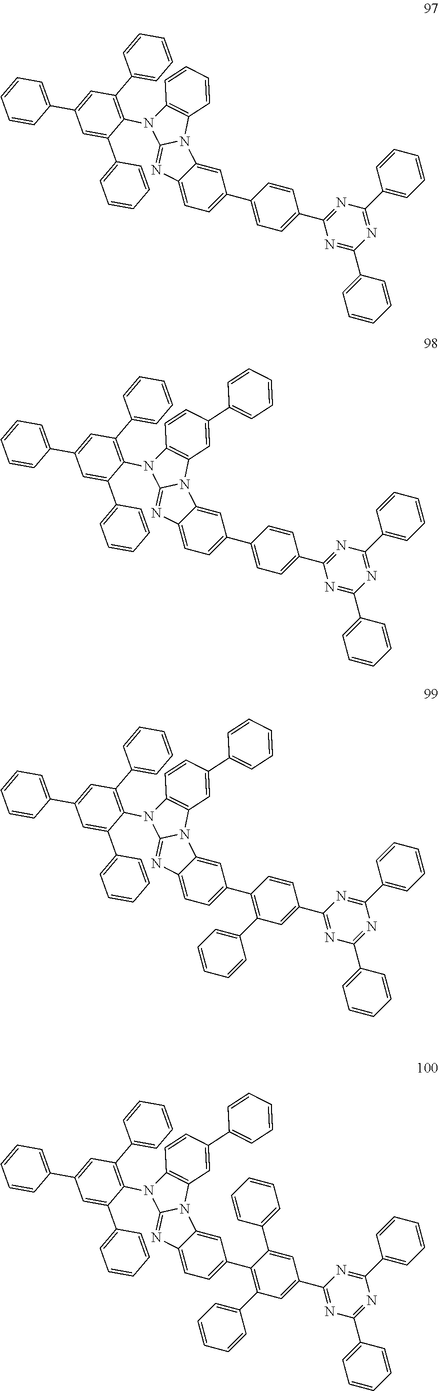

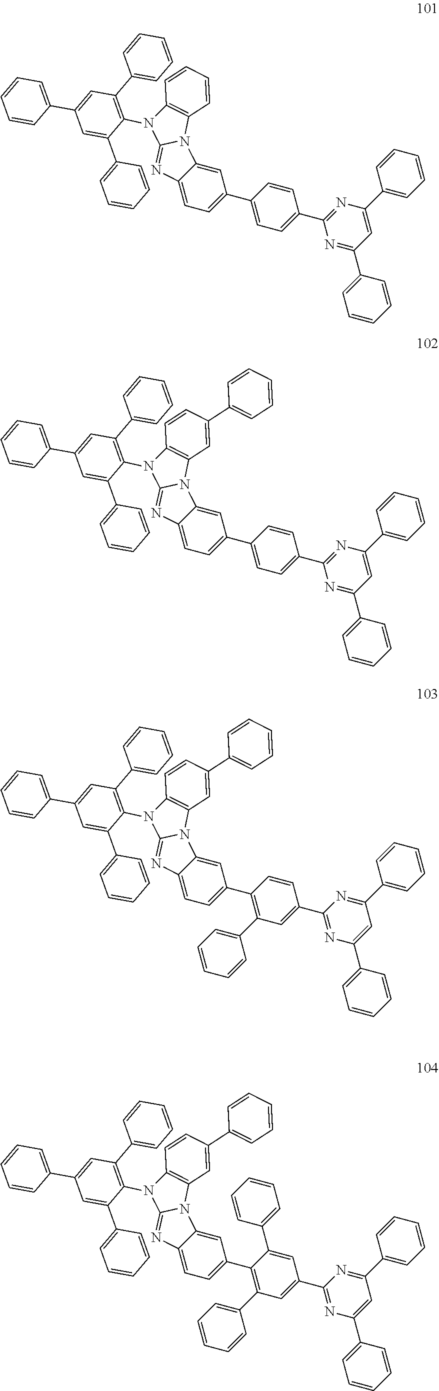

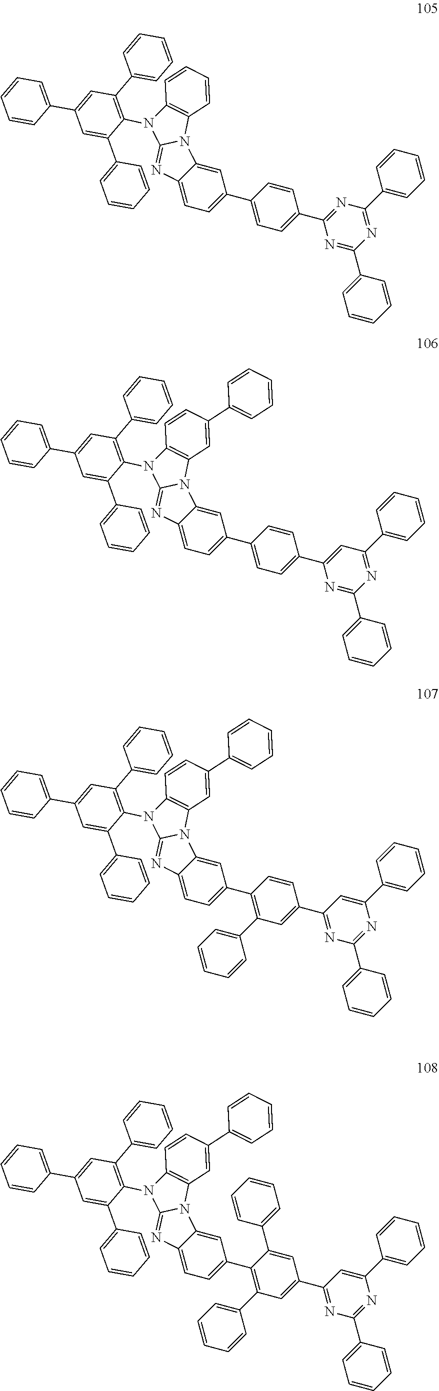

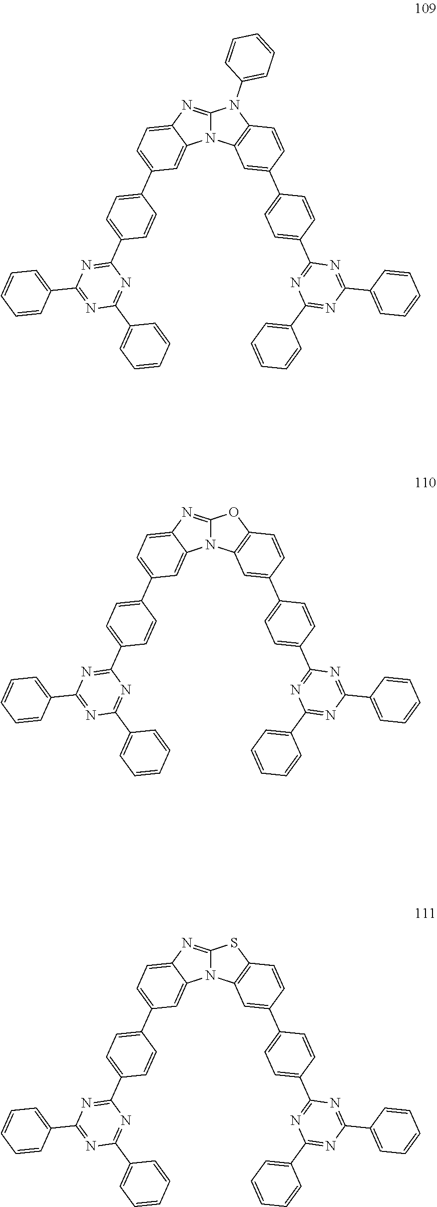 [SMILES: c1ccc(-c2nc(-c3ccccc3)nc(-c3ccc(-c4ccc5nc6n(-c7ccccc7)c7ccc(-c8ccc(-c9nc(-c%10ccccc%10)nc(-c%10ccccc%10)n9)cc8)cc7n6c5c4)cc3)n2)cc1 ; c1ccc(-c2nc(-c3ccccc3)nc(-c3ccc(-c4ccc5nc6oc7ccc(-c8ccc(-c9nc(-c%10ccccc%10)nc(-c%10ccccc%10)n9)cc8)cc7n6c5c4)cc3)n2)cc1 ; c1ccc(-c2nc(-c3ccccc3)nc(-c3ccc(-c4ccc5nc6sc7ccc(-c8ccc(-c9nc(-c%10ccccc%10)nc(-c%10ccccc%10)n9)cc8)cc7n6c5c4)cc3)n2)cc1]

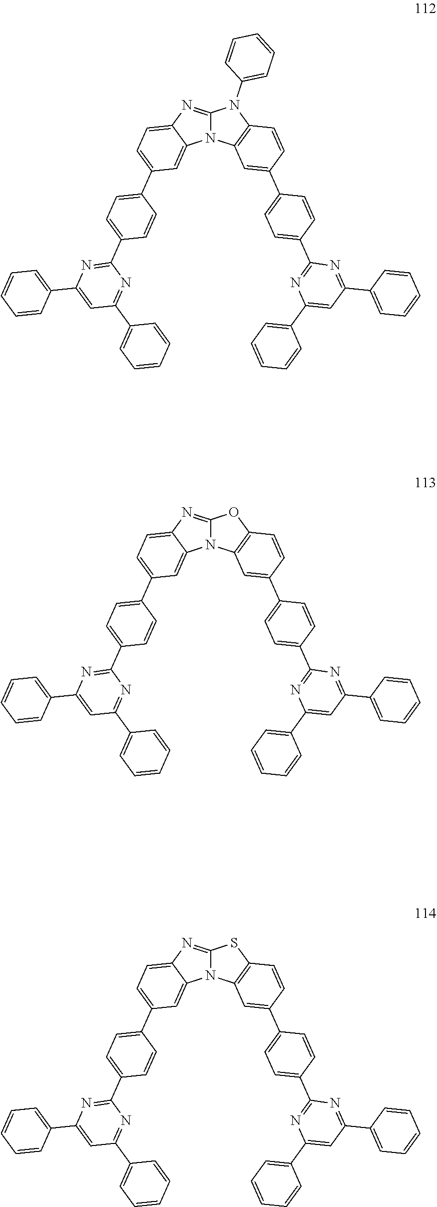 [SMILES: c1ccc(-c2cc(-c3ccccc3)nc(-c3ccc(-c4ccc5nc6n(-c7ccccc7)c7ccc(-c8ccc(-c9nc(-c%10ccccc%10)cc(-c%10ccccc%10)n9)cc8)cc7n6c5c4)cc3)n2)cc1 ; c1ccc(-c2cc(-c3ccccc3)nc(-c3ccc(-c4ccc5nc6oc7ccc(-c8ccc(-c9nc(-c%10ccccc%10)cc(-c%10ccccc%10)n9)cc8)cc7n6c5c4)cc3)n2)cc1 ; c1ccc(-c2cc(-c3ccccc3)nc(-c3ccc(-c4ccc5nc6sc7ccc(-c8ccc(-c9nc(-c%10ccccc%10)cc(-c%10ccccc%10)n9)cc8)cc7n6c5c4)cc3)n2)cc1]

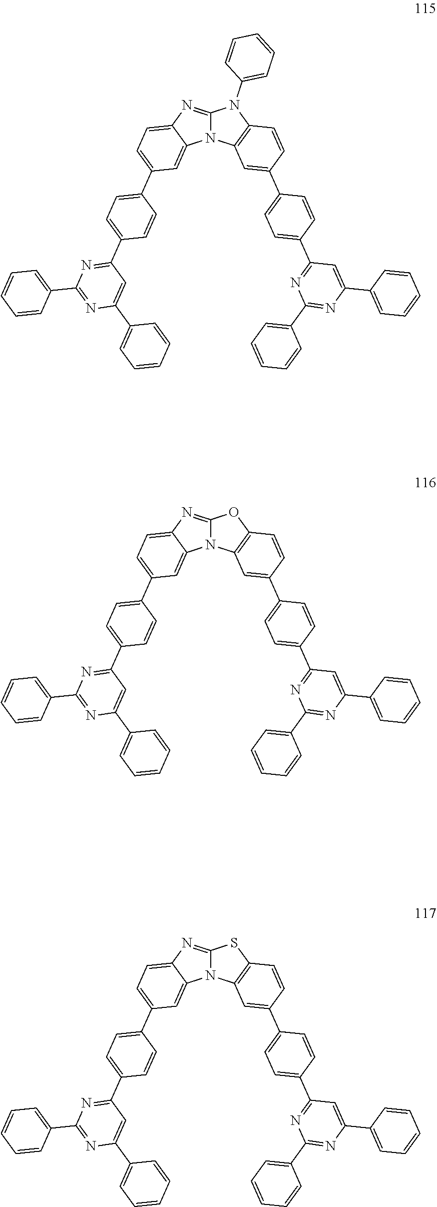 [SMILES: c1ccc(-c2cc(-c3ccc(-c4ccc5nc6n(-c7ccccc7)c7ccc(-c8ccc(-c9cc(-c%10ccccc%10)nc(-c%10ccccc%10)n9)cc8)cc7n6c5c4)cc3)nc(-c3ccccc3)n2)cc1 ; c1ccc(-c2cc(-c3ccc(-c4ccc5nc6oc7ccc(-c8ccc(-c9cc(-c%10ccccc%10)nc(-c%10ccccc%10)n9)cc8)cc7n6c5c4)cc3)nc(-c3ccccc3)n2)cc1 ; c1ccc(-c2cc(-c3ccc(-c4ccc5nc6sc7ccc(-c8ccc(-c9cc(-c%10ccccc%10)nc(-c%10ccccc%10)n9)cc8)cc7n6c5c4)cc3)nc(-c3ccccc3)n2)cc1]

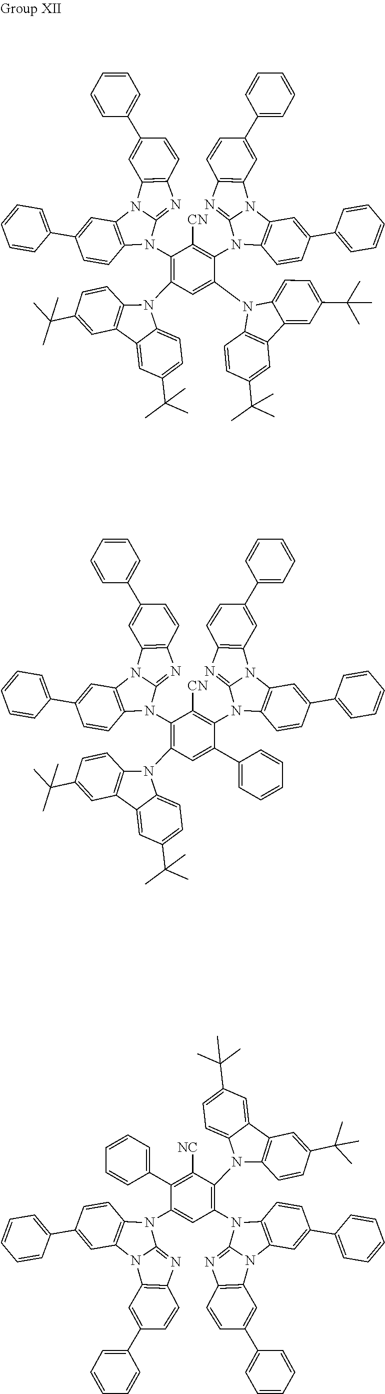

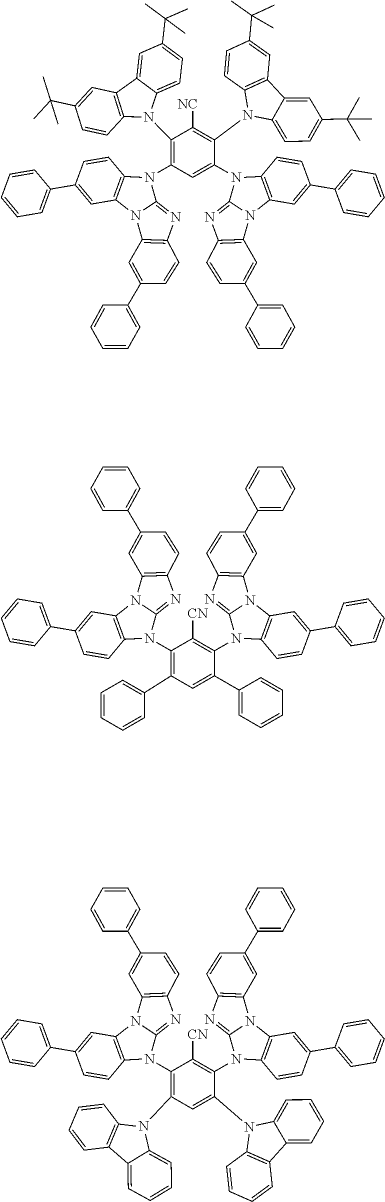 [SMILES: CC(C)(C)c1ccc2c(c1)c1cc(C(C)(C)C)ccc1n2-c1c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c(-n2c3ccc(C(C)(C)C)cc3c3cc(C(C)(C)C)ccc32)c1C#N ; N#Cc1c(-n2c3ccc(-c4ccccc4)cc3n3c4cc(-c5ccccc5)ccc4nc23)c(-c2ccccc2)cc(-c2ccccc2)c1-n1c2ccc(-c3ccccc3)cc2n2c3cc(-c4ccccc4)ccc3nc12 ; N#Cc1c(-n2c3ccc(-c4ccccc4)cc3n3c4cc(-c5ccccc5)ccc4nc23)c(-n2c3ccccc3c3ccccc32)cc(-n2c3ccccc3c3ccccc32)c1-n1c2ccc(-c3ccccc3)cc2n2c3cc(-c4ccccc4)ccc3nc12]

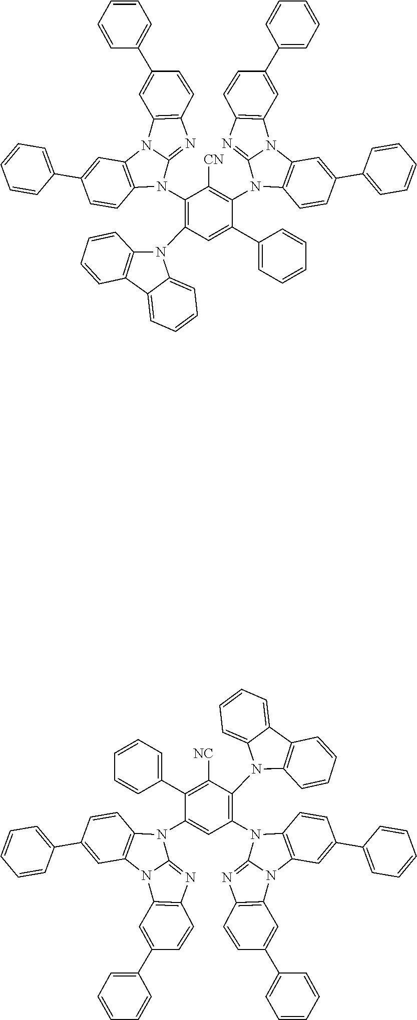 [SMILES: N#Cc1c(-n2c3ccc(-c4ccccc4)cc3n3c4cc(-c5ccccc5)ccc4nc23)c(-c2ccccc2)cc(-n2c3ccccc3c3ccccc32)c1-n1c2ccc(-c3ccccc3)cc2n2c3cc(-c4ccccc4)ccc3nc12 ; N#Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21]

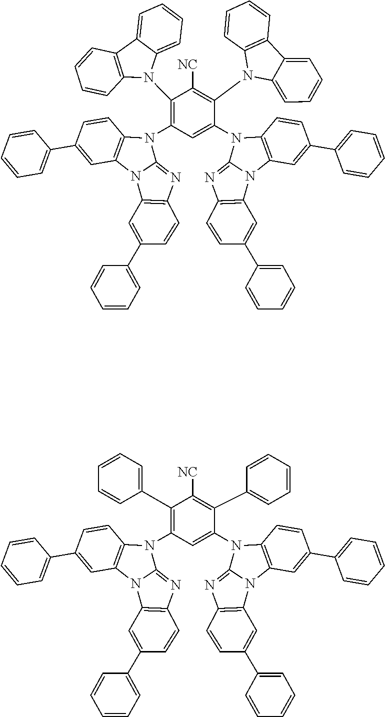 [SMILES: N#Cc1c(-n2c3ccccc3c3ccccc32)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21 ; N#Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-c1ccccc1]

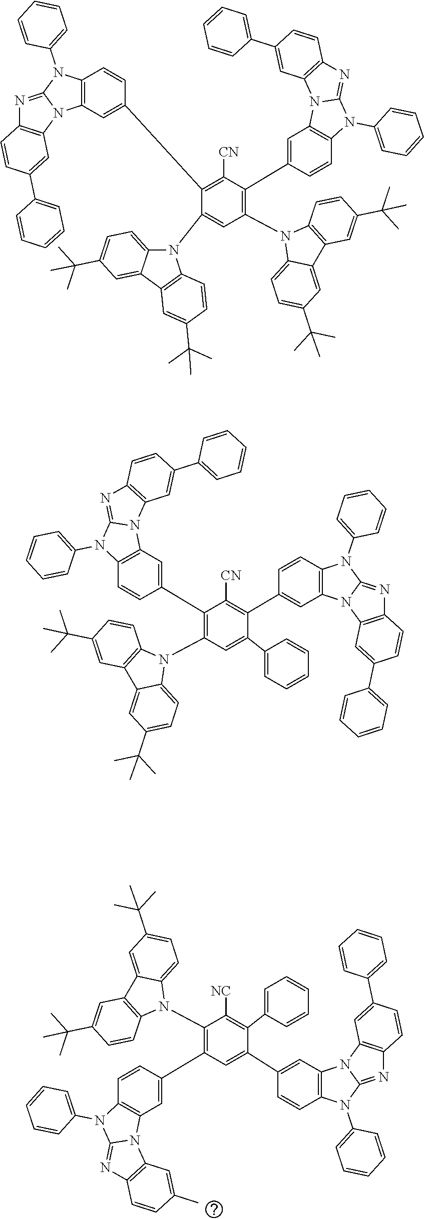

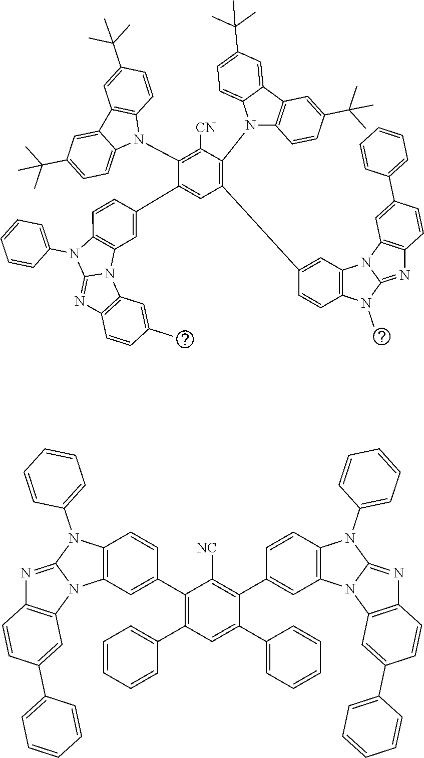 [SMILES: Cc1ccc2nc3n(-c4ccccc4)c4ccc(-c5cc(-c6ccc7c(c6)n6c8cc(-c9ccccc9)ccc8nc6n7C)c(-n6c7ccc(C(C)(C)C)cc7c7cc(C(C)(C)C)ccc76)c(C#N)c5-n5c6ccc(C(C)(C)C)cc6c6cc(C(C)(C)C)ccc65)cc4n3c2c1 ; N#Cc1c(-c2ccc3c(c2)n2c4cc(-c5ccccc5)ccc4nc2n3-c2ccccc2)c(-c2ccccc2)cc(-c2ccccc2)c1-c1ccc2c(c1)n1c3cc(-c4ccccc4)ccc3nc1n2-c1ccccc1]

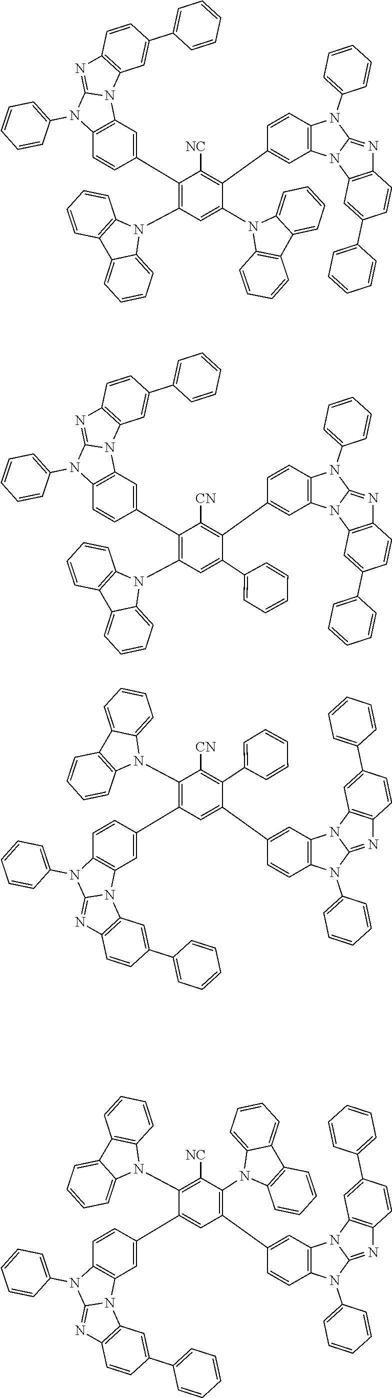

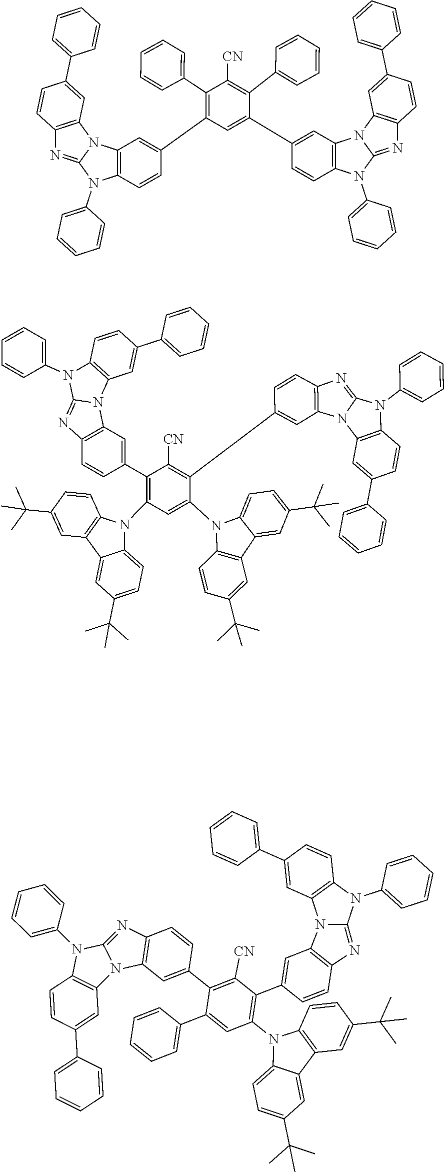

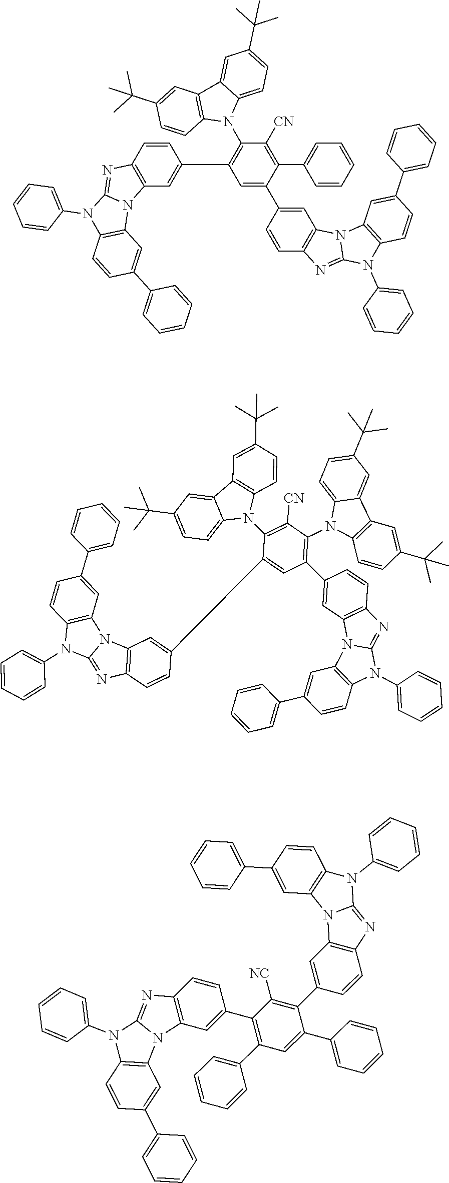

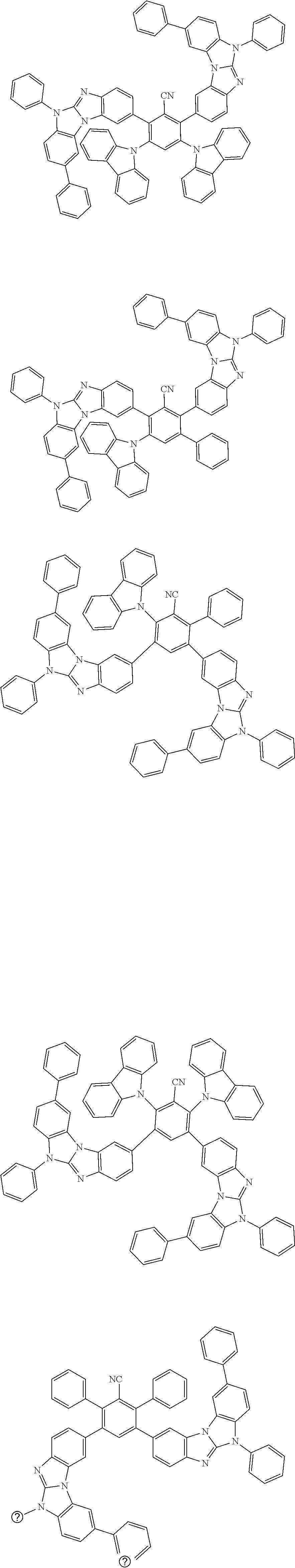

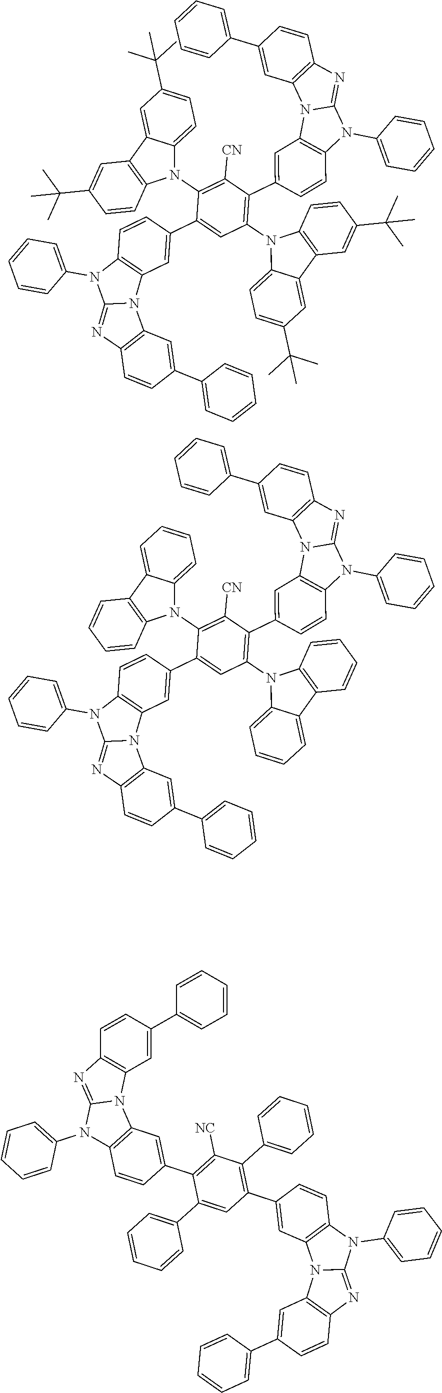

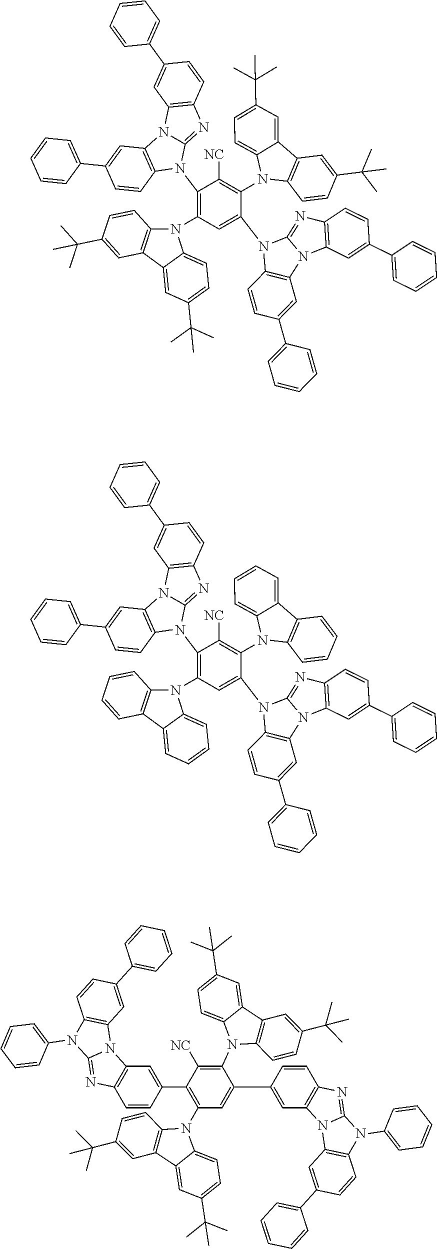

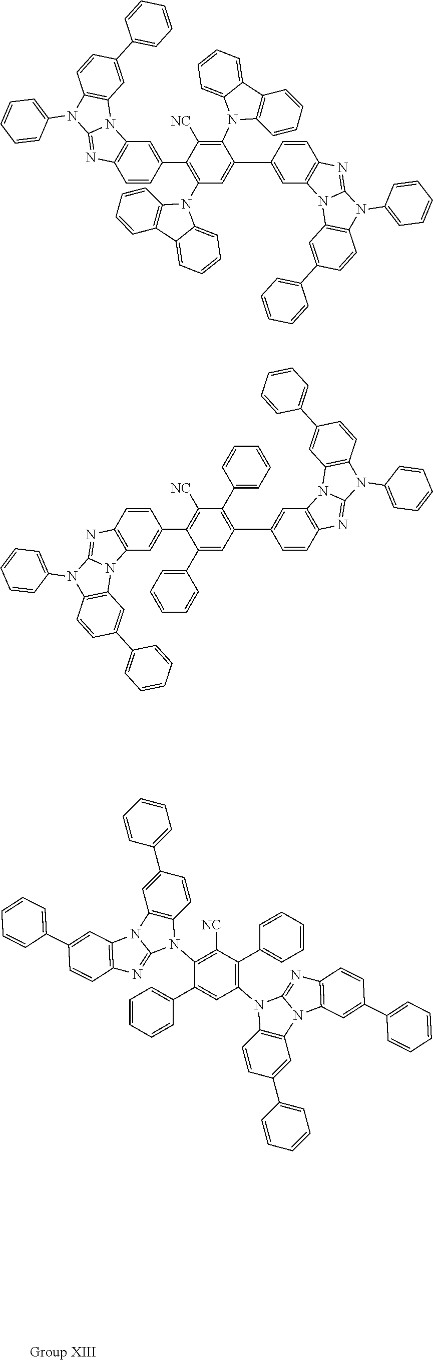

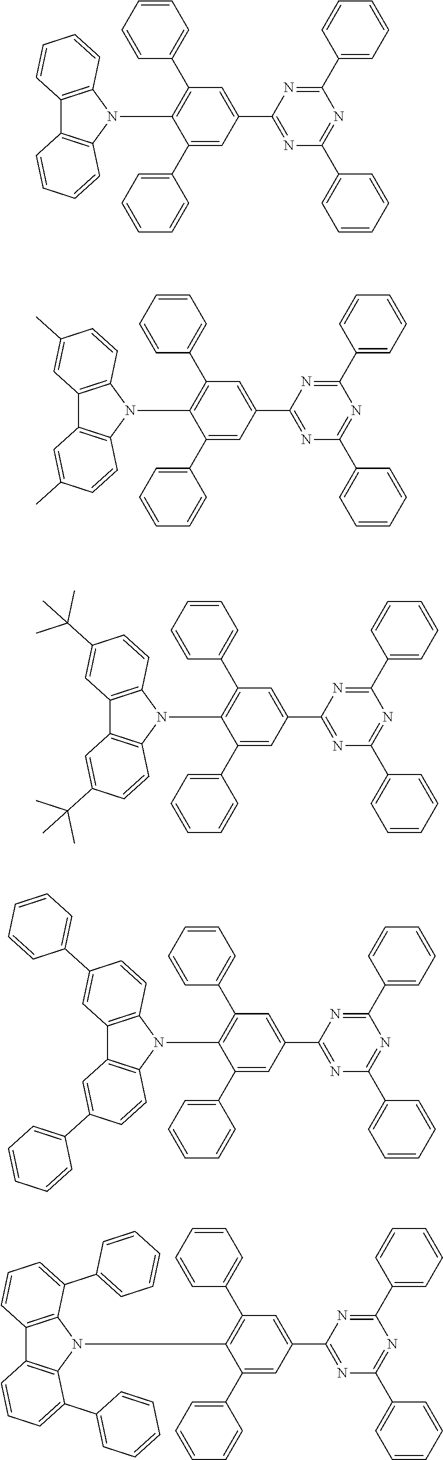 [SMILES: c1ccc(-c2nc(-c3ccccc3)nc(-c3cc(-c4ccccc4)c(-n4c5ccccc5c5ccccc54)c(-c4ccccc4)c3)n2)cc1 ; Cc1ccc2c(c1)c1cc(C)ccc1n2-c1c(-c2ccccc2)cc(-c2nc(-c3ccccc3)nc(-c3ccccc3)n2)cc1-c1ccccc1 ; CC(C)(C)c1ccc2c(c1)c1cc(C(C)(C)C)ccc1n2-c1c(-c2ccccc2)cc(-c2nc(-c3ccccc3)nc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)nc(-c4ccccc4)n3)cc2-c2ccccc2)cc1 ; c1ccc(-c2nc(-c3ccccc3)nc(-c3cc(-c4ccccc4)c(-n4c5c(-c6ccccc6)cccc5c5cccc(-c6ccccc6)c54)c(-c4ccccc4)c3)n2)cc1]

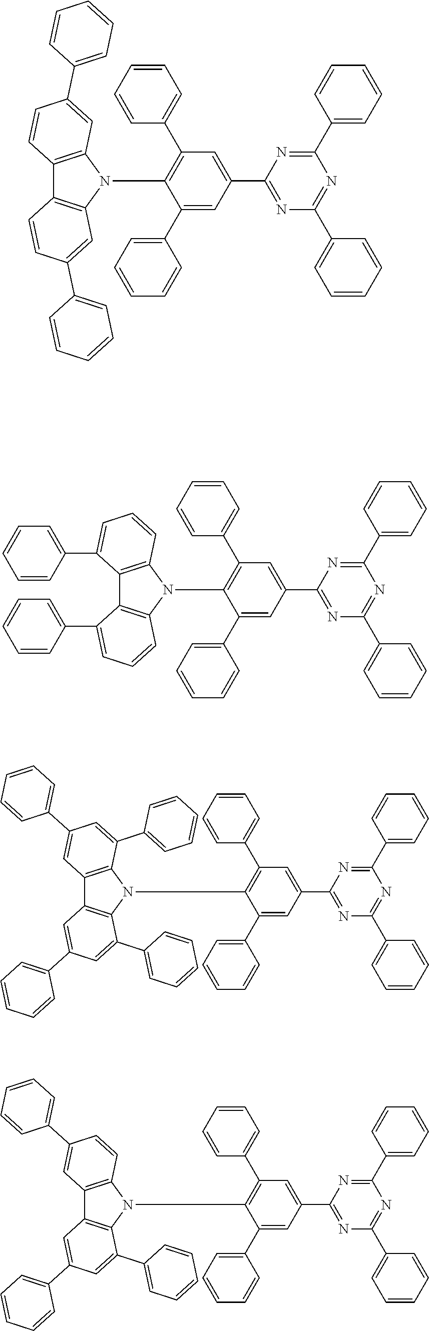 [SMILES: c1ccc(-c2ccc3c4ccc(-c5ccccc5)cc4n(-c4c(-c5ccccc5)cc(-c5nc(-c6ccccc6)nc(-c6ccccc6)n5)cc4-c4ccccc4)c3c2)cc1 ; c1ccc(-c2nc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1]

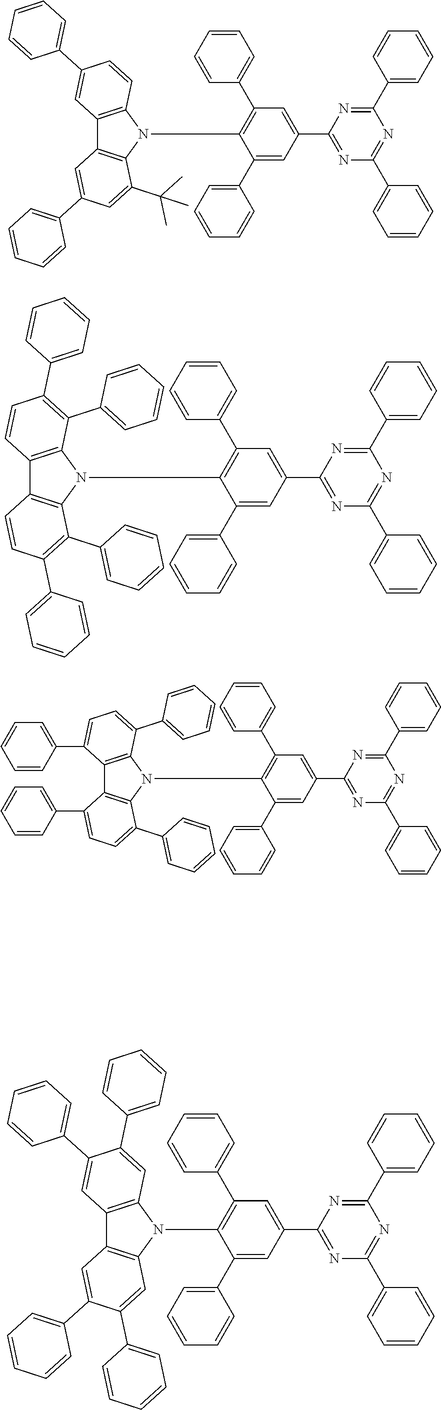

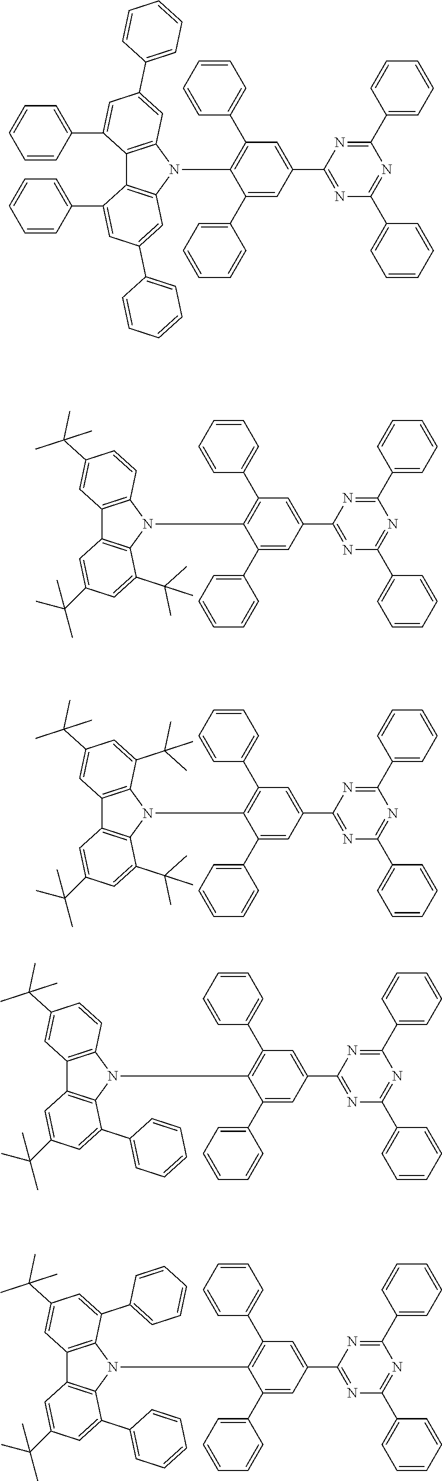

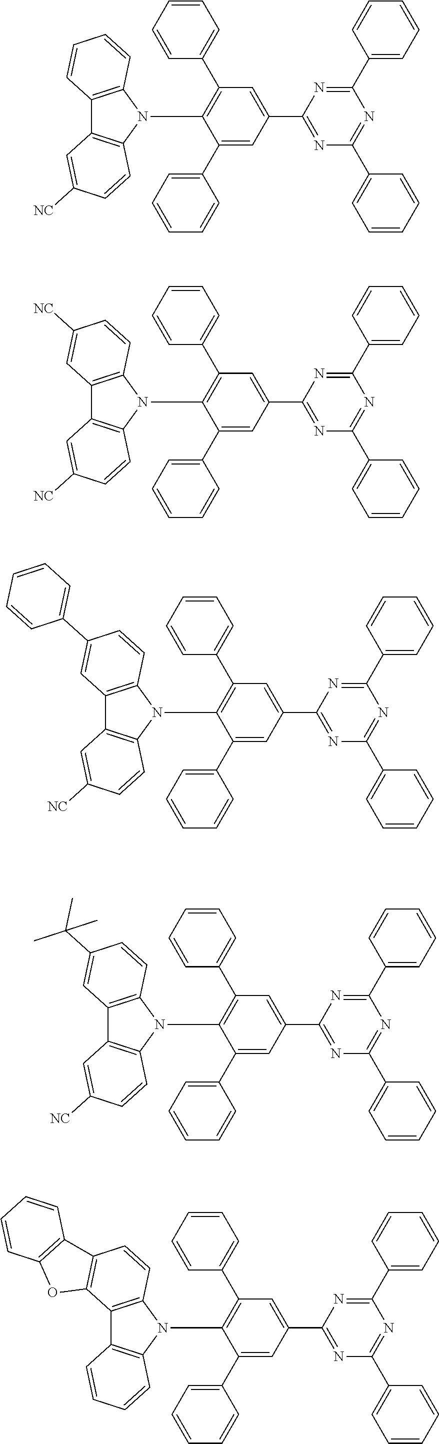 [SMILES: N#Cc1ccc2c(c1)c1ccccc1n2-c1c(-c2ccccc2)cc(-c2nc(-c3ccccc3)nc(-c3ccccc3)n2)cc1-c1ccccc1 ; N#Cc1ccc2c(c1)c1cc(C#N)ccc1n2-c1c(-c2ccccc2)cc(-c2nc(-c3ccccc3)nc(-c3ccccc3)n2)cc1-c1ccccc1 ; N#Cc1ccc2c(c1)c1cc(-c3ccccc3)ccc1n2-c1c(-c2ccccc2)cc(-c2nc(-c3ccccc3)nc(-c3ccccc3)n2)cc1-c1ccccc1 ; CC(C)(C)c1ccc2c(c1)c1cc(C#N)ccc1n2-c1c(-c2ccccc2)cc(-c2nc(-c3ccccc3)nc(-c3ccccc3)n2)cc1-c1ccccc1 ; c1ccc(-c2nc(-c3ccccc3)nc(-c3cc(-c4ccccc4)c(-n4c5ccccc5c5c6oc7ccccc7c6ccc54)c(-c4ccccc4)c3)n2)cc1]

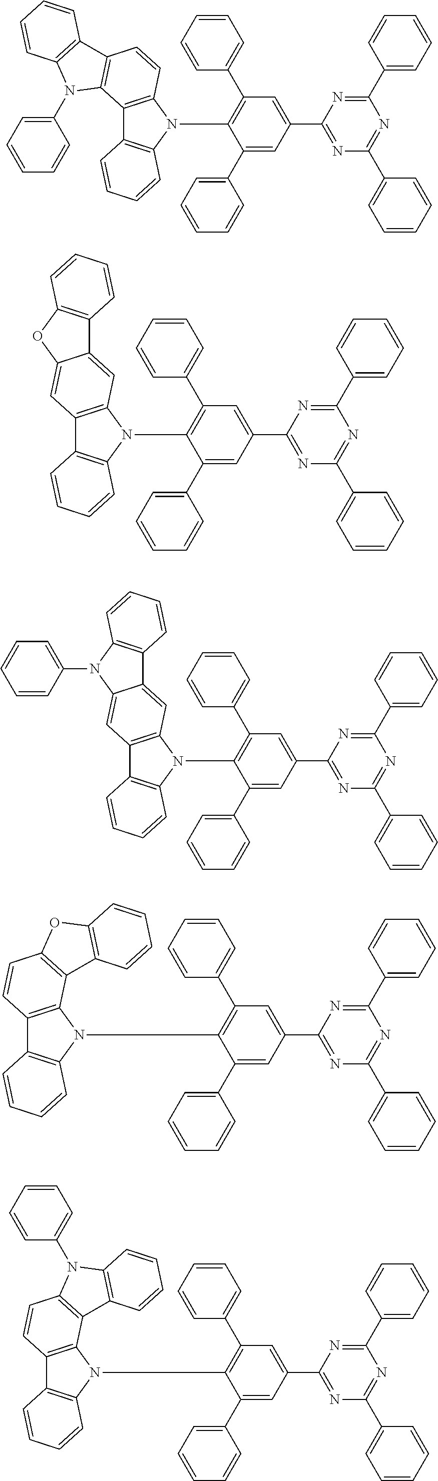

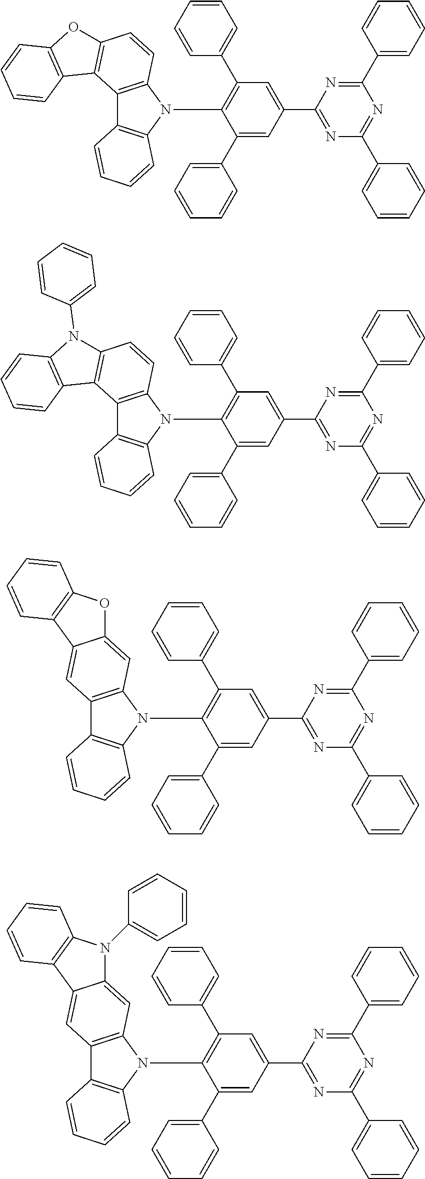

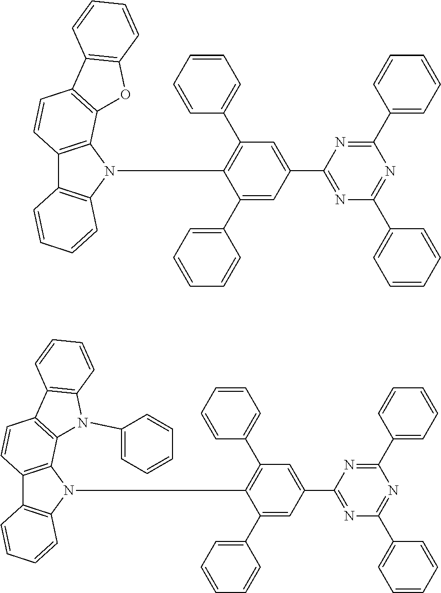 [SMILES: c1ccc(-c2nc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2nc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1]

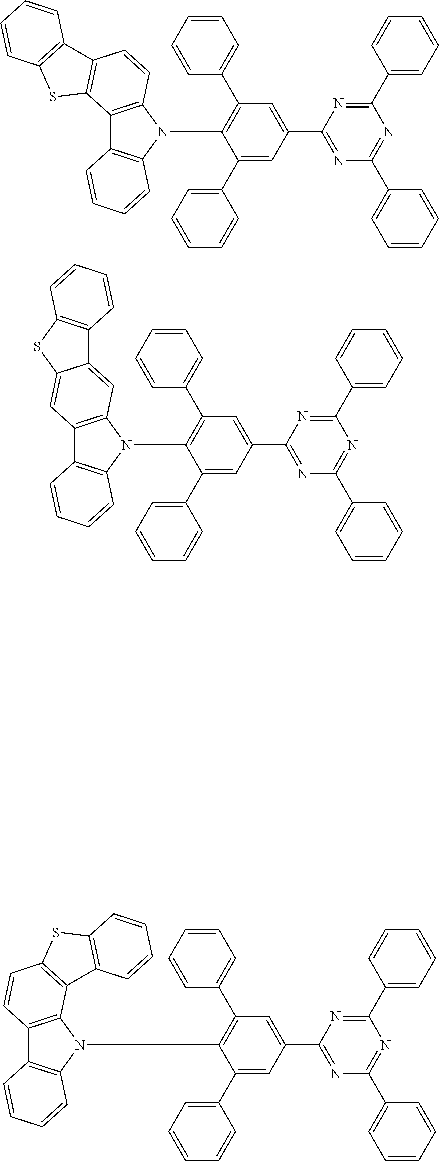 [SMILES: c1ccc(-c2nc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2nc(-c3ccccc3)nc(-c3cc(-c4ccccc4)c(-n4c5ccccc5c5cc6sc7ccccc7c6cc54)c(-c4ccccc4)c3)n2)cc1 ; c1ccc(-c2nc(-c3ccccc3)nc(-c3cc(-c4ccccc4)c(-n4c5ccccc5c5ccc6sc7ccccc7c6c54)c(-c4ccccc4)c3)n2)cc1]

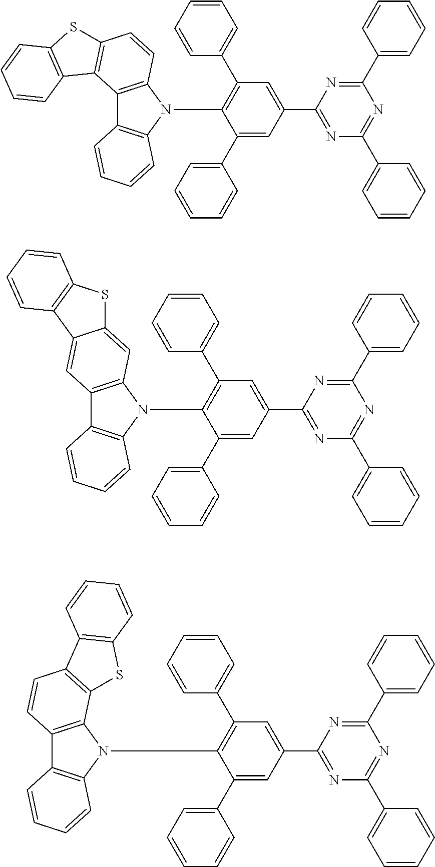 [SMILES: c1ccc(-c2nc(-c3ccccc3)nc(-c3cc(-c4ccccc4)c(-n4c5ccccc5c5c6c(ccc54)sc4ccccc46)c(-c4ccccc4)c3)n2)cc1 ; c1ccc(-c2nc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2nc(-c3ccccc3)nc(-c3cc(-c4ccccc4)c(-n4c5ccccc5c5ccc6c7ccccc7sc6c54)c(-c4ccccc4)c3)n2)cc1]

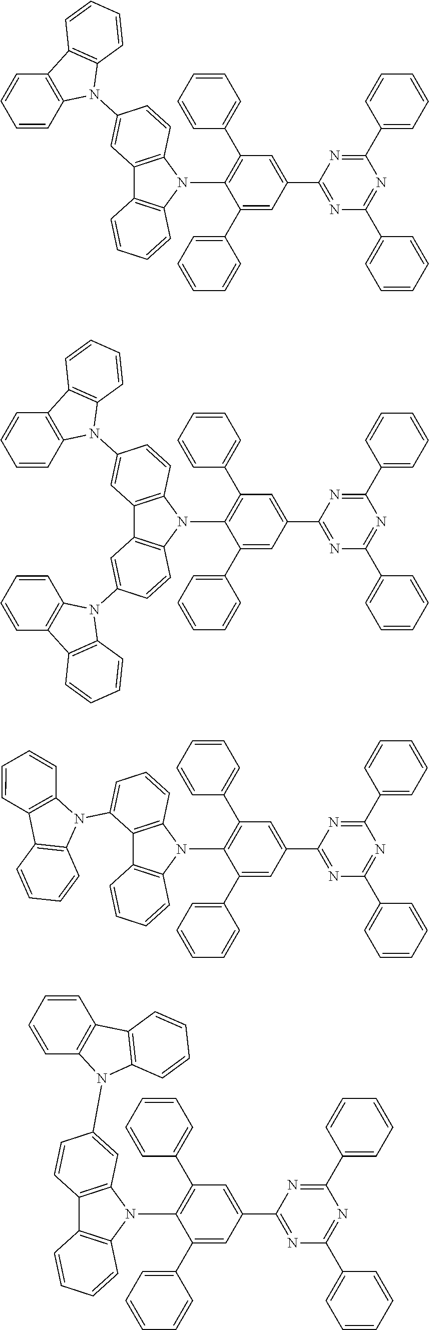 [SMILES: c1ccc(-c2nc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2nc(-c3ccccc3)nc(-c3cc(-c4ccccc4)c(-n4c5ccccc5c5ccc(-n6c7ccccc7c7ccccc76)cc54)c(-c4ccccc4)c3)n2)cc1]

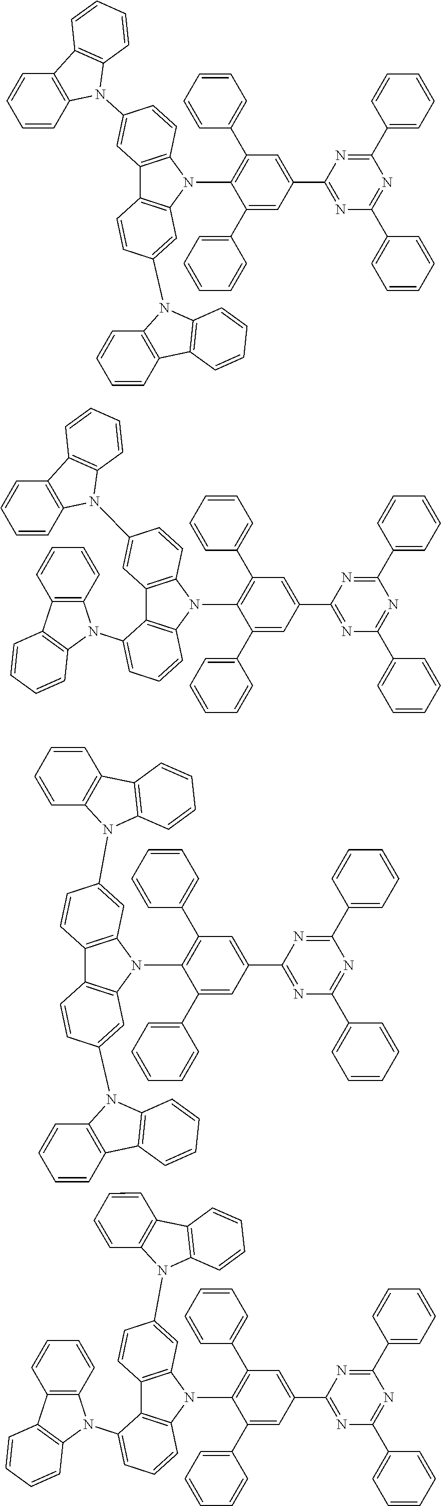

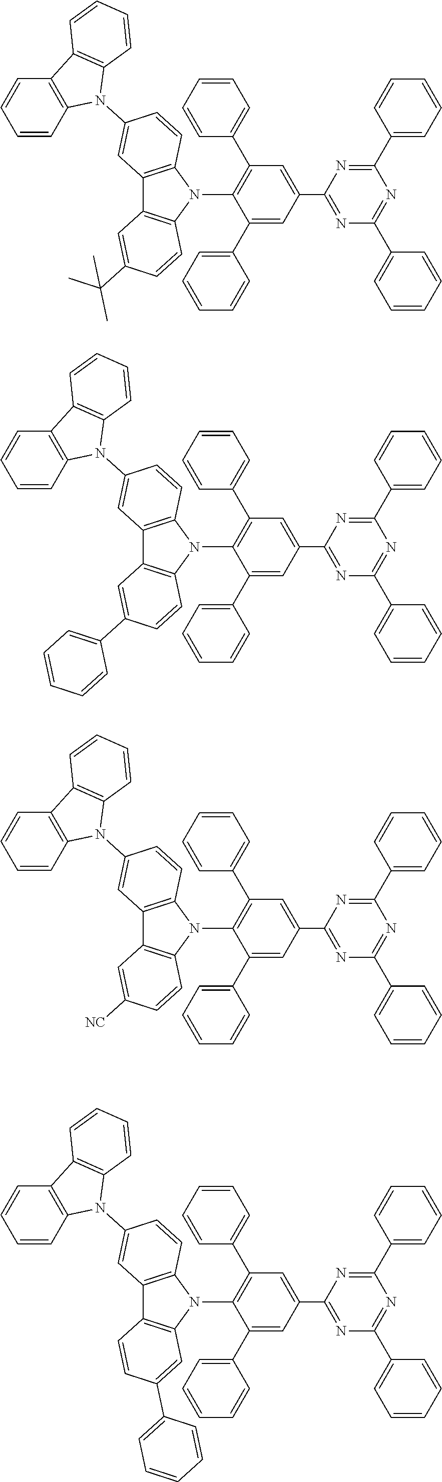

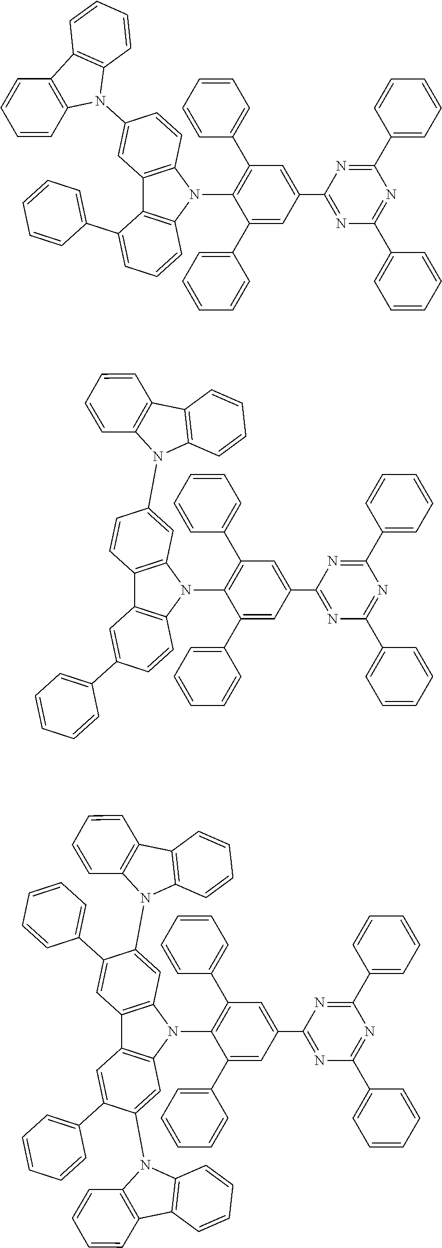 [SMILES: c1ccc(-c2nc(-c3ccccc3)nc(-c3cc(-c4ccccc4)c(-n4c5ccc(-n6c7ccccc7c7ccccc76)cc5c5c(-c6ccccc6)cccc54)c(-c4ccccc4)c3)n2)cc1 ; c1ccc(-c2ccc3c(c2)c2ccc(-n4c5ccccc5c5ccccc54)cc2n3-c2c(-c3ccccc3)cc(-c3nc(-c4ccccc4)nc(-c4ccccc4)n3)cc2-c2ccccc2)cc1 ; c1ccc(-c2nc(-c3ccccc3)nc(-c3cc(-c4ccccc4)c(-n4c5cc(-n6c7ccccc7c7ccccc76)c(-c6ccccc6)cc5c5cc(-c6ccccc6)c(-n6c7ccccc7c7ccccc76)cc54)c(-c4ccccc4)c3)n2)cc1]

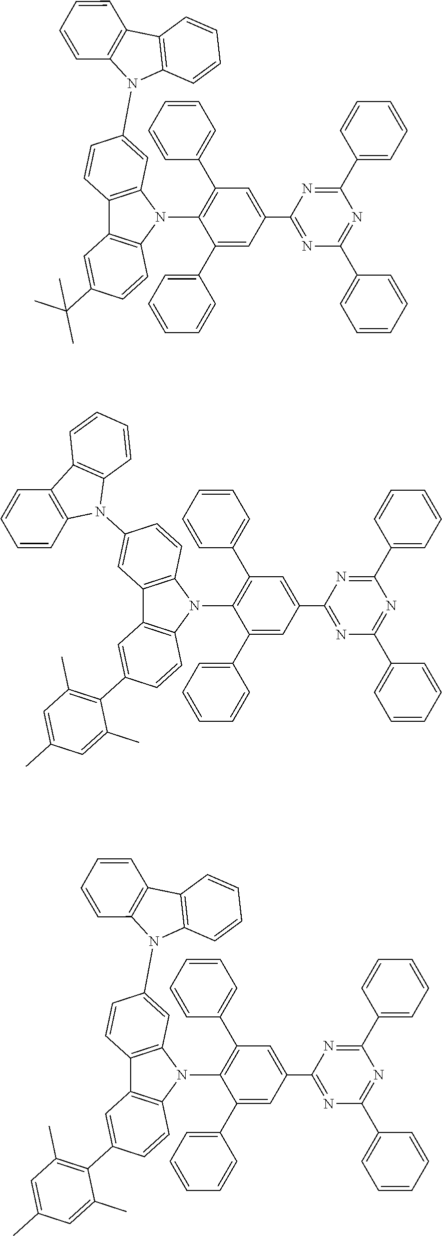 [SMILES: CC(C)(C)c1ccc2c(c1)c1ccc(-n3c4ccccc4c4ccccc43)cc1n2-c1c(-c2ccccc2)cc(-c2nc(-c3ccccc3)nc(-c3ccccc3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccccc4)nc(-c4ccccc4)n3)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccccc4)nc(-c4ccccc4)n3)cc2-c2ccccc2)c(C)c1]

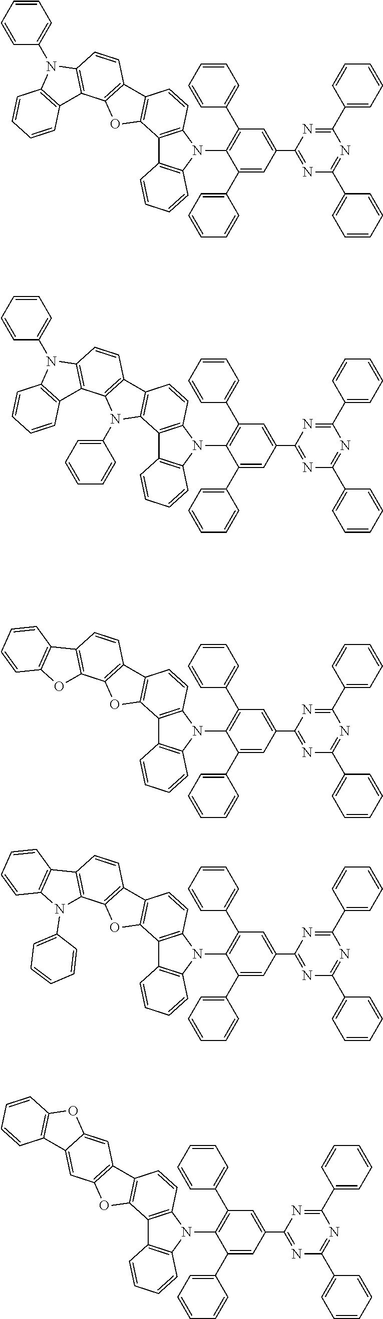

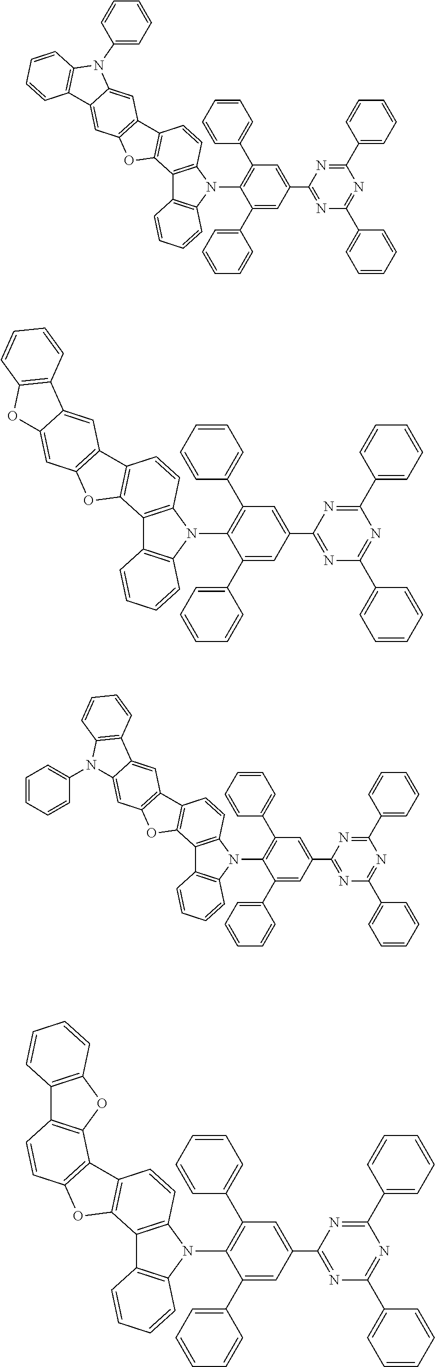 [SMILES: c1ccc(-c2nc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1]

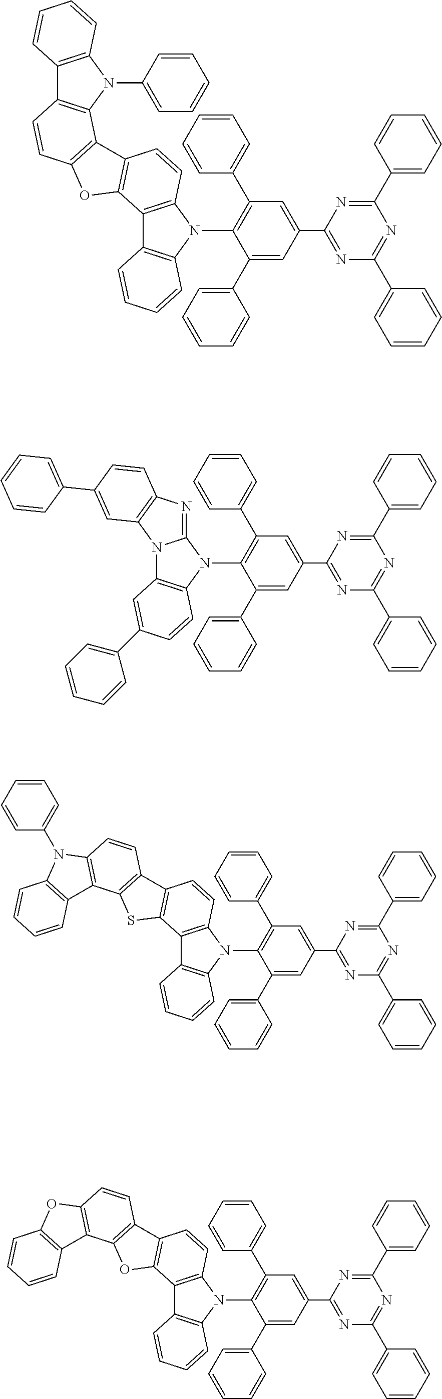 [SMILES: c1ccc(-c2nc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1 ; c1ccc(-c2ccc3nc4n(-c5c(-c6ccccc6)cc(-c6nc(-c7ccccc7)nc(-c7ccccc7)n6)cc5-c5ccccc5)c5ccc(-c6ccccc6)cc5n4c3c2)cc1 ; c1ccc(-c2nc(-c3ccccc3)nc(-c3cc(-c4ccccc4)c(-n4c5ccccc5c5c6sc7c(ccc8c7c7ccccc7n8-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1]

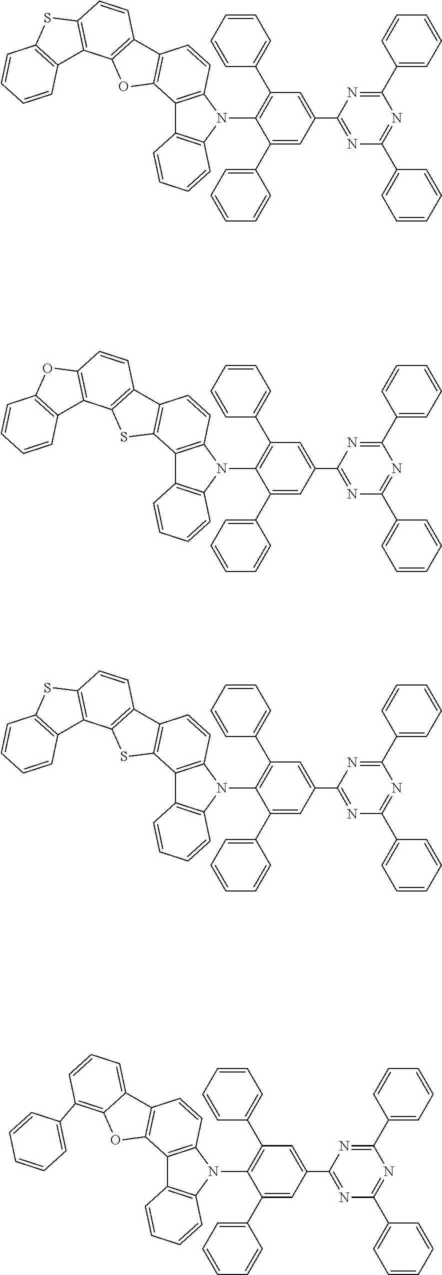 [SMILES: c1ccc(-c2nc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6sc7c(ccc8oc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6sc7c(ccc8sc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(-c8ccccc8)cccc7c6ccc54)c(-c4ccccc4)c3)n2)cc1]

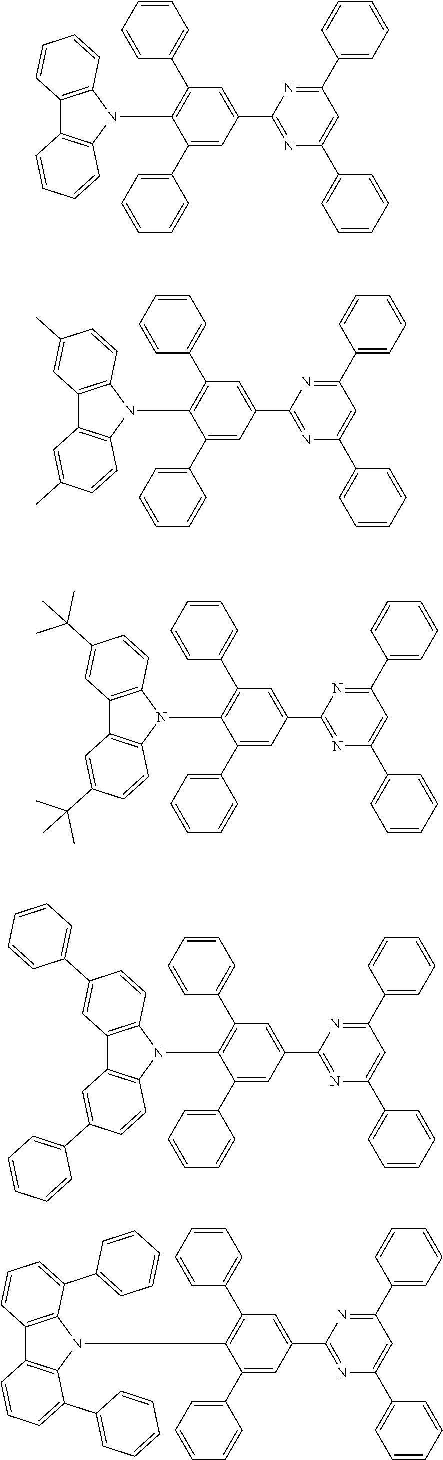 [SMILES: c1ccc(-c2cc(-c3ccccc3)nc(-c3cc(-c4ccccc4)c(-n4c5ccccc5c5ccccc54)c(-c4ccccc4)c3)n2)cc1 ; Cc1ccc2c(c1)c1cc(C)ccc1n2-c1c(-c2ccccc2)cc(-c2nc(-c3ccccc3)cc(-c3ccccc3)n2)cc1-c1ccccc1 ; CC(C)(C)c1ccc2c(c1)c1cc(C(C)(C)C)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)cc(-c4ccccc4)n3)cc2-c2ccccc2)cc1 ; c1ccc(-c2cc(-c3ccccc3)nc(-c3cc(-c4ccccc4)c(-n4c5c(-c6ccccc6)cccc5c5cccc(-c6ccccc6)c54)c(-c4ccccc4)c3)n2)cc1]

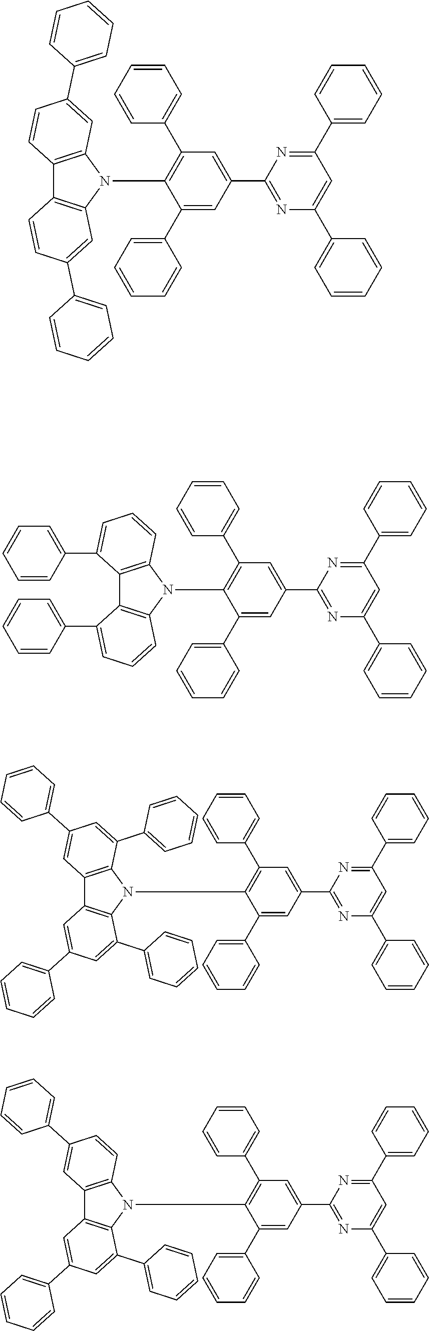 [SMILES: c1ccc(-c2ccc3c4ccc(-c5ccccc5)cc4n(-c4c(-c5ccccc5)cc(-c5nc(-c6ccccc6)cc(-c6ccccc6)n5)cc4-c4ccccc4)c3c2)cc1 ; c1ccc(-c2cc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1]

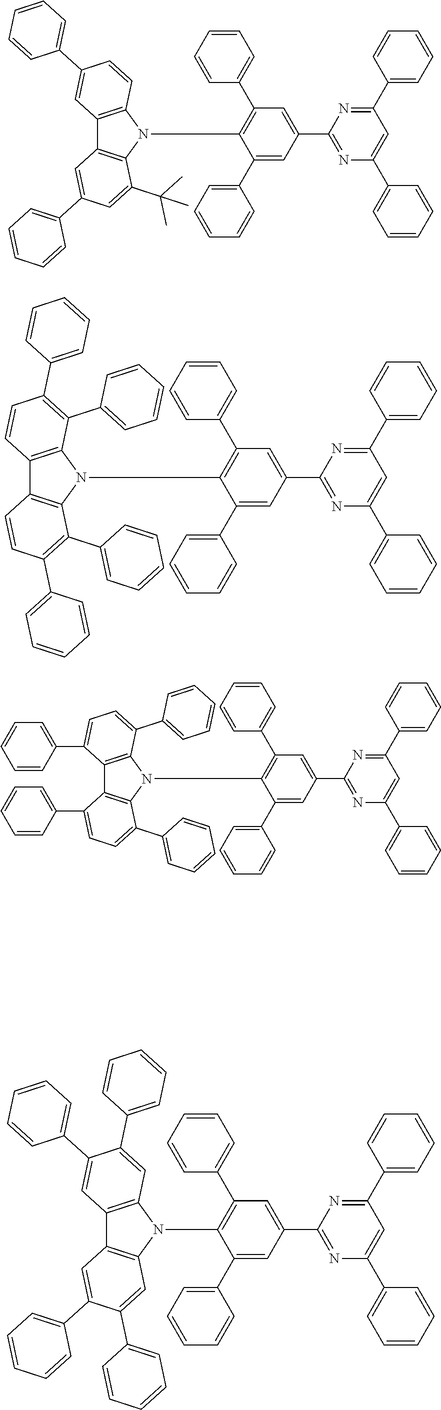

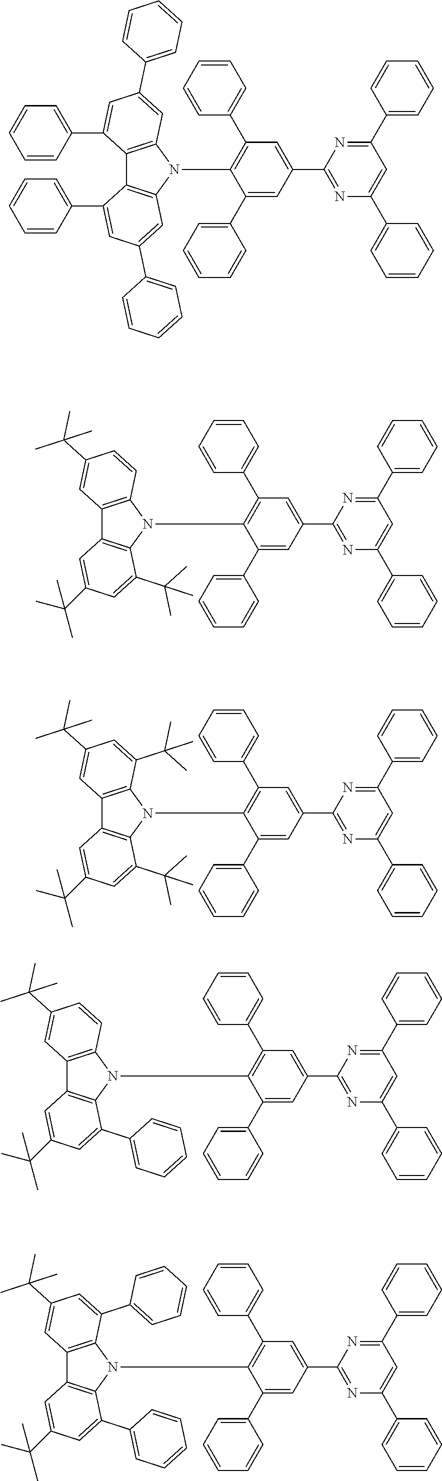

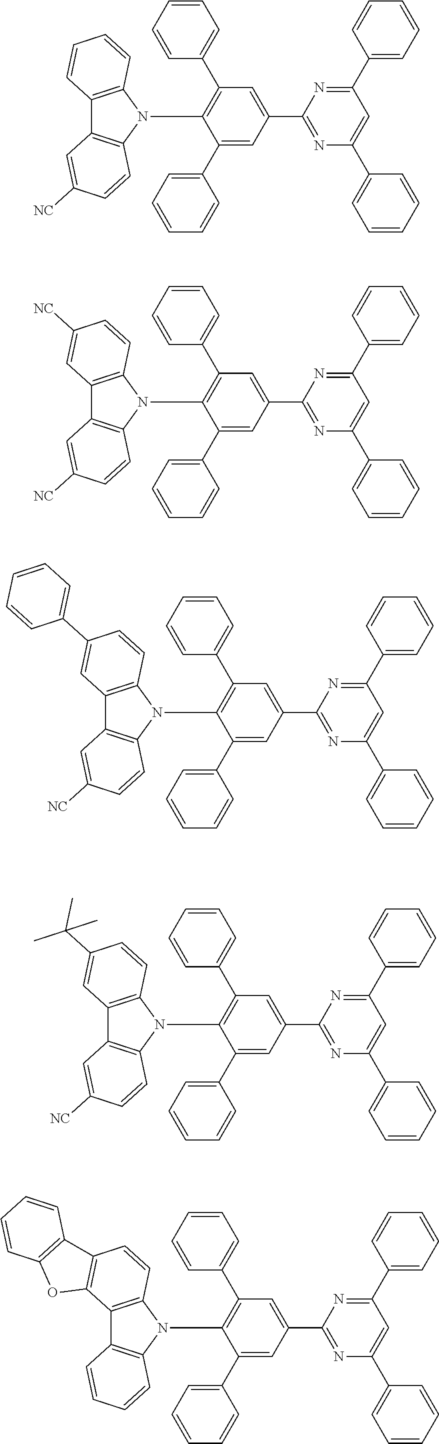 [SMILES: N#Cc1ccc2c(c1)c1ccccc1n2-c1c(-c2ccccc2)cc(-c2nc(-c3ccccc3)cc(-c3ccccc3)n2)cc1-c1ccccc1 ; N#Cc1ccc2c(c1)c1cc(C#N)ccc1n2-c1c(-c2ccccc2)cc(-c2nc(-c3ccccc3)cc(-c3ccccc3)n2)cc1-c1ccccc1 ; N#Cc1ccc2c(c1)c1cc(-c3ccccc3)ccc1n2-c1c(-c2ccccc2)cc(-c2nc(-c3ccccc3)cc(-c3ccccc3)n2)cc1-c1ccccc1 ; CC(C)(C)c1ccc2c(c1)c1cc(C#N)ccc1n2-c1c(-c2ccccc2)cc(-c2nc(-c3ccccc3)cc(-c3ccccc3)n2)cc1-c1ccccc1 ; c1ccc(-c2cc(-c3ccccc3)nc(-c3cc(-c4ccccc4)c(-n4c5ccccc5c5c6oc7ccccc7c6ccc54)c(-c4ccccc4)c3)n2)cc1]

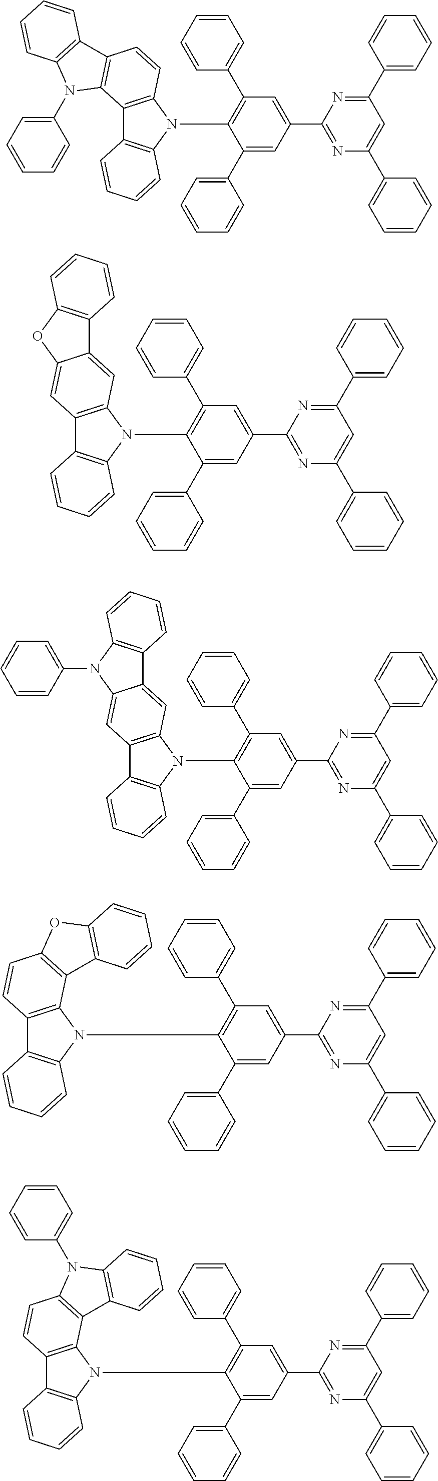

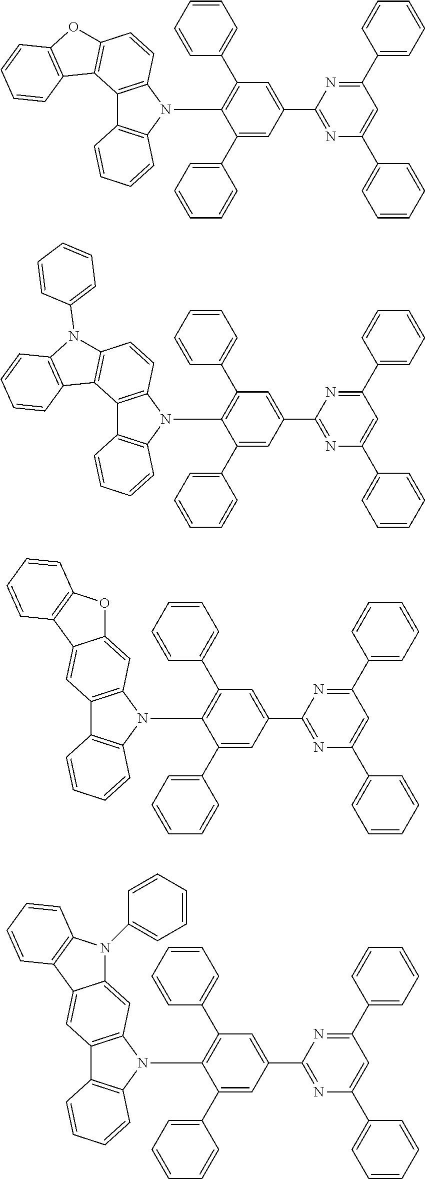

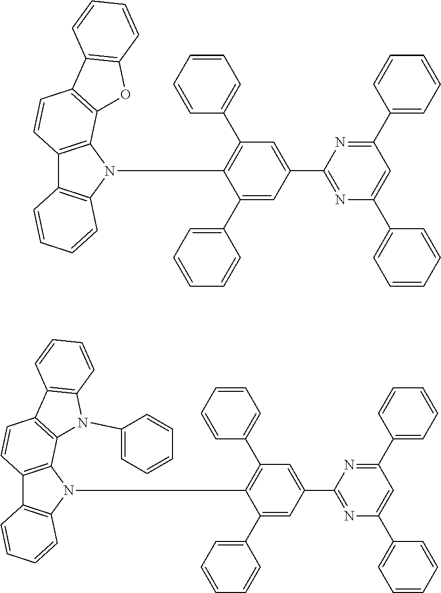 [SMILES: c1ccc(-c2cc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2cc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1]

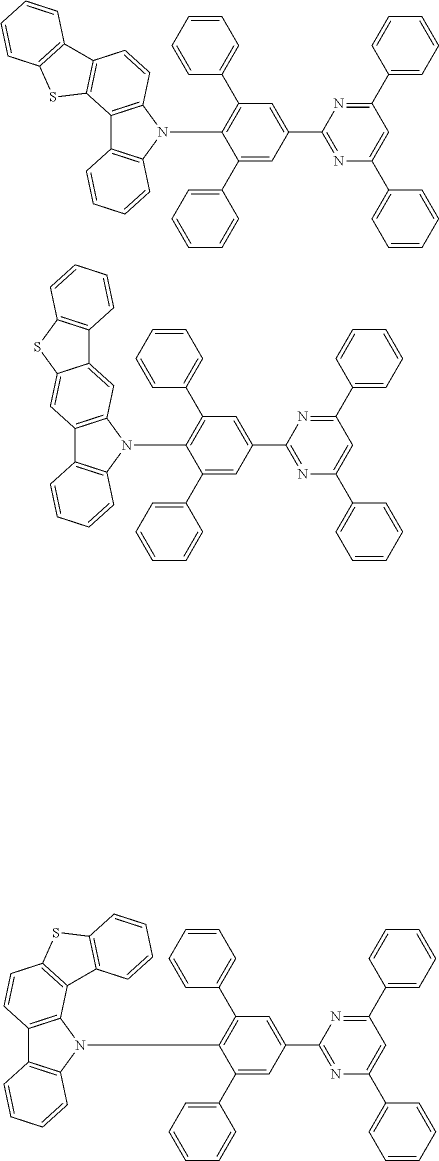 [SMILES: c1ccc(-c2cc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2cc(-c3ccccc3)nc(-c3cc(-c4ccccc4)c(-n4c5ccccc5c5cc6sc7ccccc7c6cc54)c(-c4ccccc4)c3)n2)cc1 ; c1ccc(-c2cc(-c3ccccc3)nc(-c3cc(-c4ccccc4)c(-n4c5ccccc5c5ccc6sc7ccccc7c6c54)c(-c4ccccc4)c3)n2)cc1]

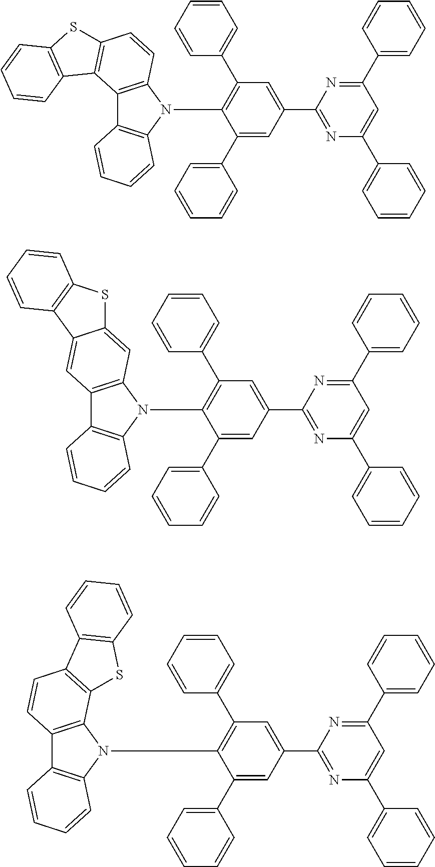 [SMILES: c1ccc(-c2cc(-c3ccccc3)nc(-c3cc(-c4ccccc4)c(-n4c5ccccc5c5c6c(ccc54)sc4ccccc46)c(-c4ccccc4)c3)n2)cc1 ; c1ccc(-c2cc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2cc(-c3ccccc3)nc(-c3cc(-c4ccccc4)c(-n4c5ccccc5c5ccc6c7ccccc7sc6c54)c(-c4ccccc4)c3)n2)cc1]

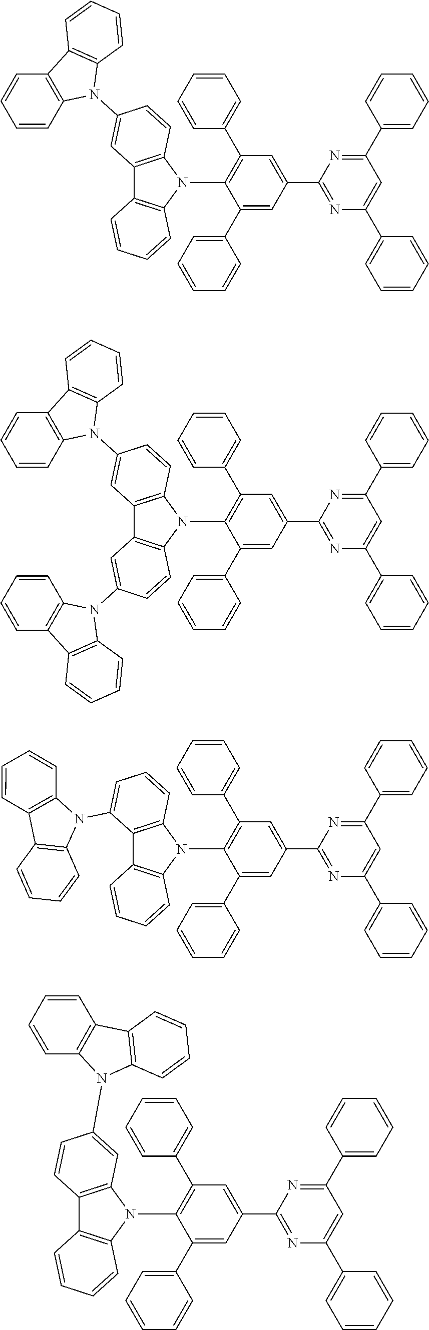 [SMILES: c1ccc(-c2cc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2cc(-c3ccccc3)nc(-c3cc(-c4ccccc4)c(-n4c5ccccc5c5ccc(-n6c7ccccc7c7ccccc76)cc54)c(-c4ccccc4)c3)n2)cc1]

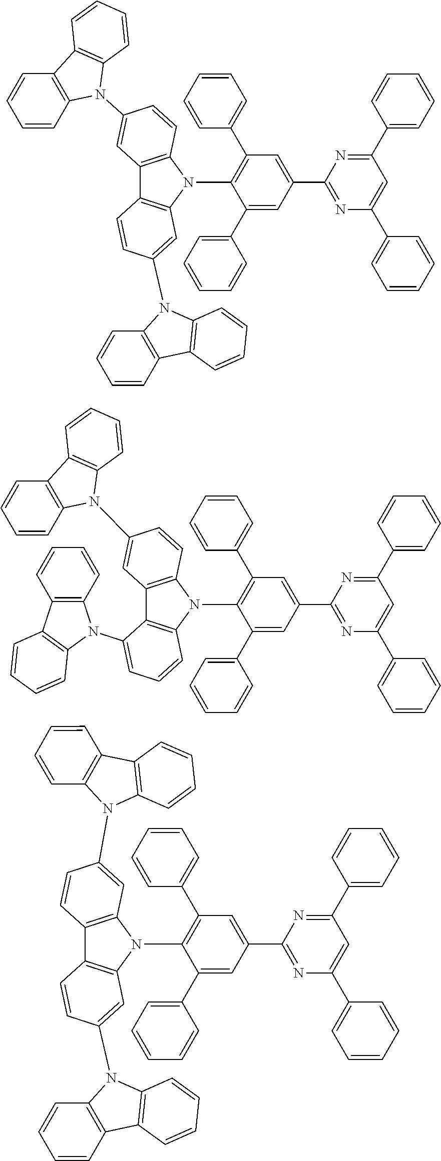

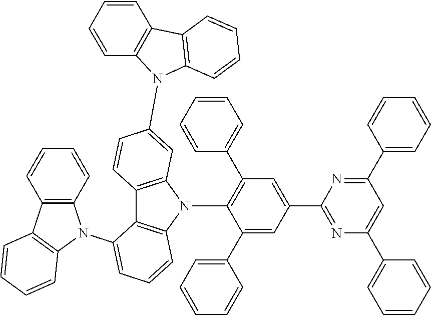 [SMILES: c1ccc(-c2cc(-c3ccccc3)nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)n2)cc1]

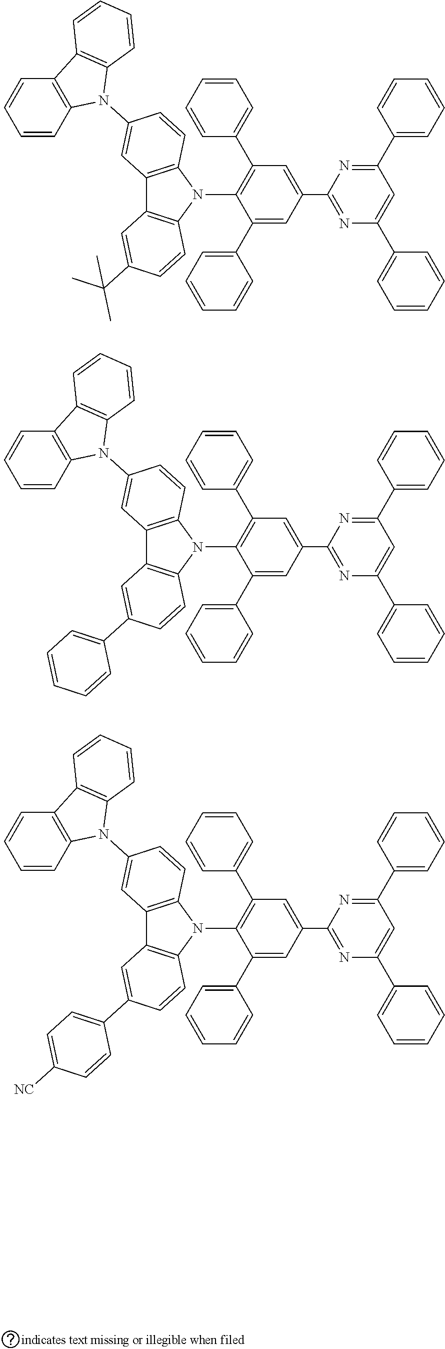

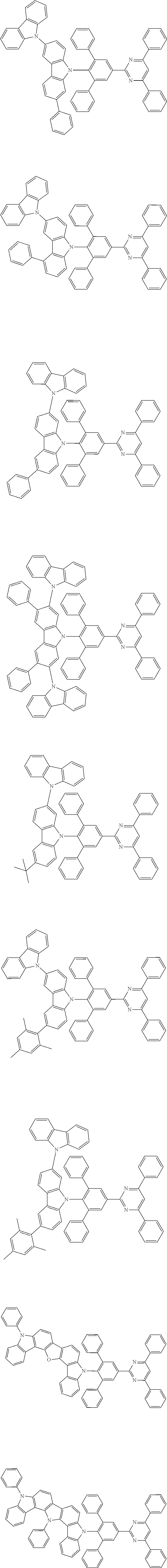

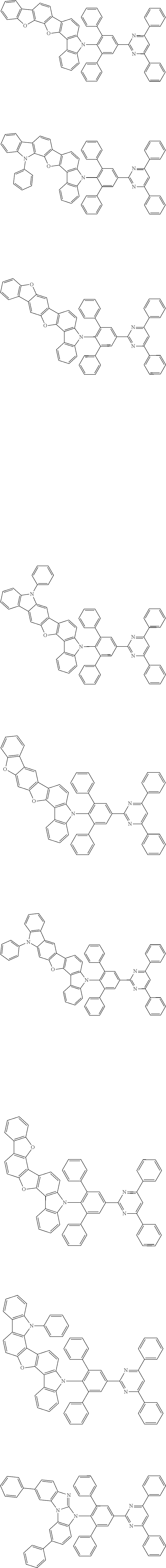

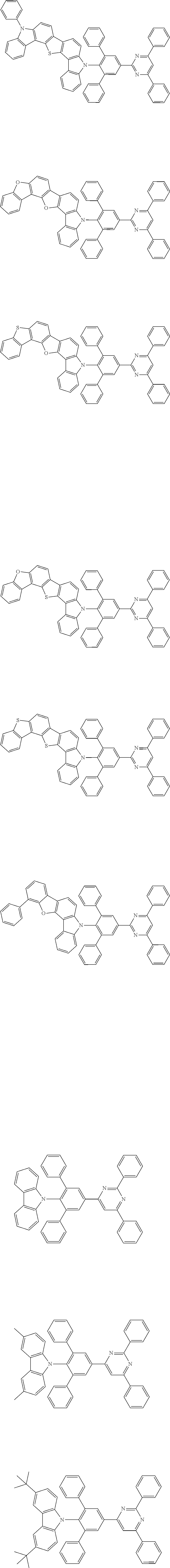

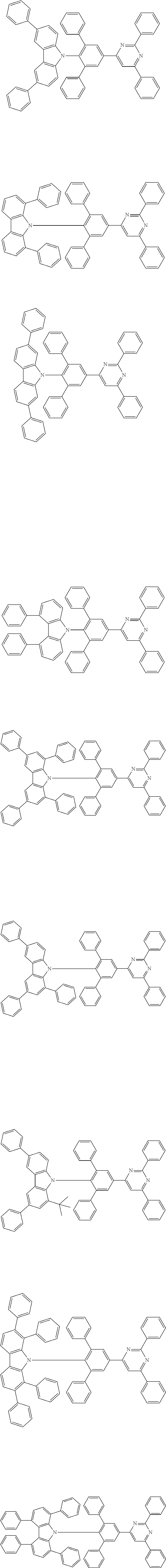

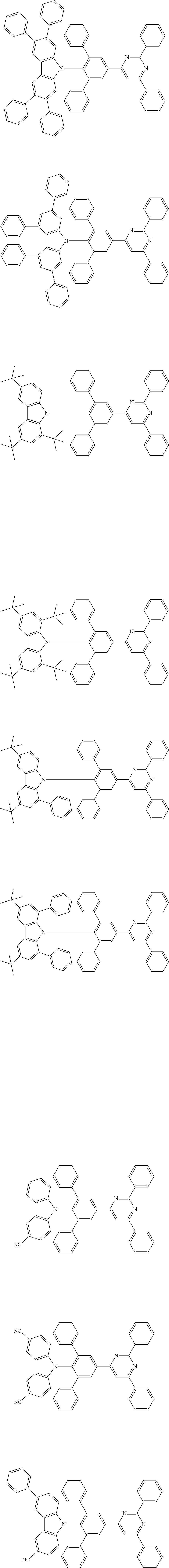

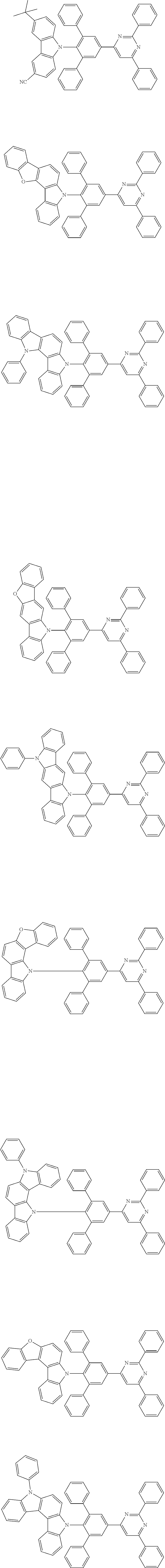

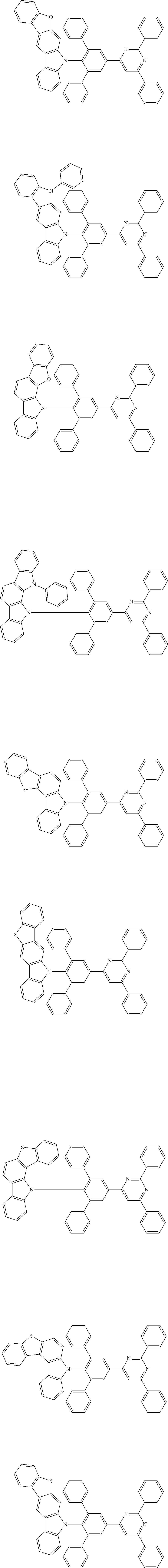

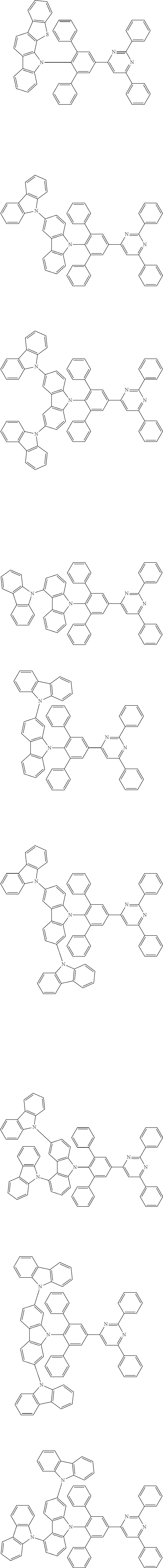

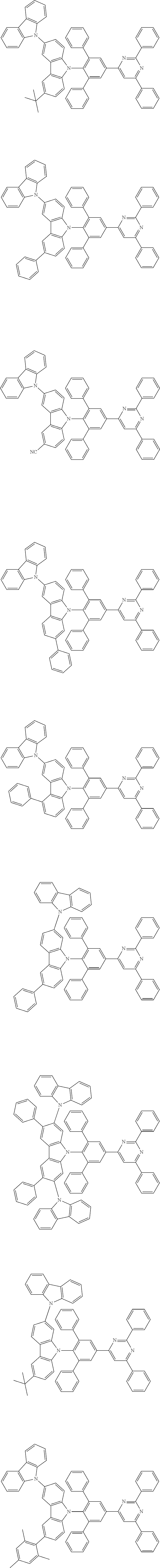

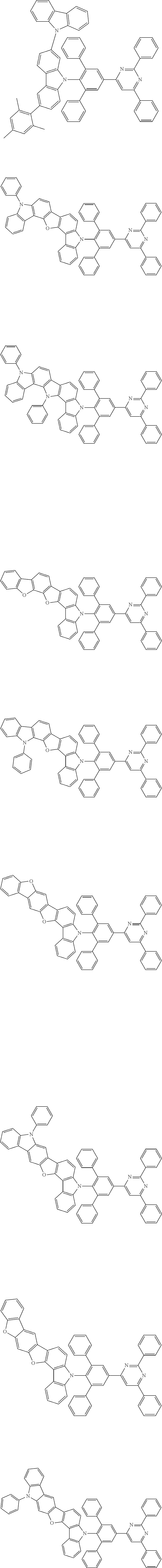

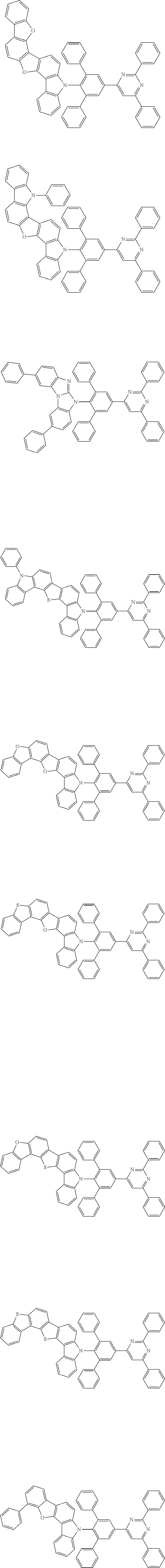

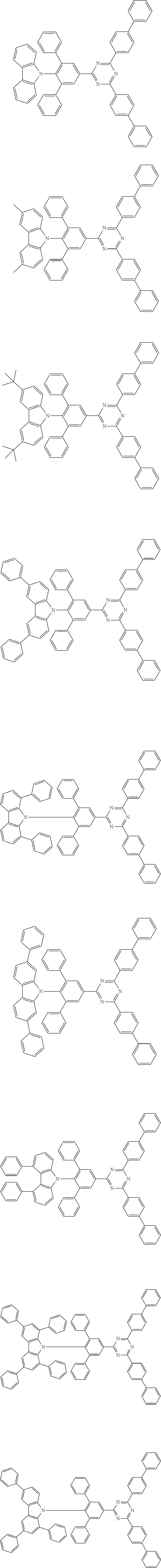

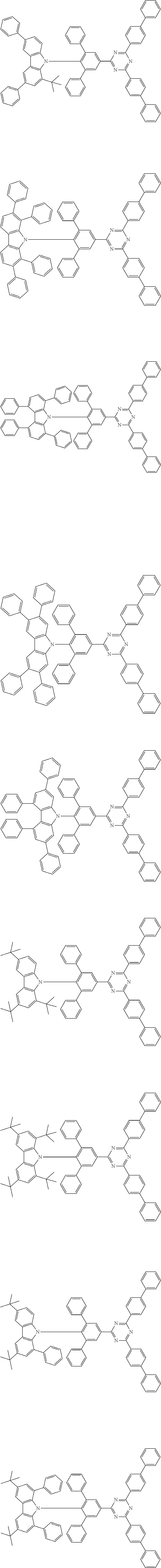

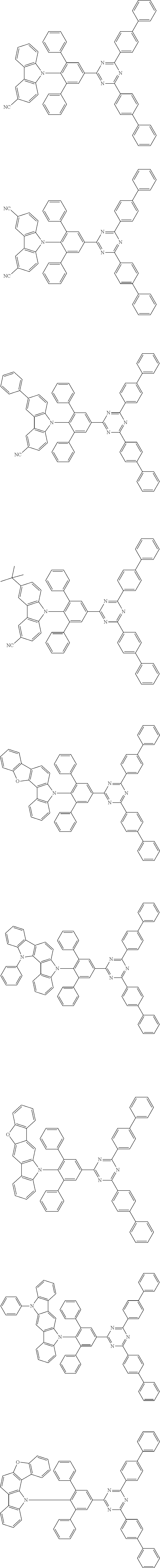

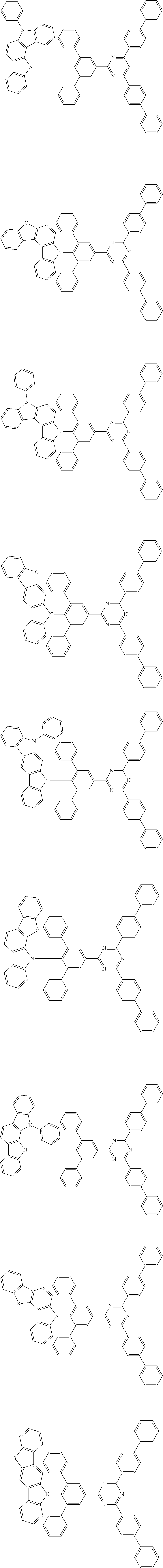

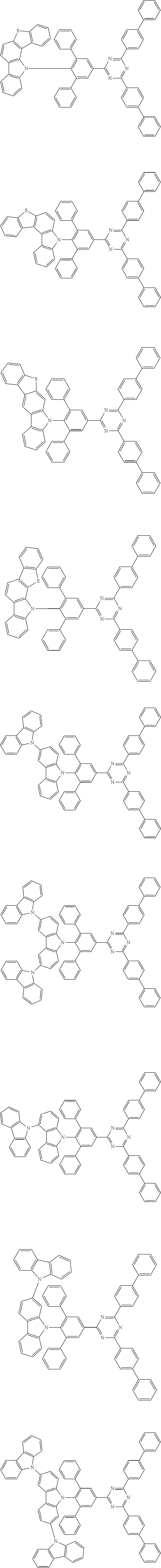

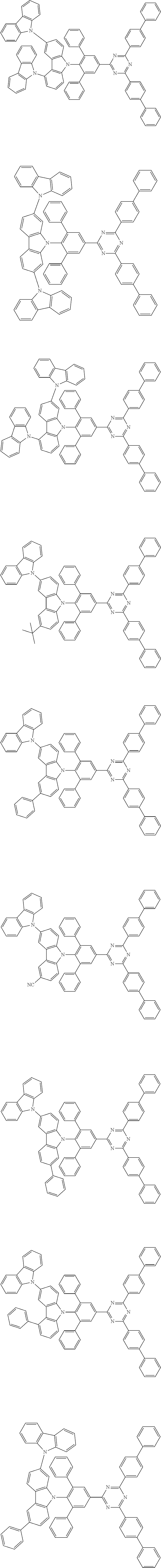

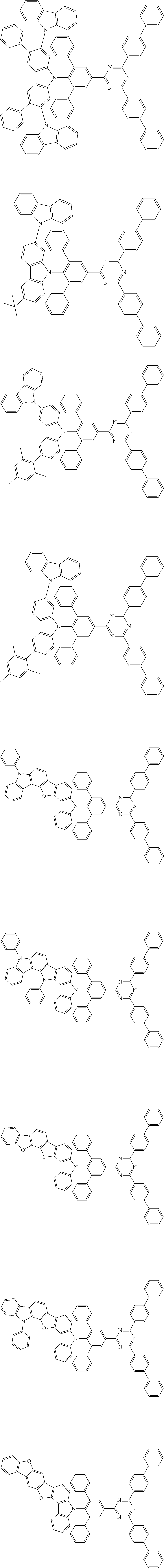

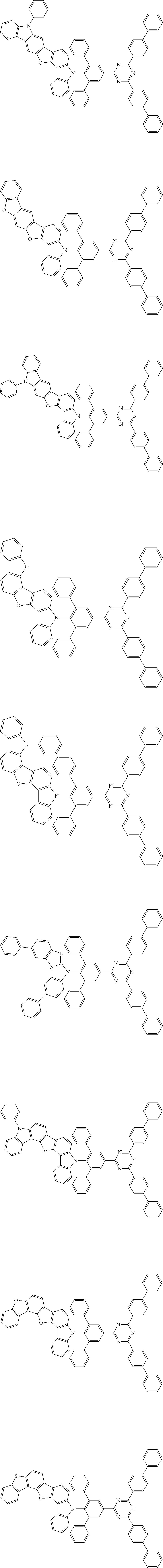

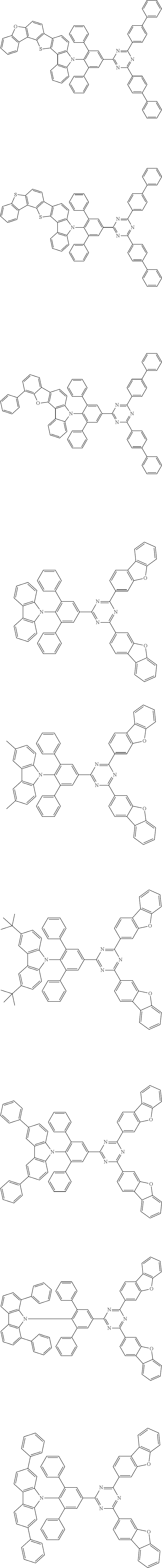

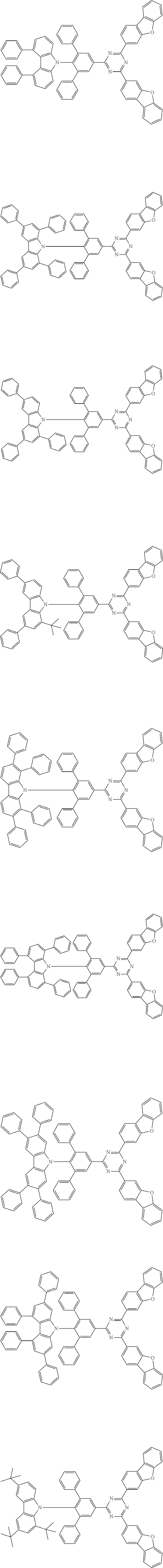

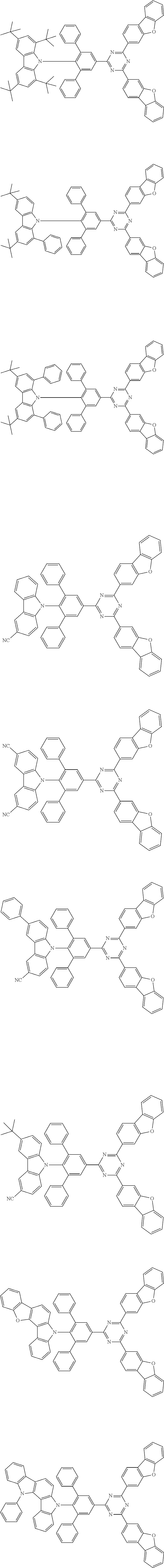

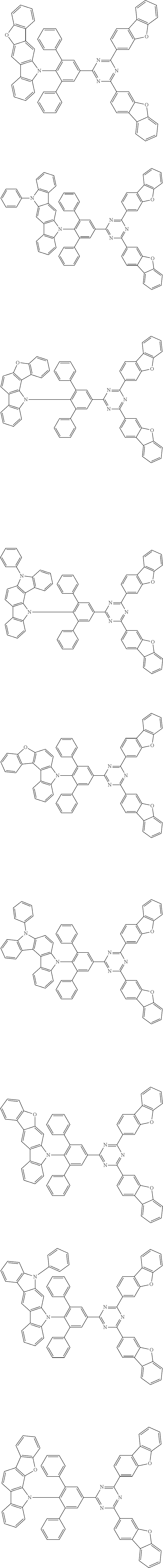

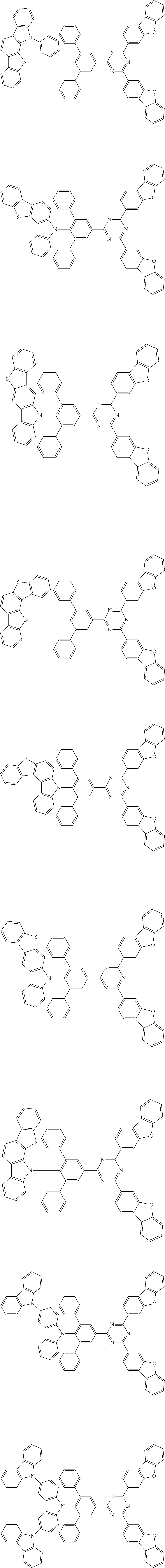

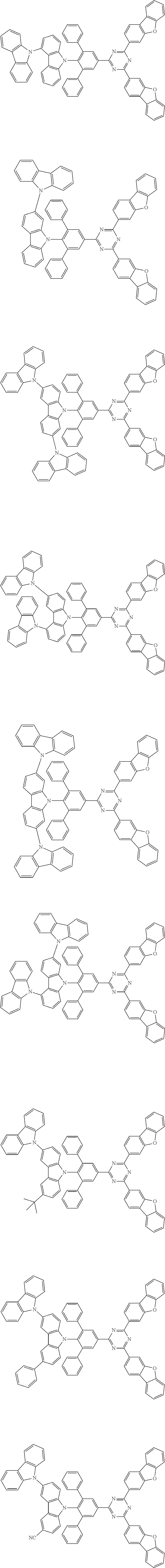

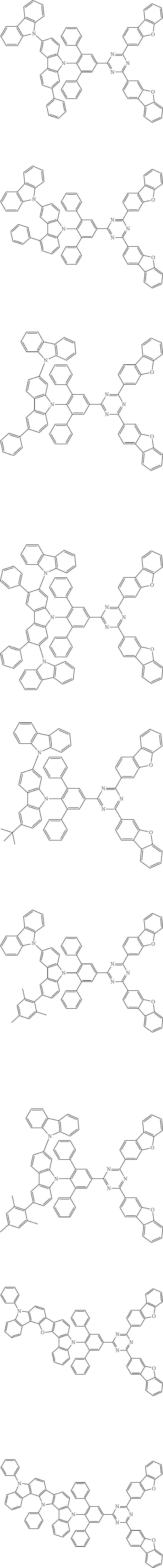

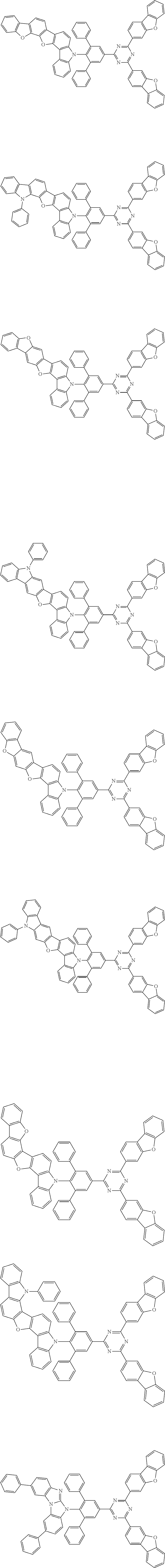

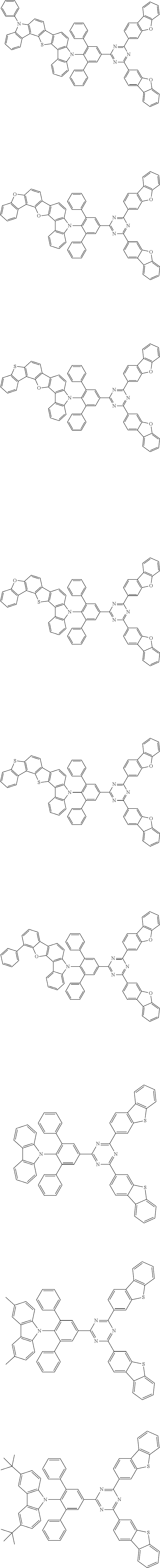

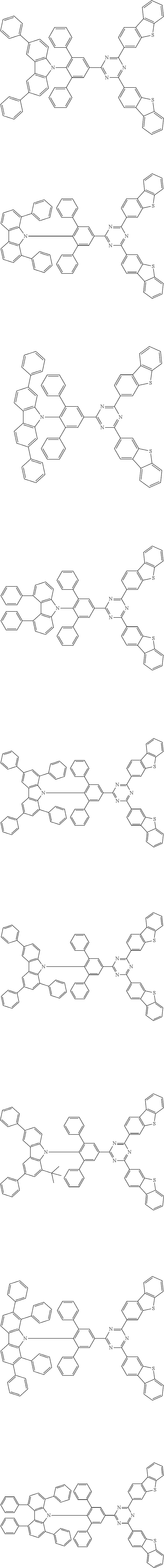

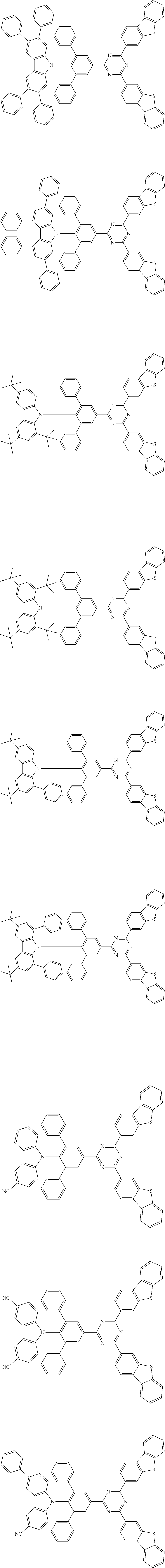

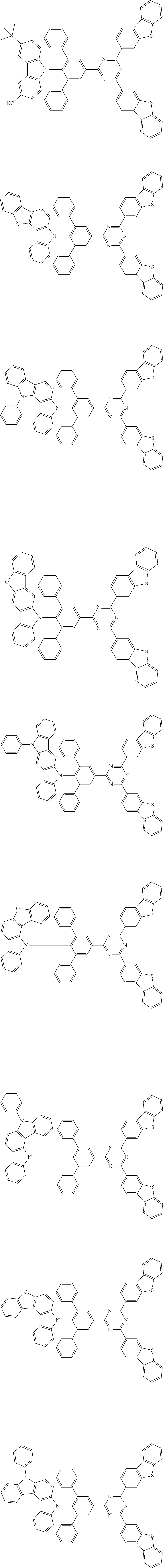

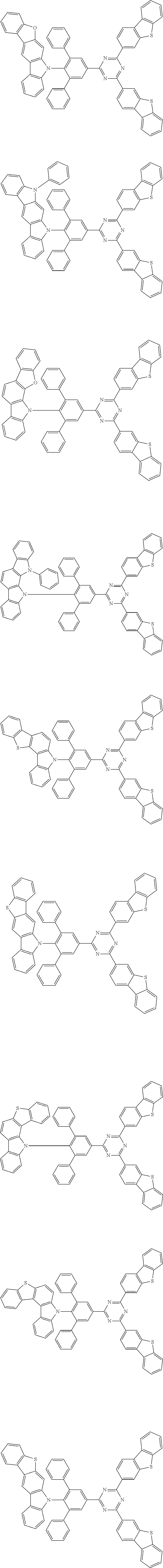

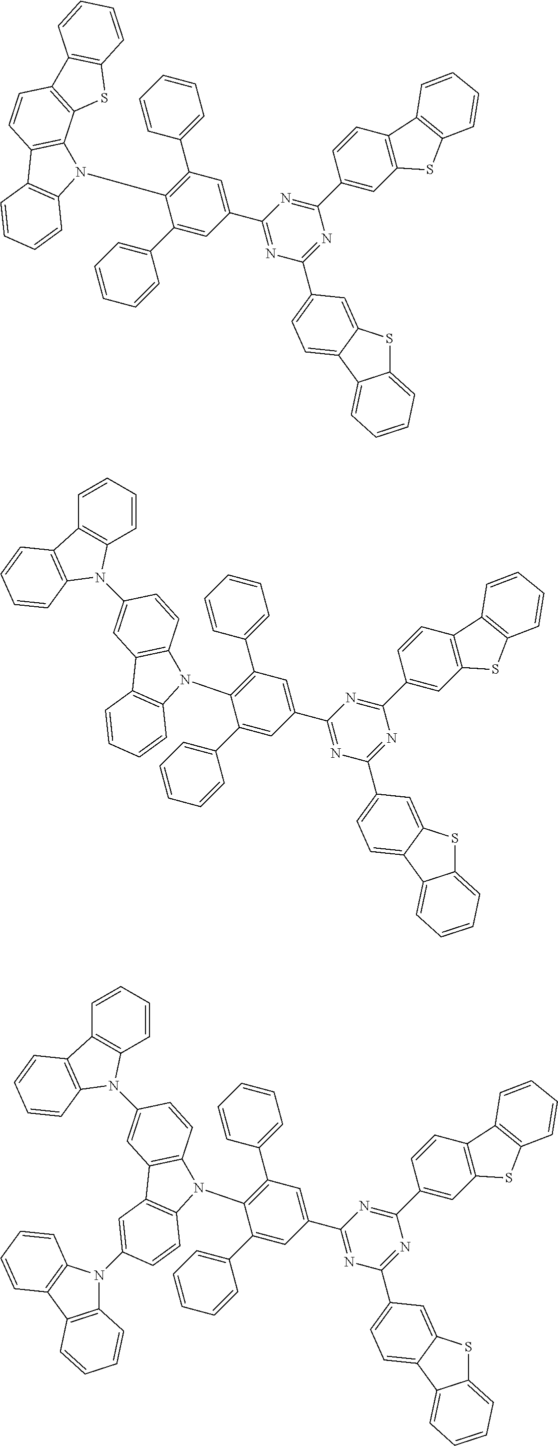 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc4c5ccccc5sc4c32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3cc(-n4c5ccccc5c5ccccc54)ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3cc(-n4c5ccccc5c5ccccc54)ccc32)cc1]

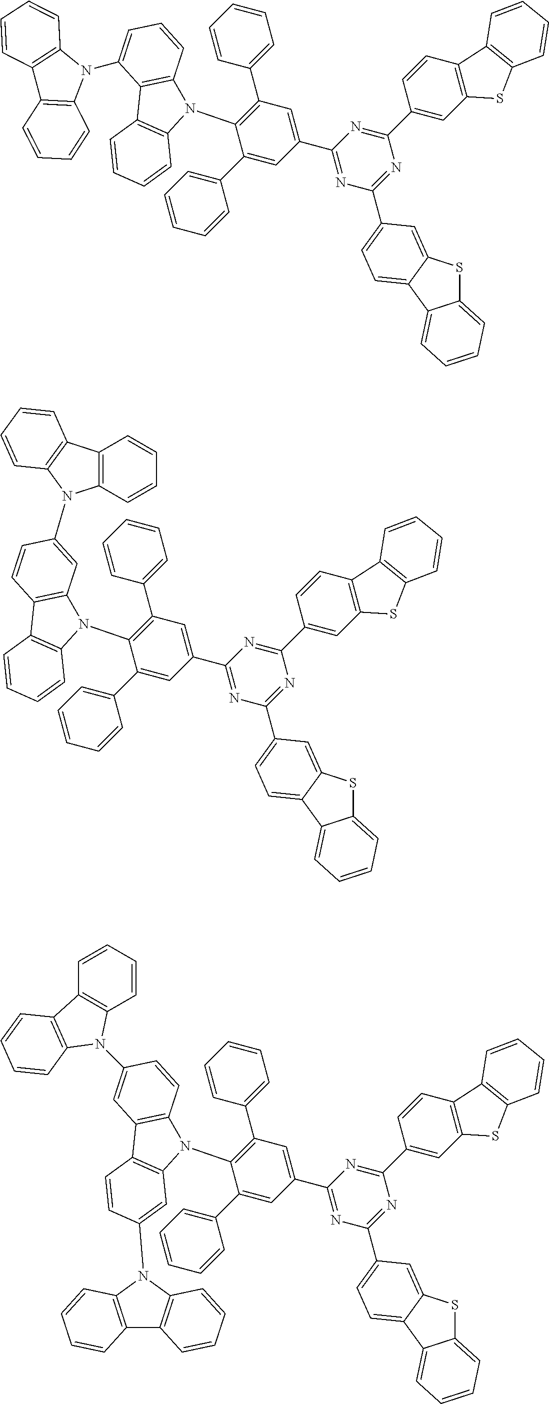 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c(-n4c5ccccc5c5ccccc54)cccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3ccc(-n4c5ccccc5c5ccccc54)cc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3ccc(-n4c5ccccc5c5ccccc54)cc32)cc1]

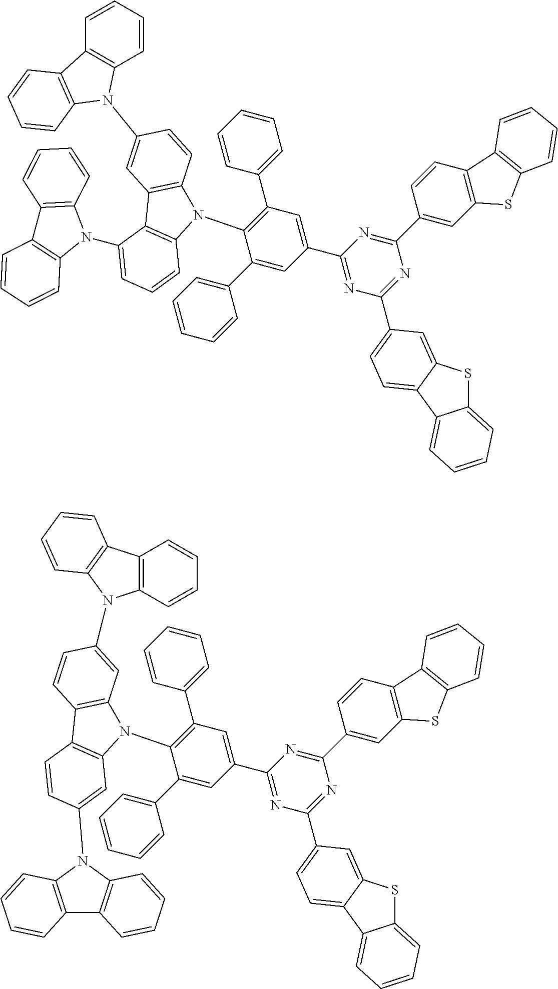 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3c(-n4c5ccccc5c5ccccc54)cccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3cc(-n4c5ccccc5c5ccccc54)ccc3c3ccc(-n4c5ccccc5c5ccccc54)cc32)cc1]

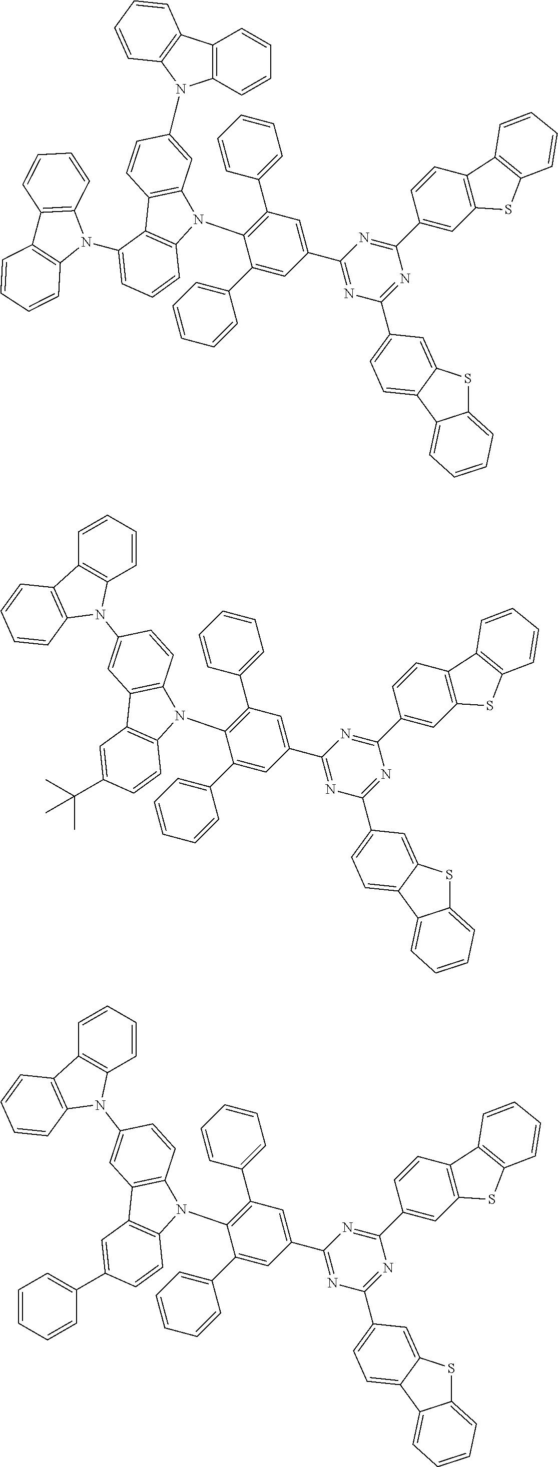 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3cc(-n4c5ccccc5c5ccccc54)ccc3c3c(-n4c5ccccc5c5ccccc54)cccc32)cc1 ; CC(C)(C)c1ccc2c(c1)c1cc(-n3c4ccccc4c4ccccc43)ccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccc5c(c4)sc4ccccc45)nc(-c4ccc5c(c4)sc4ccccc45)n3)cc2-c2ccccc2)cc1]

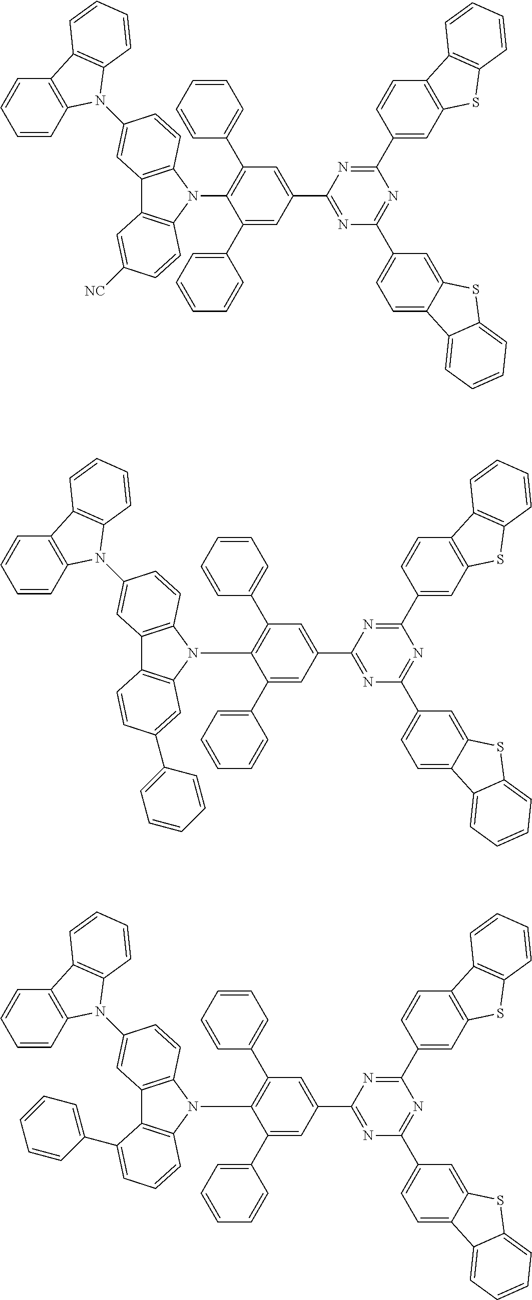 [SMILES: N#Cc1ccc2c(c1)c1cc(-n3c4ccccc4c4ccccc43)ccc1n2-c1c(-c2ccccc2)cc(-c2nc(-c3ccc4c(c3)sc3ccccc34)nc(-c3ccc4c(c3)sc3ccccc34)n2)cc1-c1ccccc1 ; c1ccc(-c2ccc3c4cc(-n5c6ccccc6c6ccccc65)ccc4n(-c4c(-c5ccccc5)cc(-c5nc(-c6ccc7c(c6)sc6ccccc67)nc(-c6ccc7c(c6)sc6ccccc67)n5)cc4-c4ccccc4)c3c2)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-n4c5ccccc5c5ccccc54)cc3c3c(-c4ccccc4)cccc32)cc1]

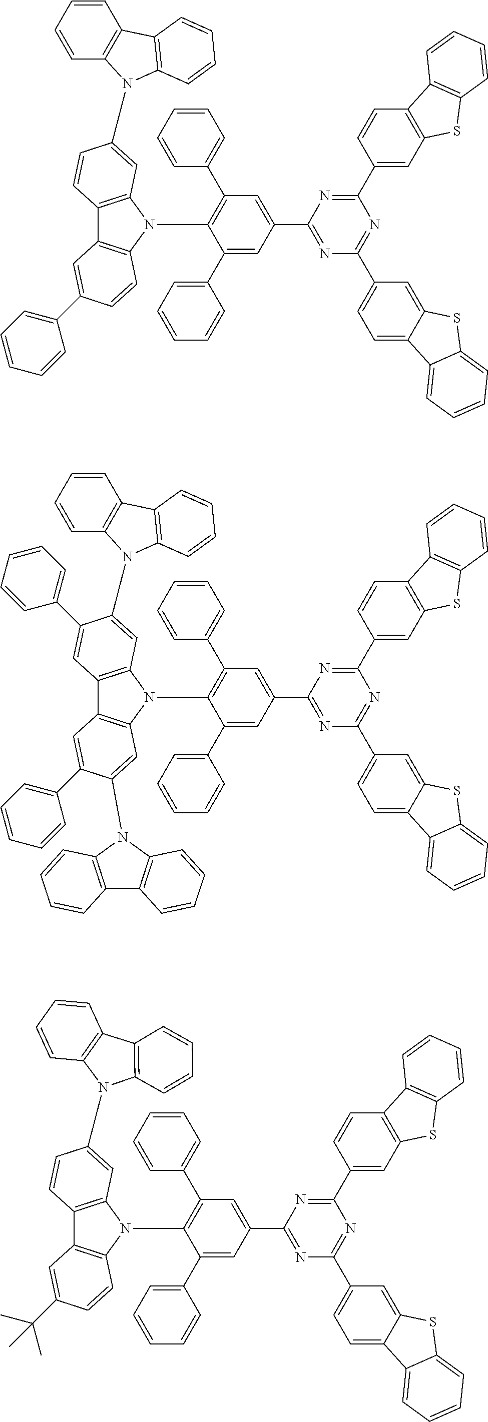 [SMILES: c1ccc(-c2ccc3c(c2)c2ccc(-n4c5ccccc5c5ccccc54)cc2n3-c2c(-c3ccccc3)cc(-c3nc(-c4ccc5c(c4)sc4ccccc45)nc(-c4ccc5c(c4)sc4ccccc45)n3)cc2-c2ccccc2)cc1 ; c1ccc(-c2cc3c4cc(-c5ccccc5)c(-n5c6ccccc6c6ccccc65)cc4n(-c4c(-c5ccccc5)cc(-c5nc(-c6ccc7c(c6)sc6ccccc67)nc(-c6ccc7c(c6)sc6ccccc67)n5)cc4-c4ccccc4)c3cc2-n2c3ccccc3c3ccccc32)cc1 ; CC(C)(C)c1ccc2c(c1)c1ccc(-n3c4ccccc4c4ccccc43)cc1n2-c1c(-c2ccccc2)cc(-c2nc(-c3ccc4c(c3)sc3ccccc34)nc(-c3ccc4c(c3)sc3ccccc34)n2)cc1-c1ccccc1]

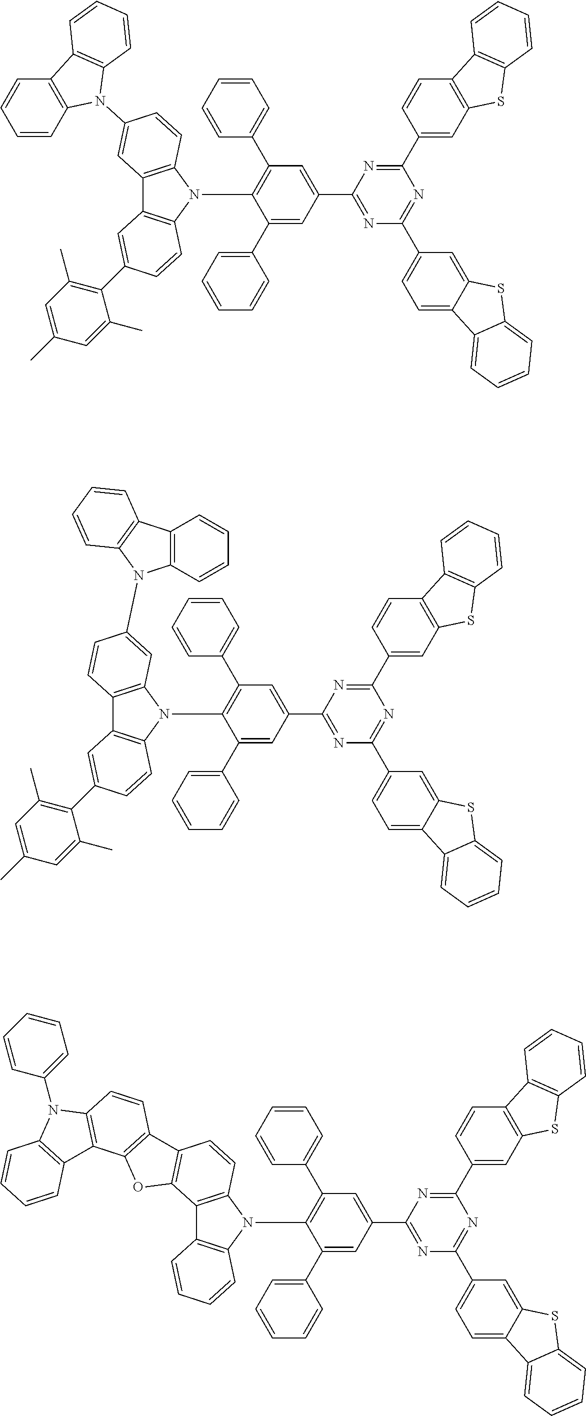 [SMILES: Cc1cc(C)c(-c2ccc3c(c2)c2cc(-n4c5ccccc5c5ccccc54)ccc2n3-c2c(-c3ccccc3)cc(-c3nc(-c4ccc5c(c4)sc4ccccc45)nc(-c4ccc5c(c4)sc4ccccc45)n3)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccc5c(c4)sc4ccccc45)nc(-c4ccc5c(c4)sc4ccccc45)n3)cc2-c2ccccc2)c(C)c1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c5c5ccccc5n6-c5ccccc5)c4ccc32)cc1]

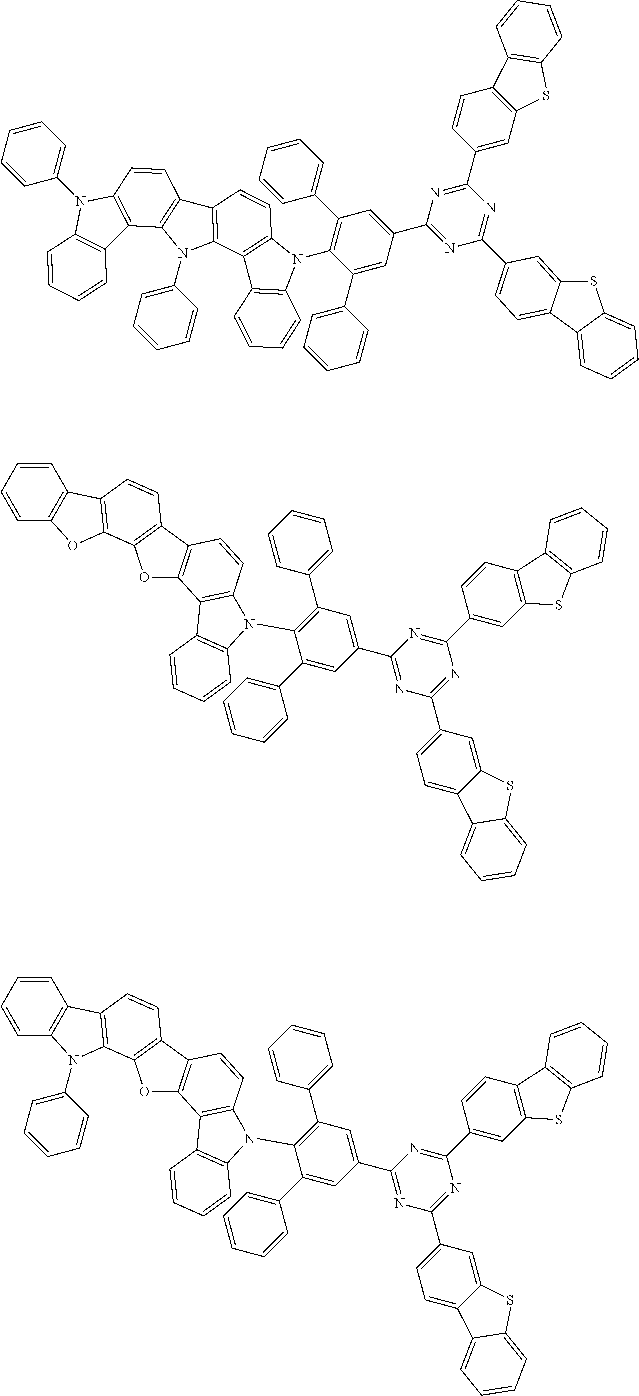 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c2ccc2c4ccc5c(c6ccccc6n5-c5ccccc5)c4n(-c4ccccc4)c23)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c7ccccc7oc65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c7ccccc7n(-c7ccccc7)c65)c4ccc32)cc1]

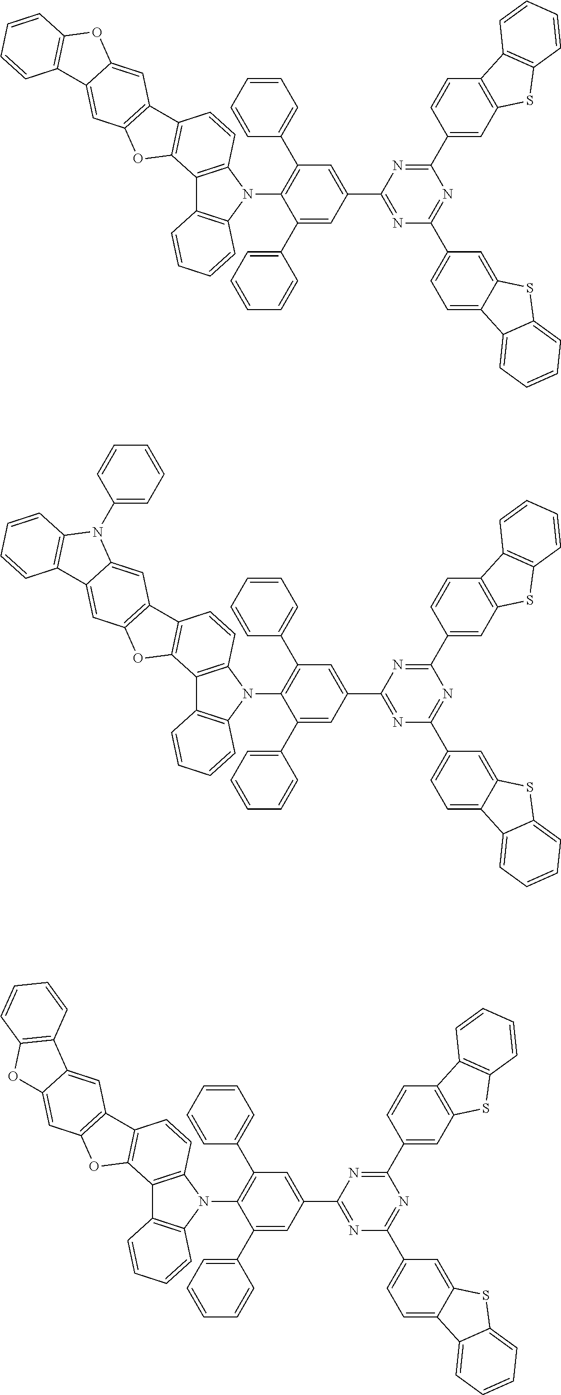 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c(cc5c4ccc32)oc2ccccc26)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c7ccccc7n(-c7ccccc7)c6cc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6oc7ccccc7c6cc5c4ccc32)cc1]

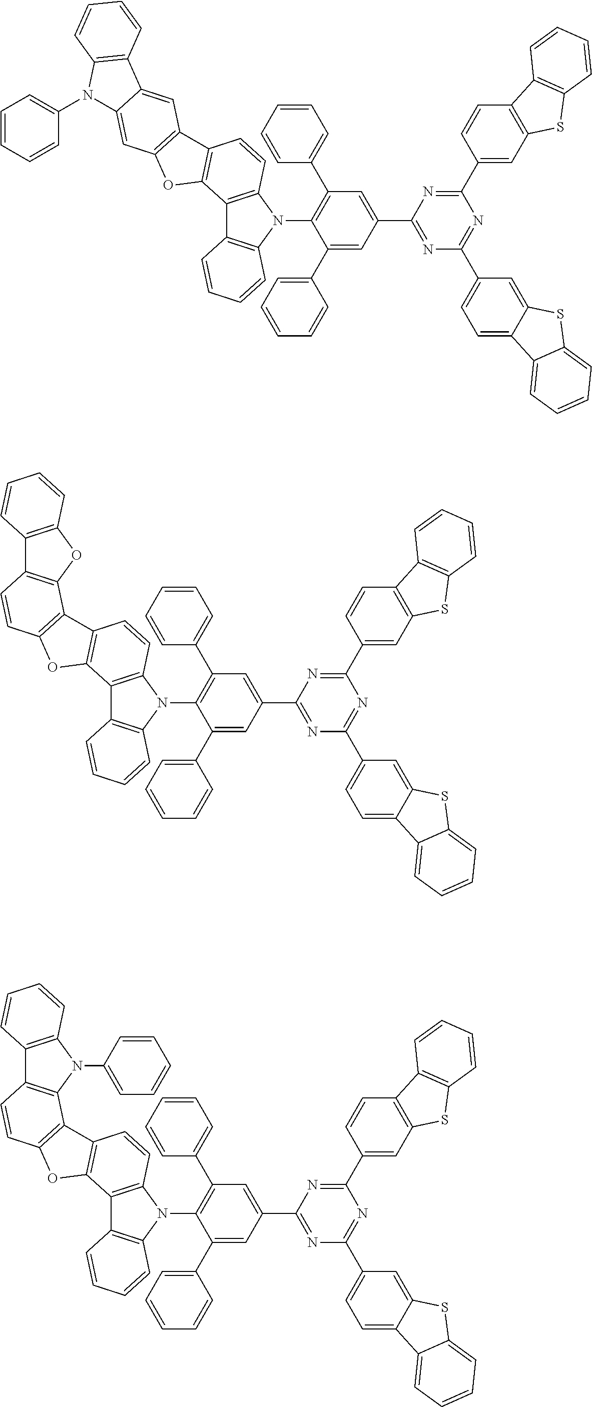 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c(cc5c4ccc32)c2ccccc2n6-c2ccccc2)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5ccc6c7ccccc7oc6c5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5ccc6c7ccccc7n(-c7ccccc7)c6c5c4ccc32)cc1]

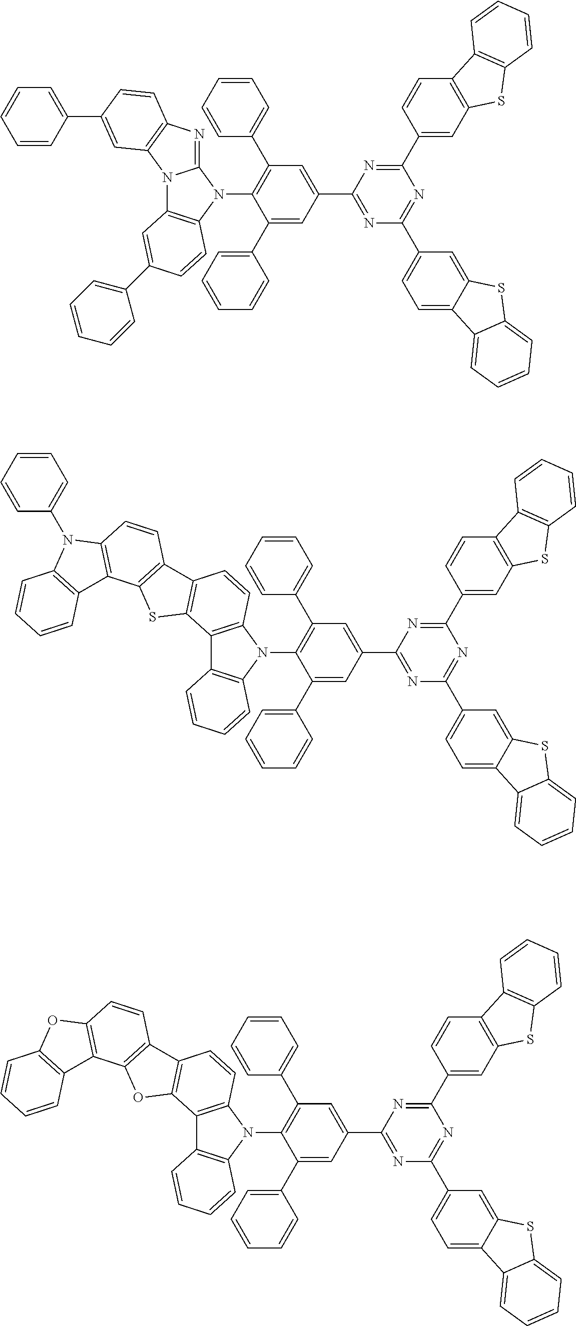 [SMILES: c1ccc(-c2ccc3nc4n(-c5c(-c6ccccc6)cc(-c6nc(-c7ccc8c(c7)sc7ccccc78)nc(-c7ccc8c(c7)sc7ccccc78)n6)cc5-c5ccccc5)c5ccc(-c6ccccc6)cc5n4c3c2)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6c5c5ccccc5n6-c5ccccc5)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6oc7ccccc7c65)c4ccc32)cc1]

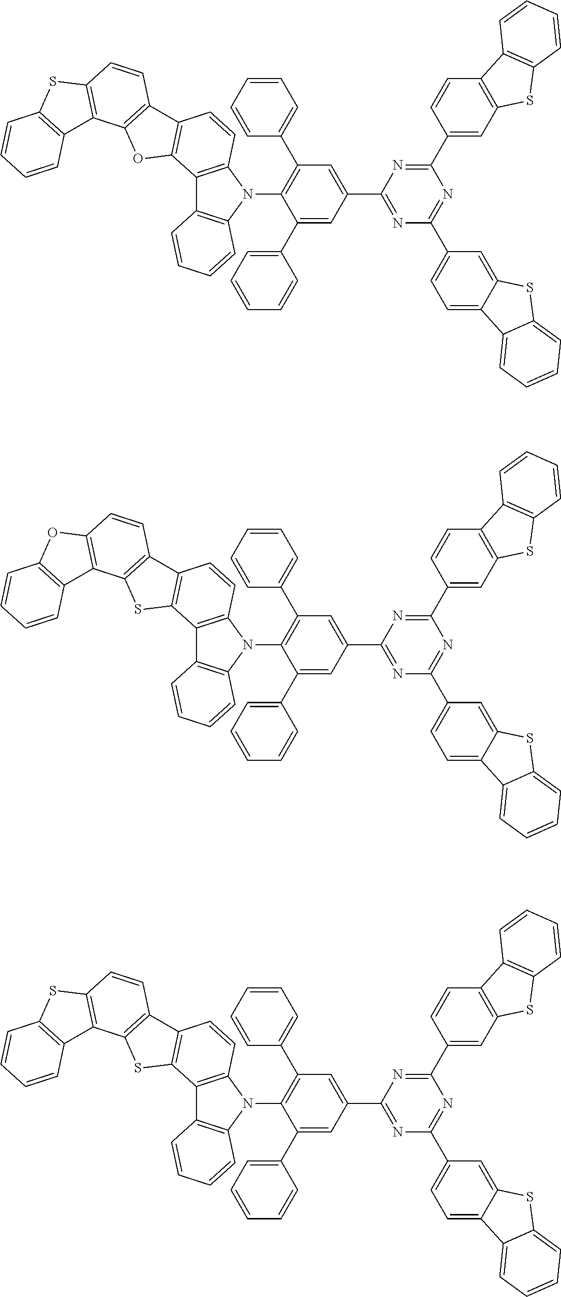 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6sc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6oc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6sc7ccccc7c65)c4ccc32)cc1]

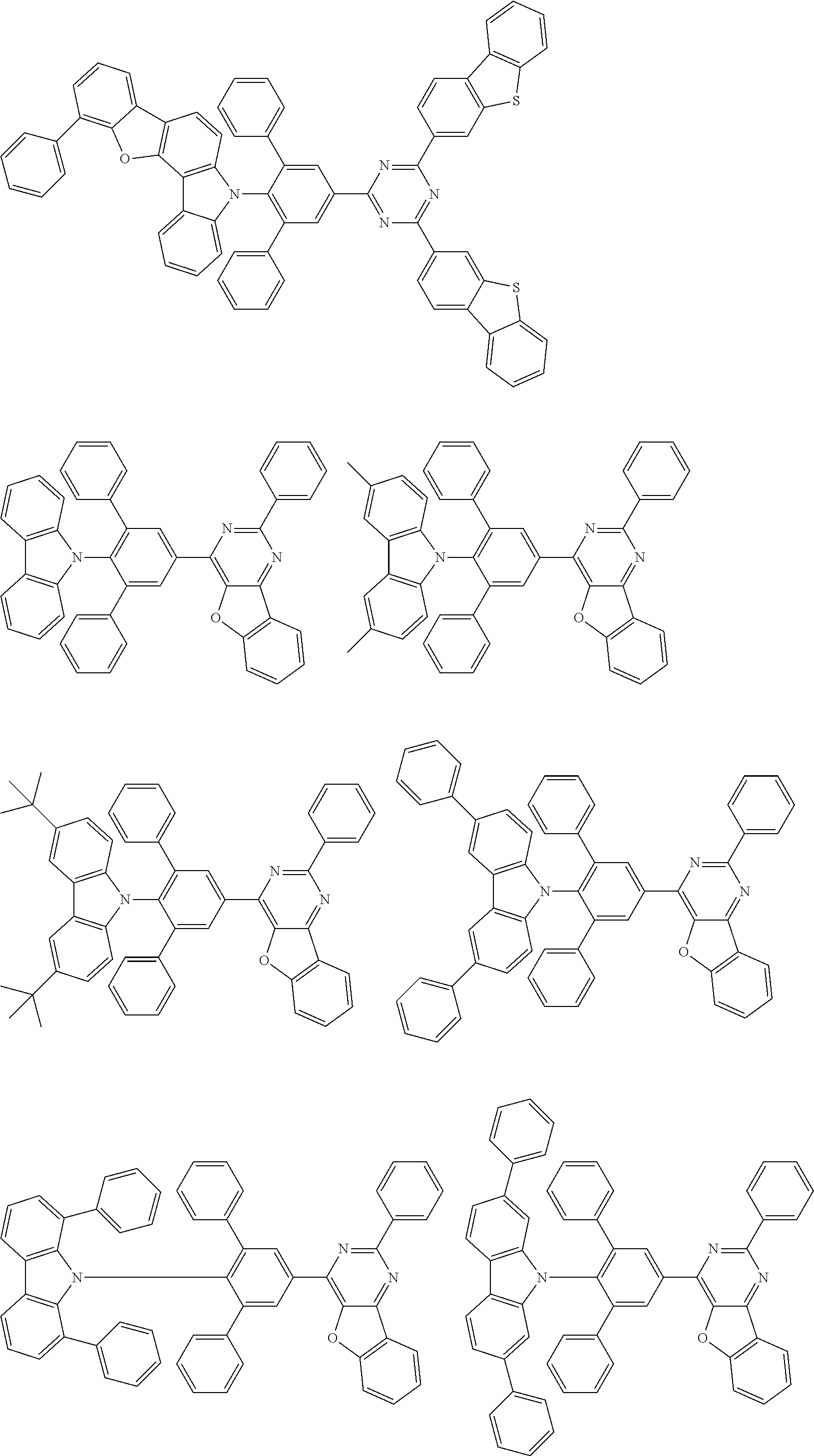

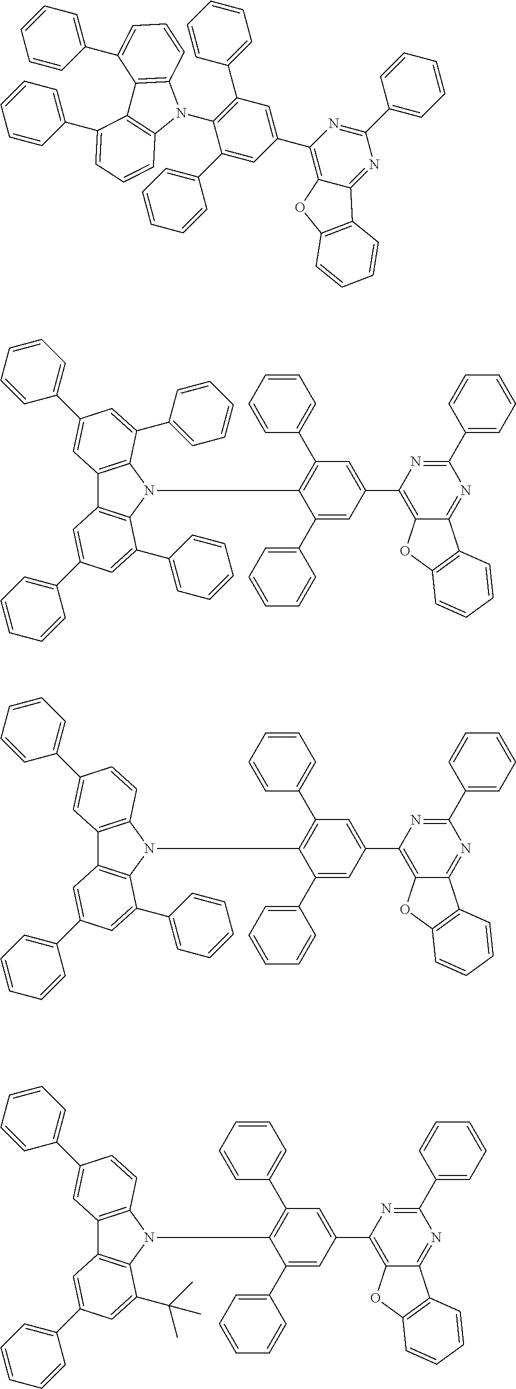 [SMILES: c1ccc(-c2nc(-c3cc(-c4ccccc4)c(-n4c5cccc(-c6ccccc6)c5c5c(-c6ccccc6)cccc54)c(-c4ccccc4)c3)c3oc4ccccc4c3n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc4c3oc3ccccc34)cc2-c2ccccc2)cc1 ; c1ccc(-c2ccc3c(c2)c2cc(-c4ccccc4)cc(-c4ccccc4)c2n3-c2c(-c3ccccc3)cc(-c3nc(-c4ccccc4)nc4c3oc3ccccc34)cc2-c2ccccc2)cc1 ; CC(C)(C)c1cc(-c2ccccc2)cc2c3cc(-c4ccccc4)ccc3n(-c3c(-c4ccccc4)cc(-c4nc(-c5ccccc5)nc5c4oc4ccccc45)cc3-c3ccccc3)c12]

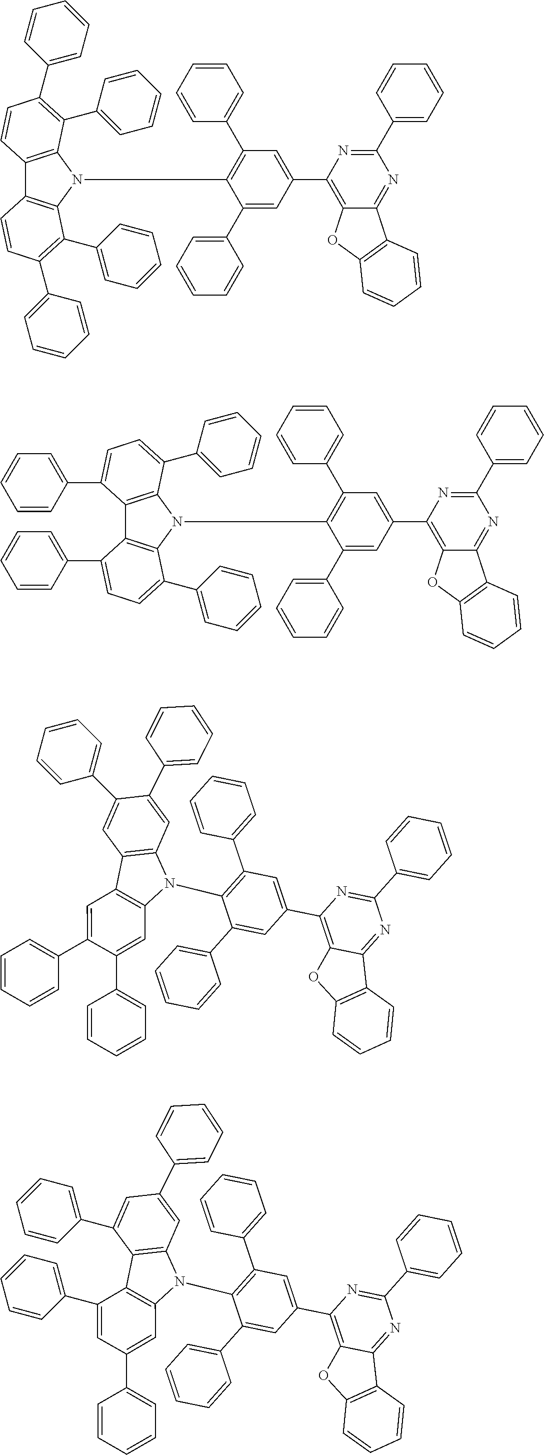

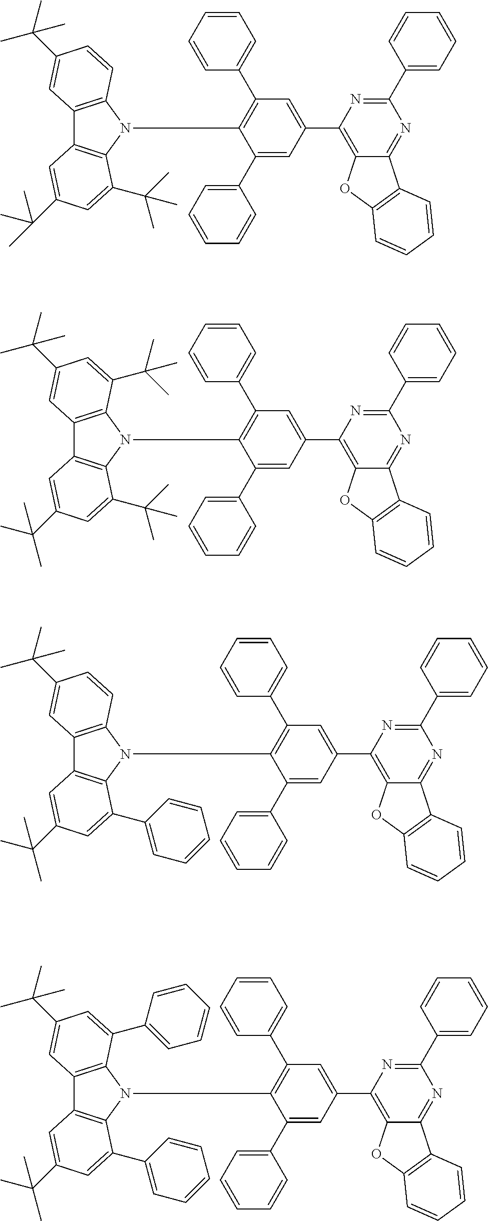 [SMILES: CC(C)(C)c1ccc2c(c1)c1cc(C(C)(C)C)cc(C(C)(C)C)c1n2-c1c(-c2ccccc2)cc(-c2nc(-c3ccccc3)nc3c2oc2ccccc23)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc3c2oc2ccccc23)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccccc3)nc3c2oc2ccccc23)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)nc3c2oc2ccccc23)cc1-c1ccccc1]

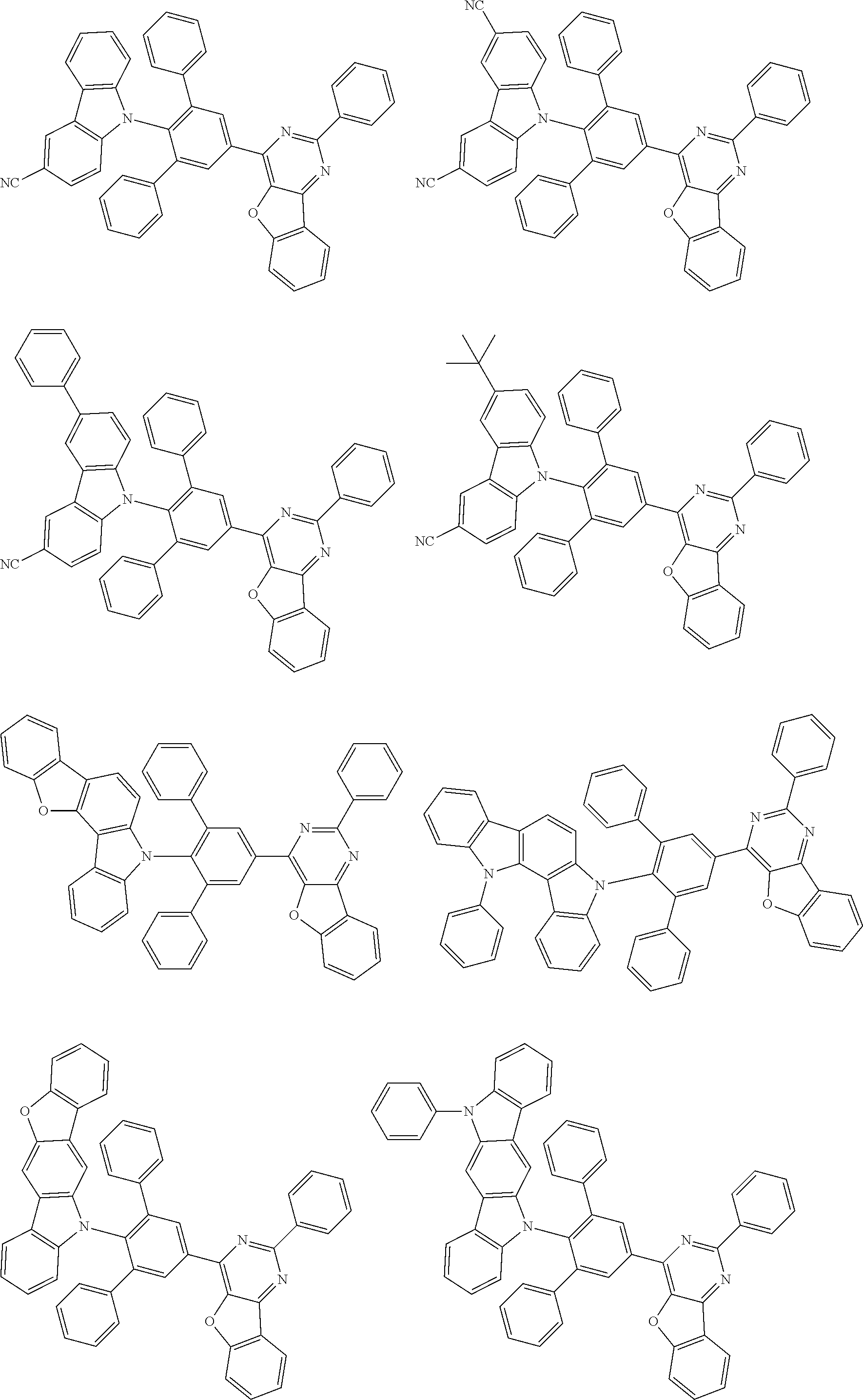

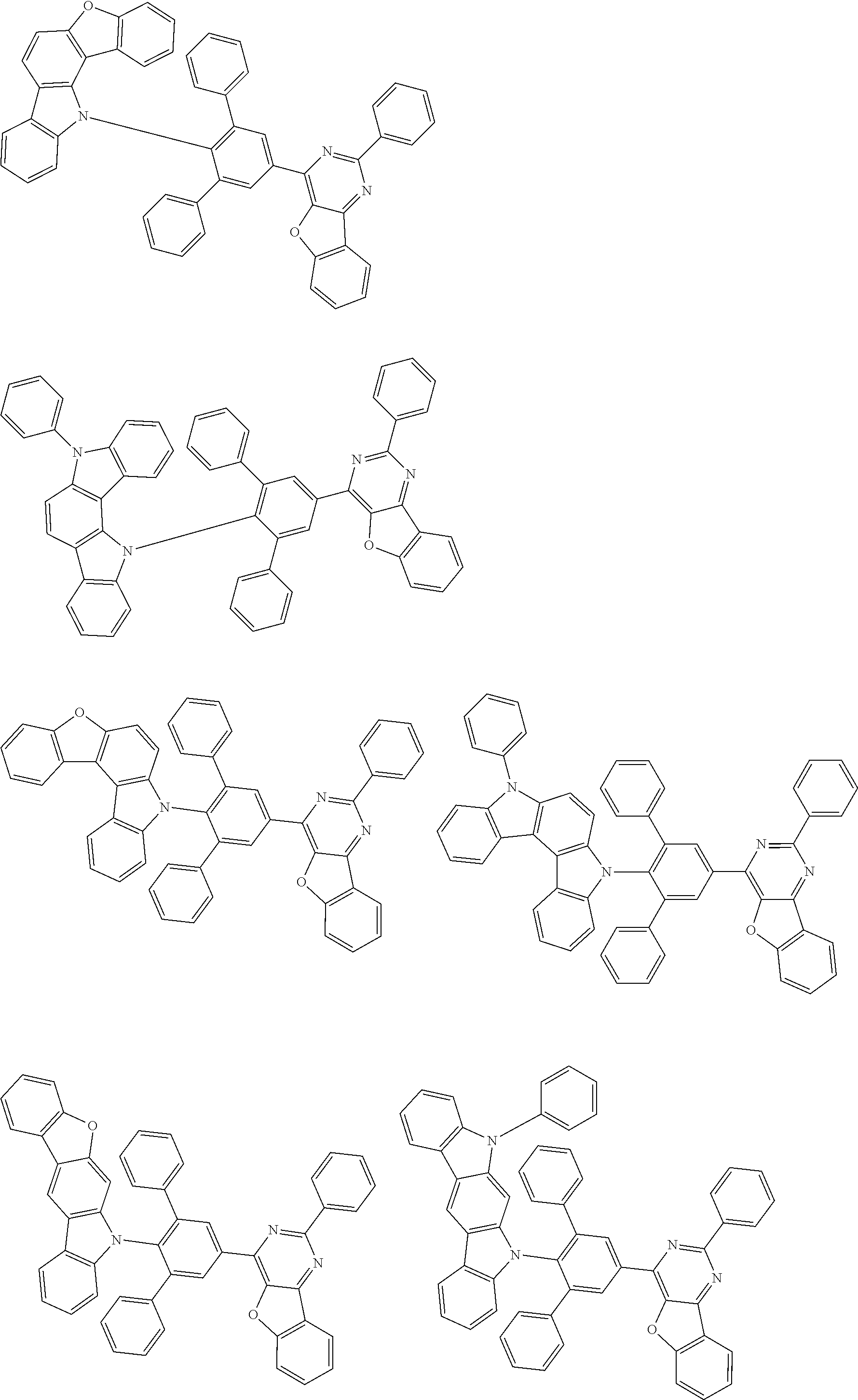

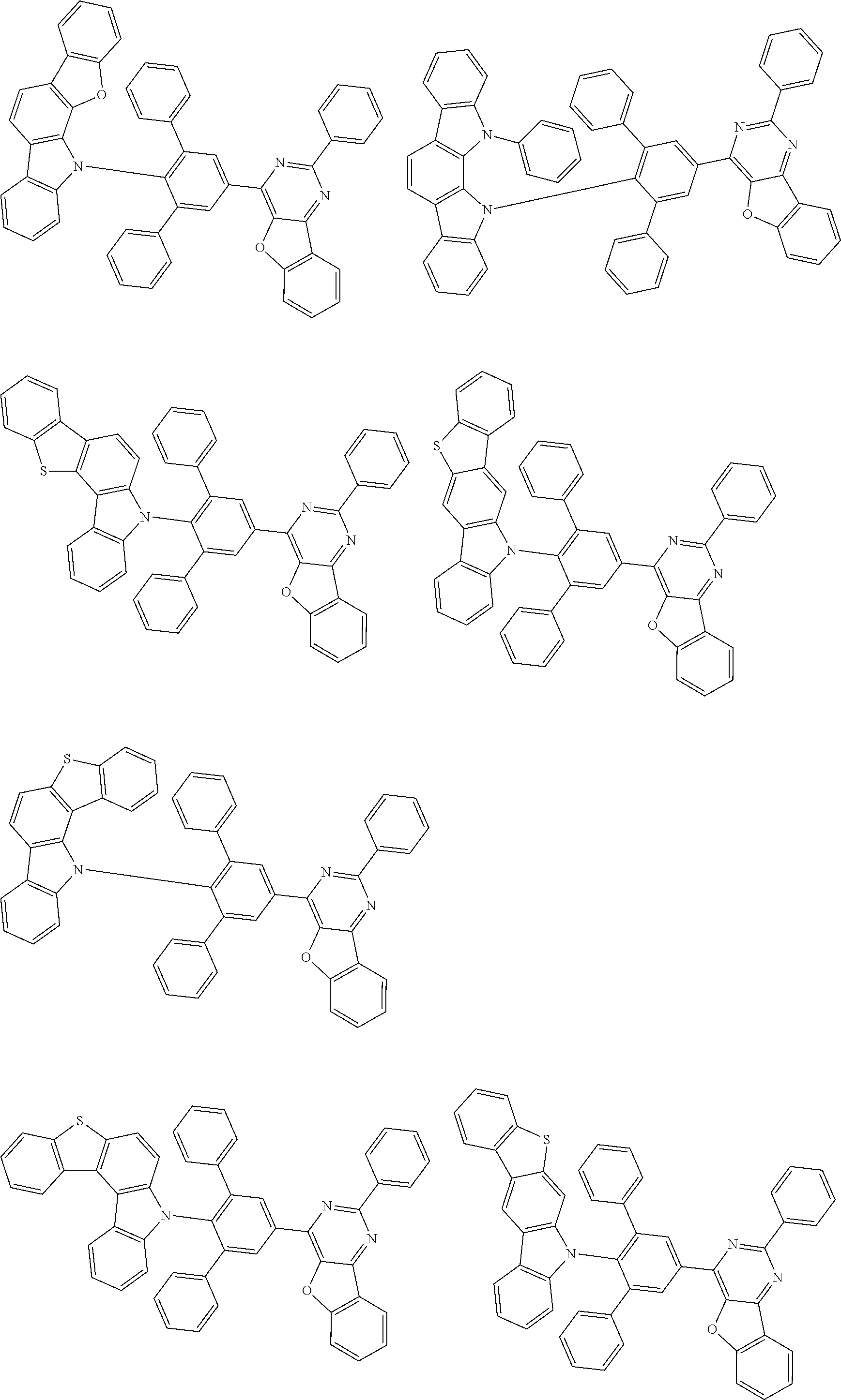

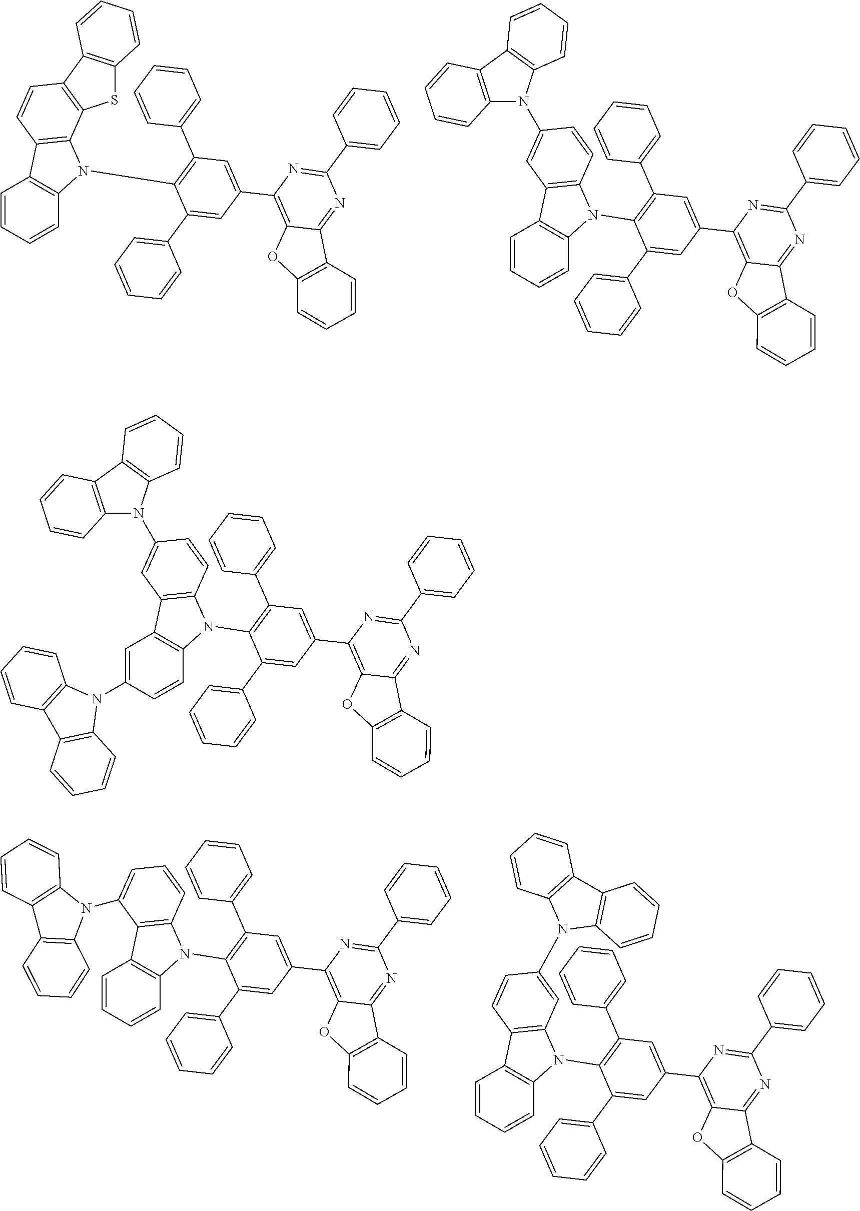

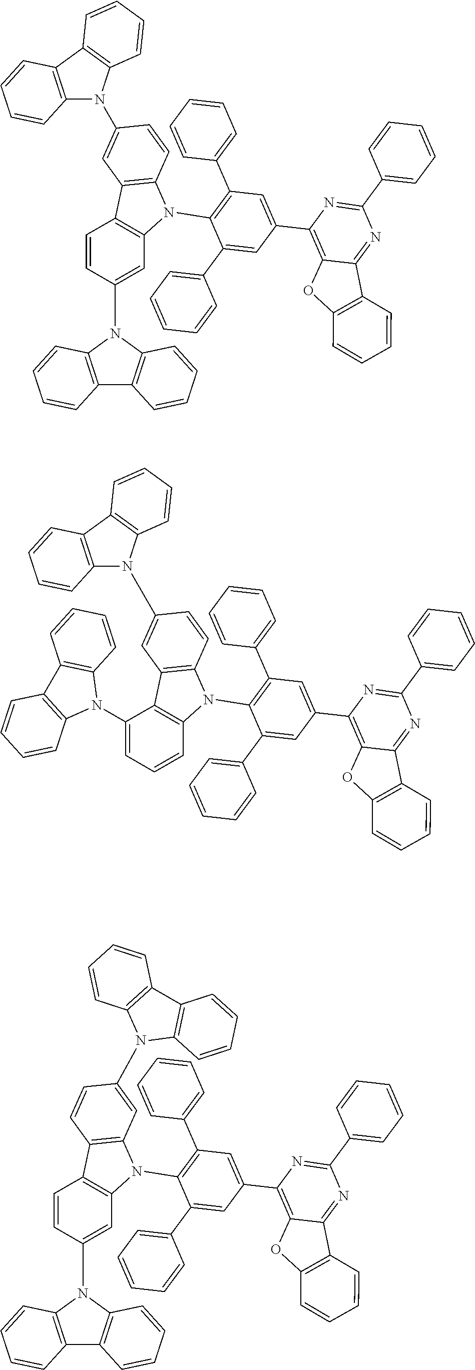 [SMILES: c1ccc(-c2nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)c3oc4ccccc4c3n2)cc1 ; c1ccc(-c2nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)c3oc4ccccc4c3n2)cc1 ; c1ccc(-c2nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)c3oc4ccccc4c3n2)cc1]

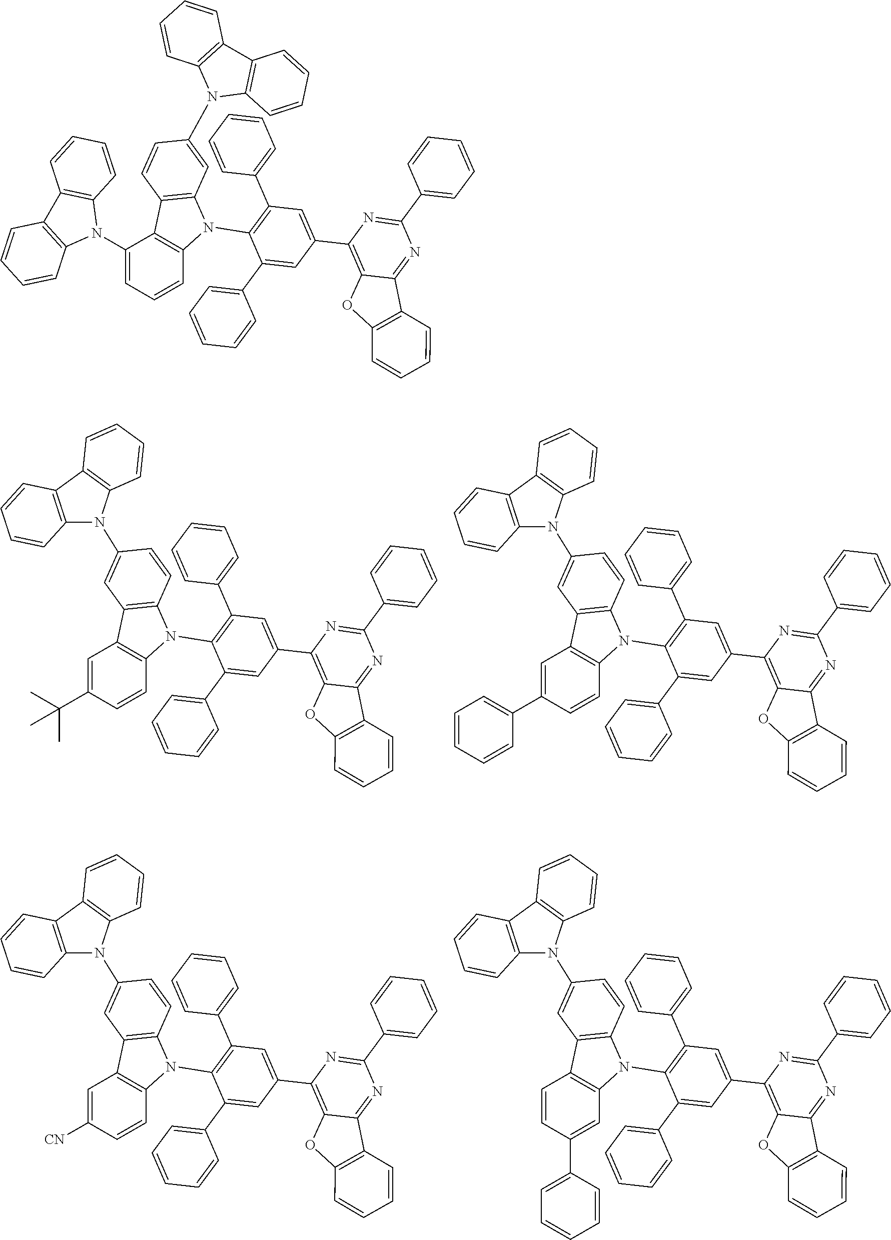

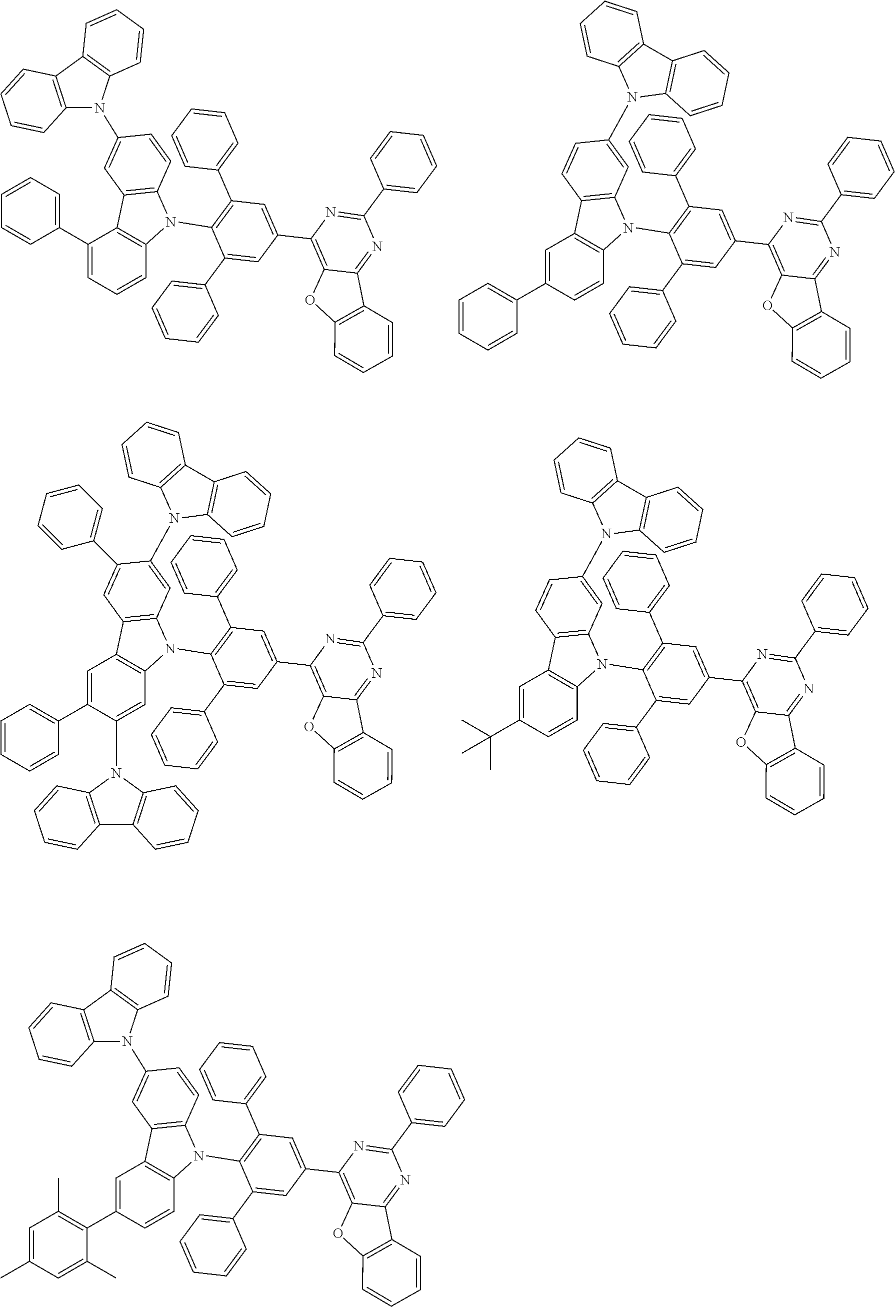

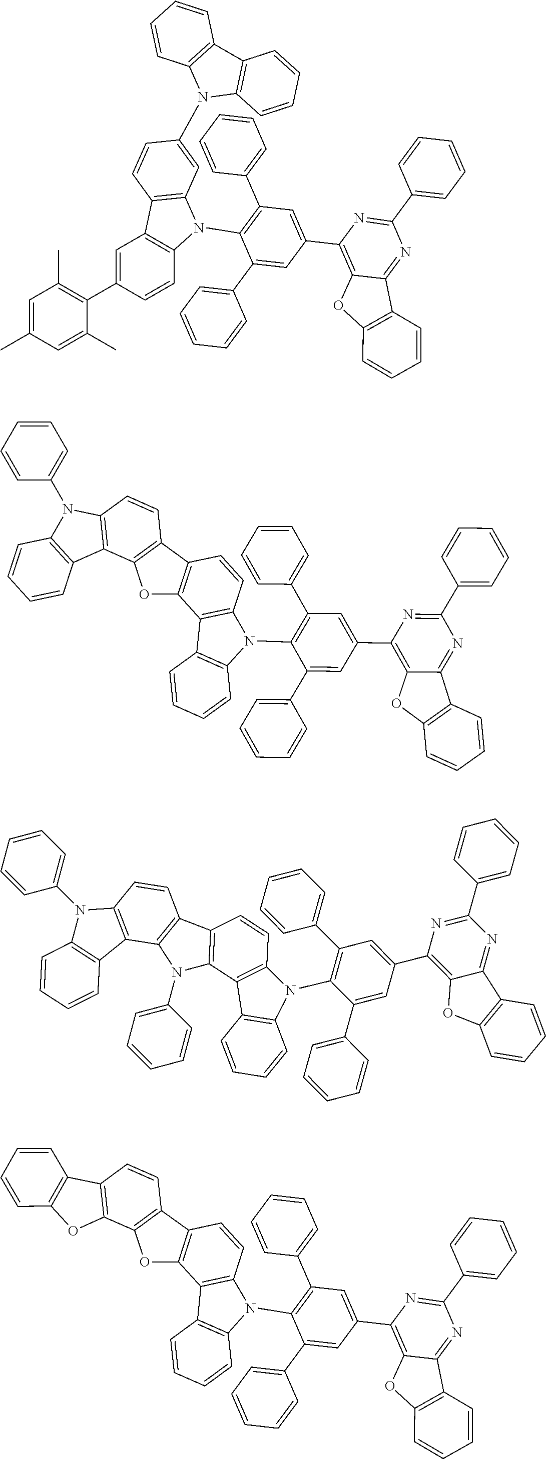 [SMILES: Cc1cc(C)c(-c2ccc3c(c2)c2ccc(-n4c5ccccc5c5ccccc54)cc2n3-c2c(-c3ccccc3)cc(-c3nc(-c4ccccc4)nc4c3oc3ccccc34)cc2-c2ccccc2)c(C)c1 ; c1ccc(-c2nc(-c3cc(-c4ccccc4)c(-n4c5ccccc5c5c6oc7c(ccc8c7c7ccccc7n8-c7ccccc7)c6ccc54)c(-c4ccccc4)c3)c3oc4ccccc4c3n2)cc1 ; c1ccc(-c2nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)c3oc4ccccc4c3n2)cc1 ; c1ccc(-c2nc(-c3cc(-c4ccccc4)c(-n4c5ccccc5c5c6oc7c(ccc8c9ccccc9oc87)c6ccc54)c(-c4ccccc4)c3)c3oc4ccccc4c3n2)cc1]

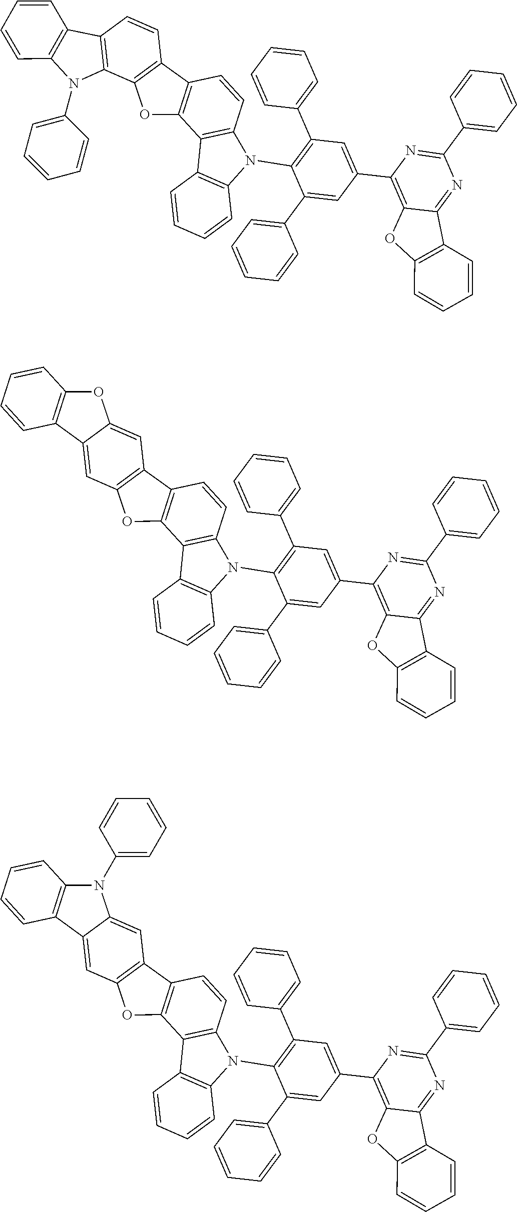 [SMILES: c1ccc(-c2nc(-c3cc(-c4ccccc4)c(-n4c5ccccc5c5c6oc7c(ccc8c9ccccc9n(-c9ccccc9)c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7cc8c(cc7c6ccc54)oc4ccccc48)c(-c4ccccc4)c3)c3oc4ccccc4c3n2)cc1 ; c1ccc(-c2nc(-c3cc(-c4ccccc4)c(-n4c5ccccc5c5c6oc7cc8c9ccccc9n(-c9ccccc9)c8cc7c6ccc54)c(-c4ccccc4)c3)c3oc4ccccc4c3n2)cc1]

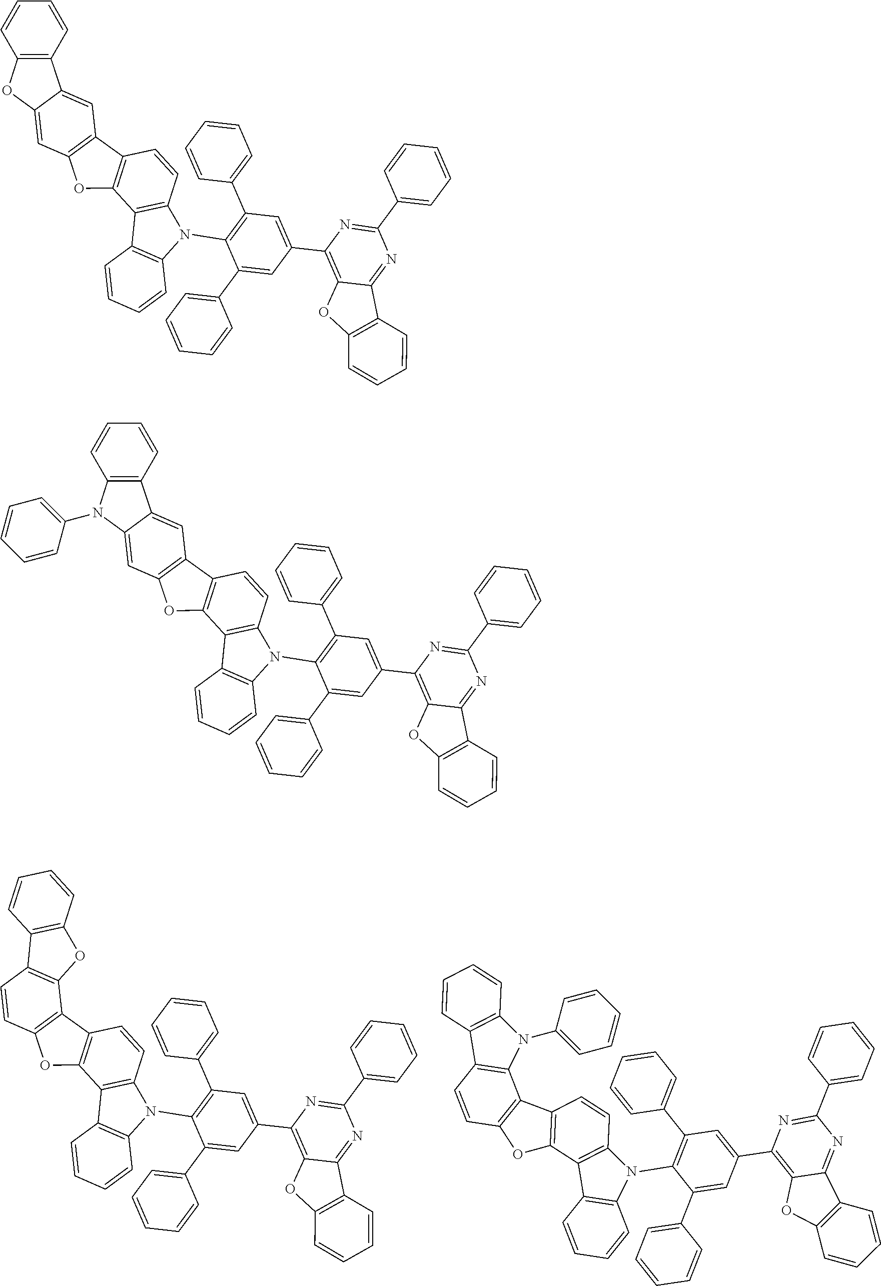

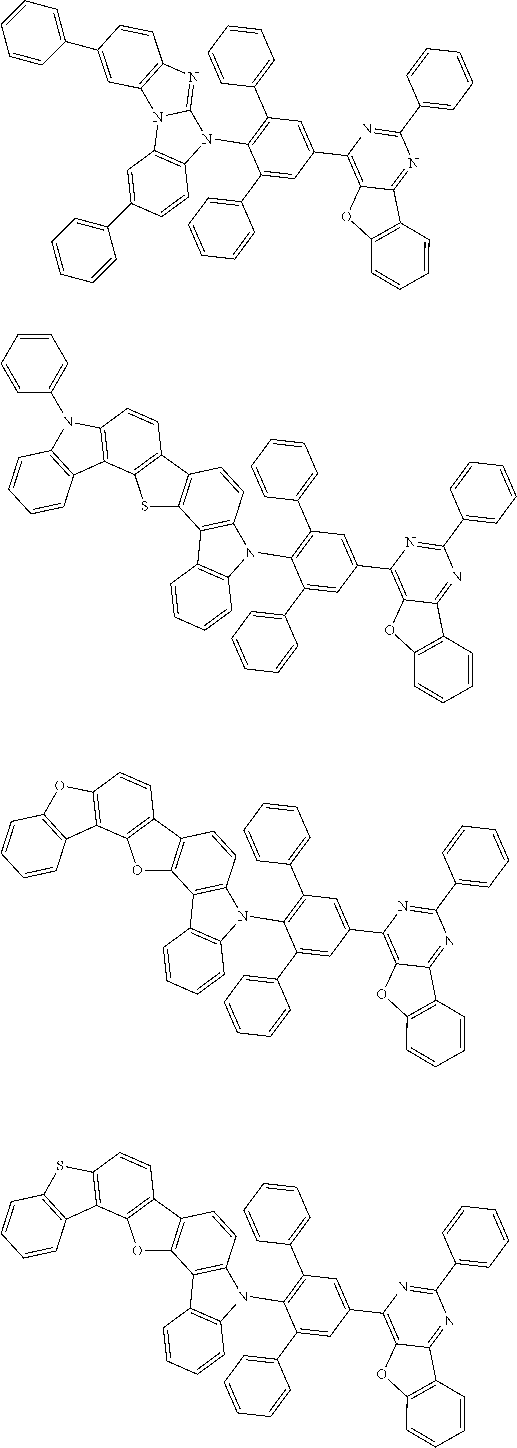 [SMILES: c1ccc(-c2ccc3nc4n(-c5c(-c6ccccc6)cc(-c6nc(-c7ccccc7)nc7c6oc6ccccc67)cc5-c5ccccc5)c5ccc(-c6ccccc6)cc5n4c3c2)cc1 ; c1ccc(-c2nc(-c3cc(-c4ccccc4)c(-n4c5ccccc5c5c6sc7c(ccc8c7c7ccccc7n8-c7ccccc7)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8oc9ccccc9c87)c6ccc54)c(-c4ccccc4)c3)c3oc4ccccc4c3n2)cc1 ; c1ccc(-c2nc(-c3cc(-c4ccccc4)c(-n4c5ccccc5c5c6oc7c(ccc8sc9ccccc9c87)c6ccc54)c(-c4ccccc4)c3)c3oc4ccccc4c3n2)cc1]

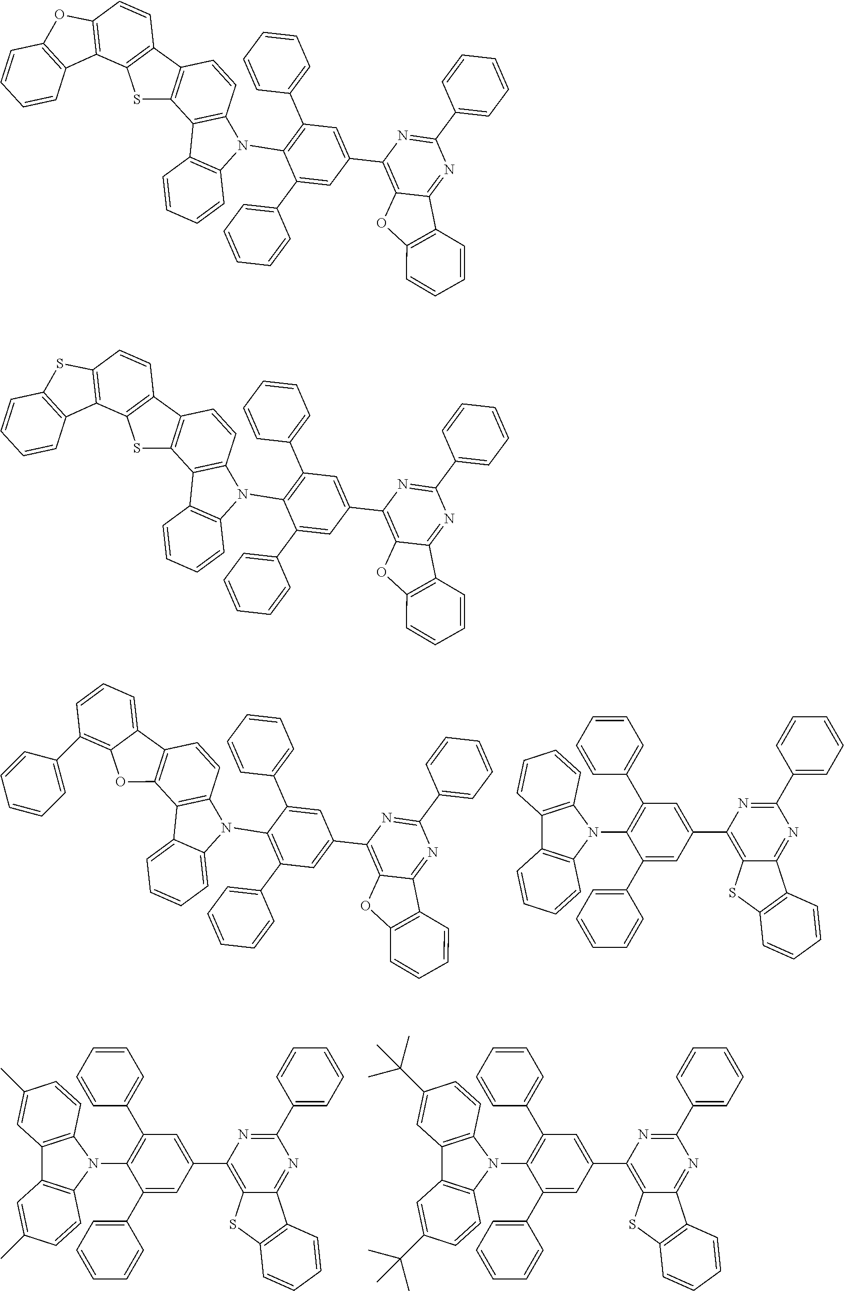

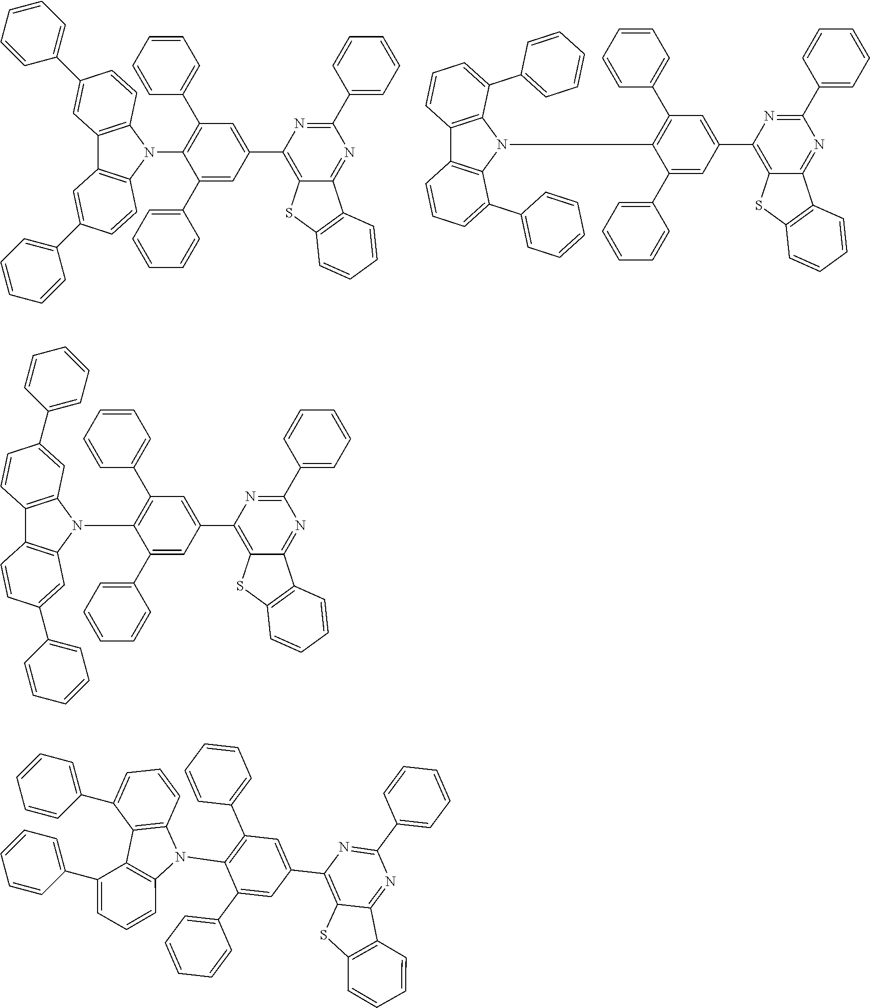

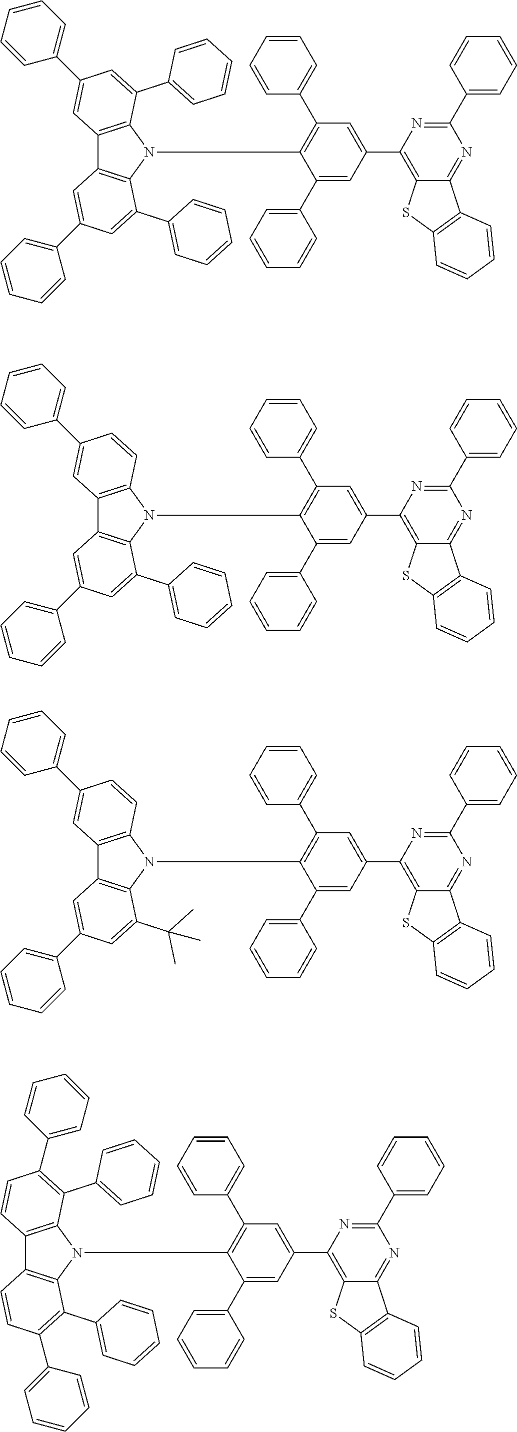

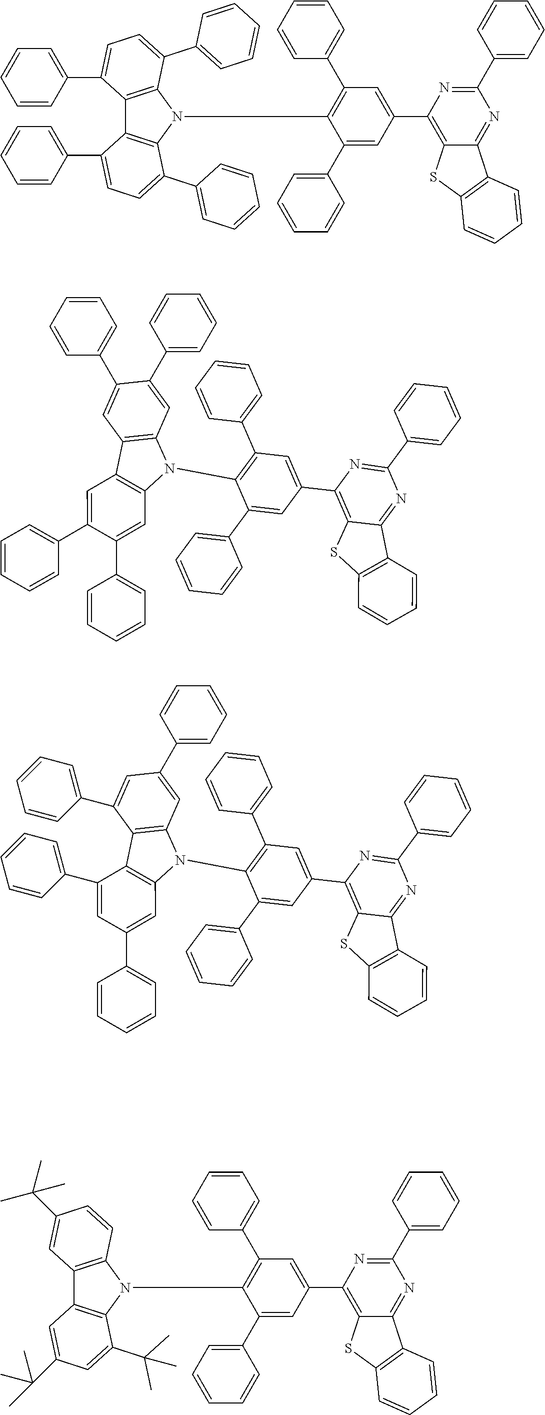

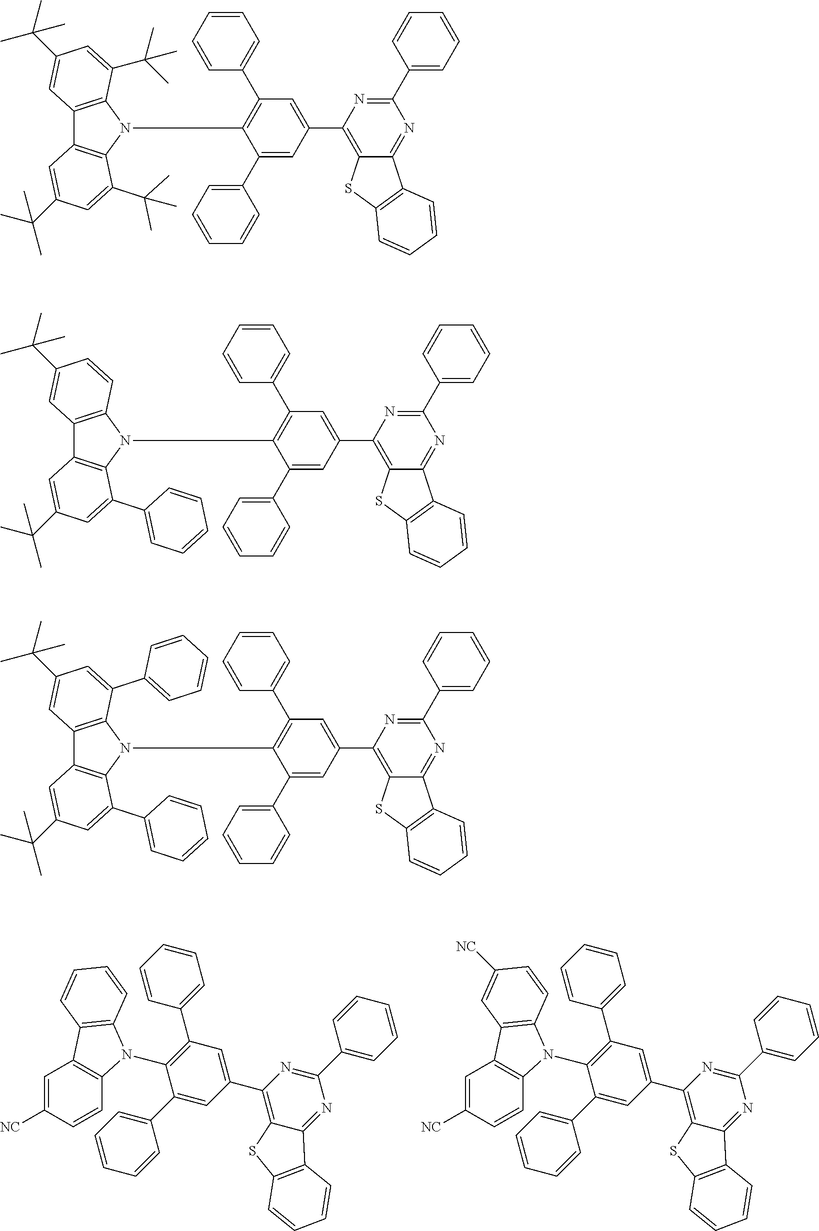

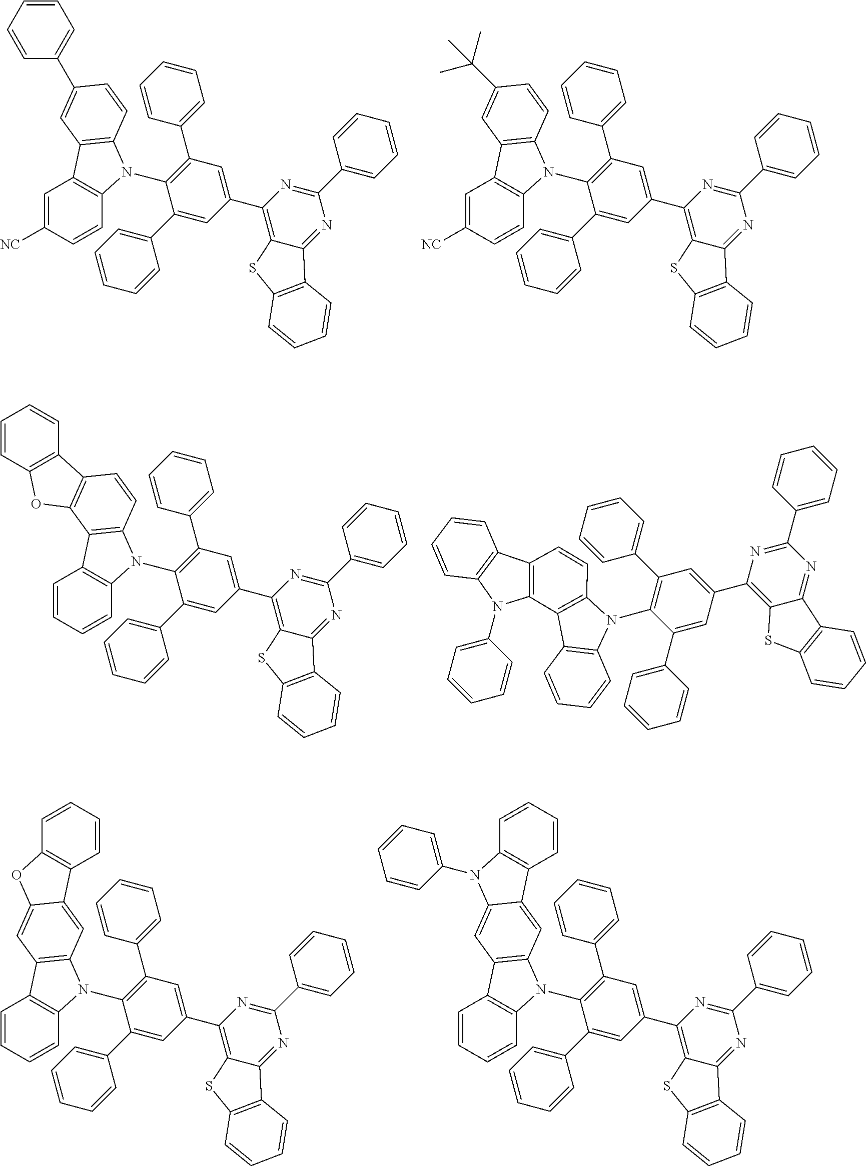

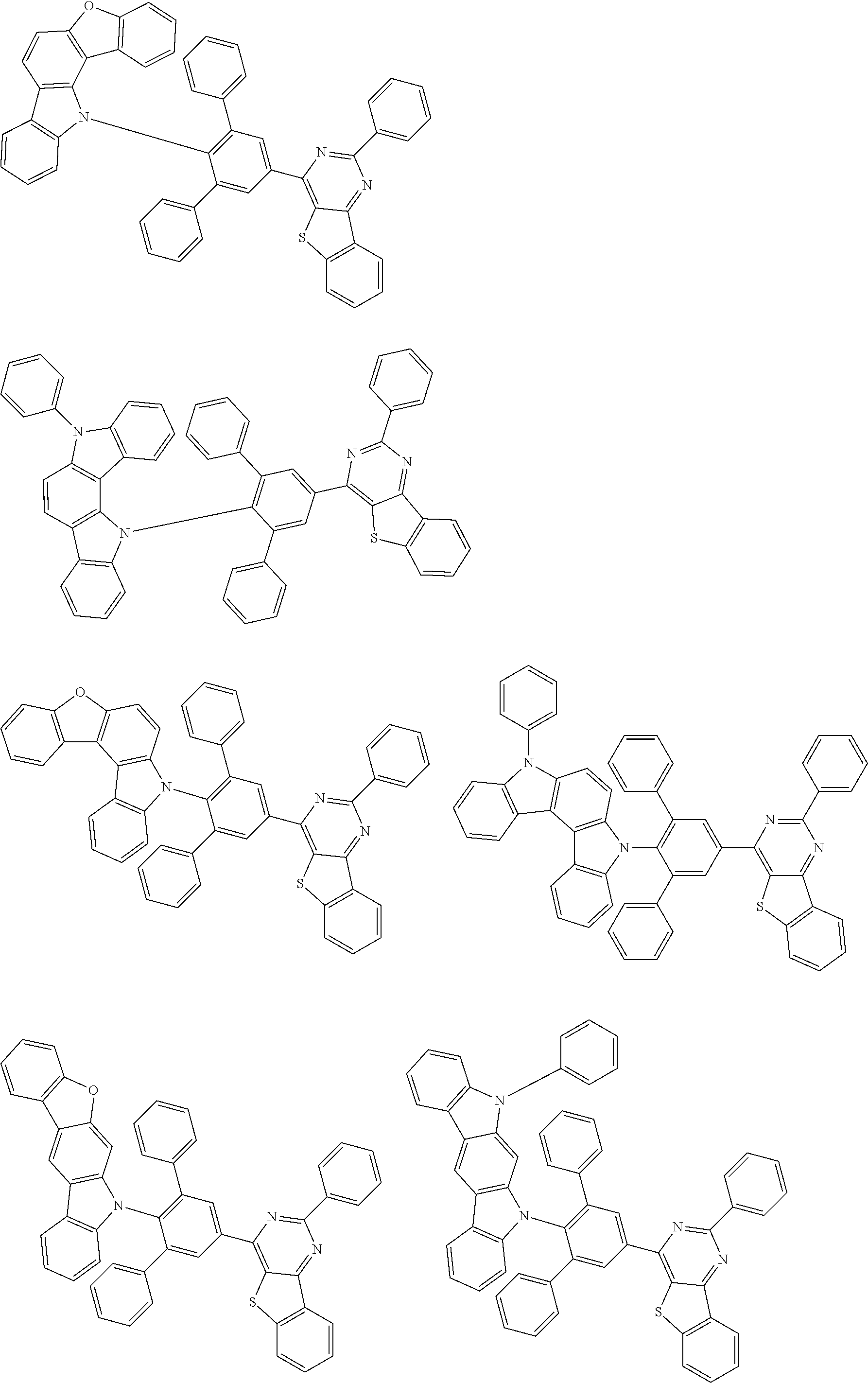

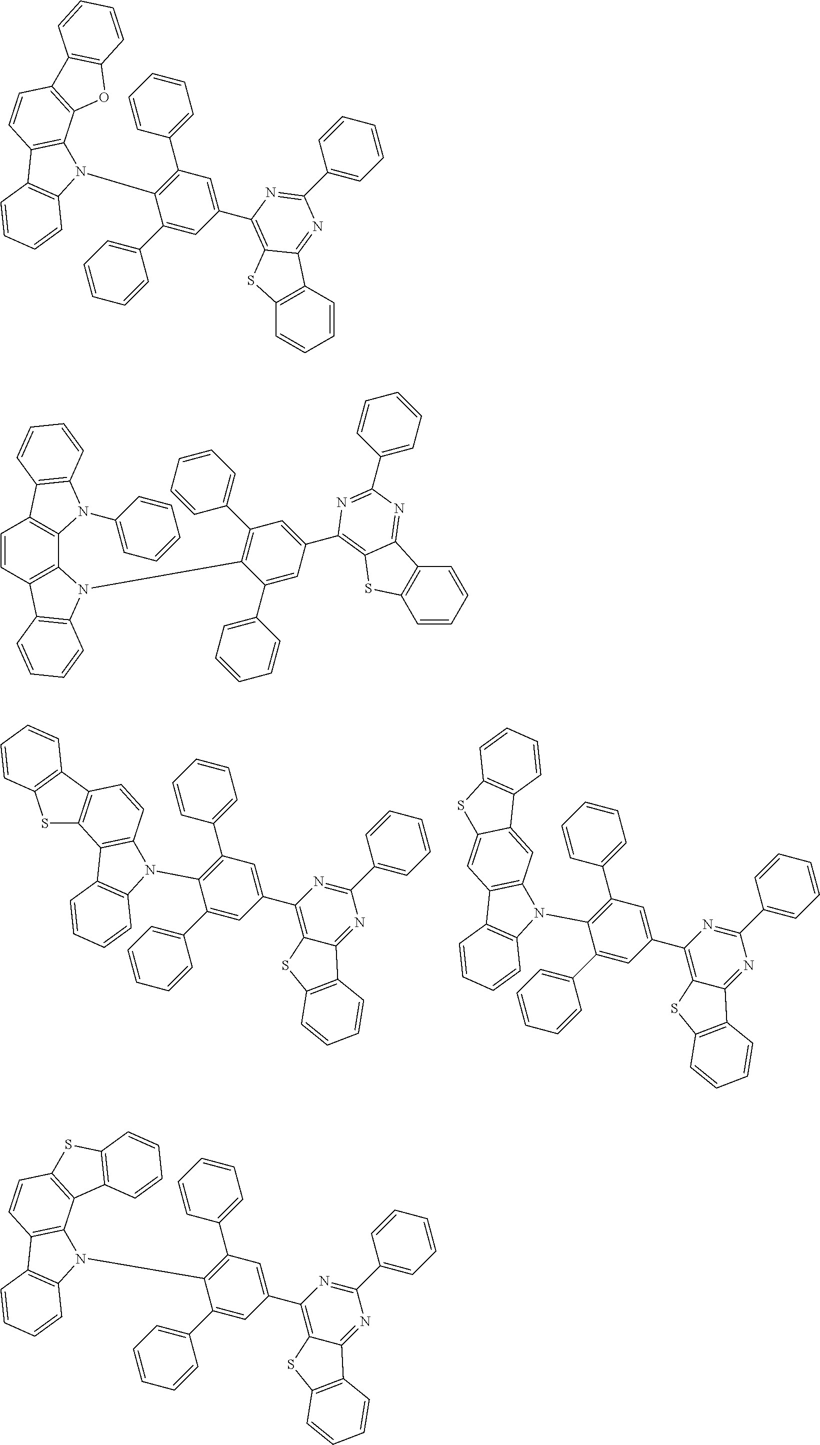

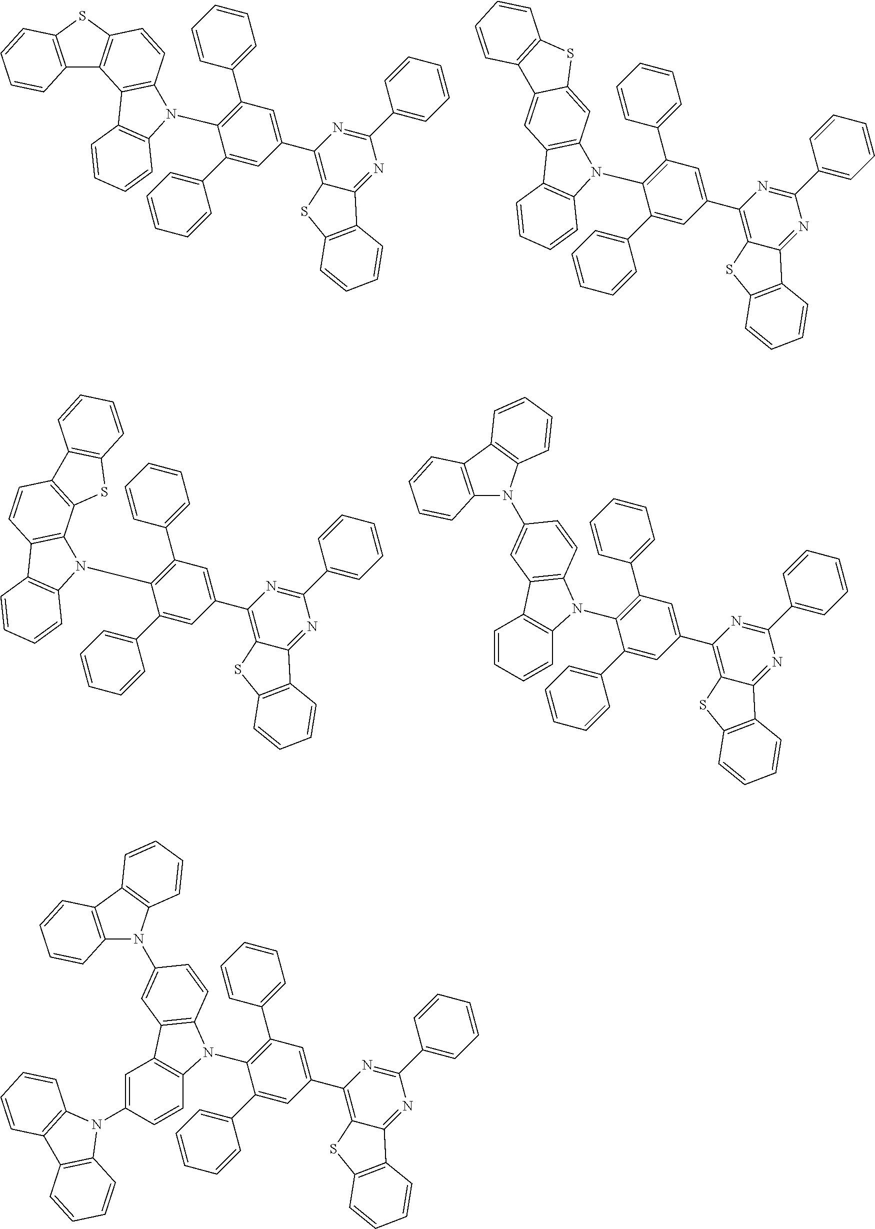

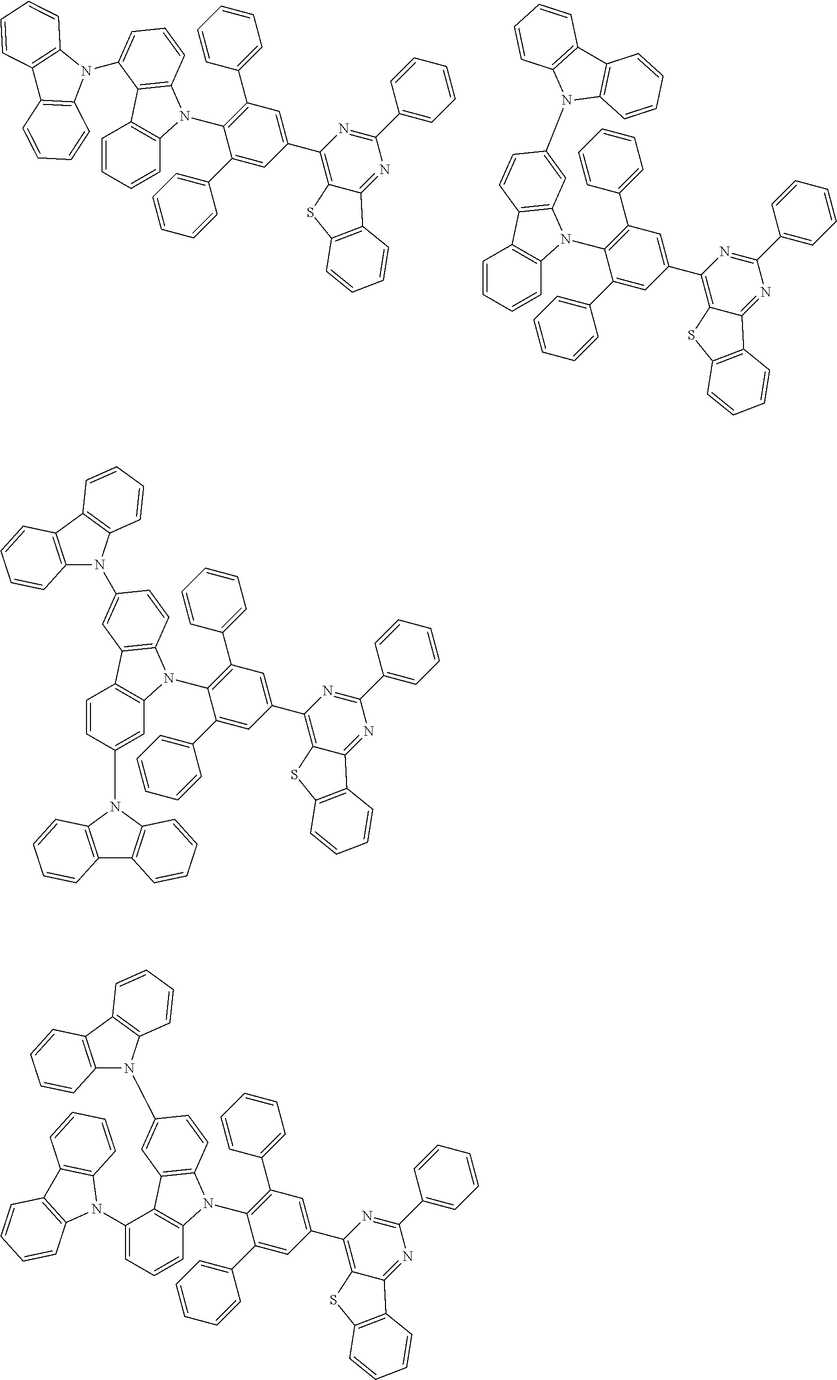 [SMILES: c1ccc(-c2nc(-c3cc(-c4ccccc4)c(-n4c5ccccc5c5c(-n6c7ccccc7c7ccccc76)cccc54)c(-c4ccccc4)c3)c3sc4ccccc4c3n2)cc1 ; c1ccc(-c2nc(-c3cc(-c4ccccc4)c(-n4c5ccccc5c5ccc(-n6c7ccccc7c7ccccc76)cc54)c(-c4ccccc4)c3)c3sc4ccccc4c3n2)cc1 ; c1ccc(-c2nc(-c3cc(-c4ccccc4)c(-n4c5ccc(-n6c7ccccc7c7ccccc76)cc5c5ccc(-n6c7ccccc7c7ccccc76)cc54)c(-c4ccccc4)c3)c3sc4ccccc4c3n2)cc1 ; c1ccc(-c2nc(-c3cc(-c4ccccc4)c(-n4c5ccc(-n6c7ccccc7c7ccccc76)cc5c5c(-n6c7ccccc7c7ccccc76)cccc54)c(-c4ccccc4)c3)c3sc4ccccc4c3n2)cc1]

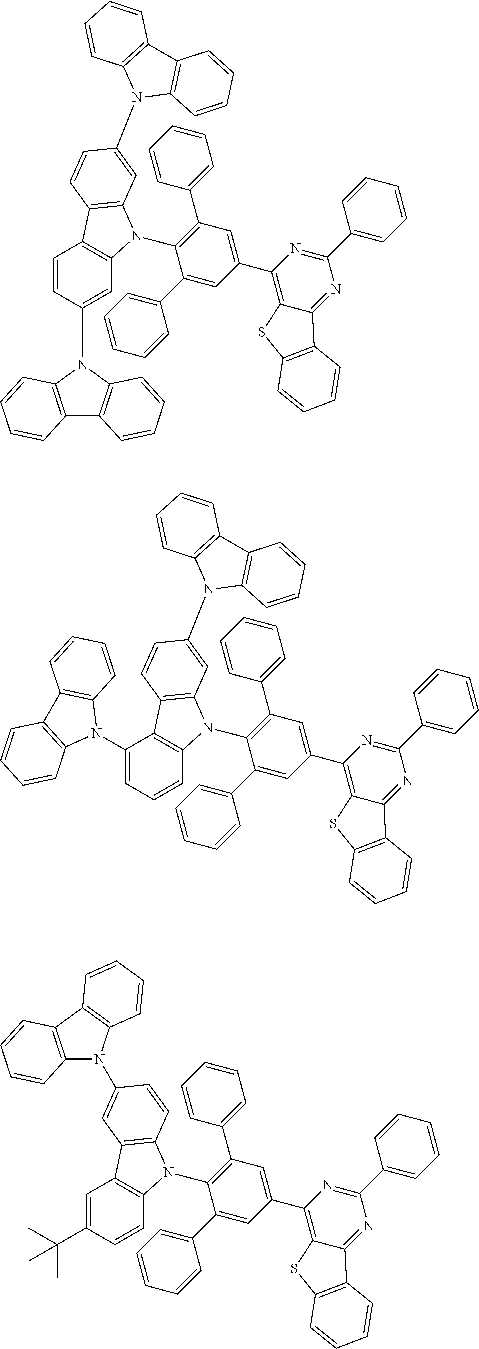 [SMILES: c1ccc(-c2nc(-c3cc(-c4ccccc4)c(-n4c5cc(-n6c7ccccc7c7ccccc76)ccc5c5ccc(-n6c7ccccc7c7ccccc76)cc54)c(-c4ccccc4)c3)c3sc4ccccc4c3n2)cc1 ; c1ccc(-c2nc(-c3cc(-c4ccccc4)c(-n4c5cc(-n6c7ccccc7c7ccccc76)ccc5c5c(-n6c7ccccc7c7ccccc76)cccc54)c(-c4ccccc4)c3)c3sc4ccccc4c3n2)cc1 ; CC(C)(C)c1ccc2c(c1)c1cc(-n3c4ccccc4c4ccccc43)ccc1n2-c1c(-c2ccccc2)cc(-c2nc(-c3ccccc3)nc3c2sc2ccccc23)cc1-c1ccccc1]

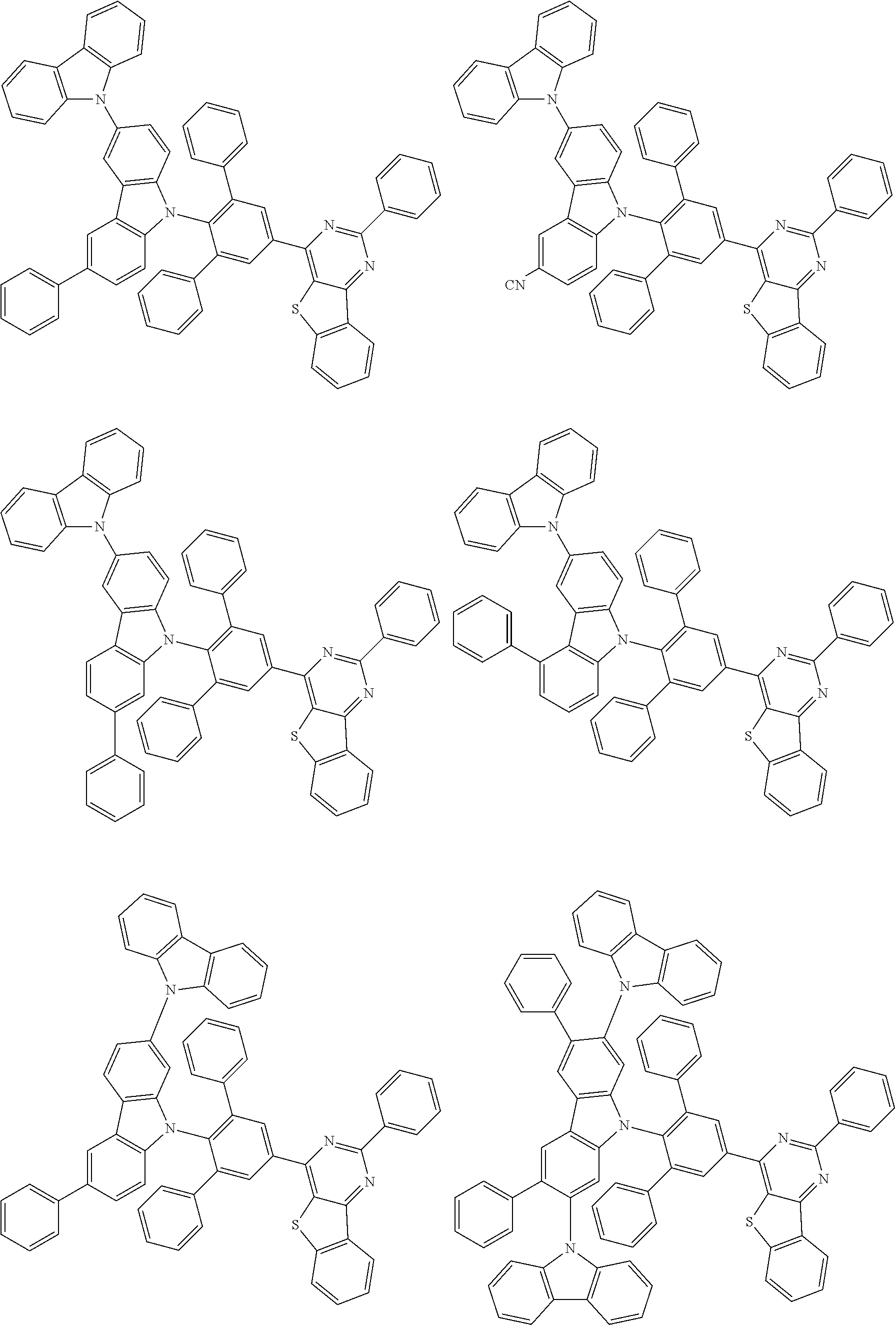

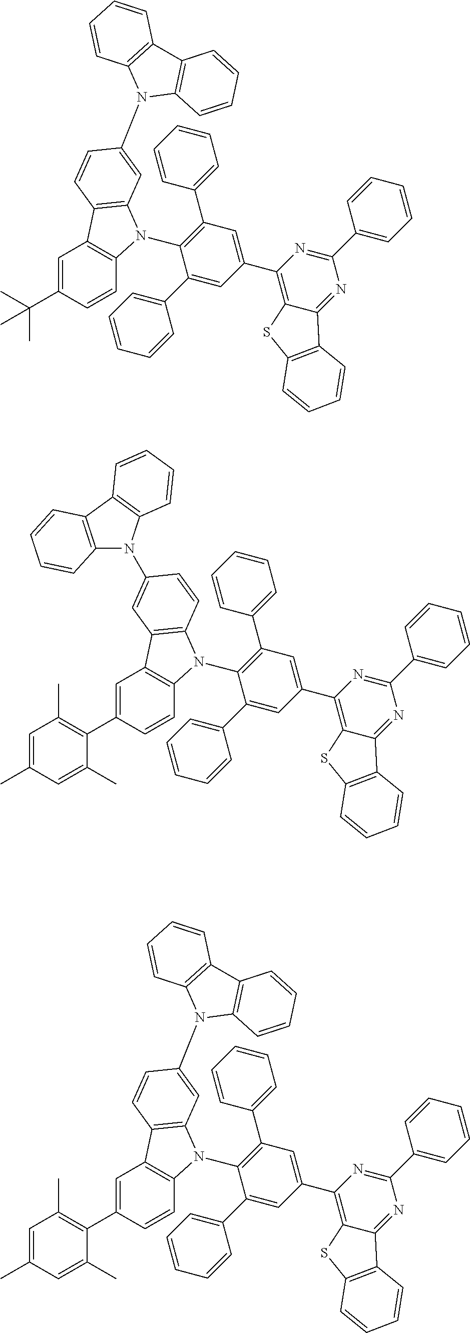 [SMILES: CC(C)(C)c1ccc2c(c1)c1ccc(-n3c4ccccc4c4ccccc43)cc1n2-c1c(-c2ccccc2)cc(-c2nc(-c3ccccc3)nc3c2sc2ccccc23)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccccc4)nc4c3sc3ccccc34)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccccc4)nc4c3sc3ccccc34)cc2-c2ccccc2)c(C)c1]

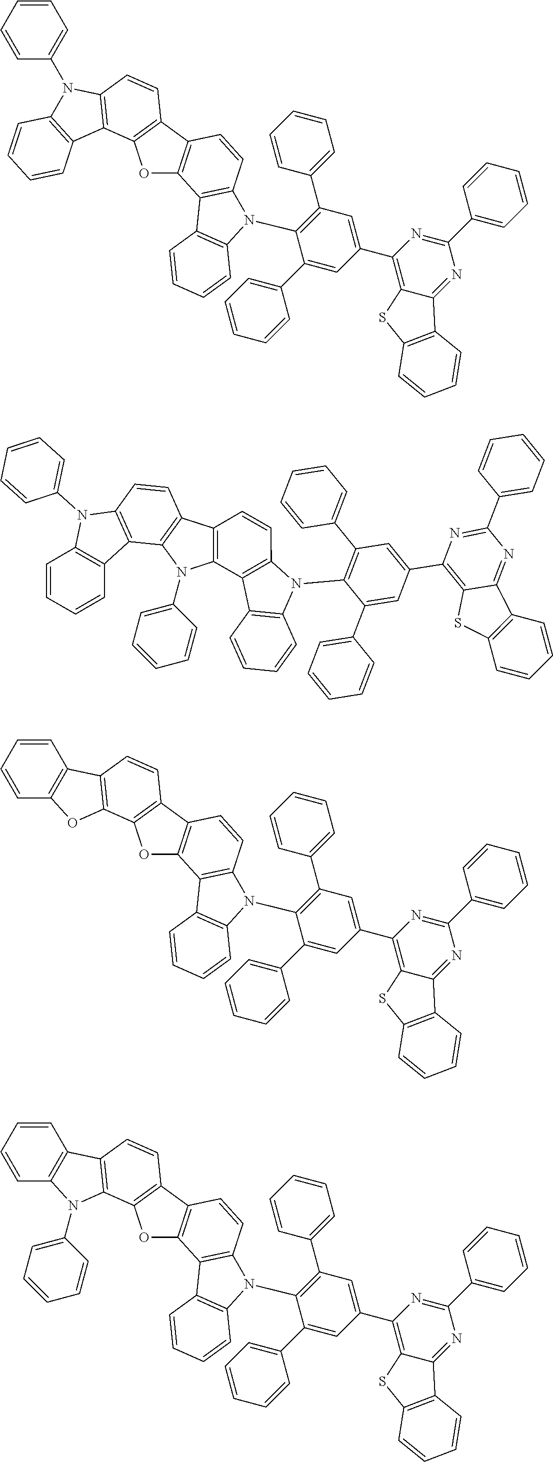 [SMILES: c1ccc(-c2nc(-c3cc(-c4ccccc4)c(-n4c5ccccc5c5c6oc7c(ccc8c7c7ccccc7n8-c7ccccc7)c6ccc54)c(-c4ccccc4)c3)c3sc4ccccc4c3n2)cc1 ; c1ccc(-c2nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)c3sc4ccccc4c3n2)cc1 ; c1ccc(-c2nc(-c3cc(-c4ccccc4)c(-n4c5ccccc5c5c6oc7c(ccc8c9ccccc9oc87)c6ccc54)c(-c4ccccc4)c3)c3sc4ccccc4c3n2)cc1 ; c1ccc(-c2nc(-c3cc(-c4ccccc4)c(-n4c5ccccc5c5c6oc7c(ccc8c9ccccc9n(-c9ccccc9)c87)c6ccc54)c(-c4ccccc4)c3)c3sc4ccccc4c3n2)cc1]

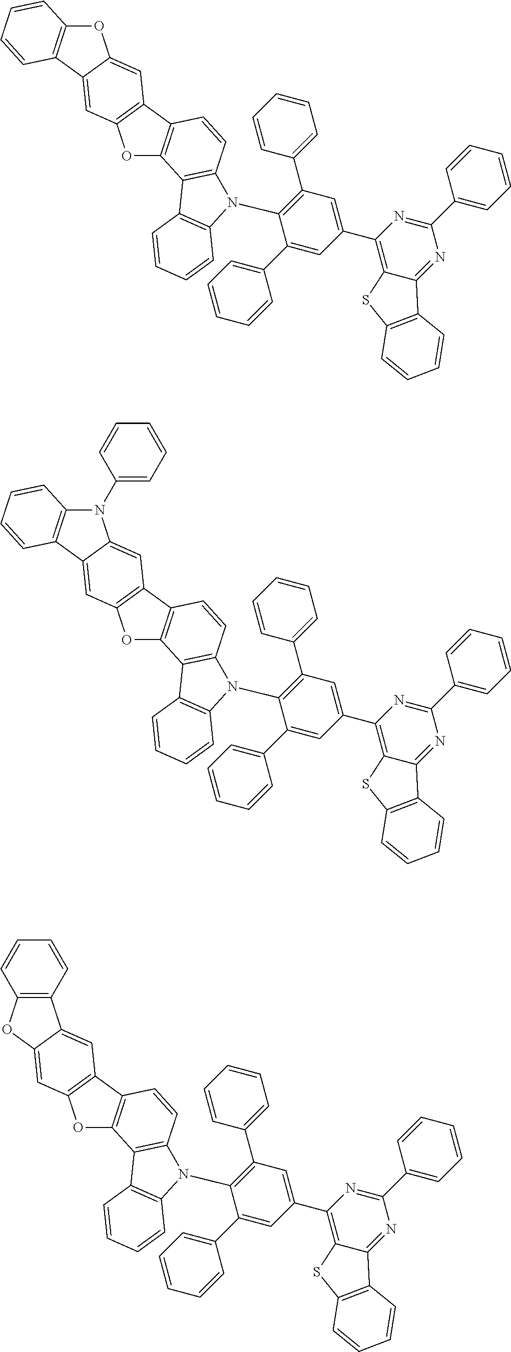 [SMILES: c1ccc(-c2nc(-c3cc(-c4ccccc4)c(-n4c5ccccc5c5c6oc7cc8c(cc7c6ccc54)oc4ccccc48)c(-c4ccccc4)c3)c3sc4ccccc4c3n2)cc1 ; c1ccc(-c2nc(-c3cc(-c4ccccc4)c(-n4c5ccccc5c5c6oc7cc8c9ccccc9n(-c9ccccc9)c8cc7c6ccc54)c(-c4ccccc4)c3)c3sc4ccccc4c3n2)cc1 ; c1ccc(-c2nc(-c3cc(-c4ccccc4)c(-n4c5ccccc5c5c6oc7cc8oc9ccccc9c8cc7c6ccc54)c(-c4ccccc4)c3)c3sc4ccccc4c3n2)cc1]

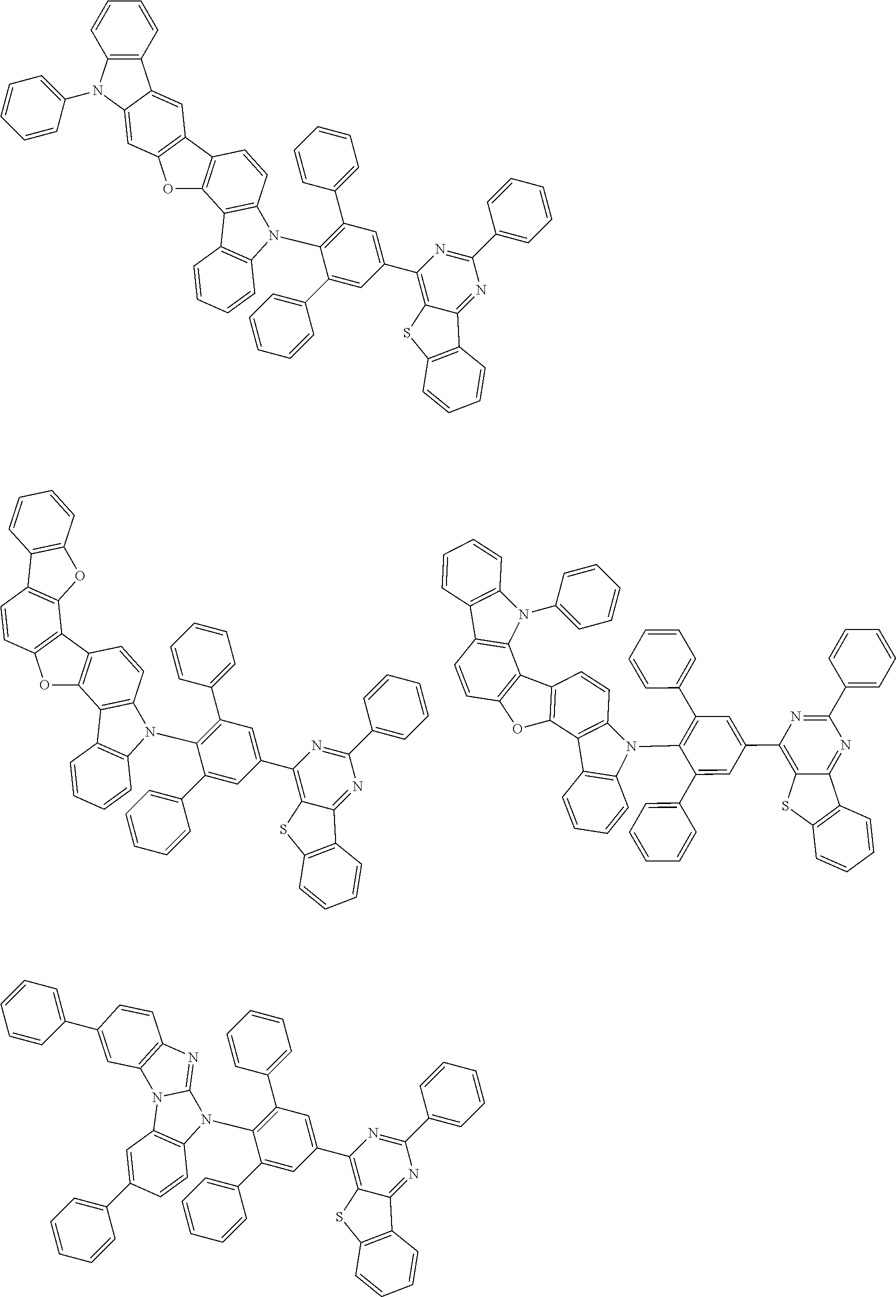

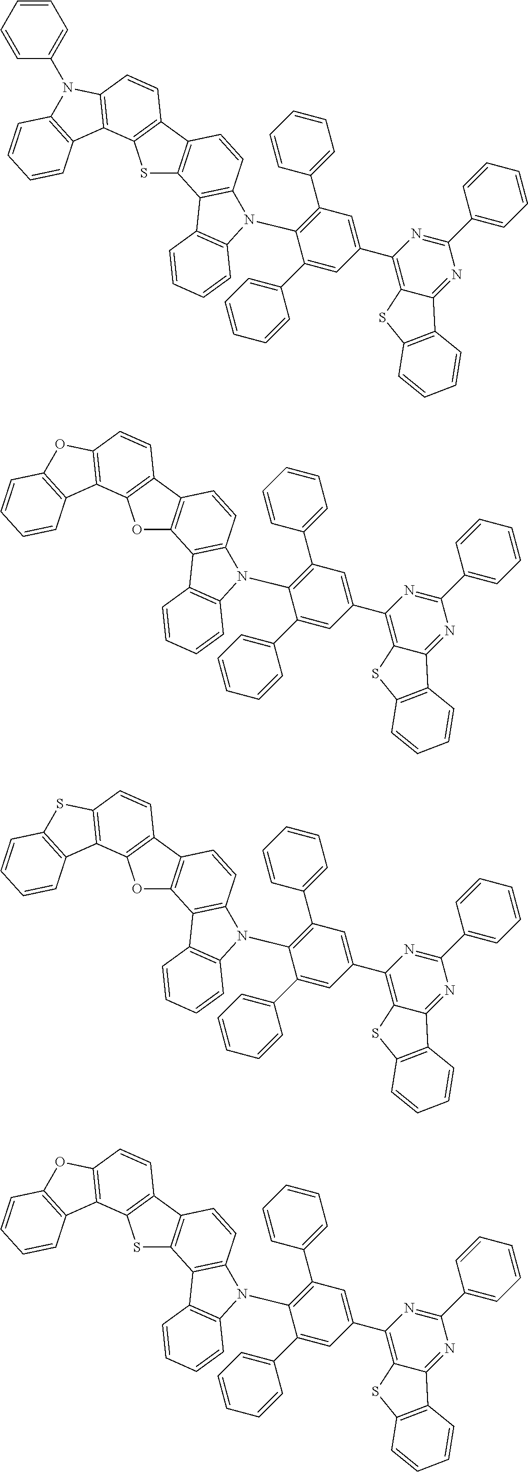 [SMILES: c1ccc(-c2nc(-c3cc(-c4ccccc4)c(-n4c5ccccc5c5c6sc7c(ccc8c7c7ccccc7n8-c7ccccc7)c6ccc54)c(-c4ccccc4)c3)c3sc4ccccc4c3n2)cc1 ; c1ccc(-c2nc(-c3cc(-c4ccccc4)c(-n4c5ccccc5c5c6oc7c(ccc8oc9ccccc9c87)c6ccc54)c(-c4ccccc4)c3)c3sc4ccccc4c3n2)cc1 ; c1ccc(-c2nc(-c3cc(-c4ccccc4)c(-n4c5ccccc5c5c6oc7c(ccc8sc9ccccc9c87)c6ccc54)c(-c4ccccc4)c3)c3sc4ccccc4c3n2)cc1 ; c1ccc(-c2nc(-c3cc(-c4ccccc4)c(-n4c5ccccc5c5c6sc7c(ccc8oc9ccccc9c87)c6ccc54)c(-c4ccccc4)c3)c3sc4ccccc4c3n2)cc1]

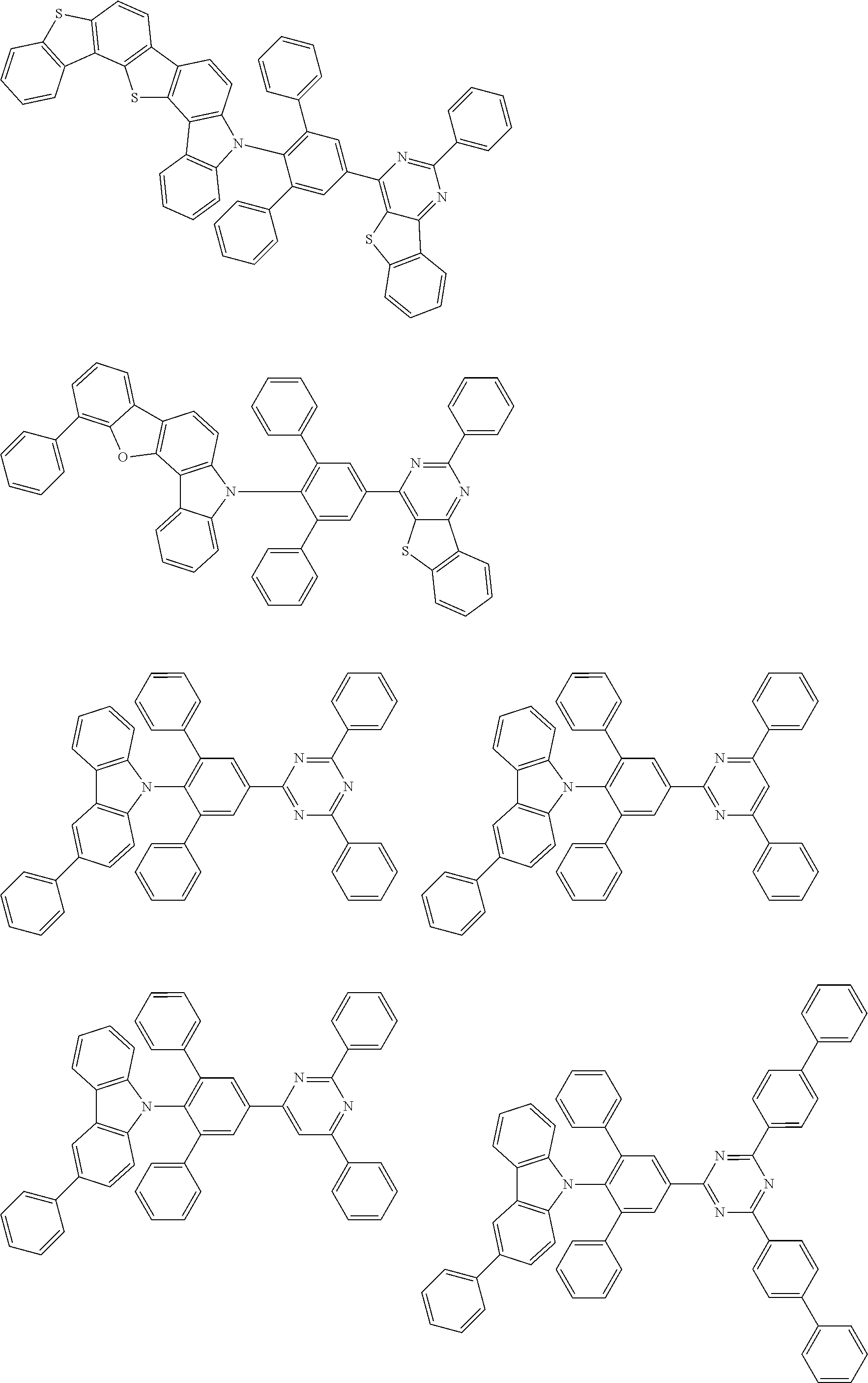

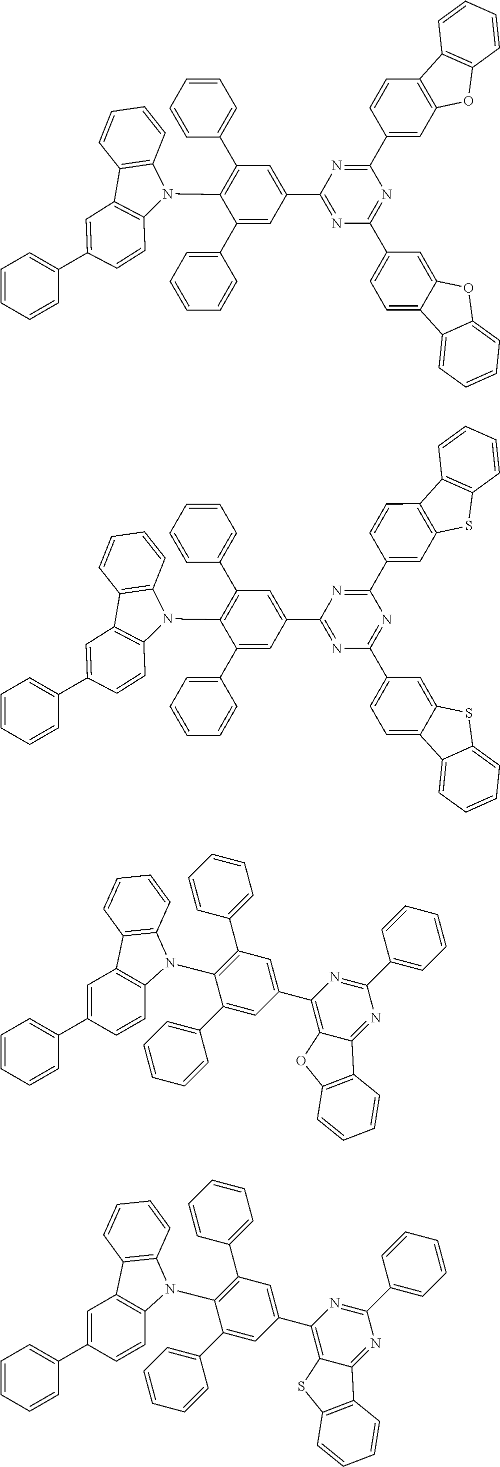

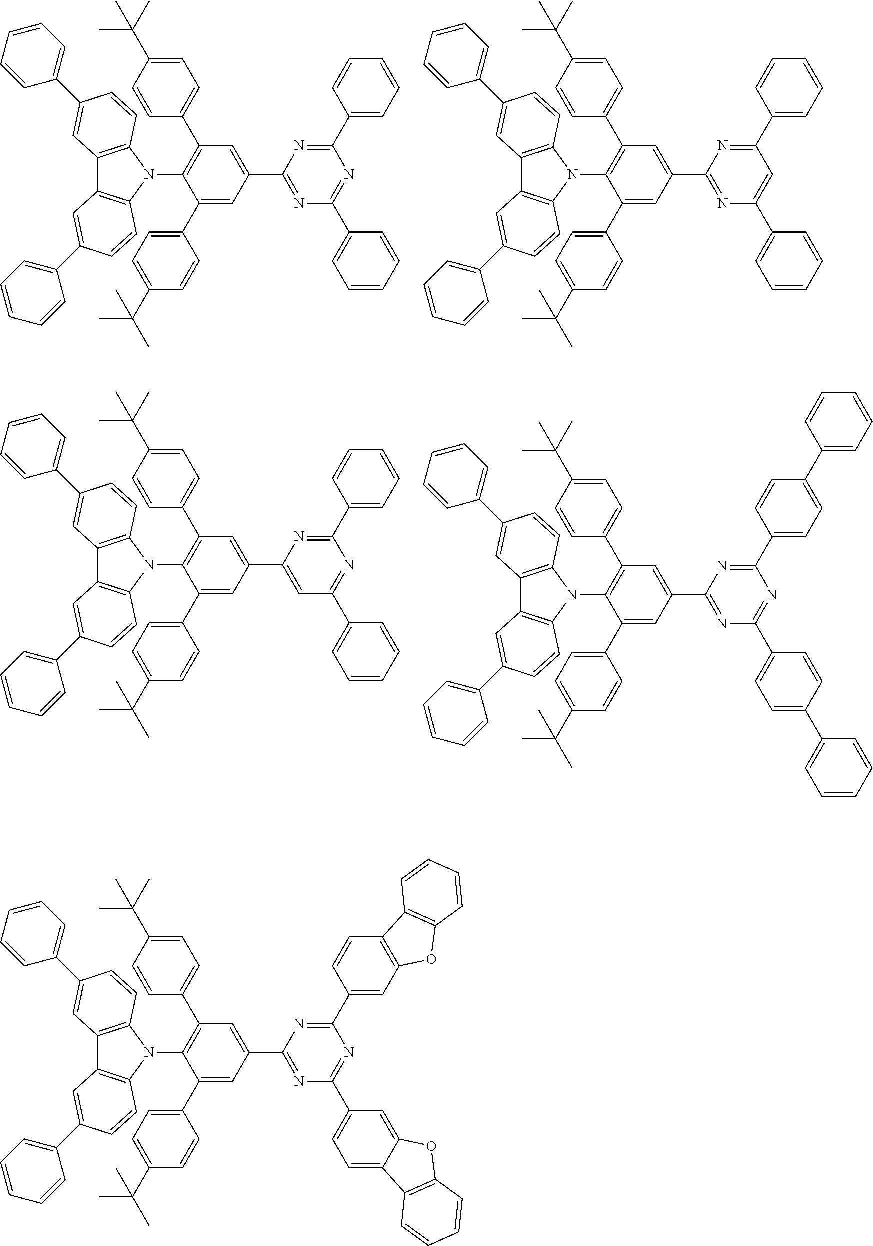

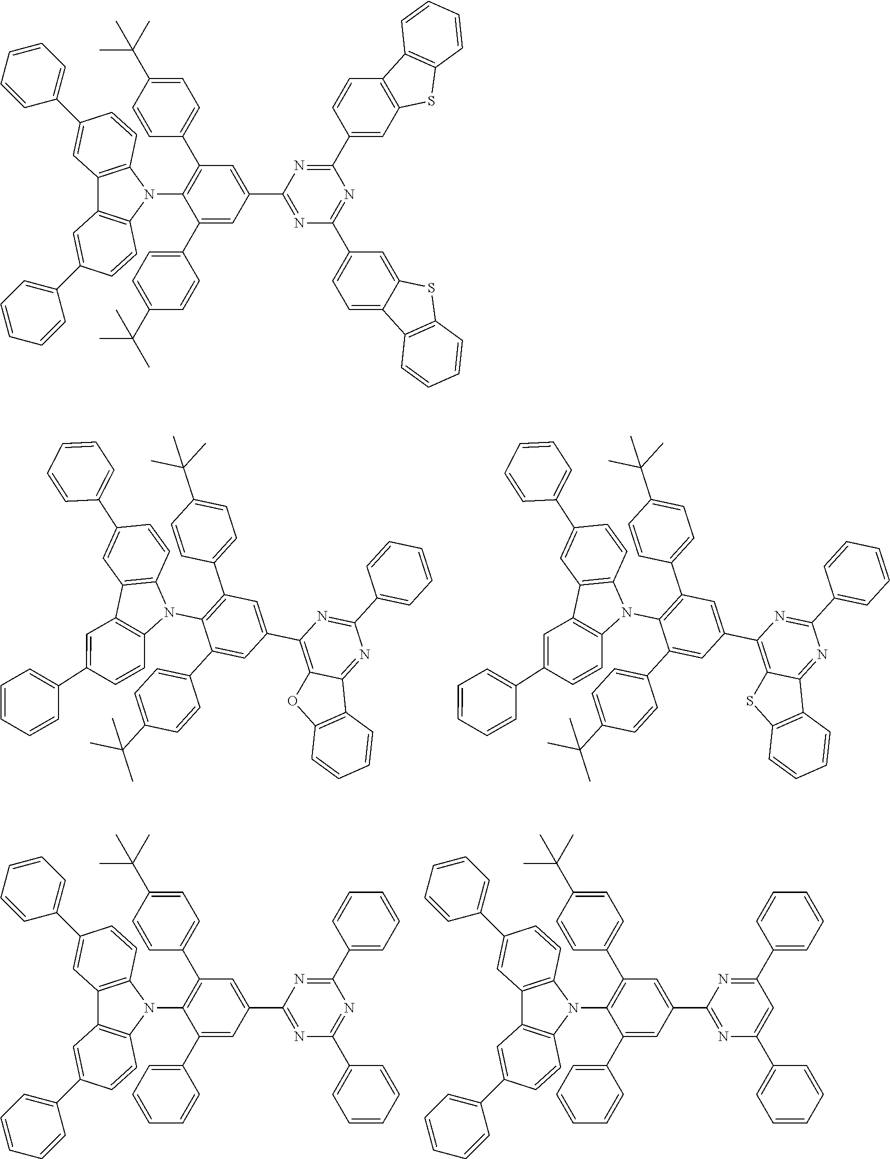

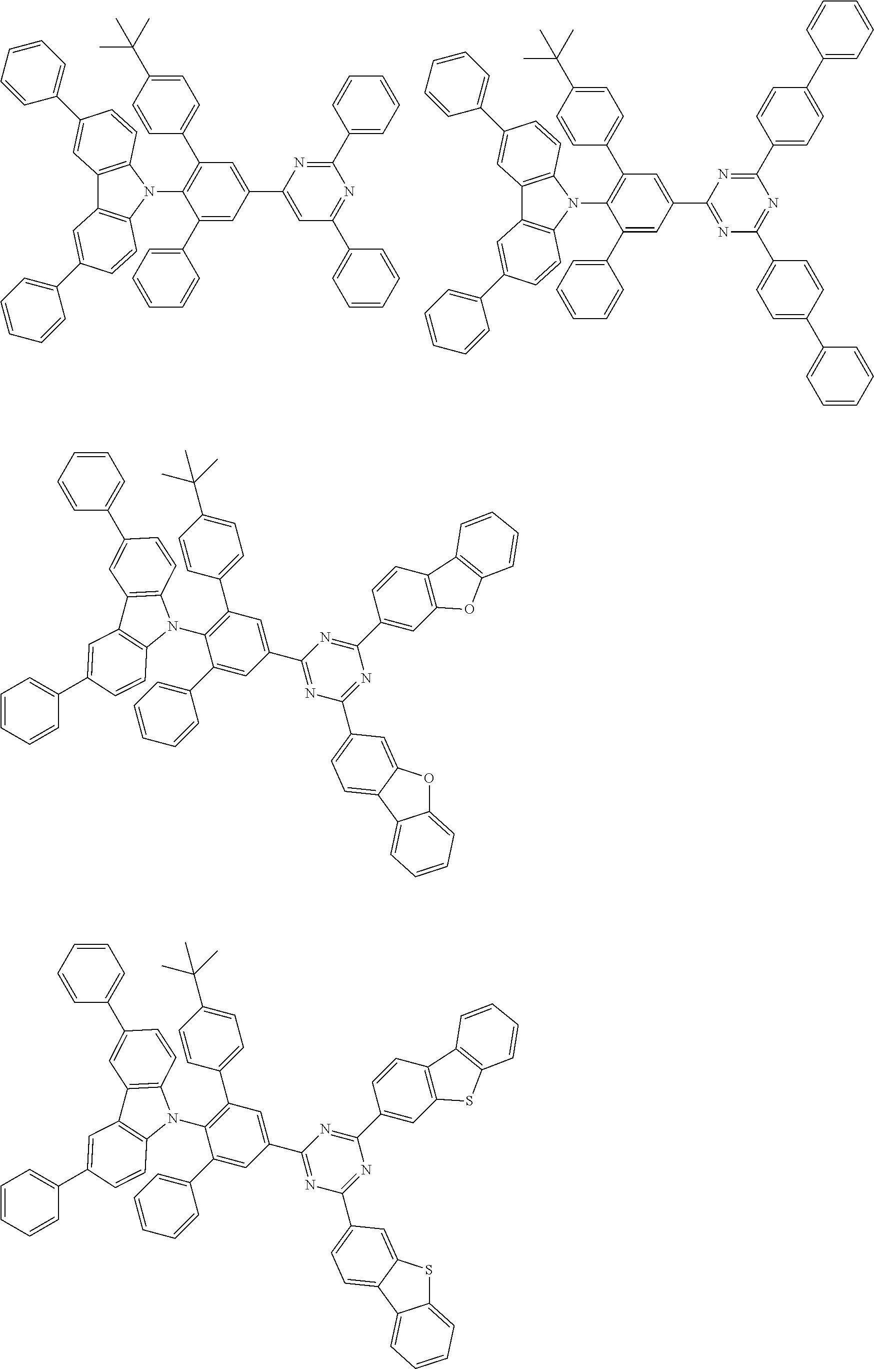

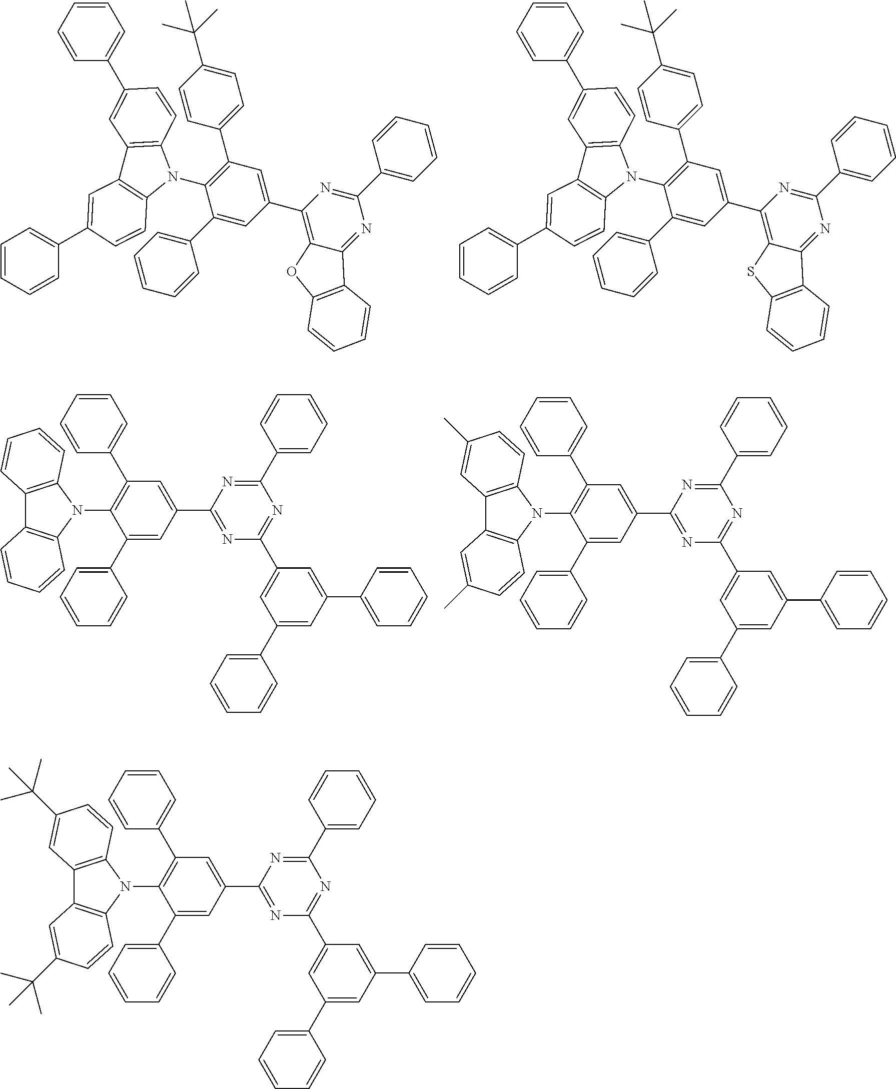

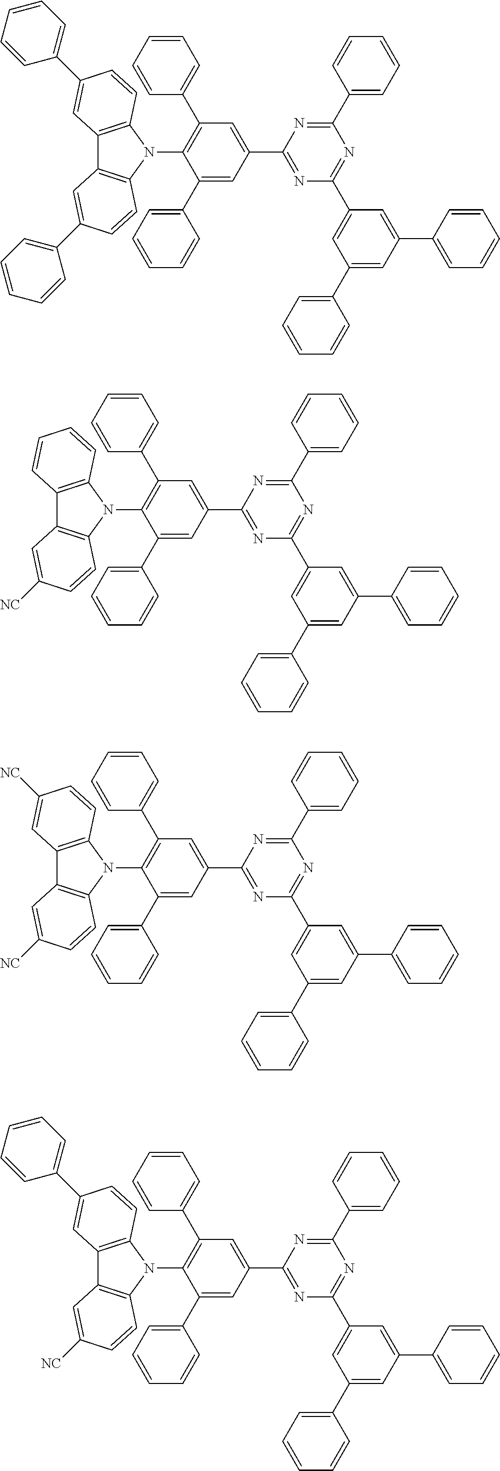

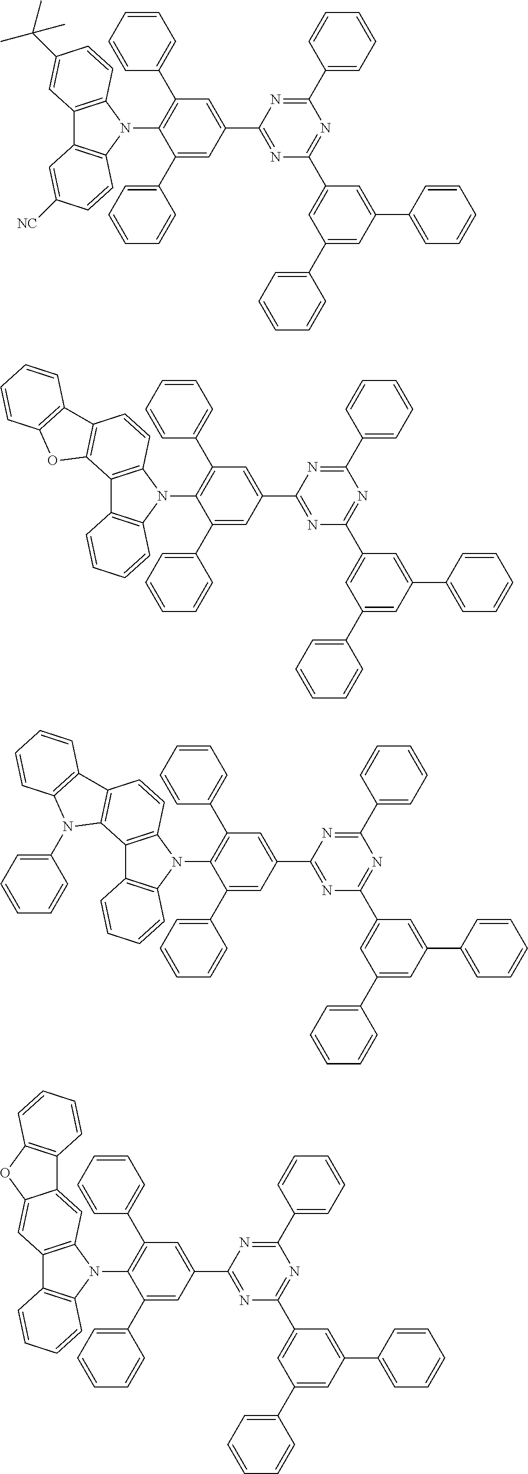 [SMILES: CC(C)(C)c1ccc2c(c1)c1cc(C#N)ccc1n2-c1c(-c2ccccc2)cc(-c2nc(-c3ccccc3)nc(-c3cc(-c4ccccc4)cc(-c4ccccc4)c3)n2)cc1-c1ccccc1 ; c1ccc(-c2cc(-c3ccccc3)cc(-c3nc(-c4ccccc4)nc(-c4cc(-c5ccccc5)c(-n5c6ccccc6c6c7oc8ccccc8c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c5ccc5c7ccccc7n(-c7ccccc7)c56)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6cc7oc8ccccc8c7cc65)c(-c5ccccc5)c4)n3)c2)cc1]

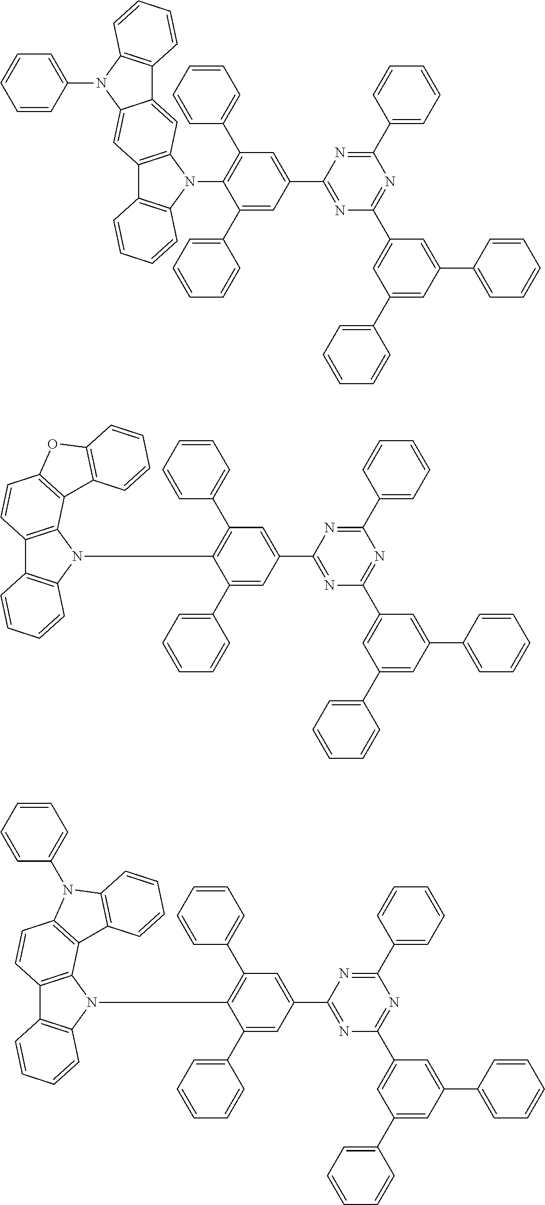 [SMILES: c1ccc(-c2cc(-c3ccccc3)cc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7oc8ccccc8c7c65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)c2)cc1]

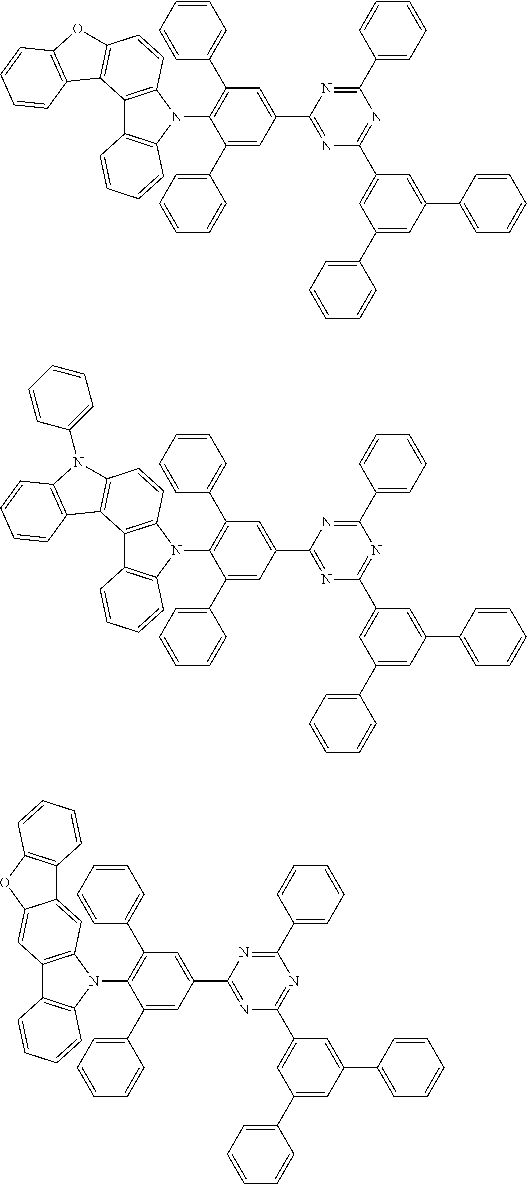 [SMILES: c1ccc(-c2cc(-c3ccccc3)cc(-c3nc(-c4ccccc4)nc(-c4cc(-c5ccccc5)c(-n5c6ccccc6c6c7c(ccc65)oc5ccccc57)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6cc7oc8ccccc8c7cc65)c(-c5ccccc5)c4)n3)c2)cc1]

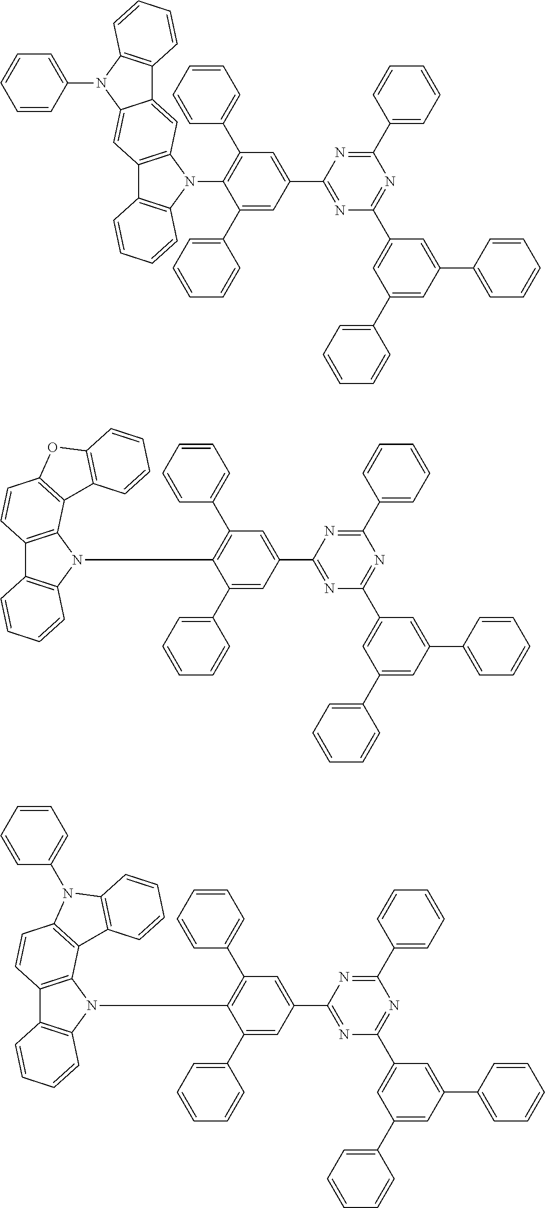 [SMILES: c1ccc(-c2cc(-c3ccccc3)cc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7oc8ccccc8c7c65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)c2)cc1]

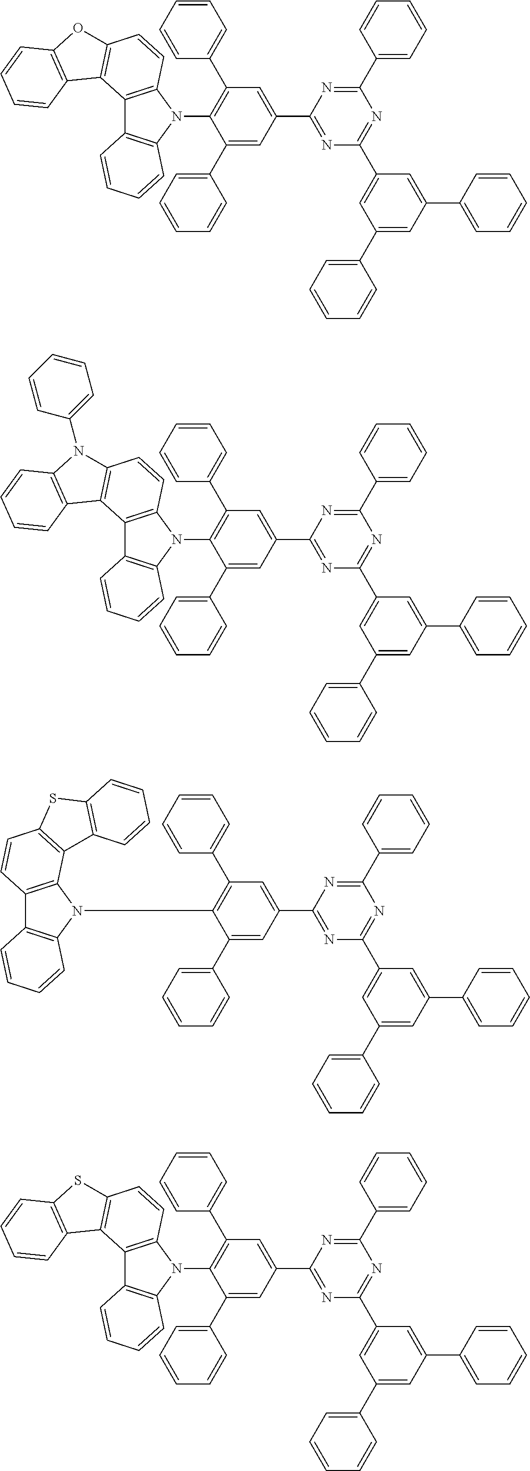

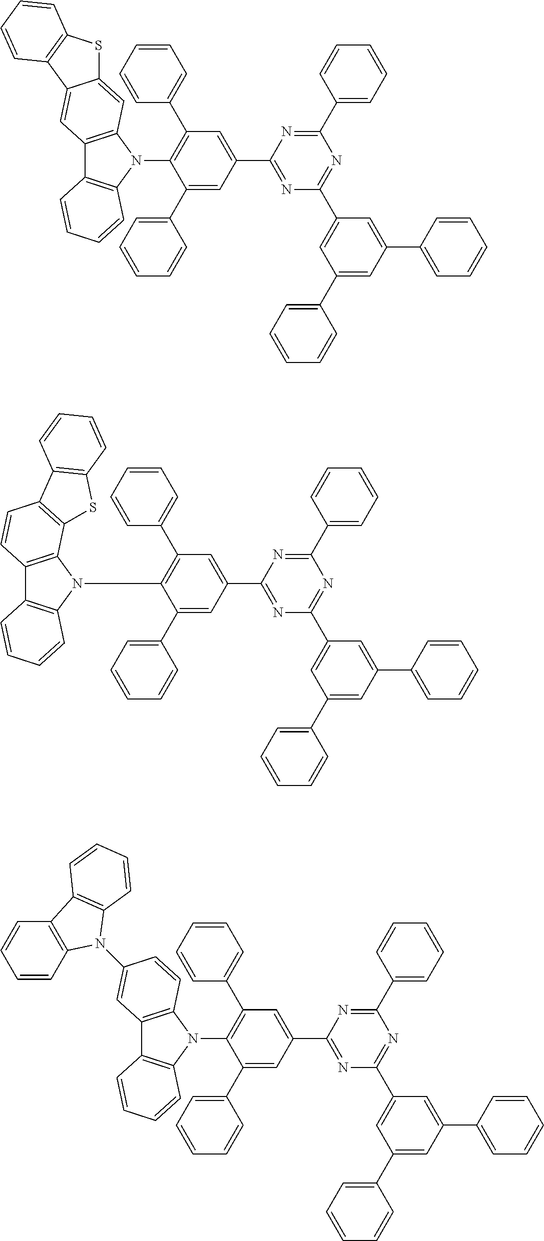 [SMILES: c1ccc(-c2cc(-c3ccccc3)cc(-c3nc(-c4ccccc4)nc(-c4cc(-c5ccccc5)c(-n5c6ccccc6c6cc7c(cc65)sc5ccccc57)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7c8ccccc8sc7c65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6cc(-n7c8ccccc8c8ccccc87)ccc65)c(-c5ccccc5)c4)n3)c2)cc1]

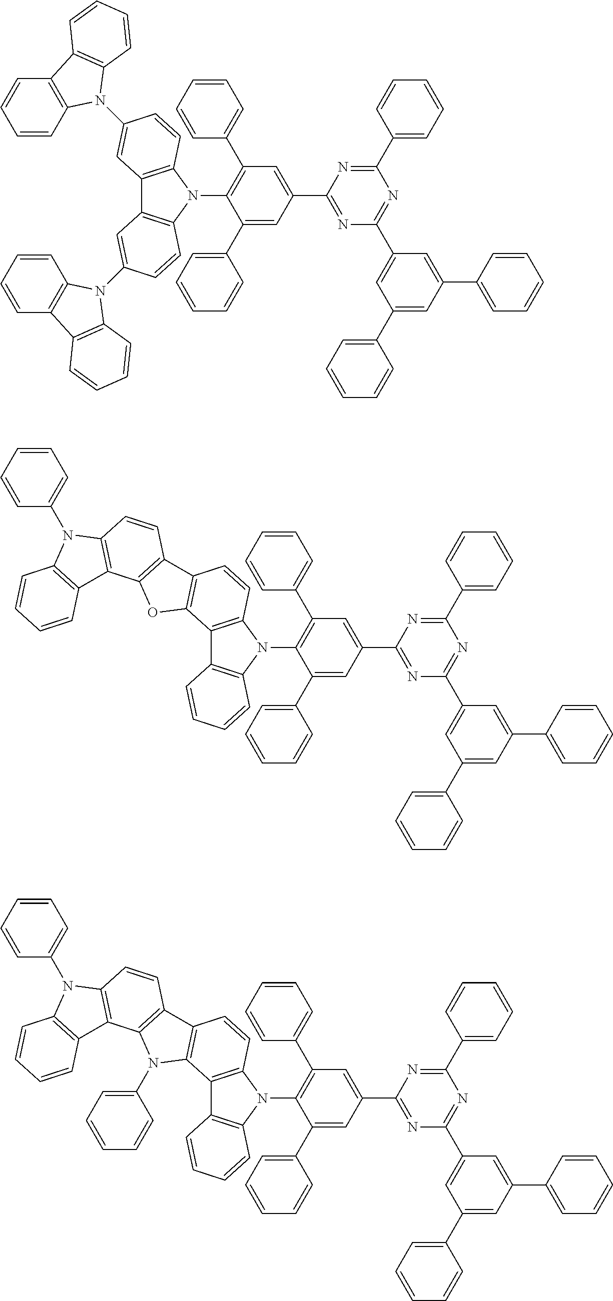 [SMILES: c1ccc(-c2cc(-c3ccccc3)cc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)c2)cc1]

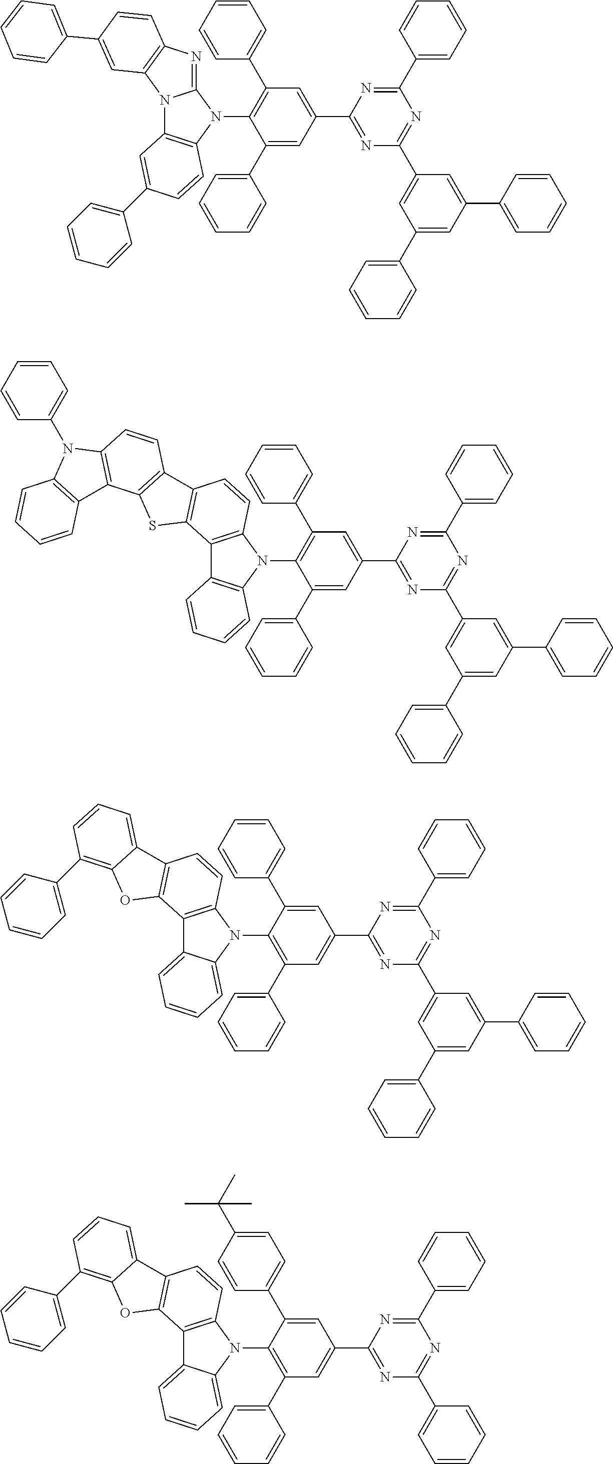

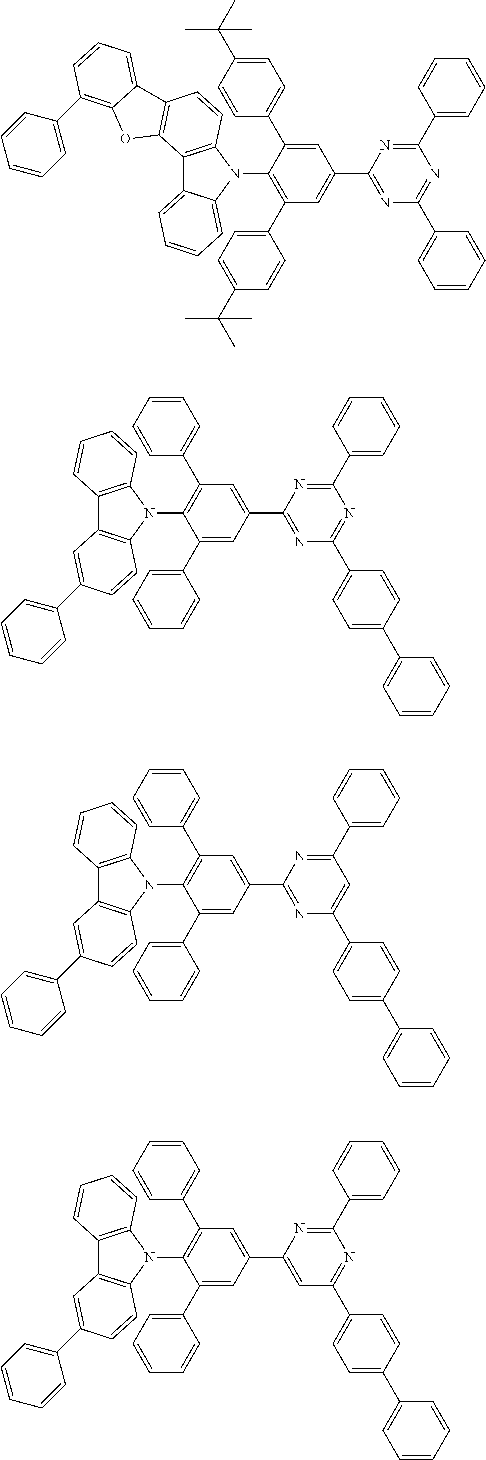 [SMILES: CC(C)(C)c1ccc(-c2cc(-c3nc(-c4ccccc4)nc(-c4ccccc4)n3)cc(-c3ccc(C(C)(C)C)cc3)c2-n2c3ccccc3c3c4oc5c(-c6ccccc6)cccc5c4ccc32)cc1 ; c1ccc(-c2ccc(-c3nc(-c4ccccc4)nc(-c4cc(-c5ccccc5)c(-n5c6ccccc6c6cc(-c7ccccc7)ccc65)c(-c5ccccc5)c4)n3)cc2)cc1 ; c1ccc(-c2ccc(-c3cc(-c4ccccc4)nc(-c4cc(-c5ccccc5)c(-n5c6ccccc6c6cc(-c7ccccc7)ccc65)c(-c5ccccc5)c4)n3)cc2)cc1 ; c1ccc(-c2ccc(-c3cc(-c4cc(-c5ccccc5)c(-n5c6ccccc6c6cc(-c7ccccc7)ccc65)c(-c5ccccc5)c4)nc(-c4ccccc4)n3)cc2)cc1]

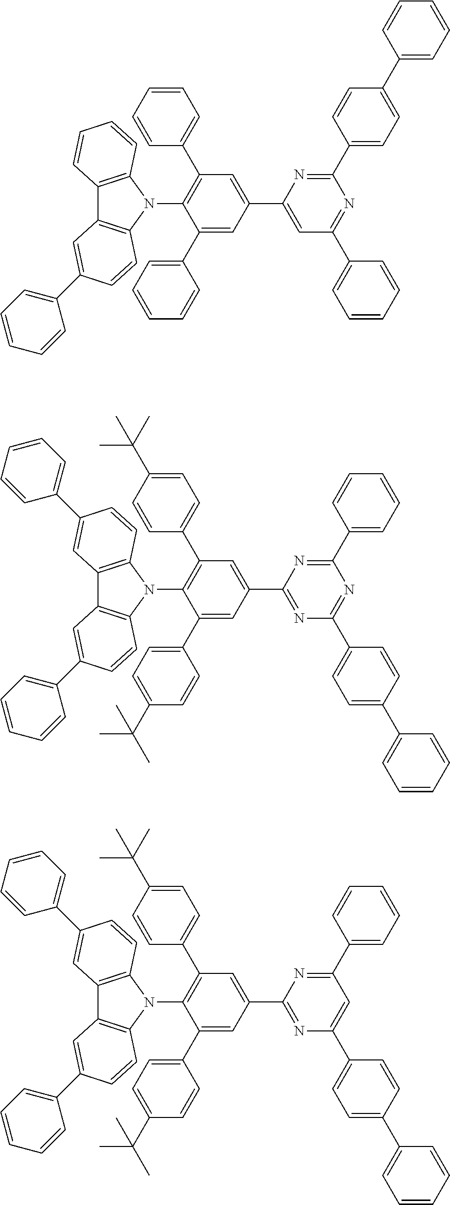 [SMILES: c1ccc(-c2ccc(-c3nc(-c4ccccc4)cc(-c4cc(-c5ccccc5)c(-n5c6ccccc6c6cc(-c7ccccc7)ccc65)c(-c5ccccc5)c4)n3)cc2)cc1 ; CC(C)(C)c1ccc(-c2cc(-c3nc(-c4ccccc4)nc(-c4ccc(-c5ccccc5)cc4)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)cc(-c4ccc(-c5ccccc5)cc4)n3)cc(-c3ccc(C(C)(C)C)cc3)c2-n2c3ccc(-c4ccccc4)cc3c3cc(-c4ccccc4)ccc32)cc1]

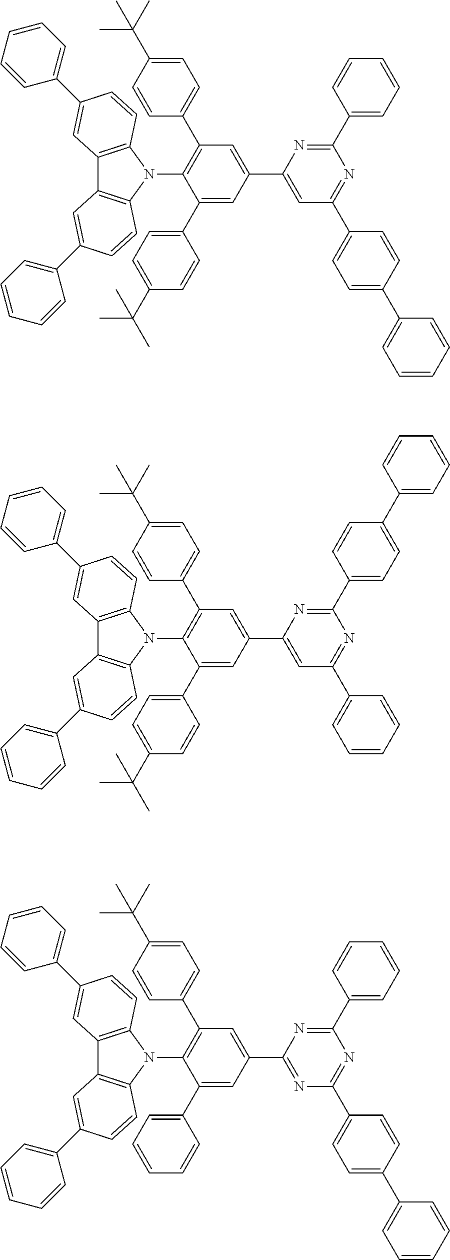 [SMILES: CC(C)(C)c1ccc(-c2cc(-c3cc(-c4ccc(-c5ccccc5)cc4)nc(-c4ccccc4)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3cc(-c4ccccc4)nc(-c4ccc(-c5ccccc5)cc4)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)nc(-c4ccc(-c5ccccc5)cc4)n3)cc(-c3ccccc3)c2-n2c3ccc(-c4ccccc4)cc3c3cc(-c4ccccc4)ccc32)cc1]

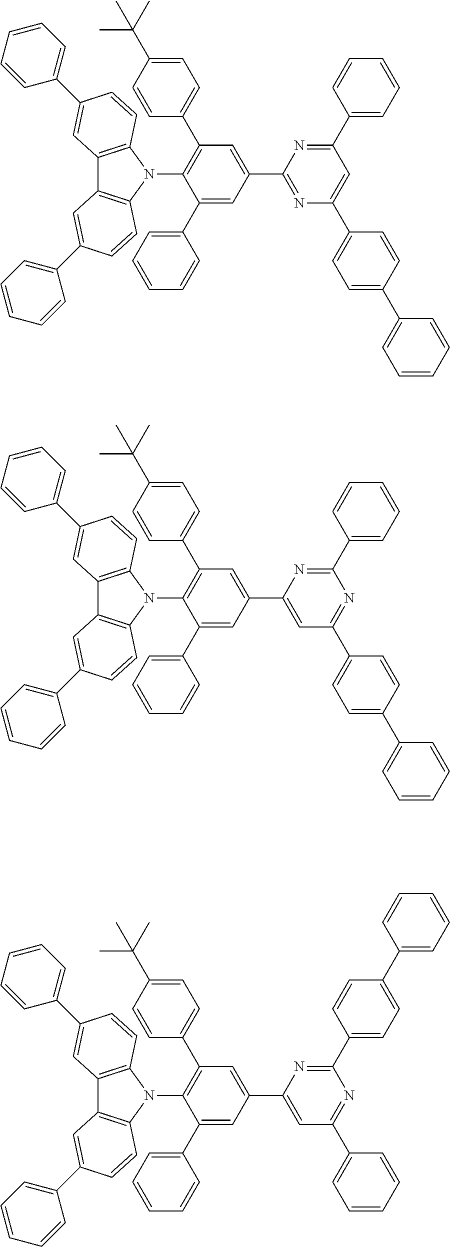 [SMILES: CC(C)(C)c1ccc(-c2cc(-c3nc(-c4ccccc4)cc(-c4ccc(-c5ccccc5)cc4)n3)cc(-c3ccccc3)c2-n2c3ccc(-c4ccccc4)cc3c3cc(-c4ccccc4)ccc32)cc1 ; CC(C)(C)c1ccc(-c2cc(-c3cc(-c4ccc(-c5ccccc5)cc4)nc(-c4ccccc4)n3)cc(-c3ccccc3)c2-n2c3ccc(-c4ccccc4)cc3c3cc(-c4ccccc4)ccc32)cc1 ; CC(C)(C)c1ccc(-c2cc(-c3cc(-c4ccccc4)nc(-c4ccc(-c5ccccc5)cc4)n3)cc(-c3ccccc3)c2-n2c3ccc(-c4ccccc4)cc3c3cc(-c4ccccc4)ccc32)cc1]

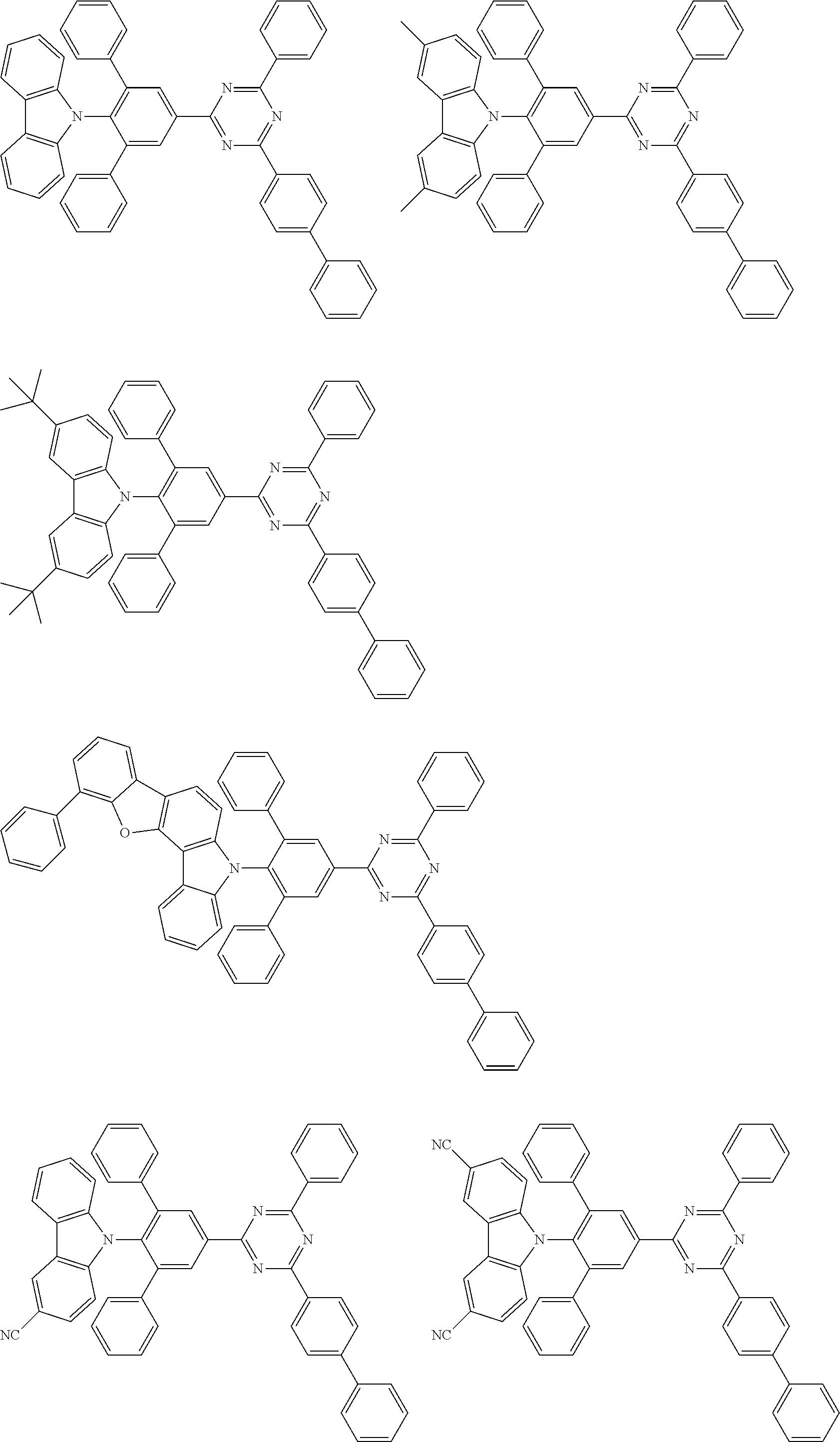

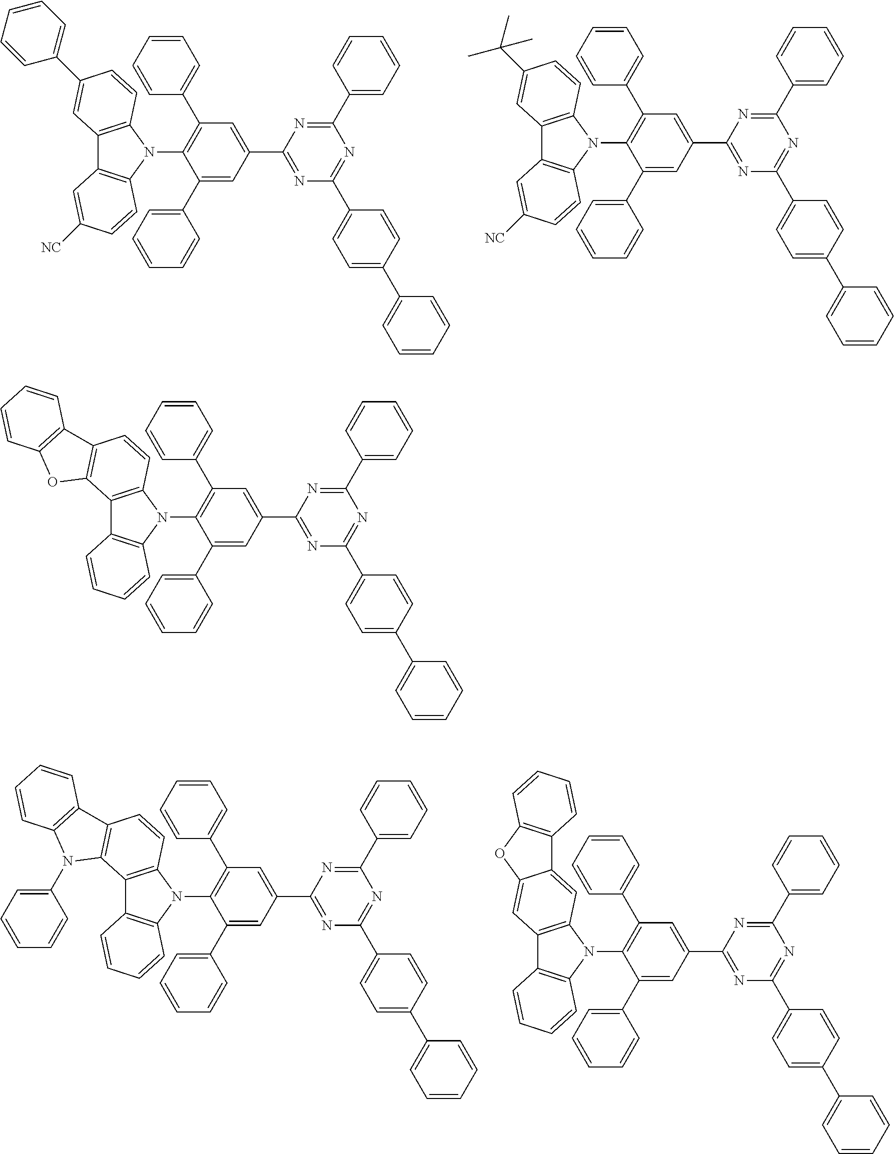

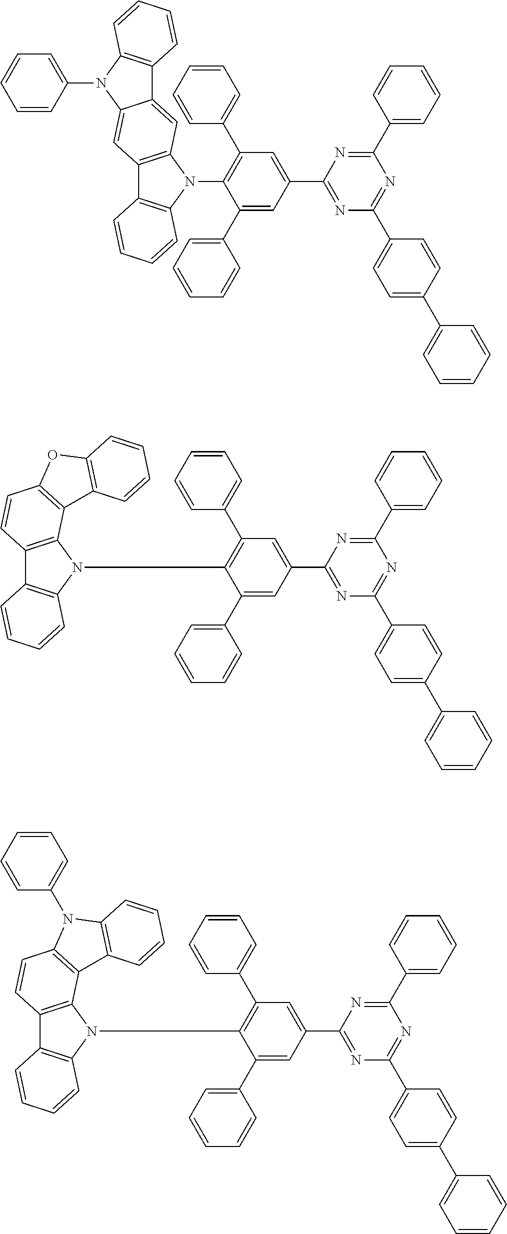 [SMILES: c1ccc(-c2ccc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7oc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)cc2)cc1]

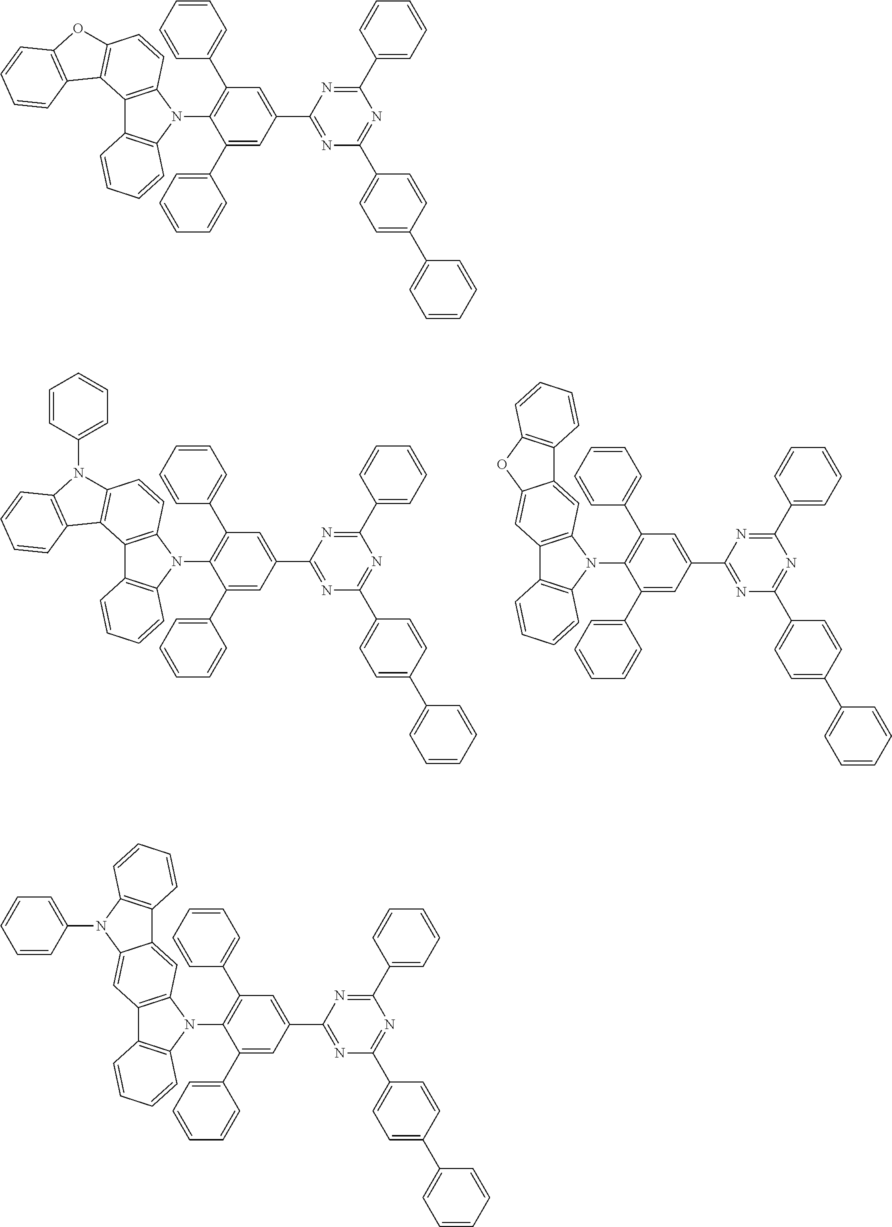 [SMILES: c1ccc(-c2ccc(-c3nc(-c4ccccc4)nc(-c4cc(-c5ccccc5)c(-n5c6ccccc6c6c7c(ccc65)oc5ccccc57)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7oc8ccccc8c7cc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)cc2)cc1]

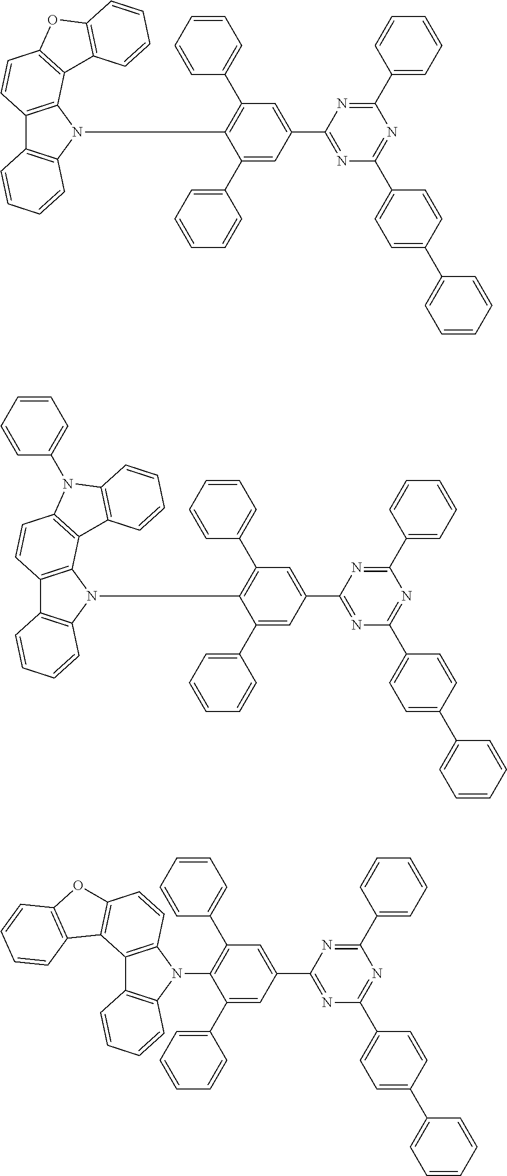 [SMILES: c1ccc(-c2ccc(-c3nc(-c4ccccc4)nc(-c4cc(-c5ccccc5)c(-n5c6ccccc6c6ccc7oc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)oc5ccccc57)c(-c5ccccc5)c4)n3)cc2)cc1]

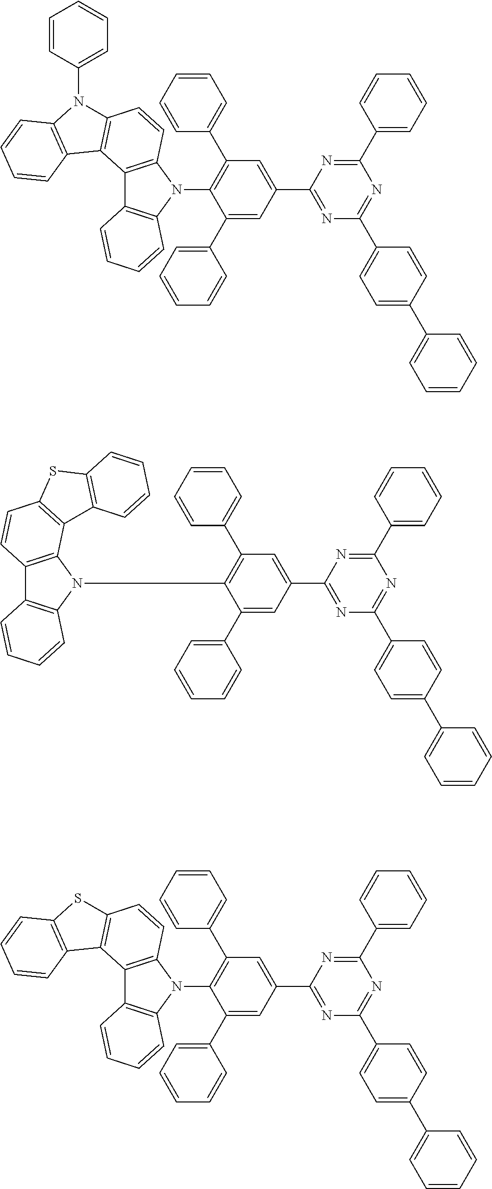 [SMILES: c1ccc(-c2ccc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7sc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)sc5ccccc57)c(-c5ccccc5)c4)n3)cc2)cc1]

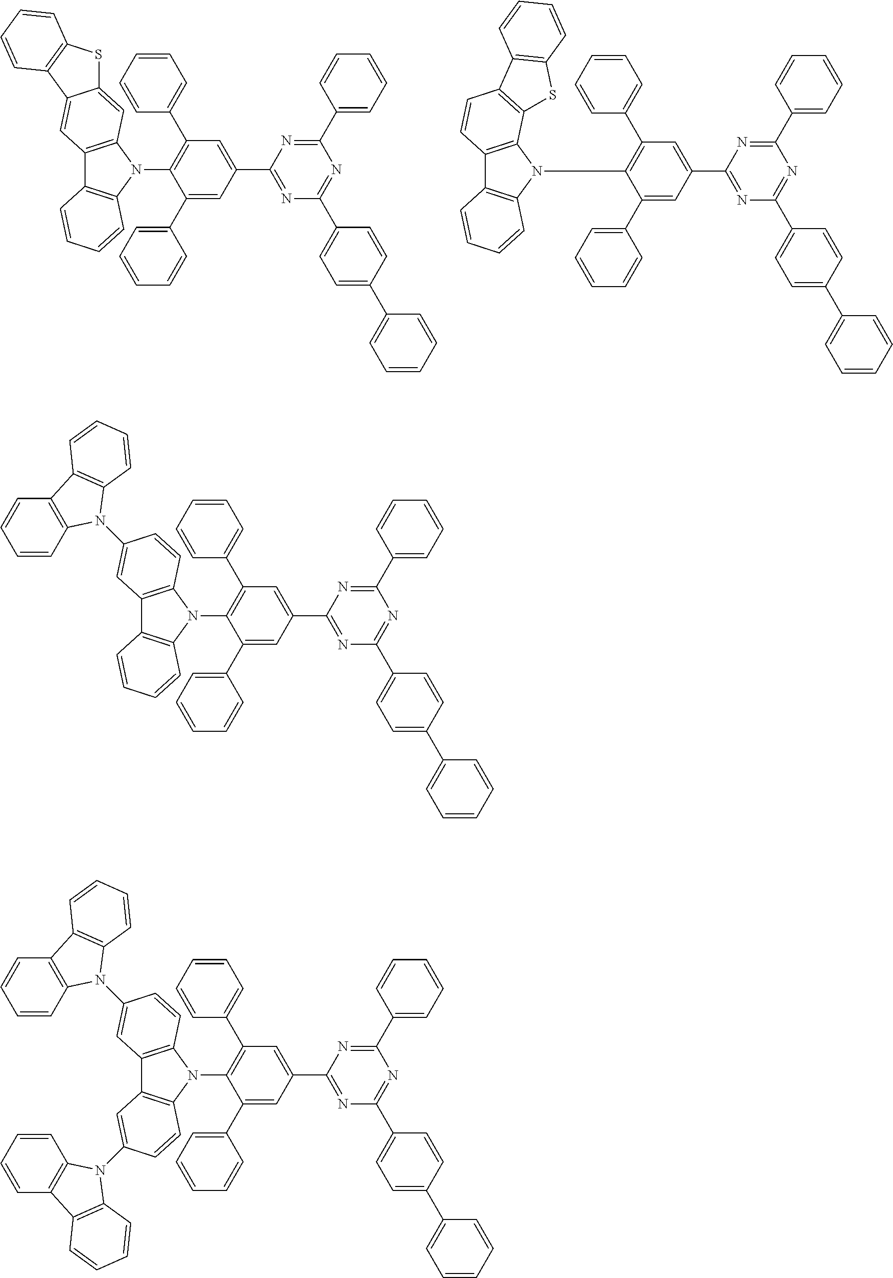 [SMILES: c1ccc(-c2ccc(-c3nc(-c4ccccc4)nc(-c4cc(-c5ccccc5)c(-n5c6ccccc6c6cc7c(cc65)sc5ccccc57)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7c8ccccc8sc7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc(-n7c8ccccc8c8ccccc87)ccc65)c(-c5ccccc5)c4)n3)cc2)cc1 ; c1ccc(-c2ccc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1]

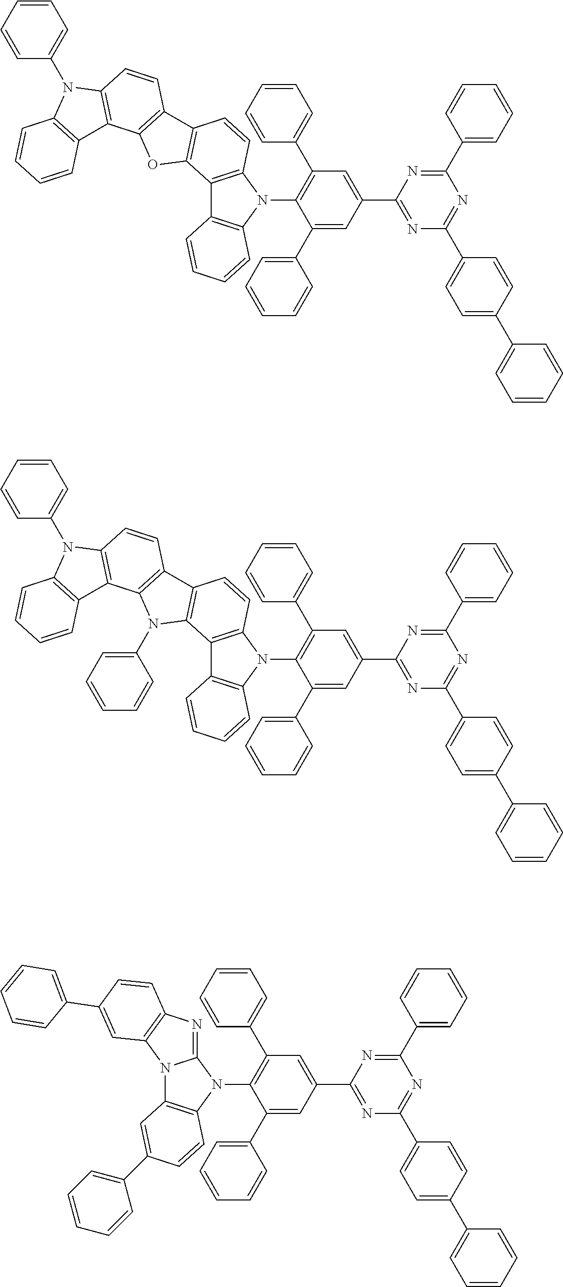 [SMILES: c1ccc(-c2ccc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1 ; c1ccc(-c2ccc(-c3nc(-c4ccccc4)nc(-c4cc(-c5ccccc5)c(-n5c6ccc(-c7ccccc7)cc6n6c7cc(-c8ccccc8)ccc7nc56)c(-c5ccccc5)c4)n3)cc2)cc1]

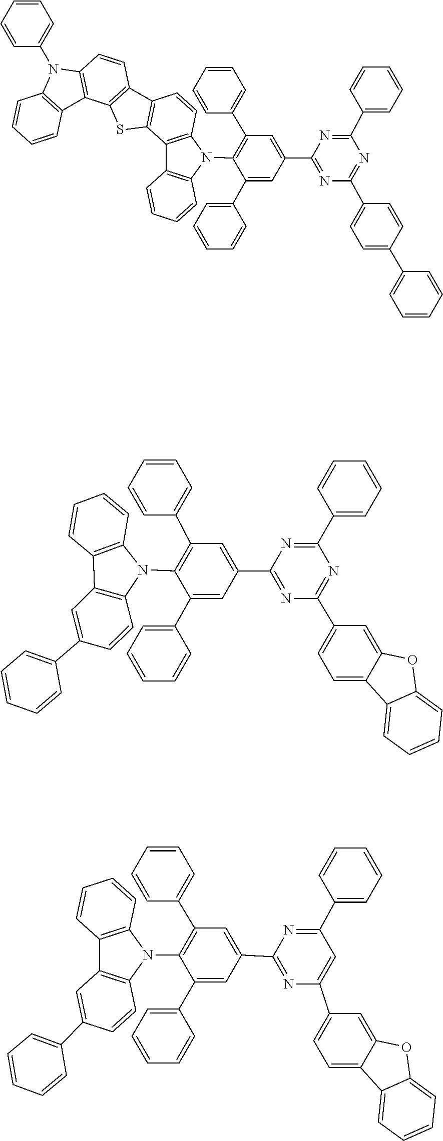 [SMILES: c1ccc(-c2ccc(-c3nc(-c4ccccc4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1 ; c1ccc(-c2ccc3c(c2)c2ccccc2n3-c2c(-c3ccccc3)cc(-c3nc(-c4ccccc4)nc(-c4ccc5c(c4)oc4ccccc45)n3)cc2-c2ccccc2)cc1 ; c1ccc(-c2ccc3c(c2)c2ccccc2n3-c2c(-c3ccccc3)cc(-c3nc(-c4ccccc4)cc(-c4ccc5c(c4)oc4ccccc45)n3)cc2-c2ccccc2)cc1]

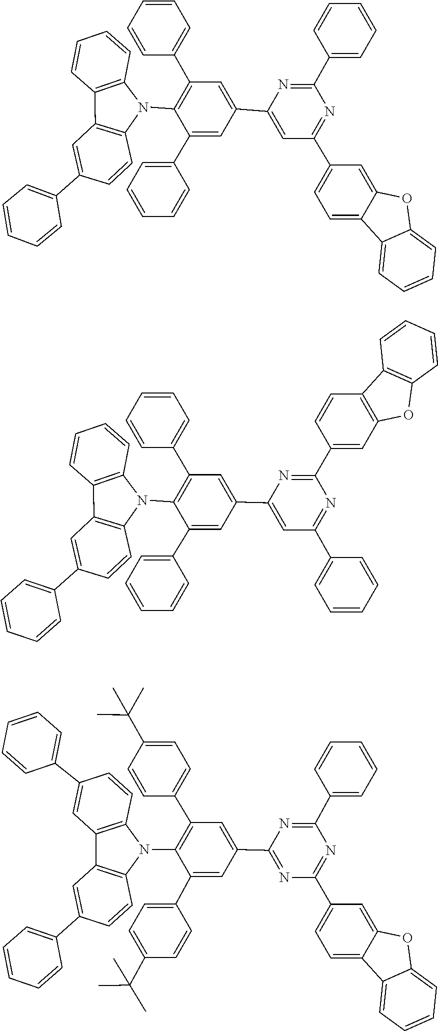

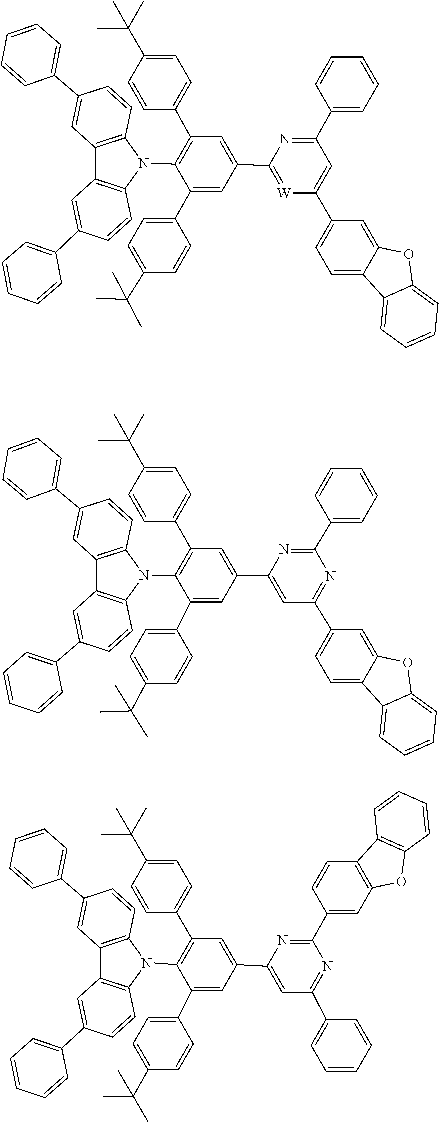

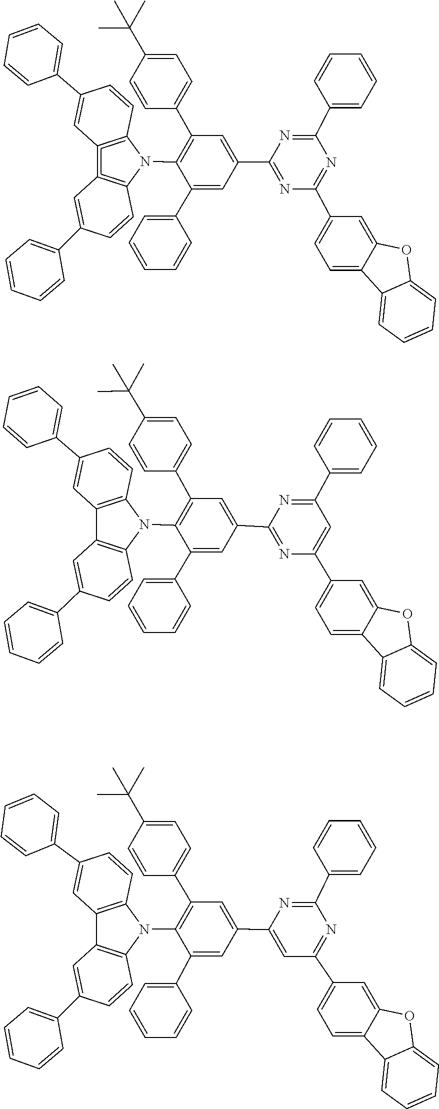

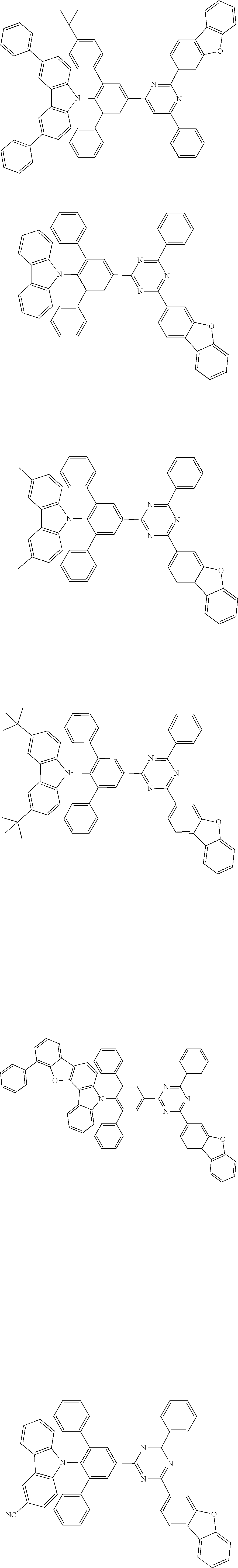

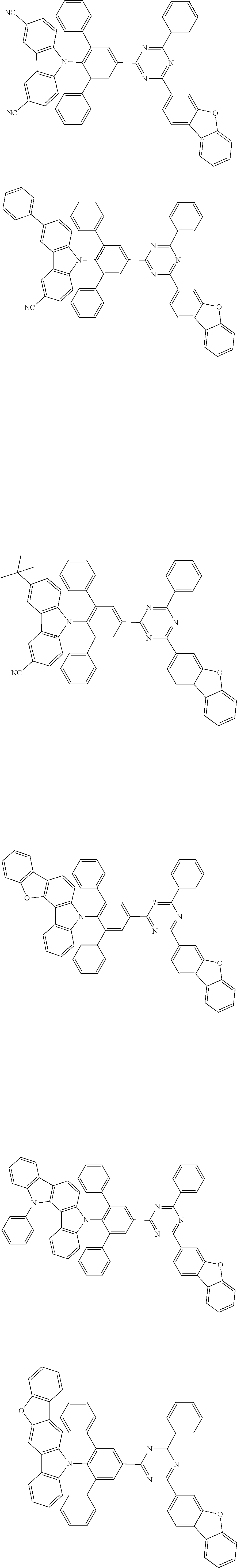

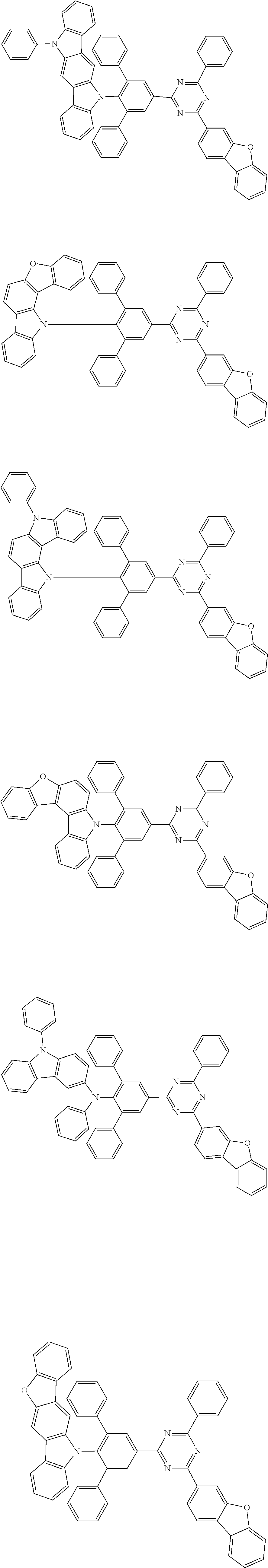

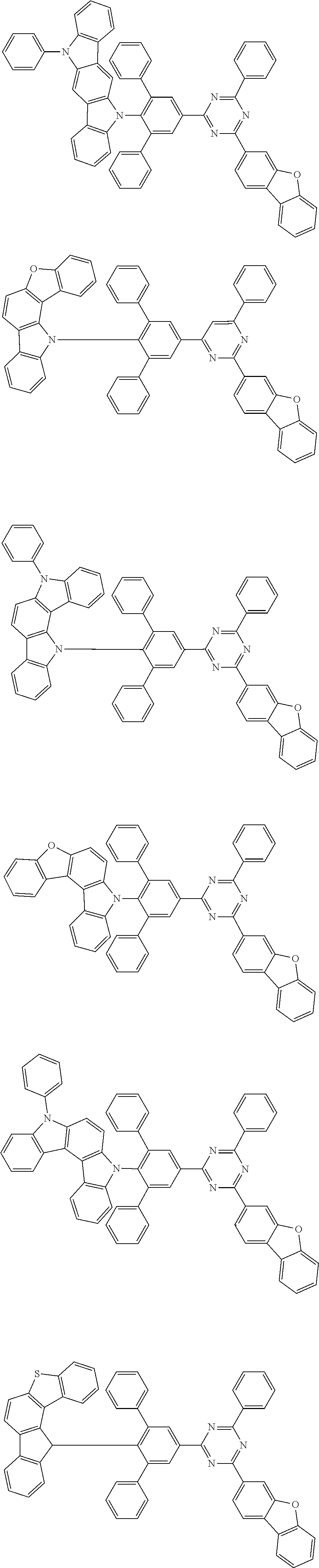

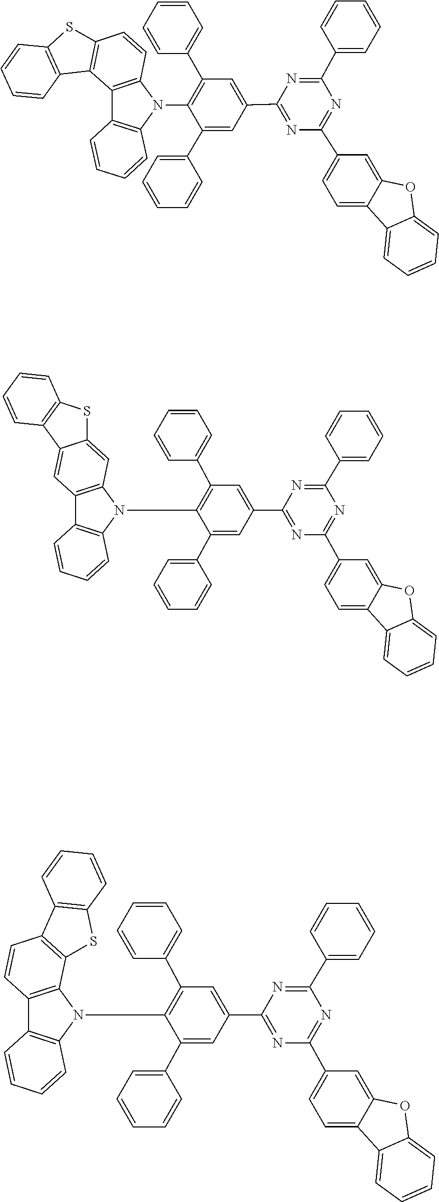 [SMILES: c1ccc(-c2nc(-c3cc(-c4ccccc4)c(-n4c5ccccc5c5c6c(ccc54)sc4ccccc46)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6c(cc54)sc4ccccc46)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6c7ccccc7sc6c54)c(-c4ccccc4)c3)nc(-c3ccc4c(c3)oc3ccccc34)n2)cc1]

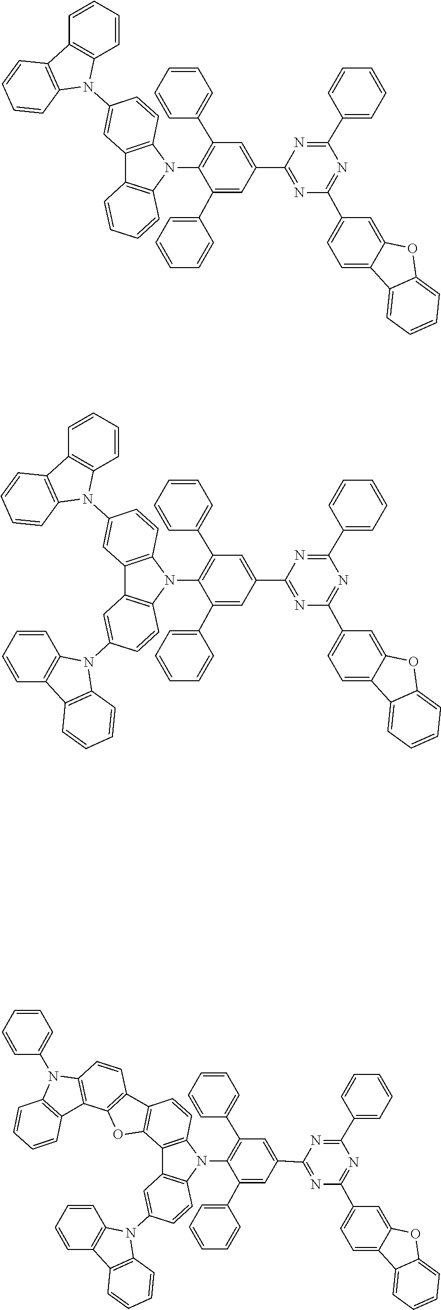 [SMILES: c1ccc(-c2nc(-c3cc(-c4ccccc4)c(-n4c5ccccc5c5cc(-n6c7ccccc7c7ccccc76)ccc54)c(-c4ccccc4)c3)nc(-c3ccc4c(c3)oc3ccccc34)n2)cc1 ; c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)nc(-c3ccc4c(c3)oc3ccccc34)n2)cc1 ; c1ccc(-c2nc(-c3cc(-c4ccccc4)c(-n4c5ccc(-n6c7ccccc7c7ccccc76)cc5c5c6oc7c(ccc8c7c7ccccc7n8-c7ccccc7)c6ccc54)c(-c4ccccc4)c3)nc(-c3ccc4c(c3)oc3ccccc34)n2)cc1]

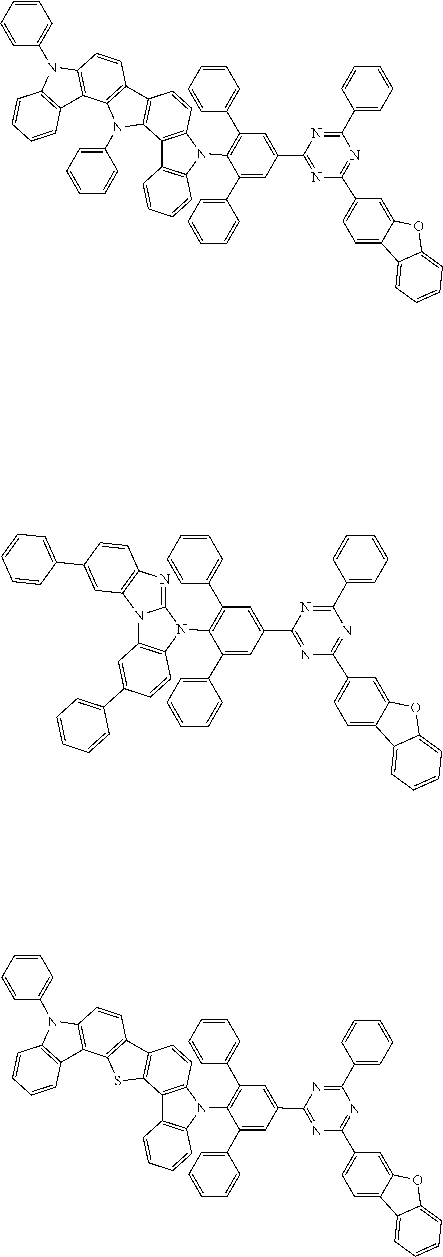 [SMILES: c1ccc(-c2nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)nc(-c3ccc4c(c3)oc3ccccc34)n2)cc1 ; c1ccc(-c2ccc3nc4n(-c5c(-c6ccccc6)cc(-c6nc(-c7ccccc7)nc(-c7ccc8c(c7)oc7ccccc78)n6)cc5-c5ccccc5)c5ccc(-c6ccccc6)cc5n4c3c2)cc1 ; c1ccc(-c2nc(-c3cc(-c4ccccc4)c(-n4c5ccccc5c5c6sc7c(ccc8c7c7ccccc7n8-c7ccccc7)c6ccc54)c(-c4ccccc4)c3)nc(-c3ccc4c(c3)oc3ccccc34)n2)cc1]

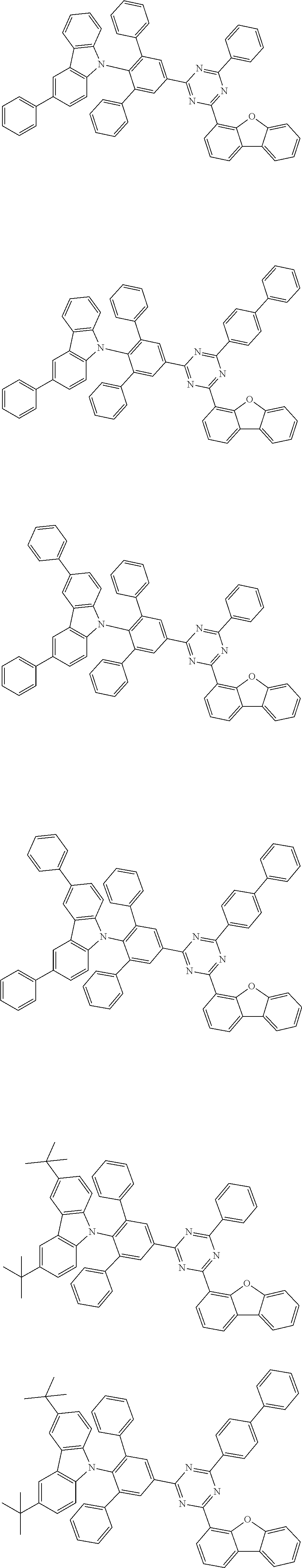

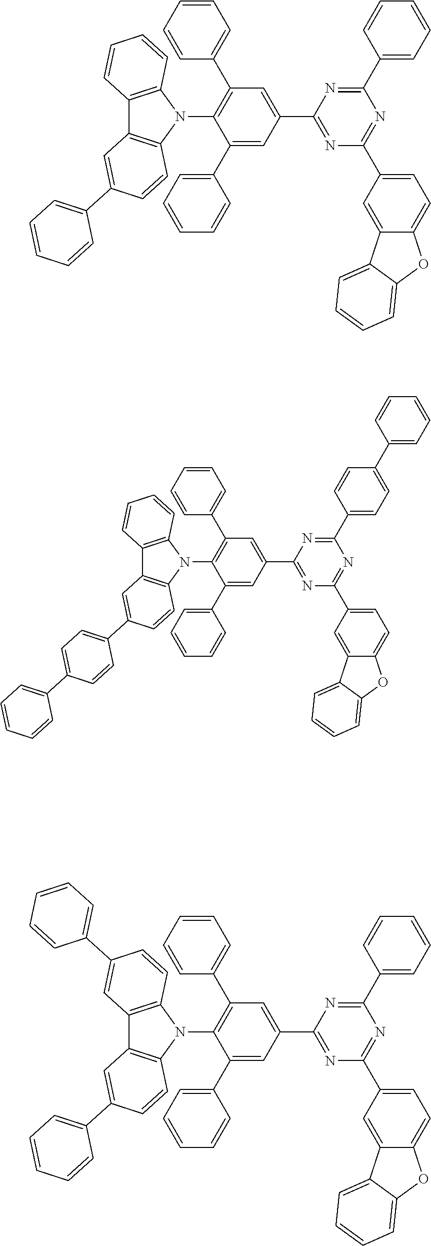 [SMILES: c1ccc(-c2ccc3c(c2)c2ccccc2n3-c2c(-c3ccccc3)cc(-c3nc(-c4ccccc4)nc(-c4ccc5oc6ccccc6c5c4)n3)cc2-c2ccccc2)cc1 ; c1ccc(-c2ccc(-c3ccc4c(c3)c3ccccc3n4-c3c(-c4ccccc4)cc(-c4nc(-c5ccc(-c6ccccc6)cc5)nc(-c5ccc6oc7ccccc7c6c5)n4)cc3-c3ccccc3)cc2)cc1 ; c1ccc(-c2ccc3c(c2)c2cc(-c4ccccc4)ccc2n3-c2c(-c3ccccc3)cc(-c3nc(-c4ccccc4)nc(-c4ccc5oc6ccccc6c5c4)n3)cc2-c2ccccc2)cc1]

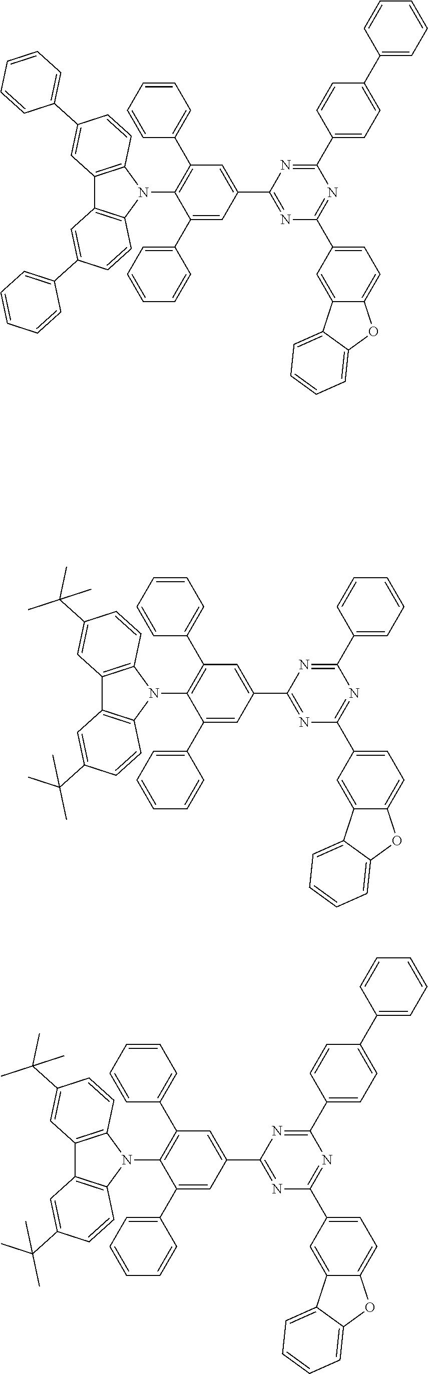 [SMILES: c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4ccc5oc6ccccc6c5c4)n3)cc2)cc1 ; CC(C)(C)c1ccc2c(c1)c1cc(C(C)(C)C)ccc1n2-c1c(-c2ccccc2)cc(-c2nc(-c3ccccc3)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3ccc4oc5ccccc5c4c3)n2)cc1-c1ccccc1]

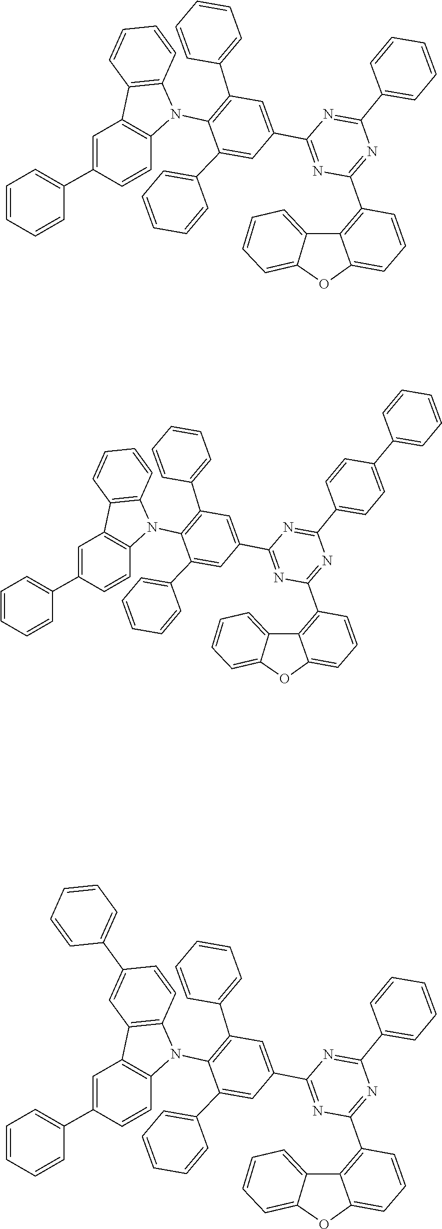 [SMILES: c1ccc(-c2ccc3c(c2)c2ccccc2n3-c2c(-c3ccccc3)cc(-c3nc(-c4ccccc4)nc(-c4cccc5oc6ccccc6c45)n3)cc2-c2ccccc2)cc1 ; c1ccc(-c2ccc(-c3nc(-c4cc(-c5ccccc5)c(-n5c6ccccc6c6cc(-c7ccccc7)ccc65)c(-c5ccccc5)c4)nc(-c4cccc5oc6ccccc6c45)n3)cc2)cc1 ; c1ccc(-c2ccc3c(c2)c2cc(-c4ccccc4)ccc2n3-c2c(-c3ccccc3)cc(-c3nc(-c4ccccc4)nc(-c4cccc5oc6ccccc6c45)n3)cc2-c2ccccc2)cc1]

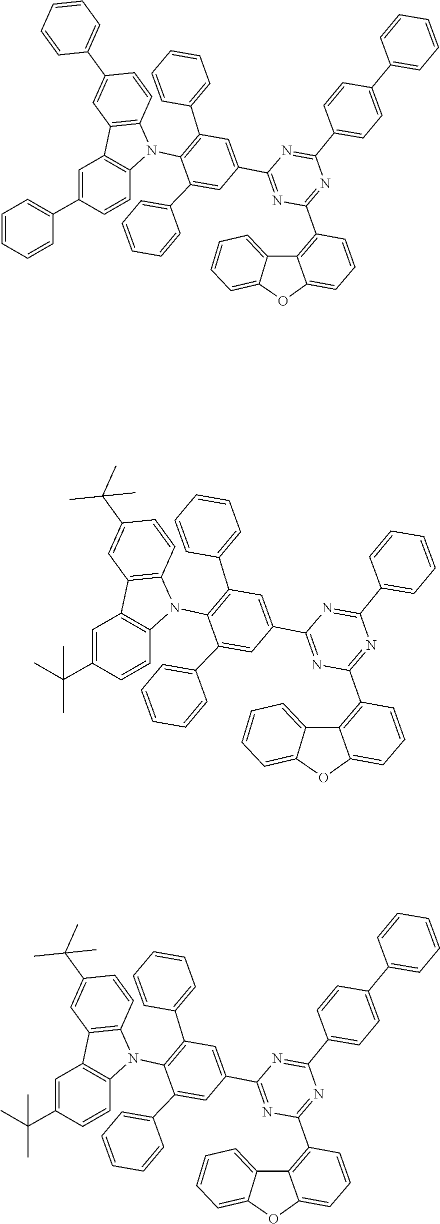 [SMILES: c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4cccc5oc6ccccc6c45)n3)cc2)cc1 ; CC(C)(C)c1ccc2c(c1)c1cc(C(C)(C)C)ccc1n2-c1c(-c2ccccc2)cc(-c2nc(-c3ccccc3)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cccc4oc5ccccc5c34)n2)cc1-c1ccccc1]

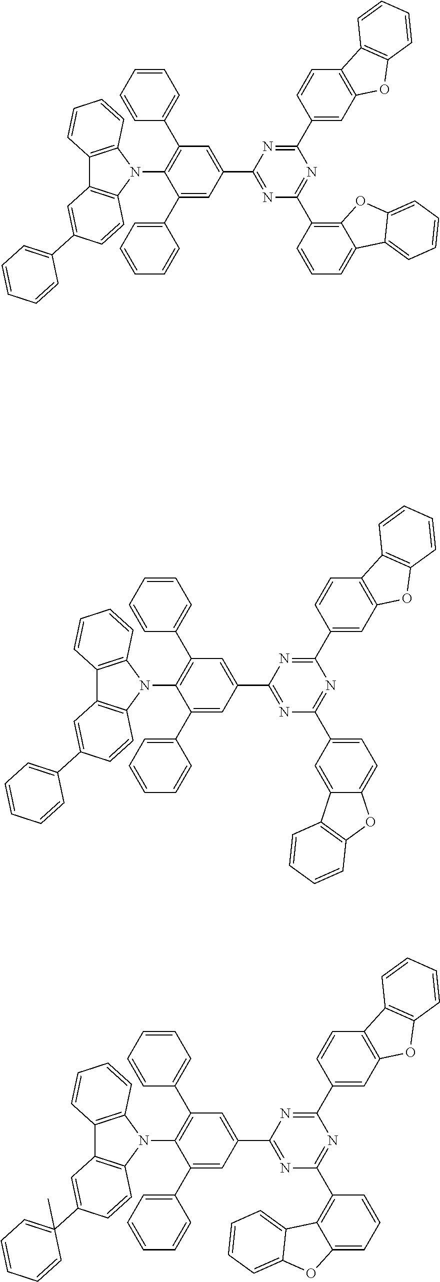

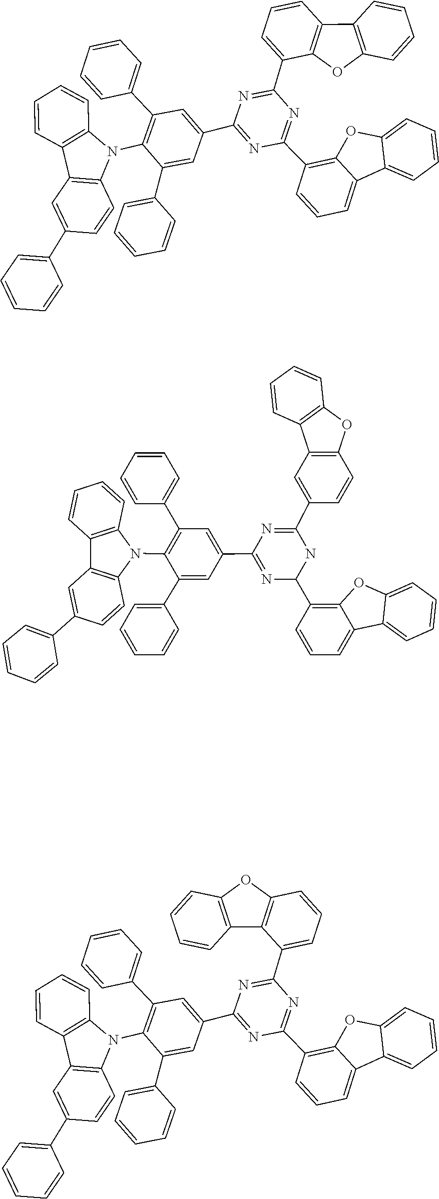

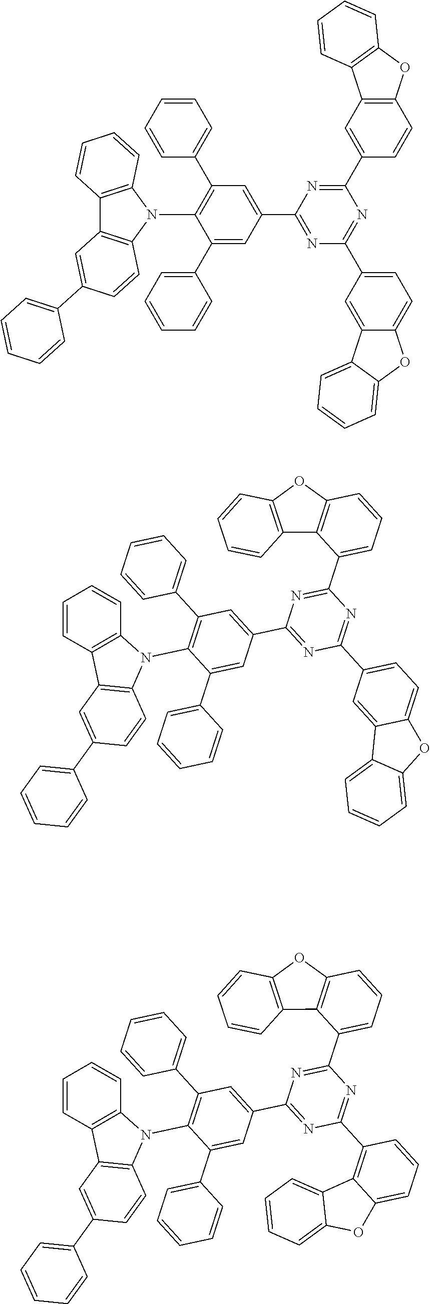 [SMILES: c1ccc(-c2ccc3c(c2)c2ccccc2n3-c2c(-c3ccccc3)cc(-c3nc(-c4ccc5oc6ccccc6c5c4)nc(-c4ccc5oc6ccccc6c5c4)n3)cc2-c2ccccc2)cc1 ; c1ccc(-c2ccc3c(c2)c2ccccc2n3-c2c(-c3ccccc3)cc(-c3nc(-c4ccc5oc6ccccc6c5c4)nc(-c4cccc5oc6ccccc6c45)n3)cc2-c2ccccc2)cc1 ; c1ccc(-c2ccc3c(c2)c2ccccc2n3-c2c(-c3ccccc3)cc(-c3nc(-c4cccc5oc6ccccc6c45)nc(-c4cccc5oc6ccccc6c45)n3)cc2-c2ccccc2)cc1]

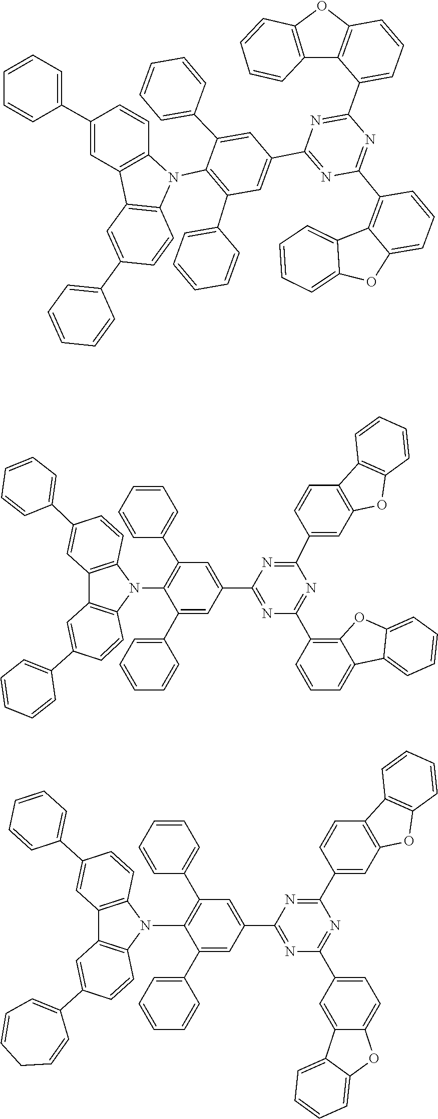 [SMILES: c1ccc(-c2ccc3c(c2)c2cc(-c4ccccc4)ccc2n3-c2c(-c3ccccc3)cc(-c3nc(-c4cccc5oc6ccccc6c45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5c(c4)oc4ccccc45)nc(-c4cccc5c4oc4ccccc45)n3)cc2-c2ccccc2)cc1 ; C1=CCC=CC(c2ccc3c(c2)c2cc(-c4ccccc4)ccc2n3-c2c(-c3ccccc3)cc(-c3nc(-c4ccc5c(c4)oc4ccccc45)nc(-c4ccc5oc6ccccc6c5c4)n3)cc2-c2ccccc2)=C1]

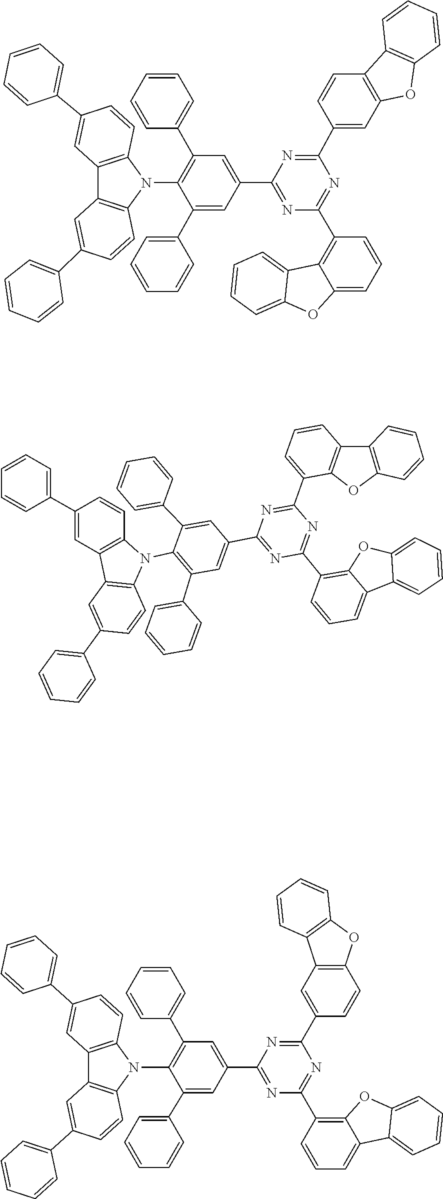 [SMILES: c1ccc(-c2ccc3c(c2)c2cc(-c4ccccc4)ccc2n3-c2c(-c3ccccc3)cc(-c3nc(-c4ccc5c(c4)oc4ccccc45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4cccc5c4oc4ccccc45)nc(-c4cccc5c4oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4cccc5c4oc4ccccc45)n3)cc2-c2ccccc2)cc1]

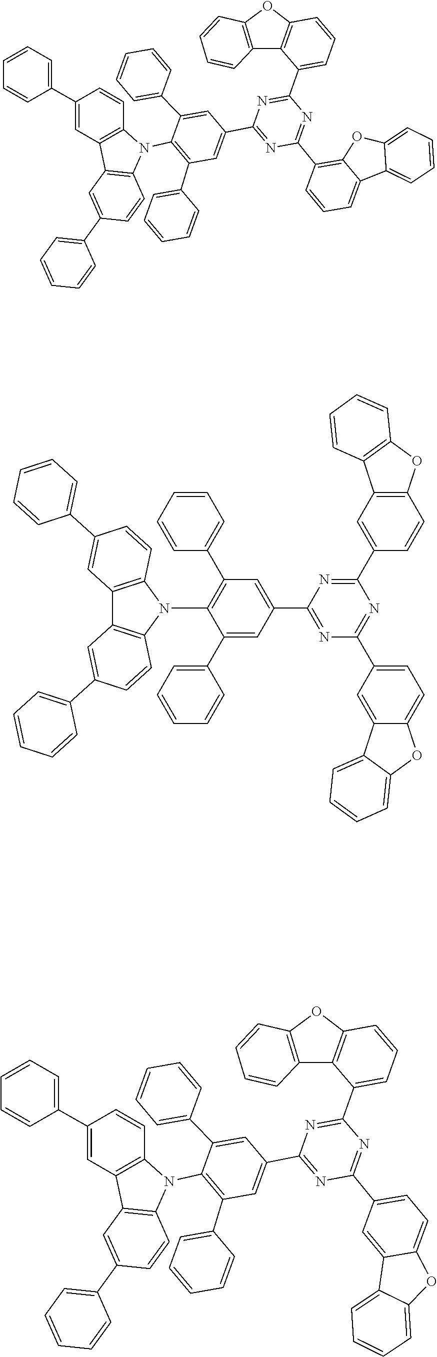 [SMILES: c1ccc(-c2ccc3c(c2)c2cc(-c4ccccc4)ccc2n3-c2c(-c3ccccc3)cc(-c3nc(-c4cccc5c4oc4ccccc45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4ccc5oc6ccccc6c5c4)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4cccc5oc6ccccc6c45)n3)cc2-c2ccccc2)cc1]

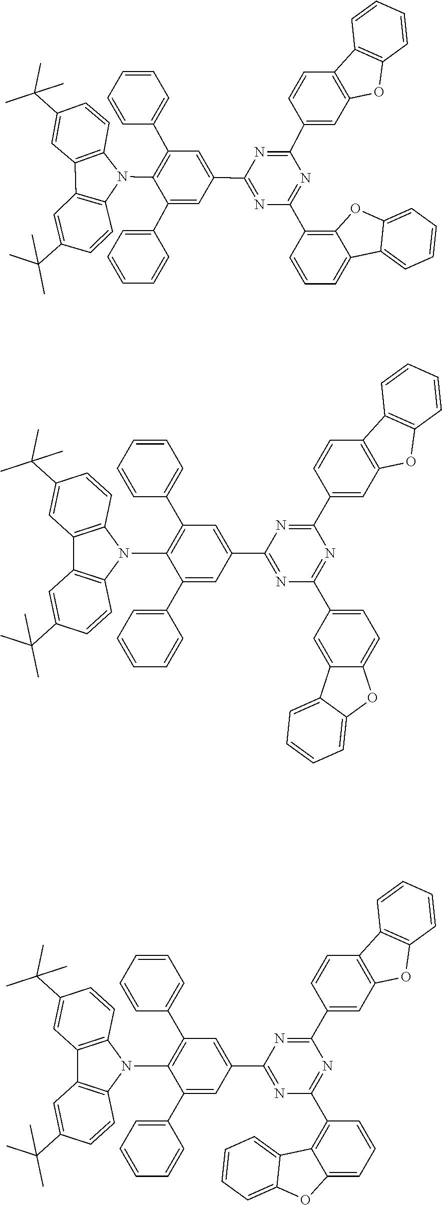 [SMILES: CC(C)(C)c1ccc2c(c1)c1cc(C(C)(C)C)ccc1n2-c1c(-c2ccccc2)cc(-c2nc(-c3ccc4c(c3)oc3ccccc34)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3cccc4oc5ccccc5c34)n2)cc1-c1ccccc1]

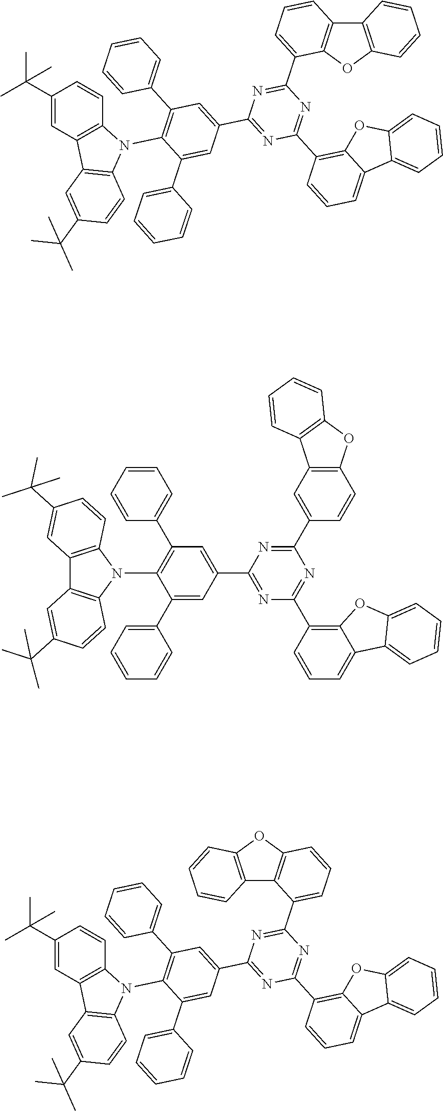 [SMILES: CC(C)(C)c1ccc2c(c1)c1cc(C(C)(C)C)ccc1n2-c1c(-c2ccccc2)cc(-c2nc(-c3cccc4c3oc3ccccc34)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc4oc5ccccc5c4c3)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3cccc4c3oc3ccccc34)nc(-c3cccc4oc5ccccc5c34)n2)cc1-c1ccccc1]

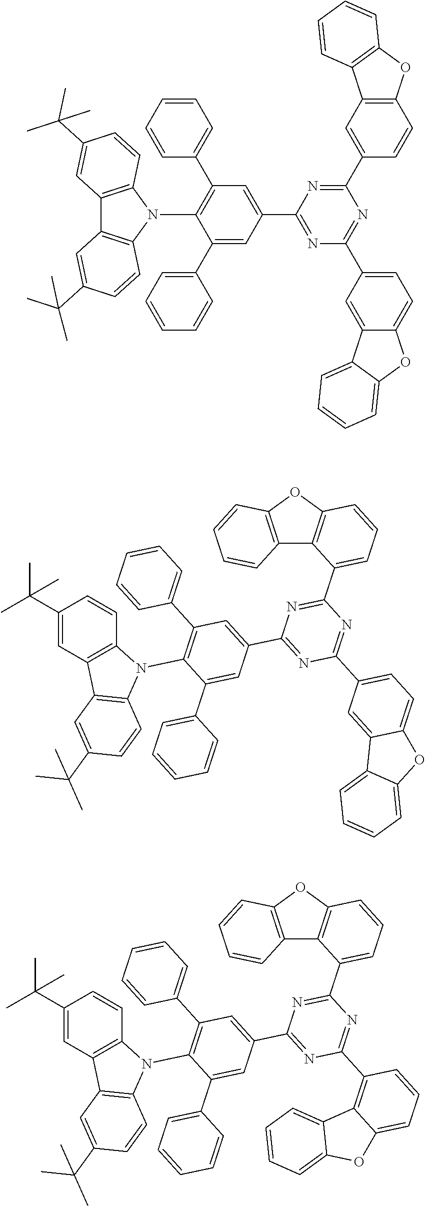 [SMILES: CC(C)(C)c1ccc2c(c1)c1cc(C(C)(C)C)ccc1n2-c1c(-c2ccccc2)cc(-c2nc(-c3ccc4oc5ccccc5c4c3)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc4oc5ccccc5c4c3)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3cccc4oc5ccccc5c34)nc(-c3cccc4oc5ccccc5c34)n2)cc1-c1ccccc1]

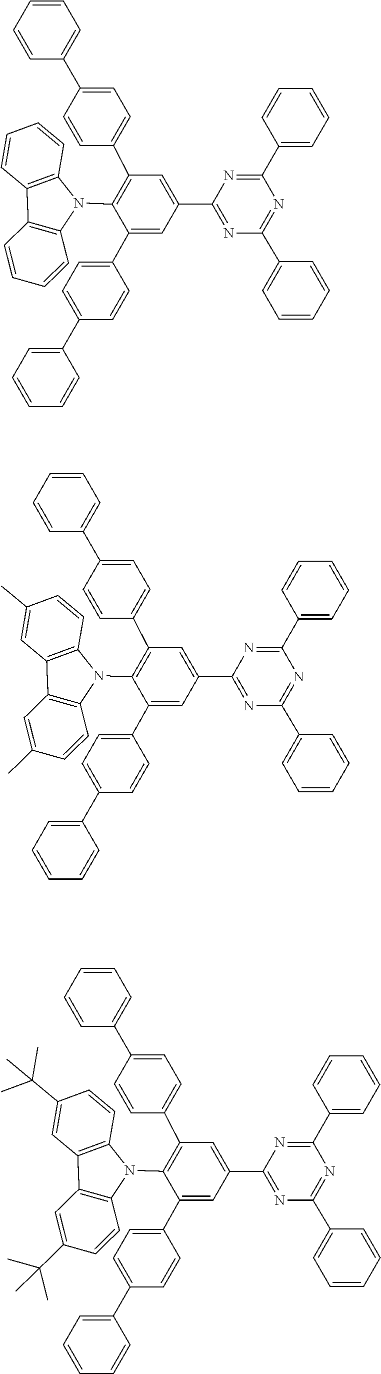 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccccc43)cc2)cc1 ; Cc1ccc2c(c1)c1cc(C)ccc1n2-c1c(-c2ccc(-c3ccccc3)cc2)cc(-c2nc(-c3ccccc3)nc(-c3ccccc3)n2)cc1-c1ccc(-c2ccccc2)cc1 ; CC(C)(C)c1ccc2c(c1)c1cc(C(C)(C)C)ccc1n2-c1c(-c2ccc(-c3ccccc3)cc2)cc(-c2nc(-c3ccccc3)nc(-c3ccccc3)n2)cc1-c1ccc(-c2ccccc2)cc1]

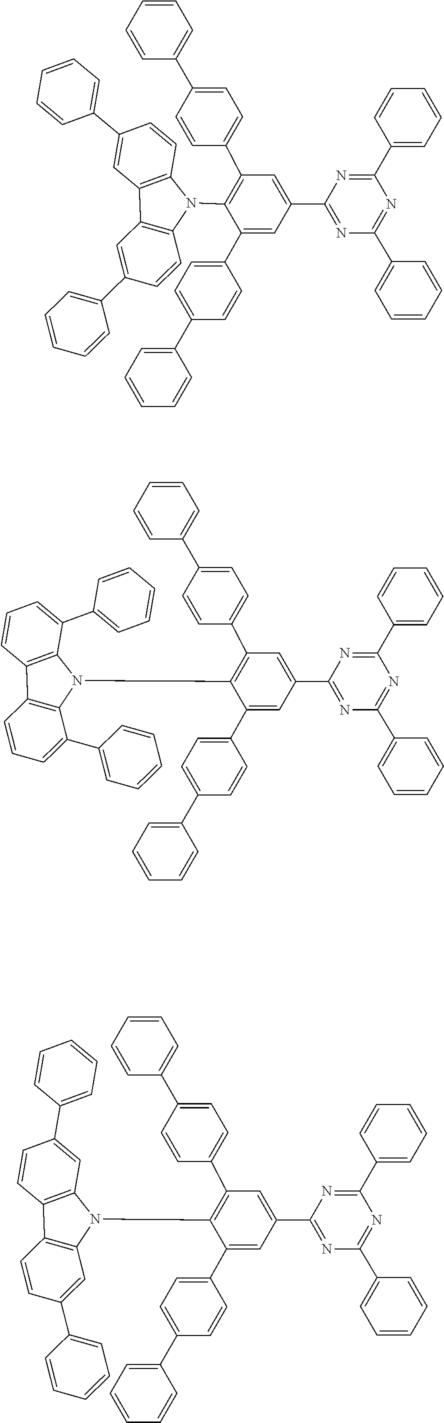 [SMILES: c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4c(-c5ccccc5)cccc4c4cccc(-c5ccccc5)c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4cc(-c5ccccc5)ccc4c4ccc(-c5ccccc5)cc43)cc2)cc1]

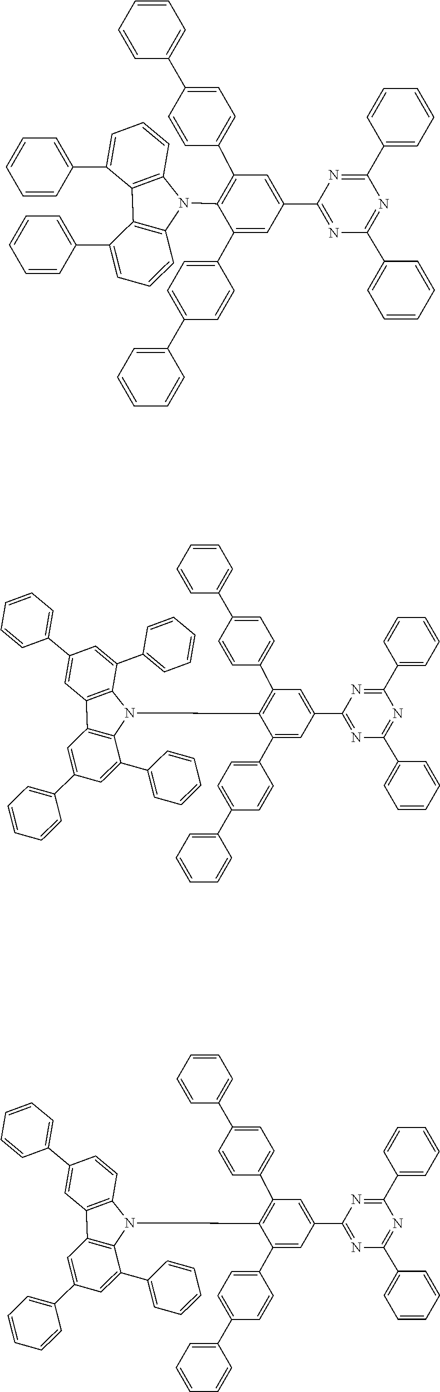 [SMILES: c1ccc(-c2ccc(-c3cc(-c4nc(-c5ccccc5)nc(-c5ccccc5)n4)cc(-c4ccc(-c5ccccc5)cc4)c3-n3c4cccc(-c5ccccc5)c4c4c(-c5ccccc5)cccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4c(-c5ccccc5)cc(-c5ccccc5)cc4c4cc(-c5ccccc5)cc(-c5ccccc5)c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4cc(-c5ccccc5)cc(-c5ccccc5)c43)cc2)cc1]

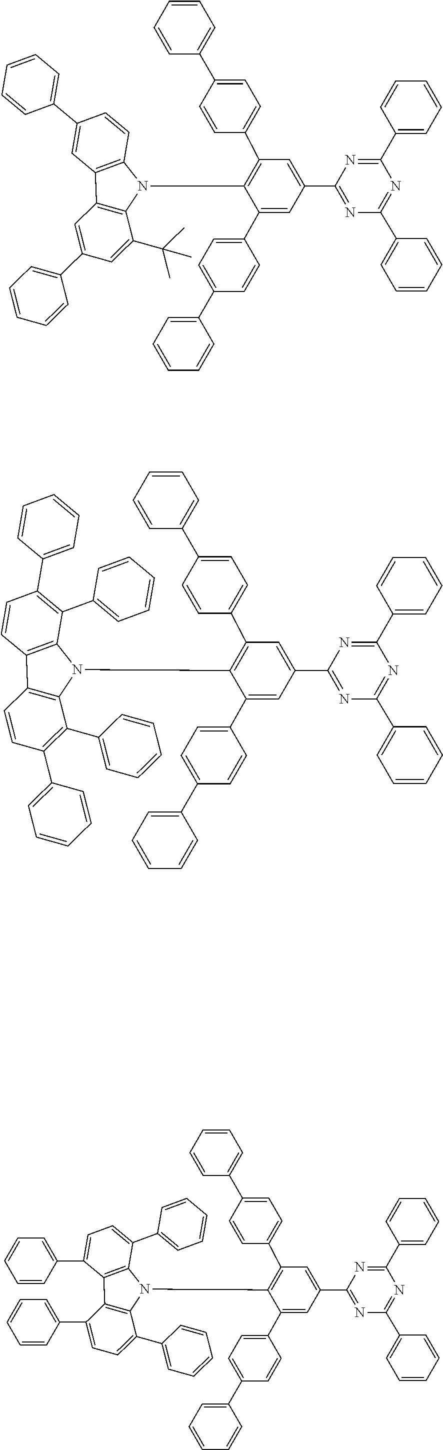 [SMILES: CC(C)(C)c1cc(-c2ccccc2)cc2c3cc(-c4ccccc4)ccc3n(-c3c(-c4ccc(-c5ccccc5)cc4)cc(-c4nc(-c5ccccc5)nc(-c5ccccc5)n4)cc3-c3ccc(-c4ccccc4)cc3)c12 ; c1ccc(-c2ccc(-c3cc(-c4nc(-c5ccccc5)nc(-c5ccccc5)n4)cc(-c4ccc(-c5ccccc5)cc4)c3-n3c4c(-c5ccccc5)c(-c5ccccc5)ccc4c4ccc(-c5ccccc5)c(-c5ccccc5)c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4c(-c5ccccc5)ccc(-c5ccccc5)c4c4c(-c5ccccc5)ccc(-c5ccccc5)c43)cc2)cc1]

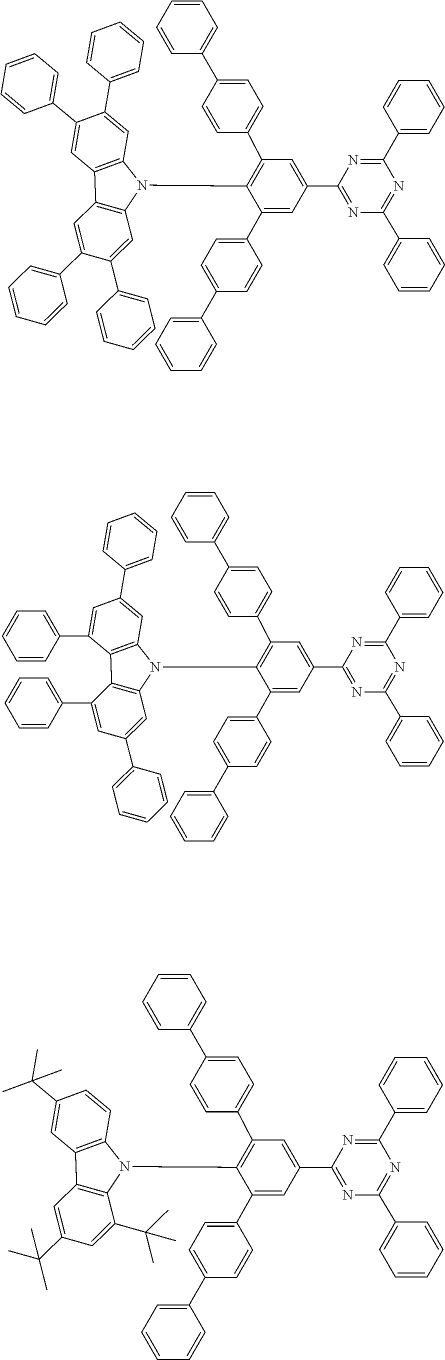 [SMILES: c1ccc(-c2ccc(-c3cc(-c4nc(-c5ccccc5)nc(-c5ccccc5)n4)cc(-c4ccc(-c5ccccc5)cc4)c3-n3c4cc(-c5ccccc5)c(-c5ccccc5)cc4c4cc(-c5ccccc5)c(-c5ccccc5)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4cc(-c5ccccc5)cc(-c5ccccc5)c4c4c(-c5ccccc5)cc(-c5ccccc5)cc43)cc2)cc1 ; CC(C)(C)c1ccc2c(c1)c1cc(C(C)(C)C)cc(C(C)(C)C)c1n2-c1c(-c2ccc(-c3ccccc3)cc2)cc(-c2nc(-c3ccccc3)nc(-c3ccccc3)n2)cc1-c1ccc(-c2ccccc2)cc1]

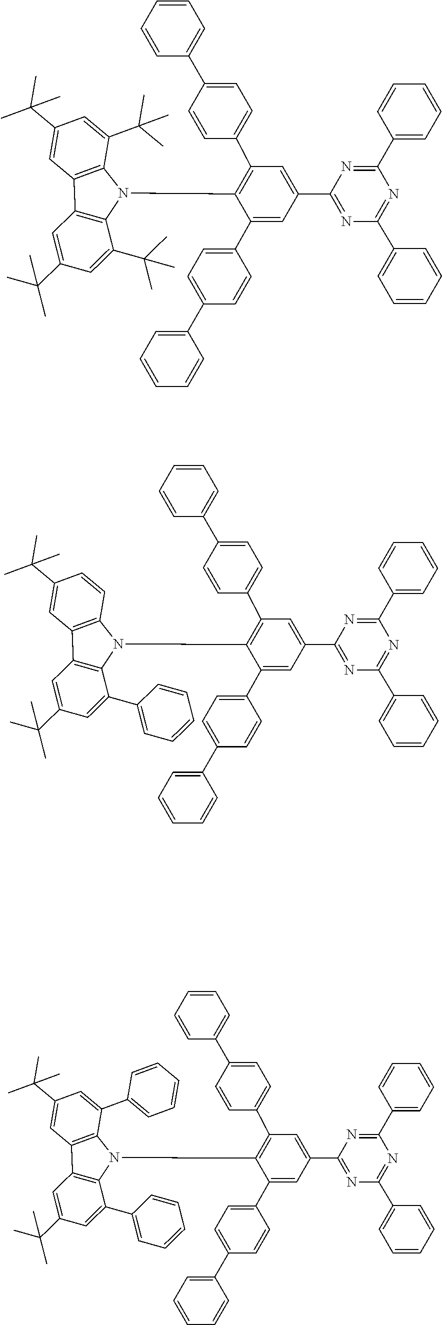 [SMILES: CC(C)(C)c1cc(C(C)(C)C)c2c(c1)c1cc(C(C)(C)C)cc(C(C)(C)C)c1n2-c1c(-c2ccc(-c3ccccc3)cc2)cc(-c2nc(-c3ccccc3)nc(-c3ccccc3)n2)cc1-c1ccc(-c2ccccc2)cc1 ; CC(C)(C)c1ccc2c(c1)c1cc(C(C)(C)C)cc(-c3ccccc3)c1n2-c1c(-c2ccc(-c3ccccc3)cc2)cc(-c2nc(-c3ccccc3)nc(-c3ccccc3)n2)cc1-c1ccc(-c2ccccc2)cc1 ; CC(C)(C)c1cc(-c2ccccc2)c2c(c1)c1cc(C(C)(C)C)cc(-c3ccccc3)c1n2-c1c(-c2ccc(-c3ccccc3)cc2)cc(-c2nc(-c3ccccc3)nc(-c3ccccc3)n2)cc1-c1ccc(-c2ccccc2)cc1]

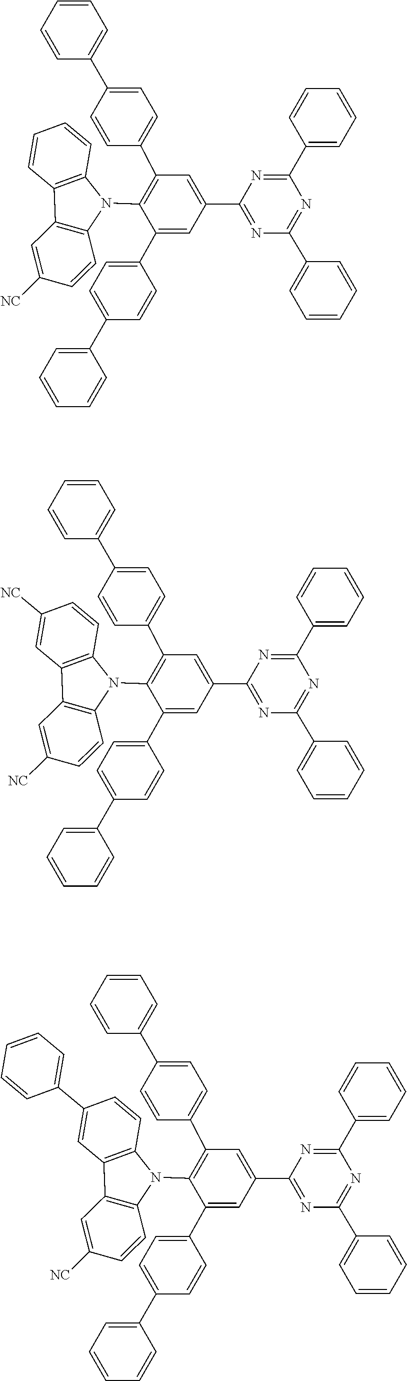 [SMILES: N#Cc1ccc2c(c1)c1ccccc1n2-c1c(-c2ccc(-c3ccccc3)cc2)cc(-c2nc(-c3ccccc3)nc(-c3ccccc3)n2)cc1-c1ccc(-c2ccccc2)cc1 ; N#Cc1ccc2c(c1)c1cc(C#N)ccc1n2-c1c(-c2ccc(-c3ccccc3)cc2)cc(-c2nc(-c3ccccc3)nc(-c3ccccc3)n2)cc1-c1ccc(-c2ccccc2)cc1 ; N#Cc1ccc2c(c1)c1cc(-c3ccccc3)ccc1n2-c1c(-c2ccc(-c3ccccc3)cc2)cc(-c2nc(-c3ccccc3)nc(-c3ccccc3)n2)cc1-c1ccc(-c2ccccc2)cc1]

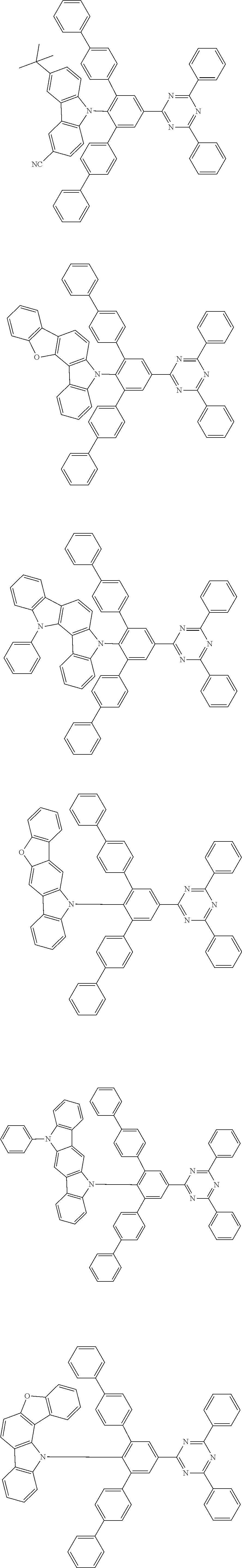

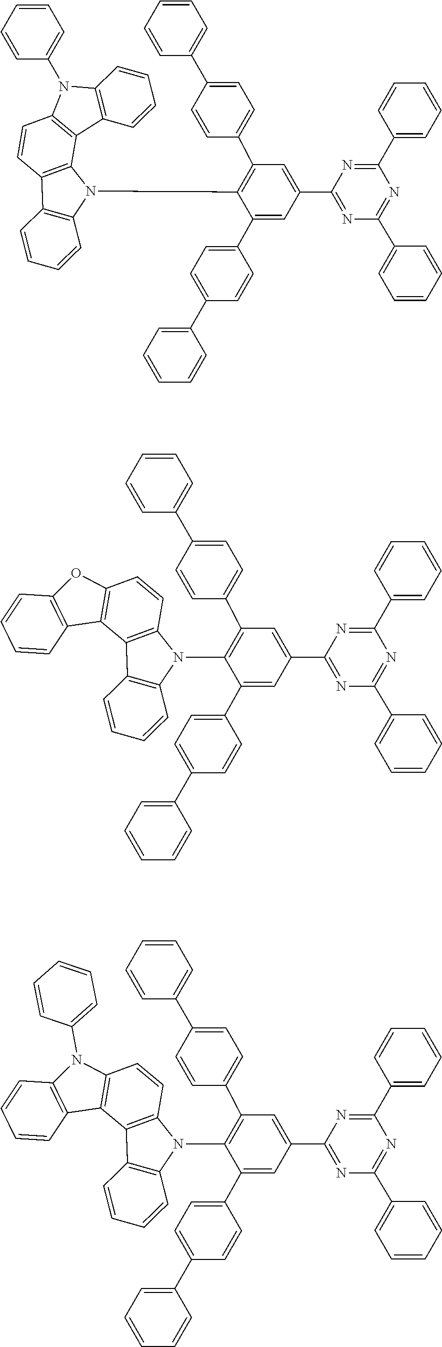 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c(c6ccccc6n5-c5ccccc5)c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5c(ccc43)oc3ccccc35)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5c6ccccc6n(-c6ccccc6)c5ccc43)cc2)cc1]

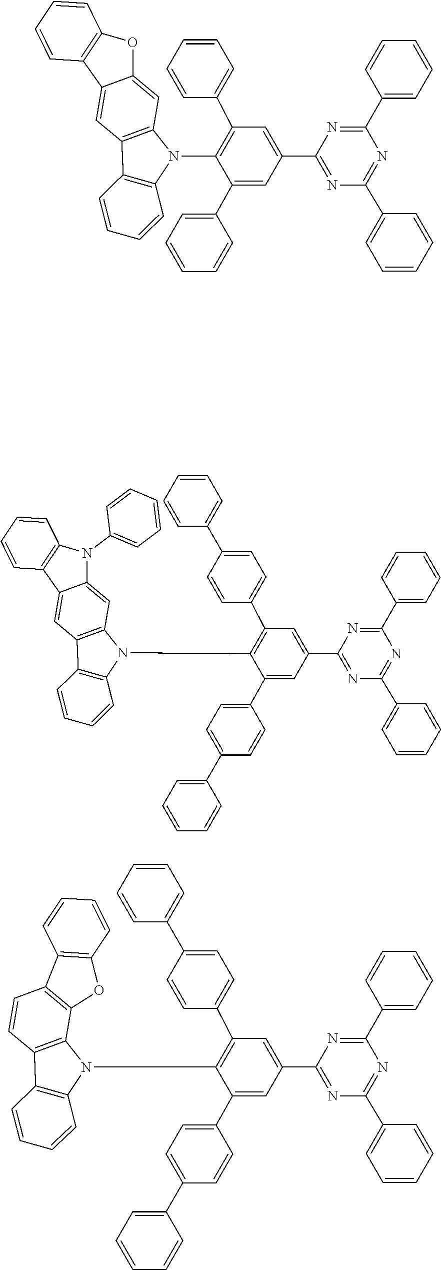

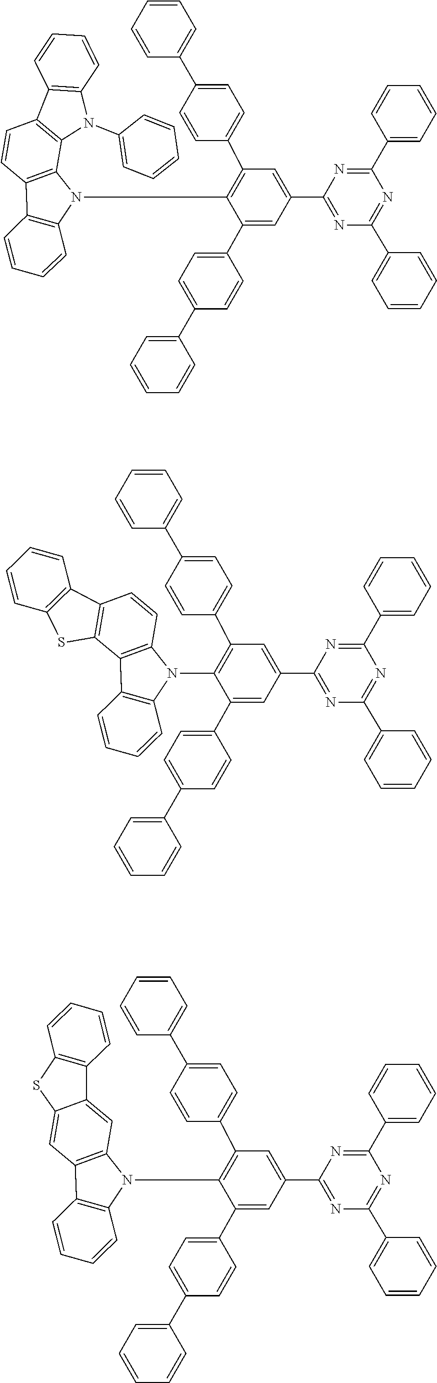 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c6ccccc6n(-c6ccccc6)c5c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6ccccc6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc5sc6ccccc6c5cc43)cc2)cc1]

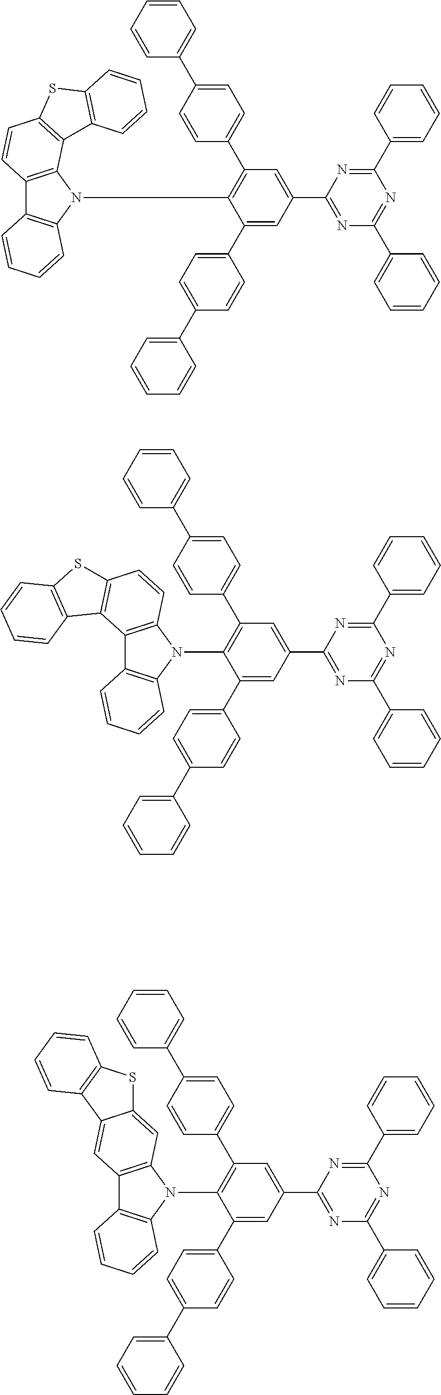 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5sc6ccccc6c5c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5c(ccc43)sc3ccccc35)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc5c(cc43)sc3ccccc35)cc2)cc1]

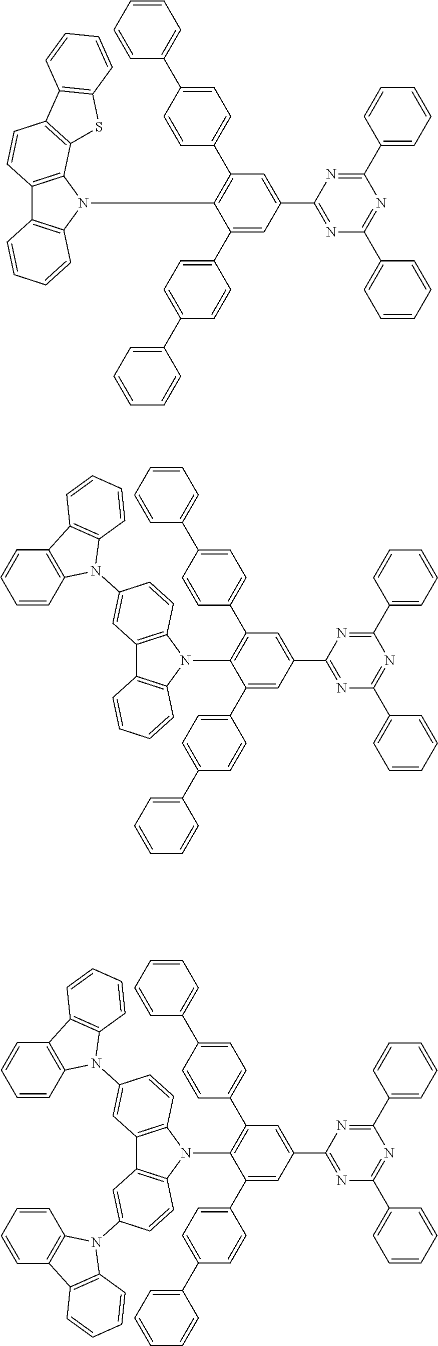 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c6ccccc6sc5c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc(-n5c6ccccc6c6ccccc65)ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4cc(-n5c6ccccc6c6ccccc65)ccc43)cc2)cc1]

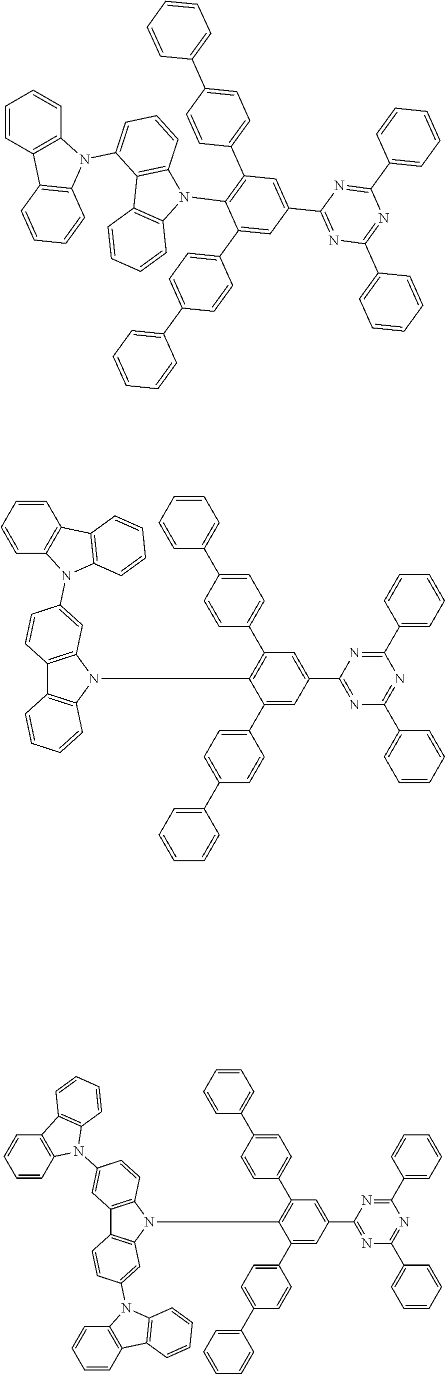 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c(-n5c6ccccc6c6ccccc65)cccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc(-n5c6ccccc6c6ccccc65)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4ccc(-n5c6ccccc6c6ccccc65)cc43)cc2)cc1]

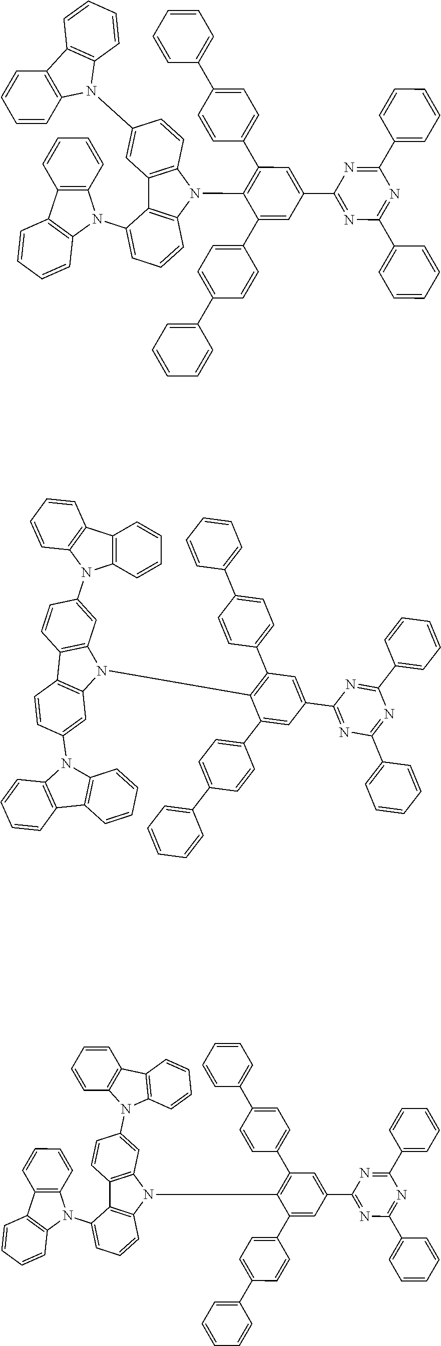 [SMILES: c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4c(-n5c6ccccc6c6ccccc65)cccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)ccc4c4ccc(-n5c6ccccc6c6ccccc65)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)ccc4c4c(-n5c6ccccc6c6ccccc65)cccc43)cc2)cc1]

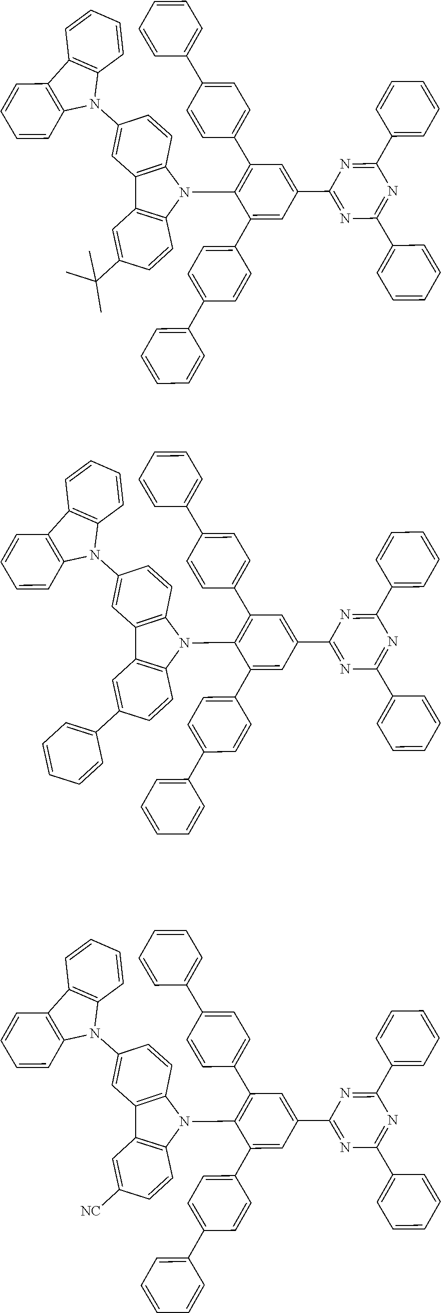 [SMILES: CC(C)(C)c1ccc2c(c1)c1cc(-n3c4ccccc4c4ccccc43)ccc1n2-c1c(-c2ccc(-c3ccccc3)cc2)cc(-c2nc(-c3ccccc3)nc(-c3ccccc3)n2)cc1-c1ccc(-c2ccccc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4cc(-n5c6ccccc6c6ccccc65)ccc43)cc2)cc1 ; N#Cc1ccc2c(c1)c1cc(-n3c4ccccc4c4ccccc43)ccc1n2-c1c(-c2ccc(-c3ccccc3)cc2)cc(-c2nc(-c3ccccc3)nc(-c3ccccc3)n2)cc1-c1ccc(-c2ccccc2)cc1]

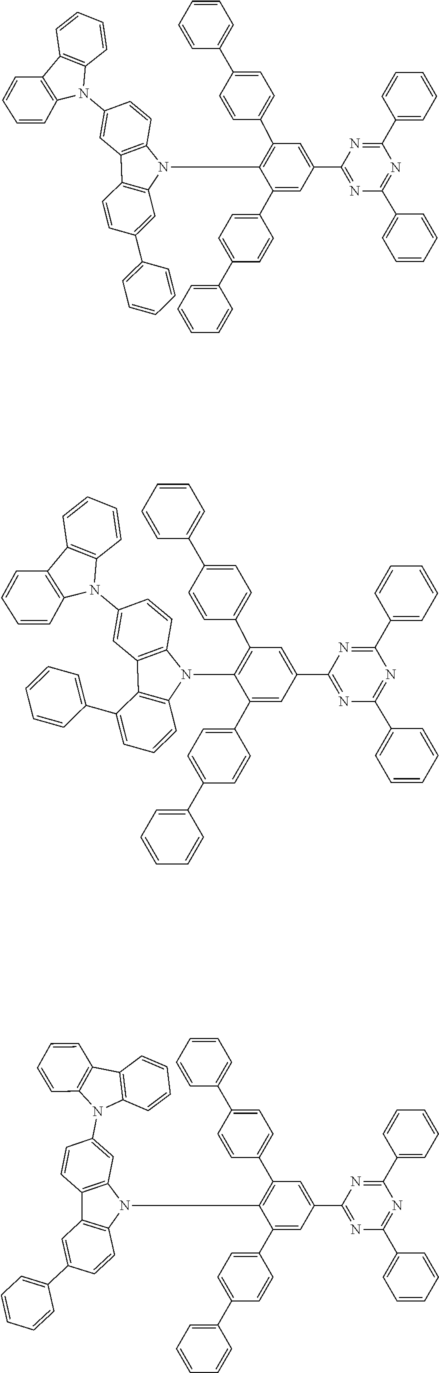 [SMILES: c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4ccc(-c5ccccc5)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4c(-c5ccccc5)cccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4ccc(-n5c6ccccc6c6ccccc65)cc43)cc2)cc1]

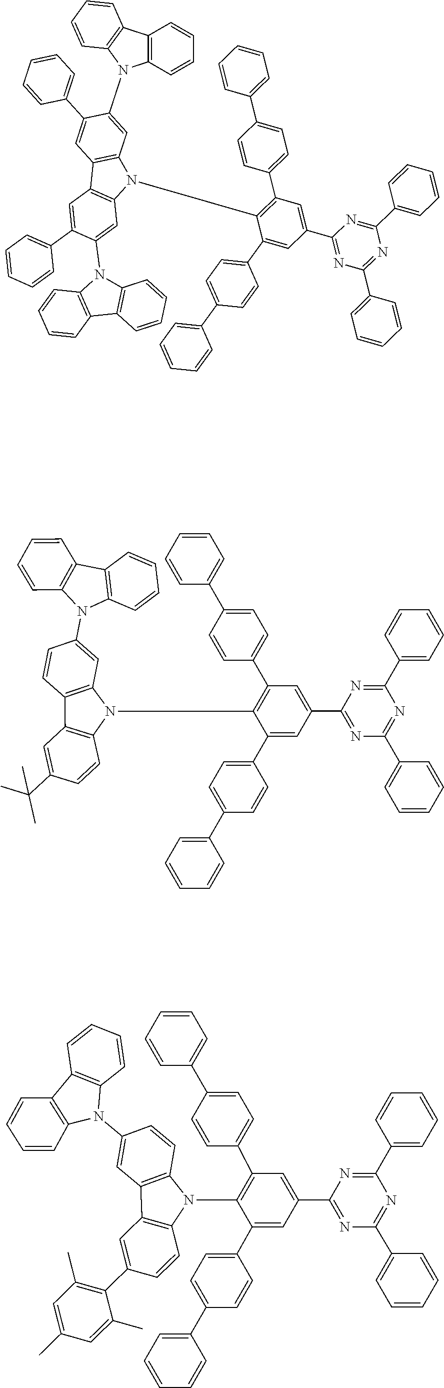 [SMILES: c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)c(-c5ccccc5)cc4c4cc(-c5ccccc5)c(-n5c6ccccc6c6ccccc65)cc43)cc2)cc1 ; CC(C)(C)c1ccc2c(c1)c1ccc(-n3c4ccccc4c4ccccc43)cc1n2-c1c(-c2ccc(-c3ccccc3)cc2)cc(-c2nc(-c3ccccc3)nc(-c3ccccc3)n2)cc1-c1ccc(-c2ccccc2)cc1 ; Cc1cc(C)c(-c2ccc3c(c2)c2cc(-n4c5ccccc5c5ccccc54)ccc2n3-c2c(-c3ccc(-c4ccccc4)cc3)cc(-c3nc(-c4ccccc4)nc(-c4ccccc4)n3)cc2-c2ccc(-c3ccccc3)cc2)c(C)c1]

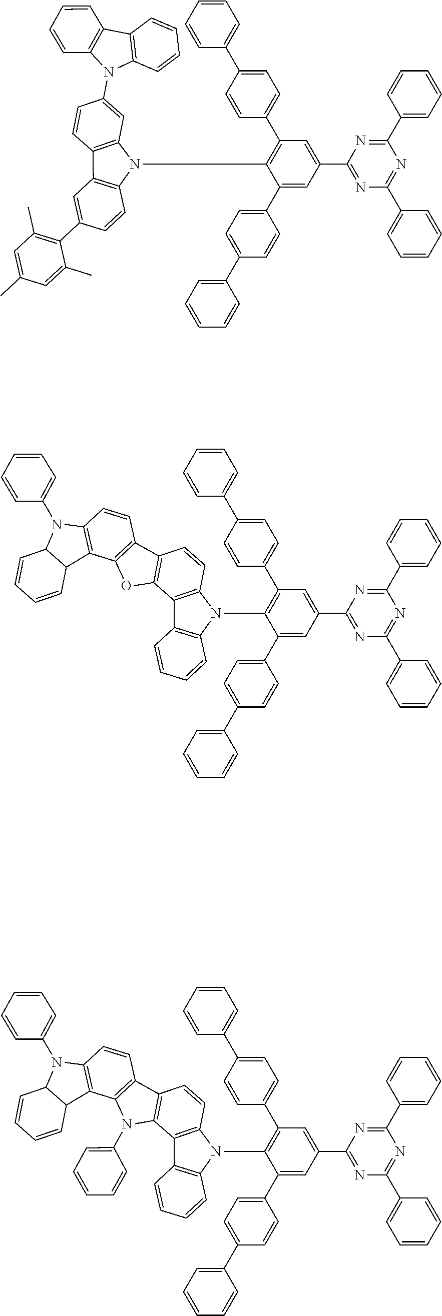 [SMILES: Cc1cc(C)c(-c2ccc3c(c2)c2ccc(-n4c5ccccc5c5ccccc54)cc2n3-c2c(-c3ccc(-c4ccccc4)cc3)cc(-c3nc(-c4ccccc4)nc(-c4ccccc4)n3)cc2-c2ccc(-c3ccccc3)cc2)c(C)c1 ; C1=CC2c3c(ccc4c3oc3c4ccc4c3c3ccccc3n4-c3c(-c4ccc(-c5ccccc5)cc4)cc(-c4nc(-c5ccccc5)nc(-c5ccccc5)n4)cc3-c3ccc(-c4ccccc4)cc3)N(c3ccccc3)C2C=C1 ; C1=CC2c3c(ccc4c5ccc6c(c7ccccc7n6-c6c(-c7ccc(-c8ccccc8)cc7)cc(-c7nc(-c8ccccc8)nc(-c8ccccc8)n7)cc6-c6ccc(-c7ccccc7)cc6)c5n(-c5ccccc5)c34)N(c3ccccc3)C2C=C1]

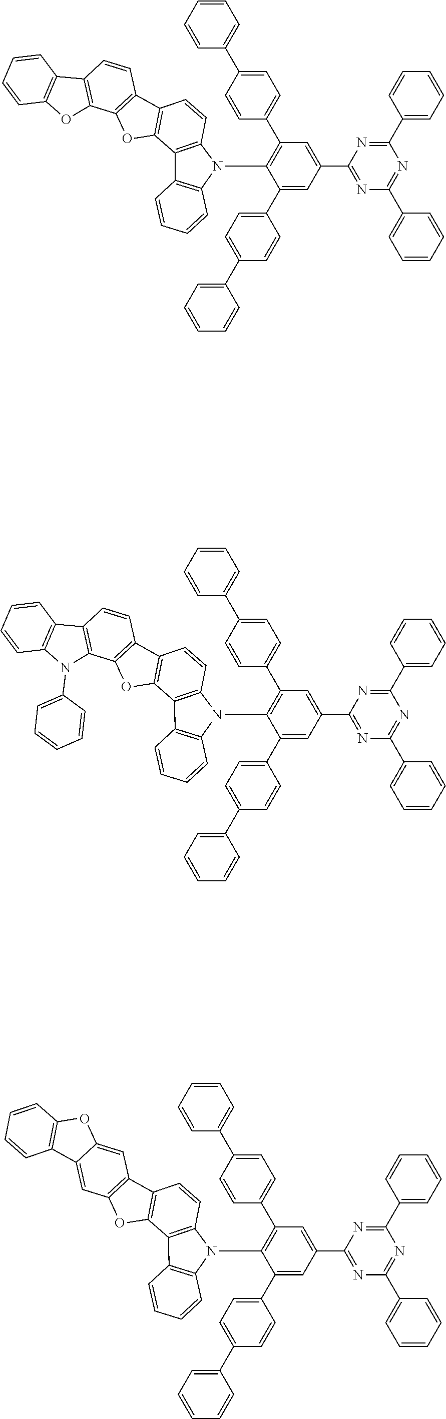 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7c8ccccc8oc76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7c8ccccc8n(-c8ccccc8)c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c(cc6c5ccc43)oc3ccccc37)cc2)cc1]

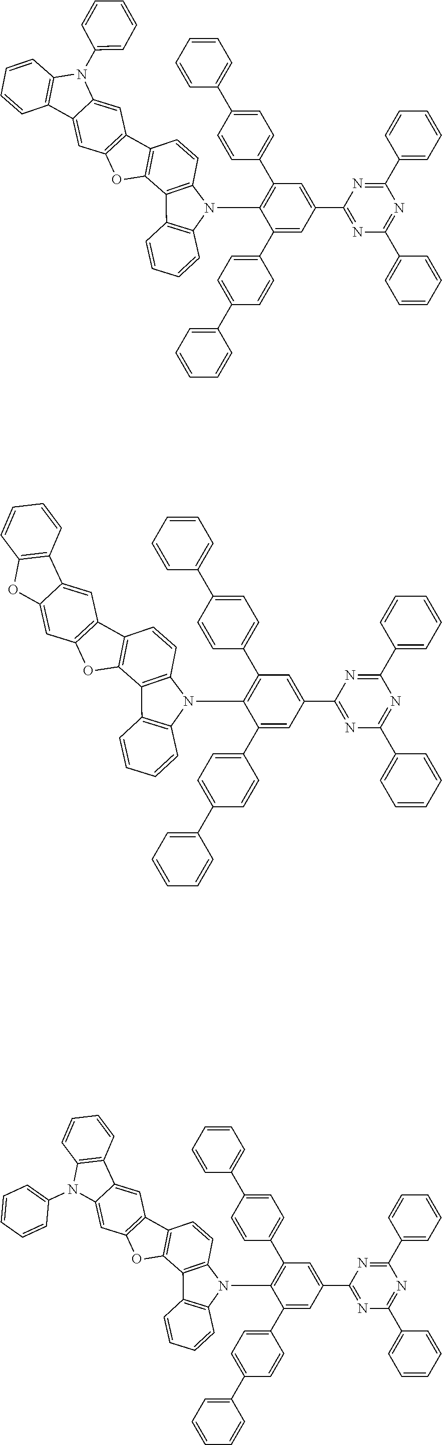 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c8ccccc8n(-c8ccccc8)c7cc6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7oc8ccccc8c7cc6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c(cc6c5ccc43)c3ccccc3n7-c3ccccc3)cc2)cc1]

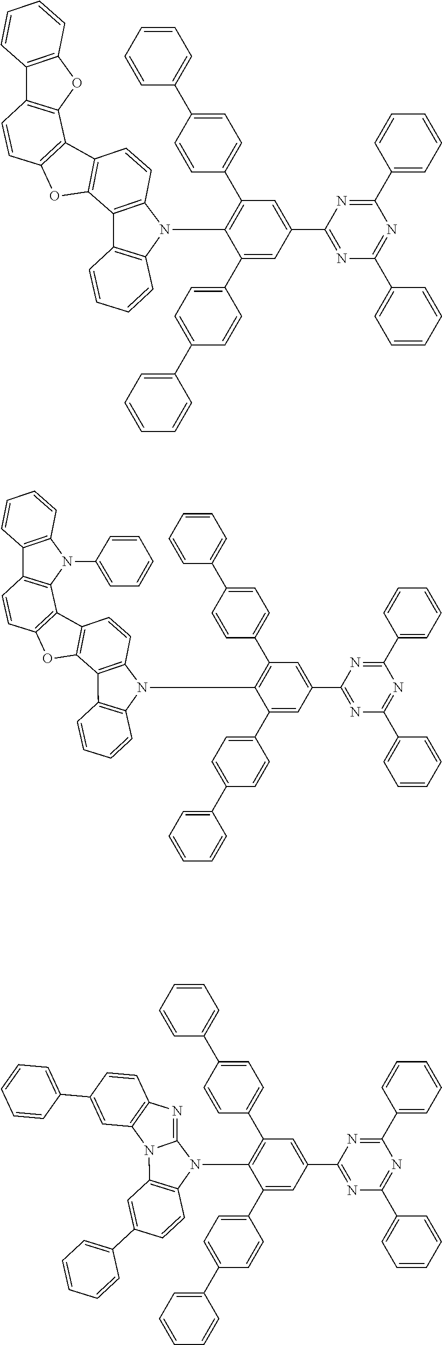 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6ccc7c8ccccc8oc7c6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6ccc7c8ccccc8n(-c8ccccc8)c7c6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4n4c5cc(-c6ccccc6)ccc5nc34)cc2)cc1]

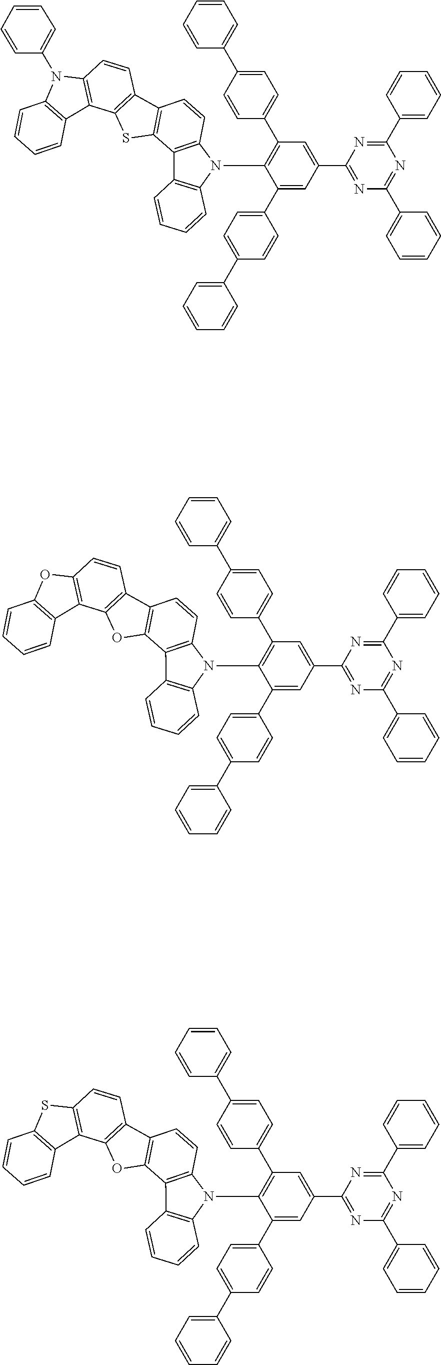 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7c6c6ccccc6n7-c6ccccc6)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7oc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7sc8ccccc8c76)c5ccc43)cc2)cc1]

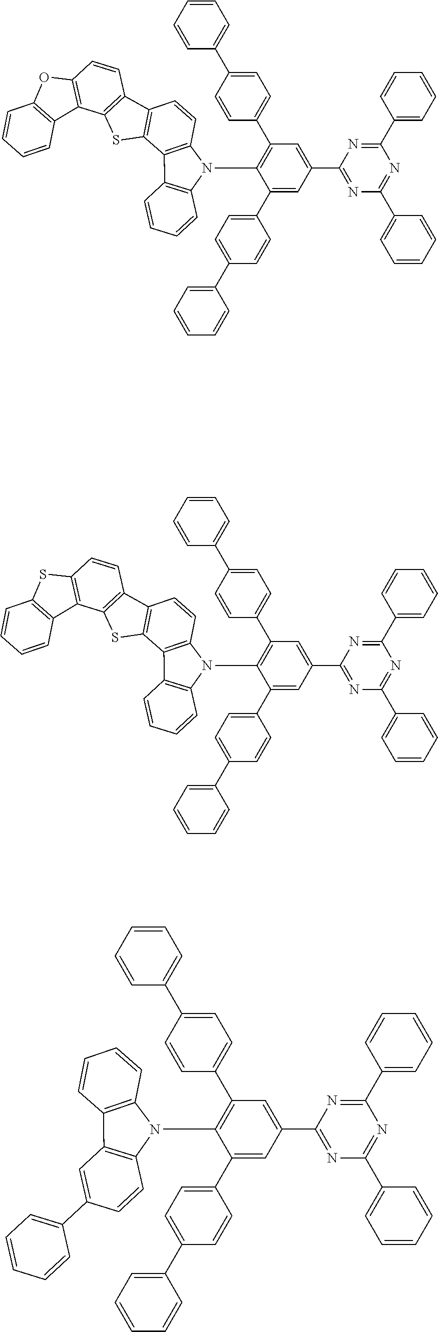 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7oc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7sc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc(-c5ccccc5)ccc43)cc2)cc1]

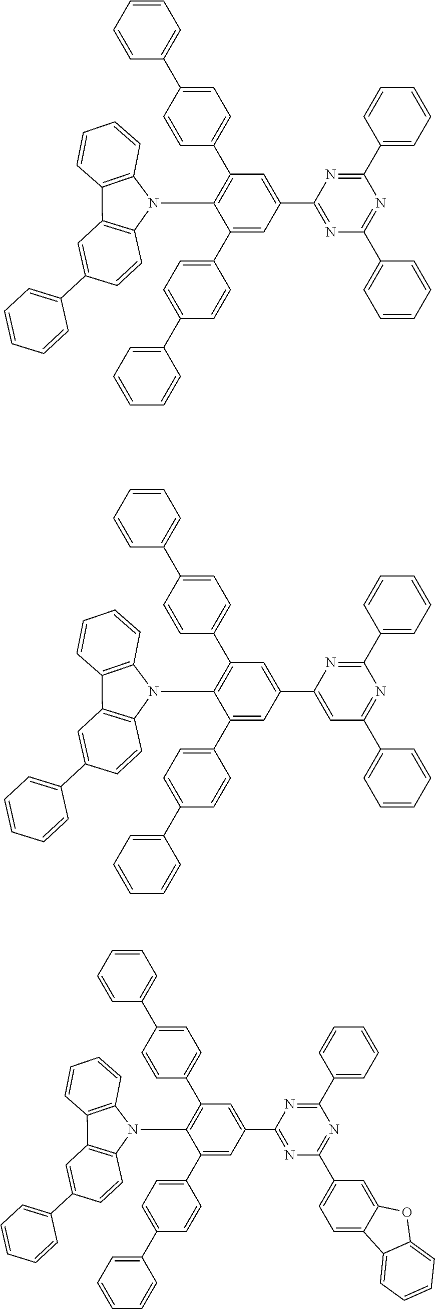 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4cc(-c5ccccc5)ccc43)cc2)cc1 ; c1ccc(-c2ccc(-c3cc(-c4cc(-c5ccccc5)nc(-c5ccccc5)n4)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc(-c5ccc6c(c5)oc5ccccc56)n4)cc(-c4ccc(-c5ccccc5)cc4)c3-n3c4ccccc4c4cc(-c5ccccc5)ccc43)cc2)cc1]

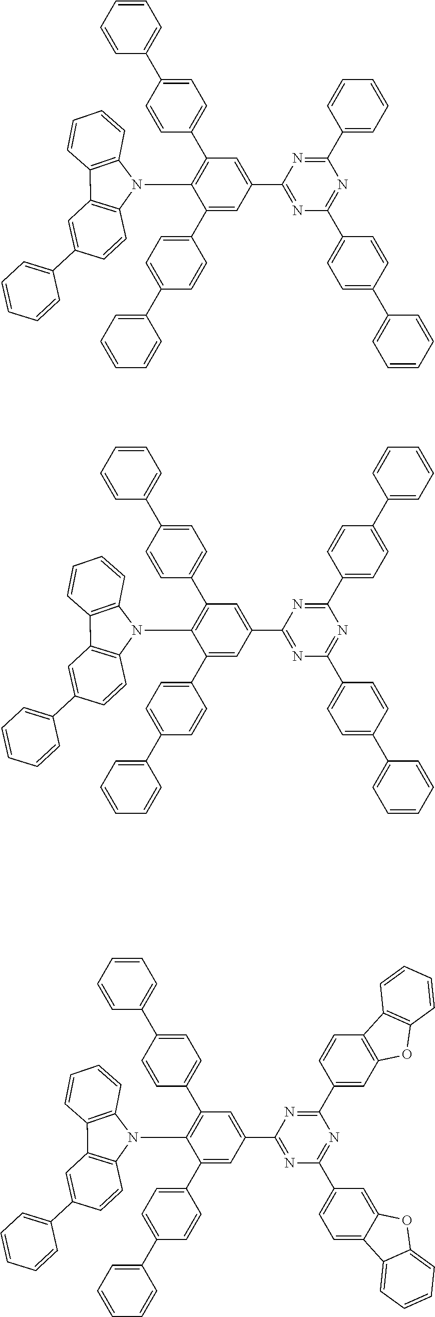 [SMILES: c1ccc(-c2ccc(-c3nc(-c4ccccc4)nc(-c4cc(-c5ccc(-c6ccccc6)cc5)c(-n5c6ccccc6c6cc(-c7ccccc7)ccc65)c(-c5ccc(-c6ccccc6)cc5)c4)n3)cc2)cc1 ; c1ccc(-c2ccc(-c3nc(-c4ccc(-c5ccccc5)cc4)nc(-c4cc(-c5ccc(-c6ccccc6)cc5)c(-n5c6ccccc6c6cc(-c7ccccc7)ccc65)c(-c5ccc(-c6ccccc6)cc5)c4)n3)cc2)cc1 ; c1ccc(-c2ccc(-c3cc(-c4nc(-c5ccc6c(c5)oc5ccccc56)nc(-c5ccc6c(c5)oc5ccccc56)n4)cc(-c4ccc(-c5ccccc5)cc4)c3-n3c4ccccc4c4cc(-c5ccccc5)ccc43)cc2)cc1]

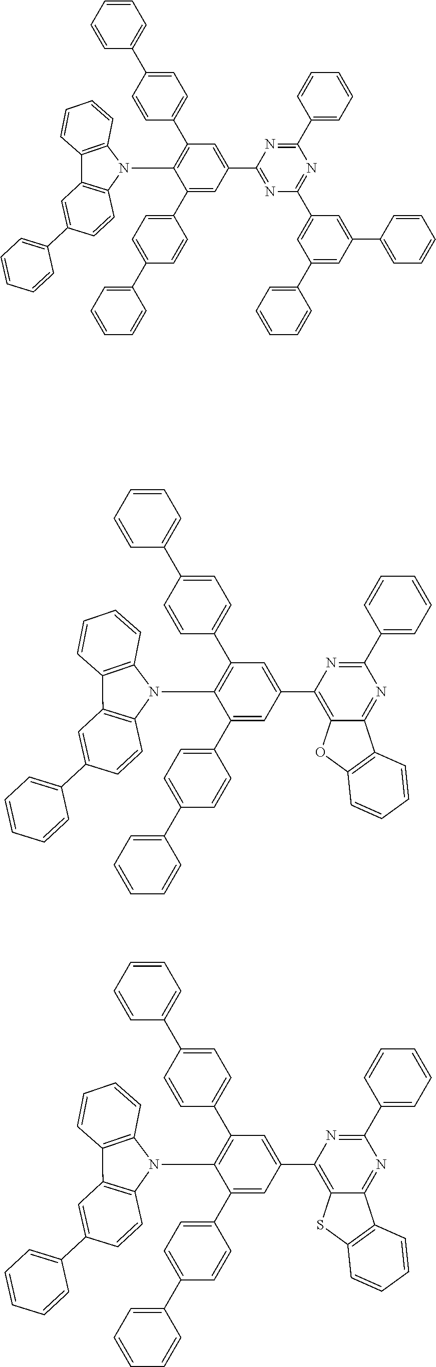 [SMILES: c1ccc(-c2ccc(-c3cc(-c4nc(-c5ccccc5)nc(-c5cc(-c6ccccc6)cc(-c6ccccc6)c5)n4)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc5c4oc4ccccc45)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc5c4sc4ccccc45)cc(-c4ccc(-c5ccccc5)cc4)c3-n3c4ccccc4c4cc(-c5ccccc5)ccc43)cc2)cc1]

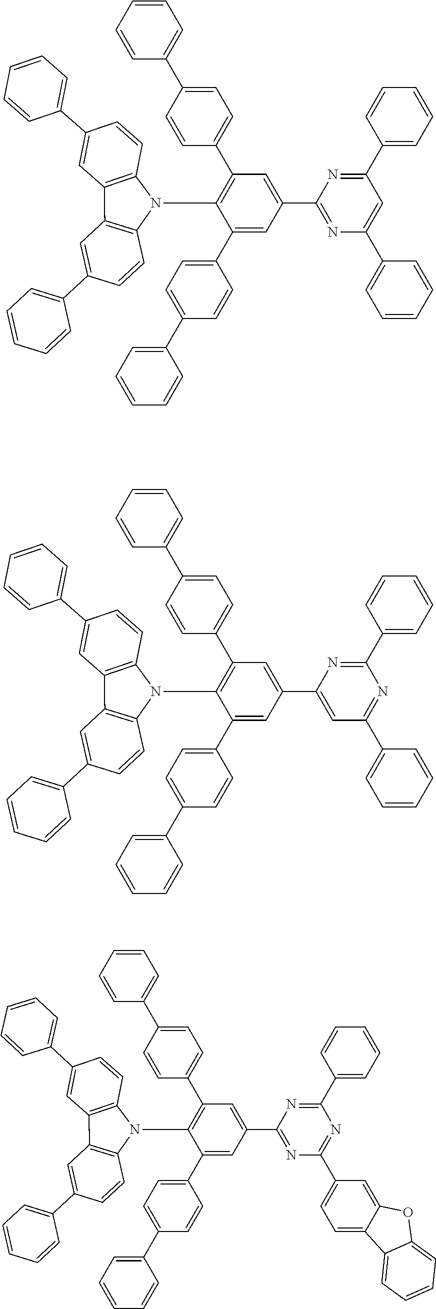 [SMILES: c1ccc(-c2ccc(-c3cc(-c4nc(-c5ccccc5)cc(-c5ccccc5)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4cc(-c5ccccc5)nc(-c5ccccc5)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc(-c5ccc6c(c5)oc5ccccc56)n4)cc(-c4ccc(-c5ccccc5)cc4)c3-n3c4ccc(-c5ccccc5)cc4c4cc(-c5ccccc5)ccc43)cc2)cc1]

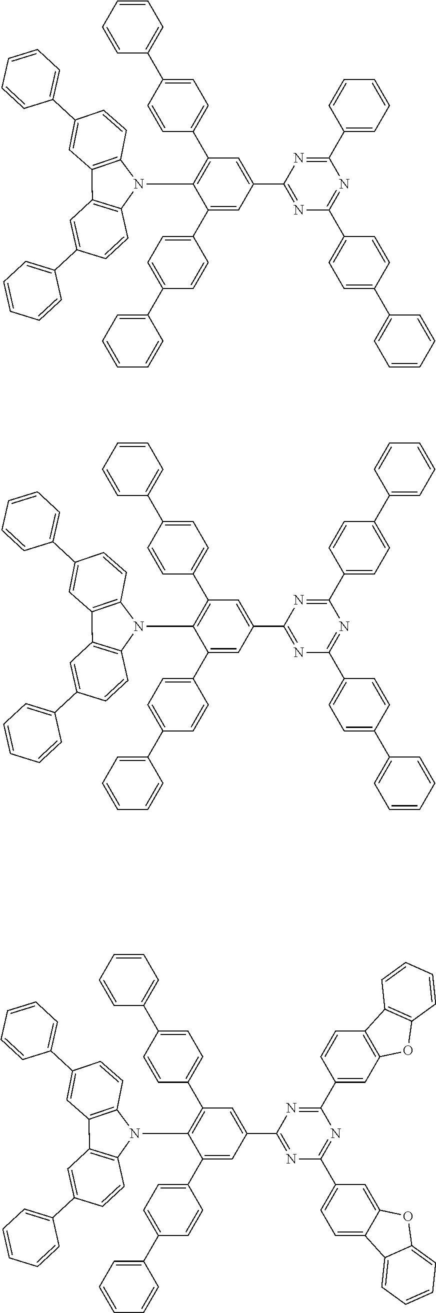 [SMILES: c1ccc(-c2ccc(-c3nc(-c4ccccc4)nc(-c4cc(-c5ccc(-c6ccccc6)cc5)c(-n5c6ccc(-c7ccccc7)cc6c6cc(-c7ccccc7)ccc65)c(-c5ccc(-c6ccccc6)cc5)c4)n3)cc2)cc1 ; c1ccc(-c2ccc(-c3nc(-c4ccc(-c5ccccc5)cc4)nc(-c4cc(-c5ccc(-c6ccccc6)cc5)c(-n5c6ccc(-c7ccccc7)cc6c6cc(-c7ccccc7)ccc65)c(-c5ccc(-c6ccccc6)cc5)c4)n3)cc2)cc1 ; c1ccc(-c2ccc(-c3cc(-c4nc(-c5ccc6c(c5)oc5ccccc56)nc(-c5ccc6c(c5)oc5ccccc56)n4)cc(-c4ccc(-c5ccccc5)cc4)c3-n3c4ccc(-c5ccccc5)cc4c4cc(-c5ccccc5)ccc43)cc2)cc1]

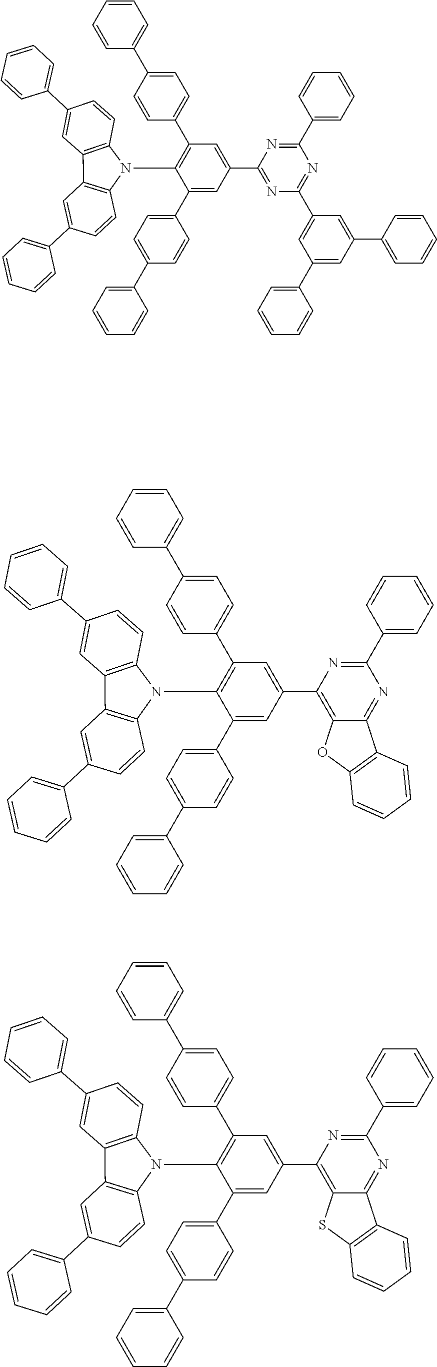 [SMILES: c1ccc(-c2ccc(-c3cc(-c4nc(-c5ccccc5)nc(-c5cc(-c6ccccc6)cc(-c6ccccc6)c5)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc5c4oc4ccccc45)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc5c4sc4ccccc45)cc(-c4ccc(-c5ccccc5)cc4)c3-n3c4ccc(-c5ccccc5)cc4c4cc(-c5ccccc5)ccc43)cc2)cc1]

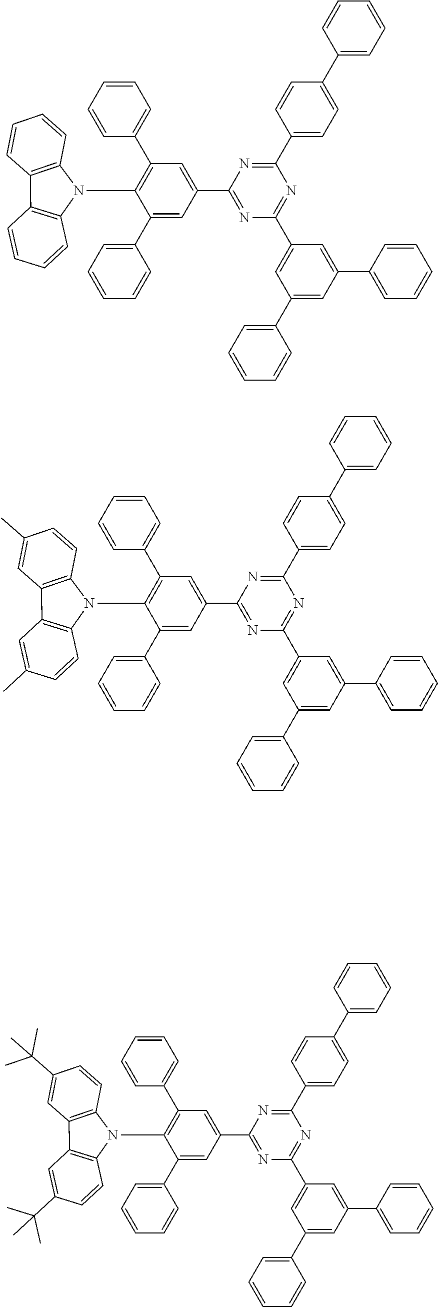 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccccc65)c(-c5ccccc5)c4)n3)cc2)cc1 ; Cc1ccc2c(c1)c1cc(C)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

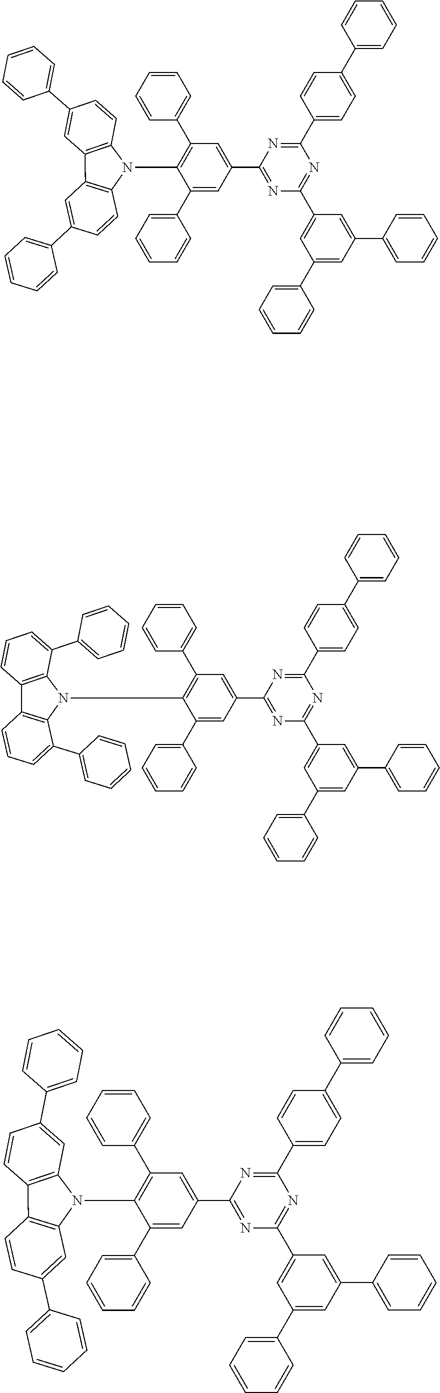 [SMILES: c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)cccc6c6cccc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)ccc6c6ccc(-c7ccccc7)cc65)c(-c5ccccc5)c4)n3)cc2)cc1]

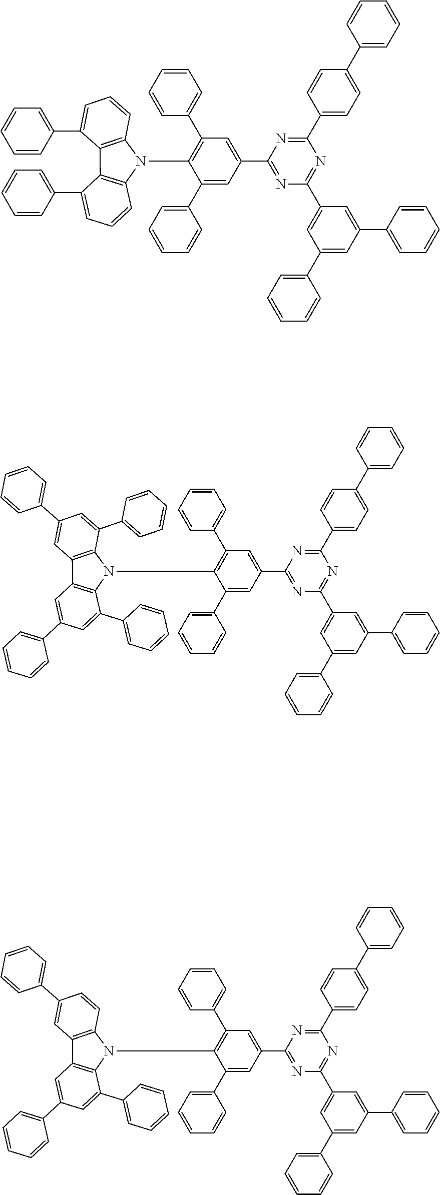 [SMILES: c1ccc(-c2ccc(-c3nc(-c4cc(-c5ccccc5)cc(-c5ccccc5)c4)nc(-c4cc(-c5ccccc5)c(-n5c6cccc(-c7ccccc7)c6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)cc(-c7ccccc7)cc6c6cc(-c7ccccc7)cc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)cc(-c7ccccc7)c65)c(-c5ccccc5)c4)n3)cc2)cc1]

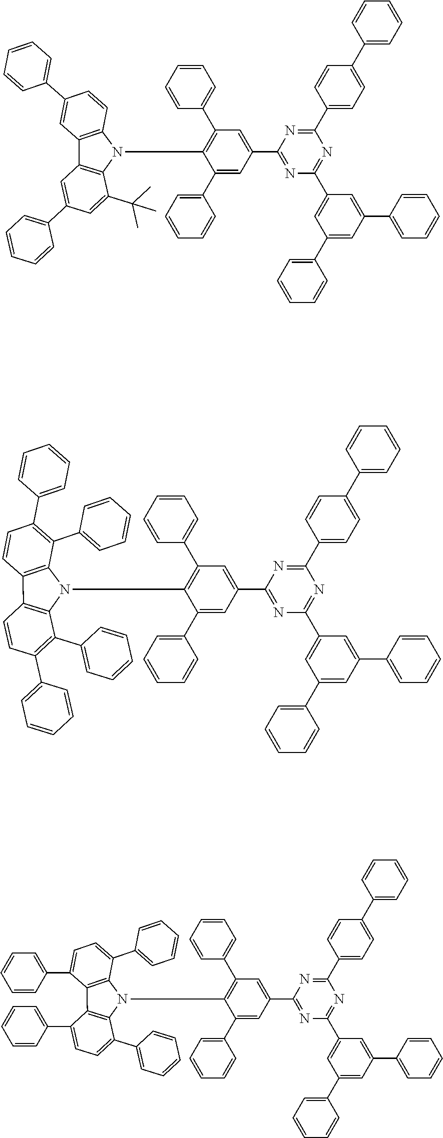 [SMILES: CC(C)(C)c1cc(-c2ccccc2)cc2c3cc(-c4ccccc4)ccc3n(-c3c(-c4ccccc4)cc(-c4nc(-c5ccc(-c6ccccc6)cc5)nc(-c5cc(-c6ccccc6)cc(-c6ccccc6)c5)n4)cc3-c3ccccc3)c12 ; c1ccc(-c2ccc(-c3nc(-c4cc(-c5ccccc5)cc(-c5ccccc5)c4)nc(-c4cc(-c5ccccc5)c(-n5c6c(-c7ccccc7)c(-c7ccccc7)ccc6c6ccc(-c7ccccc7)c(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)ccc(-c7ccccc7)c6c6c(-c7ccccc7)ccc(-c7ccccc7)c65)c(-c5ccccc5)c4)n3)cc2)cc1]

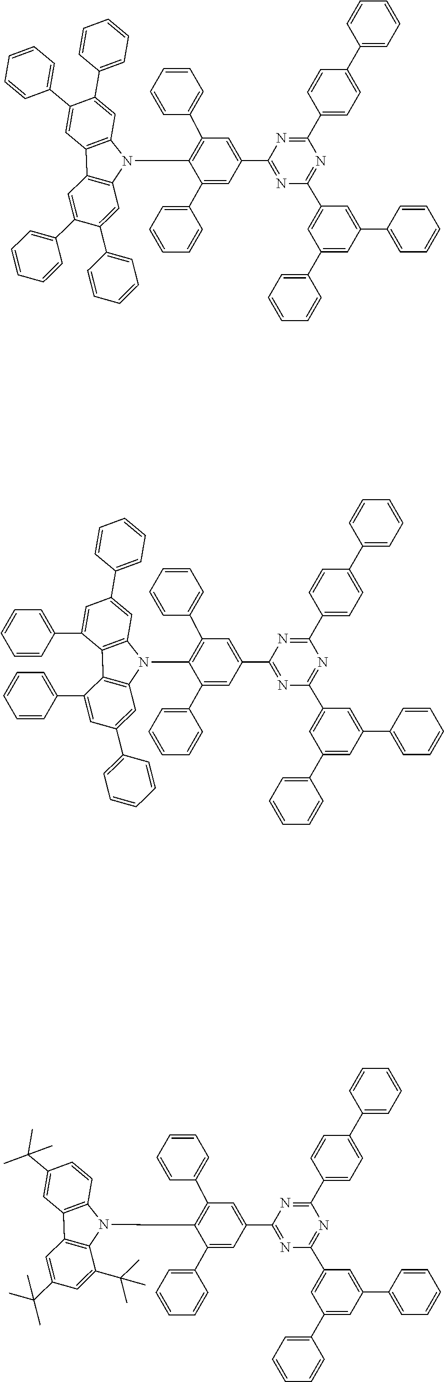 [SMILES: c1ccc(-c2ccc(-c3nc(-c4cc(-c5ccccc5)cc(-c5ccccc5)c4)nc(-c4cc(-c5ccccc5)c(-n5c6cc(-c7ccccc7)c(-c7ccccc7)cc6c6cc(-c7ccccc7)c(-c7ccccc7)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)cc(-c7ccccc7)c6c6c(-c7ccccc7)cc(-c7ccccc7)cc65)c(-c5ccccc5)c4)n3)cc2)cc1 ; CC(C)(C)c1ccc2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

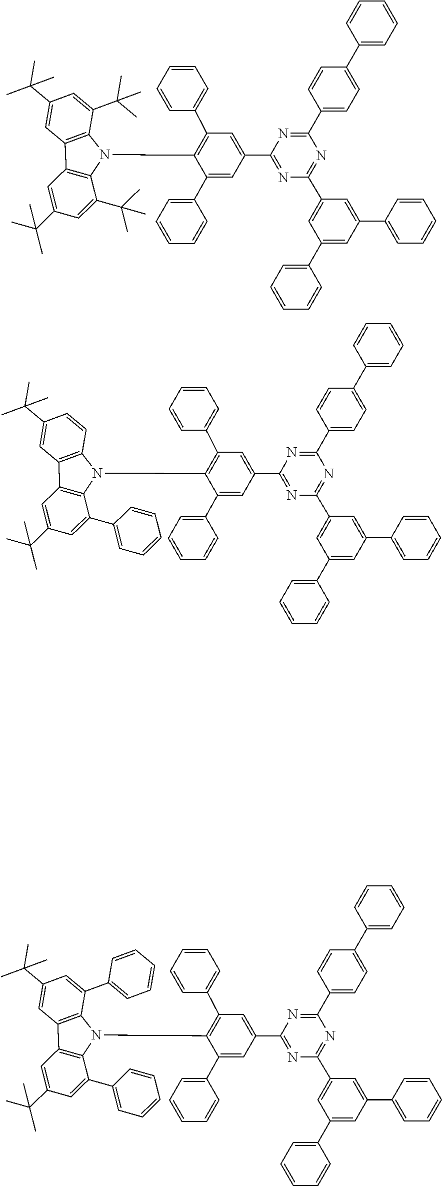 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

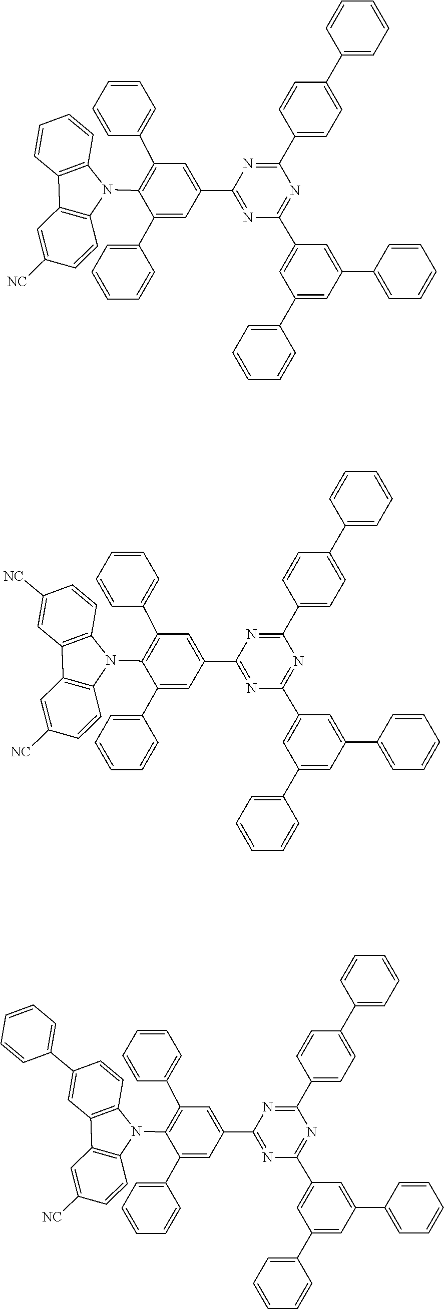 [SMILES: N#Cc1ccc2c(c1)c1ccccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; N#Cc1ccc2c(c1)c1cc(C#N)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; N#Cc1ccc2c(c1)c1cc(-c3ccccc3)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

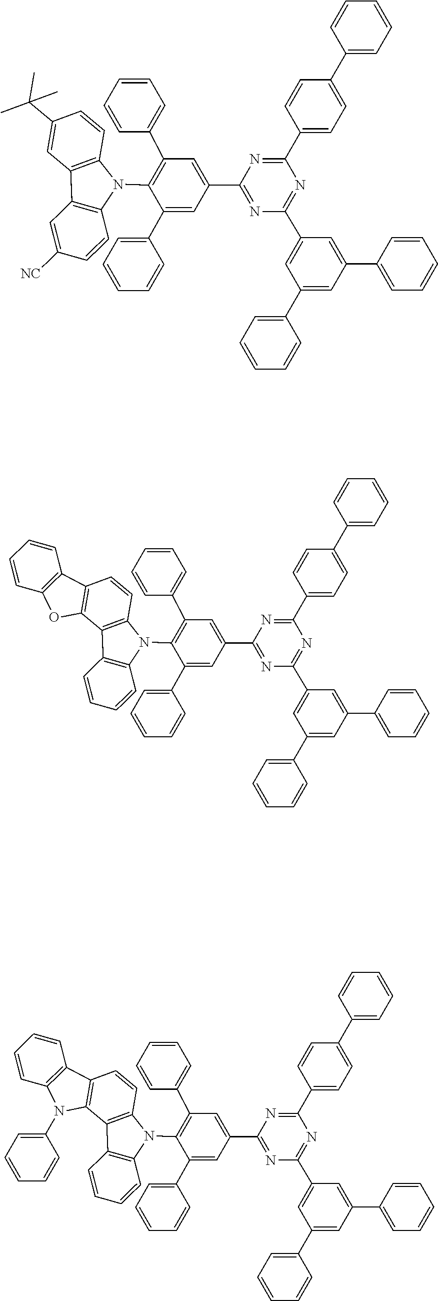 [SMILES: CC(C)(C)c1ccc2c(c1)c1cc(C#N)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8ccccc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccccc7n(-c7ccccc7)c56)c(-c5ccccc5)c4)n3)cc2)cc1]

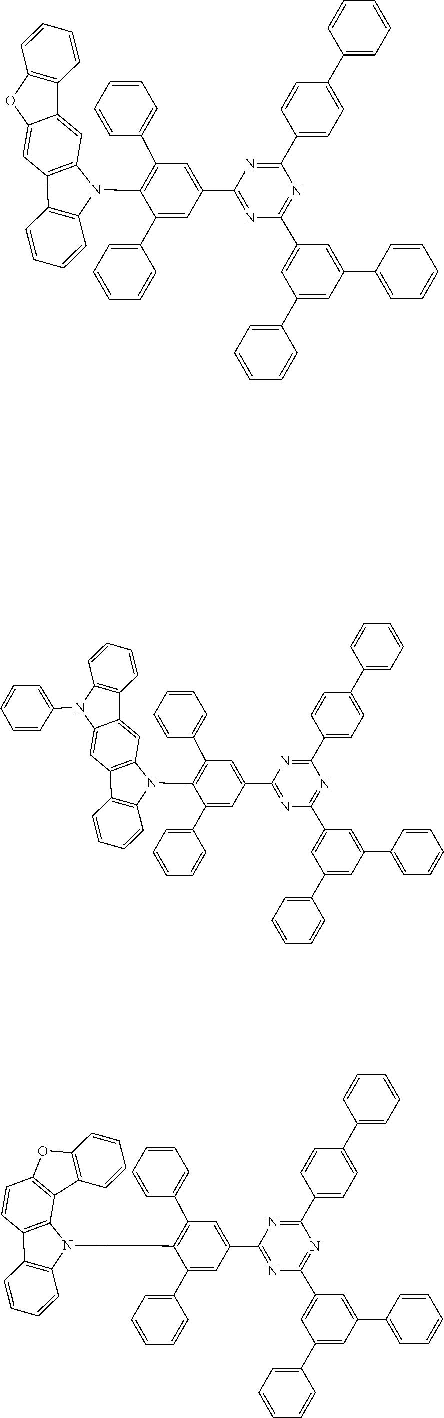 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc7oc8ccccc8c7cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)c5ccccc5n7-c5ccccc5)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7oc8ccccc8c7c65)c(-c5ccccc5)c4)n3)cc2)cc1]

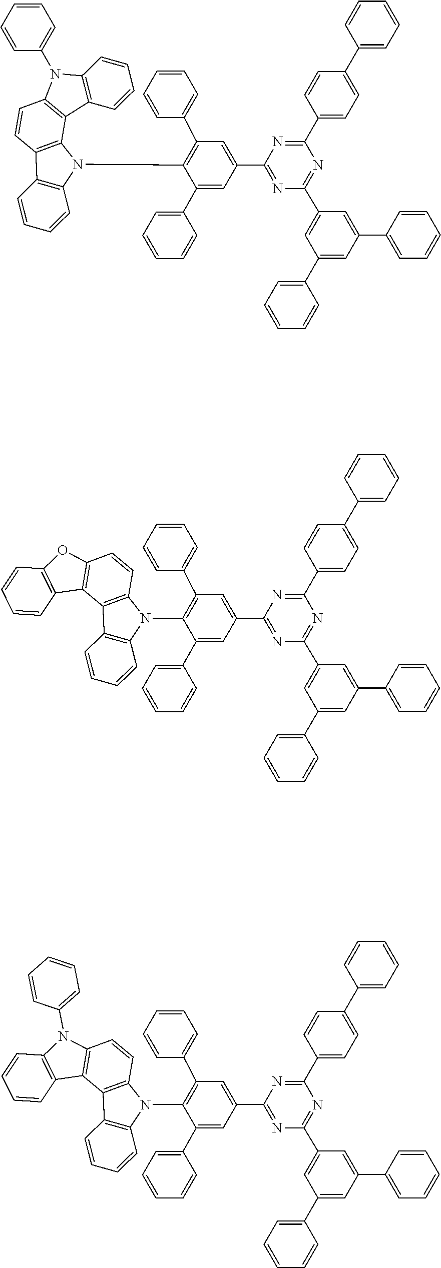 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc7c(c8ccccc8n7-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7c(ccc65)oc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7c8ccccc8n(-c8ccccc8)c7ccc65)c(-c5ccccc5)c4)n3)cc2)cc1]

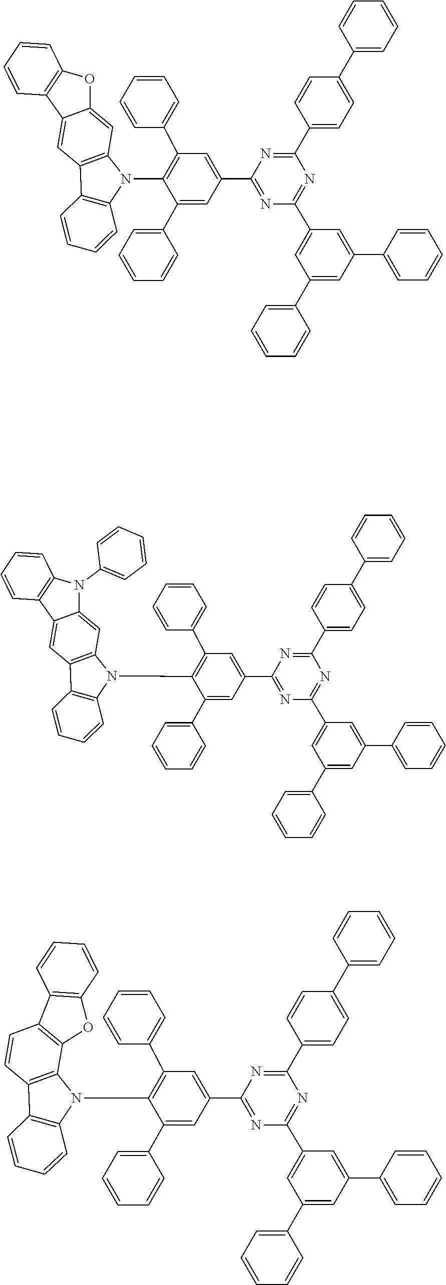 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)oc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7c8ccccc8n(-c8ccccc8)c7cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8oc7c65)c(-c5ccccc5)c4)n3)cc2)cc1]

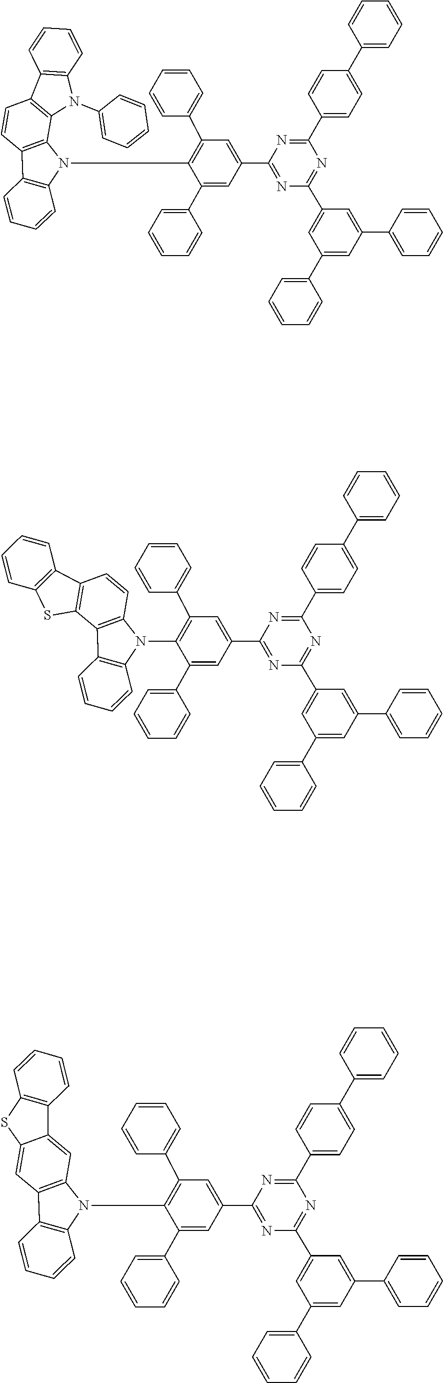 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8n(-c8ccccc8)c7c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8ccccc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7sc8ccccc8c7cc65)c(-c5ccccc5)c4)n3)cc2)cc1]

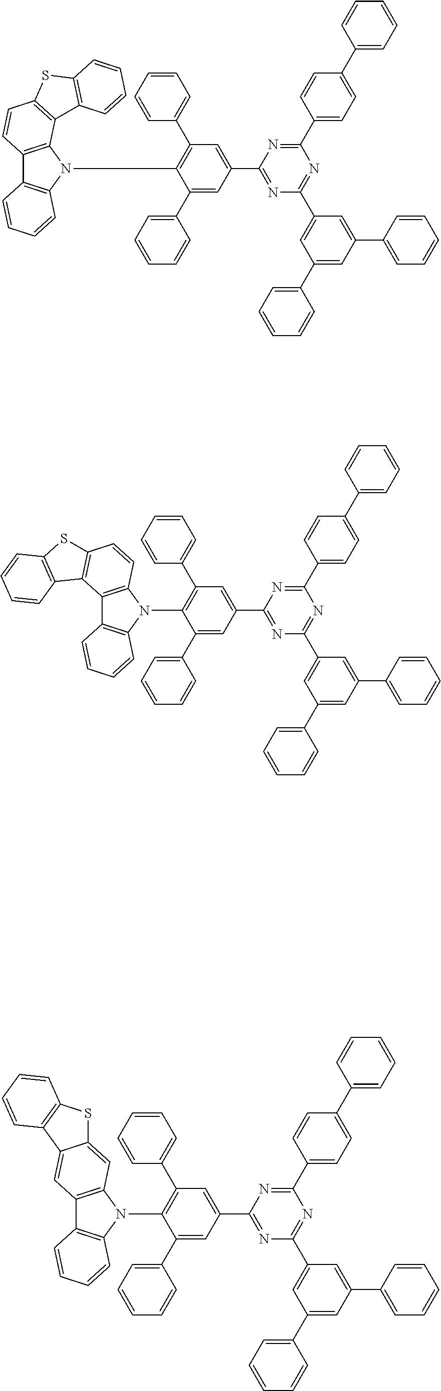 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc7sc8ccccc8c7c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7c(ccc65)sc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)sc5ccccc57)c(-c5ccccc5)c4)n3)cc2)cc1]

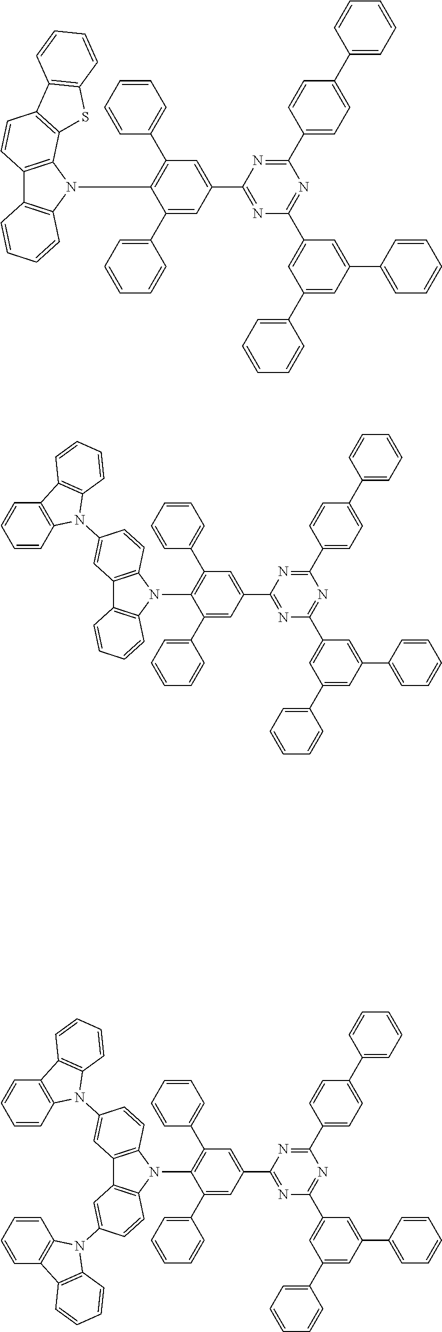 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8sc7c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc(-n7c8ccccc8c8ccccc87)ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1]

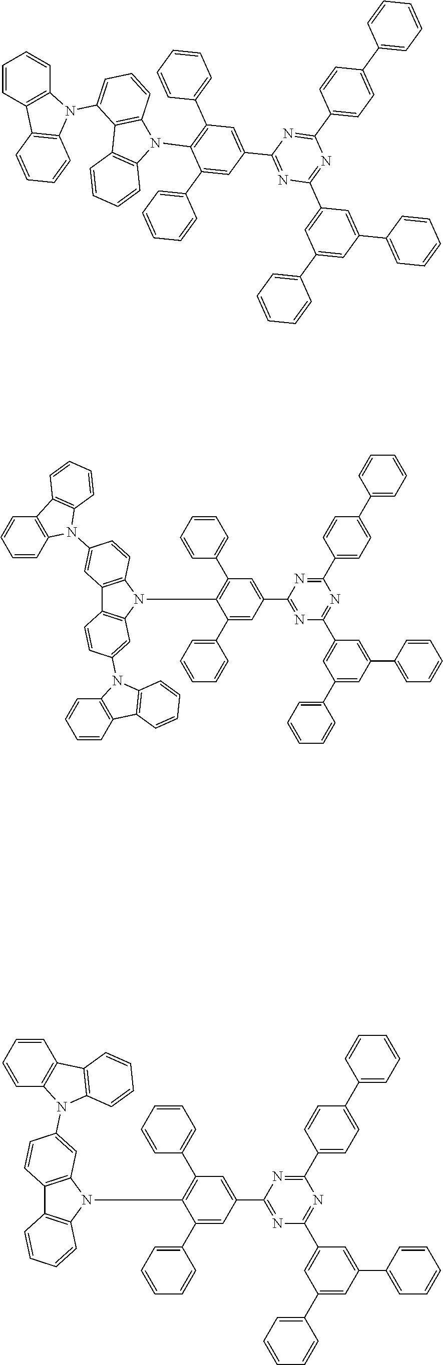 [SMILES: c1ccc(-c2ccc(-c3nc(-c4cc(-c5ccccc5)cc(-c5ccccc5)c4)nc(-c4cc(-c5ccccc5)c(-n5c6ccccc6c6c(-n7c8ccccc8c8ccccc87)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc(-n7c8ccccc8c8ccccc87)cc65)c(-c5ccccc5)c4)n3)cc2)cc1]

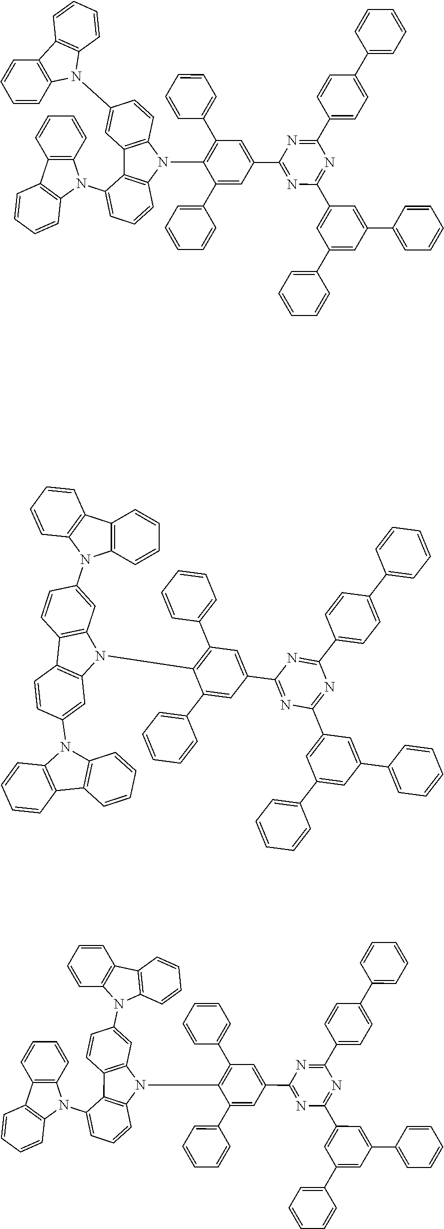 [SMILES: c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-n7c8ccccc8c8ccccc87)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6c(-n7c8ccccc8c8ccccc87)cccc65)c(-c5ccccc5)c4)n3)cc2)cc1]

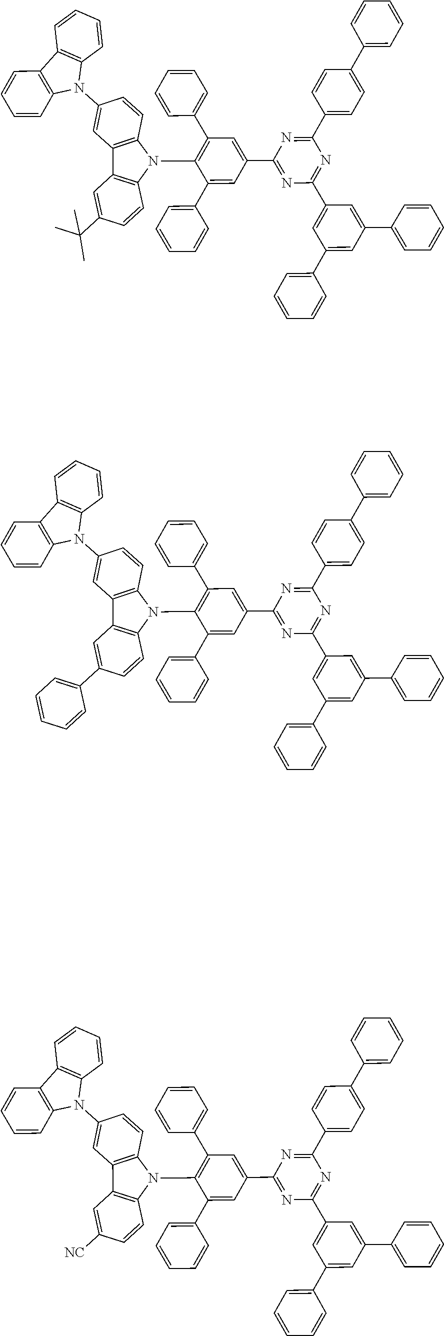 [SMILES: CC(C)(C)c1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-n7c8ccccc8c8ccccc87)ccc65)c(-c5ccccc5)c4)n3)cc2)cc1 ; N#Cc1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

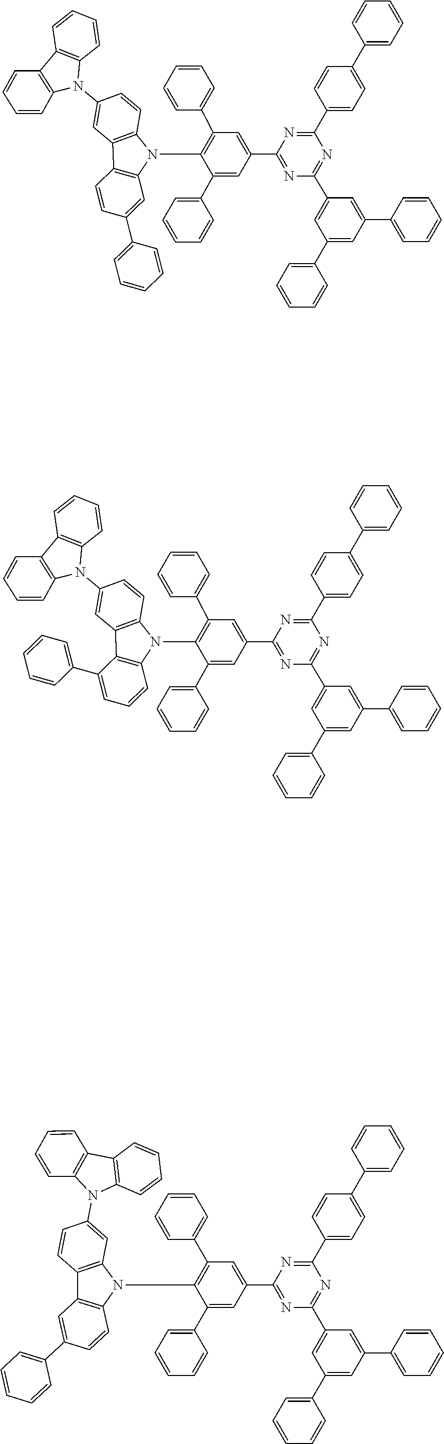 [SMILES: c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-c7ccccc7)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6ccc(-n7c8ccccc8c8ccccc87)cc65)c(-c5ccccc5)c4)n3)cc2)cc1]

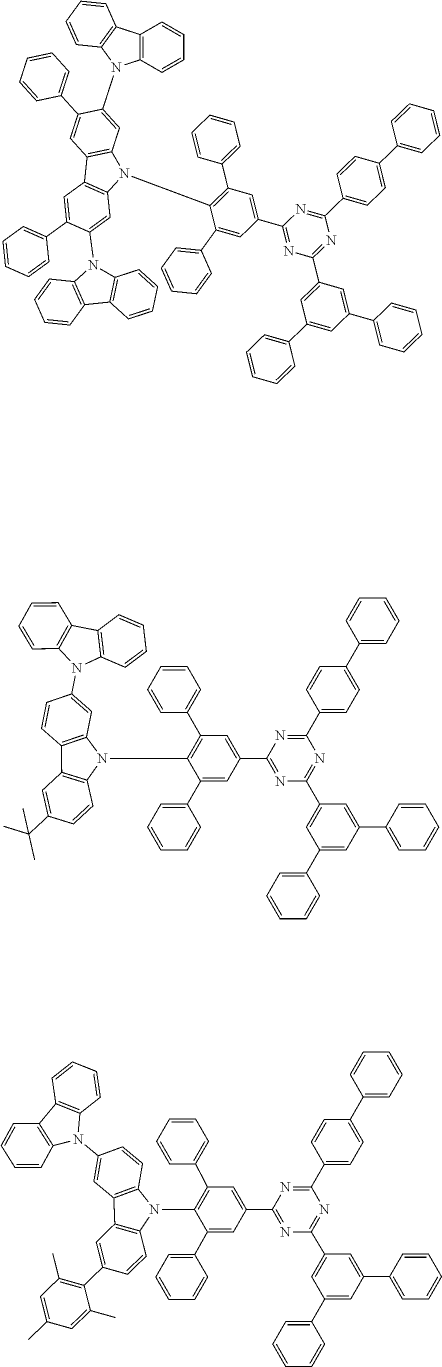 [SMILES: c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)c(-c7ccccc7)cc6c6cc(-c7ccccc7)c(-n7c8ccccc8c8ccccc87)cc65)c(-c5ccccc5)c4)n3)cc2)cc1 ; CC(C)(C)c1ccc2c(c1)c1ccc(-n3c4ccccc4c4ccccc43)cc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1]

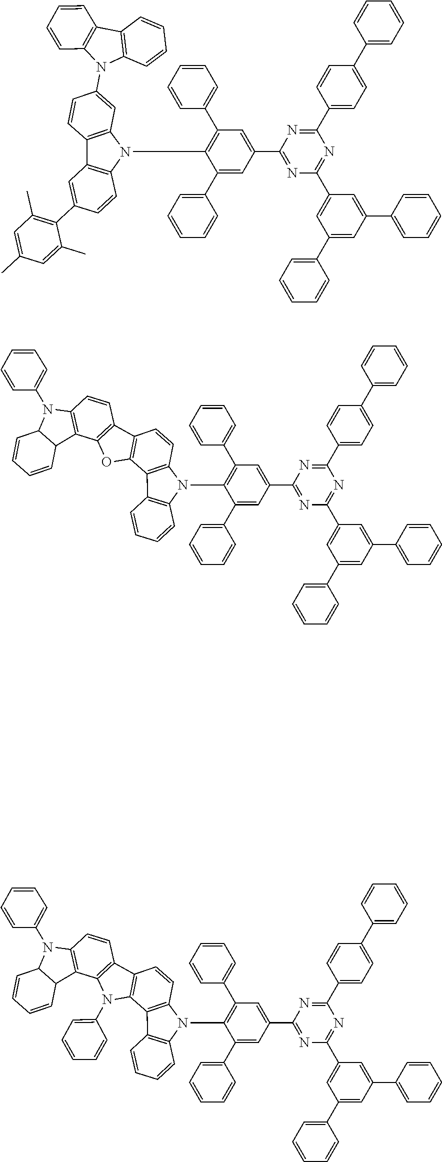 [SMILES: Cc1cc(C)c(-c2ccc3c(c2)c2ccc(-n4c5ccccc5c5ccccc54)cc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1 ; C1=CC2c3c(ccc4c3oc3c4ccc4c3c3ccccc3n4-c3c(-c4ccccc4)cc(-c4nc(-c5ccc(-c6ccccc6)cc5)nc(-c5cc(-c6ccccc6)cc(-c6ccccc6)c5)n4)cc3-c3ccccc3)N(c3ccccc3)C2C=C1 ; C1=CC2c3c(ccc4c5ccc6c(c7ccccc7n6-c6c(-c7ccccc7)cc(-c7nc(-c8ccc(-c9ccccc9)cc8)nc(-c8cc(-c9ccccc9)cc(-c9ccccc9)c8)n7)cc6-c6ccccc6)c5n(-c5ccccc5)c34)N(c3ccccc3)C2C=C1]

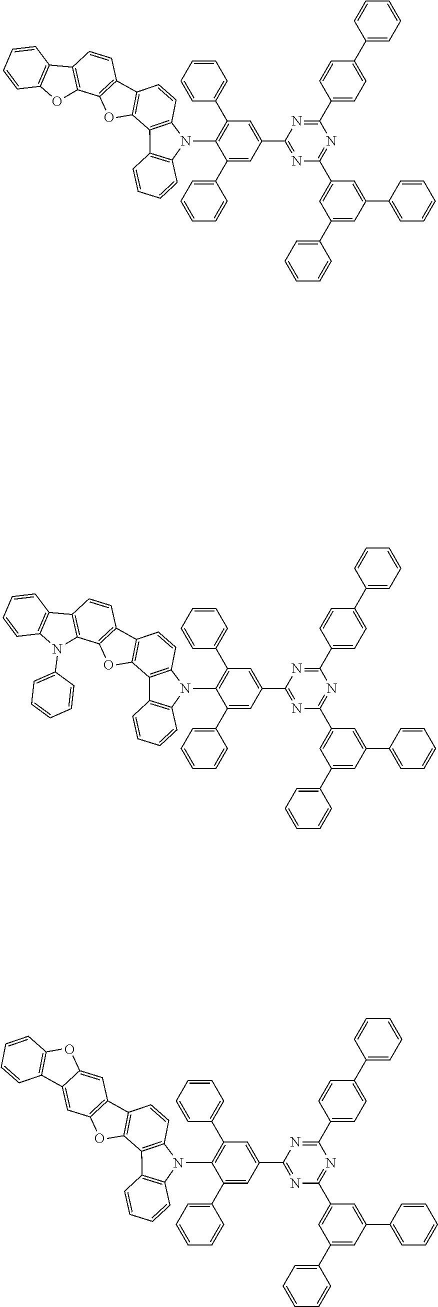 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10oc98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10n(-c%10ccccc%10)c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)oc5ccccc59)c(-c5ccccc5)c4)n3)cc2)cc1]

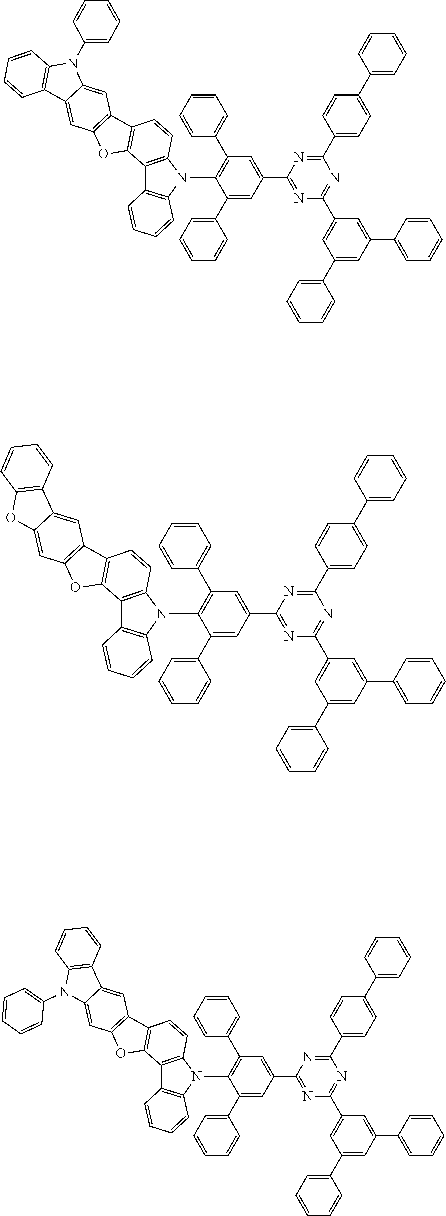 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c%10ccccc%10n(-c%10ccccc%10)c9cc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9oc%10ccccc%10c9cc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)c5ccccc5n9-c5ccccc5)c(-c5ccccc5)c4)n3)cc2)cc1]

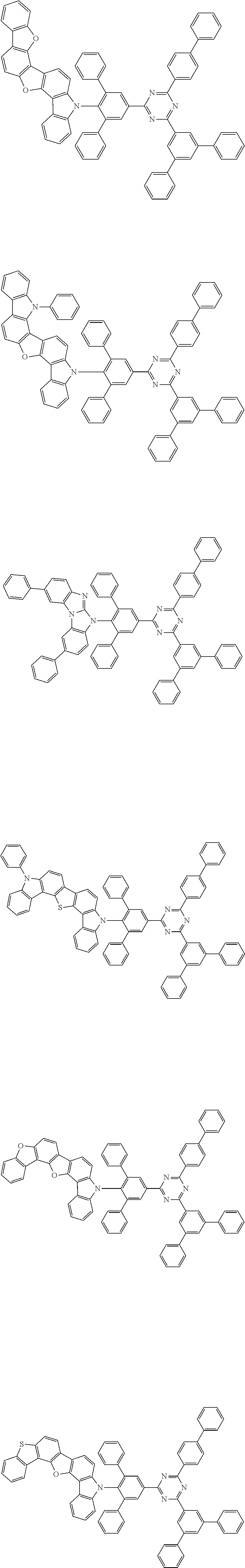

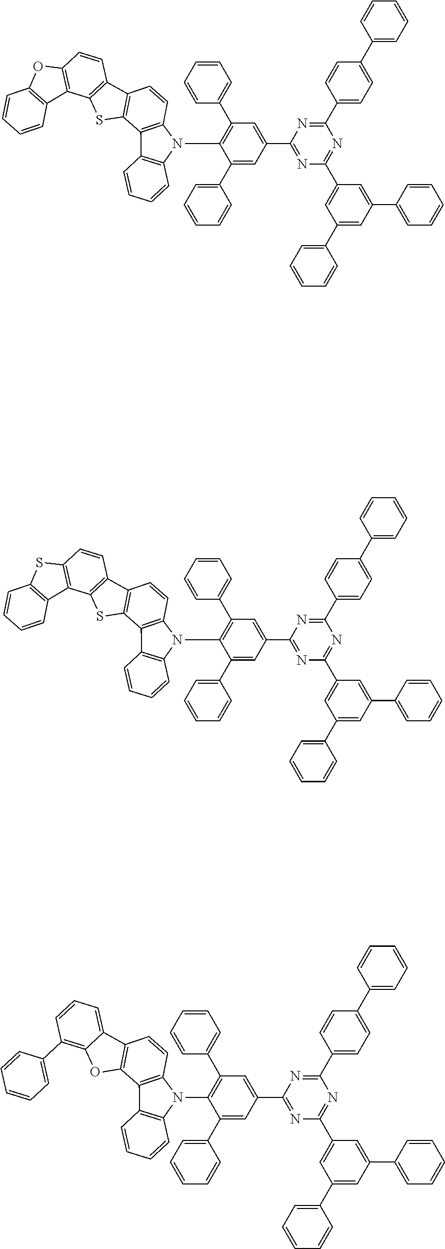 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9oc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9sc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(-c9ccccc9)cccc8c7ccc65)c(-c5ccccc5)c4)n3)cc2)cc1]

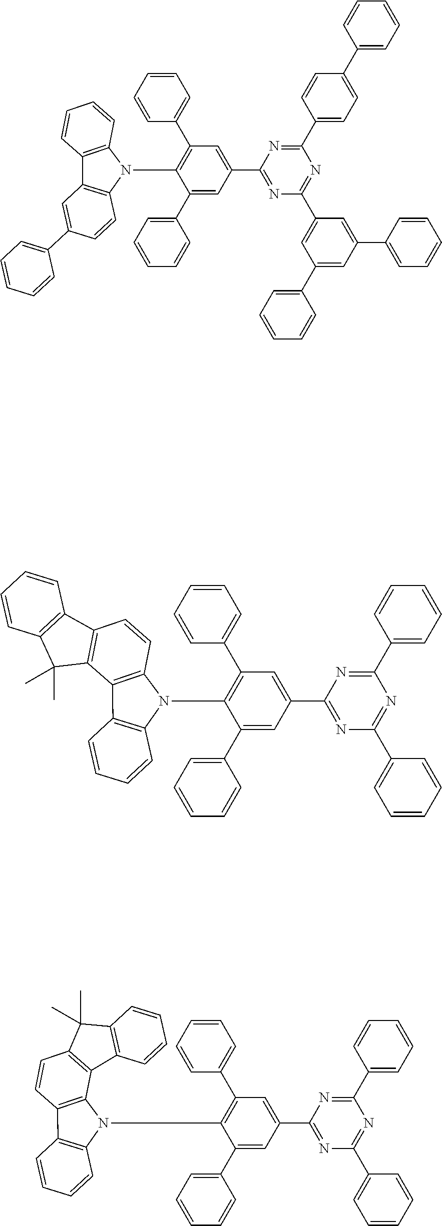 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc(-c7ccccc7)ccc65)c(-c5ccccc5)c4)n3)cc2)cc1 ; CC1(C)c2ccccc2-c2ccc3c(c21)c1ccccc1n3-c1c(-c2ccccc2)cc(-c2nc(-c3ccccc3)nc(-c3ccccc3)n2)cc1-c1ccccc1 ; CC1(C)c2ccccc2-c2c1ccc1c3ccccc3n(-c3c(-c4ccccc4)cc(-c4nc(-c5ccccc5)nc(-c5ccccc5)n4)cc3-c3ccccc3)c21]

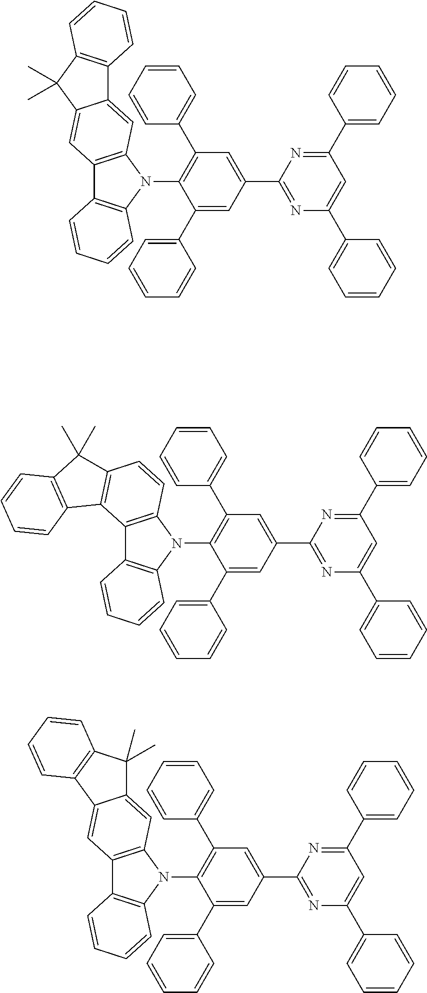 [SMILES: CC1(C)c2ccccc2-c2cc3c(cc21)c1ccccc1n3-c1c(-c2ccccc2)cc(-c2nc(-c3ccccc3)cc(-c3ccccc3)n2)cc1-c1ccccc1 ; CC1(C)c2ccccc2-c2c1ccc1c2c2ccccc2n1-c1c(-c2ccccc2)cc(-c2nc(-c3ccccc3)cc(-c3ccccc3)n2)cc1-c1ccccc1 ; CC1(C)c2ccccc2-c2cc3c4ccccc4n(-c4c(-c5ccccc5)cc(-c5nc(-c6ccccc6)cc(-c6ccccc6)n5)cc4-c4ccccc4)c3cc21]

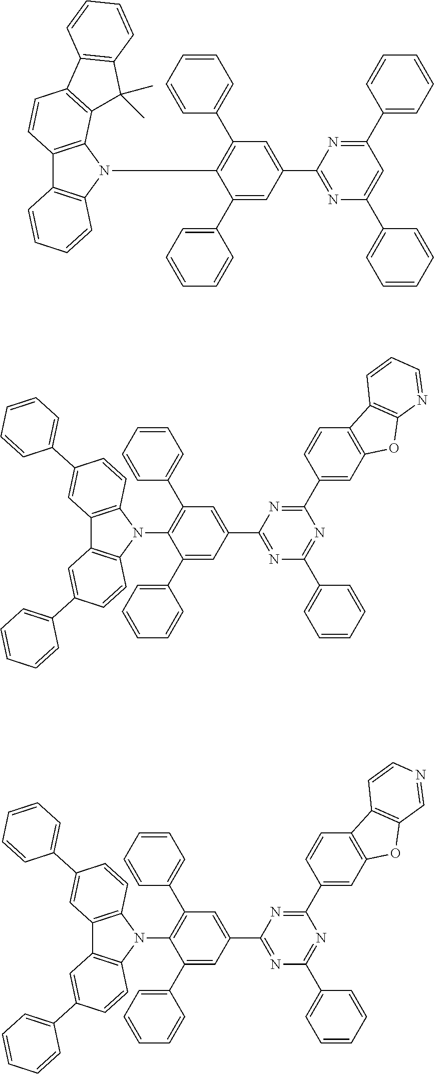 [SMILES: CC1(C)c2ccccc2-c2ccc3c4ccccc4n(-c4c(-c5ccccc5)cc(-c5nc(-c6ccccc6)cc(-c6ccccc6)n5)cc4-c4ccccc4)c3c21 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4ncccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4cnccc45)n3)cc2-c2ccccc2)cc1]

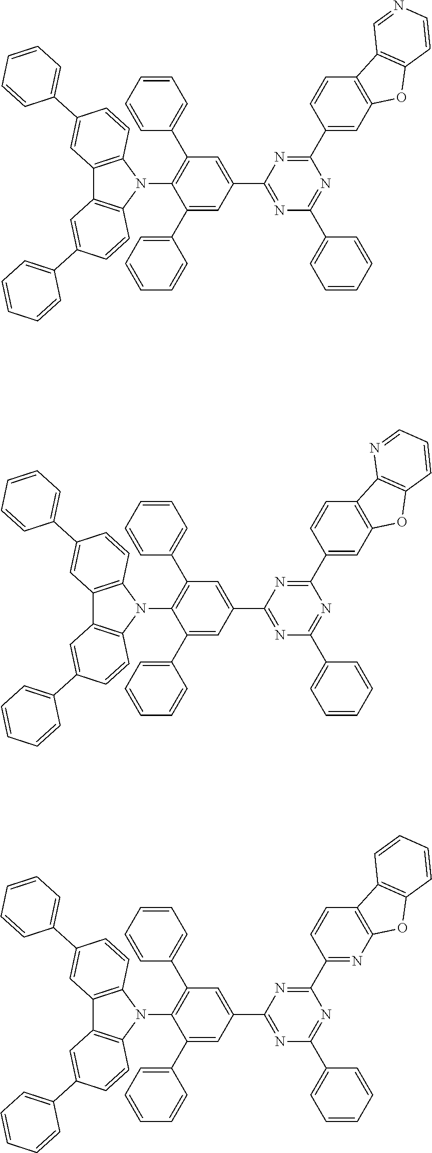 [SMILES: c1ccc(-c2ccc3c(c2)c2cc(-c4ccccc4)ccc2n3-c2c(-c3ccccc3)cc(-c3nc(-c4ccccc4)nc(-c4ccc5c(c4)oc4ccncc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4cccnc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(n4)oc4ccccc45)n3)cc2-c2ccccc2)cc1]

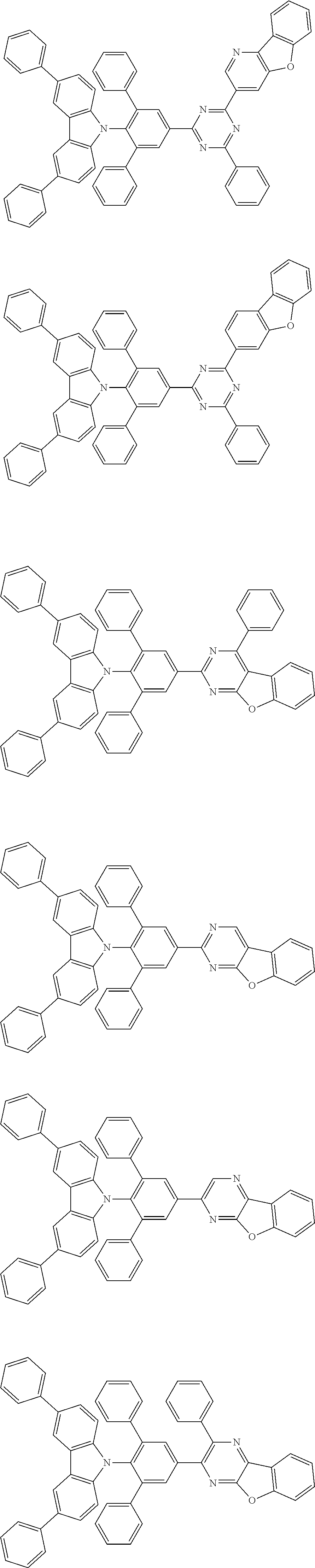

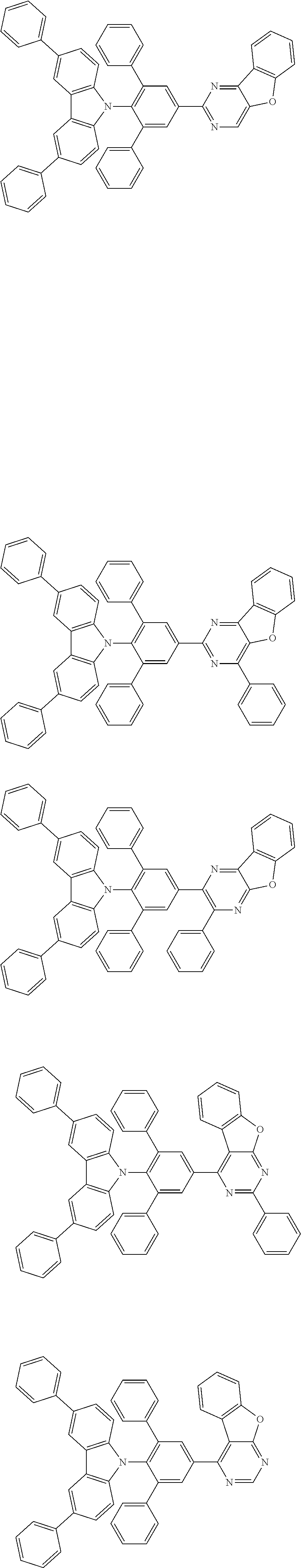

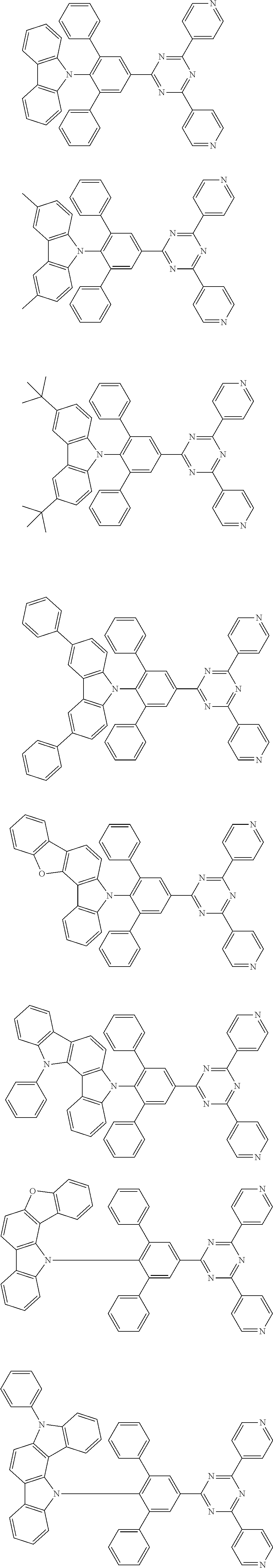

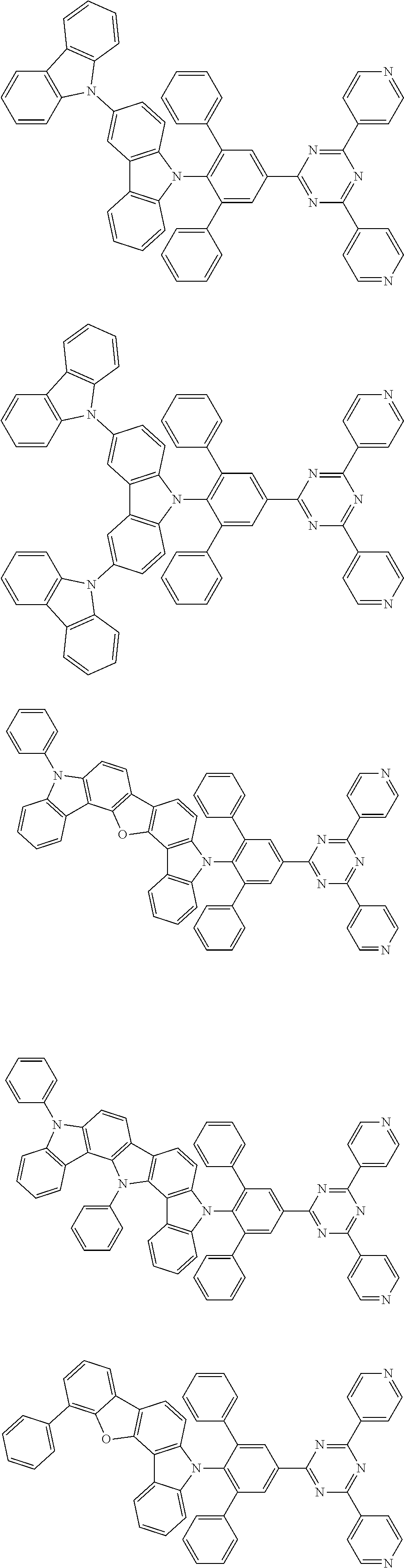

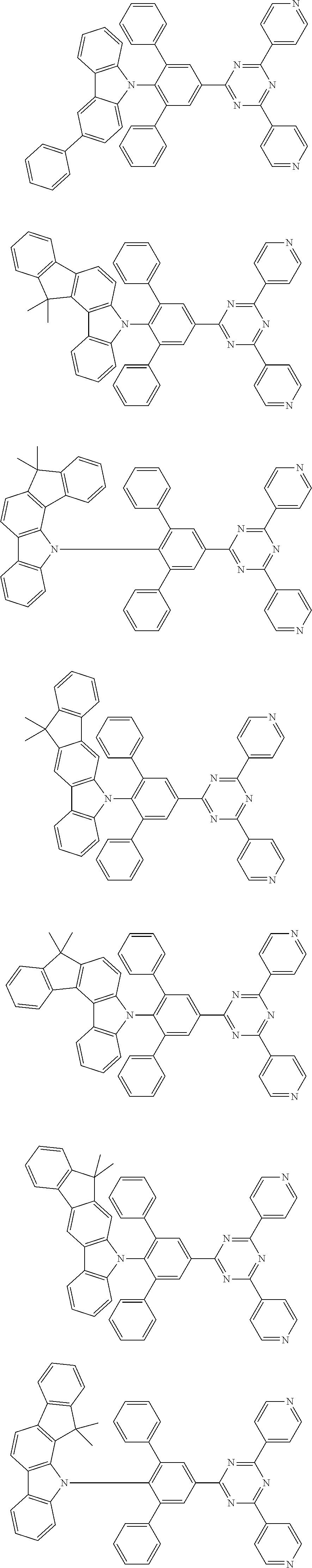

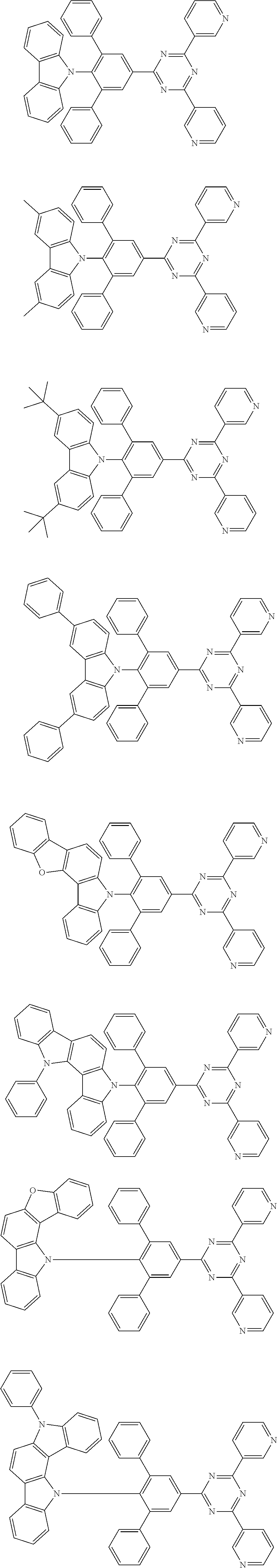

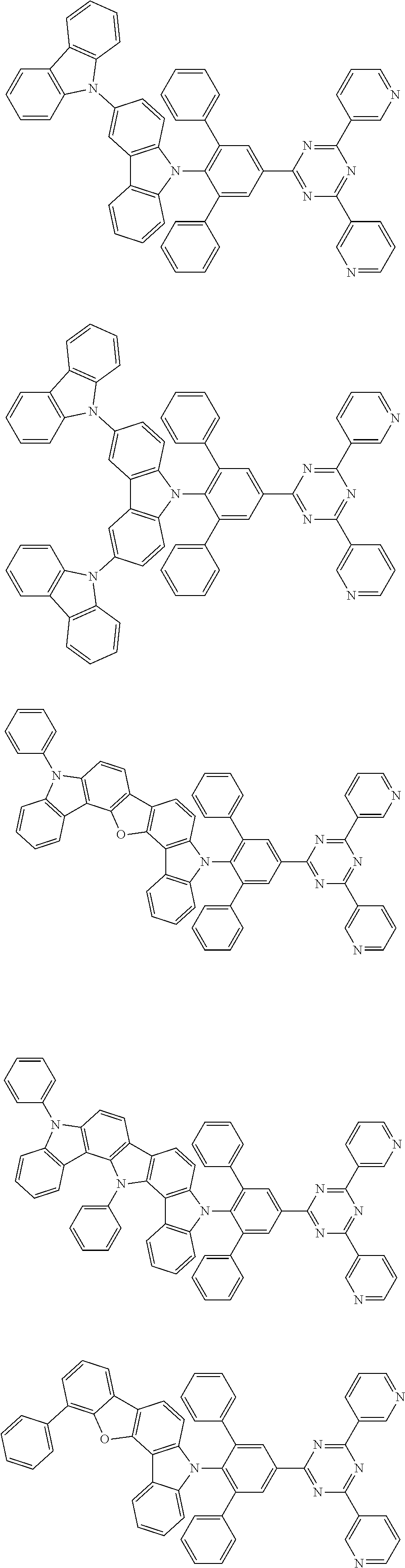

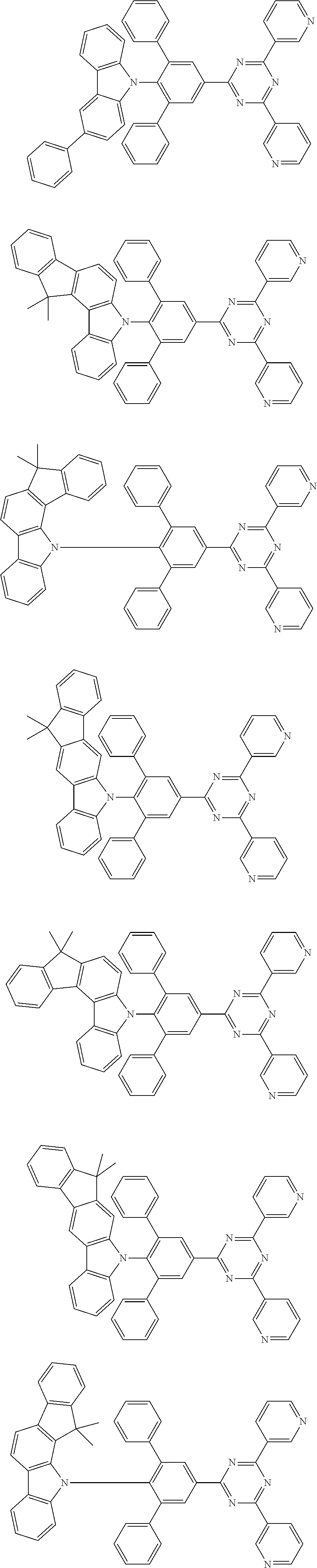

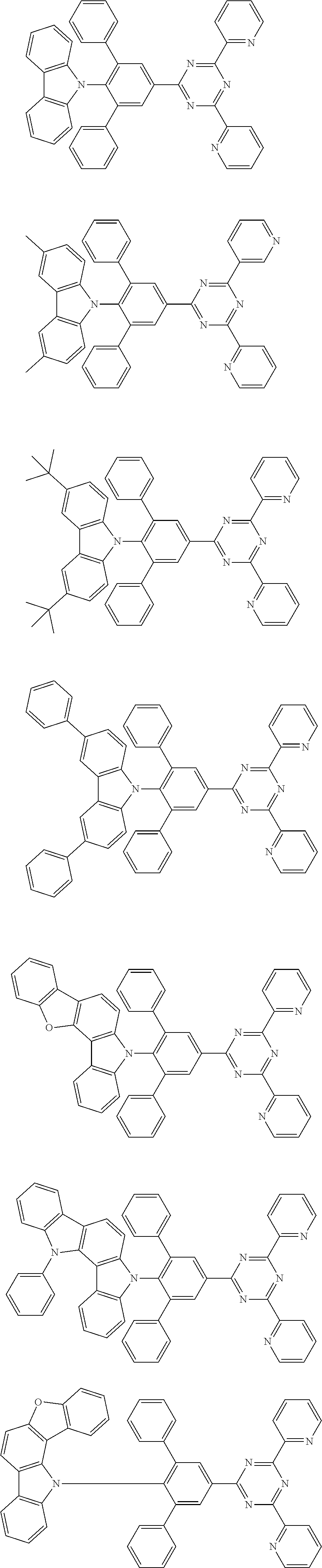

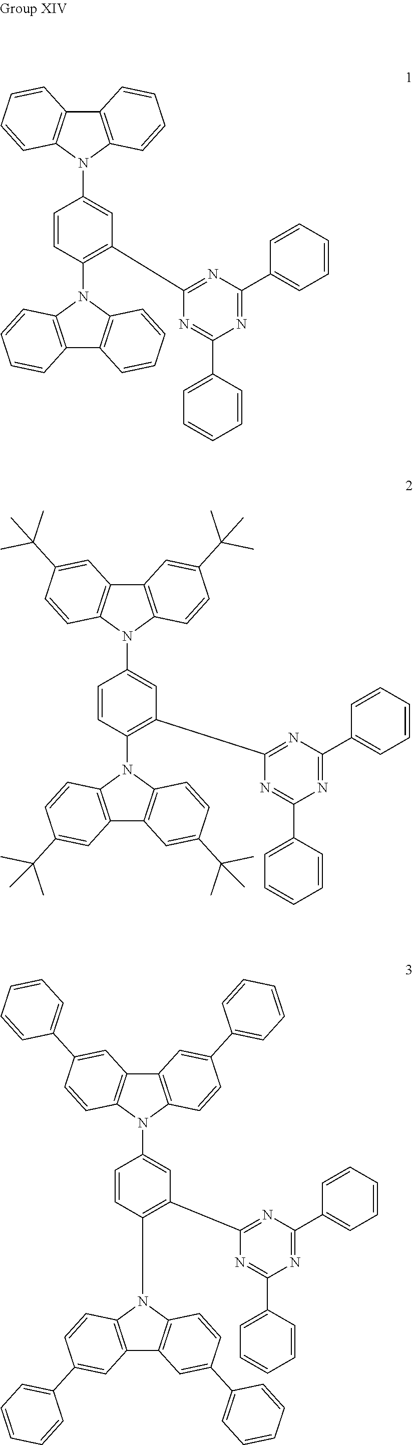 [SMILES: c1ccc(-c2nc(-c3ccccc3)nc(-c3cc(-n4c5ccccc5c5ccccc54)ccc3-n3c4ccccc4c4ccccc43)n2)cc1 ; CC(C)(C)c1ccc2c(c1)c1cc(C(C)(C)C)ccc1n2-c1ccc(-n2c3ccc(C(C)(C)C)cc3c3cc(C(C)(C)C)ccc32)c(-c2nc(-c3ccccc3)nc(-c3ccccc3)n2)c1 ; c1ccc(-c2ccc3c(c2)c2cc(-c4ccccc4)ccc2n3-c2ccc(-n3c4ccc(-c5ccccc5)cc4c4cc(-c5ccccc5)ccc43)c(-c3nc(-c4ccccc4)nc(-c4ccccc4)n3)c2)cc1]

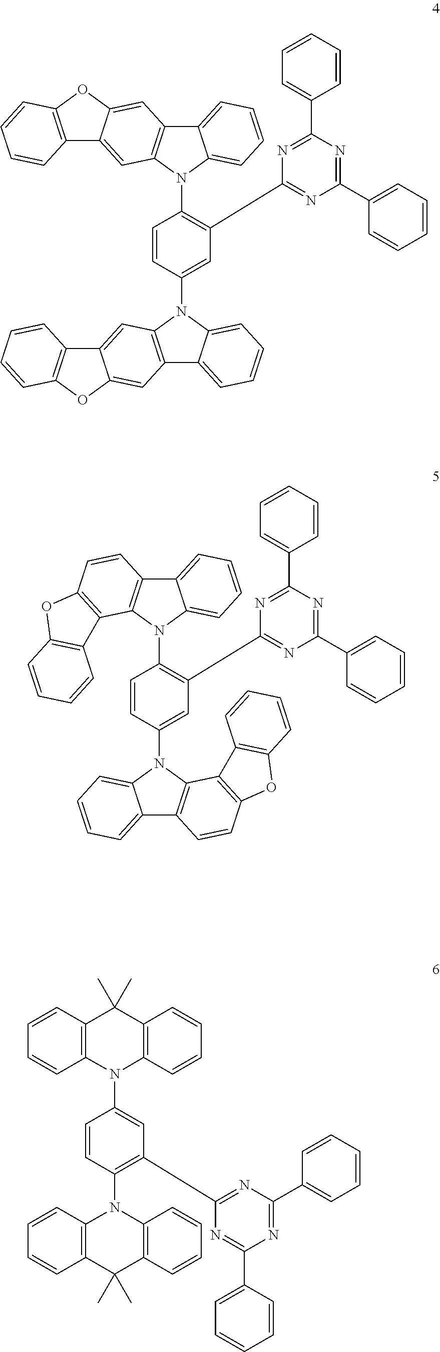 [SMILES: c1ccc(-c2nc(-c3ccccc3)nc(-c3cc(-n4c5ccccc5c5cc6oc7ccccc7c6cc54)ccc3-n3c4ccccc4c4cc5oc6ccccc6c5cc43)n2)cc1 ; c1ccc(-c2nc(-c3ccccc3)nc(-c3cc(-n4c5ccccc5c5ccc6oc7ccccc7c6c54)ccc3-n3c4ccccc4c4ccc5oc6ccccc6c5c43)n2)cc1 ; CC1(C)c2ccccc2N(c2ccc(N3c4ccccc4C(C)(C)c4ccccc43)c(-c3nc(-c4ccccc4)nc(-c4ccccc4)n3)c2)c2ccccc21]

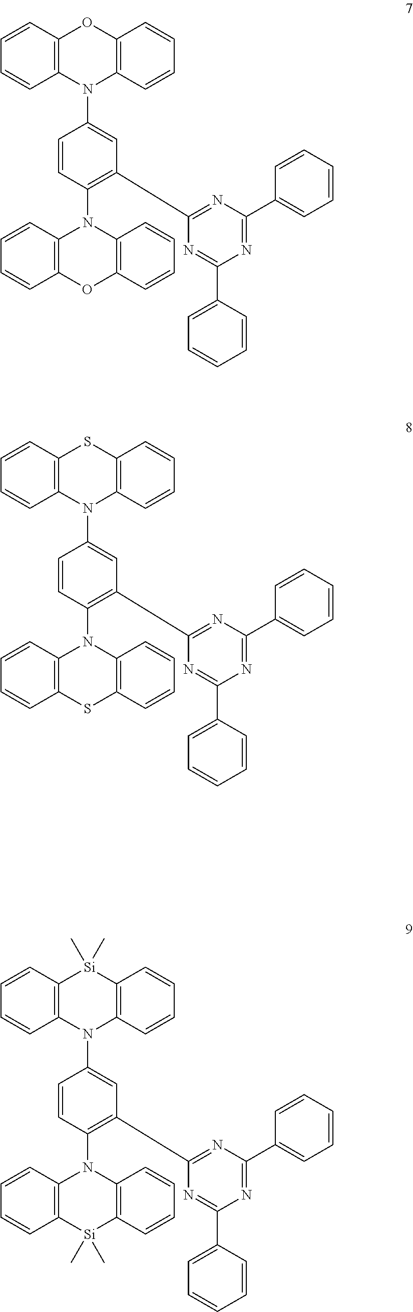 [SMILES: c1ccc(-c2nc(-c3ccccc3)nc(-c3cc(N4c5ccccc5Oc5ccccc54)ccc3N3c4ccccc4Oc4ccccc43)n2)cc1 ; c1ccc(-c2nc(-c3ccccc3)nc(-c3cc(N4c5ccccc5Sc5ccccc54)ccc3N3c4ccccc4Sc4ccccc43)n2)cc1 ; C[Si]1(C)c2ccccc2N(c2ccc(N3c4ccccc4[Si](C)(C)c4ccccc43)c(-c3nc(-c4ccccc4)nc(-c4ccccc4)n3)c2)c2ccccc21]

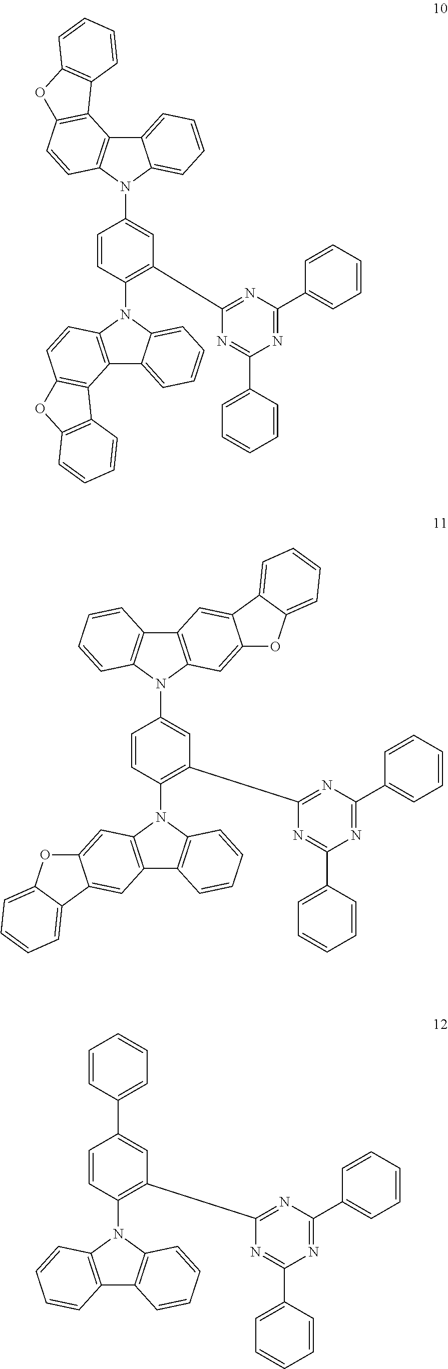 [SMILES: c1ccc(-c2nc(-c3ccccc3)nc(-c3cc(-n4c5ccccc5c5c6c(ccc54)oc4ccccc46)ccc3-n3c4ccccc4c4c5c(ccc43)oc3ccccc35)n2)cc1 ; c1ccc(-c2nc(-c3ccccc3)nc(-c3cc(-n4c5ccccc5c5cc6c(cc54)oc4ccccc46)ccc3-n3c4ccccc4c4cc5c(cc43)oc3ccccc35)n2)cc1 ; c1ccc(-c2ccc(-n3c4ccccc4c4ccccc43)c(-c3nc(-c4ccccc4)nc(-c4ccccc4)n3)c2)cc1]

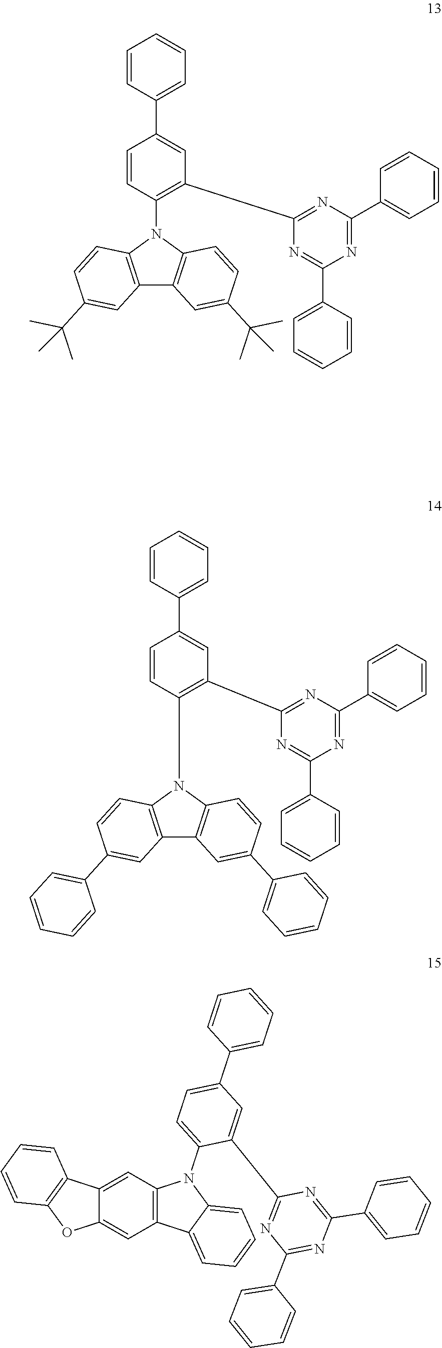 [SMILES: CC(C)(C)c1ccc2c(c1)c1cc(C(C)(C)C)ccc1n2-c1ccc(-c2ccccc2)cc1-c1nc(-c2ccccc2)nc(-c2ccccc2)n1 ; c1ccc(-c2ccc(-n3c4ccc(-c5ccccc5)cc4c4cc(-c5ccccc5)ccc43)c(-c3nc(-c4ccccc4)nc(-c4ccccc4)n3)c2)cc1 ; c1ccc(-c2ccc(-n3c4ccccc4c4cc5oc6ccccc6c5cc43)c(-c3nc(-c4ccccc4)nc(-c4ccccc4)n3)c2)cc1]

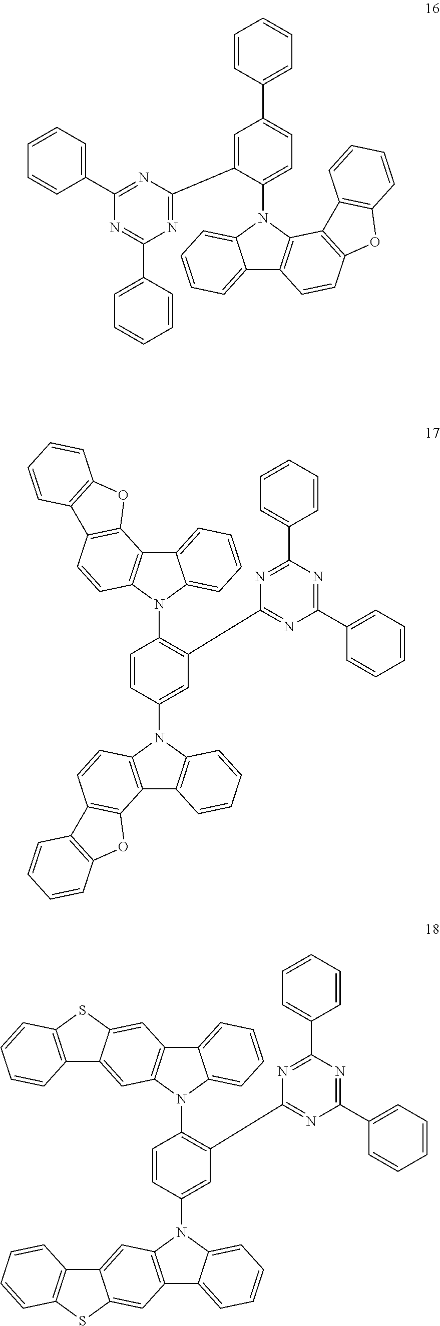 [SMILES: c1ccc(-c2ccc(-n3c4ccccc4c4ccc5oc6ccccc6c5c43)c(-c3nc(-c4ccccc4)nc(-c4ccccc4)n3)c2)cc1 ; c1ccc(-c2nc(-c3ccccc3)nc(-c3cc(-n4c5ccccc5c5c6oc7ccccc7c6ccc54)ccc3-n3c4ccccc4c4c5oc6ccccc6c5ccc43)n2)cc1 ; c1ccc(-c2nc(-c3ccccc3)nc(-c3cc(-n4c5ccccc5c5cc6sc7ccccc7c6cc54)ccc3-n3c4ccccc4c4cc5sc6ccccc6c5cc43)n2)cc1]

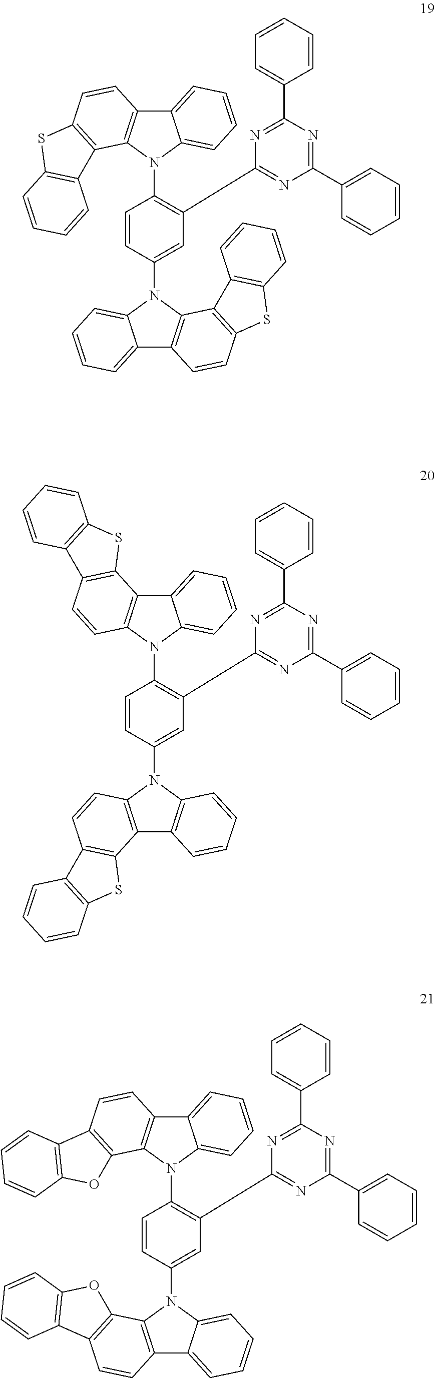 [SMILES: c1ccc(-c2nc(-c3ccccc3)nc(-c3cc(-n4c5ccccc5c5ccc6sc7ccccc7c6c54)ccc3-n3c4ccccc4c4ccc5sc6ccccc6c5c43)n2)cc1 ; c1ccc(-c2nc(-c3ccccc3)nc(-c3cc(-n4c5ccccc5c5c6sc7ccccc7c6ccc54)ccc3-n3c4ccccc4c4c5sc6ccccc6c5ccc43)n2)cc1 ; c1ccc(-c2nc(-c3ccccc3)nc(-c3cc(-n4c5ccccc5c5ccc6c7ccccc7oc6c54)ccc3-n3c4ccccc4c4ccc5c6ccccc6oc5c43)n2)cc1]

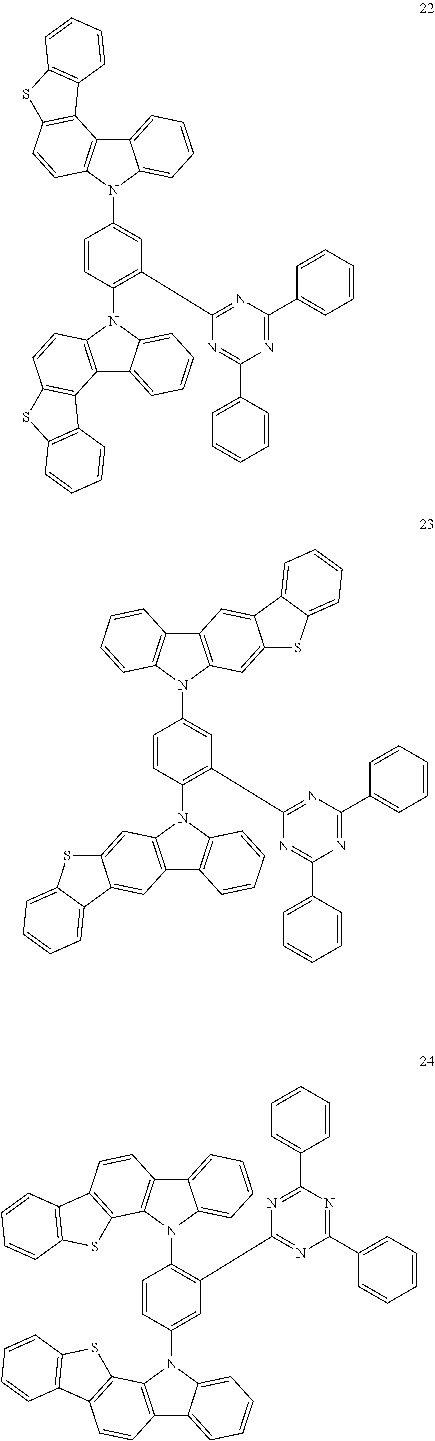 [SMILES: c1ccc(-c2nc(-c3ccccc3)nc(-c3cc(-n4c5ccccc5c5c6c(ccc54)sc4ccccc46)ccc3-n3c4ccccc4c4c5c(ccc43)sc3ccccc35)n2)cc1 ; c1ccc(-c2nc(-c3ccccc3)nc(-c3cc(-n4c5ccccc5c5cc6c(cc54)sc4ccccc46)ccc3-n3c4ccccc4c4cc5c(cc43)sc3ccccc35)n2)cc1 ; c1ccc(-c2nc(-c3ccccc3)nc(-c3cc(-n4c5ccccc5c5ccc6c7ccccc7sc6c54)ccc3-n3c4ccccc4c4ccc5c6ccccc6sc5c43)n2)cc1]

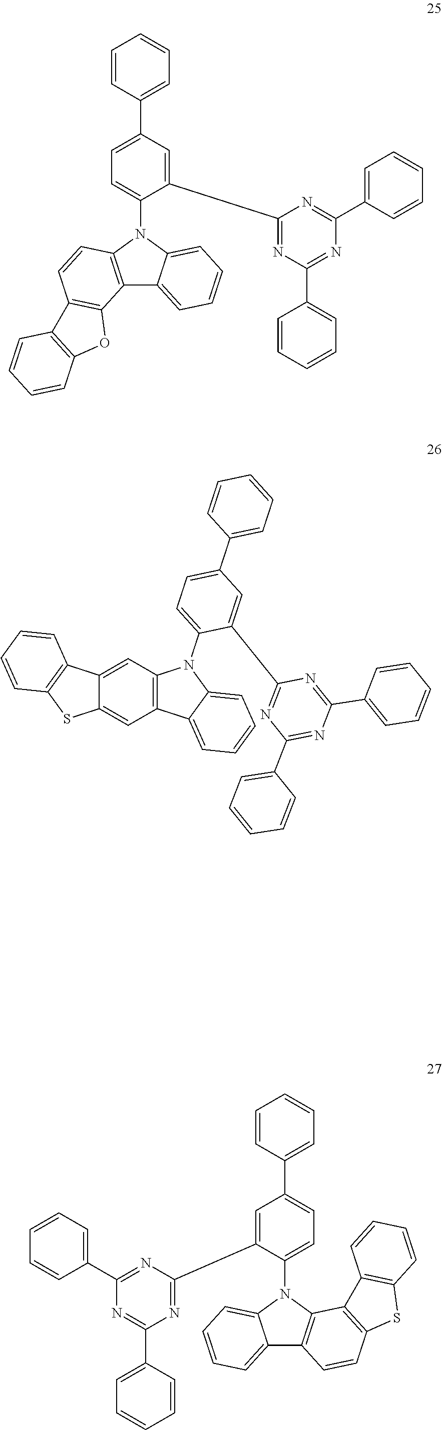 [SMILES: c1ccc(-c2ccc(-n3c4ccccc4c4c5oc6ccccc6c5ccc43)c(-c3nc(-c4ccccc4)nc(-c4ccccc4)n3)c2)cc1 ; c1ccc(-c2ccc(-n3c4ccccc4c4cc5sc6ccccc6c5cc43)c(-c3nc(-c4ccccc4)nc(-c4ccccc4)n3)c2)cc1 ; c1ccc(-c2ccc(-n3c4ccccc4c4ccc5sc6ccccc6c5c43)c(-c3nc(-c4ccccc4)nc(-c4ccccc4)n3)c2)cc1]

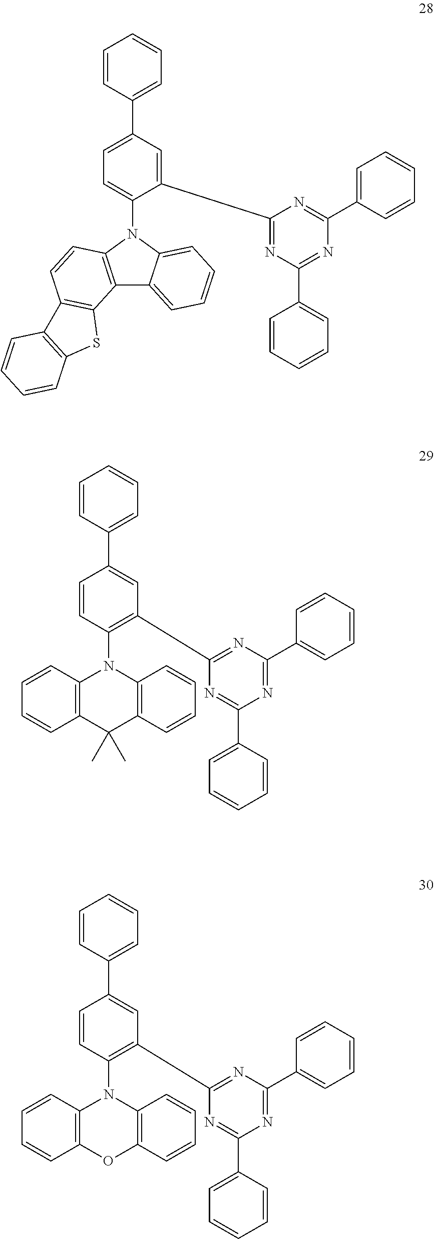 [SMILES: c1ccc(-c2ccc(-n3c4ccccc4c4c5sc6ccccc6c5ccc43)c(-c3nc(-c4ccccc4)nc(-c4ccccc4)n3)c2)cc1 ; CC1(C)c2ccccc2N(c2ccc(-c3ccccc3)cc2-c2nc(-c3ccccc3)nc(-c3ccccc3)n2)c2ccccc21 ; c1ccc(-c2ccc(N3c4ccccc4Oc4ccccc43)c(-c3nc(-c4ccccc4)nc(-c4ccccc4)n3)c2)cc1]

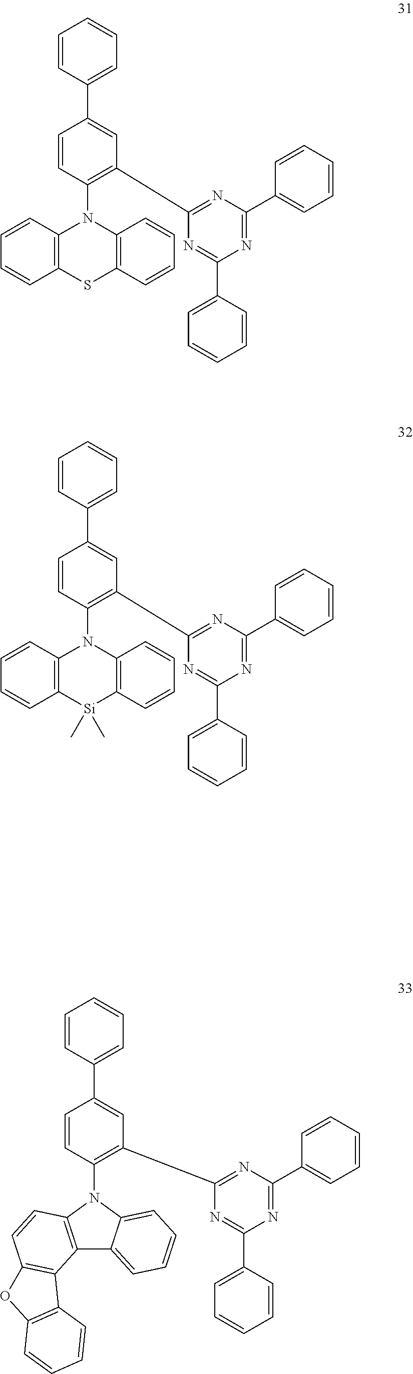 [SMILES: c1ccc(-c2ccc(N3c4ccccc4Sc4ccccc43)c(-c3nc(-c4ccccc4)nc(-c4ccccc4)n3)c2)cc1 ; C[Si]1(C)c2ccccc2N(c2ccc(-c3ccccc3)cc2-c2nc(-c3ccccc3)nc(-c3ccccc3)n2)c2ccccc21 ; c1ccc(-c2ccc(-n3c4ccccc4c4c5c(ccc43)oc3ccccc35)c(-c3nc(-c4ccccc4)nc(-c4ccccc4)n3)c2)cc1]

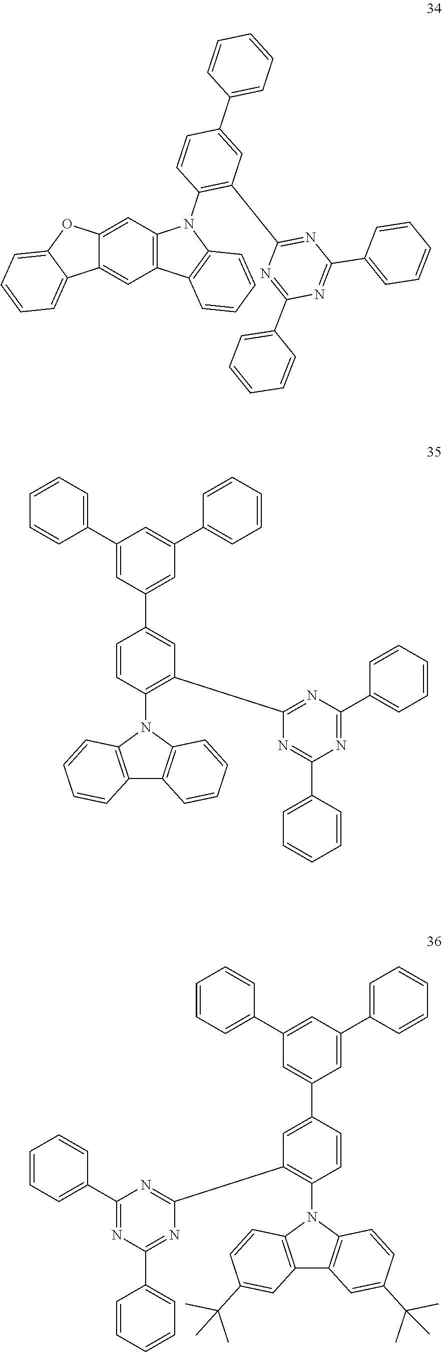 [SMILES: c1ccc(-c2ccc(-n3c4ccccc4c4cc5c(cc43)oc3ccccc35)c(-c3nc(-c4ccccc4)nc(-c4ccccc4)n3)c2)cc1 ; c1ccc(-c2cc(-c3ccccc3)cc(-c3ccc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ccc(-c2cc(-c3ccccc3)cc(-c3ccccc3)c2)cc1-c1nc(-c2ccccc2)nc(-c2ccccc2)n1]

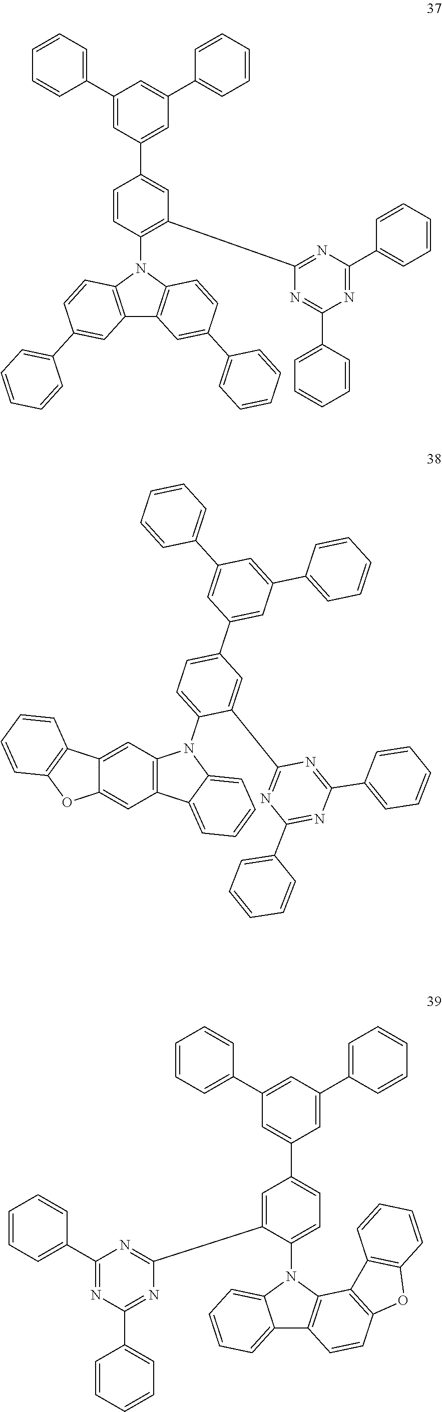 [SMILES: c1ccc(-c2cc(-c3ccccc3)cc(-c3ccc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5ccc6oc7ccccc7c6c54)c(-c4nc(-c5ccccc5)nc(-c5ccccc5)n4)c3)c2)cc1]

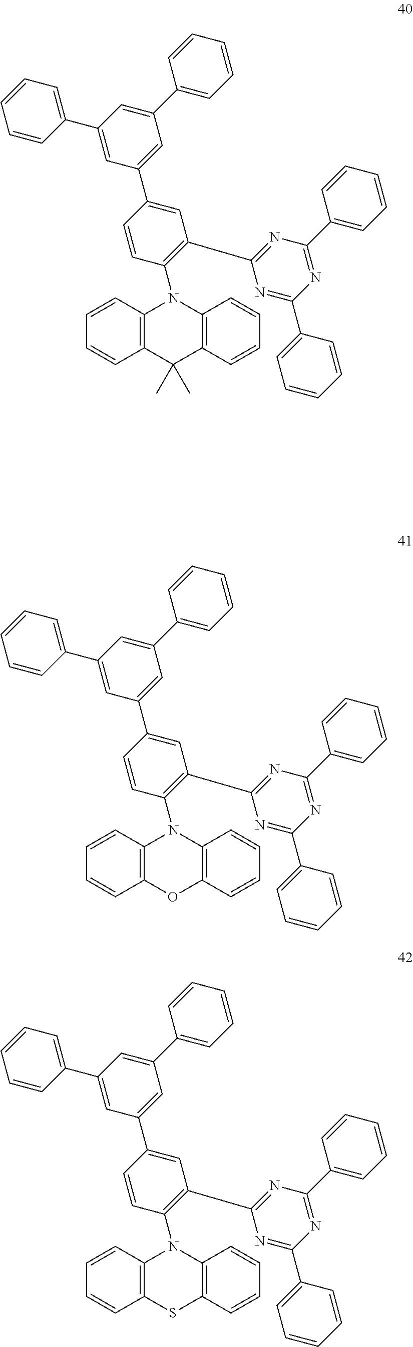 [SMILES: CC1(C)c2ccccc2N(c2ccc(-c3cc(-c4ccccc4)cc(-c4ccccc4)c3)cc2-c2nc(-c3ccccc3)nc(-c3ccccc3)n2)c2ccccc21 ; c1ccc(-c2cc(-c3ccccc3)cc(-c3ccc(N4c5ccccc5Oc5ccccc54)c(-c4nc(-c5ccccc5)nc(-c5ccccc5)n4)c3)c2)cc1 ; c1ccc(-c2cc(-c3ccccc3)cc(-c3ccc(N4c5ccccc5Sc5ccccc54)c(-c4nc(-c5ccccc5)nc(-c5ccccc5)n4)c3)c2)cc1]

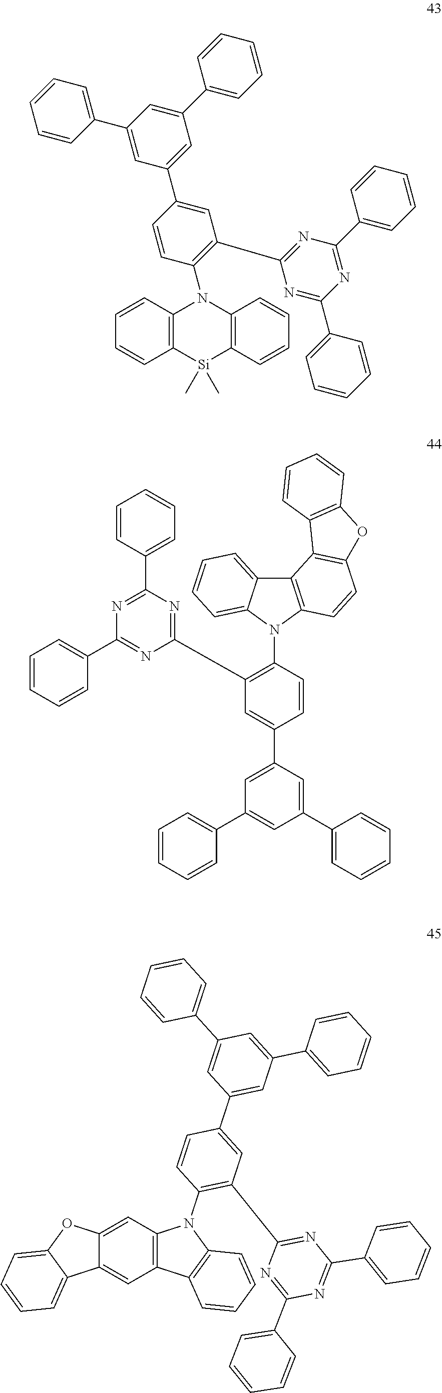 [SMILES: C[Si]1(C)c2ccccc2N(c2ccc(-c3cc(-c4ccccc4)cc(-c4ccccc4)c3)cc2-c2nc(-c3ccccc3)nc(-c3ccccc3)n2)c2ccccc21 ; c1ccc(-c2cc(-c3ccccc3)cc(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6c(cc54)oc4ccccc46)c(-c4nc(-c5ccccc5)nc(-c5ccccc5)n4)c3)c2)cc1]

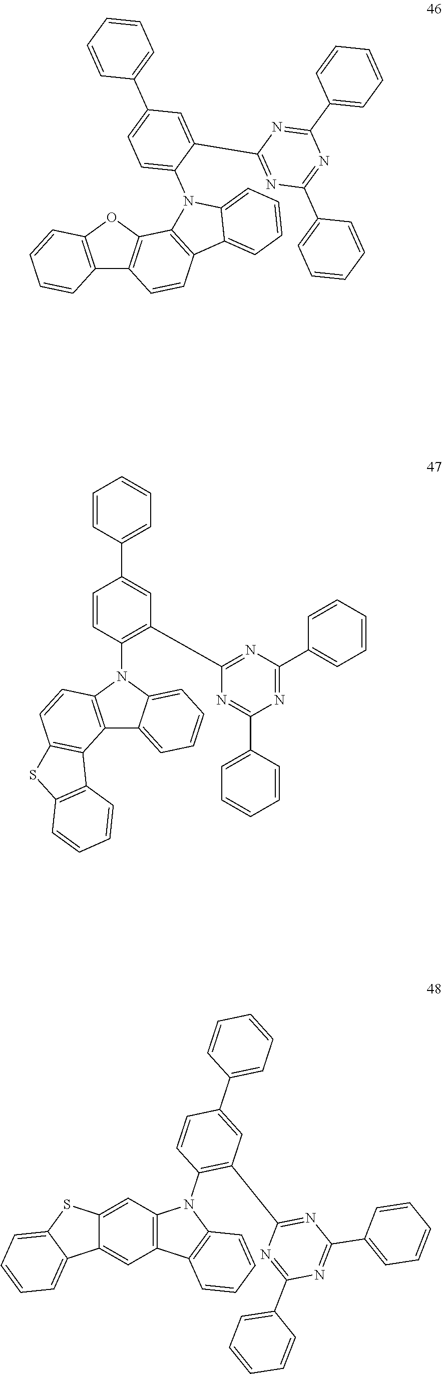 [SMILES: c1ccc(-c2ccc(-n3c4ccccc4c4ccc5c6ccccc6oc5c43)c(-c3nc(-c4ccccc4)nc(-c4ccccc4)n3)c2)cc1 ; c1ccc(-c2ccc(-n3c4ccccc4c4c5c(ccc43)sc3ccccc35)c(-c3nc(-c4ccccc4)nc(-c4ccccc4)n3)c2)cc1 ; c1ccc(-c2ccc(-n3c4ccccc4c4cc5c(cc43)sc3ccccc35)c(-c3nc(-c4ccccc4)nc(-c4ccccc4)n3)c2)cc1]

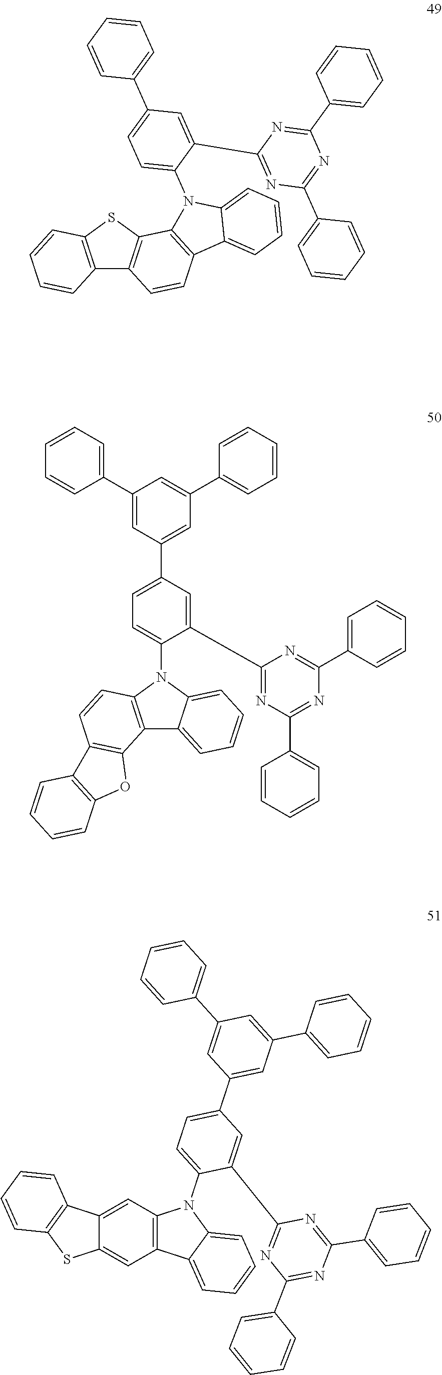 [SMILES: c1ccc(-c2ccc(-n3c4ccccc4c4ccc5c6ccccc6sc5c43)c(-c3nc(-c4ccccc4)nc(-c4ccccc4)n3)c2)cc1 ; c1ccc(-c2cc(-c3ccccc3)cc(-c3ccc(-n4c5ccccc5c5c6oc7ccccc7c6ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6sc7ccccc7c6cc54)c(-c4nc(-c5ccccc5)nc(-c5ccccc5)n4)c3)c2)cc1]

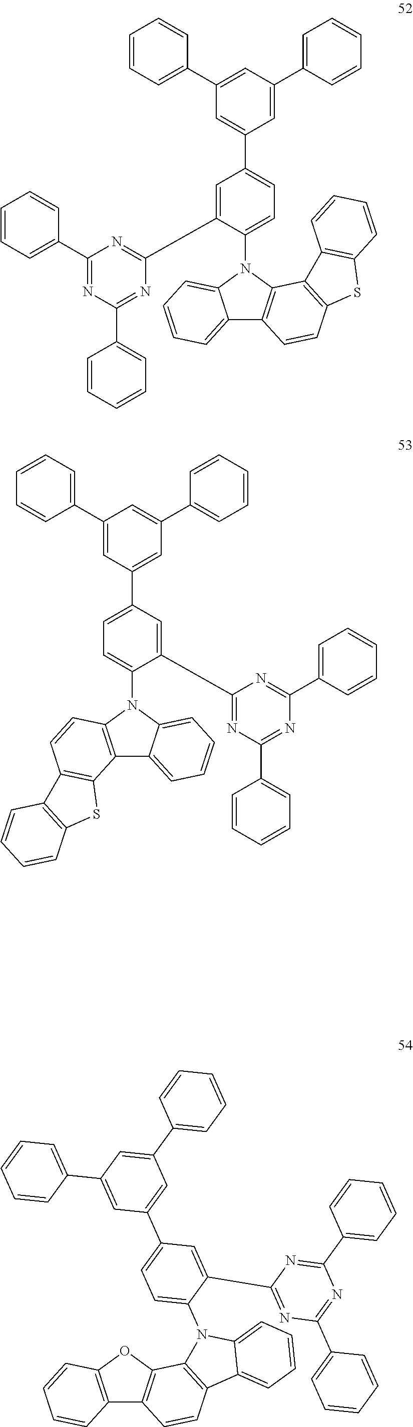 [SMILES: c1ccc(-c2cc(-c3ccccc3)cc(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2cc(-c3ccccc3)cc(-c3ccc(-n4c5ccccc5c5c6sc7ccccc7c6ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5ccc6c7ccccc7oc6c54)c(-c4nc(-c5ccccc5)nc(-c5ccccc5)n4)c3)c2)cc1]

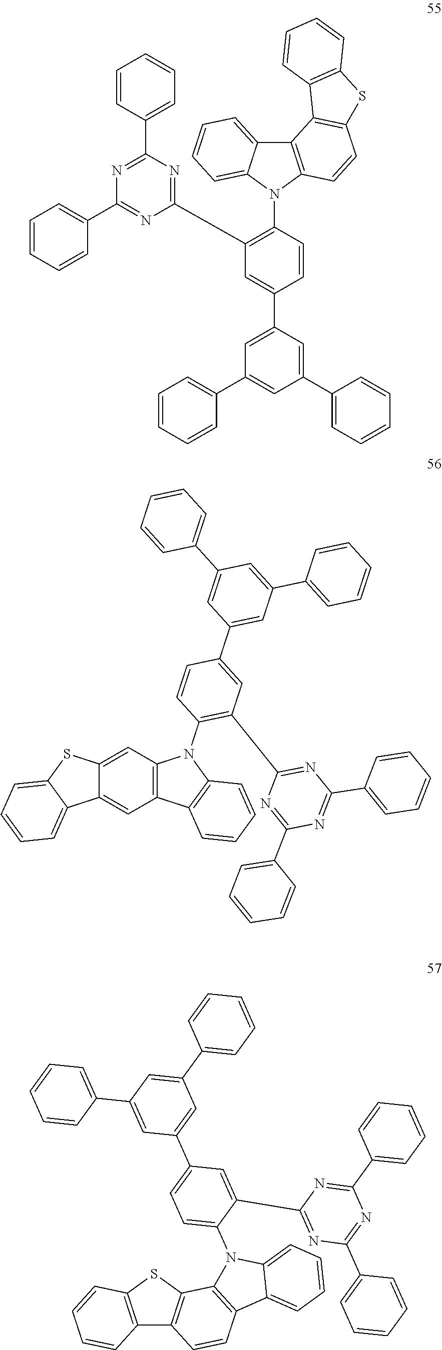

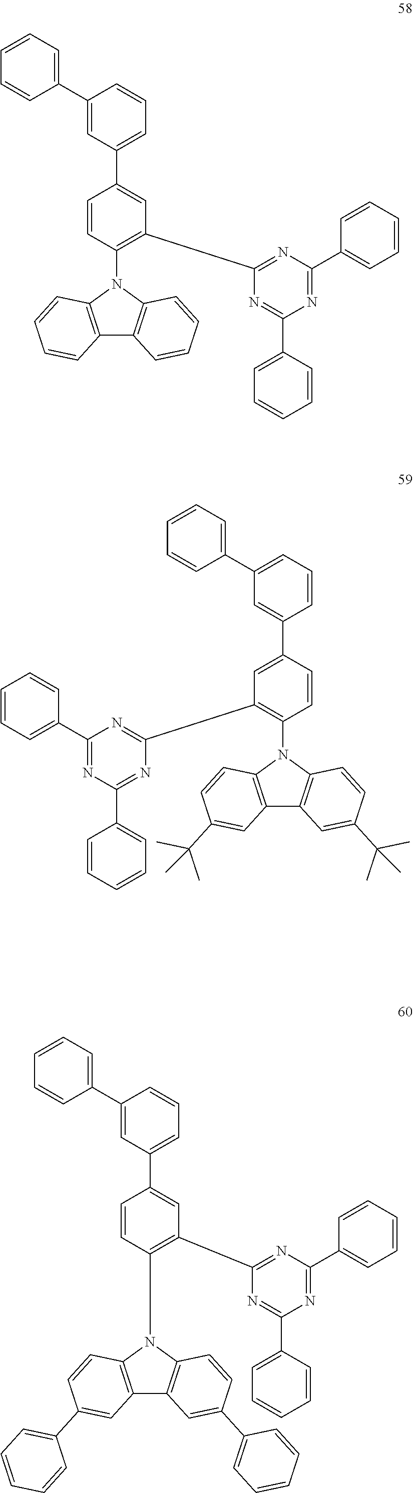 [SMILES: c1ccc(-c2cccc(-c3ccc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ccc(-c2cccc(-c3ccccc3)c2)cc1-c1nc(-c2ccccc2)nc(-c2ccccc2)n1 ; c1ccc(-c2cccc(-c3ccc(-n4c5ccc(-c6ccccc6)cc5c5cc(-c6ccccc6)ccc54)c(-c4nc(-c5ccccc5)nc(-c5ccccc5)n4)c3)c2)cc1]

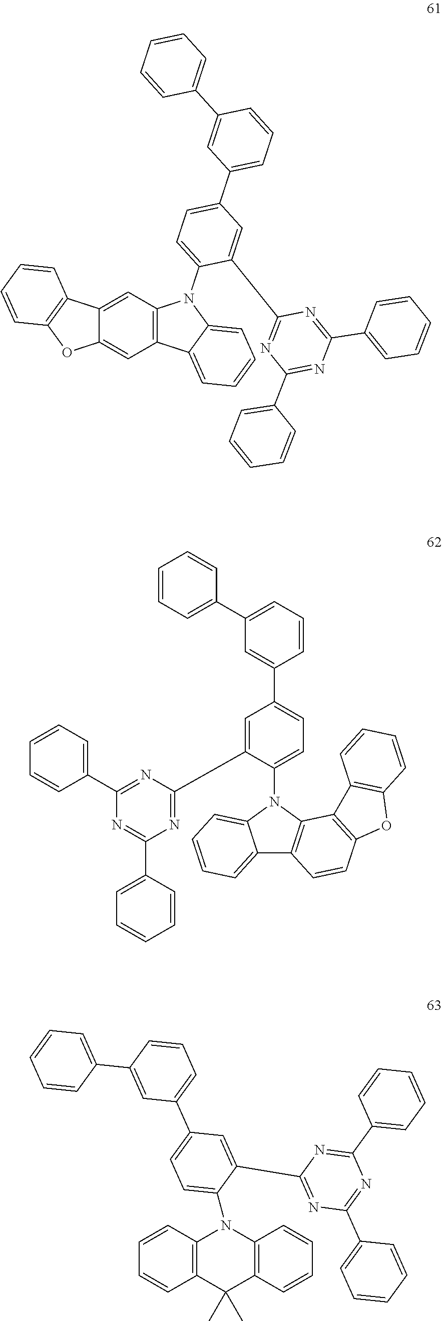 [SMILES: c1ccc(-c2cccc(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2cccc(-c3ccc(-n4c5ccccc5c5ccc6oc7ccccc7c6c54)c(-c4nc(-c5ccccc5)nc(-c5ccccc5)n4)c3)c2)cc1 ; CC1(C)c2ccccc2N(c2ccc(-c3cccc(-c4ccccc4)c3)cc2-c2nc(-c3ccccc3)nc(-c3ccccc3)n2)c2ccccc21]

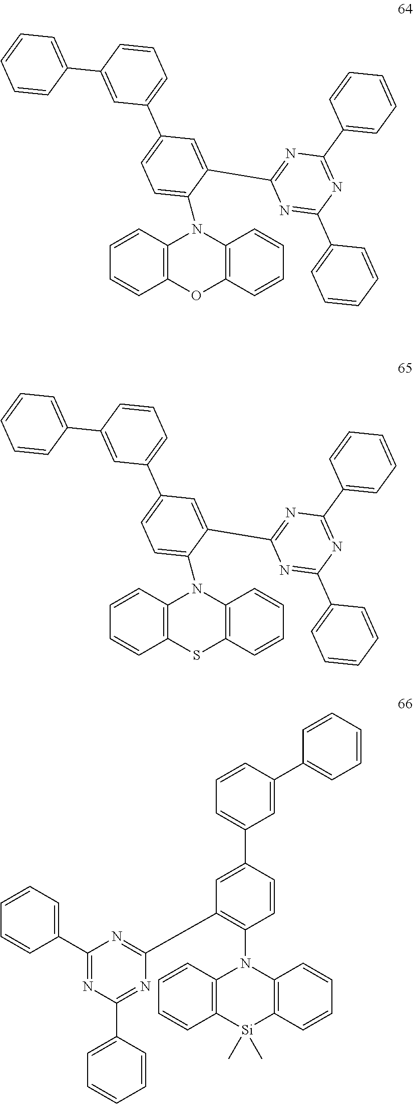 [SMILES: c1ccc(-c2cccc(-c3ccc(N4c5ccccc5Oc5ccccc54)c(-c4nc(-c5ccccc5)nc(-c5ccccc5)n4)c3)c2)cc1 ; c1ccc(-c2cccc(-c3ccc(N4c5ccccc5Sc5ccccc54)c(-c4nc(-c5ccccc5)nc(-c5ccccc5)n4)c3)c2)cc1 ; C[Si]1(C)c2ccccc2N(c2ccc(-c3cccc(-c4ccccc4)c3)cc2-c2nc(-c3ccccc3)nc(-c3ccccc3)n2)c2ccccc21]

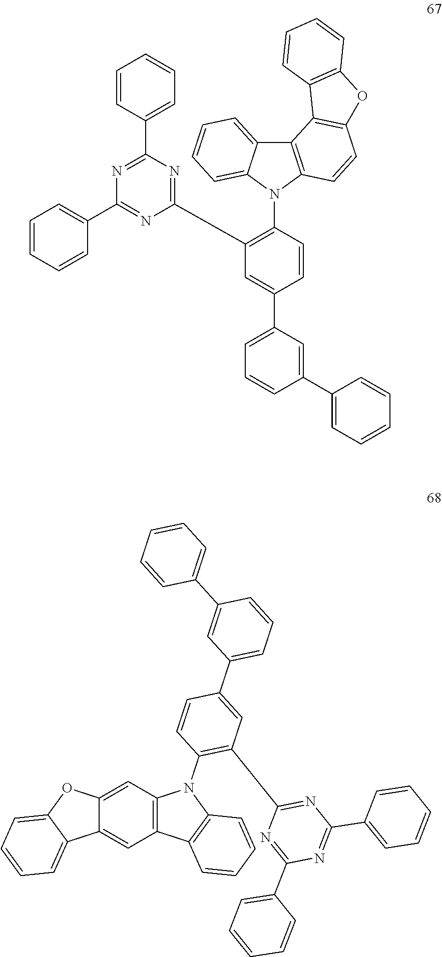 [SMILES: c1ccc(-c2cccc(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2cccc(-c3ccc(-n4c5ccccc5c5cc6c(cc54)oc4ccccc46)c(-c4nc(-c5ccccc5)nc(-c5ccccc5)n4)c3)c2)cc1]

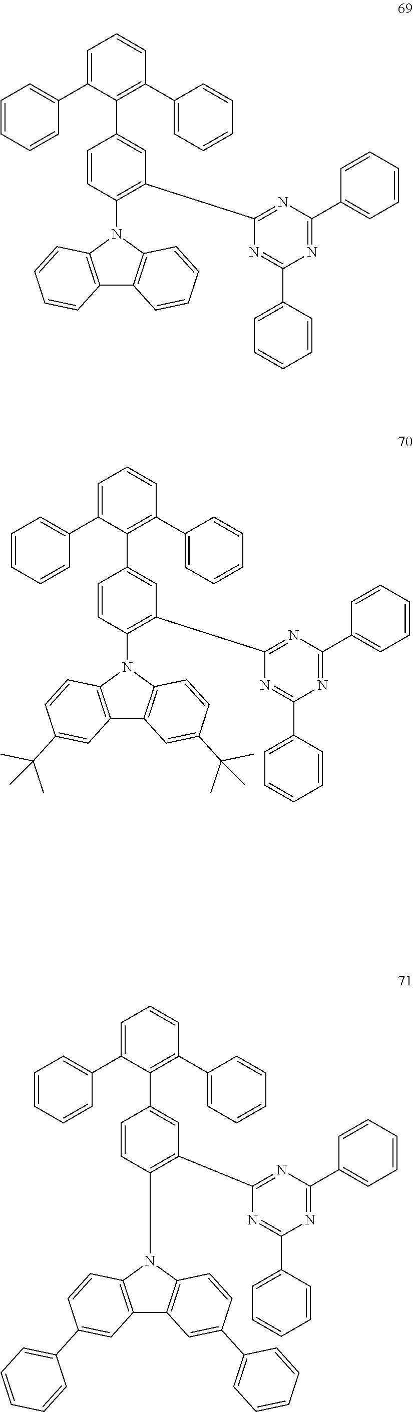 [SMILES: c1ccc(-c2nc(-c3ccccc3)nc(-c3cc(-c4c(-c5ccccc5)cccc4-c4ccccc4)ccc3-n3c4ccccc4c4ccccc43)n2)cc1 ; CC(C)(C)c1ccc2c(c1)c1cc(C(C)(C)C)ccc1n2-c1ccc(-c2c(-c3ccccc3)cccc2-c2ccccc2)cc1-c1nc(-c2ccccc2)nc(-c2ccccc2)n1 ; c1ccc(-c2ccc3c(c2)c2cc(-c4ccccc4)ccc2n3-c2ccc(-c3c(-c4ccccc4)cccc3-c3ccccc3)cc2-c2nc(-c3ccccc3)nc(-c3ccccc3)n2)cc1]

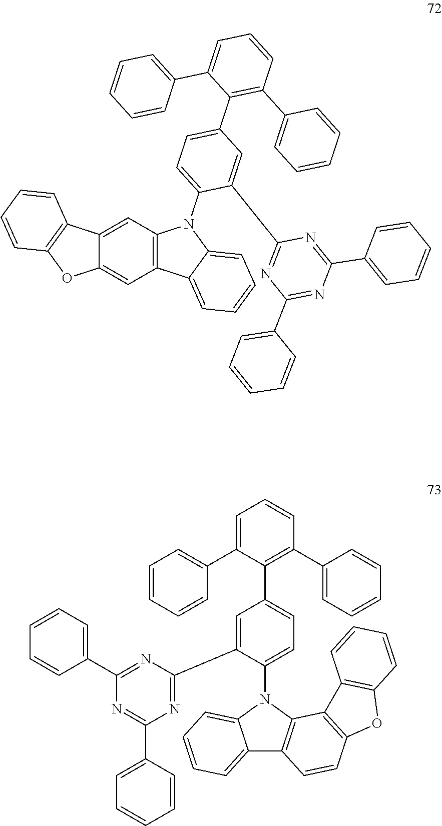 [SMILES: c1ccc(-c2nc(-c3ccccc3)nc(-c3cc(-c4c(-c5ccccc5)cccc4-c4ccccc4)ccc3-n3c4ccccc4c4cc5oc6ccccc6c5cc43)n2)cc1 ; c1ccc(-c2nc(-c3ccccc3)nc(-c3cc(-c4c(-c5ccccc5)cccc4-c4ccccc4)ccc3-n3c4ccccc4c4ccc5oc6ccccc6c5c43)n2)cc1]

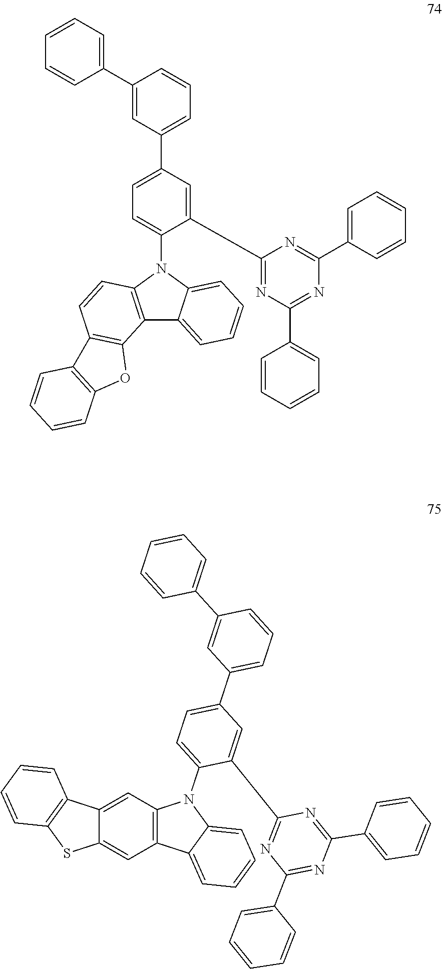 [SMILES: c1ccc(-c2cccc(-c3ccc(-n4c5ccccc5c5c6oc7ccccc7c6ccc54)c(-c4nc(-c5ccccc5)nc(-c5ccccc5)n4)c3)c2)cc1 ; c1ccc(-c2cccc(-c3ccc(-n4c5ccccc5c5cc6sc7ccccc7c6cc54)c(-c4nc(-c5ccccc5)nc(-c5ccccc5)n4)c3)c2)cc1]

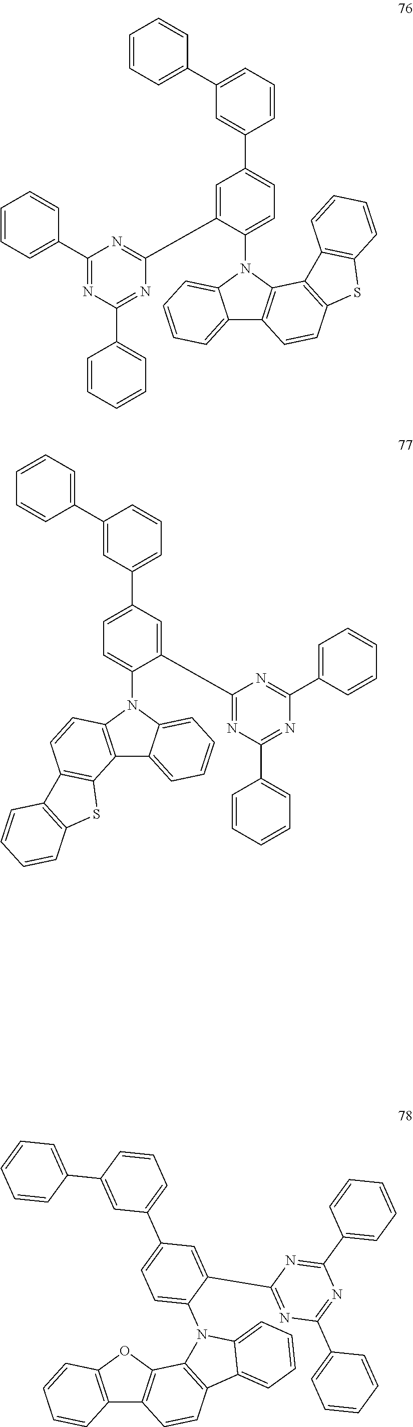 [SMILES: c1ccc(-c2cccc(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2cccc(-c3ccc(-n4c5ccccc5c5c6sc7ccccc7c6ccc54)c(-c4nc(-c5ccccc5)nc(-c5ccccc5)n4)c3)c2)cc1 ; c1ccc(-c2cccc(-c3ccc(-n4c5ccccc5c5ccc6c7ccccc7oc6c54)c(-c4nc(-c5ccccc5)nc(-c5ccccc5)n4)c3)c2)cc1]

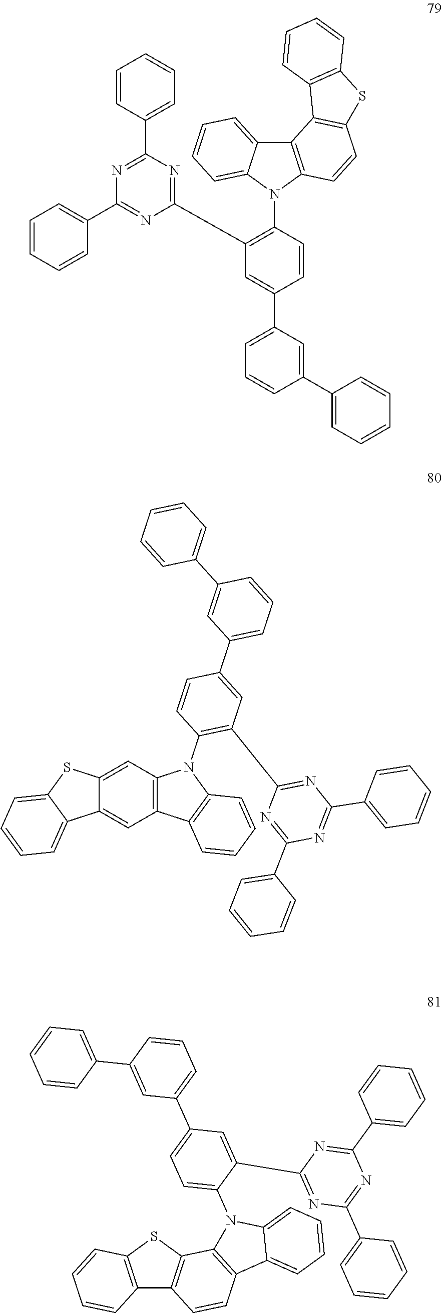 [SMILES: c1ccc(-c2cccc(-c3ccc(-n4c5ccccc5c5c6c(ccc54)sc4ccccc46)c(-c4nc(-c5ccccc5)nc(-c5ccccc5)n4)c3)c2)cc1 ; c1ccc(-c2cccc(-c3ccc(-n4c5ccccc5c5cc6c(cc54)sc4ccccc46)c(-c4nc(-c5ccccc5)nc(-c5ccccc5)n4)c3)c2)cc1 ; c1ccc(-c2cccc(-c3ccc(-n4c5ccccc5c5ccc6c7ccccc7sc6c54)c(-c4nc(-c5ccccc5)nc(-c5ccccc5)n4)c3)c2)cc1]

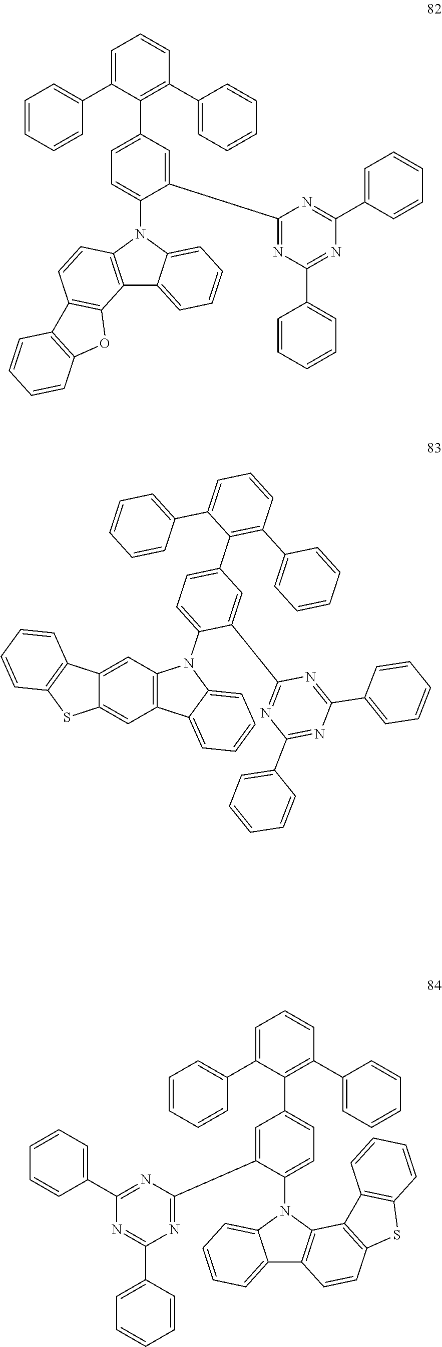 [SMILES: c1ccc(-c2nc(-c3ccccc3)nc(-c3cc(-c4c(-c5ccccc5)cccc4-c4ccccc4)ccc3-n3c4ccccc4c4c5oc6ccccc6c5ccc43)n2)cc1 ; c1ccc(-c2nc(-c3ccccc3)nc(-c3cc(-c4c(-c5ccccc5)cccc4-c4ccccc4)ccc3-n3c4ccccc4c4cc5sc6ccccc6c5cc43)n2)cc1 ; c1ccc(-c2nc(-c3ccccc3)nc(-c3cc(-c4c(-c5ccccc5)cccc4-c4ccccc4)ccc3-n3c4ccccc4c4ccc5sc6ccccc6c5c43)n2)cc1]

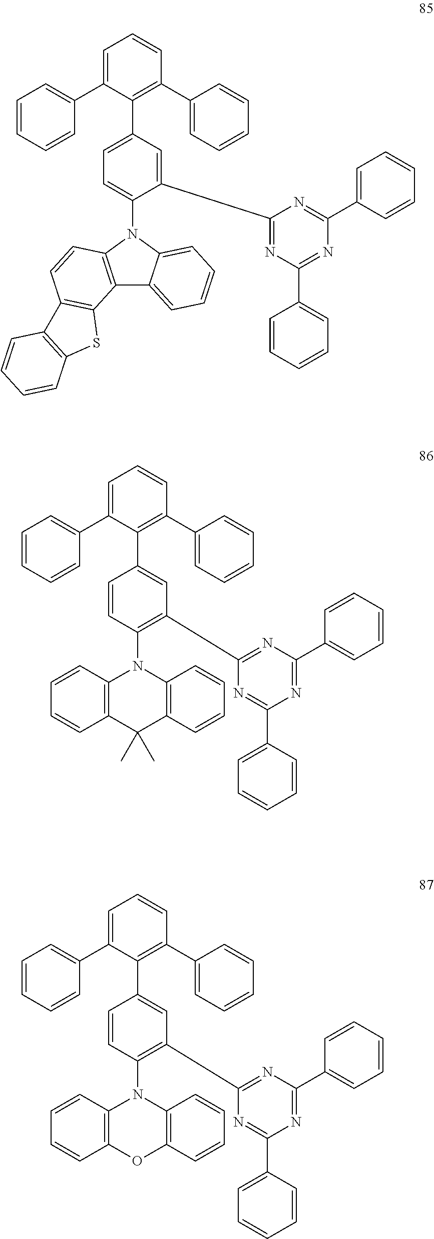 [SMILES: c1ccc(-c2nc(-c3ccccc3)nc(-c3cc(-c4c(-c5ccccc5)cccc4-c4ccccc4)ccc3-n3c4ccccc4c4c5sc6ccccc6c5ccc43)n2)cc1 ; CC1(C)c2ccccc2N(c2ccc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)ccc3N3c4ccccc4Oc4ccccc43)n2)cc1]

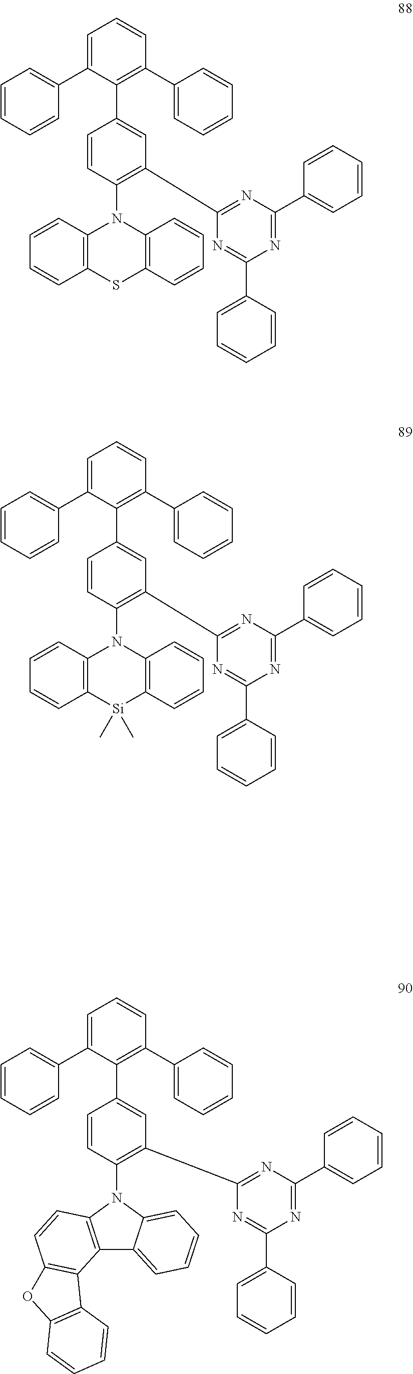 [SMILES: c1ccc(-c2nc(-c3ccccc3)nc(-c3cc(-c4c(-c5ccccc5)cccc4-c4ccccc4)ccc3N3c4ccccc4Sc4ccccc43)n2)cc1 ; C[Si]1(C)c2ccccc2N(c2ccc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)ccc3-n3c4ccccc4c4c5c(ccc43)oc3ccccc35)n2)cc1]

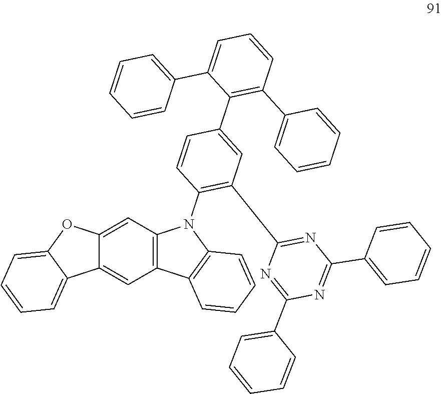 [SMILES: c1ccc(-c2nc(-c3ccccc3)nc(-c3cc(-c4c(-c5ccccc5)cccc4-c4ccccc4)ccc3-n3c4ccccc4c4cc5c(cc43)oc3ccccc35)n2)cc1]

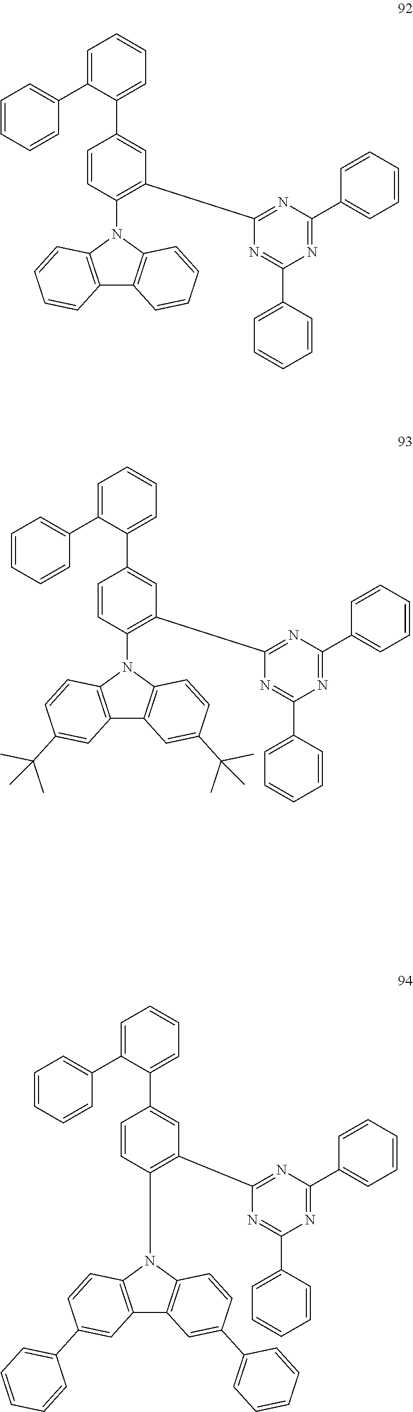 [SMILES: c1ccc(-c2nc(-c3ccccc3)nc(-c3cc(-c4ccccc4-c4ccccc4)ccc3-n3c4ccccc4c4ccccc43)n2)cc1 ; CC(C)(C)c1ccc2c(c1)c1cc(C(C)(C)C)ccc1n2-c1ccc(-c2ccccc2-c2ccccc2)cc1-c1nc(-c2ccccc2)nc(-c2ccccc2)n1 ; c1ccc(-c2ccc3c(c2)c2cc(-c4ccccc4)ccc2n3-c2ccc(-c3ccccc3-c3ccccc3)cc2-c2nc(-c3ccccc3)nc(-c3ccccc3)n2)cc1]

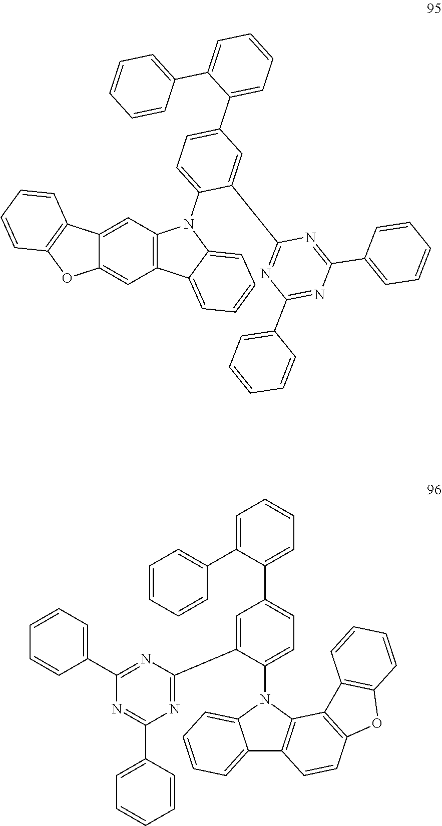 [SMILES: c1ccc(-c2nc(-c3ccccc3)nc(-c3cc(-c4ccccc4-c4ccccc4)ccc3-n3c4ccccc4c4cc5oc6ccccc6c5cc43)n2)cc1 ; c1ccc(-c2nc(-c3ccccc3)nc(-c3cc(-c4ccccc4-c4ccccc4)ccc3-n3c4ccccc4c4ccc5oc6ccccc6c5c43)n2)cc1]

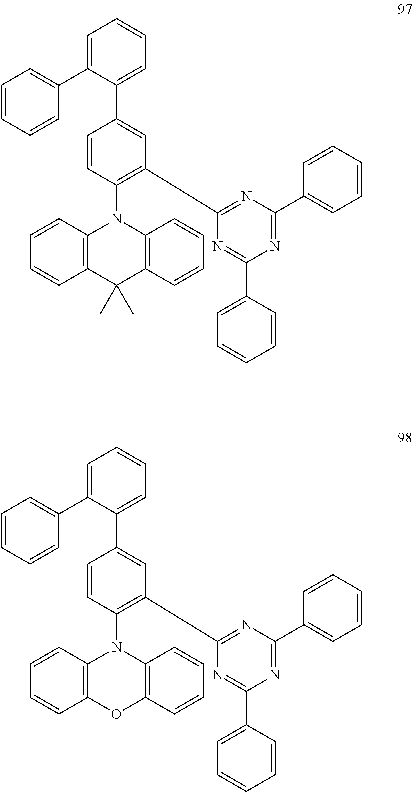 [SMILES: CC1(C)c2ccccc2N(c2ccc(-c3ccccc3-c3ccccc3)cc2-c2nc(-c3ccccc3)nc(-c3ccccc3)n2)c2ccccc21 ; c1ccc(-c2nc(-c3ccccc3)nc(-c3cc(-c4ccccc4-c4ccccc4)ccc3N3c4ccccc4Oc4ccccc43)n2)cc1]

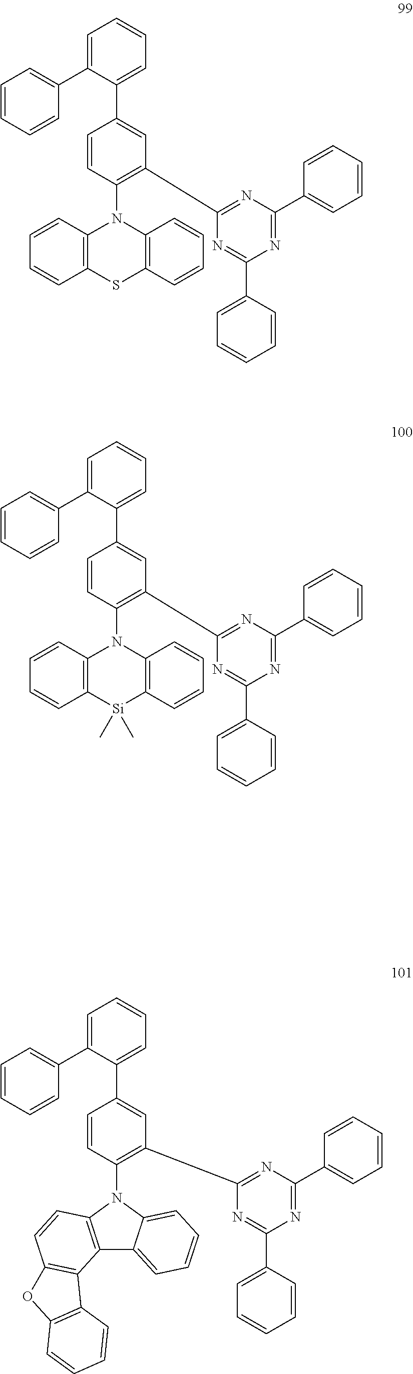 [SMILES: c1ccc(-c2nc(-c3ccccc3)nc(-c3cc(-c4ccccc4-c4ccccc4)ccc3N3c4ccccc4Sc4ccccc43)n2)cc1 ; C[Si]1(C)c2ccccc2N(c2ccc(-c3ccccc3-c3ccccc3)cc2-c2nc(-c3ccccc3)nc(-c3ccccc3)n2)c2ccccc21 ; c1ccc(-c2nc(-c3ccccc3)nc(-c3cc(-c4ccccc4-c4ccccc4)ccc3-n3c4ccccc4c4c5c(ccc43)oc3ccccc35)n2)cc1]

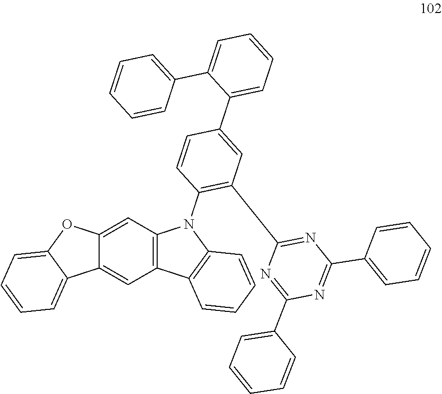 [SMILES: c1ccc(-c2nc(-c3ccccc3)nc(-c3cc(-c4ccccc4-c4ccccc4)ccc3-n3c4ccccc4c4cc5c(cc43)oc3ccccc35)n2)cc1]

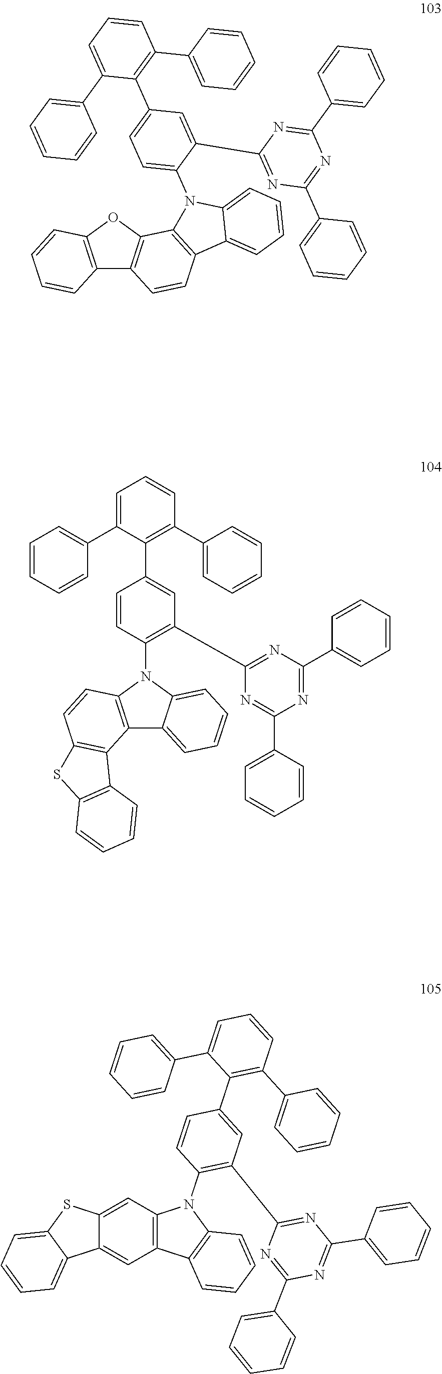 [SMILES: c1ccc(-c2nc(-c3ccccc3)nc(-c3cc(-c4c(-c5ccccc5)cccc4-c4ccccc4)ccc3-n3c4ccccc4c4ccc5c6ccccc6oc5c43)n2)cc1 ; c1ccc(-c2nc(-c3ccccc3)nc(-c3cc(-c4c(-c5ccccc5)cccc4-c4ccccc4)ccc3-n3c4ccccc4c4c5c(ccc43)sc3ccccc35)n2)cc1 ; c1ccc(-c2nc(-c3ccccc3)nc(-c3cc(-c4c(-c5ccccc5)cccc4-c4ccccc4)ccc3-n3c4ccccc4c4cc5c(cc43)sc3ccccc35)n2)cc1]

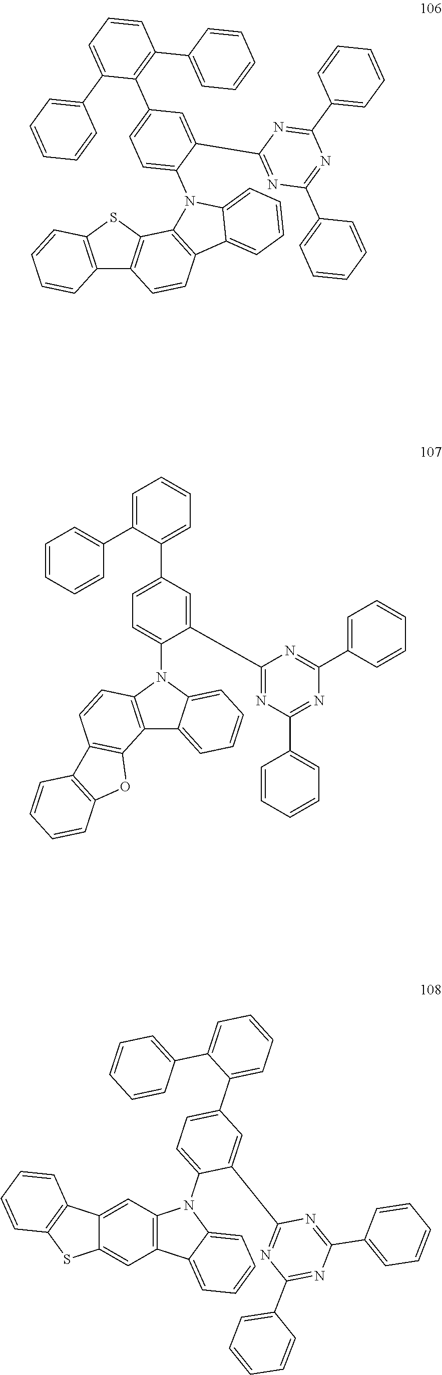 [SMILES: c1ccc(-c2nc(-c3ccccc3)nc(-c3cc(-c4c(-c5ccccc5)cccc4-c4ccccc4)ccc3-n3c4ccccc4c4ccc5c6ccccc6sc5c43)n2)cc1 ; c1ccc(-c2nc(-c3ccccc3)nc(-c3cc(-c4ccccc4-c4ccccc4)ccc3-n3c4ccccc4c4c5oc6ccccc6c5ccc43)n2)cc1 ; c1ccc(-c2nc(-c3ccccc3)nc(-c3cc(-c4ccccc4-c4ccccc4)ccc3-n3c4ccccc4c4cc5sc6ccccc6c5cc43)n2)cc1]

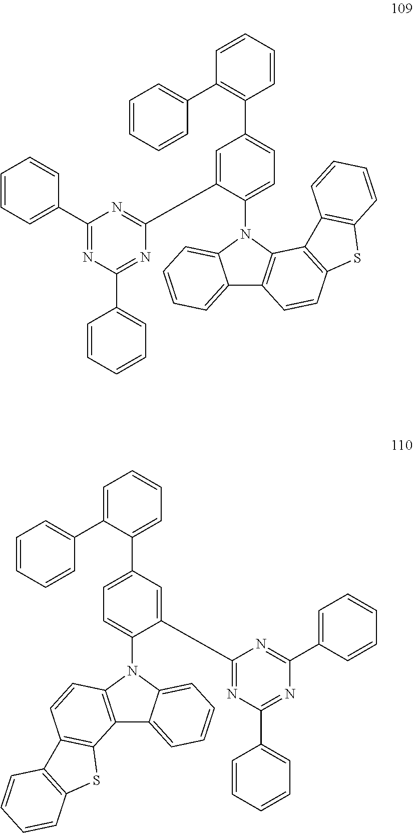 [SMILES: c1ccc(-c2nc(-c3ccccc3)nc(-c3cc(-c4ccccc4-c4ccccc4)ccc3-n3c4ccccc4c4ccc5sc6ccccc6c5c43)n2)cc1 ; c1ccc(-c2nc(-c3ccccc3)nc(-c3cc(-c4ccccc4-c4ccccc4)ccc3-n3c4ccccc4c4c5sc6ccccc6c5ccc43)n2)cc1]

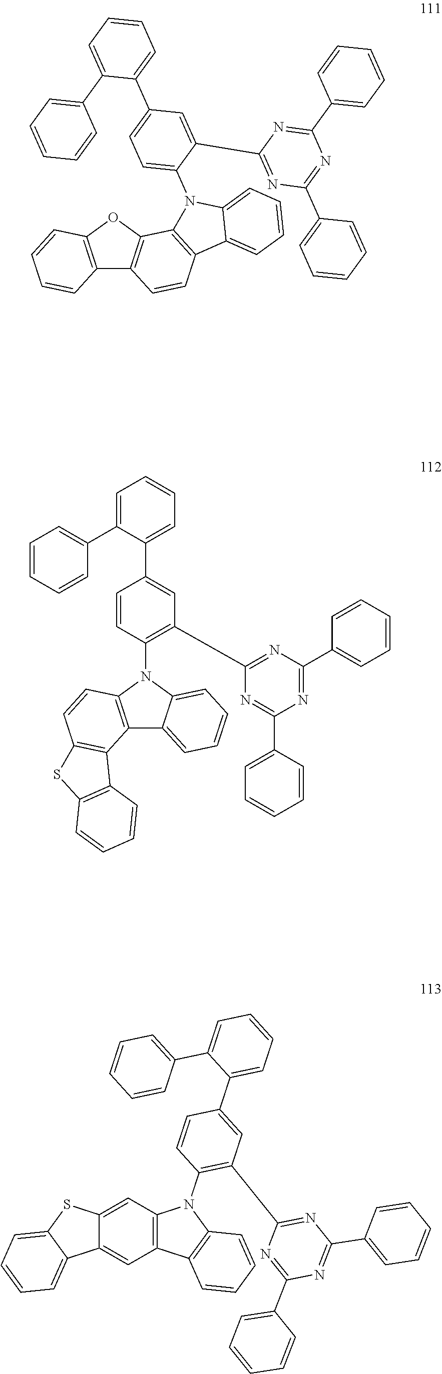 [SMILES: c1ccc(-c2nc(-c3ccccc3)nc(-c3cc(-c4ccccc4-c4ccccc4)ccc3-n3c4ccccc4c4ccc5c6ccccc6oc5c43)n2)cc1 ; c1ccc(-c2nc(-c3ccccc3)nc(-c3cc(-c4ccccc4-c4ccccc4)ccc3-n3c4ccccc4c4c5c(ccc43)sc3ccccc35)n2)cc1 ; c1ccc(-c2nc(-c3ccccc3)nc(-c3cc(-c4ccccc4-c4ccccc4)ccc3-n3c4ccccc4c4cc5c(cc43)sc3ccccc35)n2)cc1]

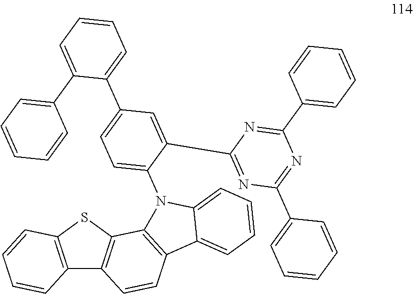 [SMILES: c1ccc(-c2nc(-c3ccccc3)nc(-c3cc(-c4ccccc4-c4ccccc4)ccc3-n3c4ccccc4c4ccc5c6ccccc6sc5c43)n2)cc1]

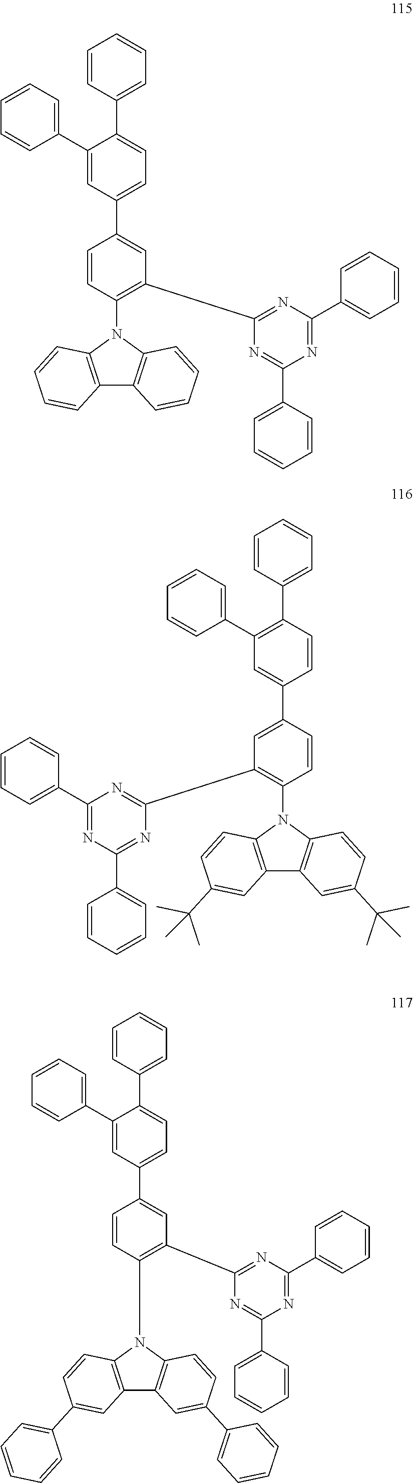 [SMILES: c1ccc(-c2nc(-c3ccccc3)nc(-c3cc(-c4ccc(-c5ccccc5)c(-c5ccccc5)c4)ccc3-n3c4ccccc4c4ccccc43)n2)cc1 ; CC(C)(C)c1ccc2c(c1)c1cc(C(C)(C)C)ccc1n2-c1ccc(-c2ccc(-c3ccccc3)c(-c3ccccc3)c2)cc1-c1nc(-c2ccccc2)nc(-c2ccccc2)n1 ; c1ccc(-c2ccc3c(c2)c2cc(-c4ccccc4)ccc2n3-c2ccc(-c3ccc(-c4ccccc4)c(-c4ccccc4)c3)cc2-c2nc(-c3ccccc3)nc(-c3ccccc3)n2)cc1]

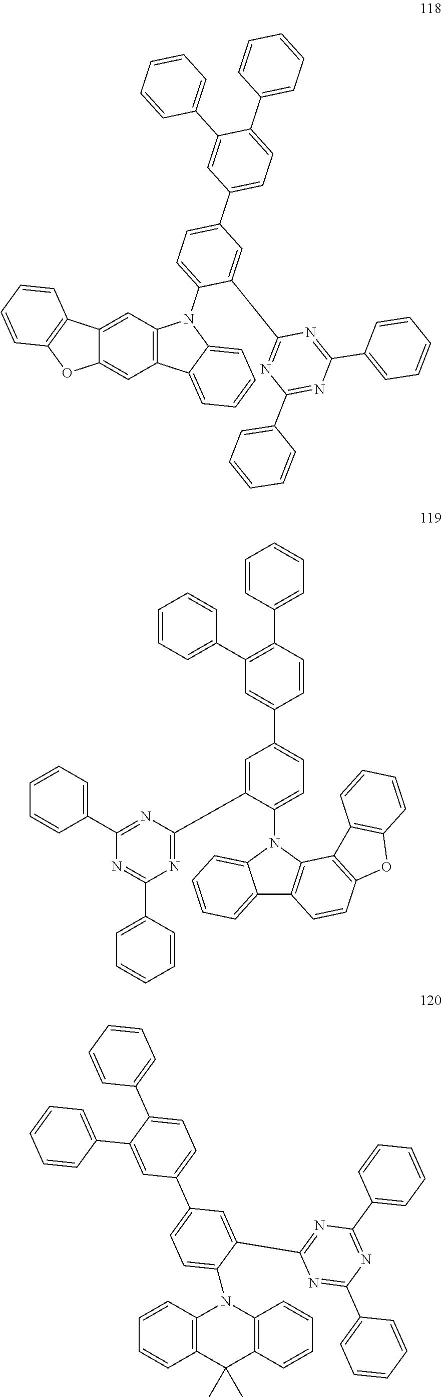

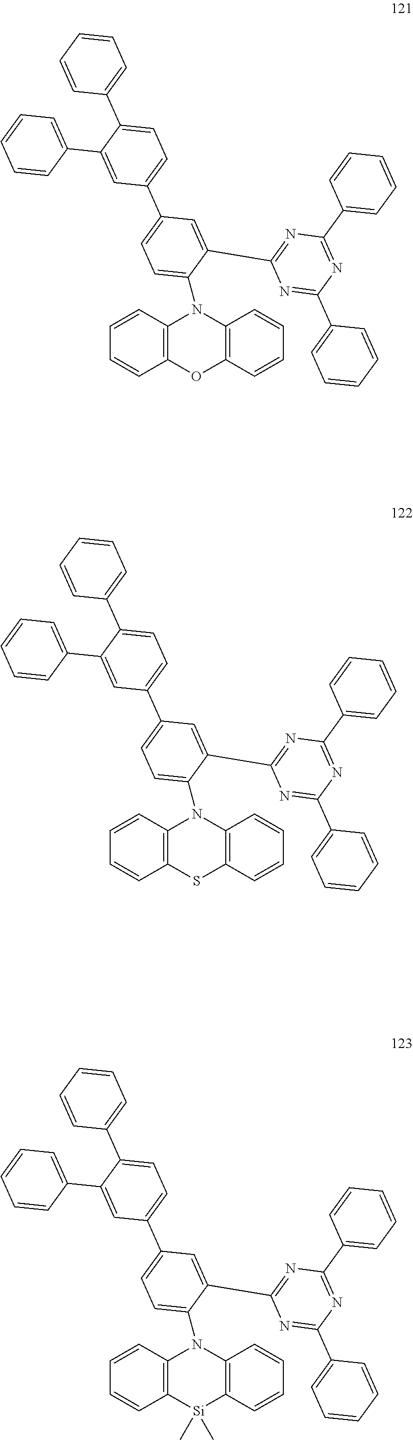 [SMILES: c1ccc(-c2nc(-c3ccccc3)nc(-c3cc(-c4ccc(-c5ccccc5)c(-c5ccccc5)c4)ccc3N3c4ccccc4Oc4ccccc43)n2)cc1 ; c1ccc(-c2nc(-c3ccccc3)nc(-c3cc(-c4ccc(-c5ccccc5)c(-c5ccccc5)c4)ccc3N3c4ccccc4Sc4ccccc43)n2)cc1 ; C[Si]1(C)c2ccccc2N(c2ccc(-c3ccc(-c4ccccc4)c(-c4ccccc4)c3)cc2-c2nc(-c3ccccc3)nc(-c3ccccc3)n2)c2ccccc21]

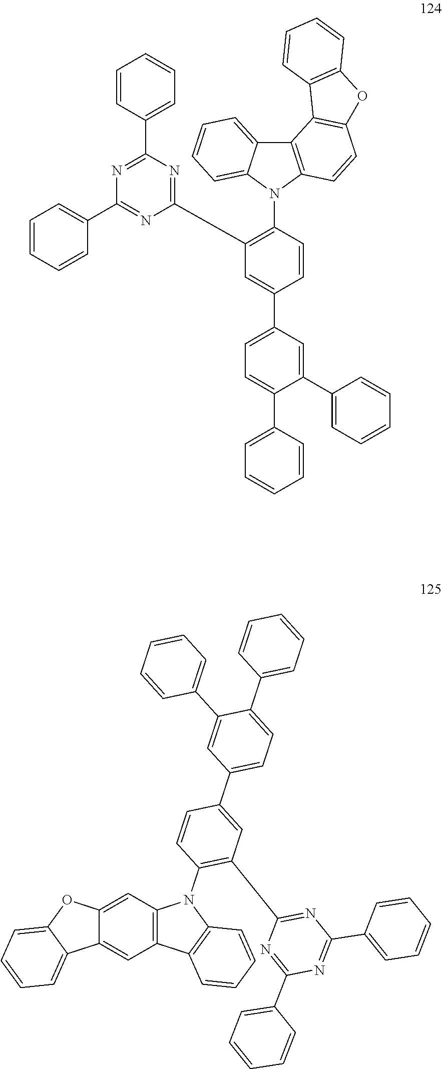 [SMILES: c1ccc(-c2nc(-c3ccccc3)nc(-c3cc(-c4ccc(-c5ccccc5)c(-c5ccccc5)c4)ccc3-n3c4ccccc4c4c5c(ccc43)oc3ccccc35)n2)cc1 ; c1ccc(-c2nc(-c3ccccc3)nc(-c3cc(-c4ccc(-c5ccccc5)c(-c5ccccc5)c4)ccc3-n3c4ccccc4c4cc5c(cc43)oc3ccccc35)n2)cc1]

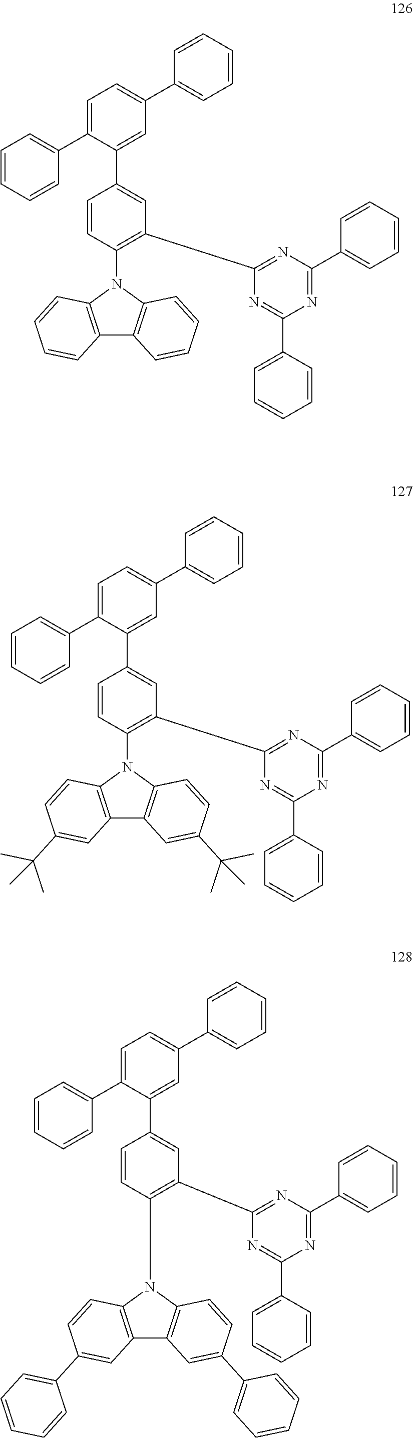 [SMILES: c1ccc(-c2ccc(-c3ccccc3)c(-c3ccc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ccc(-c2cc(-c3ccccc3)ccc2-c2ccccc2)cc1-c1nc(-c2ccccc2)nc(-c2ccccc2)n1 ; c1ccc(-c2ccc(-c3ccccc3)c(-c3ccc(-n4c5ccc(-c6ccccc6)cc5c5cc(-c6ccccc6)ccc54)c(-c4nc(-c5ccccc5)nc(-c5ccccc5)n4)c3)c2)cc1]

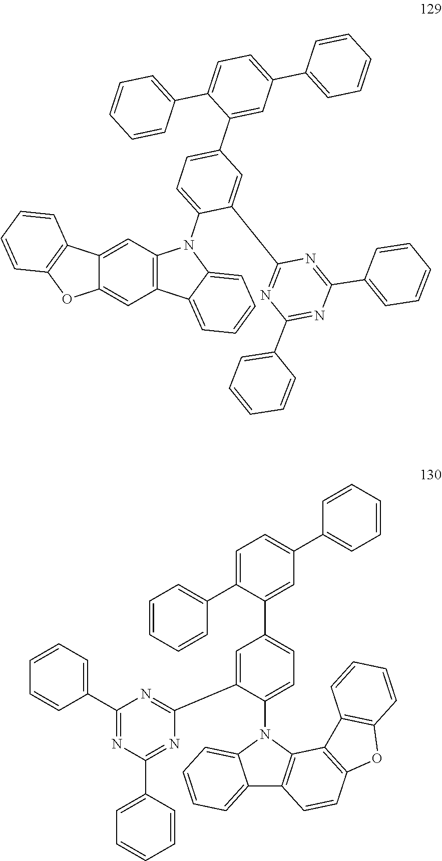 [SMILES: c1ccc(-c2ccc(-c3ccccc3)c(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6oc7ccccc7c6c54)c(-c4nc(-c5ccccc5)nc(-c5ccccc5)n4)c3)c2)cc1]

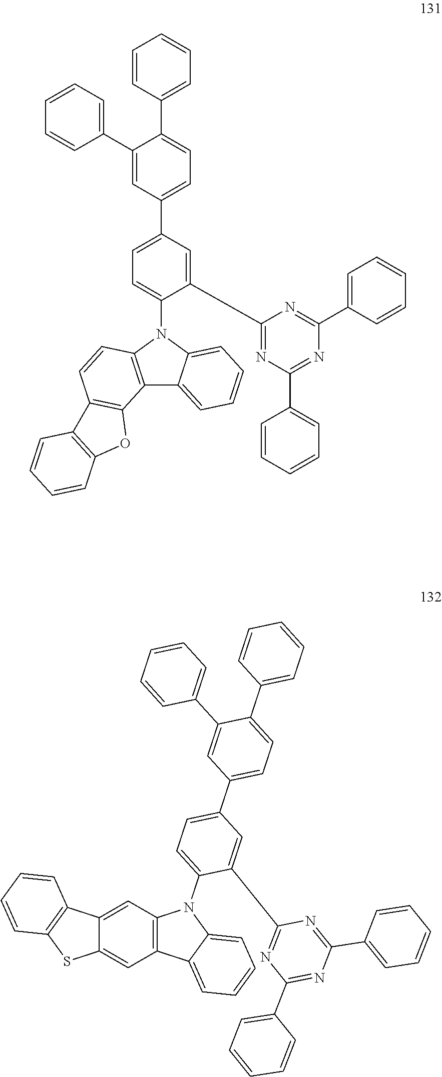 [SMILES: c1ccc(-c2nc(-c3ccccc3)nc(-c3cc(-c4ccc(-c5ccccc5)c(-c5ccccc5)c4)ccc3-n3c4ccccc4c4c5oc6ccccc6c5ccc43)n2)cc1 ; c1ccc(-c2nc(-c3ccccc3)nc(-c3cc(-c4ccc(-c5ccccc5)c(-c5ccccc5)c4)ccc3-n3c4ccccc4c4cc5sc6ccccc6c5cc43)n2)cc1]

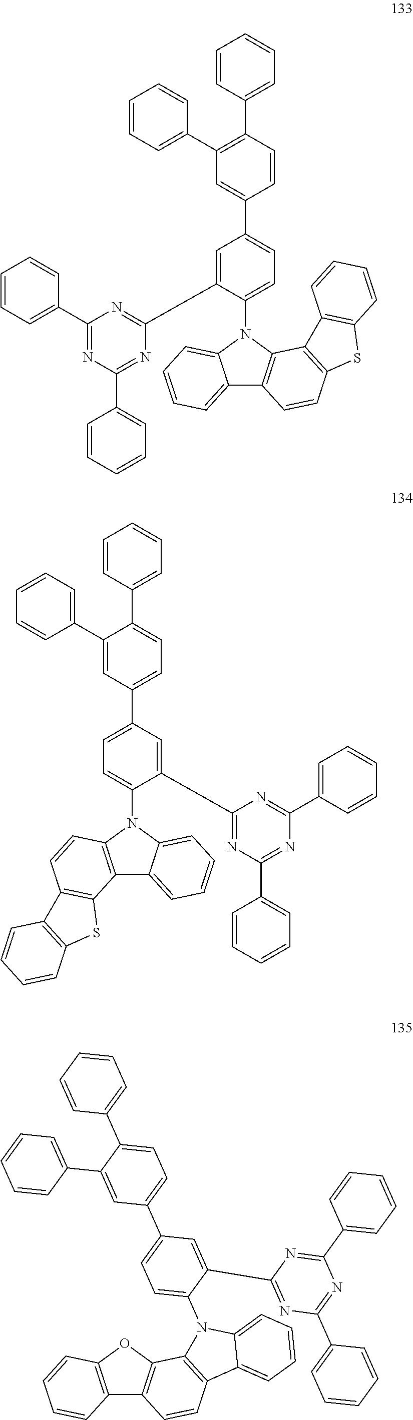 [SMILES: c1ccc(-c2nc(-c3ccccc3)nc(-c3cc(-c4ccc(-c5ccccc5)c(-c5ccccc5)c4)ccc3-n3c4ccccc4c4ccc5sc6ccccc6c5c43)n2)cc1 ; c1ccc(-c2nc(-c3ccccc3)nc(-c3cc(-c4ccc(-c5ccccc5)c(-c5ccccc5)c4)ccc3-n3c4ccccc4c4c5sc6ccccc6c5ccc43)n2)cc1 ; c1ccc(-c2nc(-c3ccccc3)nc(-c3cc(-c4ccc(-c5ccccc5)c(-c5ccccc5)c4)ccc3-n3c4ccccc4c4ccc5c6ccccc6oc5c43)n2)cc1]

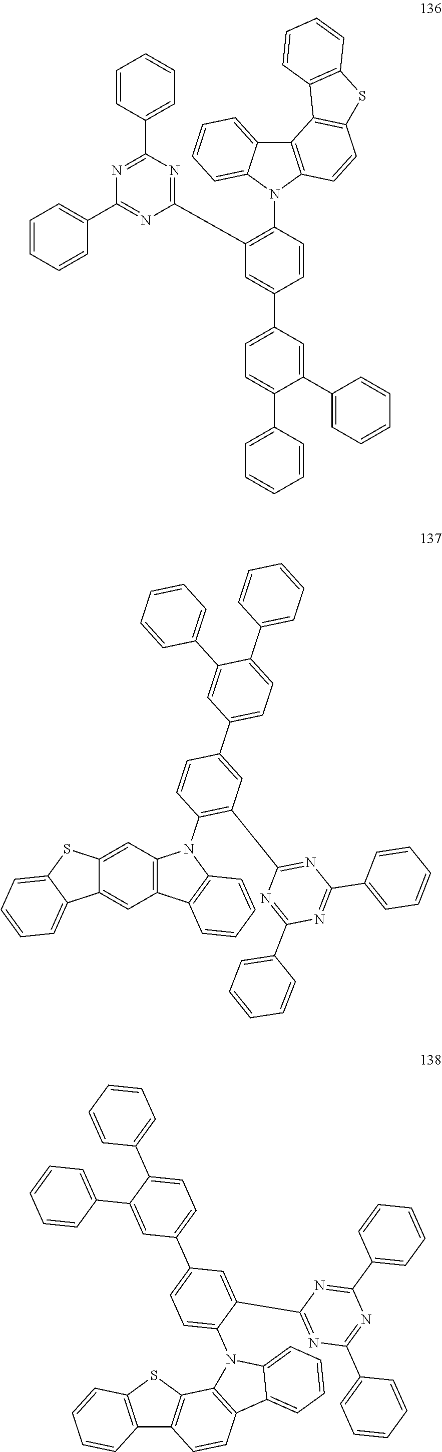 [SMILES: c1ccc(-c2nc(-c3ccccc3)nc(-c3cc(-c4ccc(-c5ccccc5)c(-c5ccccc5)c4)ccc3-n3c4ccccc4c4c5c(ccc43)sc3ccccc35)n2)cc1 ; c1ccc(-c2nc(-c3ccccc3)nc(-c3cc(-c4ccc(-c5ccccc5)c(-c5ccccc5)c4)ccc3-n3c4ccccc4c4cc5c(cc43)sc3ccccc35)n2)cc1 ; c1ccc(-c2nc(-c3ccccc3)nc(-c3cc(-c4ccc(-c5ccccc5)c(-c5ccccc5)c4)ccc3-n3c4ccccc4c4ccc5c6ccccc6sc5c43)n2)cc1]

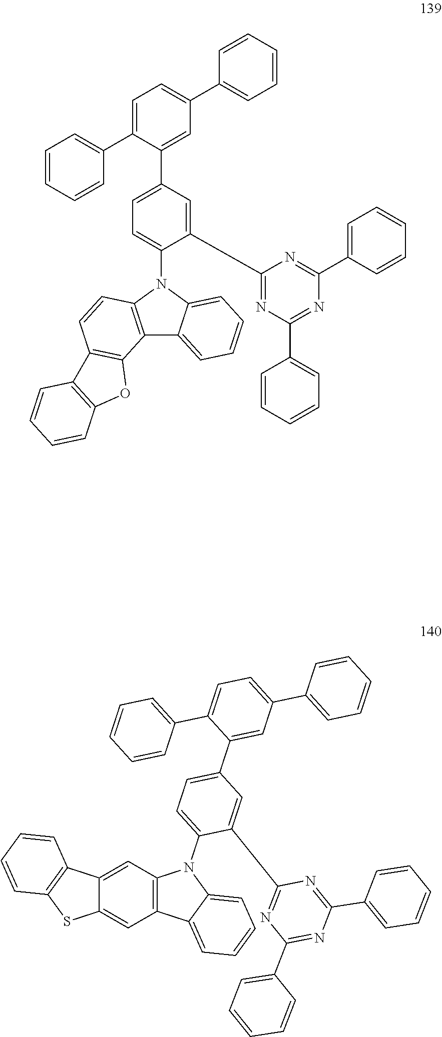 [SMILES: c1ccc(-c2ccc(-c3ccccc3)c(-c3ccc(-n4c5ccccc5c5c6oc7ccccc7c6ccc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5cc6sc7ccccc7c6cc54)c(-c4nc(-c5ccccc5)nc(-c5ccccc5)n4)c3)c2)cc1]

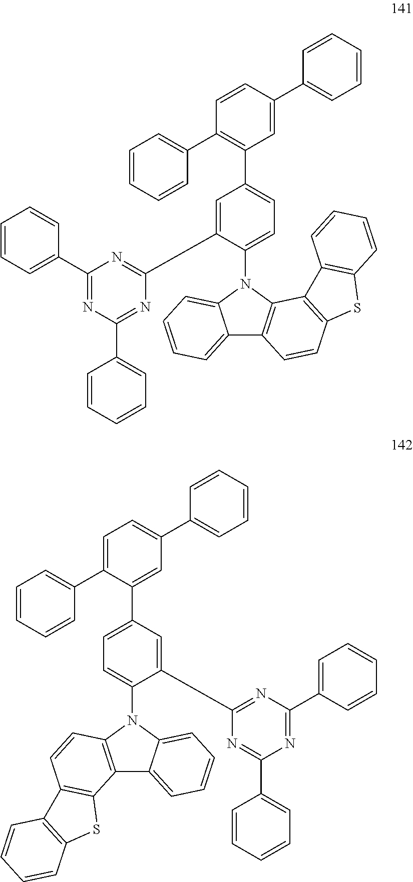 [SMILES: c1ccc(-c2ccc(-c3ccccc3)c(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2ccc(-c3ccccc3)c(-c3ccc(-n4c5ccccc5c5c6sc7ccccc7c6ccc54)c(-c4nc(-c5ccccc5)nc(-c5ccccc5)n4)c3)c2)cc1]

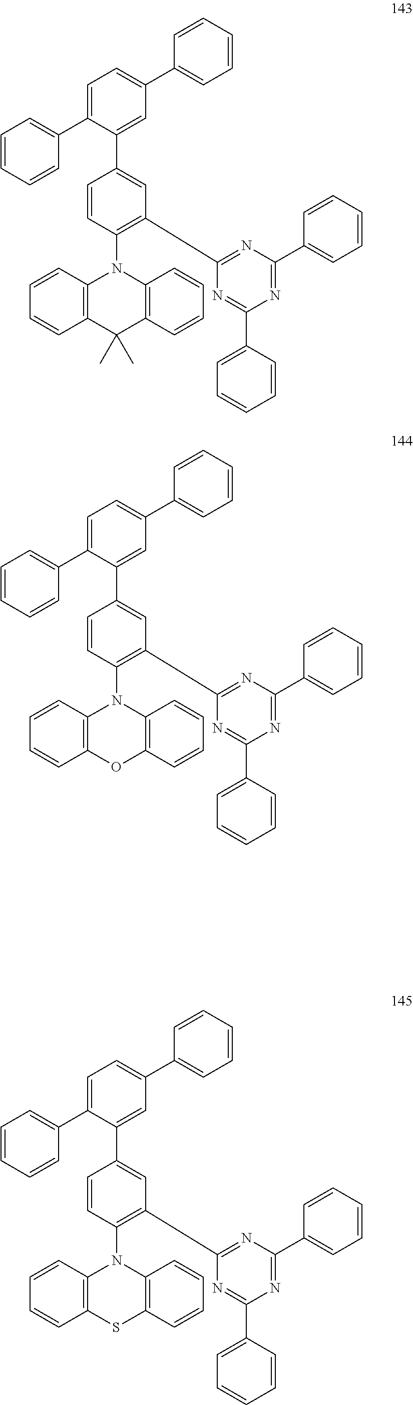 [SMILES: CC1(C)c2ccccc2N(c2ccc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3ccc(N4c5ccccc5Oc5ccccc54)c(-c4nc(-c5ccccc5)nc(-c5ccccc5)n4)c3)c2)cc1 ; c1ccc(-c2ccc(-c3ccccc3)c(-c3ccc(N4c5ccccc5Sc5ccccc54)c(-c4nc(-c5ccccc5)nc(-c5ccccc5)n4)c3)c2)cc1]

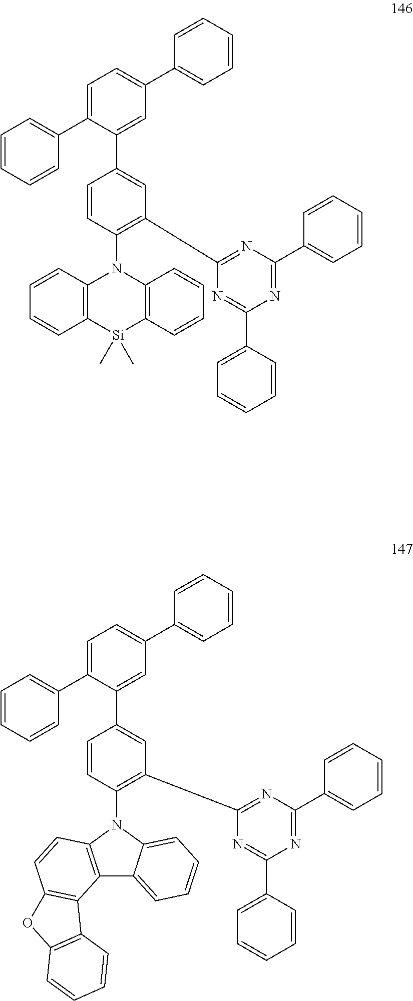 [SMILES: C[Si]1(C)c2ccccc2N(c2ccc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3ccc(-n4c5ccccc5c5c6c(ccc54)oc4ccccc46)c(-c4nc(-c5ccccc5)nc(-c5ccccc5)n4)c3)c2)cc1]

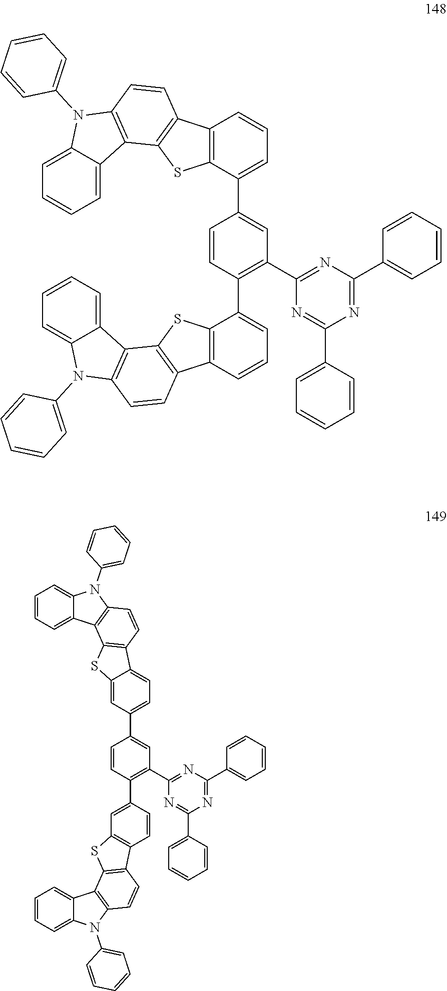 [SMILES: c1ccc(-c2nc(-c3ccccc3)nc(-c3cc(-c4cccc5c4sc4c5ccc5c4c4ccccc4n5-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4ccc5c(c4)sc4c5ccc5c4c4ccccc4n5-c4ccccc4)ccc3-c3ccc4c(c3)sc3c4ccc4c3c3ccccc3n4-c3ccccc3)n2)cc1]

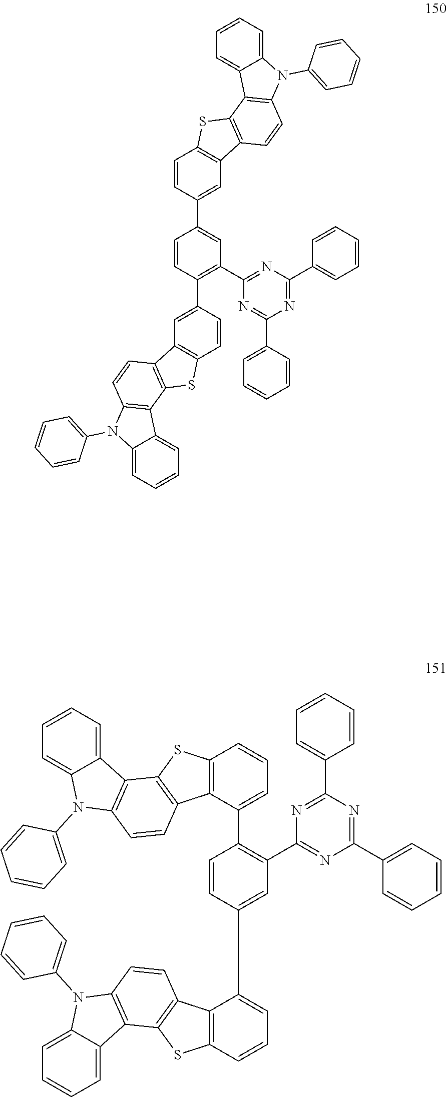 [SMILES: c1ccc(-c2nc(-c3ccccc3)nc(-c3cc(-c4ccc5sc6c(ccc7c6c6ccccc6n7-c6ccccc6)c5c4)ccc3-c3ccc4sc5c(ccc6c5c5ccccc5n6-c5ccccc5)c4c3)n2)cc1 ; c1ccc(-c2nc(-c3ccccc3)nc(-c3cc(-c4cccc5sc6c(ccc7c6c6ccccc6n7-c6ccccc6)c45)ccc3-c3cccc4sc5c(ccc6c5c5ccccc5n6-c5ccccc5)c34)n2)cc1]

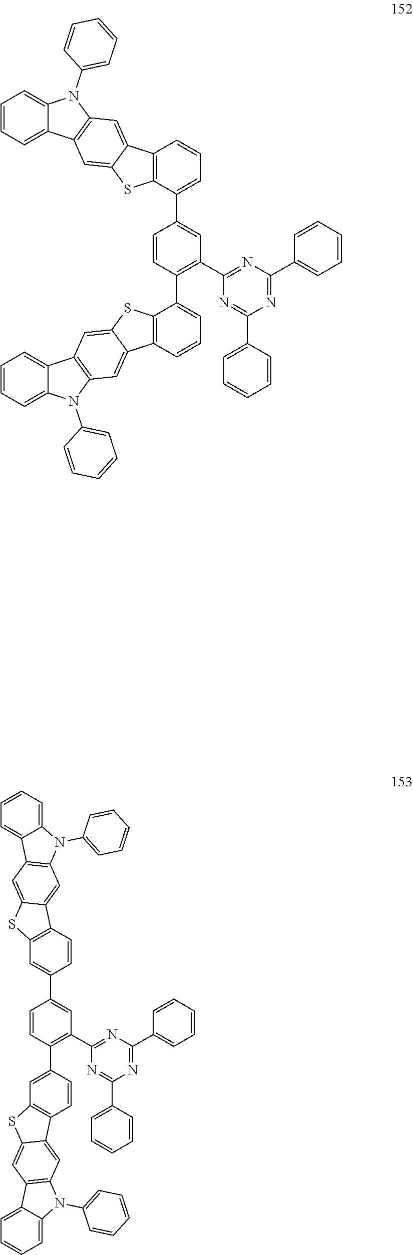 [SMILES: c1ccc(-c2nc(-c3ccccc3)nc(-c3cc(-c4cccc5c4sc4cc6c7ccccc7n(-c7ccccc7)c6cc45)ccc3-c3cccc4c3sc3cc5c6ccccc6n(-c6ccccc6)c5cc34)n2)cc1 ; c1ccc(-c2nc(-c3ccccc3)nc(-c3cc(-c4ccc5c(c4)sc4cc6c7ccccc7n(-c7ccccc7)c6cc45)ccc3-c3ccc4c(c3)sc3cc5c6ccccc6n(-c6ccccc6)c5cc34)n2)cc1]

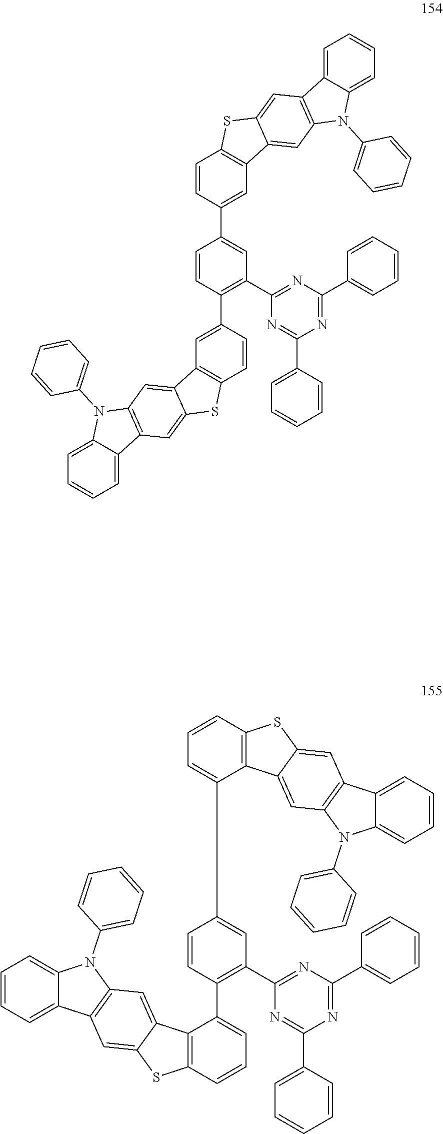 [SMILES: c1ccc(-c2nc(-c3ccccc3)nc(-c3cc(-c4ccc5sc6cc7c8ccccc8n(-c8ccccc8)c7cc6c5c4)ccc3-c3ccc4sc5cc6c7ccccc7n(-c7ccccc7)c6cc5c4c3)n2)cc1 ; c1ccc(-c2nc(-c3ccccc3)nc(-c3cc(-c4cccc5sc6cc7c8ccccc8n(-c8ccccc8)c7cc6c45)ccc3-c3cccc4sc5cc6c7ccccc7n(-c7ccccc7)c6cc5c34)n2)cc1]

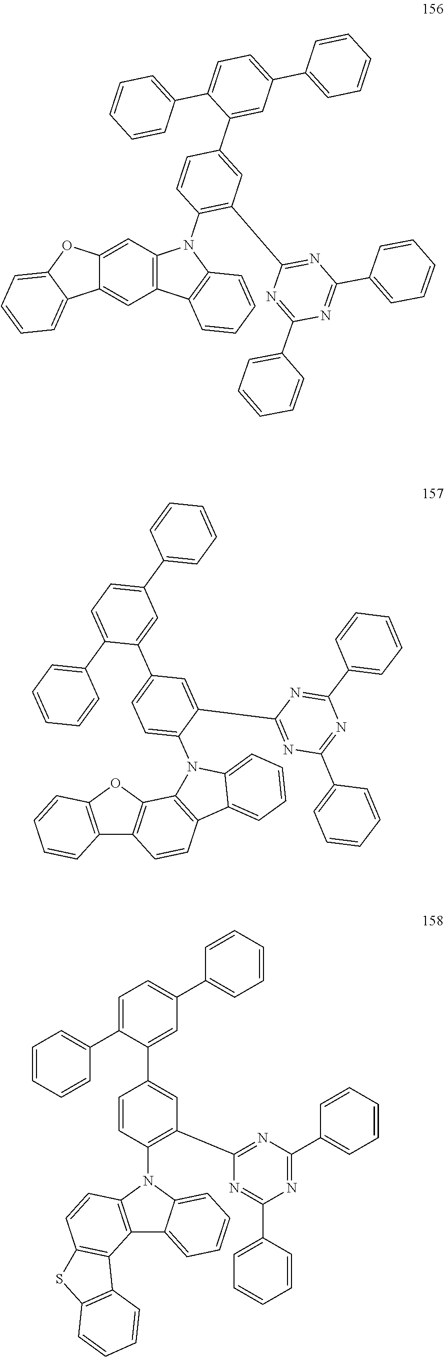 [SMILES: c1ccc(-c2ccc(-c3ccccc3)c(-c3ccc(-n4c5ccccc5c5cc6c(cc54)oc4ccccc46)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6c7ccccc7oc6c54)c(-c4nc(-c5ccccc5)nc(-c5ccccc5)n4)c3)c2)cc1 ; c1ccc(-c2ccc(-c3ccccc3)c(-c3ccc(-n4c5ccccc5c5c6c(ccc54)sc4ccccc46)c(-c4nc(-c5ccccc5)nc(-c5ccccc5)n4)c3)c2)cc1]

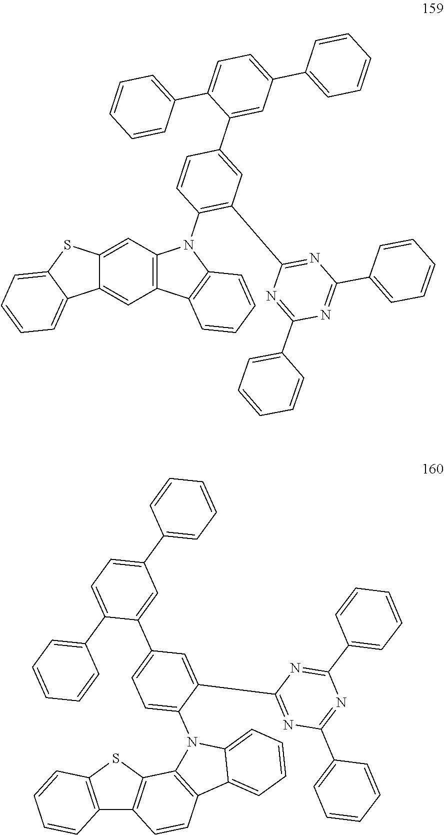 [SMILES: c1ccc(-c2ccc(-c3ccccc3)c(-c3ccc(-n4c5ccccc5c5cc6c(cc54)sc4ccccc46)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6c7ccccc7sc6c54)c(-c4nc(-c5ccccc5)nc(-c5ccccc5)n4)c3)c2)cc1]

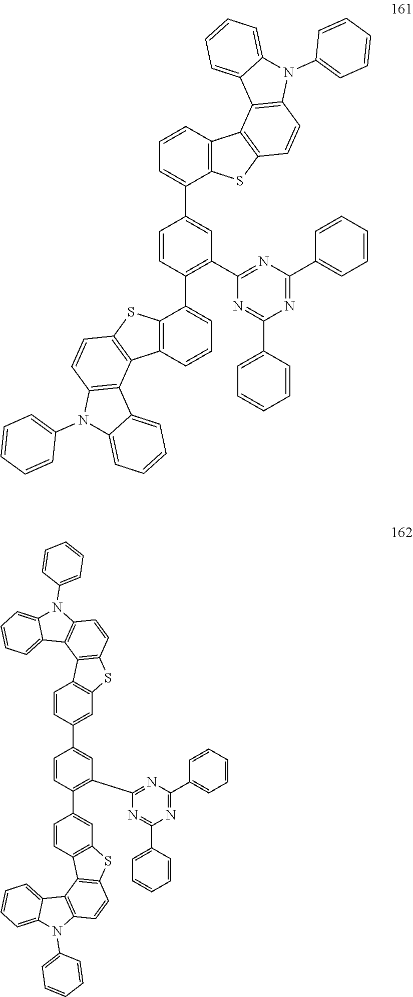 [SMILES: c1ccc(-c2nc(-c3ccccc3)nc(-c3cc(-c4cccc5c4sc4ccc6c(c7ccccc7n6-c6ccccc6)c45)ccc3-c3cccc4c3sc3ccc5c(c6ccccc6n5-c5ccccc5)c34)n2)cc1 ; c1ccc(-c2nc(-c3ccccc3)nc(-c3cc(-c4ccc5c(c4)sc4ccc6c(c7ccccc7n6-c6ccccc6)c45)ccc3-c3ccc4c(c3)sc3ccc5c(c6ccccc6n5-c5ccccc5)c34)n2)cc1]

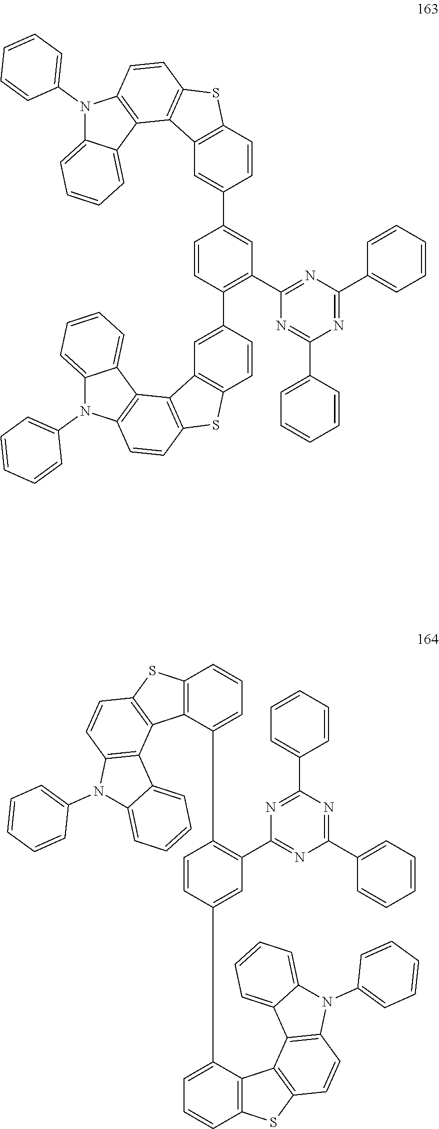 [SMILES: c1ccc(-c2nc(-c3ccccc3)nc(-c3cc(-c4ccc5sc6ccc7c(c8ccccc8n7-c7ccccc7)c6c5c4)ccc3-c3ccc4sc5ccc6c(c7ccccc7n6-c6ccccc6)c5c4c3)n2)cc1 ; c1ccc(-c2nc(-c3ccccc3)nc(-c3cc(-c4cccc5sc6ccc7c(c8ccccc8n7-c7ccccc7)c6c45)ccc3-c3cccc4sc5ccc6c(c7ccccc7n6-c6ccccc6)c5c34)n2)cc1]

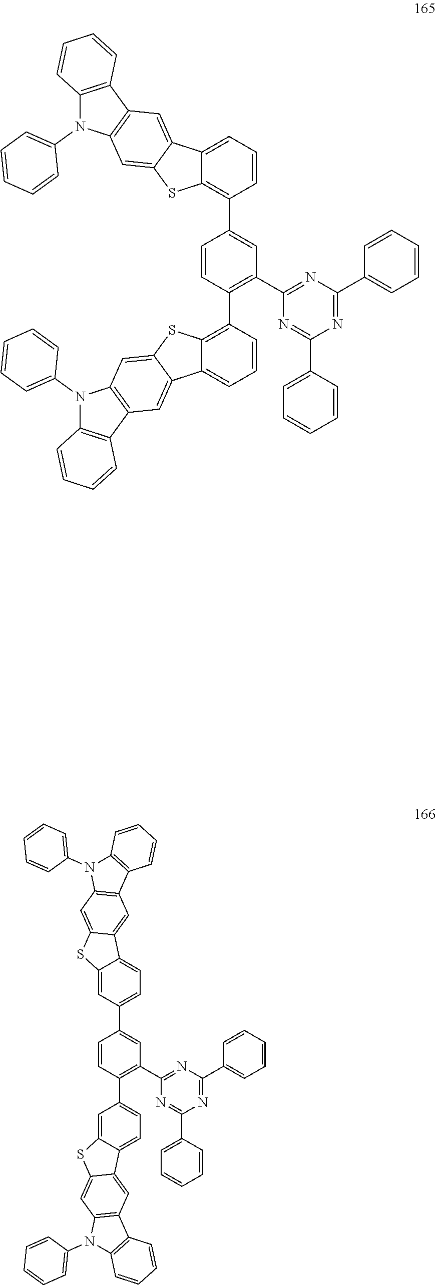 [SMILES: c1ccc(-c2nc(-c3ccccc3)nc(-c3cc(-c4cccc5c4sc4cc6c(cc45)c4ccccc4n6-c4ccccc4)ccc3-c3cccc4c3sc3cc5c(cc34)c3ccccc3n5-c3ccccc3)n2)cc1 ; c1ccc(-c2nc(-c3ccccc3)nc(-c3cc(-c4ccc5c(c4)sc4cc6c(cc45)c4ccccc4n6-c4ccccc4)ccc3-c3ccc4c(c3)sc3cc5c(cc34)c3ccccc3n5-c3ccccc3)n2)cc1]

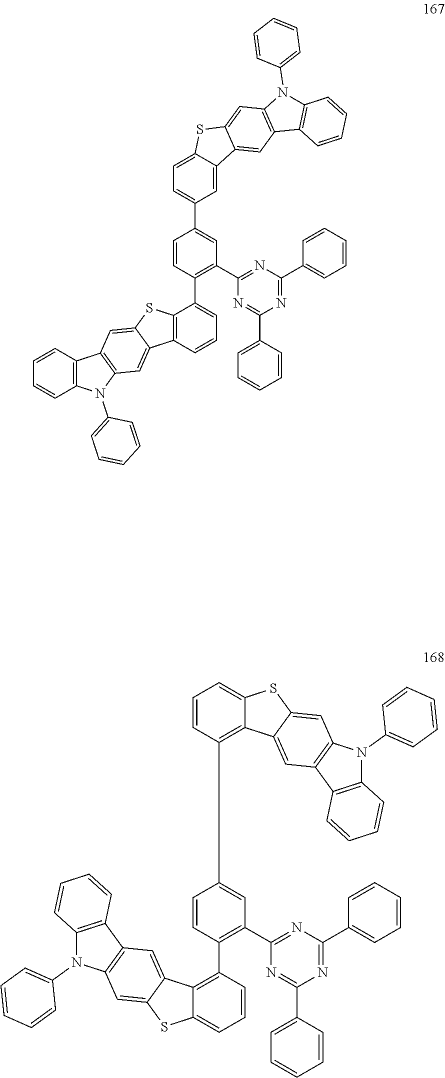 [SMILES: c1ccc(-c2nc(-c3ccccc3)nc(-c3cc(-c4ccc5sc6cc7c(cc6c5c4)c4ccccc4n7-c4ccccc4)ccc3-c3cccc4c3sc3cc5c6ccccc6n(-c6ccccc6)c5cc34)n2)cc1 ; c1ccc(-c2nc(-c3ccccc3)nc(-c3cc(-c4cccc5sc6cc7c(cc6c45)c4ccccc4n7-c4ccccc4)ccc3-c3cccc4sc5cc6c(cc5c34)c3ccccc3n6-c3ccccc3)n2)cc1]

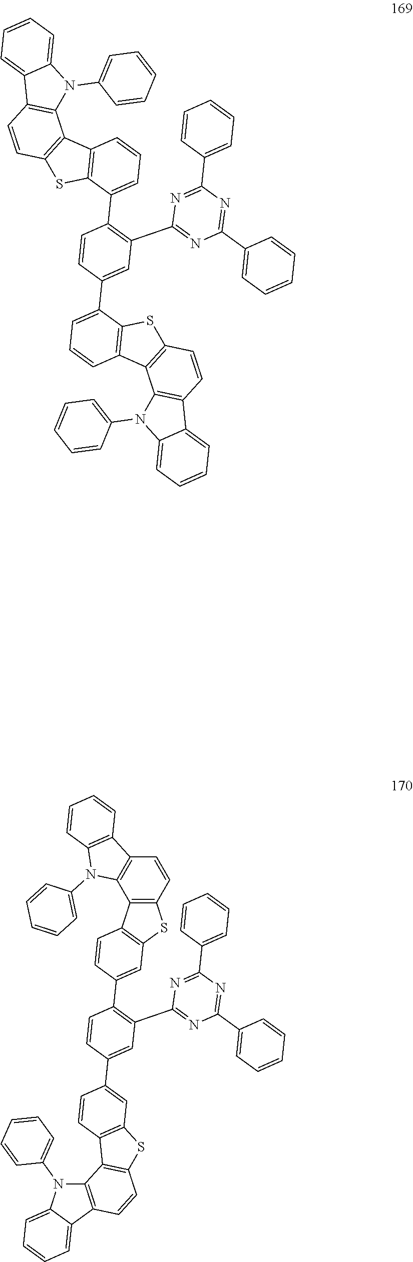 [SMILES: c1ccc(-c2nc(-c3ccccc3)nc(-c3cc(-c4cccc5c4sc4ccc6c7ccccc7n(-c7ccccc7)c6c45)ccc3-c3cccc4c3sc3ccc5c6ccccc6n(-c6ccccc6)c5c34)n2)cc1 ; c1ccc(-c2nc(-c3ccccc3)nc(-c3cc(-c4ccc5c(c4)sc4ccc6c7ccccc7n(-c7ccccc7)c6c45)ccc3-c3ccc4c(c3)sc3ccc5c6ccccc6n(-c6ccccc6)c5c34)n2)cc1]

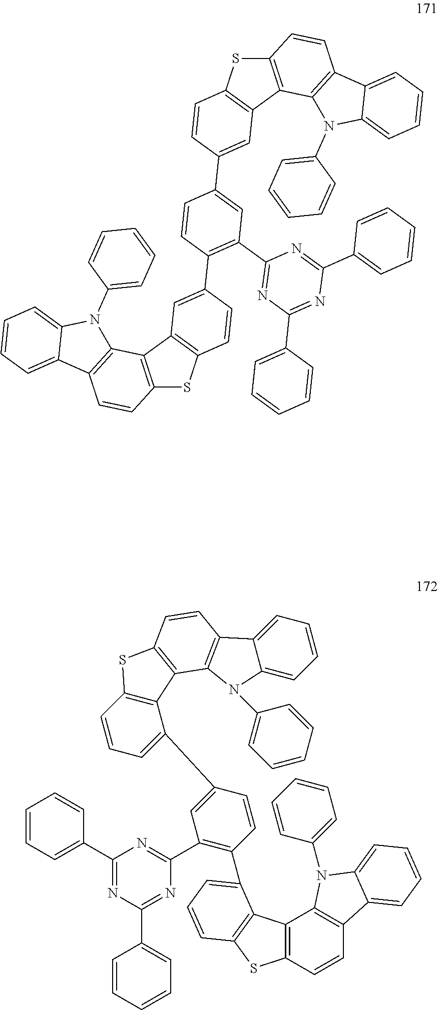 [SMILES: c1ccc(-c2nc(-c3ccccc3)nc(-c3cc(-c4ccc5sc6ccc7c8ccccc8n(-c8ccccc8)c7c6c5c4)ccc3-c3ccc4sc5ccc6c7ccccc7n(-c7ccccc7)c6c5c4c3)n2)cc1 ; c1ccc(-c2nc(-c3ccccc3)nc(-c3cc(-c4cccc5sc6ccc7c8ccccc8n(-c8ccccc8)c7c6c45)ccc3-c3cccc4sc5ccc6c7ccccc7n(-c7ccccc7)c6c5c34)n2)cc1]

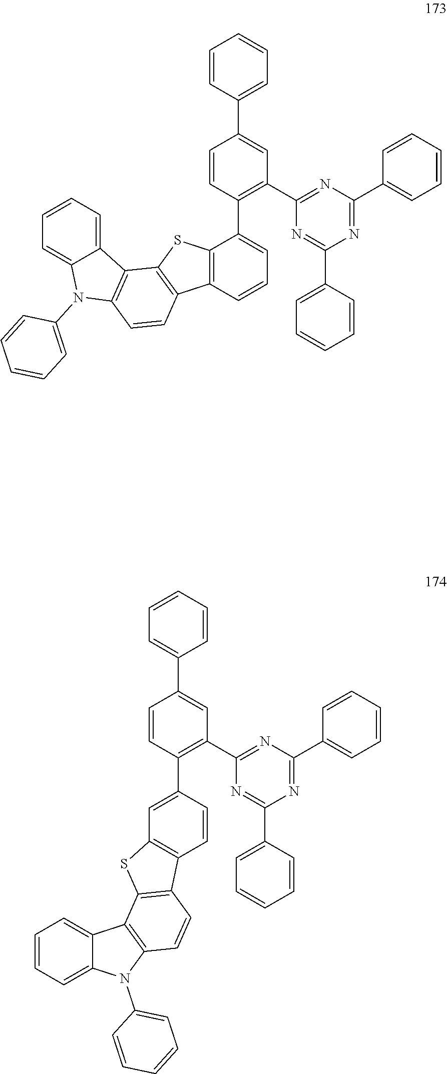 [SMILES: c1ccc(-c2ccc(-c3cccc4c3sc3c4ccc4c3c3ccccc3n4-c3ccccc3)c(-c3nc(-c4ccccc4)nc(-c4ccccc4)n3)c2)cc1 ; c1ccc(-c2ccc(-c3ccc4c(c3)sc3c4ccc4c3c3ccccc3n4-c3ccccc3)c(-c3nc(-c4ccccc4)nc(-c4ccccc4)n3)c2)cc1]

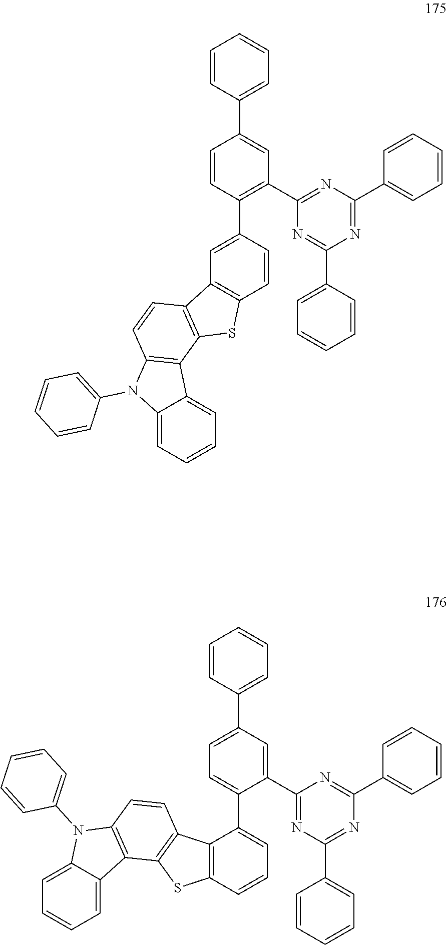 [SMILES: c1ccc(-c2ccc(-c3ccc4sc5c(ccc6c5c5ccccc5n6-c5ccccc5)c4c3)c(-c3nc(-c4ccccc4)nc(-c4ccccc4)n3)c2)cc1 ; c1ccc(-c2ccc(-c3cccc4sc5c(ccc6c5c5ccccc5n6-c5ccccc5)c34)c(-c3nc(-c4ccccc4)nc(-c4ccccc4)n3)c2)cc1]

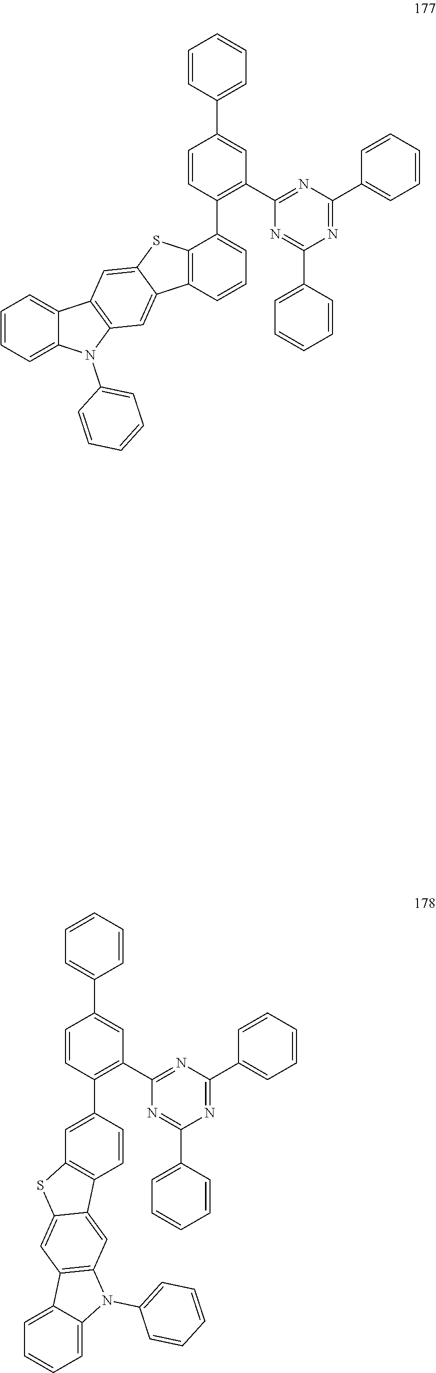 [SMILES: c1ccc(-c2ccc(-c3cccc4c3sc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4c(c3)sc3cc5c6ccccc6n(-c6ccccc6)c5cc34)c(-c3nc(-c4ccccc4)nc(-c4ccccc4)n3)c2)cc1]

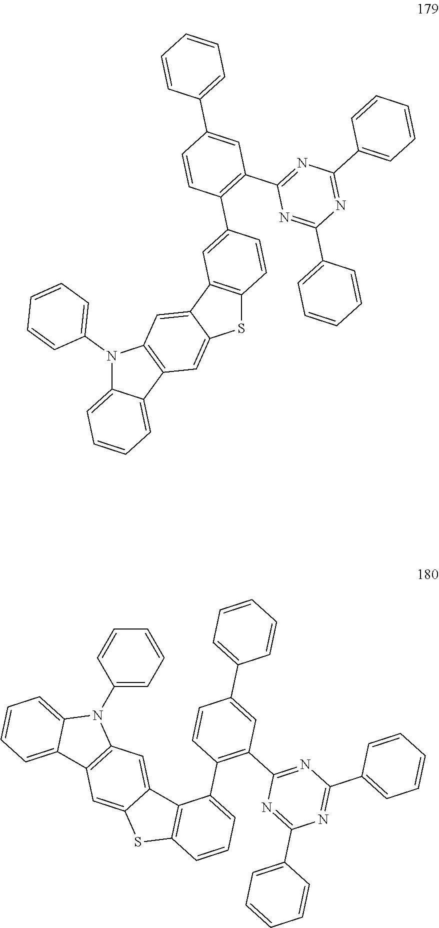 [SMILES: c1ccc(-c2ccc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2ccc(-c3cccc4sc5cc6c7ccccc7n(-c7ccccc7)c6cc5c34)c(-c3nc(-c4ccccc4)nc(-c4ccccc4)n3)c2)cc1]

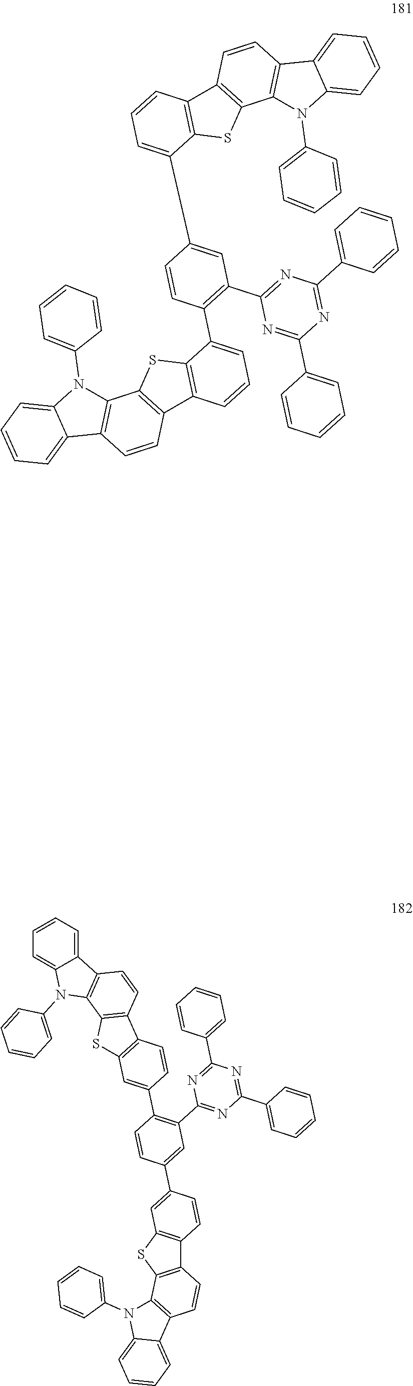 [SMILES: c1ccc(-c2nc(-c3ccccc3)nc(-c3cc(-c4cccc5c4sc4c5ccc5c6ccccc6n(-c6ccccc6)c54)ccc3-c3cccc4c3sc3c4ccc4c5ccccc5n(-c5ccccc5)c43)n2)cc1 ; c1ccc(-c2nc(-c3ccccc3)nc(-c3cc(-c4ccc5c(c4)sc4c5ccc5c6ccccc6n(-c6ccccc6)c54)ccc3-c3ccc4c(c3)sc3c4ccc4c5ccccc5n(-c5ccccc5)c43)n2)cc1]

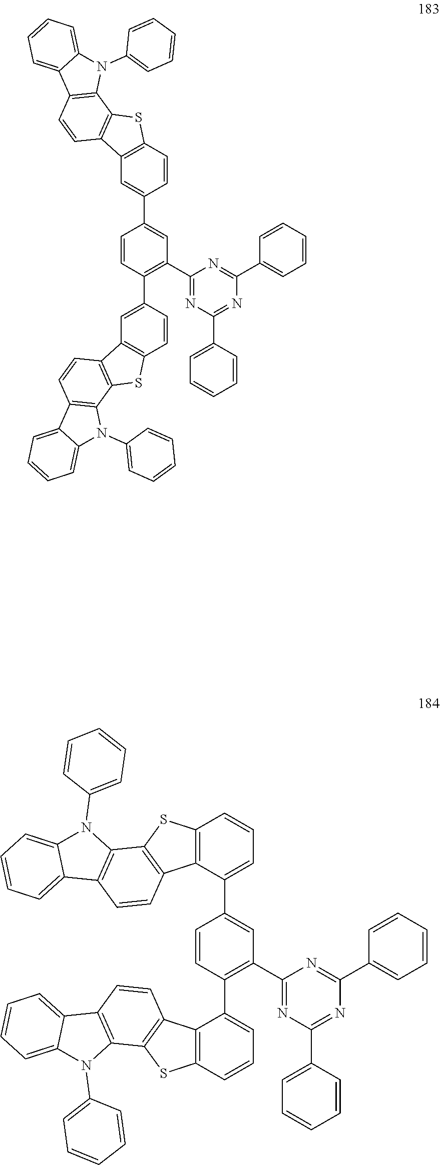 [SMILES: c1ccc(-c2nc(-c3ccccc3)nc(-c3cc(-c4ccc5sc6c(ccc7c8ccccc8n(-c8ccccc8)c76)c5c4)ccc3-c3ccc4sc5c(ccc6c7ccccc7n(-c7ccccc7)c65)c4c3)n2)cc1 ; c1ccc(-c2nc(-c3ccccc3)nc(-c3cc(-c4cccc5sc6c(ccc7c8ccccc8n(-c8ccccc8)c76)c45)ccc3-c3cccc4sc5c(ccc6c7ccccc7n(-c7ccccc7)c65)c34)n2)cc1]

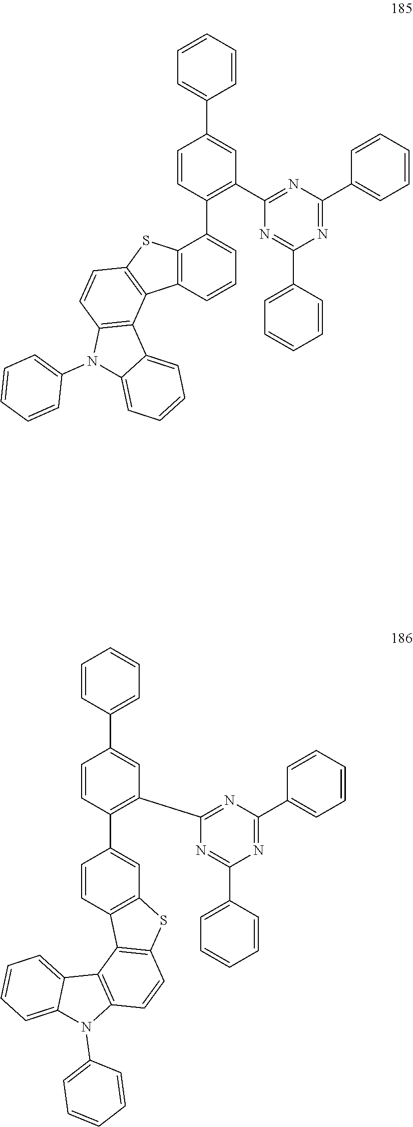 [SMILES: c1ccc(-c2ccc(-c3cccc4c3sc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4c(c3)sc3ccc5c(c6ccccc6n5-c5ccccc5)c34)c(-c3nc(-c4ccccc4)nc(-c4ccccc4)n3)c2)cc1]

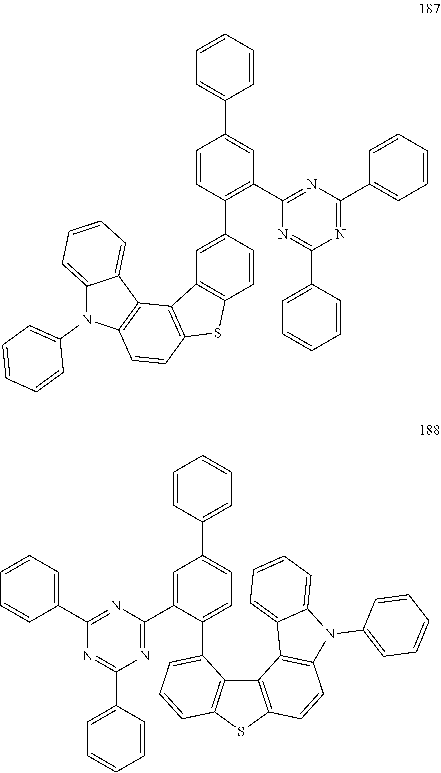 [SMILES: c1ccc(-c2ccc(-c3ccc4sc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4sc5ccc6c(c7ccccc7n6-c6ccccc6)c5c34)c(-c3nc(-c4ccccc4)nc(-c4ccccc4)n3)c2)cc1]

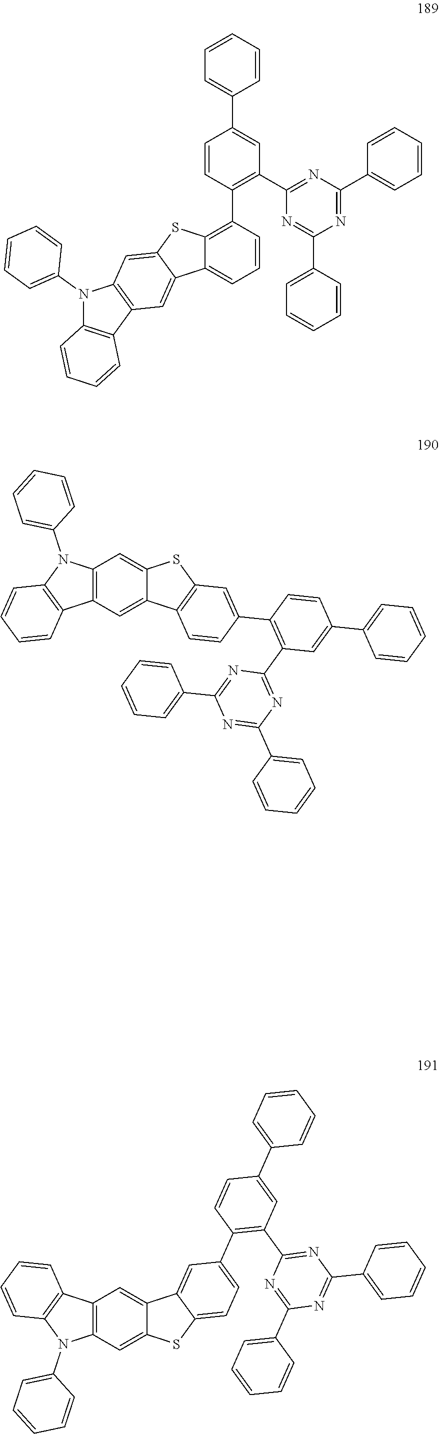 [SMILES: c1ccc(-c2ccc(-c3cccc4c3sc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)sc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4sc5cc6c(cc5c4c3)c3ccccc3n6-c3ccccc3)c(-c3nc(-c4ccccc4)nc(-c4ccccc4)n3)c2)cc1]

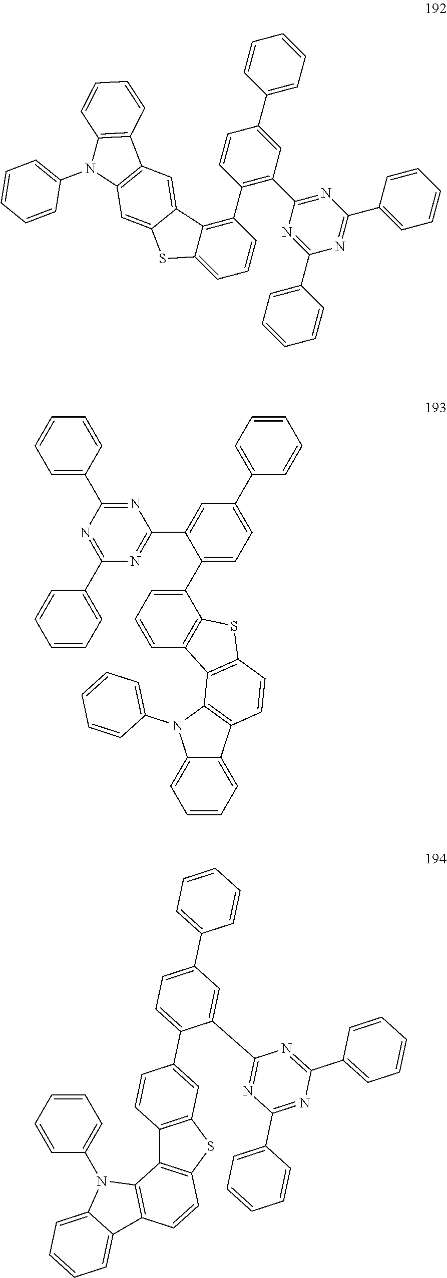 [SMILES: c1ccc(-c2ccc(-c3cccc4sc5cc6c(cc5c34)c3ccccc3n6-c3ccccc3)c(-c3nc(-c4ccccc4)nc(-c4ccccc4)n3)c2)cc1 ; c1ccc(-c2ccc(-c3cccc4c3sc3ccc5c6ccccc6n(-c6ccccc6)c5c34)c(-c3nc(-c4ccccc4)nc(-c4ccccc4)n3)c2)cc1 ; c1ccc(-c2ccc(-c3ccc4c(c3)sc3ccc5c6ccccc6n(-c6ccccc6)c5c34)c(-c3nc(-c4ccccc4)nc(-c4ccccc4)n3)c2)cc1]

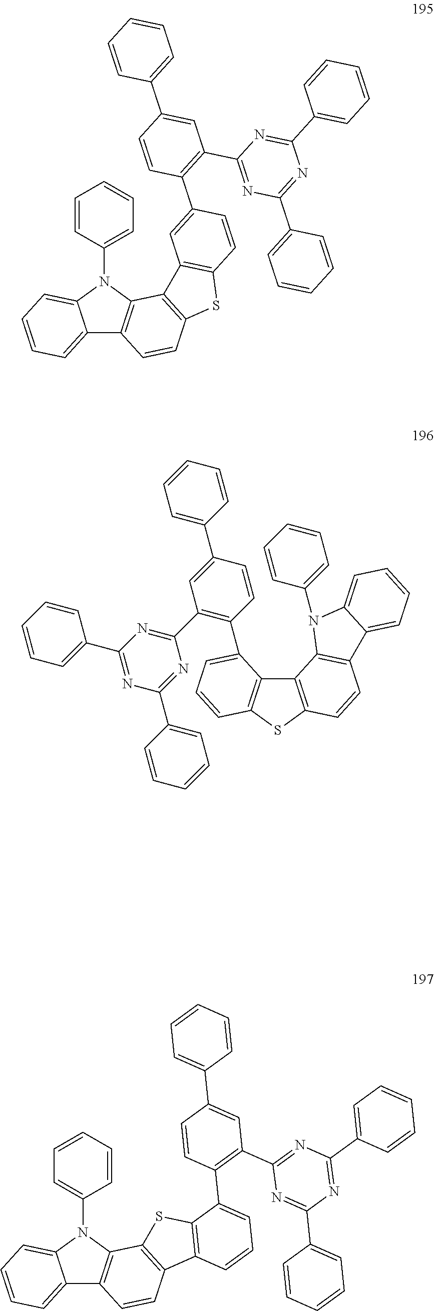 [SMILES: c1ccc(-c2ccc(-c3ccc4sc5ccc6c7ccccc7n(-c7ccccc7)c6c5c4c3)c(-c3nc(-c4ccccc4)nc(-c4ccccc4)n3)c2)cc1 ; c1ccc(-c2ccc(-c3cccc4sc5ccc6c7ccccc7n(-c7ccccc7)c6c5c34)c(-c3nc(-c4ccccc4)nc(-c4ccccc4)n3)c2)cc1 ; c1ccc(-c2ccc(-c3cccc4c3sc3c4ccc4c5ccccc5n(-c5ccccc5)c43)c(-c3nc(-c4ccccc4)nc(-c4ccccc4)n3)c2)cc1]

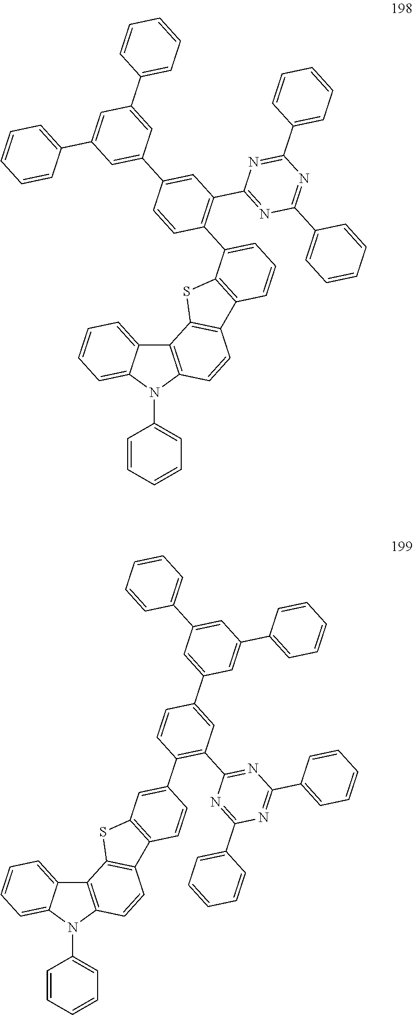 [SMILES: c1ccc(-c2cc(-c3ccccc3)cc(-c3ccc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1]

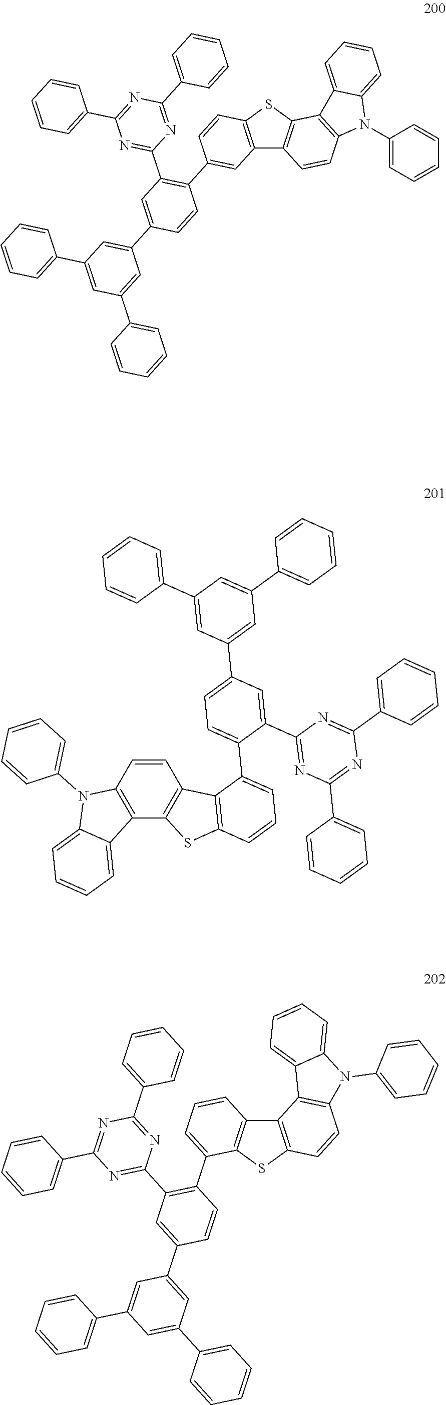 [SMILES: c1ccc(-c2cc(-c3ccccc3)cc(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4ccc6c(c7ccccc7n6-c6ccccc6)c45)c(-c4nc(-c5ccccc5)nc(-c5ccccc5)n4)c3)c2)cc1]

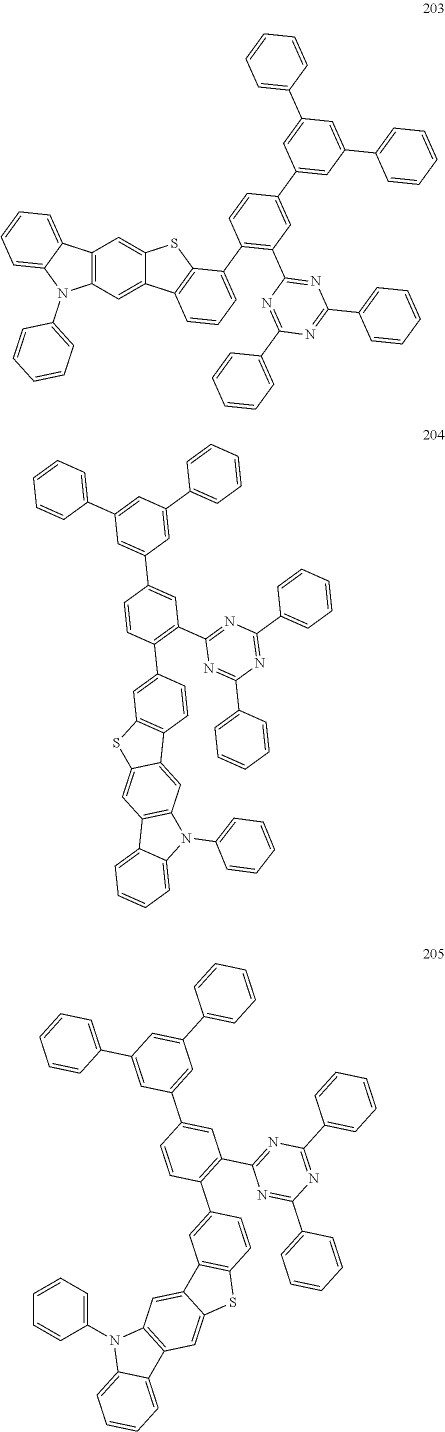

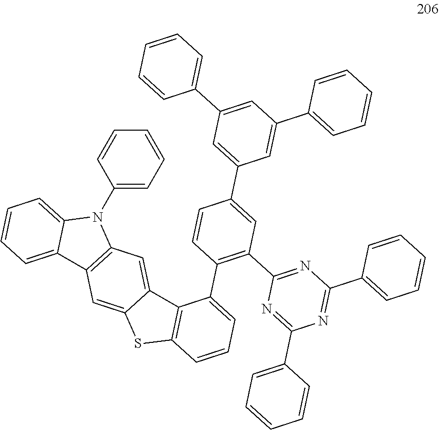 [SMILES: c1ccc(-c2cc(-c3ccccc3)cc(-c3ccc(-c4cccc5sc6cc7c8ccccc8n(-c8ccccc8)c7cc6c45)c(-c4nc(-c5ccccc5)nc(-c5ccccc5)n4)c3)c2)cc1]

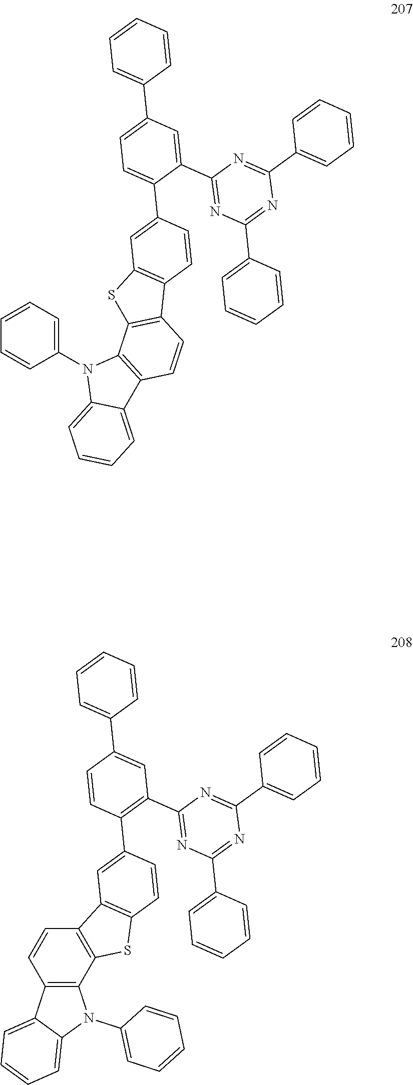 [SMILES: c1ccc(-c2ccc(-c3ccc4c(c3)sc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4sc5c(ccc6c7ccccc7n(-c7ccccc7)c65)c4c3)c(-c3nc(-c4ccccc4)nc(-c4ccccc4)n3)c2)cc1]

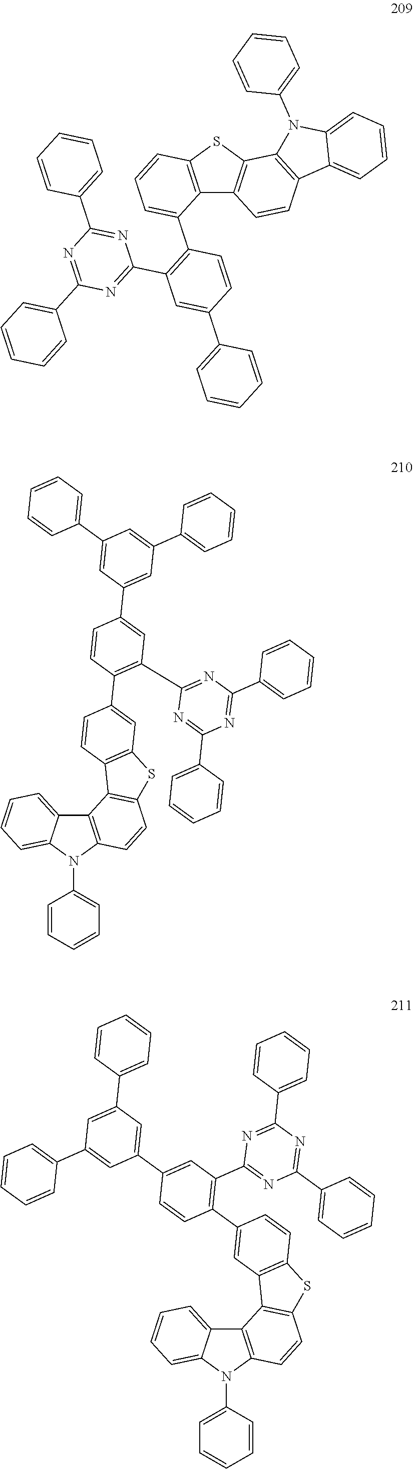 [SMILES: c1ccc(-c2ccc(-c3cccc4sc5c(ccc6c7ccccc7n(-c7ccccc7)c65)c34)c(-c3nc(-c4ccccc4)nc(-c4ccccc4)n3)c2)cc1 ; c1ccc(-c2cc(-c3ccccc3)cc(-c3ccc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1]

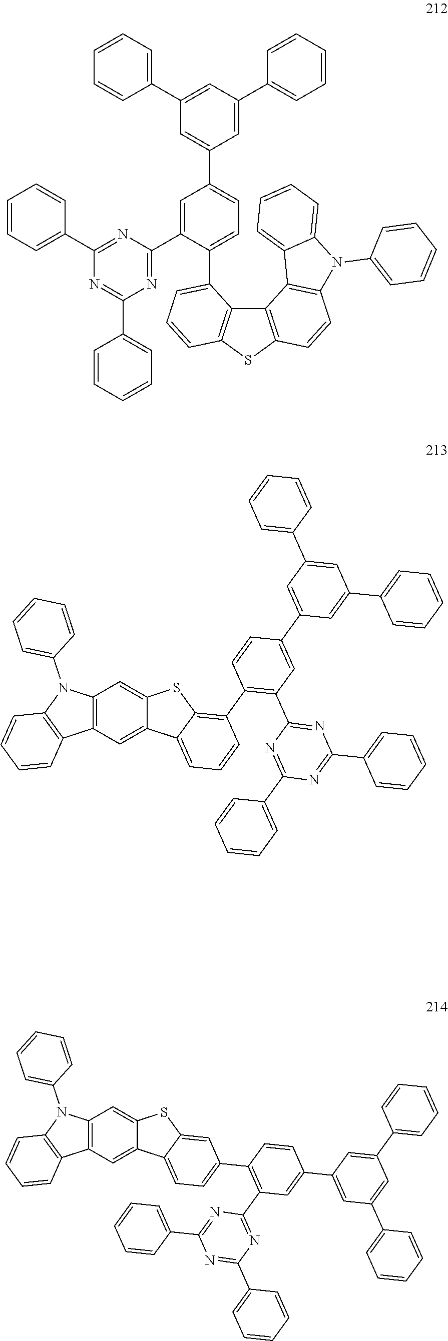 [SMILES: c1ccc(-c2cc(-c3ccccc3)cc(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1]

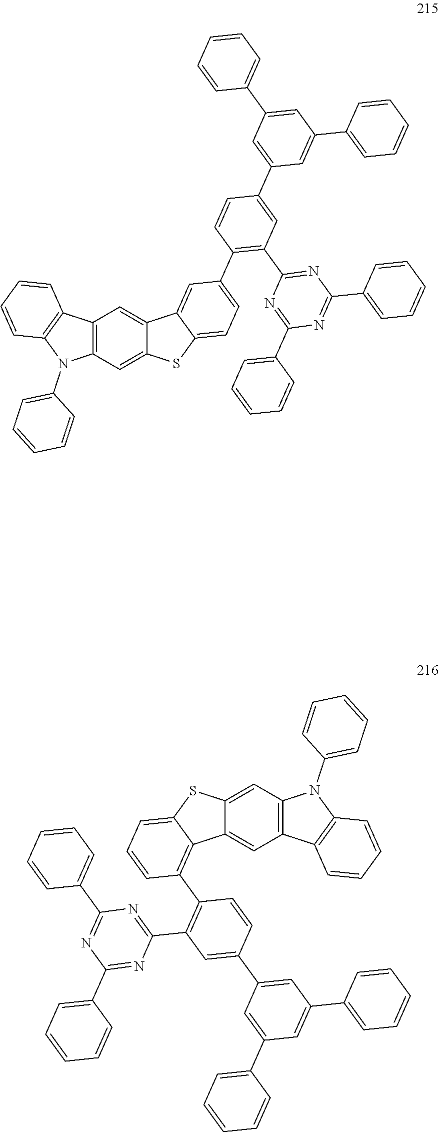 [SMILES: c1ccc(-c2cc(-c3ccccc3)cc(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5sc6cc7c(cc6c45)c4ccccc4n7-c4ccccc4)c(-c4nc(-c5ccccc5)nc(-c5ccccc5)n4)c3)c2)cc1]

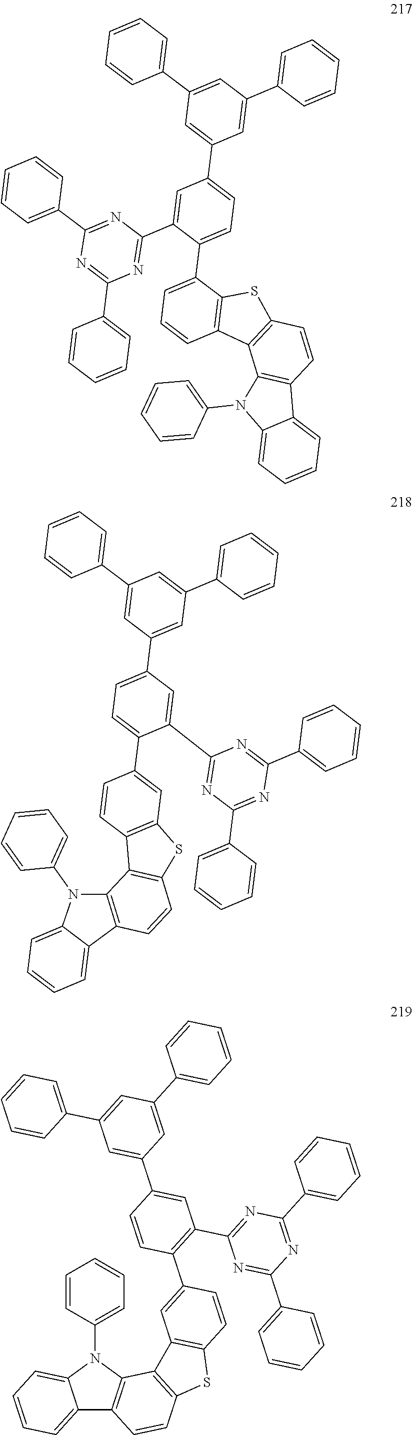 [SMILES: c1ccc(-c2cc(-c3ccccc3)cc(-c3ccc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5sc6ccc7c8ccccc8n(-c8ccccc8)c7c6c5c4)c(-c4nc(-c5ccccc5)nc(-c5ccccc5)n4)c3)c2)cc1]

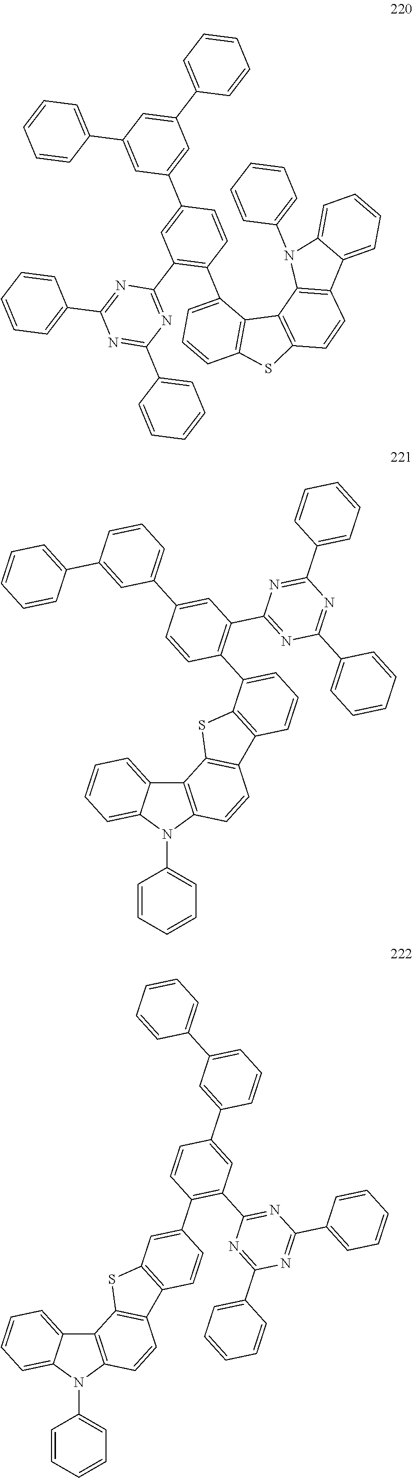

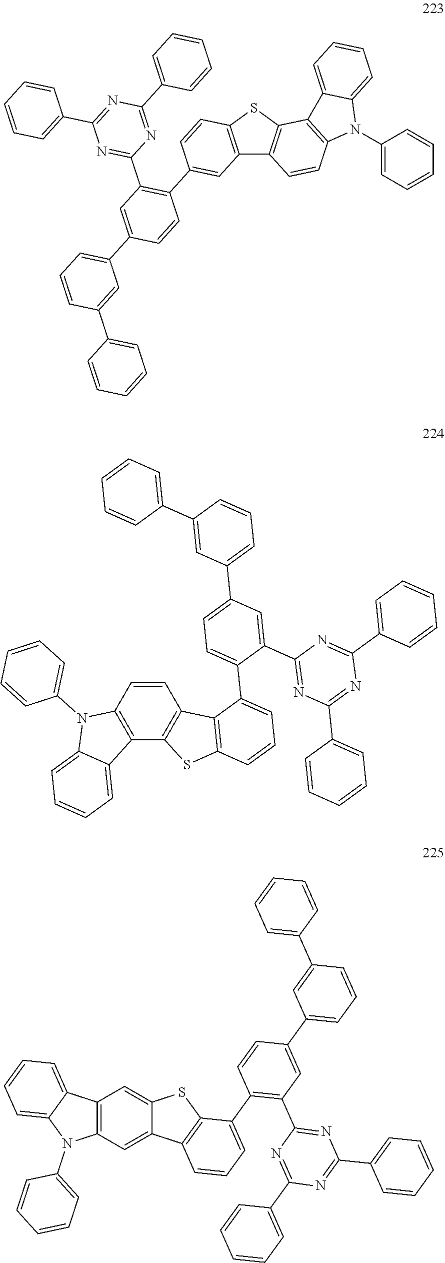 [SMILES: c1ccc(-c2cccc(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3ccc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cccc(-c3ccc(-c4cccc5c4sc4cc6c7ccccc7n(-c7ccccc7)c6cc45)c(-c4nc(-c5ccccc5)nc(-c5ccccc5)n4)c3)c2)cc1]

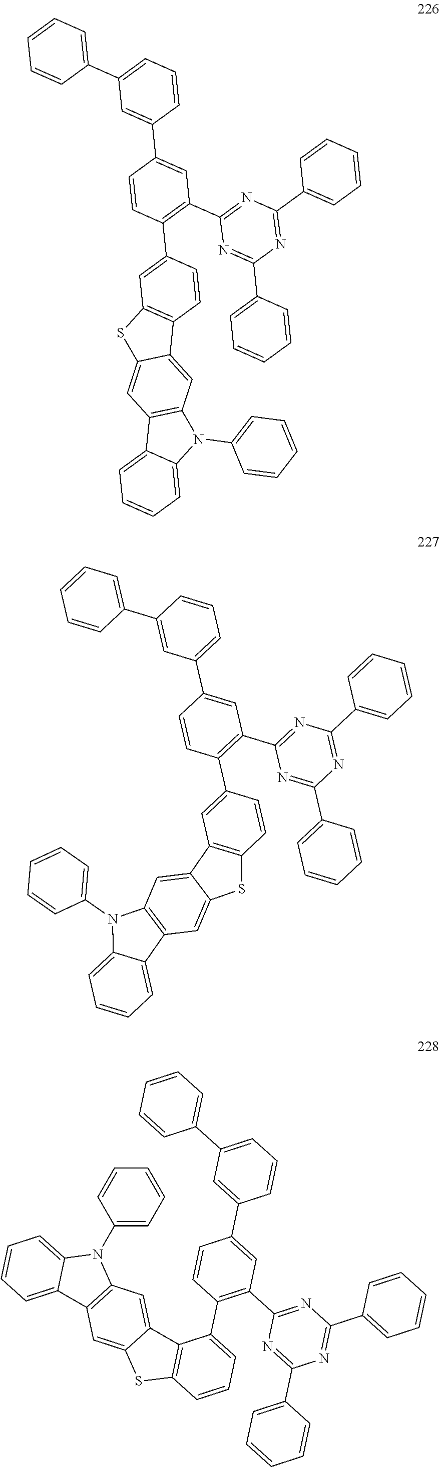 [SMILES: c1ccc(-c2cccc(-c3ccc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3ccc(-c4cccc5sc6cc7c8ccccc8n(-c8ccccc8)c7cc6c45)c(-c4nc(-c5ccccc5)nc(-c5ccccc5)n4)c3)c2)cc1]

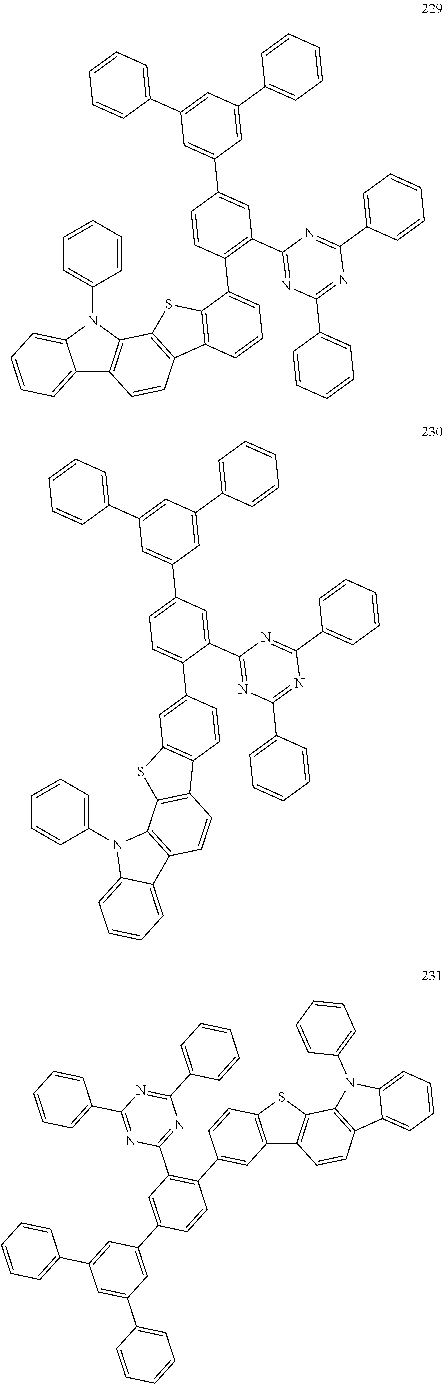 [SMILES: c1ccc(-c2cc(-c3ccccc3)cc(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1]

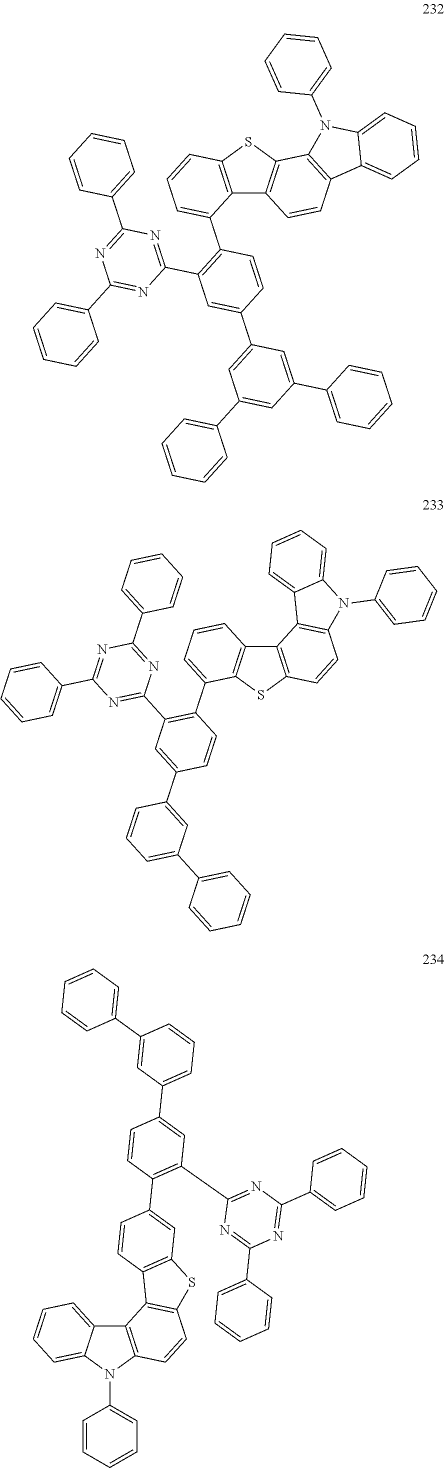 [SMILES: c1ccc(-c2cc(-c3ccccc3)cc(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3ccc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2cccc(-c3ccc(-c4ccc5c(c4)sc4ccc6c(c7ccccc7n6-c6ccccc6)c45)c(-c4nc(-c5ccccc5)nc(-c5ccccc5)n4)c3)c2)cc1]

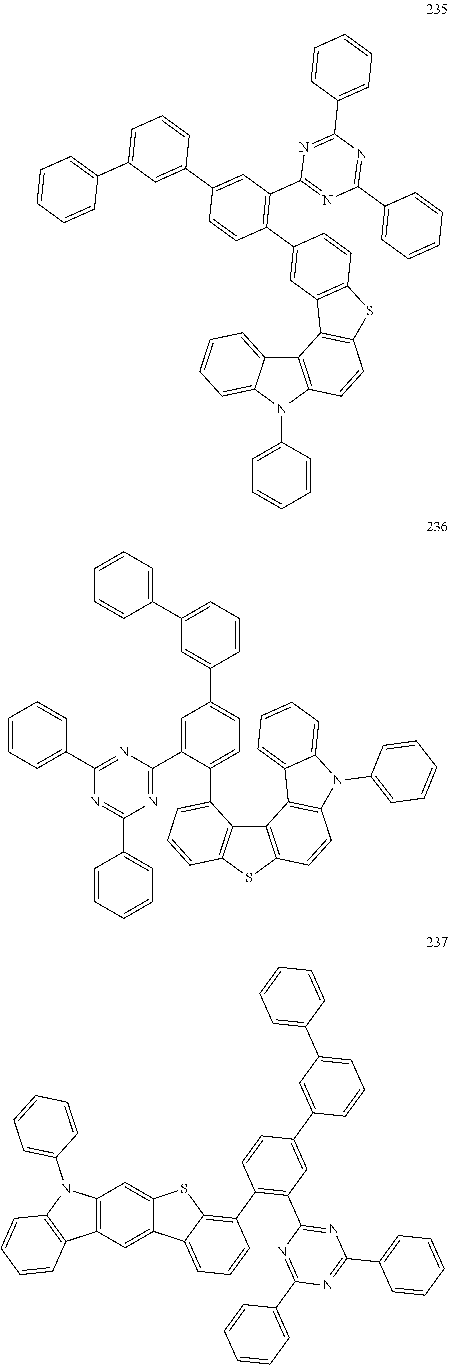 [SMILES: c1ccc(-c2cccc(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3ccc(-c4cccc5c4sc4cc6c(cc45)c4ccccc4n6-c4ccccc4)c(-c4nc(-c5ccccc5)nc(-c5ccccc5)n4)c3)c2)cc1]

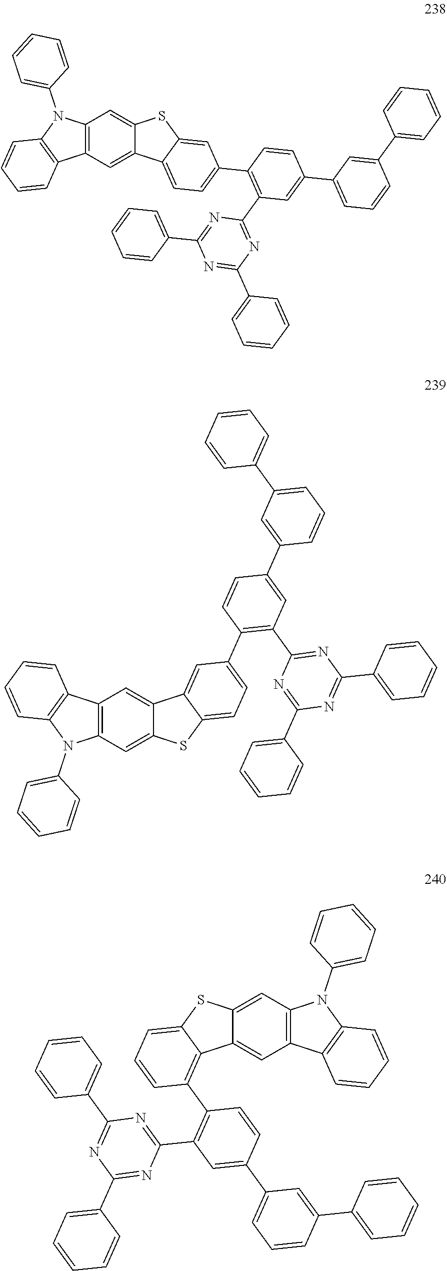 [SMILES: c1ccc(-c2cccc(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5sc6cc7c(cc6c45)c4ccccc4n7-c4ccccc4)c(-c4nc(-c5ccccc5)nc(-c5ccccc5)n4)c3)c2)cc1]

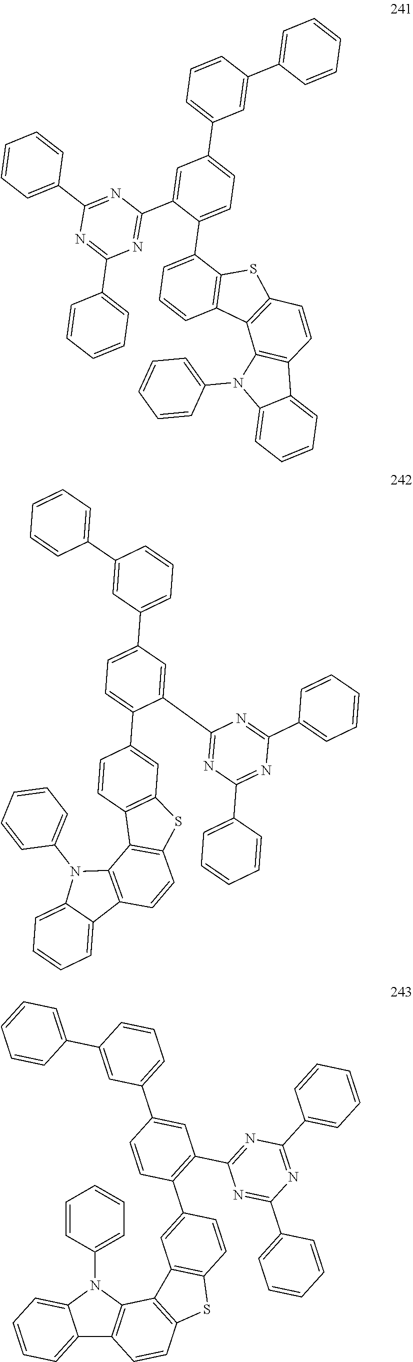

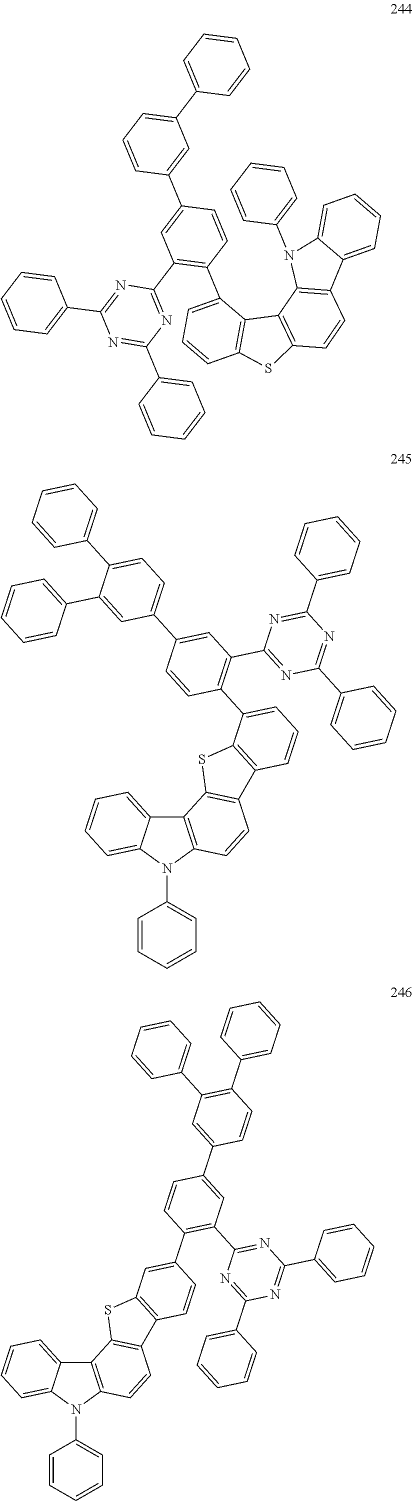 [SMILES: c1ccc(-c2cccc(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3sc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)sc3c4ccc4c3c3ccccc3n4-c3ccccc3)n2)cc1]

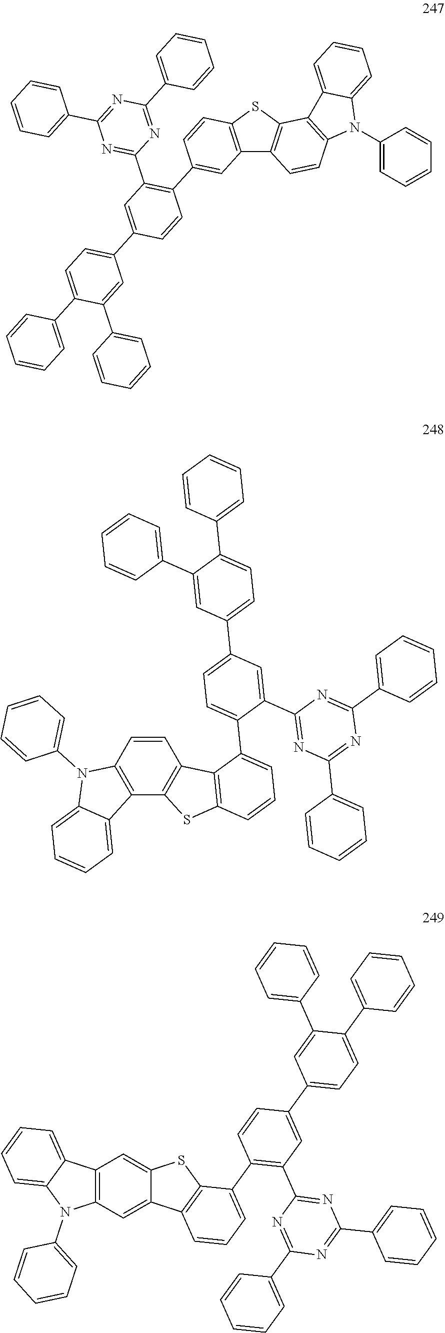 [SMILES: c1ccc(-c2nc(-c3ccccc3)nc(-c3cc(-c4ccc(-c5ccccc5)c(-c5ccccc5)c4)ccc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4sc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)ccc3-c3cccc4c3sc3cc5c6ccccc6n(-c6ccccc6)c5cc34)n2)cc1]

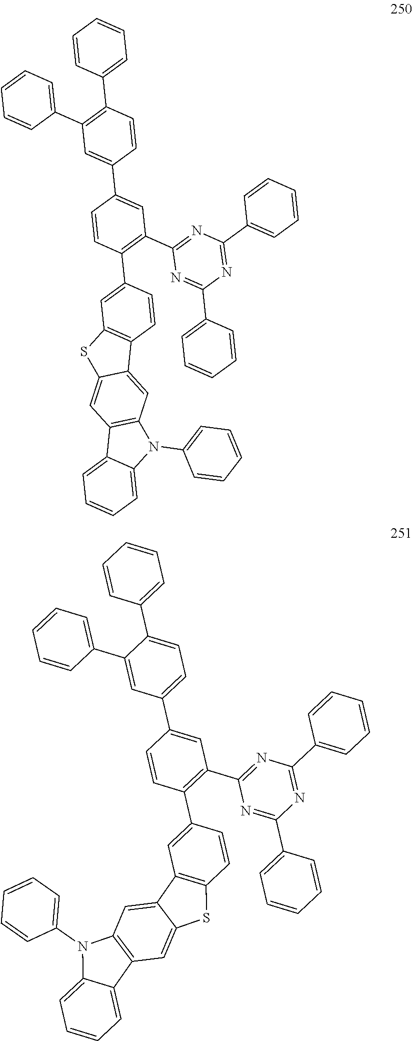

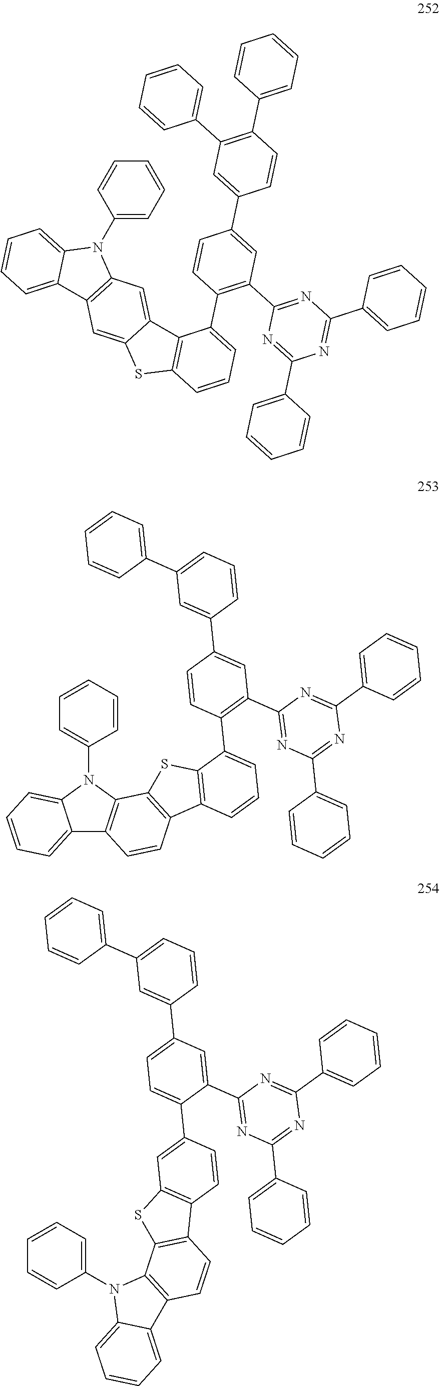 [SMILES: c1ccc(-c2nc(-c3ccccc3)nc(-c3cc(-c4ccc(-c5ccccc5)c(-c5ccccc5)c4)ccc3-c3cccc4sc5cc6c7ccccc7n(-c7ccccc7)c6cc5c34)n2)cc1 ; c1ccc(-c2cccc(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1]

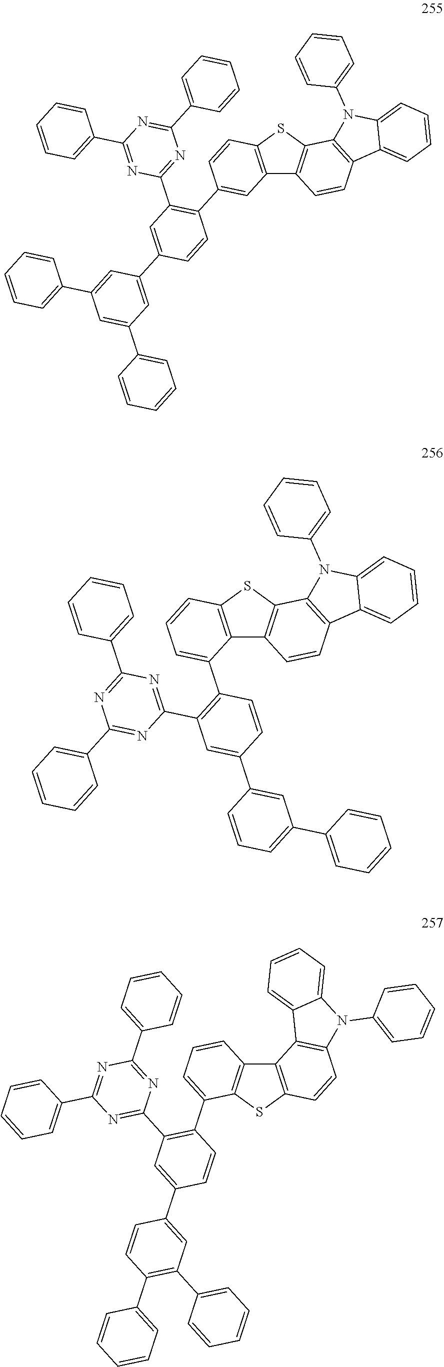 [SMILES: c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3sc3ccc5c(c6ccccc6n5-c5ccccc5)c34)n2)cc1]

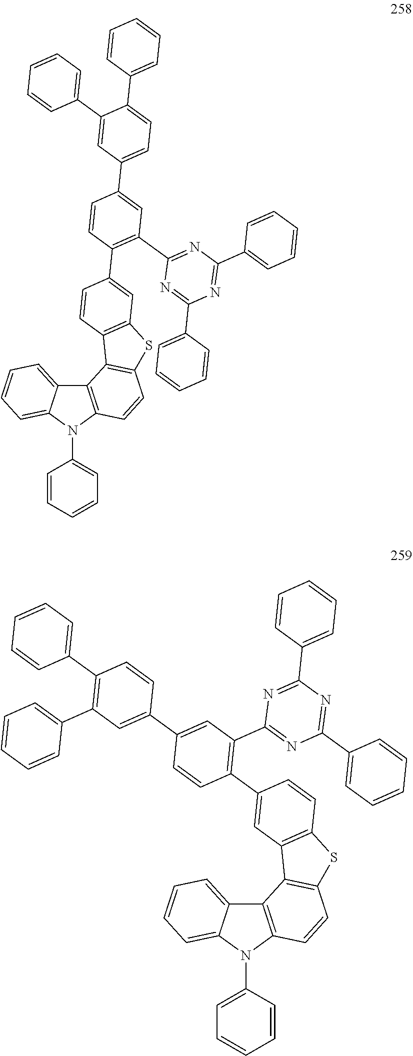 [SMILES: c1ccc(-c2nc(-c3ccccc3)nc(-c3cc(-c4ccc(-c5ccccc5)c(-c5ccccc5)c4)ccc3-c3ccc4c(c3)sc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)ccc3-c3ccc4sc5ccc6c(c7ccccc7n6-c6ccccc6)c5c4c3)n2)cc1]

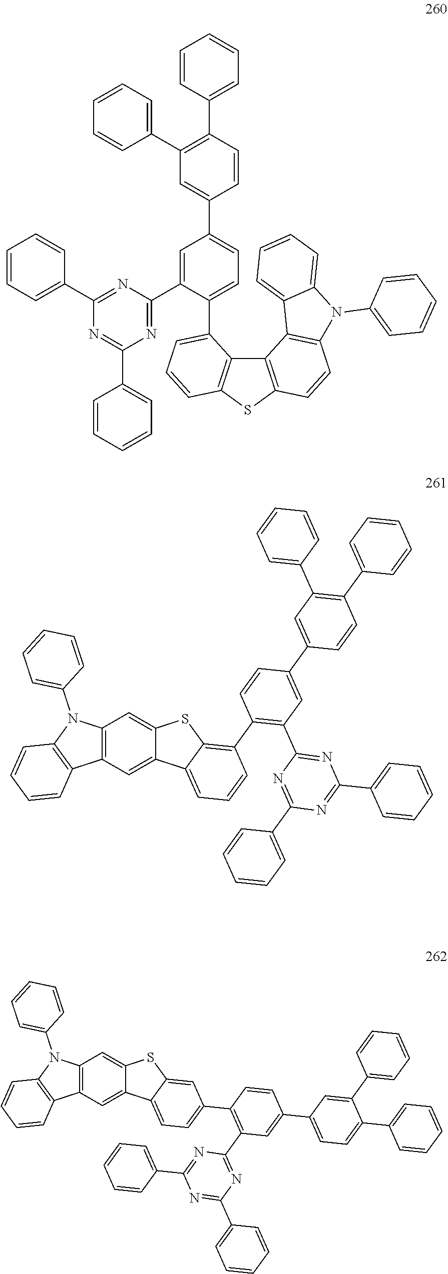 [SMILES: c1ccc(-c2nc(-c3ccccc3)nc(-c3cc(-c4ccc(-c5ccccc5)c(-c5ccccc5)c4)ccc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3cccc4c3sc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)sc3cc5c(cc34)c3ccccc3n5-c3ccccc3)n2)cc1]

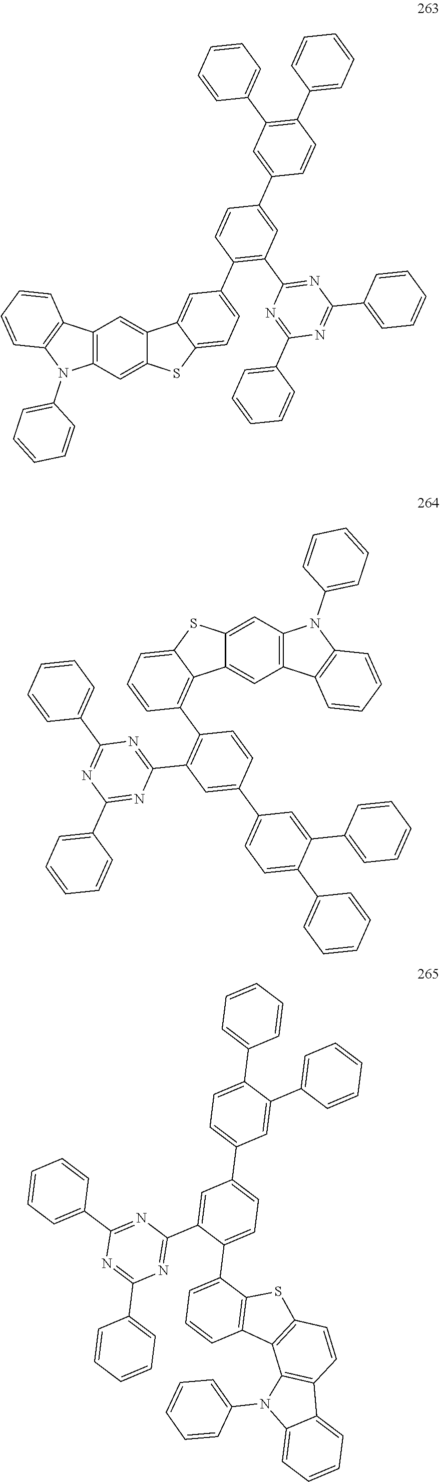 [SMILES: c1ccc(-c2nc(-c3ccccc3)nc(-c3cc(-c4ccc(-c5ccccc5)c(-c5ccccc5)c4)ccc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4sc5cc6c(cc5c34)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4c3sc3ccc5c6ccccc6n(-c6ccccc6)c5c34)n2)cc1]

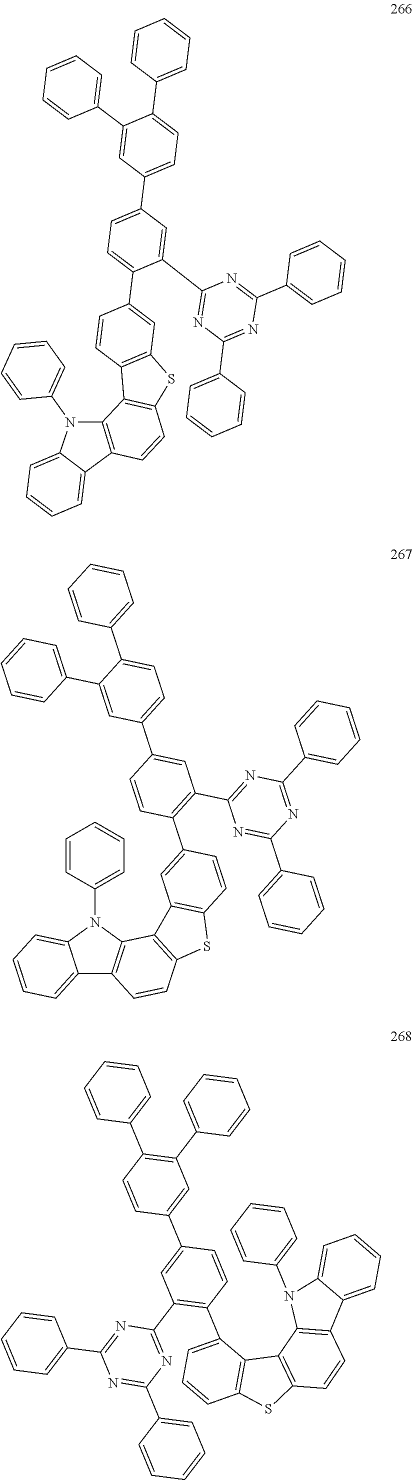 [SMILES: c1ccc(-c2nc(-c3ccccc3)nc(-c3cc(-c4ccc(-c5ccccc5)c(-c5ccccc5)c4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3ccc4sc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)ccc3-c3cccc4sc5ccc6c7ccccc7n(-c7ccccc7)c6c5c34)n2)cc1]

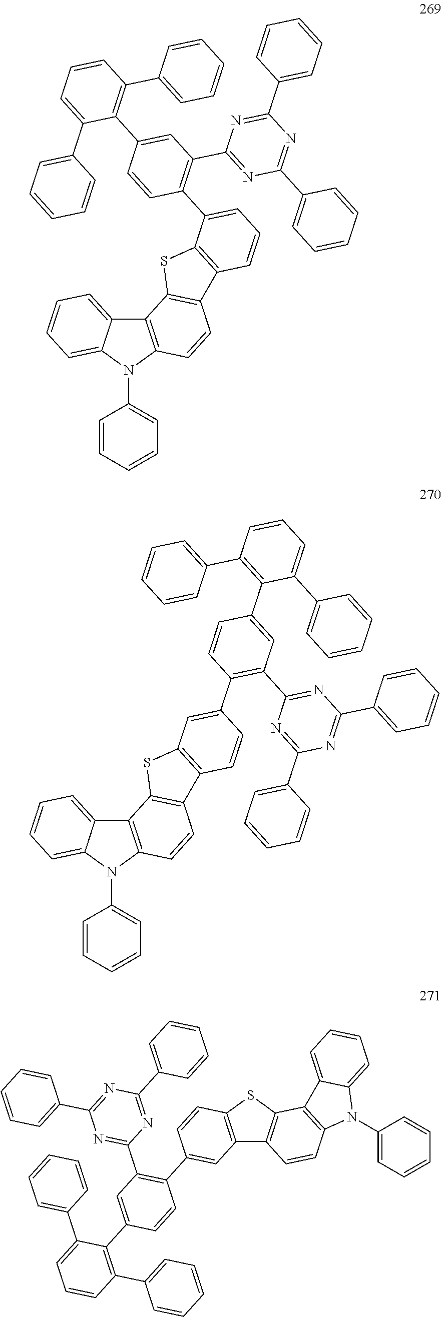 [SMILES: c1ccc(-c2nc(-c3ccccc3)nc(-c3cc(-c4c(-c5ccccc5)cccc4-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4sc5c(ccc6c5c5ccccc5n6-c5ccccc5)c4c3)n2)cc1]

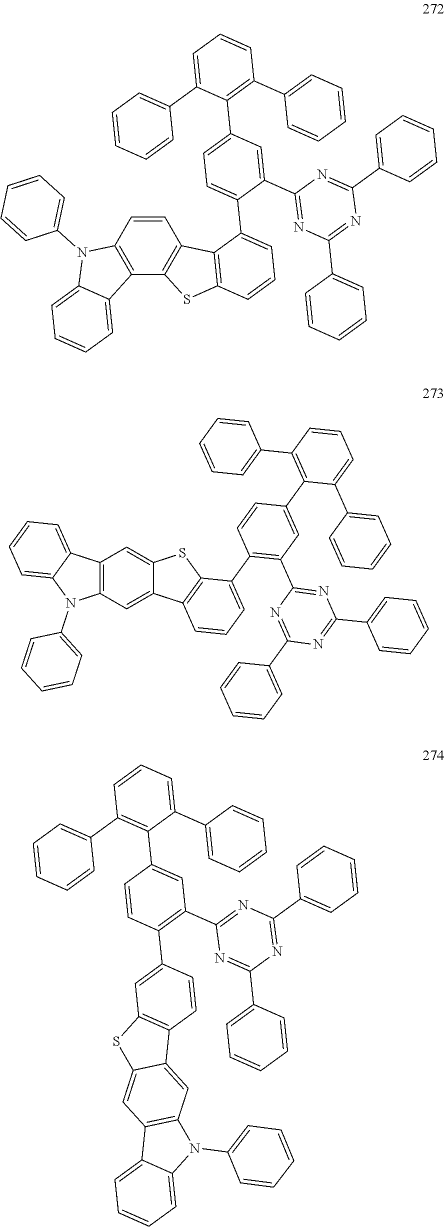 [SMILES: c1ccc(-c2nc(-c3ccccc3)nc(-c3cc(-c4c(-c5ccccc5)cccc4-c4ccccc4)ccc3-c3cccc4sc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)ccc3-c3cccc4c3sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4c(c3)sc3cc5c6ccccc6n(-c6ccccc6)c5cc34)n2)cc1]

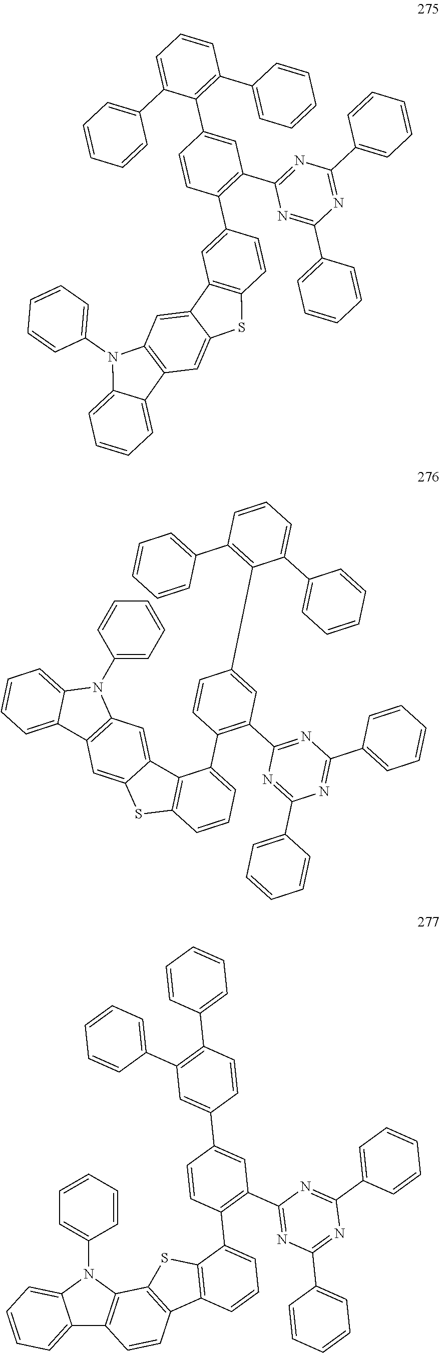 [SMILES: c1ccc(-c2nc(-c3ccccc3)nc(-c3cc(-c4c(-c5ccccc5)cccc4-c4ccccc4)ccc3-c3ccc4sc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)ccc3-c3cccc4sc5cc6c7ccccc7n(-c7ccccc7)c6cc5c34)n2)cc1 ; c1ccc(-c2nc(-c3ccccc3)nc(-c3cc(-c4ccc(-c5ccccc5)c(-c5ccccc5)c4)ccc3-c3cccc4c3sc3c4ccc4c5ccccc5n(-c5ccccc5)c43)n2)cc1]

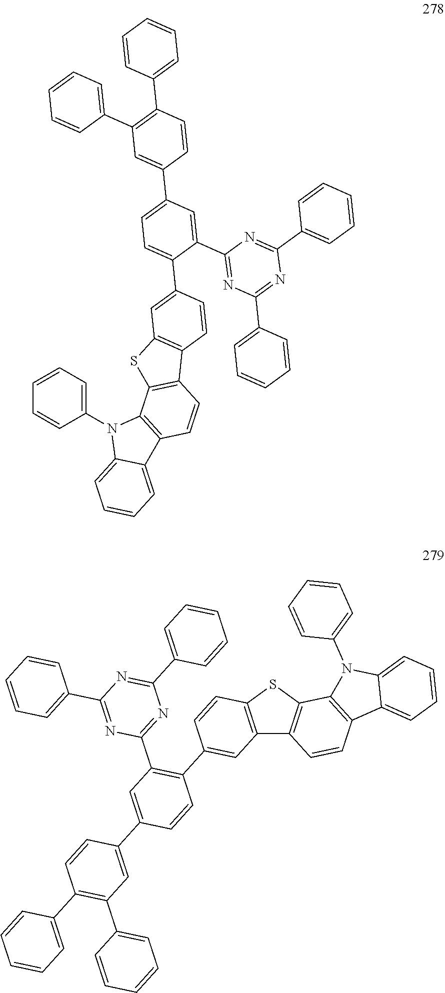 [SMILES: c1ccc(-c2nc(-c3ccccc3)nc(-c3cc(-c4ccc(-c5ccccc5)c(-c5ccccc5)c4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4sc5c(ccc6c7ccccc7n(-c7ccccc7)c65)c4c3)n2)cc1]

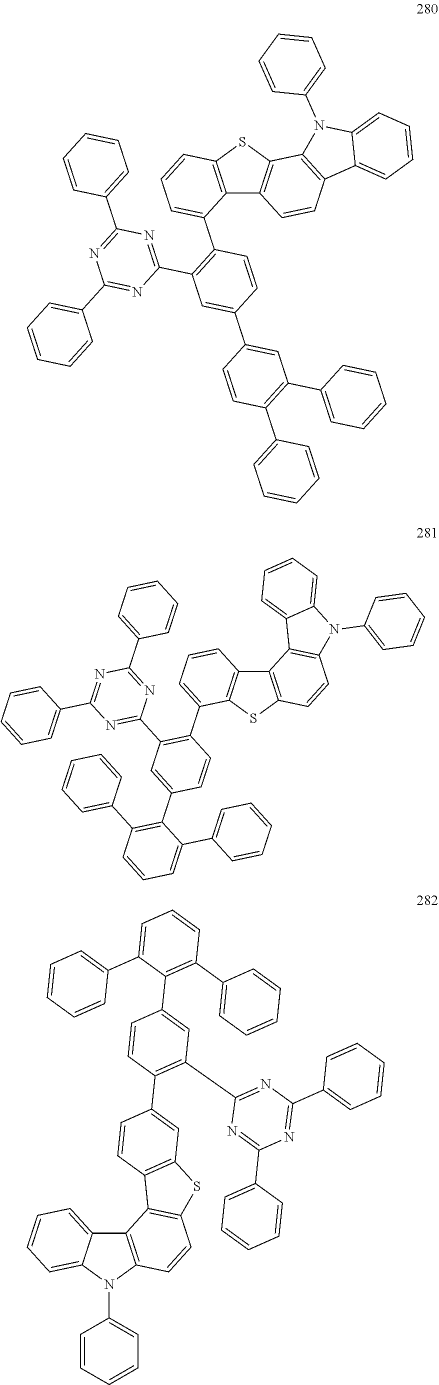

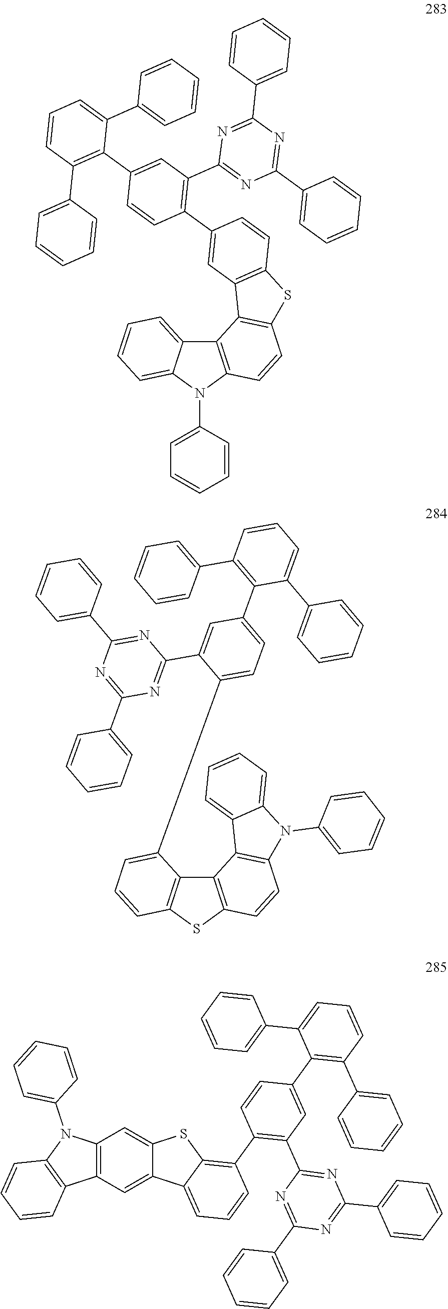 [SMILES: c1ccc(-c2nc(-c3ccccc3)nc(-c3cc(-c4c(-c5ccccc5)cccc4-c4ccccc4)ccc3-c3ccc4sc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)ccc3-c3cccc4sc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)ccc3-c3cccc4c3sc3cc5c(cc34)c3ccccc3n5-c3ccccc3)n2)cc1]

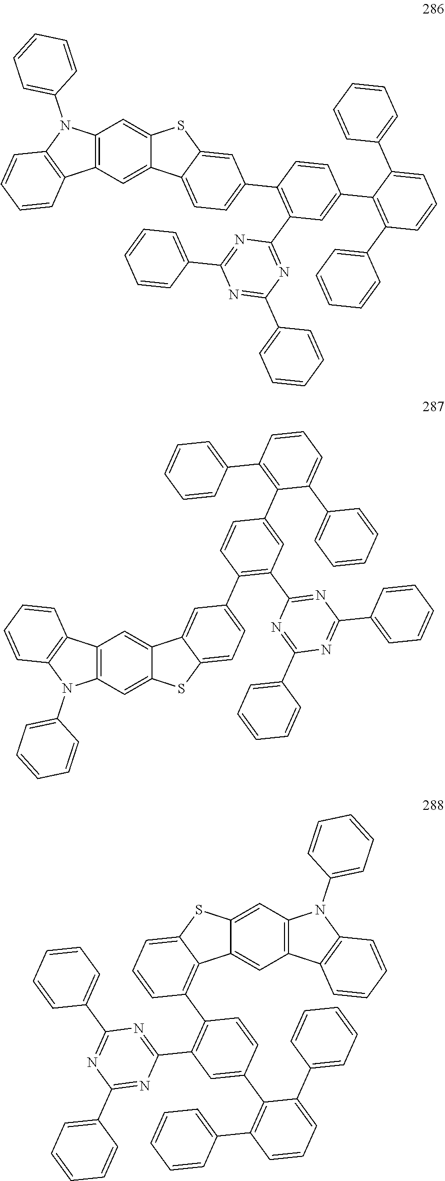 [SMILES: c1ccc(-c2nc(-c3ccccc3)nc(-c3cc(-c4c(-c5ccccc5)cccc4-c4ccccc4)ccc3-c3ccc4c(c3)sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)ccc3-c3ccc4sc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)ccc3-c3cccc4sc5cc6c(cc5c34)c3ccccc3n6-c3ccccc3)n2)cc1]

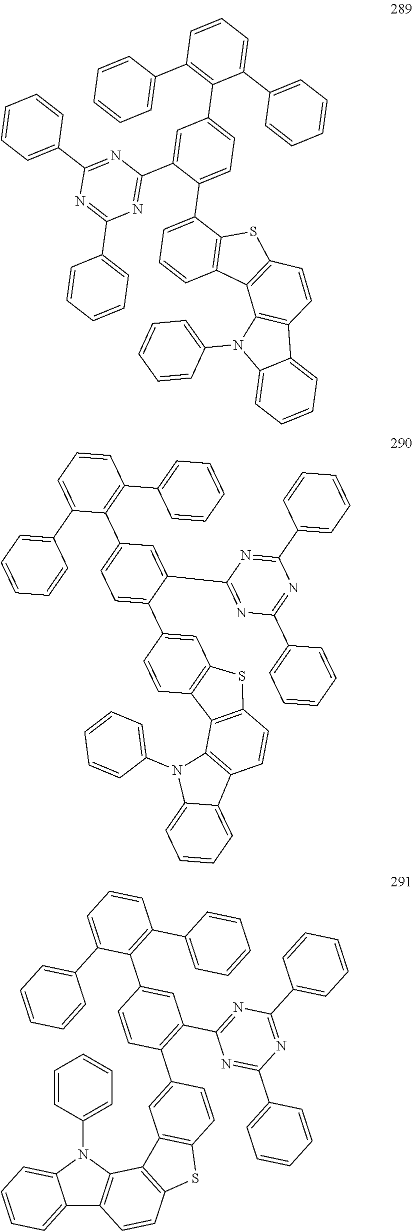 [SMILES: c1ccc(-c2nc(-c3ccccc3)nc(-c3cc(-c4c(-c5ccccc5)cccc4-c4ccccc4)ccc3-c3cccc4c3sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)ccc3-c3ccc4sc5ccc6c7ccccc7n(-c7ccccc7)c6c5c4c3)n2)cc1]

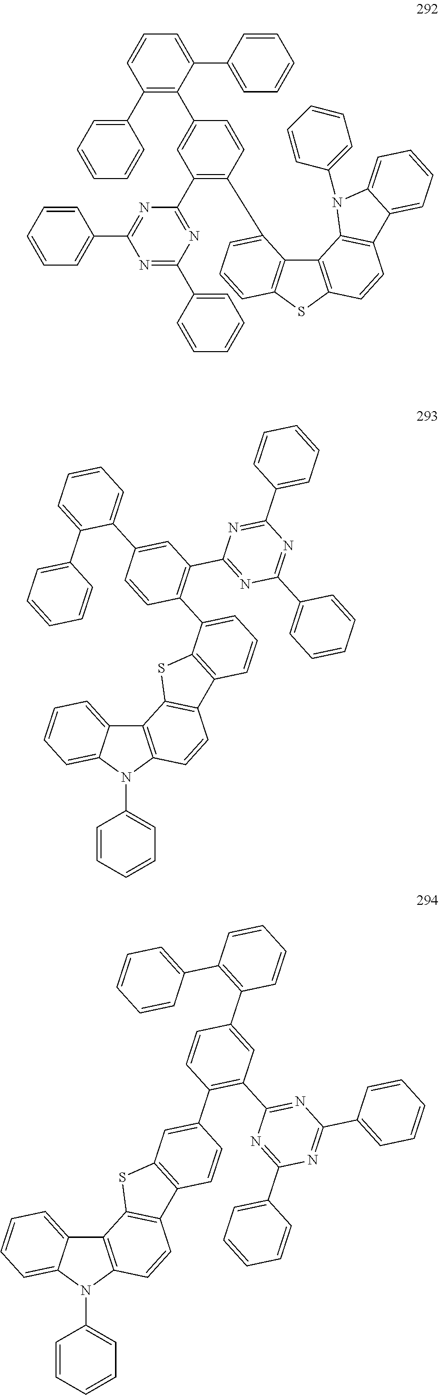 [SMILES: c1ccc(-c2nc(-c3ccccc3)nc(-c3cc(-c4c(-c5ccccc5)cccc4-c4ccccc4)ccc3-c3cccc4sc5ccc6c7ccccc7n(-c7ccccc7)c6c5c34)n2)cc1 ; c1ccc(-c2nc(-c3ccccc3)nc(-c3cc(-c4ccccc4-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4ccccc4-c4ccccc4)ccc3-c3ccc4c(c3)sc3c4ccc4c3c3ccccc3n4-c3ccccc3)n2)cc1]

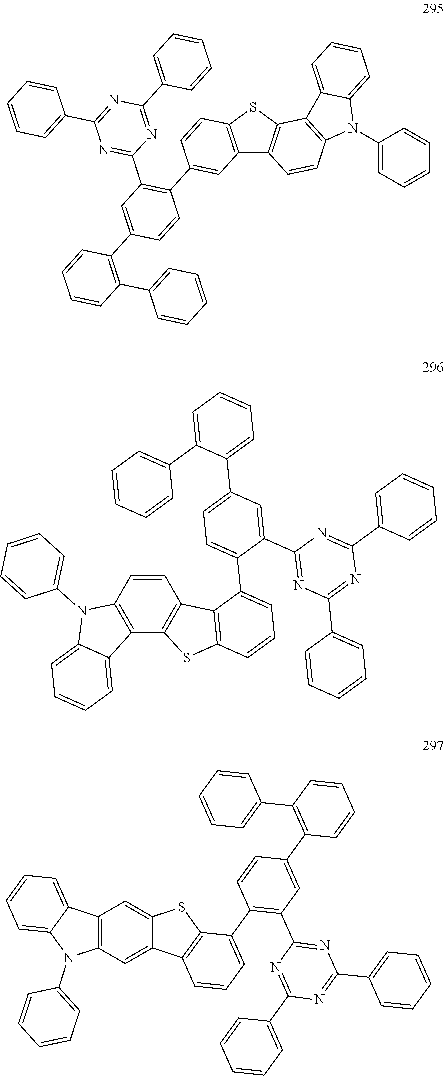 [SMILES: c1ccc(-c2nc(-c3ccccc3)nc(-c3cc(-c4ccccc4-c4ccccc4)ccc3-c3ccc4sc5c(ccc6c5c5ccccc5n6-c5ccccc5)c4c3)n2)cc1 ; c1ccc(-c2nc(-c3ccccc3)nc(-c3cc(-c4ccccc4-c4ccccc4)ccc3-c3cccc4sc5c(ccc6c5c5ccccc5n6-c5ccccc5)c34)n2)cc1 ; c1ccc(-c2nc(-c3ccccc3)nc(-c3cc(-c4ccccc4-c4ccccc4)ccc3-c3cccc4c3sc3cc5c6ccccc6n(-c6ccccc6)c5cc34)n2)cc1]

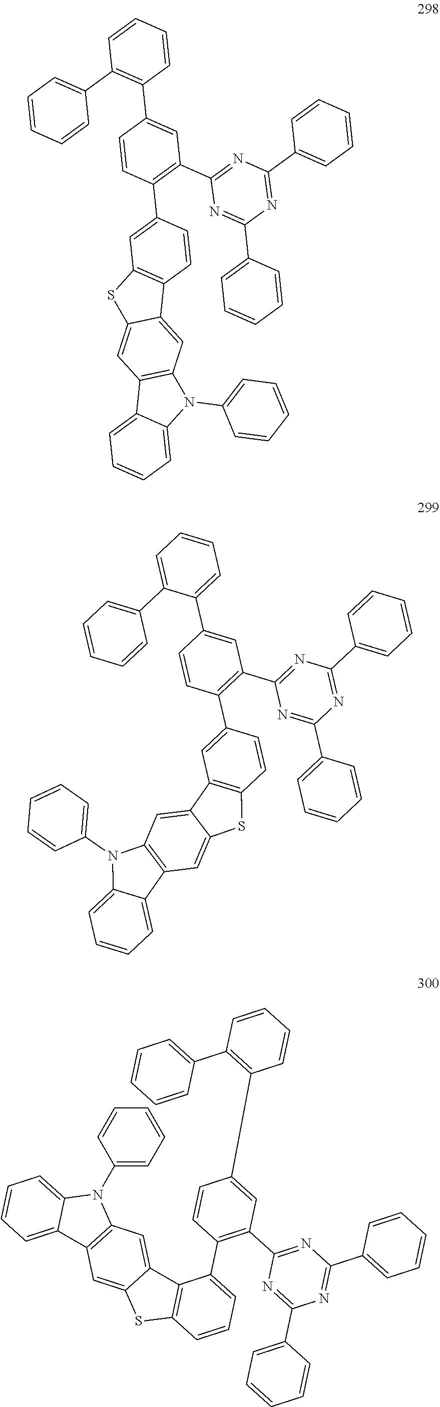 [SMILES: c1ccc(-c2nc(-c3ccccc3)nc(-c3cc(-c4ccccc4-c4ccccc4)ccc3-c3ccc4c(c3)sc3cc5c6ccccc6n(-c6ccccc6)c5cc34)n2)cc1 ; c1ccc(-c2nc(-c3ccccc3)nc(-c3cc(-c4ccccc4-c4ccccc4)ccc3-c3ccc4sc5cc6c7ccccc7n(-c7ccccc7)c6cc5c4c3)n2)cc1 ; c1ccc(-c2nc(-c3ccccc3)nc(-c3cc(-c4ccccc4-c4ccccc4)ccc3-c3cccc4sc5cc6c7ccccc7n(-c7ccccc7)c6cc5c34)n2)cc1]

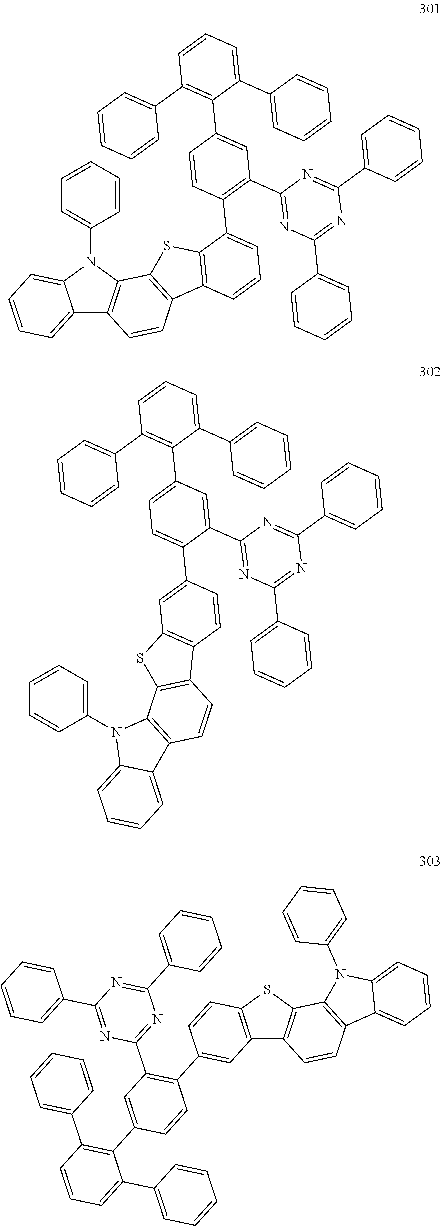 [SMILES: c1ccc(-c2nc(-c3ccccc3)nc(-c3cc(-c4c(-c5ccccc5)cccc4-c4ccccc4)ccc3-c3cccc4c3sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4sc5c(ccc6c7ccccc7n(-c7ccccc7)c65)c4c3)n2)cc1]

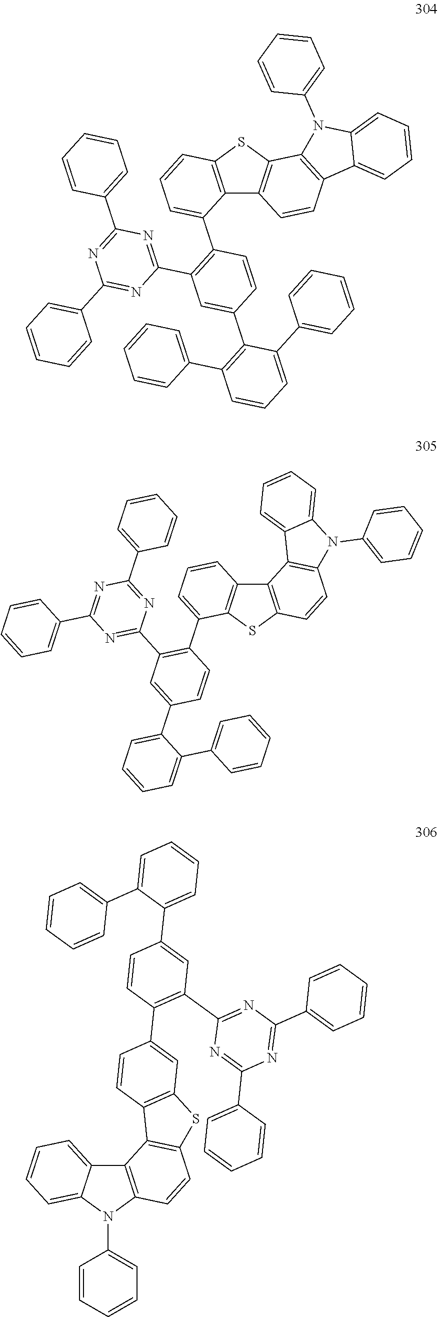 [SMILES: c1ccc(-c2nc(-c3ccccc3)nc(-c3cc(-c4c(-c5ccccc5)cccc4-c4ccccc4)ccc3-c3cccc4sc5c(ccc6c7ccccc7n(-c7ccccc7)c65)c34)n2)cc1 ; c1ccc(-c2nc(-c3ccccc3)nc(-c3cc(-c4ccccc4-c4ccccc4)ccc3-c3cccc4c3sc3ccc5c(c6ccccc6n5-c5ccccc5)c34)n2)cc1 ; c1ccc(-c2nc(-c3ccccc3)nc(-c3cc(-c4ccccc4-c4ccccc4)ccc3-c3ccc4c(c3)sc3ccc5c(c6ccccc6n5-c5ccccc5)c34)n2)cc1]

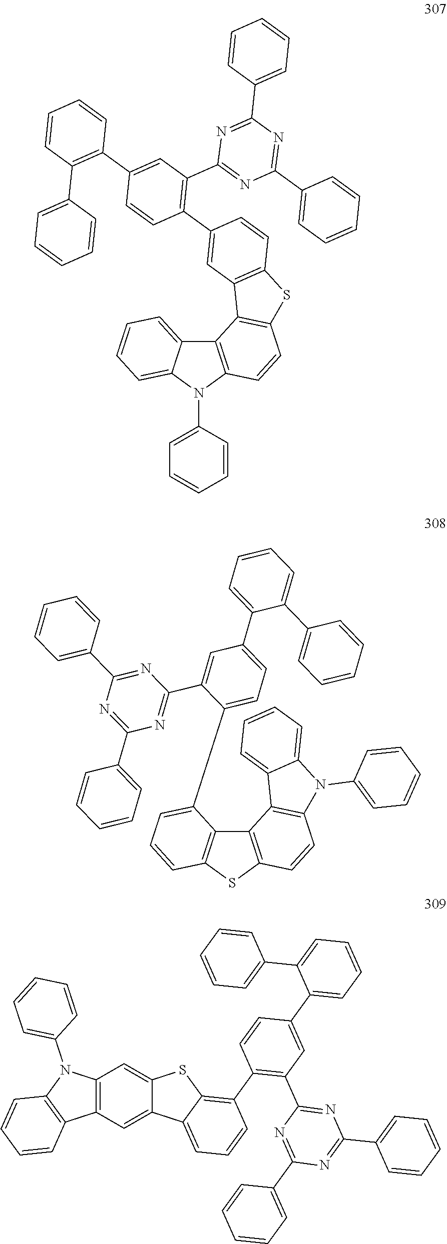 [SMILES: c1ccc(-c2nc(-c3ccccc3)nc(-c3cc(-c4ccccc4-c4ccccc4)ccc3-c3ccc4sc5ccc6c(c7ccccc7n6-c6ccccc6)c5c4c3)n2)cc1 ; c1ccc(-c2nc(-c3ccccc3)nc(-c3cc(-c4ccccc4-c4ccccc4)ccc3-c3cccc4sc5ccc6c(c7ccccc7n6-c6ccccc6)c5c34)n2)cc1 ; c1ccc(-c2nc(-c3ccccc3)nc(-c3cc(-c4ccccc4-c4ccccc4)ccc3-c3cccc4c3sc3cc5c(cc34)c3ccccc3n5-c3ccccc3)n2)cc1]

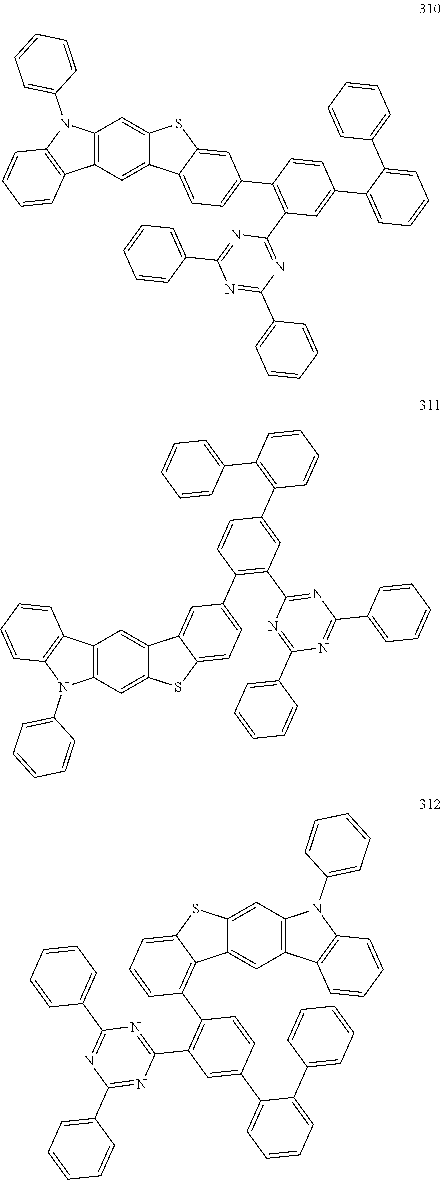 [SMILES: c1ccc(-c2nc(-c3ccccc3)nc(-c3cc(-c4ccccc4-c4ccccc4)ccc3-c3ccc4c(c3)sc3cc5c(cc34)c3ccccc3n5-c3ccccc3)n2)cc1 ; c1ccc(-c2nc(-c3ccccc3)nc(-c3cc(-c4ccccc4-c4ccccc4)ccc3-c3ccc4sc5cc6c(cc5c4c3)c3ccccc3n6-c3ccccc3)n2)cc1 ; c1ccc(-c2nc(-c3ccccc3)nc(-c3cc(-c4ccccc4-c4ccccc4)ccc3-c3cccc4sc5cc6c(cc5c34)c3ccccc3n6-c3ccccc3)n2)cc1]

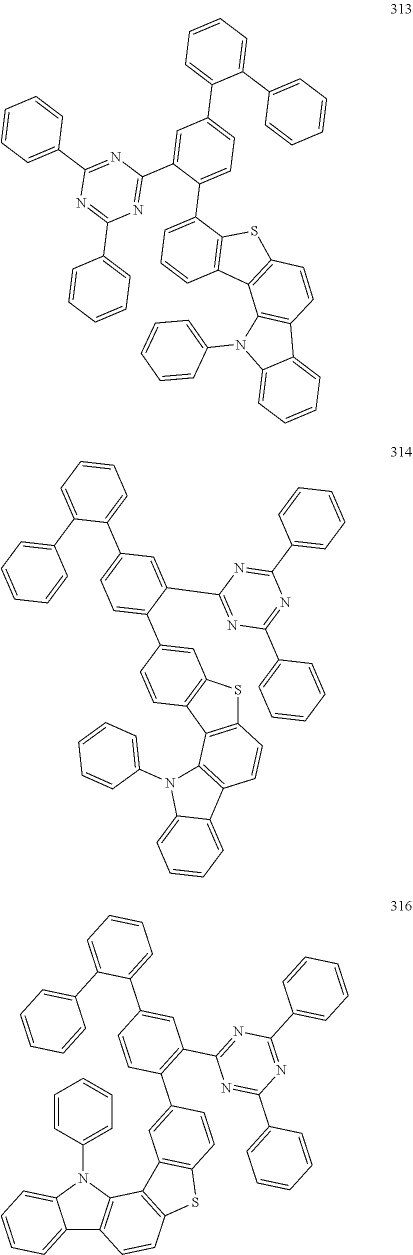 [SMILES: c1ccc(-c2nc(-c3ccccc3)nc(-c3cc(-c4ccccc4-c4ccccc4)ccc3-c3cccc4c3sc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4sc5ccc6c7ccccc7n(-c7ccccc7)c6c5c4c3)n2)cc1]

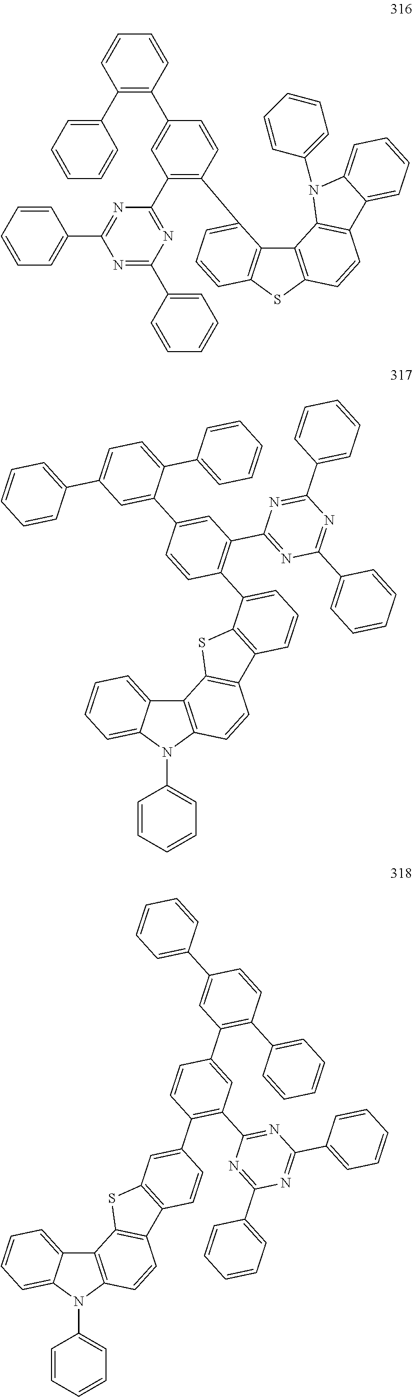 [SMILES: c1ccc(-c2nc(-c3ccccc3)nc(-c3cc(-c4ccccc4-c4ccccc4)ccc3-c3cccc4sc5ccc6c7ccccc7n(-c7ccccc7)c6c5c34)n2)cc1 ; c1ccc(-c2ccc(-c3ccccc3)c(-c3ccc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1]

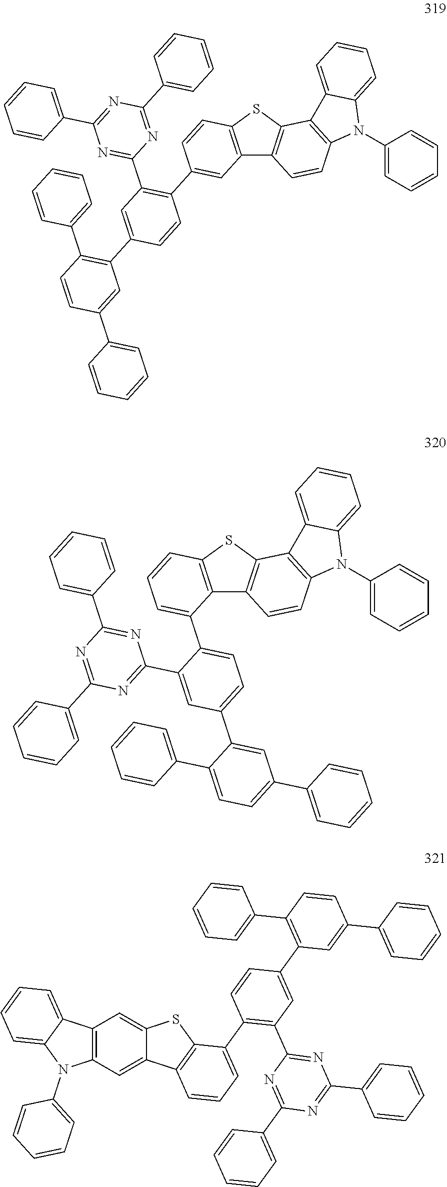 [SMILES: c1ccc(-c2ccc(-c3ccccc3)c(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4cc6c7ccccc7n(-c7ccccc7)c6cc45)c(-c4nc(-c5ccccc5)nc(-c5ccccc5)n4)c3)c2)cc1]

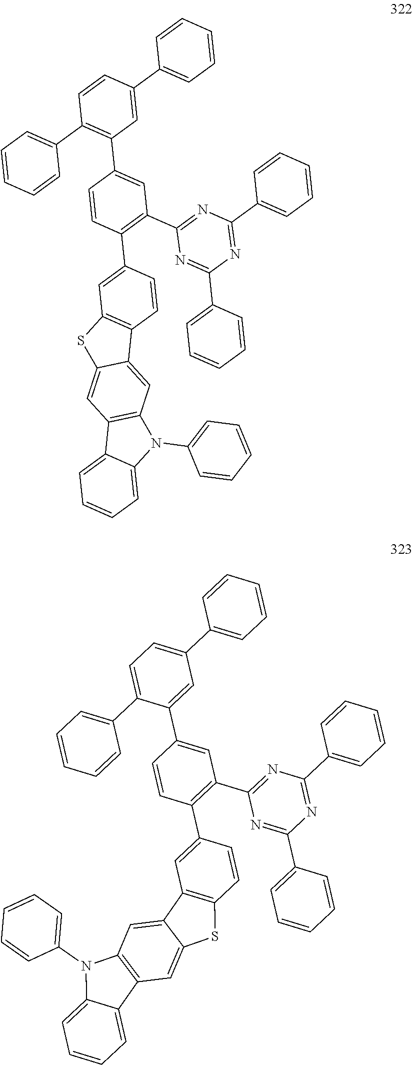 [SMILES: c1ccc(-c2ccc(-c3ccccc3)c(-c3ccc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3ccc(-c4ccc5sc6cc7c8ccccc8n(-c8ccccc8)c7cc6c5c4)c(-c4nc(-c5ccccc5)nc(-c5ccccc5)n4)c3)c2)cc1]

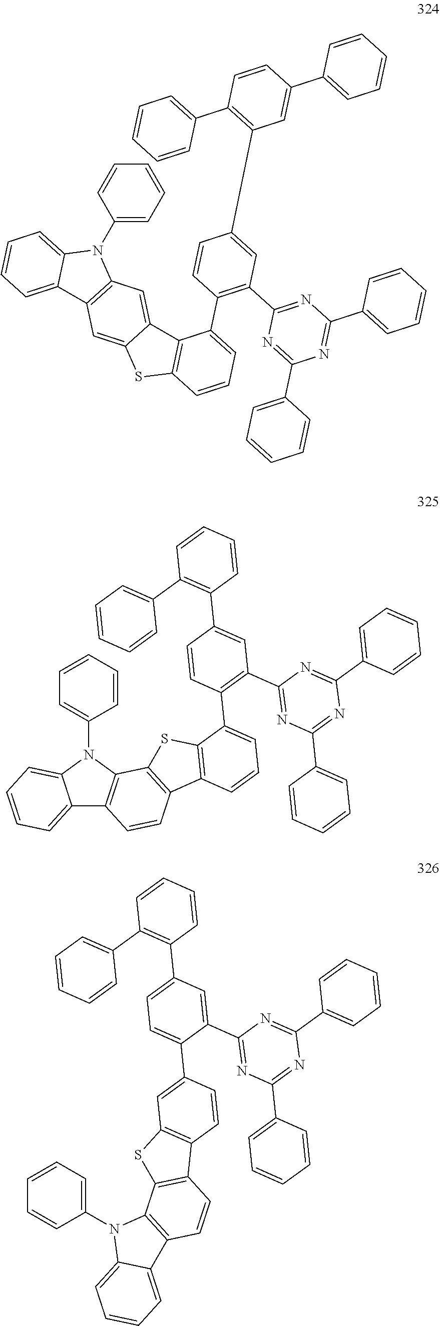 [SMILES: c1ccc(-c2ccc(-c3ccccc3)c(-c3ccc(-c4cccc5sc6cc7c8ccccc8n(-c8ccccc8)c7cc6c45)c(-c4nc(-c5ccccc5)nc(-c5ccccc5)n4)c3)c2)cc1 ; c1ccc(-c2nc(-c3ccccc3)nc(-c3cc(-c4ccccc4-c4ccccc4)ccc3-c3cccc4c3sc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4c(c3)sc3c4ccc4c5ccccc5n(-c5ccccc5)c43)n2)cc1]

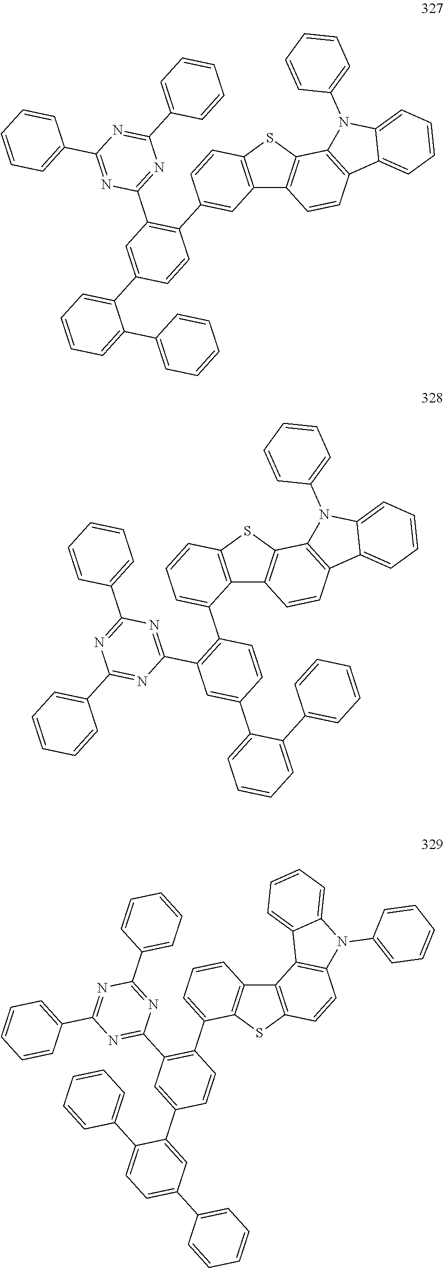 [SMILES: c1ccc(-c2nc(-c3ccccc3)nc(-c3cc(-c4ccccc4-c4ccccc4)ccc3-c3ccc4sc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)ccc3-c3cccc4sc5c(ccc6c7ccccc7n(-c7ccccc7)c65)c34)n2)cc1 ; c1ccc(-c2ccc(-c3ccccc3)c(-c3ccc(-c4cccc5c4sc4ccc6c(c7ccccc7n6-c6ccccc6)c45)c(-c4nc(-c5ccccc5)nc(-c5ccccc5)n4)c3)c2)cc1]

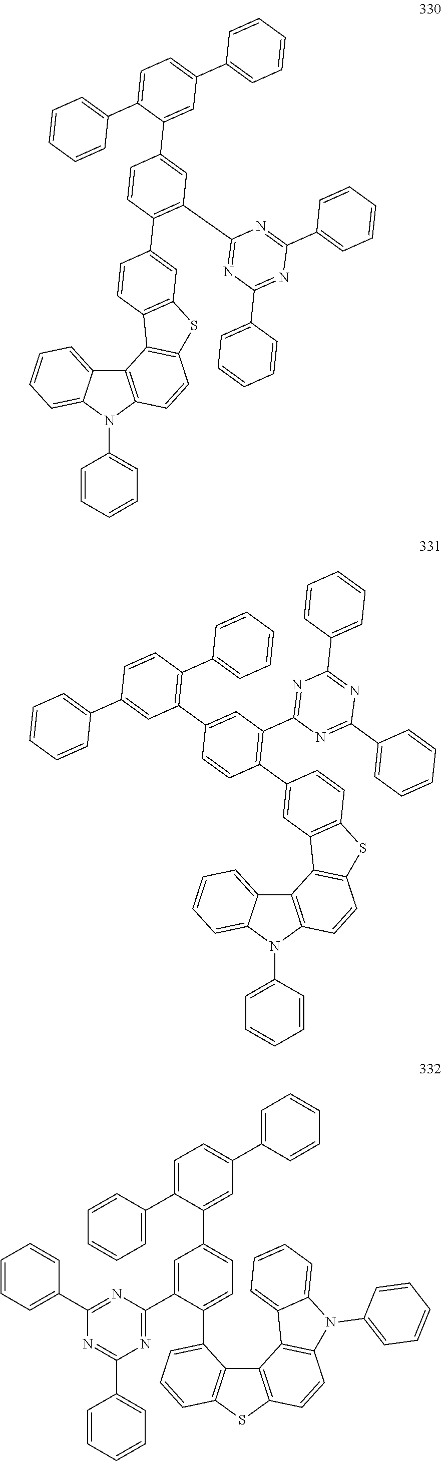 [SMILES: c1ccc(-c2ccc(-c3ccccc3)c(-c3ccc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6ccc7c(c8ccccc8n7-c7ccccc7)c6c45)c(-c4nc(-c5ccccc5)nc(-c5ccccc5)n4)c3)c2)cc1]

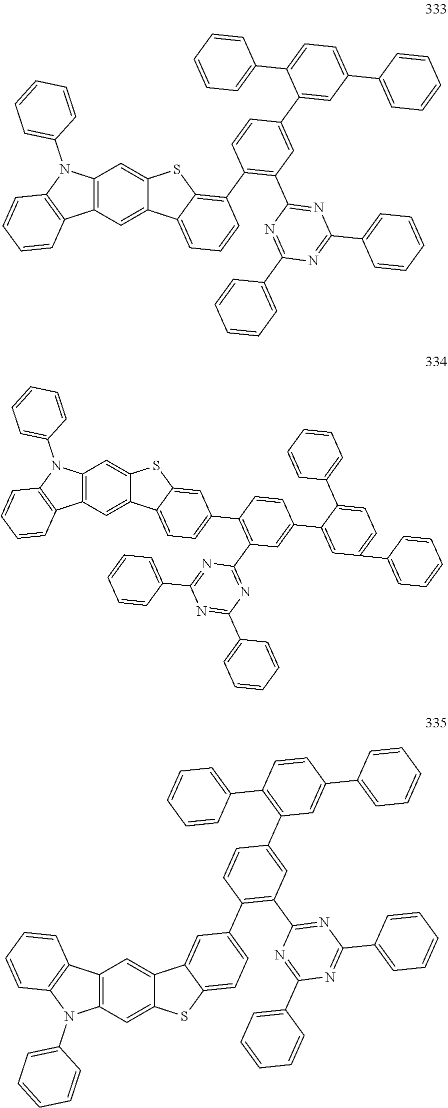 [SMILES: c1ccc(-c2ccc(-c3ccccc3)c(-c3ccc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1]

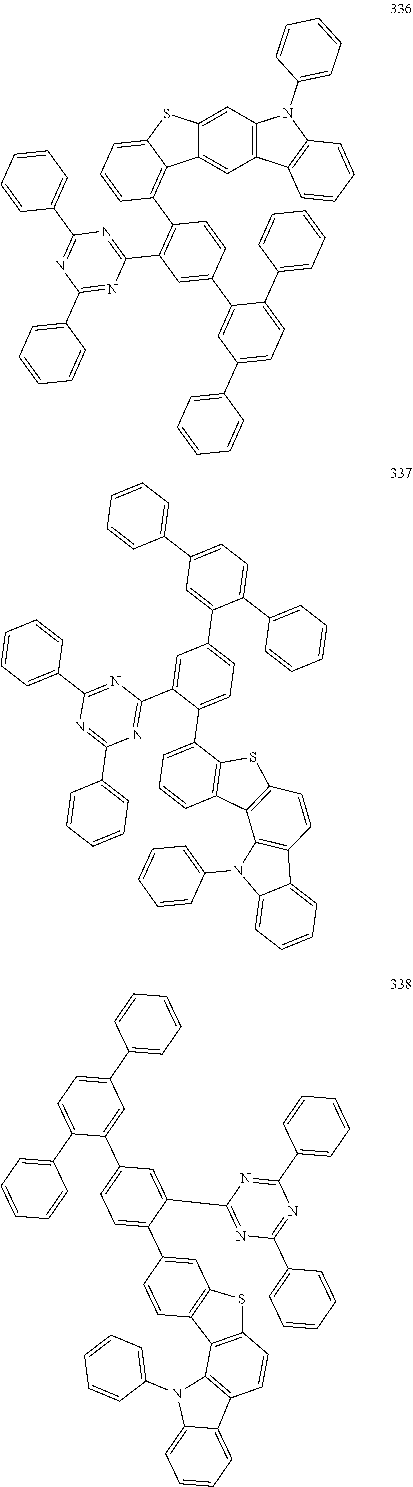 [SMILES: c1ccc(-c2ccc(-c3ccccc3)c(-c3ccc(-c4cccc5sc6cc7c(cc6c45)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3ccc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4ccc6c7ccccc7n(-c7ccccc7)c6c45)c(-c4nc(-c5ccccc5)nc(-c5ccccc5)n4)c3)c2)cc1]

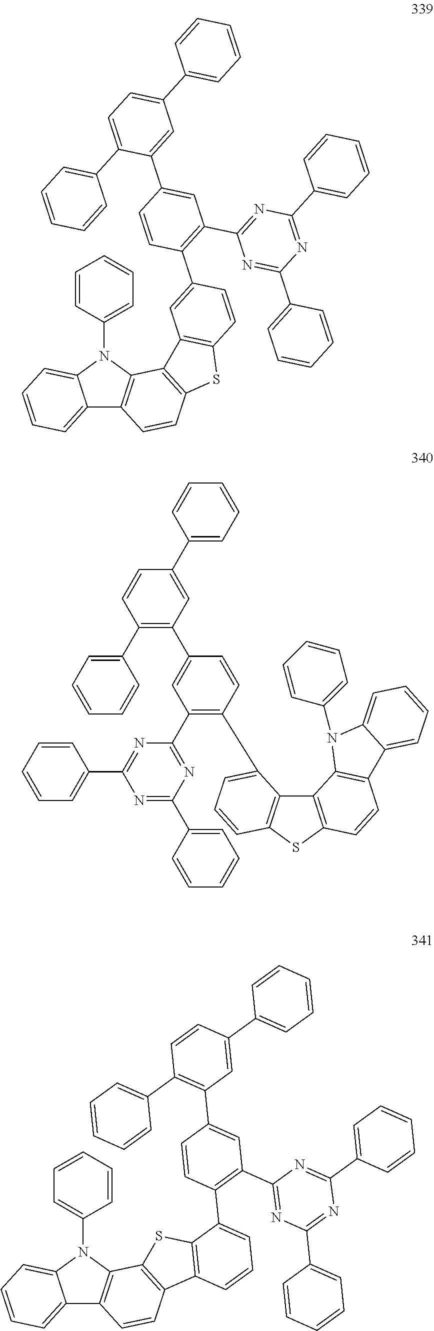 [SMILES: c1ccc(-c2ccc(-c3ccccc3)c(-c3ccc(-c4ccc5sc6ccc7c8ccccc8n(-c8ccccc8)c7c6c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4c5ccc5c6ccccc6n(-c6ccccc6)c54)c(-c4nc(-c5ccccc5)nc(-c5ccccc5)n4)c3)c2)cc1]

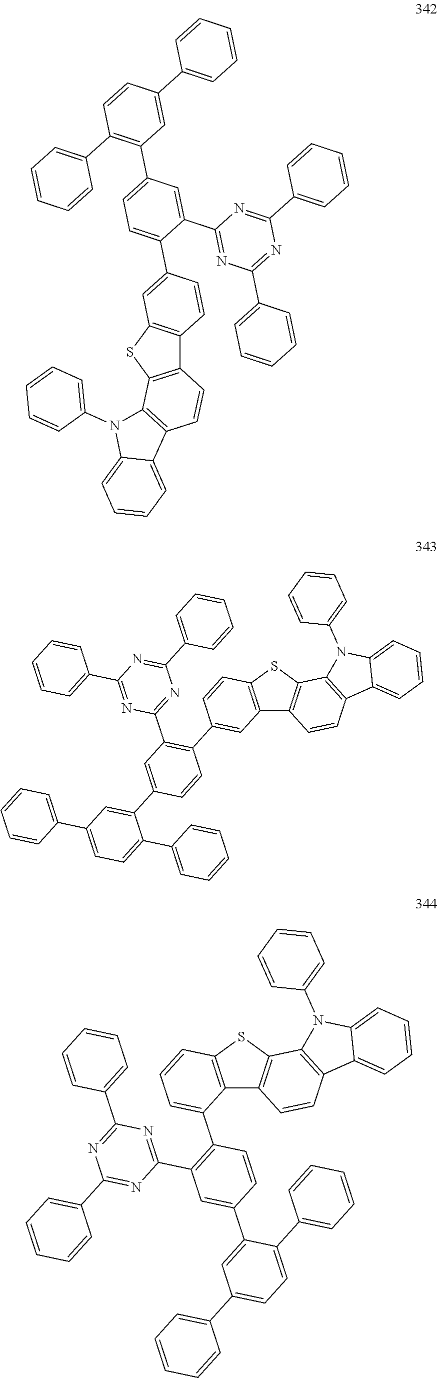 [SMILES: c1ccc(-c2ccc(-c3ccccc3)c(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6c(ccc7c8ccccc8n(-c8ccccc8)c76)c45)c(-c4nc(-c5ccccc5)nc(-c5ccccc5)n4)c3)c2)cc1]

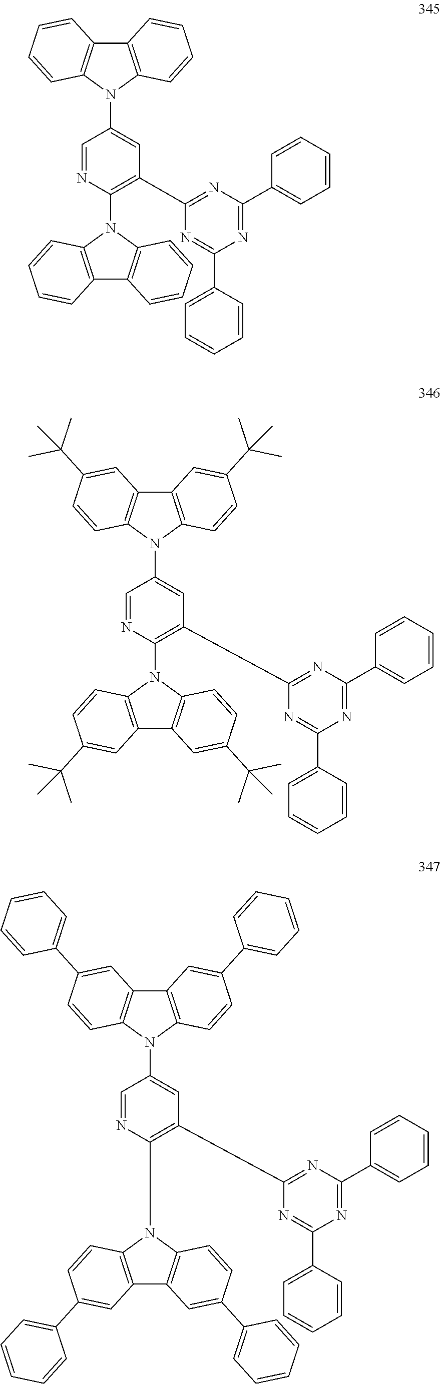 [SMILES: c1ccc(-c2nc(-c3ccccc3)nc(-c3cc(-n4c5ccccc5c5ccccc54)cnc3-n3c4ccccc4c4ccccc43)n2)cc1 ; CC(C)(C)c1ccc2c(c1)c1cc(C(C)(C)C)ccc1n2-c1cnc(-n2c3ccc(C(C)(C)C)cc3c3cc(C(C)(C)C)ccc32)c(-c2nc(-c3ccccc3)nc(-c3ccccc3)n2)c1 ; c1ccc(-c2ccc3c(c2)c2cc(-c4ccccc4)ccc2n3-c2cnc(-n3c4ccc(-c5ccccc5)cc4c4cc(-c5ccccc5)ccc43)c(-c3nc(-c4ccccc4)nc(-c4ccccc4)n3)c2)cc1]

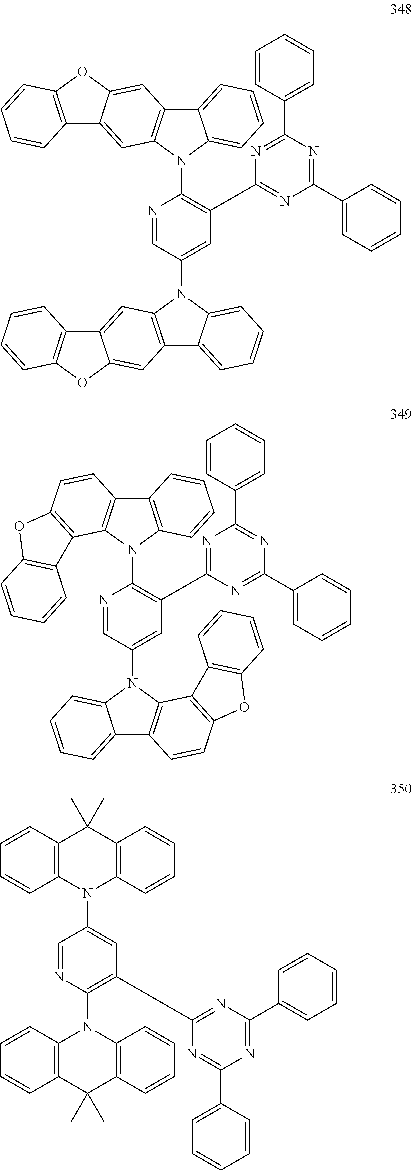

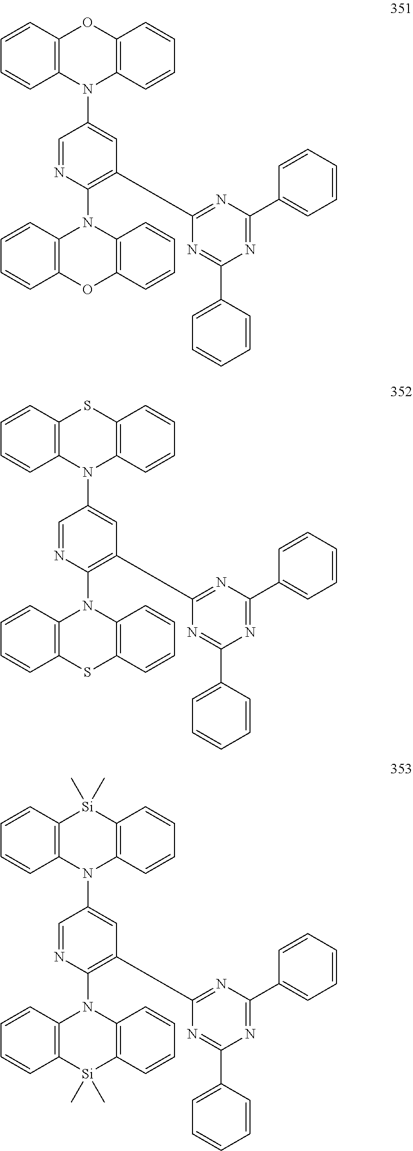 [SMILES: c1ccc(-c2nc(-c3ccccc3)nc(-c3cc(N4c5ccccc5Oc5ccccc54)cnc3N3c4ccccc4Oc4ccccc43)n2)cc1 ; c1ccc(-c2nc(-c3ccccc3)nc(-c3cc(N4c5ccccc5Sc5ccccc54)cnc3N3c4ccccc4Sc4ccccc43)n2)cc1 ; C[Si]1(C)c2ccccc2N(c2cnc(N3c4ccccc4[Si](C)(C)c4ccccc43)c(-c3nc(-c4ccccc4)nc(-c4ccccc4)n3)c2)c2ccccc21]

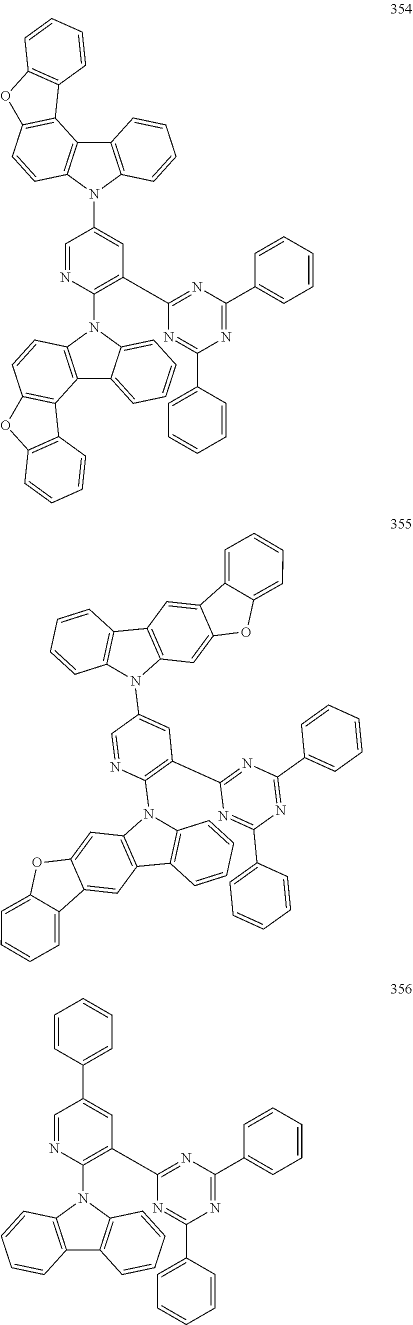 [SMILES: c1ccc(-c2nc(-c3ccccc3)nc(-c3cc(-n4c5ccccc5c5c6c(ccc54)oc4ccccc46)cnc3-n3c4ccccc4c4c5c(ccc43)oc3ccccc35)n2)cc1 ; c1ccc(-c2nc(-c3ccccc3)nc(-c3cc(-n4c5ccccc5c5cc6c(cc54)oc4ccccc46)cnc3-n3c4ccccc4c4cc5c(cc43)oc3ccccc35)n2)cc1 ; c1ccc(-c2cnc(-n3c4ccccc4c4ccccc43)c(-c3nc(-c4ccccc4)nc(-c4ccccc4)n3)c2)cc1]

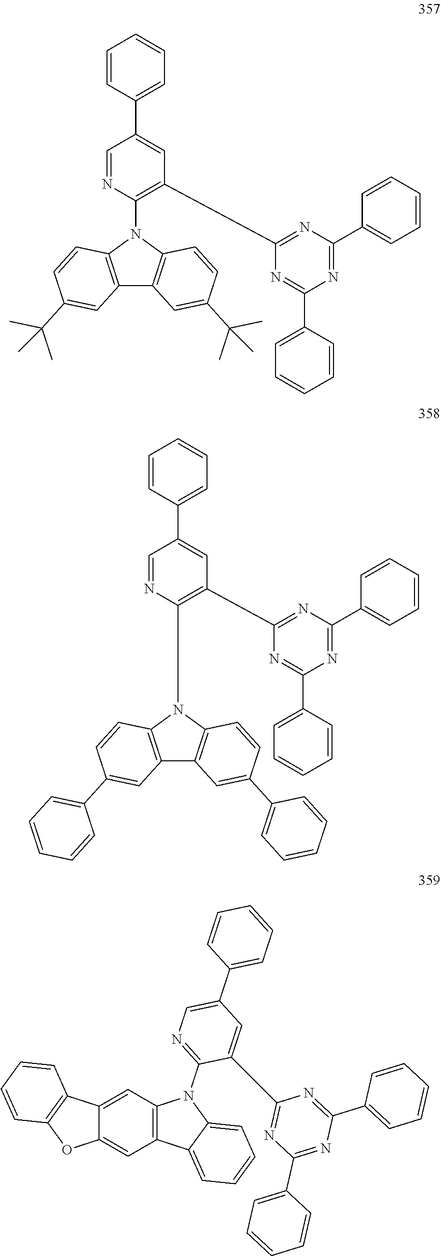 [SMILES: CC(C)(C)c1ccc2c(c1)c1cc(C(C)(C)C)ccc1n2-c1ncc(-c2ccccc2)cc1-c1nc(-c2ccccc2)nc(-c2ccccc2)n1 ; c1ccc(-c2cnc(-n3c4ccc(-c5ccccc5)cc4c4cc(-c5ccccc5)ccc43)c(-c3nc(-c4ccccc4)nc(-c4ccccc4)n3)c2)cc1 ; c1ccc(-c2cnc(-n3c4ccccc4c4cc5oc6ccccc6c5cc43)c(-c3nc(-c4ccccc4)nc(-c4ccccc4)n3)c2)cc1]

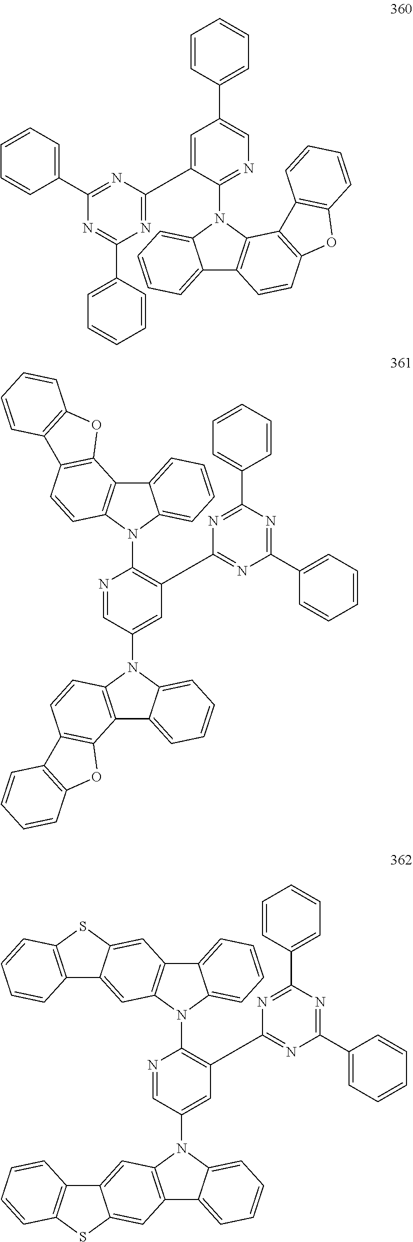 [SMILES: c1ccc(-c2cnc(-n3c4ccccc4c4ccc5oc6ccccc6c5c43)c(-c3nc(-c4ccccc4)nc(-c4ccccc4)n3)c2)cc1 ; c1ccc(-c2nc(-c3ccccc3)nc(-c3cc(-n4c5ccccc5c5c6oc7ccccc7c6ccc54)cnc3-n3c4ccccc4c4c5oc6ccccc6c5ccc43)n2)cc1 ; c1ccc(-c2nc(-c3ccccc3)nc(-c3cc(-n4c5ccccc5c5cc6sc7ccccc7c6cc54)cnc3-n3c4ccccc4c4cc5sc6ccccc6c5cc43)n2)cc1]

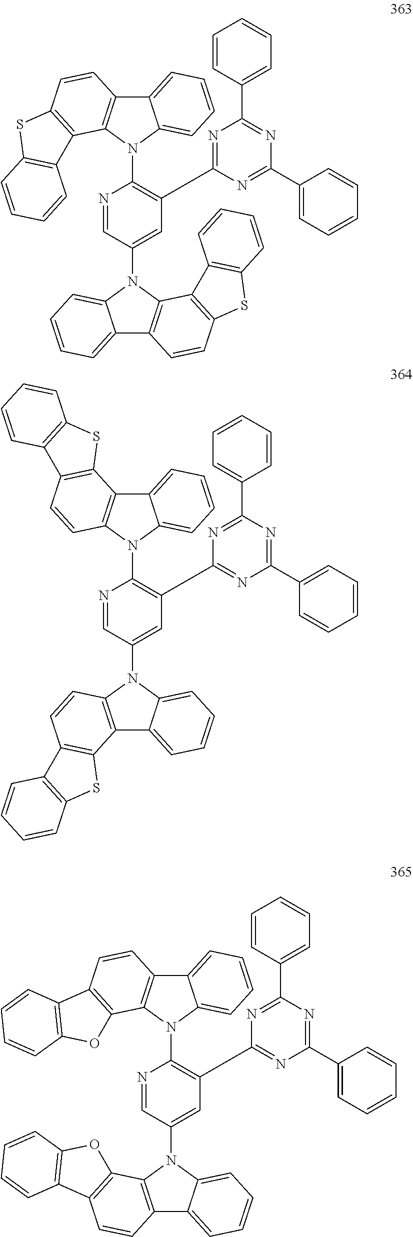

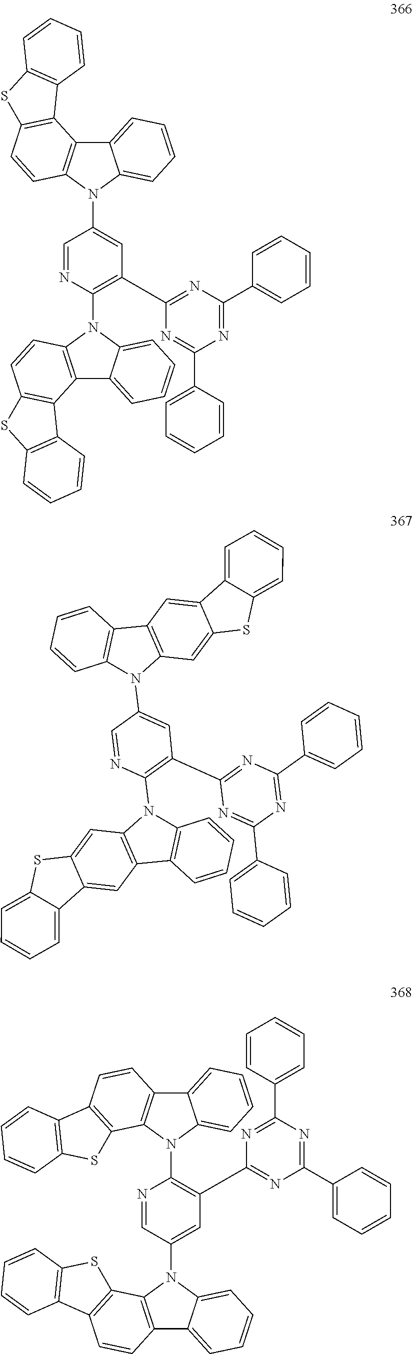 [SMILES: c1ccc(-c2nc(-c3ccccc3)nc(-c3cc(-n4c5ccccc5c5c6c(ccc54)sc4ccccc46)cnc3-n3c4ccccc4c4c5c(ccc43)sc3ccccc35)n2)cc1 ; c1ccc(-c2nc(-c3ccccc3)nc(-c3cc(-n4c5ccccc5c5cc6c(cc54)sc4ccccc46)cnc3-n3c4ccccc4c4cc5c(cc43)sc3ccccc35)n2)cc1 ; c1ccc(-c2nc(-c3ccccc3)nc(-c3cc(-n4c5ccccc5c5ccc6c7ccccc7sc6c54)cnc3-n3c4ccccc4c4ccc5c6ccccc6sc5c43)n2)cc1]

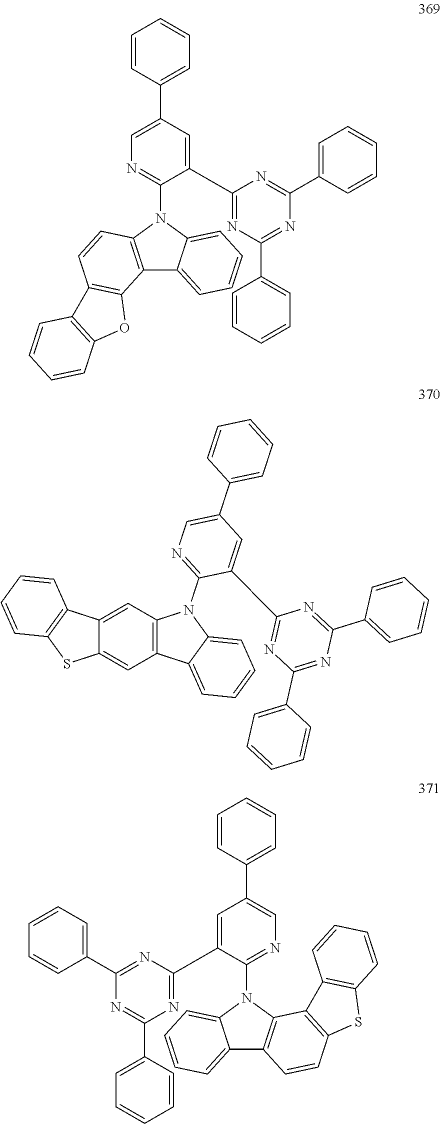

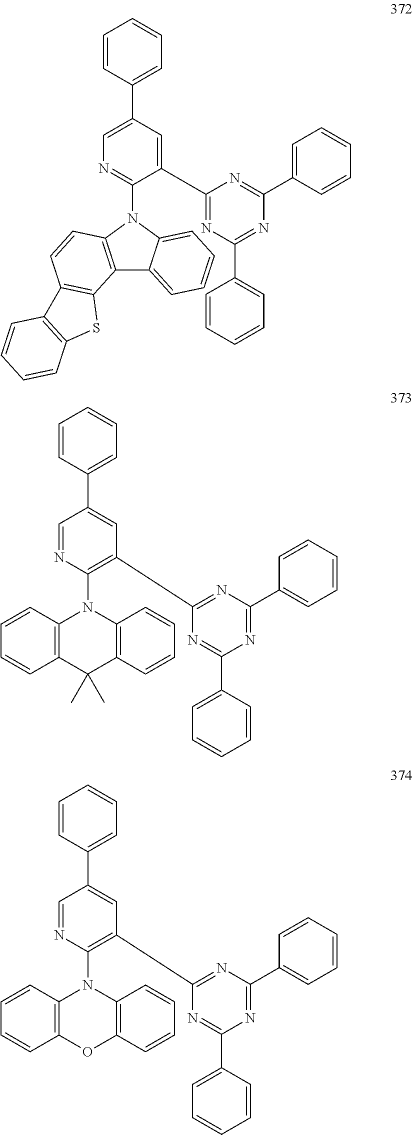 [SMILES: c1ccc(-c2cnc(-n3c4ccccc4c4c5sc6ccccc6c5ccc43)c(-c3nc(-c4ccccc4)nc(-c4ccccc4)n3)c2)cc1 ; CC1(C)c2ccccc2N(c2ncc(-c3ccccc3)cc2-c2nc(-c3ccccc3)nc(-c3ccccc3)n2)c2ccccc21 ; c1ccc(-c2cnc(N3c4ccccc4Oc4ccccc43)c(-c3nc(-c4ccccc4)nc(-c4ccccc4)n3)c2)cc1]

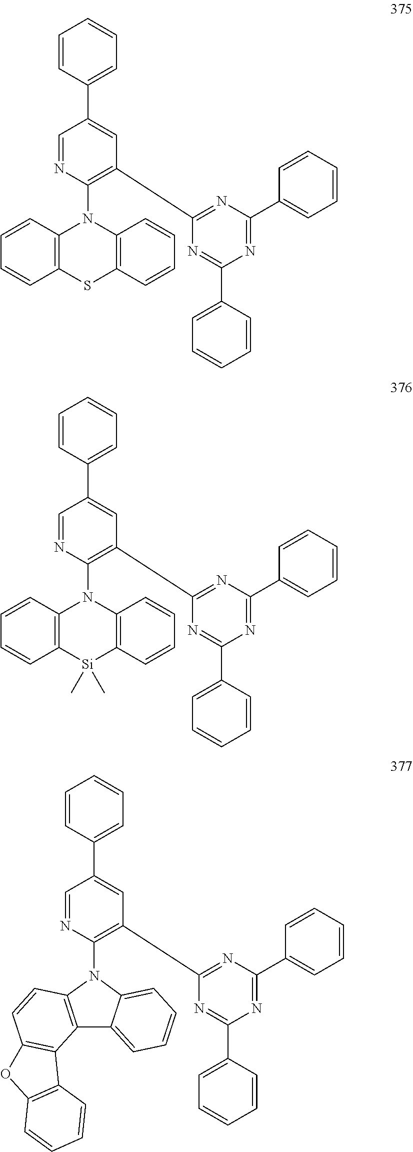 [SMILES: c1ccc(-c2cnc(N3c4ccccc4Sc4ccccc43)c(-c3nc(-c4ccccc4)nc(-c4ccccc4)n3)c2)cc1 ; C[Si]1(C)c2ccccc2N(c2ncc(-c3ccccc3)cc2-c2nc(-c3ccccc3)nc(-c3ccccc3)n2)c2ccccc21 ; c1ccc(-c2cnc(-n3c4ccccc4c4c5c(ccc43)oc3ccccc35)c(-c3nc(-c4ccccc4)nc(-c4ccccc4)n3)c2)cc1]

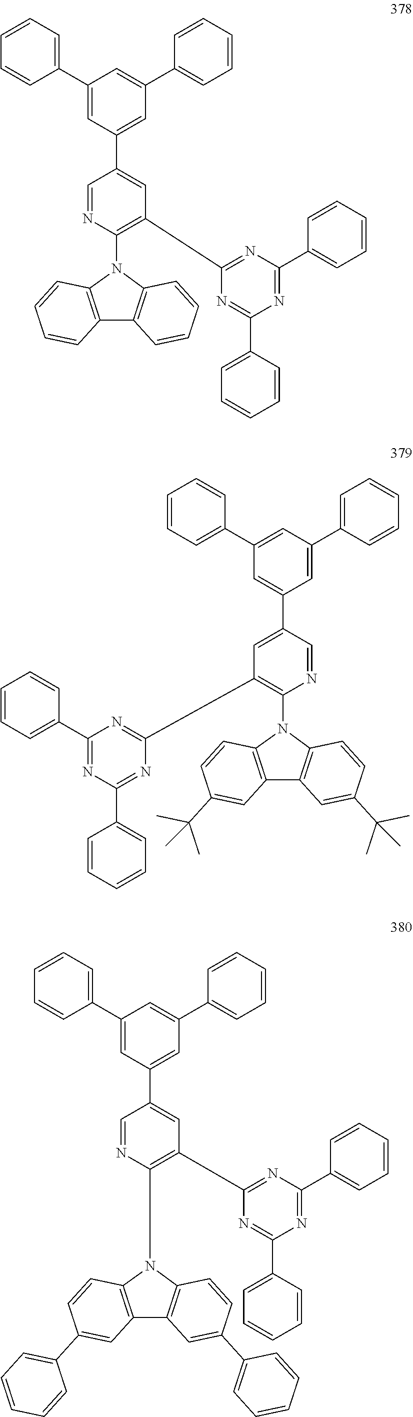 [SMILES: c1ccc(-c2cc(-c3ccccc3)cc(-c3cnc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ncc(-c2cc(-c3ccccc3)cc(-c3ccccc3)c2)cc1-c1nc(-c2ccccc2)nc(-c2ccccc2)n1 ; c1ccc(-c2cc(-c3ccccc3)cc(-c3cnc(-n4c5ccc(-c6ccccc6)cc5c5cc(-c6ccccc6)ccc54)c(-c4nc(-c5ccccc5)nc(-c5ccccc5)n4)c3)c2)cc1]

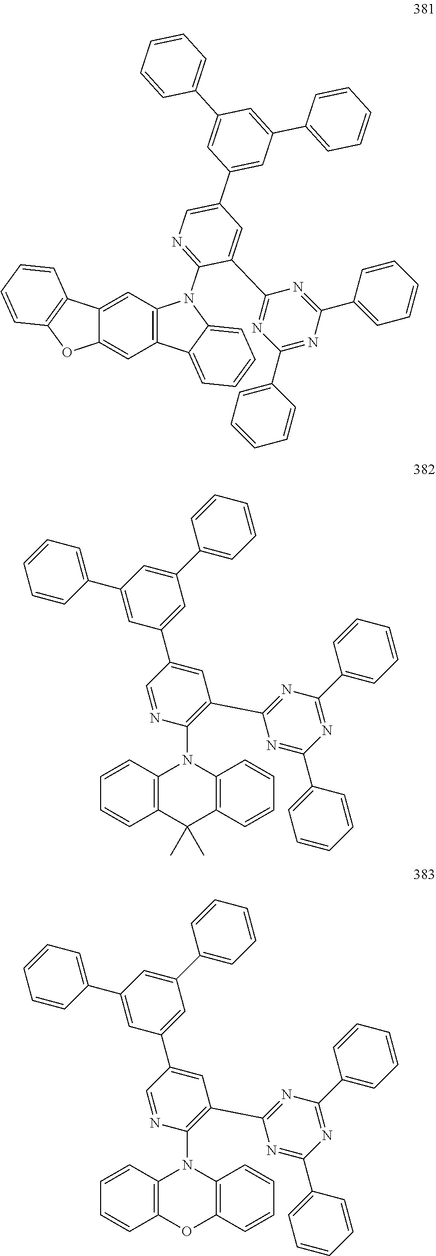

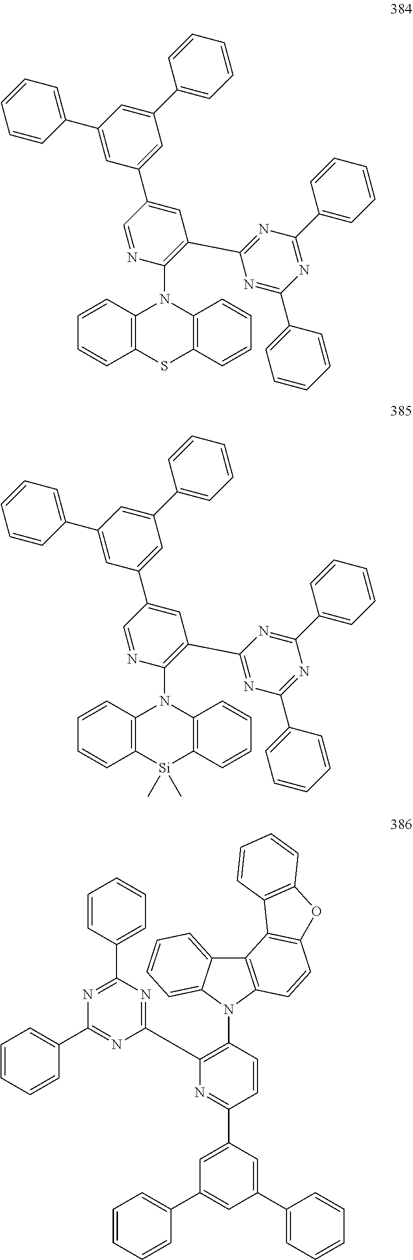 [SMILES: c1ccc(-c2cc(-c3ccccc3)cc(-c3cnc(N4c5ccccc5Sc5ccccc54)c(-c4nc(-c5ccccc5)nc(-c5ccccc5)n4)c3)c2)cc1 ; C[Si]1(C)c2ccccc2N(c2ncc(-c3cc(-c4ccccc4)cc(-c4ccccc4)c3)cc2-c2nc(-c3ccccc3)nc(-c3ccccc3)n2)c2ccccc21 ; c1ccc(-c2cc(-c3ccccc3)cc(-c3ccc(-n4c5ccccc5c5c6c(ccc54)oc4ccccc46)c(-c4nc(-c5ccccc5)nc(-c5ccccc5)n4)n3)c2)cc1]

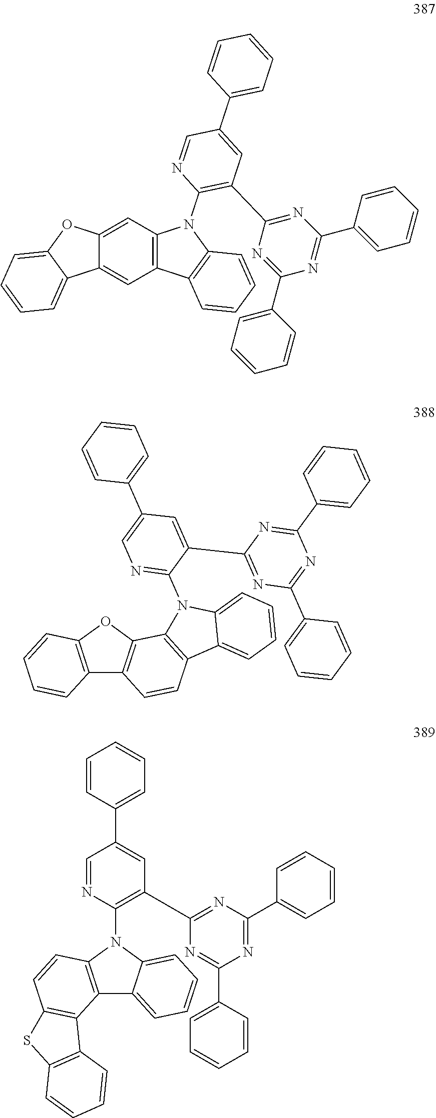 [SMILES: c1ccc(-c2cnc(-n3c4ccccc4c4cc5c(cc43)oc3ccccc35)c(-c3nc(-c4ccccc4)nc(-c4ccccc4)n3)c2)cc1 ; c1ccc(-c2cnc(-n3c4ccccc4c4ccc5c6ccccc6oc5c43)c(-c3nc(-c4ccccc4)nc(-c4ccccc4)n3)c2)cc1 ; c1ccc(-c2cnc(-n3c4ccccc4c4c5c(ccc43)sc3ccccc35)c(-c3nc(-c4ccccc4)nc(-c4ccccc4)n3)c2)cc1]

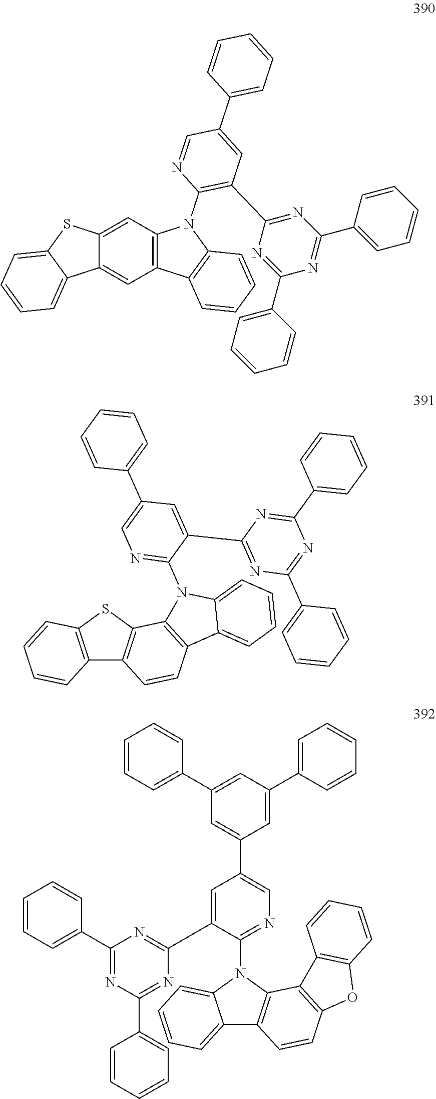

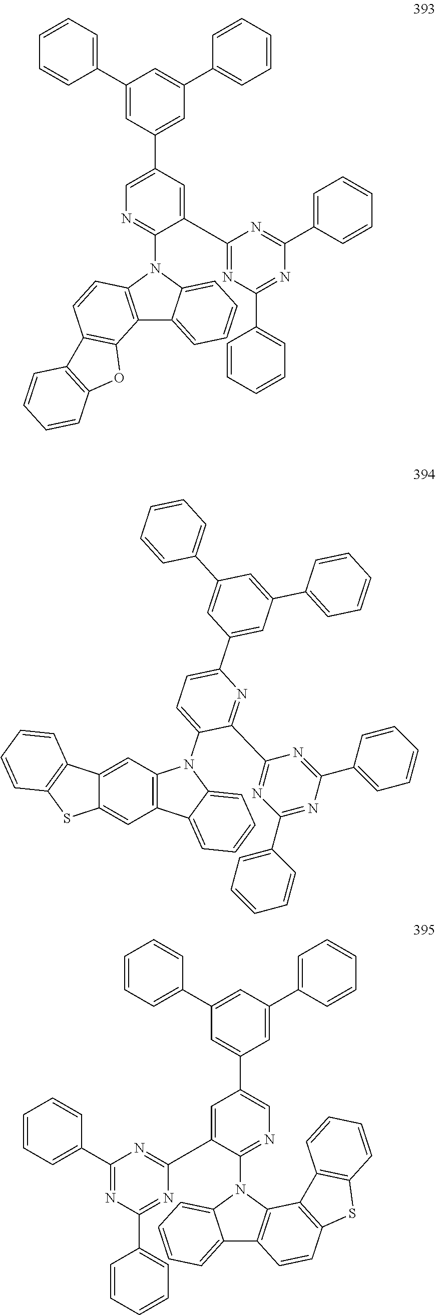 [SMILES: c1ccc(-c2cc(-c3ccccc3)cc(-c3cnc(-n4c5ccccc5c5c6oc7ccccc7c6ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6sc7ccccc7c6cc54)c(-c4nc(-c5ccccc5)nc(-c5ccccc5)n4)n3)c2)cc1 ; c1ccc(-c2cc(-c3ccccc3)cc(-c3cnc(-n4c5ccccc5c5ccc6sc7ccccc7c6c54)c(-c4nc(-c5ccccc5)nc(-c5ccccc5)n4)c3)c2)cc1]

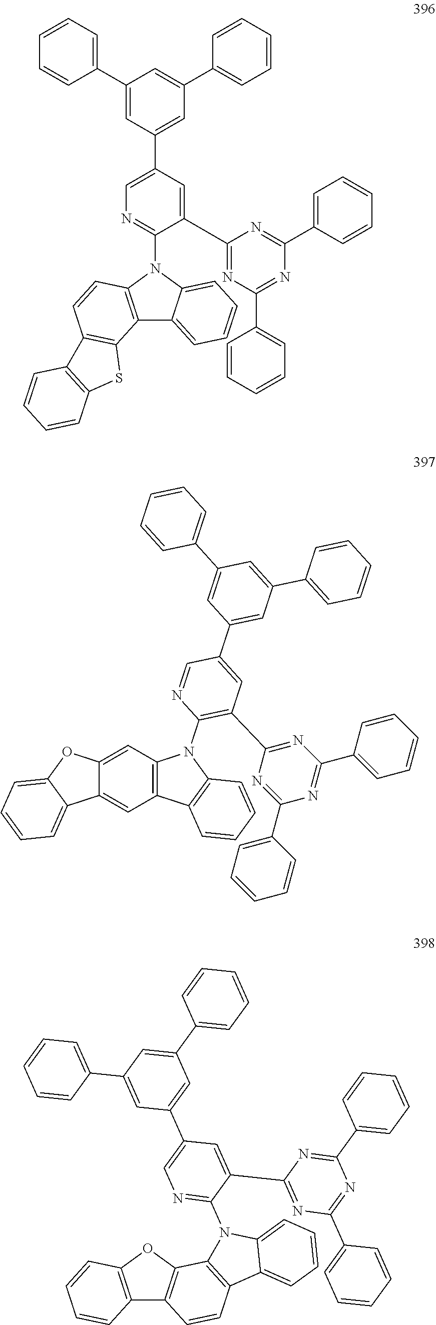 [SMILES: c1ccc(-c2cc(-c3ccccc3)cc(-c3cnc(-n4c5ccccc5c5c6sc7ccccc7c6ccc54)c(-c4nc(-c5ccccc5)nc(-c5ccccc5)n4)c3)c2)cc1 ; c1ccc(-c2cc(-c3ccccc3)cc(-c3cnc(-n4c5ccccc5c5cc6c(cc54)oc4ccccc46)c(-c4nc(-c5ccccc5)nc(-c5ccccc5)n4)c3)c2)cc1 ; c1ccc(-c2cc(-c3ccccc3)cc(-c3cnc(-n4c5ccccc5c5ccc6c7ccccc7oc6c54)c(-c4nc(-c5ccccc5)nc(-c5ccccc5)n4)c3)c2)cc1]

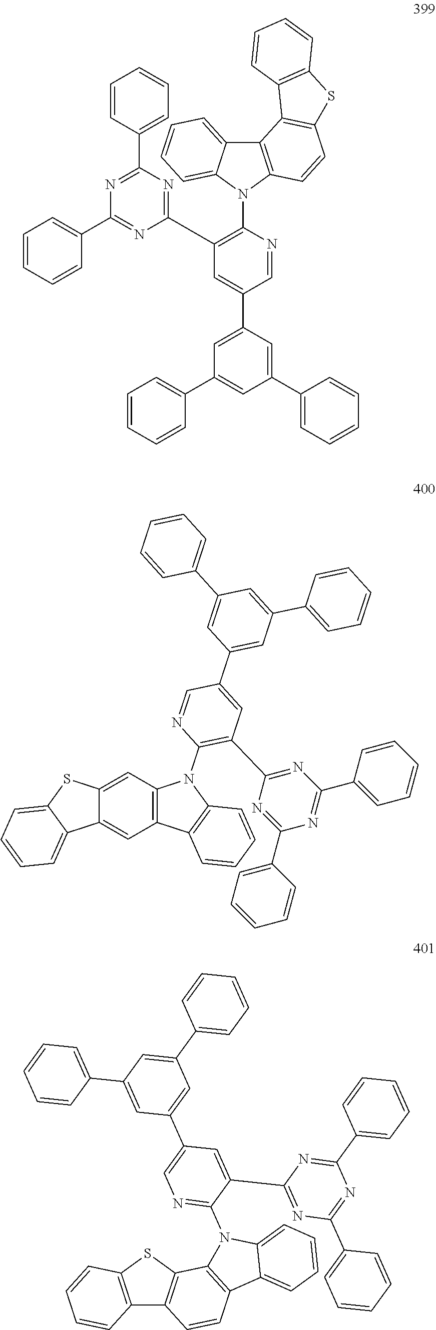 [SMILES: c1ccc(-c2cc(-c3ccccc3)cc(-c3cnc(-n4c5ccccc5c5c6c(ccc54)sc4ccccc46)c(-c4nc(-c5ccccc5)nc(-c5ccccc5)n4)c3)c2)cc1 ; c1ccc(-c2cc(-c3ccccc3)cc(-c3cnc(-n4c5ccccc5c5cc6c(cc54)sc4ccccc46)c(-c4nc(-c5ccccc5)nc(-c5ccccc5)n4)c3)c2)cc1 ; c1ccc(-c2cc(-c3ccccc3)cc(-c3cnc(-n4c5ccccc5c5ccc6c7ccccc7sc6c54)c(-c4nc(-c5ccccc5)nc(-c5ccccc5)n4)c3)c2)cc1]

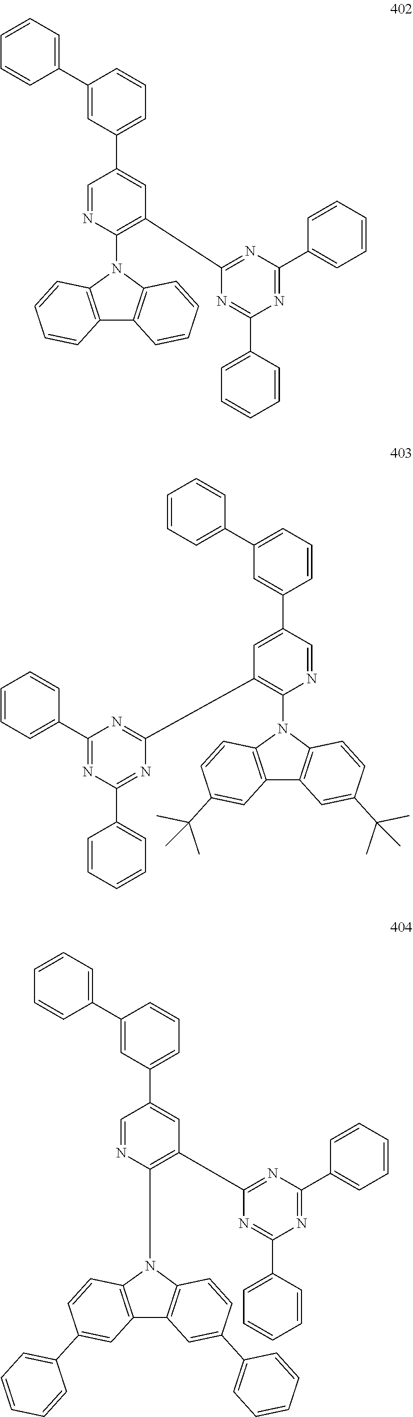 [SMILES: c1ccc(-c2cccc(-c3cnc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ncc(-c2cccc(-c3ccccc3)c2)cc1-c1nc(-c2ccccc2)nc(-c2ccccc2)n1 ; c1ccc(-c2cccc(-c3cnc(-n4c5ccc(-c6ccccc6)cc5c5cc(-c6ccccc6)ccc54)c(-c4nc(-c5ccccc5)nc(-c5ccccc5)n4)c3)c2)cc1]

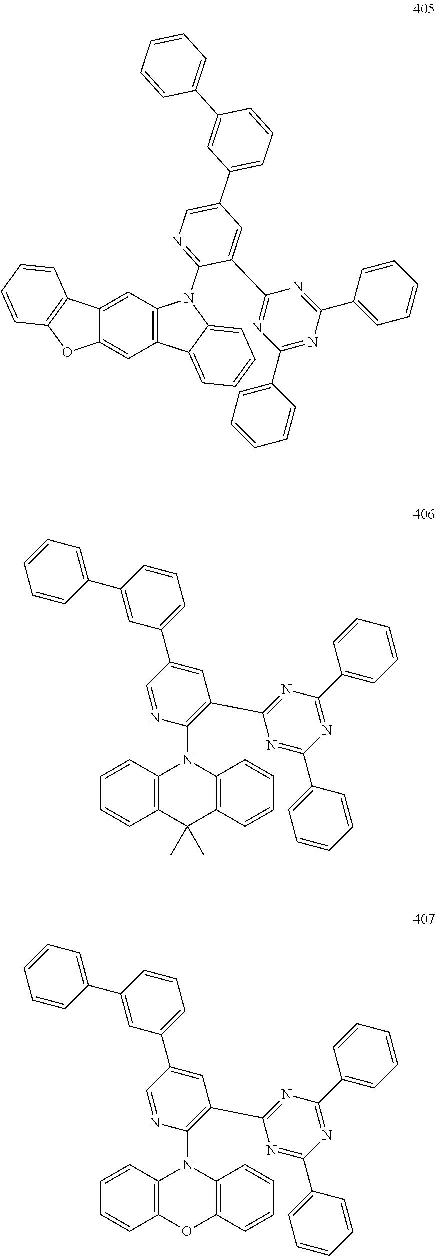 [SMILES: c1ccc(-c2cccc(-c3cnc(-n4c5ccccc5c5cc6oc7ccccc7c6cc54)c(-c4nc(-c5ccccc5)nc(-c5ccccc5)n4)c3)c2)cc1 ; CC1(C)c2ccccc2N(c2ncc(-c3cccc(-c4ccccc4)c3)cc2-c2nc(-c3ccccc3)nc(-c3ccccc3)n2)c2ccccc21 ; c1ccc(-c2cccc(-c3cnc(N4c5ccccc5Oc5ccccc54)c(-c4nc(-c5ccccc5)nc(-c5ccccc5)n4)c3)c2)cc1]

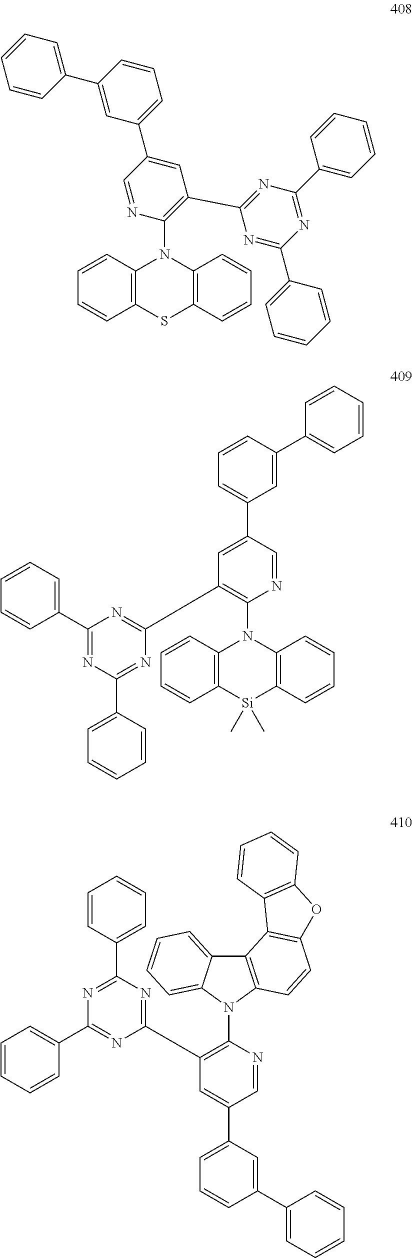 [SMILES: c1ccc(-c2cccc(-c3cnc(N4c5ccccc5Sc5ccccc54)c(-c4nc(-c5ccccc5)nc(-c5ccccc5)n4)c3)c2)cc1 ; C[Si]1(C)c2ccccc2N(c2ncc(-c3cccc(-c4ccccc4)c3)cc2-c2nc(-c3ccccc3)nc(-c3ccccc3)n2)c2ccccc21 ; c1ccc(-c2cccc(-c3cnc(-n4c5ccccc5c5c6c(ccc54)oc4ccccc46)c(-c4nc(-c5ccccc5)nc(-c5ccccc5)n4)c3)c2)cc1]

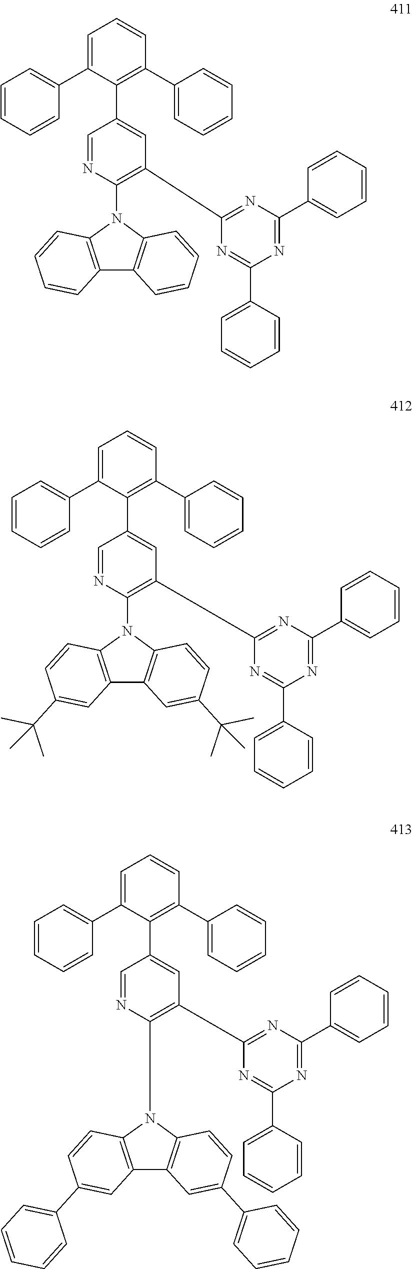 [SMILES: c1ccc(-c2nc(-c3ccccc3)nc(-c3cc(-c4c(-c5ccccc5)cccc4-c4ccccc4)cnc3-n3c4ccccc4c4ccccc43)n2)cc1 ; CC(C)(C)c1ccc2c(c1)c1cc(C(C)(C)C)ccc1n2-c1ncc(-c2c(-c3ccccc3)cccc2-c2ccccc2)cc1-c1nc(-c2ccccc2)nc(-c2ccccc2)n1 ; c1ccc(-c2ccc3c(c2)c2cc(-c4ccccc4)ccc2n3-c2ncc(-c3c(-c4ccccc4)cccc3-c3ccccc3)cc2-c2nc(-c3ccccc3)nc(-c3ccccc3)n2)cc1]

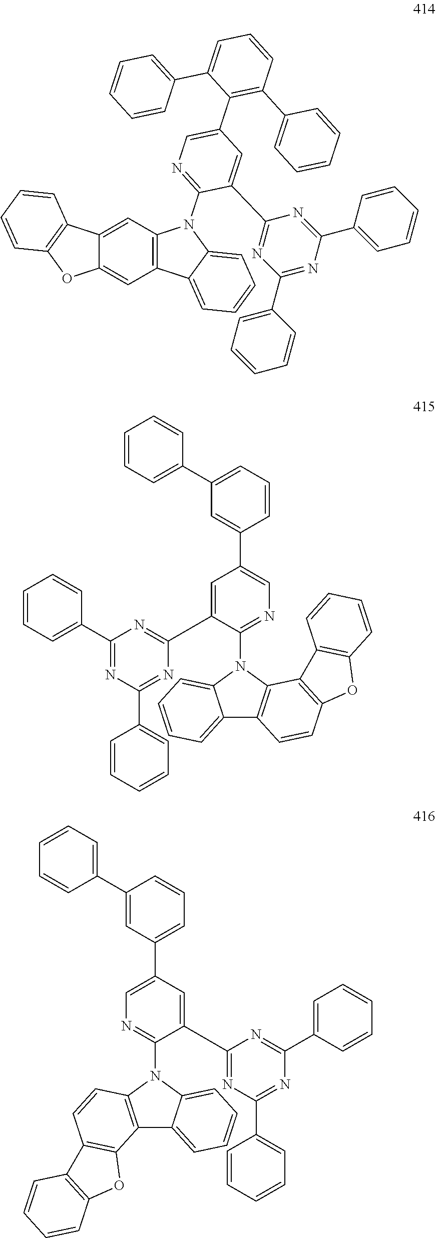 [SMILES: c1ccc(-c2nc(-c3ccccc3)nc(-c3cc(-c4c(-c5ccccc5)cccc4-c4ccccc4)cnc3-n3c4ccccc4c4cc5oc6ccccc6c5cc43)n2)cc1 ; c1ccc(-c2cccc(-c3cnc(-n4c5ccccc5c5ccc6oc7ccccc7c6c54)c(-c4nc(-c5ccccc5)nc(-c5ccccc5)n4)c3)c2)cc1 ; c1ccc(-c2cccc(-c3cnc(-n4c5ccccc5c5c6oc7ccccc7c6ccc54)c(-c4nc(-c5ccccc5)nc(-c5ccccc5)n4)c3)c2)cc1]

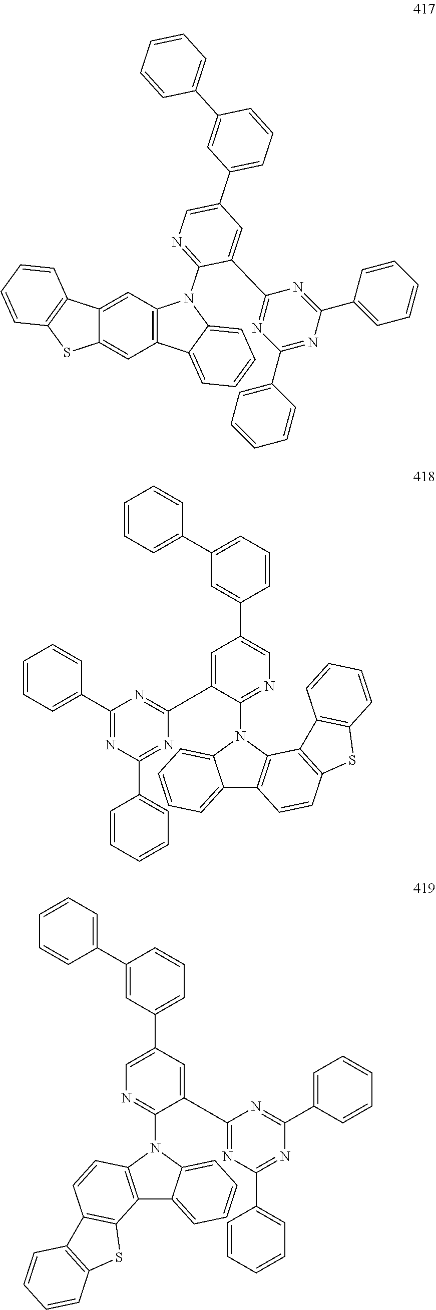 [SMILES: c1ccc(-c2cccc(-c3cnc(-n4c5ccccc5c5cc6sc7ccccc7c6cc54)c(-c4nc(-c5ccccc5)nc(-c5ccccc5)n4)c3)c2)cc1 ; c1ccc(-c2cccc(-c3cnc(-n4c5ccccc5c5ccc6sc7ccccc7c6c54)c(-c4nc(-c5ccccc5)nc(-c5ccccc5)n4)c3)c2)cc1 ; c1ccc(-c2cccc(-c3cnc(-n4c5ccccc5c5c6sc7ccccc7c6ccc54)c(-c4nc(-c5ccccc5)nc(-c5ccccc5)n4)c3)c2)cc1]

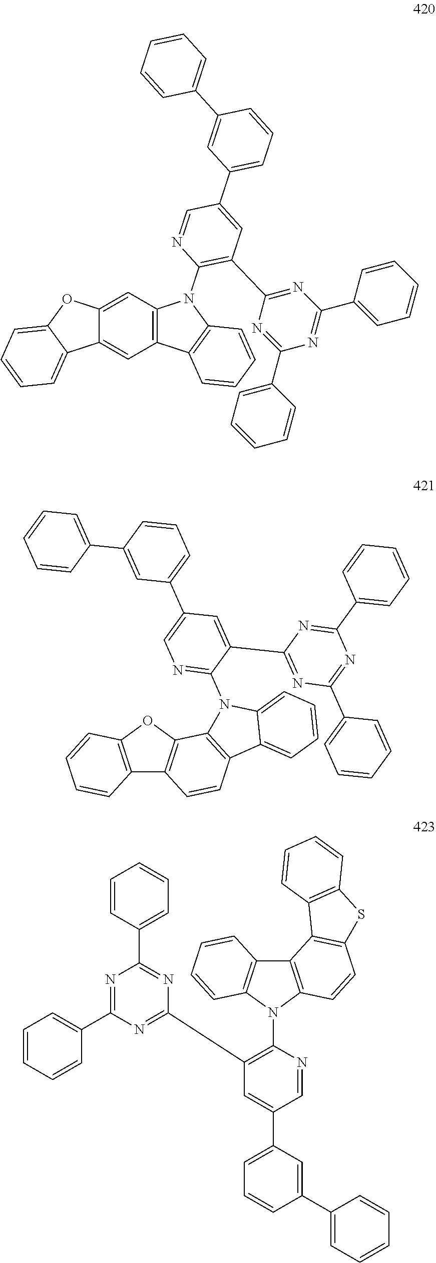 [SMILES: c1ccc(-c2cccc(-c3cnc(-n4c5ccccc5c5cc6c(cc54)oc4ccccc46)c(-c4nc(-c5ccccc5)nc(-c5ccccc5)n4)c3)c2)cc1 ; c1ccc(-c2cccc(-c3cnc(-n4c5ccccc5c5ccc6c7ccccc7oc6c54)c(-c4nc(-c5ccccc5)nc(-c5ccccc5)n4)c3)c2)cc1 ; c1ccc(-c2cccc(-c3cnc(-n4c5ccccc5c5c6c(ccc54)sc4ccccc46)c(-c4nc(-c5ccccc5)nc(-c5ccccc5)n4)c3)c2)cc1]

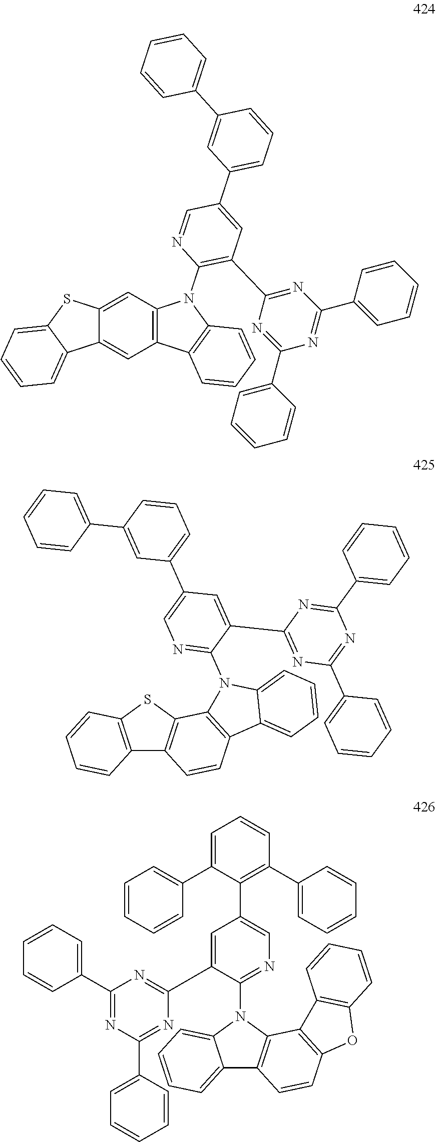 [SMILES: c1ccc(-c2cccc(-c3cnc(-n4c5ccccc5c5cc6c(cc54)sc4ccccc46)c(-c4nc(-c5ccccc5)nc(-c5ccccc5)n4)c3)c2)cc1 ; c1ccc(-c2cccc(-c3cnc(-n4c5ccccc5c5ccc6c7ccccc7sc6c54)c(-c4nc(-c5ccccc5)nc(-c5ccccc5)n4)c3)c2)cc1 ; c1ccc(-c2nc(-c3ccccc3)nc(-c3cc(-c4c(-c5ccccc5)cccc4-c4ccccc4)cnc3-n3c4ccccc4c4ccc5oc6ccccc6c5c43)n2)cc1]

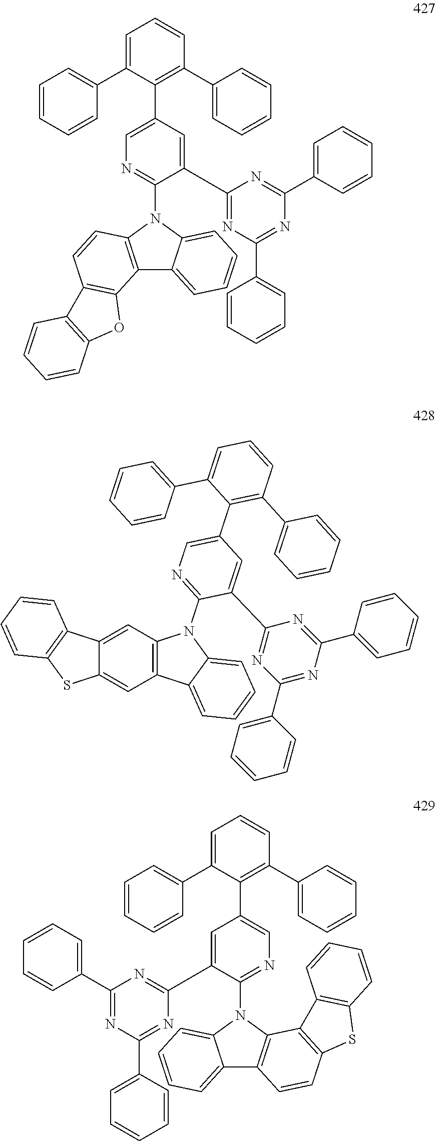

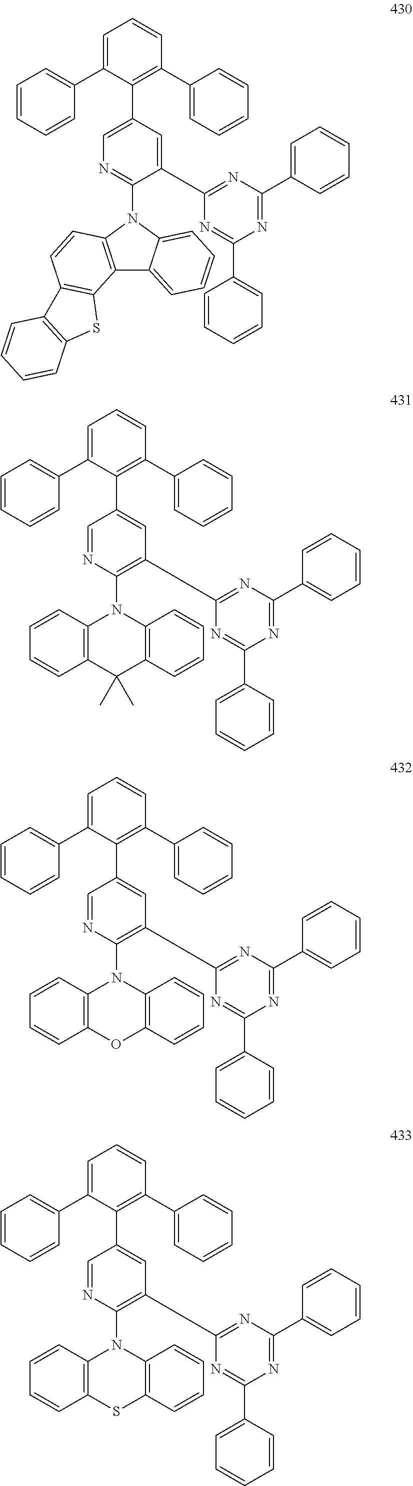 [SMILES: c1ccc(-c2nc(-c3ccccc3)nc(-c3cc(-c4c(-c5ccccc5)cccc4-c4ccccc4)cnc3-n3c4ccccc4c4c5sc6ccccc6c5ccc43)n2)cc1 ; CC1(C)c2ccccc2N(c2ncc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)cnc3N3c4ccccc4Oc4ccccc43)n2)cc1 ; c1ccc(-c2nc(-c3ccccc3)nc(-c3cc(-c4c(-c5ccccc5)cccc4-c4ccccc4)cnc3N3c4ccccc4Sc4ccccc43)n2)cc1]

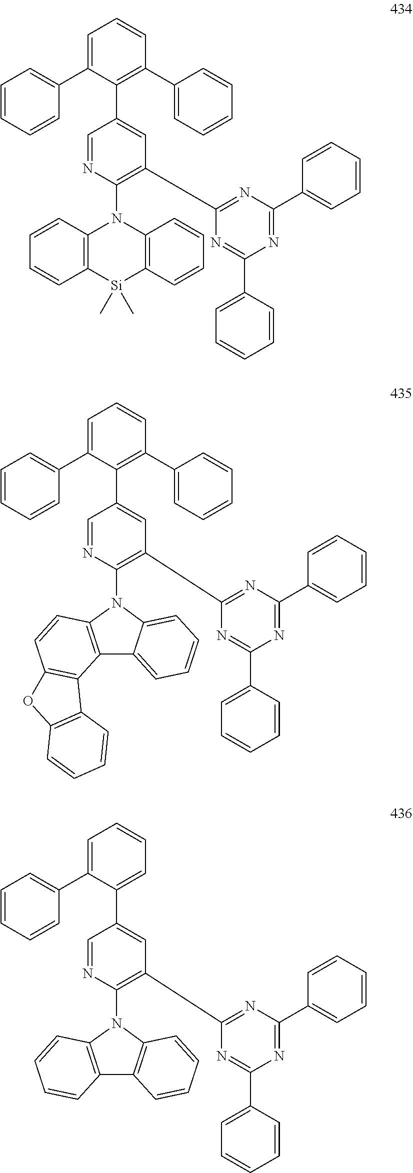 [SMILES: C[Si]1(C)c2ccccc2N(c2ncc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)cnc3-n3c4ccccc4c4c5c(ccc43)oc3ccccc35)n2)cc1 ; c1ccc(-c2nc(-c3ccccc3)nc(-c3cc(-c4ccccc4-c4ccccc4)cnc3-n3c4ccccc4c4ccccc43)n2)cc1]

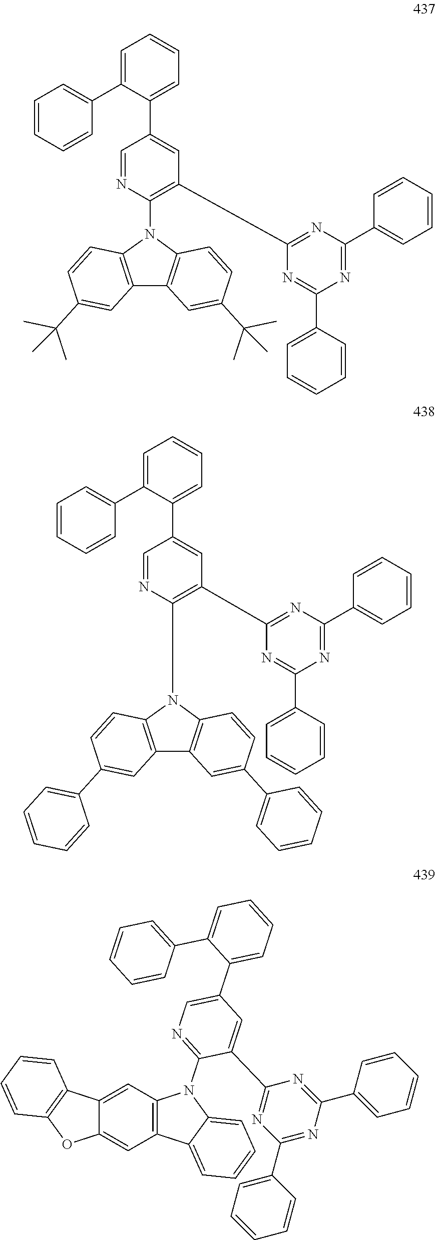 [SMILES: CC(C)(C)c1ccc2c(c1)c1cc(C(C)(C)C)ccc1n2-c1ncc(-c2ccccc2-c2ccccc2)cc1-c1nc(-c2ccccc2)nc(-c2ccccc2)n1 ; c1ccc(-c2ccc3c(c2)c2cc(-c4ccccc4)ccc2n3-c2ncc(-c3ccccc3-c3ccccc3)cc2-c2nc(-c3ccccc3)nc(-c3ccccc3)n2)cc1 ; c1ccc(-c2nc(-c3ccccc3)nc(-c3cc(-c4ccccc4-c4ccccc4)cnc3-n3c4ccccc4c4cc5oc6ccccc6c5cc43)n2)cc1]

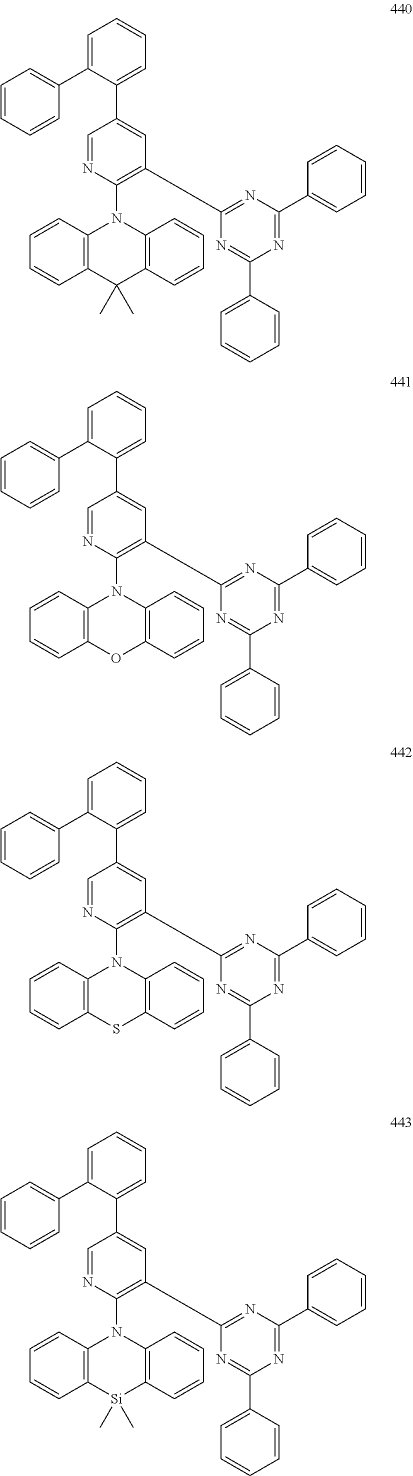 [SMILES: CC1(C)c2ccccc2N(c2ncc(-c3ccccc3-c3ccccc3)cc2-c2nc(-c3ccccc3)nc(-c3ccccc3)n2)c2ccccc21 ; c1ccc(-c2nc(-c3ccccc3)nc(-c3cc(-c4ccccc4-c4ccccc4)cnc3N3c4ccccc4Oc4ccccc43)n2)cc1 ; c1ccc(-c2nc(-c3ccccc3)nc(-c3cc(-c4ccccc4-c4ccccc4)cnc3N3c4ccccc4Sc4ccccc43)n2)cc1 ; C[Si]1(C)c2ccccc2N(c2ncc(-c3ccccc3-c3ccccc3)cc2-c2nc(-c3ccccc3)nc(-c3ccccc3)n2)c2ccccc21]

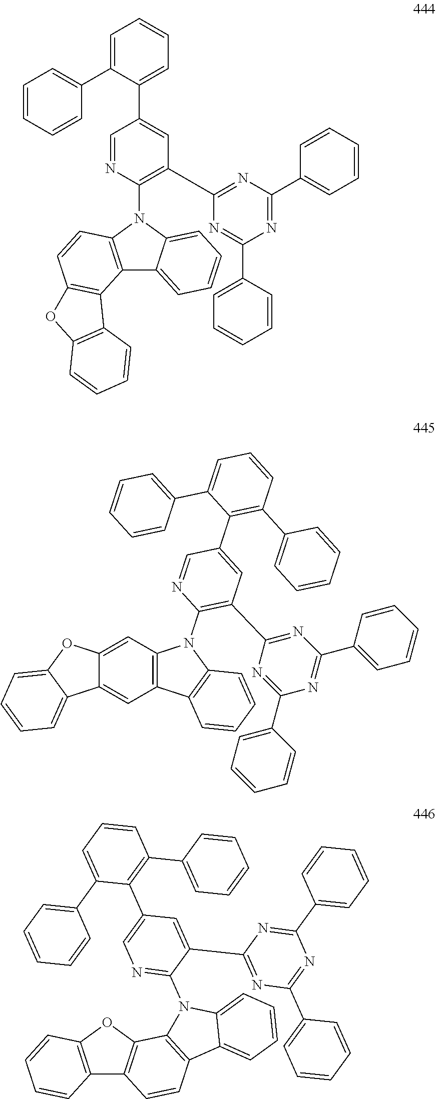 [SMILES: c1ccc(-c2nc(-c3ccccc3)nc(-c3cc(-c4ccccc4-c4ccccc4)cnc3-n3c4ccccc4c4c5c(ccc43)oc3ccccc35)n2)cc1 ; c1ccc(-c2nc(-c3ccccc3)nc(-c3cc(-c4c(-c5ccccc5)cccc4-c4ccccc4)cnc3-n3c4ccccc4c4cc5c(cc43)oc3ccccc35)n2)cc1 ; c1ccc(-c2nc(-c3ccccc3)nc(-c3cc(-c4c(-c5ccccc5)cccc4-c4ccccc4)cnc3-n3c4ccccc4c4ccc5c6ccccc6oc5c43)n2)cc1]

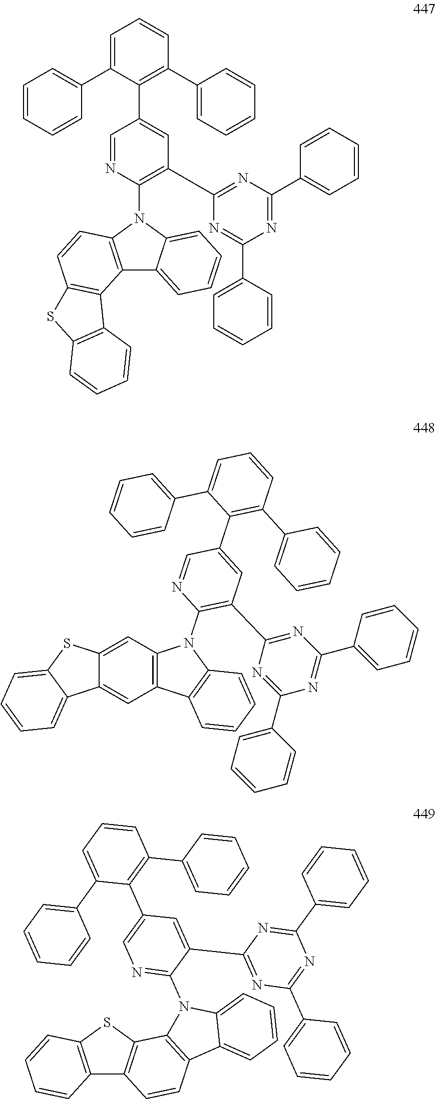 [SMILES: c1ccc(-c2nc(-c3ccccc3)nc(-c3cc(-c4c(-c5ccccc5)cccc4-c4ccccc4)cnc3-n3c4ccccc4c4c5c(ccc43)sc3ccccc35)n2)cc1 ; c1ccc(-c2nc(-c3ccccc3)nc(-c3cc(-c4c(-c5ccccc5)cccc4-c4ccccc4)cnc3-n3c4ccccc4c4cc5c(cc43)sc3ccccc35)n2)cc1 ; c1ccc(-c2nc(-c3ccccc3)nc(-c3cc(-c4c(-c5ccccc5)cccc4-c4ccccc4)cnc3-n3c4ccccc4c4ccc5c6ccccc6sc5c43)n2)cc1]

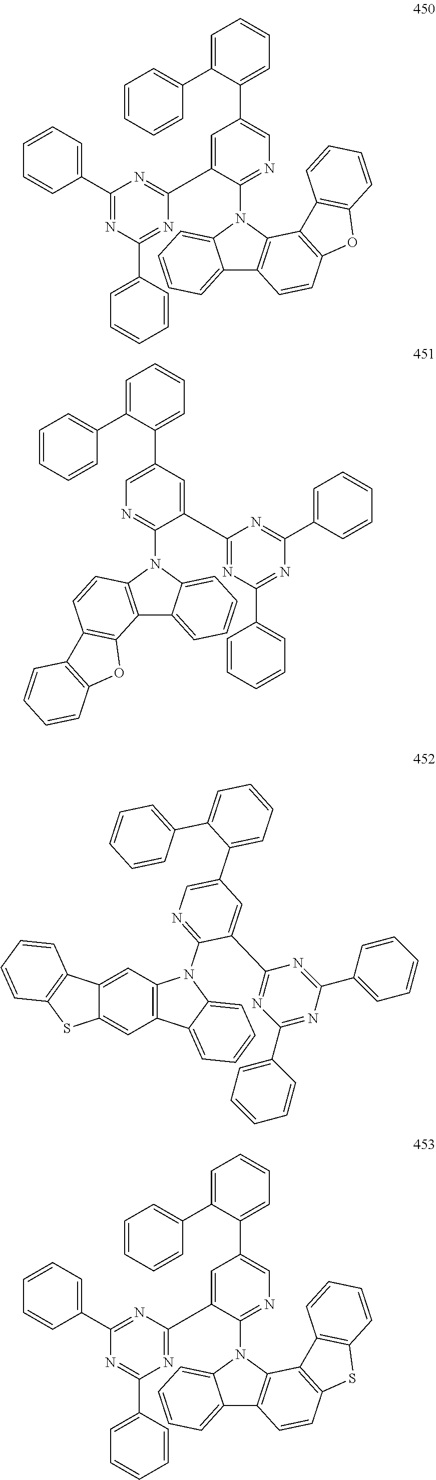

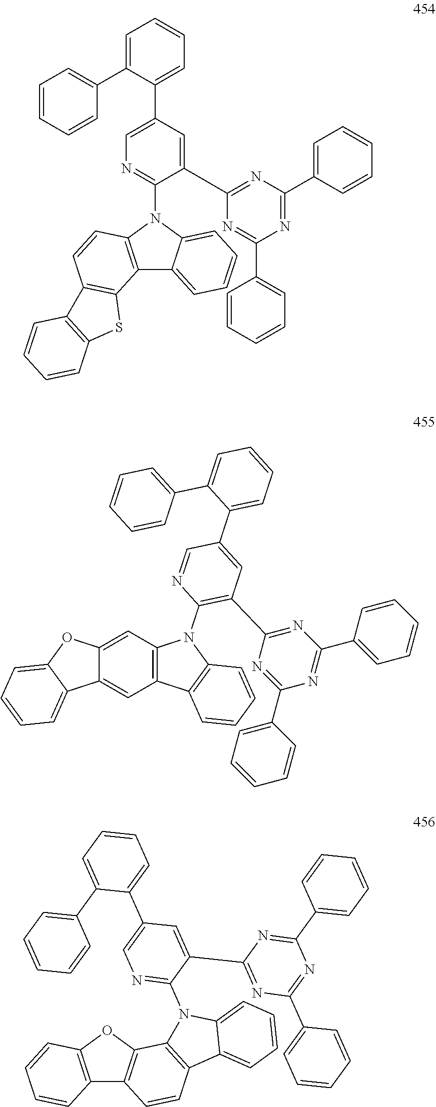 [SMILES: c1ccc(-c2nc(-c3ccccc3)nc(-c3cc(-c4ccccc4-c4ccccc4)cnc3-n3c4ccccc4c4c5sc6ccccc6c5ccc43)n2)cc1 ; c1ccc(-c2nc(-c3ccccc3)nc(-c3cc(-c4ccccc4-c4ccccc4)cnc3-n3c4ccccc4c4cc5c(cc43)oc3ccccc35)n2)cc1 ; c1ccc(-c2nc(-c3ccccc3)nc(-c3cc(-c4ccccc4-c4ccccc4)cnc3-n3c4ccccc4c4ccc5c6ccccc6oc5c43)n2)cc1]

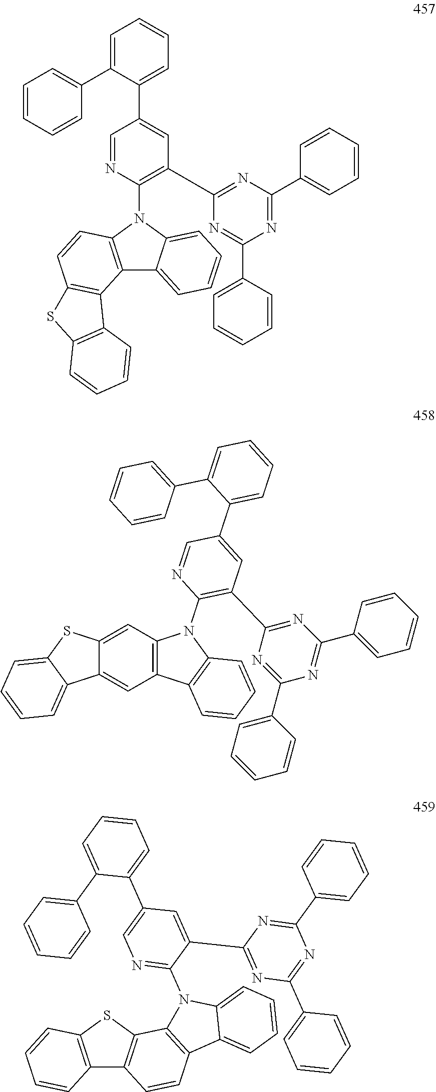 [SMILES: c1ccc(-c2nc(-c3ccccc3)nc(-c3cc(-c4ccccc4-c4ccccc4)cnc3-n3c4ccccc4c4c5c(ccc43)sc3ccccc35)n2)cc1 ; c1ccc(-c2nc(-c3ccccc3)nc(-c3cc(-c4ccccc4-c4ccccc4)cnc3-n3c4ccccc4c4cc5c(cc43)sc3ccccc35)n2)cc1 ; c1ccc(-c2nc(-c3ccccc3)nc(-c3cc(-c4ccccc4-c4ccccc4)cnc3-n3c4ccccc4c4ccc5c6ccccc6sc5c43)n2)cc1]

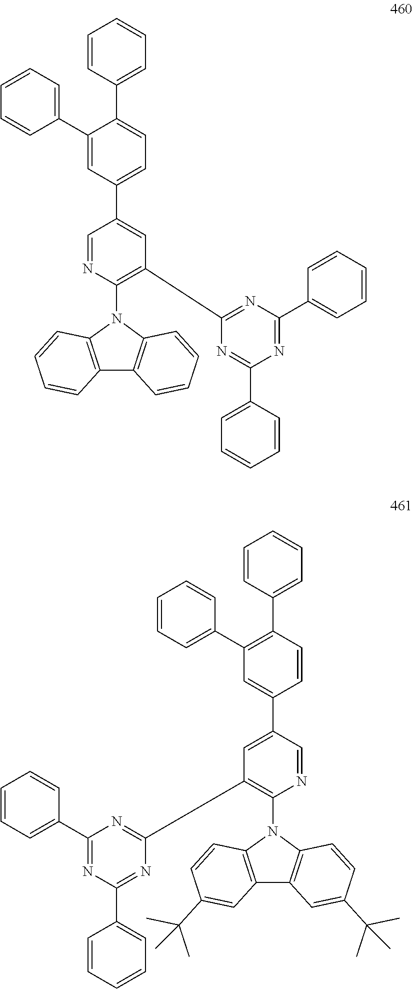 [SMILES: c1ccc(-c2nc(-c3ccccc3)nc(-c3cc(-c4ccc(-c5ccccc5)c(-c5ccccc5)c4)cnc3-n3c4ccccc4c4ccccc43)n2)cc1 ; CC(C)(C)c1ccc2c(c1)c1cc(C(C)(C)C)ccc1n2-c1ncc(-c2ccc(-c3ccccc3)c(-c3ccccc3)c2)cc1-c1nc(-c2ccccc2)nc(-c2ccccc2)n1]

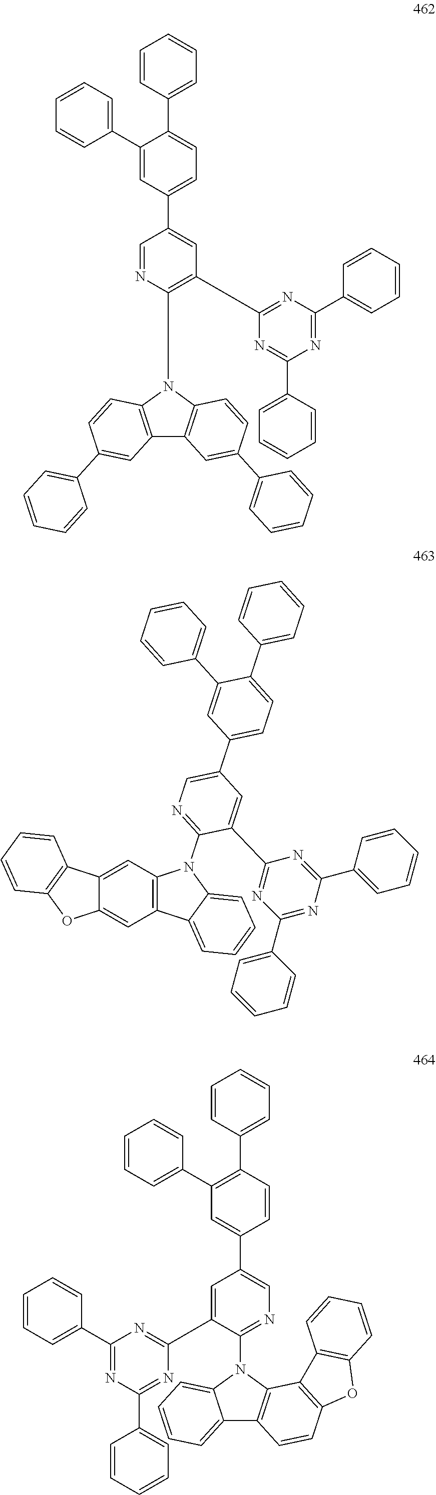 [SMILES: c1ccc(-c2ccc3c(c2)c2cc(-c4ccccc4)ccc2n3-c2ncc(-c3ccc(-c4ccccc4)c(-c4ccccc4)c3)cc2-c2nc(-c3ccccc3)nc(-c3ccccc3)n2)cc1 ; c1ccc(-c2nc(-c3ccccc3)nc(-c3cc(-c4ccc(-c5ccccc5)c(-c5ccccc5)c4)cnc3-n3c4ccccc4c4cc5oc6ccccc6c5cc43)n2)cc1 ; c1ccc(-c2nc(-c3ccccc3)nc(-c3cc(-c4ccc(-c5ccccc5)c(-c5ccccc5)c4)cnc3-n3c4ccccc4c4ccc5oc6ccccc6c5c43)n2)cc1]

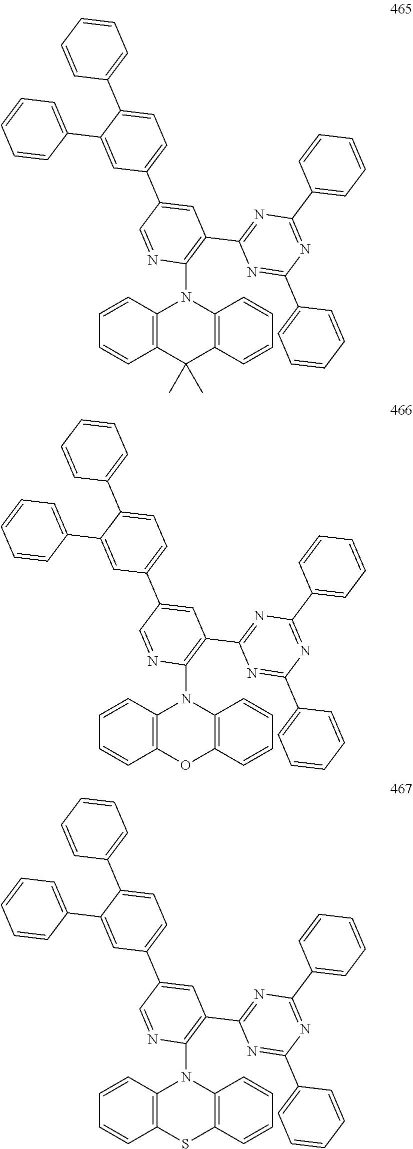

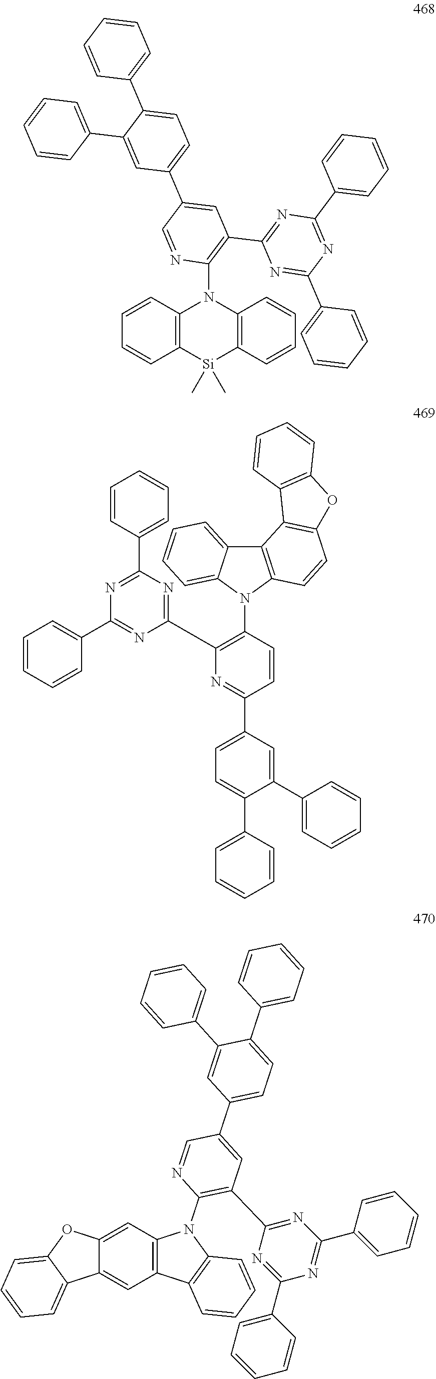 [SMILES: C[Si]1(C)c2ccccc2N(c2ncc(-c3ccc(-c4ccccc4)c(-c4ccccc4)c3)cc2-c2nc(-c3ccccc3)nc(-c3ccccc3)n2)c2ccccc21 ; c1ccc(-c2nc(-c3ccccc3)nc(-c3nc(-c4ccc(-c5ccccc5)c(-c5ccccc5)c4)ccc3-n3c4ccccc4c4c5c(ccc43)oc3ccccc35)n2)cc1 ; c1ccc(-c2nc(-c3ccccc3)nc(-c3cc(-c4ccc(-c5ccccc5)c(-c5ccccc5)c4)cnc3-n3c4ccccc4c4cc5c(cc43)oc3ccccc35)n2)cc1]

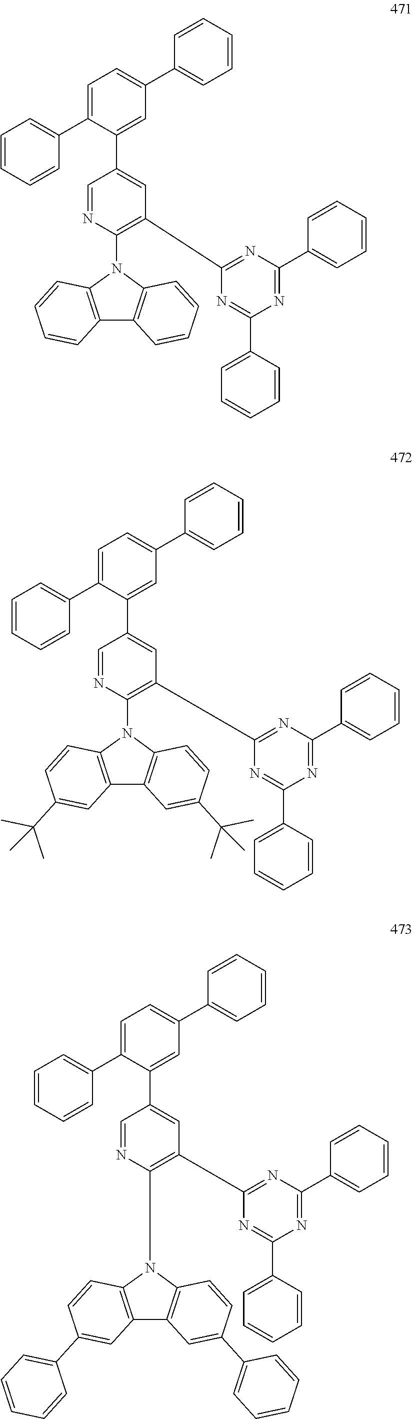 [SMILES: c1ccc(-c2ccc(-c3ccccc3)c(-c3cnc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ncc(-c2cc(-c3ccccc3)ccc2-c2ccccc2)cc1-c1nc(-c2ccccc2)nc(-c2ccccc2)n1 ; c1ccc(-c2ccc(-c3ccccc3)c(-c3cnc(-n4c5ccc(-c6ccccc6)cc5c5cc(-c6ccccc6)ccc54)c(-c4nc(-c5ccccc5)nc(-c5ccccc5)n4)c3)c2)cc1]

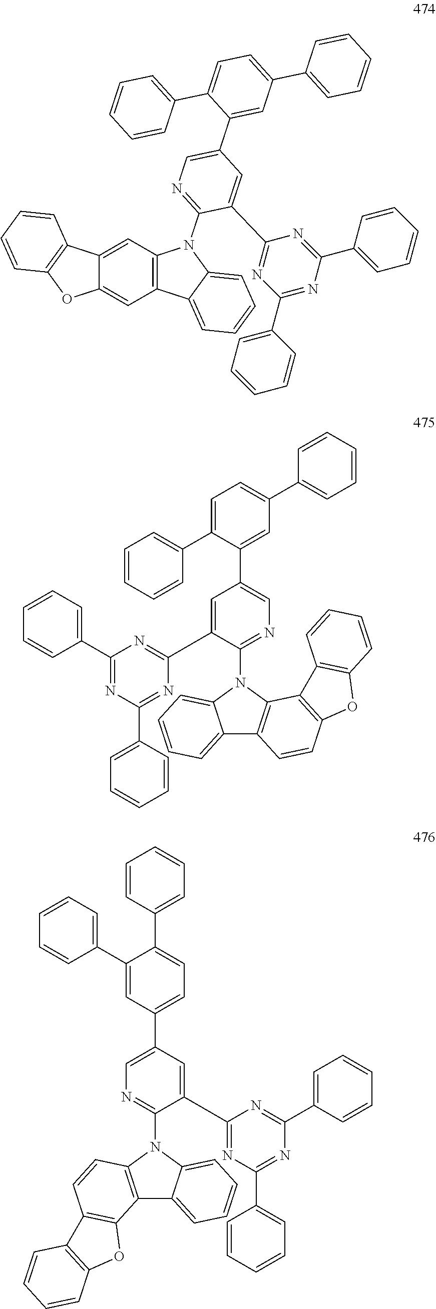

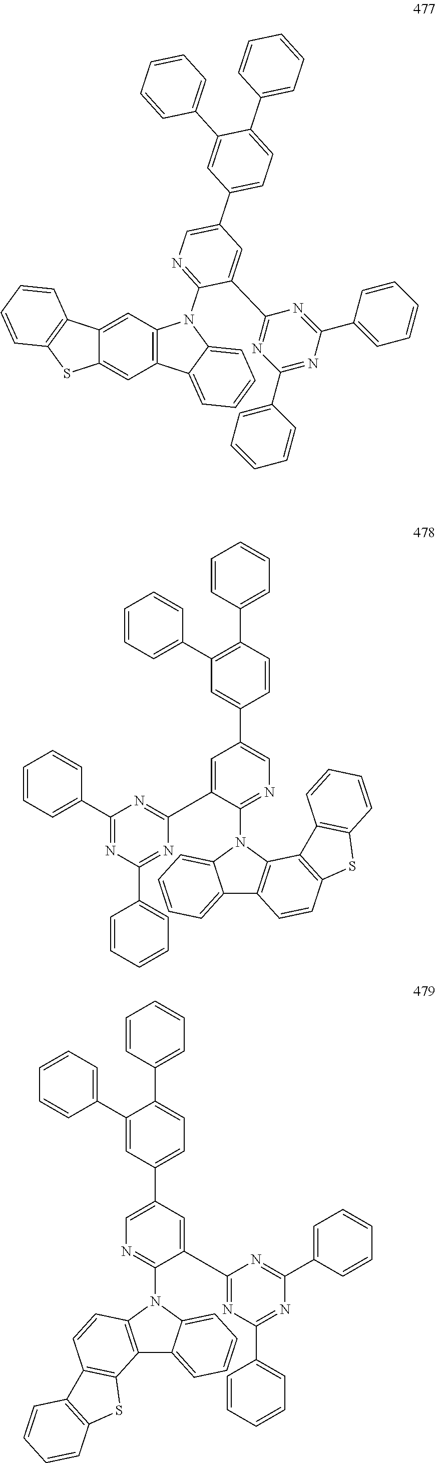

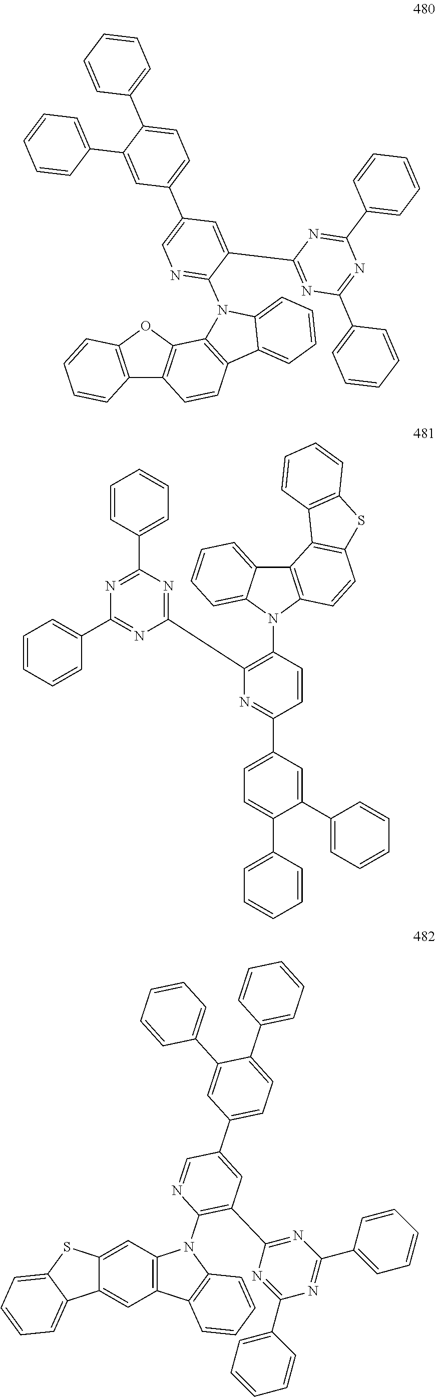

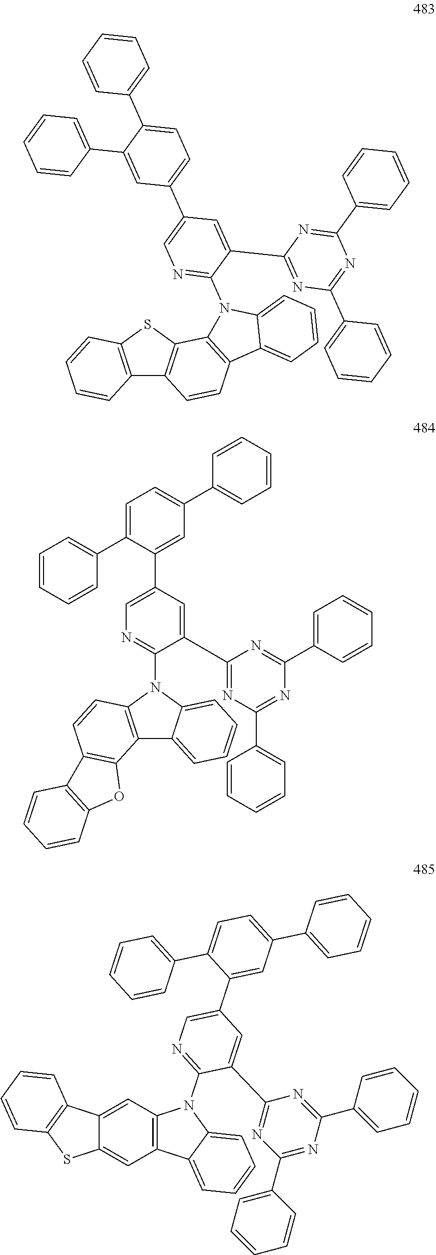 [SMILES: c1ccc(-c2nc(-c3ccccc3)nc(-c3cc(-c4ccc(-c5ccccc5)c(-c5ccccc5)c4)cnc3-n3c4ccccc4c4ccc5c6ccccc6sc5c43)n2)cc1 ; c1ccc(-c2ccc(-c3ccccc3)c(-c3cnc(-n4c5ccccc5c5c6oc7ccccc7c6ccc54)c(-c4nc(-c5ccccc5)nc(-c5ccccc5)n4)c3)c2)cc1 ; c1ccc(-c2ccc(-c3ccccc3)c(-c3cnc(-n4c5ccccc5c5cc6sc7ccccc7c6cc54)c(-c4nc(-c5ccccc5)nc(-c5ccccc5)n4)c3)c2)cc1]

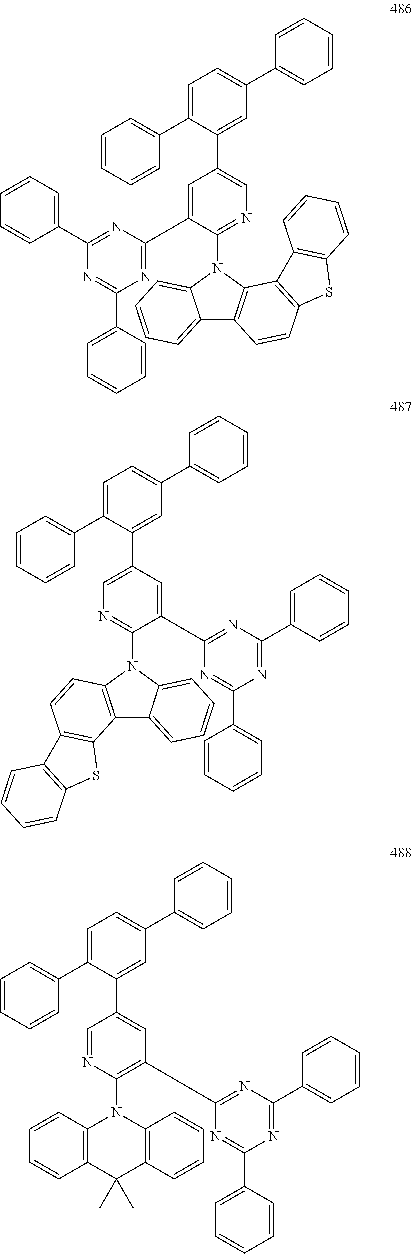 [SMILES: c1ccc(-c2ccc(-c3ccccc3)c(-c3cnc(-n4c5ccccc5c5ccc6sc7ccccc7c6c54)c(-c4nc(-c5ccccc5)nc(-c5ccccc5)n4)c3)c2)cc1 ; c1ccc(-c2ccc(-c3ccccc3)c(-c3cnc(-n4c5ccccc5c5c6sc7ccccc7c6ccc54)c(-c4nc(-c5ccccc5)nc(-c5ccccc5)n4)c3)c2)cc1 ; CC1(C)c2ccccc2N(c2ncc(-c3cc(-c4ccccc4)ccc3-c3ccccc3)cc2-c2nc(-c3ccccc3)nc(-c3ccccc3)n2)c2ccccc21]

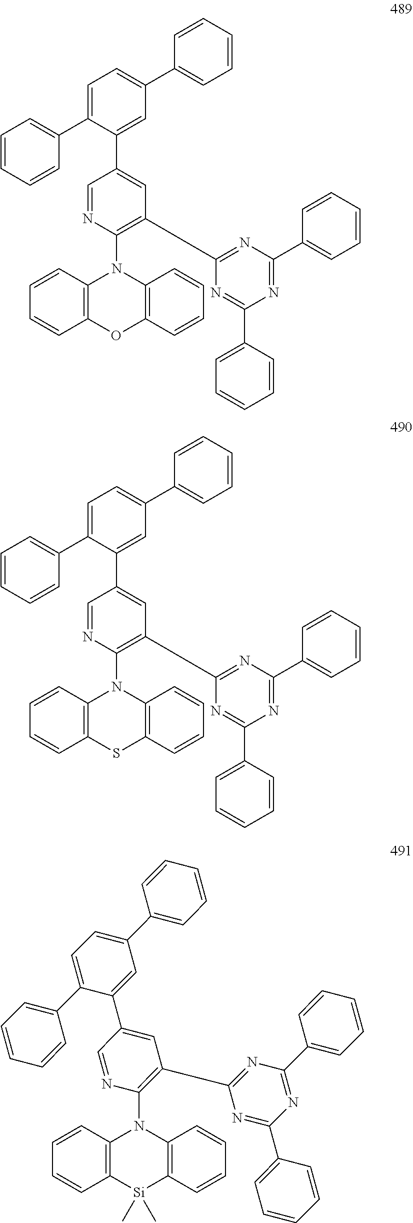

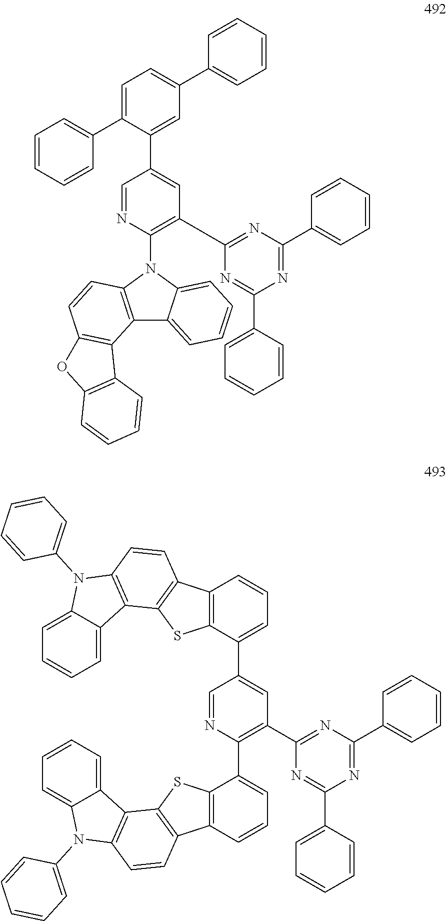 [SMILES: c1ccc(-c2ccc(-c3ccccc3)c(-c3cnc(-n4c5ccccc5c5c6c(ccc54)oc4ccccc46)c(-c4nc(-c5ccccc5)nc(-c5ccccc5)n4)c3)c2)cc1 ; c1ccc(-c2nc(-c3ccccc3)nc(-c3cc(-c4cccc5c4sc4c5ccc5c4c4ccccc4n5-c4ccccc4)cnc3-c3cccc4c3sc3c4ccc4c3c3ccccc3n4-c3ccccc3)n2)cc1]

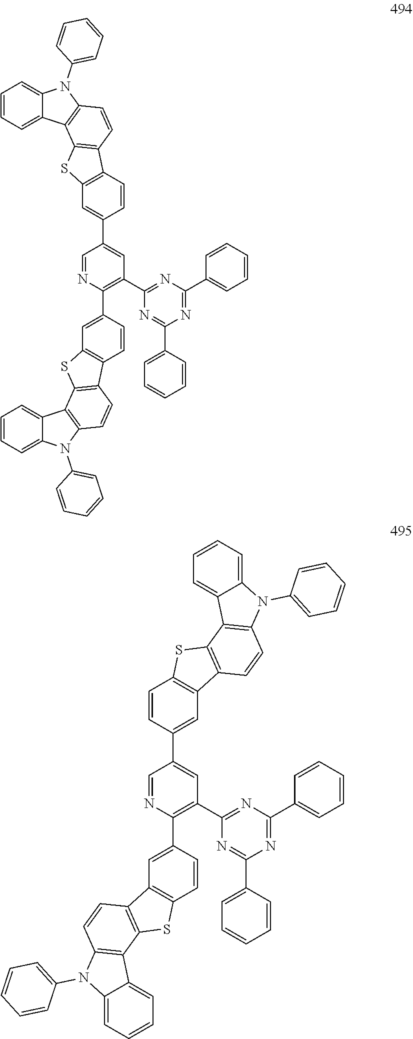 [SMILES: c1ccc(-c2nc(-c3ccccc3)nc(-c3cc(-c4ccc5c(c4)sc4c5ccc5c4c4ccccc4n5-c4ccccc4)cnc3-c3ccc4c(c3)sc3c4ccc4c3c3ccccc3n4-c3ccccc3)n2)cc1 ; c1ccc(-c2nc(-c3ccccc3)nc(-c3cc(-c4ccc5sc6c(ccc7c6c6ccccc6n7-c6ccccc6)c5c4)cnc3-c3ccc4sc5c(ccc6c5c5ccccc5n6-c5ccccc5)c4c3)n2)cc1]

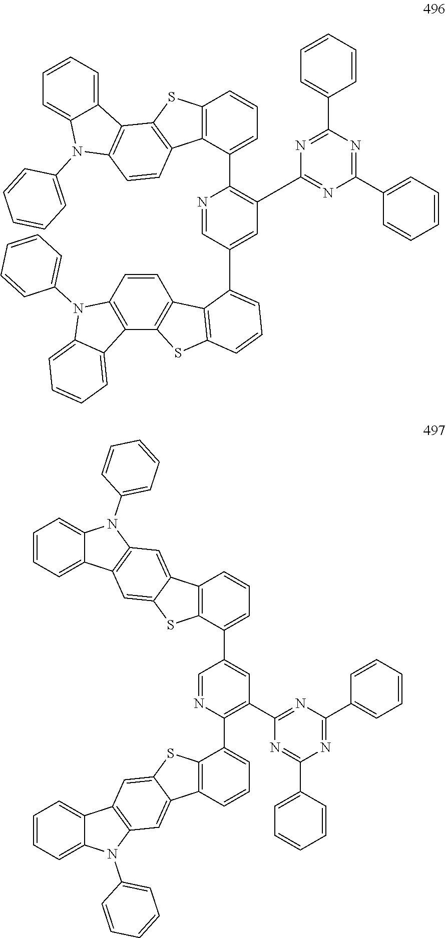 [SMILES: c1ccc(-c2nc(-c3ccccc3)nc(-c3cc(-c4cccc5sc6c(ccc7c6c6ccccc6n7-c6ccccc6)c45)cnc3-c3cccc4sc5c(ccc6c5c5ccccc5n6-c5ccccc5)c34)n2)cc1 ; c1ccc(-c2nc(-c3ccccc3)nc(-c3cc(-c4cccc5c4sc4cc6c7ccccc7n(-c7ccccc7)c6cc45)cnc3-c3cccc4c3sc3cc5c6ccccc6n(-c6ccccc6)c5cc34)n2)cc1]

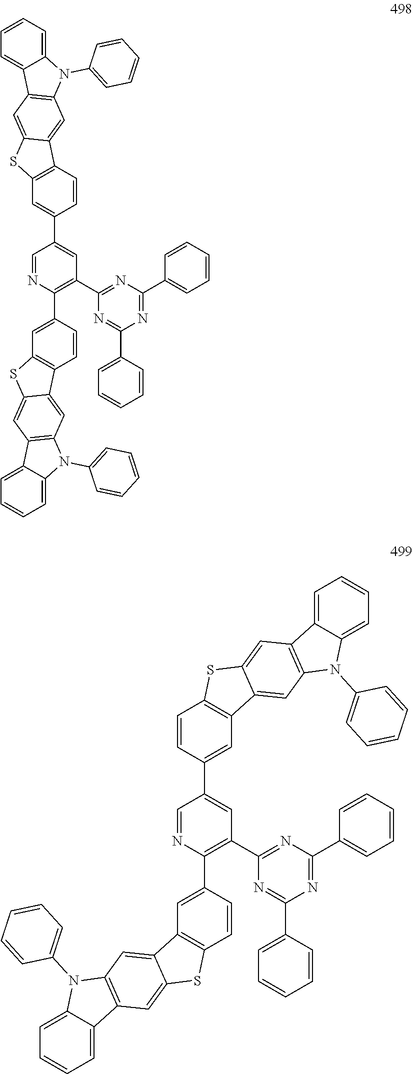 [SMILES: c1ccc(-c2nc(-c3ccccc3)nc(-c3cc(-c4ccc5c(c4)sc4cc6c7ccccc7n(-c7ccccc7)c6cc45)cnc3-c3ccc4c(c3)sc3cc5c6ccccc6n(-c6ccccc6)c5cc34)n2)cc1 ; c1ccc(-c2nc(-c3ccccc3)nc(-c3cc(-c4ccc5sc6cc7c8ccccc8n(-c8ccccc8)c7cc6c5c4)cnc3-c3ccc4sc5cc6c7ccccc7n(-c7ccccc7)c6cc5c4c3)n2)cc1]

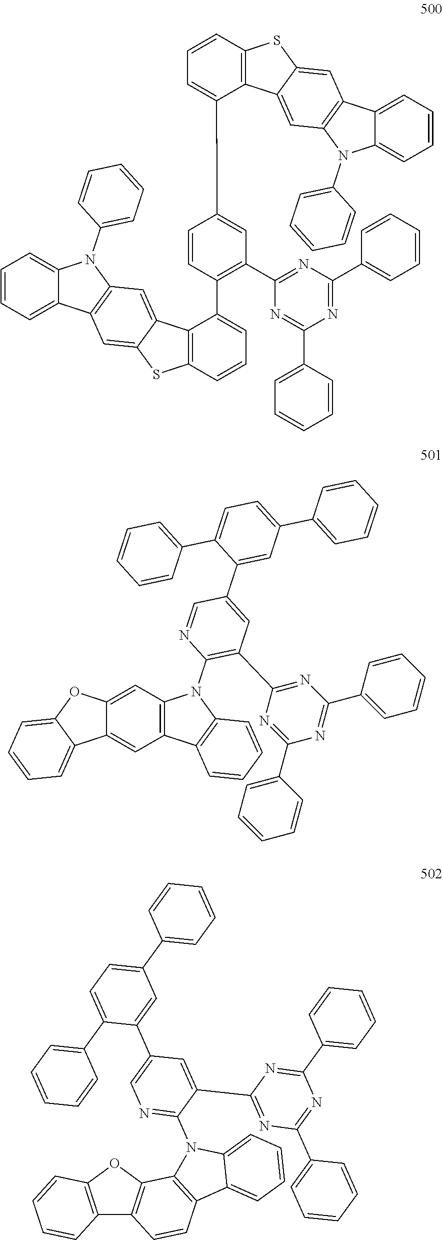 [SMILES: c1ccc(-c2nc(-c3ccccc3)nc(-c3cc(-c4cccc5sc6cc7c8ccccc8n(-c8ccccc8)c7cc6c45)ccc3-c3cccc4sc5cc6c7ccccc7n(-c7ccccc7)c6cc5c34)n2)cc1 ; c1ccc(-c2ccc(-c3ccccc3)c(-c3cnc(-n4c5ccccc5c5cc6c(cc54)oc4ccccc46)c(-c4nc(-c5ccccc5)nc(-c5ccccc5)n4)c3)c2)cc1 ; c1ccc(-c2ccc(-c3ccccc3)c(-c3cnc(-n4c5ccccc5c5ccc6c7ccccc7oc6c54)c(-c4nc(-c5ccccc5)nc(-c5ccccc5)n4)c3)c2)cc1]

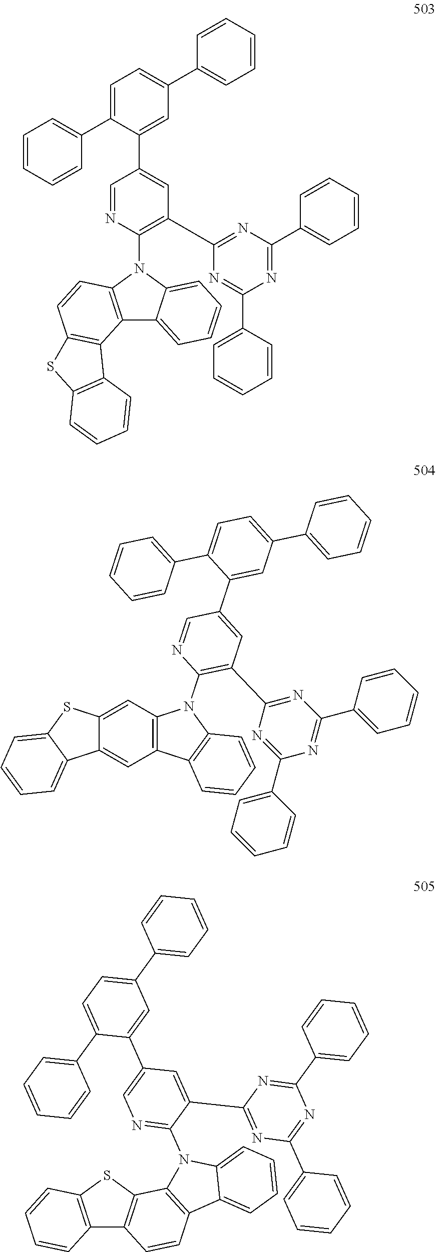 [SMILES: c1ccc(-c2ccc(-c3ccccc3)c(-c3cnc(-n4c5ccccc5c5c6c(ccc54)sc4ccccc46)c(-c4nc(-c5ccccc5)nc(-c5ccccc5)n4)c3)c2)cc1 ; c1ccc(-c2ccc(-c3ccccc3)c(-c3cnc(-n4c5ccccc5c5cc6c(cc54)sc4ccccc46)c(-c4nc(-c5ccccc5)nc(-c5ccccc5)n4)c3)c2)cc1 ; c1ccc(-c2ccc(-c3ccccc3)c(-c3cnc(-n4c5ccccc5c5ccc6c7ccccc7sc6c54)c(-c4nc(-c5ccccc5)nc(-c5ccccc5)n4)c3)c2)cc1]

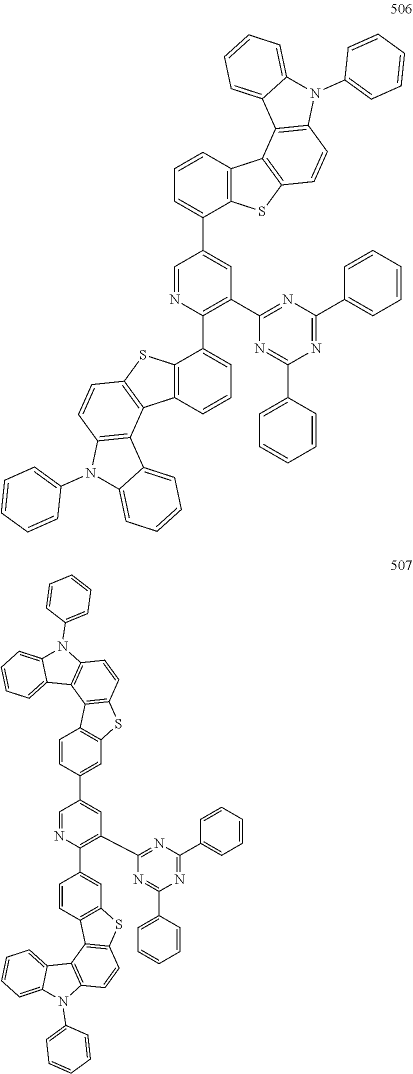 [SMILES: c1ccc(-c2nc(-c3ccccc3)nc(-c3cc(-c4cccc5c4sc4ccc6c(c7ccccc7n6-c6ccccc6)c45)cnc3-c3cccc4c3sc3ccc5c(c6ccccc6n5-c5ccccc5)c34)n2)cc1 ; c1ccc(-c2nc(-c3ccccc3)nc(-c3cc(-c4ccc5c(c4)sc4ccc6c(c7ccccc7n6-c6ccccc6)c45)cnc3-c3ccc4c(c3)sc3ccc5c(c6ccccc6n5-c5ccccc5)c34)n2)cc1]

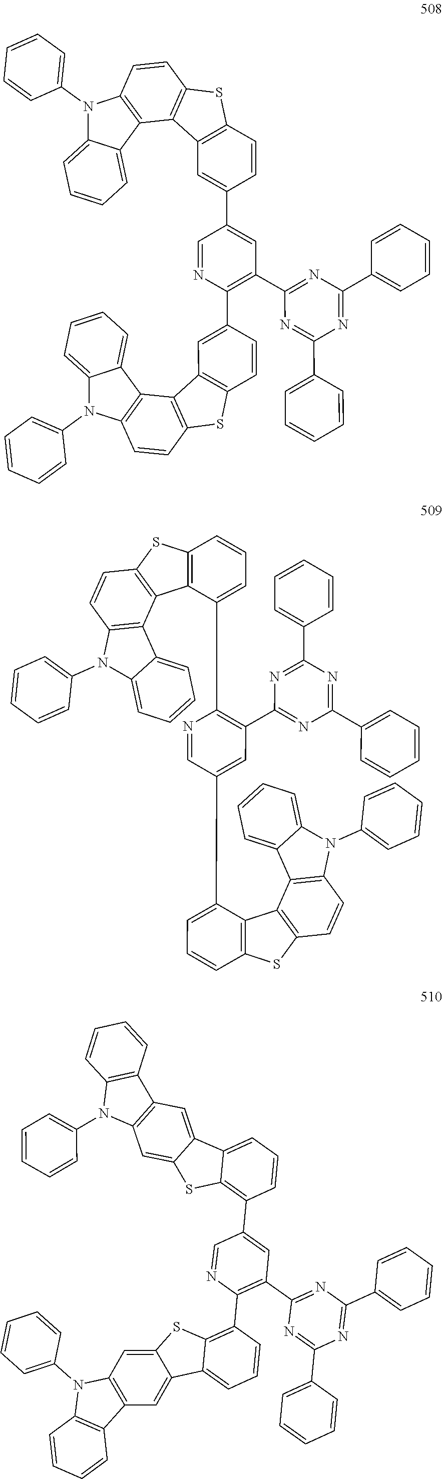 [SMILES: c1ccc(-c2nc(-c3ccccc3)nc(-c3cc(-c4ccc5sc6ccc7c(c8ccccc8n7-c7ccccc7)c6c5c4)cnc3-c3ccc4sc5ccc6c(c7ccccc7n6-c6ccccc6)c5c4c3)n2)cc1 ; c1ccc(-c2nc(-c3ccccc3)nc(-c3cc(-c4cccc5sc6ccc7c(c8ccccc8n7-c7ccccc7)c6c45)cnc3-c3cccc4sc5ccc6c(c7ccccc7n6-c6ccccc6)c5c34)n2)cc1 ; c1ccc(-c2nc(-c3ccccc3)nc(-c3cc(-c4cccc5c4sc4cc6c(cc45)c4ccccc4n6-c4ccccc4)cnc3-c3cccc4c3sc3cc5c(cc34)c3ccccc3n5-c3ccccc3)n2)cc1]

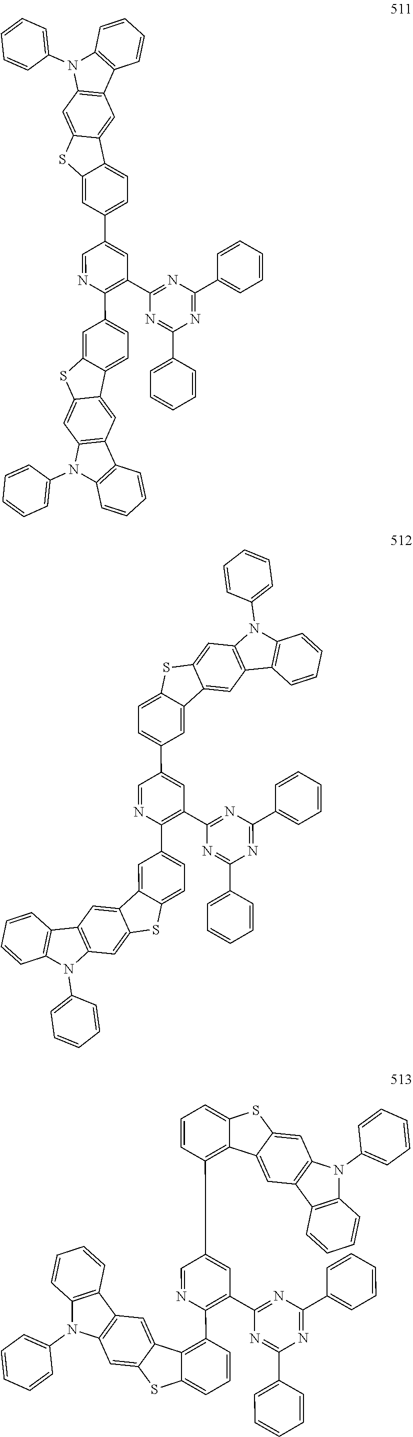 [SMILES: c1ccc(-c2nc(-c3ccccc3)nc(-c3cc(-c4ccc5c(c4)sc4cc6c(cc45)c4ccccc4n6-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccc5sc6cc7c(cc6c5c4)c4ccccc4n7-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4cccc5sc6cc7c(cc6c45)c4ccccc4n7-c4ccccc4)cnc3-c3cccc4sc5cc6c(cc5c34)c3ccccc3n6-c3ccccc3)n2)cc1]

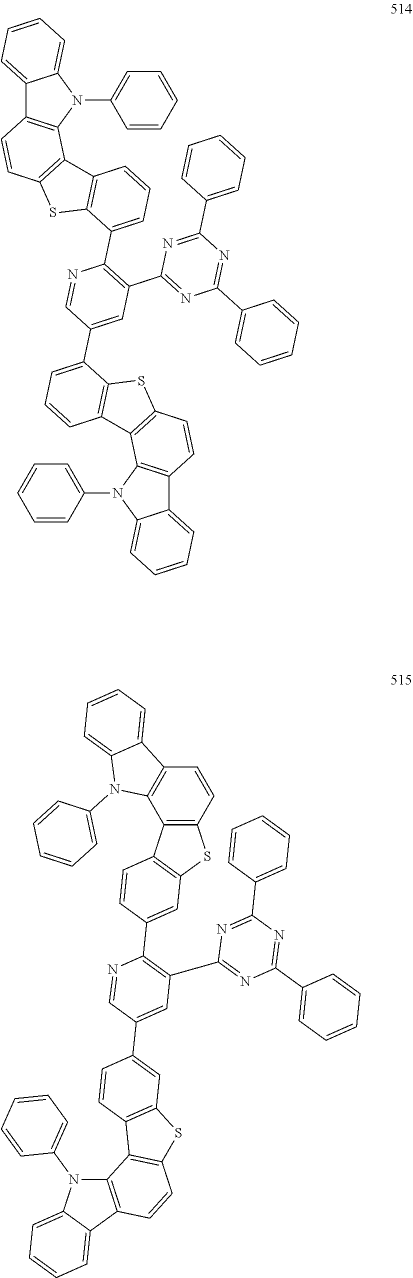 [SMILES: c1ccc(-c2nc(-c3ccccc3)nc(-c3cc(-c4cccc5c4sc4ccc6c7ccccc7n(-c7ccccc7)c6c45)cnc3-c3cccc4c3sc3ccc5c6ccccc6n(-c6ccccc6)c5c34)n2)cc1 ; c1ccc(-c2nc(-c3ccccc3)nc(-c3cc(-c4ccc5c(c4)sc4ccc6c7ccccc7n(-c7ccccc7)c6c45)cnc3-c3ccc4c(c3)sc3ccc5c6ccccc6n(-c6ccccc6)c5c34)n2)cc1]

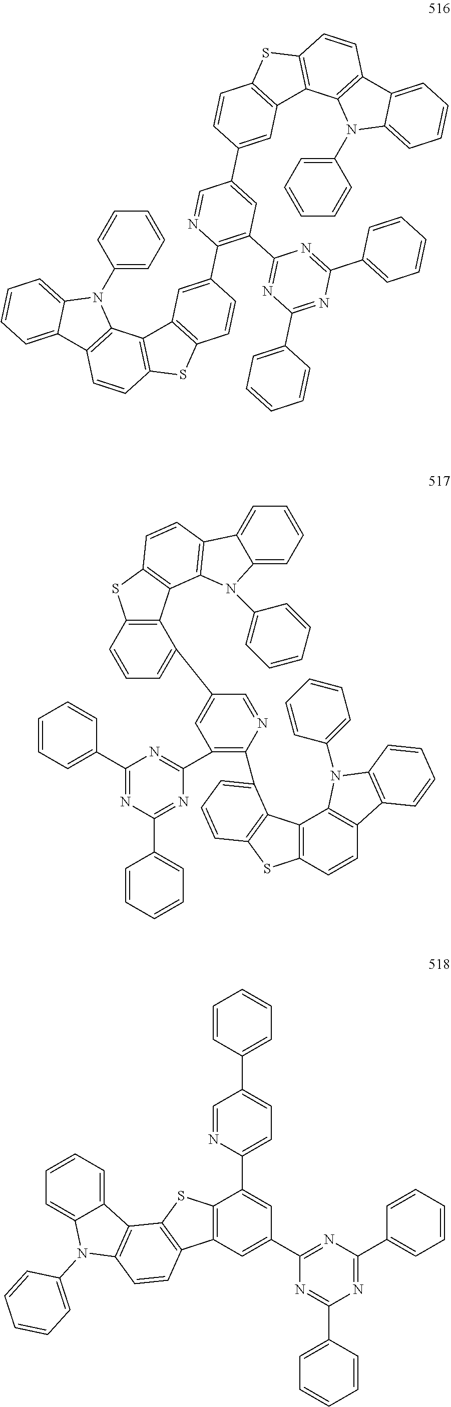 [SMILES: c1ccc(-c2nc(-c3ccccc3)nc(-c3cc(-c4ccc5sc6ccc7c8ccccc8n(-c8ccccc8)c7c6c5c4)cnc3-c3ccc4sc5ccc6c7ccccc7n(-c7ccccc7)c6c5c4c3)n2)cc1 ; c1ccc(-c2nc(-c3ccccc3)nc(-c3cc(-c4cccc5sc6ccc7c8ccccc8n(-c8ccccc8)c7c6c45)cnc3-c3cccc4sc5ccc6c7ccccc7n(-c7ccccc7)c6c5c34)n2)cc1 ; c1ccc(-c2ccc(-c3cc(-c4nc(-c5ccccc5)nc(-c5ccccc5)n4)cc4c3sc3c4ccc4c3c3ccccc3n4-c3ccccc3)nc2)cc1]

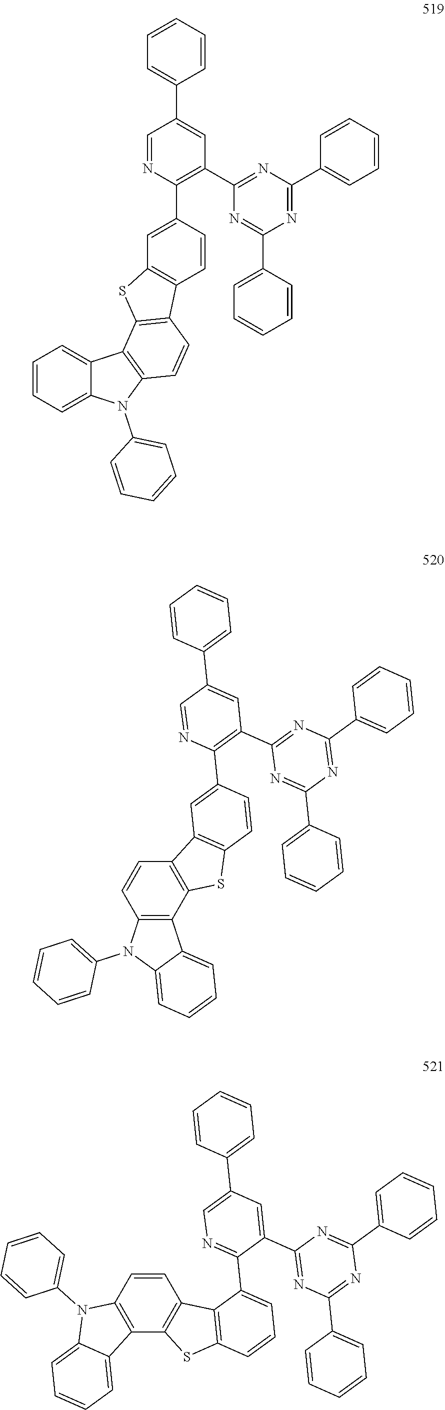 [SMILES: c1ccc(-c2cnc(-c3ccc4c(c3)sc3c4ccc4c3c3ccccc3n4-c3ccccc3)c(-c3nc(-c4ccccc4)nc(-c4ccccc4)n3)c2)cc1 ; c1ccc(-c2cnc(-c3ccc4sc5c(ccc6c5c5ccccc5n6-c5ccccc5)c4c3)c(-c3nc(-c4ccccc4)nc(-c4ccccc4)n3)c2)cc1 ; c1ccc(-c2cnc(-c3cccc4sc5c(ccc6c5c5ccccc5n6-c5ccccc5)c34)c(-c3nc(-c4ccccc4)nc(-c4ccccc4)n3)c2)cc1]

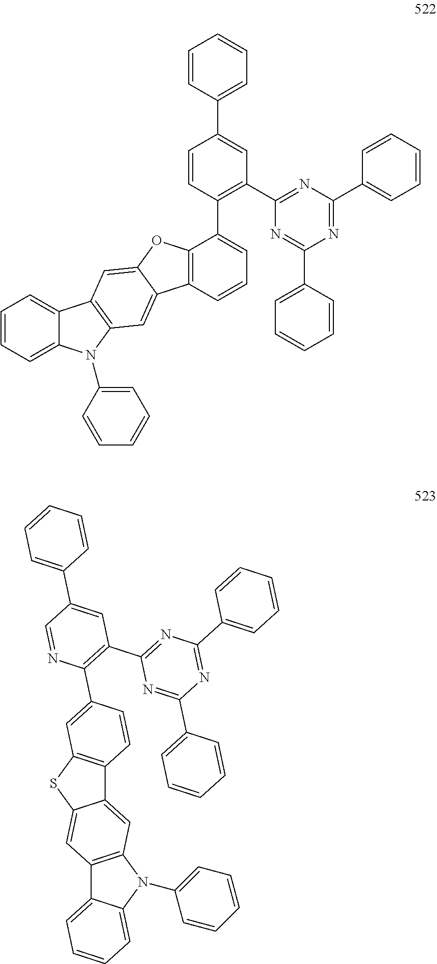 [SMILES: c1ccc(-c2ccc(-c3cccc4c3oc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4c(c3)sc3cc5c6ccccc6n(-c6ccccc6)c5cc34)c(-c3nc(-c4ccccc4)nc(-c4ccccc4)n3)c2)cc1]

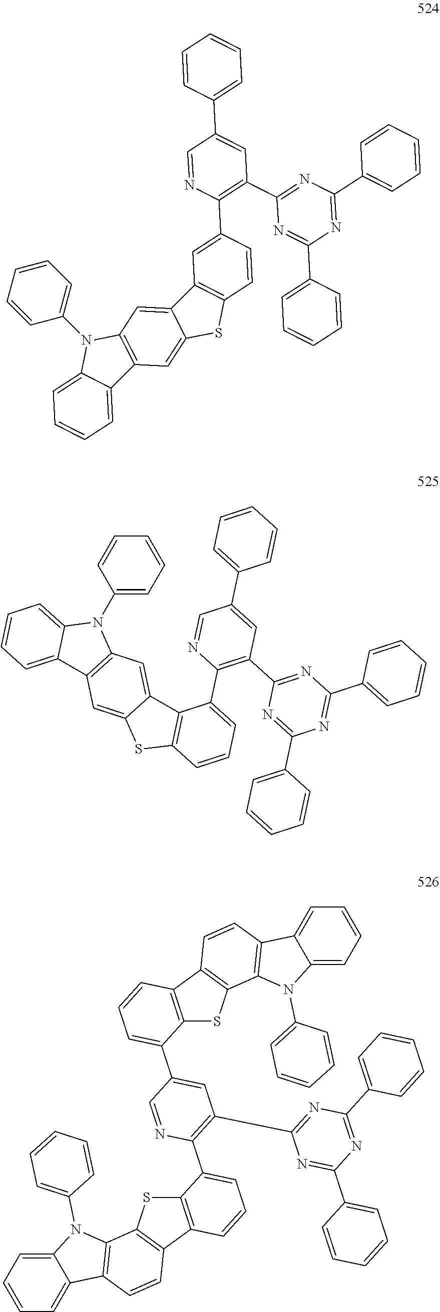 [SMILES: c1ccc(-c2cnc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2cnc(-c3cccc4sc5cc6c7ccccc7n(-c7ccccc7)c6cc5c34)c(-c3nc(-c4ccccc4)nc(-c4ccccc4)n3)c2)cc1 ; c1ccc(-c2nc(-c3ccccc3)nc(-c3cc(-c4cccc5c4sc4c5ccc5c6ccccc6n(-c6ccccc6)c54)cnc3-c3cccc4c3sc3c4ccc4c5ccccc5n(-c5ccccc5)c43)n2)cc1]

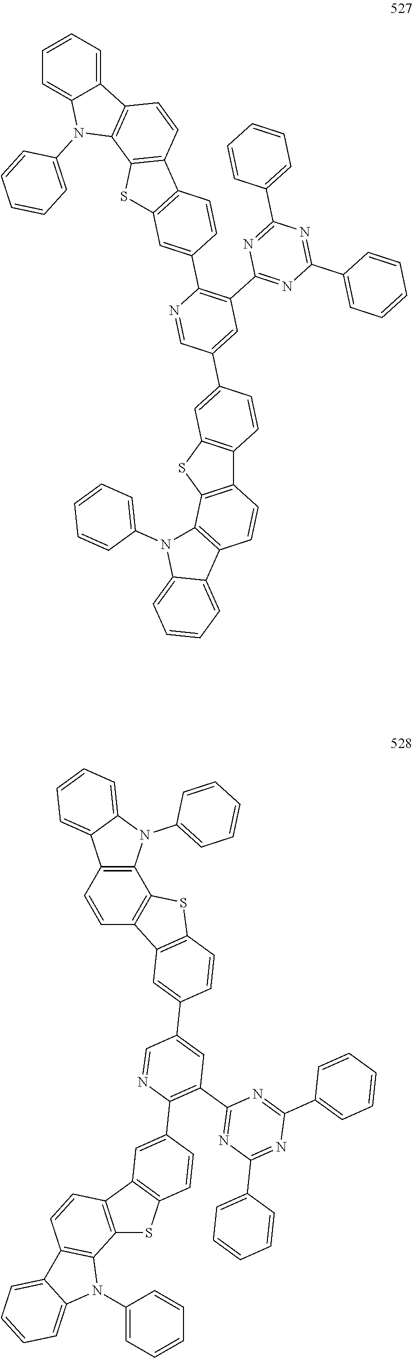 [SMILES: c1ccc(-c2nc(-c3ccccc3)nc(-c3cc(-c4ccc5c(c4)sc4c5ccc5c6ccccc6n(-c6ccccc6)c54)cnc3-c3ccc4c(c3)sc3c4ccc4c5ccccc5n(-c5ccccc5)c43)n2)cc1 ; c1ccc(-c2nc(-c3ccccc3)nc(-c3cc(-c4ccc5sc6c(ccc7c8ccccc8n(-c8ccccc8)c76)c5c4)cnc3-c3ccc4sc5c(ccc6c7ccccc7n(-c7ccccc7)c65)c4c3)n2)cc1]

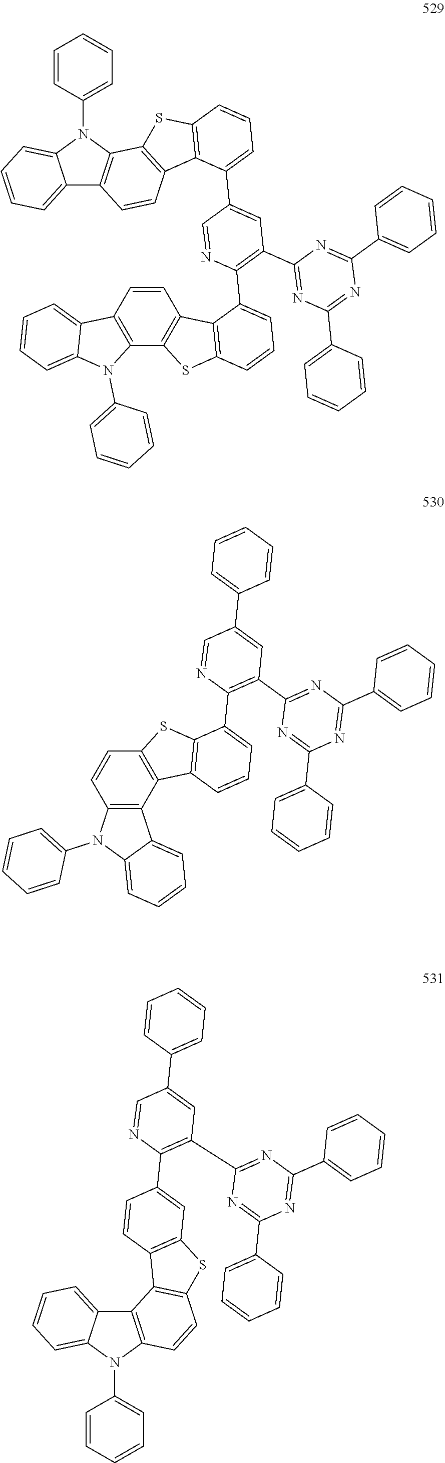 [SMILES: c1ccc(-c2nc(-c3ccccc3)nc(-c3cc(-c4cccc5sc6c(ccc7c8ccccc8n(-c8ccccc8)c76)c45)cnc3-c3cccc4sc5c(ccc6c7ccccc7n(-c7ccccc7)c65)c34)n2)cc1 ; c1ccc(-c2cnc(-c3cccc4c3sc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4c(c3)sc3ccc5c(c6ccccc6n5-c5ccccc5)c34)c(-c3nc(-c4ccccc4)nc(-c4ccccc4)n3)c2)cc1]

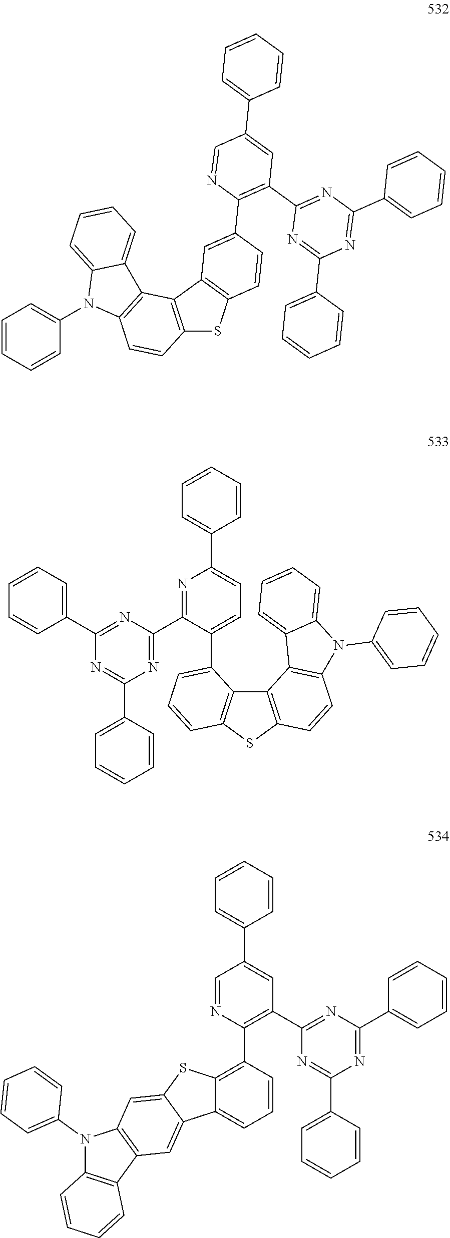 [SMILES: c1ccc(-c2cnc(-c3ccc4sc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4sc5ccc6c(c7ccccc7n6-c6ccccc6)c5c34)c(-c3nc(-c4ccccc4)nc(-c4ccccc4)n3)n2)cc1 ; c1ccc(-c2cnc(-c3cccc4c3sc3cc5c(cc34)c3ccccc3n5-c3ccccc3)c(-c3nc(-c4ccccc4)nc(-c4ccccc4)n3)c2)cc1]

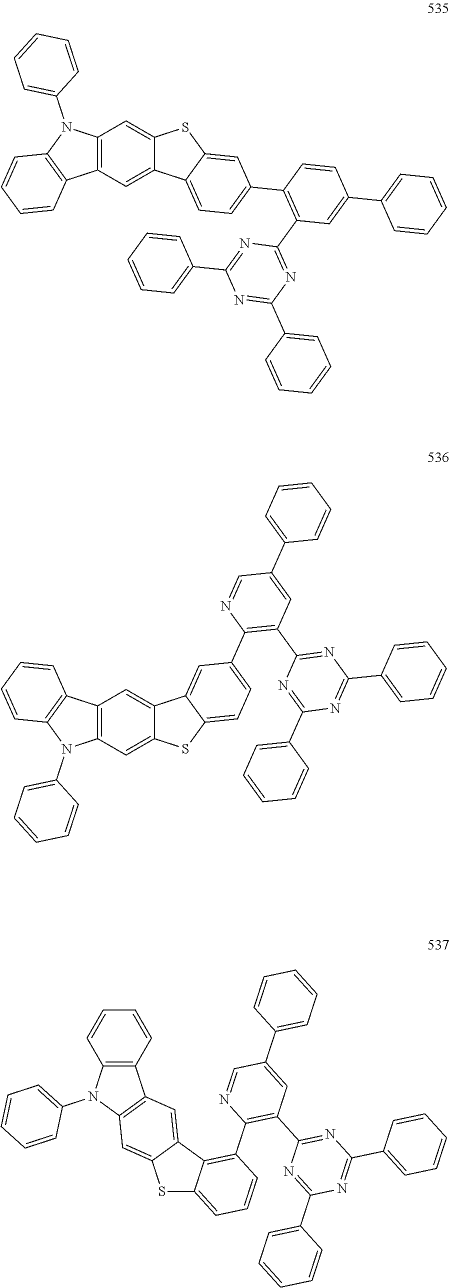 [SMILES: c1ccc(-c2ccc(-c3ccc4c(c3)sc3cc5c(cc34)c3ccccc3n5-c3ccccc3)c(-c3nc(-c4ccccc4)nc(-c4ccccc4)n3)c2)cc1 ; c1ccc(-c2cnc(-c3ccc4sc5cc6c(cc5c4c3)c3ccccc3n6-c3ccccc3)c(-c3nc(-c4ccccc4)nc(-c4ccccc4)n3)c2)cc1 ; c1ccc(-c2cnc(-c3cccc4sc5cc6c(cc5c34)c3ccccc3n6-c3ccccc3)c(-c3nc(-c4ccccc4)nc(-c4ccccc4)n3)c2)cc1]

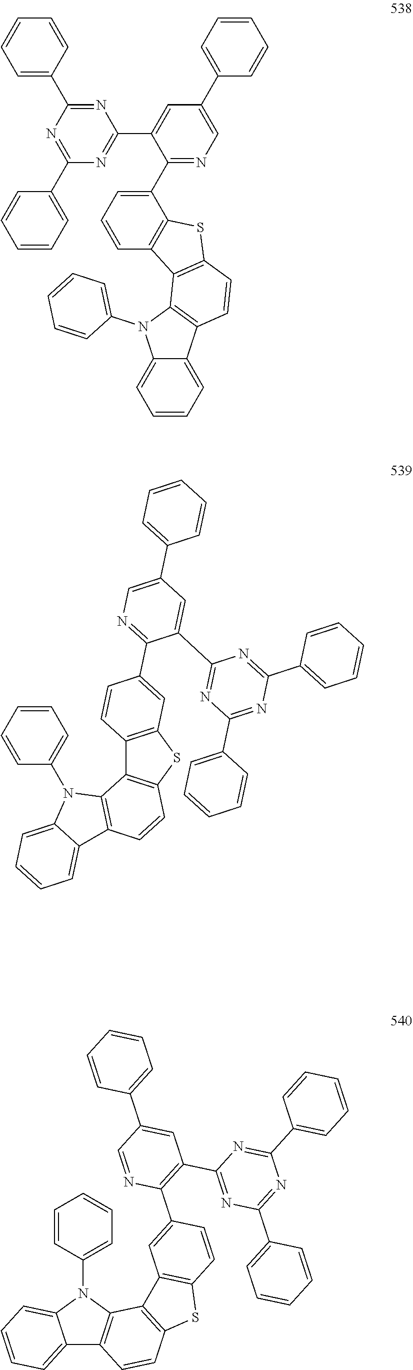 [SMILES: c1ccc(-c2cnc(-c3cccc4c3sc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4c(c3)sc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4sc5ccc6c7ccccc7n(-c7ccccc7)c6c5c4c3)c(-c3nc(-c4ccccc4)nc(-c4ccccc4)n3)c2)cc1]

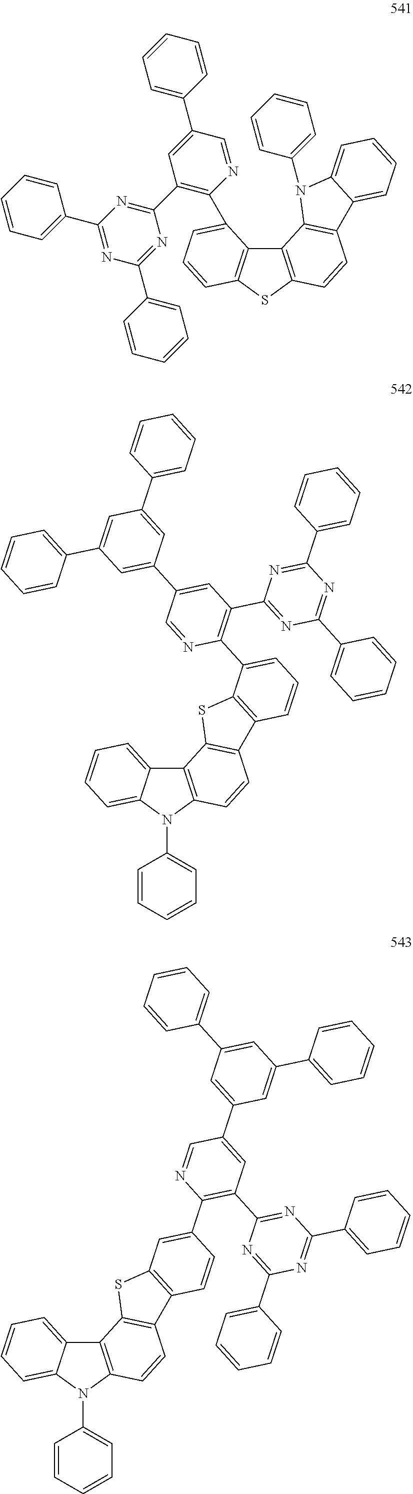 [SMILES: c1ccc(-c2cnc(-c3cccc4sc5ccc6c7ccccc7n(-c7ccccc7)c6c5c34)c(-c3nc(-c4ccccc4)nc(-c4ccccc4)n3)c2)cc1 ; c1ccc(-c2cc(-c3ccccc3)cc(-c3cnc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1]

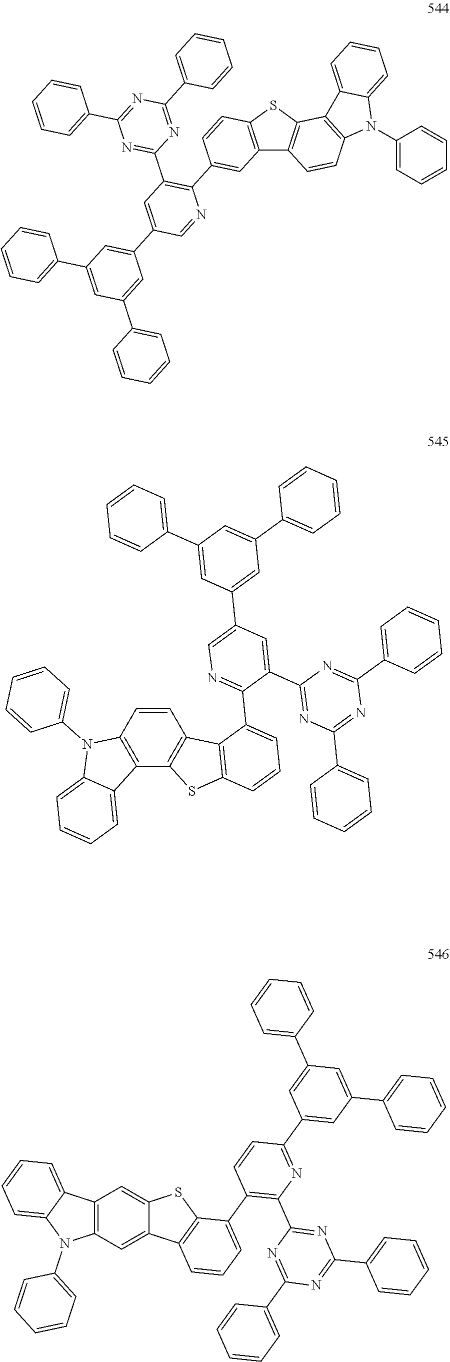 [SMILES: c1ccc(-c2cc(-c3ccccc3)cc(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3cnc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4cc6c7ccccc7n(-c7ccccc7)c6cc45)c(-c4nc(-c5ccccc5)nc(-c5ccccc5)n4)n3)c2)cc1]

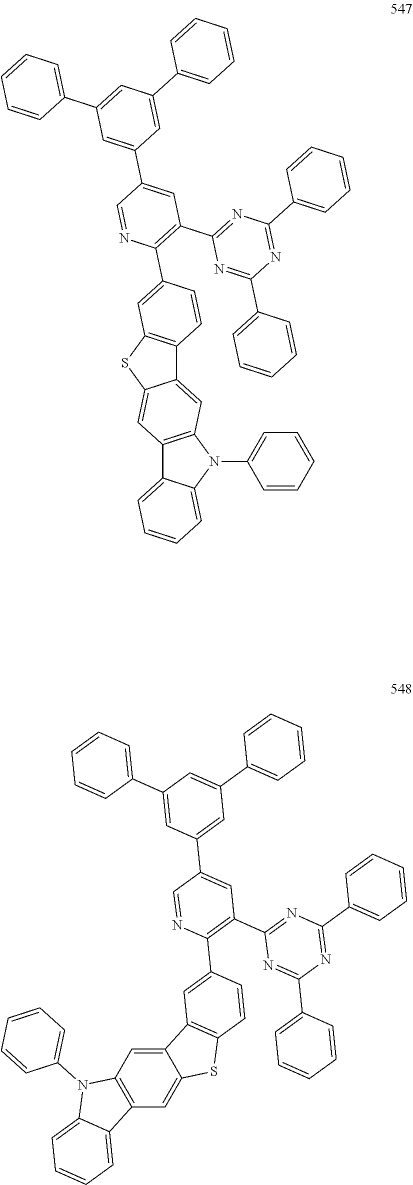 [SMILES: c1ccc(-c2cc(-c3ccccc3)cc(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1]

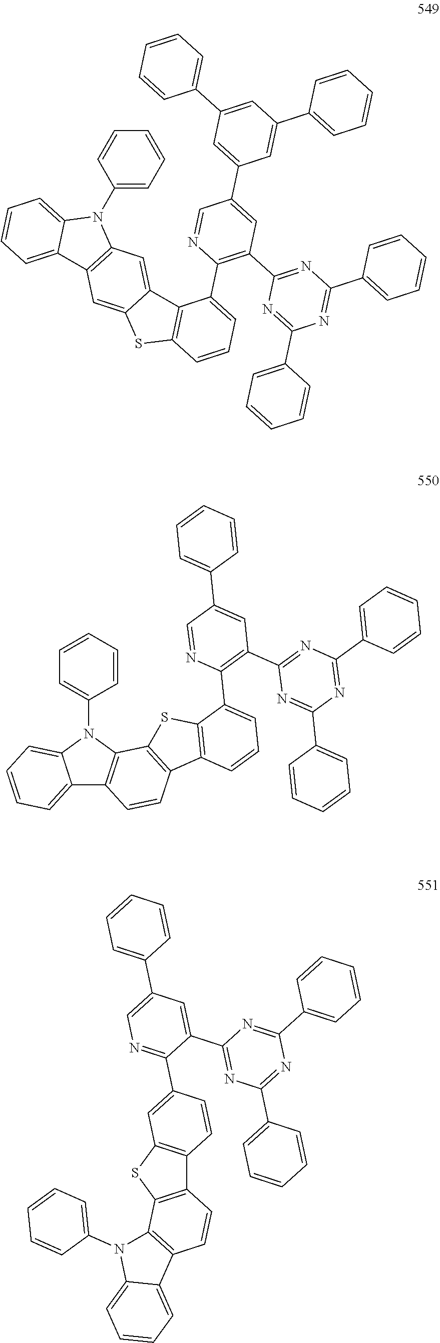 [SMILES: c1ccc(-c2cc(-c3ccccc3)cc(-c3cnc(-c4cccc5sc6cc7c8ccccc8n(-c8ccccc8)c7cc6c45)c(-c4nc(-c5ccccc5)nc(-c5ccccc5)n4)c3)c2)cc1 ; c1ccc(-c2cnc(-c3cccc4c3sc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4c(c3)sc3c4ccc4c5ccccc5n(-c5ccccc5)c43)c(-c3nc(-c4ccccc4)nc(-c4ccccc4)n3)c2)cc1]

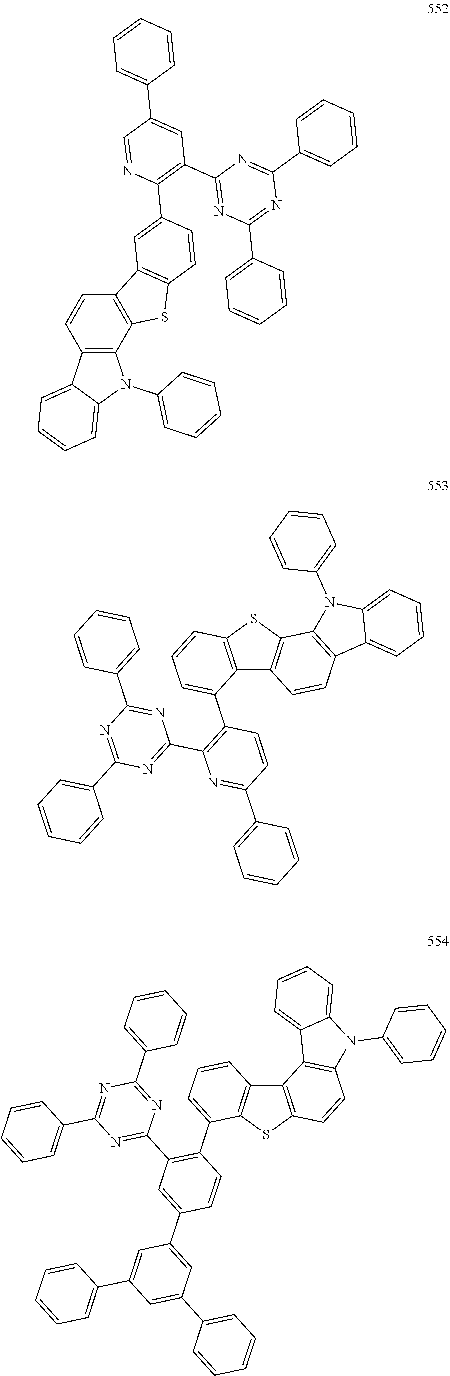 [SMILES: c1ccc(-c2cnc(-c3ccc4sc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4sc5c(ccc6c7ccccc7n(-c7ccccc7)c65)c34)c(-c3nc(-c4ccccc4)nc(-c4ccccc4)n3)n2)cc1 ; c1ccc(-c2cc(-c3ccccc3)cc(-c3ccc(-c4cccc5c4sc4ccc6c(c7ccccc7n6-c6ccccc6)c45)c(-c4nc(-c5ccccc5)nc(-c5ccccc5)n4)c3)c2)cc1]

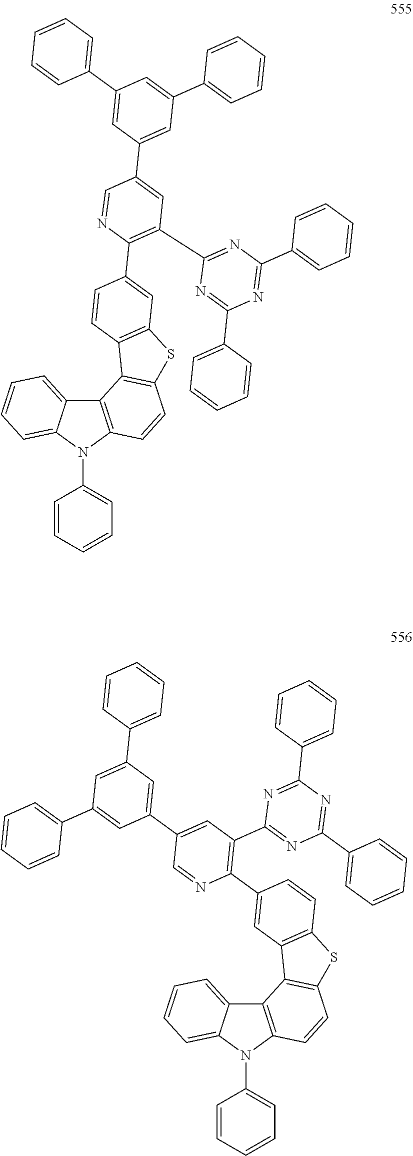 [SMILES: c1ccc(-c2cc(-c3ccccc3)cc(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1]

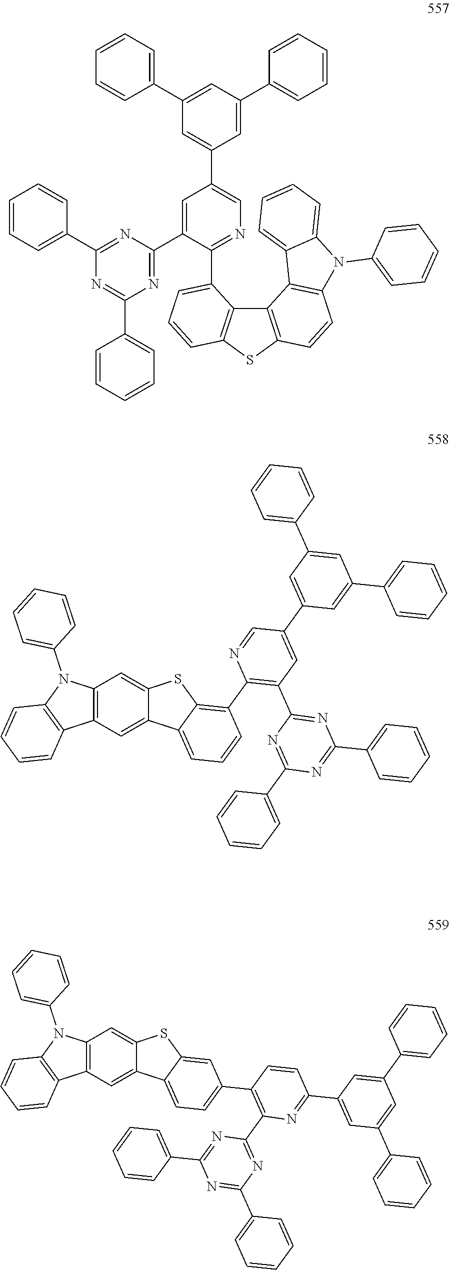 [SMILES: c1ccc(-c2cc(-c3ccccc3)cc(-c3cnc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4cc6c(cc45)c4ccccc4n6-c4ccccc4)c(-c4nc(-c5ccccc5)nc(-c5ccccc5)n4)n3)c2)cc1]

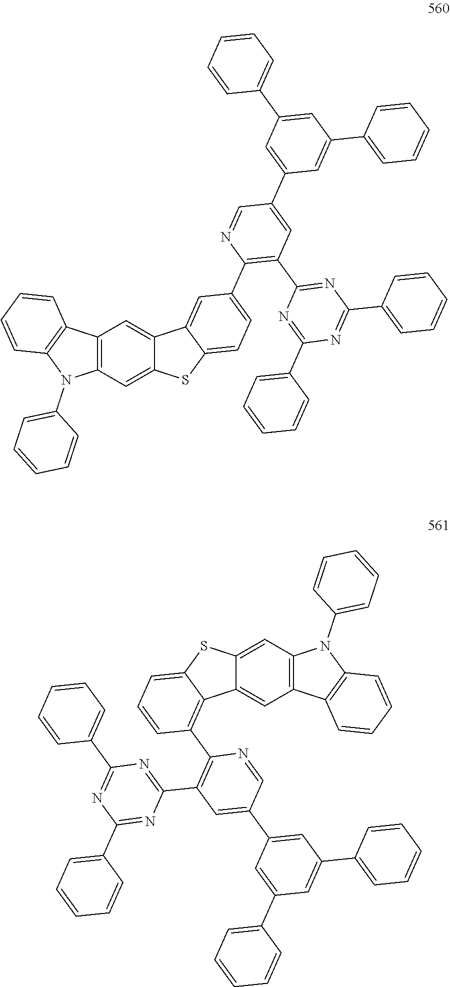 [SMILES: c1ccc(-c2cc(-c3ccccc3)cc(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3cnc(-c4cccc5sc6cc7c(cc6c45)c4ccccc4n7-c4ccccc4)c(-c4nc(-c5ccccc5)nc(-c5ccccc5)n4)c3)c2)cc1]

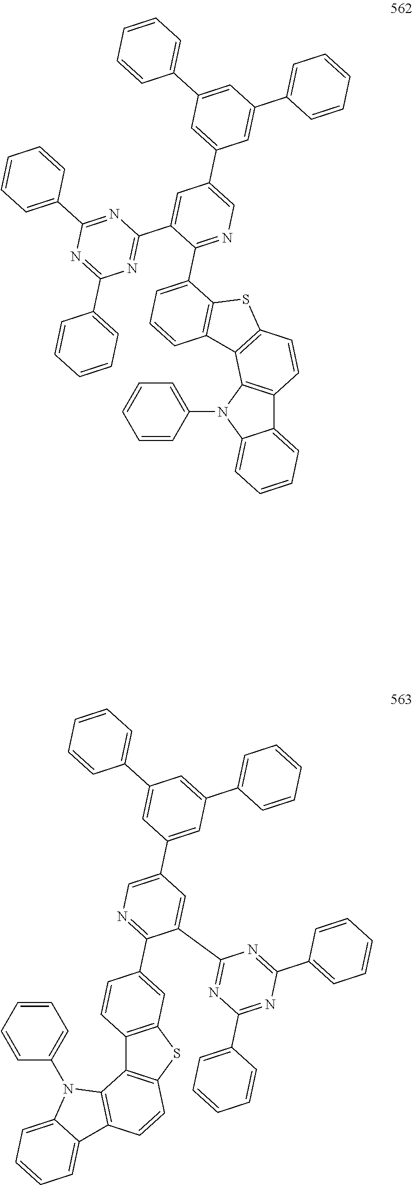 [SMILES: c1ccc(-c2cc(-c3ccccc3)cc(-c3cnc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4ccc6c7ccccc7n(-c7ccccc7)c6c45)c(-c4nc(-c5ccccc5)nc(-c5ccccc5)n4)c3)c2)cc1]

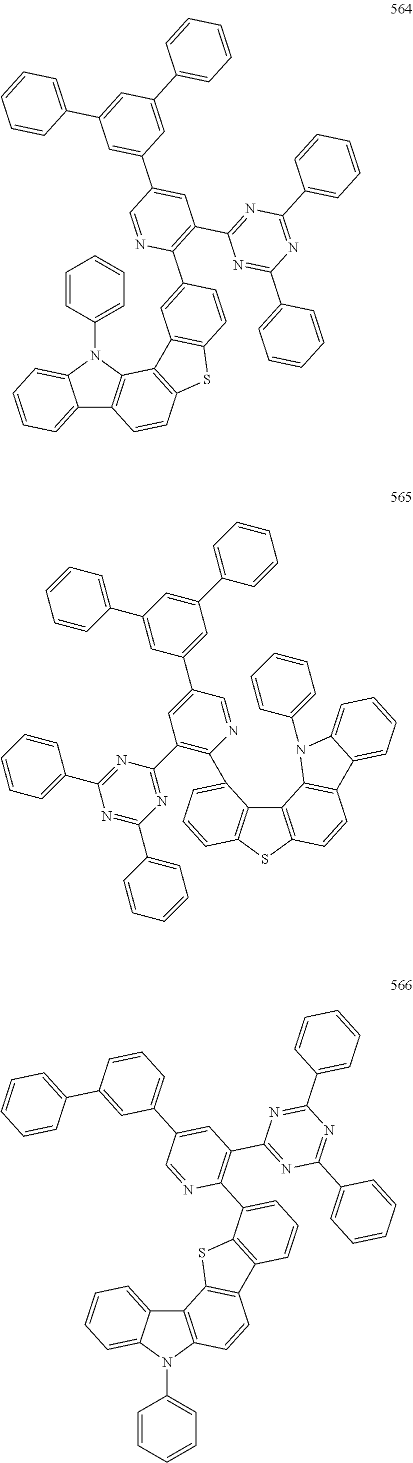 [SMILES: c1ccc(-c2cc(-c3ccccc3)cc(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2cccc(-c3cnc(-c4cccc5c4sc4c5ccc5c4c4ccccc4n5-c4ccccc4)c(-c4nc(-c5ccccc5)nc(-c5ccccc5)n4)c3)c2)cc1]

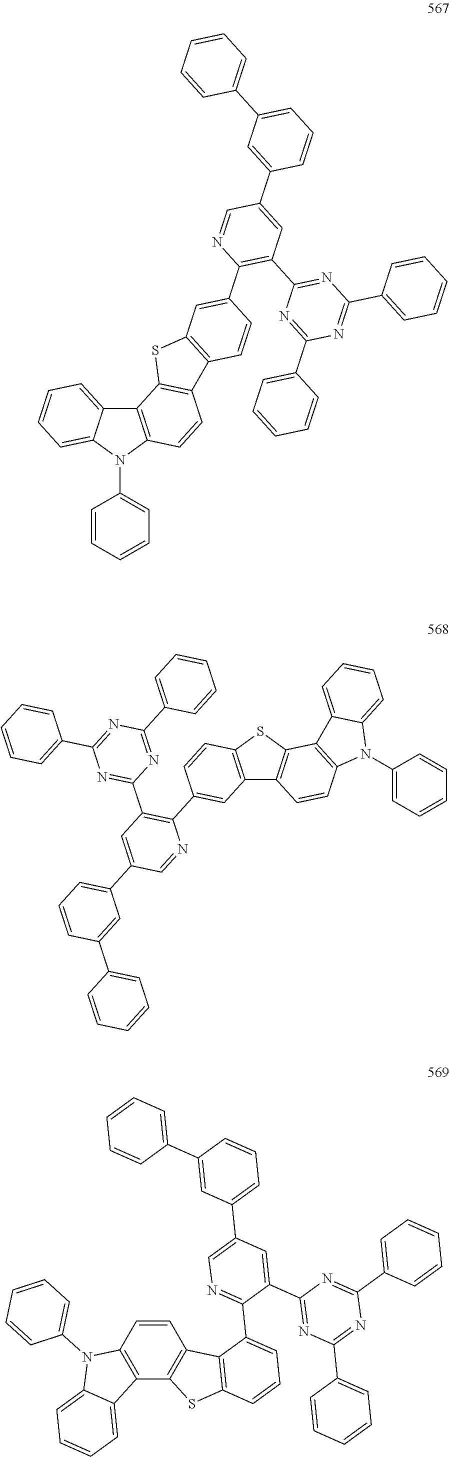 [SMILES: c1ccc(-c2cccc(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1 ; c1ccc(-c2cccc(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3cnc(-c4cccc5sc6c(ccc7c6c6ccccc6n7-c6ccccc6)c45)c(-c4nc(-c5ccccc5)nc(-c5ccccc5)n4)c3)c2)cc1]

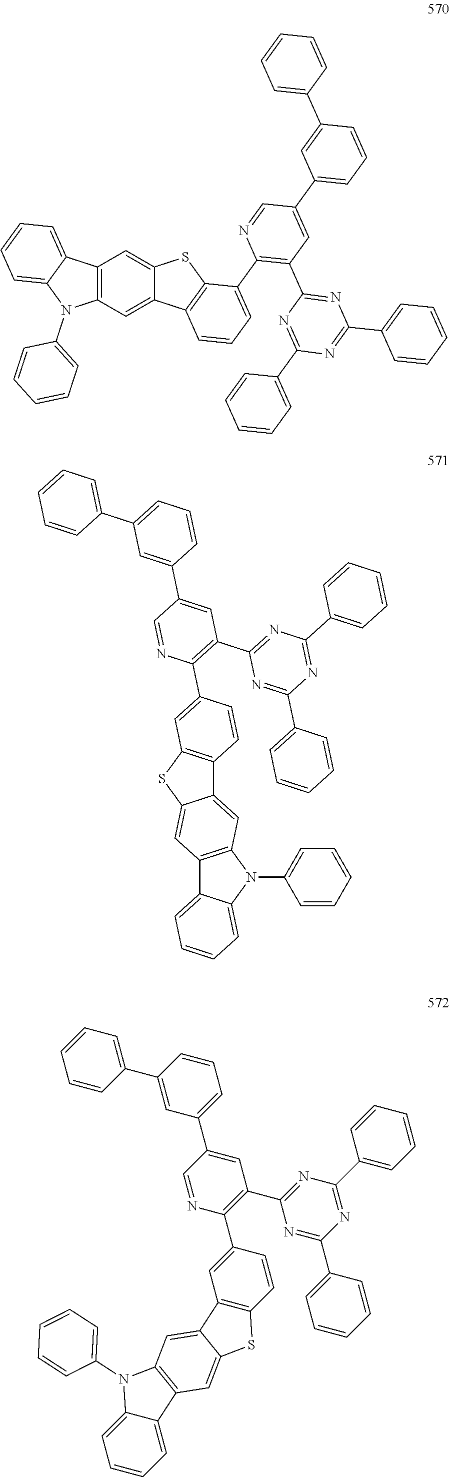 [SMILES: c1ccc(-c2cccc(-c3cnc(-c4cccc5c4sc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1]

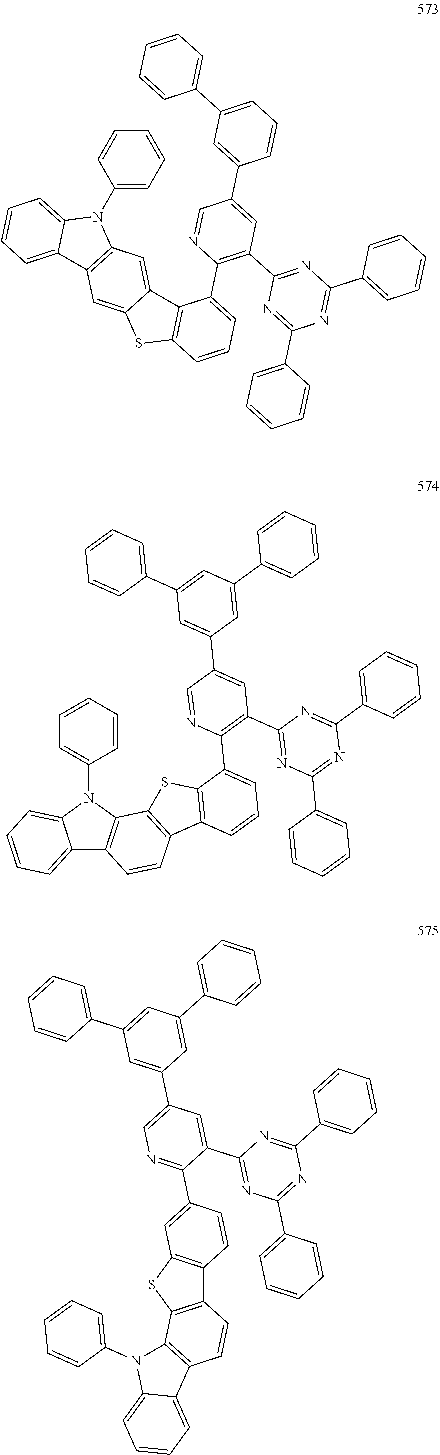 [SMILES: c1ccc(-c2cccc(-c3cnc(-c4cccc5sc6cc7c8ccccc8n(-c8ccccc8)c7cc6c45)c(-c4nc(-c5ccccc5)nc(-c5ccccc5)n4)c3)c2)cc1 ; c1ccc(-c2cc(-c3ccccc3)cc(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1]

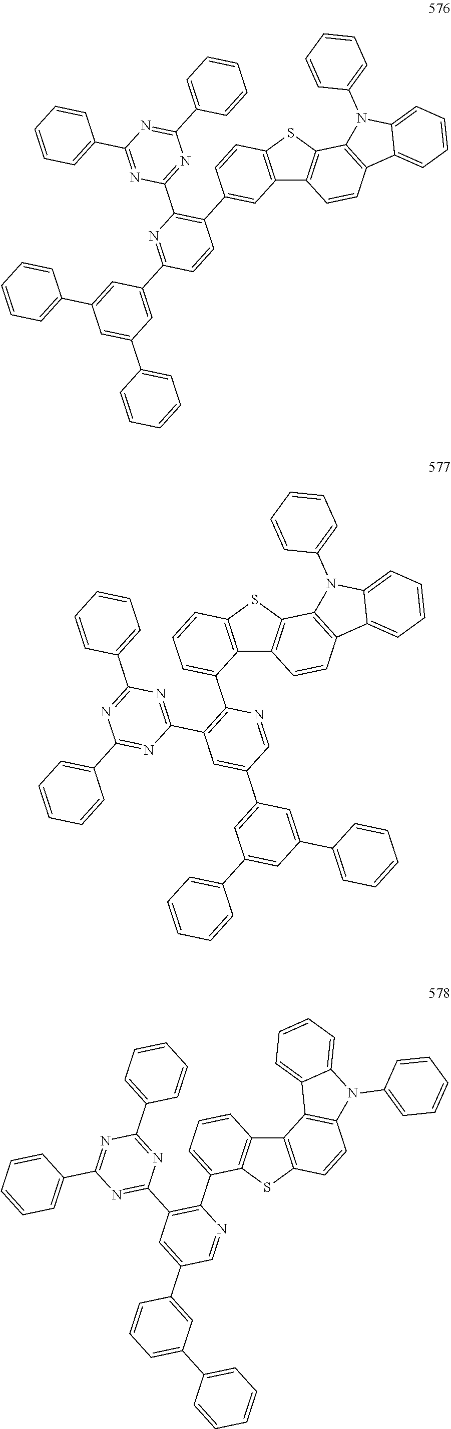 [SMILES: c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)n3)c2)cc1 ; c1ccc(-c2cc(-c3ccccc3)cc(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3cnc(-c4cccc5c4sc4ccc6c(c7ccccc7n6-c6ccccc6)c45)c(-c4nc(-c5ccccc5)nc(-c5ccccc5)n4)c3)c2)cc1]

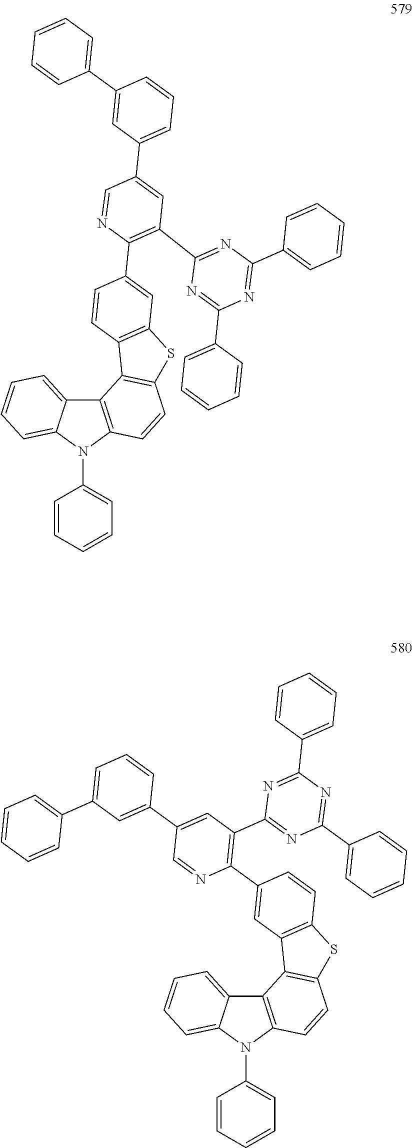 [SMILES: c1ccc(-c2cccc(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1]

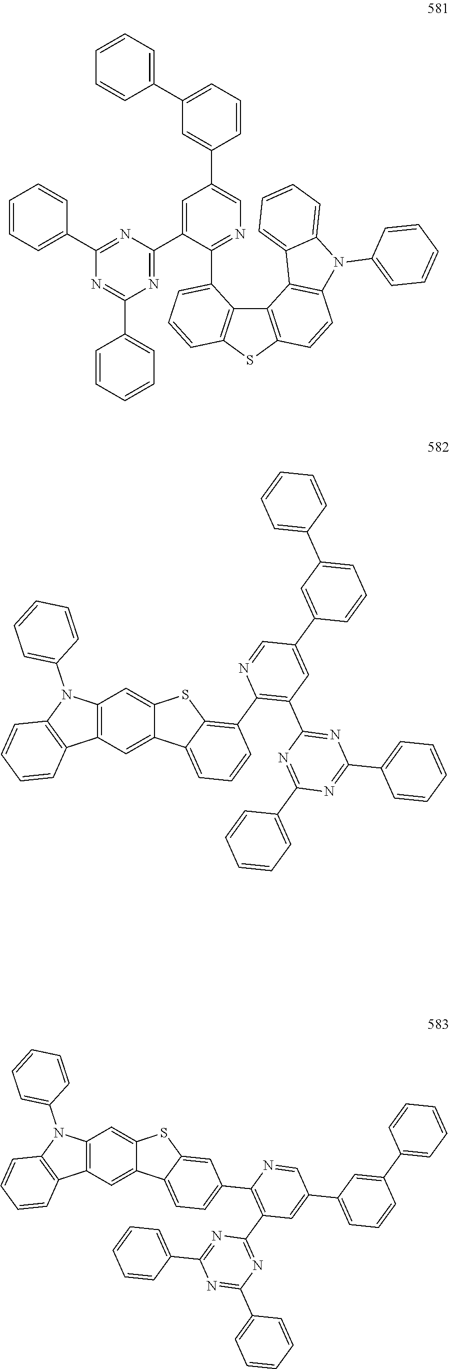 [SMILES: c1ccc(-c2cccc(-c3cnc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3cnc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1]

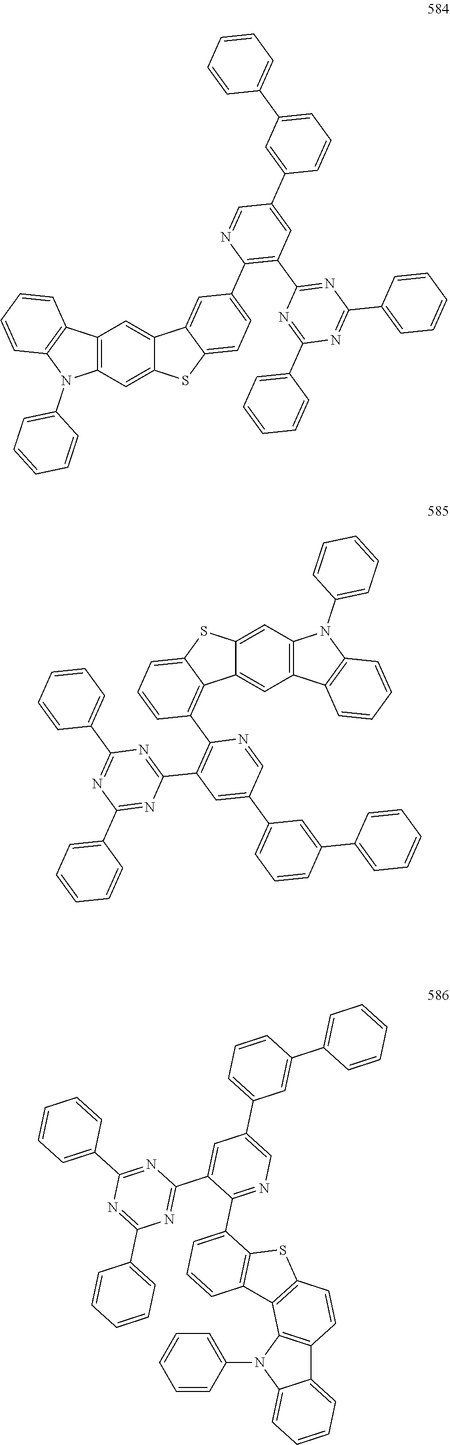 [SMILES: c1ccc(-c2cccc(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3cnc(-c4cccc5sc6cc7c(cc6c45)c4ccccc4n7-c4ccccc4)c(-c4nc(-c5ccccc5)nc(-c5ccccc5)n4)c3)c2)cc1 ; c1ccc(-c2cccc(-c3cnc(-c4cccc5c4sc4ccc6c7ccccc7n(-c7ccccc7)c6c45)c(-c4nc(-c5ccccc5)nc(-c5ccccc5)n4)c3)c2)cc1]

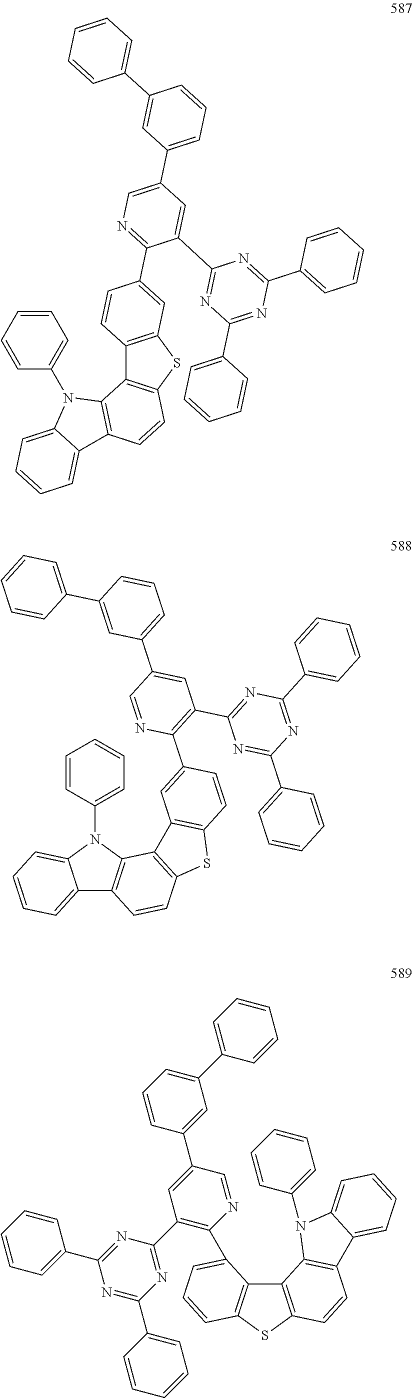 [SMILES: c1ccc(-c2cccc(-c3cnc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3cnc(-c4cccc5sc6ccc7c8ccccc8n(-c8ccccc8)c7c6c45)c(-c4nc(-c5ccccc5)nc(-c5ccccc5)n4)c3)c2)cc1]

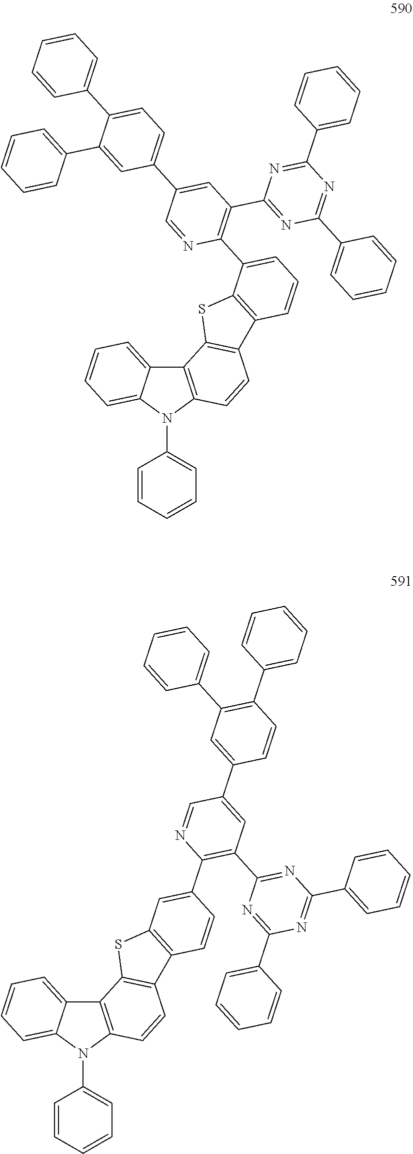 [SMILES: c1ccc(-c2nc(-c3ccccc3)nc(-c3cc(-c4ccc(-c5ccccc5)c(-c5ccccc5)c4)cnc3-c3cccc4c3sc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)cnc3-c3ccc4c(c3)sc3c4ccc4c3c3ccccc3n4-c3ccccc3)n2)cc1]

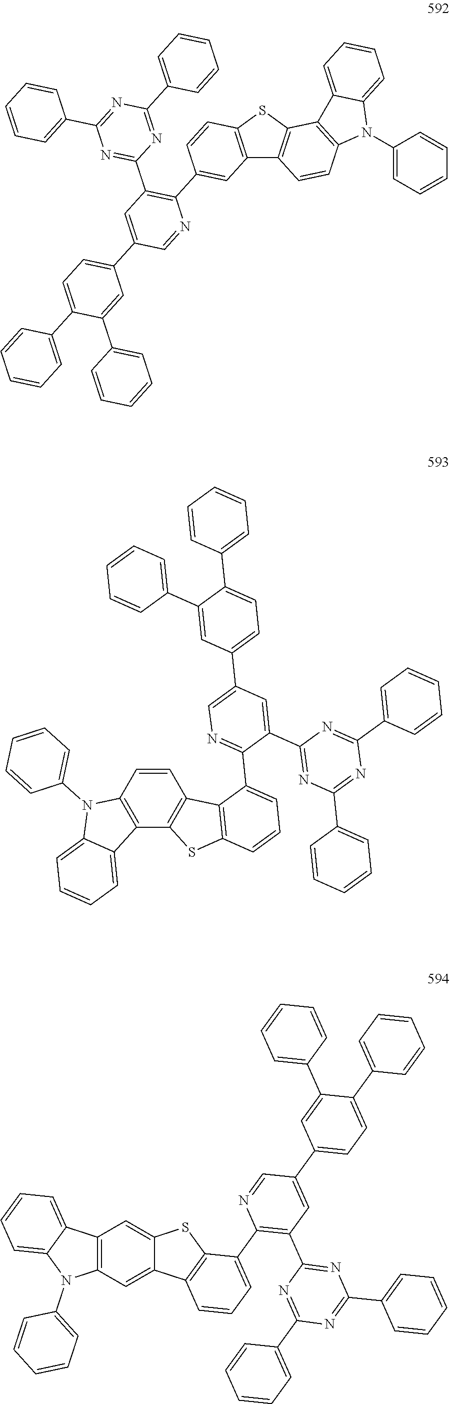 [SMILES: c1ccc(-c2nc(-c3ccccc3)nc(-c3cc(-c4ccc(-c5ccccc5)c(-c5ccccc5)c4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4sc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c6ccccc6n(-c6ccccc6)c5cc34)n2)cc1]

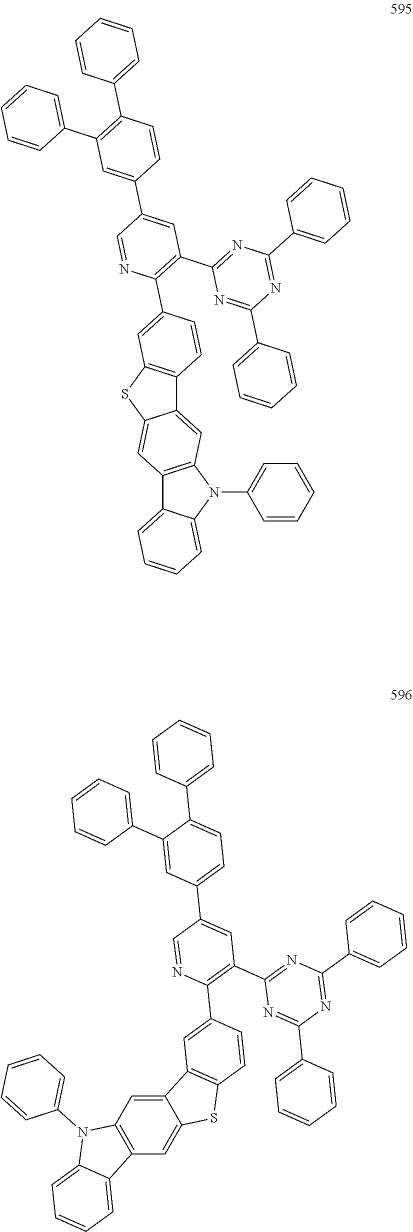 [SMILES: c1ccc(-c2nc(-c3ccccc3)nc(-c3cc(-c4ccc(-c5ccccc5)c(-c5ccccc5)c4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4sc5cc6c7ccccc7n(-c7ccccc7)c6cc5c4c3)n2)cc1]

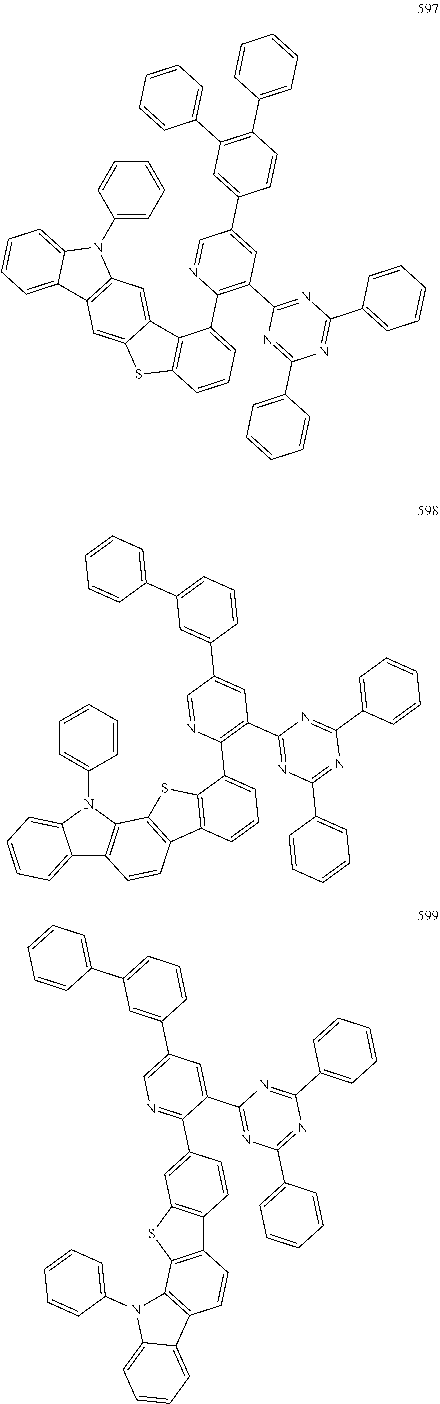

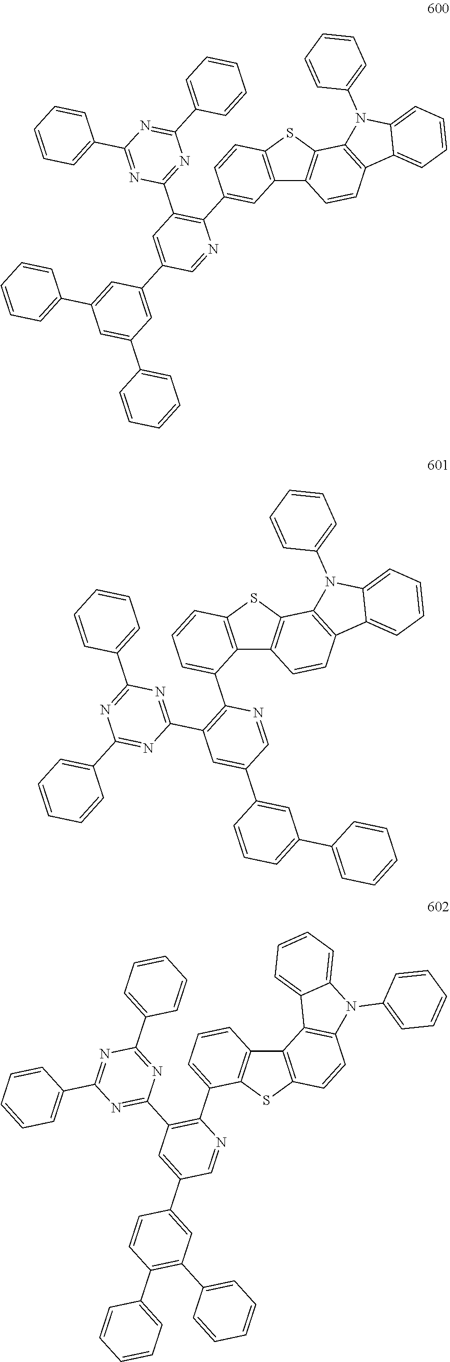 [SMILES: c1ccc(-c2cc(-c3ccccc3)cc(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)cnc3-c3cccc4c3sc3ccc5c(c6ccccc6n5-c5ccccc5)c34)n2)cc1]

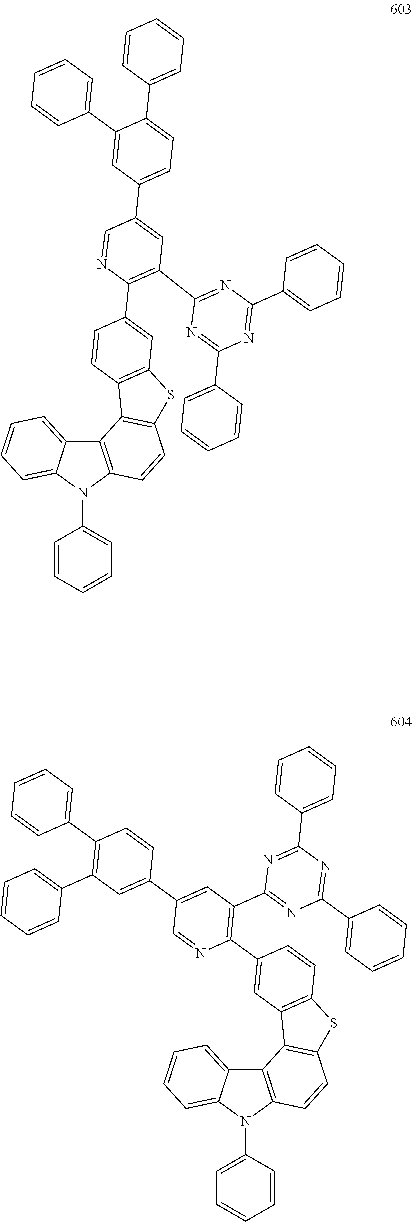 [SMILES: c1ccc(-c2nc(-c3ccccc3)nc(-c3cc(-c4ccc(-c5ccccc5)c(-c5ccccc5)c4)cnc3-c3ccc4c(c3)sc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4sc5ccc6c(c7ccccc7n6-c6ccccc6)c5c4c3)n2)cc1]

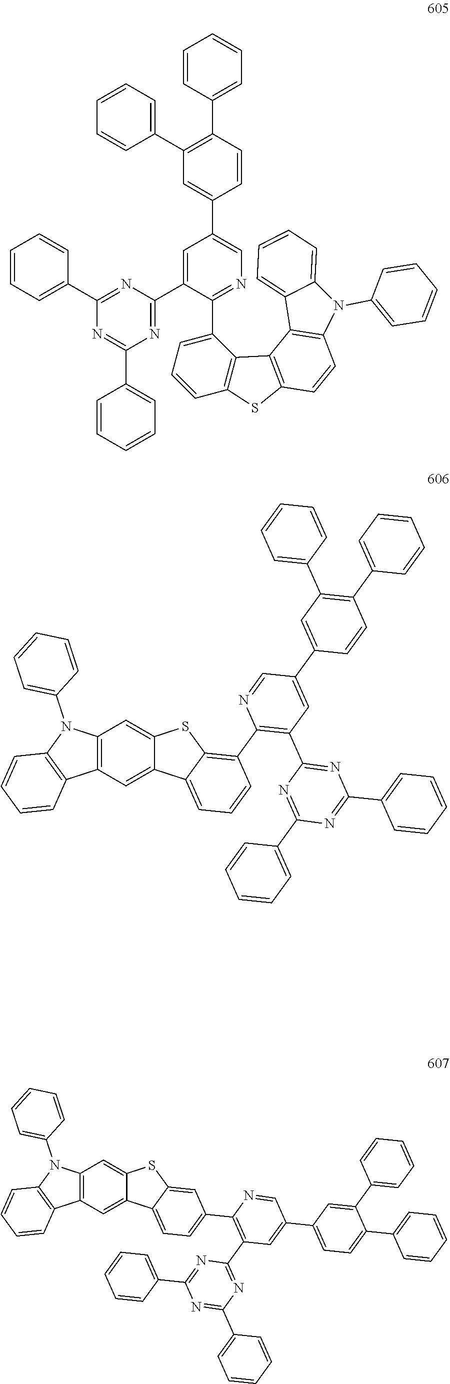 [SMILES: c1ccc(-c2nc(-c3ccccc3)nc(-c3cc(-c4ccc(-c5ccccc5)c(-c5ccccc5)c4)cnc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4c(c3)sc3cc5c(cc34)c3ccccc3n5-c3ccccc3)n2)cc1]

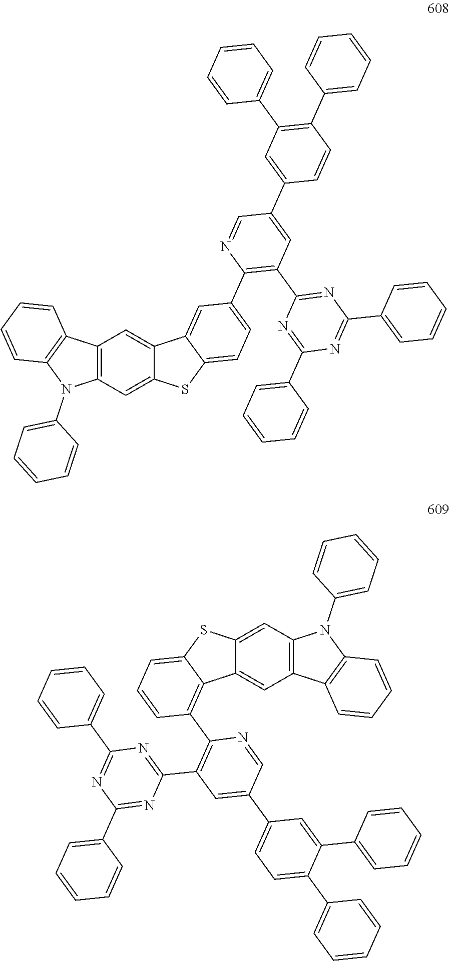 [SMILES: c1ccc(-c2nc(-c3ccccc3)nc(-c3cc(-c4ccc(-c5ccccc5)c(-c5ccccc5)c4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4sc5cc6c(cc5c34)c3ccccc3n6-c3ccccc3)n2)cc1]

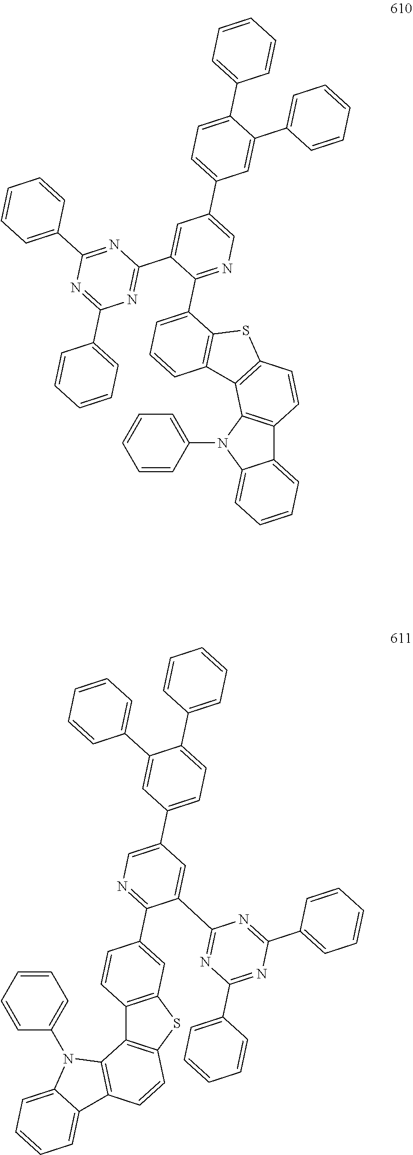 [SMILES: c1ccc(-c2nc(-c3ccccc3)nc(-c3cc(-c4ccc(-c5ccccc5)c(-c5ccccc5)c4)cnc3-c3cccc4c3sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3ccc5c6ccccc6n(-c6ccccc6)c5c34)n2)cc1]

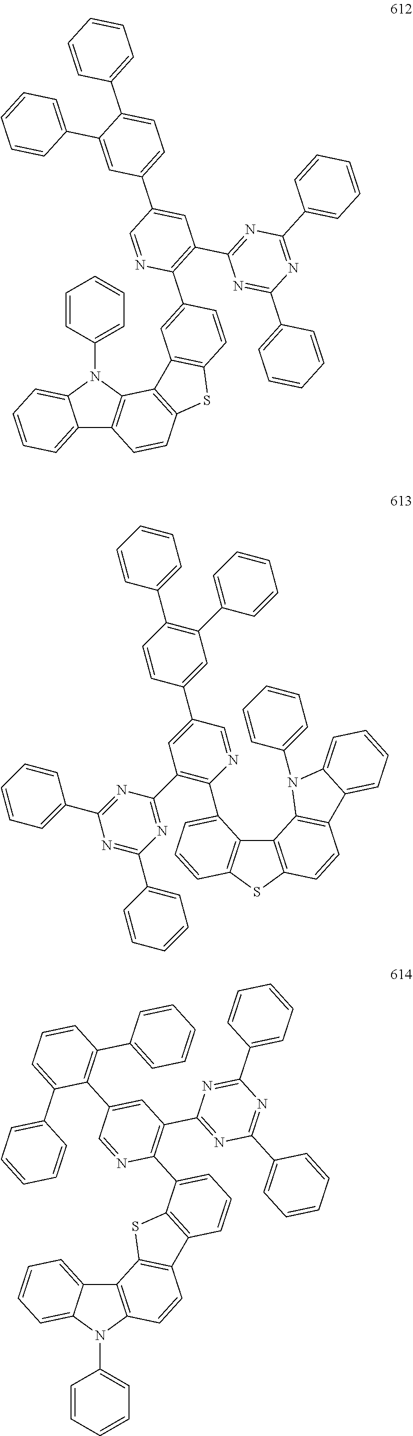 [SMILES: c1ccc(-c2nc(-c3ccccc3)nc(-c3cc(-c4ccc(-c5ccccc5)c(-c5ccccc5)c4)cnc3-c3ccc4sc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)cnc3-c3cccc4sc5ccc6c7ccccc7n(-c7ccccc7)c6c5c34)n2)cc1 ; c1ccc(-c2nc(-c3ccccc3)nc(-c3cc(-c4c(-c5ccccc5)cccc4-c4ccccc4)cnc3-c3cccc4c3sc3c4ccc4c3c3ccccc3n4-c3ccccc3)n2)cc1]

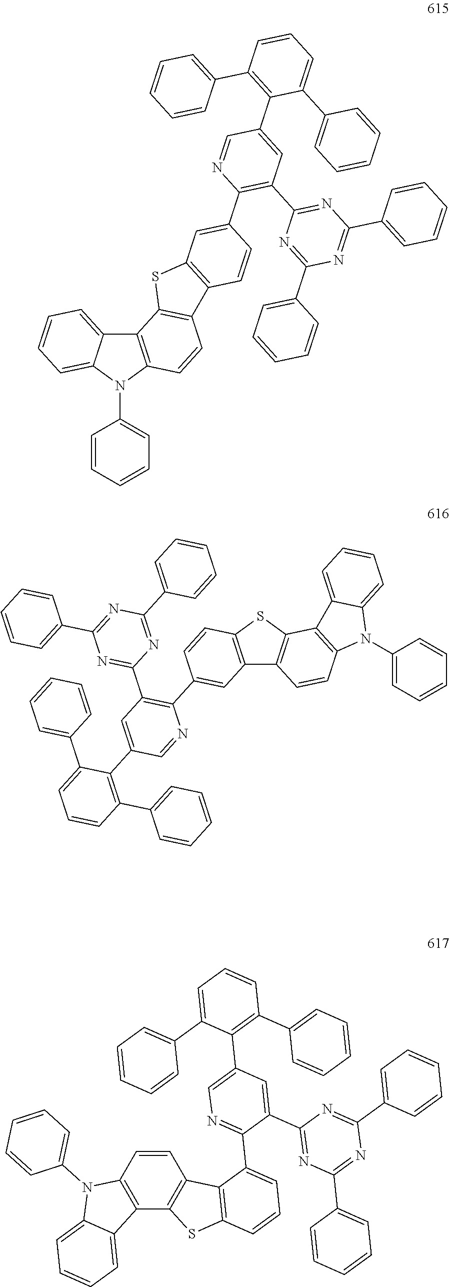 [SMILES: c1ccc(-c2nc(-c3ccccc3)nc(-c3cc(-c4c(-c5ccccc5)cccc4-c4ccccc4)cnc3-c3ccc4c(c3)sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4sc5c(ccc6c5c5ccccc5n6-c5ccccc5)c34)n2)cc1]

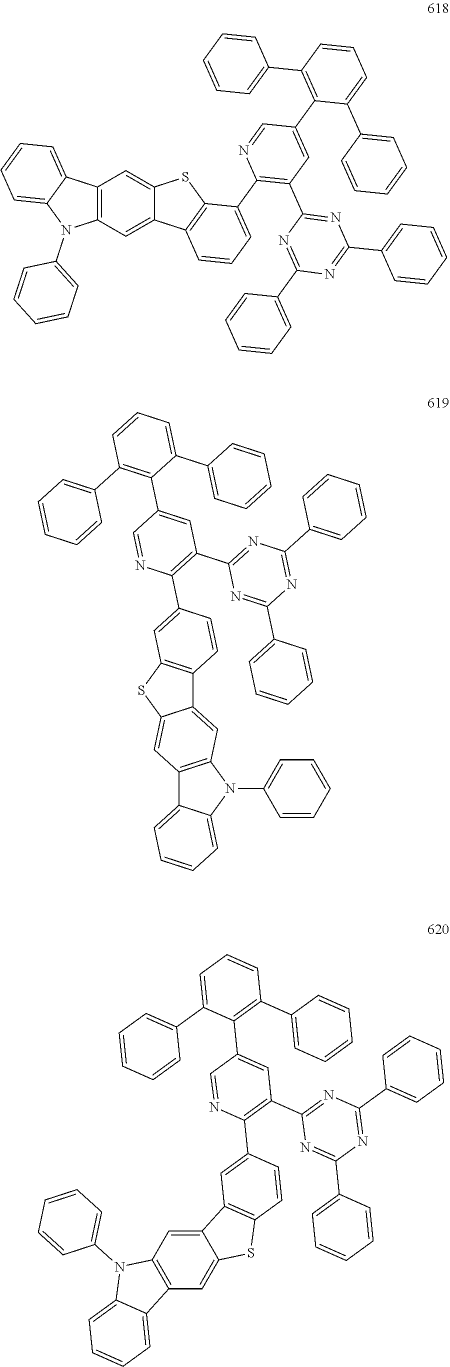 [SMILES: c1ccc(-c2nc(-c3ccccc3)nc(-c3cc(-c4c(-c5ccccc5)cccc4-c4ccccc4)cnc3-c3cccc4c3sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4sc5cc6c7ccccc7n(-c7ccccc7)c6cc5c4c3)n2)cc1]

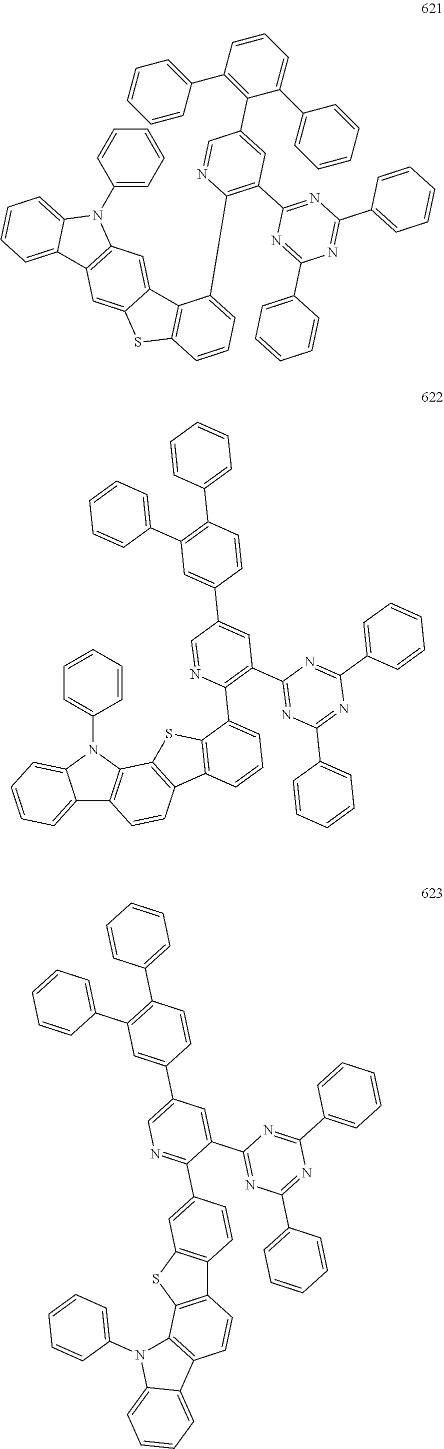 [SMILES: c1ccc(-c2nc(-c3ccccc3)nc(-c3cc(-c4c(-c5ccccc5)cccc4-c4ccccc4)cnc3-c3cccc4sc5cc6c7ccccc7n(-c7ccccc7)c6cc5c34)n2)cc1 ; c1ccc(-c2nc(-c3ccccc3)nc(-c3cc(-c4ccc(-c5ccccc5)c(-c5ccccc5)c4)cnc3-c3cccc4c3sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)sc3c4ccc4c5ccccc5n(-c5ccccc5)c43)n2)cc1]

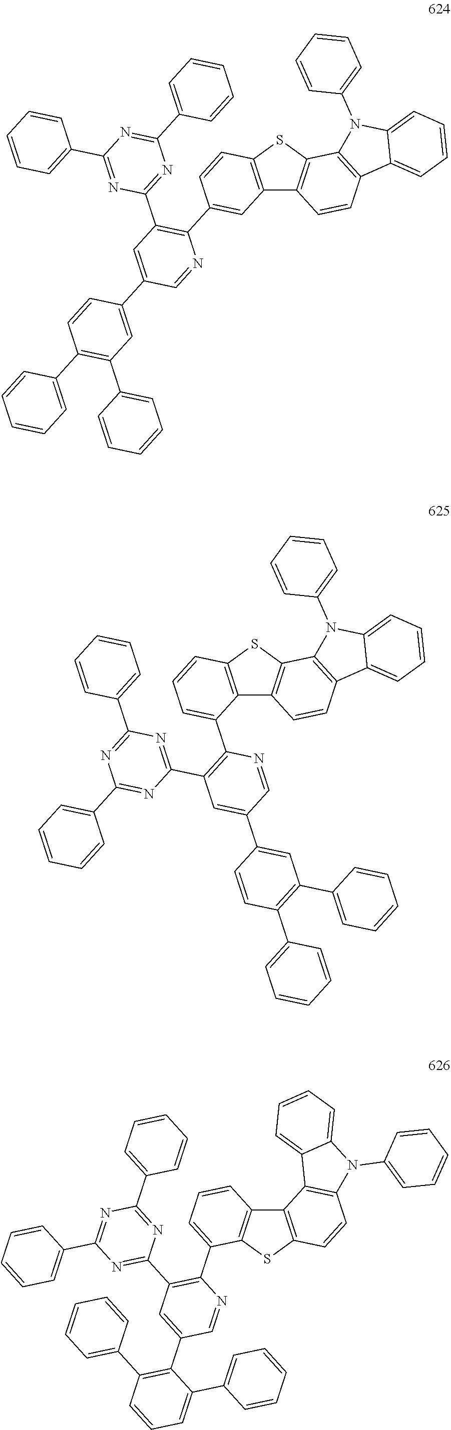 [SMILES: c1ccc(-c2nc(-c3ccccc3)nc(-c3cc(-c4ccc(-c5ccccc5)c(-c5ccccc5)c4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)cnc3-c3cccc4sc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)cnc3-c3cccc4c3sc3ccc5c(c6ccccc6n5-c5ccccc5)c34)n2)cc1]

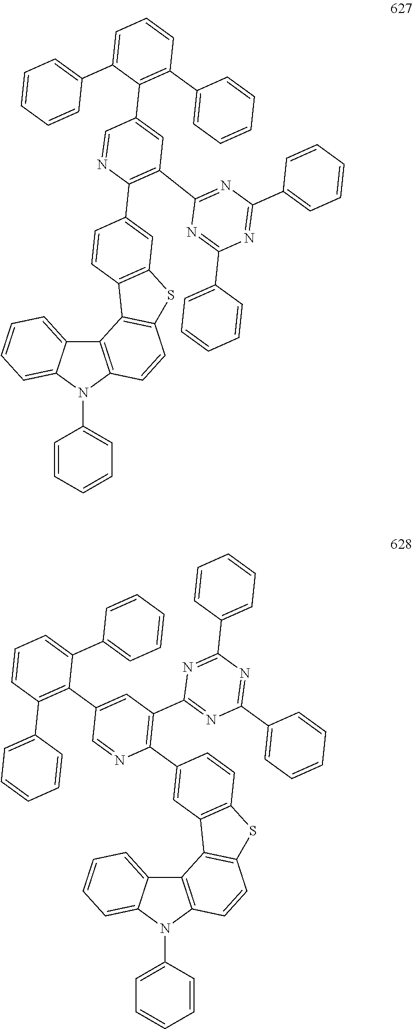 [SMILES: c1ccc(-c2nc(-c3ccccc3)nc(-c3cc(-c4c(-c5ccccc5)cccc4-c4ccccc4)cnc3-c3ccc4c(c3)sc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)cnc3-c3ccc4sc5ccc6c(c7ccccc7n6-c6ccccc6)c5c4c3)n2)cc1]

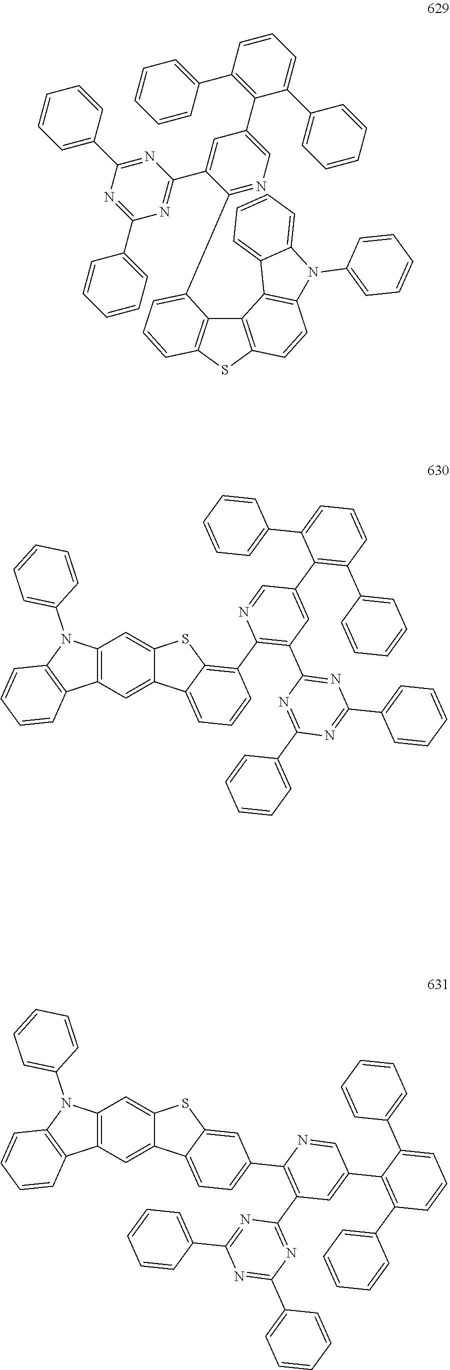 [SMILES: c1ccc(-c2nc(-c3ccccc3)nc(-c3cc(-c4c(-c5ccccc5)cccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)cnc3-c3cccc4c3sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)sc3cc5c(cc34)c3ccccc3n5-c3ccccc3)n2)cc1]

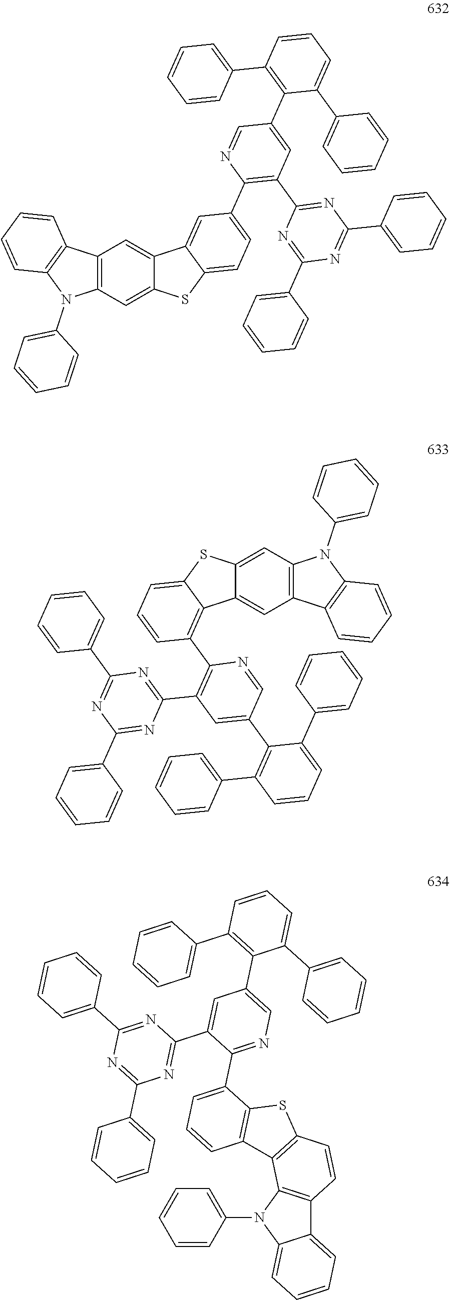 [SMILES: c1ccc(-c2nc(-c3ccccc3)nc(-c3cc(-c4c(-c5ccccc5)cccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4sc5cc6c(cc5c34)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4c3sc3ccc5c6ccccc6n(-c6ccccc6)c5c34)n2)cc1]

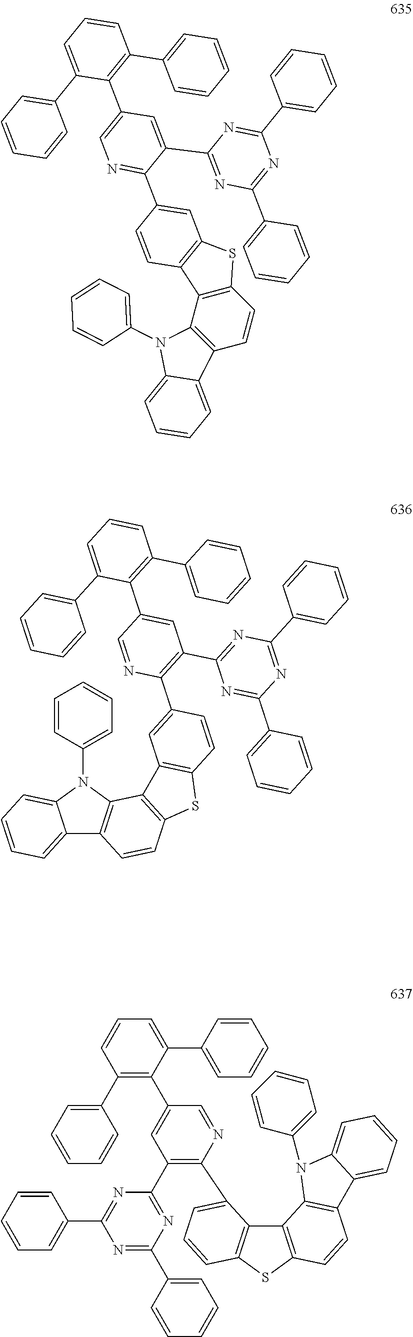 [SMILES: c1ccc(-c2nc(-c3ccccc3)nc(-c3cc(-c4c(-c5ccccc5)cccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4sc5ccc6c7ccccc7n(-c7ccccc7)c6c5c4c3)n2)cc1 ; c1ccc(-c2nc(-c3ccccc3)nc(-c3cc(-c4c(-c5ccccc5)cccc4-c4ccccc4)cnc3-c3cccc4sc5ccc6c7ccccc7n(-c7ccccc7)c6c5c34)n2)cc1]

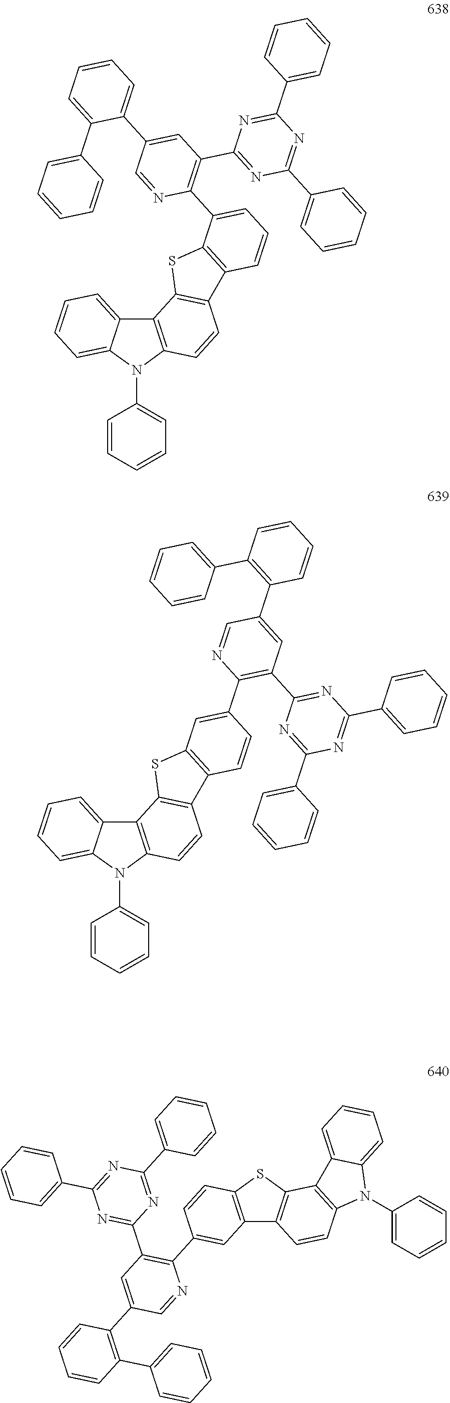 [SMILES: c1ccc(-c2nc(-c3ccccc3)nc(-c3cc(-c4ccccc4-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4c(c3)sc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4sc5c(ccc6c5c5ccccc5n6-c5ccccc5)c4c3)n2)cc1]

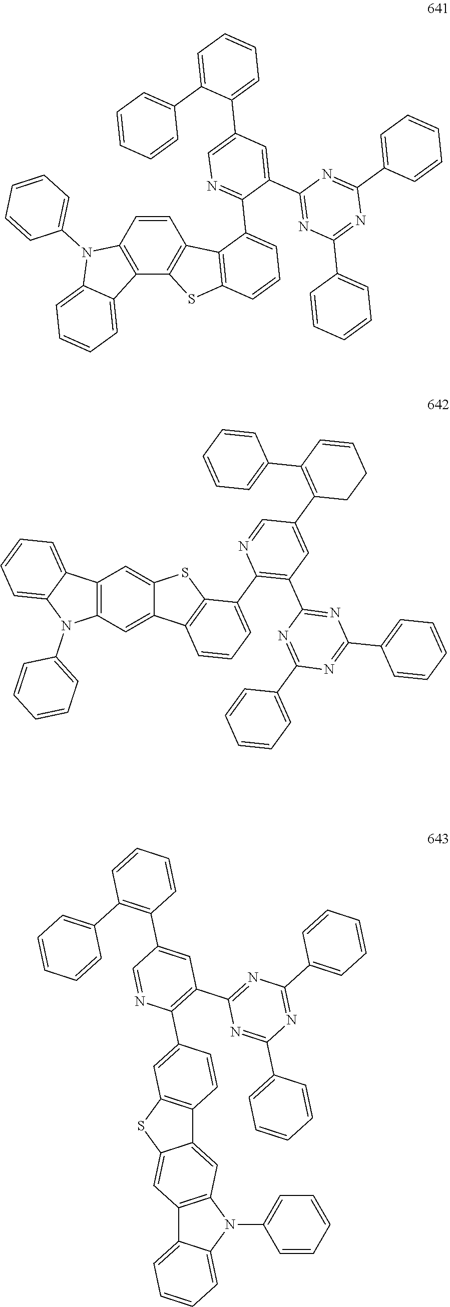 [SMILES: c1ccc(-c2nc(-c3ccccc3)nc(-c3cc(-c4ccccc4-c4ccccc4)cnc3-c3cccc4sc5c(ccc6c5c5ccccc5n6-c5ccccc5)c34)n2)cc1 ; C1=CC(c2ccccc2)=C(c2cnc(-c3cccc4c3sc3cc5c6ccccc6n(-c6ccccc6)c5cc34)c(-c3nc(-c4ccccc4)nc(-c4ccccc4)n3)c2)CC1 ; c1ccc(-c2nc(-c3ccccc3)nc(-c3cc(-c4ccccc4-c4ccccc4)cnc3-c3ccc4c(c3)sc3cc5c6ccccc6n(-c6ccccc6)c5cc34)n2)cc1]

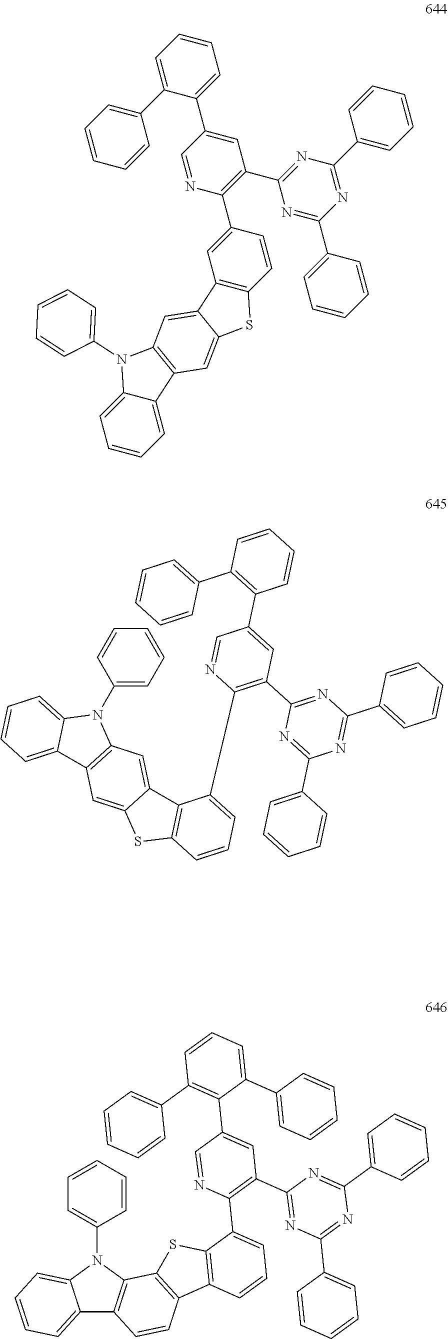 [SMILES: c1ccc(-c2nc(-c3ccccc3)nc(-c3cc(-c4ccccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4sc5cc6c7ccccc7n(-c7ccccc7)c6cc5c34)n2)cc1 ; c1ccc(-c2nc(-c3ccccc3)nc(-c3cc(-c4c(-c5ccccc5)cccc4-c4ccccc4)cnc3-c3cccc4c3sc3c4ccc4c5ccccc5n(-c5ccccc5)c43)n2)cc1]

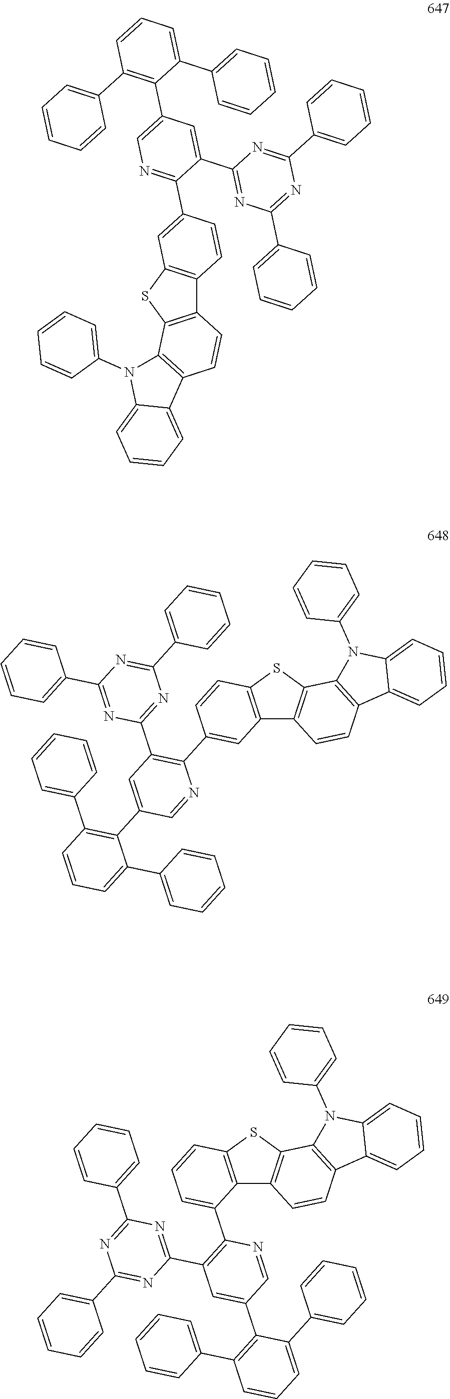 [SMILES: c1ccc(-c2nc(-c3ccccc3)nc(-c3cc(-c4c(-c5ccccc5)cccc4-c4ccccc4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4c(-c5ccccc5)cccc4-c4ccccc4)cnc3-c3cccc4sc5c(ccc6c7ccccc7n(-c7ccccc7)c65)c34)n2)cc1]

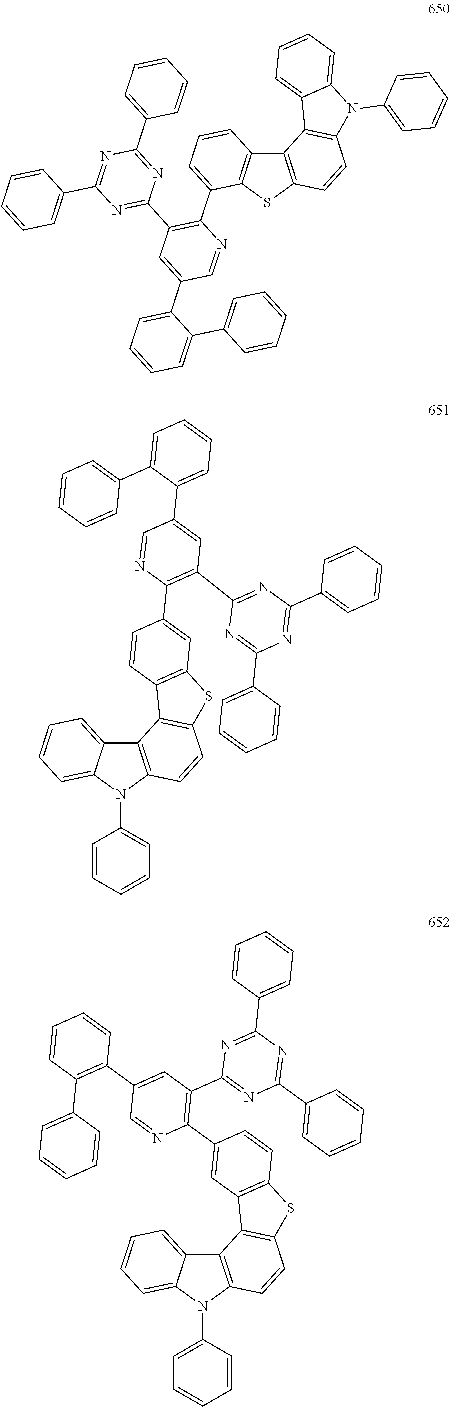 [SMILES: c1ccc(-c2nc(-c3ccccc3)nc(-c3cc(-c4ccccc4-c4ccccc4)cnc3-c3cccc4c3sc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4c(c3)sc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4sc5ccc6c(c7ccccc7n6-c6ccccc6)c5c4c3)n2)cc1]

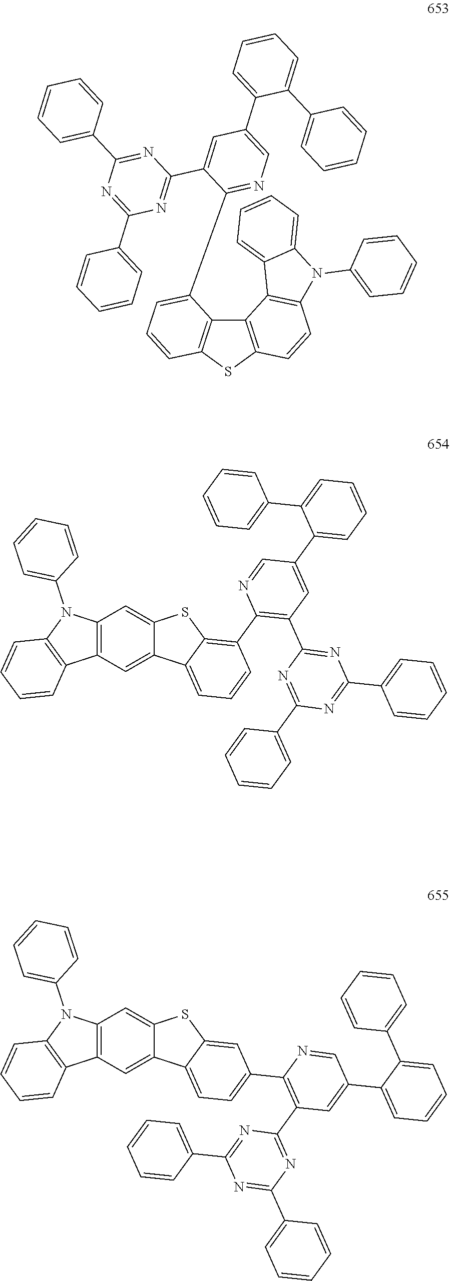 [SMILES: c1ccc(-c2nc(-c3ccccc3)nc(-c3cc(-c4ccccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4ccccc4-c4ccccc4)cnc3-c3cccc4c3sc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4c(c3)sc3cc5c(cc34)c3ccccc3n5-c3ccccc3)n2)cc1]

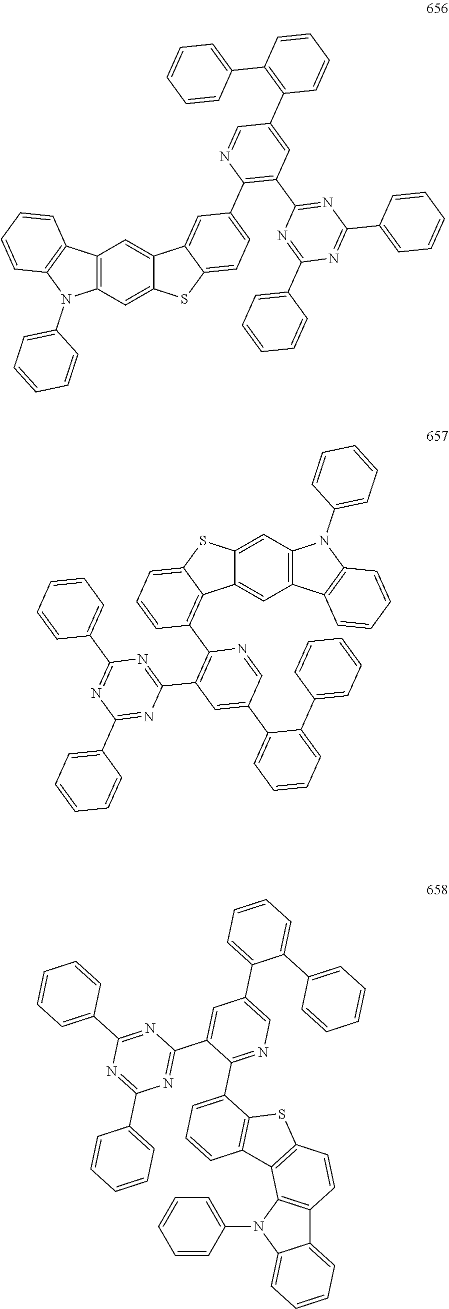 [SMILES: c1ccc(-c2nc(-c3ccccc3)nc(-c3cc(-c4ccccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4sc5cc6c(cc5c34)c3ccccc3n6-c3ccccc3)n2)cc1 ; c1ccc(-c2nc(-c3ccccc3)nc(-c3cc(-c4ccccc4-c4ccccc4)cnc3-c3cccc4c3sc3ccc5c6ccccc6n(-c6ccccc6)c5c34)n2)cc1]

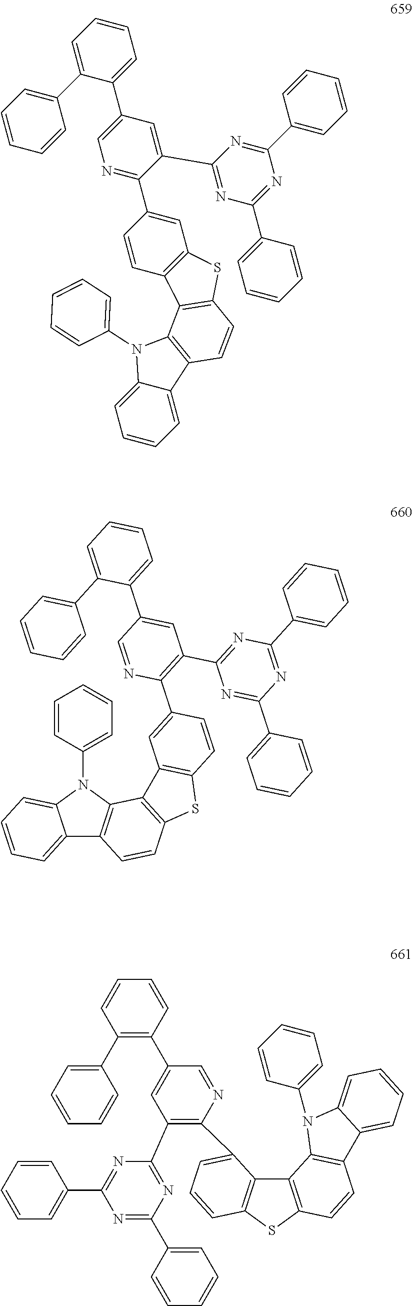 [SMILES: c1ccc(-c2nc(-c3ccccc3)nc(-c3cc(-c4ccccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4sc5ccc6c7ccccc7n(-c7ccccc7)c6c5c4c3)n2)cc1 ; c1ccc(-c2nc(-c3ccccc3)nc(-c3cc(-c4ccccc4-c4ccccc4)cnc3-c3cccc4sc5ccc6c7ccccc7n(-c7ccccc7)c6c5c34)n2)cc1]

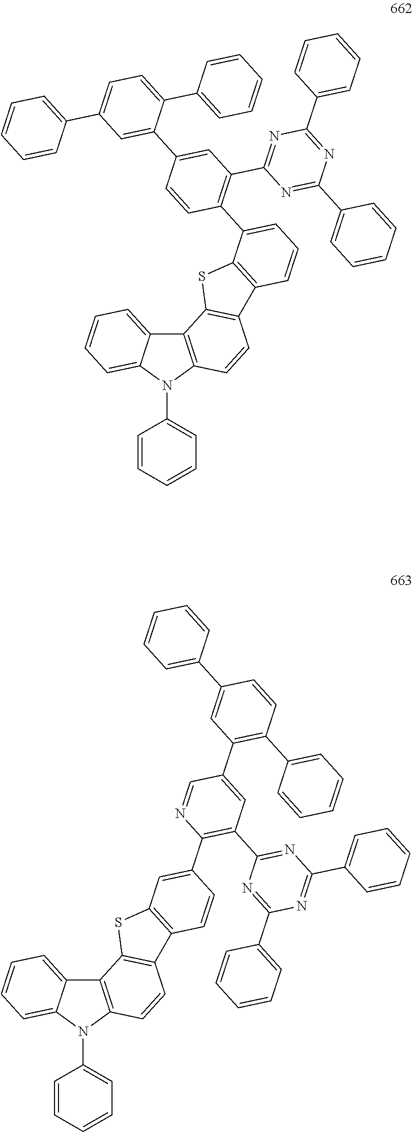 [SMILES: c1ccc(-c2ccc(-c3ccccc3)c(-c3ccc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1]

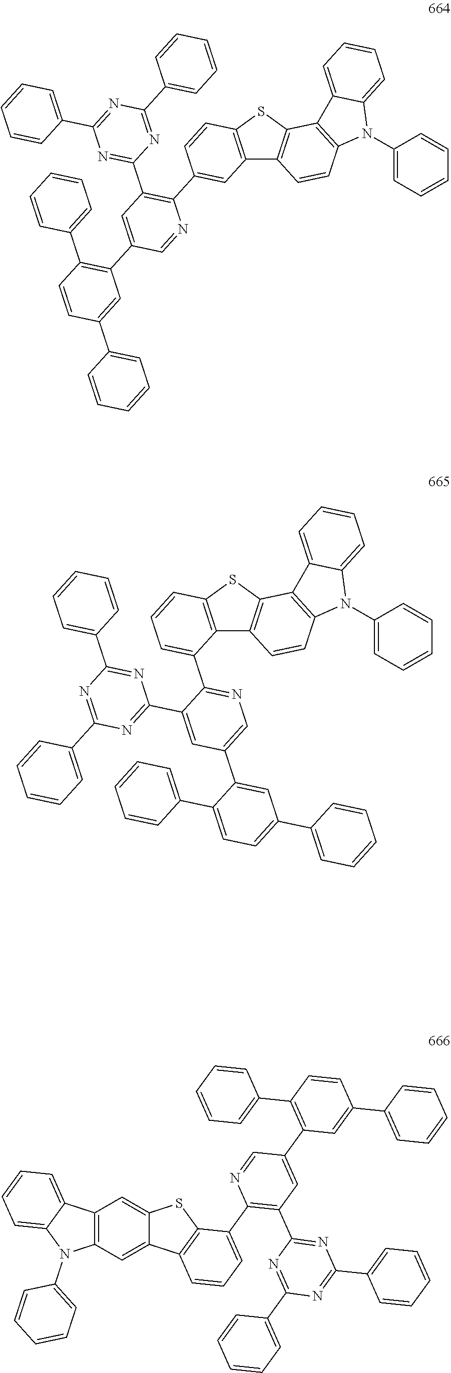 [SMILES: c1ccc(-c2ccc(-c3ccccc3)c(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4cc6c7ccccc7n(-c7ccccc7)c6cc45)c(-c4nc(-c5ccccc5)nc(-c5ccccc5)n4)c3)c2)cc1]

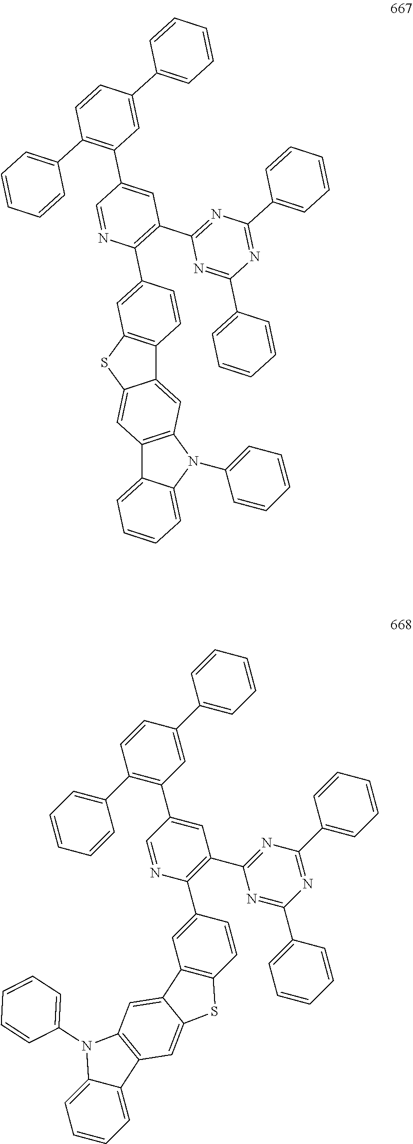 [SMILES: c1ccc(-c2ccc(-c3ccccc3)c(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1]

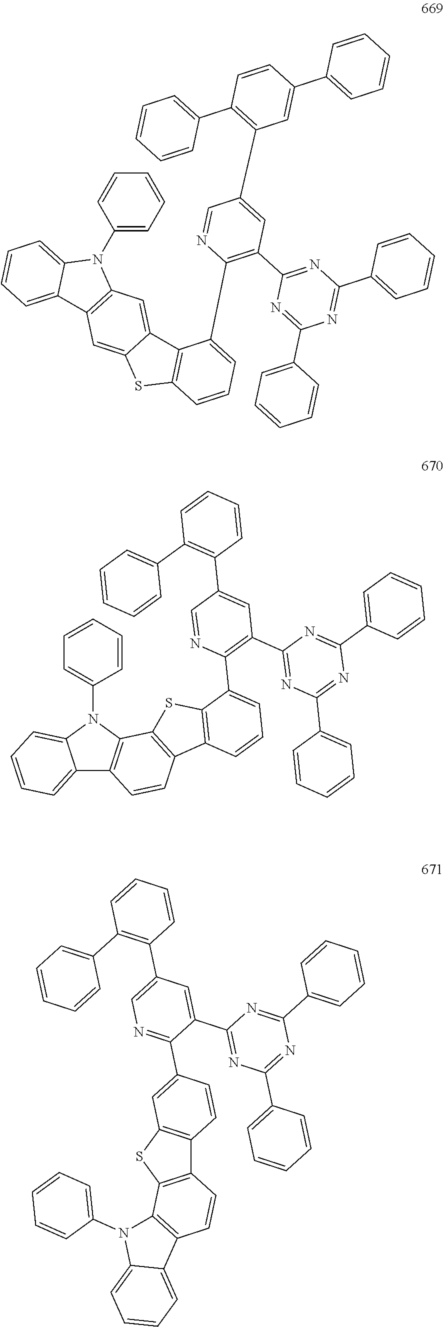 [SMILES: c1ccc(-c2ccc(-c3ccccc3)c(-c3cnc(-c4cccc5sc6cc7c8ccccc8n(-c8ccccc8)c7cc6c45)c(-c4nc(-c5ccccc5)nc(-c5ccccc5)n4)c3)c2)cc1 ; c1ccc(-c2nc(-c3ccccc3)nc(-c3cc(-c4ccccc4-c4ccccc4)cnc3-c3cccc4c3sc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)sc3c4ccc4c5ccccc5n(-c5ccccc5)c43)n2)cc1]

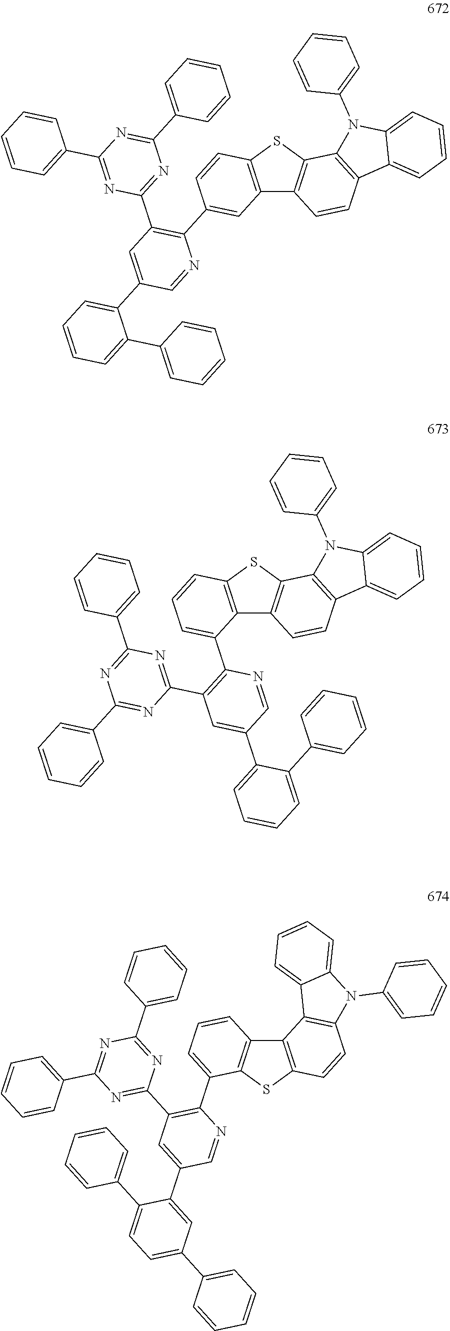 [SMILES: c1ccc(-c2nc(-c3ccccc3)nc(-c3cc(-c4ccccc4-c4ccccc4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)cnc3-c3cccc4sc5c(ccc6c7ccccc7n(-c7ccccc7)c65)c34)n2)cc1 ; c1ccc(-c2ccc(-c3ccccc3)c(-c3cnc(-c4cccc5c4sc4ccc6c(c7ccccc7n6-c6ccccc6)c45)c(-c4nc(-c5ccccc5)nc(-c5ccccc5)n4)c3)c2)cc1]

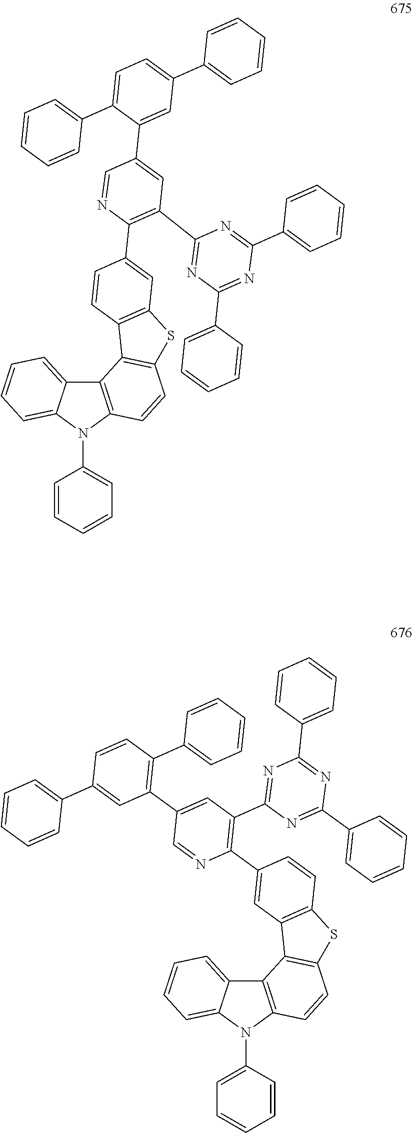 [SMILES: c1ccc(-c2ccc(-c3ccccc3)c(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1]

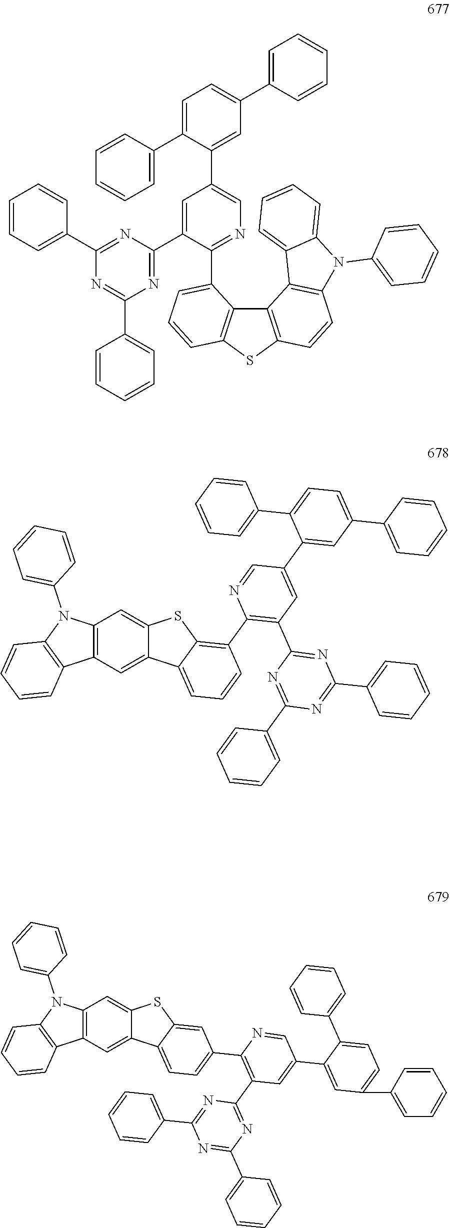 [SMILES: c1ccc(-c2ccc(-c3ccccc3)c(-c3cnc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1]

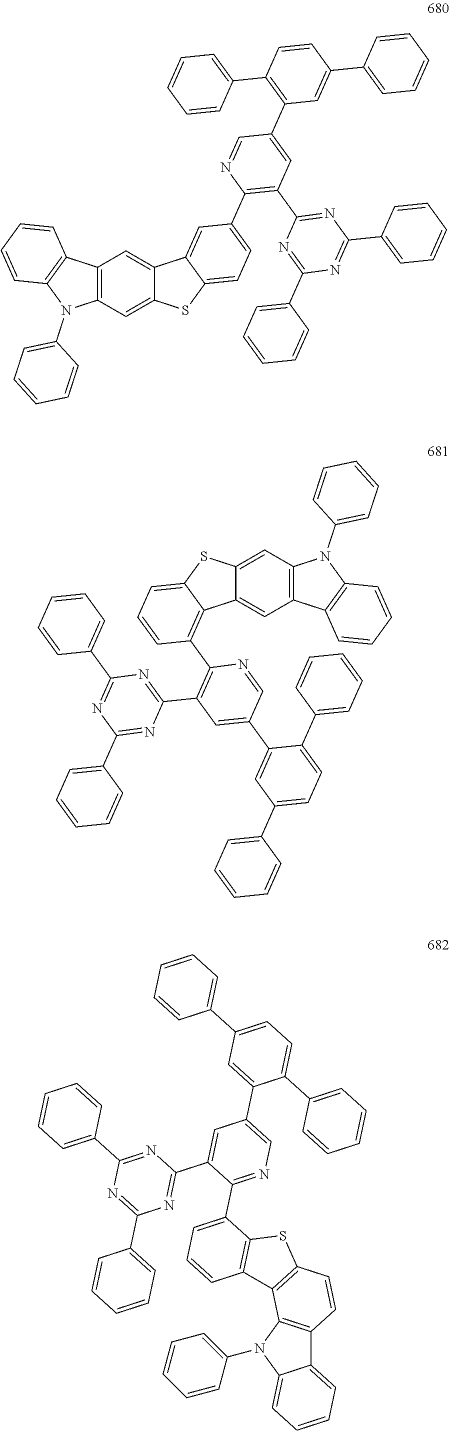 [SMILES: c1ccc(-c2ccc(-c3ccccc3)c(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5sc6cc7c(cc6c45)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5c4sc4ccc6c7ccccc7n(-c7ccccc7)c6c45)c(-c4nc(-c5ccccc5)nc(-c5ccccc5)n4)c3)c2)cc1]

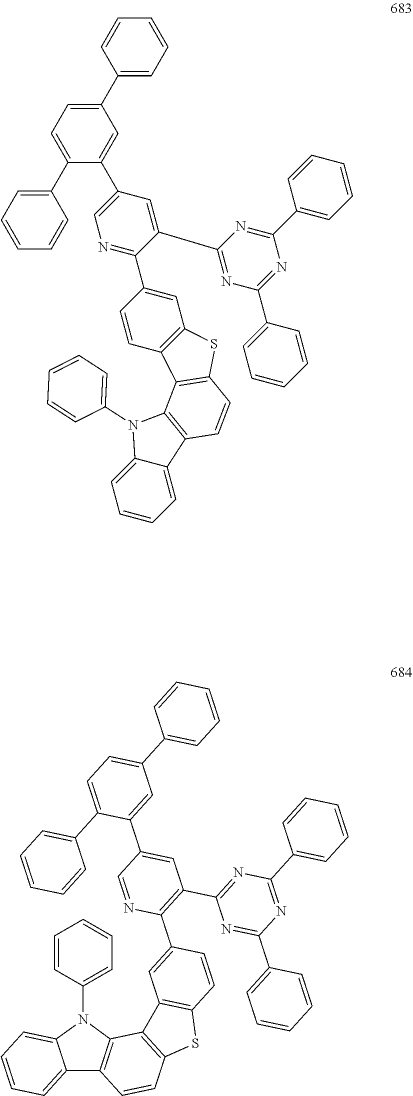 [SMILES: c1ccc(-c2ccc(-c3ccccc3)c(-c3cnc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1]

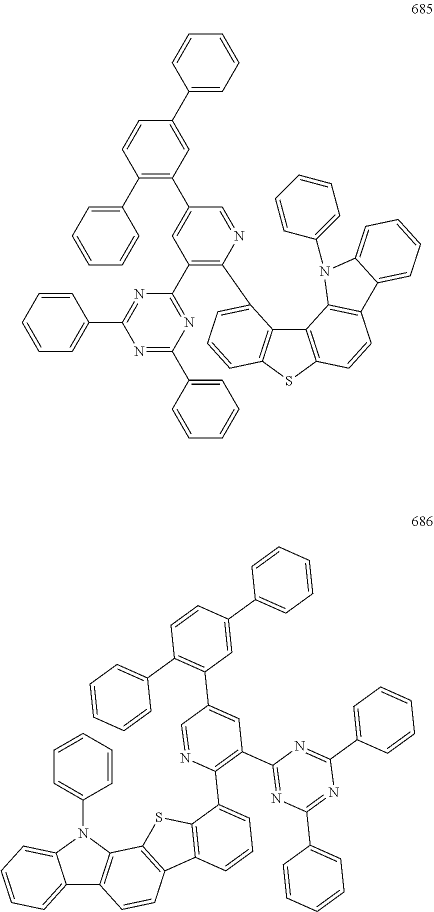 [SMILES: c1ccc(-c2ccc(-c3ccccc3)c(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4c5ccc5c6ccccc6n(-c6ccccc6)c54)c(-c4nc(-c5ccccc5)nc(-c5ccccc5)n4)c3)c2)cc1]

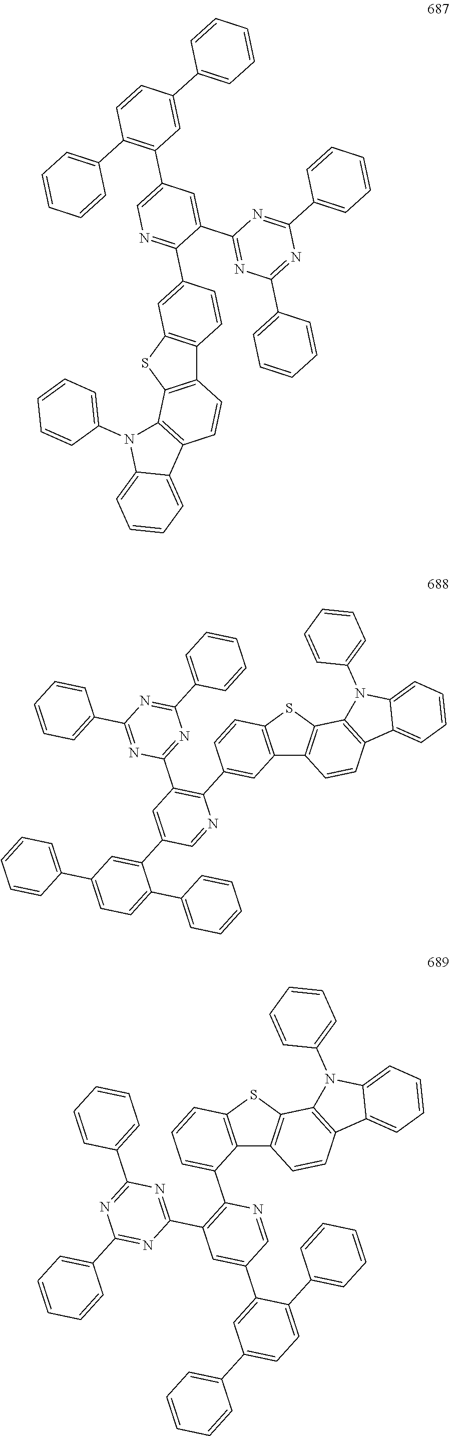 [SMILES: c1ccc(-c2ccc(-c3ccccc3)c(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c8ccccc8n(-c8ccccc8)c76)c45)c(-c4nc(-c5ccccc5)nc(-c5ccccc5)n4)c3)c2)cc1]

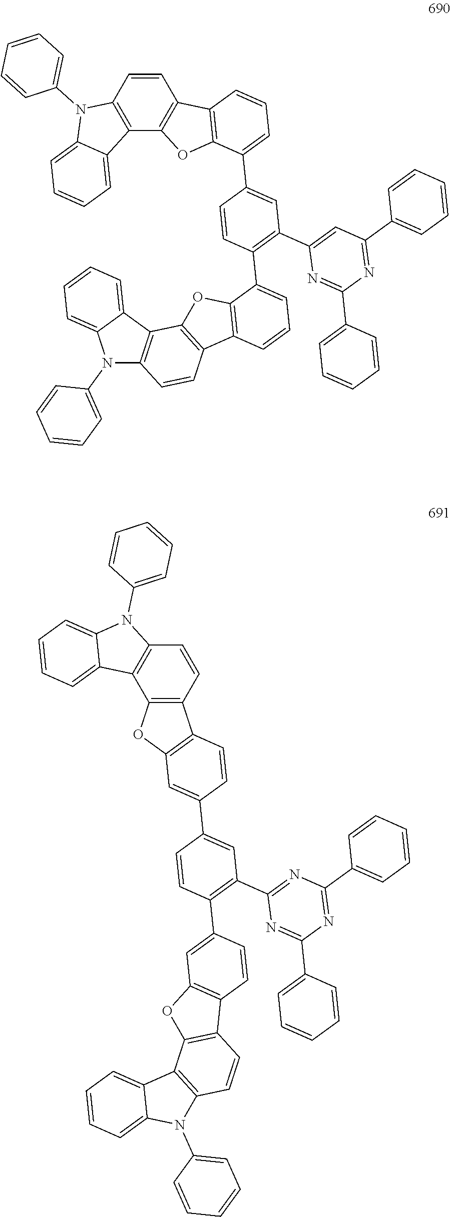 [SMILES: c1ccc(-c2cc(-c3cc(-c4cccc5c4oc4c5ccc5c4c4ccccc4n5-c4ccccc4)ccc3-c3cccc4c3oc3c4ccc4c3c3ccccc3n4-c3ccccc3)nc(-c3ccccc3)n2)cc1 ; c1ccc(-c2nc(-c3ccccc3)nc(-c3cc(-c4ccc5c(c4)oc4c5ccc5c4c4ccccc4n5-c4ccccc4)ccc3-c3ccc4c(c3)oc3c4ccc4c3c3ccccc3n4-c3ccccc3)n2)cc1]

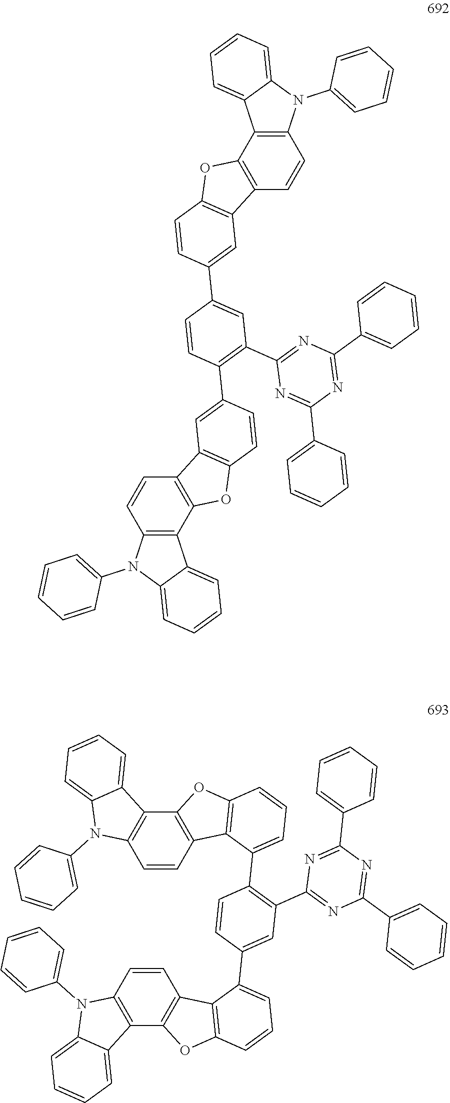 [SMILES: c1ccc(-c2nc(-c3ccccc3)nc(-c3cc(-c4ccc5oc6c(ccc7c6c6ccccc6n7-c6ccccc6)c5c4)ccc3-c3ccc4oc5c(ccc6c5c5ccccc5n6-c5ccccc5)c4c3)n2)cc1 ; c1ccc(-c2nc(-c3ccccc3)nc(-c3cc(-c4cccc5oc6c(ccc7c6c6ccccc6n7-c6ccccc6)c45)ccc3-c3cccc4oc5c(ccc6c5c5ccccc5n6-c5ccccc5)c34)n2)cc1]

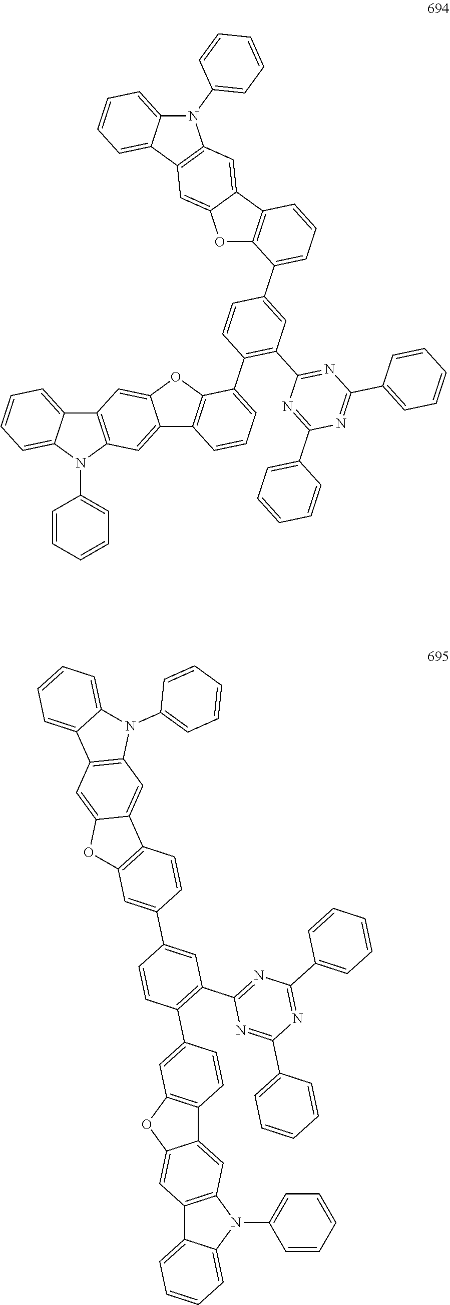 [SMILES: c1ccc(-c2nc(-c3ccccc3)nc(-c3cc(-c4cccc5c4oc4cc6c7ccccc7n(-c7ccccc7)c6cc45)ccc3-c3cccc4c3oc3cc5c6ccccc6n(-c6ccccc6)c5cc34)n2)cc1 ; c1ccc(-c2nc(-c3ccccc3)nc(-c3cc(-c4ccc5c(c4)oc4cc6c7ccccc7n(-c7ccccc7)c6cc45)ccc3-c3ccc4c(c3)oc3cc5c6ccccc6n(-c6ccccc6)c5cc34)n2)cc1]

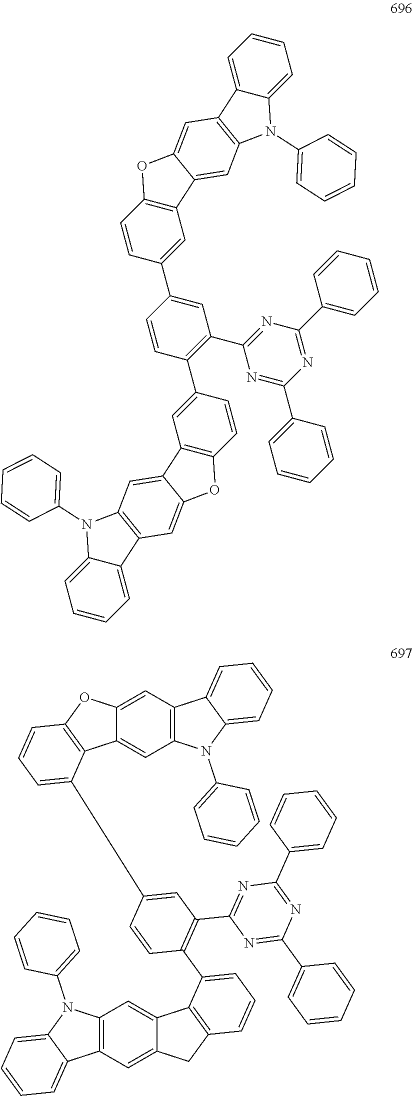 [SMILES: c1ccc(-c2nc(-c3ccccc3)nc(-c3cc(-c4ccc5oc6cc7c8ccccc8n(-c8ccccc8)c7cc6c5c4)ccc3-c3ccc4oc5cc6c7ccccc7n(-c7ccccc7)c6cc5c4c3)n2)cc1 ; c1ccc(-c2nc(-c3ccccc3)nc(-c3cc(-c4cccc5oc6cc7c8ccccc8n(-c8ccccc8)c7cc6c45)ccc3-c3cccc4c3-c3cc5c(cc3C4)c3ccccc3n5-c3ccccc3)n2)cc1]

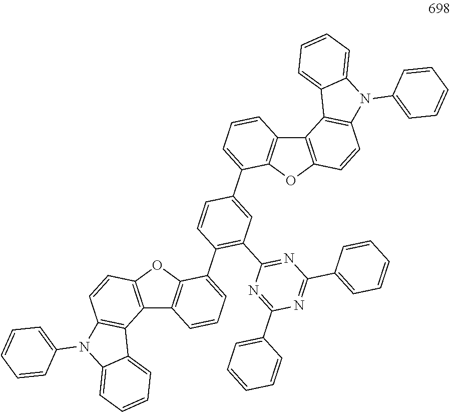 [SMILES: c1ccc(-c2nc(-c3ccccc3)nc(-c3cc(-c4cccc5c4oc4ccc6c(c7ccccc7n6-c6ccccc6)c45)ccc3-c3cccc4c3oc3ccc5c(c6ccccc6n5-c5ccccc5)c34)n2)cc1]

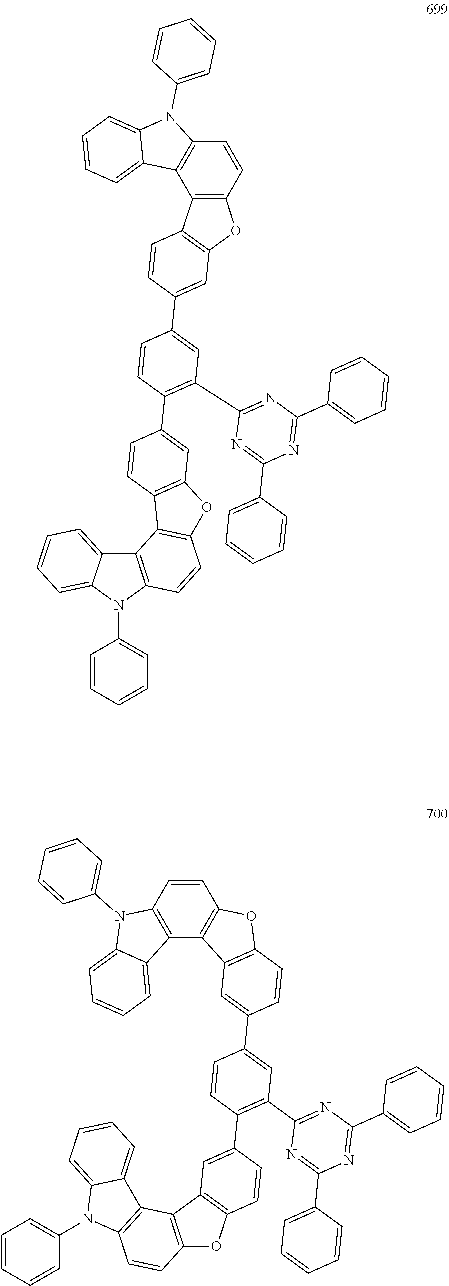 [SMILES: c1ccc(-c2nc(-c3ccccc3)nc(-c3cc(-c4ccc5c(c4)oc4ccc6c(c7ccccc7n6-c6ccccc6)c45)ccc3-c3ccc4c(c3)oc3ccc5c(c6ccccc6n5-c5ccccc5)c34)n2)cc1 ; c1ccc(-c2nc(-c3ccccc3)nc(-c3cc(-c4ccc5oc6ccc7c(c8ccccc8n7-c7ccccc7)c6c5c4)ccc3-c3ccc4oc5ccc6c(c7ccccc7n6-c6ccccc6)c5c4c3)n2)cc1]

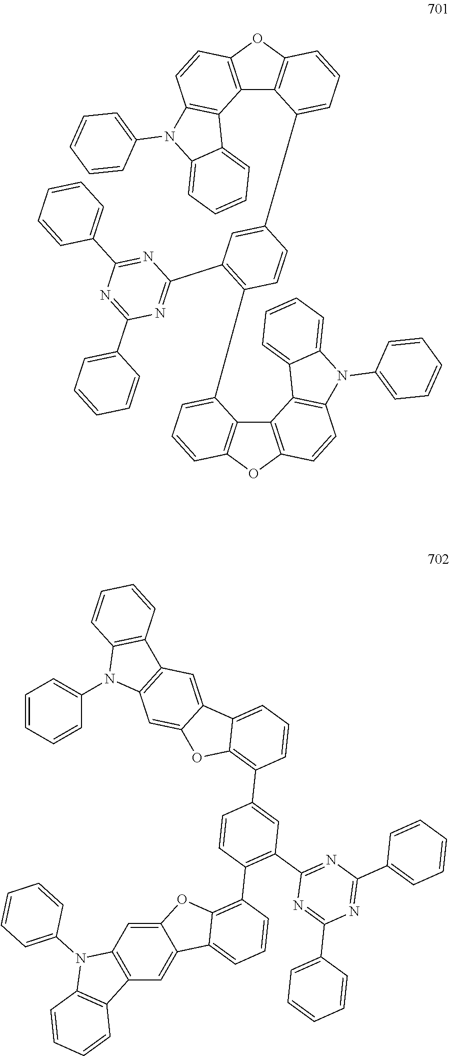 [SMILES: c1ccc(-c2nc(-c3ccccc3)nc(-c3cc(-c4cccc5oc6ccc7c(c8ccccc8n7-c7ccccc7)c6c45)ccc3-c3cccc4oc5ccc6c(c7ccccc7n6-c6ccccc6)c5c34)n2)cc1 ; c1ccc(-c2nc(-c3ccccc3)nc(-c3cc(-c4cccc5c4oc4cc6c(cc45)c4ccccc4n6-c4ccccc4)ccc3-c3cccc4c3oc3cc5c(cc34)c3ccccc3n5-c3ccccc3)n2)cc1]

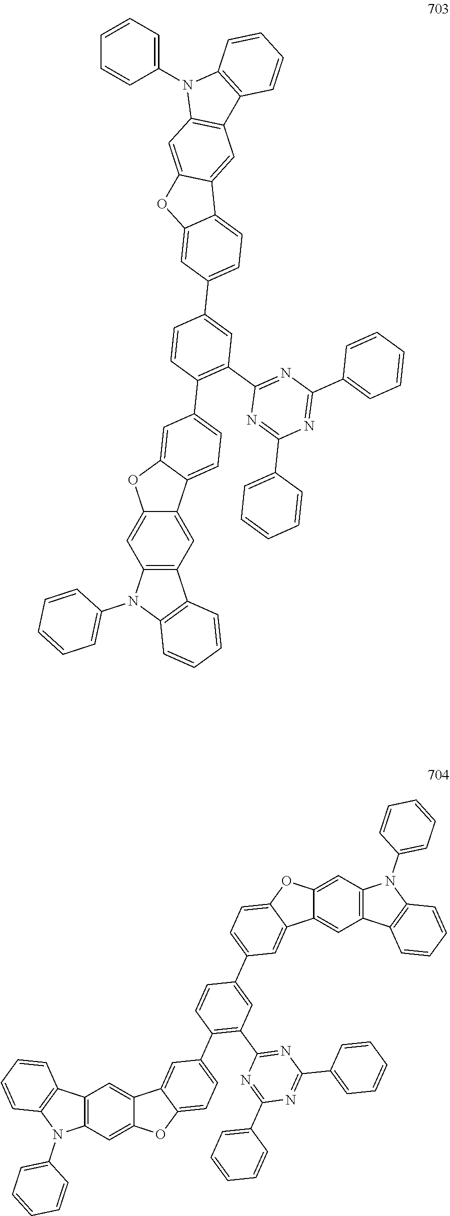 [SMILES: c1ccc(-c2nc(-c3ccccc3)nc(-c3cc(-c4ccc5c(c4)oc4cc6c(cc45)c4ccccc4n6-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4ccc5oc6cc7c(cc6c5c4)c4ccccc4n7-c4ccccc4)ccc3-c3ccc4oc5cc6c(cc5c4c3)c3ccccc3n6-c3ccccc3)n2)cc1]

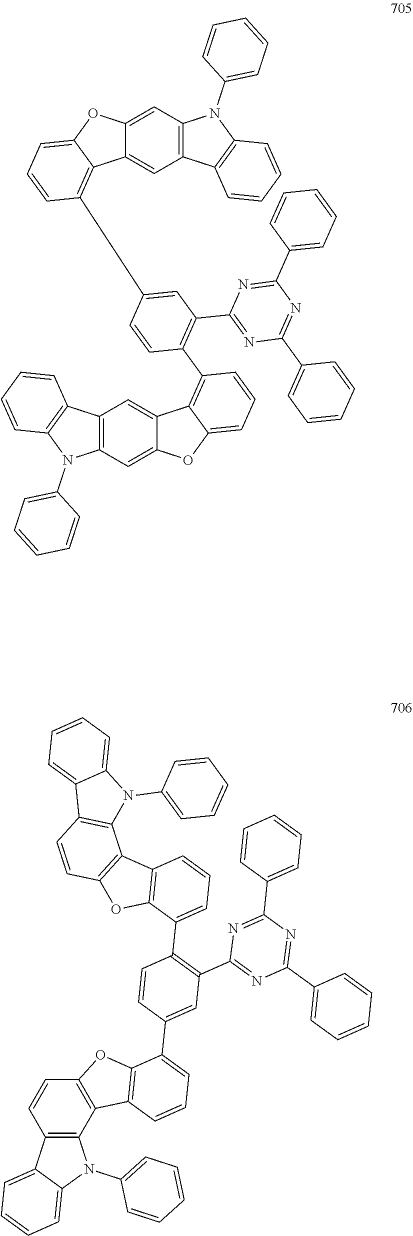 [SMILES: c1ccc(-c2nc(-c3ccccc3)nc(-c3cc(-c4cccc5oc6cc7c(cc6c45)c4ccccc4n7-c4ccccc4)ccc3-c3cccc4oc5cc6c(cc5c34)c3ccccc3n6-c3ccccc3)n2)cc1 ; c1ccc(-c2nc(-c3ccccc3)nc(-c3cc(-c4cccc5c4oc4ccc6c7ccccc7n(-c7ccccc7)c6c45)ccc3-c3cccc4c3oc3ccc5c6ccccc6n(-c6ccccc6)c5c34)n2)cc1]

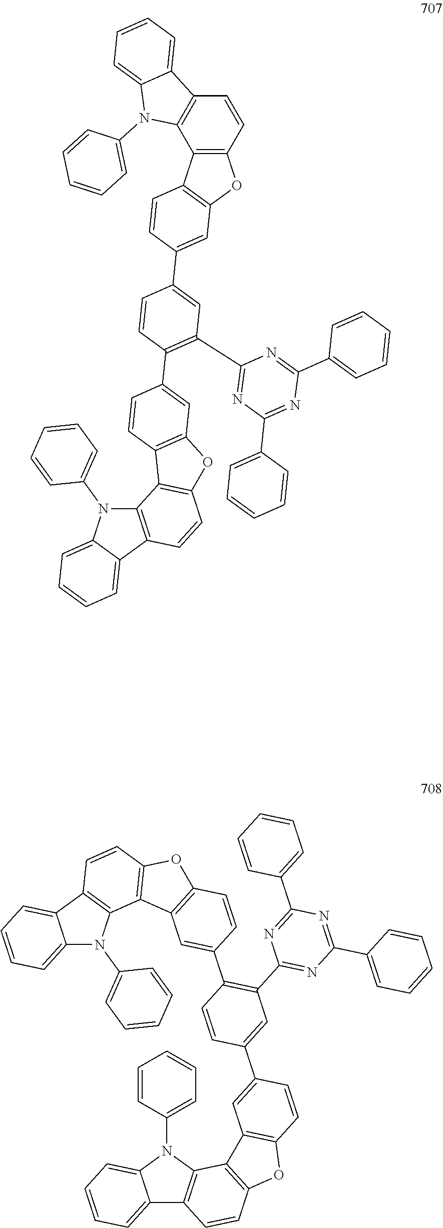 [SMILES: c1ccc(-c2nc(-c3ccccc3)nc(-c3cc(-c4ccc5c(c4)oc4ccc6c7ccccc7n(-c7ccccc7)c6c45)ccc3-c3ccc4c(c3)oc3ccc5c6ccccc6n(-c6ccccc6)c5c34)n2)cc1 ; c1ccc(-c2nc(-c3ccccc3)nc(-c3cc(-c4ccc5oc6ccc7c8ccccc8n(-c8ccccc8)c7c6c5c4)ccc3-c3ccc4oc5ccc6c7ccccc7n(-c7ccccc7)c6c5c4c3)n2)cc1]

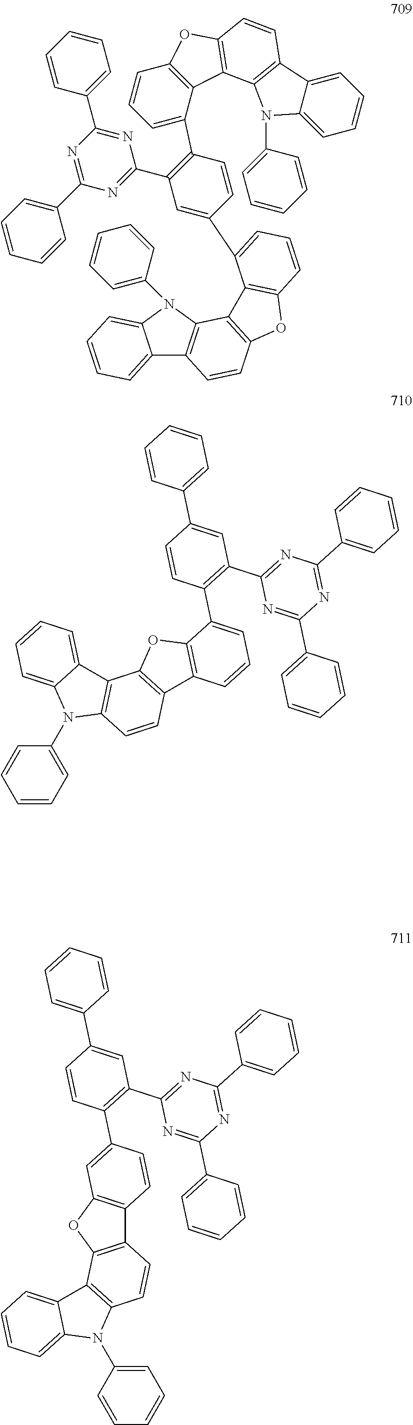 [SMILES: c1ccc(-c2nc(-c3ccccc3)nc(-c3cc(-c4cccc5oc6ccc7c8ccccc8n(-c8ccccc8)c7c6c45)ccc3-c3cccc4oc5ccc6c7ccccc7n(-c7ccccc7)c6c5c34)n2)cc1 ; c1ccc(-c2ccc(-c3cccc4c3oc3c4ccc4c3c3ccccc3n4-c3ccccc3)c(-c3nc(-c4ccccc4)nc(-c4ccccc4)n3)c2)cc1 ; c1ccc(-c2ccc(-c3ccc4c(c3)oc3c4ccc4c3c3ccccc3n4-c3ccccc3)c(-c3nc(-c4ccccc4)nc(-c4ccccc4)n3)c2)cc1]

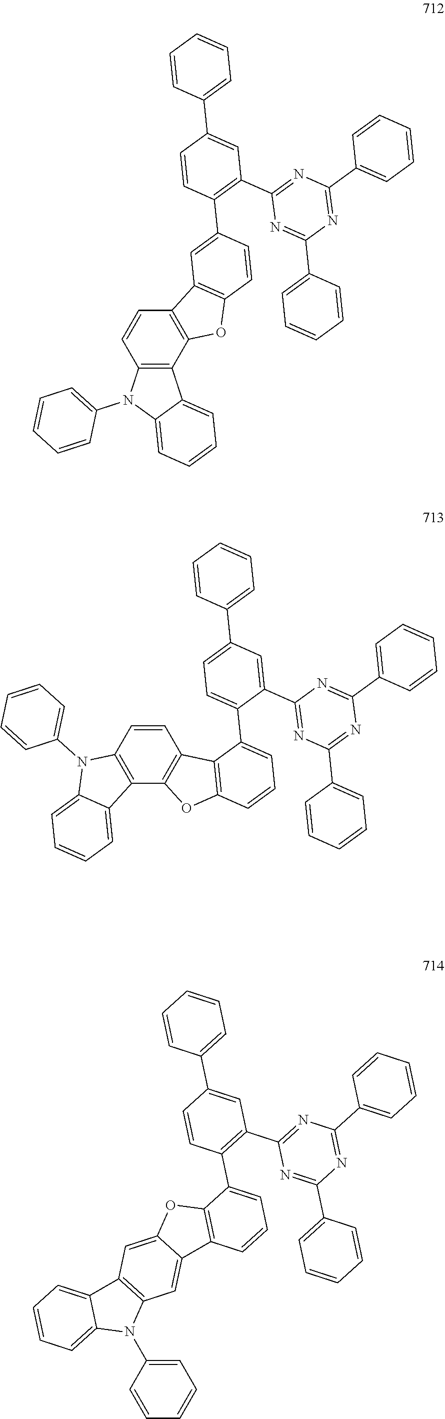 [SMILES: c1ccc(-c2ccc(-c3ccc4oc5c(ccc6c5c5ccccc5n6-c5ccccc5)c4c3)c(-c3nc(-c4ccccc4)nc(-c4ccccc4)n3)c2)cc1 ; c1ccc(-c2ccc(-c3cccc4oc5c(ccc6c5c5ccccc5n6-c5ccccc5)c34)c(-c3nc(-c4ccccc4)nc(-c4ccccc4)n3)c2)cc1 ; c1ccc(-c2ccc(-c3cccc4c3oc3cc5c6ccccc6n(-c6ccccc6)c5cc34)c(-c3nc(-c4ccccc4)nc(-c4ccccc4)n3)c2)cc1]

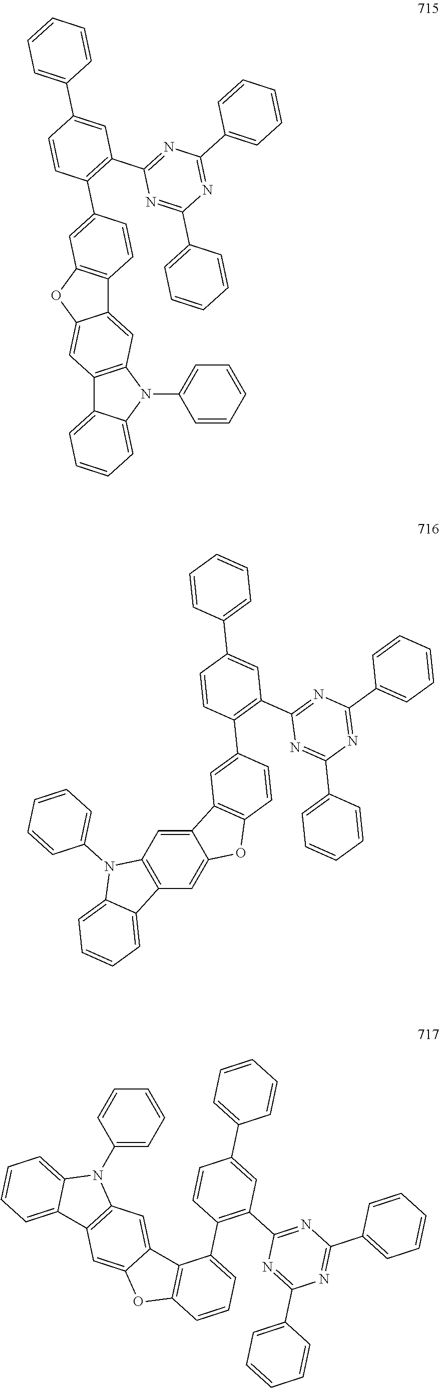 [SMILES: c1ccc(-c2ccc(-c3ccc4c(c3)oc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4oc5cc6c7ccccc7n(-c7ccccc7)c6cc5c4c3)c(-c3nc(-c4ccccc4)nc(-c4ccccc4)n3)c2)cc1 ; c1ccc(-c2ccc(-c3cccc4oc5cc6c7ccccc7n(-c7ccccc7)c6cc5c34)c(-c3nc(-c4ccccc4)nc(-c4ccccc4)n3)c2)cc1]

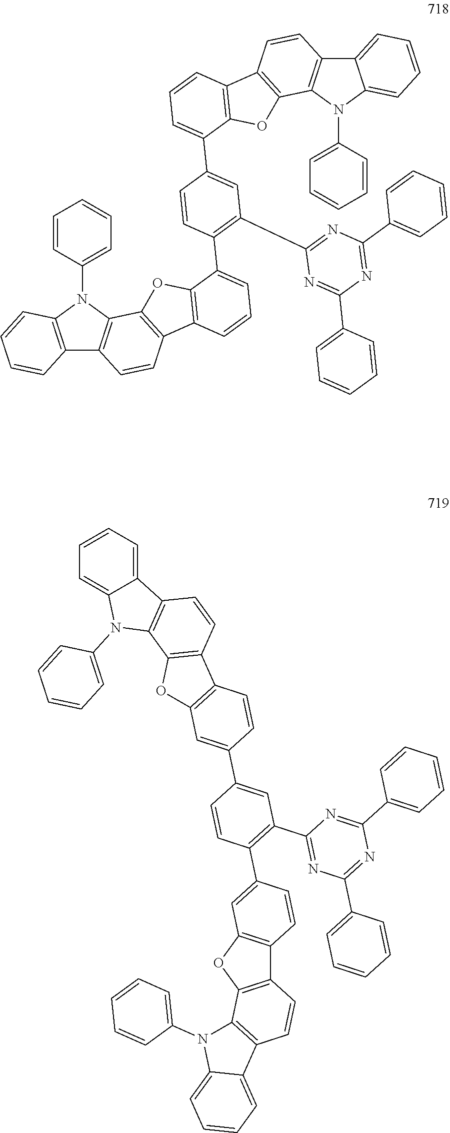 [SMILES: c1ccc(-c2nc(-c3ccccc3)nc(-c3cc(-c4cccc5c4oc4c5ccc5c6ccccc6n(-c6ccccc6)c54)ccc3-c3cccc4c3oc3c4ccc4c5ccccc5n(-c5ccccc5)c43)n2)cc1 ; c1ccc(-c2nc(-c3ccccc3)nc(-c3cc(-c4ccc5c(c4)oc4c5ccc5c6ccccc6n(-c6ccccc6)c54)ccc3-c3ccc4c(c3)oc3c4ccc4c5ccccc5n(-c5ccccc5)c43)n2)cc1]

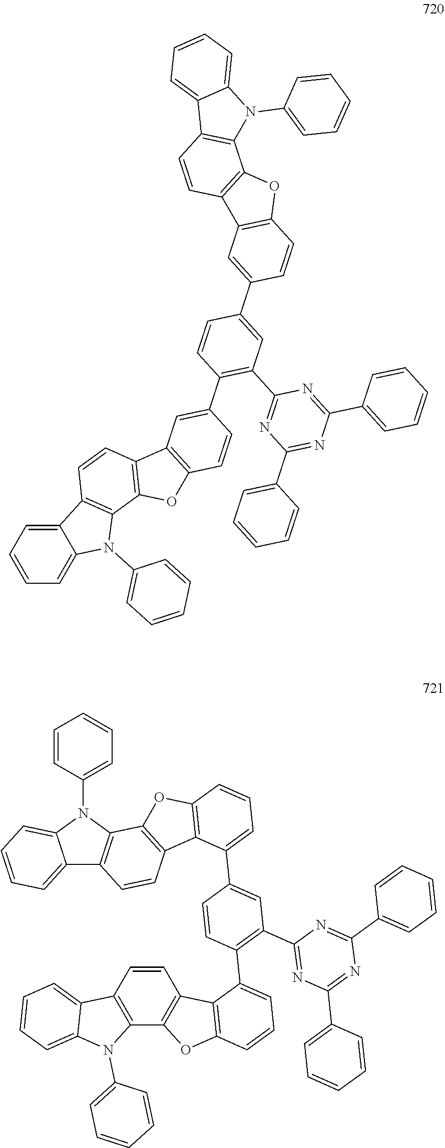 [SMILES: c1ccc(-c2nc(-c3ccccc3)nc(-c3cc(-c4ccc5oc6c(ccc7c8ccccc8n(-c8ccccc8)c76)c5c4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4cccc5oc6c(ccc7c8ccccc8n(-c8ccccc8)c76)c45)ccc3-c3cccc4oc5c(ccc6c7ccccc7n(-c7ccccc7)c65)c34)n2)cc1]

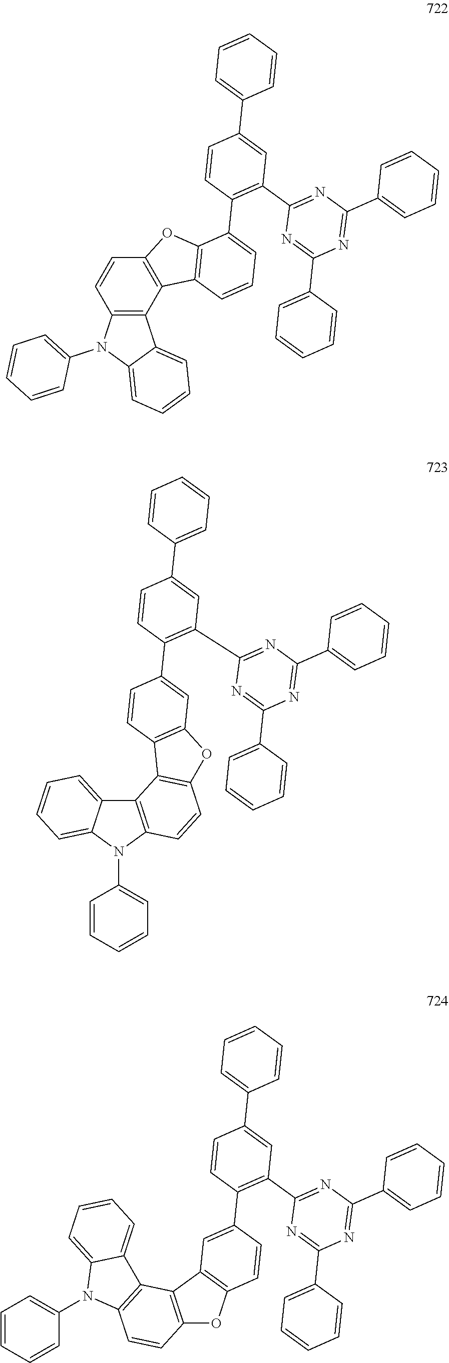 [SMILES: c1ccc(-c2ccc(-c3cccc4c3oc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4c(c3)oc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4oc5ccc6c(c7ccccc7n6-c6ccccc6)c5c4c3)c(-c3nc(-c4ccccc4)nc(-c4ccccc4)n3)c2)cc1]

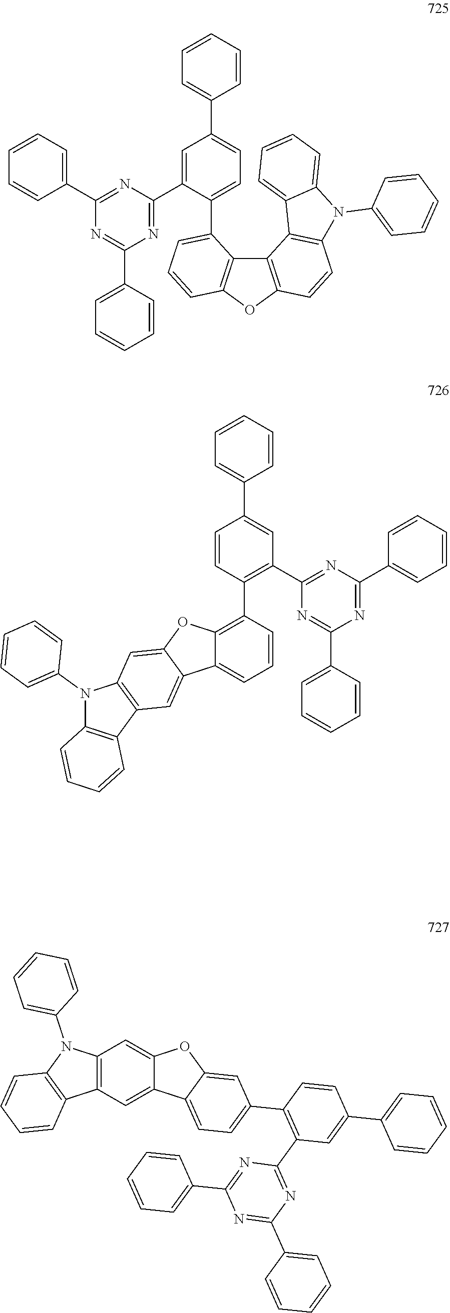 [SMILES: c1ccc(-c2ccc(-c3cccc4oc5ccc6c(c7ccccc7n6-c6ccccc6)c5c34)c(-c3nc(-c4ccccc4)nc(-c4ccccc4)n3)c2)cc1 ; c1ccc(-c2ccc(-c3cccc4c3oc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)oc3cc5c(cc34)c3ccccc3n5-c3ccccc3)c(-c3nc(-c4ccccc4)nc(-c4ccccc4)n3)c2)cc1]

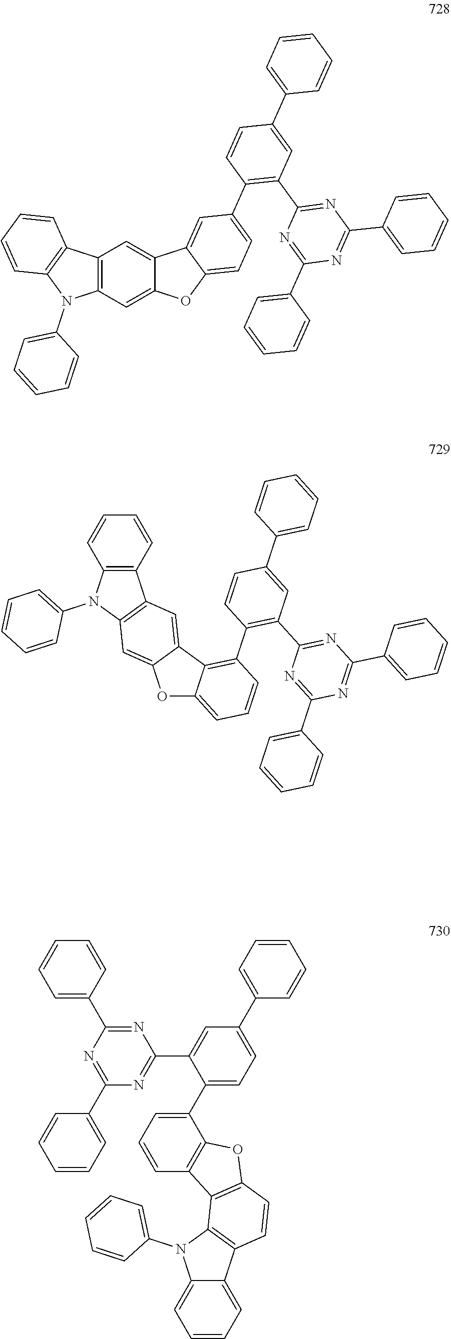 [SMILES: c1ccc(-c2ccc(-c3ccc4oc5cc6c(cc5c4c3)c3ccccc3n6-c3ccccc3)c(-c3nc(-c4ccccc4)nc(-c4ccccc4)n3)c2)cc1 ; c1ccc(-c2ccc(-c3cccc4oc5cc6c(cc5c34)c3ccccc3n6-c3ccccc3)c(-c3nc(-c4ccccc4)nc(-c4ccccc4)n3)c2)cc1 ; c1ccc(-c2ccc(-c3cccc4c3oc3ccc5c6ccccc6n(-c6ccccc6)c5c34)c(-c3nc(-c4ccccc4)nc(-c4ccccc4)n3)c2)cc1]

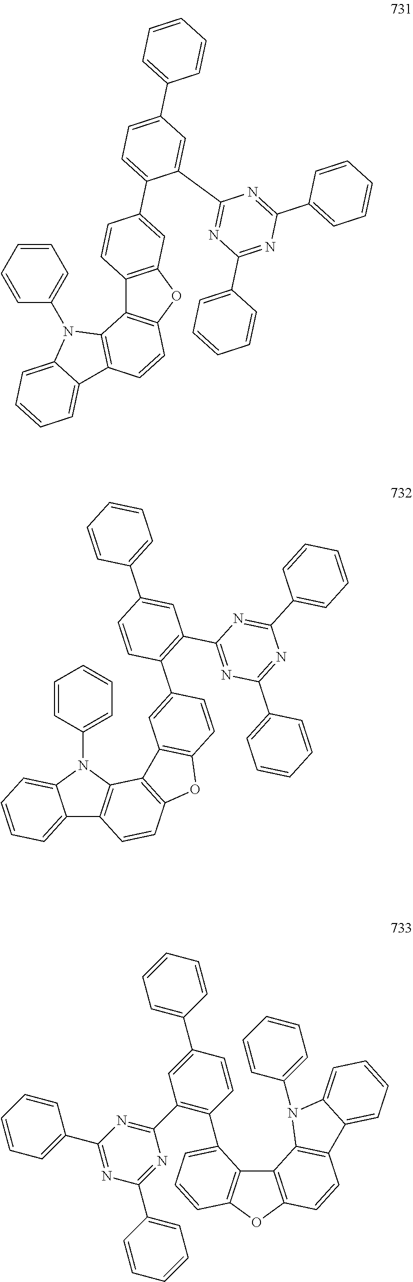 [SMILES: c1ccc(-c2ccc(-c3ccc4c(c3)oc3ccc5c6ccccc6n(-c6ccccc6)c5c34)c(-c3nc(-c4ccccc4)nc(-c4ccccc4)n3)c2)cc1 ; c1ccc(-c2ccc(-c3ccc4oc5ccc6c7ccccc7n(-c7ccccc7)c6c5c4c3)c(-c3nc(-c4ccccc4)nc(-c4ccccc4)n3)c2)cc1 ; c1ccc(-c2ccc(-c3cccc4oc5ccc6c7ccccc7n(-c7ccccc7)c6c5c34)c(-c3nc(-c4ccccc4)nc(-c4ccccc4)n3)c2)cc1]

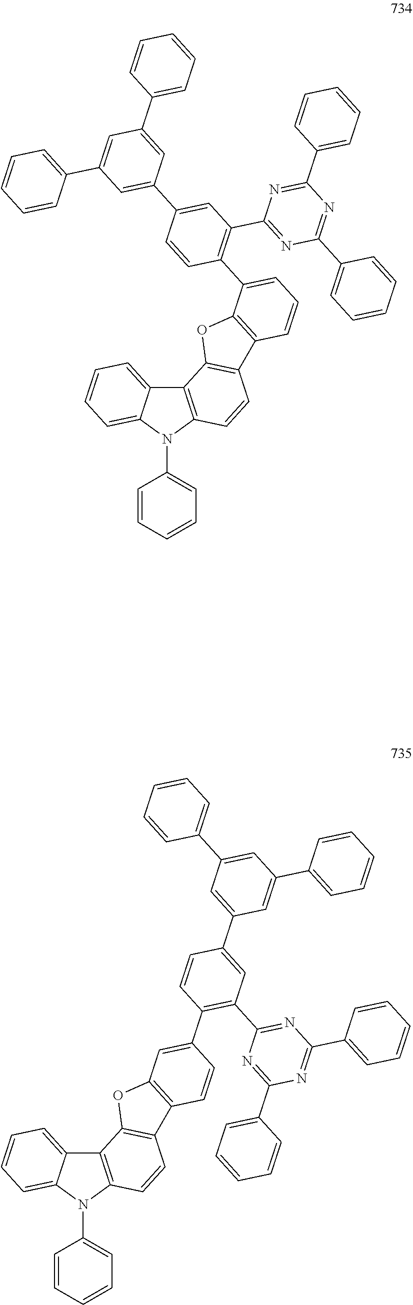 [SMILES: c1ccc(-c2cc(-c3ccccc3)cc(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

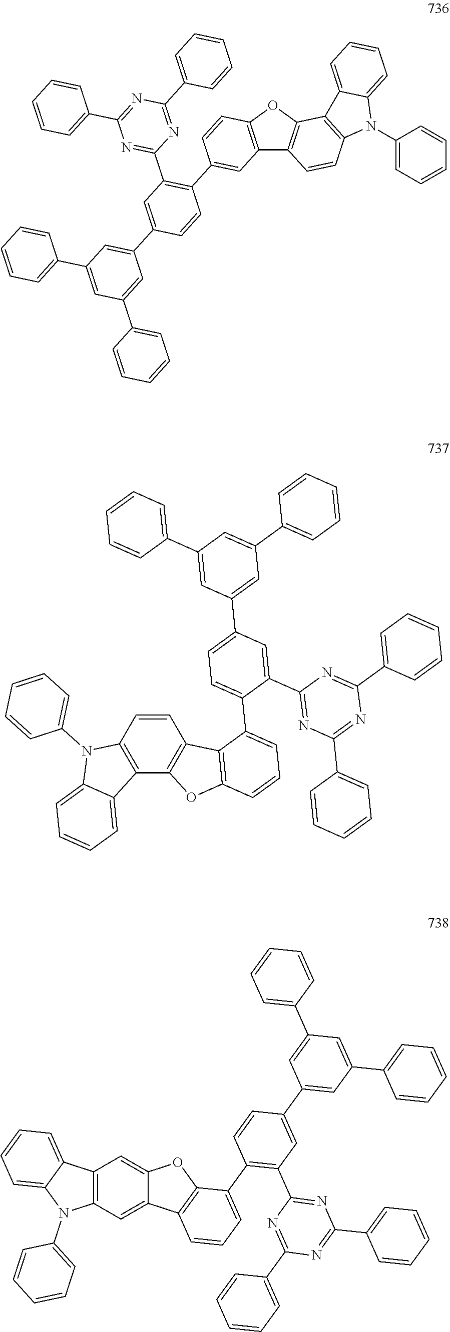 [SMILES: c1ccc(-c2cc(-c3ccccc3)cc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4cccc5c4oc4cc6c7ccccc7n(-c7ccccc7)c6cc45)c(-c4nc(-c5ccccc5)nc(-c5ccccc5)n4)c3)c2)cc1]

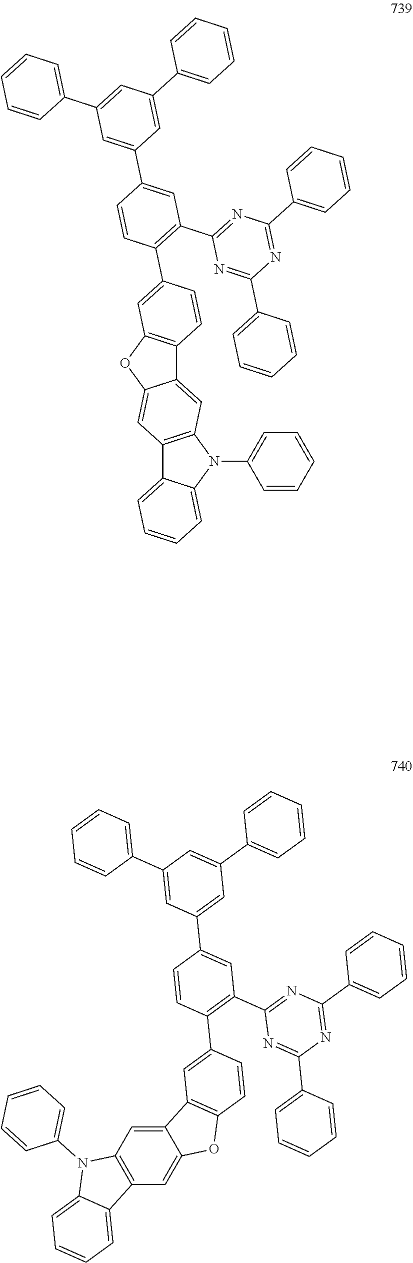 [SMILES: c1ccc(-c2cc(-c3ccccc3)cc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1]

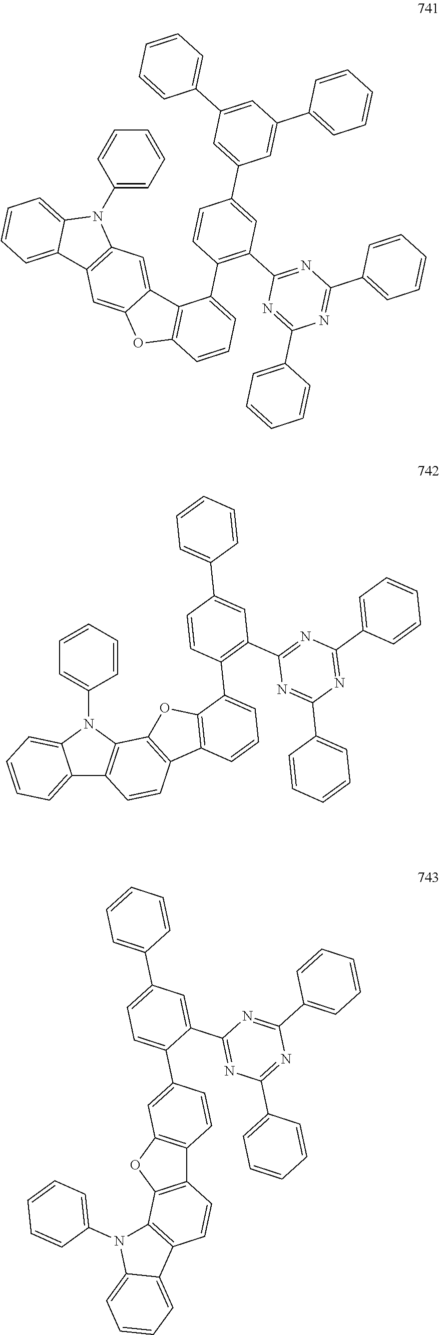 [SMILES: c1ccc(-c2cc(-c3ccccc3)cc(-c3ccc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2ccc(-c3cccc4c3oc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4c(c3)oc3c4ccc4c5ccccc5n(-c5ccccc5)c43)c(-c3nc(-c4ccccc4)nc(-c4ccccc4)n3)c2)cc1]

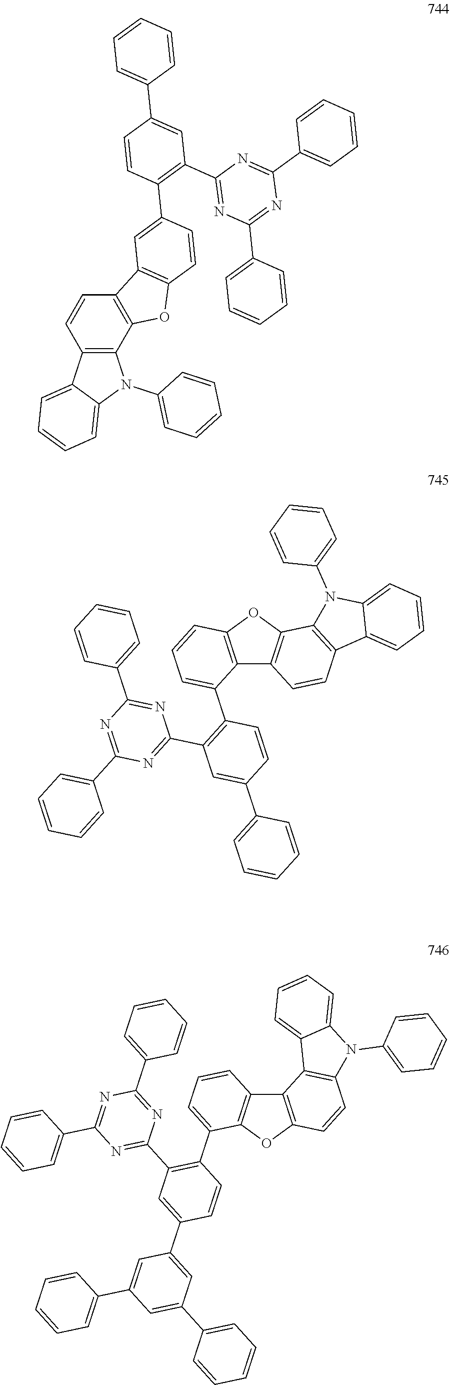 [SMILES: c1ccc(-c2ccc(-c3ccc4oc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4oc5c(ccc6c7ccccc7n(-c7ccccc7)c65)c34)c(-c3nc(-c4ccccc4)nc(-c4ccccc4)n3)c2)cc1 ; c1ccc(-c2cc(-c3ccccc3)cc(-c3ccc(-c4cccc5c4oc4ccc6c(c7ccccc7n6-c6ccccc6)c45)c(-c4nc(-c5ccccc5)nc(-c5ccccc5)n4)c3)c2)cc1]

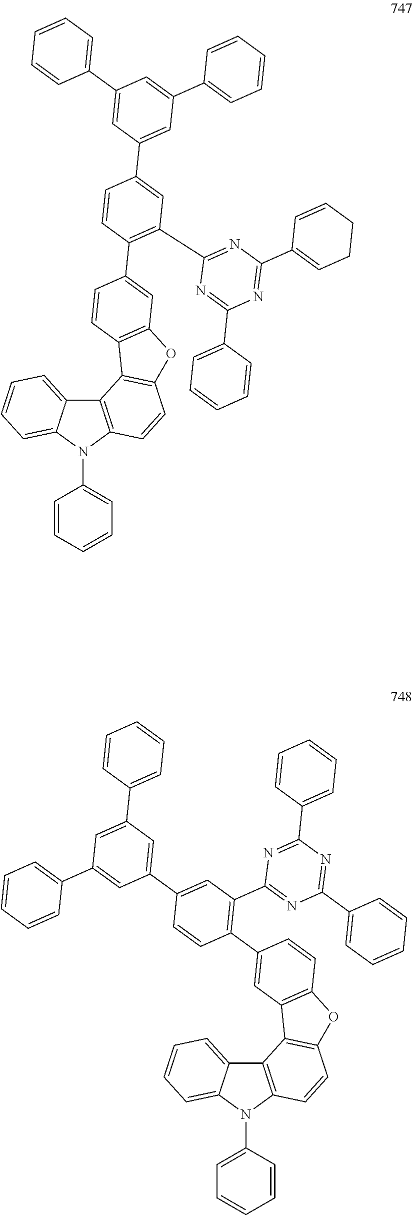 [SMILES: C1=CC(c2nc(-c3ccccc3)nc(-c3cc(-c4cc(-c5ccccc5)cc(-c5ccccc5)c4)ccc3-c3ccc4c(c3)oc3ccc5c(c6ccccc6n5-c5ccccc5)c34)n2)=CCC1 ; c1ccc(-c2cc(-c3ccccc3)cc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1]

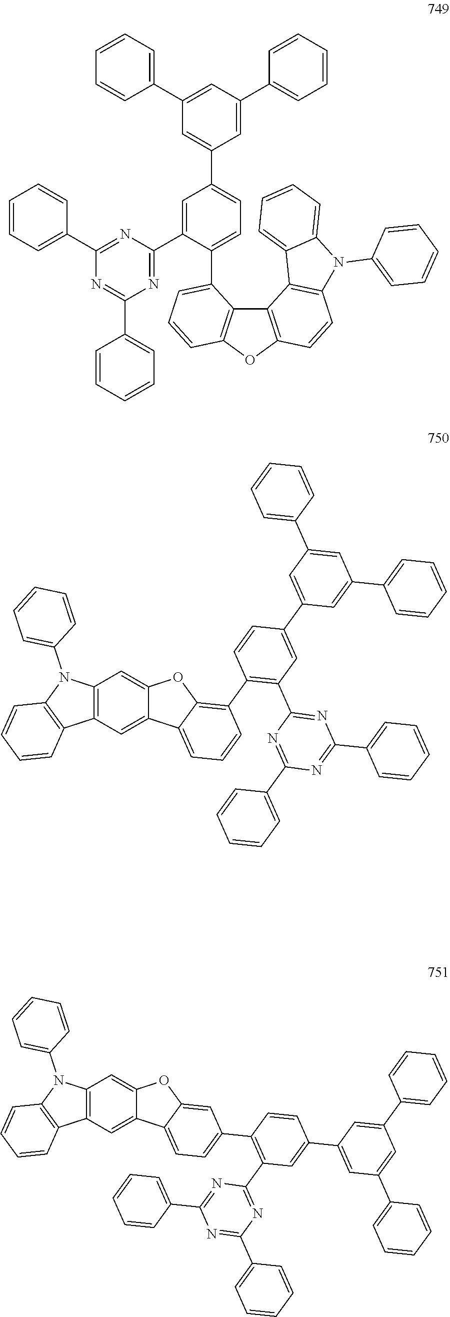 [SMILES: c1ccc(-c2cc(-c3ccccc3)cc(-c3ccc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1]

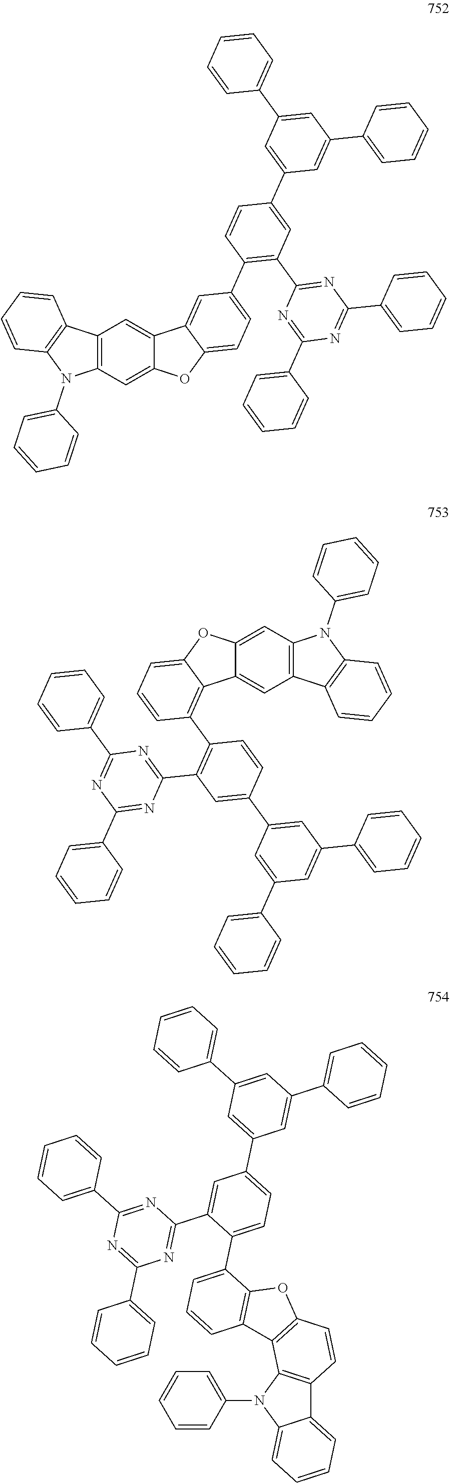 [SMILES: c1ccc(-c2cc(-c3ccccc3)cc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5oc6cc7c(cc6c45)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5c4oc4ccc6c7ccccc7n(-c7ccccc7)c6c45)c(-c4nc(-c5ccccc5)nc(-c5ccccc5)n4)c3)c2)cc1]

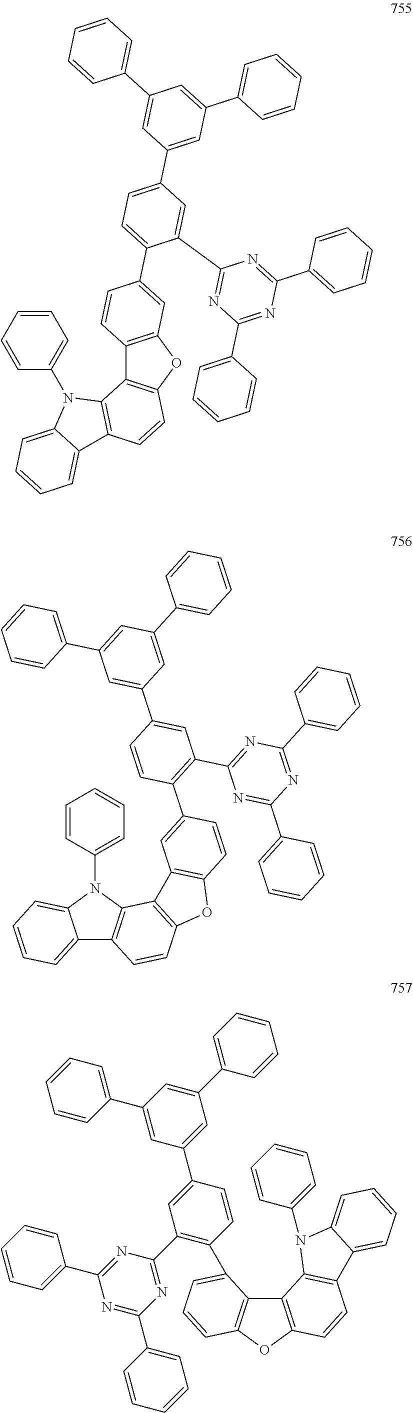 [SMILES: c1ccc(-c2cc(-c3ccccc3)cc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6ccc7c8ccccc8n(-c8ccccc8)c7c6c45)c(-c4nc(-c5ccccc5)nc(-c5ccccc5)n4)c3)c2)cc1]

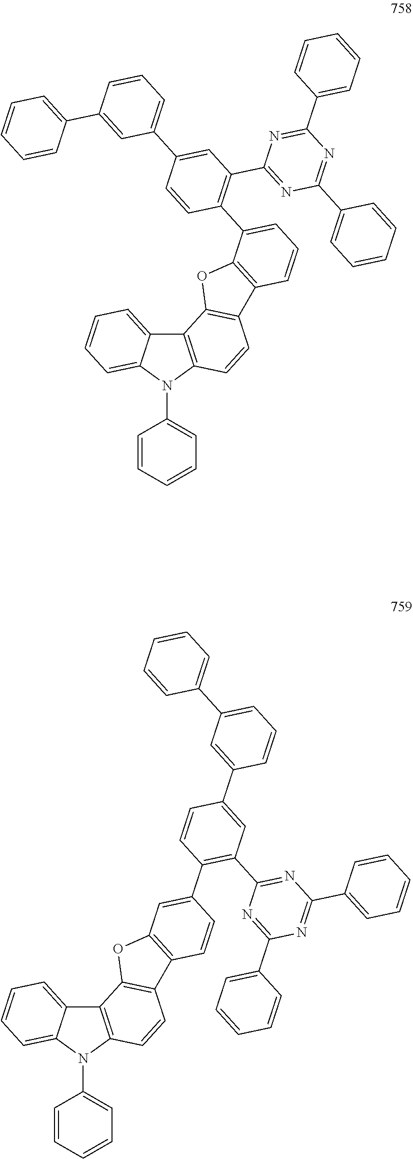 [SMILES: c1ccc(-c2cccc(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cccc(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

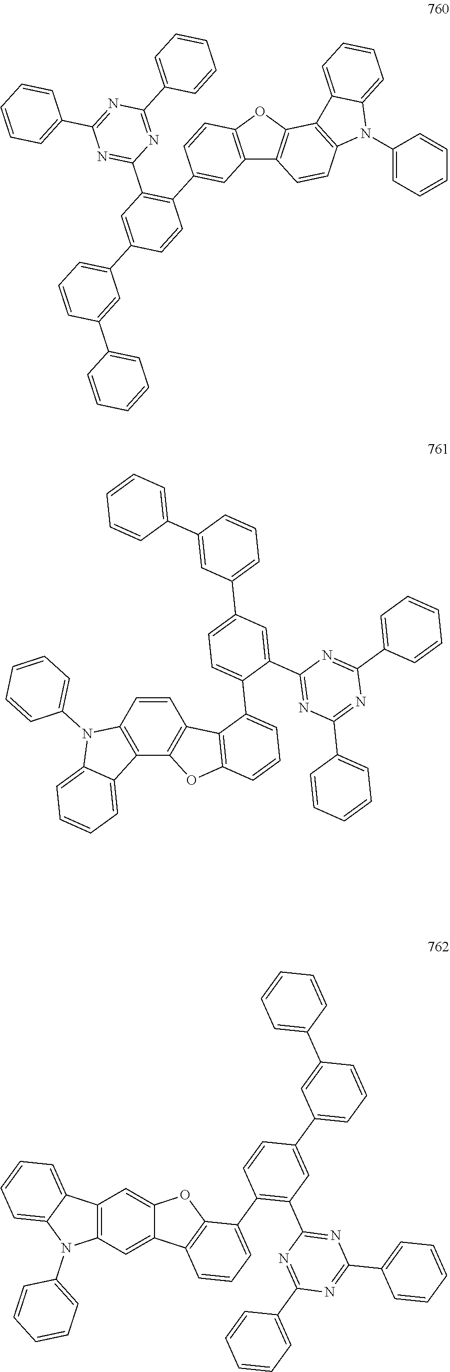 [SMILES: c1ccc(-c2cccc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3ccc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2cccc(-c3ccc(-c4cccc5c4oc4cc6c7ccccc7n(-c7ccccc7)c6cc45)c(-c4nc(-c5ccccc5)nc(-c5ccccc5)n4)c3)c2)cc1]

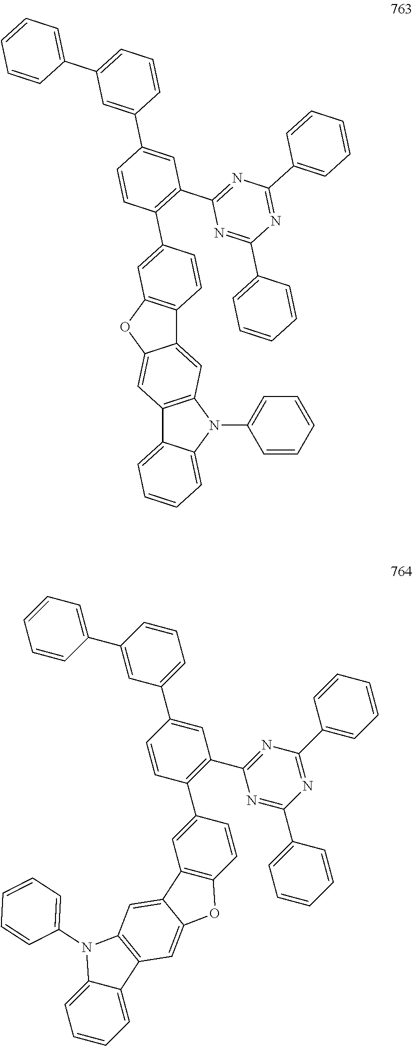 [SMILES: c1ccc(-c2cccc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1]

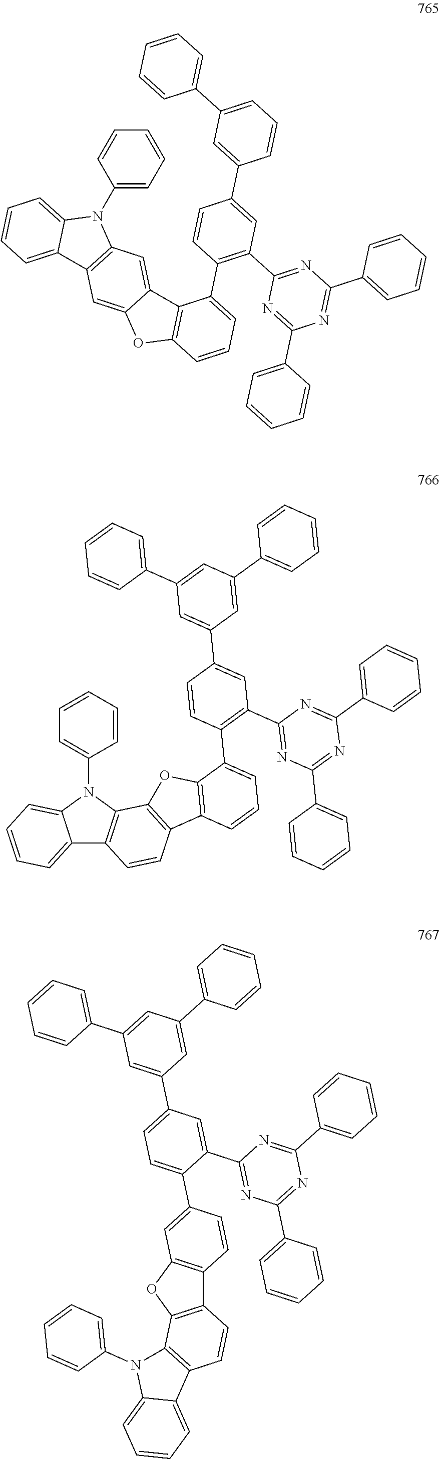 [SMILES: c1ccc(-c2cccc(-c3ccc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2cc(-c3ccccc3)cc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1]

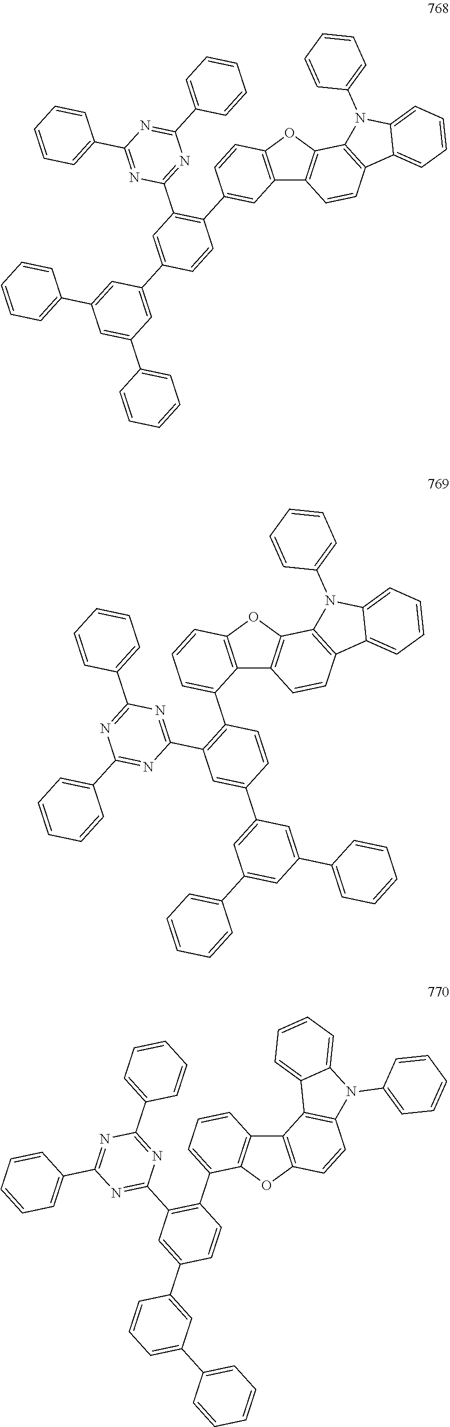 [SMILES: c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3ccc(-c4cccc5c4oc4ccc6c(c7ccccc7n6-c6ccccc6)c45)c(-c4nc(-c5ccccc5)nc(-c5ccccc5)n4)c3)c2)cc1]

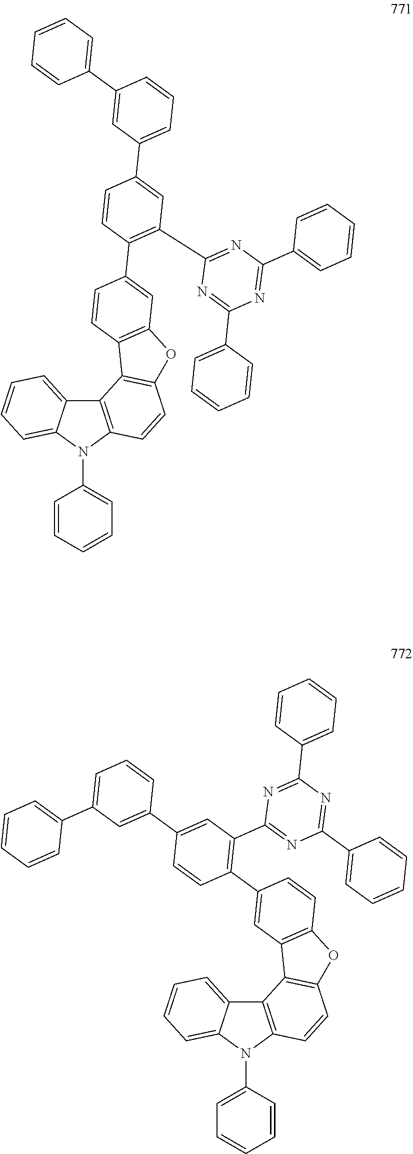 [SMILES: c1ccc(-c2cccc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1]

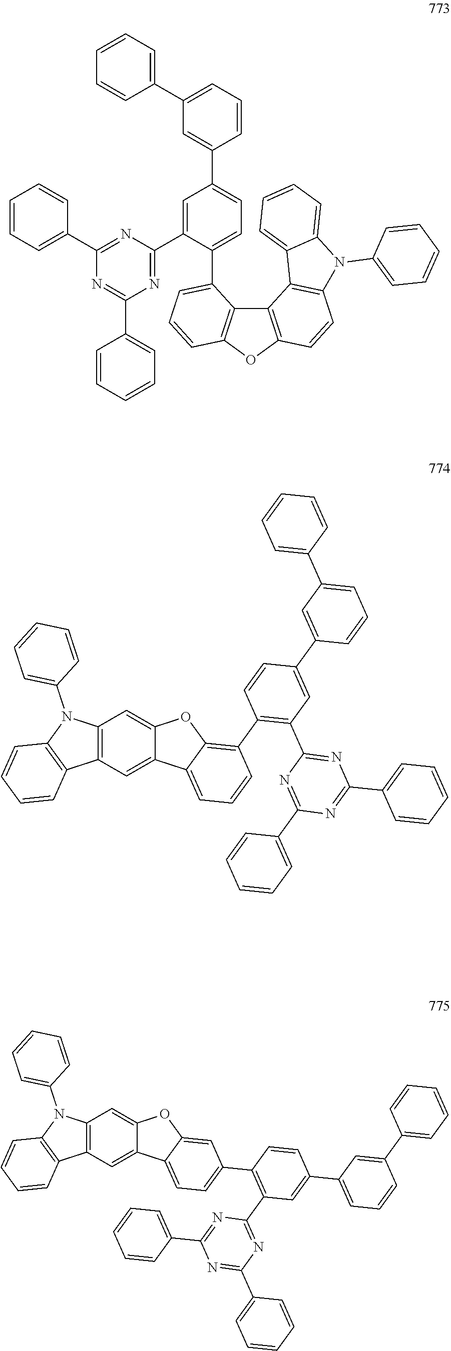 [SMILES: c1ccc(-c2cccc(-c3ccc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1]

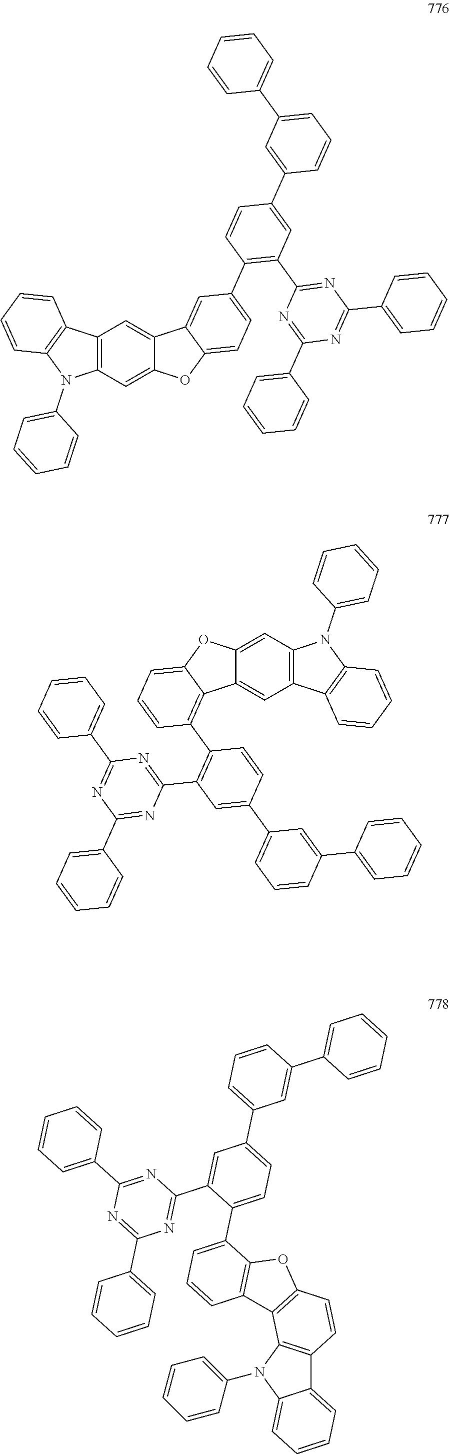 [SMILES: c1ccc(-c2cccc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5oc6cc7c(cc6c45)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5c4oc4ccc6c7ccccc7n(-c7ccccc7)c6c45)c(-c4nc(-c5ccccc5)nc(-c5ccccc5)n4)c3)c2)cc1]

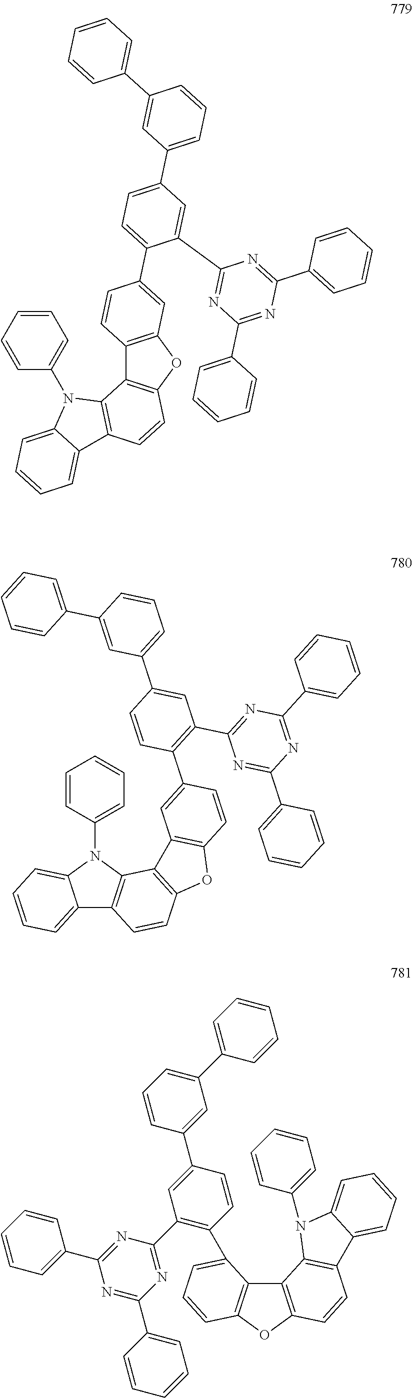 [SMILES: c1ccc(-c2cccc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3ccc(-c4cccc5oc6ccc7c8ccccc8n(-c8ccccc8)c7c6c45)c(-c4nc(-c5ccccc5)nc(-c5ccccc5)n4)c3)c2)cc1]

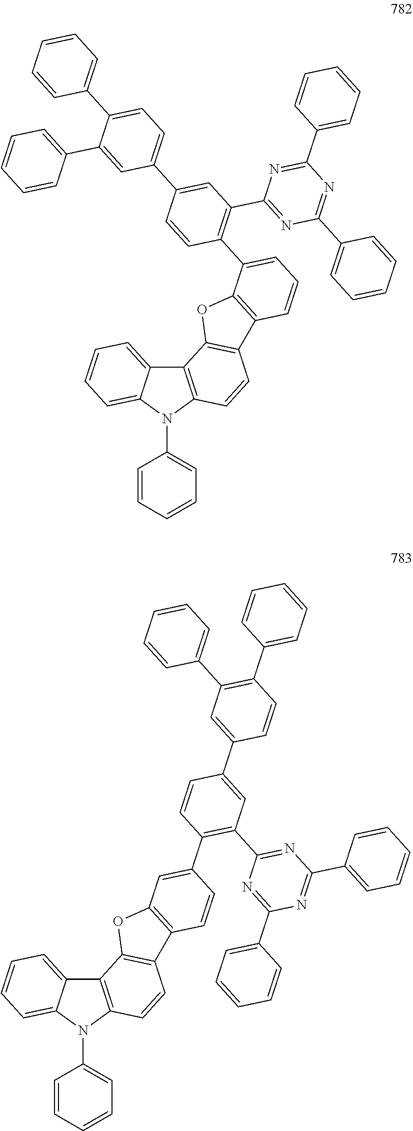 [SMILES: c1ccc(-c2nc(-c3ccccc3)nc(-c3cc(-c4ccc(-c5ccccc5)c(-c5ccccc5)c4)ccc3-c3cccc4c3oc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3c4ccc4c3c3ccccc3n4-c3ccccc3)n2)cc1]

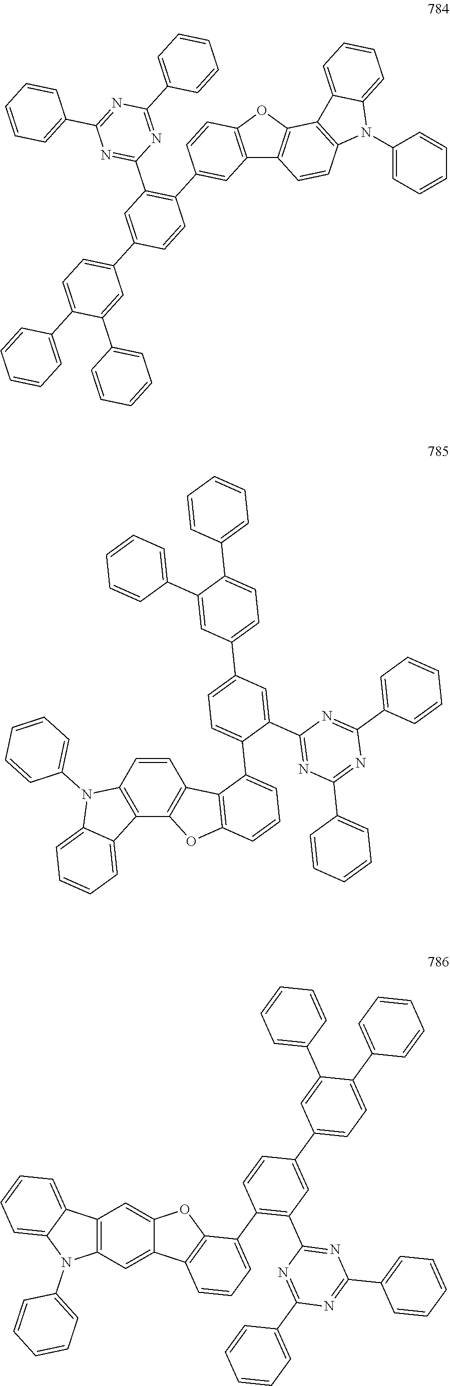 [SMILES: c1ccc(-c2nc(-c3ccccc3)nc(-c3cc(-c4ccc(-c5ccccc5)c(-c5ccccc5)c4)ccc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4oc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)ccc3-c3cccc4c3oc3cc5c6ccccc6n(-c6ccccc6)c5cc34)n2)cc1]

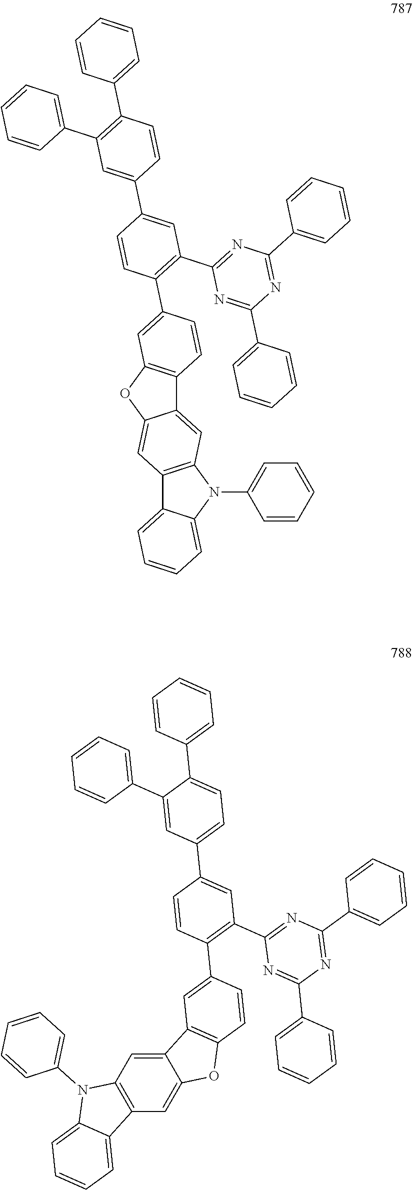 [SMILES: c1ccc(-c2nc(-c3ccccc3)nc(-c3cc(-c4ccc(-c5ccccc5)c(-c5ccccc5)c4)ccc3-c3ccc4c(c3)oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)ccc3-c3ccc4oc5cc6c7ccccc7n(-c7ccccc7)c6cc5c4c3)n2)cc1]

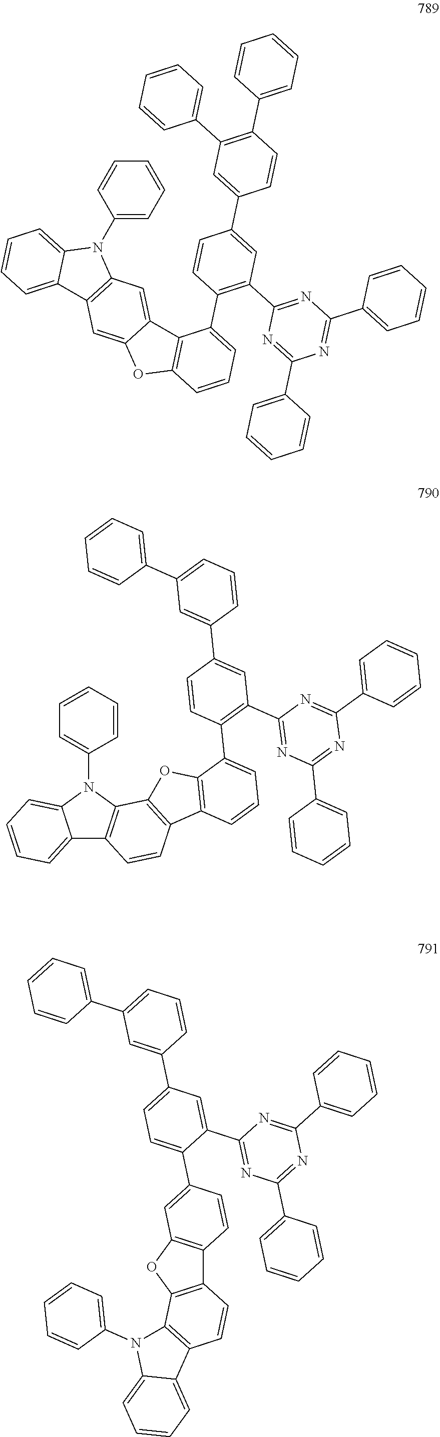 [SMILES: c1ccc(-c2nc(-c3ccccc3)nc(-c3cc(-c4ccc(-c5ccccc5)c(-c5ccccc5)c4)ccc3-c3cccc4oc5cc6c7ccccc7n(-c7ccccc7)c6cc5c34)n2)cc1 ; c1ccc(-c2cccc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1]

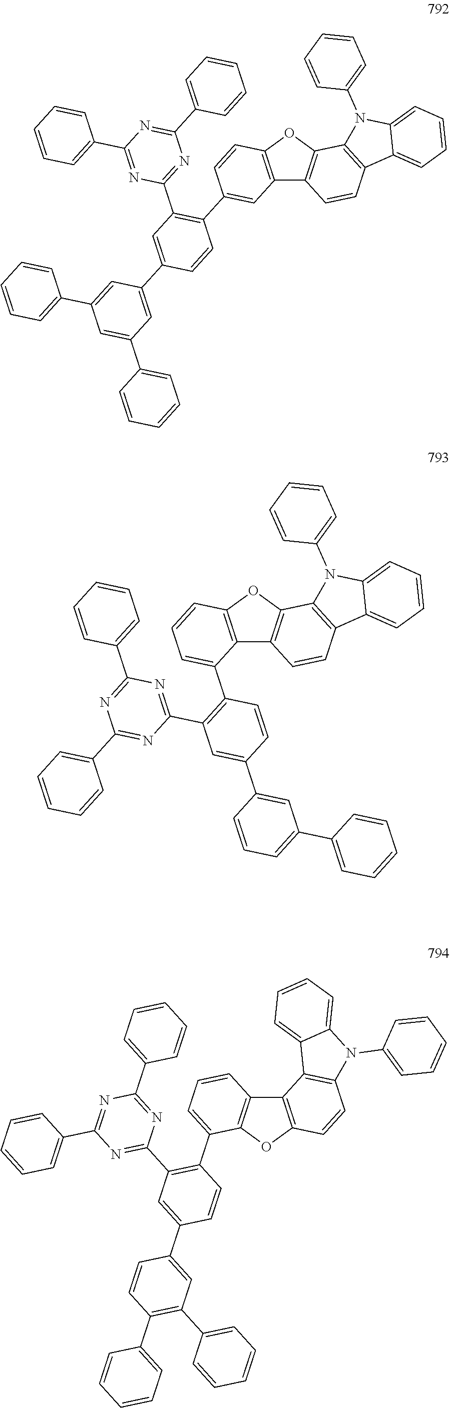 [SMILES: c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3oc3ccc5c(c6ccccc6n5-c5ccccc5)c34)n2)cc1]

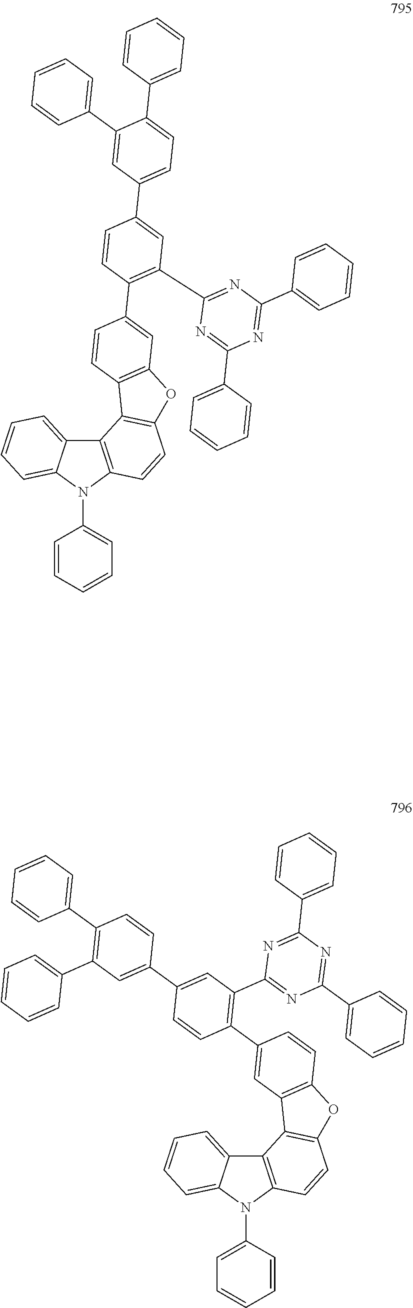 [SMILES: c1ccc(-c2nc(-c3ccccc3)nc(-c3cc(-c4ccc(-c5ccccc5)c(-c5ccccc5)c4)ccc3-c3ccc4c(c3)oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)ccc3-c3ccc4oc5ccc6c(c7ccccc7n6-c6ccccc6)c5c4c3)n2)cc1]

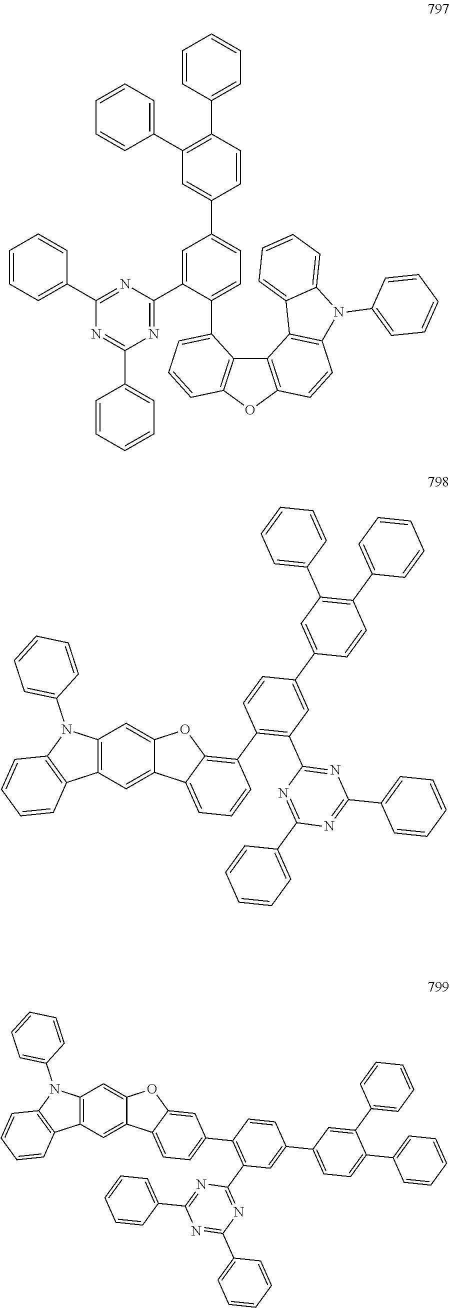 [SMILES: c1ccc(-c2nc(-c3ccccc3)nc(-c3cc(-c4ccc(-c5ccccc5)c(-c5ccccc5)c4)ccc3-c3cccc4oc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3cccc4c3oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3cc5c(cc34)c3ccccc3n5-c3ccccc3)n2)cc1]

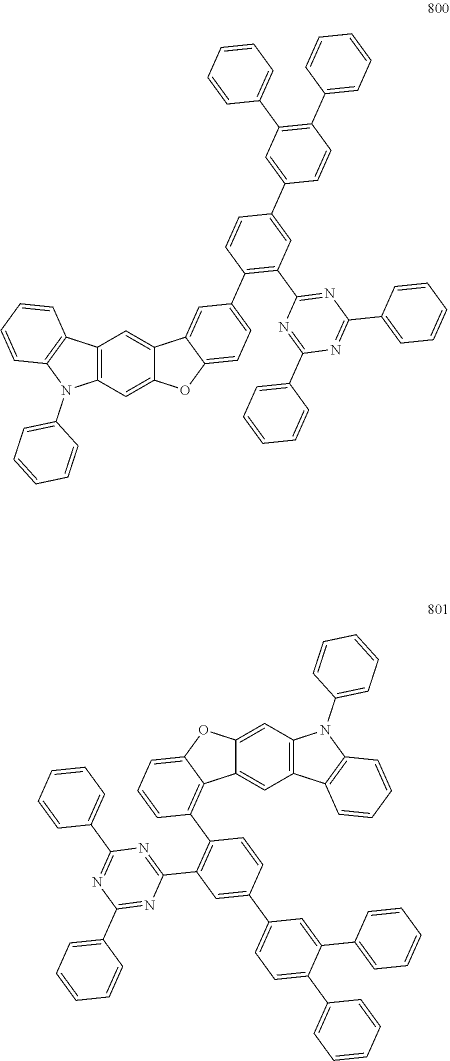 [SMILES: c1ccc(-c2nc(-c3ccccc3)nc(-c3cc(-c4ccc(-c5ccccc5)c(-c5ccccc5)c4)ccc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4oc5cc6c(cc5c34)c3ccccc3n6-c3ccccc3)n2)cc1]

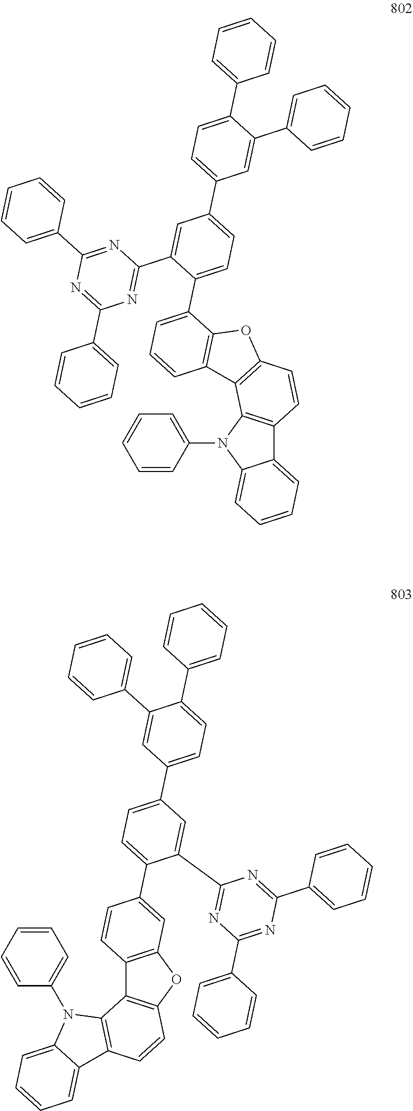 [SMILES: c1ccc(-c2nc(-c3ccccc3)nc(-c3cc(-c4ccc(-c5ccccc5)c(-c5ccccc5)c4)ccc3-c3cccc4c3oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3ccc4c(c3)oc3ccc5c6ccccc6n(-c6ccccc6)c5c34)n2)cc1]

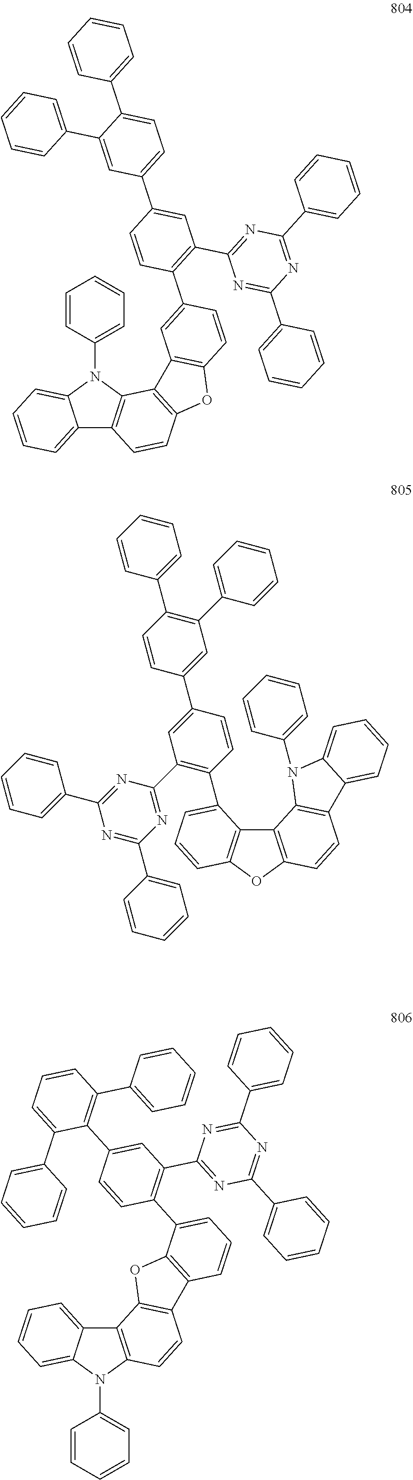 [SMILES: c1ccc(-c2nc(-c3ccccc3)nc(-c3cc(-c4ccc(-c5ccccc5)c(-c5ccccc5)c4)ccc3-c3ccc4oc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)ccc3-c3cccc4oc5ccc6c7ccccc7n(-c7ccccc7)c6c5c34)n2)cc1 ; c1ccc(-c2nc(-c3ccccc3)nc(-c3cc(-c4c(-c5ccccc5)cccc4-c4ccccc4)ccc3-c3cccc4c3oc3c4ccc4c3c3ccccc3n4-c3ccccc3)n2)cc1]

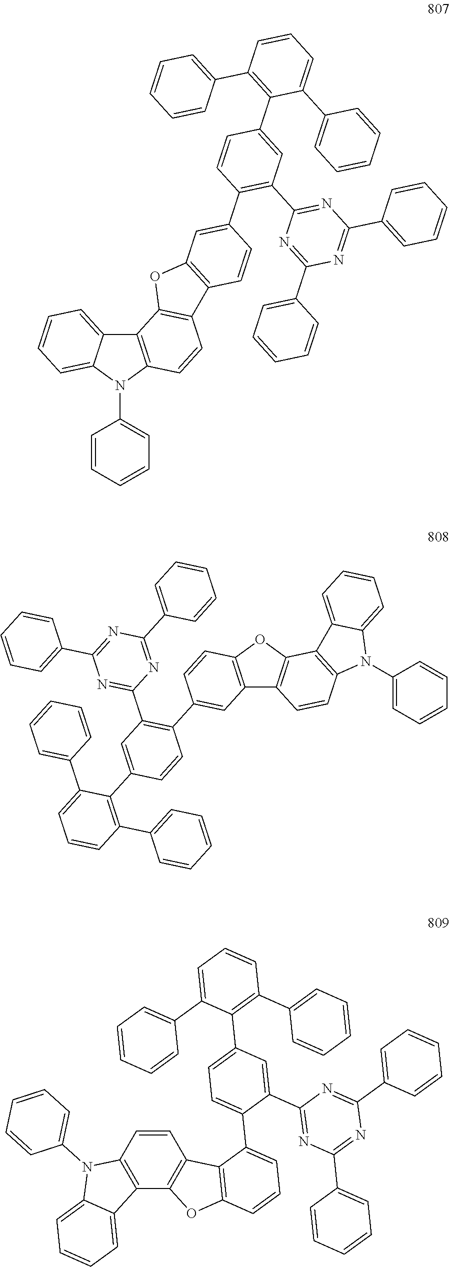 [SMILES: c1ccc(-c2nc(-c3ccccc3)nc(-c3cc(-c4c(-c5ccccc5)cccc4-c4ccccc4)ccc3-c3ccc4c(c3)oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4oc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)ccc3-c3cccc4oc5c(ccc6c5c5ccccc5n6-c5ccccc5)c34)n2)cc1]

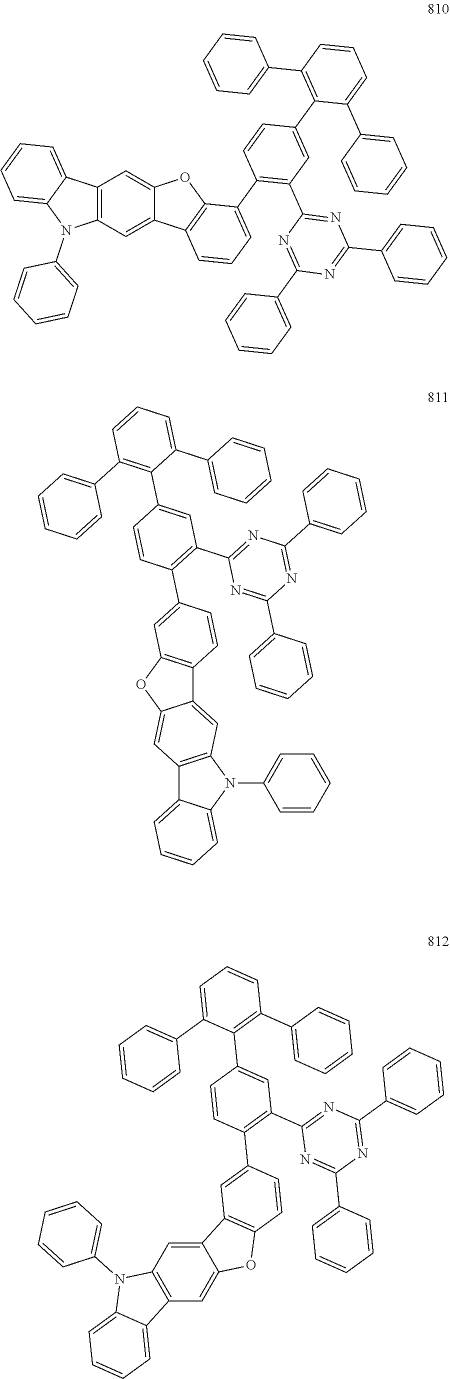 [SMILES: c1ccc(-c2nc(-c3ccccc3)nc(-c3cc(-c4c(-c5ccccc5)cccc4-c4ccccc4)ccc3-c3cccc4c3oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4c(c3)oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4oc5cc6c7ccccc7n(-c7ccccc7)c6cc5c4c3)n2)cc1]

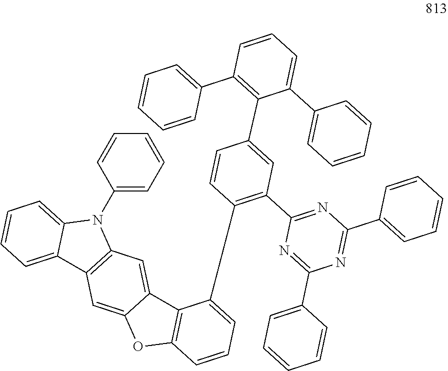 [SMILES: c1ccc(-c2nc(-c3ccccc3)nc(-c3cc(-c4c(-c5ccccc5)cccc4-c4ccccc4)ccc3-c3cccc4oc5cc6c7ccccc7n(-c7ccccc7)c6cc5c34)n2)cc1]

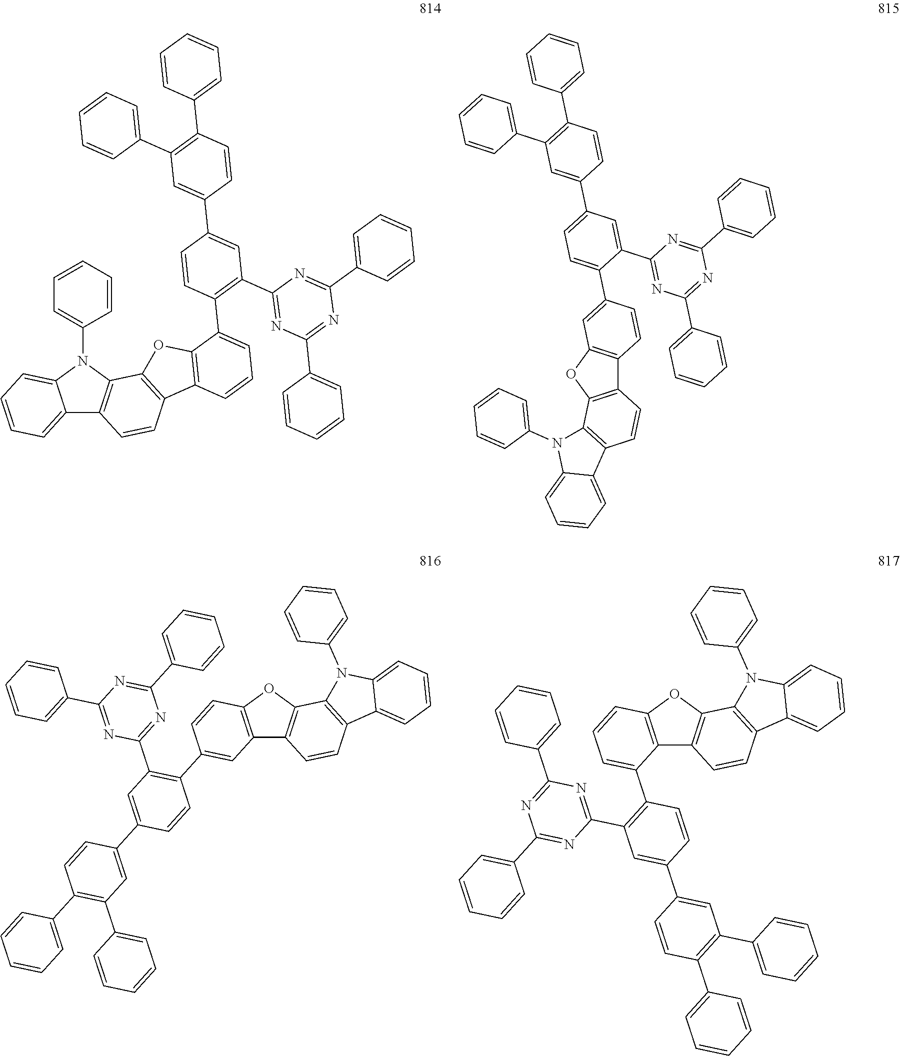 [SMILES: c1ccc(-c2nc(-c3ccccc3)nc(-c3cc(-c4ccc(-c5ccccc5)c(-c5ccccc5)c4)ccc3-c3cccc4c3oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4c(c3)oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)ccc3-c3cccc4oc5c(ccc6c7ccccc7n(-c7ccccc7)c65)c34)n2)cc1]

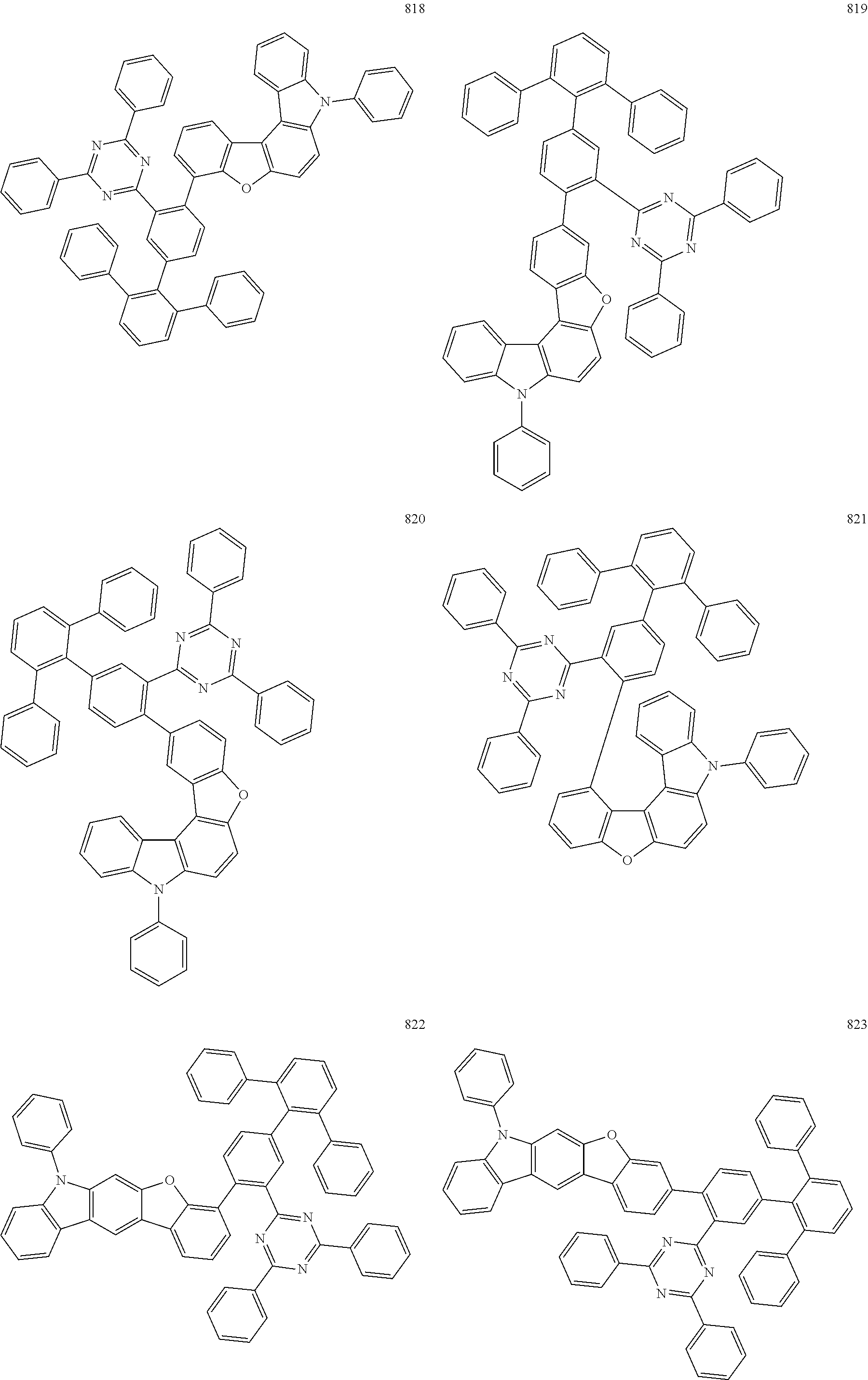

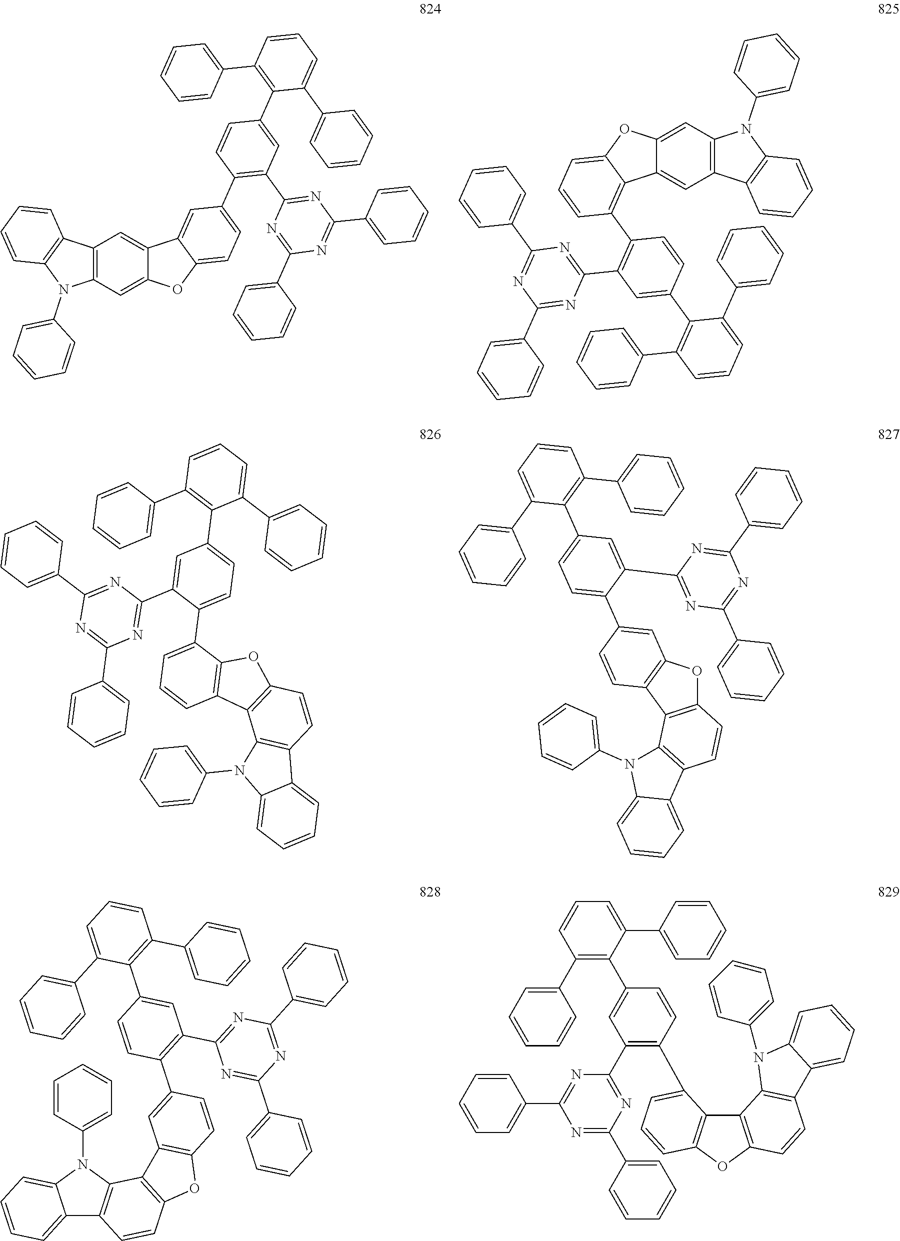

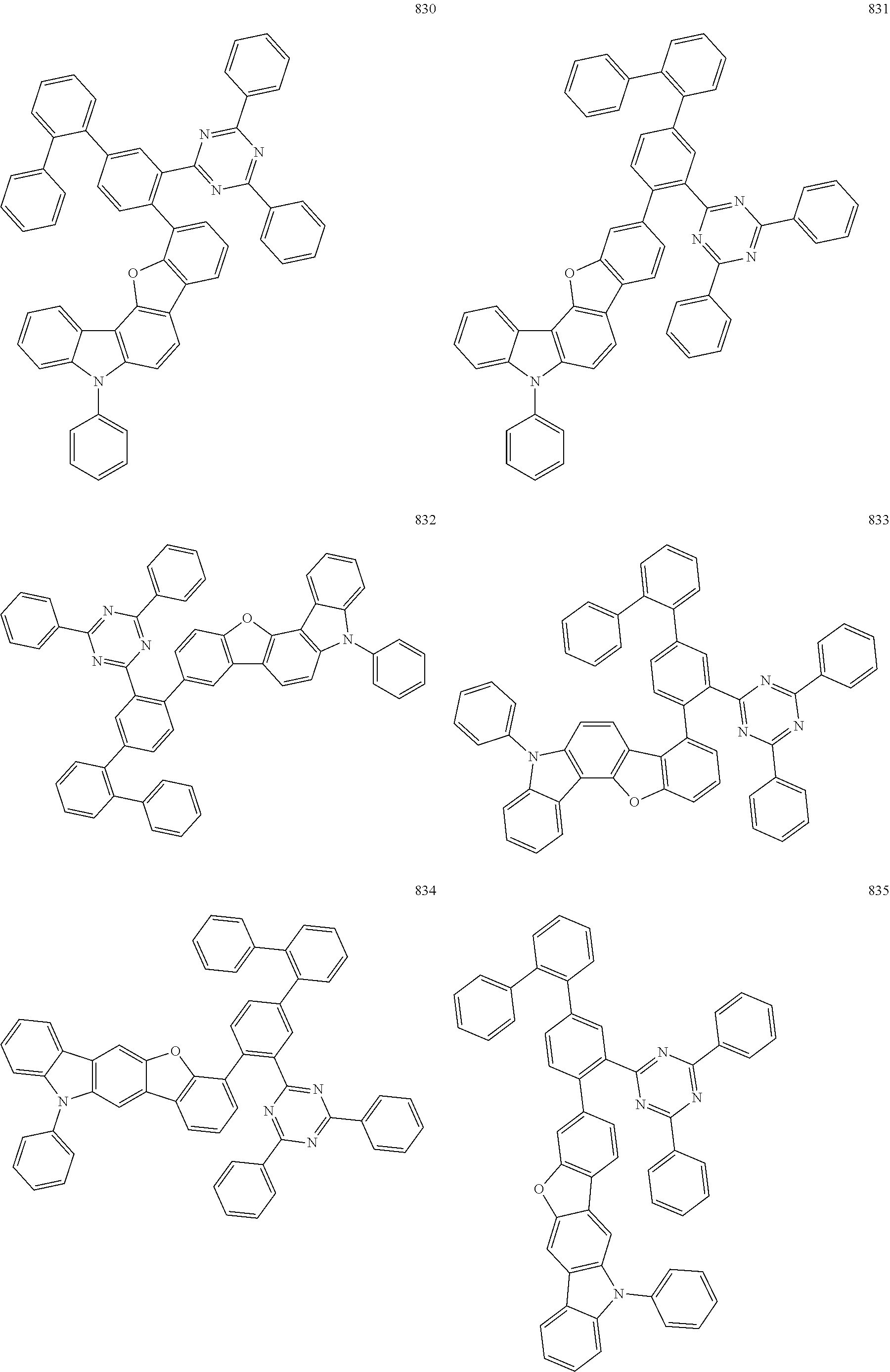

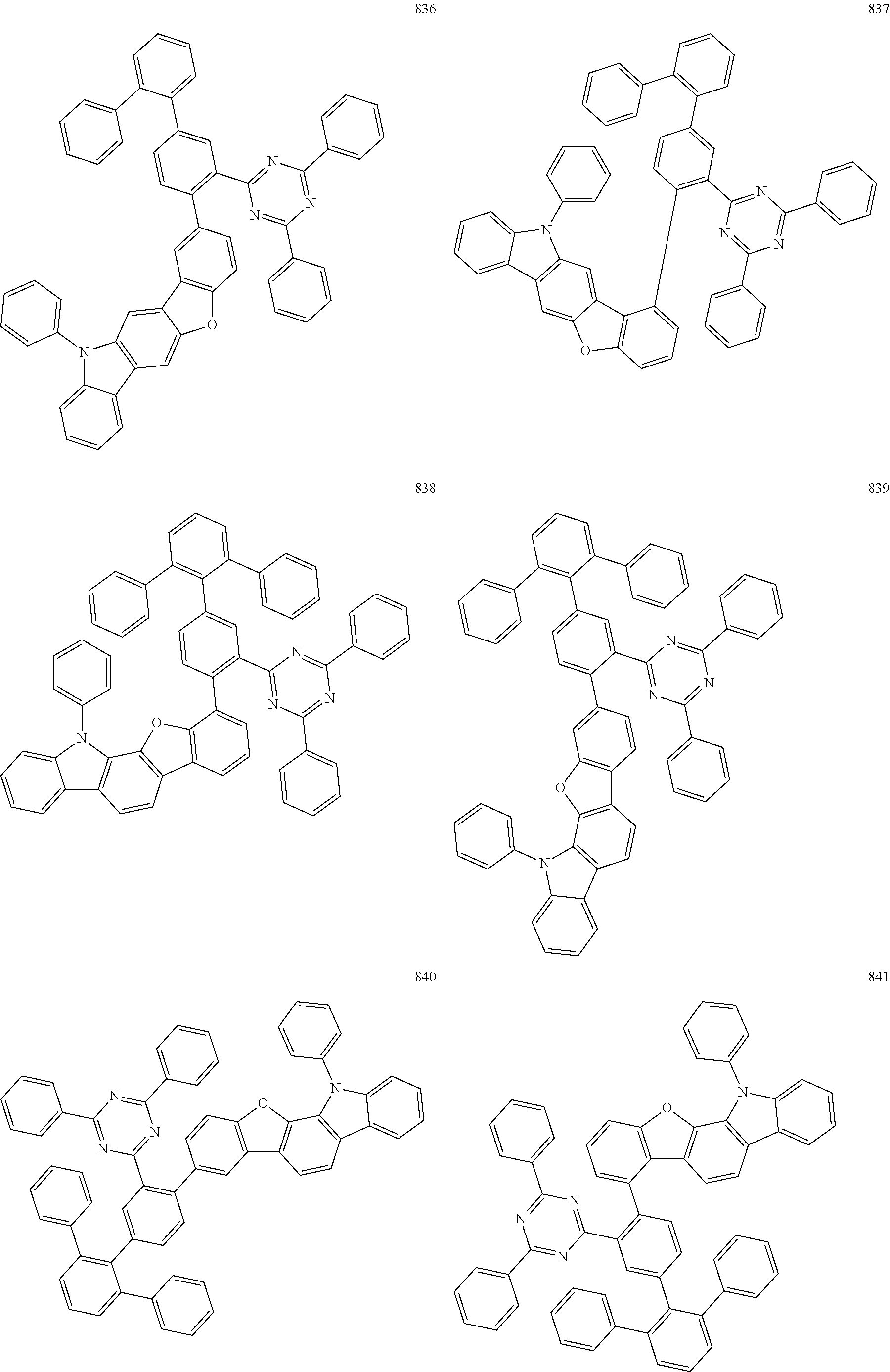

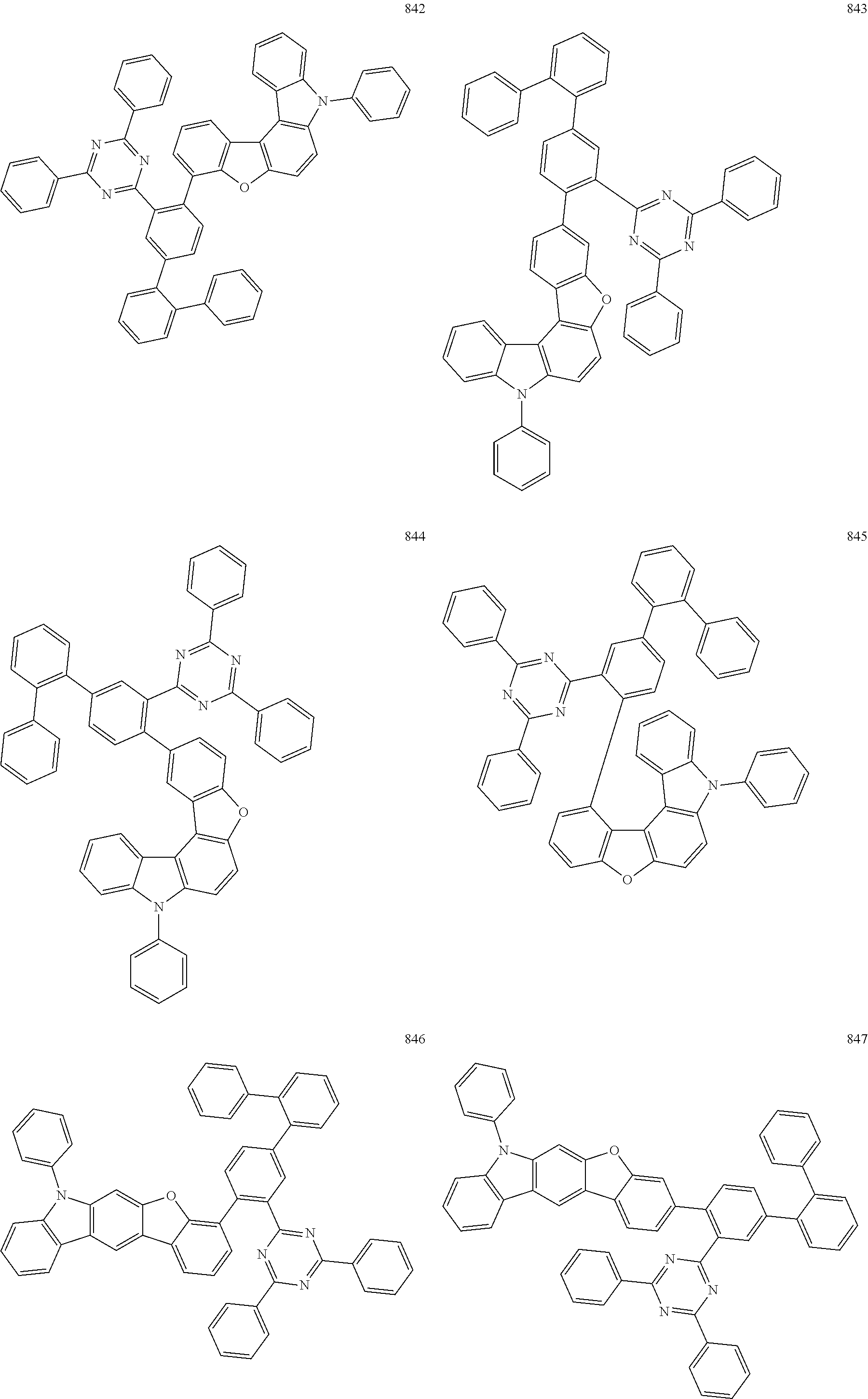

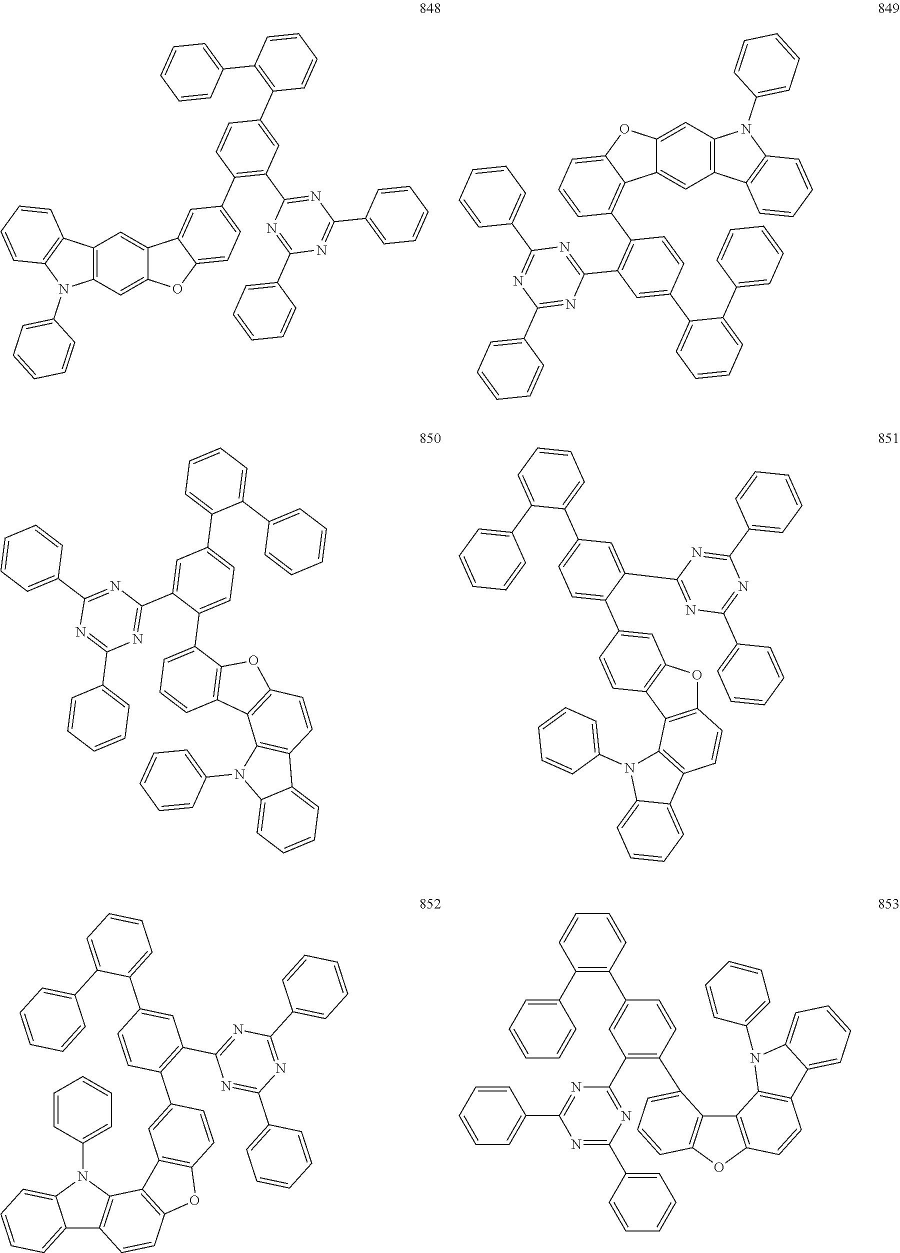

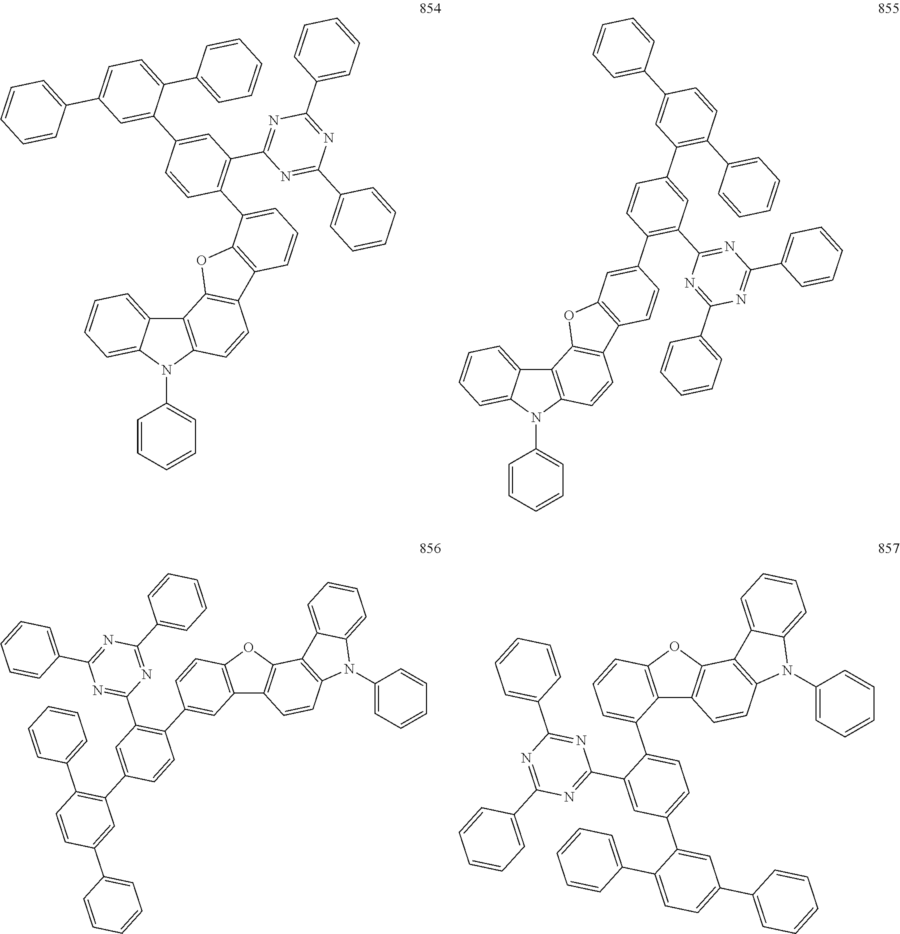 [SMILES: c1ccc(-c2ccc(-c3ccccc3)c(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6c(ccc7c6c6ccccc6n7-c6ccccc6)c45)c(-c4nc(-c5ccccc5)nc(-c5ccccc5)n4)c3)c2)cc1]

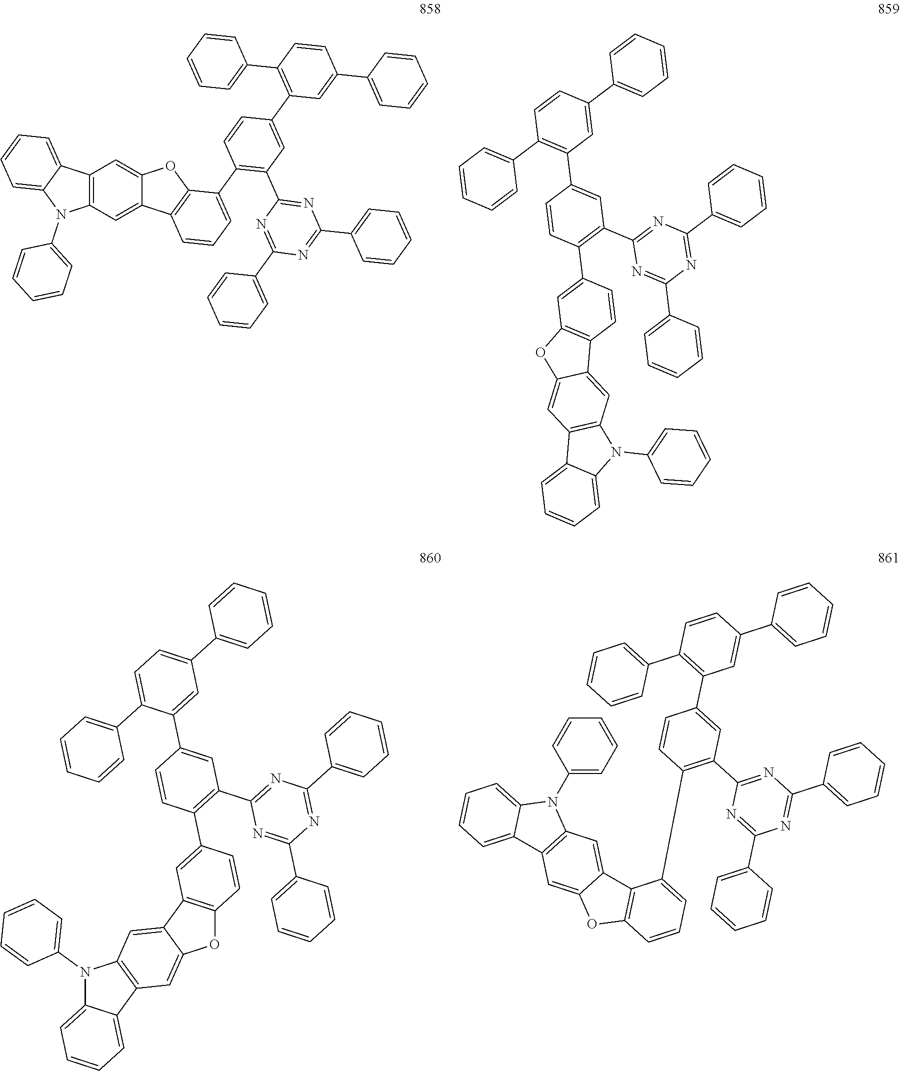 [SMILES: c1ccc(-c2ccc(-c3ccccc3)c(-c3ccc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6cc7c8ccccc8n(-c8ccccc8)c7cc6c45)c(-c4nc(-c5ccccc5)nc(-c5ccccc5)n4)c3)c2)cc1]

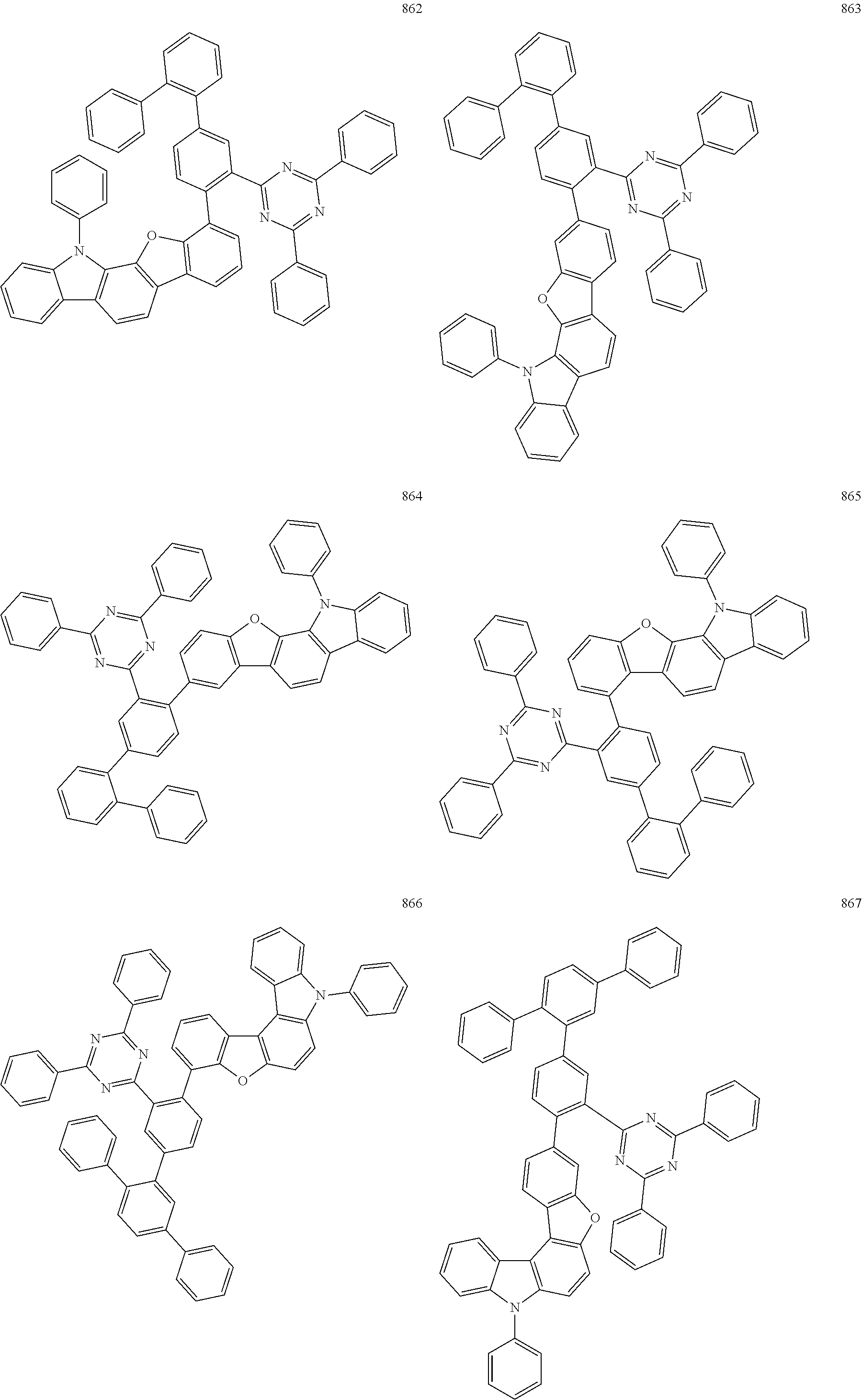

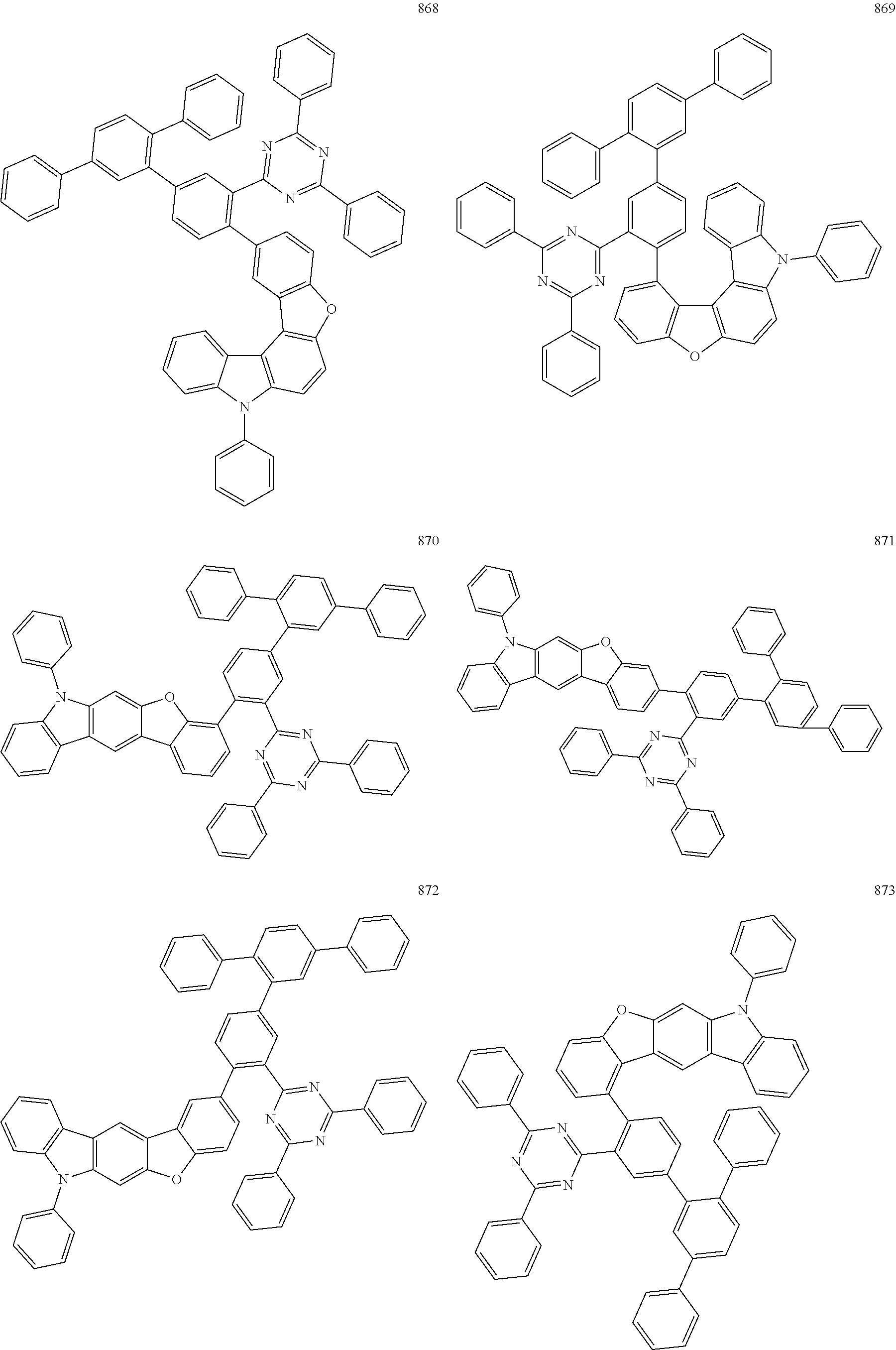

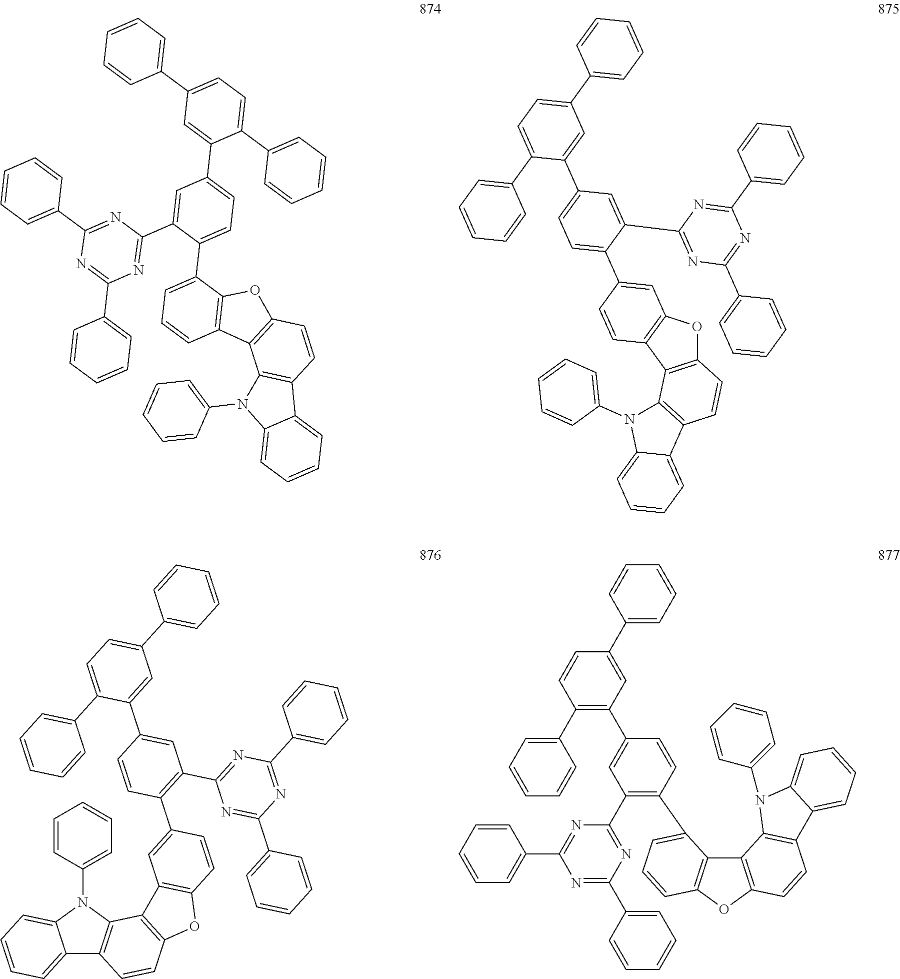 [SMILES: c1ccc(-c2ccc(-c3ccccc3)c(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6ccc7c8ccccc8n(-c8ccccc8)c7c6c45)c(-c4nc(-c5ccccc5)nc(-c5ccccc5)n4)c3)c2)cc1]

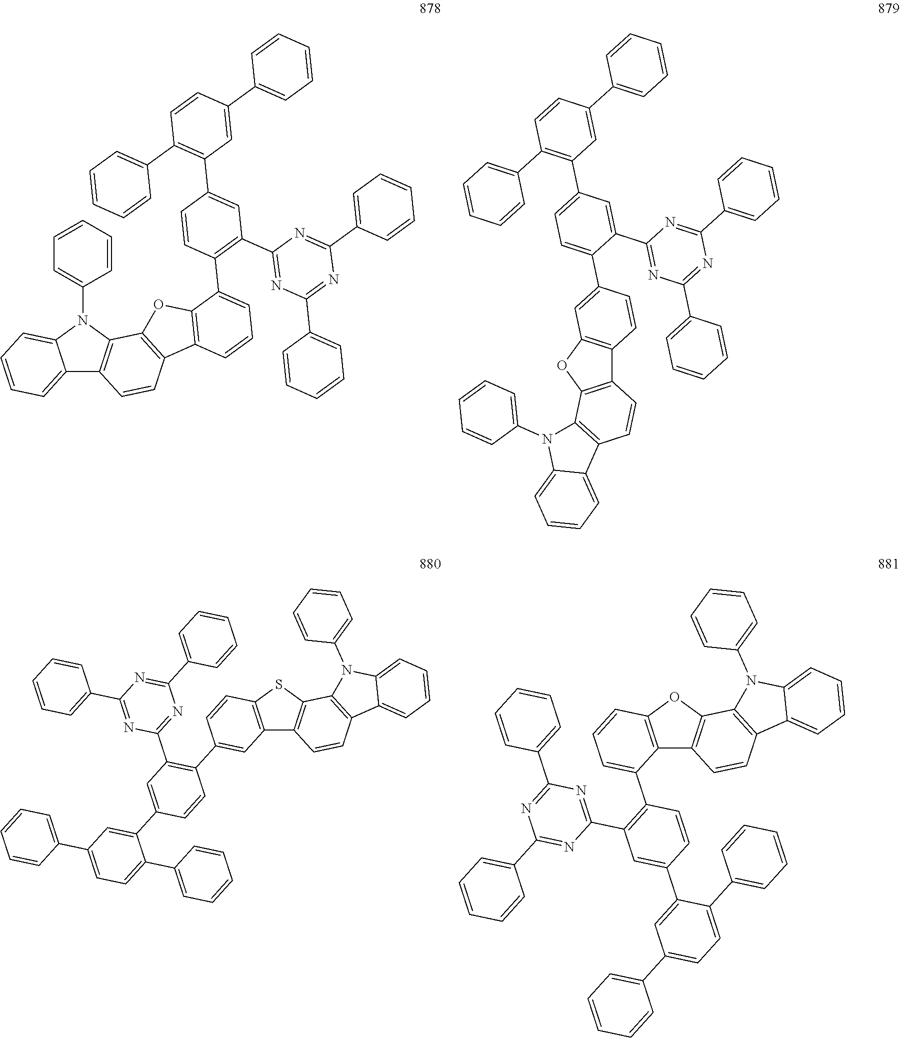 [SMILES: c1ccc(-c2ccc(-c3ccccc3)c(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6c(ccc7c8ccccc8n(-c8ccccc8)c76)c45)c(-c4nc(-c5ccccc5)nc(-c5ccccc5)n4)c3)c2)cc1]

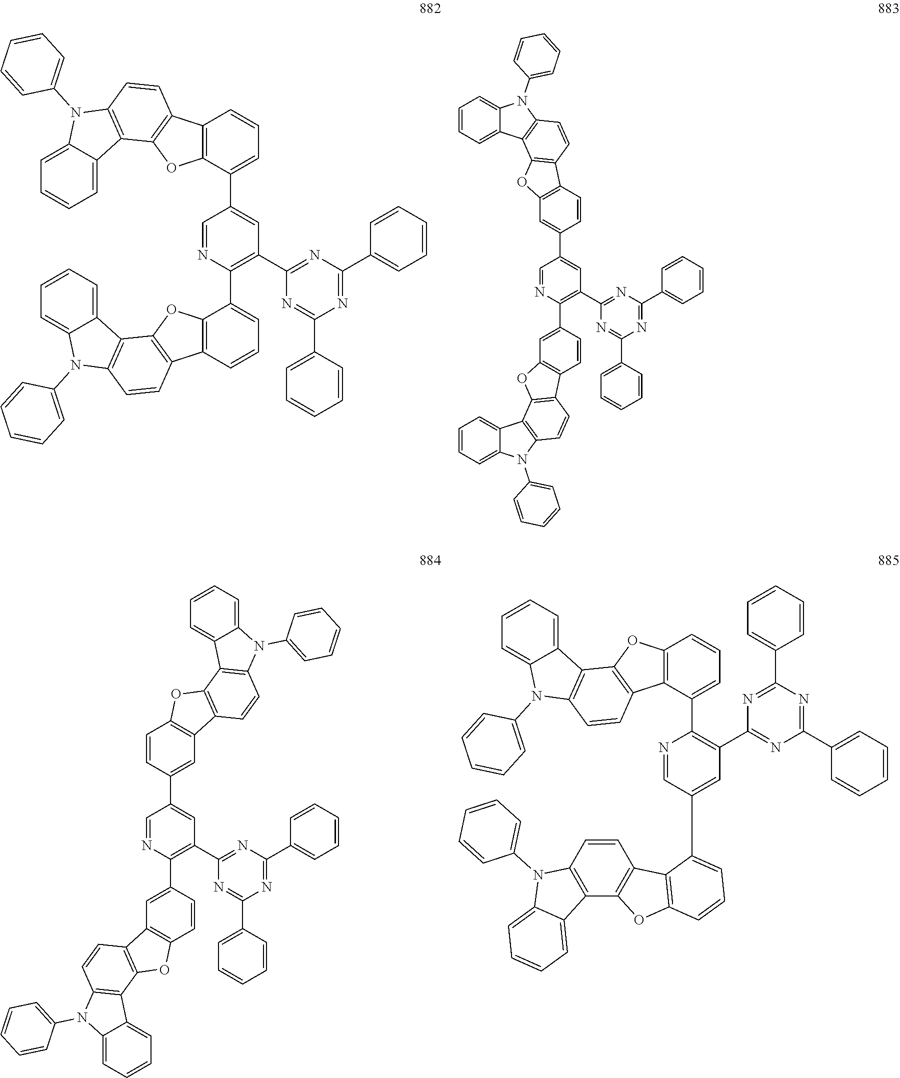

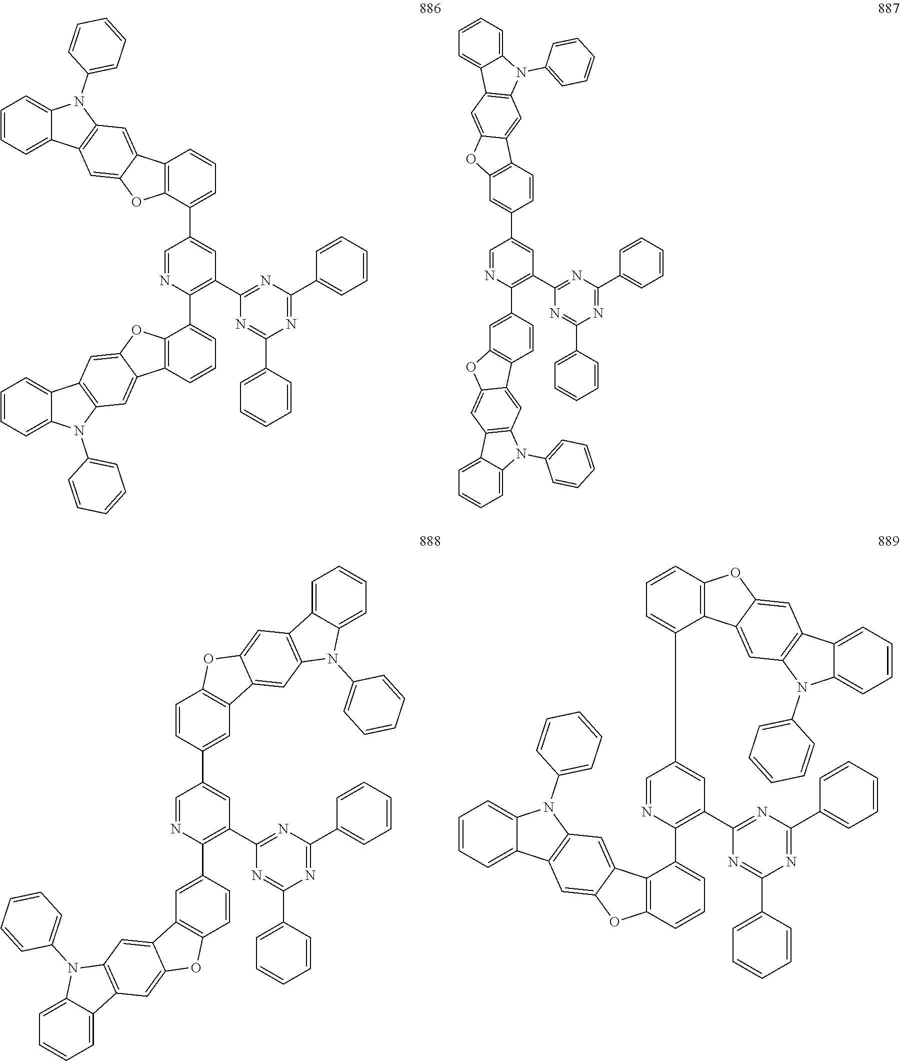

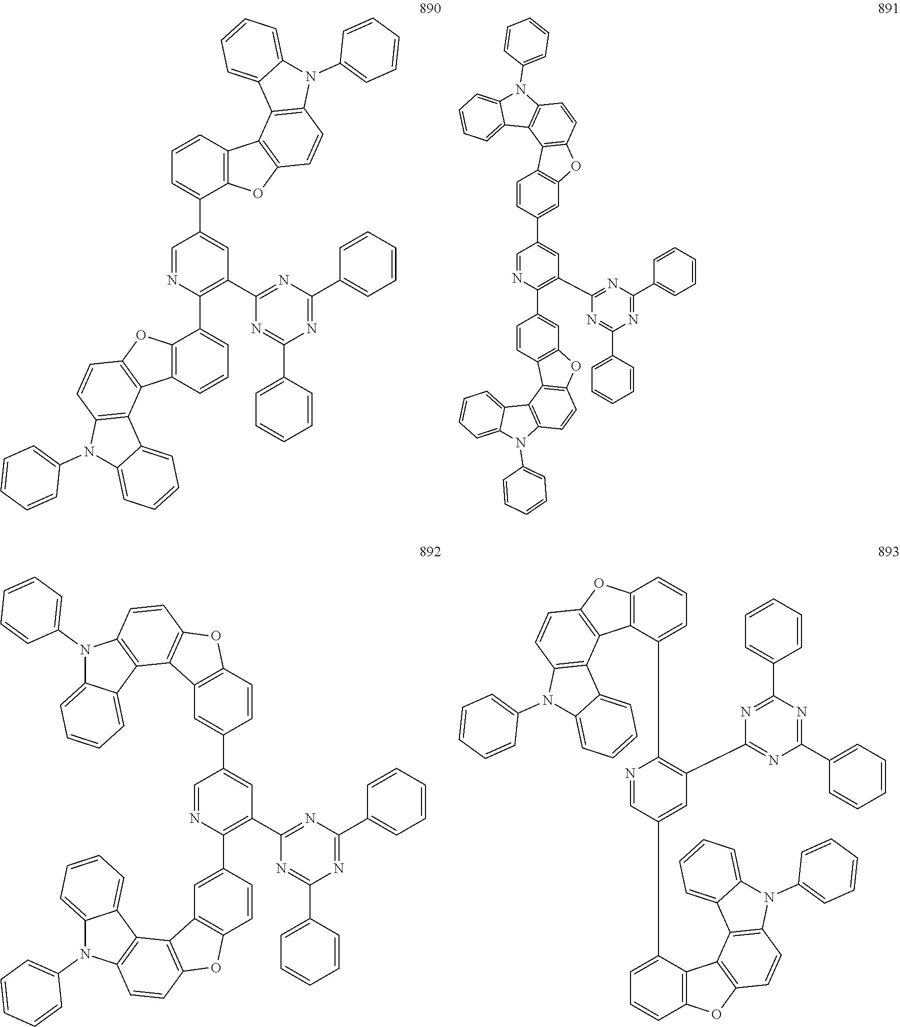

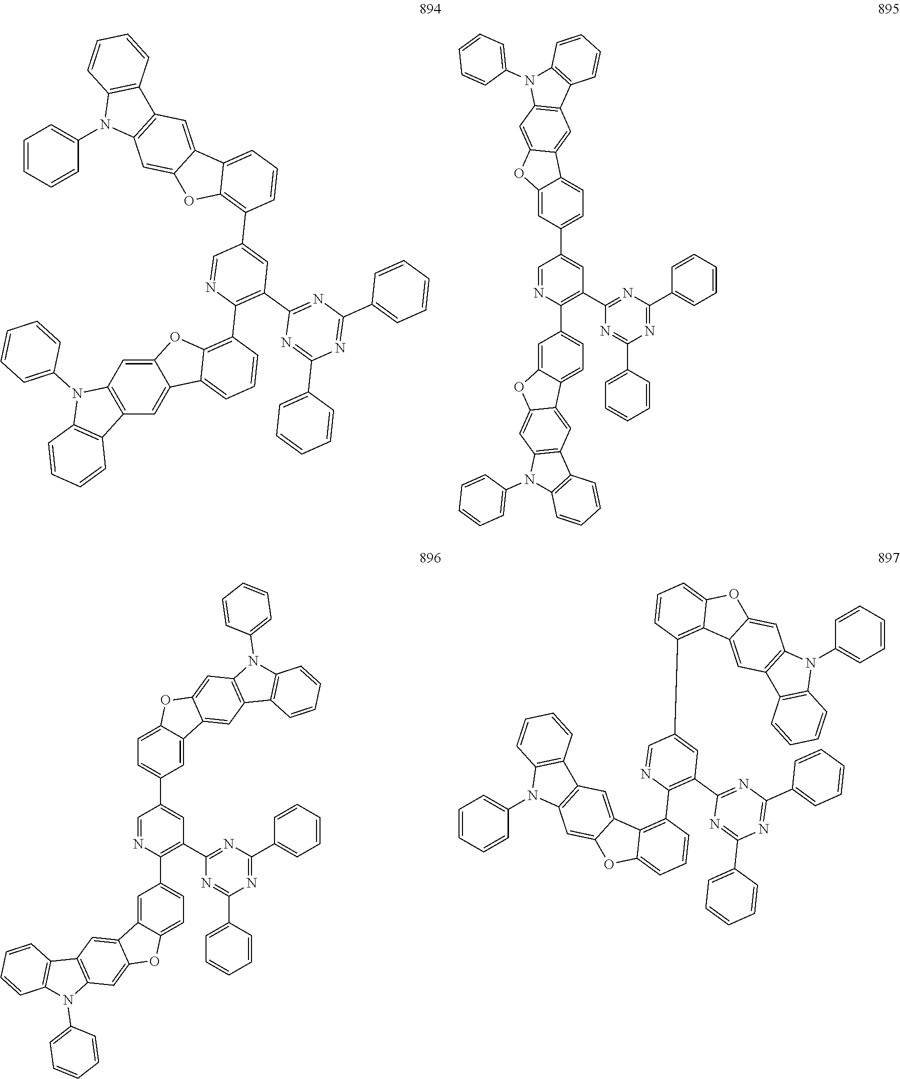

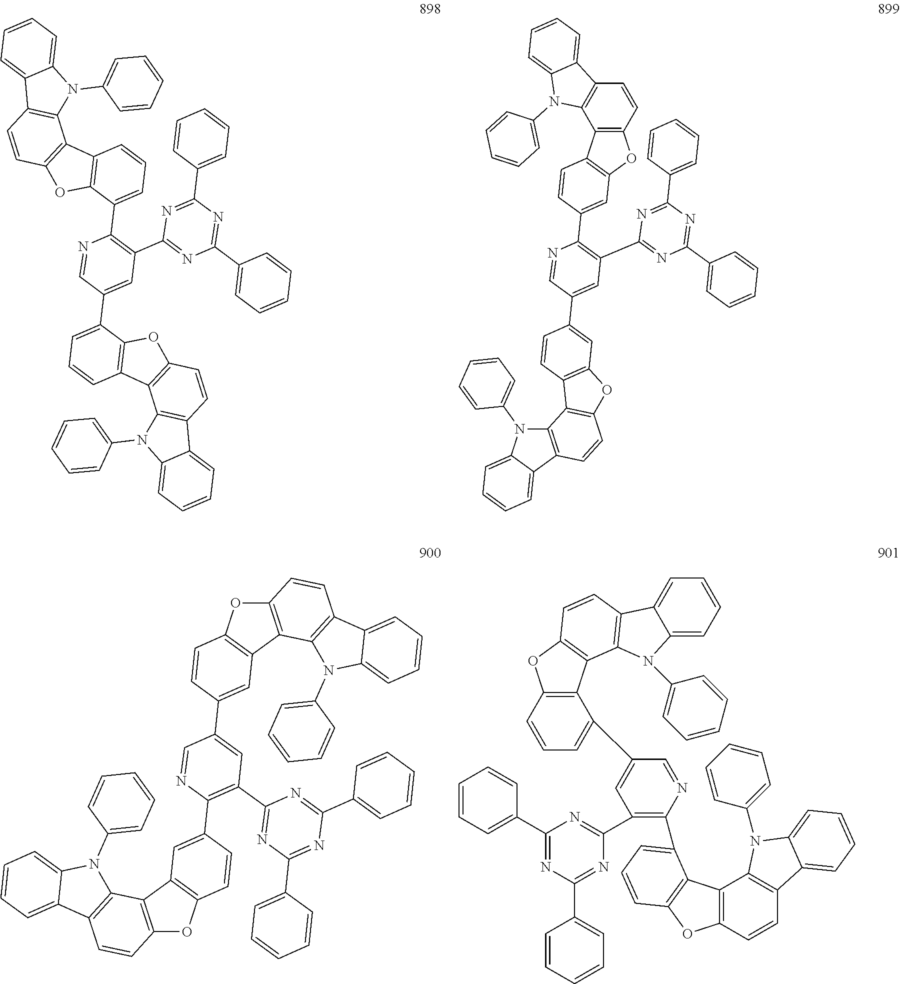

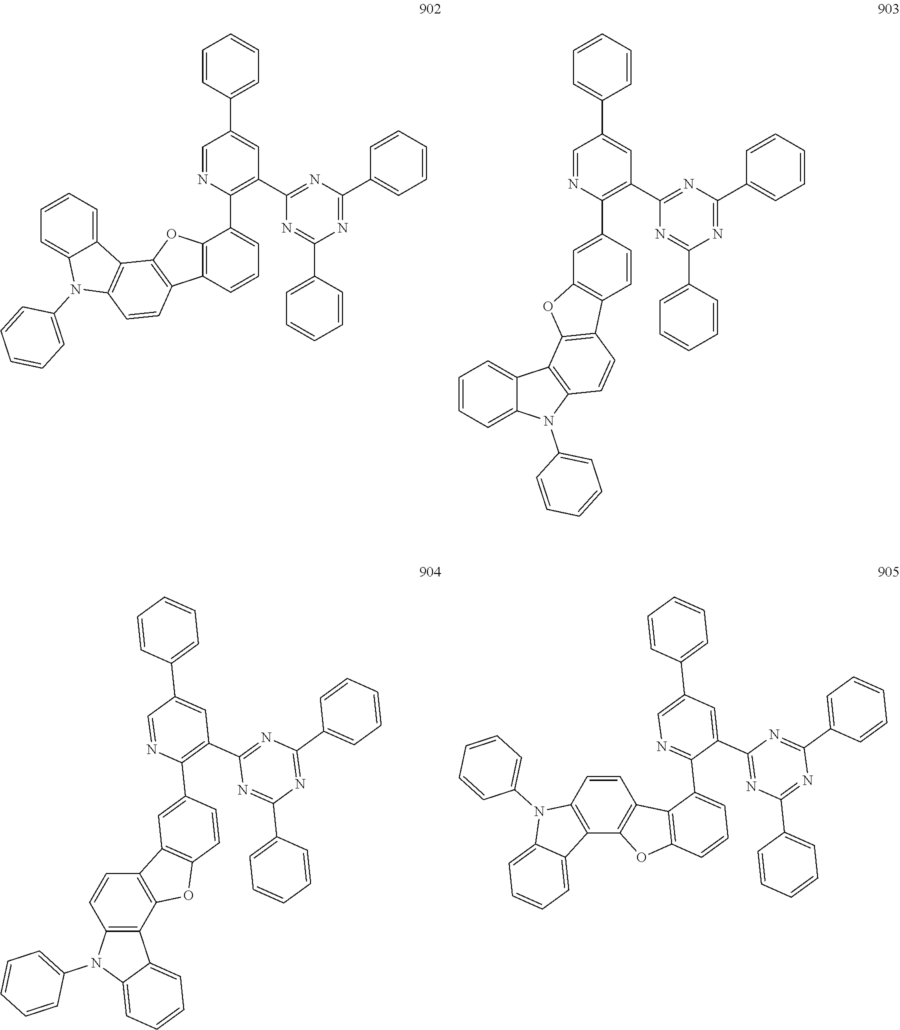 [SMILES: c1ccc(-c2cnc(-c3cccc4c3oc3c4ccc4c3c3ccccc3n4-c3ccccc3)c(-c3nc(-c4ccccc4)nc(-c4ccccc4)n3)c2)cc1 ; c1ccc(-c2cnc(-c3ccc4c(c3)oc3c4ccc4c3c3ccccc3n4-c3ccccc3)c(-c3nc(-c4ccccc4)nc(-c4ccccc4)n3)c2)cc1 ; c1ccc(-c2cnc(-c3ccc4oc5c(ccc6c5c5ccccc5n6-c5ccccc5)c4c3)c(-c3nc(-c4ccccc4)nc(-c4ccccc4)n3)c2)cc1 ; c1ccc(-c2cnc(-c3cccc4oc5c(ccc6c5c5ccccc5n6-c5ccccc5)c34)c(-c3nc(-c4ccccc4)nc(-c4ccccc4)n3)c2)cc1]

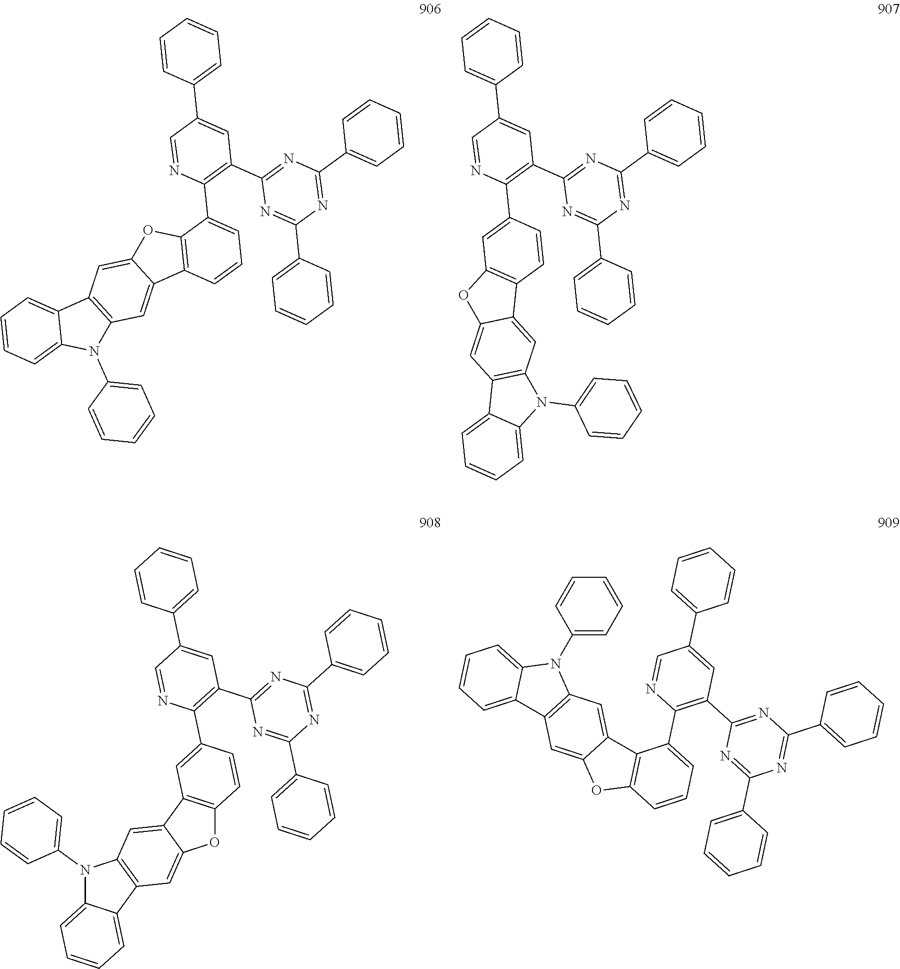 [SMILES: c1ccc(-c2cnc(-c3cccc4c3oc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4c(c3)oc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4oc5cc6c7ccccc7n(-c7ccccc7)c6cc5c4c3)c(-c3nc(-c4ccccc4)nc(-c4ccccc4)n3)c2)cc1 ; c1ccc(-c2cnc(-c3cccc4oc5cc6c7ccccc7n(-c7ccccc7)c6cc5c34)c(-c3nc(-c4ccccc4)nc(-c4ccccc4)n3)c2)cc1]

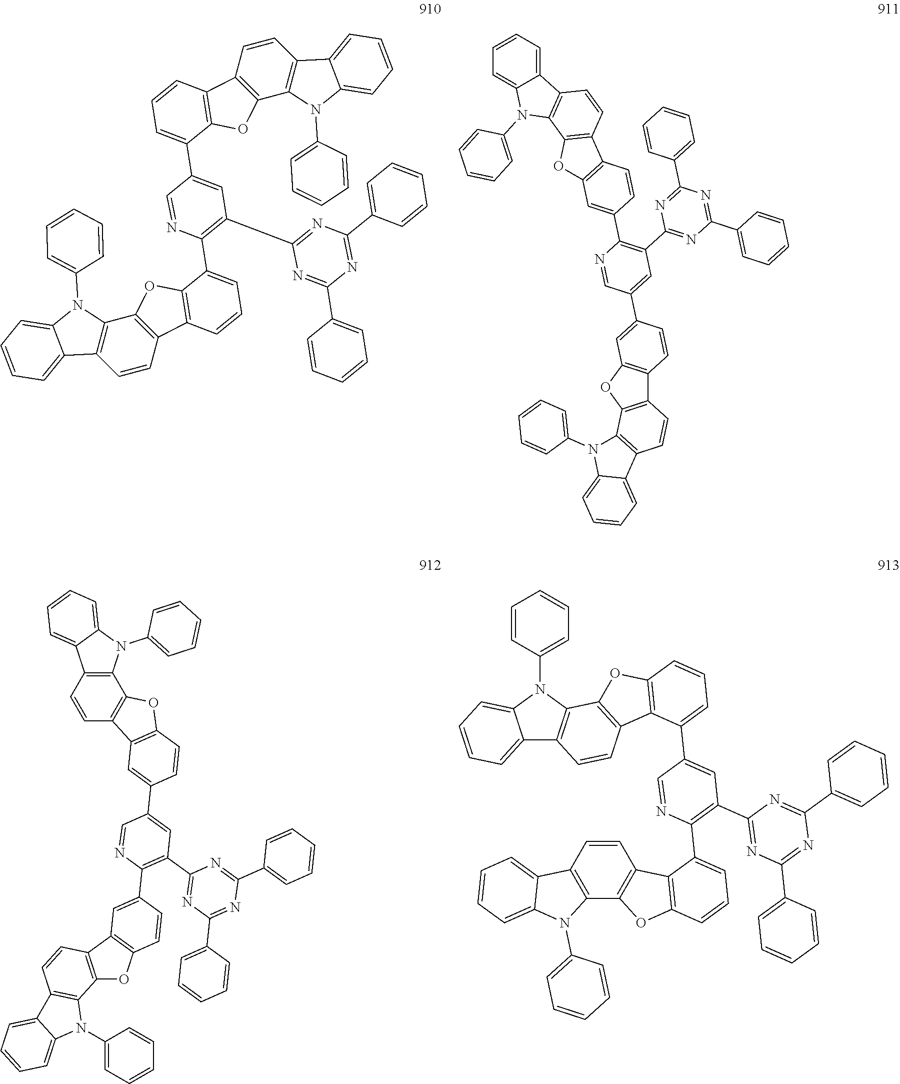

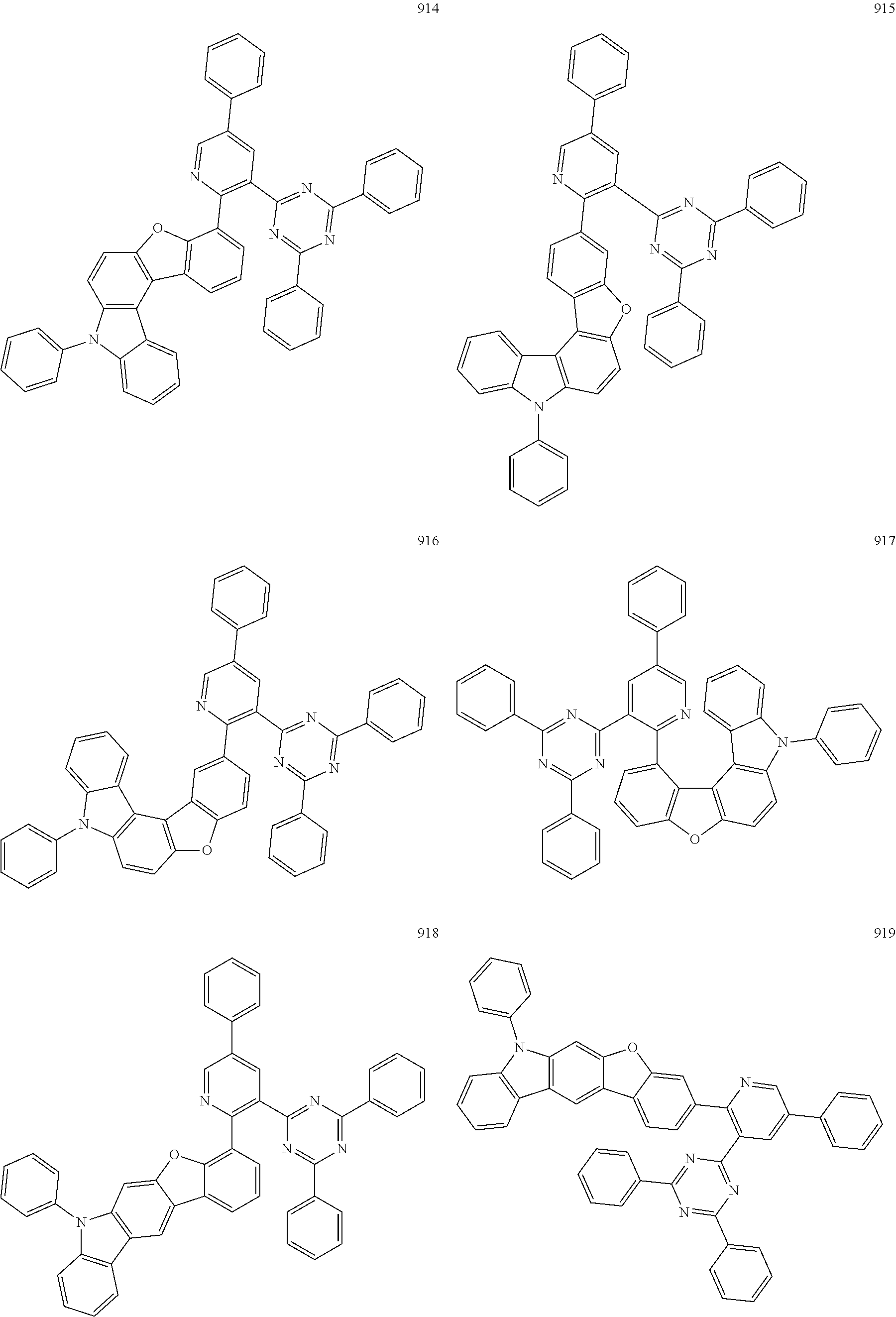

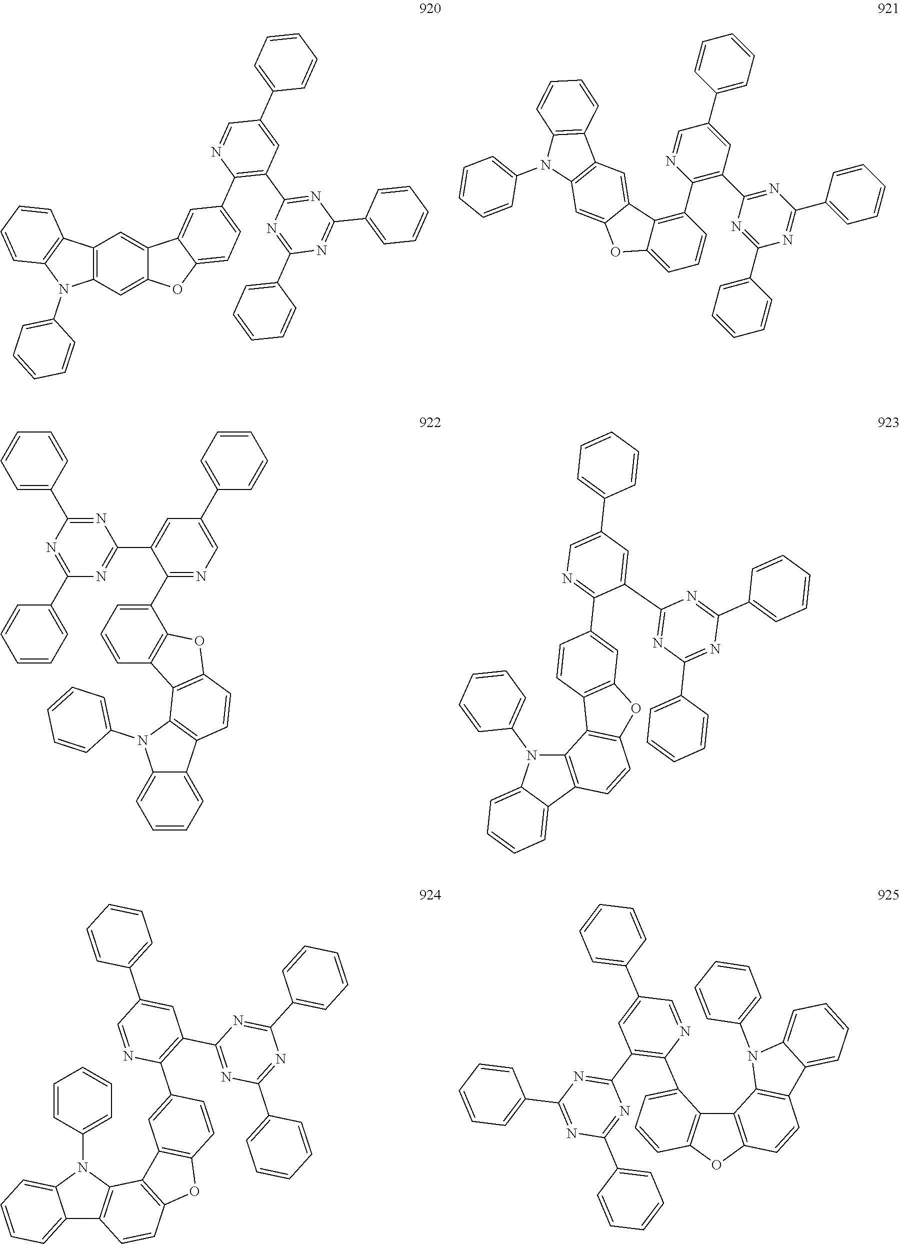

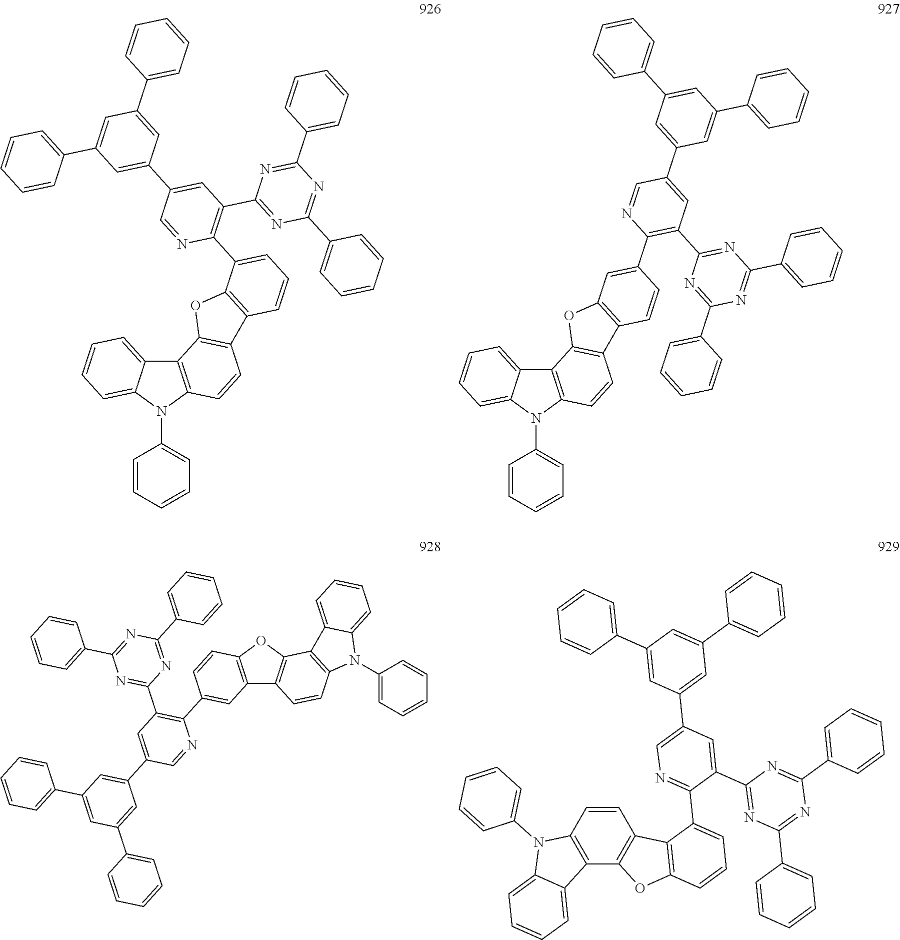 [SMILES: c1ccc(-c2cc(-c3ccccc3)cc(-c3cnc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6c(ccc7c6c6ccccc6n7-c6ccccc6)c45)c(-c4nc(-c5ccccc5)nc(-c5ccccc5)n4)c3)c2)cc1]

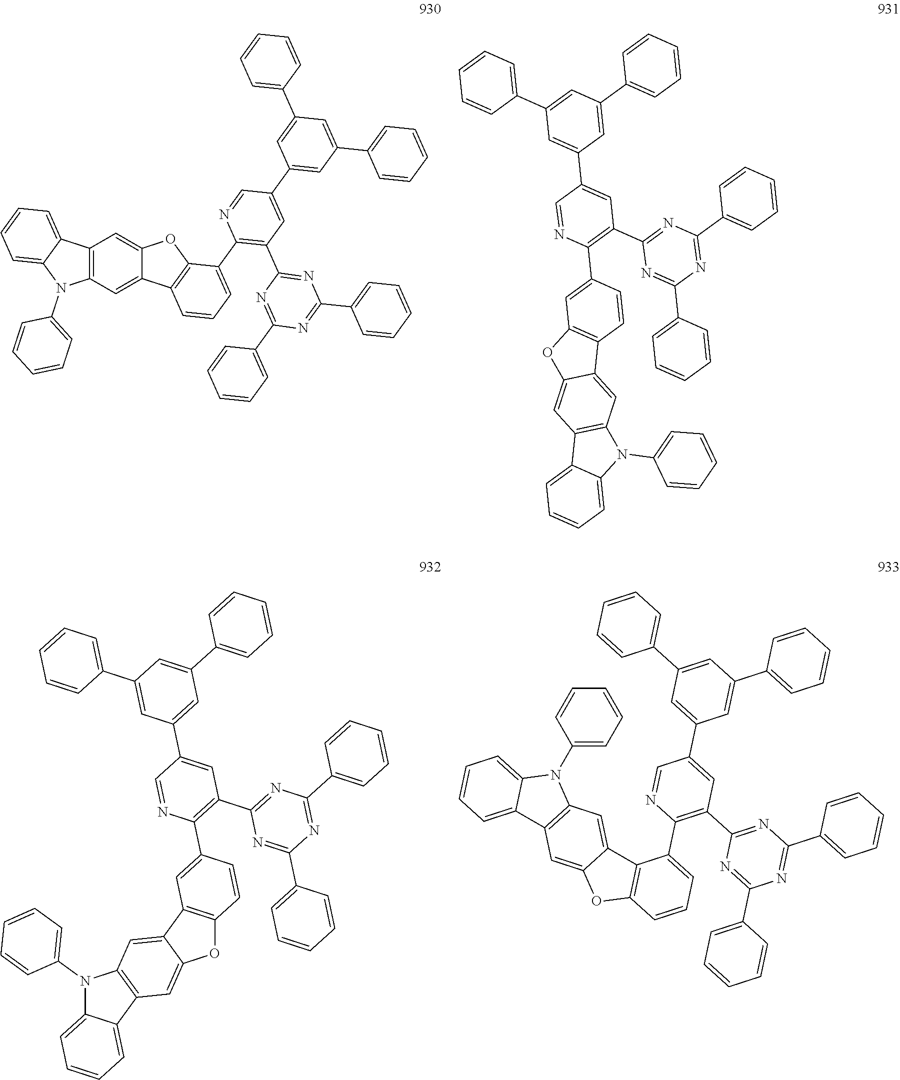 [SMILES: c1ccc(-c2cc(-c3ccccc3)cc(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

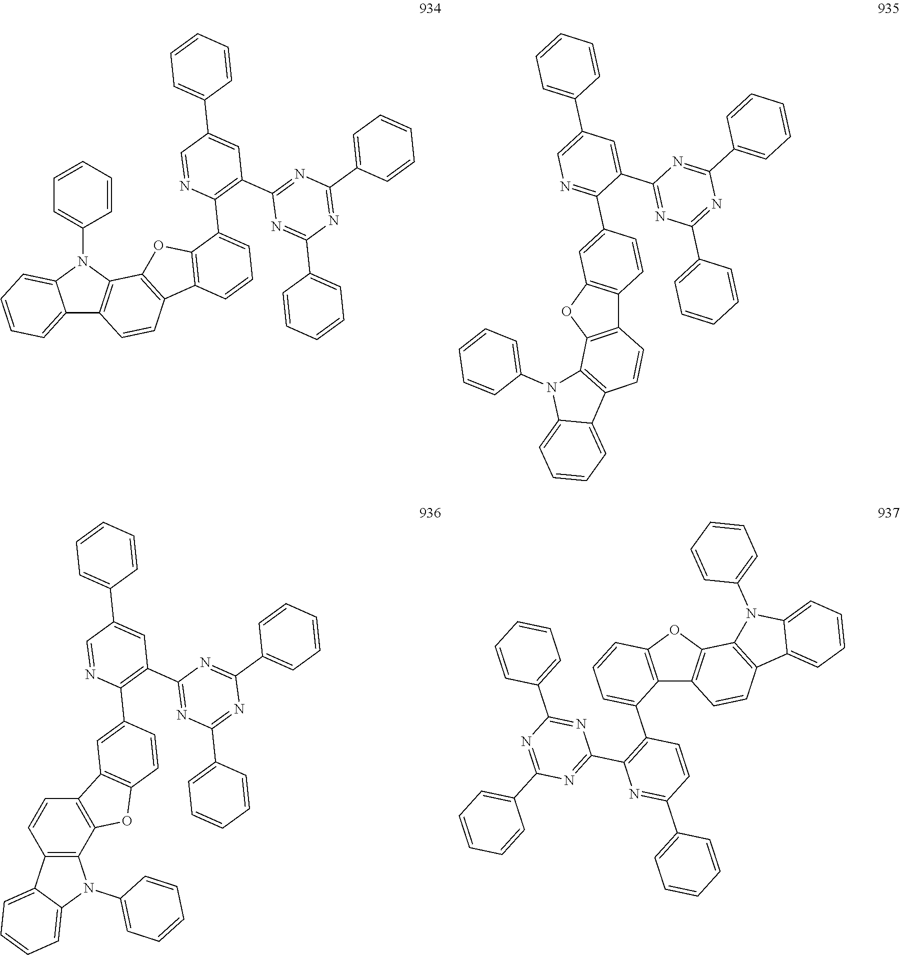 [SMILES: c1ccc(-c2cnc(-c3cccc4c3oc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4c(c3)oc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4oc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4oc5c(ccc6c7ccccc7n(-c7ccccc7)c65)c34)c(-c3nc(-c4ccccc4)nc(-c4ccccc4)n3)n2)cc1]

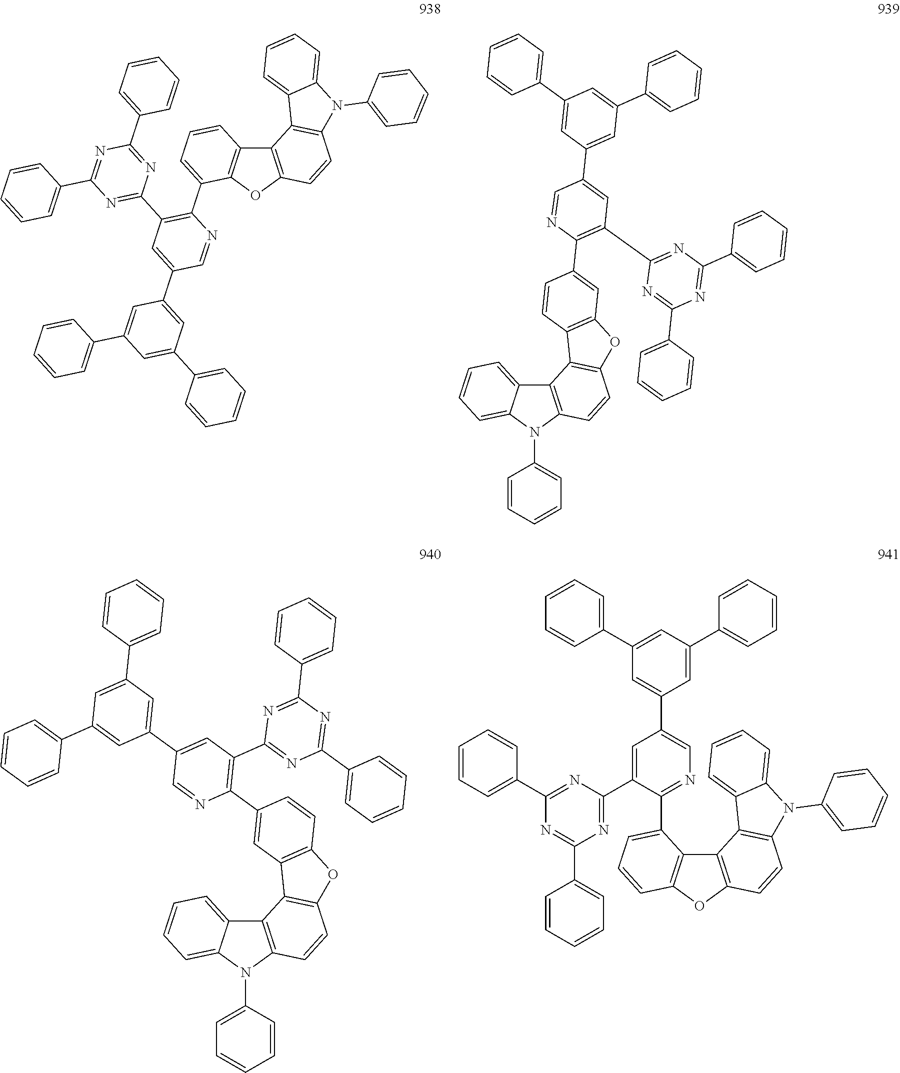 [SMILES: c1ccc(-c2cc(-c3ccccc3)cc(-c3cnc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6ccc7c(c8ccccc8n7-c7ccccc7)c6c45)c(-c4nc(-c5ccccc5)nc(-c5ccccc5)n4)c3)c2)cc1]

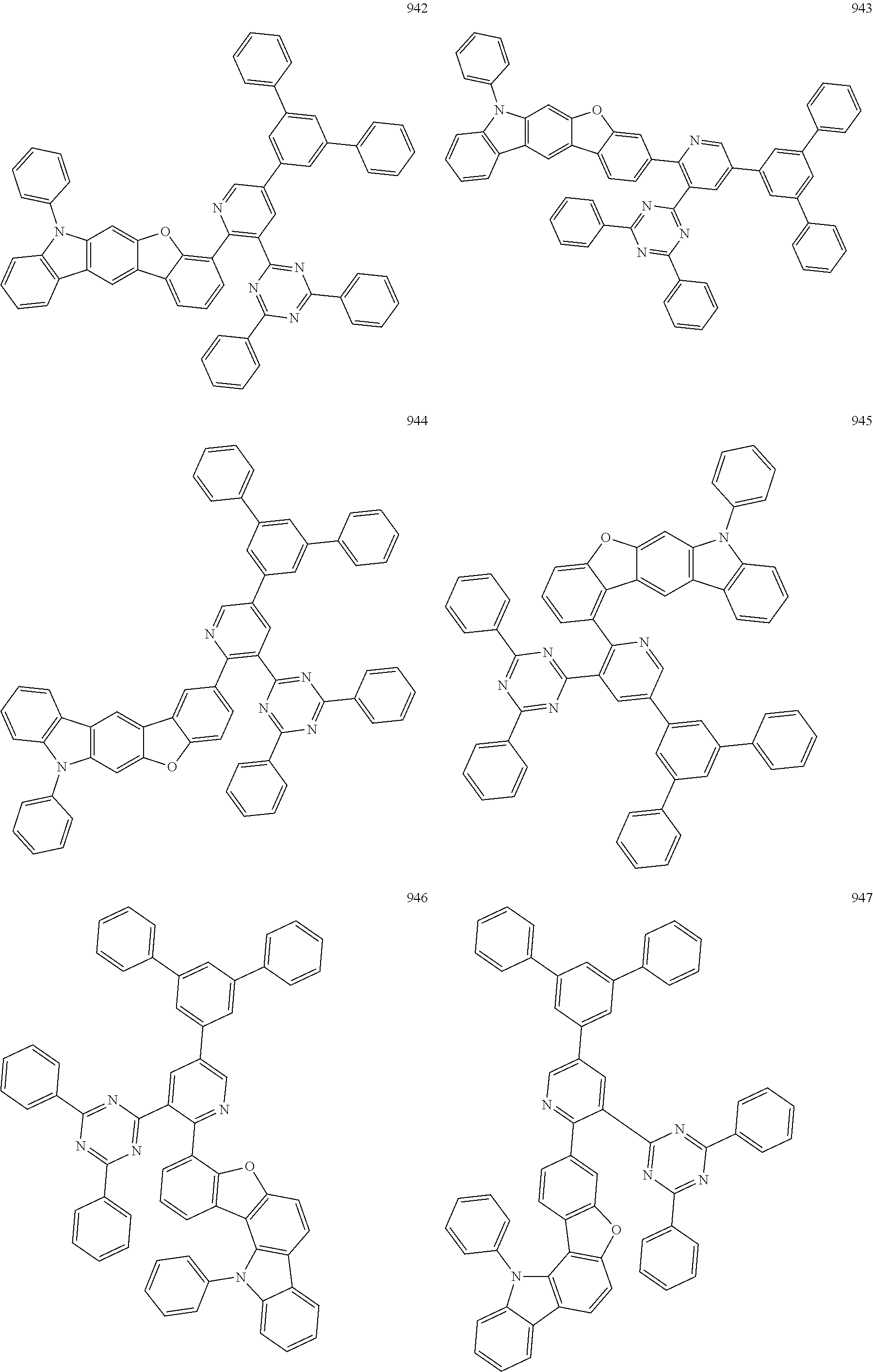

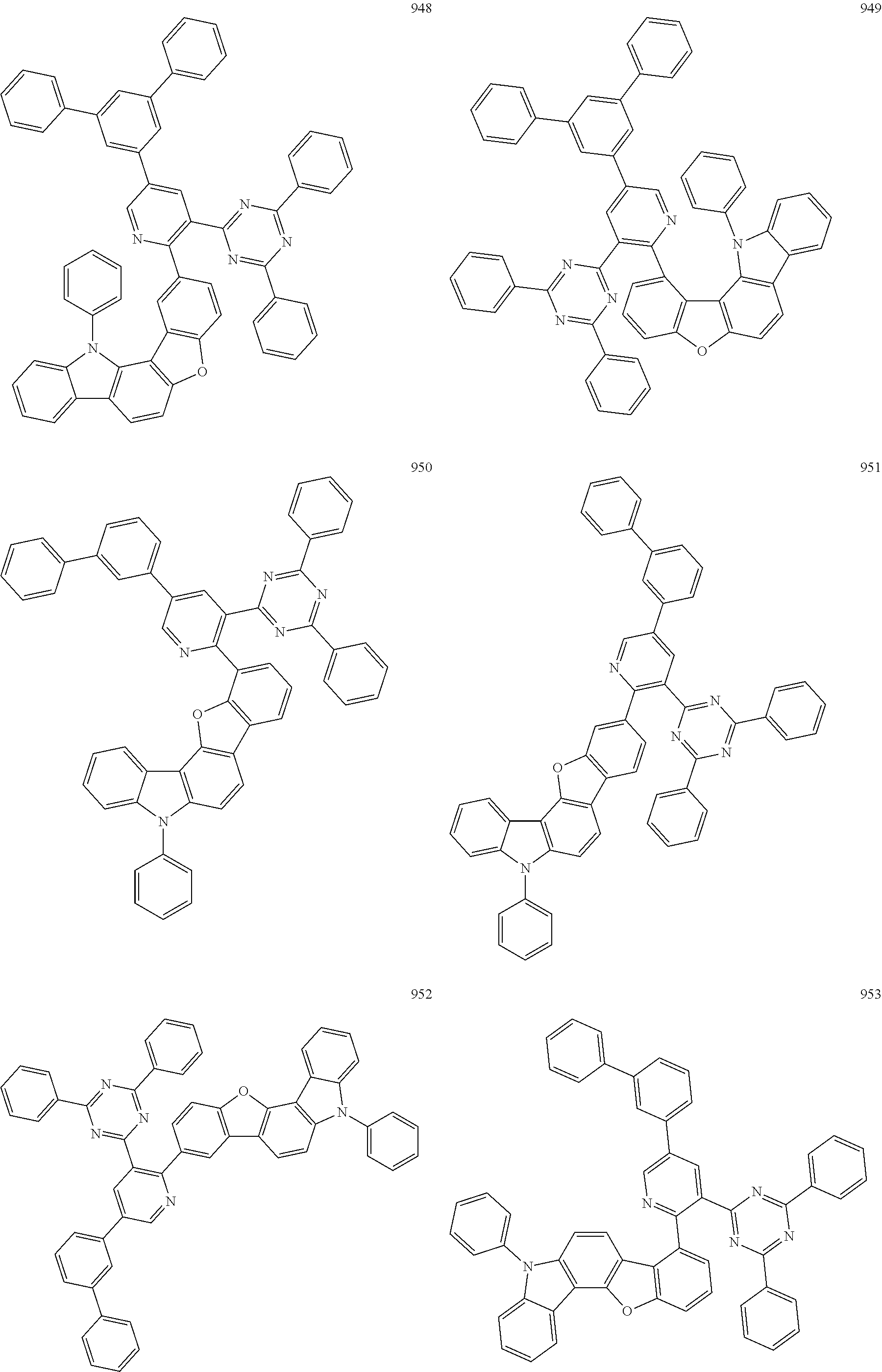

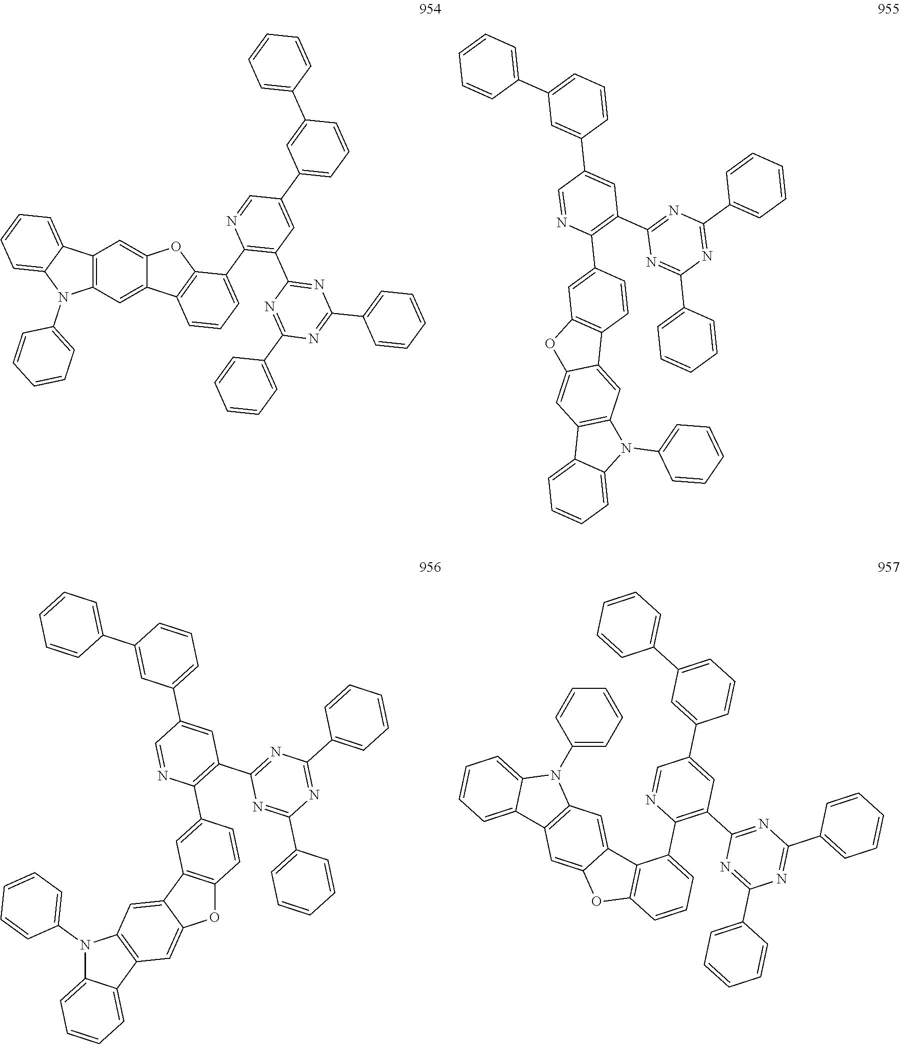 [SMILES: c1ccc(-c2cccc(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

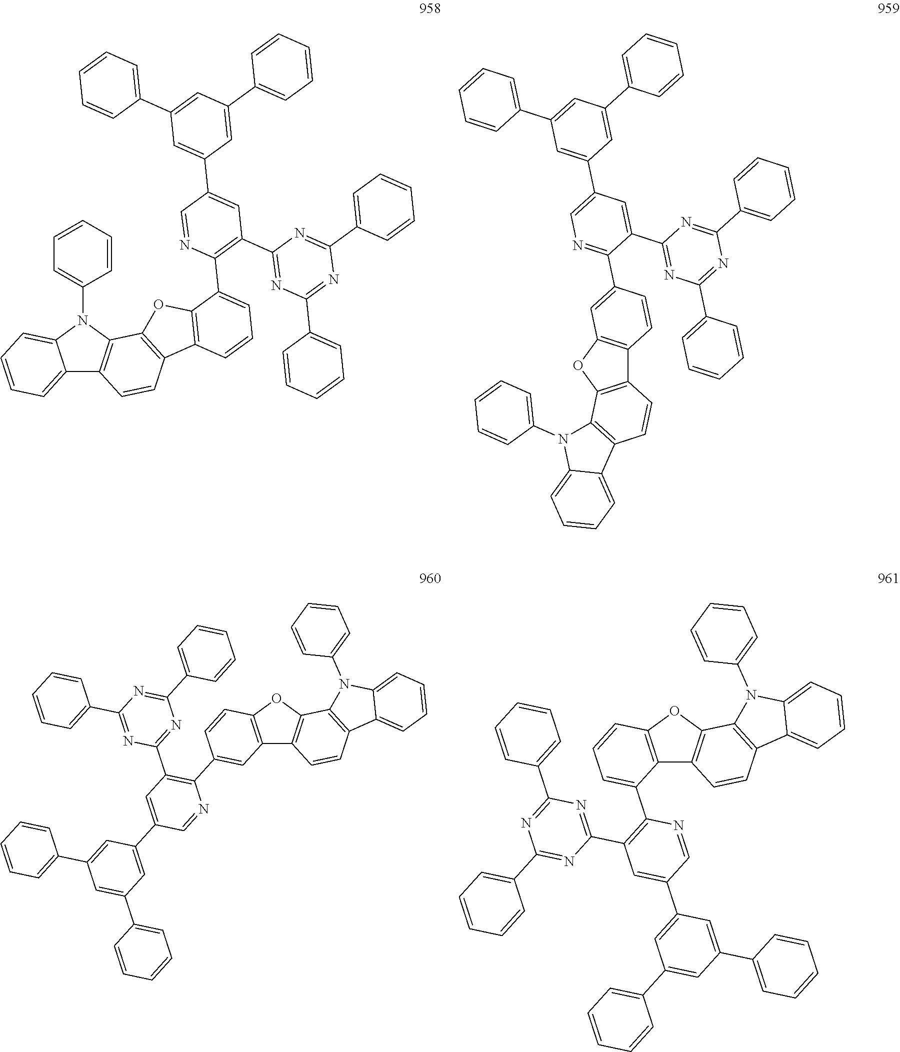 [SMILES: c1ccc(-c2cc(-c3ccccc3)cc(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

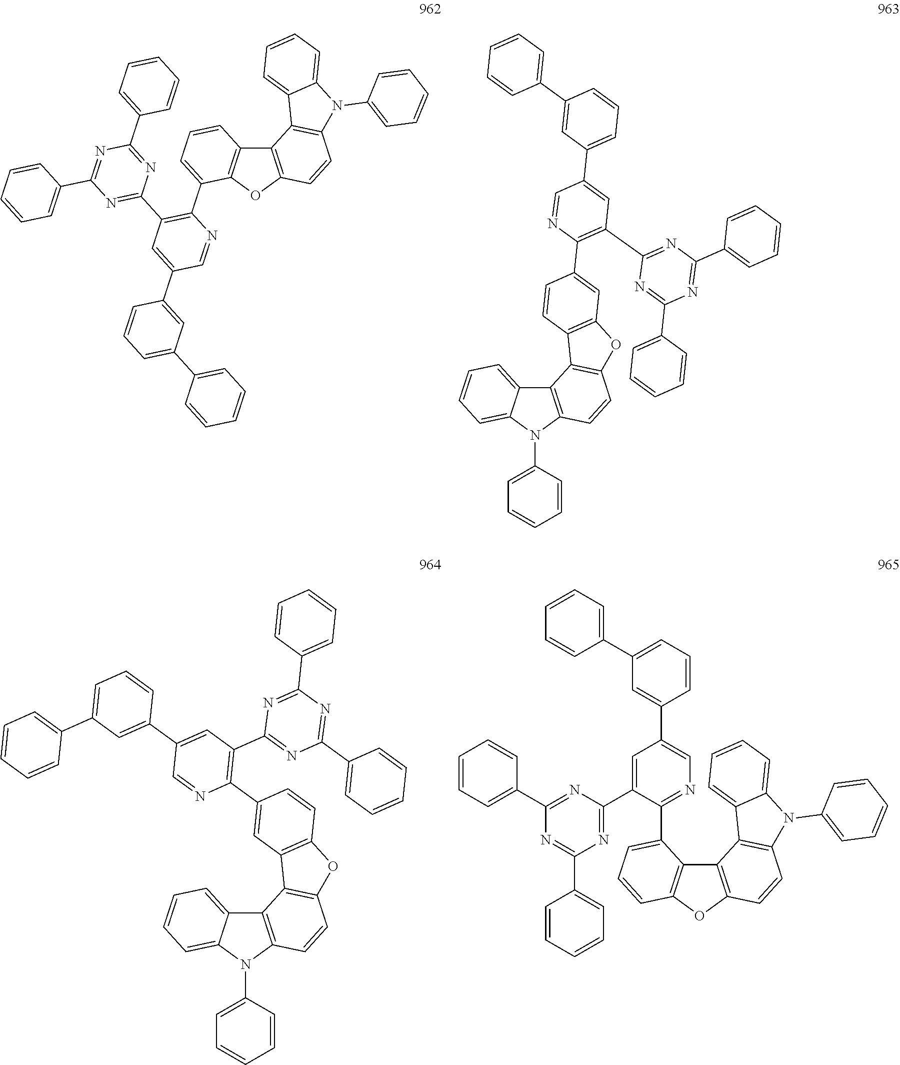 [SMILES: c1ccc(-c2cccc(-c3cnc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3cnc(-c4cccc5oc6ccc7c(c8ccccc8n7-c7ccccc7)c6c45)c(-c4nc(-c5ccccc5)nc(-c5ccccc5)n4)c3)c2)cc1]

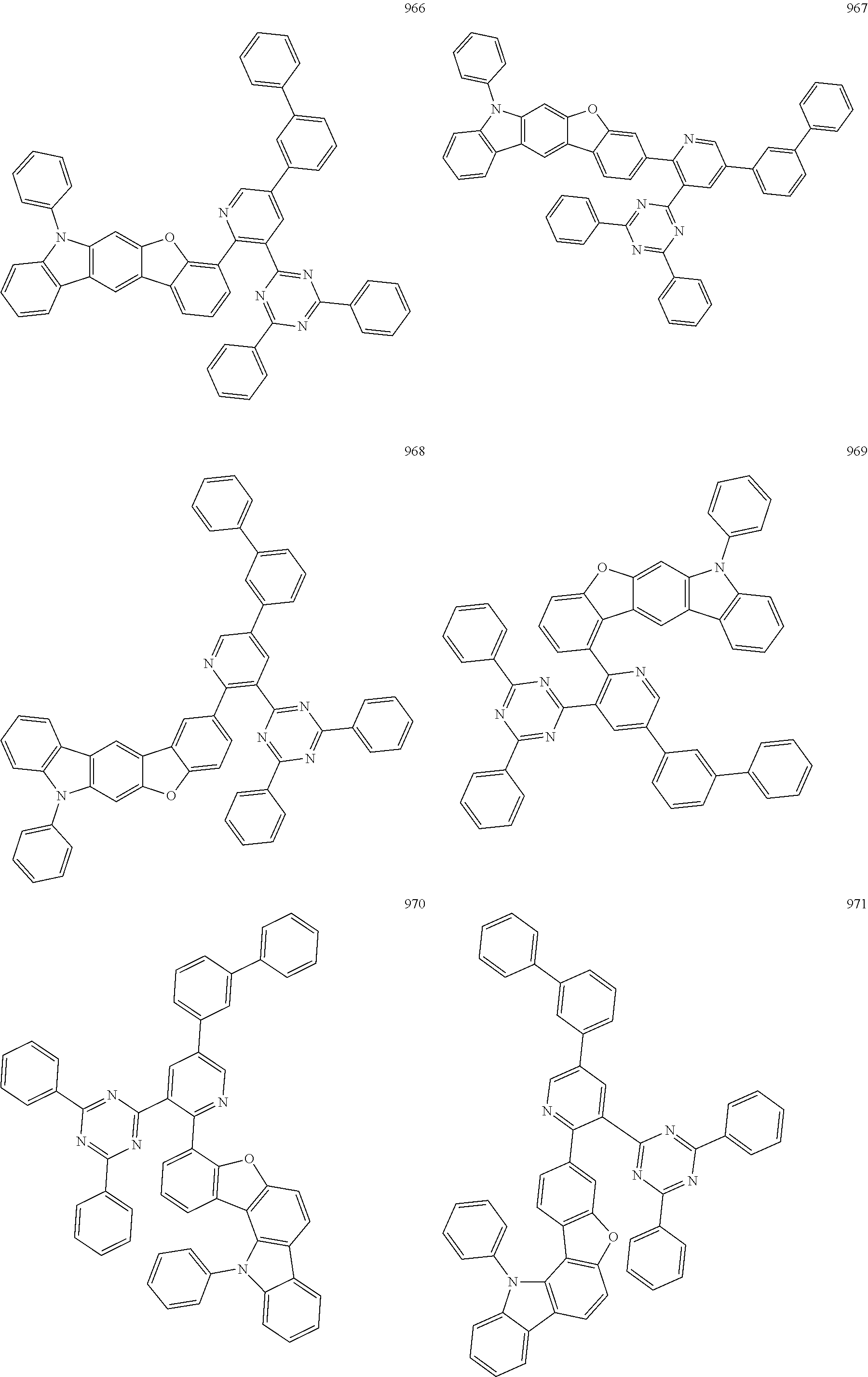

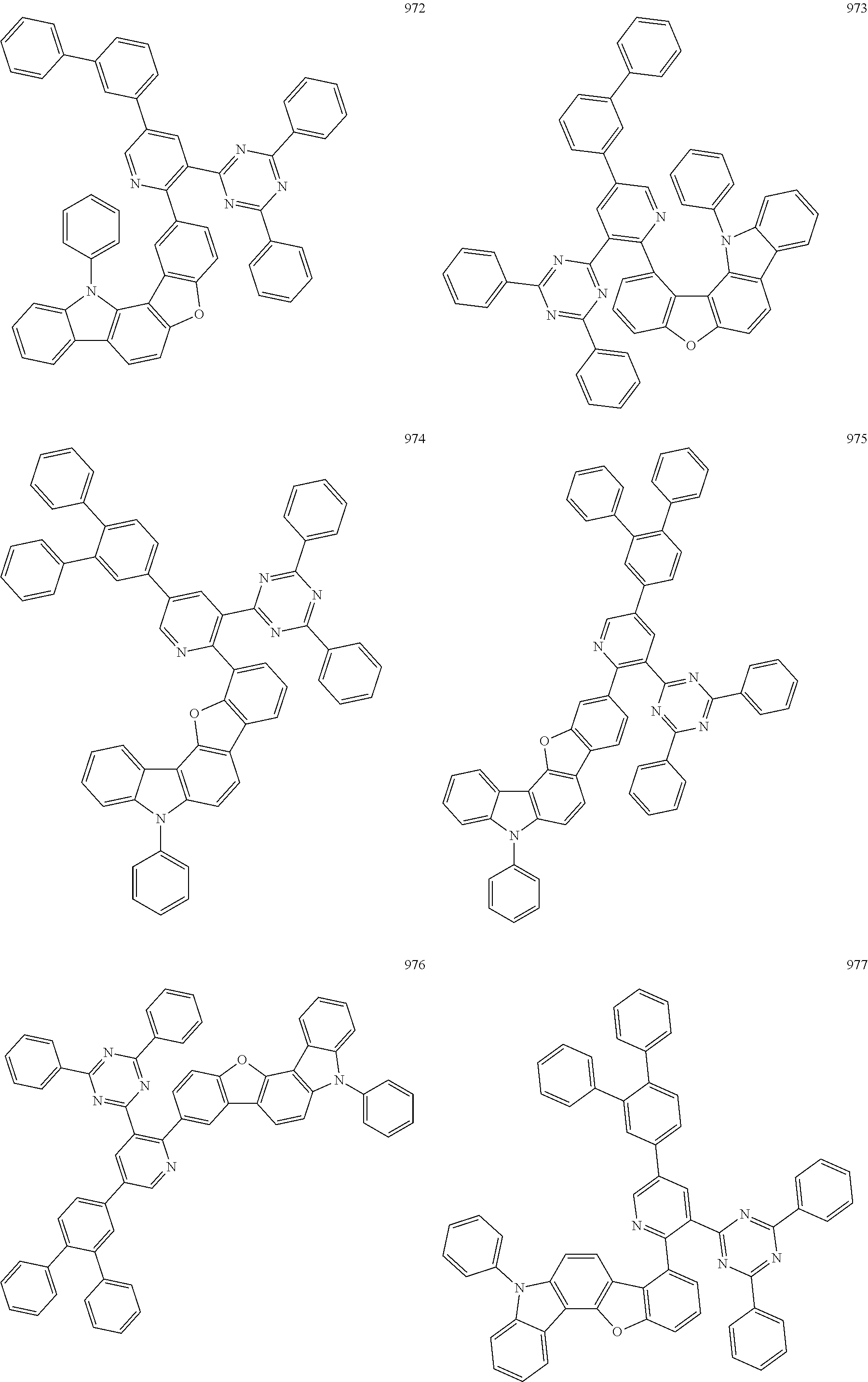

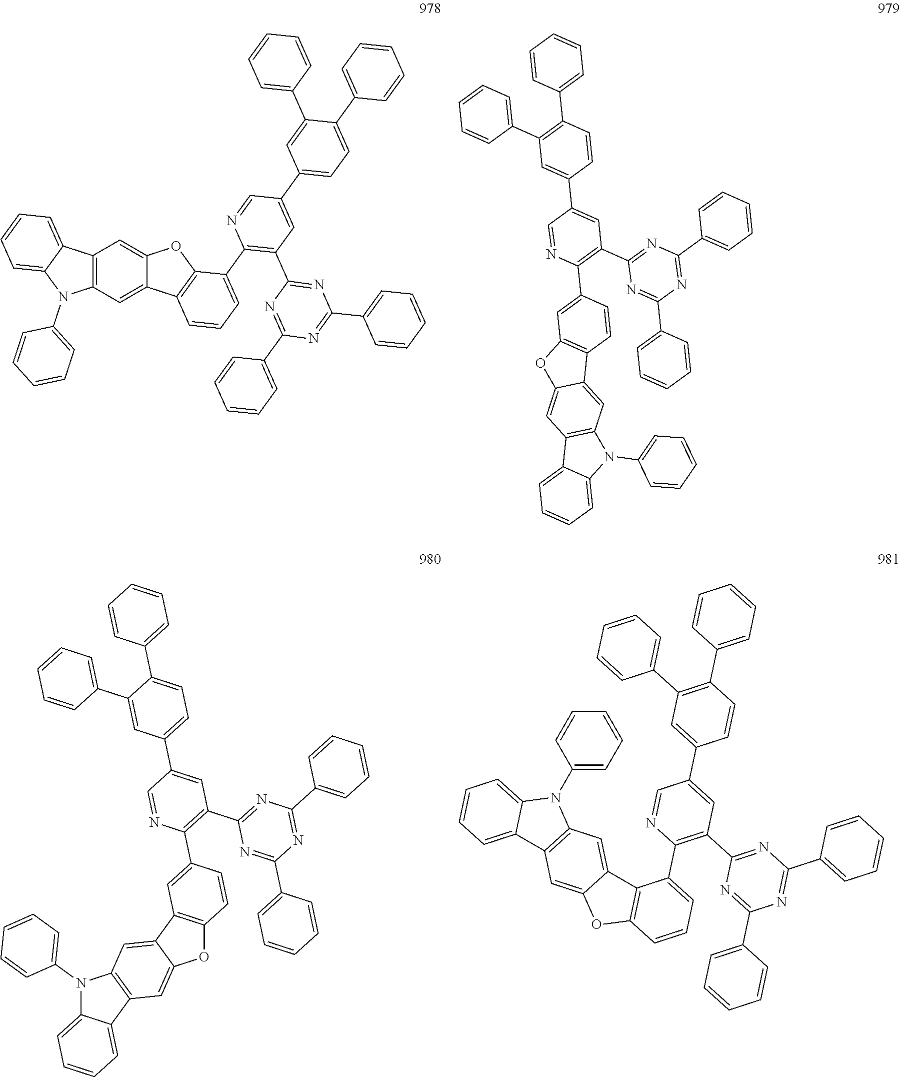 [SMILES: c1ccc(-c2nc(-c3ccccc3)nc(-c3cc(-c4ccc(-c5ccccc5)c(-c5ccccc5)c4)cnc3-c3cccc4c3oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4oc5cc6c7ccccc7n(-c7ccccc7)c6cc5c4c3)n2)cc1 ; c1ccc(-c2nc(-c3ccccc3)nc(-c3cc(-c4ccc(-c5ccccc5)c(-c5ccccc5)c4)cnc3-c3cccc4oc5cc6c7ccccc7n(-c7ccccc7)c6cc5c34)n2)cc1]

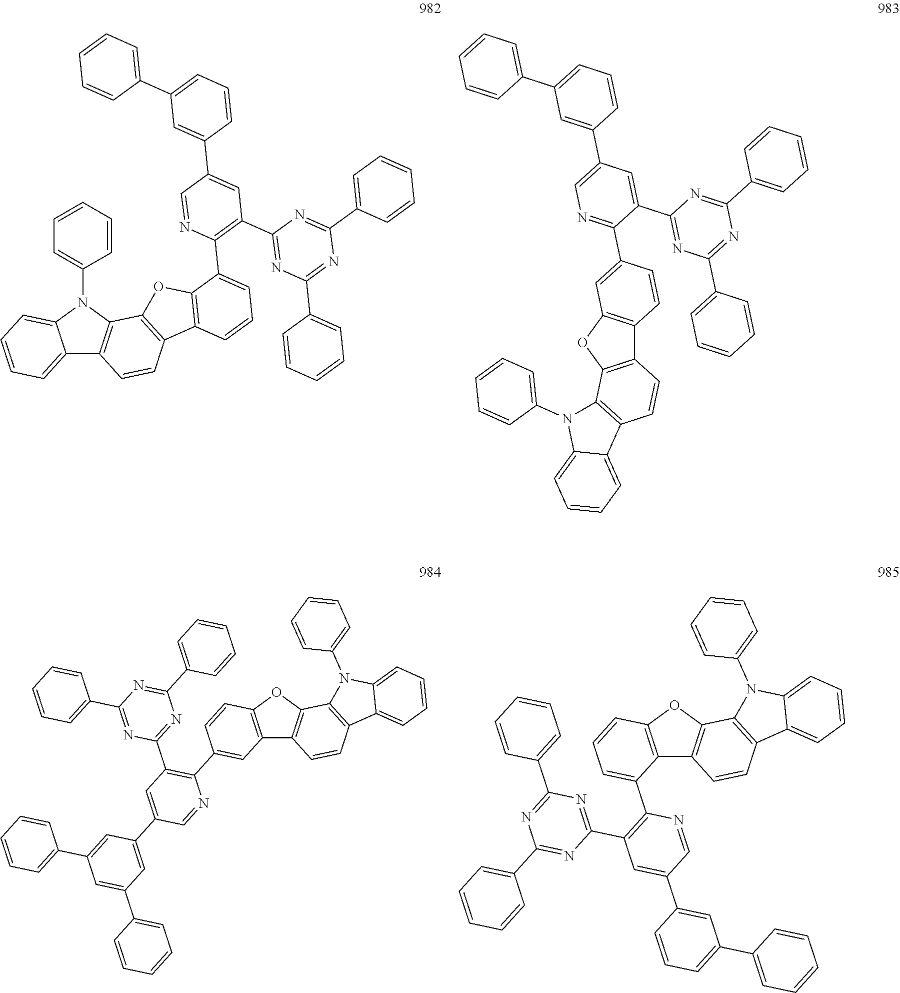 [SMILES: c1ccc(-c2cccc(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

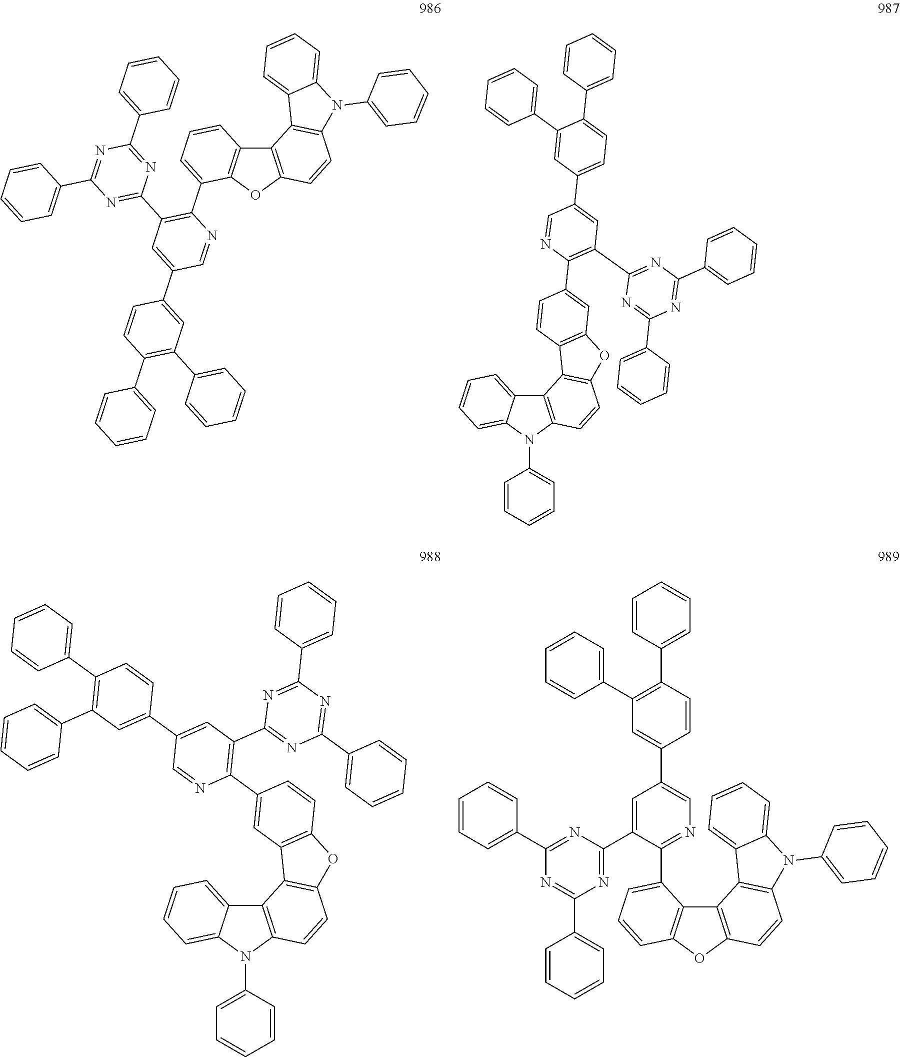 [SMILES: c1ccc(-c2nc(-c3ccccc3)nc(-c3cc(-c4ccc(-c5ccccc5)c(-c5ccccc5)c4)cnc3-c3cccc4c3oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4oc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)cnc3-c3cccc4oc5ccc6c(c7ccccc7n6-c6ccccc6)c5c34)n2)cc1]

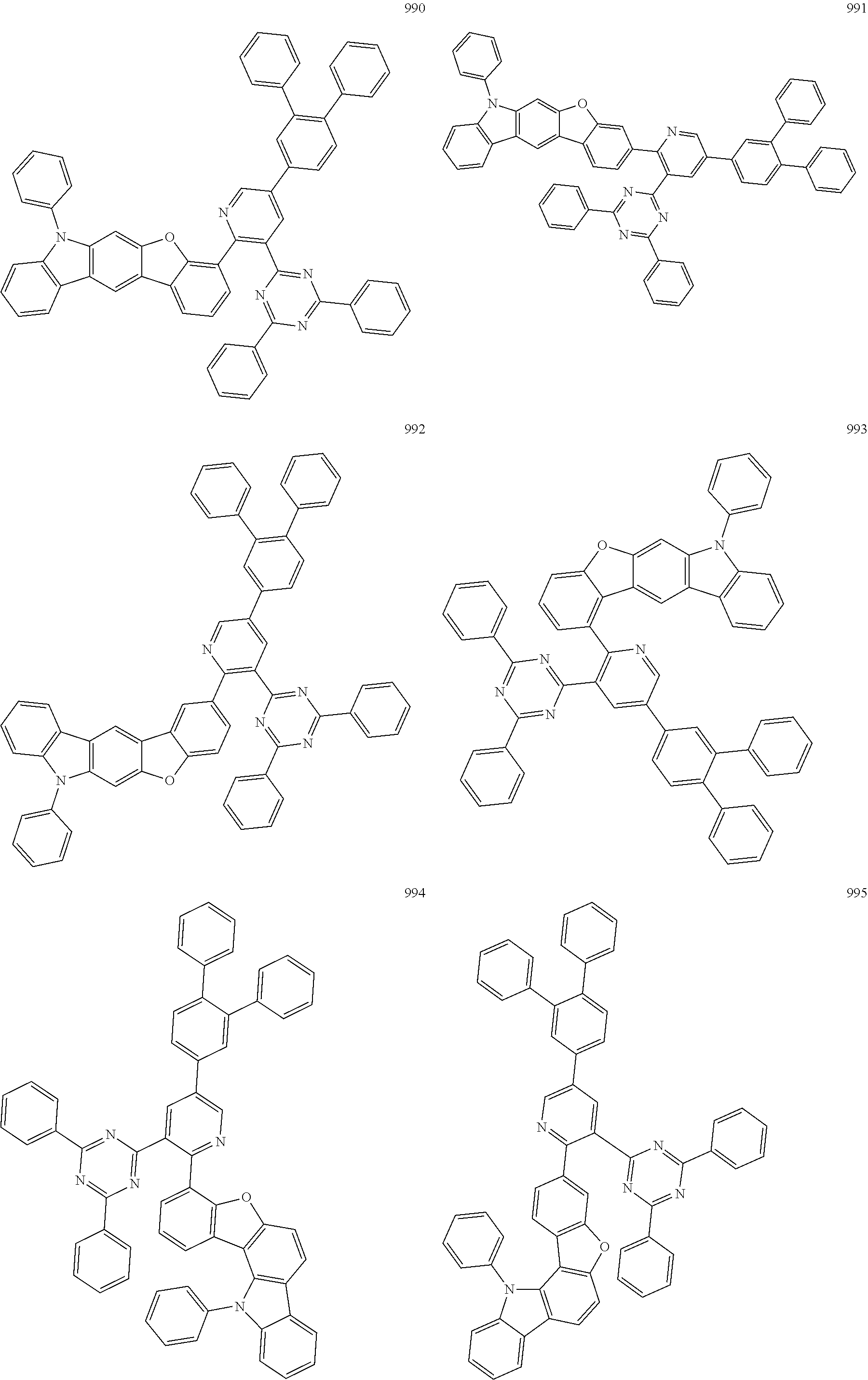

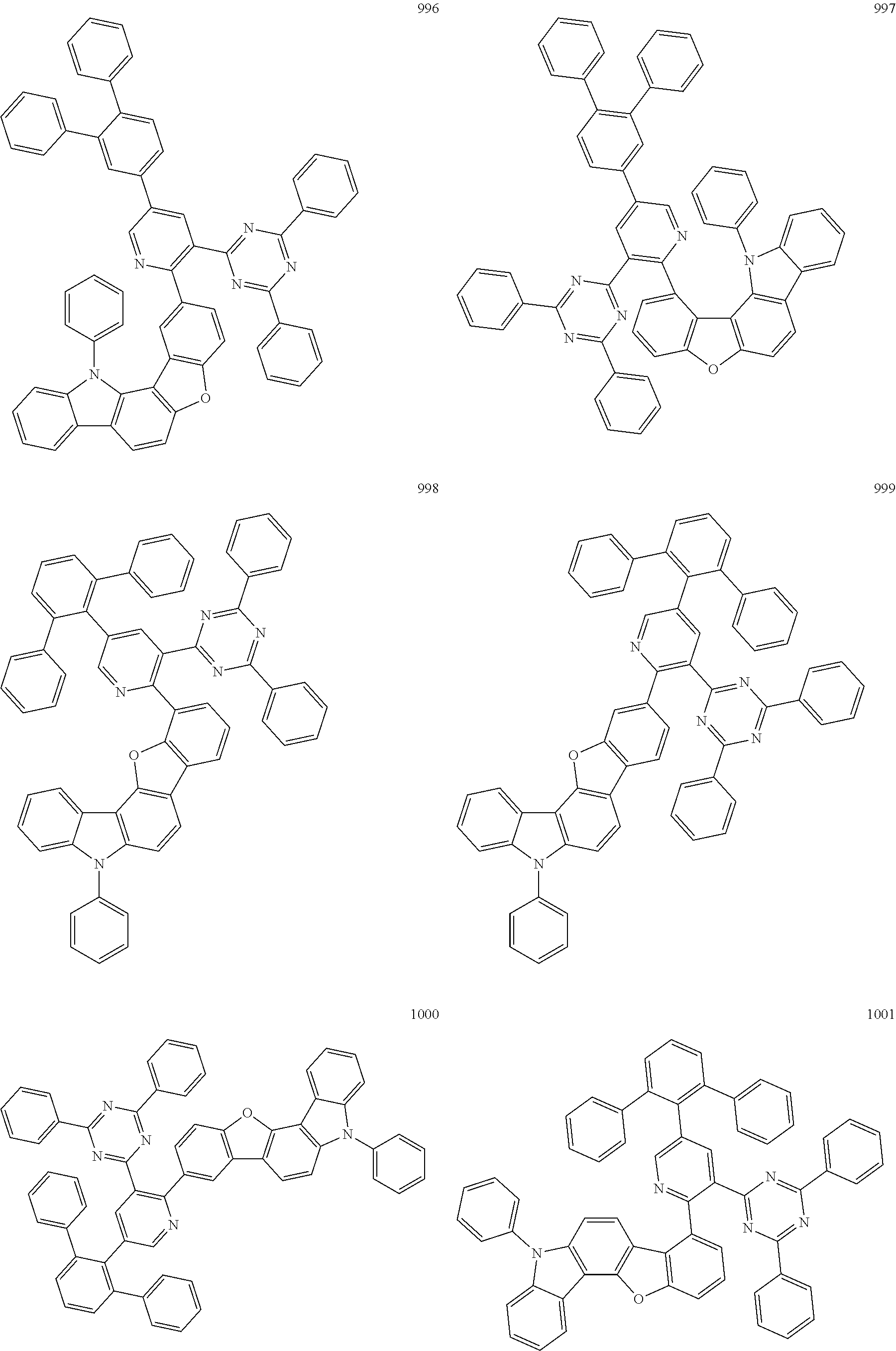

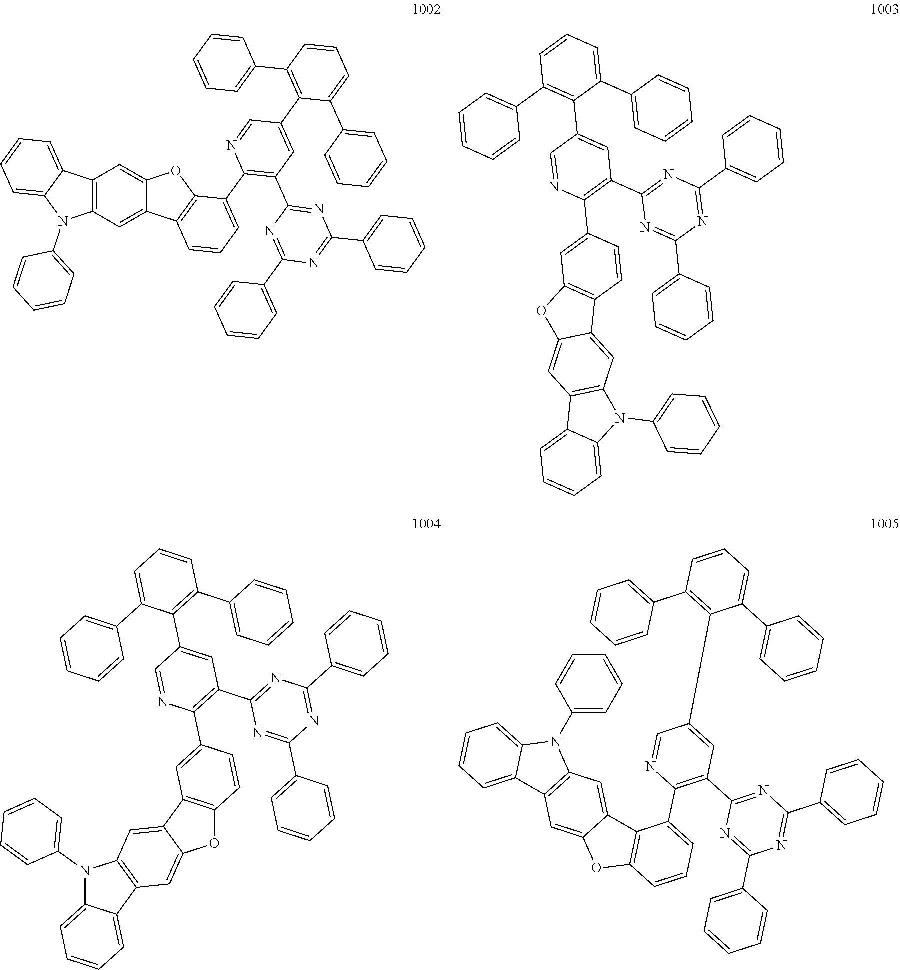 [SMILES: c1ccc(-c2nc(-c3ccccc3)nc(-c3cc(-c4c(-c5ccccc5)cccc4-c4ccccc4)cnc3-c3cccc4c3oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4oc5cc6c7ccccc7n(-c7ccccc7)c6cc5c34)n2)cc1]

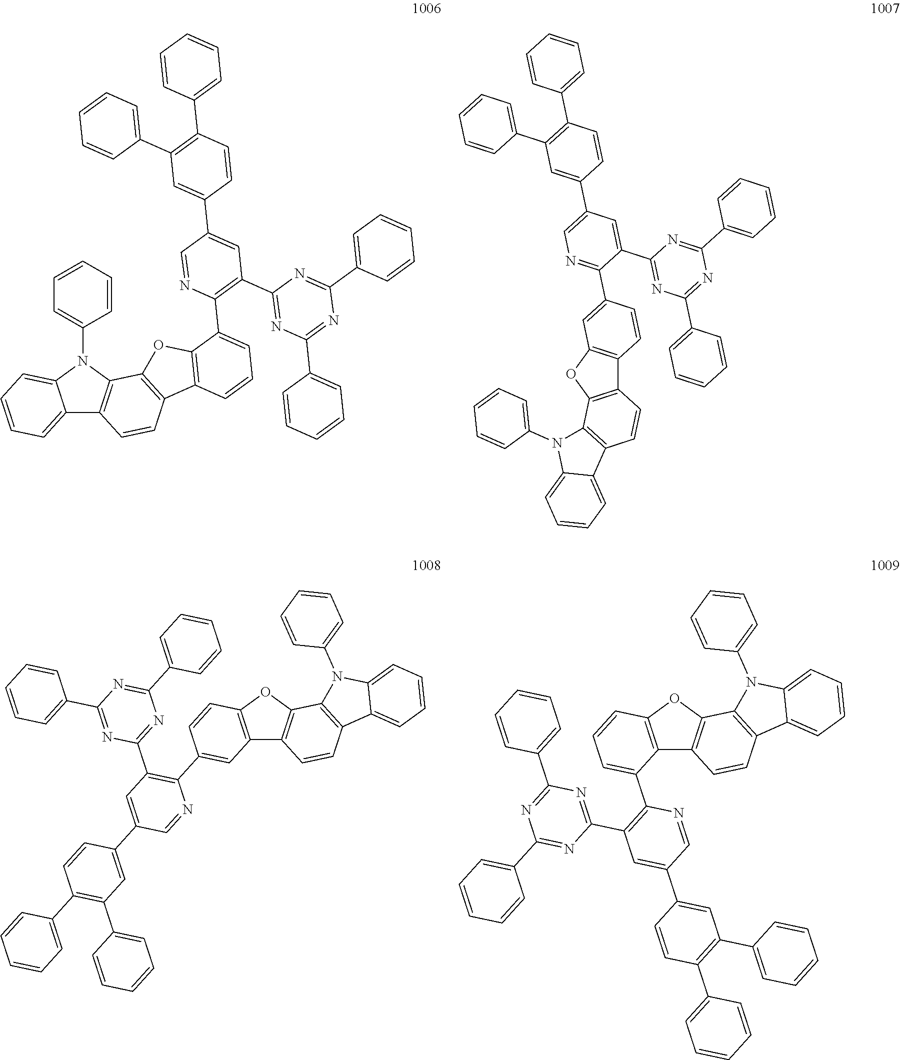 [SMILES: c1ccc(-c2nc(-c3ccccc3)nc(-c3cc(-c4ccc(-c5ccccc5)c(-c5ccccc5)c4)cnc3-c3cccc4c3oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c7ccccc7n(-c7ccccc7)c65)c34)n2)cc1]

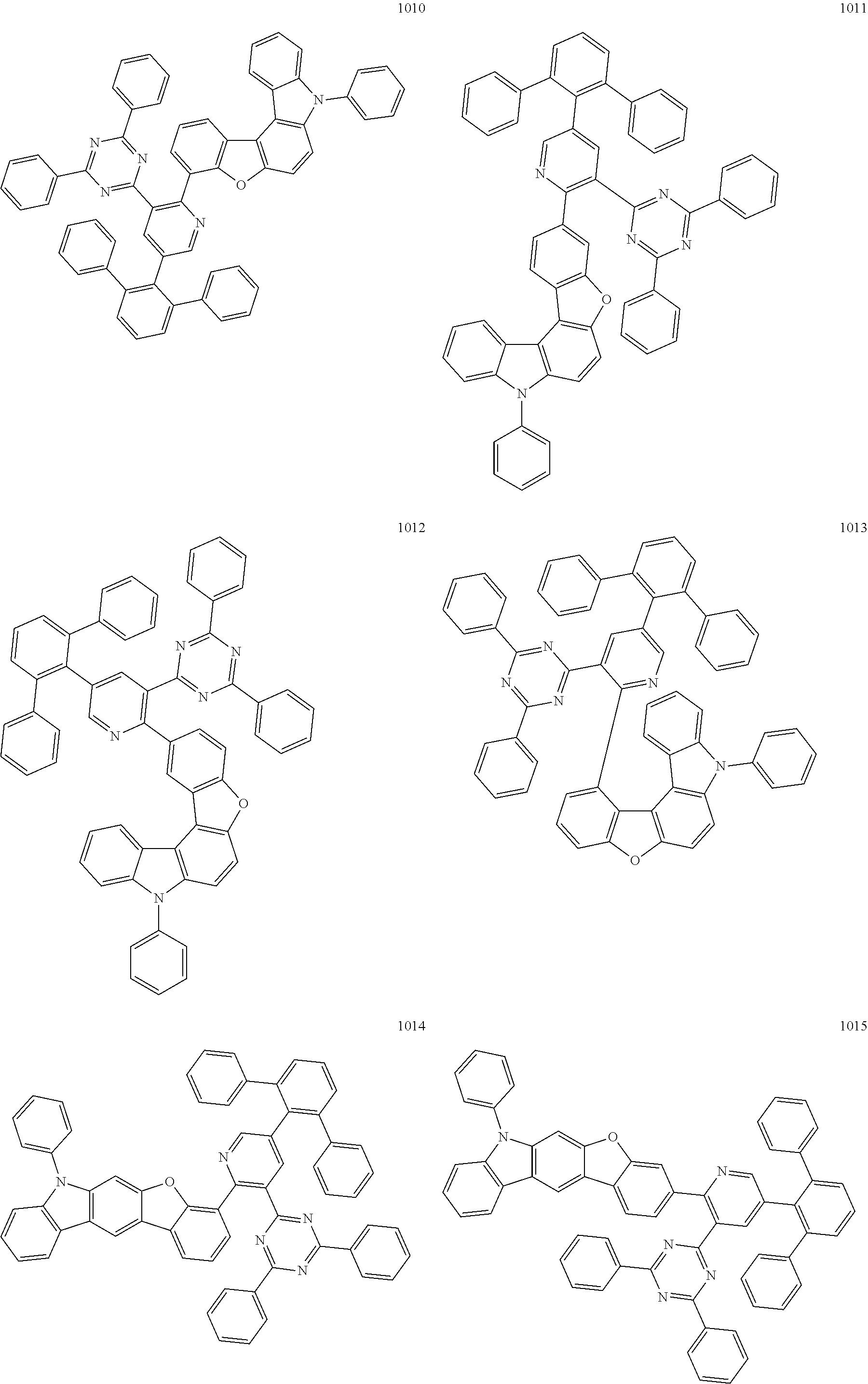

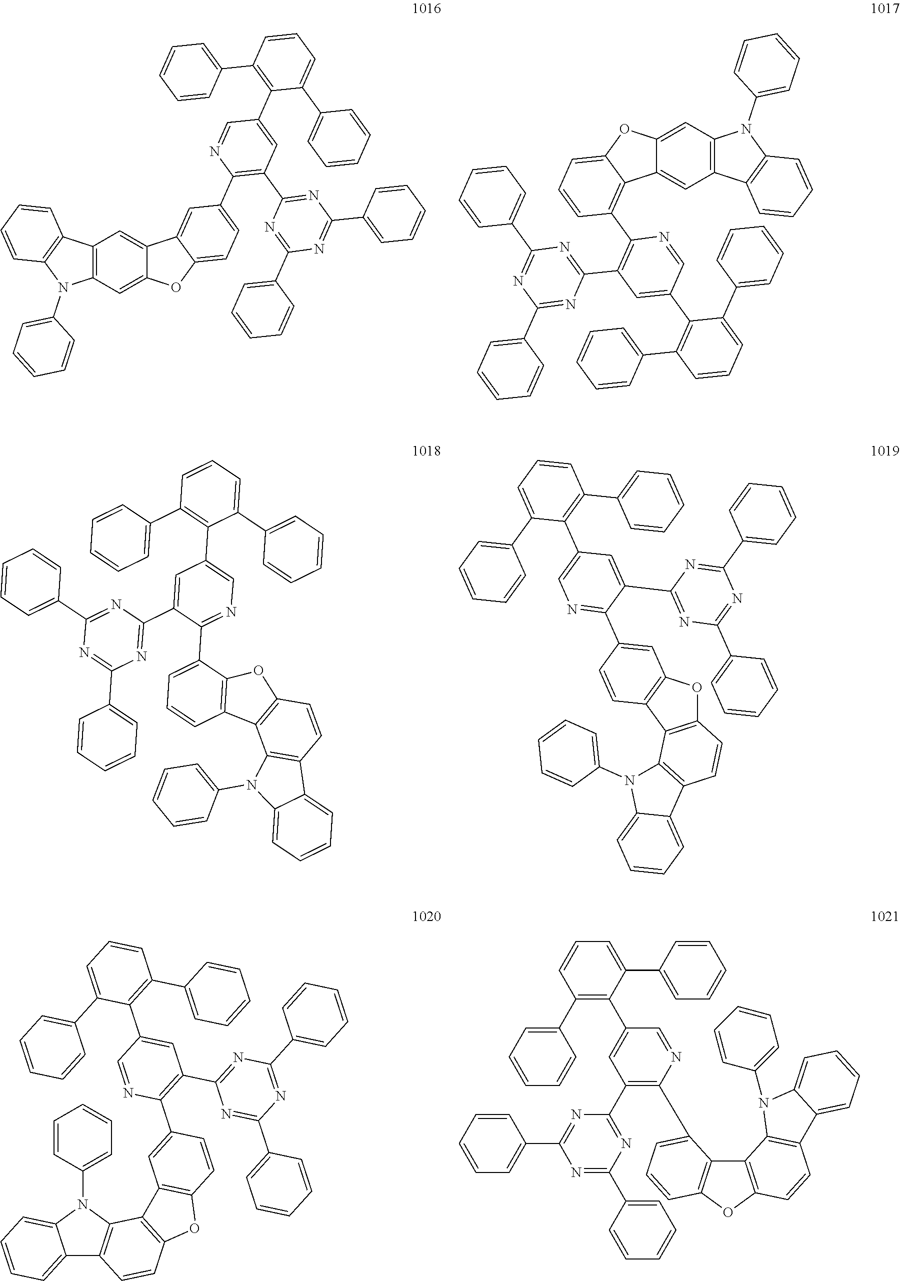

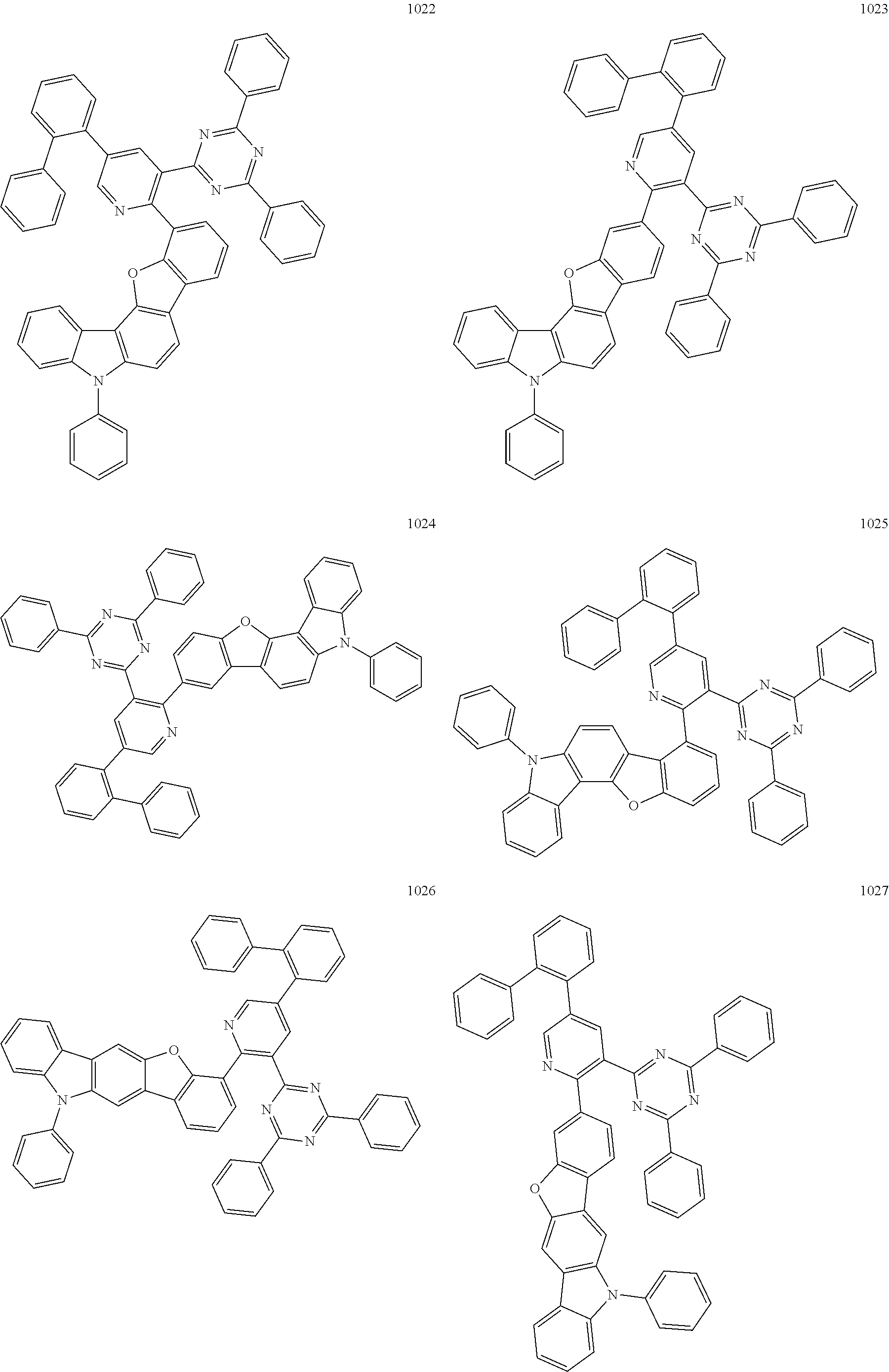

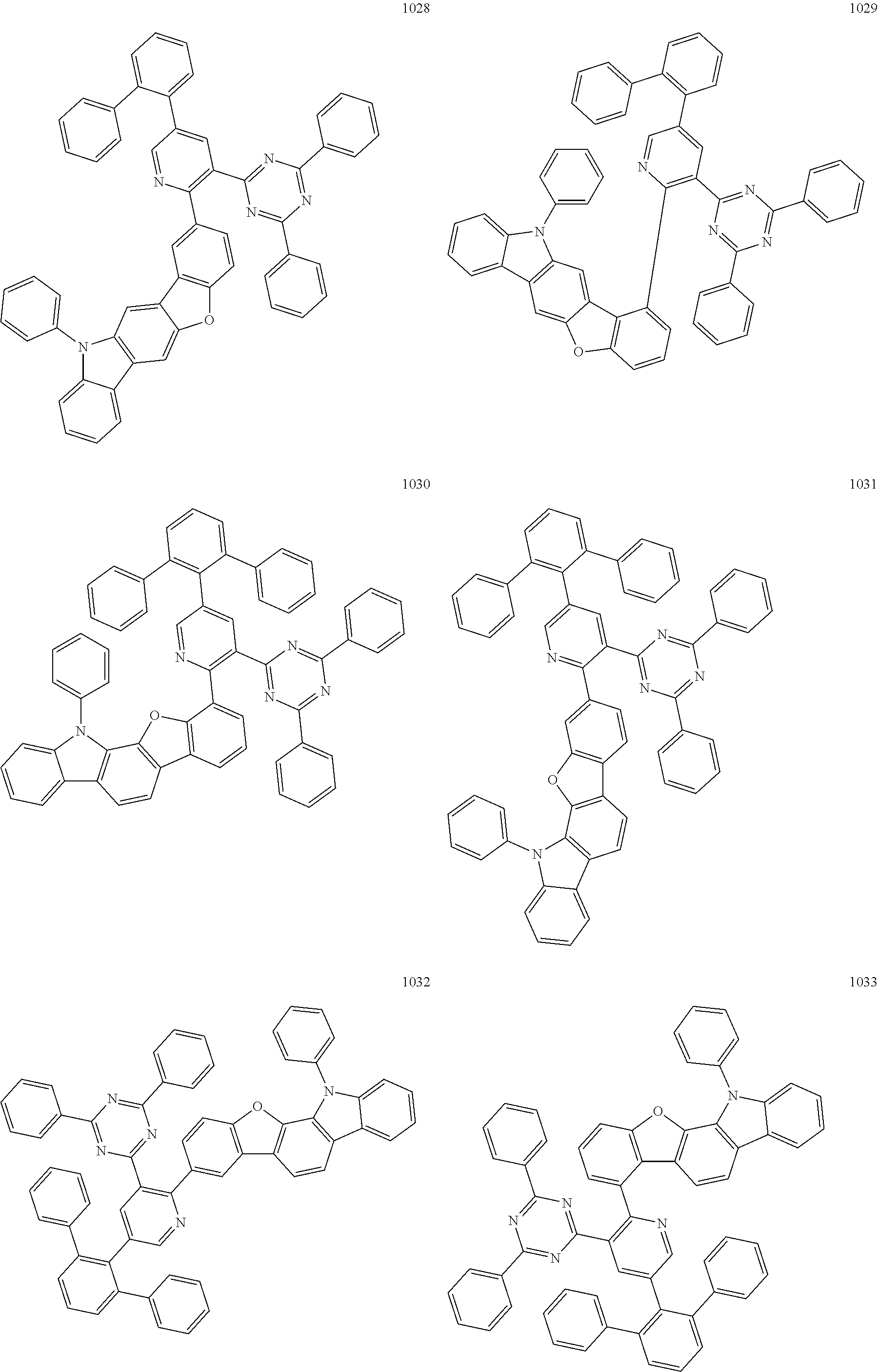

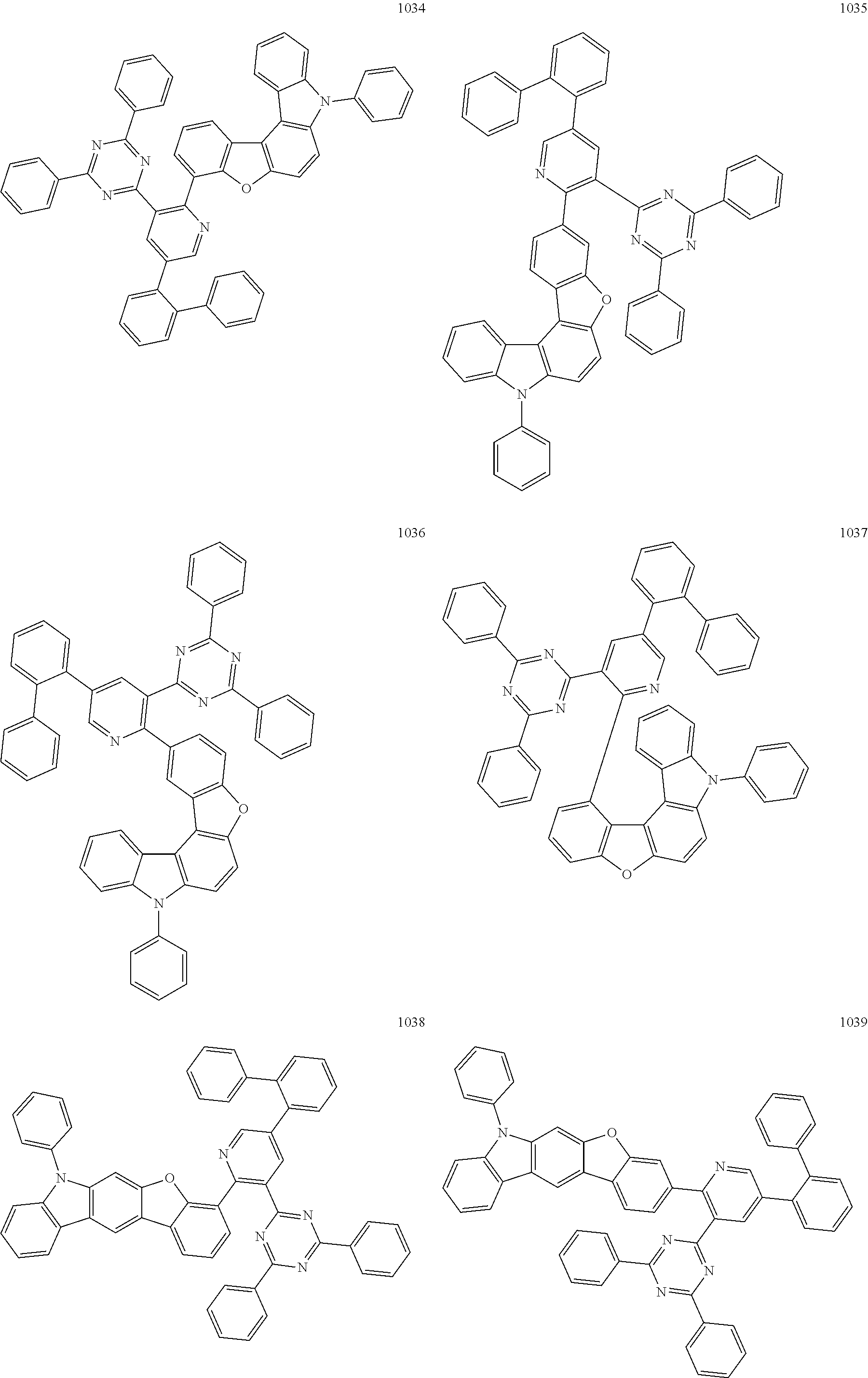

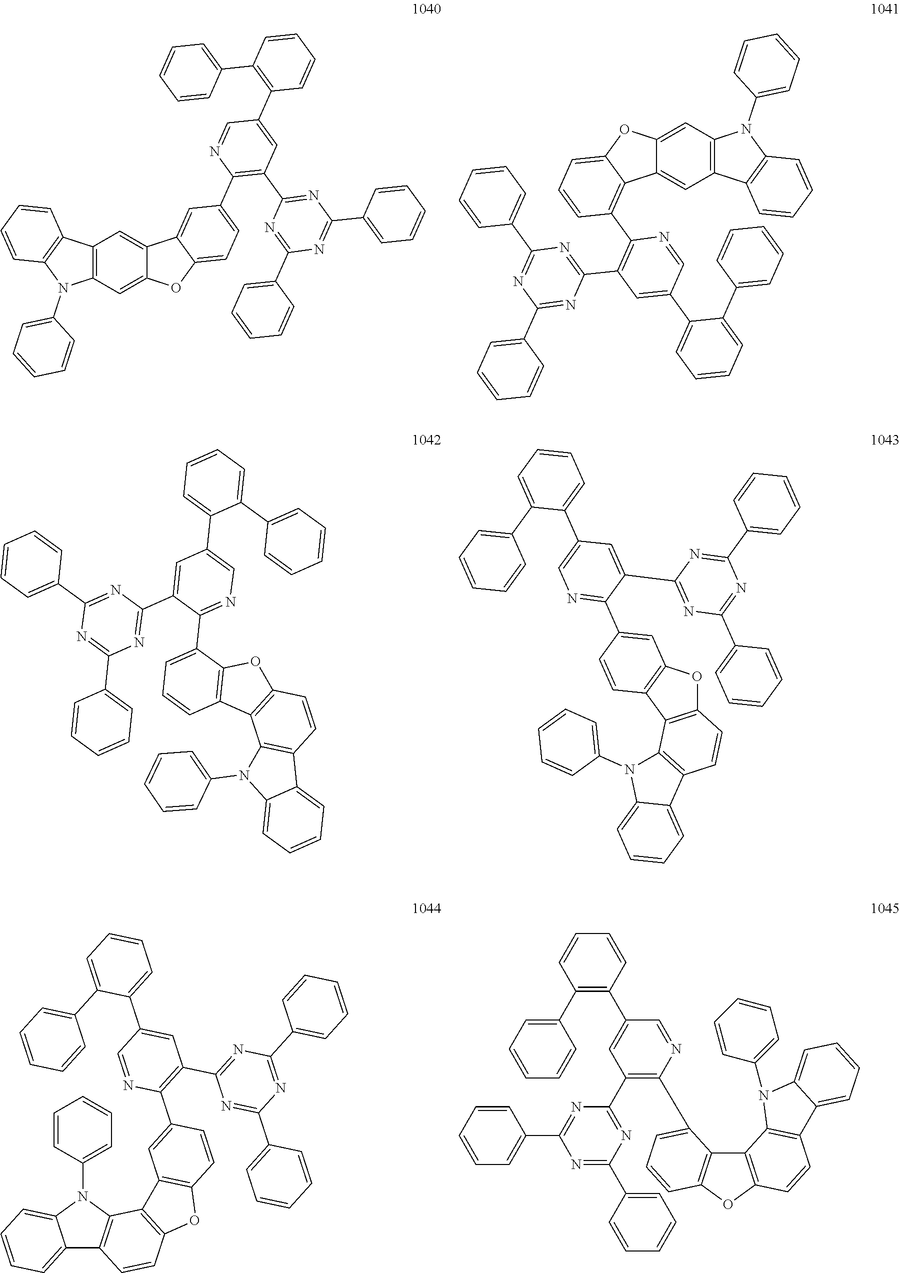

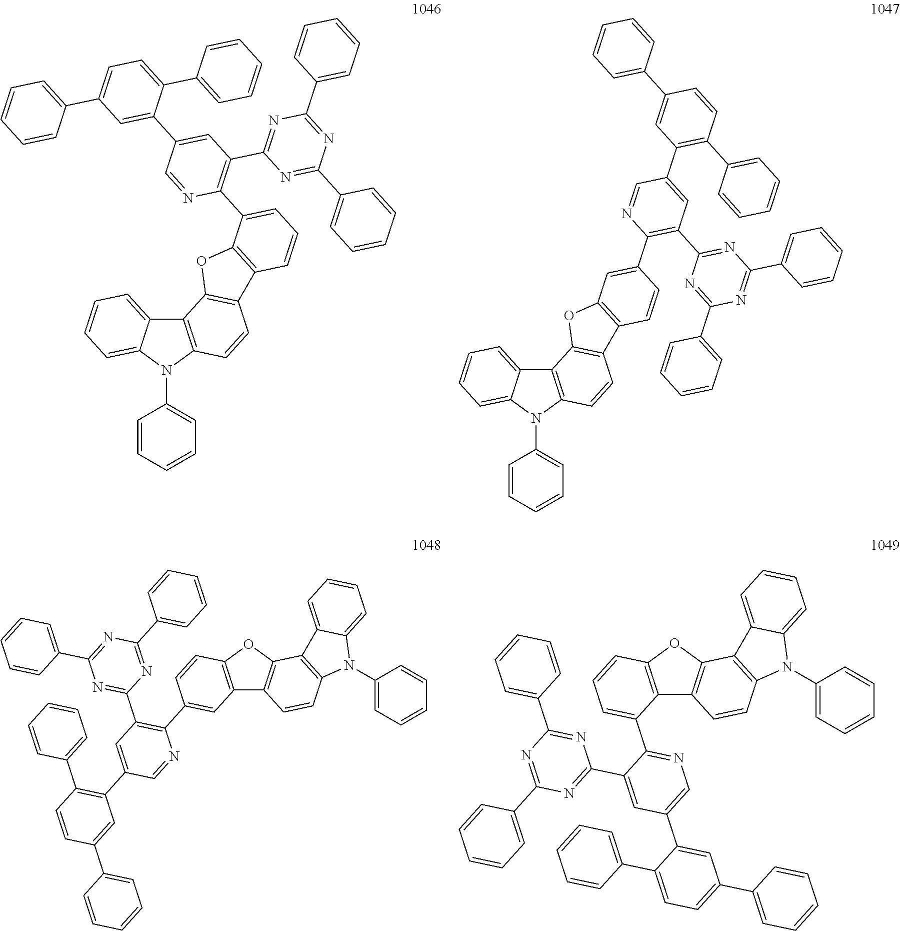 [SMILES: c1ccc(-c2ccc(-c3ccccc3)c(-c3cnc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c6c6ccccc6n7-c6ccccc6)c45)c(-c4nc(-c5ccccc5)nc(-c5ccccc5)n4)c3)c2)cc1]

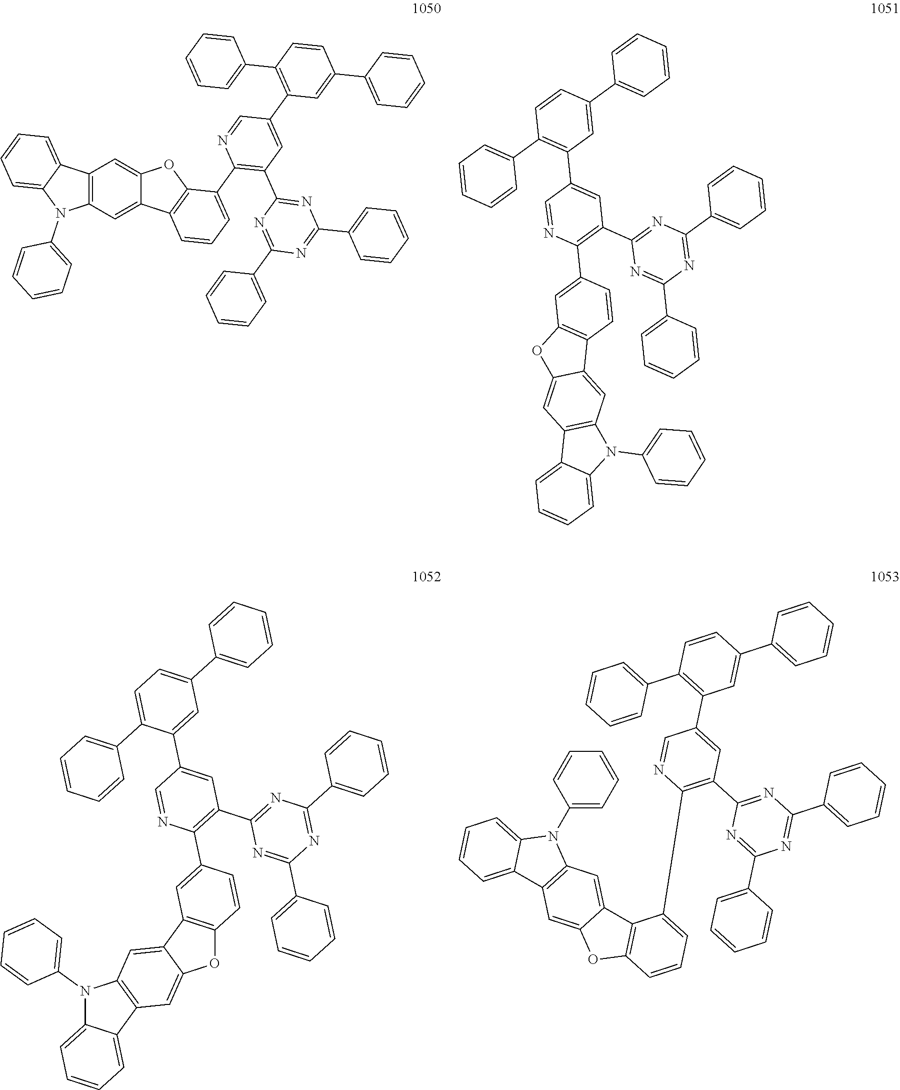 [SMILES: c1ccc(-c2ccc(-c3ccccc3)c(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

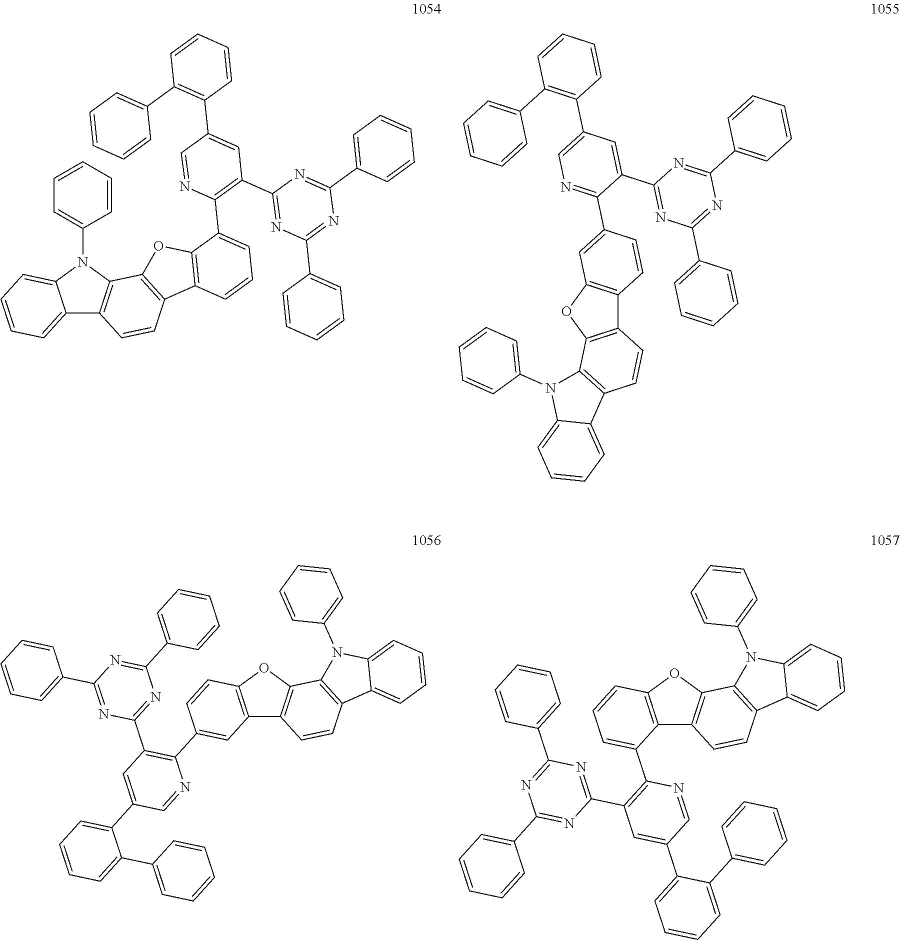 [SMILES: c1ccc(-c2nc(-c3ccccc3)nc(-c3cc(-c4ccccc4-c4ccccc4)cnc3-c3cccc4c3oc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)oc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)cnc3-c3cccc4oc5c(ccc6c7ccccc7n(-c7ccccc7)c65)c34)n2)cc1]

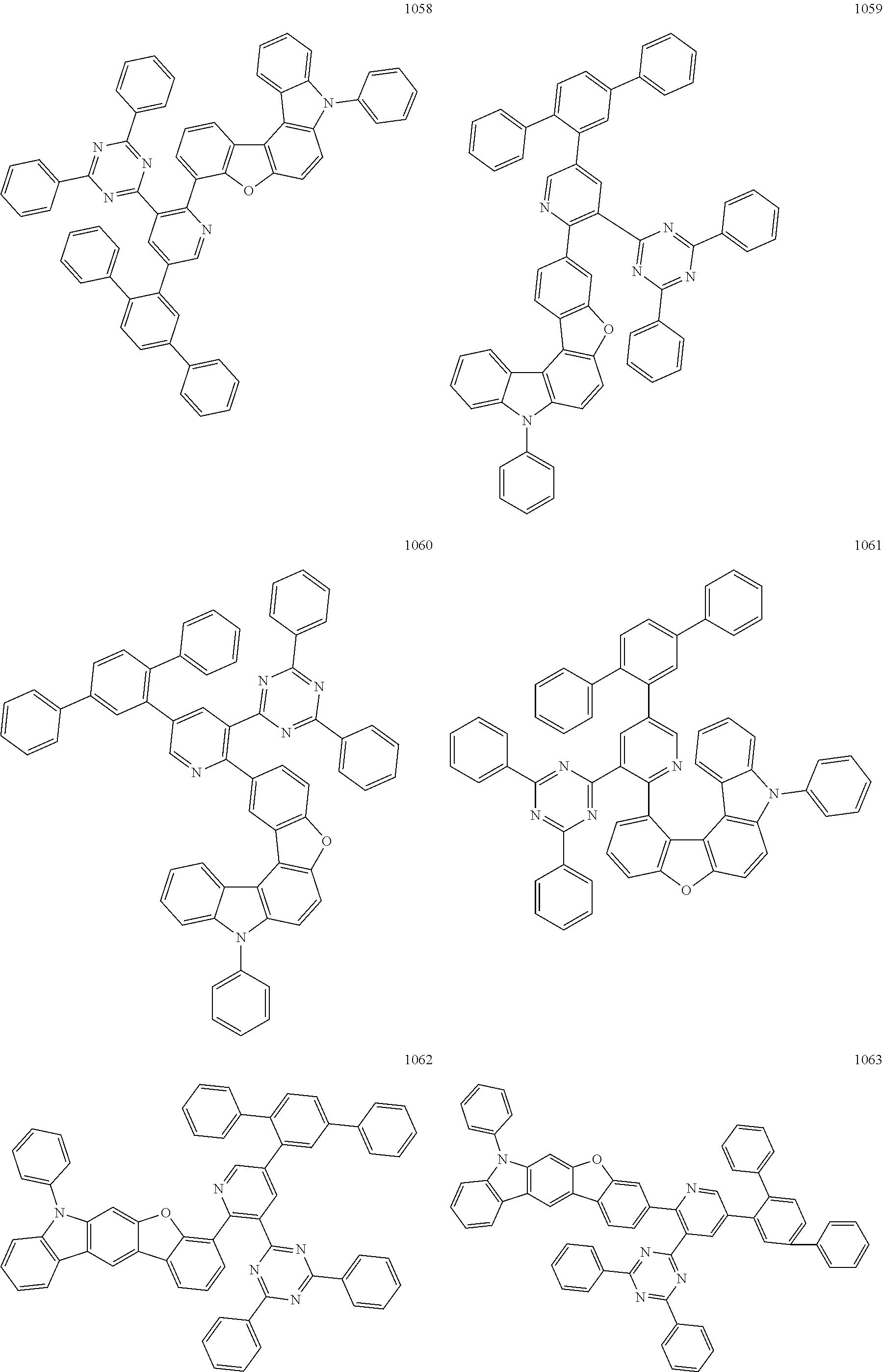

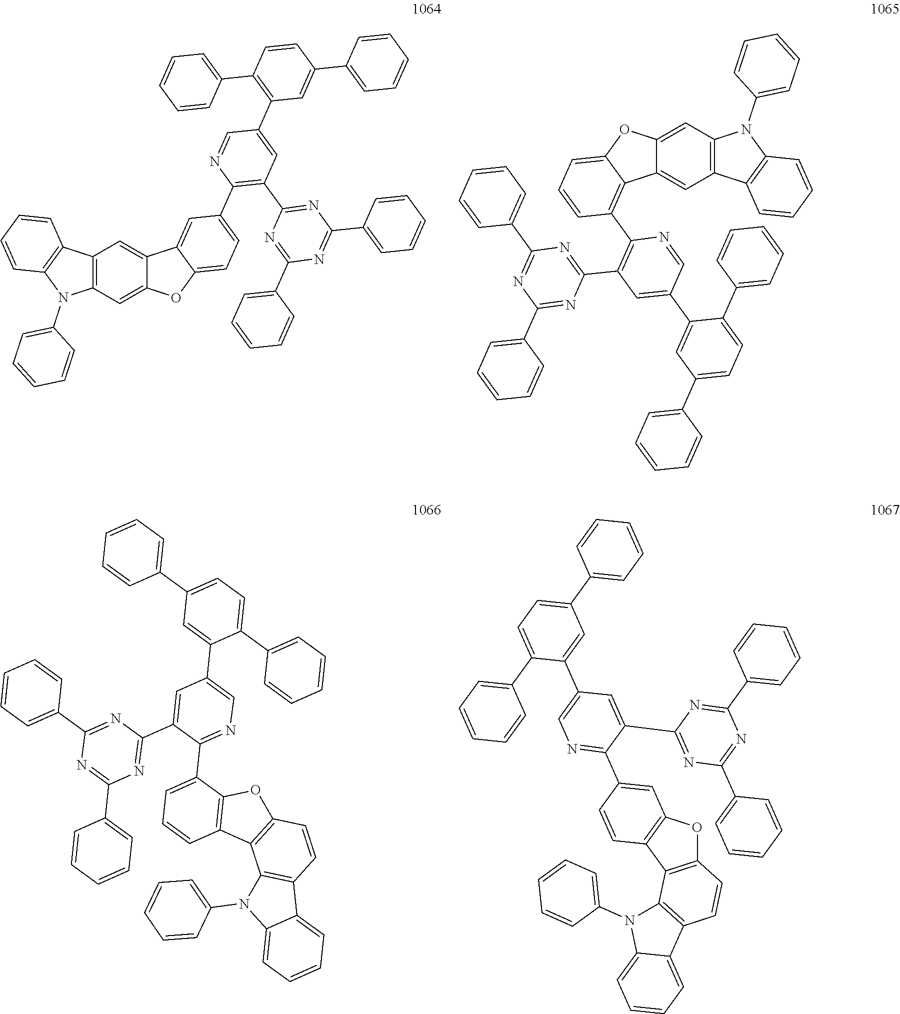

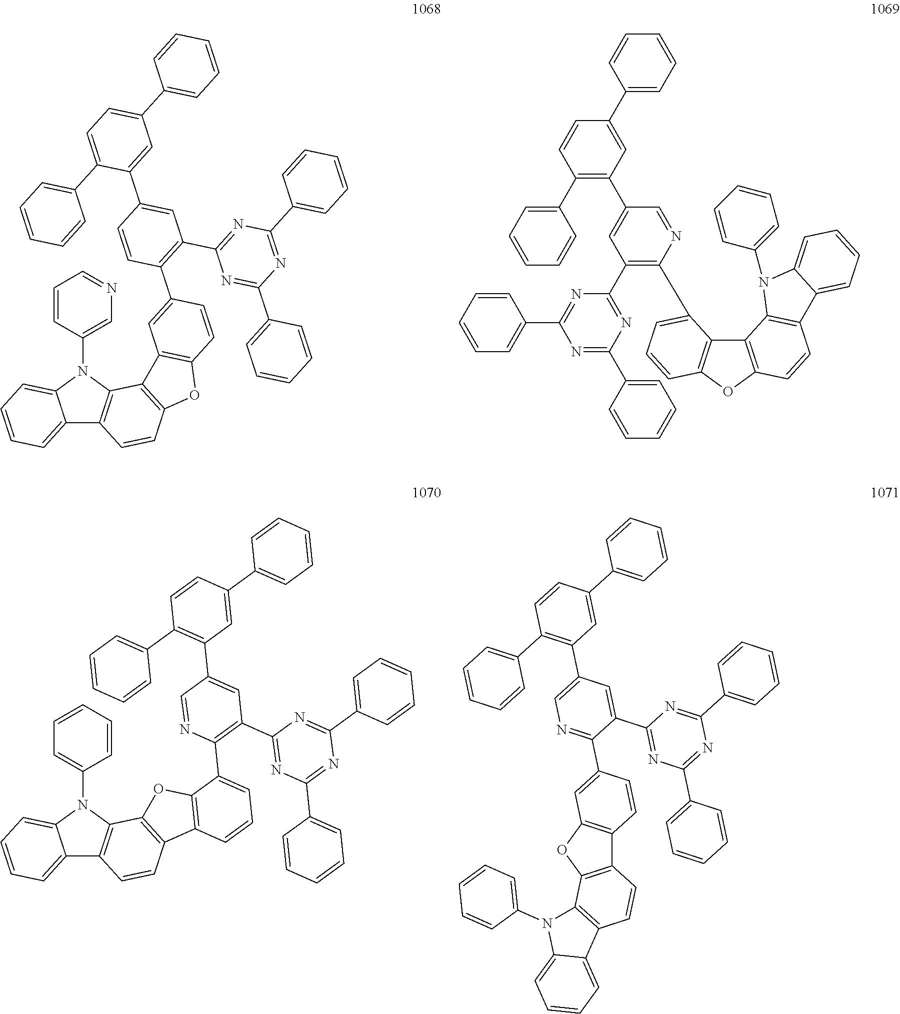 [SMILES: c1ccc(-c2ccc(-c3ccccc3)c(-c3ccc(-c4ccc5oc6ccc7c8ccccc8n(-c8cccnc8)c7c6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1]

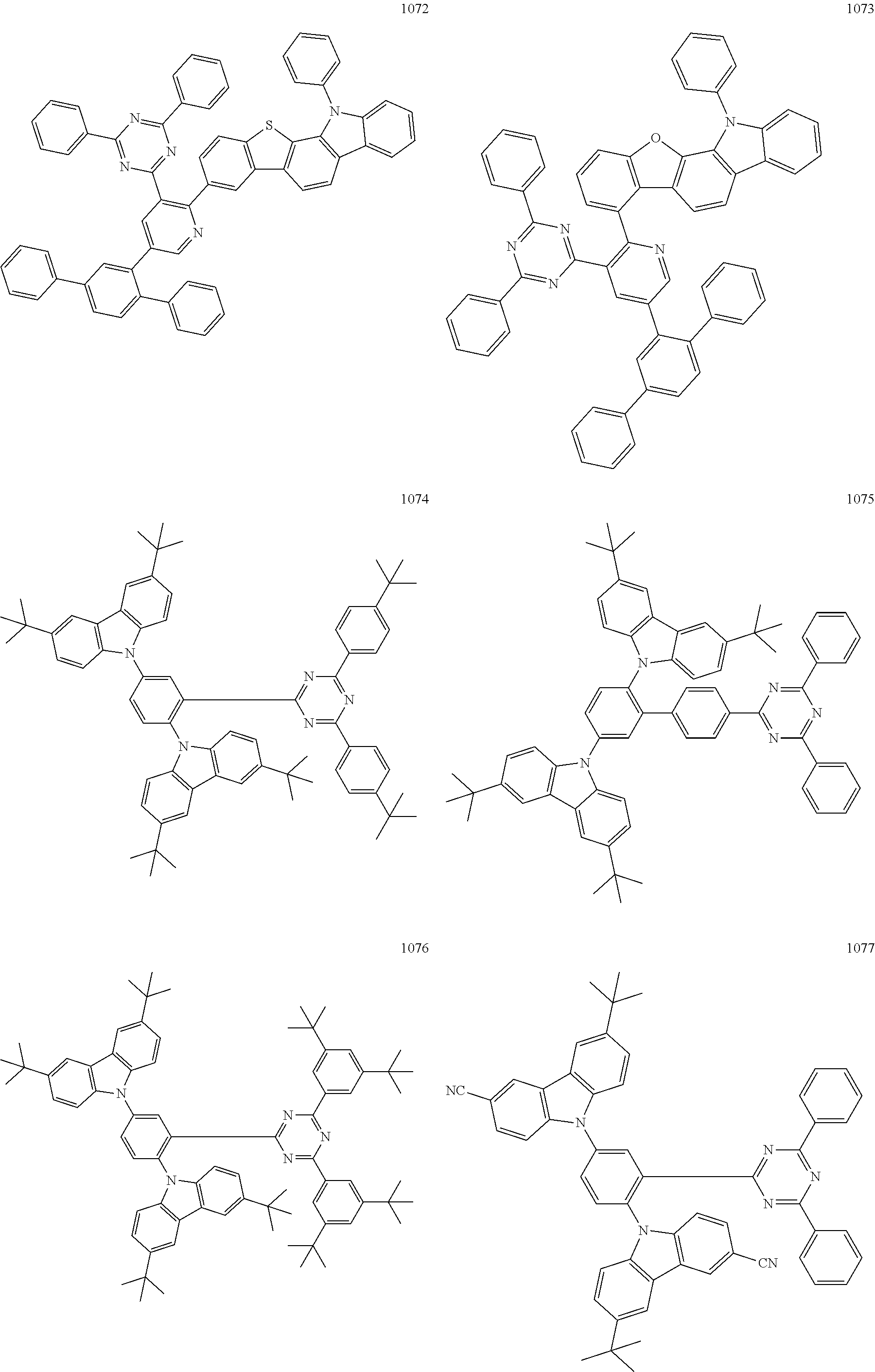

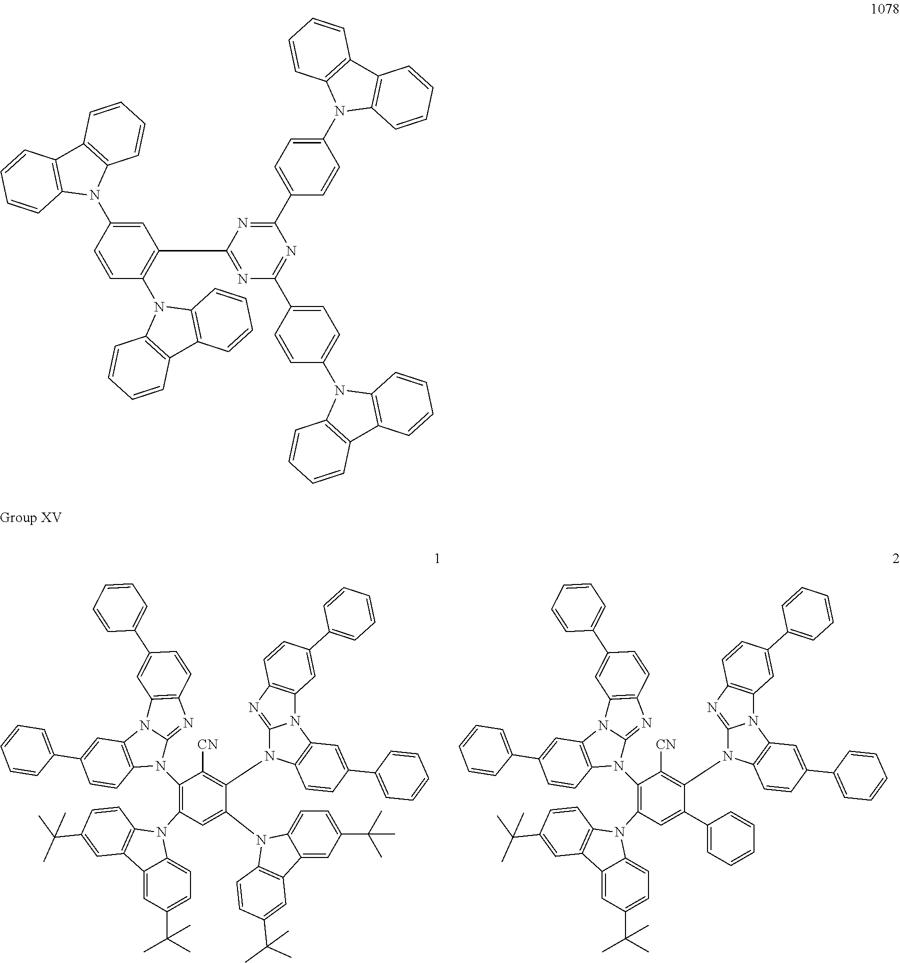 [SMILES: c1ccc2c(c1)c1ccccc1n2-c1ccc(-c2nc(-c3ccc(-n4c5ccccc5c5ccccc54)cc3)nc(-c3cc(-n4c5ccccc5c5ccccc54)ccc3-n3c4ccccc4c4ccccc43)n2)cc1 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-n2c3ccc(-c4ccccc4)cc3n3c4cc(-c5ccccc5)ccc4nc23)c(C#N)c1-n1c2ccc(-c3ccccc3)cc2n2c3cc(-c4ccccc4)ccc3nc12 ; CC(C)(C)c1ccc2c(c1)c1cc(C(C)(C)C)ccc1n2-c1cc(-c2ccccc2)c(-n2c3ccc(-c4ccccc4)cc3n3c4cc(-c5ccccc5)ccc4nc23)c(C#N)c1-n1c2ccc(-c3ccccc3)cc2n2c3cc(-c4ccccc4)ccc3nc12]

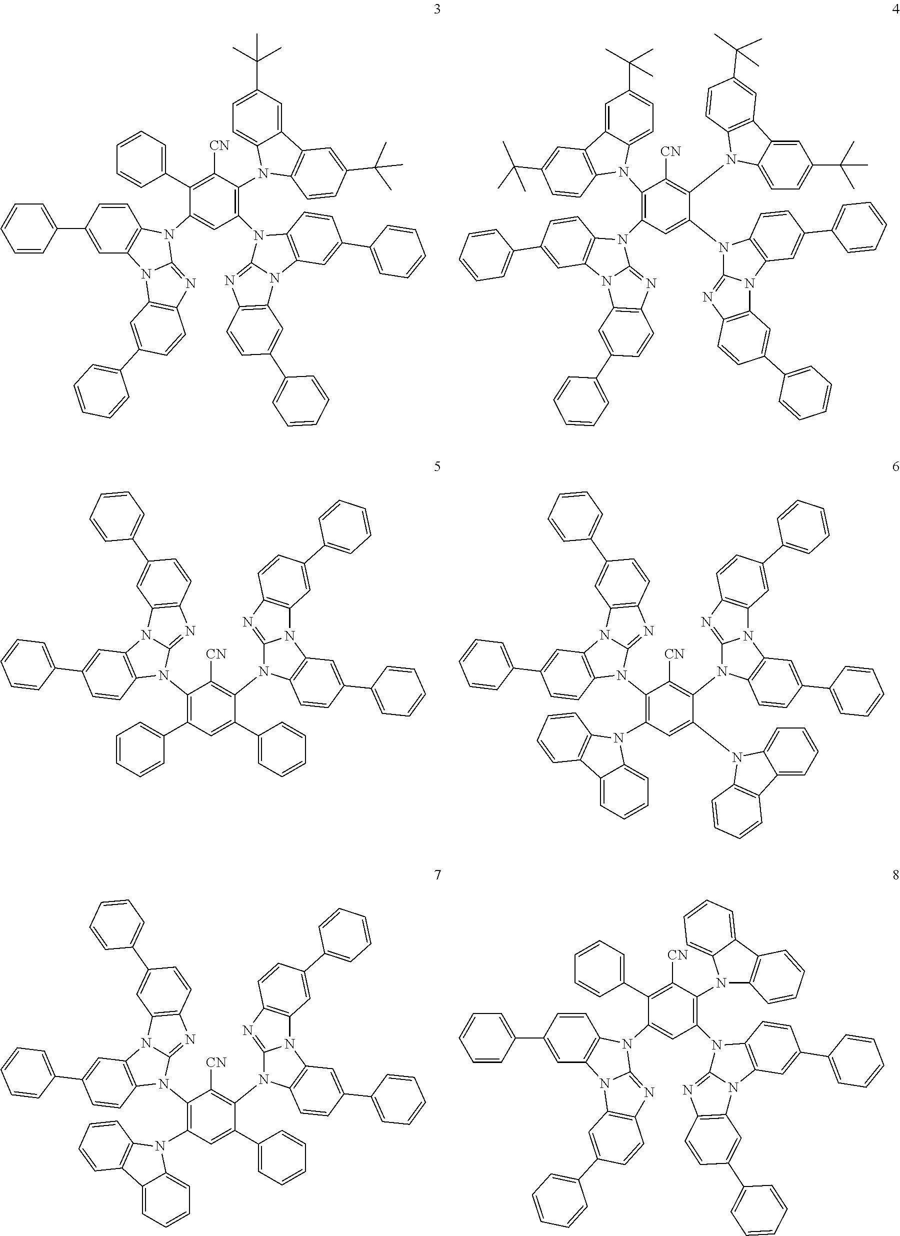

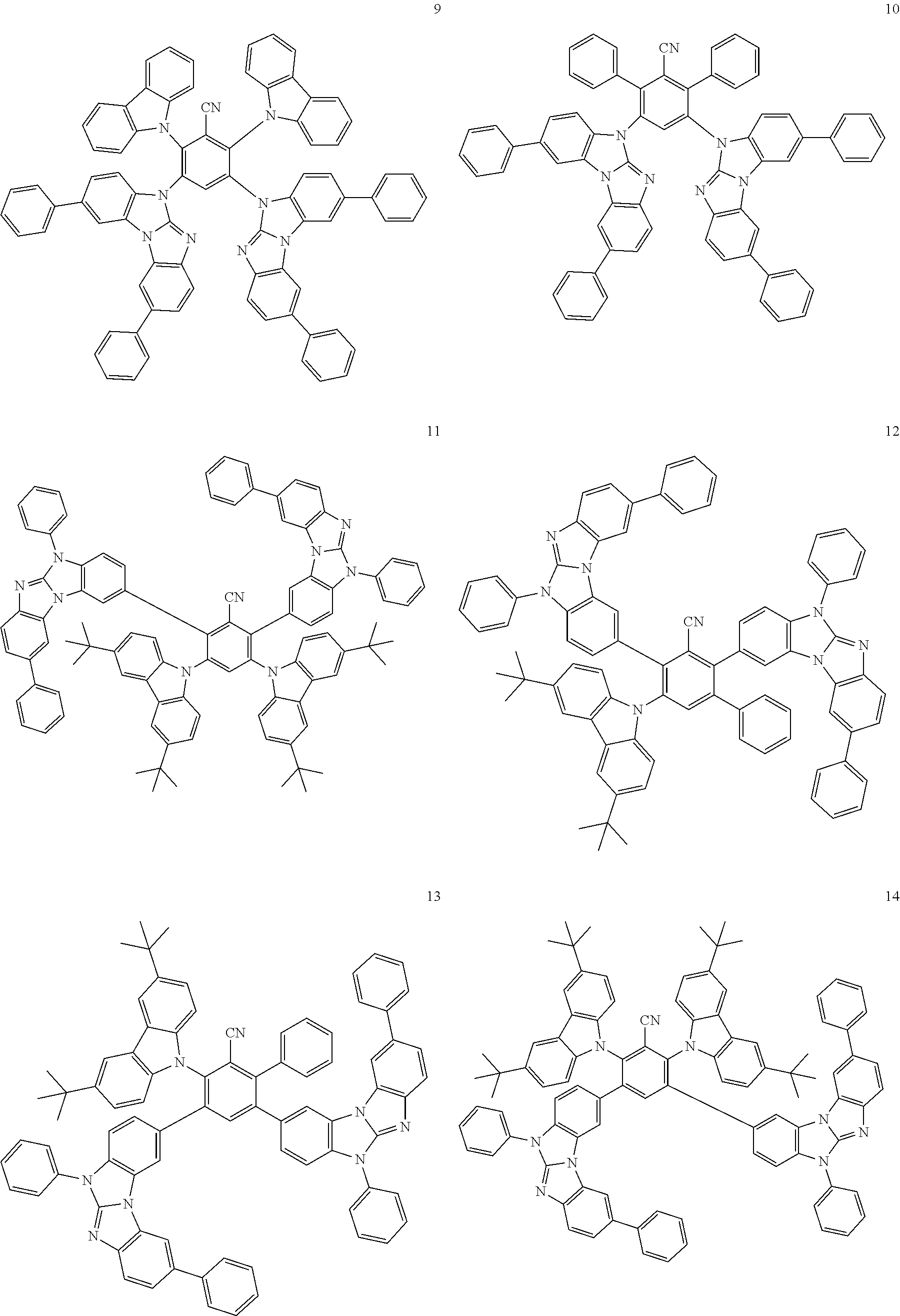

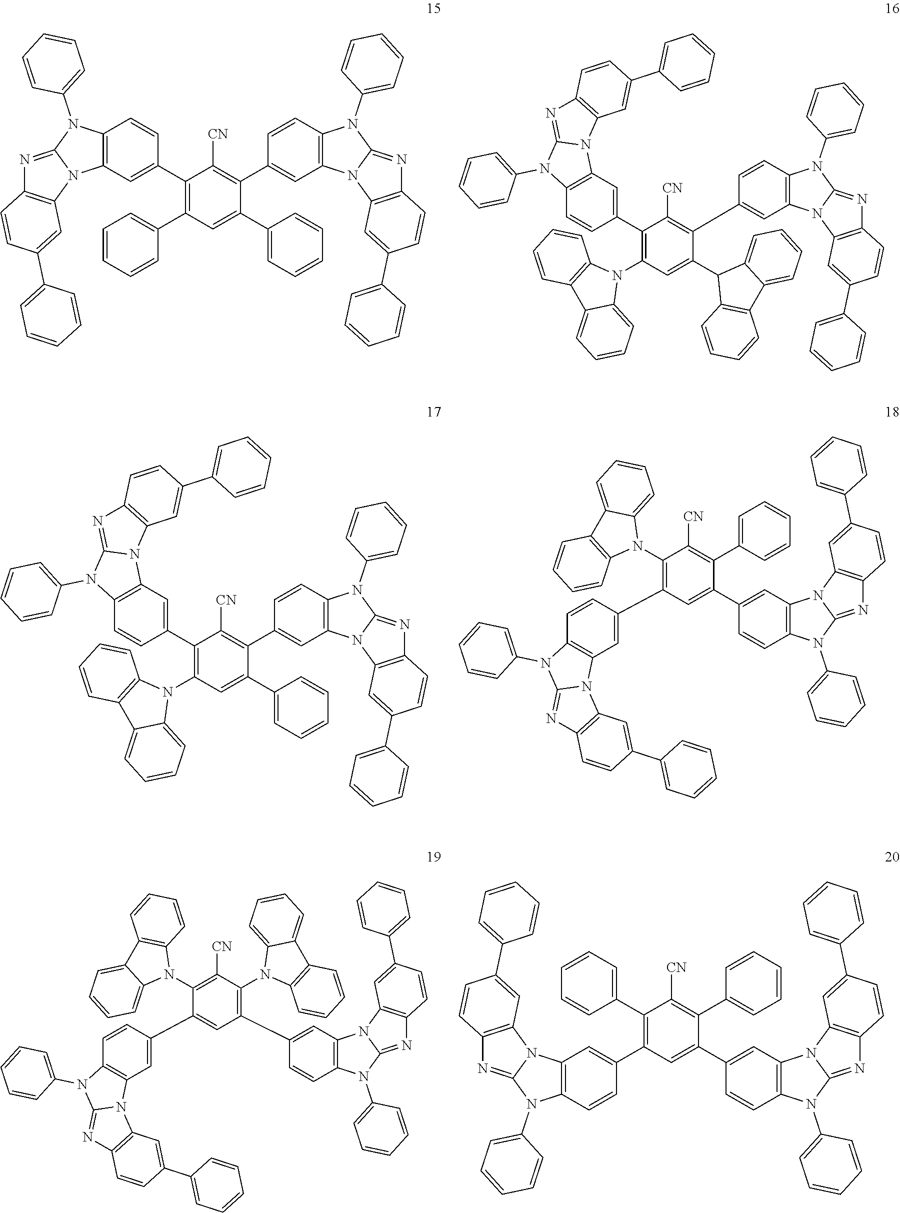

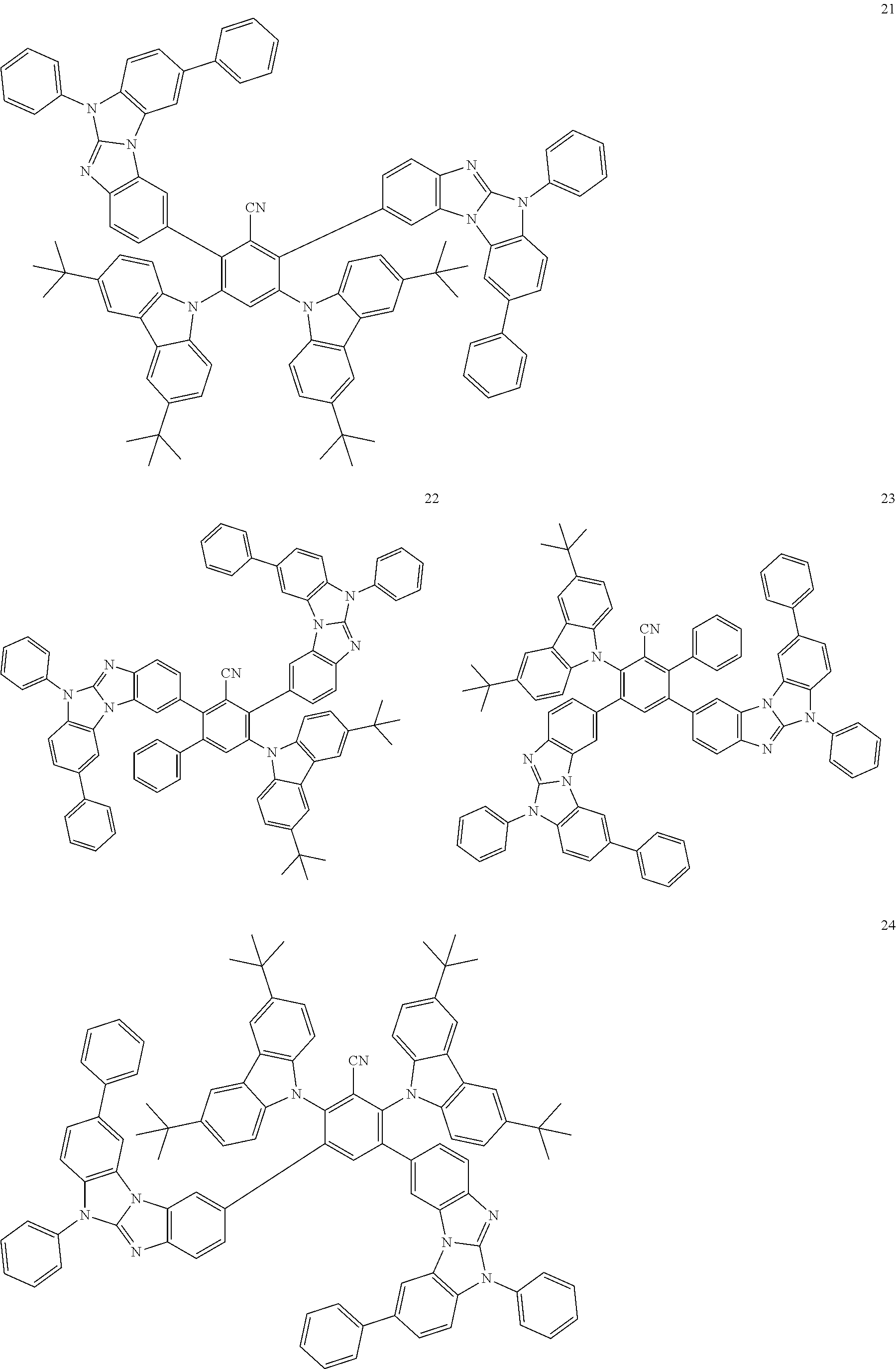

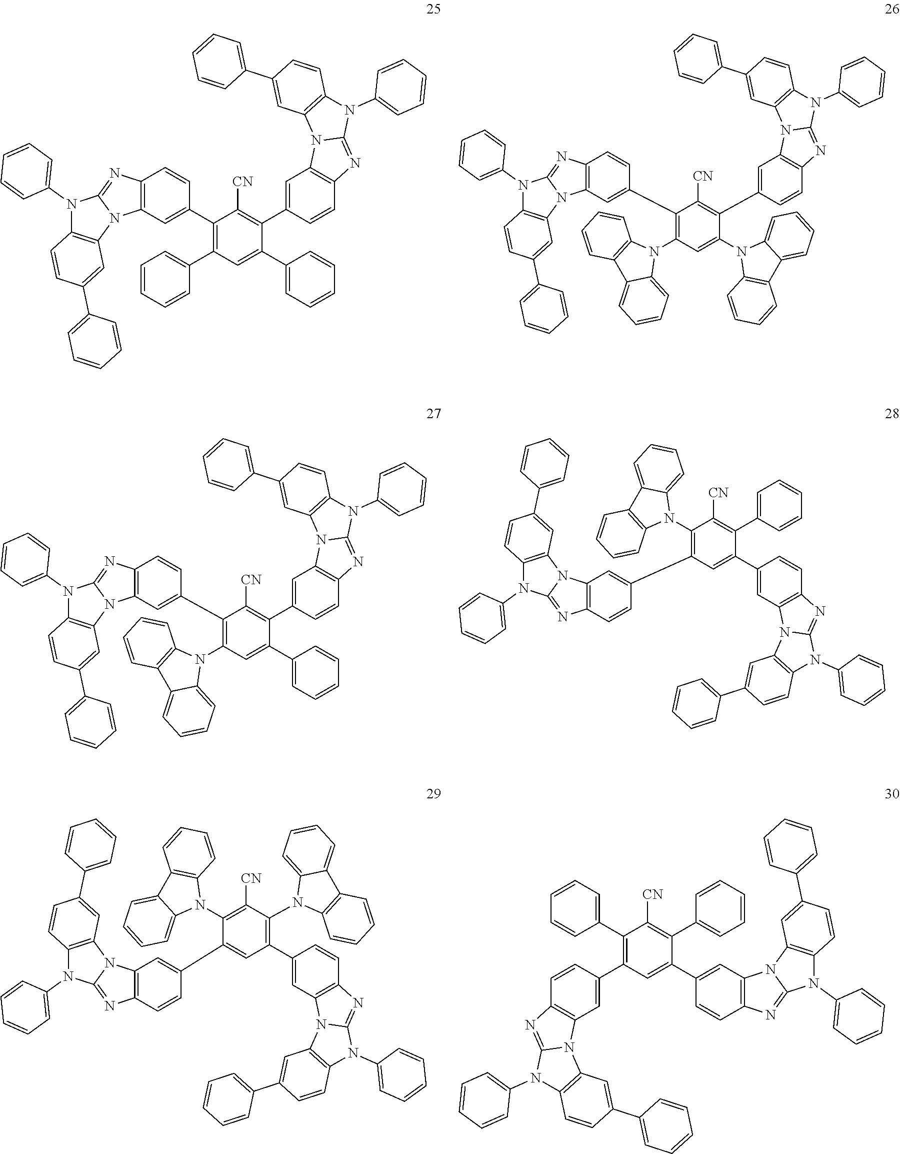

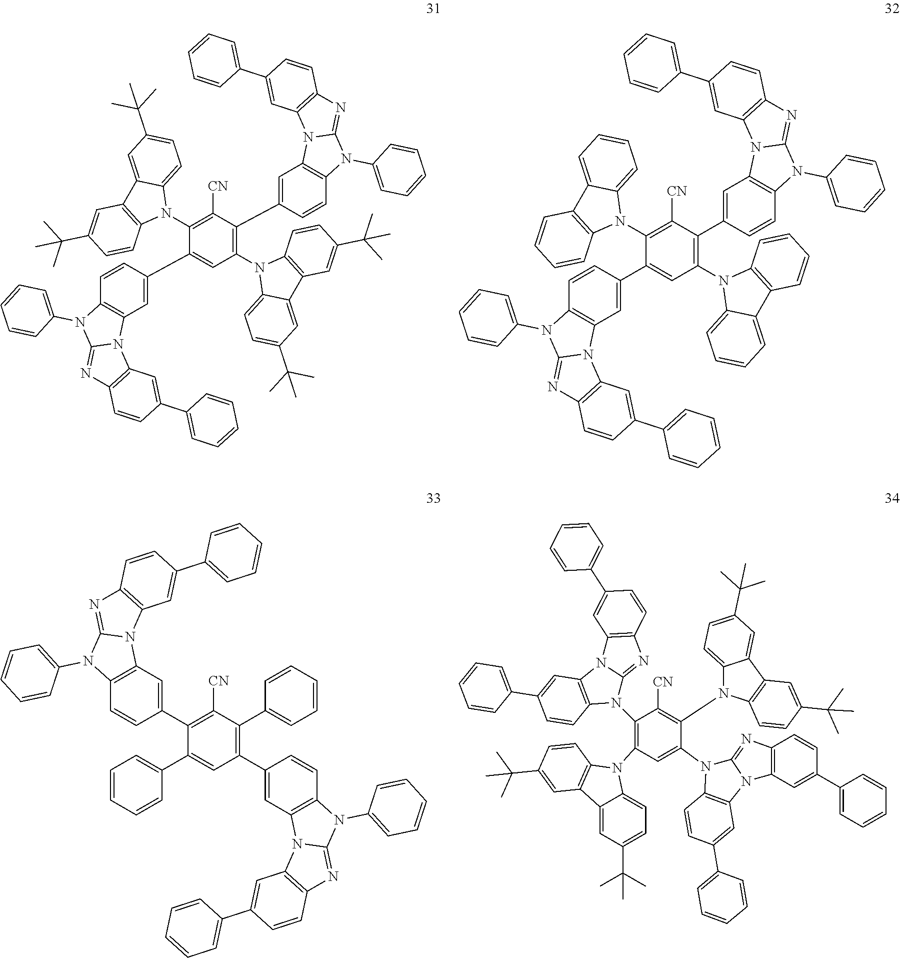

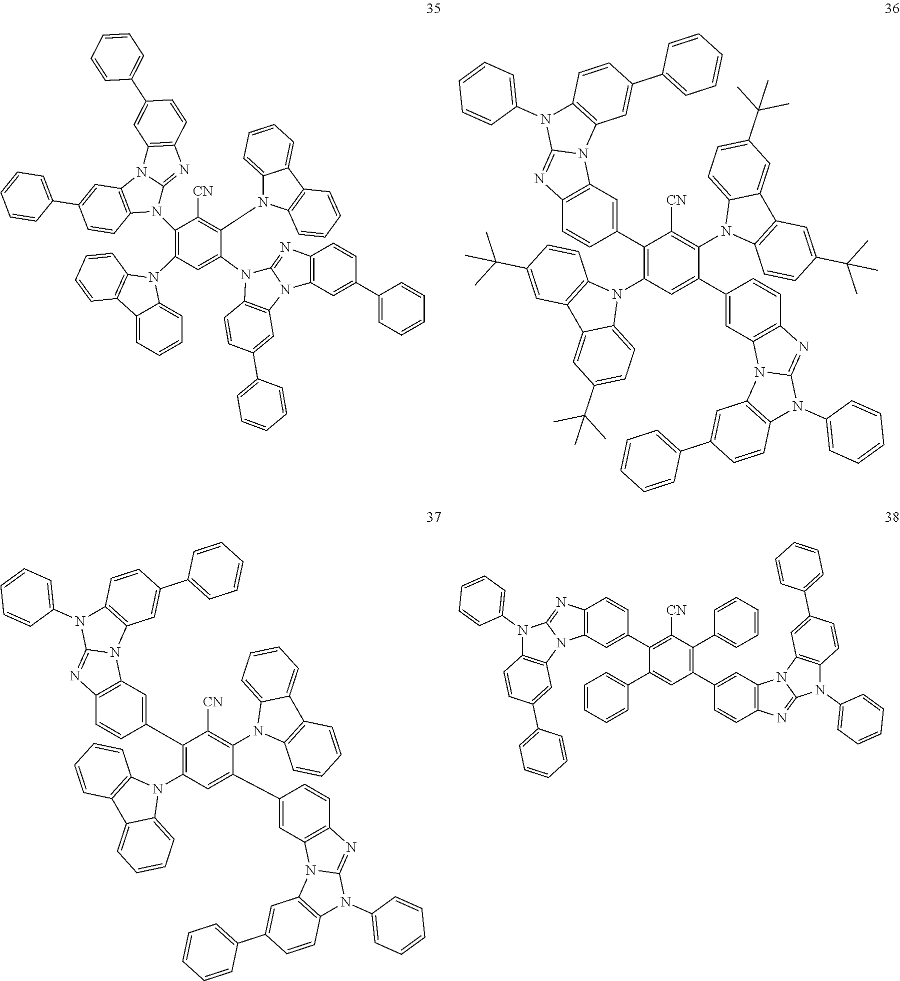

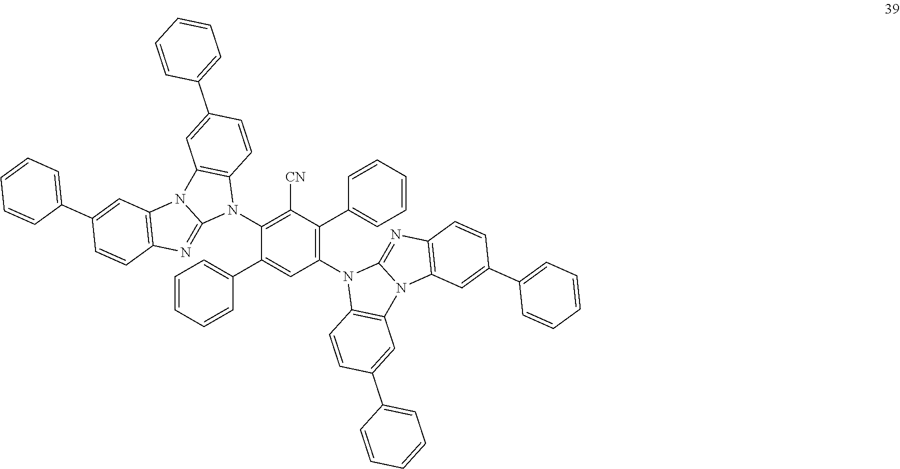 [SMILES: N#Cc1c(-c2ccccc2)c(-n2c3ccc(-c4ccccc4)cc3n3c4cc(-c5ccccc5)ccc4nc23)cc(-c2ccccc2)c1-n1c2ccc(-c3ccccc3)cc2n2c3cc(-c4ccccc4)ccc3nc12]

### Electron Transport Region in Organic Layer 15

An electron transport region may be located on the emission layer.

The electron transport region may include at least one a hole-blocking layer, an electron transport layer, an electron injection layer, or any combination thereof.

For example, the electron transport region may have the structure of electron transport layer, electron transport layer/electron injection layer, buffer layer/electron transport layer, hole-blocking layer/electron transport layer, buffer layer/electron transport layer/electron injection layer or hole-blocking layer/electron transport layer/electron injection layer, and embodiments of the present disclosure are not limited thereto. The electron transport region may further include an electron control layer. The electron transport layer may have a single-layered structure or a multi-layered structure including two or more different materials.

Conditions for forming the hole-blocking layer, the electron transport layer, and the electron injection layer which constitute the electron transport region may be understood by referring to the conditions for forming the hole injection layer.

The electron transport region may include a known electron transport material.

The electron transport region (for example, a buffer layer, a hole-blocking layer, an electron control layer, or an electron transport layer in the electron transport region) may include a metal-free compound containing at least one π electron-deficient nitrogen-containing cyclic group. The π electron-deficient nitrogen-containing cyclic group is the same as described above.

In an embodiment, the electron transport region may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a substituted or unsubstituted C₅-C₆₀ carbocyclic group or a substituted or unsubstituted C₁-C₆₀ heterocyclic group,

xe11 may be 1, 2, or 3,

xe1 is an integer from 0 to 5,

R₆₀₁ may be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic heterocondensed polycyclic group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and

xe21 is an integer from 1 to 5.

In one embodiment, at least one of Ar₆₀₁(s) in the number of xe11 and R₆₀₁(s) in the number of xe21 may include the π electron-deficient nitrogen-containing cyclic group.

In one embodiment, ring Ar₆₀₁ and L₆₀₁ in Formula 601 may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), —P(═O)(Q₃₁)(Q₃₂), or any combination thereof, wherein Q₃₁ to Q₃₃ may each independently be a C₁-C₁₀ alkyl group, a C₁-C₁₀ alkoxy group, a phenyl group, a biphenyl group, a terphenyl group, or a naphthyl group.

When xe11 in Formula 601 is 2 or more, two or more of Ar₆₀₁(s) may be linked to each other via a single bond.

In one or more embodiments, Ar₆₀₁ in Formula 601 may be an anthracene group.

In one or more embodiments, the compound represented by Formula 601 may be represented by Formula 601-1:

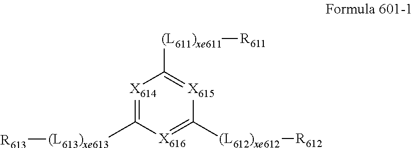

wherein, in Formula 601-1,

X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,

L₆₁₁ to L₆₁₃ may each independently be the same as described in connection with L₆₀₁,

xe611 to xe613 may each independently be the same as described in connection with xe1,

R₆₁₁ to R₆₁₃ may each independently be the same as described in connection with R₆₀₁, and

R₆₁₄ to R₆₁₆ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, or a naphthyl group.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group; or

—S(═O)₂(Q₆₀₁) or —P(═O)(Q₆₀₁)(Q₆₀₂),

wherein Q₆₀₁ and Q₆₀₂ are the same as described above.

When the electron transport region includes a hole-blocking layer, the hole-blocking layer may include, for example, at least one of BCP, Bphen, or any combination thereof, but embodiments of the present disclosure are not limited thereto.

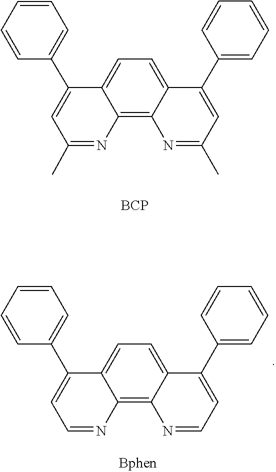 [SMILES: Cc1cc(-c2ccccc2)c2ccc3c(-c4ccccc4)cc(C)nc3c2n1 ; "BCP" ; c1ccc(-c2ccnc3c2ccc2c(-c4ccccc4)ccnc23)cc1]

A thickness of the hole-blocking layer may be in a range of about 20 Å to about 1,000 Å, for example, about 30 Å to about 300 Å. When the thickness of the hole-blocking layer is within any of these ranges, excellent hole blocking characteristics may be obtained without a substantial increase in driving voltage.

The electron transport layer may include at least one of 2,9-dimethyl-4,7-diphenyl-1,10-phenanthroline (BCP), 4,7-diphenyl-1,10-phenanthroline (Bphen), Alq₃, BAlq, 3-(biphenyl-4-yl)-5-(4-tert-butylphenyl)-4-phenyl-4H-1,2,4-triazole (TAZ), 2,2′,2″-(1,3,5-benzinetriyl)-tris(1-phenyl-1-H-benzimidazole) (TPBi), and NTAZ.

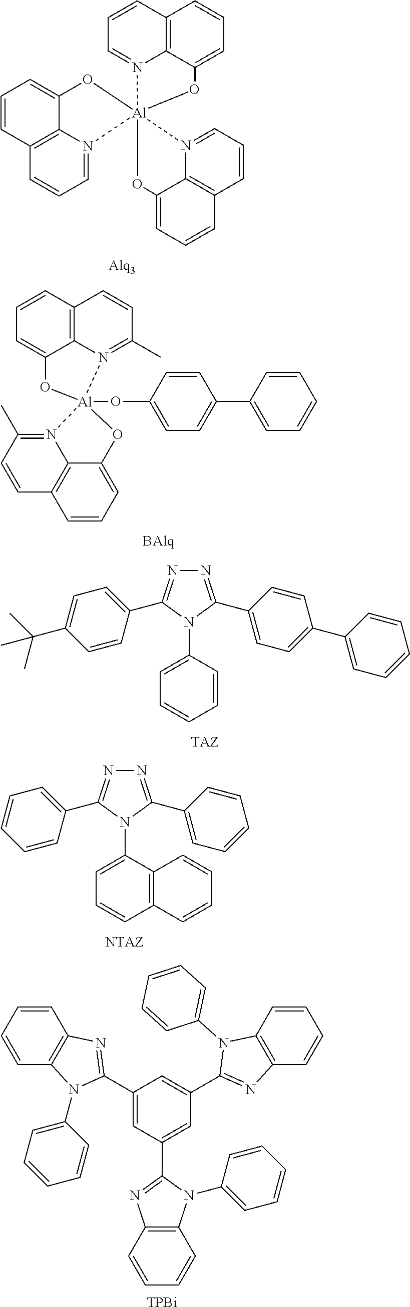

In one or more embodiments, the electron transport layer may include at least one of ET1 to ET25, but are not limited thereto:

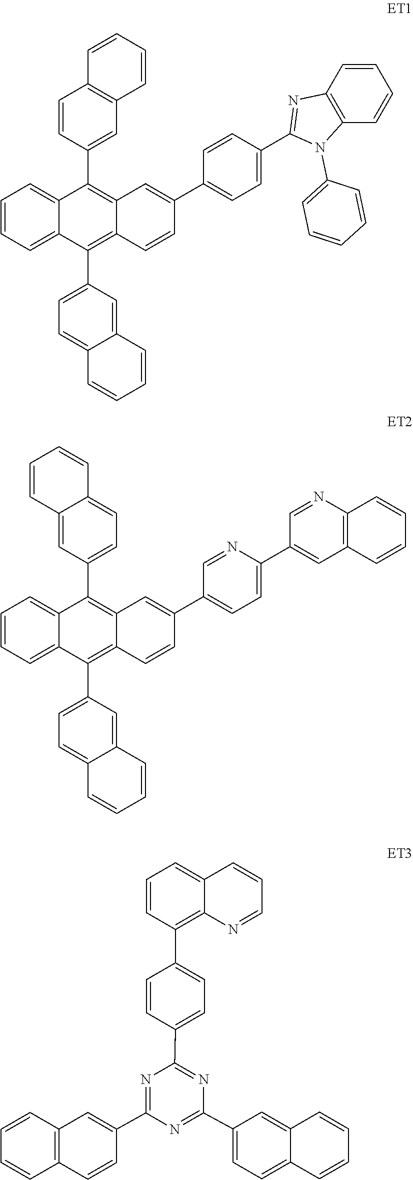 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

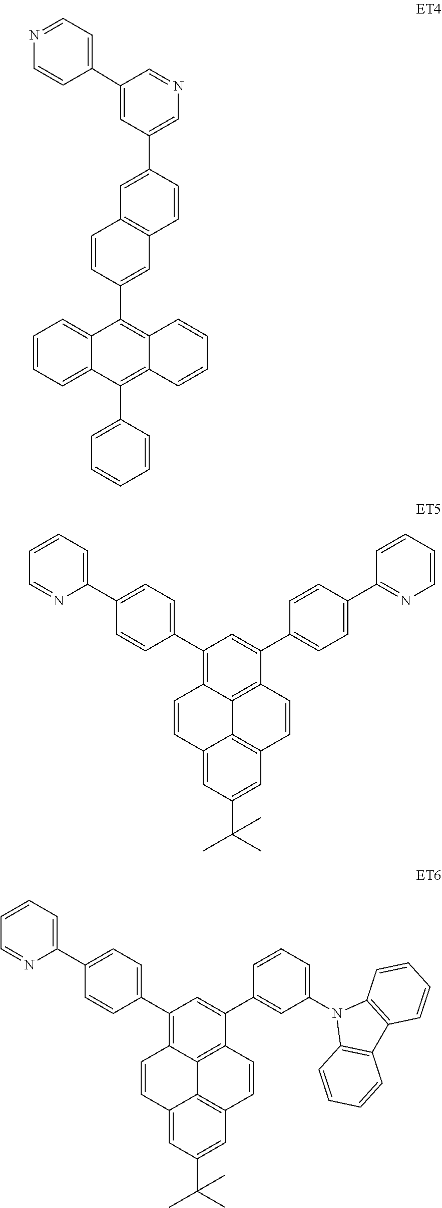 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

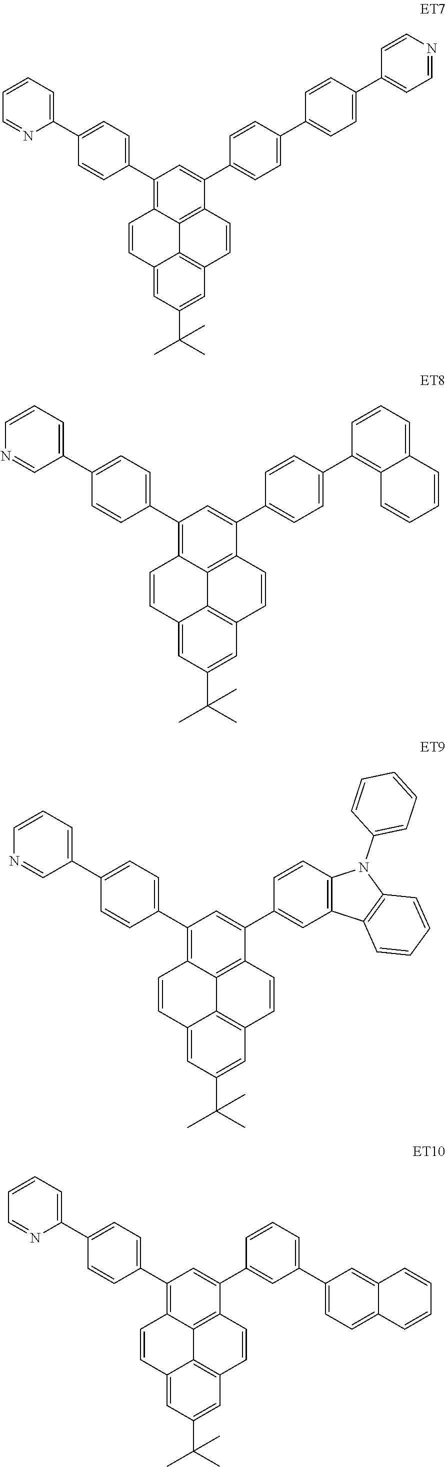 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34 ; CC(C)(C)c1cc2ccc3c(-c4ccc(-c5ccccn5)cc4)cc(-c4cccc(-c5ccc6ccccc6c5)c4)c4ccc(c1)c2c34]

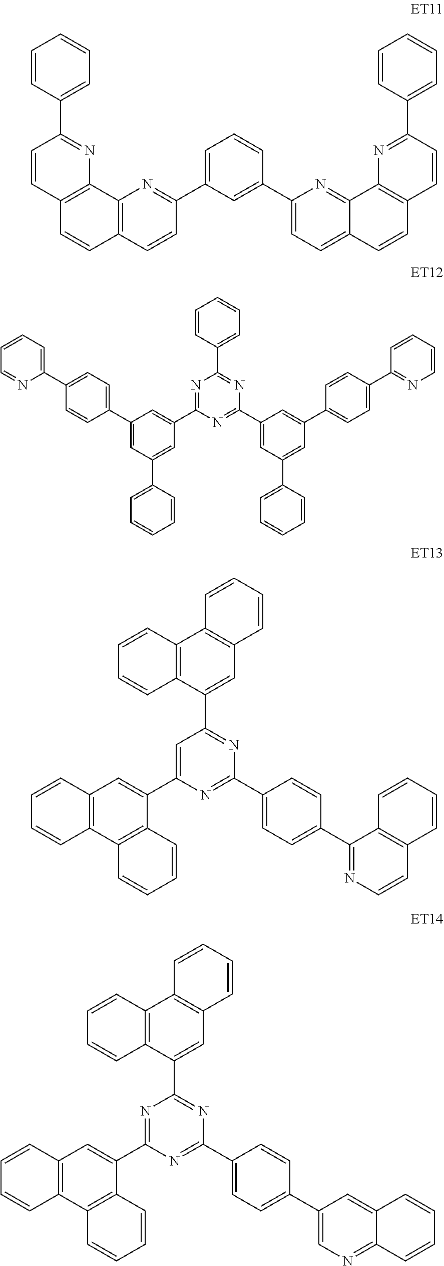 [SMILES: c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

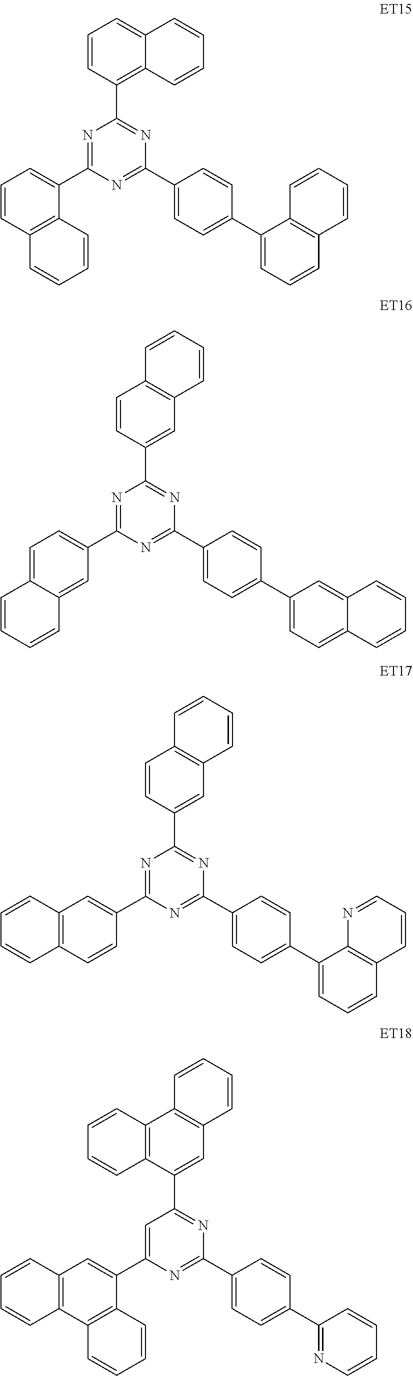 [SMILES: c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5ccc6ccccc6c5)nc(-c5ccc6ccccc6c5)n4)cc3)ccc2c1 ; c1ccc2cc(-c3nc(-c4ccc(-c5cccc6cccnc56)cc4)nc(-c4ccc5ccccc5c4)n3)ccc2c1 ; c1ccc(-c2ccc(-c3nc(-c4cc5ccccc5c5ccccc45)cc(-c4cc5ccccc5c5ccccc45)n3)cc2)nc1]

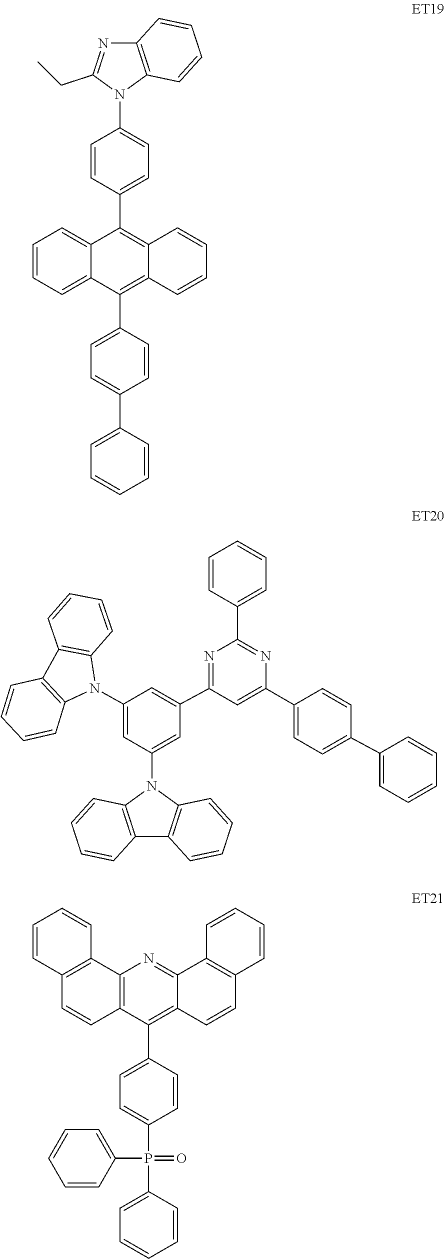 [SMILES: CCc1nc2ccccc2n1-c1ccc(-c2c3ccccc3c(-c3ccc(-c4ccccc4)cc3)c3ccccc23)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; O=P(c1ccccc1)(c1ccccc1)c1ccc(-c2c3ccc4ccccc4c3nc3c2ccc2ccccc23)cc1]

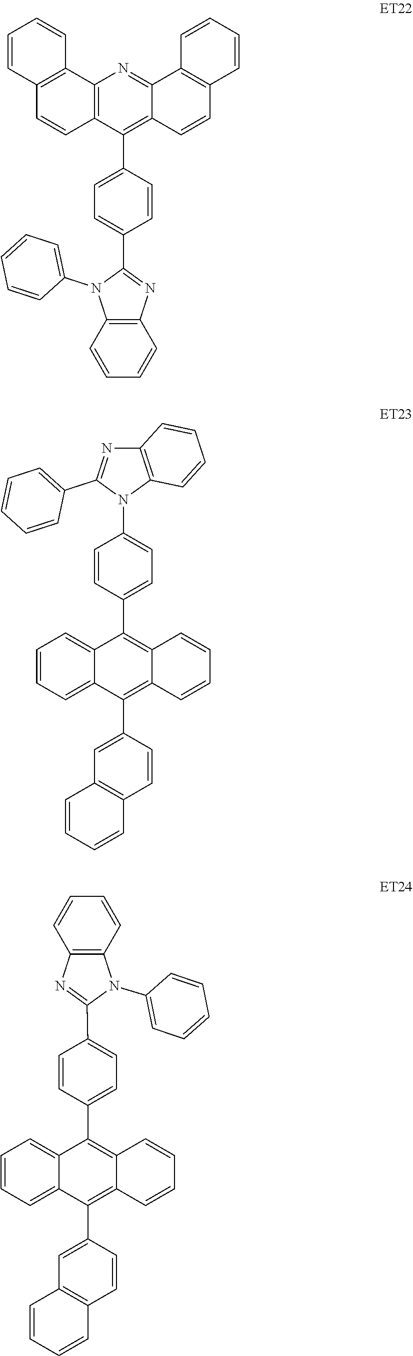 [SMILES: c1ccc(-n2c(-c3ccc(-c4c5ccc6ccccc6c5nc5c4ccc4ccccc45)cc3)nc3ccccc32)cc1 ; c1ccc(-c2nc3ccccc3n2-c2ccc(-c3c4ccccc4c(-c4ccc5ccccc5c4)c4ccccc34)cc2)cc1 ; c1ccc(-n2c(-c3ccc(-c4c5ccccc5c(-c5ccc6ccccc6c5)c5ccccc45)cc3)nc3ccccc32)cc1]

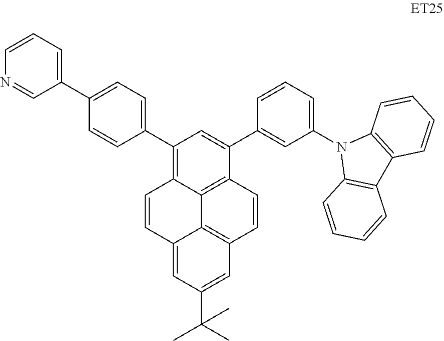 [SMILES: CC(C)(C)c1cc2ccc3c(-c4ccc(-c5cccnc5)cc4)cc(-c4cccc(-n5c6ccccc6c6ccccc65)c4)c4ccc(c1)c2c34]

A thickness of the electron transport layer may be in the range of about 100 Å to about 1,000 Å, for example, about 150 Å to about 500 Å. When the thickness of the electron transport layer is within the range described above, the electron transport layer may have satisfactory electron transporting characteristics without a substantial increase in driving voltage.

The electron transport layer may include a metal-containing material in addition to the material as described above.

The metal-containing material may include at least one alkali metal complex and alkaline earth-metal complex. The alkali metal complex may include a metal ion including a Li ion, a Na ion, a K ion, a Rb ion, or a Cs ion, and the alkaline earth-metal complex may include a metal ion including a Be ion, a Mg ion, a Ca ion, a Sr ion, or a Ba ion. A ligand coordinated with the metal ion of the alkali metal complex or the alkaline earth-metal complex may be a hydroxy quinoline, a hydroxy isoquinoline, a hydroxy benzoquinoline, a hydroxy acridine, a hydroxy phenanthridine, a hydroxy phenyloxazole, a hydroxy phenylthiazole, a hydroxy diphenyloxadiazole, a hydroxy diphenylthiadiazole, a hydroxy phenylpyridine, a hydroxy phenylbenzimidazole, a hydroxy phenylbenzothiazole, a bipyridine, a phenanthroline, and a cyclopentadiene, but embodiments of the present disclosure are not limited thereto.

The metal-containing material may include a Li complex. The Li complex may include, for example, Compound ET-D1 (lithium quinolate, LiQ) or ET-D2:

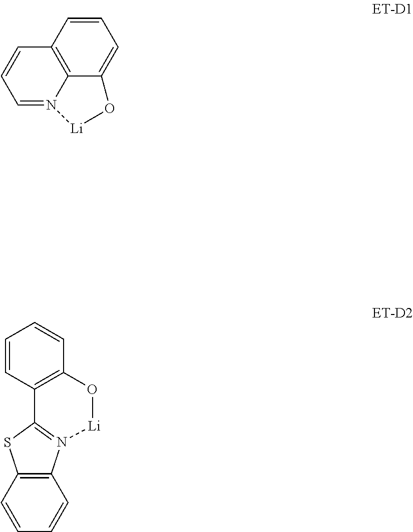

The electron transport region may include an electron injection layer that facilitates injection of electrons from the second electrode 19.

The electron injection layer may include at least one LiQ, LiF, NaCl, CsF, Li₂O, BaO, or any combination thereof.

A thickness of the electron injection layer may be in a range of about 1 Å to about 100 Å, and, for example, about 3 Å to about 90 Å. When the thickness of the electron injection layer is within the ranges described above, satisfactory electron injection characteristics may be obtained without a substantial increase in driving voltage.

The second electrode 19 is located on the organic layer 15. The second electrode 19 may be a cathode. A material for forming the second electrode 19 may be a metal, an alloy, an electrically conductive compound, or a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as the material for forming the second electrode 19. In one or more embodiments, to manufacture a top-emission type light-emitting device, a transmissive electrode formed using ITO or IZO may be used as the second electrode 19.

Hereinbefore, the organic light-emitting device has been described with reference to the figures, but embodiments of the present disclosure are not limited thereto.

### EXPLANATION OF TERMS

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” used herein refers to a monovalent group represented by —OA₁₀₁ (wherein A₁₀₁ is the C₁-C₆₀ alkyl group), and examples thereof include a methoxy group, an ethoxy group, and an isopropoxy group.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom of N, O, P, Si, S, Se, Ge, Te, B, or any combination thereof as a ring-forming atom and 1 to 10 carbon atoms, and non-limiting examples thereof include a tetrahydrofuranyl group and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom of N, O, P, Si, S, Se, Ge, Te, B, or any combination thereof as a ring-forming atom, 1 to 10 carbon atoms, and at least one carbon-carbon double bond in its ring. Examples of the C₁-C₁₀ heterocycloalkenyl group are a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a carbocyclic aromatic system that has at least one heteroatom N, O, P, Si, and S as a ring-forming atom, and 1 to 60 carbon atoms. The term “C₁-C₆₀ heteroarylene group” as used herein refers to a divalent group having a carbocyclic aromatic system that has at least one heteroatom of N, O, P, S, Se, Ge, Te, B, or any combination thereof as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₆-C₆₀ heteroaryl group and the C₆-C₆₀ heteroarylene group each include two or more rings, the rings may be fused to each other.

The term “C₆-C₆₀ aryloxy group” as used herein indicates —OA₁₀₂ (wherein A₁₀₂ is a C₆-C₆₀ aryl group), and the term “C₆-C₆₀ arylthio group” as used herein indicates —SA₁₀₃ (wherein A₁₀₃ is a C₆-C₆₀ aryl group).

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group (for example, having 2 to 60 carbon atoms) having two or more rings condensed with each other, a heteroatom of N, O, P, Si, S, Se, Ge, Te, B, or any combination thereof, other than carbon atoms, as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group described above.

The term “C₅-C₃₀ carbocyclic group” as used herein refers to a saturated or unsaturated cyclic group having, as a ring-forming atom, 5 to 30 carbon atoms only. The C₅-C₃₀ carbocyclic group may be a monocyclic group or a polycyclic group.

The term “C₁-C₃₀ heterocyclic group” as used herein refers to a saturated or unsaturated cyclic group having, as a ring-forming atom, at least one heteroatom of N, O, Si, P, S, Se, Ge, Te, B, or any combination thereof other than 1 to 30 carbon atoms. The C₁-C₃₀ heterocyclic group may be a monocyclic group or a polycyclic group.

As used herein, the number of carbons in each group that is substituted (e.g., C₁-C₆₀) excludes the number of carbons in the substituent. For example, a C₁-C₆₀ alkyl group can be substituted with a C₁-C₆₀ alkyl group. The total number of carbons included in the C₁-C₆₀ alkyl group substituted with the C₁-C₆₀ alkyl group is not limited to 60 carbons. In addition, more than one C₁-C₆₀ alkyl substituent may be present on the C₁-C₆₀ alkyl group. This definition is not limited to the C₁-C₆₀ alkyl group and applies to all substituted groups that recite a carbon range.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or any combination thereof;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or any combination thereof; or

—N(Q₃₁)(Q₃₂), —Si(Q₃₃)(Q₃₄)(Q₃₅), —B(Q₃₆)(Q₃₇), or —P(═O)(Q₃₈)(Q₃₉),

wherein Q₁ to Q, Q₁₁ to Q₁₉, Q₂₁ to Q₂₉, and Q₃₁ to Q₃₉ may each independently be: hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₁-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₁-C₆₀ heteroaryl group; a monovalent non-aromatic condensed polycyclic group; or a monovalent non-aromatic condensed heteropolycyclic group.

Hereinafter, a compound and an organic light-emitting device according to embodiments are described in detail with reference to Synthesis Example and Examples. However, the organic light-emitting device is not limited thereto. The wording “B was used instead of A” used in describing Synthesis Examples means that an amount of B used was identical to an amount of A used based on molar equivalence.

### EXAMPLES

### Synthesis Example 1: Synthesis of Compound 2362

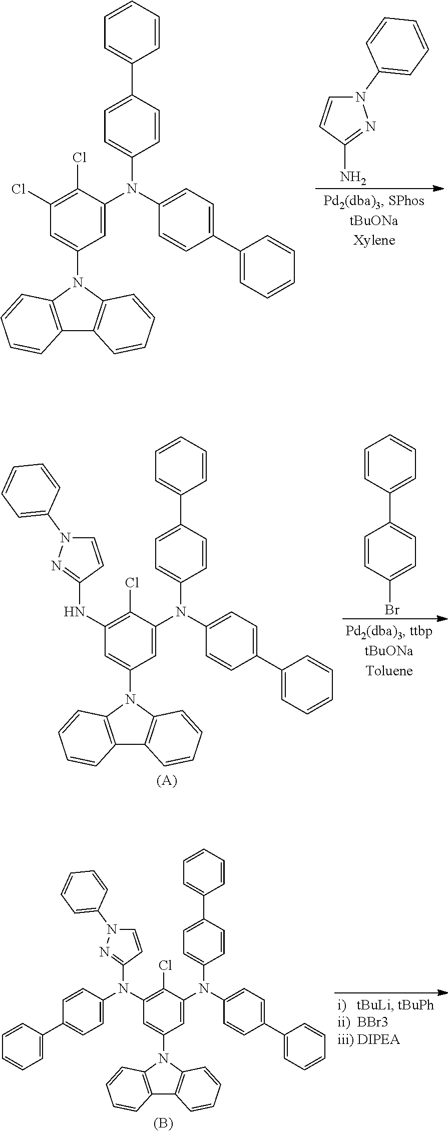

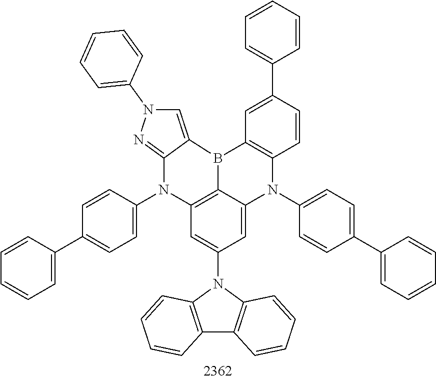

### Synthesis of Intermediate (A)

6.30 g (9.97 mmol) of N-(5-(9H-carbazol-9-yl)-2,3-dichlorophenyl)-N-([1,1′-biphenyl]-4-yl)-[1,1′-biphenyl]-4-amine, 2.06 g (13.0 mmol) of 1-phenyl-1H-pyrazol-3-amine, 0.457 g (0.499 mmol) of Pd₂(dba)₃, 0.409 g (0.996 mmol) of SPhos, and 1.44 g (15.0 mmol) of tBuONa were dissolved in 300 mL of xylene, and then, stirred for 7 hours at a temperature of 135° C. After completion of the reaction, the mixture was cooled to room temperature and diluted by adding 500 mL of ethyl acetate. The mixed solution was washed with 200 mL of a saturated ammonium chloride (NH₄Cl) aqueous solution and 200 mL of a saturated sodium chloride (NaCl) aqueous solution. The organic layer was dried using anhydrous magnesium sulfate (MgSO₄) and the resultant product was filtered and concentrated under reduced pressure. The product obtained was separated by silica gel column chromatography (ethyl acetate:n-hexane=1:4) to obtain the target compound, Intermediate (A), in an amount of 4.20 g (yield of 56%).

LC-Mass (calculated: 735.27 g/mol, found: M+1=736 g/mol)

### Synthesis of Intermediate (B)

2.70 g (3.58 mmol) of Intermediate (A), 1.00 g (4.30 mmol) of 4-bromo-1,1′-biphenyl, 0.206 g (0.358 mmol) of Pd(dba)₂, 0.147 g (0.358 mmol) of SPhos, and tBuONa 0.516 g (5.37 mmol) were dissolved in 120 mL of toluene, and then, stirred for 12 hours at a temperature of 100° C. After completion of the reaction, the mixture was cooled to room temperature and diluted by adding 200 mL of ethyl acetate. The mixed solution was washed with 150 mL of a saturated ammonium chloride (NH₄Cl) aqueous solution and 150 mL of a saturated sodium chloride (NaCl) aqueous solution. The organic layer was dried using anhydrous magnesium sulfate (MgSO₄) and the resultant product was filtered and concentrated under reduced pressure. The product obtained was separated by silica gel column chromatography (ethyl acetate:n-hexane=1:6) to obtain the target compound, Intermediate (B), in an amount of 1.10 g (yield of 41%).

LC-Mass (calculated: 905.33 g/mol, found: M+1=906 g/mol)

### Synthesis of Compound 2362

Intermediate (B) 0.896 g (0.988 mmol) was dissolved in 20 mL of t-butylbenzene and cooled to −78° C. 1.45 mL of t-BuLi (1.7 M solution in pentane, 2.47 mmol) was slowly added thereto and stirred at 40° C. for 1 hour. After cooling to −78° C. again, 0.190 mL (1.98 mmol) of BBr₃ was slowly added thereto, and stirred at room temperature for 1 hour. After the mixture was cooled to −78° C., 0.337 mL (1.98 mmol) of DIPEA was added slowly and stirred at 120° C. for 4 hours. The reaction mixture was cooled to 0° C., and 10 mL of methanol was added thereto to terminate the reaction. The solvent was removed through distillation under reduced pressure, and the obtained product was separated by silica gel column chromatography (ethyl acetate:n-hexane=1:4) to obtain 0.22 g (yield of 25%) of Compound 2362 as the target compound.

LC-Mass (calculated: 879.35 g/mol, found: M+1=880 g/mol)

### Synthesis Example 2: Synthesis of Compound 2361

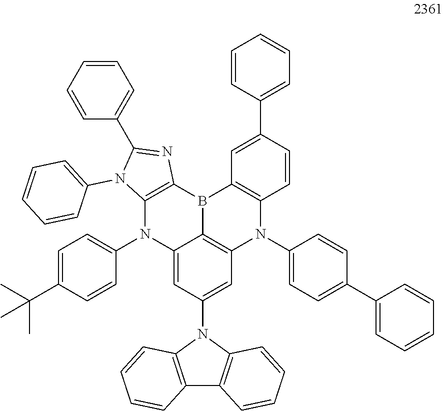 [SMILES: CC(C)(C)c1ccc(N2c3cc(-n4c5ccccc5c5ccccc54)cc4c3B(c3cc(-c5ccccc5)ccc3N4c3ccc(-c4ccccc4)cc3)c3nc(-c4ccccc4)n(-c4ccccc4)c32)cc1]

Compound 2361 was synthesized in the same manner as used to prepare Compound 2362, except that 4-(tert-butyl)aniline was used instead of 1-phenyl-1H-pyrazol-3-amine and 5-bromo-1,2-diphenyl-1H-imidazole was used instead of 4-bromo-1,1′-biphenyl.

LC-Mass (calculated: 935.42 g/mol, found: M+1=936 g/mol)

### Synthesis Example 3: Synthesis of Compound 37

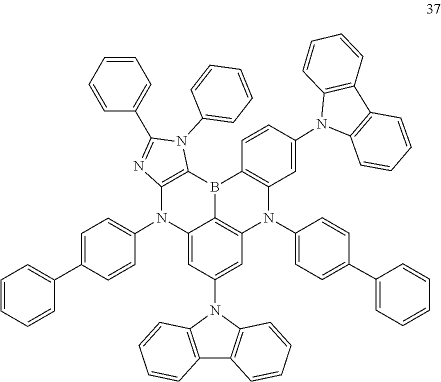 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1]

Compound was obtained in the same manner as used to obtain Compound 2361 in the same manner as used to synthesize Compound 2361, except that N-(5-(9H-carbazol-9-yl)-2,3-dichlorophenyl)-N-(3-(9H-carbazol-9-yl)phenyl)-[1,1′-biphenyl]-4-amine was used instead of N-(5-(9H-carbazol-9-yl)-2,3-dichlorophenyl)-N-([1,1′-biphenyl]-4-yl)-[1,1′-biphenyl]-4-amine, and [1,1′-biphenyl]-4-amine) was used instead of 4-(tert-butyl)aniline.

LC-Mass (calculated: 1044.41 g/mol, found: M+1=1045 g/mol)

### Synthesis Example 4: Synthesis of Compound 1968

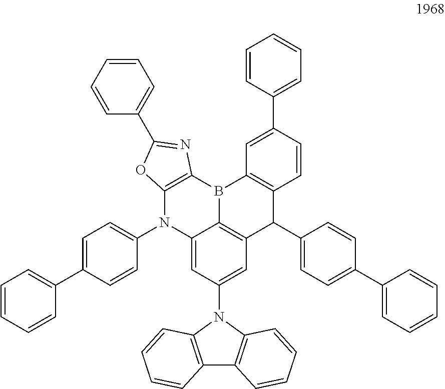 [SMILES: c1ccc(-c2ccc(C3c4ccc(-c5ccccc5)cc4B4c5nc(-c6ccccc6)oc5N(c5ccc(-c6ccccc6)cc5)c5cc(-n6c7ccccc7c7ccccc76)cc3c54)cc2)cc1]

Compound 1968 was synthesized in the same manner as used to synthesize Compound 2362, except that [1,1′-biphenyl]-4-amine was used instead of 1-phenyl-1H-pyrazol-3-amine, and 5-bromo-2-phenyloxazole was used instead of 4-bromo-1,1′-biphenyl.

LC-Mass (calculated: 880.34 g/mol, found: M+1=881 g/mol)

### Example 1-1

An ITO glass substrate was cut to a size of 50 mm×50 mm×0.5 mm and then, sonicated in acetone isopropyl alcohol and pure water, each for 15 minutes, and then, washed by exposure to ultraviolet (UV) light ozone for 30 minutes.

Then, HAT-CN was deposited on the ITO electrode (anode) on the glass substrate to form a hole injection layer having a thickness of 100 Å, NPB was deposited on the hole injection layer to form a first hole transport layer having a thickness of 500 Å, TCTA was deposited on the first hole transport layer to form a second hole transport layer having a thickness of 50 Å, and mCP was deposited on the second hole transport layer to form an electron-blocking layer having a thickness of 50 Å.

A first host (H1), a second host (H2) and an emitter (Compound 2362) were co-deposited on the electron-blocking layer to form an emission layer having a thickness of 400 Å. In this regard, the first host and the second host were mixed in a ratio of 60:40, and the emitter was adjusted to be 3 wt % based on the total weight of the first host, the second host, and the emitter.

DBFPO was deposited on the emission layer to form a hole-blocking layer having a thickness of 100 Å, and then DBFPO and Liq were co-deposited thereon at a weight ratio of 5:5 to form an electron transport layer having a thickness of 300 Å, and then, Liq was deposited on the electron transport layer to form an electron injection layer having a thickness of 10 Å, and Al was deposited on the electron injection layer to form a cathode having a thickness of 1000 Å, thereby completing the manufacture of an organic light-emitting device.

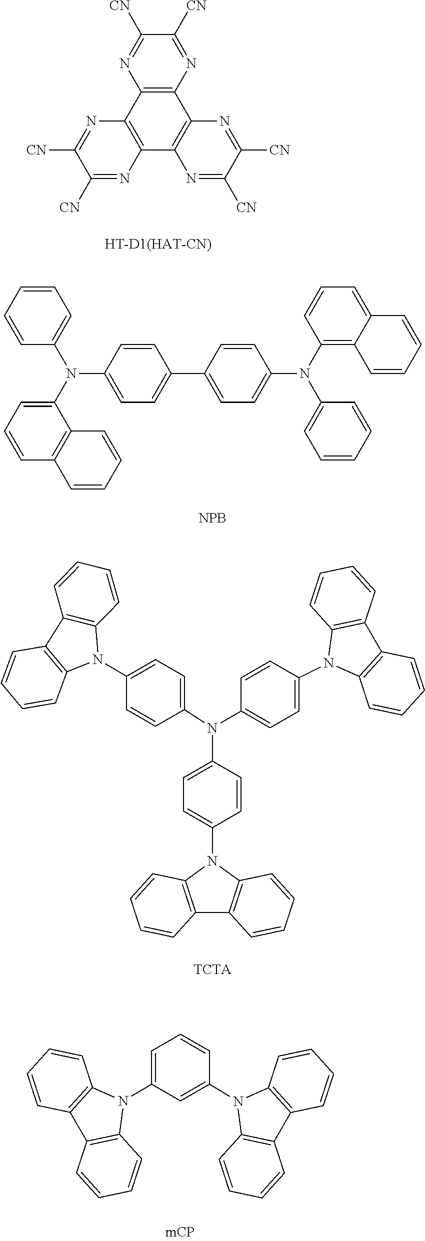

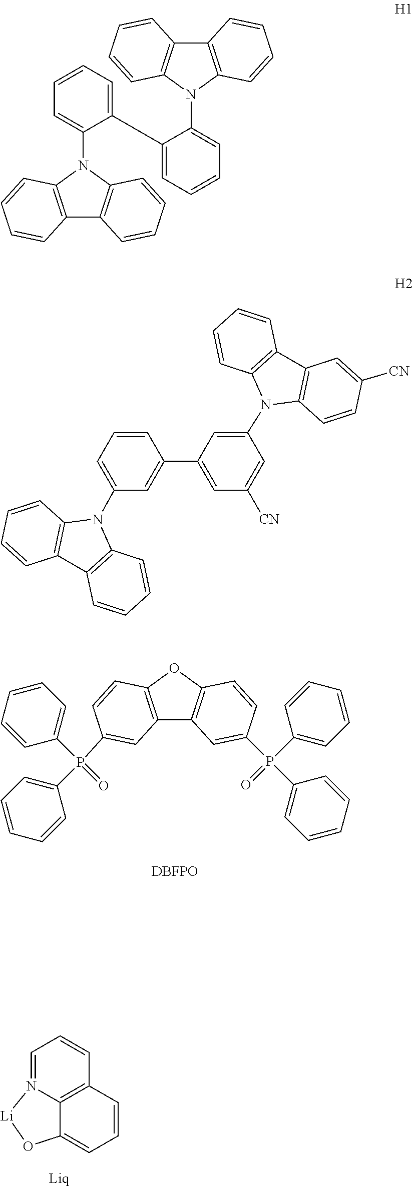

### Examples 1-2 to 1-4, and Comparative Example 1-1

Organic light-emitting devices were manufactured in the same manner as in Example 1-1 except that, the compounds shown in Table 8 were used instead of Compound 2362.

### Example 2-1

A glass substrate with an ITO electrode located thereon was cut to a size of 50 mm×50 mm×0.5 mm and then, sonicated in acetone isopropyl alcohol and pure water, each for 15 minutes, and then, washed by exposure to UV ozone for 30 minutes.

Then, HAT-CN was deposited on the ITO electrode (anode) on the glass substrate to form a hole injection layer having a thickness of 100 Å, NPB was deposited on the hole injection layer to form a first hole transport layer having a thickness of 500 Å, TCTA was deposited on the first hole transport layer to form a second hole transport layer having a thickness of 50 Å, and mCP was deposited on the second hole transport layer to form an electron-blocking layer having a thickness of 50 Å.

A first host (H1), a second host (H2), a sensitizer (S-1), and an emitter (Compound 2362) were co-deposited on the electron-blocking layer to form an emission layer having a thickness of 400 Å. At this time, the first host and the second host were mixed at a ratio of 60:40, and the amounts of the sensitizer and the emitter were adjusted to be 15 wt % and 1 wt %, respectively, based on the total weight of the first host, the second host, the sensitizer, and the emitter.

DBFPO was deposited on the emission layer to form a hole-blocking layer having a thickness of 100 Å, and then DBFPO and Liq were co-deposited thereon at a weight ratio of 5:5 to form an electron transport layer having a thickness of 300 Å, and then, Liq was deposited on the electron transport layer to form an electron injection layer having a thickness of 10 Å, and Al was deposited on the electron injection layer to form a cathode having a thickness of 1000 Å, thereby completing the manufacture of an organic light-emitting device.

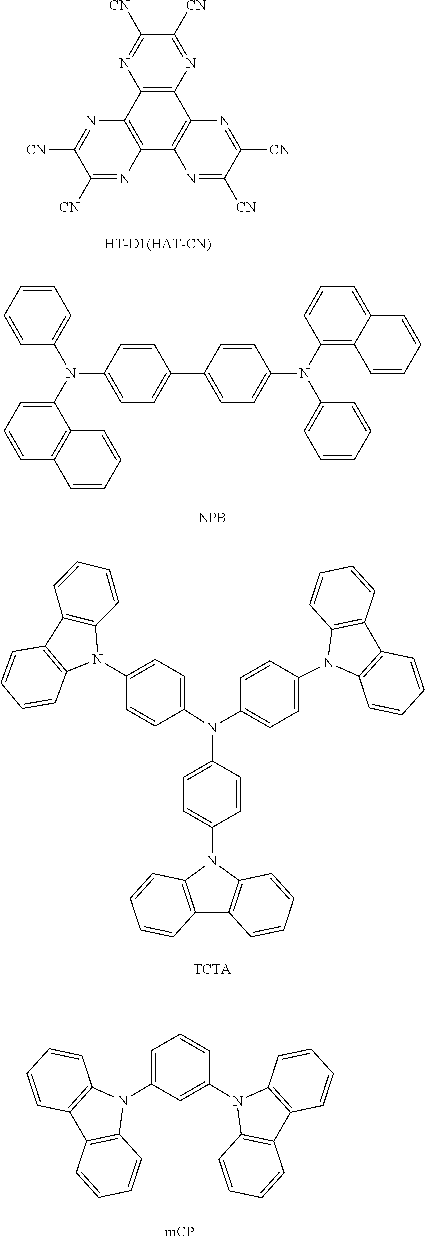

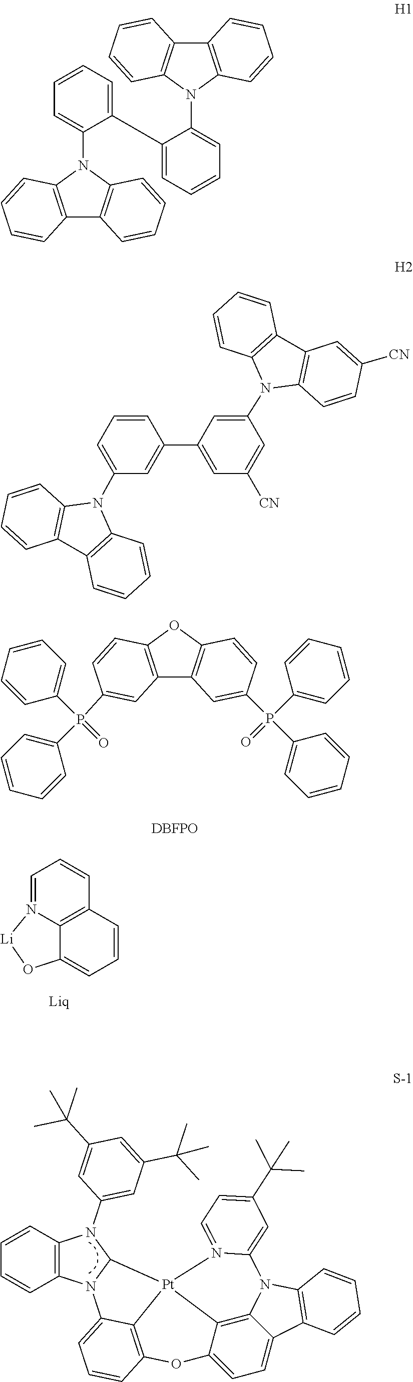

### Example 2-2 to 2-4 and Comparative Example 2-1

Organic light-emitting devices were manufactured in the same manner as in Example 2-1 except that, the compounds shown in Table 9 were used instead of Compound 2362.

### Evaluation Example 1: Characterization of Organic Light-Emitting Device

The driving voltage, maximum external quantum efficiency (EQE), and lifespan of the organic light-emitting devices prepared according to Examples 1-1 to 1-4 and Comparative Example 1-1 and Examples 2-1 to 2-4 and Comparative Example 2-1 were measured by using a current-voltmeter (Keithley 2400) and a luminance meter (Minolta Cs-1000A). Results thereof are shown in Table 8 and Table 9. The maximum external quantum efficiency and lifespan were described as relative values (%).

TABLE 8

| | | | | Lifespan |
|---|---|---|---|---|
| | | Driving | Maximum external | (LT₉₅) |
| | Compound | voltage | quantum efficiency | (Relative |
| | No. | (V) | (Relative value, %) | value, %) |
| Example 1-1 | 2362 | 4.87 | 146 | 231 |
| Example 1-2 | 2361 | 4.98 | 157 | 172 |
| Example 1-3 | 37 | 4.77 | 125 | 190 |
| Example 1-4 | 1968 | 5.02 | 108 | 143 |
| Comparative | C1 | 5.56 | 100 | 100 |
| Example 1-1 | | | | |

TABLE 9

| | | | | Lifespan |
|---|---|---|---|---|
| | | Driving | Maximum external | (LT₉₅) |
| | Compound | voltage | quantum efficiency | (Relative |
| | No. | (V) | (Relative value, %) | value, %) |
| Example 2-1 | 2362 | 4.32 | 115 | 157 |
| Example 2-2 | 2361 | 4.44 | 118 | 124 |
| Example 2-3 | 37 | 4.21 | 108 | 130 |
| Example 2-4 | 1968 | 1968 | 103 | 111 |
| Comparative | C1 | 4.59 | 100 | 100 |
| Example 2-1 | | | | |

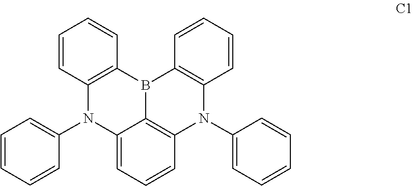 [SMILES: c1ccc(N2c3ccccc3B3c4ccccc4N(c4ccccc4)c4cccc2c43)cc1]

From Table 8, it can be confirmed that the organic light-emitting devices according to Examples 1-1 to 1-4 have better characteristics than the organic light-emitting device according to Comparative Example 1-1 in terms of a driving voltage, maximum external quantum efficiency, and lifespan characteristics.

From Table 9, it can be confirmed that the organic light-emitting devices according to Examples 2-1 to 2-4 have better characteristics than the organic light-emitting device according to Comparative Example 2-1 in terms of a driving voltage, maximum external quantum efficiency, and lifespan characteristics.

By using the condensed cyclic compound, an organic light-emitting device having high efficiency and high color purity and an electronic apparatus including the same can be provided.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A condensed cyclic compound represented by Formula 1: 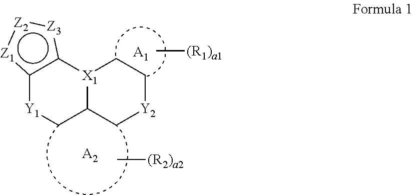
wherein, in Formula 1,
X₁ is B, N, P, or P(═O),
Z₁ to Z₃ are each independently O, S, N, N(R₃), or C(R₄), wherein at least two of Z₁ to Z₃ are each independently O, S, N, or N(R₃),
the ring including Z₁ to Z₃ is a 5-membered heteroaromatic ring,
Y₁ and Y₂ are each independently N[(L₁₁)_{b11}-(R₅)ₐ₅], O, S, Se, B(R₆), or C(═O), Y₁ and Y₂ are identical to or different from each other,
A₁ and A₂ are each independently a C₅-C₃₀ carbocyclic group or a C₁-C₃₀ heterocyclic group,
L₁₁ is a substituted or unsubstituted C₅-C₃₀ carbocyclic group or a substituted or unsubstituted C₁-C₃₀ heterocyclic group,
b11 is 0, 1, or 2,
R₁ to R₆ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),
a1, a2, and a5 are each independently an integer from 1 to 10,
a substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or any combination thereof;
—N(Q₃₁)(Q₃₂), —Si(Q₃₃)(Q₃₄)(Q₃₅), —B(Q₃₆)(Q₃₇), or —P(═O)(Q₃₈)(Q₃₉); or
any combination thereof,
wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₁-C₆₀ alkyl group which is unsubstituted or substituted with deuterium, a C₁-C₆₀ alkyl group, a C₆-C₆₀ aryl group, or any combination thereof, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group which is unsubstituted or substituted with deuterium, a C₁-C₆₀ alkyl group, a C₆-C₆₀ aryl group, or any combination thereof, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group.

2. The condensed cyclic compound of claim 1, wherein
the ring including Z₁ to Z₃ is an imidazole group, an oxazole group, a thiazole group, a pyrazole group, an isoxazole group, an isothiazole group, a triazole group, an oxadiazole group, or a thiadiazole group.

3. The condensed cyclic compound of claim 1, wherein
the ring including Z₁ to Z₃ is one of the groups represented by Formulae A3-1 to A3-27: 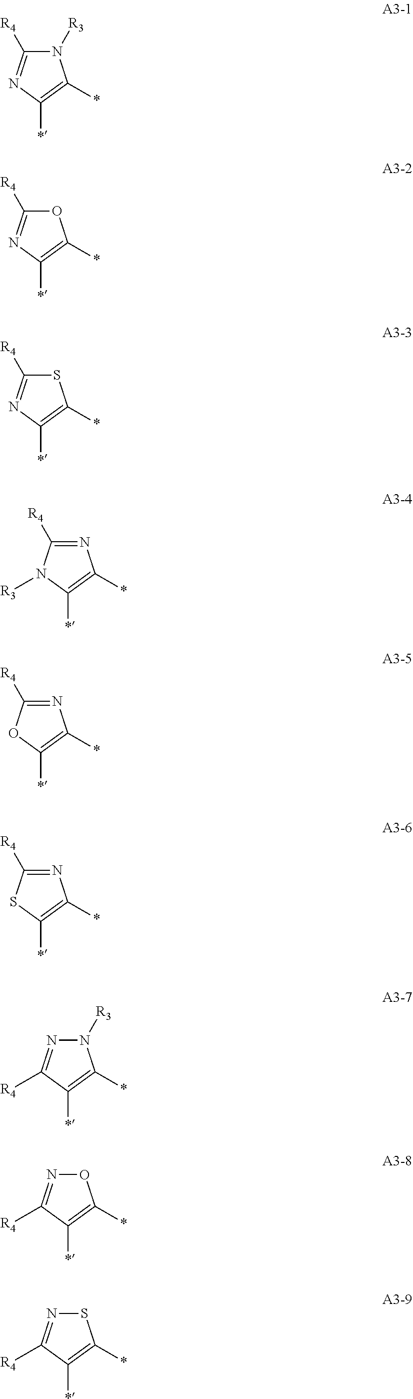 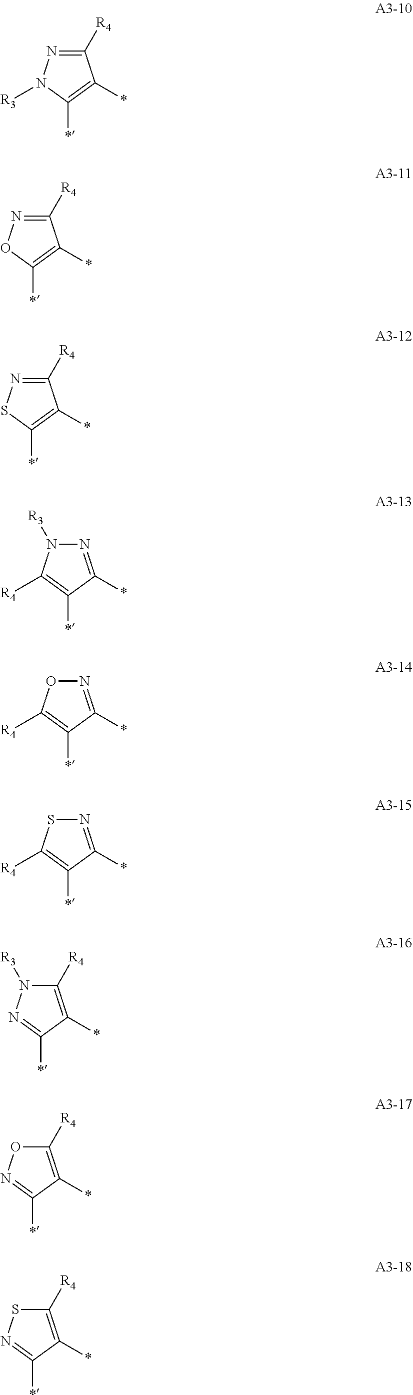 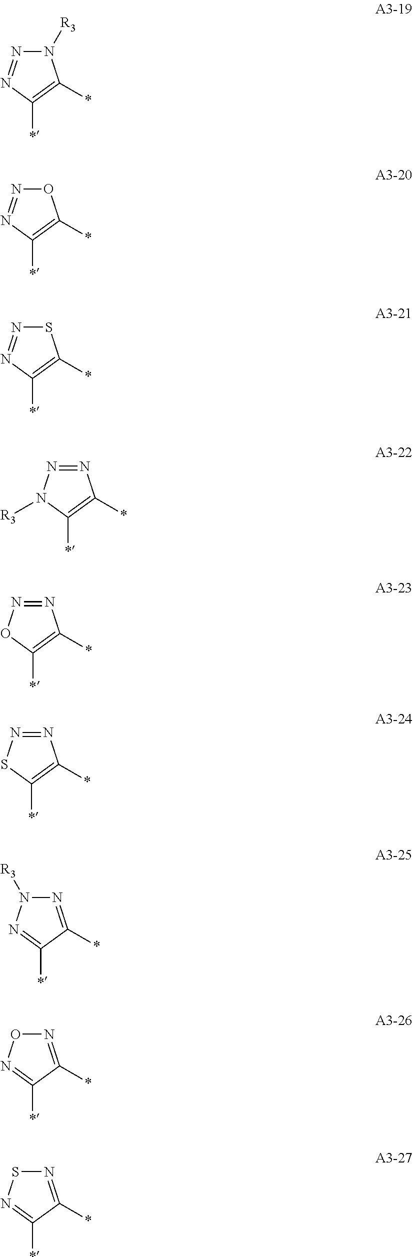
wherein, in Formulae A3-1 to A3-27,
R₃ and R₄ are each the same as described in connection with claim 1,
* indicates a binding site to X₁, and
*′ indicates a binding site to Y₁.

4. The condensed cyclic compound of claim 3, wherein
R₃ is a cyclopentyl group, a cyclohexyl group, a cyclopentyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, or a dibenzothiophenyl group, each unsubstituted or substituted with deuterium, —F, a cyano group, a C₁-C₂₀ alkyl group, a C₁-C₂₀ alkoxy group, a cyclopentyl group, a cyclohexyl group, a cyclopentyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof,
R₄ is hydrogen, deuterium, —F, or a cyano group; or
a C₁-C₂₀ alkyl group or a C₁-C₂₀ alkoxy group, each unsubstituted or substituted with deuterium, —F, a cyano group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof; or
a cyclopentyl group, a cyclohexyl group, a cyclopentyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, or a dibenzothiophenyl group, each unsubstituted or substituted with deuterium, —F, a cyano group, a C₁-C₂₀ alkyl group, a C₁-C₂₀ alkoxy group, a cyclopentyl group, a cyclohexyl group, a cyclopentyl group, a cyclohexenyl group, a cycloheptenyl group, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, or any combination thereof.

5. The condensed cyclic compound of claim 1, wherein
X₁ is B, and Y₁ and Y₂ are each independently N[(L₁₁)_{b11}-(R₅)ₐ₅], O, S, Se, or C(═O),
X₁ is P or P(═O), and Y₁ and Y₂ are each independently N[(L₁₁)_{b11}-(R₅)ₐ₅], or
X₁ is N, and Y₁ and Y₂ are each independently N[(L₁₁)_{b11}-(R₅)ₐ₅], B(R₆), or C(═O).

6. The condensed cyclic compound of claim 1, wherein
A₁ and A₂ are each independently a benzene group, a naphthalene group, a 1,2,3,4-tetrahydronaphthalene group, a fluorene group, a carbazole group, a benzofuran group, a dibenzofuran group, a benzothiophene group, a dibenzothiophene group, a benzosilole group, a dibenzosilole group, a pyridine group, a pyrimidine group, a pyrazine group, a pyridazine group, a quinoline group, an isoquinoline group, a quinoxaline group, a quinazoline group, an imidazole group, an oxazole group, a thiazole group, a pyrazole group, an isoxazole group, an isothiazole group, a triazole group, an oxadiazole, or a thiadiazole group.

7. The condensed cyclic compound of claim 1, wherein
the condensed cyclic compound is represented by Formula 1A or 1B: 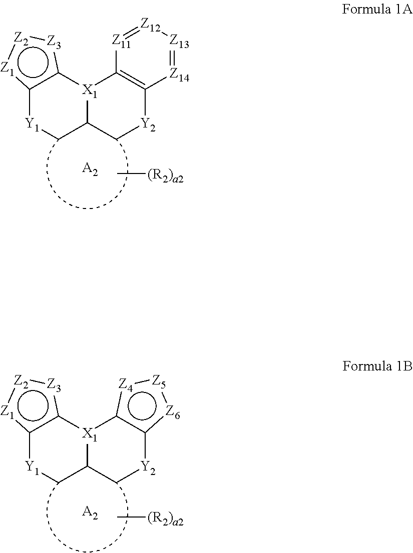
wherein, in Formulae 1A and 1B,
Z₁₁ is C(R₁₁) or N, Z₁₂ is C(R₁₂) or N, Z₁₃ is C(R₁₃) or N, Z₁₄ is C(R₁₄) or N,
Z₄ to Z₆ are each independently O, S, N, N(R₁₁), or C(R₁₂), wherein at least two of Z₄ to Z₆ are each independently O, S, N, or N(R₁₁),
the ring including Z₄ to Z₆ is a 5-membered heteroaromatic ring,
R₁₁ to R₁₄ are each the same as described in connection with R₁ in claim 1, and
X₁, Z₁ to Z₃, Y₁, Y₂, A₂, R₂, and a2 are each the same as described in connection with claim 1.

8. The condensed cyclic compound of claim 7, wherein
the ring including Z₄ to Z₆ in Formula 1-2 is a group represented by Formula A1-1 to A1-27: 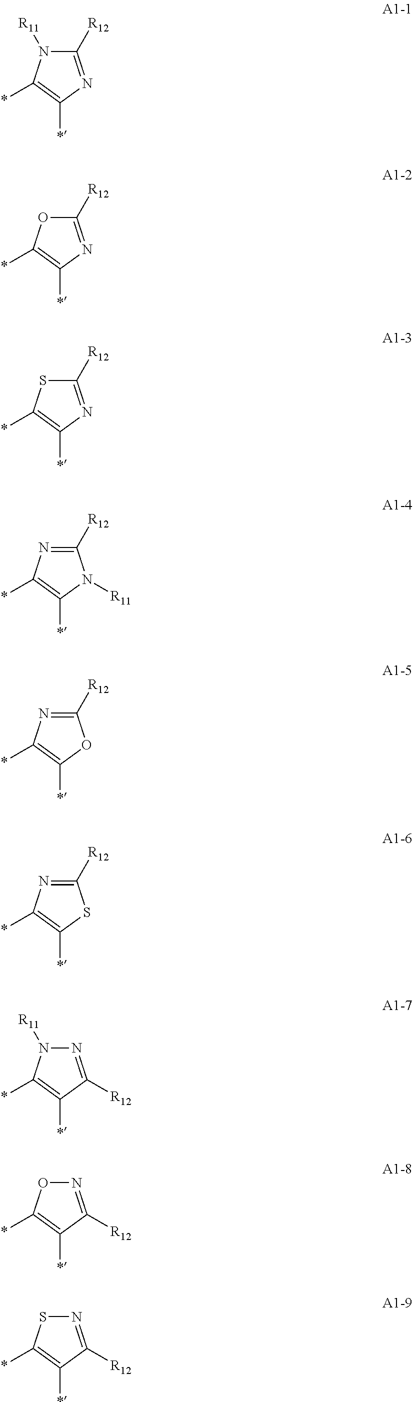 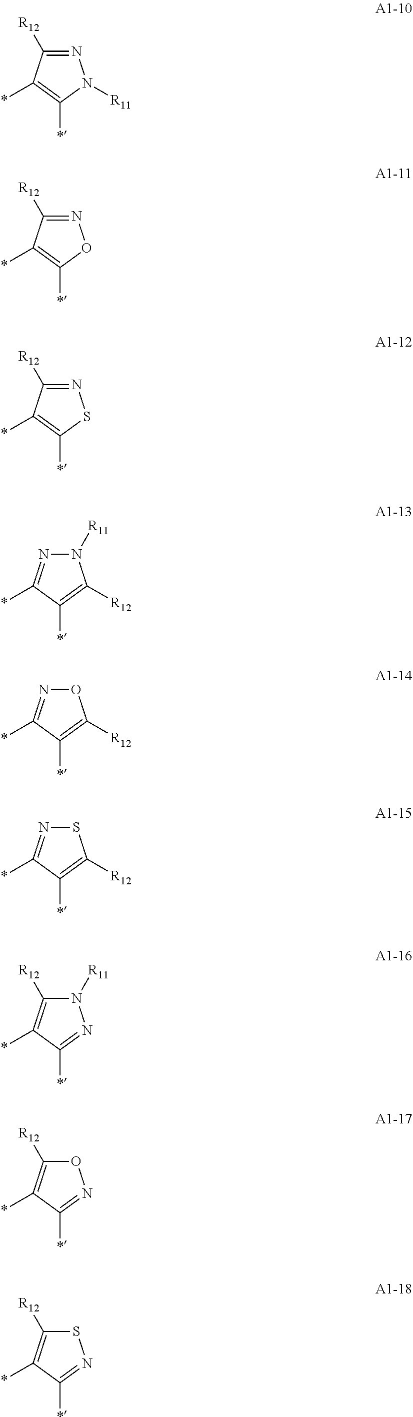 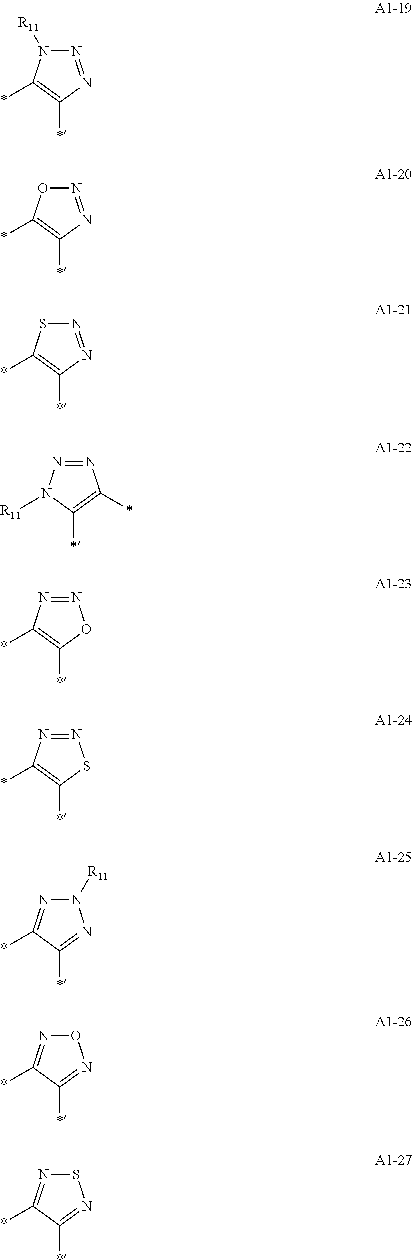
wherein, in Formulae A1-1 to A1-27,
R₁₁ and R₁₂ are each the same as described in claim 7,
* indicates a binding site to X₁, and
*′ indicates a binding site to Y₂.

9. The condensed cyclic compound of claim 1, wherein
R₁ to R₆ are each independently:
hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof,
a C₁-C₂₀ alkyl group or a C₁-C₂₀ alkoxy group, each unsubstituted or substituted with deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a phenyl group, a biphenyl group, a terphenyl group, a naphthyl group, a fluorenyl group, a pyridinyl group, a pyrimidinyl group, a triazinyl group, a quinolinyl group, an isoquinolinyl group, a carbazolyl group, a dibenzofuranyl group, a dibenzothiophenyl group, —N(Q₃₄)(Q₃₅), or any combination thereof;
a cyclopentyl group, a cyclohexyl group, a cyclopentenyl group, a cyclohexenyl group, a cycloheptenyl group, a phenyl group, a naphthyl group, a pentalenyl group, an indenyl group, an azulenyl group, a heptalenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a pyrrolyl group, an imidazolyl group, a pyrazolyl group, a pyridinyl group, a pyrazinyl group, a pyrimidinyl group, a pyridazinyl group, a triazinyl group, an indolyl group, an isoindolyl group, a benzimidazolyl group, an indazolyl group, a carbazolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a furanyl group, a thiophenyl group, an oxazolyl group, an isoxazolyl group, a thiazolyl group, an isothiazolyl group, a triazolyl group, a tetrazolyl group, an oxadiazolyl group, a thiadiazolyl group, a silolyl group, a benzofuranyl group, a benzothiophenyl group, a benzoxazolyl group, a benzothiazolyl group, a benzosilolyl group, a dibenzofuranyl group, a dibenzothiophenyl group, or a dibenzosilolyl group, each unsubstituted or substituted with deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cyclopentenyl group, a cyclohexenyl group, a cycloheptenyl group, a phenyl group, a naphthyl group, a pentalenyl group, an indenyl group, an azulenyl group, a heptalenyl group, an acenaphthyl group, a fluorenyl group, a spiro-bifluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a pyrrolyl group, an imidazolyl group, a pyrazolyl group, a pyridinyl group, a pyrazinyl group, a pyrimidinyl group, a pyridazinyl group, a triazinyl group, an indolyl group, an isoindolyl group, a benzimidazolyl group, an indazolyl group, a carbazolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a furanyl group, a thiophenyl group, an oxazolyl group, an isoxazolyl group, a thiazolyl group, an isothiazolyl group, a triazolyl group, a tetrazolyl group, an oxadiazolyl group, a thiadiazolyl group, a silolyl group, a benzofuranyl group, a benzothiophenyl group, a benzoxazolyl group, a benzothiazolyl group, a benzosilolyl group, a dibenzofuranyl group, a dibenzothiophenyl group, a dibenzosilolyl group, —N(Q₃₄)(Q₃₅), or any combination thereof; or
—N(Q₁)(Q₂),
wherein Q₁, Q₂, Q₃₄, and Q₃₅ are each independently: a C₁-C₁₀ alkyl group; a C₁-C₁₀ alkoxy group; a phenyl group, a biphenyl group, a terphenyl group, or a naphthyl group, each unsubstituted or substituted with deuterium, a C₁-C₁₀ alkyl group, a phenyl group, or any combination thereof.

10. The condensed cyclic compound of claim 1, wherein
at least one of R₁(s) in the number of a1 and R₂(s) in the number of a2 is a group represented by Formula 2A or 2B: 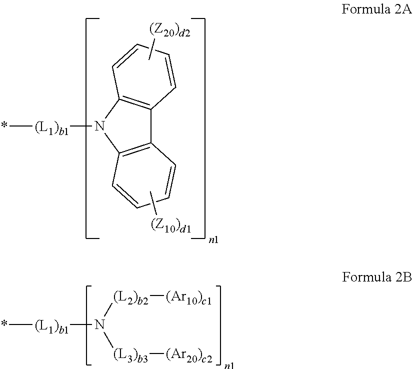
wherein, in Formulae 2A and 2B,
L₁ to L₃, Ar₁₀ and Ar₂₀ are each independently a substituted or unsubstituted C₅-C₆₀ carbocyclic group, or a substituted or unsubstituted C₁-C₆₀ heterocyclic group,
b1 to b3 are each independently 0, 1, or 2,
c1 and c2 are each independently an integer from 1 to 5,
Z₁₀ and Z₂₀ are each independently a deuterium, —F, —Cl, —Br, —I, —SF₅, a hydroxyl group, a cyano group, a nitro group, a C₁-C₆₀ alkyl group, a C₁-C₆₀ alkoxy group, a cyclopentyl group, a cyclohexyl group, a cyclopentenyl group, a cyclohexenyl group, a cycloheptenyl group, a phenyl group, a naphthyl group, a pentalenyl group, an indenyl group, an azulenyl group, a heptalenyl group, an acenaphthyl group, a fluorenyl group, a phenalenyl group, a phenanthrenyl group, an anthracenyl group, a fluoranthenyl group, a triphenylenyl group, a pyrenyl group, a chrysenylenyl group, a naphthacenyl group, a picenyl group, a pyridinyl group, a pyrazinyl group, a pyrimidinyl group, a pyridazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, or a carbazolyl group,
d1 and d2 are each independently an integer from 0 to 4,
n1 is an integer from 1 to 3, and
* indicates a binding site to an adjacent atom.

11. The condensed cyclic compound of claim 1, wherein
the condensed cyclic compound is represented by Formula 1A-1 or 1B-1: 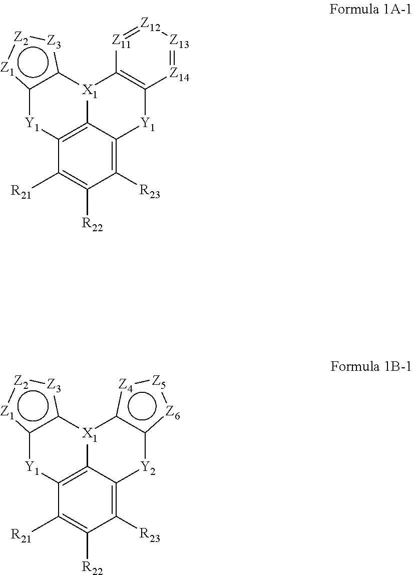
wherein, in Formula 1A-1,
Z₁₁ is C(R₁₁) or N, Z₁₂ is C(R₁₂) or N, Z₁₃ is C(R₁₃) or N, Z₁₄ is C(R₁₄) or N,
Z₄ to Z₆ are each independently O, S, N, N(R₁₁), or C(R₁₂), wherein at least two of Z₄ to Z₆ are each independently O, S, N, or N(R₁₁),
the ring including Z₄ to Z₆ is a 5-membered heteroaromatic ring,
R₁₁ to R₁₄ are each the same as described in connection with R₁ in claim 1,
R₂₁ to R₂₃ are each the same as described in connection with R₂ in claim 1, and
X₁, Y₁, Y₂, and Z₁ to Z₃ are each the same as described in claim 1.

12. The condensed cyclic compound of claim 1, wherein
the condensed cyclic compound is one of Compounds 1 to 2363:

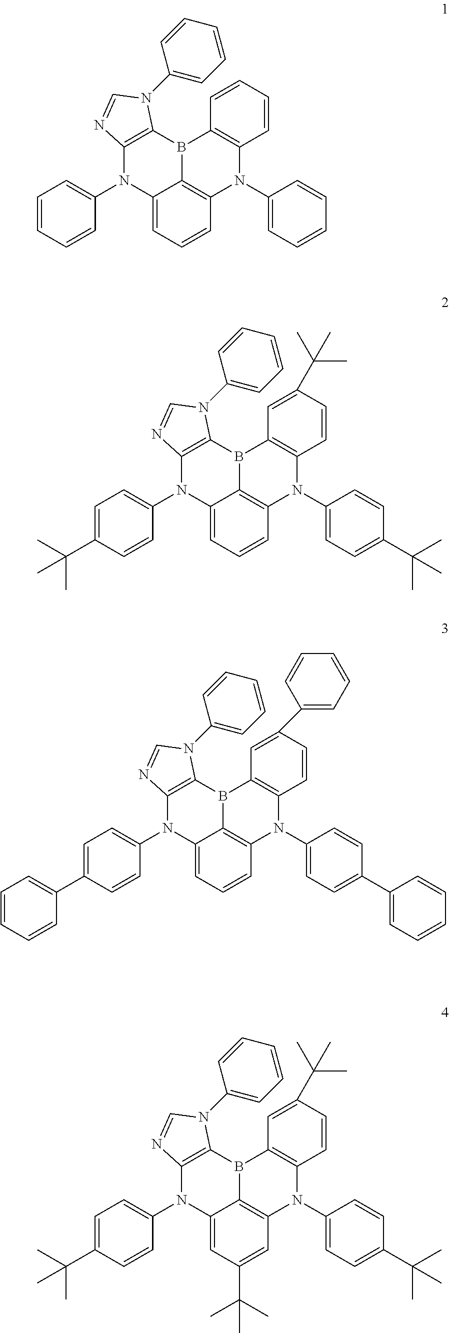

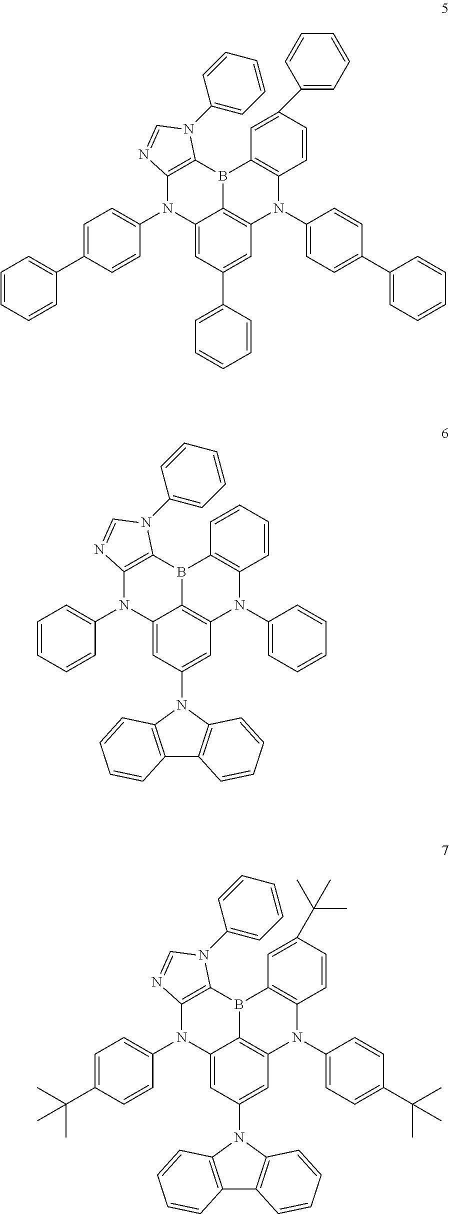 [SMILES: c1ccc(-c2ccc(N3c4ccc(-c5ccccc5)cc4B4c5c3cc(-c3ccccc3)cc5N(c3ccc(-c5ccccc5)cc3)c3ncn(-c5ccccc5)c34)cc2)cc1 ; c1ccc(N2c3ccccc3B3c4c2cc(-n2c5ccccc5c5ccccc52)cc4N(c2ccccc2)c2ncn(-c4ccccc4)c23)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2ncn(-c4ccccc4)c23)cc1]

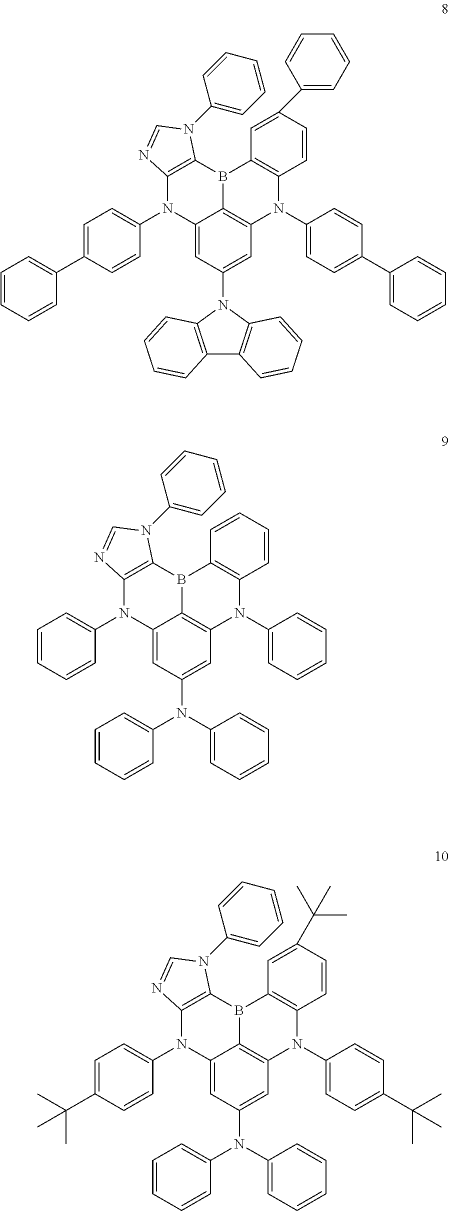 [SMILES: c1ccc(-c2ccc(N3c4ccc(-c5ccccc5)cc4B4c5c3cc(-n3c6ccccc6c6ccccc63)cc5N(c3ccc(-c5ccccc5)cc3)c3ncn(-c5ccccc5)c34)cc2)cc1 ; c1ccc(N(c2ccccc2)c2cc3c4c(c2)N(c2ccccc2)c2ncn(-c5ccccc5)c2B4c2ccccc2N3c2ccccc2)cc1 ; CC(C)(C)c1ccc(N2c3ccc(C(C)(C)C)cc3B3c4c2cc(N(c2ccccc2)c2ccccc2)cc4N(c2ccc(C(C)(C)C)cc2)c2ncn(-c4ccccc4)c23)cc1]

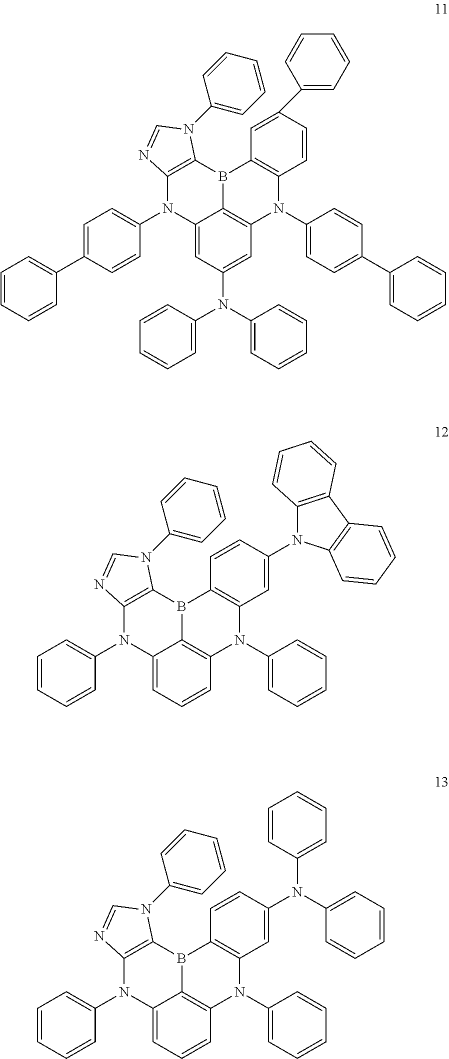 [SMILES: c1ccc(-c2ccc(N3c4ccc(-c5ccccc5)cc4B4c5c3cc(N(c3ccccc3)c3ccccc3)cc5N(c3ccc(-c5ccccc5)cc3)c3ncn(-c5ccccc5)c34)cc2)cc1 ; c1ccc(N2c3cc(-n4c5ccccc5c5ccccc54)ccc3B3c4c2cccc4N(c2ccccc2)c2ncn(-c4ccccc4)c23)cc1 ; c1ccc(N(c2ccccc2)c2ccc3c(c2)N(c2ccccc2)c2cccc4c2B3c2c(ncn2-c2ccccc2)N4c2ccccc2)cc1]

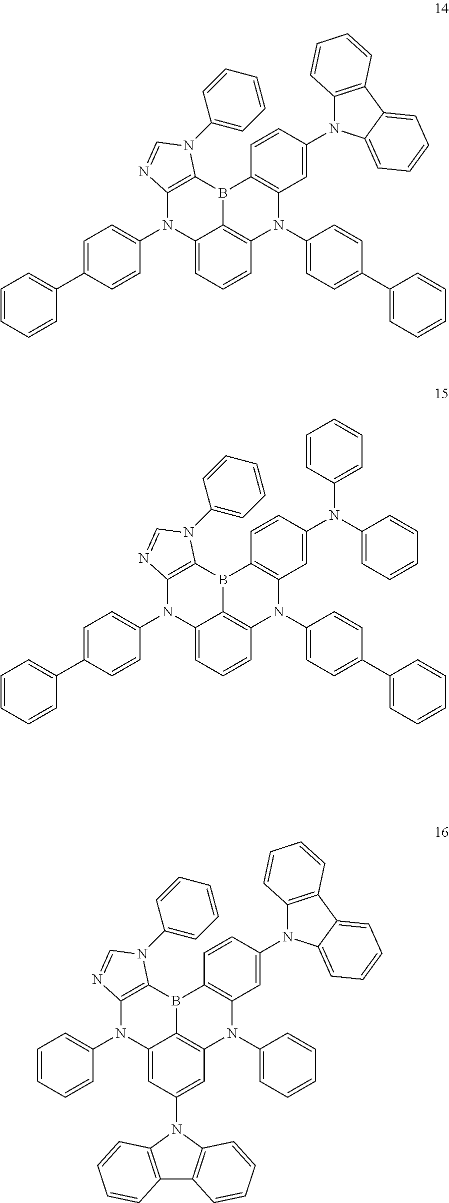 [SMILES: c1ccc(-c2ccc(N3c4cc(-n5c6ccccc6c6ccccc65)ccc4B4c5c3cccc5N(c3ccc(-c5ccccc5)cc3)c3ncn(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3ncn(-c5ccccc5)c34)cc2)cc1 ; c1ccc(N2c3cc(-n4c5ccccc5c5ccccc54)ccc3B3c4c2cc(-n2c5ccccc5c5ccccc52)cc4N(c2ccccc2)c2ncn(-c4ccccc4)c23)cc1]

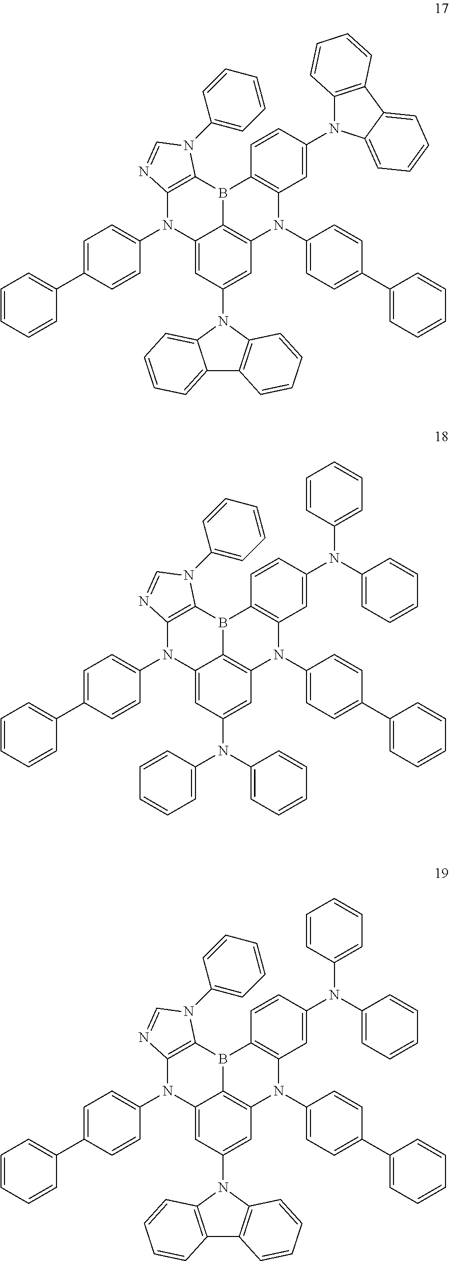 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3ncn(-c5ccccc5)c34)cc2)cc1 ; c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3ncn(-c5ccccc5)c34)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3ncn(-c5ccccc5)c34)cc2)cc1]

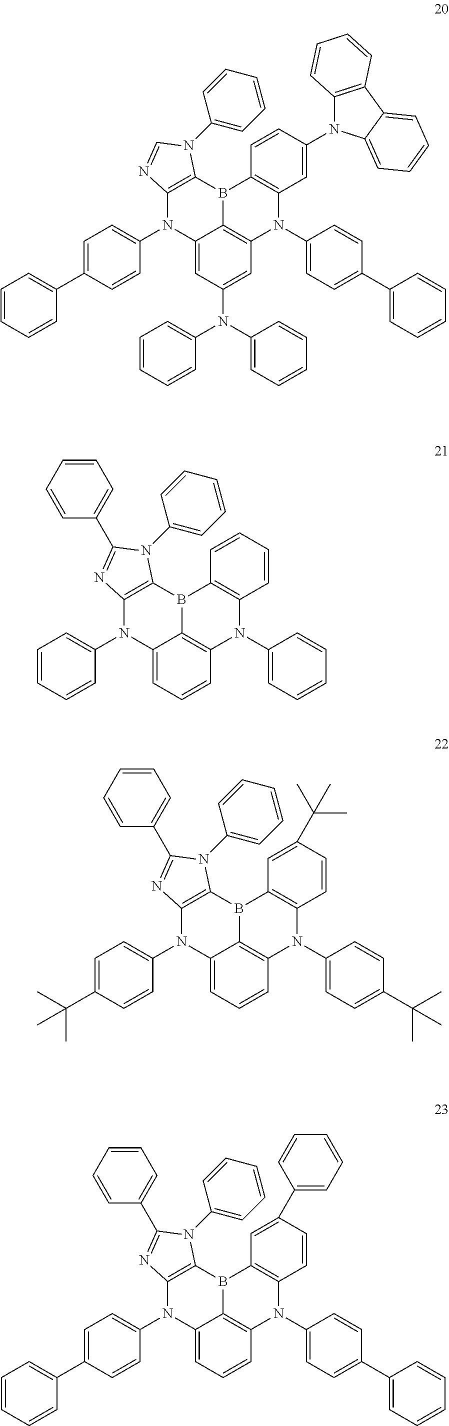 [SMILES: c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3ncn(-c5ccccc5)c34)cc2)cc1 ; c1ccc(-c2nc3c(n2-c2ccccc2)B2c4ccccc4N(c4ccccc4)c4cccc(c42)N3c2ccccc2)cc1 ; CC(C)(C)c1ccc(N2c3ccc(C(C)(C)C)cc3B3c4c2cccc4N(c2ccc(C(C)(C)C)cc2)c2nc(-c4ccccc4)n(-c4ccccc4)c23)cc1 ; c1ccc(-c2ccc(N3c4ccc(-c5ccccc5)cc4B4c5c3cccc5N(c3ccc(-c5ccccc5)cc3)c3nc(-c5ccccc5)n(-c5ccccc5)c34)cc2)cc1]

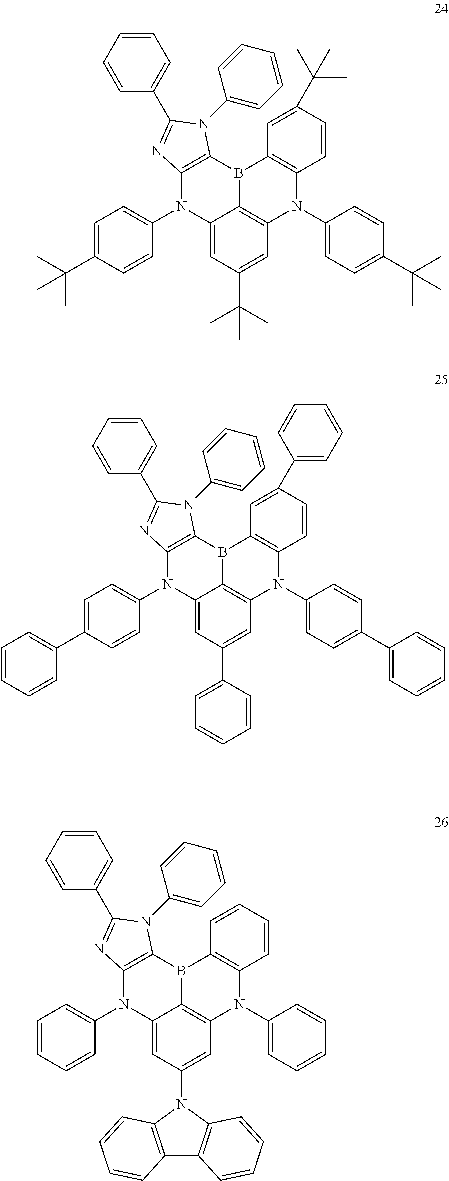 [SMILES: CC(C)(C)c1ccc(N2c3ccc(C(C)(C)C)cc3B3c4c2cc(C(C)(C)C)cc4N(c2ccc(C(C)(C)C)cc2)c2nc(-c4ccccc4)n(-c4ccccc4)c23)cc1 ; c1ccc(-c2ccc(N3c4ccc(-c5ccccc5)cc4B4c5c3cc(-c3ccccc3)cc5N(c3ccc(-c5ccccc5)cc3)c3nc(-c5ccccc5)n(-c5ccccc5)c34)cc2)cc1 ; c1ccc(-c2nc3c(n2-c2ccccc2)B2c4ccccc4N(c4ccccc4)c4cc(-n5c6ccccc6c6ccccc65)cc(c42)N3c2ccccc2)cc1]

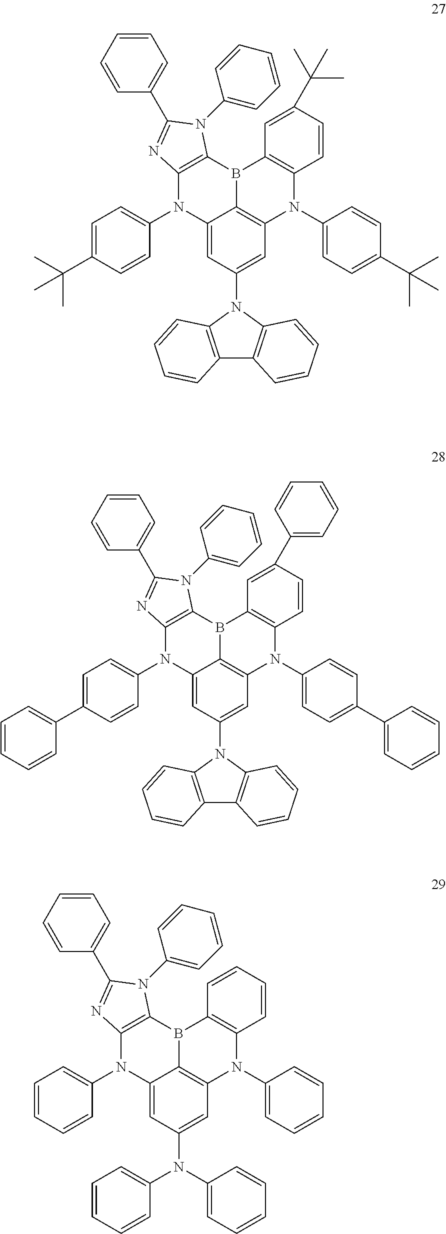 [SMILES: CC(C)(C)c1ccc(N2c3ccc(C(C)(C)C)cc3B3c4c2cc(-n2c5ccccc5c5ccccc52)cc4N(c2ccc(C(C)(C)C)cc2)c2nc(-c4ccccc4)n(-c4ccccc4)c23)cc1 ; c1ccc(-c2ccc(N3c4ccc(-c5ccccc5)cc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; c1ccc(-c2nc3c(n2-c2ccccc2)B2c4ccccc4N(c4ccccc4)c4cc(N(c5ccccc5)c5ccccc5)cc(c42)N3c2ccccc2)cc1]

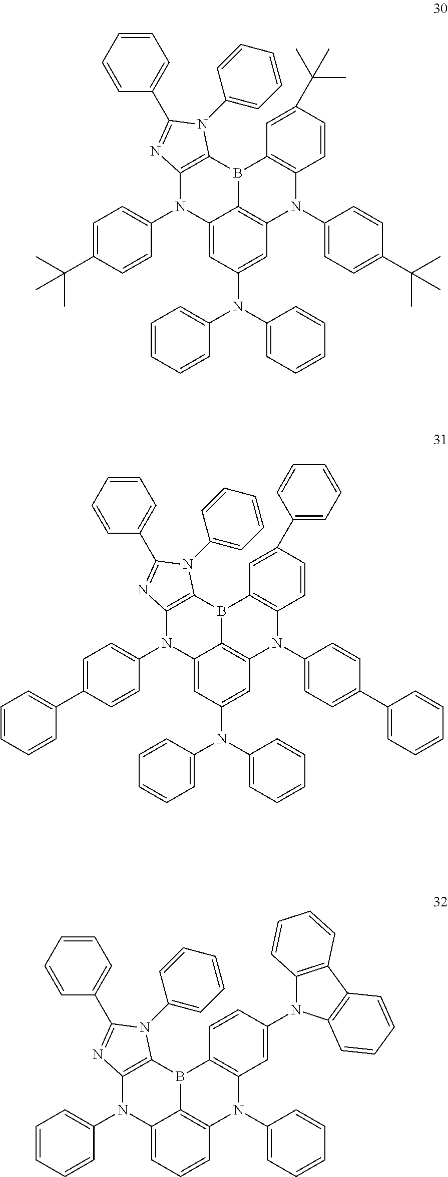 [SMILES: CC(C)(C)c1ccc(N2c3ccc(C(C)(C)C)cc3B3c4c2cc(N(c2ccccc2)c2ccccc2)cc4N(c2ccc(C(C)(C)C)cc2)c2nc(-c4ccccc4)n(-c4ccccc4)c23)cc1 ; c1ccc(-c2ccc(N3c4ccc(-c5ccccc5)cc4B4c5c3cc(N(c3ccccc3)c3ccccc3)cc5N(c3ccc(-c5ccccc5)cc3)c3nc(-c5ccccc5)n(-c5ccccc5)c34)cc2)cc1 ; c1ccc(-c2nc3c(n2-c2ccccc2)B2c4ccc(-n5c6ccccc6c6ccccc65)cc4N(c4ccccc4)c4cccc(c42)N3c2ccccc2)cc1]

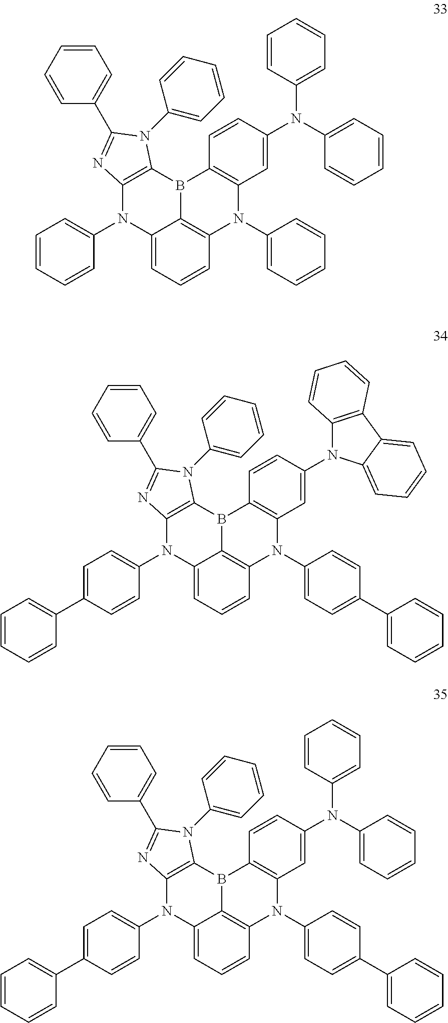 [SMILES: c1ccc(-c2nc3c(n2-c2ccccc2)B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5c3cccc5N(c3ccc(-c5ccccc5)cc3)c3nc(-c5ccccc5)n(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3nc(-c5ccccc5)n(-c5ccccc5)c34)cc2)cc1]

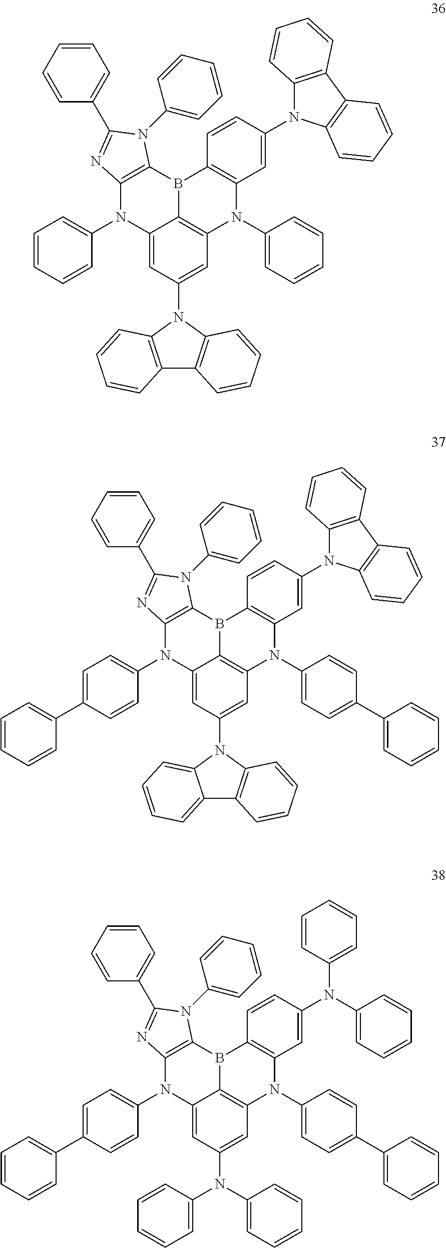 [SMILES: c1ccc(-c2nc3c(n2-c2ccccc2)B2c4ccc(-n5c6ccccc6c6ccccc65)cc4N(c4ccccc4)c4cc(-n5c6ccccc6c6ccccc65)cc(c42)N3c2ccccc2)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3nc(-c5ccccc5)n(-c5ccccc5)c34)cc2)cc1]

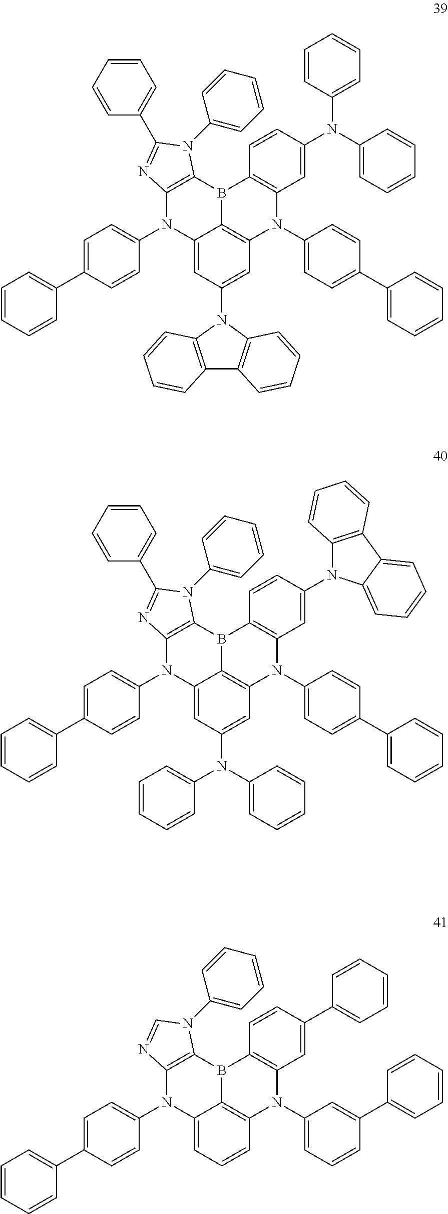 [SMILES: c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3nc(-c5ccccc5)n(-c5ccccc5)c34)cc2)cc1 ; c1ccc(-c2ccc(N3c4cccc5c4B(c4ccc(-c6ccccc6)cc4N5c4cccc(-c5ccccc5)c4)c4c3ncn4-c3ccccc3)cc2)cc1]

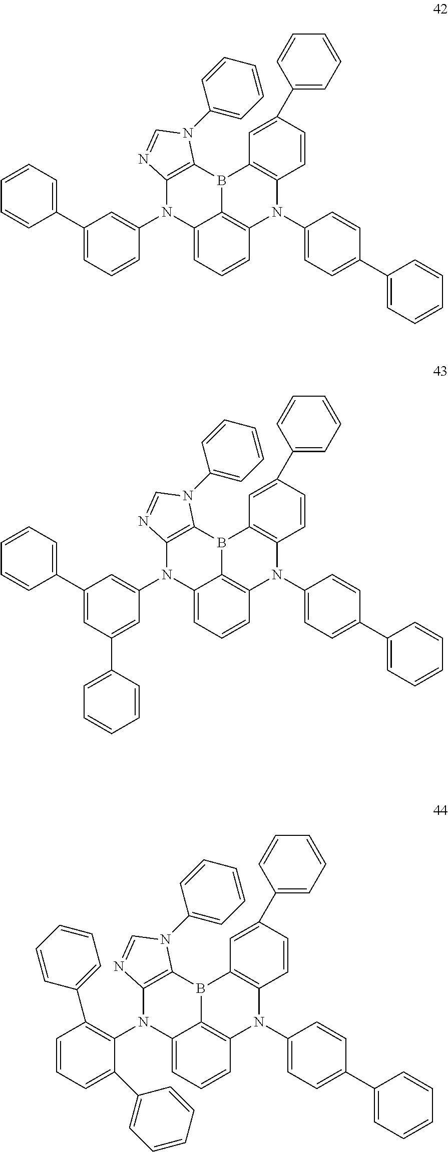 [SMILES: c1ccc(-c2ccc(N3c4ccc(-c5ccccc5)cc4B4c5c3cccc5N(c3cccc(-c5ccccc5)c3)c3ncn(-c5ccccc5)c34)cc2)cc1 ; c1ccc(-c2ccc(N3c4ccc(-c5ccccc5)cc4B4c5c3cccc5N(c3cc(-c5ccccc5)cc(-c5ccccc5)c3)c3ncn(-c5ccccc5)c34)cc2)cc1 ; c1ccc(-c2ccc(N3c4ccc(-c5ccccc5)cc4B4c5c3cccc5N(c3c(-c5ccccc5)cccc3-c3ccccc3)c3ncn(-c5ccccc5)c34)cc2)cc1]

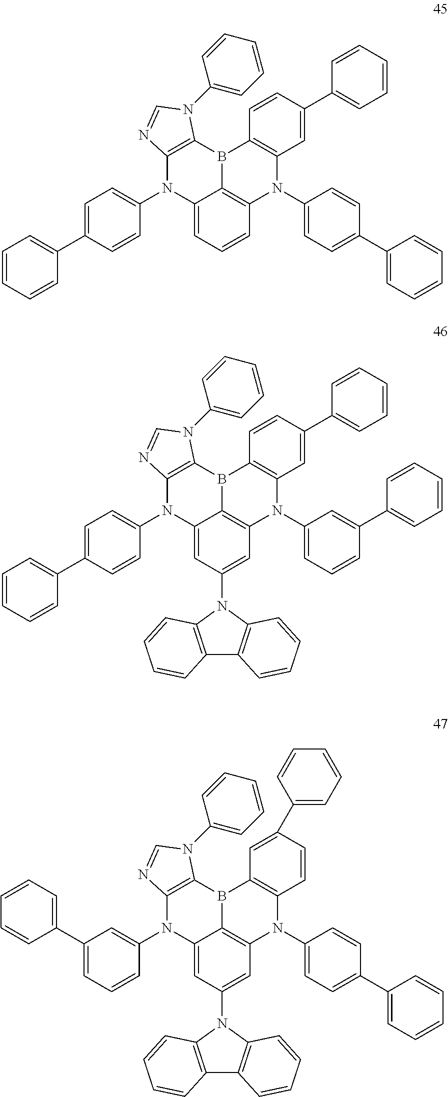 [SMILES: c1ccc(-c2ccc(N3c4cc(-c5ccccc5)ccc4B4c5c3cccc5N(c3ccc(-c5ccccc5)cc3)c3ncn(-c5ccccc5)c34)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c3ncn4-c3ccccc3)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3ncn(-c5ccccc5)c34)cc2)cc1]

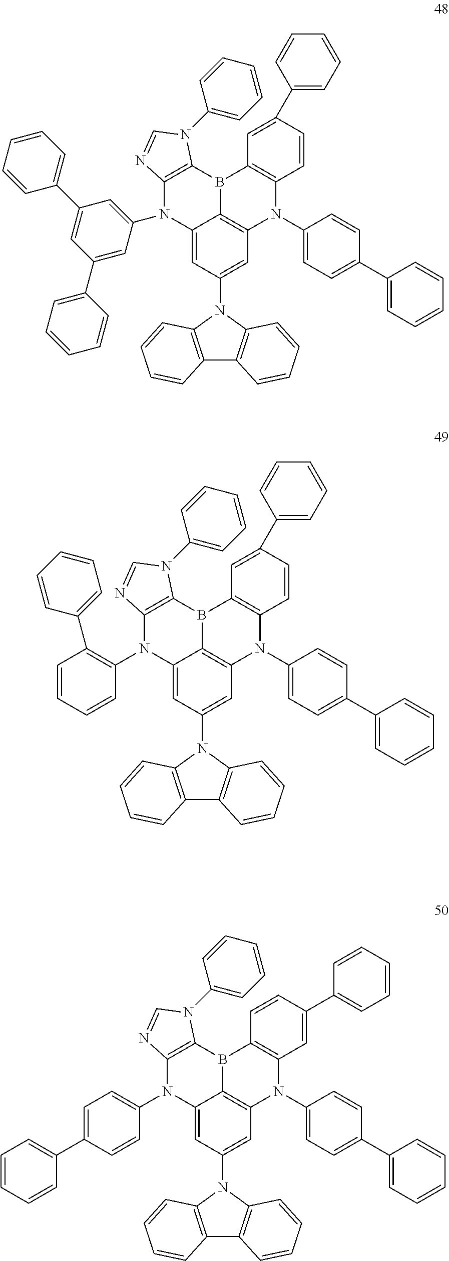 [SMILES: c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3ncn(-c5ccccc5)c34)cc2)cc1 ; c1ccc(-c2ccc(N3c4ccc(-c5ccccc5)cc4B4c5c3cc(-n3c6ccccc6c6ccccc63)cc5N(c3ccccc3-c3ccccc3)c3ncn(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3ncn(-c5ccccc5)c34)cc2)cc1]

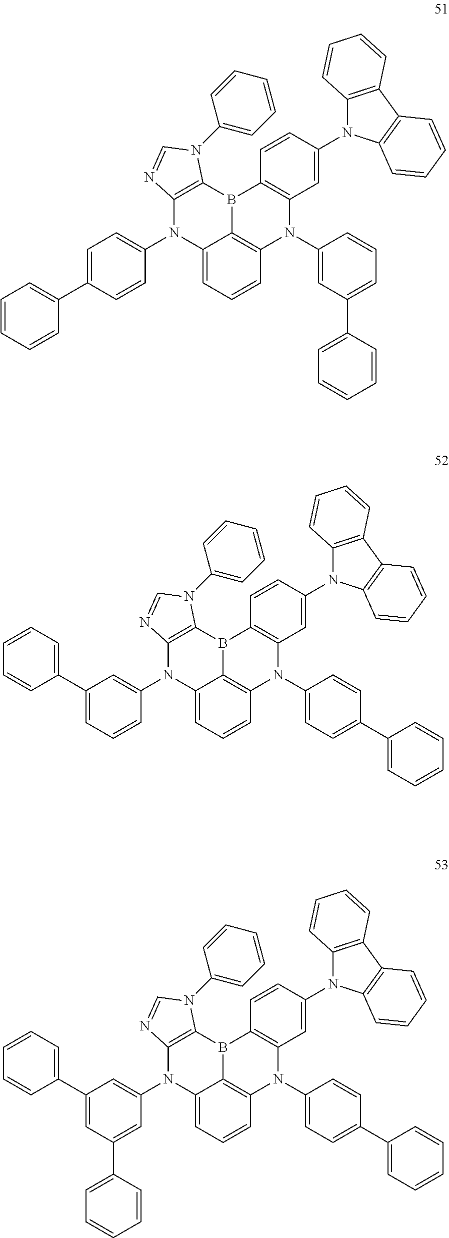 [SMILES: c1ccc(-c2ccc(N3c4cccc5c4B(c4ccc(-n6c7ccccc7c7ccccc76)cc4N5c4cccc(-c5ccccc5)c4)c4c3ncn4-c3ccccc3)cc2)cc1 ; c1ccc(-c2ccc(N3c4cc(-n5c6ccccc6c6ccccc65)ccc4B4c5c3cccc5N(c3cccc(-c5ccccc5)c3)c3ncn(-c5ccccc5)c34)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3ncn(-c5ccccc5)c34)cc2)cc1]

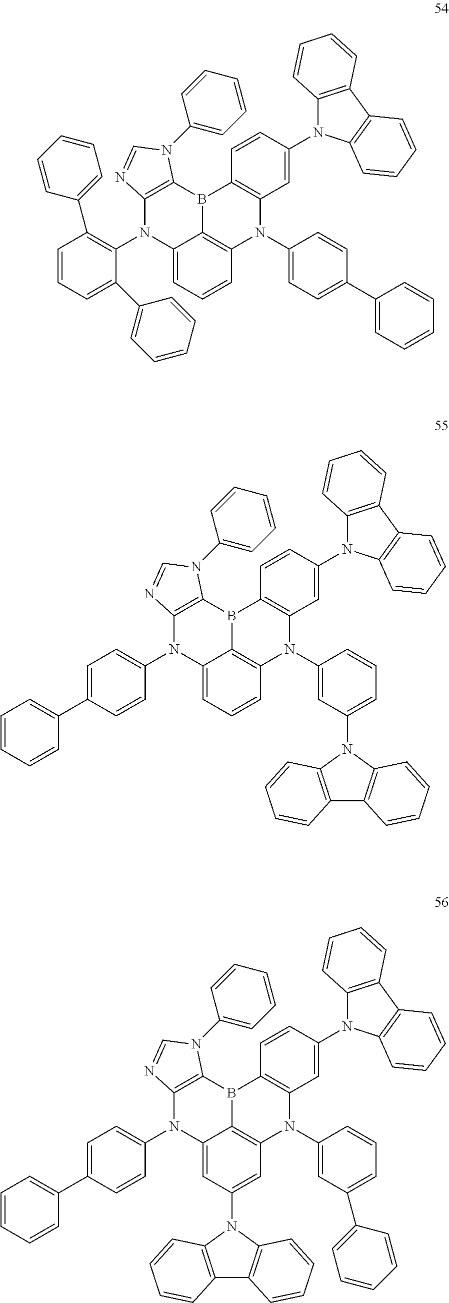 [SMILES: c1ccc(-c2ccc(N3c4cc(-n5c6ccccc6c6ccccc65)ccc4B4c5c3cccc5N(c3c(-c5ccccc5)cccc3-c3ccccc3)c3ncn(-c5ccccc5)c34)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4c3ncn4-c3ccccc3)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3ncn4-c3ccccc3)cc2)cc1]

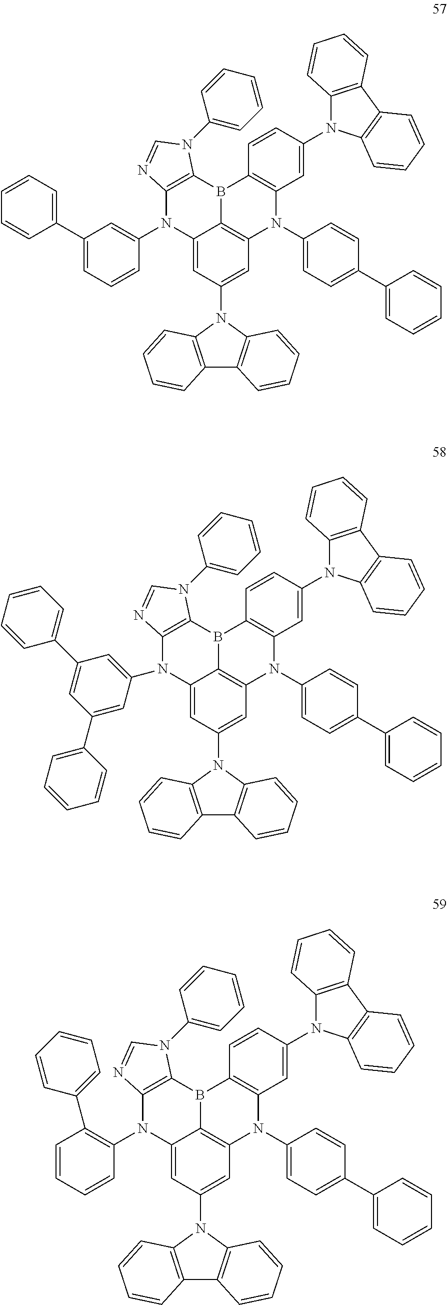 [SMILES: c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3ncn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3ncn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3ncn(-c5ccccc5)c34)cc2)cc1]

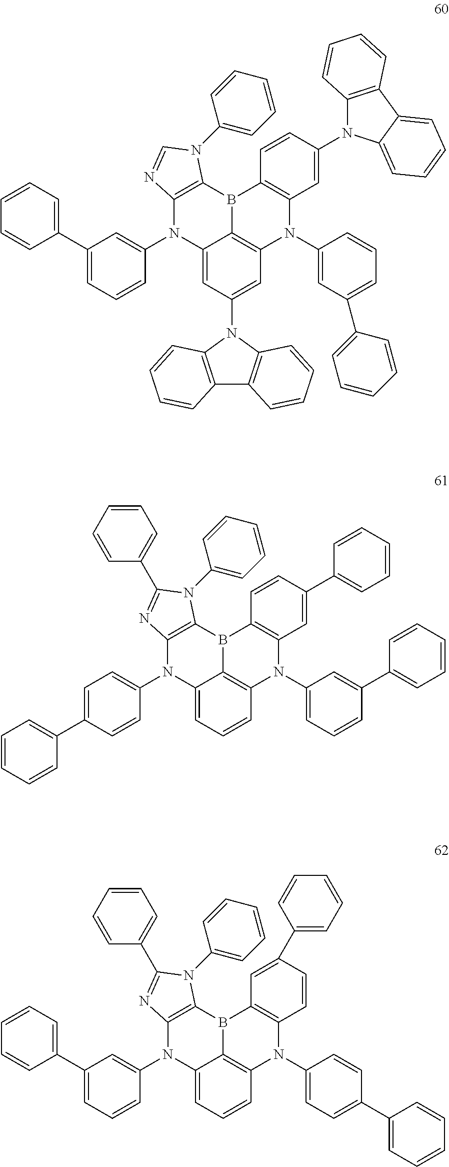 [SMILES: c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3ncn(-c5ccccc5)c34)c2)cc1 ; c1ccc(-c2ccc(N3c4cccc5c4B(c4ccc(-c6ccccc6)cc4N5c4cccc(-c5ccccc5)c4)c4c3nc(-c3ccccc3)n4-c3ccccc3)cc2)cc1 ; c1ccc(-c2ccc(N3c4ccc(-c5ccccc5)cc4B4c5c3cccc5N(c3cccc(-c5ccccc5)c3)c3nc(-c5ccccc5)n(-c5ccccc5)c34)cc2)cc1]

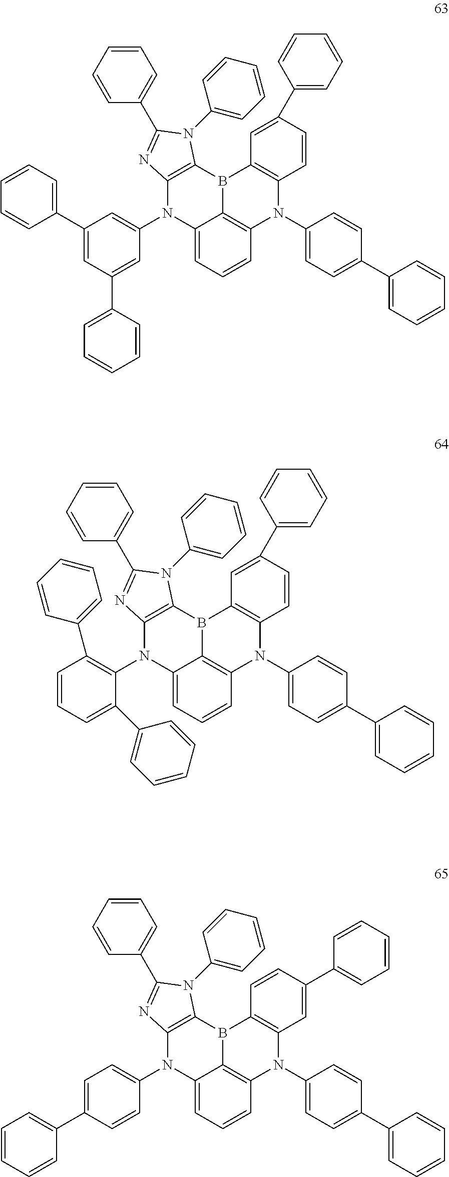 [SMILES: c1ccc(-c2ccc(N3c4ccc(-c5ccccc5)cc4B4c5c3cccc5N(c3cc(-c5ccccc5)cc(-c5ccccc5)c3)c3nc(-c5ccccc5)n(-c5ccccc5)c34)cc2)cc1 ; c1ccc(-c2ccc(N3c4ccc(-c5ccccc5)cc4B4c5c3cccc5N(c3c(-c5ccccc5)cccc3-c3ccccc3)c3nc(-c5ccccc5)n(-c5ccccc5)c34)cc2)cc1 ; c1ccc(-c2ccc(N3c4cc(-c5ccccc5)ccc4B4c5c3cccc5N(c3ccc(-c5ccccc5)cc3)c3nc(-c5ccccc5)n(-c5ccccc5)c34)cc2)cc1]

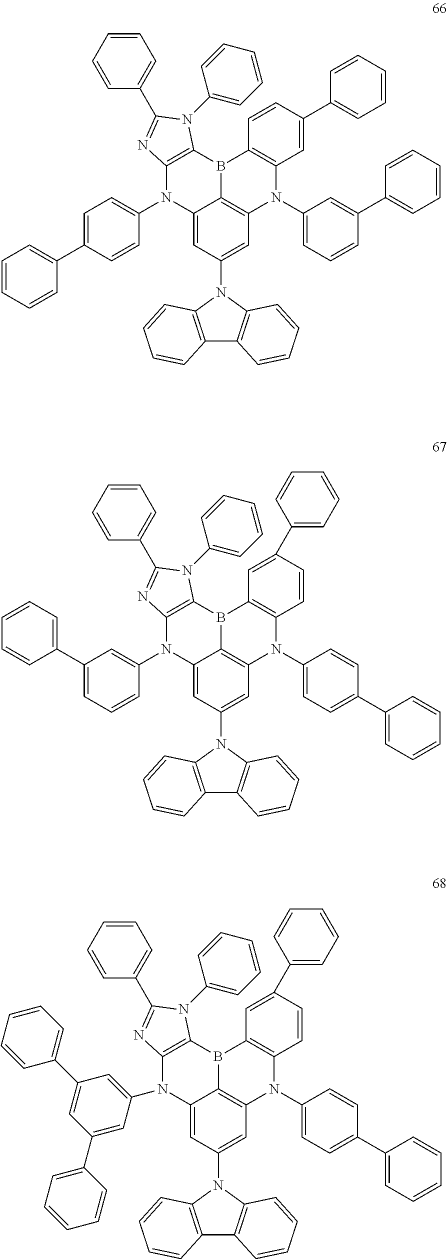 [SMILES: c1ccc(-c2ccc(N3c4cc(-n5c6ccccc6c6ccccc65)cc5c4B(c4ccc(-c6ccccc6)cc4N5c4cccc(-c5ccccc5)c4)c4c3nc(-c3ccccc3)n4-c3ccccc3)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3nc(-c5ccccc5)n(-c5ccccc5)c34)cc2)cc1 ; c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3nc(-c5ccccc5)n(-c5ccccc5)c34)cc2)cc1]

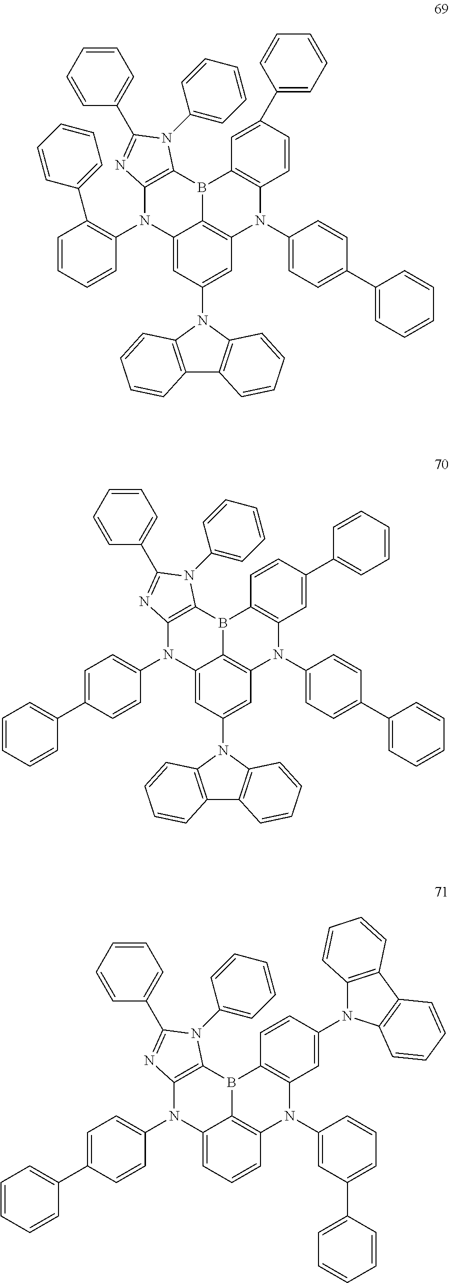 [SMILES: c1ccc(-c2ccc(N3c4ccc(-c5ccccc5)cc4B4c5c3cc(-n3c6ccccc6c6ccccc63)cc5N(c3ccccc3-c3ccccc3)c3nc(-c5ccccc5)n(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1 ; c1ccc(-c2ccc(N3c4cccc5c4B(c4ccc(-n6c7ccccc7c7ccccc76)cc4N5c4cccc(-c5ccccc5)c4)c4c3nc(-c3ccccc3)n4-c3ccccc3)cc2)cc1]

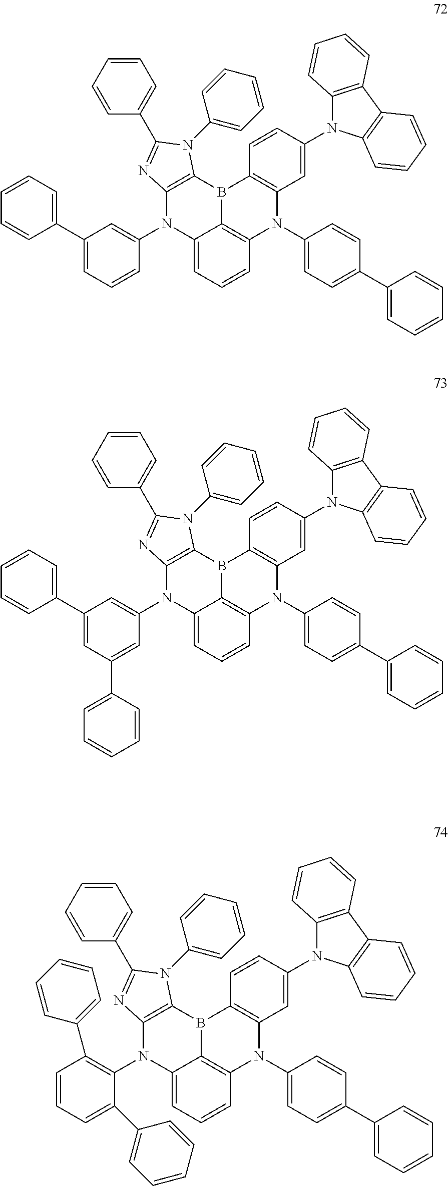 [SMILES: c1ccc(-c2ccc(N3c4cc(-n5c6ccccc6c6ccccc65)ccc4B4c5c3cccc5N(c3cccc(-c5ccccc5)c3)c3nc(-c5ccccc5)n(-c5ccccc5)c34)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3nc(-c5ccccc5)n(-c5ccccc5)c34)cc2)cc1 ; c1ccc(-c2ccc(N3c4cc(-n5c6ccccc6c6ccccc65)ccc4B4c5c3cccc5N(c3c(-c5ccccc5)cccc3-c3ccccc3)c3nc(-c5ccccc5)n(-c5ccccc5)c34)cc2)cc1]

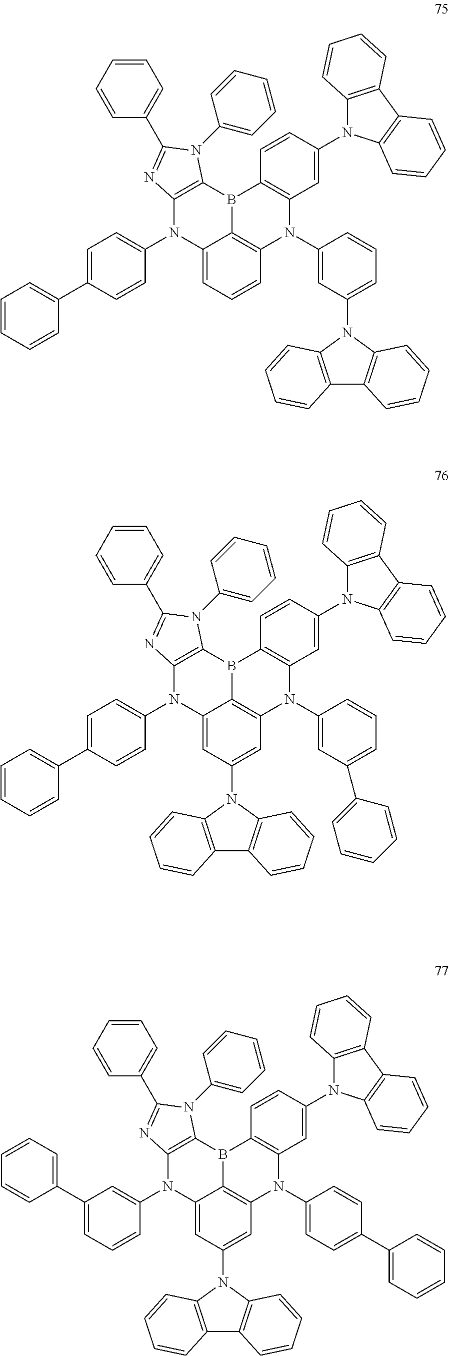 [SMILES: c1ccc(-c2ccc(N3c4cccc5c4B(c4ccc(-n6c7ccccc7c7ccccc76)cc4N5c4cccc(-n5c6ccccc6c6ccccc65)c4)c4c3nc(-c3ccccc3)n4-c3ccccc3)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3nc(-c3ccccc3)n4-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3nc(-c5ccccc5)n(-c5ccccc5)c34)cc2)cc1]

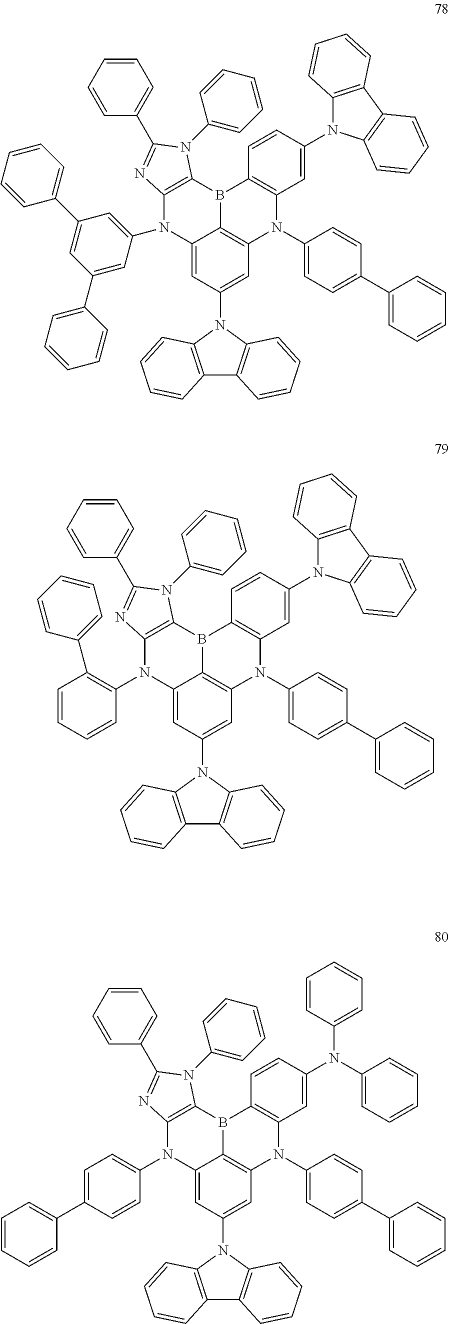 [SMILES: c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3nc(-c5ccccc5)n(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3nc(-c5ccccc5)n(-c5ccccc5)c34)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3nc(-c5ccccc5)n(-c5ccccc5)c34)cc2)cc1]

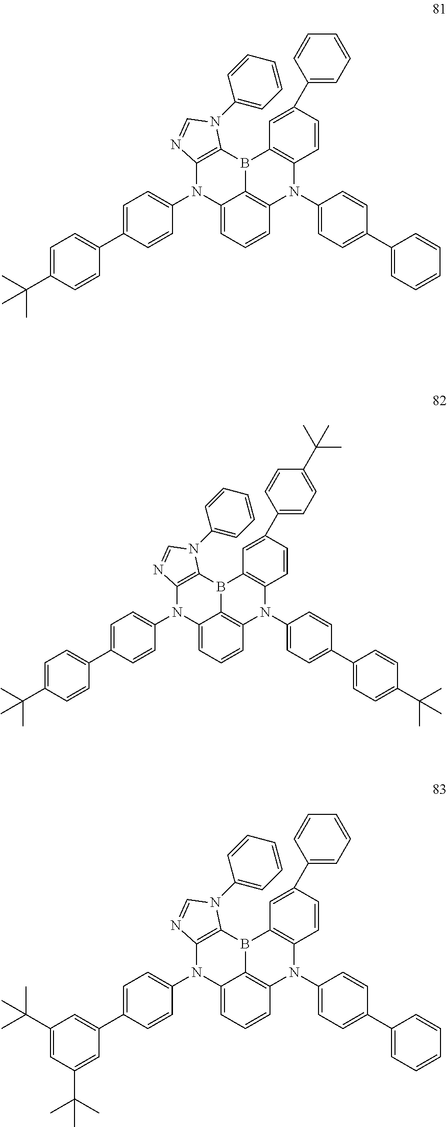

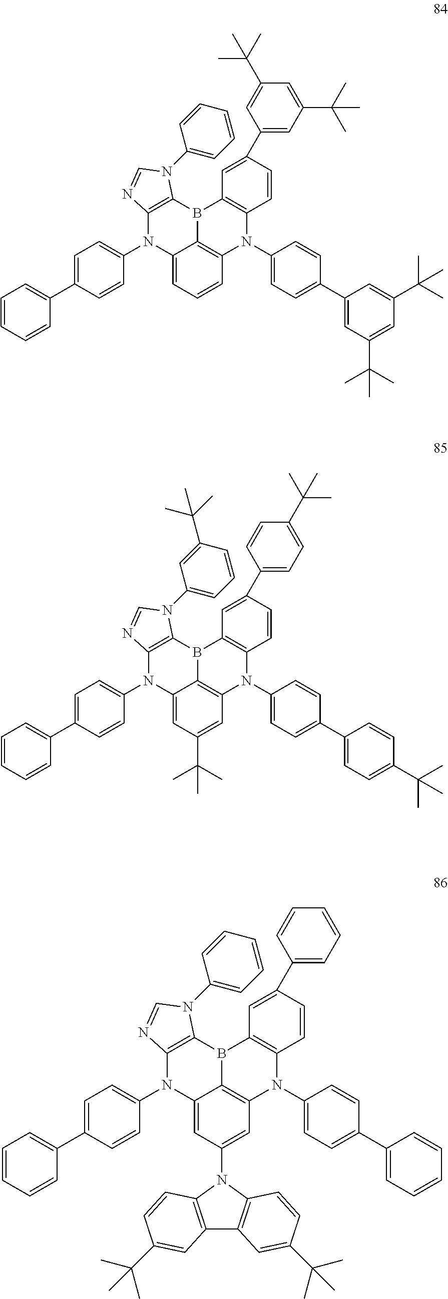 [SMILES: CC(C)(C)c1cc(-c2ccc(N3c4ccc(-c5cc(C(C)(C)C)cc(C(C)(C)C)c5)cc4B4c5c3cccc5N(c3ccc(-c5ccccc5)cc3)c3ncn(-c5ccccc5)c34)cc2)cc(C(C)(C)C)c1 ; CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3ncn(-c5cccc(C(C)(C)C)c5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1ncn(-c4ccccc4)c1B3c1cc(-c3ccccc3)ccc1N2c1ccc(-c2ccccc2)cc1]

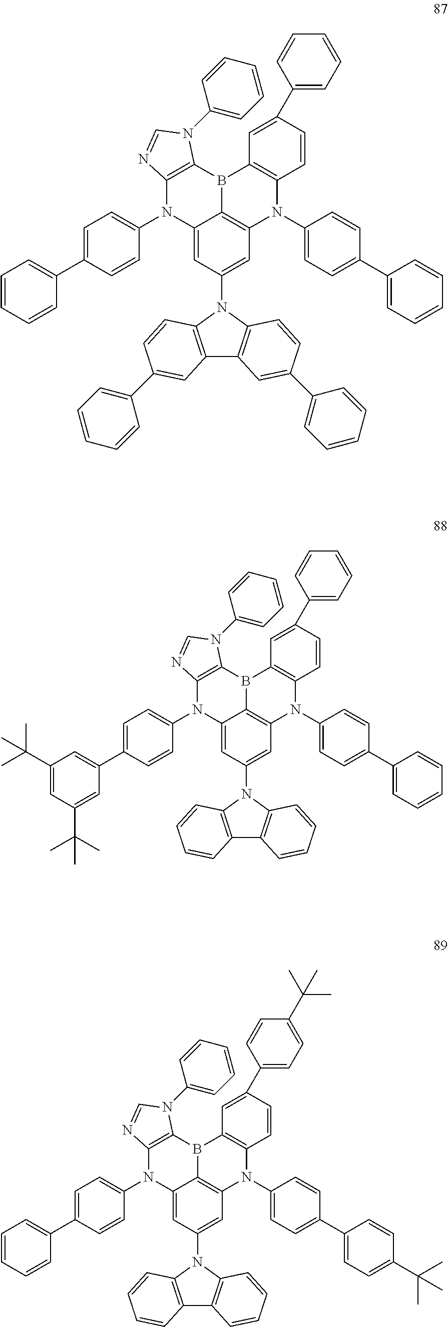 [SMILES: c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3ncn(-c5ccccc5)c34)cc2)cc1 ; CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4c3ncn4-c3ccccc3)cc2)cc(C(C)(C)C)c1 ; CC(C)(C)c1ccc(-c2ccc(N3c4ccc(-c5ccc(C(C)(C)C)cc5)cc4B4c5c3cc(-n3c6ccccc6c6ccccc63)cc5N(c3ccc(-c5ccccc5)cc3)c3ncn(-c5ccccc5)c34)cc2)cc1]

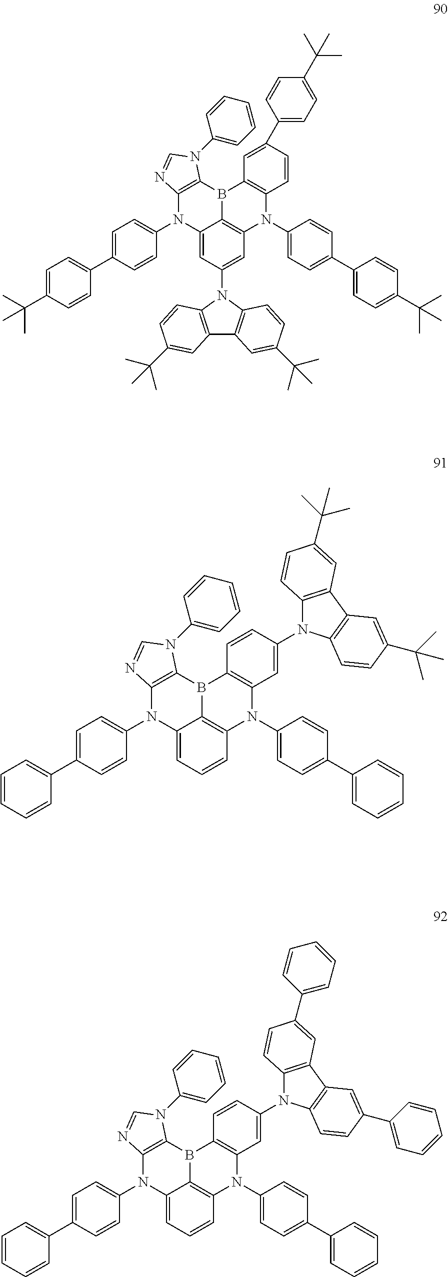 [SMILES: CC(C)(C)c1ccc(-c2ccc(N3c4ccc(-c5ccc(C(C)(C)C)cc5)cc4B4c5c3cc(-n3c6ccc(C(C)(C)C)cc6c6cc(C(C)(C)C)ccc63)cc5N(c3ccc(-c5ccc(C(C)(C)C)cc5)cc3)c3ncn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(ncn1-c1ccccc1)N3c1ccc(-c2ccccc2)cc1 ; c1ccc(-c2ccc(N3c4cc(-n5c6ccc(-c7ccccc7)cc6c6cc(-c7ccccc7)ccc65)ccc4B4c5c3cccc5N(c3ccc(-c5ccccc5)cc3)c3ncn(-c5ccccc5)c34)cc2)cc1]

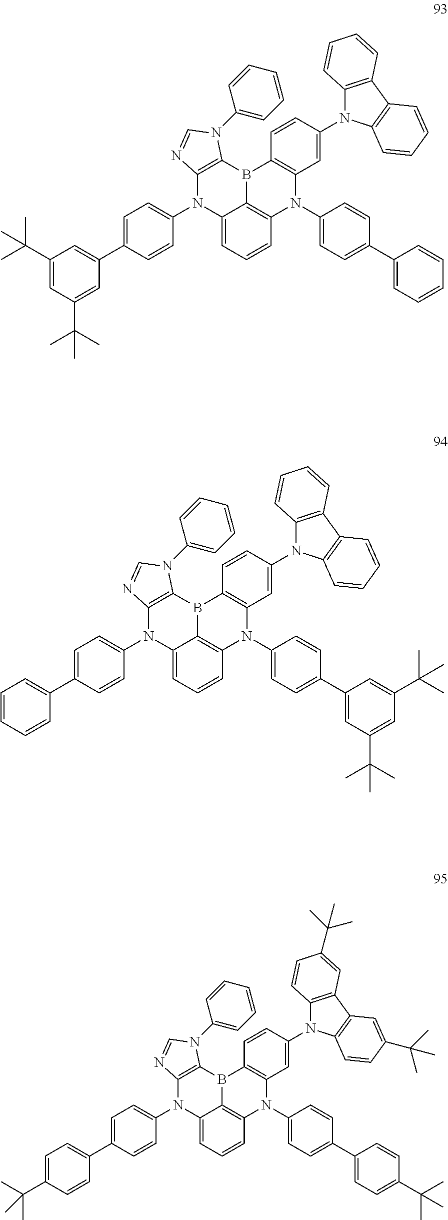

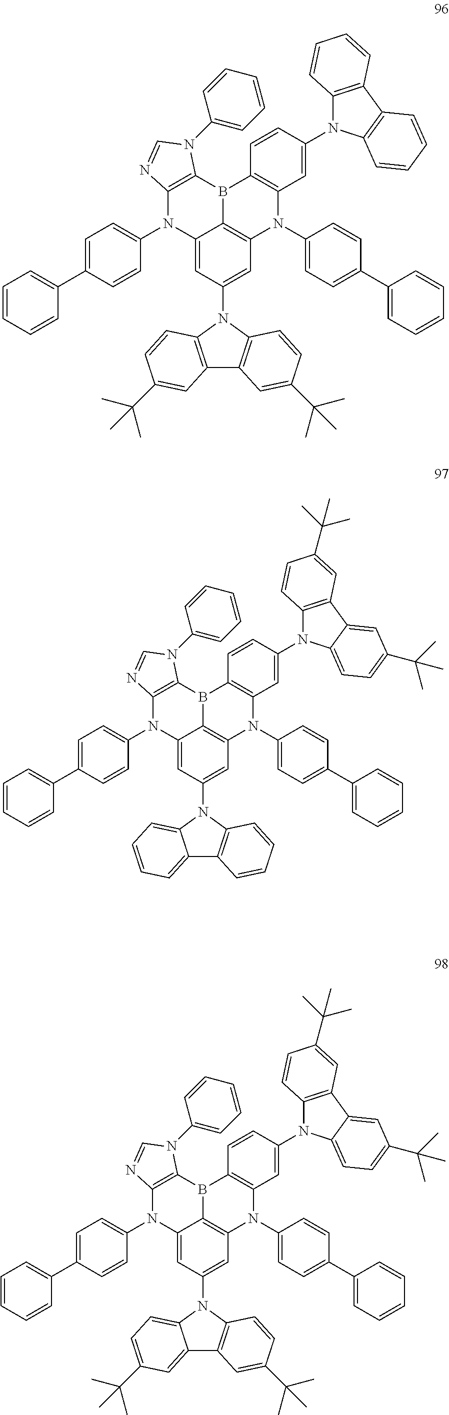 [SMILES: CC(C)(C)c1ccc2c(c1)c1cc(C(C)(C)C)ccc1n2-c1cc2c3c(c1)N(c1ccc(-c4ccccc4)cc1)c1ncn(-c4ccccc4)c1B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(ncn1-c1ccccc1)N3c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccc(C(C)(C)C)cc4c4cc(C(C)(C)C)ccc43)cc3c1B2c1c(ncn1-c1ccccc1)N3c1ccc(-c2ccccc2)cc1]

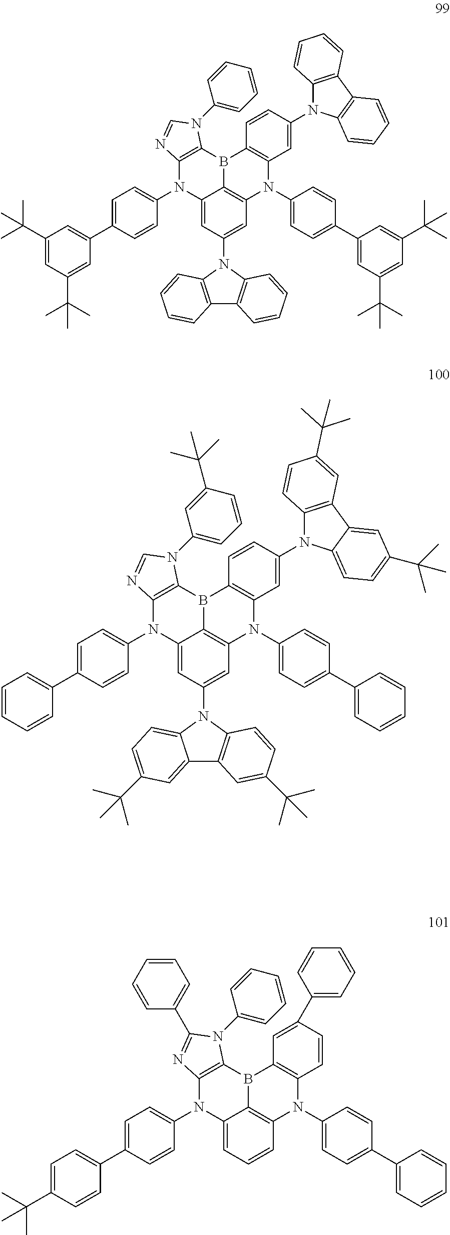

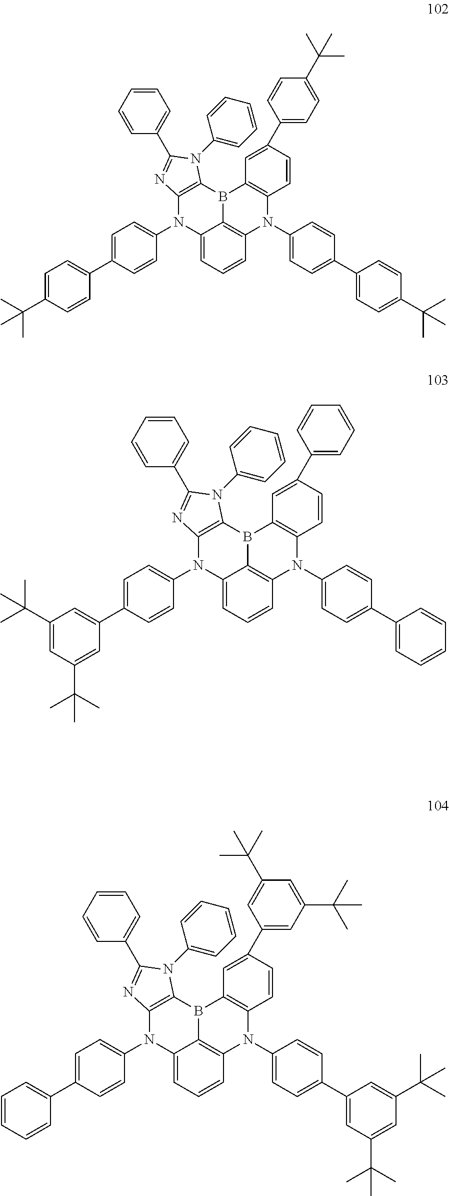

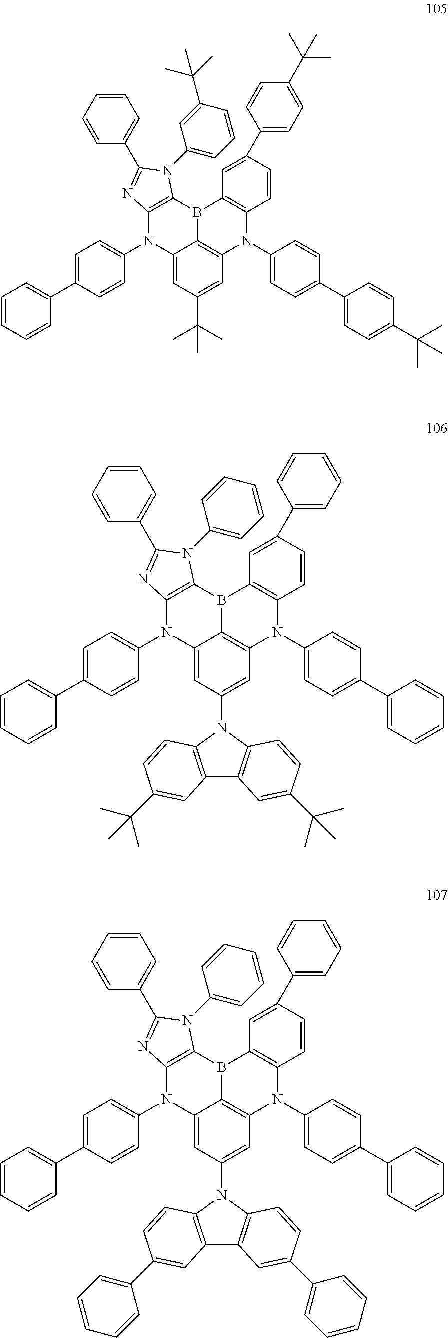 [SMILES: CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3nc(-c5ccccc5)n(-c5cccc(C(C)(C)C)c5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1nc(-c4ccccc4)n(-c4ccccc4)c1B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3nc(-c5ccccc5)n(-c5ccccc5)c34)cc2)cc1]

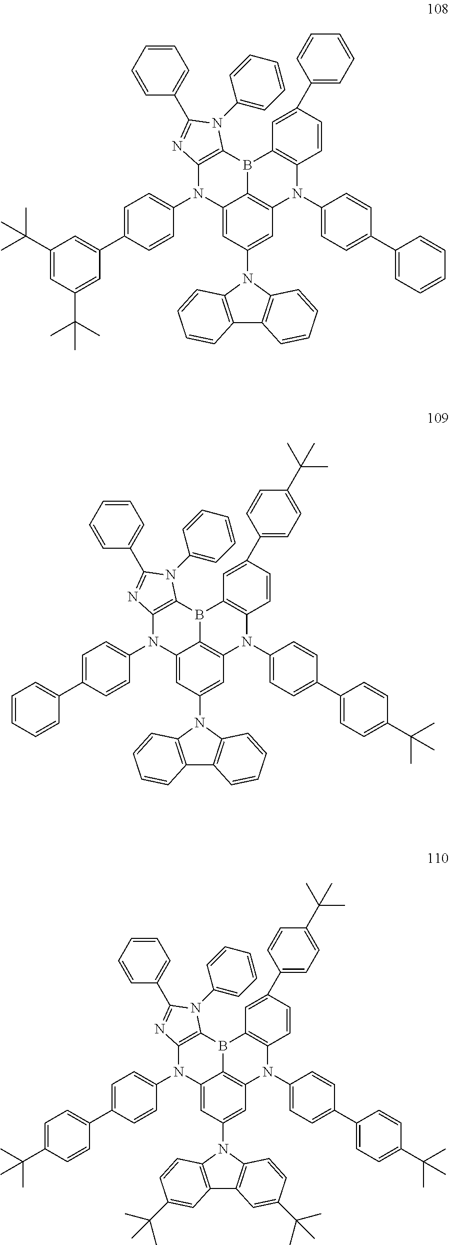

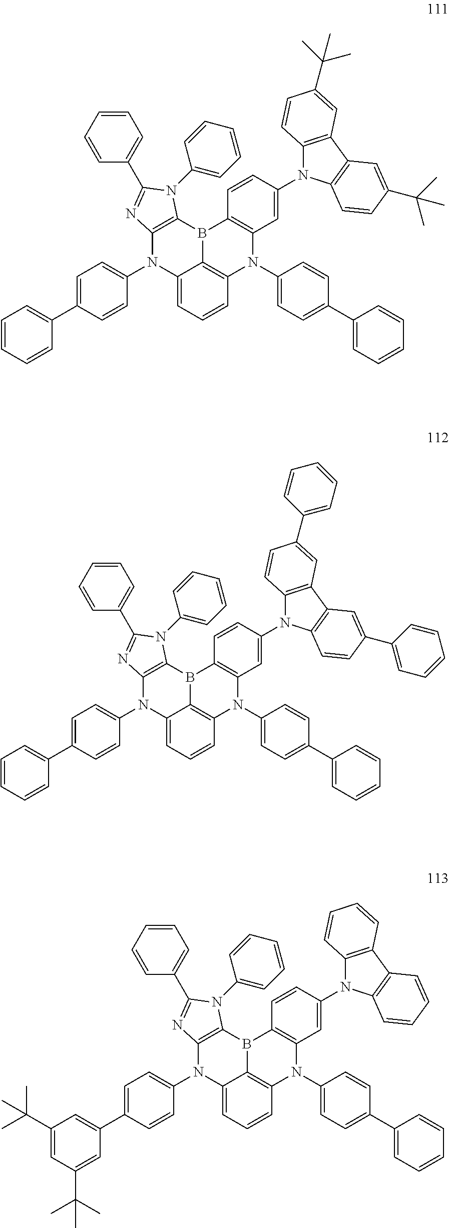

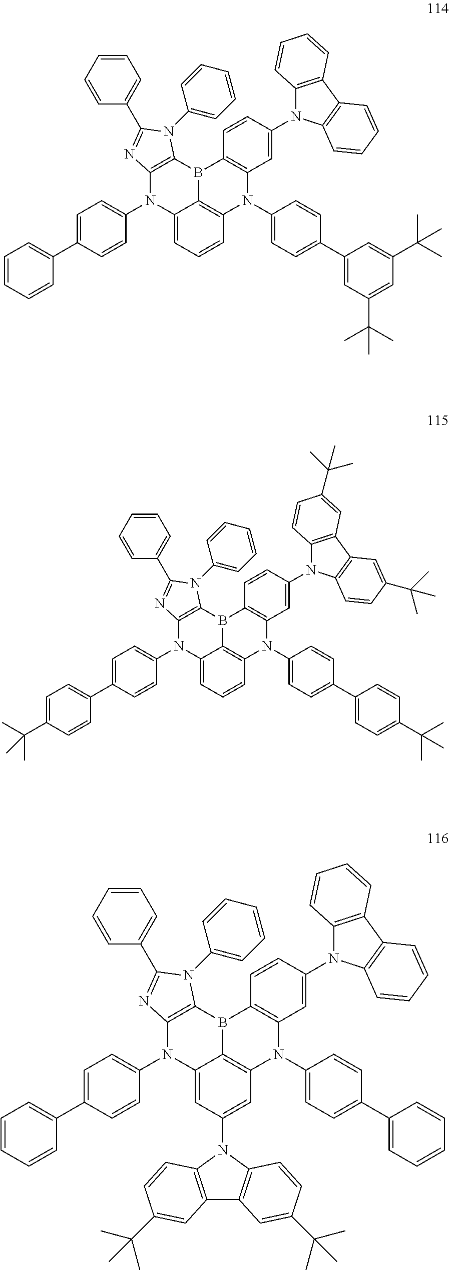

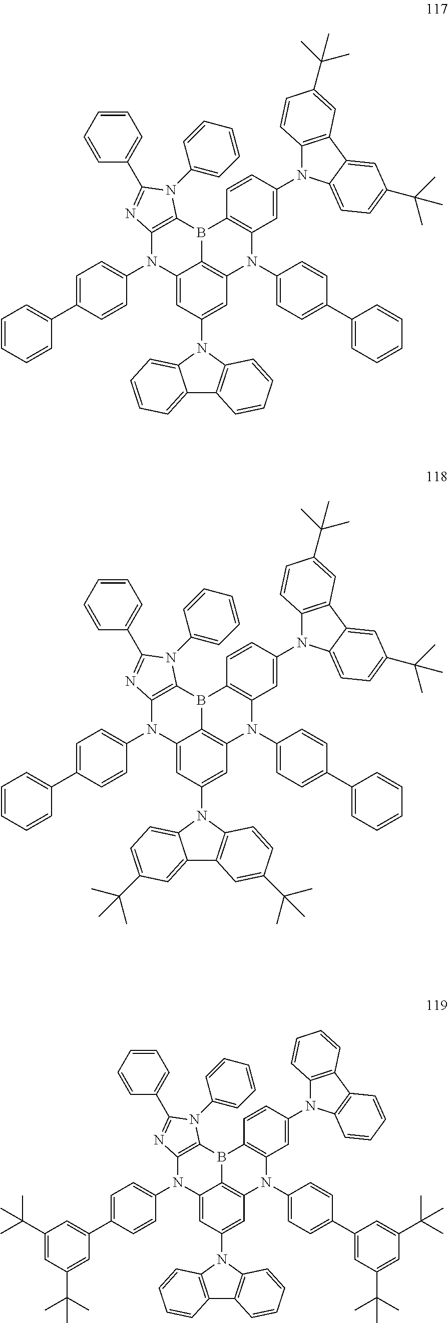

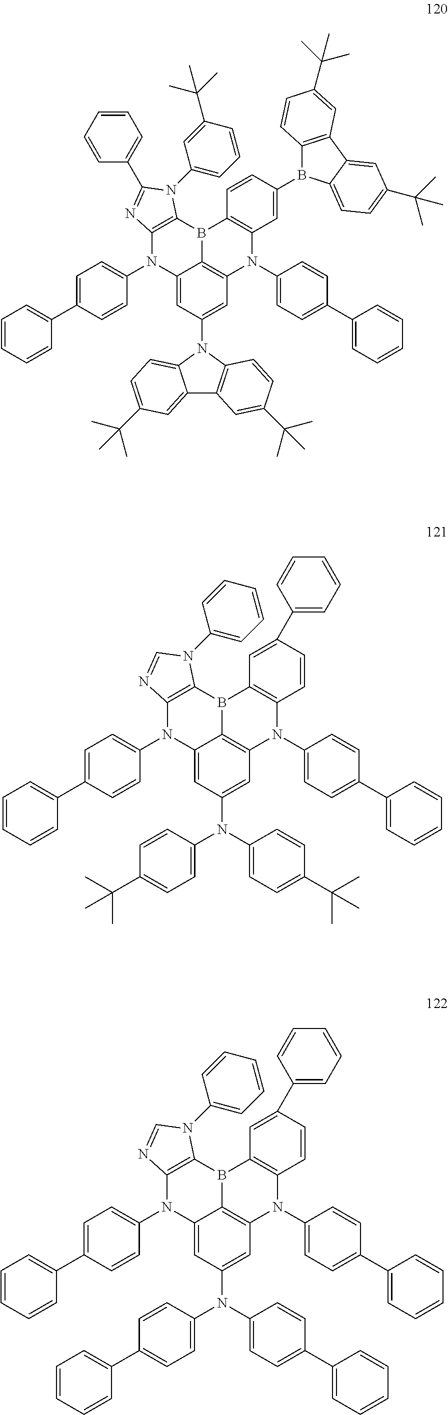 [SMILES: CC(C)(C)c1cccc(-n2c(-c3ccccc3)nc3c2B2c4ccc(B5c6ccc(C(C)(C)C)cc6-c6cc(C(C)(C)C)ccc65)cc4N(c4ccc(-c5ccccc5)cc4)c4cc(-n5c6ccc(C(C)(C)C)cc6c6cc(C(C)(C)C)ccc65)cc(c42)N3c2ccc(-c3ccccc3)cc2)c1 ; CC(C)(C)c1ccc(N(c2ccc(C(C)(C)C)cc2)c2cc3c4c(c2)N(c2ccc(-c5ccccc5)cc2)c2ncn(-c5ccccc5)c2B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3ncn(-c6ccccc6)c3B5c3cc(-c5ccccc5)ccc3N4c3ccc(-c4ccccc4)cc3)cc2)cc1]

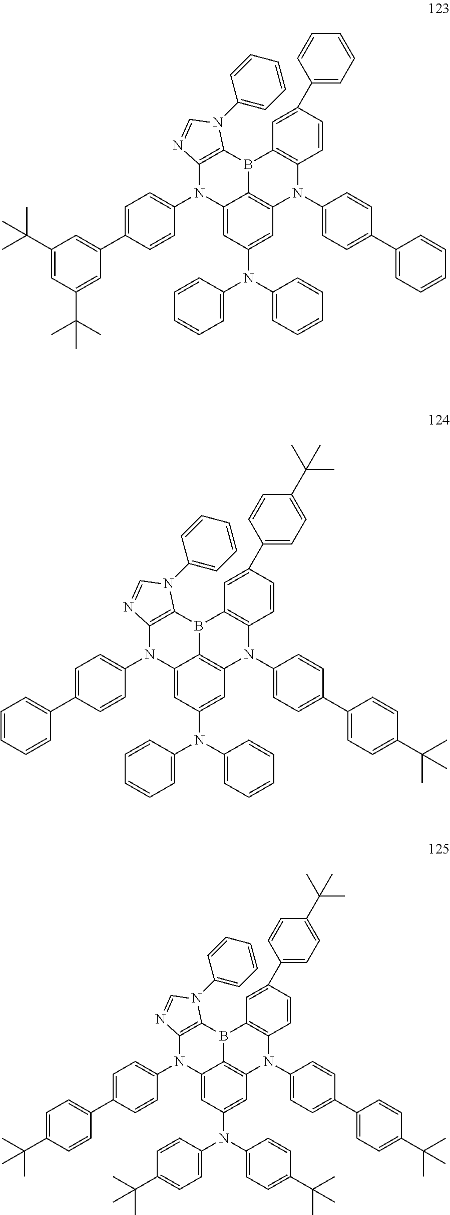

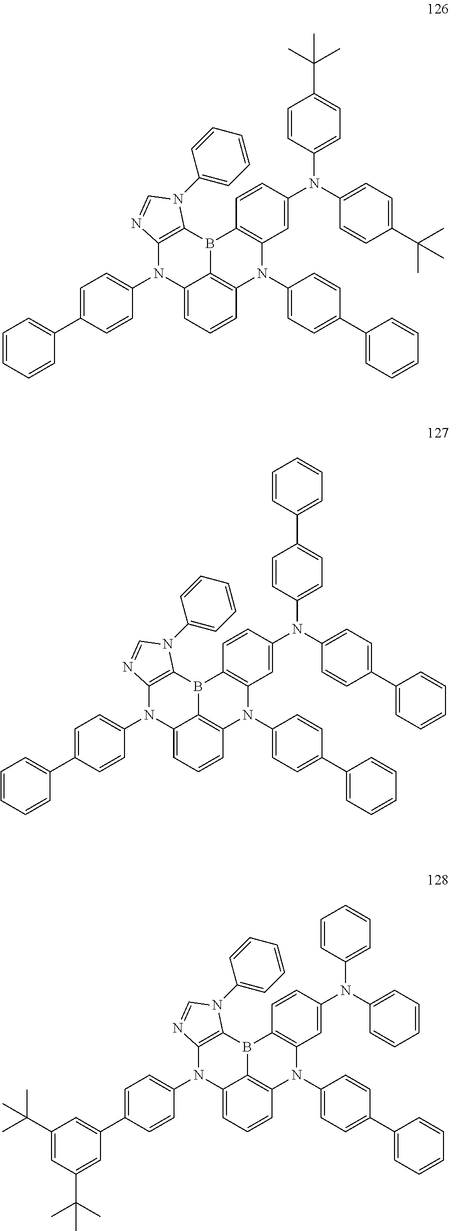

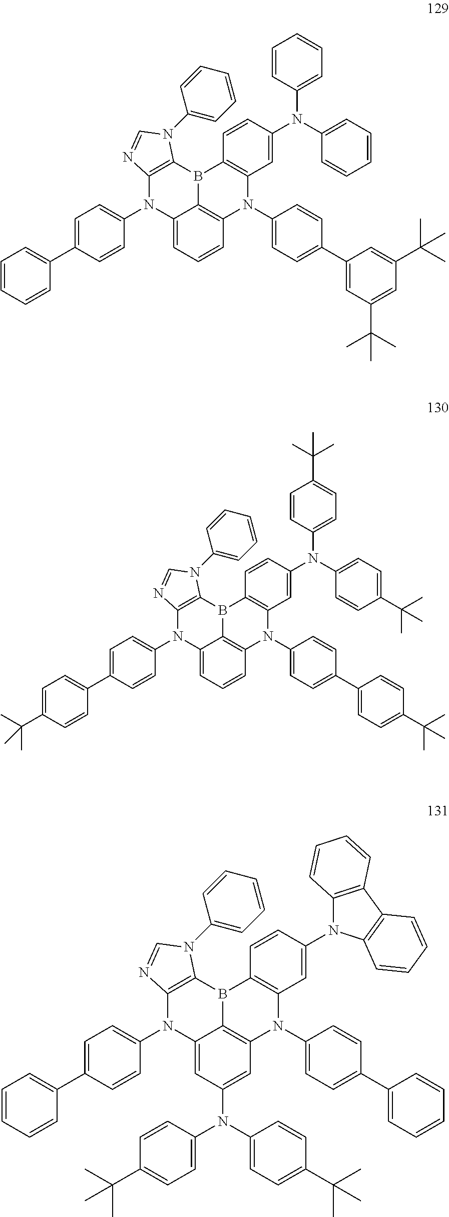

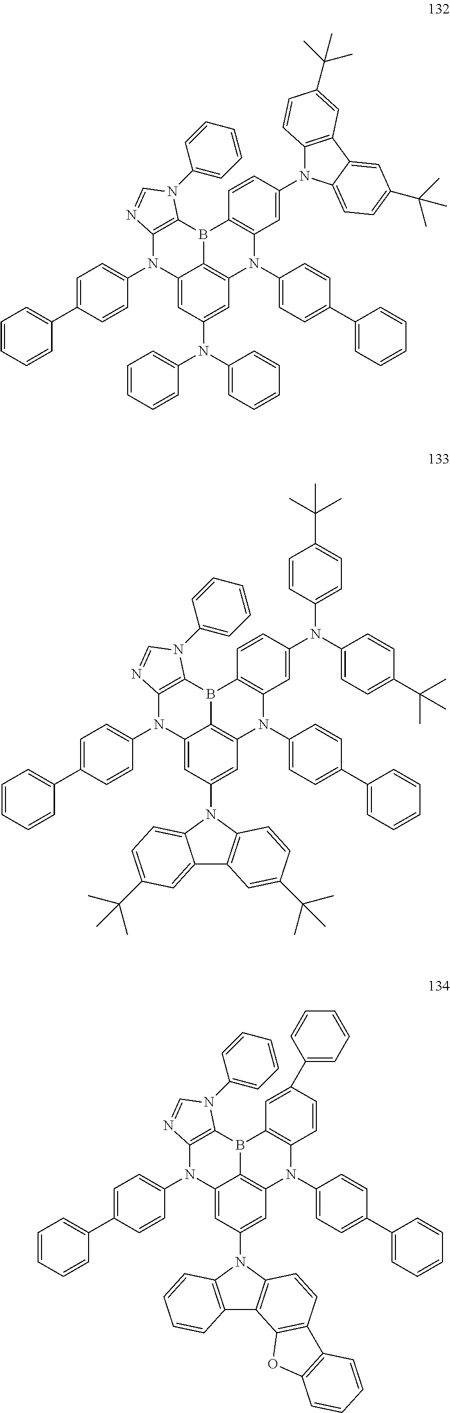 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(ncn1-c1ccccc1)N3c1ccc(-c2ccccc2)cc1 ; CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2c(ncn2-c2ccccc2)N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3ncn(-c5ccccc5)c34)cc2)cc1]

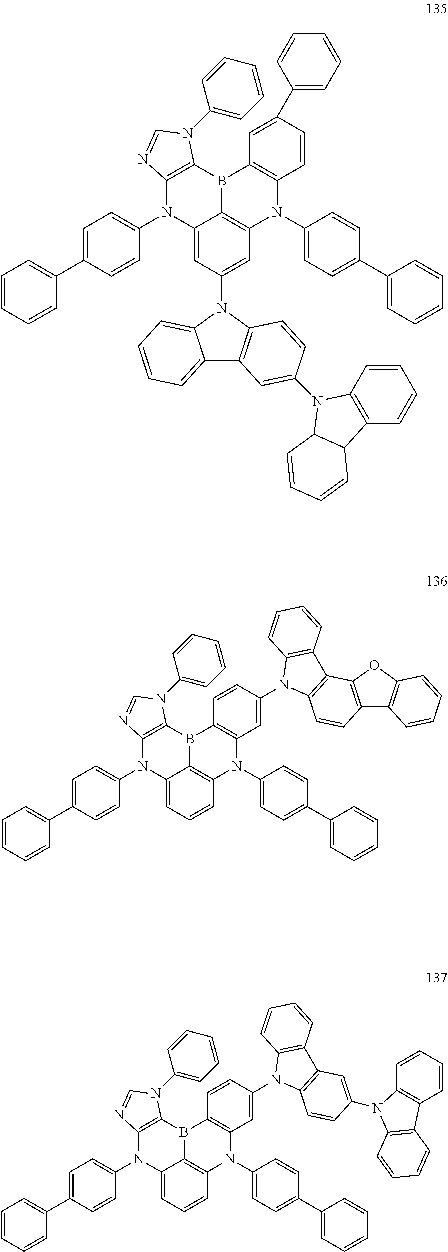 [SMILES: C1=CC2c3ccccc3N(c3ccc4c(c3)c3ccccc3n4-c3cc4c5c(c3)N(c3ccc(-c6ccccc6)cc3)c3ncn(-c6ccccc6)c3B5c3cc(-c5ccccc5)ccc3N4c3ccc(-c4ccccc4)cc3)C2C=C1 ; c1ccc(-c2ccc(N3c4cc(-n5c6ccccc6c6c7oc8ccccc8c7ccc65)ccc4B4c5c3cccc5N(c3ccc(-c5ccccc5)cc3)c3ncn(-c5ccccc5)c34)cc2)cc1 ; c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3ncn(-c5ccccc5)c34)cc2)cc1]

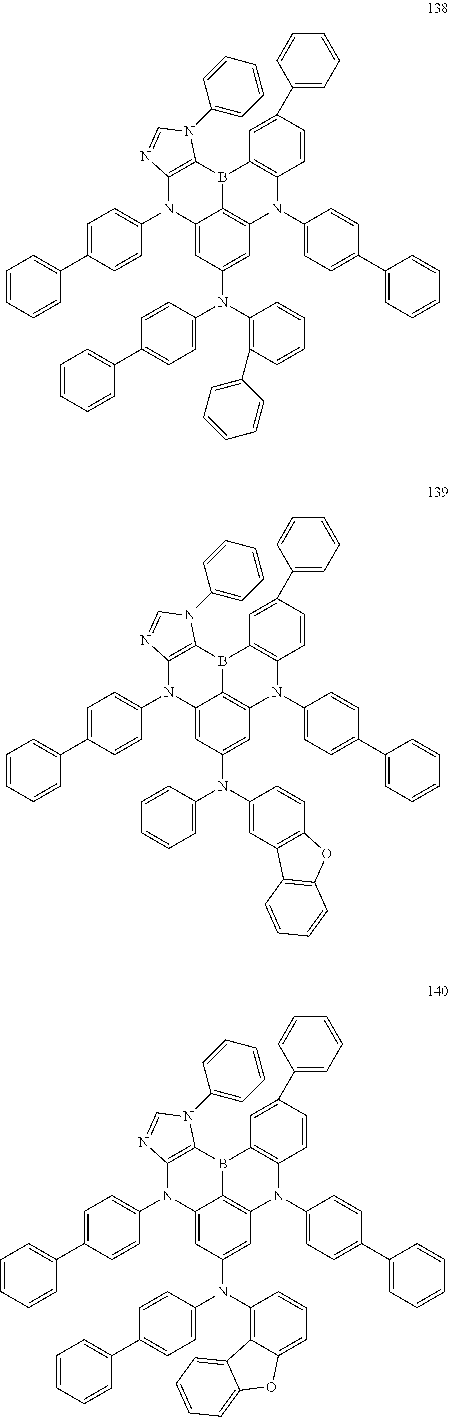 [SMILES: c1ccc(-c2ccc(N(c3cc4c5c(c3)N(c3ccc(-c6ccccc6)cc3)c3ncn(-c6ccccc6)c3B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3ncn(-c5ccccc5)c34)cc2)cc1 ; c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3ncn(-c5ccccc5)c34)cc2)cc1]

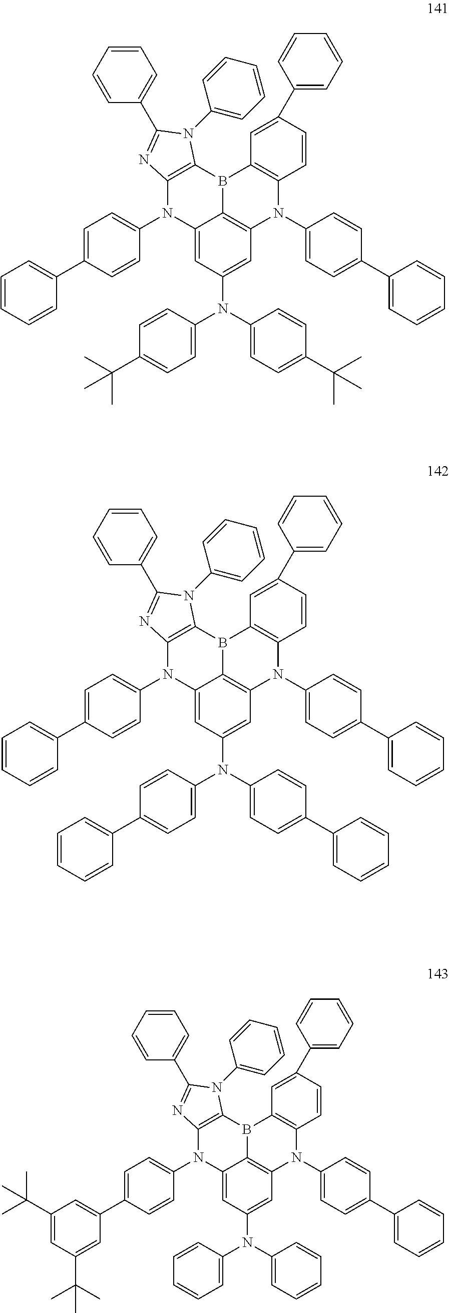 [SMILES: CC(C)(C)c1ccc(N(c2ccc(C(C)(C)C)cc2)c2cc3c4c(c2)N(c2ccc(-c5ccccc5)cc2)c2nc(-c5ccccc5)n(-c5ccccc5)c2B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3nc(-c6ccccc6)n(-c6ccccc6)c3B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c3nc(-c3ccccc3)n4-c3ccccc3)cc2)cc(C(C)(C)C)c1]

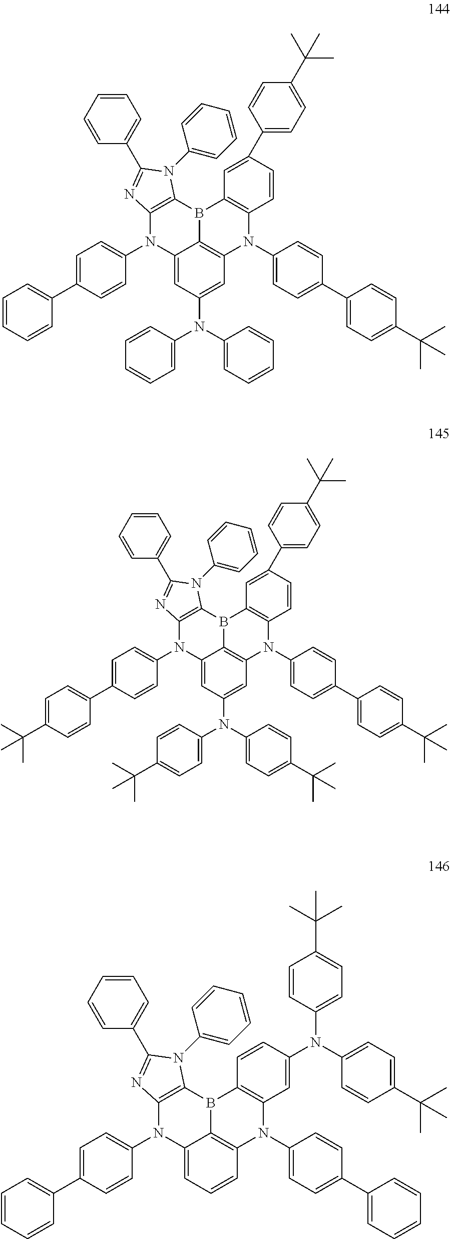

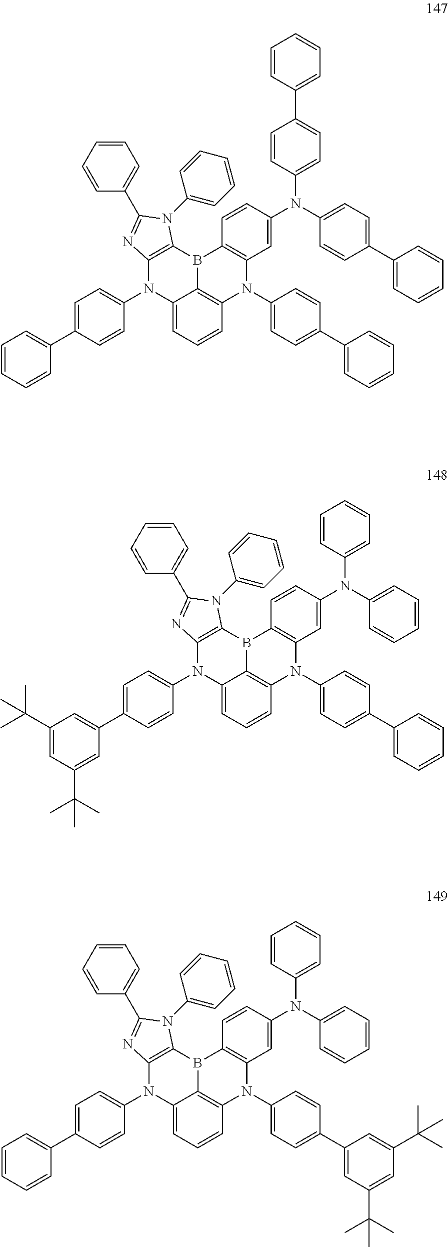 [SMILES: c1ccc(-c2ccc(N(c3ccc(-c4ccccc4)cc3)c3ccc4c(c3)N(c3ccc(-c5ccccc5)cc3)c3cccc5c3B4c3c(nc(-c4ccccc4)n3-c3ccccc3)N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4c3nc(-c3ccccc3)n4-c3ccccc3)cc2)cc(C(C)(C)C)c1 ; CC(C)(C)c1cc(-c2ccc(N3c4cc(N(c5ccccc5)c5ccccc5)ccc4B4c5c3cccc5N(c3ccc(-c5ccccc5)cc3)c3nc(-c5ccccc5)n(-c5ccccc5)c34)cc2)cc(C(C)(C)C)c1]

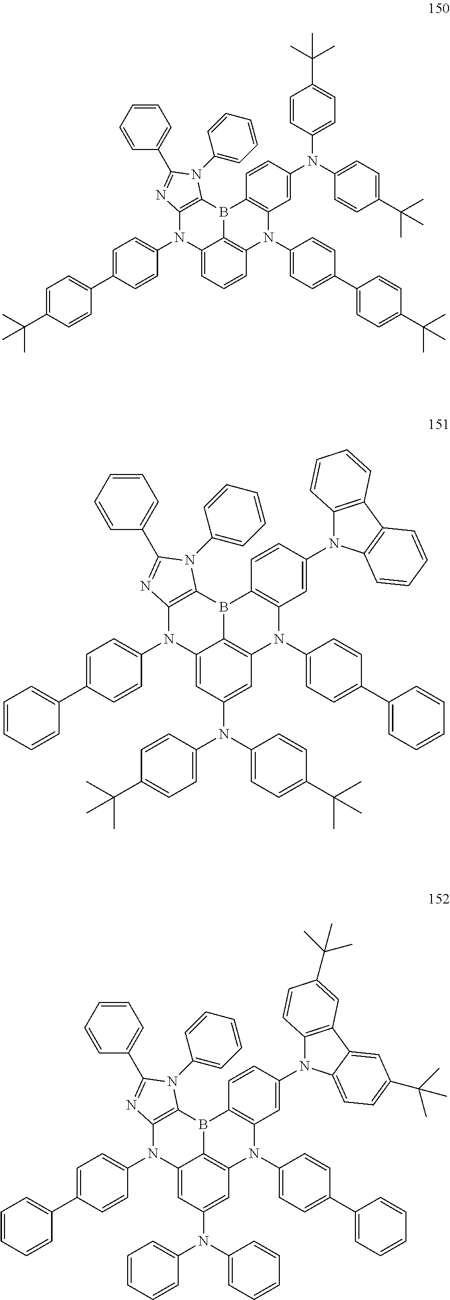 [SMILES: CC(C)(C)c1ccc(-c2ccc(N3c4cc(N(c5ccc(C(C)(C)C)cc5)c5ccc(C(C)(C)C)cc5)ccc4B4c5c3cccc5N(c3ccc(-c5ccc(C(C)(C)C)cc5)cc3)c3nc(-c5ccccc5)n(-c5ccccc5)c34)cc2)cc1 ; CC(C)(C)c1ccc(N(c2ccc(C(C)(C)C)cc2)c2cc3c4c(c2)N(c2ccc(-c5ccccc5)cc2)c2nc(-c5ccccc5)n(-c5ccccc5)c2B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(nc(-c2ccccc2)n1-c1ccccc1)N3c1ccc(-c2ccccc2)cc1]

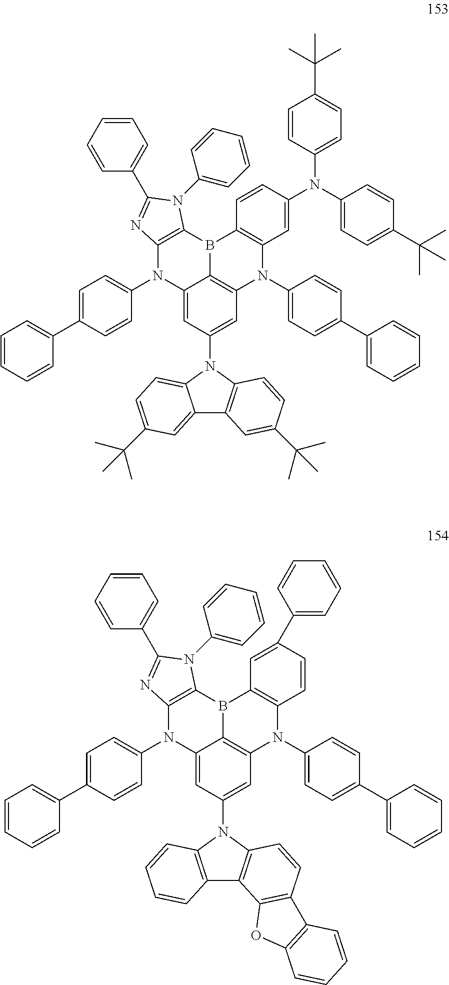 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2c(nc(-c3ccccc3)n2-c2ccccc2)N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3nc(-c5ccccc5)n(-c5ccccc5)c34)cc2)cc1]

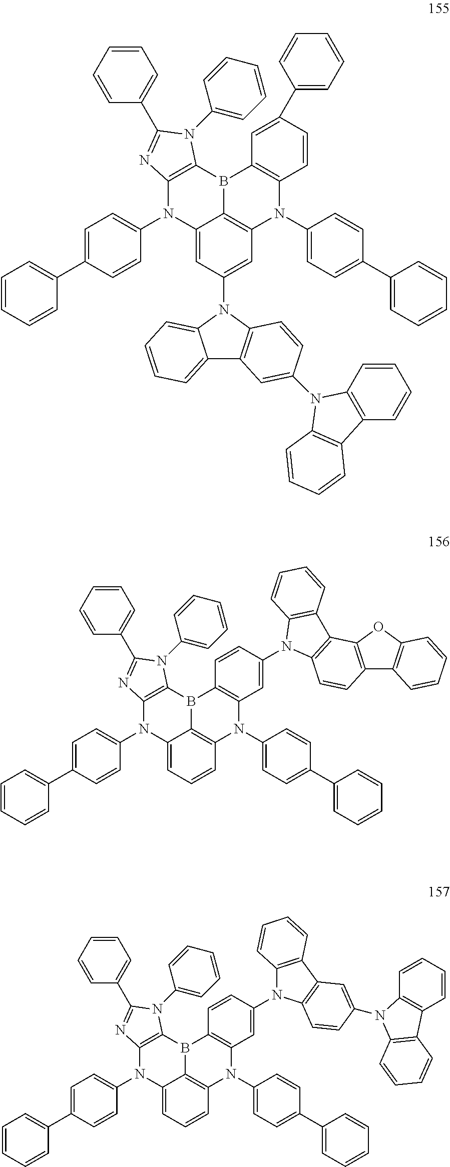 [SMILES: c1ccc(-c2ccc(N3c4ccc(-c5ccccc5)cc4B4c5c3cc(-n3c6ccccc6c6cc(-n7c8ccccc8c8ccccc87)ccc63)cc5N(c3ccc(-c5ccccc5)cc3)c3nc(-c5ccccc5)n(-c5ccccc5)c34)cc2)cc1 ; c1ccc(-c2ccc(N3c4cc(-n5c6ccccc6c6c7oc8ccccc8c7ccc65)ccc4B4c5c3cccc5N(c3ccc(-c5ccccc5)cc3)c3nc(-c5ccccc5)n(-c5ccccc5)c34)cc2)cc1 ; c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3nc(-c5ccccc5)n(-c5ccccc5)c34)cc2)cc1]

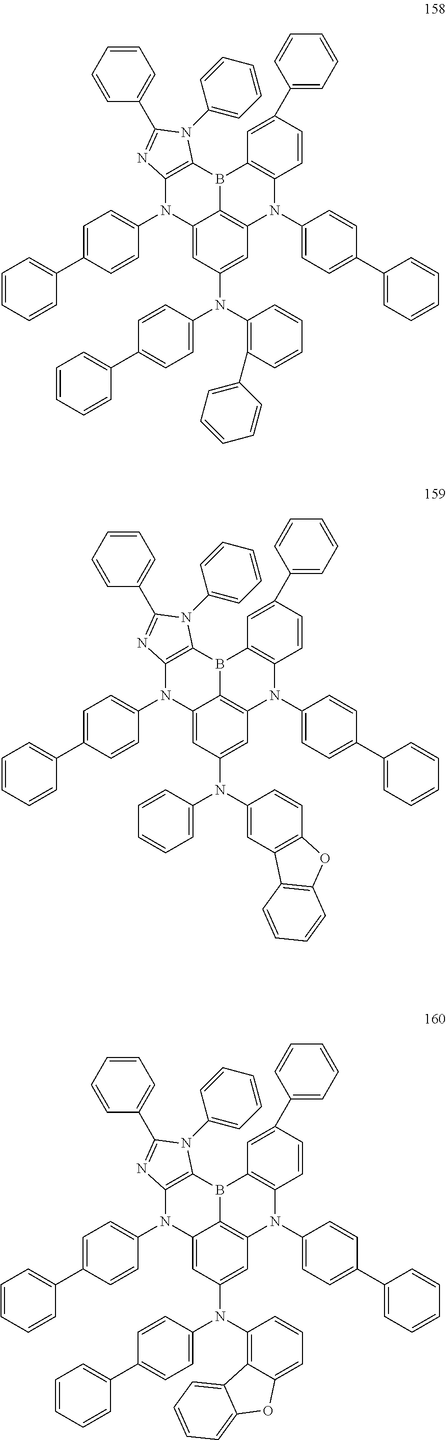 [SMILES: c1ccc(-c2ccc(N(c3cc4c5c(c3)N(c3ccc(-c6ccccc6)cc3)c3nc(-c6ccccc6)n(-c6ccccc6)c3B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3nc(-c5ccccc5)n(-c5ccccc5)c34)cc2)cc1 ; c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3nc(-c5ccccc5)n(-c5ccccc5)c34)cc2)cc1]

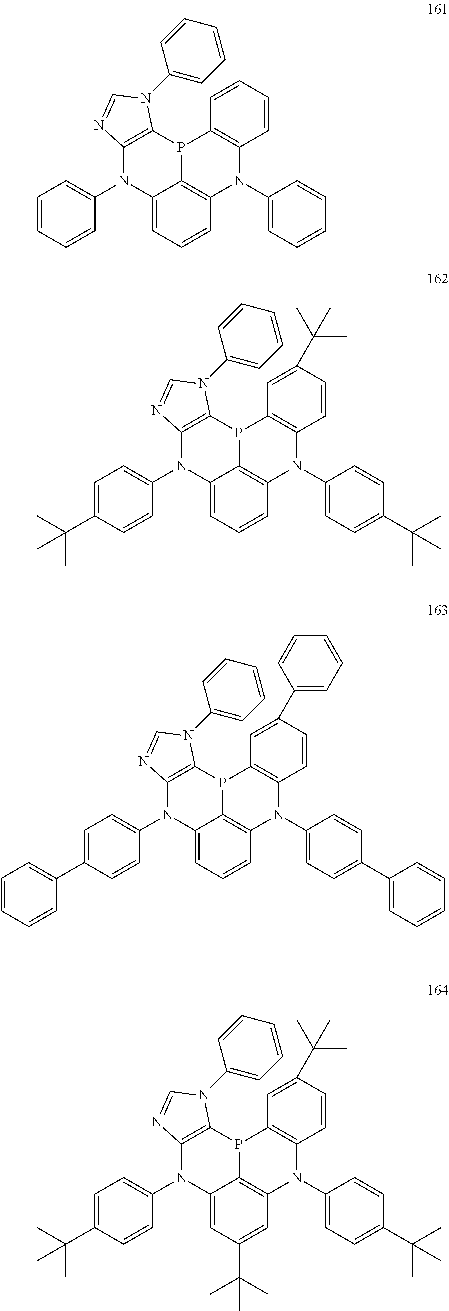

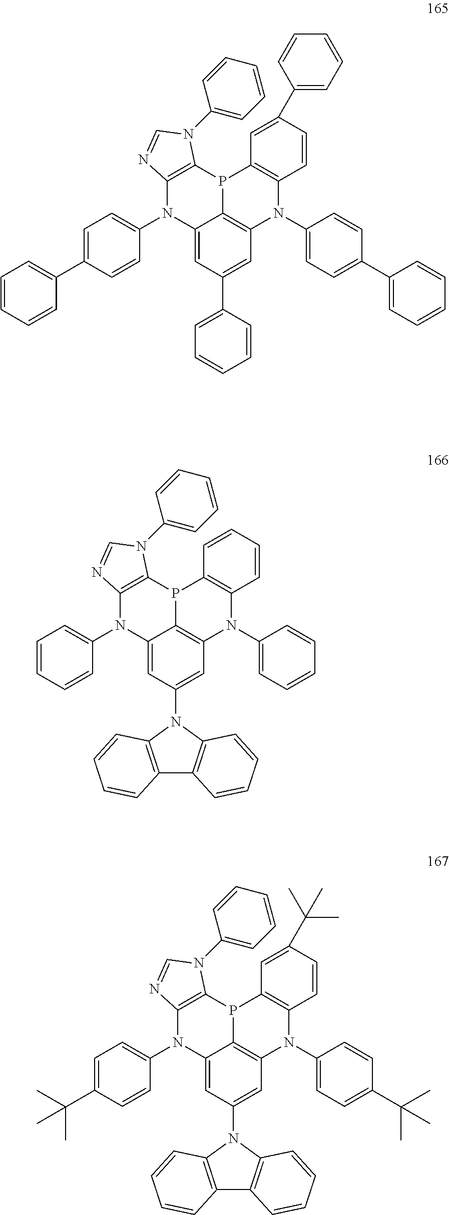 [SMILES: c1ccc(-c2ccc(N3c4ccc(-c5ccccc5)cc4P4c5c3cc(-c3ccccc3)cc5N(c3ccc(-c5ccccc5)cc3)c3ncn(-c5ccccc5)c34)cc2)cc1 ; c1ccc(N2c3ccccc3P3c4c2cc(-n2c5ccccc5c5ccccc52)cc4N(c2ccccc2)c2ncn(-c4ccccc4)c23)cc1 ; CC(C)(C)c1ccc(N2c3ccc(C(C)(C)C)cc3P3c4c2cc(-n2c5ccccc5c5ccccc52)cc4N(c2ccc(C(C)(C)C)cc2)c2ncn(-c4ccccc4)c23)cc1]

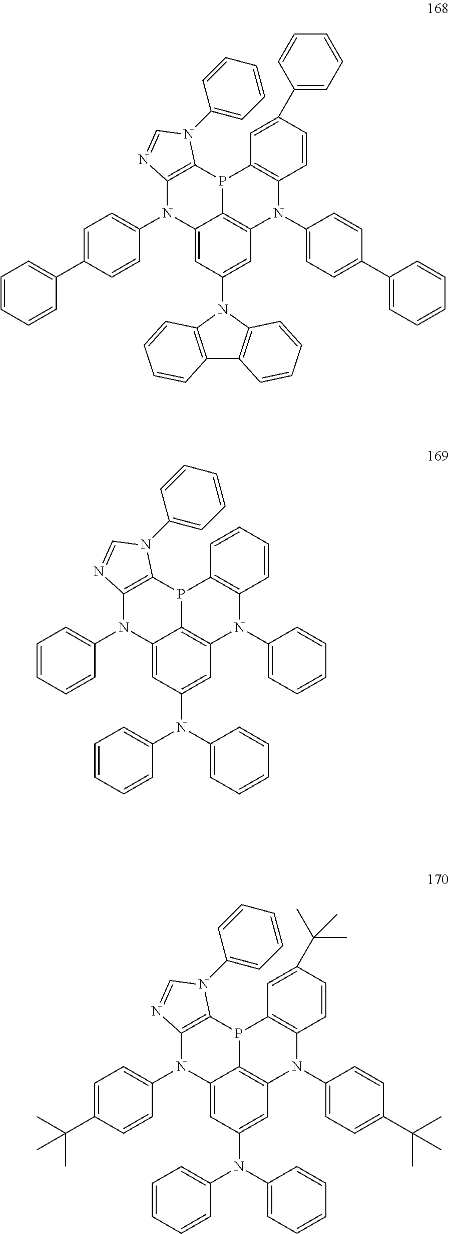 [SMILES: c1ccc(-c2ccc(N3c4ccc(-c5ccccc5)cc4P4c5c3cc(-n3c6ccccc6c6ccccc63)cc5N(c3ccc(-c5ccccc5)cc3)c3ncn(-c5ccccc5)c34)cc2)cc1 ; c1ccc(N(c2ccccc2)c2cc3c4c(c2)N(c2ccccc2)c2ncn(-c5ccccc5)c2P4c2ccccc2N3c2ccccc2)cc1 ; CC(C)(C)c1ccc(N2c3ccc(C(C)(C)C)cc3P3c4c2cc(N(c2ccccc2)c2ccccc2)cc4N(c2ccc(C(C)(C)C)cc2)c2ncn(-c4ccccc4)c23)cc1]

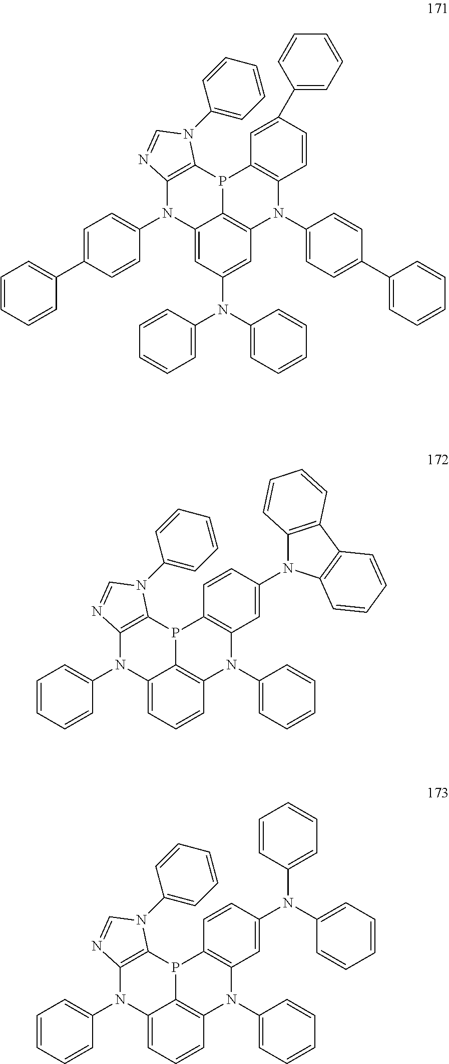 [SMILES: c1ccc(-c2ccc(N3c4ccc(-c5ccccc5)cc4P4c5c3cc(N(c3ccccc3)c3ccccc3)cc5N(c3ccc(-c5ccccc5)cc3)c3ncn(-c5ccccc5)c34)cc2)cc1 ; c1ccc(N2c3cc(-n4c5ccccc5c5ccccc54)ccc3P3c4c2cccc4N(c2ccccc2)c2ncn(-c4ccccc4)c23)cc1 ; c1ccc(N(c2ccccc2)c2ccc3c(c2)N(c2ccccc2)c2cccc4c2P3c2c(ncn2-c2ccccc2)N4c2ccccc2)cc1]

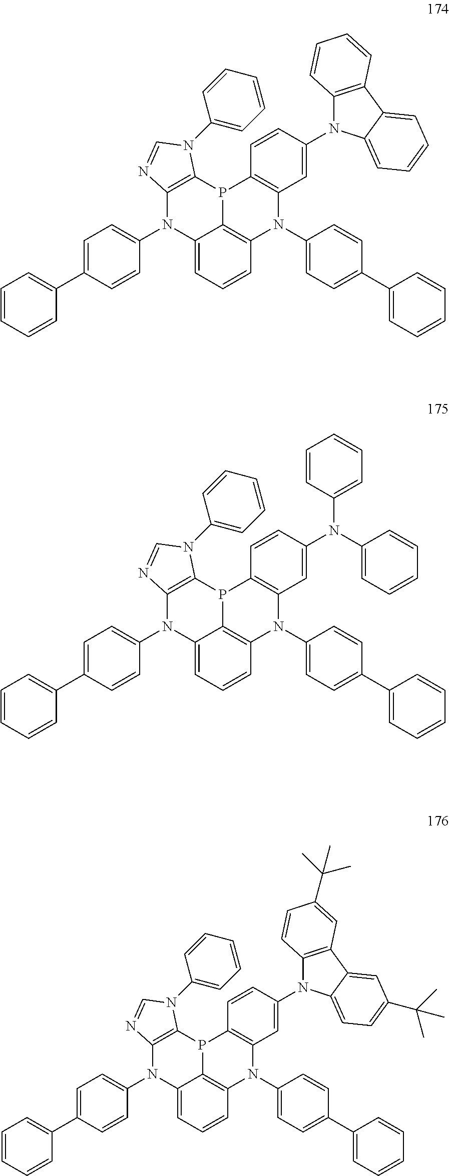 [SMILES: c1ccc(-c2ccc(N3c4cc(-n5c6ccccc6c6ccccc65)ccc4P4c5c3cccc5N(c3ccc(-c5ccccc5)cc3)c3ncn(-c5ccccc5)c34)cc2)cc1 ; c1ccc(-c2ccc(N3c4cc(N(c5ccccc5)c5ccccc5)ccc4P4c5c3cccc5N(c3ccc(-c5ccccc5)cc3)c3ncn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1c(ncn1-c1ccccc1)N3c1ccc(-c2ccccc2)cc1]

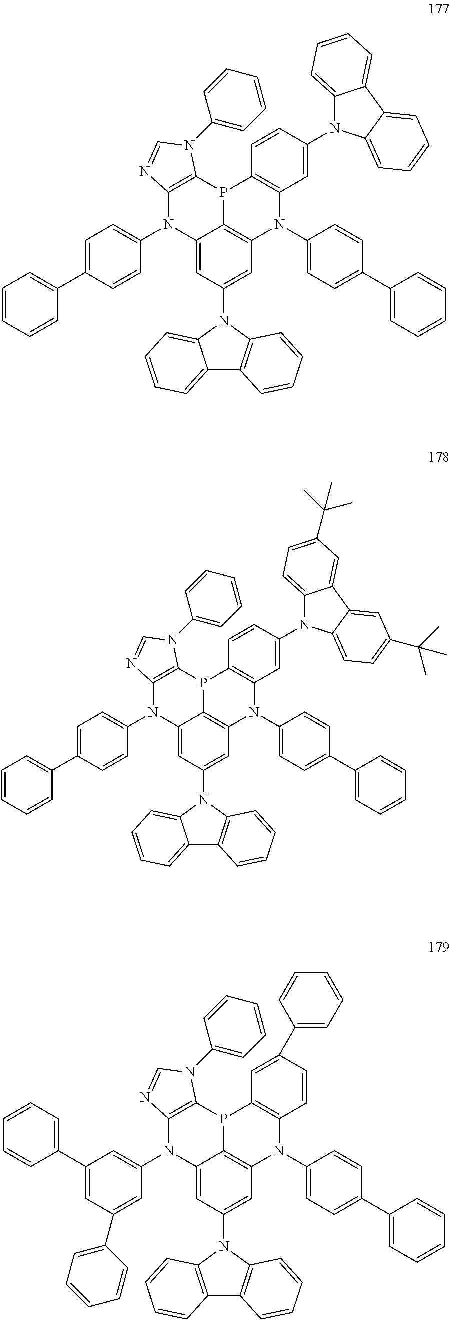 [SMILES: c1ccc(-c2ccc(N3c4cc(-n5c6ccccc6c6ccccc65)ccc4P4c5c3cc(-n3c6ccccc6c6ccccc63)cc5N(c3ccc(-c5ccccc5)cc3)c3ncn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1c(ncn1-c1ccccc1)N3c1ccc(-c2ccccc2)cc1 ; c1ccc(-c2ccc(N3c4ccc(-c5ccccc5)cc4P4c5c3cc(-n3c6ccccc6c6ccccc63)cc5N(c3cc(-c5ccccc5)cc(-c5ccccc5)c3)c3ncn(-c5ccccc5)c34)cc2)cc1]

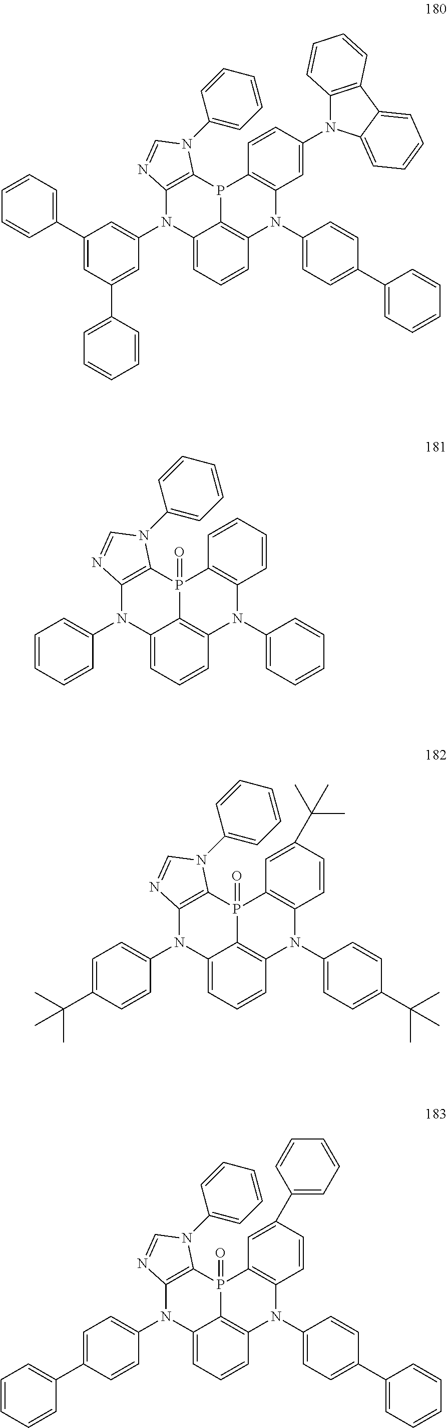 [SMILES: c1ccc(-c2ccc(N3c4cc(-n5c6ccccc6c6ccccc65)ccc4P4c5c3cccc5N(c3cc(-c5ccccc5)cc(-c5ccccc5)c3)c3ncn(-c5ccccc5)c34)cc2)cc1 ; O=P12c3ccccc3N(c3ccccc3)c3cccc(c31)N(c1ccccc1)c1ncn(-c3ccccc3)c12 ; CC(C)(C)c1ccc(N2c3ccc(C(C)(C)C)cc3P3(=O)c4c2cccc4N(c2ccc(C(C)(C)C)cc2)c2ncn(-c4ccccc4)c23)cc1 ; O=P12c3cc(-c4ccccc4)ccc3N(c3ccc(-c4ccccc4)cc3)c3cccc(c31)N(c1ccc(-c3ccccc3)cc1)c1ncn(-c3ccccc3)c12]

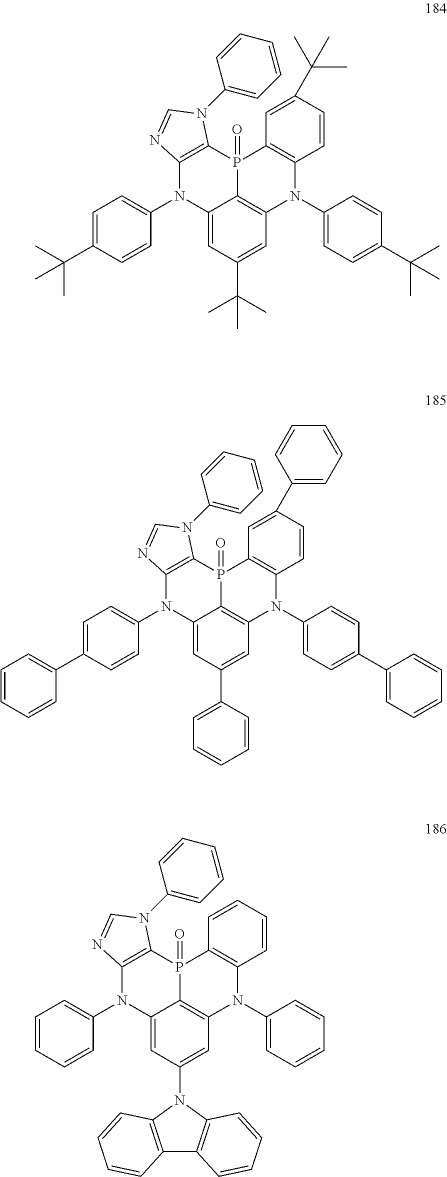 [SMILES: CC(C)(C)c1ccc(N2c3ccc(C(C)(C)C)cc3P3(=O)c4c2cc(C(C)(C)C)cc4N(c2ccc(C(C)(C)C)cc2)c2ncn(-c4ccccc4)c23)cc1 ; O=P12c3cc(-c4ccccc4)ccc3N(c3ccc(-c4ccccc4)cc3)c3cc(-c4ccccc4)cc(c31)N(c1ccc(-c3ccccc3)cc1)c1ncn(-c3ccccc3)c12 ; O=P12c3ccccc3N(c3ccccc3)c3cc(-n4c5ccccc5c5ccccc54)cc(c31)N(c1ccccc1)c1ncn(-c3ccccc3)c12]

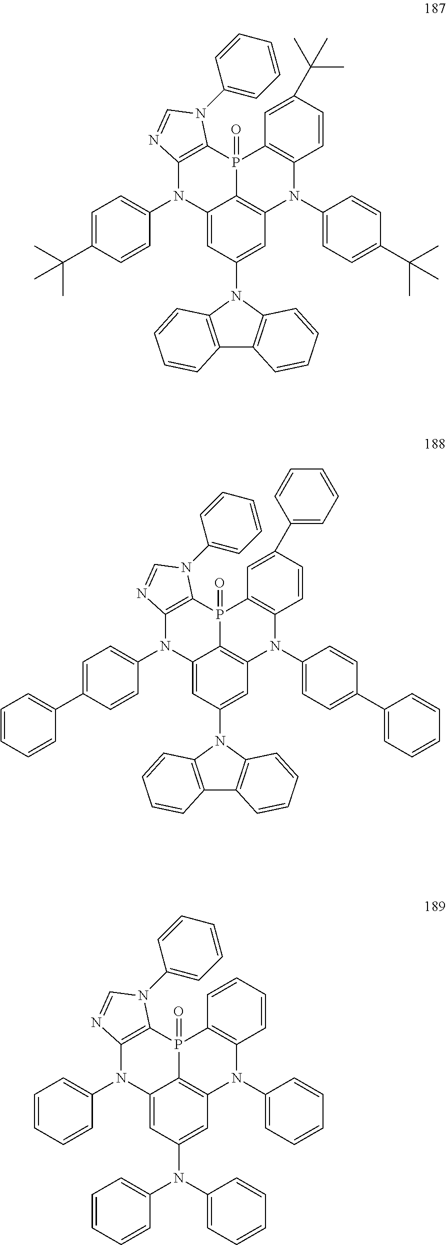 [SMILES: CC(C)(C)c1ccc(N2c3ccc(C(C)(C)C)cc3P3(=O)c4c2cc(-n2c5ccccc5c5ccccc52)cc4N(c2ccc(C(C)(C)C)cc2)c2ncn(-c4ccccc4)c23)cc1 ; O=P12c3cc(-c4ccccc4)ccc3N(c3ccc(-c4ccccc4)cc3)c3cc(-n4c5ccccc5c5ccccc54)cc(c31)N(c1ccc(-c3ccccc3)cc1)c1ncn(-c3ccccc3)c12 ; O=P12c3ccccc3N(c3ccccc3)c3cc(N(c4ccccc4)c4ccccc4)cc(c31)N(c1ccccc1)c1ncn(-c3ccccc3)c12]

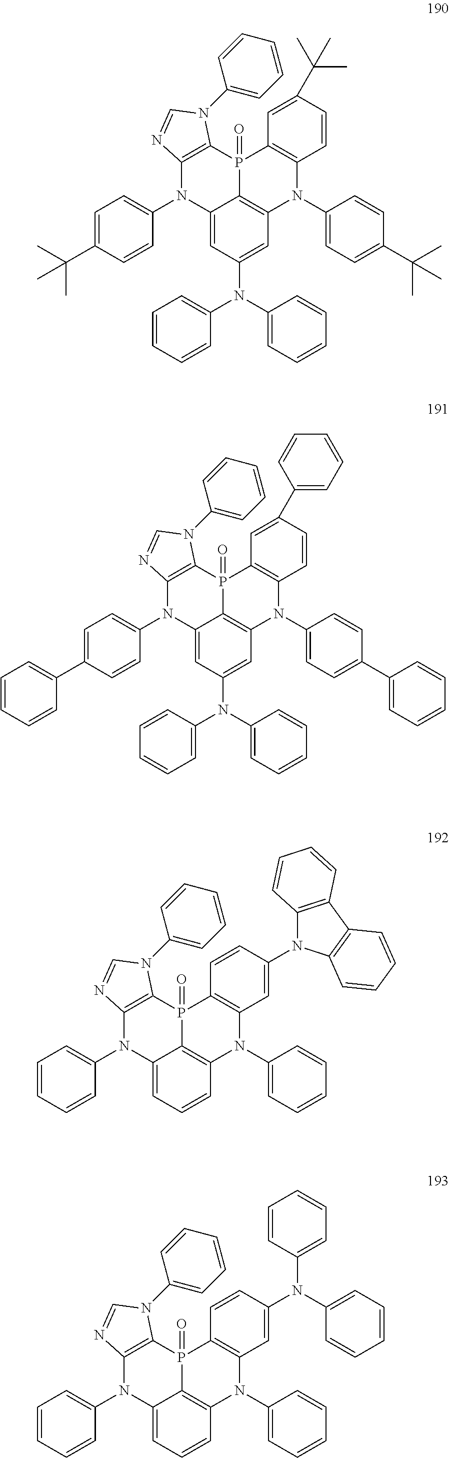 [SMILES: CC(C)(C)c1ccc(N2c3ccc(C(C)(C)C)cc3P3(=O)c4c2cc(N(c2ccccc2)c2ccccc2)cc4N(c2ccc(C(C)(C)C)cc2)c2ncn(-c4ccccc4)c23)cc1 ; O=P12c3cc(-c4ccccc4)ccc3N(c3ccc(-c4ccccc4)cc3)c3cc(N(c4ccccc4)c4ccccc4)cc(c31)N(c1ccc(-c3ccccc3)cc1)c1ncn(-c3ccccc3)c12 ; O=P12c3ccc(-n4c5ccccc5c5ccccc54)cc3N(c3ccccc3)c3cccc(c31)N(c1ccccc1)c1ncn(-c3ccccc3)c12 ; O=P12c3ccc(N(c4ccccc4)c4ccccc4)cc3N(c3ccccc3)c3cccc(c31)N(c1ccccc1)c1ncn(-c3ccccc3)c12]

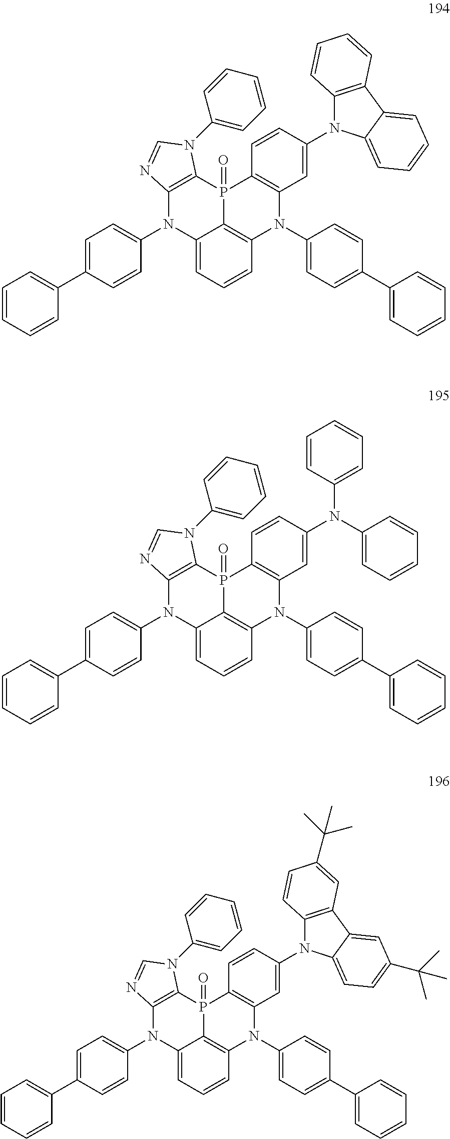 [SMILES: O=P12c3ccc(-n4c5ccccc5c5ccccc54)cc3N(c3ccc(-c4ccccc4)cc3)c3cccc(c31)N(c1ccc(-c3ccccc3)cc1)c1ncn(-c3ccccc3)c12 ; O=P12c3ccc(N(c4ccccc4)c4ccccc4)cc3N(c3ccc(-c4ccccc4)cc3)c3cccc(c31)N(c1ccc(-c3ccccc3)cc1)c1ncn(-c3ccccc3)c12 ; CC(C)(C)c1ccc2c(c1)c1cc(C(C)(C)C)ccc1n2-c1ccc2c(c1)N(c1ccc(-c3ccccc3)cc1)c1cccc3c1P2(=O)c1c(ncn1-c1ccccc1)N3c1ccc(-c2ccccc2)cc1]

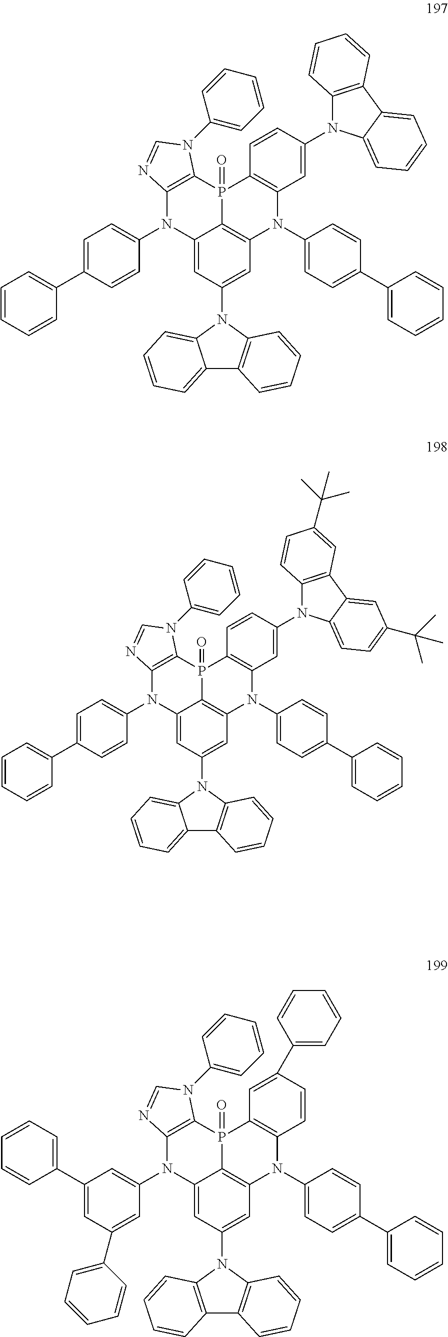 [SMILES: O=P12c3ccc(-n4c5ccccc5c5ccccc54)cc3N(c3ccc(-c4ccccc4)cc3)c3cc(-n4c5ccccc5c5ccccc54)cc(c31)N(c1ccc(-c3ccccc3)cc1)c1ncn(-c3ccccc3)c12 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2(=O)c1c(ncn1-c1ccccc1)N3c1ccc(-c2ccccc2)cc1 ; O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1ncn(-c3ccccc3)c12]

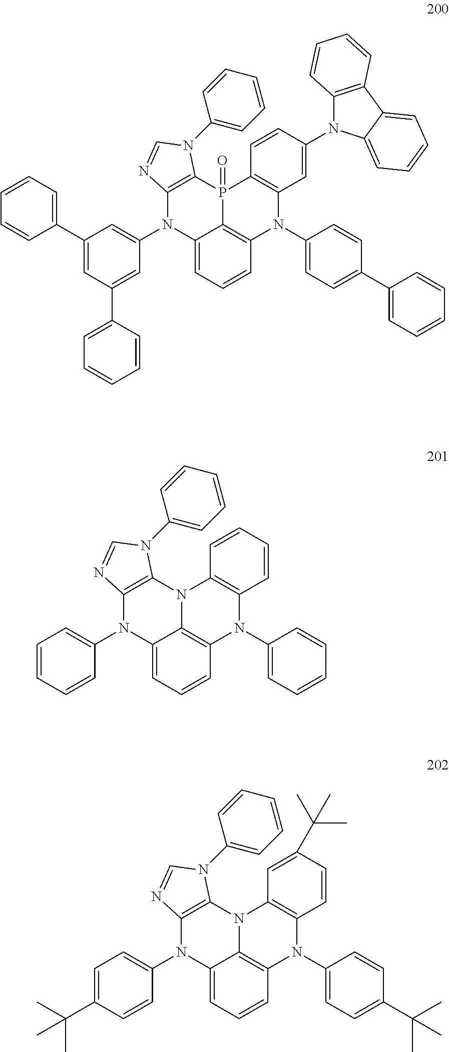 [SMILES: O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1ncn(-c3ccccc3)c12 ; c1ccc(N2c3ccccc3N3c4c2cccc4N(c2ccccc2)c2ncn(-c4ccccc4)c23)cc1 ; CC(C)(C)c1ccc(N2c3ccc(C(C)(C)C)cc3N3c4c2cccc4N(c2ccc(C(C)(C)C)cc2)c2ncn(-c4ccccc4)c23)cc1]

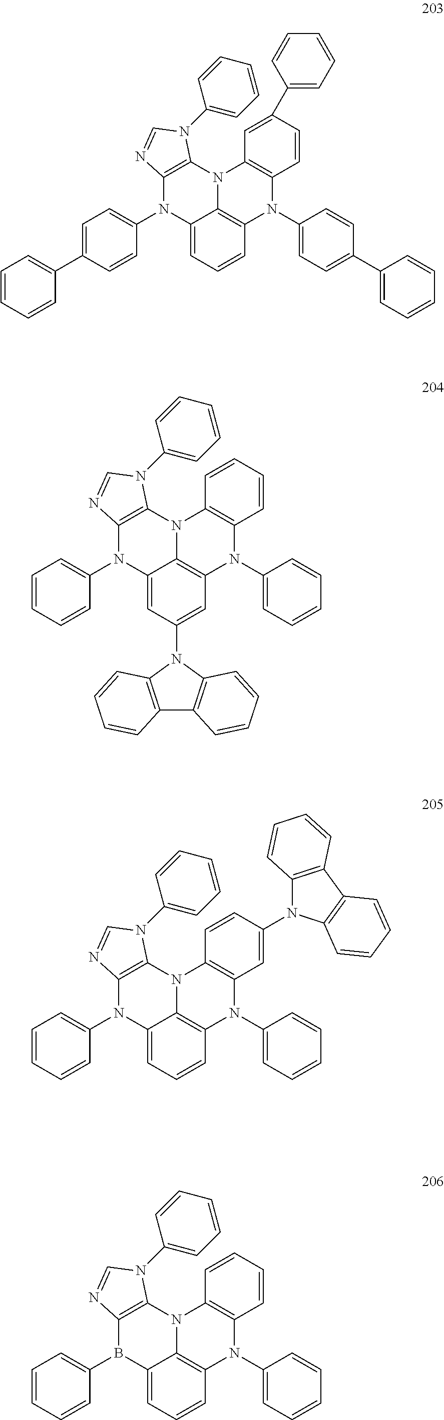 [SMILES: c1ccc(-c2ccc(N3c4ccc(-c5ccccc5)cc4N4c5c3cccc5N(c3ccc(-c5ccccc5)cc3)c3ncn(-c5ccccc5)c34)cc2)cc1 ; c1ccc(N2c3ccccc3N3c4c2cc(-n2c5ccccc5c5ccccc52)cc4N(c2ccccc2)c2ncn(-c4ccccc4)c23)cc1 ; c1ccc(N2c3cc(-n4c5ccccc5c5ccccc54)ccc3N3c4c2cccc4N(c2ccccc2)c2ncn(-c4ccccc4)c23)cc1 ; c1ccc(B2c3cccc4c3N(c3ccccc3N4c3ccccc3)c3c2ncn3-c2ccccc2)cc1]

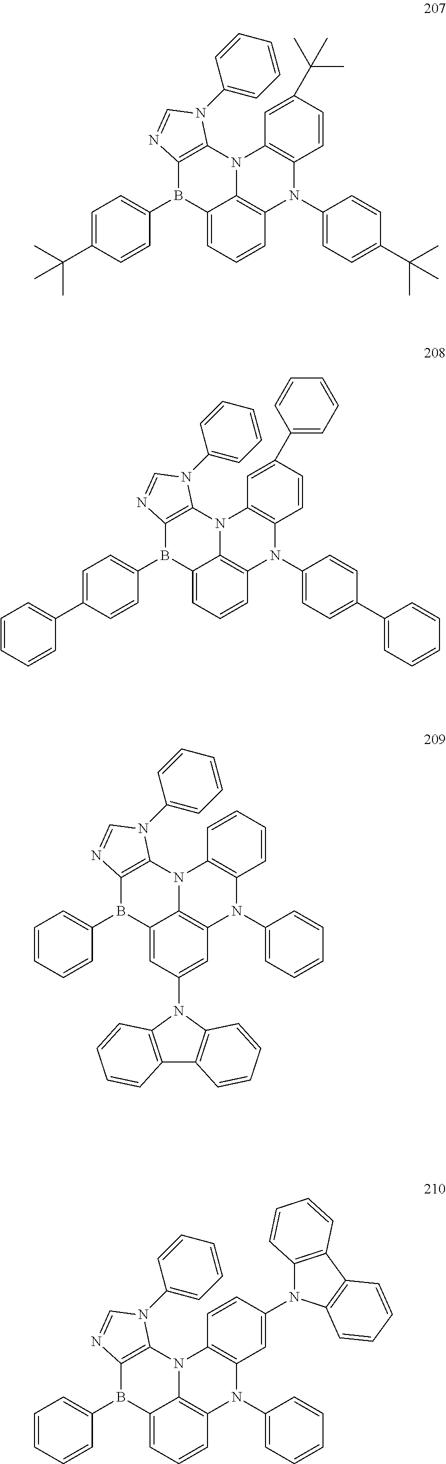 [SMILES: CC(C)(C)c1ccc(B2c3cccc4c3N(c3cc(C(C)(C)C)ccc3N4c3ccc(C(C)(C)C)cc3)c3c2ncn3-c2ccccc2)cc1 ; c1ccc(-c2ccc(B3c4cccc5c4N(c4cc(-c6ccccc6)ccc4N5c4ccc(-c5ccccc5)cc4)c4c3ncn4-c3ccccc3)cc2)cc1 ; c1ccc(B2c3cc(-n4c5ccccc5c5ccccc54)cc4c3N(c3ccccc3N4c3ccccc3)c3c2ncn3-c2ccccc2)cc1 ; c1ccc(B2c3cccc4c3N(c3ccc(-n5c6ccccc6c6ccccc65)cc3N4c3ccccc3)c3c2ncn3-c2ccccc2)cc1]

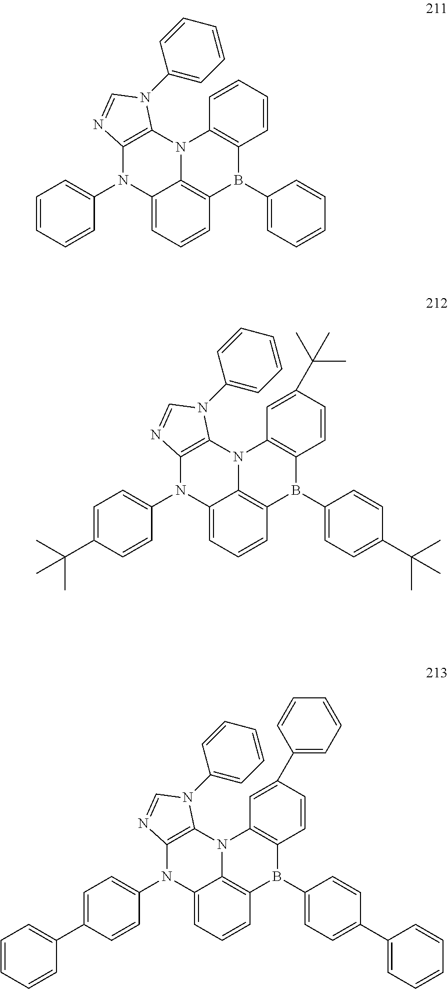 [SMILES: c1ccc(B2c3ccccc3N3c4c2cccc4N(c2ccccc2)c2ncn(-c4ccccc4)c23)cc1 ; CC(C)(C)c1ccc(B2c3ccc(C(C)(C)C)cc3N3c4c2cccc4N(c2ccc(C(C)(C)C)cc2)c2ncn(-c4ccccc4)c23)cc1 ; c1ccc(-c2ccc(B3c4ccc(-c5ccccc5)cc4N4c5c3cccc5N(c3ccc(-c5ccccc5)cc3)c3ncn(-c5ccccc5)c34)cc2)cc1]

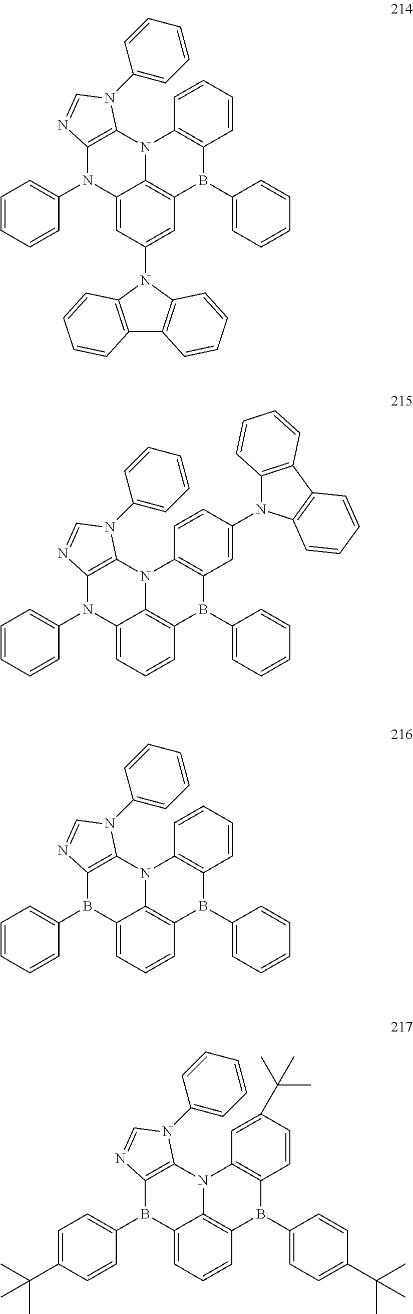 [SMILES: c1ccc(B2c3ccccc3N3c4c2cc(-n2c5ccccc5c5ccccc52)cc4N(c2ccccc2)c2ncn(-c4ccccc4)c23)cc1 ; c1ccc(B2c3cc(-n4c5ccccc5c5ccccc54)ccc3N3c4c2cccc4N(c2ccccc2)c2ncn(-c4ccccc4)c23)cc1 ; c1ccc(B2c3ccccc3N3c4c2cccc4B(c2ccccc2)c2ncn(-c4ccccc4)c23)cc1 ; CC(C)(C)c1ccc(B2c3ccc(C(C)(C)C)cc3N3c4c2cccc4B(c2ccc(C(C)(C)C)cc2)c2ncn(-c4ccccc4)c23)cc1]

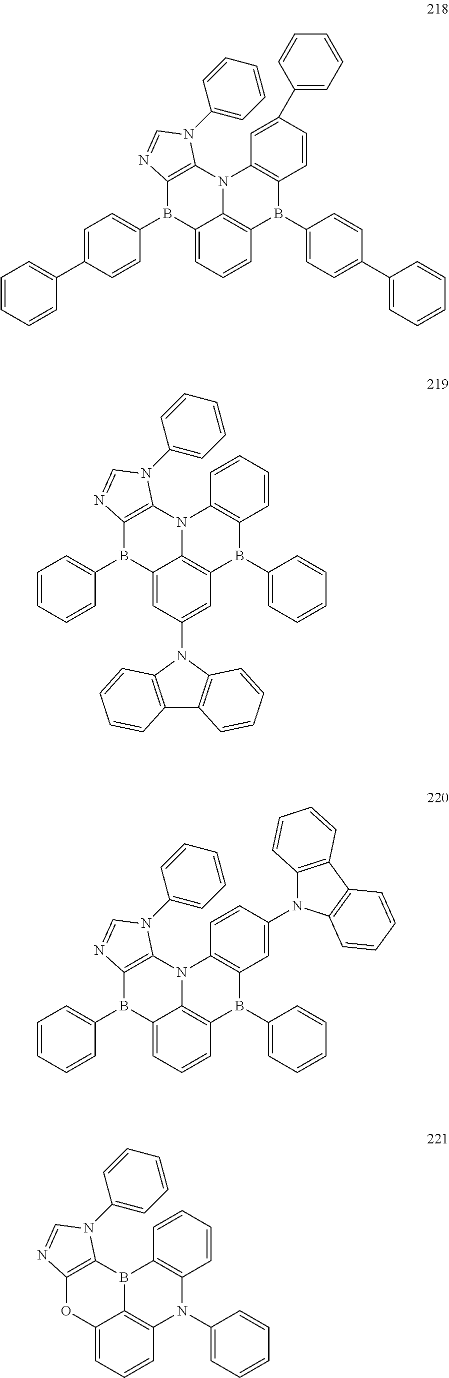 [SMILES: c1ccc(-c2ccc(B3c4ccc(-c5ccccc5)cc4N4c5c3cccc5B(c3ccc(-c5ccccc5)cc3)c3ncn(-c5ccccc5)c34)cc2)cc1 ; c1ccc(B2c3ccccc3N3c4c2cc(-n2c5ccccc5c5ccccc52)cc4B(c2ccccc2)c2ncn(-c4ccccc4)c23)cc1 ; c1ccc(B2c3cc(-n4c5ccccc5c5ccccc54)ccc3N3c4c2cccc4B(c2ccccc2)c2ncn(-c4ccccc4)c23)cc1 ; c1ccc(N2c3ccccc3B3c4c(cccc42)Oc2ncn(-c4ccccc4)c23)cc1]

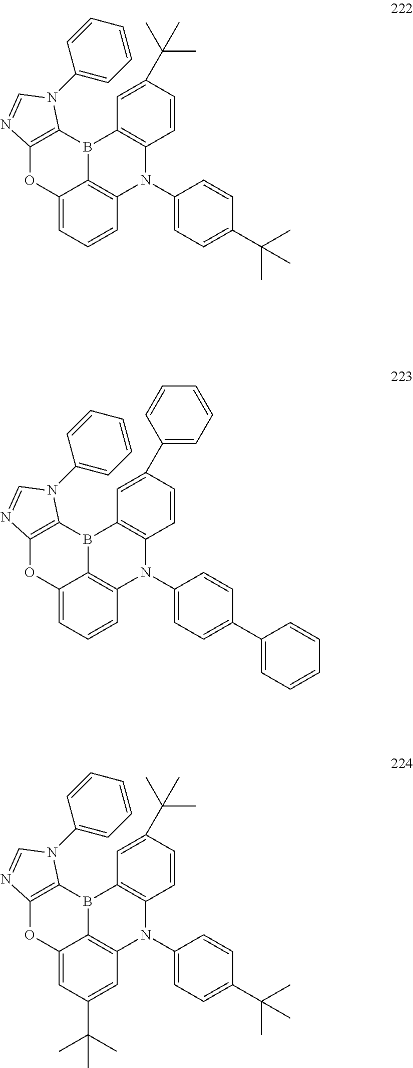 [SMILES: CC(C)(C)c1ccc(N2c3ccc(C(C)(C)C)cc3B3c4c(cccc42)Oc2ncn(-c4ccccc4)c23)cc1 ; c1ccc(-c2ccc(N3c4ccc(-c5ccccc5)cc4B4c5c(cccc53)Oc3ncn(-c5ccccc5)c34)cc2)cc1 ; CC(C)(C)c1ccc(N2c3ccc(C(C)(C)C)cc3B3c4c(cc(C(C)(C)C)cc42)Oc2ncn(-c4ccccc4)c23)cc1]

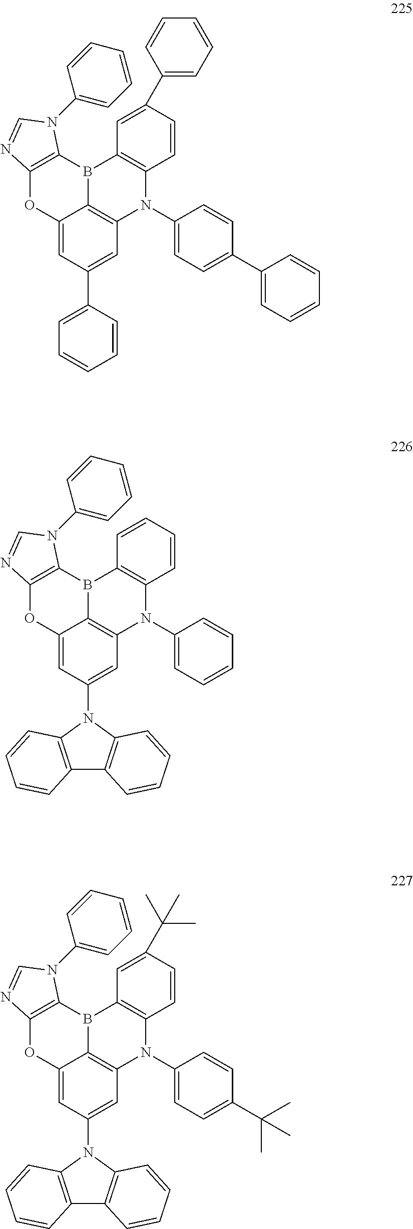 [SMILES: c1ccc(-c2ccc(N3c4ccc(-c5ccccc5)cc4B4c5c(cc(-c6ccccc6)cc53)Oc3ncn(-c5ccccc5)c34)cc2)cc1 ; c1ccc(N2c3ccccc3B3c4c(cc(-n5c6ccccc6c6ccccc65)cc42)Oc2ncn(-c4ccccc4)c23)cc1 ; CC(C)(C)c1ccc(N2c3ccc(C(C)(C)C)cc3B3c4c(cc(-n5c6ccccc6c6ccccc65)cc42)Oc2ncn(-c4ccccc4)c23)cc1]

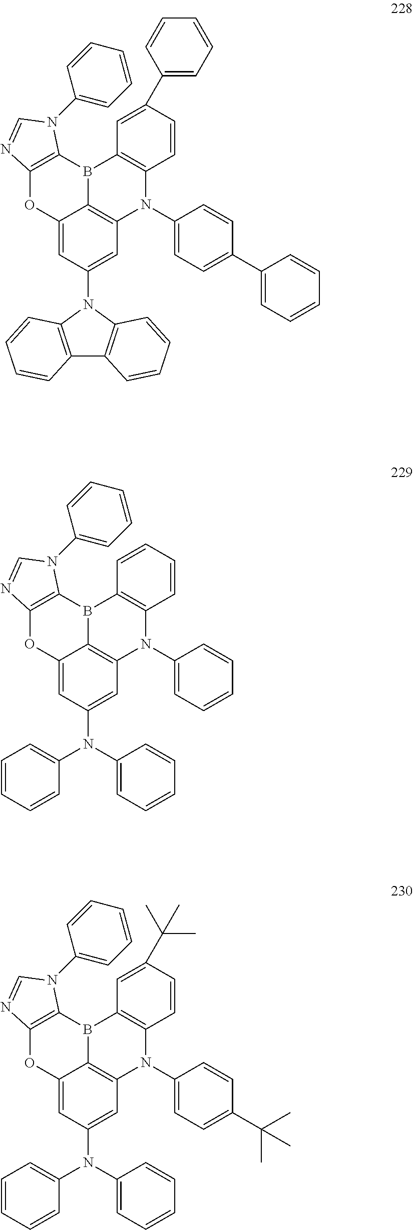 [SMILES: c1ccc(-c2ccc(N3c4ccc(-c5ccccc5)cc4B4c5c(cc(-n6c7ccccc7c7ccccc76)cc53)Oc3ncn(-c5ccccc5)c34)cc2)cc1 ; c1ccc(N(c2ccccc2)c2cc3c4c(c2)N(c2ccccc2)c2ccccc2B4c2c(ncn2-c2ccccc2)O3)cc1 ; CC(C)(C)c1ccc(N2c3ccc(C(C)(C)C)cc3B3c4c(cc(N(c5ccccc5)c5ccccc5)cc42)Oc2ncn(-c4ccccc4)c23)cc1]

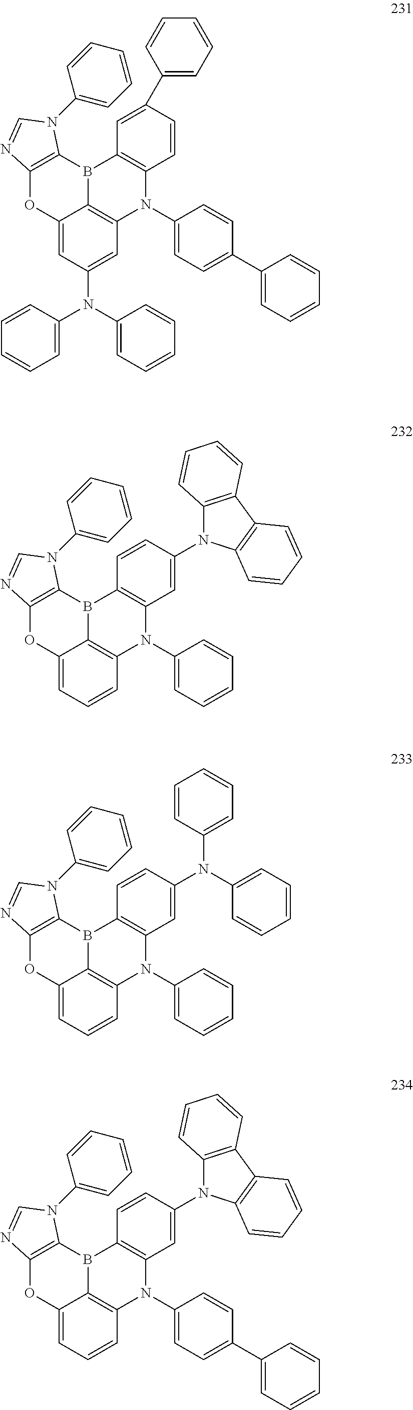 [SMILES: c1ccc(-c2ccc(N3c4ccc(-c5ccccc5)cc4B4c5c(cc(N(c6ccccc6)c6ccccc6)cc53)Oc3ncn(-c5ccccc5)c34)cc2)cc1 ; c1ccc(N2c3cc(-n4c5ccccc5c5ccccc54)ccc3B3c4c(cccc42)Oc2ncn(-c4ccccc4)c23)cc1 ; c1ccc(N(c2ccccc2)c2ccc3c(c2)N(c2ccccc2)c2cccc4c2B3c2c(ncn2-c2ccccc2)O4)cc1 ; c1ccc(-c2ccc(N3c4cc(-n5c6ccccc6c6ccccc65)ccc4B4c5c(cccc53)Oc3ncn(-c5ccccc5)c34)cc2)cc1]

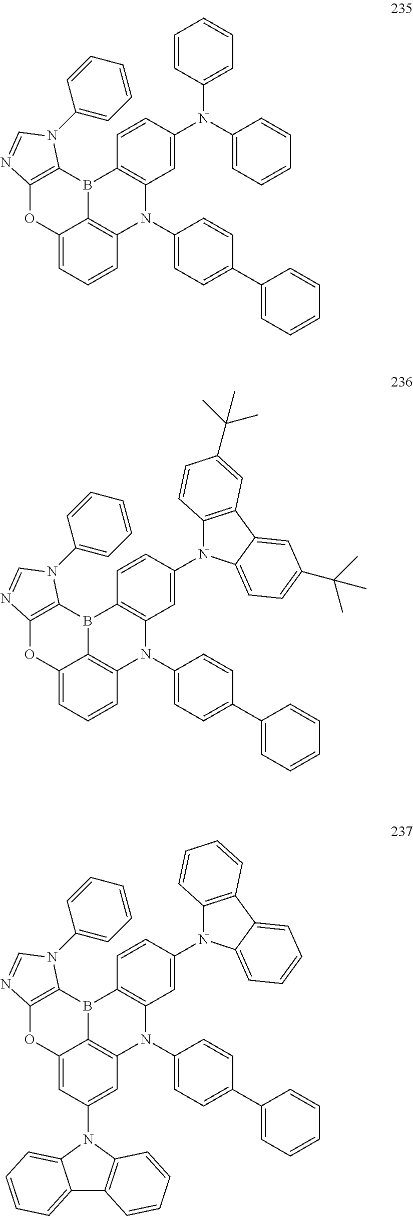 [SMILES: c1ccc(-c2ccc(N3c4cc(N(c5ccccc5)c5ccccc5)ccc4B4c5c(cccc53)Oc3ncn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(ncn1-c1ccccc1)O3 ; c1ccc(-c2ccc(N3c4cc(-n5c6ccccc6c6ccccc65)ccc4B4c5c(cc(-n6c7ccccc7c7ccccc76)cc53)Oc3ncn(-c5ccccc5)c34)cc2)cc1]

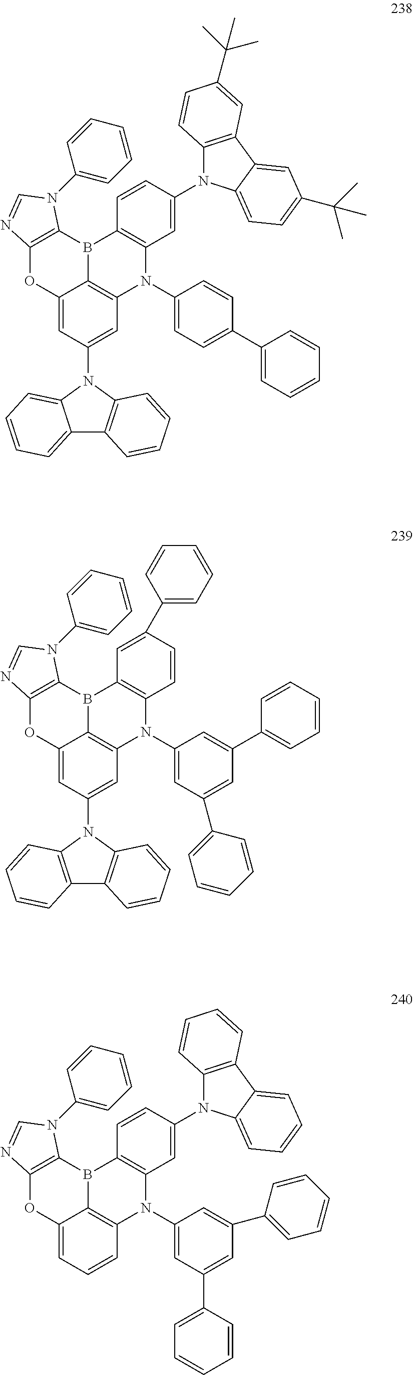 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(ncn1-c1ccccc1)O3 ; c1ccc(-c2cc(-c3ccccc3)cc(N3c4ccc(-c5ccccc5)cc4B4c5c(cc(-n6c7ccccc7c7ccccc76)cc53)Oc3ncn(-c5ccccc5)c34)c2)cc1 ; c1ccc(-c2cc(-c3ccccc3)cc(N3c4cc(-n5c6ccccc6c6ccccc65)ccc4B4c5c(cccc53)Oc3ncn(-c5ccccc5)c34)c2)cc1]

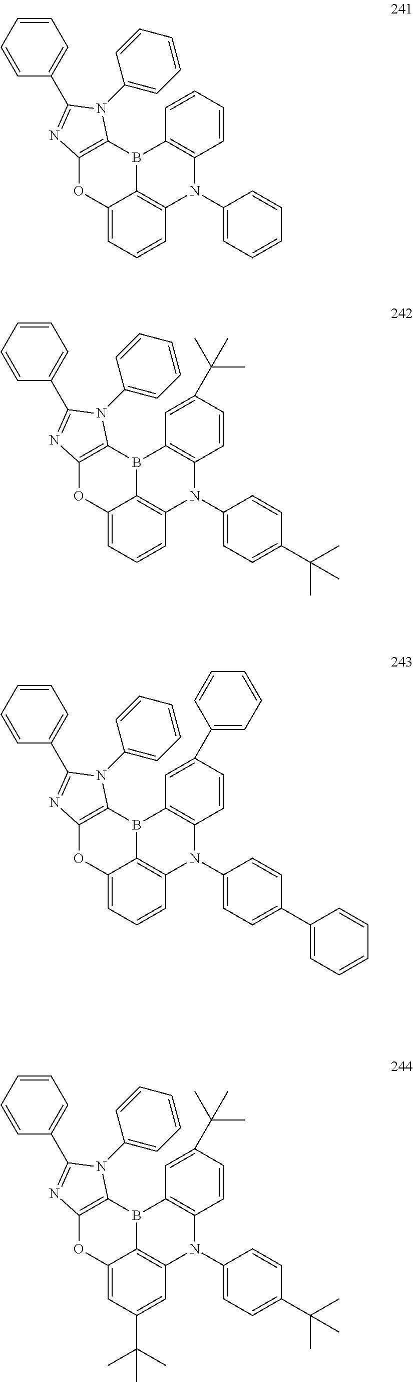 [SMILES: c1ccc(-c2nc3c(n2-c2ccccc2)B2c4ccccc4N(c4ccccc4)c4cccc(c42)O3)cc1 ; CC(C)(C)c1ccc(N2c3ccc(C(C)(C)C)cc3B3c4c(cccc42)Oc2nc(-c4ccccc4)n(-c4ccccc4)c23)cc1 ; c1ccc(-c2ccc(N3c4ccc(-c5ccccc5)cc4B4c5c(cccc53)Oc3nc(-c5ccccc5)n(-c5ccccc5)c34)cc2)cc1 ; CC(C)(C)c1ccc(N2c3ccc(C(C)(C)C)cc3B3c4c(cc(C(C)(C)C)cc42)Oc2nc(-c4ccccc4)n(-c4ccccc4)c23)cc1]

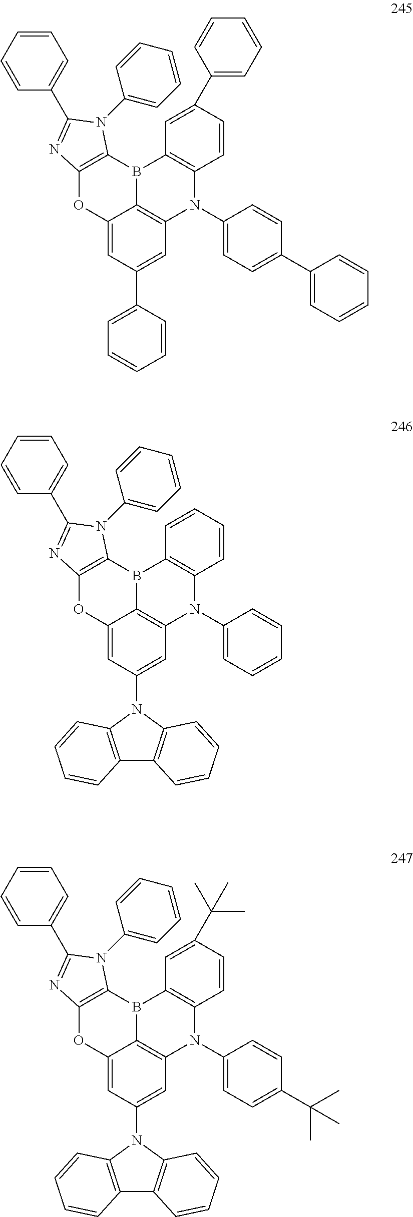 [SMILES: c1ccc(-c2ccc(N3c4ccc(-c5ccccc5)cc4B4c5c(cc(-c6ccccc6)cc53)Oc3nc(-c5ccccc5)n(-c5ccccc5)c34)cc2)cc1 ; c1ccc(-c2nc3c(n2-c2ccccc2)B2c4ccccc4N(c4ccccc4)c4cc(-n5c6ccccc6c6ccccc65)cc(c42)O3)cc1 ; CC(C)(C)c1ccc(N2c3ccc(C(C)(C)C)cc3B3c4c(cc(-n5c6ccccc6c6ccccc65)cc42)Oc2nc(-c4ccccc4)n(-c4ccccc4)c23)cc1]

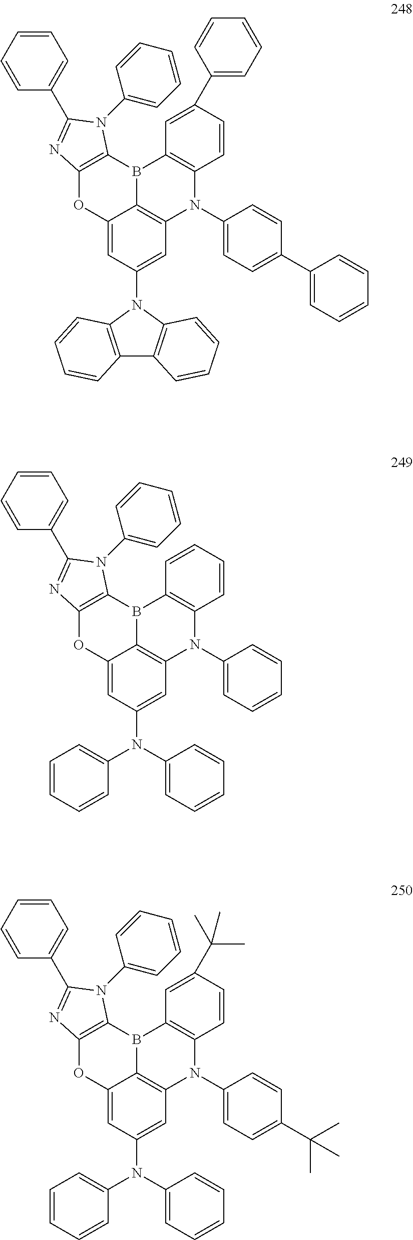 [SMILES: c1ccc(-c2ccc(N3c4ccc(-c5ccccc5)cc4B4c5c(cc(-n6c7ccccc7c7ccccc76)cc53)Oc3nc(-c5ccccc5)n(-c5ccccc5)c34)cc2)cc1 ; c1ccc(-c2nc3c(n2-c2ccccc2)B2c4ccccc4N(c4ccccc4)c4cc(N(c5ccccc5)c5ccccc5)cc(c42)O3)cc1 ; CC(C)(C)c1ccc(N2c3ccc(C(C)(C)C)cc3B3c4c(cc(N(c5ccccc5)c5ccccc5)cc42)Oc2nc(-c4ccccc4)n(-c4ccccc4)c23)cc1]

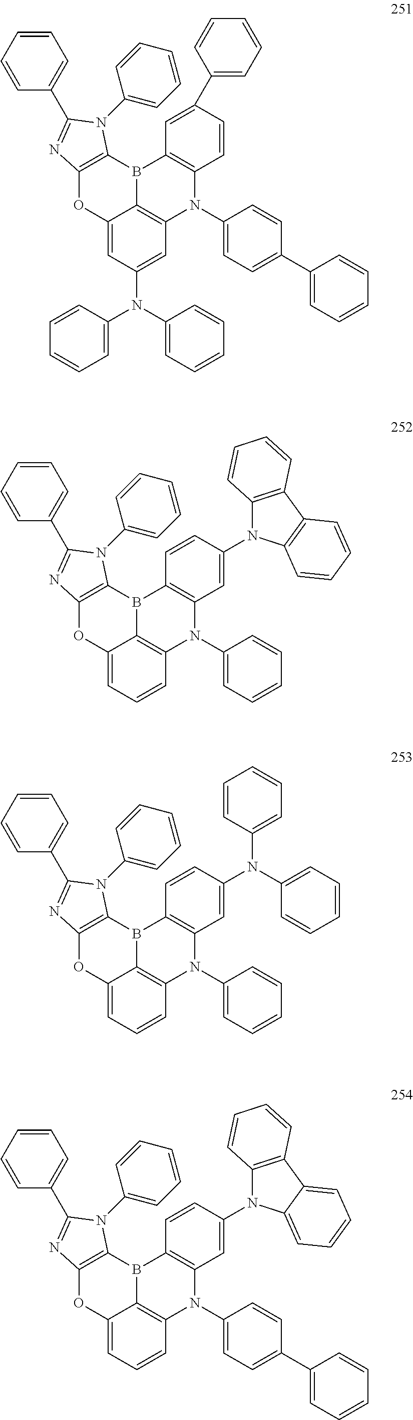 [SMILES: c1ccc(-c2ccc(N3c4ccc(-c5ccccc5)cc4B4c5c(cc(N(c6ccccc6)c6ccccc6)cc53)Oc3nc(-c5ccccc5)n(-c5ccccc5)c34)cc2)cc1 ; c1ccc(-c2nc3c(n2-c2ccccc2)B2c4ccc(-n5c6ccccc6c6ccccc65)cc4N(c4ccccc4)c4cccc(c42)O3)cc1 ; c1ccc(-c2nc3c(n2-c2ccccc2)B2c4ccc(N(c5ccccc5)c5ccccc5)cc4N(c4ccccc4)c4cccc(c42)O3)cc1 ; c1ccc(-c2ccc(N3c4cc(-n5c6ccccc6c6ccccc65)ccc4B4c5c(cccc53)Oc3nc(-c5ccccc5)n(-c5ccccc5)c34)cc2)cc1]

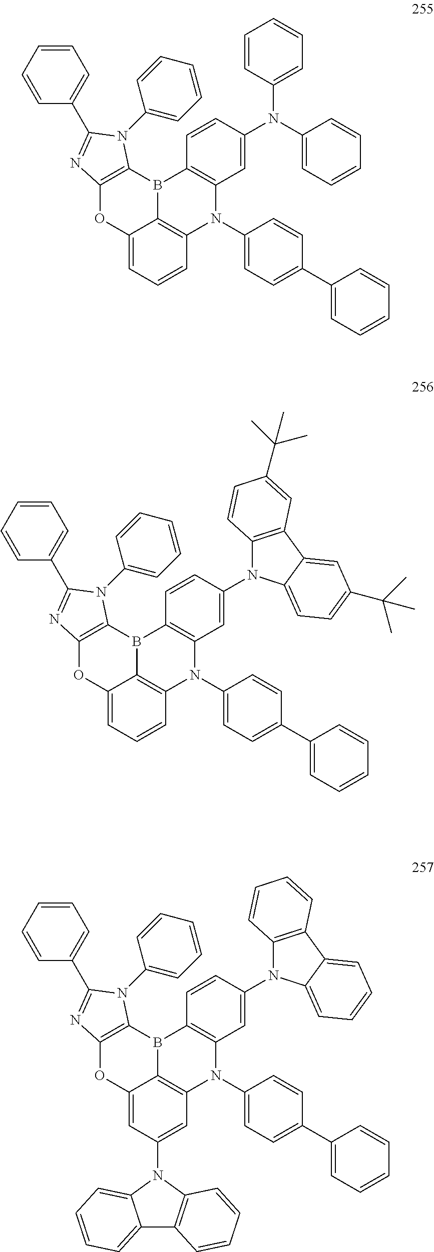 [SMILES: c1ccc(-c2ccc(N3c4cc(N(c5ccccc5)c5ccccc5)ccc4B4c5c(cccc53)Oc3nc(-c5ccccc5)n(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(nc(-c2ccccc2)n1-c1ccccc1)O3 ; c1ccc(-c2ccc(N3c4cc(-n5c6ccccc6c6ccccc65)ccc4B4c5c(cc(-n6c7ccccc7c7ccccc76)cc53)Oc3nc(-c5ccccc5)n(-c5ccccc5)c34)cc2)cc1]

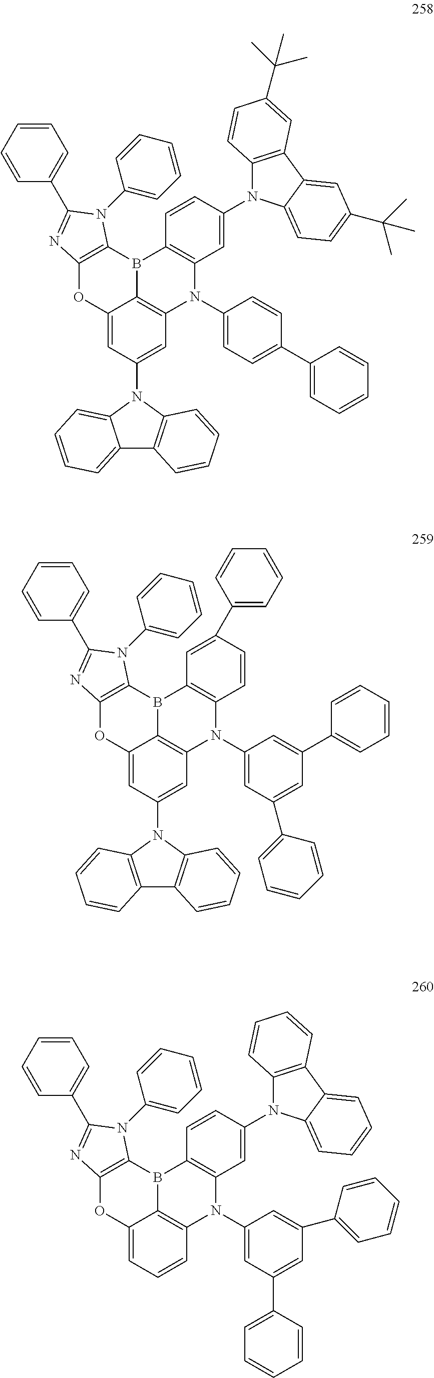 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(nc(-c2ccccc2)n1-c1ccccc1)O3 ; c1ccc(-c2cc(-c3ccccc3)cc(N3c4ccc(-c5ccccc5)cc4B4c5c(cc(-n6c7ccccc7c7ccccc76)cc53)Oc3nc(-c5ccccc5)n(-c5ccccc5)c34)c2)cc1 ; c1ccc(-c2cc(-c3ccccc3)cc(N3c4cc(-n5c6ccccc6c6ccccc65)ccc4B4c5c(cccc53)Oc3nc(-c5ccccc5)n(-c5ccccc5)c34)c2)cc1]

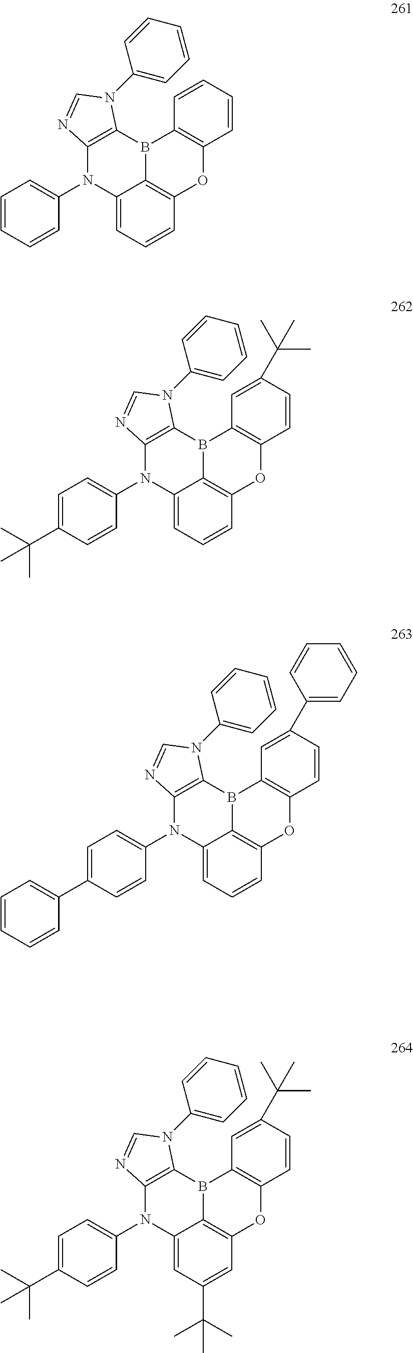

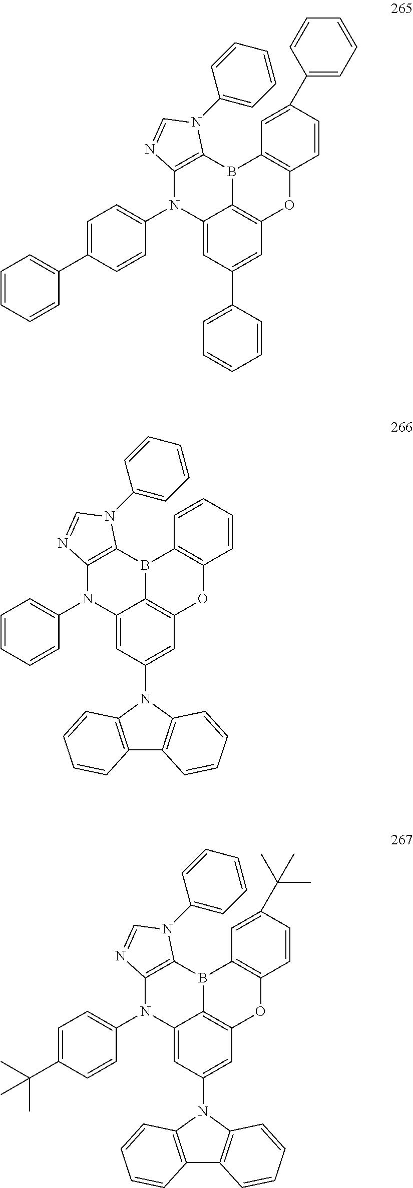 [SMILES: c1ccc(-c2ccc(N3c4cc(-c5ccccc5)cc5c4B(c4cc(-c6ccccc6)ccc4O5)c4c3ncn4-c3ccccc3)cc2)cc1 ; c1ccc(N2c3cc(-n4c5ccccc5c5ccccc54)cc4c3B(c3ccccc3O4)c3c2ncn3-c2ccccc2)cc1 ; CC(C)(C)c1ccc(N2c3cc(-n4c5ccccc5c5ccccc54)cc4c3B(c3cc(C(C)(C)C)ccc3O4)c3c2ncn3-c2ccccc2)cc1]

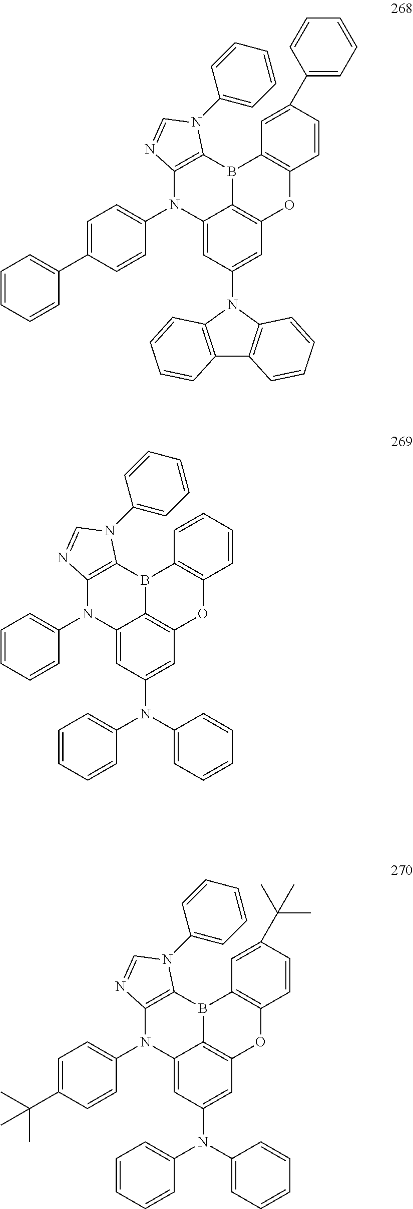 [SMILES: c1ccc(-c2ccc(N3c4cc(-n5c6ccccc6c6ccccc65)cc5c4B(c4cc(-c6ccccc6)ccc4O5)c4c3ncn4-c3ccccc3)cc2)cc1 ; c1ccc(N(c2ccccc2)c2cc3c4c(c2)N(c2ccccc2)c2ncn(-c5ccccc5)c2B4c2ccccc2O3)cc1 ; CC(C)(C)c1ccc(N2c3cc(N(c4ccccc4)c4ccccc4)cc4c3B(c3cc(C(C)(C)C)ccc3O4)c3c2ncn3-c2ccccc2)cc1]

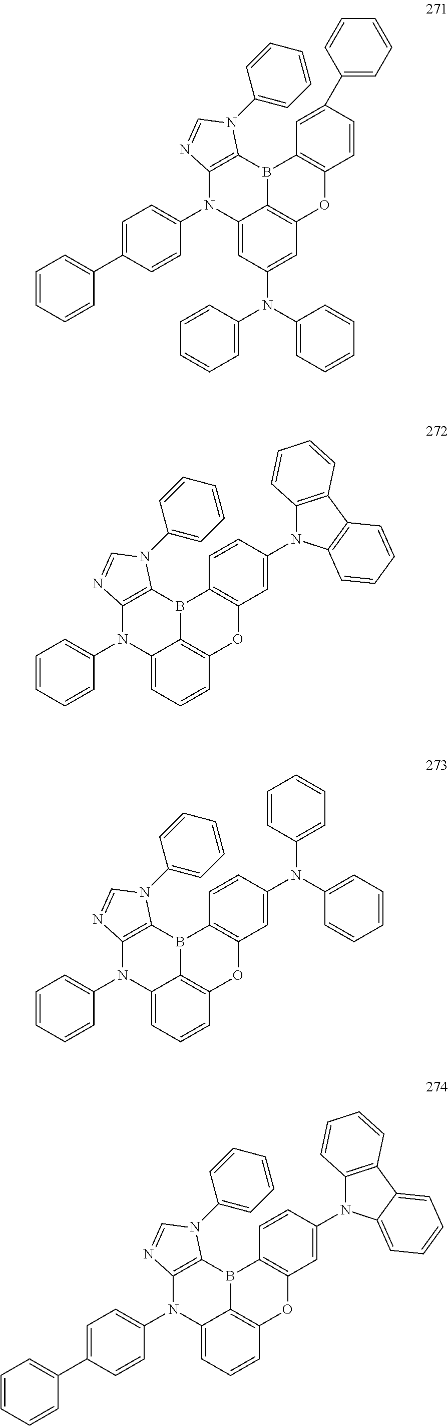 [SMILES: c1ccc(-c2ccc(N3c4cc(N(c5ccccc5)c5ccccc5)cc5c4B(c4cc(-c6ccccc6)ccc4O5)c4c3ncn4-c3ccccc3)cc2)cc1 ; c1ccc(N2c3cccc4c3B(c3ccc(-n5c6ccccc6c6ccccc65)cc3O4)c3c2ncn3-c2ccccc2)cc1 ; c1ccc(N(c2ccccc2)c2ccc3c(c2)Oc2cccc4c2B3c2c(ncn2-c2ccccc2)N4c2ccccc2)cc1 ; c1ccc(-c2ccc(N3c4cccc5c4B(c4ccc(-n6c7ccccc7c7ccccc76)cc4O5)c4c3ncn4-c3ccccc3)cc2)cc1]

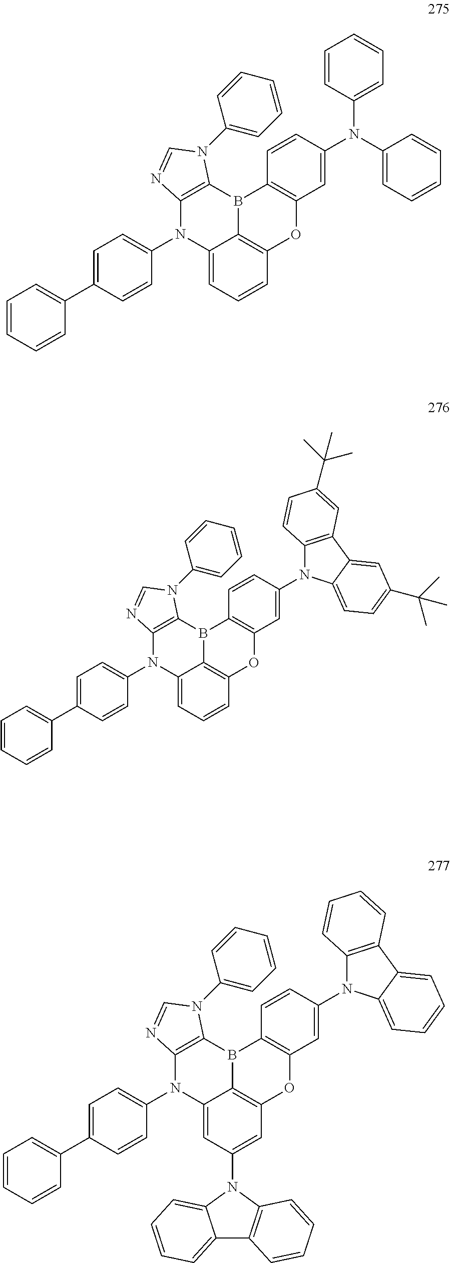 [SMILES: c1ccc(-c2ccc(N3c4cccc5c4B(c4ccc(N(c6ccccc6)c6ccccc6)cc4O5)c4c3ncn4-c3ccccc3)cc2)cc1 ; CC(C)(C)c1ccc2c(c1)c1cc(C(C)(C)C)ccc1n2-c1ccc2c(c1)Oc1cccc3c1B2c1c(ncn1-c1ccccc1)N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4c3ncn4-c3ccccc3)cc2)cc1]

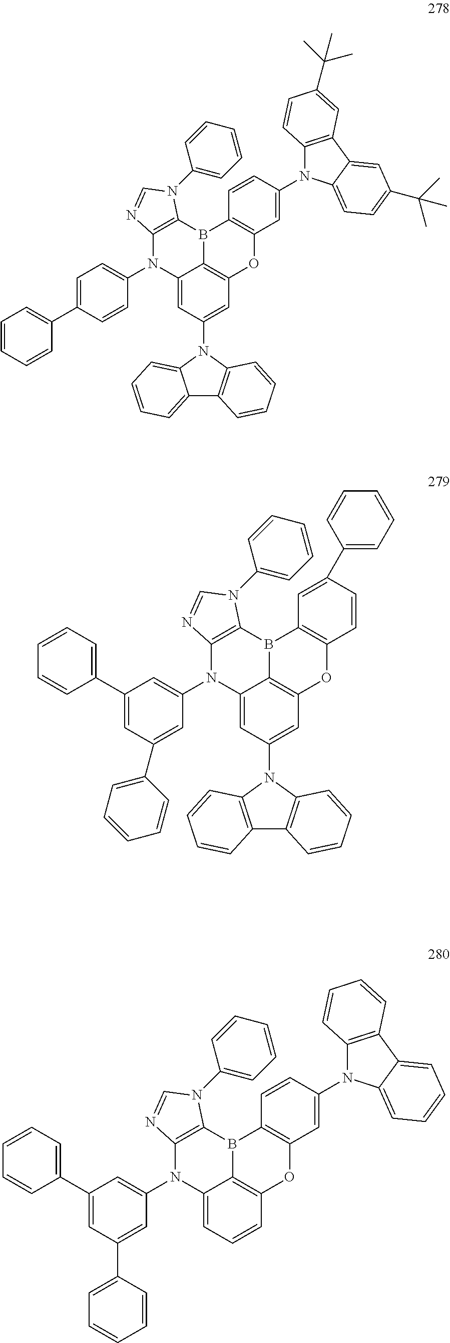 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(ncn1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3ncn4-c3ccccc3)c2)cc1 ; c1ccc(-c2cc(-c3ccccc3)cc(N3c4cccc5c4B(c4ccc(-n6c7ccccc7c7ccccc76)cc4O5)c4c3ncn4-c3ccccc3)c2)cc1]

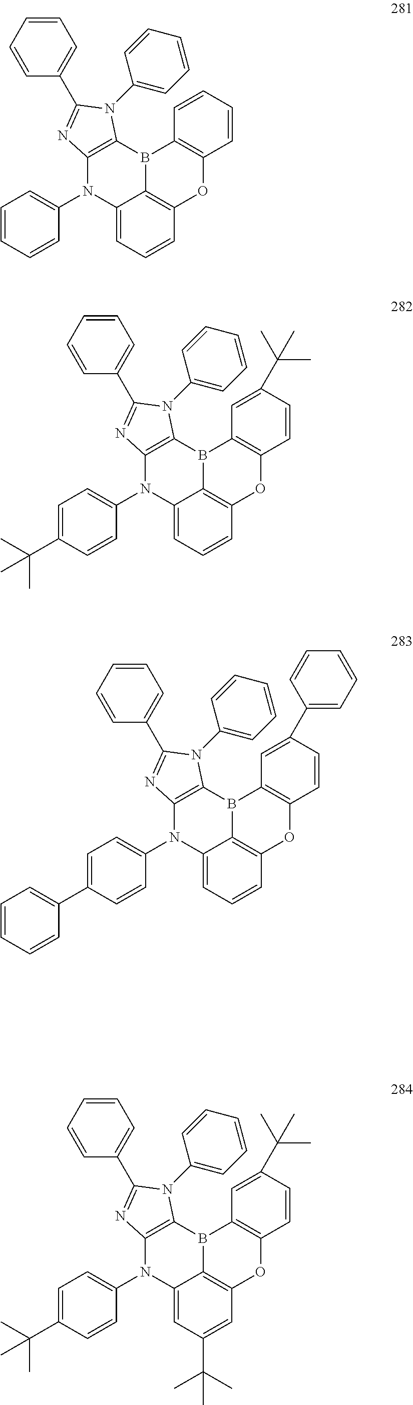

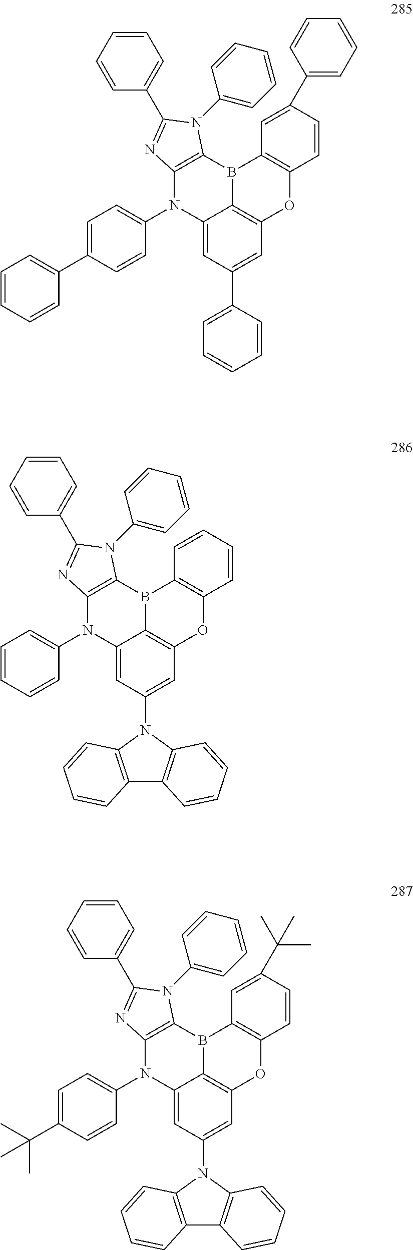 [SMILES: c1ccc(-c2ccc(N3c4cc(-c5ccccc5)cc5c4B(c4cc(-c6ccccc6)ccc4O5)c4c3nc(-c3ccccc3)n4-c3ccccc3)cc2)cc1 ; c1ccc(-c2nc3c(n2-c2ccccc2)B2c4ccccc4Oc4cc(-n5c6ccccc6c6ccccc65)cc(c42)N3c2ccccc2)cc1 ; CC(C)(C)c1ccc(N2c3cc(-n4c5ccccc5c5ccccc54)cc4c3B(c3cc(C(C)(C)C)ccc3O4)c3c2nc(-c2ccccc2)n3-c2ccccc2)cc1]

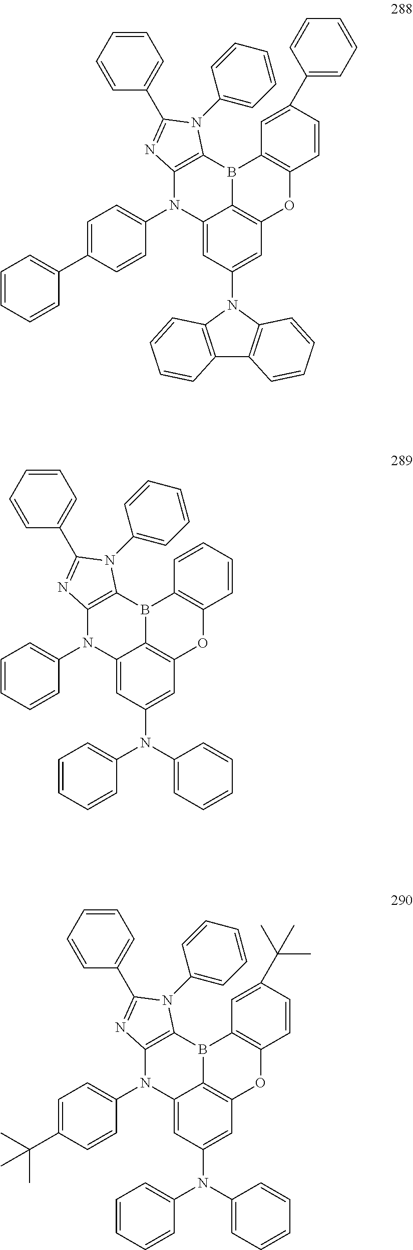 [SMILES: c1ccc(-c2ccc(N3c4cc(-n5c6ccccc6c6ccccc65)cc5c4B(c4cc(-c6ccccc6)ccc4O5)c4c3nc(-c3ccccc3)n4-c3ccccc3)cc2)cc1 ; c1ccc(-c2nc3c(n2-c2ccccc2)B2c4ccccc4Oc4cc(N(c5ccccc5)c5ccccc5)cc(c42)N3c2ccccc2)cc1 ; CC(C)(C)c1ccc(N2c3cc(N(c4ccccc4)c4ccccc4)cc4c3B(c3cc(C(C)(C)C)ccc3O4)c3c2nc(-c2ccccc2)n3-c2ccccc2)cc1]

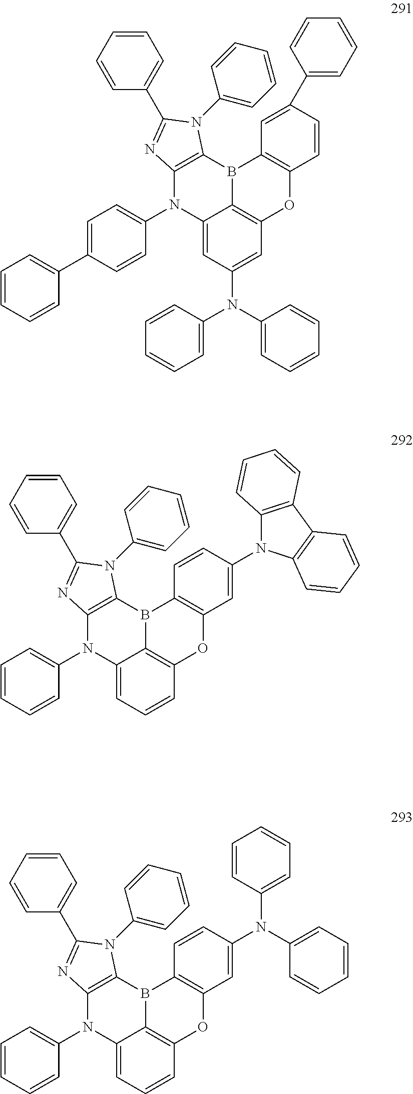 [SMILES: c1ccc(-c2ccc(N3c4cc(N(c5ccccc5)c5ccccc5)cc5c4B(c4cc(-c6ccccc6)ccc4O5)c4c3nc(-c3ccccc3)n4-c3ccccc3)cc2)cc1 ; c1ccc(-c2nc3c(n2-c2ccccc2)B2c4ccc(-n5c6ccccc6c6ccccc65)cc4Oc4cccc(c42)N3c2ccccc2)cc1 ; c1ccc(-c2nc3c(n2-c2ccccc2)B2c4ccc(N(c5ccccc5)c5ccccc5)cc4Oc4cccc(c42)N3c2ccccc2)cc1]

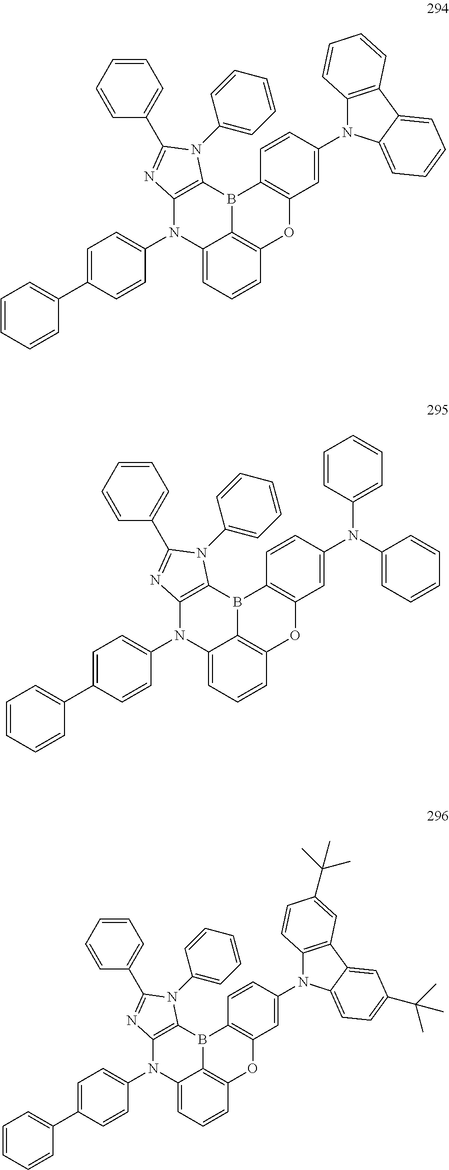 [SMILES: c1ccc(-c2ccc(N3c4cccc5c4B(c4ccc(-n6c7ccccc7c7ccccc76)cc4O5)c4c3nc(-c3ccccc3)n4-c3ccccc3)cc2)cc1 ; c1ccc(-c2ccc(N3c4cccc5c4B(c4ccc(N(c6ccccc6)c6ccccc6)cc4O5)c4c3nc(-c3ccccc3)n4-c3ccccc3)cc2)cc1 ; CC(C)(C)c1ccc2c(c1)c1cc(C(C)(C)C)ccc1n2-c1ccc2c(c1)Oc1cccc3c1B2c1c(nc(-c2ccccc2)n1-c1ccccc1)N3c1ccc(-c2ccccc2)cc1]

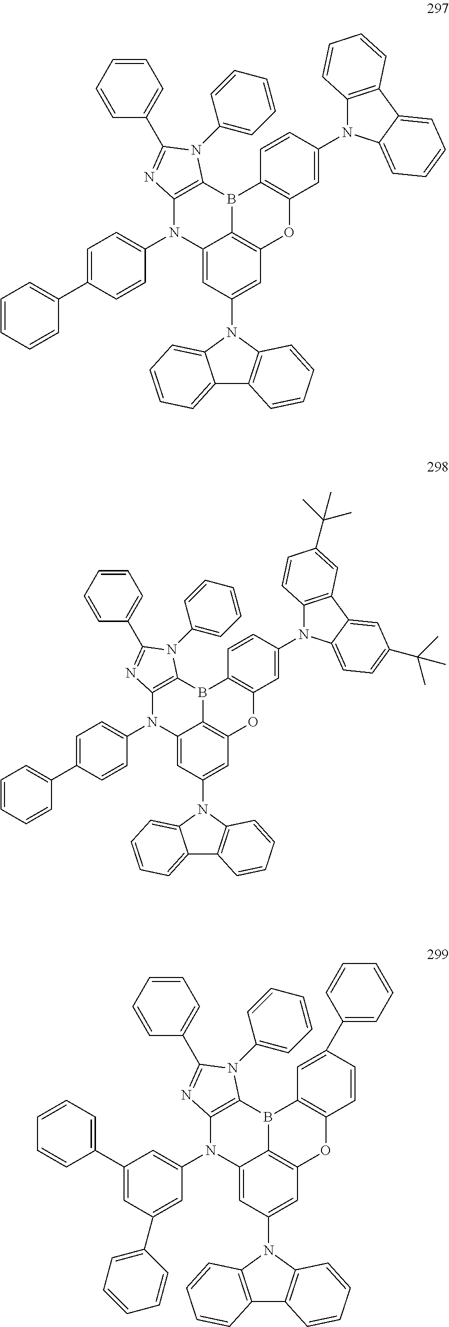 [SMILES: c1ccc(-c2ccc(N3c4cc(-n5c6ccccc6c6ccccc65)cc5c4B(c4ccc(-n6c7ccccc7c7ccccc76)cc4O5)c4c3nc(-c3ccccc3)n4-c3ccccc3)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1c(nc(-c2ccccc2)n1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3nc(-c3ccccc3)n4-c3ccccc3)c2)cc1]

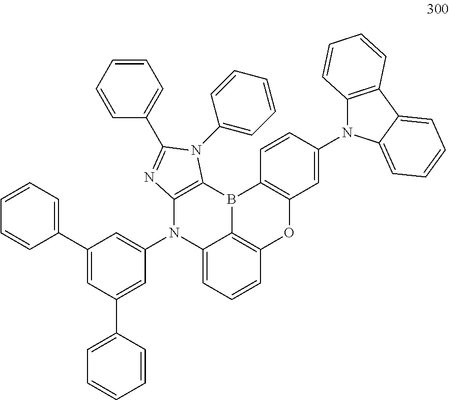 [SMILES: c1ccc(-c2cc(-c3ccccc3)cc(N3c4cccc5c4B(c4ccc(-n6c7ccccc7c7ccccc76)cc4O5)c4c3nc(-c3ccccc3)n4-c3ccccc3)c2)cc1]

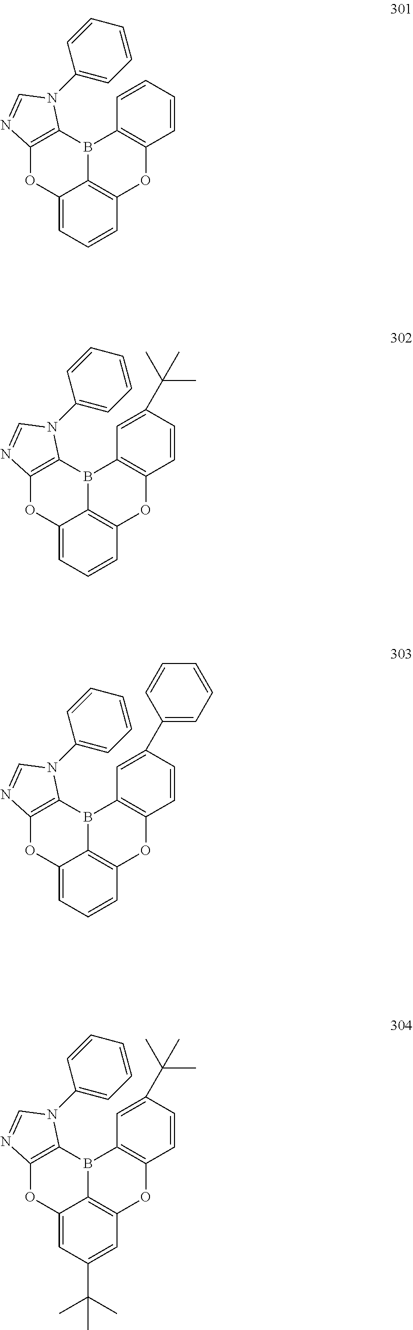 [SMILES: c1ccc(-n2cnc3c2B2c4ccccc4Oc4cccc(c42)O3)cc1 ; CC(C)(C)c1ccc2c(c1)B1c3c(cccc3Oc3ncn(-c4ccccc4)c31)O2 ; c1ccc(-c2ccc3c(c2)B2c4c(cccc4Oc4ncn(-c5ccccc5)c42)O3)cc1 ; CC(C)(C)c1cc2c3c(c1)Oc1ncn(-c4ccccc4)c1B3c1cc(C(C)(C)C)ccc1O2]

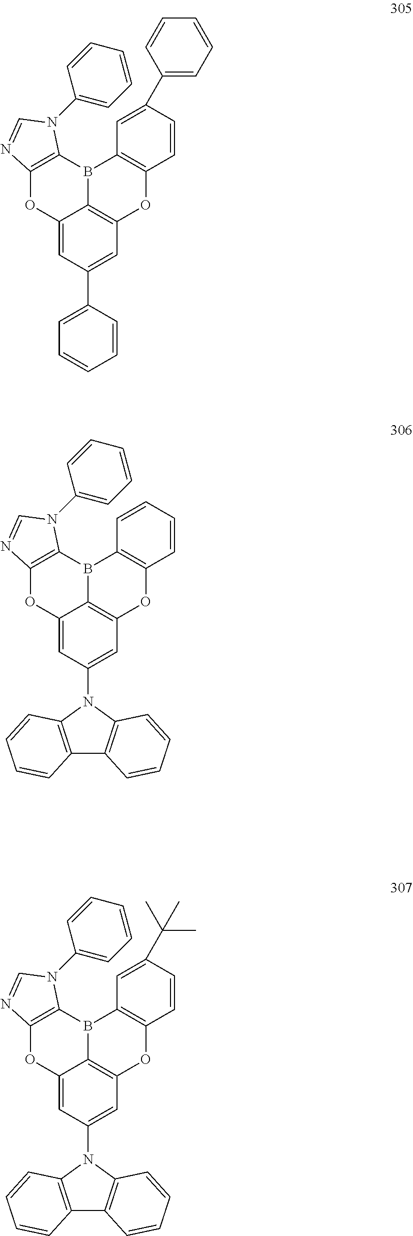 [SMILES: c1ccc(-c2cc3c4c(c2)Oc2ncn(-c5ccccc5)c2B4c2cc(-c4ccccc4)ccc2O3)cc1 ; c1ccc(-n2cnc3c2B2c4ccccc4Oc4cc(-n5c6ccccc6c6ccccc65)cc(c42)O3)cc1 ; CC(C)(C)c1ccc2c(c1)B1c3c(cc(-n4c5ccccc5c5ccccc54)cc3Oc3ncn(-c4ccccc4)c31)O2]

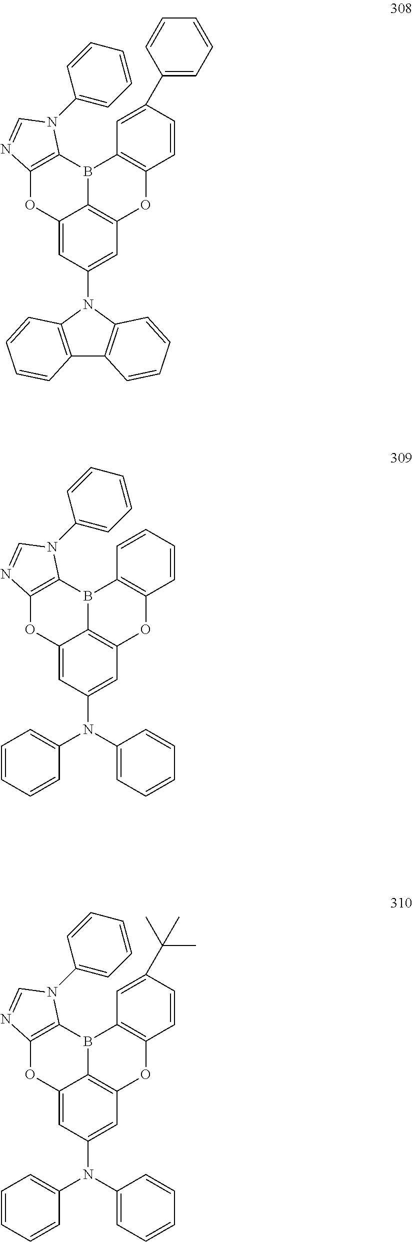 [SMILES: c1ccc(-c2ccc3c(c2)B2c4c(cc(-n5c6ccccc6c6ccccc65)cc4Oc4ncn(-c5ccccc5)c42)O3)cc1 ; c1ccc(N(c2ccccc2)c2cc3c4c(c2)Oc2ncn(-c5ccccc5)c2B4c2ccccc2O3)cc1 ; CC(C)(C)c1ccc2c(c1)B1c3c(cc(N(c4ccccc4)c4ccccc4)cc3Oc3ncn(-c4ccccc4)c31)O2]

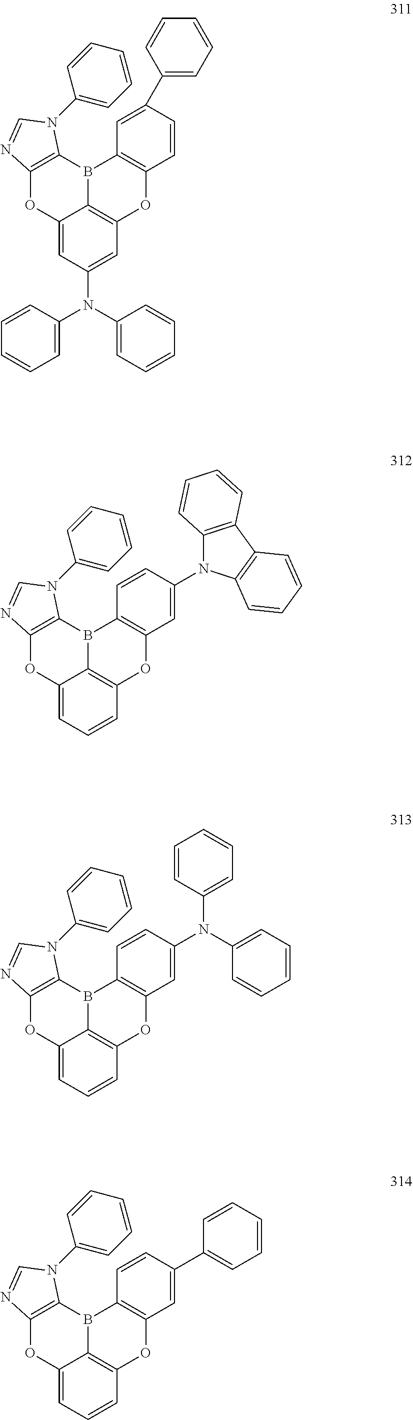 [SMILES: c1ccc(-c2ccc3c(c2)B2c4c(cc(N(c5ccccc5)c5ccccc5)cc4Oc4ncn(-c5ccccc5)c42)O3)cc1 ; c1ccc(-n2cnc3c2B2c4ccc(-n5c6ccccc6c6ccccc65)cc4Oc4cccc(c42)O3)cc1 ; c1ccc(N(c2ccccc2)c2ccc3c(c2)Oc2cccc4c2B3c2c(ncn2-c2ccccc2)O4)cc1 ; c1ccc(-c2ccc3c(c2)Oc2cccc4c2B3c2c(ncn2-c2ccccc2)O4)cc1]

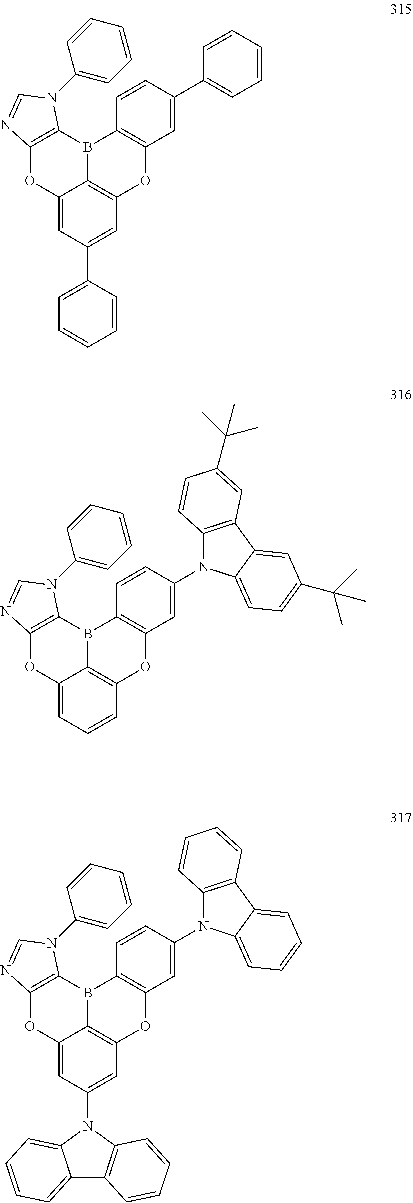 [SMILES: c1ccc(-c2ccc3c(c2)Oc2cc(-c4ccccc4)cc4c2B3c2c(ncn2-c2ccccc2)O4)cc1 ; CC(C)(C)c1ccc2c(c1)c1cc(C(C)(C)C)ccc1n2-c1ccc2c(c1)Oc1cccc3c1B2c1c(ncn1-c1ccccc1)O3 ; c1ccc(-n2cnc3c2B2c4ccc(-n5c6ccccc6c6ccccc65)cc4Oc4cc(-n5c6ccccc6c6ccccc65)cc(c42)O3)cc1]

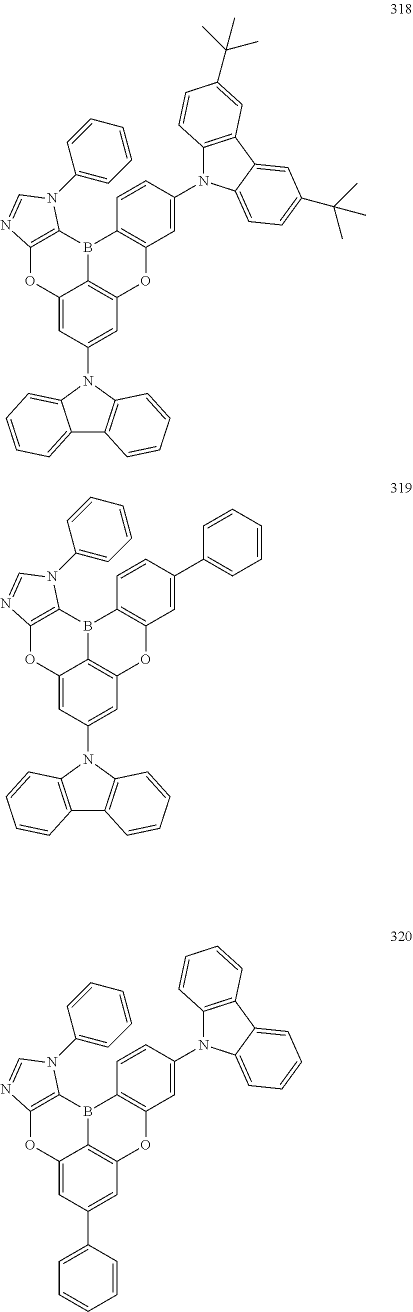 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(ncn1-c1ccccc1)O3 ; c1ccc(-c2ccc3c(c2)Oc2cc(-n4c5ccccc5c5ccccc54)cc4c2B3c2c(ncn2-c2ccccc2)O4)cc1 ; c1ccc(-c2cc3c4c(c2)Oc2ncn(-c5ccccc5)c2B4c2ccc(-n4c5ccccc5c5ccccc54)cc2O3)cc1]

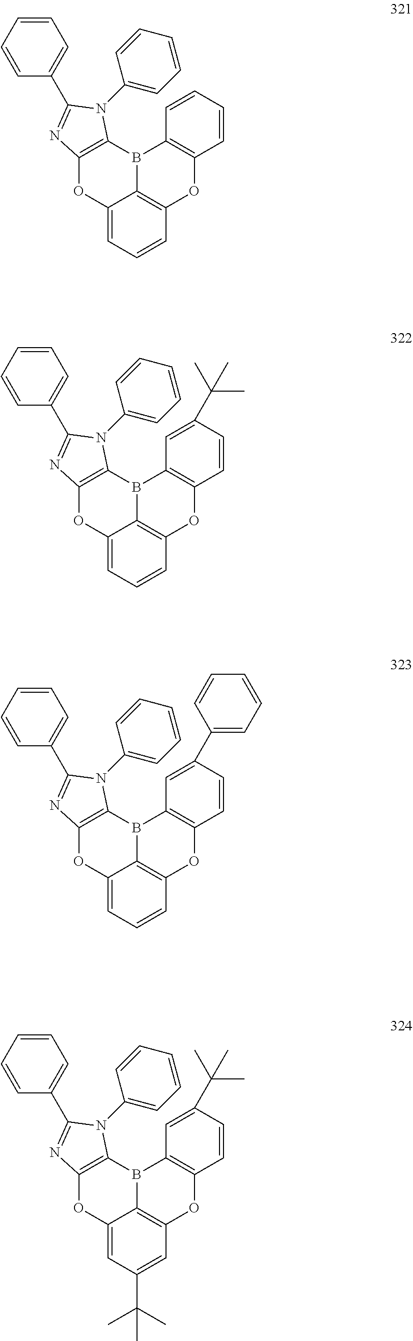 [SMILES: c1ccc(-c2nc3c(n2-c2ccccc2)B2c4ccccc4Oc4cccc(c42)O3)cc1 ; CC(C)(C)c1ccc2c(c1)B1c3c(cccc3Oc3nc(-c4ccccc4)n(-c4ccccc4)c31)O2 ; c1ccc(-c2ccc3c(c2)B2c4c(cccc4Oc4nc(-c5ccccc5)n(-c5ccccc5)c42)O3)cc1 ; CC(C)(C)c1cc2c3c(c1)Oc1nc(-c4ccccc4)n(-c4ccccc4)c1B3c1cc(C(C)(C)C)ccc1O2]

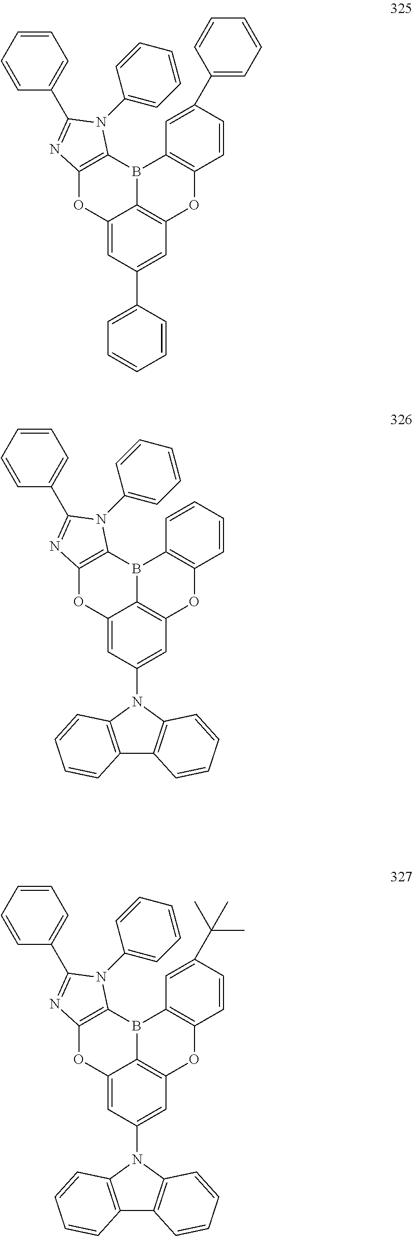 [SMILES: c1ccc(-c2cc3c4c(c2)Oc2nc(-c5ccccc5)n(-c5ccccc5)c2B4c2cc(-c4ccccc4)ccc2O3)cc1 ; c1ccc(-c2nc3c(n2-c2ccccc2)B2c4ccccc4Oc4cc(-n5c6ccccc6c6ccccc65)cc(c42)O3)cc1 ; CC(C)(C)c1ccc2c(c1)B1c3c(cc(-n4c5ccccc5c5ccccc54)cc3Oc3nc(-c4ccccc4)n(-c4ccccc4)c31)O2]

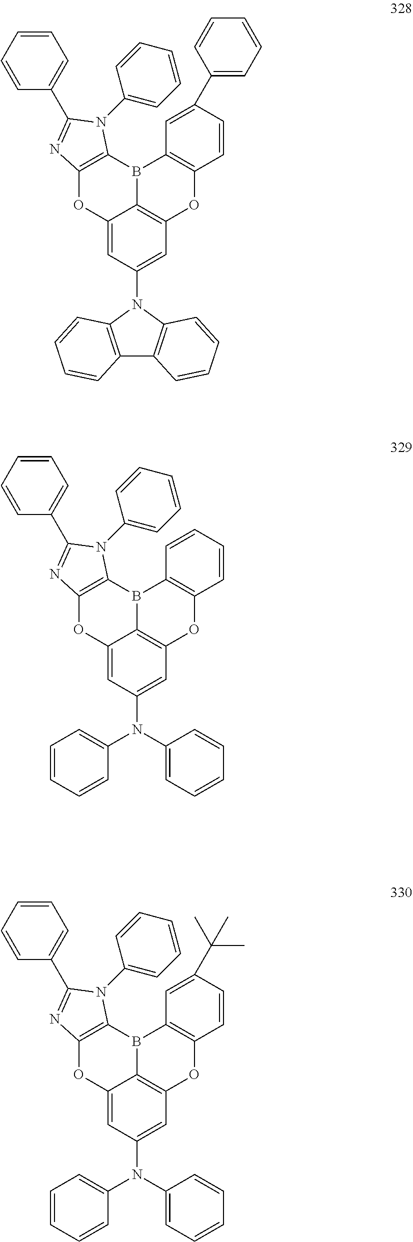 [SMILES: c1ccc(-c2ccc3c(c2)B2c4c(cc(-n5c6ccccc6c6ccccc65)cc4Oc4nc(-c5ccccc5)n(-c5ccccc5)c42)O3)cc1 ; c1ccc(-c2nc3c(n2-c2ccccc2)B2c4ccccc4Oc4cc(N(c5ccccc5)c5ccccc5)cc(c42)O3)cc1 ; CC(C)(C)c1ccc2c(c1)B1c3c(cc(N(c4ccccc4)c4ccccc4)cc3Oc3nc(-c4ccccc4)n(-c4ccccc4)c31)O2]

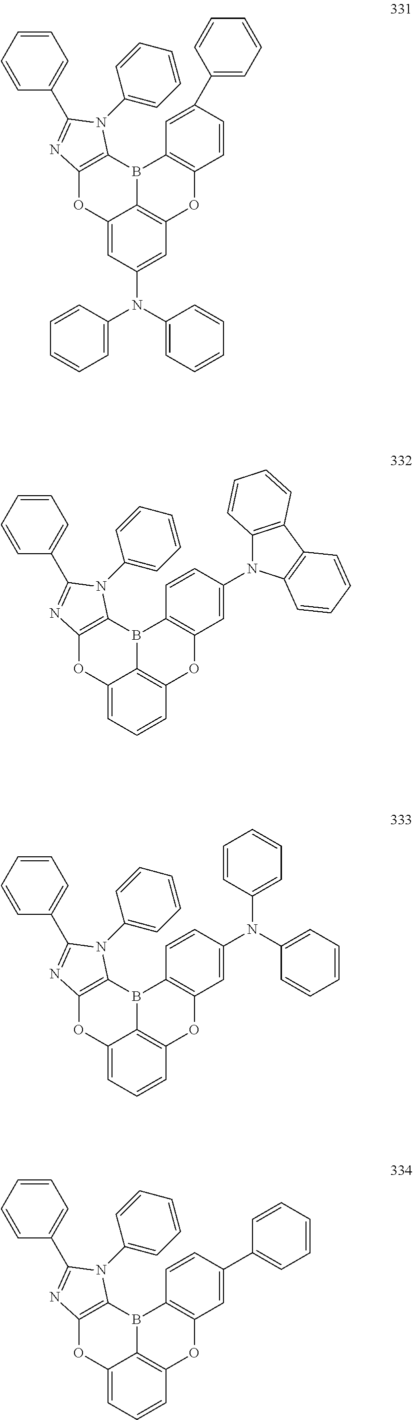 [SMILES: c1ccc(-c2ccc3c(c2)B2c4c(cc(N(c5ccccc5)c5ccccc5)cc4Oc4nc(-c5ccccc5)n(-c5ccccc5)c42)O3)cc1 ; c1ccc(-c2nc3c(n2-c2ccccc2)B2c4ccc(-n5c6ccccc6c6ccccc65)cc4Oc4cccc(c42)O3)cc1 ; c1ccc(-c2nc3c(n2-c2ccccc2)B2c4ccc(N(c5ccccc5)c5ccccc5)cc4Oc4cccc(c42)O3)cc1 ; c1ccc(-c2ccc3c(c2)Oc2cccc4c2B3c2c(nc(-c3ccccc3)n2-c2ccccc2)O4)cc1]

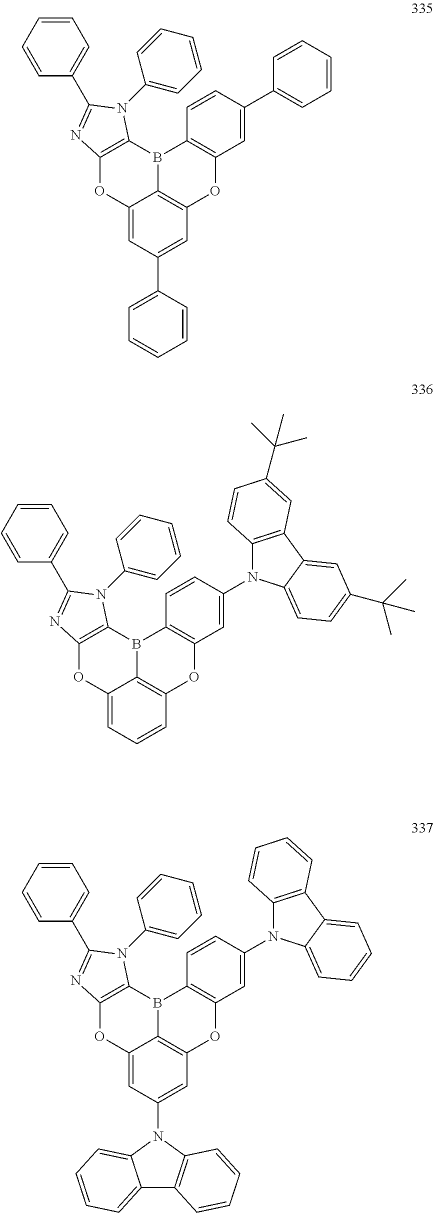 [SMILES: c1ccc(-c2ccc3c(c2)Oc2cc(-c4ccccc4)cc4c2B3c2c(nc(-c3ccccc3)n2-c2ccccc2)O4)cc1 ; CC(C)(C)c1ccc2c(c1)c1cc(C(C)(C)C)ccc1n2-c1ccc2c(c1)Oc1cccc3c1B2c1c(nc(-c2ccccc2)n1-c1ccccc1)O3 ; c1ccc(-c2nc3c(n2-c2ccccc2)B2c4ccc(-n5c6ccccc6c6ccccc65)cc4Oc4cc(-n5c6ccccc6c6ccccc65)cc(c42)O3)cc1]

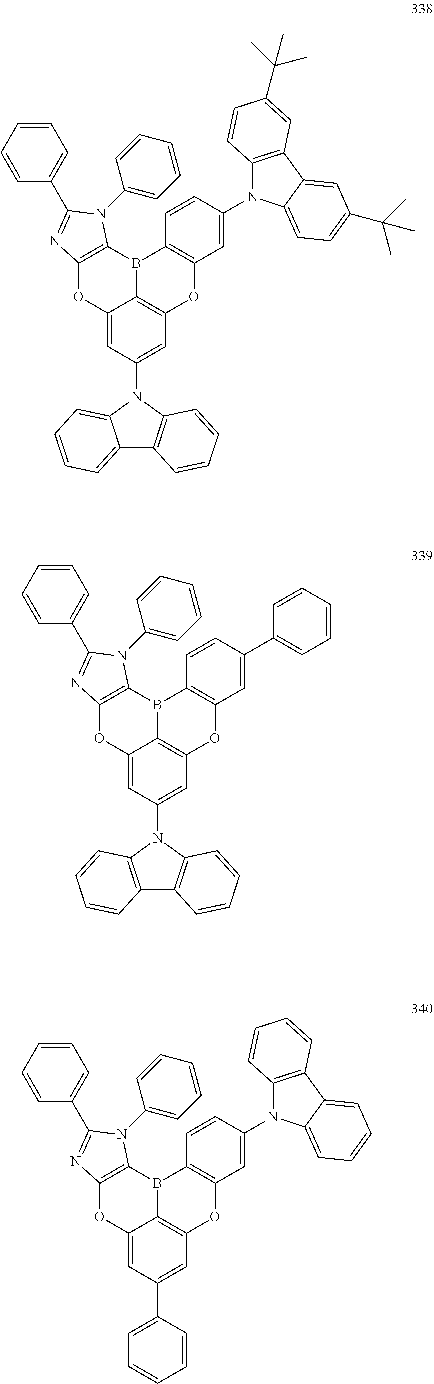 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(nc(-c2ccccc2)n1-c1ccccc1)O3 ; c1ccc(-c2ccc3c(c2)Oc2cc(-n4c5ccccc5c5ccccc54)cc4c2B3c2c(nc(-c3ccccc3)n2-c2ccccc2)O4)cc1 ; c1ccc(-c2cc3c4c(c2)Oc2nc(-c5ccccc5)n(-c5ccccc5)c2B4c2ccc(-n4c5ccccc5c5ccccc54)cc2O3)cc1]

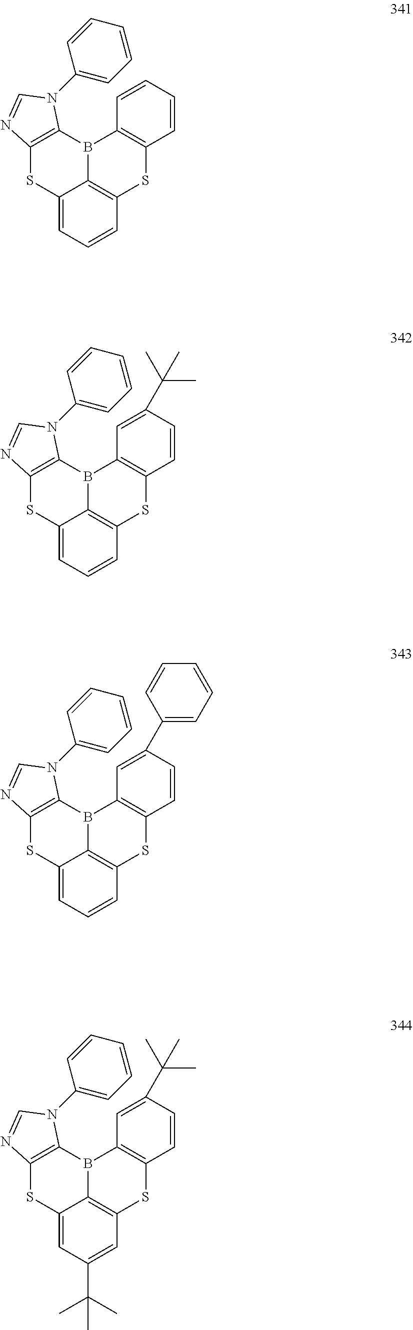 [SMILES: c1ccc(-n2cnc3c2B2c4ccccc4Sc4cccc(c42)S3)cc1 ; CC(C)(C)c1ccc2c(c1)B1c3c(cccc3Sc3ncn(-c4ccccc4)c31)S2 ; c1ccc(-c2ccc3c(c2)B2c4c(cccc4Sc4ncn(-c5ccccc5)c42)S3)cc1 ; CC(C)(C)c1cc2c3c(c1)Sc1ncn(-c4ccccc4)c1B3c1cc(C(C)(C)C)ccc1S2]

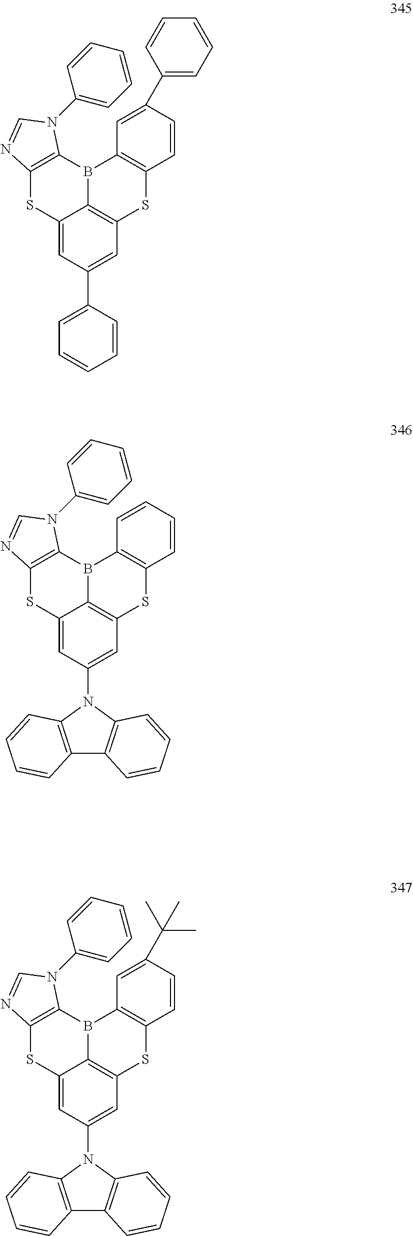 [SMILES: c1ccc(-c2cc3c4c(c2)Sc2ncn(-c5ccccc5)c2B4c2cc(-c4ccccc4)ccc2S3)cc1 ; c1ccc(-n2cnc3c2B2c4ccccc4Sc4cc(-n5c6ccccc6c6ccccc65)cc(c42)S3)cc1 ; CC(C)(C)c1ccc2c(c1)B1c3c(cc(-n4c5ccccc5c5ccccc54)cc3Sc3ncn(-c4ccccc4)c31)S2]

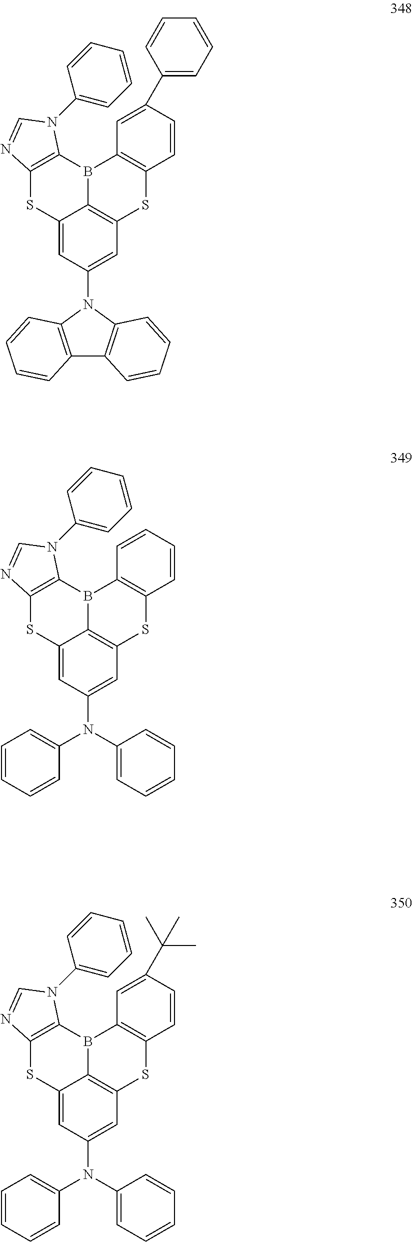 [SMILES: c1ccc(-c2ccc3c(c2)B2c4c(cc(-n5c6ccccc6c6ccccc65)cc4Sc4ncn(-c5ccccc5)c42)S3)cc1 ; c1ccc(N(c2ccccc2)c2cc3c4c(c2)Sc2ncn(-c5ccccc5)c2B4c2ccccc2S3)cc1 ; CC(C)(C)c1ccc2c(c1)B1c3c(cc(N(c4ccccc4)c4ccccc4)cc3Sc3ncn(-c4ccccc4)c31)S2]

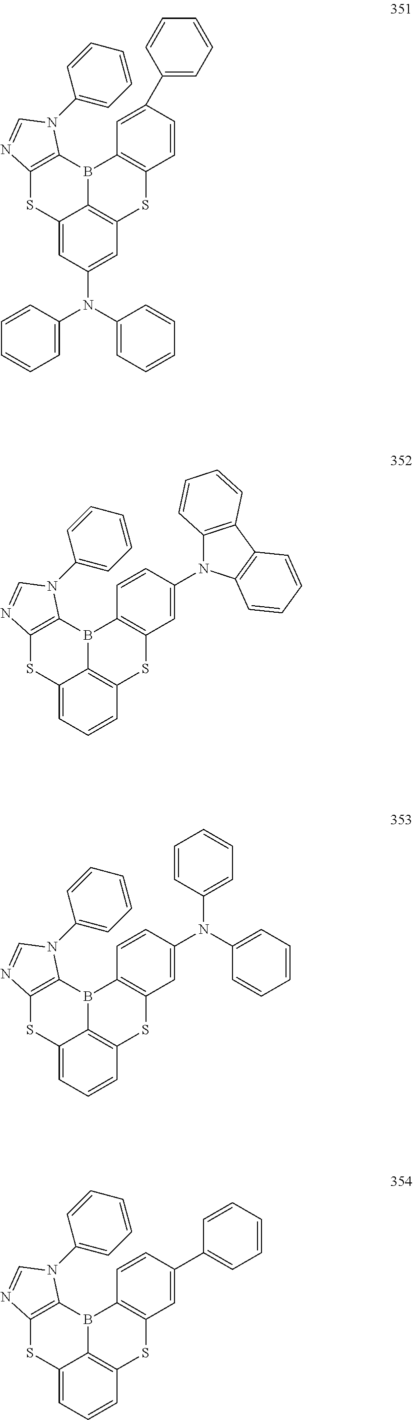 [SMILES: c1ccc(-c2ccc3c(c2)B2c4c(cc(N(c5ccccc5)c5ccccc5)cc4Sc4ncn(-c5ccccc5)c42)S3)cc1 ; c1ccc(-n2cnc3c2B2c4ccc(-n5c6ccccc6c6ccccc65)cc4Sc4cccc(c42)S3)cc1 ; c1ccc(N(c2ccccc2)c2ccc3c(c2)Sc2cccc4c2B3c2c(ncn2-c2ccccc2)S4)cc1 ; c1ccc(-c2ccc3c(c2)Sc2cccc4c2B3c2c(ncn2-c2ccccc2)S4)cc1]

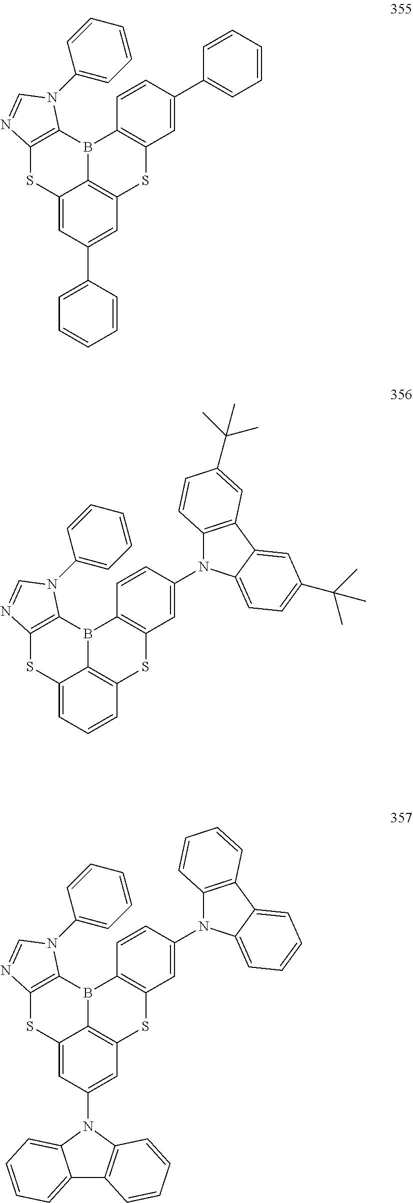 [SMILES: c1ccc(-c2ccc3c(c2)Sc2cc(-c4ccccc4)cc4c2B3c2c(ncn2-c2ccccc2)S4)cc1 ; CC(C)(C)c1ccc2c(c1)c1cc(C(C)(C)C)ccc1n2-c1ccc2c(c1)Sc1cccc3c1B2c1c(ncn1-c1ccccc1)S3 ; c1ccc(-n2cnc3c2B2c4ccc(-n5c6ccccc6c6ccccc65)cc4Sc4cc(-n5c6ccccc6c6ccccc65)cc(c42)S3)cc1]

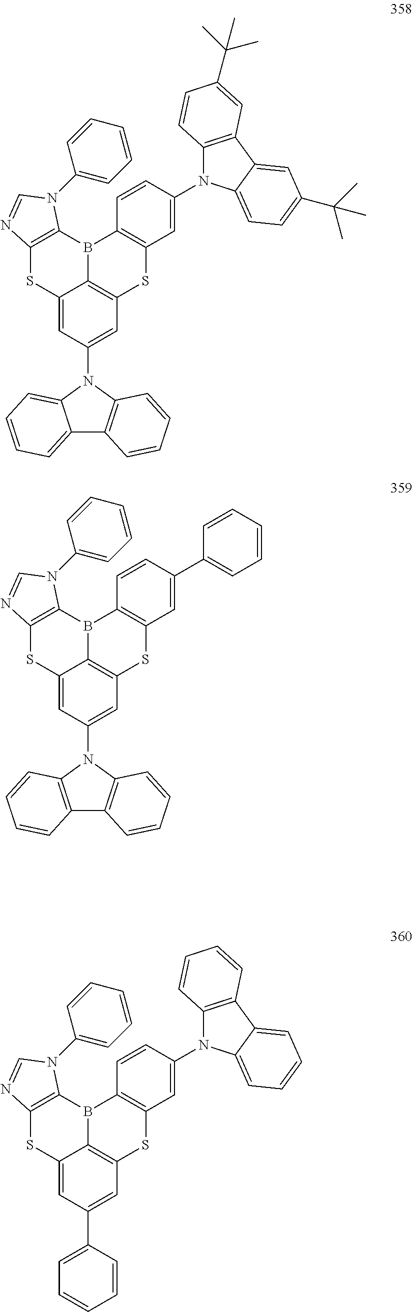 [SMILES: CC(C)(C)c1ccc2c(c1)c1cc(C(C)(C)C)ccc1n2-c1ccc2c(c1)Sc1cc(-n3c4ccccc4c4ccccc43)cc3c1B2c1c(ncn1-c1ccccc1)S3 ; c1ccc(-c2ccc3c(c2)Sc2cc(-n4c5ccccc5c5ccccc54)cc4c2B3c2c(ncn2-c2ccccc2)S4)cc1 ; c1ccc(-c2cc3c4c(c2)Sc2ncn(-c5ccccc5)c2B4c2ccc(-n4c5ccccc5c5ccccc54)cc2S3)cc1]

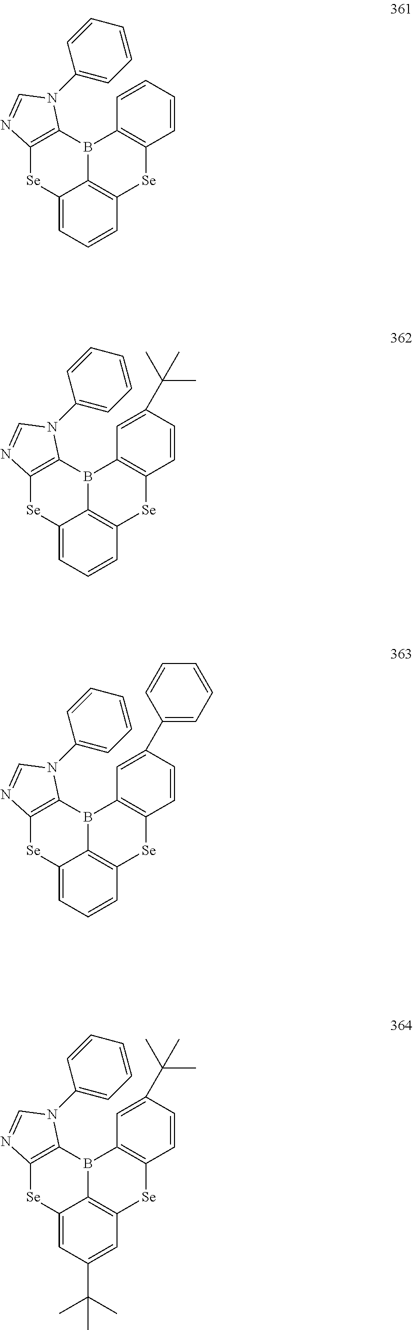 [SMILES: c1ccc(-n2cnc3c2B2c4ccccc4[Se]c4cccc(c42)[Se]3)cc1 ; CC(C)(C)c1ccc2c(c1)B1c3c(cccc3[Se]c3ncn(-c4ccccc4)c31)[Se]2 ; c1ccc(-c2ccc3c(c2)B2c4c(cccc4[Se]c4ncn(-c5ccccc5)c42)[Se]3)cc1 ; CC(C)(C)c1cc2c3c(c1)[Se]c1ncn(-c4ccccc4)c1B3c1cc(C(C)(C)C)ccc1[Se]2]

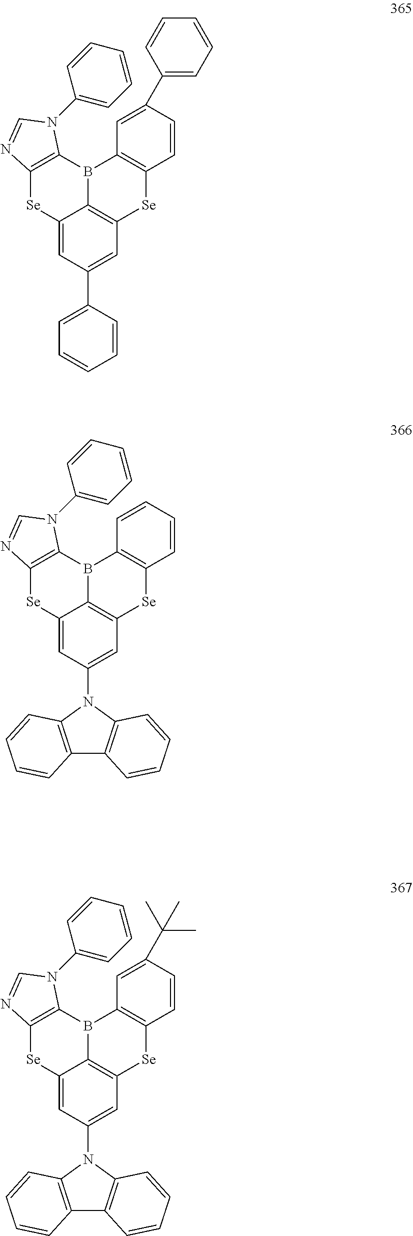 [SMILES: c1ccc(-c2cc3c4c(c2)[Se]c2ncn(-c5ccccc5)c2B4c2cc(-c4ccccc4)ccc2[Se]3)cc1 ; c1ccc(-n2cnc3c2B2c4ccccc4[Se]c4cc(-n5c6ccccc6c6ccccc65)cc(c42)[Se]3)cc1 ; CC(C)(C)c1ccc2c(c1)B1c3c(cc(-n4c5ccccc5c5ccccc54)cc3[Se]c3ncn(-c4ccccc4)c31)[Se]2]

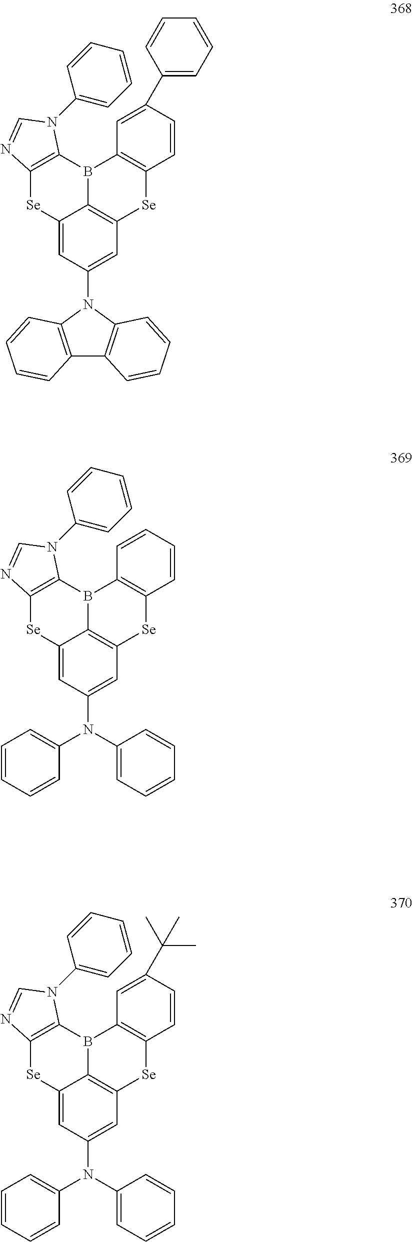 [SMILES: c1ccc(-c2ccc3c(c2)B2c4c(cc(-n5c6ccccc6c6ccccc65)cc4[Se]c4ncn(-c5ccccc5)c42)[Se]3)cc1 ; c1ccc(N(c2ccccc2)c2cc3c4c(c2)[Se]c2ncn(-c5ccccc5)c2B4c2ccccc2[Se]3)cc1 ; CC(C)(C)c1ccc2c(c1)B1c3c(cc(N(c4ccccc4)c4ccccc4)cc3[Se]c3ncn(-c4ccccc4)c31)[Se]2]

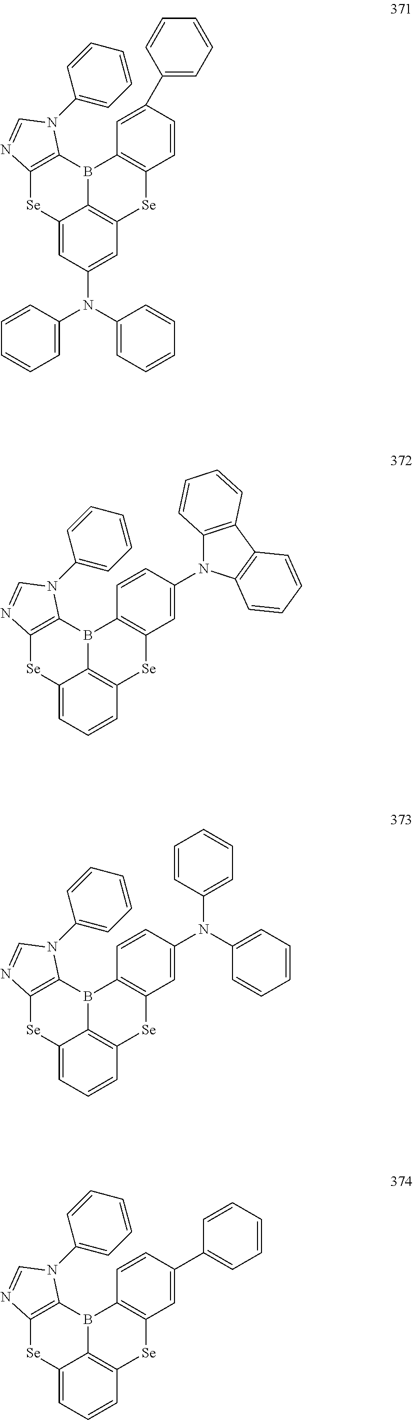 [SMILES: c1ccc(-c2ccc3c(c2)B2c4c(cc(N(c5ccccc5)c5ccccc5)cc4[Se]c4ncn(-c5ccccc5)c42)[Se]3)cc1 ; c1ccc(-n2cnc3c2B2c4ccc(-n5c6ccccc6c6ccccc65)cc4[Se]c4cccc(c42)[Se]3)cc1 ; c1ccc(N(c2ccccc2)c2ccc3c(c2)[Se]c2cccc4c2B3c2c(ncn2-c2ccccc2)[Se]4)cc1 ; c1ccc(-c2ccc3c(c2)[Se]c2cccc4c2B3c2c(ncn2-c2ccccc2)[Se]4)cc1]

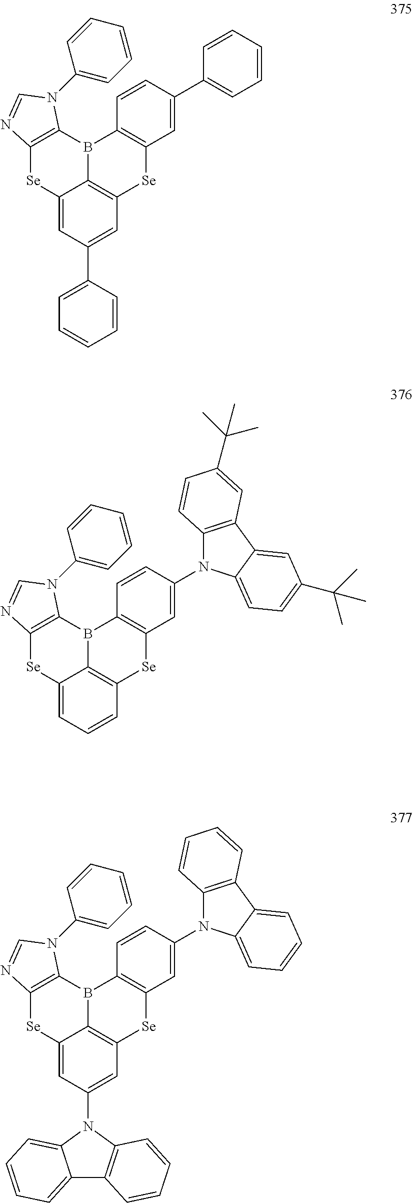 [SMILES: c1ccc(-c2ccc3c(c2)[Se]c2cc(-c4ccccc4)cc4c2B3c2c(ncn2-c2ccccc2)[Se]4)cc1 ; CC(C)(C)c1ccc2c(c1)c1cc(C(C)(C)C)ccc1n2-c1ccc2c(c1)[Se]c1cccc3c1B2c1c(ncn1-c1ccccc1)[Se]3 ; c1ccc(-n2cnc3c2B2c4ccc(-n5c6ccccc6c6ccccc65)cc4[Se]c4cc(-n5c6ccccc6c6ccccc65)cc(c42)[Se]3)cc1]

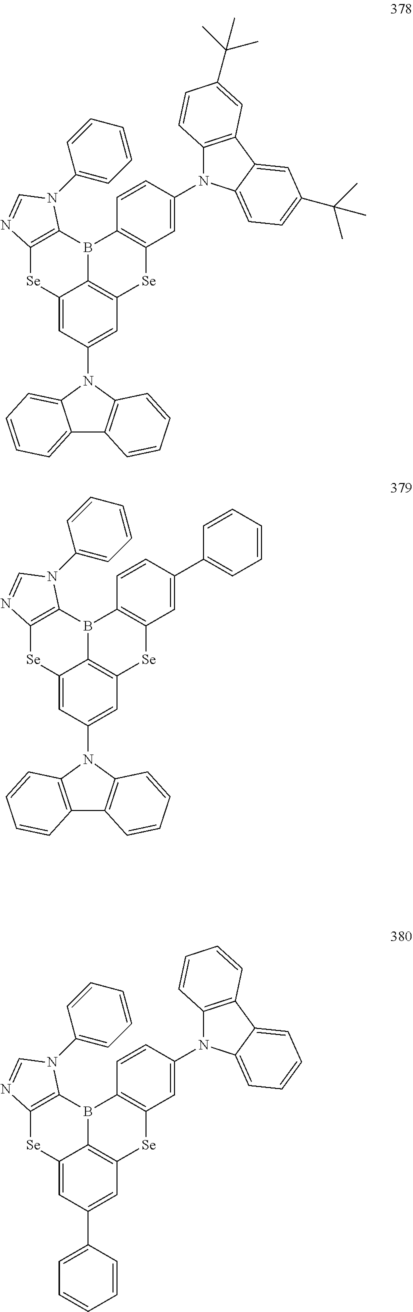 [SMILES: CC(C)(C)c1ccc2c(c1)c1cc(C(C)(C)C)ccc1n2-c1ccc2c(c1)[Se]c1cc(-n3c4ccccc4c4ccccc43)cc3c1B2c1c(ncn1-c1ccccc1)[Se]3 ; c1ccc(-c2ccc3c(c2)[Se]c2cc(-n4c5ccccc5c5ccccc54)cc4c2B3c2c(ncn2-c2ccccc2)[Se]4)cc1 ; c1ccc(-c2cc3c4c(c2)[Se]c2ncn(-c5ccccc5)c2B4c2ccc(-n4c5ccccc5c5ccccc54)cc2[Se]3)cc1]

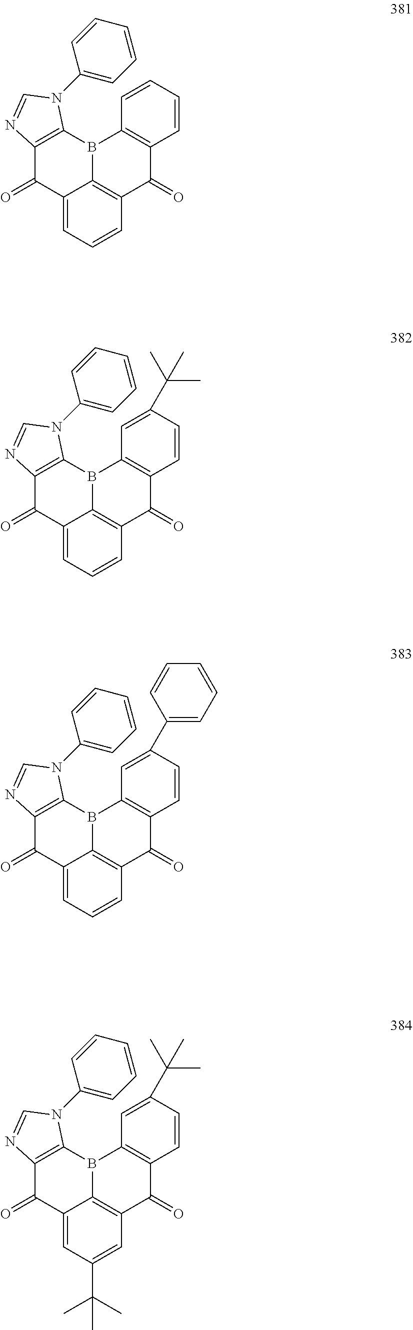 [SMILES: O=C1c2ccccc2B2c3c1cccc3C(=O)c1ncn(-c3ccccc3)c12 ; CC(C)(C)c1ccc2c(c1)B1c3c(cccc3C(=O)c3ncn(-c4ccccc4)c31)C2=O ; O=C1c2ccc(-c3ccccc3)cc2B2c3c1cccc3C(=O)c1ncn(-c3ccccc3)c12 ; CC(C)(C)c1ccc2c(c1)B1c3c(cc(C(C)(C)C)cc3C(=O)c3ncn(-c4ccccc4)c31)C2=O]

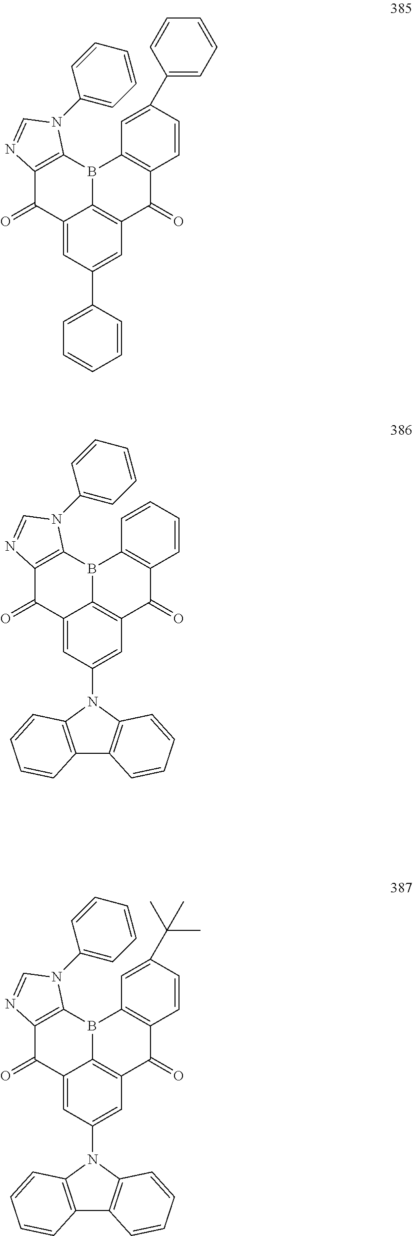 [SMILES: O=C1c2ccc(-c3ccccc3)cc2B2c3c1cc(-c1ccccc1)cc3C(=O)c1ncn(-c3ccccc3)c12 ; O=C1c2ccccc2B2c3c1cc(-n1c4ccccc4c4ccccc41)cc3C(=O)c1ncn(-c3ccccc3)c12 ; CC(C)(C)c1ccc2c(c1)B1c3c(cc(-n4c5ccccc5c5ccccc54)cc3C(=O)c3ncn(-c4ccccc4)c31)C2=O]

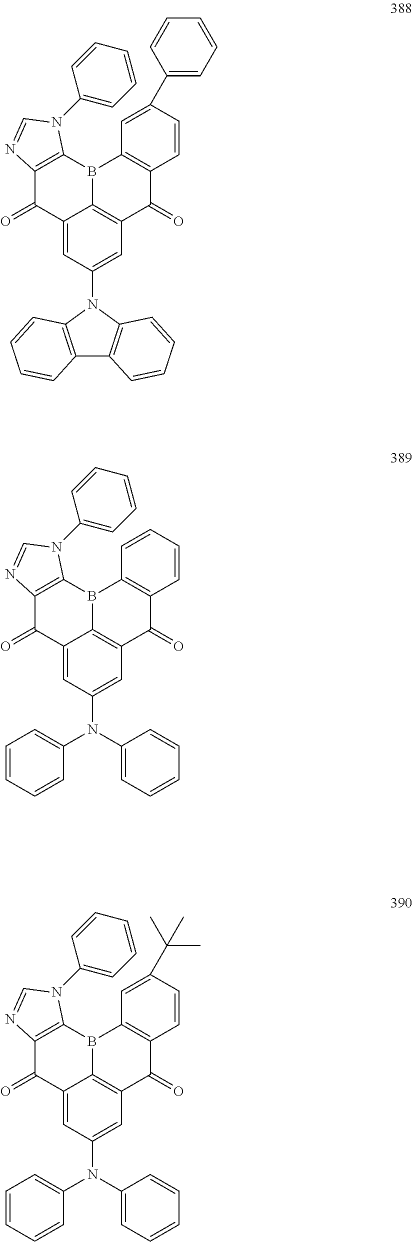 [SMILES: O=C1c2ccc(-c3ccccc3)cc2B2c3c1cc(-n1c4ccccc4c4ccccc41)cc3C(=O)c1ncn(-c3ccccc3)c12 ; O=C1c2ccccc2B2c3c1cc(N(c1ccccc1)c1ccccc1)cc3C(=O)c1ncn(-c3ccccc3)c12 ; CC(C)(C)c1ccc2c(c1)B1c3c(cc(N(c4ccccc4)c4ccccc4)cc3C(=O)c3ncn(-c4ccccc4)c31)C2=O]

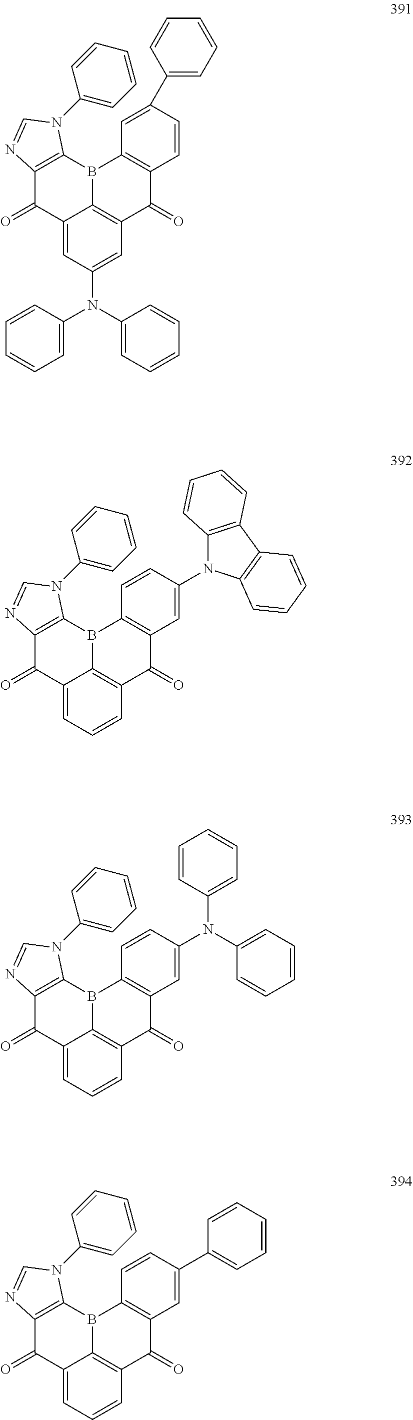 [SMILES: O=C1c2ccc(-c3ccccc3)cc2B2c3c1cc(N(c1ccccc1)c1ccccc1)cc3C(=O)c1ncn(-c3ccccc3)c12 ; O=C1c2cc(-n3c4ccccc4c4ccccc43)ccc2B2c3c1cccc3C(=O)c1ncn(-c3ccccc3)c12 ; O=C1c2cc(N(c3ccccc3)c3ccccc3)ccc2B2c3c1cccc3C(=O)c1ncn(-c3ccccc3)c12 ; O=C1c2cc(-c3ccccc3)ccc2B2c3c1cccc3C(=O)c1ncn(-c3ccccc3)c12]

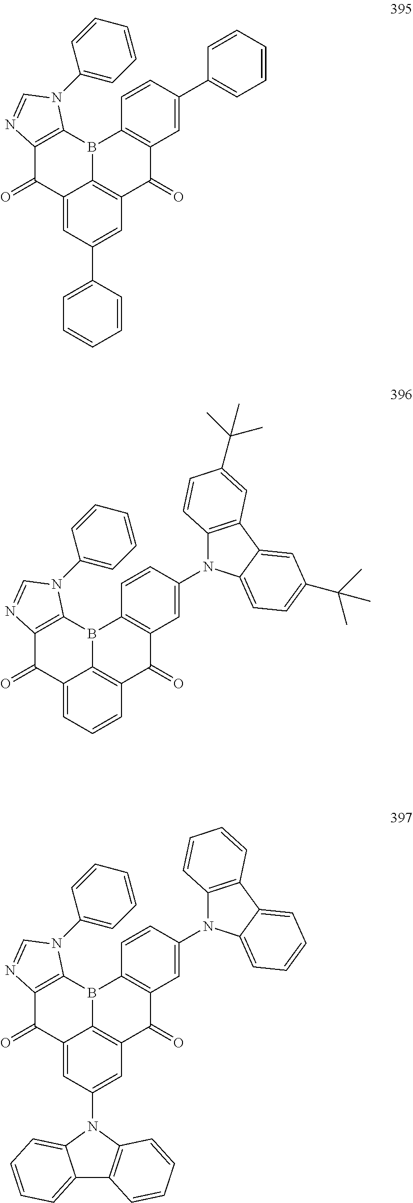 [SMILES: O=C1c2cc(-c3ccccc3)ccc2B2c3c1cc(-c1ccccc1)cc3C(=O)c1ncn(-c3ccccc3)c12 ; CC(C)(C)c1ccc2c(c1)c1cc(C(C)(C)C)ccc1n2-c1ccc2c(c1)C(=O)c1cccc3c1B2c1c(ncn1-c1ccccc1)C3=O ; O=C1c2cc(-n3c4ccccc4c4ccccc43)ccc2B2c3c1cc(-n1c4ccccc4c4ccccc41)cc3C(=O)c1ncn(-c3ccccc3)c12]

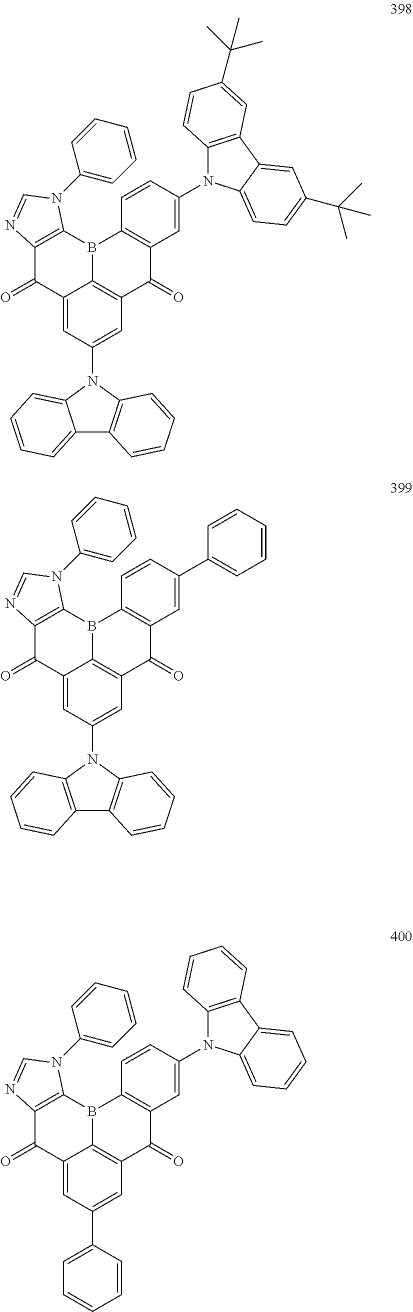 [SMILES: CC(C)(C)c1ccc2c(c1)c1cc(C(C)(C)C)ccc1n2-c1ccc2c(c1)C(=O)c1cc(-n3c4ccccc4c4ccccc43)cc3c1B2c1c(ncn1-c1ccccc1)C3=O ; O=C1c2cc(-c3ccccc3)ccc2B2c3c1cc(-n1c4ccccc4c4ccccc41)cc3C(=O)c1ncn(-c3ccccc3)c12 ; O=C1c2cc(-n3c4ccccc4c4ccccc43)ccc2B2c3c1cc(-c1ccccc1)cc3C(=O)c1ncn(-c3ccccc3)c12]

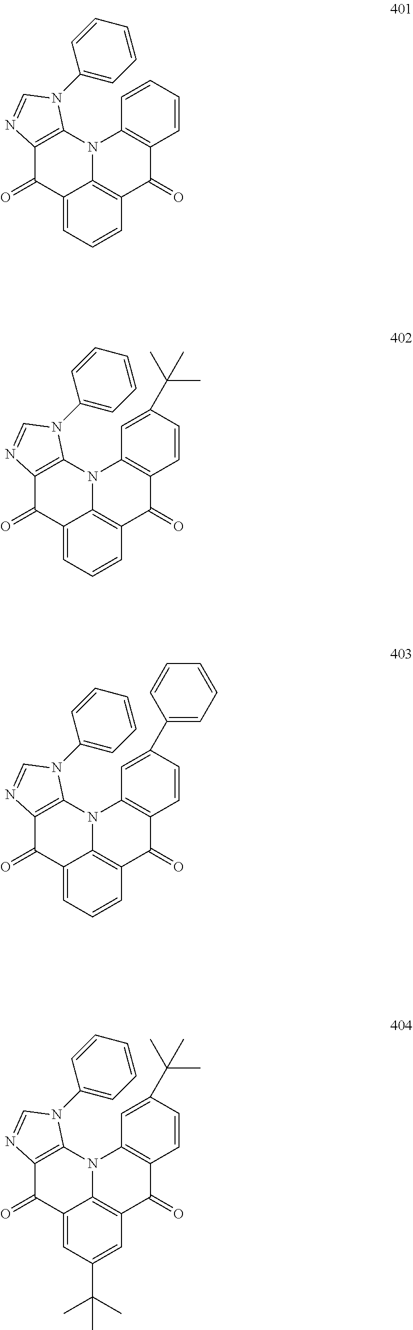 [SMILES: O=c1c2ccccc2n2c3c1cccc3c(=O)c1ncn(-c3ccccc3)c12 ; CC(C)(C)c1ccc2c(=O)c3cccc4c(=O)c5ncn(-c6ccccc6)c5n(c2c1)c34 ; O=c1c2ccc(-c3ccccc3)cc2n2c3c1cccc3c(=O)c1ncn(-c3ccccc3)c12 ; CC(C)(C)c1cc2c(=O)c3ccc(C(C)(C)C)cc3n3c2c(c1)c(=O)c1ncn(-c2ccccc2)c13]

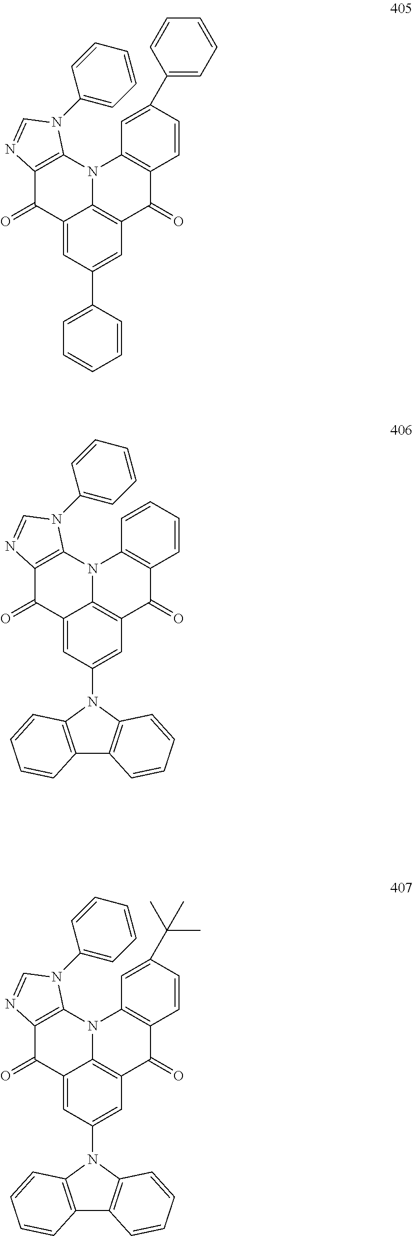 [SMILES: O=c1c2ccc(-c3ccccc3)cc2n2c3c1cc(-c1ccccc1)cc3c(=O)c1ncn(-c3ccccc3)c12 ; O=c1c2ccccc2n2c3c1cc(-n1c4ccccc4c4ccccc41)cc3c(=O)c1ncn(-c3ccccc3)c12 ; CC(C)(C)c1ccc2c(=O)c3cc(-n4c5ccccc5c5ccccc54)cc4c(=O)c5ncn(-c6ccccc6)c5n(c2c1)c34]

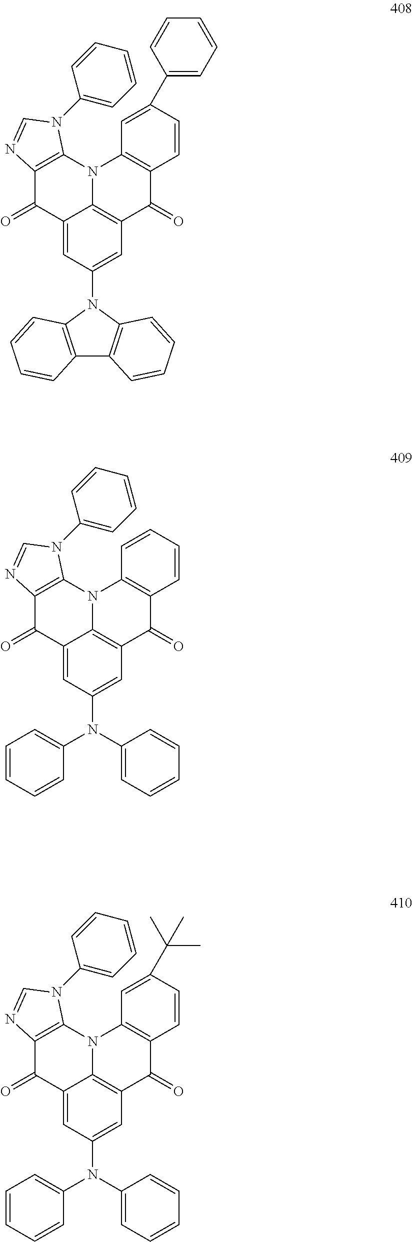 [SMILES: O=c1c2ccc(-c3ccccc3)cc2n2c3c1cc(-n1c4ccccc4c4ccccc41)cc3c(=O)c1ncn(-c3ccccc3)c12 ; O=c1c2ccccc2n2c3c1cc(N(c1ccccc1)c1ccccc1)cc3c(=O)c1ncn(-c3ccccc3)c12 ; CC(C)(C)c1ccc2c(=O)c3cc(N(c4ccccc4)c4ccccc4)cc4c(=O)c5ncn(-c6ccccc6)c5n(c2c1)c34]

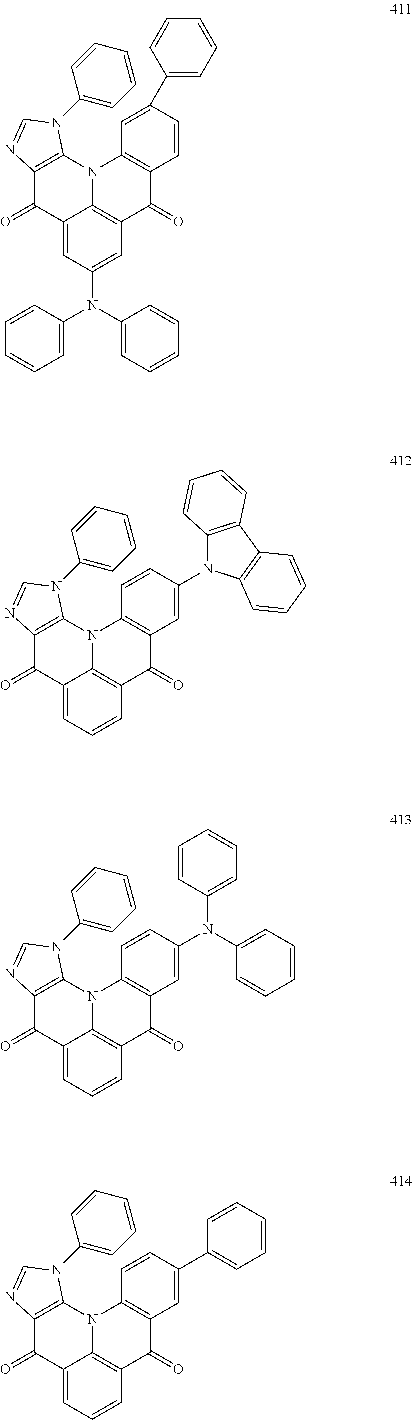 [SMILES: O=c1c2ccc(-c3ccccc3)cc2n2c3c1cc(N(c1ccccc1)c1ccccc1)cc3c(=O)c1ncn(-c3ccccc3)c12 ; O=c1c2cc(-n3c4ccccc4c4ccccc43)ccc2n2c3c1cccc3c(=O)c1ncn(-c3ccccc3)c12 ; O=c1c2cc(N(c3ccccc3)c3ccccc3)ccc2n2c3c1cccc3c(=O)c1ncn(-c3ccccc3)c12 ; O=c1c2cc(-c3ccccc3)ccc2n2c3c1cccc3c(=O)c1ncn(-c3ccccc3)c12]

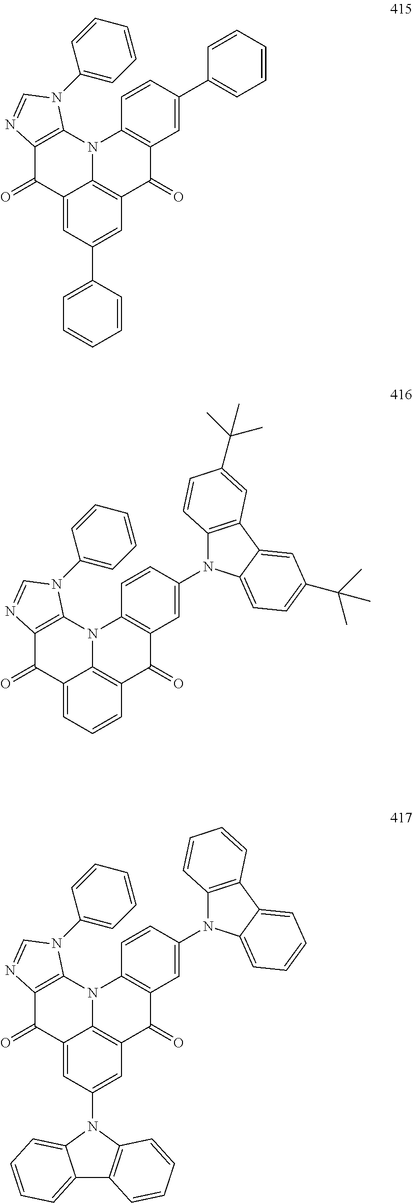 [SMILES: O=c1c2cc(-c3ccccc3)ccc2n2c3c1cc(-c1ccccc1)cc3c(=O)c1ncn(-c3ccccc3)c12 ; CC(C)(C)c1ccc2c(c1)c1cc(C(C)(C)C)ccc1n2-c1ccc2c(c1)c(=O)c1cccc3c(=O)c4ncn(-c5ccccc5)c4n2c13 ; O=c1c2cc(-n3c4ccccc4c4ccccc43)ccc2n2c3c1cc(-n1c4ccccc4c4ccccc41)cc3c(=O)c1ncn(-c3ccccc3)c12]

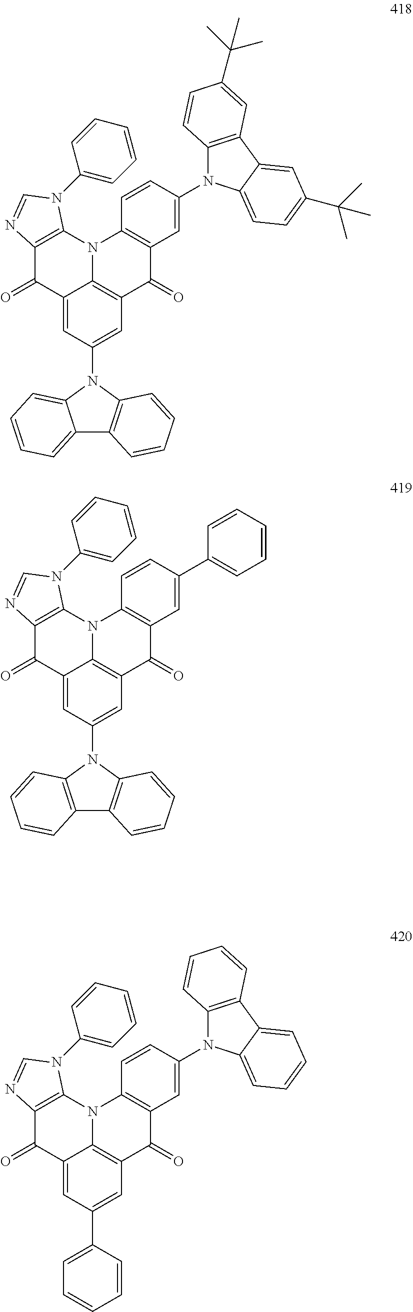 [SMILES: CC(C)(C)c1ccc2c(c1)c1cc(C(C)(C)C)ccc1n2-c1ccc2c(c1)c(=O)c1cc(-n3c4ccccc4c4ccccc43)cc3c(=O)c4ncn(-c5ccccc5)c4n2c13 ; O=c1c2cc(-c3ccccc3)ccc2n2c3c1cc(-n1c4ccccc4c4ccccc41)cc3c(=O)c1ncn(-c3ccccc3)c12 ; O=c1c2cc(-n3c4ccccc4c4ccccc43)ccc2n2c3c1cc(-c1ccccc1)cc3c(=O)c1ncn(-c3ccccc3)c12]

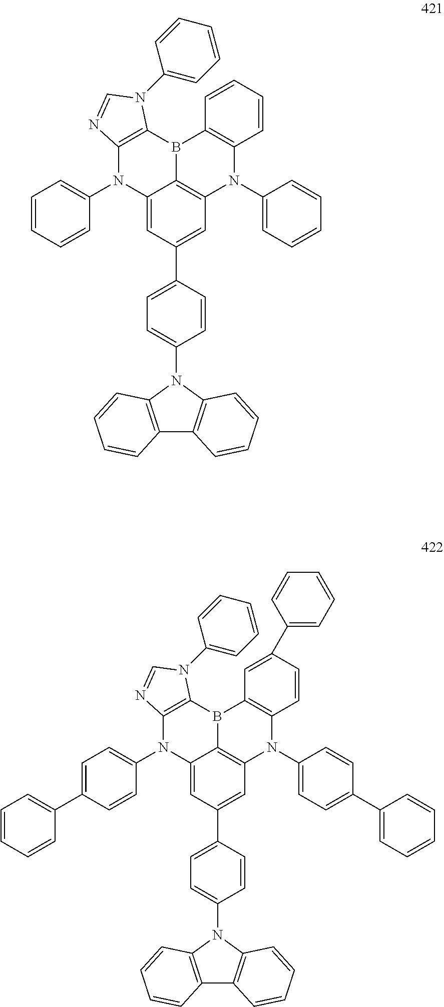 [SMILES: c1ccc(N2c3ccccc3B3c4c2cc(-c2ccc(-n5c6ccccc6c6ccccc65)cc2)cc4N(c2ccccc2)c2ncn(-c4ccccc4)c23)cc1 ; c1ccc(-c2ccc(N3c4ccc(-c5ccccc5)cc4B4c5c3cc(-c3ccc(-n6c7ccccc7c7ccccc76)cc3)cc5N(c3ccc(-c5ccccc5)cc3)c3ncn(-c5ccccc5)c34)cc2)cc1]

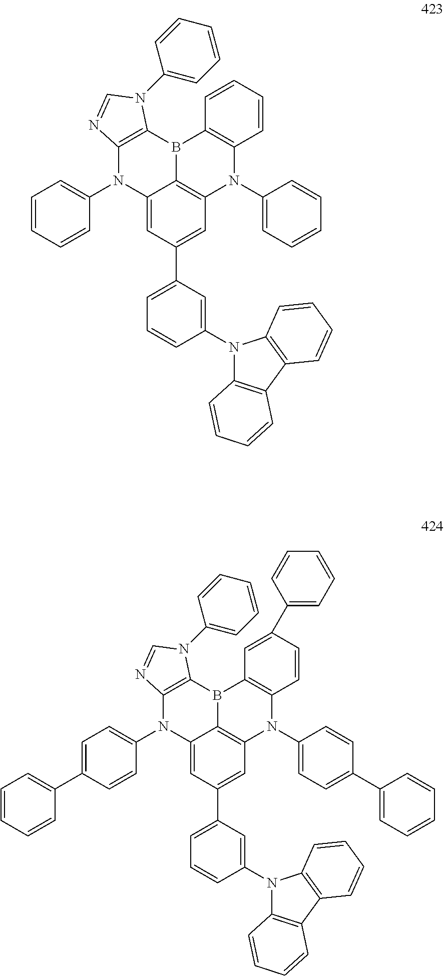 [SMILES: c1ccc(N2c3ccccc3B3c4c2cc(-c2cccc(-n5c6ccccc6c6ccccc65)c2)cc4N(c2ccccc2)c2ncn(-c4ccccc4)c23)cc1 ; c1ccc(-c2ccc(N3c4ccc(-c5ccccc5)cc4B4c5c3cc(-c3cccc(-n6c7ccccc7c7ccccc76)c3)cc5N(c3ccc(-c5ccccc5)cc3)c3ncn(-c5ccccc5)c34)cc2)cc1]

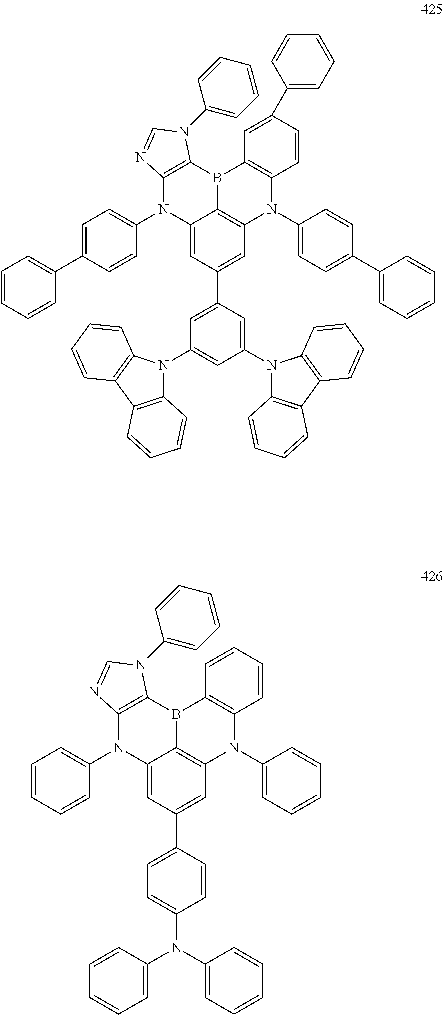 [SMILES: c1ccc(-c2ccc(N3c4ccc(-c5ccccc5)cc4B4c5c3cc(-c3cc(-n6c7ccccc7c7ccccc76)cc(-n6c7ccccc7c7ccccc76)c3)cc5N(c3ccc(-c5ccccc5)cc3)c3ncn(-c5ccccc5)c34)cc2)cc1 ; c1ccc(N(c2ccccc2)c2ccc(-c3cc4c5c(c3)N(c3ccccc3)c3ncn(-c6ccccc6)c3B5c3ccccc3N4c3ccccc3)cc2)cc1]

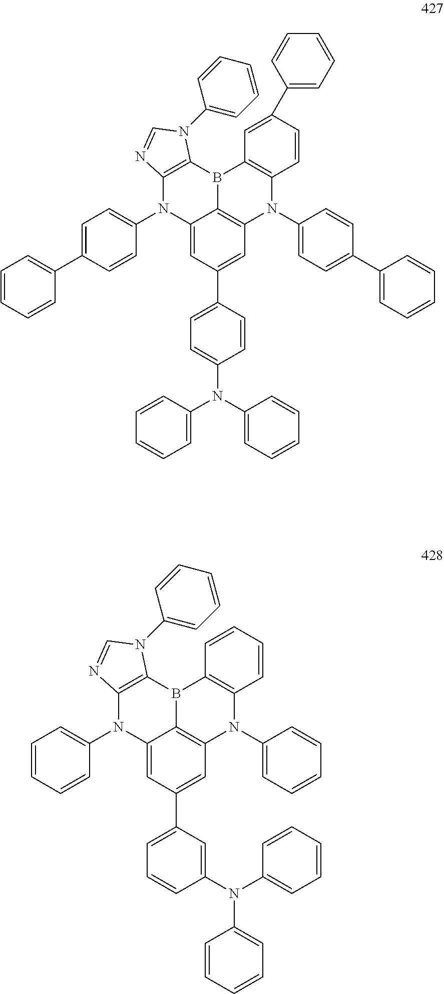 [SMILES: c1ccc(-c2ccc(N3c4ccc(-c5ccccc5)cc4B4c5c3cc(-c3ccc(N(c6ccccc6)c6ccccc6)cc3)cc5N(c3ccc(-c5ccccc5)cc3)c3ncn(-c5ccccc5)c34)cc2)cc1 ; c1ccc(N(c2ccccc2)c2cccc(-c3cc4c5c(c3)N(c3ccccc3)c3ncn(-c6ccccc6)c3B5c3ccccc3N4c3ccccc3)c2)cc1]

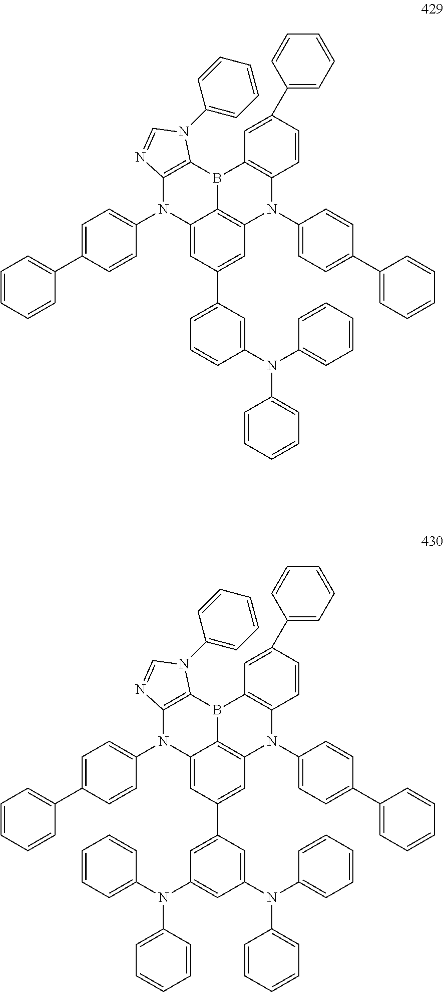 [SMILES: c1ccc(-c2ccc(N3c4ccc(-c5ccccc5)cc4B4c5c3cc(-c3cccc(N(c6ccccc6)c6ccccc6)c3)cc5N(c3ccc(-c5ccccc5)cc3)c3ncn(-c5ccccc5)c34)cc2)cc1 ; c1ccc(-c2ccc(N3c4ccc(-c5ccccc5)cc4B4c5c3cc(-c3cc(N(c6ccccc6)c6ccccc6)cc(N(c6ccccc6)c6ccccc6)c3)cc5N(c3ccc(-c5ccccc5)cc3)c3ncn(-c5ccccc5)c34)cc2)cc1]

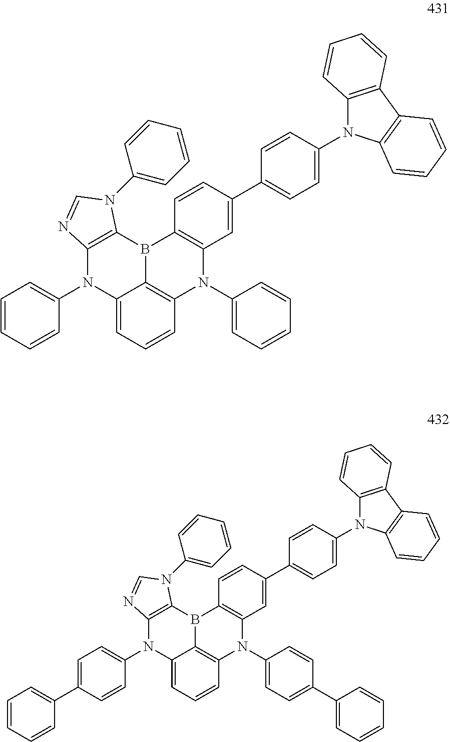 [SMILES: c1ccc(N2c3cc(-c4ccc(-n5c6ccccc6c6ccccc65)cc4)ccc3B3c4c2cccc4N(c2ccccc2)c2ncn(-c4ccccc4)c23)cc1 ; c1ccc(-c2ccc(N3c4cc(-c5ccc(-n6c7ccccc7c7ccccc76)cc5)ccc4B4c5c3cccc5N(c3ccc(-c5ccccc5)cc3)c3ncn(-c5ccccc5)c34)cc2)cc1]

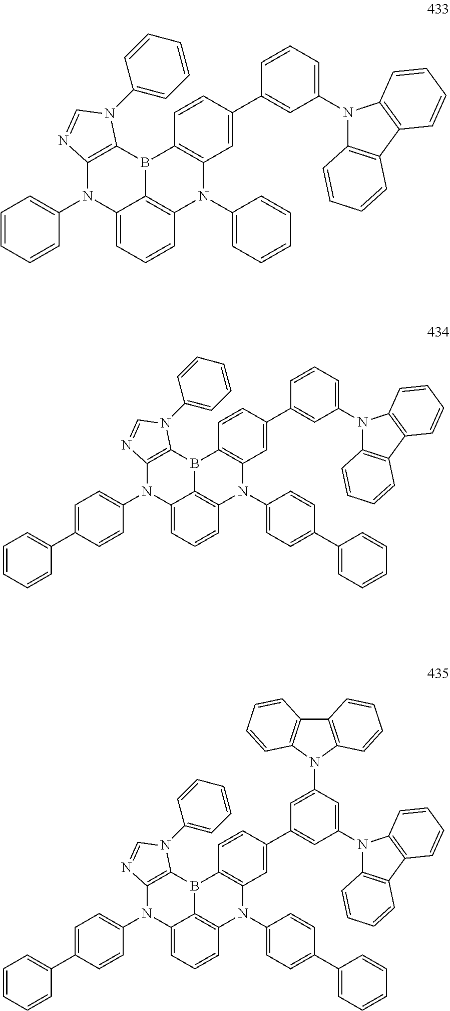 [SMILES: c1ccc(N2c3cc(-c4cccc(-n5c6ccccc6c6ccccc65)c4)ccc3B3c4c2cccc4N(c2ccccc2)c2ncn(-c4ccccc4)c23)cc1 ; c1ccc(-c2ccc(N3c4cc(-c5cccc(-n6c7ccccc7c7ccccc76)c5)ccc4B4c5c3cccc5N(c3ccc(-c5ccccc5)cc3)c3ncn(-c5ccccc5)c34)cc2)cc1 ; c1ccc(-c2ccc(N3c4cc(-c5cc(-n6c7ccccc7c7ccccc76)cc(-n6c7ccccc7c7ccccc76)c5)ccc4B4c5c3cccc5N(c3ccc(-c5ccccc5)cc3)c3ncn(-c5ccccc5)c34)cc2)cc1]

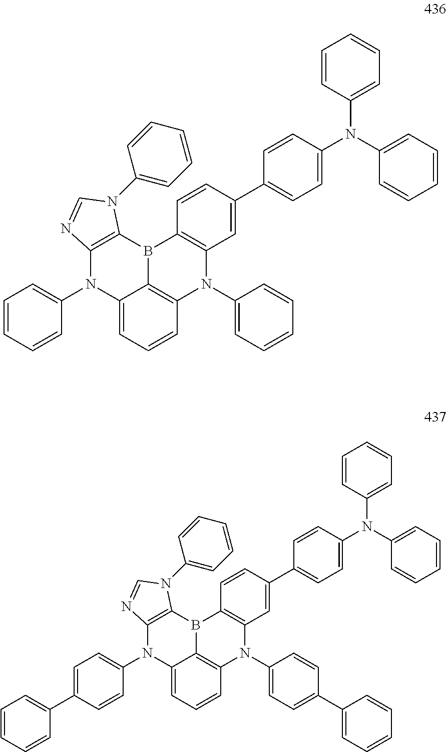 [SMILES: c1ccc(N(c2ccccc2)c2ccc(-c3ccc4c(c3)N(c3ccccc3)c3cccc5c3B4c3c(ncn3-c3ccccc3)N5c3ccccc3)cc2)cc1 ; c1ccc(-c2ccc(N3c4cc(-c5ccc(N(c6ccccc6)c6ccccc6)cc5)ccc4B4c5c3cccc5N(c3ccc(-c5ccccc5)cc3)c3ncn(-c5ccccc5)c34)cc2)cc1]

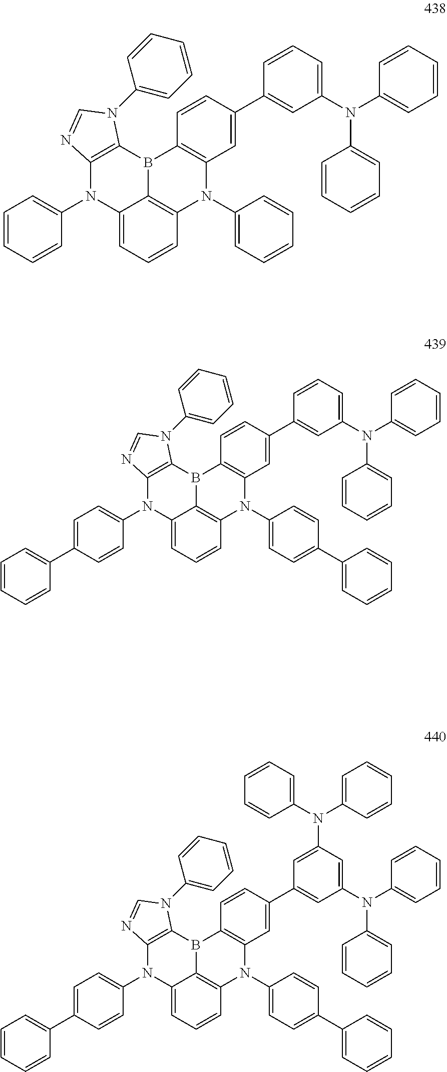 [SMILES: c1ccc(N(c2ccccc2)c2cccc(-c3ccc4c(c3)N(c3ccccc3)c3cccc5c3B4c3c(ncn3-c3ccccc3)N5c3ccccc3)c2)cc1 ; c1ccc(-c2ccc(N3c4cc(-c5cccc(N(c6ccccc6)c6ccccc6)c5)ccc4B4c5c3cccc5N(c3ccc(-c5ccccc5)cc3)c3ncn(-c5ccccc5)c34)cc2)cc1 ; c1ccc(-c2ccc(N3c4cc(-c5cc(N(c6ccccc6)c6ccccc6)cc(N(c6ccccc6)c6ccccc6)c5)ccc4B4c5c3cccc5N(c3ccc(-c5ccccc5)cc3)c3ncn(-c5ccccc5)c34)cc2)cc1]

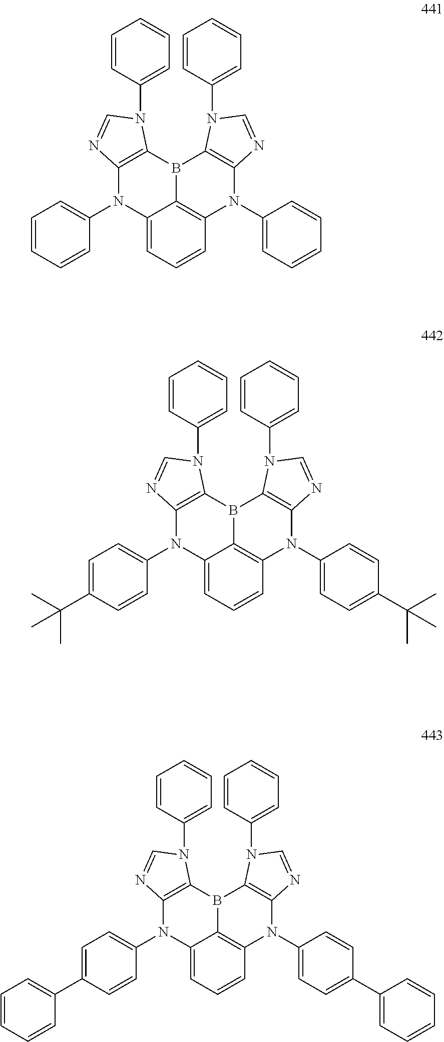 [SMILES: c1ccc(N2c3cccc4c3B(c3c2ncn3-c2ccccc2)c2c(ncn2-c2ccccc2)N4c2ccccc2)cc1 ; CC(C)(C)c1ccc(N2c3cccc4c3B(c3c2ncn3-c2ccccc2)c2c(ncn2-c2ccccc2)N4c2ccc(C(C)(C)C)cc2)cc1 ; c1ccc(-c2ccc(N3c4cccc5c4B(c4c3ncn4-c3ccccc3)c3c(ncn3-c3ccccc3)N5c3ccc(-c4ccccc4)cc3)cc2)cc1]

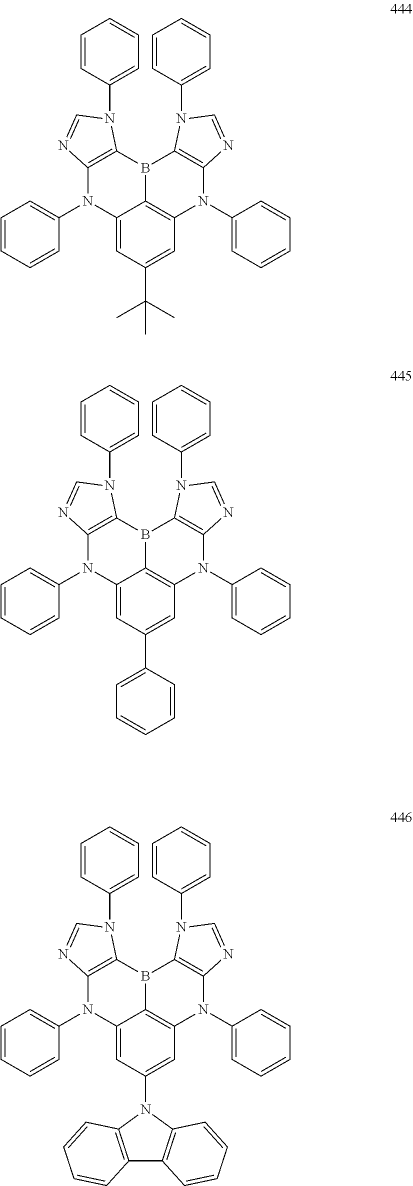 [SMILES: CC(C)(C)c1cc2c3c(c1)N(c1ccccc1)c1ncn(-c4ccccc4)c1B3c1c(ncn1-c1ccccc1)N2c1ccccc1 ; c1ccc(-c2cc3c4c(c2)N(c2ccccc2)c2ncn(-c5ccccc5)c2B4c2c(ncn2-c2ccccc2)N3c2ccccc2)cc1 ; c1ccc(N2c3cc(-n4c5ccccc5c5ccccc54)cc4c3B(c3c2ncn3-c2ccccc2)c2c(ncn2-c2ccccc2)N4c2ccccc2)cc1]

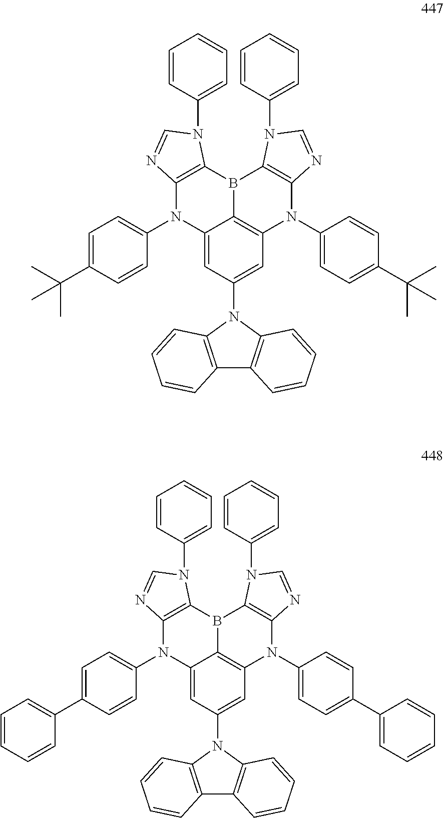 [SMILES: CC(C)(C)c1ccc(N2c3cc(-n4c5ccccc5c5ccccc54)cc4c3B(c3c2ncn3-c2ccccc2)c2c(ncn2-c2ccccc2)N4c2ccc(C(C)(C)C)cc2)cc1 ; c1ccc(-c2ccc(N3c4cc(-n5c6ccccc6c6ccccc65)cc5c4B(c4c3ncn4-c3ccccc3)c3c(ncn3-c3ccccc3)N5c3ccc(-c4ccccc4)cc3)cc2)cc1]

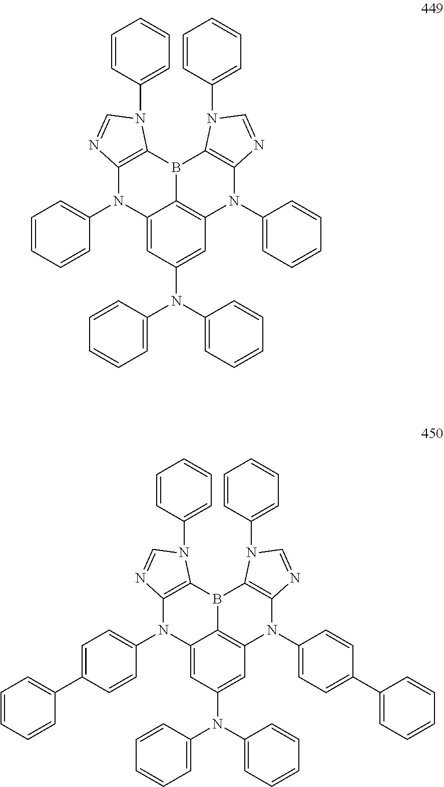 [SMILES: c1ccc(N(c2ccccc2)c2cc3c4c(c2)N(c2ccccc2)c2ncn(-c5ccccc5)c2B4c2c(ncn2-c2ccccc2)N3c2ccccc2)cc1 ; c1ccc(-c2ccc(N3c4cc(N(c5ccccc5)c5ccccc5)cc5c4B(c4c3ncn4-c3ccccc3)c3c(ncn3-c3ccccc3)N5c3ccc(-c4ccccc4)cc3)cc2)cc1]

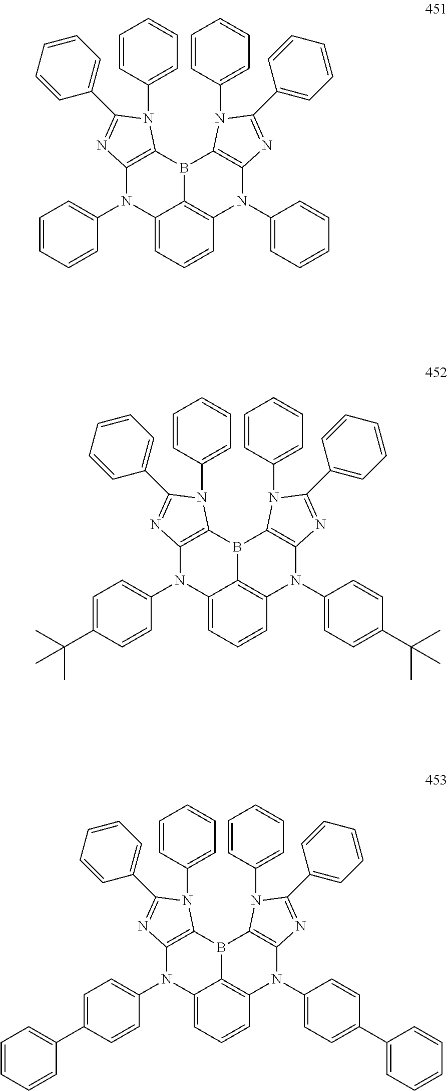 [SMILES: c1ccc(-c2nc3c(n2-c2ccccc2)B2c4c(cccc4N(c4ccccc4)c4nc(-c5ccccc5)n(-c5ccccc5)c42)N3c2ccccc2)cc1 ; CC(C)(C)c1ccc(N2c3cccc4c3B(c3c2nc(-c2ccccc2)n3-c2ccccc2)c2c(nc(-c3ccccc3)n2-c2ccccc2)N4c2ccc(C(C)(C)C)cc2)cc1 ; c1ccc(-c2ccc(N3c4cccc5c4B(c4c3nc(-c3ccccc3)n4-c3ccccc3)c3c(nc(-c4ccccc4)n3-c3ccccc3)N5c3ccc(-c4ccccc4)cc3)cc2)cc1]

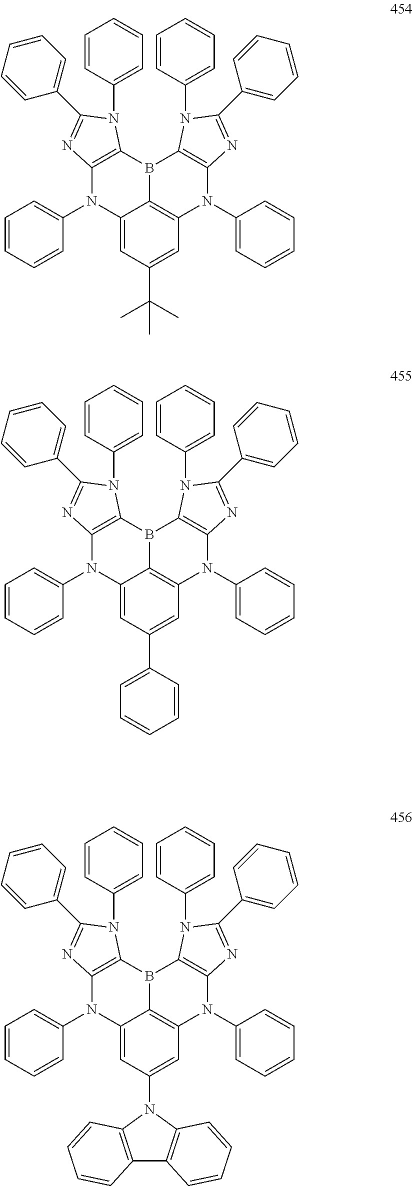 [SMILES: CC(C)(C)c1cc2c3c(c1)N(c1ccccc1)c1nc(-c4ccccc4)n(-c4ccccc4)c1B3c1c(nc(-c3ccccc3)n1-c1ccccc1)N2c1ccccc1 ; c1ccc(-c2cc3c4c(c2)N(c2ccccc2)c2nc(-c5ccccc5)n(-c5ccccc5)c2B4c2c(nc(-c4ccccc4)n2-c2ccccc2)N3c2ccccc2)cc1 ; c1ccc(-c2nc3c(n2-c2ccccc2)B2c4c(cc(-n5c6ccccc6c6ccccc65)cc4N(c4ccccc4)c4nc(-c5ccccc5)n(-c5ccccc5)c42)N3c2ccccc2)cc1]

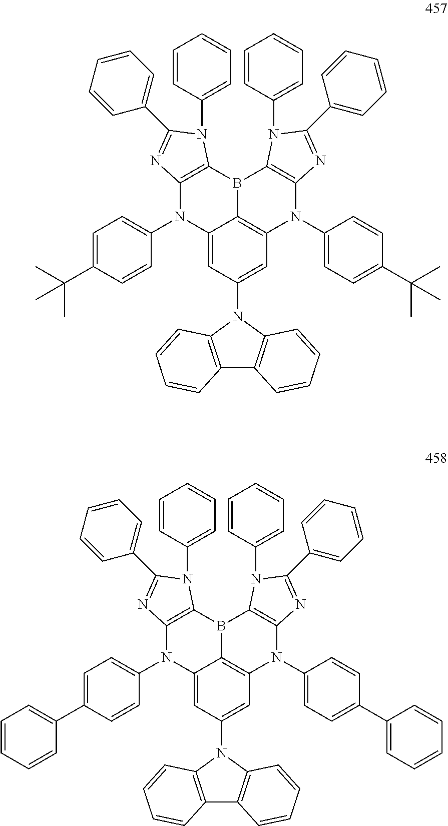 [SMILES: CC(C)(C)c1ccc(N2c3cc(-n4c5ccccc5c5ccccc54)cc4c3B(c3c2nc(-c2ccccc2)n3-c2ccccc2)c2c(nc(-c3ccccc3)n2-c2ccccc2)N4c2ccc(C(C)(C)C)cc2)cc1 ; c1ccc(-c2ccc(N3c4cc(-n5c6ccccc6c6ccccc65)cc5c4B(c4c3nc(-c3ccccc3)n4-c3ccccc3)c3c(nc(-c4ccccc4)n3-c3ccccc3)N5c3ccc(-c4ccccc4)cc3)cc2)cc1]

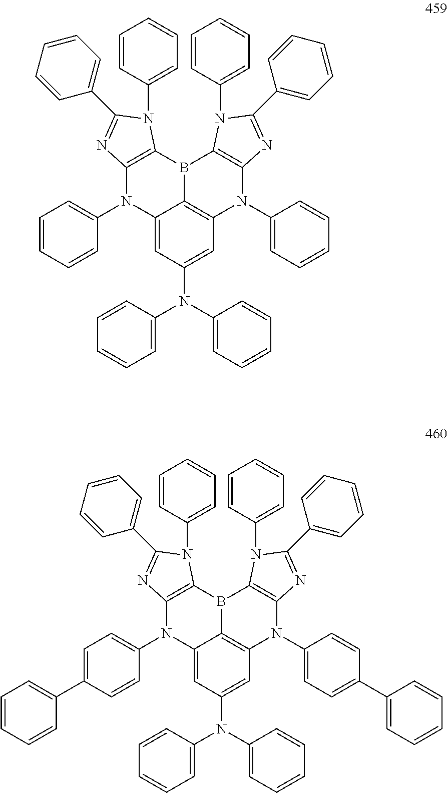 [SMILES: c1ccc(-c2nc3c(n2-c2ccccc2)B2c4c(cc(N(c5ccccc5)c5ccccc5)cc4N(c4ccccc4)c4nc(-c5ccccc5)n(-c5ccccc5)c42)N3c2ccccc2)cc1 ; c1ccc(-c2ccc(N3c4cc(N(c5ccccc5)c5ccccc5)cc5c4B(c4c3nc(-c3ccccc3)n4-c3ccccc3)c3c(nc(-c4ccccc4)n3-c3ccccc3)N5c3ccc(-c4ccccc4)cc3)cc2)cc1]

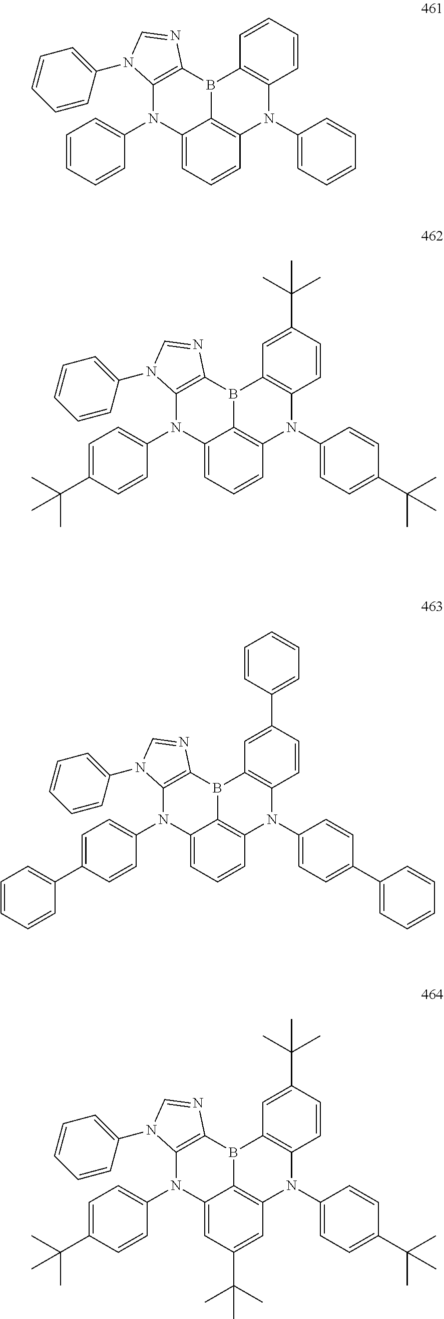

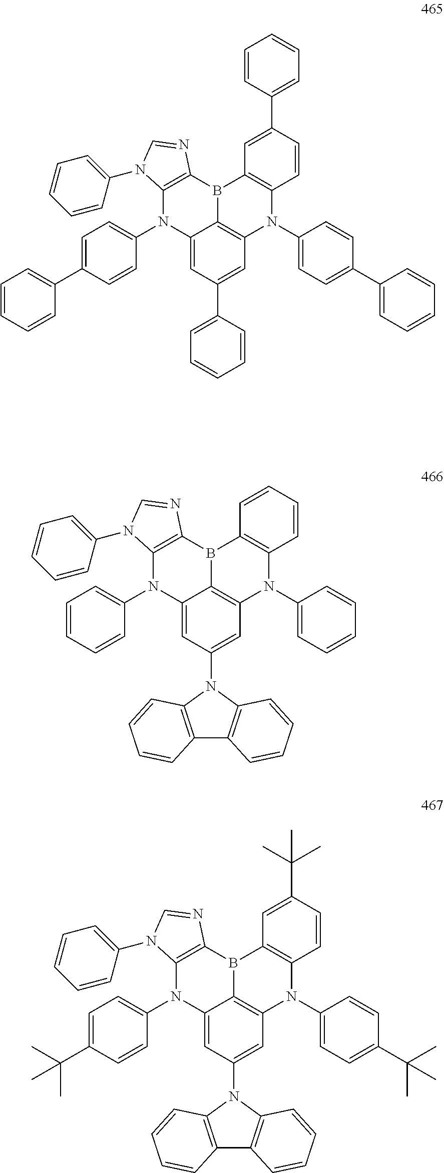 [SMILES: c1ccc(-c2ccc(N3c4ccc(-c5ccccc5)cc4B4c5ncn(-c6ccccc6)c5N(c5ccc(-c6ccccc6)cc5)c5cc(-c6ccccc6)cc3c54)cc2)cc1 ; c1ccc(N2c3ccccc3B3c4ncn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3B3c4ncn(-c5ccccc5)c4N(c4ccc(C(C)(C)C)cc4)c4cc(-n5c6ccccc6c6ccccc65)cc2c43)cc1]

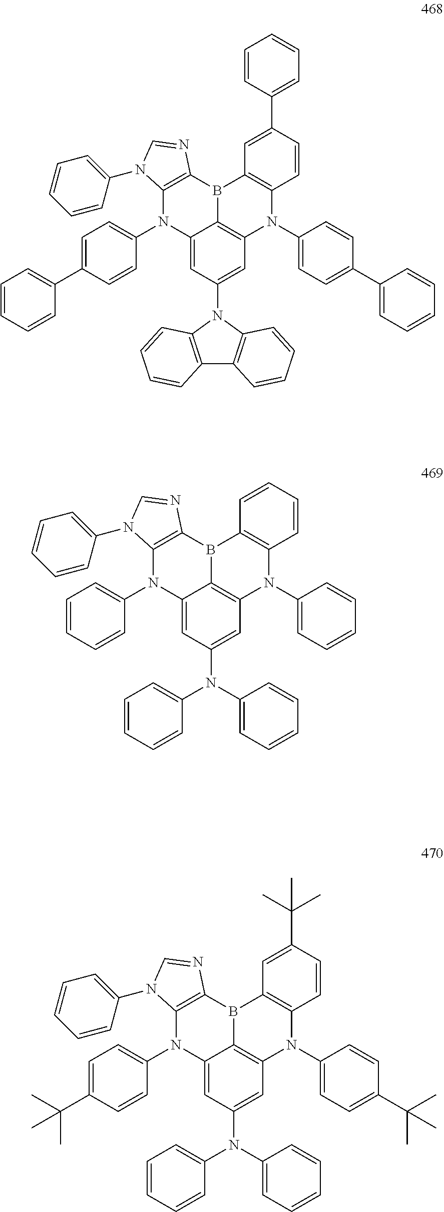 [SMILES: c1ccc(-c2ccc(N3c4ccc(-c5ccccc5)cc4B4c5ncn(-c6ccccc6)c5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2c(ncn2-c2ccccc2)B4c2ccccc2N3c2ccccc2)cc1 ; CC(C)(C)c1ccc(N2c3ccc(C(C)(C)C)cc3B3c4ncn(-c5ccccc5)c4N(c4ccc(C(C)(C)C)cc4)c4cc(N(c5ccccc5)c5ccccc5)cc2c43)cc1]

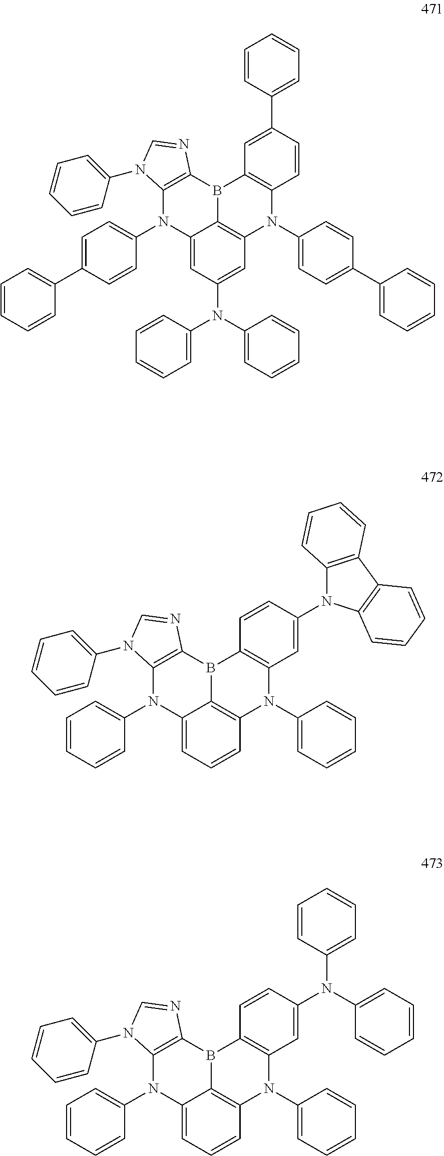 [SMILES: c1ccc(-c2ccc(N3c4ccc(-c5ccccc5)cc4B4c5ncn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4ncn(-c5ccccc5)c4N(c4ccccc4)c4cccc2c43)cc1 ; c1ccc(N(c2ccccc2)c2ccc3c(c2)N(c2ccccc2)c2cccc4c2B3c2ncn(-c3ccccc3)c2N4c2ccccc2)cc1]

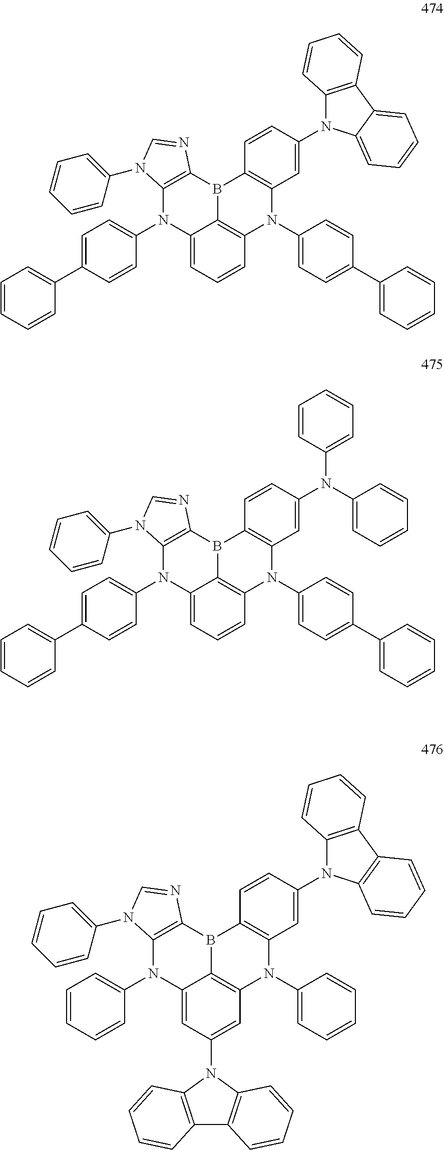 [SMILES: c1ccc(-c2ccc(N3c4cc(-n5c6ccccc6c6ccccc65)ccc4B4c5ncn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5ncn(-c6ccccc6)c5N(c5ccc(-c6ccccc6)cc5)c5cccc3c54)cc2)cc1 ; c1ccc(N2c3cc(-n4c5ccccc5c5ccccc54)ccc3B3c4ncn(-c5ccccc5)c4N(c4ccccc4)c4cc(-n5c6ccccc6c6ccccc65)cc2c43)cc1]

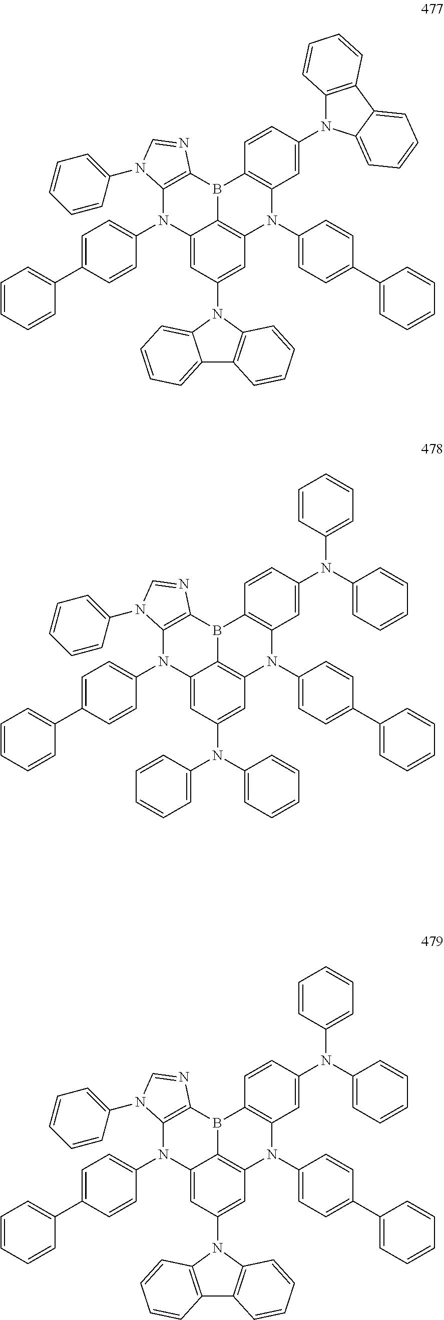 [SMILES: c1ccc(-c2ccc(N3c4cc(-n5c6ccccc6c6ccccc65)ccc4B4c5ncn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cc(N(c5ccccc5)c5ccccc5)ccc4B4c5ncn(-c6ccccc6)c5N(c5ccc(-c6ccccc6)cc5)c5cc(N(c6ccccc6)c6ccccc6)cc3c54)cc2)cc1 ; c1ccc(-c2ccc(N3c4cc(N(c5ccccc5)c5ccccc5)ccc4B4c5ncn(-c6ccccc6)c5N(c5ccc(-c6ccccc6)cc5)c5cc(-n6c7ccccc7c7ccccc76)cc3c54)cc2)cc1]

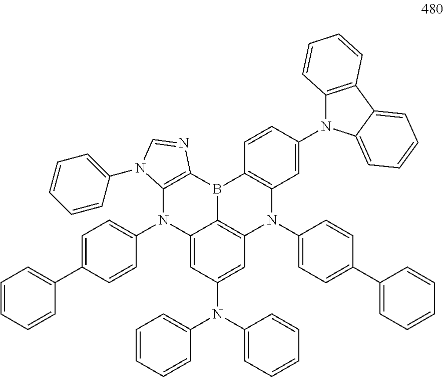 [SMILES: c1ccc(-c2ccc(N3c4cc(-n5c6ccccc6c6ccccc65)ccc4B4c5ncn(-c6ccccc6)c5N(c5ccc(-c6ccccc6)cc5)c5cc(N(c6ccccc6)c6ccccc6)cc3c54)cc2)cc1]

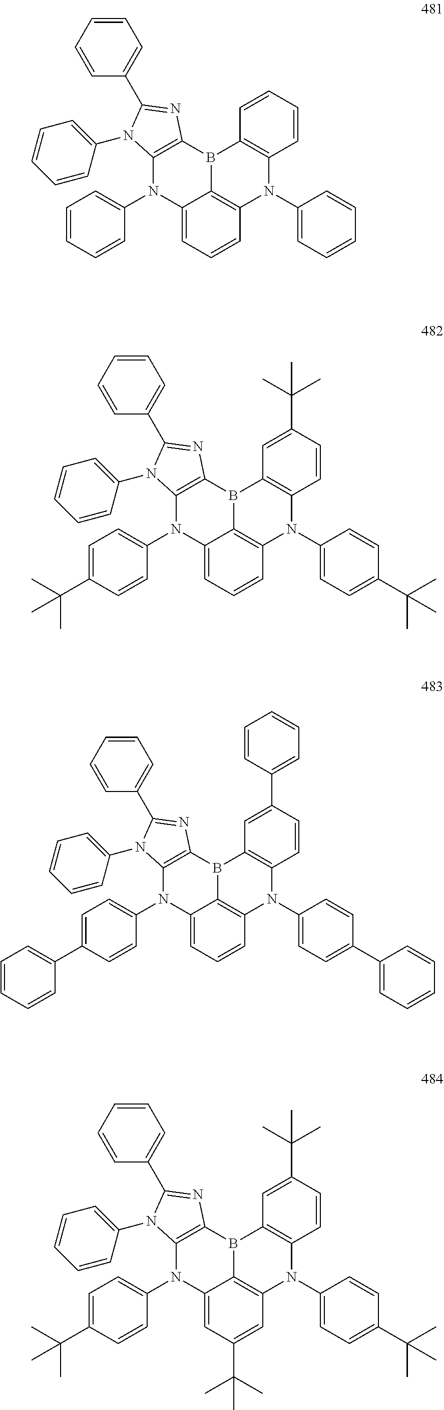

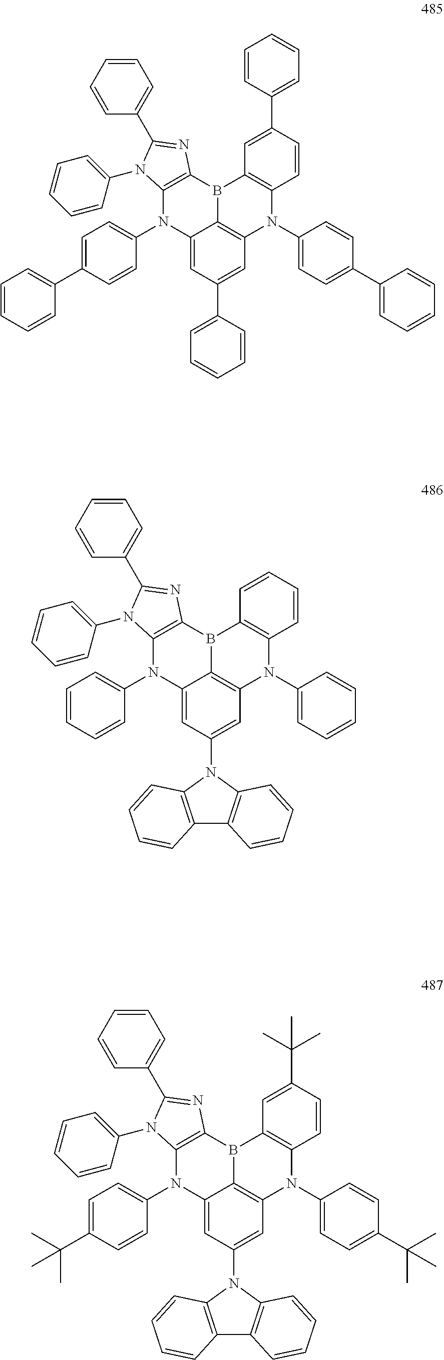 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(-c6ccccc6)cc3c54)cc2)cc1 ; c1ccc(-c2nc3c(n2-c2ccccc2)N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4N(c4ccc(C(C)(C)C)cc4)c4cc(-n5c6ccccc6c6ccccc65)cc2c43)cc1]

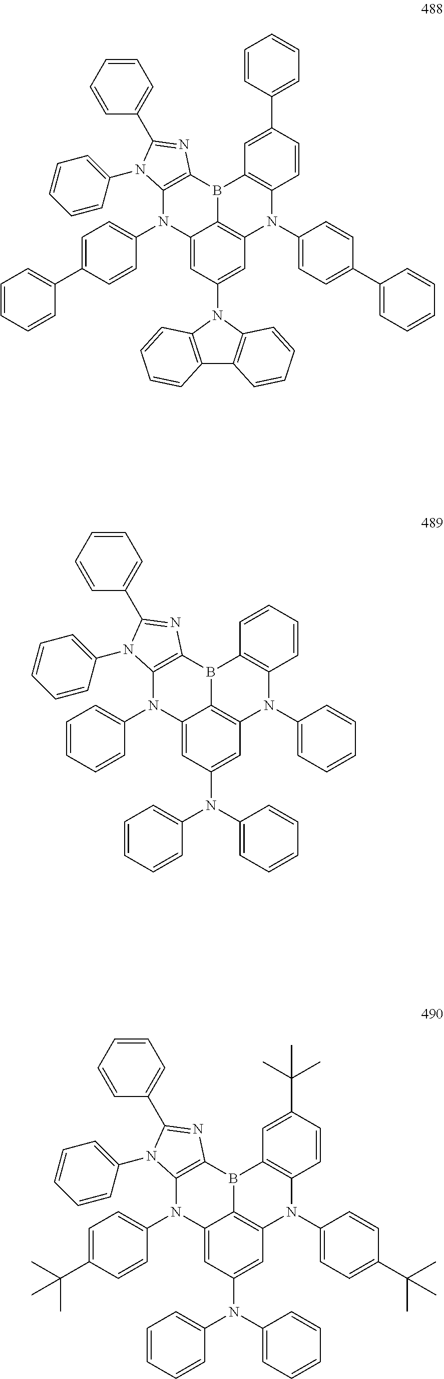 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7ccccc76)cc3c54)cc2)cc1 ; c1ccc(-c2nc3c(n2-c2ccccc2)N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4N(c4ccc(C(C)(C)C)cc4)c4cc(N(c5ccccc5)c5ccccc5)cc2c43)cc1]

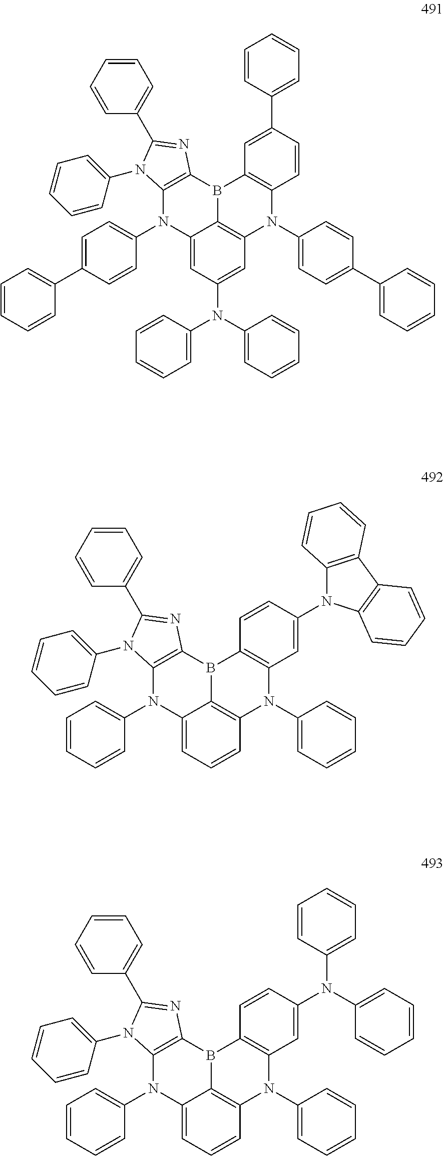 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(N(c6ccccc6)c6ccccc6)cc3c54)cc2)cc1 ; c1ccc(-c2nc3c(n2-c2ccccc2)N(c2ccccc2)c2cccc4c2B3c2ccc(-n3c5ccccc5c5ccccc53)cc2N4c2ccccc2)cc1 ; c1ccc(-c2nc3c(n2-c2ccccc2)N(c2ccccc2)c2cccc4c2B3c2ccc(N(c3ccccc3)c3ccccc3)cc2N4c2ccccc2)cc1]

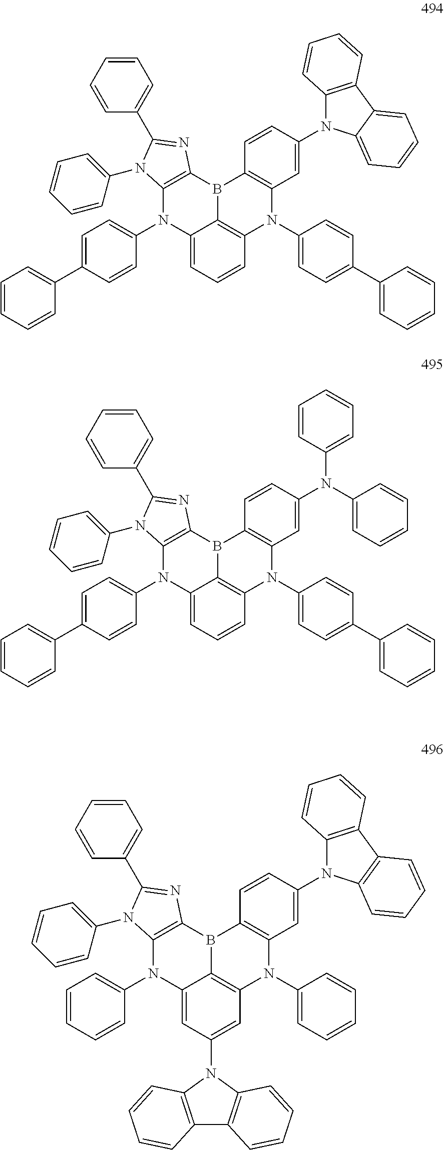 [SMILES: c1ccc(-c2ccc(N3c4cc(-n5c6ccccc6c6ccccc65)ccc4B4c5nc(-c6ccccc6)n(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cccc3c54)cc2)cc1 ; c1ccc(-c2nc3c(n2-c2ccccc2)N(c2ccccc2)c2cc(-n4c5ccccc5c5ccccc54)cc4c2B3c2ccc(-n3c5ccccc5c5ccccc53)cc2N4c2ccccc2)cc1]

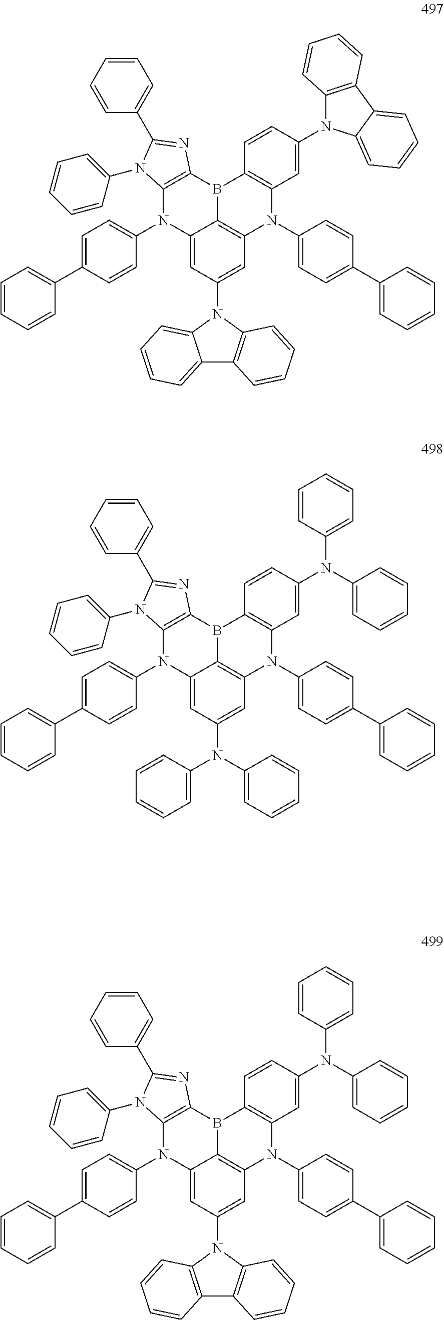 [SMILES: c1ccc(-c2ccc(N3c4cc(-n5c6ccccc6c6ccccc65)ccc4B4c5nc(-c6ccccc6)n(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cc(N(c6ccccc6)c6ccccc6)cc3c54)cc2)cc1 ; c1ccc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cc(-n6c7ccccc7c7ccccc76)cc3c54)cc2)cc1]

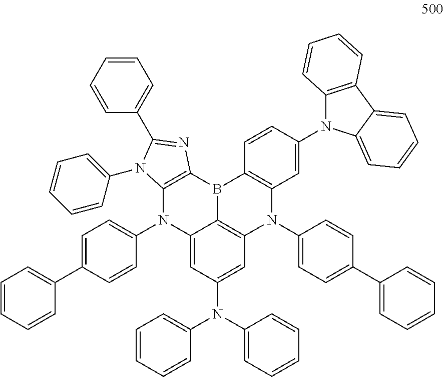 [SMILES: c1ccc(-c2ccc(N3c4cc(-n5c6ccccc6c6ccccc65)ccc4B4c5nc(-c6ccccc6)n(-c6ccccc6)c5N(c5ccc(-c6ccccc6)cc5)c5cc(N(c6ccccc6)c6ccccc6)cc3c54)cc2)cc1]

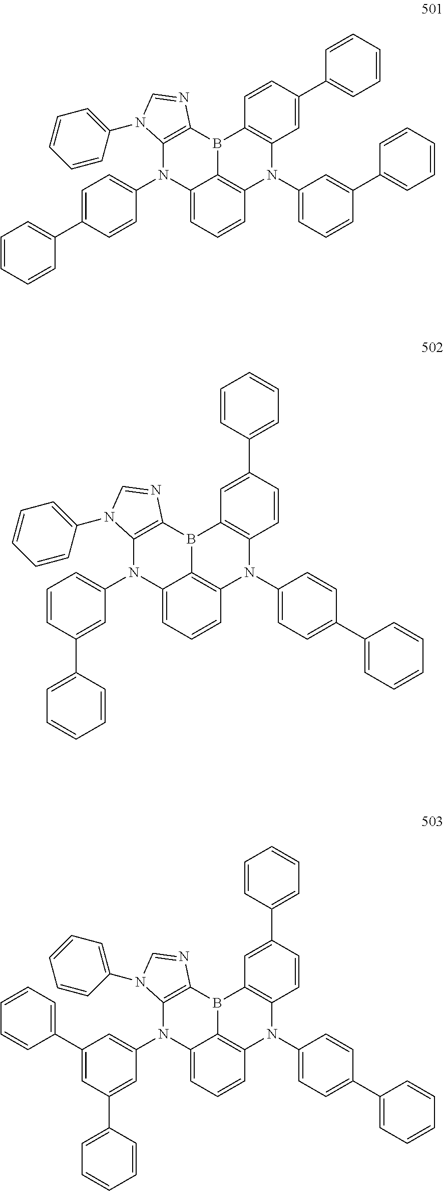 [SMILES: c1ccc(-c2ccc(N3c4cccc5c4B(c4ccc(-c6ccccc6)cc4N5c4cccc(-c5ccccc5)c4)c4ncn(-c5ccccc5)c43)cc2)cc1 ; c1ccc(-c2ccc(N3c4ccc(-c5ccccc5)cc4B4c5ncn(-c6ccccc6)c5N(c5cccc(-c6ccccc6)c5)c5cccc3c54)cc2)cc1 ; c1ccc(-c2ccc(N3c4ccc(-c5ccccc5)cc4B4c5ncn(-c6ccccc6)c5N(c5cc(-c6ccccc6)cc(-c6ccccc6)c5)c5cccc3c54)cc2)cc1]

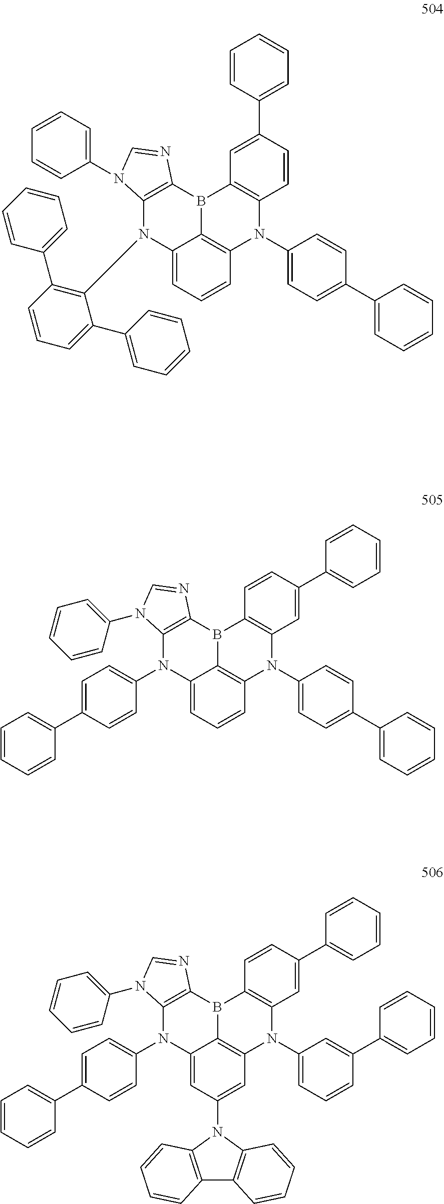 [SMILES: c1ccc(-c2ccc(N3c4ccc(-c5ccccc5)cc4B4c5ncn(-c6ccccc6)c5N(c5c(-c6ccccc6)cccc5-c5ccccc5)c5cccc3c54)cc2)cc1 ; c1ccc(-c2ccc(N3c4cc(-c5ccccc5)ccc4B4c5ncn(-c6ccccc6)c5N(c5ccc(-c6ccccc6)cc5)c5cccc3c54)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4ncn(-c5ccccc5)c43)cc2)cc1]

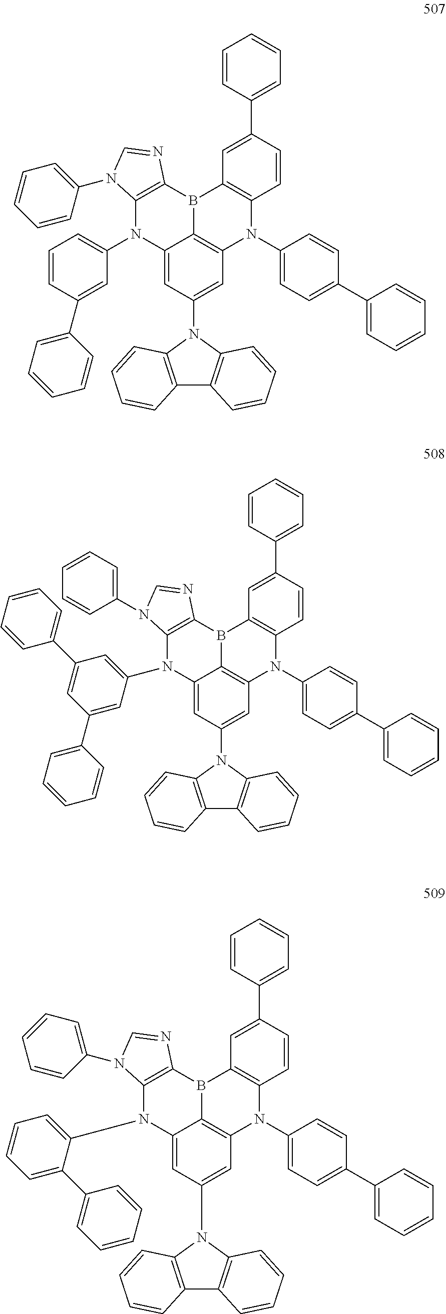 [SMILES: c1ccc(-c2ccc(N3c4ccc(-c5ccccc5)cc4B4c5ncn(-c6ccccc6)c5N(c5cccc(-c6ccccc6)c5)c5cc(-n6c7ccccc7c7ccccc76)cc3c54)cc2)cc1 ; c1ccc(-c2ccc(N3c4ccc(-c5ccccc5)cc4B4c5ncn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4ccc(-c5ccccc5)cc4B4c5ncn(-c6ccccc6)c5N(c5ccccc5-c5ccccc5)c5cc(-n6c7ccccc7c7ccccc76)cc3c54)cc2)cc1]

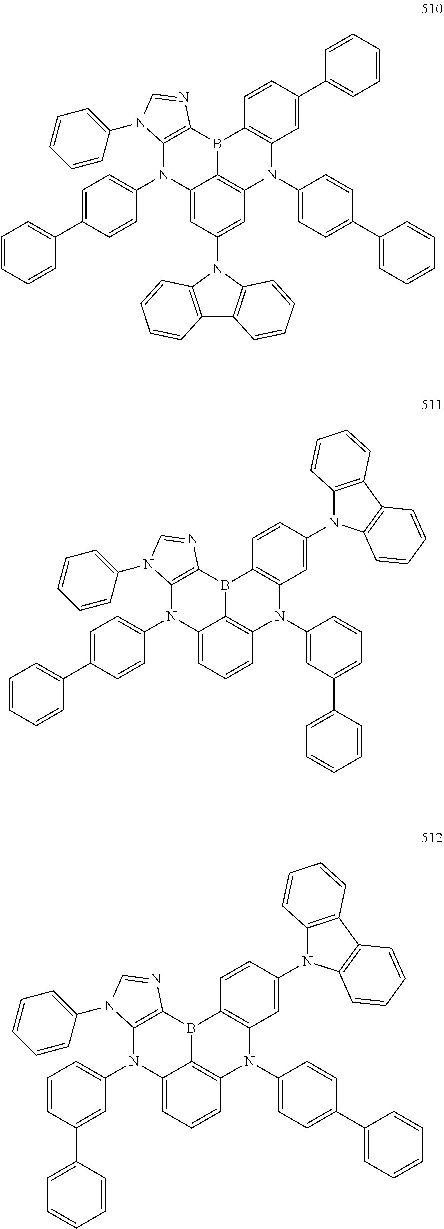 [SMILES: c1ccc(-c2ccc(N3c4cc(-c5ccccc5)ccc4B4c5ncn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cccc5c4B(c4ccc(-n6c7ccccc7c7ccccc76)cc4N5c4cccc(-c5ccccc5)c4)c4ncn(-c5ccccc5)c43)cc2)cc1 ; c1ccc(-c2ccc(N3c4cc(-n5c6ccccc6c6ccccc65)ccc4B4c5ncn(-c6ccccc6)c5N(c5cccc(-c6ccccc6)c5)c5cccc3c54)cc2)cc1]

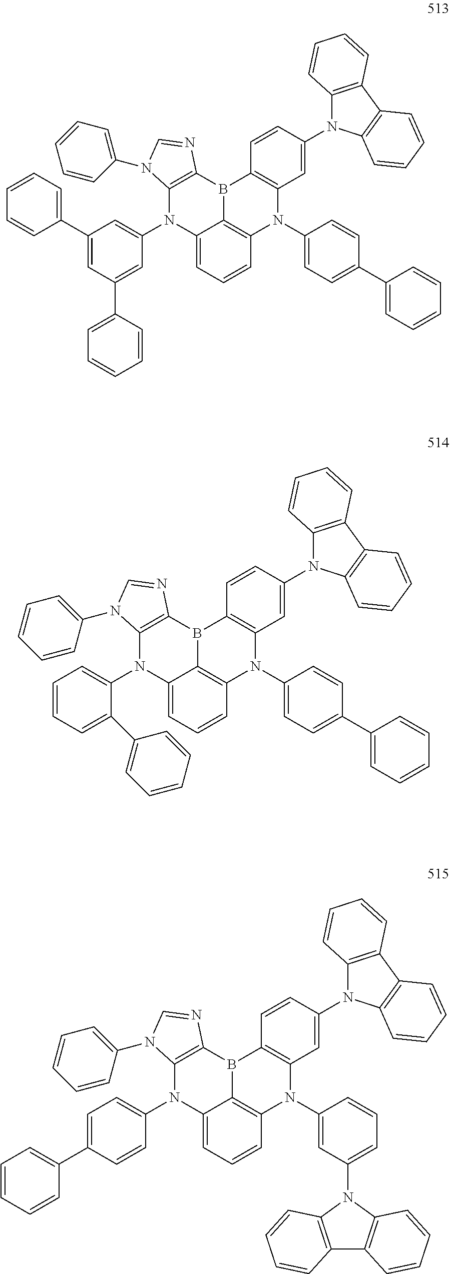 [SMILES: c1ccc(-c2ccc(N3c4cc(-n5c6ccccc6c6ccccc65)ccc4B4c5ncn(-c6ccccc6)c5N(c5cc(-c6ccccc6)cc(-c6ccccc6)c5)c5cccc3c54)cc2)cc1 ; c1ccc(-c2ccc(N3c4cc(-n5c6ccccc6c6ccccc65)ccc4B4c5ncn(-c6ccccc6)c5N(c5ccccc5-c5ccccc5)c5cccc3c54)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4ncn(-c5ccccc5)c43)cc2)cc1]

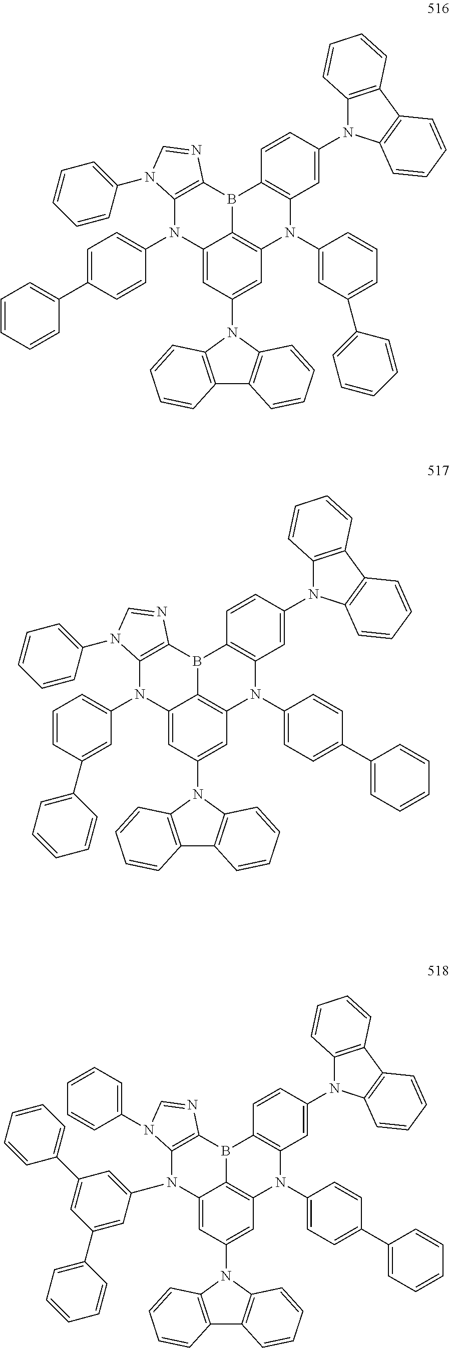 [SMILES: c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4ncn(-c5ccccc5)c43)cc2)cc1 ; c1ccc(-c2ccc(N3c4cc(-n5c6ccccc6c6ccccc65)ccc4B4c5ncn(-c6ccccc6)c5N(c5cccc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ncn(-c6ccccc6)c5N(c5cc(-c6ccccc6)cc(-c6ccccc6)c5)c5cc(-n6c7ccccc7c7ccccc76)cc3c54)cc2)cc1]

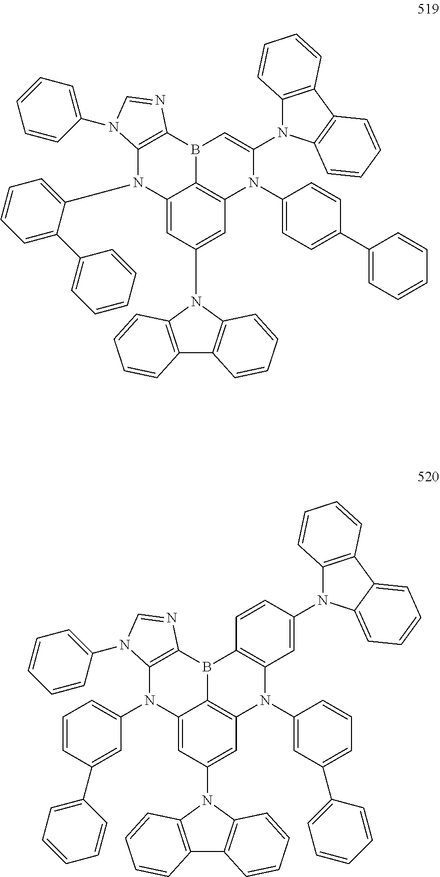 [SMILES: C1=C(n2c3ccccc3c3ccccc32)N(c2ccc(-c3ccccc3)cc2)c2cc(-n3c4ccccc4c4ccccc43)cc3c2B1c1ncn(-c2ccccc2)c1N3c1ccccc1-c1ccccc1 ; c1ccc(-c2cccc(N3c4cc(-n5c6ccccc6c6ccccc65)ccc4B4c5ncn(-c6ccccc6)c5N(c5cccc(-c6ccccc6)c5)c5cc(-n6c7ccccc7c7ccccc76)cc3c54)c2)cc1]

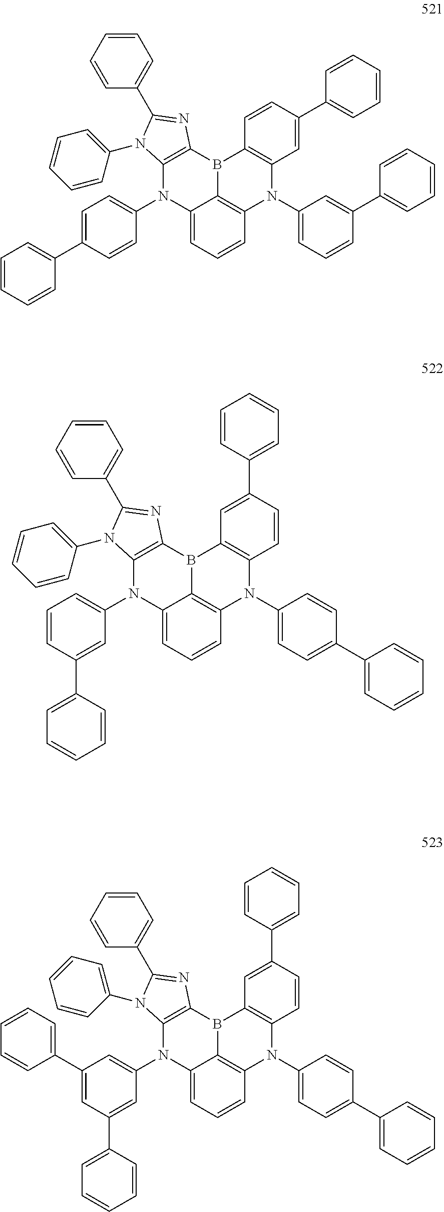 [SMILES: c1ccc(-c2ccc(N3c4cccc5c4B(c4ccc(-c6ccccc6)cc4N5c4cccc(-c5ccccc5)c4)c4nc(-c5ccccc5)n(-c5ccccc5)c43)cc2)cc1 ; c1ccc(-c2ccc(N3c4ccc(-c5ccccc5)cc4B4c5nc(-c6ccccc6)n(-c6ccccc6)c5N(c5cccc(-c6ccccc6)c5)c5cccc3c54)cc2)cc1 ; c1ccc(-c2ccc(N3c4ccc(-c5ccccc5)cc4B4c5nc(-c6ccccc6)n(-c6ccccc6)c5N(c5cc(-c6ccccc6)cc(-c6ccccc6)c5)c5cccc3c54)cc2)cc1]

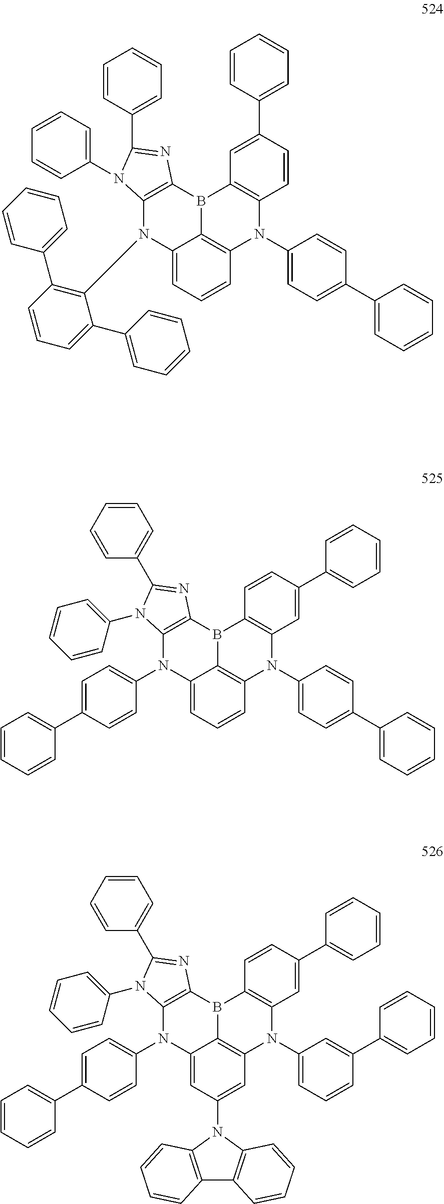 [SMILES: c1ccc(-c2ccc(N3c4ccc(-c5ccccc5)cc4B4c5nc(-c6ccccc6)n(-c6ccccc6)c5N(c5c(-c6ccccc6)cccc5-c5ccccc5)c5cccc3c54)cc2)cc1 ; c1ccc(-c2ccc(N3c4cc(-c5ccccc5)ccc4B4c5nc(-c6ccccc6)n(-c6ccccc6)c5N(c5ccc(-c6ccccc6)cc5)c5cccc3c54)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4nc(-c5ccccc5)n(-c5ccccc5)c43)cc2)cc1]

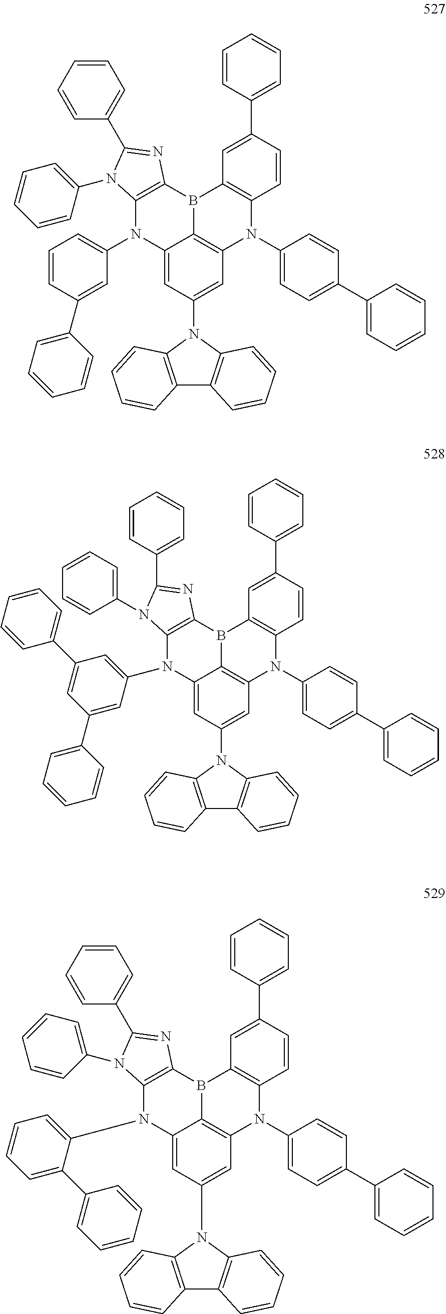 [SMILES: c1ccc(-c2ccc(N3c4ccc(-c5ccccc5)cc4B4c5nc(-c6ccccc6)n(-c6ccccc6)c5N(c5cccc(-c6ccccc6)c5)c5cc(-n6c7ccccc7c7ccccc76)cc3c54)cc2)cc1 ; c1ccc(-c2ccc(N3c4ccc(-c5ccccc5)cc4B4c5nc(-c6ccccc6)n(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4ccc(-c5ccccc5)cc4B4c5nc(-c6ccccc6)n(-c6ccccc6)c5N(c5ccccc5-c5ccccc5)c5cc(-n6c7ccccc7c7ccccc76)cc3c54)cc2)cc1]

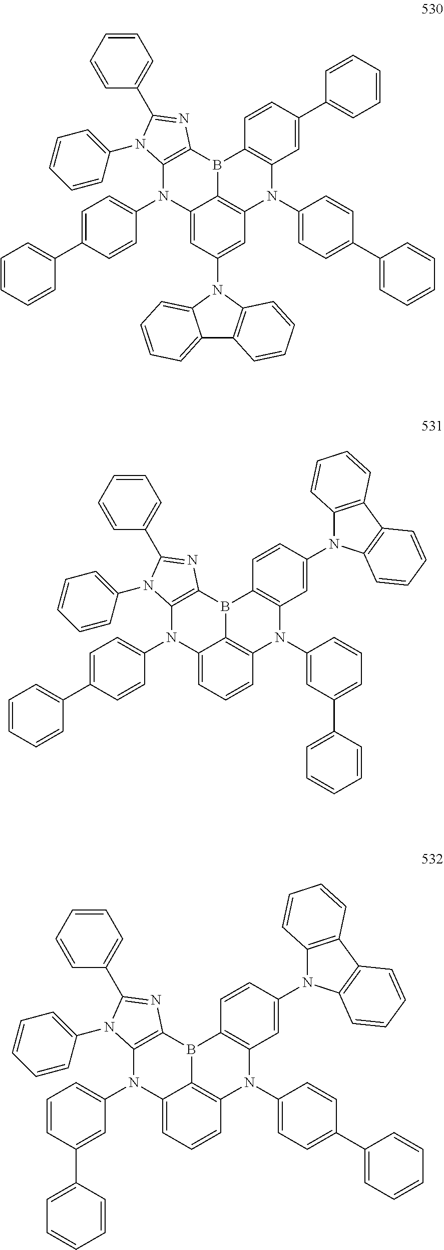 [SMILES: c1ccc(-c2ccc(N3c4cc(-c5ccccc5)ccc4B4c5nc(-c6ccccc6)n(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cccc5c4B(c4ccc(-n6c7ccccc7c7ccccc76)cc4N5c4cccc(-c5ccccc5)c4)c4nc(-c5ccccc5)n(-c5ccccc5)c43)cc2)cc1 ; c1ccc(-c2ccc(N3c4cc(-n5c6ccccc6c6ccccc65)ccc4B4c5nc(-c6ccccc6)n(-c6ccccc6)c5N(c5cccc(-c6ccccc6)c5)c5cccc3c54)cc2)cc1]

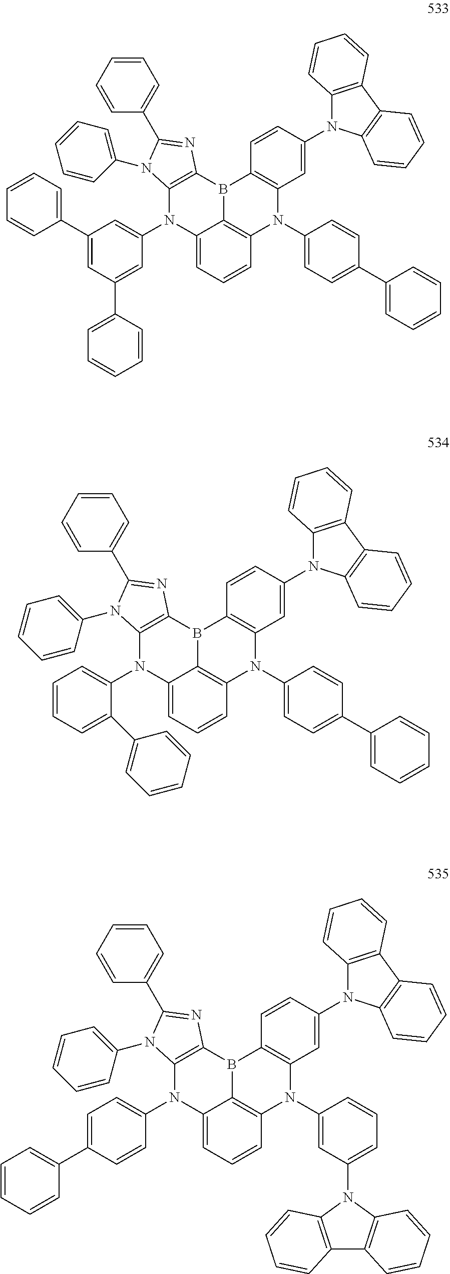 [SMILES: c1ccc(-c2ccc(N3c4cc(-n5c6ccccc6c6ccccc65)ccc4B4c5nc(-c6ccccc6)n(-c6ccccc6)c5N(c5cc(-c6ccccc6)cc(-c6ccccc6)c5)c5cccc3c54)cc2)cc1 ; c1ccc(-c2ccc(N3c4cc(-n5c6ccccc6c6ccccc65)ccc4B4c5nc(-c6ccccc6)n(-c6ccccc6)c5N(c5ccccc5-c5ccccc5)c5cccc3c54)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4nc(-c5ccccc5)n(-c5ccccc5)c43)cc2)cc1]

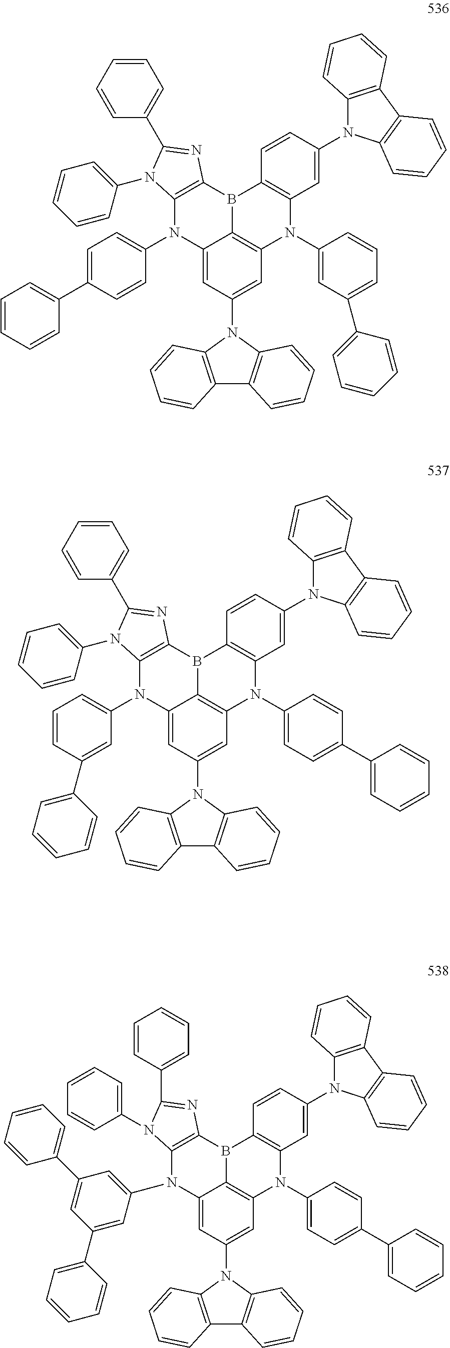 [SMILES: c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4nc(-c5ccccc5)n(-c5ccccc5)c43)cc2)cc1 ; c1ccc(-c2ccc(N3c4cc(-n5c6ccccc6c6ccccc65)ccc4B4c5nc(-c6ccccc6)n(-c6ccccc6)c5N(c5cccc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nc(-c6ccccc6)n(-c6ccccc6)c5N(c5cc(-c6ccccc6)cc(-c6ccccc6)c5)c5cc(-n6c7ccccc7c7ccccc76)cc3c54)cc2)cc1]

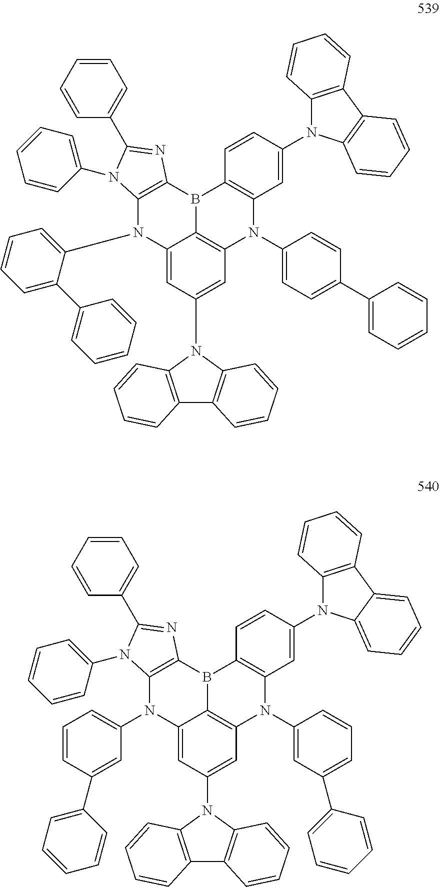 [SMILES: c1ccc(-c2ccc(N3c4cc(-n5c6ccccc6c6ccccc65)ccc4B4c5nc(-c6ccccc6)n(-c6ccccc6)c5N(c5ccccc5-c5ccccc5)c5cc(-n6c7ccccc7c7ccccc76)cc3c54)cc2)cc1 ; c1ccc(-c2cccc(N3c4cc(-n5c6ccccc6c6ccccc65)ccc4B4c5nc(-c6ccccc6)n(-c6ccccc6)c5N(c5cccc(-c6ccccc6)c5)c5cc(-n6c7ccccc7c7ccccc76)cc3c54)c2)cc1]

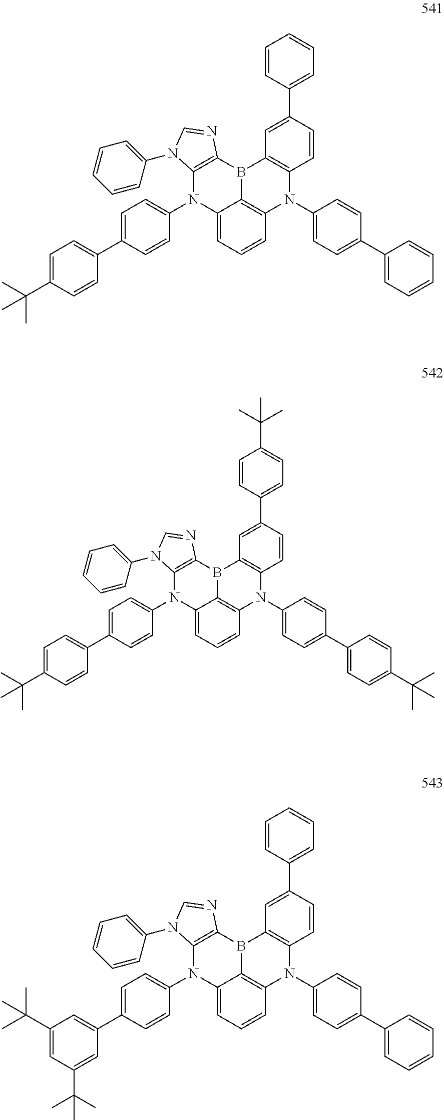

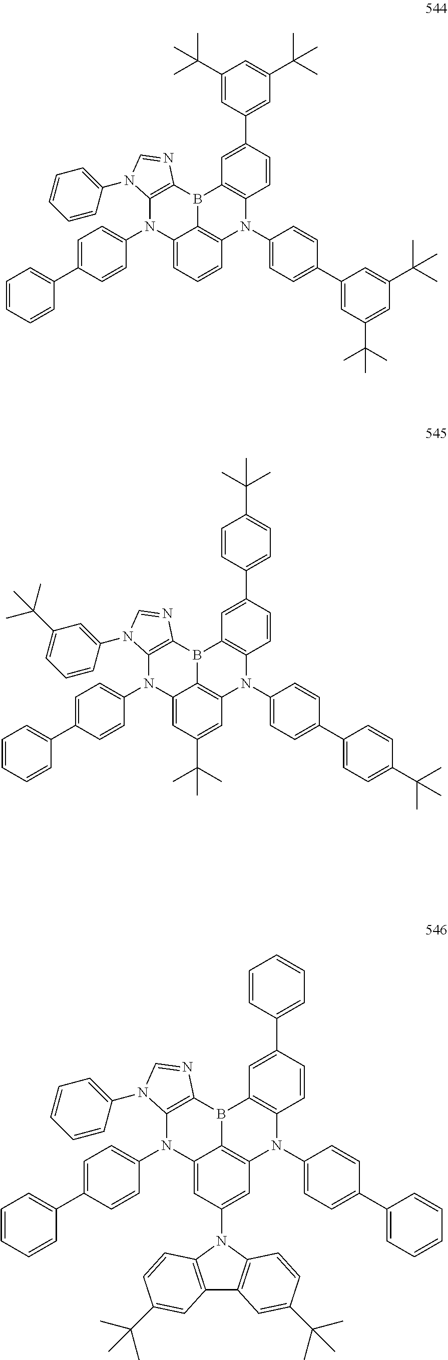 [SMILES: CC(C)(C)c1cc(-c2ccc(N3c4ccc(-c5cc(C(C)(C)C)cc(C(C)(C)C)c5)cc4B4c5ncn(-c6ccccc6)c5N(c5ccc(-c6ccccc6)cc5)c5cccc3c54)cc2)cc(C(C)(C)C)c1 ; CC(C)(C)c1ccc(-c2ccc(N3c4ccc(-c5ccc(C(C)(C)C)cc5)cc4B4c5ncn(-c6cccc(C(C)(C)C)c6)c5N(c5ccc(-c6ccccc6)cc5)c5cc(C(C)(C)C)cc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(ncn1-c1ccccc1)B3c1cc(-c3ccccc3)ccc1N2c1ccc(-c2ccccc2)cc1]

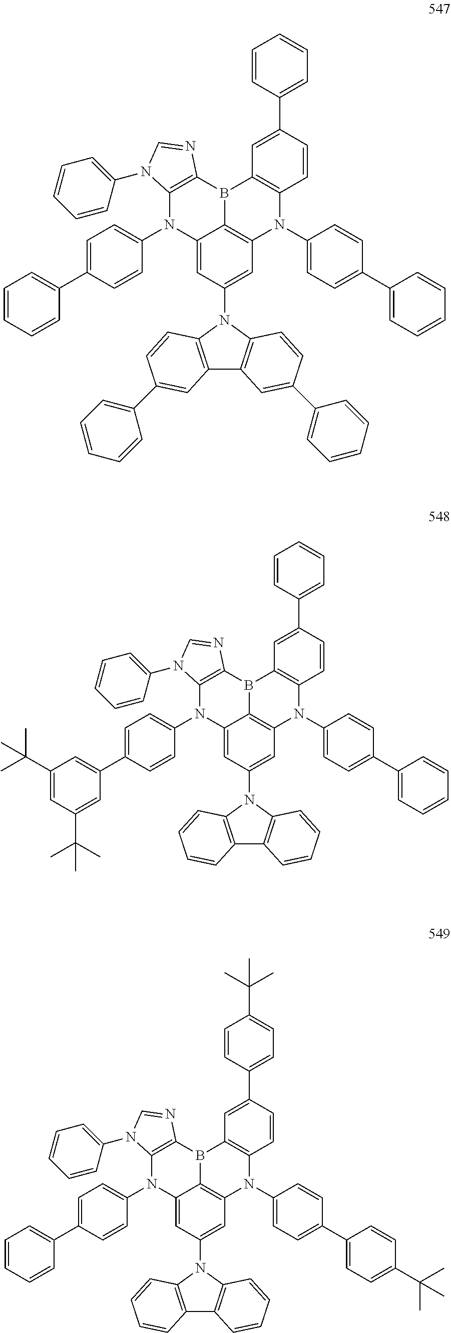 [SMILES: c1ccc(-c2ccc(N3c4ccc(-c5ccccc5)cc4B4c5ncn(-c6ccccc6)c5N(c5ccc(-c6ccccc6)cc5)c5cc(-n6c7ccc(-c8ccccc8)cc7c7cc(-c8ccccc8)ccc76)cc3c54)cc2)cc1 ; CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4ncn(-c5ccccc5)c43)cc2)cc(C(C)(C)C)c1 ; CC(C)(C)c1ccc(-c2ccc(N3c4ccc(-c5ccc(C(C)(C)C)cc5)cc4B4c5ncn(-c6ccccc6)c5N(c5ccc(-c6ccccc6)cc5)c5cc(-n6c7ccccc7c7ccccc76)cc3c54)cc2)cc1]

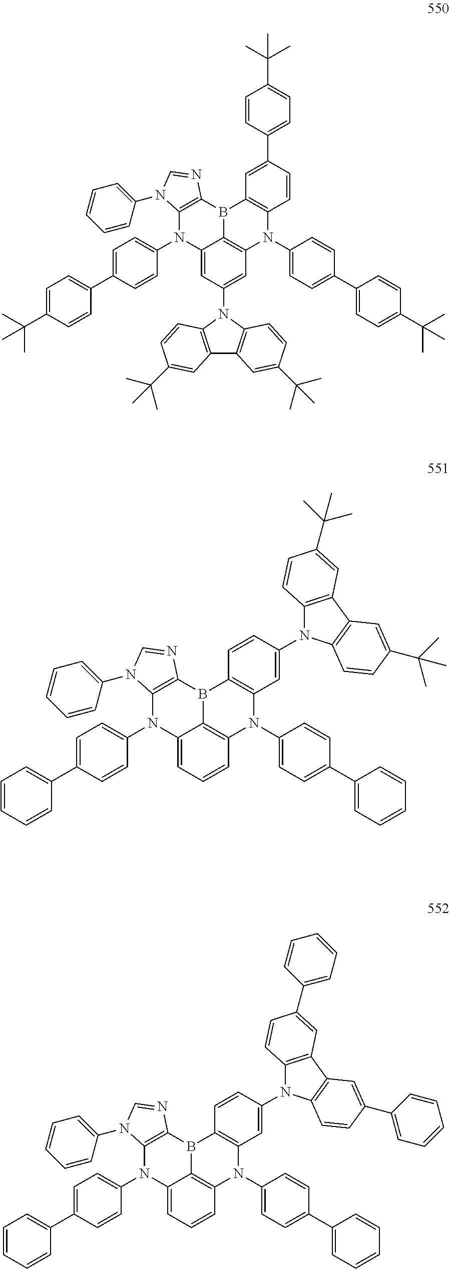 [SMILES: CC(C)(C)c1ccc(-c2ccc(N3c4ccc(-c5ccc(C(C)(C)C)cc5)cc4B4c5ncn(-c6ccccc6)c5N(c5ccc(-c6ccc(C(C)(C)C)cc6)cc5)c5cc(-n6c7ccc(C(C)(C)C)cc7c7cc(C(C)(C)C)ccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ncn(-c2ccccc2)c1N3c1ccc(-c2ccccc2)cc1 ; c1ccc(-c2ccc(N3c4cc(-n5c6ccc(-c7ccccc7)cc6c6cc(-c7ccccc7)ccc65)ccc4B4c5ncn(-c6ccccc6)c5N(c5ccc(-c6ccccc6)cc5)c5cccc3c54)cc2)cc1]

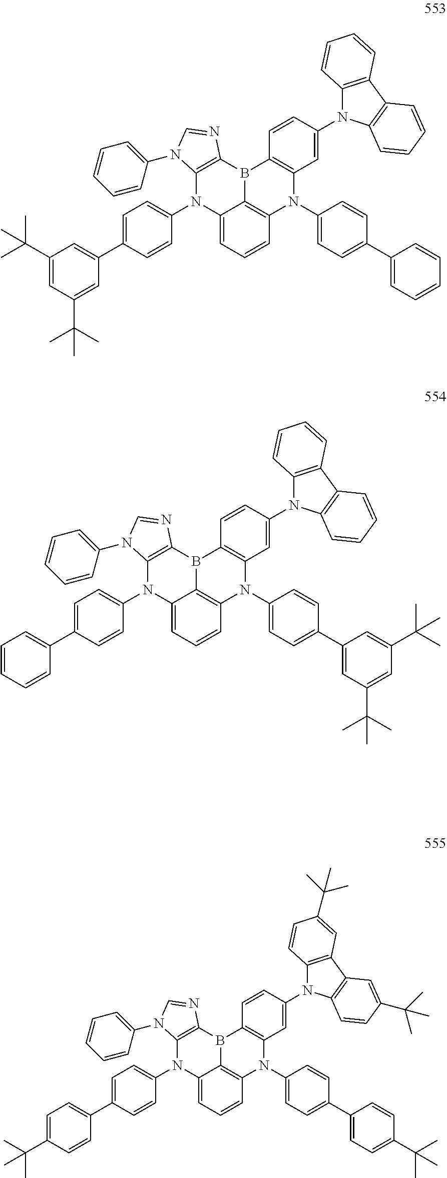

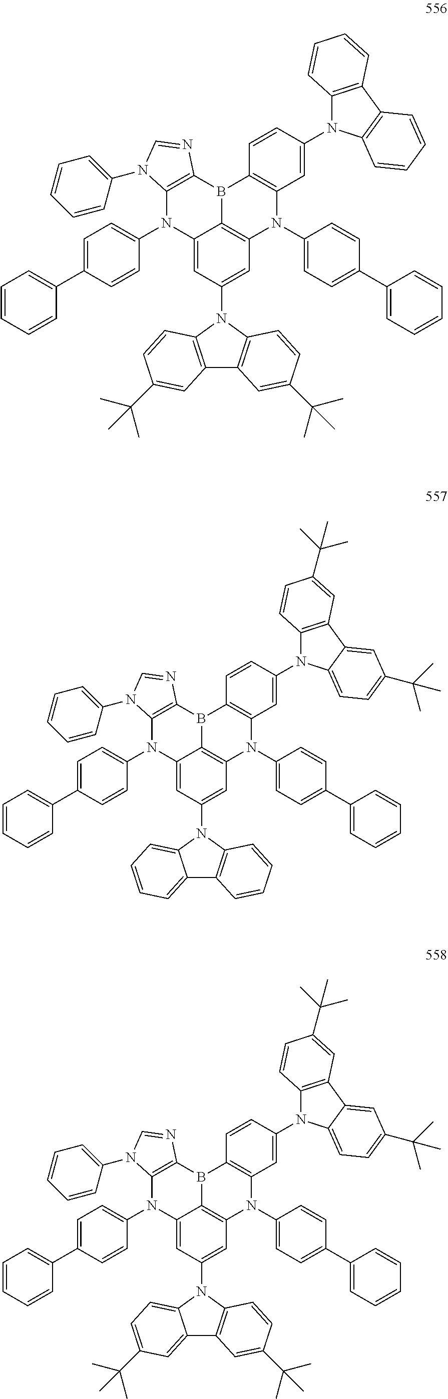 [SMILES: CC(C)(C)c1ccc2c(c1)c1cc(C(C)(C)C)ccc1n2-c1cc2c3c(c1)N(c1ccc(-c4ccccc4)cc1)c1c(ncn1-c1ccccc1)B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ncn(-c2ccccc2)c1N3c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccc(C(C)(C)C)cc4c4cc(C(C)(C)C)ccc43)cc3c1B2c1ncn(-c2ccccc2)c1N3c1ccc(-c2ccccc2)cc1]

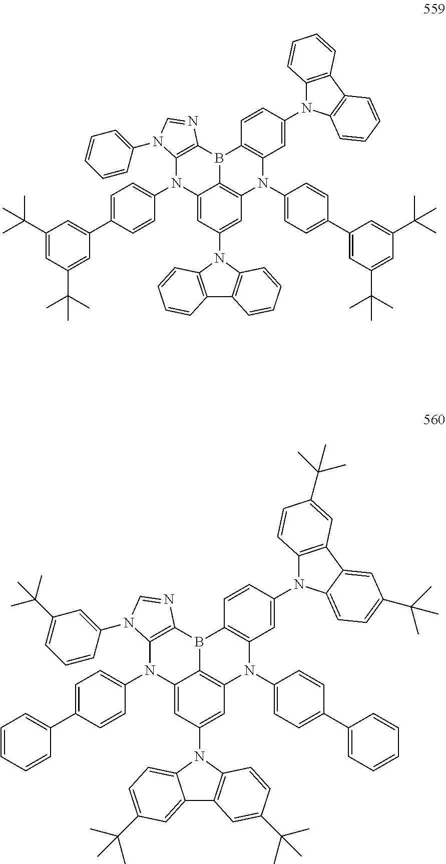 [SMILES: CC(C)(C)c1cc(-c2ccc(N3c4cc(-n5c6ccccc6c6ccccc65)ccc4B4c5ncn(-c6ccccc6)c5N(c5ccc(-c6cc(C(C)(C)C)cc(C(C)(C)C)c6)cc5)c5cc(-n6c7ccccc7c7ccccc76)cc3c54)cc2)cc(C(C)(C)C)c1 ; CC(C)(C)c1cccc(-n2cnc3c2N(c2ccc(-c4ccccc4)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccccc3)cc2)c1]

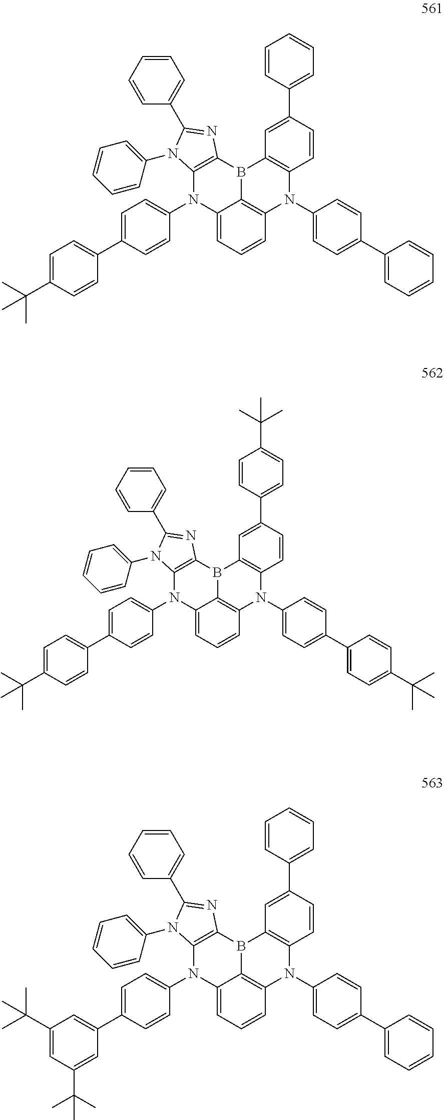

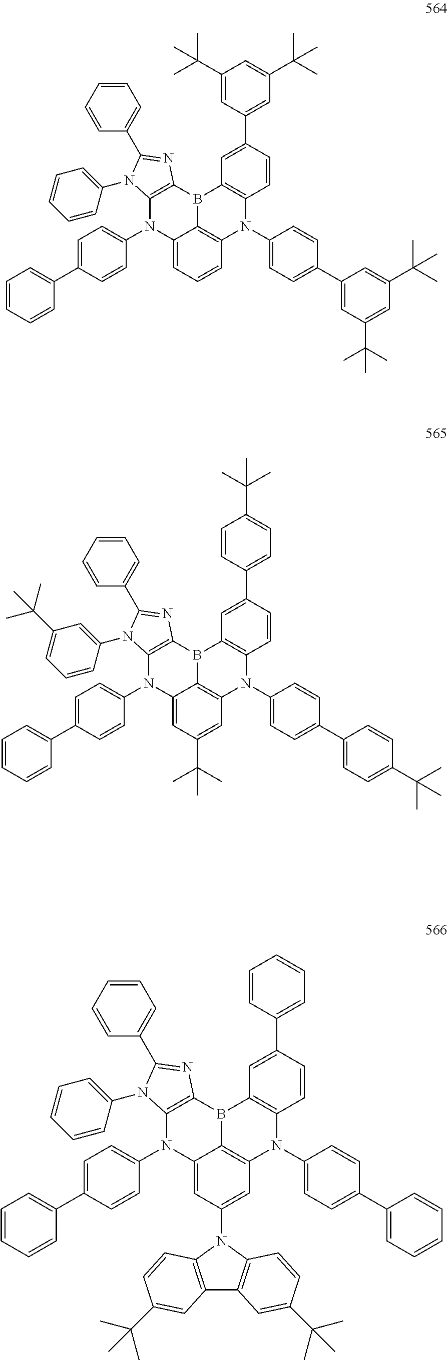 [SMILES: CC(C)(C)c1cc(-c2ccc(N3c4ccc(-c5cc(C(C)(C)C)cc(C(C)(C)C)c5)cc4B4c5nc(-c6ccccc6)n(-c6ccccc6)c5N(c5ccc(-c6ccccc6)cc5)c5cccc3c54)cc2)cc(C(C)(C)C)c1 ; CC(C)(C)c1ccc(-c2ccc(N3c4ccc(-c5ccc(C(C)(C)C)cc5)cc4B4c5nc(-c6ccccc6)n(-c6cccc(C(C)(C)C)c6)c5N(c5ccc(-c6ccccc6)cc5)c5cc(C(C)(C)C)cc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(nc(-c4ccccc4)n1-c1ccccc1)B3c1cc(-c3ccccc3)ccc1N2c1ccc(-c2ccccc2)cc1]

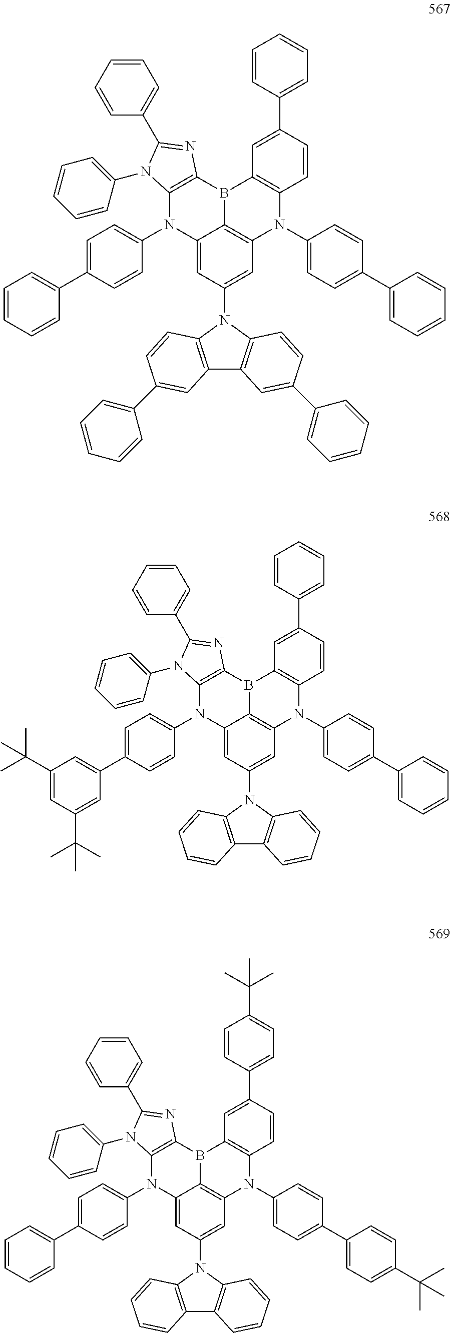 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccc(-c8ccccc8)cc7c7cc(-c8ccccc8)ccc76)cc3c54)cc2)cc1 ; CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4nc(-c5ccccc5)n(-c5ccccc5)c43)cc2)cc(C(C)(C)C)c1 ; CC(C)(C)c1ccc(-c2ccc(N3c4ccc(-c5ccc(C(C)(C)C)cc5)cc4B4c5nc(-c6ccccc6)n(-c6ccccc6)c5N(c5ccc(-c6ccccc6)cc5)c5cc(-n6c7ccccc7c7ccccc76)cc3c54)cc2)cc1]

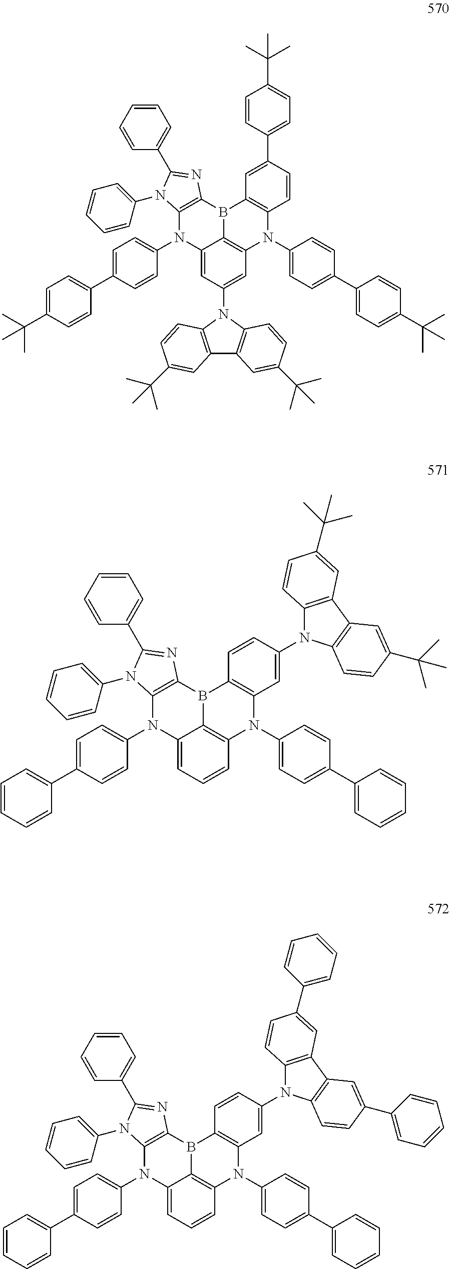 [SMILES: CC(C)(C)c1ccc(-c2ccc(N3c4ccc(-c5ccc(C(C)(C)C)cc5)cc4B4c5nc(-c6ccccc6)n(-c6ccccc6)c5N(c5ccc(-c6ccc(C(C)(C)C)cc6)cc5)c5cc(-n6c7ccc(C(C)(C)C)cc7c7cc(C(C)(C)C)ccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nc(-c2ccccc2)n(-c2ccccc2)c1N3c1ccc(-c2ccccc2)cc1 ; c1ccc(-c2ccc(N3c4cc(-n5c6ccc(-c7ccccc7)cc6c6cc(-c7ccccc7)ccc65)ccc4B4c5nc(-c6ccccc6)n(-c6ccccc6)c5N(c5ccc(-c6ccccc6)cc5)c5cccc3c54)cc2)cc1]

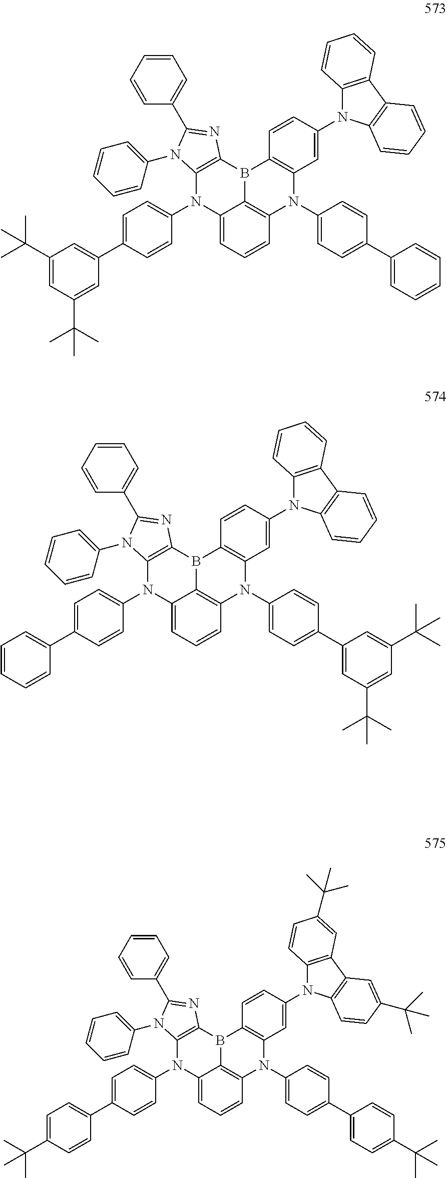

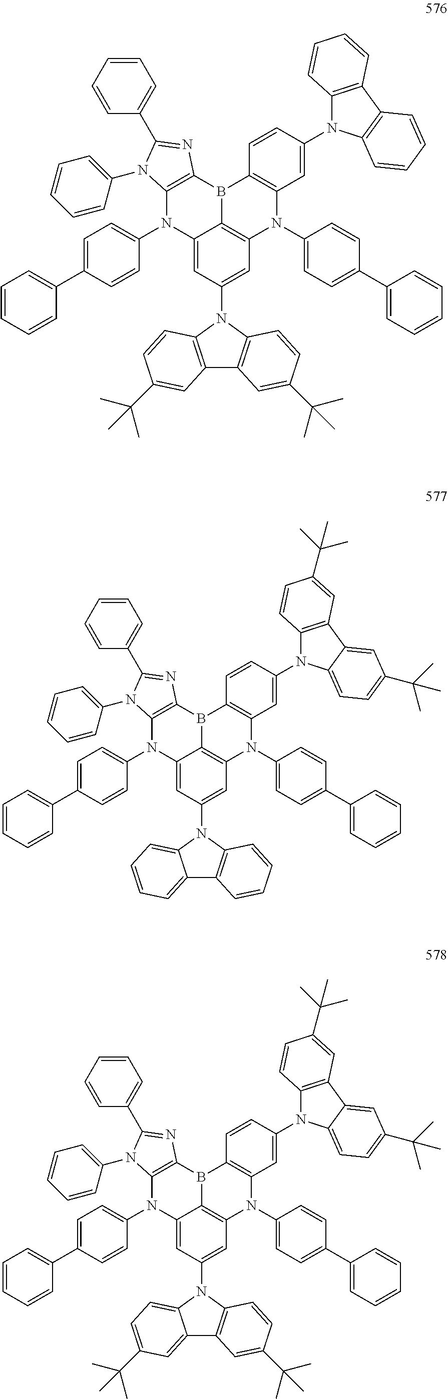 [SMILES: CC(C)(C)c1ccc2c(c1)c1cc(C(C)(C)C)ccc1n2-c1cc2c3c(c1)N(c1ccc(-c4ccccc4)cc1)c1c(nc(-c4ccccc4)n1-c1ccccc1)B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nc(-c2ccccc2)n(-c2ccccc2)c1N3c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccc(C(C)(C)C)cc4c4cc(C(C)(C)C)ccc43)cc3c1B2c1nc(-c2ccccc2)n(-c2ccccc2)c1N3c1ccc(-c2ccccc2)cc1]

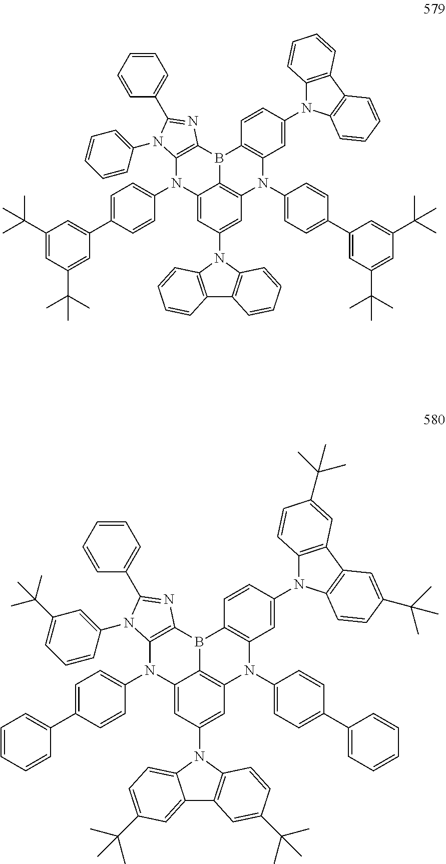 [SMILES: CC(C)(C)c1cc(-c2ccc(N3c4cc(-n5c6ccccc6c6ccccc65)ccc4B4c5nc(-c6ccccc6)n(-c6ccccc6)c5N(c5ccc(-c6cc(C(C)(C)C)cc(C(C)(C)C)c6)cc5)c5cc(-n6c7ccccc7c7ccccc76)cc3c54)cc2)cc(C(C)(C)C)c1 ; CC(C)(C)c1cccc(-n2c(-c3ccccc3)nc3c2N(c2ccc(-c4ccccc4)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccccc3)cc2)c1]

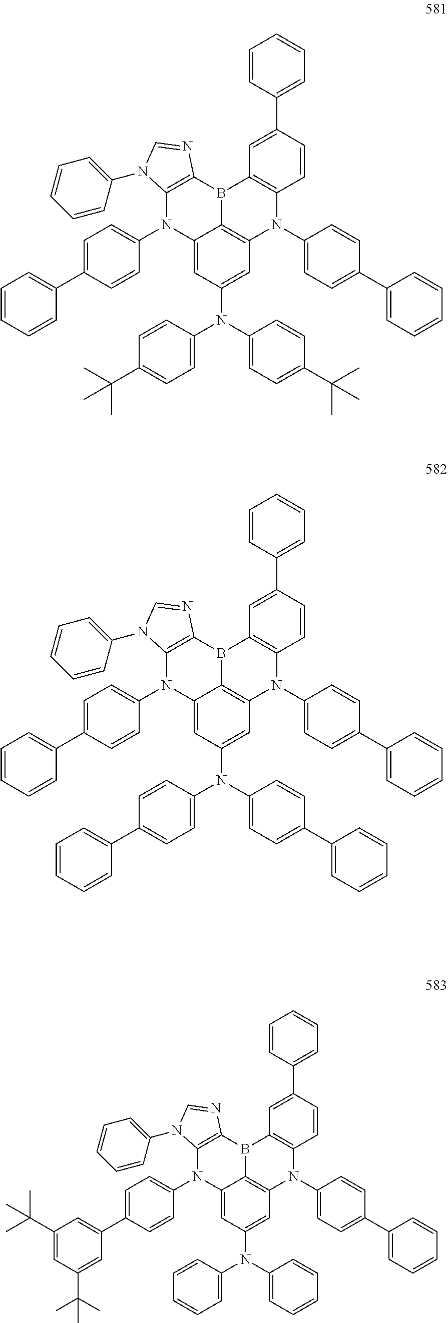

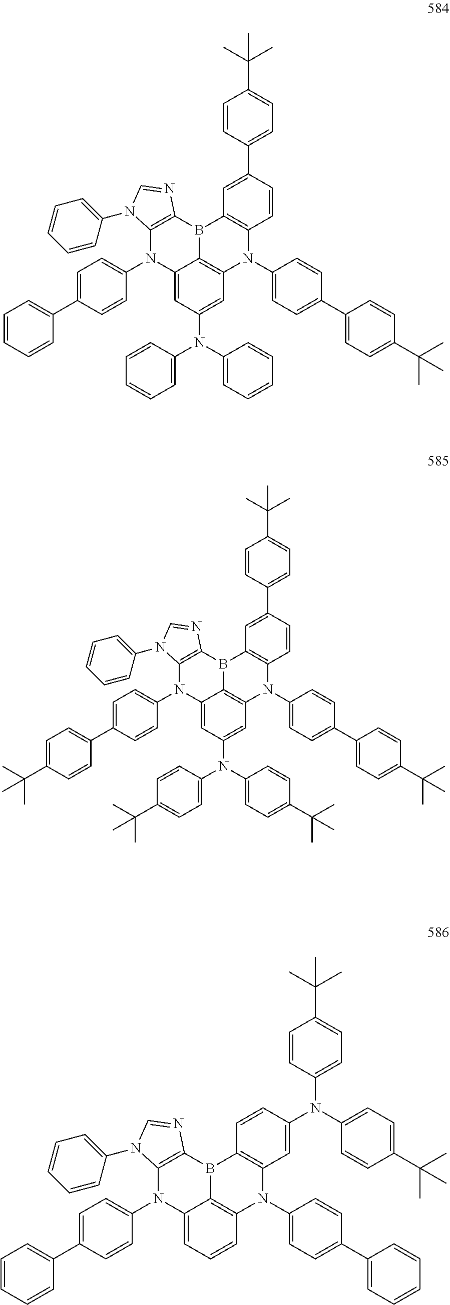

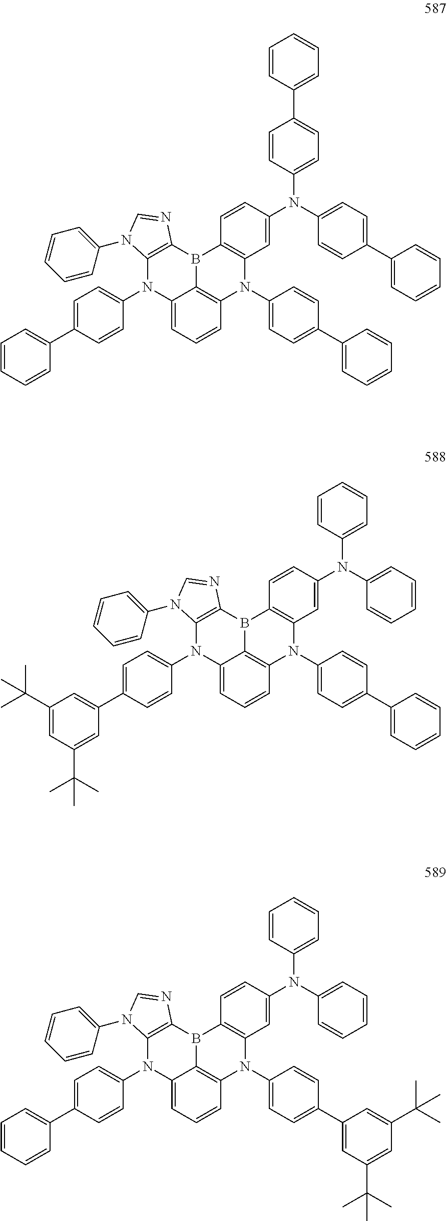 [SMILES: c1ccc(-c2ccc(N(c3ccc(-c4ccccc4)cc3)c3ccc4c(c3)N(c3ccc(-c5ccccc5)cc3)c3cccc5c3B4c3ncn(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4ncn(-c5ccccc5)c43)cc2)cc(C(C)(C)C)c1 ; CC(C)(C)c1cc(-c2ccc(N3c4cc(N(c5ccccc5)c5ccccc5)ccc4B4c5ncn(-c6ccccc6)c5N(c5ccc(-c6ccccc6)cc5)c5cccc3c54)cc2)cc(C(C)(C)C)c1]

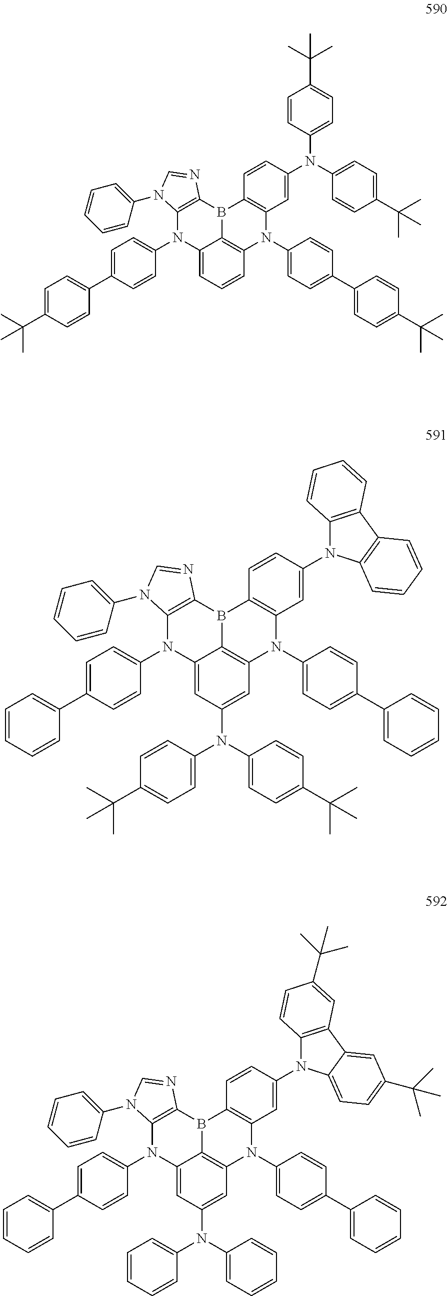 [SMILES: CC(C)(C)c1ccc(-c2ccc(N3c4cc(N(c5ccc(C(C)(C)C)cc5)c5ccc(C(C)(C)C)cc5)ccc4B4c5ncn(-c6ccccc6)c5N(c5ccc(-c6ccc(C(C)(C)C)cc6)cc5)c5cccc3c54)cc2)cc1 ; CC(C)(C)c1ccc(N(c2ccc(C(C)(C)C)cc2)c2cc3c4c(c2)N(c2ccc(-c5ccccc5)cc2)c2c(ncn2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ncn(-c2ccccc2)c1N3c1ccc(-c2ccccc2)cc1]

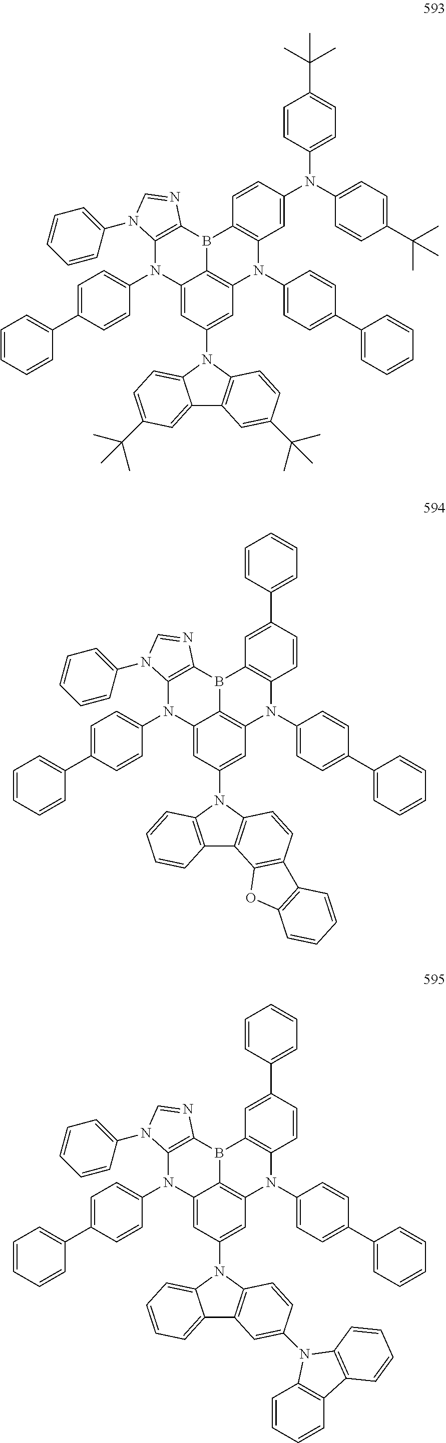 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2ncn(-c3ccccc3)c2N4c2ccc(-c3ccccc3)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-n6c7ccccc7c7c8oc9ccccc9c8ccc76)cc3c54)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-n6c7ccccc7c7cc(-n8c9ccccc9c9ccccc98)ccc76)cc3c54)cc2)cc1]

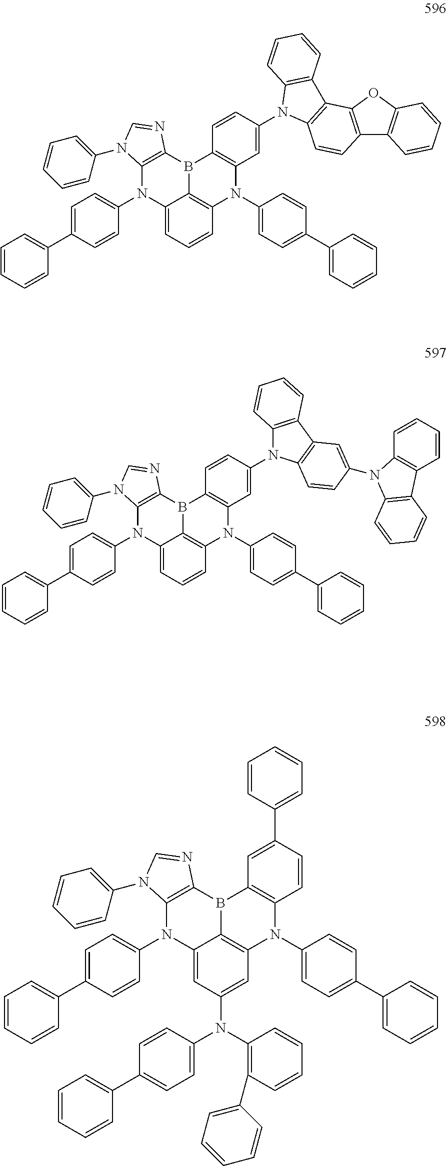 [SMILES: c1ccc(-c2ccc(N3c4cc(-n5c6ccccc6c6c7oc8ccccc8c7ccc65)ccc4B4c5ncn(-c6ccccc6)c5N(c5ccc(-c6ccccc6)cc5)c5cccc3c54)cc2)cc1 ; c1ccc(-c2ccc(N3c4cc(-n5c6ccccc6c6cc(-n7c8ccccc8c8ccccc87)ccc65)ccc4B4c5ncn(-c6ccccc6)c5N(c5ccc(-c6ccccc6)cc5)c5cccc3c54)cc2)cc1 ; c1ccc(-c2ccc(N(c3cc4c5c(c3)N(c3ccc(-c6ccccc6)cc3)c3c(ncn3-c3ccccc3)B5c3cc(-c5ccccc5)ccc3N4c3ccc(-c4ccccc4)cc3)c3ccccc3-c3ccccc3)cc2)cc1]

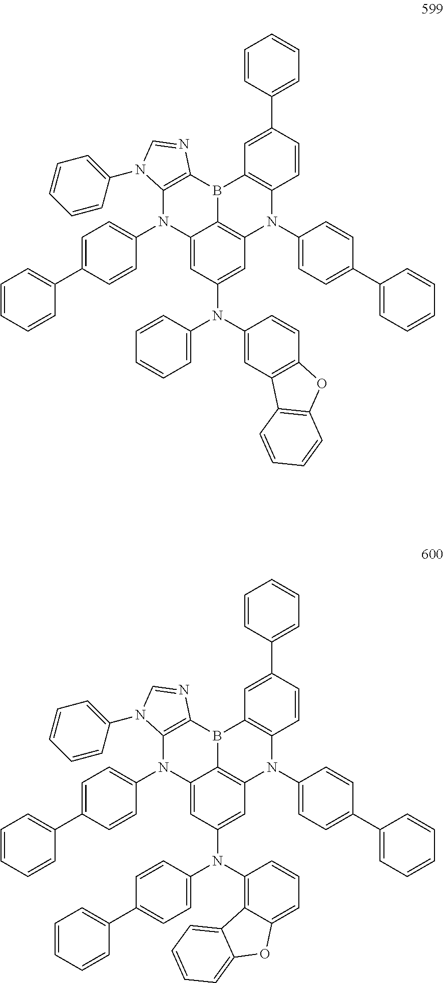 [SMILES: c1ccc(-c2ccc(N3c4ccc(-c5ccccc5)cc4B4c5ncn(-c6ccccc6)c5N(c5ccc(-c6ccccc6)cc5)c5cc(N(c6ccccc6)c6ccc7oc8ccccc8c7c6)cc3c54)cc2)cc1 ; c1ccc(-c2ccc(N3c4ccc(-c5ccccc5)cc4B4c5ncn(-c6ccccc6)c5N(c5ccc(-c6ccccc6)cc5)c5cc(N(c6ccc(-c7ccccc7)cc6)c6cccc7oc8ccccc8c67)cc3c54)cc2)cc1]

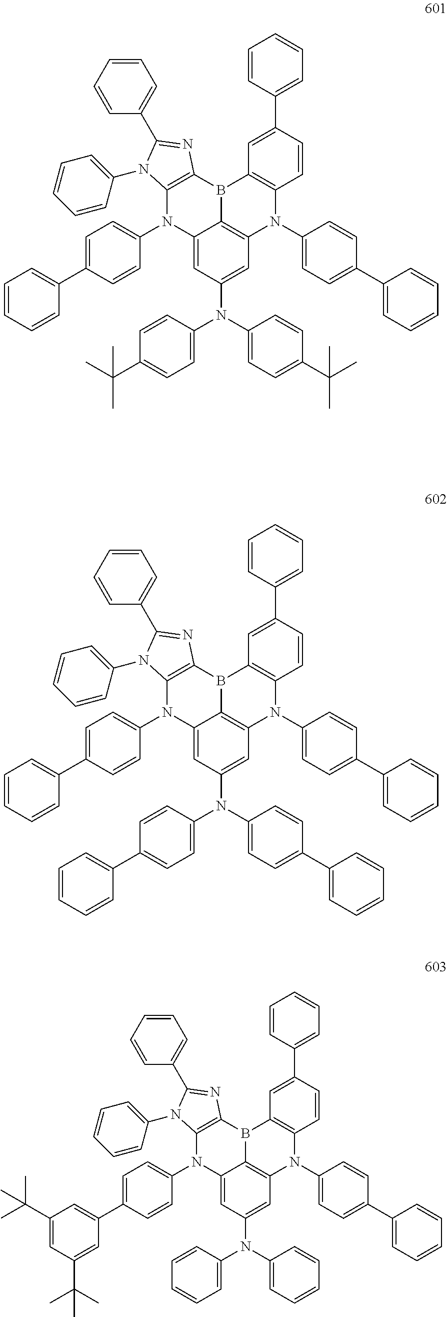 [SMILES: CC(C)(C)c1ccc(N(c2ccc(C(C)(C)C)cc2)c2cc3c4c(c2)N(c2ccc(-c5ccccc5)cc2)c2c(nc(-c5ccccc5)n2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(nc(-c6ccccc6)n3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4nc(-c5ccccc5)n(-c5ccccc5)c43)cc2)cc(C(C)(C)C)c1]

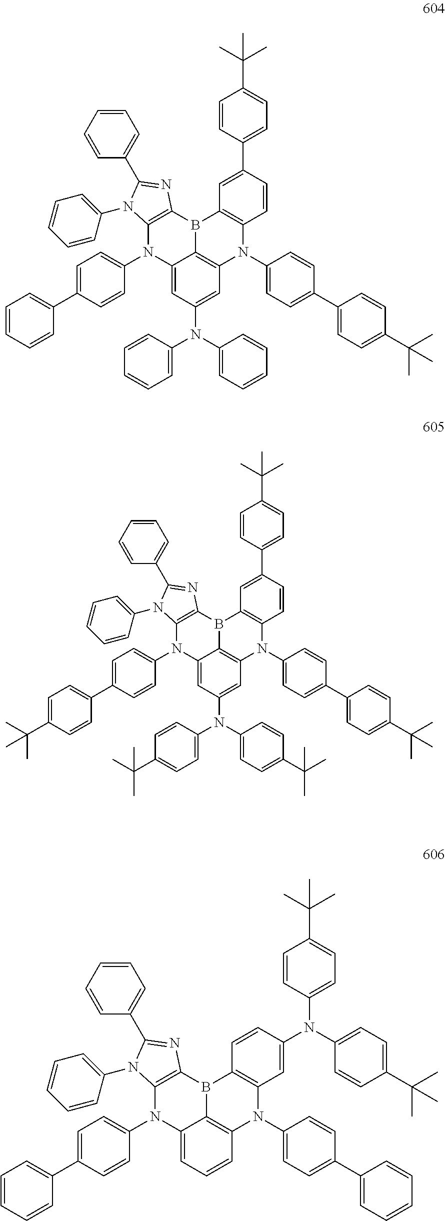

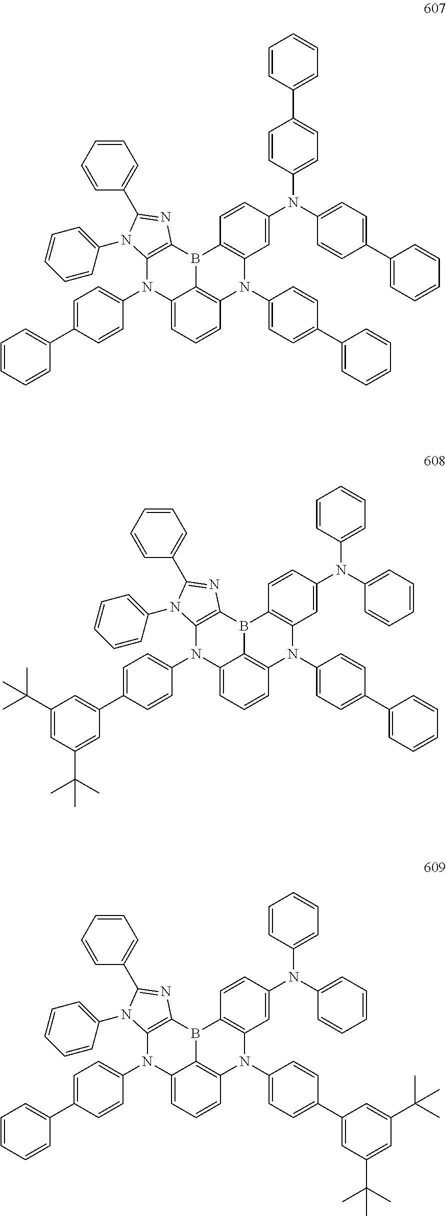 [SMILES: c1ccc(-c2ccc(N(c3ccc(-c4ccccc4)cc3)c3ccc4c(c3)N(c3ccc(-c5ccccc5)cc3)c3cccc5c3B4c3nc(-c4ccccc4)n(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4nc(-c5ccccc5)n(-c5ccccc5)c43)cc2)cc(C(C)(C)C)c1 ; CC(C)(C)c1cc(-c2ccc(N3c4cc(N(c5ccccc5)c5ccccc5)ccc4B4c5nc(-c6ccccc6)n(-c6ccccc6)c5N(c5ccc(-c6ccccc6)cc5)c5cccc3c54)cc2)cc(C(C)(C)C)c1]

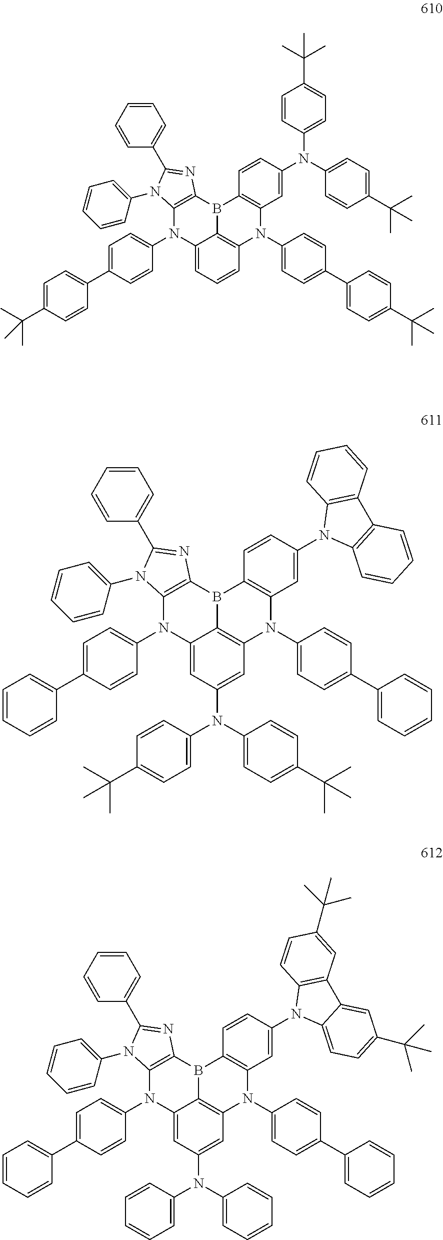 [SMILES: CC(C)(C)c1ccc(-c2ccc(N3c4cc(N(c5ccc(C(C)(C)C)cc5)c5ccc(C(C)(C)C)cc5)ccc4B4c5nc(-c6ccccc6)n(-c6ccccc6)c5N(c5ccc(-c6ccc(C(C)(C)C)cc6)cc5)c5cccc3c54)cc2)cc1 ; CC(C)(C)c1ccc(N(c2ccc(C(C)(C)C)cc2)c2cc3c4c(c2)N(c2ccc(-c5ccccc5)cc2)c2c(nc(-c5ccccc5)n2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1nc(-c2ccccc2)n(-c2ccccc2)c1N3c1ccc(-c2ccccc2)cc1]

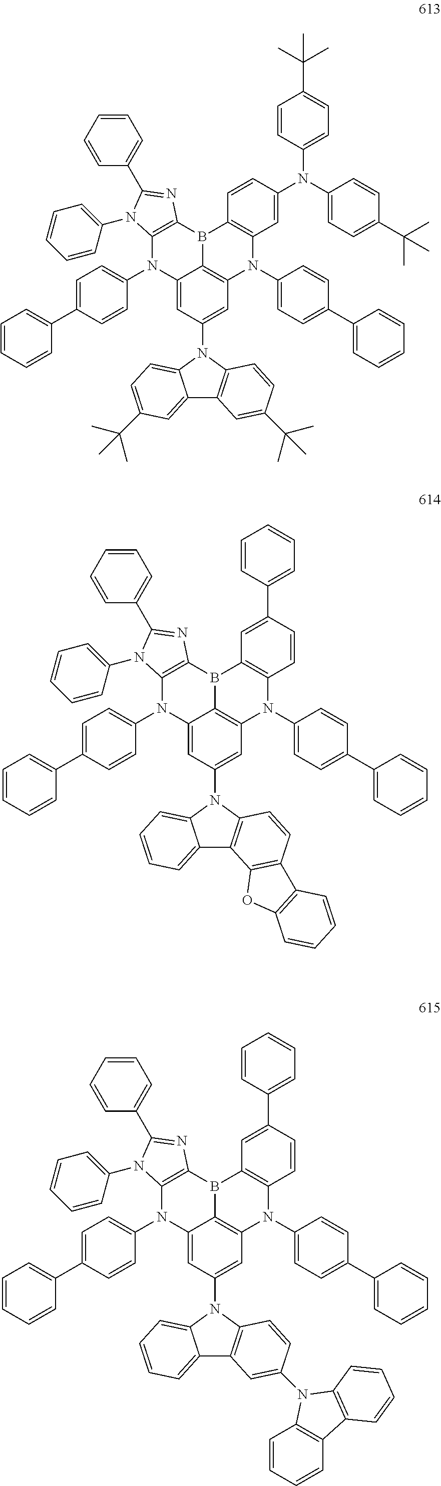 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2nc(-c3ccccc3)n(-c3ccccc3)c2N4c2ccc(-c3ccccc3)cc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7c8oc9ccccc9c8ccc76)cc3c54)cc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7cc(-n8c9ccccc9c9ccccc98)ccc76)cc3c54)cc2)cc1]

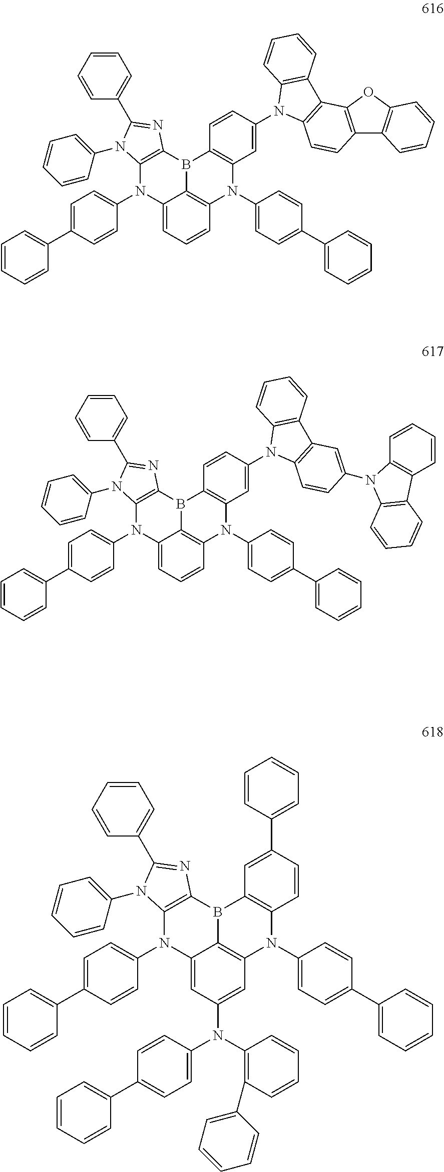 [SMILES: c1ccc(-c2ccc(N3c4cc(-n5c6ccccc6c6c7oc8ccccc8c7ccc65)ccc4B4c5nc(-c6ccccc6)n(-c6ccccc6)c5N(c5ccc(-c6ccccc6)cc5)c5cccc3c54)cc2)cc1 ; c1ccc(-c2ccc(N3c4cc(-n5c6ccccc6c6cc(-n7c8ccccc8c8ccccc87)ccc65)ccc4B4c5nc(-c6ccccc6)n(-c6ccccc6)c5N(c5ccc(-c6ccccc6)cc5)c5cccc3c54)cc2)cc1 ; c1ccc(-c2ccc(N(c3cc4c5c(c3)N(c3ccc(-c6ccccc6)cc3)c3c(nc(-c6ccccc6)n3-c3ccccc3)B5c3cc(-c5ccccc5)ccc3N4c3ccc(-c4ccccc4)cc3)c3ccccc3-c3ccccc3)cc2)cc1]

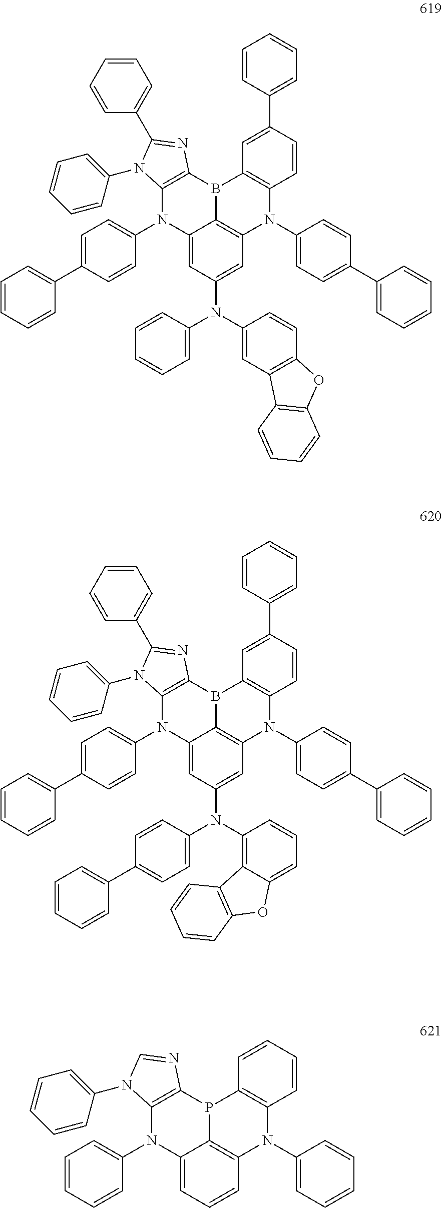 [SMILES: c1ccc(-c2ccc(N3c4ccc(-c5ccccc5)cc4B4c5nc(-c6ccccc6)n(-c6ccccc6)c5N(c5ccc(-c6ccccc6)cc5)c5cc(N(c6ccccc6)c6ccc7oc8ccccc8c7c6)cc3c54)cc2)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(N(c6ccc(-c7ccccc7)cc6)c6cccc7oc8ccccc8c67)cc3c54)cc2)cc1 ; c1ccc(N2c3ccccc3P3c4ncn(-c5ccccc5)c4N(c4ccccc4)c4cccc2c43)cc1]

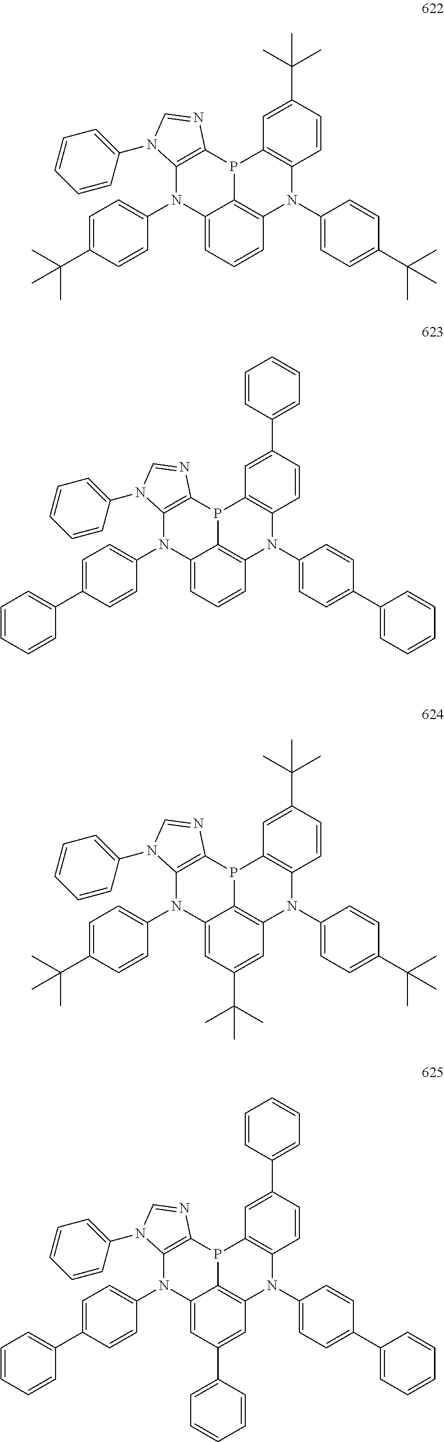

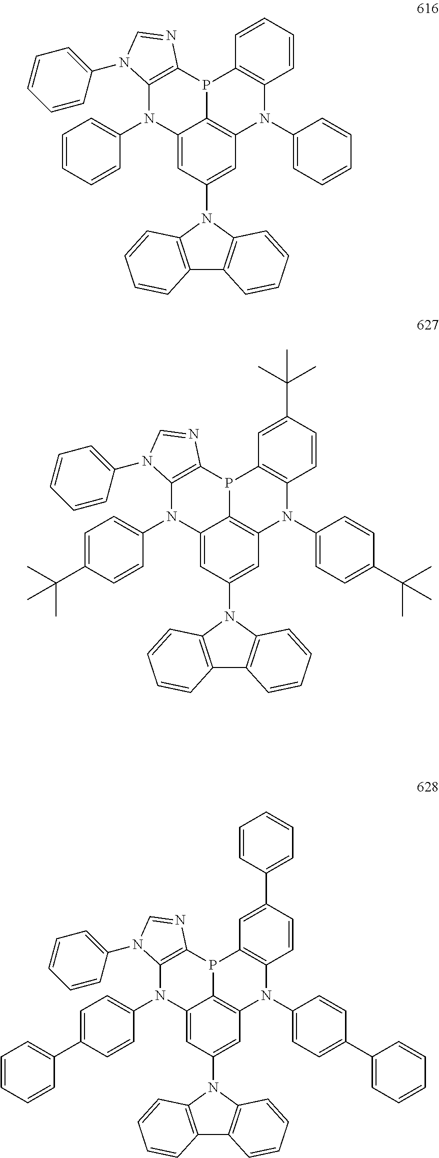 [SMILES: c1ccc(N2c3ccccc3P3c4ncn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3P3c4ncn(-c5ccccc5)c4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4P4c5ncn(-c6ccccc6)c5N(c5ccc(-c6ccccc6)cc5)c5cc(-n6c7ccccc7c7ccccc76)cc3c54)cc2)cc1]

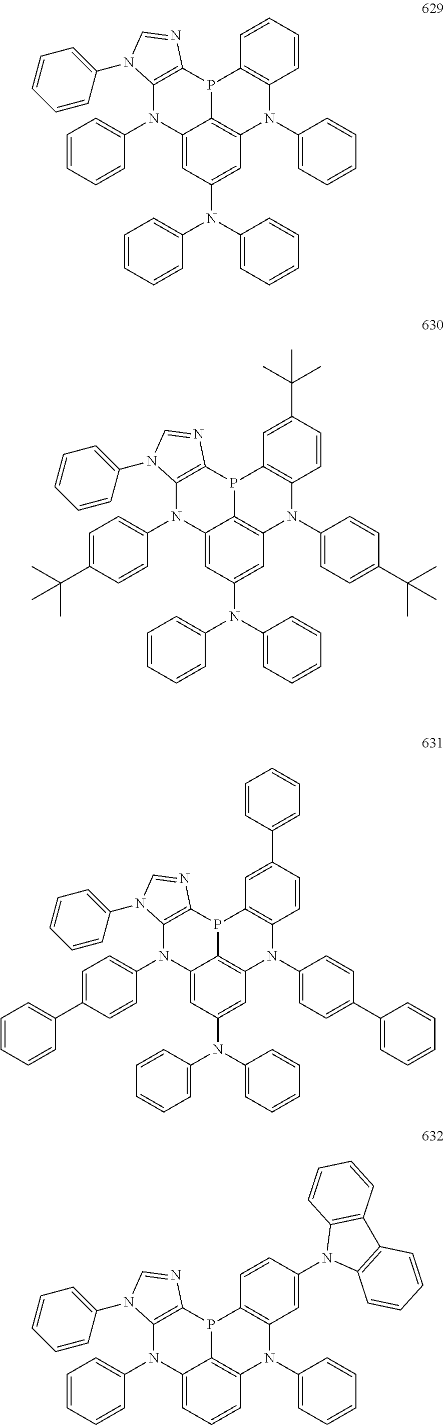 [SMILES: c1ccc(N(c2ccccc2)c2cc3c4c(c2)N(c2ccccc2)c2c(ncn2-c2ccccc2)P4c2ccccc2N3c2ccccc2)cc1 ; CC(C)(C)c1ccc(N2c3ccc(C(C)(C)C)cc3P3c4ncn(-c5ccccc5)c4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4P4c5ncn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3P3c4ncn(-c5ccccc5)c4N(c4ccccc4)c4cccc2c43)cc1]

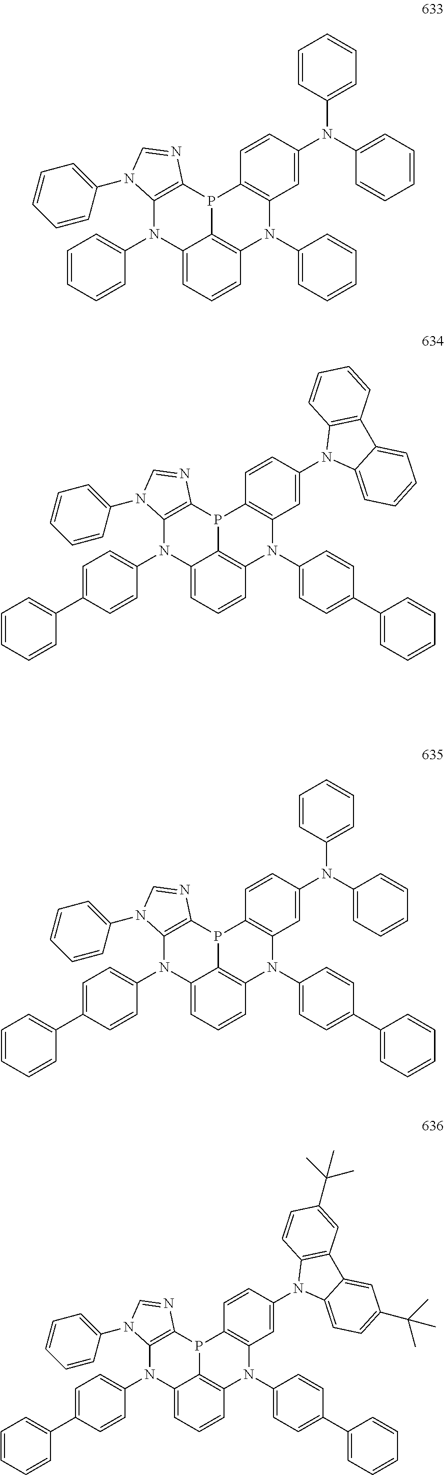 [SMILES: c1ccc(N(c2ccccc2)c2ccc3c(c2)N(c2ccccc2)c2cccc4c2P3c2ncn(-c3ccccc3)c2N4c2ccccc2)cc1 ; c1ccc(-c2ccc(N3c4cc(-n5c6ccccc6c6ccccc65)ccc4P4c5ncn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4P4c5ncn(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1ncn(-c2ccccc2)c1N3c1ccc(-c2ccccc2)cc1]

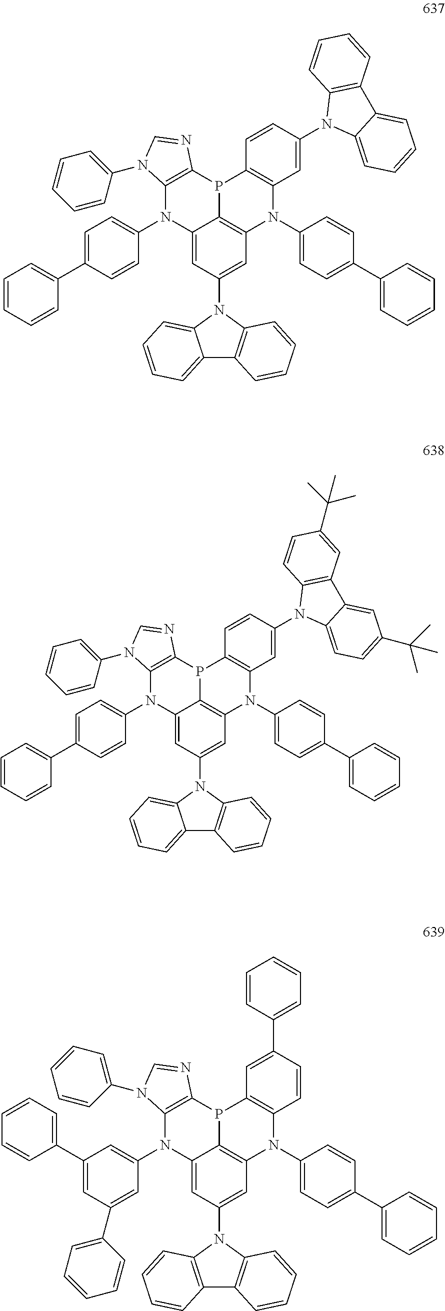 [SMILES: c1ccc(-c2ccc(N3c4cc(-n5c6ccccc6c6ccccc65)ccc4P4c5ncn(-c6ccccc6)c5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1ncn(-c2ccccc2)c1N3c1ccc(-c2ccccc2)cc1 ; c1ccc(-c2ccc(N3c4ccc(-c5ccccc5)cc4P4c5ncn(-c6ccccc6)c5N(c5cc(-c6ccccc6)cc(-c6ccccc6)c5)c5cc(-n6c7ccccc7c7ccccc76)cc3c54)cc2)cc1]

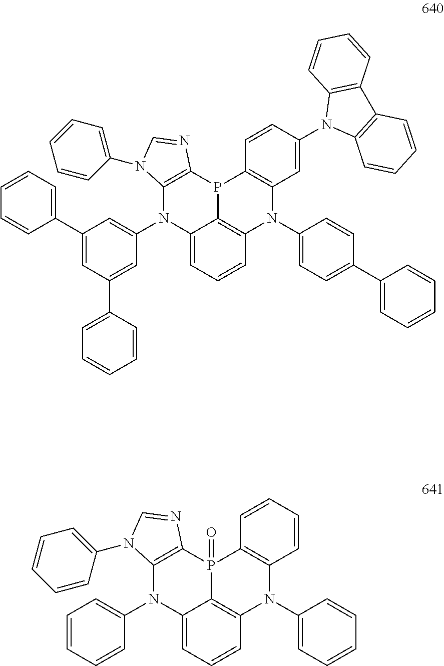 [SMILES: c1ccc(-c2ccc(N3c4cc(-n5c6ccccc6c6ccccc65)ccc4P4c5ncn(-c6ccccc6)c5N(c5cc(-c6ccccc6)cc(-c6ccccc6)c5)c5cccc3c54)cc2)cc1 ; O=P12c3ccccc3N(c3ccccc3)c3cccc(c31)N(c1ccccc1)c1c2ncn1-c1ccccc1]

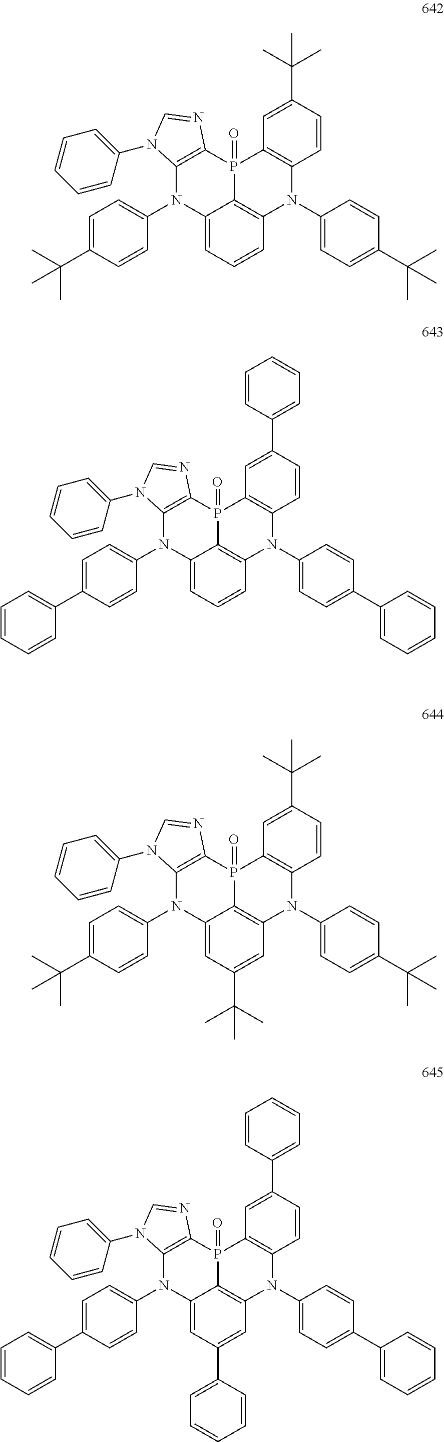

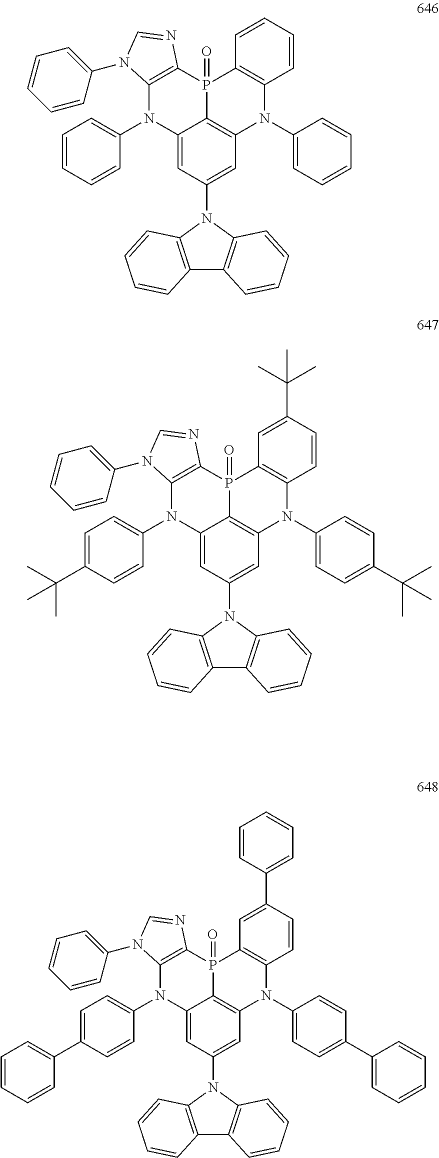 [SMILES: O=P12c3ccccc3N(c3ccccc3)c3cc(-n4c5ccccc5c5ccccc54)cc(c31)N(c1ccccc1)c1c2ncn1-c1ccccc1 ; CC(C)(C)c1ccc(N2c3ccc(C(C)(C)C)cc3P3(=O)c4ncn(-c5ccccc5)c4N(c4ccc(C(C)(C)C)cc4)c4cc(-n5c6ccccc6c6ccccc65)cc2c43)cc1 ; O=P12c3cc(-c4ccccc4)ccc3N(c3ccc(-c4ccccc4)cc3)c3cc(-n4c5ccccc5c5ccccc54)cc(c31)N(c1ccc(-c3ccccc3)cc1)c1c2ncn1-c1ccccc1]

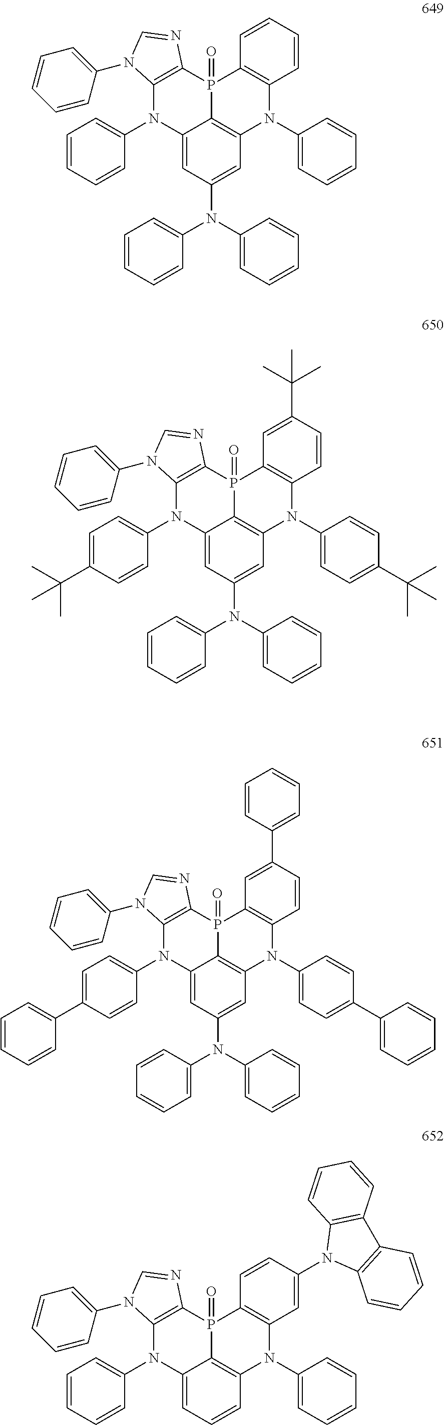 [SMILES: O=P12c3ccccc3N(c3ccccc3)c3cc(N(c4ccccc4)c4ccccc4)cc(c31)N(c1ccccc1)c1c2ncn1-c1ccccc1 ; CC(C)(C)c1ccc(N2c3ccc(C(C)(C)C)cc3P3(=O)c4ncn(-c5ccccc5)c4N(c4ccc(C(C)(C)C)cc4)c4cc(N(c5ccccc5)c5ccccc5)cc2c43)cc1 ; O=P12c3cc(-c4ccccc4)ccc3N(c3ccc(-c4ccccc4)cc3)c3cc(N(c4ccccc4)c4ccccc4)cc(c31)N(c1ccc(-c3ccccc3)cc1)c1c2ncn1-c1ccccc1 ; O=P12c3ccc(-n4c5ccccc5c5ccccc54)cc3N(c3ccccc3)c3cccc(c31)N(c1ccccc1)c1c2ncn1-c1ccccc1]

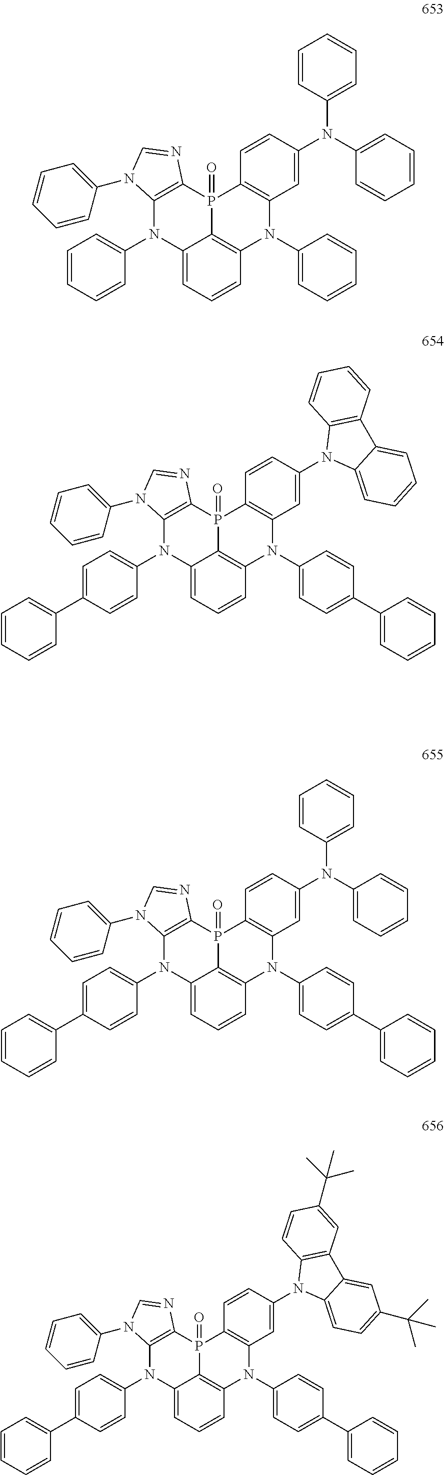 [SMILES: O=P12c3ccc(N(c4ccccc4)c4ccccc4)cc3N(c3ccccc3)c3cccc(c31)N(c1ccccc1)c1c2ncn1-c1ccccc1 ; O=P12c3ccc(-n4c5ccccc5c5ccccc54)cc3N(c3ccc(-c4ccccc4)cc3)c3cccc(c31)N(c1ccc(-c3ccccc3)cc1)c1c2ncn1-c1ccccc1 ; O=P12c3ccc(N(c4ccccc4)c4ccccc4)cc3N(c3ccc(-c4ccccc4)cc3)c3cccc(c31)N(c1ccc(-c3ccccc3)cc1)c1c2ncn1-c1ccccc1 ; CC(C)(C)c1ccc2c(c1)c1cc(C(C)(C)C)ccc1n2-c1ccc2c(c1)N(c1ccc(-c3ccccc3)cc1)c1cccc3c1P2(=O)c1ncn(-c2ccccc2)c1N3c1ccc(-c2ccccc2)cc1]

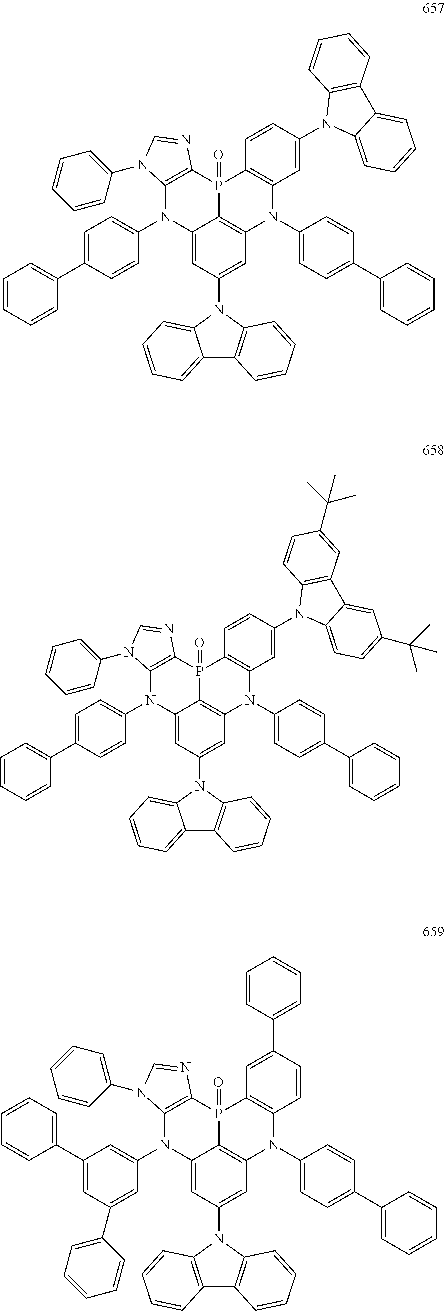 [SMILES: O=P12c3ccc(-n4c5ccccc5c5ccccc54)cc3N(c3ccc(-c4ccccc4)cc3)c3cc(-n4c5ccccc5c5ccccc54)cc(c31)N(c1ccc(-c3ccccc3)cc1)c1c2ncn1-c1ccccc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2(=O)c1ncn(-c2ccccc2)c1N3c1ccc(-c2ccccc2)cc1 ; O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1c2ncn1-c1ccccc1]

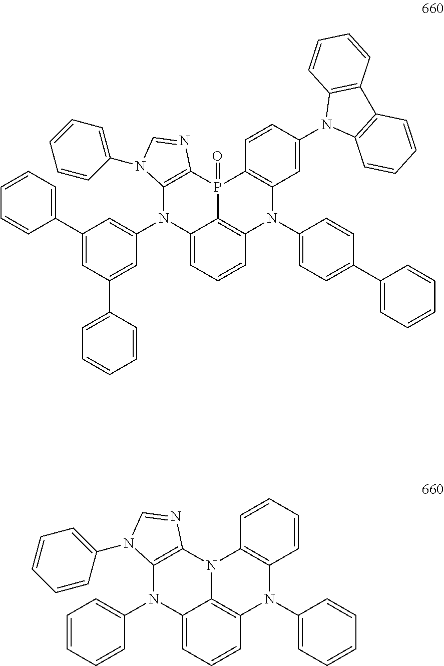 [SMILES: O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1c2ncn1-c1ccccc1 ; c1ccc(N2c3ccccc3N3c4ncn(-c5ccccc5)c4N(c4ccccc4)c4cccc2c43)cc1]

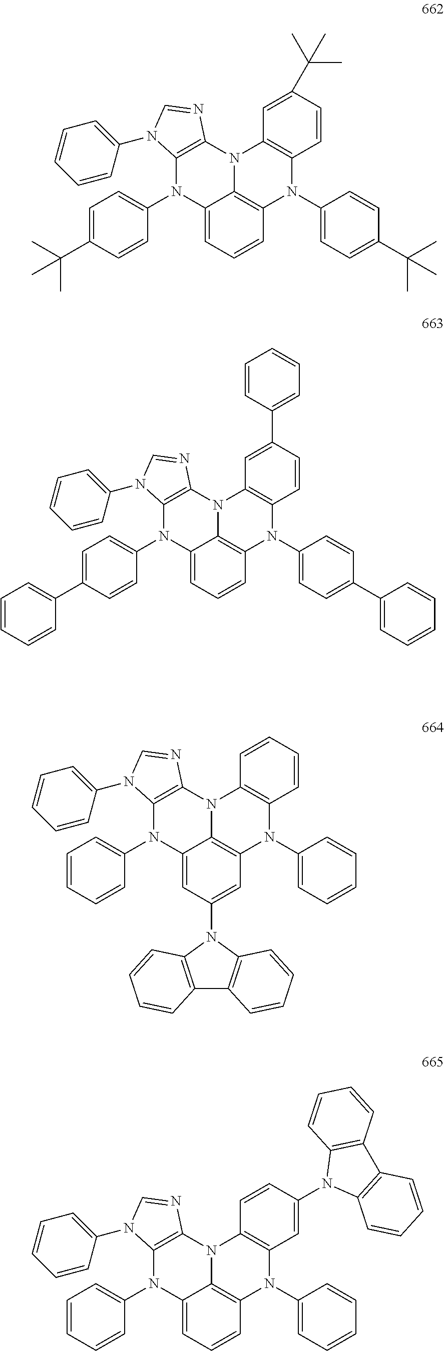 [SMILES: CC(C)(C)c1ccc(N2c3ccc(C(C)(C)C)cc3N3c4ncn(-c5ccccc5)c4N(c4ccc(C(C)(C)C)cc4)c4cccc2c43)cc1 ; c1ccc(-c2ccc(N3c4ccc(-c5ccccc5)cc4N4c5ncn(-c6ccccc6)c5N(c5ccc(-c6ccccc6)cc5)c5cccc3c54)cc2)cc1 ; c1ccc(N2c3ccccc3N3c4ncn(-c5ccccc5)c4N(c4ccccc4)c4cc(-n5c6ccccc6c6ccccc65)cc2c43)cc1 ; c1ccc(N2c3cc(-n4c5ccccc5c5ccccc54)ccc3N3c4ncn(-c5ccccc5)c4N(c4ccccc4)c4cccc2c43)cc1]

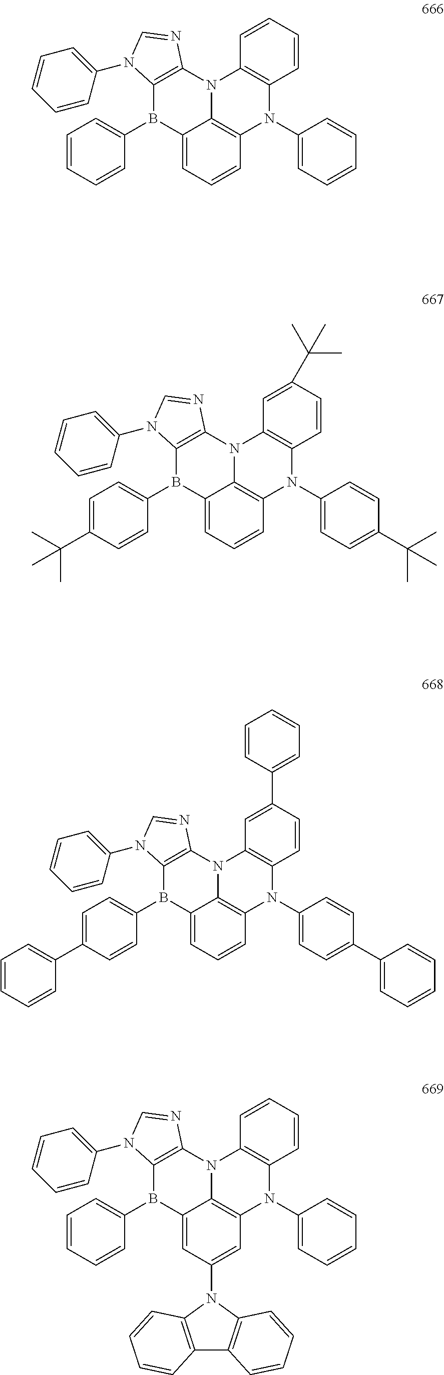 [SMILES: c1ccc(B2c3cccc4c3N(c3ccccc3N4c3ccccc3)c3ncn(-c4ccccc4)c32)cc1 ; CC(C)(C)c1ccc(B2c3cccc4c3N(c3cc(C(C)(C)C)ccc3N4c3ccc(C(C)(C)C)cc3)c3ncn(-c4ccccc4)c32)cc1 ; c1ccc(-c2ccc(B3c4cccc5c4N(c4cc(-c6ccccc6)ccc4N5c4ccc(-c5ccccc5)cc4)c4ncn(-c5ccccc5)c43)cc2)cc1 ; c1ccc(B2c3cc(-n4c5ccccc5c5ccccc54)cc4c3N(c3ccccc3N4c3ccccc3)c3ncn(-c4ccccc4)c32)cc1]

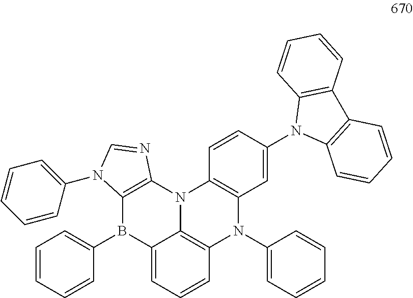 [SMILES: c1ccc(B2c3cccc4c3N(c3ccc(-n5c6ccccc6c6ccccc65)cc3N4c3ccccc3)c3ncn(-c4ccccc4)c32)cc1]

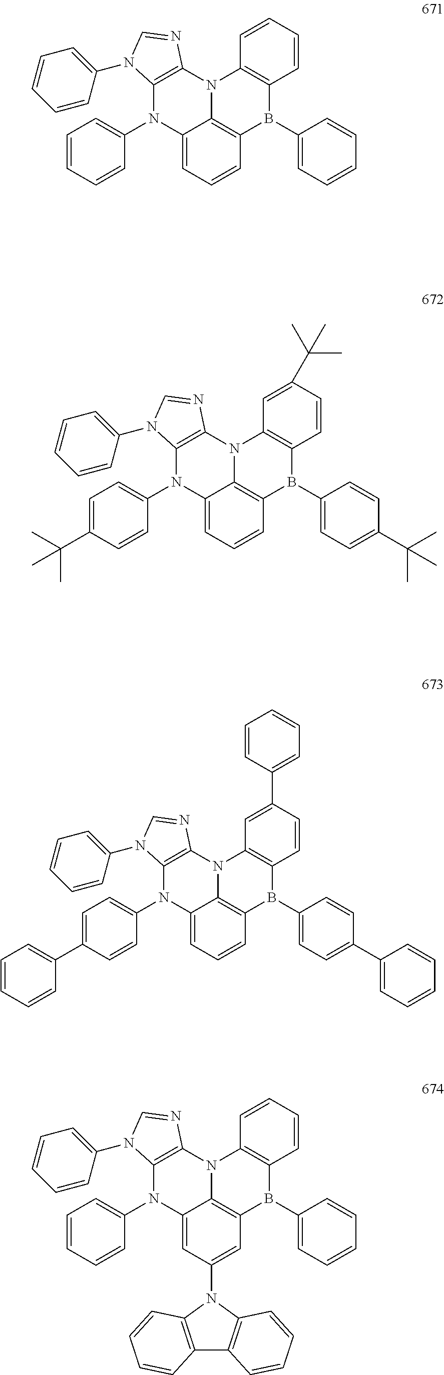 [SMILES: c1ccc(B2c3ccccc3N3c4ncn(-c5ccccc5)c4N(c4ccccc4)c4cccc2c43)cc1 ; CC(C)(C)c1ccc(B2c3ccc(C(C)(C)C)cc3N3c4ncn(-c5ccccc5)c4N(c4ccc(C(C)(C)C)cc4)c4cccc2c43)cc1 ; c1ccc(-c2ccc(B3c4ccc(-c5ccccc5)cc4N4c5ncn(-c6ccccc6)c5N(c5ccc(-c6ccccc6)cc5)c5cccc3c54)cc2)cc1 ; c1ccc(B2c3ccccc3N3c4ncn(-c5ccccc5)c4N(c4ccccc4)c4cc(-n5c6ccccc6c6ccccc65)cc2c43)cc1]

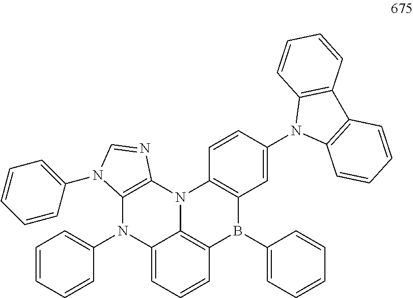 [SMILES: c1ccc(B2c3cc(-n4c5ccccc5c5ccccc54)ccc3N3c4ncn(-c5ccccc5)c4N(c4ccccc4)c4cccc2c43)cc1]

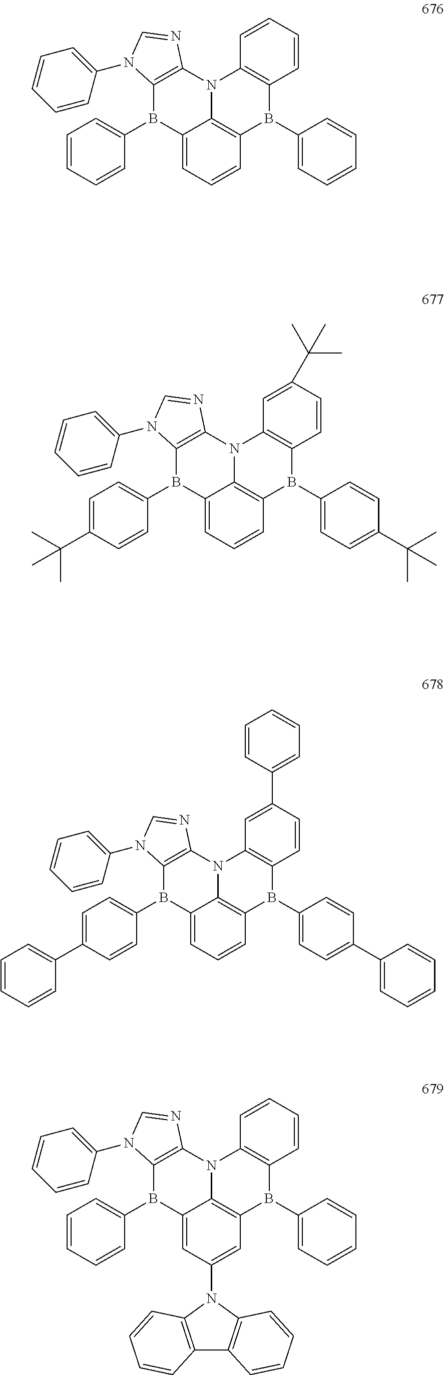 [SMILES: c1ccc(B2c3ccccc3N3c4ncn(-c5ccccc5)c4B(c4ccccc4)c4cccc2c43)cc1 ; CC(C)(C)c1ccc(B2c3ccc(C(C)(C)C)cc3N3c4ncn(-c5ccccc5)c4B(c4ccc(C(C)(C)C)cc4)c4cccc2c43)cc1 ; c1ccc(-c2ccc(B3c4ccc(-c5ccccc5)cc4N4c5ncn(-c6ccccc6)c5B(c5ccc(-c6ccccc6)cc5)c5cccc3c54)cc2)cc1 ; c1ccc(B2c3ccccc3N3c4ncn(-c5ccccc5)c4B(c4ccccc4)c4cc(-n5c6ccccc6c6ccccc65)cc2c43)cc1]

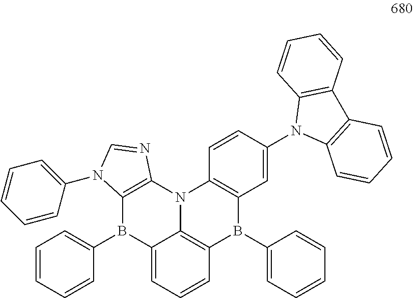 [SMILES: c1ccc(B2c3cc(-n4c5ccccc5c5ccccc54)ccc3N3c4ncn(-c5ccccc5)c4B(c4ccccc4)c4cccc2c43)cc1]

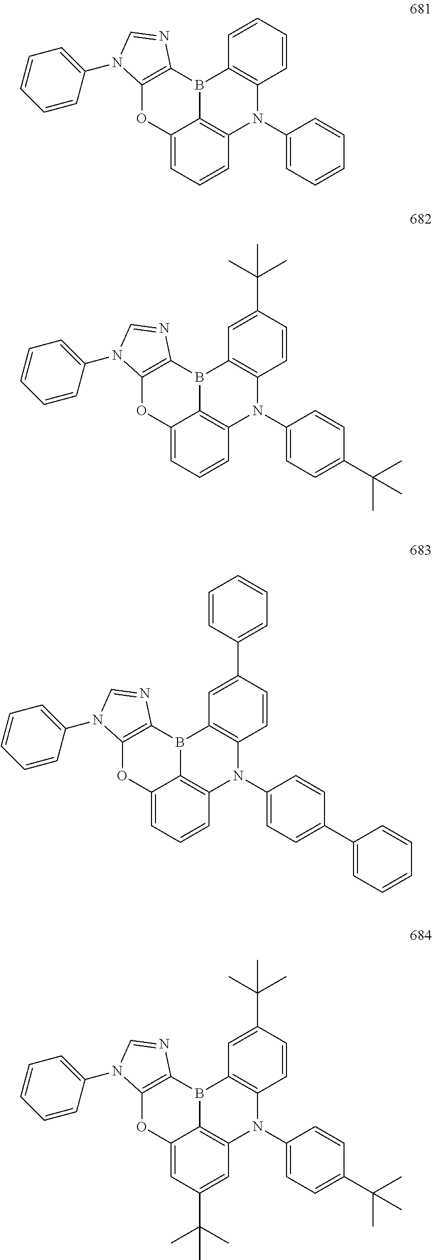 [SMILES: c1ccc(N2c3ccccc3B3c4ncn(-c5ccccc5)c4Oc4cccc2c43)cc1 ; CC(C)(C)c1ccc(N2c3ccc(C(C)(C)C)cc3B3c4ncn(-c5ccccc5)c4Oc4cccc2c43)cc1 ; c1ccc(-c2ccc(N3c4ccc(-c5ccccc5)cc4B4c5ncn(-c6ccccc6)c5Oc5cccc3c54)cc2)cc1 ; CC(C)(C)c1ccc(N2c3ccc(C(C)(C)C)cc3B3c4ncn(-c5ccccc5)c4Oc4cc(C(C)(C)C)cc2c43)cc1]

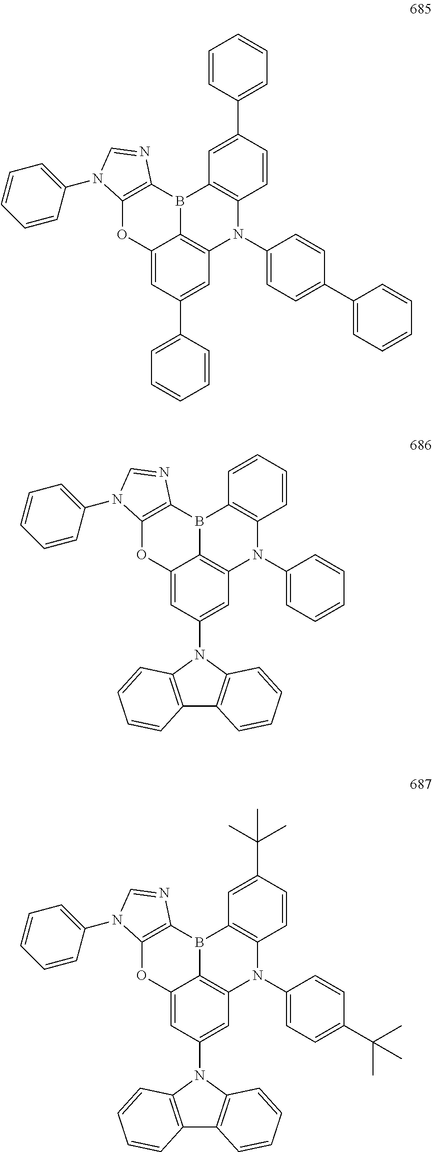 [SMILES: c1ccc(-c2ccc(N3c4ccc(-c5ccccc5)cc4B4c5ncn(-c6ccccc6)c5Oc5cc(-c6ccccc6)cc3c54)cc2)cc1 ; c1ccc(N2c3ccccc3B3c4ncn(-c5ccccc5)c4Oc4cc(-n5c6ccccc6c6ccccc65)cc2c43)cc1 ; CC(C)(C)c1ccc(N2c3ccc(C(C)(C)C)cc3B3c4ncn(-c5ccccc5)c4Oc4cc(-n5c6ccccc6c6ccccc65)cc2c43)cc1]

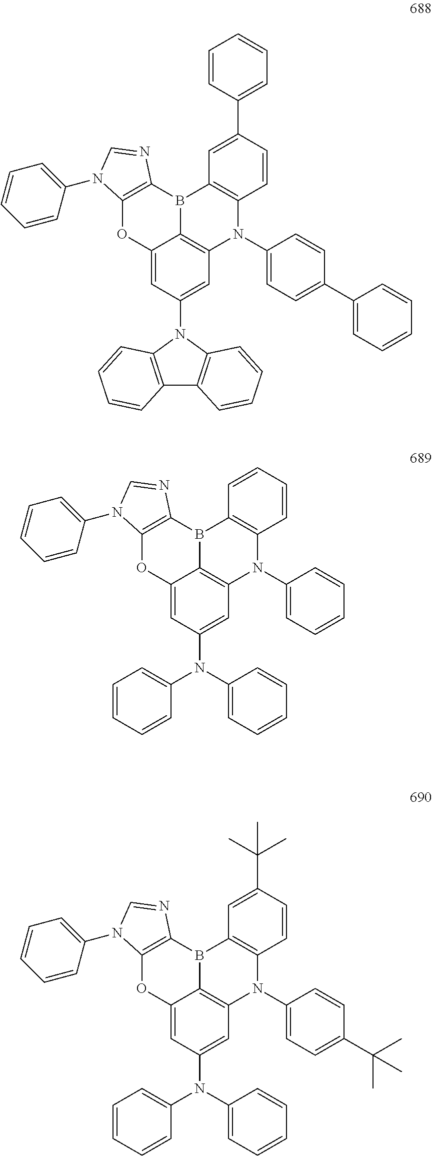 [SMILES: c1ccc(-c2ccc(N3c4ccc(-c5ccccc5)cc4B4c5ncn(-c6ccccc6)c5Oc5cc(-n6c7ccccc7c7ccccc76)cc3c54)cc2)cc1 ; c1ccc(N(c2ccccc2)c2cc3c4c(c2)N(c2ccccc2)c2ccccc2B4c2ncn(-c4ccccc4)c2O3)cc1 ; CC(C)(C)c1ccc(N2c3ccc(C(C)(C)C)cc3B3c4ncn(-c5ccccc5)c4Oc4cc(N(c5ccccc5)c5ccccc5)cc2c43)cc1]

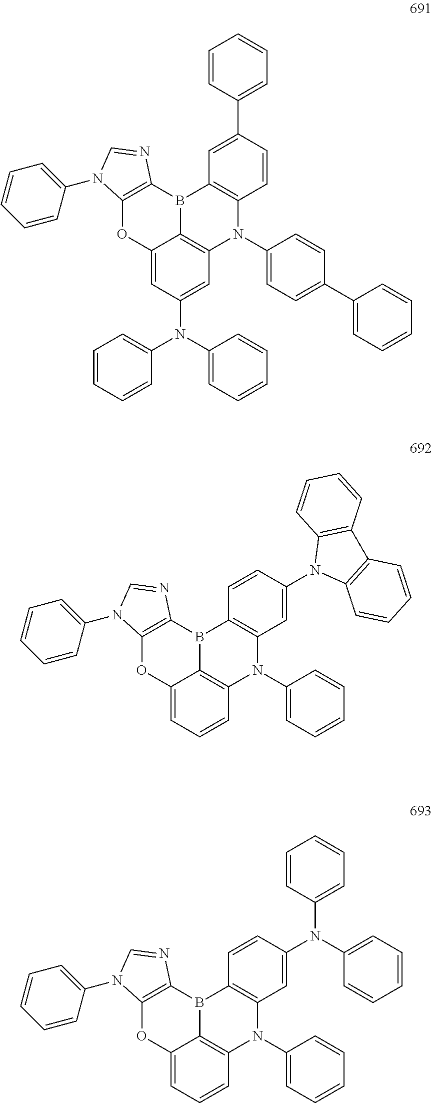 [SMILES: c1ccc(-c2ccc(N3c4ccc(-c5ccccc5)cc4B4c5ncn(-c6ccccc6)c5Oc5cc(N(c6ccccc6)c6ccccc6)cc3c54)cc2)cc1 ; c1ccc(N2c3cc(-n4c5ccccc5c5ccccc54)ccc3B3c4ncn(-c5ccccc5)c4Oc4cccc2c43)cc1 ; c1ccc(N(c2ccccc2)c2ccc3c(c2)N(c2ccccc2)c2cccc4c2B3c2ncn(-c3ccccc3)c2O4)cc1]

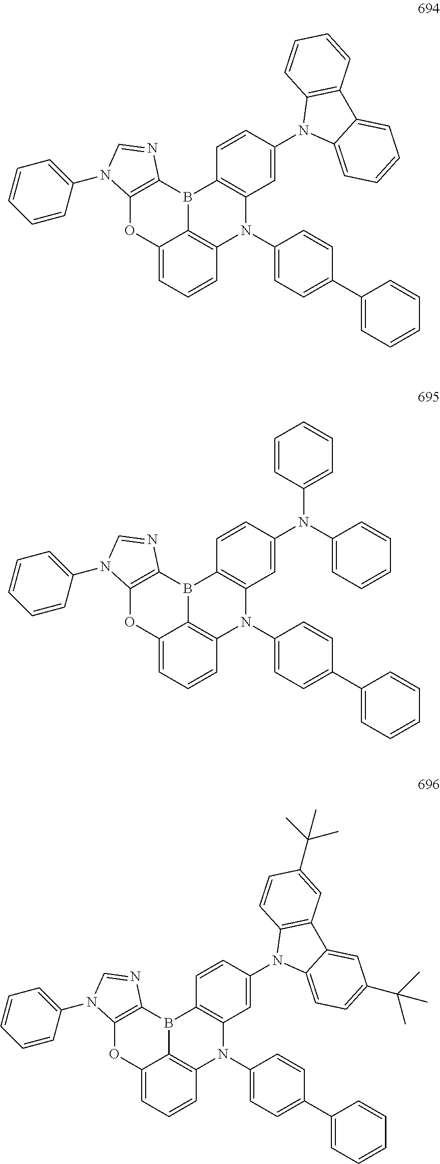 [SMILES: c1ccc(-c2ccc(N3c4cc(-n5c6ccccc6c6ccccc65)ccc4B4c5ncn(-c6ccccc6)c5Oc5cccc3c54)cc2)cc1 ; c1ccc(-c2ccc(N3c4cc(N(c5ccccc5)c5ccccc5)ccc4B4c5ncn(-c6ccccc6)c5Oc5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ncn(-c2ccccc2)c1O3]

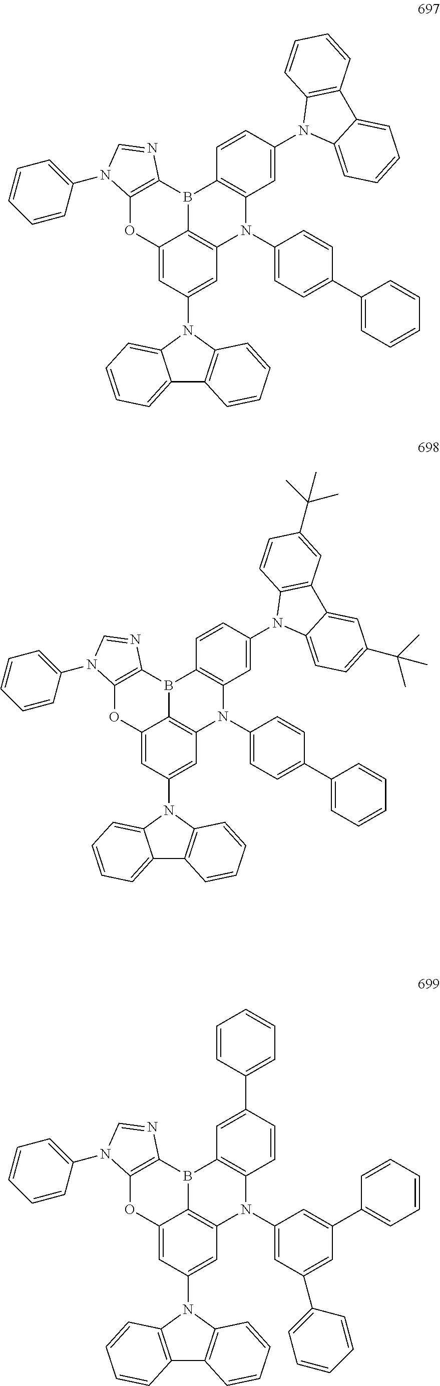 [SMILES: c1ccc(-c2ccc(N3c4cc(-n5c6ccccc6c6ccccc65)ccc4B4c5ncn(-c6ccccc6)c5Oc5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ncn(-c2ccccc2)c1O3 ; c1ccc(-c2cc(-c3ccccc3)cc(N3c4ccc(-c5ccccc5)cc4B4c5ncn(-c6ccccc6)c5Oc5cc(-n6c7ccccc7c7ccccc76)cc3c54)c2)cc1]

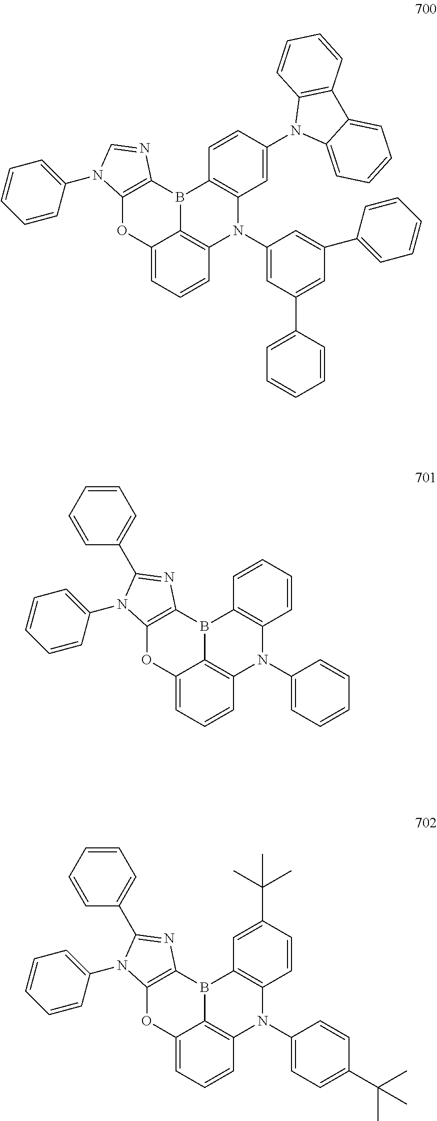 [SMILES: c1ccc(-c2cc(-c3ccccc3)cc(N3c4cc(-n5c6ccccc6c6ccccc65)ccc4B4c5ncn(-c6ccccc6)c5Oc5cccc3c54)c2)cc1 ; c1ccc(-c2nc3c(n2-c2ccccc2)Oc2cccc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4Oc4cccc2c43)cc1]

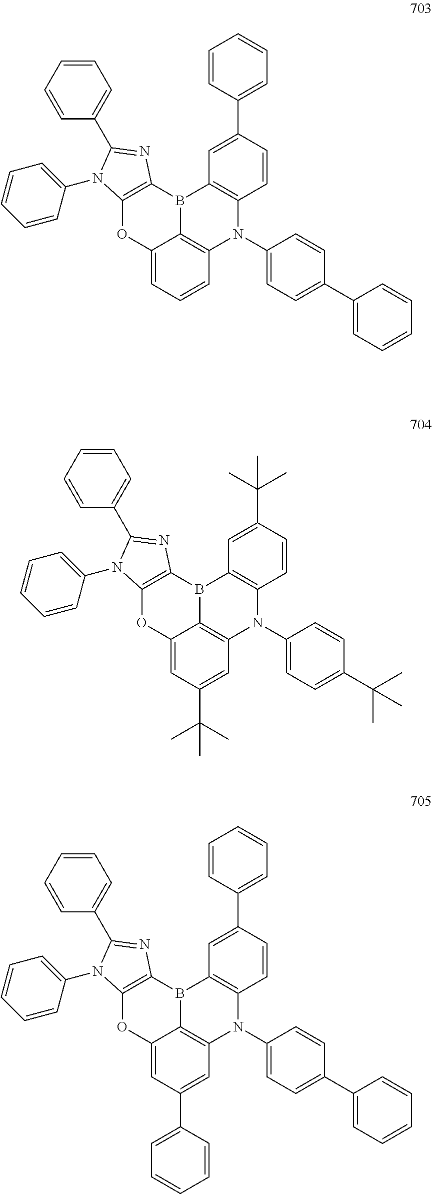 [SMILES: c1ccc(-c2ccc(N3c4ccc(-c5ccccc5)cc4B4c5nc(-c6ccccc6)n(-c6ccccc6)c5Oc5cccc3c54)cc2)cc1 ; CC(C)(C)c1ccc(N2c3ccc(C(C)(C)C)cc3B3c4nc(-c5ccccc5)n(-c5ccccc5)c4Oc4cc(C(C)(C)C)cc2c43)cc1 ; c1ccc(-c2ccc(N3c4ccc(-c5ccccc5)cc4B4c5nc(-c6ccccc6)n(-c6ccccc6)c5Oc5cc(-c6ccccc6)cc3c54)cc2)cc1]

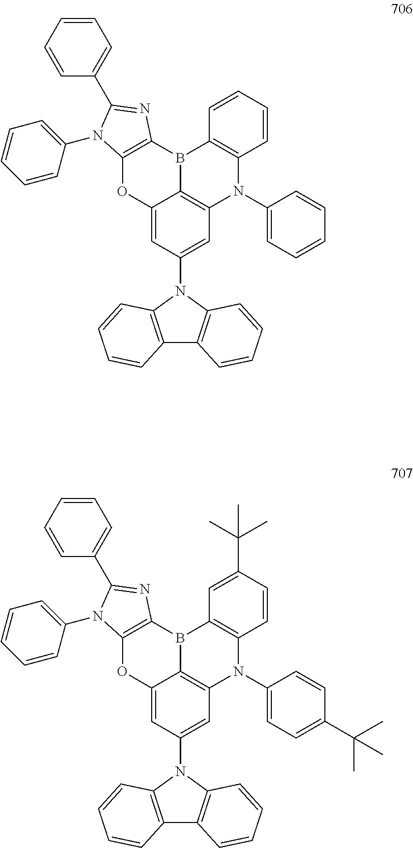 [SMILES: c1ccc(-c2nc3c(n2-c2ccccc2)Oc2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4Oc4cc(-n5c6ccccc6c6ccccc65)cc2c43)cc1]

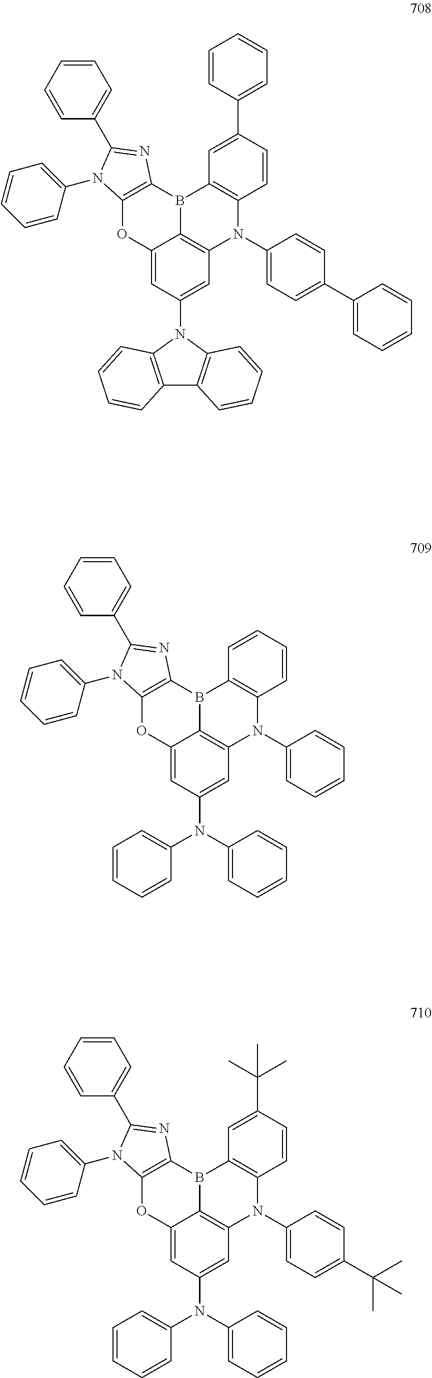 [SMILES: c1ccc(-c2ccc(N3c4ccc(-c5ccccc5)cc4B4c5nc(-c6ccccc6)n(-c6ccccc6)c5Oc5cc(-n6c7ccccc7c7ccccc76)cc3c54)cc2)cc1 ; c1ccc(-c2nc3c(n2-c2ccccc2)Oc2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4nc(-c5ccccc5)n(-c5ccccc5)c4Oc4cc(N(c5ccccc5)c5ccccc5)cc2c43)cc1]

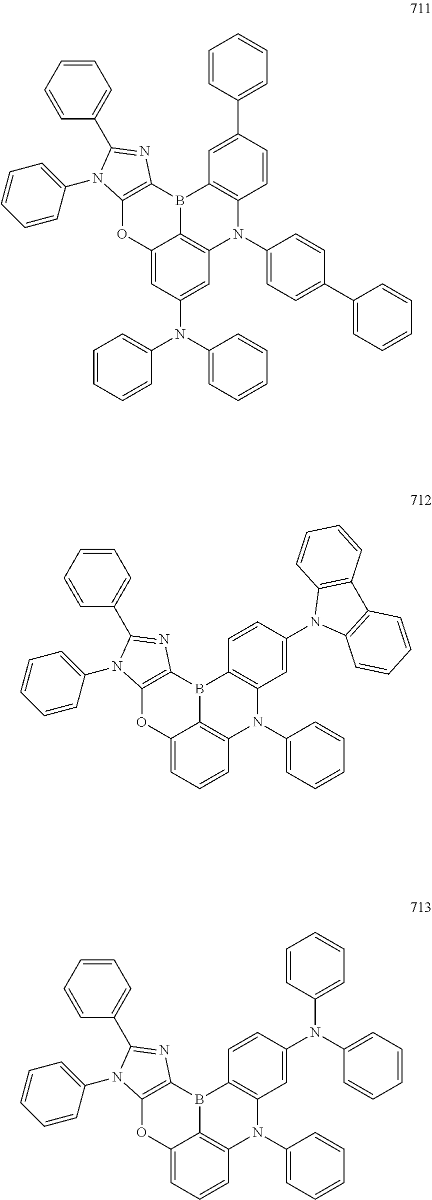 [SMILES: c1ccc(-c2ccc(N3c4ccc(-c5ccccc5)cc4B4c5nc(-c6ccccc6)n(-c6ccccc6)c5Oc5cc(N(c6ccccc6)c6ccccc6)cc3c54)cc2)cc1 ; c1ccc(-c2nc3c(n2-c2ccccc2)Oc2cccc4c2B3c2ccc(-n3c5ccccc5c5ccccc53)cc2N4c2ccccc2)cc1 ; c1ccc(-c2nc3c(n2-c2ccccc2)Oc2cccc4c2B3c2ccc(N(c3ccccc3)c3ccccc3)cc2N4c2ccccc2)cc1]

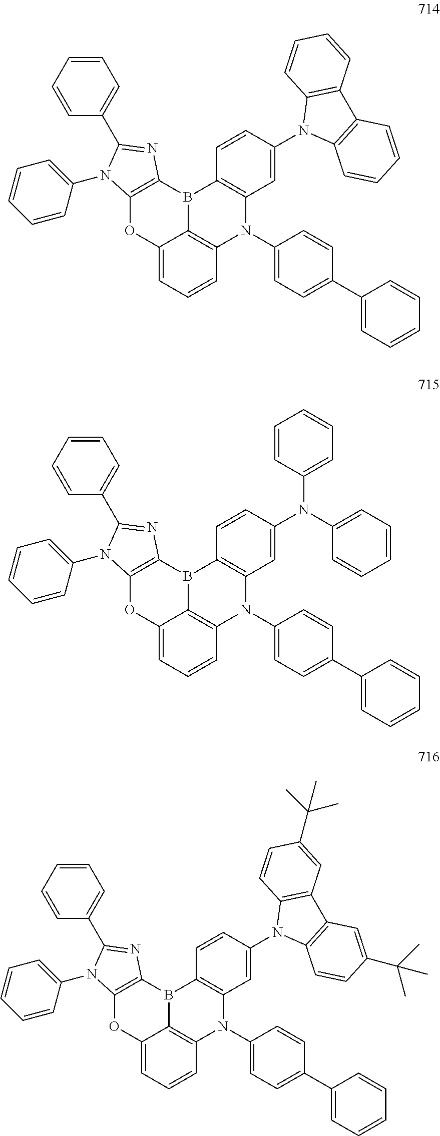 [SMILES: c1ccc(-c2ccc(N3c4cc(-n5c6ccccc6c6ccccc65)ccc4B4c5nc(-c6ccccc6)n(-c6ccccc6)c5Oc5cccc3c54)cc2)cc1 ; c1ccc(-c2ccc(N3c4cc(N(c5ccccc5)c5ccccc5)ccc4B4c5nc(-c6ccccc6)n(-c6ccccc6)c5Oc5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nc(-c2ccccc2)n(-c2ccccc2)c1O3]

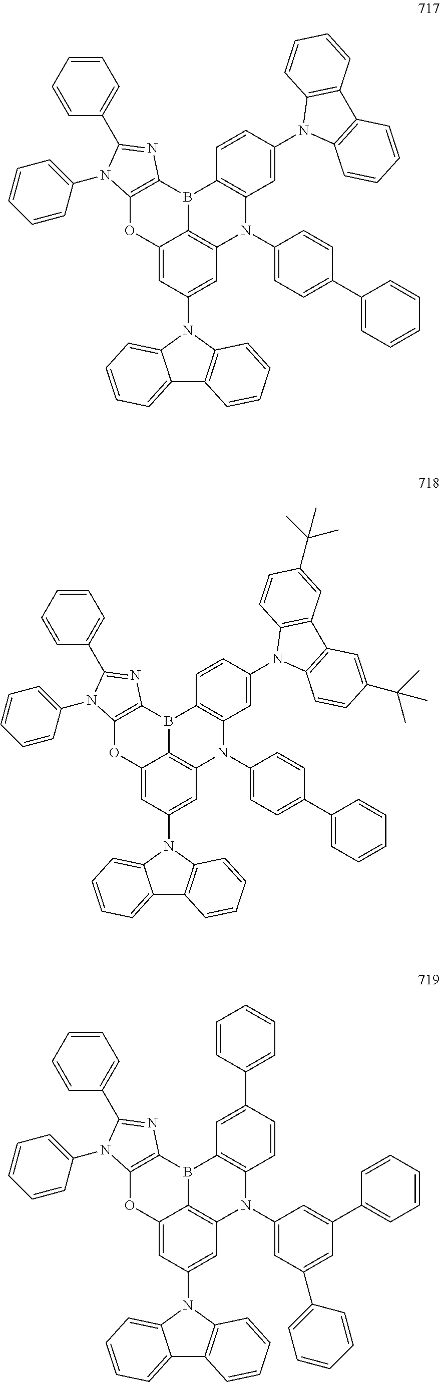 [SMILES: c1ccc(-c2ccc(N3c4cc(-n5c6ccccc6c6ccccc65)ccc4B4c5nc(-c6ccccc6)n(-c6ccccc6)c5Oc5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nc(-c2ccccc2)n(-c2ccccc2)c1O3 ; c1ccc(-c2cc(-c3ccccc3)cc(N3c4ccc(-c5ccccc5)cc4B4c5nc(-c6ccccc6)n(-c6ccccc6)c5Oc5cc(-n6c7ccccc7c7ccccc76)cc3c54)c2)cc1]

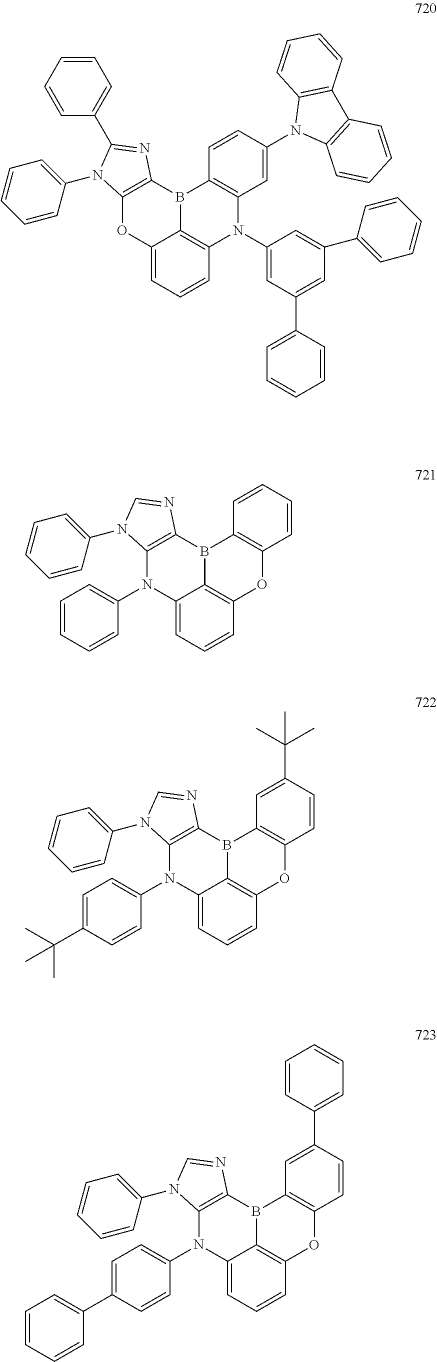 [SMILES: c1ccc(-c2cc(-c3ccccc3)cc(N3c4cc(-n5c6ccccc6c6ccccc65)ccc4B4c5nc(-c6ccccc6)n(-c6ccccc6)c5Oc5cccc3c54)c2)cc1 ; c1ccc(N2c3cccc4c3B(c3ccccc3O4)c3ncn(-c4ccccc4)c32)cc1 ; CC(C)(C)c1ccc(N2c3cccc4c3B(c3cc(C(C)(C)C)ccc3O4)c3ncn(-c4ccccc4)c32)cc1 ; c1ccc(-c2ccc(N3c4cccc5c4B(c4cc(-c6ccccc6)ccc4O5)c4ncn(-c5ccccc5)c43)cc2)cc1]

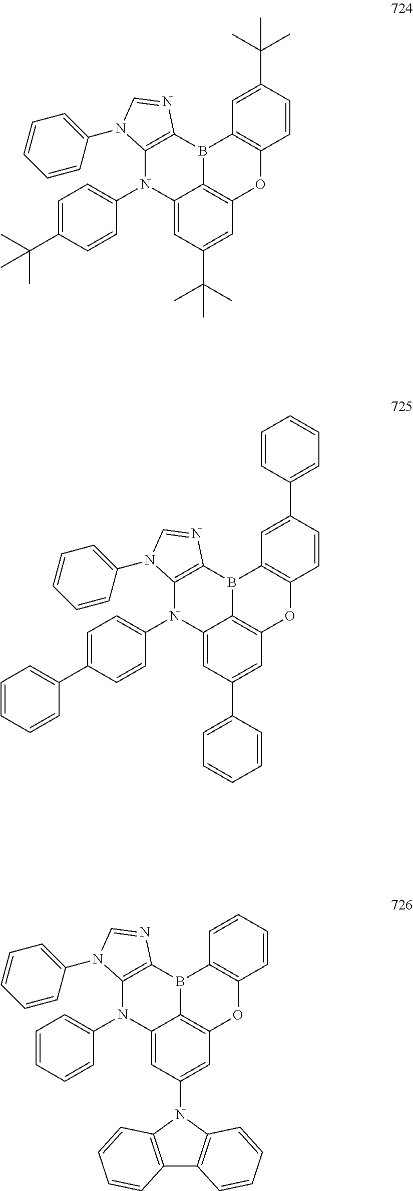 [SMILES: CC(C)(C)c1ccc(N2c3cc(C(C)(C)C)cc4c3B(c3cc(C(C)(C)C)ccc3O4)c3ncn(-c4ccccc4)c32)cc1 ; c1ccc(-c2ccc(N3c4cc(-c5ccccc5)cc5c4B(c4cc(-c6ccccc6)ccc4O5)c4ncn(-c5ccccc5)c43)cc2)cc1 ; c1ccc(N2c3cc(-n4c5ccccc5c5ccccc54)cc4c3B(c3ccccc3O4)c3ncn(-c4ccccc4)c32)cc1]

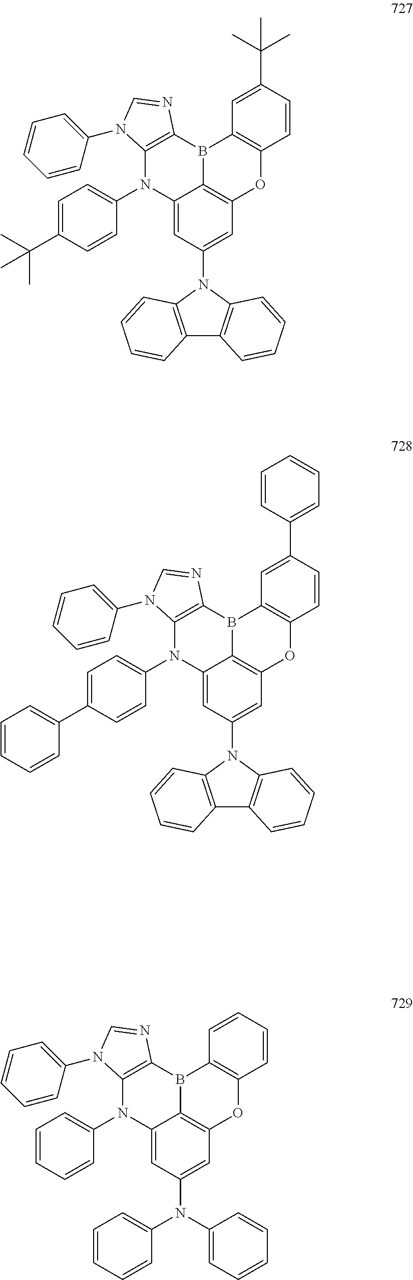 [SMILES: CC(C)(C)c1ccc(N2c3cc(-n4c5ccccc5c5ccccc54)cc4c3B(c3cc(C(C)(C)C)ccc3O4)c3ncn(-c4ccccc4)c32)cc1 ; c1ccc(-c2ccc(N3c4cc(-n5c6ccccc6c6ccccc65)cc5c4B(c4cc(-c6ccccc6)ccc4O5)c4ncn(-c5ccccc5)c43)cc2)cc1 ; c1ccc(N(c2ccccc2)c2cc3c4c(c2)N(c2ccccc2)c2c(ncn2-c2ccccc2)B4c2ccccc2O3)cc1]

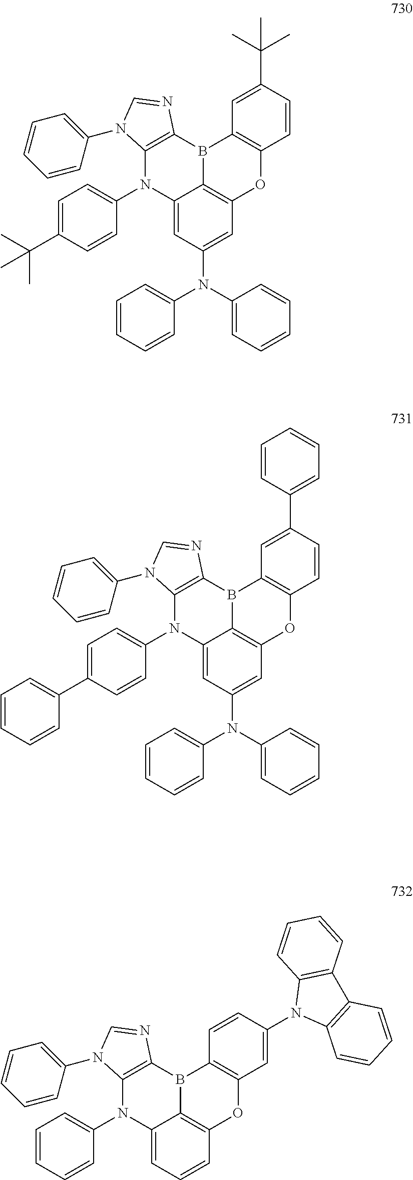 [SMILES: CC(C)(C)c1ccc(N2c3cc(N(c4ccccc4)c4ccccc4)cc4c3B(c3cc(C(C)(C)C)ccc3O4)c3ncn(-c4ccccc4)c32)cc1 ; c1ccc(-c2ccc(N3c4cc(N(c5ccccc5)c5ccccc5)cc5c4B(c4cc(-c6ccccc6)ccc4O5)c4ncn(-c5ccccc5)c43)cc2)cc1 ; c1ccc(N2c3cccc4c3B(c3ccc(-n5c6ccccc6c6ccccc65)cc3O4)c3ncn(-c4ccccc4)c32)cc1]

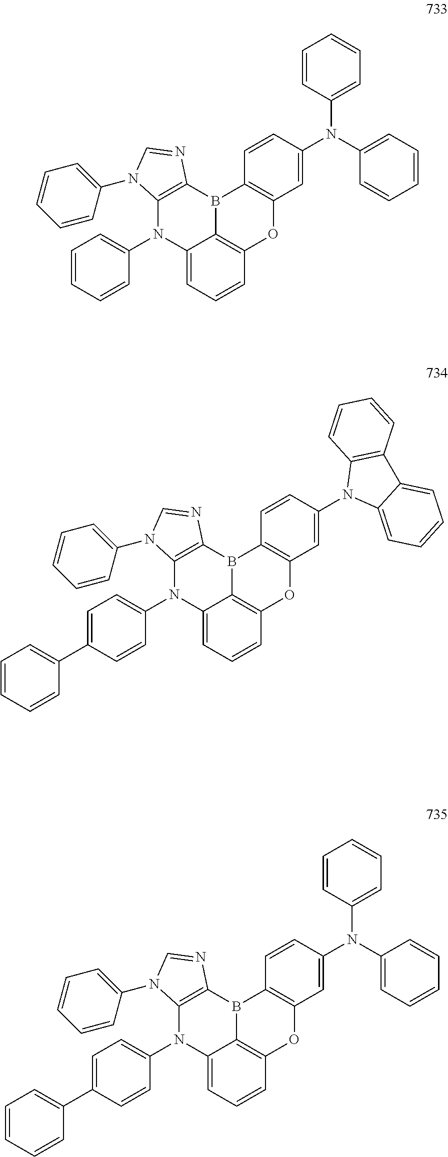 [SMILES: c1ccc(N(c2ccccc2)c2ccc3c(c2)Oc2cccc4c2B3c2ncn(-c3ccccc3)c2N4c2ccccc2)cc1 ; c1ccc(-c2ccc(N3c4cccc5c4B(c4ccc(-n6c7ccccc7c7ccccc76)cc4O5)c4ncn(-c5ccccc5)c43)cc2)cc1 ; c1ccc(-c2ccc(N3c4cccc5c4B(c4ccc(N(c6ccccc6)c6ccccc6)cc4O5)c4ncn(-c5ccccc5)c43)cc2)cc1]

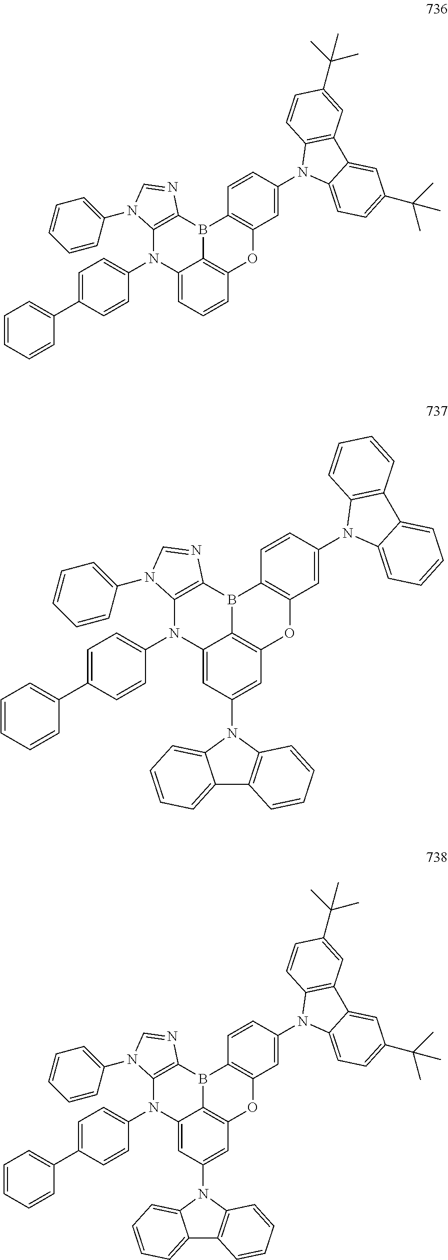 [SMILES: CC(C)(C)c1ccc2c(c1)c1cc(C(C)(C)C)ccc1n2-c1ccc2c(c1)Oc1cccc3c1B2c1ncn(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4ncn(-c5ccccc5)c43)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1ncn(-c2ccccc2)c1N3c1ccc(-c2ccccc2)cc1]

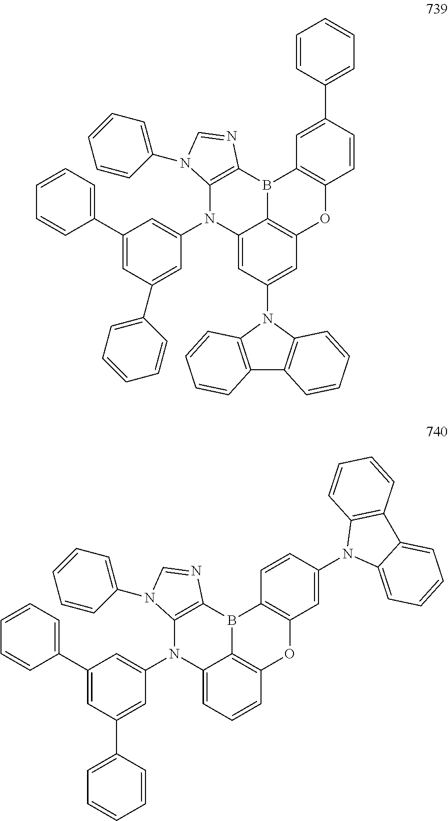 [SMILES: c1ccc(-c2cc(-c3ccccc3)cc(N3c4cc(-n5c6ccccc6c6ccccc65)cc5c4B(c4cc(-c6ccccc6)ccc4O5)c4ncn(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4ncn(-c5ccccc5)c43)c2)cc1]

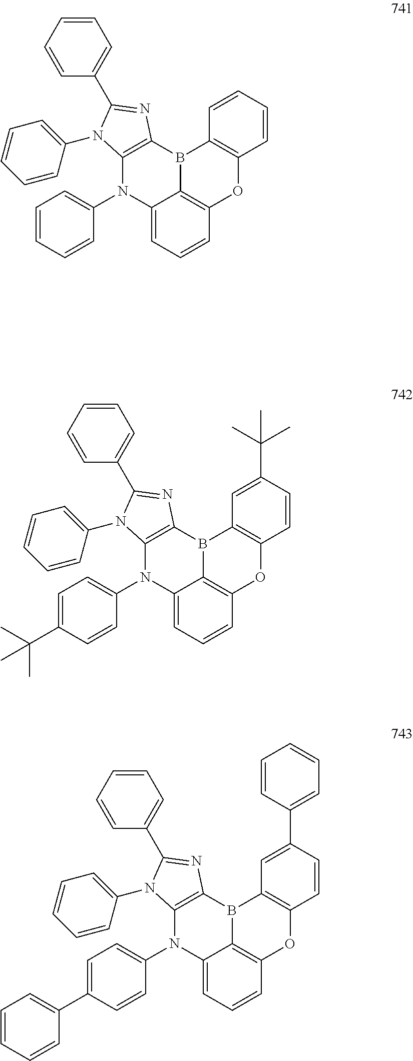 [SMILES: c1ccc(-c2nc3c(n2-c2ccccc2)N(c2ccccc2)c2cccc4c2B3c2ccccc2O4)cc1 ; CC(C)(C)c1ccc(N2c3cccc4c3B(c3cc(C(C)(C)C)ccc3O4)c3nc(-c4ccccc4)n(-c4ccccc4)c32)cc1 ; c1ccc(-c2ccc(N3c4cccc5c4B(c4cc(-c6ccccc6)ccc4O5)c4nc(-c5ccccc5)n(-c5ccccc5)c43)cc2)cc1]

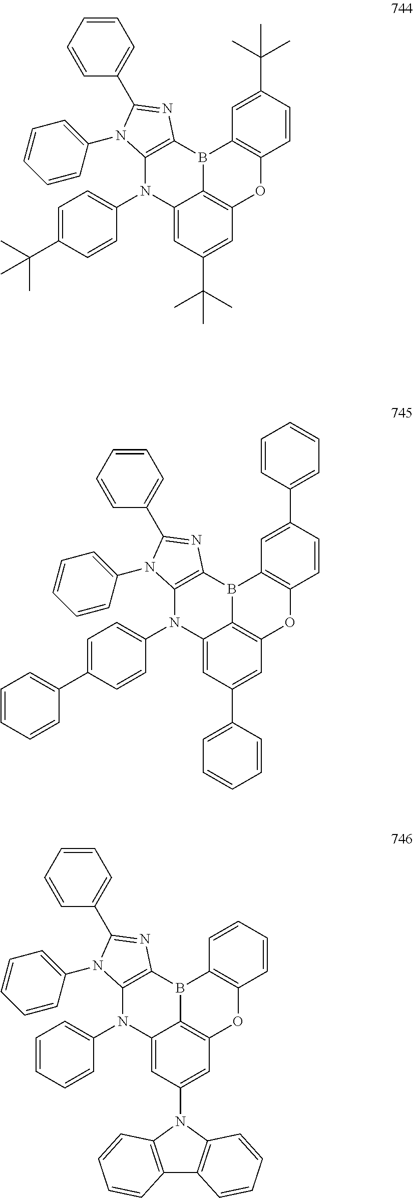 [SMILES: CC(C)(C)c1ccc(N2c3cc(C(C)(C)C)cc4c3B(c3cc(C(C)(C)C)ccc3O4)c3nc(-c4ccccc4)n(-c4ccccc4)c32)cc1 ; c1ccc(-c2ccc(N3c4cc(-c5ccccc5)cc5c4B(c4cc(-c6ccccc6)ccc4O5)c4nc(-c5ccccc5)n(-c5ccccc5)c43)cc2)cc1 ; c1ccc(-c2nc3c(n2-c2ccccc2)N(c2ccccc2)c2cc(-n4c5ccccc5c5ccccc54)cc4c2B3c2ccccc2O4)cc1]

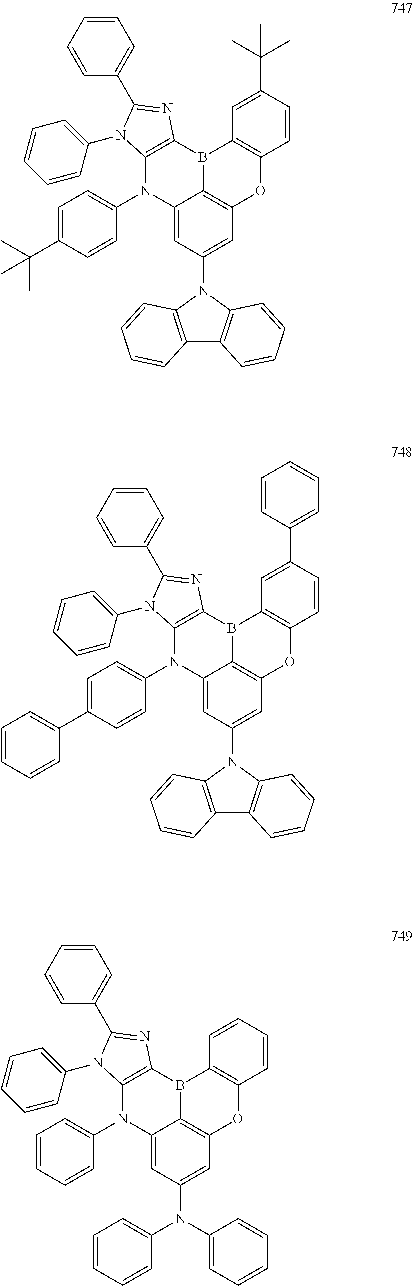 [SMILES: CC(C)(C)c1ccc(N2c3cc(-n4c5ccccc5c5ccccc54)cc4c3B(c3cc(C(C)(C)C)ccc3O4)c3nc(-c4ccccc4)n(-c4ccccc4)c32)cc1 ; c1ccc(-c2ccc(N3c4cc(-n5c6ccccc6c6ccccc65)cc5c4B(c4cc(-c6ccccc6)ccc4O5)c4nc(-c5ccccc5)n(-c5ccccc5)c43)cc2)cc1 ; c1ccc(-c2nc3c(n2-c2ccccc2)N(c2ccccc2)c2cc(N(c4ccccc4)c4ccccc4)cc4c2B3c2ccccc2O4)cc1]

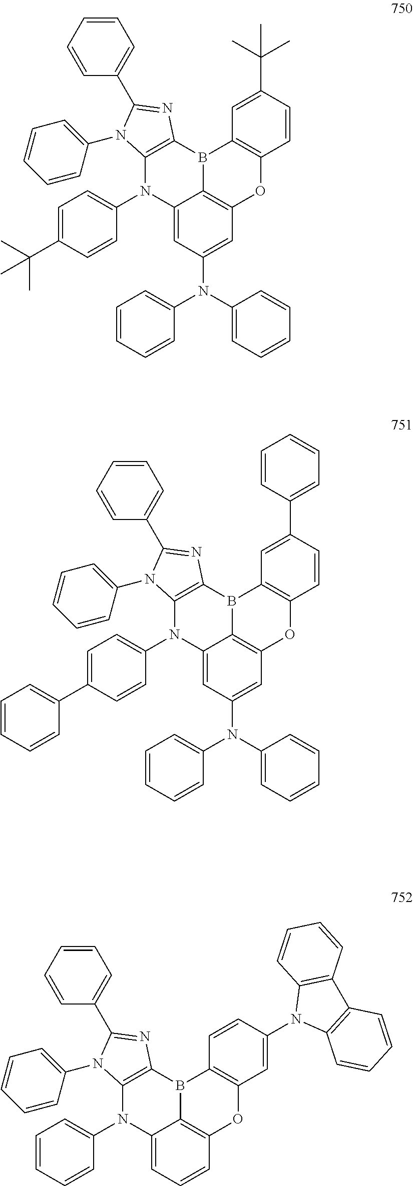 [SMILES: CC(C)(C)c1ccc(N2c3cc(N(c4ccccc4)c4ccccc4)cc4c3B(c3cc(C(C)(C)C)ccc3O4)c3nc(-c4ccccc4)n(-c4ccccc4)c32)cc1 ; c1ccc(-c2ccc(N3c4cc(N(c5ccccc5)c5ccccc5)cc5c4B(c4cc(-c6ccccc6)ccc4O5)c4nc(-c5ccccc5)n(-c5ccccc5)c43)cc2)cc1 ; c1ccc(-c2nc3c(n2-c2ccccc2)N(c2ccccc2)c2cccc4c2B3c2ccc(-n3c5ccccc5c5ccccc53)cc2O4)cc1]

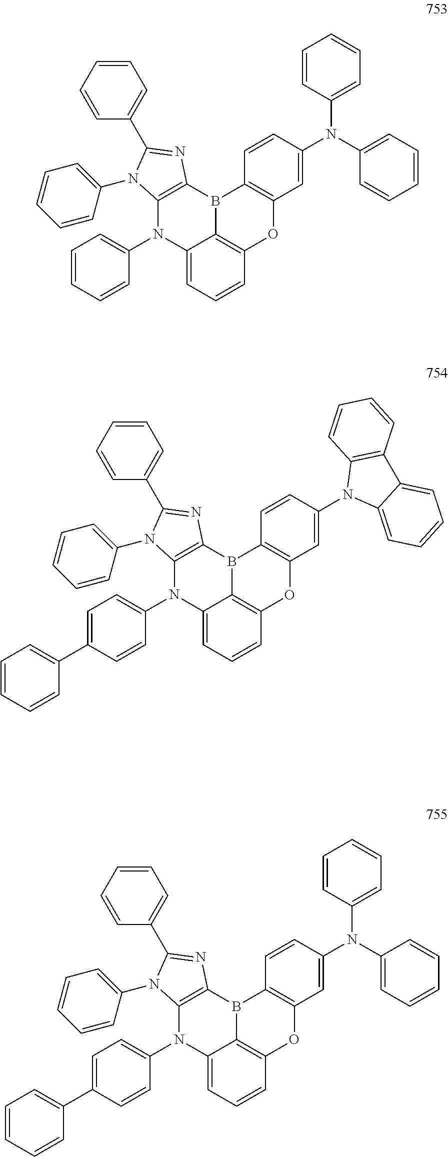 [SMILES: c1ccc(-c2nc3c(n2-c2ccccc2)N(c2ccccc2)c2cccc4c2B3c2ccc(N(c3ccccc3)c3ccccc3)cc2O4)cc1 ; c1ccc(-c2ccc(N3c4cccc5c4B(c4ccc(-n6c7ccccc7c7ccccc76)cc4O5)c4nc(-c5ccccc5)n(-c5ccccc5)c43)cc2)cc1 ; c1ccc(-c2ccc(N3c4cccc5c4B(c4ccc(N(c6ccccc6)c6ccccc6)cc4O5)c4nc(-c5ccccc5)n(-c5ccccc5)c43)cc2)cc1]

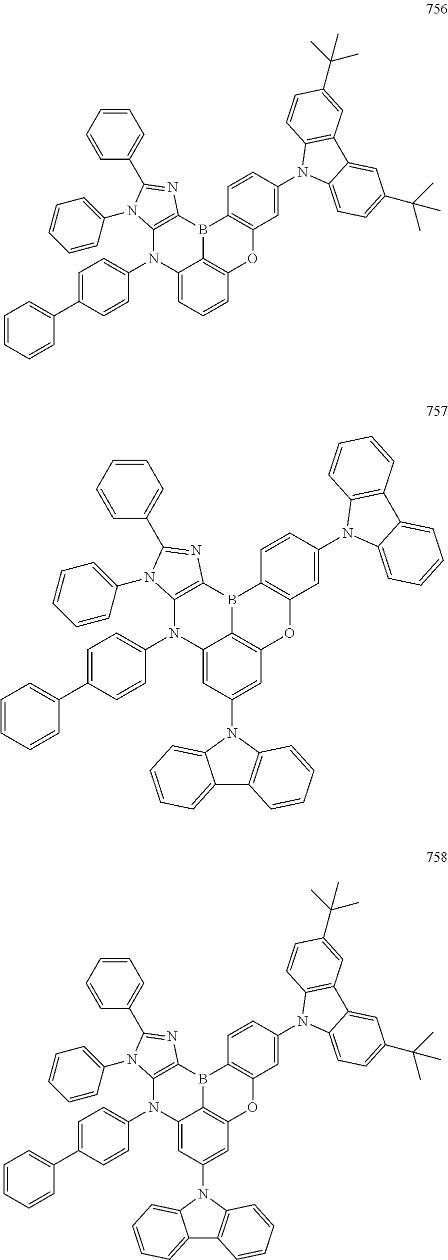 [SMILES: CC(C)(C)c1ccc2c(c1)c1cc(C(C)(C)C)ccc1n2-c1ccc2c(c1)Oc1cccc3c1B2c1nc(-c2ccccc2)n(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4nc(-c5ccccc5)n(-c5ccccc5)c43)cc2)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1nc(-c2ccccc2)n(-c2ccccc2)c1N3c1ccc(-c2ccccc2)cc1]

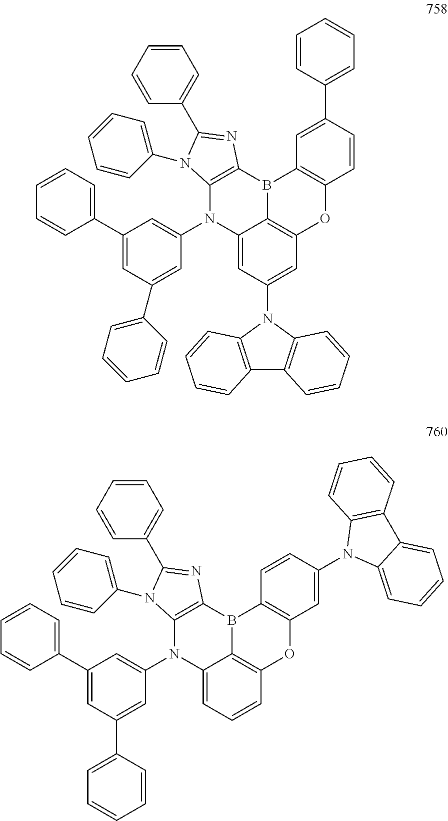 [SMILES: c1ccc(-c2cc(-c3ccccc3)cc(N3c4cc(-n5c6ccccc6c6ccccc65)cc5c4B(c4cc(-c6ccccc6)ccc4O5)c4nc(-c5ccccc5)n(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4nc(-c5ccccc5)n(-c5ccccc5)c43)c2)cc1]

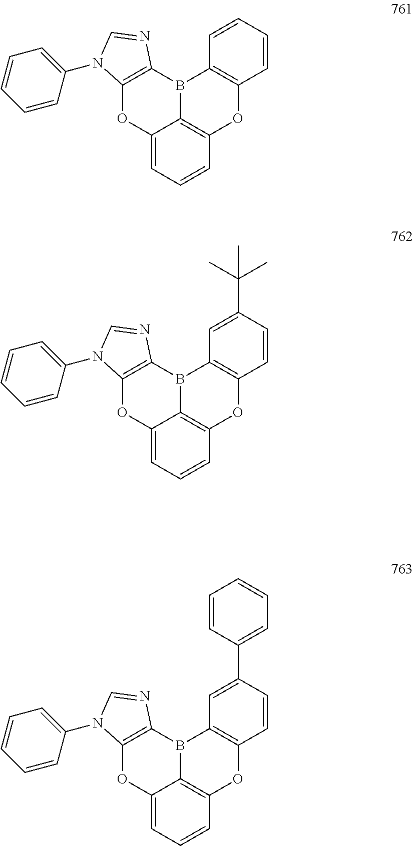 [SMILES: c1ccc(-n2cnc3c2Oc2cccc4c2B3c2ccccc2O4)cc1 ; CC(C)(C)c1ccc2c(c1)B1c3ncn(-c4ccccc4)c3Oc3cccc(c31)O2 ; c1ccc(-c2ccc3c(c2)B2c4ncn(-c5ccccc5)c4Oc4cccc(c42)O3)cc1]

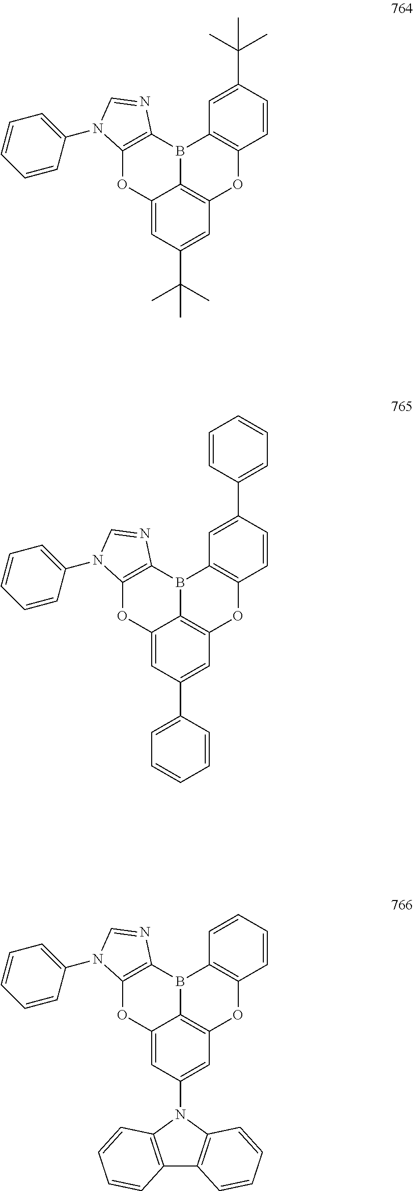 [SMILES: CC(C)(C)c1cc2c3c(c1)Oc1c(ncn1-c1ccccc1)B3c1cc(C(C)(C)C)ccc1O2 ; c1ccc(-c2cc3c4c(c2)Oc2c(ncn2-c2ccccc2)B4c2cc(-c4ccccc4)ccc2O3)cc1 ; c1ccc(-n2cnc3c2Oc2cc(-n4c5ccccc5c5ccccc54)cc4c2B3c2ccccc2O4)cc1]

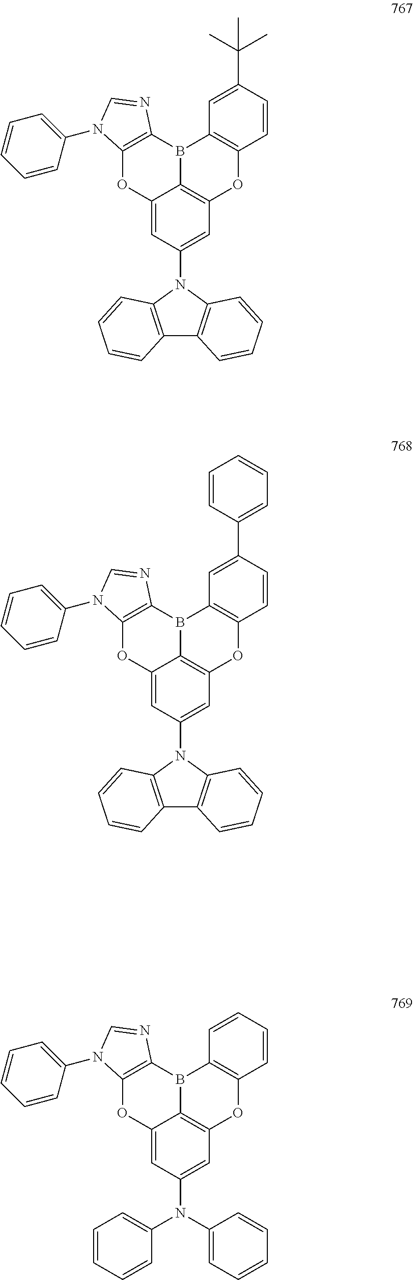 [SMILES: CC(C)(C)c1ccc2c(c1)B1c3ncn(-c4ccccc4)c3Oc3cc(-n4c5ccccc5c5ccccc54)cc(c31)O2 ; c1ccc(-c2ccc3c(c2)B2c4ncn(-c5ccccc5)c4Oc4cc(-n5c6ccccc6c6ccccc65)cc(c42)O3)cc1 ; c1ccc(N(c2ccccc2)c2cc3c4c(c2)Oc2c(ncn2-c2ccccc2)B4c2ccccc2O3)cc1]

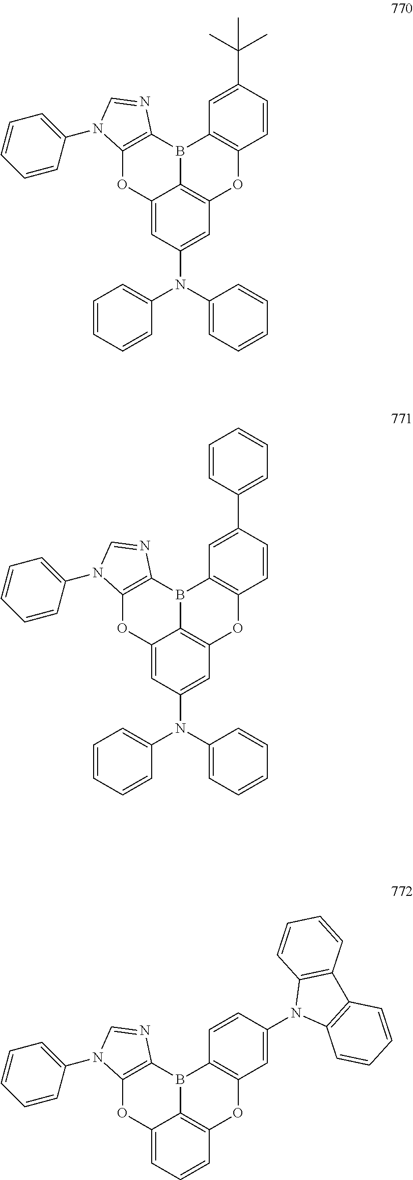 [SMILES: CC(C)(C)c1ccc2c(c1)B1c3ncn(-c4ccccc4)c3Oc3cc(N(c4ccccc4)c4ccccc4)cc(c31)O2 ; c1ccc(-c2ccc3c(c2)B2c4ncn(-c5ccccc5)c4Oc4cc(N(c5ccccc5)c5ccccc5)cc(c42)O3)cc1 ; c1ccc(-n2cnc3c2Oc2cccc4c2B3c2ccc(-n3c5ccccc5c5ccccc53)cc2O4)cc1]

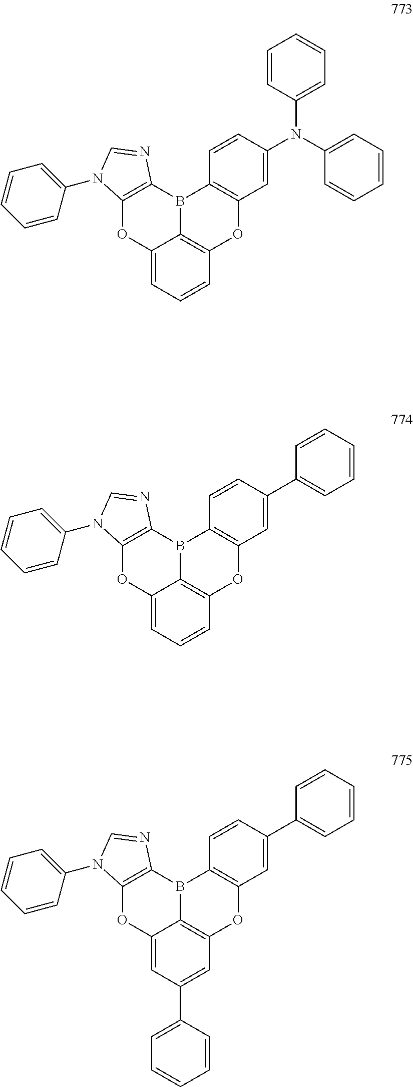 [SMILES: c1ccc(N(c2ccccc2)c2ccc3c(c2)Oc2cccc4c2B3c2ncn(-c3ccccc3)c2O4)cc1 ; c1ccc(-c2ccc3c(c2)Oc2cccc4c2B3c2ncn(-c3ccccc3)c2O4)cc1 ; c1ccc(-c2ccc3c(c2)Oc2cc(-c4ccccc4)cc4c2B3c2ncn(-c3ccccc3)c2O4)cc1]

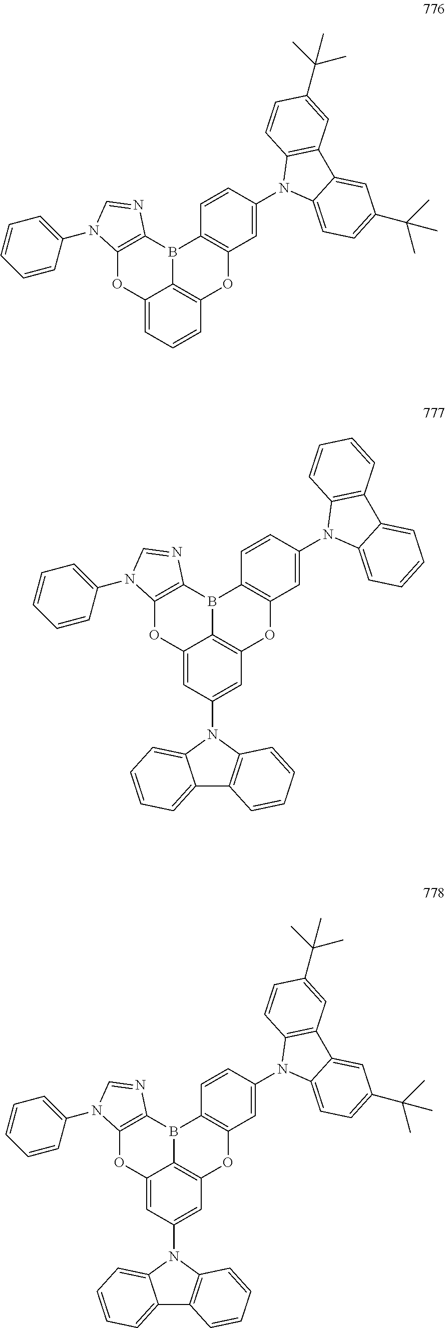 [SMILES: CC(C)(C)c1ccc2c(c1)c1cc(C(C)(C)C)ccc1n2-c1ccc2c(c1)Oc1cccc3c1B2c1ncn(-c2ccccc2)c1O3 ; c1ccc(-n2cnc3c2Oc2cc(-n4c5ccccc5c5ccccc54)cc4c2B3c2ccc(-n3c5ccccc5c5ccccc53)cc2O4)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1ncn(-c2ccccc2)c1O3]

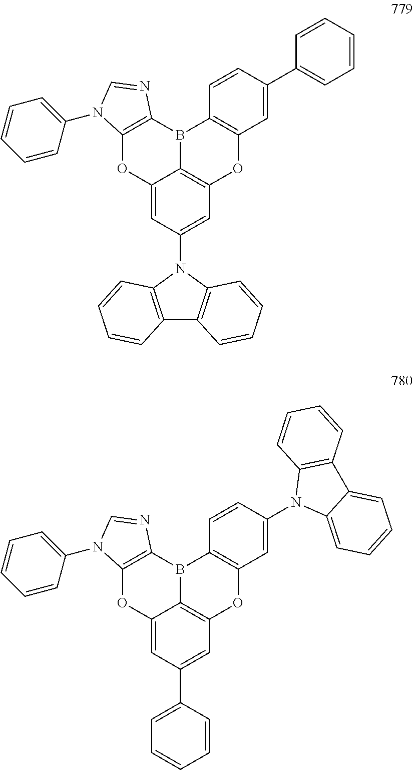 [SMILES: c1ccc(-c2ccc3c(c2)Oc2cc(-n4c5ccccc5c5ccccc54)cc4c2B3c2ncn(-c3ccccc3)c2O4)cc1 ; c1ccc(-c2cc3c4c(c2)Oc2c(ncn2-c2ccccc2)B4c2ccc(-n4c5ccccc5c5ccccc54)cc2O3)cc1]

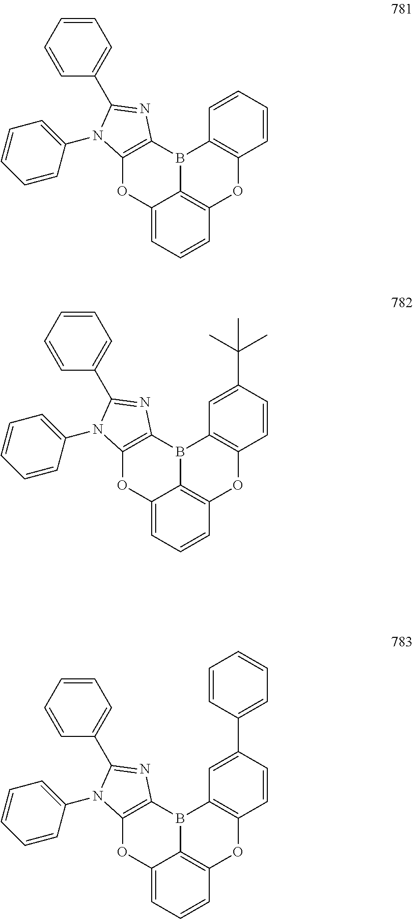 [SMILES: c1ccc(-c2nc3c(n2-c2ccccc2)Oc2cccc4c2B3c2ccccc2O4)cc1 ; CC(C)(C)c1ccc2c(c1)B1c3nc(-c4ccccc4)n(-c4ccccc4)c3Oc3cccc(c31)O2 ; c1ccc(-c2ccc3c(c2)B2c4nc(-c5ccccc5)n(-c5ccccc5)c4Oc4cccc(c42)O3)cc1]

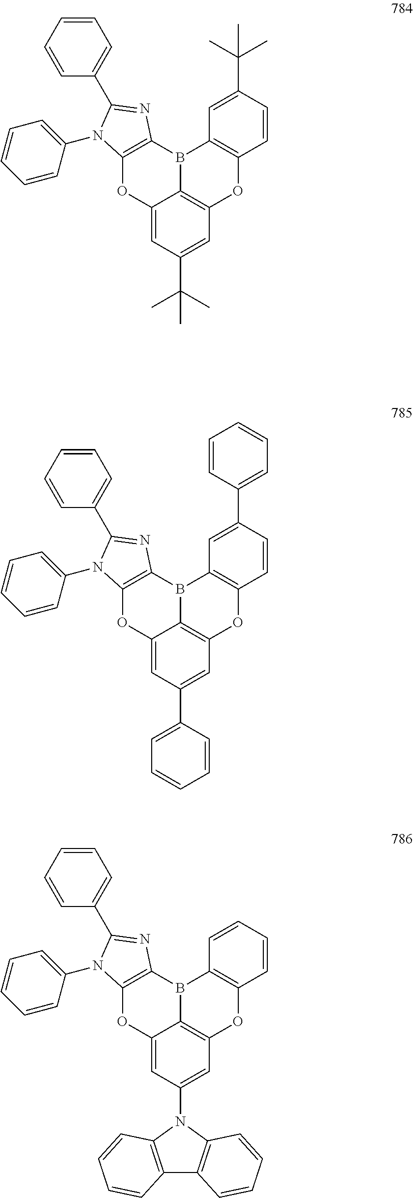 [SMILES: CC(C)(C)c1cc2c3c(c1)Oc1c(nc(-c4ccccc4)n1-c1ccccc1)B3c1cc(C(C)(C)C)ccc1O2 ; c1ccc(-c2cc3c4c(c2)Oc2c(nc(-c5ccccc5)n2-c2ccccc2)B4c2cc(-c4ccccc4)ccc2O3)cc1 ; c1ccc(-c2nc3c(n2-c2ccccc2)Oc2cc(-n4c5ccccc5c5ccccc54)cc4c2B3c2ccccc2O4)cc1]

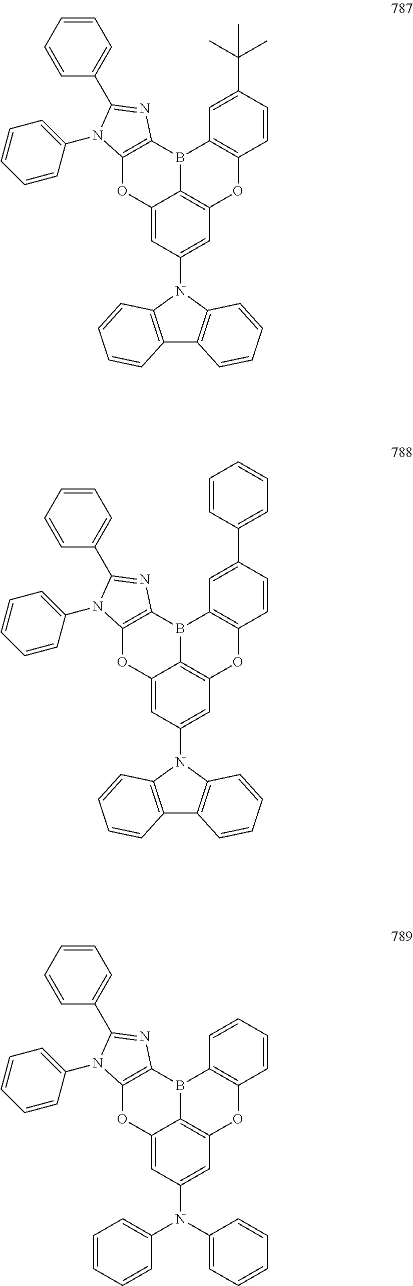 [SMILES: CC(C)(C)c1ccc2c(c1)B1c3nc(-c4ccccc4)n(-c4ccccc4)c3Oc3cc(-n4c5ccccc5c5ccccc54)cc(c31)O2 ; c1ccc(-c2ccc3c(c2)B2c4nc(-c5ccccc5)n(-c5ccccc5)c4Oc4cc(-n5c6ccccc6c6ccccc65)cc(c42)O3)cc1 ; c1ccc(-c2nc3c(n2-c2ccccc2)Oc2cc(N(c4ccccc4)c4ccccc4)cc4c2B3c2ccccc2O4)cc1]

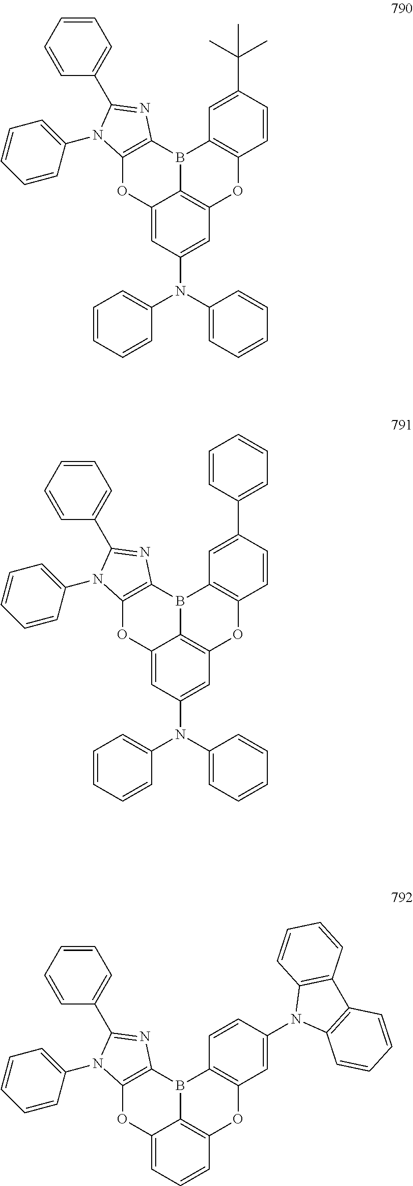 [SMILES: CC(C)(C)c1ccc2c(c1)B1c3nc(-c4ccccc4)n(-c4ccccc4)c3Oc3cc(N(c4ccccc4)c4ccccc4)cc(c31)O2 ; c1ccc(-c2ccc3c(c2)B2c4nc(-c5ccccc5)n(-c5ccccc5)c4Oc4cc(N(c5ccccc5)c5ccccc5)cc(c42)O3)cc1 ; c1ccc(-c2nc3c(n2-c2ccccc2)Oc2cccc4c2B3c2ccc(-n3c5ccccc5c5ccccc53)cc2O4)cc1]

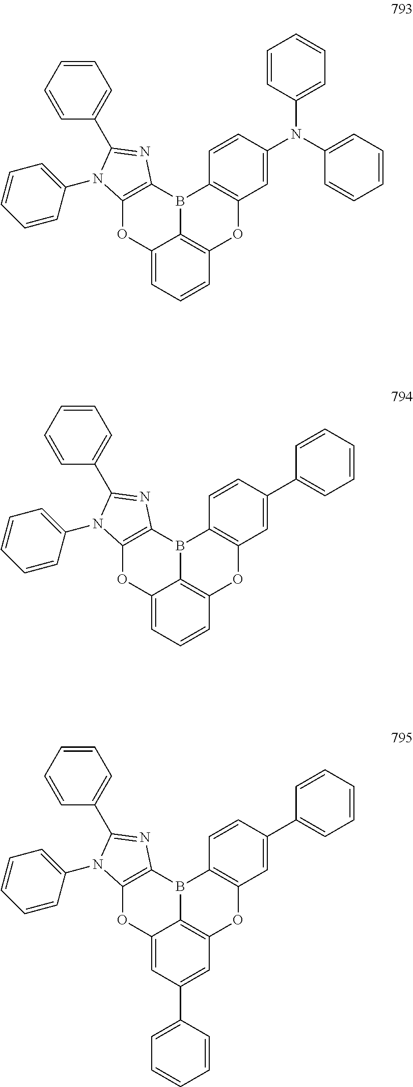 [SMILES: c1ccc(-c2nc3c(n2-c2ccccc2)Oc2cccc4c2B3c2ccc(N(c3ccccc3)c3ccccc3)cc2O4)cc1 ; c1ccc(-c2ccc3c(c2)Oc2cccc4c2B3c2nc(-c3ccccc3)n(-c3ccccc3)c2O4)cc1 ; c1ccc(-c2ccc3c(c2)Oc2cc(-c4ccccc4)cc4c2B3c2nc(-c3ccccc3)n(-c3ccccc3)c2O4)cc1]

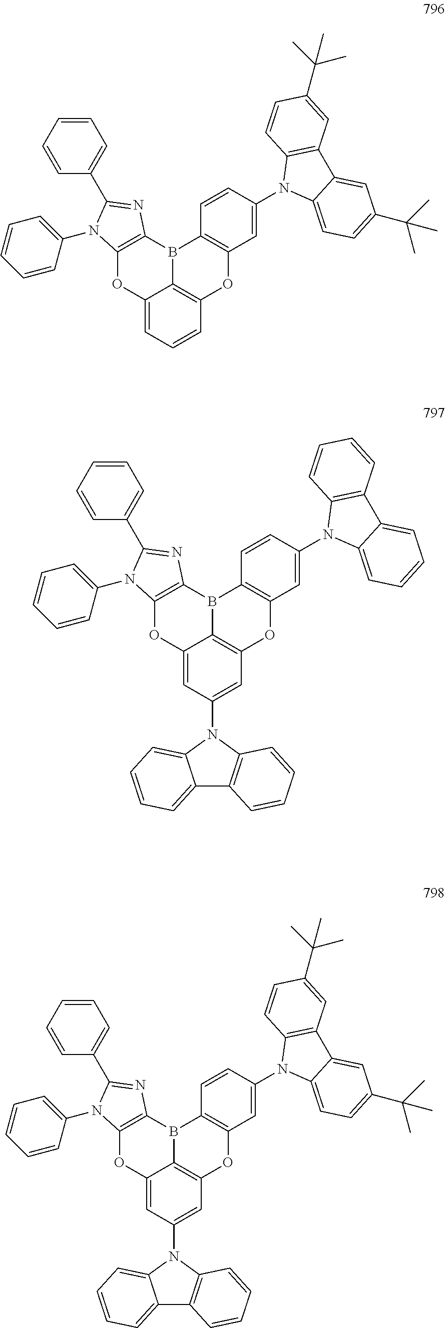 [SMILES: CC(C)(C)c1ccc2c(c1)c1cc(C(C)(C)C)ccc1n2-c1ccc2c(c1)Oc1cccc3c1B2c1nc(-c2ccccc2)n(-c2ccccc2)c1O3 ; c1ccc(-c2nc3c(n2-c2ccccc2)Oc2cc(-n4c5ccccc5c5ccccc54)cc4c2B3c2ccc(-n3c5ccccc5c5ccccc53)cc2O4)cc1 ; CC(C)(C)c1ccc2c(c1)c1cc(C(C)(C)C)ccc1n2-c1ccc2c(c1)Oc1cc(-n3c4ccccc4c4ccccc43)cc3c1B2c1nc(-c2ccccc2)n(-c2ccccc2)c1O3]

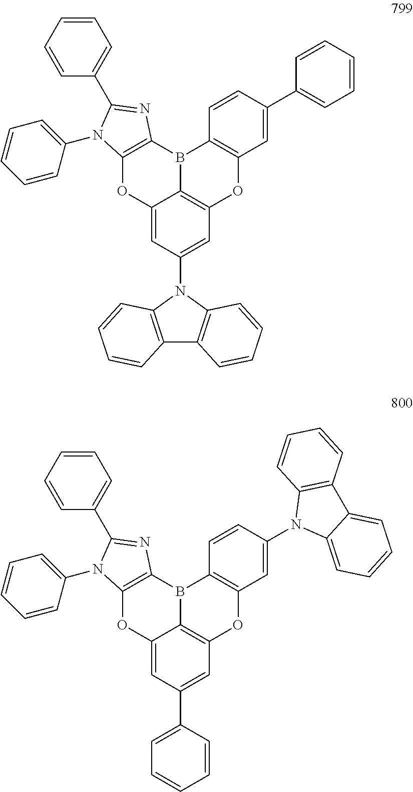 [SMILES: c1ccc(-c2ccc3c(c2)Oc2cc(-n4c5ccccc5c5ccccc54)cc4c2B3c2nc(-c3ccccc3)n(-c3ccccc3)c2O4)cc1 ; c1ccc(-c2cc3c4c(c2)Oc2c(nc(-c5ccccc5)n2-c2ccccc2)B4c2ccc(-n4c5ccccc5c5ccccc54)cc2O3)cc1]

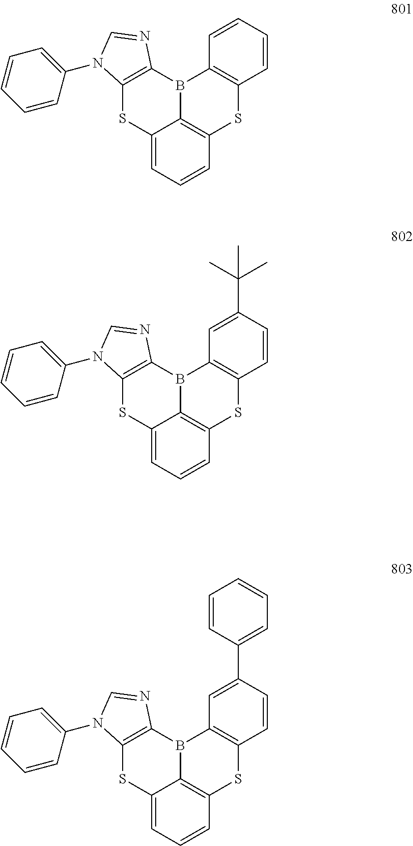 [SMILES: c1ccc(-n2cnc3c2Sc2cccc4c2B3c2ccccc2S4)cc1 ; CC(C)(C)c1ccc2c(c1)B1c3ncn(-c4ccccc4)c3Sc3cccc(c31)S2 ; c1ccc(-c2ccc3c(c2)B2c4ncn(-c5ccccc5)c4Sc4cccc(c42)S3)cc1]

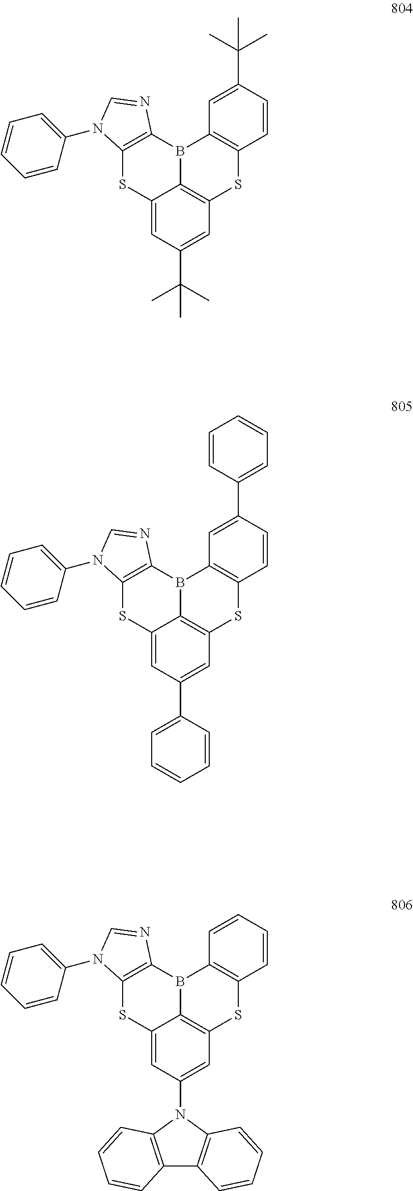 [SMILES: CC(C)(C)c1cc2c3c(c1)Sc1c(ncn1-c1ccccc1)B3c1cc(C(C)(C)C)ccc1S2 ; c1ccc(-c2cc3c4c(c2)Sc2c(ncn2-c2ccccc2)B4c2cc(-c4ccccc4)ccc2S3)cc1 ; c1ccc(-n2cnc3c2Sc2cc(-n4c5ccccc5c5ccccc54)cc4c2B3c2ccccc2S4)cc1]

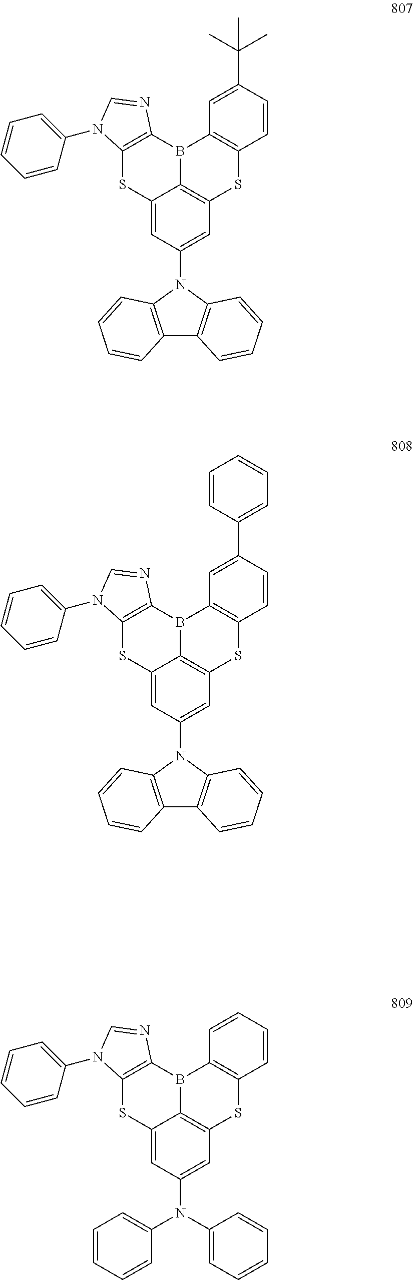 [SMILES: CC(C)(C)c1ccc2c(c1)B1c3ncn(-c4ccccc4)c3Sc3cc(-n4c5ccccc5c5ccccc54)cc(c31)S2 ; c1ccc(-c2ccc3c(c2)B2c4ncn(-c5ccccc5)c4Sc4cc(-n5c6ccccc6c6ccccc65)cc(c42)S3)cc1 ; c1ccc(N(c2ccccc2)c2cc3c4c(c2)Sc2c(ncn2-c2ccccc2)B4c2ccccc2S3)cc1]

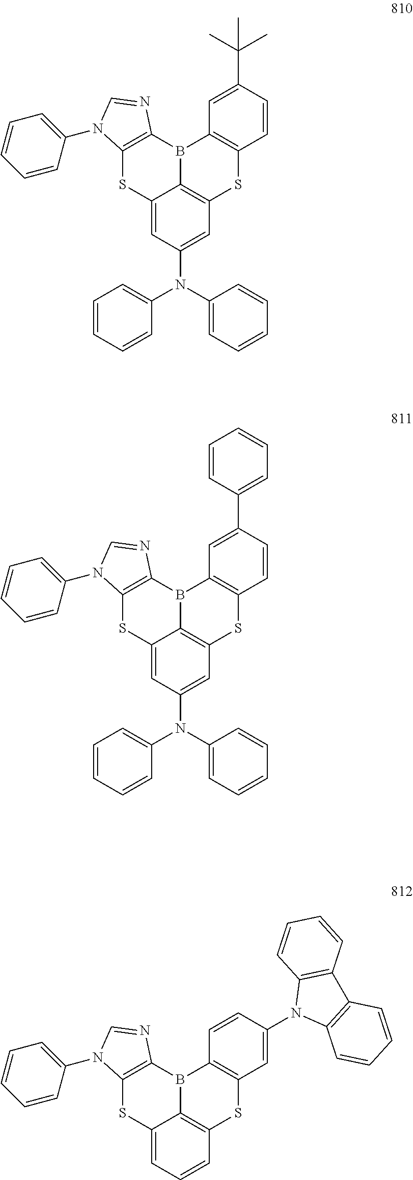 [SMILES: CC(C)(C)c1ccc2c(c1)B1c3ncn(-c4ccccc4)c3Sc3cc(N(c4ccccc4)c4ccccc4)cc(c31)S2 ; c1ccc(-c2ccc3c(c2)B2c4ncn(-c5ccccc5)c4Sc4cc(N(c5ccccc5)c5ccccc5)cc(c42)S3)cc1 ; c1ccc(-n2cnc3c2Sc2cccc4c2B3c2ccc(-n3c5ccccc5c5ccccc53)cc2S4)cc1]

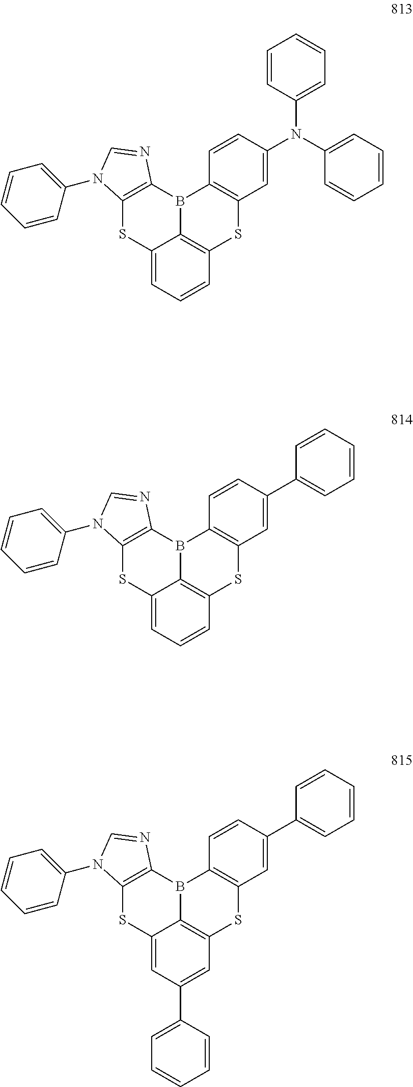 [SMILES: c1ccc(N(c2ccccc2)c2ccc3c(c2)Sc2cccc4c2B3c2ncn(-c3ccccc3)c2S4)cc1 ; c1ccc(-c2ccc3c(c2)Sc2cccc4c2B3c2ncn(-c3ccccc3)c2S4)cc1 ; c1ccc(-c2ccc3c(c2)Sc2cc(-c4ccccc4)cc4c2B3c2ncn(-c3ccccc3)c2S4)cc1]

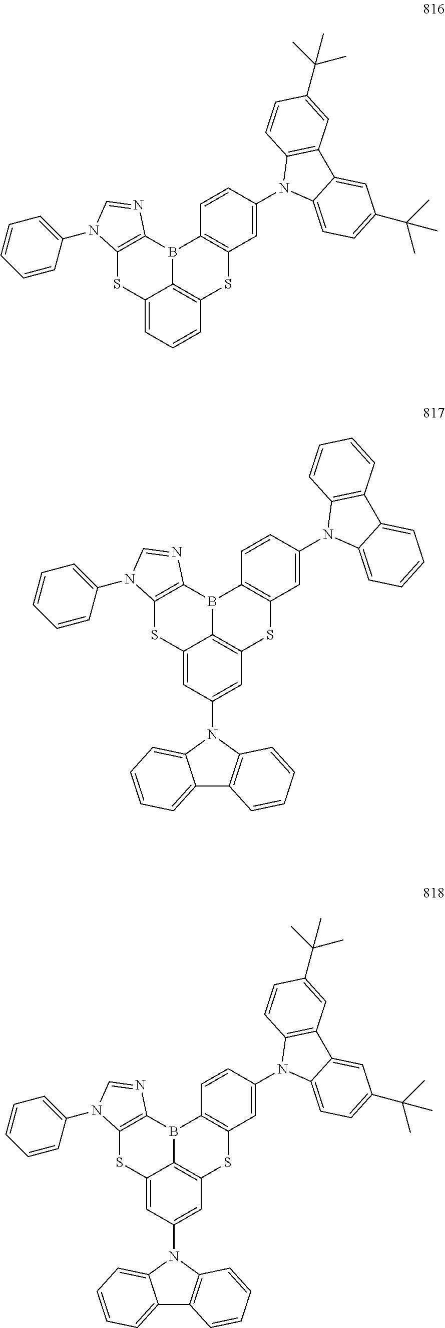 [SMILES: CC(C)(C)c1ccc2c(c1)c1cc(C(C)(C)C)ccc1n2-c1ccc2c(c1)Sc1cccc3c1B2c1ncn(-c2ccccc2)c1S3 ; c1ccc(-n2cnc3c2Sc2cc(-n4c5ccccc5c5ccccc54)cc4c2B3c2ccc(-n3c5ccccc5c5ccccc53)cc2S4)cc1 ; CC(C)(C)c1ccc2c(c1)c1cc(C(C)(C)C)ccc1n2-c1ccc2c(c1)Sc1cc(-n3c4ccccc4c4ccccc43)cc3c1B2c1ncn(-c2ccccc2)c1S3]

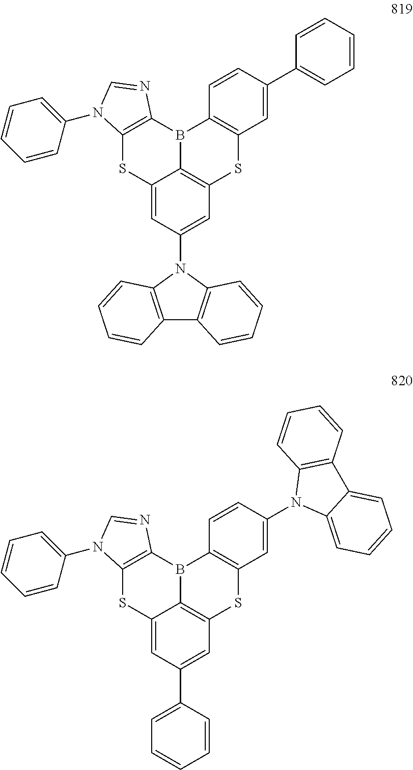 [SMILES: c1ccc(-c2ccc3c(c2)Sc2cc(-n4c5ccccc5c5ccccc54)cc4c2B3c2ncn(-c3ccccc3)c2S4)cc1 ; c1ccc(-c2cc3c4c(c2)Sc2c(ncn2-c2ccccc2)B4c2ccc(-n4c5ccccc5c5ccccc54)cc2S3)cc1]

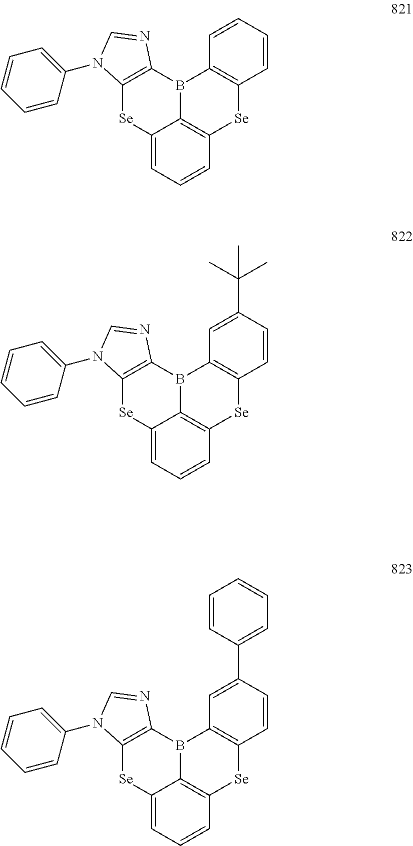 [SMILES: c1ccc(-n2cnc3c2[Se]c2cccc4c2B3c2ccccc2[Se]4)cc1 ; CC(C)(C)c1ccc2c(c1)B1c3ncn(-c4ccccc4)c3[Se]c3cccc(c31)[Se]2 ; c1ccc(-c2ccc3c(c2)B2c4ncn(-c5ccccc5)c4[Se]c4cccc(c42)[Se]3)cc1]

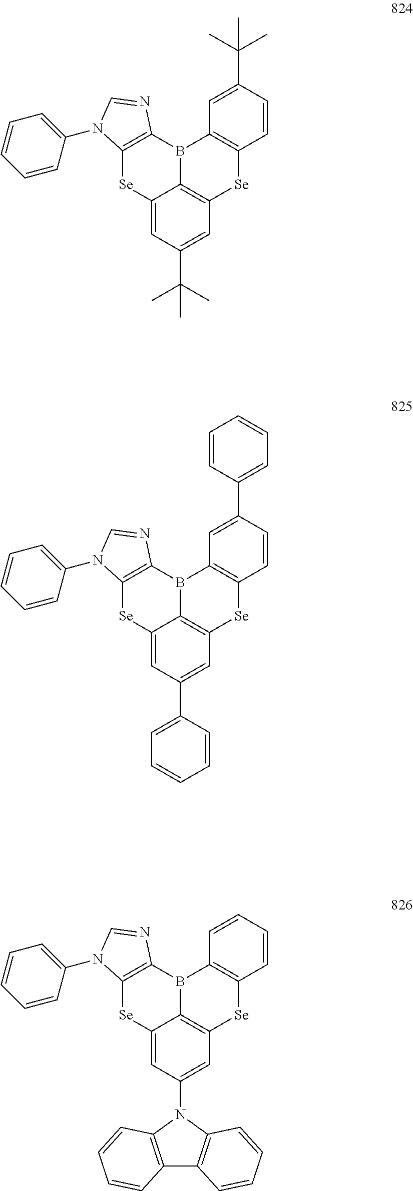 [SMILES: CC(C)(C)c1cc2c3c(c1)[Se]c1c(ncn1-c1ccccc1)B3c1cc(C(C)(C)C)ccc1[Se]2 ; c1ccc(-c2cc3c4c(c2)[Se]c2c(ncn2-c2ccccc2)B4c2cc(-c4ccccc4)ccc2[Se]3)cc1 ; c1ccc(-n2cnc3c2[Se]c2cc(-n4c5ccccc5c5ccccc54)cc4c2B3c2ccccc2[Se]4)cc1]

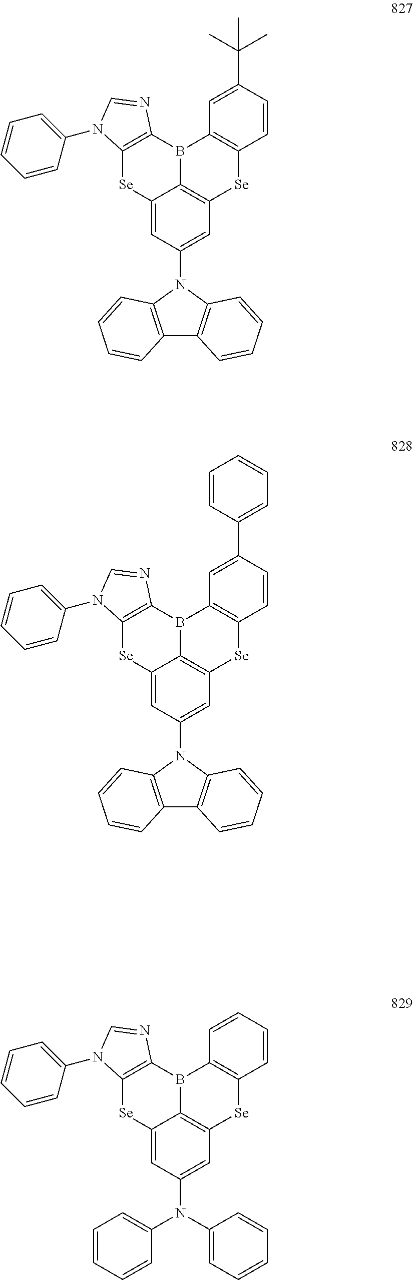 [SMILES: CC(C)(C)c1ccc2c(c1)B1c3ncn(-c4ccccc4)c3[Se]c3cc(-n4c5ccccc5c5ccccc54)cc(c31)[Se]2 ; c1ccc(-c2ccc3c(c2)B2c4ncn(-c5ccccc5)c4[Se]c4cc(-n5c6ccccc6c6ccccc65)cc(c42)[Se]3)cc1 ; c1ccc(N(c2ccccc2)c2cc3c4c(c2)[Se]c2c(ncn2-c2ccccc2)B4c2ccccc2[Se]3)cc1]

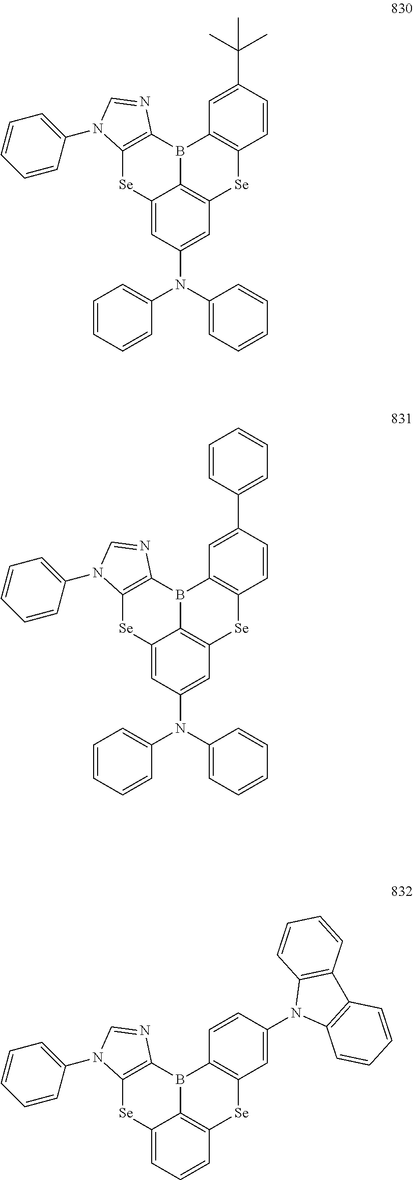 [SMILES: CC(C)(C)c1ccc2c(c1)B1c3ncn(-c4ccccc4)c3[Se]c3cc(N(c4ccccc4)c4ccccc4)cc(c31)[Se]2 ; c1ccc(-c2ccc3c(c2)B2c4ncn(-c5ccccc5)c4[Se]c4cc(N(c5ccccc5)c5ccccc5)cc(c42)[Se]3)cc1 ; c1ccc(-n2cnc3c2[Se]c2cccc4c2B3c2ccc(-n3c5ccccc5c5ccccc53)cc2[Se]4)cc1]

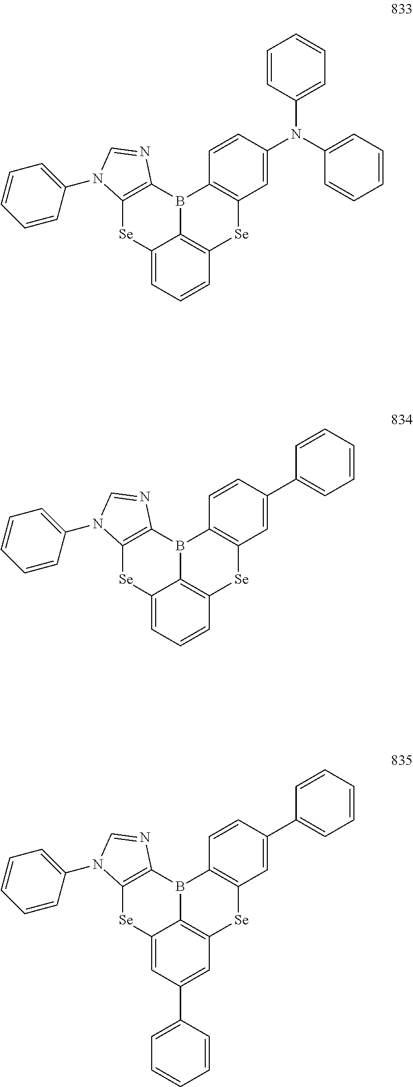 [SMILES: c1ccc(N(c2ccccc2)c2ccc3c(c2)[Se]c2cccc4c2B3c2ncn(-c3ccccc3)c2[Se]4)cc1 ; c1ccc(-c2ccc3c(c2)[Se]c2cccc4c2B3c2ncn(-c3ccccc3)c2[Se]4)cc1 ; c1ccc(-c2ccc3c(c2)[Se]c2cc(-c4ccccc4)cc4c2B3c2ncn(-c3ccccc3)c2[Se]4)cc1]

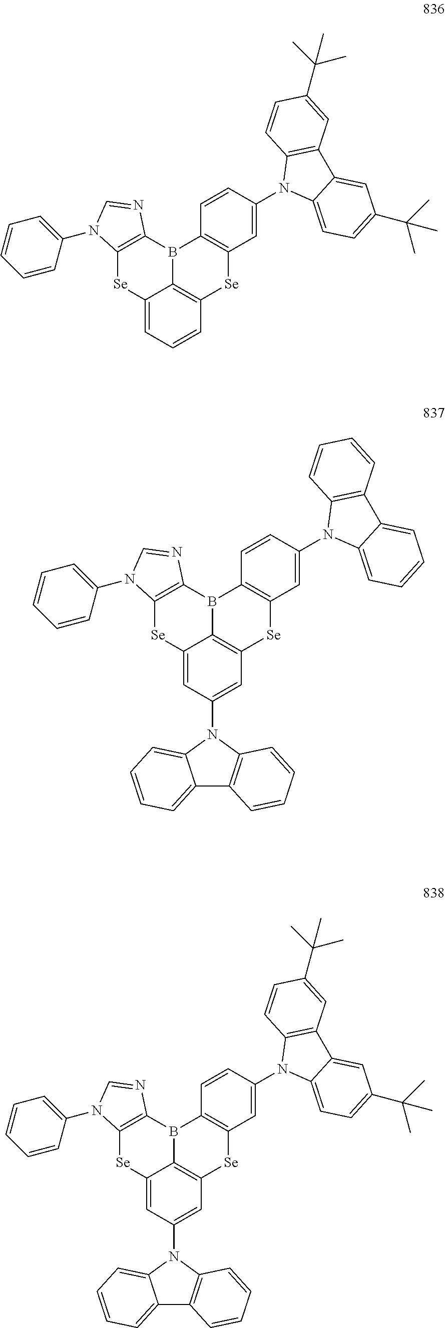 [SMILES: CC(C)(C)c1ccc2c(c1)c1cc(C(C)(C)C)ccc1n2-c1ccc2c(c1)[Se]c1cccc3c1B2c1ncn(-c2ccccc2)c1[Se]3 ; c1ccc(-n2cnc3c2[Se]c2cc(-n4c5ccccc5c5ccccc54)cc4c2B3c2ccc(-n3c5ccccc5c5ccccc53)cc2[Se]4)cc1 ; CC(C)(C)c1ccc2c(c1)c1cc(C(C)(C)C)ccc1n2-c1ccc2c(c1)[Se]c1cc(-n3c4ccccc4c4ccccc43)cc3c1B2c1ncn(-c2ccccc2)c1[Se]3]

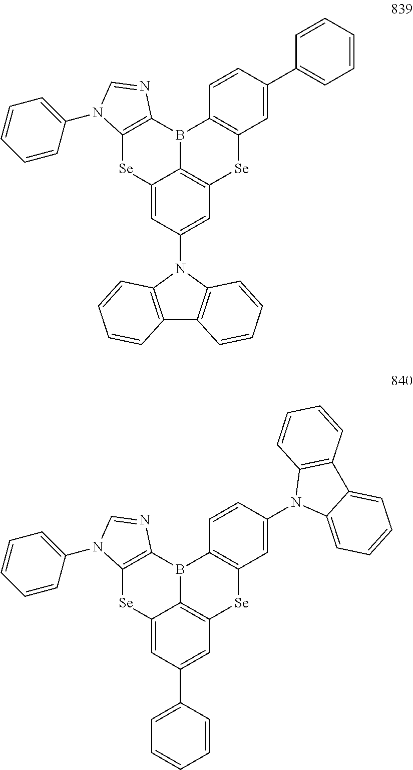 [SMILES: c1ccc(-c2ccc3c(c2)[Se]c2cc(-n4c5ccccc5c5ccccc54)cc4c2B3c2ncn(-c3ccccc3)c2[Se]4)cc1 ; c1ccc(-c2cc3c4c(c2)[Se]c2c(ncn2-c2ccccc2)B4c2ccc(-n4c5ccccc5c5ccccc54)cc2[Se]3)cc1]

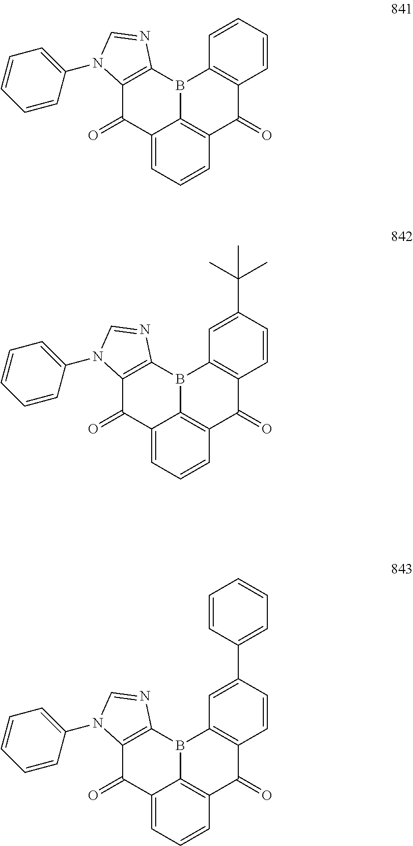 [SMILES: O=C1c2ccccc2B2c3ncn(-c4ccccc4)c3C(=O)c3cccc1c32 ; CC(C)(C)c1ccc2c(c1)B1c3ncn(-c4ccccc4)c3C(=O)c3cccc(c31)C2=O ; O=C1c2ccc(-c3ccccc3)cc2B2c3ncn(-c4ccccc4)c3C(=O)c3cccc1c32]

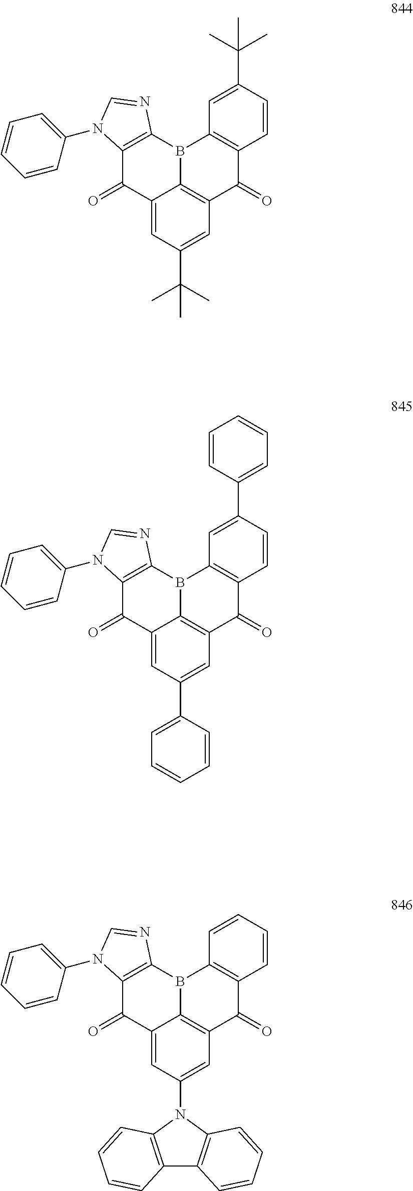 [SMILES: CC(C)(C)c1ccc2c(c1)B1c3ncn(-c4ccccc4)c3C(=O)c3cc(C(C)(C)C)cc(c31)C2=O ; O=C1c2ccc(-c3ccccc3)cc2B2c3ncn(-c4ccccc4)c3C(=O)c3cc(-c4ccccc4)cc1c32 ; O=C1c2ccccc2B2c3ncn(-c4ccccc4)c3C(=O)c3cc(-n4c5ccccc5c5ccccc54)cc1c32]

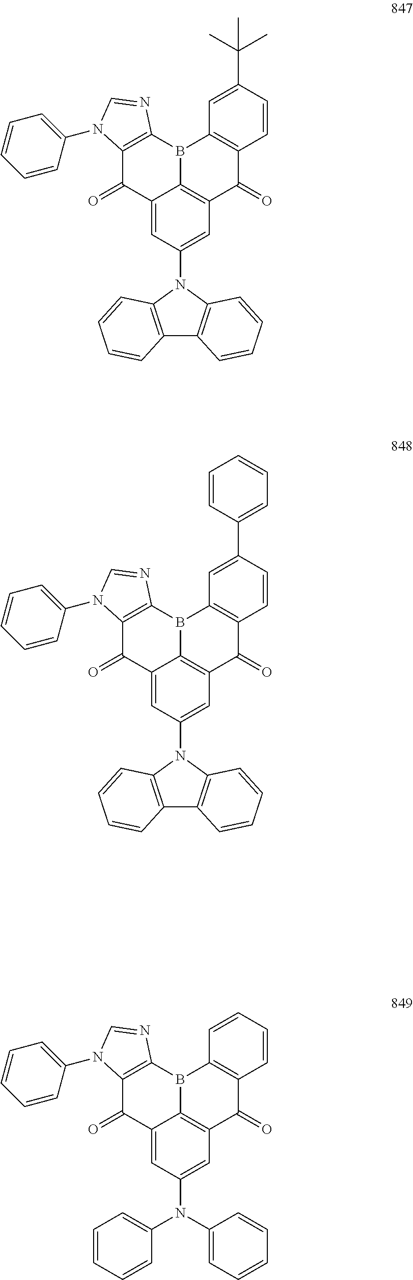 [SMILES: CC(C)(C)c1ccc2c(c1)B1c3ncn(-c4ccccc4)c3C(=O)c3cc(-n4c5ccccc5c5ccccc54)cc(c31)C2=O ; O=C1c2ccc(-c3ccccc3)cc2B2c3ncn(-c4ccccc4)c3C(=O)c3cc(-n4c5ccccc5c5ccccc54)cc1c32 ; O=C1c2ccccc2B2c3ncn(-c4ccccc4)c3C(=O)c3cc(N(c4ccccc4)c4ccccc4)cc1c32]

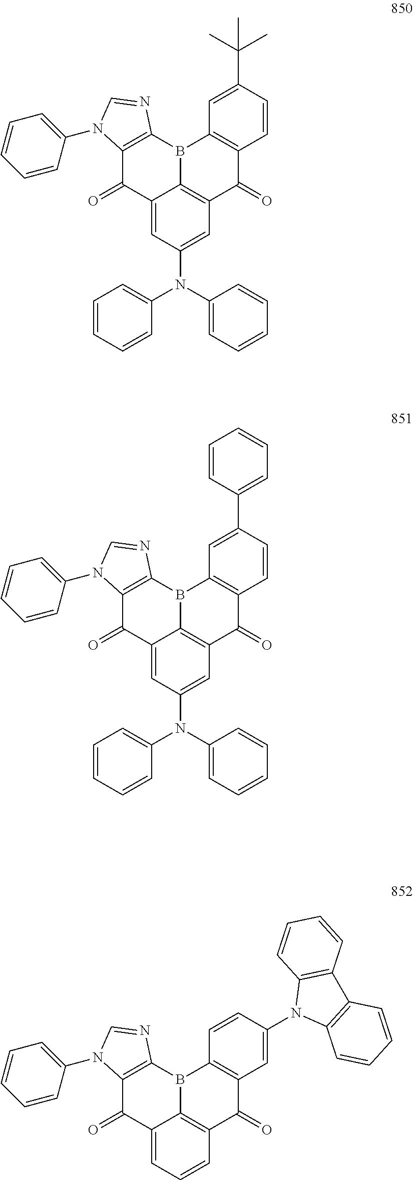 [SMILES: CC(C)(C)c1ccc2c(c1)B1c3ncn(-c4ccccc4)c3C(=O)c3cc(N(c4ccccc4)c4ccccc4)cc(c31)C2=O ; O=C1c2ccc(-c3ccccc3)cc2B2c3ncn(-c4ccccc4)c3C(=O)c3cc(N(c4ccccc4)c4ccccc4)cc1c32 ; O=C1c2cc(-n3c4ccccc4c4ccccc43)ccc2B2c3ncn(-c4ccccc4)c3C(=O)c3cccc1c32]

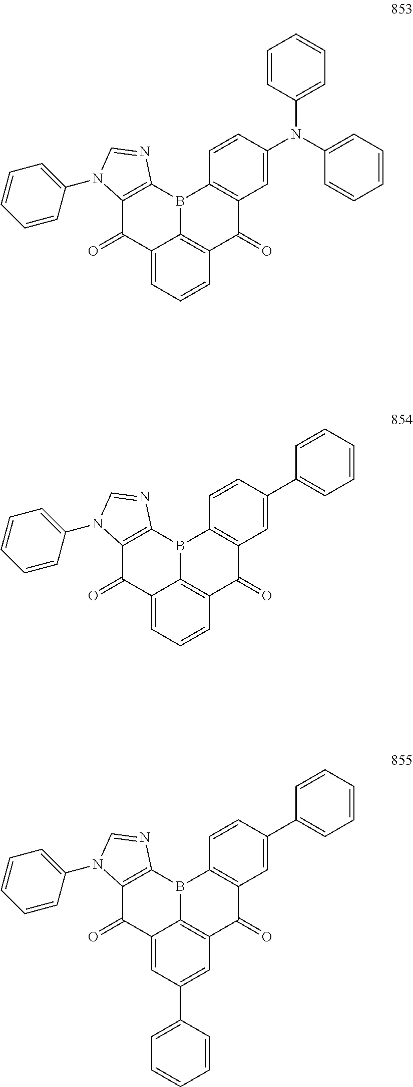 [SMILES: O=C1c2cc(N(c3ccccc3)c3ccccc3)ccc2B2c3ncn(-c4ccccc4)c3C(=O)c3cccc1c32 ; O=C1c2cc(-c3ccccc3)ccc2B2c3ncn(-c4ccccc4)c3C(=O)c3cccc1c32 ; O=C1c2cc(-c3ccccc3)ccc2B2c3ncn(-c4ccccc4)c3C(=O)c3cc(-c4ccccc4)cc1c32]

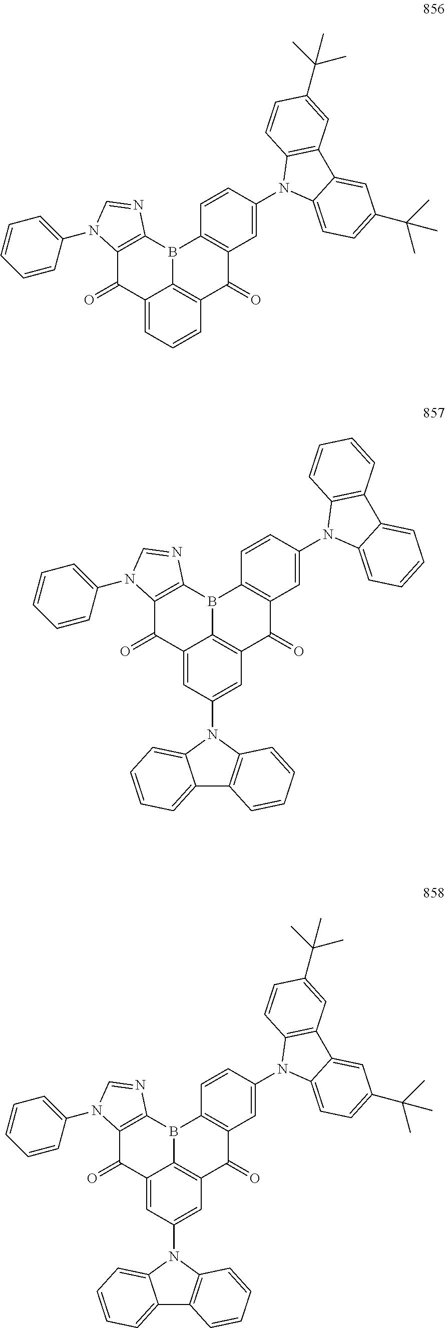 [SMILES: CC(C)(C)c1ccc2c(c1)c1cc(C(C)(C)C)ccc1n2-c1ccc2c(c1)C(=O)c1cccc3c1B2c1ncn(-c2ccccc2)c1C3=O ; O=C1c2cc(-n3c4ccccc4c4ccccc43)ccc2B2c3ncn(-c4ccccc4)c3C(=O)c3cc(-n4c5ccccc5c5ccccc54)cc1c32 ; CC(C)(C)c1ccc2c(c1)c1cc(C(C)(C)C)ccc1n2-c1ccc2c(c1)C(=O)c1cc(-n3c4ccccc4c4ccccc43)cc3c1B2c1ncn(-c2ccccc2)c1C3=O]

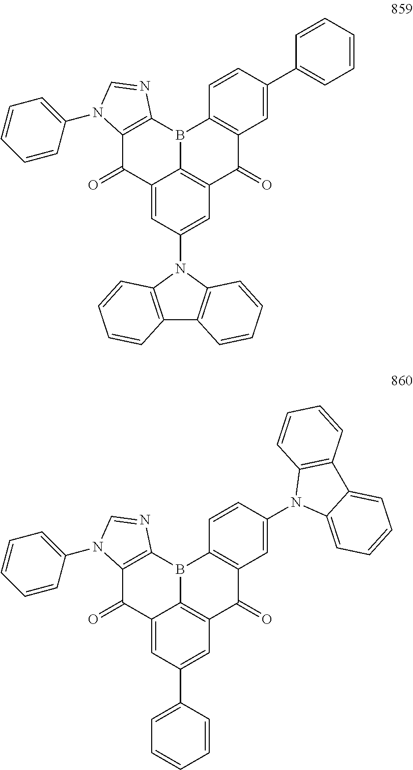 [SMILES: O=C1c2cc(-c3ccccc3)ccc2B2c3ncn(-c4ccccc4)c3C(=O)c3cc(-n4c5ccccc5c5ccccc54)cc1c32 ; O=C1c2cc(-n3c4ccccc4c4ccccc43)ccc2B2c3ncn(-c4ccccc4)c3C(=O)c3cc(-c4ccccc4)cc1c32]

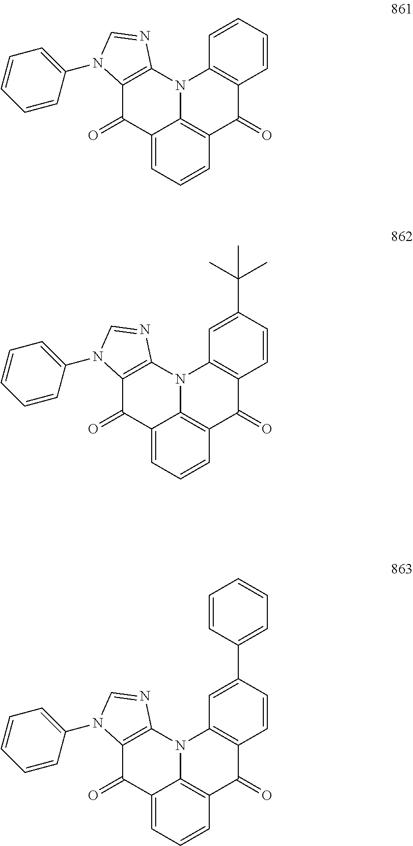 [SMILES: O=c1c2ccccc2n2c3ncn(-c4ccccc4)c3c(=O)c3cccc1c32 ; CC(C)(C)c1ccc2c(=O)c3cccc4c(=O)c5c(ncn5-c5ccccc5)n(c2c1)c34 ; O=c1c2ccc(-c3ccccc3)cc2n2c3ncn(-c4ccccc4)c3c(=O)c3cccc1c32]

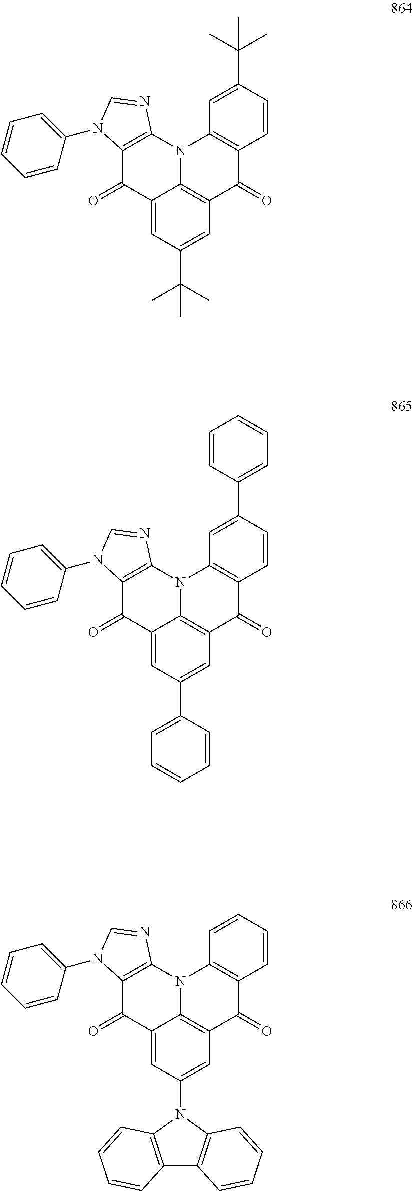 [SMILES: CC(C)(C)c1cc2c(=O)c3ccc(C(C)(C)C)cc3n3c4ncn(-c5ccccc5)c4c(=O)c(c1)c23 ; O=c1c2ccc(-c3ccccc3)cc2n2c3ncn(-c4ccccc4)c3c(=O)c3cc(-c4ccccc4)cc1c32 ; O=c1c2ccccc2n2c3ncn(-c4ccccc4)c3c(=O)c3cc(-n4c5ccccc5c5ccccc54)cc1c32]

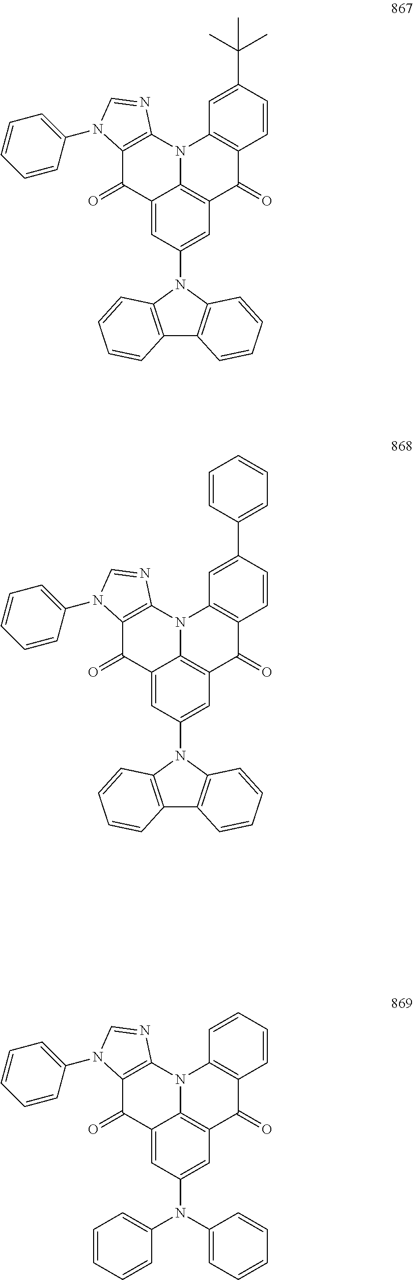 [SMILES: CC(C)(C)c1ccc2c(=O)c3cc(-n4c5ccccc5c5ccccc54)cc4c(=O)c5c(ncn5-c5ccccc5)n(c2c1)c34 ; O=c1c2ccc(-c3ccccc3)cc2n2c3ncn(-c4ccccc4)c3c(=O)c3cc(-n4c5ccccc5c5ccccc54)cc1c32 ; O=c1c2ccccc2n2c3ncn(-c4ccccc4)c3c(=O)c3cc(N(c4ccccc4)c4ccccc4)cc1c32]

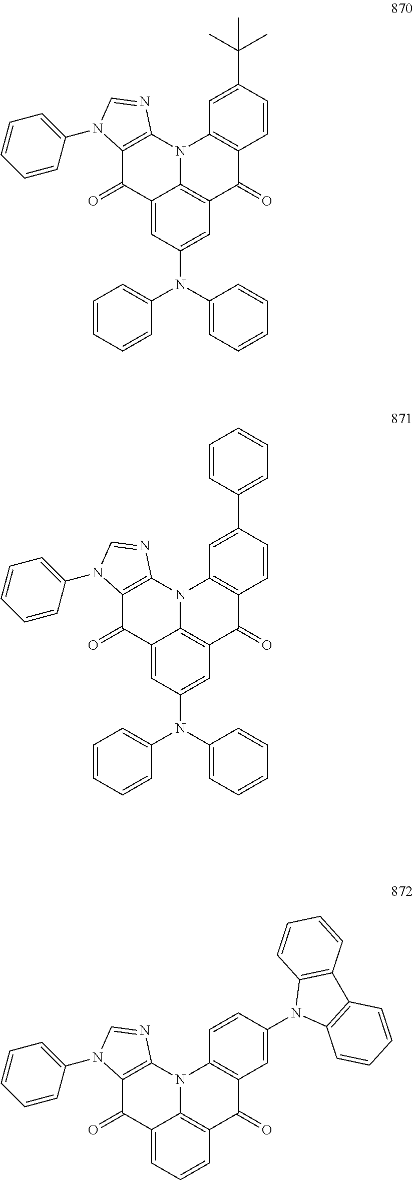 [SMILES: CC(C)(C)c1ccc2c(=O)c3cc(N(c4ccccc4)c4ccccc4)cc4c(=O)c5c(ncn5-c5ccccc5)n(c2c1)c34 ; O=c1c2ccc(-c3ccccc3)cc2n2c3ncn(-c4ccccc4)c3c(=O)c3cc(N(c4ccccc4)c4ccccc4)cc1c32 ; O=c1c2cc(-n3c4ccccc4c4ccccc43)ccc2n2c3ncn(-c4ccccc4)c3c(=O)c3cccc1c32]

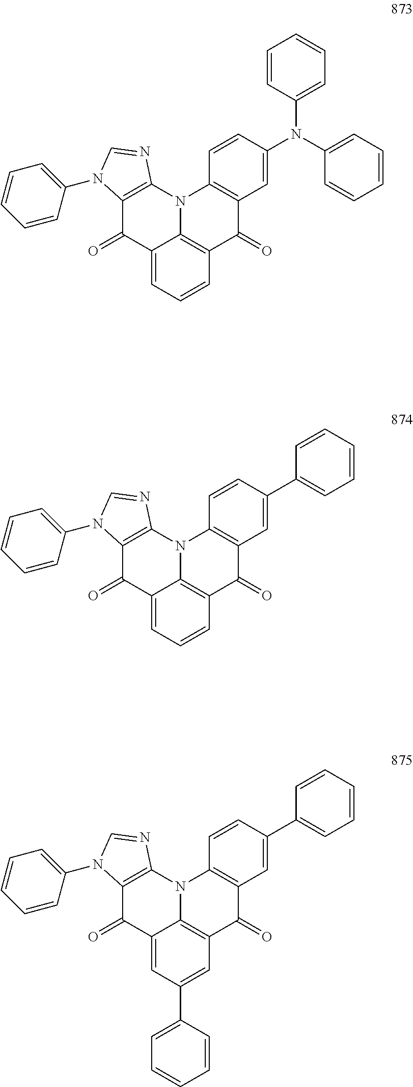 [SMILES: O=c1c2cc(N(c3ccccc3)c3ccccc3)ccc2n2c3ncn(-c4ccccc4)c3c(=O)c3cccc1c32 ; O=c1c2cc(-c3ccccc3)ccc2n2c3ncn(-c4ccccc4)c3c(=O)c3cccc1c32 ; O=c1c2cc(-c3ccccc3)ccc2n2c3ncn(-c4ccccc4)c3c(=O)c3cc(-c4ccccc4)cc1c32]

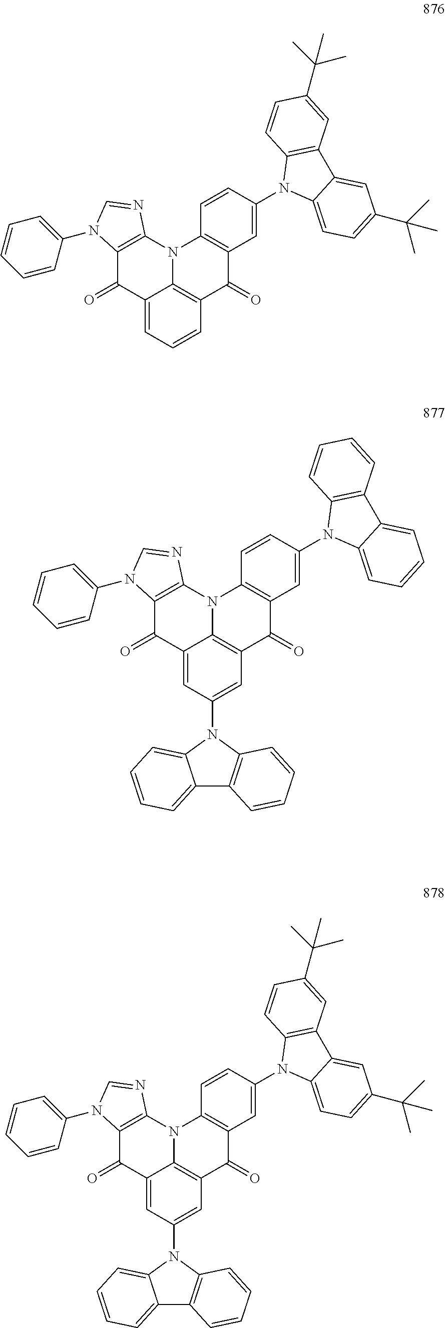 [SMILES: CC(C)(C)c1ccc2c(c1)c1cc(C(C)(C)C)ccc1n2-c1ccc2c(c1)c(=O)c1cccc3c(=O)c4c(ncn4-c4ccccc4)n2c13 ; O=c1c2cc(-n3c4ccccc4c4ccccc43)ccc2n2c3ncn(-c4ccccc4)c3c(=O)c3cc(-n4c5ccccc5c5ccccc54)cc1c32 ; CC(C)(C)c1ccc2c(c1)c1cc(C(C)(C)C)ccc1n2-c1ccc2c(c1)c(=O)c1cc(-n3c4ccccc4c4ccccc43)cc3c(=O)c4c(ncn4-c4ccccc4)n2c13]

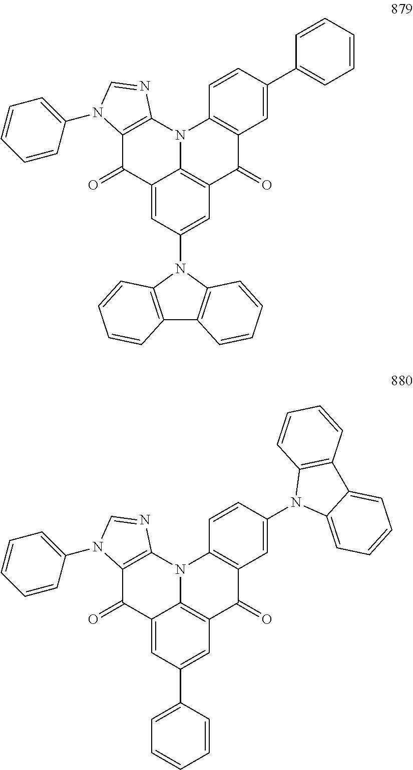 [SMILES: O=c1c2cc(-c3ccccc3)ccc2n2c3ncn(-c4ccccc4)c3c(=O)c3cc(-n4c5ccccc5c5ccccc54)cc1c32 ; O=c1c2cc(-n3c4ccccc4c4ccccc43)ccc2n2c3ncn(-c4ccccc4)c3c(=O)c3cc(-c4ccccc4)cc1c32]

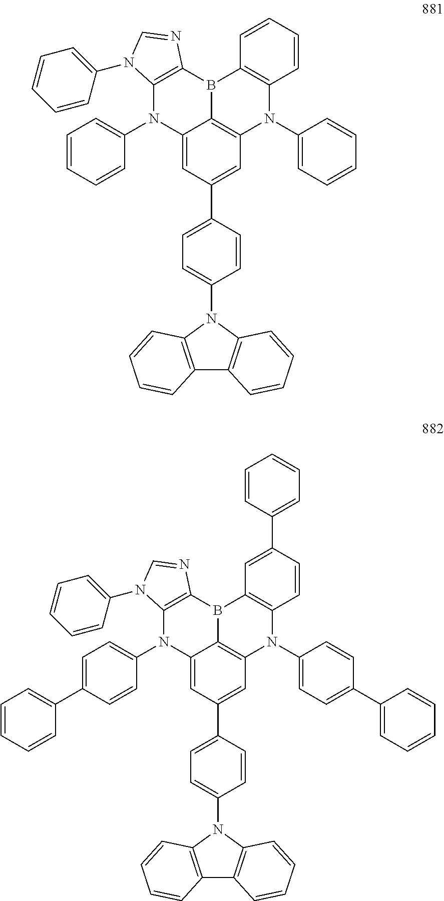 [SMILES: c1ccc(N2c3ccccc3B3c4ncn(-c5ccccc5)c4N(c4ccccc4)c4cc(-c5ccc(-n6c7ccccc7c7ccccc76)cc5)cc2c43)cc1 ; c1ccc(-c2ccc(N3c4ccc(-c5ccccc5)cc4B4c5ncn(-c6ccccc6)c5N(c5ccc(-c6ccccc6)cc5)c5cc(-c6ccc(-n7c8ccccc8c8ccccc87)cc6)cc3c54)cc2)cc1]

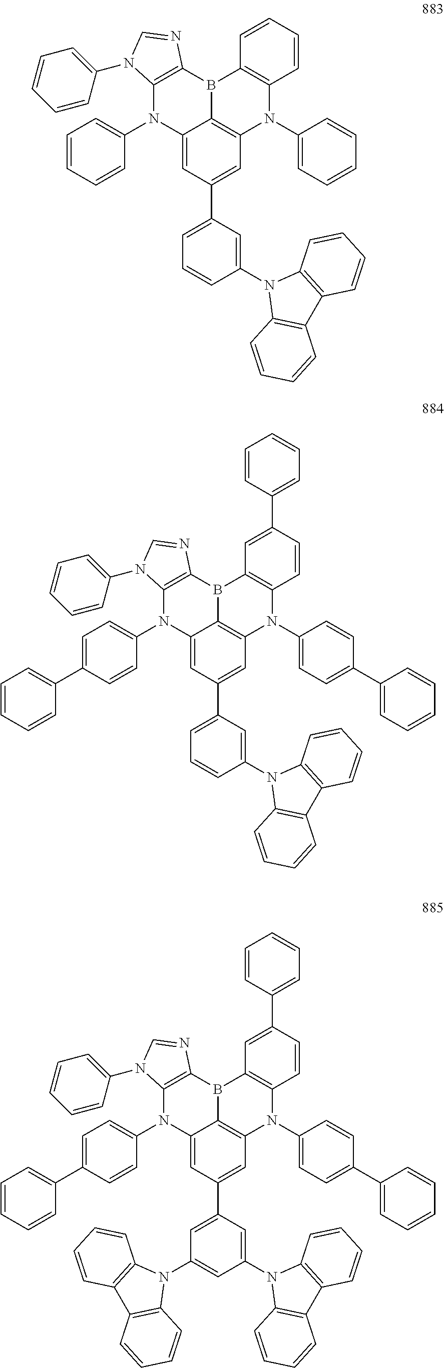 [SMILES: c1ccc(N2c3ccccc3B3c4ncn(-c5ccccc5)c4N(c4ccccc4)c4cc(-c5cccc(-n6c7ccccc7c7ccccc76)c5)cc2c43)cc1 ; c1ccc(-c2ccc(N3c4ccc(-c5ccccc5)cc4B4c5ncn(-c6ccccc6)c5N(c5ccc(-c6ccccc6)cc5)c5cc(-c6cccc(-n7c8ccccc8c8ccccc87)c6)cc3c54)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-c6cc(-n7c8ccccc8c8ccccc87)cc(-n7c8ccccc8c8ccccc87)c6)cc3c54)cc2)cc1]

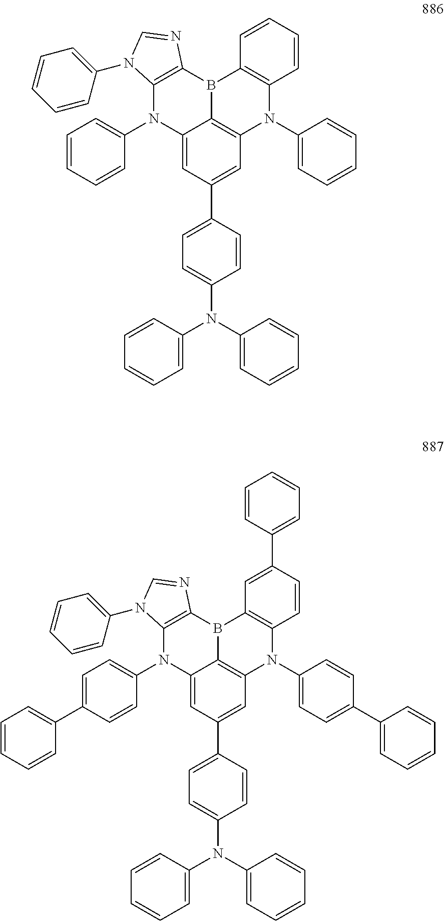 [SMILES: c1ccc(N(c2ccccc2)c2ccc(-c3cc4c5c(c3)N(c3ccccc3)c3c(ncn3-c3ccccc3)B5c3ccccc3N4c3ccccc3)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-c6ccc(N(c7ccccc7)c7ccccc7)cc6)cc3c54)cc2)cc1]

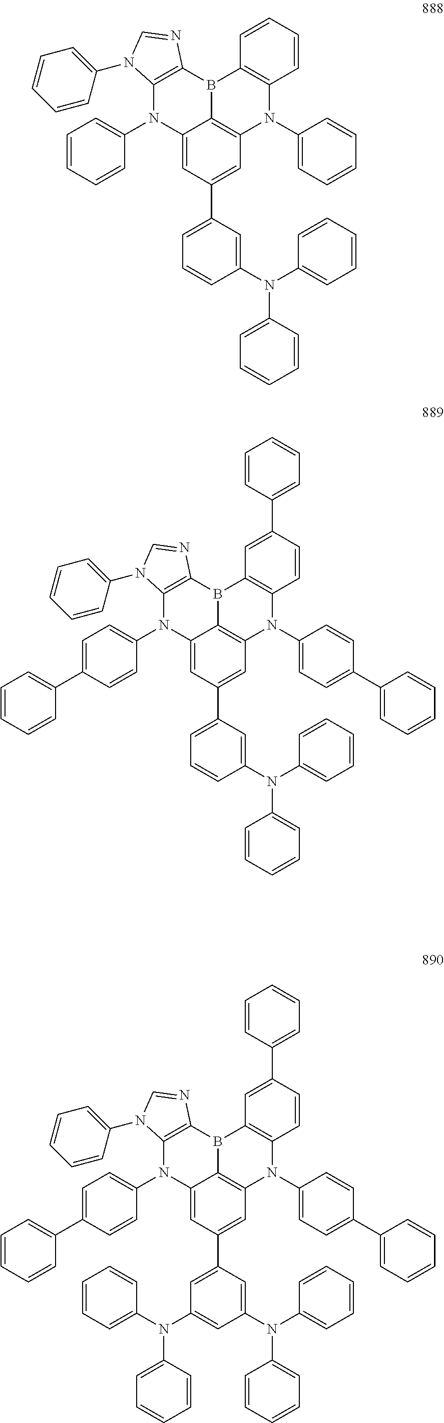 [SMILES: c1ccc(N(c2ccccc2)c2cccc(-c3cc4c5c(c3)N(c3ccccc3)c3c(ncn3-c3ccccc3)B5c3ccccc3N4c3ccccc3)c2)cc1 ; c1ccc(-c2ccc(N3c4ccc(-c5ccccc5)cc4B4c5ncn(-c6ccccc6)c5N(c5ccc(-c6ccccc6)cc5)c5cc(-c6cccc(N(c7ccccc7)c7ccccc7)c6)cc3c54)cc2)cc1 ; c1ccc(-c2ccc(N3c4ccc(-c5ccccc5)cc4B4c5ncn(-c6ccccc6)c5N(c5ccc(-c6ccccc6)cc5)c5cc(-c6cc(N(c7ccccc7)c7ccccc7)cc(N(c7ccccc7)c7ccccc7)c6)cc3c54)cc2)cc1]

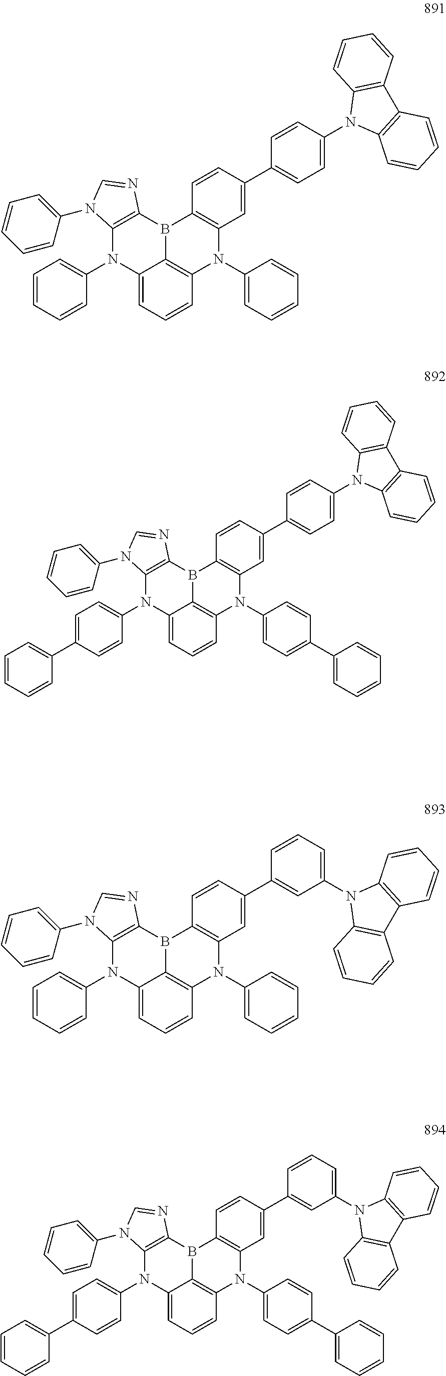 [SMILES: c1ccc(N2c3cc(-c4ccc(-n5c6ccccc6c6ccccc65)cc4)ccc3B3c4ncn(-c5ccccc5)c4N(c4ccccc4)c4cccc2c43)cc1 ; c1ccc(-c2ccc(N3c4cc(-c5ccc(-n6c7ccccc7c7ccccc76)cc5)ccc4B4c5ncn(-c6ccccc6)c5N(c5ccc(-c6ccccc6)cc5)c5cccc3c54)cc2)cc1 ; c1ccc(N2c3cc(-c4cccc(-n5c6ccccc6c6ccccc65)c4)ccc3B3c4ncn(-c5ccccc5)c4N(c4ccccc4)c4cccc2c43)cc1 ; c1ccc(-c2ccc(N3c4cc(-c5cccc(-n6c7ccccc7c7ccccc76)c5)ccc4B4c5ncn(-c6ccccc6)c5N(c5ccc(-c6ccccc6)cc5)c5cccc3c54)cc2)cc1]

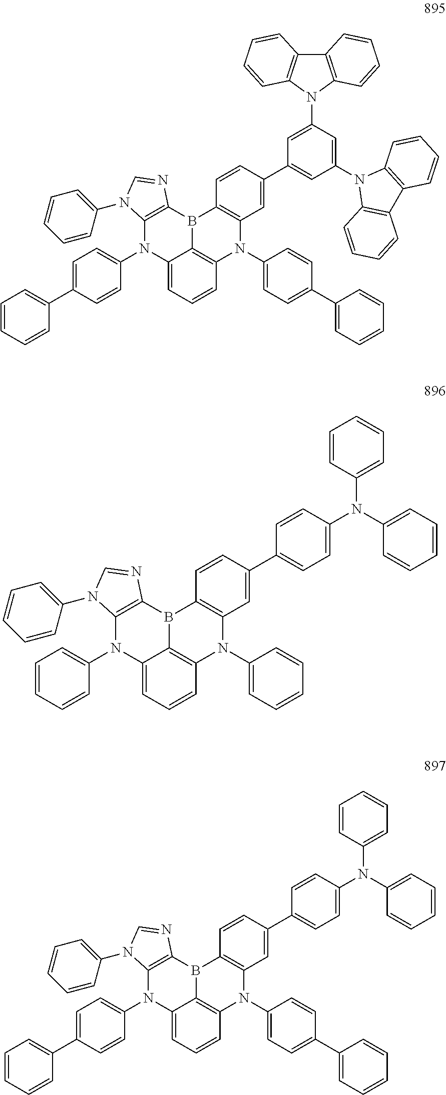 [SMILES: c1ccc(-c2ccc(N3c4cc(-c5cc(-n6c7ccccc7c7ccccc76)cc(-n6c7ccccc7c7ccccc76)c5)ccc4B4c5ncn(-c6ccccc6)c5N(c5ccc(-c6ccccc6)cc5)c5cccc3c54)cc2)cc1 ; c1ccc(N(c2ccccc2)c2ccc(-c3ccc4c(c3)N(c3ccccc3)c3cccc5c3B4c3ncn(-c4ccccc4)c3N5c3ccccc3)cc2)cc1 ; c1ccc(-c2ccc(N3c4cc(-c5ccc(N(c6ccccc6)c6ccccc6)cc5)ccc4B4c5ncn(-c6ccccc6)c5N(c5ccc(-c6ccccc6)cc5)c5cccc3c54)cc2)cc1]

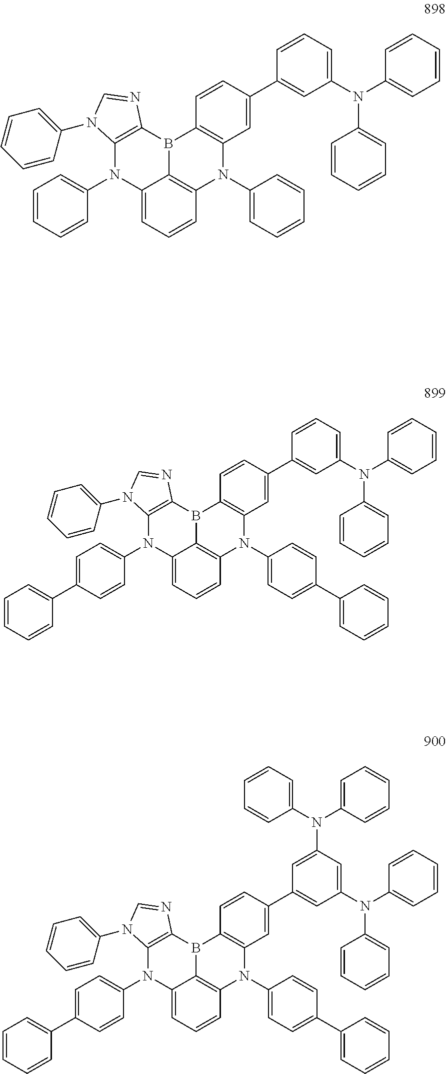 [SMILES: c1ccc(N(c2ccccc2)c2cccc(-c3ccc4c(c3)N(c3ccccc3)c3cccc5c3B4c3ncn(-c4ccccc4)c3N5c3ccccc3)c2)cc1 ; c1ccc(-c2ccc(N3c4cc(-c5cccc(N(c6ccccc6)c6ccccc6)c5)ccc4B4c5ncn(-c6ccccc6)c5N(c5ccc(-c6ccccc6)cc5)c5cccc3c54)cc2)cc1 ; c1ccc(-c2ccc(N3c4cc(-c5cc(N(c6ccccc6)c6ccccc6)cc(N(c6ccccc6)c6ccccc6)c5)ccc4B4c5ncn(-c6ccccc6)c5N(c5ccc(-c6ccccc6)cc5)c5cccc3c54)cc2)cc1]

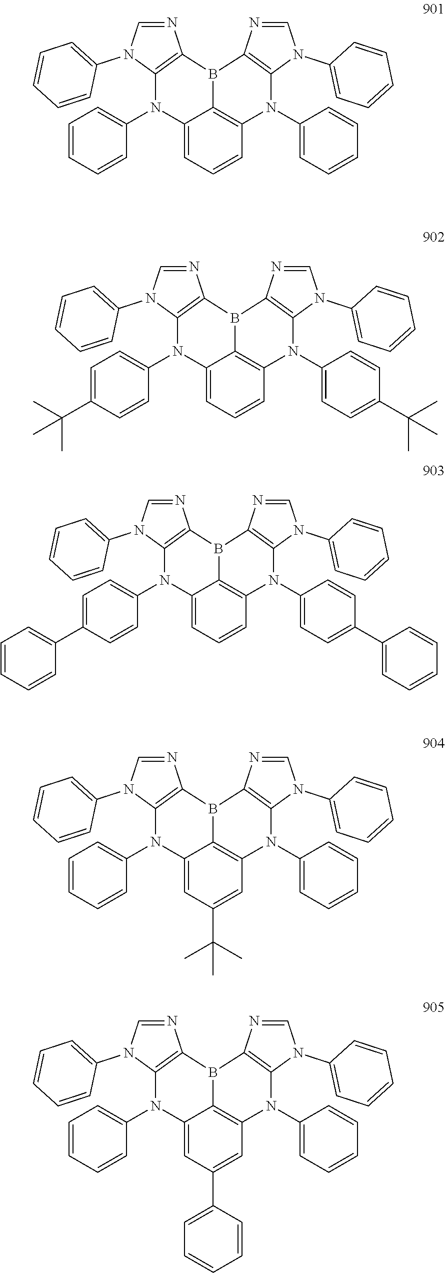 [SMILES: c1ccc(N2c3cccc4c3B(c3ncn(-c5ccccc5)c32)c2ncn(-c3ccccc3)c2N4c2ccccc2)cc1 ; CC(C)(C)c1ccc(N2c3cccc4c3B(c3ncn(-c5ccccc5)c32)c2ncn(-c3ccccc3)c2N4c2ccc(C(C)(C)C)cc2)cc1 ; c1ccc(-c2ccc(N3c4cccc5c4B(c4ncn(-c6ccccc6)c43)c3ncn(-c4ccccc4)c3N5c3ccc(-c4ccccc4)cc3)cc2)cc1 ; CC(C)(C)c1cc2c3c(c1)N(c1ccccc1)c1c(ncn1-c1ccccc1)B3c1ncn(-c3ccccc3)c1N2c1ccccc1 ; c1ccc(-c2cc3c4c(c2)N(c2ccccc2)c2c(ncn2-c2ccccc2)B4c2ncn(-c4ccccc4)c2N3c2ccccc2)cc1]

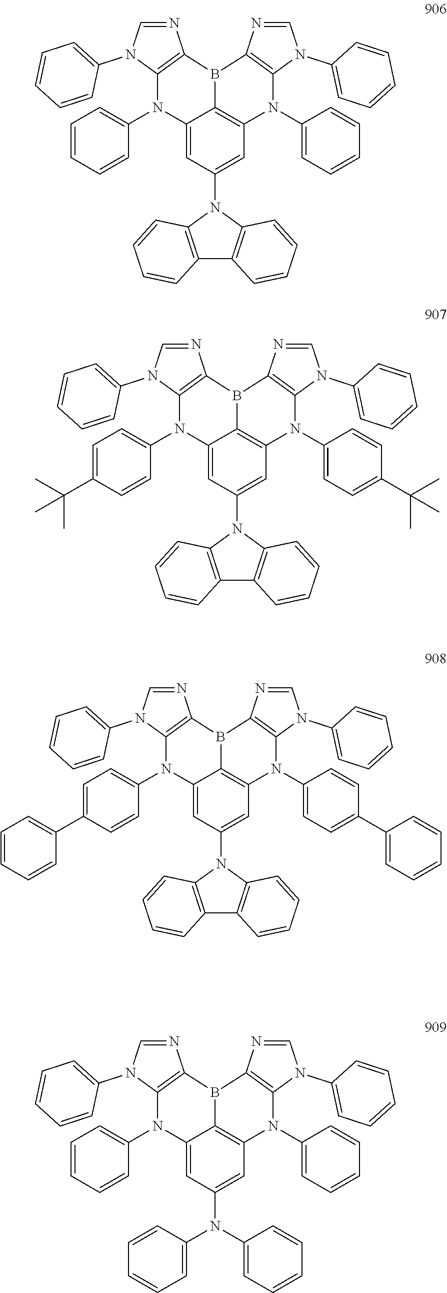 [SMILES: c1ccc(N2c3cc(-n4c5ccccc5c5ccccc54)cc4c3B(c3ncn(-c5ccccc5)c32)c2ncn(-c3ccccc3)c2N4c2ccccc2)cc1 ; CC(C)(C)c1ccc(N2c3cc(-n4c5ccccc5c5ccccc54)cc4c3B(c3ncn(-c5ccccc5)c32)c2ncn(-c3ccccc3)c2N4c2ccc(C(C)(C)C)cc2)cc1 ; c1ccc(-c2ccc(N3c4cc(-n5c6ccccc6c6ccccc65)cc5c4B(c4ncn(-c6ccccc6)c43)c3ncn(-c4ccccc4)c3N5c3ccc(-c4ccccc4)cc3)cc2)cc1 ; c1ccc(N(c2ccccc2)c2cc3c4c(c2)N(c2ccccc2)c2c(ncn2-c2ccccc2)B4c2ncn(-c4ccccc4)c2N3c2ccccc2)cc1]

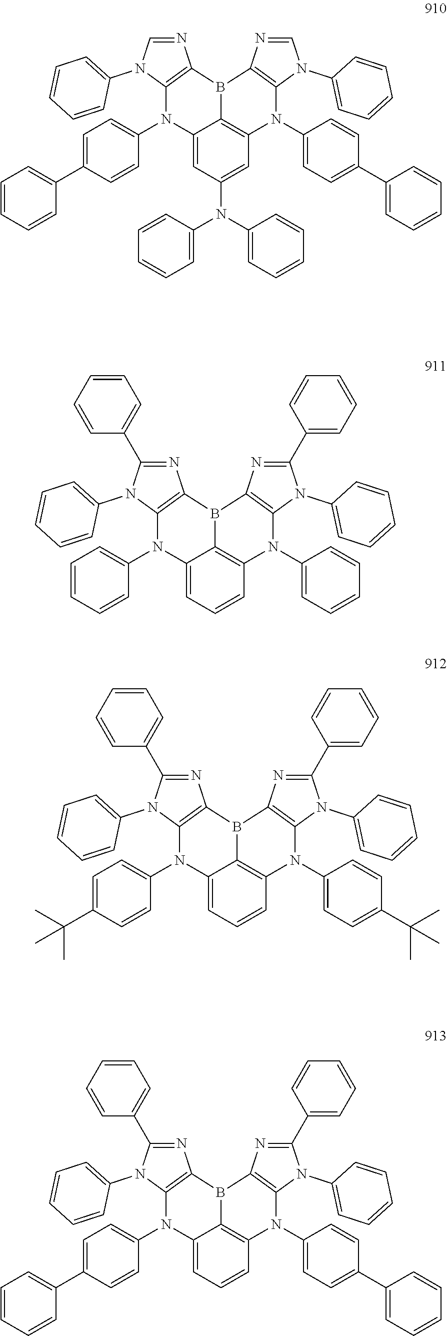 [SMILES: c1ccc(-c2ccc(N3c4cc(N(c5ccccc5)c5ccccc5)cc5c4B(c4ncn(-c6ccccc6)c43)c3ncn(-c4ccccc4)c3N5c3ccc(-c4ccccc4)cc3)cc2)cc1 ; c1ccc(-c2nc3c(n2-c2ccccc2)N(c2ccccc2)c2cccc4c2B3c2nc(-c3ccccc3)n(-c3ccccc3)c2N4c2ccccc2)cc1 ; CC(C)(C)c1ccc(N2c3cccc4c3B(c3nc(-c5ccccc5)n(-c5ccccc5)c32)c2nc(-c3ccccc3)n(-c3ccccc3)c2N4c2ccc(C(C)(C)C)cc2)cc1 ; c1ccc(-c2ccc(N3c4cccc5c4B(c4nc(-c6ccccc6)n(-c6ccccc6)c43)c3nc(-c4ccccc4)n(-c4ccccc4)c3N5c3ccc(-c4ccccc4)cc3)cc2)cc1]

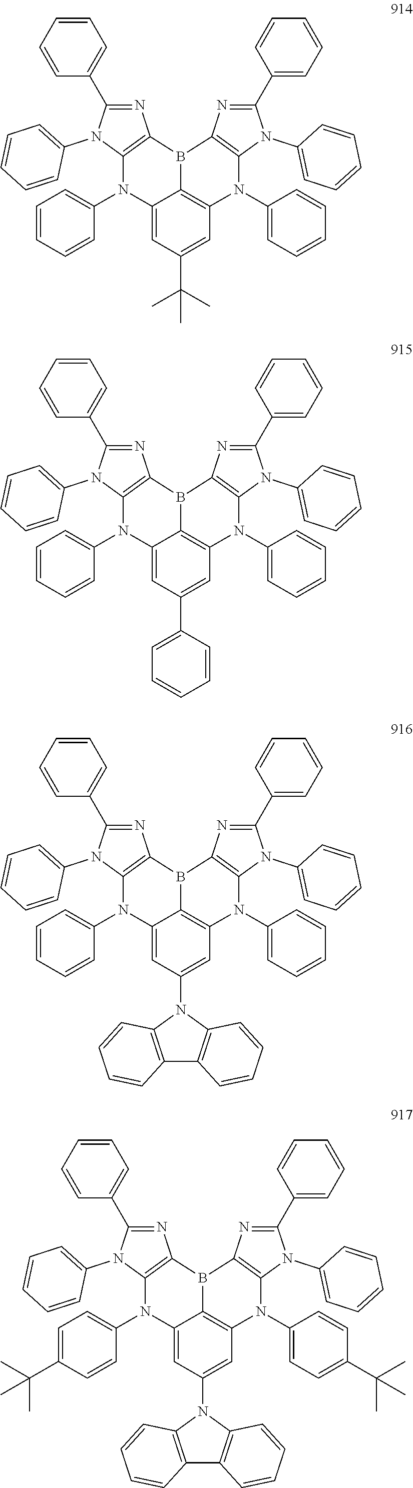 [SMILES: CC(C)(C)c1cc2c3c(c1)N(c1ccccc1)c1c(nc(-c4ccccc4)n1-c1ccccc1)B3c1nc(-c3ccccc3)n(-c3ccccc3)c1N2c1ccccc1 ; c1ccc(-c2cc3c4c(c2)N(c2ccccc2)c2c(nc(-c5ccccc5)n2-c2ccccc2)B4c2nc(-c4ccccc4)n(-c4ccccc4)c2N3c2ccccc2)cc1 ; c1ccc(-c2nc3c(n2-c2ccccc2)N(c2ccccc2)c2cc(-n4c5ccccc5c5ccccc54)cc4c2B3c2nc(-c3ccccc3)n(-c3ccccc3)c2N4c2ccccc2)cc1 ; CC(C)(C)c1ccc(N2c3cc(-n4c5ccccc5c5ccccc54)cc4c3B(c3nc(-c5ccccc5)n(-c5ccccc5)c32)c2nc(-c3ccccc3)n(-c3ccccc3)c2N4c2ccc(C(C)(C)C)cc2)cc1]

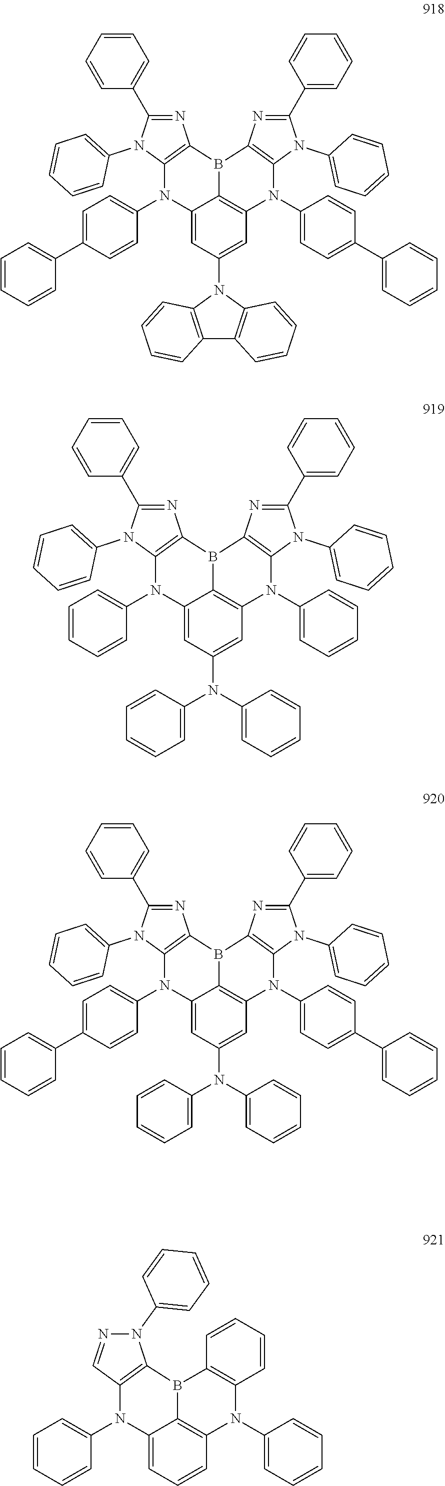 [SMILES: c1ccc(-c2ccc(N3c4cc(-n5c6ccccc6c6ccccc65)cc5c4B(c4nc(-c6ccccc6)n(-c6ccccc6)c43)c3nc(-c4ccccc4)n(-c4ccccc4)c3N5c3ccc(-c4ccccc4)cc3)cc2)cc1 ; c1ccc(-c2nc3c(n2-c2ccccc2)N(c2ccccc2)c2cc(N(c4ccccc4)c4ccccc4)cc4c2B3c2nc(-c3ccccc3)n(-c3ccccc3)c2N4c2ccccc2)cc1 ; c1ccc(-c2ccc(N3c4cc(N(c5ccccc5)c5ccccc5)cc5c4B(c4nc(-c6ccccc6)n(-c6ccccc6)c43)c3nc(-c4ccccc4)n(-c4ccccc4)c3N5c3ccc(-c4ccccc4)cc3)cc2)cc1 ; c1ccc(N2c3ccccc3B3c4c2cccc4N(c2ccccc2)c2cnn(-c4ccccc4)c23)cc1]

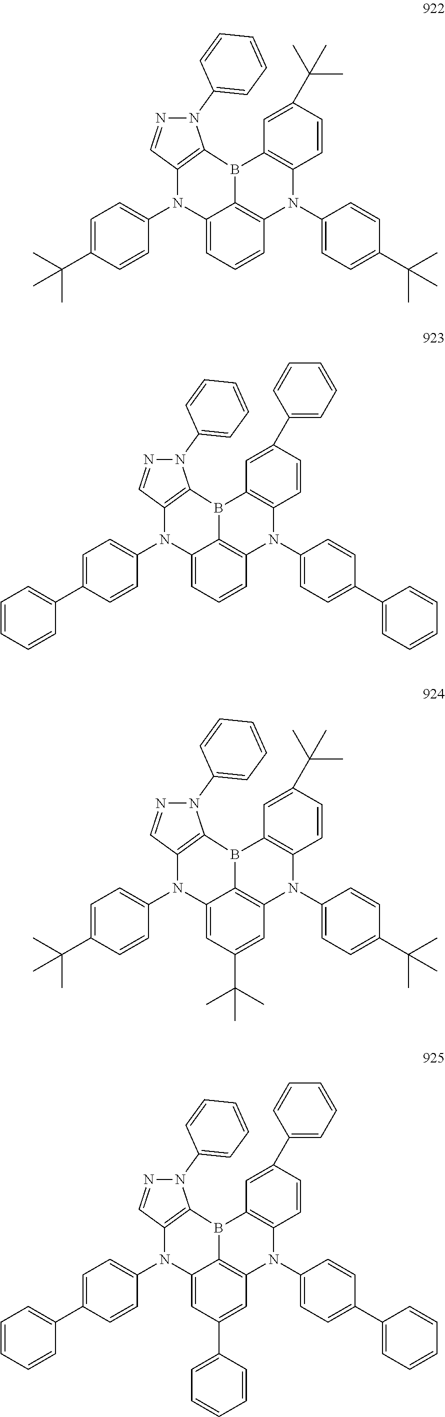

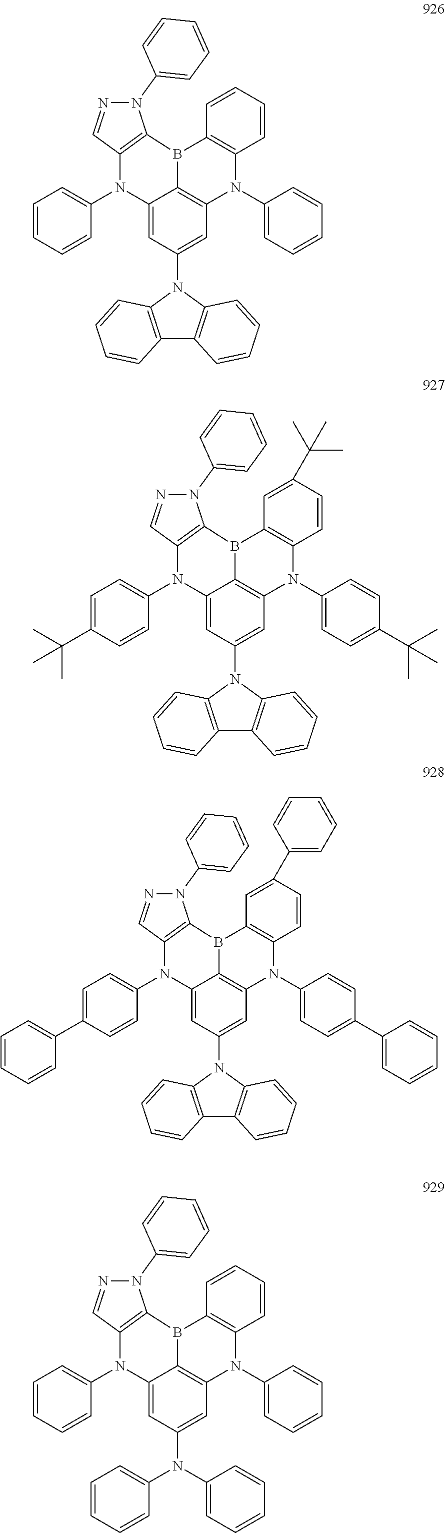

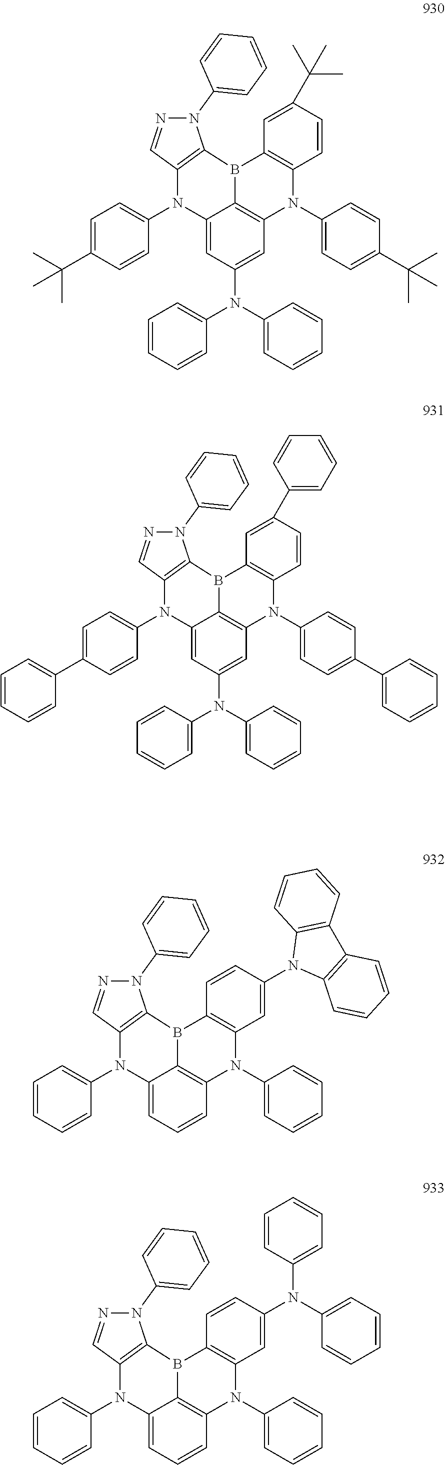 [SMILES: CC(C)(C)c1ccc(N2c3ccc(C(C)(C)C)cc3B3c4c2cc(N(c2ccccc2)c2ccccc2)cc4N(c2ccc(C(C)(C)C)cc2)c2cnn(-c4ccccc4)c23)cc1 ; c1ccc(-c2ccc(N3c4ccc(-c5ccccc5)cc4B4c5c3cc(N(c3ccccc3)c3ccccc3)cc5N(c3ccc(-c5ccccc5)cc3)c3cnn(-c5ccccc5)c34)cc2)cc1 ; c1ccc(N2c3cc(-n4c5ccccc5c5ccccc54)ccc3B3c4c2cccc4N(c2ccccc2)c2cnn(-c4ccccc4)c23)cc1 ; c1ccc(N(c2ccccc2)c2ccc3c(c2)N(c2ccccc2)c2cccc4c2B3c2c(cnn2-c2ccccc2)N4c2ccccc2)cc1]

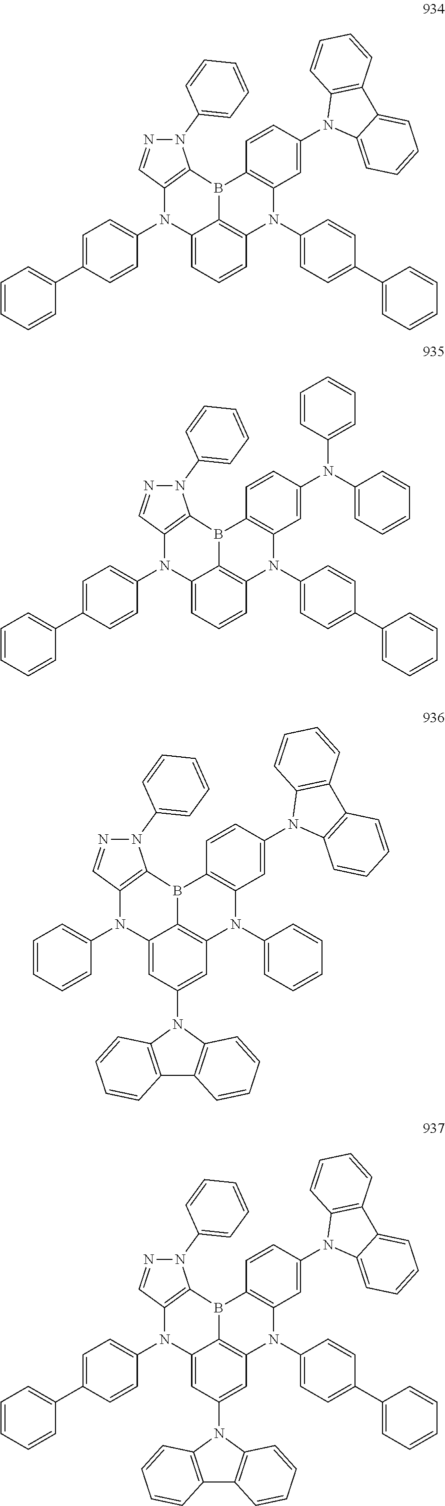 [SMILES: c1ccc(-c2ccc(N3c4cc(-n5c6ccccc6c6ccccc65)ccc4B4c5c3cccc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3cnn(-c5ccccc5)c34)cc2)cc1 ; c1ccc(N2c3cc(-n4c5ccccc5c5ccccc54)ccc3B3c4c2cc(-n2c5ccccc5c5ccccc52)cc4N(c2ccccc2)c2cnn(-c4ccccc4)c23)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3cnn(-c5ccccc5)c34)cc2)cc1]

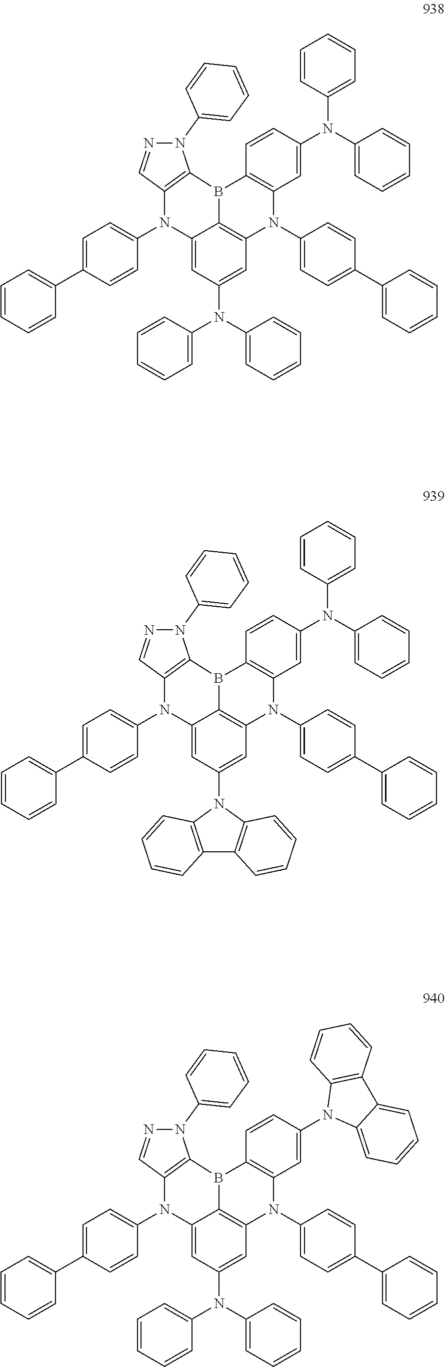 [SMILES: c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3cnn(-c5ccccc5)c34)cc2)cc1 ; c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3cnn(-c5ccccc5)c34)cc2)cc1]

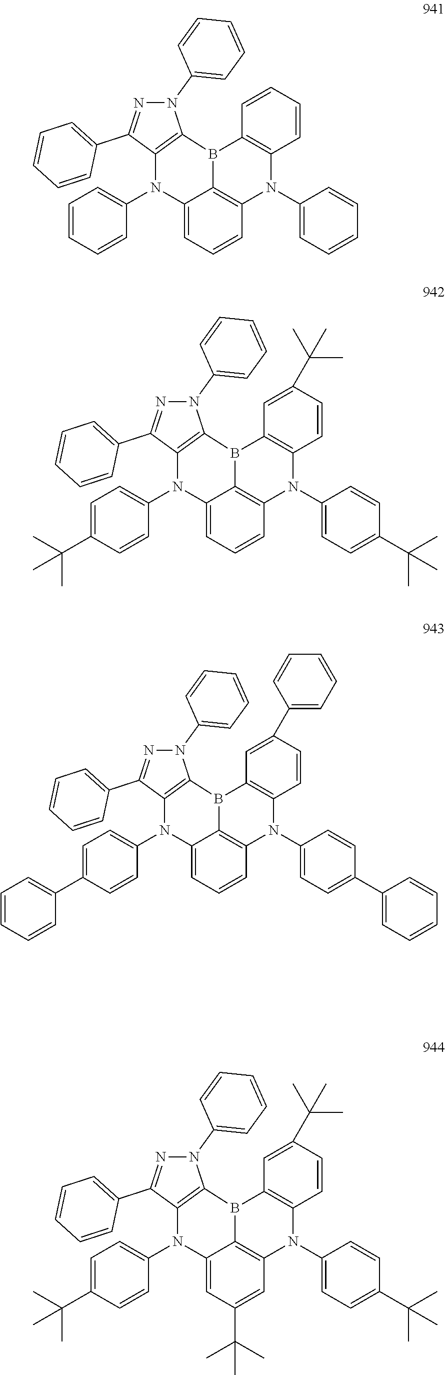

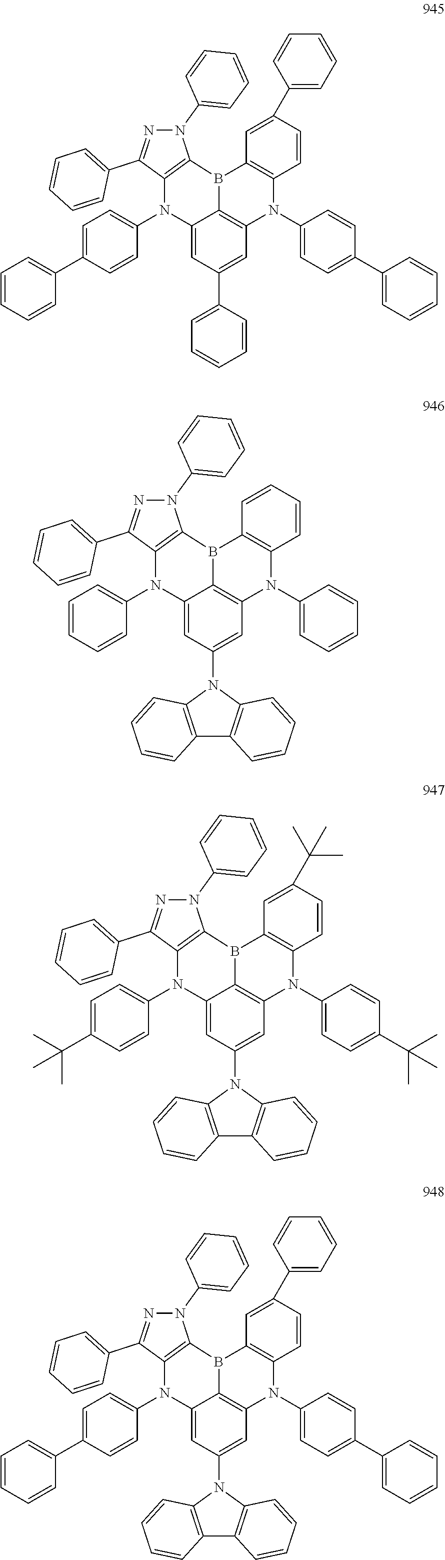 [SMILES: c1ccc(-c2ccc(N3c4ccc(-c5ccccc5)cc4B4c5c3cc(-c3ccccc3)cc5N(c3ccc(-c5ccccc5)cc3)c3c(-c5ccccc5)nn(-c5ccccc5)c34)cc2)cc1 ; c1ccc(-c2nn(-c3ccccc3)c3c2N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2c(-c4ccccc4)nn(-c4ccccc4)c23)cc1 ; c1ccc(-c2ccc(N3c4ccc(-c5ccccc5)cc4B4c5c3cc(-n3c6ccccc6c6ccccc63)cc5N(c3ccc(-c5ccccc5)cc3)c3c(-c5ccccc5)nn(-c5ccccc5)c34)cc2)cc1]

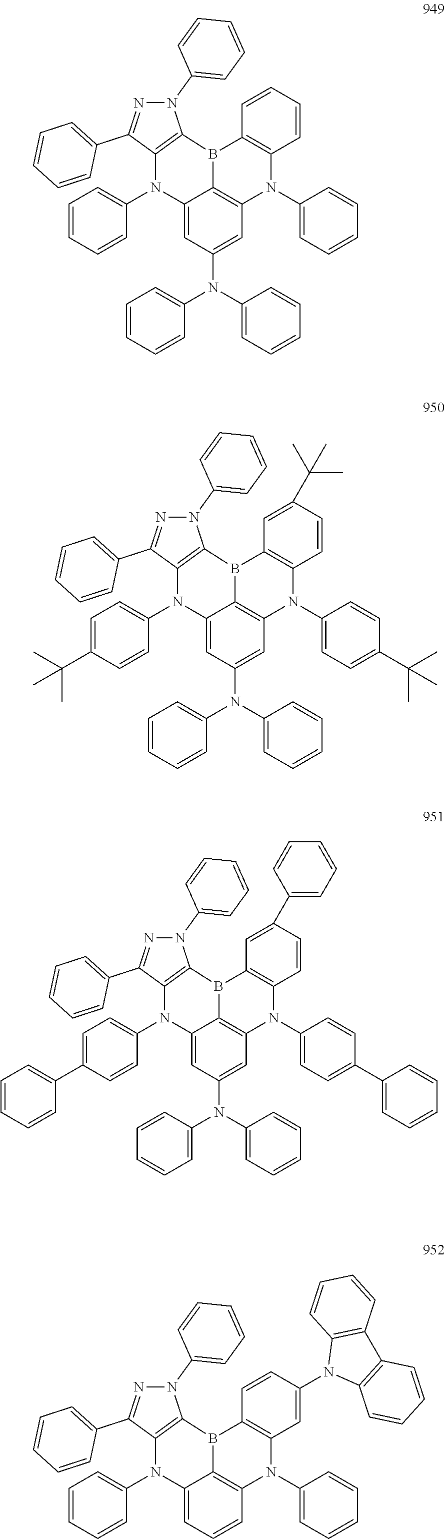 [SMILES: c1ccc(-c2nn(-c3ccccc3)c3c2N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4c2cc(N(c2ccccc2)c2ccccc2)cc4N(c2ccc(C(C)(C)C)cc2)c2c(-c4ccccc4)nn(-c4ccccc4)c23)cc1 ; c1ccc(-c2ccc(N3c4ccc(-c5ccccc5)cc4B4c5c3cc(N(c3ccccc3)c3ccccc3)cc5N(c3ccc(-c5ccccc5)cc3)c3c(-c5ccccc5)nn(-c5ccccc5)c34)cc2)cc1 ; c1ccc(-c2nn(-c3ccccc3)c3c2N(c2ccccc2)c2cccc4c2B3c2ccc(-n3c5ccccc5c5ccccc53)cc2N4c2ccccc2)cc1]

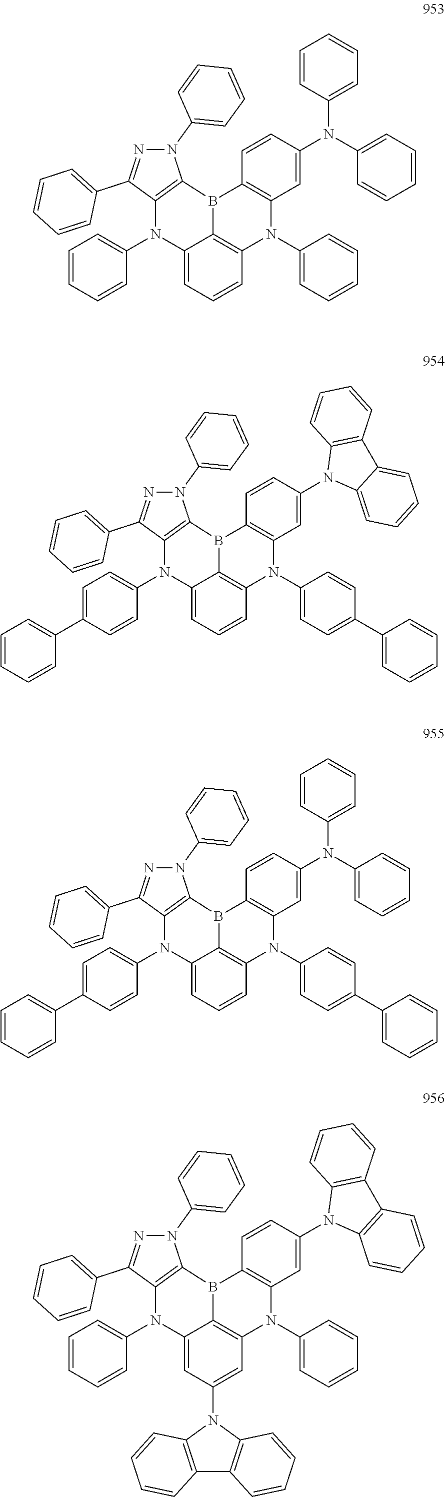 [SMILES: c1ccc(-c2nn(-c3ccccc3)c3c2N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5c3cccc5N(c3ccc(-c5ccccc5)cc3)c3c(-c5ccccc5)nn(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3c(-c5ccccc5)nn(-c5ccccc5)c34)cc2)cc1 ; c1ccc(-c2nn(-c3ccccc3)c3c2N(c2ccccc2)c2cc(-n4c5ccccc5c5ccccc54)cc4c2B3c2ccc(-n3c5ccccc5c5ccccc53)cc2N4c2ccccc2)cc1]

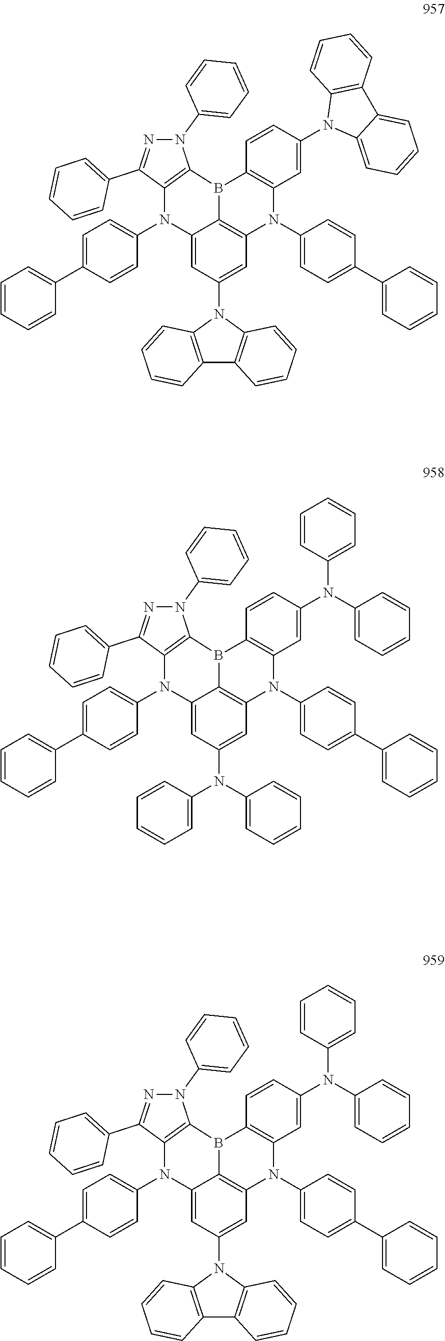 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3c(-c5ccccc5)nn(-c5ccccc5)c34)cc2)cc1 ; c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3c(-c5ccccc5)nn(-c5ccccc5)c34)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3c(-c5ccccc5)nn(-c5ccccc5)c34)cc2)cc1]

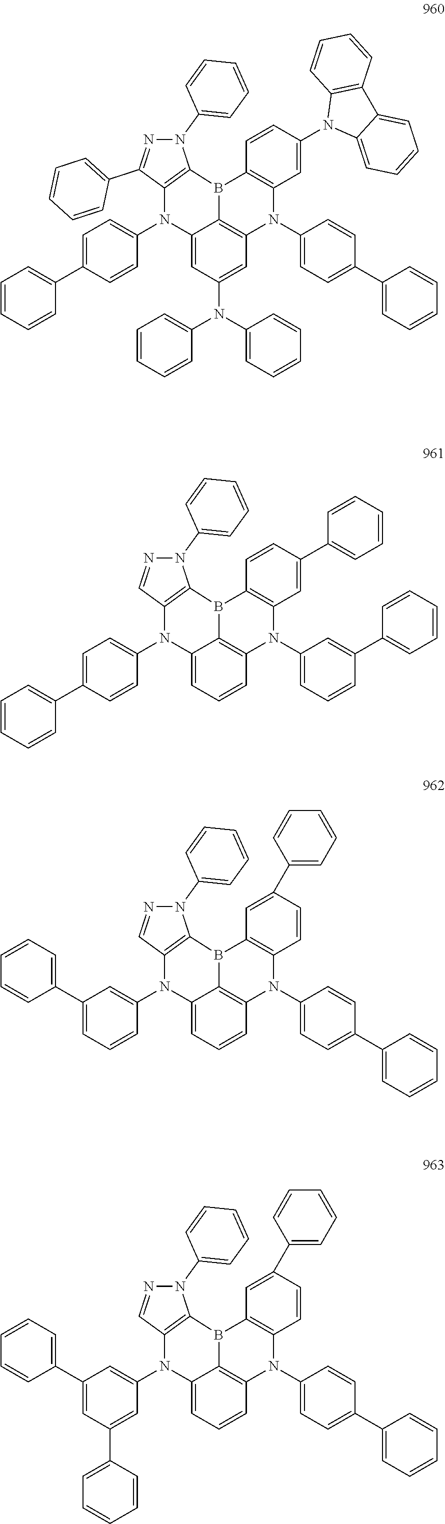 [SMILES: c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3c(-c5ccccc5)nn(-c5ccccc5)c34)cc2)cc1 ; c1ccc(-c2ccc(N3c4cccc5c4B(c4ccc(-c6ccccc6)cc4N5c4cccc(-c5ccccc5)c4)c4c3cnn4-c3ccccc3)cc2)cc1 ; c1ccc(-c2ccc(N3c4ccc(-c5ccccc5)cc4B4c5c3cccc5N(c3cccc(-c5ccccc5)c3)c3cnn(-c5ccccc5)c34)cc2)cc1 ; c1ccc(-c2ccc(N3c4ccc(-c5ccccc5)cc4B4c5c3cccc5N(c3cc(-c5ccccc5)cc(-c5ccccc5)c3)c3cnn(-c5ccccc5)c34)cc2)cc1]

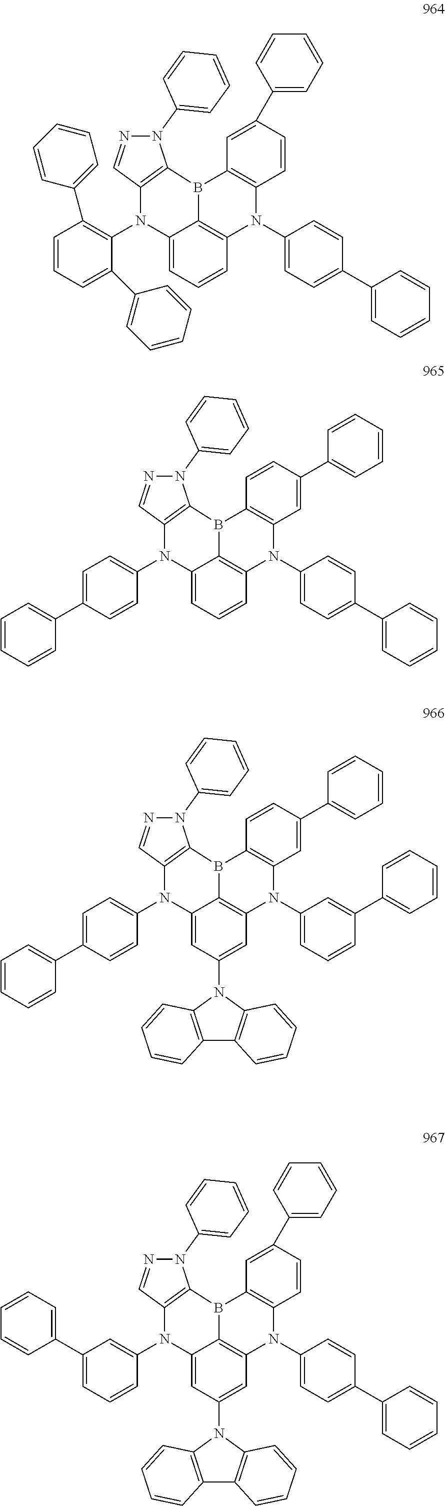 [SMILES: c1ccc(-c2ccc(N3c4ccc(-c5ccccc5)cc4B4c5c3cccc5N(c3c(-c5ccccc5)cccc3-c3ccccc3)c3cnn(-c5ccccc5)c34)cc2)cc1 ; c1ccc(-c2ccc(N3c4cc(-c5ccccc5)ccc4B4c5c3cccc5N(c3ccc(-c5ccccc5)cc3)c3cnn(-c5ccccc5)c34)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c3cnn4-c3ccccc3)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3cnn(-c5ccccc5)c34)cc2)cc1]

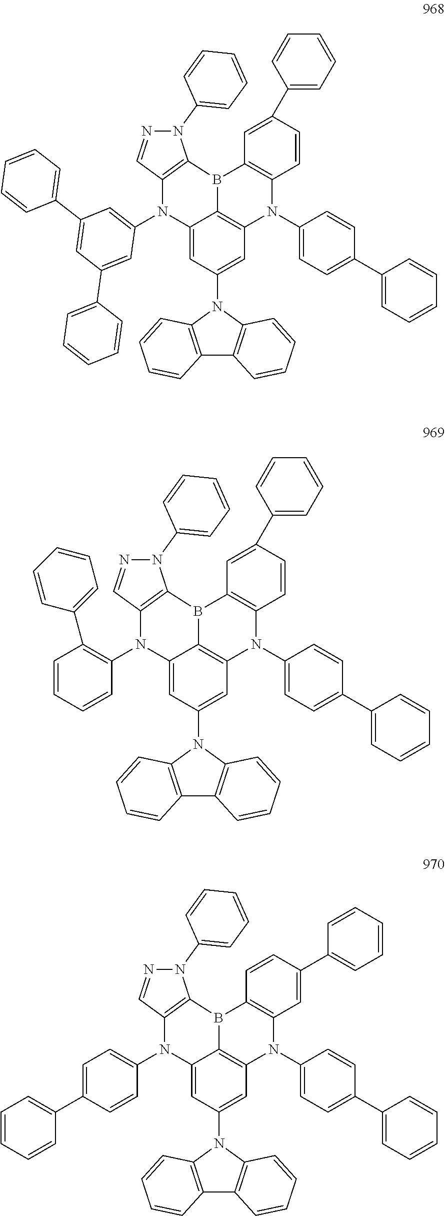 [SMILES: c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3cnn(-c5ccccc5)c34)cc2)cc1 ; c1ccc(-c2ccc(N3c4ccc(-c5ccccc5)cc4B4c5c3cc(-n3c6ccccc6c6ccccc63)cc5N(c3ccccc3-c3ccccc3)c3cnn(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3cnn(-c5ccccc5)c34)cc2)cc1]

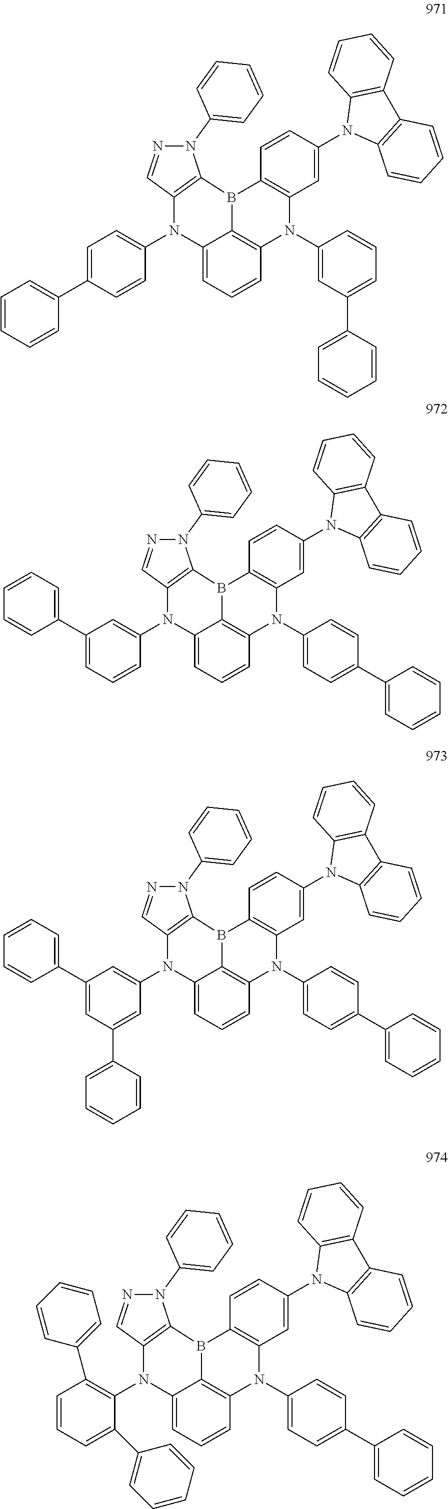

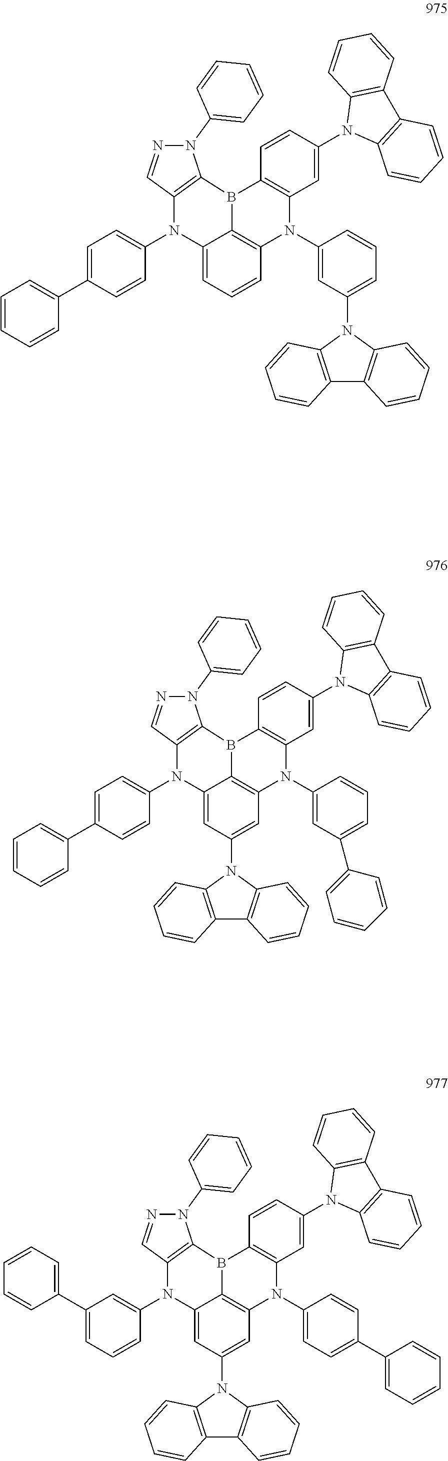 [SMILES: c1ccc(-c2ccc(N3c4cccc5c4B(c4ccc(-n6c7ccccc7c7ccccc76)cc4N5c4cccc(-n5c6ccccc6c6ccccc65)c4)c4c3cnn4-c3ccccc3)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3cnn4-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3cnn(-c5ccccc5)c34)cc2)cc1]

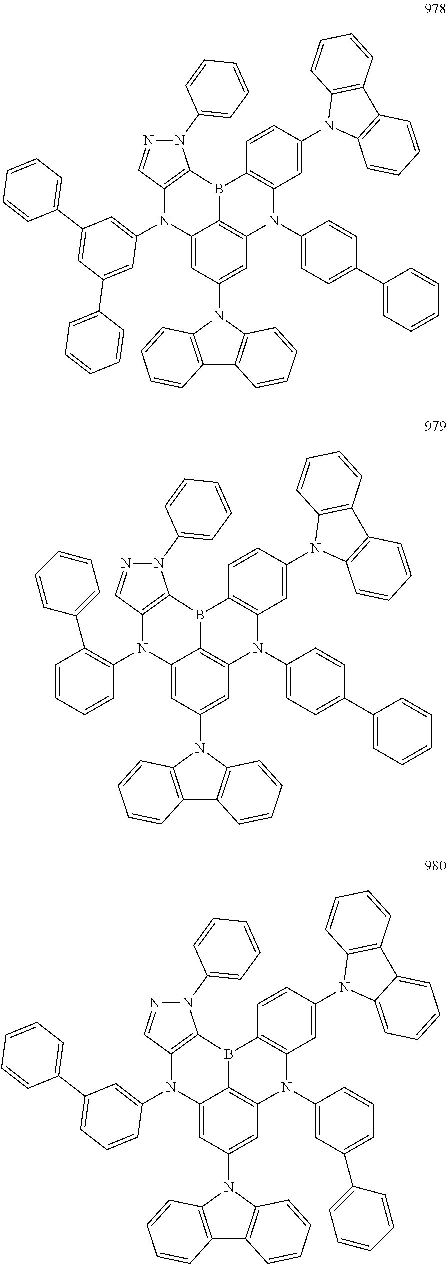 [SMILES: c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3cnn(-c5ccccc5)c34)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3cnn(-c5ccccc5)c34)cc2)cc1 ; c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3cnn(-c5ccccc5)c34)c2)cc1]

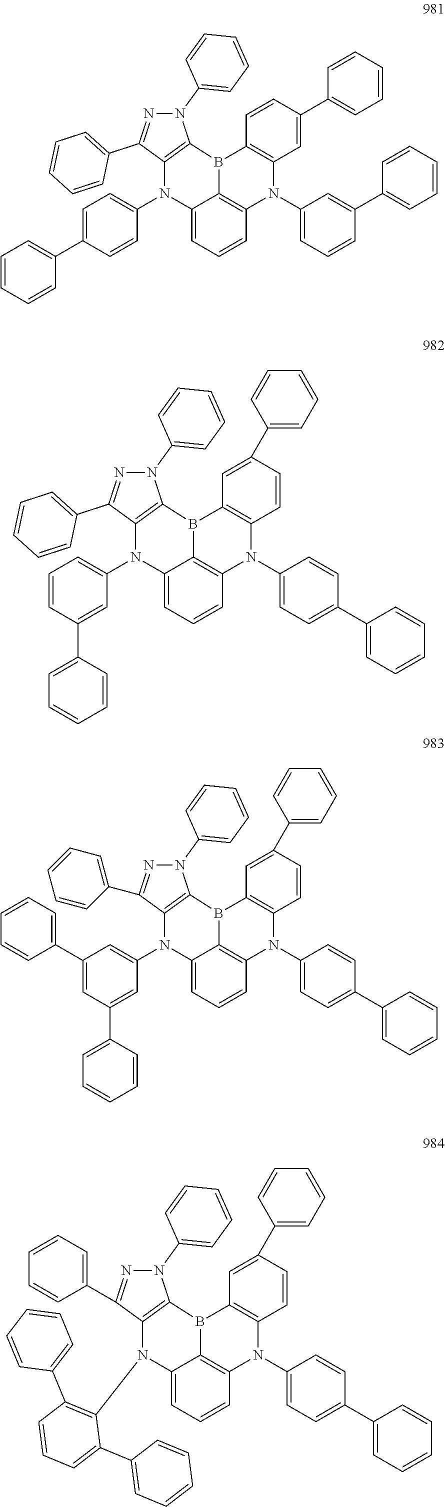 [SMILES: c1ccc(-c2ccc(N3c4cccc5c4B(c4ccc(-c6ccccc6)cc4N5c4cccc(-c5ccccc5)c4)c4c3c(-c3ccccc3)nn4-c3ccccc3)cc2)cc1 ; c1ccc(-c2ccc(N3c4ccc(-c5ccccc5)cc4B4c5c3cccc5N(c3cccc(-c5ccccc5)c3)c3c(-c5ccccc5)nn(-c5ccccc5)c34)cc2)cc1 ; c1ccc(-c2ccc(N3c4ccc(-c5ccccc5)cc4B4c5c3cccc5N(c3cc(-c5ccccc5)cc(-c5ccccc5)c3)c3c(-c5ccccc5)nn(-c5ccccc5)c34)cc2)cc1 ; c1ccc(-c2ccc(N3c4ccc(-c5ccccc5)cc4B4c5c3cccc5N(c3c(-c5ccccc5)cccc3-c3ccccc3)c3c(-c5ccccc5)nn(-c5ccccc5)c34)cc2)cc1]

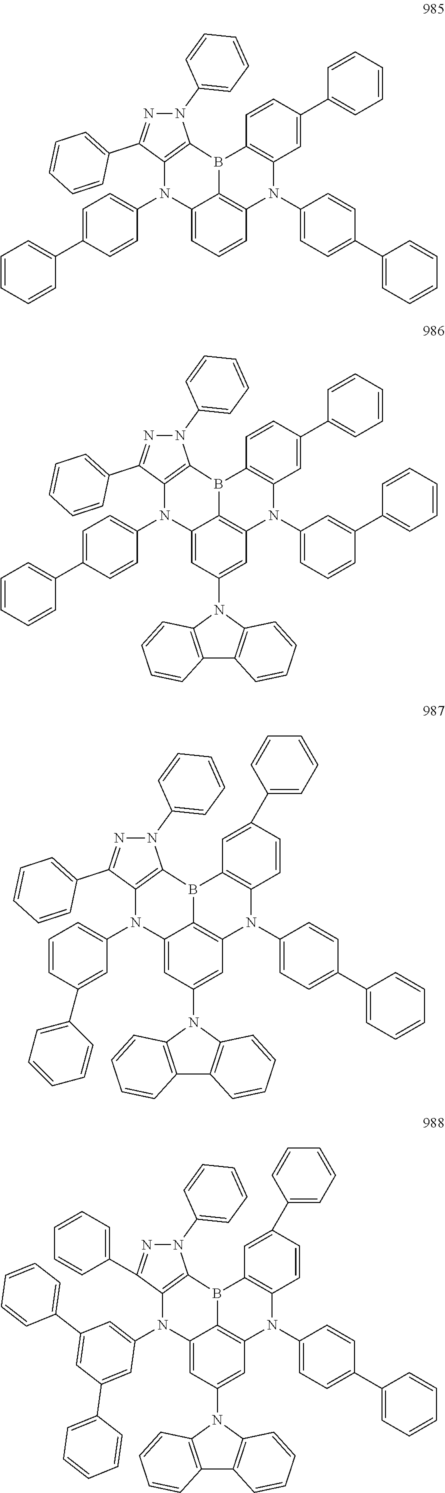

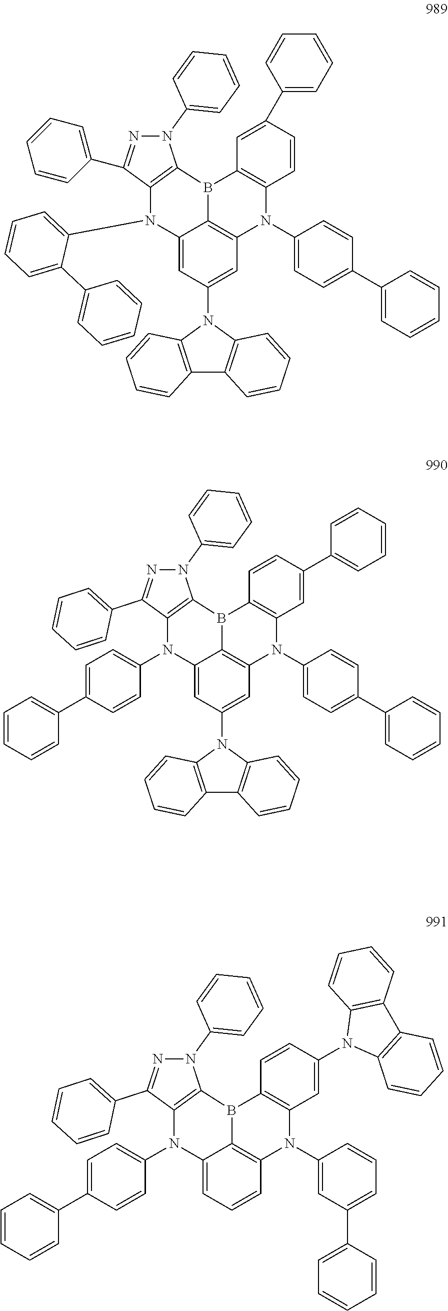 [SMILES: c1ccc(-c2ccc(N3c4ccc(-c5ccccc5)cc4B4c5c3cc(-n3c6ccccc6c6ccccc63)cc5N(c3ccccc3-c3ccccc3)c3c(-c5ccccc5)nn(-c5ccccc5)c34)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3c(-c5ccccc5)nn(-c5ccccc5)c34)cc2)cc1 ; c1ccc(-c2ccc(N3c4cccc5c4B(c4ccc(-n6c7ccccc7c7ccccc76)cc4N5c4cccc(-c5ccccc5)c4)c4c3c(-c3ccccc3)nn4-c3ccccc3)cc2)cc1]

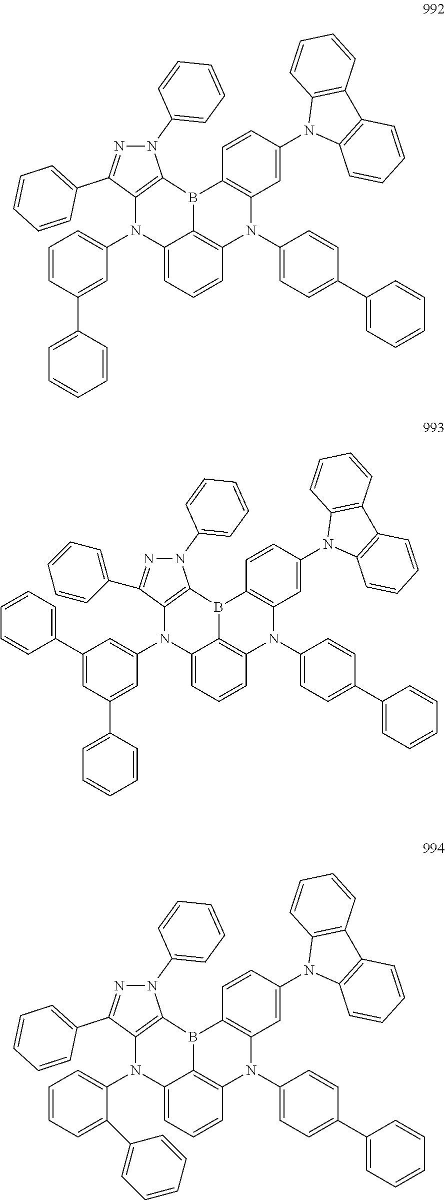 [SMILES: c1ccc(-c2ccc(N3c4cc(-n5c6ccccc6c6ccccc65)ccc4B4c5c3cccc5N(c3cccc(-c5ccccc5)c3)c3c(-c5ccccc5)nn(-c5ccccc5)c34)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3c(-c5ccccc5)nn(-c5ccccc5)c34)cc2)cc1 ; c1ccc(-c2ccc(N3c4cc(-n5c6ccccc6c6ccccc65)ccc4B4c5c3cccc5N(c3ccccc3-c3ccccc3)c3c(-c5ccccc5)nn(-c5ccccc5)c34)cc2)cc1]

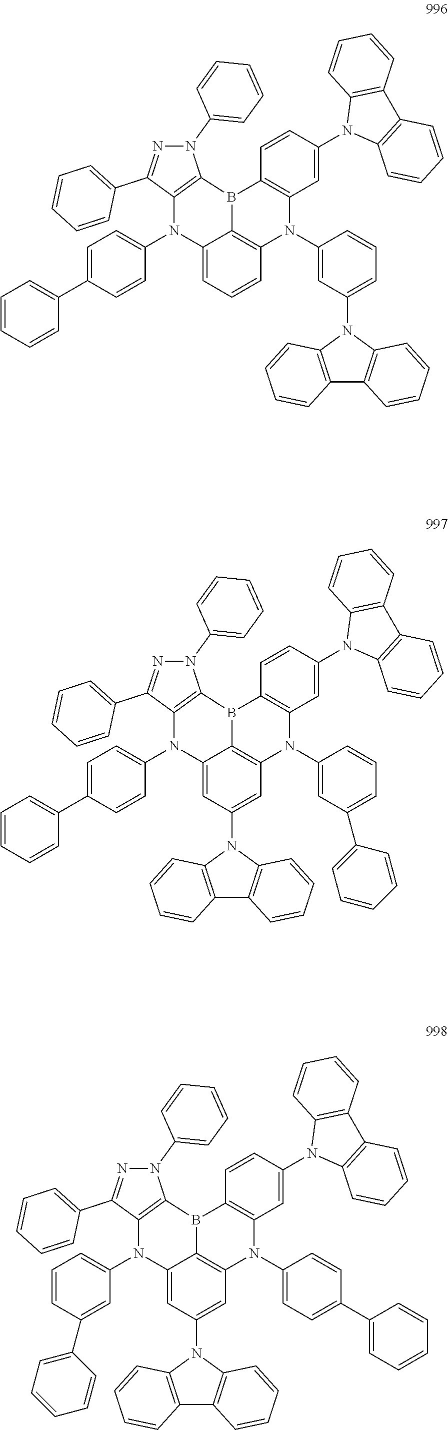 [SMILES: c1ccc(-c2ccc(N3c4cccc5c4B(c4ccc(-n6c7ccccc7c7ccccc76)cc4N5c4cccc(-n5c6ccccc6c6ccccc65)c4)c4c3c(-c3ccccc3)nn4-c3ccccc3)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c3c(-c3ccccc3)nn4-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3c(-c5ccccc5)nn(-c5ccccc5)c34)cc2)cc1]

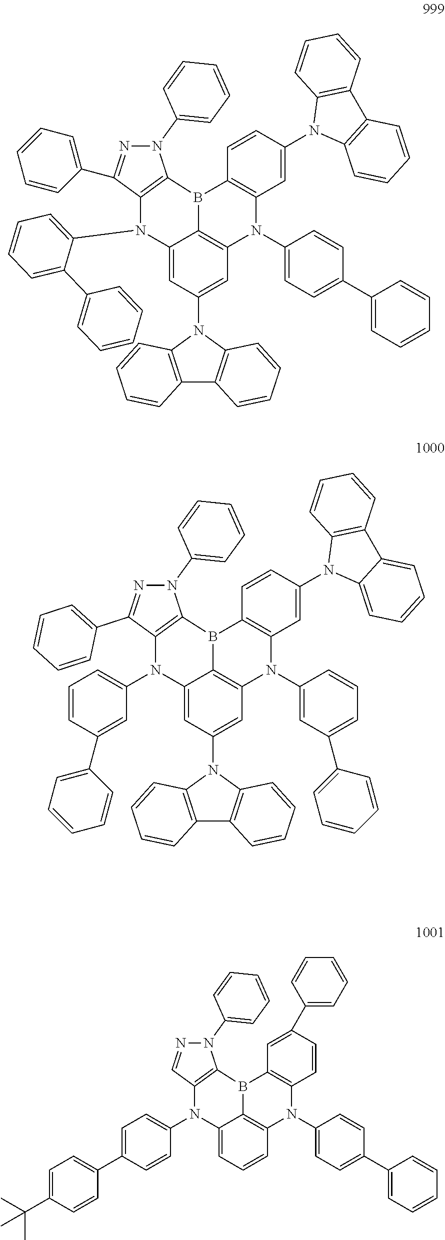 [SMILES: c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3c(-c5ccccc5)nn(-c5ccccc5)c34)cc2)cc1 ; c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3c(-c5ccccc5)nn(-c5ccccc5)c34)c2)cc1 ; CC(C)(C)c1ccc(-c2ccc(N3c4cccc5c4B(c4cc(-c6ccccc6)ccc4N5c4ccc(-c5ccccc5)cc4)c4c3cnn4-c3ccccc3)cc2)cc1]

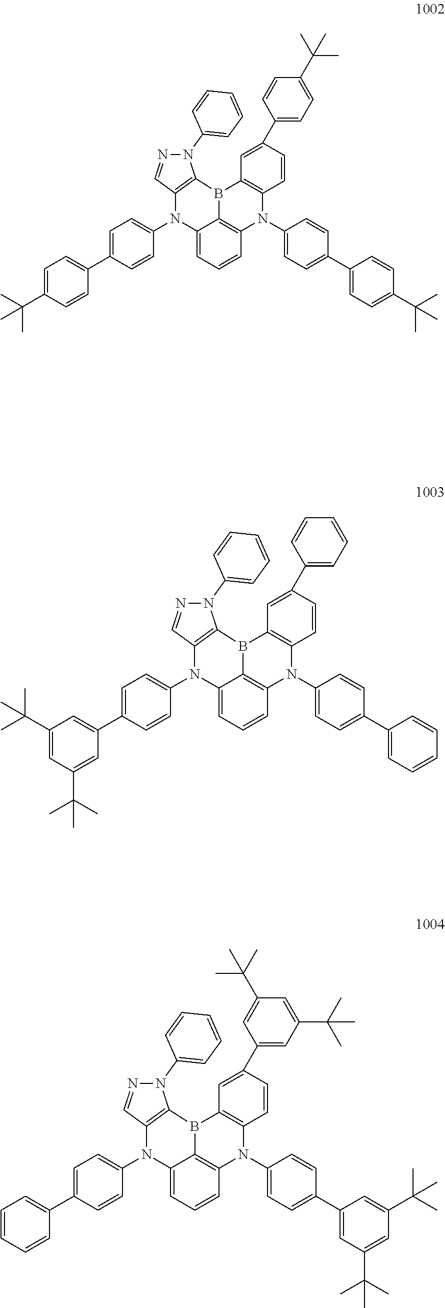

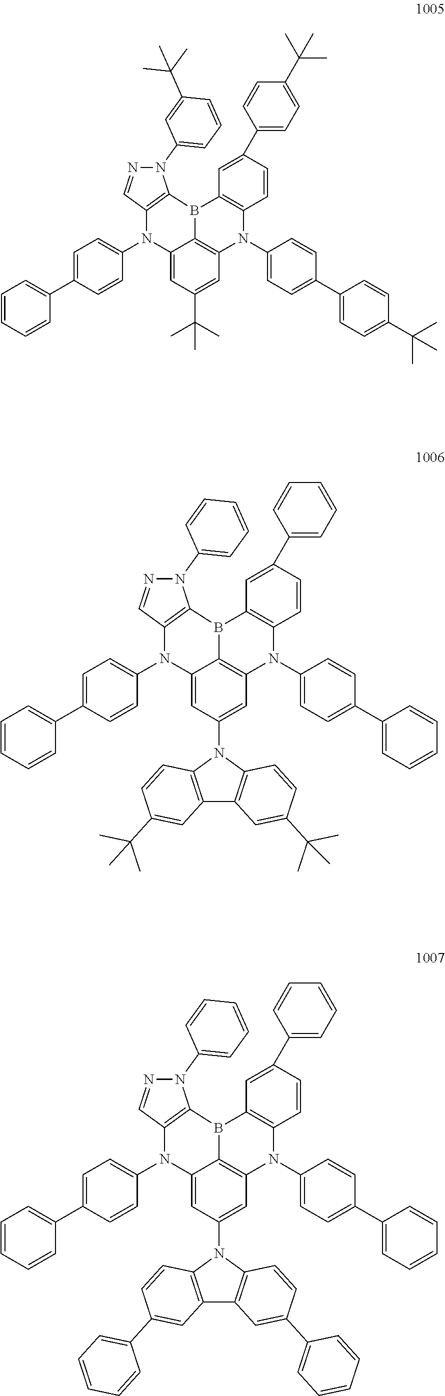 [SMILES: CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3cnn(-c5cccc(C(C)(C)C)c5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1cnn(-c4ccccc4)c1B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3cnn(-c5ccccc5)c34)cc2)cc1]

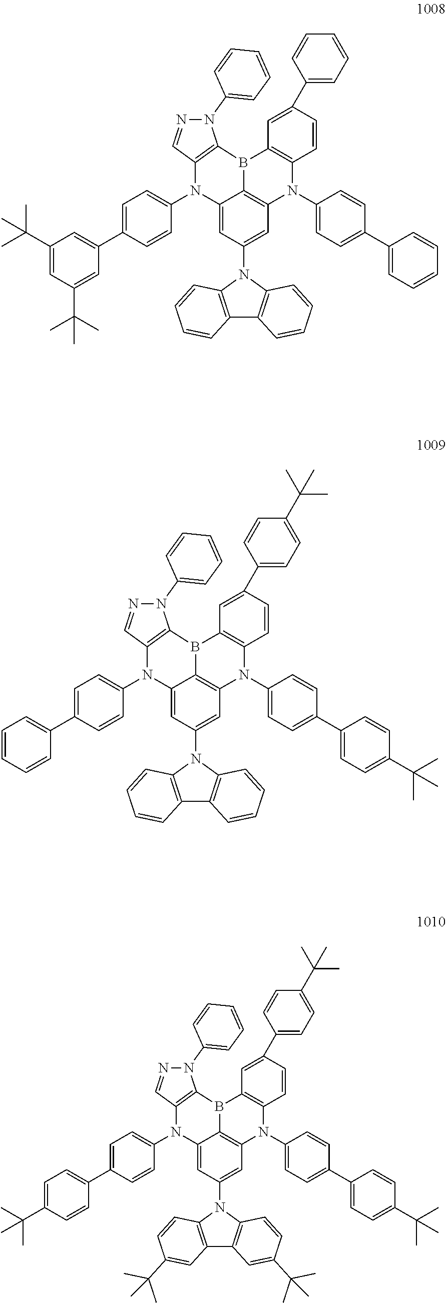

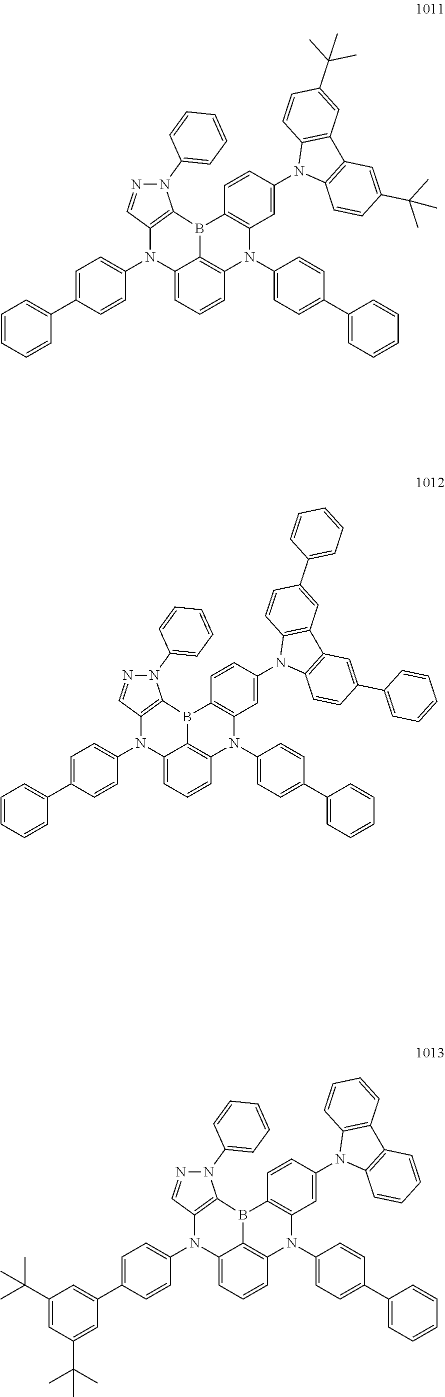

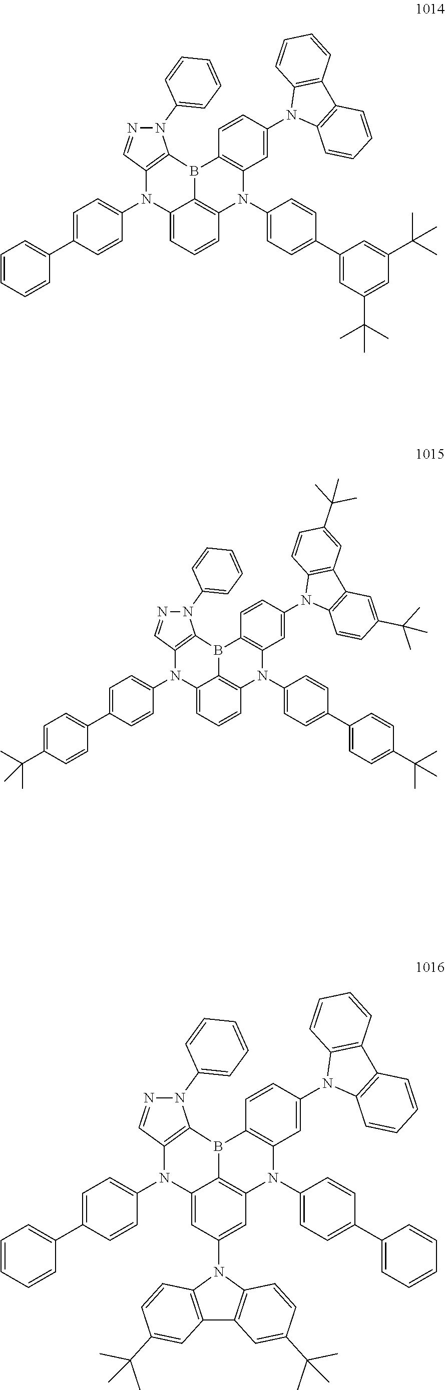

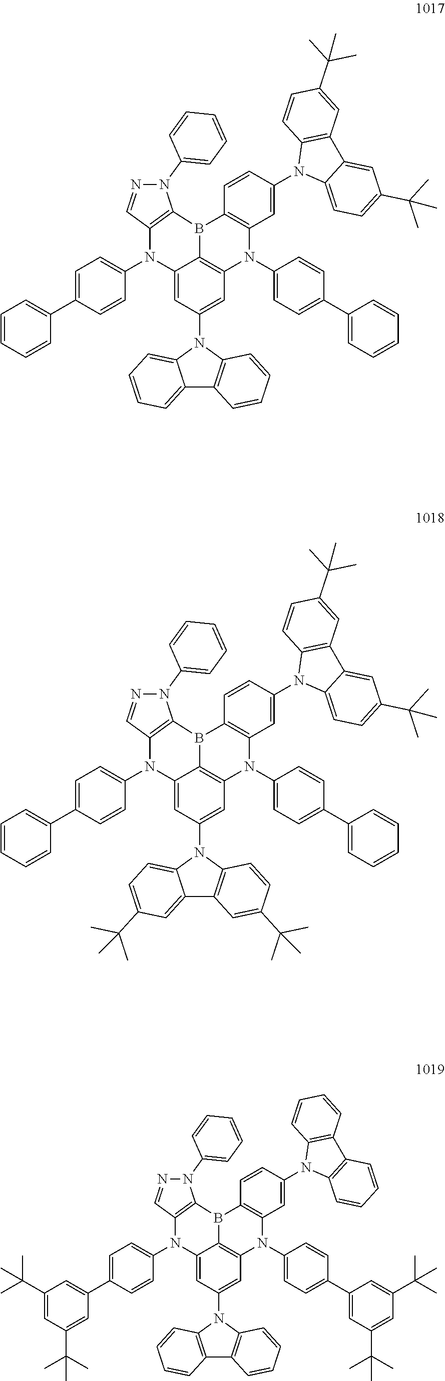

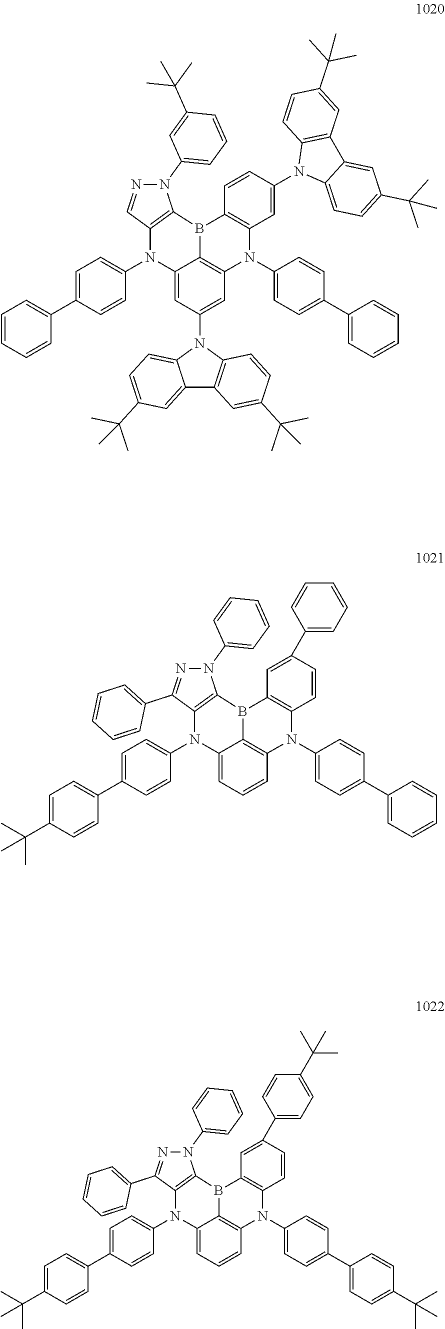

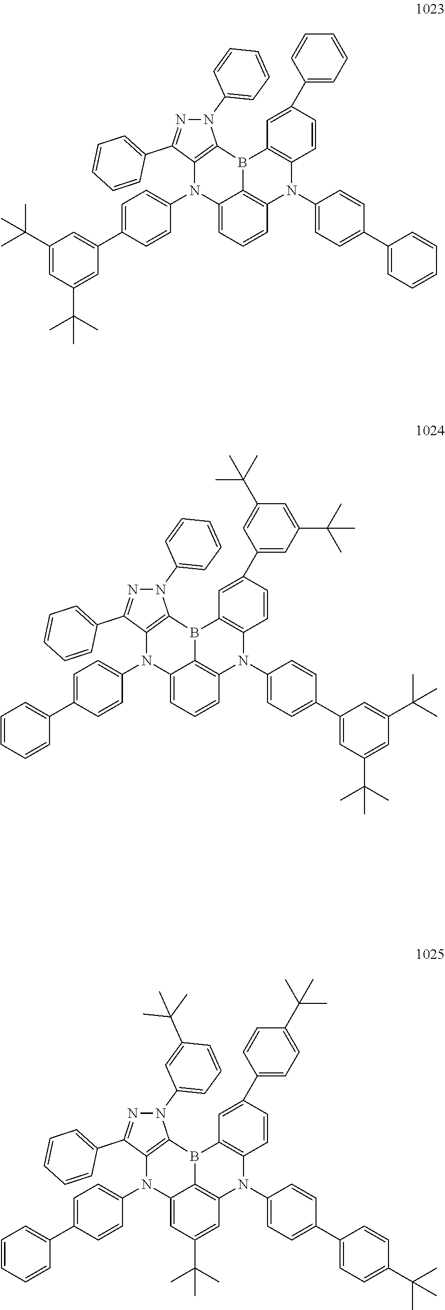 [SMILES: CC(C)(C)c1cc(-c2ccc(N3c4cccc5c4B(c4cc(-c6ccccc6)ccc4N5c4ccc(-c5ccccc5)cc4)c4c3c(-c3ccccc3)nn4-c3ccccc3)cc2)cc(C(C)(C)C)c1 ; CC(C)(C)c1cc(-c2ccc(N3c4ccc(-c5cc(C(C)(C)C)cc(C(C)(C)C)c5)cc4B4c5c3cccc5N(c3ccc(-c5ccccc5)cc3)c3c(-c5ccccc5)nn(-c5ccccc5)c34)cc2)cc(C(C)(C)C)c1 ; CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3c(-c5ccccc5)nn(-c5cccc(C(C)(C)C)c5)c34)cc2)cc1]

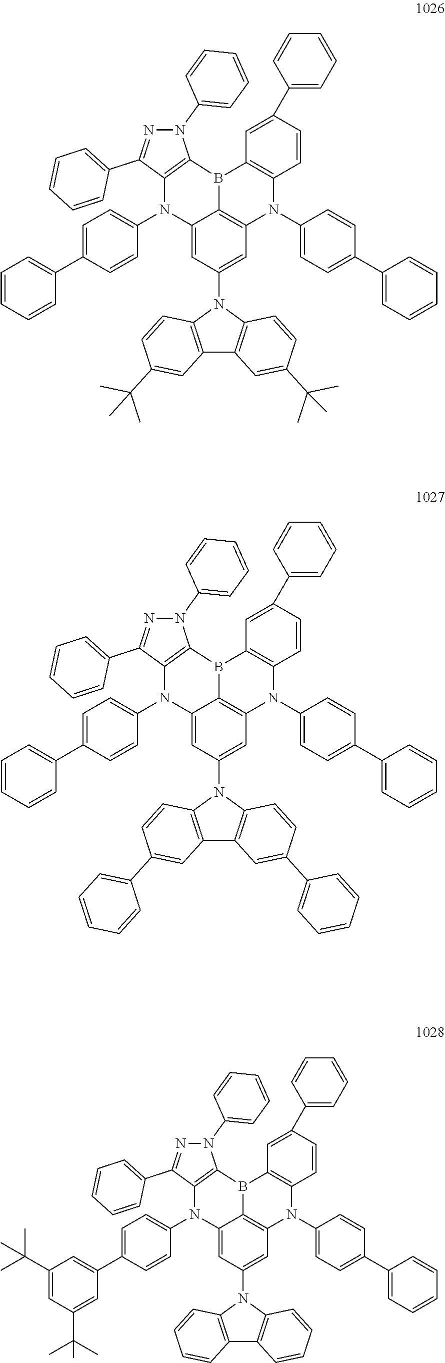 [SMILES: CC(C)(C)c1ccc2c(c1)c1cc(C(C)(C)C)ccc1n2-c1cc2c3c(c1)N(c1ccc(-c4ccccc4)cc1)c1c(-c4ccccc4)nn(-c4ccccc4)c1B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3c(-c5ccccc5)nn(-c5ccccc5)c34)cc2)cc1 ; CC(C)(C)c1cc(-c2ccc(N3c4cc(-n5c6ccccc6c6ccccc65)cc5c4B(c4cc(-c6ccccc6)ccc4N5c4ccc(-c5ccccc5)cc4)c4c3c(-c3ccccc3)nn4-c3ccccc3)cc2)cc(C(C)(C)C)c1]

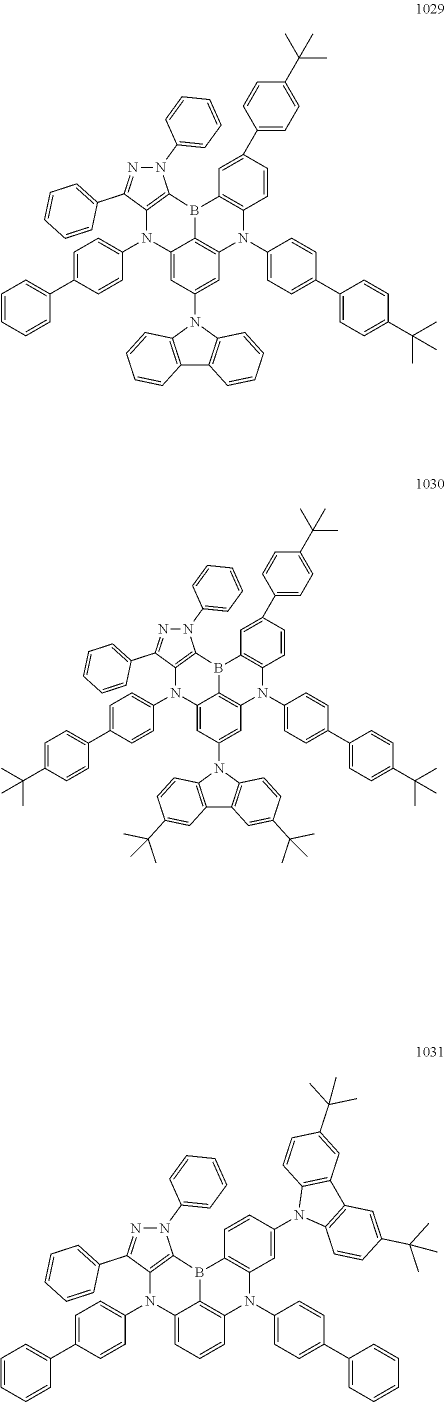

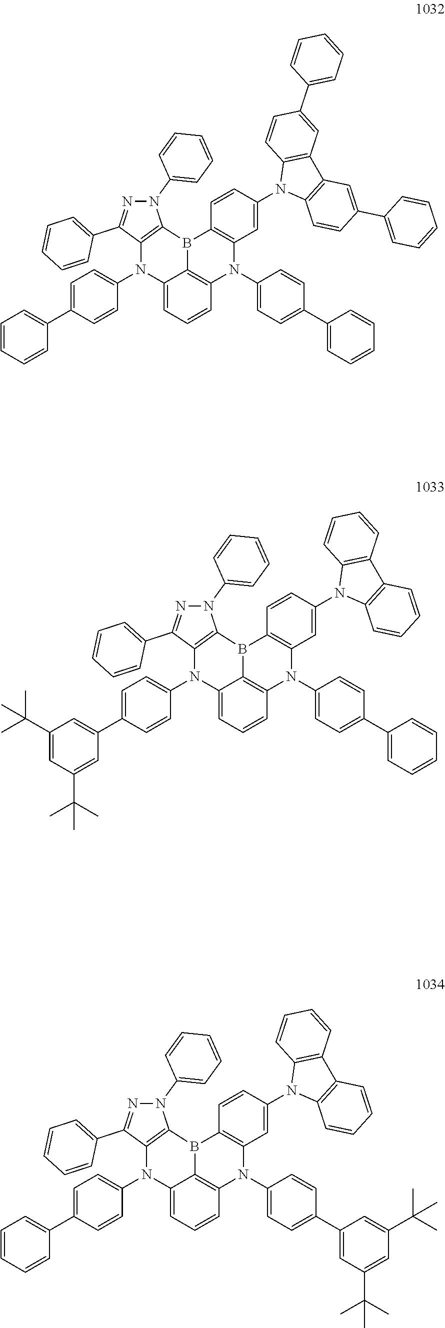 [SMILES: c1ccc(-c2ccc(N3c4cc(-n5c6ccc(-c7ccccc7)cc6c6cc(-c7ccccc7)ccc65)ccc4B4c5c3cccc5N(c3ccc(-c5ccccc5)cc3)c3c(-c5ccccc5)nn(-c5ccccc5)c34)cc2)cc1 ; CC(C)(C)c1cc(-c2ccc(N3c4cccc5c4B(c4ccc(-n6c7ccccc7c7ccccc76)cc4N5c4ccc(-c5ccccc5)cc4)c4c3c(-c3ccccc3)nn4-c3ccccc3)cc2)cc(C(C)(C)C)c1 ; CC(C)(C)c1cc(-c2ccc(N3c4cc(-n5c6ccccc6c6ccccc65)ccc4B4c5c3cccc5N(c3ccc(-c5ccccc5)cc3)c3c(-c5ccccc5)nn(-c5ccccc5)c34)cc2)cc(C(C)(C)C)c1]

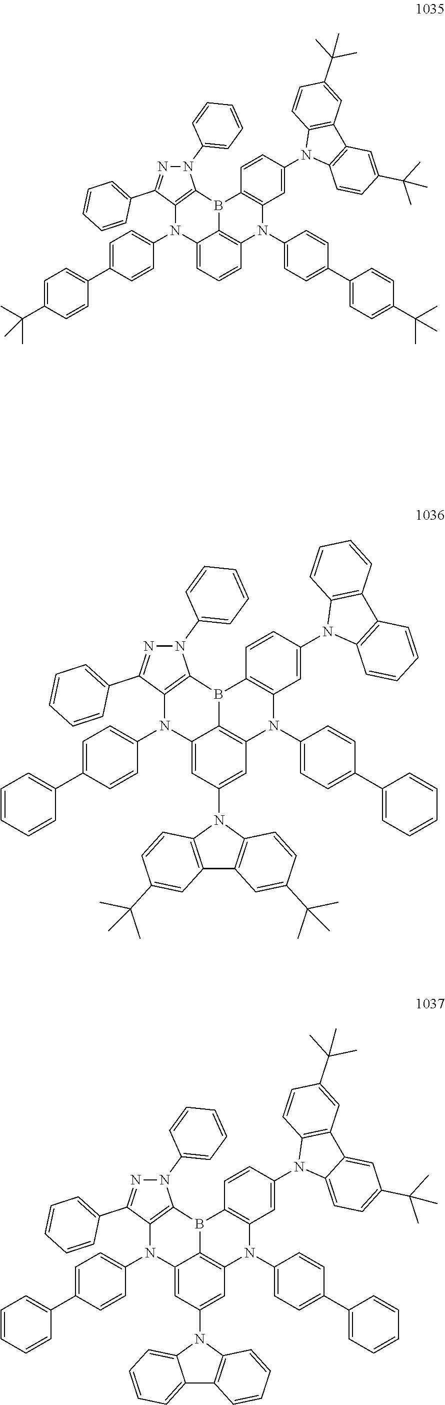 [SMILES: CC(C)(C)c1ccc(-c2ccc(N3c4cc(-n5c6ccc(C(C)(C)C)cc6c6cc(C(C)(C)C)ccc65)ccc4B4c5c3cccc5N(c3ccc(-c5ccc(C(C)(C)C)cc5)cc3)c3c(-c5ccccc5)nn(-c5ccccc5)c34)cc2)cc1 ; CC(C)(C)c1ccc2c(c1)c1cc(C(C)(C)C)ccc1n2-c1cc2c3c(c1)N(c1ccc(-c4ccccc4)cc1)c1c(-c4ccccc4)nn(-c4ccccc4)c1B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(c(-c2ccccc2)nn1-c1ccccc1)N3c1ccc(-c2ccccc2)cc1]

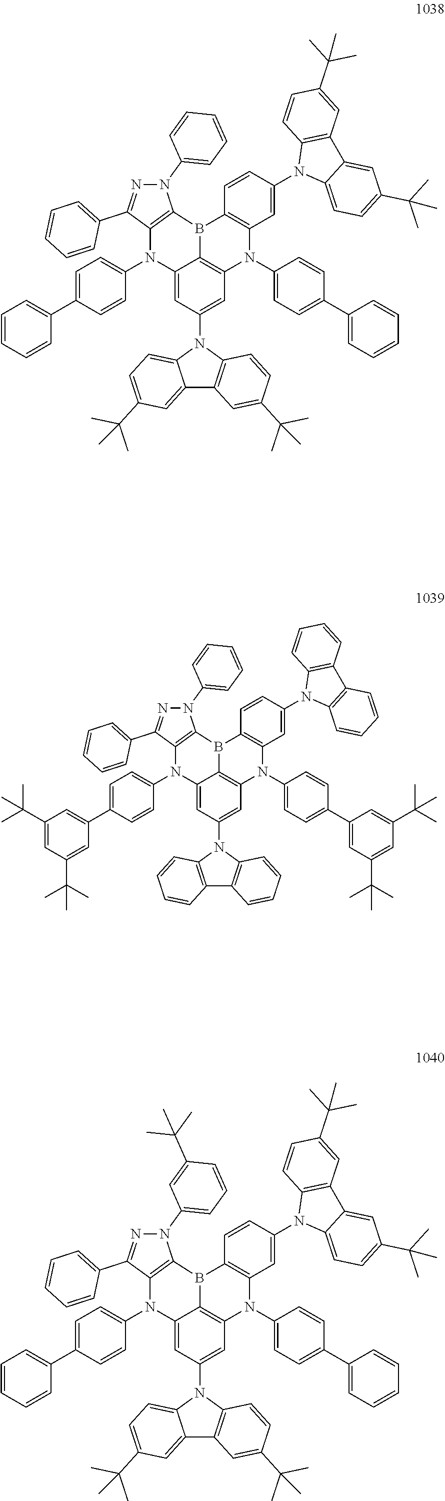

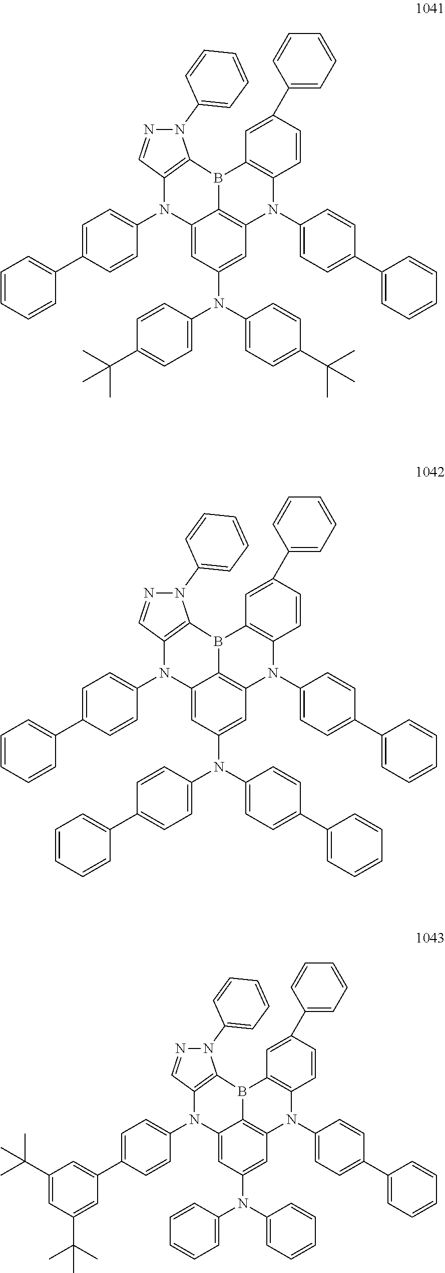 [SMILES: CC(C)(C)c1ccc(N(c2ccc(C(C)(C)C)cc2)c2cc3c4c(c2)N(c2ccc(-c5ccccc5)cc2)c2cnn(-c5ccccc5)c2B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3cnn(-c6ccccc6)c3B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c3cnn4-c3ccccc3)cc2)cc(C(C)(C)C)c1]

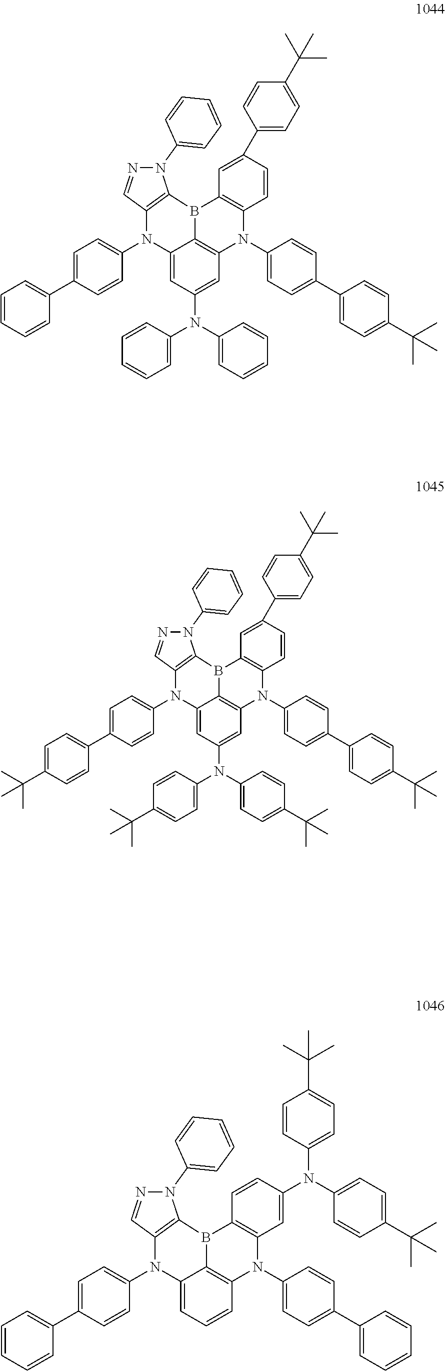

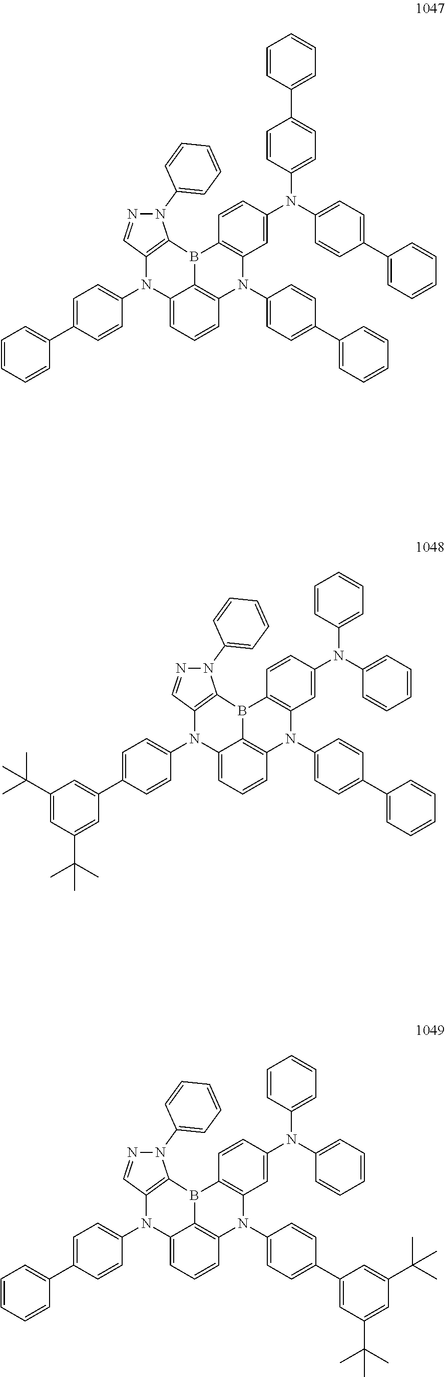 [SMILES: c1ccc(-c2ccc(N(c3ccc(-c4ccccc4)cc3)c3ccc4c(c3)N(c3ccc(-c5ccccc5)cc3)c3cccc5c3B4c3c(cnn3-c3ccccc3)N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4c3cnn4-c3ccccc3)cc2)cc(C(C)(C)C)c1 ; CC(C)(C)c1cc(-c2ccc(N3c4cc(N(c5ccccc5)c5ccccc5)ccc4B4c5c3cccc5N(c3ccc(-c5ccccc5)cc3)c3cnn(-c5ccccc5)c34)cc2)cc(C(C)(C)C)c1]

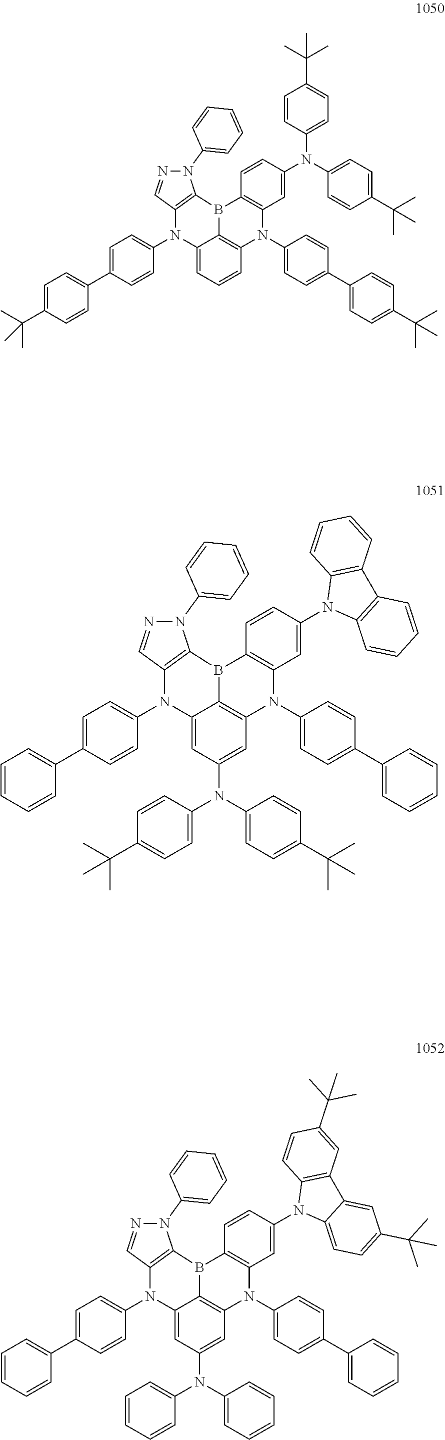 [SMILES: CC(C)(C)c1ccc(-c2ccc(N3c4cc(N(c5ccc(C(C)(C)C)cc5)c5ccc(C(C)(C)C)cc5)ccc4B4c5c3cccc5N(c3ccc(-c5ccc(C(C)(C)C)cc5)cc3)c3cnn(-c5ccccc5)c34)cc2)cc1 ; CC(C)(C)c1ccc(N(c2ccc(C(C)(C)C)cc2)c2cc3c4c(c2)N(c2ccc(-c5ccccc5)cc2)c2cnn(-c5ccccc5)c2B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(cnn1-c1ccccc1)N3c1ccc(-c2ccccc2)cc1]

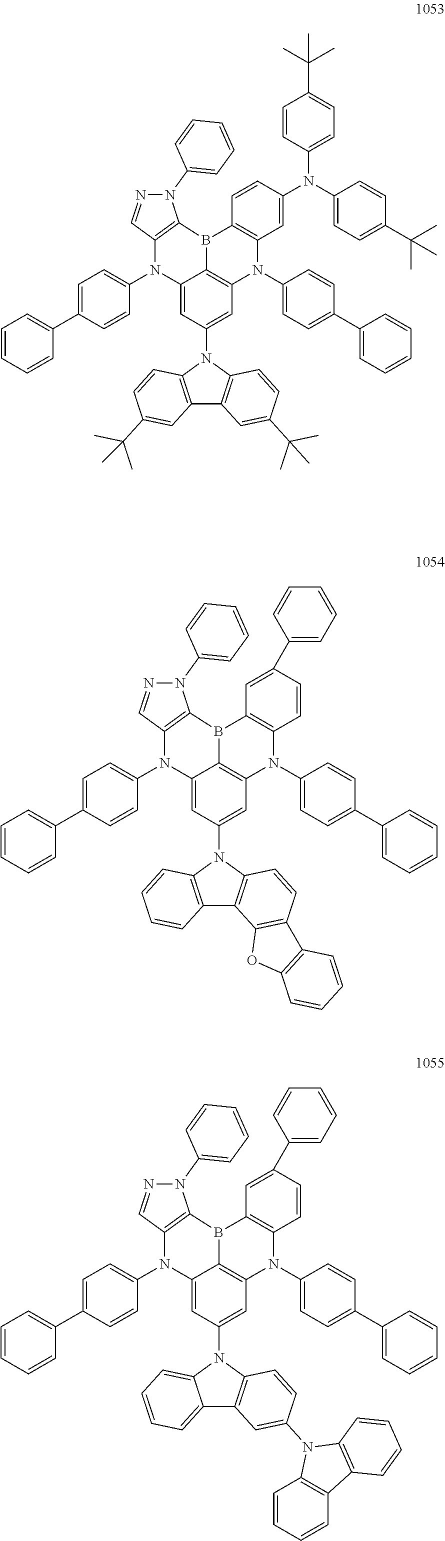 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2c(cnn2-c2ccccc2)N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3cnn(-c5ccccc5)c34)cc2)cc1 ; c1ccc(-c2ccc(N3c4ccc(-c5ccccc5)cc4B4c5c3cc(-n3c6ccccc6c6cc(-n7c8ccccc8c8ccccc87)ccc63)cc5N(c3ccc(-c5ccccc5)cc3)c3cnn(-c5ccccc5)c34)cc2)cc1]

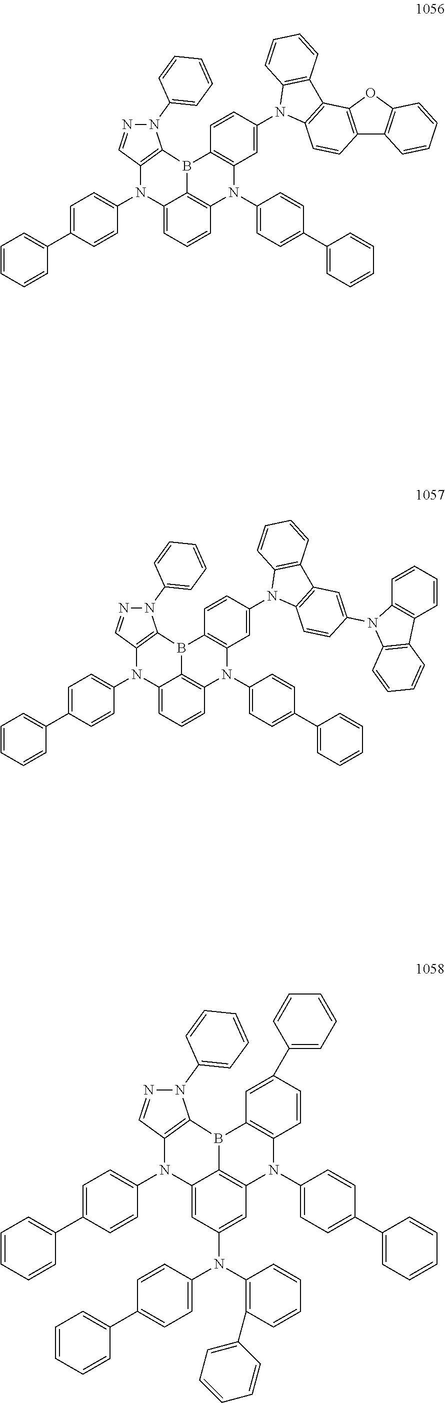 [SMILES: c1ccc(-c2ccc(N3c4cc(-n5c6ccccc6c6c7oc8ccccc8c7ccc65)ccc4B4c5c3cccc5N(c3ccc(-c5ccccc5)cc3)c3cnn(-c5ccccc5)c34)cc2)cc1 ; c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3cnn(-c5ccccc5)c34)cc2)cc1 ; c1ccc(-c2ccc(N(c3cc4c5c(c3)N(c3ccc(-c6ccccc6)cc3)c3cnn(-c6ccccc6)c3B5c3cc(-c5ccccc5)ccc3N4c3ccc(-c4ccccc4)cc3)c3ccccc3-c3ccccc3)cc2)cc1]

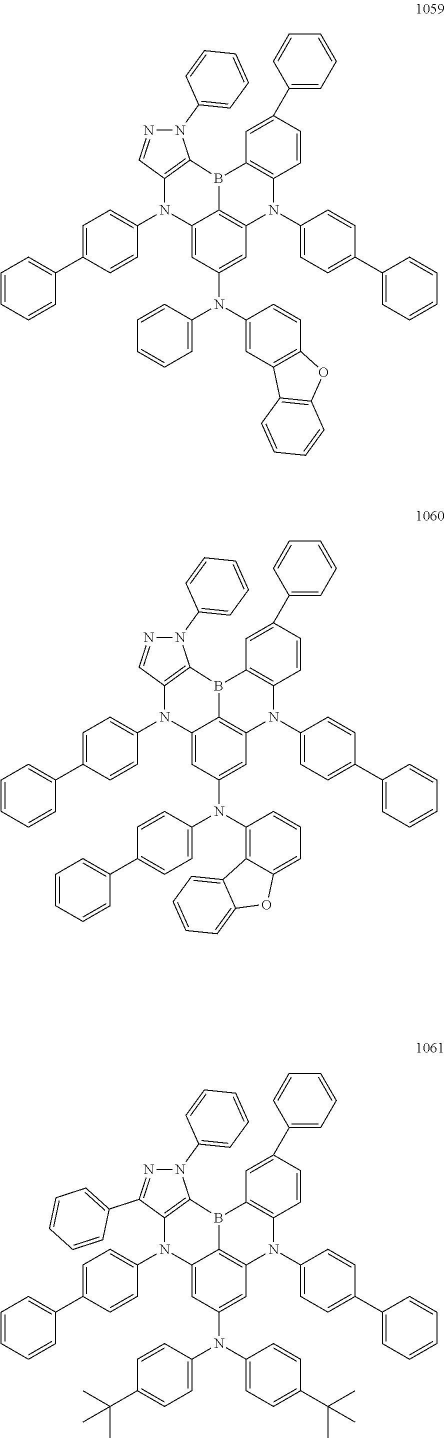 [SMILES: c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3cnn(-c5ccccc5)c34)cc2)cc1 ; c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3cnn(-c5ccccc5)c34)cc2)cc1 ; CC(C)(C)c1ccc(N(c2ccc(C(C)(C)C)cc2)c2cc3c4c(c2)N(c2ccc(-c5ccccc5)cc2)c2c(-c5ccccc5)nn(-c5ccccc5)c2B4c2cc(-c4ccccc4)ccc2N3c2ccc(-c3ccccc3)cc2)cc1]

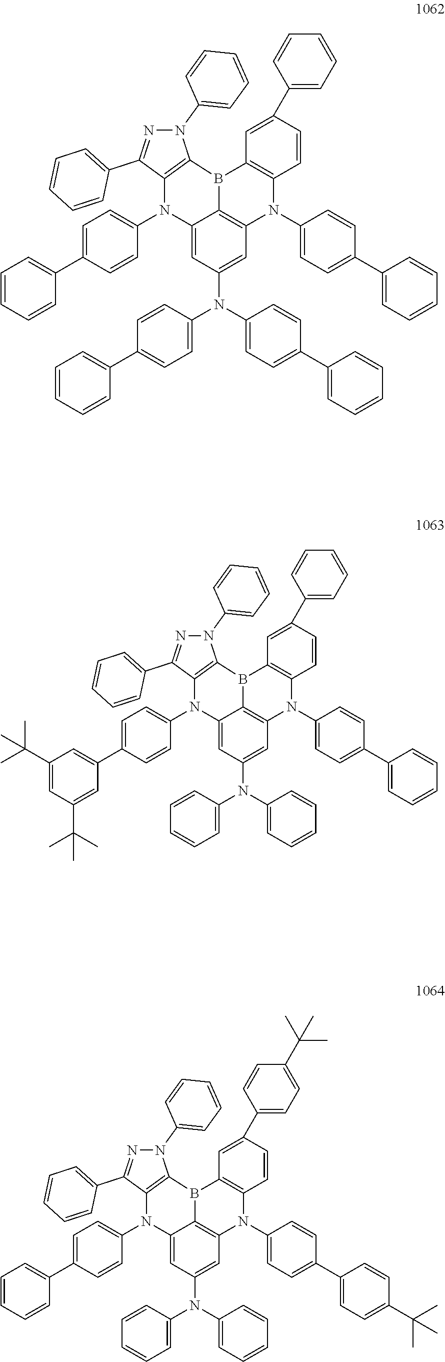 [SMILES: c1ccc(-c2ccc(N(c3ccc(-c4ccccc4)cc3)c3cc4c5c(c3)N(c3ccc(-c6ccccc6)cc3)c3c(-c6ccccc6)nn(-c6ccccc6)c3B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c3c(-c3ccccc3)nn4-c3ccccc3)cc2)cc(C(C)(C)C)c1 ; CC(C)(C)c1ccc(-c2ccc(N3c4ccc(-c5ccc(C(C)(C)C)cc5)cc4B4c5c3cc(N(c3ccccc3)c3ccccc3)cc5N(c3ccc(-c5ccccc5)cc3)c3c(-c5ccccc5)nn(-c5ccccc5)c34)cc2)cc1]

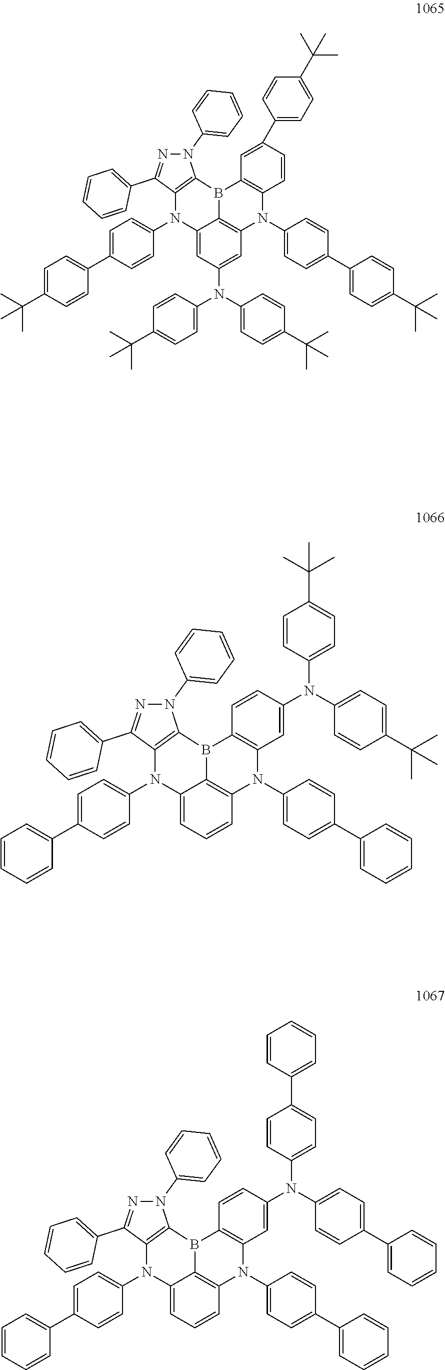 [SMILES: CC(C)(C)c1ccc(-c2ccc(N3c4ccc(-c5ccc(C(C)(C)C)cc5)cc4B4c5c3cc(N(c3ccc(C(C)(C)C)cc3)c3ccc(C(C)(C)C)cc3)cc5N(c3ccc(-c5ccc(C(C)(C)C)cc5)cc3)c3c(-c5ccccc5)nn(-c5ccccc5)c34)cc2)cc1 ; CC(C)(C)c1ccc(N(c2ccc(C(C)(C)C)cc2)c2ccc3c(c2)N(c2ccc(-c4ccccc4)cc2)c2cccc4c2B3c2c(c(-c3ccccc3)nn2-c2ccccc2)N4c2ccc(-c3ccccc3)cc2)cc1 ; c1ccc(-c2ccc(N(c3ccc(-c4ccccc4)cc3)c3ccc4c(c3)N(c3ccc(-c5ccccc5)cc3)c3cccc5c3B4c3c(c(-c4ccccc4)nn3-c3ccccc3)N5c3ccc(-c4ccccc4)cc3)cc2)cc1]

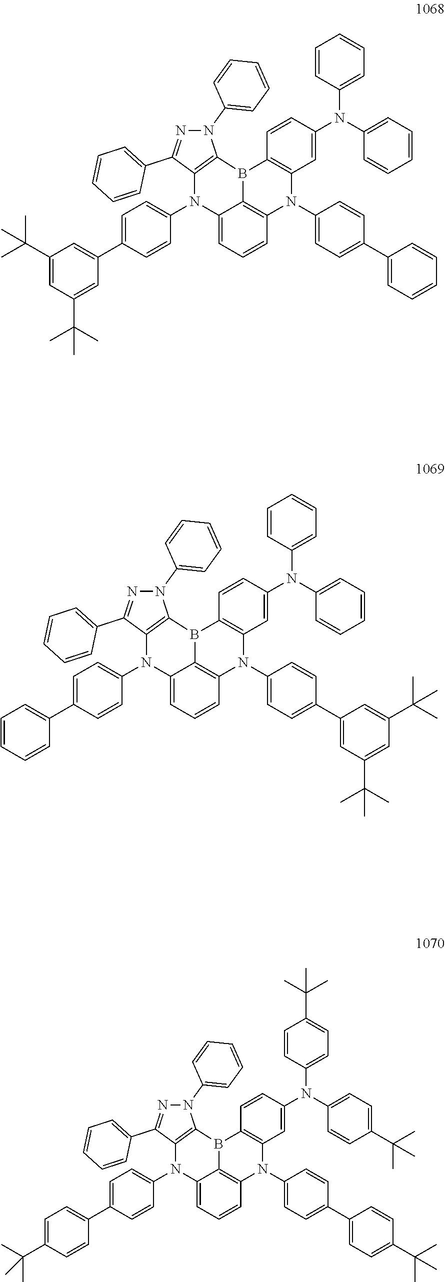

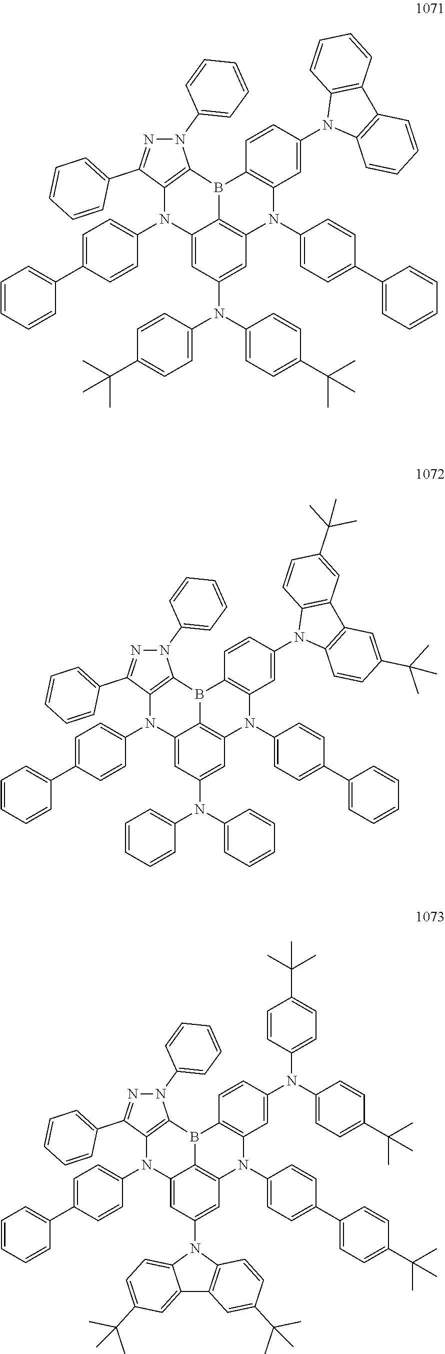

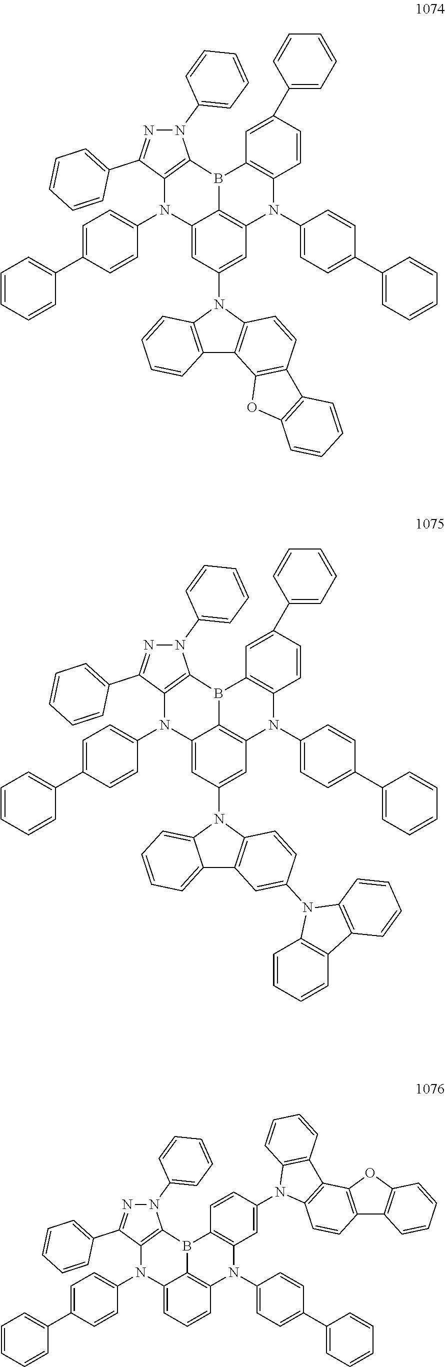 [SMILES: c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3c(-c5ccccc5)nn(-c5ccccc5)c34)cc2)cc1 ; c1ccc(-c2ccc(N3c4ccc(-c5ccccc5)cc4B4c5c3cc(-n3c6ccccc6c6cc(-n7c8ccccc8c8ccccc87)ccc63)cc5N(c3ccc(-c5ccccc5)cc3)c3c(-c5ccccc5)nn(-c5ccccc5)c34)cc2)cc1 ; c1ccc(-c2ccc(N3c4cc(-n5c6ccccc6c6c7oc8ccccc8c7ccc65)ccc4B4c5c3cccc5N(c3ccc(-c5ccccc5)cc3)c3c(-c5ccccc5)nn(-c5ccccc5)c34)cc2)cc1]

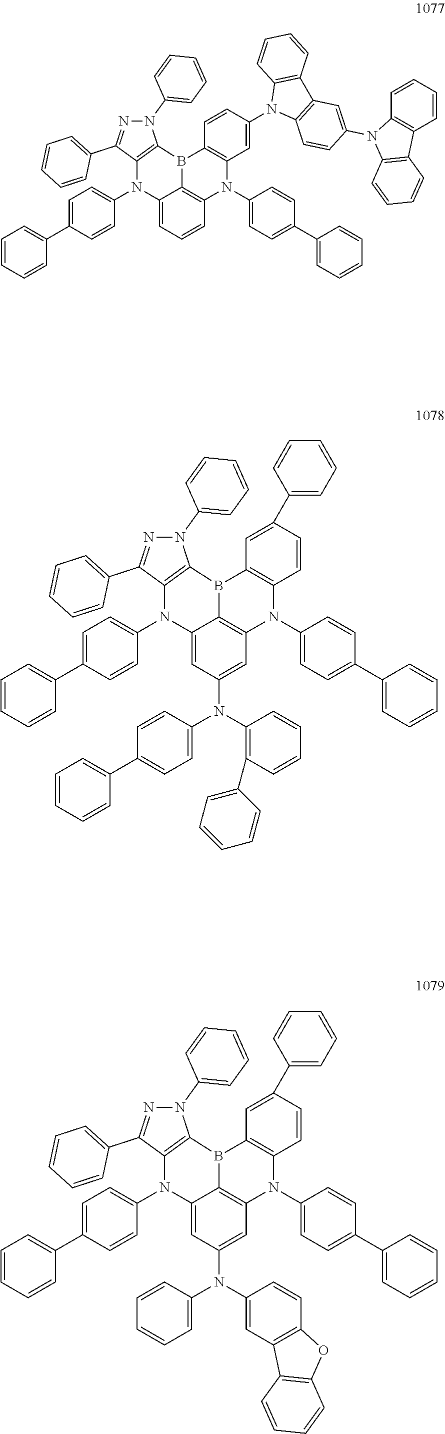 [SMILES: c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3c(-c5ccccc5)nn(-c5ccccc5)c34)cc2)cc1 ; c1ccc(-c2ccc(N(c3cc4c5c(c3)N(c3ccc(-c6ccccc6)cc3)c3c(-c6ccccc6)nn(-c6ccccc6)c3B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3c(-c5ccccc5)nn(-c5ccccc5)c34)cc2)cc1]

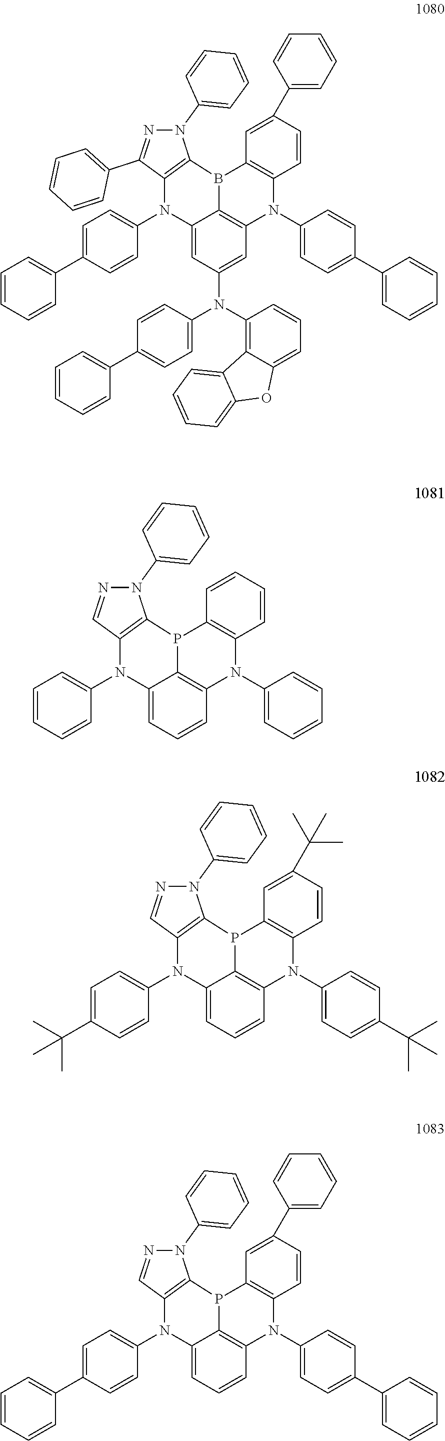 [SMILES: c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3c(-c5ccccc5)nn(-c5ccccc5)c34)cc2)cc1 ; c1ccc(N2c3ccccc3P3c4c2cccc4N(c2ccccc2)c2cnn(-c4ccccc4)c23)cc1 ; CC(C)(C)c1ccc(N2c3ccc(C(C)(C)C)cc3P3c4c2cccc4N(c2ccc(C(C)(C)C)cc2)c2cnn(-c4ccccc4)c23)cc1 ; c1ccc(-c2ccc(N3c4ccc(-c5ccccc5)cc4P4c5c3cccc5N(c3ccc(-c5ccccc5)cc3)c3cnn(-c5ccccc5)c34)cc2)cc1]

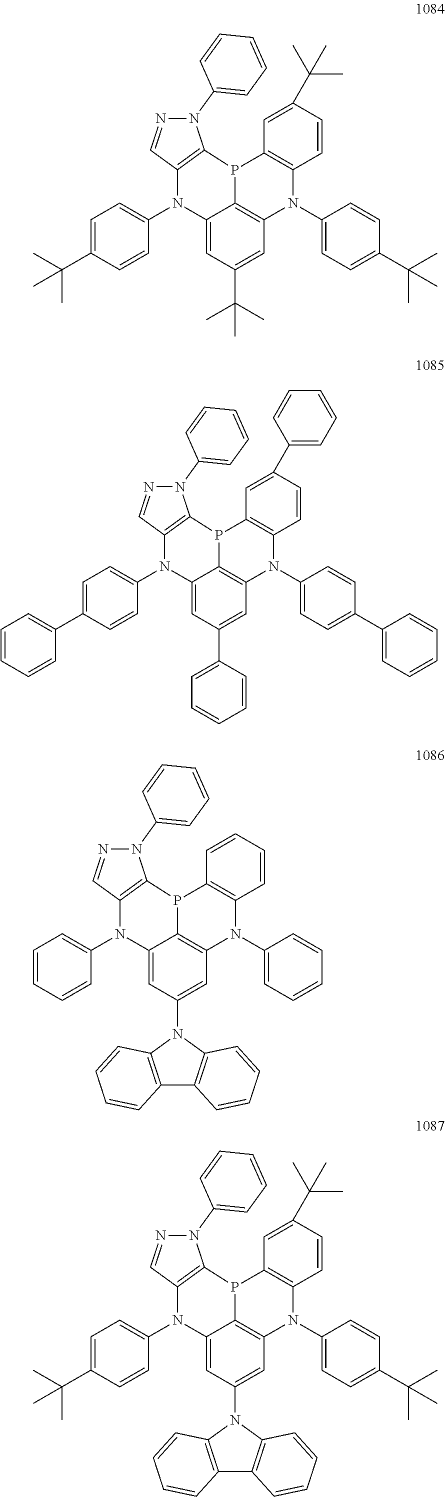

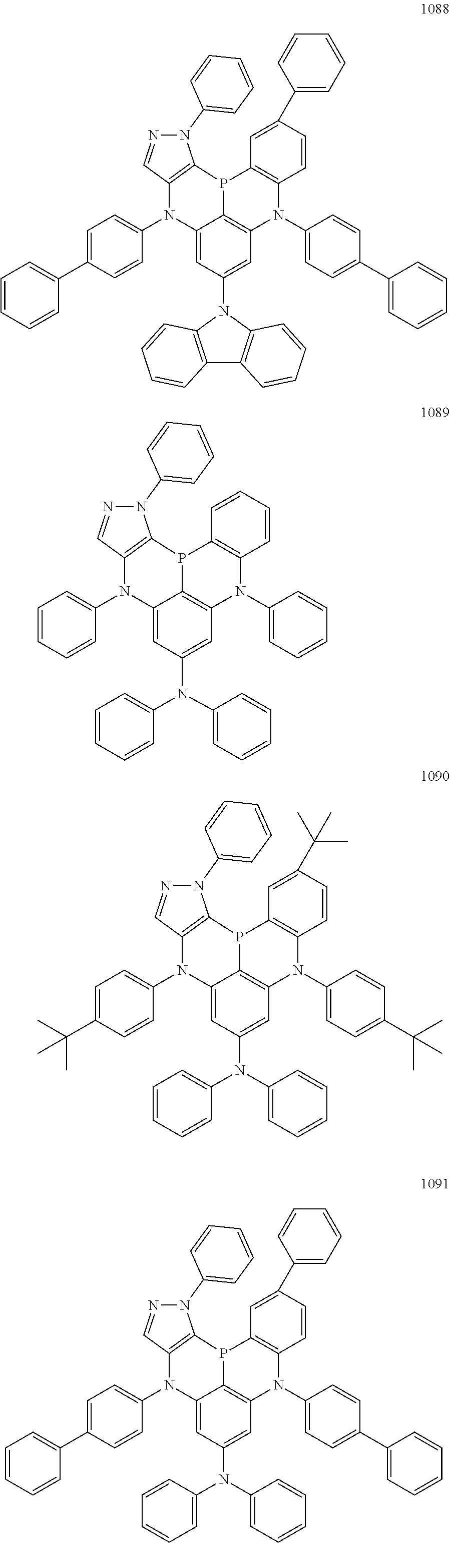

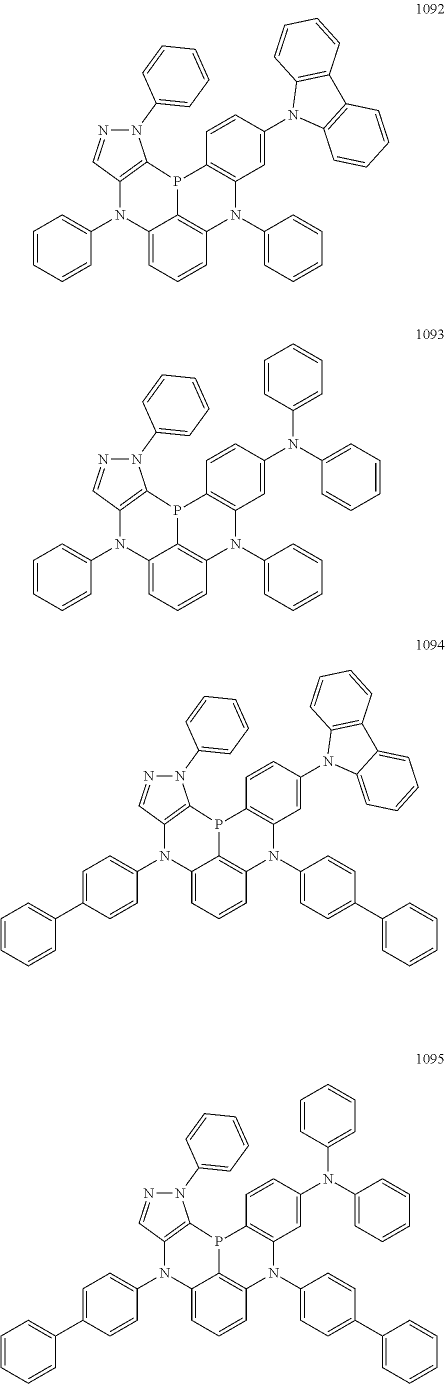 [SMILES: c1ccc(N2c3cc(-n4c5ccccc5c5ccccc54)ccc3P3c4c2cccc4N(c2ccccc2)c2cnn(-c4ccccc4)c23)cc1 ; c1ccc(N(c2ccccc2)c2ccc3c(c2)N(c2ccccc2)c2cccc4c2P3c2c(cnn2-c2ccccc2)N4c2ccccc2)cc1 ; c1ccc(-c2ccc(N3c4cc(-n5c6ccccc6c6ccccc65)ccc4P4c5c3cccc5N(c3ccc(-c5ccccc5)cc3)c3cnn(-c5ccccc5)c34)cc2)cc1 ; c1ccc(-c2ccc(N3c4cc(N(c5ccccc5)c5ccccc5)ccc4P4c5c3cccc5N(c3ccc(-c5ccccc5)cc3)c3cnn(-c5ccccc5)c34)cc2)cc1]

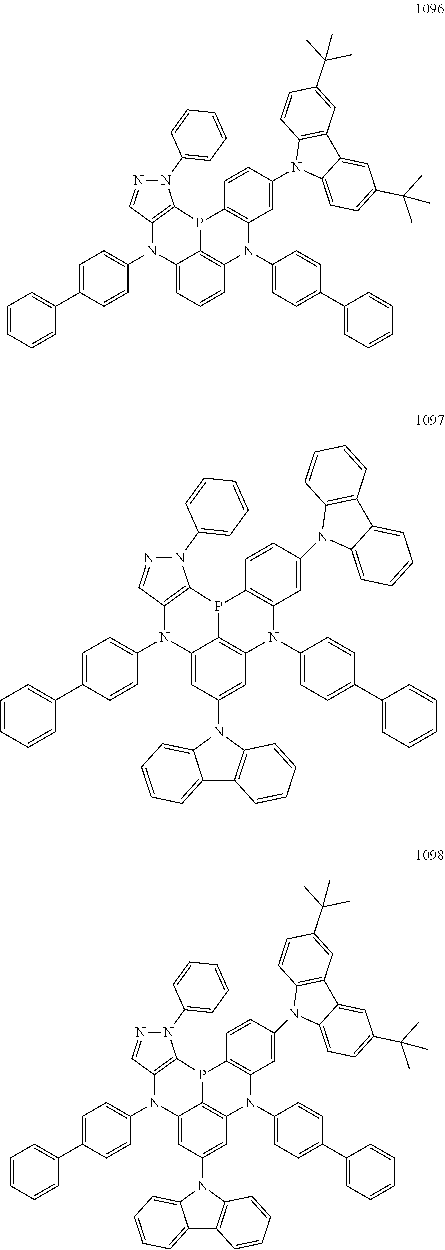 [SMILES: CC(C)(C)c1ccc2c(c1)c1cc(C(C)(C)C)ccc1n2-c1ccc2c(c1)N(c1ccc(-c3ccccc3)cc1)c1cccc3c1P2c1c(cnn1-c1ccccc1)N3c1ccc(-c2ccccc2)cc1 ; c1ccc(-c2ccc(N3c4cc(-n5c6ccccc6c6ccccc65)ccc4P4c5c3cc(-n3c6ccccc6c6ccccc63)cc5N(c3ccc(-c5ccccc5)cc3)c3cnn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1c(cnn1-c1ccccc1)N3c1ccc(-c2ccccc2)cc1]

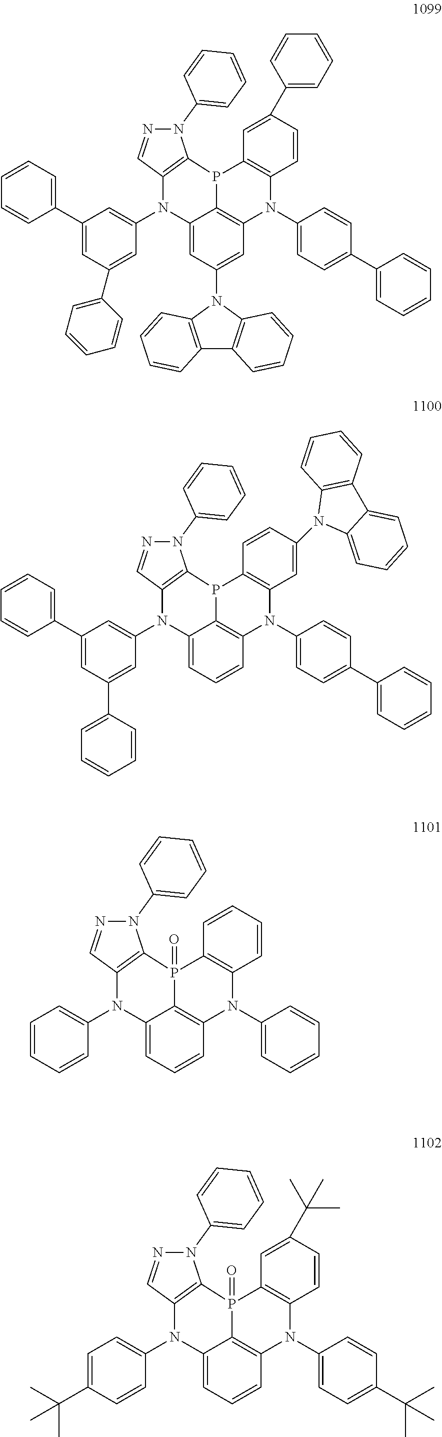 [SMILES: c1ccc(-c2ccc(N3c4ccc(-c5ccccc5)cc4P4c5c3cc(-n3c6ccccc6c6ccccc63)cc5N(c3cc(-c5ccccc5)cc(-c5ccccc5)c3)c3cnn(-c5ccccc5)c34)cc2)cc1 ; c1ccc(-c2ccc(N3c4cc(-n5c6ccccc6c6ccccc65)ccc4P4c5c3cccc5N(c3cc(-c5ccccc5)cc(-c5ccccc5)c3)c3cnn(-c5ccccc5)c34)cc2)cc1 ; O=P12c3ccccc3N(c3ccccc3)c3cccc(c31)N(c1ccccc1)c1cnn(-c3ccccc3)c12 ; CC(C)(C)c1ccc(N2c3ccc(C(C)(C)C)cc3P3(=O)c4c2cccc4N(c2ccc(C(C)(C)C)cc2)c2cnn(-c4ccccc4)c23)cc1]

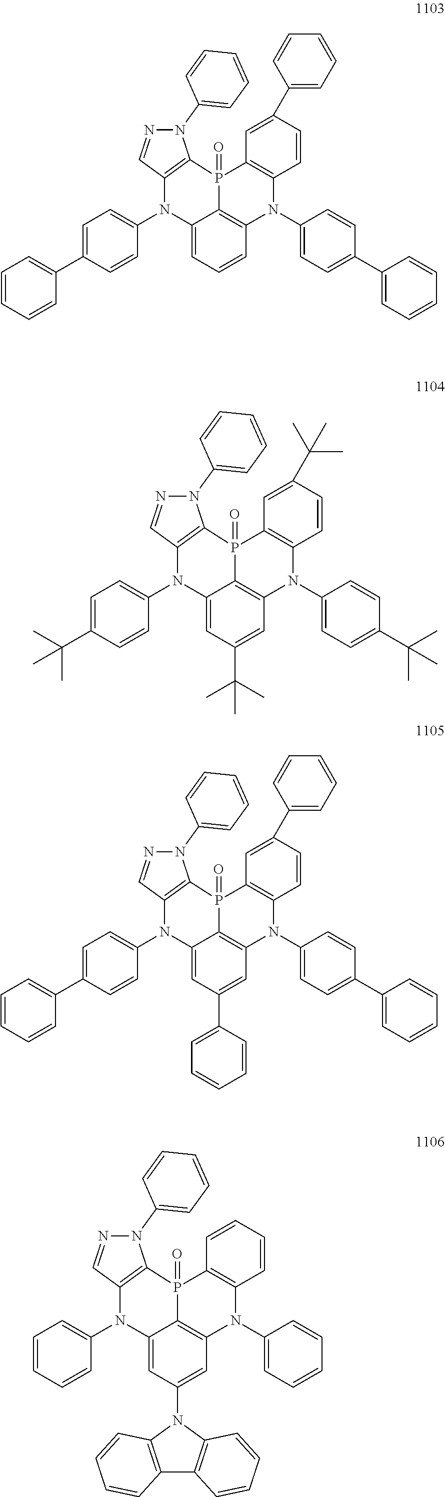 [SMILES: O=P12c3cc(-c4ccccc4)ccc3N(c3ccc(-c4ccccc4)cc3)c3cccc(c31)N(c1ccc(-c3ccccc3)cc1)c1cnn(-c3ccccc3)c12 ; CC(C)(C)c1ccc(N2c3ccc(C(C)(C)C)cc3P3(=O)c4c2cc(C(C)(C)C)cc4N(c2ccc(C(C)(C)C)cc2)c2cnn(-c4ccccc4)c23)cc1 ; O=P12c3cc(-c4ccccc4)ccc3N(c3ccc(-c4ccccc4)cc3)c3cc(-c4ccccc4)cc(c31)N(c1ccc(-c3ccccc3)cc1)c1cnn(-c3ccccc3)c12 ; O=P12c3ccccc3N(c3ccccc3)c3cc(-n4c5ccccc5c5ccccc54)cc(c31)N(c1ccccc1)c1cnn(-c3ccccc3)c12]

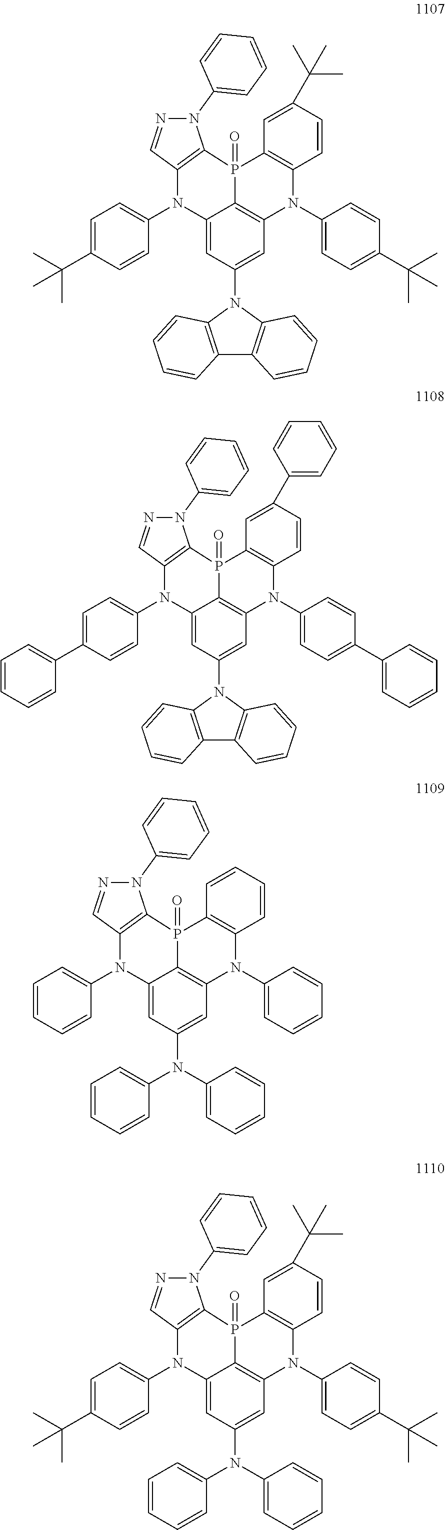

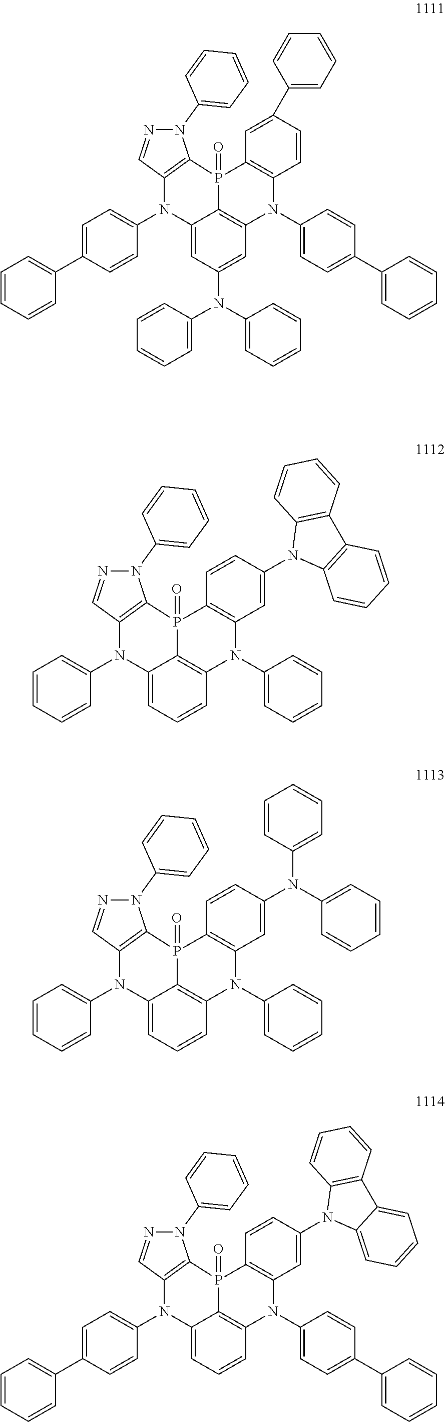 [SMILES: O=P12c3cc(-c4ccccc4)ccc3N(c3ccc(-c4ccccc4)cc3)c3cc(N(c4ccccc4)c4ccccc4)cc(c31)N(c1ccc(-c3ccccc3)cc1)c1cnn(-c3ccccc3)c12 ; O=P12c3ccc(-n4c5ccccc5c5ccccc54)cc3N(c3ccccc3)c3cccc(c31)N(c1ccccc1)c1cnn(-c3ccccc3)c12 ; O=P12c3ccc(N(c4ccccc4)c4ccccc4)cc3N(c3ccccc3)c3cccc(c31)N(c1ccccc1)c1cnn(-c3ccccc3)c12 ; O=P12c3ccc(-n4c5ccccc5c5ccccc54)cc3N(c3ccc(-c4ccccc4)cc3)c3cccc(c31)N(c1ccc(-c3ccccc3)cc1)c1cnn(-c3ccccc3)c12]

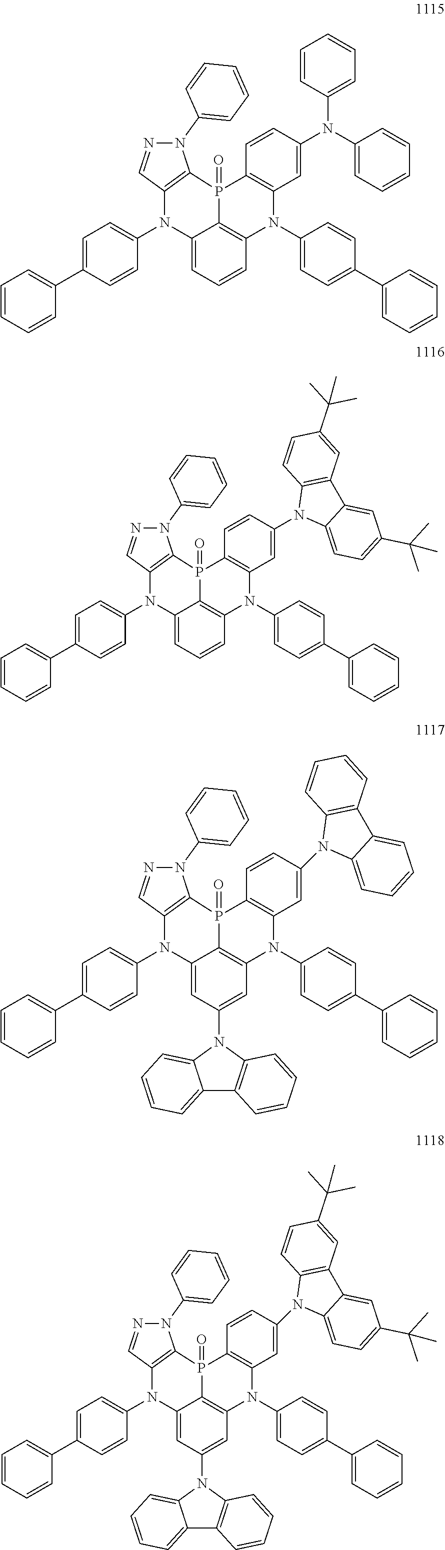

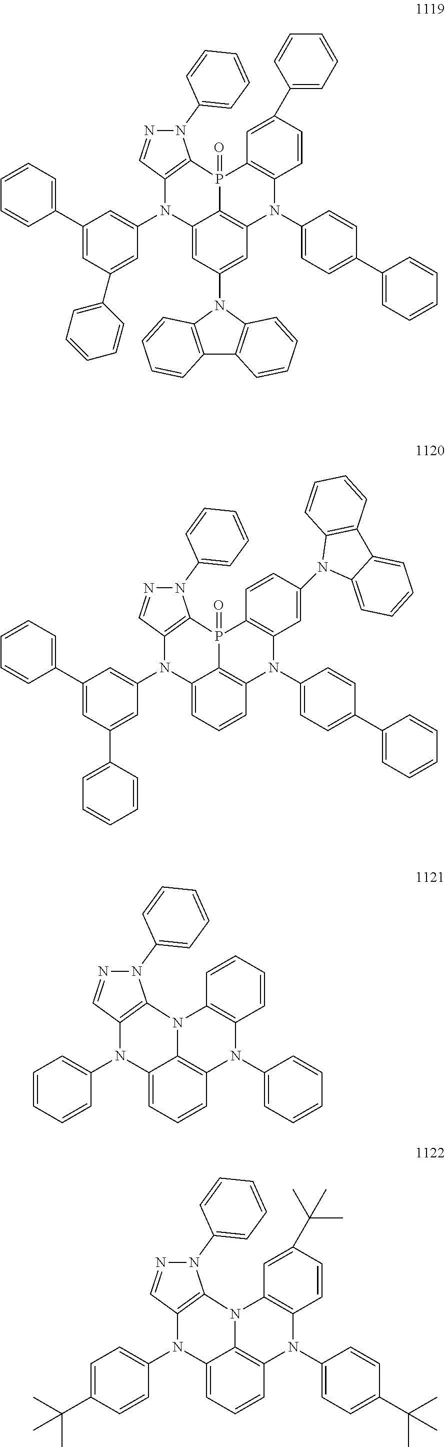 [SMILES: O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1cnn(-c3ccccc3)c12 ; O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1cnn(-c3ccccc3)c12 ; c1ccc(N2c3ccccc3N3c4c2cccc4N(c2ccccc2)c2cnn(-c4ccccc4)c23)cc1 ; CC(C)(C)c1ccc(N2c3ccc(C(C)(C)C)cc3N3c4c2cccc4N(c2ccc(C(C)(C)C)cc2)c2cnn(-c4ccccc4)c23)cc1]

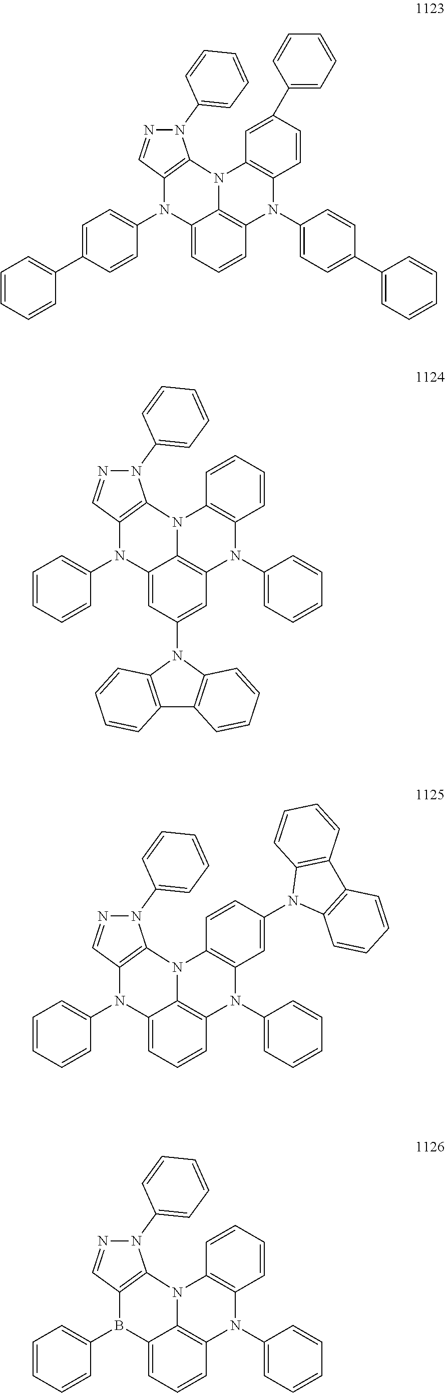 [SMILES: c1ccc(-c2ccc(N3c4ccc(-c5ccccc5)cc4N4c5c3cccc5N(c3ccc(-c5ccccc5)cc3)c3cnn(-c5ccccc5)c34)cc2)cc1 ; c1ccc(N2c3ccccc3N3c4c2cc(-n2c5ccccc5c5ccccc52)cc4N(c2ccccc2)c2cnn(-c4ccccc4)c23)cc1 ; c1ccc(N2c3cc(-n4c5ccccc5c5ccccc54)ccc3N3c4c2cccc4N(c2ccccc2)c2cnn(-c4ccccc4)c23)cc1 ; c1ccc(B2c3cccc4c3N(c3ccccc3N4c3ccccc3)c3c2cnn3-c2ccccc2)cc1]

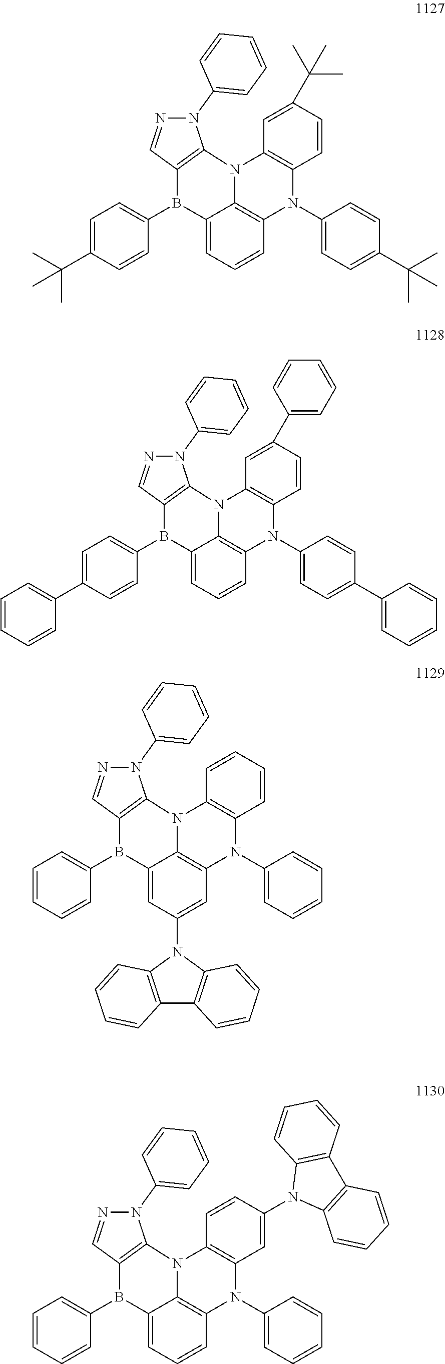 [SMILES: CC(C)(C)c1ccc(B2c3cccc4c3N(c3cc(C(C)(C)C)ccc3N4c3ccc(C(C)(C)C)cc3)c3c2cnn3-c2ccccc2)cc1 ; c1ccc(-c2ccc(B3c4cccc5c4N(c4cc(-c6ccccc6)ccc4N5c4ccc(-c5ccccc5)cc4)c4c3cnn4-c3ccccc3)cc2)cc1 ; c1ccc(B2c3cc(-n4c5ccccc5c5ccccc54)cc4c3N(c3ccccc3N4c3ccccc3)c3c2cnn3-c2ccccc2)cc1 ; c1ccc(B2c3cccc4c3N(c3ccc(-n5c6ccccc6c6ccccc65)cc3N4c3ccccc3)c3c2cnn3-c2ccccc2)cc1]

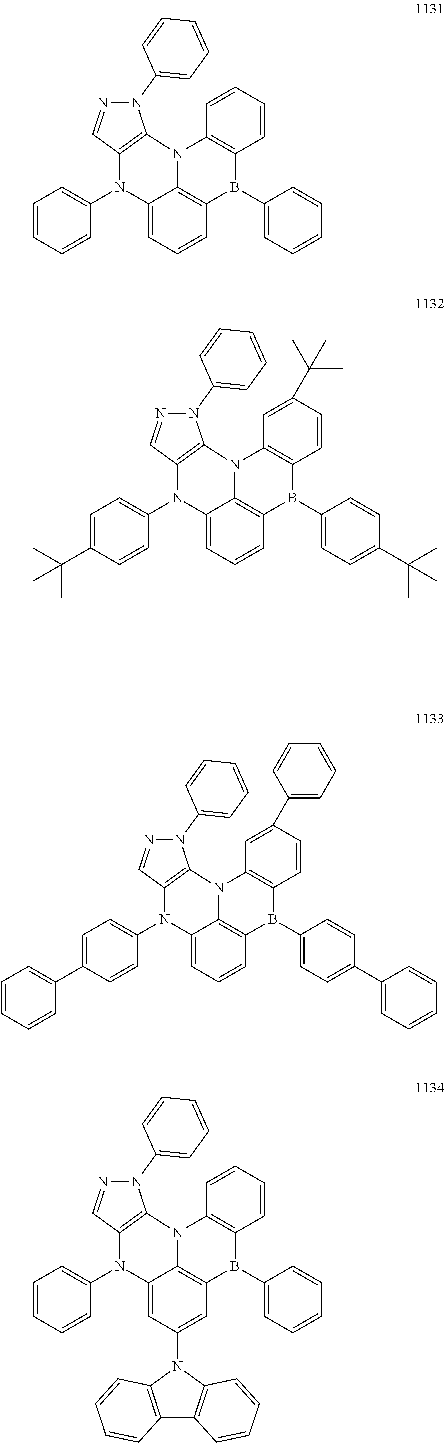 [SMILES: c1ccc(B2c3ccccc3N3c4c2cccc4N(c2ccccc2)c2cnn(-c4ccccc4)c23)cc1 ; CC(C)(C)c1ccc(B2c3ccc(C(C)(C)C)cc3N3c4c2cccc4N(c2ccc(C(C)(C)C)cc2)c2cnn(-c4ccccc4)c23)cc1 ; c1ccc(-c2ccc(B3c4ccc(-c5ccccc5)cc4N4c5c3cccc5N(c3ccc(-c5ccccc5)cc3)c3cnn(-c5ccccc5)c34)cc2)cc1 ; c1ccc(B2c3ccccc3N3c4c2cc(-n2c5ccccc5c5ccccc52)cc4N(c2ccccc2)c2cnn(-c4ccccc4)c23)cc1]

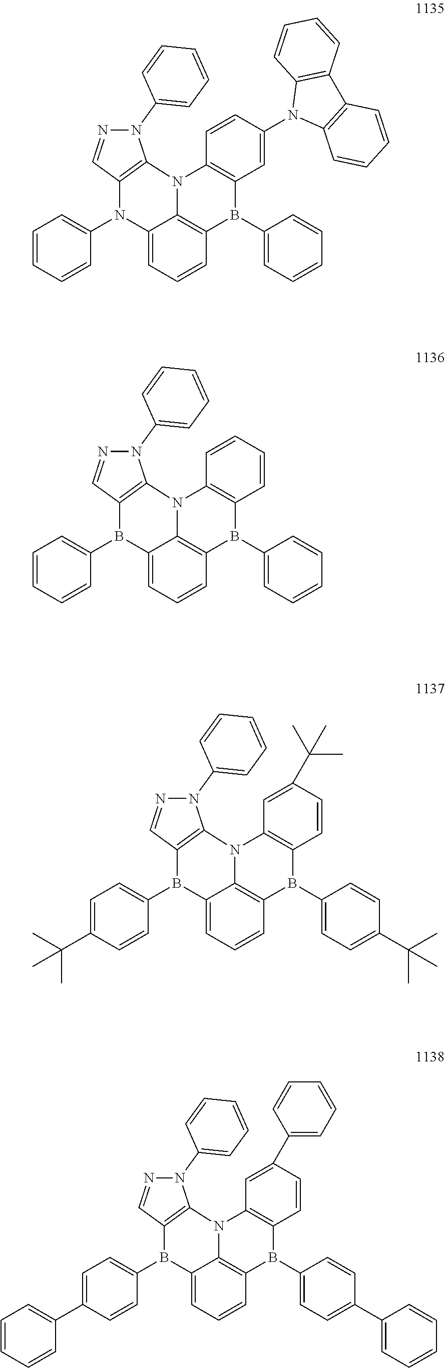 [SMILES: c1ccc(B2c3cc(-n4c5ccccc5c5ccccc54)ccc3N3c4c2cccc4N(c2ccccc2)c2cnn(-c4ccccc4)c23)cc1 ; c1ccc(B2c3ccccc3N3c4c2cccc4B(c2ccccc2)c2cnn(-c4ccccc4)c23)cc1 ; CC(C)(C)c1ccc(B2c3ccc(C(C)(C)C)cc3N3c4c2cccc4B(c2ccc(C(C)(C)C)cc2)c2cnn(-c4ccccc4)c23)cc1 ; c1ccc(-c2ccc(B3c4ccc(-c5ccccc5)cc4N4c5c3cccc5B(c3ccc(-c5ccccc5)cc3)c3cnn(-c5ccccc5)c34)cc2)cc1]

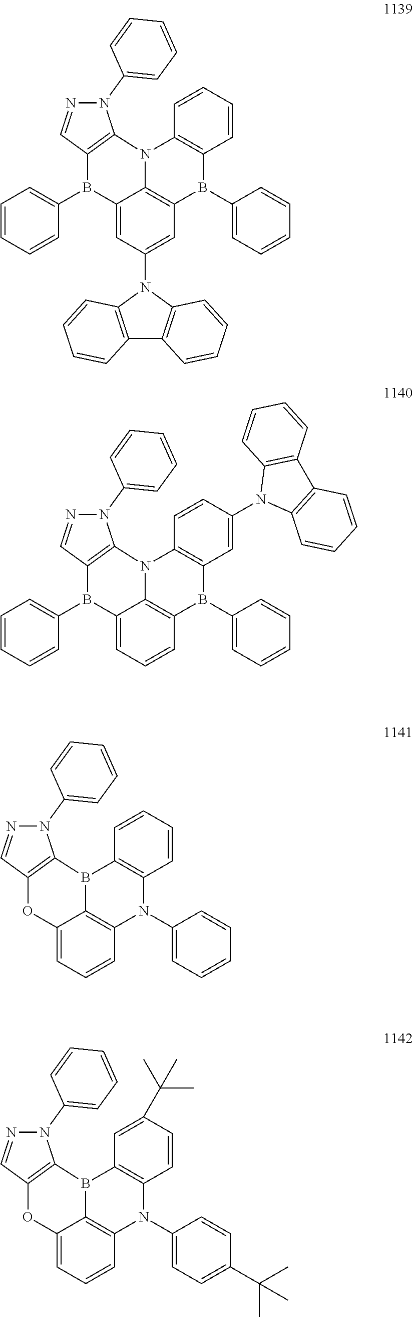 [SMILES: c1ccc(B2c3ccccc3N3c4c2cc(-n2c5ccccc5c5ccccc52)cc4B(c2ccccc2)c2cnn(-c4ccccc4)c23)cc1 ; c1ccc(B2c3cc(-n4c5ccccc5c5ccccc54)ccc3N3c4c2cccc4B(c2ccccc2)c2cnn(-c4ccccc4)c23)cc1 ; c1ccc(N2c3ccccc3B3c4c(cccc42)Oc2cnn(-c4ccccc4)c23)cc1 ; CC(C)(C)c1ccc(N2c3ccc(C(C)(C)C)cc3B3c4c(cccc42)Oc2cnn(-c4ccccc4)c23)cc1]

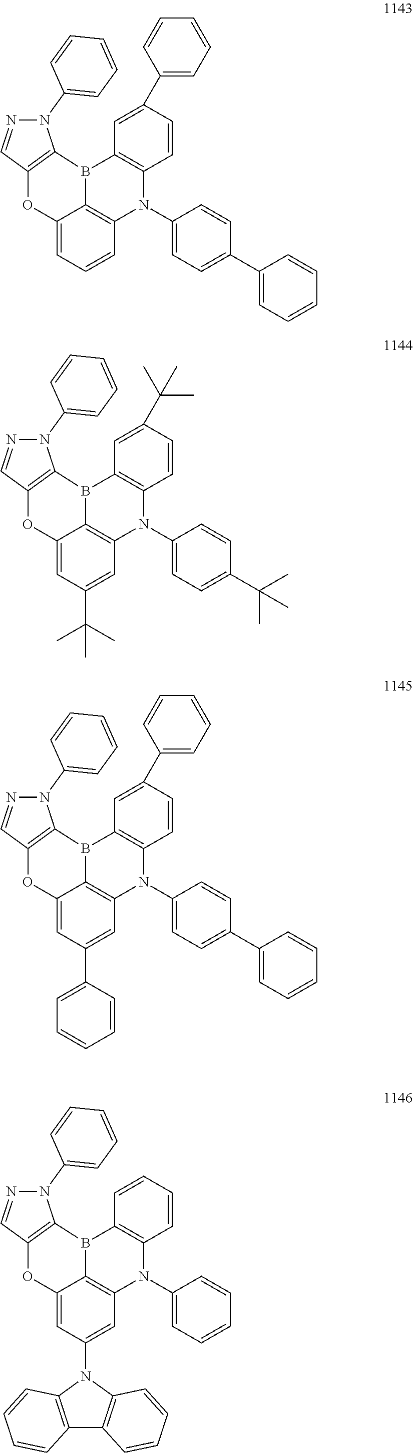 [SMILES: c1ccc(-c2ccc(N3c4ccc(-c5ccccc5)cc4B4c5c(cccc53)Oc3cnn(-c5ccccc5)c34)cc2)cc1 ; CC(C)(C)c1ccc(N2c3ccc(C(C)(C)C)cc3B3c4c(cc(C(C)(C)C)cc42)Oc2cnn(-c4ccccc4)c23)cc1 ; c1ccc(-c2ccc(N3c4ccc(-c5ccccc5)cc4B4c5c(cc(-c6ccccc6)cc53)Oc3cnn(-c5ccccc5)c34)cc2)cc1 ; c1ccc(N2c3ccccc3B3c4c(cc(-n5c6ccccc6c6ccccc65)cc42)Oc2cnn(-c4ccccc4)c23)cc1]

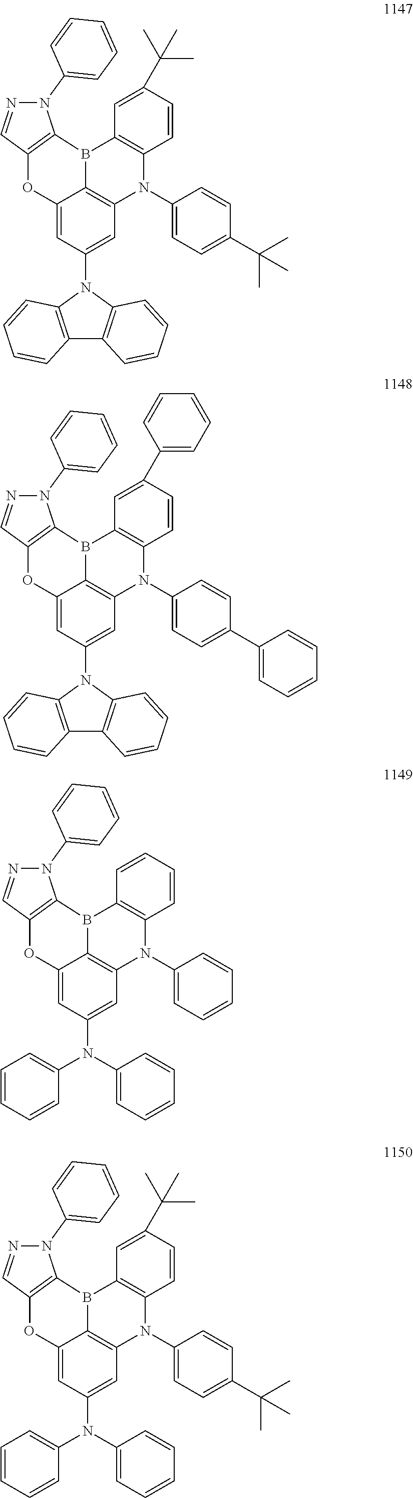

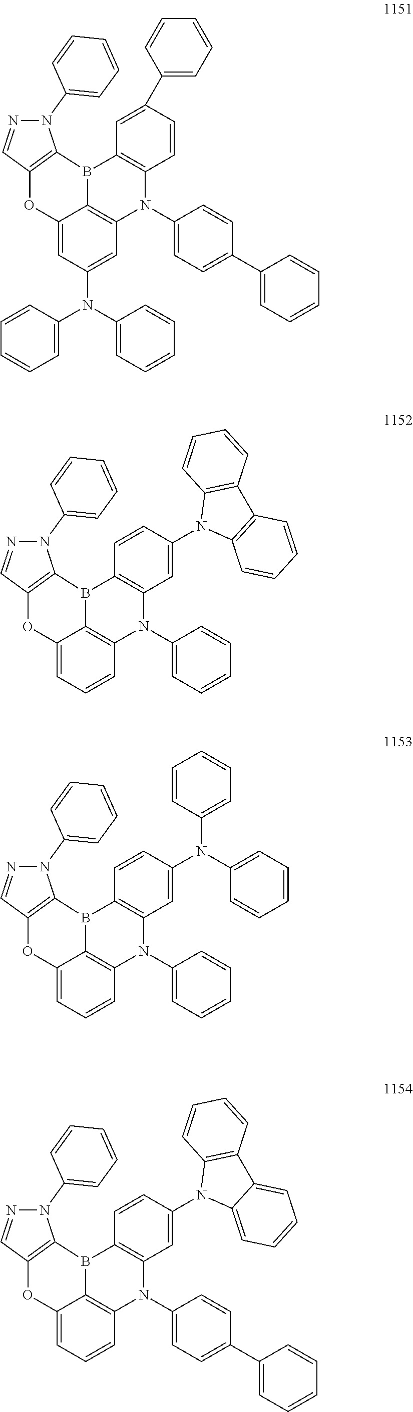 [SMILES: c1ccc(-c2ccc(N3c4ccc(-c5ccccc5)cc4B4c5c(cc(N(c6ccccc6)c6ccccc6)cc53)Oc3cnn(-c5ccccc5)c34)cc2)cc1 ; c1ccc(N2c3cc(-n4c5ccccc5c5ccccc54)ccc3B3c4c(cccc42)Oc2cnn(-c4ccccc4)c23)cc1 ; c1ccc(N(c2ccccc2)c2ccc3c(c2)N(c2ccccc2)c2cccc4c2B3c2c(cnn2-c2ccccc2)O4)cc1 ; c1ccc(-c2ccc(N3c4cc(-n5c6ccccc6c6ccccc65)ccc4B4c5c(cccc53)Oc3cnn(-c5ccccc5)c34)cc2)cc1]

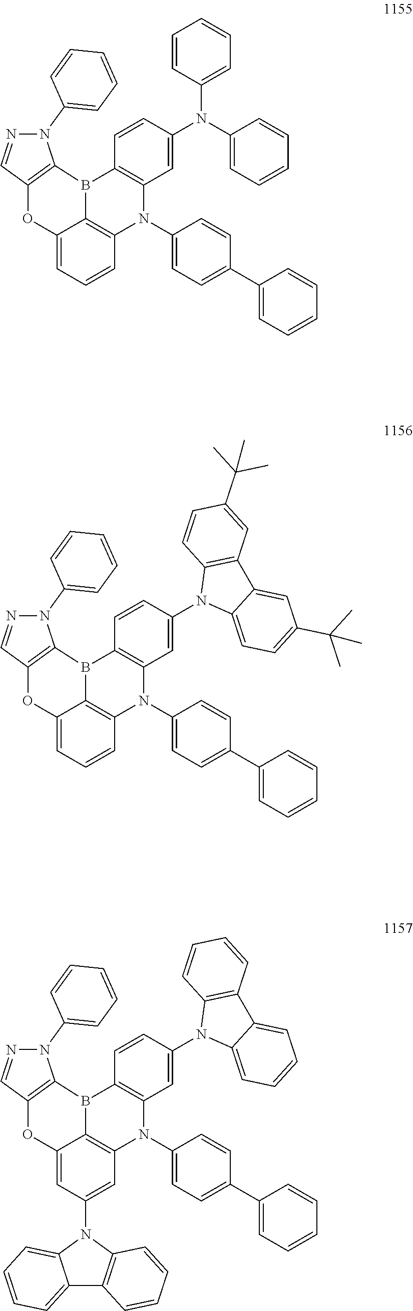 [SMILES: c1ccc(-c2ccc(N3c4cc(N(c5ccccc5)c5ccccc5)ccc4B4c5c(cccc53)Oc3cnn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(cnn1-c1ccccc1)O3 ; c1ccc(-c2ccc(N3c4cc(-n5c6ccccc6c6ccccc65)ccc4B4c5c(cc(-n6c7ccccc7c7ccccc76)cc53)Oc3cnn(-c5ccccc5)c34)cc2)cc1]

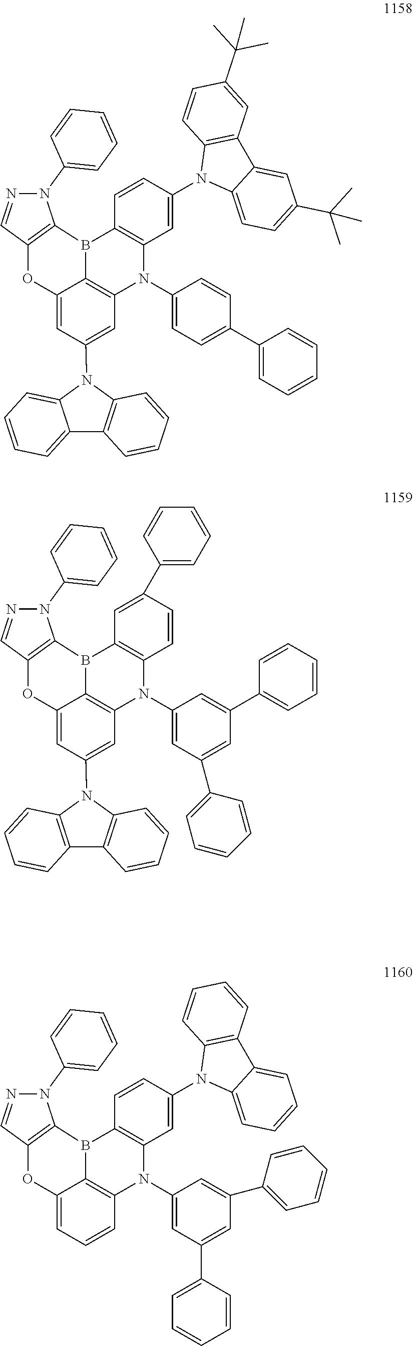 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(cnn1-c1ccccc1)O3 ; c1ccc(-c2cc(-c3ccccc3)cc(N3c4ccc(-c5ccccc5)cc4B4c5c(cc(-n6c7ccccc7c7ccccc76)cc53)Oc3cnn(-c5ccccc5)c34)c2)cc1 ; c1ccc(-c2cc(-c3ccccc3)cc(N3c4cc(-n5c6ccccc6c6ccccc65)ccc4B4c5c(cccc53)Oc3cnn(-c5ccccc5)c34)c2)cc1]

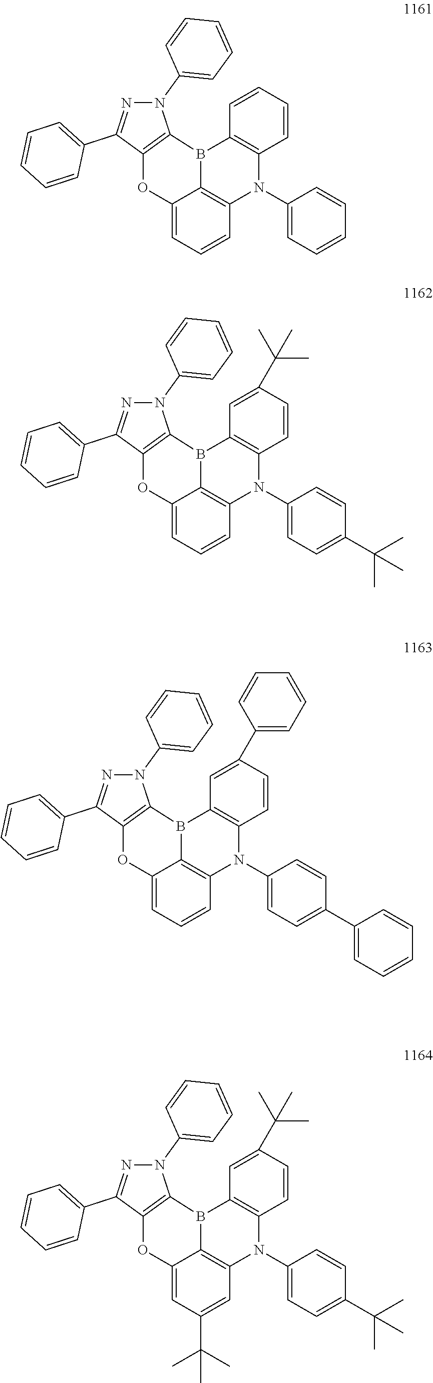 [SMILES: c1ccc(-c2nn(-c3ccccc3)c3c2Oc2cccc4c2B3c2ccccc2N4c2ccccc2)cc1 ; CC(C)(C)c1ccc(N2c3ccc(C(C)(C)C)cc3B3c4c(cccc42)Oc2c(-c4ccccc4)nn(-c4ccccc4)c23)cc1 ; c1ccc(-c2ccc(N3c4ccc(-c5ccccc5)cc4B4c5c(cccc53)Oc3c(-c5ccccc5)nn(-c5ccccc5)c34)cc2)cc1 ; CC(C)(C)c1ccc(N2c3ccc(C(C)(C)C)cc3B3c4c(cc(C(C)(C)C)cc42)Oc2c(-c4ccccc4)nn(-c4ccccc4)c23)cc1]

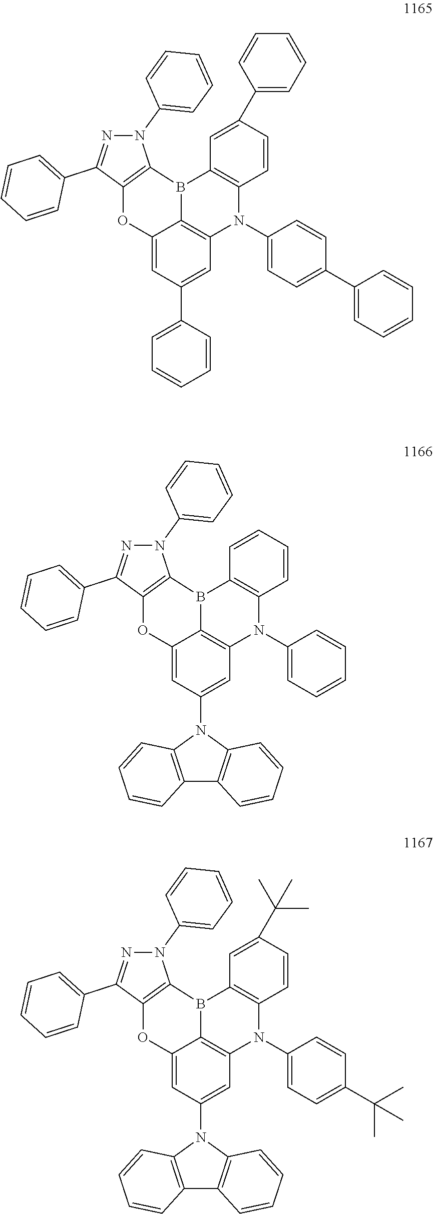 [SMILES: c1ccc(-c2ccc(N3c4ccc(-c5ccccc5)cc4B4c5c(cc(-c6ccccc6)cc53)Oc3c(-c5ccccc5)nn(-c5ccccc5)c34)cc2)cc1 ; c1ccc(-c2nn(-c3ccccc3)c3c2Oc2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4c(cc(-n5c6ccccc6c6ccccc65)cc42)Oc2c(-c4ccccc4)nn(-c4ccccc4)c23)cc1]

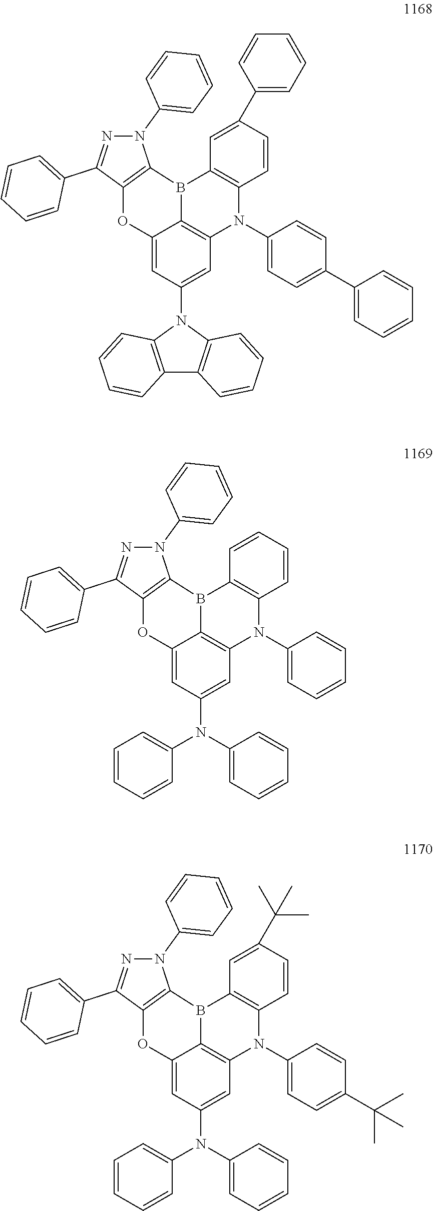 [SMILES: c1ccc(-c2ccc(N3c4ccc(-c5ccccc5)cc4B4c5c(cc(-n6c7ccccc7c7ccccc76)cc53)Oc3c(-c5ccccc5)nn(-c5ccccc5)c34)cc2)cc1 ; c1ccc(-c2nn(-c3ccccc3)c3c2Oc2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4c(cc(N(c5ccccc5)c5ccccc5)cc42)Oc2c(-c4ccccc4)nn(-c4ccccc4)c23)cc1]

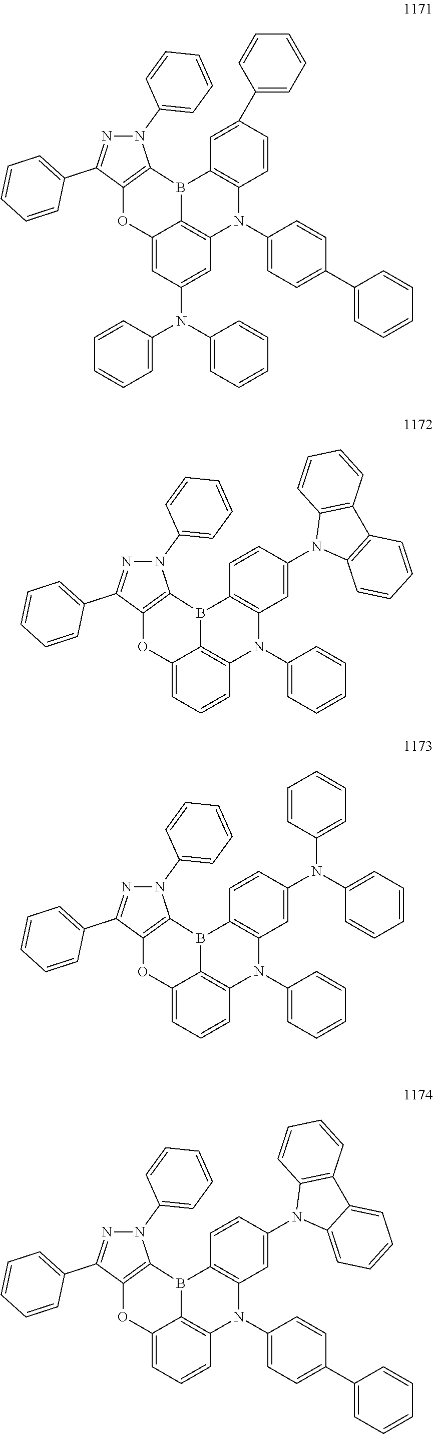 [SMILES: c1ccc(-c2ccc(N3c4ccc(-c5ccccc5)cc4B4c5c(cc(N(c6ccccc6)c6ccccc6)cc53)Oc3c(-c5ccccc5)nn(-c5ccccc5)c34)cc2)cc1 ; c1ccc(-c2nn(-c3ccccc3)c3c2Oc2cccc4c2B3c2ccc(-n3c5ccccc5c5ccccc53)cc2N4c2ccccc2)cc1 ; c1ccc(-c2nn(-c3ccccc3)c3c2Oc2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5c(cccc53)Oc3c(-c5ccccc5)nn(-c5ccccc5)c34)cc2)cc1]

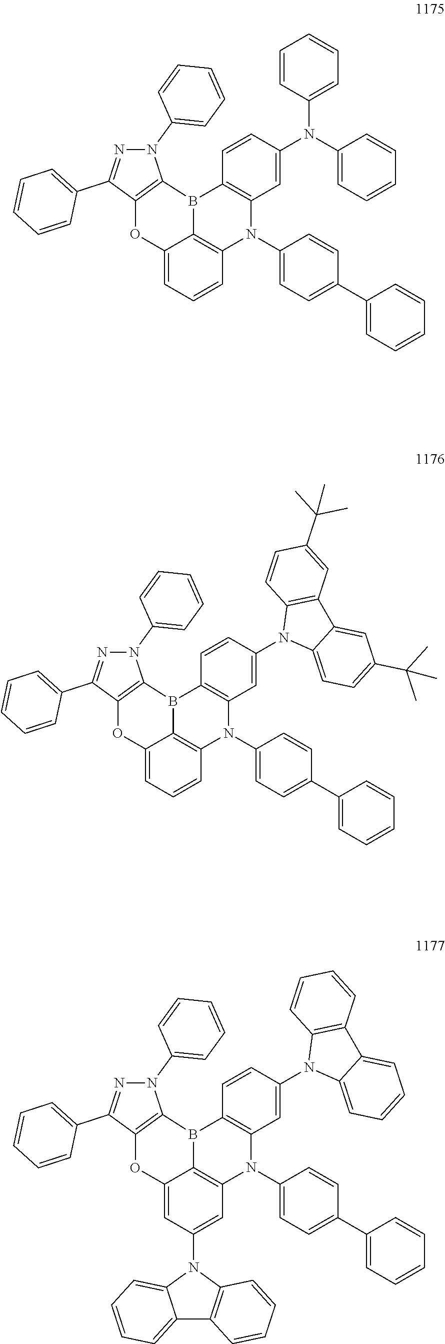 [SMILES: c1ccc(-c2ccc(N3c4cc(N(c5ccccc5)c5ccccc5)ccc4B4c5c(cccc53)Oc3c(-c5ccccc5)nn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(c(-c2ccccc2)nn1-c1ccccc1)O3 ; c1ccc(-c2ccc(N3c4cc(-n5c6ccccc6c6ccccc65)ccc4B4c5c(cc(-n6c7ccccc7c7ccccc76)cc53)Oc3c(-c5ccccc5)nn(-c5ccccc5)c34)cc2)cc1]

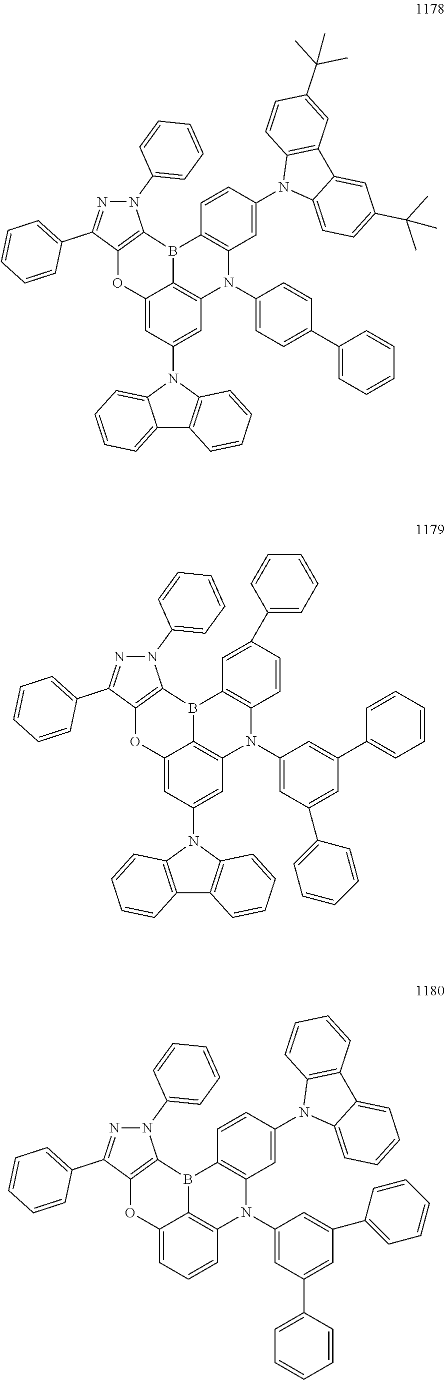 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(c(-c2ccccc2)nn1-c1ccccc1)O3 ; c1ccc(-c2cc(-c3ccccc3)cc(N3c4ccc(-c5ccccc5)cc4B4c5c(cc(-n6c7ccccc7c7ccccc76)cc53)Oc3c(-c5ccccc5)nn(-c5ccccc5)c34)c2)cc1 ; c1ccc(-c2cc(-c3ccccc3)cc(N3c4cc(-n5c6ccccc6c6ccccc65)ccc4B4c5c(cccc53)Oc3c(-c5ccccc5)nn(-c5ccccc5)c34)c2)cc1]

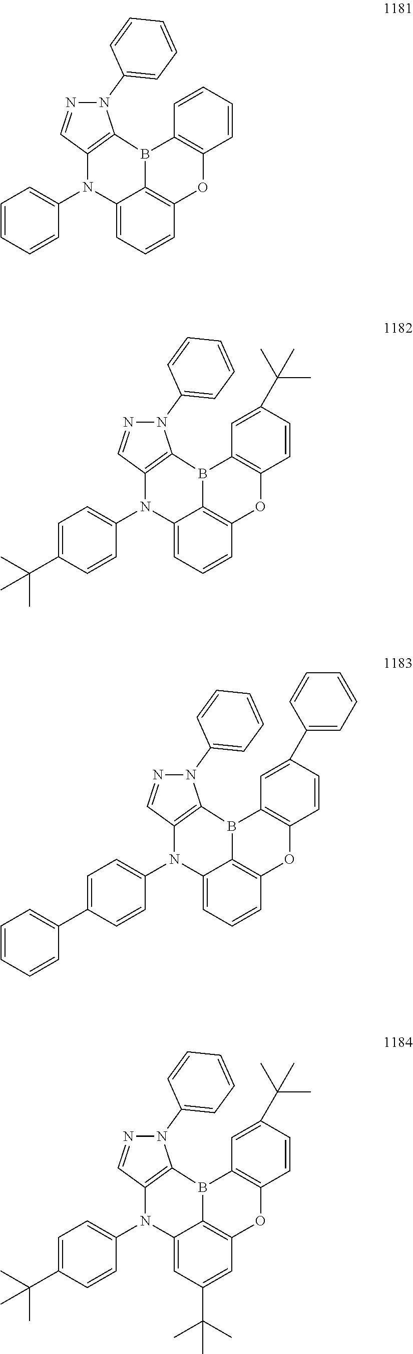

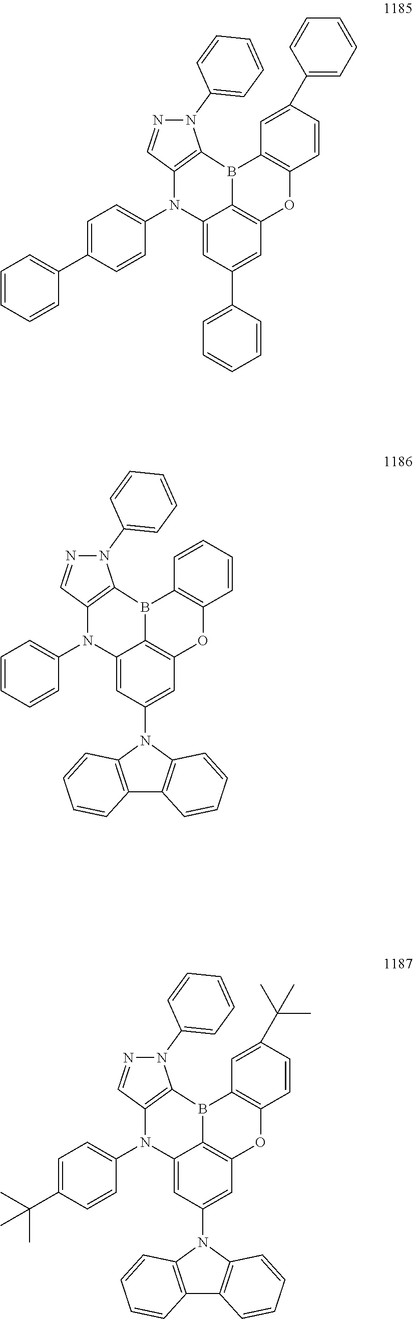 [SMILES: c1ccc(-c2ccc(N3c4cc(-c5ccccc5)cc5c4B(c4cc(-c6ccccc6)ccc4O5)c4c3cnn4-c3ccccc3)cc2)cc1 ; c1ccc(N2c3cc(-n4c5ccccc5c5ccccc54)cc4c3B(c3ccccc3O4)c3c2cnn3-c2ccccc2)cc1 ; CC(C)(C)c1ccc(N2c3cc(-n4c5ccccc5c5ccccc54)cc4c3B(c3cc(C(C)(C)C)ccc3O4)c3c2cnn3-c2ccccc2)cc1]

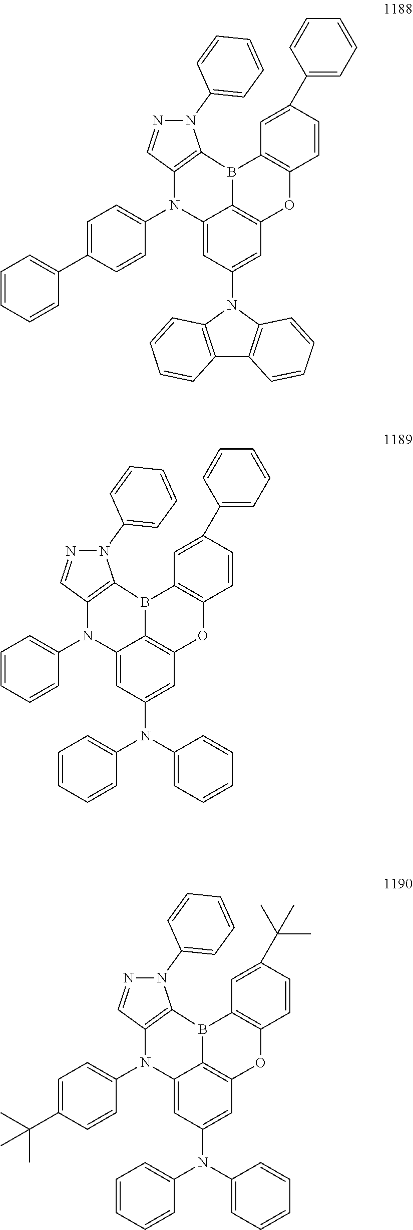 [SMILES: c1ccc(-c2ccc(N3c4cc(-n5c6ccccc6c6ccccc65)cc5c4B(c4cc(-c6ccccc6)ccc4O5)c4c3cnn4-c3ccccc3)cc2)cc1 ; c1ccc(-c2ccc3c(c2)B2c4c(cc(N(c5ccccc5)c5ccccc5)cc4N(c4ccccc4)c4cnn(-c5ccccc5)c42)O3)cc1 ; CC(C)(C)c1ccc(N2c3cc(N(c4ccccc4)c4ccccc4)cc4c3B(c3cc(C(C)(C)C)ccc3O4)c3c2cnn3-c2ccccc2)cc1]

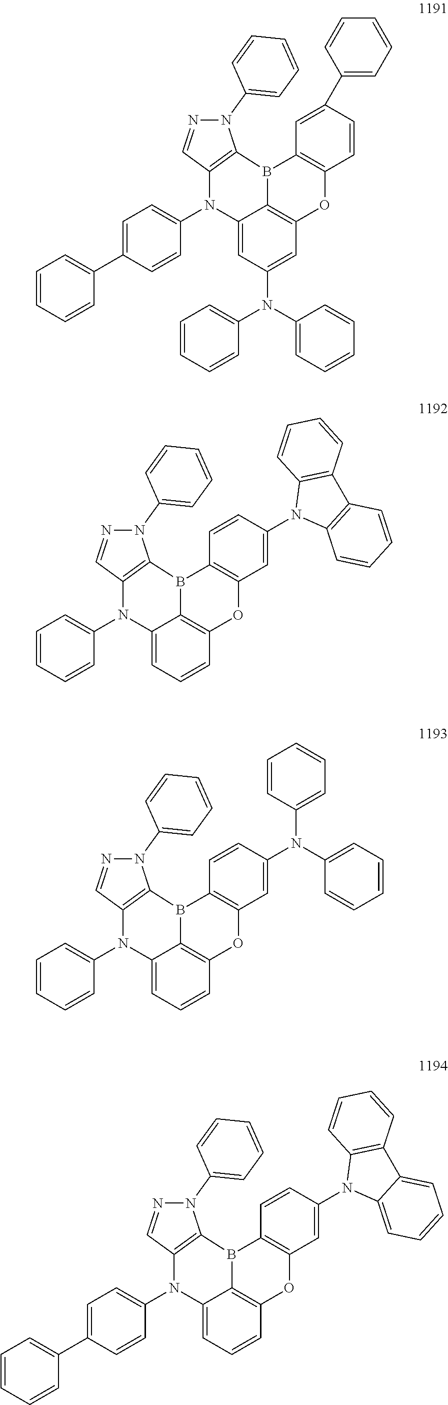 [SMILES: c1ccc(-c2ccc(N3c4cc(N(c5ccccc5)c5ccccc5)cc5c4B(c4cc(-c6ccccc6)ccc4O5)c4c3cnn4-c3ccccc3)cc2)cc1 ; c1ccc(N2c3cccc4c3B(c3ccc(-n5c6ccccc6c6ccccc65)cc3O4)c3c2cnn3-c2ccccc2)cc1 ; c1ccc(N(c2ccccc2)c2ccc3c(c2)Oc2cccc4c2B3c2c(cnn2-c2ccccc2)N4c2ccccc2)cc1 ; c1ccc(-c2ccc(N3c4cccc5c4B(c4ccc(-n6c7ccccc7c7ccccc76)cc4O5)c4c3cnn4-c3ccccc3)cc2)cc1]

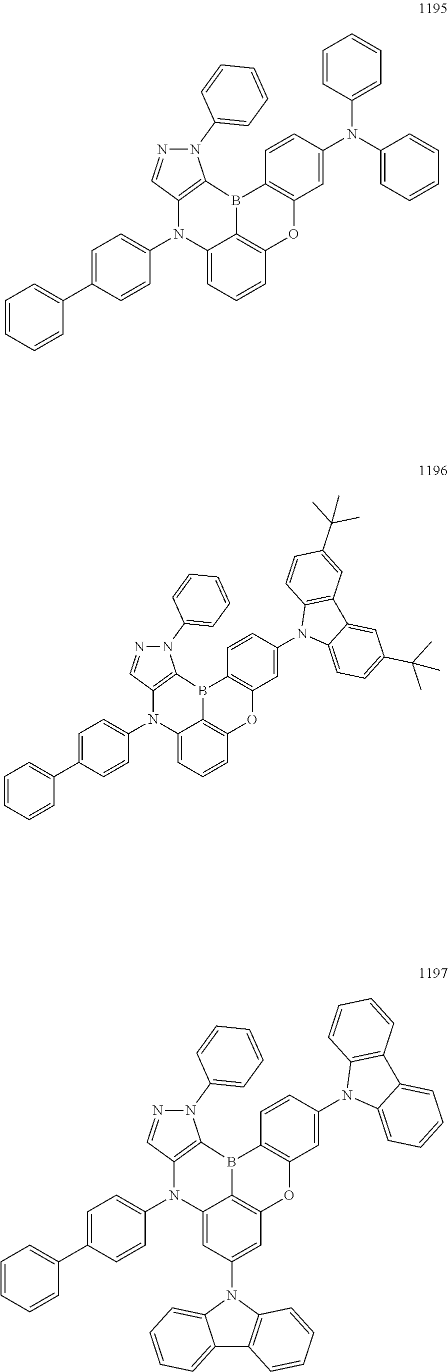 [SMILES: c1ccc(-c2ccc(N3c4cccc5c4B(c4ccc(N(c6ccccc6)c6ccccc6)cc4O5)c4c3cnn4-c3ccccc3)cc2)cc1 ; CC(C)(C)c1ccc2c(c1)c1cc(C(C)(C)C)ccc1n2-c1ccc2c(c1)Oc1cccc3c1B2c1c(cnn1-c1ccccc1)N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4c3cnn4-c3ccccc3)cc2)cc1]

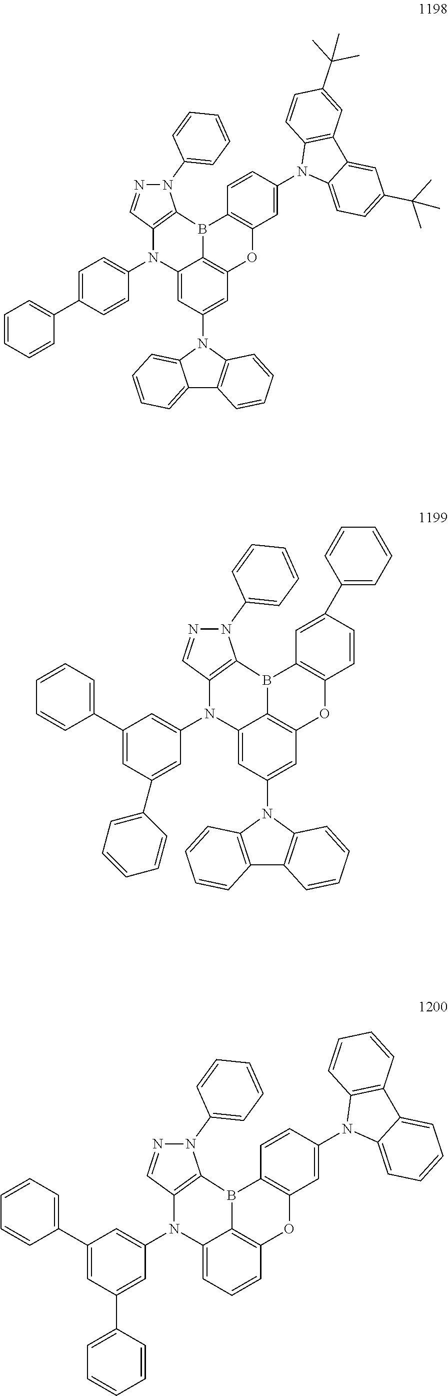 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(cnn1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3cnn4-c3ccccc3)c2)cc1 ; c1ccc(-c2cc(-c3ccccc3)cc(N3c4cccc5c4B(c4ccc(-n6c7ccccc7c7ccccc76)cc4O5)c4c3cnn4-c3ccccc3)c2)cc1]

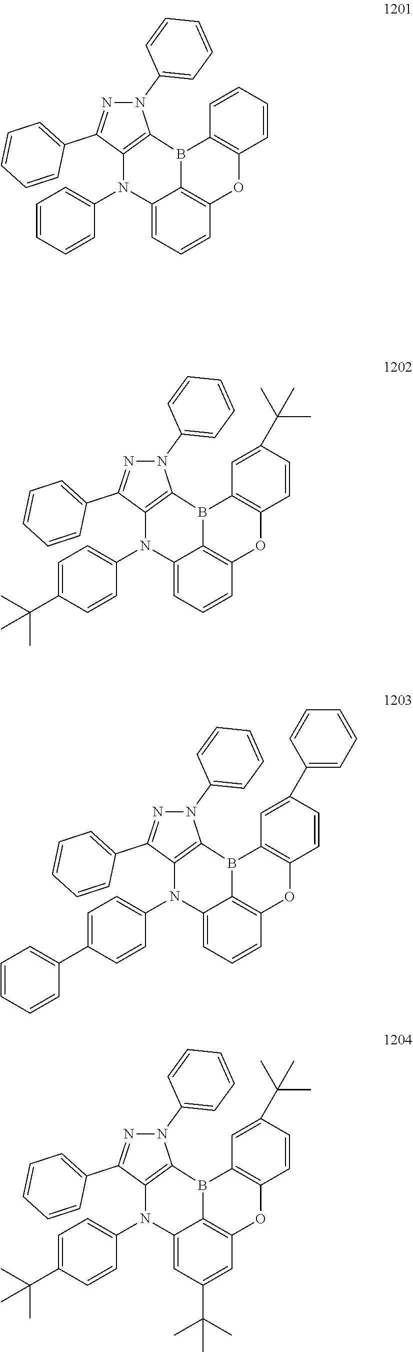

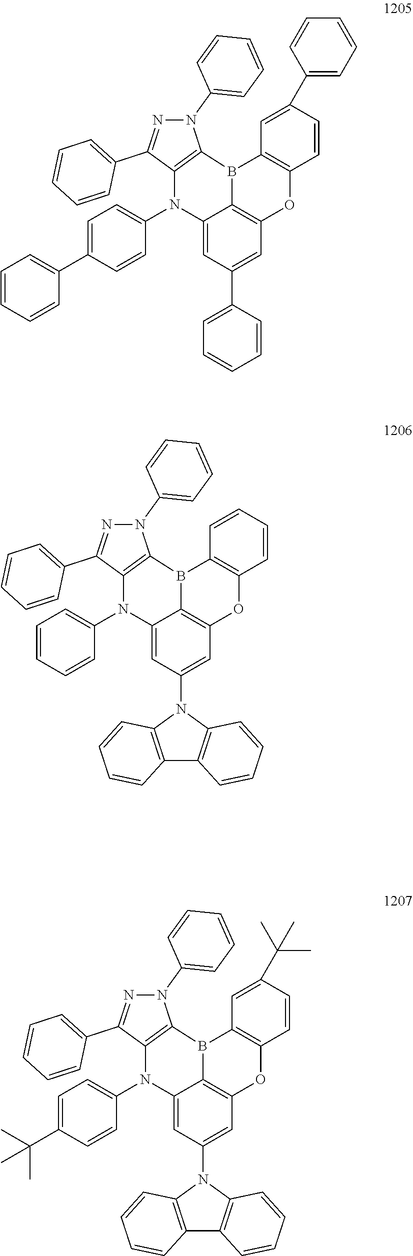 [SMILES: c1ccc(-c2ccc(N3c4cc(-c5ccccc5)cc5c4B(c4cc(-c6ccccc6)ccc4O5)c4c3c(-c3ccccc3)nn4-c3ccccc3)cc2)cc1 ; c1ccc(-c2nn(-c3ccccc3)c3c2N(c2ccccc2)c2cc(-n4c5ccccc5c5ccccc54)cc4c2B3c2ccccc2O4)cc1 ; CC(C)(C)c1ccc(N2c3cc(-n4c5ccccc5c5ccccc54)cc4c3B(c3cc(C(C)(C)C)ccc3O4)c3c2c(-c2ccccc2)nn3-c2ccccc2)cc1]

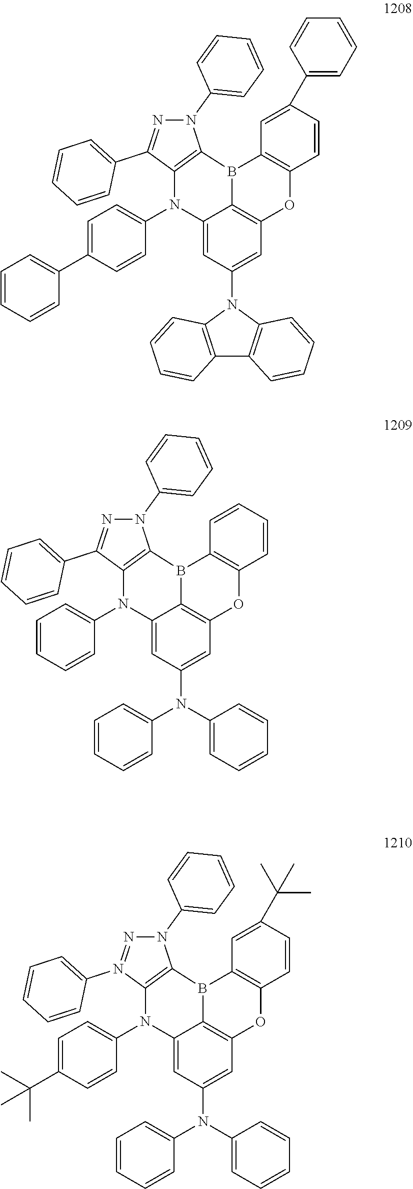

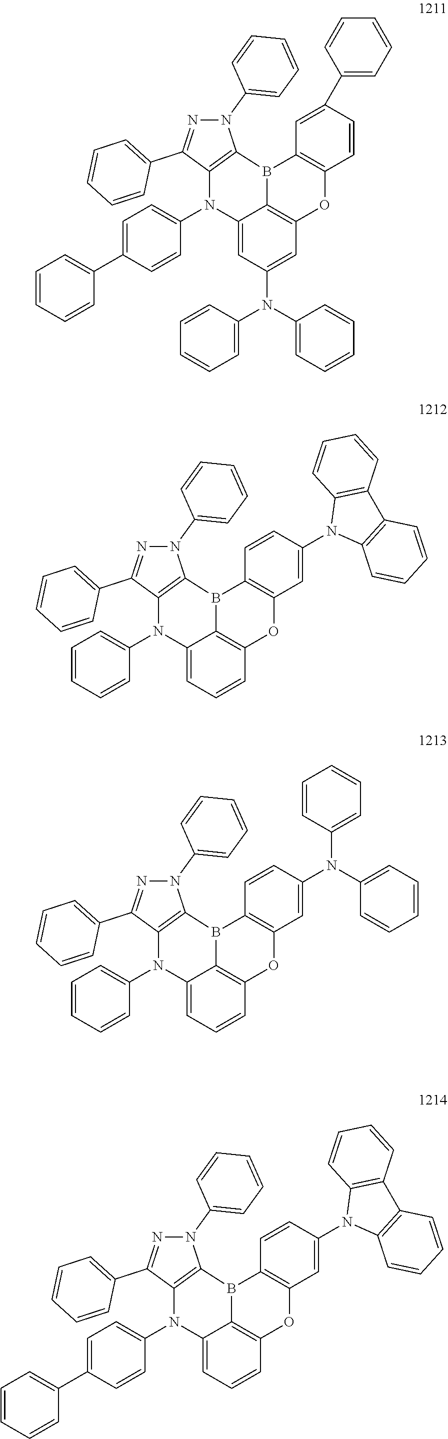 [SMILES: c1ccc(-c2ccc(N3c4cc(N(c5ccccc5)c5ccccc5)cc5c4B(c4cc(-c6ccccc6)ccc4O5)c4c3c(-c3ccccc3)nn4-c3ccccc3)cc2)cc1 ; c1ccc(-c2nn(-c3ccccc3)c3c2N(c2ccccc2)c2cccc4c2B3c2ccc(-n3c5ccccc5c5ccccc53)cc2O4)cc1 ; c1ccc(-c2nn(-c3ccccc3)c3c2N(c2ccccc2)c2cccc4c2B3c2ccc(N(c3ccccc3)c3ccccc3)cc2O4)cc1 ; c1ccc(-c2ccc(N3c4cccc5c4B(c4ccc(-n6c7ccccc7c7ccccc76)cc4O5)c4c3c(-c3ccccc3)nn4-c3ccccc3)cc2)cc1]

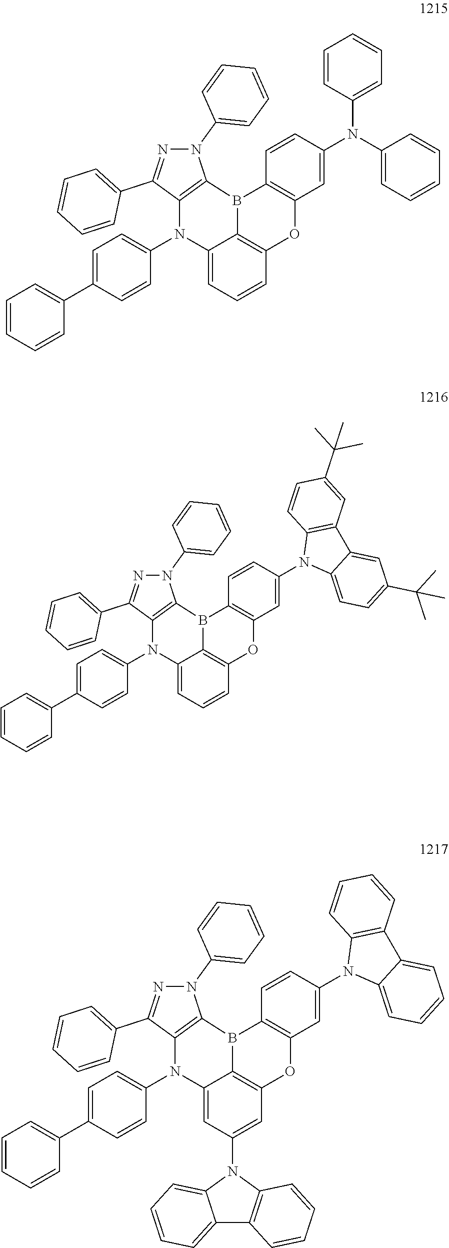 [SMILES: c1ccc(-c2ccc(N3c4cccc5c4B(c4ccc(N(c6ccccc6)c6ccccc6)cc4O5)c4c3c(-c3ccccc3)nn4-c3ccccc3)cc2)cc1 ; CC(C)(C)c1ccc2c(c1)c1cc(C(C)(C)C)ccc1n2-c1ccc2c(c1)Oc1cccc3c1B2c1c(c(-c2ccccc2)nn1-c1ccccc1)N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4c3c(-c3ccccc3)nn4-c3ccccc3)cc2)cc1]

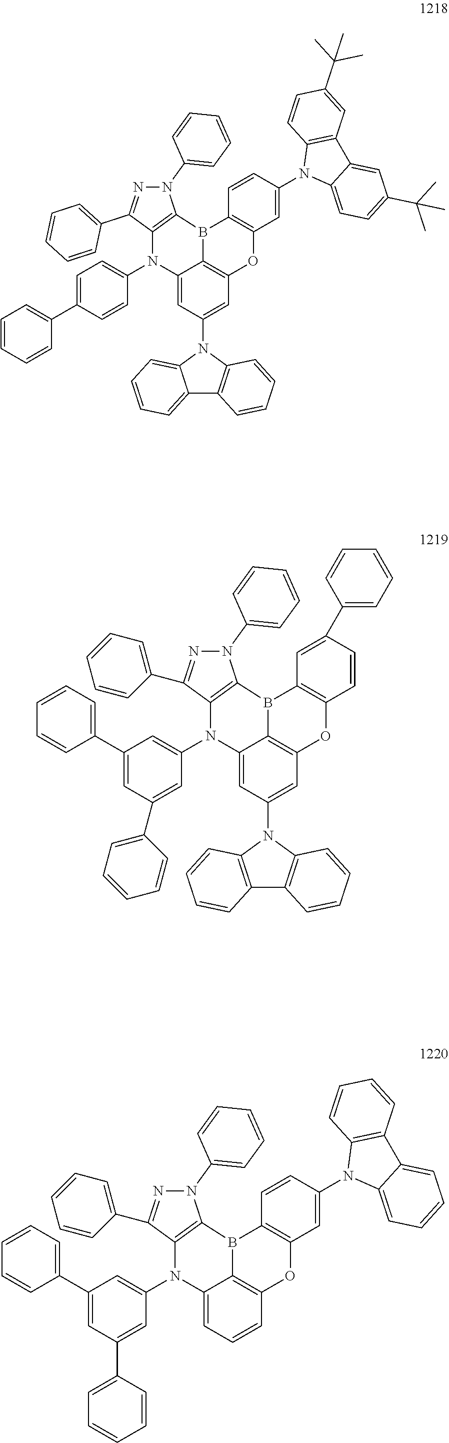 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(c(-c2ccccc2)nn1-c1ccccc1)N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c3c(-c3ccccc3)nn4-c3ccccc3)c2)cc1 ; c1ccc(-c2cc(-c3ccccc3)cc(N3c4cccc5c4B(c4ccc(-n6c7ccccc7c7ccccc76)cc4O5)c4c3c(-c3ccccc3)nn4-c3ccccc3)c2)cc1]

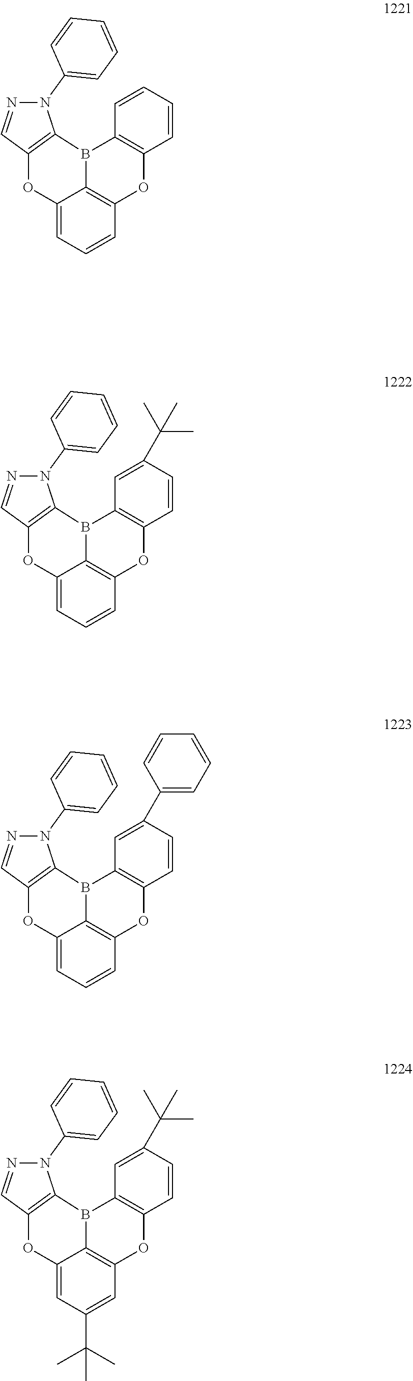 [SMILES: c1ccc(-n2ncc3c2B2c4ccccc4Oc4cccc(c42)O3)cc1 ; CC(C)(C)c1ccc2c(c1)B1c3c(cccc3Oc3cnn(-c4ccccc4)c31)O2 ; c1ccc(-c2ccc3c(c2)B2c4c(cccc4Oc4cnn(-c5ccccc5)c42)O3)cc1 ; CC(C)(C)c1cc2c3c(c1)Oc1cnn(-c4ccccc4)c1B3c1cc(C(C)(C)C)ccc1O2]

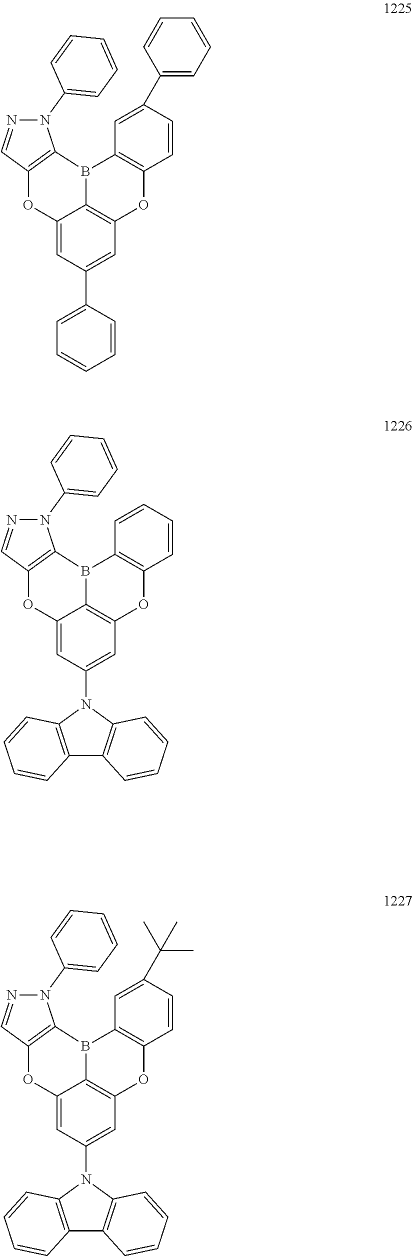 [SMILES: c1ccc(-c2cc3c4c(c2)Oc2cnn(-c5ccccc5)c2B4c2cc(-c4ccccc4)ccc2O3)cc1 ; c1ccc(-n2ncc3c2B2c4ccccc4Oc4cc(-n5c6ccccc6c6ccccc65)cc(c42)O3)cc1 ; CC(C)(C)c1ccc2c(c1)B1c3c(cc(-n4c5ccccc5c5ccccc54)cc3Oc3cnn(-c4ccccc4)c31)O2]

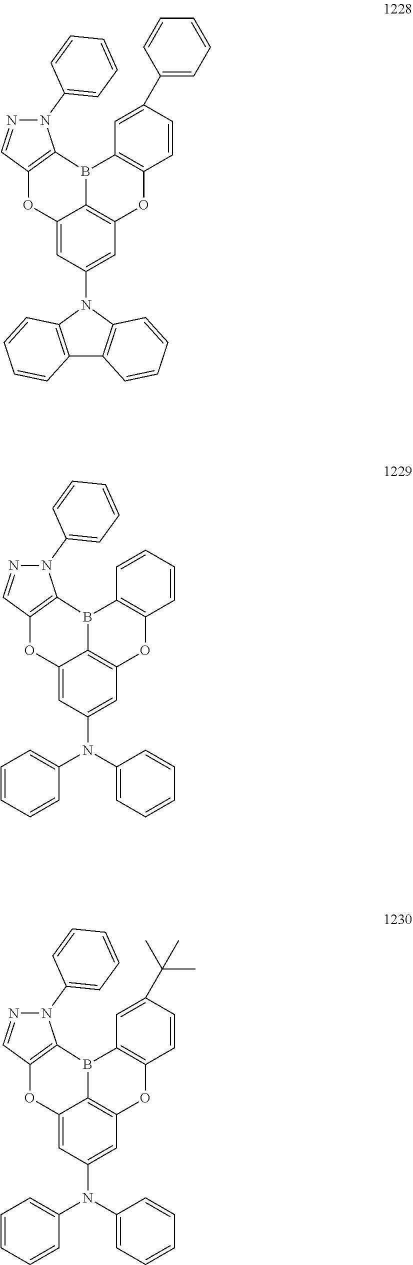 [SMILES: c1ccc(-c2ccc3c(c2)B2c4c(cc(-n5c6ccccc6c6ccccc65)cc4Oc4cnn(-c5ccccc5)c42)O3)cc1 ; c1ccc(N(c2ccccc2)c2cc3c4c(c2)Oc2cnn(-c5ccccc5)c2B4c2ccccc2O3)cc1 ; CC(C)(C)c1ccc2c(c1)B1c3c(cc(N(c4ccccc4)c4ccccc4)cc3Oc3cnn(-c4ccccc4)c31)O2]

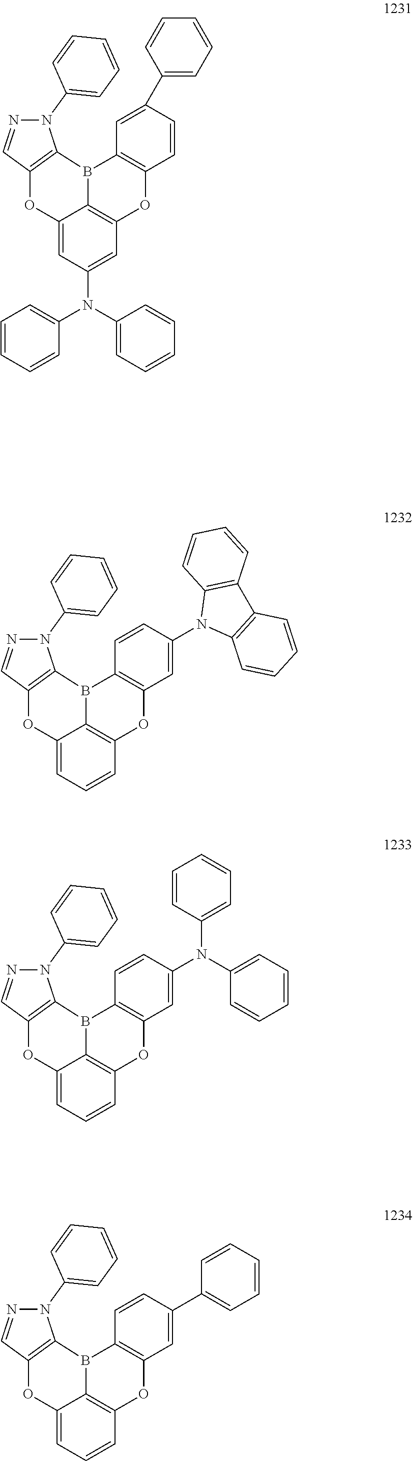 [SMILES: c1ccc(-c2ccc3c(c2)B2c4c(cc(N(c5ccccc5)c5ccccc5)cc4Oc4cnn(-c5ccccc5)c42)O3)cc1 ; c1ccc(-n2ncc3c2B2c4ccc(-n5c6ccccc6c6ccccc65)cc4Oc4cccc(c42)O3)cc1 ; c1ccc(N(c2ccccc2)c2ccc3c(c2)Oc2cccc4c2B3c2c(cnn2-c2ccccc2)O4)cc1 ; c1ccc(-c2ccc3c(c2)Oc2cccc4c2B3c2c(cnn2-c2ccccc2)O4)cc1]

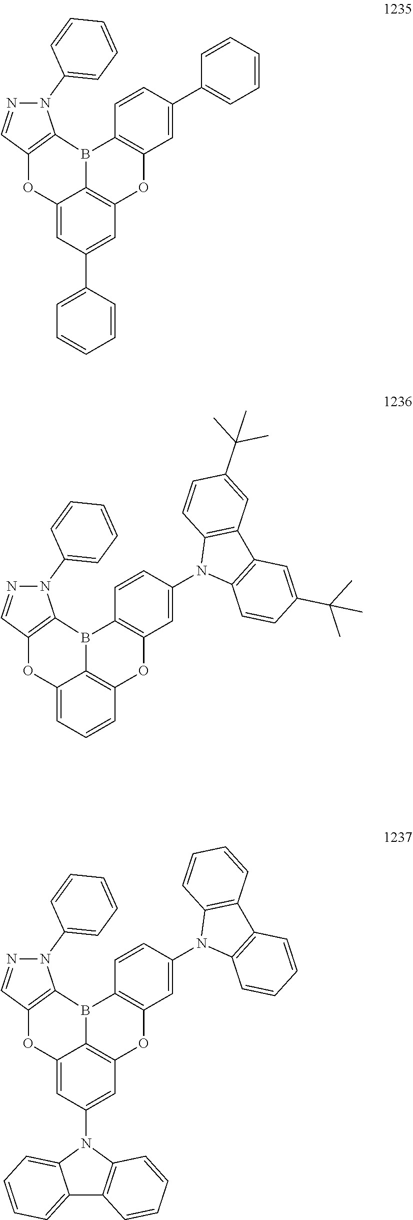 [SMILES: c1ccc(-c2ccc3c(c2)Oc2cc(-c4ccccc4)cc4c2B3c2c(cnn2-c2ccccc2)O4)cc1 ; CC(C)(C)c1ccc2c(c1)c1cc(C(C)(C)C)ccc1n2-c1ccc2c(c1)Oc1cccc3c1B2c1c(cnn1-c1ccccc1)O3 ; c1ccc(-n2ncc3c2B2c4ccc(-n5c6ccccc6c6ccccc65)cc4Oc4cc(-n5c6ccccc6c6ccccc65)cc(c42)O3)cc1]

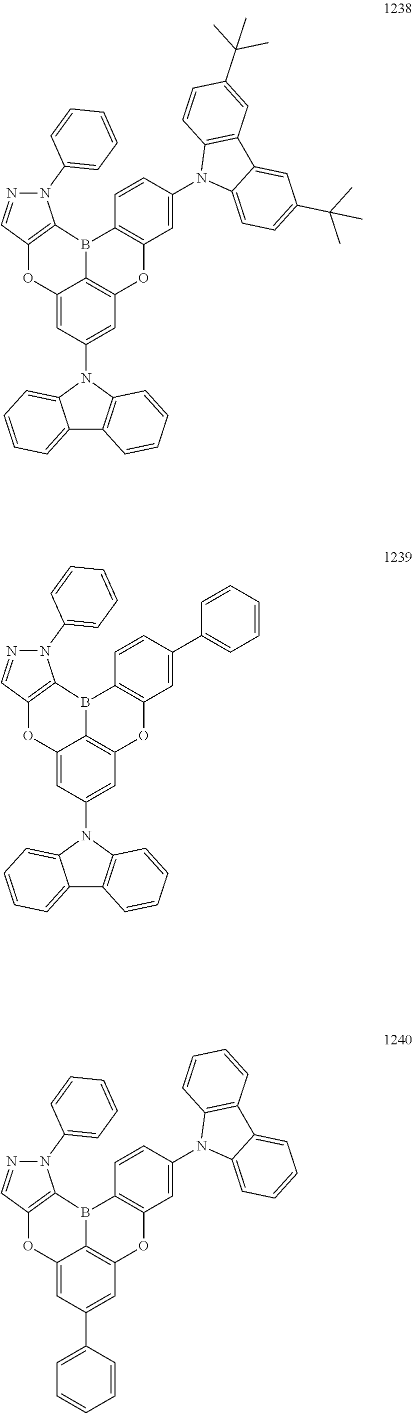 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(cnn1-c1ccccc1)O3 ; c1ccc(-c2ccc3c(c2)Oc2cc(-n4c5ccccc5c5ccccc54)cc4c2B3c2c(cnn2-c2ccccc2)O4)cc1 ; c1ccc(-c2cc3c4c(c2)Oc2cnn(-c5ccccc5)c2B4c2ccc(-n4c5ccccc5c5ccccc54)cc2O3)cc1]

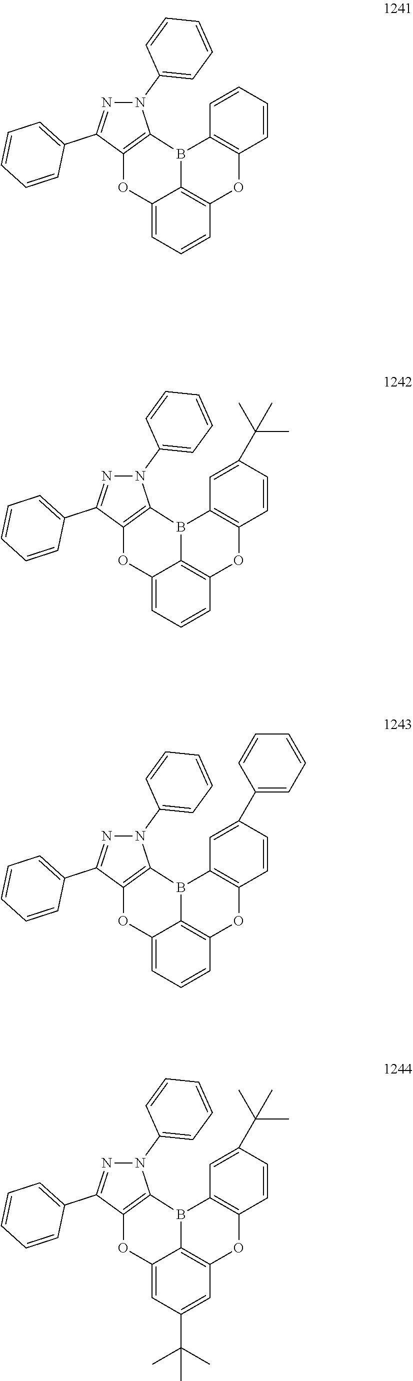 [SMILES: c1ccc(-c2nn(-c3ccccc3)c3c2Oc2cccc4c2B3c2ccccc2O4)cc1 ; CC(C)(C)c1ccc2c(c1)B1c3c(cccc3Oc3c(-c4ccccc4)nn(-c4ccccc4)c31)O2 ; c1ccc(-c2ccc3c(c2)B2c4c(cccc4Oc4c(-c5ccccc5)nn(-c5ccccc5)c42)O3)cc1 ; CC(C)(C)c1cc2c3c(c1)Oc1c(-c4ccccc4)nn(-c4ccccc4)c1B3c1cc(C(C)(C)C)ccc1O2]

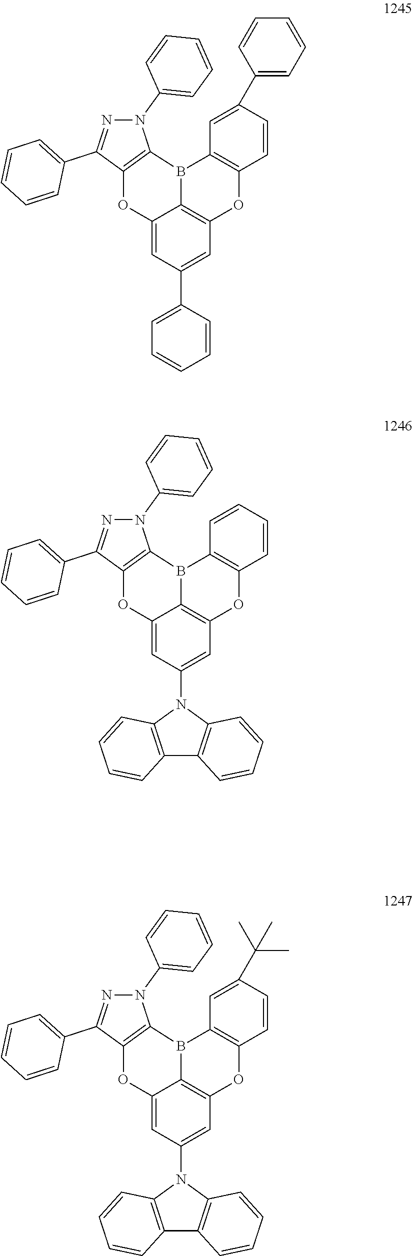 [SMILES: c1ccc(-c2cc3c4c(c2)Oc2c(-c5ccccc5)nn(-c5ccccc5)c2B4c2cc(-c4ccccc4)ccc2O3)cc1 ; c1ccc(-c2nn(-c3ccccc3)c3c2Oc2cc(-n4c5ccccc5c5ccccc54)cc4c2B3c2ccccc2O4)cc1 ; CC(C)(C)c1ccc2c(c1)B1c3c(cc(-n4c5ccccc5c5ccccc54)cc3Oc3c(-c4ccccc4)nn(-c4ccccc4)c31)O2]

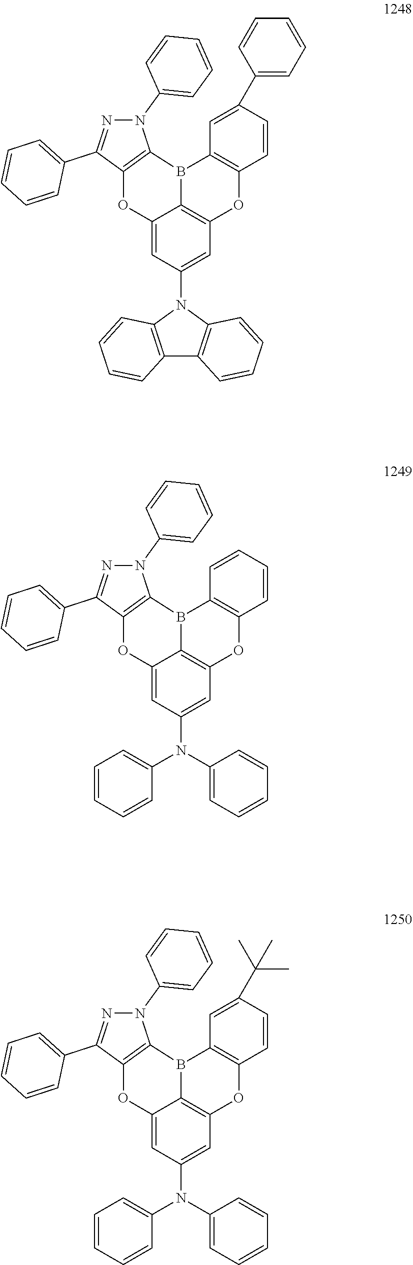 [SMILES: c1ccc(-c2ccc3c(c2)B2c4c(cc(-n5c6ccccc6c6ccccc65)cc4Oc4c(-c5ccccc5)nn(-c5ccccc5)c42)O3)cc1 ; c1ccc(-c2nn(-c3ccccc3)c3c2Oc2cc(N(c4ccccc4)c4ccccc4)cc4c2B3c2ccccc2O4)cc1 ; CC(C)(C)c1ccc2c(c1)B1c3c(cc(N(c4ccccc4)c4ccccc4)cc3Oc3c(-c4ccccc4)nn(-c4ccccc4)c31)O2]

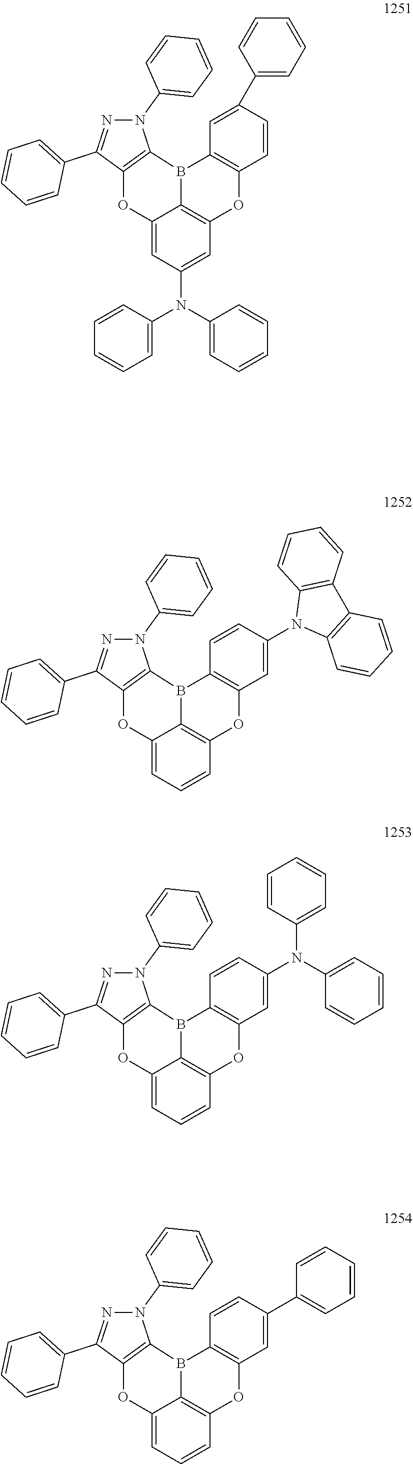 [SMILES: c1ccc(-c2ccc3c(c2)B2c4c(cc(N(c5ccccc5)c5ccccc5)cc4Oc4c(-c5ccccc5)nn(-c5ccccc5)c42)O3)cc1 ; c1ccc(-c2nn(-c3ccccc3)c3c2Oc2cccc4c2B3c2ccc(-n3c5ccccc5c5ccccc53)cc2O4)cc1 ; c1ccc(-c2nn(-c3ccccc3)c3c2Oc2cccc4c2B3c2ccc(N(c3ccccc3)c3ccccc3)cc2O4)cc1 ; c1ccc(-c2ccc3c(c2)Oc2cccc4c2B3c2c(c(-c3ccccc3)nn2-c2ccccc2)O4)cc1]

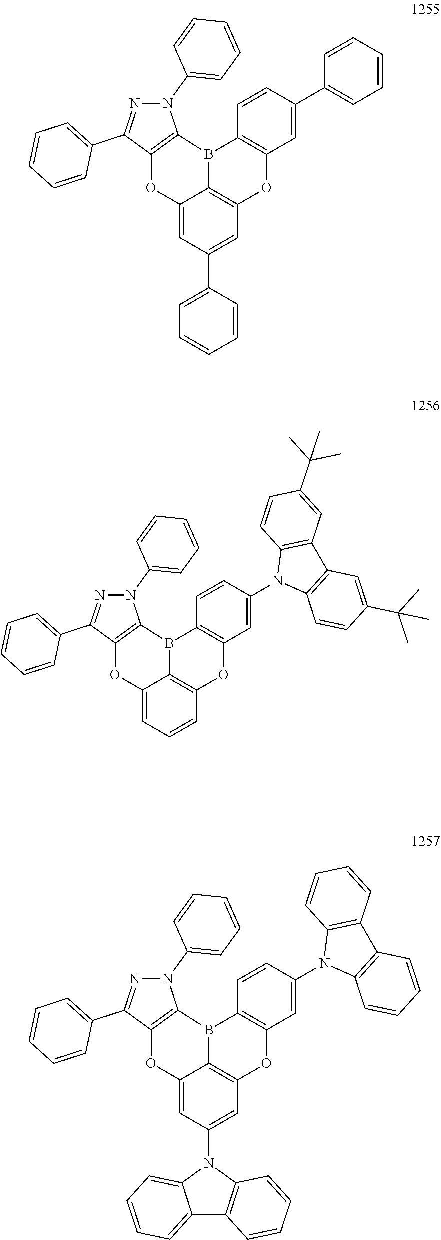 [SMILES: c1ccc(-c2ccc3c(c2)Oc2cc(-c4ccccc4)cc4c2B3c2c(c(-c3ccccc3)nn2-c2ccccc2)O4)cc1 ; CC(C)(C)c1ccc2c(c1)c1cc(C(C)(C)C)ccc1n2-c1ccc2c(c1)Oc1cccc3c1B2c1c(c(-c2ccccc2)nn1-c1ccccc1)O3 ; c1ccc(-c2nn(-c3ccccc3)c3c2Oc2cc(-n4c5ccccc5c5ccccc54)cc4c2B3c2ccc(-n3c5ccccc5c5ccccc53)cc2O4)cc1]

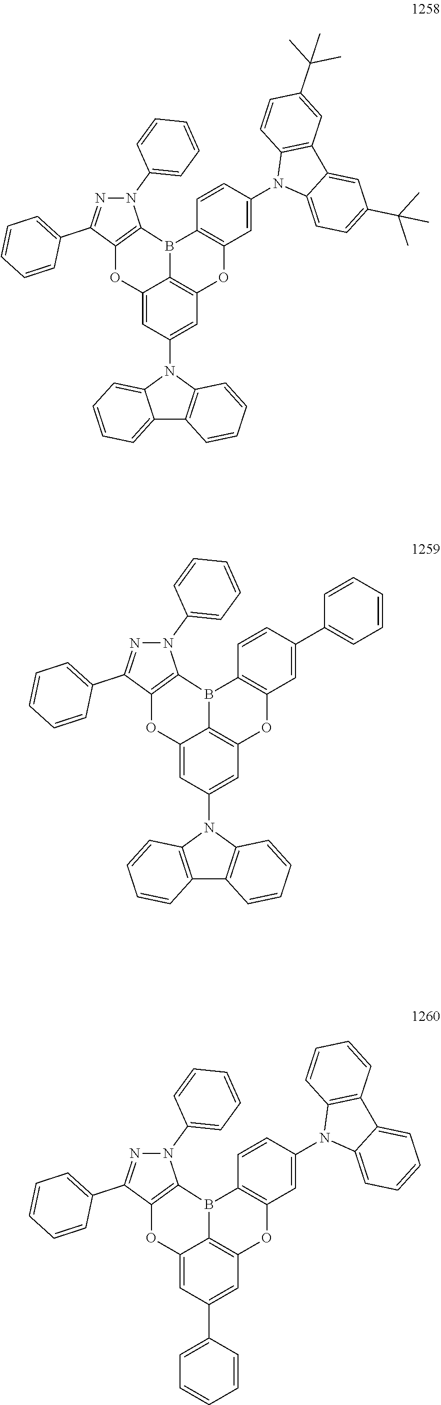 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(c(-c2ccccc2)nn1-c1ccccc1)O3 ; c1ccc(-c2ccc3c(c2)Oc2cc(-n4c5ccccc5c5ccccc54)cc4c2B3c2c(c(-c3ccccc3)nn2-c2ccccc2)O4)cc1 ; c1ccc(-c2cc3c4c(c2)Oc2c(-c5ccccc5)nn(-c5ccccc5)c2B4c2ccc(-n4c5ccccc5c5ccccc54)cc2O3)cc1]

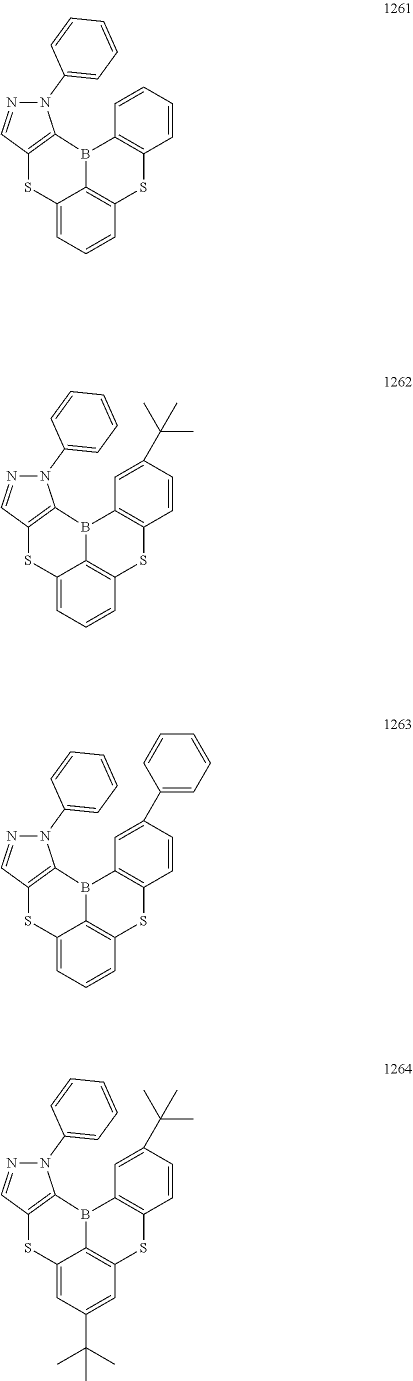 [SMILES: c1ccc(-n2ncc3c2B2c4ccccc4Sc4cccc(c42)S3)cc1 ; CC(C)(C)c1ccc2c(c1)B1c3c(cccc3Sc3cnn(-c4ccccc4)c31)S2 ; c1ccc(-c2ccc3c(c2)B2c4c(cccc4Sc4cnn(-c5ccccc5)c42)S3)cc1 ; CC(C)(C)c1cc2c3c(c1)Sc1cnn(-c4ccccc4)c1B3c1cc(C(C)(C)C)ccc1S2]

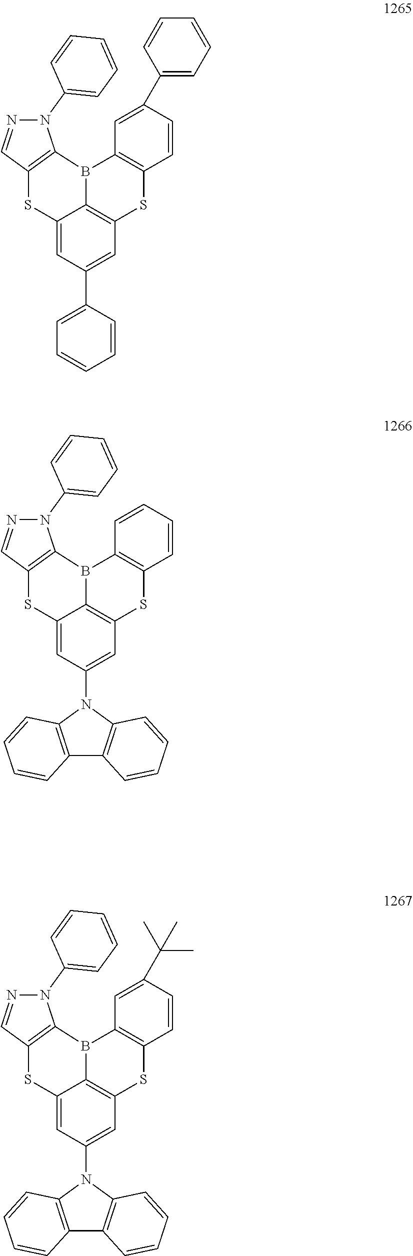 [SMILES: c1ccc(-c2cc3c4c(c2)Sc2cnn(-c5ccccc5)c2B4c2cc(-c4ccccc4)ccc2S3)cc1 ; c1ccc(-n2ncc3c2B2c4ccccc4Sc4cc(-n5c6ccccc6c6ccccc65)cc(c42)S3)cc1 ; CC(C)(C)c1ccc2c(c1)B1c3c(cc(-n4c5ccccc5c5ccccc54)cc3Sc3cnn(-c4ccccc4)c31)S2]

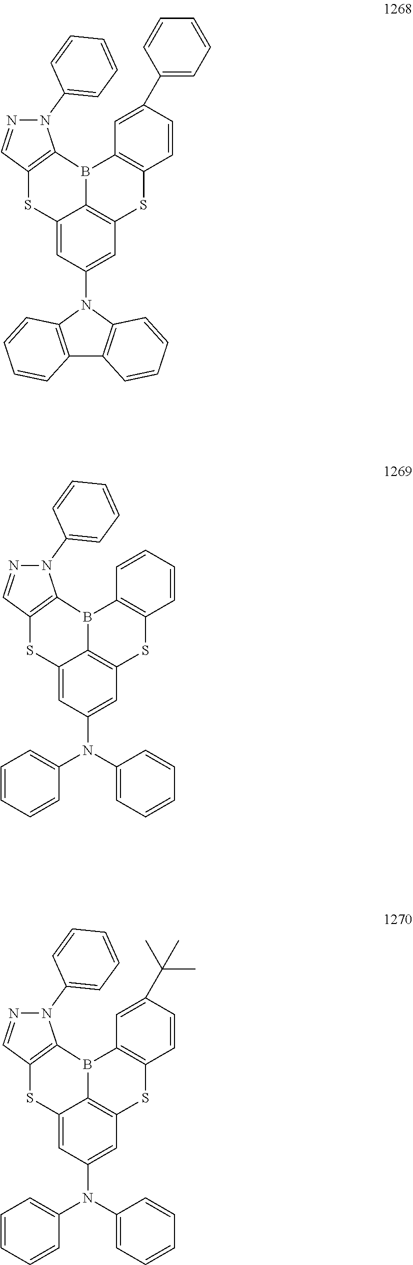 [SMILES: c1ccc(-c2ccc3c(c2)B2c4c(cc(-n5c6ccccc6c6ccccc65)cc4Sc4cnn(-c5ccccc5)c42)S3)cc1 ; c1ccc(N(c2ccccc2)c2cc3c4c(c2)Sc2cnn(-c5ccccc5)c2B4c2ccccc2S3)cc1 ; CC(C)(C)c1ccc2c(c1)B1c3c(cc(N(c4ccccc4)c4ccccc4)cc3Sc3cnn(-c4ccccc4)c31)S2]

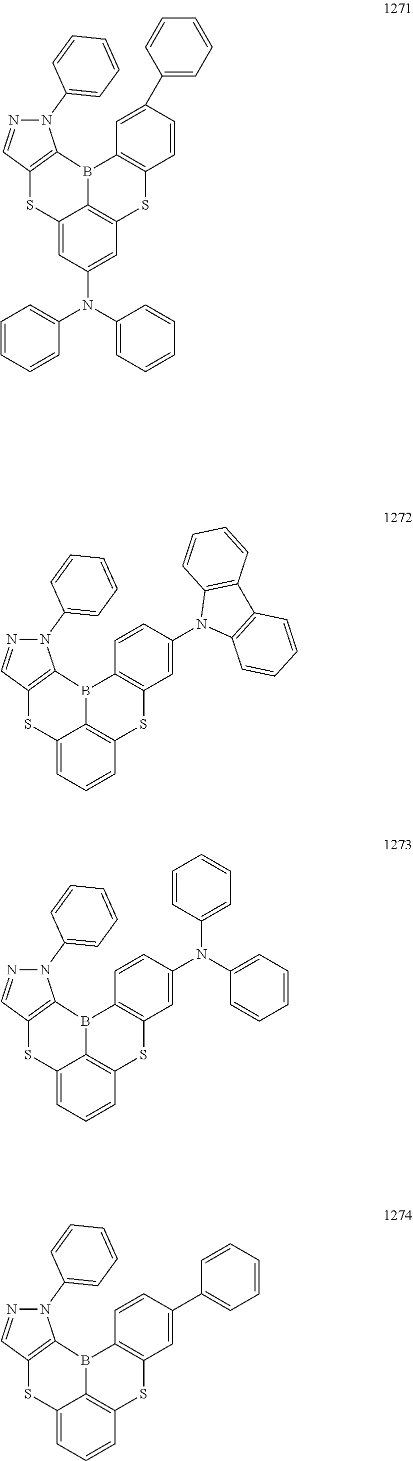 [SMILES: c1ccc(-c2ccc3c(c2)B2c4c(cc(N(c5ccccc5)c5ccccc5)cc4Sc4cnn(-c5ccccc5)c42)S3)cc1 ; c1ccc(-n2ncc3c2B2c4ccc(-n5c6ccccc6c6ccccc65)cc4Sc4cccc(c42)S3)cc1 ; c1ccc(N(c2ccccc2)c2ccc3c(c2)Sc2cccc4c2B3c2c(cnn2-c2ccccc2)S4)cc1 ; c1ccc(-c2ccc3c(c2)Sc2cccc4c2B3c2c(cnn2-c2ccccc2)S4)cc1]

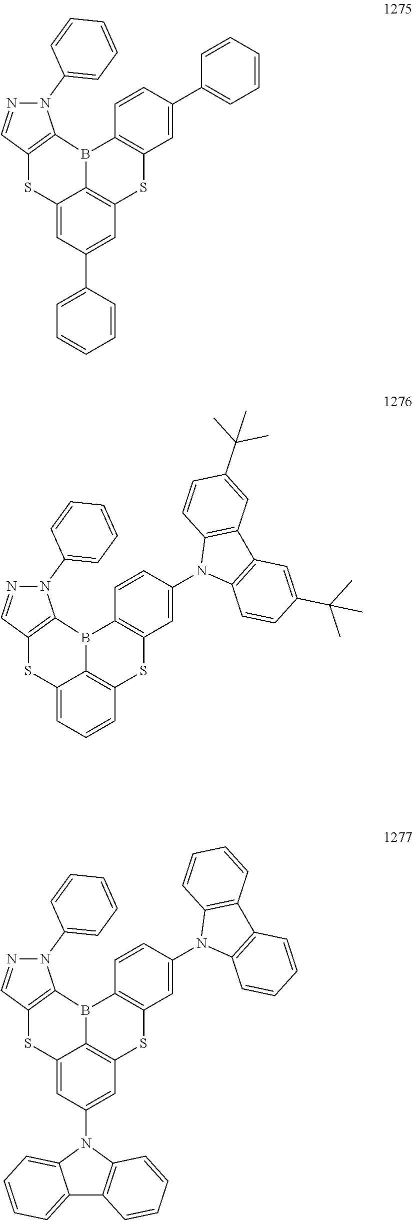 [SMILES: c1ccc(-c2ccc3c(c2)Sc2cc(-c4ccccc4)cc4c2B3c2c(cnn2-c2ccccc2)S4)cc1 ; CC(C)(C)c1ccc2c(c1)c1cc(C(C)(C)C)ccc1n2-c1ccc2c(c1)Sc1cccc3c1B2c1c(cnn1-c1ccccc1)S3 ; c1ccc(-n2ncc3c2B2c4ccc(-n5c6ccccc6c6ccccc65)cc4Sc4cc(-n5c6ccccc6c6ccccc65)cc(c42)S3)cc1]

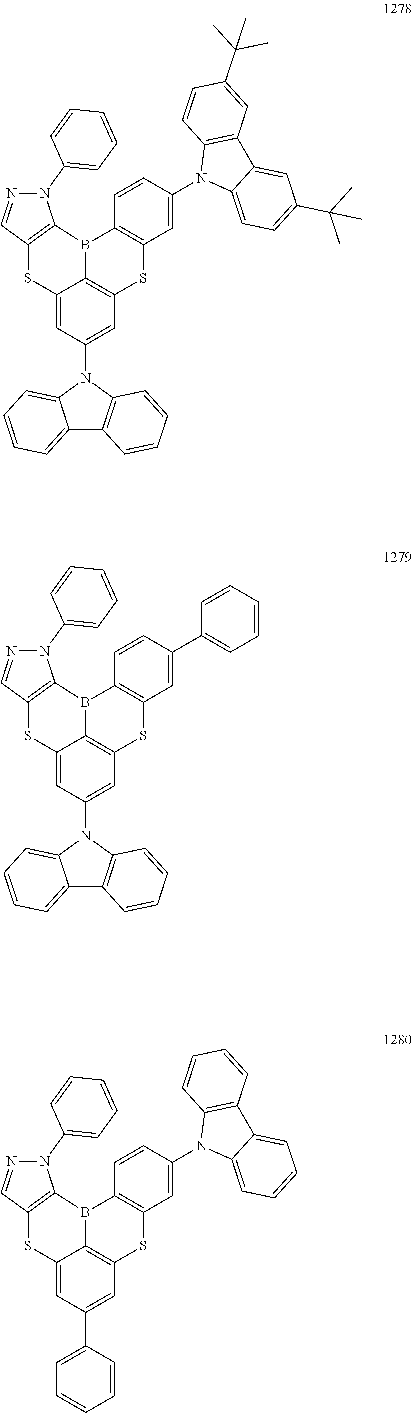 [SMILES: CC(C)(C)c1ccc2c(c1)c1cc(C(C)(C)C)ccc1n2-c1ccc2c(c1)Sc1cc(-n3c4ccccc4c4ccccc43)cc3c1B2c1c(cnn1-c1ccccc1)S3 ; c1ccc(-c2ccc3c(c2)Sc2cc(-n4c5ccccc5c5ccccc54)cc4c2B3c2c(cnn2-c2ccccc2)S4)cc1 ; c1ccc(-c2cc3c4c(c2)Sc2cnn(-c5ccccc5)c2B4c2ccc(-n4c5ccccc5c5ccccc54)cc2S3)cc1]

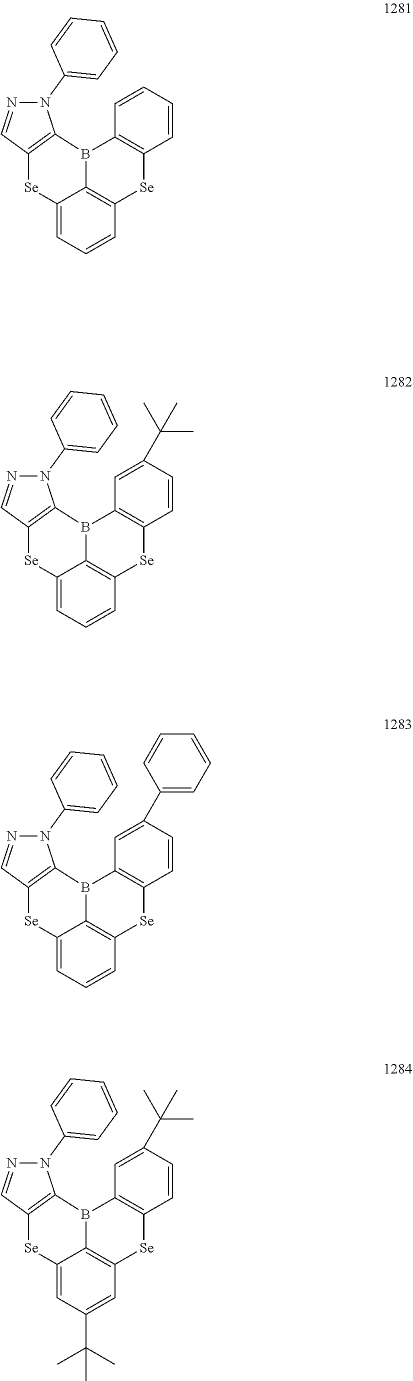 [SMILES: c1ccc(-n2ncc3c2B2c4ccccc4[Se]c4cccc(c42)[Se]3)cc1 ; CC(C)(C)c1ccc2c(c1)B1c3c(cccc3[Se]c3cnn(-c4ccccc4)c31)[Se]2 ; c1ccc(-c2ccc3c(c2)B2c4c(cccc4[Se]c4cnn(-c5ccccc5)c42)[Se]3)cc1 ; CC(C)(C)c1cc2c3c(c1)[Se]c1cnn(-c4ccccc4)c1B3c1cc(C(C)(C)C)ccc1[Se]2]

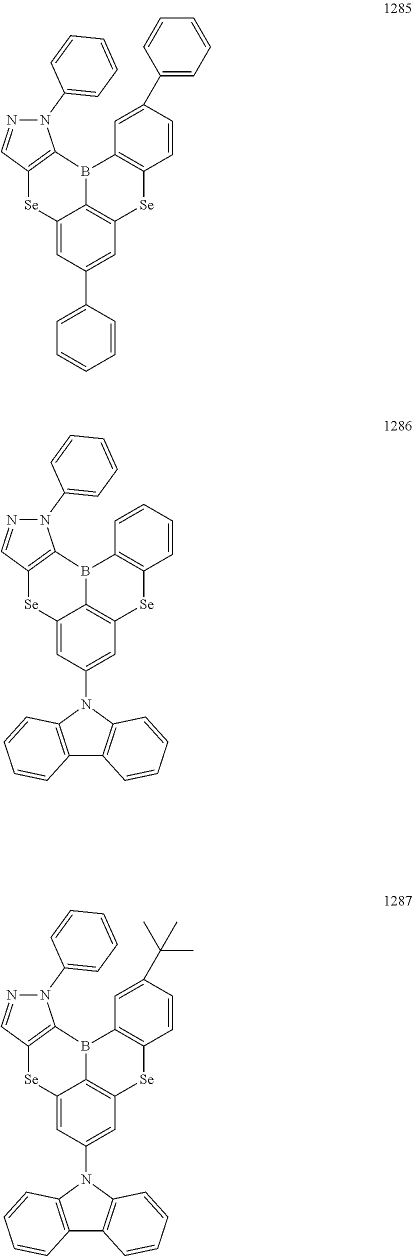 [SMILES: c1ccc(-c2cc3c4c(c2)[Se]c2cnn(-c5ccccc5)c2B4c2cc(-c4ccccc4)ccc2[Se]3)cc1 ; c1ccc(-n2ncc3c2B2c4ccccc4[Se]c4cc(-n5c6ccccc6c6ccccc65)cc(c42)[Se]3)cc1 ; CC(C)(C)c1ccc2c(c1)B1c3c(cc(-n4c5ccccc5c5ccccc54)cc3[Se]c3cnn(-c4ccccc4)c31)[Se]2]

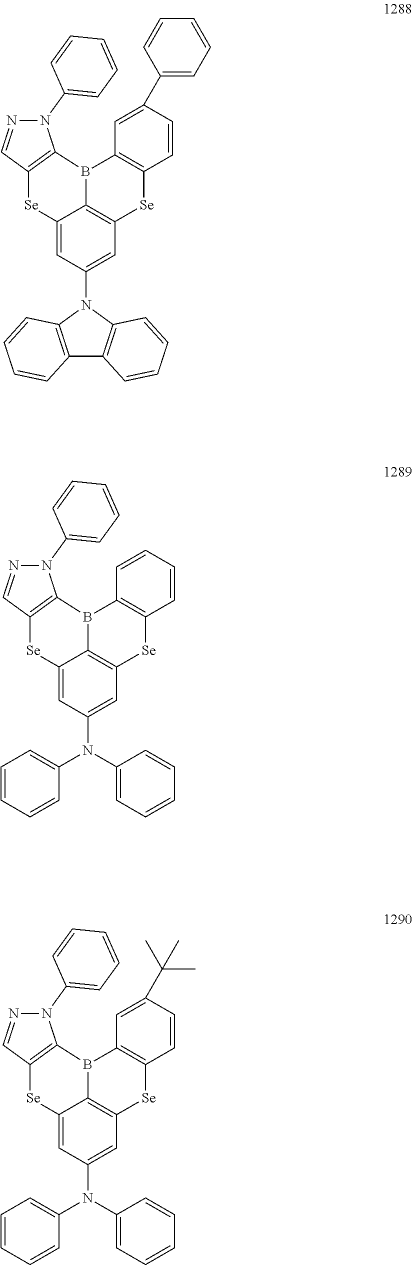 [SMILES: c1ccc(-c2ccc3c(c2)B2c4c(cc(-n5c6ccccc6c6ccccc65)cc4[Se]c4cnn(-c5ccccc5)c42)[Se]3)cc1 ; c1ccc(N(c2ccccc2)c2cc3c4c(c2)[Se]c2cnn(-c5ccccc5)c2B4c2ccccc2[Se]3)cc1 ; CC(C)(C)c1ccc2c(c1)B1c3c(cc(N(c4ccccc4)c4ccccc4)cc3[Se]c3cnn(-c4ccccc4)c31)[Se]2]

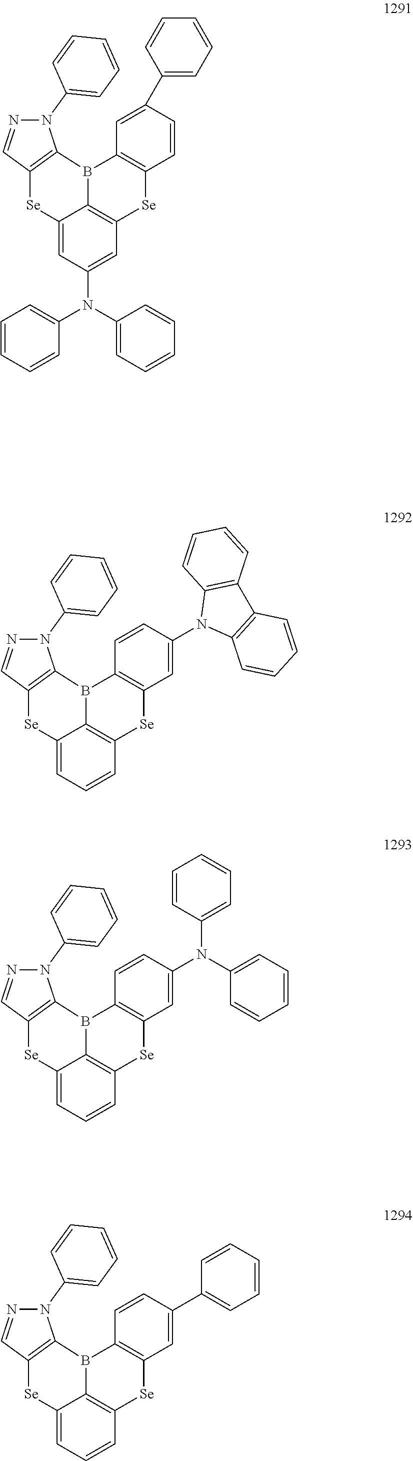 [SMILES: c1ccc(-c2ccc3c(c2)B2c4c(cc(N(c5ccccc5)c5ccccc5)cc4[Se]c4cnn(-c5ccccc5)c42)[Se]3)cc1 ; c1ccc(-n2ncc3c2B2c4ccc(-n5c6ccccc6c6ccccc65)cc4[Se]c4cccc(c42)[Se]3)cc1 ; c1ccc(N(c2ccccc2)c2ccc3c(c2)[Se]c2cccc4c2B3c2c(cnn2-c2ccccc2)[Se]4)cc1 ; c1ccc(-c2ccc3c(c2)[Se]c2cccc4c2B3c2c(cnn2-c2ccccc2)[Se]4)cc1]

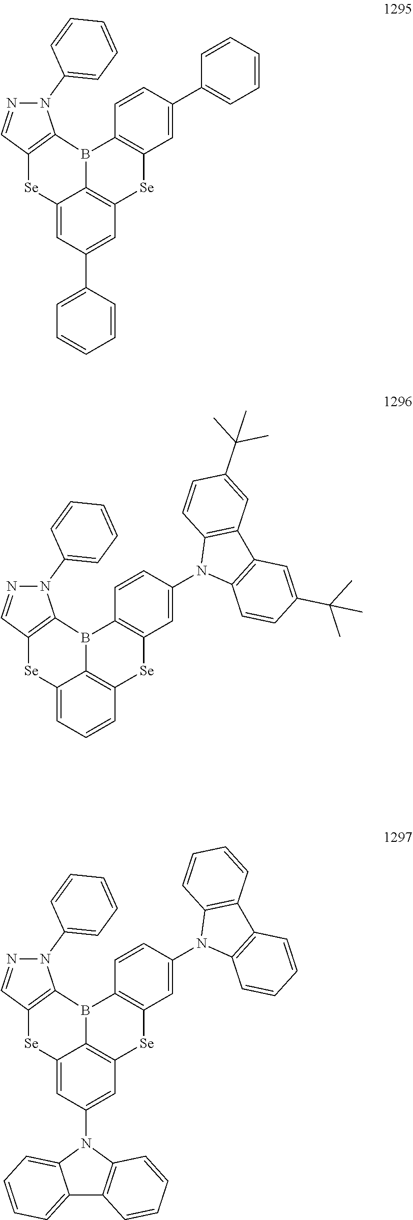 [SMILES: c1ccc(-c2ccc3c(c2)[Se]c2cc(-c4ccccc4)cc4c2B3c2c(cnn2-c2ccccc2)[Se]4)cc1 ; CC(C)(C)c1ccc2c(c1)c1cc(C(C)(C)C)ccc1n2-c1ccc2c(c1)[Se]c1cccc3c1B2c1c(cnn1-c1ccccc1)[Se]3 ; c1ccc(-n2ncc3c2B2c4ccc(-n5c6ccccc6c6ccccc65)cc4[Se]c4cc(-n5c6ccccc6c6ccccc65)cc(c42)[Se]3)cc1]

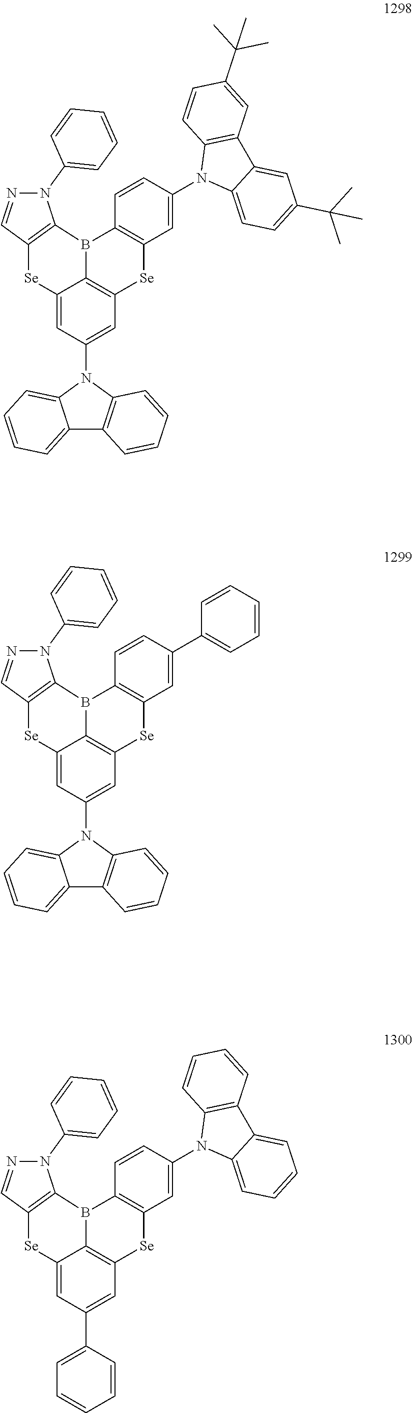 [SMILES: CC(C)(C)c1ccc2c(c1)c1cc(C(C)(C)C)ccc1n2-c1ccc2c(c1)[Se]c1cc(-n3c4ccccc4c4ccccc43)cc3c1B2c1c(cnn1-c1ccccc1)[Se]3 ; c1ccc(-c2ccc3c(c2)[Se]c2cc(-n4c5ccccc5c5ccccc54)cc4c2B3c2c(cnn2-c2ccccc2)[Se]4)cc1 ; c1ccc(-c2cc3c4c(c2)[Se]c2cnn(-c5ccccc5)c2B4c2ccc(-n4c5ccccc5c5ccccc54)cc2[Se]3)cc1]

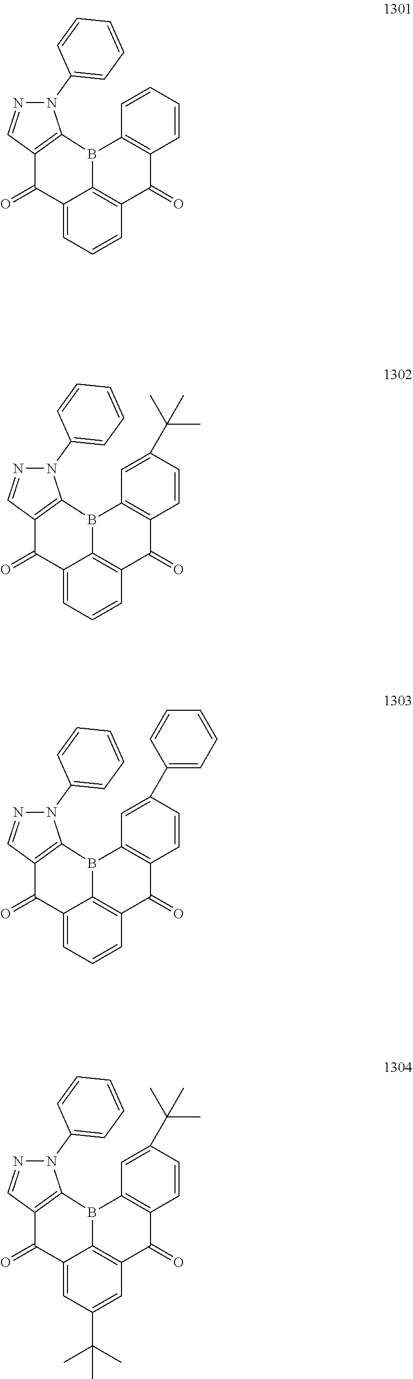 [SMILES: O=C1c2ccccc2B2c3c1cccc3C(=O)c1cnn(-c3ccccc3)c12 ; CC(C)(C)c1ccc2c(c1)B1c3c(cccc3C(=O)c3cnn(-c4ccccc4)c31)C2=O ; O=C1c2ccc(-c3ccccc3)cc2B2c3c1cccc3C(=O)c1cnn(-c3ccccc3)c12 ; CC(C)(C)c1ccc2c(c1)B1c3c(cc(C(C)(C)C)cc3C(=O)c3cnn(-c4ccccc4)c31)C2=O]

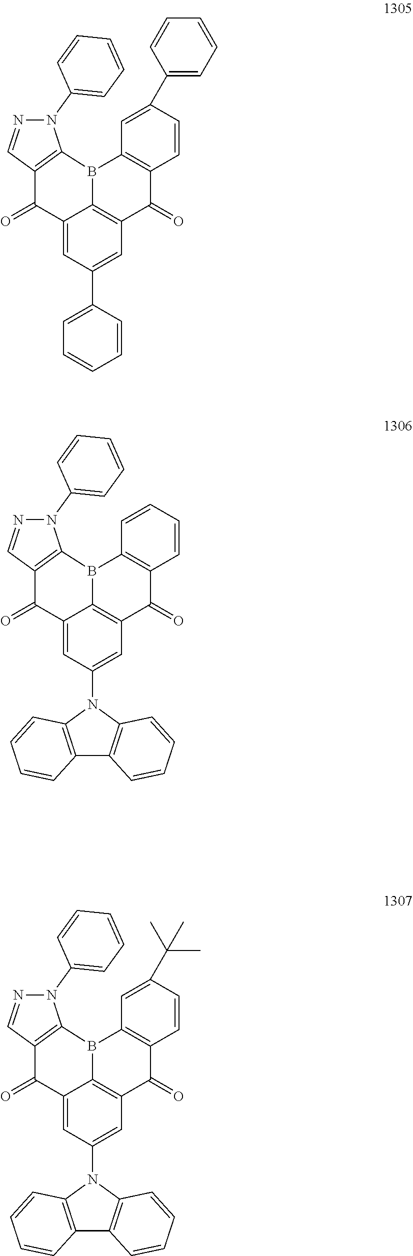 [SMILES: O=C1c2ccc(-c3ccccc3)cc2B2c3c1cc(-c1ccccc1)cc3C(=O)c1cnn(-c3ccccc3)c12 ; O=C1c2ccccc2B2c3c1cc(-n1c4ccccc4c4ccccc41)cc3C(=O)c1cnn(-c3ccccc3)c12 ; CC(C)(C)c1ccc2c(c1)B1c3c(cc(-n4c5ccccc5c5ccccc54)cc3C(=O)c3cnn(-c4ccccc4)c31)C2=O]

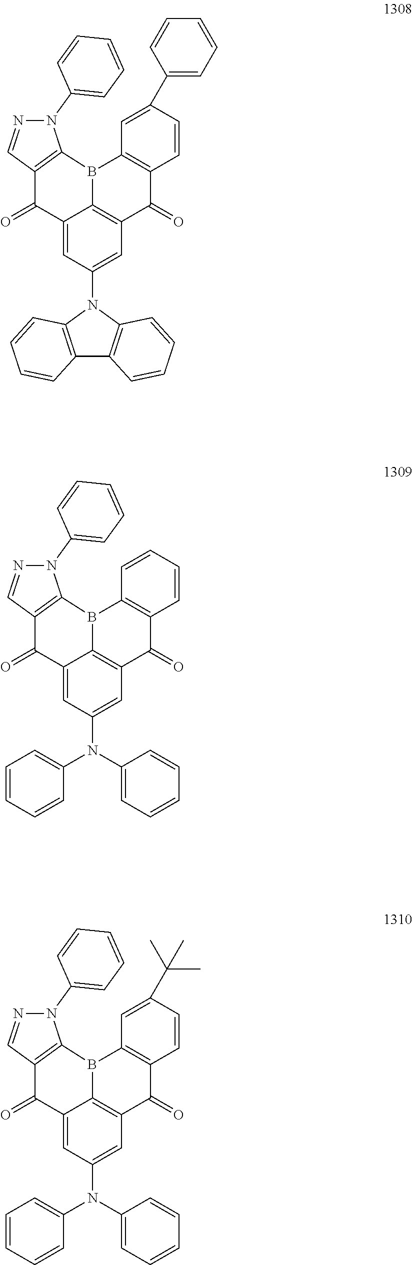 [SMILES: O=C1c2ccc(-c3ccccc3)cc2B2c3c1cc(-n1c4ccccc4c4ccccc41)cc3C(=O)c1cnn(-c3ccccc3)c12 ; O=C1c2ccccc2B2c3c1cc(N(c1ccccc1)c1ccccc1)cc3C(=O)c1cnn(-c3ccccc3)c12 ; CC(C)(C)c1ccc2c(c1)B1c3c(cc(N(c4ccccc4)c4ccccc4)cc3C(=O)c3cnn(-c4ccccc4)c31)C2=O]

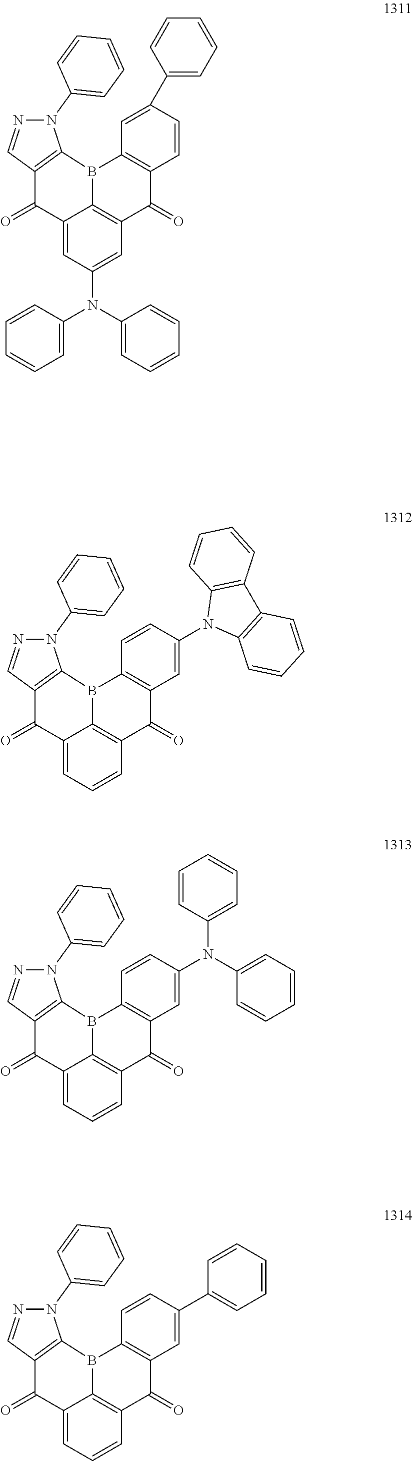 [SMILES: O=C1c2ccc(-c3ccccc3)cc2B2c3c1cc(N(c1ccccc1)c1ccccc1)cc3C(=O)c1cnn(-c3ccccc3)c12 ; O=C1c2cc(-n3c4ccccc4c4ccccc43)ccc2B2c3c1cccc3C(=O)c1cnn(-c3ccccc3)c12 ; O=C1c2cc(N(c3ccccc3)c3ccccc3)ccc2B2c3c1cccc3C(=O)c1cnn(-c3ccccc3)c12 ; O=C1c2cc(-c3ccccc3)ccc2B2c3c1cccc3C(=O)c1cnn(-c3ccccc3)c12]

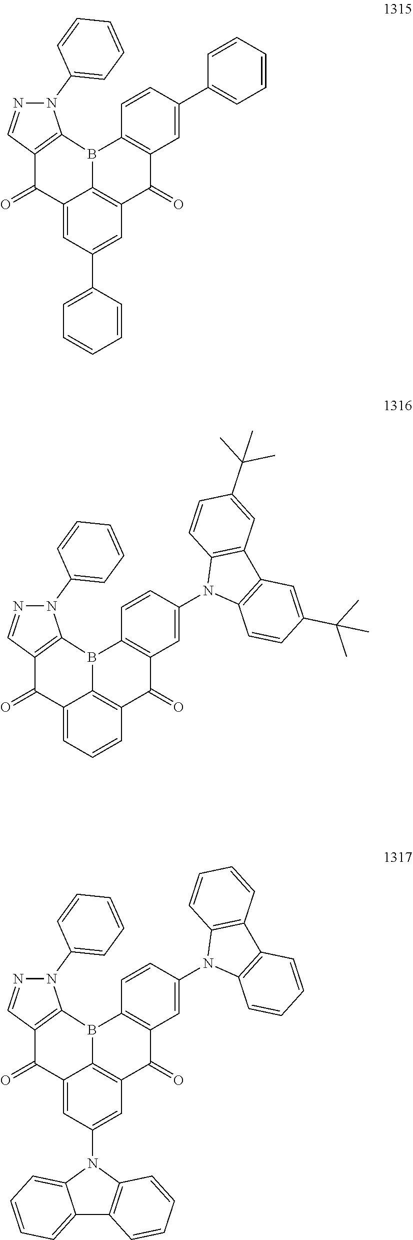 [SMILES: O=C1c2cc(-c3ccccc3)ccc2B2c3c1cc(-c1ccccc1)cc3C(=O)c1cnn(-c3ccccc3)c12 ; CC(C)(C)c1ccc2c(c1)c1cc(C(C)(C)C)ccc1n2-c1ccc2c(c1)C(=O)c1cccc3c1B2c1c(cnn1-c1ccccc1)C3=O ; O=C1c2cc(-n3c4ccccc4c4ccccc43)ccc2B2c3c1cc(-n1c4ccccc4c4ccccc41)cc3C(=O)c1cnn(-c3ccccc3)c12]

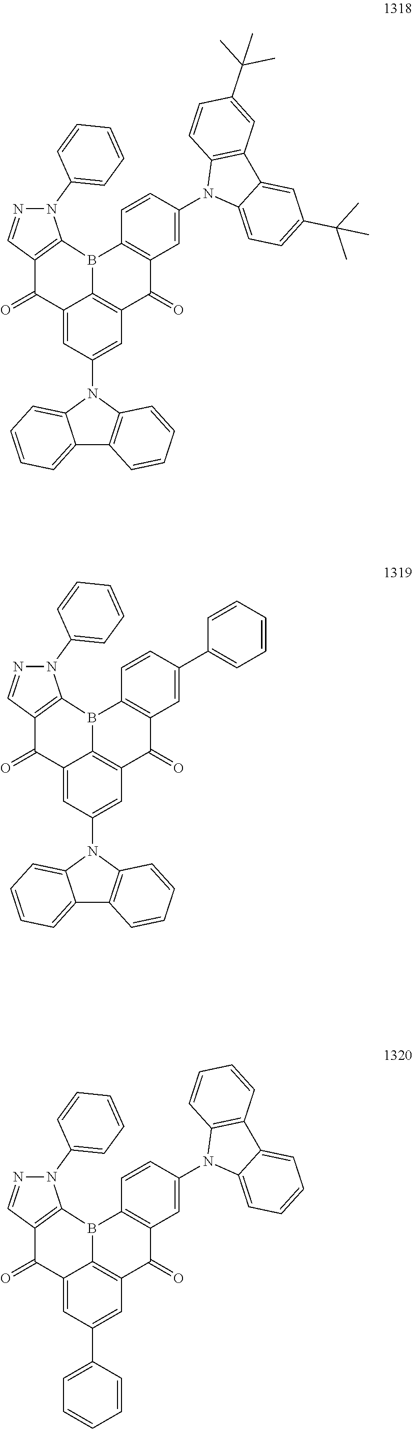 [SMILES: CC(C)(C)c1ccc2c(c1)c1cc(C(C)(C)C)ccc1n2-c1ccc2c(c1)C(=O)c1cc(-n3c4ccccc4c4ccccc43)cc3c1B2c1c(cnn1-c1ccccc1)C3=O ; O=C1c2cc(-c3ccccc3)ccc2B2c3c1cc(-n1c4ccccc4c4ccccc41)cc3C(=O)c1cnn(-c3ccccc3)c12 ; O=C1c2cc(-n3c4ccccc4c4ccccc43)ccc2B2c3c1cc(-c1ccccc1)cc3C(=O)c1cnn(-c3ccccc3)c12]

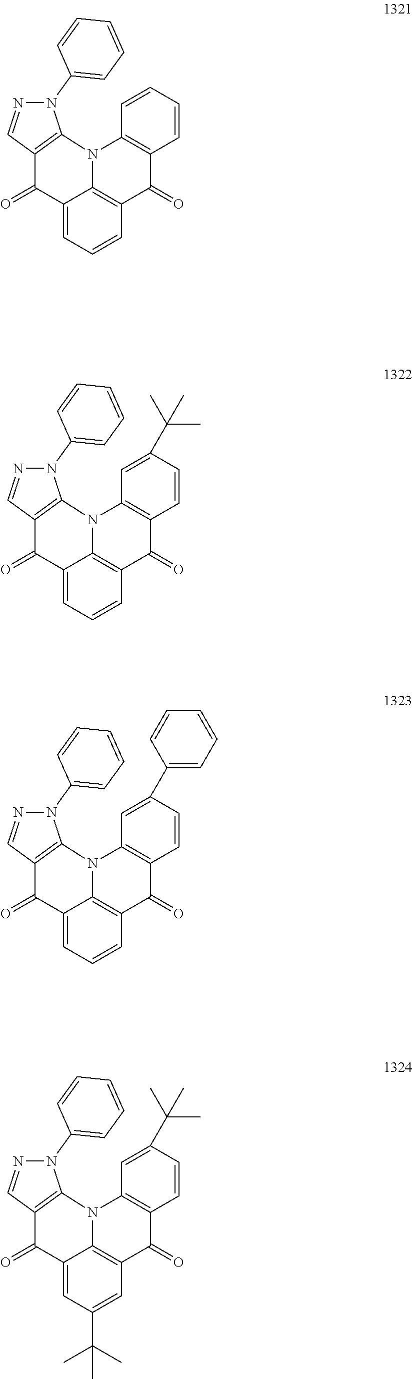 [SMILES: O=c1c2ccccc2n2c3c1cccc3c(=O)c1cnn(-c3ccccc3)c12 ; CC(C)(C)c1ccc2c(=O)c3cccc4c(=O)c5cnn(-c6ccccc6)c5n(c2c1)c34 ; O=c1c2ccc(-c3ccccc3)cc2n2c3c1cccc3c(=O)c1cnn(-c3ccccc3)c12 ; CC(C)(C)c1cc2c(=O)c3ccc(C(C)(C)C)cc3n3c2c(c1)c(=O)c1cnn(-c2ccccc2)c13]

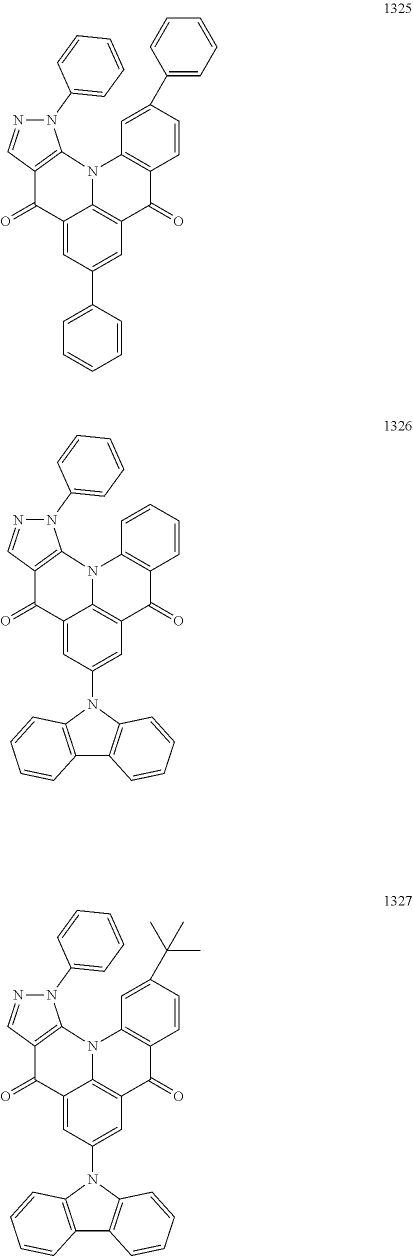 [SMILES: O=c1c2ccc(-c3ccccc3)cc2n2c3c1cc(-c1ccccc1)cc3c(=O)c1cnn(-c3ccccc3)c12 ; O=c1c2ccccc2n2c3c1cc(-n1c4ccccc4c4ccccc41)cc3c(=O)c1cnn(-c3ccccc3)c12 ; CC(C)(C)c1ccc2c(=O)c3cc(-n4c5ccccc5c5ccccc54)cc4c(=O)c5cnn(-c6ccccc6)c5n(c2c1)c34]

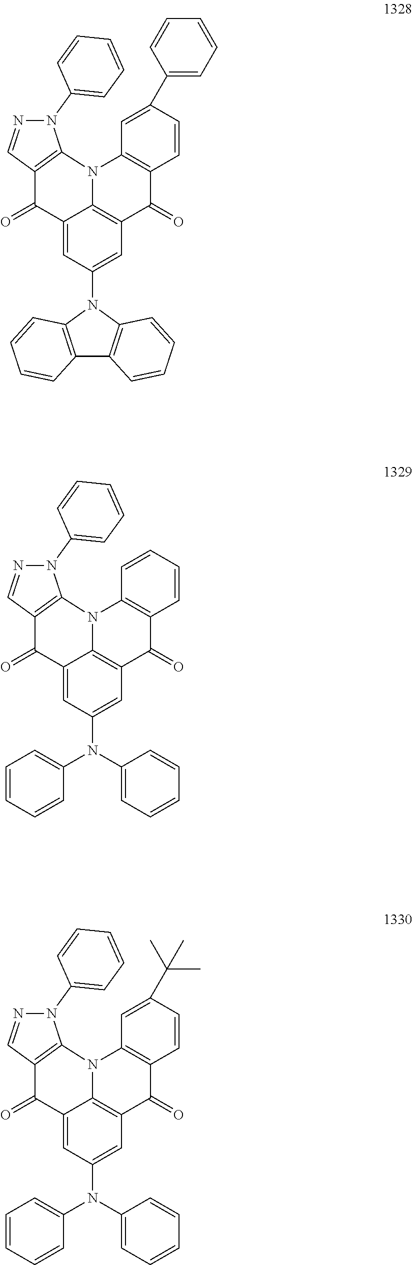 [SMILES: O=c1c2ccc(-c3ccccc3)cc2n2c3c1cc(-n1c4ccccc4c4ccccc41)cc3c(=O)c1cnn(-c3ccccc3)c12 ; O=c1c2ccccc2n2c3c1cc(N(c1ccccc1)c1ccccc1)cc3c(=O)c1cnn(-c3ccccc3)c12 ; CC(C)(C)c1ccc2c(=O)c3cc(N(c4ccccc4)c4ccccc4)cc4c(=O)c5cnn(-c6ccccc6)c5n(c2c1)c34]

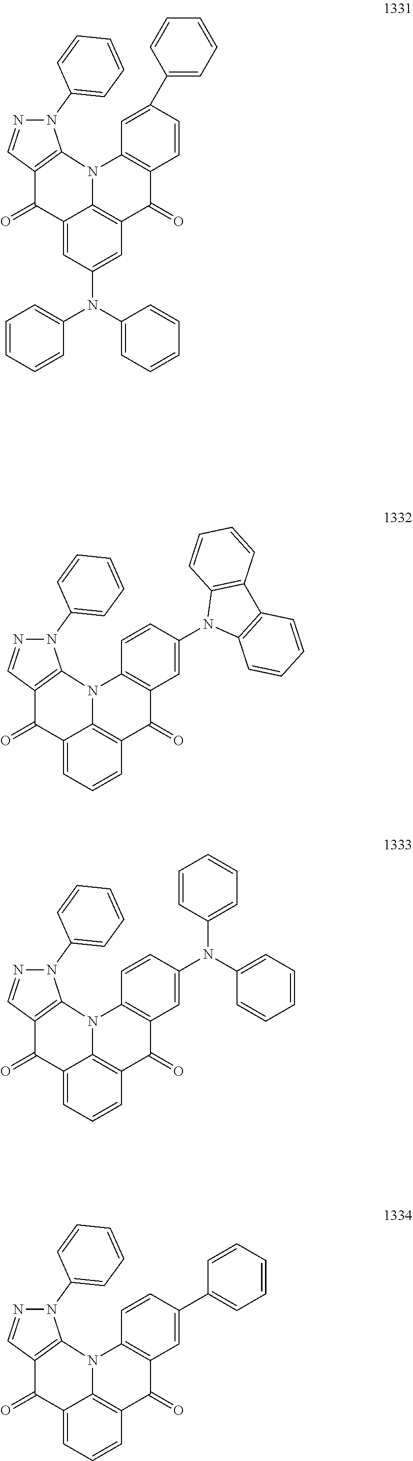 [SMILES: O=c1c2ccc(-c3ccccc3)cc2n2c3c1cc(N(c1ccccc1)c1ccccc1)cc3c(=O)c1cnn(-c3ccccc3)c12 ; O=c1c2cc(-n3c4ccccc4c4ccccc43)ccc2n2c3c1cccc3c(=O)c1cnn(-c3ccccc3)c12 ; O=c1c2cc(N(c3ccccc3)c3ccccc3)ccc2n2c3c1cccc3c(=O)c1cnn(-c3ccccc3)c12 ; O=c1c2cc(-c3ccccc3)ccc2n2c3c1cccc3c(=O)c1cnn(-c3ccccc3)c12]

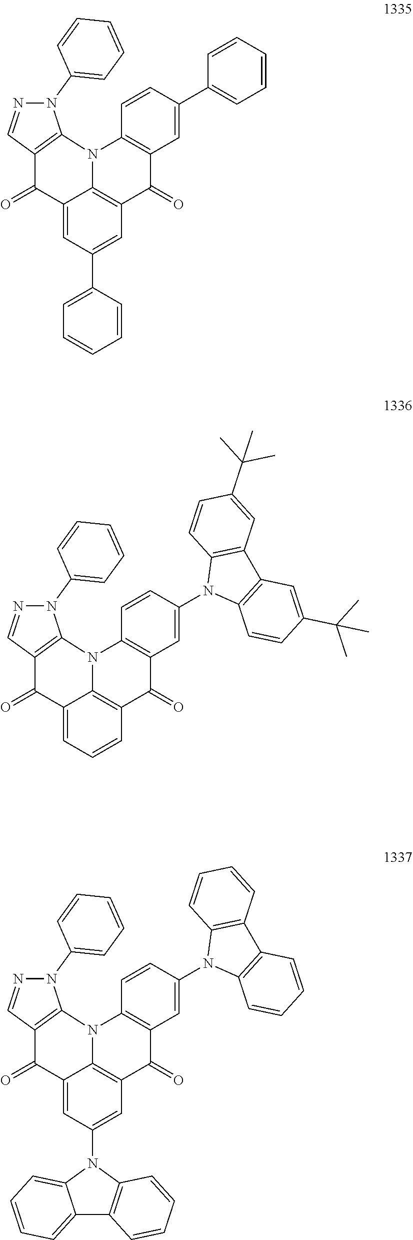 [SMILES: O=c1c2cc(-c3ccccc3)ccc2n2c3c1cc(-c1ccccc1)cc3c(=O)c1cnn(-c3ccccc3)c12 ; CC(C)(C)c1ccc2c(c1)c1cc(C(C)(C)C)ccc1n2-c1ccc2c(c1)c(=O)c1cccc3c(=O)c4cnn(-c5ccccc5)c4n2c13 ; O=c1c2cc(-n3c4ccccc4c4ccccc43)ccc2n2c3c1cc(-n1c4ccccc4c4ccccc41)cc3c(=O)c1cnn(-c3ccccc3)c12]

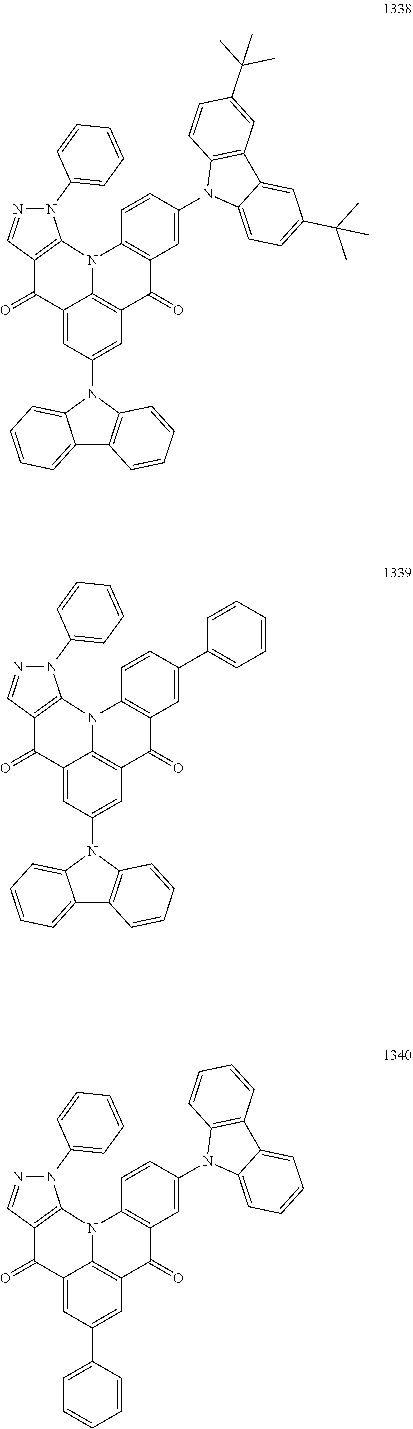 [SMILES: CC(C)(C)c1ccc2c(c1)c1cc(C(C)(C)C)ccc1n2-c1ccc2c(c1)c(=O)c1cc(-n3c4ccccc4c4ccccc43)cc3c(=O)c4cnn(-c5ccccc5)c4n2c13 ; O=c1c2cc(-c3ccccc3)ccc2n2c3c1cc(-n1c4ccccc4c4ccccc41)cc3c(=O)c1cnn(-c3ccccc3)c12 ; O=c1c2cc(-n3c4ccccc4c4ccccc43)ccc2n2c3c1cc(-c1ccccc1)cc3c(=O)c1cnn(-c3ccccc3)c12]

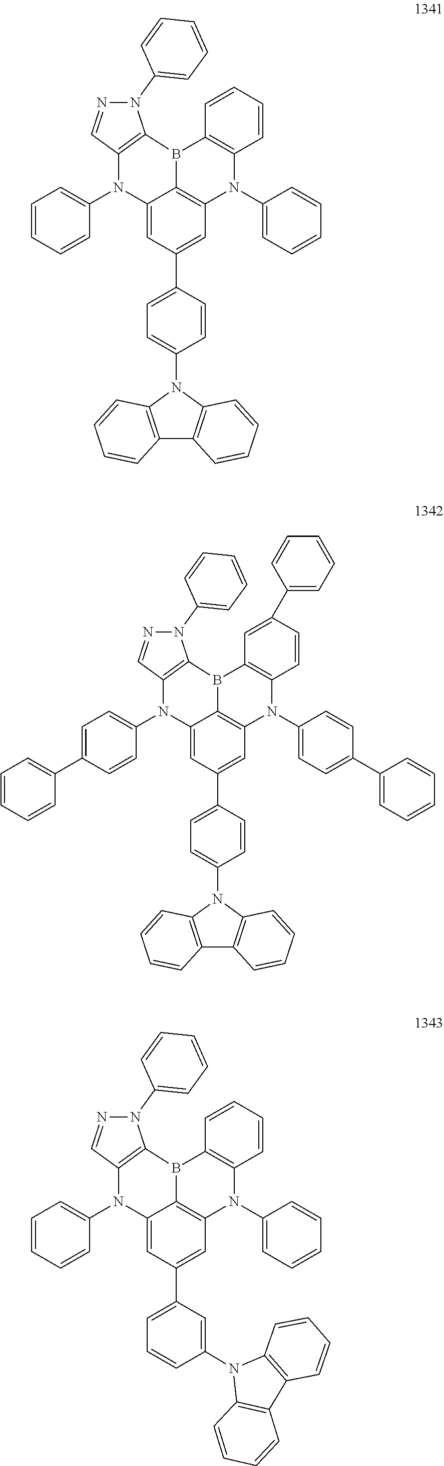 [SMILES: c1ccc(N2c3ccccc3B3c4c2cc(-c2ccc(-n5c6ccccc6c6ccccc65)cc2)cc4N(c2ccccc2)c2cnn(-c4ccccc4)c23)cc1 ; c1ccc(-c2ccc(N3c4ccc(-c5ccccc5)cc4B4c5c3cc(-c3ccc(-n6c7ccccc7c7ccccc76)cc3)cc5N(c3ccc(-c5ccccc5)cc3)c3cnn(-c5ccccc5)c34)cc2)cc1 ; c1ccc(N2c3ccccc3B3c4c2cc(-c2cccc(-n5c6ccccc6c6ccccc65)c2)cc4N(c2ccccc2)c2cnn(-c4ccccc4)c23)cc1]

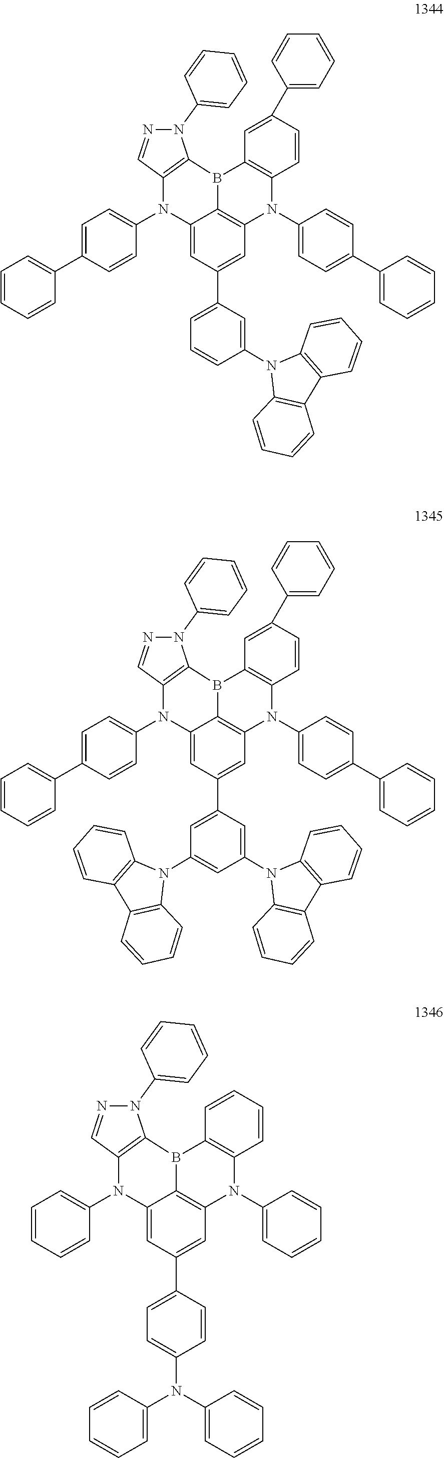 [SMILES: c1ccc(-c2ccc(N3c4ccc(-c5ccccc5)cc4B4c5c3cc(-c3cccc(-n6c7ccccc7c7ccccc76)c3)cc5N(c3ccc(-c5ccccc5)cc3)c3cnn(-c5ccccc5)c34)cc2)cc1 ; c1ccc(-c2ccc(N3c4ccc(-c5ccccc5)cc4B4c5c3cc(-c3cc(-n6c7ccccc7c7ccccc76)cc(-n6c7ccccc7c7ccccc76)c3)cc5N(c3ccc(-c5ccccc5)cc3)c3cnn(-c5ccccc5)c34)cc2)cc1 ; c1ccc(N(c2ccccc2)c2ccc(-c3cc4c5c(c3)N(c3ccccc3)c3cnn(-c6ccccc6)c3B5c3ccccc3N4c3ccccc3)cc2)cc1]

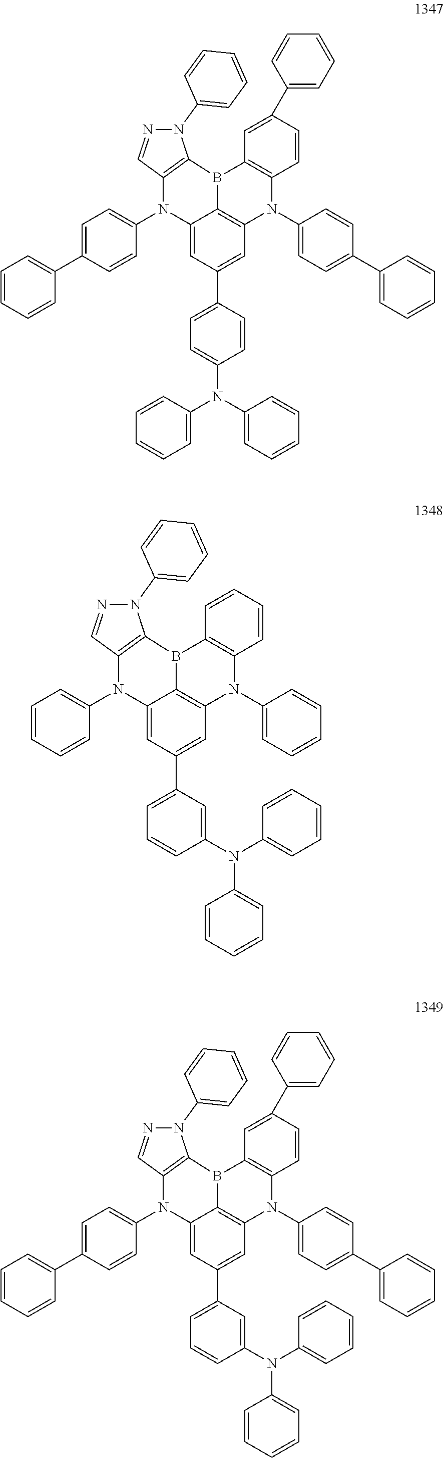 [SMILES: c1ccc(-c2ccc(N3c4ccc(-c5ccccc5)cc4B4c5c3cc(-c3ccc(N(c6ccccc6)c6ccccc6)cc3)cc5N(c3ccc(-c5ccccc5)cc3)c3cnn(-c5ccccc5)c34)cc2)cc1 ; c1ccc(N(c2ccccc2)c2cccc(-c3cc4c5c(c3)N(c3ccccc3)c3cnn(-c6ccccc6)c3B5c3ccccc3N4c3ccccc3)c2)cc1 ; c1ccc(-c2ccc(N3c4ccc(-c5ccccc5)cc4B4c5c3cc(-c3cccc(N(c6ccccc6)c6ccccc6)c3)cc5N(c3ccc(-c5ccccc5)cc3)c3cnn(-c5ccccc5)c34)cc2)cc1]

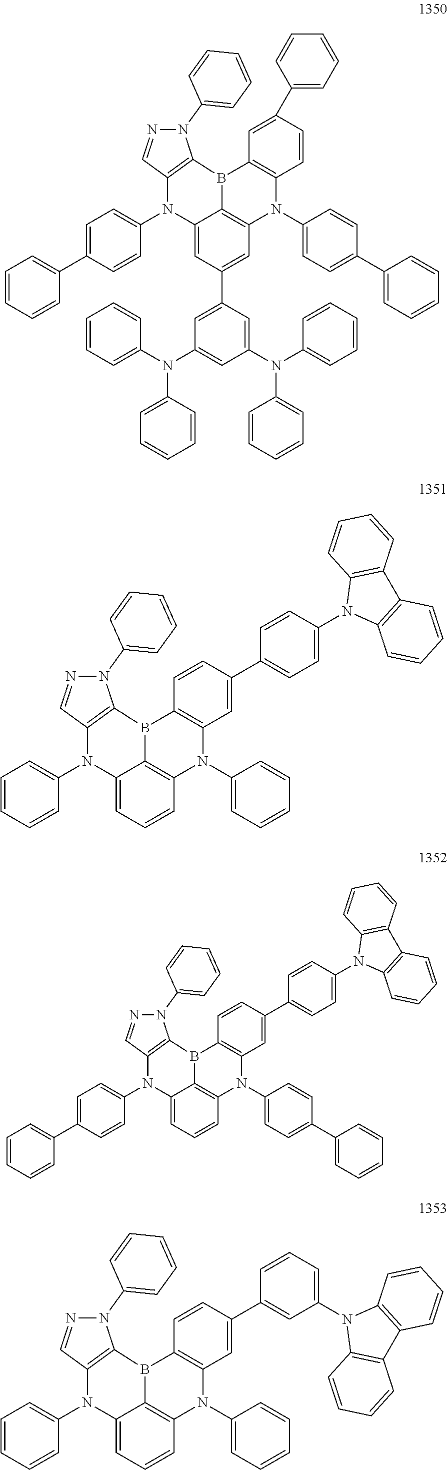 [SMILES: c1ccc(-c2ccc(N3c4ccc(-c5ccccc5)cc4B4c5c3cc(-c3cc(N(c6ccccc6)c6ccccc6)cc(N(c6ccccc6)c6ccccc6)c3)cc5N(c3ccc(-c5ccccc5)cc3)c3cnn(-c5ccccc5)c34)cc2)cc1 ; c1ccc(N2c3cc(-c4ccc(-n5c6ccccc6c6ccccc65)cc4)ccc3B3c4c2cccc4N(c2ccccc2)c2cnn(-c4ccccc4)c23)cc1 ; c1ccc(-c2ccc(N3c4cc(-c5ccc(-n6c7ccccc7c7ccccc76)cc5)ccc4B4c5c3cccc5N(c3ccc(-c5ccccc5)cc3)c3cnn(-c5ccccc5)c34)cc2)cc1 ; c1ccc(N2c3cc(-c4cccc(-n5c6ccccc6c6ccccc65)c4)ccc3B3c4c2cccc4N(c2ccccc2)c2cnn(-c4ccccc4)c23)cc1]

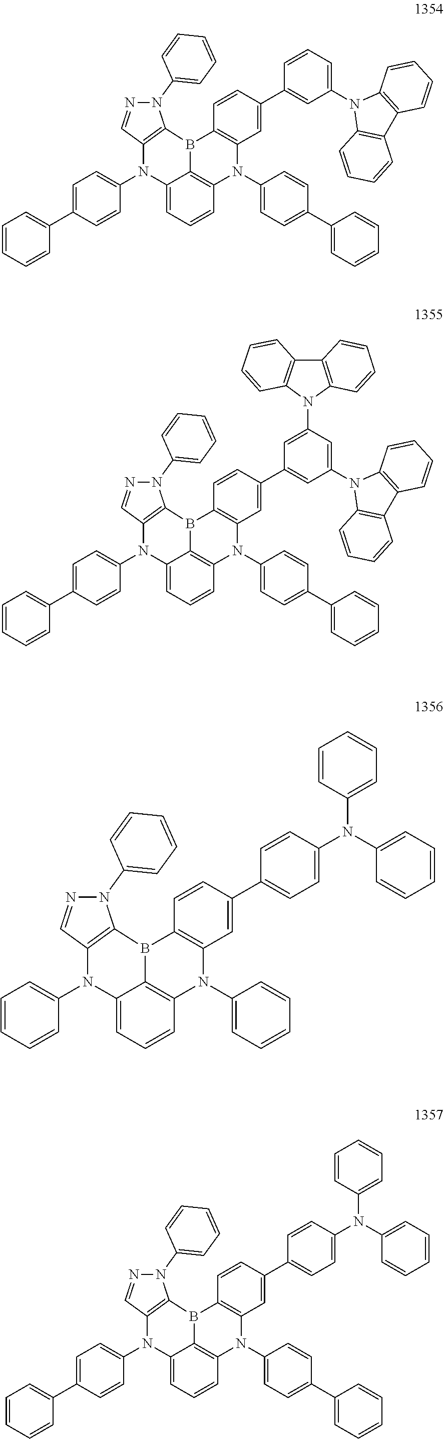 [SMILES: c1ccc(-c2ccc(N3c4cc(-c5cccc(-n6c7ccccc7c7ccccc76)c5)ccc4B4c5c3cccc5N(c3ccc(-c5ccccc5)cc3)c3cnn(-c5ccccc5)c34)cc2)cc1 ; c1ccc(-c2ccc(N3c4cc(-c5cc(-n6c7ccccc7c7ccccc76)cc(-n6c7ccccc7c7ccccc76)c5)ccc4B4c5c3cccc5N(c3ccc(-c5ccccc5)cc3)c3cnn(-c5ccccc5)c34)cc2)cc1 ; c1ccc(N(c2ccccc2)c2ccc(-c3ccc4c(c3)N(c3ccccc3)c3cccc5c3B4c3c(cnn3-c3ccccc3)N5c3ccccc3)cc2)cc1 ; c1ccc(-c2ccc(N3c4cc(-c5ccc(N(c6ccccc6)c6ccccc6)cc5)ccc4B4c5c3cccc5N(c3ccc(-c5ccccc5)cc3)c3cnn(-c5ccccc5)c34)cc2)cc1]

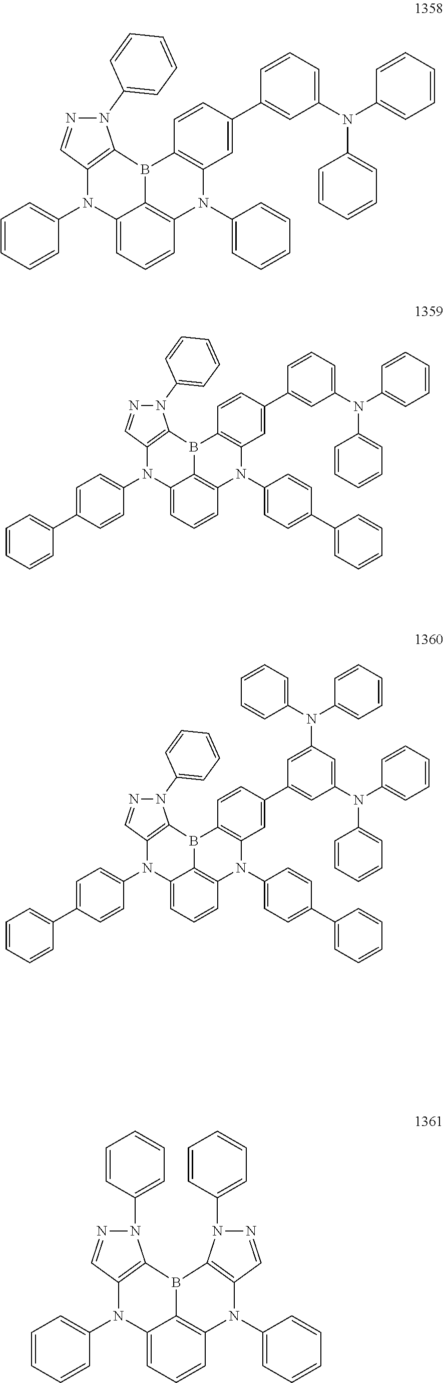 [SMILES: c1ccc(N(c2ccccc2)c2cccc(-c3ccc4c(c3)N(c3ccccc3)c3cccc5c3B4c3c(cnn3-c3ccccc3)N5c3ccccc3)c2)cc1 ; c1ccc(-c2ccc(N3c4cc(-c5cccc(N(c6ccccc6)c6ccccc6)c5)ccc4B4c5c3cccc5N(c3ccc(-c5ccccc5)cc3)c3cnn(-c5ccccc5)c34)cc2)cc1 ; c1ccc(-c2ccc(N3c4cc(-c5cc(N(c6ccccc6)c6ccccc6)cc(N(c6ccccc6)c6ccccc6)c5)ccc4B4c5c3cccc5N(c3ccc(-c5ccccc5)cc3)c3cnn(-c5ccccc5)c34)cc2)cc1 ; c1ccc(N2c3cccc4c3B(c3c2cnn3-c2ccccc2)c2c(cnn2-c2ccccc2)N4c2ccccc2)cc1]

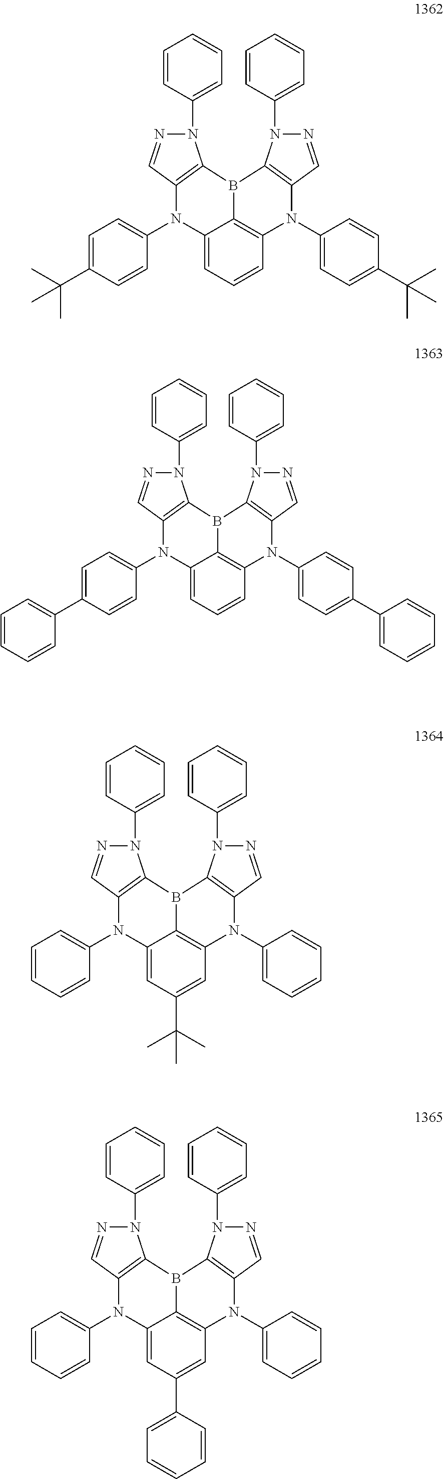 [SMILES: CC(C)(C)c1ccc(N2c3cccc4c3B(c3c2cnn3-c2ccccc2)c2c(cnn2-c2ccccc2)N4c2ccc(C(C)(C)C)cc2)cc1 ; c1ccc(-c2ccc(N3c4cccc5c4B(c4c3cnn4-c3ccccc3)c3c(cnn3-c3ccccc3)N5c3ccc(-c4ccccc4)cc3)cc2)cc1 ; CC(C)(C)c1cc2c3c(c1)N(c1ccccc1)c1cnn(-c4ccccc4)c1B3c1c(cnn1-c1ccccc1)N2c1ccccc1 ; c1ccc(-c2cc3c4c(c2)N(c2ccccc2)c2cnn(-c5ccccc5)c2B4c2c(cnn2-c2ccccc2)N3c2ccccc2)cc1]

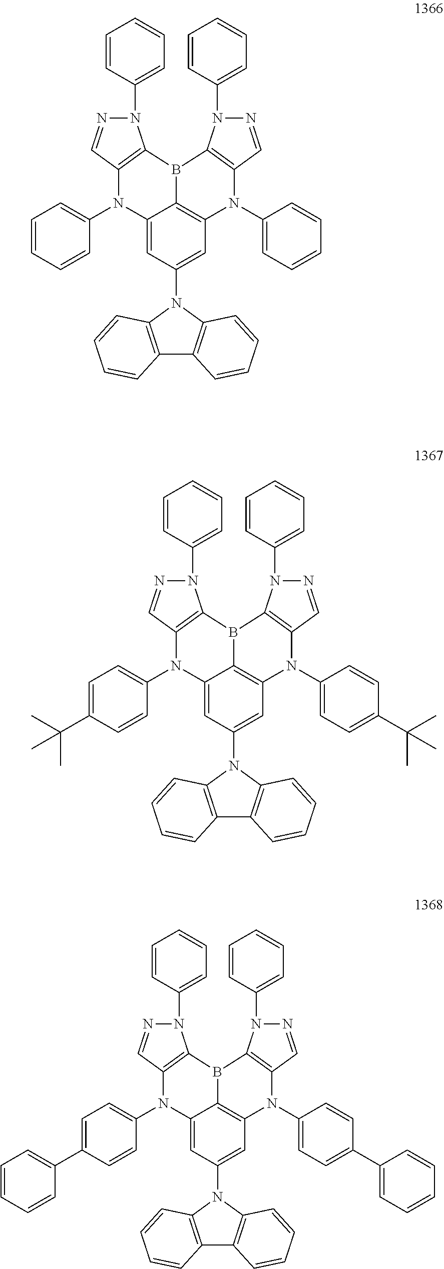 [SMILES: c1ccc(N2c3cc(-n4c5ccccc5c5ccccc54)cc4c3B(c3c2cnn3-c2ccccc2)c2c(cnn2-c2ccccc2)N4c2ccccc2)cc1 ; CC(C)(C)c1ccc(N2c3cc(-n4c5ccccc5c5ccccc54)cc4c3B(c3c2cnn3-c2ccccc2)c2c(cnn2-c2ccccc2)N4c2ccc(C(C)(C)C)cc2)cc1 ; c1ccc(-c2ccc(N3c4cc(-n5c6ccccc6c6ccccc65)cc5c4B(c4c3cnn4-c3ccccc3)c3c(cnn3-c3ccccc3)N5c3ccc(-c4ccccc4)cc3)cc2)cc1]

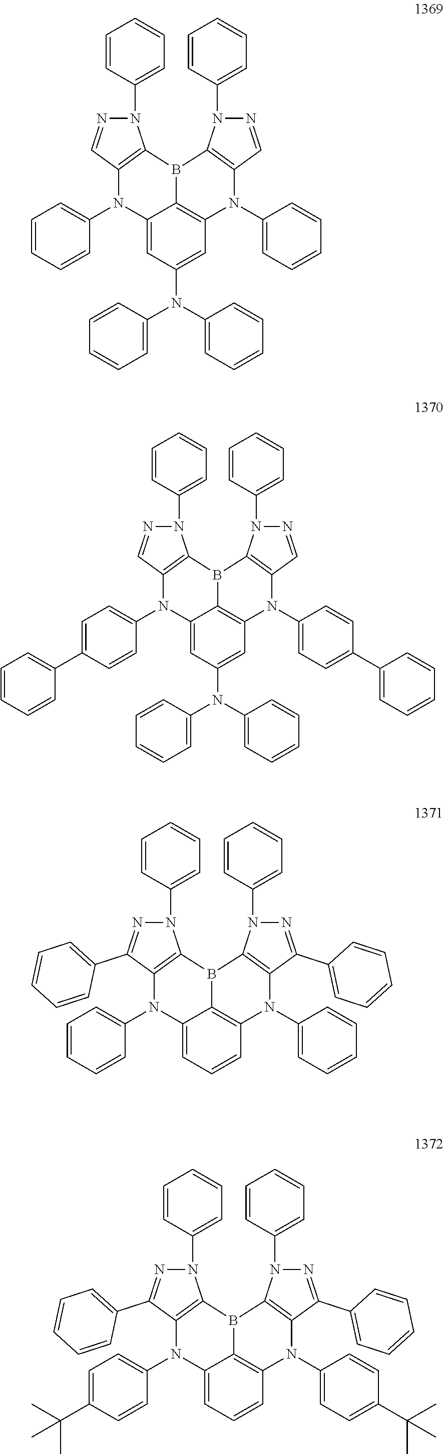 [SMILES: c1ccc(N(c2ccccc2)c2cc3c4c(c2)N(c2ccccc2)c2cnn(-c5ccccc5)c2B4c2c(cnn2-c2ccccc2)N3c2ccccc2)cc1 ; c1ccc(-c2ccc(N3c4cc(N(c5ccccc5)c5ccccc5)cc5c4B(c4c3cnn4-c3ccccc3)c3c(cnn3-c3ccccc3)N5c3ccc(-c4ccccc4)cc3)cc2)cc1 ; c1ccc(-c2nn(-c3ccccc3)c3c2N(c2ccccc2)c2cccc4c2B3c2c(c(-c3ccccc3)nn2-c2ccccc2)N4c2ccccc2)cc1 ; CC(C)(C)c1ccc(N2c3cccc4c3B(c3c2c(-c2ccccc2)nn3-c2ccccc2)c2c(c(-c3ccccc3)nn2-c2ccccc2)N4c2ccc(C(C)(C)C)cc2)cc1]

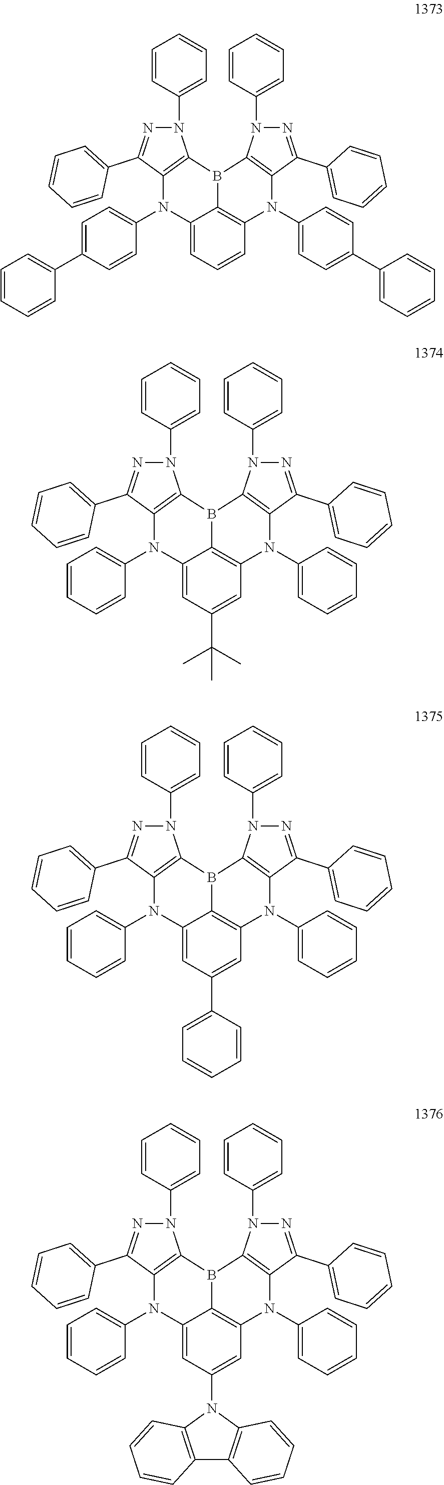 [SMILES: c1ccc(-c2ccc(N3c4cccc5c4B(c4c3c(-c3ccccc3)nn4-c3ccccc3)c3c(c(-c4ccccc4)nn3-c3ccccc3)N5c3ccc(-c4ccccc4)cc3)cc2)cc1 ; CC(C)(C)c1cc2c3c(c1)N(c1ccccc1)c1c(-c4ccccc4)nn(-c4ccccc4)c1B3c1c(c(-c3ccccc3)nn1-c1ccccc1)N2c1ccccc1 ; c1ccc(-c2cc3c4c(c2)N(c2ccccc2)c2c(-c5ccccc5)nn(-c5ccccc5)c2B4c2c(c(-c4ccccc4)nn2-c2ccccc2)N3c2ccccc2)cc1 ; c1ccc(-c2nn(-c3ccccc3)c3c2N(c2ccccc2)c2cc(-n4c5ccccc5c5ccccc54)cc4c2B3c2c(c(-c3ccccc3)nn2-c2ccccc2)N4c2ccccc2)cc1]

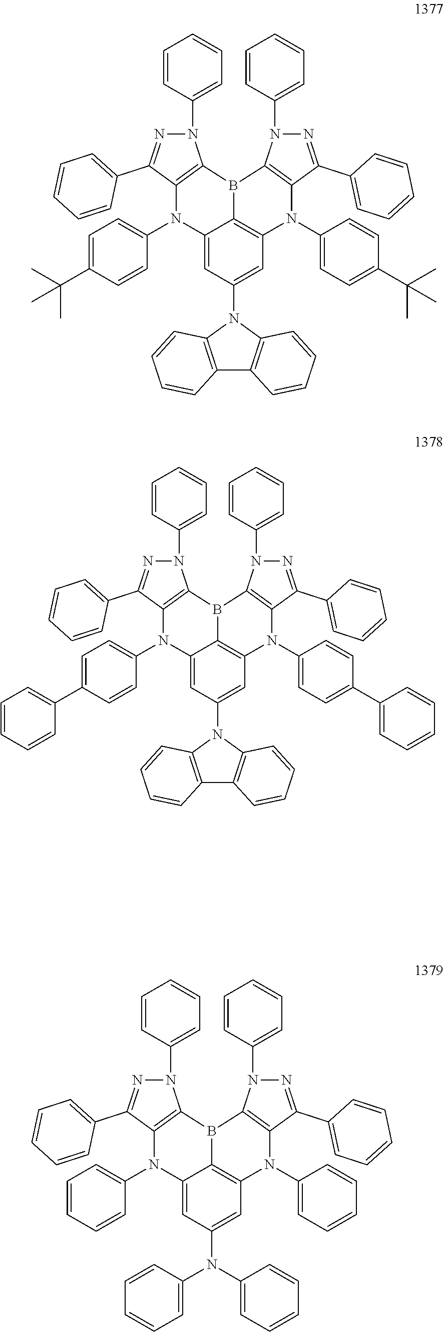 [SMILES: CC(C)(C)c1ccc(N2c3cc(-n4c5ccccc5c5ccccc54)cc4c3B(c3c2c(-c2ccccc2)nn3-c2ccccc2)c2c(c(-c3ccccc3)nn2-c2ccccc2)N4c2ccc(C(C)(C)C)cc2)cc1 ; c1ccc(-c2ccc(N3c4cc(-n5c6ccccc6c6ccccc65)cc5c4B(c4c3c(-c3ccccc3)nn4-c3ccccc3)c3c(c(-c4ccccc4)nn3-c3ccccc3)N5c3ccc(-c4ccccc4)cc3)cc2)cc1 ; c1ccc(-c2nn(-c3ccccc3)c3c2N(c2ccccc2)c2cc(N(c4ccccc4)c4ccccc4)cc4c2B3c2c(c(-c3ccccc3)nn2-c2ccccc2)N4c2ccccc2)cc1]

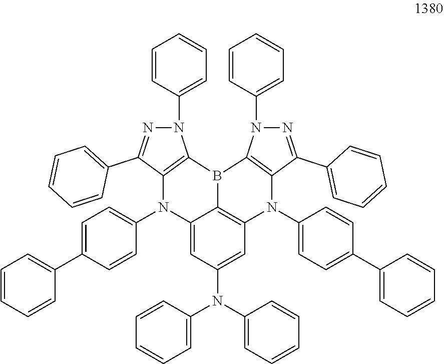 [SMILES: c1ccc(-c2ccc(N3c4cc(N(c5ccccc5)c5ccccc5)cc5c4B(c4c3c(-c3ccccc3)nn4-c3ccccc3)c3c(c(-c4ccccc4)nn3-c3ccccc3)N5c3ccc(-c4ccccc4)cc3)cc2)cc1]

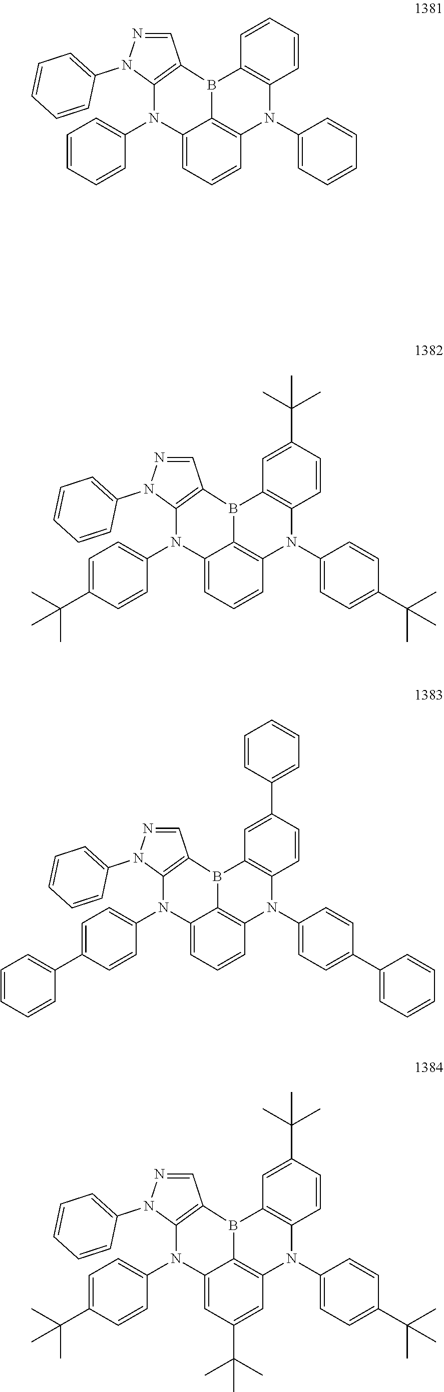

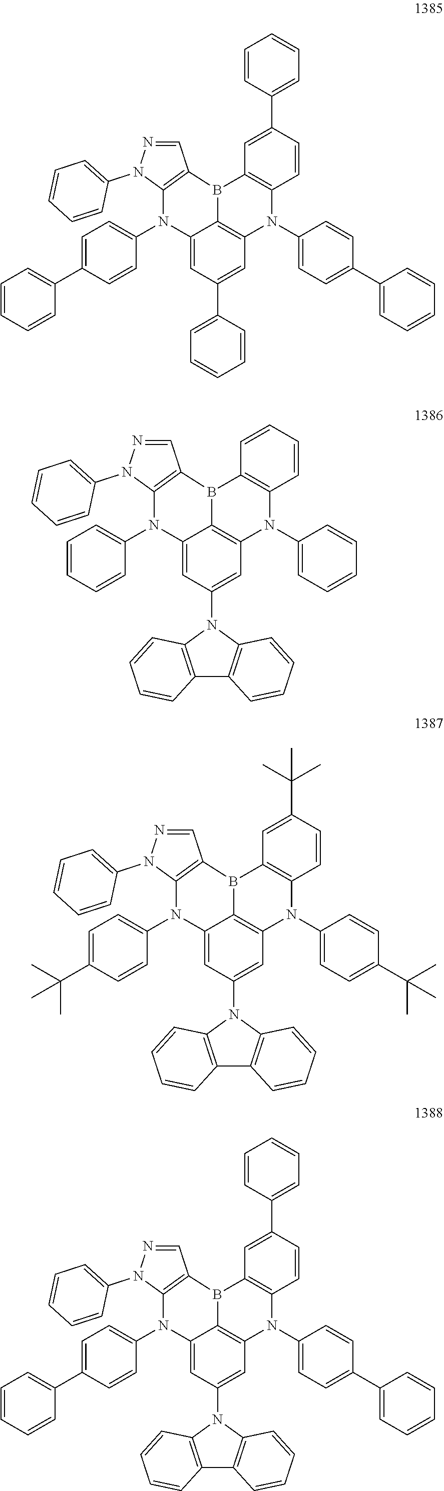 [SMILES: c1ccc(-c2ccc(N3c4ccc(-c5ccccc5)cc4B4c5cnn(-c6ccccc6)c5N(c5ccc(-c6ccccc6)cc5)c5cc(-c6ccccc6)cc3c54)cc2)cc1 ; c1ccc(N2c3ccccc3B3c4cnn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3B3c4cnn(-c5ccccc5)c4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4B4c5cnn(-c6ccccc6)c5N(c5ccc(-c6ccccc6)cc5)c5cc(-n6c7ccccc7c7ccccc76)cc3c54)cc2)cc1]

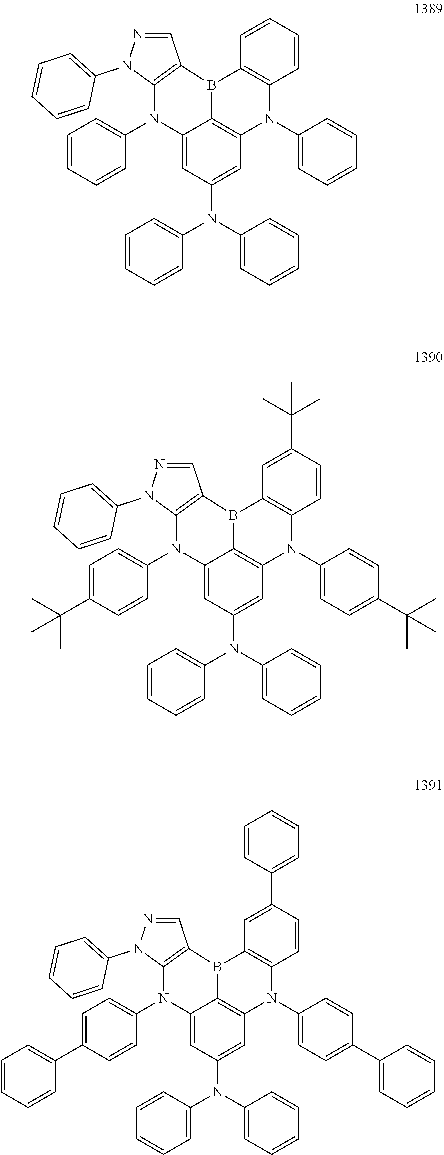 [SMILES: c1ccc(N(c2ccccc2)c2cc3c4c(c2)N(c2ccccc2)c2c(cnn2-c2ccccc2)B4c2ccccc2N3c2ccccc2)cc1 ; CC(C)(C)c1ccc(N2c3ccc(C(C)(C)C)cc3B3c4cnn(-c5ccccc5)c4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5cnn(-c6ccccc6)c5N(c5ccc(-c6ccccc6)cc5)c5cc(N(c6ccccc6)c6ccccc6)cc3c54)cc2)cc1]

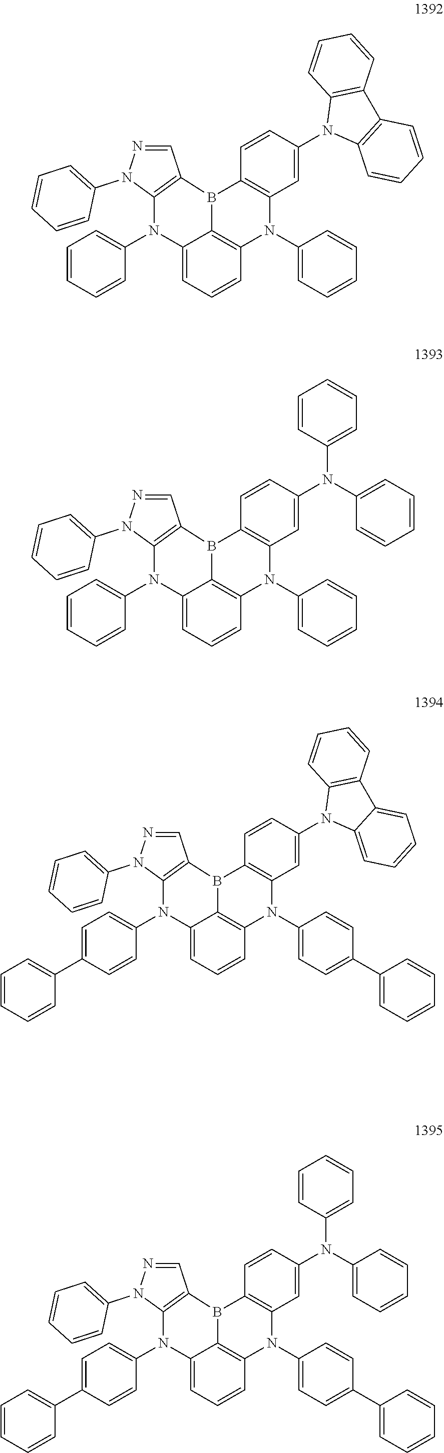 [SMILES: c1ccc(N2c3cc(-n4c5ccccc5c5ccccc54)ccc3B3c4cnn(-c5ccccc5)c4N(c4ccccc4)c4cccc2c43)cc1 ; c1ccc(N(c2ccccc2)c2ccc3c(c2)N(c2ccccc2)c2cccc4c2B3c2cnn(-c3ccccc3)c2N4c2ccccc2)cc1 ; c1ccc(-c2ccc(N3c4cc(-n5c6ccccc6c6ccccc65)ccc4B4c5cnn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5cnn(-c6ccccc6)c5N(c5ccc(-c6ccccc6)cc5)c5cccc3c54)cc2)cc1]

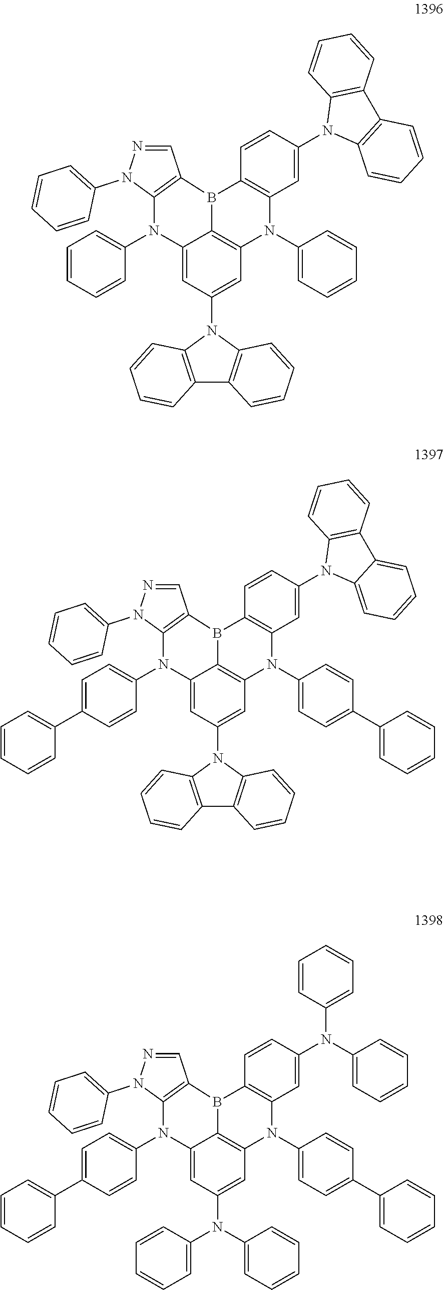 [SMILES: c1ccc(N2c3cc(-n4c5ccccc5c5ccccc54)ccc3B3c4cnn(-c5ccccc5)c4N(c4ccccc4)c4cc(-n5c6ccccc6c6ccccc65)cc2c43)cc1 ; c1ccc(-c2ccc(N3c4cc(-n5c6ccccc6c6ccccc65)ccc4B4c5cnn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cc(N(c5ccccc5)c5ccccc5)ccc4B4c5cnn(-c6ccccc6)c5N(c5ccc(-c6ccccc6)cc5)c5cc(N(c6ccccc6)c6ccccc6)cc3c54)cc2)cc1]

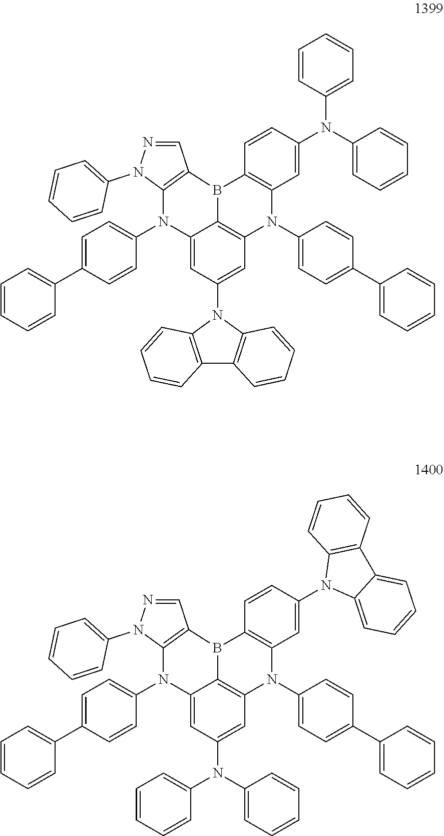 [SMILES: c1ccc(-c2ccc(N3c4cc(N(c5ccccc5)c5ccccc5)ccc4B4c5cnn(-c6ccccc6)c5N(c5ccc(-c6ccccc6)cc5)c5cc(-n6c7ccccc7c7ccccc76)cc3c54)cc2)cc1 ; c1ccc(-c2ccc(N3c4cc(-n5c6ccccc6c6ccccc65)ccc4B4c5cnn(-c6ccccc6)c5N(c5ccc(-c6ccccc6)cc5)c5cc(N(c6ccccc6)c6ccccc6)cc3c54)cc2)cc1]

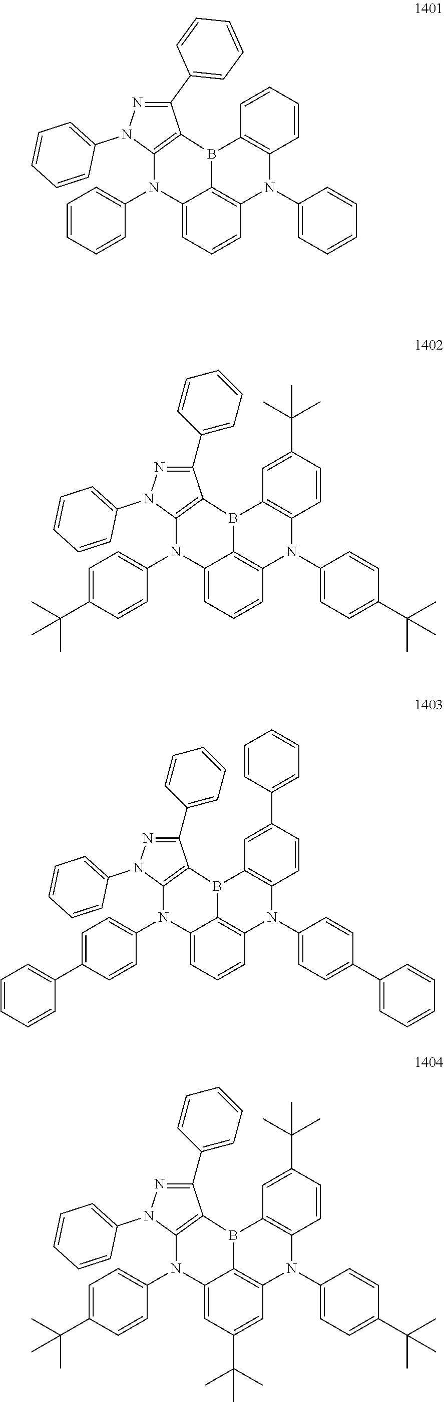

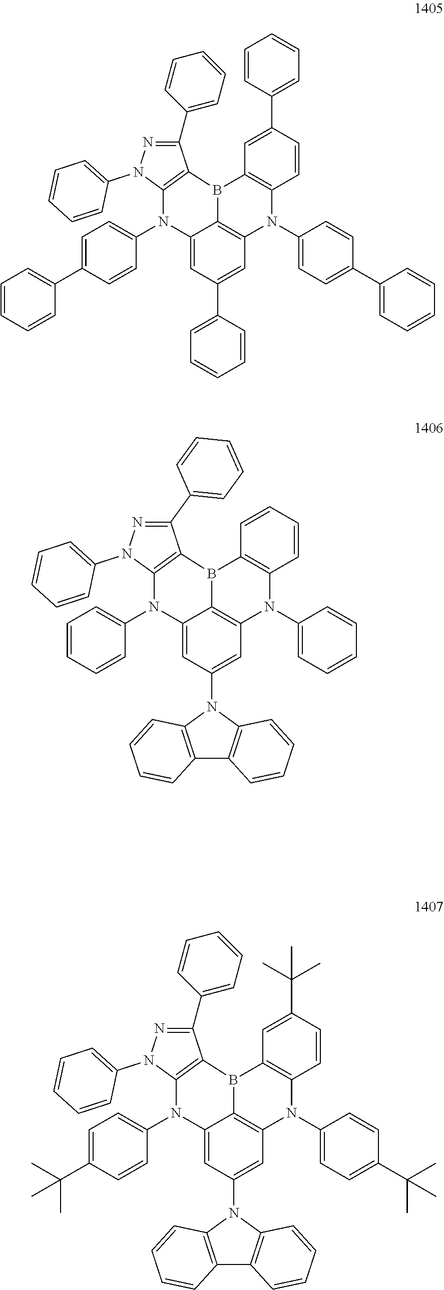 [SMILES: c1ccc(-c2ccc(N3c4ccc(-c5ccccc5)cc4B4c5c3cc(-c3ccccc3)cc5N(c3ccc(-c5ccccc5)cc3)c3c4c(-c4ccccc4)nn3-c3ccccc3)cc2)cc1 ; c1ccc(-c2nn(-c3ccccc3)c3c2B2c4ccccc4N(c4ccccc4)c4cc(-n5c6ccccc6c6ccccc65)cc(c42)N3c2ccccc2)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2c3c(-c3ccccc3)nn2-c2ccccc2)cc1]

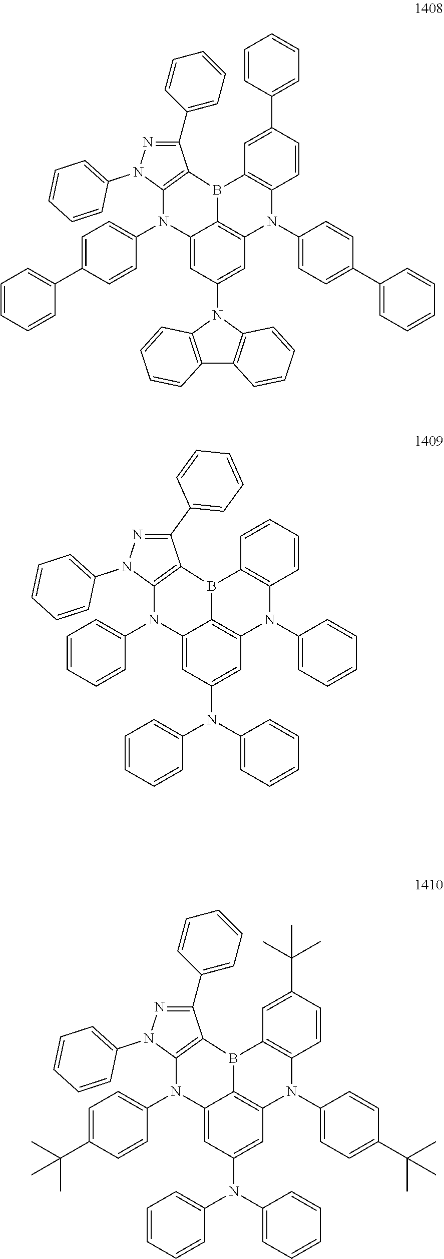 [SMILES: c1ccc(-c2ccc(N3c4ccc(-c5ccccc5)cc4B4c5c3cc(-n3c6ccccc6c6ccccc63)cc5N(c3ccc(-c5ccccc5)cc3)c3c4c(-c4ccccc4)nn3-c3ccccc3)cc2)cc1 ; c1ccc(-c2nn(-c3ccccc3)c3c2B2c4ccccc4N(c4ccccc4)c4cc(N(c5ccccc5)c5ccccc5)cc(c42)N3c2ccccc2)cc1 ; CC(C)(C)c1ccc(N2c3ccc(C(C)(C)C)cc3B3c4c2cc(N(c2ccccc2)c2ccccc2)cc4N(c2ccc(C(C)(C)C)cc2)c2c3c(-c3ccccc3)nn2-c2ccccc2)cc1]

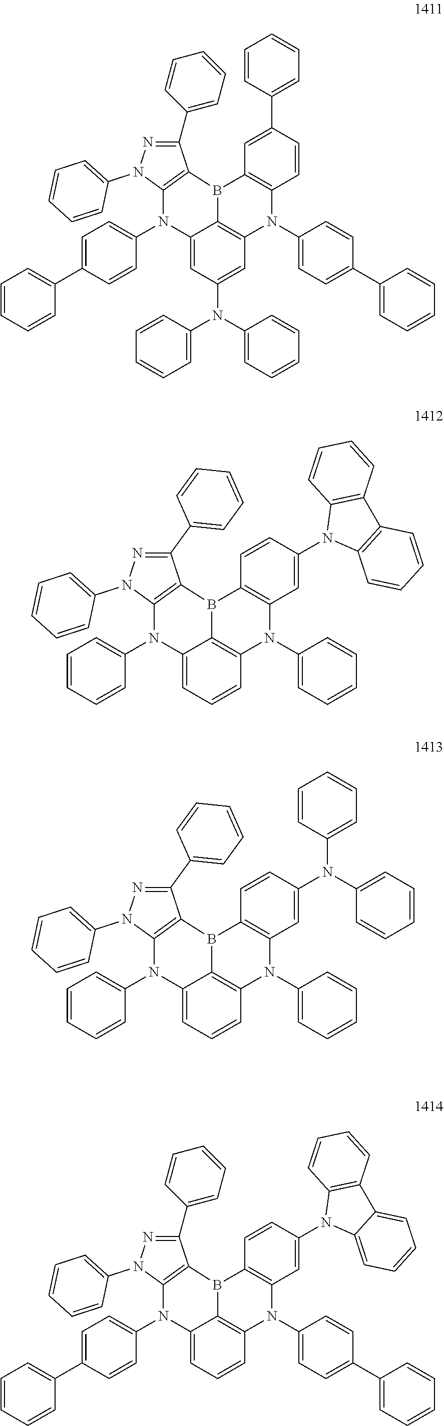 [SMILES: c1ccc(-c2ccc(N3c4ccc(-c5ccccc5)cc4B4c5c3cc(N(c3ccccc3)c3ccccc3)cc5N(c3ccc(-c5ccccc5)cc3)c3c4c(-c4ccccc4)nn3-c3ccccc3)cc2)cc1 ; c1ccc(-c2nn(-c3ccccc3)c3c2B2c4ccc(-n5c6ccccc6c6ccccc65)cc4N(c4ccccc4)c4cccc(c42)N3c2ccccc2)cc1 ; c1ccc(-c2nn(-c3ccccc3)c3c2B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5c3cccc5N(c3ccc(-c5ccccc5)cc3)c3c4c(-c4ccccc4)nn3-c3ccccc3)cc2)cc1]

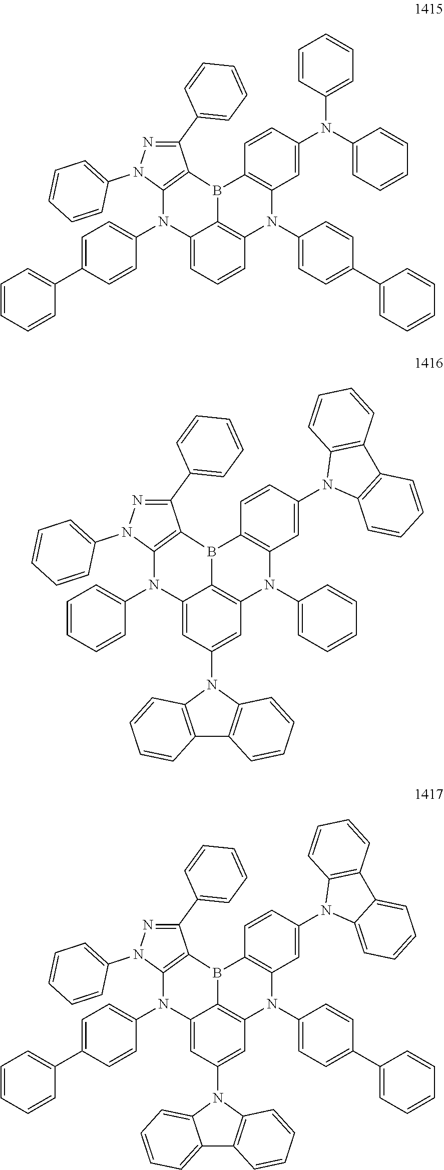 [SMILES: c1ccc(-c2ccc(N3c4cc(N(c5ccccc5)c5ccccc5)ccc4B4c5c3cccc5N(c3ccc(-c5ccccc5)cc3)c3c4c(-c4ccccc4)nn3-c3ccccc3)cc2)cc1 ; c1ccc(-c2nn(-c3ccccc3)c3c2B2c4ccc(-n5c6ccccc6c6ccccc65)cc4N(c4ccccc4)c4cc(-n5c6ccccc6c6ccccc65)cc(c42)N3c2ccccc2)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3c4c(-c4ccccc4)nn3-c3ccccc3)cc2)cc1]

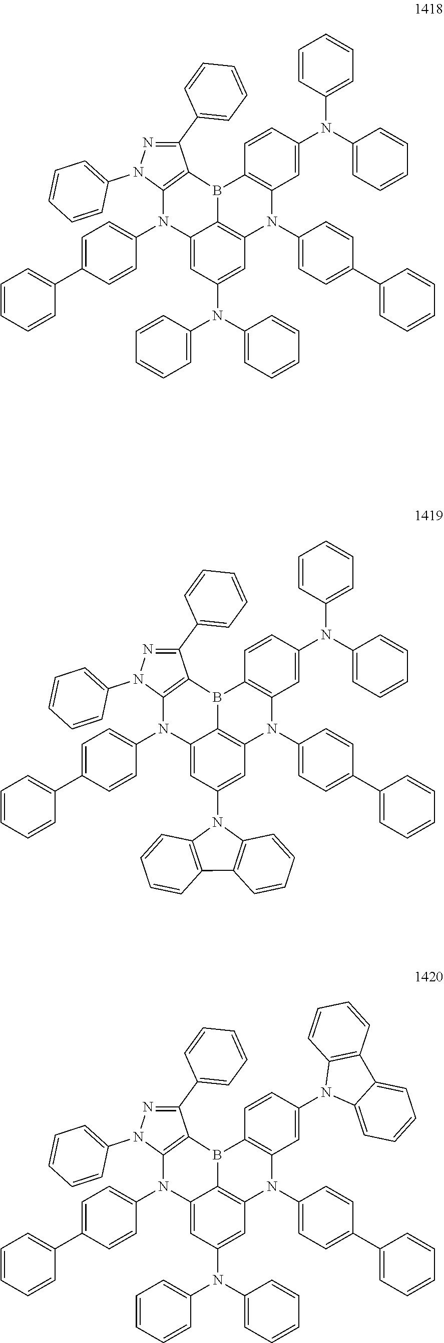 [SMILES: c1ccc(-c2ccc(N3c4cc(N(c5ccccc5)c5ccccc5)ccc4B4c5c3cc(N(c3ccccc3)c3ccccc3)cc5N(c3ccc(-c5ccccc5)cc3)c3c4c(-c4ccccc4)nn3-c3ccccc3)cc2)cc1 ; c1ccc(-c2ccc(N3c4cc(N(c5ccccc5)c5ccccc5)ccc4B4c5c3cc(-n3c6ccccc6c6ccccc63)cc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6ccccc65)ccc4B4c5c3cc(N(c3ccccc3)c3ccccc3)cc5N(c3ccc(-c5ccccc5)cc3)c3c4c(-c4ccccc4)nn3-c3ccccc3)cc2)cc1]

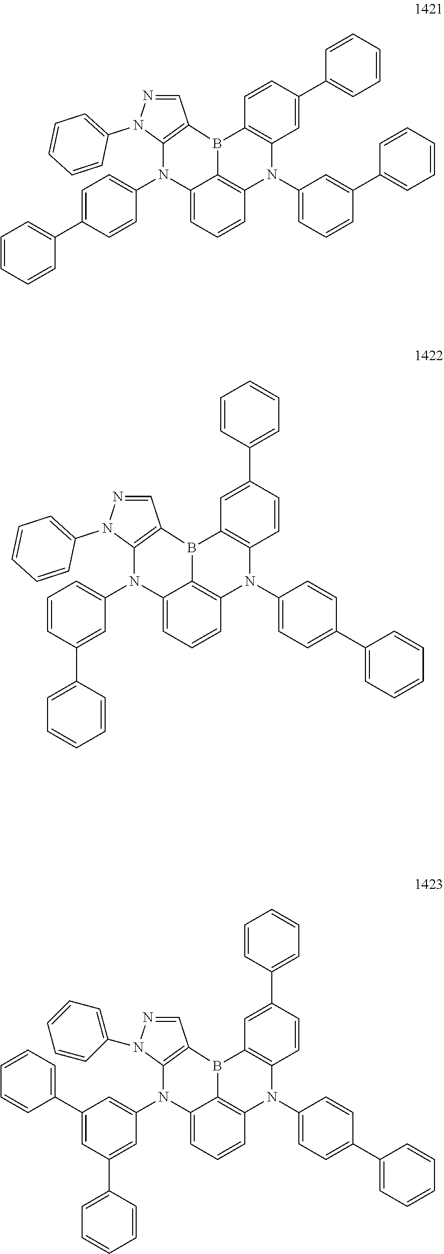 [SMILES: c1ccc(-c2ccc(N3c4cccc5c4B(c4ccc(-c6ccccc6)cc4N5c4cccc(-c5ccccc5)c4)c4cnn(-c5ccccc5)c43)cc2)cc1 ; c1ccc(-c2ccc(N3c4ccc(-c5ccccc5)cc4B4c5cnn(-c6ccccc6)c5N(c5cccc(-c6ccccc6)c5)c5cccc3c54)cc2)cc1 ; c1ccc(-c2ccc(N3c4ccc(-c5ccccc5)cc4B4c5cnn(-c6ccccc6)c5N(c5cc(-c6ccccc6)cc(-c6ccccc6)c5)c5cccc3c54)cc2)cc1]

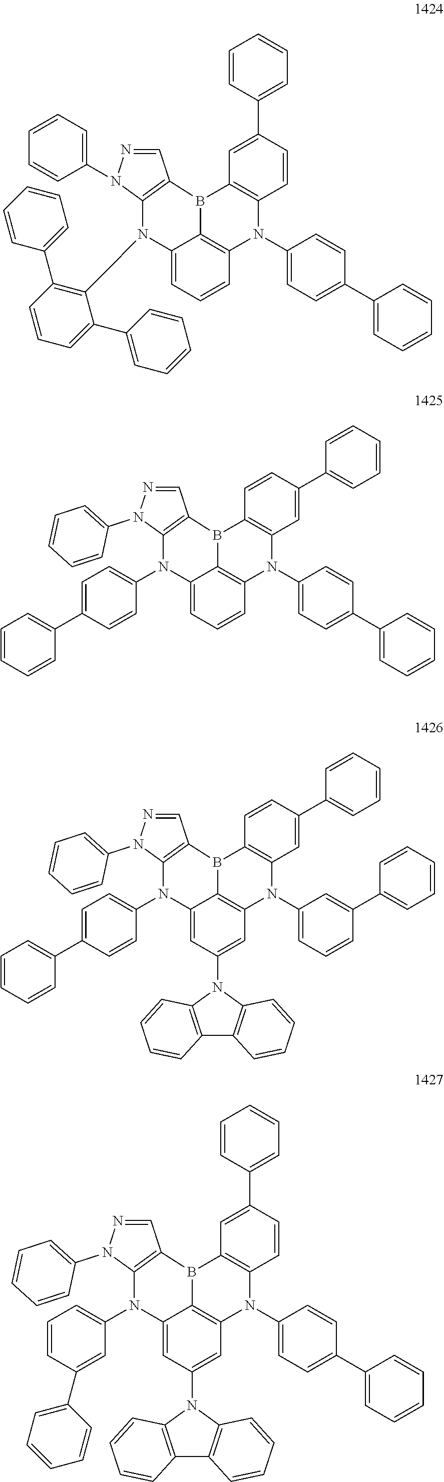

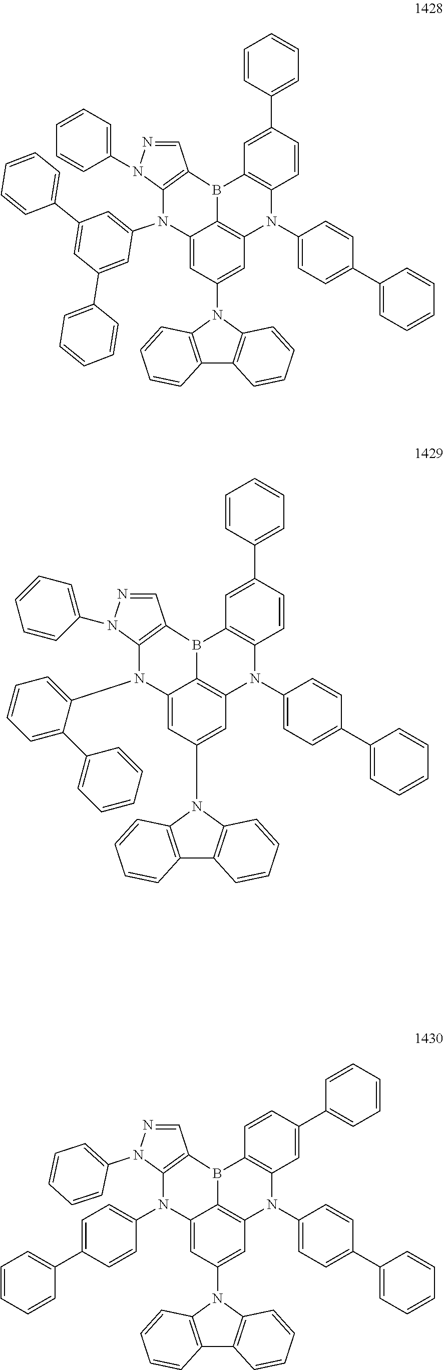 [SMILES: c1ccc(-c2ccc(N3c4ccc(-c5ccccc5)cc4B4c5cnn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4ccc(-c5ccccc5)cc4B4c5cnn(-c6ccccc6)c5N(c5ccccc5-c5ccccc5)c5cc(-n6c7ccccc7c7ccccc76)cc3c54)cc2)cc1 ; c1ccc(-c2ccc(N3c4cc(-c5ccccc5)ccc4B4c5cnn(-c6ccccc6)c5N(c5ccc(-c6ccccc6)cc5)c5cc(-n6c7ccccc7c7ccccc76)cc3c54)cc2)cc1]

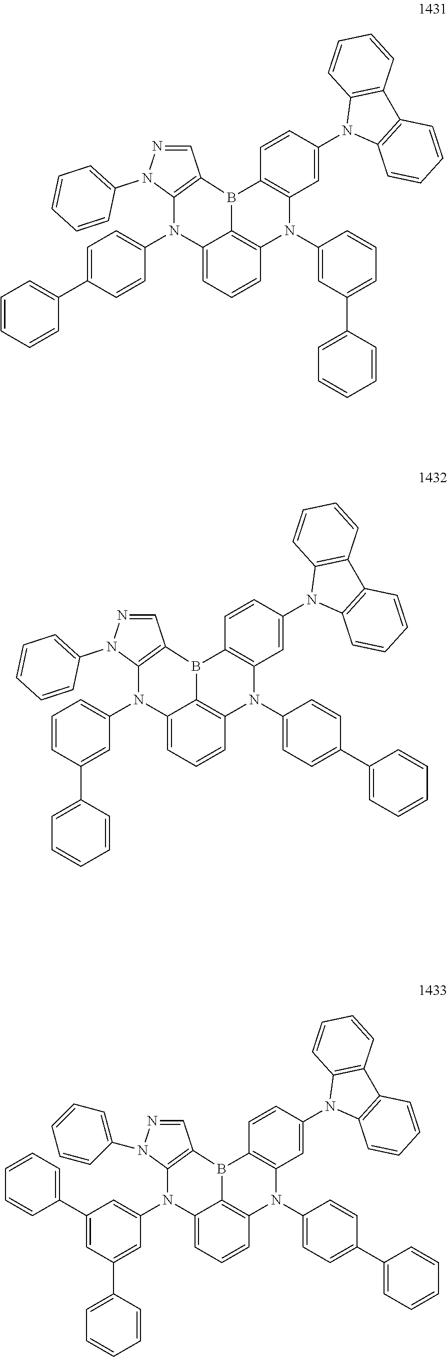 [SMILES: c1ccc(-c2ccc(N3c4cccc5c4B(c4ccc(-n6c7ccccc7c7ccccc76)cc4N5c4cccc(-c5ccccc5)c4)c4cnn(-c5ccccc5)c43)cc2)cc1 ; c1ccc(-c2ccc(N3c4cc(-n5c6ccccc6c6ccccc65)ccc4B4c5cnn(-c6ccccc6)c5N(c5cccc(-c6ccccc6)c5)c5cccc3c54)cc2)cc1 ; c1ccc(-c2ccc(N3c4cc(-n5c6ccccc6c6ccccc65)ccc4B4c5cnn(-c6ccccc6)c5N(c5cc(-c6ccccc6)cc(-c6ccccc6)c5)c5cccc3c54)cc2)cc1]

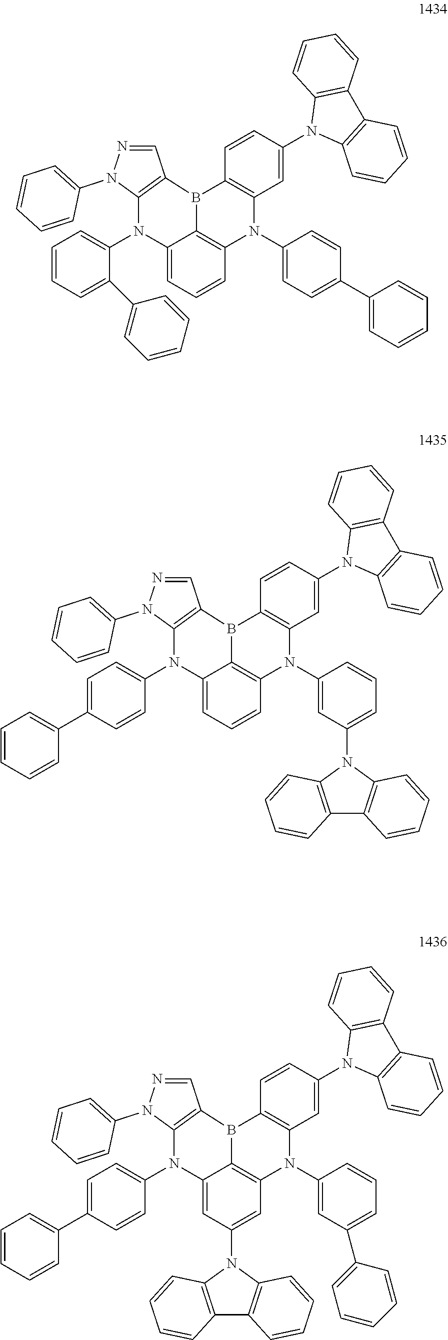 [SMILES: c1ccc(-c2ccc(N3c4cc(-n5c6ccccc6c6ccccc65)ccc4B4c5cnn(-c6ccccc6)c5N(c5ccccc5-c5ccccc5)c5cccc3c54)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4cnn(-c5ccccc5)c43)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4cnn(-c5ccccc5)c43)cc2)cc1]

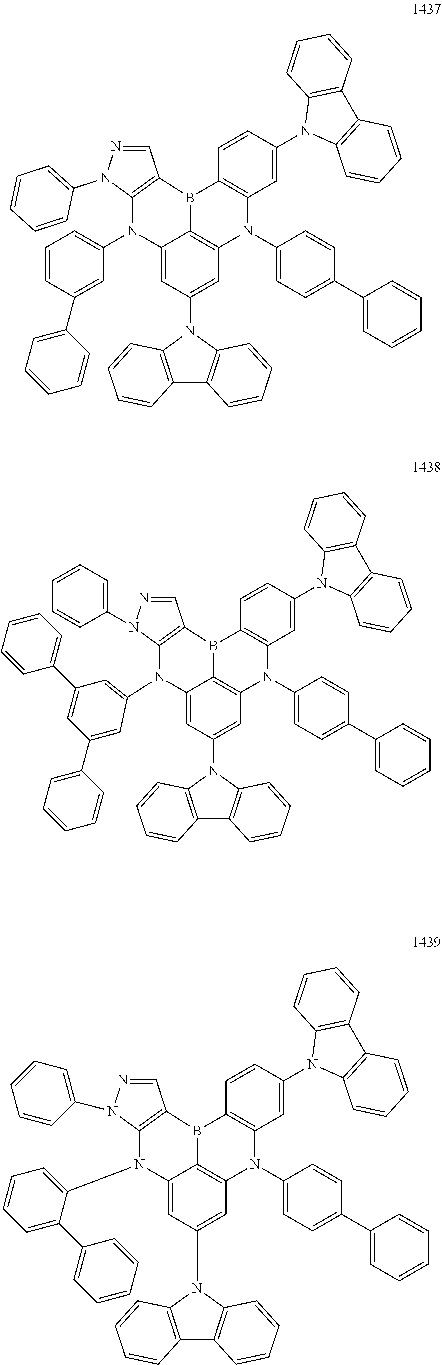 [SMILES: c1ccc(-c2ccc(N3c4cc(-n5c6ccccc6c6ccccc65)ccc4B4c5cnn(-c6ccccc6)c5N(c5cccc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5cnn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5cnn(-c6ccccc6)c5N(c5ccccc5-c5ccccc5)c5cc(-n6c7ccccc7c7ccccc76)cc3c54)cc2)cc1]

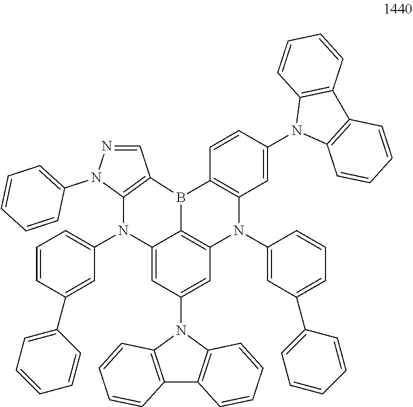 [SMILES: c1ccc(-c2cccc(N3c4cc(-n5c6ccccc6c6ccccc65)ccc4B4c5cnn(-c6ccccc6)c5N(c5cccc(-c6ccccc6)c5)c5cc(-n6c7ccccc7c7ccccc76)cc3c54)c2)cc1]

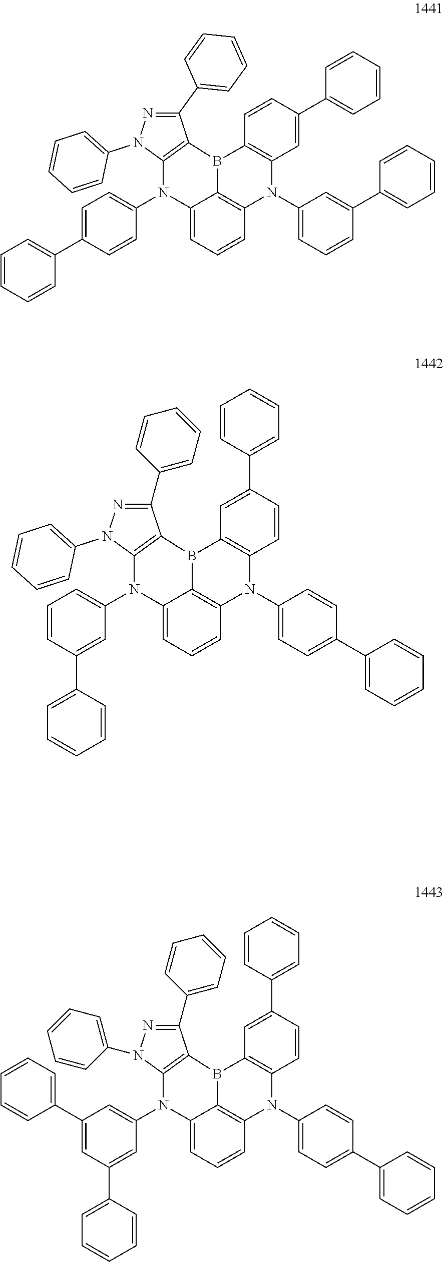 [SMILES: c1ccc(-c2ccc(N3c4cccc5c4B(c4ccc(-c6ccccc6)cc4N5c4cccc(-c5ccccc5)c4)c4c(-c5ccccc5)nn(-c5ccccc5)c43)cc2)cc1 ; c1ccc(-c2ccc(N3c4ccc(-c5ccccc5)cc4B4c5c3cccc5N(c3cccc(-c5ccccc5)c3)c3c4c(-c4ccccc4)nn3-c3ccccc3)cc2)cc1 ; c1ccc(-c2ccc(N3c4ccc(-c5ccccc5)cc4B4c5c3cccc5N(c3cc(-c5ccccc5)cc(-c5ccccc5)c3)c3c4c(-c4ccccc4)nn3-c3ccccc3)cc2)cc1]

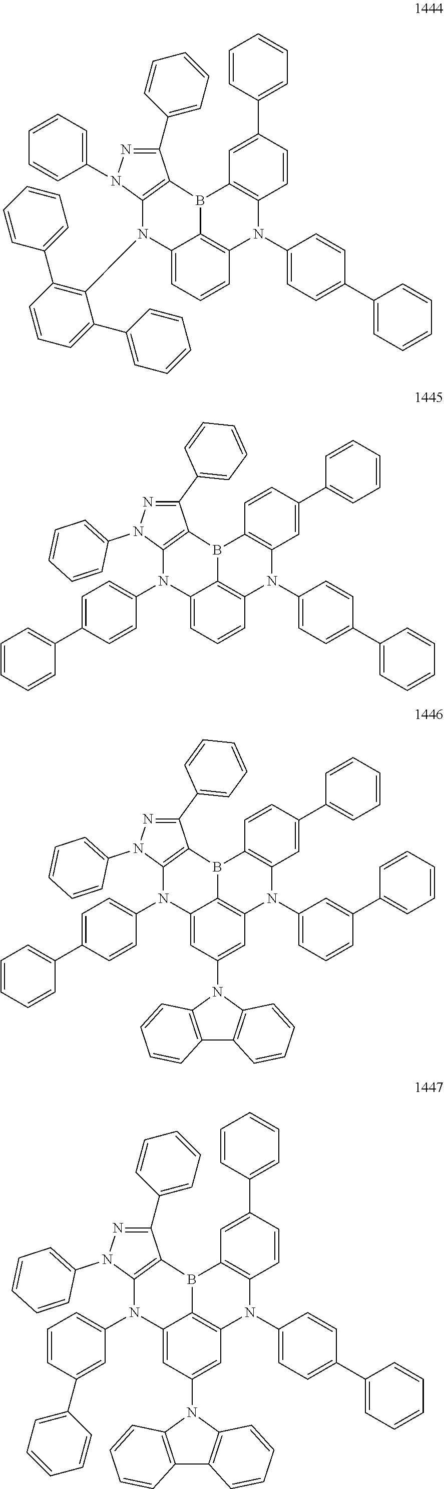 [SMILES: c1ccc(-c2ccc(N3c4ccc(-c5ccccc5)cc4B4c5c3cccc5N(c3c(-c5ccccc5)cccc3-c3ccccc3)c3c4c(-c4ccccc4)nn3-c3ccccc3)cc2)cc1 ; c1ccc(-c2ccc(N3c4cc(-c5ccccc5)ccc4B4c5c3cccc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6ccccc65)cc5c4B(c4ccc(-c6ccccc6)cc4N5c4cccc(-c5ccccc5)c4)c4c(-c5ccccc5)nn(-c5ccccc5)c43)cc2)cc1 ; c1ccc(-c2ccc(N3c4ccc(-c5ccccc5)cc4B4c5c3cc(-n3c6ccccc6c6ccccc63)cc5N(c3cccc(-c5ccccc5)c3)c3c4c(-c4ccccc4)nn3-c3ccccc3)cc2)cc1]

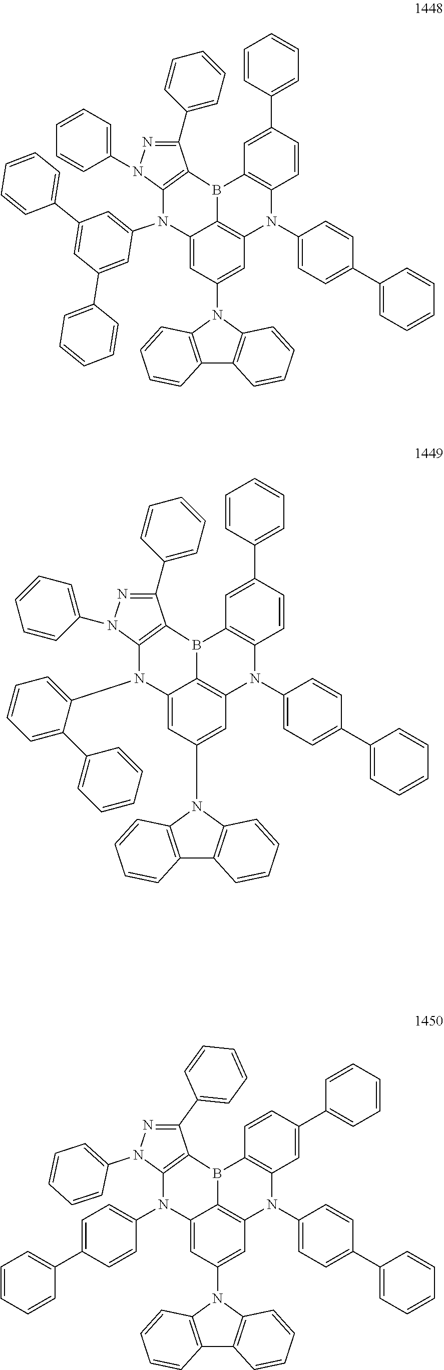 [SMILES: c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3c4c(-c4ccccc4)nn3-c3ccccc3)cc2)cc1 ; c1ccc(-c2ccc(N3c4ccc(-c5ccccc5)cc4B4c5c3cc(-n3c6ccccc6c6ccccc63)cc5N(c3ccccc3-c3ccccc3)c3c4c(-c4ccccc4)nn3-c3ccccc3)cc2)cc1 ; c1ccc(-c2ccc(N3c4cc(-c5ccccc5)ccc4B4c5c3cc(-n3c6ccccc6c6ccccc63)cc5N(c3ccc(-c5ccccc5)cc3)c3c4c(-c4ccccc4)nn3-c3ccccc3)cc2)cc1]

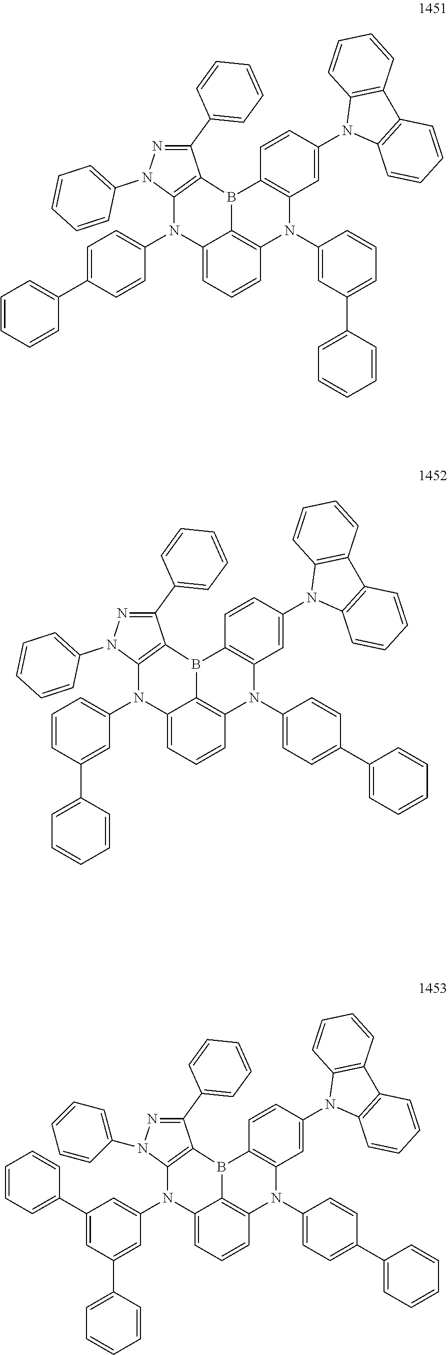 [SMILES: c1ccc(-c2ccc(N3c4cccc5c4B(c4ccc(-n6c7ccccc7c7ccccc76)cc4N5c4cccc(-c5ccccc5)c4)c4c(-c5ccccc5)nn(-c5ccccc5)c43)cc2)cc1 ; c1ccc(-c2ccc(N3c4cc(-n5c6ccccc6c6ccccc65)ccc4B4c5c3cccc5N(c3cccc(-c5ccccc5)c3)c3c4c(-c4ccccc4)nn3-c3ccccc3)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3c4c(-c4ccccc4)nn3-c3ccccc3)cc2)cc1]

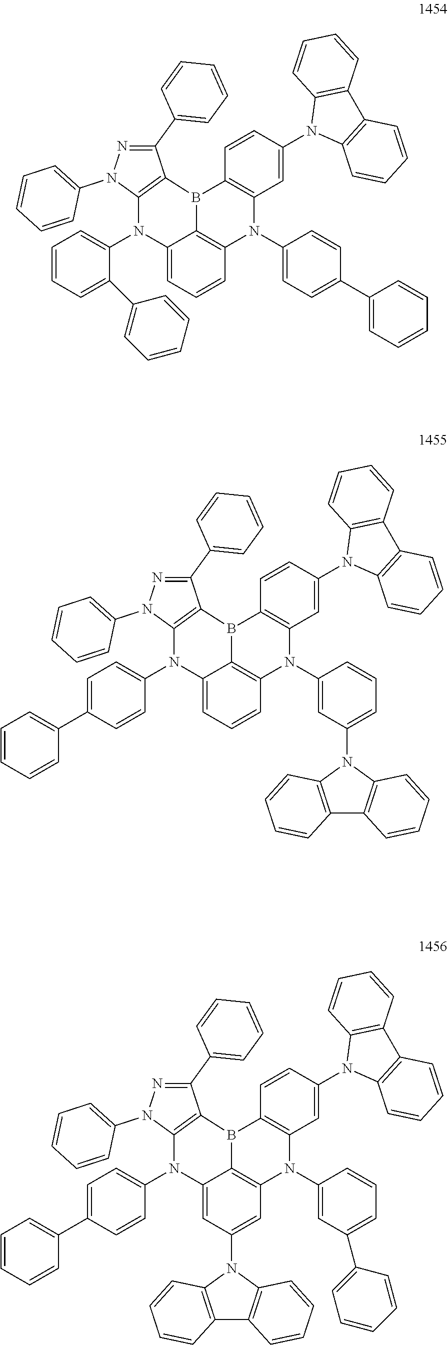 [SMILES: c1ccc(-c2ccc(N3c4cc(-n5c6ccccc6c6ccccc65)ccc4B4c5c3cccc5N(c3ccccc3-c3ccccc3)c3c4c(-c4ccccc4)nn3-c3ccccc3)cc2)cc1 ; c1ccc(-c2ccc(N3c4cccc5c4B(c4ccc(-n6c7ccccc7c7ccccc76)cc4N5c4cccc(-n5c6ccccc6c6ccccc65)c4)c4c(-c5ccccc5)nn(-c5ccccc5)c43)cc2)cc1 ; c1ccc(-c2ccc(N3c4cc(-n5c6ccccc6c6ccccc65)cc5c4B(c4ccc(-n6c7ccccc7c7ccccc76)cc4N5c4cccc(-c5ccccc5)c4)c4c(-c5ccccc5)nn(-c5ccccc5)c43)cc2)cc1]

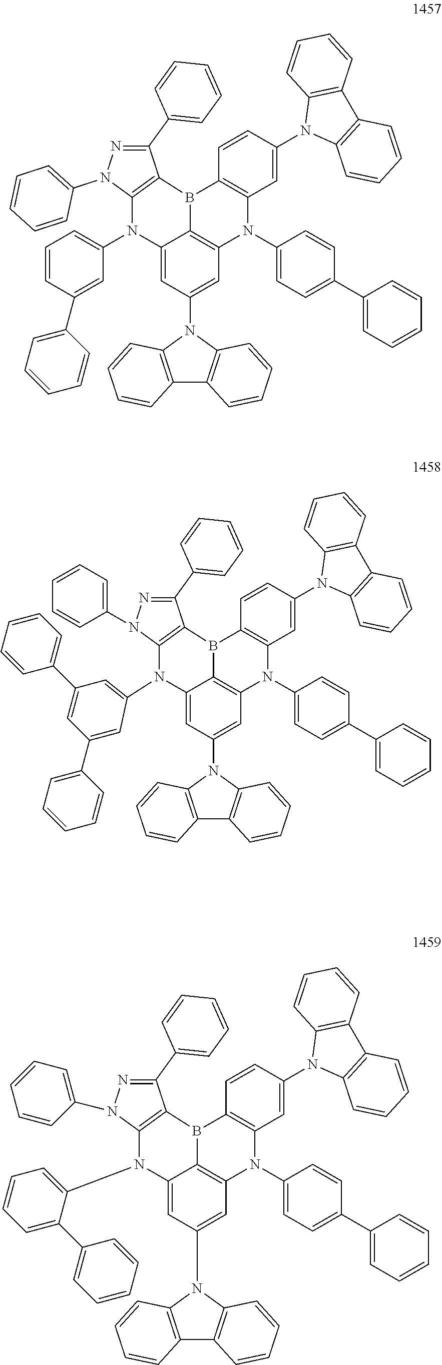 [SMILES: c1ccc(-c2ccc(N3c4cc(-n5c6ccccc6c6ccccc65)ccc4B4c5c3cc(-n3c6ccccc6c6ccccc63)cc5N(c3cccc(-c5ccccc5)c3)c3c4c(-c4ccccc4)nn3-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3cc(-c5ccccc5)cc(-c5ccccc5)c3)c3c4c(-c4ccccc4)nn3-c3ccccc3)cc2)cc1 ; c1ccc(-c2ccc(N3c4cc(-n5c6ccccc6c6ccccc65)ccc4B4c5c3cc(-n3c6ccccc6c6ccccc63)cc5N(c3ccccc3-c3ccccc3)c3c4c(-c4ccccc4)nn3-c3ccccc3)cc2)cc1]

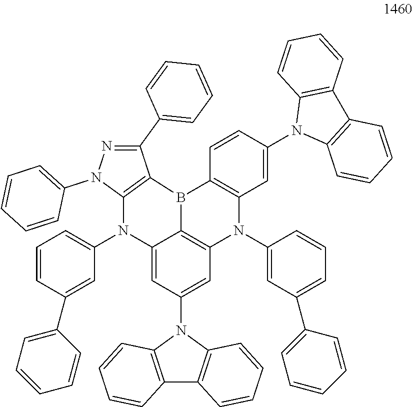 [SMILES: c1ccc(-c2cccc(N3c4cc(-n5c6ccccc6c6ccccc65)ccc4B4c5c3cc(-n3c6ccccc6c6ccccc63)cc5N(c3cccc(-c5ccccc5)c3)c3c4c(-c4ccccc4)nn3-c3ccccc3)c2)cc1]

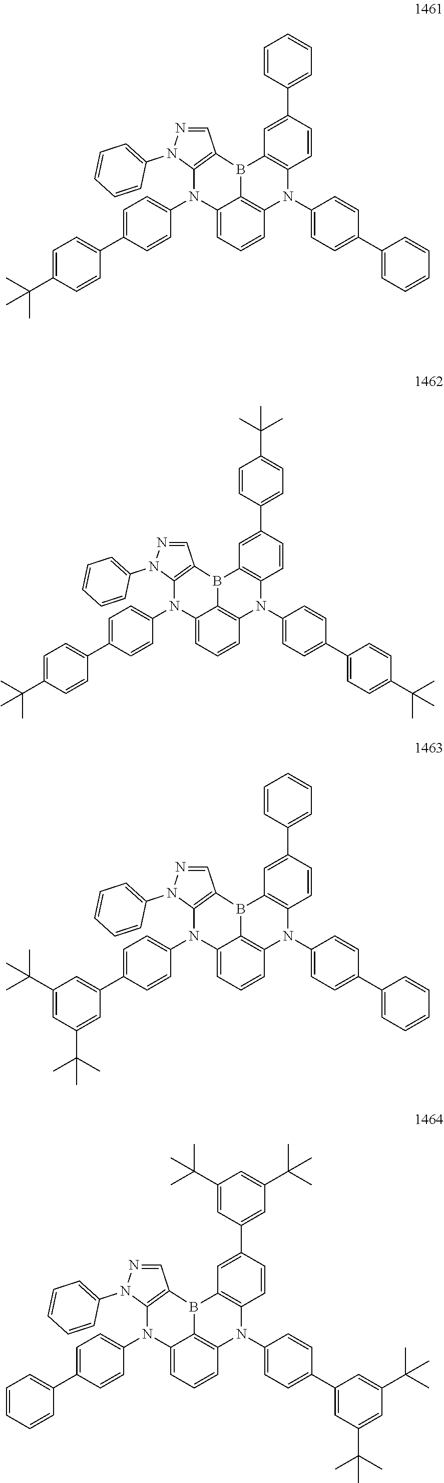

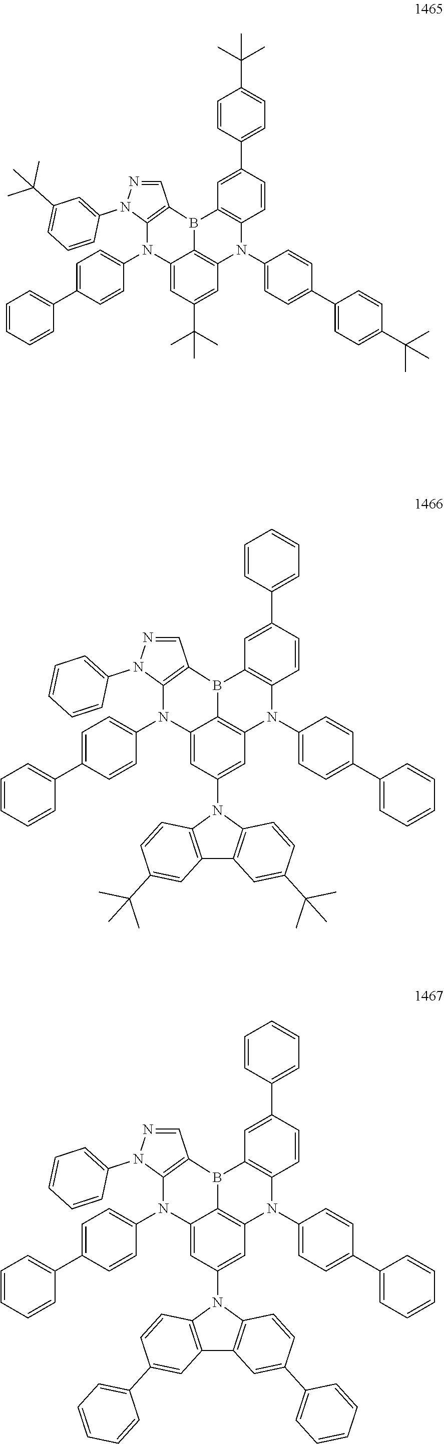 [SMILES: CC(C)(C)c1ccc(-c2ccc(N3c4ccc(-c5ccc(C(C)(C)C)cc5)cc4B4c5cnn(-c6cccc(C(C)(C)C)c6)c5N(c5ccc(-c6ccccc6)cc5)c5cc(C(C)(C)C)cc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(cnn1-c1ccccc1)B3c1cc(-c3ccccc3)ccc1N2c1ccc(-c2ccccc2)cc1 ; c1ccc(-c2ccc(N3c4ccc(-c5ccccc5)cc4B4c5cnn(-c6ccccc6)c5N(c5ccc(-c6ccccc6)cc5)c5cc(-n6c7ccc(-c8ccccc8)cc7c7cc(-c8ccccc8)ccc76)cc3c54)cc2)cc1]

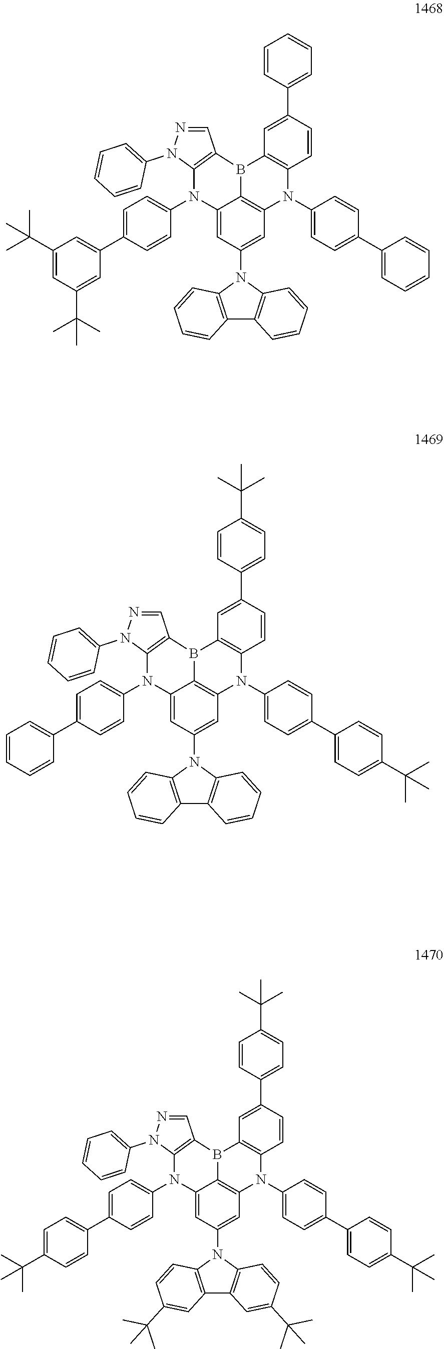

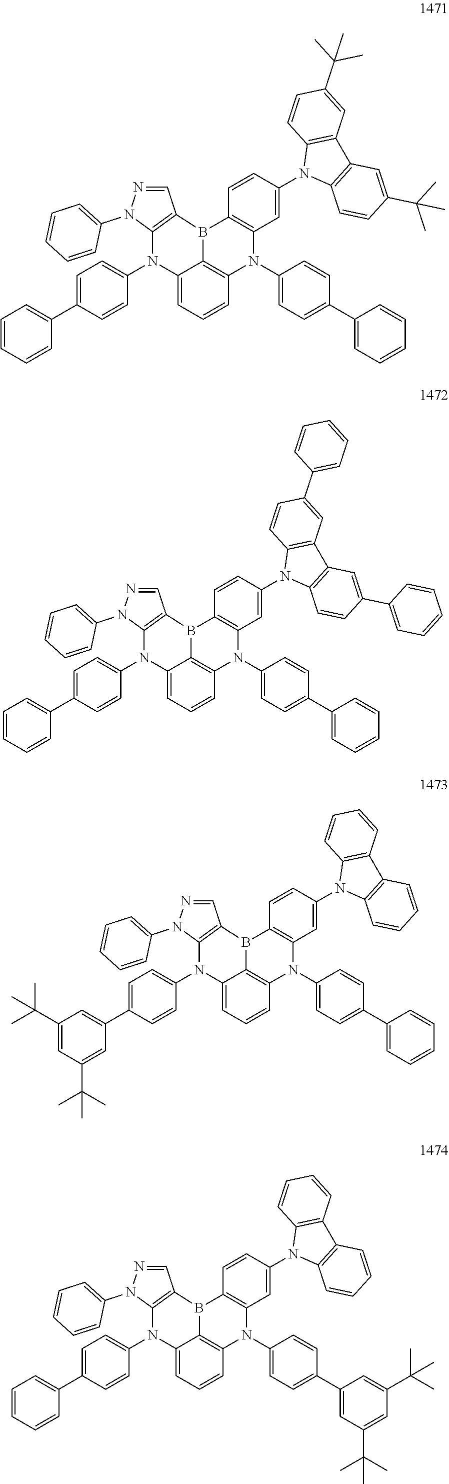

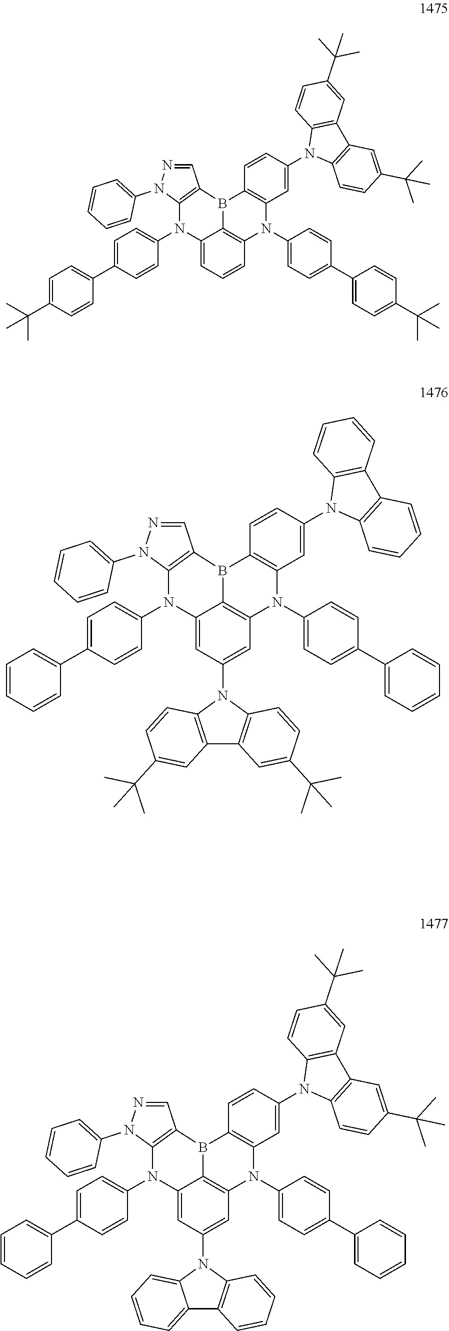 [SMILES: CC(C)(C)c1ccc(-c2ccc(N3c4cc(-n5c6ccc(C(C)(C)C)cc6c6cc(C(C)(C)C)ccc65)ccc4B4c5cnn(-c6ccccc6)c5N(c5ccc(-c6ccc(C(C)(C)C)cc6)cc5)c5cccc3c54)cc2)cc1 ; CC(C)(C)c1ccc2c(c1)c1cc(C(C)(C)C)ccc1n2-c1cc2c3c(c1)N(c1ccc(-c4ccccc4)cc1)c1c(cnn1-c1ccccc1)B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1cnn(-c2ccccc2)c1N3c1ccc(-c2ccccc2)cc1]

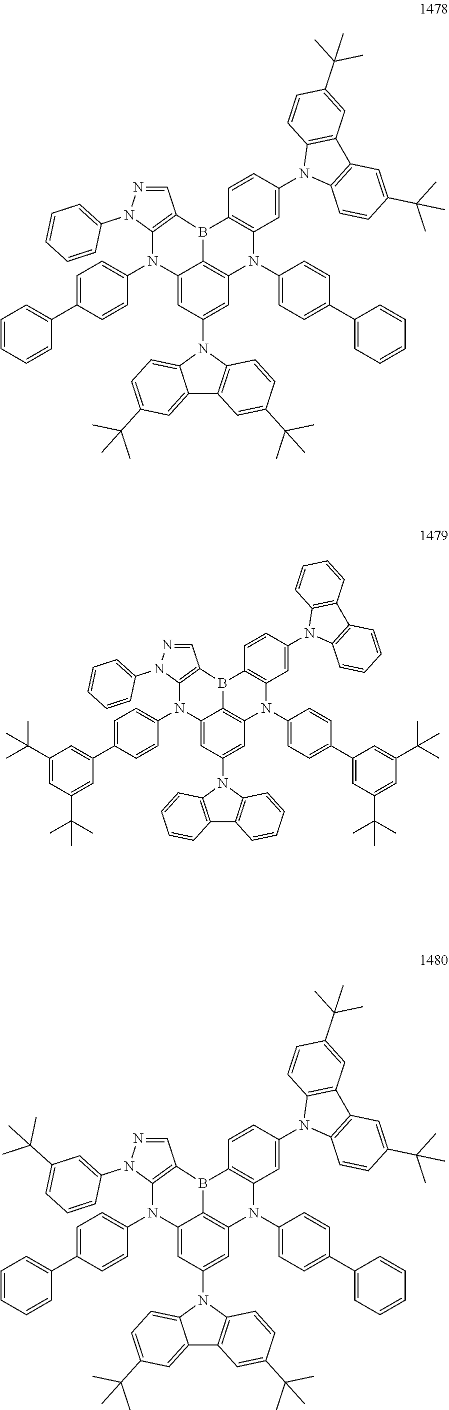

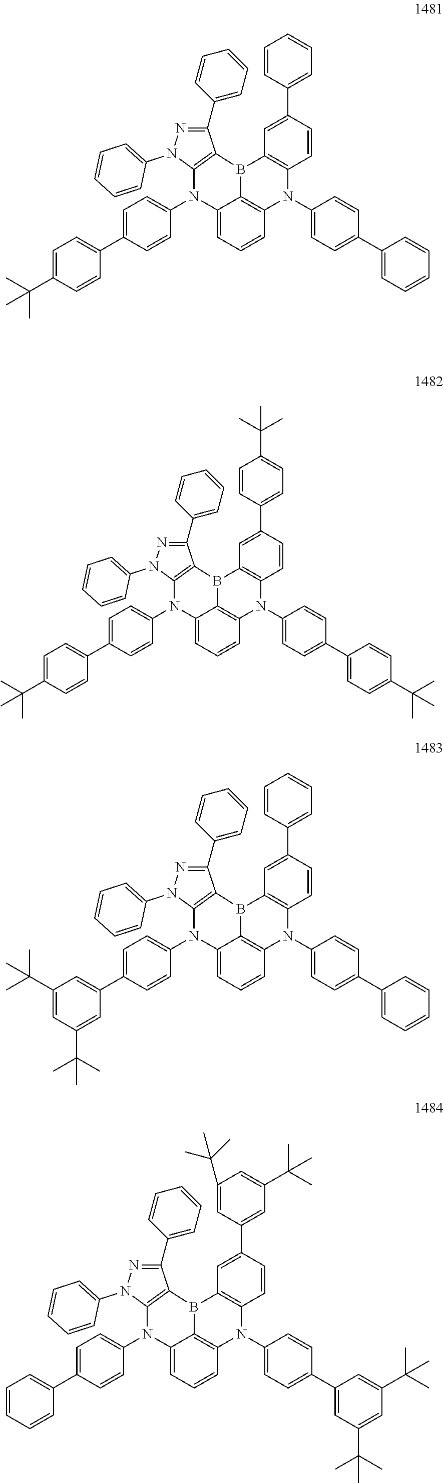

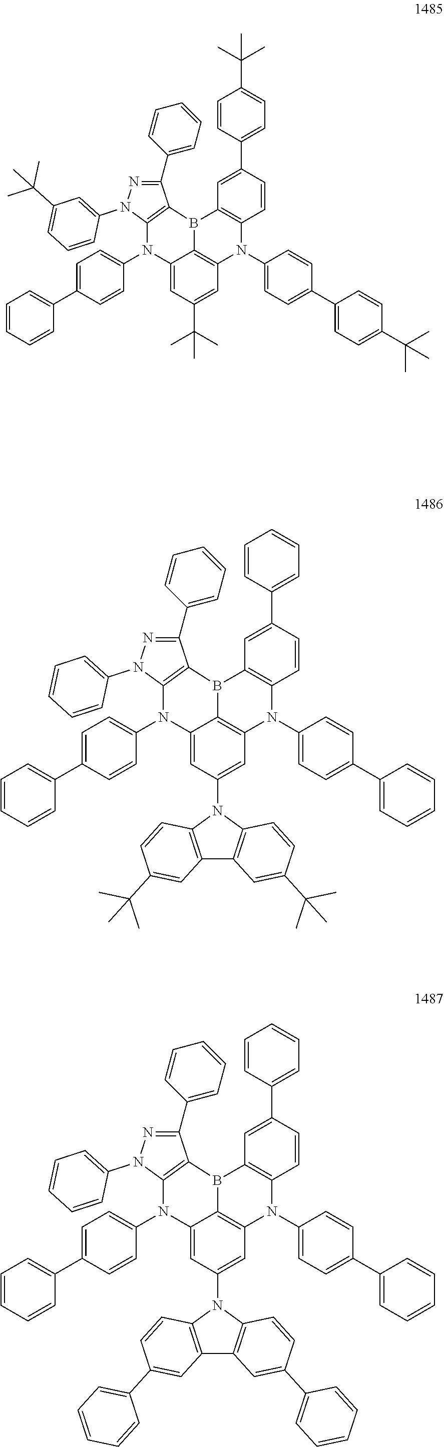 [SMILES: CC(C)(C)c1ccc(-c2ccc(N3c4ccc(-c5ccc(C(C)(C)C)cc5)cc4B4c5c3cc(C(C)(C)C)cc5N(c3ccc(-c5ccccc5)cc3)c3c4c(-c4ccccc4)nn3-c3cccc(C(C)(C)C)c3)cc2)cc1 ; CC(C)(C)c1ccc2c(c1)c1cc(C(C)(C)C)ccc1n2-c1cc2c3c(c1)N(c1ccc(-c4ccccc4)cc1)c1c(c(-c4ccccc4)nn1-c1ccccc1)B3c1cc(-c3ccccc3)ccc1N2c1ccc(-c2ccccc2)cc1 ; c1ccc(-c2ccc(N3c4ccc(-c5ccccc5)cc4B4c5c3cc(-n3c6ccc(-c7ccccc7)cc6c6cc(-c7ccccc7)ccc63)cc5N(c3ccc(-c5ccccc5)cc3)c3c4c(-c4ccccc4)nn3-c3ccccc3)cc2)cc1]

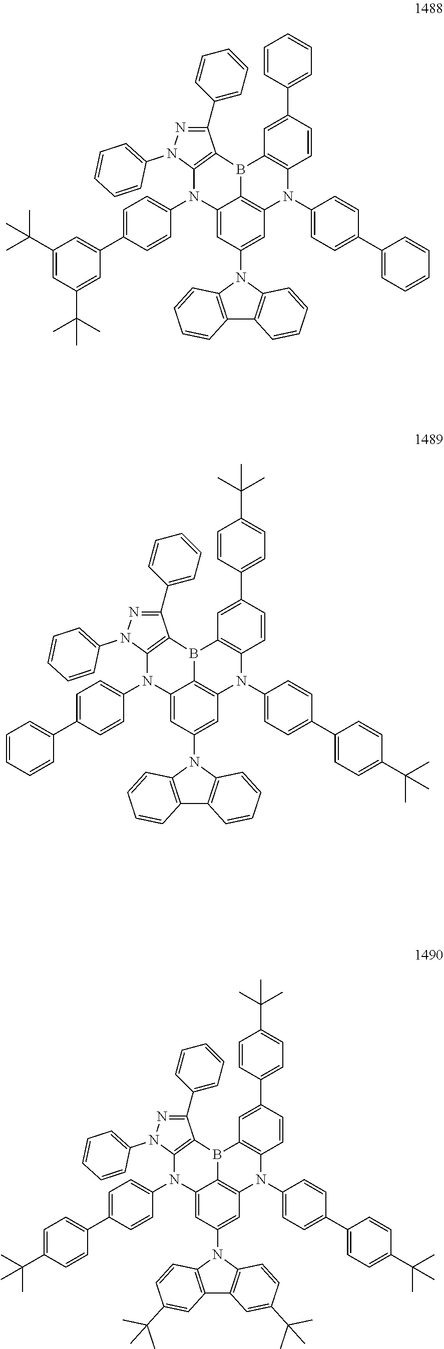

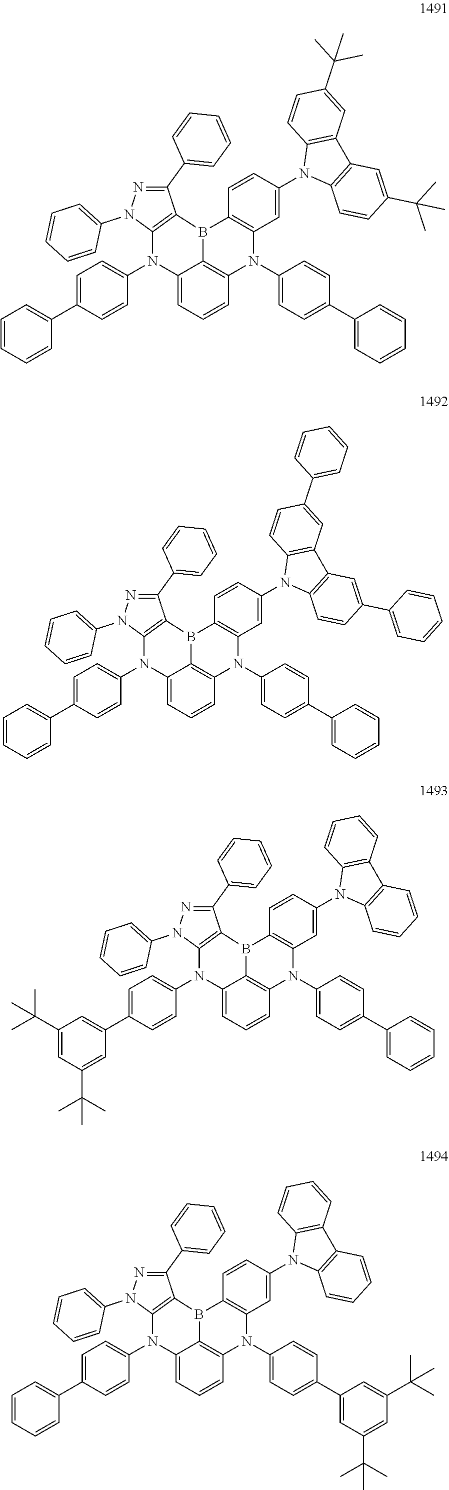

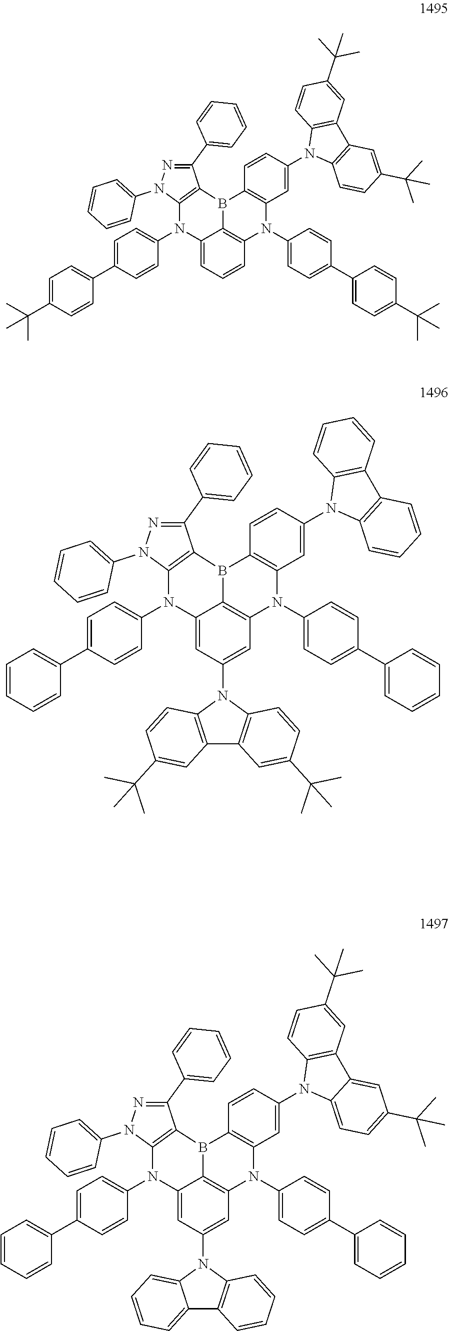 [SMILES: CC(C)(C)c1ccc(-c2ccc(N3c4cc(-n5c6ccc(C(C)(C)C)cc6c6cc(C(C)(C)C)ccc65)ccc4B4c5c3cccc5N(c3ccc(-c5ccc(C(C)(C)C)cc5)cc3)c3c4c(-c4ccccc4)nn3-c3ccccc3)cc2)cc1 ; CC(C)(C)c1ccc2c(c1)c1cc(C(C)(C)C)ccc1n2-c1cc2c3c(c1)N(c1ccc(-c4ccccc4)cc1)c1c(c(-c4ccccc4)nn1-c1ccccc1)B3c1ccc(-n3c4ccccc4c4ccccc43)cc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(-c2ccccc2)nn(-c2ccccc2)c1N3c1ccc(-c2ccccc2)cc1]

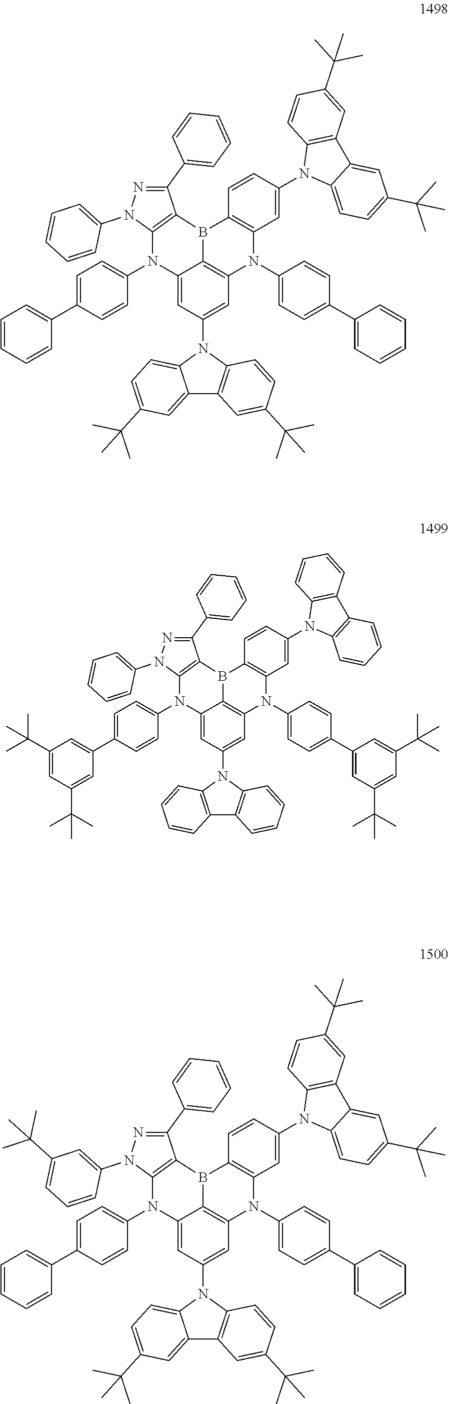

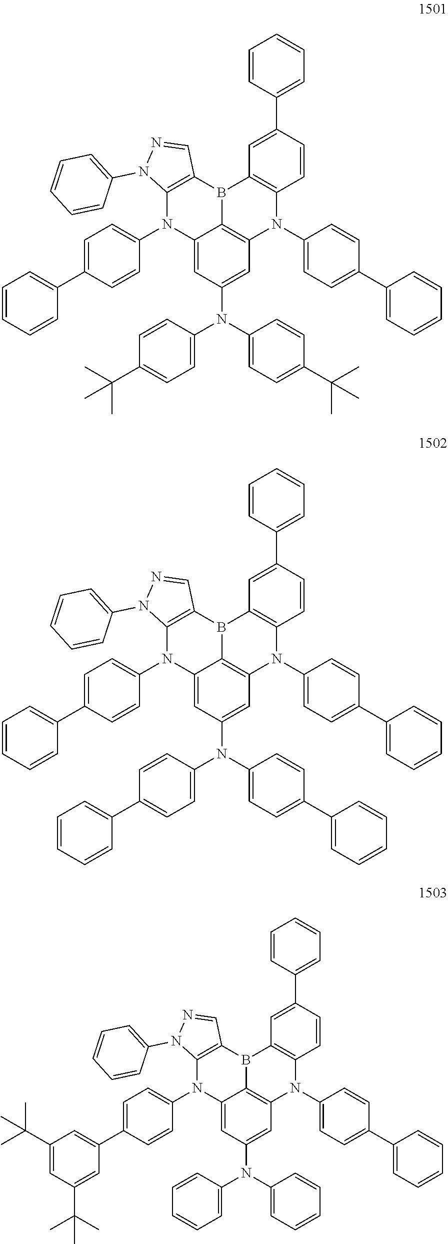 [SMILES: CC(C)(C)c1ccc(N(c2ccc(C(C)(C)C)cc2)c2cc3c4c(c2)N(c2ccc(-c5ccccc5)cc2)c2c(cnn2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(cnn3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4cnn(-c5ccccc5)c43)cc2)cc(C(C)(C)C)c1]

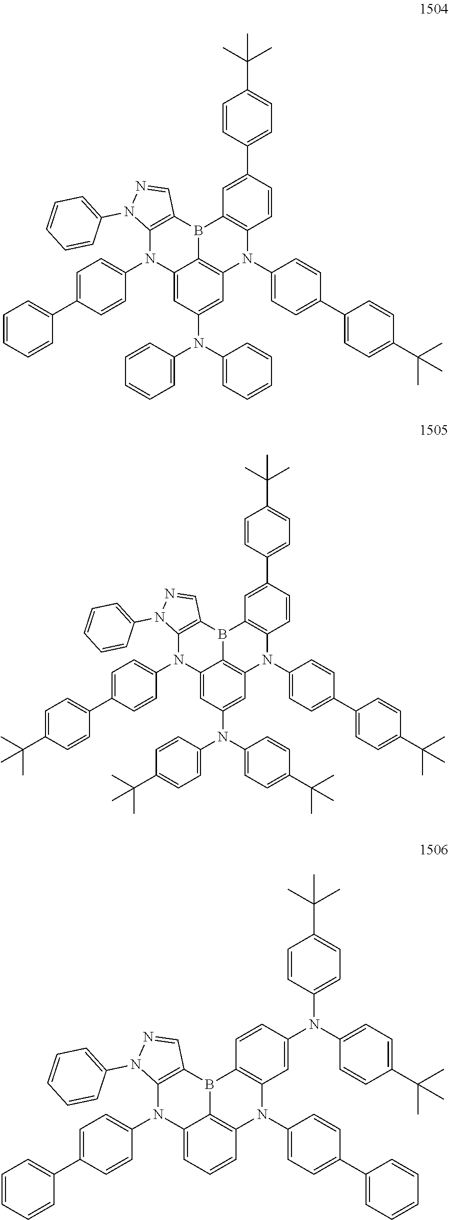

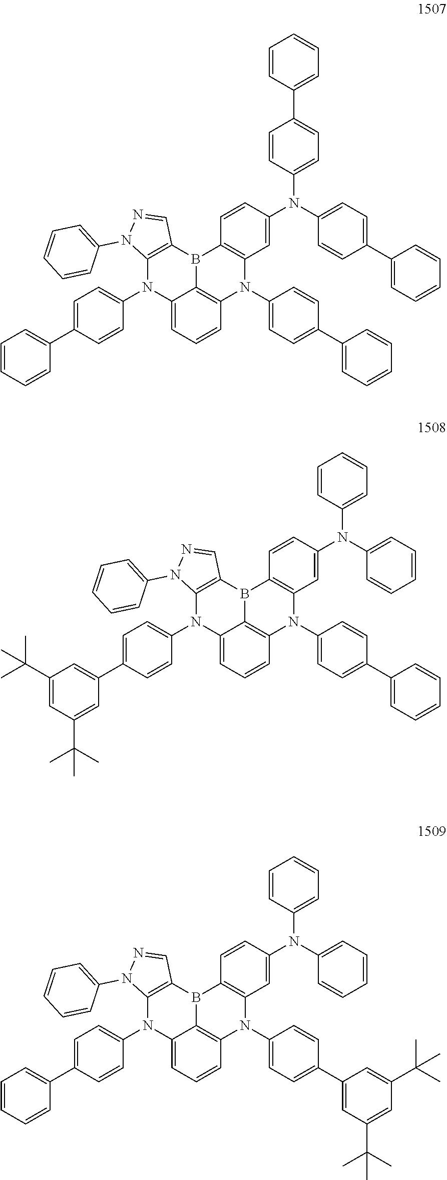 [SMILES: c1ccc(-c2ccc(N(c3ccc(-c4ccccc4)cc3)c3ccc4c(c3)N(c3ccc(-c5ccccc5)cc3)c3cccc5c3B4c3cnn(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4cnn(-c5ccccc5)c43)cc2)cc(C(C)(C)C)c1 ; CC(C)(C)c1cc(-c2ccc(N3c4cc(N(c5ccccc5)c5ccccc5)ccc4B4c5cnn(-c6ccccc6)c5N(c5ccc(-c6ccccc6)cc5)c5cccc3c54)cc2)cc(C(C)(C)C)c1]

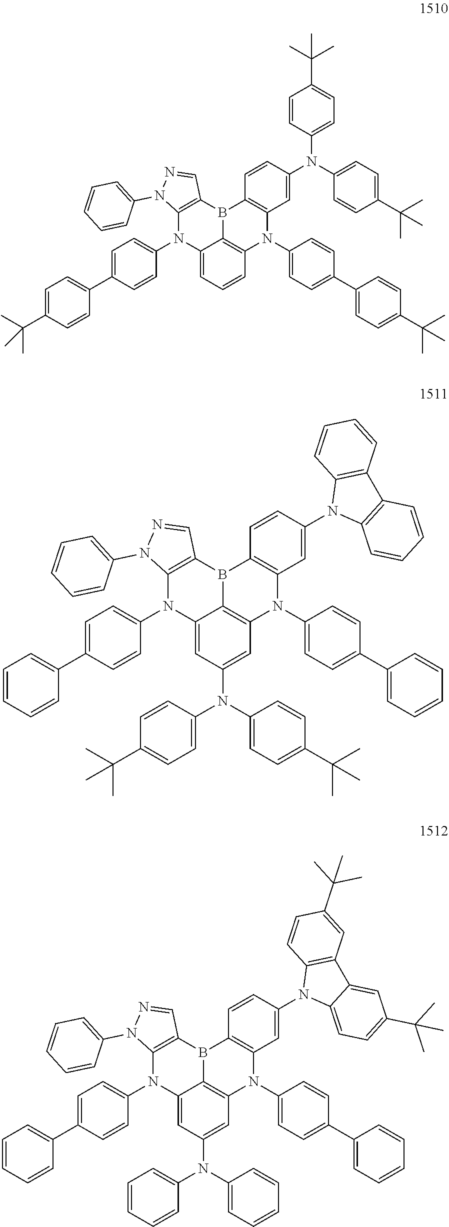 [SMILES: CC(C)(C)c1ccc(-c2ccc(N3c4cc(N(c5ccc(C(C)(C)C)cc5)c5ccc(C(C)(C)C)cc5)ccc4B4c5cnn(-c6ccccc6)c5N(c5ccc(-c6ccc(C(C)(C)C)cc6)cc5)c5cccc3c54)cc2)cc1 ; CC(C)(C)c1ccc(N(c2ccc(C(C)(C)C)cc2)c2cc3c4c(c2)N(c2ccc(-c5ccccc5)cc2)c2c(cnn2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1cnn(-c2ccccc2)c1N3c1ccc(-c2ccccc2)cc1]

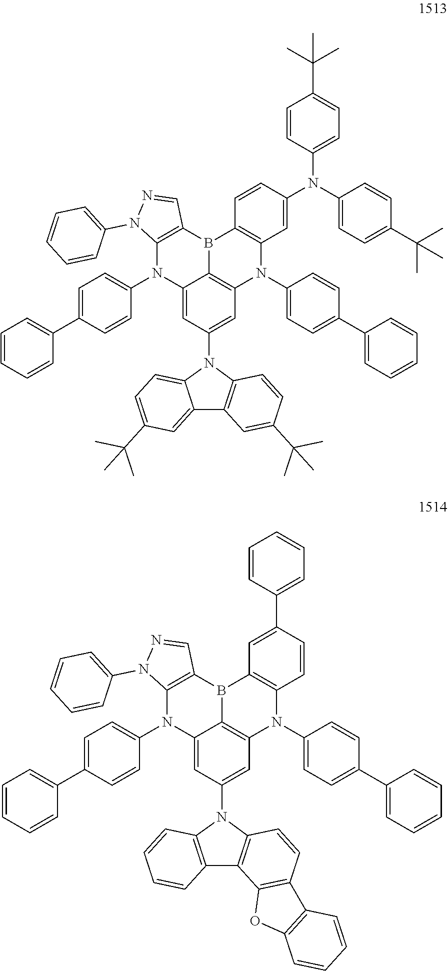 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2cnn(-c3ccccc3)c2N4c2ccc(-c3ccccc3)cc2)cc1 ; c1ccc(-c2ccc(N3c4ccc(-c5ccccc5)cc4B4c5cnn(-c6ccccc6)c5N(c5ccc(-c6ccccc6)cc5)c5cc(-n6c7ccccc7c7c8oc9ccccc9c8ccc76)cc3c54)cc2)cc1]

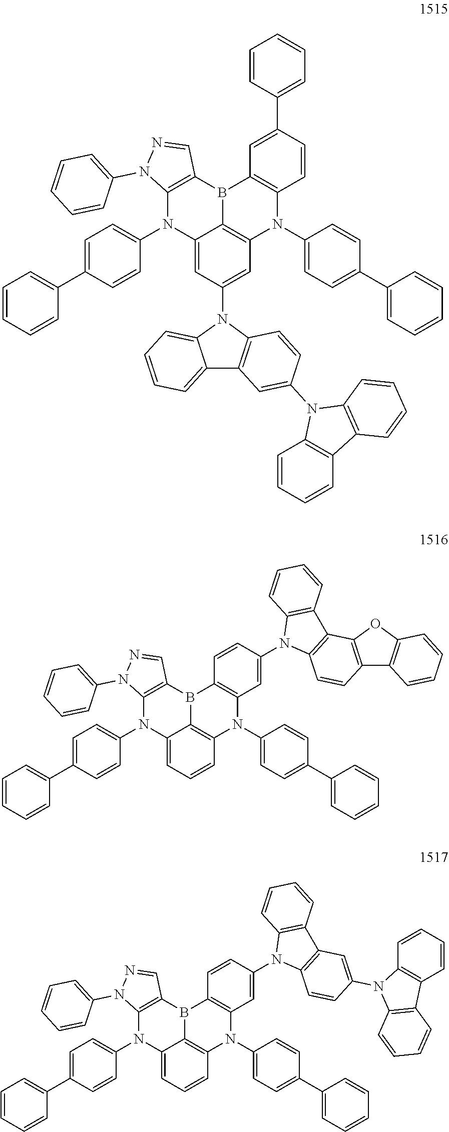 [SMILES: c1ccc(-c2ccc(N3c4ccc(-c5ccccc5)cc4B4c5cnn(-c6ccccc6)c5N(c5ccc(-c6ccccc6)cc5)c5cc(-n6c7ccccc7c7cc(-n8c9ccccc9c9ccccc98)ccc76)cc3c54)cc2)cc1 ; c1ccc(-c2ccc(N3c4cc(-n5c6ccccc6c6c7oc8ccccc8c7ccc65)ccc4B4c5cnn(-c6ccccc6)c5N(c5ccc(-c6ccccc6)cc5)c5cccc3c54)cc2)cc1 ; c1ccc(-c2ccc(N3c4cc(-n5c6ccccc6c6cc(-n7c8ccccc8c8ccccc87)ccc65)ccc4B4c5cnn(-c6ccccc6)c5N(c5ccc(-c6ccccc6)cc5)c5cccc3c54)cc2)cc1]

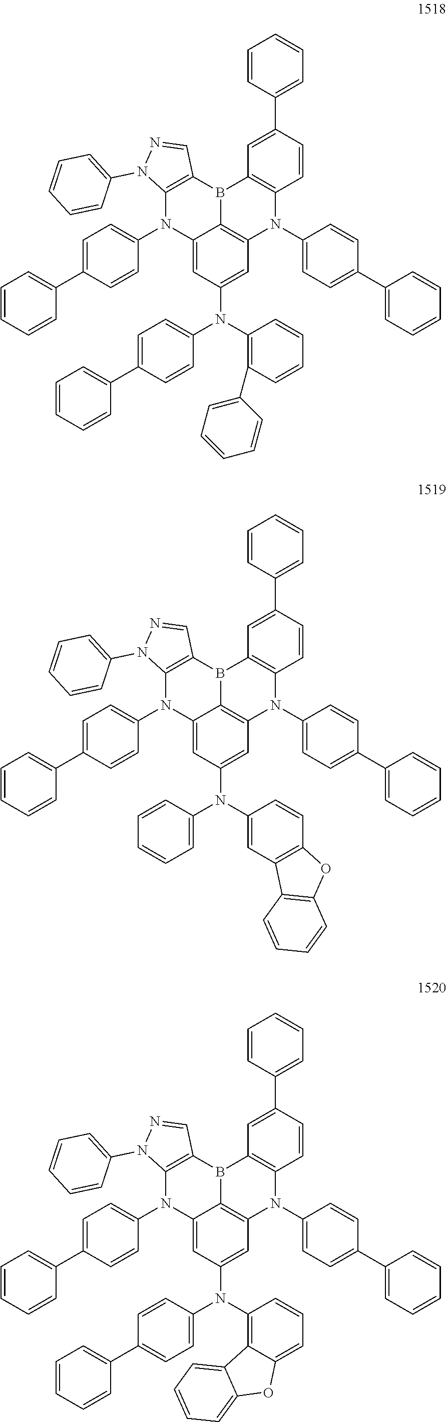 [SMILES: c1ccc(-c2ccc(N(c3cc4c5c(c3)N(c3ccc(-c6ccccc6)cc3)c3c(cnn3-c3ccccc3)B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5cnn(-c6ccccc6)c5N(c5ccc(-c6ccccc6)cc5)c5cc(N(c6ccccc6)c6ccc7oc8ccccc8c7c6)cc3c54)cc2)cc1 ; c1ccc(-c2ccc(N3c4ccc(-c5ccccc5)cc4B4c5cnn(-c6ccccc6)c5N(c5ccc(-c6ccccc6)cc5)c5cc(N(c6ccc(-c7ccccc7)cc6)c6cccc7oc8ccccc8c67)cc3c54)cc2)cc1]

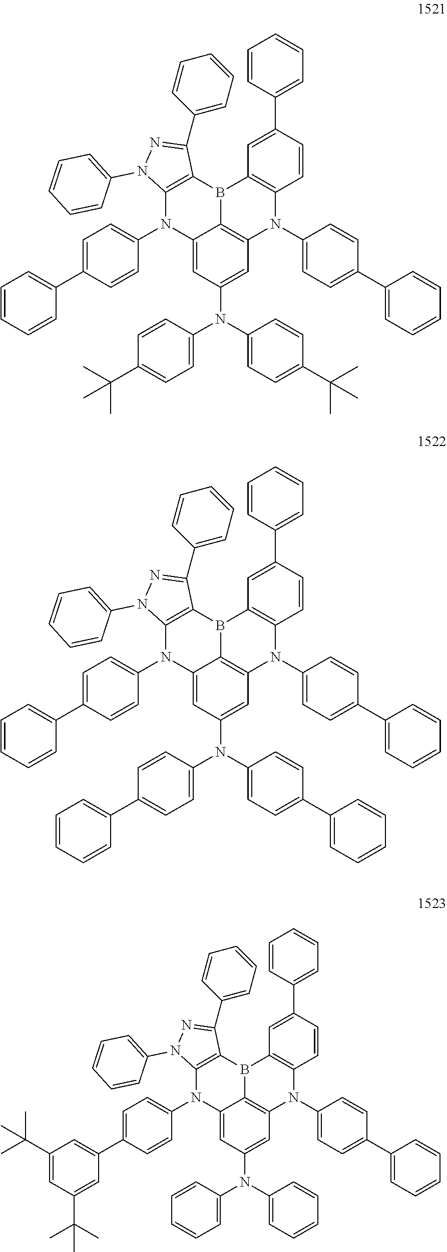 [SMILES: CC(C)(C)c1ccc(N(c2ccc(C(C)(C)C)cc2)c2cc3c4c(c2)N(c2ccc(-c5ccccc5)cc2)c2c(c(-c5ccccc5)nn2-c2ccccc2)B4c2cc(-c4ccccc4)ccc2N3c2ccc(-c3ccccc3)cc2)cc1 ; c1ccc(-c2ccc(N(c3ccc(-c4ccccc4)cc3)c3cc4c5c(c3)N(c3ccc(-c6ccccc6)cc3)c3c(c(-c6ccccc6)nn3-c3ccccc3)B5c3cc(-c5ccccc5)ccc3N4c3ccc(-c4ccccc4)cc3)cc2)cc1 ; CC(C)(C)c1cc(-c2ccc(N3c4cc(N(c5ccccc5)c5ccccc5)cc5c4B(c4cc(-c6ccccc6)ccc4N5c4ccc(-c5ccccc5)cc4)c4c(-c5ccccc5)nn(-c5ccccc5)c43)cc2)cc(C(C)(C)C)c1]

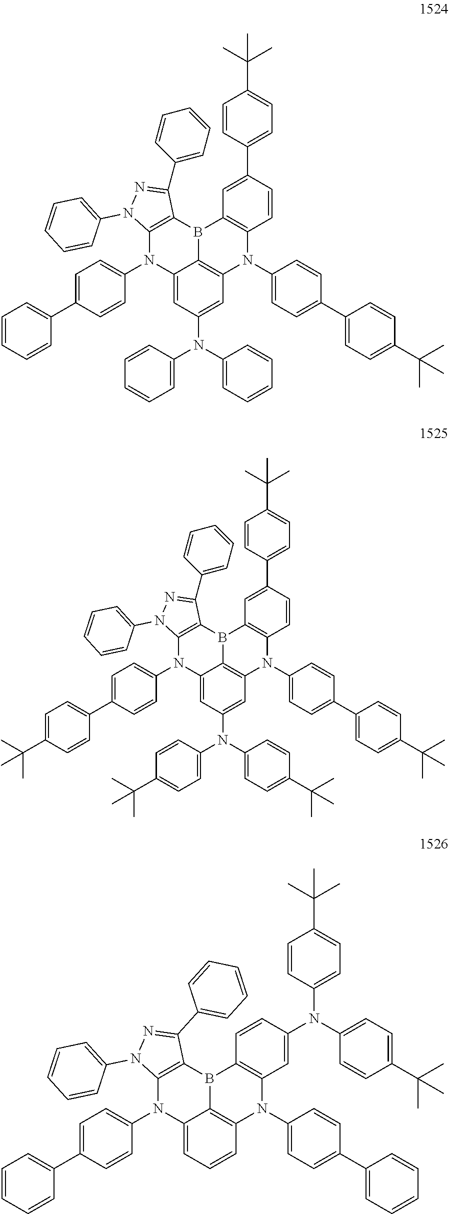

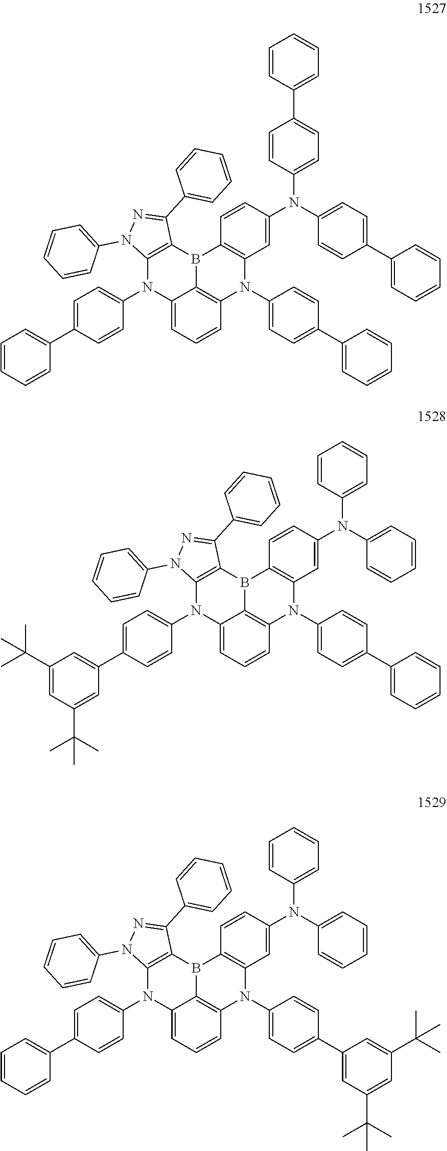 [SMILES: c1ccc(-c2ccc(N(c3ccc(-c4ccccc4)cc3)c3ccc4c(c3)N(c3ccc(-c5ccccc5)cc3)c3cccc5c3B4c3c(-c4ccccc4)nn(-c4ccccc4)c3N5c3ccc(-c4ccccc4)cc3)cc2)cc1 ; CC(C)(C)c1cc(-c2ccc(N3c4cccc5c4B(c4ccc(N(c6ccccc6)c6ccccc6)cc4N5c4ccc(-c5ccccc5)cc4)c4c(-c5ccccc5)nn(-c5ccccc5)c43)cc2)cc(C(C)(C)C)c1 ; CC(C)(C)c1cc(-c2ccc(N3c4cc(N(c5ccccc5)c5ccccc5)ccc4B4c5c3cccc5N(c3ccc(-c5ccccc5)cc3)c3c4c(-c4ccccc4)nn3-c3ccccc3)cc2)cc(C(C)(C)C)c1]

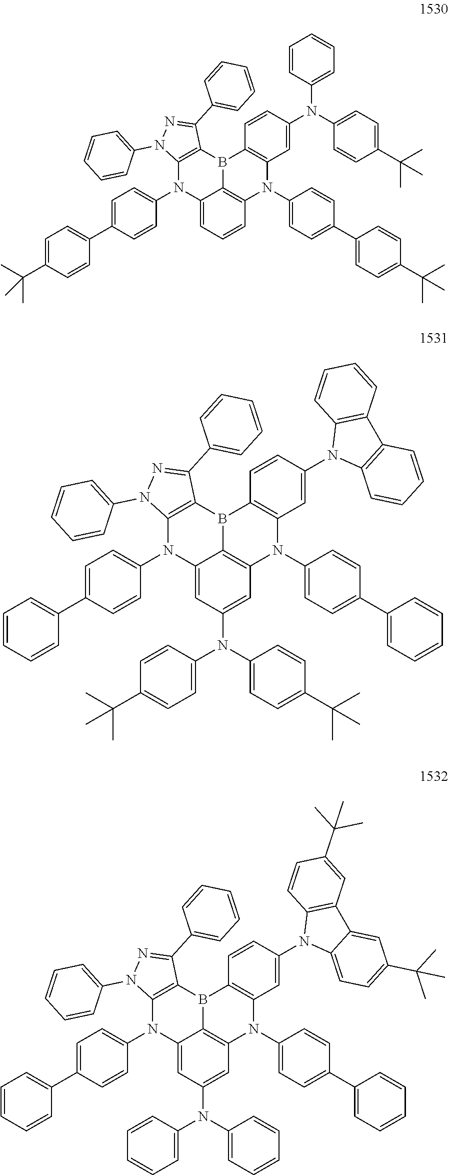 [SMILES: CC(C)(C)c1ccc(-c2ccc(N3c4cc(N(c5ccccc5)c5ccc(C(C)(C)C)cc5)ccc4B4c5c3cccc5N(c3ccc(-c5ccc(C(C)(C)C)cc5)cc3)c3c4c(-c4ccccc4)nn3-c3ccccc3)cc2)cc1 ; CC(C)(C)c1ccc(N(c2ccc(C(C)(C)C)cc2)c2cc3c4c(c2)N(c2ccc(-c5ccccc5)cc2)c2c(c(-c5ccccc5)nn2-c2ccccc2)B4c2ccc(-n4c5ccccc5c5ccccc54)cc2N3c2ccc(-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1c(-c2ccccc2)nn(-c2ccccc2)c1N3c1ccc(-c2ccccc2)cc1]

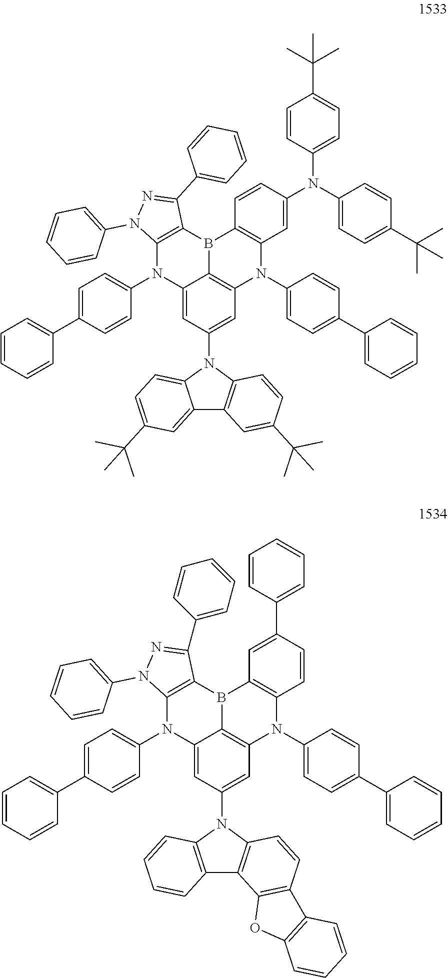 [SMILES: CC(C)(C)c1ccc(N(c2ccc(C(C)(C)C)cc2)c2ccc3c(c2)N(c2ccc(-c4ccccc4)cc2)c2cc(-n4c5ccc(C(C)(C)C)cc5c5cc(C(C)(C)C)ccc54)cc4c2B3c2c(-c3ccccc3)nn(-c3ccccc3)c2N4c2ccc(-c3ccccc3)cc2)cc1 ; c1ccc(-c2ccc(N3c4ccc(-c5ccccc5)cc4B4c5c3cc(-n3c6ccccc6c6c7oc8ccccc8c7ccc63)cc5N(c3ccc(-c5ccccc5)cc3)c3c4c(-c4ccccc4)nn3-c3ccccc3)cc2)cc1]

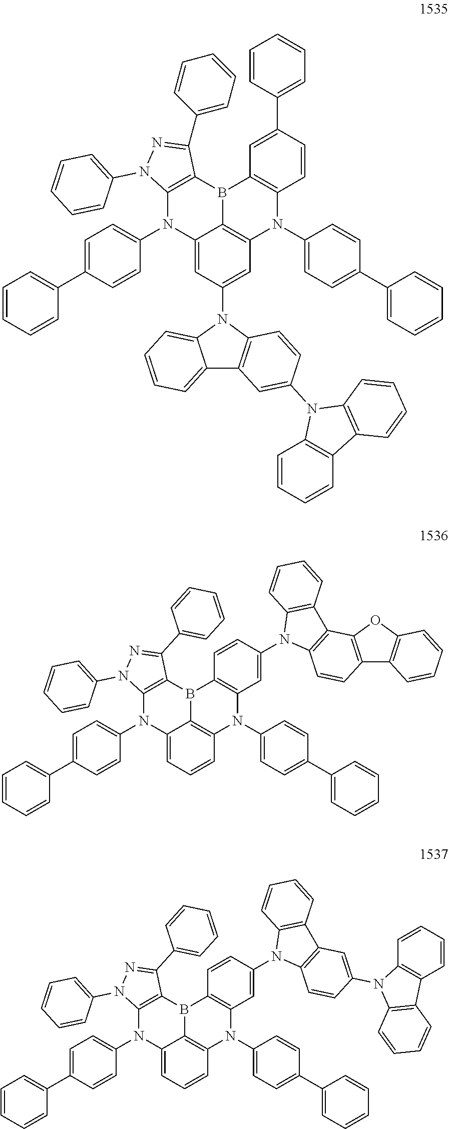 [SMILES: c1ccc(-c2ccc(N3c4ccc(-c5ccccc5)cc4B4c5c3cc(-n3c6ccccc6c6cc(-n7c8ccccc8c8ccccc87)ccc63)cc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6c7oc8ccccc8c7ccc65)ccc4B4c5c3cccc5N(c3ccc(-c5ccccc5)cc3)c3c4c(-c4ccccc4)nn3-c3ccccc3)cc2)cc1 ; c1ccc(-c2ccc(N3c4cc(-n5c6ccccc6c6cc(-n7c8ccccc8c8ccccc87)ccc65)ccc4B4c5c3cccc5N(c3ccc(-c5ccccc5)cc3)c3c4c(-c4ccccc4)nn3-c3ccccc3)cc2)cc1]

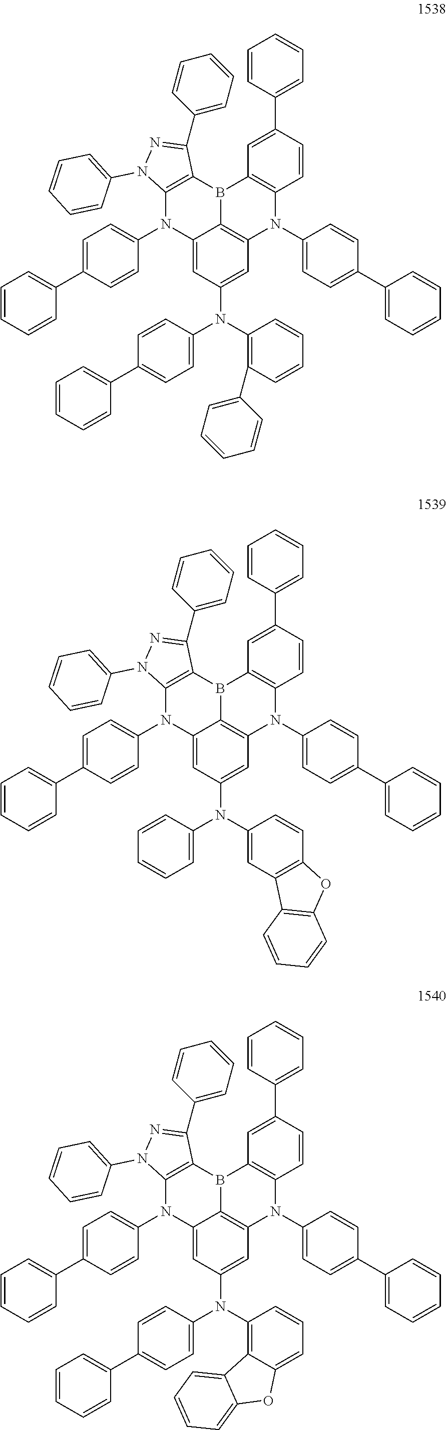 [SMILES: c1ccc(-c2ccc(N(c3cc4c5c(c3)N(c3ccc(-c6ccccc6)cc3)c3c(c(-c6ccccc6)nn3-c3ccccc3)B5c3cc(-c5ccccc5)ccc3N4c3ccc(-c4ccccc4)cc3)c3ccccc3-c3ccccc3)cc2)cc1 ; c1ccc(-c2ccc(N3c4ccc(-c5ccccc5)cc4B4c5c3cc(N(c3ccccc3)c3ccc6oc7ccccc7c6c3)cc5N(c3ccc(-c5ccccc5)cc3)c3c4c(-c4ccccc4)nn3-c3ccccc3)cc2)cc1 ; c1ccc(-c2ccc(N3c4ccc(-c5ccccc5)cc4B4c5c3cc(N(c3ccc(-c6ccccc6)cc3)c3cccc6oc7ccccc7c36)cc5N(c3ccc(-c5ccccc5)cc3)c3c4c(-c4ccccc4)nn3-c3ccccc3)cc2)cc1]

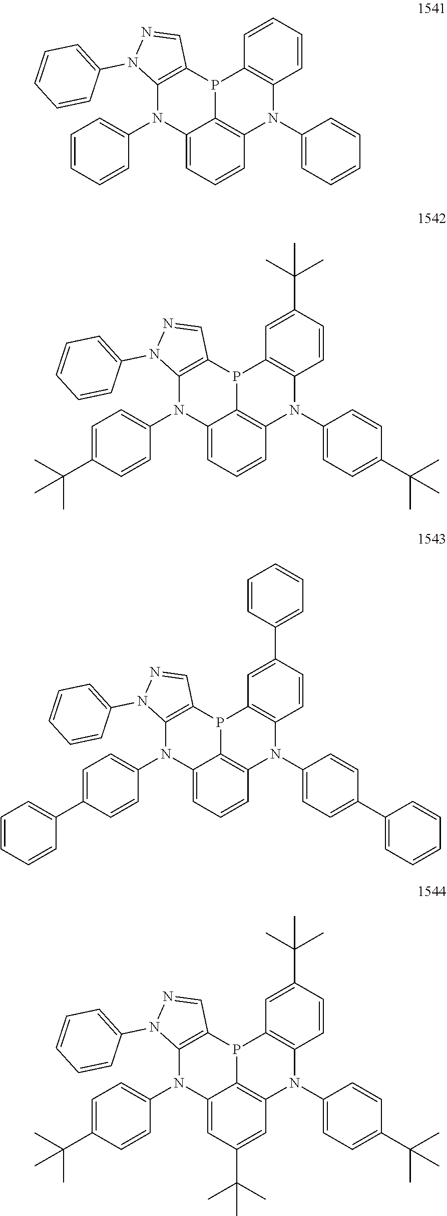

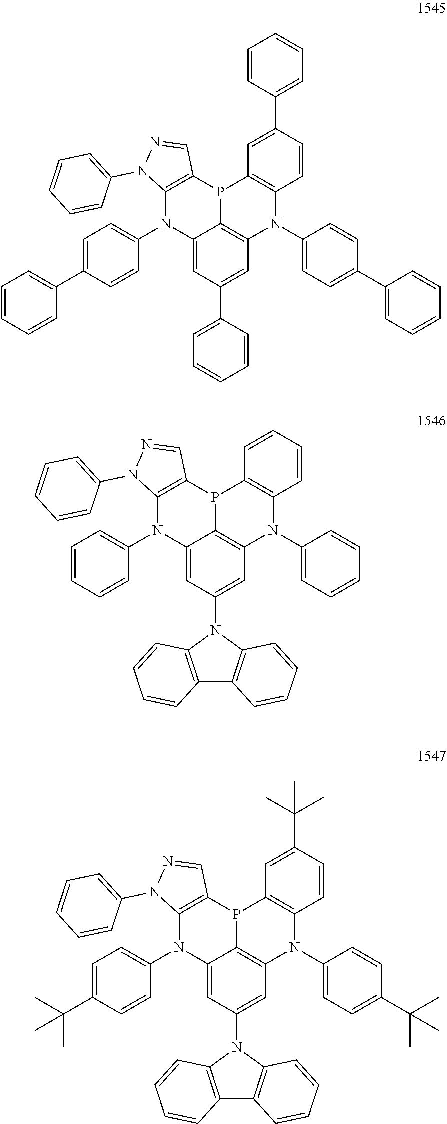 [SMILES: c1ccc(-c2ccc(N3c4ccc(-c5ccccc5)cc4P4c5cnn(-c6ccccc6)c5N(c5ccc(-c6ccccc6)cc5)c5cc(-c6ccccc6)cc3c54)cc2)cc1 ; c1ccc(N2c3ccccc3P3c4cnn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3P3c4cnn(-c5ccccc5)c4N(c4ccc(C(C)(C)C)cc4)c4cc(-n5c6ccccc6c6ccccc65)cc2c43)cc1]

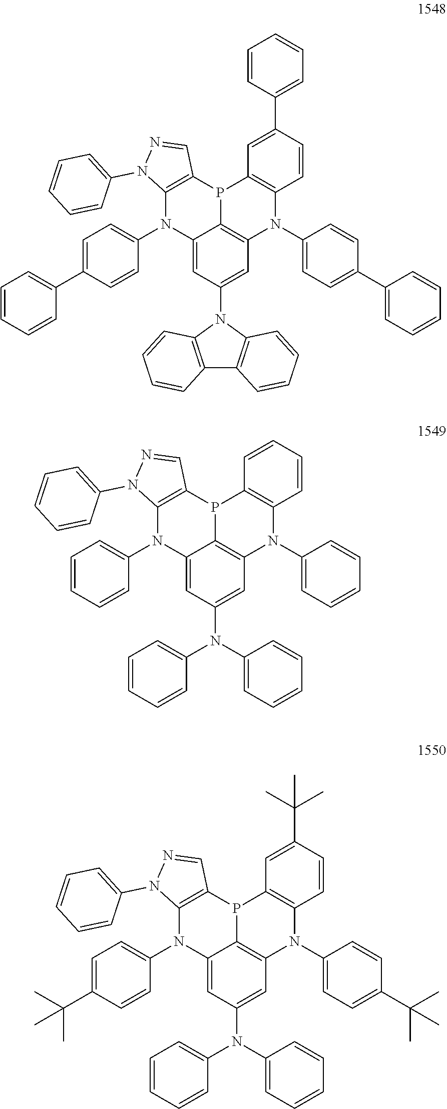 [SMILES: c1ccc(-c2ccc(N3c4ccc(-c5ccccc5)cc4P4c5cnn(-c6ccccc6)c5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2c(cnn2-c2ccccc2)P4c2ccccc2N3c2ccccc2)cc1 ; CC(C)(C)c1ccc(N2c3ccc(C(C)(C)C)cc3P3c4cnn(-c5ccccc5)c4N(c4ccc(C(C)(C)C)cc4)c4cc(N(c5ccccc5)c5ccccc5)cc2c43)cc1]

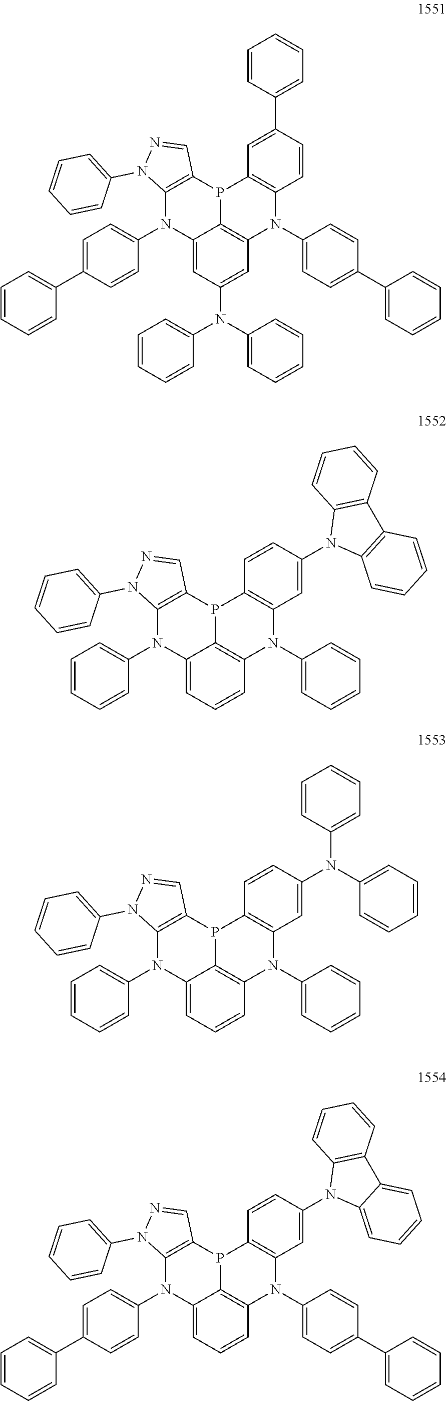 [SMILES: c1ccc(-c2ccc(N3c4ccc(-c5ccccc5)cc4P4c5cnn(-c6ccccc6)c5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3P3c4cnn(-c5ccccc5)c4N(c4ccccc4)c4cccc2c43)cc1 ; c1ccc(N(c2ccccc2)c2ccc3c(c2)N(c2ccccc2)c2cccc4c2P3c2cnn(-c3ccccc3)c2N4c2ccccc2)cc1 ; c1ccc(-c2ccc(N3c4cc(-n5c6ccccc6c6ccccc65)ccc4P4c5cnn(-c6ccccc6)c5N(c5ccc(-c6ccccc6)cc5)c5cccc3c54)cc2)cc1]

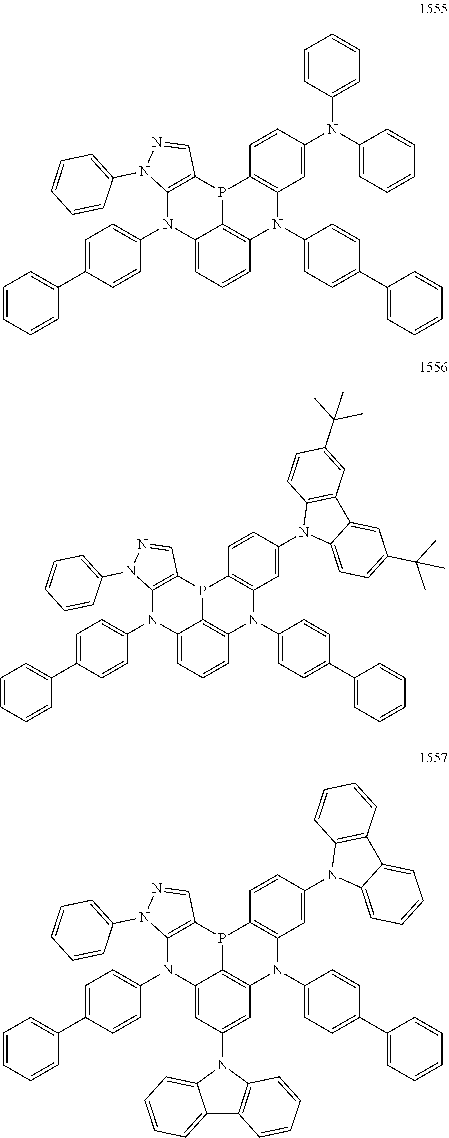 [SMILES: c1ccc(-c2ccc(N3c4cc(N(c5ccccc5)c5ccccc5)ccc4P4c5cnn(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1cnn(-c2ccccc2)c1N3c1ccc(-c2ccccc2)cc1 ; c1ccc(-c2ccc(N3c4cc(-n5c6ccccc6c6ccccc65)ccc4P4c5cnn(-c6ccccc6)c5N(c5ccc(-c6ccccc6)cc5)c5cc(-n6c7ccccc7c7ccccc76)cc3c54)cc2)cc1]

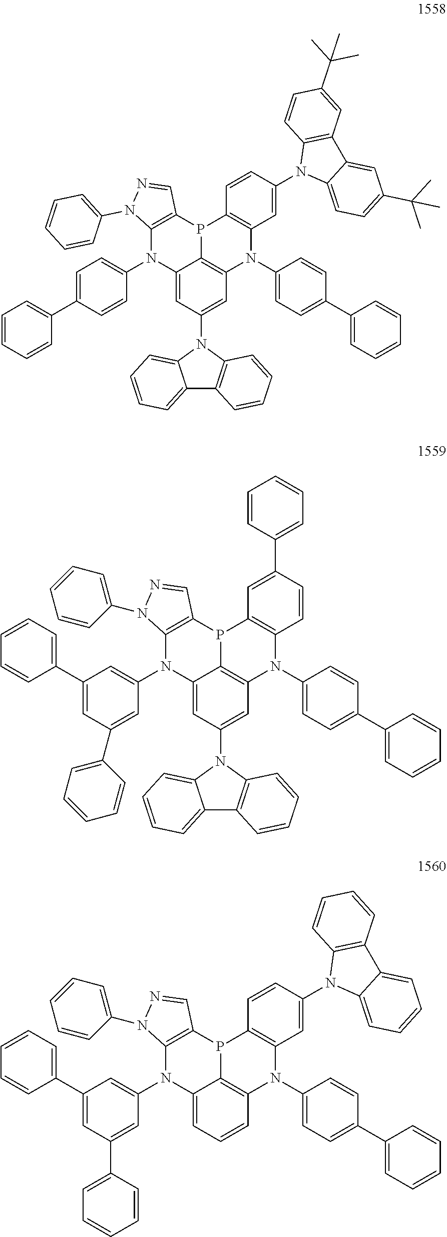 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1cnn(-c2ccccc2)c1N3c1ccc(-c2ccccc2)cc1 ; c1ccc(-c2ccc(N3c4ccc(-c5ccccc5)cc4P4c5cnn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4P4c5cnn(-c6ccccc6)c5N(c5cc(-c6ccccc6)cc(-c6ccccc6)c5)c5cccc3c54)cc2)cc1]

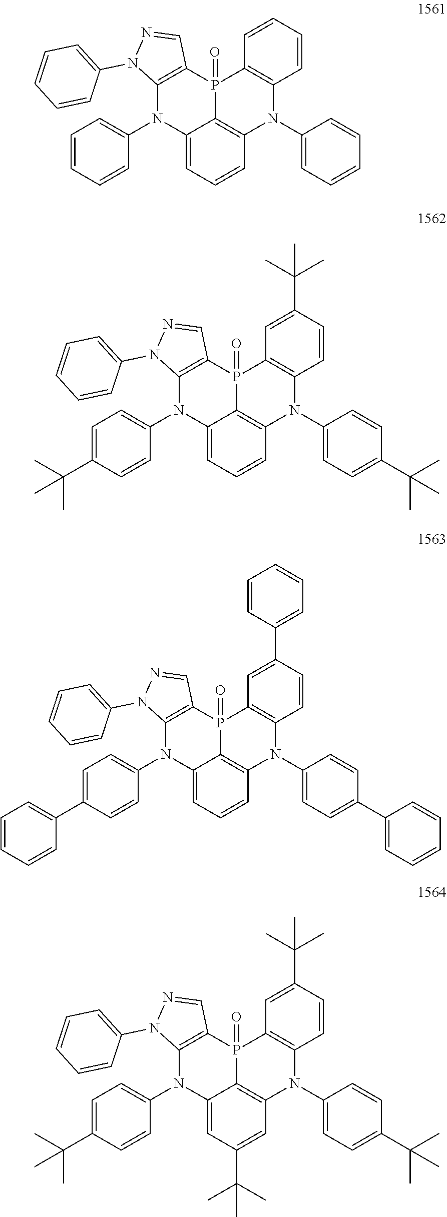

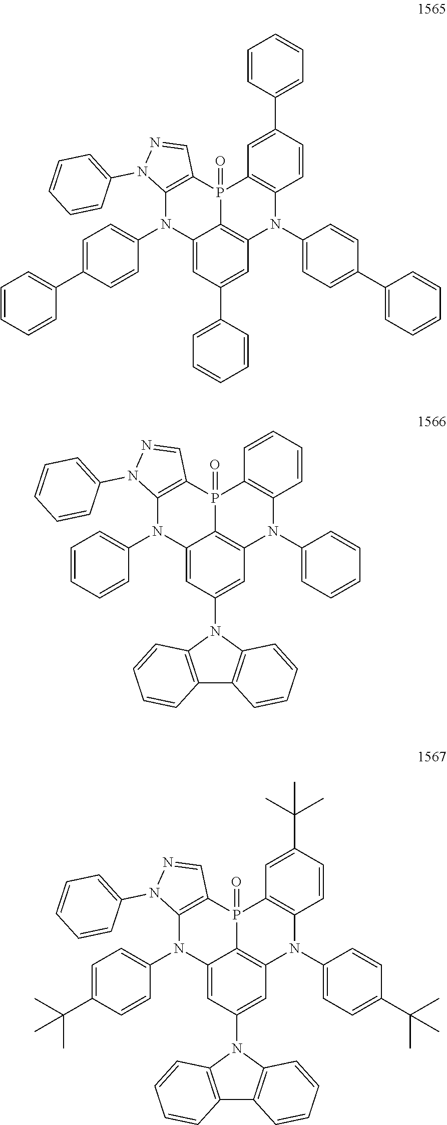 [SMILES: O=P12c3cc(-c4ccccc4)ccc3N(c3ccc(-c4ccccc4)cc3)c3cc(-c4ccccc4)cc(c31)N(c1ccc(-c3ccccc3)cc1)c1c2cnn1-c1ccccc1 ; O=P12c3ccccc3N(c3ccccc3)c3cc(-n4c5ccccc5c5ccccc54)cc(c31)N(c1ccccc1)c1c2cnn1-c1ccccc1 ; CC(C)(C)c1ccc(N2c3ccc(C(C)(C)C)cc3P3(=O)c4cnn(-c5ccccc5)c4N(c4ccc(C(C)(C)C)cc4)c4cc(-n5c6ccccc6c6ccccc65)cc2c43)cc1]

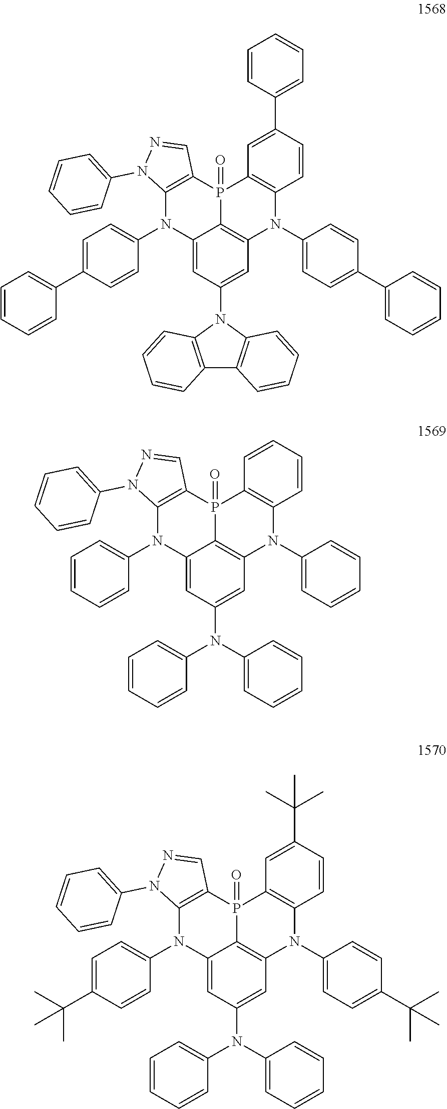 [SMILES: O=P12c3cc(-c4ccccc4)ccc3N(c3ccc(-c4ccccc4)cc3)c3cc(-n4c5ccccc5c5ccccc54)cc(c31)N(c1ccc(-c3ccccc3)cc1)c1c2cnn1-c1ccccc1 ; O=P12c3ccccc3N(c3ccccc3)c3cc(N(c4ccccc4)c4ccccc4)cc(c31)N(c1ccccc1)c1c2cnn1-c1ccccc1 ; CC(C)(C)c1ccc(N2c3ccc(C(C)(C)C)cc3P3(=O)c4cnn(-c5ccccc5)c4N(c4ccc(C(C)(C)C)cc4)c4cc(N(c5ccccc5)c5ccccc5)cc2c43)cc1]

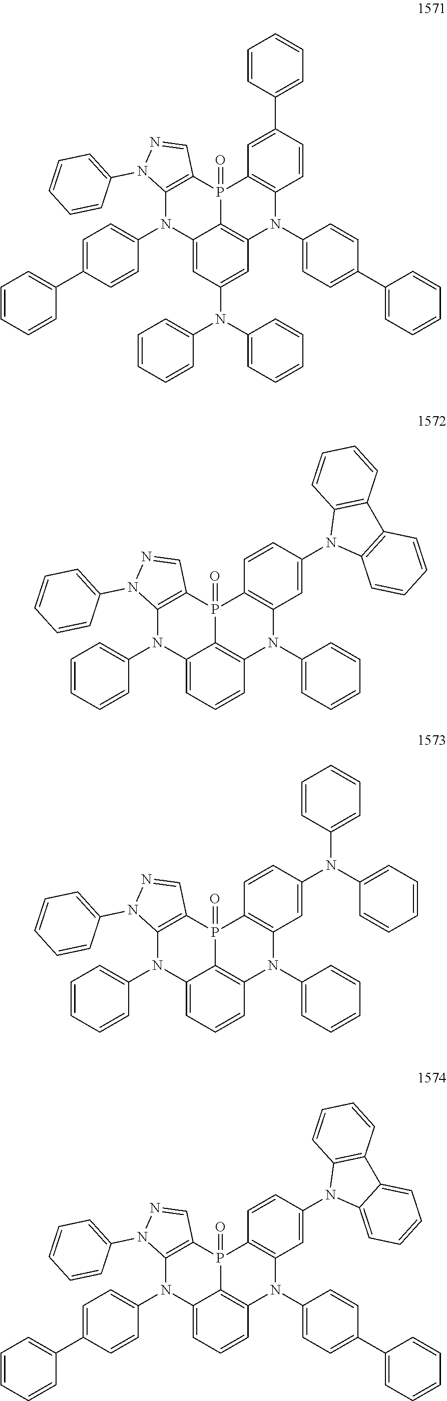 [SMILES: O=P12c3cc(-c4ccccc4)ccc3N(c3ccc(-c4ccccc4)cc3)c3cc(N(c4ccccc4)c4ccccc4)cc(c31)N(c1ccc(-c3ccccc3)cc1)c1c2cnn1-c1ccccc1 ; O=P12c3ccc(-n4c5ccccc5c5ccccc54)cc3N(c3ccccc3)c3cccc(c31)N(c1ccccc1)c1c2cnn1-c1ccccc1 ; O=P12c3ccc(N(c4ccccc4)c4ccccc4)cc3N(c3ccccc3)c3cccc(c31)N(c1ccccc1)c1c2cnn1-c1ccccc1 ; O=P12c3ccc(-n4c5ccccc5c5ccccc54)cc3N(c3ccc(-c4ccccc4)cc3)c3cccc(c31)N(c1ccc(-c3ccccc3)cc1)c1c2cnn1-c1ccccc1]

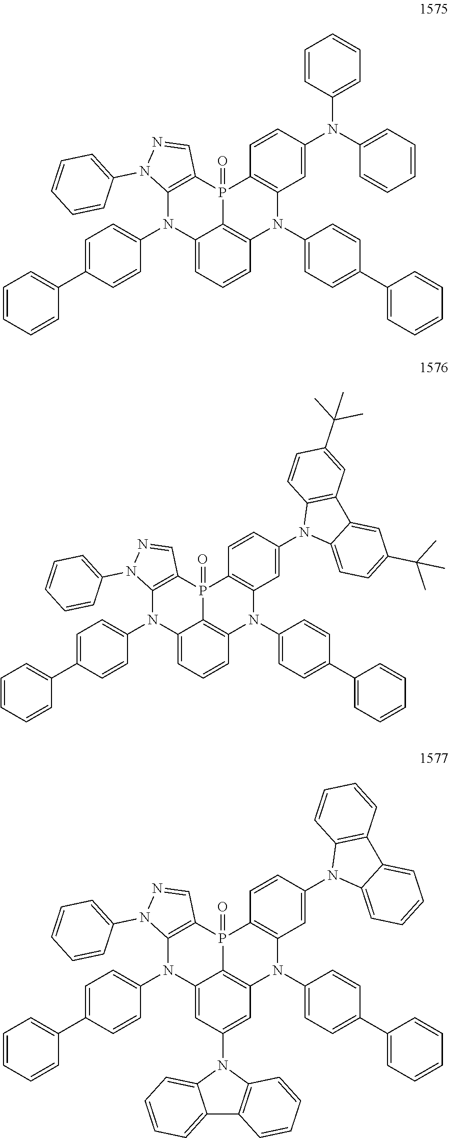 [SMILES: O=P12c3ccc(N(c4ccccc4)c4ccccc4)cc3N(c3ccc(-c4ccccc4)cc3)c3cccc(c31)N(c1ccc(-c3ccccc3)cc1)c1c2cnn1-c1ccccc1 ; CC(C)(C)c1ccc2c(c1)c1cc(C(C)(C)C)ccc1n2-c1ccc2c(c1)N(c1ccc(-c3ccccc3)cc1)c1cccc3c1P2(=O)c1cnn(-c2ccccc2)c1N3c1ccc(-c2ccccc2)cc1 ; O=P12c3ccc(-n4c5ccccc5c5ccccc54)cc3N(c3ccc(-c4ccccc4)cc3)c3cc(-n4c5ccccc5c5ccccc54)cc(c31)N(c1ccc(-c3ccccc3)cc1)c1c2cnn1-c1ccccc1]

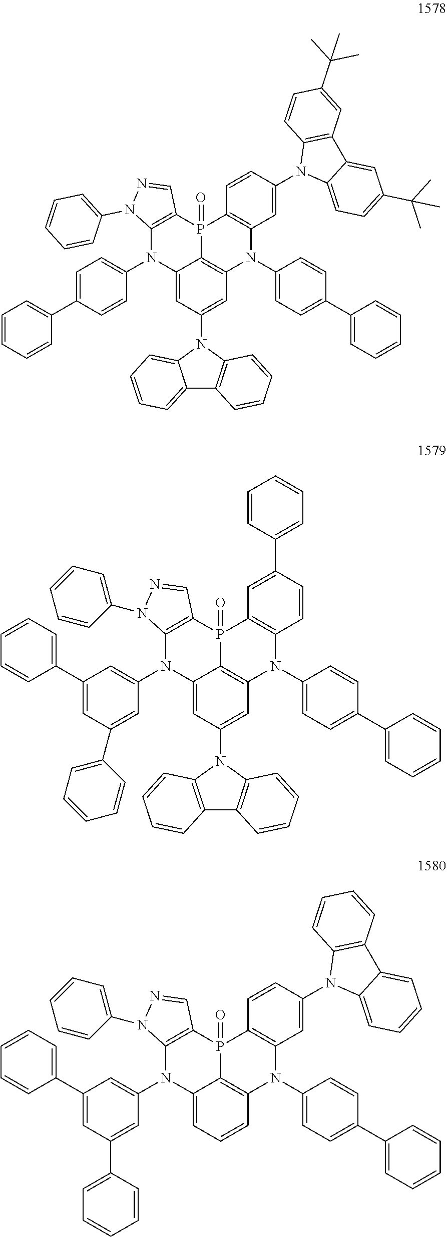 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2(=O)c1cnn(-c2ccccc2)c1N3c1ccc(-c2ccccc2)cc1 ; O=P12c3cc(-c4ccccc4)ccc3N(c3ccc(-c4ccccc4)cc3)c3cc(-n4c5ccccc5c5ccccc54)cc(c31)N(c1cc(-c3ccccc3)cc(-c3ccccc3)c1)c1c2cnn1-c1ccccc1 ; O=P12c3ccc(-n4c5ccccc5c5ccccc54)cc3N(c3ccc(-c4ccccc4)cc3)c3cccc(c31)N(c1cc(-c3ccccc3)cc(-c3ccccc3)c1)c1c2cnn1-c1ccccc1]

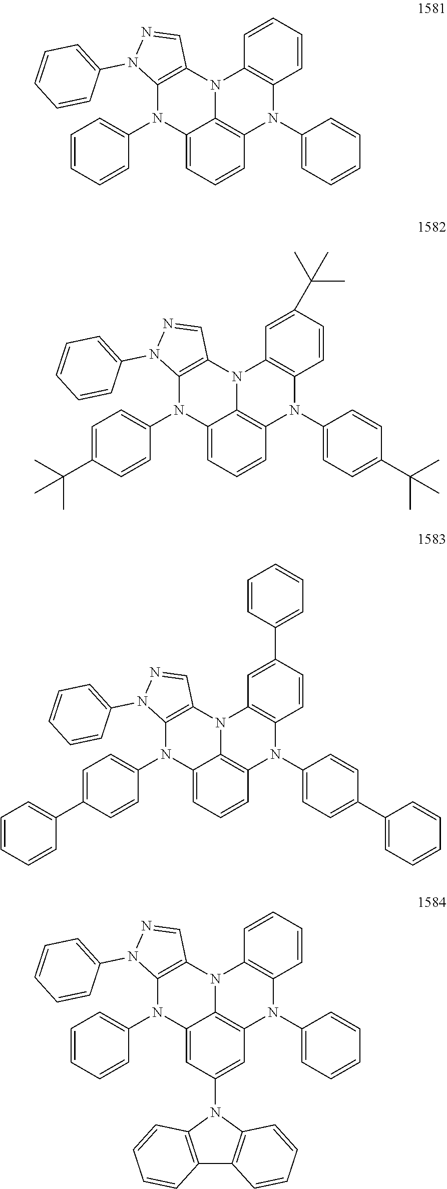 [SMILES: c1ccc(N2c3ccccc3N3c4cnn(-c5ccccc5)c4N(c4ccccc4)c4cccc2c43)cc1 ; CC(C)(C)c1ccc(N2c3ccc(C(C)(C)C)cc3N3c4cnn(-c5ccccc5)c4N(c4ccc(C(C)(C)C)cc4)c4cccc2c43)cc1 ; c1ccc(-c2ccc(N3c4ccc(-c5ccccc5)cc4N4c5cnn(-c6ccccc6)c5N(c5ccc(-c6ccccc6)cc5)c5cccc3c54)cc2)cc1 ; c1ccc(N2c3ccccc3N3c4cnn(-c5ccccc5)c4N(c4ccccc4)c4cc(-n5c6ccccc6c6ccccc65)cc2c43)cc1]

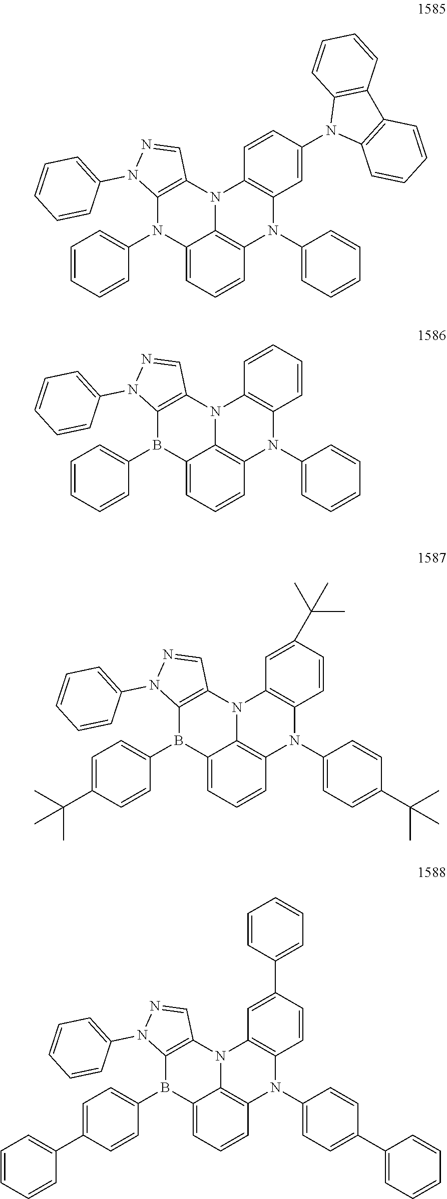 [SMILES: c1ccc(N2c3cc(-n4c5ccccc5c5ccccc54)ccc3N3c4cnn(-c5ccccc5)c4N(c4ccccc4)c4cccc2c43)cc1 ; c1ccc(B2c3cccc4c3N(c3ccccc3N4c3ccccc3)c3cnn(-c4ccccc4)c32)cc1 ; CC(C)(C)c1ccc(B2c3cccc4c3N(c3cc(C(C)(C)C)ccc3N4c3ccc(C(C)(C)C)cc3)c3cnn(-c4ccccc4)c32)cc1 ; c1ccc(-c2ccc(B3c4cccc5c4N(c4cc(-c6ccccc6)ccc4N5c4ccc(-c5ccccc5)cc4)c4cnn(-c5ccccc5)c43)cc2)cc1]

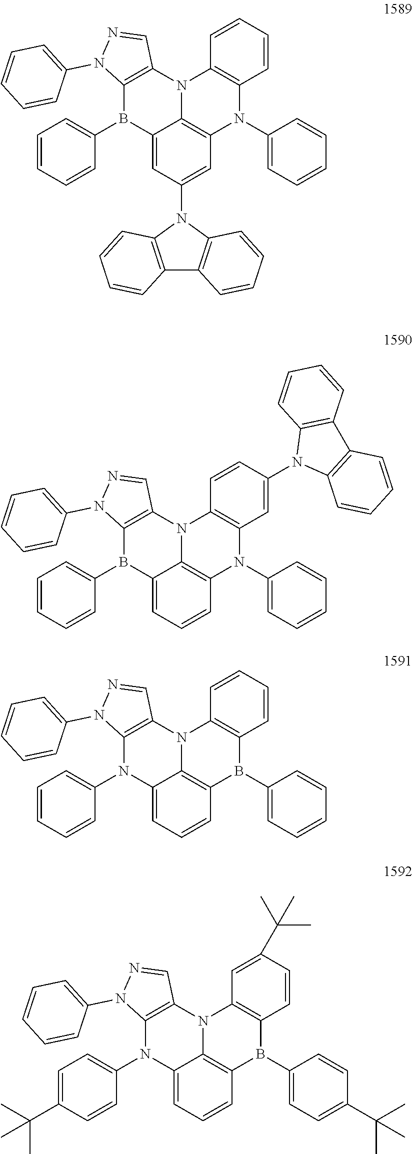 [SMILES: c1ccc(B2c3cc(-n4c5ccccc5c5ccccc54)cc4c3N(c3ccccc3N4c3ccccc3)c3cnn(-c4ccccc4)c32)cc1 ; c1ccc(B2c3cccc4c3N(c3ccc(-n5c6ccccc6c6ccccc65)cc3N4c3ccccc3)c3cnn(-c4ccccc4)c32)cc1 ; c1ccc(B2c3ccccc3N3c4cnn(-c5ccccc5)c4N(c4ccccc4)c4cccc2c43)cc1 ; CC(C)(C)c1ccc(B2c3ccc(C(C)(C)C)cc3N3c4cnn(-c5ccccc5)c4N(c4ccc(C(C)(C)C)cc4)c4cccc2c43)cc1]

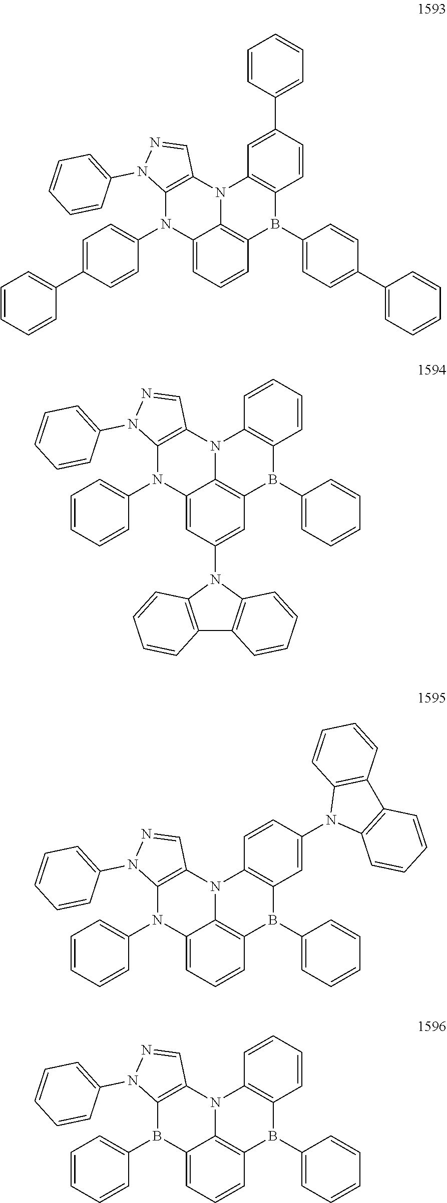 [SMILES: c1ccc(-c2ccc(B3c4ccc(-c5ccccc5)cc4N4c5cnn(-c6ccccc6)c5N(c5ccc(-c6ccccc6)cc5)c5cccc3c54)cc2)cc1 ; c1ccc(B2c3ccccc3N3c4cnn(-c5ccccc5)c4N(c4ccccc4)c4cc(-n5c6ccccc6c6ccccc65)cc2c43)cc1 ; c1ccc(B2c3cc(-n4c5ccccc5c5ccccc54)ccc3N3c4cnn(-c5ccccc5)c4N(c4ccccc4)c4cccc2c43)cc1 ; c1ccc(B2c3ccccc3N3c4cnn(-c5ccccc5)c4B(c4ccccc4)c4cccc2c43)cc1]

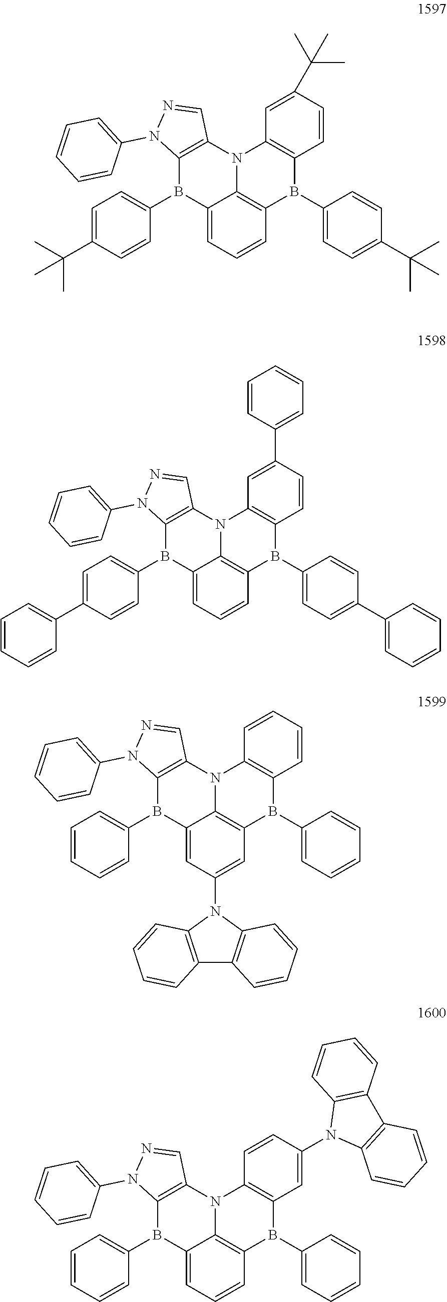 [SMILES: CC(C)(C)c1ccc(B2c3ccc(C(C)(C)C)cc3N3c4cnn(-c5ccccc5)c4B(c4ccc(C(C)(C)C)cc4)c4cccc2c43)cc1 ; c1ccc(-c2ccc(B3c4ccc(-c5ccccc5)cc4N4c5cnn(-c6ccccc6)c5B(c5ccc(-c6ccccc6)cc5)c5cccc3c54)cc2)cc1 ; c1ccc(B2c3ccccc3N3c4cnn(-c5ccccc5)c4B(c4ccccc4)c4cc(-n5c6ccccc6c6ccccc65)cc2c43)cc1 ; c1ccc(B2c3cc(-n4c5ccccc5c5ccccc54)ccc3N3c4cnn(-c5ccccc5)c4B(c4ccccc4)c4cccc2c43)cc1]

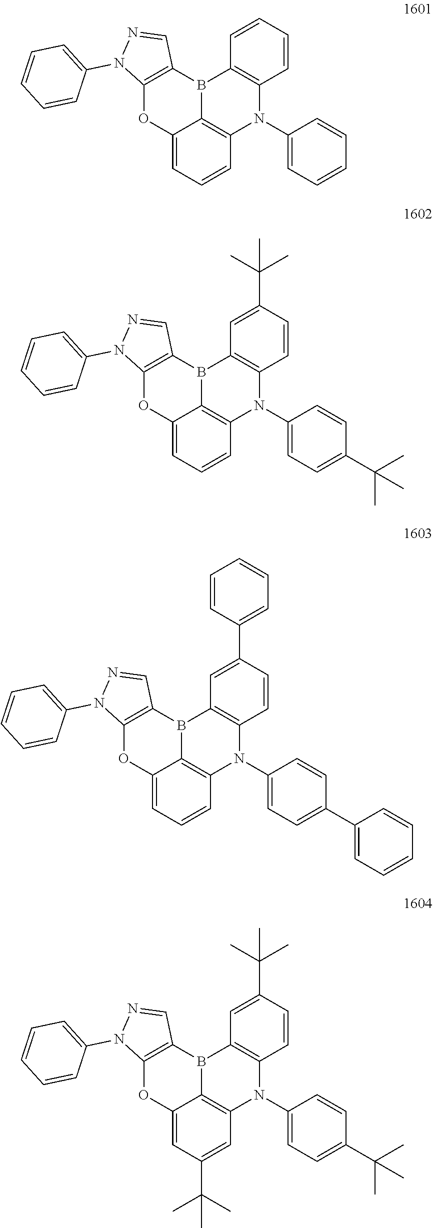 [SMILES: c1ccc(N2c3ccccc3B3c4cnn(-c5ccccc5)c4Oc4cccc2c43)cc1 ; CC(C)(C)c1ccc(N2c3ccc(C(C)(C)C)cc3B3c4cnn(-c5ccccc5)c4Oc4cccc2c43)cc1 ; c1ccc(-c2ccc(N3c4ccc(-c5ccccc5)cc4B4c5cnn(-c6ccccc6)c5Oc5cccc3c54)cc2)cc1 ; CC(C)(C)c1ccc(N2c3ccc(C(C)(C)C)cc3B3c4cnn(-c5ccccc5)c4Oc4cc(C(C)(C)C)cc2c43)cc1]

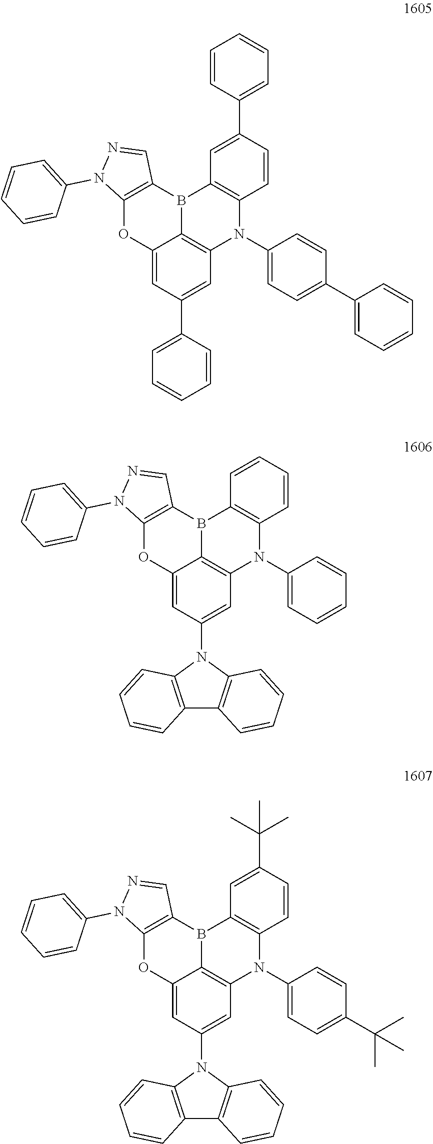 [SMILES: c1ccc(-c2ccc(N3c4ccc(-c5ccccc5)cc4B4c5cnn(-c6ccccc6)c5Oc5cc(-c6ccccc6)cc3c54)cc2)cc1 ; c1ccc(N2c3ccccc3B3c4cnn(-c5ccccc5)c4Oc4cc(-n5c6ccccc6c6ccccc65)cc2c43)cc1 ; CC(C)(C)c1ccc(N2c3ccc(C(C)(C)C)cc3B3c4cnn(-c5ccccc5)c4Oc4cc(-n5c6ccccc6c6ccccc65)cc2c43)cc1]

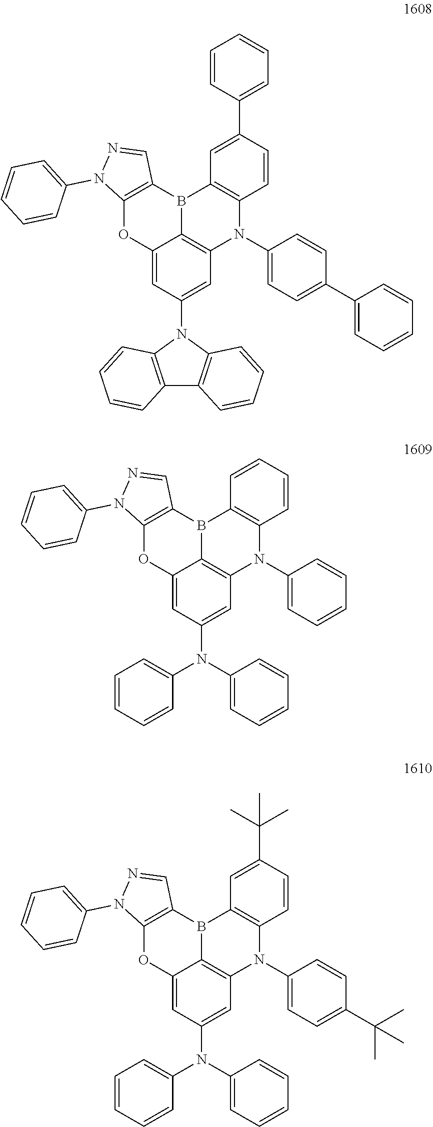 [SMILES: c1ccc(-c2ccc(N3c4ccc(-c5ccccc5)cc4B4c5cnn(-c6ccccc6)c5Oc5cc(-n6c7ccccc7c7ccccc76)cc3c54)cc2)cc1 ; c1ccc(N(c2ccccc2)c2cc3c4c(c2)N(c2ccccc2)c2ccccc2B4c2cnn(-c4ccccc4)c2O3)cc1 ; CC(C)(C)c1ccc(N2c3ccc(C(C)(C)C)cc3B3c4cnn(-c5ccccc5)c4Oc4cc(N(c5ccccc5)c5ccccc5)cc2c43)cc1]

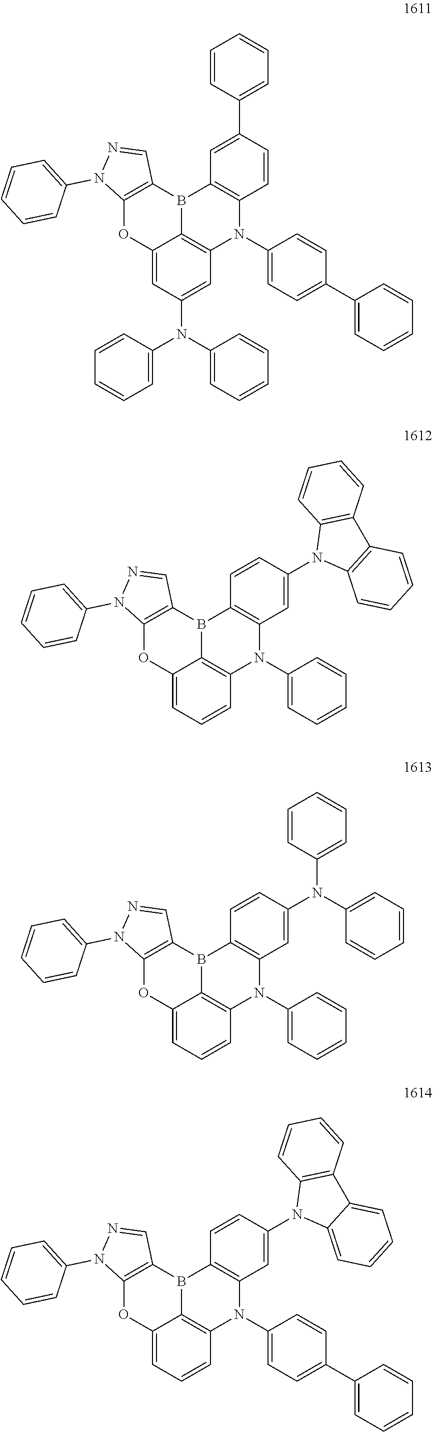 [SMILES: c1ccc(-c2ccc(N3c4ccc(-c5ccccc5)cc4B4c5cnn(-c6ccccc6)c5Oc5cc(N(c6ccccc6)c6ccccc6)cc3c54)cc2)cc1 ; c1ccc(N2c3cc(-n4c5ccccc5c5ccccc54)ccc3B3c4cnn(-c5ccccc5)c4Oc4cccc2c43)cc1 ; c1ccc(N(c2ccccc2)c2ccc3c(c2)N(c2ccccc2)c2cccc4c2B3c2cnn(-c3ccccc3)c2O4)cc1 ; c1ccc(-c2ccc(N3c4cc(-n5c6ccccc6c6ccccc65)ccc4B4c5cnn(-c6ccccc6)c5Oc5cccc3c54)cc2)cc1]

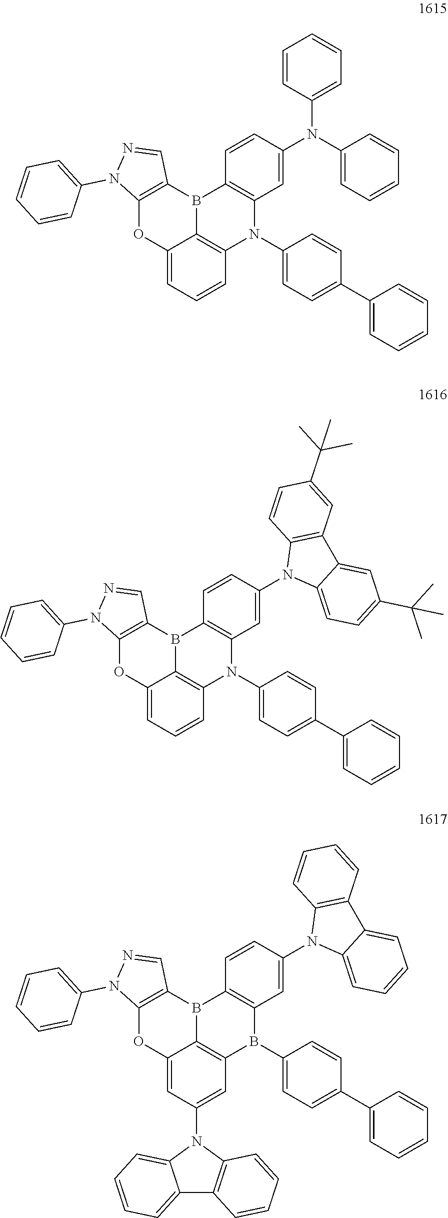 [SMILES: c1ccc(-c2ccc(N3c4cc(N(c5ccccc5)c5ccccc5)ccc4B4c5cnn(-c6ccccc6)c5Oc5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1cnn(-c2ccccc2)c1O3 ; c1ccc(-c2ccc(B3c4cc(-n5c6ccccc6c6ccccc65)ccc4B4c5cnn(-c6ccccc6)c5Oc5cc(-n6c7ccccc7c7ccccc76)cc3c54)cc2)cc1]

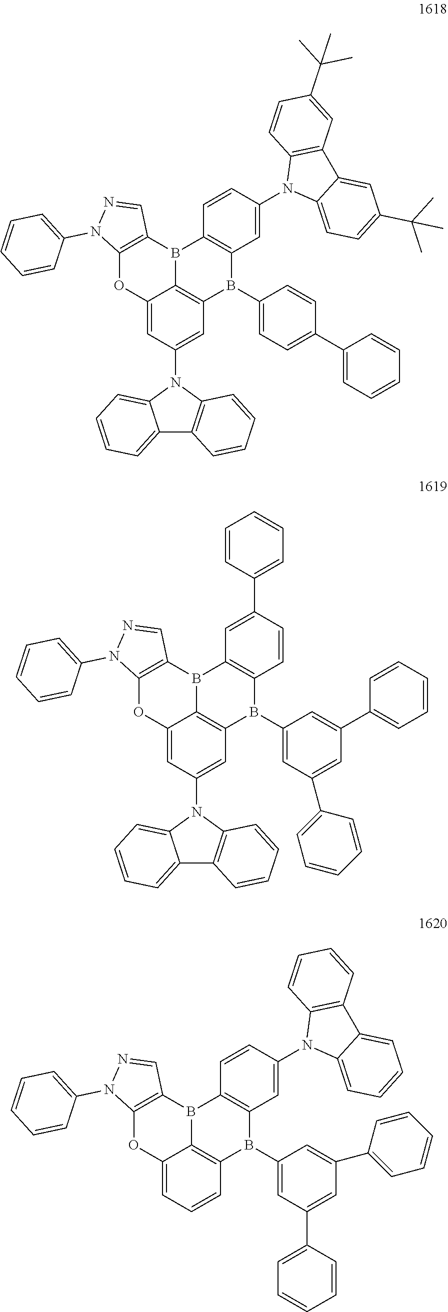 [SMILES: CC(C)(C)c1ccc2c(c1)c1cc(C(C)(C)C)ccc1n2-c1ccc2c(c1)B(c1ccc(-c3ccccc3)cc1)c1cc(-n3c4ccccc4c4ccccc43)cc3c1B2c1cnn(-c2ccccc2)c1O3 ; c1ccc(-c2cc(B3c4ccc(-c5ccccc5)cc4B4c5cnn(-c6ccccc6)c5Oc5cc(-n6c7ccccc7c7ccccc76)cc3c54)cc(-c3ccccc3)c2)cc1 ; c1ccc(-c2cc(B3c4cc(-n5c6ccccc6c6ccccc65)ccc4B4c5cnn(-c6ccccc6)c5Oc5cccc3c54)cc(-c3ccccc3)c2)cc1]

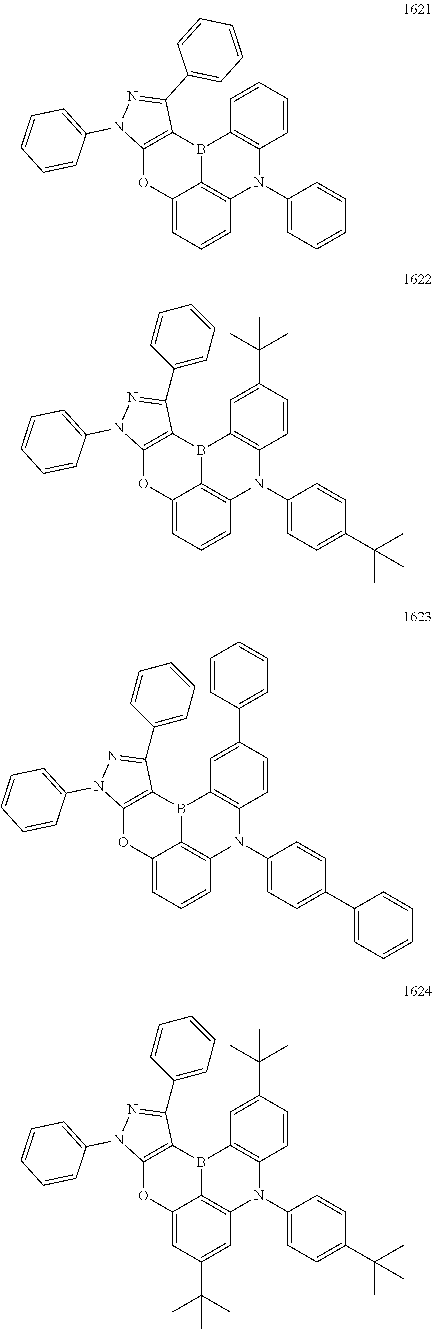 [SMILES: c1ccc(-c2nn(-c3ccccc3)c3c2B2c4ccccc4N(c4ccccc4)c4cccc(c42)O3)cc1 ; CC(C)(C)c1ccc(N2c3ccc(C(C)(C)C)cc3B3c4c(cccc42)Oc2c3c(-c3ccccc3)nn2-c2ccccc2)cc1 ; c1ccc(-c2ccc(N3c4ccc(-c5ccccc5)cc4B4c5c(cccc53)Oc3c4c(-c4ccccc4)nn3-c3ccccc3)cc2)cc1 ; CC(C)(C)c1ccc(N2c3ccc(C(C)(C)C)cc3B3c4c(cc(C(C)(C)C)cc42)Oc2c3c(-c3ccccc3)nn2-c2ccccc2)cc1]

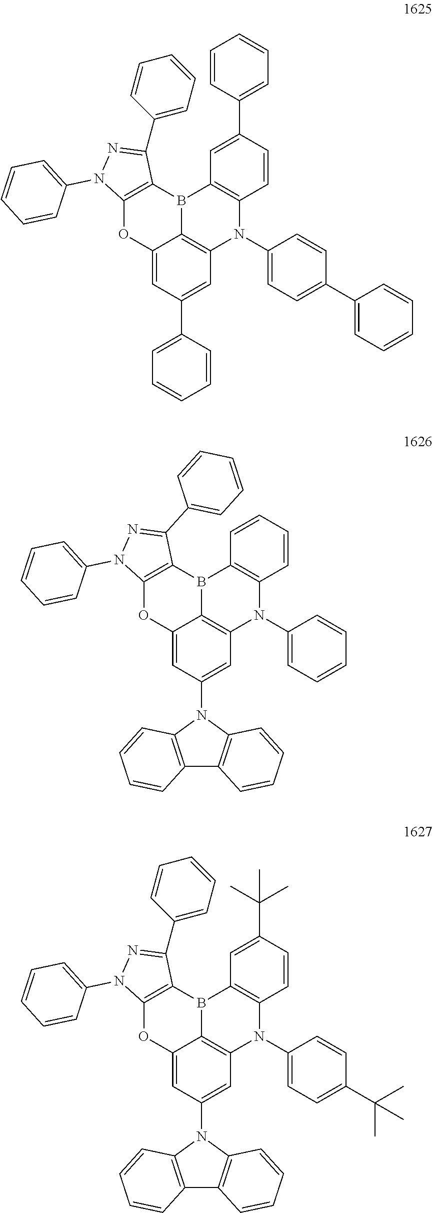 [SMILES: c1ccc(-c2ccc(N3c4ccc(-c5ccccc5)cc4B4c5c(cc(-c6ccccc6)cc53)Oc3c4c(-c4ccccc4)nn3-c3ccccc3)cc2)cc1 ; c1ccc(-c2nn(-c3ccccc3)c3c2B2c4ccccc4N(c4ccccc4)c4cc(-n5c6ccccc6c6ccccc65)cc(c42)O3)cc1 ; CC(C)(C)c1ccc(N2c3ccc(C(C)(C)C)cc3B3c4c(cc(-n5c6ccccc6c6ccccc65)cc42)Oc2c3c(-c3ccccc3)nn2-c2ccccc2)cc1]

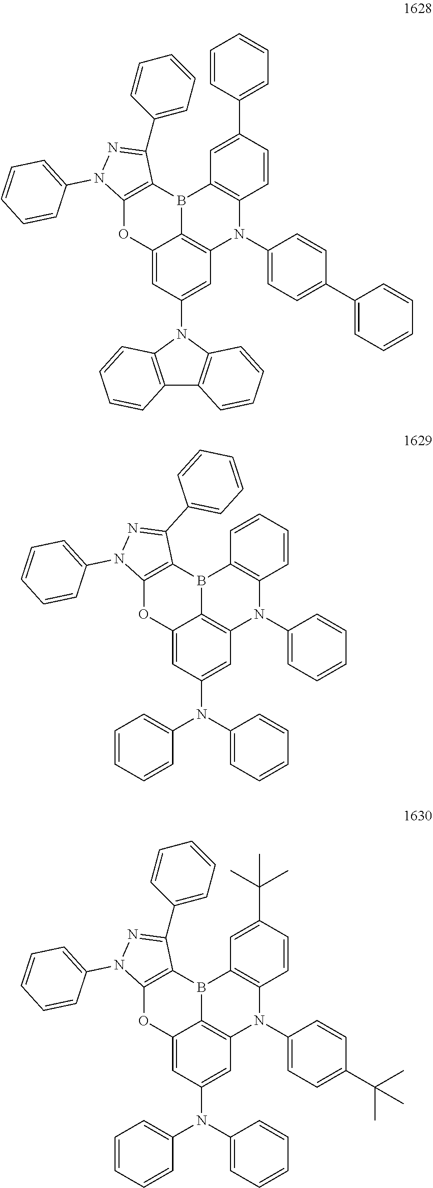

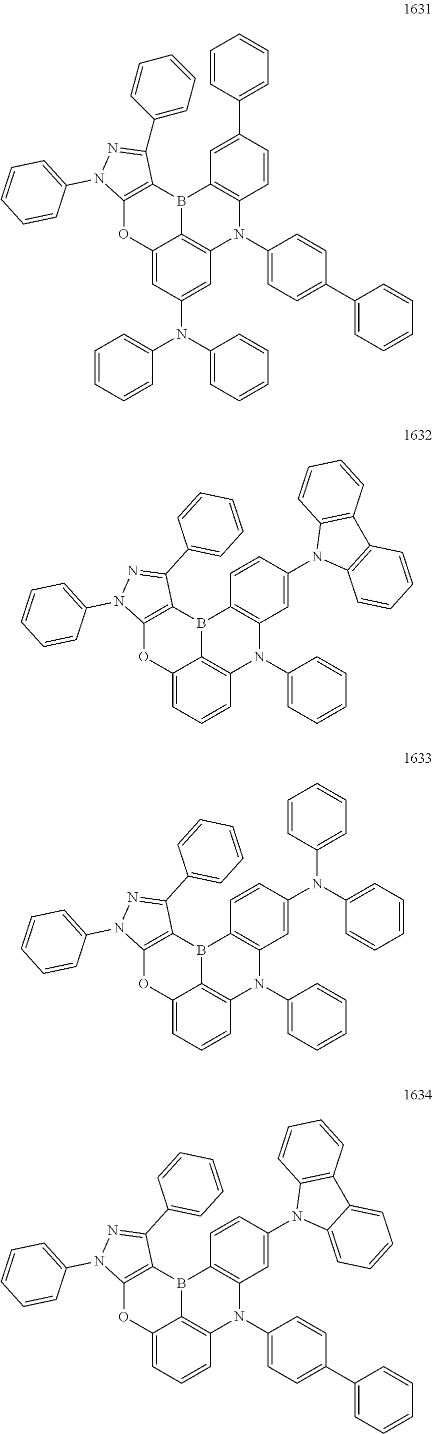 [SMILES: c1ccc(-c2ccc(N3c4ccc(-c5ccccc5)cc4B4c5c(cc(N(c6ccccc6)c6ccccc6)cc53)Oc3c4c(-c4ccccc4)nn3-c3ccccc3)cc2)cc1 ; c1ccc(-c2nn(-c3ccccc3)c3c2B2c4ccc(-n5c6ccccc6c6ccccc65)cc4N(c4ccccc4)c4cccc(c42)O3)cc1 ; c1ccc(-c2nn(-c3ccccc3)c3c2B2c4ccc(N(c5ccccc5)c5ccccc5)cc4N(c4ccccc4)c4cccc(c42)O3)cc1 ; c1ccc(-c2ccc(N3c4cc(-n5c6ccccc6c6ccccc65)ccc4B4c5c(cccc53)Oc3c4c(-c4ccccc4)nn3-c3ccccc3)cc2)cc1]

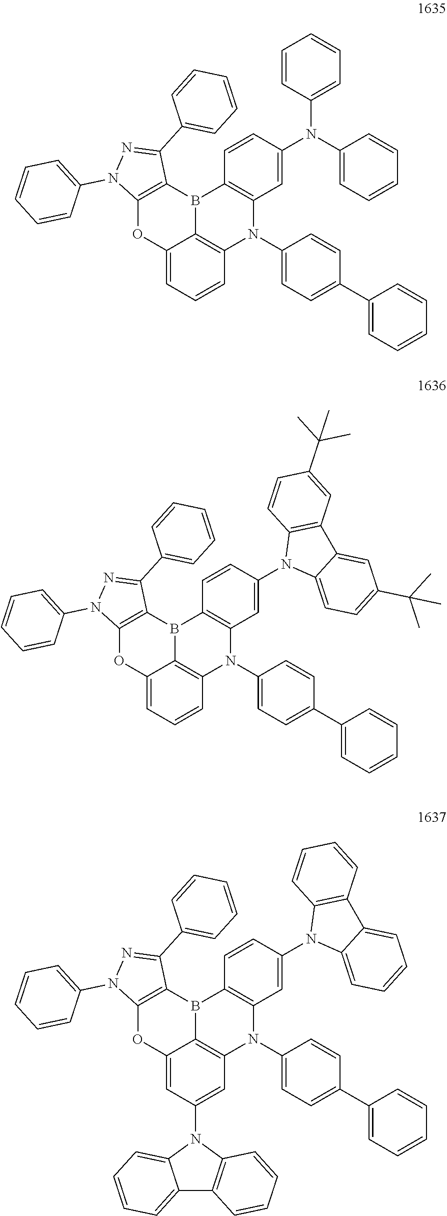 [SMILES: c1ccc(-c2ccc(N3c4cc(N(c5ccccc5)c5ccccc5)ccc4B4c5c(cccc53)Oc3c4c(-c4ccccc4)nn3-c3ccccc3)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(-c2ccccc2)nn(-c2ccccc2)c1O3 ; c1ccc(-c2ccc(N3c4cc(-n5c6ccccc6c6ccccc65)ccc4B4c5c(cc(-n6c7ccccc7c7ccccc76)cc53)Oc3c4c(-c4ccccc4)nn3-c3ccccc3)cc2)cc1]

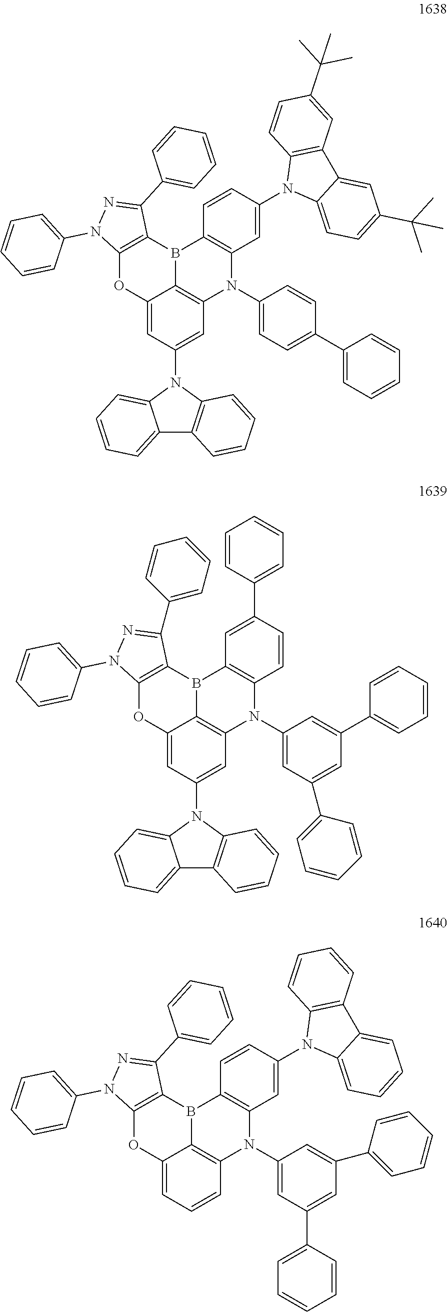 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(-c2ccccc2)nn(-c2ccccc2)c1O3 ; c1ccc(-c2cc(-c3ccccc3)cc(N3c4ccc(-c5ccccc5)cc4B4c5c(cc(-n6c7ccccc7c7ccccc76)cc53)Oc3c4c(-c4ccccc4)nn3-c3ccccc3)c2)cc1 ; c1ccc(-c2cc(-c3ccccc3)cc(N3c4cc(-n5c6ccccc6c6ccccc65)ccc4B4c5c(cccc53)Oc3c4c(-c4ccccc4)nn3-c3ccccc3)c2)cc1]

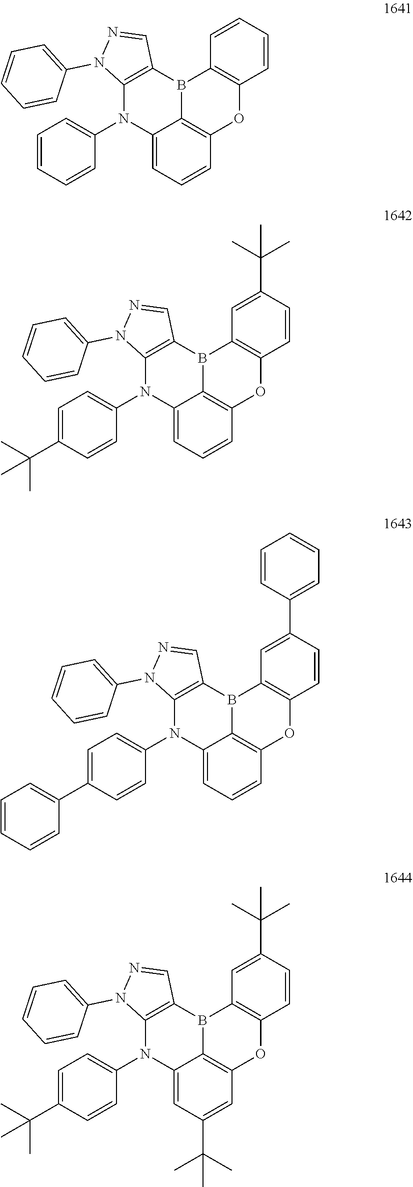

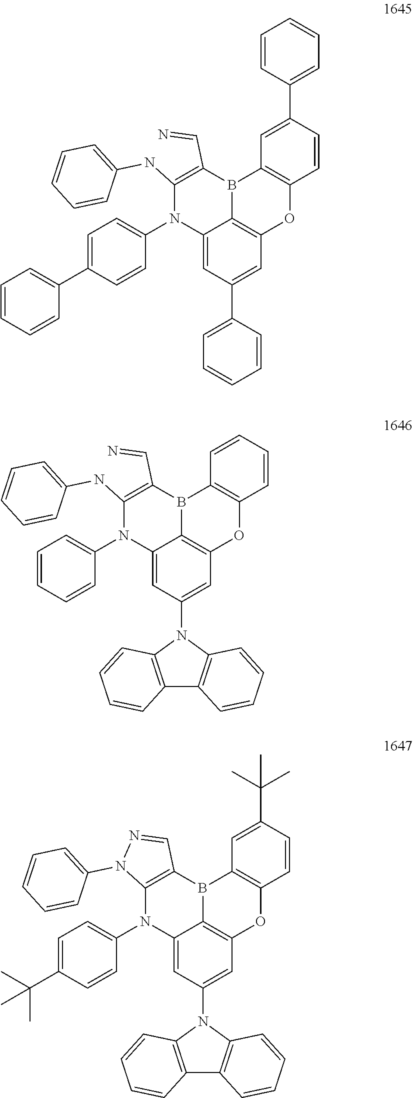 [SMILES: N=CC1=C(Nc2ccccc2)N(c2ccc(-c3ccccc3)cc2)c2cc(-c3ccccc3)cc3c2B1c1cc(-c2ccccc2)ccc1O3 ; N=CC1=C(Nc2ccccc2)N(c2ccccc2)c2cc(-n3c4ccccc4c4ccccc43)cc3c2B1c1ccccc1O3 ; CC(C)(C)c1ccc(N2c3cc(-n4c5ccccc5c5ccccc54)cc4c3B(c3cc(C(C)(C)C)ccc3O4)c3cnn(-c4ccccc4)c32)cc1]

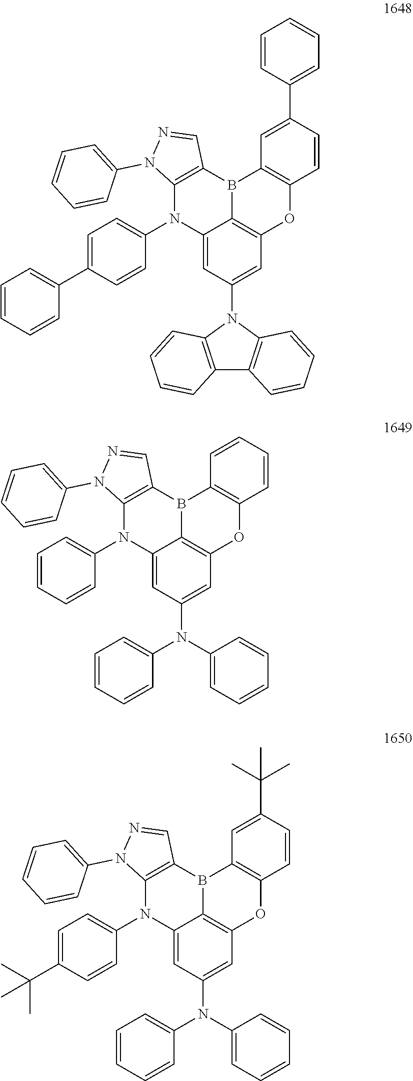 [SMILES: c1ccc(-c2ccc(N3c4cc(-n5c6ccccc6c6ccccc65)cc5c4B(c4cc(-c6ccccc6)ccc4O5)c4cnn(-c5ccccc5)c43)cc2)cc1 ; c1ccc(N(c2ccccc2)c2cc3c4c(c2)N(c2ccccc2)c2c(cnn2-c2ccccc2)B4c2ccccc2O3)cc1 ; CC(C)(C)c1ccc(N2c3cc(N(c4ccccc4)c4ccccc4)cc4c3B(c3cc(C(C)(C)C)ccc3O4)c3cnn(-c4ccccc4)c32)cc1]

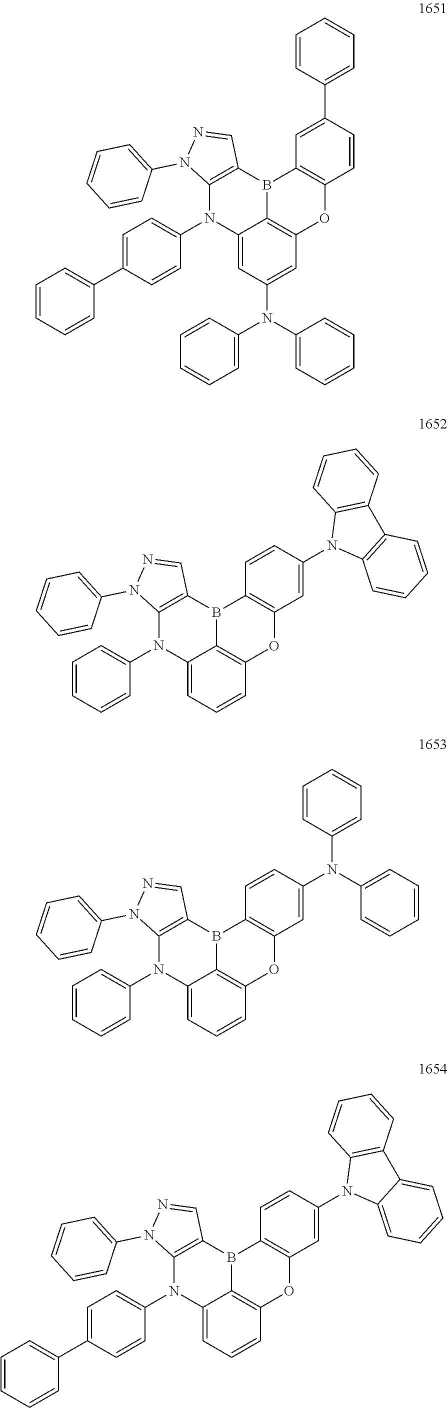 [SMILES: c1ccc(-c2ccc(N3c4cc(N(c5ccccc5)c5ccccc5)cc5c4B(c4cc(-c6ccccc6)ccc4O5)c4cnn(-c5ccccc5)c43)cc2)cc1 ; c1ccc(N2c3cccc4c3B(c3ccc(-n5c6ccccc6c6ccccc65)cc3O4)c3cnn(-c4ccccc4)c32)cc1 ; c1ccc(N(c2ccccc2)c2ccc3c(c2)Oc2cccc4c2B3c2cnn(-c3ccccc3)c2N4c2ccccc2)cc1 ; c1ccc(-c2ccc(N3c4cccc5c4B(c4ccc(-n6c7ccccc7c7ccccc76)cc4O5)c4cnn(-c5ccccc5)c43)cc2)cc1]

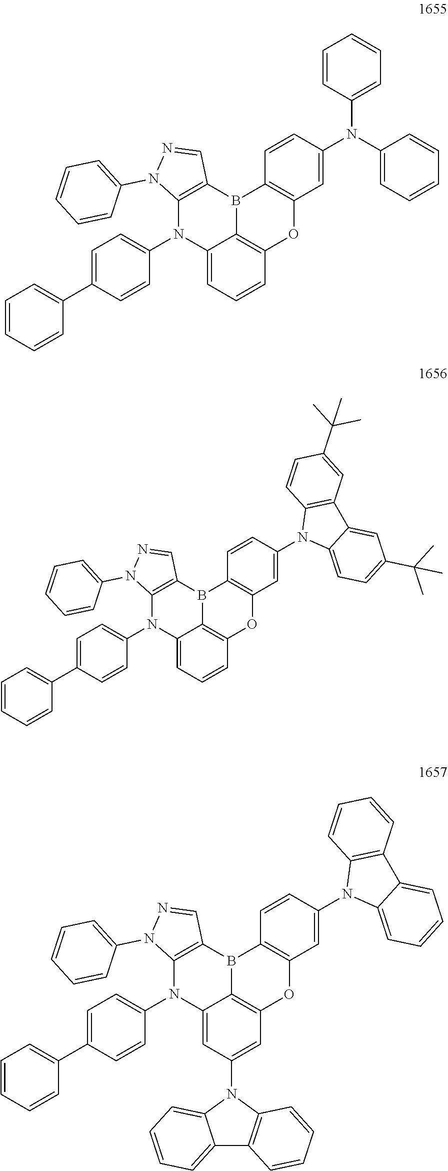 [SMILES: c1ccc(-c2ccc(N3c4cccc5c4B(c4ccc(N(c6ccccc6)c6ccccc6)cc4O5)c4cnn(-c5ccccc5)c43)cc2)cc1 ; CC(C)(C)c1ccc2c(c1)c1cc(C(C)(C)C)ccc1n2-c1ccc2c(c1)Oc1cccc3c1B2c1cnn(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4cnn(-c5ccccc5)c43)cc2)cc1]

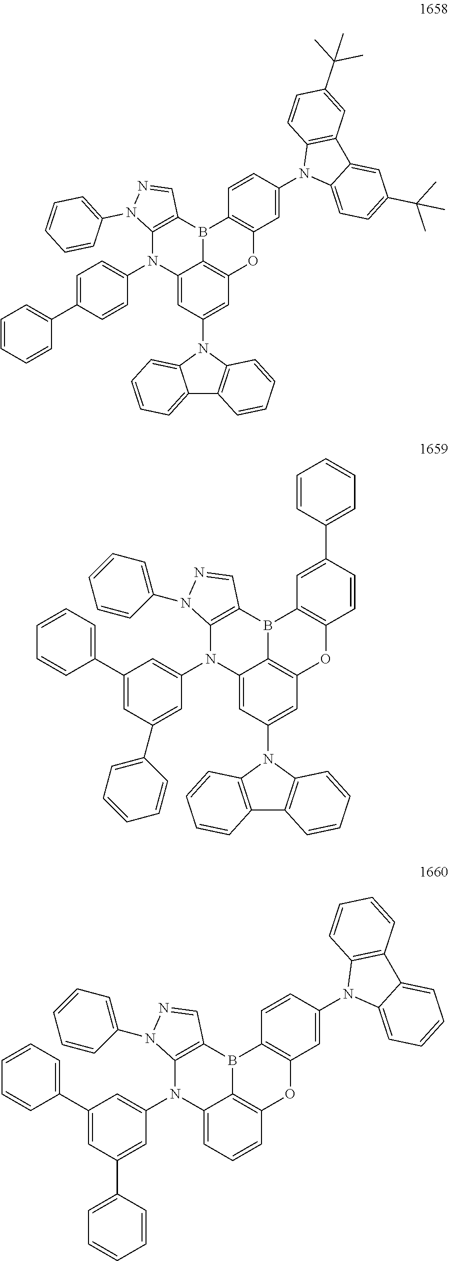 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1cnn(-c2ccccc2)c1N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4cnn(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4cnn(-c5ccccc5)c43)c2)cc1]

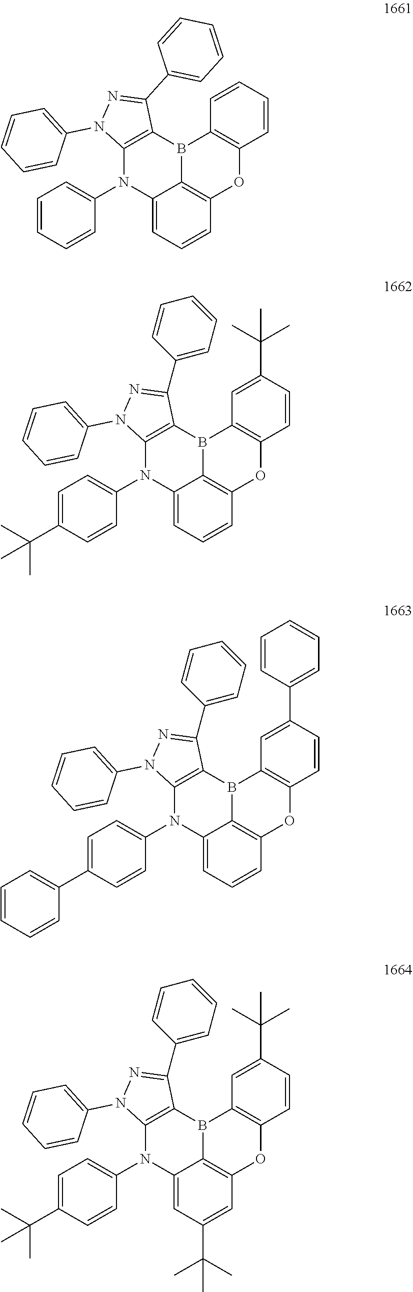

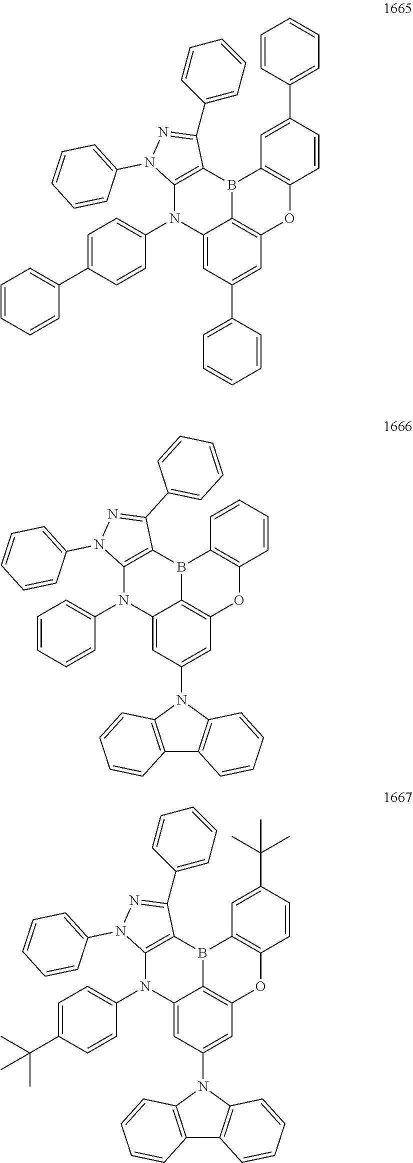

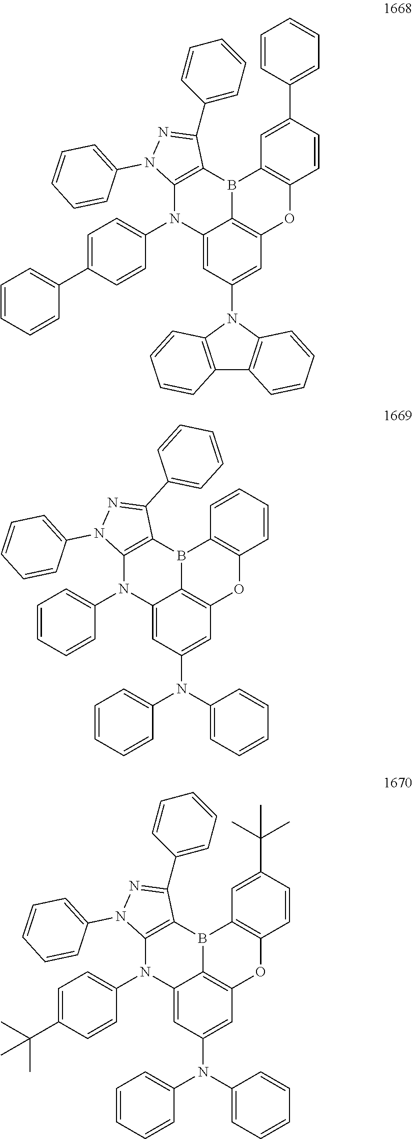

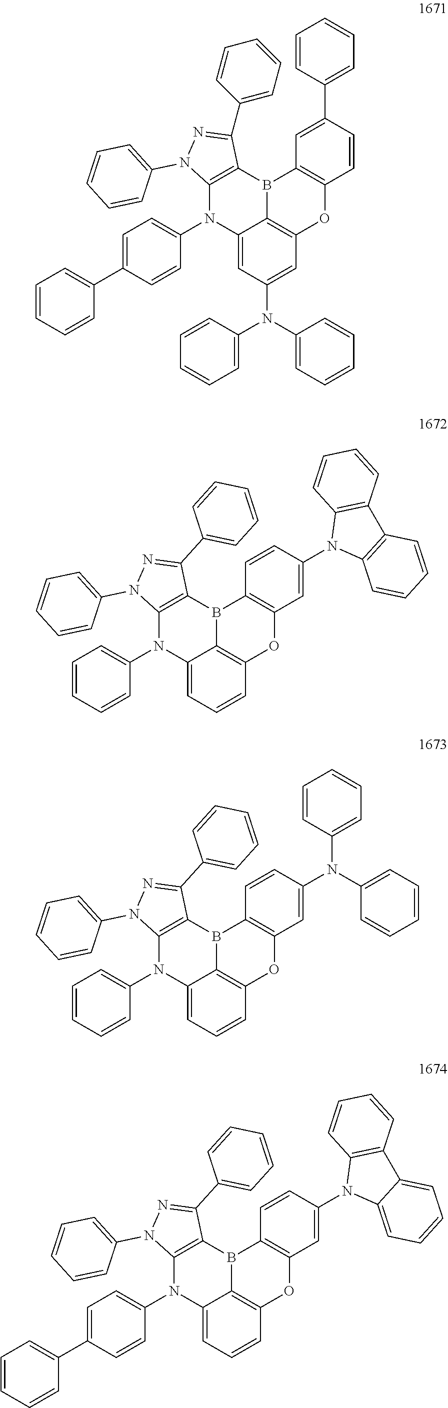 [SMILES: c1ccc(-c2ccc(N3c4cc(N(c5ccccc5)c5ccccc5)cc5c4B(c4cc(-c6ccccc6)ccc4O5)c4c(-c5ccccc5)nn(-c5ccccc5)c43)cc2)cc1 ; c1ccc(-c2nn(-c3ccccc3)c3c2B2c4ccc(-n5c6ccccc6c6ccccc65)cc4Oc4cccc(c42)N3c2ccccc2)cc1 ; c1ccc(-c2nn(-c3ccccc3)c3c2B2c4ccc(N(c5ccccc5)c5ccccc5)cc4Oc4cccc(c42)N3c2ccccc2)cc1 ; c1ccc(-c2ccc(N3c4cccc5c4B(c4ccc(-n6c7ccccc7c7ccccc76)cc4O5)c4c(-c5ccccc5)nn(-c5ccccc5)c43)cc2)cc1]

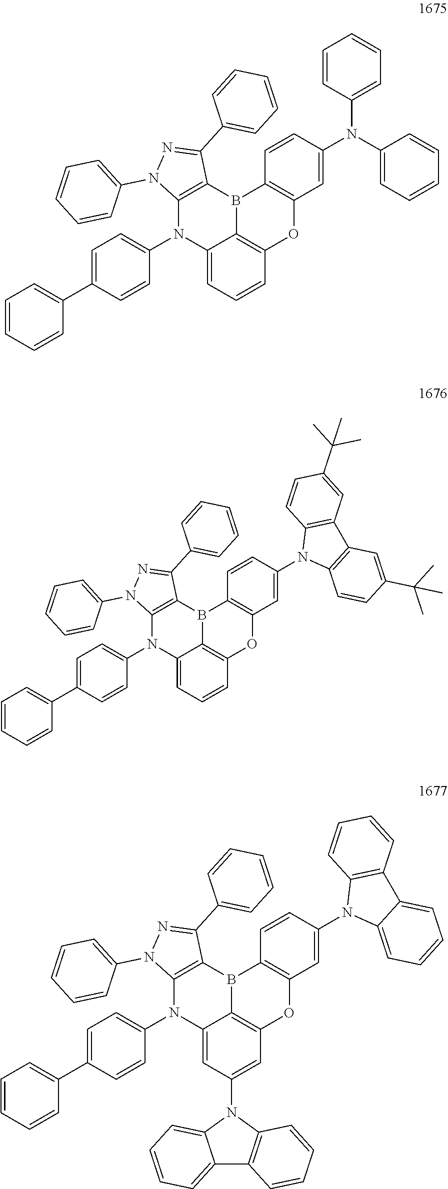 [SMILES: c1ccc(-c2ccc(N3c4cccc5c4B(c4ccc(N(c6ccccc6)c6ccccc6)cc4O5)c4c(-c5ccccc5)nn(-c5ccccc5)c43)cc2)cc1 ; CC(C)(C)c1ccc2c(c1)c1cc(C(C)(C)C)ccc1n2-c1ccc2c(c1)Oc1cccc3c1B2c1c(-c2ccccc2)nn(-c2ccccc2)c1N3c1ccc(-c2ccccc2)cc1 ; c1ccc(-c2ccc(N3c4cc(-n5c6ccccc6c6ccccc65)cc5c4B(c4ccc(-n6c7ccccc7c7ccccc76)cc4O5)c4c(-c5ccccc5)nn(-c5ccccc5)c43)cc2)cc1]

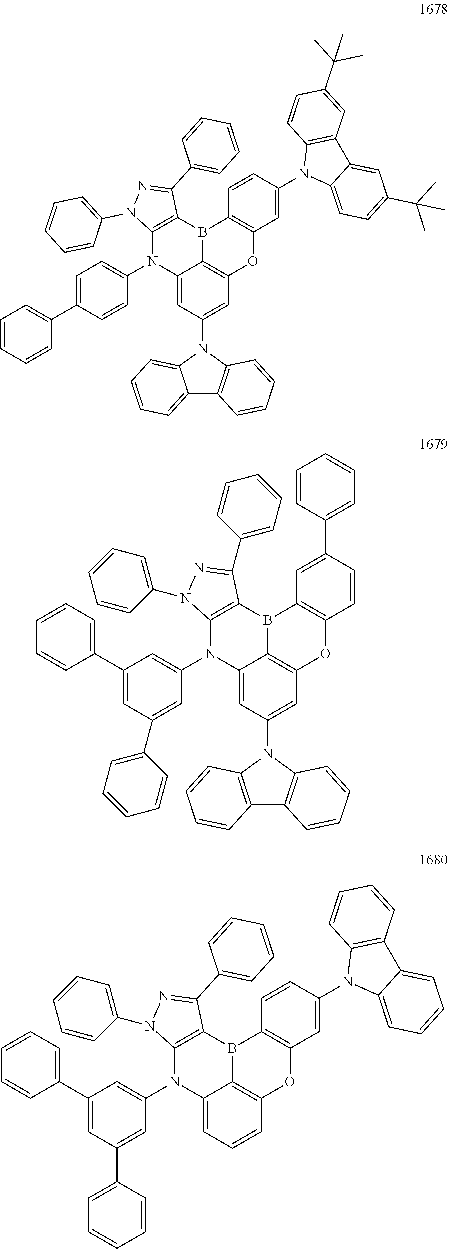 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(-c2ccccc2)nn(-c2ccccc2)c1N3c1ccc(-c2ccccc2)cc1 ; c1ccc(-c2cc(-c3ccccc3)cc(N3c4cc(-n5c6ccccc6c6ccccc65)cc5c4B(c4cc(-c6ccccc6)ccc4O5)c4c(-c5ccccc5)nn(-c5ccccc5)c43)c2)cc1 ; c1ccc(-c2cc(-c3ccccc3)cc(N3c4cccc5c4B(c4ccc(-n6c7ccccc7c7ccccc76)cc4O5)c4c(-c5ccccc5)nn(-c5ccccc5)c43)c2)cc1]

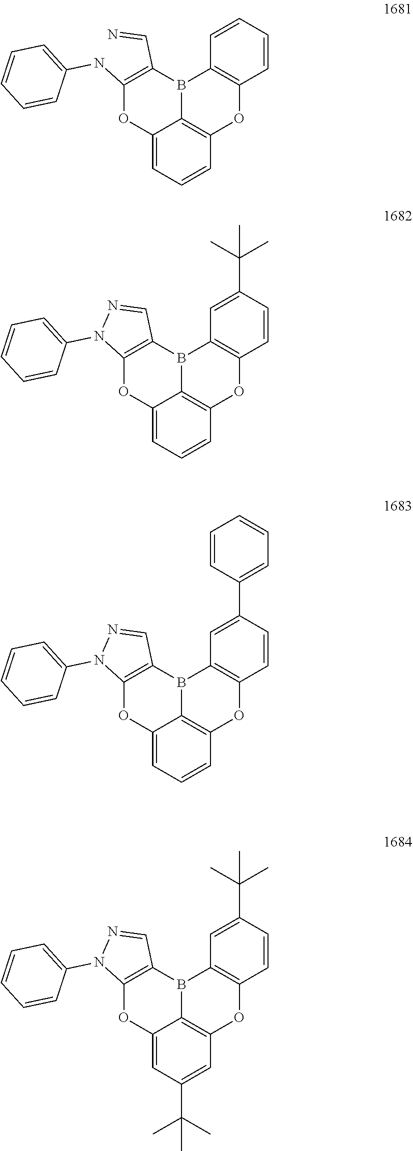 [SMILES: N=CC1=C(Nc2ccccc2)Oc2cccc3c2B1c1ccccc1O3 ; CC(C)(C)c1ccc2c(c1)B1c3cnn(-c4ccccc4)c3Oc3cccc(c31)O2 ; c1ccc(-c2ccc3c(c2)B2c4cnn(-c5ccccc5)c4Oc4cccc(c42)O3)cc1 ; CC(C)(C)c1cc2c3c(c1)Oc1c(cnn1-c1ccccc1)B3c1cc(C(C)(C)C)ccc1O2]

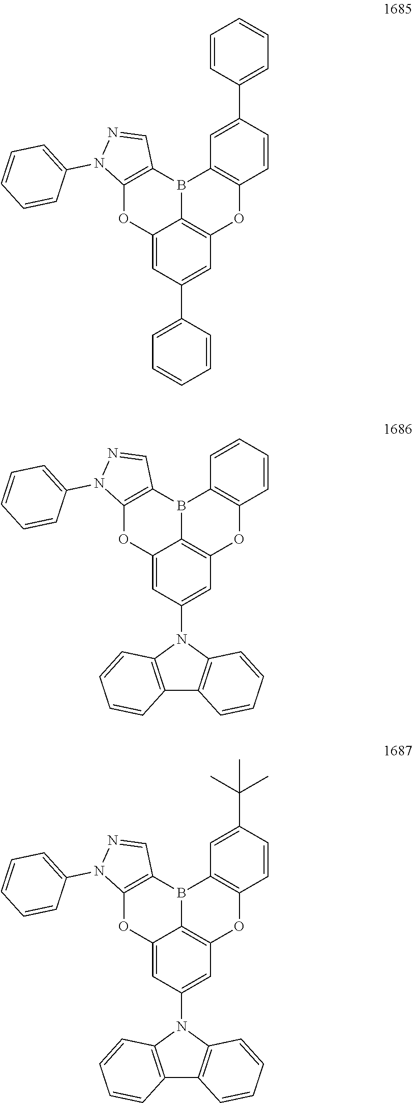 [SMILES: c1ccc(-c2cc3c4c(c2)Oc2c(cnn2-c2ccccc2)B4c2cc(-c4ccccc4)ccc2O3)cc1 ; c1ccc(-n2ncc3c2Oc2cc(-n4c5ccccc5c5ccccc54)cc4c2B3c2ccccc2O4)cc1 ; CC(C)(C)c1ccc2c(c1)B1c3cnn(-c4ccccc4)c3Oc3cc(-n4c5ccccc5c5ccccc54)cc(c31)O2]

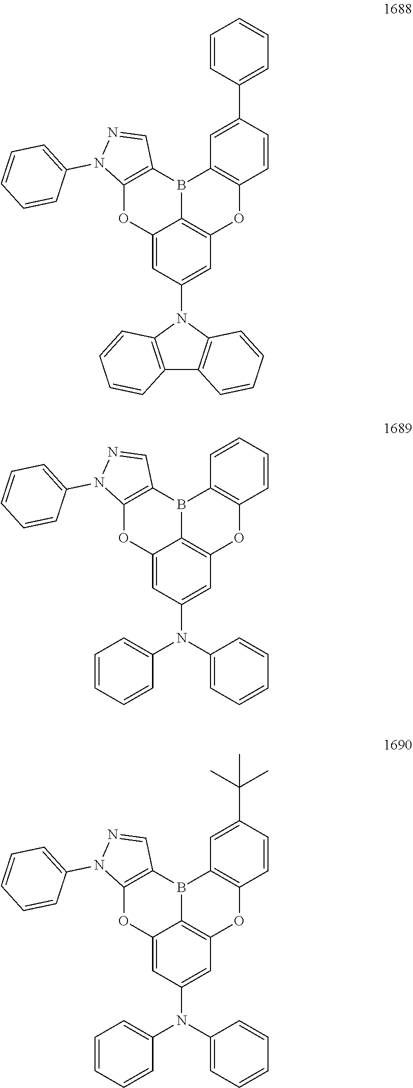 [SMILES: c1ccc(-c2ccc3c(c2)B2c4cnn(-c5ccccc5)c4Oc4cc(-n5c6ccccc6c6ccccc65)cc(c42)O3)cc1 ; c1ccc(N(c2ccccc2)c2cc3c4c(c2)Oc2c(cnn2-c2ccccc2)B4c2ccccc2O3)cc1 ; CC(C)(C)c1ccc2c(c1)B1c3cnn(-c4ccccc4)c3Oc3cc(N(c4ccccc4)c4ccccc4)cc(c31)O2]

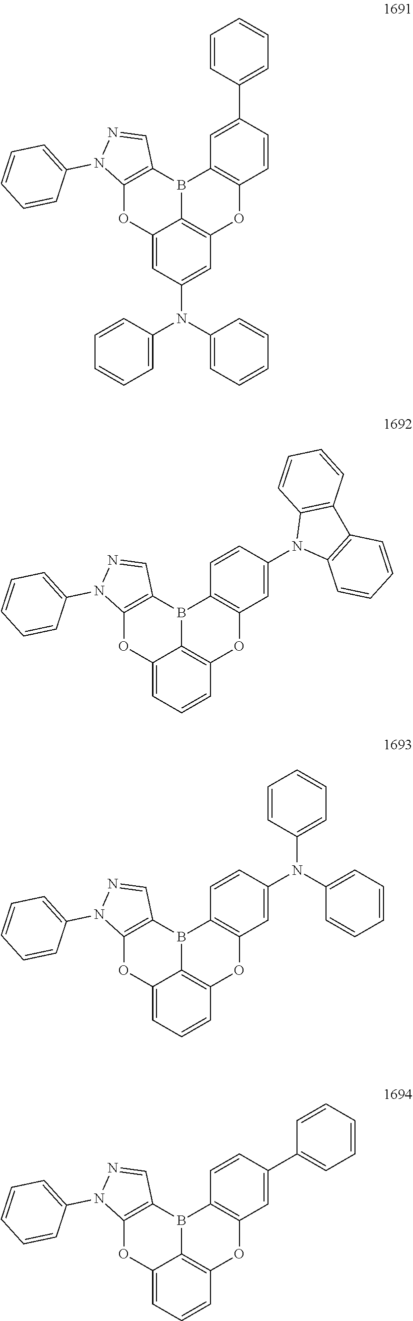 [SMILES: c1ccc(-c2ccc3c(c2)B2c4cnn(-c5ccccc5)c4Oc4cc(N(c5ccccc5)c5ccccc5)cc(c42)O3)cc1 ; c1ccc(-n2ncc3c2Oc2cccc4c2B3c2ccc(-n3c5ccccc5c5ccccc53)cc2O4)cc1 ; c1ccc(N(c2ccccc2)c2ccc3c(c2)Oc2cccc4c2B3c2cnn(-c3ccccc3)c2O4)cc1 ; c1ccc(-c2ccc3c(c2)Oc2cccc4c2B3c2cnn(-c3ccccc3)c2O4)cc1]

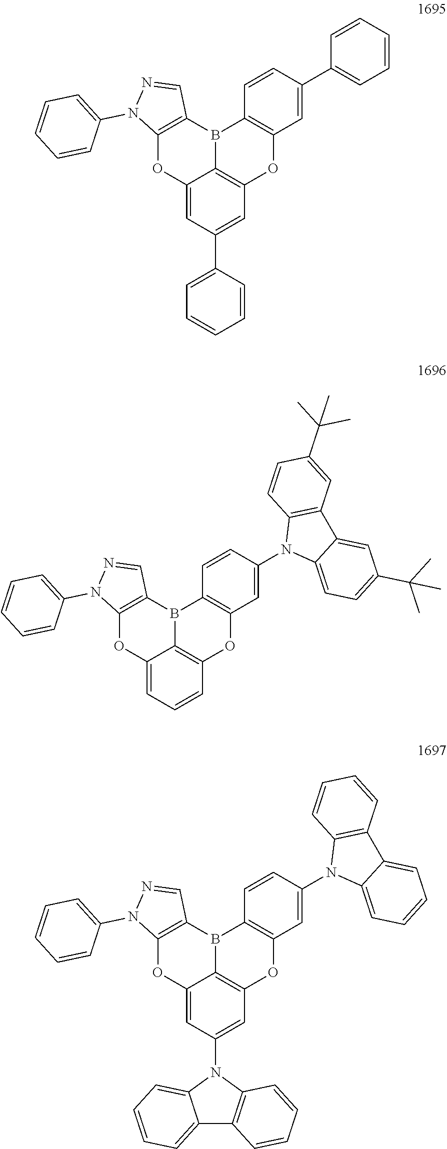 [SMILES: c1ccc(-c2ccc3c(c2)Oc2cc(-c4ccccc4)cc4c2B3c2cnn(-c3ccccc3)c2O4)cc1 ; CC(C)(C)c1ccc2c(c1)c1cc(C(C)(C)C)ccc1n2-c1ccc2c(c1)Oc1cccc3c1B2c1cnn(-c2ccccc2)c1O3 ; c1ccc(-n2ncc3c2Oc2cc(-n4c5ccccc5c5ccccc54)cc4c2B3c2ccc(-n3c5ccccc5c5ccccc53)cc2O4)cc1]

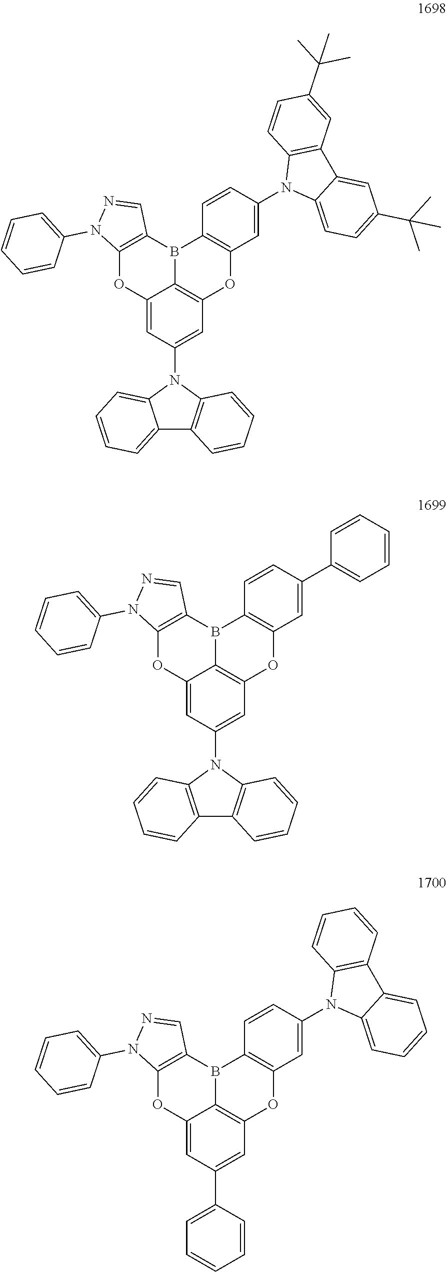 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1cnn(-c2ccccc2)c1O3 ; c1ccc(-c2ccc3c(c2)Oc2cc(-n4c5ccccc5c5ccccc54)cc4c2B3c2cnn(-c3ccccc3)c2O4)cc1 ; c1ccc(-c2cc3c4c(c2)Oc2c(cnn2-c2ccccc2)B4c2ccc(-n4c5ccccc5c5ccccc54)cc2O3)cc1]

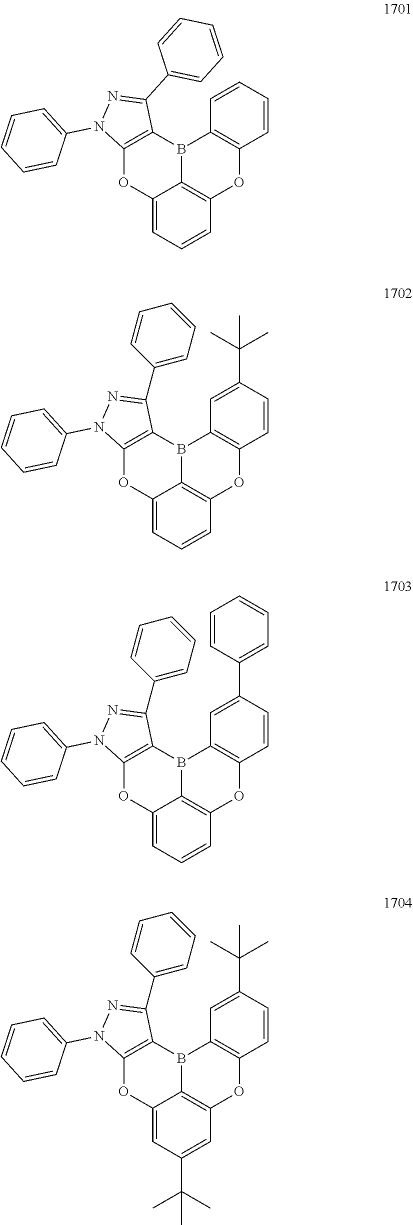 [SMILES: c1ccc(-c2nn(-c3ccccc3)c3c2B2c4ccccc4Oc4cccc(c42)O3)cc1 ; CC(C)(C)c1ccc2c(c1)B1c3c(cccc3Oc3c1c(-c1ccccc1)nn3-c1ccccc1)O2 ; c1ccc(-c2ccc3c(c2)B2c4c(cccc4Oc4c2c(-c2ccccc2)nn4-c2ccccc2)O3)cc1 ; CC(C)(C)c1cc2c3c(c1)Oc1c(c(-c4ccccc4)nn1-c1ccccc1)B3c1cc(C(C)(C)C)ccc1O2]

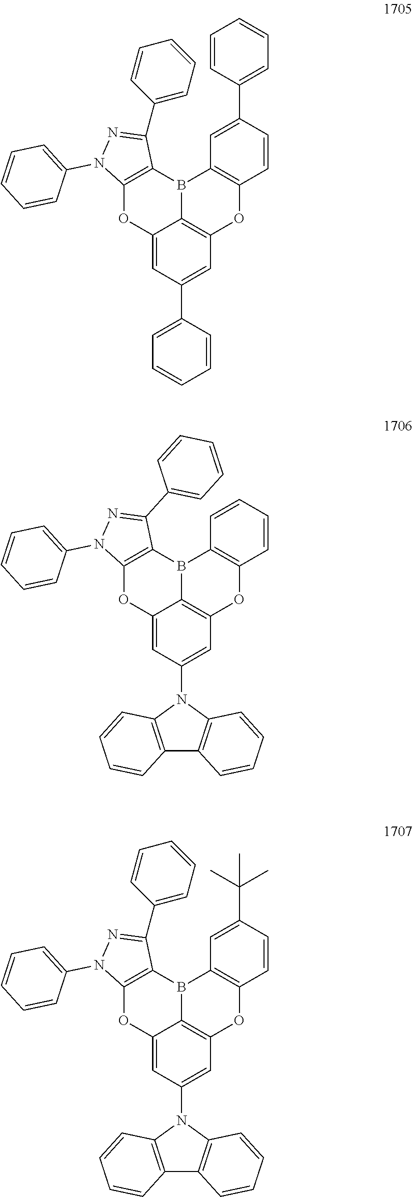 [SMILES: c1ccc(-c2cc3c4c(c2)Oc2c(c(-c5ccccc5)nn2-c2ccccc2)B4c2cc(-c4ccccc4)ccc2O3)cc1 ; c1ccc(-c2nn(-c3ccccc3)c3c2B2c4ccccc4Oc4cc(-n5c6ccccc6c6ccccc65)cc(c42)O3)cc1 ; CC(C)(C)c1ccc2c(c1)B1c3c(cc(-n4c5ccccc5c5ccccc54)cc3Oc3c1c(-c1ccccc1)nn3-c1ccccc1)O2]

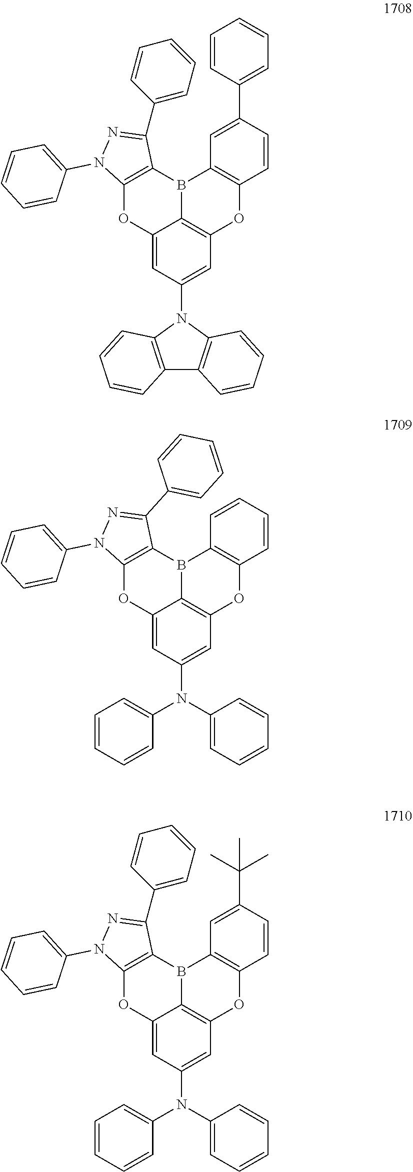 [SMILES: c1ccc(-c2ccc3c(c2)B2c4c(cc(-n5c6ccccc6c6ccccc65)cc4Oc4c2c(-c2ccccc2)nn4-c2ccccc2)O3)cc1 ; c1ccc(-c2nn(-c3ccccc3)c3c2B2c4ccccc4Oc4cc(N(c5ccccc5)c5ccccc5)cc(c42)O3)cc1 ; CC(C)(C)c1ccc2c(c1)B1c3c(cc(N(c4ccccc4)c4ccccc4)cc3Oc3c1c(-c1ccccc1)nn3-c1ccccc1)O2]

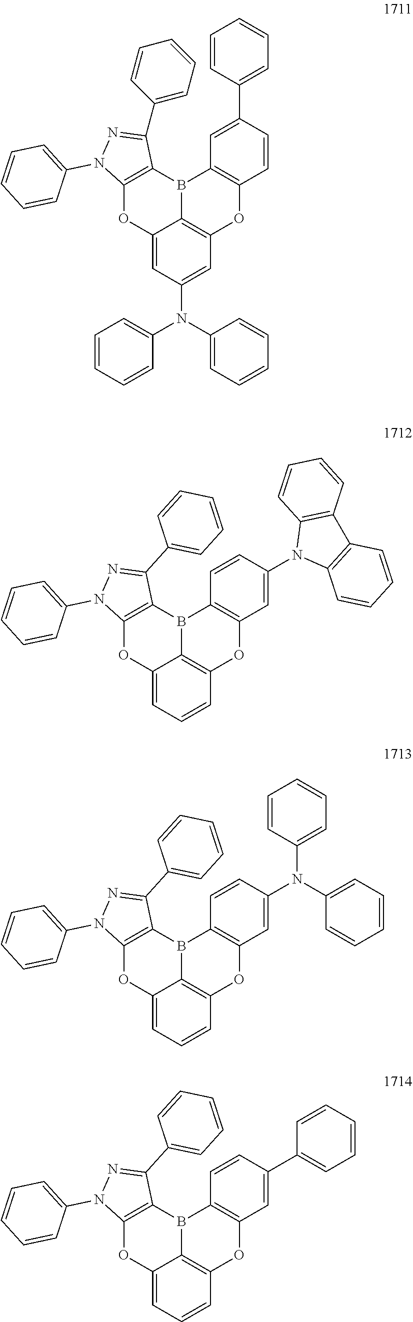

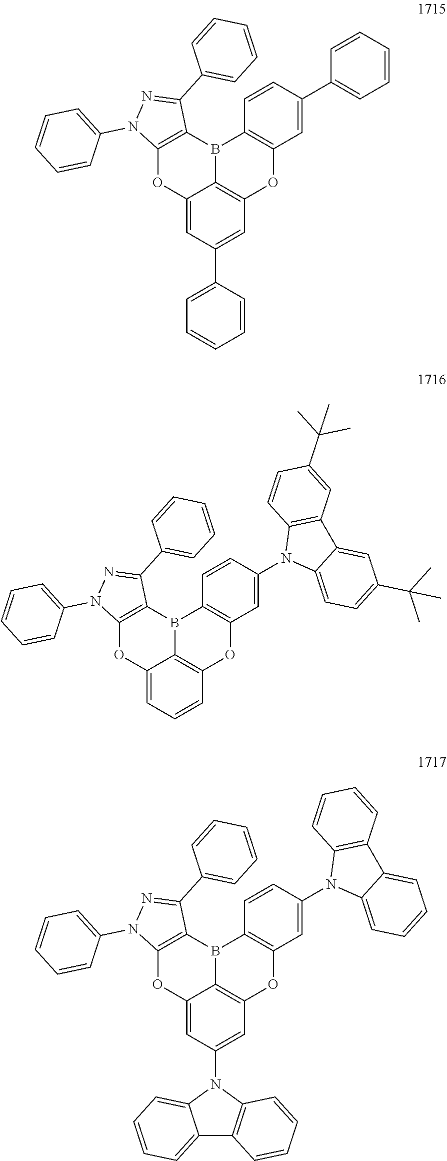 [SMILES: c1ccc(-c2ccc3c(c2)Oc2cc(-c4ccccc4)cc4c2B3c2c(-c3ccccc3)nn(-c3ccccc3)c2O4)cc1 ; CC(C)(C)c1ccc2c(c1)c1cc(C(C)(C)C)ccc1n2-c1ccc2c(c1)Oc1cccc3c1B2c1c(-c2ccccc2)nn(-c2ccccc2)c1O3 ; c1ccc(-c2nn(-c3ccccc3)c3c2B2c4ccc(-n5c6ccccc6c6ccccc65)cc4Oc4cc(-n5c6ccccc6c6ccccc65)cc(c42)O3)cc1]

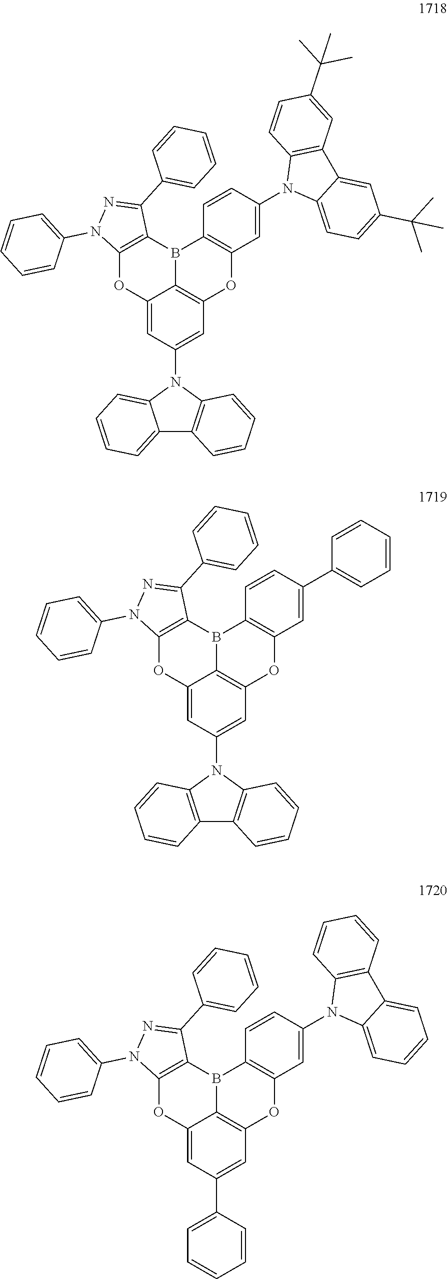 [SMILES: CC(C)(C)c1ccc2c(c1)c1cc(C(C)(C)C)ccc1n2-c1ccc2c(c1)Oc1cc(-n3c4ccccc4c4ccccc43)cc3c1B2c1c(-c2ccccc2)nn(-c2ccccc2)c1O3 ; c1ccc(-c2ccc3c(c2)Oc2cc(-n4c5ccccc5c5ccccc54)cc4c2B3c2c(-c3ccccc3)nn(-c3ccccc3)c2O4)cc1 ; c1ccc(-c2cc3c4c(c2)Oc2c(c(-c5ccccc5)nn2-c2ccccc2)B4c2ccc(-n4c5ccccc5c5ccccc54)cc2O3)cc1]

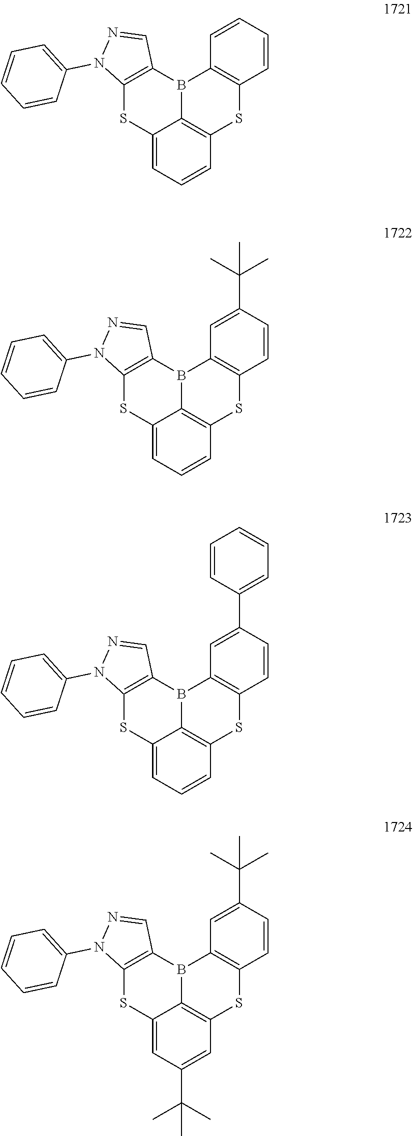 [SMILES: c1ccc(-n2ncc3c2Sc2cccc4c2B3c2ccccc2S4)cc1 ; CC(C)(C)c1ccc2c(c1)B1c3cnn(-c4ccccc4)c3Sc3cccc(c31)S2 ; c1ccc(-c2ccc3c(c2)B2c4cnn(-c5ccccc5)c4Sc4cccc(c42)S3)cc1 ; CC(C)(C)c1cc2c3c(c1)Sc1c(cnn1-c1ccccc1)B3c1cc(C(C)(C)C)ccc1S2]

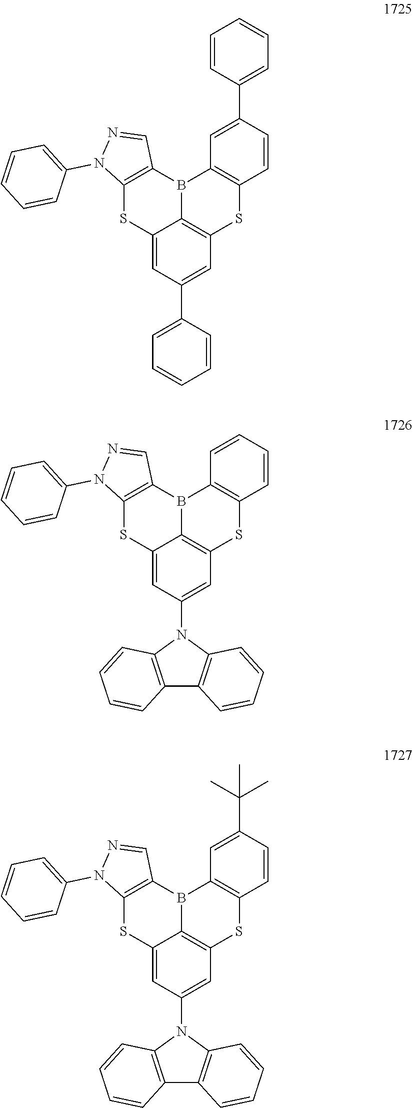 [SMILES: c1ccc(-c2cc3c4c(c2)Sc2c(cnn2-c2ccccc2)B4c2cc(-c4ccccc4)ccc2S3)cc1 ; c1ccc(-n2ncc3c2Sc2cc(-n4c5ccccc5c5ccccc54)cc4c2B3c2ccccc2S4)cc1 ; CC(C)(C)c1ccc2c(c1)B1c3cnn(-c4ccccc4)c3Sc3cc(-n4c5ccccc5c5ccccc54)cc(c31)S2]

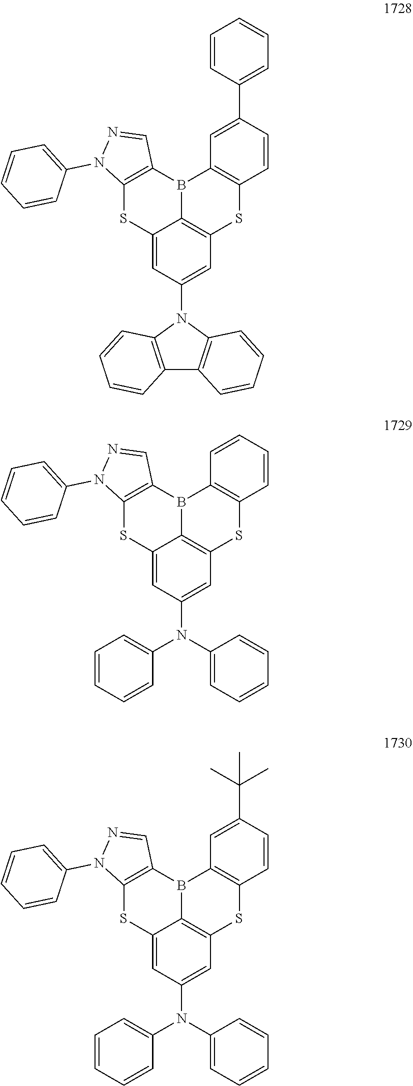

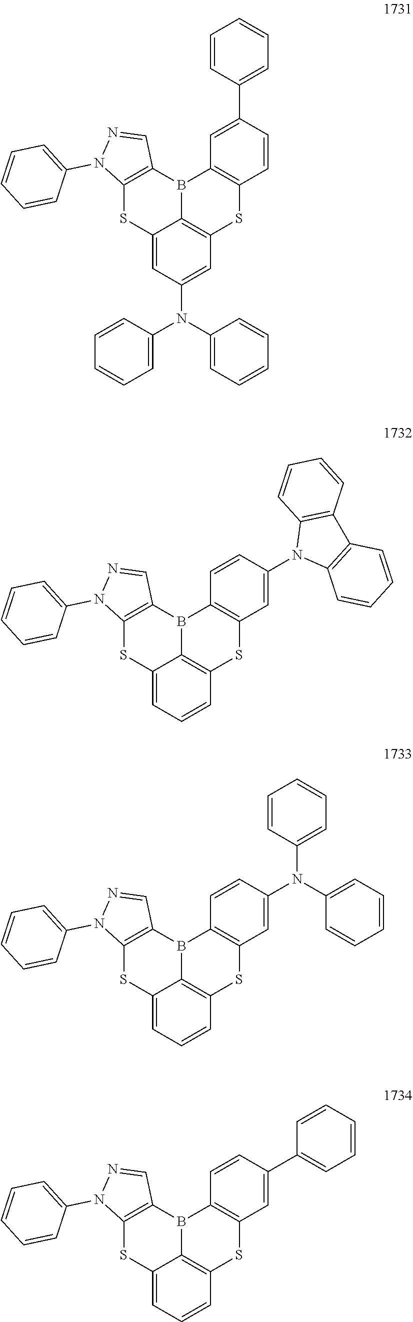 [SMILES: c1ccc(-c2ccc3c(c2)B2c4cnn(-c5ccccc5)c4Sc4cc(N(c5ccccc5)c5ccccc5)cc(c42)S3)cc1 ; c1ccc(-n2ncc3c2Sc2cccc4c2B3c2ccc(-n3c5ccccc5c5ccccc53)cc2S4)cc1 ; c1ccc(N(c2ccccc2)c2ccc3c(c2)Sc2cccc4c2B3c2cnn(-c3ccccc3)c2S4)cc1 ; c1ccc(-c2ccc3c(c2)Sc2cccc4c2B3c2cnn(-c3ccccc3)c2S4)cc1]

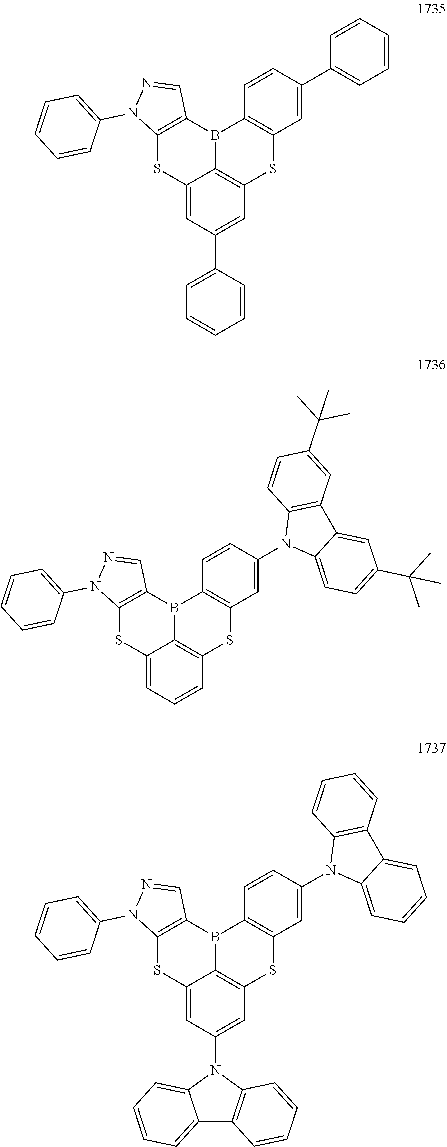 [SMILES: c1ccc(-c2ccc3c(c2)Sc2cc(-c4ccccc4)cc4c2B3c2cnn(-c3ccccc3)c2S4)cc1 ; CC(C)(C)c1ccc2c(c1)c1cc(C(C)(C)C)ccc1n2-c1ccc2c(c1)Sc1cccc3c1B2c1cnn(-c2ccccc2)c1S3 ; c1ccc(-n2ncc3c2Sc2cc(-n4c5ccccc5c5ccccc54)cc4c2B3c2ccc(-n3c5ccccc5c5ccccc53)cc2S4)cc1]

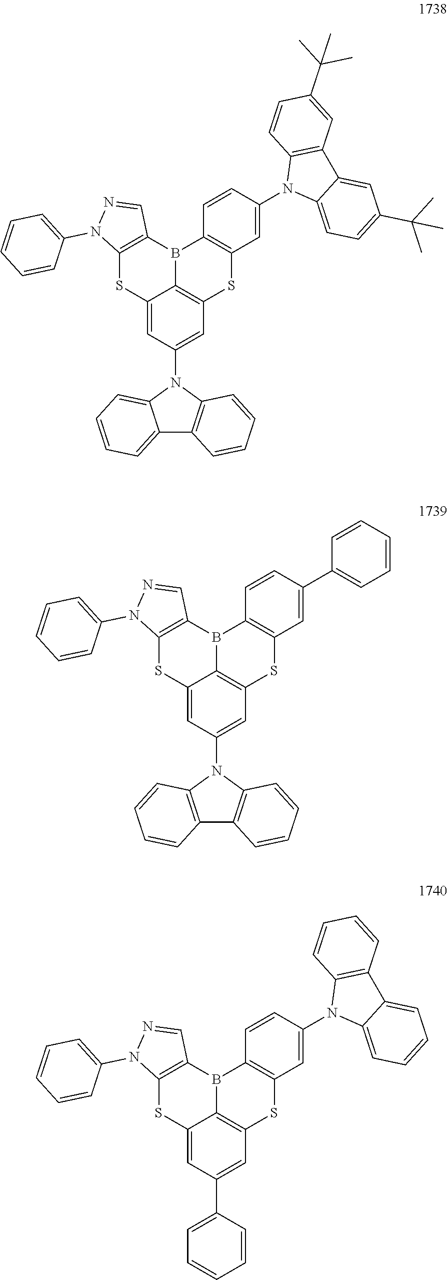 [SMILES: CC(C)(C)c1ccc2c(c1)c1cc(C(C)(C)C)ccc1n2-c1ccc2c(c1)Sc1cc(-n3c4ccccc4c4ccccc43)cc3c1B2c1cnn(-c2ccccc2)c1S3 ; c1ccc(-c2ccc3c(c2)Sc2cc(-n4c5ccccc5c5ccccc54)cc4c2B3c2cnn(-c3ccccc3)c2S4)cc1 ; c1ccc(-c2cc3c4c(c2)Sc2c(cnn2-c2ccccc2)B4c2ccc(-n4c5ccccc5c5ccccc54)cc2S3)cc1]

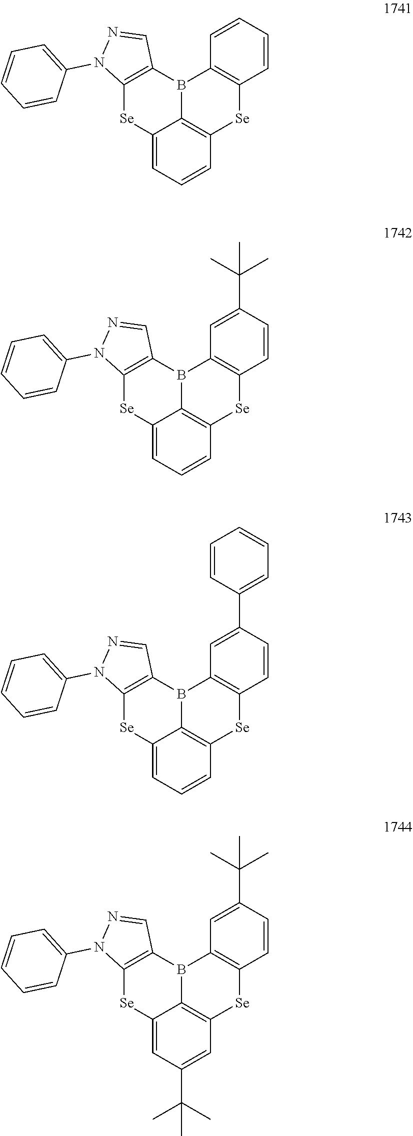 [SMILES: c1ccc(-n2ncc3c2[Se]c2cccc4c2B3c2ccccc2[Se]4)cc1 ; CC(C)(C)c1ccc2c(c1)B1c3cnn(-c4ccccc4)c3[Se]c3cccc(c31)[Se]2 ; c1ccc(-c2ccc3c(c2)B2c4cnn(-c5ccccc5)c4[Se]c4cccc(c42)[Se]3)cc1 ; CC(C)(C)c1cc2c3c(c1)[Se]c1c(cnn1-c1ccccc1)B3c1cc(C(C)(C)C)ccc1[Se]2]

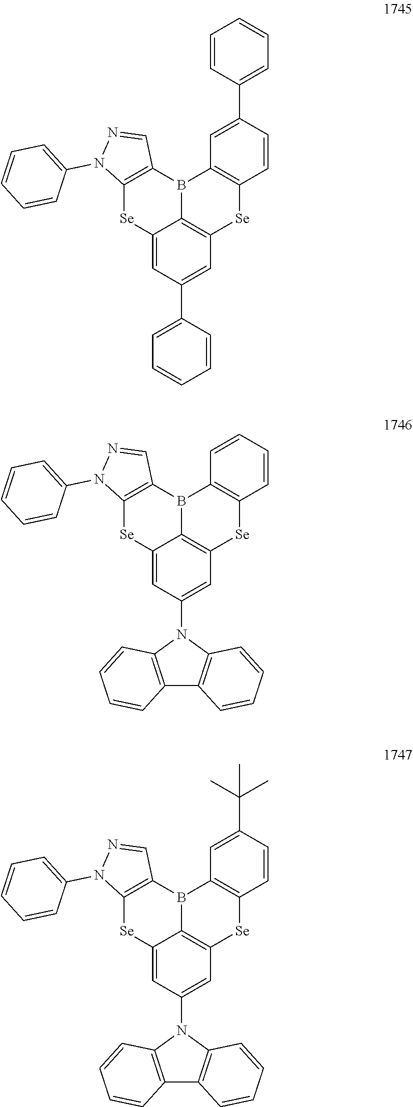 [SMILES: c1ccc(-c2cc3c4c(c2)[Se]c2c(cnn2-c2ccccc2)B4c2cc(-c4ccccc4)ccc2[Se]3)cc1 ; c1ccc(-n2ncc3c2[Se]c2cc(-n4c5ccccc5c5ccccc54)cc4c2B3c2ccccc2[Se]4)cc1 ; CC(C)(C)c1ccc2c(c1)B1c3cnn(-c4ccccc4)c3[Se]c3cc(-n4c5ccccc5c5ccccc54)cc(c31)[Se]2]

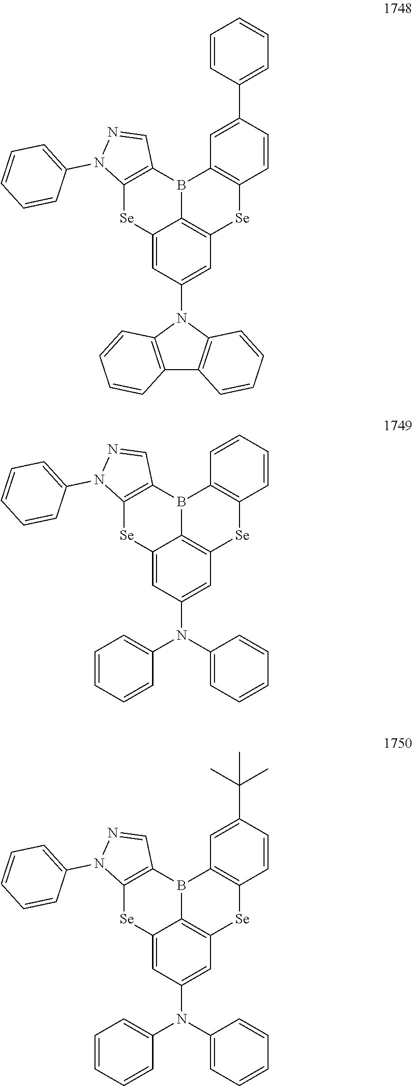

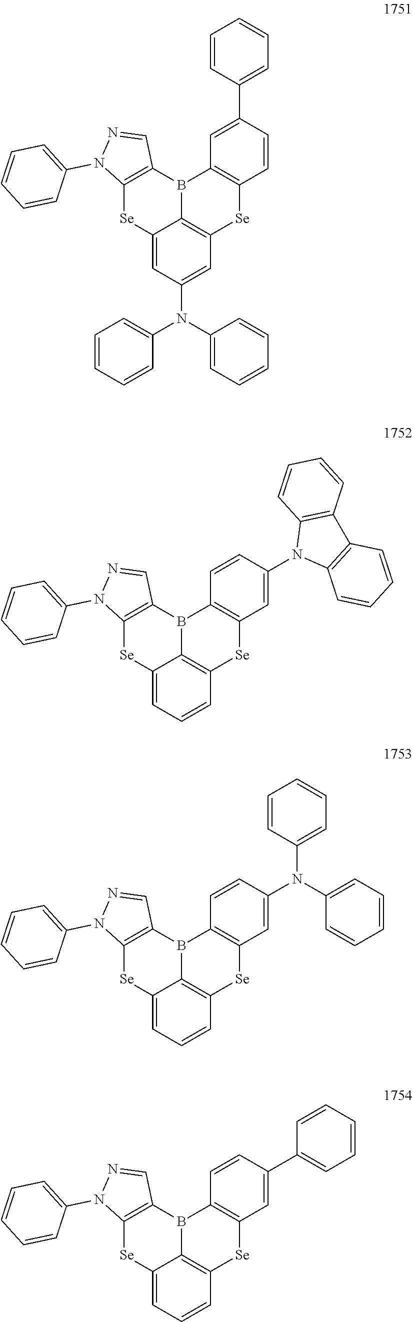 [SMILES: c1ccc(-c2ccc3c(c2)B2c4cnn(-c5ccccc5)c4[Se]c4cc(N(c5ccccc5)c5ccccc5)cc(c42)[Se]3)cc1 ; c1ccc(-n2ncc3c2[Se]c2cccc4c2B3c2ccc(-n3c5ccccc5c5ccccc53)cc2[Se]4)cc1 ; c1ccc(N(c2ccccc2)c2ccc3c(c2)[Se]c2cccc4c2B3c2cnn(-c3ccccc3)c2[Se]4)cc1 ; c1ccc(-c2ccc3c(c2)[Se]c2cccc4c2B3c2cnn(-c3ccccc3)c2[Se]4)cc1]

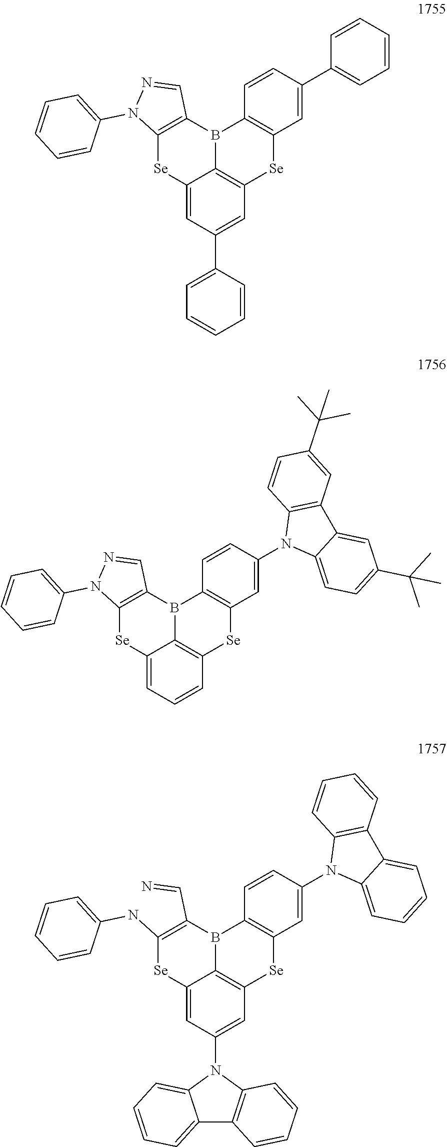 [SMILES: c1ccc(-c2ccc3c(c2)[Se]c2cc(-c4ccccc4)cc4c2B3c2cnn(-c3ccccc3)c2[Se]4)cc1 ; CC(C)(C)c1ccc2c(c1)c1cc(C(C)(C)C)ccc1n2-c1ccc2c(c1)[Se]c1cccc3c1B2c1cnn(-c2ccccc2)c1[Se]3 ; N=CC1=C(Nc2ccccc2)[Se]c2cc(-n3c4ccccc4c4ccccc43)cc3c2B1c1ccc(-n2c4ccccc4c4ccccc42)cc1[Se]3]

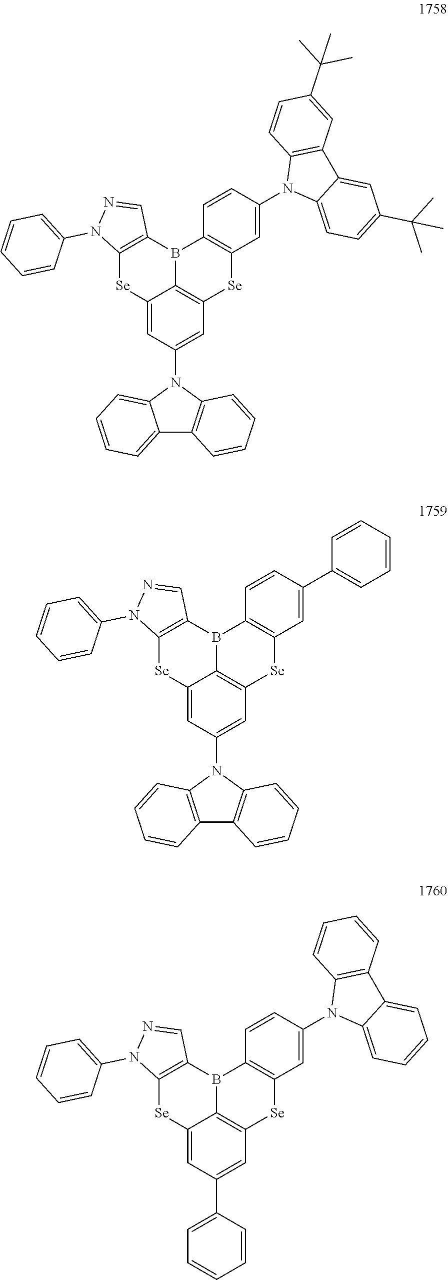 [SMILES: CC(C)(C)c1ccc2c(c1)c1cc(C(C)(C)C)ccc1n2-c1ccc2c(c1)[Se]c1cc(-n3c4ccccc4c4ccccc43)cc3c1B2c1cnn(-c2ccccc2)c1[Se]3 ; c1ccc(-c2ccc3c(c2)[Se]c2cc(-n4c5ccccc5c5ccccc54)cc4c2B3c2cnn(-c3ccccc3)c2[Se]4)cc1 ; c1ccc(-c2cc3c4c(c2)[Se]c2c(cnn2-c2ccccc2)B4c2ccc(-n4c5ccccc5c5ccccc54)cc2[Se]3)cc1]

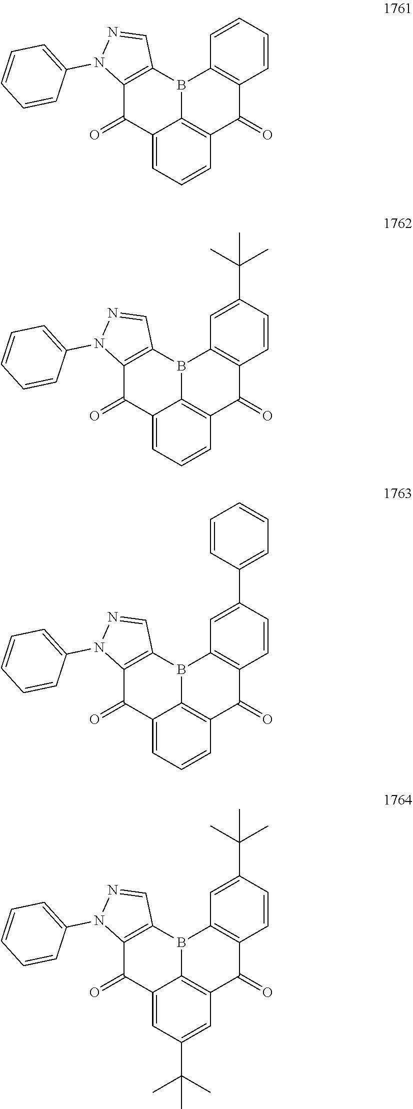 [SMILES: O=C1c2ccccc2B2c3cnn(-c4ccccc4)c3C(=O)c3cccc1c32 ; CC(C)(C)c1ccc2c(c1)B1c3cnn(-c4ccccc4)c3C(=O)c3cccc(c31)C2=O ; O=C1c2ccc(-c3ccccc3)cc2B2c3cnn(-c4ccccc4)c3C(=O)c3cccc1c32 ; CC(C)(C)c1ccc2c(c1)B1c3cnn(-c4ccccc4)c3C(=O)c3cc(C(C)(C)C)cc(c31)C2=O]

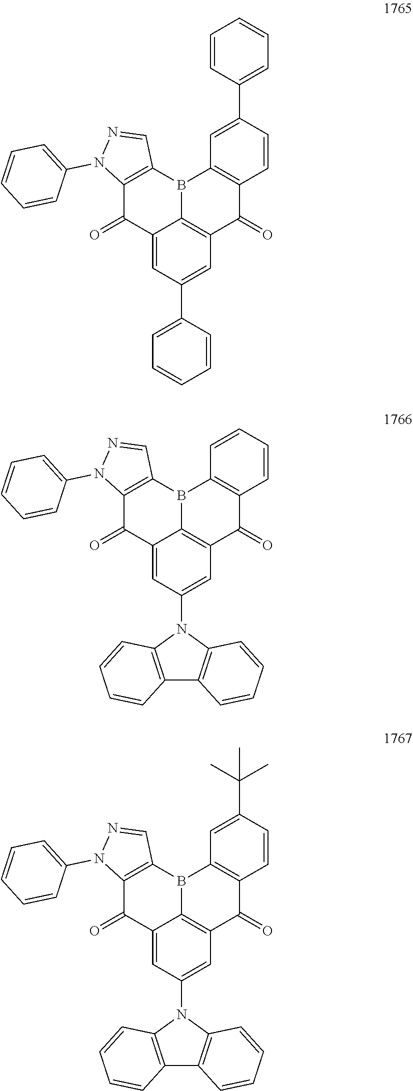

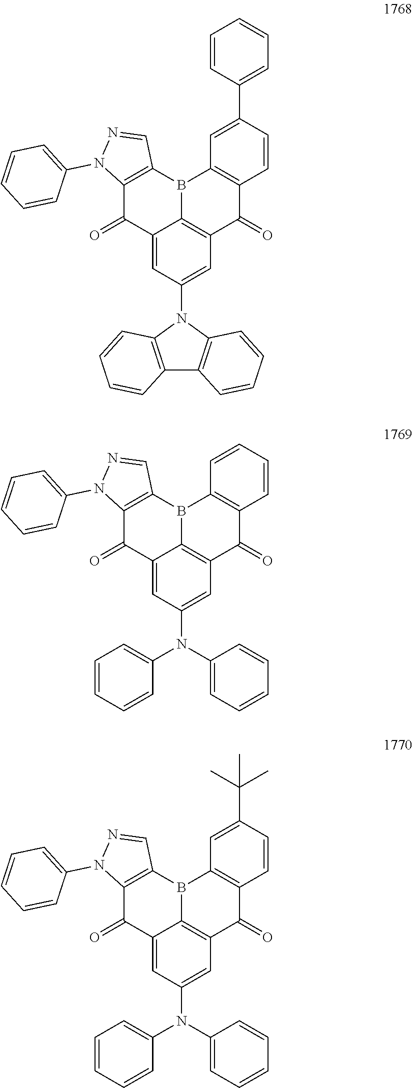

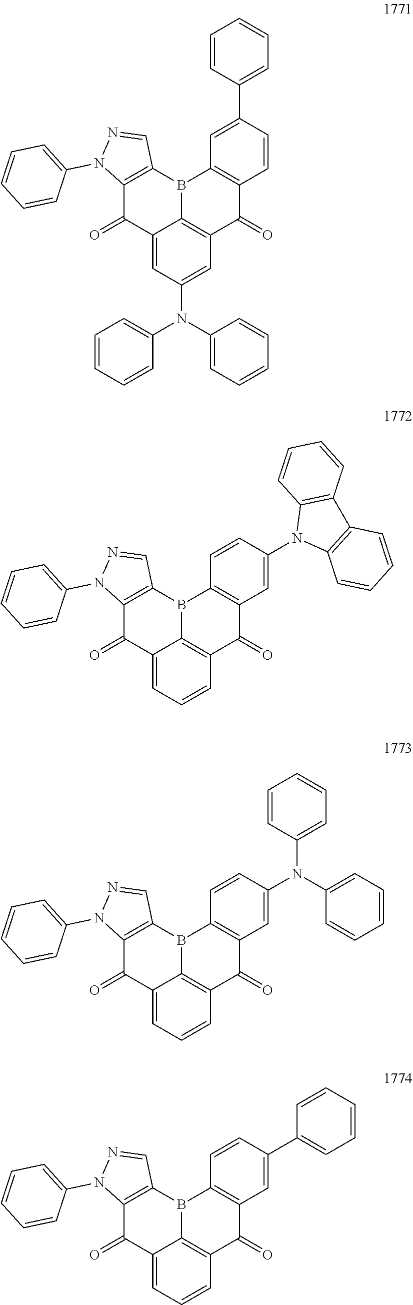 [SMILES: O=C1c2ccc(-c3ccccc3)cc2B2c3cnn(-c4ccccc4)c3C(=O)c3cc(N(c4ccccc4)c4ccccc4)cc1c32 ; O=C1c2cc(-n3c4ccccc4c4ccccc43)ccc2B2c3cnn(-c4ccccc4)c3C(=O)c3cccc1c32 ; O=C1c2cc(N(c3ccccc3)c3ccccc3)ccc2B2c3cnn(-c4ccccc4)c3C(=O)c3cccc1c32 ; O=C1c2cc(-c3ccccc3)ccc2B2c3cnn(-c4ccccc4)c3C(=O)c3cccc1c32]

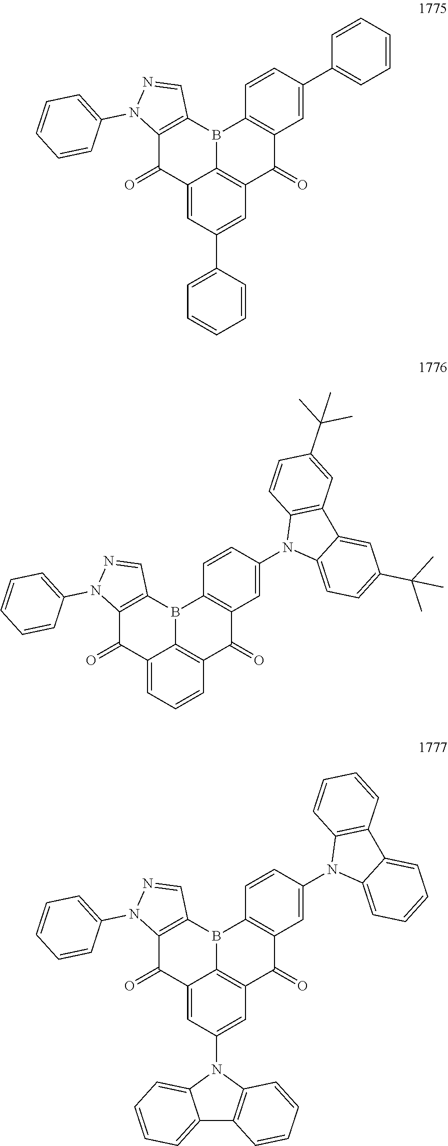 [SMILES: O=C1c2cc(-c3ccccc3)ccc2B2c3cnn(-c4ccccc4)c3C(=O)c3cc(-c4ccccc4)cc1c32 ; CC(C)(C)c1ccc2c(c1)c1cc(C(C)(C)C)ccc1n2-c1ccc2c(c1)C(=O)c1cccc3c1B2c1cnn(-c2ccccc2)c1C3=O ; O=C1c2cc(-n3c4ccccc4c4ccccc43)ccc2B2c3cnn(-c4ccccc4)c3C(=O)c3cc(-n4c5ccccc5c5ccccc54)cc1c32]

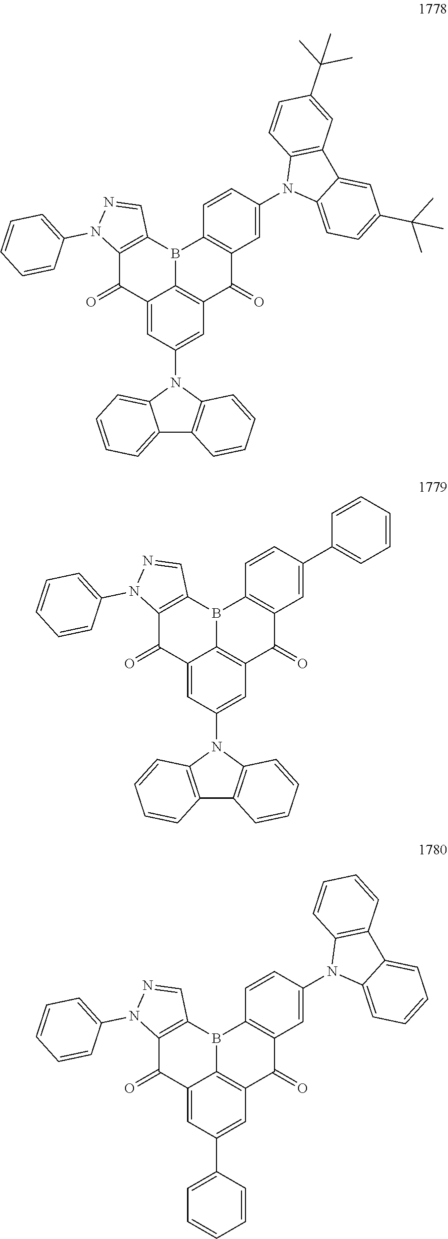 [SMILES: CC(C)(C)c1ccc2c(c1)c1cc(C(C)(C)C)ccc1n2-c1ccc2c(c1)C(=O)c1cc(-n3c4ccccc4c4ccccc43)cc3c1B2c1cnn(-c2ccccc2)c1C3=O ; O=C1c2cc(-c3ccccc3)ccc2B2c3cnn(-c4ccccc4)c3C(=O)c3cc(-n4c5ccccc5c5ccccc54)cc1c32 ; O=C1c2cc(-n3c4ccccc4c4ccccc43)ccc2B2c3cnn(-c4ccccc4)c3C(=O)c3cc(-c4ccccc4)cc1c32]

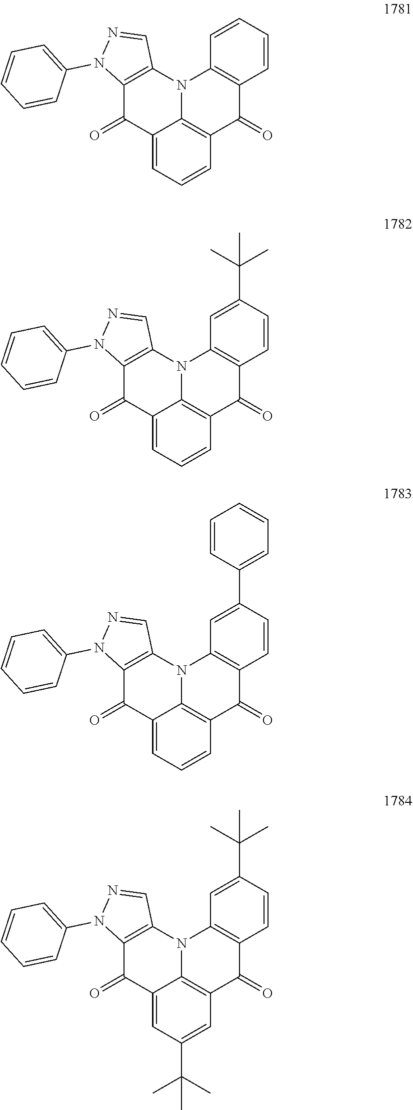 [SMILES: O=c1c2ccccc2n2c3cnn(-c4ccccc4)c3c(=O)c3cccc1c32 ; CC(C)(C)c1ccc2c(=O)c3cccc4c(=O)c5c(cnn5-c5ccccc5)n(c2c1)c34 ; O=c1c2ccc(-c3ccccc3)cc2n2c3cnn(-c4ccccc4)c3c(=O)c3cccc1c32 ; CC(C)(C)c1cc2c(=O)c3ccc(C(C)(C)C)cc3n3c4cnn(-c5ccccc5)c4c(=O)c(c1)c23]

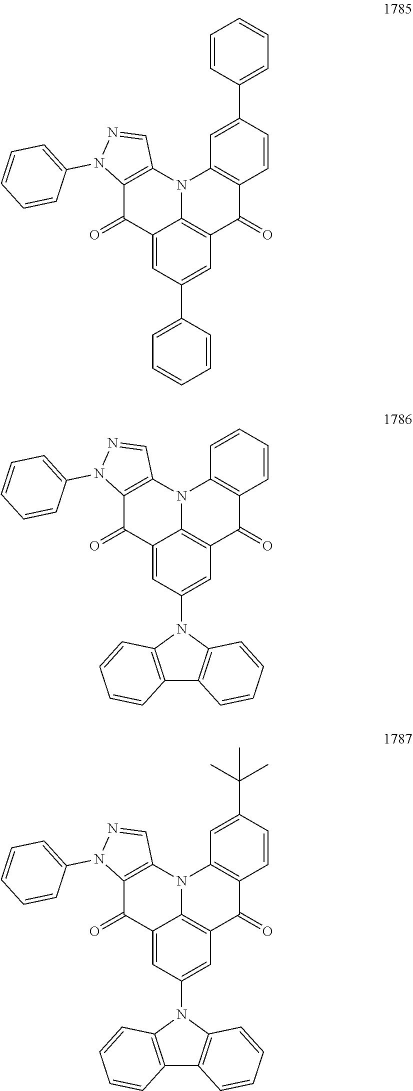

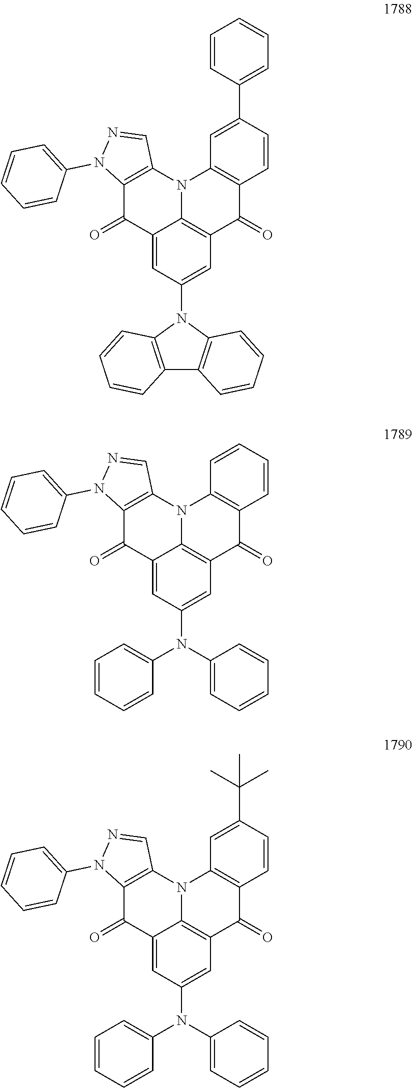

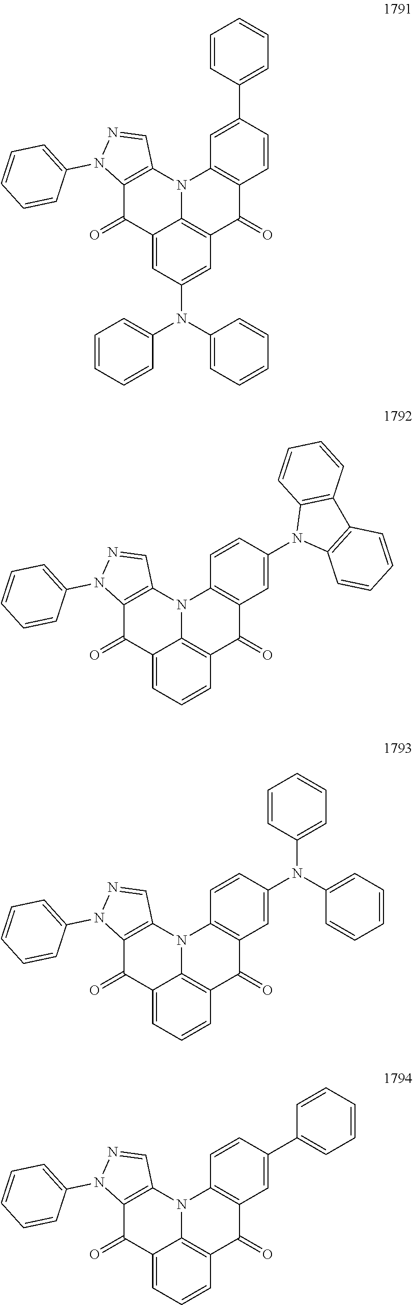 [SMILES: O=c1c2ccc(-c3ccccc3)cc2n2c3cnn(-c4ccccc4)c3c(=O)c3cc(N(c4ccccc4)c4ccccc4)cc1c32 ; O=c1c2cc(-n3c4ccccc4c4ccccc43)ccc2n2c3cnn(-c4ccccc4)c3c(=O)c3cccc1c32 ; O=c1c2cc(N(c3ccccc3)c3ccccc3)ccc2n2c3cnn(-c4ccccc4)c3c(=O)c3cccc1c32 ; O=c1c2cc(-c3ccccc3)ccc2n2c3cnn(-c4ccccc4)c3c(=O)c3cccc1c32]

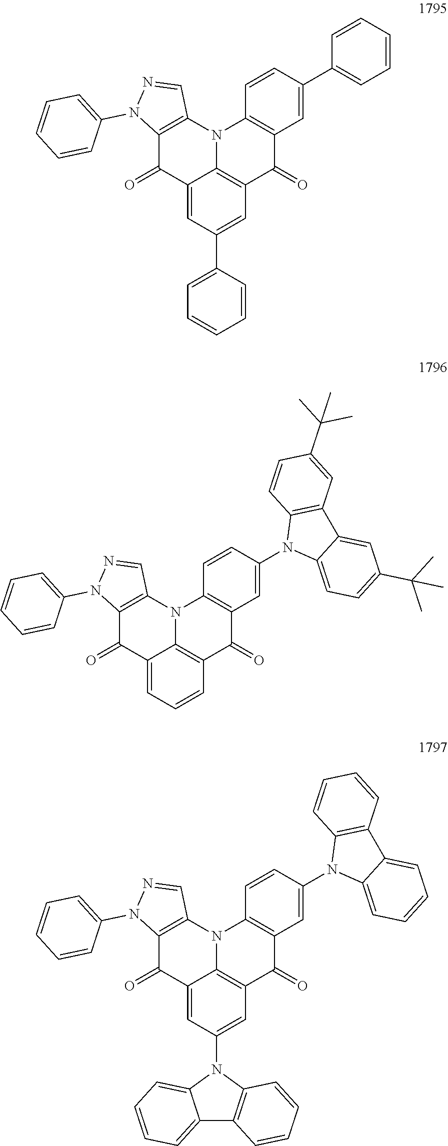 [SMILES: O=c1c2cc(-c3ccccc3)ccc2n2c3cnn(-c4ccccc4)c3c(=O)c3cc(-c4ccccc4)cc1c32 ; CC(C)(C)c1ccc2c(c1)c1cc(C(C)(C)C)ccc1n2-c1ccc2c(c1)c(=O)c1cccc3c(=O)c4c(cnn4-c4ccccc4)n2c13 ; O=c1c2cc(-n3c4ccccc4c4ccccc43)ccc2n2c3cnn(-c4ccccc4)c3c(=O)c3cc(-n4c5ccccc5c5ccccc54)cc1c32]

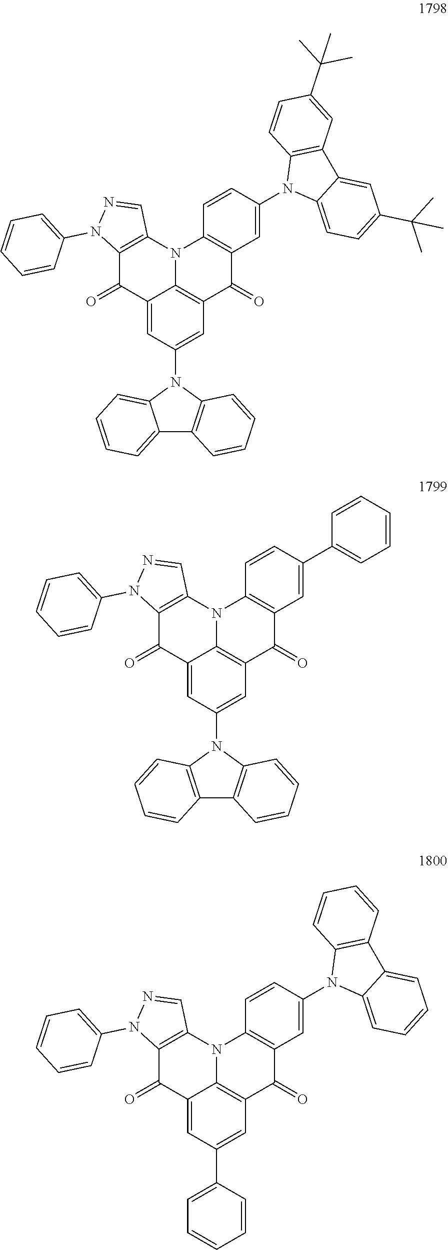 [SMILES: CC(C)(C)c1ccc2c(c1)c1cc(C(C)(C)C)ccc1n2-c1ccc2c(c1)c(=O)c1cc(-n3c4ccccc4c4ccccc43)cc3c(=O)c4c(cnn4-c4ccccc4)n2c13 ; O=c1c2cc(-c3ccccc3)ccc2n2c3cnn(-c4ccccc4)c3c(=O)c3cc(-n4c5ccccc5c5ccccc54)cc1c32 ; O=c1c2cc(-n3c4ccccc4c4ccccc43)ccc2n2c3cnn(-c4ccccc4)c3c(=O)c3cc(-c4ccccc4)cc1c32]

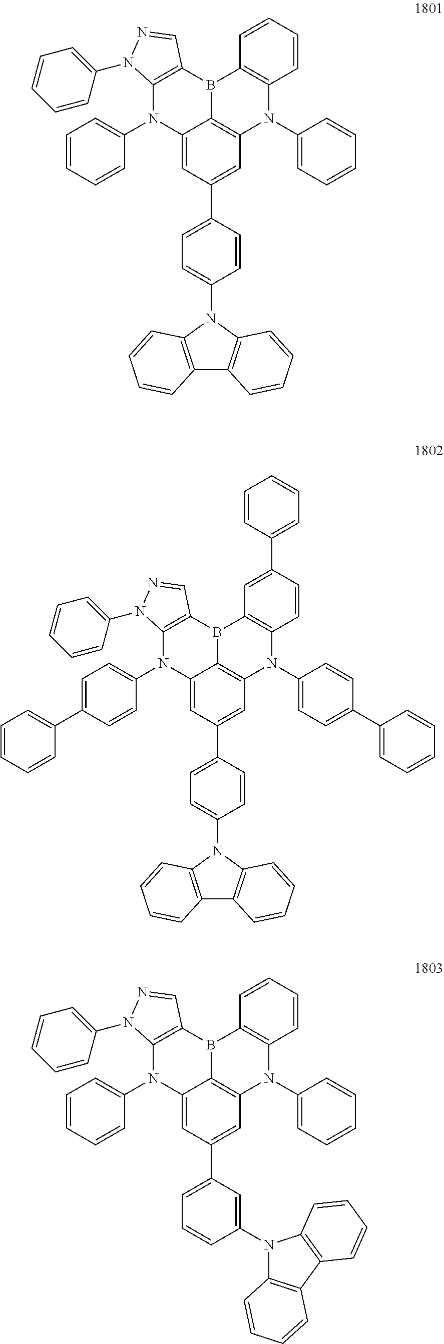 [SMILES: c1ccc(N2c3ccccc3B3c4cnn(-c5ccccc5)c4N(c4ccccc4)c4cc(-c5ccc(-n6c7ccccc7c7ccccc76)cc5)cc2c43)cc1 ; c1ccc(-c2ccc(N3c4ccc(-c5ccccc5)cc4B4c5cnn(-c6ccccc6)c5N(c5ccc(-c6ccccc6)cc5)c5cc(-c6ccc(-n7c8ccccc8c8ccccc87)cc6)cc3c54)cc2)cc1 ; c1ccc(N2c3ccccc3B3c4cnn(-c5ccccc5)c4N(c4ccccc4)c4cc(-c5cccc(-n6c7ccccc7c7ccccc76)c5)cc2c43)cc1]

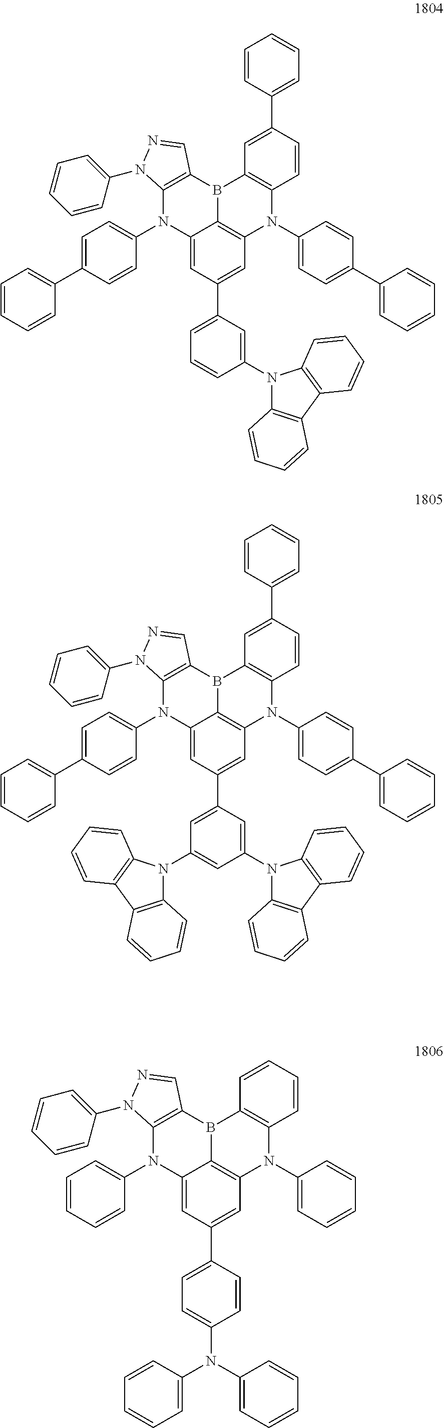 [SMILES: c1ccc(-c2ccc(N3c4ccc(-c5ccccc5)cc4B4c5cnn(-c6ccccc6)c5N(c5ccc(-c6ccccc6)cc5)c5cc(-c6cccc(-n7c8ccccc8c8ccccc87)c6)cc3c54)cc2)cc1 ; c1ccc(-c2ccc(N3c4ccc(-c5ccccc5)cc4B4c5cnn(-c6ccccc6)c5N(c5ccc(-c6ccccc6)cc5)c5cc(-c6cc(-n7c8ccccc8c8ccccc87)cc(-n7c8ccccc8c8ccccc87)c6)cc3c54)cc2)cc1 ; c1ccc(N(c2ccccc2)c2ccc(-c3cc4c5c(c3)N(c3ccccc3)c3c(cnn3-c3ccccc3)B5c3ccccc3N4c3ccccc3)cc2)cc1]

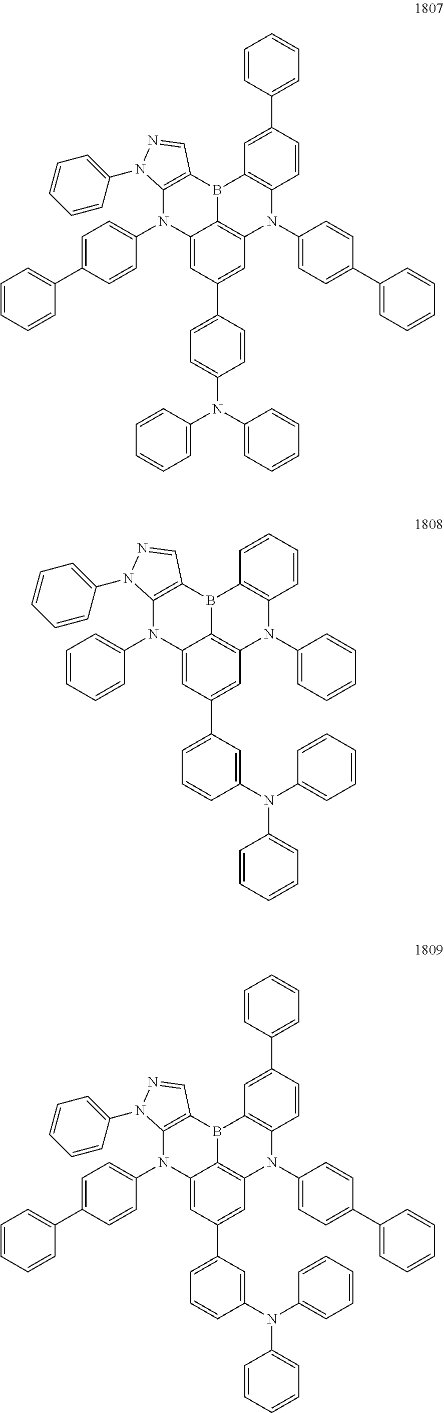 [SMILES: c1ccc(-c2ccc(N3c4ccc(-c5ccccc5)cc4B4c5cnn(-c6ccccc6)c5N(c5ccc(-c6ccccc6)cc5)c5cc(-c6ccc(N(c7ccccc7)c7ccccc7)cc6)cc3c54)cc2)cc1 ; c1ccc(N(c2ccccc2)c2cccc(-c3cc4c5c(c3)N(c3ccccc3)c3c(cnn3-c3ccccc3)B5c3ccccc3N4c3ccccc3)c2)cc1 ; c1ccc(-c2ccc(N3c4ccc(-c5ccccc5)cc4B4c5cnn(-c6ccccc6)c5N(c5ccc(-c6ccccc6)cc5)c5cc(-c6cccc(N(c7ccccc7)c7ccccc7)c6)cc3c54)cc2)cc1]

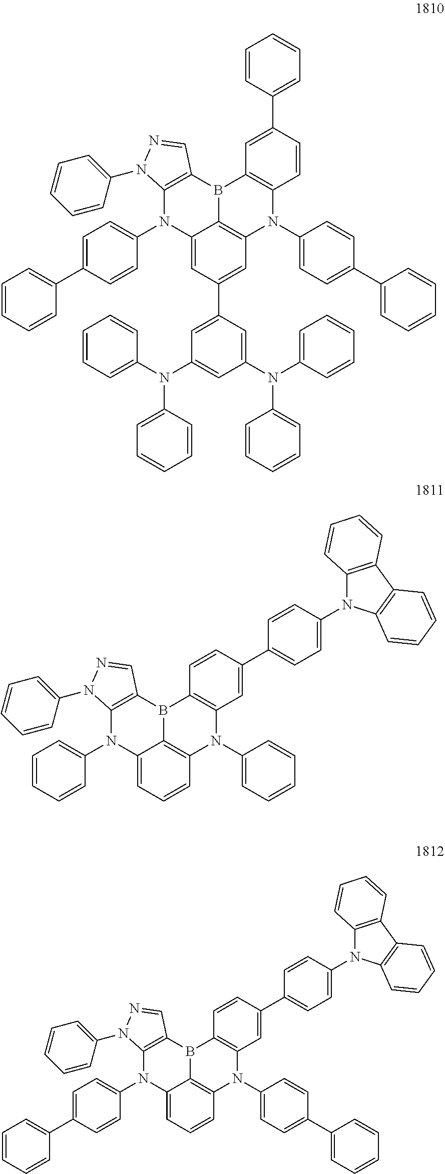 [SMILES: c1ccc(-c2ccc(N3c4ccc(-c5ccccc5)cc4B4c5cnn(-c6ccccc6)c5N(c5ccc(-c6ccccc6)cc5)c5cc(-c6cc(N(c7ccccc7)c7ccccc7)cc(N(c7ccccc7)c7ccccc7)c6)cc3c54)cc2)cc1 ; c1ccc(N2c3cc(-c4ccc(-n5c6ccccc6c6ccccc65)cc4)ccc3B3c4cnn(-c5ccccc5)c4N(c4ccccc4)c4cccc2c43)cc1 ; c1ccc(-c2ccc(N3c4cc(-c5ccc(-n6c7ccccc7c7ccccc76)cc5)ccc4B4c5cnn(-c6ccccc6)c5N(c5ccc(-c6ccccc6)cc5)c5cccc3c54)cc2)cc1]

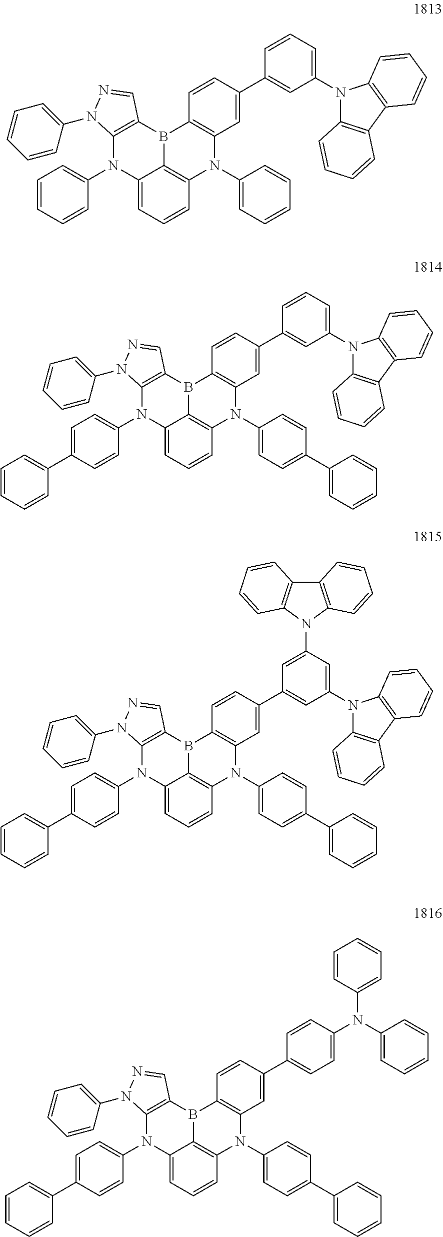 [SMILES: c1ccc(N2c3cc(-c4cccc(-n5c6ccccc6c6ccccc65)c4)ccc3B3c4cnn(-c5ccccc5)c4N(c4ccccc4)c4cccc2c43)cc1 ; c1ccc(-c2ccc(N3c4cc(-c5cccc(-n6c7ccccc7c7ccccc76)c5)ccc4B4c5cnn(-c6ccccc6)c5N(c5ccc(-c6ccccc6)cc5)c5cccc3c54)cc2)cc1 ; c1ccc(-c2ccc(N3c4cc(-c5cc(-n6c7ccccc7c7ccccc76)cc(-n6c7ccccc7c7ccccc76)c5)ccc4B4c5cnn(-c6ccccc6)c5N(c5ccc(-c6ccccc6)cc5)c5cccc3c54)cc2)cc1 ; c1ccc(-c2ccc(N3c4cc(-c5ccc(N(c6ccccc6)c6ccccc6)cc5)ccc4B4c5cnn(-c6ccccc6)c5N(c5ccc(-c6ccccc6)cc5)c5cccc3c54)cc2)cc1]

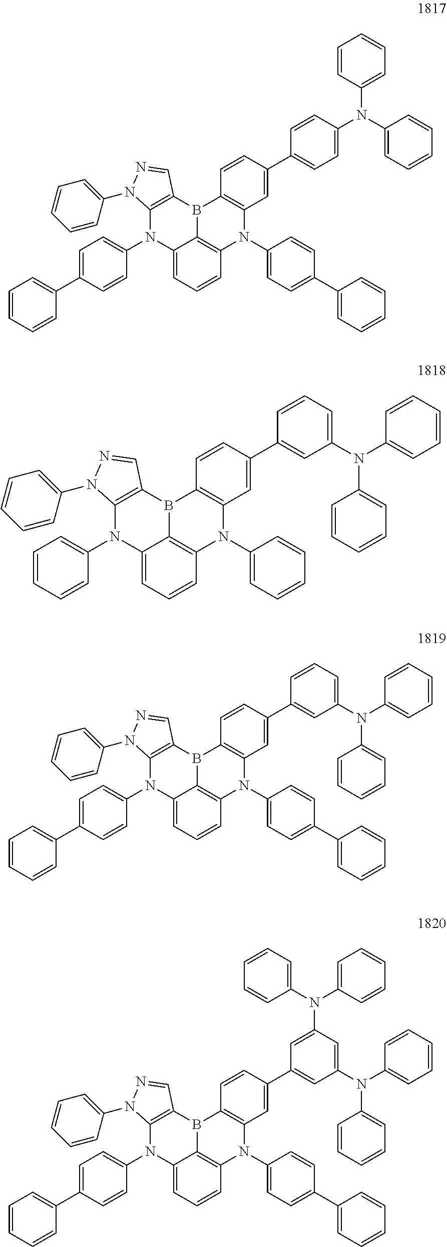 [SMILES: c1ccc(-c2ccc(N3c4cc(-c5ccc(N(c6ccccc6)c6ccccc6)cc5)ccc4B4c5cnn(-c6ccccc6)c5N(c5ccc(-c6ccccc6)cc5)c5cccc3c54)cc2)cc1 ; c1ccc(N(c2ccccc2)c2cccc(-c3ccc4c(c3)N(c3ccccc3)c3cccc5c3B4c3cnn(-c4ccccc4)c3N5c3ccccc3)c2)cc1 ; c1ccc(-c2ccc(N3c4cc(-c5cccc(N(c6ccccc6)c6ccccc6)c5)ccc4B4c5cnn(-c6ccccc6)c5N(c5ccc(-c6ccccc6)cc5)c5cccc3c54)cc2)cc1 ; c1ccc(-c2ccc(N3c4cc(-c5cc(N(c6ccccc6)c6ccccc6)cc(N(c6ccccc6)c6ccccc6)c5)ccc4B4c5cnn(-c6ccccc6)c5N(c5ccc(-c6ccccc6)cc5)c5cccc3c54)cc2)cc1]

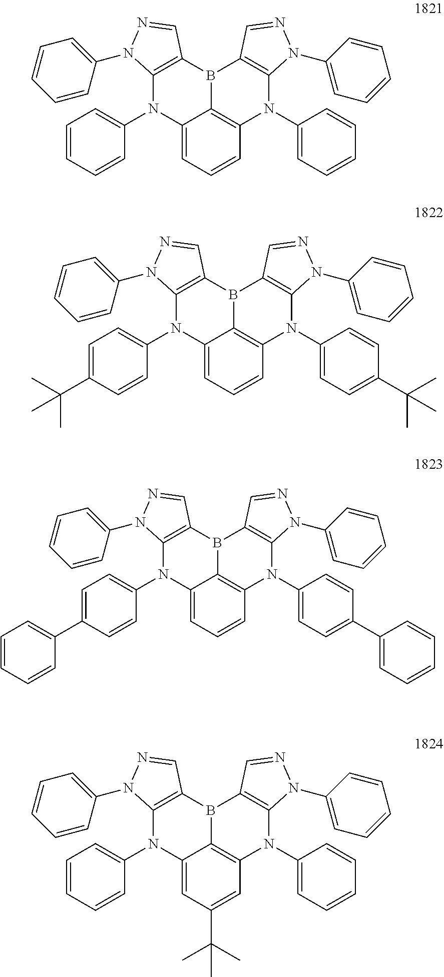 [SMILES: c1ccc(N2c3cccc4c3B(c3cnn(-c5ccccc5)c32)c2cnn(-c3ccccc3)c2N4c2ccccc2)cc1 ; CC(C)(C)c1ccc(N2c3cccc4c3B(c3cnn(-c5ccccc5)c32)c2cnn(-c3ccccc3)c2N4c2ccc(C(C)(C)C)cc2)cc1 ; c1ccc(-c2ccc(N3c4cccc5c4B(c4cnn(-c6ccccc6)c43)c3cnn(-c4ccccc4)c3N5c3ccc(-c4ccccc4)cc3)cc2)cc1 ; CC(C)(C)c1cc2c3c(c1)N(c1ccccc1)c1c(cnn1-c1ccccc1)B3c1cnn(-c3ccccc3)c1N2c1ccccc1]

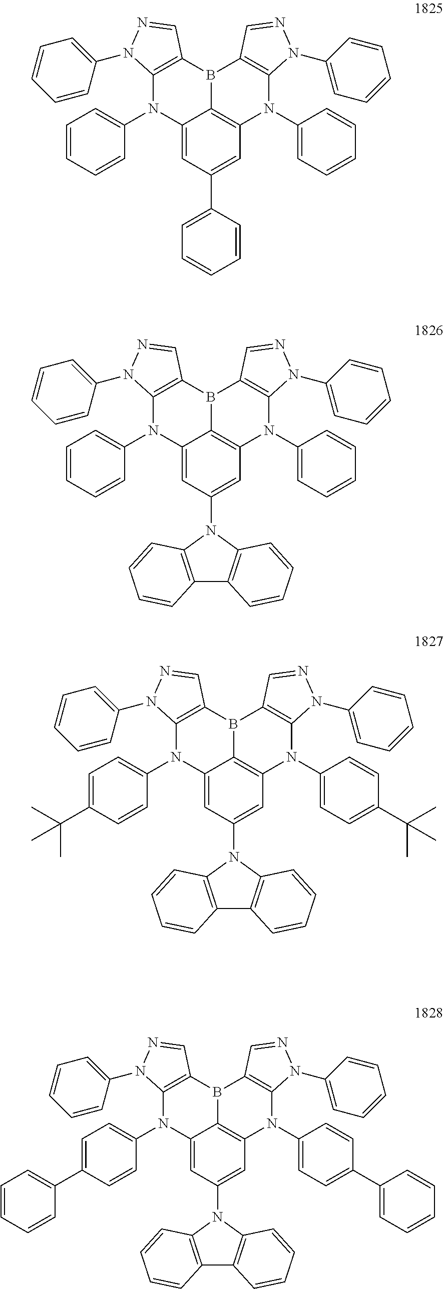 [SMILES: c1ccc(-c2cc3c4c(c2)N(c2ccccc2)c2c(cnn2-c2ccccc2)B4c2cnn(-c4ccccc4)c2N3c2ccccc2)cc1 ; c1ccc(N2c3cc(-n4c5ccccc5c5ccccc54)cc4c3B(c3cnn(-c5ccccc5)c32)c2cnn(-c3ccccc3)c2N4c2ccccc2)cc1 ; CC(C)(C)c1ccc(N2c3cc(-n4c5ccccc5c5ccccc54)cc4c3B(c3cnn(-c5ccccc5)c32)c2cnn(-c3ccccc3)c2N4c2ccc(C(C)(C)C)cc2)cc1 ; c1ccc(-c2ccc(N3c4cc(-n5c6ccccc6c6ccccc65)cc5c4B(c4cnn(-c6ccccc6)c43)c3cnn(-c4ccccc4)c3N5c3ccc(-c4ccccc4)cc3)cc2)cc1]

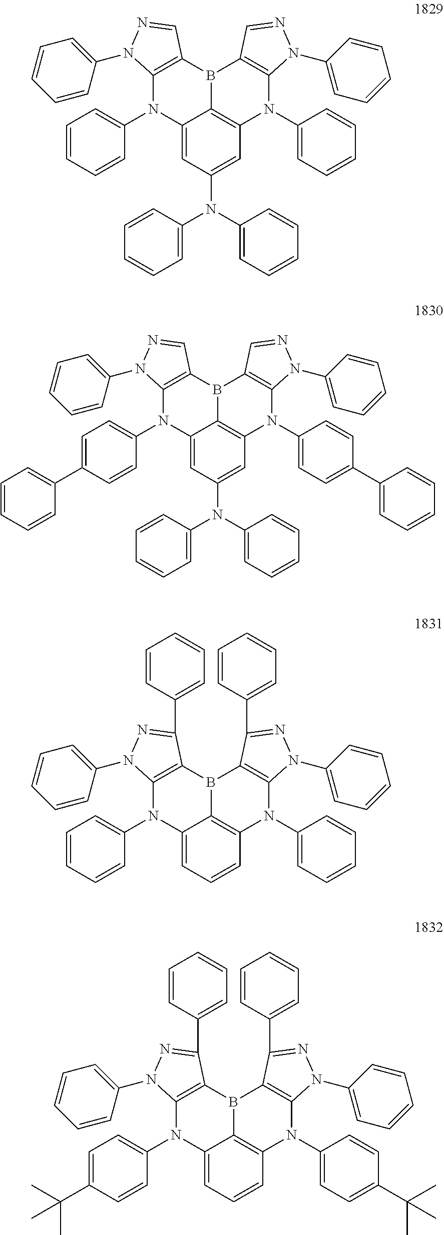 [SMILES: c1ccc(N(c2ccccc2)c2cc3c4c(c2)N(c2ccccc2)c2c(cnn2-c2ccccc2)B4c2cnn(-c4ccccc4)c2N3c2ccccc2)cc1 ; c1ccc(-c2ccc(N3c4cc(N(c5ccccc5)c5ccccc5)cc5c4B(c4cnn(-c6ccccc6)c43)c3cnn(-c4ccccc4)c3N5c3ccc(-c4ccccc4)cc3)cc2)cc1 ; c1ccc(-c2nn(-c3ccccc3)c3c2B2c4c(cccc4N(c4ccccc4)c4c2c(-c2ccccc2)nn4-c2ccccc2)N3c2ccccc2)cc1 ; CC(C)(C)c1ccc(N2c3cccc4c3B(c3c(-c5ccccc5)nn(-c5ccccc5)c32)c2c(-c3ccccc3)nn(-c3ccccc3)c2N4c2ccc(C(C)(C)C)cc2)cc1]

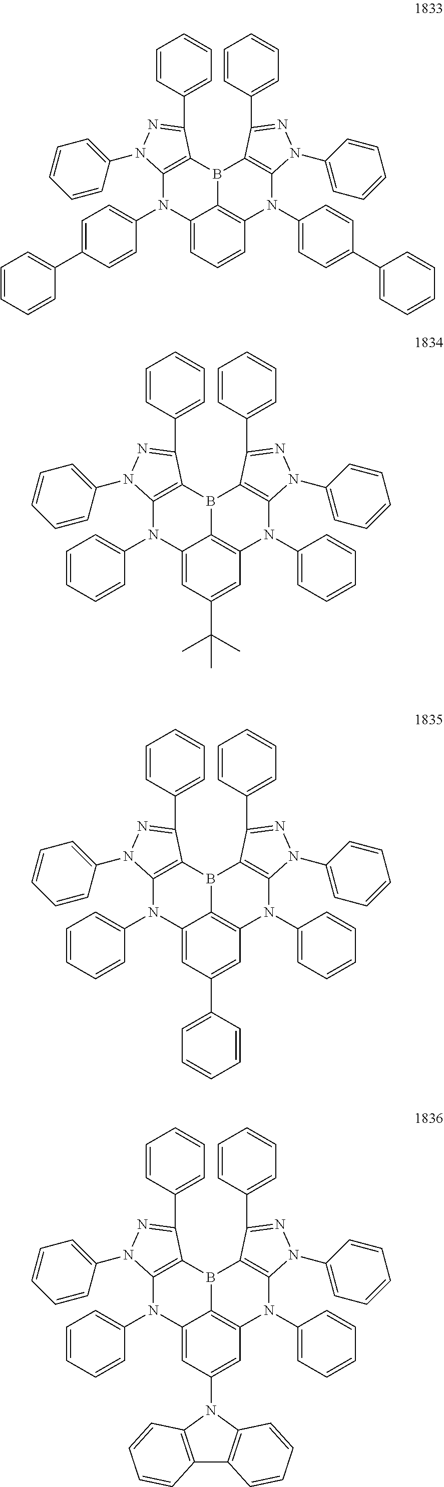 [SMILES: c1ccc(-c2ccc(N3c4cccc5c4B(c4c(-c6ccccc6)nn(-c6ccccc6)c43)c3c(-c4ccccc4)nn(-c4ccccc4)c3N5c3ccc(-c4ccccc4)cc3)cc2)cc1 ; CC(C)(C)c1cc2c3c(c1)N(c1ccccc1)c1c(c(-c4ccccc4)nn1-c1ccccc1)B3c1c(-c3ccccc3)nn(-c3ccccc3)c1N2c1ccccc1 ; c1ccc(-c2cc3c4c(c2)N(c2ccccc2)c2c(c(-c5ccccc5)nn2-c2ccccc2)B4c2c(-c4ccccc4)nn(-c4ccccc4)c2N3c2ccccc2)cc1 ; c1ccc(-c2nn(-c3ccccc3)c3c2B2c4c(cc(-n5c6ccccc6c6ccccc65)cc4N(c4ccccc4)c4c2c(-c2ccccc2)nn4-c2ccccc2)N3c2ccccc2)cc1]

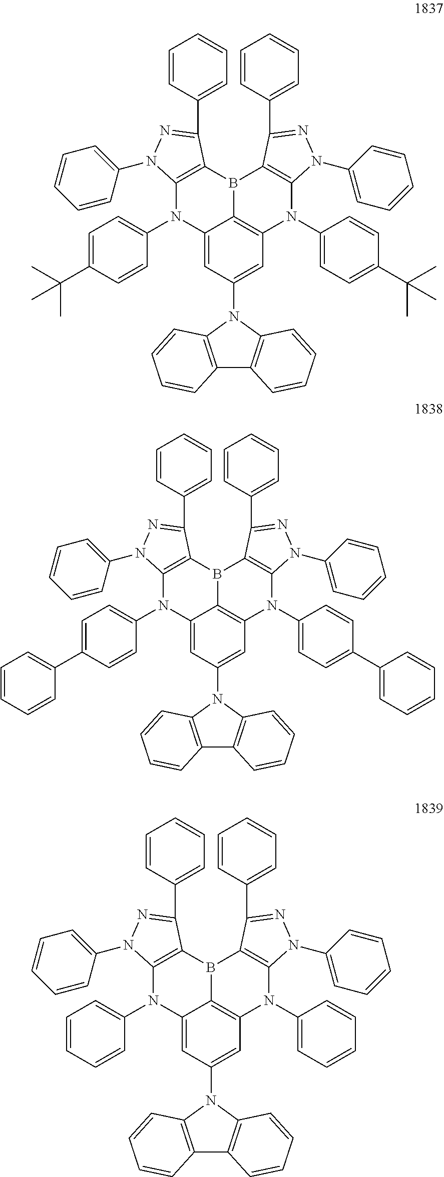

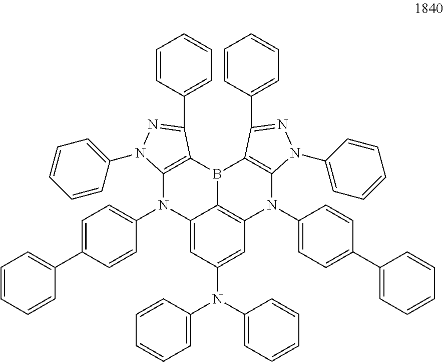 [SMILES: c1ccc(-c2ccc(N3c4cc(N(c5ccccc5)c5ccccc5)cc5c4B(c4c(-c6ccccc6)nn(-c6ccccc6)c43)c3c(-c4ccccc4)nn(-c4ccccc4)c3N5c3ccc(-c4ccccc4)cc3)cc2)cc1]

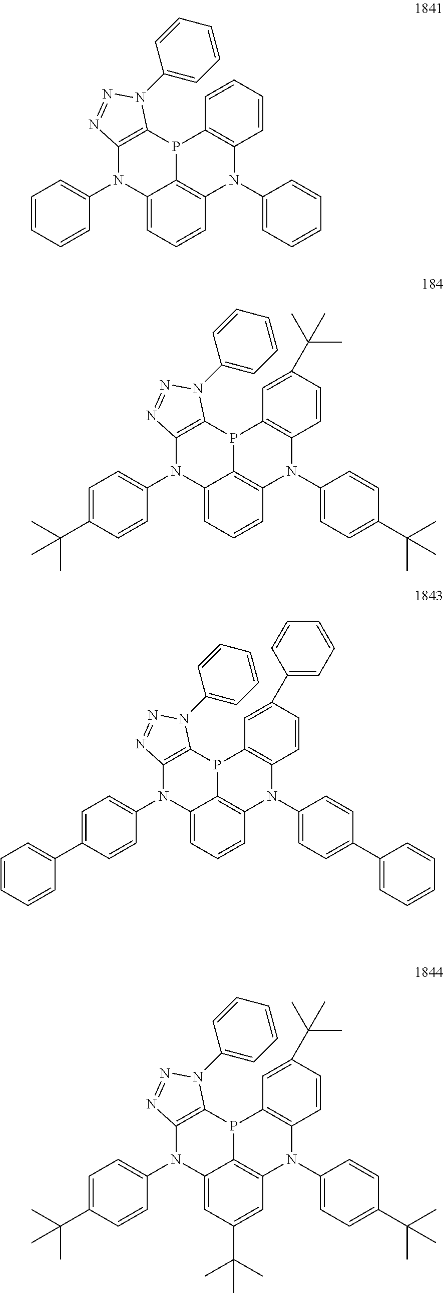

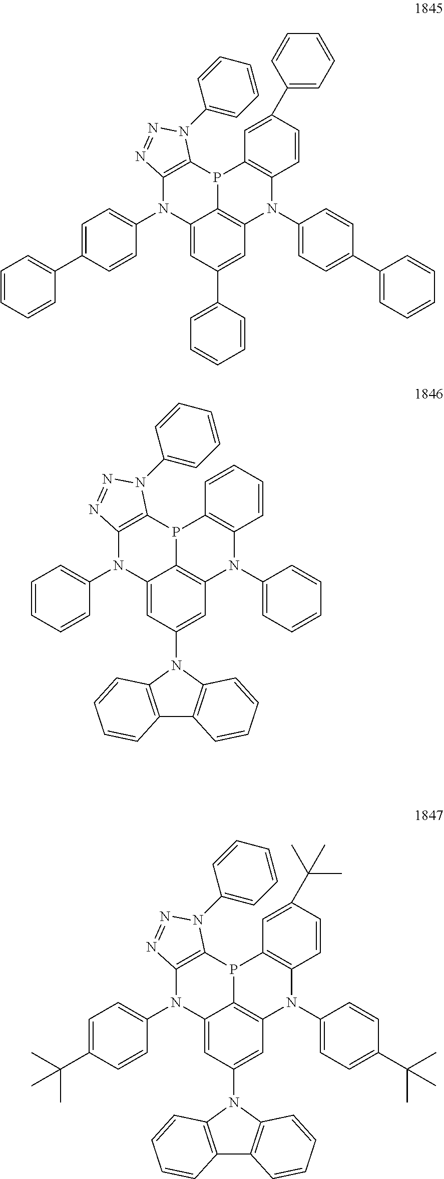 [SMILES: c1ccc(-c2ccc(N3c4ccc(-c5ccccc5)cc4P4c5c3cc(-c3ccccc3)cc5N(c3ccc(-c5ccccc5)cc3)c3nnn(-c5ccccc5)c34)cc2)cc1 ; c1ccc(N2c3ccccc3P3c4c2cc(-n2c5ccccc5c5ccccc52)cc4N(c2ccccc2)c2nnn(-c4ccccc4)c23)cc1 ; CC(C)(C)c1ccc(N2c3ccc(C(C)(C)C)cc3P3c4c2cc(-n2c5ccccc5c5ccccc52)cc4N(c2ccc(C(C)(C)C)cc2)c2nnn(-c4ccccc4)c23)cc1]

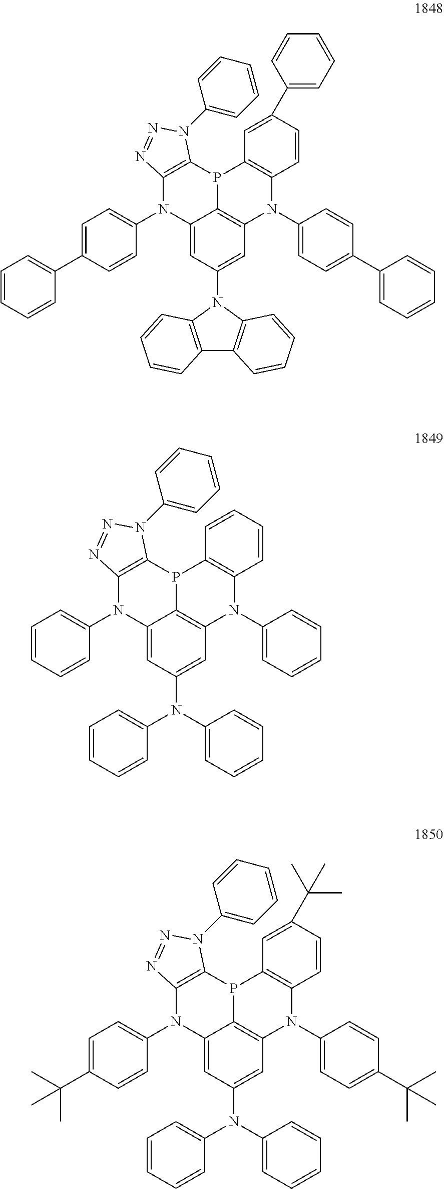 [SMILES: c1ccc(-c2ccc(N3c4ccc(-c5ccccc5)cc4P4c5c3cc(-n3c6ccccc6c6ccccc63)cc5N(c3ccc(-c5ccccc5)cc3)c3nnn(-c5ccccc5)c34)cc2)cc1 ; c1ccc(N(c2ccccc2)c2cc3c4c(c2)N(c2ccccc2)c2nnn(-c5ccccc5)c2P4c2ccccc2N3c2ccccc2)cc1 ; CC(C)(C)c1ccc(N2c3ccc(C(C)(C)C)cc3P3c4c2cc(N(c2ccccc2)c2ccccc2)cc4N(c2ccc(C(C)(C)C)cc2)c2nnn(-c4ccccc4)c23)cc1]

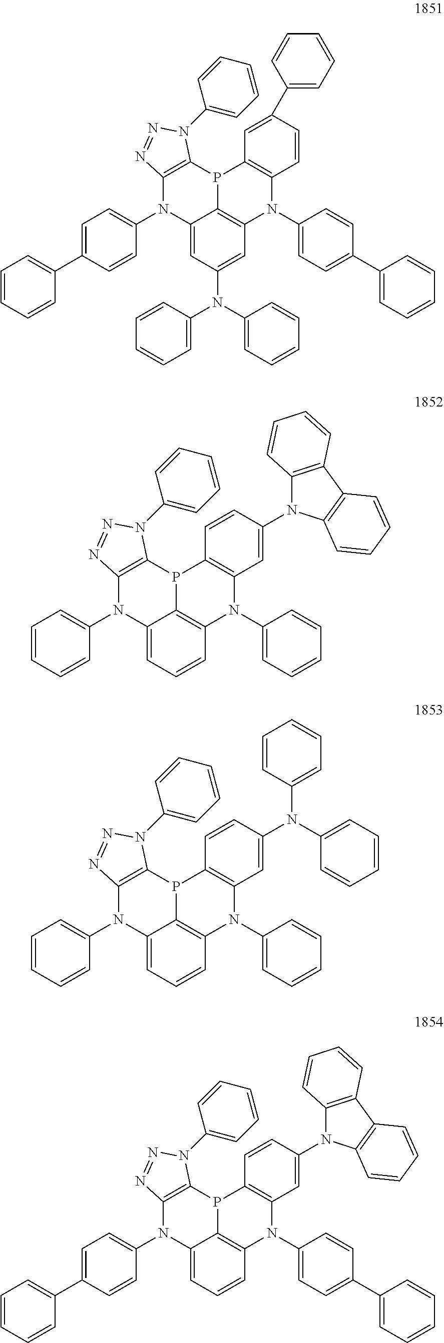

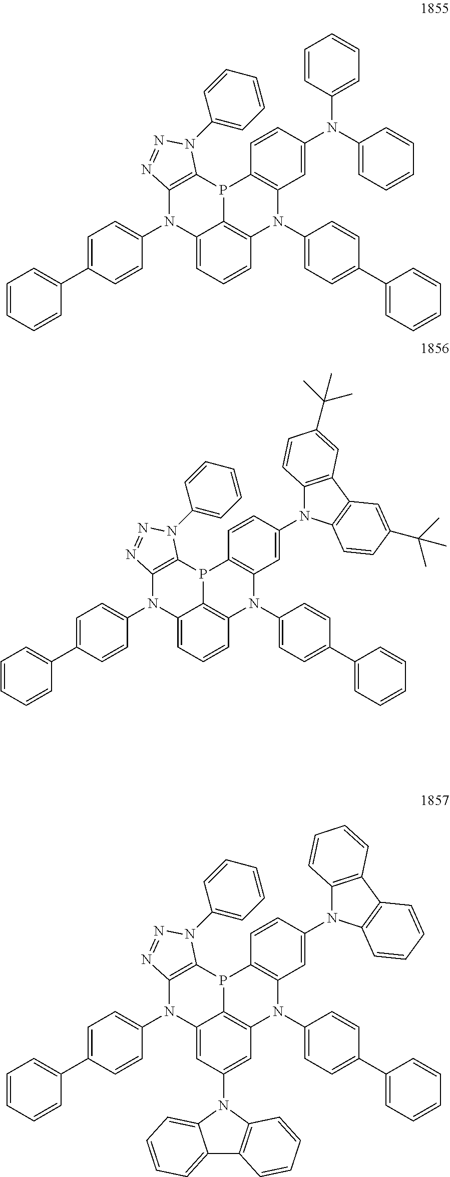 [SMILES: c1ccc(-c2ccc(N3c4cc(N(c5ccccc5)c5ccccc5)ccc4P4c5c3cccc5N(c3ccc(-c5ccccc5)cc3)c3nnn(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1P2c1c(nnn1-c1ccccc1)N3c1ccc(-c2ccccc2)cc1 ; c1ccc(-c2ccc(N3c4cc(-n5c6ccccc6c6ccccc65)ccc4P4c5c3cc(-n3c6ccccc6c6ccccc63)cc5N(c3ccc(-c5ccccc5)cc3)c3nnn(-c5ccccc5)c34)cc2)cc1]

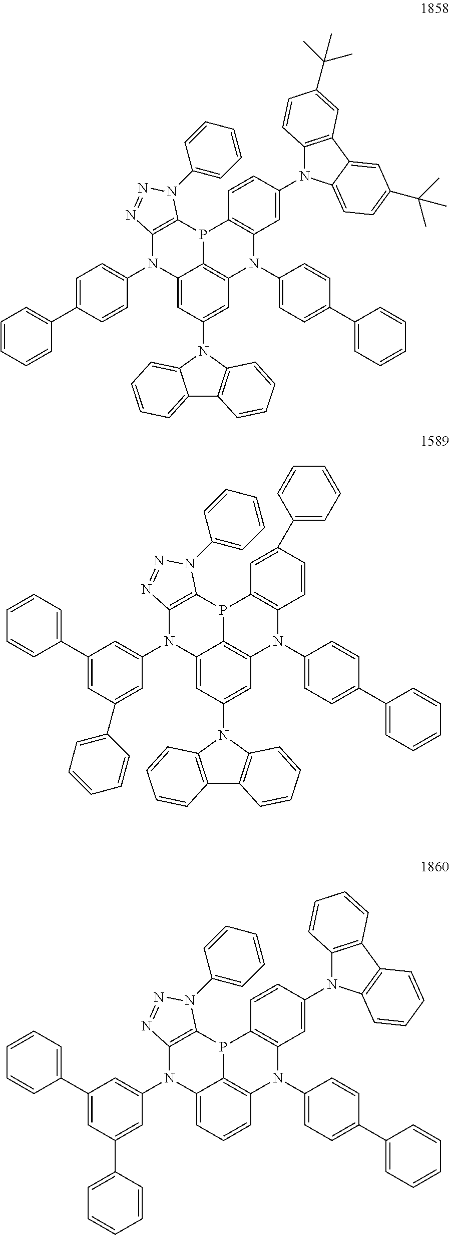 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1P2c1c(nnn1-c1ccccc1)N3c1ccc(-c2ccccc2)cc1 ; c1ccc(-c2ccc(N3c4ccc(-c5ccccc5)cc4P4c5c3cc(-n3c6ccccc6c6ccccc63)cc5N(c3cc(-c5ccccc5)cc(-c5ccccc5)c3)c3nnn(-c5ccccc5)c34)cc2)cc1 ; c1ccc(-c2ccc(N3c4cc(-n5c6ccccc6c6ccccc65)ccc4P4c5c3cccc5N(c3cc(-c5ccccc5)cc(-c5ccccc5)c3)c3nnn(-c5ccccc5)c34)cc2)cc1]

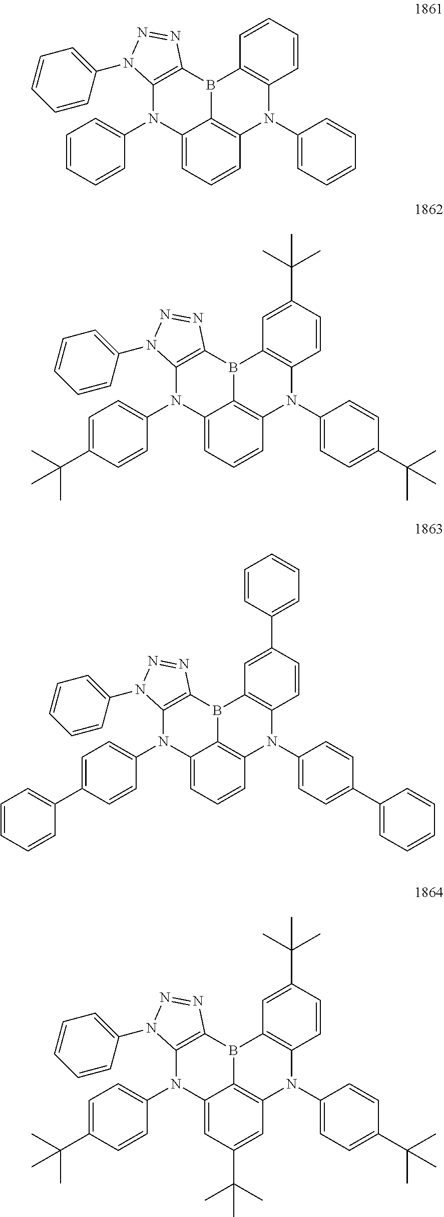

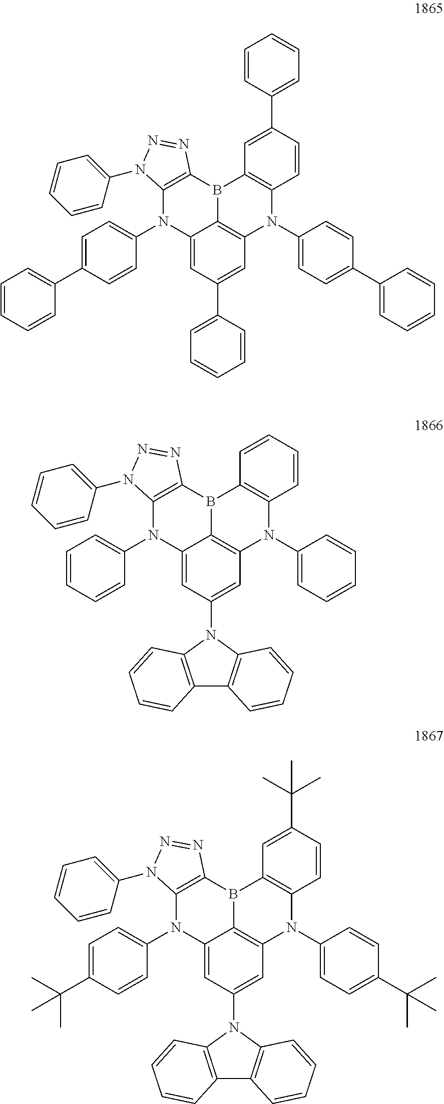 [SMILES: c1ccc(-c2ccc(N3c4ccc(-c5ccccc5)cc4B4c5nnn(-c6ccccc6)c5N(c5ccc(-c6ccccc6)cc5)c5cc(-c6ccccc6)cc3c54)cc2)cc1 ; c1ccc(N2c3ccccc3B3c4nnn(-c5ccccc5)c4N(c4ccccc4)c4cc(-n5c6ccccc6c6ccccc65)cc2c43)cc1 ; CC(C)(C)c1ccc(N2c3ccc(C(C)(C)C)cc3B3c4nnn(-c5ccccc5)c4N(c4ccc(C(C)(C)C)cc4)c4cc(-n5c6ccccc6c6ccccc65)cc2c43)cc1]

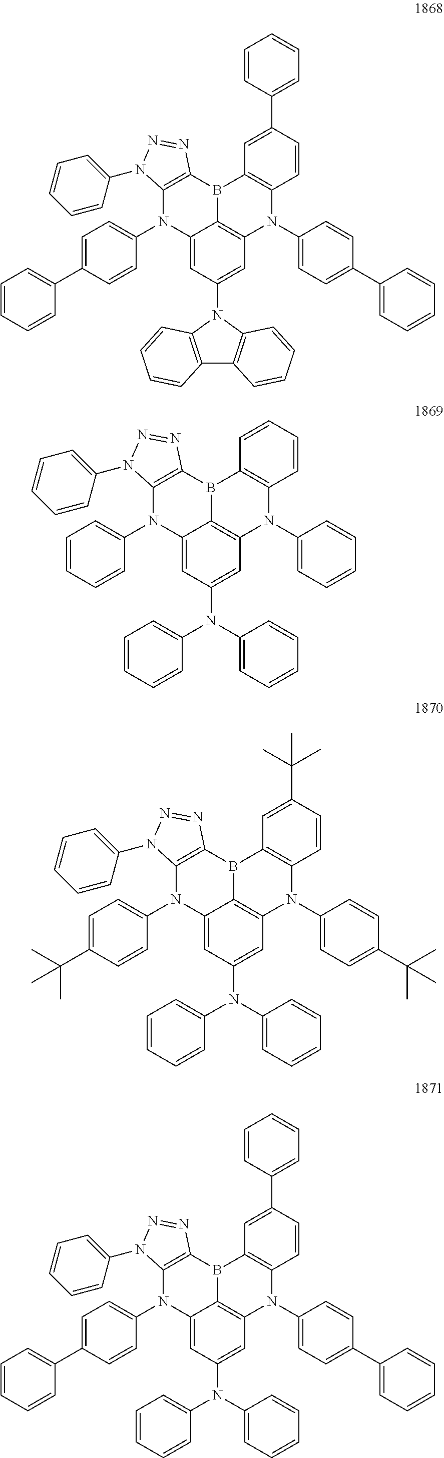

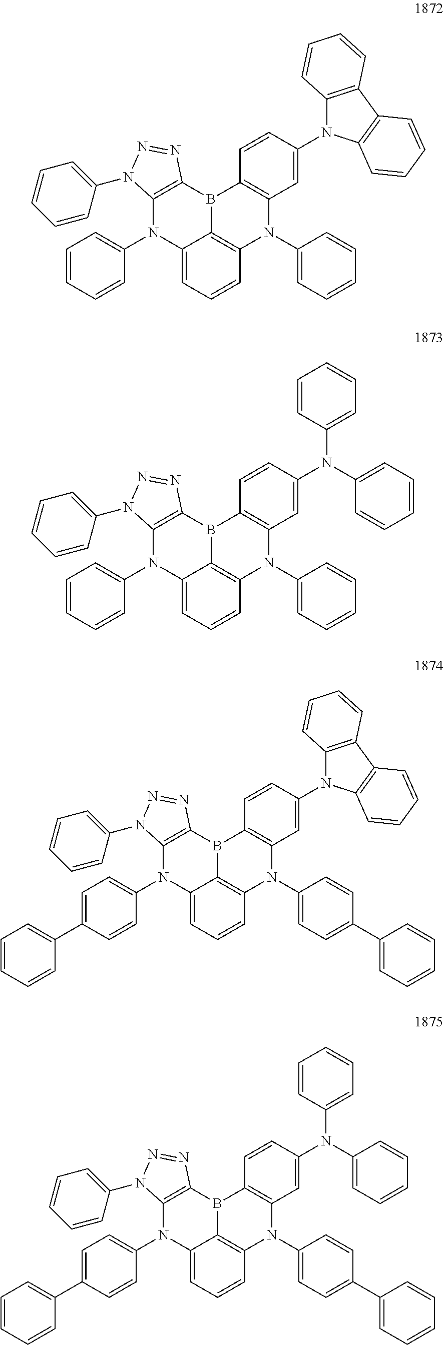 [SMILES: c1ccc(N2c3cc(-n4c5ccccc5c5ccccc54)ccc3B3c4nnn(-c5ccccc5)c4N(c4ccccc4)c4cccc2c43)cc1 ; c1ccc(N(c2ccccc2)c2ccc3c(c2)N(c2ccccc2)c2cccc4c2B3c2nnn(-c3ccccc3)c2N4c2ccccc2)cc1 ; c1ccc(-c2ccc(N3c4cc(-n5c6ccccc6c6ccccc65)ccc4B4c5nnn(-c6ccccc6)c5N(c5ccc(-c6ccccc6)cc5)c5cccc3c54)cc2)cc1 ; c1ccc(-c2ccc(N3c4cc(N(c5ccccc5)c5ccccc5)ccc4B4c5nnn(-c6ccccc6)c5N(c5ccc(-c6ccccc6)cc5)c5cccc3c54)cc2)cc1]

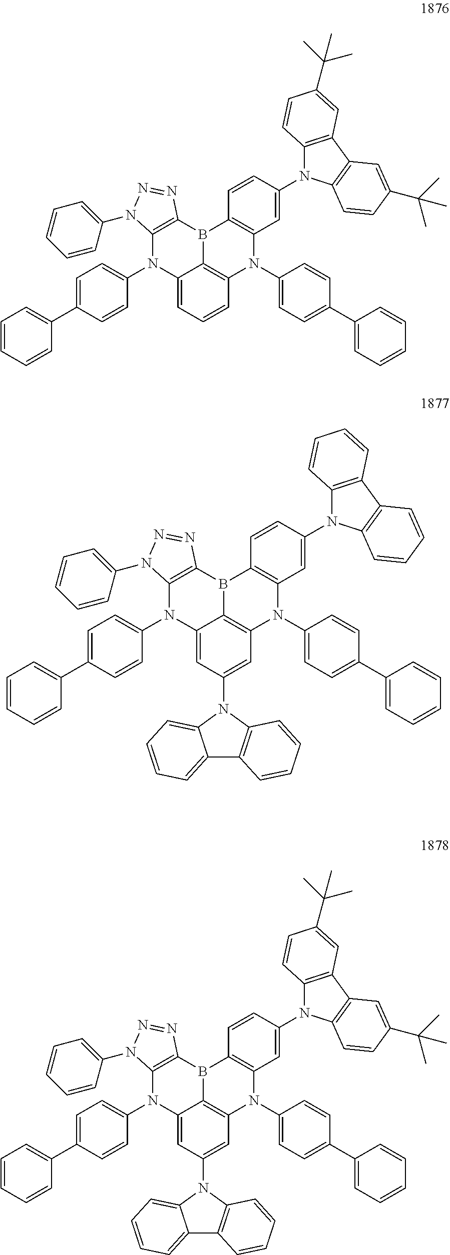 [SMILES: CC(C)(C)c1ccc2c(c1)c1cc(C(C)(C)C)ccc1n2-c1ccc2c(c1)N(c1ccc(-c3ccccc3)cc1)c1cccc3c1B2c1nnn(-c2ccccc2)c1N3c1ccc(-c2ccccc2)cc1 ; c1ccc(-c2ccc(N3c4cc(-n5c6ccccc6c6ccccc65)ccc4B4c5nnn(-c6ccccc6)c5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nnn(-c2ccccc2)c1N3c1ccc(-c2ccccc2)cc1]

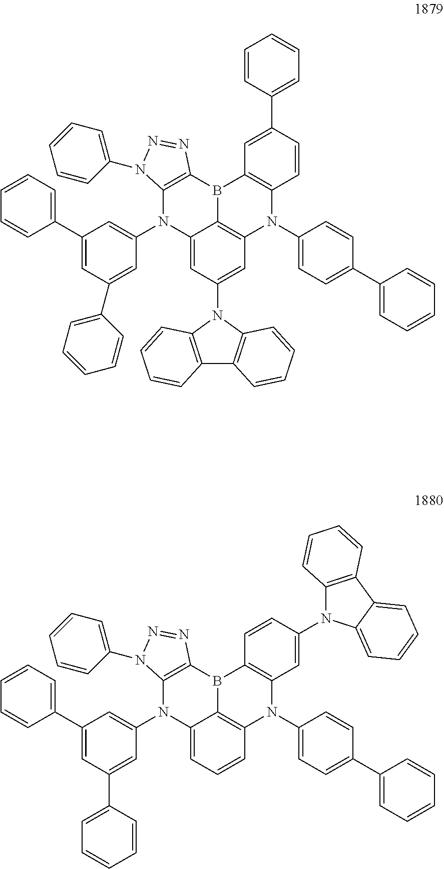 [SMILES: c1ccc(-c2ccc(N3c4ccc(-c5ccccc5)cc4B4c5nnn(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nnn(-c6ccccc6)c5N(c5cc(-c6ccccc6)cc(-c6ccccc6)c5)c5cccc3c54)cc2)cc1]

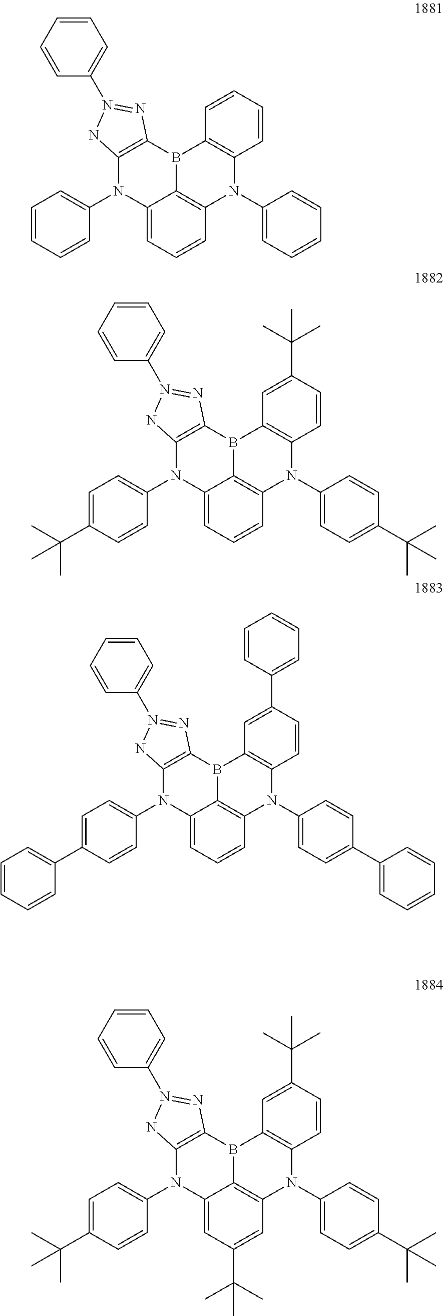

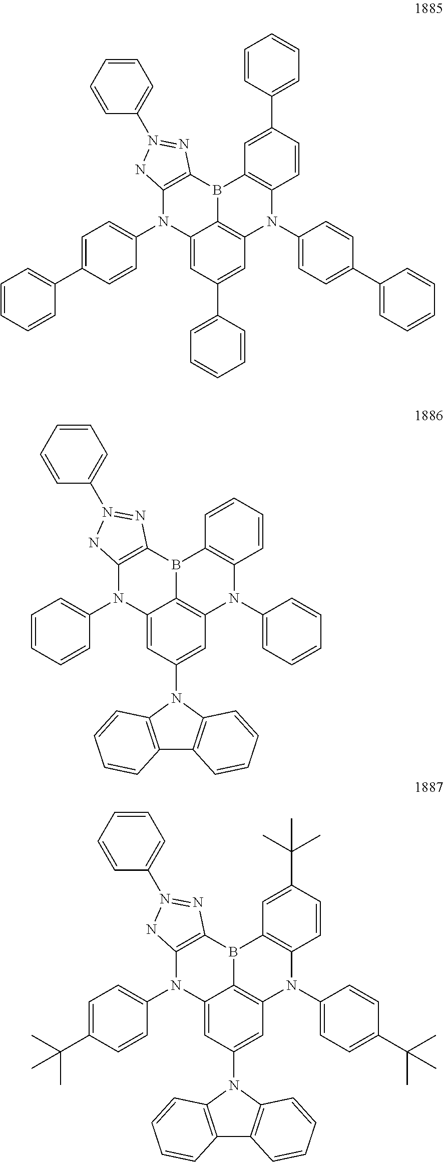

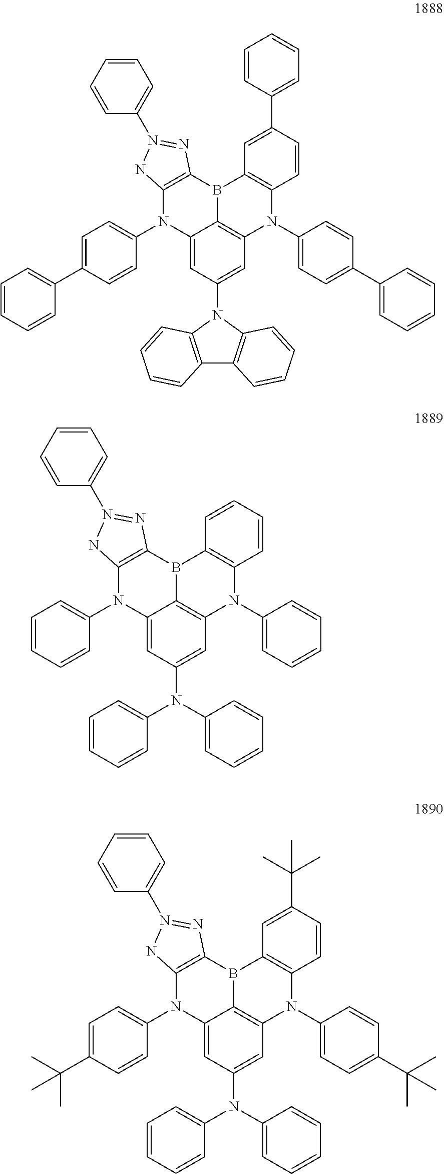

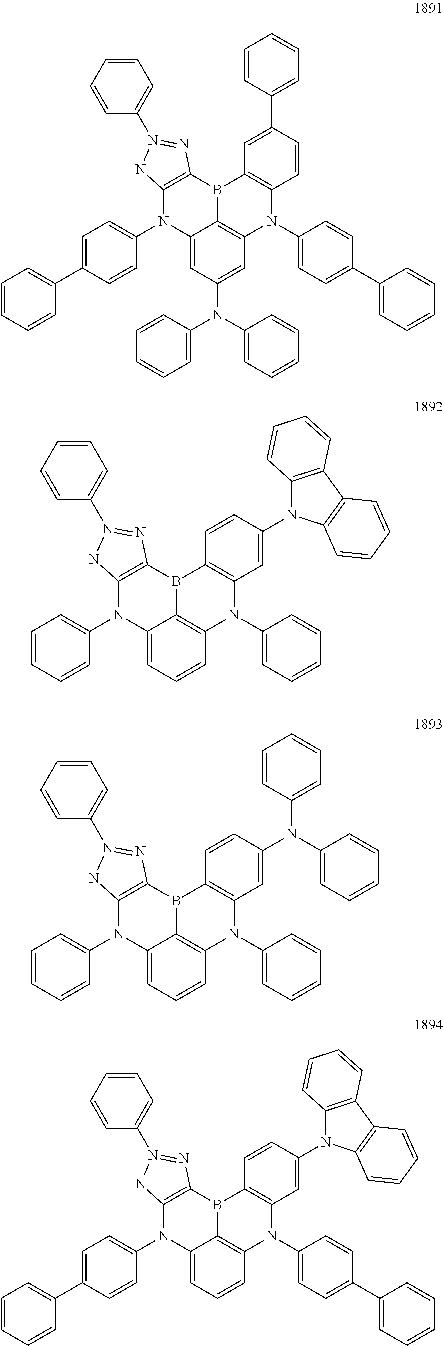

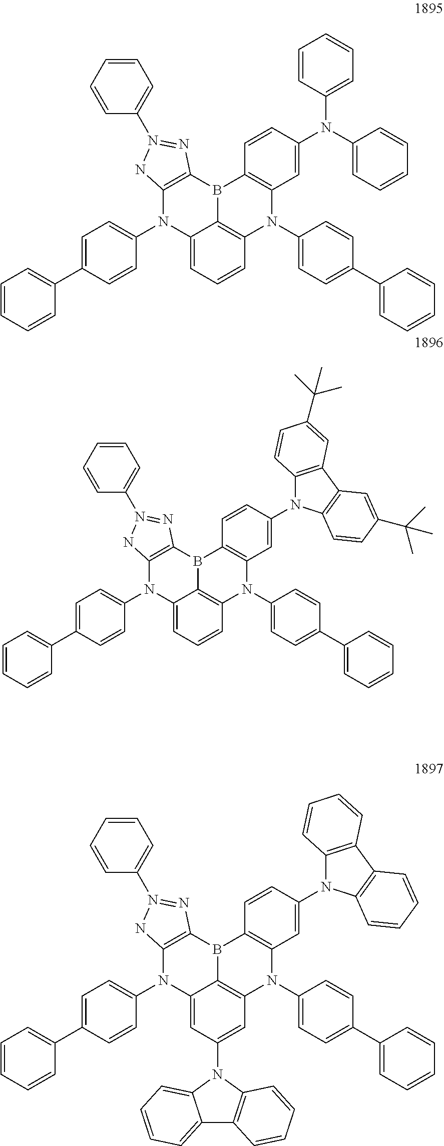

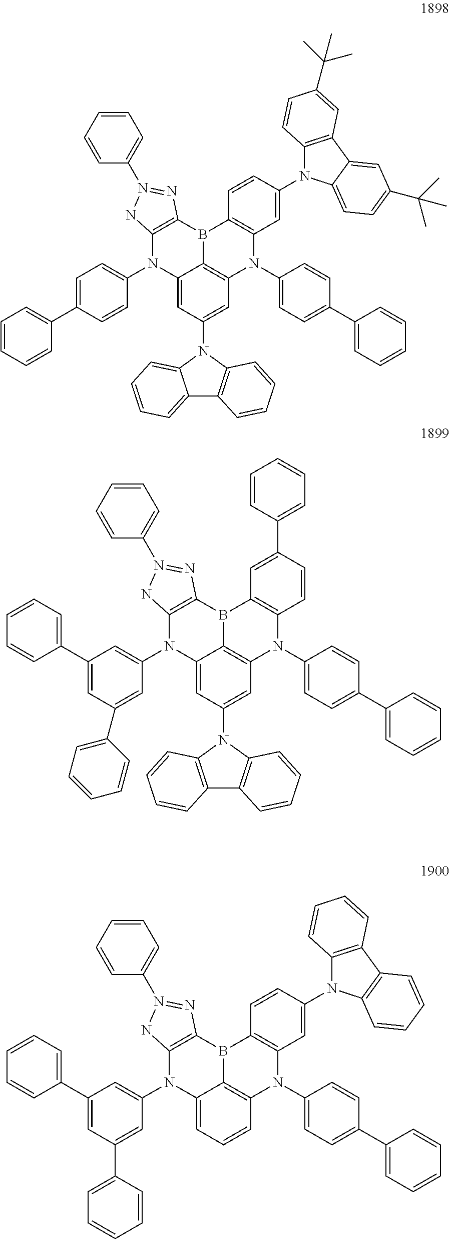

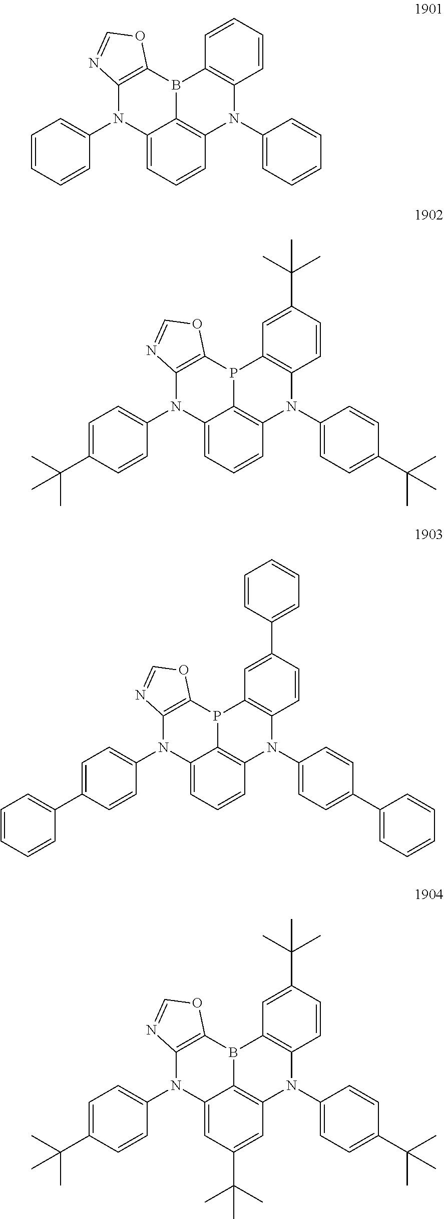

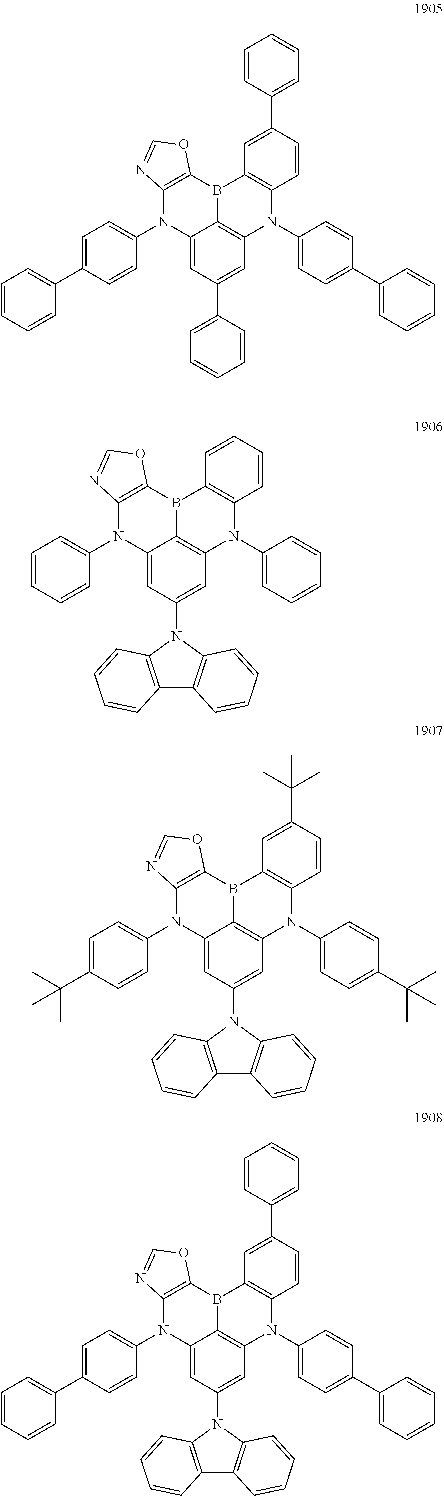 [SMILES: c1ccc(-c2ccc(N3c4ccc(-c5ccccc5)cc4B4c5ocnc5N(c5ccc(-c6ccccc6)cc5)c5cc(-c6ccccc6)cc3c54)cc2)cc1 ; c1ccc(N2c3ccccc3B3c4ocnc4N(c4ccccc4)c4cc(-n5c6ccccc6c6ccccc65)cc2c43)cc1 ; CC(C)(C)c1ccc(N2c3ccc(C(C)(C)C)cc3B3c4ocnc4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4B4c5ocnc5N(c5ccc(-c6ccccc6)cc5)c5cc(-n6c7ccccc7c7ccccc76)cc3c54)cc2)cc1]

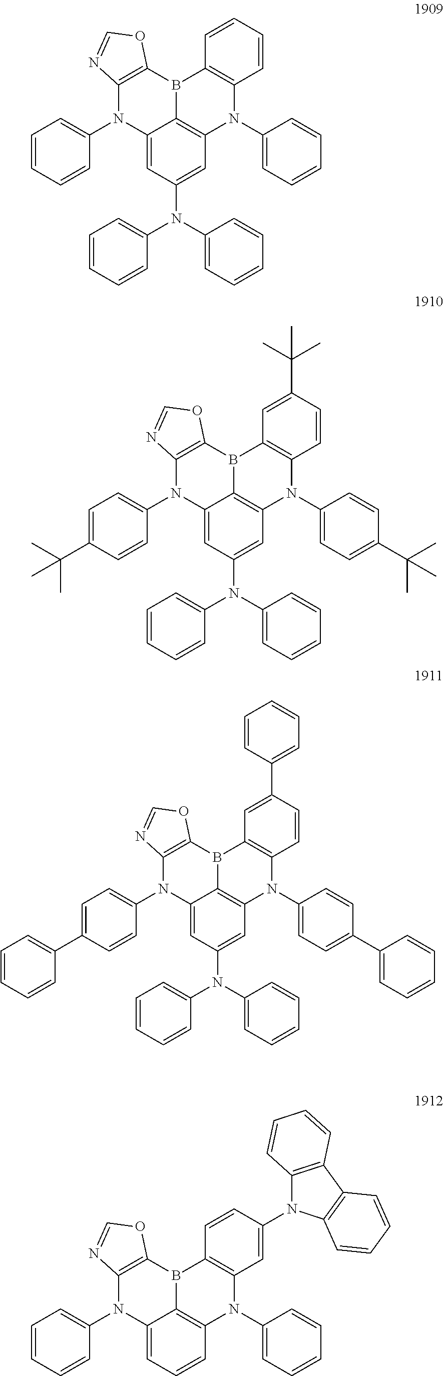

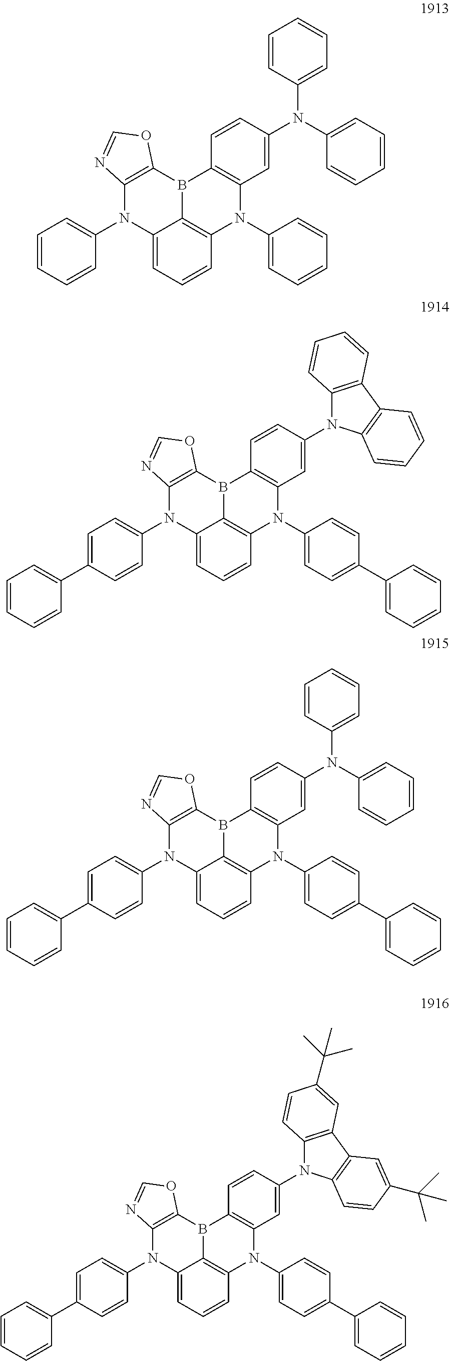 [SMILES: c1ccc(N(c2ccccc2)c2ccc3c(c2)N(c2ccccc2)c2cccc4c2B3c2ocnc2N4c2ccccc2)cc1 ; c1ccc(-c2ccc(N3c4cc(-n5c6ccccc6c6ccccc65)ccc4B4c5ocnc5N(c5ccc(-c6ccccc6)cc5)c5cccc3c54)cc2)cc1 ; c1ccc(-c2ccc(N3c4cc(N(c5ccccc5)c5ccccc5)ccc4B4c5ocnc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ocnc1N3c1ccc(-c2ccccc2)cc1]

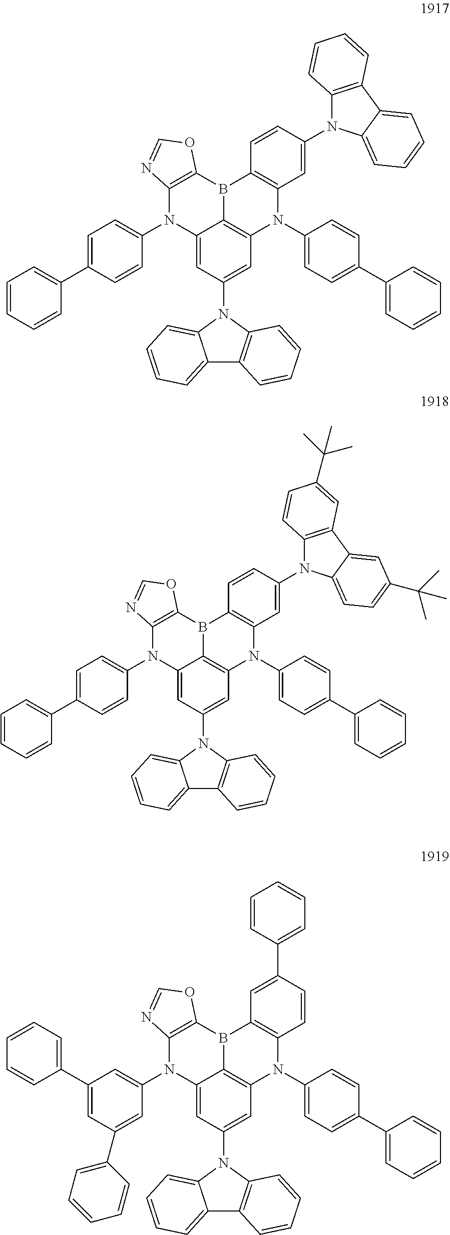 [SMILES: c1ccc(-c2ccc(N3c4cc(-n5c6ccccc6c6ccccc65)ccc4B4c5ocnc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ocnc1N3c1ccc(-c2ccccc2)cc1 ; c1ccc(-c2ccc(N3c4ccc(-c5ccccc5)cc4B4c5ocnc5N(c5cc(-c6ccccc6)cc(-c6ccccc6)c5)c5cc(-n6c7ccccc7c7ccccc76)cc3c54)cc2)cc1]

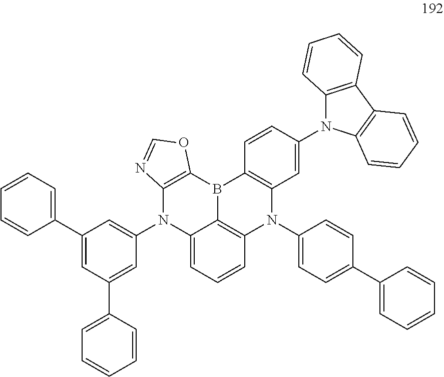 [SMILES: c1ccc(-c2ccc(N3c4cc(-n5c6ccccc6c6ccccc65)ccc4B4c5ocnc5N(c5cc(-c6ccccc6)cc(-c6ccccc6)c5)c5cccc3c54)cc2)cc1]

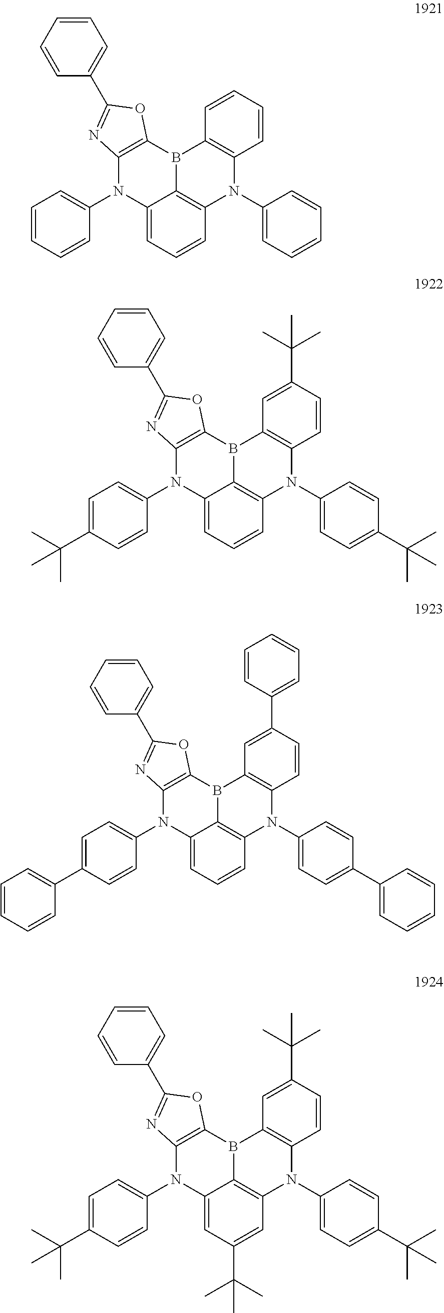

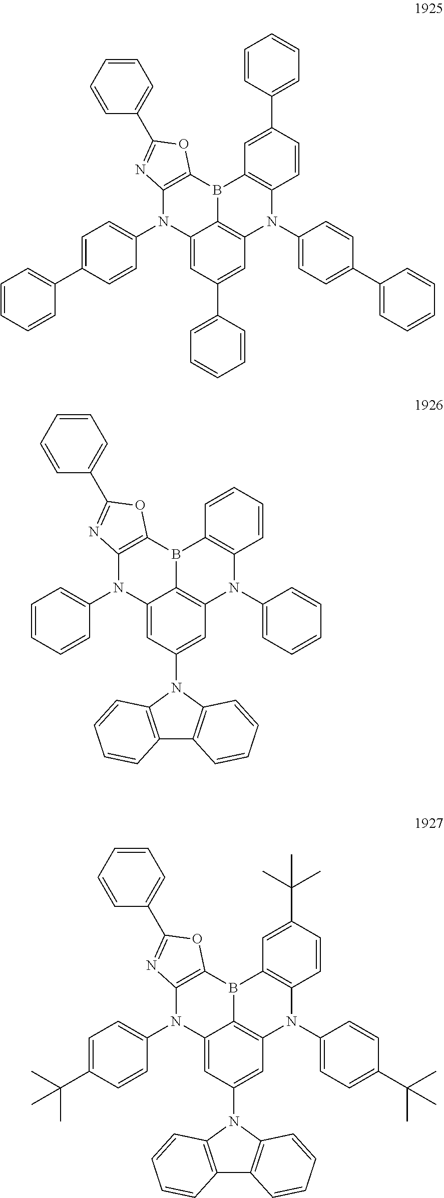 [SMILES: c1ccc(-c2ccc(N3c4ccc(-c5ccccc5)cc4B4c5oc(-c6ccccc6)nc5N(c5ccc(-c6ccccc6)cc5)c5cc(-c6ccccc6)cc3c54)cc2)cc1 ; c1ccc(-c2nc3c(o2)B2c4ccccc4N(c4ccccc4)c4cc(-n5c6ccccc6c6ccccc65)cc(c42)N3c2ccccc2)cc1 ; CC(C)(C)c1ccc(N2c3ccc(C(C)(C)C)cc3B3c4oc(-c5ccccc5)nc4N(c4ccc(C(C)(C)C)cc4)c4cc(-n5c6ccccc6c6ccccc65)cc2c43)cc1]

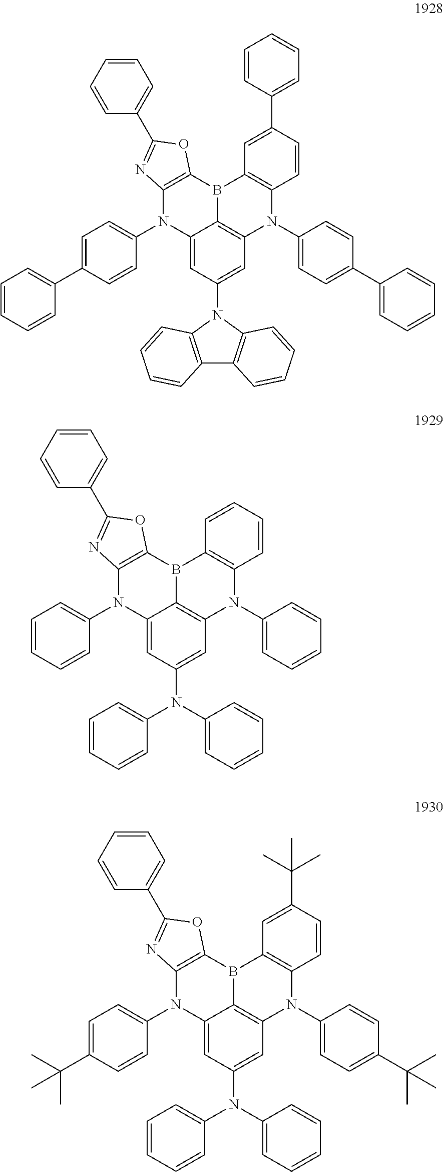 [SMILES: c1ccc(-c2ccc(N3c4ccc(-c5ccccc5)cc4B4c5oc(-c6ccccc6)nc5N(c5ccc(-c6ccccc6)cc5)c5cc(-n6c7ccccc7c7ccccc76)cc3c54)cc2)cc1 ; c1ccc(-c2nc3c(o2)B2c4ccccc4N(c4ccccc4)c4cc(N(c5ccccc5)c5ccccc5)cc(c42)N3c2ccccc2)cc1 ; CC(C)(C)c1ccc(N2c3ccc(C(C)(C)C)cc3B3c4oc(-c5ccccc5)nc4N(c4ccc(C(C)(C)C)cc4)c4cc(N(c5ccccc5)c5ccccc5)cc2c43)cc1]

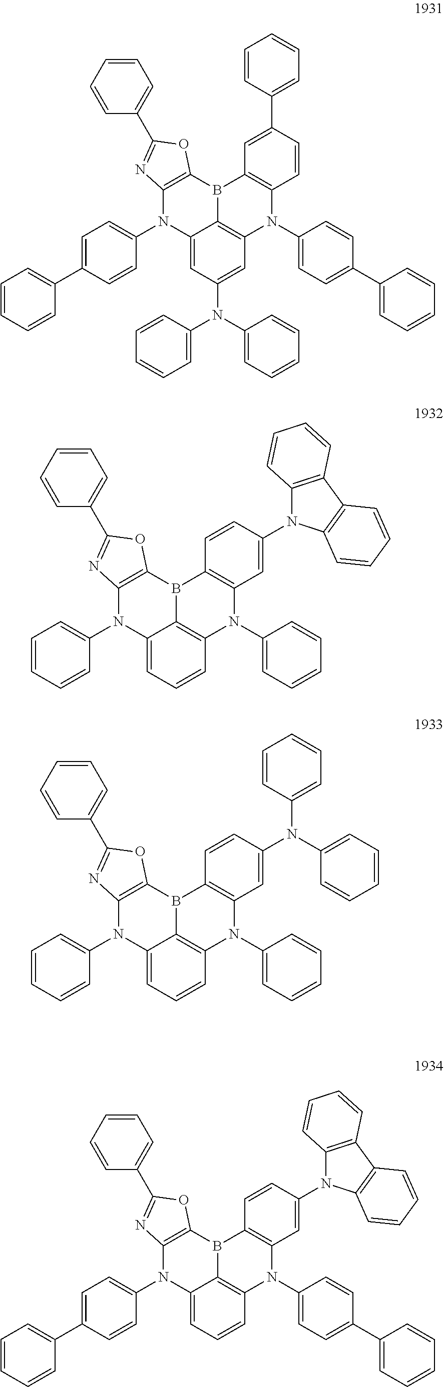 [SMILES: c1ccc(-c2ccc(N3c4ccc(-c5ccccc5)cc4B4c5oc(-c6ccccc6)nc5N(c5ccc(-c6ccccc6)cc5)c5cc(N(c6ccccc6)c6ccccc6)cc3c54)cc2)cc1 ; c1ccc(-c2nc3c(o2)B2c4ccc(-n5c6ccccc6c6ccccc65)cc4N(c4ccccc4)c4cccc(c42)N3c2ccccc2)cc1 ; c1ccc(-c2nc3c(o2)B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5oc(-c6ccccc6)nc5N(c5ccc(-c6ccccc6)cc5)c5cccc3c54)cc2)cc1]

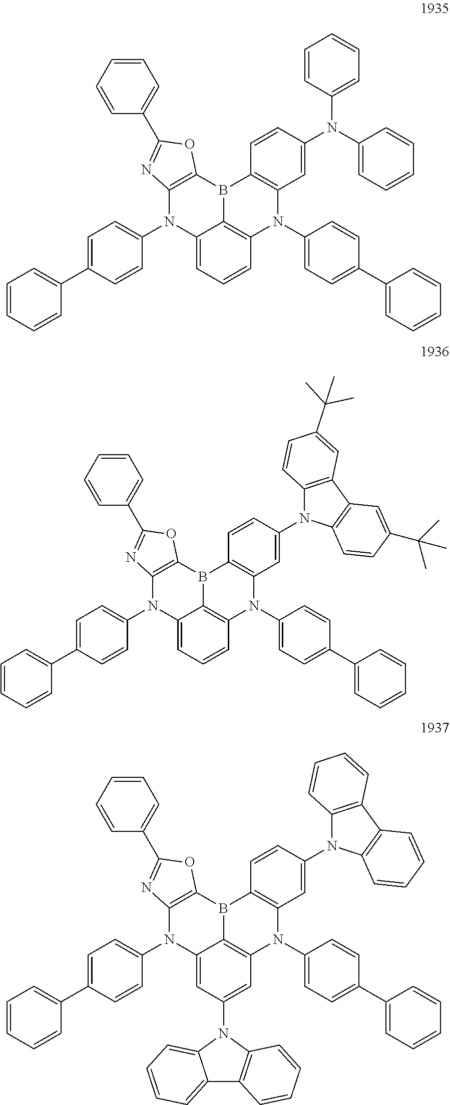 [SMILES: c1ccc(-c2ccc(N3c4cc(N(c5ccccc5)c5ccccc5)ccc4B4c5oc(-c6ccccc6)nc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1oc(-c2ccccc2)nc1N3c1ccc(-c2ccccc2)cc1 ; c1ccc(-c2ccc(N3c4cc(-n5c6ccccc6c6ccccc65)ccc4B4c5oc(-c6ccccc6)nc5N(c5ccc(-c6ccccc6)cc5)c5cc(-n6c7ccccc7c7ccccc76)cc3c54)cc2)cc1]

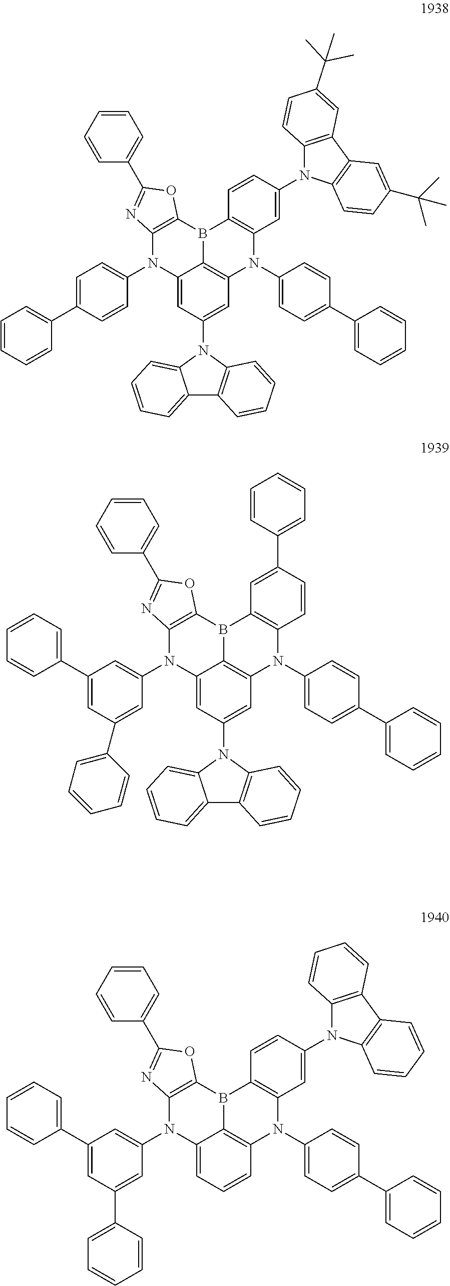 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1oc(-c2ccccc2)nc1N3c1ccc(-c2ccccc2)cc1 ; c1ccc(-c2ccc(N3c4ccc(-c5ccccc5)cc4B4c5oc(-c6ccccc6)nc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5oc(-c6ccccc6)nc5N(c5cc(-c6ccccc6)cc(-c6ccccc6)c5)c5cccc3c54)cc2)cc1]

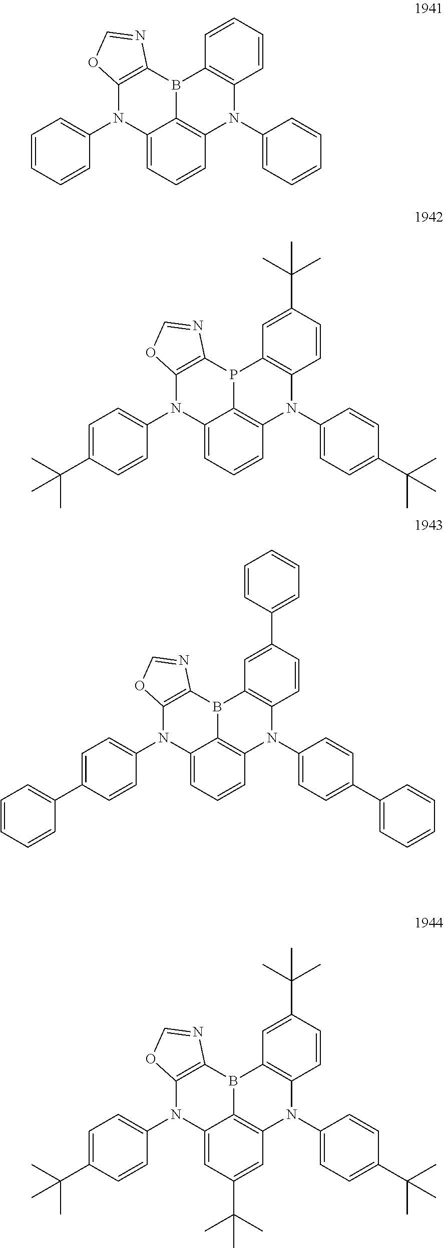

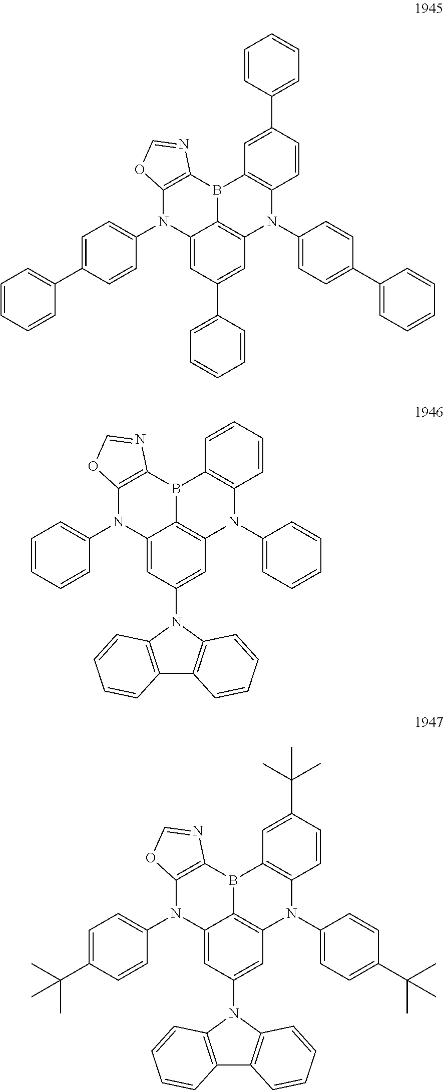 [SMILES: c1ccc(-c2ccc(N3c4ccc(-c5ccccc5)cc4B4c5ncoc5N(c5ccc(-c6ccccc6)cc5)c5cc(-c6ccccc6)cc3c54)cc2)cc1 ; c1ccc(N2c3ccccc3B3c4ncoc4N(c4ccccc4)c4cc(-n5c6ccccc6c6ccccc65)cc2c43)cc1 ; CC(C)(C)c1ccc(N2c3ccc(C(C)(C)C)cc3B3c4ncoc4N(c4ccc(C(C)(C)C)cc4)c4cc(-n5c6ccccc6c6ccccc65)cc2c43)cc1]

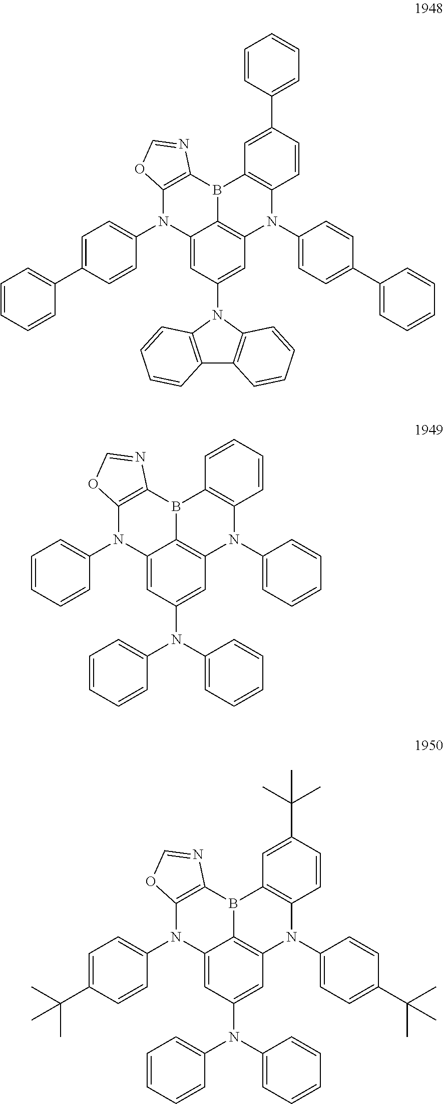 [SMILES: c1ccc(-c2ccc(N3c4ccc(-c5ccccc5)cc4B4c5ncoc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2ocnc2B4c2ccccc2N3c2ccccc2)cc1 ; CC(C)(C)c1ccc(N2c3ccc(C(C)(C)C)cc3B3c4ncoc4N(c4ccc(C(C)(C)C)cc4)c4cc(N(c5ccccc5)c5ccccc5)cc2c43)cc1]

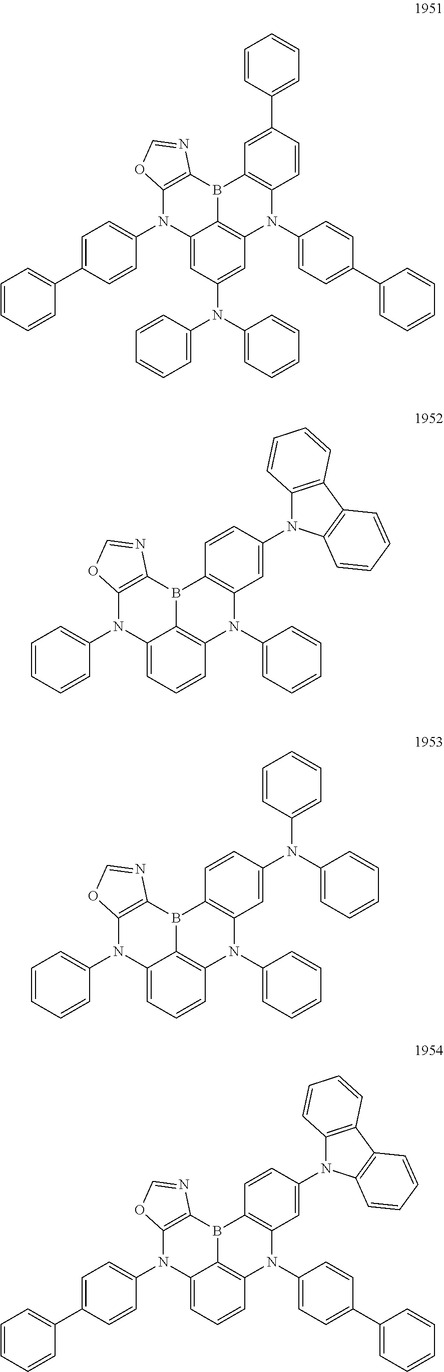 [SMILES: c1ccc(-c2ccc(N3c4ccc(-c5ccccc5)cc4B4c5ncoc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4ncoc4N(c4ccccc4)c4cccc2c43)cc1 ; c1ccc(N(c2ccccc2)c2ccc3c(c2)N(c2ccccc2)c2cccc4c2B3c2ncoc2N4c2ccccc2)cc1 ; c1ccc(-c2ccc(N3c4cc(-n5c6ccccc6c6ccccc65)ccc4B4c5ncoc5N(c5ccc(-c6ccccc6)cc5)c5cccc3c54)cc2)cc1]

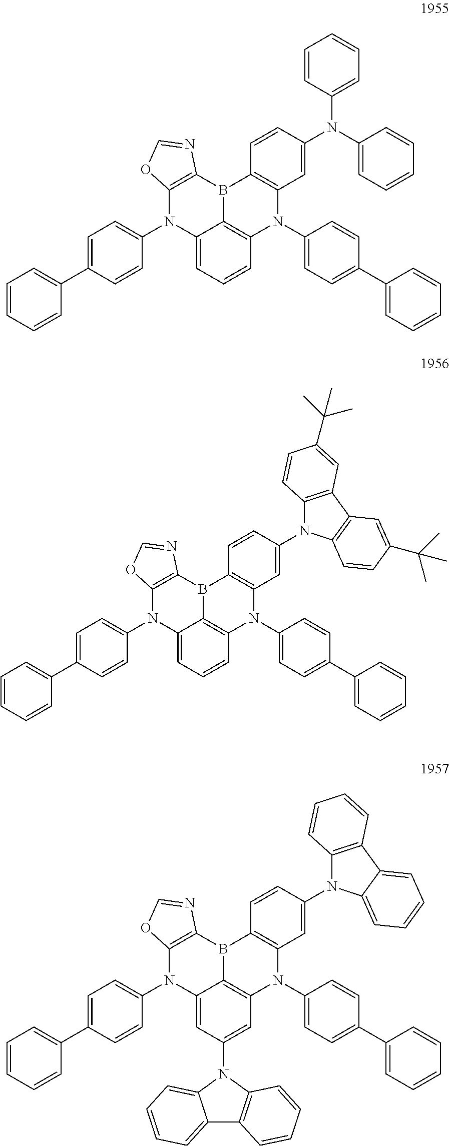 [SMILES: c1ccc(-c2ccc(N3c4cc(N(c5ccccc5)c5ccccc5)ccc4B4c5ncoc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ncoc1N3c1ccc(-c2ccccc2)cc1 ; c1ccc(-c2ccc(N3c4cc(-n5c6ccccc6c6ccccc65)ccc4B4c5ncoc5N(c5ccc(-c6ccccc6)cc5)c5cc(-n6c7ccccc7c7ccccc76)cc3c54)cc2)cc1]

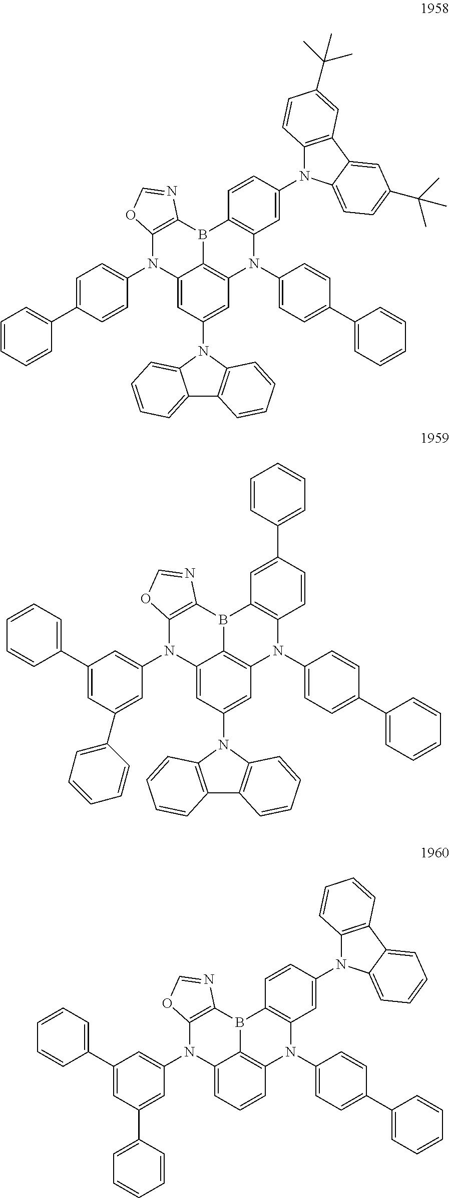 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ncoc1N3c1ccc(-c2ccccc2)cc1 ; c1ccc(-c2ccc(N3c4ccc(-c5ccccc5)cc4B4c5ncoc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ncoc5N(c5cc(-c6ccccc6)cc(-c6ccccc6)c5)c5cccc3c54)cc2)cc1]

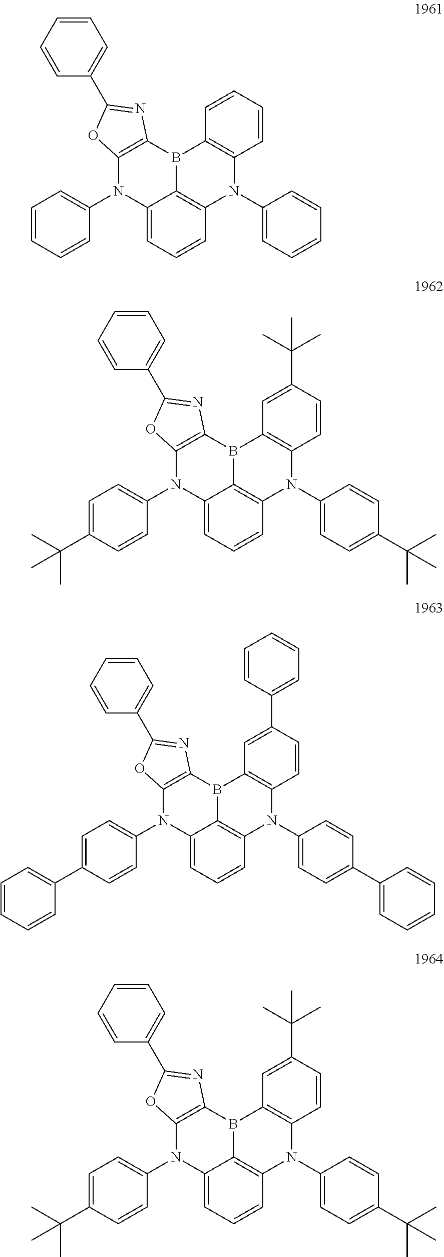

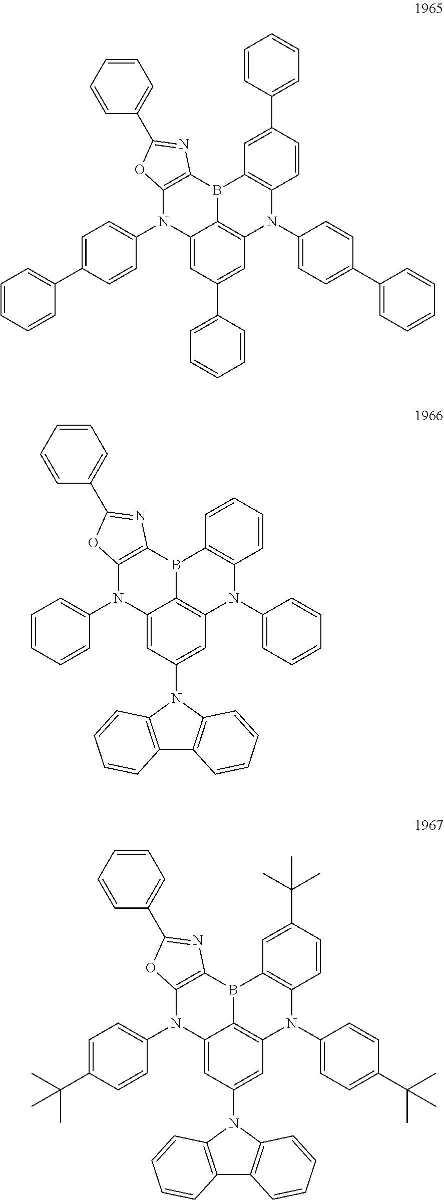 [SMILES: c1ccc(-c2ccc(N3c4ccc(-c5ccccc5)cc4B4c5nc(-c6ccccc6)oc5N(c5ccc(-c6ccccc6)cc5)c5cc(-c6ccccc6)cc3c54)cc2)cc1 ; c1ccc(-c2nc3c(o2)N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4nc(-c5ccccc5)oc4N(c4ccc(C(C)(C)C)cc4)c4cc(-n5c6ccccc6c6ccccc65)cc2c43)cc1]

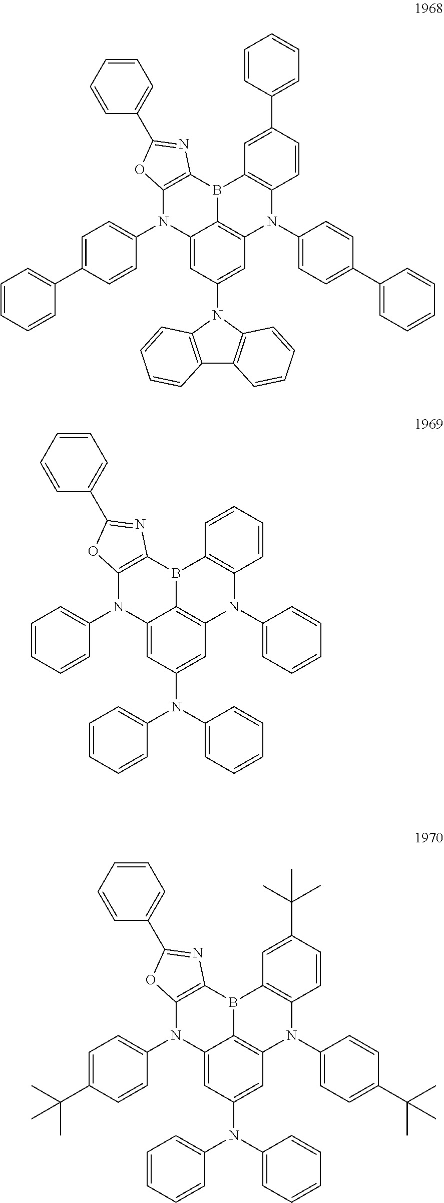 [SMILES: c1ccc(-c2ccc(N3c4ccc(-c5ccccc5)cc4B4c5nc(-c6ccccc6)oc5N(c5ccc(-c6ccccc6)cc5)c5cc(-n6c7ccccc7c7ccccc76)cc3c54)cc2)cc1 ; c1ccc(-c2nc3c(o2)N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4nc(-c5ccccc5)oc4N(c4ccc(C(C)(C)C)cc4)c4cc(N(c5ccccc5)c5ccccc5)cc2c43)cc1]

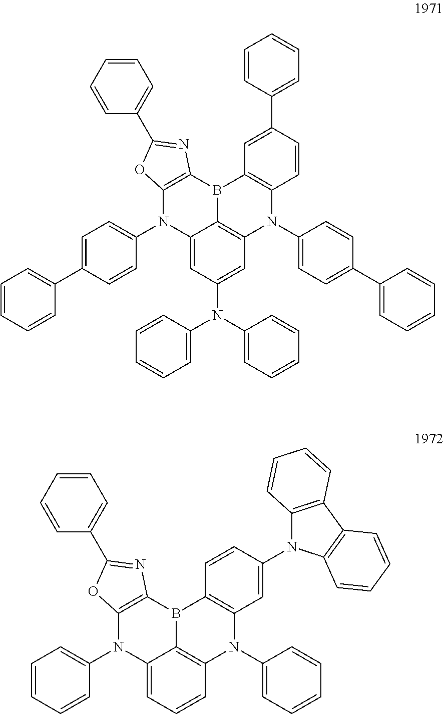 [SMILES: c1ccc(-c2ccc(N3c4ccc(-c5ccccc5)cc4B4c5nc(-c6ccccc6)oc5N(c5ccc(-c6ccccc6)cc5)c5cc(N(c6ccccc6)c6ccccc6)cc3c54)cc2)cc1 ; c1ccc(-c2nc3c(o2)N(c2ccccc2)c2cccc4c2B3c2ccc(-n3c5ccccc5c5ccccc53)cc2N4c2ccccc2)cc1]

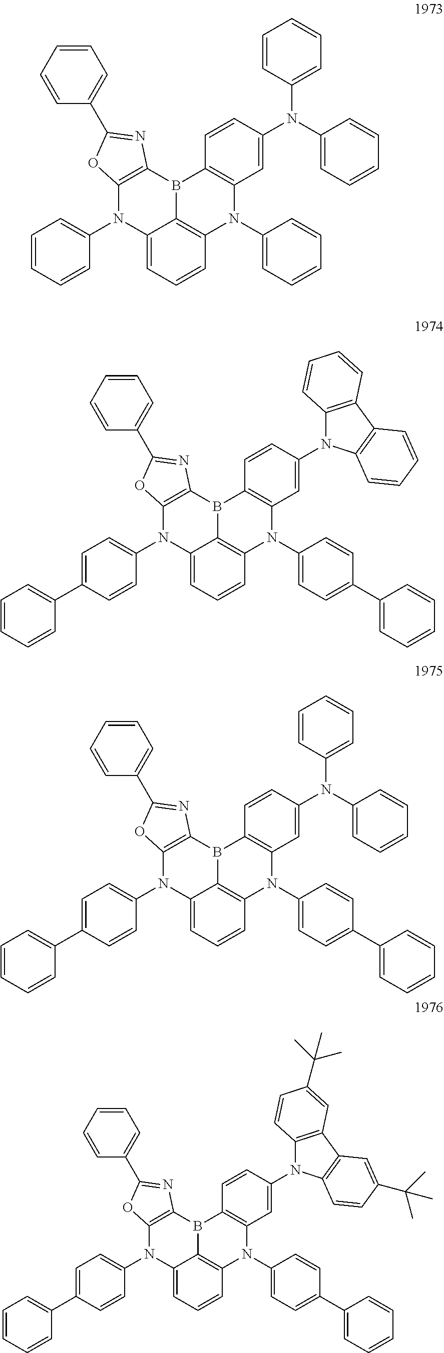 [SMILES: c1ccc(-c2nc3c(o2)N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5nc(-c6ccccc6)oc5N(c5ccc(-c6ccccc6)cc5)c5cccc3c54)cc2)cc1 ; c1ccc(-c2ccc(N3c4cc(N(c5ccccc5)c5ccccc5)ccc4B4c5nc(-c6ccccc6)oc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nc(-c2ccccc2)oc1N3c1ccc(-c2ccccc2)cc1]

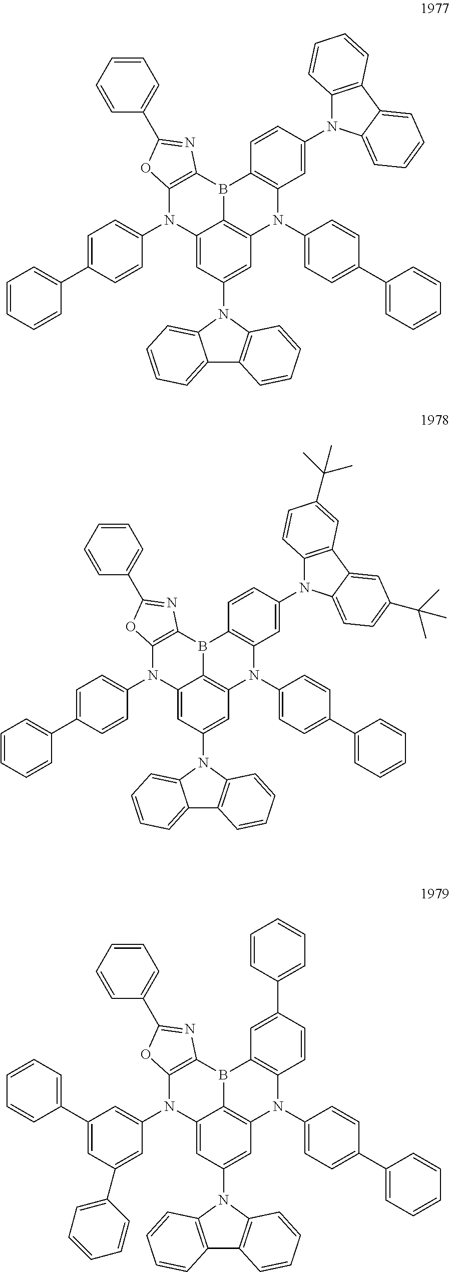 [SMILES: c1ccc(-c2ccc(N3c4cc(-n5c6ccccc6c6ccccc65)ccc4B4c5nc(-c6ccccc6)oc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nc(-c2ccccc2)oc1N3c1ccc(-c2ccccc2)cc1 ; c1ccc(-c2ccc(N3c4ccc(-c5ccccc5)cc4B4c5nc(-c6ccccc6)oc5N(c5cc(-c6ccccc6)cc(-c6ccccc6)c5)c5cc(-n6c7ccccc7c7ccccc76)cc3c54)cc2)cc1]

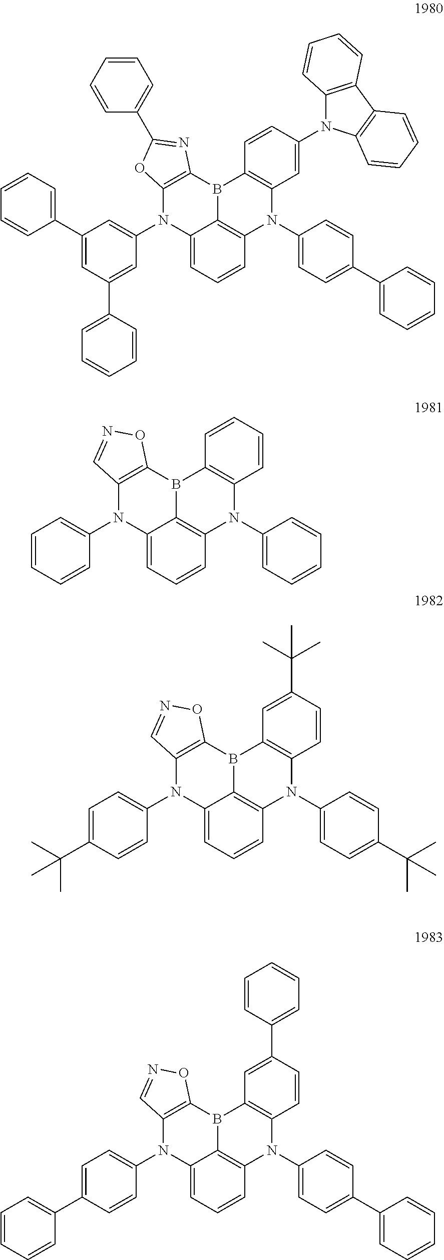

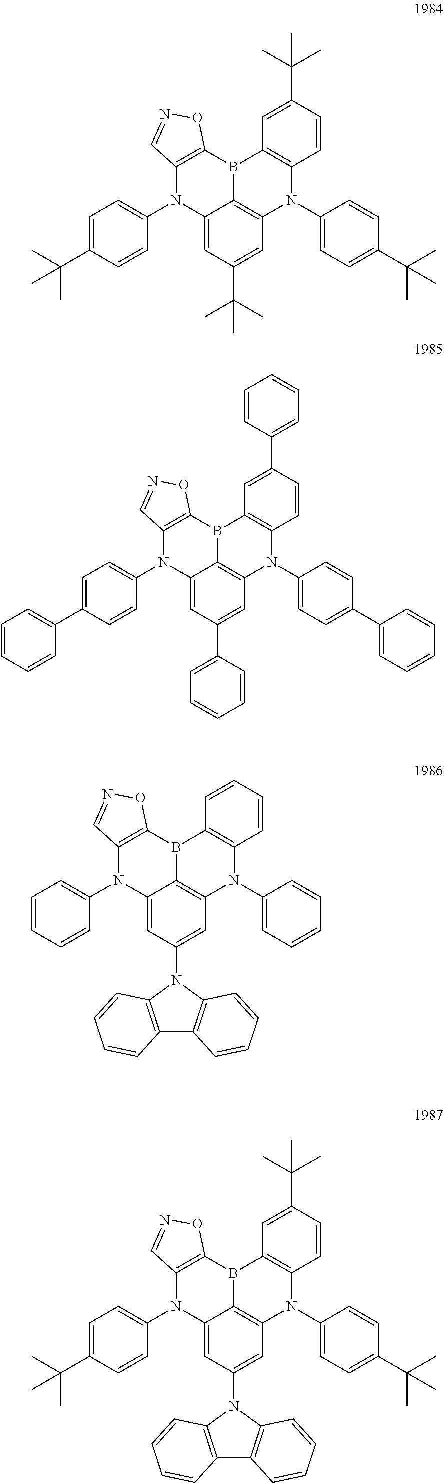

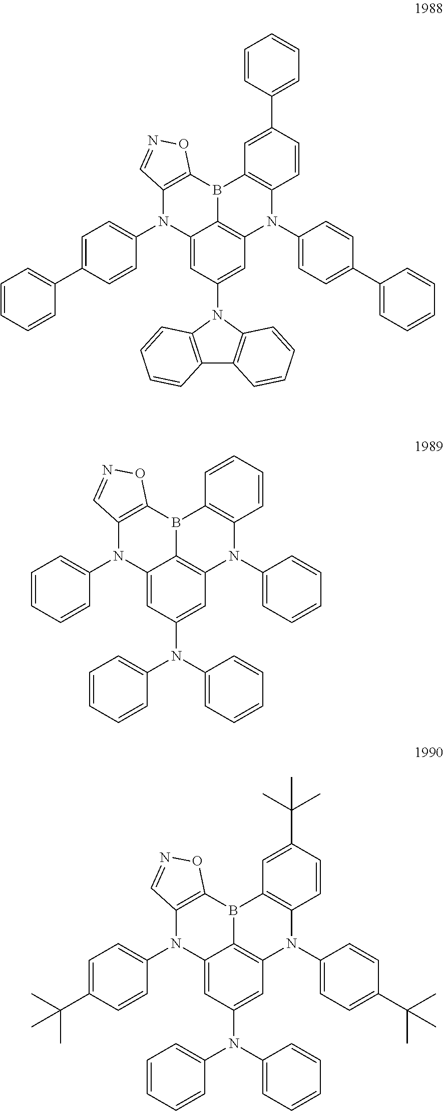 [SMILES: c1ccc(-c2ccc(N3c4ccc(-c5ccccc5)cc4B4c5oncc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2cnoc2B4c2ccccc2N3c2ccccc2)cc1 ; CC(C)(C)c1ccc(N2c3ccc(C(C)(C)C)cc3B3c4oncc4N(c4ccc(C(C)(C)C)cc4)c4cc(N(c5ccccc5)c5ccccc5)cc2c43)cc1]

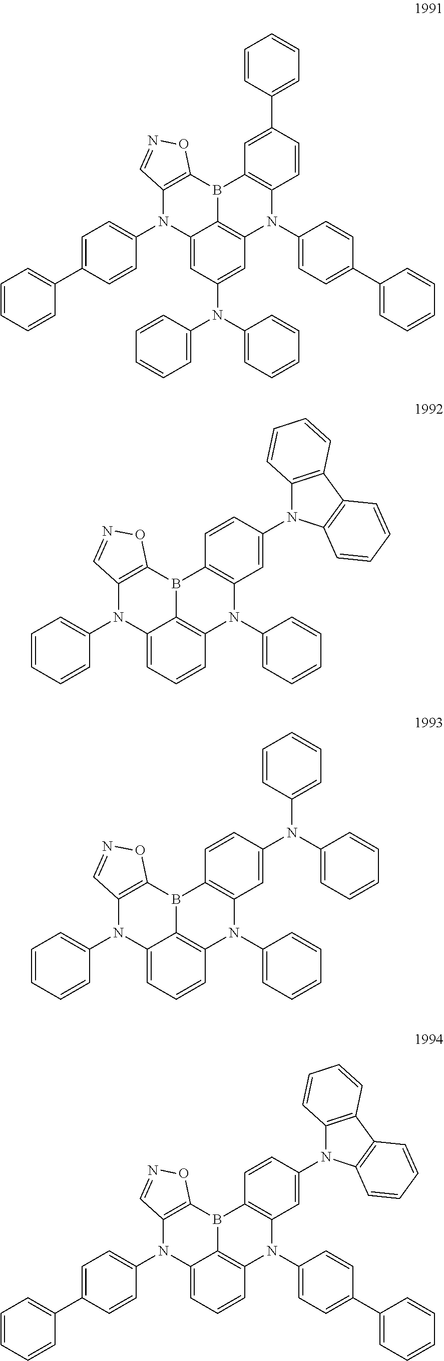 [SMILES: c1ccc(-c2ccc(N3c4ccc(-c5ccccc5)cc4B4c5oncc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4oncc4N(c4ccccc4)c4cccc2c43)cc1 ; c1ccc(N(c2ccccc2)c2ccc3c(c2)N(c2ccccc2)c2cccc4c2B3c2oncc2N4c2ccccc2)cc1 ; c1ccc(-c2ccc(N3c4cc(-n5c6ccccc6c6ccccc65)ccc4B4c5oncc5N(c5ccc(-c6ccccc6)cc5)c5cccc3c54)cc2)cc1]

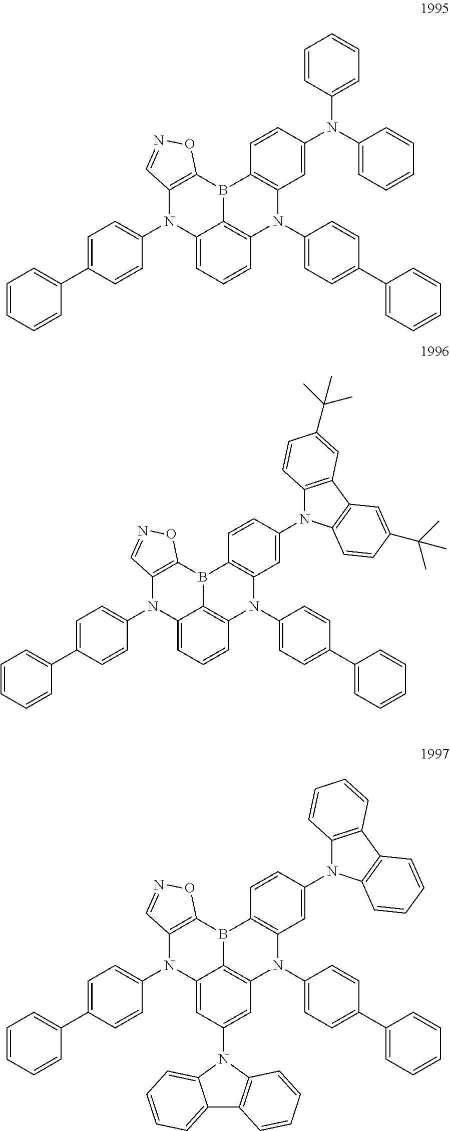 [SMILES: c1ccc(-c2ccc(N3c4cc(N(c5ccccc5)c5ccccc5)ccc4B4c5oncc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1oncc1N3c1ccc(-c2ccccc2)cc1 ; c1ccc(-c2ccc(N3c4cc(-n5c6ccccc6c6ccccc65)ccc4B4c5oncc5N(c5ccc(-c6ccccc6)cc5)c5cc(-n6c7ccccc7c7ccccc76)cc3c54)cc2)cc1]

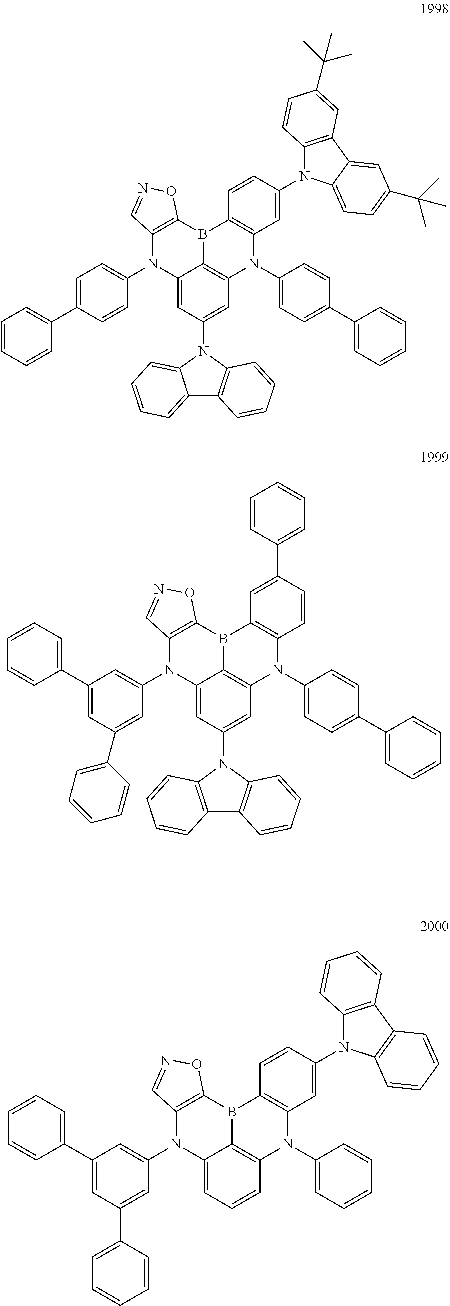 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1oncc1N3c1ccc(-c2ccccc2)cc1 ; c1ccc(-c2ccc(N3c4ccc(-c5ccccc5)cc4B4c5oncc5N(c5cc(-c6ccccc6)cc(-c6ccccc6)c5)c5cc(-n6c7ccccc7c7ccccc76)cc3c54)cc2)cc1 ; c1ccc(-c2cc(-c3ccccc3)cc(N3c4cnoc4B4c5ccc(-n6c7ccccc7c7ccccc76)cc5N(c5ccccc5)c5cccc3c54)c2)cc1]

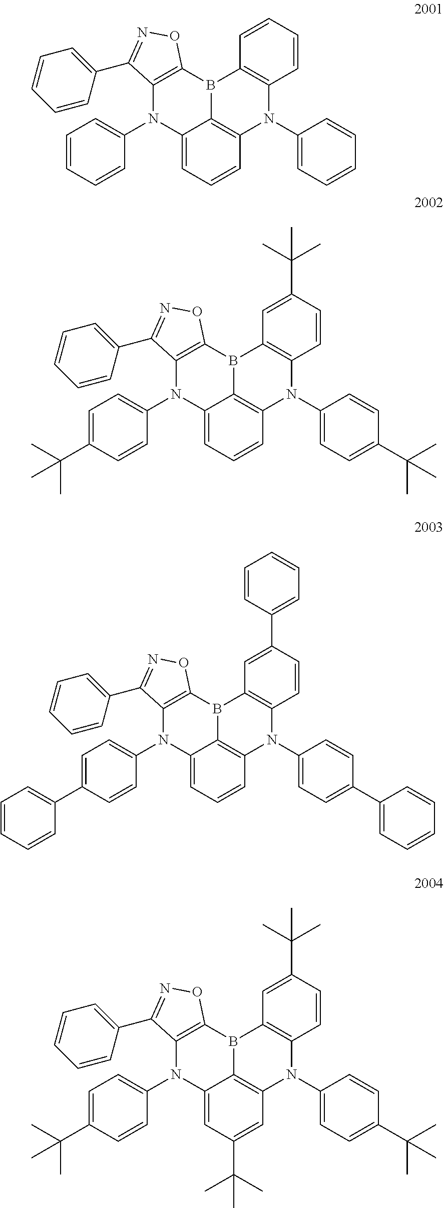

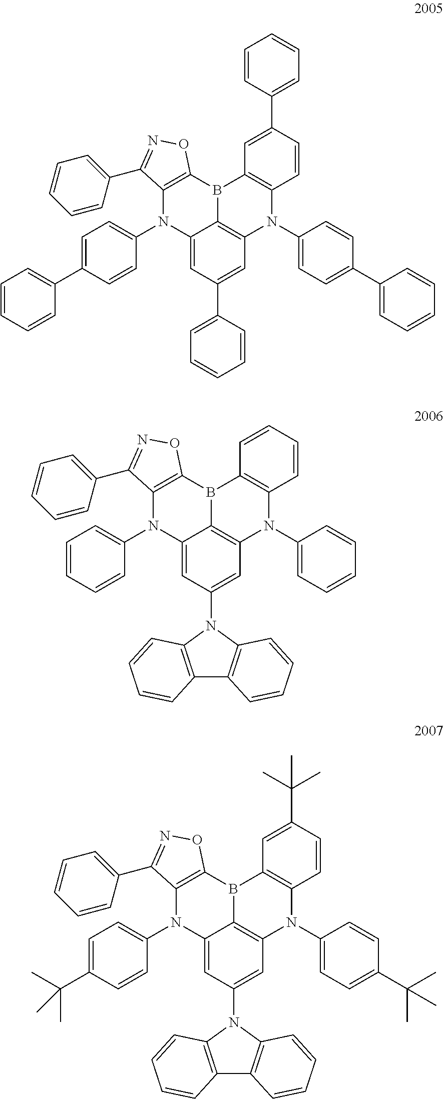 [SMILES: c1ccc(-c2ccc(N3c4ccc(-c5ccccc5)cc4B4c5onc(-c6ccccc6)c5N(c5ccc(-c6ccccc6)cc5)c5cc(-c6ccccc6)cc3c54)cc2)cc1 ; c1ccc(-c2noc3c2N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4onc(-c5ccccc5)c4N(c4ccc(C(C)(C)C)cc4)c4cc(-n5c6ccccc6c6ccccc65)cc2c43)cc1]

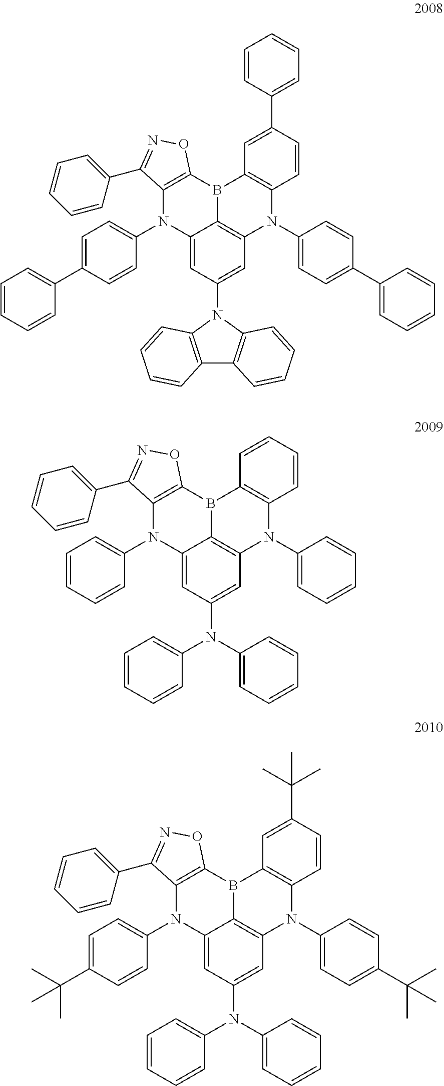 [SMILES: c1ccc(-c2ccc(N3c4ccc(-c5ccccc5)cc4B4c5onc(-c6ccccc6)c5N(c5ccc(-c6ccccc6)cc5)c5cc(-n6c7ccccc7c7ccccc76)cc3c54)cc2)cc1 ; c1ccc(-c2noc3c2N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4onc(-c5ccccc5)c4N(c4ccc(C(C)(C)C)cc4)c4cc(N(c5ccccc5)c5ccccc5)cc2c43)cc1]

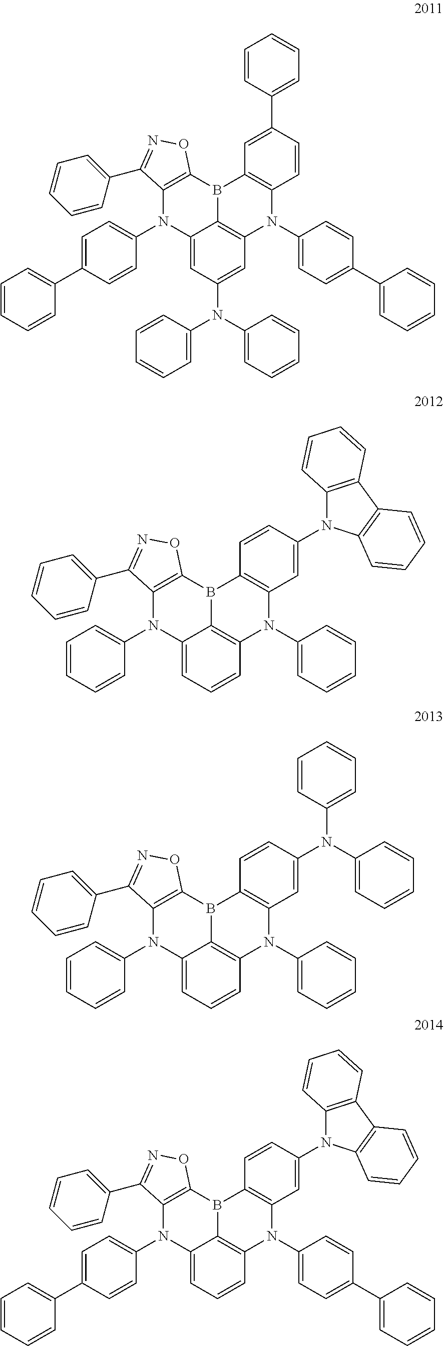

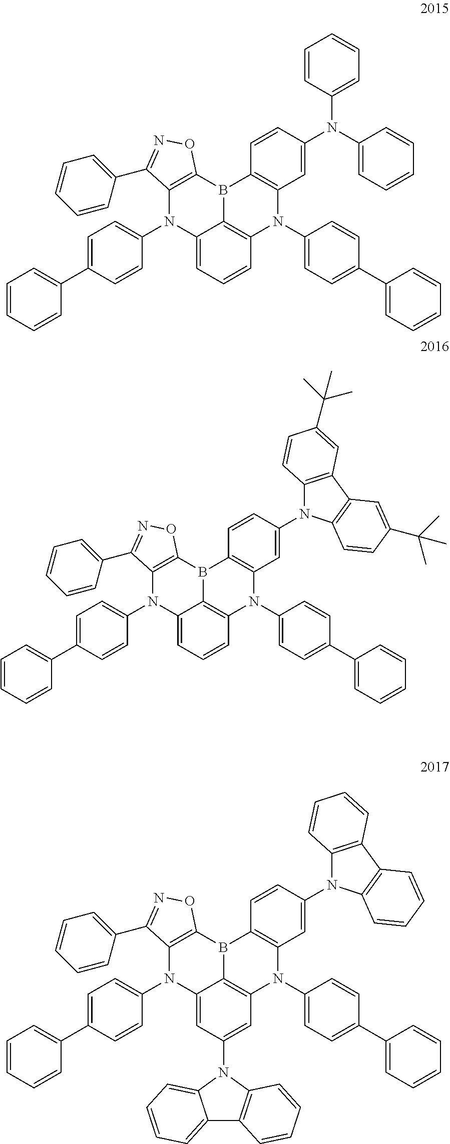 [SMILES: c1ccc(-c2ccc(N3c4cc(N(c5ccccc5)c5ccccc5)ccc4B4c5onc(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1onc(-c2ccccc2)c1N3c1ccc(-c2ccccc2)cc1 ; c1ccc(-c2ccc(N3c4cc(-n5c6ccccc6c6ccccc65)ccc4B4c5onc(-c6ccccc6)c5N(c5ccc(-c6ccccc6)cc5)c5cc(-n6c7ccccc7c7ccccc76)cc3c54)cc2)cc1]

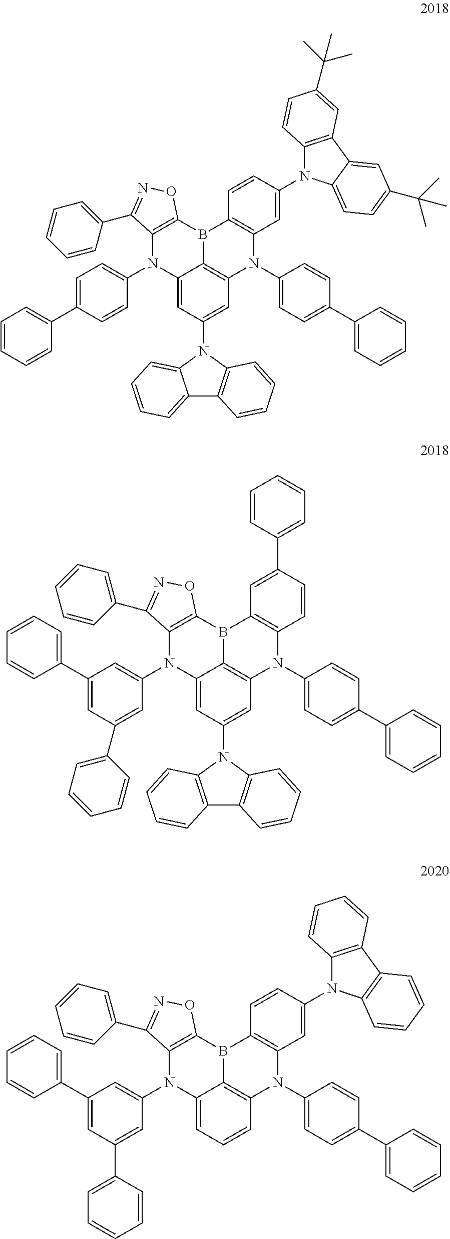 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1onc(-c2ccccc2)c1N3c1ccc(-c2ccccc2)cc1 ; c1ccc(-c2ccc(N3c4ccc(-c5ccccc5)cc4B4c5onc(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5onc(-c6ccccc6)c5N(c5cc(-c6ccccc6)cc(-c6ccccc6)c5)c5cccc3c54)cc2)cc1]

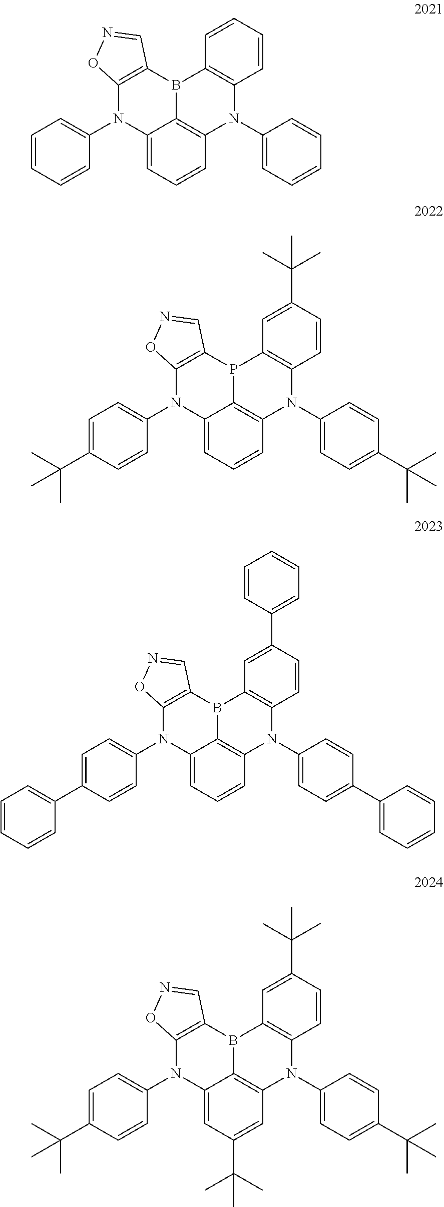

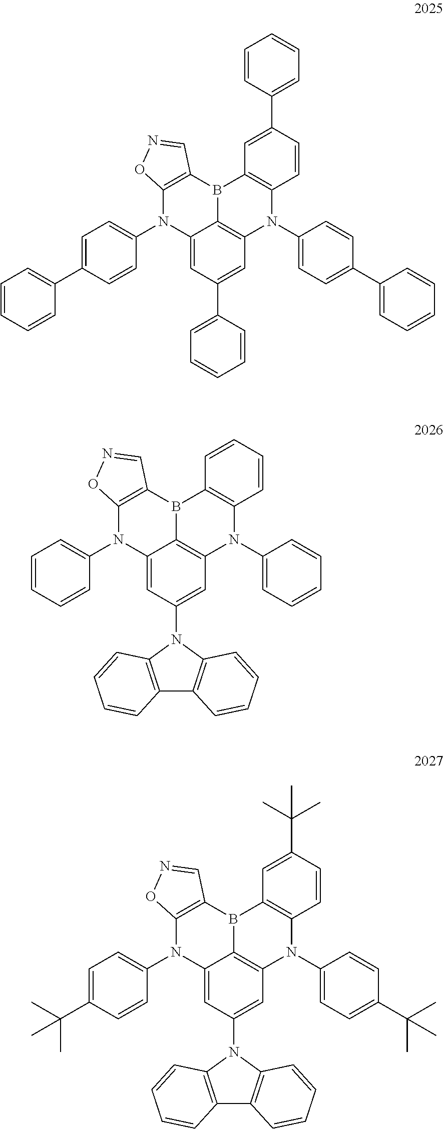 [SMILES: c1ccc(-c2ccc(N3c4ccc(-c5ccccc5)cc4B4c5cnoc5N(c5ccc(-c6ccccc6)cc5)c5cc(-c6ccccc6)cc3c54)cc2)cc1 ; c1ccc(N2c3ccccc3B3c4cnoc4N(c4ccccc4)c4cc(-n5c6ccccc6c6ccccc65)cc2c43)cc1 ; CC(C)(C)c1ccc(N2c3ccc(C(C)(C)C)cc3B3c4cnoc4N(c4ccc(C(C)(C)C)cc4)c4cc(-n5c6ccccc6c6ccccc65)cc2c43)cc1]

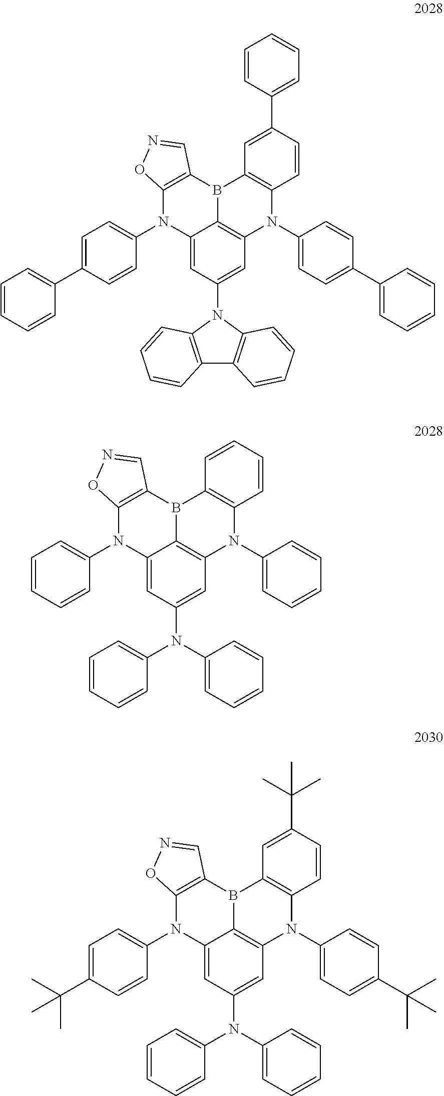 [SMILES: c1ccc(-c2ccc(N3c4ccc(-c5ccccc5)cc4B4c5cnoc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2oncc2B4c2ccccc2N3c2ccccc2)cc1 ; CC(C)(C)c1ccc(N2c3ccc(C(C)(C)C)cc3B3c4cnoc4N(c4ccc(C(C)(C)C)cc4)c4cc(N(c5ccccc5)c5ccccc5)cc2c43)cc1]

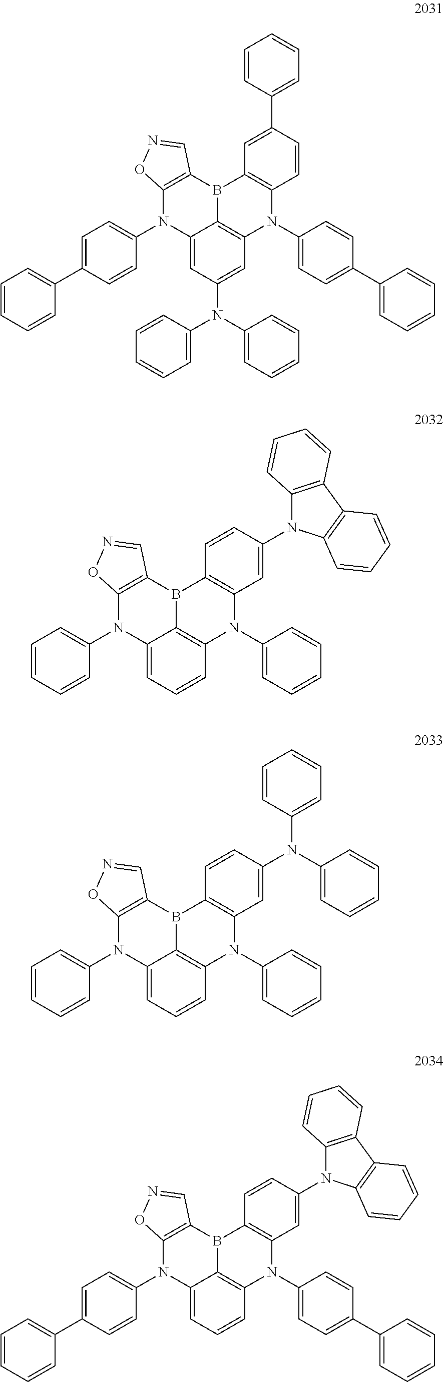 [SMILES: c1ccc(-c2ccc(N3c4ccc(-c5ccccc5)cc4B4c5cnoc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4cnoc4N(c4ccccc4)c4cccc2c43)cc1 ; c1ccc(N(c2ccccc2)c2ccc3c(c2)N(c2ccccc2)c2cccc4c2B3c2cnoc2N4c2ccccc2)cc1 ; c1ccc(-c2ccc(N3c4cc(-n5c6ccccc6c6ccccc65)ccc4B4c5cnoc5N(c5ccc(-c6ccccc6)cc5)c5cccc3c54)cc2)cc1]

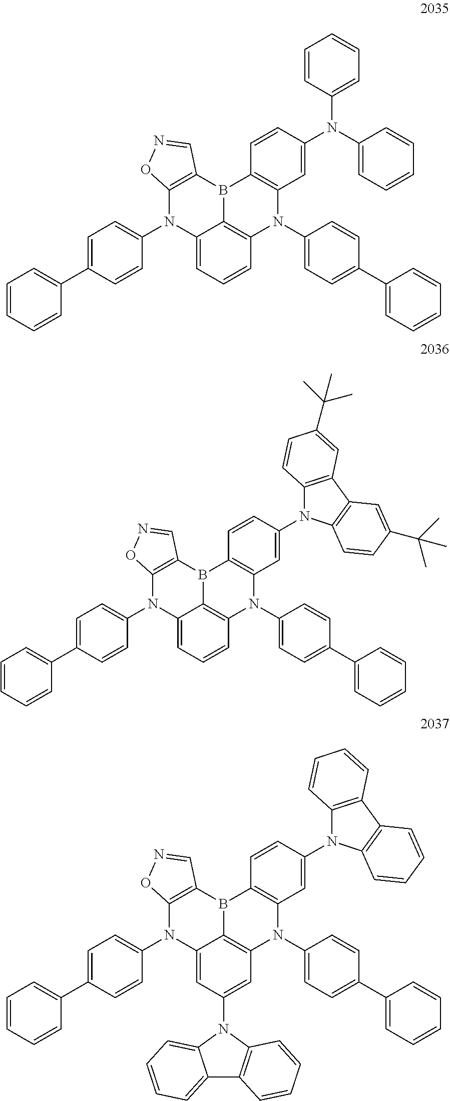 [SMILES: c1ccc(-c2ccc(N3c4cc(N(c5ccccc5)c5ccccc5)ccc4B4c5cnoc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1cnoc1N3c1ccc(-c2ccccc2)cc1 ; c1ccc(-c2ccc(N3c4cc(-n5c6ccccc6c6ccccc65)ccc4B4c5cnoc5N(c5ccc(-c6ccccc6)cc5)c5cc(-n6c7ccccc7c7ccccc76)cc3c54)cc2)cc1]

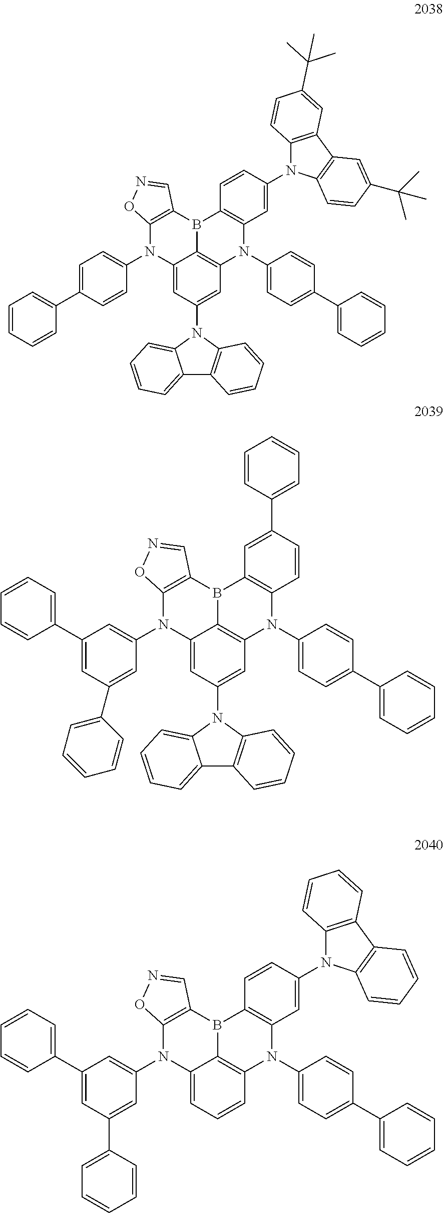 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1cnoc1N3c1ccc(-c2ccccc2)cc1 ; c1ccc(-c2ccc(N3c4ccc(-c5ccccc5)cc4B4c5cnoc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5cnoc5N(c5cc(-c6ccccc6)cc(-c6ccccc6)c5)c5cccc3c54)cc2)cc1]

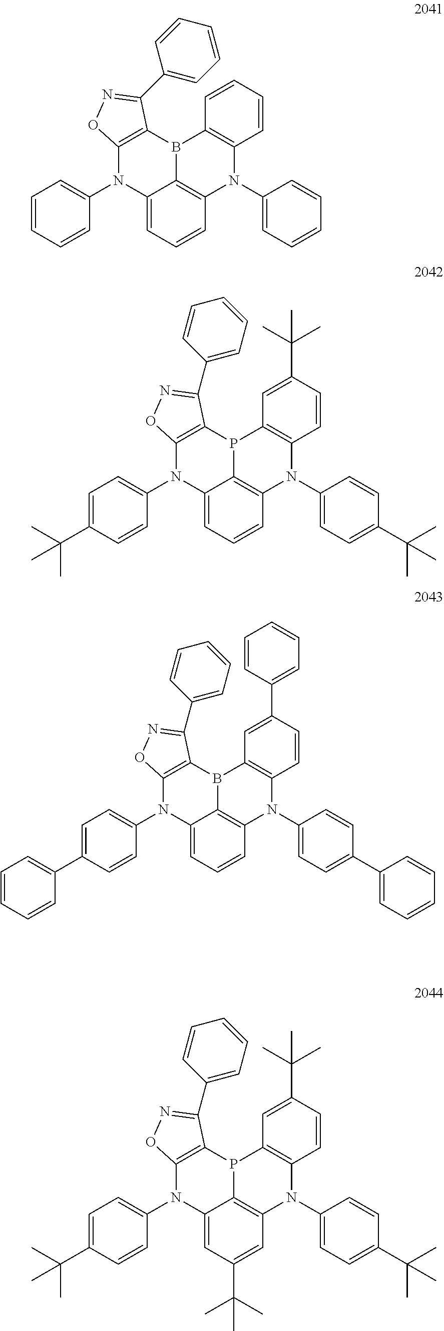

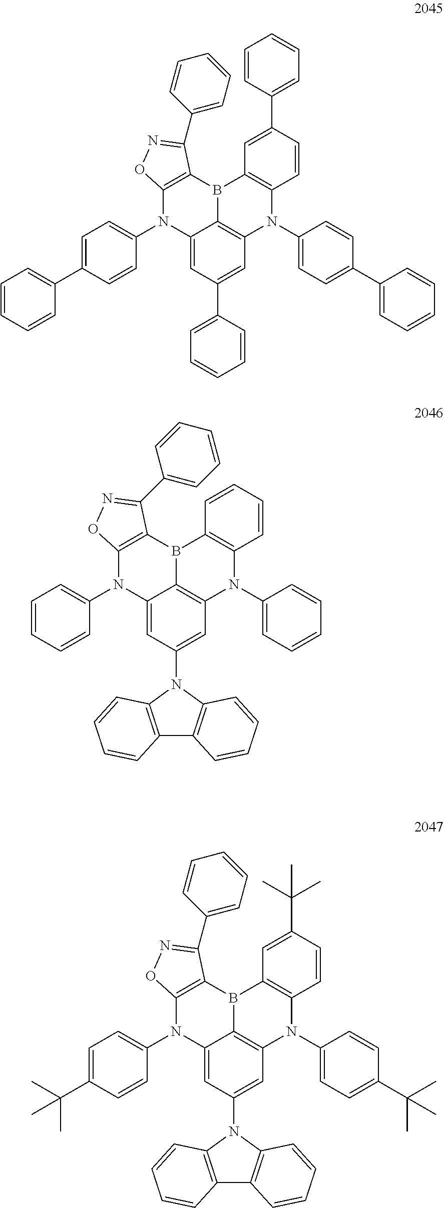 [SMILES: c1ccc(-c2ccc(N3c4ccc(-c5ccccc5)cc4B4c5c3cc(-c3ccccc3)cc5N(c3ccc(-c5ccccc5)cc3)c3onc(-c5ccccc5)c34)cc2)cc1 ; c1ccc(-c2noc3c2B2c4ccccc4N(c4ccccc4)c4cc(-n5c6ccccc6c6ccccc65)cc(c42)N3c2ccccc2)cc1 ; CC(C)(C)c1ccc(N2c3ccc(C(C)(C)C)cc3B3c4c2cc(-n2c5ccccc5c5ccccc52)cc4N(c2ccc(C(C)(C)C)cc2)c2onc(-c4ccccc4)c23)cc1]

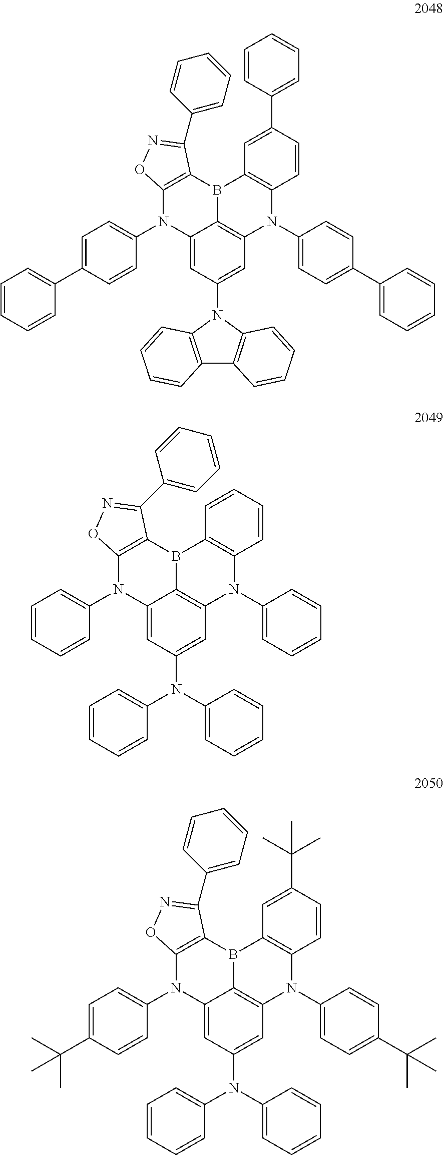

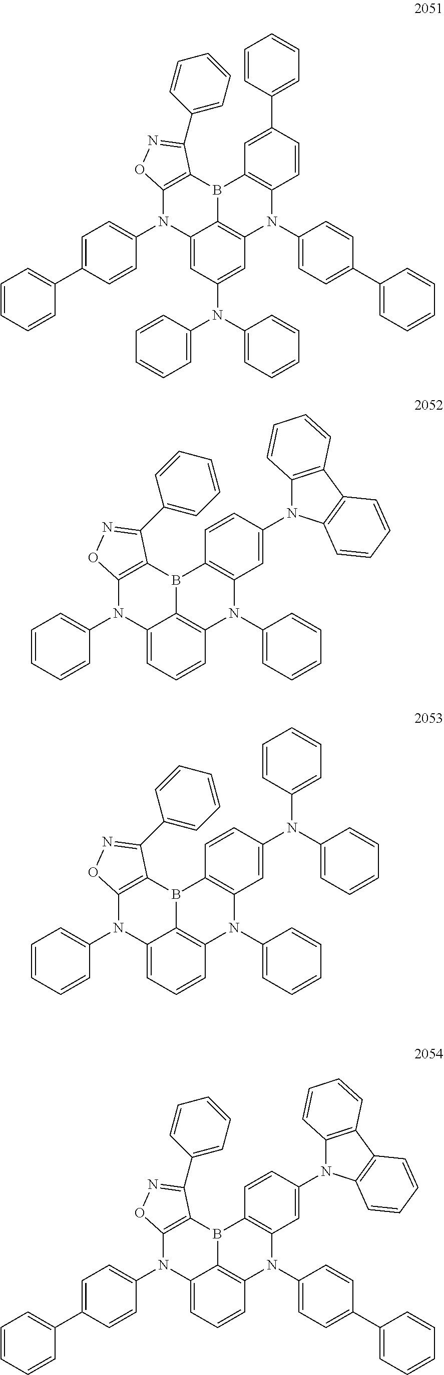 [SMILES: c1ccc(-c2ccc(N3c4ccc(-c5ccccc5)cc4B4c5c3cc(N(c3ccccc3)c3ccccc3)cc5N(c3ccc(-c5ccccc5)cc3)c3onc(-c5ccccc5)c34)cc2)cc1 ; c1ccc(-c2noc3c2B2c4ccc(-n5c6ccccc6c6ccccc65)cc4N(c4ccccc4)c4cccc(c42)N3c2ccccc2)cc1 ; c1ccc(-c2noc3c2B2c4ccc(N(c5ccccc5)c5ccccc5)cc4N(c4ccccc4)c4cccc(c42)N3c2ccccc2)cc1 ; c1ccc(-c2ccc(N3c4cc(-n5c6ccccc6c6ccccc65)ccc4B4c5c3cccc5N(c3ccc(-c5ccccc5)cc3)c3onc(-c5ccccc5)c34)cc2)cc1]

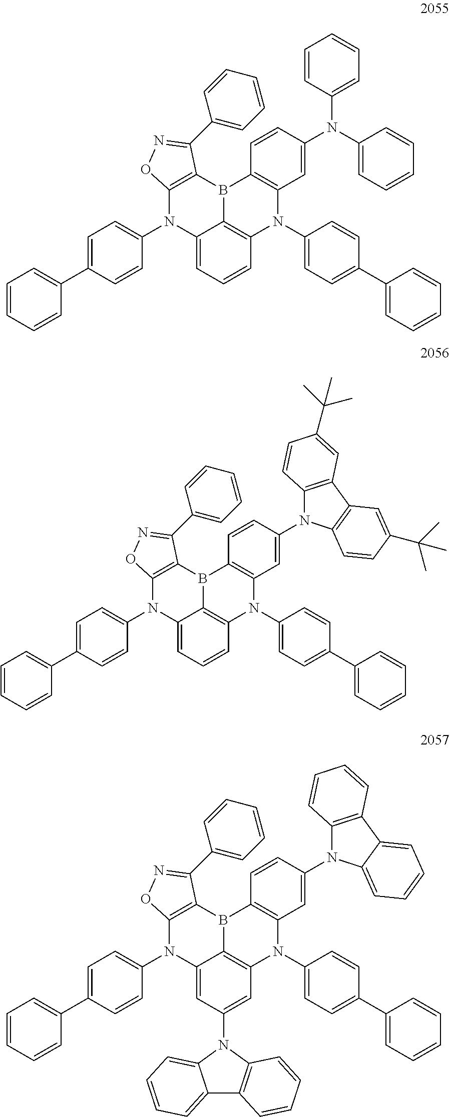 [SMILES: c1ccc(-c2ccc(N3c4cc(N(c5ccccc5)c5ccccc5)ccc4B4c5c3cccc5N(c3ccc(-c5ccccc5)cc3)c3onc(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(-c2ccccc2)noc1N3c1ccc(-c2ccccc2)cc1 ; c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3onc(-c5ccccc5)c34)cc2)cc1]

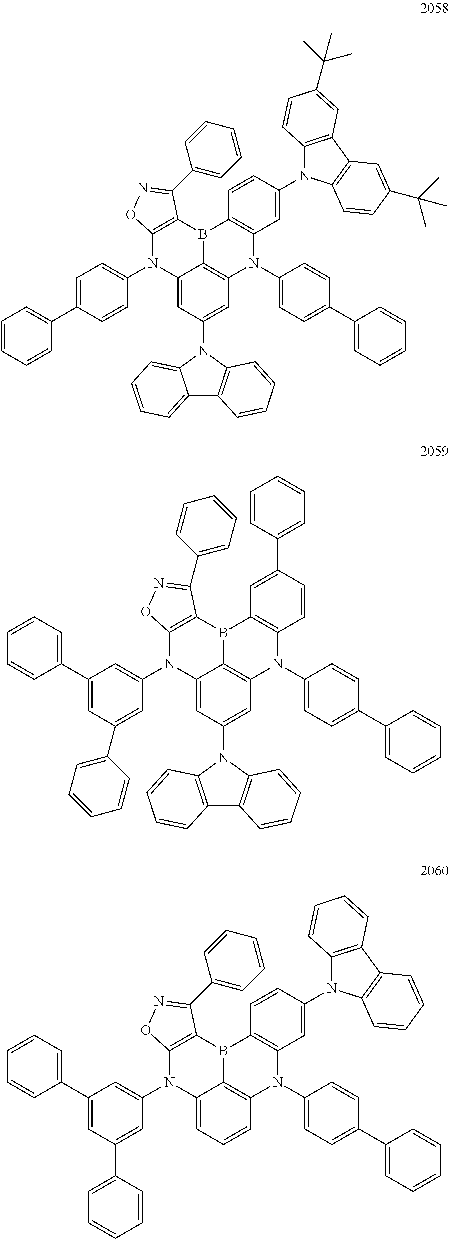 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(-c2ccccc2)noc1N3c1ccc(-c2ccccc2)cc1 ; c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3onc(-c5ccccc5)c34)cc2)cc1 ; c1ccc(-c2ccc(N3c4cc(-n5c6ccccc6c6ccccc65)ccc4B4c5c3cccc5N(c3cc(-c5ccccc5)cc(-c5ccccc5)c3)c3onc(-c5ccccc5)c34)cc2)cc1]

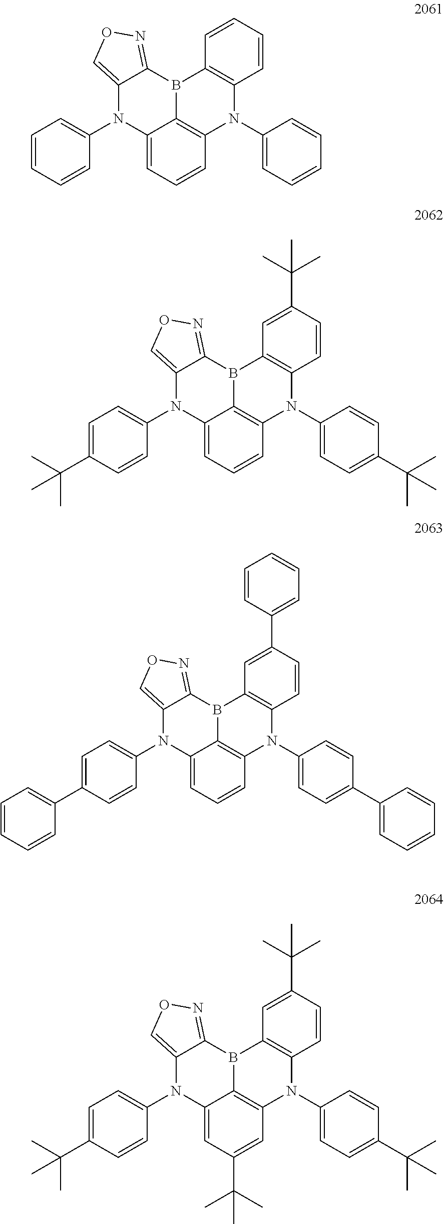

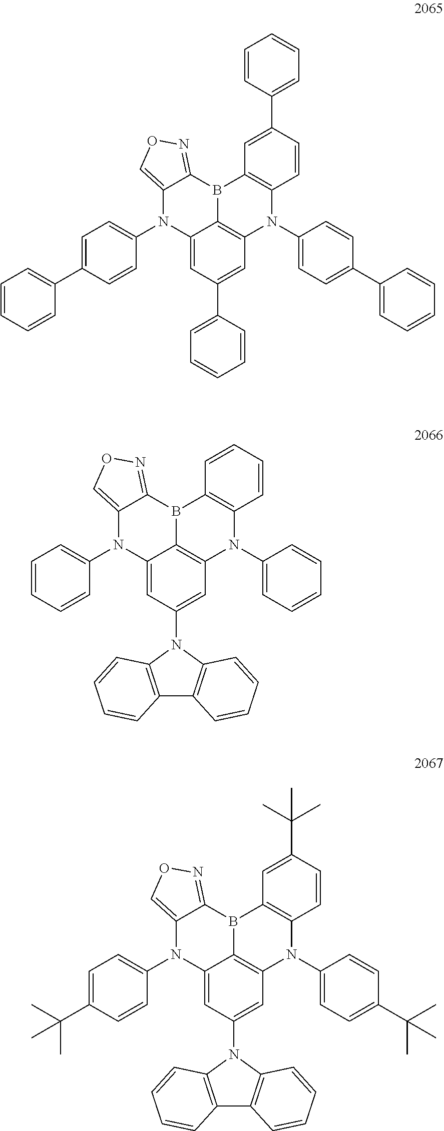 [SMILES: c1ccc(-c2ccc(N3c4ccc(-c5ccccc5)cc4B4c5nocc5N(c5ccc(-c6ccccc6)cc5)c5cc(-c6ccccc6)cc3c54)cc2)cc1 ; c1ccc(N2c3ccccc3B3c4nocc4N(c4ccccc4)c4cc(-n5c6ccccc6c6ccccc65)cc2c43)cc1 ; CC(C)(C)c1ccc(N2c3ccc(C(C)(C)C)cc3B3c4nocc4N(c4ccc(C(C)(C)C)cc4)c4cc(-n5c6ccccc6c6ccccc65)cc2c43)cc1]

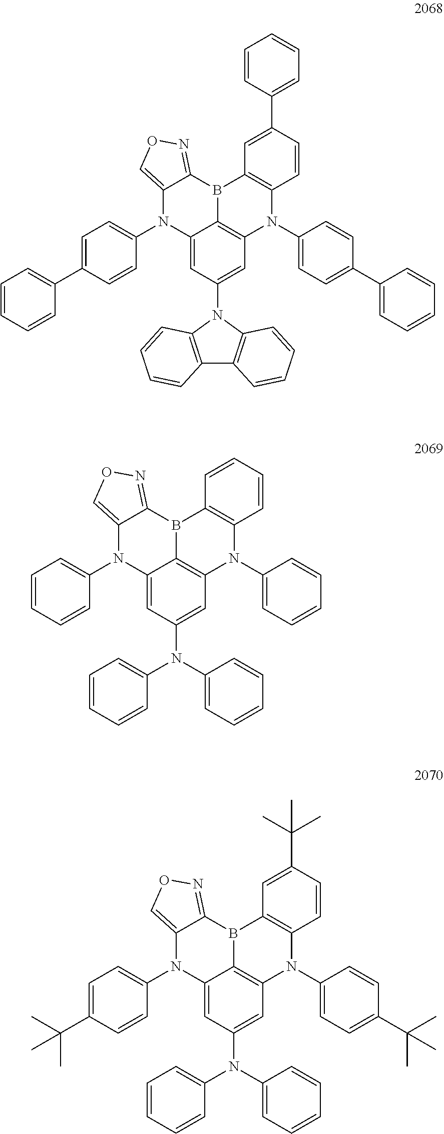 [SMILES: c1ccc(-c2ccc(N3c4ccc(-c5ccccc5)cc4B4c5nocc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2conc2B4c2ccccc2N3c2ccccc2)cc1 ; CC(C)(C)c1ccc(N2c3ccc(C(C)(C)C)cc3B3c4nocc4N(c4ccc(C(C)(C)C)cc4)c4cc(N(c5ccccc5)c5ccccc5)cc2c43)cc1]

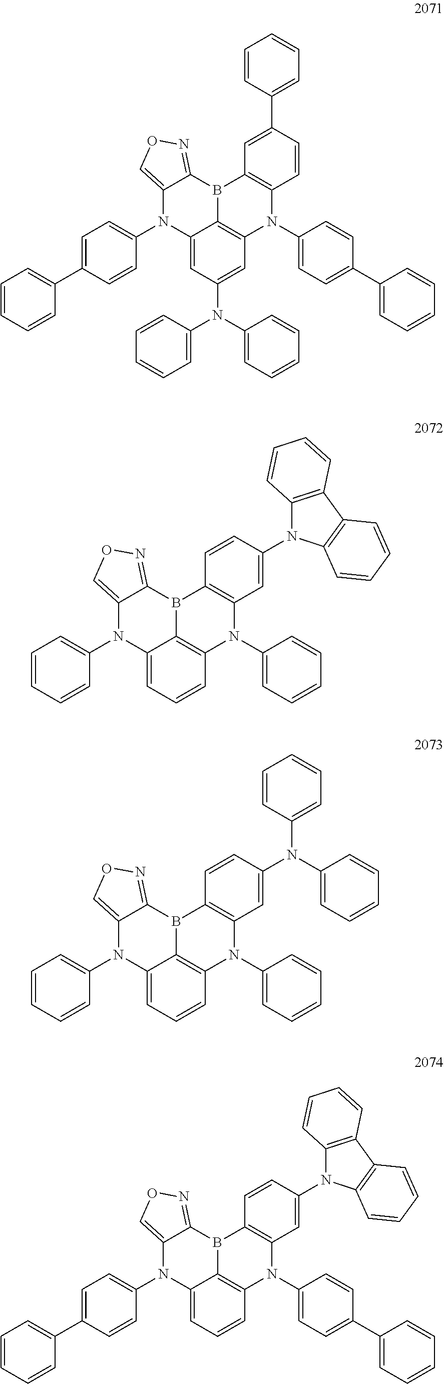 [SMILES: c1ccc(-c2ccc(N3c4ccc(-c5ccccc5)cc4B4c5nocc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nocc4N(c4ccccc4)c4cccc2c43)cc1 ; c1ccc(N(c2ccccc2)c2ccc3c(c2)N(c2ccccc2)c2cccc4c2B3c2nocc2N4c2ccccc2)cc1 ; c1ccc(-c2ccc(N3c4cc(-n5c6ccccc6c6ccccc65)ccc4B4c5nocc5N(c5ccc(-c6ccccc6)cc5)c5cccc3c54)cc2)cc1]

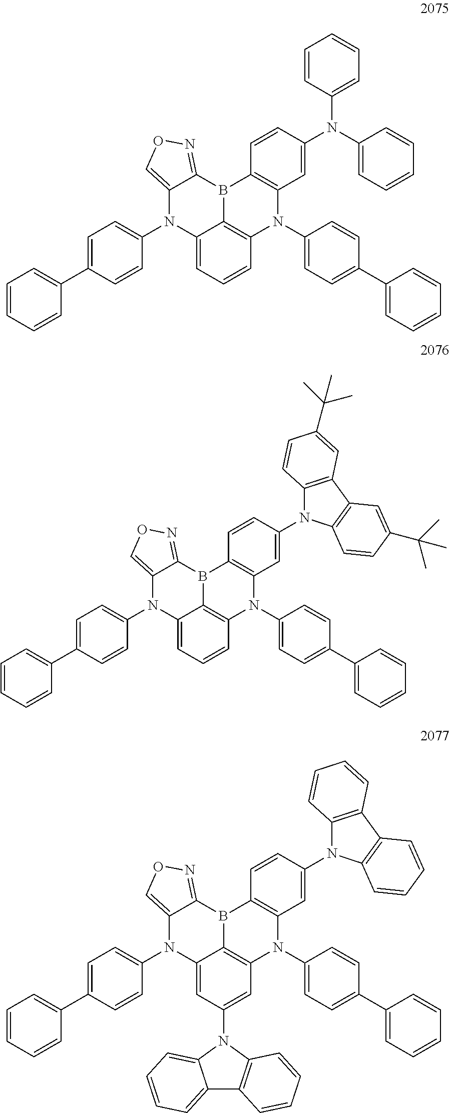 [SMILES: c1ccc(-c2ccc(N3c4cc(N(c5ccccc5)c5ccccc5)ccc4B4c5nocc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nocc1N3c1ccc(-c2ccccc2)cc1 ; c1ccc(-c2ccc(N3c4cc(-n5c6ccccc6c6ccccc65)ccc4B4c5nocc5N(c5ccc(-c6ccccc6)cc5)c5cc(-n6c7ccccc7c7ccccc76)cc3c54)cc2)cc1]

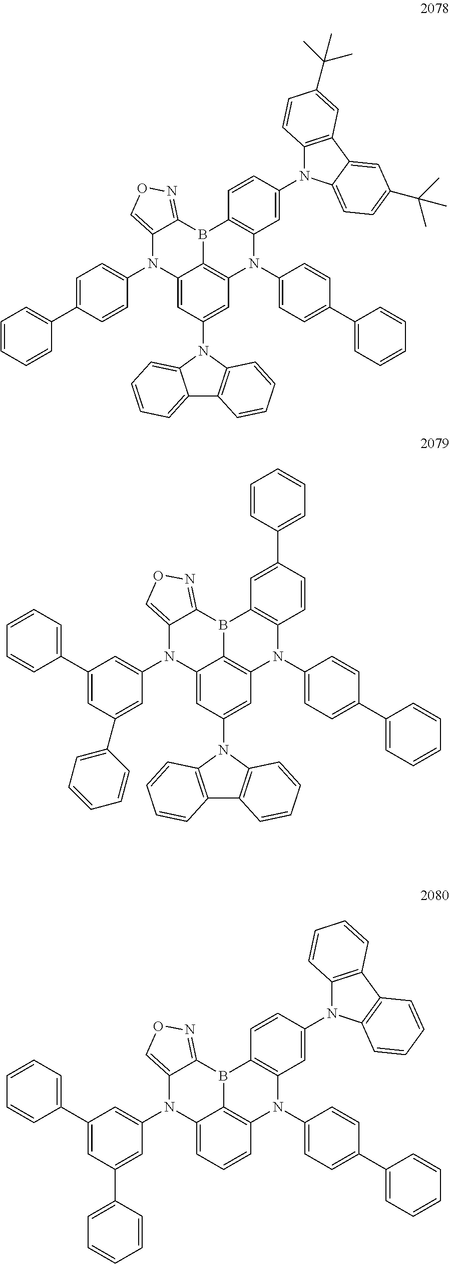 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nocc1N3c1ccc(-c2ccccc2)cc1 ; c1ccc(-c2ccc(N3c4ccc(-c5ccccc5)cc4B4c5nocc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nocc5N(c5cc(-c6ccccc6)cc(-c6ccccc6)c5)c5cccc3c54)cc2)cc1]

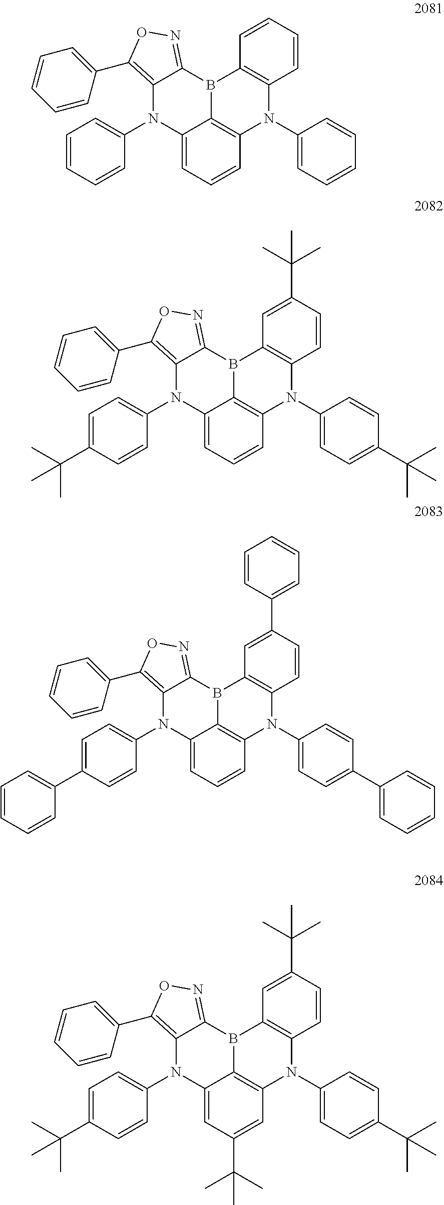

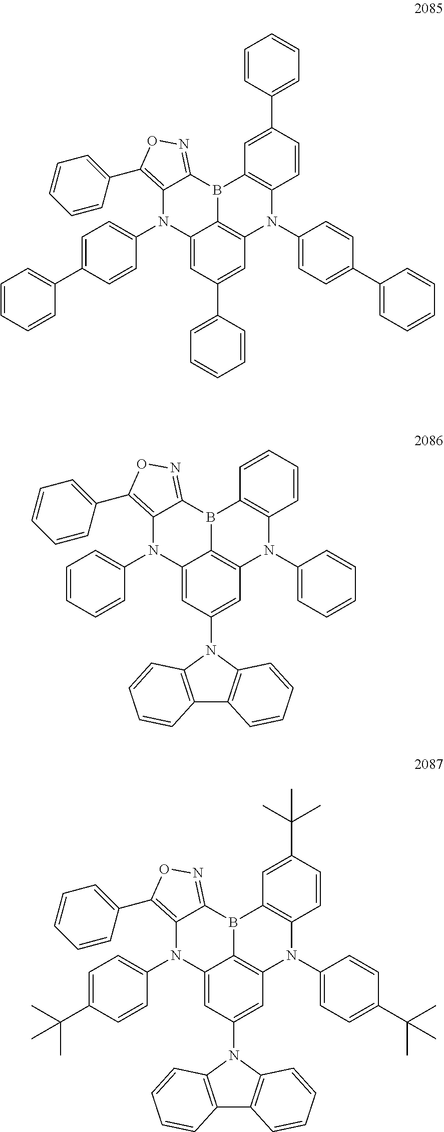 [SMILES: c1ccc(-c2ccc(N3c4ccc(-c5ccccc5)cc4B4c5noc(-c6ccccc6)c5N(c5ccc(-c6ccccc6)cc5)c5cc(-c6ccccc6)cc3c54)cc2)cc1 ; c1ccc(-c2onc3c2N(c2ccccc2)c2cc(-n4c5ccccc5c5ccccc54)cc4c2B3c2ccccc2N4c2ccccc2)cc1 ; CC(C)(C)c1ccc(N2c3ccc(C(C)(C)C)cc3B3c4noc(-c5ccccc5)c4N(c4ccc(C(C)(C)C)cc4)c4cc(-n5c6ccccc6c6ccccc65)cc2c43)cc1]

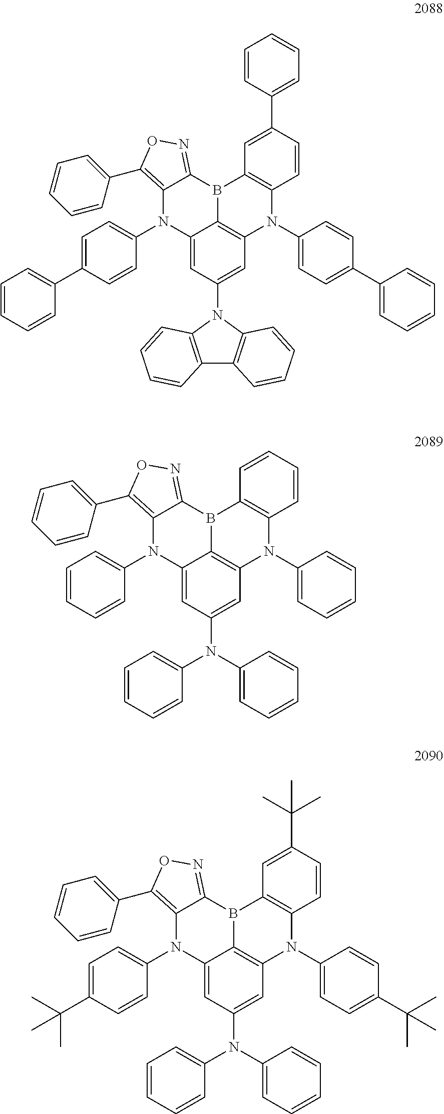 [SMILES: c1ccc(-c2ccc(N3c4ccc(-c5ccccc5)cc4B4c5noc(-c6ccccc6)c5N(c5ccc(-c6ccccc6)cc5)c5cc(-n6c7ccccc7c7ccccc76)cc3c54)cc2)cc1 ; c1ccc(-c2onc3c2N(c2ccccc2)c2cc(N(c4ccccc4)c4ccccc4)cc4c2B3c2ccccc2N4c2ccccc2)cc1 ; CC(C)(C)c1ccc(N2c3ccc(C(C)(C)C)cc3B3c4noc(-c5ccccc5)c4N(c4ccc(C(C)(C)C)cc4)c4cc(N(c5ccccc5)c5ccccc5)cc2c43)cc1]

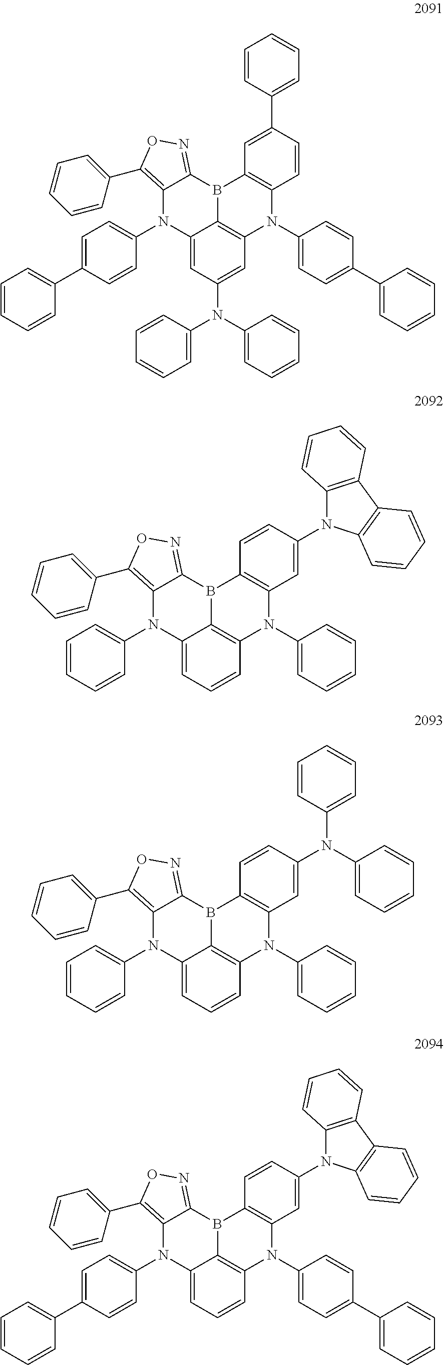 [SMILES: c1ccc(-c2ccc(N3c4ccc(-c5ccccc5)cc4B4c5noc(-c6ccccc6)c5N(c5ccc(-c6ccccc6)cc5)c5cc(N(c6ccccc6)c6ccccc6)cc3c54)cc2)cc1 ; c1ccc(-c2onc3c2N(c2ccccc2)c2cccc4c2B3c2ccc(-n3c5ccccc5c5ccccc53)cc2N4c2ccccc2)cc1 ; c1ccc(-c2onc3c2N(c2ccccc2)c2cccc4c2B3c2ccc(N(c3ccccc3)c3ccccc3)cc2N4c2ccccc2)cc1 ; c1ccc(-c2ccc(N3c4cc(-n5c6ccccc6c6ccccc65)ccc4B4c5noc(-c6ccccc6)c5N(c5ccc(-c6ccccc6)cc5)c5cccc3c54)cc2)cc1]

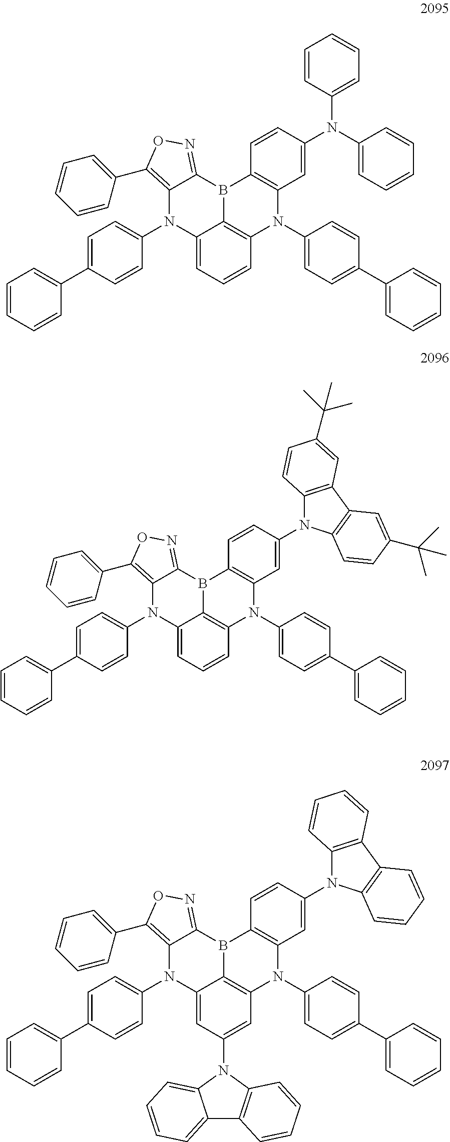 [SMILES: c1ccc(-c2ccc(N3c4cc(N(c5ccccc5)c5ccccc5)ccc4B4c5noc(-c6ccccc6)c5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1noc(-c2ccccc2)c1N3c1ccc(-c2ccccc2)cc1 ; c1ccc(-c2ccc(N3c4cc(-n5c6ccccc6c6ccccc65)ccc4B4c5noc(-c6ccccc6)c5N(c5ccc(-c6ccccc6)cc5)c5cc(-n6c7ccccc7c7ccccc76)cc3c54)cc2)cc1]

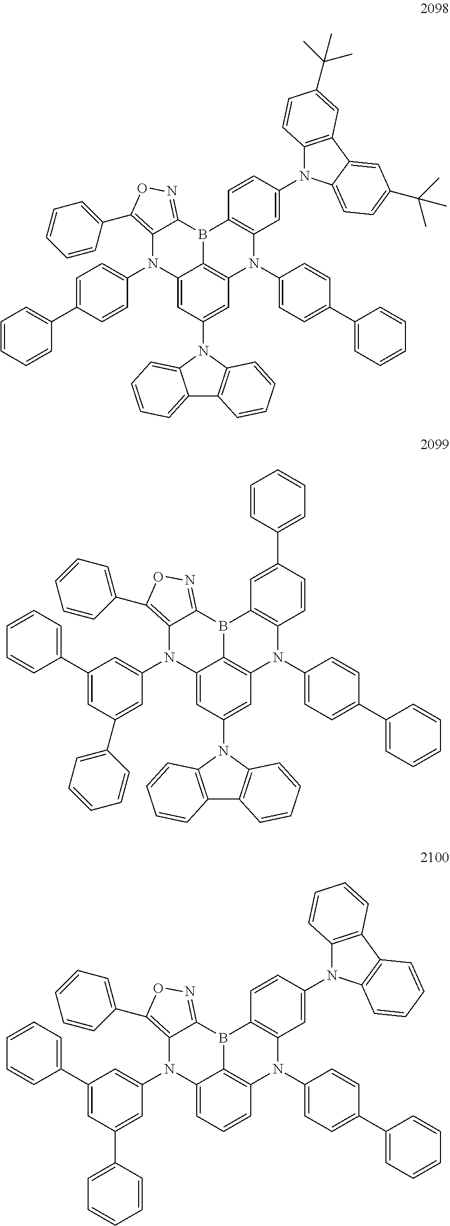 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1noc(-c2ccccc2)c1N3c1ccc(-c2ccccc2)cc1 ; c1ccc(-c2ccc(N3c4ccc(-c5ccccc5)cc4B4c5noc(-c6ccccc6)c5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5noc(-c6ccccc6)c5N(c5cc(-c6ccccc6)cc(-c6ccccc6)c5)c5cccc3c54)cc2)cc1]

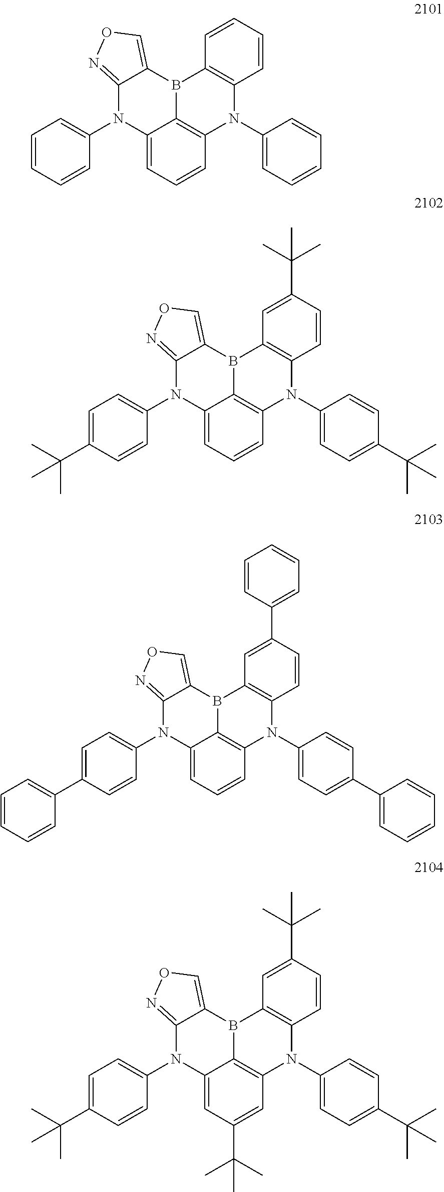

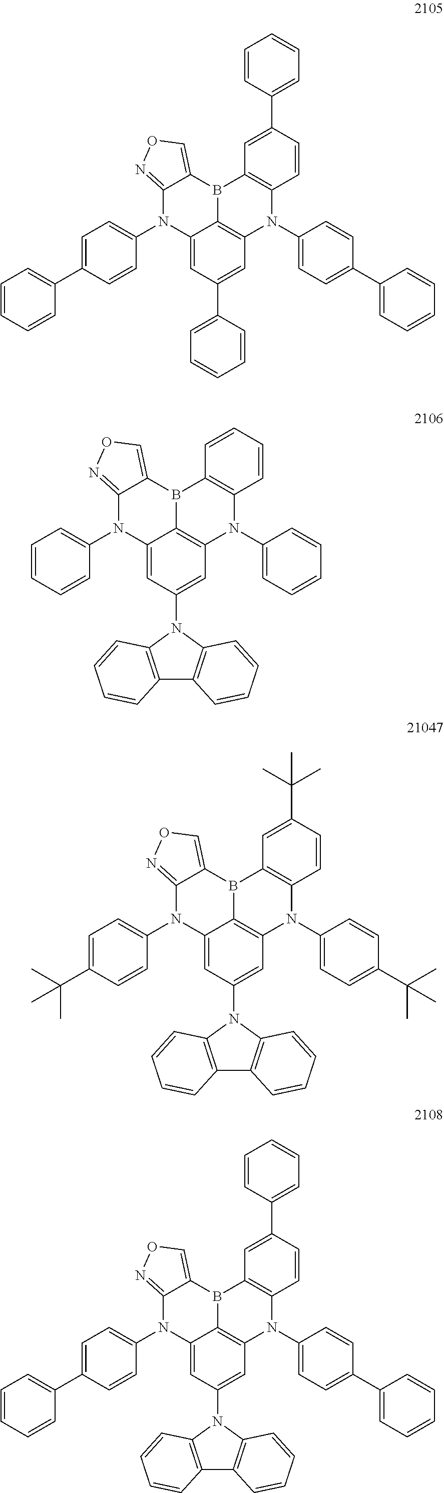 [SMILES: c1ccc(-c2ccc(N3c4ccc(-c5ccccc5)cc4B4c5conc5N(c5ccc(-c6ccccc6)cc5)c5cc(-c6ccccc6)cc3c54)cc2)cc1 ; c1ccc(N2c3ccccc3B3c4conc4N(c4ccccc4)c4cc(-n5c6ccccc6c6ccccc65)cc2c43)cc1 ; CC(C)(C)c1ccc(N2c3ccc(C(C)(C)C)cc3B3c4conc4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4B4c5conc5N(c5ccc(-c6ccccc6)cc5)c5cc(-n6c7ccccc7c7ccccc76)cc3c54)cc2)cc1]

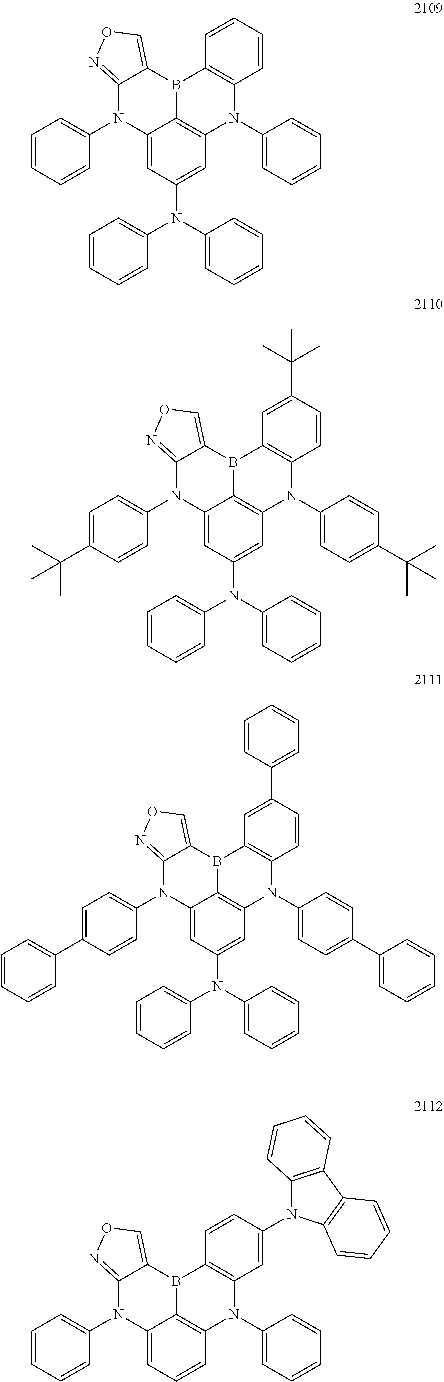 [SMILES: c1ccc(N(c2ccccc2)c2cc3c4c(c2)N(c2ccccc2)c2nocc2B4c2ccccc2N3c2ccccc2)cc1 ; CC(C)(C)c1ccc(N2c3ccc(C(C)(C)C)cc3B3c4conc4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5conc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4conc4N(c4ccccc4)c4cccc2c43)cc1]

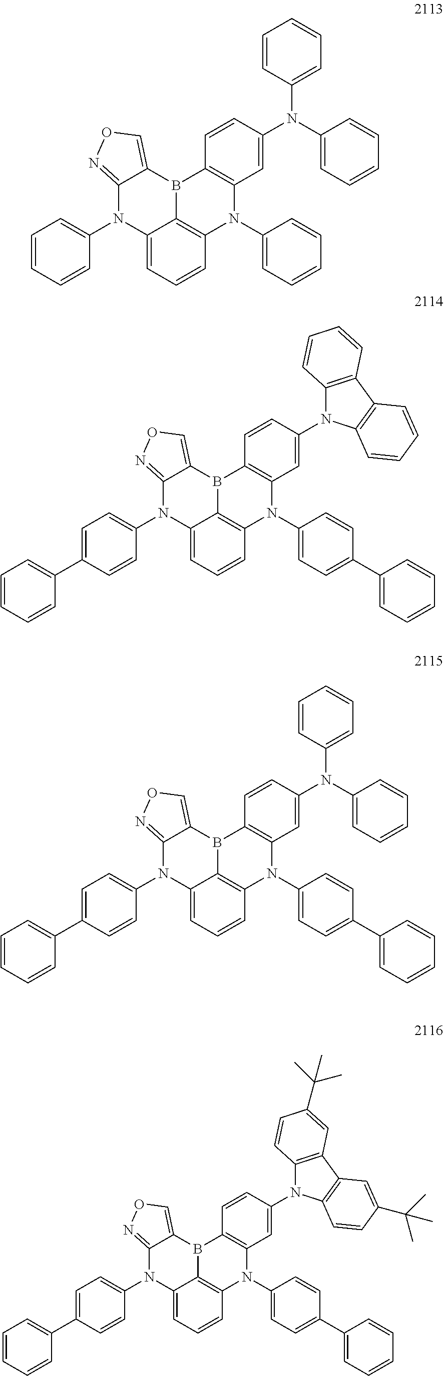 [SMILES: c1ccc(N(c2ccccc2)c2ccc3c(c2)N(c2ccccc2)c2cccc4c2B3c2conc2N4c2ccccc2)cc1 ; c1ccc(-c2ccc(N3c4cc(-n5c6ccccc6c6ccccc65)ccc4B4c5conc5N(c5ccc(-c6ccccc6)cc5)c5cccc3c54)cc2)cc1 ; c1ccc(-c2ccc(N3c4cc(N(c5ccccc5)c5ccccc5)ccc4B4c5conc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1conc1N3c1ccc(-c2ccccc2)cc1]

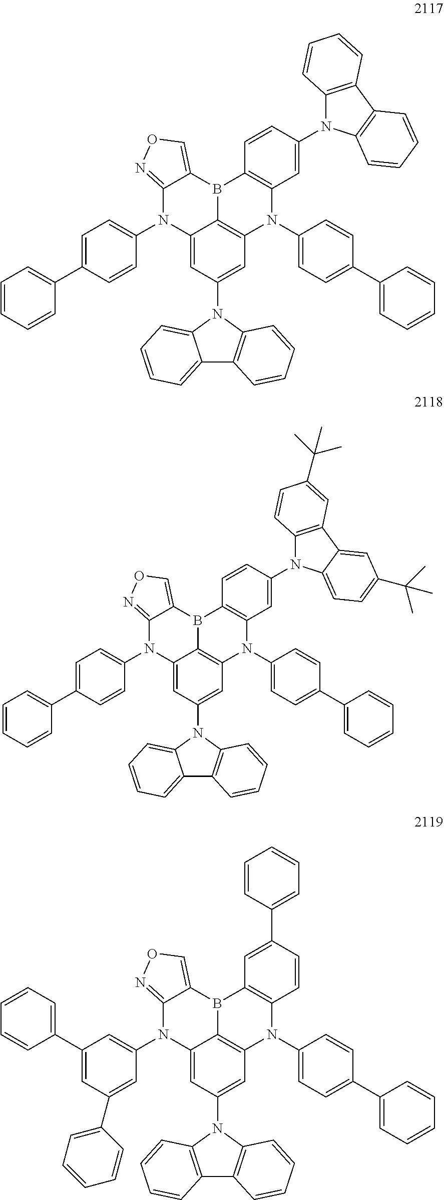 [SMILES: c1ccc(-c2ccc(N3c4cc(-n5c6ccccc6c6ccccc65)ccc4B4c5conc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1conc1N3c1ccc(-c2ccccc2)cc1 ; c1ccc(-c2ccc(N3c4ccc(-c5ccccc5)cc4B4c5conc5N(c5cc(-c6ccccc6)cc(-c6ccccc6)c5)c5cc(-n6c7ccccc7c7ccccc76)cc3c54)cc2)cc1]

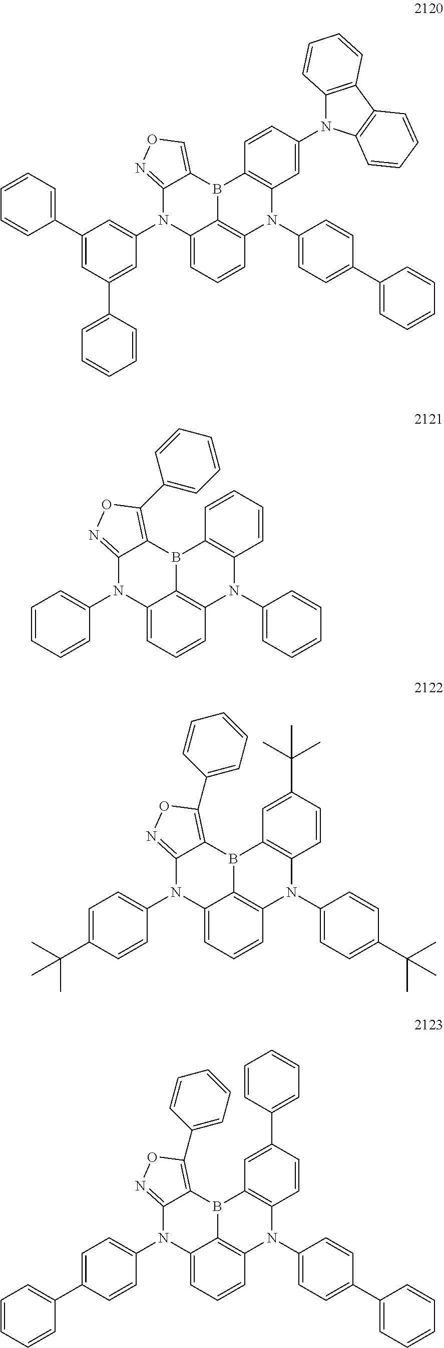 [SMILES: c1ccc(-c2ccc(N3c4cc(-n5c6ccccc6c6ccccc65)ccc4B4c5conc5N(c5cc(-c6ccccc6)cc(-c6ccccc6)c5)c5cccc3c54)cc2)cc1 ; c1ccc(-c2onc3c2B2c4ccccc4N(c4ccccc4)c4cccc(c42)N3c2ccccc2)cc1 ; CC(C)(C)c1ccc(N2c3ccc(C(C)(C)C)cc3B3c4c2cccc4N(c2ccc(C(C)(C)C)cc2)c2noc(-c4ccccc4)c23)cc1 ; c1ccc(-c2ccc(N3c4ccc(-c5ccccc5)cc4B4c5c3cccc5N(c3ccc(-c5ccccc5)cc3)c3noc(-c5ccccc5)c34)cc2)cc1]

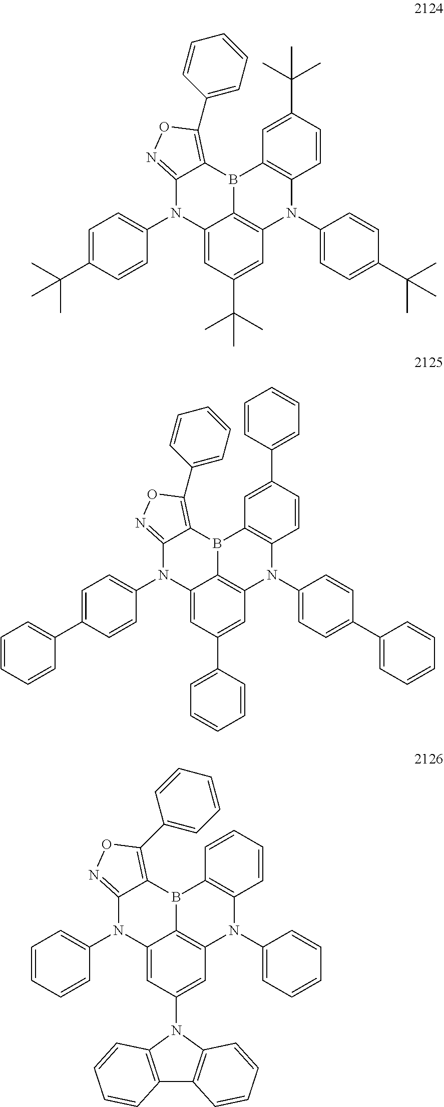

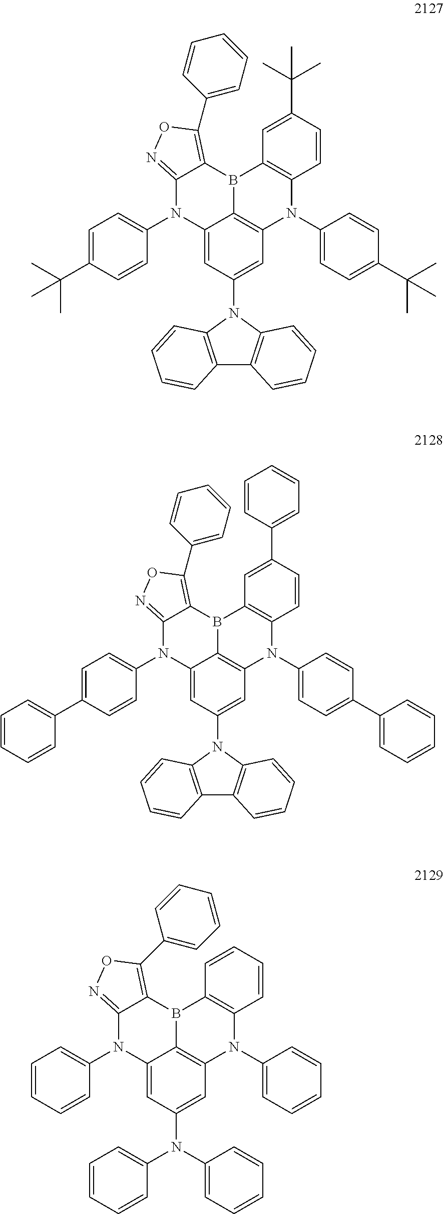 [SMILES: CC(C)(C)c1ccc(N2c3ccc(C(C)(C)C)cc3B3c4c2cc(-n2c5ccccc5c5ccccc52)cc4N(c2ccc(C(C)(C)C)cc2)c2noc(-c4ccccc4)c23)cc1 ; c1ccc(-c2ccc(N3c4ccc(-c5ccccc5)cc4B4c5c3cc(-n3c6ccccc6c6ccccc63)cc5N(c3ccc(-c5ccccc5)cc3)c3noc(-c5ccccc5)c34)cc2)cc1 ; c1ccc(-c2onc3c2B2c4ccccc4N(c4ccccc4)c4cc(N(c5ccccc5)c5ccccc5)cc(c42)N3c2ccccc2)cc1]

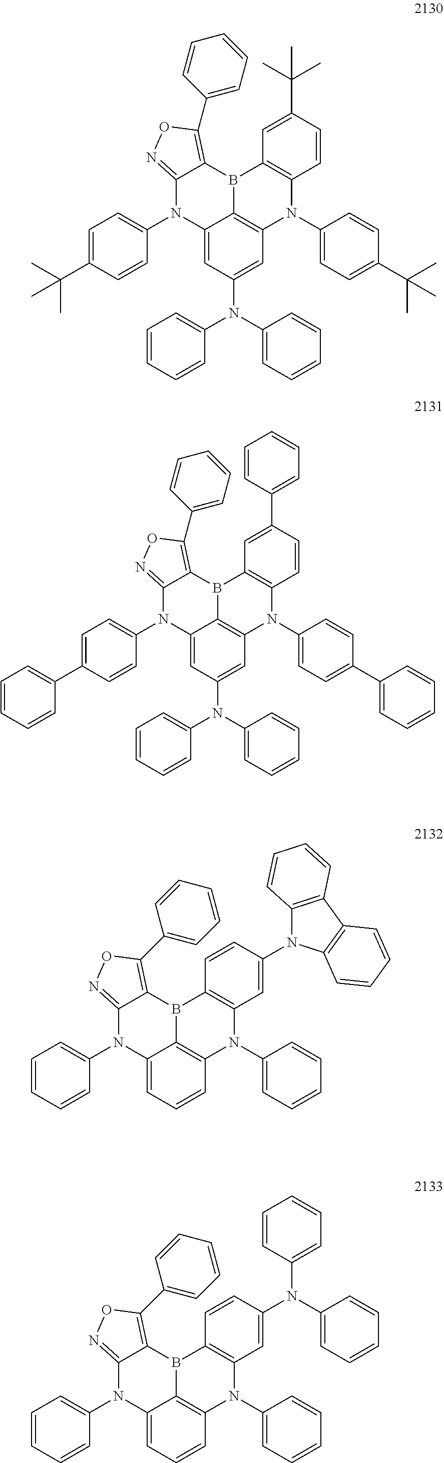 [SMILES: CC(C)(C)c1ccc(N2c3ccc(C(C)(C)C)cc3B3c4c2cc(N(c2ccccc2)c2ccccc2)cc4N(c2ccc(C(C)(C)C)cc2)c2noc(-c4ccccc4)c23)cc1 ; c1ccc(-c2ccc(N3c4ccc(-c5ccccc5)cc4B4c5c3cc(N(c3ccccc3)c3ccccc3)cc5N(c3ccc(-c5ccccc5)cc3)c3noc(-c5ccccc5)c34)cc2)cc1 ; c1ccc(-c2onc3c2B2c4ccc(-n5c6ccccc6c6ccccc65)cc4N(c4ccccc4)c4cccc(c42)N3c2ccccc2)cc1 ; c1ccc(-c2onc3c2B2c4ccc(N(c5ccccc5)c5ccccc5)cc4N(c4ccccc4)c4cccc(c42)N3c2ccccc2)cc1]

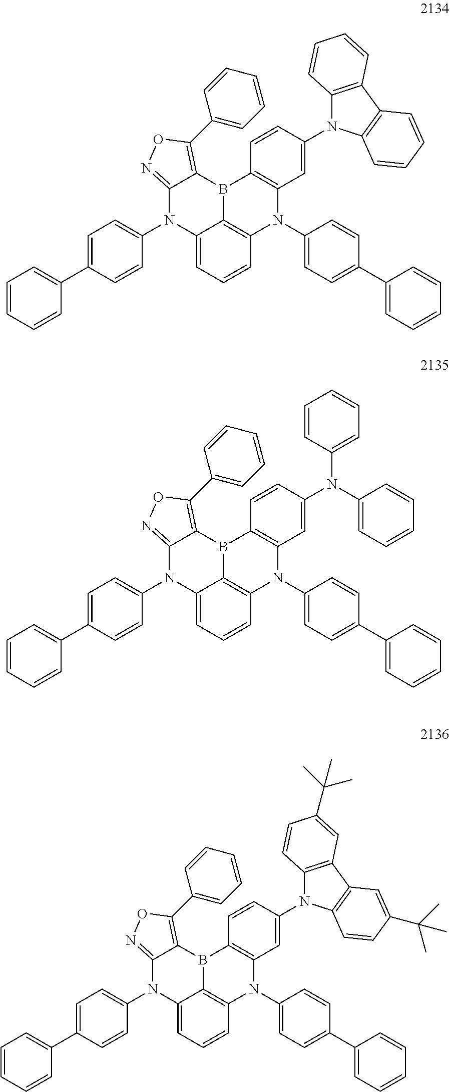 [SMILES: c1ccc(-c2ccc(N3c4cc(-n5c6ccccc6c6ccccc65)ccc4B4c5c3cccc5N(c3ccc(-c5ccccc5)cc3)c3noc(-c5ccccc5)c34)cc2)cc1 ; c1ccc(-c2ccc(N3c4cc(N(c5ccccc5)c5ccccc5)ccc4B4c5c3cccc5N(c3ccc(-c5ccccc5)cc3)c3noc(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1c(noc1-c1ccccc1)N3c1ccc(-c2ccccc2)cc1]

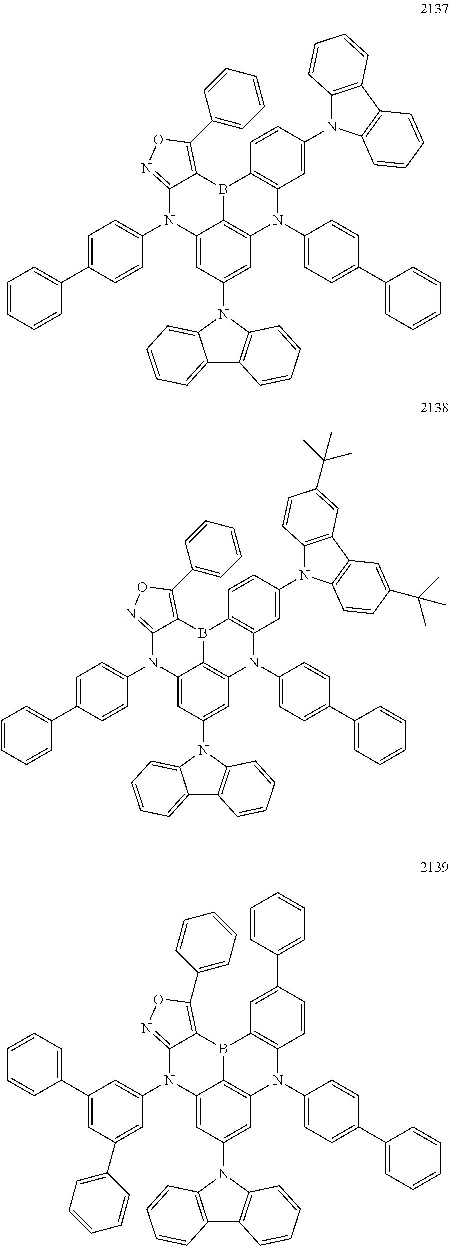 [SMILES: c1ccc(-c2ccc(N3c4cc(-n5c6ccccc6c6ccccc65)ccc4B4c5c3cc(-n3c6ccccc6c6ccccc63)cc5N(c3ccc(-c5ccccc5)cc3)c3noc(-c5ccccc5)c34)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1c(noc1-c1ccccc1)N3c1ccc(-c2ccccc2)cc1 ; c1ccc(-c2ccc(N3c4ccc(-c5ccccc5)cc4B4c5c3cc(-n3c6ccccc6c6ccccc63)cc5N(c3cc(-c5ccccc5)cc(-c5ccccc5)c3)c3noc(-c5ccccc5)c34)cc2)cc1]

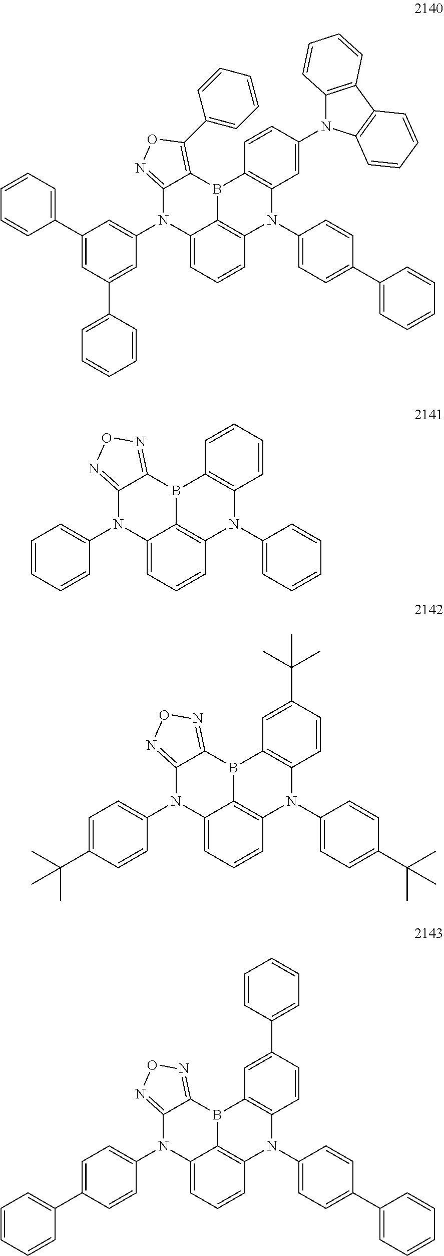

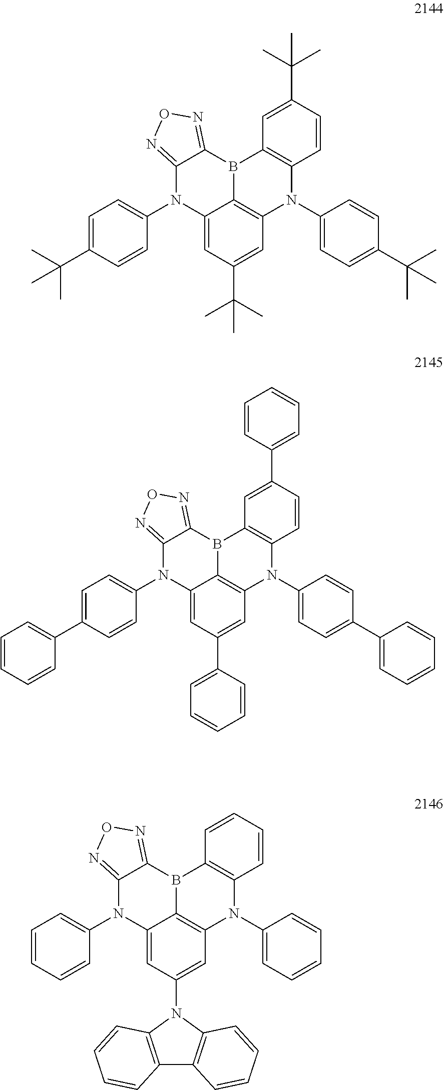 [SMILES: CC(C)(C)c1ccc(N2c3ccc(C(C)(C)C)cc3B3c4nonc4N(c4ccc(C(C)(C)C)cc4)c4cc(C(C)(C)C)cc2c43)cc1 ; c1ccc(-c2ccc(N3c4ccc(-c5ccccc5)cc4B4c5nonc5N(c5ccc(-c6ccccc6)cc5)c5cc(-c6ccccc6)cc3c54)cc2)cc1 ; c1ccc(N2c3ccccc3B3c4nonc4N(c4ccccc4)c4cc(-n5c6ccccc6c6ccccc65)cc2c43)cc1]

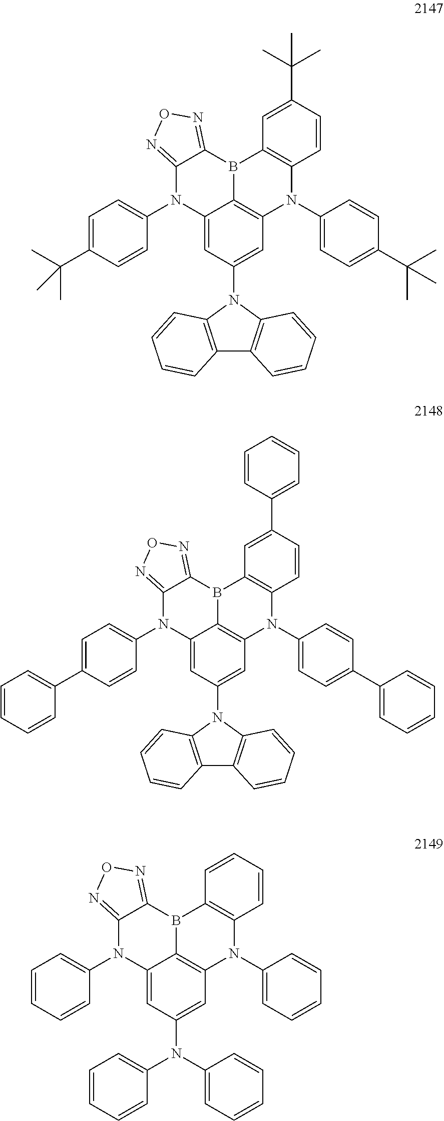 [SMILES: CC(C)(C)c1ccc(N2c3ccc(C(C)(C)C)cc3B3c4nonc4N(c4ccc(C(C)(C)C)cc4)c4cc(-n5c6ccccc6c6ccccc65)cc2c43)cc1 ; c1ccc(-c2ccc(N3c4ccc(-c5ccccc5)cc4B4c5nonc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2nonc2B4c2ccccc2N3c2ccccc2)cc1]

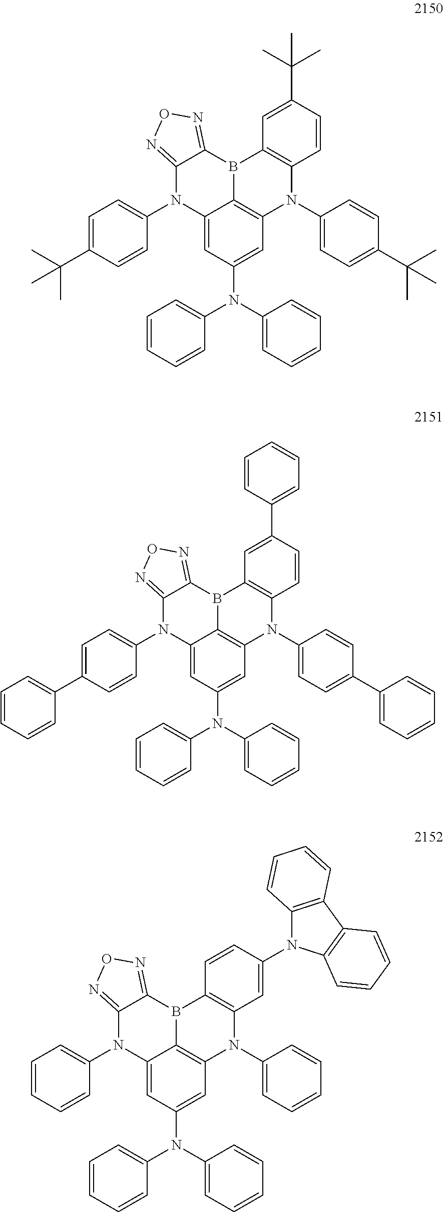 [SMILES: CC(C)(C)c1ccc(N2c3ccc(C(C)(C)C)cc3B3c4nonc4N(c4ccc(C(C)(C)C)cc4)c4cc(N(c5ccccc5)c5ccccc5)cc2c43)cc1 ; c1ccc(-c2ccc(N3c4ccc(-c5ccccc5)cc4B4c5nonc5N(c5ccc(-c6ccccc6)cc5)c5cc(N(c6ccccc6)c6ccccc6)cc3c54)cc2)cc1 ; c1ccc(N(c2ccccc2)c2cc3c4c(c2)N(c2ccccc2)c2nonc2B4c2ccc(-n4c5ccccc5c5ccccc54)cc2N3c2ccccc2)cc1]

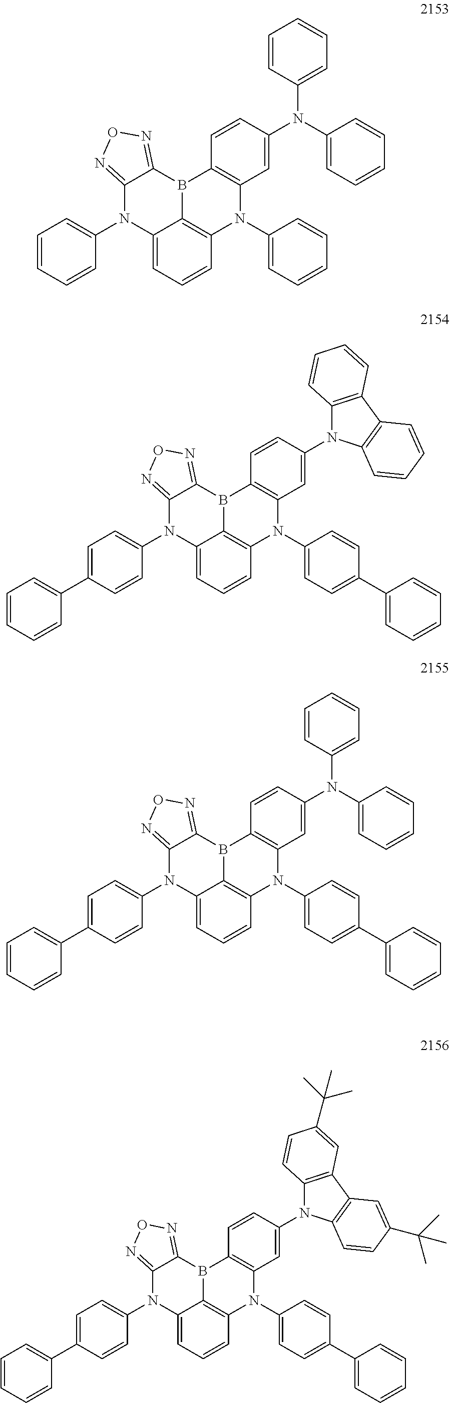 [SMILES: c1ccc(N(c2ccccc2)c2ccc3c(c2)N(c2ccccc2)c2cccc4c2B3c2nonc2N4c2ccccc2)cc1 ; c1ccc(-c2ccc(N3c4cc(-n5c6ccccc6c6ccccc65)ccc4B4c5nonc5N(c5ccc(-c6ccccc6)cc5)c5cccc3c54)cc2)cc1 ; c1ccc(-c2ccc(N3c4cc(N(c5ccccc5)c5ccccc5)ccc4B4c5nonc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nonc1N3c1ccc(-c2ccccc2)cc1]

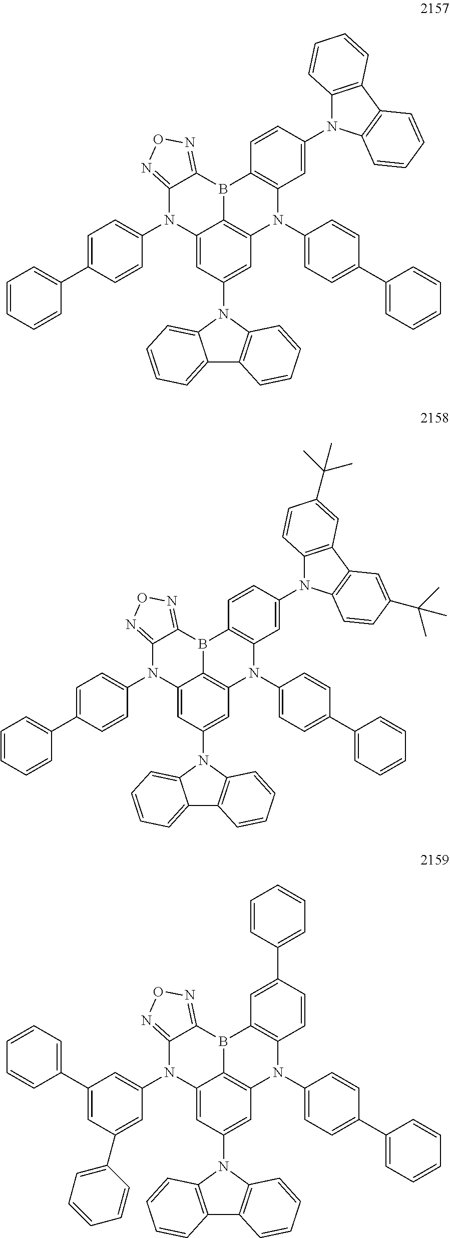 [SMILES: c1ccc(-c2ccc(N3c4cc(-n5c6ccccc6c6ccccc65)ccc4B4c5nonc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nonc1N3c1ccc(-c2ccccc2)cc1 ; c1ccc(-c2ccc(N3c4ccc(-c5ccccc5)cc4B4c5nonc5N(c5cc(-c6ccccc6)cc(-c6ccccc6)c5)c5cc(-n6c7ccccc7c7ccccc76)cc3c54)cc2)cc1]

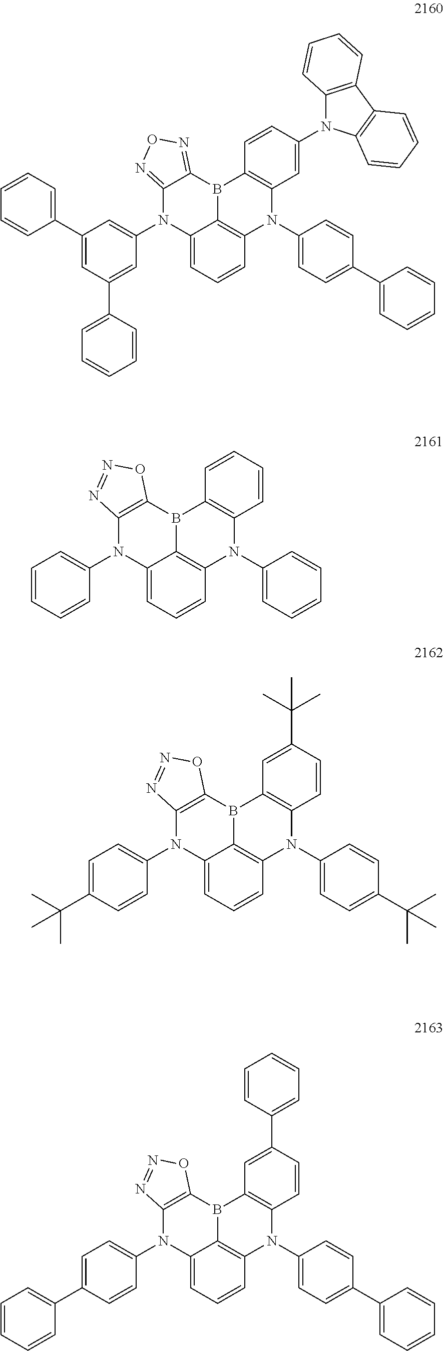 [SMILES: c1ccc(-c2ccc(N3c4cc(-n5c6ccccc6c6ccccc65)ccc4B4c5nonc5N(c5cc(-c6ccccc6)cc(-c6ccccc6)c5)c5cccc3c54)cc2)cc1 ; c1ccc(N2c3ccccc3B3c4onnc4N(c4ccccc4)c4cccc2c43)cc1 ; CC(C)(C)c1ccc(N2c3ccc(C(C)(C)C)cc3B3c4onnc4N(c4ccc(C(C)(C)C)cc4)c4cccc2c43)cc1 ; c1ccc(-c2ccc(N3c4ccc(-c5ccccc5)cc4B4c5onnc5N(c5ccc(-c6ccccc6)cc5)c5cccc3c54)cc2)cc1]

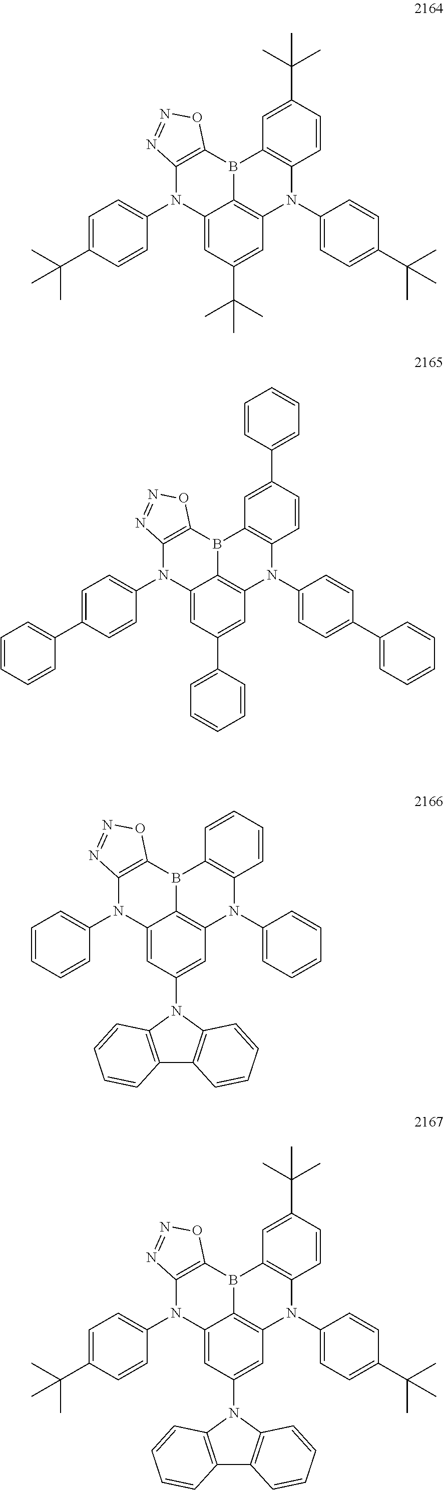

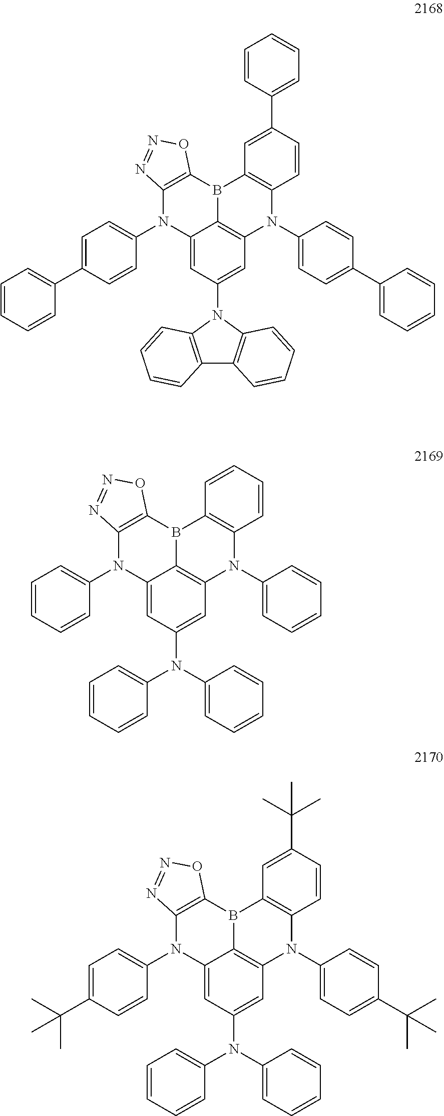 [SMILES: c1ccc(-c2ccc(N3c4ccc(-c5ccccc5)cc4B4c5onnc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2nnoc2B4c2ccccc2N3c2ccccc2)cc1 ; CC(C)(C)c1ccc(N2c3ccc(C(C)(C)C)cc3B3c4onnc4N(c4ccc(C(C)(C)C)cc4)c4cc(N(c5ccccc5)c5ccccc5)cc2c43)cc1]

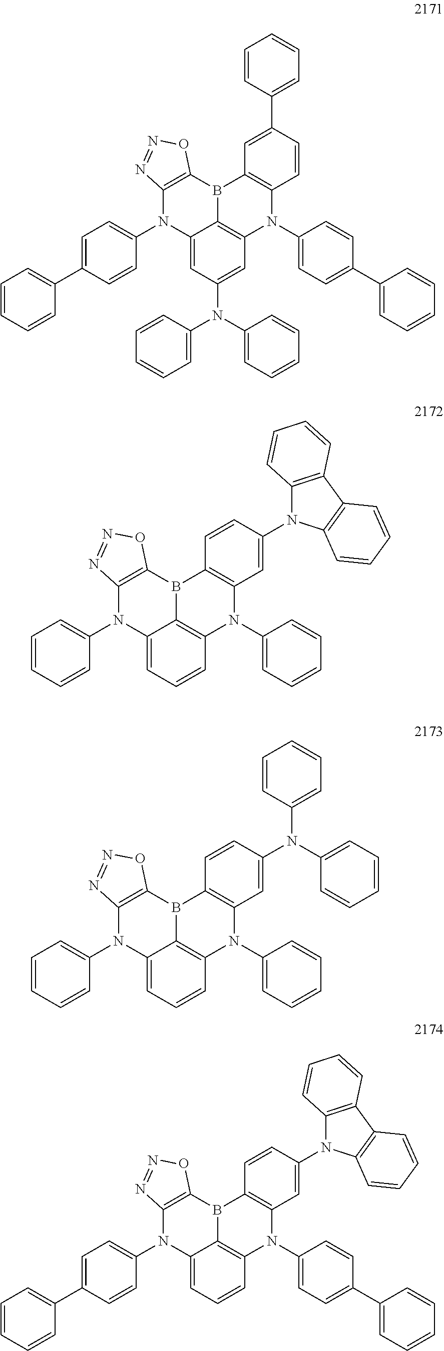 [SMILES: c1ccc(-c2ccc(N3c4ccc(-c5ccccc5)cc4B4c5onnc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4onnc4N(c4ccccc4)c4cccc2c43)cc1 ; c1ccc(N(c2ccccc2)c2ccc3c(c2)N(c2ccccc2)c2cccc4c2B3c2onnc2N4c2ccccc2)cc1 ; c1ccc(-c2ccc(N3c4cc(-n5c6ccccc6c6ccccc65)ccc4B4c5onnc5N(c5ccc(-c6ccccc6)cc5)c5cccc3c54)cc2)cc1]

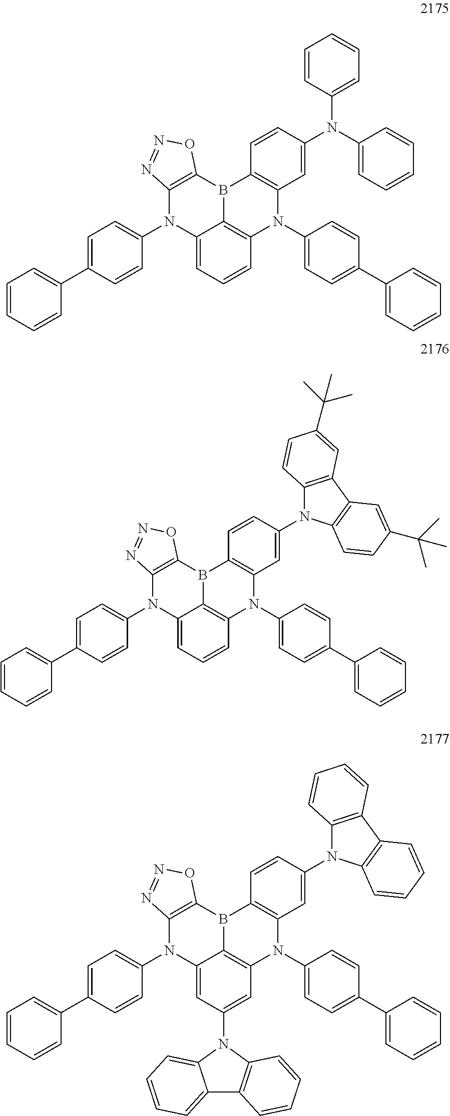 [SMILES: c1ccc(-c2ccc(N3c4cc(N(c5ccccc5)c5ccccc5)ccc4B4c5onnc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1onnc1N3c1ccc(-c2ccccc2)cc1 ; c1ccc(-c2ccc(N3c4cc(-n5c6ccccc6c6ccccc65)ccc4B4c5onnc5N(c5ccc(-c6ccccc6)cc5)c5cc(-n6c7ccccc7c7ccccc76)cc3c54)cc2)cc1]

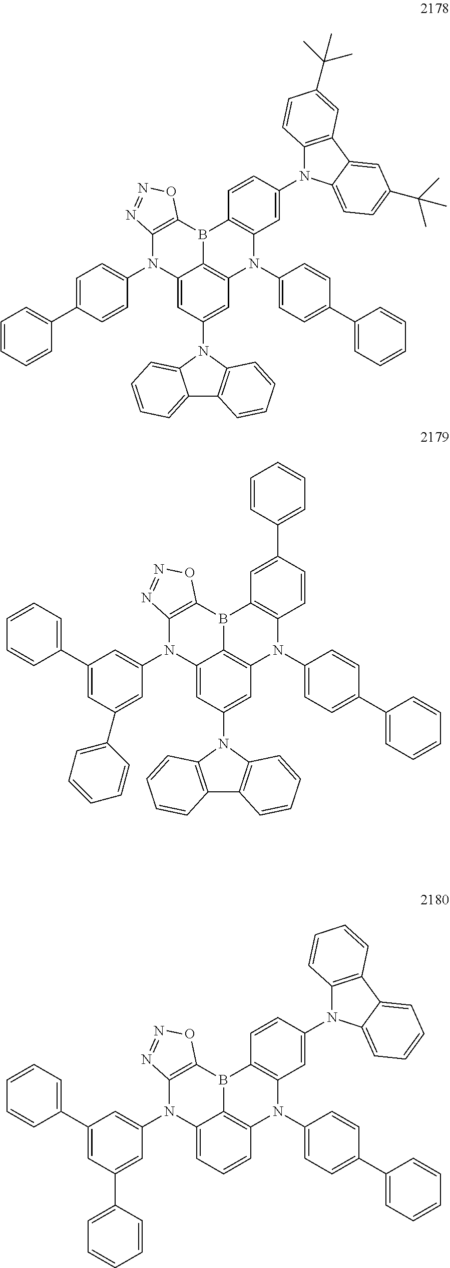 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1onnc1N3c1ccc(-c2ccccc2)cc1 ; c1ccc(-c2ccc(N3c4ccc(-c5ccccc5)cc4B4c5onnc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5onnc5N(c5cc(-c6ccccc6)cc(-c6ccccc6)c5)c5cccc3c54)cc2)cc1]

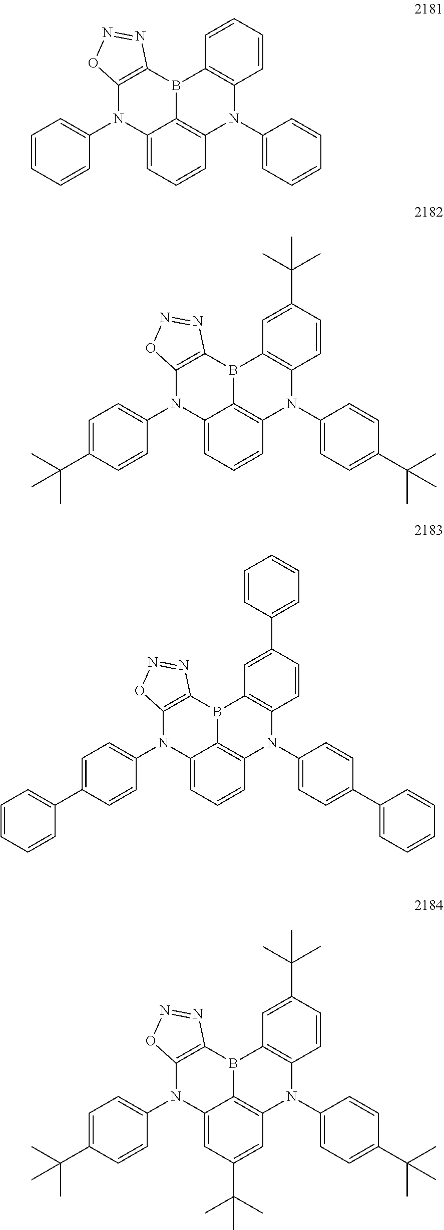

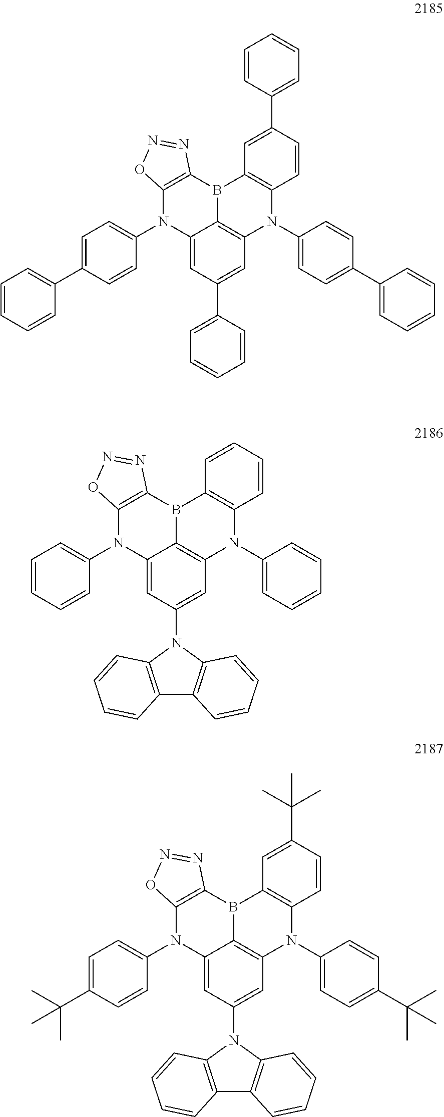 [SMILES: c1ccc(-c2ccc(N3c4ccc(-c5ccccc5)cc4B4c5nnoc5N(c5ccc(-c6ccccc6)cc5)c5cc(-c6ccccc6)cc3c54)cc2)cc1 ; c1ccc(N2c3ccccc3B3c4nnoc4N(c4ccccc4)c4cc(-n5c6ccccc6c6ccccc65)cc2c43)cc1 ; CC(C)(C)c1ccc(N2c3ccc(C(C)(C)C)cc3B3c4nnoc4N(c4ccc(C(C)(C)C)cc4)c4cc(-n5c6ccccc6c6ccccc65)cc2c43)cc1]

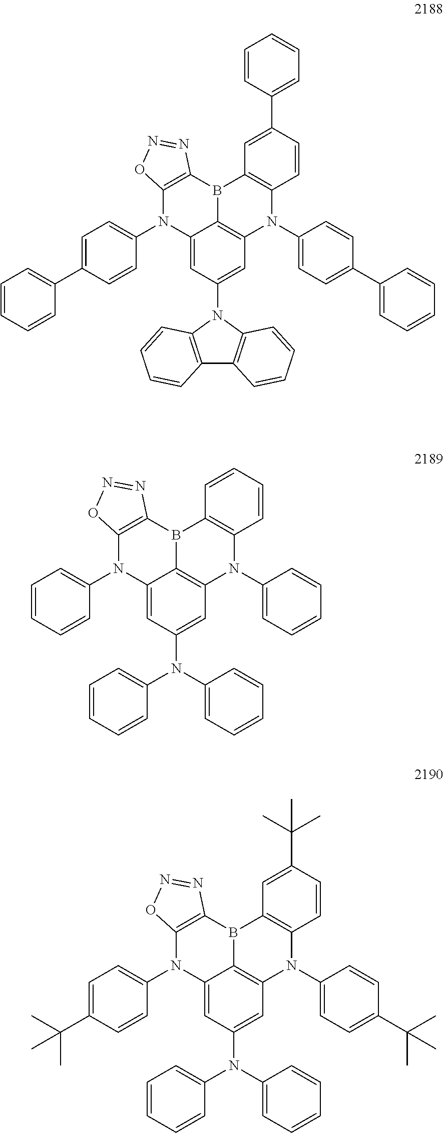 [SMILES: c1ccc(-c2ccc(N3c4ccc(-c5ccccc5)cc4B4c5nnoc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2onnc2B4c2ccccc2N3c2ccccc2)cc1 ; CC(C)(C)c1ccc(N2c3ccc(C(C)(C)C)cc3B3c4nnoc4N(c4ccc(C(C)(C)C)cc4)c4cc(N(c5ccccc5)c5ccccc5)cc2c43)cc1]

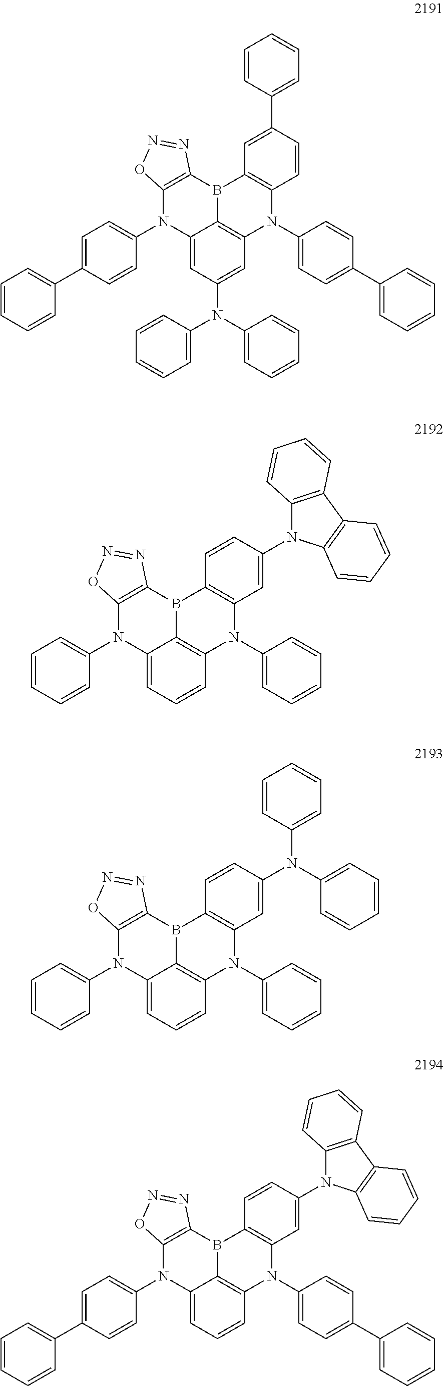 [SMILES: c1ccc(-c2ccc(N3c4ccc(-c5ccccc5)cc4B4c5nnoc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nnoc4N(c4ccccc4)c4cccc2c43)cc1 ; c1ccc(N(c2ccccc2)c2ccc3c(c2)N(c2ccccc2)c2cccc4c2B3c2nnoc2N4c2ccccc2)cc1 ; c1ccc(-c2ccc(N3c4cc(-n5c6ccccc6c6ccccc65)ccc4B4c5nnoc5N(c5ccc(-c6ccccc6)cc5)c5cccc3c54)cc2)cc1]

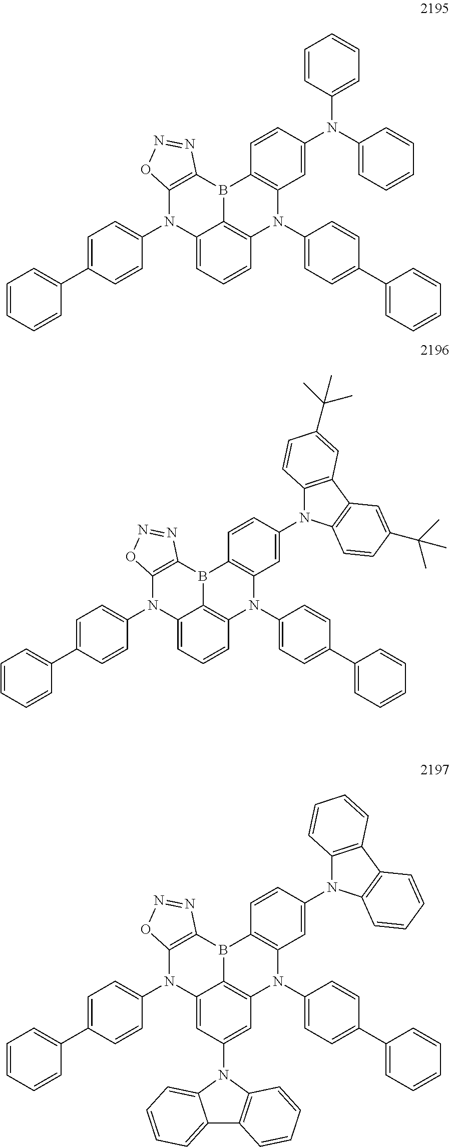 [SMILES: c1ccc(-c2ccc(N3c4cc(N(c5ccccc5)c5ccccc5)ccc4B4c5nnoc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nnoc1N3c1ccc(-c2ccccc2)cc1 ; c1ccc(-c2ccc(N3c4cc(-n5c6ccccc6c6ccccc65)ccc4B4c5nnoc5N(c5ccc(-c6ccccc6)cc5)c5cc(-n6c7ccccc7c7ccccc76)cc3c54)cc2)cc1]

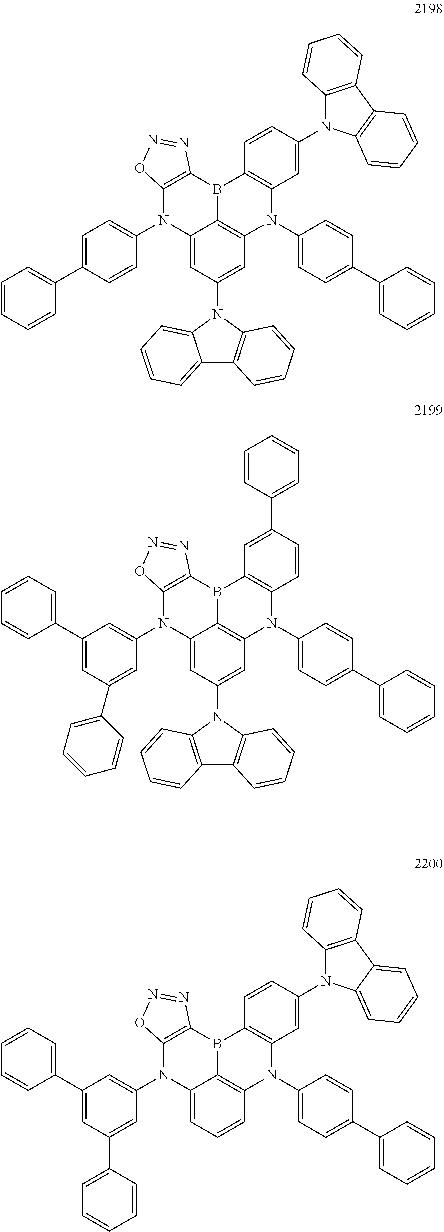 [SMILES: c1ccc(-c2ccc(N3c4cc(-n5c6ccccc6c6ccccc65)ccc4B4c5nnoc5N(c5ccc(-c6ccccc6)cc5)c5cc(-n6c7ccccc7c7ccccc76)cc3c54)cc2)cc1 ; c1ccc(-c2ccc(N3c4ccc(-c5ccccc5)cc4B4c5nnoc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nnoc5N(c5cc(-c6ccccc6)cc(-c6ccccc6)c5)c5cccc3c54)cc2)cc1]

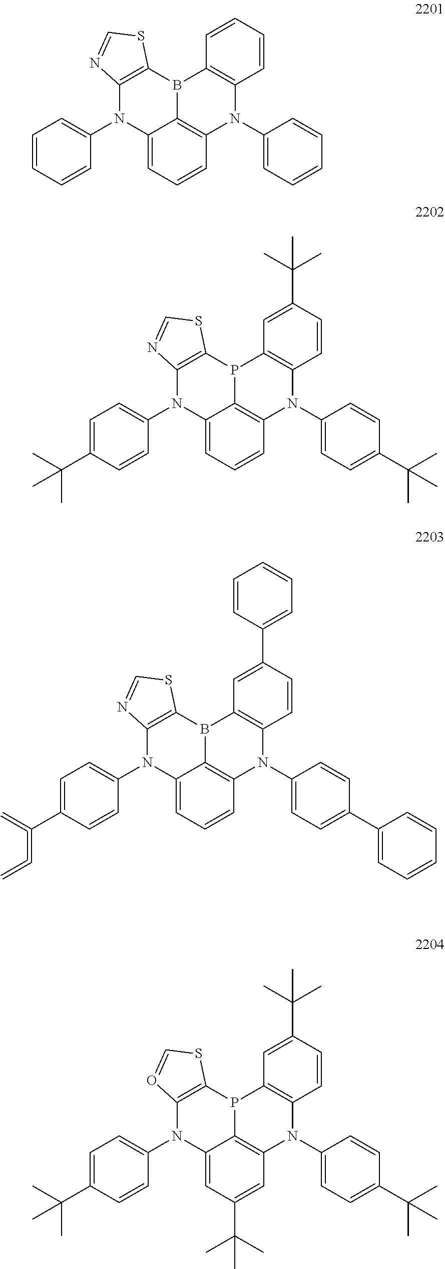

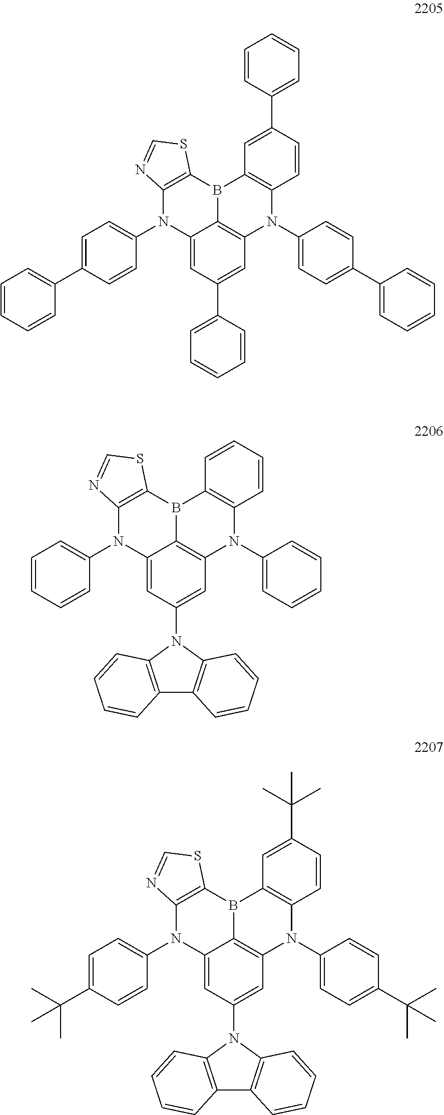 [SMILES: c1ccc(-c2ccc(N3c4ccc(-c5ccccc5)cc4B4c5scnc5N(c5ccc(-c6ccccc6)cc5)c5cc(-c6ccccc6)cc3c54)cc2)cc1 ; c1ccc(N2c3ccccc3B3c4scnc4N(c4ccccc4)c4cc(-n5c6ccccc6c6ccccc65)cc2c43)cc1 ; CC(C)(C)c1ccc(N2c3ccc(C(C)(C)C)cc3B3c4scnc4N(c4ccc(C(C)(C)C)cc4)c4cc(-n5c6ccccc6c6ccccc65)cc2c43)cc1]

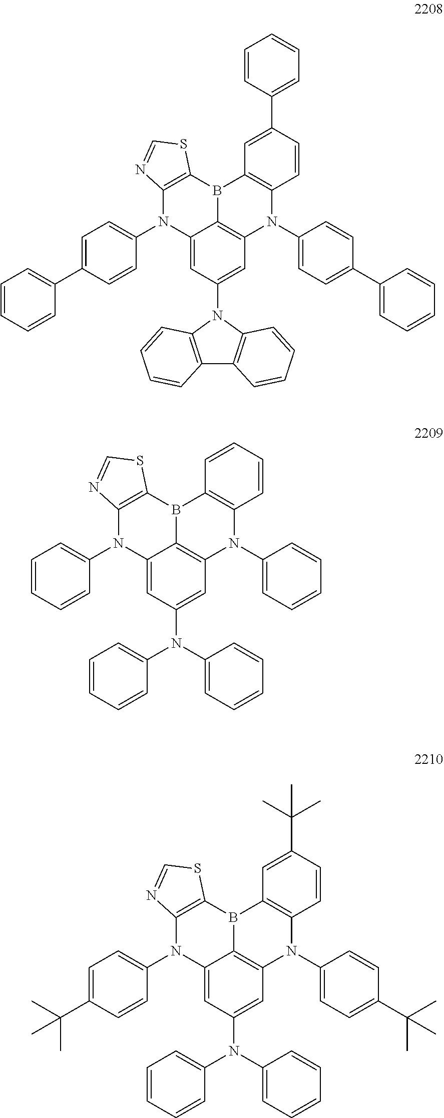 [SMILES: c1ccc(-c2ccc(N3c4ccc(-c5ccccc5)cc4B4c5scnc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2ncsc2B4c2ccccc2N3c2ccccc2)cc1 ; CC(C)(C)c1ccc(N2c3ccc(C(C)(C)C)cc3B3c4scnc4N(c4ccc(C(C)(C)C)cc4)c4cc(N(c5ccccc5)c5ccccc5)cc2c43)cc1]

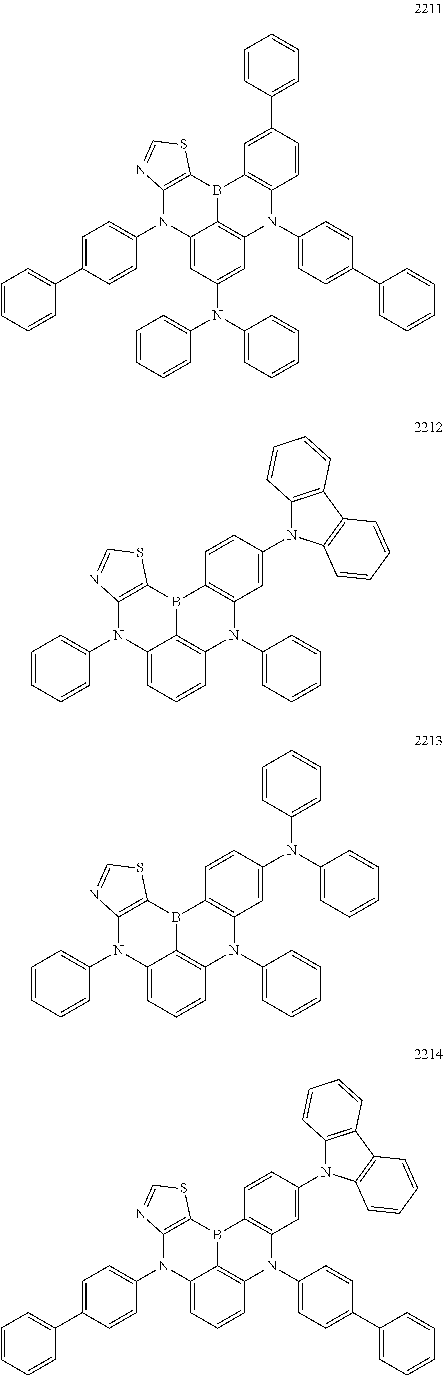 [SMILES: c1ccc(-c2ccc(N3c4ccc(-c5ccccc5)cc4B4c5scnc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4scnc4N(c4ccccc4)c4cccc2c43)cc1 ; c1ccc(N(c2ccccc2)c2ccc3c(c2)N(c2ccccc2)c2cccc4c2B3c2scnc2N4c2ccccc2)cc1 ; c1ccc(-c2ccc(N3c4cc(-n5c6ccccc6c6ccccc65)ccc4B4c5scnc5N(c5ccc(-c6ccccc6)cc5)c5cccc3c54)cc2)cc1]

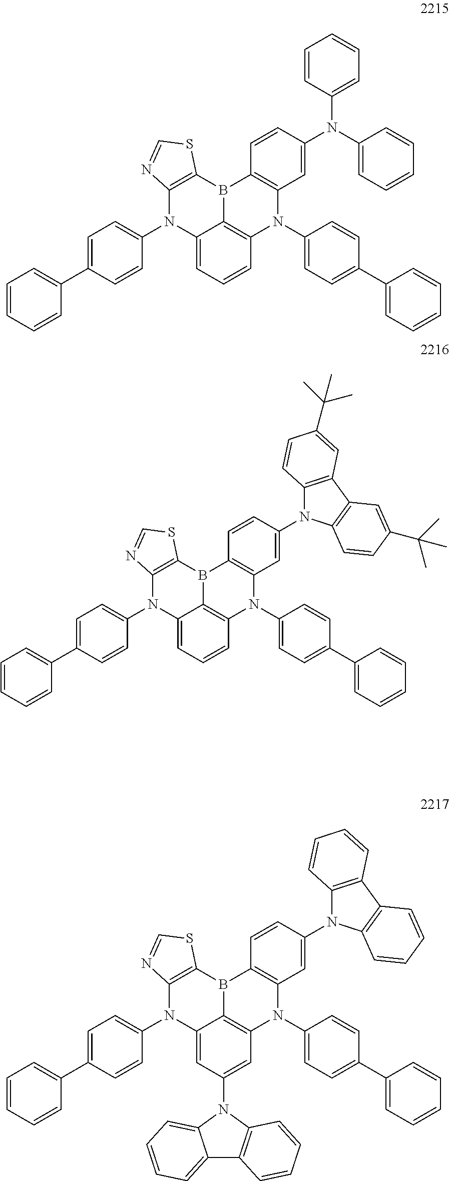 [SMILES: c1ccc(-c2ccc(N3c4cc(N(c5ccccc5)c5ccccc5)ccc4B4c5scnc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1scnc1N3c1ccc(-c2ccccc2)cc1 ; c1ccc(-c2ccc(N3c4cc(-n5c6ccccc6c6ccccc65)ccc4B4c5scnc5N(c5ccc(-c6ccccc6)cc5)c5cc(-n6c7ccccc7c7ccccc76)cc3c54)cc2)cc1]

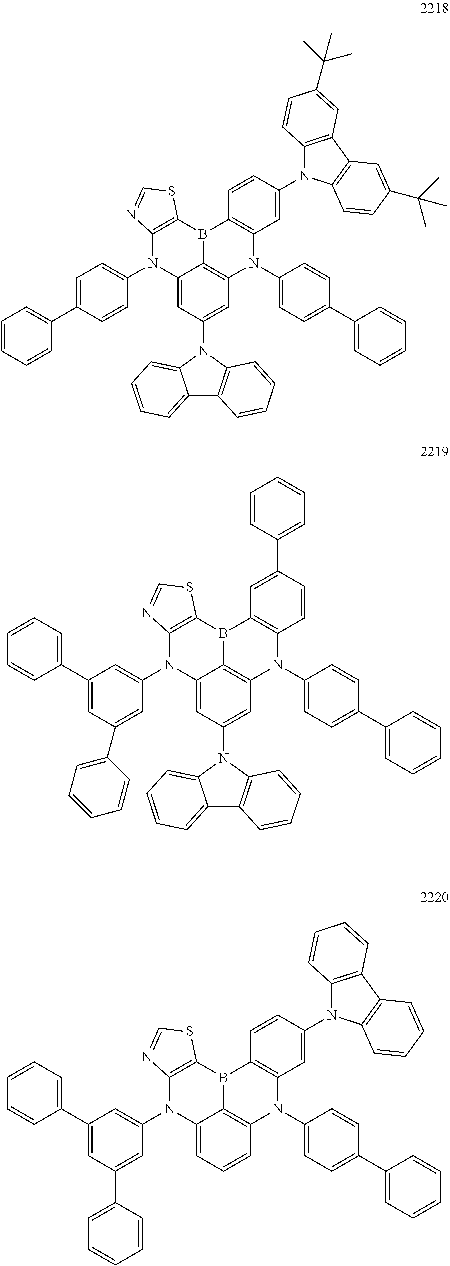 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1scnc1N3c1ccc(-c2ccccc2)cc1 ; c1ccc(-c2ccc(N3c4ccc(-c5ccccc5)cc4B4c5scnc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5scnc5N(c5cc(-c6ccccc6)cc(-c6ccccc6)c5)c5cccc3c54)cc2)cc1]

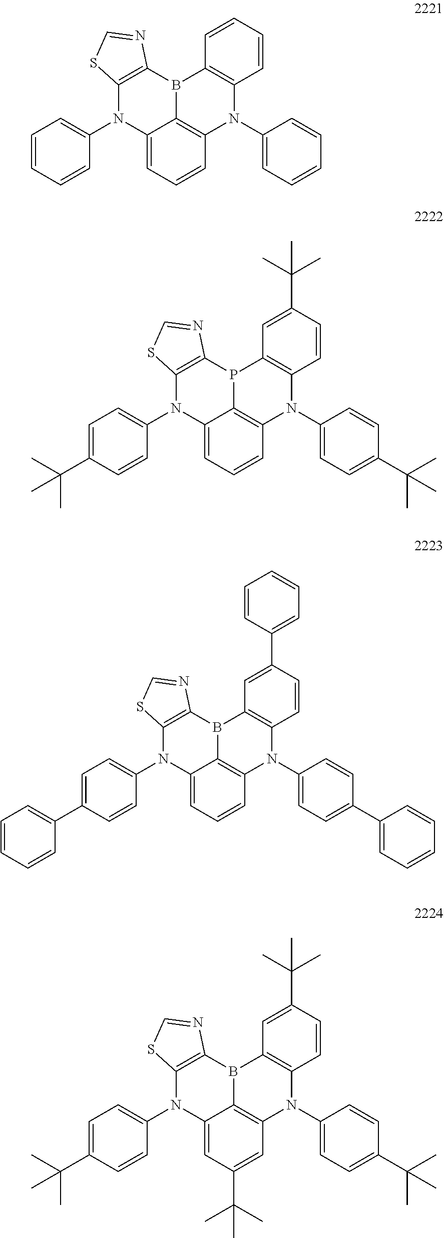

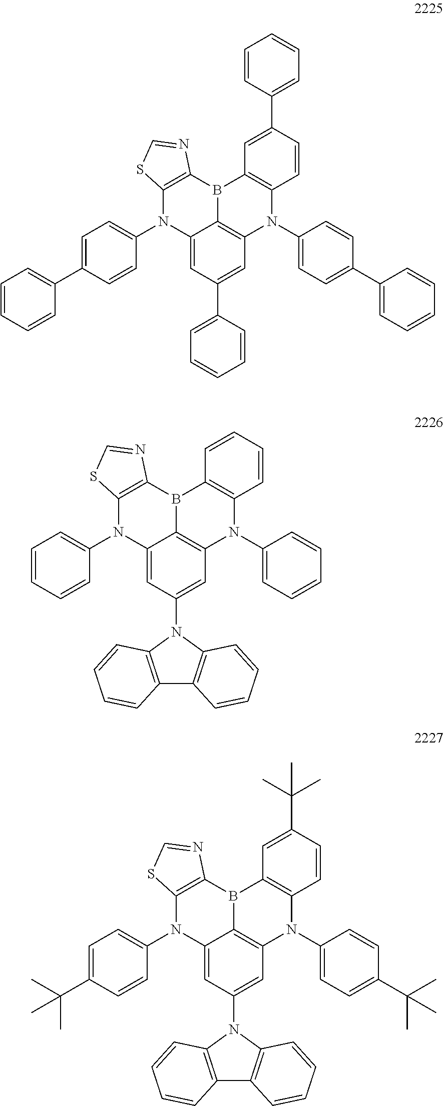 [SMILES: c1ccc(-c2ccc(N3c4ccc(-c5ccccc5)cc4B4c5ncsc5N(c5ccc(-c6ccccc6)cc5)c5cc(-c6ccccc6)cc3c54)cc2)cc1 ; c1ccc(N2c3ccccc3B3c4ncsc4N(c4ccccc4)c4cc(-n5c6ccccc6c6ccccc65)cc2c43)cc1 ; CC(C)(C)c1ccc(N2c3ccc(C(C)(C)C)cc3B3c4ncsc4N(c4ccc(C(C)(C)C)cc4)c4cc(-n5c6ccccc6c6ccccc65)cc2c43)cc1]

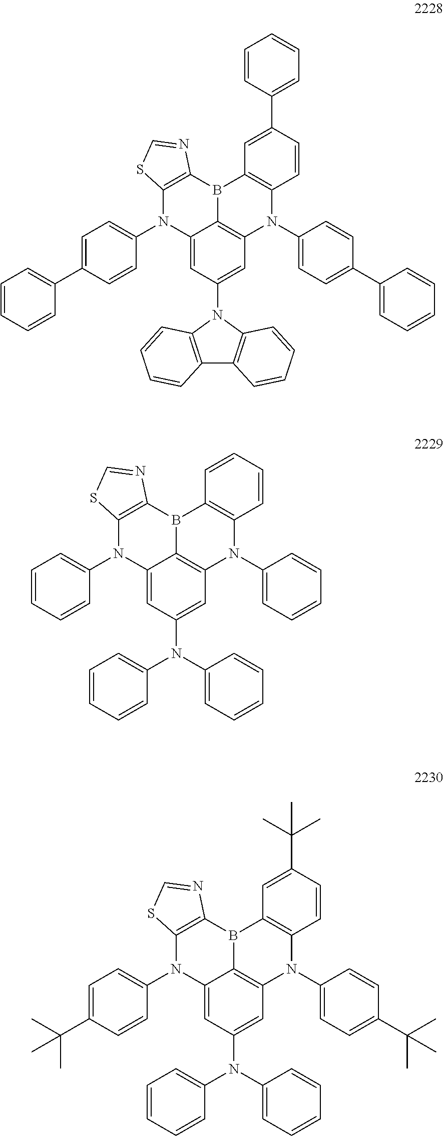 [SMILES: c1ccc(-c2ccc(N3c4ccc(-c5ccccc5)cc4B4c5ncsc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2scnc2B4c2ccccc2N3c2ccccc2)cc1 ; CC(C)(C)c1ccc(N2c3ccc(C(C)(C)C)cc3B3c4ncsc4N(c4ccc(C(C)(C)C)cc4)c4cc(N(c5ccccc5)c5ccccc5)cc2c43)cc1]

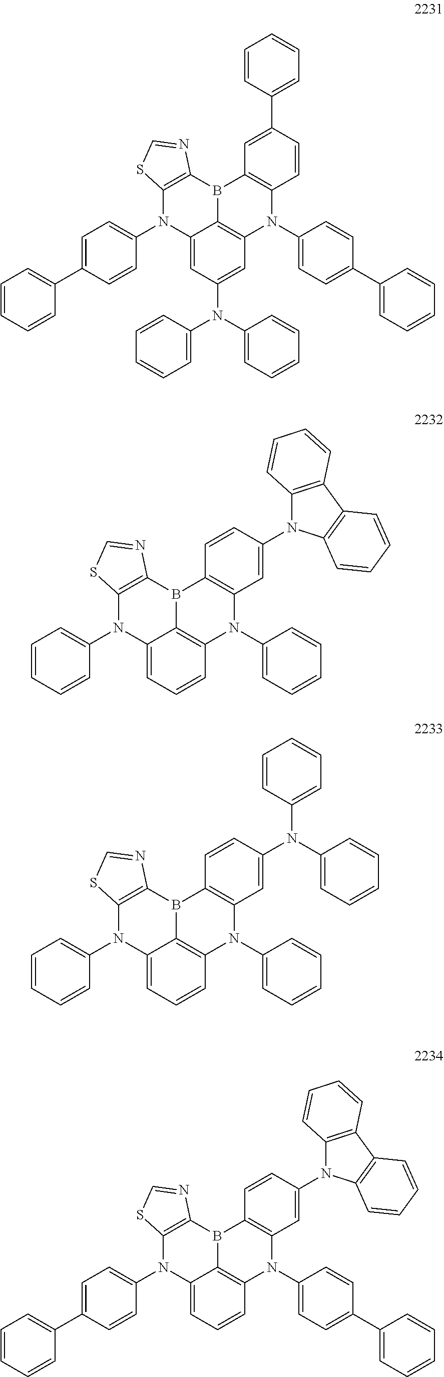 [SMILES: c1ccc(-c2ccc(N3c4ccc(-c5ccccc5)cc4B4c5ncsc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4ncsc4N(c4ccccc4)c4cccc2c43)cc1 ; c1ccc(N(c2ccccc2)c2ccc3c(c2)N(c2ccccc2)c2cccc4c2B3c2ncsc2N4c2ccccc2)cc1 ; c1ccc(-c2ccc(N3c4cc(-n5c6ccccc6c6ccccc65)ccc4B4c5ncsc5N(c5ccc(-c6ccccc6)cc5)c5cccc3c54)cc2)cc1]

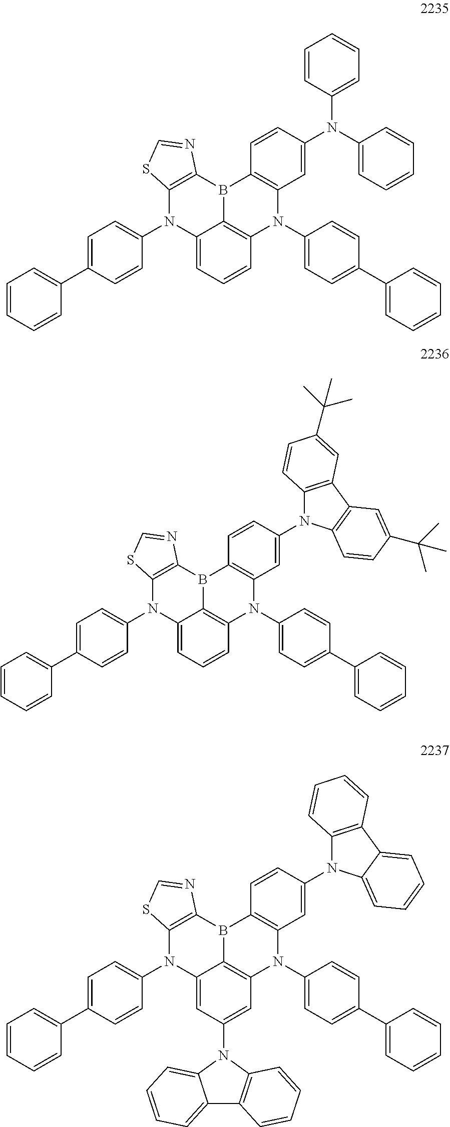 [SMILES: c1ccc(-c2ccc(N3c4cc(N(c5ccccc5)c5ccccc5)ccc4B4c5ncsc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ncsc1N3c1ccc(-c2ccccc2)cc1 ; c1ccc(-c2ccc(N3c4cc(-n5c6ccccc6c6ccccc65)ccc4B4c5ncsc5N(c5ccc(-c6ccccc6)cc5)c5cc(-n6c7ccccc7c7ccccc76)cc3c54)cc2)cc1]

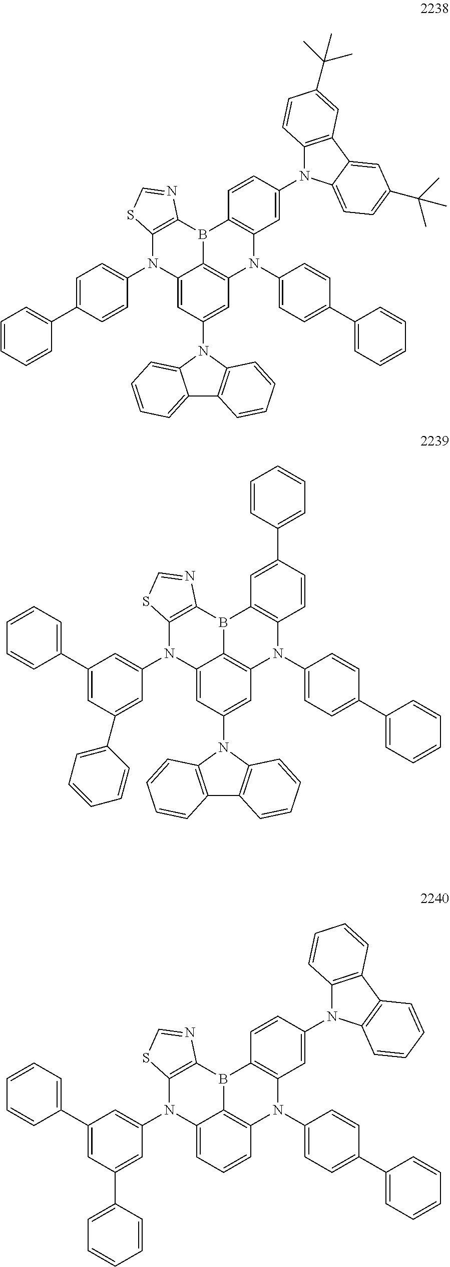 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ncsc1N3c1ccc(-c2ccccc2)cc1 ; c1ccc(-c2ccc(N3c4ccc(-c5ccccc5)cc4B4c5ncsc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5ncsc5N(c5cc(-c6ccccc6)cc(-c6ccccc6)c5)c5cccc3c54)cc2)cc1]

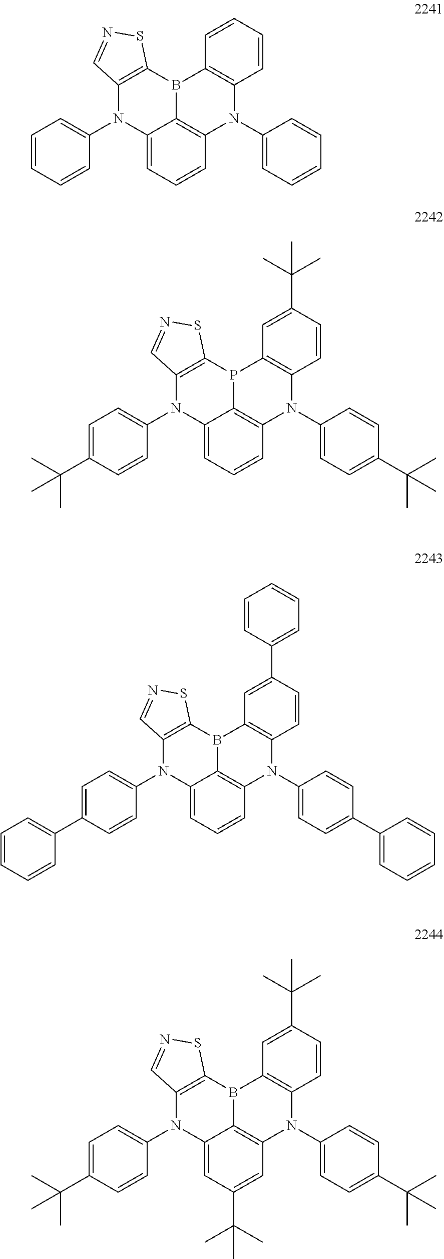

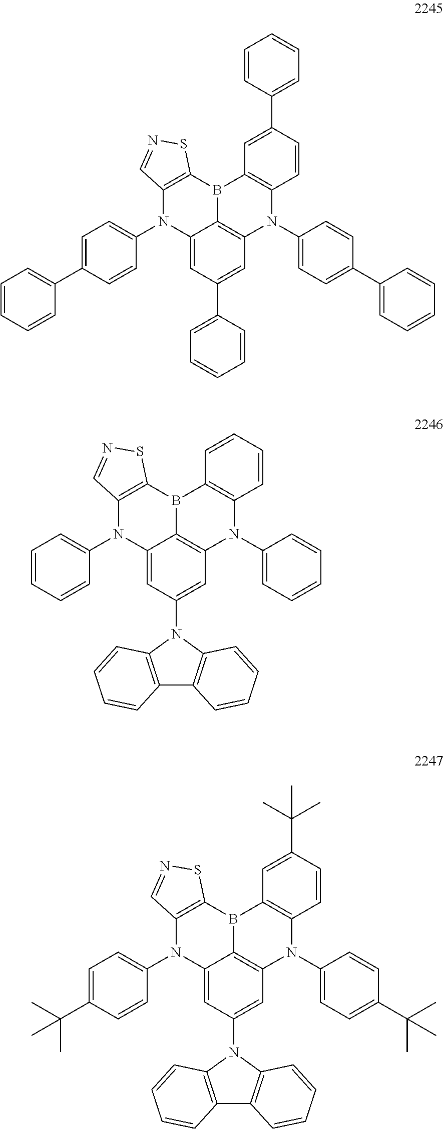 [SMILES: c1ccc(-c2ccc(N3c4ccc(-c5ccccc5)cc4B4c5sncc5N(c5ccc(-c6ccccc6)cc5)c5cc(-c6ccccc6)cc3c54)cc2)cc1 ; c1ccc(N2c3ccccc3B3c4sncc4N(c4ccccc4)c4cc(-n5c6ccccc6c6ccccc65)cc2c43)cc1 ; CC(C)(C)c1ccc(N2c3ccc(C(C)(C)C)cc3B3c4sncc4N(c4ccc(C(C)(C)C)cc4)c4cc(-n5c6ccccc6c6ccccc65)cc2c43)cc1]

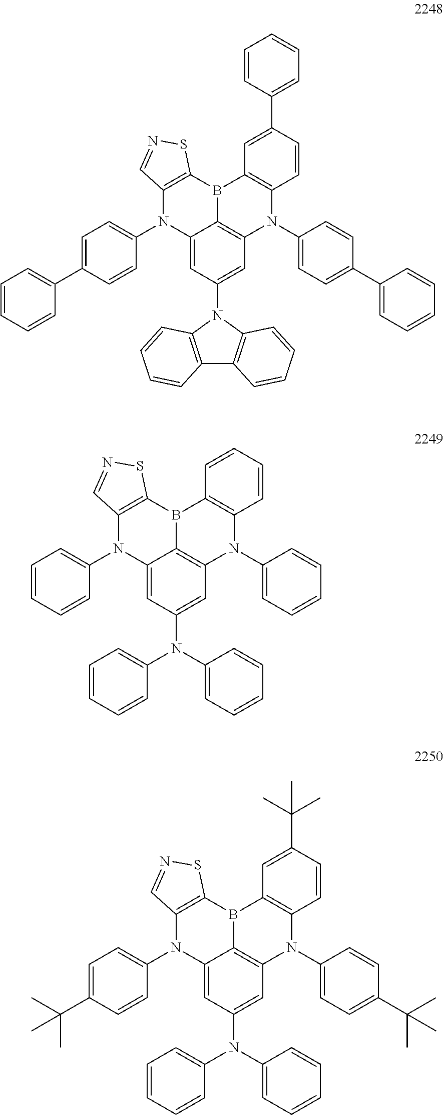 [SMILES: c1ccc(-c2ccc(N3c4ccc(-c5ccccc5)cc4B4c5sncc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2cnsc2B4c2ccccc2N3c2ccccc2)cc1 ; CC(C)(C)c1ccc(N2c3ccc(C(C)(C)C)cc3B3c4sncc4N(c4ccc(C(C)(C)C)cc4)c4cc(N(c5ccccc5)c5ccccc5)cc2c43)cc1]

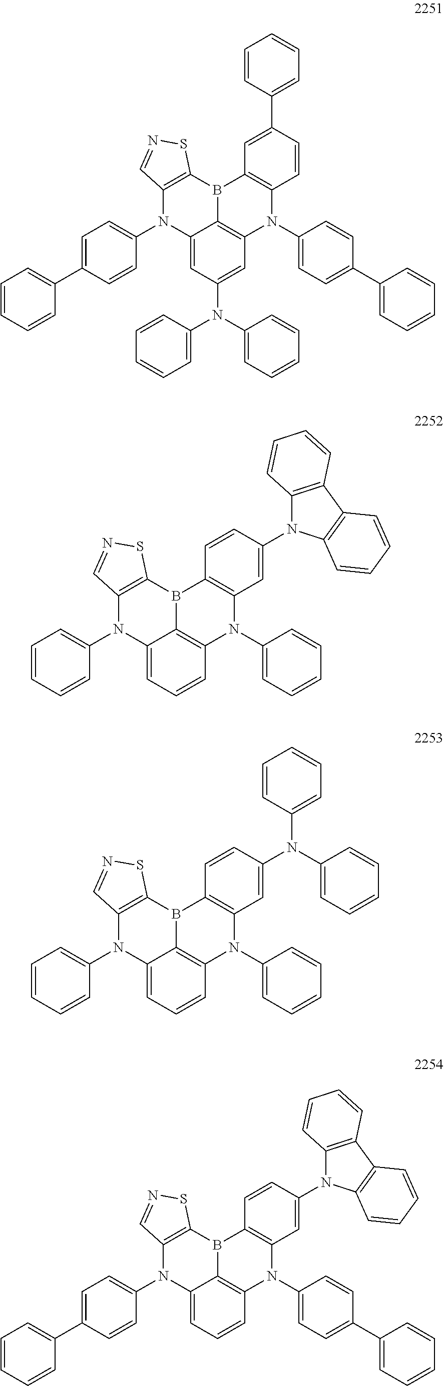 [SMILES: c1ccc(-c2ccc(N3c4ccc(-c5ccccc5)cc4B4c5sncc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4sncc4N(c4ccccc4)c4cccc2c43)cc1 ; c1ccc(N(c2ccccc2)c2ccc3c(c2)N(c2ccccc2)c2cccc4c2B3c2sncc2N4c2ccccc2)cc1 ; c1ccc(-c2ccc(N3c4cc(-n5c6ccccc6c6ccccc65)ccc4B4c5sncc5N(c5ccc(-c6ccccc6)cc5)c5cccc3c54)cc2)cc1]

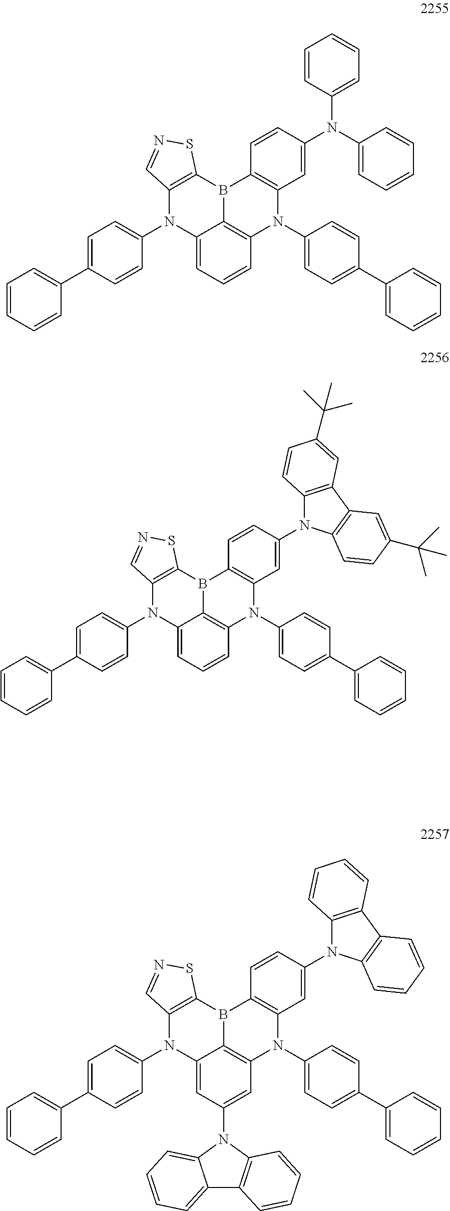 [SMILES: c1ccc(-c2ccc(N3c4cc(N(c5ccccc5)c5ccccc5)ccc4B4c5sncc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1sncc1N3c1ccc(-c2ccccc2)cc1 ; c1ccc(-c2ccc(N3c4cc(-n5c6ccccc6c6ccccc65)ccc4B4c5sncc5N(c5ccc(-c6ccccc6)cc5)c5cc(-n6c7ccccc7c7ccccc76)cc3c54)cc2)cc1]

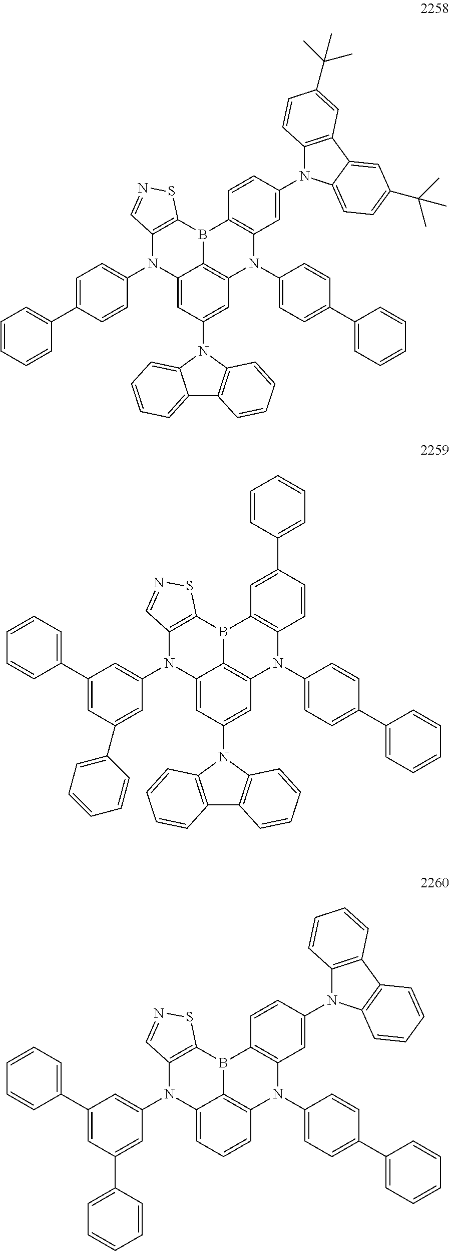 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1sncc1N3c1ccc(-c2ccccc2)cc1 ; c1ccc(-c2ccc(N3c4ccc(-c5ccccc5)cc4B4c5sncc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5sncc5N(c5cc(-c6ccccc6)cc(-c6ccccc6)c5)c5cccc3c54)cc2)cc1]

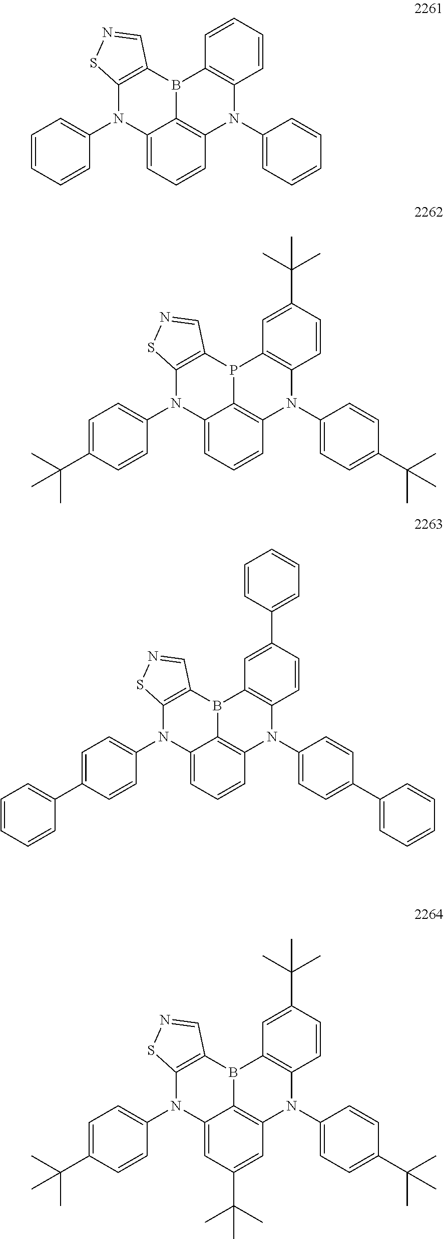

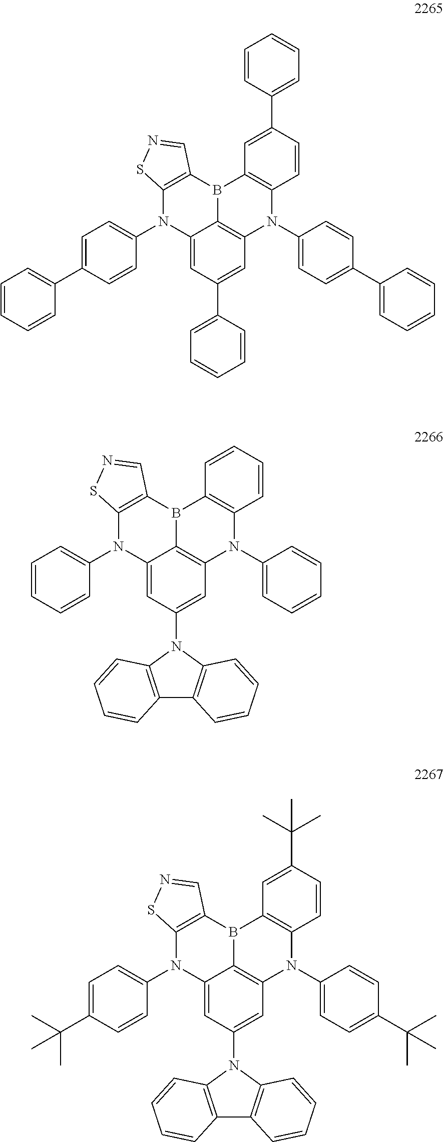 [SMILES: c1ccc(-c2ccc(N3c4ccc(-c5ccccc5)cc4B4c5cnsc5N(c5ccc(-c6ccccc6)cc5)c5cc(-c6ccccc6)cc3c54)cc2)cc1 ; c1ccc(N2c3ccccc3B3c4cnsc4N(c4ccccc4)c4cc(-n5c6ccccc6c6ccccc65)cc2c43)cc1 ; CC(C)(C)c1ccc(N2c3ccc(C(C)(C)C)cc3B3c4cnsc4N(c4ccc(C(C)(C)C)cc4)c4cc(-n5c6ccccc6c6ccccc65)cc2c43)cc1]

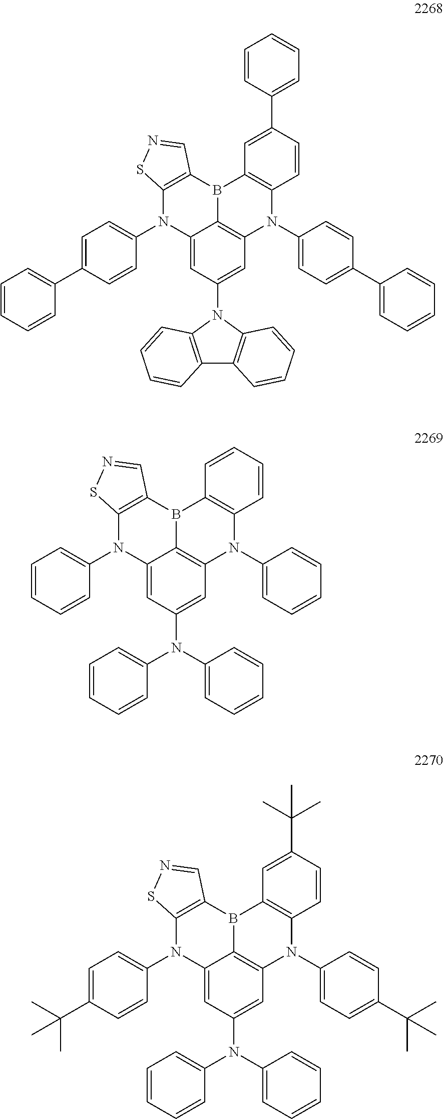 [SMILES: c1ccc(-c2ccc(N3c4ccc(-c5ccccc5)cc4B4c5cnsc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2sncc2B4c2ccccc2N3c2ccccc2)cc1 ; CC(C)(C)c1ccc(N2c3ccc(C(C)(C)C)cc3B3c4cnsc4N(c4ccc(C(C)(C)C)cc4)c4cc(N(c5ccccc5)c5ccccc5)cc2c43)cc1]

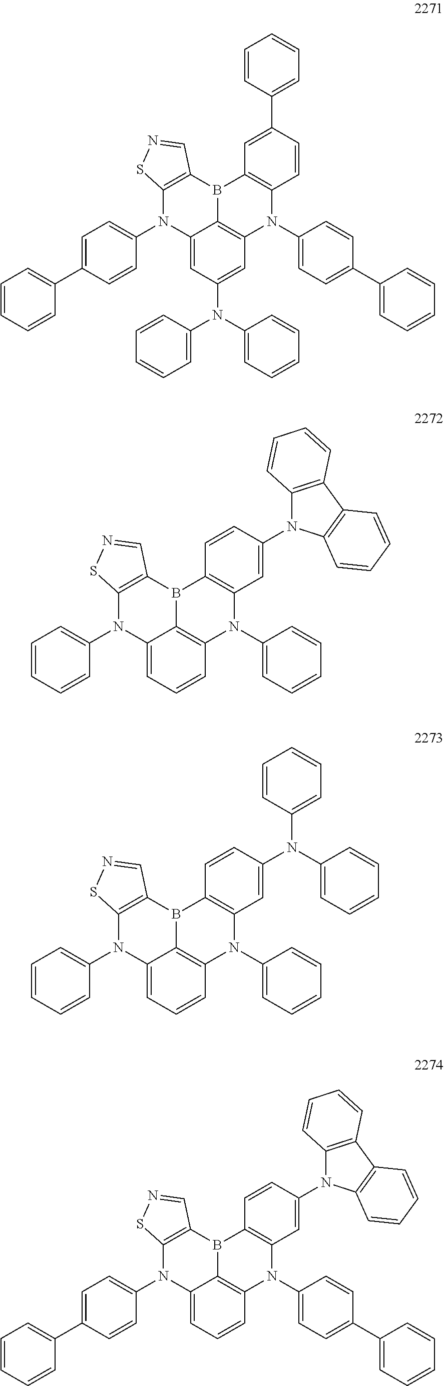 [SMILES: c1ccc(-c2ccc(N3c4ccc(-c5ccccc5)cc4B4c5cnsc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4cnsc4N(c4ccccc4)c4cccc2c43)cc1 ; c1ccc(N(c2ccccc2)c2ccc3c(c2)N(c2ccccc2)c2cccc4c2B3c2cnsc2N4c2ccccc2)cc1 ; c1ccc(-c2ccc(N3c4cc(-n5c6ccccc6c6ccccc65)ccc4B4c5cnsc5N(c5ccc(-c6ccccc6)cc5)c5cccc3c54)cc2)cc1]

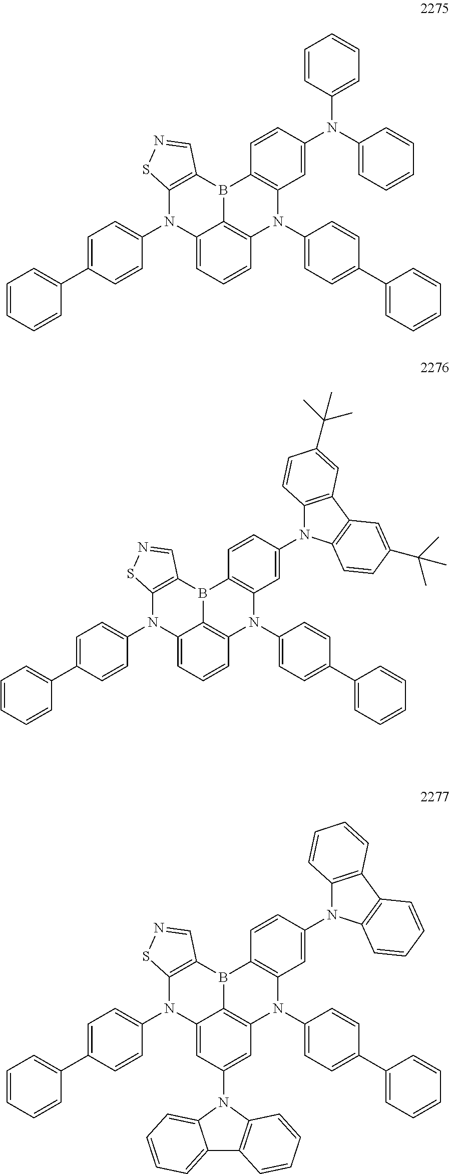 [SMILES: c1ccc(-c2ccc(N3c4cc(N(c5ccccc5)c5ccccc5)ccc4B4c5cnsc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1cnsc1N3c1ccc(-c2ccccc2)cc1 ; c1ccc(-c2ccc(N3c4cc(-n5c6ccccc6c6ccccc65)ccc4B4c5cnsc5N(c5ccc(-c6ccccc6)cc5)c5cc(-n6c7ccccc7c7ccccc76)cc3c54)cc2)cc1]

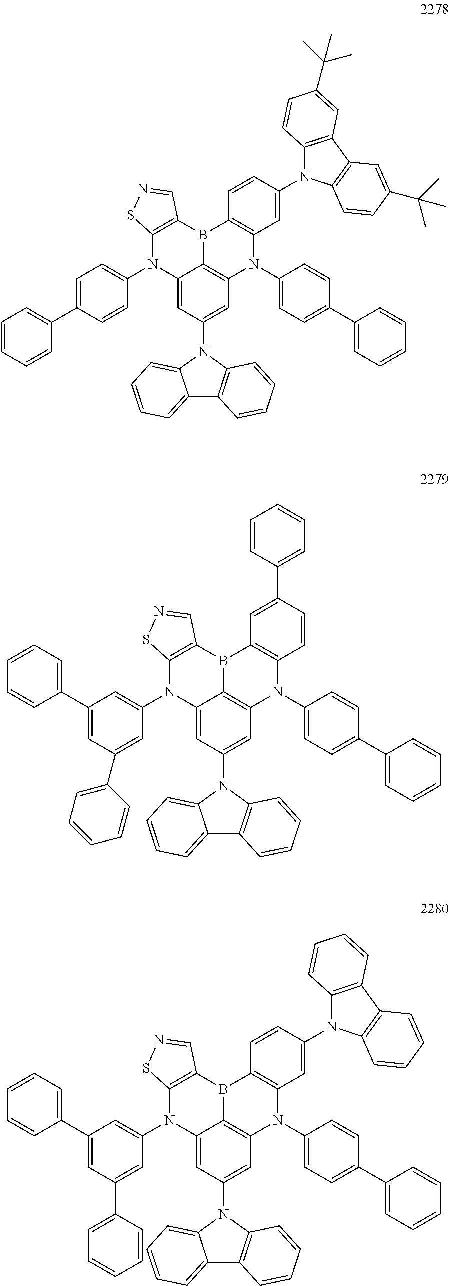 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1cnsc1N3c1ccc(-c2ccccc2)cc1 ; c1ccc(-c2ccc(N3c4ccc(-c5ccccc5)cc4B4c5cnsc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5cnsc5N(c5cc(-c6ccccc6)cc(-c6ccccc6)c5)c5cc(-n6c7ccccc7c7ccccc76)cc3c54)cc2)cc1]

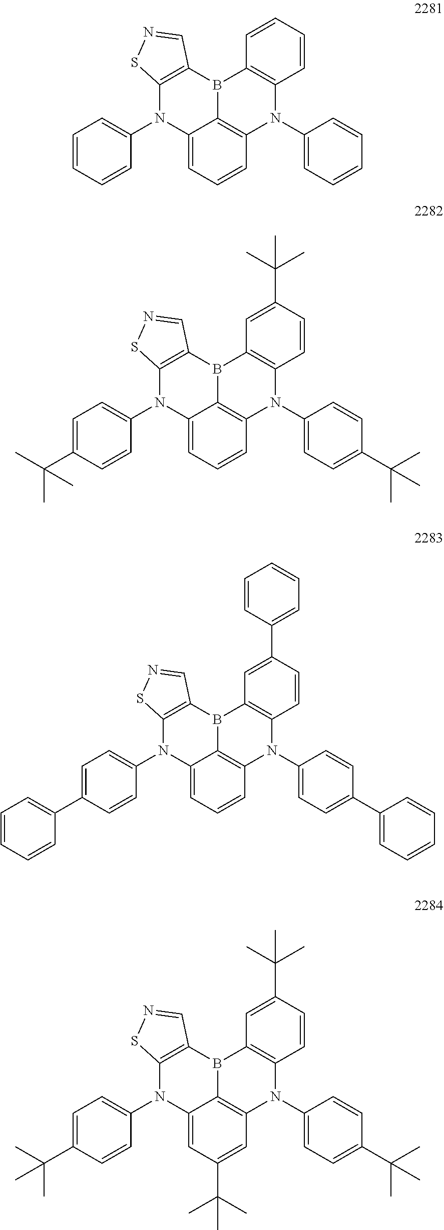

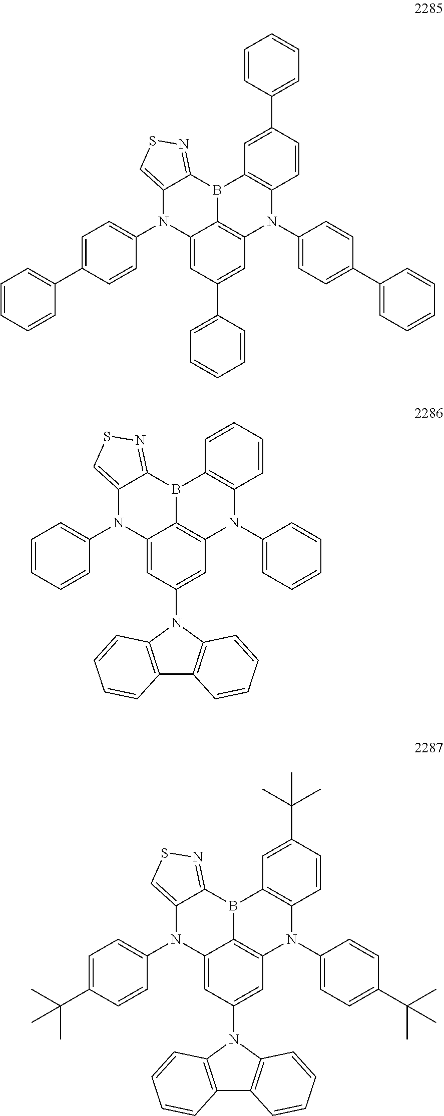 [SMILES: c1ccc(-c2ccc(N3c4ccc(-c5ccccc5)cc4B4c5nscc5N(c5ccc(-c6ccccc6)cc5)c5cc(-c6ccccc6)cc3c54)cc2)cc1 ; c1ccc(N2c3ccccc3B3c4nscc4N(c4ccccc4)c4cc(-n5c6ccccc6c6ccccc65)cc2c43)cc1 ; CC(C)(C)c1ccc(N2c3ccc(C(C)(C)C)cc3B3c4nscc4N(c4ccc(C(C)(C)C)cc4)c4cc(-n5c6ccccc6c6ccccc65)cc2c43)cc1]

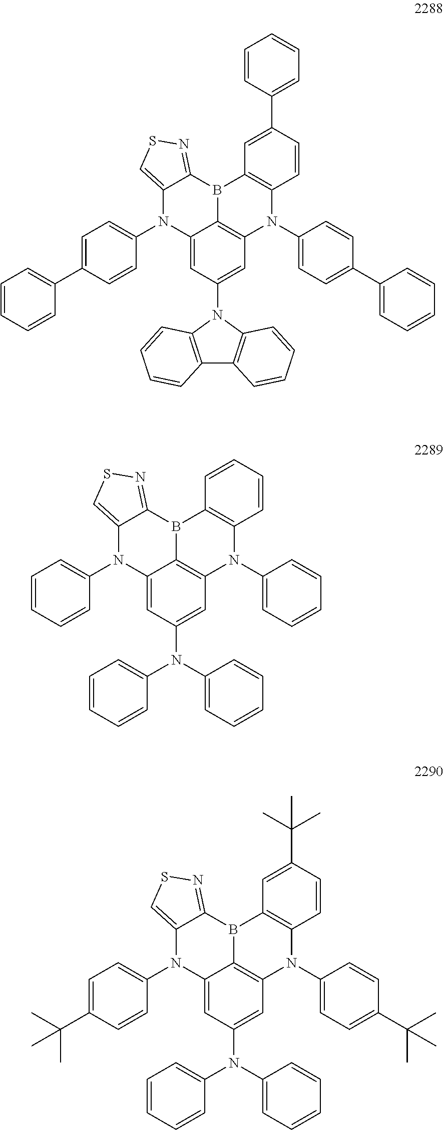 [SMILES: c1ccc(-c2ccc(N3c4ccc(-c5ccccc5)cc4B4c5nscc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2csnc2B4c2ccccc2N3c2ccccc2)cc1 ; CC(C)(C)c1ccc(N2c3ccc(C(C)(C)C)cc3B3c4nscc4N(c4ccc(C(C)(C)C)cc4)c4cc(N(c5ccccc5)c5ccccc5)cc2c43)cc1]

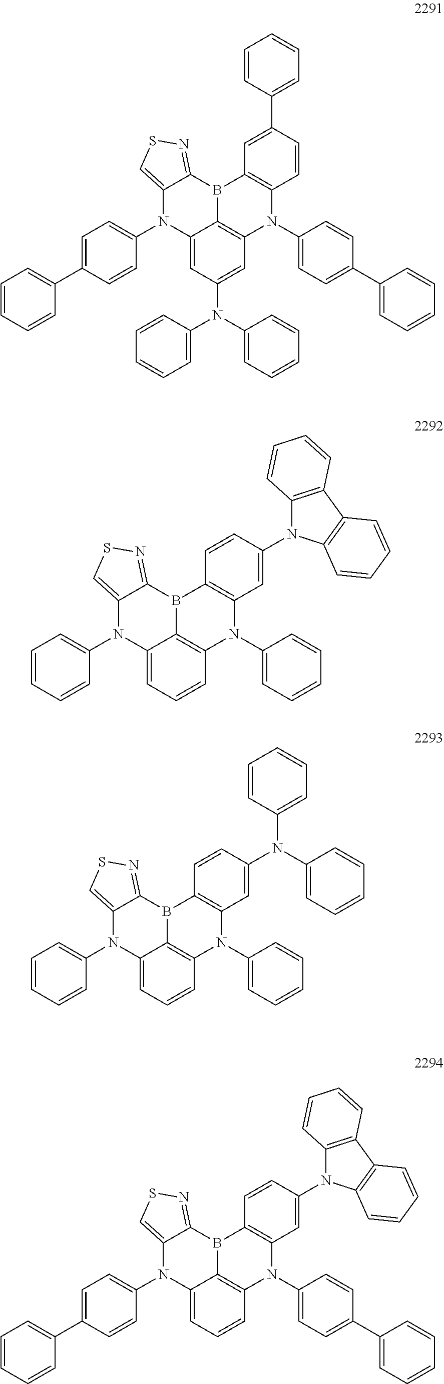 [SMILES: c1ccc(-c2ccc(N3c4ccc(-c5ccccc5)cc4B4c5nscc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nscc4N(c4ccccc4)c4cccc2c43)cc1 ; c1ccc(N(c2ccccc2)c2ccc3c(c2)N(c2ccccc2)c2cccc4c2B3c2nscc2N4c2ccccc2)cc1 ; c1ccc(-c2ccc(N3c4cc(-n5c6ccccc6c6ccccc65)ccc4B4c5nscc5N(c5ccc(-c6ccccc6)cc5)c5cccc3c54)cc2)cc1]

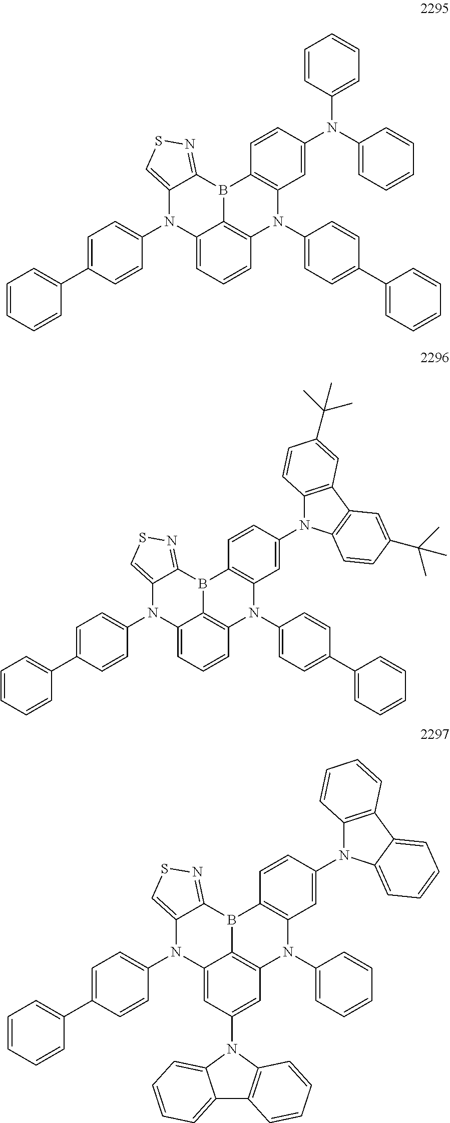 [SMILES: c1ccc(-c2ccc(N3c4cc(N(c5ccccc5)c5ccccc5)ccc4B4c5nscc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nscc1N3c1ccc(-c2ccccc2)cc1 ; c1ccc(-c2ccc(N3c4csnc4B4c5ccc(-n6c7ccccc7c7ccccc76)cc5N(c5ccccc5)c5cc(-n6c7ccccc7c7ccccc76)cc3c54)cc2)cc1]

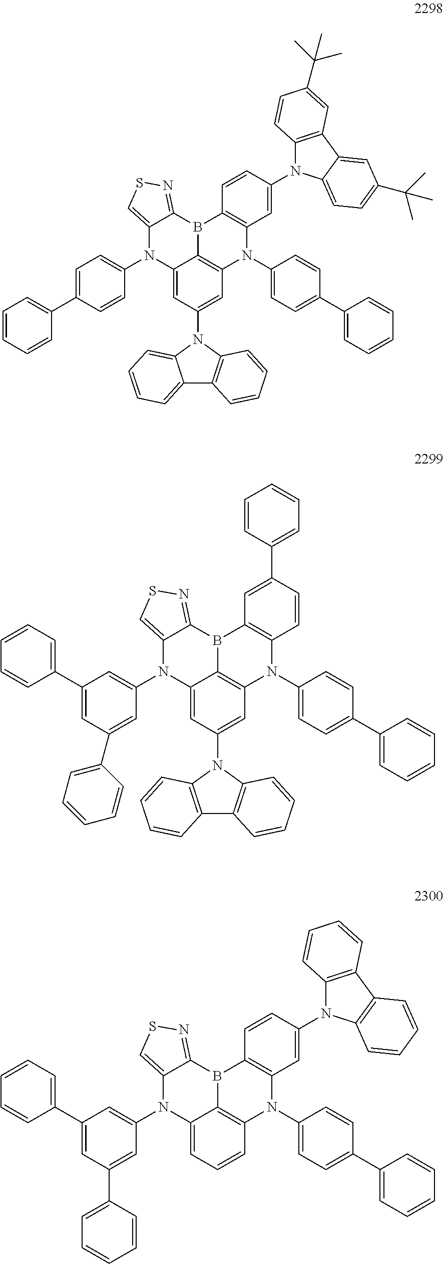 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nscc1N3c1ccc(-c2ccccc2)cc1 ; c1ccc(-c2ccc(N3c4ccc(-c5ccccc5)cc4B4c5nscc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nscc5N(c5cc(-c6ccccc6)cc(-c6ccccc6)c5)c5cccc3c54)cc2)cc1]

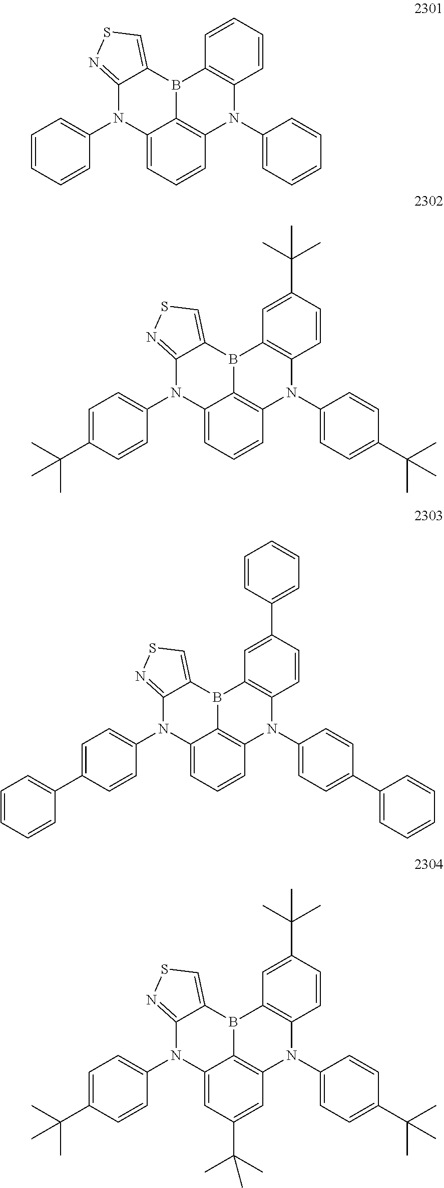

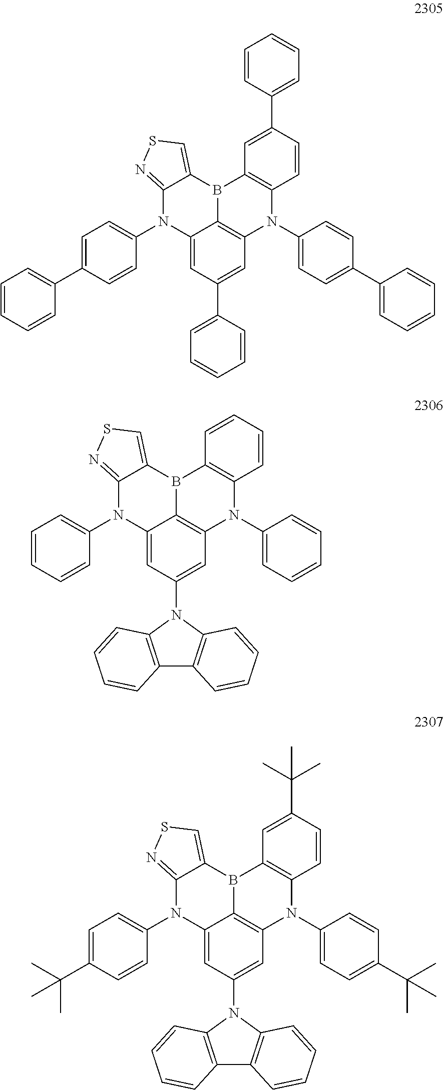

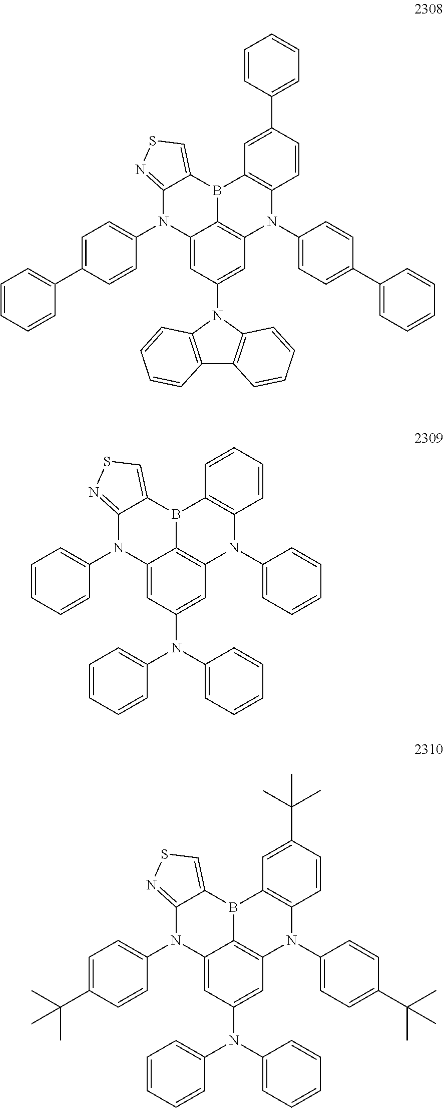 [SMILES: c1ccc(-c2ccc(N3c4ccc(-c5ccccc5)cc4B4c5csnc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2nscc2B4c2ccccc2N3c2ccccc2)cc1 ; CC(C)(C)c1ccc(N2c3ccc(C(C)(C)C)cc3B3c4csnc4N(c4ccc(C(C)(C)C)cc4)c4cc(N(c5ccccc5)c5ccccc5)cc2c43)cc1]

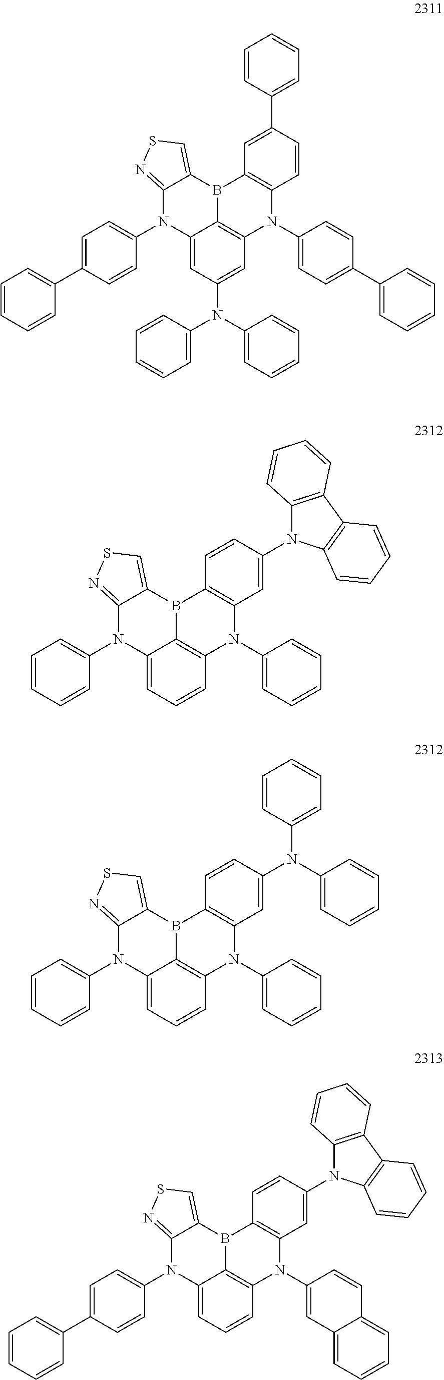 [SMILES: c1ccc(-c2ccc(N3c4ccc(-c5ccccc5)cc4B4c5csnc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4csnc4N(c4ccccc4)c4cccc2c43)cc1 ; c1ccc(N(c2ccccc2)c2ccc3c(c2)N(c2ccccc2)c2cccc4c2B3c2csnc2N4c2ccccc2)cc1 ; c1ccc(-c2ccc(N3c4cccc5c4B(c4ccc(-n6c7ccccc7c7ccccc76)cc4N5c4ccc5ccccc5c4)c4csnc43)cc2)cc1]

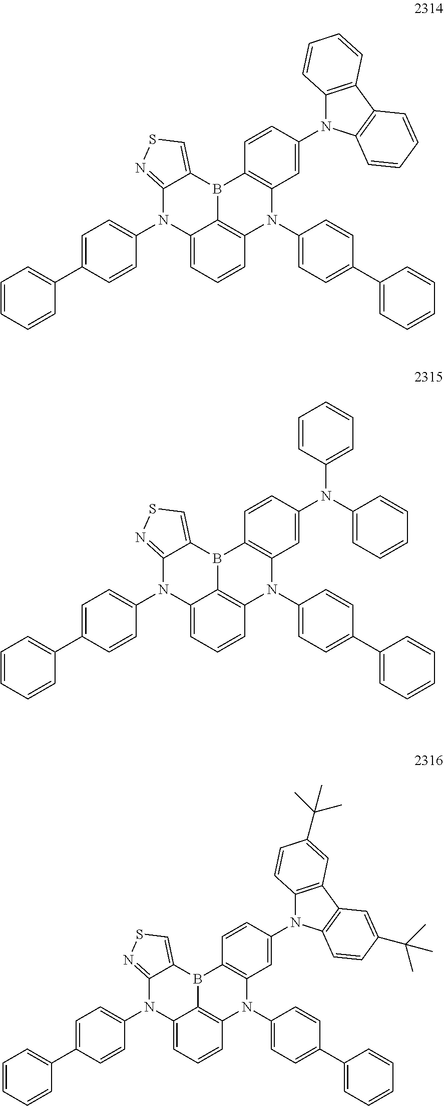 [SMILES: c1ccc(-c2ccc(N3c4cc(-n5c6ccccc6c6ccccc65)ccc4B4c5csnc5N(c5ccc(-c6ccccc6)cc5)c5cccc3c54)cc2)cc1 ; c1ccc(-c2ccc(N3c4cc(N(c5ccccc5)c5ccccc5)ccc4B4c5csnc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1csnc1N3c1ccc(-c2ccccc2)cc1]

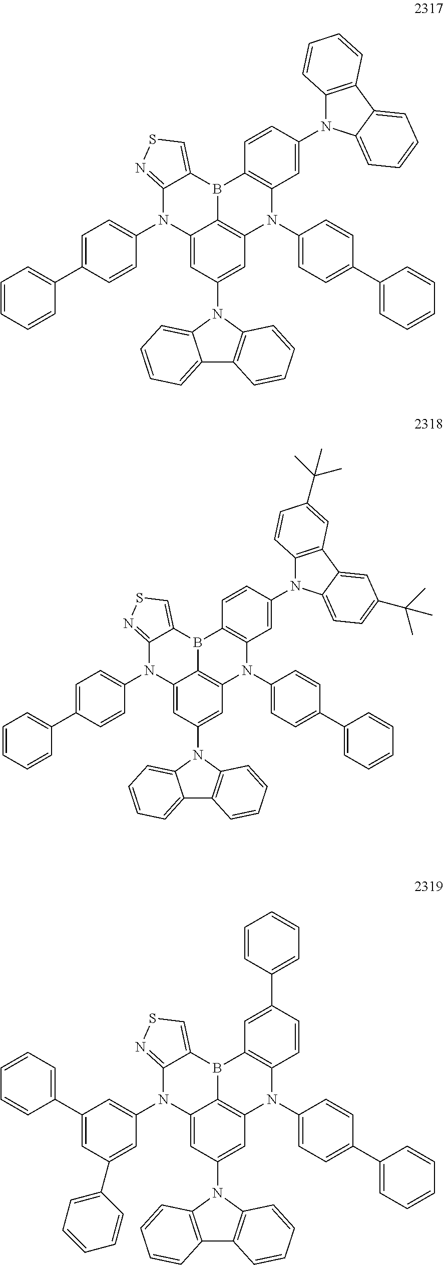 [SMILES: c1ccc(-c2ccc(N3c4cc(-n5c6ccccc6c6ccccc65)ccc4B4c5csnc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1csnc1N3c1ccc(-c2ccccc2)cc1 ; c1ccc(-c2ccc(N3c4ccc(-c5ccccc5)cc4B4c5csnc5N(c5cc(-c6ccccc6)cc(-c6ccccc6)c5)c5cc(-n6c7ccccc7c7ccccc76)cc3c54)cc2)cc1]

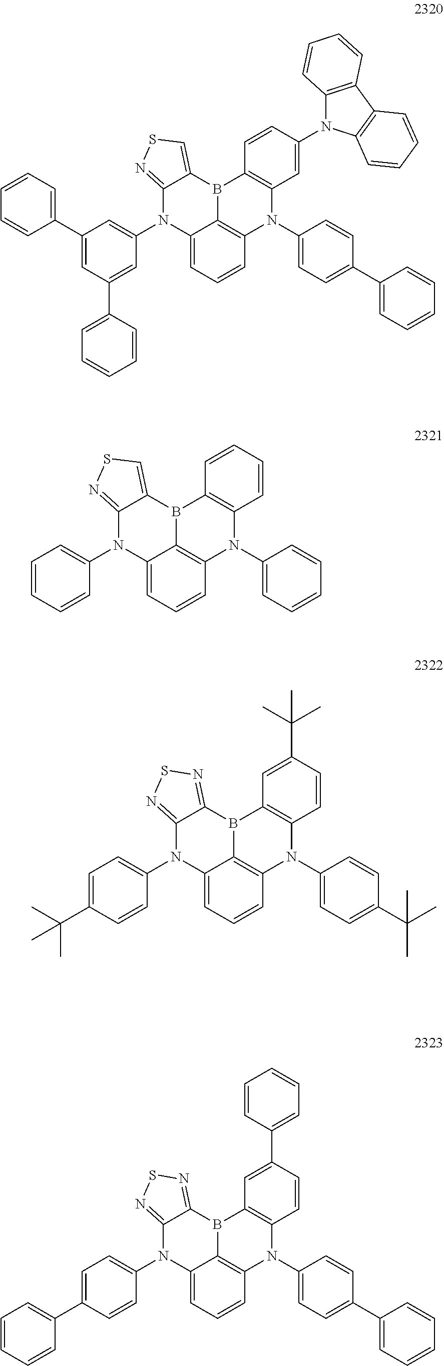 [SMILES: c1ccc(-c2ccc(N3c4cc(-n5c6ccccc6c6ccccc65)ccc4B4c5csnc5N(c5cc(-c6ccccc6)cc(-c6ccccc6)c5)c5cccc3c54)cc2)cc1 ; c1ccc(N2c3ccccc3B3c4csnc4N(c4ccccc4)c4cccc2c43)cc1 ; CC(C)(C)c1ccc(N2c3ccc(C(C)(C)C)cc3B3c4nsnc4N(c4ccc(C(C)(C)C)cc4)c4cccc2c43)cc1 ; c1ccc(-c2ccc(N3c4ccc(-c5ccccc5)cc4B4c5nsnc5N(c5ccc(-c6ccccc6)cc5)c5cccc3c54)cc2)cc1]

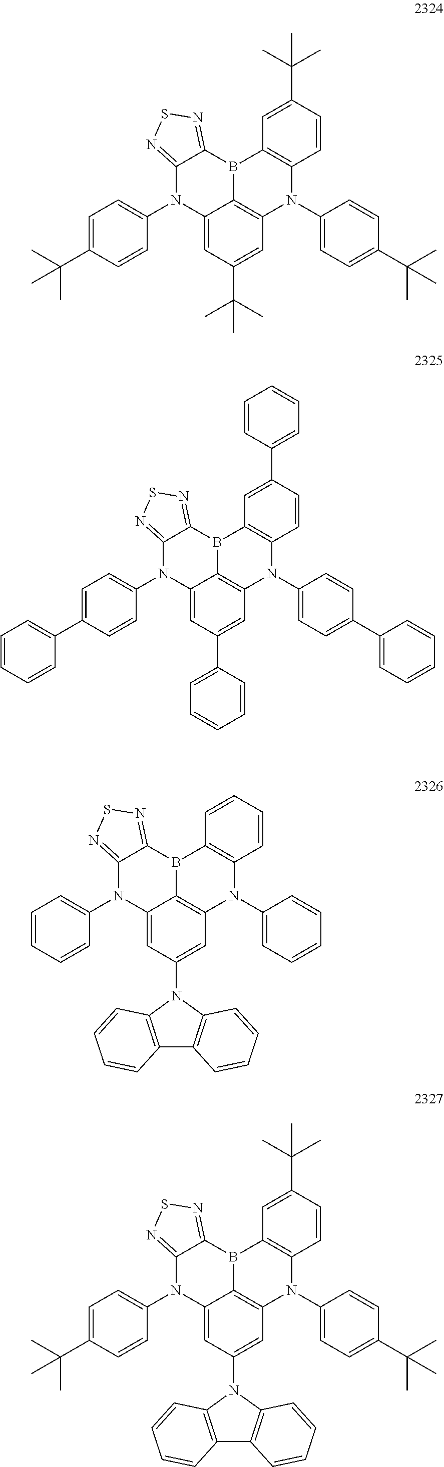

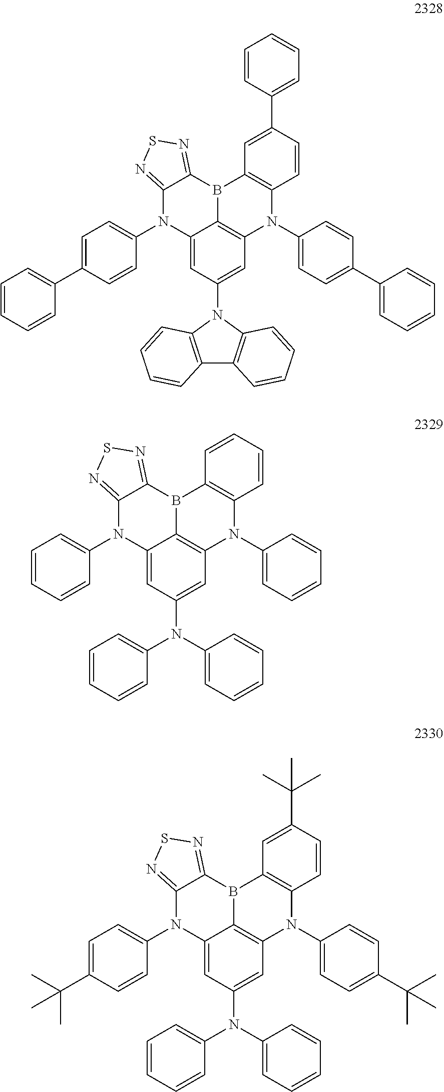 [SMILES: c1ccc(-c2ccc(N3c4ccc(-c5ccccc5)cc4B4c5nsnc5N(c5ccc(-c6ccccc6)cc5)c5cc(-n6c7ccccc7c7ccccc76)cc3c54)cc2)cc1 ; c1ccc(N(c2ccccc2)c2cc3c4c(c2)N(c2ccccc2)c2nsnc2B4c2ccccc2N3c2ccccc2)cc1 ; CC(C)(C)c1ccc(N2c3ccc(C(C)(C)C)cc3B3c4nsnc4N(c4ccc(C(C)(C)C)cc4)c4cc(N(c5ccccc5)c5ccccc5)cc2c43)cc1]

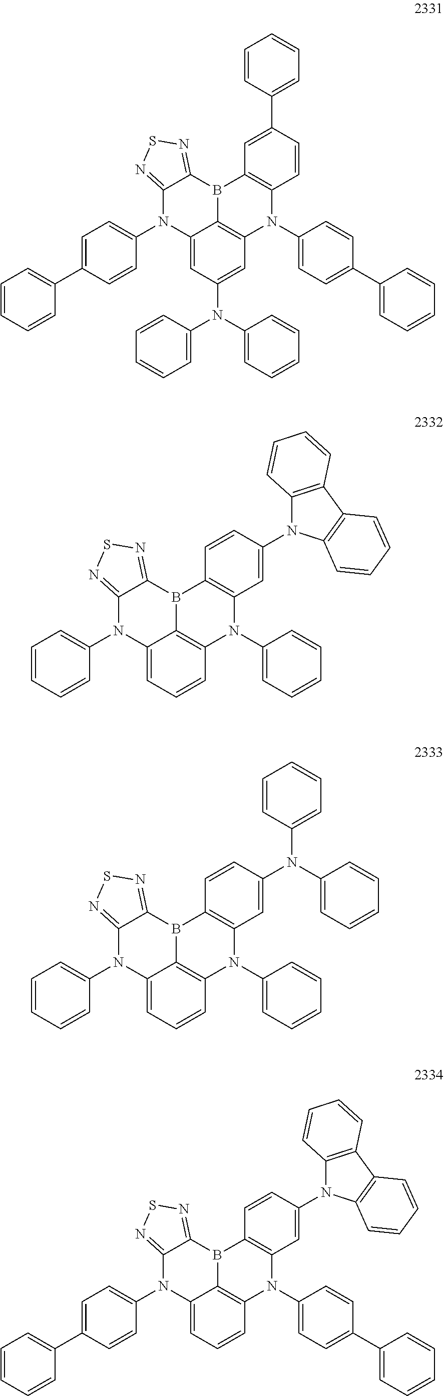 [SMILES: c1ccc(-c2ccc(N3c4ccc(-c5ccccc5)cc4B4c5nsnc5N(c5ccc(-c6ccccc6)cc5)c5cc(N(c6ccccc6)c6ccccc6)cc3c54)cc2)cc1 ; c1ccc(N2c3cc(-n4c5ccccc5c5ccccc54)ccc3B3c4nsnc4N(c4ccccc4)c4cccc2c43)cc1 ; c1ccc(N(c2ccccc2)c2ccc3c(c2)N(c2ccccc2)c2cccc4c2B3c2nsnc2N4c2ccccc2)cc1 ; c1ccc(-c2ccc(N3c4cc(-n5c6ccccc6c6ccccc65)ccc4B4c5nsnc5N(c5ccc(-c6ccccc6)cc5)c5cccc3c54)cc2)cc1]

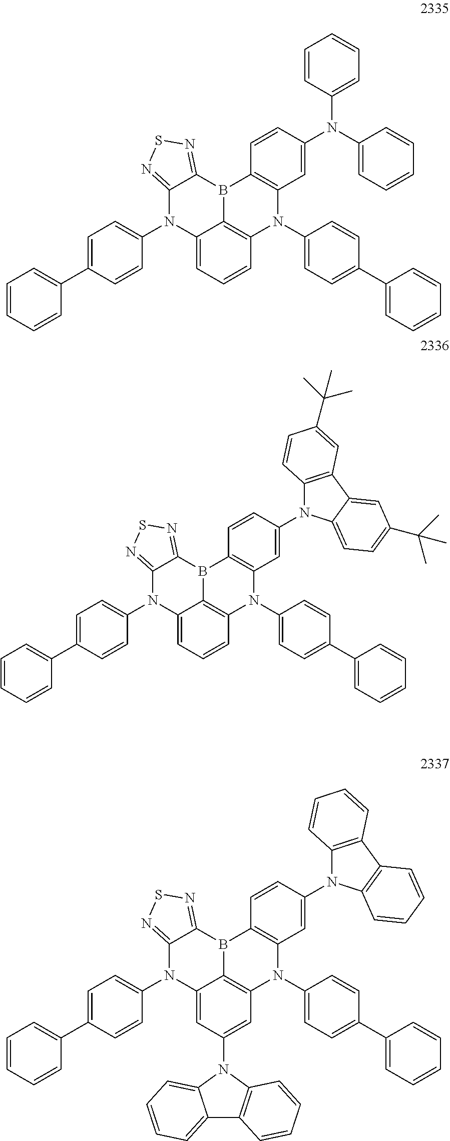 [SMILES: c1ccc(-c2ccc(N3c4cc(N(c5ccccc5)c5ccccc5)ccc4B4c5nsnc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1nsnc1N3c1ccc(-c2ccccc2)cc1 ; c1ccc(-c2ccc(N3c4cc(-n5c6ccccc6c6ccccc65)ccc4B4c5nsnc5N(c5ccc(-c6ccccc6)cc5)c5cc(-n6c7ccccc7c7ccccc76)cc3c54)cc2)cc1]

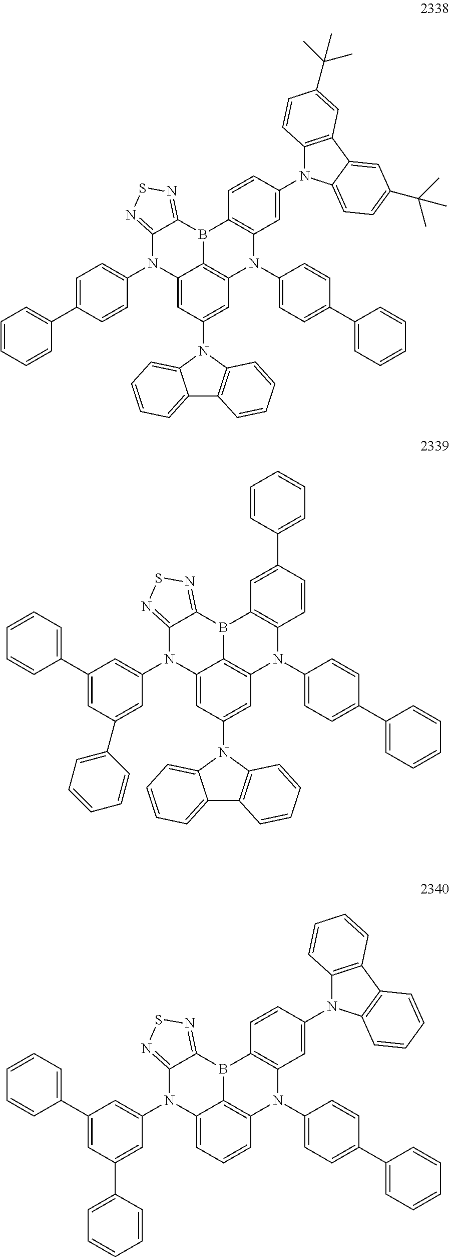 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1nsnc1N3c1ccc(-c2ccccc2)cc1 ; c1ccc(-c2ccc(N3c4ccc(-c5ccccc5)cc4B4c5nsnc5N(c5cc(-c6ccccc6)cc(-c6ccccc6)c5)c5cc(-n6c7ccccc7c7ccccc76)cc3c54)cc2)cc1 ; c1ccc(-c2ccc(N3c4cc(-n5c6ccccc6c6ccccc65)ccc4B4c5nsnc5N(c5cc(-c6ccccc6)cc(-c6ccccc6)c5)c5cccc3c54)cc2)cc1]

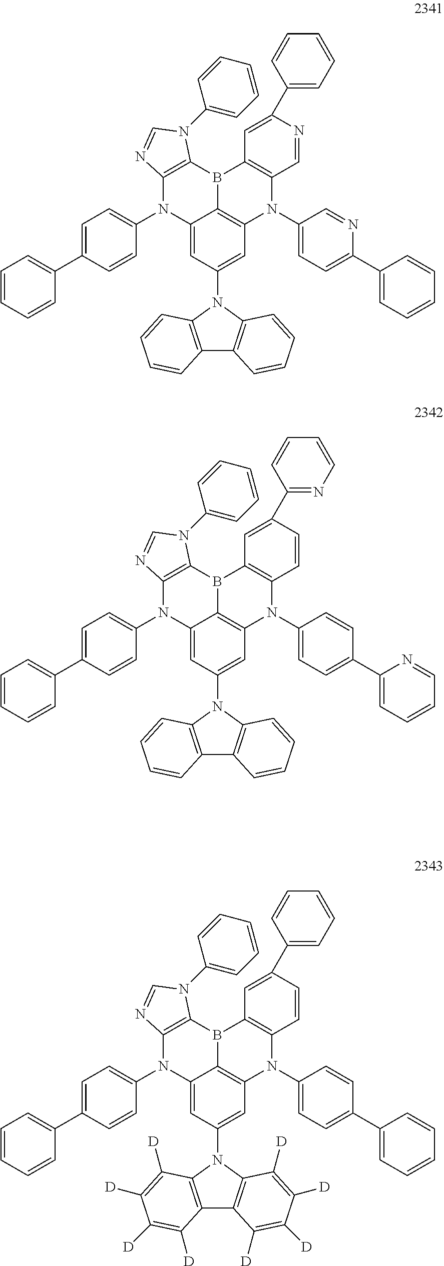 [SMILES: c1ccc(-c2ccc(N3c4cc(-n5c6ccccc6c6ccccc65)cc5c4B(c4cc(-c6ccccc6)ncc4N5c4ccc(-c5ccccc5)nc4)c4c3ncn4-c3ccccc3)cc2)cc1 ; c1ccc(-c2ccc(N3c4cc(-n5c6ccccc6c6ccccc65)cc5c4B(c4cc(-c6ccccn6)ccc4N5c4ccc(-c5ccccn5)cc4)c4c3ncn4-c3ccccc3)cc2)cc1 ; [2H]c1c([2H])c([2H])c2c(c1[2H])c1c([2H])c([2H])c([2H])c([2H])c1n2-c1cc2c3c(c1)N(c1ccc(-c4ccccc4)cc1)c1ncn(-c4ccccc4)c1B3c1cc(-c3ccccc3)ccc1N2c1ccc(-c2ccccc2)cc1]

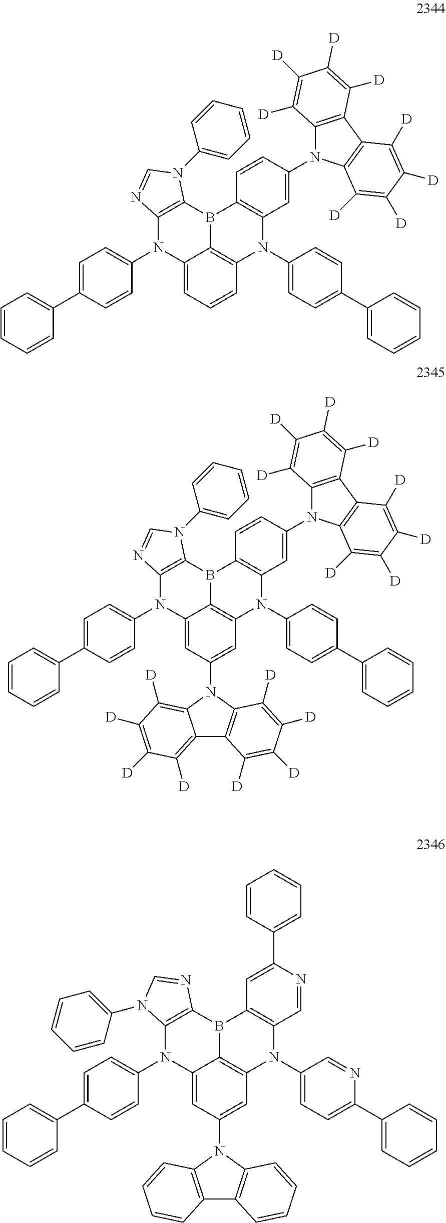 [SMILES: [2H]c1c([2H])c([2H])c2c(c1[2H])c1c([2H])c([2H])c([2H])c([2H])c1n2-c1ccc2c(c1)N(c1ccc(-c3ccccc3)cc1)c1cccc3c1B2c1c(ncn1-c1ccccc1)N3c1ccc(-c2ccccc2)cc1 ; [2H]c1c([2H])c([2H])c2c(c1[2H])c1c([2H])c([2H])c([2H])c([2H])c1n2-c1ccc2c(c1)N(c1ccc(-c3ccccc3)cc1)c1cc(-n3c4c([2H])c([2H])c([2H])c([2H])c4c4c([2H])c([2H])c([2H])c([2H])c43)cc3c1B2c1c(ncn1-c1ccccc1)N3c1ccc(-c2ccccc2)cc1 ; c1ccc(-c2ccc(N3c4cc(-n5c6ccccc6c6ccccc65)cc5c4B(c4cc(-c6ccccc6)ncc4N5c4ccc(-c5ccccc5)nc4)c4ncn(-c5ccccc5)c43)cc2)cc1]

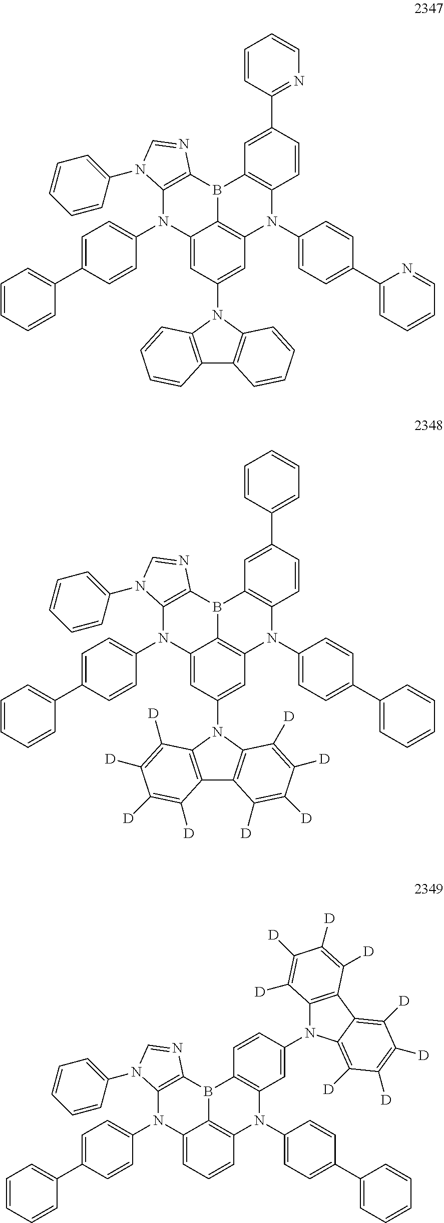 [SMILES: c1ccc(-c2ccc(N3c4cc(-n5c6ccccc6c6ccccc65)cc5c4B(c4cc(-c6ccccn6)ccc4N5c4ccc(-c5ccccn5)cc4)c4ncn(-c5ccccc5)c43)cc2)cc1 ; [2H]c1c([2H])c([2H])c2c(c1[2H])c1c([2H])c([2H])c([2H])c([2H])c1n2-c1cc2c3c(c1)N(c1ccc(-c4ccccc4)cc1)c1c(ncn1-c1ccccc1)B3c1cc(-c3ccccc3)ccc1N2c1ccc(-c2ccccc2)cc1 ; [2H]c1c([2H])c([2H])c2c(c1[2H])c1c([2H])c([2H])c([2H])c([2H])c1n2-c1ccc2c(c1)N(c1ccc(-c3ccccc3)cc1)c1cccc3c1B2c1ncn(-c2ccccc2)c1N3c1ccc(-c2ccccc2)cc1]

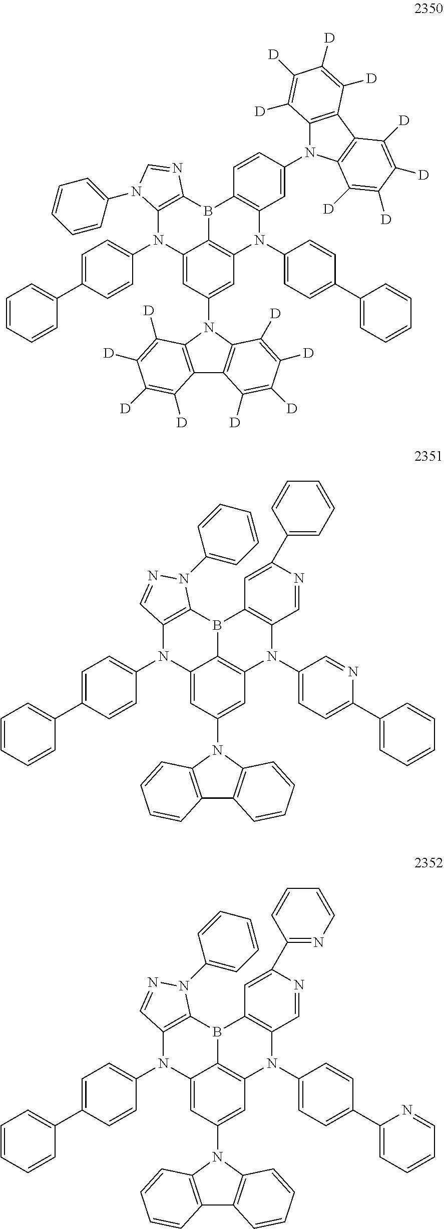 [SMILES: [2H]c1c([2H])c([2H])c2c(c1[2H])c1c([2H])c([2H])c([2H])c([2H])c1n2-c1ccc2c(c1)N(c1ccc(-c3ccccc3)cc1)c1cc(-n3c4c([2H])c([2H])c([2H])c([2H])c4c4c([2H])c([2H])c([2H])c([2H])c43)cc3c1B2c1ncn(-c2ccccc2)c1N3c1ccc(-c2ccccc2)cc1 ; c1ccc(-c2ccc(N3c4cc(-n5c6ccccc6c6ccccc65)cc5c4B(c4cc(-c6ccccc6)ncc4N5c4ccc(-c5ccccc5)nc4)c4c3cnn4-c3ccccc3)cc2)cc1 ; c1ccc(-c2ccc(N3c4cc(-n5c6ccccc6c6ccccc65)cc5c4B(c4cc(-c6ccccn6)ncc4N5c4ccc(-c5ccccn5)cc4)c4c3cnn4-c3ccccc3)cc2)cc1]

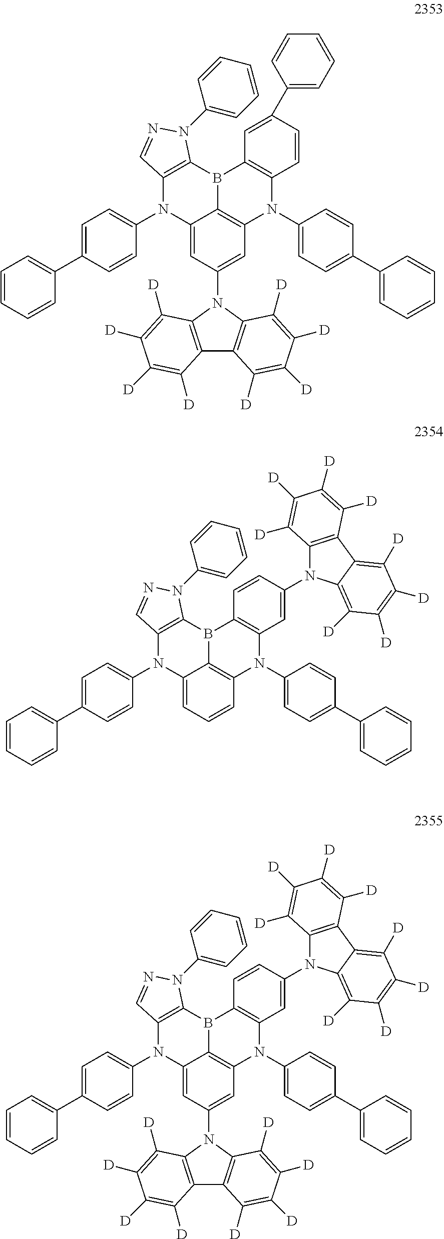

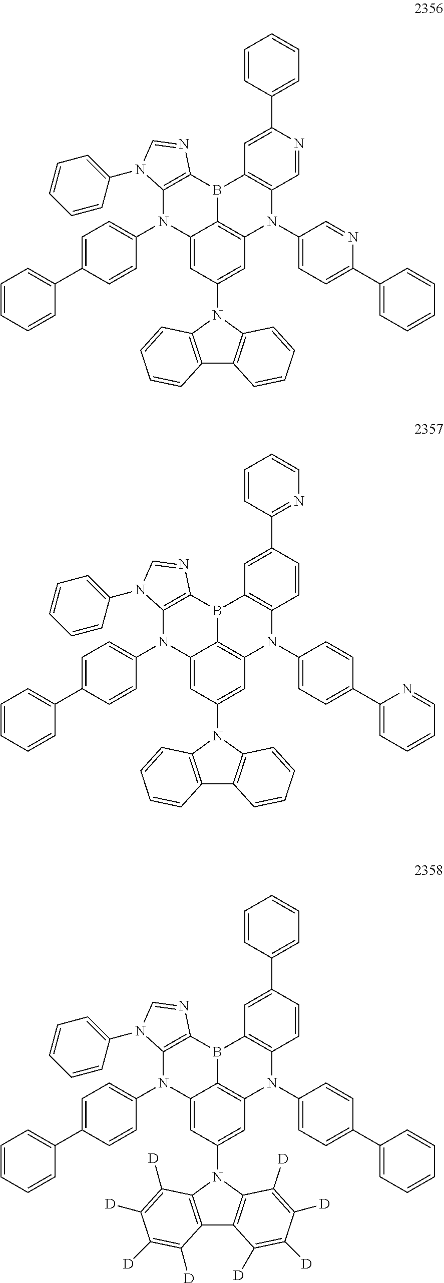 [SMILES: c1ccc(-c2ccc(N3c4cc(-n5c6ccccc6c6ccccc65)cc5c4B(c4cc(-c6ccccc6)ncc4N5c4ccc(-c5ccccc5)nc4)c4ncn(-c5ccccc5)c43)cc2)cc1 ; c1ccc(-c2ccc(N3c4cc(-n5c6ccccc6c6ccccc65)cc5c4B(c4cc(-c6ccccn6)ccc4N5c4ccc(-c5ccccn5)cc4)c4ncn(-c5ccccc5)c43)cc2)cc1 ; [2H]c1c([2H])c([2H])c2c(c1[2H])c1c([2H])c([2H])c([2H])c([2H])c1n2-c1cc2c3c(c1)N(c1ccc(-c4ccccc4)cc1)c1c(ncn1-c1ccccc1)B3c1cc(-c3ccccc3)ccc1N2c1ccc(-c2ccccc2)cc1]

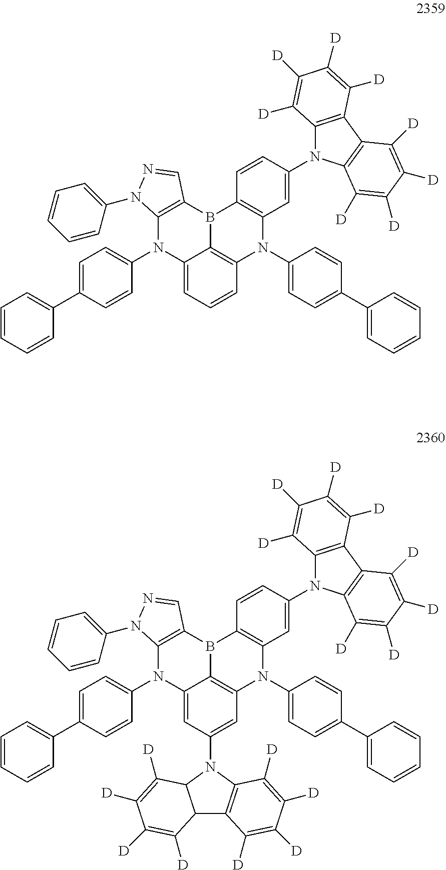 [SMILES: [2H]c1c([2H])c([2H])c2c(c1[2H])c1c([2H])c([2H])c([2H])c([2H])c1n2-c1ccc2c(c1)N(c1ccc(-c3ccccc3)cc1)c1cccc3c1B2c1cnn(-c2ccccc2)c1N3c1ccc(-c2ccccc2)cc1 ; [2H]C1=C([2H])C2c3c([2H])c([2H])c([2H])c([2H])c3N(c3cc4c5c(c3)N(c3ccc(-c6ccccc6)cc3)c3c(cnn3-c3ccccc3)B5c3ccc(-n5c6c([2H])c([2H])c([2H])c([2H])c6c6c([2H])c([2H])c([2H])c([2H])c65)cc3N4c3ccc(-c4ccccc4)cc3)C2C([2H])=C1[2H]]

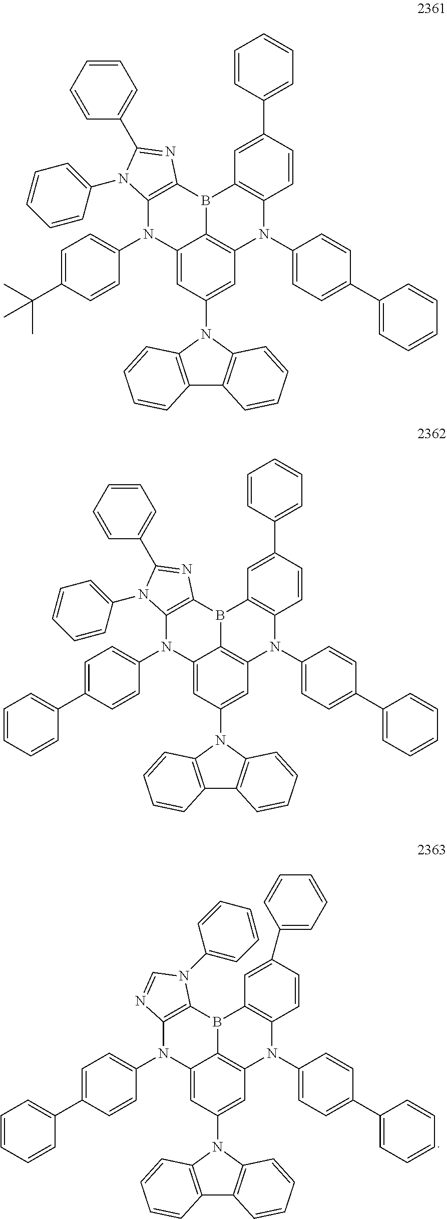 [SMILES: CC(C)(C)c1ccc(N2c3cc(-n4c5ccccc5c5ccccc54)cc4c3B(c3cc(-c5ccccc5)ccc3N4c3ccc(-c4ccccc4)cc3)c3nc(-c4ccccc4)n(-c4ccccc4)c32)cc1 ; c1ccc(-c2ccc(N3c4ccc(-c5ccccc5)cc4B4c5nc(-c6ccccc6)n(-c6ccccc6)c5N(c5ccc(-c6ccccc6)cc5)c5cc(-n6c7ccccc7c7ccccc76)cc3c54)cc2)cc1 ; c1ccc(-c2ccc(N3c4ccc(-c5ccccc5)cc4B4c5c3cc(-n3c6ccccc6c6ccccc63)cc5N(c3ccc(-c5ccccc5)cc3)c3ncn(-c5ccccc5)c34)cc2)cc1]

13. An organic light-emitting device comprising:
a first electrode;
a second electrode; and
an organic layer located between the first electrode and the second electrode and including an emission layer, wherein
the organic layer comprises at least one of the condensed cyclic compound.

14. The organic light-emitting device of claim 13, wherein
the at least one of the condensed cyclic compound is included in the emission layer.

15. The organic light-emitting device of claim 13, wherein
the emission layer comprises a host and a dopant, and
the dopant comprises at least one of the condensed cyclic compound.

16. The organic light-emitting device of claim 15, wherein
delayed fluorescence is emitted from the emission layer.

17. The organic light-emitting device of claim 15, wherein
blue light is emitted from the emission layer.

18. The organic light-emitting device of claim 13, wherein
the emission layer comprises a host, an emitter, and a sensitizer, wherein the host, the emitter, and the sensitizer are different from each other, and the at least one condensed cyclic compound is included in the emitter.

19. The organic light-emitting device of claim 18, wherein
the sensitizer and the condensed cyclic compound further satisfy Condition 5:
0 μs<T_{decay}(HC)<5 μs Condition 5
wherein, in Condition 5,
T_{decay}(HC) indicates a decay time of the condensed cyclic compound.

20. An electronic apparatus comprising the organic light-emitting device of claim 13.
